(19) United States
(12) Patent Application Publication
(10) Pub. No.: US 2021/0254056 A1
(43) Pub. Date: Aug. 19, 2021

(54) IDENTIFICATION AND TARGETED MODULATION OF GENE SIGNALING NETWORKS

(71) Applicant: CAMP4 THERAPEUTICS CORPORATION, Cambridge, MA (US)

(72) Inventors: Yuting LIU, Brookline, MA (US); Alla SIGOVA, Newton, MA (US); Charles W. O'DONNELL, Winchester, MA (US); Cynthia SMITH, Boston, MA (US); Gavin WHISSELL, North Reading, MA (US); Brett CHEVALIER, Melrose, MA (US); Jennifer F. BRYAN, Groton, MA (US); Donna T. WARD, Groton, MA (US)

(21) Appl. No.: 16/610,899

(22) PCT Filed: May 4, 2018
(86) PCT No.: PCT/US2018/031056

§ 371 Date: Nov. 4, 2019

Related U.S. Application Data
(60) Provisional application No. 62/501,795, filed on May 5, 2017.

(51) Int. Cl. C12N 15/113; G16B 20/30; G16B 20/50; G16B 25/10

(52) U.S. Cl. CPC C12N 15/113; G16B 20/30; G16B 20/50; C12N 2310/11; C12N 2310/14; C12N 2310/20; C12N 2320/12; G16B 25/10

(57) ABSTRACT

The present invention provides methods and compositions for the evaluation, alteration and/or optimization of gene signaling. Methods and systems are also provided which exploit the information generated in the identification of new targets and non-canonical signaling pathways.

### CROSS REFERENCE TO RELATED APPLICATIONS

The application claims priority to U.S. provisional application 62/501,795, filed May 5, 2017, entitled IDENTIFICATION AND TARGETED MODULATION OF GENE SIGNALING NETWORKS, the contents of which are hereby incorporated by reference herein in its entirety.

### SEQUENCE LISTING

The instant application contains a “lengthy” Sequence Listing which has been submitted via CD-R in lieu of a printed paper copy, and is hereby incorporated by reference in its entirety. Said CD-R, recorded on Apr. 5, 2018, are labeled “CRF,” “Copy 1−SEQUENCE LISTING PART,” and “Copy 2—SEQUENCE LISTING PART” and “Copy 3—SEQUENCE LISTING PART”, respectively, and each contains only one identical 233,328,640 bytes file (2093_1300 PCT_SL.txt).

### FIELD OF THE INVENTION

The invention relates to the identification, mapping, and optimization of gene expression mechanisms, such as genomic signaling centers (GSCs), in biological systems. Further, the invention relates to methods and compositions for identifying the location of occupancy-dependent signaling centers (ODSCs), and methods and compositions for altering the binding profile of the ODSC. These methods are useful in the identification of heretofore unidentified targets for disease.

### BACKGROUND OF THE INVENTION

It is now understood that a majority of human diseases have a genetic component. Common diseases such as diabetes, cancer, some types of heart diseases, and arthritis, which typically arise later in life, may run in families and often have a genetic component or driver.

Inherited genetic diseases can be fatal or result in conditions that require significant medical intervention. Amongst inherited genetic diseases, rare inherited genetic diseases represent a greater medical challenge. There is presently limited ability to approach therapies for inherited diseases, especially rare inherited diseases.

A variety of causative mutations or variants associated with inherited genetic diseases have been mapped to regions in the genome known as genomic signaling centers, or areas of active remodeling or occupancy by factors and biomolecules thought to be responsible for, or involved in, gene expression from nearby genes. Sets of genes associated in some way with genomic signaling centers and under similar control mechanisms have been identified in the literature and have been termed “insulated neighborhoods” (INs). The integrity of a single or multiple INs is important for proper expression of local genes. Disruption of GSCs or INs is thought to be involved in disease progression, pathogenesis and/or etiology.

Studies to map, with high fidelity, the relationships among chromatin status, structure and organization, GSCs, INs, gene expression, cell identity and disease are critical to understanding not only how therapeutic compounds affect biological systems but to help predict therapeutic outcomes of treatment modalities a priori and to aid in patient selection and/or stratification for such treatments. To date, such efforts have been only somewhat successful, owing, to some degree, to the current nature of describing, and hence studying, “canonical” gene signaling pathways. Canonical pathways described in scientific literature, e.g., the MAP Kinase pathway, NF-kappaB pathway, insulin signaling pathway, or integrin signaling pathway, are not self-contained pathways, per se. A subset of proteins and/or nucleic acids in any one pathway may also participate in the signaling of another pathway. In essence, the current paradigm of describing gene signaling is fraught with inherent uncertainties as selected pathway components exhibit a high degree of promiscuity. What is needed is an alternative approach to the characterization of signaling in which the actual signaling of any gene in response to any stimulus or perturbation may be described wholly and independent of canonical cascade labels. Such a system, while being more complex, would allow more efficient and informative hypothesis testing and therefore better prediction, stratification and/or mapping of cellular system response outcomes to various perturbations or stimuli. The present invention contemplates such an alternative approach as well as applications of such an approach in the fields of research, development and therapeutic treatment, diagnosis, prognosis and stratification.

Various embodiments of the invention provide for perturbation of cell signaling by disrupting the composition of molecules at GSCs, which may form an ODSC, by contacting the GSC with a stimulus, e.g., biomolecules such as for example small molecules, antibodies, or cell-penetrating polypeptides. Stimuli may also include alterations in the environment of a GSN such as a change in temperature, pH, a chemical change, or in some cases, no externally applied stimulus at all (e.g., null).

Control of cell signaling pathways may also be used to treat hundreds of inherited diseases caused by mutations in regulatory, non-coding regions by controlling gene expression. Even a seemingly slight change in gene expression has been shown to have a significant impact on diseases. Therefore, the applications of the invention extend beyond the boundaries of canonical cell signaling in that an alternative approach to the definition and description of “pathways” is provided, along with methods of studying the perturbation of such systems and the exploitation of the outcomes of such perturbations.

Perturbation of a genomic system using at least one of the stimuli described herein may result in a response such as the location of a GSC changing its relative location to an insulated neighborhood gene or the binding profile of the GSC forming an ODSC. Changes to GSCs may therefore be useful in the modulation of gene expression, mapping other GSCs in support of the creation of a map of gene signaling networks, personalized medicine, and as a drug discovery platform.

### SUMMARY OF THE INVENTION

The invention provides, among other things, a method of altering signaling of a primary neighborhood gene encoded within an insulated neighborhood. Such methods include, but are not limited to disrupting a primary upstream or primary downstream boundary of the insulated neighborhood; altering one or more regulatory sequence regions (RSRs) or portions thereof of the encoded primary neighborhood gene; duplicating one or more RSRs or portions thereof of the encoded primary neighborhood gene; inhibiting or reducing the expression and/or activity of one or more signaling molecules associated with the RSR of the primary neighborhood gene; activating or increasing the expression and/or activity of one or more signaling molecules associated with the RSR of the primary neighborhood gene; and/or altering one or more of the upstream or downstream neighborhood genes or its RSR of the insulated neighborhood. Such methods may further comprise contacting a genomic system that includes the insulated neighborhood with a stimulus. Such stimuli include any of those taught herein.

In some embodiments, the insulated neighborhood is a minimal insulated neighborhood.

In some embodiments, the method further includes contacting a genomic system that includes the insulated neighborhood with a stimulus. The stimulus may be a small molecule, a biologic, an antibody, an environmental condition, and a combination thereof. In some embodiments, the stimulus is selected from those described herein.

In some embodiments, the invention includes an isolated cell having at least one insulated neighborhood altered in any manner.

In some embodiments, the present invention comprises methods of altering the penetrance of a gene comprising altering the structure of one or more insulated neighborhoods that encompass the gene.

In some embodiments, the present invention comprises a method of predicting one or more treatment liabilities of a therapeutic agent comprising determining the signaling signature of one or more primary neighborhood genes which are differentially expressed upon treatment with the therapeutic agent compared to an untreated control.

In some embodiments, the present invention comprises altering the signaling signature of one or more primary neighborhood genes which are differentially expressed upon treatment with a therapeutic agent compared to an untreated control. In some embodiments, the signaling signature is altered by a method selected from the group consisting of increasing the level of a signaling molecule, decreasing the level of a signaling molecule, editing one or more RSRs, altering an IN boundary, affecting a downstream target, and mutating a genomic signaling center. In some embodiments, the signaling molecules to be increased or decreased comprise one or more transcription factors selected from those listed in Table 22.

In some embodiments, the present invention comprises a method of reducing or eliminating one or more treatment liabilities of a therapeutic agent comprising altering the penetrance of a primary neighborhood gene or its RSRs. Such treatment liability may be selected from toxicity, short half-life, and lack of efficacy.

In some embodiments, the present invention comprises a method of altering expression of gene selected from any of those listed in Tables 1-9 in a liver cell comprising contacting said liver cell with a compound selected from the group consisting of any of those taught herein.

In some embodiments, the present invention comprises a method of stratifying or selecting patients for treatment with a compound selected from the group consisting of any of those taught herein.

In some embodiments, the present invention comprises a method of screening cell for response to a stimulus by measuring differential gene expression between a group of the cells contacted with the stimulus and a group of the cells not contacted with the stimulus, wherein the stimulus comprises any of those selected from the group consisting of any of any compound taught herein.

In some embodiments, the present invention comprises a method of altering the gene expression attendant to an insulated neighborhood comprising altering a genomic signaling center, the method comprising using a CRISPR/Cas9 system to change the genomic signaling center.

In some embodiments, the present invention comprises a method of creating new genomic signaling centers in a genome, the method comprising altering a CTCF site to disrupt an enhancer-promoter interaction of a first insulated neighborhood, wherein the enhancer is available for interaction with a different promoter to form a new genomic signaling center. In some embodiments, the CTCF site is altered using a CRISPR/Cas9 enzyme.

In some embodiments, the present invention comprises a method of modulating gene expression in a cell, the method including contacting the cell with a perturbation stimulus targeting at least one occupancy-dependent signaling center, wherein the occupancy-dependent signaling center comprises a region of the genome bound by at least 2 signaling proteins and comprises i) a H3K27 chemical modification, or ii) independently at least one of a bromodomain-containing protein (Brd), a transcriptional coactivator, and at least two master transcription factors bound to the region, thereby modulating gene expression. In some embodiments, the bromodomain-containing protein (Brd) is selected from the group consisting of Brd2, Brd3, and Brd4. In some embodiments, the H3K27 chemical modification is acetylation or methylation. In some embodiments, the transcriptional coactivator is p300. In some embodiments, the occupancy-dependent signaling center comprises a nucleic acid sequence selected from SEQ ID NOs: 32,627-71,281. In some embodiments, the perturbation stimulus is at least one selected from the group consisting of a CRISPR/Cas system, a zinc finger nuclease, a transcription activator-like effector nuclease (TALEN), and a hybridizing oligonucleotide. In some embodiments, the perturbation stimulus binds the occupancy-dependent signaling center. In some embodiments, the perturbation stimulus is antisense to the nucleic acid sequence of the occupancy-dependent signaling center.

In some embodiments, the method further includes mutating the nucleic acid sequence of the occupancy-dependent signaling center. In some embodiments, the contacting alters the occupancy of the occupancy-dependent signaling center. In some embodiments, the contacting alters genome architecture in the cell. In some embodiments, the contacting alters gene looping. In some embodiments, the perturbation stimulus is at least one stimulus described herein.

In some embodiments, the present invention comprises a method of altering the occupancy of a signaling center, the method including contacting the cell with a perturbation stimulus targeting at least one occupancy-dependent signaling center comprising a nucleic acid sequence selected from SEQ ID NOs: 32,627-71,281. In some embodiments, the perturbation stimulus is at least one selected from the group consisting of a CRISPR/Cas system, a zinc finger nuclease, a transcription activator-like effector nuclease (TALEN), and a hybridizing oligonucleotide. In some embodiments, the perturbation stimulus binds at least a portion of the occupancy-dependent signaling center. In some embodiments, the perturbation stimulus is antisense to the portion of the occupancy-dependent signaling center.

In some embodiments, the present invention comprises a composition comprising an oligonucleotide that binds to at least a portion of an occupancy-dependent signaling center comprising a nucleic acid sequence selected from SEQ ID NOs: 32,627-71,281. In some embodiments, the oligonucleotide is antisense to the nucleic acid sequence of the occupancy-dependent signaling center. In some embodiments, the present invention comprises a pharmaceutical composition comprising the composition of the oligonucleotide and a pharmaceutically acceptable excipient.

In some embodiments, the present invention comprises a method of treating a disease in a subject, the method comprising administering to the subject the pharmaceutical composition described herein, wherein the occupancy-dependent signaling center controls expression of at least one gene associated with the disease. In some embodiments, the gene is a protein-coding gene. H some embodiments, the gene is a non-protein-coding gene.

In some embodiments, the present invention comprises a method of perturbing a signaling pathway of a cell, the method including contacting the cell with a perturbation stimulus that alters the occupancy of an occupancy-dependent signaling center comprising a nucleic acid sequence selected from SEQ ID NOs: 32,627-71,281.

### BRIEF DESCRIPTION OF THE DRAWINGS

FIG. 1 illustrates the packaging of chromosomes in a nucleus, the localized topological domains into which chromosomes are organized, insulated neighborhoods in TADs and finally an example of an arrangement of a genomic signaling center(s) around a particular disease gene.

FIG. 2A and FIG. 2B illustrate a linear and 3D arrangement of the CTCF boundaries of an insulated neighborhood.

FIG. 3A and FIG. 3B illustrate tandem insulated neighborhoods and gene loops formed in such insulated neighborhoods.

FIG. 4 illustrates the concept of an insulated neighborhood contained within a larger insulated neighborhood and the signaling which may occur in each.

FIG. 5 illustrates the components of a genomic signaling center; including transcriptional factors, signaling proteins, and/or chromatin regulators.

FIG. 6A illustrates alternative signaling from insulated neighborhoods in different cell types, and FIG. 6B shows how a silent gene may become activated when the CTCF (cohesin) boundaries are disrupted.

FIG. 7 illustrates nested insulated neighborhoods (NI Ns) and four of the layers of studies performed to identify and map the GSNs the GSCs of the present invention.

FIG. 8 illustrates a “window” of the ChIP-seq array aligned with the identification of chromatin markers, transcription factor binding, signaling proteins for a section of a chromosome. The genes of interest are annotated along the length of the chromosome at the bottom against both the plus/minus or Watson/Crick strands. The coincident appearance of transcription factors between CTCF sites along the chromosome indicate potential genomic signaling centers for that neighborhood. The insulated neighborhood shown in the figure contains the SLC37A4 gene, implicated in glycogen storage disease 1b, and the RNA-seq results show upregulation of expression of the SLC37A4 gene by prednisone (MT-861), an immunosuppressant.

FIG. 9 is a scatterplot comparing disease associated gene (DAG) expression measured by RNA-seq and qRT-PCR.

FIG. 10A are ChIP-seq results for an insulated neighborhood containing the DNMBP gene. FIG. 10B are ChIP-seq results for an insulated neighborhood containing GADD45A gene. FIG. 10C are ChIP-seq results for an insulated neighborhood containing the POC1A gene and liver disease gene ALAS1.

FIG. 11A are ChIP-seq results for an insulated neighborhood including FKBP5, and RNA-seq results showing that prednisone (MT-861) also upregulates FKBP5, a gene implicated in immunosuppression. FIG. 11B are ChIP-seq results for an insulated neighborhood and RNA-seq results showing modulation of the COL1A1 gene implicated in liver fibrosis by BIO inhibitor (MT-209), a Wnt pathway agonist. FIG. 11C are ChIP-seq results for an insulated neighborhood and RNA-seq results showing modulation of the PCSK9 gene implicated in hypercholesterolemia by simvastatin (MT-51), a sterol agonist.

FIG. 12A are ChIP-seq results for an insulated containing HMOX1, MSM5, and RASD2 genes. FIG. 12B shows an insulated neighborhood containing the FOXA2 gene.

FIG. 13 shows ChIP-seq results for the insulated neighborhood containing PCSK9 and RNA-seq results showing modulation of expression of PCSK9 by prednisone (MT-861).

FIG. 14A is a graph of the size distribution of about 77 million paired end tags (PETs) identified by HiChIP. FIG. 14B is a heatmap of showing the presence of topologically-associated domains (TADs) and insulated neighborhoods.

FIG. 15 is an illustration comparing the number of cells throughout expansion and differentiation of DBA patient cells (dash line), the healthy patient cells (solid line), and the conditionally immortalized inducible DBA phenotype model disease model (dotted line).

FIG. 16A, FIG. 16B, and FIG. 16C are gene tracks of ChIP-seq results comparing the binding profiles during differentiation and expansion of CD34+ progenitor cells. FIG. 16A shows the insulated neighborhood including apoptosis-associated gene GADD45A. FIG. 16B shows the insulated neighborhood including p53 pathway-associated gene RPS19. FIG. 16C shows the insulated neighborhood including CDKN1A.

FIG. 17A, FIG. 17B, FIG. 170, FIG. 17D show the log 10 p-values for genomic annotation of the binding sites of p53 in hematopoietic stem cells (HSCs) and erythroid cells (log 10 p-values 4 were considered significant). FIG. 17A characterizes p53 binding sites in hematopoietic stem cell promoter regions. FIG. 17B characterizes p53 binding sites in erythroid cell promoter regions. FIG. 17C characterizes p53 binding sites in non-promoter regions of hematopoietic stem cells. FIG. 17D characterizes p53 binding sites in non-promoter regions of erythroid cells.

FIG. 18 illustrates one embodiment of the relational aspects of a gene signaling network (GSN) and associated mapping of the connectivities of such GSNs described by the invention.

FIG. 19A provides a graph of the log 2 number of signaling proteins per each signaling protein cluster. FIG. 19B provides a graph of the log 2 number of master transcription factors (TFs) per each master TF cluster from ChIP-seq results.

FIG. 20 is an exemplary illustration of the the HiChIP results showing genome interactions and architecture.

### DETAILED DESCRIPTION OF THE INVENTION

### I. Introduction

The present invention relates to the alteration, perturbation and ultimate regulated control of gene signaling networks (GSNs). Such gene signaling networks include genomic signaling centers (GSCs) found within insulated neighborhoods (I Ns) of the genomes of biological systems.

As used herein, a “gene signaling network” or “GSN” comprises the set of biomolecules associated with any or all of the signaling events from a particular gene, e.g., a gene-centric network. As there are over 20,000 protein-coding genes in the human genome, there are at least this many GSNs. And to the extent some genes are non-coding genes, the number increases greatly. Gene signaling networks differ from canonical signaling pathways which are mapped as standard protein cascades and feedback bops.

Traditionally, signaling pathways have been identified using standard biochemical techniques and, for the most part, are linear cascades with one protein product signaling the next protein product-driven event in the cascade. While these pathways may bifurcate or have feedback loops, the focus has been almost exclusively at the protein level.

GSNs of the present invention represent a different paradigm to defining biological signaling—taking into account protein-coding and nonprotein-coding signaling molecules/signaling proteins, genomic structure, chromosomal occupancy, chromosomal remodeling, the status of the biological system and the range of outcomes associated with the perturbation of any biological systems comprising such GSNs.

Genomic architecture, while not static, plays an important role in defining the framework of the GSNs of the present invention. Such architecture includes the concepts of chromosomal organization and modification, topologically associated domains (TADs), insulated neighborhoods (INs), genomic signaling centers (GSCs), signaling molecules/signalling proteins and their binding motifs or sites, and of course, the genes encoded within the genomic architecture. GSCs containing at least (i) 2 signaling proteins and comprises: (ii) a H3K27 chemical modification, or independently at least one of a bromodomain-containing protein (Brd), a transcriptional coactivator, and at least two master transcription factors bound to the region are referred to as an ODSC.

### Genomic Architecture

Cells control gene expression using thousands of elements that link cellular signaling to the architecture of the genome. Genomic system architecture includes regions of DNA, RNA transcripts, chromatin remodelers, and signaling molecules/signaling proteins.

### Chromosomes

Chromosomes are the largest subunit of genome architecture that contain most of the DNA in humans. Specific chromosome structures have been observed to play important roles in gene control, as described in Hnisz et al., Cell 167, Nov. 17, 2016, which is hereby incorporated by reference in its entirety. Non-coding regions including introns provide protein binding sites and other regulatory structures, while the exons encode for proteins including signaling molecules/signaling proteins, such as transcription factors, that interact with the non-coding regions to regulate gene expression. DNA sites within non-coding regions on the chromosome also interact with each other to form looped structures. These interactions form a chromosome scaffold that plays an important role in gene activation and repression. Interactions rarely occur among chromosomes and are usually within the same domain of a chromosome.

In situ hybridization techniques and microscopy have revealed that each interphase chromosome tends to occupy only small portion of the nucleus and does not spread throughout the whole organelle. See, Cremer and Cremer, Cold Spring Harbor Perspectives in Biology 2, a003889, 2010, which is hereby incorporated by reference in its entirety. This restricted surface occupancy area might reduce interactions between chromosomes.

Tables 1-9 lists genes found in the human genome. The methods described herein may be used to modulate the expression of any of these genes. As used herein, “expression” of a nucleic acid or gene sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Shown in Tables 1-9, in addition to the name and description of the gene (gene symbol) are the ENSEMBL Gene ID (ENSG), the gene found immediately upstream of the listed gene (Up ENSG) and the gene found immediately downstream of the listed gene (Down ENSG). It is noted that the unique identifiers for each ENSEMBL entry has been modified to remove the first five leading zeros (0) of the identifier after the ENSG label. Also given in the Tables are, in order of expression level, the tissues in which the transcripts are expressed. Table 1 provides the gene information for the genes found on Chromosome 1.

TABLE 1
Genes on Chromosome 1

| | | | Up | Down | Tissue |
|---|---|---|---|---|---|
| Gene Symbol | Gene Name | ENSG | ENSG | ENSG | Code |
| DHCR24 | 24-dehydrocholesterol reductase | 116133 | 162398 | 143001 | 2, 27, 43 |
| BPNT1 | 3′(2′),5′-bisphosphate nucleotidase 1 | 162813 | 136628 | 067704 | 13, 43, 45 |
| HMGCS2 | 3-hydroxy-3-methylglutaryl-CoA synthase 2 | 134240 | 092621 | 134193 | 19, 3, 38 |
| HMGCL | 3-hydroxymethyl-3-methylglutaryl-CoA lyase | 117305 | 117308 | 179163 | 3, 30, 17 |
| OXCT2 | 3-oxoacid CoA-transferase 2 | 198754 | 116985 | 043514 | 38, 28, 8 |
| HPDL | 4-hydroxyphenylpyruvate dioxygenase like | 186603 | 162415 | 132781 | 37, 9, 32 |
| HTR1D | 5-hydroxytryptamine receptor 1D | 179546 | 169641 | 125944 | 16, 14, 8 |
| HTR6 | 5-hydroxytryptamine receptor 6 | 158748 | 158747 | 162542 | 27, 23, |
| MTR | 5-methyltetrahydrofolate-homocysteine methyltransferase | 116984 | 077522 | 244020 | 2, 37, 11 |
| NT5C1A | 5′-nucleotidase, cytosolic IA | 116981 | 163909 | 116983 | |
| PFKFB2 | 6-phosphofructo-2-kinase/fructose-2,6-biphosphatase 2 | 123836 | 182795 | 180667 | |
| ABL2 | ABL proto-oncogene 2, non-receptor tyrosine kinase | 143322 | 186283 | 057252 | 34, 19, 25 |
| ASPM | abnormal spindle microtubule assembly | 066279 | 143278 | 177888 | 22, 13, 39 |
| AIM1L | absent in melanoma 1-like | 176092 | 169442 | 176083 | 24, 32, 2 |
| AIM2 | absent in melanoma 2 | 163568 | 163565 | 162706 | 22, 36, 30 |
| ASCL5 | achaete-scute family bHLH transcription factor 5 | 232237 | 081248 | 116857 | 16, |
| ACP6 | acid phosphatase 6, lysophosphatidic | 162836 | 116128 | 265107 | 9, 13, 42 |
| ANP32E | acidic nuclear phosphoprotein 32 family member E | 143401 | 023902 | 118298 | 34, 39, 11 |
| ACTL8 | actin like 8 | 117148 | 074964 | 117154 | 9, 38, 44 |
| ARPC5 | actin related protein 2/3 complex subunit 5 | 162704 | 116701 | 143344 | 43, 45, 13 |
| ACTRT2 | actin related protein T2 | 169717 | 215912 | 142611 | 38, 8, 12 |
| ACTA1 | actin, alpha 1, skeletal muscle | 143632 | 154429 | 069248 | 25, 18, 15 |
| ACTN2 | actinin alpha 2 | 077522 | 119285 | 116984 | 15, 18, 25 |
| ATF3 | activating transcription factor 3 | 162772 | 117691 | 162771 | 40, 26, 32 |
| ATF6 | activating transcription factor 6 | 118217 | 081721 | 162745 | 13, 45, 9 |
| ACBD3 | acyl-CoA binding domain containing 3 | 182827 | 163041 | 185155 | 29, 34, 1 |
| ACBD6 | acyl-CoA binding domain containing 6 | 230124 | 121454 | 143324 | |
| ACADM | acyl-CoA dehydrogenase, C-4 to C-12 straight chain | 117054 | 137968 | 137955 | 15, 1, 2 |
| ACOT11 | acyl-CoA thioesterase 11 | 162390 | 157216 | 162391 | 15, 1, 24 |
| ACOT7 | acyl-CoA thioesterase 7 | 097021 | 158292 | 069812 | 31, 26, 37 |
| ADAM15 | ADAM metallopeptidase domain 15 | 143537 | 163357 | 243364 | 24, 9, 45 |
| ADAM30 | ADAM metallopeptidase domain 30 | 134249 | 134193 | 134250 | 38, |
| ADAMTS4 | ADAM metallopeptidase with thrombospondin type 1 motif 4 | 158859 | 158850 | 158864 | 1, 42, 25 |
| ADAMTSL4 | ADAMTS like 4 | 143382 | 143369 | 143384 | 1, 32, 7 |
| AP4B1 | adaptor related protein complex 4 beta 1 subunit | 134262 | 188761 | 118655 | 39, 36, 22 |
| ADORA1 | adenosine A1 receptor | 163485 | 122180 | 133055 | |
| ADORA3 | adenosine A3 receptor | 282608 | 121933 | 116473 | 10, 26, 2 |
| ADAR | adenosine deaminase, RNA specific | 160710 | 160716 | 143603 | 40, 43, 22 |
| AMPD1 | adenosine monophosphate deaminase 1 | 116748 | 175984 | 213281 | 25, 15, 40 |
| AMPD2 | adenosine monophosphate deaminase 2 | 116337 | 134183 | 168765 | 8, 39, 3 |
| AHCYL1 | adenosylhomocysteinase like 1 | 168710 | 184371 | 143093 | 9, 26, 8 |
| ADCY10 | adenylate cyclase 10, soluble | 143199 | 197965 | 143158 | 38, 44, 14 |
| CAP1 | adenylate cyclase associated protein 1 | 131236 | 168389 | 131238 | 45, 43, 41 |
| AK2 | adenylate kinase 2 | 004455 | 116514 | 142920 | 29, 21, 3 |
| AK4 | adenylate kinase 4 | 162433 | 162434 | 116675 | 17, 1, 11 |
| AK5 | adenylate kinase 5 | 154027 | 142892 | 036549 | 10, 3, 1 |
| ADSS | adenylosuccinate synthase | 035687 | 173728 | 179397 | 45, 42, 4 |
| AJAP1 | adherens junctions associated protein 1 | 196581 | 198912 | 131697 | 10, 17, 36 |
| ADGRB2 | adhesion G protein-coupled receptor B2 | 121753 | 084636 | 134668 | 8, 42, 39 |
| ADGRL2 | adhesion G protein-coupled receptor L2 | 117114 | 162618 | 137941 | |
| ADGRL4 | adhesion G protein-coupled receptor L4 | 162618 | 137965 | 117114 | 45, 11, 10 |
| AMIGO1 | adhesion molecule with Ig like domain 1 | 181754 | 174151 | 156097 | 40, 42, 6 |
| ADIPOR1 | adiponectin receptor 1 | 159346 | 117153 | 159348 | 30, 9, 13 |
| ARF1 | ADP ribosylation factor 1 | 143761 | 154342 | 143793 | 3, 13, 32 |
| ARL8A | ADP ribosylation factor like GTPase 8A | 143862 | 170075 | 143851 | 33, 34, 5 |
| ADPRHL2 | ADP-ribosylhydrolase like 2 | 116863 | 092850 | 171812 | 43, 9, 26 |
| AGMAT | agmatinase | 116771 | 116138 | 197312 | 17, 3, 4 |
| AGRN | agrin | 188157 | 187608 | 237330 | 3, 27, 12 |
| AKIRIN1 | akirin 1 | 174574 | 158315 | 168653 | 34, 45, 31 |
| AKNAD1 | AKNA domain containing 1 | 162641 | 116266 | 274068 | 38, 10, 16 |
| AKT3 | AKT serine/threonine kinase 3 | 117020 | 054282 | 179456 | |
| ALDH4A1 | aldehyde dehydrogenase 4 family member A1 | 159423 | 255275 | 169991 | 39, 17, 24 |
| ALDH9A1 | aldehyde dehydrogenase 9 family member A1 | 143149 | 143198 | 143183 | 1, 40, 30 |
| AKR1A1 | aldo-keto reductase family 1 member A1 | 117448 | 117450 | 132780 | |
| AKR7L | aldo-keto reductase family 7 like (gene/pseudogene) | 211454 | 053372 | 162482 | 37, 27, 23 |
| AKR7A2 | aldo-keto reductase family 7 member A2 | 053371 | 162482 | 040487 | 34, 41, 9 |
| AKR7A3 | aldo-keto reductase family 7 member A3 | 162482 | 211454 | 053371 | 37, 16, 3 |
| ALG14 | ALG14, UDP-N-acetylglucosaminyltransferase subunit | 172339 | 117519 | 152078 | 10, 29, 20 |
| ALG6 | ALG6, alpha-1,3-glucosyltransferase | 088035 | 187140 | 142856 | 30, 4, 39 |
| ALPL | alkaline phosphatase, liver/bone/kidney | 162551 | 142794 | 076864 | 2, 1, 26 |
| A3GALT2 | alpha 1,3-galactosyltransferase 2 | 184389 | 160094 | 134686 | |
| ALX3 | ALX homeobox 3 | 156150 | 143093 | 186150 | 3, 16, 38 |
| AMY1A | amylase, alpha 1A (salivary) | 237763 | 243480 | 174876 | |
| AMY1B | amylase, alpha 1B (salivary) | 174876 | 237763 | 187733 | 34, 28, 44 |
| AMY1C | amylase, alpha 1C (salivary) | 187733 | 174876 | 198890 | 34, 28, 44 |
| AMY2A | amylase, alpha 2A (pancreatic) | 243480 | 240038 | 237763 | 28, 4, 34 |
| AMY2B | amylase, alpha 2B (pancreatic) | 240038 | 185946 | 243480 | 28, 10, 39 |
| AGL | amylo-alpha-1,6-glucosidase, 4-alpha-glucanotransferase | 162688 | 156869 | 283495 | 25, 22, 15 |
| APCS | amyloid P component, serum | 132703 | 184155 | 132693 | 19, 25, 20 |
| ANGEL2 | angel homolog 2 | 174606 | 143494 | 136643 | 9, 22, 42 |
| ANGPTL1 | angiopoietin like 1 | 116194 | 116191 | 116199 | 10, 44, 45 |
| ANGPTL3 | angiopoietin like 3 | 132855 | 116641 | 125703 | 19, 17, 26 |
| ANGPTL7 | angiopoietin like 7 | 171819 | 198793 | 120942 | 14, 1, 26 |
| AGTRAP | angiotensin II receptor associated protein | 177674 | 162490 | 215910 | 34, 9, 35 |
| AGT | angiotensinogen | 135744 | 135775 | 135773 | 19, 15, 8 |
| ASB17 | ankyrin repeat and SOCS box containing 17 | 154007 | 057468 | 184005 | 38, 8, |
| ANKRD13C | ankyrin repeat domain 13C | 118454 | 116754 | 197568 | 43, 10, 22 |
| ANKRD34A | ankyrin repeat domain 34A | 272031 | 271601 | 121851 | 14, 8, 6 |
| ANKRD35 | ankyrin repeat domain 35 | 198483 | 131788 | 143127 | 24, 13, 35 |
| ANKRD45 | ankyrin repeat domain 45 | 183831 | 162753 | 232113 | 38, 28, 8 |
| ANKRD65 | ankyrin repeat domain 65 | 235098 | 242485 | 205116 | 26, 14, 28 |
| ANXA9 | annexin A9 | 143412 | 143418 | 143409 | 16, 29, 19 |
| AZIN2 | antizyme inhibitor 2 | 142920 | 004455 | 116525 | 38, 8, 40 |
| APH1A | aph-1 homolog A, gamma-secretase subunit | 117362 | 118298 | 118292 | 9, 5, 39 |
| APITD1-CORT | APITD1 -CORT readthrough | 251503 | 142657 | 175279 | |
| APOA2 | apolipoprotein A2 | 158874 | 158869 | 158882 | 19, 36, 15 |
| APOBEC4 | apolipoprotein B mRNA editing enzyme catalytic polypeptide like 4 | 173627 | 143344 | 198756 | 38, 30, 24 |
| AQP10 | aquaporin 10 | 143595 | 143575 | 143515 | 37, 25, |
| ACAP3 | ArfGAP with coiled-coil, ankyrin repeat and PH domains 3 | 131584 | 162572 | 169972 | 26, 31, 3 |
| ASAP3 | ArfGAP with SH3 domain, ankyrin repeat and PH domain 3 | 088280 | 204219 | 007968 | 13, 29, 38 |
| RSRP1 | arginine and serine rich protein 1 | 117616 | 117614 | 187010 | 39, 4, 11 |
| AVPR1B | arginine vasopressin receptor 1B | 198049 | 263961 | 196550 | |
| RERE | arginine-glutamic acid dipeptide repeats | 142599 | 162426 | 074800 | |
| RNPEP | arginyl aminopeptidase | 176393 | 134375 | 163435 | 37, 26, 3 |
| AG01 | argonaute 1, RISC catalytic component | 092847 | 134698 | 126070 | 11, 40, 5 |
| AG03 | argonaute 3, RISC catalytic component | 126070 | 092847 | 092850 | 22, 3, 42 |
| AG04 | argonaute 4, RISC catalytic component | 134698 | 092853 | 092847 | 10, 29, 27 |
| ARTN | artemin | 117407 | 126091 | 117408 | 13, 29, 18 |
| ARV1 | ARV1 homolog, fatty acid homeostasis modulator | 173409 | 143643 | 182118 | 13, 34, 37 |
| ARNT | aryl hydrocarbon receptor nuclear translocator | 143437 | 143387 | 283324 | 18, 24, 4 |
| AADACL3 | arylacetamide deacetylase like 3 | 188984 | 204518 | 157330 | 35, 32 |
| AADACL4 | arylacetamide deacetylase like 4 | 204518 | 162496 | 188984 | |
| ASH1L | ASH1 like histone lysine methyltransferase | 116539 | 160753 | 125459 | 30, 22, 4 |
| DARS2 | aspartyl-tRNA synthetase 2, mitochondrial | 117593 | 120334 | 185278 | 31, 25, 42 |
| ASTN1 | astrotactin 1 | 152092 | 116183 | 198797 | 26, 14, 2 |
| ATXN7L2 | ataxin 7 like 2 | 162650 | 143028 | 174151 | 21, 38, 40 |
| AHCTF1 | AT-hook containing transcription factor 1 | 153207 | 143653 | 135747 | |
| AHDC1 | AT-hook DNA binding motif containing 1 | 126705 | 158195 | 000938 | 31, 27, 14 |
| ABCA4 | ATP binding cassette subfamily A member 4 | 198691 | 023909 | 137962 | 14, 25, 45 |
| ABCB10 | ATP binding cassette subfamily B member 10 | 135776 | 069248 | 135801 | 11, 29, 22 |
| ABCD3 | ATP binding cassette subfamily D member 3 | 117528 | 137962 | 117525 | 42, 39, 24 |
| ATPAF1 | ATP synthase mitochondrial F1 complex assembly factor 1 | 123472 | 142961 | 186118 | 27, 2, 23 |
| ATP5F1 | ATP synthase, H+ transporting, mitochondrial Fo complex subunit B1 | 116459 | 116455 | 143110 | 4, 34, 29 |
| AGBL4 | ATP/GTP binding protein like 4 | 186094 | 132122 | 162373 | 8, 17, 23 |
| ATP13A2 | ATPase 13A2 | 159363 | 117122 | 117118 | 7, 28, 22 |
| ATAD3A | ATPase family, AAA domain containing 3A | 197785 | 160072 | 205090 | 21, 35, 9 |
| ATAD3B | ATPase family, AAA domain containing 3B | 160072 | 215915 | 197785 | 31, 6, 3 |
| ATAD3C | ATPase family, AAA domain containing 3C | 215915 | 179403 | 160072 | 28, 17, 20 |
| ATP6V0B | ATPase H+ transporting V0 subunit b | 117410 | 132768 | 117411 | 31, 35, 14 |
| ATP6V1G3 | ATPase H+ transporting V1 subunit G3 | 151418 | 151414 | 081237 | 17, 25, 33 |
| ATPIF1 | ATPase inhibitory factor 1 | 130770 | 126698 | 130766 | 41, 25, 43 |
| ATP1A1 | ATPase Na+/K+ transporting subunit alpha 1 | 163399 | 173212 | 116815 | 40, 27, 35 |
| ATP1A2 | ATPase Na+/K+ transporting subunit alpha 2 | 018625 | 162729 | 132681 | 11, 8, 26 |
| ATP1A4 | ATPase Na+/K+ transporting subunit alpha 4 | 132681 | 018625 | 143318 | 38, 42, 4 |
| ATP1B1 | ATPase Na+/K+ transporting subunit beta 1 | 143153 | 143196 | 143156 | 29, 37, 17 |
| ATP8B2 | ATPase phospholipid transporting 8B2 | 143515 | 143595 | 160712 | 6, 31, 25 |
| ATP2B4 | ATPase plasma membrane Ca2+ transporting 4 | 058668 | 188770 | 122188 | 18, 44, 11 |
| ARID1A | AT-rich interaction domain 1A | 117713 | 117676 | 060642 | 43, 11, 23 |
| ARID4B | AT-rich interaction domain 4B | 054267 | 188739 | 152904 | 29, 22, 23 |
| ACKR1 | atypical chemokine receptor 1 (Duffy blood group) | 213088 | 162706 | 179639 | |
| AUNIP | aurora kinase A and ninein interacting protein | 127423 | 117640 | 182749 | 1, 13, 25 |
| AURKAIP1 | aurora kinase A interacting protein 1 | 175756 | 162576 | 221978 | 29, 15, 34 |
| ATG4C | autophagy related 4C cysteine peptidase | 125703 | 132855 | 187140 | 7, 10, 34 |
| AIDA | axin interactor, dorsalization associated | 186063 | 154305 | 162819 | 33, 45, 2 |
| AXDND1 | axonemal dynein light chain domain containing 1 | 162779 | 057252 | 116218 | 38, 40, 42 |
| BARHL2 | BarH like homeobox 2 | 143032 | 162664 | 122482 | |
| BSND | barttin CLCNK type accessory beta subunit | 162399 | 143001 | 169174 | 17, |
| BATF3 | basic leucine zipper ATF-like transcription factor 3 | 123685 | 162771 | 117697 | 10, 34, 39 |
| BLZF1 | basic leucine zipper nuclear factor 1 | 117475 | 143156 | 117477 | 45, 9, 18 |
| BTF3L4 | basic transcription factor 3 like 4 | 134717 | 198841 | 157077 | 4, 7, 5 |
| BCAS2 | BCAS2, pre-mRNA processing factor | 116752 | 197323 | 175984 | 4, 7, 34 |
| BCL10 | B-cell CLL/lymphoma 10 | 142867 | 162642 | 153904 | 45, 4, 22 |
| BCL9 | B-cell CLL/lymphoma 9 | 116128 | 131778 | 162836 | 2, 12, 26 |
| MCL1 | BCL2 family apoptosis regulator | 143384 | 143382 | 143420 | 42, 26, 44 |
| BNIPL | BCL2 interacting protein like | 163141 | 143363 | 143443 | 15, 30, 18 |
| BCL2L15 | BCL2 like 15 | 188761 | 134242 | 134262 | 4, 16, 3 |
| BECN2 | beclin 2 | 196289 | 174371 | 197769 | |
| BEND5 | BEN domain containing 5 | 162373 | 186094 | 162374 | 18, 10, 41 |
| BEST4 | bestrophin 4 | 142959 | 142937 | 173846 | 16, 9, 28 |
| B3GALT2 | beta-1,3-galactosyltransferase 2 | 162630 | 134371 | 162687 | 43, 8, 15 |
| B3GALT6 | beta-1,3-galactosyltransferase 6 | 176022 | 078808 | 184163 | 16, 22, 15 |
| B3GALNT2 | beta-1,3-N-acetylgalactosaminyltransferase 2 | 162885 | 116957 | 168243 | 30, 26, 6 |
| B4GALT2 | beta-1,4-galactosyltransferase 2 | 117411 | 117410 | 159214 | 3, 9, 18 |
| B4GALT3 | beta-1,4-galactosyltransferase 3 | 158850 | 143224 | 158859 | 13, 35, 9 |
| BRINP2 | BMP/retinoic acid inducible neural specific 2 | 198797 | 152092 | 120341 | 8, 42, 2 |
| BRINP3 | BMP/retinoic acid inducible neural specific 3 | 162670 | 116711 | 150681 | 8, 10, 19 |
| BOLA1 | bolA family member 1 | 178096 | 184270 | 159164 | 22, 15, 32 |
| BGLAP | bone gamma-carboxyglutamate protein | 242252 | 160783 | 160781 | 29, 6, 15 |
| BMP8A | bone morphogenetic protein 8a | 183682 | 127603 | 090621 | 6, 40, 18 |
| BMP8B | bone morphogenetic protein 8b | 116985 | 084072 | 198754 | 35, 3, 7 |
| BCAR3 | breast cancer anti-estrogen resistance 3 | 137936 | 137942 | 067334 | 1, 32, 23 |
| BCAN | brevican | 132692 | 132702 | 132688 | 8, 11, 14 |
| BROX | BRO1 domain and CAAX motif containing | 162819 | 186063 | 197520 | 4, 42, 25 |
| BRDT | bromodomain testis associated | 137948 | 069702 | 172031 | 38, 24, 25 |
| BSDC1 | BSD domain containing 1 | 160058 | 225828 | 254553 | 26, 39, 2 |
| BTBD19 | BTB domain containing 19 | 222009 | 188396 | 117425 | 40, 22, 27 |
| BTBD8 | BTB domain containing 8 | 189195 | 230667 | 069712 | 7, 25, 23 |
| BTG2 | BTG anti-proliferation factor 2 | 159388 | 133063 | 122176 | 21, 40, 22 |
| C2CD4D | C2 calcium dependent domain containing 4D | 225556 | 143365 | 196407 | 23, 16, 8 |
| CACHD1 | cache domain containing 1 | 158966 | 177414 | 162437 | 24, 12, 29 |
| CELSR2 | cadherin EGF LAG seven-pass G-type receptor 2 | 143126 | 031698 | 134222 | 29, 23, 8 |
| CACNA1E | calcium voltage-gated channel subunit alpha1 E | 198216 | 162783 | 179930 | 8, 20, 40 |
| CACNA1S | calcium voltage-gated channel subunit alpha1 S | 081248 | 116852 | 232237 | 25, 18, 40 |
| CAMK1G | calcium/calmodulin dependent protein kinase IG | 008118 | 076356 | 196878 | 34, 8, 17 |
| CAMK2N1 | calcium/calmodulin dependent protein kinase II inhibitor 1 | 162545 | 158816 | 090432 | 29, 33, 43 |
| CACYBP | calcyclin binding protein | 116161 | 203737 | 120333 | 4, 7, 26 |
| CAMTA1 | calmodulin binding transcription activator 1 | 171735 | 007923 | 049245 | 14, 8, 25 |
| CALML6 | calmodulin like 6 | 169885 | 078369 | 178821 | 26, 37, 25 |
| CAMSAP2 | calmodulin regulated spectrin associated protein family member 2 | 118200 | 118197 | 170128 | 4, 9, 32 |
| CAPN2 | calpain 2 | 162909 | 203697 | 143514 | 24, 42, 1 |
| CAPN8 | calpain 8 | 203697 | 178395 | 162909 | 37, 3, 18 |
| CAPN9 | calpain 9 | 135773 | 135744 | 119280 | 42, 3, 30 |
| CNN3 | calponin 3 | 117519 | 143036 | 172339 | 43, 13, 2 |
| CASQ1 | calsequestrin 1 | 143318 | 132681 | 162734 | 25, 15, 18 |
| CASQ2 | calsequestrin 2 | 118729 | 173218 | 177551 | 15, 43, 25 |
| CLSTN1 | calsyntenin 1 | 171603 | 171608 | 280113 | 18, 21, 26 |
| CREB3L4 | cAMP responsive element binding protein 3 like 4 | 143578 | 143570 | 143543 | 33, 26, 30 |
| CNR2 | cannabinoid receptor 2 | 188822 | 179163 | 189266 | 22, 7, 5 |
| CAPZA1 | capping actin protein of muscle Z-line alpha subunit 1 | 116489 | 007341 | 155363 | 4, 45, 3 |
| CAPZB | capping actin protein of muscle Z-line beta subunit | 077549 | 040487 | 173436 | 40, 34, 5 |
| CA14 | carbonic anhydrase 14 | 118298 | 143401 | 117362 | 11, 12, 35 |
| CA6 | carbonic anhydrase 6 | 131686 | 074800 | 197241 | 34, 30, 23 |
| CPT2 | carnitine palmitoyltransferase 2 | 157184 | 162383 | 162384 | 31, 36, 17 |
| CASP9 | caspase 9 | 132906 | 215704 | 116138 | 9, 21, 1 |
| CASZ1 | castor zinc finger 1 | 130940 | 142655 | 175262 | |
| CTNNBIP1 | catenin beta interacting protein 1 | 178585 | 280113 | 162441 | 41, 20, 35 |
| CTSE | cathepsin E | 196188 | 276600 | 263961 | 37, 3, 30 |
| CTSK | cathepsin K | 143387 | 163131 | 143437 | |
| CTSS | cathepsin S | 163131 | 143452 | 143387 | 5, 36, 10 |
| CATSPER4 | cation channel sperm associated 4 | 188782 | 142675 | 130695 | 38, |
| CITED4 | Cbp/p300 interacting transactivator with Glu/Asp rich carboxy-terminal domain 4 | 179862 | 117013 | 171793 | 22, 4, 28 |
| CD101 | CD101 molecule | 134256 | 134247 | 116830 | 18, 42, 7 |
| CD160 | CD160 molecule | 117281 | 174827 | 265491 | 5, 39, 28 |
| CD164L2 | CD164 molecule like 2 | 174950 | 142748 | 181773 | 7, 27, 39 |
| CD1A | CD1a molecule | 158477 | 158473 | 158481 | 39, 5, 22 |
| CD1B | CD1b molecule | 158485 | 158481 | 158488 | 39, 30, 15 |
| CD1C | CD1c molecule | 158481 | 158477 | 158485 | 39, 45, 15 |
| CD1D | CD1d molecule | 158473 | 183853 | 158477 | 39, 22, 9 |
| CD1E | CD1e molecule | 158488 | 158485 | 186306 | 39, 5, 15 |
| CD2 | CD2 molecule | 116824 | 143061 | 134247 | 30, 39, 36 |
| CD244 | CD244 molecule | 122223 | 122224 | 179914 | 36, 5, 39 |
| CD247 | CD247 molecule | 198821 | 143190 | 143162 | |
| CD34 | CD34 molecule | 174059 | 117335 | 076356 | 15, 45, 36 |
| CD46 | CD46 molecule | 117335 | 197721 | 174059 | 29, 42, 32 |
| CD48 | CD48 molecule | 117091 | 117090 | 026751 | 21, 39, 41 |
| CD5L | CD5 molecule like | 073754 | 163534 | 183853 | 36, 28, 32 |
| CD52 | CD52 molecule | 169442 | 158062 | 176092 | 30, 5, 2 |
| CD53 | CD53 molecule | 143119 | 177272 | 121931 | 30, 36, 22 |
| CD55 | CD55 molecule (Cromer blood group) | 196352 | 123838 | 117322 | 9, 45, 42 |
| CD58 | CD58 molecule | 116815 | 163399 | 143061 | 37, 13, 1 |
| CD84 | CD84 molecule | 066294 | 162739 | 117090 | 36, 5, 22 |
| CLK2 | CDC like kinase 2 | 176444 | 116521 | 143630 | 27, 1, 12 |
| CKS1B | CDC28 protein kinase regulatory subunit 1B | 173207 | 160691 | 160688 | 3, 29, 21 |
| CDC42BPA | CDC42 binding protein kinase alpha | 143776 | 163050 | 181450 | 26, 14, 33 |
| CDC42SE1 | CDC42 small effector 1 | 197622 | 143443 | 213190 | 18, 5, 39 |
| CADM3 | cell adhesion molecule 3 | 162706 | 163568 | 213088 | 31, 8, 32 |
| CDC14A | cell division cycle 14A | 079335 | 137996 | 181656 | 38, 29, 45 |
| CDC20 | cell division cycle 20 | 117399 | 117400 | 066322 | 21, 3, 27 |
| CDC42 | cell division cycle 42 | 070831 | 142789 | 162552 | 45, 29, 3 |
| CDC7 | cell division cycle 7 | 097046 | 162669 | 069702 | 12, 22, 39 |
| CDC73 | cell division cycle 73 | 134371 | 023572 | 162630 | 26, 7, 33 |
| CDCA8 | cell division cycle associated 8 | 134690 | 116922 | 183317 | 21, 22, 39 |
| CREG1 | cellular repressor of E1A stimulated genes 1 | 143162 | 198821 | 198771 | 2, 4, 26 |
| CRABP2 | cellular retinoic acid binding protein 2 | 143320 | 132688 | 143319 | 14, 27, 28 |
| CCSAP | centriole, cilia and spindle associated protein | 154429 | 213029 | 143632 | 32, 24, 26 |
| CENPF | centromere protein F | 117724 | 152104 | 082482 | 19, 13, 15 |
| CENPL | centromere protein L | 120334 | 076321 | 117593 | 45, 9, 39 |
| CENPS | centromere protein S | 175279 | 251503 | 241563 | |
| CEP104 | centrosomal protein 104 | 116198 | 130764 | 169598 | 29, 33, 27 |
| CEP170 | centrosomal protein 170 | 143702 | 180287 | 054282 | |
| CEP350 | centrosomal protein 350 | 135837 | 143337 | 116260 | 22, 11, 43 |
| CEP85 | centrosomal protein 85 | 130695 | 188782 | 142669 | 38, 42, 39 |
| CERS2 | ceramide synthase 2 | 143418 | 143379 | 143412 | 9, 1, 29 |
| CPTP | ceramide-1-phosphate transfer protein | 224051 | 127054 | 169962 | 28, 1, 38 |
| CCT3 | chaperonin containing TCP1 subunit 3 | 163468 | 189030 | 163467 | 21, 13, 9 |
| CHIT1 | chitinase 1 | 133063 | 133048 | 159388 | |
| CHI3L1 | chitinase 3 like 1 | 133048 | 133055 | 133063 | 10, 5, 13 |
| CHI3L2 | chitinase 3 like 2 | 064886 | 162777 | 134216 | 10, 3, 21 |
| CHIA | chitinase, acidic | 134216 | 064886 | 173947 | 40, 37, 44 |
| CTBS | chitobiase | 117151 | 174021 | 117155 | 41, 29, 1 |
| CLCA1 | chloride channel accessory 1 | 016490 | 137975 | 016602 | 16, 4, 37 |
| CLCA2 | chloride channel accessory 2 | 137975 | 122417 | 016490 | 24, 42, 13 |
| CLCA4 | chloride channel accessory 4 | 016602 | 016490 | 097033 | 13, 42, 4 |
| CLCC1 | chloride channel CLIC like 1 | 121940 | 121957 | 085433 | 13, 7, 33 |
| CLIC4 | chloride intracellular channel 4 | 169504 | 133226 | 020633 | 45, 42, 44 |
| CLCN6 | chloride voltage-gated channel 6 | 011021 | 177000 | 175206 | 26, 29, 1 |
| CLCNKA | chloride voltage-gated channel Ka | 186510 | 173641 | 184908 | 17, 29, 31 |
| CLCNKB | chloride voltage-gated channel Kb | 184908 | 186510 | 185519 | 17, 29, 40 |
| CHML | CHM like, Rab escort protein 2 | 203668 | 054277 | 162843 | 34, 40, 6 |
| CEPT1 | choline/ethanolamine phosphotransferase 1 | 134255 | 156171 | 162777 | 40, 22, 11 |
| CHRM3 | cholinergic receptor muscarinic 3 | 133019 | 270188 | 155816 | 28, |
| CHRNB2 | cholinergic receptor nicotinic beta 2 subunit | 160716 | 160714 | 160710 | 26, 8, 15 |
| CHTOP | chromatin target of PRMT1 | 160679 | 160678 | 143553 | 36, 19, 24 |
| CHD1L | chromodomain helicase DNA binding protein 1 like | 131778 | 131781 | 116128 | 25, 31, 12 |
| CHD5 | chromodomain helicase DNA binding protein 5 | 116254 | 069424 | 116251 | 8, 38, 14 |
| C1orf100 | chromosome 1 open reading frame 100 | 173728 | 179456 | 035687 | 38, 23, |
| C1orf101 | chromosome 1 open reading frame 101 | 179397 | 035687 | 121644 | |
| C1orf105 | chromosome 1 open reading frame 105 | 180999 | 135845 | 094975 | 15, 24, 38 |
| C1orf106 | chromosome 1 open reading frame 106 | 163362 | 170128 | 116852 | 42, 24, 3 |
| C1orf109 | chromosome 1 open reading frame 109 | 116922 | 169218 | 134690 | 31, 13, 44 |
| C1orf111 | chromosome 1 open reading frame 111 | 171722 | 239887 | 198574 | 38, 5, 8 |
| C1orf112 | chromosome 1 open reading frame 112 | 000460 | 174175 | 188404 | 29, 39, 9 |
| C1orf115 | chromosome 1 open reading frame 115 | 162817 | 116141 | 117791 | 32, 40, 18 |
| C1orf116 | chromosome 1 open reading frame 116 | 182795 | 162897 | 123836 | 42, 24, 33 |
| C1orf122 | chromosome 1 open reading frame 122 | 197982 | 196449 | 188786 | 31, 34, 28 |
| C1orf123 | chromosome 1 open reading frame 123 | 162384 | 157184 | 162385 | 26, 31, 1 |
| C1orf127 | chromosome 1 open reading frame 127 | 175262 | 130940 | 120948 | 28, 25, 10 |
| C1orf131 | chromosome 1 open reading frame 131 | 143633 | 119283 | 116906 | 29, 26, 7 |
| C1orf141 | chromosome 1 open reading frame 141 | 203963 | 116704 | 162594 | 38, 20, |
| C1orf145 | chromosome 1 open reading frame 145 | 162913 | 181873 | 154358 | 39, 38, 20 |
| C1orf146 | chromosome 1 open reading frame 146 | 203910 | 069712 | 174842 | 38, 5, 17 |
| C1orf158 | chromosome 1 open reading frame 158 | 157330 | 188984 | 116726 | 38, 20, 6 |
| C1orf159 | chromosome 1 open reading frame 159 | 131591 | 237330 | 162571 | 9, 39, 27 |
| C1orf162 | chromosome 1 open reading frame 162 | 143110 | 116459 | 121933 | 30, 22, 10 |
| C1orf167 | chromosome 1 open reading frame 167 | 215910 | 177674 | 177000 | 18, 5, 32 |
| C1orf168 | chromosome 1 open reading frame 168 | 187889 | 162409 | 157131 | 10, 18, 4 |
| C1orf174 | chromosome 1 open reading frame 174 | 198912 | 169598 | 196581 | 3, 19, 9 |
| C1orf185 | chromosome 1 open reading frame 185 | 204006 | 123080 | 123091 | 38, |
| C1orf186 | chromosome 1 open reading frame 186 | 263961 | 196188 | 198049 | 44, 17, 27 |
| C1orf189 | chromosome 1 open reading frame 189 | 163263 | 143549 | 143612 | 30, 38, |
| C1orf194 | chromosome 1 open reading frame 194 | 179902 | 215717 | 116299 | 38, 10, 14 |
| C1orf198 | chromosome 1 open reading frame 198 | 119280 | 135773 | 143643 | 11, 43, 26 |
| C1orf204 | chromosome 1 open reading frame 204 | 188004 | 158714 | 243284 | 2, 30, 40 |
| C1orf21 | chromosome 1 open reading frame 21 | 116667 | 198860 | 116406 | 2, 33, 10 |
| C1orf210 | chromosome 1 open reading frame 210 | 253313 | 179178 | 066056 | 22, 40, 42 |
| C1orf216 | chromosome 1 open reading frame 216 | 142686 | 126067 | 092853 | 34, 8, 33 |
| C1orf226 | chromosome 1 open reading frame 226 | 239887 | 254706 | 171722 | 42, 4, 24 |
| C1orf228 | chromosome 1 open reading frame 228 | 198520 | 126106 | 142945 | |
| C1orf234 | chromosome 1 open reading frame 234 | 227868 | 215906 | 004487 | 38, 32, |
| C1orf27 | chromosome 1 open reading frame 27 | 157181 | 047410 | 279374 | 29, 7, 19 |
| C1orf35 | chromosome 1 open reading frame 35 | 143793 | 143761 | 162910 | 41, 21, 3 |
| C1orf43 | chromosome 1 open reading frame 43 | 143612 | 163263 | 143569 | 9, 6, 18 |
| C1orf50 | chromosome 1 open reading frame 50 | 164008 | 117385 | 283580 | 29, 21, 17 |
| C1orf52 | chromosome 1 open reading frame 52 | 162642 | 097096 | 142867 | 15, 44, 35 |
| C1orf53 | chromosome 1 open reading frame 53 | 203724 | 213047 | 143355 | 44, 6, 10 |
| C1orf54 | chromosome 1 open reading frame 54 | 118292 | 117362 | 159208 | 15, 18, 1 |
| C1orf56 | chromosome 1 open reading frame 56 | 143443 | 163141 | 197622 | 38, 9, 13 |
| C1orf61 | chromosome 1 open reading frame 61 | 125462 | 132677 | 116604 | 8, 14, 31 |
| C1orf64 | chromosome 1 open reading frame 64 | 183888 | 116809 | 173641 | 31, 23, 17 |
| C1orf68 | chromosome 1 open reading frame 68 | 198854 | 187170 | 203786 | 35, 10, |
| C1orf74 | chromosome 1 open reading frame 74 | 162757 | 009790 | 117595 | 7, 30, 42 |
| C1orf87 | chromosome 1 open reading frame 87 | 162598 | 134716 | 162599 | 30, 38, 20 |
| C1orf94 | chromosome 1 open reading frame 94 | 142698 | 176256 | 163866 | 38, 25, 12 |
| CTRC | chymotrypsin C | 162438 | 142634 | 142615 | 28, 4, 36 |
| CELA2A | chymotrypsin like elastase family member 2A | 142615 | 162438 | 215704 | 28, 25, 38 |
| CELA2B | chymotrypsin like elastase family member 2B | 215704 | 142615 | 132906 | 28, 4, 20 |
| CELA3A | chymotrypsin like elastase family member 3A | 142789 | 219073 | 070831 | 28, 4, 19 |
| CELA3B | chymotrypsin like elastase family member 3B | 219073 | 142798 | 142789 | 28, 4, 19 |
| CFAP126 | cilia and flagella associated protein 126 | 188931 | 143252 | 143226 | |
| CFAP45 | cilia and flagella associated protein 45 | 213085 | 256029 | 158710 | 38, 20, 30 |
| CFAP57 | cilia and flagella associated protein 57 | 243710 | 117395 | 179178 | 30, 38, |
| CFAP74 | cilia and flagella associated protein 74 | 142609 | 178821 | 187730 | |
| CROCC | ciliary rootlet coiled-coil, rootletin | 058453 | 279132 | 117122 | 26, 12, 6 |
| CGN | cingulin | 143375 | 143442 | 143367 | 42, 16, 28 |
| CIART | circadian associated repressor of transcription | 159208 | 118292 | 266472 | 29, 23, 4 |
| CLSPN | claspin | 092853 | 142686 | 134698 | 18, 23, 3 |
| CLDN19 | claudin 19 | 164007 | 065978 | 117385 | 17, 14, 32 |
| CPSF3L | cleavage and polyadenylation specific factor 3-like | 127054 | 169972 | 224051 | 39, 1, 21 |
| F3 | coagulation factor III, tissue factor | 117525 | 117528 | 143036 | 13, 42, 24 |
| F5 | coagulation factor V | 198734 | 117479 | 174175 | 32, 19, 5 |
| F13B | coagulation factor XIIIB chain | 143278 | 134389 | 066279 | 19, 15, 10 |
| COPA | coatomer protein complex subunit alpha | 122218 | 162735 | 162736 | |
| COQ8A | coenzyme Q8A | 163050 | 143801 | 143776 | 24, 15, 1 |
| CC2D1B | coiled-coil and C2 domain containing 1B | 154222 | 157077 | 085840 | |
| CCDC163 | coiled-coil domain containing 163 | 280670 | 070759 | 132763 | 18, 42, 30 |
| CCDC17 | coiled-coil domain containing 17 | 159588 | 132780 | 159592 | 42, 20, 15 |
| CCDC18 | coiled-coil domain containing 18 | 122483 | 117500 | 117505 | 22, 31, 34 |
| CCDC181 | coiled-coil domain containing 181 | 117477 | 117475 | 117479 | |
| CCDC185 | coiled-coil domain containing 185 | 178395 | 143502 | 203697 | 38, 23, 20 |
| CCDC190 | coiled-coil domain containing 190 | 185860 | 132196 | 117152 | 24, 42, 17 |
| CCDC24 | coiled-coil domain containing 24 | 159214 | 117411 | 196517 | 1, 22, 29 |
| CCDC27 | coiled-coil domain containing 27 | 162592 | 078900 | 235169 | 38, |
| CCDC28B | coiled-coil domain containing 28B | 160050 | 084652 | 160051 | 29, 21, 15 |
| CCDC30 | coiled-coil domain containing 30 | 186409 | 127125 | 171960 | 31, 2, 17 |
| CSDE1 | cold shock domain containing E1 | 009307 | 213281 | 052723 | 42, 39, 24 |
| COLGALT2 | collagen beta(1-O)galactosyltransferase 2 | 198756 | 173627 | 198860 | 42, 8, 31 |
| COL9A2 | collagen type IX alpha 2 chain | 049089 | 084073 | 084070 | 11, 21, 14 |
| COL8A2 | collagen type VIII alpha 2 chain | 171812 | 116863 | 054116 | 16, 6, 14 |
| COL11A1 | collagen type XI alpha 1 chain | 060718 | 118733 | 185946 | 11, 6, 2 |
| COL16A1 | collagen type XVI alpha 1 chain | 084636 | 162517 | 121753 | 26, 6, 31 |
| COL24A1 | collagen type XXIV alpha 1 chain | 171502 | 117174 | 122417 | 11, 7, 37 |
| CSF1 | colony stimulating factor 1 | 184371 | 198758 | 168710 | 3, 42, 28 |
| CSF3R | colony stimulating factor 3 receptor | 119535 | 116898 | 163873 | 32, 5, 36 |
| C1QA | complement C1q A chain | 173372 | 070886 | 159189 | 36, 13, 16 |
| C1QB | complement C1q B chain | 173369 | 159189 | 133216 | 36, 1, 2 |
| C1QC | complement C1q C chain | 159189 | 173372 | 173369 | 36, 20, 32 |
| CR1 | complement C3b/C4b receptor 1 (Knops blood group) | 203710 | 117322 | 197721 | 22, 26, 45 |
| CR1L | complement C3b/C4b receptor 1 like | 197721 | 203710 | 117335 | 37, 7, 45 |
| CR2 | complement C3d receptor 2 | 117322 | 196352 | 203710 | 22, 36, 39 |
| C8A | complement C8 alpha chain | 157131 | 187889 | 021852 | 19, 36, 5 |
| C8B | complement C8 beta chain | 021852 | 157131 | 173406 | 19, 20, |
| C4BPA | complement component 4 binding protein alpha | 123838 | 123843 | 196352 | 19, 4, 20 |
| C4BPB | complement component 4 binding protein beta | 123843 | 180667 | 123838 | 19, 9, 16 |
| CFH | complement factor H | 000971 | 162687 | 116785 | 42, 19, 10 |
| CFHR1 | complement factor H related 1 | 244414 | 276911 | 134365 | 19, 15, 36 |
| CFHR2 | complement factor H related 2 | 080910 | 134365 | 134389 | 19, 10, |
| CFHR3 | complement factor H related 3 | 116785 | 000971 | 276911 | 19, 18, 25 |
| CFHR4 | complement factor H related 4 | 134365 | 244414 | 080910 | 19, |
| CFHR5 | complement factor H related 5 | 134389 | 080910 | 143278 | 19, |
| COG2 | component of oligomeric golgi complex 2 | 135775 | 177614 | 135744 | 24, 29, 38 |
| CNKSR1 | connector enhancer of kinase suppressor of Ras 1 | 142675 | 142684 | 188782 | 13, 24, 32 |
| CNST | consortin, connexin sorting protein | 162852 | 162851 | 143653 | 42, 1, 24 |
| CNTN2 | contactin 2 | 184144 | 163531 | 174529 | |
| CNIH3 | cornichon family AMPA receptor auxiliary protein 3 | 143786 | 162923 | 185842 | 35, 45, 8 |
| CNIH4 | cornichon family AMPA receptor auxiliary protein 4 | 143771 | 143748 | 162923 | 1, 29, 10 |
| CRNN | cornulin | 143536 | 143520 | 186207 | 13, 9, 30 |
| CORT | cortistatin | 241563 | 175279 | 160049 | 31, 38, 9 |
| COX20 | COX20, cytochrome c oxidase assembly factor | 203667 | 121644 | 153187 | 2, 19, 29 |
| CRP | C-reactive protein | 132693 | 132703 | 158716 | |
| CRTC2 | CREB regulated transcription coactivator 2 | 160741 | 198837 | 143570 | 40, 26, 6 |
| CRB1 | crumbs 1, cell polarity complex component | 134376 | 177888 | 213047 | 38, 26, 10 |
| CRYZ | crystallin zeta | 116791 | 178965 | 162623 | 4, 31, 19 |
| CTPS1 | CTP synthase 1 | 171793 | 179862 | 171790 | 34, 21, 45 |
| CTTNBP2NL | CTTNBP2 N-terminal like | 143079 | 171385 | 134245 | 24, 29, 26 |
| CSMD2 | CUB and Sushi multiple domains 2 | 121904 | 121903 | 176256 | 38, 8, 25 |
| CDCP2 | CUB domain containing protein 2 | 157211 | 256407 | 215883 | |
| CELF3 | CUGBP, Elav-like family member 3 | 159409 | 143376 | 178796 | 28, 14, 8 |
| CDK11A | cyclin dependent kinase 11A | 008128 | 189339 | 215790 | 18, 39, 1 |
| CDK11B | cyclin dependent kinase 11B | 248333 | 189409 | 189339 | 21, 27, 18 |
| CDK18 | cyclin dependent kinase 18 | 117266 | 186007 | 174514 | 8, 4, 14 |
| CDKN2C | cyclin dependent kinase inhibitor 2C | 123080 | 185104 | 204006 | 39, 34, 7 |
| CCNL2 | cyclin L2 | 221978 | 175756 | 242485 | 33, 30, 13 |
| CTH | cystathionine gamma-lyase | 116761 | 279230 | 050628 | 19, 14, 4 |
| CSRP1 | cysteine and glycine rich protein 1 | 159176 | 174307 | 134369 | 43, 13, 44 |
| CYR61 | cysteine rich angiogenic inducer 61 | 142871 | 153904 | 117174 | 1, 26, 45 |
| CRCT1 | cysteine rich C-terminal 1 | 169509 | 279850 | 185966 | 15, 30, 35 |
| CDA | cytidine deaminase | 158825 | 183114 | 158828 | 5, 19, 10 |
| CMPK1 | cytidine/uridine monophosphate kinase 1 | 162368 | 123473 | 186790 | 4, 24, 45 |
| CYB5R1 | cytochrome b5 reductase 1 | 159348 | 159346 | 163444 | 29, 18, 32 |
| CYB5RL | cytochrome b5 reductase like | 215883 | 157211 | 116221 | 1, 6, 22 |
| CYB561D1 | cytochrome b561 family member D1 | 174151 | 162650 | 181754 | 39, 27, 18 |
| COA6 | cytochrome c oxidase assembly factor 6 | 168275 | 183780 | 059588 | 29, 3, 28 |
| COA7 | cytochrome c oxidase assembly factor 7 (putative) | 162377 | 182183 | 162378 | |
| CYP2J2 | cytochrome P450 family 2 subfamily J member 2 | 134716 | 134709 | 162598 | 19, 10, 32 |
| CYP4A11 | cytochrome P450 family 4 subfamily A member 11 | 187048 | 142973 | 186377 | 19, 17, 5 |
| CYP4A22 | cytochrome P450 family 4 subfamily A member 22 | 162365 | 186160 | 162366 | |
| CYP4B1 | cytochrome P450 family 4 subfamily B member 1 | 142973 | 159658 | 187048 | |
| CYP4X1 | cytochrome P450 family 4 subfamily X member 1 | 186377 | 187048 | 186160 | 42, 26, 45 |
| CYP4Z1 | cytochrome P450 family 4 subfamily Z member 1 | 186160 | 186377 | 162365 | 10, 23, 42 |
| DAB1 | DAB1, reelin adaptor protein | 173406 | 021852 | 162600 | 10, 8, 12 |
| DCST1 | DC-STAMP domain containing 1 | 163357 | 163354 | 143537 | 26, 38, 14 |
| DCST2 | DC-STAMP domain containing 2 | 163354 | 160685 | 163357 | 38, 39, 14 |
| DCAF6 | DDB1 and CUL4 associated factor 6 | 143164 | 143158 | 143147 | 38, 40, 24 |
| DCAF8 | DDB1 and CUL4 associated factor 8 | 132716 | 162734 | 258465 | 31, 24, 39 |
| DDX20 | DEAD-box helicase 20 | 064703 | 197852 | 171385 | 38, 22, 45 |
| DDX59 | DEAD-box helicase 59 | 118197 | 118193 | 118200 | 9, 31, 22 |
| DAP3 | death associated protein 3 | 132676 | 163374 | 116580 | 31, 24, 43 |
| DEDD | death effector domain containing | 158796 | 158793 | 143222 | 9, 38, 22 |
| DOCK7 | dedicator of cytokinesis 7 | 116641 | 162607 | 132855 | |
| DHDDS | dehydrodolichyl diphosphate synthase subunit | 117682 | 131914 | 198830 | |
| DHRS3 | dehydrogenase/reductase 3 | 162496 | 048707 | 204518 | 1, 18, 9 |
| DEGS1 | delta 4-desaturase, sphingolipid 1 | 143753 | 143756 | 143748 | 45, 4, 2 |
| DENND1B | DENN domain containing 1B | 213047 | 134376 | 203724 | 4, 29, 22 |
| DENND2C | DENN domain containing 2C | 175984 | 116752 | 116748 | 18, 38, 25 |
| DENND2D | DENN domain containing 2D | 162777 | 134255 | 064886 | 36, 39, 3 |
| DENND4B | DENN domain containing 4B | 198837 | 143614 | 160741 | 5, 41, 22 |
| DTL | denticleless E3 ubiquitin protein ligase homolog | 143476 | 143493 | 066027 | 5, 38, 21 |
| DNTTIP2 | deoxynucleotidyltransferase terminal interacting protein 2 | 067334 | 137936 | 023909 | 45, 18, 28 |
| DNASE2B | deoxyribonuclease 2 beta | 137976 | 203943 | 117133 | 20, 35, 25 |
| DEPDC1 | DEP domain containing 1 | 024526 | 116745 | 033122 | 13, 22, 43 |
| DPT | dermatopontin | 143196 | 143184 | 143153 | 1, 30, 27 |
| DESI2 | desumoylating isopeptidase 2 | 121644 | 179397 | 203667 | 22, 4, 15 |
| DHX9 | DExH-box helicase 9 | 135829 | 135838 | 157060 | 42, 38, 12 |
| DMBX1 | diencephalon/mesencephalon homeobox 1 | 197587 | 117480 | 282881 | |
| DIEXF | digestive organ expansion factor homolog (zebrafish) | 117597 | 117595 | 143469 | 45, 9, 36 |
| DBT | dihydrolipoamide branched chain transacylase E2 | 137992 | 122477 | 137996 | 31, 40, 1 |
| DPYD | dihydropyrimidine dehydrogenase | 188641 | 117569 | 162627 | 26, 22, 42 |
| DDAH1 | dimethylarginine dimethylaminohydrolase 1 | 153904 | 142867 | 142871 | 17, 4, 45 |
| DPH5 | diphthamide biosynthesis 5 | 117543 | 162695 | 170989 | 18, 31, 9 |
| DIRAS3 | DIRAS family GTPase 3 | 162595 | 172380 | 116729 | 31, 34, 7 |
| DDR2 | discoidin domain receptor tyrosine kinase 2 | 162733 | 117143 | 132196 | |
| DVL1 | dishevelled segment polarity protein 1 | 107404 | 169962 | 162576 | 1, 34, 6 |
| DISP1 | dispatched RND transporter family member 1 | 154309 | 197520 | 187554 | 43, 13, 38 |
| DISP3 | dispatched RND transporter family member 3 | 204624 | 120942 | 116661 | 14, 38, 20 |
| DISC1 | disrupted in schizophrenia 1 | 162946 | 116918 | 116991 | 30, 43, 19 |
| DLGAP3 | DLG associated protein 3 | 116544 | 187513 | 271741 | 38, 36, 14 |
| DMRTA2 | DMRT like family A2 | 142700 | 162374 | 185104 | 14, 25, 8 |
| DMRTB1 | DMRT like family B with proline rich C-terminal 1 | 143006 | 157193 | 174332 | 38, 32, 8 |
| DCLRE1B | DNA cross-link repair 1B | 118655 | 134262 | 163349 | 6, 9, 39 |
| DDI2 | DNA damage inducible 1 homolog 2 | 197312 | 116771 | 215695 | 40, 24, 18 |
| DRAM2 | DNA damage regulated autophagy modulator 2 | 156171 | 121931 | 134255 | 1, 34, 7 |
| DFFA | DNA fragmentation factor subunit alpha | 160049 | 241563 | 142655 | 41, 25, 21 |
| DFFB | DNA fragmentation factor subunit beta | 169598 | 116198 | 198912 | 10, 45, 24 |
| DMAP1 | DNA methyltransferase 1 associated protein 1 | 178028 | 283039 | 117419 | 9, 2, 21 |
| DNAJB4 | DnaJ heat shock protein family (Hsp40) member B4 | 162616 | 162613 | 137960 | 45, 13, 26 |
| DNAJC11 | DnaJ heat shock protein family (Hsp40) member C11 | 007923 | 041988 | 171735 | 21, 41, 1 |
| DNAJC16 | DnaJ heat shock protein family (Hsp40) member C16 | 116138 | 132906 | 116771 | 22, 26, 7 |
| DNAJC6 | DnaJ heat shock protein family (Hsp40) member C6 | 116675 | 162433 | 213625 | 26, 8, 31 |
| DNAJC8 | DnaJ heat shock protein family (Hsp40) member C8 | 126698 | 169403 | 130770 | 29, 30, 3 |
| DDOST | dolichyl-diphosphooligosaccharide-protein glycosyltransferase non-catalytic subunit | 244038 | 158828 | 117245 | 13, 30, 4 |
| DPM3 | dolichyl-phosphate mannosyltransferase subunit 3 | 179085 | 169241 | 163463 | 31, 33, 25 |
| DRAXIN | dorsal inhibitory axon guidance protein | 162490 | 116670 | 177674 | |
| DCDC2B | doublecortin domain containing 2B | 222046 | 160051 | 160055 | 20, 44, |
| DR1 | down-regulator of transcription 1 | 117505 | 122483 | 137942 | 39, 22, 34 |
| DPH2 | DPH2 homolog | 132768 | 117408 | 117410 | 21, 9, 3 |
| DSTYK | dual serine/threonine and tyrosine protein kinase | 133059 | 117222 | 133069 | 35, 14, 11 |
| DUSP10 | dual specificity phosphatase 10 | 143507 | 136630 | 143512 | 6, 4, 26 |
| DUSP12 | dual specificity phosphatase 12 | 081721 | 162746 | 118217 | 3, 23, 44 |
| DUSP23 | dual specificity phosphatase 23 | 158716 | 132693 | 181036 | 35, 1, 16 |
| DUSP27 | dual specificity phosphatase 27 (putative) | 198842 | 143167 | 143190 | 16, 40, 18 |
| DYRK3 | dual specificity tyrosine phosphorylation regulated kinase 3 | 143479 | 143486 | 162889 | 38, 3, 6 |
| DNM3 | dynamin 3 | 197959 | 010165 | 135845 | 8, 2, 26 |
| DNAH14 | dynein axonemal heavy chain 14 | 185842 | 143786 | 143815 | 10, 4, 26 |
| DNALI1 | dynein axonemal light intermediate chain 1 | 163879 | 163877 | 134697 | 20, 17, 33 |
| E2F2 | E2F transcription factor 2 | 007968 | 088280 | 117318 | 21, 39, 22 |
| ELF3 | E74 like ETS transcription factor 3 | 163435 | 176393 | 170075 | 4, 42, 16 |
| EBNA1BP2 | EBNA1 binding protein 2 | 117395 | 186973 | 243710 | 17, 10, 19 |
| EVI5 | ecotropic viral integration site 5 | 067208 | 162676 | 122406 | 4, 31, 3 |
| EDARADD | EDAR associated death domain | 186197 | 086619 | 116977 | 24, 43, 9 |
| EFCAB14 | EF-hand calcium binding domain 14 | 159658 | 186118 | 142973 | 40, 29, 42 |
| EFCAB2 | EF-hand calcium binding domain 2 | 203666 | 153187 | 162849 | 4, 13, 34 |
| EFCAB7 | EF-hand calcium binding domain 7 | 203965 | 142856 | 079739 | 26, 7, 31 |
| EFHD2 | EF-hand domain family member D2 | 142634 | 142621 | 162438 | 34, 33, 37 |
| EGLN1 | egl-9 family hypoxia inducible factor 1 | 135766 | 010072 | 116918 | 34, 22, 7 |
| ELAVL4 | ELAV like RNA binding protein 4 | 162374 | 162373 | 142700 | |
| ELK4 | ELK4, ETS transcription factor | 158711 | 174514 | 158715 | 22, 17, 33 |
| ELOVL1 | ELOVL fatty acid elongase 1 | 066322 | 117399 | 159479 | 35, 37, 13 |
| ENAH | enabled homolog (Drosophila) | 154380 | 143815 | 143742 | 18, 4, 45 |
| ERO1B | endoplasmic reticulum oxidoreductase 1 beta | 086619 | 077585 | 186197 | 28, 38, 2 |
| ENSA | endosulfine alpha | 143420 | 143384 | 143457 | 3, 37, 6 |
| EDN2 | endothelin 2 | 127129 | 204060 | 127124 | 15, 23, 17 |
| ECE1 | endothelin converting enzyme 1 | 117298 | 075151 | 142794 | 43, 24, 42 |
| ENO1 | enolase 1 | 074800 | 142599 | 131686 | 9, 13, 27 |
| ECHDC2 | enoyl-CoA hydratase domain containing 2 | 121310 | 203995 | 116171 | 40, 26, 19 |
| EPHA10 | EPH receptor A10 | 183317 | 134690 | 185090 | 38, 16, 28 |
| EPHA2 | EPH receptor A2 | 142627 | 185519 | 142632 | 28, 27, 30 |
| EPHA8 | EPH receptor A8 | 070886 | 184677 | 173372 | 14, 36, 28 |
| EPHB2 | EPH receptor B2 | 133216 | 173369 | 215906 | 43, 16, 1 |
| EFNA1 | ephrin A1 | 169242 | 143590 | 169241 | 9, 34, 32 |
| EFNA3 | ephrin A3 | 143590 | 251246 | 169242 | 2, 6, 13 |
| EFNA4 | ephrin A4 | 243364 | 143537 | 251246 | 3, 43, 33 |
| EPS15 | epidermal growth factor receptor pathway substrate 15 | 085832 | 085831 | 117859 | 31, 26, 37 |
| EPHX1 | epoxide hydrolase 1 | 143819 | 143742 | 196187 | 2, 34, 1 |
| EPHX4 | epoxide hydrolase 4 | 172031 | 137948 | 230667 | 14, 27, 16 |
| EPS8L3 | EPS8 like 3 | 198758 | 134202 | 184371 | 37, 3, 16 |
| EDEM3 | ER degradation enhancing alpha-mannosidase like protein 3 | 116406 | 116667 | 135842 | 42, 18, 15 |
| EMC1 | ER membrane protein complex subunit 1 | 127463 | 127481 | 053372 | 7, 13, 38 |
| ERRFI1 | ERBB receptor feedback inhibitor 1 | 116285 | 116288 | 162426 | 40, 19, 10 |
| ERI3 | ERI1 exoribonuclease family member 3 | 117419 | 178028 | 187147 | 29, 34, 37 |
| ERMAP | erythroblast membrane associated protein (Scianna blood group) | 164010 | 177868 | 164011 | 42, 2, 26 |
| EPB41 | erythrocyte membrane protein band 4.1 | 159023 | 116329 | 253304 | 22, 19, 18 |
| ESPN | espin | 187017 | 069812 | 215788 | |
| ESRRG | estrogen related receptor gamma | 196482 | 042781 | 092978 | 32, 14, 34 |
| ETNK2 | ethanolamine kinase 2 | 143845 | 143842 | 143839 | 38, 17, 19 |
| ETV3 | ETS variant 3 | 117036 | 253831 | 143297 | 7, 16, 2 |
| ETV3L | ETS variant 3 like | 253831 | 132694 | 117036 | 35, 8, |
| EIF2B3 | eukaryotic translation initiation factor 2 B subunit gamma | 070785 | 117425 | 126107 | 18, 5, 16 |
| EIF2D | eukaryotic translation initiation factor 2D | 143486 | 266094 | 143479 | 9, 5, 45 |
| EIF3I | eukaryotic translation initiation factor 3 subunit I | 084623 | 160055 | 183615 | 7, 4, 34 |
| EIF4G3 | eukaryotic translation initiation factor 4 gamma 3 | 075151 | 127483 | 117298 | 40, 26, 11 |
| EVA1B | eva-1 homolog B | 142694 | 214193 | 196182 | 27, 26, 31 |
| EXOC8 | exocyst complex component 8 | 116903 | 116906 | 010072 | 1, 29, 22 |
| EXO1 | exonuclease 1 | 174371 | 162843 | 196289 | 6, 11, 39 |
| EXO5 | exonuclease 5 | 164002 | 187815 | 117010 | 6, 15, 44 |
| EXOSC10 | exosome component 10 | 171824 | 116649 | 198793 | 36, 45, 3 |
| EXTL1 | exostosin like glycosyltransferase 1 | 158008 | 158006 | 158014 | 8, 35, 25 |
| EXTL2 | exostosin like glycosyltransferase 2 | 162694 | 162692 | 162695 | 29, 6, 45 |
| ECM1 | extracellular matrix protein 1 | 143369 | 143374 | 143382 | 13, 3, 40 |
| EYA3 | EYA transcriptional coactivator and phosphatase 3 | 158161 | 158156 | 169403 | 22, 39, 43 |
| F11R | F11 receptor | 158769 | 158764 | 270149 | 24, 30, 42 |
| FAM20B | FAM20B, glycosaminoglycan xylosylkinase | 116199 | 116194 | 186283 | 1, 22, 34 |
| FAM102B | family with sequence similarity 102 member B | 162636 | 186086 | 162639 | 26, 35, 9 |
| FAM110D | family with sequence similarity 110 member D | 197245 | 175087 | 282872 | 1, 20, 32 |
| FAM129A | family with sequence similarity 129 member A | 135842 | 116406 | 121481 | 42, 22, 33 |
| FAM131C | family with sequence similarity 131 member C | 185519 | 184908 | 142627 | 8, 28, 6 |
| FAM132A | family with sequence similarity 132 member A | 184163 | 176022 | 160087 | 2, 17, 36 |
| FAM151A | family with sequence similarity 151 member A | 162391 | 162390 | 184313 | 17, 40, 36 |
| FAM159A | family with sequence similarity 159 member A | 182183 | 116157 | 162377 | 22, 10, 9 |
| FAM163A | family with sequence similarity 163 member A | 143340 | 162782 | 169905 | 26, 39, 38 |
| FAM167B | family with sequence similarity 167 member B | 183615 | 084623 | 182866 | 13, 29, 35 |
| FAM177B | family with sequence similarity 177 member B | 197520 | 162819 | 154309 | 42, 30, 5 |
| FAM183A | family with sequence similarity 183 member A | 186973 | 117394 | 117395 | 31, 20, 33 |
| FAM189B | family with sequence similarity 189 member B | 160767 | 177628 | 116521 | 21, 26, 25 |
| FAM19A3 | family with sequence similarity 19 member A3, C-C motif chemokine like | 184599 | 155367 | 155380 | 14, 6, 15 |
| FAM212B | family with sequence similarity 212 member B | 197852 | 116473 | 064703 | |
| FAM213B | family with sequence similarity 213 member B | 157870 | 157873 | 142606 | 3, 36, 2 |
| FAM229A | family with sequence similarity 229 member A | 225828 | 162526 | 160058 | 24, 39, 3 |
| FAM231A | family with sequence similarity 231 member A | 237847 | 219481 | 279132 | |
| FAM231B | family with sequence similarity 231 member B | 268991 | 157191 | 219481 | |
| FAM231C | family with sequence similarity 231 member C | 279132 | 237847 | 058453 | |
| FAM231D | family with sequence similarity 231 member D | 272058 | 263464 | 150337 | |
| FAM43B | family with sequence similarity 43 member B | 183114 | 090432 | 158825 | 2, 6, 44 |
| FAM46B | family with sequence similarity 46 member B | 158246 | 253368 | 280403 | 35, 14, 33 |
| FAM46C | family with sequence similarity 46 member C | 183508 | 198162 | 196505 | 42, 31, 4 |
| FAM63A | family with sequence similarity 63 member A | 143409 | 143412 | 143363 | 24, 40, 42 |
| FAM69A | family with sequence similarity 69 member A | 154511 | 122406 | 143033 | 3, 30, 17 |
| FAM71A | family with sequence similarity 71 member A | 162771 | 162772 | 123685 | 38, 8, |
| FAM72A | family with sequence similarity 72 member A | 196550 | 198049 | 266028 | 17, 16, |
| FAM72B | family with sequence similarity 72 member B | 188610 | 198019 | 171943 | 22, 21, 18 |
| FAM72C | family with sequence similarity 72 member C | 263513 | 203818 | 271567 | |
| FAM72D | family with sequence similarity 72 member D | 215784 | 196369 | 281571 | 24, 4, 5 |
| FAM76A | family with sequence similarity 76 member A | 009780 | 126709 | 117758 | 13, 29, 22 |
| FAM78B | family with sequence similarity 78 member B | 188859 | 143179 | 143157 | 14, 26, 4 |
| FAM89A | family with sequence similarity 89 member A | 182118 | 173409 | 119283 | 26, 32, 35 |
| FAAP20 | Fanconi anemia cere complex associated protein 20 | 162585 | 067606 | 157933 | 22, 41, 23 |
| FUBP1 | far upstream element binding protein 1 | 162613 | 162614 | 162616 | 7, 1, 12 |
| FDPS | farnesyl diphosphate synthase | 160752 | 143627 | 160753 | 21, 19, 27 |
| FAF1 | Fas associated factor 1 | 185104 | 142700 | 123080 | 10, 41, 25 |
| FASLG | Fas ligand | 117560 | 094975 | 120337 | 5, 7, 36 |
| FAAH | fatty acid amide hydrolase | 117480 | 117481 | 197587 | 38, 24, 27 |
| FABP3 | fatty acid binding protein 3 | 121769 | 121766 | 168528 | 15, 25, 20 |
| FBXO2 | F-box protein 2 | 116661 | 204624 | 132879 | 29, 26, 20 |
| FBXO28 | F-box protein 28 | 143756 | 143514 | 143753 | 29, 4, 39 |
| FBXO42 | F-box protein 42 | 037637 | 132881 | 055070 | 11, 4, 39 |
| FBXO44 | F-box protein 44 | 132879 | 116661 | 116663 | 41, 8, 36 |
| FBXO6 | F-box protein 6 | 116663 | 132879 | 116670 | 6, 35, 23 |
| FCAMR | Fc fragment of IgA and IgM receptor | 162897 | 162896 | 182795 | 17, 30, 16 |
| FCER1A | Fc fragment of IgE receptor Ia | 179639 | 213088 | 196266 | 33, 6, 30 |
| FCER1G | Fc fragment of IgE receptor Ig | 158869 | 158864 | 158874 | 5, 2, 40 |
| FCGR1A | Fc fragment of IgG receptor Ia | 150337 | 272058 | 203814 | 26, 30, 32 |
| FCGR1B | Fc fragment of IgG receptor Ib | 198019 | 281741 | 188610 | 34, 32, 30 |
| FCGR2A | Fc fragment of IgG receptor IIa | 143226 | 188931 | 173110 | |
| FCGR2B | Fc fragment of IgG receptor IIb | 072694 | 162747 | 132185 | 32, 1, 22 |
| FCGR2C | Fc fragment of IgG receptor IIc (gene/pseudogene) | 244682 | 203747 | 162747 | 13, 32, 10 |
| FCGR3A | Fc fragment of IgG receptor IIIa | 203747 | 173110 | 244682 | 5, 36, 10 |
| FCGR3B | Fc fragment of IgG receptor IIIb | 162747 | 244682 | 072694 | |
| FCMR | Fc fragment of IgM receptor | 162894 | 162892 | 162896 | |
| FCRL1 | Fc receptor like 1 | 163534 | 132704 | 073754 | 36, 22, 30 |
| FCRL2 | Fc receptor like 2 | 132704 | 160856 | 163534 | 22, 24, 21 |
| FCRL3 | Fc receptor like 3 | 160856 | 163518 | 132704 | 22, 36, 21 |
| FCRL4 | Fc receptor like 4 | 163518 | 143297 | 160856 | 22, |
| FCRL5 | Fc receptor like 5 | 143297 | 117036 | 163518 | 22, 24, 36 |
| FCRL6 | Fc receptor like 6 | 181036 | 158716 | 158714 | 36, 23, 35 |
| FCRLA | Fc receptor like A | 132185 | 072694 | 162746 | 21, 36, 35 |
| FCRLB | Fc receptor like B | 162746 | 132185 | 081721 | 10, 35, 23 |
| FLVCR1 | feline leukemia virus subgroup C cellular receptor 1 | 162769 | 185523 | 143494 | 1, 31, 23 |
| FRRS1 | ferric chelate reductase 1 | 156869 | 099260 | 162688 | 4, 24, 32 |
| FGGY | FGGY carbohydrate kinase domain containing | 172456 | 177606 | 134709 | 10, 26, 1 |
| FGR | FGR proto-oncogene, Src family tyrosine kinase | 000938 | 126705 | 126709 | 36, 5, 21 |
| FMOD | fibromodulin | 122176 | 159388 | 188783 | 10, 6, 43 |
| FNDC10 | fibronectin type III domain containing 10 | 228594 | 279244 | 197530 | 3, 26, 14 |
| FNDC5 | fibronectin type III domain containing 5 | 160097 | 116497 | 121905 | |
| FNDC7 | fibronectin type III domain containing 7 | 143107 | 134186 | 116266 | 38, 23, 32 |
| FCN3 | ficolin 3 | 142748 | 142733 | 174950 | 20, 19, 17 |
| FLG | filaggrin | 143631 | 197915 | 143520 | 35, 23, 24 |
| FLG2 | filaggrin family member 2 | 143520 | 143631 | 143536 | 35, |
| FBLIM1 | filamin binding LIM protein 1 | 162458 | 162460 | 233954 | 18, 37, 34 |
| FLAD1 | flavin adenine dinucleotide synthetase 1 | 160688 | 173207 | 163352 | 13, 43, 9 |
| FMO1 | flavin containing monooxygenase 1 | 010932 | 094963 | 076258 | 17, 15, 23 |
| FMO2 | flavin containing monooxygenase 2 | 094963 | 007933 | 010932 | 42, 30, 45 |
| FMO3 | flavin containing monooxygenase 3 | 007933 | 117501 | 094963 | 19, 40, 13 |
| FMO4 | flavin containing monooxygenase 4 | 076258 | 010932 | 117523 | 26, 29, 18 |
| FMO5 | flavin containing monooxygenase 5 | 131781 | 131791 | 131778 | 42, 29, 19 |
| FHAD1 | forkhead associated phosphopeptide binding domain 1 | 142621 | 171729 | 142634 | |
| FOXD2 | forkhead box D2 | 186564 | 186790 | 269113 | 5, 27, 16 |
| FOXD3 | forkhead box D3 | 187140 | 125703 | 088035 | 11, 15, 17 |
| FOXE3 | forkhead box E3 | 186790 | 162368 | 186564 | 14, 33, 38 |
| FOXJ3 | forkhead box J3 | 198815 | 197273 | 177181 | 37, 1, 9 |
| FOXO6 | forkhead box O6 | 204060 | 010803 | 127129 | |
| FMN2 | formin 2 | 155816 | 133019 | 180875 | 29, 11, 10 |
| FNBP1L | formin binding protein 1 like | 137942 | 117505 | 137936 | 43, 11, 42 |
| FHL3 | four and a half LIM domains 3 | 183386 | 183431 | 183520 | 25, 9, 22 |
| FPGT-TNNI3K | FPGT-TNNI3K readthrough | 259030 | 254685 | 116783 | |
| FPGT | fucose-1-phosphate guanylyltransferase | 254685 | 162620 | 259030 | |
| FUCA1 | fucosidase, alpha-L-1, tissue | 179163 | 117305 | 188822 | 5, 36, 29 |
| FH | fumarate hydratase | 091483 | 182901 | 117009 | 29, 35, 21 |
| GNL2 | G protein nucleolar 2 | 134697 | 163879 | 169218 | 45, 1, 13 |
| GNAI3 | G protein subunit alpha i3 | 065135 | 156097 | 134183 | |
| GNAT2 | G protein subunit alpha transducin 2 | 134183 | 065135 | 116337 | 14, 38, 33 |
| GNB1 | G protein subunit beta 1 | 078369 | 008130 | 169885 | 14, 40, 20 |
| GNG12 | G protein subunit gamma 12 | 172380 | 116717 | 162595 | 45, 4, 35 |
| GNG4 | G protein subunit gamma 4 | 168243 | 162885 | 143669 | 28, 26, 8 |
| GNG5 | G protein subunit gamma 5 | 174021 | 117133 | 117151 | 11, 1, 43 |
| GPR137B | G protein-coupled receptor 137B | 077585 | 116962 | 086619 | 35, 17, 29 |
| GPR153 | G protein-coupled receptor 153 | 158292 | 173673 | 097021 | 18, 1, 34 |
| GPR157 | G protein-coupled receptor 157 | 180758 | 142583 | 049239 | 24, 42, 40 |
| GPR161 | G protein-coupled receptor 161 | 143147 | 143164 | 143155 | 34, 44, 24 |
| GPR25 | G protein-coupled receptor 25 | 170128 | 118200 | 163362 | 37, |
| GPR3 | G protein-coupled receptor 3 | 181773 | 174950 | 158195 | 6, 14, 37 |
| GPR37L1 | G protein-coupled receptor 37 like 1 | 170075 | 163435 | 143862 | 8, 17, 38 |
| GPR52 | G protein-coupled receptor 52 | 203737 | 152061 | 116161 | 23, 17, |
| GPR61 | G protein-coupled receptor 61 | 156097 | 181754 | 065135 | |
| GPR88 | G protein-coupled receptor 88 | 181656 | 079335 | 162692 | 11, 36, 10 |
| GPR89A | G protein-coupled receptor 89A | 117262 | 162825 | 174827 | |
| GPR89B | G protein-coupled receptor 89B | 188092 | 121634 | 263956 | 13, 38, 42 |
| G0S2 | G0/G1 switch 2 | 123689 | 196878 | 117594 | 1, 26, 3 |
| GABPB2 | GA binding protein transcription factor beta subunit 2 | 143458 | 213190 | 143434 | 1, 2, 3 |
| LGALS8 | galectin 8 | 116977 | 186197 | 119285 | 42, 36, 24 |
| GABRD | gamma-aminobutyric acid type A receptor delta subunit | 187730 | 142609 | 067606 | 8, 26, 15 |
| GDAP2 | ganglioside induced differentiation associated protein 2 | 196505 | 183508 | 065183 | 9, 24, 30 |
| GJA4 | gap junction protein alpha 4 | 187513 | 188910 | 116544 | 32, 1, 23 |
| GJA5 | gap junction protein alpha 5 | 265107 | 162836 | 121634 | 32, 20, 37 |
| GJA8 | gap junction protein alpha 8 | 121634 | 265107 | 188092 | 38, 44, 14 |
| GJA9 | gap junction protein alpha 9 | 131233 | 274944 | 158315 | |
| GJB3 | gap junction protein beta 3 | 188910 | 189433 | 187513 | 9, 13, 37 |
| GJB4 | gap junction protein beta 4 | 189433 | 189280 | 188910 | 42, 24, 35 |
| GJB5 | gap junction protein beta 5 | 189280 | 163866 | 189433 | 30, 13, 35 |
| GJC2 | gap junction protein gamma 2 | 198835 | 143774 | 181873 | 9, 27, 14 |
| GATAD2B | GATA zinc finger domain containing 2B | 143614 | 143554 | 198837 | 18, 24, 22 |
| GPBP1L1 | GC-rich promoter binding protein 1 like 1 | 159592 | 159588 | 159596 | 42, 39, 38 |
| GTF2B | general transcription factor IIB | 137947 | 065243 | 137944 | 29, 4, 30 |
| GGPS1 | geranylgeranyl diphosphate synthase 1 | 152904 | 054267 | 116957 | 4, 31, 29 |
| GCSAML | germinal center associated signaling and motility like | 169224 | 177535 | 196242 | |
| GIPC2 | GIPC PDZ domain containing family member 2 | 137960 | 162616 | 122420 | 4, 17, 36 |
| GLIS1 | GLIS family zinc finger 1 | 174332 | 143006 | 058804 | 17, 14, 12 |
| GLMN | glomulin, FKBP associated protein | 174842 | 203910 | 122484 | 9, 26, 10 |
| GMEB1 | glucocorticoid modulatory element binding protein 1 | 162419 | 188060 | 198492 | 22, 24, 16 |
| GBA | glucosylceramidase beta | 177628 | 173171 | 160767 | 32, |
| GRIK3 | glutamate ionotropic receptor kainate type subunit 3 | 163873 | 119535 | 163874 | 14, 8, 20 |
| ERICH3 | glutamate rich 3 | 178965 | 162621 | 116791 | 31, 38, 20 |
| GLUL | glutamate-ammonia ligase | 135821 | 179930 | 203730 | 30, 26, 14 |
| GCLM | glutamate-cysteine ligase modifier subunit | 023909 | 067334 | 198691 | 4, 29, 32 |
| EPRS | glutamyl-prolyl-tRNA synthetase | 136628 | 196660 | 162813 | 2, 3, 43 |
| GLRX2 | glutaredoxin 2 | 023572 | 116747 | 134371 | 10, 2, 38 |
| GPX7 | glutathione peroxidase 7 | 116157 | 134744 | 182183 | 22, 15, 10 |
| GSTM1 | glutathione S-transferase mu 1 | 134184 | 213366 | 134201 | 26, 13, 23 |
| GSTM2 | glutathione S-transferase mu 2 | 213366 | 168765 | 134184 | 2, 26, 40 |
| GSTM3 | glutathione S-transferase mu 3 | 134202 | 134201 | 198758 | 5, 3, 1 |
| GSTM4 | glutathione S-transferase mu 4 | 168765 | 116337 | 213366 | 34, 31, 37 |
| GSTM5 | glutathione S-transferase mu 5 | 134201 | 134184 | 134202 | 18, 26, 15 |
| GNPAT | glyceronephosphate O-acyltransferase | 116906 | 143633 | 116903 | 41, 38, 36 |
| GPA33 | glycoprotein A33 | 143167 | 143194 | 198842 | 16, 3, 39 |
| GLMP | glycosylated lysosomal membrane protein | 198715 | 163472 | 189030 | 32, 6, 37 |
| GOLPH3L | golgi phosphoprotein 3 like | 143457 | 143420 | 143452 | 42, 11, 6 |
| GOLT1A | golgi transport 1A | 174567 | 170498 | 143850 | 29, 28, 19 |
| GORAB | golgin, RAB6 interacting | 120370 | 203740 | 116132 | 7, 45, 42 |
| GON4L | gon-4 like | 116580 | 132676 | 132718 | 40, 31, 37 |
| GPATCH2 | G-patch domain containing 2 | 092978 | 196482 | 162814 | 39, 10, 42 |
| GPATCH3 | G-patch domain containing 3 | 198746 | 142751 | 090273 | 39, 36, 3 |
| GPATCH4 | G-patch domain containing 4 | 160818 | 163382 | 280316 | 21, 27, 7 |
| GPN2 | GPN-loop GTPase 2 | 142751 | 175793 | 198746 | 35, 1, 6 |
| GPSM2 | G-protein signaling modulator 2 | 121957 | 274068 | 121940 | 31, 18, 10 |
| GRHL3 | grainyhead like transcription factor 3 | 158055 | 185436 | 001460 | 24, 29, 18 |
| GREM2 | gremlin 2, DAN family BMP antagonist | 180875 | 155816 | 182901 | 10, 15, 2 |
| GADD45A | growth arrest and DNA damage inducible alpha | 116717 | 142864 | 172380 | 29, 17, 6 |
| GFI1 | growth factor independent 1 transcriptional repressor | 162676 | 122484 | 067208 | 39, 7, 5 |
| GBP1 | guanylate binding protein 1 | 117228 | 117226 | 162645 | 36, 43, 44 |
| GBP2 | guanylate binding protein 2 | 162645 | 117228 | 213512 | 36, 2, 5 |
| GBP3 | guanylate binding protein 3 | 117226 | 213516 | 117228 | 45, 36, 13 |
| GBP4 | guanylate binding protein 4 | 162654 | 213512 | 154451 | 44, 1, 4 |
| GBP5 | guanylate binding protein 5 | 154451 | 162654 | 183347 | 5, 36, 13 |
| GBP7 | guanylate binding protein 7 | 213512 | 162645 | 162654 | 19, 37, 14 |
| GBP6 | guanylate binding protein family member 6 | 183347 | 154451 | 197147 | 24, 13, 42 |
| GUCA2A | guanylate cyclase activator 2A | 197273 | 044012 | 198815 | 16, 25, 10 |
| GUCA2B | guanylate cyclase activator 2B | 044012 | 127124 | 197273 | 16, 38, |
| GUK1 | guanylate kinase 1 | 143774 | 162910 | 198835 | 34, 3, 41 |
| HLX | H2.0 like homeobox | 136630 | 186205 | 143507 | 9, 13, 5 |
| H3F3A | H3 histone family member 3A | 163041 | 143751 | 182827 | |
| HAX1 | HCLS1 associated protein X-1 | 143575 | 143569 | 143595 | 34, 31, 21 |
| HEATR1 | HEAT repeat containing 1 | 119285 | 116977 | 077522 | 18, 3, 30 |
| HSPA6 | heat shock protein family A (Hsp70) member 6 | 173110 | 143226 | 203747 | 26, 42, 32 |
| HSPB11 | heat shock protein family B (small) member 11 | 081870 | 211452 | 116212 | |
| HSPB7 | heat shock protein family B (small) member 7 | 173641 | 183888 | 186510 | 15, 25, 1 |
| HECTD3 | HECT domain E3 ubiquitin protein ligase 3 | 126107 | 070785 | 126088 | 42, 23, 6 |
| HHAT | hedgehog acyltransferase | 054392 | 082497 | 143473 | 42, 11, 15 |
| HMCN1 | hemicentin 1 | 143341 | 116679 | 116690 | 18, 24, 11 |
| HFE2 | hemochromatosis type 2 (juvenile) | 168509 | 265972 | 271425 | 25, 19, 15 |
| HENMT1 | HEN1 methyltransferase homolog 1 | 162639 | 162636 | 134186 | 38, 9, 30 |
| HS2ST1 | heparan sulfate 2-O-sulfotransferase 1 | 153936 | 183291 | 267561 | 11, 22, 3 |
| HSPG2 | heparan sulfate proteoglycan 2 | 142798 | 187942 | 219073 | 26, 1, 12 |
| HDGF | hepatoma-derived growth factor | 143321 | 143314 | 143294 | 9, 34, 4 |
| HHLA3 | HERV-H LTR-associating 3 | 197568 | 118454 | 279230 | 34, 27, 7 |
| HES2 | hes family bHLH transcription factor 2 | 069812 | 097021 | 187017 | |
| HES3 | hes family bHLH transcription factor 3 | 173673 | 116237 | 158292 | 12, |
| HES4 | hes family bHLH transcription factor 4 | 188290 | 187642 | 187608 | 26, 37, 6 |
| HES5 | hes family bHLH transcription factor 5 | 197921 | 157881 | 157873 | 15, 14, 8 |
| HEYL | hes related family bHLH transcription factor with YRPW motif-like | 163909 | 090621 | 116981 | 25, 15, 32 |
| HP1BP3 | heterochromatin protein 1 binding protein 3 | 127483 | 189410 | 075151 | 39, 42, 11 |
| HNRNPCL1 | heterogeneous nuclear ribonucleoprotein C-like 1 | 179172 | 239810 | 120952 | 44, |
| HNRNPCL2 | heterogeneous nuclear ribonucleoprotein C-like 2 | 275774 | 277058 | 280267 | |
| HNRNPCL3 | heterogeneous nuclear ribonucleoprotein C-like 3 | 277058 | 229571 | 275774 | |
| HNRNPCL4 | heterogeneous nuclear ribonucleoprotein C-like 4 | 179412 | 280267 | 204505 | |
| HNRNPR | heterogeneous nuclear ribonucleoprotein R | 125944 | 179546 | 125945 | |
| HNRNPU | heterogeneous nuclear ribonucleoprotein U | 153187 | 203667 | 203666 | 29, 22, 40 |
| H6PD | hexose-6-phosphate dehydrogenase/glucose 1-dehydrogenase | 049239 | 180758 | 171621 | 22, 34, 24 |
| HFM1 | HFM1, ATP dependent DNA helicase homolog | 162669 | 122482 | 097046 | 38, 44, 25 |
| HHIPL2 | HHIP like2 | 143512 | 143507 | 143498 | 40, 38, 28 |
| HMGB4 | high mobility group box 4 | 176256 | 121904 | 142698 | |
| HMGN2 | high mobility group nudeosomal binding domain 2 | 198830 | 117682 | 117676 | 41, 21, 30 |
| HPCA | hippocalcin | 121905 | 160097 | 121900 | 14, 8, 28 |
| HPCAL4 | hippocalcin like 4 | 116983 | 116981 | 084072 | 8, 2, 14 |
| HIST2H2AA3 | histone cluster 2 H2 A family member a3 | 203812 | 203811 | 272196 | 33, 7, 18 |
| HIST2H2AA4 | histone cluster 2 H2 A family member a4 | 272196 | 203812 | 203852 | |
| HIST2H2AB | histone cluster 2 H2 A family member b | 184270 | 184260 | 178096 | 33, |
| HIST2H2AC | histone cluster 2 H2 A family member c | 184260 | 184678 | 184270 | 30, 7, 15 |
| HIST2H2BE | histone cluster 2 H2B family member e | 184678 | 270276 | 184260 | 39, 31, 13 |
| HIST2H2BF | histone cluster 2 H2B family member f | 203814 | 150337 | 183598 | 36, 13, 42 |
| HIST2H3A | histone cluster 2 H3 family member a | 203852 | 272196 | 270276 | |
| HIST2H3C | histone cluster 2 H3 family member c | 203811 | 270882 | 203812 | 28, 44, |
| HIST2H3D | histone cluster 2 H3 family member d | 183598 | 203814 | 270882 | 7, |
| HIST2H3PS2 | Histone cluster 2 H3 pseudogene 2 | 203818 | 171943 | 263513 | |
| HIST2H4A | histone cluster 2 H4 family member a | 270882 | 183598 | 203811 | 24, 37, 5 |
| HIST2H4B | histone cluster 2 H4 family member b | 270276 | 203852 | 184678 | |
| HIST3H2A | histone cluster 3 H2A | 181218 | 168148 | 196890 | 2, 3, 34 |
| HIST3H2BB | histone cluster 3 H2B family member b | 196890 | 181218 | 168159 | 3, 30, 24 |
| HIST3H3 | histone cluster 3 H3 | 168148 | 162931 | 181218 | 10, 38, |
| HDAC1 | histone deacetylase 1 | 116478 | 182866 | 175130 | 37, 24, 23 |
| HIPK1 | homeodomain interacting protein kinase 1 | 163349 | 118655 | 116774 | |
| HOOK1 | hook microtubule tethering protein 1 | 134709 | 172456 | 134716 | 29, 31, 38 |
| HORMAD1 | HORMA domain containing 1 | 143452 | 143457 | 163131 | 38, 23, 42 |
| HRNR | homerin | 197915 | 215853 | 143631 | 27, 38, |
| HIVEP3 | human immunodeficiency virus type I enhancer binding protein 3 | 127124 | 127129 | 044012 | 21, 40, 22 |
| HAPLN2 | hyaluronan and proteoglycan link protein 2 | 132702 | 280316 | 132692 | 26, 8, 17 |
| HAO2 | hydroxyacid oxidase 2 | 116882 | 116874 | 203859 | 18, 19, 17 |
| HSD3B1 | hydroxy-delta-5-steroid dehydrogenase, 3 beta- and steroid delta-isomerase 1 | 203857 | 203859 | 143067 | 32, 16, 35 |
| HSD3B2 | hydroxy-delta-5-steroid dehydrogenase, 3 beta- and steroid delta-isomerase 2 | 203859 | 116882 | 203857 | 2, 27, 16 |
| HYI | hydroxypyruvate isomerase (putative) | 178922 | 198198 | 142949 | 37, 34, 27 |
| HSD11B1 | hydroxysteroid 11-beta dehydrogenase 1 | 117594 | 123689 | 009790 | 27, 19, 44 |
| HSD17B7 | hydroxysteroid 17-beta dehydrogenase 7 | 132196 | 162733 | 185860 | 39, 1, 24 |
| HCN3 | hyperpolarization activated cyclic nucleotide gated potassium channel 3 | 143630 | 176444 | 143627 | 1, 31, 17 |
| HCRTR1 | hypocretin receptor 1 | 121764 | 142910 | 162517 | 28, 8, |
| IBA57 | IBA57 homolog, iron-sulfur cluster assembly | 181873 | 198835 | 162913 | 40, 26, 31 |
| IER5 | immediate early response 5 | 162783 | 153029 | 198216 | 32, 37, 7 |
| IGSF21 | immunoglobin superfamily member 21 | 117154 | 117148 | 179023 | 45, 8, 14 |
| ILDR2 | immunoglobulin like domain containing receptor 2 | 143195 | 152382 | 143194 | |
| IGSF3 | immunoglobulin superfamily member 3 | 143061 | 116815 | 116824 | 24, 30, 35 |
| IGSF8 | immunoglobulin superfamily member 8 | 162729 | 162728 | 018625 | 35, 26, 8 |
| IGSF9 | immunoglobulin superfamily member 9 | 085552 | 158710 | 162723 | 18, 4, 27 |
| IGFN1 | immunoglobulin-like and fibronectin type III domain containing 1 | 163395 | 116857 | 081277 | 40, 18, 4 |
| IPO13 | importin 13 | 117408 | 117407 | 132768 | 38, 8, 25 |
| IPO9 | importin 9 | 198700 | 134369 | 198892 | 18, 9, 26 |
| IVNS1ABP | influenza virus NS1A binding protein | 116679 | 116668 | 143341 | 24, 42, 1 |
| ID3 | inhibitor of DNA binding 3, HLH protein | 117318 | 007968 | 197880 | 11, 35, 26 |
| IKBKE | inhibitor of kappa light polypeptide gene enhancer in B-cells, kinase epsilon | 263528 | 266028 | 266094 | 43, 39, 36 |
| INPP5B | inositol polyphosphate-5-phosphatase B | 204084 | 188786 | 183431 | 22, 21, 42 |
| ITPKB | inositol-trisphosphate 3-kinase B | 143772 | 203685 | 143801 | 22, 26, 41 |
| INSL5 | insulin like 5 | 172410 | 152760 | 152763 | |
| INSRR | insulin receptor related receptor | 027644 | 198400 | 187800 | 38, 17, |
| INTS3 | integrator complex subunit 3 | 143624 | 169418 | 279767 | 18, 43, 21 |
| INTS7 | integrator complex subunit 7 | 143493 | 123684 | 143476 | 24, 38, 39 |
| ITGA10 | integrin subunit alpha 10 | 143127 | 198483 | 131779 | 42, 11, 25 |
| ITGB3BP | integrin subunit beta 3 binding protein | 142856 | 088035 | 203965 | 4, 34, 10 |
| ITLN1 | intelectin 1 | 179914 | 122223 | 158764 | 1, 15, 16 |
| ITLN2 | intelectin 2 | 158764 | 179914 | 158769 | 16, 10, 25 |
| IFI6 | interferon alpha inducible protein 6 | 126709 | 000938 | 009780 | |
| IFI16 | interferon gamma inducible protein 16 | 163565 | 163564 | 163568 | 39, 22, 18 |
| IFI44 | interferon induced protein 44 | 137965 | 137959 | 162618 | 13, 31, 6 |
| IFI44L | interferon induced protein 44 like | 137959 | 122420 | 137965 | 4, 36, 44 |
| IFNLR1 | interferon lambda receptor 1 | 185436 | 142677 | 158055 | 21, 22, 33 |
| IRF2BP2 | interferon regulatory factor 2 binding protein 2 | 168264 | 059588 | 279261 | 1, 11, 45 |
| IRF6 | interferon regulatory factor 6 | 117595 | 162757 | 117597 | 42, 13, 30 |
| ISG20L2 | interferon stimulated exonuclease gene 20 like 2 | 143319 | 143320 | 143303 | 9, 38, 1 |
| IL10 | interleukin 10 | 136634 | 162889 | 142224 | 36, 5, 10 |
| IL12RB2 | interleukin 12 receptor subunit beta 2 | 081985 | 162594 | 142864 | 42, 39, 35 |
| IL19 | interleukin 19 | 142224 | 136634 | 162891 | 44, 20, |
| IL20 | interleukin 20 | 162891 | 142224 | 162892 | 30, |
| IL22RA1 | interleukin 22 receptor subunit alpha 1 | 142677 | 142661 | 185436 | 4, 2, 13 |
| IL23R | interleukin 23 receptor | 162594 | 203963 | 081985 | 7, 30, 38 |
| IL24 | interleukin 24 | 162892 | 162891 | 162894 | 22, 45, 20 |
| IL6R | interleukin 6 receptor | 160712 | 143515 | 169291 | 18, 5, 36 |
| ILF2 | interleukin enhancer binding factor 2 | 143621 | 143553 | 169418 | 30, 11, 12 |
| IFFO2 | intermediate filament family orphan 2 | 169991 | 159423 | 127481 | 18, 35, 13 |
| IPP | intracistemal A particle-promoted polypeptide | 197429 | 159596 | 086015 | 40, 34, 6 |
| IVL | involucrin | 163207 | 163206 | 283227 | 18, 24, 4 |
| DIO1 | iodothyronine deiodinase 1 | 211452 | 058799 | 081870 | 17, 19, 40 |
| IQCC | IQ motif containing C | 160051 | 160050 | 222046 | 2, 13, 9 |
| IQGAP3 | IQ motif containing GTPase activating protein 3 | 183856 | 116604 | 187862 | 7, 16, 22 |
| ISG15 | ISG15 ubiquitin-like modifier | 187608 | 188290 | 188157 | 44, 23, 33 |
| IARS2 | isoleucyl-tRNA synthetase 2, mitochondrial | 067704 | 162813 | 118873 | 7, 22, 23 |
| ICMT | isoprenylcysteine carboxyl methyltransferase | 116237 | 158286 | 173673 | 6, 11, 35 |
| JAK1 | Janus kinase 1 | 162434 | 162437 | 162433 | 18, 22, 40 |
| JMJD4 | jumonji domain containing 4 | 081692 | 143740 | 185888 | |
| JTB | jumping translocation breakpoint | 143543 | 143578 | 143545 | 21, 7, 27 |
| JUN | Jun proto-oncogene, AP-1 transcription factor subunit | 177606 | 162601 | 172456 | 3, 1, 29 |
| KPNA6 | karyopherin subunit alpha 6 | 025800 | 121775 | 084652 | 18, 43, 42 |
| KAZN | kazrin, periplakin interacting protein | 189337 | 116731 | 171729 | 29, 14, 32 |
| KLHDC7A | kelch domain containing 7A | 179023 | 117154 | 009709 | 17, 42, 24 |
| KLHDC8A | kelch domain containing 8A | 162873 | 163545 | 186007 | 2, 27, 40 |
| KLHDC9 | kelch domain containing 9 | 162755 | 143217 | 143256 | 29, 38, 26 |
| KLHL12 | kelch like family member 12 | 117153 | 183155 | 159346 | 38, 1, 42 |
| KLHL17 | kelch like family member 17 | 187961 | 188976 | 187583 | 9, 3, 14 |
| KLHL20 | kelch like family member 20 | 076321 | 232113 | 120334 | 11, 13, 29 |
| KLHL21 | kelch like family member 21 | 162413 | 204859 | 116273 | 42, 35, 8 |
| KRTCAP2 | keratinocyte associated protein 2 | 163463 | 179085 | 273088 | 31, 19, 40 |
| KDF1 | keratinocyte differentiation factor 1 | 175707 | 131910 | 253368 | 3, 40, 37 |
| KPRP | keratinocyte proline rich protein | 203786 | 198854 | 240386 | 35, 10, |
| KHDRBS1 | KH RNA binding domain containing, signal transduction associated 1 | 121774 | 184007 | 121775 | 6, 34, 15 |
| KIAA0040 | KIAA0040 | 235750 | 120332 | 116147 | 42, 32, 39 |
| KIAA0319L | KIAA0319 like | 142687 | 146463 | 020129 | 42, 40, 9 |
| KIAA0907 | KIAA0907 | 132680 | 143622 | 173080 | 19, 31, 2 |
| KIAA1107 | KIAA1107 | 069712 | 189195 | 203910 | 4, 18, 8 |
| KIAA1324 | KIAA1324 | 116299 | 179902 | 031698 | 42, 31, 23 |
| KIAA1522 | KIAA1522 | 162522 | 162520 | 134684 | 26, 4, 9 |
| KIAA1614 | KIAA1614 | 135835 | 143324 | 135823 | 1, 14, 8 |
| KIAA2013 | KIAA2013 | 116685 | 120937 | 083444 | |
| KIRREL | kin of IRRE like (Drosophila) | 183853 | 073754 | 158473 | |
| KIFAP3 | kinesin associated protein 3 | 075945 | 000457 | 203740 | 8, 38, 10 |
| KIF14 | kinesin family member 14 | 118193 | 162702 | 118197 | 18, 23, 22 |
| KIF17 | kinesin family member 17 | 117245 | 244038 | 189410 | 38, 36, 32 |
| KIF1B | kinesin family member 1B | 054523 | 130939 | 142657 | 25, |
| KIF21B | kinesin family member 21B | 116852 | 163362 | 081248 | 14, 5, 22 |
| KIF26B | kinesin family member 26B | 162849 | 203666 | 185420 | 39, 27, 12 |
| KIF2C | kinesin family member 2C | 142945 | 198520 | 142937 | 38, 39, 21 |
| KNCN | kinocilin | 162456 | 282881 | 079277 | 38, 8, |
| KISS1 | KiSS-1 metastasis-suppressor | 170498 | 143839 | 174567 | 32, 35, 15 |
| KANK4 | KN motif and ankyrin repeat domains 4 | 132854 | 240563 | 162607 | 11, 25, 32 |
| KLF17 | Kruppel like factor 17 | 171872 | 196517 | 283039 | 38, 6, 8 |
| KLF18 | Kruppel like factor 18 | 283039 | 171872 | 178028 | |
| KTI12 | KTI12 chromatin associated homolog | 198841 | 117862 | 134717 | |
| KMO | kynurenine 3-monooxygenase | 117009 | 091483 | 054277 | 32, 19, 41 |
| KYAT3 | kynurenine aminotransferase 3 | 137944 | 137947 | 213516 | |
| LACTBL1 | lactamase beta like 1 | 215906 | 133216 | 227868 | |
| LAD1 | ladinin 1 | 159166 | 118194 | 159173 | 4, 18, 37 |
| LMNA | lamin A/C | 160789 | 254726 | 196189 | 24, 10, 45 |
| LBR | lamin B receptor | 143815 | 185842 | 154380 | |
| LAMB3 | laminin subunit beta 3 | 196878 | 008118 | 123689 | 13, 24, 35 |
| LAMC1 | laminin subunit gamma 1 | 135862 | 157060 | 058085 | 1, 26, 11 |
| LAMC2 | laminin subunit gamma 2 | 058085 | 135862 | 157064 | |
| LCE1A | late cornified envelope 1A | 186844 | 196734 | 235942 | 30, 35, |
| LCE1B | late cornified envelope 1B | 196734 | 197084 | 186844 | 35, |
| LCE1C | late cornified envelope 1C | 197084 | 172155 | 196734 | 35, 15, |
| LCE1D | late cornified envelope 1D | 172155 | 186226 | 197084 | 35, |
| LCE1E | late cornified envelope 1E | 186226 | 240386 | 172155 | |
| LCE1F | late cornified envelope 1F | 240386 | 203786 | 186226 | 35, |
| LCE2A | late cornified envelope 2A | 187173 | 159455 | 187170 | 10, 35, |
| LCE2B | late cornified envelope 2B | 159455 | 187180 | 187173 | 35, 15, 10 |
| LCE2C | late cornified envelope 2C | 187180 | 187223 | 159455 | 35, |
| LCE2D | late cornified envelope 2D | 187223 | 185962 | 187180 | 35, 17, |
| LCE3A | late cornified envelope 3A | 185962 | 187238 | 187223 | |
| LCE3B | late cornified envelope 3B | 187238 | 244057 | 185962 | |
| LCE3C | late cornified envelope 3C | 244057 | 163202 | 187238 | |
| LCE3D | late cornified envelope 3D | 163202 | 185966 | 244057 | |
| LCE3E | late cornified envelope 3E | 185966 | 169509 | 163202 | 4, 30, |
| LCE4A | late cornified envelope 4A | 187170 | 187173 | 198854 | 10, |
| LCE5A | late cornified envelope 5A | 186207 | 143536 | 279850 | 35, |
| LCE6A | late cornified envelope 6A | 235942 | 186844 | 163206 | 15, 10, 35 |
| LELP1 | late cornified envelope like proline rich 1 | 203784 | 159516 | 203783 | 38, 32, |
| LAMTOR2 | late endosomal/lysosomal adaptor, MAPK and MTOR activator 2 | 116586 | 160803 | 132698 | 43, 7, 1 |
| LAMTOR5 | late endosomal/lysosomal adaptor, MAPK and MTOR activator 5 | 134248 | 168679 | 143125 | 29, 15, 3 |
| LCK | LCK proto-oncogene, Src family tyrosine kinase | 182866 | 183615 | 116478 | 39, 41, 36 |
| LRP8 | LDL receptor related protein 8 | 157193 | 162385 | 143006 | 40, 3, 6 |
| LEFTY1 | left-right determination factor 1 | 243709 | 196187 | 255835 | 16, 9, 28 |
| LEFTY2 | left-right determination factor 2 | 143768 | 143811 | 143751 | 31, 38, 44 |
| LMOD1 | leiomodin 1 | 163431 | 198892 | 134375 | 42, 44, 26 |
| LEMD1 | LEM domain containing 1 | 186007 | 162873 | 117266 | |
| LENEP | lens epithelial protein | 163352 | 160688 | 160685 | 14, |
| LEPR | leptin receptor | 116678 | 213625 | 184588 | 42, 1, 7 |
| LEPROT | leptin receptor overlapping transcript | 213625 | 116675 | 116678 | 1, 29, 43 |
| LURAP1 | leucine rich adaptor protein 1 | 171357 | 085998 | 085999 | 29, 40, 8 |
| LINGO4 | leucine rich repeat and Ig domain containing 4 | 213171 | 182134 | 143365 | 14, 10, 17 |
| LRRC38 | leucine rich repeat containing 38 | 162494 | 204478 | 162493 | 14, 25, 32 |
| LRRC39 | leucine rich repeat containing 39 | 122477 | 122435 | 137992 | 25, 35, 15 |
| LRRC40 | leucine rich repeat containing 40 | 066557 | 033122 | 116754 | 43, 10, 5 |
| LRRC41 | leucine rich repeat containing 41 | 132128 | 085999 | 173660 | 39, 38, 45 |
| LRRC42 | leucine rich repeat containing 42 | 116212 | 081870 | 203985 | 29, 6, 9 |
| LRRC47 | leucine rich repeat containing 47 | 130764 | 235169 | 116198 | 27, 28, 3 |
| LRRC52 | leucine rich repeat containing 52 | 162763 | 143171 | 143198 | 38, 32, |
| LRRC53 | leucine rich repeat containing 53 | 162621 | 116783 | 178965 | |
| LRRC7 | leucine rich repeat containing 7 | 033122 | 024526 | 066557 | 18, 2, 15 |
| LRRC71 | leucine rich repeat containing 71 | 160838 | 187800 | 132694 | 38, 10, 20 |
| LRRC8B | leucine rich repeat containing 8 family member B | 197147 | 183347 | 171488 | 11, 38, 26 |
| LRRC8C | leucine rich repeat containing 8 family member C | 171488 | 197147 | 271949 | |
| LRRC8D | leucine rich repeat containing 8 family member D | 171492 | 271949 | 162664 | 26, 39, 44 |
| LGR6 | leucine rich repeat containing G protein-coupled receptor 6 | 133067 | 143851 | 077152 | 15, 27, 24 |
| LRRN2 | leucine rich repeat neuronal 2 | 170382 | 198625 | 163531 | 31, 27, 14 |
| LRIG2 | leucine rich repeats and immunoglobulin like domains 2 | 198799 | 155380 | 081026 | |
| LRRIQ3 | leucine rich repeats and IQ motif containing 3 | 162620 | 172260 | 254685 | 10, 38, 4 |
| LZIC | leucine zipper and CTNNBIP1 domain containing | 162441 | 178585 | 173614 | 42, 30, 43 |
| LUZP1 | leucine zipper protein 1 | 169641 | 004487 | 179546 | 16, 40, 24 |
| LRIF1 | ligand dependent nuclear receptor interacting factor 1 | 121931 | 143119 | 156171 | 22, 30, 24 |
| LMO4 | LIM domain only 4 | 143013 | 267561 | 065243 | 11, 18, 1 |
| LHX4 | LIM homeobox 4 | 121454 | 116260 | 230124 | 25, 44, |
| LHX8 | LIM homeobox 8 | 162624 | 162623 | 137968 | 35, 12, 10 |
| LHX9 | LIM homeobox 9 | 143355 | 203724 | 151414 | |
| LMX1A | LIM homeobox transcription factor 1 alpha | 162761 | 185630 | 143171 | 38, 17, 14 |
| LIX1L | limb and CNS expressed 1 like | 271601 | 280778 | 272031 | 29, 41, 26 |
| LIN28A | lin-28 homolog A | 131914 | 176083 | 117682 | 12, 38, 32 |
| LIN9 | lin-9 DREAM MuvB core complex component | 183814 | 185155 | 143799 | 39, 13, 22 |
| L1TD1 | LINE1 type transposase domain containing 1 | 240563 | 132849 | 132854 | 12, 32, 38 |
| LINC00083 | Long intergenic non-protein coding RNA 83 | 188585 | 075391 | 240021 | |
| LOR | loricrin | 203782 | 203783 | 159527 | 35, 10, |
| LDLRAP1 | low density lipoprotein receptor adaptor protein 1 | 157978 | 204178 | 117643 | 34, 35, 24 |
| LDLRAD1 | low density lipoprotein receptor class A domain containing 1 | 203985 | 116212 | 116209 | 20, 30, 15 |
| LDLRAD2 | low density lipoprotein receptor class A domain containing 2 | 187942 | 090686 | 142798 | 18, 4, 6 |
| LSM10 | LSM10, U7 small nuclear RNA associated | 181817 | 196182 | 116885 | 34, 29, 22 |
| LYPD8 | LY6/PLAUR domain containing 8 | 259823 | 189181 | 175137 | |
| LY9 | lymphocyte antigen 9 | 122224 | 026751 | 122223 | 36, 5, 39 |
| LEXM | lymphocyte expansion molecule | 162398 | 006555 | 116133 | 38, 6, 16 |
| LAX1 | lymphocyte transmembrane adaptor 1 | 122188 | 058668 | 058673 | 36, 30, 22 |
| KDM1A | lysine demethylase 1A | 004487 | 227868 | 169641 | 12, 23, 38 |
| KDM4A | lysine demethylase 4A | 066135 | 142949 | 126091 | 34, 44, 1 |
| KDM5B | lysine demethylase 5B | 117139 | 143858 | 183155 | 24, 38, 42 |
| LYSMD1 | LysM domain containing 1 | 163155 | 163156 | 163157 | 2, 39, 5 |
| LPAR3 | lysophosphatidic acid receptor 3 | 171517 | 117155 | 153898 | |
| LPGAT1 | lysophosphatidylglycerol acyltransferase 1 | 123684 | 117650 | 143493 | 1, 43, 22 |
| LYPLA2 | lysophospholipase II | 011009 | 057757 | 117308 | 41, 21, 2 |
| LYPLAL1 | lysophospholipase like 1 | 143353 | 092969 | 215817 | 1, 22, 15 |
| LAPTM5 | lysosomal protein transmembrane 5 | 162511 | 162510 | 162512 | 41, 22, 5 |
| LYST | lysosomal trafficking regulator | 143669 | 168243 | 116962 | 5, 40, 39 |
| MAB21L3 | mab-21 like 3 | 173212 | 163393 | 163399 | 17, 44, |
| MAD2L2 | MAD2 mitotic arrest deficient-like 2 (yeast) | 116670 | 116663 | 162490 | 21, 34, 1 |
| MAEL | maelstrom spermatogenic transposon silencer | 143194 | 143195 | 143167 | 38, 32, 8 |
| MROH7 | maestro heat like repeat family member 7 | 184313 | 162391 | 271723 | 38, 40, 41 |
| MROH9 | maestro heat like repeat family member 9 | 117501 | 116132 | 007933 | 42, 38, 20 |
| MAGOH | mago homolog, exon junction complex core component | 162385 | 162384 | 157193 | 11, 3, 30 |
| MFSD14A | major facilitator superfamily domain containing 14A | 156875 | 117620 | 156876 | 11, 9, 29 |
| MFSD2A | major facilitator superfamily domain containing 2A | 168389 | 116990 | 131236 | 32, 38, 33 |
| MFSD4A | major facilitator superfamily domain containing 4A | 174514 | 117266 | 158711 | 42, 8, 18 |
| MR1 | major histocompatibility complex, class I-related | 153029 | 135823 | 162783 | 31, 24, 42 |
| MASP2 | mannan binding lectin serine peptidase 2 | 009724 | 277726 | 116649 | 19, 40, 18 |
| MAN1A2 | mannosidase alpha class 1A member 2 | 198162 | 134258 | 183508 | 30, 32, 11 |
| MAN1C1 | mannosidase alpha class 1C member 1 | 117643 | 157978 | 162430 | 32, 1, 29 |
| MANEAL | mannosidase endo-alpha like | 185090 | 183317 | 196449 | |
| MKNK1 | MAP kinase interacting serine/threonine kinase 1 | 079277 | 162456 | 142961 | 18, 39, 36 |
| MAP7D1 | MAP7 domain containing 1 | 116871 | 054116 | 054118 | 26, 34, 12 |
| MARCKSL1 | MARCKS like 1 | 175130 | 116478 | 162526 | 41, 43, 21 |
| MATN1 | matrilin 1, cartilage matrix protein | 162510 | 060656 | 162511 | 42, 12, 32 |
| MMP23B | matrix metallopeptidase 23B | 189409 | 197530 | 248333 | 28, |
| MXRA8 | matrix remodeling associated 8 | 162576 | 107404 | 175756 | 10, 1, 45 |
| MDM4 | MDM4, p53 regulator | 198625 | 133056 | 170382 | 39, 18, 40 |
| MTOR | mechanistic target of rapamycin | 198793 | 171824 | 171819 | 22, 1, 35 |
| MED18 | mediator complex subunit 18 | 130772 | 130766 | 204138 | 21, 30, 41 |
| MED8 | mediator complex subunit 8 | 159479 | 066322 | 198198 | 37, 27, 5 |
| MAGI3 | membrane associated guanylate kinase, WW and PDZ domain containing 3 | 081026 | 198799 | 116793 | 14, 11, 31 |
| MMEL1 | membrane metalloendopeptidase like 1 | 142606 | 157870 | 215912 | 1, 38, 3 |
| MTF1 | metal regulatory transcription factor 1 | 188786 | 197982 | 204084 | 20, 9, 1 |
| MTF2 | metal response element binding transcription factor 2 | 143033 | 154511 | 117500 | 40, 22, 34 |
| MT1HL1 | metallothionein 1H-like 1 | 244020 | 116984 | 198626 | |
| MTX1 | metaxin 1 | 173171 | 169231 | 177628 | 34, 13, 20 |
| MTHFR | methylenetetrahydrofolate reductase | 177000 | 215910 | 011021 | |
| MMACHC | methylmalonic aciduria (cobalamin deficiency) cblC type, with homocystinuria | 132763 | 280670 | 117450 | 21, 6, 19 |
| METTL11B | methyltransferase like 11B | 203740 | 075945 | 120370 | |
| METTL13 | methyltransferase like 13 | 010165 | 117533 | 197959 | 3, 42, 24 |
| METTL18 | methyltransferase like 18 | 171806 | 007908 | 000457 | 29, 31, 3 |
| MEX3A | mex-3 RN A binding family member A | 254726 | 132698 | 160789 | 14, 12, 27 |
| MIA3 | MIA family member 3, ER export factor | 154305 | 143498 | 186063 | 11, 39, 30 |
| MFAP2 | microfibrillar associated protein 2 | 117122 | 058453 | 159363 | 15, 6, 12 |
| MGST3 | microsomal glutathione S-transferase 3 | 143198 | 162763 | 143149 | |
| MARK1 | microtubule affinity regulating kinase 1 | 116141 | 118873 | 162817 | 4, 26, 24 |
| MAP1LC3C | microtubule associated protein 1 light chain 3 gamma | 197769 | 196289 | 180287 | 12, |
| MAP10 | microtubule associated protein 10 | 212916 | 116991 | 135778 | 31, 39, 38 |
| MAST2 | microtubule associated serine/threonine kinase 2 | 086015 | 197429 | 117461 | 22, 26, 16 |
| MACF1 | microtubule-actin crosslinking factor 1 | 127603 | 168653 | 183682 | |
| MIER1 | MIER1 transcriptional regulator | 198160 | 152763 | 116704 | 26, 22, 7 |
| MIIP | migration and invasion inhibitory protein | 116691 | 116688 | 120949 | 1, 9, 36 |
| MIB2 | mindbomb E3 ubiquitin protein ligase 2 | 197530 | 228594 | 189409 | 28, 9, 39 |
| MINOS1-NBL1 | MINOS1-NBL1 readthrough | 270136 | 173436 | 158747 | |
| MSTO1 | misato 1, mitochondrial distribution and morphology regulator | 125459 | 116539 | 163374 | 1, 13, 35 |
| MARC1 | mitochondrial amidoxime reducing component 1 | 186205 | 117791 | 136630 | |
| MARC2 | mitochondrial amidoxime reducing component 2 | 117791 | 162817 | 186205 | 29, 30, 17 |
| MUL1 | mitochondrial E3 ubiquitin protein ligase 1 | 090432 | 162545 | 183114 | 36, 38, 41 |
| MTFR1L | mitochondrial fission regulator 1 like | 117640 | 255054 | 127423 | 15, 2, 38 |
| MINOS1 | mitochondrial inner membrane organizing system 1 | 173436 | 077549 | 270136 | 15, 3, 25 |
| MPC2 | mitochondrial pyruvate carrier 2 | 143158 | 143199 | 143164 | 43, 33, 41 |
| MRPL20 | mitochondrial ribosomal protein L20 | 242485 | 221978 | 235098 | 3, 31, 13 |
| MRPL24 | mitochondrial ribosomal protein L24 | 143314 | 143303 | 143321 | 29, 3, 35 |
| MRPL37 | mitochondrial ribosomal protein L37 | 116221 | 215883 | 157216 | 9, 41, 1 |
| MRPL55 | mitochondrial ribosomal protein L55 | 162910 | 143793 | 143774 | 29, 3, 15 |
| MRPL9 | mitochondrial ribosomal protein L9 | 143436 | 178796 | 143450 | 29, 14, 41 |
| MRPS14 | mitochondrial ribosomal protein S14 | 120333 | 116161 | 120332 | |
| MRPS15 | mitochondrial ribosomal protein S15 | 116898 | 116885 | 119535 | 43, 7, 27 |
| MRPS21 | mitochondrial ribosomal protein S21 | 266472 | 159208 | 278939 | 29, 7, 10 |
| MECR | mitochondrial trans-2-enoyl-CoA reductase | 116353 | 116350 | 060656 | 41, 35, 27 |
| MFN2 | mitofusin 2 | 116688 | 083444 | 116691 | 42, 40, 8 |
| MAP3K6 | mitogen-activated protein kinase kinase kinase 6 | 142733 | 142765 | 142748 | 34, 37, 26 |
| MAPKAPK2 | mitogen-activated protein kinase-activated protein kinase 2 | 162889 | 143479 | 136634 | 34, 18, 6 |
| MIGA1 | mitoguardin 1 | 180488 | 077254 | 162614 | 18, 45, 26 |
| MIXL1 | Mix paired-like homeobox | 185155 | 182827 | 183814 | 10, |
| MOB3C | MOB kinase activator 3C | 142961 | 079277 | 123472 | 1, 29, 14 |
| MORN1 | MORN repeat containing 1 | 116151 | 157933 | 157916 | 20, 34, 29 |
| MOV10 | Mov10 RISC complex RNA helicase | 155363 | 116489 | 155366 | 39, 43, 20 |
| MPL | MPL proto-oncogene, thrombopoietin receptor | 117400 | 066056 | 117399 | 38, 20, |
| MROH7-TTC4 | MROH7-TTC4 readthrough (NMD candidate) | 271723 | 184313 | 243725 | 38, 40, 41 |
| MRTO4 | MRT4 homolog, ribosome maturation factor | 053372 | 127463 | 211454 | 21, 35, 3 |
| MTRNR2L11 | MT-RNR2-like 11 | 270188 | 116996 | 133019 | |
| MUC1 | mucin 1, cell surface associated | 185499 | 163462 | 279986 | 37, 3, 23 |
| MCOLN2 | mucolipin 2 | 153898 | 171517 | 162643 | 13, 22, 36 |
| MCOLN3 | mucolipin 3 | 055732 | 162643 | 097096 | 29, 31, 2 |
| MEGF6 | multiple EGF like domains 6 | 162591 | 130762 | 158109 | 18, 15, 1 |
| MSH4 | mutS homolog 4 | 057468 | 137955 | 154007 | 10, 38, 24 |
| MUTYH | mutY DNA glycosylase | 132781 | 186603 | 132773 | 37, 21, 26 |
| MYSM1 | Myb like, SWIRM and MPN domains 1 | 162601 | 184292 | 177606 | 33, 30, 7 |
| MYCBP | MYC binding protein | 214114 | 116954 | 274944 | 3, 38, 32 |
| MPZ | myelin protein zero | 158887 | 248485 | 143252 | 26, 11, 31 |
| MPZL1 | myelin protein zero like 1 | 197965 | 198771 | 143199 | 24, 3, 11 |
| MDS2 | myelodysplastic syndrome 2 translocation associated | 197880 | 117318 | 142676 | 16, 20, 38 |
| MNDA | myeloid cell nuclear differentiation antigen | 163563 | 188340 | 163564 | 5, 40, 32 |
| MLLT11 | myeloid/lymphoid or mixed-lineage leukemia; translocated to, 11 | 213190 | 197622 | 143458 | 7, 45, 26 |
| MYOC | myocilin | 034971 | 283683 | 117533 | 42, 13, 14 |
| MEF2D | myocyte enhancer factor 2D | 116604 | 125462 | 183856 | 22, 14, 11 |
| MYOG | myogenin | 122180 | 143847 | 163485 | 26, 25, 14 |
| MYOM3 | myomesin 3 | 142661 | 188529 | 142677 | 15, 25, 18 |
| MYBPH | myosin binding protein H | 133055 | 163485 | 133048 | 29, 15, 24 |
| MYBPHL | myosin binding protein H like | 221986 | 134222 | 134243 | 27, 14, 37 |
| MTMR11 | myotubularin related protein 11 | 014914 | 143368 | 264522 | 18, 2, 16 |
| MEAF6 | MYST/Esa1 associated factor 6 | 163875 | 163874 | 163877 | 45, 34, 9 |
| NPL | N-acetylneuraminate pyruvate lyase | 135838 | 135824 | 135829 | 24, 17, 29 |
| NADK | NAD kinase | 008130 | 215790 | 078369 | 1, 22, 29 |
| NAXE | NAD(P)HX epimerase | 163382 | 187862 | 160818 | |
| NDUFS2 | NADH: ubiquinone oxidoreductase core subunit S2 | 158864 | 158859 | 158869 | 26, 35, 30 |
| NDUFS5 | NADH: ubiquinone oxidoreductase subunit S5 | 168653 | 174574 | 127603 | 41, 29, 25 |
| NRDC | nardilysin convertase | 078618 | 117859 | 169213 | 42, 38, 29 |
| NPPA | natriuretic peptide A | 175206 | 011021 | 120937 | 15, 20, 10 |
| NPPB | natriuretic peptide B | 120937 | 175206 | 116685 | 15, 12, 28 |
| NPR1 | natriuretic peptide receptor 1 | 169418 | 143621 | 143624 | 23, 1, 26 |
| NDC1 | NDC1 transmembrane nudeoporin | 058804 | 174332 | 058799 | 22, 38, 42 |
| NECAP2 | NECAP endocytosis associated 2 | 157191 | 187144 | 268991 | 34, 43, 41 |
| NECTIN4 | nectin cell adhesion molecule 4 | 143217 | 278883 | 162755 | 24, 42, 18 |
| NPHP4 | nephrocystin 4 | 131697 | 196581 | 069424 | 3, 25, 26 |
| NGF | nerve growth factor | 134259 | 134198 | 173218 | 36, 20, 35 |
| NHLH1 | nescient helix-loop-helix 1 | 171786 | 162736 | 162738 | 31, 20, 14 |
| NHLH2 | nescient helix-loop-helix 2 | 177551 | 118729 | 163393 | 38, 8, 24 |
| NES | nestin | 132688 | 132692 | 143320 | 12, 15, 27 |
| NTNG1 | netrin G1 | 162631 | 198890 | 134215 | 26, 8, 17 |
| NENF | neudesin neurotrophic factor | 117691 | 065600 | 162772 | 24, 15, 20 |
| NBL1 | neuroblastoma 1, DAN family BMP antagonist | 158747 | 270136 | 158748 | 34, 13, 3 |
| NBPF1 | neuroblastoma breakpoint family member 1 | 219481 | 268991 | 237847 | |
| NBPF10 | neuroblastoma breakpoint family member 10 | 271425 | 168509 | 213240 | |
| NBPF11 | neuroblastoma breakpoint family member 11 | 263956 | 188092 | 280649 | 18, 11, 45 |
| NBPF12 | neuroblastoma breakpoint family member 12 | 268043 | 270339 | 131791 | |
| NBPF14 | neuroblastoma breakpoint family member 14 | 270629 | 236334 | 178104 | 23, 33, |
| NBPF15 | neuroblastoma breakpoint family member 15 | 266338 | 271567 | 279782 | 40, 18, 26 |
| NBPF19 | neuroblastoma breakpoint family member 19 | 271383 | 269713 | 263464 | 34, 23, 9 |
| NBPF20 | neuroblastoma breakpoint family member 20 | 162825 | 256374 | 117262 | |
| NBPF26 | neuroblastoma breakpoint family member 26 | 273136 | 265808 | 263353 | |
| NBPF3 | neuroblastoma breakpoint family member 3 | 142794 | 117298 | 162551 | 38, 42, 13 |
| NBPF4 | neuroblastoma breakpoint family member 4 | 196427 | 085491 | 186086 | 38, |
| NBPF6 | neuroblastoma breakpoint family member 6 | 186086 | 196427 | 162636 | 38, |
| NBPF9 | neuroblastoma breakpoint family member 9 | 269713 | 178104 | 271383 | |
| NRAS | neuroblastoma RAS viral oncogene homolog | 213281 | 116748 | 009307 | 26, 22, 7 |
| NCDN | neurochondrin | 020129 | 142687 | 116819 | 34, 35, 40 |
| NFASC | neurofascin | 163531 | 170382 | 184144 | 26, 8, 11 |
| NAV1 | neuron navigator 1 | 134369 | 159176 | 198700 | 24, 43, 1 |
| NEGR1 | neuronal growth regulator 1 | 172260 | 132485 | 162620 | 1, 42, 10 |
| NTRK1 | neurotrophic receptor tyrosine kinase 1 | 198400 | 027869 | 027644 | 26, 7, 2 |
| NCF2 | neutrophil cytosolic factor 2 | 116701 | 116698 | 162704 | 5, 36, 10 |
| NEXN | nexilin F-actin binding protein | 162614 | 180488 | 162613 | 25, 15, 24 |
| NCSTN | nicastrin | 162736 | 122218 | 171786 | 40, 24, 38 |
| NMNAT1 | nicotinamide nucleotide adenylyltransferase 1 | 173614 | 162441 | 162444 | 43, 34, 15 |
| NMNAT2 | nicotinamide nucleotide adenylyltransferase 2 | 157064 | 058085 | 116698 | 11, 8, 31 |
| NID1 | nidogen 1 | 116962 | 143669 | 077585 | 11, 28, 15 |
| NEK2 | NIMA related kinase 2 | 117650 | 170385 | 123684 | 4, 3, 6 |
| NEK7 | NIMA related kinase 7 | 151414 | 143355 | 151418 | 40, 32, 43 |
| NIPAL3 | NIPAIike domain containing 3 | 001461 | 001460 | 117602 | 29, 8, 24 |
| NOS1AP | nitric oxide synthase 1 adaptor protein | 198929 | 162745 | 254706 | 23, 38, 17 |
| NIT1 | nitrilase 1 | 158793 | 143256 | 158796 | 2, 34, 22 |
| NLRP3 | NLR family pyrin domain containing 3 | 162711 | 162714 | 177535 | 39, 7, 10 |
| NME7 | NME/NM23 family member 7 | 143156 | 143153 | 117475 | 10, 43, 31 |
| NOC2L | NOC2 like nucleolar associated transcriptional repressor | 188976 | 187634 | 187961 | 18, 3, 9 |
| NCMAP | non-compact myelin associated protein | 184454 | 117602 | 133226 | 40, 26, 32 |
| NSUN4 | NOP2/Sun RNA methyltransferase family member 4 | 117481 | 173660 | 117480 | 18, 43, 38 |
| NOTCH2 | notch 2 | 134250 | 134249 | 265808 | 23, 11, 5 |
| NOTCH2NL | notch 2 N-terminal like | 264343 | 213240 | 270339 | |
| NPHS2 | NPHS2, podocin | 116218 | 162779 | 162782 | 17, |
| NSL1 | NSL1, MIS12 kinetochore complex component | 117697 | 123685 | 203705 | 29, 34, 22 |
| NUAK2 | NUAK family kinase 2 | 163545 | 133069 | 162873 | 21, 31, 42 |
| NASP | nuclear autoantigenic sperm protein | 132780 | 117448 | 159588 | 21, 12, 41 |
| NUCKS1 | nuclear casein kinase and cyclin dependent kinase substrate 1 | 069275 | 158715 | 117280 | 27, 11, 41 |
| NUDC | nuclear distribution C, dynein complex regulator | 090273 | 198746 | 131910 | 35, 12, 34 |
| NFIA | nuclear factor IA | 162599 | 162598 | 162604 | |
| NR0B2 | nuclear receptor subfamily 0 group B member 2 | 131910 | 090273 | 175707 | 15, 19, 37 |
| NR1I3 | nuclear receptor subfamily 1 group I member 3 | 143257 | 158882 | 248485 | 19, 17, 25 |
| NR5A2 | nuclear receptor subfamily 5 group A member 2 | 116833 | 081237 | 162702 | 26, 28, 42 |
| NFYC | nuclear transcription factor Y subunit gamma | 066136 | 117016 | 117013 | 43, 37, 36 |
| NVL | nuclear VCP-like | 143748 | 143753 | 143771 | 13, 39, 24 |
| NOL9 | nucleolar protein 9 | 162408 | 171680 | 173662 | 41, 37, 22 |
| NUP133 | nudeoporin 133 | 069248 | 143632 | 135776 | 42, 10, 24 |
| NUP210L | nudeoporin 210 like | 143552 | 177954 | 143549 | 38, 22, 12 |
| NTPCR | nudeoside-triphosphatase, cancer-related | 135778 | 212916 | 135749 | 43, 10, 41 |
| NUDT17 | nudix hydrolase 17 | 186364 | 186141 | 131788 | 27, 11, 29 |
| NUF2 | NUF2, NDC80 kinetochore complex component | 143228 | 143248 | 185630 | 34, 38, 39 |
| OBSCN | obscurin, cytoskeletal calmodulin and titin-interacting RhoGEF | 154358 | 162913 | 154370 | 18, 25, 40 |
| OCLM | oculomedin | 262180 | 279374 | 116703 | 12, |
| OLFM3 | olfactomedin 3 | 118733 | 170989 | 060718 | 8, 6, 17 |
| OLFML2B | olfactomedin like 2B | 162745 | 118217 | 198929 | |
| OLFML3 | olfactomedin like 3 | 116774 | 163349 | 134207 | 43, 9, 1 |
| OR1C1 | olfactory receptor family 1 subfamily C member 1 | 221888 | 153230 | 196772 | |
| OR10J1 | olfactory receptor family 10 subfamily J member 1 | 196184 | 249730 | 184155 | 38, |
| OR10J3 | olfactory receptor family 10 subfamily J member 3 | 196266 | 179639 | 249730 | |
| OR10J4 | olfactory receptor family 10 subfamily J member 4 (gene/pseudogene) | 249730 | 196266 | 196184 | |
| OR10J5 | olfactory receptor family 10 subfamily J member 5 | 184155 | 196184 | 132703 | |
| OR10K1 | olfactory receptor family 10 subfamily K member 1 | 173285 | 180708 | 198965 | |
| OR10K2 | olfactory receptor family 10 subfamily K member 2 | 180708 | 186306 | 173285 | |
| OR10R2 | olfactory receptor family 10 subfamily R member 2 | 198965 | 173285 | 197532 | 20, |
| OR10T2 | olfactory receptor family 10 subfamily T member 2 | 186306 | 158488 | 180708 | |
| OR10X1 | olfactory receptor family 10 subfamily X member 1 (gene/pseudogene) | 279111 | 186440 | 198967 | |
| OR10Z1 | olfactory receptor family 10 subfamily Z member 1 | 198967 | 279111 | 163554 | |
| OR11L1 | olfactory receptor family 11 subfamily L member 1 | 197591 | 196772 | 162722 | |
| OR13G1 | olfactory receptor family 13 subfamily G member 1 | 197437 | 177476 | 169214 | |
| OR14A16 | olfactory receptor family 14 subfamily A member 16 | 196772 | 221888 | 197591 | |
| OR14A2 | Olfactory receptor family 14 subfamily A member 2 | 241128 | 169214 | 153230 | |
| OR14C36 | olfactory receptor family 14 subfamily C member 36 | 177174 | 177186 | 196944 | |
| OR14I1 | olfactory receptor family 14 subfamily I member 1 | 189181 | 187701 | 259823 | |
| OR14K1 | Olfactory receptor family 14 subfamily K member 1 | 153230 | 241128 | 221888 | |
| OR2AJ1 | Olfactory receptor family 2 subfamily AJ member 1 | 177275 | 177462 | 279263 | |
| OR2AK2 | olfactory receptor family 2 subfamily AK member 2 | 187080 | 279263 | 197454 | |
| OR2B11 | olfactory receptor family 2 subfamily B member 11 | 177535 | 162711 | 169224 | 18, |
| OR2C3 | olfactory receptor family 2 subfamily C member 3 | 196242 | 169224 | 177489 | |
| OR2G2 | olfactory receptor family 2 subfamily G member 2 | 177489 | 196242 | 177476 | |
| OR2G3 | olfactory receptor family 2 subfamily G member 3 | 177476 | 177489 | 197437 | |
| OR2G6 | olfactory receptor family 2 subfamily G member 6 | 188558 | 203661 | 182783 | |
| OR2L13 | olfactory receptor family 2 subfamily L member 13 | 196071 | 198128 | 162727 | 8, |
| OR2L2 | olfactory receptor family 2 subfamily L member 2 | 203663 | 197454 | 198128 | |
| OR2L3 | olfactory receptor family 2 subfamily L member 3 | 198128 | 203663 | 196071 | |
| OR2L5 | olfactory receptor family 2 subfamily L member 5 | 197454 | 187080 | 203663 | |
| OR2L8 | olfactory receptor family 2 subfamily L member 8 (gene/pseudogene) | 279263 | 177275 | 187080 | |
| OR2M2 | olfactory receptor family 2 subfamily M member 2 | 198601 | 162727 | 228198 | |
| OR2M3 | olfactory receptor family 2 subfamily M member 3 | 228198 | 198601 | 171180 | |
| OR2M4 | olfactory receptor family 2 subfamily M member 4 | 171180 | 228198 | 177212 | |
| OR2M5 | olfactory receptor family 2 subfamily M member 5 | 162727 | 196071 | 198601 | |
| OR2M7 | olfactory receptor family 2 subfamily M member 7 | 177186 | 177201 | 177174 | |
| OR2T1 | olfactory receptor family 2 subfamily T member 1 | 175143 | 198104 | 196240 | |
| OR2T10 | olfactory receptor family 2 subfamily T member 10 | 184022 | 183310 | 279301 | |
| OR2T11 | olfactory receptor family 2 subfamily T member 11 (gene/pseudogene) | 279301 | 184022 | 177151 | |
| OR2T12 | olfactory receptor family 2 subfamily T member 12 | 177201 | 177212 | 177186 | |
| OR2T2 | olfactory receptor family 2 subfamily T member 2 | 196240 | 175143 | 196539 | |
| OR2T27 | olfactory receptor family 2 subfamily T member 27 | 187701 | 177151 | 189181 | |
| OR2T29 | olfactory receptor family 2 subfamily T member 29 | 182783 | 188558 | 183310 | |
| OR2T3 | olfactory receptor family 2 subfamily T member 3 | 196539 | 196240 | 203661 | |
| OR2T33 | olfactory receptor family 2 subfamily T member 33 | 177212 | 171180 | 177201 | |
| OR2T34 | olfactory receptor family 2 subfamily T member 34 | 183310 | 182783 | 184022 | |
| OR2T35 | olfactory receptor family 2 subfamily T member 35 | 177151 | 279301 | 187701 | |
| OR2T4 | olfactory receptor family 2 subfamily T member 4 | 196944 | 177174 | 198104 | |
| OR2T5 | olfactory receptor family 2 subfamily T member 5 | 203661 | 196539 | 188558 | |
| OR2T6 | olfactory receptor family 2 subfamily T member 6 | 198104 | 196944 | 175143 | |
| OR2T8 | olfactory receptor family 2 subfamily T member 8 | 177462 | 238243 | 177275 | |
| OR2W3 | olfactory receptor family 2 subfamily W member 3 | 238243 | 162722 | 177462 | |
| OR4F16 | olfactory receptor family 4 subfamily F member 16 | 273547 | 278566 | 187634 | |
| OR4F29 | olfactory receptor family 4 subfamily F member 29 | 278566 | 279457 | 273547 | |
| OR4F5 | olfactory receptor family 4 subfamily F member 5 | 186092 | 064703 | 279928 | |
| OR6F1 | olfactory receptor family 6 subfamily F member 1 | 169214 | 197437 | 241128 | 38, |
| OR6K2 | olfactory receptor family 6 subfamily K member 2 | 196171 | 163554 | 203757 | |
| OR6K3 | olfactory receptor family 6 subfamily K member 3 | 203757 | 196171 | 180433 | |
| OR6K6 | olfactory receptor family 6 subfamily K member 6 | 180433 | 203757 | 197403 | |
| OR6N1 | olfactory receptor family 6 subfamily N member 1 | 197403 | 180433 | 188340 | |
| OR6N2 | olfactory receptor family 6 subfamily N member 2 | 188340 | 197403 | 163563 | |
| OR6P1 | olfactory receptor family 6 subfamily P member 1 | 186440 | 197532 | 279111 | |
| OR6Y1 | olfactory receptor family 6 subfamily Y member 1 | 197532 | 198965 | 186440 | |
| OMA1 | OMA1 zinc metallopeptidase | 162600 | 173406 | 184292 | 10, 22, 33 |
| OPRD1 | opioid receptor delta 1 | 116329 | 198492 | 159023 | 27, 8, |
| OPN3 | opsin 3 | 054277 | 117009 | 203668 | 9, 32, 2 |
| OPTC | opticin | 188770 | 188783 | 058668 | 14, 8, 32 |
| OSCP1 | organic solute carrier partner 1 | 116885 | 181817 | 116898 | 30, 29, 1 |
| ORC1 | origin recognition complex subunit 1 | 085840 | 154222 | 134748 | 39, 7, 21 |
| OAZ3 | ornithine decarboxylase antizyme 3 | 143450 | 143436 | 182134 | 38, 29, 45 |
| OTUD3 | OTU deubiquitinase 3 | 169914 | 178828 | 188784 | 26, 18, 14 |
| OTUD7B | OTU deubiquitinase 7B | 264522 | 014914 | 136631 | 4, 1, 26 |
| ODF2L | outer dense fiber of sperm tails 2 like | 122417 | 171502 | 137975 | 4, 30, 29 |
| OVGP1 | oviductal glycoprotein 1 | 085465 | 173947 | 116455 | 27, 29, 44 |
| OSBPL9 | oxysterol binding protein like 9 | 117859 | 085832 | 078618 | 36, 24, 4 |
| PAX7 | paired box 7 | 009709 | 179023 | 179002 | 14, 10, 8 |
| PRRX1 | paired related homeobox 1 | 116132 | 120370 | 117501 | 26, 11, 15 |
| PALMD | palmdelphin | 099260 | 117600 | 156869 | 43, 15, 26 |
| PPT1 | palmitoyl-protein thioesterase 1 | 131238 | 131236 | 117000 | 30, 13, 36 |
| PANK4 | pantothenate kinase 4 | 157881 | 149527 | 197921 | 26, 39, 7 |
| PRCC | papillary renal cell carcinoma (translocation-associated) | 143294 | 143321 | 027869 | 13, 9, 14 |
| PAPPA2 | pappalysin 2 | 116183 | 143207 | 152092 | 32, 44, 14 |
| PARK7 | Parkinsonism associated deglycase | 116288 | 049249 | 116285 | 3, 31, 29 |
| PTCH2 | patched 2 | 117425 | 222009 | 070785 | 38, 5, 8 |
| PATJ | PATJ, crumbs cell polarity complex component | 132849 | 162604 | 240563 | 42, 24, 23 |
| PBX1 | PBX homeobox 1 | 185630 | 143228 | 162761 | 33, 23, 38 |
| PBXIP1 | PBX homeobox interacting protein 1 | 163346 | 163344 | 163348 | 39, 24, 42 |
| PDIK1L | PDLIM1 interacting kinase 1 like | 175087 | 158022 | 197245 | 11, 31, 22 |
| PDZK1 | PDZ domain containing 1 | 174827 | 117262 | 117281 | 17, 19, 26 |
| PDZK1IP1 | PDZK1 interacting protein 1 | 162366 | 162365 | 162367 | 17, 30, 37 |
| PCNX2 | pecanex homolog 2 (Drosophila) | 135749 | 135778 | 143674 | 24, 36, 40 |
| PEF1 | penta-EF-hand domain containing 1 | 162517 | 121764 | 084636 | 32, 41, 34 |
| PM20D1 | peptidase M20 domain containing 1 | 162877 | 133065 | 174502 | 35, 36, 22 |
| PGLYRP3 | peptidoglycan recognition protein 3 | 159527 | 203782 | 163218 | 35, |
| PGLYRP4 | peptidoglycan recognition protein 4 | 163218 | 159527 | 163220 | 13, 15, 25 |
| PADI1 | peptidyl arginine deiminase 1 | 142623 | 117115 | 142619 | 24, 13, 42 |
| PADI2 | peptidyl arginine deiminase 2 | 117115 | 117118 | 142623 | 18, 5, 14 |
| PADI3 | peptidyl arginine deiminase 3 | 142619 | 142623 | 159339 | 30, 24, 8 |
| PADI4 | peptidyl arginine deiminase 4 | 159339 | 142619 | 280226 | 5, 7, 36 |
| PADI6 | peptidyl arginine deiminase 6 | 276747 | 280226 | 179051 | 35, |
| PPIAL4A | peptidylprolyl isomerase A like 4A | 263353 | 273136 | 281741 | |
| PPIAL4C | peptidylprolyl isomerase A like 4C | 263464 | 271383 | 272058 | |
| PPIAL4D | peptidylprolyl isomerase A like 4D | 256374 | 281571 | 162825 | |
| PPIAL4E | peptidylprolyl isomerase A like 4E | 271567 | 263513 | 266338 | |
| PPIAL4F | peptidylprolyl isomerase A like 4F | 279782 | 266338 | 196369 | |
| PPIAL4G | peptidylprolyl isomerase A like 4G | 236334 | 280649 | 270629 | 38, |
| PPIE | peptidylprolyl isomerase E | 084072 | 116983 | 116985 | 34, 30, 41 |
| PPIH | peptidylprolyl isomerase H | 171960 | 186409 | 065978 | 43, 36, 2 |
| PER3 | period circadian clock 3 | 049246 | 049245 | 049247 | 40, 36, 14 |
| PRDX1 | peroxiredoxin 1 | 117450 | 132763 | 117448 | 4, 3, 34 |
| PRDX6 | peroxiredoxin 6 | 117592 | 117586 | 162753 | 7, 34, 9 |
| PEX10 | peroxisomal biogenesis factor 10 | 157911 | 157916 | 149527 | 26, 33, 38 |
| PEX11B | peroxisomal biogenesis factor 11 beta | 131779 | 143127 | 265241 | 34, 30, 31 |
| PEX14 | peroxisomal biogenesis factor 14 | 142655 | 160049 | 130940 | 31, 29, 14 |
| PEX19 | peroxisomal biogenesis factor 19 | 162735 | 258465 | 122218 | 42, 40, 1 |
| PHF13 | PHD finger protein 13 | 116273 | 162413 | 041988 | 31, 38, 22 |
| PDC | phosducin | 116703 | 262180 | 073756 | 14, 25, |
| PHACTR4 | phosphatase and actin regulator 4 | 204138 | 130772 | 180198 | 43, 2, 9 |
| PI4KB | phosphatidylinositol 4-kinase beta | 143393 | 143373 | 143390 | 31, 40, 18 |
| PIGC | phosphatidylinositol glycan anchor biosynthesis class C | 135845 | 197959 | 180999 | 1, 4, 7 |
| PIGK | phosphatidylinositol glycan anchor biosynthesis class K | 142892 | 117069 | 154027 | 10, 13, 29 |
| PIGM | phosphatidylinositol glycan anchor biosynthesis class M | 143315 | 177807 | 162728 | 39, 45, 11 |
| PIGV | phosphatidylinositol glycan anchor biosynthesis class V | 060642 | 117713 | 204160 | 38, 13, 9 |
| PIK3CD | phosphatidylinositol-4,5-bisphosphate 3-kinase catalytic subunit delta | 171608 | 188807 | 171603 | 22, 21, 43 |
| PIK3C2B | phosphatidylinositol-4-phosphate 3-kinase catalytic subunit type 2 beta | 133056 | 158615 | 198625 | 22, 32, 16 |
| PIP5K1A | phosphatidylinositol-4-phosphate 5-kinase type 1 alpha | 143398 | 163159 | 159352 | |
| PDE4B | phosphodiesterase 4B | 184588 | 116678 | 118473 | 42, 8, 40 |
| PDE4DIP | phosphodiesterase 4D interacting protein | 178104 | 270629 | 269713 | |
| PGM1 | phosphoglucomutase 1 | 079739 | 203965 | 185483 | 15, 11, 34 |
| PGD | phosphogluconate dehydrogenase | 142657 | 054523 | 251503 | 13, 5, 24 |
| PHGDH | phosphoglycerate dehydrogenase | 092621 | 143067 | 134240 | 21, 9, 3 |
| PIK3R3 | phosphoinositide-3-kinase regulatory subunit 3 | 117461 | 086015 | 278139 | 4, 40, 25 |
| PLA2G2A | phospholipase A2 group IIA | 188257 | 188784 | 127472 | 1, 10, 32 |
| PLA2G2C | phospholipase A2 group IIC | 187980 | 158786 | 162543 | |
| PLA2G2D | phospholipase A2 group IID | 117215 | 127472 | 158786 | 10, 36, 30 |
| PLA2G2E | phospholipase A2 group IIE | 188784 | 169914 | 188257 | |
| PLA2G2F | phospholipase A2 group IIF | 158786 | 117215 | 187980 | 39, |
| PLA2G4A | phospholipase A2 group IVA | 116711 | 073756 | 162670 | 30, 10, 44 |
| PLA2G5 | phospholipase A2 group V | 127472 | 188257 | 117215 | 14, 32, 2 |
| PLCH2 | phospholipase C eta 2 | 149527 | 157911 | 157881 | 31, 14, 22 |
| PLD5 | phospholipase D family member 5 | 180287 | 197769 | 143702 | 42, 28, 14 |
| PLPP3 | phospholipid phosphatase 3 | 162407 | 162402 | 162409 | |
| PLPPR4 | phospholipid phosphatase related 4 | 117600 | 117598 | 099260 | 31, 8, 24 |
| PLPPR5 | phospholipid phosphatase related 5 | 117598 | 162627 | 117600 | 10, 28, 45 |
| PMVK | phosphomevalonate kinase | 163344 | 143603 | 163346 | 21, 35, 28 |
| PPCS | phosphopantothenoylcysteine synthetase | 127125 | 066185 | 186409 | 4, 3, 23 |
| PEA15 | phosphoprotein enriched in astrocytes 15 | 162734 | 143318 | 132716 | 8, 15, 27 |
| PGBD2 | piggyBac transposable element derived 2 | 185220 | 171163 | 064703 | 29, 34, 18 |
| PGBD5 | piggyBac transposable element derived 5 | 177614 | 143641 | 135775 | 31, 20, 8 |
| PITHD1 | PITH domain containing 1 | 057757 | 011007 | 011009 | 29, 3, 26 |
| PKP1 | plakophilin 1 | 081277 | 163395 | 118194 | 24, 35, 18 |
| PAFAH2 | platelet activating factor acetylhydrolase 2 | 158006 | 117632 | 158008 | 21, 36, 11 |
| PTAFR | platelet activating factor receptor | 169403 | 158161 | 126698 | 5, 30, 21 |
| PEAR1 | platelet endothelial aggregation receptor 1 | 187800 | 027644 | 160838 | 43, 26, 38 |
| PLEKHG5 | pleckstrin homology and RhoGEF domain containing G5 | 171680 | 215788 | 162408 | 24, 18, 8 |
| PLEKHM2 | pleckstrin homology and RUN domain containing M2 | 116786 | 215695 | 162461 | 34, 14, 35 |
| PLEKHA6 | pleckstrin homology domain containing A6 | 143850 | 174567 | 158615 | |
| PLEKHN1 | pleckstrin homology domain containing N1 | 187583 | 187961 | 187642 | 40, 23, 12 |
| PLEKHO1 | pleckstrin homology domain containing O1 | 023902 | 136631 | 143401 | 41, 5, 6 |
| PHLDA3 | pleckstrin homology like domain family A member 3 | 174307 | 159173 | 159176 | 35, 4, 9 |
| PLXNA2 | plexin A2 | 076356 | 174059 | 008118 | 25, 40, 15 |
| PMF1-BGLAP | PMF1-BGLAP readthrough | 260238 | 160785 | 160783 | |
| PODN | podocan | 174348 | 279054 | 162383 | 39, 42, 1 |
| PDPN | podoplanin | 162493 | 162494 | 116731 | 24, 42, 10 |
| POGK | pogo transposable element with KRAB domain | 143157 | 188859 | 152382 | 11, 42, 18 |
| POGZ | pogo transposable element with ZNF domain | 143442 | 159377 | 143375 | 31, 1, 22 |
| PLK3 | polo like kinase 3 | 173846 | 142959 | 188396 | 27, 1, 25 |
| PABPC4 | poly(A) binding protein cytoplasmic 4 | 090621 | 183682 | 163909 | 9, 35, 16 |
| PARP1 | poly(ADP-ribose) polymerase 1 | 143799 | 183814 | 203685 | 21, 22, 12 |
| PMF1 | polyamine modulated factor 1 | 160783 | 260238 | 242252 | 29, 20, 41 |
| PHC2 | polyhomeotic homolog 2 | 134686 | 184389 | 121903 | 34, 18, 35 |
| PIGR | polymeric immunoglobulin receptor | 162896 | 162894 | 162897 | 16, 37, 25 |
| GALNT2 | polypeptide N-acetylgalactosaminyltransferase 2 | 143641 | 135763 | 177614 | 39, 24, 42 |
| PTBP2 | polypyrimidine tract binding protein 2 | 117569 | 122481 | 188641 | 38, 26, 8 |
| KCNN3 | potassium calcium-activated channel subfamily N member 3 | 143603 | 160710 | 163344 | 22, 21, 25 |
| KCTD3 | potassium channel tetramerization domain containing 3 | 136636 | 082482 | 042781 | 18, 4, 23 |
| KCNT2 | potassium sodium-activated channel subfamily T member 2 | 162687 | 162630 | 000971 | 31, 42, 17 |
| KCNK1 | potassium two pore domain channel subfamily K member 1 | 135750 | 143674 | 183780 | 3, 11, 2 |
| KCNK2 | potassium two pore domain channel subfamily K member 2 | 082482 | 117724 | 136636 | 2, 40, 35 |
| KCNA10 | potassium voltage-gated channel subfamily A member 10 | 143105 | 143125 | 177301 | |
| KCNA2 | potassium voltage-gated channel subfamily A member 2 | 177301 | 143105 | 177272 | |
| KCNA3 | potassium voltage-gated channel subfamily A member 3 | 177272 | 177301 | 143119 | 39, 30, 28 |
| KCNAB2 | potassium voltage-gated channel subfamily A regulatory beta subunit 2 | 069424 | 131697 | 116254 | 7, 8, 39 |
| KCNC4 | potassium voltage-gated channel subfamily C member 4 | 116396 | 197106 | 162775 | 43, 29, 28 |
| KCND3 | potassium voltage-gated channel subfamily D member 3 | 171385 | 064703 | 143079 | 8, 20, |
| KCNH1 | potassium voltage-gated channel subfamily H member 1 | 143473 | 054392 | 117625 | 31, 42, 2 |
| KCNJ10 | potassium voltage-gated channel subfamily J member 10 | 177807 | 162723 | 143315 | 8, 11, 26 |
| KCNJ9 | potassium voltage-gated channel subfamily J member 9 | 162728 | 143315 | 162729 | 8, 10, 38 |
| KCNQ4 | potassium voltage-gated channel subfamily Q member 4 | 117013 | 066136 | 179862 | 5, 14, 37 |
| POU2F1 | POU class 2 homeobox 1 | 143190 | 198842 | 198821 | 21, 39, 24 |
| POU3F1 | POU class 3 homeobox 1 | 185668 | 183520 | 116954 | 11, 35, 20 |
| PERM1 | PPARGC1 and ESRR induced regulator, muscle 1 | 187642 | 187583 | 188290 | 25, 24, 3 |
| PQLC2 | PQ loop repeat containing 2 | 040487 | 053371 | 077549 | 36, 2, 4 |
| PRDM16 | PR/SET domain 16 | 142611 | 169717 | 130762 | 11, 13, 42 |
| PRDM2 | PR/SET domain 2 | 116731 | 162493 | 189337 | 22, 6, 14 |
| PRAMEF1 | PRAME family member 1 | 116721 | 116726 | 239810 | |
| PRAMEF10 | PRAME family member 10 | 187545 | 243073 | 204510 | |
| PRAMEF11 | PRAME family member 11 | 239810 | 116721 | 179172 | |
| PRAMEF12 | PRAME family member 12 | 116726 | 157330 | 116721 | |
| PRAMEF14 | PRAME family member 14 | 204481 | 204501 | 204480 | |
| PRAMEF15 | PRAME family member 15 | 204501 | 182330 | 204481 | |
| PRAMEF17 | PRAME family member 17 | 204479 | 204480 | 204478 | |
| PRAMEF18 | PRAME family member 18 | 279804 | 204505 | 270601 | |
| PRAMEF19 | PRAME family member 19 | 204480 | 204481 | 204479 | |
| PRAMEF2 | PRAME family member 2 | 120952 | 179172 | 243073 | 38, |
| PRAMEF20 | PRAME family member 20 | 204478 | 204479 | 162494 | |
| PRAMEF25 | PRAME family member 25 | 229571 | 274764 | 277058 | |
| PRAMEF26 | PRAME family member 26 | 280267 | 275774 | 179412 | |
| PRAMEF27 | PRAME family member 27 | 274764 | 232423 | 229571 | |
| PRAMEF4 | PRAME family member 4 | 243073 | 120952 | 187545 | |
| PRAMEF5 | PRAME family member 5 | 270601 | 279804 | 182330 | |
| PRAMEF6 | PRAME family member 6 | 232423 | 204510 | 274764 | |
| PRAMEF7 | PRAME family member 7 | 204510 | 187545 | 232423 | |
| PRAMEF8 | PRAME family member 8 | 182330 | 270601 | 204501 | |
| PRAMEF9 | PRAME family member 9 | 204505 | 179412 | 279804 | |
| PFDN2 | prefoldin subunit 2 | 143256 | 162755 | 158793 | 15, 3, 29 |
| PRPF3 | pre-mRNA processing factor 3 | 117360 | 278939 | 163125 | 21, 18, 6 |
| PRPF38A | pre-mRNA processing factor 38A | 134748 | 085840 | 134744 | 30, 7, 39 |
| PRPF38B | pre-mRNA processing factor 38B | 134186 | 162639 | 143107 | 13, 36, 25 |
| PSEN2 | presenilin 2 | 143801 | 143772 | 163050 | 36, 33, 29 |
| PIFO | primary cilia formation | 173947 | 134216 | 085465 | 29, 42, 30 |
| PLOD1 | procollagen-lysine,2-oxoglutarate 5-dioxygenase 1 | 083444 | 116685 | 116688 | 45, 35, 43 |
| PAQR6 | progestin and adipoQ receptor family member 6 | 160781 | 242252 | 198952 | 8, 17, 3 |
| PAQR7 | progestin and adipoQ receptor family member 7 | 182749 | 127423 | 117632 | 40, 32, 38 |
| PROK1 | prokineticin 1 | 143125 | 134248 | 143105 | 27, 38, 32 |
| PRELP | proline and arginine rich end leucine rich repeat protein | 188783 | 122176 | 188770 | 10, 2, 14 |
| PSRC1 | proline and serine rich coiled-coil 1 | 134222 | 143126 | 221986 | 35, 9, 25 |
| PRR9 | proline rich 9 | 203783 | 203784 | 203782 | 15, 12, |
| PRRC2C | proline rich coiled-coil 2C | 117523 | 076258 | 283683 | 18, 27, 24 |
| PNRC2 | proline rich nuclear receptor coactivator 2 | 189266 | 188822 | 188529 | |
| P3H1 | prolyl 3-hydroxylase 1 | 117385 | 164007 | 164008 | 6, 18, 35 |
| PARS2 | prolyl-tRNA synthetase 2, mitochondrial (putative) | 162396 | 243725 | 006555 | 13, 36, 25 |
| PCSK9 | proprotein convertase subtilisin/kexin type 9 | 169174 | 162399 | 162402 | 24, 9, 3 |
| PROX1 | prospero homeobox 1 | 117707 | 136643 | 143499 | 14, 19, 16 |
| PTGER3 | prostaglandin E receptor 3 | 050628 | 116761 | 132485 | 17, 18, 25 |
| PTGFR | prostaglandin F receptor | 122420 | 137960 | 137959 | 10, 42, 6 |
| PTGFRN | prostaglandin F2 receptor inhibitor | 134247 | 116824 | 134256 | 37, 15, 24 |
| PTGS2 | prostaglandin-endoperoxide synthase 2 | 073756 | 116703 | 116711 | 45, 5, 9 |
| PRSS38 | protease, serine 38 | 185888 | 081692 | 143816 | 38, |
| PSMD4 | proteasome 26S subunit, non-ATPase 4 | 159352 | 143398 | 143373 | 3, 34, 12 |
| PSMA5 | proteasome subunit alpha 5 | 143106 | 134243 | 143028 | 29, 1, 35 |
| PSMB2 | proteasome subunit beta 2 | 126067 | 116819 | 142686 | 36, 39, 13 |
| PSMB4 | proteasome subunit beta 4 | 159377 | 143416 | 143442 | 21, 35, 9 |
| PRMT6 | protein arginine methyltransferase 6 | 198890 | 187733 | 162631 | 4, 45, 12 |
| PIAS3 | protein inhibitor of activated STAT 3 | 131788 | 186364 | 198483 | 24, 39, 1 |
| PRKAA2 | protein kinase AMP-activated catalytic subunit alpha 2 | 162409 | 162407 | 187889 | 25, 15, 14 |
| PRKAB2 | protein kinase AMP-activated non-catalytic subunit beta 2 | 131791 | 268043 | 131781 | 11, 42, 6 |
| PRKCZ | protein kinase C zeta | 067606 | 187730 | 162585 | 32, 8, 21 |
| PRKACB | protein kinase cAMP-activated catalytic subunit beta | 142875 | 137941 | 203943 | 42, 8, 39 |
| PKN2 | protein kinase N2 | 065243 | 143013 | 137947 | 4, 42, 43 |
| POMGNT1 | protein O-linked mannose N-acetylglucosaminyltransferase 1 (beta 1,2-) | 085998 | 250719 | 171357 | 40, 33, 31 |
| PPP1R12B | protein phosphatase 1 regulatory subunit 12B | 077157 | 077152 | 143858 | 15, 25, 26 |
| PPP1R15B | protein phosphatase 1 regulatory subunit 15B | 158615 | 143850 | 133056 | 13, 42, 5 |
| PPP1R8 | protein phosphatase 1 regulatory subunit 8 | 117751 | 117758 | 278868 | 3, 21, 31 |
| PPP2R5A | protein phosphatase 2 regulatory subunit B′alpha | 066027 | 143476 | 065600 | |
| PPM1J | protein phosphatase, Mg2+/Mn2+dependent 1J | 155367 | 271810 | 184599 | 41, 30, 34 |
| PTP4A2 | protein tyrosine phosphatase type IVA, member 2 | 184007 | 134668 | 121774 | 1, 6, 5 |
| PTPN14 | protein tyrosine phosphatase, non-receptor type 14 | 152104 | 143499 | 117724 | |
| PTPN22 | protein tyrosine phosphatase, non-receptor type 22 | 134242 | 081019 | 188761 | 39, 22, 10 |
| PTPN7 | protein tyrosine phosphatase, non-receptor type 7 | 143851 | 143862 | 133067 | 39, 36, 5 |
| PTPRC | protein tyrosine phosphatase, receptor type C | 081237 | 151418 | 116833 | 30, 10, 5 |
| PTPRF | protein tyrosine phosphatase, receptor type F | 142949 | 178922 | 066135 | 43, 4, 26 |
| PTPRU | protein tyrosine phosphatase, receptor type U | 060656 | 116353 | 162510 | 14, 12, 3 |
| PRG4 | proteoglycan 4 | 116690 | 143341 | 047410 | 10, 1, 9 |
| PPOX | protoporphyrinogen oxidase | 143224 | 143258 | 158850 | 2, 39, 5 |
| PRUNE1 | prune exopolyphosphatase | 143363 | 143409 | 163141 | 29, 24, 40 |
| PUSH | pseudouridylate synthase-like 1 | 169972 | 131584 | 127054 | 35, 13, 32 |
| PINK1 | PTEN induced putative kinase 1 | 158828 | 158825 | 244038 | 26, 38, 34 |
| PPFIA4 | PTPRF interacting protein alpha 4 | 143847 | 163444 | 122180 | |
| PUM1 | pumilio RNA binding family member 1 | 134644 | 162512 | 084628 | 42, 24, 43 |
| PCP4L1 | Purkinje cell protein 4 like 1 | 248485 | 143257 | 158887 | 31, 4, 14 |
| PHTF1 | putative homeodomain transcription factor 1 | 116793 | 081026 | 081019 | 38, 22, 26 |
| PYGO2 | pygopus family PHD finger 2 | 163348 | 163346 | 160691 | 4, 9, 22 |
| PYHIN1 | pyrin and HIN domain family member 1 | 163564 | 163563 | 163565 | 5, 22, 44 |
| PYCR2 | pyrroline-5-carboxylate reductase family member 2 | 143811 | 255835 | 143768 | 9, 6, 5 |
| PKLR | pyruvate kinase, liver and RBC | 143627 | 143630 | 160752 | 19, 17, 35 |
| QSOX1 | quiescin sulfhydryl oxidase 1 | 116260 | 135837 | 121454 | 20, 38, 32 |
| RABGGTB | Rab geranylgeranyltransferase beta subunit | 137955 | 117054 | 057468 | 11, 6, 34 |
| RABGAP1L | RAB GTPase activating protein 1 like | 152061 | 135870 | 203737 | |
| RABIF | RAB interacting factor | 183155 | 117139 | 117153 | 23, 31, 38 |
| RAB13 | RAB13, member RAS oncogene family | 143545 | 143543 | 177954 | 43, 26, 11 |
| RAB25 | RAB25, member RAS oncogene family | 132698 | 116586 | 254726 | 30, 27, 37 |
| RAB29 | RAB29, member RAS oncogene family | 117280 | 069275 | 133065 | |
| RAB3GAP2 | RAB3 GTPase activating non-catalytic protein subunit 2 | 118873 | 067704 | 116141 | 11, 22, 2 |
| RAB3B | RAB3B, member RAS oncogene family | 169213 | 078618 | 117862 | 28, 11, 31 |
| RAB42 | RAB42, member RAS oncogene family | 188060 | 120656 | 162419 | 34, 17, 35 |
| RAB4A | RAB4A, member RAS oncogene family | 168118 | 177800 | 213029 | 37, 43, 33 |
| RAB7B | RAB7B, member RAS oncogene family | 276600 | 174502 | 196188 | 24, 39, 15 |
| RAD54L | RAD54-like (S. cerevisiae) | 085999 | 171357 | 132128 | 39, 37, 27 |
| RALGPS2 | Ral GEF with PH domain and SH3 binding motif 2 | 116191 | 240021 | 116194 | 22, 38, 14 |
| RGL1 | ral guanine nucleotide dissociation stimulator like 1 | 143344 | 162704 | 173627 | 1, 34, 29 |
| RAP1GAP | RAP1 GTPase activating protein | 076864 | 162551 | 090686 | 3, 40, 31 |
| RAP1A | RAP1 A, member of RAS oncogene family | 116473 | 282608 | 197852 | 11, 45, 29 |
| RORC | RAR related orphan receptor C | 143365 | 213171 | 225556 | 39, 31, 42 |
| RASSF5 | Ras association domain family member 5 | 266094 | 263528 | 143486 | 39, 36, 42 |
| RHOC | ras homolog family member C | 155366 | 155363 | 271810 | 34, 29, 35 |
| RHOU | ras homolog family member U | 116574 | 168159 | 177800 | 29, 6, 2 |
| RIT1 | Ras like without CAAX1 | 143622 | 132718 | 132680 | 40, 11, 13 |
| RASAL2 | RAS protein activator like 2 | 075391 | 120341 | 188585 | 26, 25, 14 |
| RRAGC | Ras related GTP binding C | 116954 | 185668 | 214114 | 28, 6, 1 |
| RBBP4 | RB binding protein 4, chromatin remodeling factor | 162521 | 176261 | 162520 | 4, 45, 7 |
| RBBP5 | RB binding protein 5, histone lysine methyltransferase complex subunit | 117222 | 174529 | 133059 | 22, 45, 43 |
| RCAN3 | RCAN family member 3 | 117602 | 001461 | 184454 | |
| RCSD1 | RCSD domain containing 1 | 198771 | 143162 | 197965 | 41, 22, 21 |
| RLF | rearranged L-myc fusion | 117000 | 131238 | 188800 | 1, 24, 4 |
| ROR1 | receptor tyrosine kinase like orphan receptor 1 | 185483 | 079739 | 177414 | 1, 26, 11 |
| REG4 | regenerating family member 4 | 134193 | 134240 | 134249 | 37, 3, 16 |
| RIMS3 | regulating synaptic membrane exocytosis 3 | 117016 | 117010 | 066136 | 21, 26, 8 |
| RPRD2 | regulation of nuclear pre-mRNA domain containing 2 | 163125 | 117360 | 143374 | 22, 7, 24 |
| RCC1 | regulator of chromosome condensation 1 | 180198 | 204138 | 180098 | 41, 3, 21 |
| RCC2 | regulator of chromosome condensation 2 | 179051 | 276747 | 074964 | |
| RGS1 | regulator of G-protein signaling 1 | 090104 | 253148 | 127074 | 42, 39, 4 |
| RGS13 | regulator of G-protein signaling 13 | 127074 | 090104 | 116741 | 41, 21, 30 |
| RGS16 | regulator of G-protein signaling 16 | 143333 | 135828 | 135824 | 40, 36, 17 |
| RGS18 | regulator of G-protein signaling 18 | 150681 | 162670 | 253148 | 36, 5, 7 |
| RGS2 | regulator of G-protein signaling 2 | 116741 | 127074 | 116750 | 5, 7, 14 |
| RGS21 | regulator of G-protein signaling 21 | 253148 | 150681 | 090104 | |
| RGS4 | regulator of G-protein signaling 4 | 117152 | 185860 | 143248 | 45, 17, 8 |
| RGS5 | regulator of G-protein signaling 5 | 143248 | 117152 | 143228 | 45, 2, 26 |
| RGS7 | regulator of G-protein signaling 7 | 182901 | 180875 | 091483 | 26, 31, 10 |
| RGS8 | regulator of G-protein signaling 8 | 135824 | 143333 | 135838 | 10, 38, 8 |
| RGSL1 | regulator of G-protein signaling like 1 | 121446 | 203730 | 135828 | 38, 40, 33 |
| RSC1A1 | regulator of solute carriers 1 | 215695 | 197312 | 116786 | 26, 11, 40 |
| RFX5 | regulatory factor X5 | 143390 | 143393 | 143416 | 42, 21, 39 |
| RIIAD1 | regulatory subunit of type IIPKA R-subunit (RIIa) domain containing 1 | 178796 | 159409 | 143436 | 31, 38, 14 |
| RXFP4 | relaxin/insulin like family peptide receptor 4 | 173080 | 132680 | 116584 | |
| RSG1 | REM2 and RAB like small GTPase 1 | 132881 | 142632 | 037637 | 5, 15, 35 |
| REN | renin | 143839 | 143845 | 170498 | 16, 17, 4 |
| RPTN | repetin | 215853 | 159450 | 197915 | 24, 35, 23 |
| RPA2 | replication protein A2 | 117748 | 130775 | 130768 | 30, 21, 29 |
| RCOR3 | REST corepressor 3 | 117625 | 143473 | 082512 | 11, 42, 40 |
| RER1 | retention in endoplasmic reticulum sorting receptor 1 | 157916 | 116151 | 157911 | 29, 22, 13 |
| RD3 | retinal degeneration 3 | 198570 | 082512 | 170385 | 14, 38, 12 |
| RXRG | retinoid X receptor gamma | 143171 | 162761 | 162763 | 40, 14, 31 |
| RBP7 | retinol binding protein 7 | 162444 | 173614 | 130939 | 1, 10, 28 |
| RHCE | Rh blood group CcEe antigens | 188672 | 183726 | 204178 | 10, 5, 7 |
| RHD | Rh blood group D antigen | 187010 | 117616 | 183726 | 31, 5, 7 |
| RHBG | Rh family B glycoprotein (gene/pseudogene) | 132677 | 163467 | 125462 | 19, 27, 17 |
| ARHGAP29 | Rho GTPase activating protein 29 | 137962 | 198691 | 117528 | 45, 17, 19 |
| ARHGAP30 | Rho GTPase activating protein 30 | 186517 | 158773 | 278883 | 36, 39, 5 |
| ARHGEF10L | Rho guanine nucleotide exchange factor 10 like | 074964 | 179051 | 117148 | 42, 10, 3 |
| ARHGEF11 | Rho guanine nucleotide exchange factor 11 | 132694 | 160838 | 253831 | 30, 26, 18 |
| ARHGEF16 | Rho guanine nucleotide exchange factor 16 | 130762 | 142611 | 162591 | 37, 26, 9 |
| ARHGEF19 | Rho guanine nucleotide exchange factor 19 | 142632 | 142627 | 132881 | 3, 9, 27 |
| ARHGEF2 | Rho/Rac guanine nucleotide exchange factor 2 | 116584 | 173080 | 163479 | |
| RHBDL2 | rhomboid like 2 | 158315 | 131233 | 174574 | 18, 2, 20 |
| RNASEL | ribonuclease L | 135828 | 121446 | 143333 | 29, 22, 26 |
| RAVER2 | ribonucleoprotein, PTB binding 2 | 162437 | 158966 | 162434 | 42, 33, 11 |
| RIMKLA | ribosomal modification protein rimK like family member A | 177181 | 198815 | 066185 | 14, 24, 20 |
| RPL11 | ribosomal protein L11 | 142676 | 197880 | 011007 | 43, 12, 1 |
| RPL22 | ribosomal protein L22 | 116251 | 116254 | 158286 | 29, 15, 1 |
| RPL5 | ribosomal protein L5 | 122406 | 067208 | 154511 | 41, 43, 21 |
| RPS27 | ribosomal protein S27 | 177954 | 143545 | 143552 | |
| RPS6KA1 | ribosomal protein S6 kinase A1 | 117676 | 198830 | 117713 | 36, 21, 39 |
| RPS6KC1 | ribosomal protein S6 kinase C1 | 136643 | 174606 | 117707 | 6, 26, 42 |
| RPS8 | ribosomal protein S8 | 142937 | 142945 | 142959 | 41, 43, 21 |
| RRNAD1 | ribosomal RNA adenine dimethylase domain containing 1 | 143303 | 143319 | 143314 | |
| RRP15 | ribosomal RNA processing 15 homolog | 067533 | 162814 | 092969 | 34, 10, 4 |
| RPF1 | ribosome production factor 1 homolog | 117133 | 137976 | 174021 | 34, 30, 43 |
| RC3H1 | ring finger and CCCH-type domains 1 | 135870 | 117601 | 152061 | 25, 22, 24 |
| RFWD2 | ring finger and WD repeat domain 2 | 143207 | 116147 | 116183 | 35, 37, 4 |
| RNF11 | ring finger protein 11 | 123091 | 204006 | 085831 | 29, 11, 1 |
| RNF115 | ring finger protein 115 | 265491 | 117281 | 186141 | 41, 15, 18 |
| RNF186 | ring finger protein 186 | 178828 | 162542 | 169914 | 16, 17, 27 |
| RNF187 | ring finger protein 187 | 168159 | 196890 | 116574 | 34, 21, 27 |
| RNF19B | ring finger protein 19B | 116514 | 121900 | 004455 | 13, 38, 27 |
| RNF2 | ring finger protein 2 | 121481 | 135842 | 121486 | 13, 45, 5 |
| RNF207 | ring finger protein 207 | 158286 | 116251 | 116237 | 29, 15, 40 |
| RNF220 | ring finger protein 220 | 187147 | 117419 | 126106 | |
| RNF223 | ring finger protein 223 | 237330 | 188157 | 131591 | 9, |
| RTCA | RNA 3′-terminal phosphate cyclase | 137996 | 137992 | 079335 | 34, 1, 43 |
| RBM15 | RNA binding motif protein 15 | 162775 | 116396 | 168679 | 38, 22, 19 |
| RBM34 | RNA binding motif protein 34 | 188739 | 173726 | 054267 | 30, 7, 21 |
| RBM8A | RNA binding motif protein 8A | 265241 | 131779 | 280778 | 42, 24, 39 |
| RBMXL1 | RNA binding motif protein, X-linked like 1 | 213516 | 137944 | 117226 | 29, 36, 42 |
| RNPC3 | RNA binding region (RNP1, RRM) containing 3 | 185946 | 060718 | 240038 | 31, 1, 40 |
| RPAP2 | RNA polymerase II associated protein 2 | 122484 | 174842 | 162676 | 29, 30, 9 |
| POLR3C | RNApolymerase III subunit C | 186141 | 265491 | 186364 | 5, 23, 37 |
| POLR3GL | RNApolymerase III subunit G like | 121851 | 272031 | 265972 | 41, 11, 31 |
| RSBN1 | round spermatid basic protein 1 | 081019 | 116793 | 134242 | 22, 38, 7 |
| RPE65 | RPE65, retinoid isomerohydrolase | 116745 | 116729 | 024526 | 14, 8, |
| RSP01 | R-spondin 1 | 169218 | 134697 | 116922 | 29, 2, 15 |
| RUSC1 | RUN and SH3 domain containing 1 | 160753 | 160752 | 116539 | 9, 43, 13 |
| RUNX3 | runt related transcription factor 3 | 020633 | 169504 | 117614 | 22, 5, 36 |
| RWDD3 | RWD domain containing 3 | 122481 | 271092 | 117569 | 4, 39, 28 |
| RYR2 | ryanodine receptor 2 | 198626 | 244020 | 116996 | 15, 9, 19 |
| S100A1 | S100 calcium binding protein A1 | 160678 | 189171 | 160679 | 15, 25, 35 |
| S100A10 | S100 calcium binding protein A10 | 197747 | 159445 | 163191 | 23, 15, 11 |
| S100A11 | S100 calcium binding protein A11 | 163191 | 197747 | 182898 | 4, 18, 23 |
| S100A12 | S100 calcium binding protein A12 | 163221 | 163220 | 143546 | 7, 5, 1 |
| S100A13 | S100 calcium binding protein A13 | 189171 | 189334 | 160678 | 29, 15, 31 |
| S100A14 | S100 calcium binding protein A14 | 189334 | 188643 | 189171 | 40, 37, 13 |
| S100A16 | S100 calcium binding protein A16 | 188643 | 196754 | 189334 | 30, 35, 6 |
| S100A2 | S100 calcium binding protein A2 | 196754 | 188015 | 188643 | 4, 18, 30 |
| S100A3 | S100 calcium binding protein A3 | 188015 | 196154 | 196754 | 15, 18, 6 |
| S100A4 | S100 calcium binding protein A4 | 196154 | 196420 | 188015 | 3, 1, 19 |
| S100A5 | S100 calcium binding protein A5 | 196420 | 197956 | 196154 | 10, 22, |
| S100A6 | S100 calcium binding protein A6 | 197956 | 143556 | 196420 | 43, 37, 3 |
| S100A7 | S100 calcium binding protein A7 | 143556 | 197364 | 197956 | 18, 30, 24 |
| S100A7L2 | S100 calcium binding protein A7 like 2 | 197364 | 184330 | 143556 | |
| S100A7A | S100 calcium binding protein A7A | 184330 | 143546 | 197364 | 30, 18, 24 |
| S100A8 | S100 calcium binding protein A8 | 143546 | 163221 | 184330 | 30, 24, 7 |
| S100A9 | S100 calcium binding protein A9 | 163220 | 163218 | 163221 | 7, 18, 24 |
| S100PBP | SWOP binding protein | 116497 | 134684 | 160097 | 22, 4, 39 |
| SCCPDH | saccharopine dehydrogenase (putative) | 143653 | 162852 | 153207 | 38, 31, 29 |
| SASS6 | SAS-6 centriolar assembly protein | 156876 | 156875 | 122435 | 4, 39, 19 |
| SLFNL1 | schlafen like 1 | 171790 | 171793 | 010803 | 38, 42, 8 |
| STIL | SCL/TAL1 interrupting locus | 123473 | 162367 | 162368 | |
| SCYL3 | SCY1 like pseudokinase 3 | 000457 | 171806 | 075945 | 13, 24, 1 |
| SDE2 | SDE2 telomere maintenance homolog | 143751 | 143768 | 163041 | 34, 9, 4 |
| SEC16B | SEC16 homolog B, endoplasmic reticulum export factor | 120341 | 198797 | 075391 | 19, 26, 42 |
| SEC22B | SEC22 homolog B, vesicle trafficking protein (gene/pseudogene) | 265808 | 134250 | 273136 | 39, 20, 38 |
| SCAMP3 | secretory carrier membrane protein 3 | 116521 | 160767 | 176444 | 9, 16, 23 |
| SZT2 | seizure threshold 2 homolog (mouse) | 198198 | 159479 | 178922 | 18, 4, 33 |
| SELE | selectin E | 007908 | 188404 | 171806 | 42, 29, 44 |
| SELL | selectin L | 188404 | 000460 | 007908 | 5, 36, 30 |
| SELP | selectin P | 174175 | 198734 | 000460 | 42, 10, 18 |
| SELENBP1 | selenium binding protein 1 | 143416 | 143390 | 159377 | 16, 1, 36 |
| SELENOF | selenoprotein F | 183291 | 097033 | 153936 | 30, 29, 7 |
| SELENON | selenoprotein N | 162430 | 117643 | 255054 | 43, 35, 27 |
| SEMA4A | semaphorin 4A | 196189 | 160789 | 160785 | 24, 39, 40 |
| SEMA6C | semaphorin 6C | 143434 | 143458 | 163154 | |
| SRRM1 | serine and arginine repetitive matrix 1 | 133226 | 184454 | 169504 | 43, 22, 3 |
| SRSF10 | serine and arginine rich splicing factor 10 | 188529 | 189266 | 142661 | 30, 22, 29 |
| SRSF11 | serine and arginine rich splicing factor 11 | 116754 | 066557 | 118454 | 4, 12, 2 |
| SRSF4 | serine and arginine rich splicing factor 4 | 116350 | 253304 | 116353 | 21, 13, 11 |
| SERINC2 | serine incorporator 2 | 168528 | 121769 | 142910 | 23, 4, 32 |
| STK40 | serine/threonine kinase 40 | 196182 | 142694 | 181817 | 37, 22, 21 |
| SDCCAG8 | serologically defined colon cancer antigen 8 | 054282 | 143702 | 117020 | 11, 10, 40 |
| SERPINC1 | serpin family C member 1 | 117601 | 185278 | 135870 | 19, 36, 23 |
| SERBP1 | SERPINE1 mRNA binding protein 1 | 142864 | 081985 | 116717 | 11, 25, 29 |
| SERTAD4 | SERTA domain containing 4 | 082497 | 283619 | 054392 | 11, 37, 32 |
| SARS | seryl-tRNA synthetase | 031698 | 116299 | 143126 | 43, 21, 9 |
| SESN2 | sestrin 2 | 130766 | 130770 | 130772 | 21, 34, 14 |
| SMYD2 | SET and MYND domain containing 2 | 143499 | 117707 | 152104 | 10, 31, 22 |
| SMYD3 | SET and MYND domain containing 3 | 185420 | 162849 | 162851 | 10, 34, 26 |
| SETDB1 | SET domain bifurcated 1 | 143379 | 283324 | 143418 | 41, 1, 24 |
| SETSIP | SET-like protein | 230667 | 172031 | 189195 | |
| SCMH1 | sex comb on midleg homolog 1 (Drosophila) | 010803 | 171790 | 204060 | 26, 39, 38 |
| SFT2D2 | SFT2 domain containing 2 | 213064 | 143155 | 143178 | 39, 36, 38 |
| SH2D1B | SH2 domain containing 1B | 198574 | 171722 | 152332 | 36, 5, 30 |
| SH2D2A | SH2 domain containing 2A | 027869 | 143294 | 198400 | 5, 39, 37 |
| SH2D5 | SH2 domain containing 5 | 189410 | 117245 | 127483 | 22, 32, 3 |
| SH3BP5L | SH3 binding domain protein 5 like | 175137 | 259823 | 171161 | 39, 24, 38 |
| SH3BGRL3 | SH3 domain binding glutamate rich protein like 3 | 142669 | 130695 | 158062 | 30, 3, 43 |
| SH3D21 | SH3 domain containing 21 | 214193 | 054118 | 142694 | 37, 4, 30 |
| SH3GLB1 | SH3 domain containing GRB2 like endophilin B1 | 097033 | 016602 | 183291 | 1, 42, 38 |
| SGIP1 | SH3 domain GRB2 like endophilin interacting protein 1 | 118473 | 184588 | 152760 | |
| SHC1 | SHC adaptor protein 1 | 160691 | 163348 | 173207 | 24, 39, 42 |
| SHCBP1L | SHC binding and spindle associated 1 like | 157060 | 135829 | 135862 | 38, 8, |
| SHISA4 | shisa family member 4 | 198892 | 198700 | 163431 | 25, 2, 8 |
| SIPA1L2 | signal induced proliferation associated 1 like 2 | 116991 | 162946 | 212916 | 34, 43, 26 |
| SRP9 | signal recognition particle 9 | 143742 | 154380 | 143819 | |
| SSR2 | signal sequence receptor subunit 2 | 163479 | 116584 | 160803 | 13, 43, 27 |
| SLAMF1 | signaling lymphocytic activation molecule family member 1 | 117090 | 066294 | 117091 | 39, 22, 30 |
| SSBP3 | single stranded DNA binding protein 3 | 157216 | 116221 | 162390 | |
| SKI | SKI proto-oncogene | 157933 | 162585 | 116151 | 35, 22, 28 |
| SLAMF6 | SLAM family member 6 | 162739 | 162738 | 066294 | 36, 22, 5 |
| SLAMF7 | SLAM family member 7 | 026751 | 117091 | 122224 | 36, 5, 13 |
| SLAMF8 | SLAM family member 8 | 158714 | 181036 | 188004 | 1, 31, 20 |
| SLAMF9 | SLAM family member 9 | 162723 | 085552 | 177807 | 6, 5, 38 |
| SRGAP2 | SLIT-ROBO Rho GTPase activating protein 2 | 266028 | 196550 | 263528 | |
| SRGAP2B | SLIT-ROBO Rho GTPase activating protein 2B | 196369 | 279782 | 215784 | |
| SRGAP2C | SLIT-ROBO Rho GTPase activating protein 2C | 171943 | 188610 | 203818 | 11, 26, 14 |
| SNIP1 | Smad nuclear interacting protein 1 | 163877 | 163875 | 163879 | 43, 31, 36 |
| SMAP2 | small ArfGAP2 | 084070 | 049089 | 187801 | 41, 5, 39 |
| SMIM1 | small integral membrane protein 1 (Vel blood group) | 235169 | 162592 | 130764 | 17, 38, 7 |
| SMIM12 | small integral membrane protein 12 | 163866 | 142698 | 189280 | 8, |
| SNRPE | small nuclear ribonucleoprotein polypeptide E | 182004 | 257315 | 143842 | |
| SNRNP40 | small nuclear ribonucleoprotein U5 subunit 40 | 060688 | 084628 | 121766 | 21, 31, 12 |
| SPRR1A | small proline rich protein 1A | 169474 | 184148 | 163209 | 30, 18, 24 |
| SPRR1B | small proline rich protein 1B | 169469 | 163209 | 163216 | 30, 18, 24 |
| SPRR2A | small proline rich protein 2A | 241794 | 163216 | 196805 | 30, 24, 18 |
| SPRR2B | small proline rich protein 2B | 196805 | 241794 | 203785 | 30, 13, 10 |
| SPRR2D | small proline rich protein 2D | 163216 | 169469 | 241794 | 30, 10, 13 |
| SPRR2E | small proline rich protein 2E | 203785 | 196805 | 244094 | 30, 35, 18 |
| SPRR2F | small proline rich protein 2F | 244094 | 203785 | 159516 | 30, 27, 10 |
| SPRR2G | small proline rich protein 2G | 159516 | 244094 | 203784 | 30, 10, 24 |
| SPRR3 | small proline rich protein 3 | 163209 | 169474 | 169469 | 30, 24, 13 |
| SPRR4 | small proline rich protein 4 | 184148 | 283227 | 169474 | 30, 15, 33 |
| SVBP | small vasohibin binding protein | 177868 | 274386 | 164010 | |
| SMG5 | SMG5, nonsense mediated mRNA decay factor | 198952 | 160781 | 163472 | 33, 21, 12 |
| SMG7 | SMG7, nonsense mediated mRNA decay factor | 116698 | 157064 | 116701 | 22, 40, 9 |
| SNAPIN | SNAP associated protein | 143553 | 160679 | 143621 | 21, 41, 6 |
| SCNN1D | sodium channel epithelial 1 delta subunit | 162572 | 160087 | 131584 | 38, 28, 27 |
| SCNM1 | sodium channel modifier 1 | 163156 | 163154 | 163155 | 3, 6, 30 |
| NKAIN1 | Sodium/potassium transporting ATPase interacting 1 | 084628 | 134644 | 060688 | 26, 14, 4 |
| SLC1A7 | solute carrier family 1 member 7 | 162383 | 174348 | 157184 | 14, 42, 25 |
| SLC16A1 | solute carrier family 16 member 1 | 155380 | 184599 | 198799 | 38, 13, 45 |
| SLC16A4 | solute carrier family 16 member 4 | 168679 | 162775 | 134248 | 32, 45, 11 |
| SLC19A2 | solute carrier family 19 member 2 | 117479 | 117477 | 198734 | 1, 11, 45 |
| SLC2A1 | solute carrier family 2 member 1 | 117394 | 164011 | 186973 | 24, 13, 32 |
| SLC2A5 | solute carrier family 2 member 5 | 142583 | 197241 | 180758 | 38, 22, 21 |
| SLC2A7 | solute carrier family 2 member 7 | 197241 | 131686 | 142583 | 45, |
| SLC22A15 | solute carrier family 22 member 15 | 163393 | 177551 | 173212 | 26, 20, 4 |
| SLC25A24 | solute carrier family 25 member 24 | 085491 | 134215 | 196427 | 45, 42, 7 |
| SLC25A33 | solute carrier family 25 member 33 | 171612 | 171621 | 188807 | 3, 15, 17 |
| SLC25A34 | solute carrier family 25 member 34 | 162461 | 116786 | 162460 | 1, 25, 4 |
| SLC25A44 | solute carrier family 25 member 44 | 160785 | 196189 | 260238 | 8, 24, 29 |
| SLC26A9 | solute carrier family 26 member 9 | 174502 | 162877 | 276600 | 28, 30, 15 |
| SLC27A3 | solute carrier family 27 member 3 | 143554 | 279767 | 143614 | 1, 35, 5 |
| SLC30A1 | solute carrier family 30 member 1 | 170385 | 198570 | 117650 | 29, 26, 45 |
| SLC30A10 | solute carrier family 30 member 10 | 196660 | 215817 | 136628 | 38, 19, 8 |
| SLC30A2 | solute carrier family 30 member 2 | 158014 | 158008 | 158022 | 32, 17, 40 |
| SLC30A7 | solute carrier family 30 member 7 | 162695 | 162694 | 117543 | 45, 42, 32 |
| SLC35A3 | solute carrier family 35 member A3 | 117620 | 283495 | 156875 | 18, 45, 17 |
| SLC35D1 | solute carrier family 35 member D1 | 116704 | 198160 | 203963 | 39, 13, 6 |
| SLC35E2 | solute carrier family 35 member E2 | 215790 | 008128 | 008130 | |
| SLC35E2B | solute carrier family 35 member E2B | 189339 | 248333 | 008128 | 11, 31, 16 |
| SLC35F3 | solute carrier family 35 member F3 | 183780 | 135750 | 168275 | 29, 20, 28 |
| SLC39A1 | solute carrier family 39 member 1 | 143570 | 160741 | 143578 | 9, 13, 32 |
| SLC41A1 | solute carrier family 41 member 1 | 133065 | 117280 | 162877 | 40, 22, 33 |
| SLC44A3 | solute carrier family 44 member 3 | 143036 | 117525 | 117519 | 31, 37, 16 |
| SLC44A5 | solute carrier family 44 member 5 | 137968 | 162624 | 117054 | 24, 11, 38 |
| SLC45A1 | solute carrier family 45 member 1 | 162426 | 116285 | 142599 | 31, 14, 27 |
| SLC45A3 | solute carrier family 45 member 3 | 158715 | 158711 | 069275 | 33, 26, 36 |
| SLC5A9 | solute carrier family 5 member 9 | 117834 | 279096 | 132122 | 17, 38, 16 |
| SLC50A1 | solute carrier family 50 member 1 | 169241 | 169242 | 179085 | 34, 41, 28 |
| SLC6A17 | solute carrier family 6 member 17 | 197106 | 186150 | 116396 | 26, 31, 14 |
| SLC6A9 | solute carrier family 6 member 9 | 196517 | 159214 | 171872 | 44, 20, |
| SLC9A1 | solute carrier family 9 member A1 | 090020 | 280403 | 142784 | 40, 43, 42 |
| SLC9C2 | solute carrier family 9 member C2 (putative) | 162753 | 117592 | 183831 | 38, 27, 10 |
| SORT1 | sortilin 1 | 134243 | 221986 | 143106 | 29, 8, 26 |
| SNX7 | sorting nexin 7 | 162627 | 188641 | 117598 | 10, 4, 44 |
| SNX27 | sorting nexin family member 27 | 143376 | 143367 | 159409 | 1, 2, 16 |
| SPTA1 | spectrin alpha, erythrocytic 1 | 163554 | 198967 | 196171 | 7, 1, 19 |
| SPEN | spen family transcriptional repressor | 065526 | 233954 | 116809 | 22, 26, 6 |
| SPAG17 | sperm associated antigen 17 | 155761 | 065183 | 092607 | 42, 38, 20 |
| SMCP | sperm mitochondria associated cysteine rich protein | 163206 | 235942 | 163207 | 38, 26, 32 |
| STPG1 | sperm tail PG-rich repeat containing 1 | 001460 | 158055 | 001461 | |
| SPATA17 | spermatogenesis associated 17 | 162814 | 092978 | 067533 | 10, 29, 38 |
| SPATA21 | spermatogenesis associated 21 | 187144 | 055070 | 157191 | 38, 19, |
| SPATA45 | spermatogenesis associated 45 | 185523 | 203705 | 162769 | |
| SPATA6 | spermatogenesis associated 6 | 132122 | 117834 | 186094 | 38, 11, 12 |
| SRM | spermidine synthase | 116649 | 009724 | 171824 | 35, 34, 21 |
| SPHAR | S-phase response (cyclin related) | 213029 | 168118 | 154429 | |
| SMPDL3B | sphingomyelin phosphodiesterase acid like 3B | 130768 | 117748 | 158156 | 29, 4, 16 |
| S1PR1 | sphingosine-1-phosphate receptor 1 | 170989 | 117543 | 118733 | 45, 1, 22 |
| SPSB1 | splA/ryanodine receptor domain and SOCS box containing 1 | 171621 | 049239 | 171612 | 43, 35, 28 |
| SF3A3 | splicing factor 3a subunit 3 | 183431 | 204084 | 183386 | 21, 30, 31 |
| SF3B4 | splicing factor 3b subunit 4 | 143368 | 159164 | 014914 | 41, 21, 5 |
| SFPQ | splicing factor proline and glutamine rich | 116560 | 197056 | 146463 | 23, 44, 19 |
| SPOCD1 | SPOC domain containing 1 | 134668 | 121753 | 184007 | 34, 45, 38 |
| SPRTN | SprT-like N-terminal domain | 010072 | 116903 | 135766 | 38, 29, 42 |
| SHE | Src homology 2 domain containing E | 169291 | 160712 | 163239 | 1, 38, 40 |
| SOX13 | SRY-box 13 | 143842 | 182004 | 143845 | 35, 18, 42 |
| SSU72 | SSU72 homolog, RNA polymerase IICTD phosphatase | 160075 | 205090 | 279244 | 27, 22, 21 |
| SSX2IP | SSX family member 2 interacting protein | 117155 | 117151 | 171517 | 38, 6, 18 |
| ST3GAL3 | ST3 beta-galactoside alpha-2,3-sialyltransferase 3 | 126091 | 066135 | 117407 | 18, 21, 31 |
| ST6GALNAC3 | ST6 N-acetylgalactosaminide alpha-2,6-sialyltransferase 3 | 184005 | 154007 | 117069 | |
| ST6GALNAC5 | ST6 N-acetylgalactosaminide alpha-2,6-sialyltransferase 5 | 117069 | 184005 | 142892 | 4, 26, 8 |
| STMN1 | stathmin 1 | 117632 | 182749 | 158006 | 34, 39, 4 |
| SAMD11 | sterile alpha motif domain containing 11 | 187634 | 273547 | 188976 | 34, 14, 28 |
| SAMD13 | sterile alpha motif domain containing 13 | 203943 | 142875 | 137976 | 34, 31, 22 |
| SCP2 | sterol carrier protein 2 | 116171 | 121310 | 279054 | 4, 7, 34 |
| SOAT1 | sterol O-acyltransferase 1 | 057252 | 143322 | 162779 | 2, 42, 4 |
| SFN | stratifin | 175793 | 204160 | 142751 | 13, 18, 35 |
| STRIP1 | striatin interacting protein 1 | 143093 | 168710 | 156150 | 38, 24, 39 |
| SDF4 | stromal cell derived factor 4 | 078808 | 186827 | 176022 | 6, 34, 3 |
| STUM | stum, mechanosensory transduction mediator homolog | 203685 | 143799 | 143772 | 26, 8, 25 |
| SDHB | succinate dehydrogenase complex iron sulfur subunit B | 117118 | 159363 | 117115 | 41, 29, 35 |
| SDHC | succinate dehydrogenase complex subunit C | 143252 | 158887 | 188931 | 23, 27, 17 |
| SUCO | SUN domain containing ossification factor | 094975 | 180999 | 117560 | 11, 4, 28 |
| ST7L | suppression of tumorigenicity 7 like | 007341 | 134245 | 116489 | 38, 10, 5 |
| SIKE1 | suppressor of IKBKE1 | 052723 | 009307 | 198765 | 22, 26, 45 |
| SUSD4 | sushi domain containing 4 | 143502 | 187554 | 178395 | 24, 8, 28 |
| SZRD1 | SUZ RNA binding domain containing 1 | 055070 | 037637 | 187144 | 42, 24, 1 |
| SWT1 | SWT1, RNA endoribonuclease homolog | 116668 | 121486 | 116679 | 38, 25, 26 |
| SYF2 | SYF2 pre-mRNA splicing factor | 117614 | 020633 | 117616 | 2, 6, 5 |
| SYDE2 | synapse defective Rho GTPase homolog 2 | 097096 | 055732 | 162642 | 18, 40, 27 |
| SV2A | synaptic vesicle glycoprotein 2A | 159164 | 178096 | 143368 | 8, 34, 39 |
| SYCP1 | synaptonemal complex protein 1 | 198765 | 052723 | 134200 | 10, 38, |
| SYPL2 | synaptophysin like 2 | 143028 | 143106 | 162650 | 15, 2, 25 |
| SNAP47 | synaptosome associated protein 47 | 143740 | 181450 | 081692 | 9, 27, 14 |
| SYT11 | synaptotagmin 11 | 132718 | 116580 | 143622 | 8, 31, 26 |
| SYT14 | synaptotagmin 14 | 143469 | 117597 | 283619 | 21, 6, 38 |
| SYT2 | synaptotagmin 2 | 143858 | 077157 | 117139 | 18, 8, 2 |
| SYT6 | synaptotagmin 6 | 134207 | 116774 | 197323 | 28, 12, 8 |
| SYTL1 | synaptotagmin like 1 | 142765 | 186501 | 142733 | 39, 36, 13 |
| SYNC | syncoilin, intermediate filament protein | 162520 | 162521 | 162522 | 11, 45, 6 |
| SDC3 | syndecan 3 | 162512 | 162511 | 134644 | 43, 35, 26 |
| STX12 | syntaxin 12 | 117758 | 009780 | 117751 | 7, 29, 40 |
| STX6 | syntaxin 6 | 135823 | 135835 | 153029 | 11, 4, 39 |
| STXBP3 | syntaxin binding protein 3 | 116266 | 143107 | 162641 | 31, 4, 39 |
| TAL1 | TAL bHLH transcription factor 1, erythroid differentiation factor | 162367 | 162366 | 123473 | |
| TARBP1 | TAR (HIV-1) RNA binding protein 1 | 059588 | 168275 | 168264 | 2, 37, 44 |
| TARDBP | TAR DNA binding protein | 120948 | 175262 | 277726 | 39, 13, 45 |
| TOE1 | target of EGR1, member 1 (nuclear) | 132773 | 132781 | 070759 | 9, 40, 29 |
| TAS1R1 | taste 1 receptor member 1 | 173662 | 162408 | 204859 | 36, 6, 38 |
| TAS1R2 | taste 1 receptor member 2 | 179002 | 009709 | 255275 | |
| TAS1R3 | taste 1 receptor member 3 | 169962 | 224051 | 107404 | 31, 25, 33 |
| TAF12 | TATA-box binding protein associated factor 12 | 120656 | 180098 | 188060 | 4, 7, 10 |
| TAF13 | TATA-box binding protein associated factor 13 | 197780 | 085433 | 215717 | 23, 2, 30 |
| TAF5L | TATA-box binding protein associated factor 5 like | 135801 | 135776 | 135763 | 23, 11, 24 |
| TAF1A | TATA-box binding protein associated factor, RNA polymerase I subunit A | 143498 | 143512 | 154305 | 30, 22, 24 |
| TATDN3 | TatD DNase domain containing 3 | 203705 | 117697 | 185523 | 34, 7, 1 |
| TXLNA | taxilin alpha | 084652 | 025800 | 160050 | 18, 43, 35 |
| TBX15 | T-box 15 | 092607 | 155761 | 116874 | 26, 11, 10 |
| TBX19 | T-box 19 | 143178 | 213064 | 143185 | 31, 20, 2 |
| TCTEX1D1 | Tctex1 domain containing 1 | 152760 | 118473 | 172410 | 45, 20, 8 |
| TCTEX1D4 | Tctex1 domain containing 4 | 188396 | 173846 | 222009 | 20, 32, |
| TEKT2 | tektin 2 | 092850 | 126070 | 116863 | 42, 38, 30 |
| TNN | tenascin N | 120332 | 120333 | 235750 | 42, 14, 5 |
| TNR | tenascin R | 116147 | 235750 | 143207 | 8, 23, 19 |
| TEX35 | testis expressed 35 | 240021 | 188585 | 116191 | 38, 32, 25 |
| TEX38 | testis expressed 38 | 186118 | 123472 | 159658 | 38, 5, 8 |
| TSSK3 | testis specific serine kinase 3 | 162526 | 175130 | 225828 | 25, 12, |
| TESK2 | testis-specific kinase 2 | 070759 | 132773 | 280670 | 39, 38, 13 |
| TSPAN1 | tetraspanin 1 | 117472 | 278139 | 250719 | 30, 16, 33 |
| TSPAN2 | tetraspanin 2 | 134198 | 134200 | 134259 | 29, 44, 17 |
| TTC13 | tetratricopeptide repeat domain 13 | 143643 | 119280 | 173409 | 22, 28, 37 |
| TTC22 | tetratricopeptide repeat domain 22 | 006555 | 162396 | 162398 | 29, 13, 9 |
| TTC24 | tetratricopeptide repeat domain 24 | 187862 | 183856 | 163382 | 41, 39, 36 |
| TTC34 | tetratricopeptide repeat domain 34 | 215912 | 142606 | 169717 | |
| TTC39A | tetratricopeptide repeat domain 39A | 085831 | 123091 | 085832 | 38, 23, 16 |
| TTC4 | tetratricopeptide repeat domain 4 | 243725 | 271723 | 162396 | 43, 9, 13 |
| THAP3 | THAP domain containing 3 | 041988 | 116273 | 007923 | 21, 26, 38 |
| THEM4 | thioesterase superfamily member 4 | 159445 | 196407 | 197747 | 3, 31, 13 |
| THEM5 | thioesterase superfamily member 5 | 196407 | 225556 | 159445 | 35, 18, 24 |
| TXNDC12 | thioredoxin domain containing 12 | 117862 | 169213 | 198841 | 29, 6, 37 |
| TXNIP | thioredoxin interacting protein | 265972 | 121851 | 168509 | 39, 36, 42 |
| TSTD1 | thiosulfate sulfurtransferase like domain containing 1 | 215845 | 270149 | 158773 | 30, 23, 21 |
| TARS2 | threonyl-tRNA synthetase 2, mitochondrial (putative) | 143374 | 163125 | 143369 | 9, 23, 24 |
| THBS3 | thrombospondin 3 | 169231 | 279487 | 173171 | 37, 10, 38 |
| THEMIS2 | thymocyte selection associated family member 2 | 130775 | 278868 | 117748 | 36, 5, 7 |
| THRAP3 | thyroid hormone receptor associated protein 3 | 054118 | 116871 | 214193 | 30, 18, 7 |
| TSHB | thyroid stimulating hormone beta | 134200 | 198765 | 134198 | 31, 28, 10 |
| TM2D1 | TM2 domain containing 1 | 162604 | 162599 | 132849 | 1, 38, 40 |
| TMEM56- | TMEM56-RWDD3 readthrough | 271092 | 152078 | 122481 | |
| RWDD3 | | | | | |
| TRNP1 | TMF1-regulated nuclear protein 1 | 253368 | 175707 | 158246 | 37, 28, 33 |
| TNFAIP8L2 | TNF alpha induced protein 8 like 2 | 163154 | 143434 | 163156 | 30, 39, 2 |
| TRAF5 | TNF receptor associated factor 5 | 082512 | 117625 | 198570 | 29, 22, 9 |
| TNFRSF14 | TNF receptor superfamily member 14 | 157873 | 197921 | 157870 | 35, 34, 29 |
| TNFRSF18 | TNF receptor superfamily member 18 | 186891 | 162571 | 186827 | 13, 22, 37 |
| TNFRSF1B | TNF receptor superfamily member 1B | 028137 | 120949 | 048707 | 36, 28, 1 |
| TNFRSF25 | TNF receptor superfamily member 25 | 215788 | 187017 | 171680 | 3, 36, 9 |
| TNFRSF4 | TNF receptor superfamily member 4 | 186827 | 186891 | 078808 | 37, 5, 22 |
| TNFRSF8 | TNF receptor superfamily member 8 | 120949 | 116691 | 028137 | 34, 5, 44 |
| TNFRSF9 | TNF receptor superfamily member 9 | 049249 | 049247 | 116288 | 30, 5, 20 |
| TNNI3K | TNNI3 interacting kinase | 116783 | 259030 | 162621 | 10, 42, 15 |
| TLR5 | toll like receptor 5 | 187554 | 154309 | 143502 | 24, 23, 44 |
| TIPRL | TOR signaling pathway regulator | 143155 | 143147 | 213064 | 34, 18, 9 |
| TOR1AIP1 | torsin 1A interacting protein 1 | 143337 | 169905 | 135837 | 42, 4, 39 |
| TOR1AIP2 | torsin 1A interacting protein 2 | 169905 | 143340 | 143337 | |
| TOR3A | torsin family 3 member A | 186283 | 116199 | 143322 | 41, 21, 9 |
| TRABD2B | TraB domain containing 2B | 269113 | 186564 | 279096 | 17, 15, 44 |
| TRAF3IP3 | TRAF3 interacting protein 3 | 009790 | 117594 | 162757 | 39, 36, 5 |
| TRAPPC3 | trafficking protein particle complex 3 | 054116 | 171812 | 116871 | 18, 1, 34 |
| TCEANC2 | transcription elongation factor A N-terminal and central domain containing 2 | 116205 | 116209 | 256407 | 26, 37, 41 |
| TCEA3 | transcription elongation factor A3 | 204219 | 125945 | 088280 | 25, 26, 15 |
| TCEB3 | Transcription elongation factor B subunit 3 | 011007 | 142676 | 057757 | 3, 22, 41 |
| TFAP2E | transcription factor AP-2 epsilon | 116819 | 020129 | 126067 | 44, 8, |
| TFB2M | transcription factor B2, mitochondrial | 162851 | 185420 | 162852 | 3, 10, 7 |
| TTF2 | transcription termination factor 2 | 116830 | 134256 | 134253 | 23, 12, 1 |
| TADA1 | transcriptional adaptor 1 | 152382 | 143157 | 143195 | 7, 29, 9 |
| TGFB2 | transforming growth factor beta 2 | 092969 | 067533 | 143353 | 11, 23, 10 |
| TGFBR3 | transforming growth factor beta receptor 3 | 069702 | 097046 | 137948 | 11, 1, 31 |
| TAGLN2 | transgelin 2 | 158710 | 213085 | 085552 | 1, 41, 21 |
| TSNAX | translin associated factor X | 116918 | 135766 | 162946 | |
| TIMM17A | translocase of inner mitochondrial membrane 17 homolog A (yeast) | 134375 | 163431 | 176393 | 34, 7, 29 |
| TOMM20 | translocase of outer mitochondrial membrane 20 | 173726 | 279261 | 188739 | 42, 24, 8 |
| TOMM40L | translocase of outer mitochondrial membrane 40 like | 158882 | 158874 | 143257 | 34, 19, 35 |
| TPR | translocated promoter region, nuclear basket protein | 047410 | 116690 | 157181 | 18, 7, 37 |
| TMCC2 | transmembrane and coiled-coil domain family 2 | 133069 | 133059 | 163545 | 8, 42, 14 |
| TMCO1 | transmembrane and coiled-coil domains 1 | 143183 | 143149 | 143179 | |
| TMCO2 | transmembrane and coiled-coil domains 2 | 188800 | 117000 | 084073 | 38, 10, 32 |
| TMCO4 | transmembrane and coiled-coil domains 4 | 162542 | 158748 | 178828 | |
| TMIGD3 | transmembrane and immunoglobulin domain containing 3 | 121933 | 143110 | 282608 | |
| TEDDM1 | transmembrane epididymal protein 1 | 203730 | 135821 | 121446 | 38, |
| TMED5 | transmembrane p24 trafficking protein 5 | 117500 | 143033 | 122483 | 22, 42, 37 |
| TMEM125 | transmembrane protein 125 | 179178 | 243710 | 253313 | 16, 31, 2 |
| TMEM167B | transmembrane protein 167B | 215717 | 197780 | 179902 | 29, 24, 1 |
| TMEM183A | transmembrane protein 183A | 163444 | 159348 | 143847 | 34, 12, 22 |
| TMEM200B | transmembrane protein 200B | 253304 | 159023 | 116350 | 11, 15, 37 |
| TMEM201 | transmembrane protein 201 | 188807 | 171612 | 171608 | 28, 26, 27 |
| TMEM206 | transmembrane protein 206 | 065600 | 066027 | 117691 | 18, 22, 39 |
| TMEM222 | transmembrane protein 222 | 186501 | 142784 | 142765 | 34, 26, 11 |
| TMEM234 | transmembrane protein 234 | 160055 | 222046 | 084623 | 39, 36, 38 |
| TMEM240 | transmembrane protein 240 | 205090 | 197785 | 160075 | 33, 32, |
| TMEM35B | transmembrane protein 35B | 243749 | 271741 | 163867 | |
| TMEM39B | transmembrane protein 39B | 121775 | 121774 | 025800 | 43, 6, 3 |
| TMEM50A | transmembrane protein 50A | 183726 | 187010 | 188672 | 29, 2, 36 |
| TMEM51 | transmembrane protein 51 | 171729 | 189337 | 142621 | 29, 18, 37 |
| TMEM52 | transmembrane protein 52 | 178821 | 169885 | 142609 | 25, 3, 38 |
| TMEM53 | transmembrane protein 53 | 126106 | 187147 | 198520 | 30, 6, 38 |
| TMEM54 | transmembrane protein 54 | 121900 | 121905 | 116514 | 16, 32, 37 |
| TMEM56 | transmembrane protein 56 | 152078 | 172339 | 271092 | 4, 9, 42 |
| TMEM57 | transmembrane protein 57 | 204178 | 188672 | 157978 | 26, 3, 14 |
| TMEM59 | transmembrane protein 59 | 116209 | 203985 | 116205 | 13, 29, 31 |
| TMEM61 | transmembrane protein 61 | 143001 | 116133 | 162399 | 28, 31, 17 |
| TMEM63A | transmembrane protein 63A | 196187 | 143819 | 243709 | 16, 42, 1 |
| TMEM69 | transmembrane protein 69 | 159596 | 159592 | 197429 | 22, 34, 10 |
| TMEM78 | transmembrane protein 78 | 177800 | 116574 | 168118 | 38, |
| TMEM79 | transmembrane protein 79 | 163472 | 198952 | 198715 | 33, 24, 13 |
| TMEM81 | transmembrane protein 81 | 174529 | 184144 | 117222 | 29, 26, 28 |
| TMEM82 | transmembrane protein 82 | 162460 | 162461 | 162458 | 19, |
| TMEM88B | transmembrane protein 88B | 205116 | 235098 | 179403 | |
| TMEM9 | transmembrane protein 9 | 116857 | 232237 | 163395 | 9, 27, 14 |
| TCHH | trichohyalin | 159450 | 182898 | 215853 | 30, 32, 15 |
| TCHHL1 | trichohyalin like 1 | 182898 | 163191 | 159450 | |
| TRIM11 | tripartite motif containing 11 | 154370 | 154358 | 162931 | 1, 43, 21 |
| TRIM17 | tripartite motif containing 17 | 162931 | 154370 | 168148 | 36, 31, 38 |
| TRIM33 | tripartite motif containing 33 | 197323 | 134207 | 116752 | |
| TRIM45 | tripartite motif containing 45 | 134253 | 116830 | 134258 | |
| TRIM46 | tripartite motif containing 46 | 163462 | 273088 | 185499 | 8, 39, 31 |
| TRIM58 | tripartite motif containing 58 | 162722 | 197591 | 238243 | 34, 26, 18 |
| TRIM62 | tripartite motif containing 62 | 116525 | 142920 | 160094 | 26, 22, 32 |
| TRIM63 | tripartite motif containing 63 | 158022 | 158014 | 175087 | 15, 25, 35 |
| TRIM67 | tripartite motif containing 67 | 119283 | 182118 | 143633 | 8, 14, 35 |
| TRIT1 | tRNA isopentenyltransferase 1 | 043514 | 198754 | 116990 | 7, 4, 44 |
| TRMT1L | tRNA methyltransferase 1 like | 121486 | 121481 | 116668 | 42, 38, 36 |
| TRMT13 | tRNA methyltransferase 13 homolog | 122435 | 156876 | 122477 | 22, 26, 7 |
| TRNAU1AP | tRNA selenocysteine 1 associated protein 1 | 180098 | 180198 | 120656 | 21, 30, 11 |
| TSEN15 | tRNA splicing endonuclease subunit 15 | 198860 | 198756 | 116667 | 6, 43, 9 |
| TYW3 | tRNA-yW synthesizing protein 3 homolog | 162623 | 116791 | 162624 | 26, 31, 42 |
| TMOD4 | tropomodulin 4 | 163157 | 163155 | 163159 | 25, 15, 18 |
| TPM3 | tropomyosin 3 | 143549 | 143552 | 163263 | |
| TNNI1 | troponin 11, slow skeletal type | 159173 | 159166 | 174307 | 25, 15, 24 |
| TNNT2 | troponin T2, cardiac type | 118194 | 081277 | 159166 | 15, 18, 2 |
| TROVE2 | TROVE domain family member 2 | 116747 | 116750 | 023572 | 30, 11, 5 |
| WARS2 | tryptophanyl tRNA synthetase 2, mitochondrial | 116874 | 092607 | 116882 | 9, 45, 24 |
| TSNAX- | TSNAX-DISC1 readthrough (NMD candidate) | 270106 | 135766 | 162946 | |
| DISC1 | | | | | |
| TSACC | TSSK6 activating cochaperone | 163467 | 163468 | 132677 | 38, 10, 33 |
| TBCE | tubulin folding cofactor E | 116957 | 152904 | 162885 | 2, 1, 3 |
| TTLL10 | tubulin tyrosine ligase like 10 | 162571 | 131591 | 186891 | 38, 30, 25 |
| TTLL7 | tubulin tyrosine ligase like 7 | 137941 | 117114 | 142875 | 10, 38, 42 |
| TINAGL1 | tubulointerstitial nephritis antigen like 1 | 142910 | 168528 | 121764 | 32, 45, 1 |
| TDRKH | tudorand KH domain containing | 182134 | 143450 | 213171 | 38, 6, 8 |
| TDRD10 | tudor domain containing 10 | 163239 | 169291 | 160714 | 26, 38, 44 |
| TDRD5 | tudor domain containing 5 | 162782 | 116218 | 143340 | 38, 18, 20 |
| TUFT1 | tuftelin 1 | 143367 | 143375 | 143376 | 24, 18, 26 |
| TNFSF18 | tumor necrosis factor superfamily member 18 | 120337 | 117560 | 117586 | 45, 25, 8 |
| TNFSF4 | tumor necrosis factor superfamily member 4 | 117586 | 120337 | 117592 | 18, 7, 10 |
| TP53BP2 | tumor protein p53 binding protein 2 | 143514 | 162909 | 143756 | 8, 29, 5 |
| TPRG1L | tumor protein p63 regulated 1 like | 158109 | 162591 | 116213 | 15, 37, 35 |
| TP73 | tumor protein p73 | 078900 | 116213 | 162592 | 24, 42, 22 |
| TACSTD2 | tumor-associated calcium signal transducer 2 | 184292 | 162600 | 162601 | 27, 18, 30 |
| TIE1 | tyrosine kinase with immunoglobulin like and EGF like domains 1 | 066056 | 253313 | 117400 | 43, 45, 32 |
| YARS | tyrosyl-tRNA synthetase | 134684 | 162522 | 116497 | 21, 36, 3 |
| UHMK1 | U2AF homology motif kinase 1 | 152332 | 198574 | 117143 | 37, 22, 42 |
| UBIAD1 | UbiA prenyltransferase domain containing 1 | 120942 | 171819 | 204624 | 3, 14, 34 |
| UBQLN4 | ubiquilin 4 | 160803 | 163479 | 116586 | 21, 34, 9 |
| UQCRH | ubiquinol-cytochrome c reductase hinge protein | 173660 | 132128 | 117481 | 29, 3, 19 |
| UQCRHL | ubiquinol-cytochrome c reductase hinge protein like | 233954 | 162458 | 065526 | 29, 15, 14 |
| UBAP2L | ubiquitin associated protein 2 like | 143569 | 143612 | 143575 | |
| UBE2J2 | ubiquitin conjugating enzyme E2 J2 | 160087 | 184163 | 162572 | 27, 37, 41 |
| UBE2Q1 | ubiquitin conjugating enzyme E2 Q1 | 160714 | 163239 | 160716 | 14, 27, 6 |
| UBE2T | ubiquitin conjugating enzyme E2 T | 077152 | 133067 | 077157 | 34, 7, 4 |
| UBE2U | ubiquitin conjugating enzyme E2 U (putative) | 177414 | 185483 | 158966 | 38, 4, 32 |
| UCHL5 | ubiquitin C-terminal hydrolase L5 | 116750 | 116741 | 116747 | 14, 4, 7 |
| UBL4B | ubiquitin like 4B | 186150 | 156150 | 197106 | 38, 14, 8 |
| UBR4 | ubiquitin protein ligase E3 component n-recognin 4 | 127481 | 169991 | 127463 | 18, 43, 6 |
| USP1 | ubiquitin specific peptidase 1 | 162607 | 132854 | 116641 | 38, 4, 42 |
| USP21 | ubiquitin specific peptidase 21 | 143258 | 143222 | 143224 | 3, 30, 38 |
| USP24 | ubiquitin specific peptidase 24 | 162402 | 169174 | 162407 | 22, 6, 23 |
| USP33 | ubiquitin specific peptidase 33 | 077254 | 036549 | 180488 | 42, 11, 22 |
| USP48 | ubiquitin specific peptidase 48 | 090686 | 076864 | 187942 | 2, 10, 32 |
| UBE4B | ubiquitination factor E4B | 130939 | 162444 | 054523 | 29, 22, 11 |
| UFC1 | ubiquitin-fold modifier conjugating enzyme 1 | 143222 | 158796 | 143258 | 29, 30, 4 |
| UBXN10 | UBX domain protein 10 | 162543 | 187980 | 158816 | 42, 38, 23 |
| UBXN11 | UBX domain protein 11 | 158062 | 142669 | 169442 | 36, 39, 38 |
| GALE | UDP-galactose-4-epimerase | 117308 | 011009 | 117305 | 16, 35, 37 |
| UAP1 | UDP-N-acetylglucosamine pyrophosphorylase 1 | 117143 | 152332 | 162733 | 24, 10, 1 |
| USF1 | upstream transcription factor 1 | 158773 | 215845 | 186517 | 40, 5, 23 |
| URB2 | URB2 ribosome biogenesis 2 homolog (S. cerevisiae) | 135763 | 135801 | 143641 | 18, 21, 31 |
| UCK2 | uridine-cytidine kinase 2 | 143179 | 143183 | 188859 | 43, 3, 32 |
| UROD | uroporphyrinogen decarboxylase | 126088 | 126107 | 162415 | 29, 43, 40 |
| UTS2 | urotensin 2 | 049247 | 049246 | 049249 | 36, 7, 16 |
| USH2A | usherin | 042781 | 136636 | 196482 | 10, 14, 38 |
| UTP11 | UTP11, small subunit processome component homolog (S. cerevisiae) | 183520 | 183386 | 185668 | 30, 43, 25 |
| VPS13D | vacuolar protein sorting 13 homolog D | 048707 | 028137 | 162496 | 18, 40, 44 |
| VPS45 | vacuolar protein sorting 45 homolog | 136631 | 264522 | 023902 | 13, 29, 36 |
| VPS72 | vacuolar protein sorting 72 homolog | 163159 | 163157 | 143398 | 9, 23, 6 |
| VANGL1 | VANGL planar cell polarity protein 1 | 173218 | 134259 | 118729 | 2, 6, 15 |
| VANGL2 | VANGL planar cell polarity protein 2 | 162738 | 171786 | 162739 | 12, 14, 18 |
| VCAM1 | vascular cell adhesion molecule 1 | 162692 | 181656 | 162694 | 36, 10, 39 |
| VASH2 | vasohibin 2 | 143494 | 162769 | 174606 | 15, 11, 42 |
| VAV3 | vav guanine nucleotide exchange factor 3 | 134215 | 162631 | 085491 | 42, 24, 32 |
| VAMP3 | vesicle associated membrane protein 3 | 049245 | 171735 | 049246 | 45, 4, 13 |
| VAMP4 | vesicle associated membrane protein 4 | 117533 | 034971 | 010165 | 31, 25, 34 |
| MYCL | v-myc avian myelocytomatosis viral oncogene lung carcinoma derived homolog | 116990 | 043514 | 168389 | |
| VHLL | von Hippel-Lindau tumor suppressor like | 189030 | 198715 | 163468 | |
| VWA1 | von Willebrand factor A domain containing 1 | 179403 | 205116 | 215915 | 28, 33, 16 |
| VWA5B1 | von Willebrand factor A domain containing 5B1 | 158816 | 162543 | 162545 | 38, 8, 19 |
| VSIG8 | V-set and immunoglobulin domain containing 8 | 243284 | 188004 | 256029 | 2, 30, 40 |
| VTCN1 | V-set domain containing T cell activation inhibitor 1 | 134258 | 134253 | 198162 | 24, 27, 42 |
| WASF2 | WAS protein family member 2 | 158195 | 181773 | 126705 | 43, 9, 3 |
| WDTC1 | WD and tetratricopeptide repeats 1 | 142784 | 090020 | 186501 | |
| WRAP73 | WD repeat containing, antisense to TP73 | 116213 | 158109 | 078900 | 21, 1, 5 |
| WDR26 | WD repeat domain 26 | 162923 | 143771 | 143786 | 26, 11, 22 |
| WDR3 | WD repeat domain 3 | 065183 | 196505 | 155761 | 22, 4, 35 |
| WDR47 | WD repeat domain 47 | 085433 | 121940 | 197780 | 26, 8, 42 |
| WDR63 | WD repeat domain 63 | 162643 | 153898 | 055732 | 38, 30, 42 |
| WDR64 | WD repeat domain 64 | 162843 | 203668 | 174371 | 38, 40, 20 |
| WDR77 | WD repeat domain 77 | 116455 | 085465 | 116459 | 13, 41, 45 |
| WDR78 | WD repeat domain 78 | 152763 | 172410 | 198160 | 42, 10, 30 |
| WNT2B | Wnt family member 2B | 134245 | 143079 | 007341 | 38, 14, 32 |
| WNT3A | Wnt family member 3A | 154342 | 143816 | 143761 | 42, 32, 38 |
| WNT4 | Wnt family member 4 | 162552 | 070831 | 184677 | 20, 28, 12 |
| WNT9A | Wnt family member 9A | 143816 | 185888 | 154342 | 18, 20, 9 |
| WLS | wntless Wnt ligand secretion mediator | 116729 | 162595 | 116745 | 45, 42, 38 |
| XCL1 | X-C motif chemokine ligand 1 | 143184 | 143185 | 143196 | 5, 10, 20 |
| XCL2 | X-C motif chemokine ligand 2 | 143185 | 143178 | 143184 | 33, 22, 5 |
| XPR1 | xenotropic and polytropic retrovirus receptor 1 | 143324 | 230124 | 135835 | 13, 19, 11 |
| XKR8 | XK related 8 | 158156 | 130768 | 158161 | 34, 27, 7 |
| YBX1 | Y-box binding protein 1 | 065978 | 171960 | 164007 | 21, 12, 6 |
| YIPF1 | Yip1 domain family member 1 | 058799 | 058804 | 211452 | 31, 41, 5 |
| YOD1 | YOD1 deubiquitinase | 180667 | 123836 | 123843 | |
| YRDC | yrdC N6-threonylcarbamoyltransferase domain containing | 196449 | 185090 | 197982 | 21, 32, 37 |
| YTHDF2 | YTH N6-methyladenosine RNA binding protein 2 | 198492 | 162419 | 116329 | 38, 10, 45 |
| YY1AP1 | YY1 associated protein 1 | 163374 | 125459 | 132676 | 41, 21, 37 |
| ZFP69 | ZFP69 zinc finger protein | 187815 | 187801 | 164002 | 18, 15, 37 |
| ZFP69B | ZFP69 zinc finger protein B | 187801 | 084070 | 187815 | 41, 13, 17 |
| ZBTB17 | zinc finger and BTB domain containing 17 | 116809 | 065526 | 183888 | 36, 39, 1 |
| ZBTB18 | zinc finger and BTB domain containing 18 | 179456 | 117020 | 173728 | 8, 26, 14 |
| ZBTB37 | zinc finger and BTB domain containing 37 | 185278 | 117593 | 117601 | 31, 38, 30 |
| ZBTB40 | zinc finger and BTB domain containing 40 | 184677 | 162552 | 070886 | 24, 22, 29 |
| ZBTB41 | zinc finger and BTB domain containing 41 | 177888 | 066279 | 134376 | 18, 37, 31 |
| ZBTB48 | zinc finger and BTB domain containing 48 | 204859 | 173662 | 162413 | 41, 36, 38 |
| ZBTB7B | zinc finger and BTB domain containing 7B | 160685 | 163352 | 163354 | 42, 3, 33 |
| ZBTB80S | zinc finger and BTB domain containing 8 opposite strand | 176261 | 160062 | 162521 | 10, 1, 3 |
| ZBTB8A | zinc finger and BTB domain containing 8A | 160062 | 273274 | 176261 | 43, 26, 37 |
| ZBTB8B | zinc finger and BTB domain containing 8B | 273274 | 254553 | 160062 | 26, 32, 8 |
| ZSCAN20 | zinc finger and SCAN domain containing 20 | 121903 | 134686 | 121904 | 43, 42, 25 |
| ZBED6 | zinc finger BED-type containing 6 | 257315 | 058673 | 182004 | 15, 3, 5 |
| ZC3H11A | zinc finger CCCH-type containing 11A | 058673 | 122188 | 257315 | 29, 22, 40 |
| ZC3H11B | Zinc finger CCCH-type containing 11B pseudogene | 215817 | 143353 | 196660 | |
| ZC3H12A | zinc finger CCCH-type containing 12A | 163874 | 163873 | 163875 | 30, 36, 13 |
| ZCCHC11 | zinc finger CCHC-type containing 11 | 134744 | 134748 | 116157 | 13, 2, 35 |
| ZCCHC17 | zinc finger CCHC-type containing 17 | 121766 | 060688 | 121769 | 33, 3, 30 |
| ZDHHC18 | zinc finger DHHC-type containing 18 | 204160 | 060642 | 175793 | 5, 20, 34 |
| ZFYVE9 | zinc finger FYVE-type containing 9 | 157077 | 134717 | 154222 | 43, 40, 9 |
| ZNHIT6 | zinc finger HIT-type containing 6 | 117174 | 142871 | 171502 | 11, 9, 10 |
| ZMYM1 | zinc finger MYM-type containing 1 | 197056 | 163867 | 116560 | 30, 39, 29 |
| ZMYM4 | zinc finger MYM-type containing 4 | 146463 | 116560 | 142687 | 11, 1, 40 |
| ZMYM6 | zinc finger MYM-type containing 6 | 163867 | 243749 | 197056 | 31, 22, 30 |
| ZMYND12 | zinc finger MYND-type containing 12 | 066185 | 177181 | 127125 | 38, 30, 27 |
| ZNF124 | zinc finger protein 124 | 196418 | 188295 | 162714 | 24, 23, 14 |
| ZNF281 | zinc finger protein 281 | 162702 | 116833 | 118193 | 24, 12, 6 |
| ZNF326 | zinc finger protein 326 | 162664 | 171492 | 143032 | 22, 1, 6 |
| ZNF362 | zinc finger protein 362 | 160094 | 116525 | 184389 | 15, 23, 18 |
| ZNF436 | zinc finger protein 436 | 125945 | 125944 | 204219 | 1, 4, 24 |
| ZNF496 | zinc finger protein 496 | 162714 | 196418 | 162711 | 18, 39, 9 |
| ZNF593 | zinc finger protein 593 | 142684 | 236782 | 142675 | 41, 21, 32 |
| ZNF644 | zinc finger protein 644 | 122482 | 143032 | 162669 | 22, 7, 3 |
| ZNF648 | zinc finger protein 648 | 179930 | 198216 | 135821 | 42, 35, |
| ZNF669 | zinc finger protein 669 | 188295 | 277462 | 196418 | 18, 4, 45 |
| ZNF670 | zinc finger protein 670 | 277462 | 135747 | 188295 | 38, 4, 39 |
| ZNF672 | zinc finger protein 672 | 171161 | 175137 | 171163 | |
| ZNF678 | zinc finger protein 678 | 181450 | 143776 | 143740 | 16, 22, 36 |
| ZNF683 | zinc finger protein 683 | 176083 | 176092 | 131914 | 5, 38, 6 |
| ZNF684 | zinc finger protein 684 | 117010 | 164002 | 117016 | 7, 11, 31 |
| ZNF687 | zinc finger protein 687 | 143373 | 159352 | 143393 | 41, 43, 14 |
| ZNF691 | zinc finger protein 691 | 164011 | 164010 | 117394 | |
| ZNF692 | zinc finger protein 692 | 171163 | 171161 | 185220 | 24, 38, 36 |
| ZNF695 | zinc finger protein 695 | 197472 | 153207 | 277462 | 4, 7, 12 |
| ZNF697 | zinc finger protein 697 | 143067 | 203857 | 092621 | 35, 26, 2 |
| ZRANB2 | zinc finger RANBP2-type containing 2 | 132485 | 050628 | 172260 | 19, 45, 25 |
| ZSWIM5 | zinc finger SWIM-type containing 5 | 162415 | 126088 | 186603 | |
| ZZZ3 | zinc finger ZZ-type containing 3 | 036549 | 154027 | 077254 | 23, 24, 31 |
| ZMPSTE24 | zinc metallopeptidase STE24 | 084073 | 188800 | 049089 | 4, 42, 13 |
| ZNF670- | ZNF670-ZNF695 readthrough (NMD candidate) | 135747 | 153207 | 277462 | |
| ZNF695 | | | | | |
| ZP4 | zona pellucida glycoprotein 4 | 116996 | 198626 | 270188 | 19, 14, |
| ZYG11A | zyg-11 family member A, cell cycle regulator | 203995 | 162378 | 121310 | 17, 23, 44 |
| ZYG11B | zyg-11 family member B, cell cycle regulator | 162378 | 162377 | 203995 | 11, 25, 19 |

Table 2 provides the gene information for the genes found on Chromosomes 2 and 3.

TABLE 2
Genes on Chromosomes 2 and 3

| | | | | Up | Down | Tissue |
|---|---|---|---|---|---|---|
| Chr. | Gene Symbol | Gene Name | ENSG | ENSG | ENSG | Code |
| 2 | HAAO | 3-hydroxyanthranilate 3,4-dioxygenase | 162882 | 162881 | 152518 | 18, 26, 36 |
| 2 | HIBCH | 3-hydroxyisobutyryl-CoA hydrolase | 198130 | 138379 | 151689 | 17, 18, 31 |
| 2 | ATIC | 5-aminoimidazole-4-carboxamide ribonucleotide formyltransferase/IMP | 138363 | 144452 | 115414 | 21, 7, 3 |
| | | cyclohydrolase | | | | |
| 2 | HTR2B | 5-hydroxytryptamine receptor 2B | 135914 | 173692 | 135931 | 45, 17, 40 |
| 2 | NT5DC4 | 5′-nucleotidase domain containing 4 | 144130 | 144136 | 169607 | 18, 16, 8 |
| 2 | NT5C1B | 5′-nudeotidase, cytosolic IB | 185013 | 250741 | 143867 | 38, |
| 2 | ABHD1 | abhydrolase domain containing 1 | 143994 | 138028 | 138073 | 38, 14, 37 |
| 2 | ABI2 | abI interactor 2 | 138443 | 119004 | 173166 | 8, 31, 43 |
| 2 | ACP1 | acid phosphatase 1, soluble | 143727 | 035115 | 189292 | 29, 31, 32 |
| 2 | ASIC4 | acid sensing ion channel subunit family member 4 | 072182 | 144591 | 123989 | 8, 12, 32 |
| 2 | ADI1 | acireductone dioxygenase 1 | 182551 | 171853 | 255767 | 26, 6, 29 |
| 2 | ARPC2 | actin related protein 2/3 complex subunit 2 | 163466 | 163464 | 179921 | 41, 5, 35 |
| 2 | ACTG2 | actin, gamma 2, smooth muscle, enteric | 163017 | 124356 | 114956 | 43, 16, 33 |
| 2 | ATF2 | activating transcription factor 2 | 115966 | 128656 | 154518 | 25, 5, 8 |
| 2 | AHSA2 | activator of HSP90 ATPase homolog 2 | 173209 | 237651 | 115464 | 8, |
| 2 | ACVR1 | activin A receptor type 1 | 115170 | 123612 | 007001 | 15, 26, 11 |
| 2 | ACVR1C | activin A receptor type 1C | 123612 | 115165 | 115170 | 1, 37, 36 |
| 2 | ACVR2A | activin A receptor type 2A | 121989 | 169554 | 115947 | 29, 43, 24 |
| 2 | ACADL | acyl-CoA dehydrogenase, long chain | 115361 | 144445 | 279317 | 9, 17, 33 |
| 2 | ACOXL | acyl-CoA oxidase-like | 153093 | 169679 | 153094 | 28, 12, 33 |
| 2 | ACSL3 | acyl-CoA synthetase long-chain family member 3 | 123983 | 124003 | 152049 | 29, 33, 42 |
| 2 | ACYP2 | acylphosphatase 2 | 170634 | 068878 | 178021 | 10, 41, 1 |
| 2 | ADAM17 | ADAM metallopeptidase domain 17 | 151694 | 134330 | 134308 | 24, 44, 23 |
| 2 | ADAM23 | ADAM metallopeptidase domain 23 | 114948 | 279921 | 235118 | 13, 11, 29 |
| 2 | AP1S3 | adaptor related protein complex 1 sigma 3 subunit | 152056 | 171951 | 085449 | 41, 22, 21 |
| 2 | ASXL2 | additional sex combs like 2, transcriptional regulator | 143970 | 138101 | 084731 | 31, 24, 11 |
| 2 | ADD2 | adducin 2 | 075340 | 163235 | 183733 | 8, 14, 26 |
| 2 | ADCY3 | adenylate cyclase 3 | 138031 | 138092 | 115137 | 7, 27, 26 |
| 2 | ADGRF3 | adhesion G protein-coupled receptor F3 | 173567 | 138029 | 138018 | |
| 2 | ARL4C | ADP ribosylation factor like GTPase 4C | 188042 | 072080 | 130147 | |
| 2 | ARL5A | ADP ribosylation factor like GTPase 5A | 162980 | 183091 | 283228 | 34, 31, 17 |
| 2 | ARL6IP6 | ADP ribosylation factor like GTPase 6 interacting protein 6 | 177917 | 196504 | 177519 | 22, 26, 10 |
| 2 | ADRA2B | adrenoceptor alpha 2B | 274286 | 186281 | 188886 | 26, 17, 15 |
| 2 | AFF3 | AF4/FMR2 family member 3 | 144218 | 135945 | 170500 | 22, 8, 39 |
| 2 | AFTPH | aftiphilin | 119844 | 119862 | 279644 | 41, 30, 7 |
| 2 | AGXT | alanine-glyoxylate aminotransferase | 172482 | 130294 | 172478 | 19, 5, 17 |
| 2 | AOX1 | aldehyde oxidase 1 | 138356 | 163535 | 280298 | 2, 1, 19 |
| 2 | ALPI | alkaline phosphatase, intestinal | 163295 | 163286 | 171551 | 9, 16, 37 |
| 2 | ALPP | alkaline phosphatase, placental | 163283 | 144535 | 163286 | 32, 13, 15 |
| 2 | ALPPL2 | alkaline phosphatase, placental like 2 | 163286 | 163283 | 163295 | 3, 28, 44 |
| 2 | AGPS | alkylglycerone phosphate synthase | 018510 | 116044 | 196659 | 43, 26, 4 |
| 2 | ALLC | allantoicase | 151360 | 118004 | 214866 | 38, 17, 27 |
| 2 | ATRAID | all-trans retinoic acid induced differentiation factor | 138085 | 138074 | 084774 | 34, 31, 28 |
| 2 | ALMS1 | ALMS1, centrosome and basal body associated protein | 116127 | 135625 | 144035 | 4, 1, 3 |
| 2 | ALS2 | ALS2, alsin Rho guanine nucleotide exchange factor | 003393 | 082126 | 138395 | 13, 35, 22 |
| 2 | ACMSD | aminocarboxymuconate semialdehyde decarboxylase | 153086 | 152128 | 082258 | 17, 11, 19 |
| 2 | AMMECR1L | AMMECR1 like | 144233 | 144231 | 136715 | 40, 9, 37 |
| 2 | ALS2CR11 | amyotrophic lateral sclerosis 2 chromosome region candidate 11 | 155754 | 082146 | 155755 | 38, 26, 7 |
| 2 | ALS2CR12 | amyotrophic lateral sclerosis 2 chromosome region candidate 12 | 155749 | 064012 | 115993 | |
| 2 | ANAPC1 | anaphase promoting complex subunit 1 | 153107 | 153094 | 153208 | 11, 6, 40 |
| 2 | ALK | anaplastic lymphoma receptor tyrosine kinase | 171094 | 115295 | 119801 | 25, 37, 42 |
| 2 | AUP1 | ancient ubiquitous protein 1 | 115307 | 144045 | 115317 | 9, 5, 35 |
| 2 | AAMP | angio associated migratory cell protein | 127837 | 179921 | 127838 | 13, 37, 25 |
| 2 | ANKAR | ankyrin and armadillo repeat containing | 151687 | 138381 | 128694 | 38, 7, 36 |
| 2 | ANKMY1 | ankyrin repeat and MYND domain containing 1 | 144504 | 063660 | 188542 | |
| 2 | ASB1 | ankyrin repeat and SOCS box containing 1 | 065802 | 204104 | 233608 | 40, 18, 22 |
| 2 | ASB18 | ankyrin repeat and SOCS box containing 18 | 182177 | 168505 | 132321 | 24, |
| 2 | ASB3 | ankyrin repeat and SOCS box containing 3 | 115239 | 179915 | 270898 | 41, 13, 26 |
| 2 | ANKZF1 | ankyrin repeat and zinc finger domain containing 1 | 163516 | 198925 | 163521 | 39, 26, 24 |
| 2 | ANKRD23 | ankyrin repeat domain 23 | 163126 | 168763 | 213337 | 25, 7, 1 |
| 2 | ANKRD30BL | ankyrin repeat domain 30B like | 163046 | 163040 | 183840 | 38, 8, |
| 2 | ANKRD36 | ankyrin repeat domain 36 | 135976 | 144199 | 196912 | 19, 18, 1 |
| 2 | ANKRD36B | ankyrin repeat domain 36B | 196912 | 135976 | 135940 | 26, 19, 39 |
| 2 | ANKRD36C | ankyrin repeat domain 36C | 174501 | 144015 | 186281 | 10, 19, 29 |
| 2 | ANKRD39 | ankyrin repeat domain 39 | 213337 | 163126 | 168758 | 22, 6, 14 |
| 2 | ANKRD44 | ankyrin repeat domain 44 | 065413 | 197121 | 283224 | 22, 1, 39 |
| 2 | ANKRD53 | ankyrin repeat domain 53 | 144031 | 258881 | 144043 | 38, 32, 14 |
| 2 | ANXA4 | annexin A4 | 196975 | 115977 | 087338 | 4, 13, 37 |
| 2 | ANO7 | anoctamin 7 | 146205 | 115685 | 115677 | 33, 9, 3 |
| 2 | ANTXR1 | anthrax toxin receptor 1 | 169604 | 169605 | 198380 | 11, 1, 45 |
| 2 | AAK1 | AP2 associated kinase 1 | 115977 | 169599 | 196975 | 29, 40, 4 |
| 2 | AMER3 | APC membrane recruitment protein 3 | 178171 | 173302 | 136002 | 8, 14, 20 |
| 2 | APOB | apolipoprotein B | 084674 | 118961 | 218819 | 19, 36, 25 |
| 2 | APLF | aprataxin and PNKP like factor | 169621 | 204923 | 169618 | |
| 2 | AQP12A | aquaporin 12A | 184945 | 185176 | 130294 | |
| 2 | AQP12B | aquaporin 12B | 185176 | 178623 | 184945 | 28, 16, |
| 2 | AGFG1 | ArfGAP with FG repeats 1 | 173744 | 168955 | 042304 | 11, 4, 29 |
| 2 | AGAP1 | ArfGAP with GTPase domain, ankyrin repeat and PH domain 1 | 157985 | 130147 | 168505 | 26, 14, 23 |
| 2 | ASAP2 | ArfGAP with SH3 domain, ankyrin repeat and PH domain 2 | 151693 | 143797 | 119185 | 37, 24, 18 |
| 2 | RNPEPL1 | arginyl aminopeptidase like 1 | 142327 | 188542 | 142330 | 34, 3, 5 |
| 2 | ARMC9 | armadillo repeat containing 9 | 135931 | 135914 | 156966 | |
| 2 | ACTR1B | ARP1 actin-related protein 1 homolog B, centractin beta | 115073 | 135940 | 228486 | 9, 26, 3 |
| 2 | ACTR2 | ARP2 actin related protein 2 homolog | 138071 | 138069 | 198369 | 22, 25, 6 |
| 2 | ACTR3 | ARP3 actin related protein 3 homolog | 115091 | 115084 | 175497 | 45, 36, 13 |
| 2 | ASNSD1 | asparagine synthetase domain containing 1 | 138381 | 138449 | 151687 | 13, 11, 17 |
| 2 | ASPRV1 | aspartic peptidase, retroviral-like 1 | 244617 | 279229 | 169564 | 35, 4, 11 |
| 2 | DNPEP | aspartyl aminopeptidase | 123992 | 182698 | 175084 | 27, 3, 16 |
| 2 | DARS | aspartyl-tRNA synthetase | 115866 | 076003 | 121966 | 13, 1, 45 |
| 2 | ASTL | astacin like metalloendopeptidase | 188886 | 274286 | 158050 | 44, |
| 2 | ATL2 | atlastin GTPase 2 | 119787 | 138061 | 143889 | 13, 39, 42 |
| 2 | ATOH8 | atonal bHLH transcription factor 8 | 168874 | 115523 | 115525 | 3, 1, 45 |
| 2 | ABCA12 | ATP binding cassette subfamily A member 12 | 144452 | 138376 | 138363 | 24, 3, 20 |
| 2 | ABCB11 | ATP binding cassette subfamily B member 11 | 073734 | 152254 | 073737 | 42, 38, 19 |
| 2 | ABCB6 | ATP binding cassette subfamily B member 6 (Langereis blood group) | 115657 | 158552 | 198925 | 27, 7, 26 |
| 2 | ABCG5 | ATP binding cassette subfamily G member 5 | 138075 | 138036 | 143921 | 19, 37, 14 |
| 2 | ABCG8 | ATP binding cassette subfamily G member 8 | 143921 | 138075 | 138095 | 25, |
| 2 | ATP5G3 | ATP synthase, H+ transporting, mitochondrial Fo complex subunit C3 (subunit 9) | 154518 | 115966 | 144320 | 29, 11, 15 |
| 2 | AGBL5 | ATP/GTP binding protein like 5 | 084693 | 119777 | 228474 | 38, 9, 3 |
| 2 | ATAD2B | ATPase family, AAA domain containing 2B | 119778 | 119771 | 173960 | 39, 31, 32 |
| 2 | ATP6V1B1 | ATPase H+ transporting V1 subunit B1 | 116039 | 116035 | 258881 | 17, 23, 27 |
| 2 | ATP6V1C2 | ATPase H+ transporting V1 subunit C2 | 143882 | 115761 | 143870 | 11, 32, 40 |
| 2 | ATP6V1E2 | ATPase H+ transporting V1 subunit E2 | 250565 | 187600 | 119729 | 38, 9, 15 |
| 2 | ARID5A | AT-rich interaction domain 5A | 196843 | 163121 | 114982 | 26, 41, 5 |
| 2 | ACKR3 | atypical chemokine receptor 3 | 144476 | 132321 | 198612 | |
| 2 | ATG16L1 | autophagy related 16 like 1 | 085978 | 168918 | 130561 | 31, 34, 39 |
| 2 | ATG4B | autophagy related 4B cysteine peptidase | 168397 | 176946 | 168393 | 34, 38, 9 |
| 2 | ATG9A | autophagy related 9A | 198925 | 115657 | 163516 | 38, 8, 24 |
| 2 | BIRC6 | baculoviral IAP repeat containing 6 | 115760 | 119820 | 018699 | 10, 1, 23 |
| 2 | BBS5 | Bardet-Biedl syndrome 5 | 163093 | 081479 | 251569 | 38, 4, 2 |
| 2 | BZW1 | basic leucine zipper and W2 domains 1 | 082153 | 280298 | 013441 | 45, 4, 39 |
| 2 | BCL11A | B-cell CLL/lymphoma 11A | 119866 | 115392 | 115421 | 22, 29, 21 |
| 2 | BCL2L11 | BCL2 like 11 | 153094 | 153093 | 153107 | 22, 1, 26 |
| 2 | BCS1L | BCS1 homolog, ubiquinol-cytochrome c reductase complex chaperone | 074582 | 115568 | 163481 | |
| 2 | B3GALT1 | beta-1,3-galactosyltransferase 1 | 172318 | 163092 | 198648 | 10, |
| 2 | BOK | BOK, BCL2 family apoptosis regulator | 176720 | 115694 | 176946 | 1, 9, 28 |
| 2 | BOLA3 | bolA family member 3 | 163170 | 187605 | 114978 | 14, |
| 2 | BMP10 | bone morphogenetic protein 10 | 163217 | 163219 | 283586 | 3, |
| 2 | BMPR2 | bone morphogenetic protein receptor type 2 | 204217 | 055044 | 138439 | 45, 26, 16 |
| 2 | BOLL | boule homolog, RNA binding protein | 152430 | 247626 | 115896 | |
| 2 | BRE | brain and reproductive organ-expressed (TNFRSF1A modulator) | 158019 | 171174 | 075426 | |
| 2 | BARD1 | BRCA1 associated RING domain 1 | 138376 | 174453 | 144452 | 10, 22, 26 |
| 2 | BIN1 | bridging integrator 1 | 136717 | 237524 | 186684 | 34, 26, 25 |
| 2 | BAZ2B | bromodomain adjacent to zinc finger domain 2B | 123636 | 196151 | 136536 | 31, 24, 27 |
| 2 | BUB1 | BUB1 mitotic checkpoint serine/threonine kinase | 169679 | 183054 | 153093 | 39, 38, 7 |
| 2 | C1D | C1D nuclear receptor corepressor | 197223 | 143971 | 243667 | 34, 4, 7 |
| 2 | C1GALT1C1L | C1GALT1-specific chaperone 1 like | 223658 | 152527 | 138036 | 38, |
| 2 | CALCRL | calcitonin receptor like receptor | 064989 | 163012 | 003436 | 42, 45, 24 |
| 2 | CIB4 | calcium and integrin binding family member 4 | 157884 | 173557 | 171303 | 3, 38, 33 |
| 2 | CAB39 | calcium binding protein 39 | 135932 | 067066 | 135916 | 22, 40, 17 |
| 2 | CARF | calcium responsive transcription factor | 138380 | 138442 | 144426 | |
| 2 | CACNB4 | calcium voltage-gated channel auxiliary subunit beta 4 | 182389 | 283228 | 115145 | 8, 25, 22 |
| 2 | CALM2 | calmodulin 2 | 143933 | 273269 | 119888 | 4, 34, 31 |
| 2 | CAMKMT | calmodulin-lysine N-methyltransferase | 143919 | 138078 | 138083 | |
| 2 | CAPN10 | calpain 10 | 142330 | 142327 | 178623 | 21, 34, 39 |
| 2 | CAPN13 | calpain 13 | 162949 | 172954 | 158089 | 23, 24, 28 |
| 2 | CAPN14 | calpain 14 | 214711 | 158089 | 013016 | 24, 22, 30 |
| 2 | CREB1 | cAMP responsive element binding protein 1 | 118260 | 118263 | 144401 | 22, 4, 7 |
| 2 | CNRIP1 | cannabinoid receptor interacting protein 1 | 119865 | 221823 | 115956 | 26, 11, 14 |
| 2 | CLIP4 | CAP-Gly domain containing linker protein family member 4 | 115295 | 179270 | 171094 | 42, 31, 1 |
| 2 | CAPG | capping actin protein, gelsolin like | 042493 | 115459 | 152292 | 19, |
| 2 | CPS1 | carbamoyl-phosphate synthase 1 | 021826 | 115365 | 178568 | 19, 9, 36 |
| 2 | CAD | carbamoyl-phosphate synthetase 2, aspartate transcarbamylase, and | 084774 | 138085 | 115194 | 21, 7, 22 |
| | | dihydroorotase | | | | |
| 2 | CHST10 | carbohydrate sulfotransferase 10 | 115526 | 170500 | 204640 | 26, 40, 12 |
| 2 | CPO | carboxypeptidase O | 144410 | 118246 | 118263 | |
| 2 | CFLAR | CASP8 and FADD like apoptosis regulator | 003402 | 119013 | 003400 | 18, 21, 40 |
| 2 | CASP10 | caspase 10 | 003400 | 003402 | 064012 | 7, 26, 39 |
| 2 | CASP8 | caspase 8 | 064012 | 003400 | 155749 | 39, 30, 5 |
| 2 | CTNNA2 | catenin alpha 2 | 066032 | 172016 | 162951 | 8, 38, 26 |
| 2 | CCL20 | C-C motif chemokine ligand 20 | 115009 | 135917 | 123977 | 30, 16, 37 |
| 2 | CEBPZ | CCAAT/enhancer binding protein zeta | 115816 | 218739 | 003509 | 24, 38, 19 |
| 2 | CNOT11 | CCR4-NOT transcription complex subunit 11 | 158435 | 204634 | 163162 | 4, 7, 1 |
| 2 | CNOT9 | CCR4-NOT transcription complex subunit 9 | 144580 | 135913 | 115556 | |
| 2 | CD207 | CD207 molecule | 116031 | 152672 | 116035 | 4, 13, 40 |
| 2 | CD28 | CD28 molecule | 178562 | 173166 | 163599 | 1, 39, 30 |
| 2 | CD302 | CD302 molecule | 241399 | 136536 | 248672 | 42, 44, 7 |
| 2 | CD8A | CD8a molecule | 153563 | 153561 | 172116 | 39, 36, 5 |
| 2 | CD8B | CD8b molecule | 172116 | 153563 | 187627 | 39, 31, 27 |
| 2 | CLK1 | CDC like kinase 1 | 013441 | 082153 | 240344 | 39, 36, 42 |
| 2 | CDC42EP3 | CDC42 effector protein 3 | 163171 | 115828 | 115841 | |
| 2 | CEBPZOS | CEBPZ opposite strand | 218739 | 138068 | 115816 | 4, 7, 18 |
| 2 | CDCA7 | cell division cycle associated 7 | 144354 | 091436 | 172845 | 22, 4, 43 |
| 2 | CGREF1 | cell growth regulator with EF-hand domain 1 | 138028 | 138030 | 143994 | 3, 8, 14 |
| 2 | CREG2 | cellular repressor of E1A stimulated genes 2 | 175874 | 163162 | 196460 | 8, 6, 12 |
| 2 | CENPA | centromere protein A | 115163 | 213699 | 157851 | 9, 22, 6 |
| 2 | CENPO | centromere protein O | 138092 | 184924 | 138031 | 34, 26, 12 |
| 2 | CEP68 | centrosomal protein 68 | 011523 | 115902 | 138069 | 21, 1, 24 |
| 2 | CERKL | ceramide kinase like | 188452 | 115232 | 162992 | 15, 14, 27 |
| 2 | CERS6 | ceramide synthase 6 | 172292 | 198648 | 163072 | 40, 24, 16 |
| 2 | CHAC2 | ChaC cation transport regulator homolog 2 | 143942 | 270898 | 068912 | 34, 22, 4 |
| 2 | CCT4 | chaperonin containing TCP1 subunit 4 | 115484 | 170264 | 173163 | 13, 10, 29 |
| 2 | CCT7 | chaperonin containing TCP1 subunit 7 | 135624 | 135617 | 163013 | 13, 21, 9 |
| 2 | CHMP3 | charged multivesicular body protein 3 | 115561 | 115548 | 249884 | 26, 45, 4 |
| 2 | CHN1 | chimerin 1 | 128656 | 138435 | 115966 | 8, 30, 17 |
| 2 | CHRNA1 | cholinergic receptor nicotinic alpha 1 subunit | 138435 | 115935 | 128656 | |
| 2 | CHRND | cholinergic receptor nicotinic delta subunit | 135902 | 237412 | 196811 | 15, 25, 14 |
| 2 | CHRNG | cholinergic receptor nicotinic gamma subunit | 196811 | 135902 | 221944 | 24, |
| 2 | CHPF | chondroitin polymerizing factor | 123989 | 072182 | 188760 | 35, 18, 31 |
| 2 | C2orf15 | chromosome 2 open reading frame 15 | 273045 | 135951 | 144182 | 4, 40, 32 |
| 2 | C2orf16 | chromosome 2 open reading frame 16 | 221843 | 280034 | 243943 | 38, 16, 12 |
| 2 | C2orf40 | chromosome 2 open reading frame 40 | 119147 | 071051 | 115652 | 11, 10, 26 |
| 2 | C2orf42 | chromosome 2 open reading frame 42 | 115998 | 169564 | 116001 | 38, 36, 3 |
| 2 | C2orf49 | chromosome 2 open reading frame 49 | 135974 | 135966 | 115641 | 22, 26, 17 |
| 2 | C2orf50 | chromosome 2 open reading frame 50 | 150873 | 162975 | 162976 | 42, 38, 8 |
| 2 | C2orf54 | chromosome 2 open reading frame 54 | 172478 | 172482 | 226321 | 13, 24, 23 |
| 2 | C2orf57 | Chromosome 2 open reading frame 57 | 177673 | 171596 | 187514 | 38, 8, |
| 2 | C2orf61 | chromosome 2 open reading frame 61 | 239605 | 068724 | 273269 | 38, 39, 8 |
| 2 | C2orf66 | chromosome 2 open reading frame 66 | 187944 | 119041 | 197121 | 31, 2, 10 |
| 2 | C2orf68 | chromosome 2 open reading frame 68 | 168887 | 168883 | 168878 | 18, 30, 29 |
| 2 | C2orf69 | chromosome 2 open reading frame 69 | 178074 | 226124 | 162971 | 42, 43, 22 |
| 2 | C2orf70 | chromosome 2 open reading frame 70 | 173557 | 115155 | 157884 | 38, 16, 8 |
| 2 | C2orf71 | chromosome 2 open reading frame 71 | 179270 | 189350 | 115295 | |
| 2 | C2orf72 | chromosome 2 open reading frame 72 | 204128 | 173699 | 173692 | 26, 32, 19 |
| 2 | C2orf73 | chromosome 2 open reading frame 73 | 177994 | 178021 | 115306 | |
| 2 | C2orf74 | chromosome 2 open reading frame 74 | 237651 | 162929 | 173209 | 11, 27, 23 |
| 2 | C2orf76 | chromosome 2 open reading frame 76 | 186132 | 115107 | 155368 | 10, 22, 29 |
| 2 | C2orf78 | chromosome 2 open reading frame 78 | 187833 | 144048 | 124356 | 38, 25, |
| 2 | C2orf80 | chromosome 2 open reading frame 80 | 188674 | 168582 | 138413 | 8, 38, 10 |
| 2 | C2orf81 | chromosome 2 open reading frame 81 | 159239 | 204843 | 005448 | 17, 21, 38 |
| 2 | C2orf82 | chromosome 2 open reading frame 82 | 182600 | 115474 | 066248 | |
| 2 | C2orf83 | chromosome 2 open reading frame 83 | 042304 | 173744 | 135917 | 32, 8, |
| 2 | C2orf88 | chromosome 2 open reading frame 88 | 187699 | 064933 | 138379 | 38, 26, 44 |
| 2 | C2orf91 | chromosome 2 open reading frame 91 | 205086 | 183023 | 162878 | |
| 2 | CFAP221 | cilia and flagella associated protein 221 | 163075 | 080293 | 144120 | |
| 2 | CFAP36 | cilia and flagella associated protein 36 | 163001 | 115355 | 275052 | |
| 2 | CFAP65 | cilia and flagella associated protein 65 | 181378 | 163499 | 163501 | 26, 38, 42 |
| 2 | CROCC2 | ciliary rootlet coiled-coil, rootletin family member 2 | 226321 | 172478 | 162804 | |
| 2 | CATIP | ciliogenesis associated TTC17 interacting protein | 158428 | 135926 | 018280 | 32, 38, 12 |
| 2 | CLHC1 | clathrin heavy chain linker domain containing 1 | 162994 | 115310 | 143947 | 11, 26, 38 |
| 2 | CPSF3 | cleavage and polyadenylation specific factor 3 | 119203 | 119185 | 134330 | 10, 36, 1 |
| 2 | CBWD2 | COBW domain containing 2 | 136682 | 231292 | 184492 | |
| 2 | COQ10B | coenzyme Q10B | 115520 | 115524 | 144381 | 2, 43, 37 |
| 2 | CCDC115 | coiled-coil domain containing 115 | 136710 | 152086 | 136718 | 21, 41, 5 |
| 2 | CCDC121 | coiled-coil domain containing 121 | 176714 | 243943 | 198522 | 38, 22, 35 |
| 2 | CCDC138 | coiled-coil domain containing 138 | 163006 | 153201 | 135960 | |
| 2 | CCDC140 | coiled-coil domain containing 140 | 163081 | 135903 | 163082 | 25, |
| 2 | CCDC141 | coiled-coil domain containing 141 | 163492 | 283186 | 187231 | 2, 15, 14 |
| 2 | CCDC142 | coiled-coil domain containing 142 | 135637 | 204822 | 115282 | 24, 39, 36 |
| 2 | CCDC148 | coiled-coil domain containing 148 | 153237 | 007001 | 144283 | 10, 4, 42 |
| 2 | CCDC150 | coiled-coil domain containing 150 | 144395 | 138411 | 119041 | 16, 13, 12 |
| 2 | CCDC173 | coiled-coil domain containing 173 | 154479 | 138398 | 144362 | 38, 42, 10 |
| 2 | CCDC74A | coiled-coil domain containing 74A | 163040 | 075886 | 163046 | 43, 38, 21 |
| 2 | CCDC74B | coiled-coil domain containing 74B | 152076 | 196604 | 136699 | 38, 26, 31 |
| 2 | CCDC85A | coiled-coil domain containing 85A | 055813 | 115380 | 028116 | 10, 22, 7 |
| 2 | CCDC88A | coiled-coil domain containing 88A | 115355 | 085760 | 163001 | 22, 7, 26 |
| 2 | CCDC93 | coiled-coil domain containing 93 | 125633 | 088205 | 125629 | 18, 22, 41 |
| 2 | CHCHD5 | coiled-coil-helix-coiled-coil-helix domain containing 5 | 125611 | 125630 | 144136 | 29, 35, 41 |
| 2 | COL3A1 | collagen type III alpha 1 chain | 168542 | 174325 | 204262 | 11, 12, 43 |
| 2 | COL4A3 | collagen type IV alpha 3 chain | 169031 | 081052 | 168958 | 31, 14, 40 |
| 2 | COL4A4 | collagen type IV alpha 4 chain | 081052 | 144468 | 169031 | 31, 2, 17 |
| 2 | COL5A2 | collagen type V alpha 2 chain | 204262 | 168542 | 115368 | 12, 6, 11 |
| 2 | COL6A3 | collagen type VI alpha 3 chain | 163359 | 198612 | 115648 | 6, 1, 10 |
| 2 | COLEC11 | collectin subfamily member 11 | 118004 | 171863 | 151360 | 27, 8, 38 |
| 2 | C1QL2 | complement C1q like 2 | 144119 | 019169 | 115107 | 14, 8, 20 |
| 2 | CNTNAP5 | contactin associated protein like 5 | 155052 | 211460 | 136732 | 14, 8, 38 |
| 2 | COPS7B | COP9 signalosome subunit 7B | 144524 | 156973 | 163273 | 36, 39, 41 |
| 2 | COPS8 | COP9 signalosome subunit 8 | 198612 | 144476 | 163359 | 29, 30, 3 |
| 2 | COPS9 | COP9 signalosome subunit 9 | 172428 | 178586 | 178602 | |
| 2 | COMMD1 | copper metabolism domain containing 1 | 173163 | 115484 | 280544 | 10, 30, 15 |
| 2 | COBLL1 | cordon-bleu WH2 repeat protein like 1 | 082438 | 115290 | 169507 | 32, 22, 24 |
| 2 | CIR1 | corepressor interacting with RBPJ, 1 | 138433 | 217236 | 144306 | 11, 29, 6 |
| 2 | CFC1 | cripto, FRL-1, cryptic family 1 | 136698 | 183292 | 222038 | 28, 8, 20 |
| 2 | CFC1B | cripto, FRL-1, cryptic family 1B | 152093 | 196834 | 184761 | 20, 28, 14 |
| 2 | CRYBA2 | crystallin beta A2 | 163499 | 163497 | 181378 | 14, 28, 31 |
| 2 | CRYGA | crystallin gamma A | 168582 | 182187 | 188674 | 14, 10, |
| 2 | CRYGB | crystallin gamma B | 182187 | 163254 | 168582 | 14, |
| 2 | CRYGC | crystallin gamma C | 163254 | 118231 | 182187 | 14, 10, 38 |
| 2 | CRYGD | crystallin gamma D | 118231 | 178385 | 163254 | 14, 27, 12 |
| 2 | CTDSP1 | CTD small phosphatase 1 | 144579 | 018280 | 127831 | 34, 41, 9 |
| 2 | CLEC4F | C-type lectin domain family 4 member F | 152672 | 183733 | 116031 | 24, 30, 14 |
| 2 | CUL3 | cullin 3 | 036257 | 124019 | 283428 | 29, 30, 22 |
| 2 | CWC22 | CWC22 spliceosome associated protein homolog | 163510 | 144331 | 170035 | 4, 1, 30 |
| 2 | CXCR1 | C-X-C motif chemokine receptor 1 | 163464 | 180871 | 163466 | 5, 36, 39 |
| 2 | CXCR2 | C-X-C motif chemokine receptor 2 | 180871 | 188282 | 163464 | 5, 36, 2 |
| 2 | CXCR4 | C-X-C motif chemokine receptor 4 | 121966 | 115866 | 144229 | 2, 5, 36 |
| 2 | CRIPT | CXXC repeat containing interactor of PDZ3 domain | 119878 | 151665 | 171150 | 4, 34, 7 |
| 2 | CNGA3 | cyclic nucleotide gated channel alpha 3 | 144191 | 168658 | 040933 | 31, 14, 8 |
| 2 | CNNM3 | cyclin and CBS domain divalent metal cation transport mediator 3 | 168763 | 158158 | 163126 | 21, 14, 28 |
| 2 | CNNM4 | cyclin and CBS domain divalent metal cation transport mediator 4 | 158158 | 114988 | 168763 | 9, 22, 31 |
| 2 | CDK15 | cyclin dependent kinase 15 | 138395 | 003393 | 155760 | 1, 35, 38 |
| 2 | CDK5R2 | cyclin dependent kinase 5 regulatory subunit 2 | 171450 | 135925 | 163497 | 28, 2, 8 |
| 2 | CDKL4 | cyclin dependent kinase like 4 | 205111 | 115904 | 011566 | 38, 20, |
| 2 | CNPPD1 | cyclin Pas1/PHO80 domain containing 1 | 115649 | 213901 | 144567 | 29, 41, 27 |
| 2 | CCNT2 | cyclin T2 | 082258 | 153086 | 176601 | 42, 22, 30 |
| 2 | CCNYL1 | cyclin Y like 1 | 163249 | 144401 | 163251 | 26, 13, 29 |
| 2 | CSRNP3 | cysteine and serine rich nuclear protein 3 | 178662 | 136531 | 115339 | 11, 8, 1 |
| 2 | CRIM1 | cysteine rich transmembrane BMP regulator 1 | 150938 | 119812 | 171055 | 45, 31, 32 |
| 2 | CYS1 | cystin 1 | 205795 | 172059 | 171848 | 17, 44, 14 |
| 2 | CMPK2 | cytidine/uridine monophosphate kinase 2 | 134326 | 176887 | 134321 | 7, 34, 4 |
| 2 | CYBRD1 | cytochrome b reductase 1 | 071967 | 115827 | 077380 | 42, 1, 44 |
| 2 | COA5 | cytochrome c oxidase assembly factor 5 | 183513 | 040933 | 115446 | 17, 15, 11 |
| 2 | COX5B | cytochrome c oxidase subunit 5B | 135940 | 196912 | 115073 | 19, 25, 43 |
| 2 | COX7A2L | cytochrome c oxidase subunit 7A2 like | 115944 | 143924 | 171126 | |
| 2 | CYP1B1 | cytochrome P450 family 1 subfamily B member 1 | 138061 | 115841 | 119787 | |
| 2 | CYP20A1 | cytochrome P450 family 20 subfamily A member 1 | 119004 | 144426 | 138443 | 29, 2, 40 |
| 2 | CYP26B1 | cytochrome P450 family 26 subfamily B member 1 | 003137 | 135636 | 144036 | 1, 39, 2 |
| 2 | CYP27A1 | cytochrome P450 family 27 subfamily A member 1 | 135929 | 135912 | 115592 | 5, 19, 35 |
| 2 | CYP27C1 | cytochrome P450 family 27 subfamily C member 1 | 186684 | 136717 | 163161 | 24, 10, 44 |
| 2 | CYTIP | cytohesin 1 interacting protein | 115165 | 136541 | 123612 | 30, 22, 5 |
| 2 | CLASP1 | cytoplasmic linker associated protein 1 | 074054 | 115112 | 155438 | 23, 39, 18 |
| 2 | CKAP2L | cytoskeleton associated protein 2 like | 169607 | 144130 | 115008 | 22, 26, 39 |
| 2 | CIAO1 | cytosolic iron-sulfur assembly component 1 | 144021 | 135956 | 144028 | 35, 23, 3 |
| 2 | CTLA4 | cytotoxic T-lymphocyte associated protein 4 | 163599 | 178562 | 163600 | 22, 5, 33 |
| 2 | D2HGDH | D-2-hydroxyglutarate dehydrogenase | 180902 | 168395 | 154252 | |
| 2 | DCAF17 | DDB1 and CUL4 associated factor 17 | 115827 | 123600 | 071967 | 25, 39, 34 |
| 2 | DDX1 | DEAD-box helicase 1 | 079785 | 151779 | 134323 | 14, 40, 8 |
| 2 | DDX18 | DEAD-box helicase 18 | 088205 | 175497 | 125633 | 42, 22, 40 |
| 2 | DFNB59 | deafness, autosomal recessive 59 | 204311 | 180228 | 079150 | 40, 22, 38 |
| 2 | DQX1 | DEAQ-box RNA dependent ATPase 1 | 144045 | 115297 | 115307 | 18, 3, 13 |
| 2 | DAPL1 | death associated protein like 1 | 163331 | 281472 | 115183 | 14, 40, 15 |
| 2 | DOCK10 | dedicater of cytokinesis 10 | 135905 | 283428 | 144460 | 18, 1, 36 |
| 2 | DHRS9 | dehydrogenase/reductase 9 | 073737 | 073734 | 081479 | 42, 30, 22 |
| 2 | DNER | delta/notch like EGF repeat containing | 187957 | 153823 | 153827 | 11, 31, 8 |
| 2 | DGUOK | deoxyguanosine kinase | 114956 | 163017 | 187605 | 30, 29, 15 |
| 2 | DTYMK | deoxythymidylate kinase | 168393 | 168397 | 168395 | 22, 25, 9 |
| 2 | DES | desmin | 175084 | 123992 | 072195 | 15, 18, 25 |
| 2 | DHX57 | DExH-box helicase 57 | 163214 | 152147 | 188010 | 43, 26, 31 |
| 2 | DGKD | diacylglycerol kinase delta | 077044 | 130561 | 085982 | 22, 21, 1 |
| 2 | DBI | diazepam binding inhibitor, acyl-CoA binding protein | 155368 | 186132 | 171227 | 29, 33, 19 |
| 2 | DPYSL5 | dihydropyrimidinase like 5 | 157851 | 115163 | 084764 | 8, 14, 12 |
| 2 | DPP4 | dipeptidyl peptidase 4 | 197635 | 144290 | 115263 | 29, 32, 1 |
| 2 | DPP10 | dipeptidyl peptidase like 10 | 175497 | 115091 | 088205 | 8, 4, 2 |
| 2 | DIS3L2 | DIS3 like 3′-5′ exoribonuclease 2 | 144535 | 163273 | 163283 | 26, 1, 7 |
| 2 | DIRC1 | disrupted in renal carcinoma 1 | 174325 | 144366 | 168542 | 10, |
| 2 | DIRC3 | Disrupted in renal carcinoma 3 | 231672 | 278951 | 079308 | 6, 45, 38 |
| 2 | DLX1 | distal-less homeobox 1 | 144355 | 172878 | 115844 | 6, 24, 8 |
| 2 | DLX2 | distal-less homeobox 2 | 115844 | 144355 | 091409 | 30, 5, 40 |
| 2 | DNMT3A | DNA methyltransferase 3 alpha | 119772 | 115138 | 138101 | 18, 16, 12 |
| 2 | POLE4 | DNA polymerase epsilon 4, accessory subunit | 115350 | 159399 | 115353 | 2, 35, 14 |
| 2 | DNAJB2 | DnaJ heat shock protein family (Hsp40) member B2 | 135924 | 243910 | 054356 | 26, 40, 31 |
| 2 | DNAJC10 | DnaJ heat shock protein family (Hsp40) member C10 | 077232 | 115252 | 162998 | 22, 43, 13 |
| 2 | DNAJC27 | DnaJ heat shock protein family (Hsp40) member C27 | 115137 | 138031 | 084710 | 14, 38, 28 |
| 2 | DNAJC5G | DnaJ heat shock protein family (Hsp40) member C5 gamma | 163793 | 115194 | 138100 | 38, 12, 8 |
| 2 | DOK1 | docking protein 1 | 115325 | 115318 | 159374 | |
| 2 | DCDC2C | doublecortin domain containing 2C | 214866 | 151360 | 176887 | |
| 2 | DPY30 | dpy-30, histone methyltransferase complex regulatory subunit | 162961 | 162959 | 021574 | 29, 4, 11 |
| 2 | DUSP11 | dual specificity phosphatase 11 | 144048 | 144034 | 187833 | 13, 30, 7 |
| 2 | DUSP19 | dual specificity phosphatase 19 | 162999 | 061676 | 163002 | 29, 38, 6 |
| 2 | DUSP2 | dual specificity phosphatase 2 | 158050 | 188886 | 084090 | 9, 5, 26 |
| 2 | DUSP28 | dual specificity phosphatase 28 | 188542 | 144504 | 142327 | 28, 38, 27 |
| 2 | DCTN1 | dynactin subunit 1 | 204843 | 188687 | 159239 | |
| 2 | DAW1 | dynein assembly factor with WD repeats 1 | 123977 | 115009 | 153820 | 38, 30, 45 |
| 2 | DNAH6 | dynein axonemal heavy chain 6 | 115423 | 163541 | 186854 | 31, 42, 6 |
| 2 | DNAH7 | dynein axonemal heavy chain 7 | 118997 | 196950 | 081320 | 31, 10, 38 |
| 2 | DYNC1I2 | dynein cytoplasmic 1 intermediate chain 2 | 077380 | 071967 | 115840 | 45, 8, 13 |
| 2 | DYNC2LI1 | dynein cytoplasmic 2 light intermediate chain 1 | 138036 | 223658 | 138075 | 4, 43, 17 |
| 2 | DRC1 | dynein regulatory complex subunit 1 | 157856 | 138018 | 115155 | 38, 42, 27 |
| 2 | DYSF | dysferlin | 135636 | 075292 | 003137 | 43, 5, 36 |
| 2 | DTNB | dystrobrevin beta | 138101 | 119772 | 143970 | 9, 26, 18 |
| 2 | DYTN | dystrotelin | 232125 | 235118 | 138400 | 38, |
| 2 | E2F6 | E2F transcription factor 6 | 169016 | 134318 | 196208 | 29, 31, 2 |
| 2 | EGR4 | early growth response 4 | 135625 | 163013 | 116127 | 20, 38, 32 |
| 2 | EML4 | echinoderm microtubule associated protein like 4 | 143924 | 162878 | 115944 | 26, 3, 23 |
| 2 | EML6 | echinoderm microtubule associated protein like 6 | 214595 | 115306 | 115310 | |
| 2 | EDAR | ectodysplasin A receptor | 135960 | 163006 | 172985 | 24, 15, 33 |
| 2 | EFHD1 | EF-hand domain family member D1 | 115468 | 135930 | 204120 | |
| 2 | EFR3B | EFR3 homolog B | 084710 | 115137 | 115138 | 29, 15, 28 |
| 2 | EFEMP1 | EGF containing fibulin like extracellular matrix protein 1 | 115380 | 138035 | 055813 | 1, 45, 40 |
| 2 | EHBP1 | EH domain binding protein 1 | 115504 | 186889 | 115507 | 1, 31, 38 |
| 2 | EHD3 | EH domain containing 3 | 013016 | 214711 | 158125 | 22, 17, 31 |
| 2 | EMILIN1 | elastin microfibril interfacer 1 | 138080 | 228474 | 138030 | 6, 1, 34 |
| 2 | ELMOD3 | ELMO domain containing 3 | 115459 | 042445 | 042493 | 1, 34, 22 |
| 2 | EMX1 | empty spiracles homeobox 1 | 135638 | 116096 | 144040 | 17, 8, 23 |
| 2 | ERLEC1 | endoplasmic reticulum lectin 1 | 068912 | 143942 | 119737 | 25, 43, 40 |
| 2 | EPAS1 | endothelial PAS domain protein 1 | 116016 | 171132 | 187600 | 42, 32, 29 |
| 2 | ECEL1 | endothelin converting enzyme like 1 | 171551 | 163295 | 237412 | 41, 31, 26 |
| 2 | EN1 | engrailed homeobox 1 | 163064 | 125629 | 019169 | 6, 32, 20 |
| 2 | EPC2 | enhancer of polycomb homolog 2 | 135999 | 204406 | 168280 | 32, 15, 2 |
| 2 | EPHA4 | EPH receptor A4 | 116106 | 114923 | 135903 | 40, 24, 8 |
| 2 | EPCAM | epithelial cell adhesion molecule | 119888 | 143933 | 095002 | 16, 37, 29 |
| 2 | ERBB4 | erb-b2 receptor tyrosine kinase 4 | 178568 | 021826 | 030419 | 29, 42, 25 |
| 2 | ERCC3 | ERCC excision repair 3, TFIIH core complex helicase subunit | 163161 | 186684 | 169967 | 1, 39, 3 |
| 2 | ERMN | ermin | 136541 | 136542 | 115165 | 8, 17, 22 |
| 2 | EPB41L5 | erythrocyte membrane protein band 4.1 like 5 | 115109 | 088179 | 226479 | 38, 17, 12 |
| 2 | ERFE | erythroferrone | 178752 | 168427 | 132323 | 38, 28, 6 |
| 2 | ESPNL | espin-like | 144488 | 132330 | 168427 | 31, 21, 32 |
| 2 | EEF1B2 | eukaryotic translation elongation factor 1 beta 2 | 114942 | 023228 | 183671 | 41, 43, 34 |
| 2 | EIF2AK2 | eukaryotic translation initiation factor 2 alpha kinase 2 | 055332 | 152133 | 138068 | 37, 16, 11 |
| 2 | EIF2AK3 | eukaryotic translation initiation factor 2 alpha kinase 3 | 172071 | 172073 | 153574 | 22, 42, 4 |
| 2 | EIF2B4 | eukaryotic translation initiation factor 2B subunit delta | 115211 | 115207 | 115234 | 14, 21, 25 |
| 2 | EIF4E2 | eukaryotic translation initiation factor 4E family member 2 | 135930 | 221944 | 115468 | 4, 3, 25 |
| 2 | EIF5B | eukaryotic translation initiation factor 5B | 158417 | 115514 | 135945 | 27, 5, 7 |
| 2 | EVA1A | eva-1 homolog A, regulator of programmed cell death | 115363 | 115353 | 115364 | 45, 17, 19 |
| 2 | EVX2 | even-skipped homeobox 2 | 174279 | 144320 | 128714 | 27, |
| 2 | ETAA1 | Ewing tumor associated antigen 1 | 143971 | 143995 | 197223 | 1, 22, 20 |
| 2 | EXOC6B | exocyst complex component 6B | 144036 | 003137 | 116096 | 32, 18, 26 |
| 2 | XPO1 | exportin 1 | 082898 | 115464 | 170264 | 13, 42, 12 |
| 2 | FAM110C | family with sequence similarity 110 member C | 184731 | 115738 | 035115 | 40, 45, 5 |
| 2 | FAM117B | family with sequence similarity 117 member B | 138439 | 204217 | 163596 | 18, 1, 27 |
| 2 | FAM124B | family with sequence similarity 124 member B | 124019 | 135919 | 036257 | 45, 43, 25 |
| 2 | FAM126B | family with sequence similarity 126 member B | 155744 | 115942 | 119013 | 11, 39, 32 |
| 2 | FAM134A | family with sequence similarity 134 member A | 144567 | 115649 | 158552 | 38, 1, 6 |
| 2 | FAM136A | family with sequence similarity 136 member A | 035141 | 143977 | 163235 | 3, 30, 13 |
| 2 | FAM150B | family with sequence similarity 150 member B | 189292 | 143727 | 151353 | 31, 45, 28 |
| 2 | FAM161A | family with sequence similarity 161 member A | 170264 | 082898 | 115484 | 14, 38, 2 |
| 2 | FAM168B | family with sequence similarity 168 member B | 152102 | 136002 | 115762 | 34, 26, 29 |
| 2 | FAM171B | family with sequence similarity 171 member B | 144369 | 138448 | 163012 | 11, 31, 4 |
| 2 | FAM178B | family with sequence similarity 178 member B | 168754 | 168758 | 144199 | 7, 36, 38 |
| 2 | FAM179A | family with sequence similarity 179 member A | 189350 | 163811 | 179270 | 40, 15, 28 |
| 2 | FAM228A | family with sequence similarity 228 member A | 186453 | 276087 | 198399 | |
| 2 | FAM228B | family with sequence similarity 228 member B | 219626 | 115128 | 115129 | |
| 2 | FAM49A | family with sequence similarity 49 member A | 197872 | 134323 | 214842 | 8, 22, 1 |
| 2 | FAM84A | family with sequence similarity 84 member A | 162981 | 071575 | 151779 | |
| 2 | FAM98A | family with sequence similarity 98 member A | 119812 | 152689 | 150938 | 7, 17, 3 |
| 2 | FANCL | Fanconi anemia complementation group L | 115392 | 028116 | 119866 | 11, 16, 22 |
| 2 | FEZ2 | fasciculation and elongation protein zeta 2 | 171055 | 150938 | 205221 | 25, 18, 7 |
| 2 | FASTKD1 | FAST kinase domains 1 | 138399 | 239474 | 138398 | 24, 2, 45 |
| 2 | FASTKD2 | FAST kinase domains 2 | 118246 | 138400 | 144410 | |
| 2 | FABP1 | fatty acid binding protein 1 | 163586 | 115593 | 144115 | 19, 16, 36 |
| 2 | FBXO11 | F-box protein 11 | 138081 | 116062 | 170802 | 29, 42, 22 |
| 2 | FBXO36 | F-box protein 36 | 153832 | 153827 | 163053 | 1, 30, 10 |
| 2 | FBXO41 | F-box protein 41 | 163013 | 135624 | 135625 | 21, 22, 26 |
| 2 | FBXO48 | F-box protein 48 | 204923 | 115956 | 169621 | 30, 22, 17 |
| 2 | FER1L5 | fer-1 like family member 5 | 249715 | 114982 | 114988 | 31, 38, 28 |
| 2 | FARP2 | FERM, ARH/RhoGEF and pleckstrin domain protein 2 | 006607 | 168385 | 115694 | 43, 29, 22 |
| 2 | FEV | FEV, ETS transcription factor | 163497 | 171450 | 163499 | 28, 33, 8 |
| 2 | FAP | fibroblast activation protein alpha | 078098 | 115263 | 115267 | |
| 2 | FN1 | fibronectin 1 | 115414 | 138363 | 118242 | 10, 6, 7 |
| 2 | FNDC4 | fibronectin type III domain containing 4 | 115226 | 138002 | 084734 | 26, 2, 15 |
| 2 | FSIP2 | fibrous sheath interacting protein 2 | 188738 | 170396 | 065548 | 38, 30, 12 |
| 2 | FBLN7 | fibulin 7 | 144152 | 153214 | 144161 | 1, 32, 27 |
| 2 | FIGN | fidgetin, microtubule severing factor | 182263 | 184611 | 115290 | 40, 4, 45 |
| 2 | FKBP1B | FK506 binding protein 1B | 119782 | 163026 | 115128 | 26, 2, 44 |
| 2 | FKBP7 | FK506 binding protein 7 | 079150 | 204311 | 116095 | 31, 10, 6 |
| 2 | FSHR | follicle stimulating hormone receptor | 170820 | 138039 | 179915 | 38, |
| 2 | FIGLA | folliculogenesis specific bHLH transcription factor | 183733 | 075340 | 152672 | |
| 2 | FOXD4L1 | forkhead box D4-like 1 | 184492 | 136682 | 144134 | 39, 24, 44 |
| 2 | FOXI3 | forkhead box I3 | 214336 | 144115 | 172073 | 14, |
| 2 | FOXN2 | forkhead box N2 | 170802 | 138081 | 162869 | 4, 29, 27 |
| 2 | FTCDNL1 | Formiminotransferase cyclodeaminase N-terminal like | 226124 | 119042 | 178074 | 6, 17, 40 |
| 2 | FMNL2 | formin like 2 | 157827 | 115145 | 196504 | 31, 40, 3 |
| 2 | FOSL2 | FOS like 2, AP-1 transcription factor subunit | 075426 | 158019 | 163803 | 34, 4, 26 |
| 2 | FHL2 | four and a half LIM domains 2 | 115641 | 135974 | 071051 | 15, 34, 37 |
| 2 | FZD5 | frizzled class receptor 5 | 163251 | 163249 | 178385 | 6, 12, 18 |
| 2 | FZD7 | frizzled class receptor 7 | 155760 | 138395 | 182329 | 12, 27, 6 |
| 2 | FRZB | frizzled-related protein | 162998 | 077232 | 061676 | 11, 14, 36 |
| 2 | FAHD2A | fumarylacetoacetate hydrolase domain containing 2A | 115042 | 115041 | 144015 | |
| 2 | FAHD2B | fumarylacetoacetate hydrolase domain containing 2B | 144199 | 168754 | 135976 | 43, 13, 29 |
| 2 | GPBAR1 | G protein-coupled bile acid receptor 1 | 179921 | 163466 | 127837 | 26, 36, 32 |
| 2 | GPR1 | G protein-coupled receptor 1 | 183671 | 114942 | 204186 | 1, 10, 43 |
| 2 | GPR148 | G protein-coupled receptor 148 | 173302 | 222038 | 178171 | |
| 2 | GPR155 | G protein-coupled receptor 155 | 163328 | 144306 | 115935 | 26, 11, 31 |
| 2 | GPR17 | G protein-coupled receptor 17 | 144230 | 072163 | 136709 | |
| 2 | GPR35 | G protein-coupled receptor 35 | 178623 | 142330 | 185176 | 16, 37, 36 |
| 2 | GPR39 | G protein-coupled receptor 39 | 183840 | 163046 | 150551 | 17, 6, 38 |
| 2 | GPR45 | G protein-coupled receptor 45 | 135973 | 135972 | 135966 | 20, |
| 2 | GPR55 | G protein-coupled receptor 55 | 135898 | 135916 | 173699 | 3, 36, 38 |
| 2 | GPR75 | G protein-coupled receptor 75 | 119737 | 068912 | 068878 | |
| 2 | GALM | galactose mutarotase | 143891 | 143889 | 115875 | 2, 17, 4 |
| 2 | GAL3ST2 | galactose-3-O-sulfotransferase 2 | 154252 | 180902 | 204099 | 14, |
| 2 | GLB1L | galactosidase beta 1 like | 163521 | 163516 | 115661 | 38, 36, 26 |
| 2 | LGALSL | galectin like | 119862 | 197329 | 119844 | 29, 13, 14 |
| 2 | GGCX | gamma-glutamyl carboxylase | 115486 | 168906 | 118640 | 24, 29, 32 |
| 2 | GKN1 | gastrokine 1 | 169605 | 183607 | 169604 | 37, 44, 32 |
| 2 | GKN2 | gastrokine 2 | 183607 | 283586 | 169605 | 37, 10, 20 |
| 2 | GKN3P | Gastrokine 3, pseudogene | 283586 | 163217 | 183607 | |
| 2 | GBX2 | gastrulation brain homeobox 2 | 168505 | 157985 | 182177 | 37, 12, 25 |
| 2 | GCFC2 | GC-rich sequence DNA-binding factor 2 | 005436 | 115364 | 176204 | 26, 11, 34 |
| 2 | GMPPA | GDP-mannose pyrophosphorylase A | 144591 | 072195 | 072182 | 7, 3, 5 |
| 2 | GEMIN6 | gem nuclear organelle associated protein 6 | 152147 | 115875 | 163214 | 1, 3, 41 |
| 2 | GEN1 | GEN1, Holliday junction 5′ flap endonuclease | 178295 | 163029 | 151379 | 22, 30, 29 |
| 2 | GTF2A1L | general transcription factor IIA subunit 1 like | 242441 | 068781 | 279956 | 38, 43, 4 |
| 2 | GTF3C2 | general transcription factor IIIC subunit 2 | 115207 | 115204 | 115211 | 43, 29, 24 |
| 2 | GTF3C3 | general transcription factor IIIC subunit 3 | 119041 | 144395 | 187944 | 18, 42, 26 |
| 2 | GMCL1 | germ cell-less, spermatogenesis associated 1 | 087338 | 196975 | 124380 | 6, 37, 28 |
| 2 | GLI2 | GLI family zinc finger 2 | 074047 | 163083 | 115112 | 15, 7, 6 |
| 2 | GCG | glucagon | 115263 | 197635 | 078098 | |
| 2 | GCKR | glucokinase regulator | 084734 | 115226 | 233438 | 19, 45, 2 |
| 2 | G6PC2 | glucose-6-phosphatase catalytic subunit 2 | 152254 | 152253 | 073734 | 28, 42, 25 |
| 2 | GAD1 | glutamate decarboxylase 1 | 128683 | 204334 | 115806 | 23, 8, 16 |
| 2 | ERICH2 | glutamate rich 2 | 204334 | 204335 | 128683 | 38, 32, 23 |
| 2 | GLS | glutaminase | 115419 | 138386 | 115415 | |
| 2 | GFPT1 | glutamine--fructose-6-phosphate transaminase 1 | 198380 | 169604 | 169599 | 34, 16, 7 |
| 2 | QPCT | glutaminyl-peptide cyclotransferase | 115828 | 115825 | 163171 | 2, 10, 35 |
| 2 | GPAT2 | glycerol-3-phosphate acyltransferase 2, mitochondrial | 186281 | 174501 | 274286 | 38, 21, 15 |
| 2 | GPD2 | glycerol-3-phosphate dehydrogenase 2 | 115159 | 153234 | 136542 | 11, 42, 24 |
| 2 | GYPC | glycophorin C (Gerbich blood group) | 136732 | 155052 | 237524 | 22, 35, 25 |
| 2 | GTDC1 | glycosyltransferase like domain containing 1 | 121964 | 075884 | 169554 | 29, 22, 1 |
| 2 | GPC1 | glypican 1 | 063660 | 178602 | 144504 | 25, 9, 34 |
| 2 | GORASP2 | golgi reassembly stacking protein 2 | 115806 | 128683 | 198586 | 45, 3, 2 |
| 2 | GPATCH11 | G-patch domain containing 11 | 152133 | 008869 | 055332 | 29, 26, 7 |
| 2 | GPN1 | GPN-loop GTPase 1 | 198522 | 176714 | 119760 | 33, 43, 11 |
| 2 | GPR75-ASB3 | GPR75-ASB3 readthrough | 270898 | 115239 | 143942 | |
| 2 | GRHL1 | grainyhead like transcription factor 1 | 134317 | 115750 | 172059 | 18, 24, 42 |
| 2 | GCA | grancalcin | 115271 | 115267 | 184611 | 5, 43, 7 |
| 2 | GNLY | granulysin | 115523 | 168878 | 168874 | 36, 37, 29 |
| 2 | GIGYF2 | GRB10 interacting GYF protein 2 | 204120 | 115468 | 115474 | 18, 38, 24 |
| 2 | GAREM2 | GRB2 associated regulator of MAPK1 subtype 2 | 157833 | 084733 | 084754 | 26, 31, 29 |
| 2 | GCC2 | GRIP and coiled-coil domain containing 2 | 135968 | 198075 | 169756 | 22, 25, 4 |
| 2 | GDF7 | growth differentiation factor 7 | 143869 | 118960 | 118961 | 17, 38, |
| 2 | GRB14 | growth factor receptor bound protein 14 | 115290 | 182263 | 082438 | 10, 17, 45 |
| 2 | GREB1 | growth regulation by estrogen in breast cancer 1 | 196208 | 169016 | 169006 | 23, 27, 26 |
| 2 | GULP1 | GULP, engulfment adaptor PTB domain containing 1 | 144366 | 003436 | 174325 | 45, 32, 42 |
| 2 | HS1BP3 | HCLS1 binding protein 3 | 118960 | 143878 | 143869 | 34, 26, 6 |
| 2 | HEATR5B | HEAT repeat containing 5B | 008869 | 115808 | 152133 | 18, 24, 16 |
| 2 | HSPD1 | heat shock protein family D (Hsp60) member 1 | 144381 | 115520 | 115541 | |
| 2 | HSPE1 | heat shock protein family E (Hsp10) member 1 | 115541 | 144381 | 270757 | 7, 33, 29 |
| 2 | HECW2 | HECT, C2 and WW domain containing E3 ubiquitin protein ligase 2 | 138411 | 081320 | 144395 | 45, 25, 36 |
| 2 | HS6ST1 | heparan sulfate 6-O-sulfotransferase 1 | 136720 | 136731 | 222014 | 34, 41, 12 |
| 2 | HES6 | hes family bHLH transcription factor 6 | 144485 | 132323 | 132326 | 3, 35, 27 |
| 2 | HNRNPA3 | heterogeneous nuclear ribonucleoprotein A3 | 170144 | 128654 | 116044 | 18, 11, 2 |
| 2 | HNRNPLL | heterogeneous nuclear ribonucleoprotein L like | 143889 | 119787 | 143891 | 4, 1, 11 |
| 2 | HK2 | hexokinase 2 | 159399 | 135622 | 115350 | |
| 2 | HDLBP | high density lipoprotein binding protein | 115677 | 146205 | 168385 | 43, 30, 45 |
| 2 | HPCAL1 | hippocalcin like 1 | 115756 | 171848 | 115758 | 9, 41, 34 |
| 2 | HNMT | histamine N-methyltransferase | 150540 | 144229 | 144228 | 4, 17, 19 |
| 2 | HAT1 | histone acetyltransferase 1 | 128708 | 115840 | 172878 | 11, 31, 19 |
| 2 | HDAC4 | histone deacetylase 4 | 068024 | 233608 | 280260 | 38, 41, 39 |
| 2 | HJURP | Holliday junction recognition protein | 123485 | 185038 | 144481 | 3, 7, 22 |
| 2 | HOXD1 | homeobox D1 | 128645 | 170166 | 128654 | 17, 4, 35 |
| 2 | HOXD10 | homeobox D10 | 128710 | 128713 | 128709 | 17, 44, 33 |
| 2 | HOXD11 | homeobox D11 | 128713 | 170178 | 128710 | 9, 44, 33 |
| 2 | HOXD12 | homeobox D12 | 170178 | 128714 | 128713 | 23, 17, |
| 2 | HOXD13 | homeobox D13 | 128714 | 174279 | 170178 | 33, 10, 20 |
| 2 | HOXD3 | homeobox D3 | 128652 | 175879 | 278500 | 17, 27, 23 |
| 2 | HOXD4 | homeobox D4 | 170166 | 278500 | 128645 | 17, 36, 44 |
| 2 | HOXD8 | homeobox D8 | 175879 | 128709 | 128652 | 17, 15, 23 |
| 2 | HOXD9 | homeobox D9 | 128709 | 128710 | 175879 | 9, 27, 17 |
| 2 | HSPE1-MOB4 | HSPE1-MOB4 readthrough | 270757 | 115541 | 115540 | |
| 2 | HTRA2 | HtrA serine peptidase 2 | 115317 | 115307 | 115318 | 34, 9, 1 |
| 2 | HADHA | hydroxyacyl-CoA dehydrogenase/3-ketoacyl-CoA thiolase/enoyl-CoA hydratase | 084754 | 157833 | 138029 | 18, 21, 16 |
| | | (trifunctional protein), alpha subunit | | | | |
| 2 | HADHB | hydroxyacyl-CoA dehydrogenase/3-ketoacyl-CoA thiolase/enoyl-CoA hydratase | 138029 | 084754 | 173567 | 15, 2, 13 |
| | | (trifunctional protein), beta subunit | | | | |
| 2 | IKZF2 | IKAROS family zinc finger 2 | 030419 | 178568 | 144451 | 40, 4, 39 |
| 2 | ILKAP | ILK associated serine/threonine phosphatase | 132323 | 178752 | 144485 | 3, 26, 31 |
| 2 | IGKC | Immunoglobulin kappa constant | 211592 | 153574 | 211593 | 30, 41, 18 |
| 2 | IGKJ1 | Immunoglobulin kappa joining 1 | 211597 | 211596 | 211598 | |
| 2 | IGKJ2 | Immunoglobulin kappa joining 2 | 211596 | 211595 | 211597 | |
| 2 | IGKJ3 | Immunoglobulin kappa joining 3 | 211595 | 211594 | 211596 | |
| 2 | IGKJ4 | Immunoglobulin kappa joining 4 | 211594 | 211593 | 211595 | |
| 2 | IGKJ5 | Immunoglobulin kappa joining 5 | 211593 | 211592 | 211594 | |
| 2 | IGKV1OR2- | Immunoglobulin kappa variable 1/OR2-108 (non-functional) | 231292 | 125618 | 136682 | |
| | 108 | | | | | |
| 2 | IGKV1-12 | Immunoglobulin kappa variable 1-12 | 243290 | 241351 | 244437 | |
| 2 | IGKV1-16 | Immunoglobulin kappa variable 1-16 | 240864 | 244437 | 240382 | |
| 2 | IGKV1-17 | Immunoglobulin kappa variable 1-17 | 240382 | 240864 | 239951 | |
| 2 | IGKV1-27 | Immunoglobulin kappa variable 1-27 | 244575 | 241294 | 244116 | |
| 2 | IGKV1-33 | Immunoglobulin kappa variable 1-33 | 242076 | 243238 | 239862 | |
| 2 | IGKV1-37 | Immunoglobulin kappa variable 1-37 (non-functional) | 239862 | 242076 | 242371 | |
| 2 | IGKV1-39 | Immunoglobulin kappa variable 1-39 (gene/pseudogene) | 242371 | 239862 | 273962 | |
| 2 | IGKV1-5 | Immunoglobulin kappa variable 1-5 | 243466 | 211599 | 239855 | |
| 2 | IGKV1-6 | Immunoglobulin kappa variable 1-6 | 239855 | 243466 | 243063 | |
| 2 | IGKV1-8 | Immunoglobulin kappa variable 1-8 | 240671 | 243063 | 241755 | |
| 2 | IGKV1-9 | Immunoglobulin kappa variable 1-9 | 241755 | 240671 | 241351 | |
| 2 | IGKV1D-12 | Immunoglobulin kappa variable 1D-12 | 278857 | 276566 | 211632 | |
| 2 | IGKV1D-13 | Immunoglobulin kappa variable 1D-13 | 276566 | 224041 | 278857 | 22, 20, 16 |
| 2 | IGKV1D-16 | Immunoglobulin kappa variable 1D-16 | 241244 | 242766 | 224041 | |
| 2 | IGKV1D-17 | Immunoglobulin kappa variable 1D-17 | 242766 | 211626 | 241244 | |
| 2 | IGKV1D-33 | Immunoglobulin kappa variable 1D-33 | 239975 | 250036 | 239571 | |
| 2 | IGKV1D-37 | Immunoglobulin kappa variable 1D-37 | 250036 | 251546 | 239975 | |
| 2 | IGKV1D-39 | Immunoglobulin kappa variable 1D-39 | 251546 | 251039 | 250036 | |
| 2 | IGKV1D-42 | Immunoglobulin kappa variable 1D-42 (non-functional) | 211633 | 211632 | 242580 | |
| 2 | IGKV1D-43 | Immunoglobulin kappa variable 1D-43 | 242580 | 211633 | 239819 | |
| 2 | IGKV1D-8 | Immunoglobulin kappa variable 1D-8 | 239819 | 242580 | 228325 | 20, |
| 2 | IGKV2-24 | Immunoglobulin kappa variable 2-24 | 241294 | 211611 | 244575 | |
| 2 | IGKV2-28 | Immunoglobulin kappa variable 2-28 | 244116 | 244575 | 243238 | |
| 2 | IGKV2-30 | Immunoglobulin kappa variable 2-30 | 243238 | 244116 | 242076 | |
| 2 | IGKV2-40 | Immunoglobulin kappa variable 2-40 | 273962 | 242371 | 251039 | |
| 2 | IGKV2D-24 | Immunoglobulin kappa variable 2D-24 (non-functional) | 241566 | 211623 | 225523 | |
| 2 | IGKV2D-26 | Immunoglobulin kappa variable 2D-26 | 211623 | 242534 | 241566 | |
| 2 | IGKV2D-28 | Immunoglobulin kappa variable 2D-28 | 242534 | 243264 | 211623 | |
| 2 | IGKV2D-29 | Immunoglobulin kappa variable 2D-29 | 243264 | 239571 | 242534 | |
| 2 | IGKV2D-30 | Immunoglobulin kappa variable 2D-30 | 239571 | 239975 | 243264 | |
| 2 | IGKV2D-40 | Immunoglobulin kappa variable 2D-40 | 251039 | 273962 | 251546 | |
| 2 | IGKV3OR2- | Immunoglobulin kappa variable 3/OR2-268 (non-functional) | 233999 | 183281 | 125551 | |
| | 268 | | | | | |
| 2 | IGKV3-11 | Immunoglobulin kappa variable 3-11 | 241351 | 241755 | 243290 | |
| 2 | IGKV3-20 | Immunoglobulin kappa variable 3-20 | 239951 | 240382 | 211611 | |
| 2 | IGKV3-7 | Immunoglobulin kappa variable 3-7 (non-functional) | 243063 | 239855 | 240671 | |
| 2 | IGKV3D-11 | Immunoglobulin kappa variable 3D-11 | 211632 | 278857 | 211633 | |
| 2 | IGKV3-15 | Immunoglobulin kappa variable 3D-15 (gene/pseudogene) | 244437 | 243290 | 240864 | |
| 2 | IGKV3D-15 | Immunoglobulin kappa variable 3D-15 (gene/pseudogene) | 224041 | 241244 | 276566 | |
| 2 | IGKV3D-20 | Immunoglobulin kappa variable 3D-20 | 211625 | 225523 | 211626 | |
| 2 | IGKV3D-7 | Immunoglobulin kappa variable 3D-7 | 228325 | 239819 | 259916 | |
| 2 | IGKV4-1 | Immunoglobulin kappa variable 4-1 | 211598 | 211597 | 211599 | |
| 2 | IGKV5-2 | Immunoglobulin kappa variable 5-2 | 211599 | 211598 | 243466 | |
| 2 | IGKV6-21 | Immunoglobulin kappa variable 6-21 (non-functional) | 211611 | 239951 | 241294 | |
| 2 | IGKV6D-21 | Immunoglobulin kappa variable 6D-21 (non-functional) | 225523 | 241566 | 211625 | |
| 2 | IGKV6D-41 | Immunoglobulin kappa variable 6D-41 (non-functional) | 211626 | 211625 | 242766 | |
| 2 | IMP4 | IMP4 homolog, U3 small nucleolar ribonucleoprotein | 136718 | 136710 | 072135 | 41, 21, 36 |
| 2 | IHH | indian hedgehog | 163501 | 181378 | 187736 | 44, 16, 17 |
| 2 | ICOS | inducible T-cell costimulator | 163600 | 163599 | 116117 | 15, 30, 5 |
| 2 | INHA | inhibin alpha subunit | 123999 | 124006 | 144589 | 32, 27, 38 |
| 2 | INHBB | inhibin beta B subunit | 163083 | 281676 | 074047 | 40, 26, 14 |
| 2 | ID2 | inhibitor of DNA binding 2, HLH protein | 115738 | 151692 | 134313 | |
| 2 | ING5 | inhibitor of growth family member 5 | 168395 | 168393 | 180902 | |
| 2 | IMMT | inner membrane mitochondrial protein | 132305 | 132300 | 132313 | 24, 13, 3 |
| 2 | INO80B | INO80 complex subunit B | 115274 | 114993 | 274049 | 39, 14, 6 |
| 2 | INO80D | INO80 complex subunit D | 114933 | 118257 | 023228 | 40, 26, 22 |
| 2 | INO80B- | INO80B-WBP1 readthrough (NMD candidate) | 274049 | 115274 | 239779 | 39, 14, 6 |
| | WBP1 | | | | | |
| 2 | ITPRIPL1 | inositol 1,4,5-trisphosphate receptor interacting protein-like 1 | 198885 | 144028 | 121152 | 38, 15, 14 |
| 2 | INPP1 | inositol polyphosphate-1-phosphatase | 151689 | 198130 | 151690 | 18, 38, 20 |
| 2 | INPP4A | inositol polyphosphate-4-phosphatase type I A | 040933 | 144191 | 183513 | 1, 4, 14 |
| 2 | INPP5D | inositol polyphosphate-5-phosphatase D | 168918 | 115488 | 085978 | 22, 7, 21 |
| 2 | INSIG2 | insulin induced gene 2 | 125629 | 125633 | 163064 | 29, 26, 6 |
| 2 | IGFBP2 | insulin like growth factor binding protein 2 | 115457 | 197756 | 115461 | 27, 44, 12 |
| 2 | IGFBP5 | insulin like growth factor binding protein 5 | 115461 | 115457 | 118245 | 40, 1, 26 |
| 2 | IRS1 | insulin receptor substrate 1 | 169047 | 144460 | 144468 | 10, 40, 26 |
| 2 | ITM2C | integral membrane protein 2C | 135916 | 135932 | 135898 | 31, 8, 26 |
| 2 | ITGA4 | integrin subunit alpha 4 | 115232 | 170035 | 188452 | 39, 45, 22 |
| 2 | ITGA6 | integrin subunit alpha 6 | 091409 | 115844 | 152256 | 13, 4, 43 |
| 2 | ITGAV | integrin subunit alpha V | 138448 | 281192 | 144369 | 29, 4, 12 |
| 2 | ITGB1BP1 | integrin subunit beta 1 binding protein 1 | 119185 | 151693 | 119203 | 22, 43, 29 |
| 2 | ITGB6 | integrin subunit beta 6 | 115221 | 153246 | 153250 | 42, 28, 30 |
| 2 | IFIH1 | interferon induced with helicase C domain 1 | 115267 | 078098 | 115271 | 42, 44, 23 |
| 2 | IL1A | interleukin 1 alpha | 115008 | 169607 | 125538 | 45, 30, 22 |
| 2 | IL1B | interleukin 1 beta | 125538 | 115008 | 125571 | 45, 5, 13 |
| 2 | IL1F10 | interleukin 1 family member 10 (theta) | 136697 | 136695 | 136689 | 32, |
| 2 | IL1RN | interleukin 1 receptor antagonist | 136689 | 136697 | 125637 | 13, 18, 5 |
| 2 | IL1RL1 | interleukin 1 receptor like 1 | 115602 | 115598 | 115604 | 32, 45, 1 |
| 2 | IL1RL2 | interleukin 1 receptor like 2 | 115598 | 115594 | 115602 | 42, 23, 35 |
| 2 | IL1R1 | interleukin 1 receptor type 1 | 115594 | 115590 | 115598 | 29, 37, 45 |
| 2 | IL1R2 | interleukin 1 receptor type 2 | 115590 | 071054 | 115594 | 18, 3, 30 |
| 2 | IL18R1 | interleukin 18 receptor 1 | 115604 | 115602 | 115607 | 27, 19, 23 |
| 2 | IL18RAP | interleukin 18 receptor accessory protein | 115607 | 115604 | 180251 | 5, 45, 36 |
| 2 | IL36RN | interleukin 36 receptor antagonist | 136695 | 136696 | 136697 | |
| 2 | IL36A | interleukin 36, alpha | 136694 | 136688 | 136696 | 13, |
| 2 | IL36B | interleukin 36, beta | 136696 | 136694 | 136695 | 10, 8, |
| 2 | IL36G | interleukin 36, gamma | 136688 | 125571 | 136694 | 24, 30, 16 |
| 2 | IL37 | interleukin 37 | 125571 | 125538 | 136688 | 32, 16, |
| 2 | ITSN2 | intersectin 2 | 198399 | 186453 | 084676 | 22, 40, 35 |
| 2 | IFT172 | intraflagellar transport 172 | 138002 | 157992 | 115226 | 41, 9, 24 |
| 2 | IQCA1 | IQ motif containing with AAA domain 1 | 132321 | 182177 | 144476 | 31, 40, 30 |
| 2 | ICA1L | islet cell autoantigen 1 like | 163596 | 138439 | 138442 | 11, 28, 40 |
| 2 | IAH1 | isoamyl acetate-hydrolyzing esterase 1 homolog | 134330 | 119203 | 151694 | 18, 29, 38 |
| 2 | IDH1 | isocitrate dehydrogenase (NADP(+)) 1, cytosolic | 138413 | 188674 | 115020 | 19, 4, 2 |
| 2 | IWS1 | IWS1, SUPT6H interacting protein | 163166 | 115718 | 169994 | 13, 24, 39 |
| 2 | KANSL1L | KAT8 regulatory NSL complex subunit 1 like | 144445 | 197713 | 115361 | 26, 22, 29 |
| 2 | KANSL3 | KAT8 regulatory NSL complex subunit 3 | 114982 | 196843 | 249715 | 40, 21, 38 |
| 2 | KLHL23 | kelch like family member 23 | 213160 | 144362 | 138385 | 4, 2, 13 |
| 2 | KLHL29 | kelch like family member 29 | 119771 | 218819 | 119778 | 26, 36, 22 |
| 2 | KLHL30 | kelch like family member 30 | 168427 | 144488 | 178752 | 25, 24, 28 |
| 2 | KLHL41 | kelch like family member 41 | 239474 | 251569 | 138399 | 25, 24, 15 |
| 2 | KRTCAP3 | keratinocyte associated protein 3 | 157992 | 115216 | 138002 | 3, 18, 28 |
| 2 | KHK | ketohexokinase | 138030 | 138080 | 138028 | 19, 3, 17 |
| 2 | KIAA1211L | KIAA1211 like | 196872 | 071073 | 135951 | 27, 43, 29 |
| 2 | KIAA1841 | KIAA1841 | 162929 | 162928 | 237651 | 26, 11, 9 |
| 2 | KIAA2012 | KIAA2012 | 182329 | 155760 | 283310 | |
| 2 | KIAA2012 | KIAA2012 | 283310 | 182329 | 116030 | |
| 2 | KIDINS220 | kinase D interacting substrate 220 | 134313 | 115738 | 143797 | 18, 11, 31 |
| 2 | KIF1A | kinesin family member 1A | 130294 | 184945 | 172482 | |
| 2 | KIF3C | kinesin family member 3C | 084731 | 143970 | 084733 | 29, 26, 34 |
| 2 | KIF5C | kinesin family member 5C | 168280 | 135999 | 150556 | 12, 28, 8 |
| 2 | KLF11 | Kruppel like factor 11 | 172059 | 134317 | 205795 | 40, 27, 9 |
| 2 | KLF7 | Kruppel like factor 7 | 118263 | 144410 | 118260 | 26, 43, 18 |
| 2 | KYNU | kynureninase | 115919 | 168702 | 075884 | 30, 9, 11 |
| 2 | LCT | lactase | 115850 | 144224 | 076003 | 39, 16, 27 |
| 2 | LBX2 | ladybird homeobox 2 | 179528 | 115282 | 115289 | 34, 19, 14 |
| 2 | LANCL1 | LanC like 1 | 115365 | 168530 | 021826 | 8, 29, 2 |
| 2 | LTBP1 | latent transforming growth factor beta binding protein 1 | 049323 | 018699 | 152689 | 45, 6, 15 |
| 2 | LRP1B | LDL receptor related protein 1B | 168702 | 144227 | 115919 | 25, 8, 24 |
| 2 | LRP2 | LDL receptor related protein 2 | 081479 | 073737 | 163093 | 17, 40, 32 |
| 2 | LMAN2L | lectin, mannose binding 2 like | 114988 | 249715 | 158158 | 18, 1, 36 |
| 2 | LRPPRC | leucine rich pentatricopeptide repeat containing | 138095 | 143921 | 138032 | 42, 3, 38 |
| 2 | LRRTM1 | leucine rich repeat transmembrane neuronal 1 | 162951 | 066032 | 163541 | 8, 40, 28 |
| 2 | LRRTM4 | leucine rich repeat transmembrane neuronal 4 | 176204 | 005436 | 143954 | 9, 8, 38 |
| 2 | LIMS1 | LIM zinc finger domain containing 1 | 169756 | 135968 | 153201 | 7, 4, 13 |
| 2 | LIMS2 | LIM zinc finger domain containing 2 | 072163 | 169994 | 144230 | 26, 36, 15 |
| 2 | LIMS3 | LIM zinc finger domain containing 3 | 256977 | 015568 | 144063 | 7, 24, 38 |
| 2 | LIMS4 | LIM zinc finger domain containing 4 | 256671 | 257207 | 183054 | |
| 2 | LBH | limb bud and heart development | 213626 | 119801 | 172954 | 39, 41, 36 |
| 2 | LDAH | lipid droplet associated hydrolase | 118961 | 143869 | 084674 | 29, 6, 24 |
| 2 | LPIN1 | lipin 1 | 134324 | 169006 | 071575 | 2, 7, 38 |
| 2 | LIPT1 | lipoyltransferase 1 | 144182 | 273045 | 273155 | 34, 30, 22 |
| 2 | LONRF2 | LON peptidase N-terminal domain and ring finger 2 | 170500 | 144218 | 115526 | 2, 26, 31 |
| 2 | LINC01125 | Long intergenic non-protein coding RNA 1125 | 228486 | 115073 | 115085 | 1, 38, 42 |
| 2 | LINC00116 | Long intergenic non-protein coding RNA 116 | 175701 | 144061 | 257207 | 25, 31, 33 |
| 2 | LRRFIP1 | LRR binding FLII interacting protein 1 | 124831 | 124839 | 177483 | 29, 22, 6 |
| 2 | LNPK | lunapark, ER junction formation factor | 144320 | 154518 | 174279 | 29, 22, 42 |
| 2 | LHCGR | luteinizing hormone/choriogonadotropin receptor | 138039 | 279956 | 170820 | 4, 5, 38 |
| 2 | LYPD1 | LY6/PLAUR domain containing 1 | 150551 | 183840 | 176771 | 31, 27, 4 |
| 2 | LYPD6 | LY6/PLAUR domain containing 6 | 187123 | 150556 | 168288 | 4, 11, 31 |
| 2 | LYPD6B | LY6/PLAUR domain containing 6B | 150556 | 168280 | 187123 | |
| 2 | LY75-CD302 | LY75-CD302 readthrough | 248672 | 241399 | 054219 | |
| 2 | LY75 | lymphocyte antigen 75 | 054219 | 248672 | 153246 | 42, 22, 39 |
| 2 | KDM3A | lysine demethylase 3A | 115548 | 068615 | 115561 | 18, 29, 42 |
| 2 | KRCC1 | lysine rich coiled-coil 1 | 172086 | 185304 | 115593 | 28, 22, 6 |
| 2 | LCLAT1 | lysocardiolipin acyltransferase 1 | 172954 | 213626 | 162949 | 11, 45, 24 |
| 2 | LAPTM4A | lysosomal protein transmembrane 4 alpha | 068697 | 132031 | 115884 | 31, 13, 11 |
| 2 | LYG1 | lysozyme g1 | 144214 | 185674 | 115514 | 10, 27, 17 |
| 2 | LYG2 | lysozyme g2 | 185674 | 185414 | 144214 | 14, 35, 38 |
| 2 | LOXL3 | lysyl oxidase like 3 | 115318 | 115317 | 115325 | 6, 34, 43 |
| 2 | MARCO | macrophage receptor with collagenous structure | 019169 | 163064 | 144119 | 10, 1, 20 |
| 2 | MROH2A | maestro heat like repeat family member 2A | 185038 | 241635 | 123485 | |
| 2 | MFSD2B | major facilitator superfamily domain containing 2B | 205639 | 173960 | 163026 | 12, 21, 32 |
| 2 | MFSD6 | major facilitator superfamily domain containing 6 | 151690 | 151689 | 189362 | 26, 27, 18 |
| 2 | MFSD9 | major facilitator superfamily domain containing 9 | 135953 | 115616 | 170417 | 37, 27, 16 |
| 2 | MAL | mal, T-cell differentiation protein | 172005 | 163060 | 144029 | 30, 8, 11 |
| 2 | MALL | mal, T-cell differentiation protein like | 144063 | 256977 | 144061 | 37, 24, 40 |
| 2 | MDH1 | malate dehydrogenase 1 | 014641 | 143951 | 281071 | 29, 30, 40 |
| 2 | MDH1B | malate dehydrogenase 1B | 138400 | 232125 | 118246 | 38, 11, 42 |
| 2 | MGAT4A | mannosyl (alpha-1,3-)-glycoprotein beta-1,4-N-acetylglucosaminyltransferase, | 071073 | 115446 | 196872 | 29, 39, 31 |
| | | isozyme A | | | | |
| 2 | MGAT5 | mannosyl (alpha-1,6-)-glycoprotein beta-1,6-N-acetyl-glucosaminyltransferase | 152127 | 176771 | 152128 | 22, 41, 16 |
| 2 | MOGS | mannosyl-oligosaccharide glucosidase | 115275 | 239779 | 204822 | 27, 38, 5 |
| 2 | MATN3 | matrilin 3 | 132031 | 118965 | 068697 | 6, 26, 11 |
| 2 | MAIP1 | matrix AAA peptidase interacting protein 1 | 162972 | 162971 | 196141 | 10, 39, 33 |
| 2 | MXD1 | MAX dimerization protein 1 | 059728 | 124380 | 279229 | |
| 2 | MEMO1 | mediator of cell motility 1 | 162959 | 277893 | 162961 | 10, 3, 35 |
| 2 | M1AP | meiosis 1 associated protein | 159374 | 115325 | 135622 | 38, 44, 32 |
| 2 | MEIS1 | Meis homeobox 1 | 143995 | 198369 | 143971 | 18, 44, 42 |
| 2 | MLPH | melanophilin | 115648 | 163359 | 071677 | 23, 42, 3 |
| 2 | MREG | melanoregulin | 118242 | 115414 | 115425 | 10, 35, 34 |
| 2 | MARCH4 | membrane associated ring-CH-type finger 4 | 144583 | 079246 | 138375 | 35, 12, 20 |
| 2 | MARCH7 | membrane associated ring-CH-type finger 7 | 136536 | 123636 | 241399 | 40, 13, 45 |
| 2 | MBOAT2 | membrane bound O-acyltransferase domain containing 2 | 143797 | 134313 | 151693 | |
| 2 | MPP4 | membrane palmitoylated protein 4 | 082126 | 155755 | 003393 | 14, 25, 10 |
| 2 | MERTK | MER proto-oncogene, tyrosine kinase | 153208 | 153107 | 153214 | 30, 25, 2 |
| 2 | MSGN1 | mesogenin 1 | 151379 | 178295 | 170745 | |
| 2 | MTA3 | metastasis associated 1 family member 3 | 057935 | 171126 | 162881 | 18, 10, 12 |
| 2 | MTX2 | metaxin 2 | 128654 | 128645 | 170144 | 38, 25, 11 |
| 2 | MAT2A | methionine adenosyltransferase 2A | 168906 | 152292 | 115486 | |
| 2 | METAP1D | methionyl aminopeptidase type 1D, mitochondrial | 172878 | 128708 | 144355 | 26, 34, 2 |
| 2 | MARS2 | methionyl-tRNA synthetase 2, mitochondrial | 247626 | 162944 | 152430 | 39, 42, 23 |
| 2 | MBD5 | methyl-CpG binding domain protein 5 | 204406 | 115947 | 135999 | 31, 2, 40 |
| 2 | MTHFD2 | methylenetetrahydrofolate dehydrogenase (NADP+ dependent) 2, | 065911 | 114978 | 264324 | 13, 5, 34 |
| | | methenyltetrahydrofolate cyclohydrolase | | | | |
| 2 | MMADHC | methylmalonic aciduria and homocystinuria, cbID type | 168288 | 187123 | 115963 | 4, 7, 13 |
| 2 | MCEE | methylmalonyl-CoA epimerase | 124370 | 124357 | 124383 | 11, 19, 22 |
| 2 | METTL21A | methyltransferase like 21A | 144401 | 118260 | 163249 | 21, 3, 28 |
| 2 | METTL5 | methyltransferase like 5 | 138382 | 138385 | 144357 | 34, 22, 33 |
| 2 | METTL8 | methyltransferase like 8 | 123600 | 198586 | 115827 | 13, 6, 15 |
| 2 | MAP2 | microtubule associated protein 2 | 078018 | 144407 | 144406 | 8, 25, 31 |
| 2 | MAPRE3 | microtubule associated protein RP/EB family member 3 | 084764 | 157851 | 119777 | 28, 29, 14 |
| 2 | MITD1 | microtubule interacting and trafficking domain containing 1 | 158411 | 273155 | 185414 | 11, 22, 29 |
| 2 | MCM6 | minichromosome maintenance complex component 6 | 076003 | 115850 | 115866 | 39, 34, 24 |
| 2 | MFF | mitochondrial fission factor | 168958 | 169031 | 168955 | 38, 2, 6 |
| 2 | MRPL19 | mitochondrial ribosomal protein L19 | 115364 | 115363 | 005436 | |
| 2 | MRPL30 | mitochondrial ribosomal protein L30 | 185414 | 158411 | 185674 | 7, 42, 36 |
| 2 | MRPL33 | mitochondrial ribosomal protein L33 | 243147 | 163798 | 171174 | 25, 31, 7 |
| 2 | MRPL35 | mitochondrial ribosomal protein L35 | 132313 | 132305 | 068615 | 3, 42, 24 |
| 2 | MRPL44 | mitochondrial ribosomal protein L44 | 135900 | 085449 | 135919 | 15, 21, 12 |
| 2 | MRPL53 | mitochondrial ribosomal protein L53 | 204822 | 115275 | 135637 | 29, 15, 1 |
| 2 | MRPS5 | mitochondrial ribosomal protein S5 | 144029 | 172005 | 144026 | 24, 3, 43 |
| 2 | MRPS9 | mitochondrial ribosomal protein S9 | 135972 | 198914 | 135973 | 43, 31, 3 |
| 2 | MTERF4 | mitochondrial transcription termination factor 4 | 122085 | 162804 | 115687 | 15, 39, 30 |
| 2 | MTIF2 | mitochondrial translational initiation factor 2 | 085760 | 143947 | 115355 | 7, 19, 6 |
| 2 | MAP3K19 | mitogen-activated protein kinase kinase kinase 19 | 176601 | 082258 | 115839 | |
| 2 | MAP3K2 | mitogen-activated protein kinase kinase kinase 2 | 169967 | 163161 | 115718 | 22, 13, 42 |
| 2 | MAP4K3 | mitogen-activated protein kinase kinase kinase kinase 3 | 011566 | 205111 | 152154 | 4, 11, 2 |
| 2 | MAP4K4 | mitogen-activated protein kinase kinase kinase kinase 4 | 071054 | 196460 | 115590 | 6, 22, 24 |
| 2 | MZT2A | mitotic spindle organizing protein 2A | 173272 | 233087 | 075886 | 3, 21, 28 |
| 2 | MZT2B | mitotic spindle organizing protein 2B | 152082 | 136699 | 152086 | 33, 26, 15 |
| 2 | MOB4 | MOB family member 4, phocein | 115540 | 270757 | 162944 | 2, 22, 17 |
| 2 | MOB1A | MOB kinase activator 1A | 114978 | 163170 | 065911 | 22, 1, 13 |
| 2 | MOGAT1 | monoacylglycerol O-acyltransferase 1 | 124003 | 116120 | 123983 | |
| 2 | MORN2 | MORN repeat containing 2 | 188010 | 163214 | 214694 | 29, 17, 1 |
| 2 | MPHOSPH10 | M-phase phosphoprotein 10 | 124383 | 124370 | 124374 | 13, 3, 11 |
| 2 | MPV17 | MPV17, mitochondrial inner membrane protein | 115204 | 163794 | 115207 | 40, 23, 18 |
| 2 | MCFD2 | multiple coagulation factor deficiency 2 | 180398 | 171150 | 068724 | 42, 29, 45 |
| 2 | MSH2 | mutS homolog 2 | 095002 | 119888 | 184261 | 39, 12, 38 |
| 2 | MSH6 | mutS homolog 6 | 116062 | 184261 | 138081 | 12, 14, 38 |
| 2 | MYTH | myelin transcription factor 1 like | 186487 | 130508 | 032389 | |
| 2 | MYO1B | myosin IB | 128641 | 138378 | 173559 | 24, 11, 10 |
| 2 | MYO3B | myosin IIIB | 071909 | 144357 | 204335 | 31, 23, 24 |
| 2 | MYL1 | myosin light chain 1 | 168530 | 279317 | 115365 | 25, 15, 30 |
| 2 | MYO7B | myosin VIIB | 169994 | 163166 | 072163 | 5, 36, 26 |
| 2 | MSTN | myostatin | 138379 | 187699 | 198130 | 20, 15, 25 |
| 2 | NAGK | N-acetylglucosamine kinase | 124357 | 144043 | 124370 | 36, 30, 39 |
| 2 | NAT8 | N-acetyltransferase 8 (putative) | 144035 | 116127 | 204872 | 17, 19, 36 |
| 2 | NAT8B | N-acetyltransferase 8B (putative, gene/pseudogene) | 204872 | 144035 | 144034 | 27, 16, |
| 2 | NDUFAF7 | NADH: ubiquinone oxidoreductase complex assembly factor 7 | 003509 | 115816 | 115825 | 36, 38, 30 |
| 2 | NDUFS1 | NADH: ubiquinone oxidoreductase core subunit S1 | 023228 | 114933 | 114942 | 24, 45, 15 |
| 2 | NDUFA10 | NADH: ubiquinone oxidoreductase subunit A10 | 130414 | 280260 | 182083 | |
| 2 | NDUFB3 | NADH: ubiquinone oxidoreductase subunit B3 | 119013 | 155744 | 003402 | 29, 15, 25 |
| 2 | NPPC | natriuretic peptide C | 163273 | 144524 | 144535 | |
| 2 | NCK2 | NCK adaptor protein 2 | 071051 | 115641 | 119147 | 35, 34, 32 |
| 2 | NCKAP1 | NCK associated protein 1 | 061676 | 162998 | 162999 | 24, 45, 42 |
| 2 | NCKAP5 | NCK associated protein 5 | 176771 | 150551 | 152127 | 11, 43, 18 |
| 2 | NEB | nebulin | 183091 | 080345 | 162980 | 25, 18, 24 |
| 2 | NPHP1 | nephrocystin 1 | 144061 | 144063 | 175701 | 31, 29, 43 |
| 2 | NEURL3 | neuralized E3 ubiquitin protein ligase 3 | 163121 | 121152 | 196843 | 4, 39, 28 |
| 2 | NEU2 | neuraminidase 2 | 115488 | 066248 | 168918 | |
| 2 | NEU4 | neuraminidase 4 | 204099 | 154252 | 188389 | 8, 19, 16 |
| 2 | NRXN1 | neurexin 1 | 179915 | 170820 | 115239 | 38, |
| 2 | NXPH2 | neurexophilin 2 | 144227 | 144228 | 168702 | 17, 14, 12 |
| 2 | NBEAL1 | neurobeachin like 1 | 144426 | 138380 | 119004 | 23, 16, |
| 2 | NBAS | neuroblastoma amplified sequence | 151779 | 162981 | 079785 | 10, 14, 23 |
| 2 | NMS | neuromedin S | 204640 | 115526 | 115539 | |
| 2 | NMUR1 | neuromedin U receptor 1 | 171596 | 115053 | 177673 | 36, 1, 26 |
| 2 | NEUROD1 | neuronal differentiation 1 | 162992 | 188452 | 138434 | 28, 14, 25 |
| 2 | NGEF | neuronal guanine nucleotide exchange factor | 066248 | 182600 | 115488 | 2, 6, 26 |
| 2 | NPAS2 | neuronal PAS domain protein 2 | 170485 | 115539 | 071082 | 35, 20, 16 |
| 2 | NYAP2 | neuronal tyrosine-phosphorylated phosphoinositide-3-kinase adaptor 2 | 144460 | 135905 | 169047 | 45, 8, |
| 2 | NRP2 | neuropilin 2 | 118257 | 116117 | 114933 | 27, 43, 15 |
| 2 | NTSR2 | neurotensin receptor 2 | 169006 | 196208 | 134324 | 8, 38, 10 |
| 2 | NFU1 | NFU1 iron-sulfur cluster scaffold | 169599 | 198380 | 115977 | 24, 1, 28 |
| 2 | NAB1 | NGFI-A binding protein 1 | 138386 | 189362 | 115419 | |
| 2 | NIF3L1 | NGG1 interacting factor 3 like 1 | 196290 | 240344 | 115942 | 41, 27, 30 |
| 2 | NOSTRIN | nitric oxide synthase trafficking | 163072 | 172292 | 152253 | 7, 34, 16 |
| 2 | NLRC4 | NLR family CARD domain containing 4 | 091106 | 152683 | 119820 | 5, 4, 39 |
| 2 | NMI | N-myc and STAT interactor | 123609 | 184898 | 123610 | 34, 43, 11 |
| 2 | NHEJ1 | non-homologous end joining factor 1 | 187736 | 163501 | 280537 | 4, 40, 9 |
| 2 | NCAPH | non-SMC condensin I complex subunit H | 121152 | 198885 | 163121 | 28, 38, 22 |
| 2 | NOP58 | NOP58 ribonucleoprotein | 055044 | 116030 | 204217 | 41, 7, 6 |
| 2 | NOTO | notochord homeobox | 214513 | 135631 | 135632 | 38, |
| 2 | NT5C1B- | NT5C1B-RDH14readthrough | 250741 | 240857 | 185013 | |
| | RDH14 | | | | | |
| 2 | NEMP2 | nuclear envelope integral membrane protein 2 | 189362 | 151690 | 138386 | |
| 2 | NFE2L2 | nuclear factor, erythroid 2 like 2 | 116044 | 170144 | 018510 | 24, 45, 42 |
| 2 | NRBP1 | nuclear receptor binding protein 1 | 115216 | 115241 | 157992 | |
| 2 | NCOA1 | nuclear receptor coactivator 1 | 084676 | 198399 | 184924 | |
| 2 | NR4A2 | nuclear receptor subfamily 4 group A member 2 | 153234 | 162989 | 115159 | 42, 26, 24 |
| 2 | NABP1 | nucleic acid binding protein 1 | 173559 | 128641 | 168497 | 1, 27, 13 |
| 2 | NOL10 | nucleolar protein 10 | 115761 | 115758 | 143882 | 30, 34, 13 |
| 2 | NIFK | nucleolar protein interacting with the FHA domain of MKI67 | 155438 | 074054 | 211460 | 7, 4, 34 |
| 2 | NCL | nucleolin | 115053 | 156966 | 171596 | 3, 35, 18 |
| 2 | NUP35 | nudeoporin 35 | 163002 | 162999 | 170396 | 4, 34, 26 |
| 2 | OLA1 | Obg like ATPase 1 | 138430 | 172845 | 217236 | 4, 7, 38 |
| 2 | OBSL1 | obscurin like 1 | 124006 | 188760 | 123999 | 27, 40, 1 |
| 2 | OSR1 | odd-skipped related transciption factor 1 | 143867 | 185013 | 183891 | 1, 38, 15 |
| 2 | OR6B2 | olfactory receptor family 6 subfamily B member 2 | 182083 | 130414 | 221961 | |
| 2 | OR6B3 | olfactory receptor family 6 subfamily B member 3 | 178586 | 221961 | 172428 | |
| 2 | OST4 | oligosaccharyltransferase complex subunit 4, non-catalytic | 228474 | 084693 | 138080 | 34, 7, 43 |
| 2 | ORC2 | origin recognition complex subunit 2 | 115942 | 196290 | 155744 | 26, 13, 29 |
| 2 | ORC4 | origin recognition complex subunit 4 | 115947 | 121989 | 204406 | 26, 2, 30 |
| 2 | ORMDL1 | ORMDL sphingolipid biosynthesis regulator 1 | 128699 | 128694 | 064933 | 22, 11, 36 |
| 2 | ODC1 | ornithine decarboxylase 1 | 115758 | 115756 | 115761 | 21, 41, 18 |
| 2 | OTX1 | orthodenticle homeobox 1 | 115507 | 115504 | 143951 | 13, 23, 14 |
| 2 | OSGEPL1 | O-sialoglycoprotein endopeptidase like 1 | 128694 | 151687 | 128699 | 30, 34, 40 |
| 2 | OTOF | otoferlin | 115155 | 157856 | 173557 | 39, 6, 17 |
| 2 | OTOS | otospiralin | 178602 | 172428 | 063660 | 31, 40, 8 |
| 2 | OXER1 | oxoeicosanoid receptor 1 | 162881 | 057935 | 162882 | 39, 35, 25 |
| 2 | OSBPL6 | oxysterol binding protein like 6 | 079156 | 155636 | 180228 | 42, 6, 18 |
| 2 | PAX3 | paired box 3 | 135903 | 116106 | 163081 | 18, 35, 38 |
| 2 | PAX8 | paired box 8 | 125618 | 125637 | 231292 | 40, 27, 17 |
| 2 | PARD3B | par-3 family cell polarity regulator beta | 116117 | 163600 | 118257 | 11, 1, 27 |
| 2 | PTH2R | parathyroid hormone 2 receptor | 144407 | 115020 | 078018 | |
| 2 | PNKD | paroxysmal nonkinesigenic dyskinesia | 127838 | 127837 | 135926 | 29, 15, 31 |
| 2 | PNO1 | partner of NOB1 homolog | 115946 | 273398 | 221823 | 29, 4, 2 |
| 2 | PASK | PAS domain containing serine/threonine kinase | 115687 | 122085 | 115685 | 22, 39, 21 |
| 2 | PELI1 | pellino E3 ubiquitin protein ligase 1 | 197329 | 143952 | 119862 | 42, 22, 26 |
| 2 | PTCD3 | pentatricopeptide repeat domain 3 | 132300 | 068654 | 132305 | 39, 24, 3 |
| 2 | PPIG | peptidylprolyl isomerase G | 138398 | 138399 | 154479 | 29, 6, 7 |
| 2 | PPIL3 | peptidylprolyl isomerase like 3 | 240344 | 013441 | 196290 | 41, 4, 28 |
| 2 | PTRHD1 | peptidyl-tRNA hydrolase domain containing 1 | 184924 | 084676 | 138092 | 28, 2, 38 |
| 2 | PER2 | period circadian clock 2 | 132326 | 144485 | 204104 | 18, 45, 1 |
| 2 | PXDN | peroxidasin | 130508 | 115705 | 186487 | 43, 9, 15 |
| 2 | PEX13 | peroxisomal biogenesis factor 13 | 162928 | 162927 | 162929 | 13, 31, 43 |
| 2 | PECR | peroxisomal trans-2-enoyl-CoA reductase | 115425 | 118242 | 163449 | 7, 19, 25 |
| 2 | FARSB | phenylalanyl-tRNA synthetase beta subunit | 116120 | 163082 | 124003 | 10, 7, 13 |
| 2 | PDCL3 | phosducin like 3 | 115539 | 204640 | 170485 | 1, 30, 15 |
| 2 | PHOSPHO2 | phosphatase, orphan 2 | 144362 | 154479 | 213160 | 38, 4, 37 |
| 2 | PIGF | phosphatidylinositol glycan anchor biosynthesis class F | 151665 | 119729 | 119878 | 29, 11, 31 |
| 2 | PDE11A | phosphodiesterase 11A | 128655 | 197557 | 155636 | 18, 26, 29 |
| 2 | PDE1A | phosphodiesterase 1A | 115252 | 150722 | 077232 | 8, 40, 38 |
| 2 | PDE6D | phosphodiesterase 6D | 156973 | 187514 | 144524 | 21, 35, 5 |
| 2 | PIKFYVE | phosphoinositide kinase, FYVE-type zinc finger containing | 115020 | 138413 | 144407 | 22, 4, 5 |
| 2 | PLA2R1 | phospholipase A2 receptor 1 | 153246 | 054219 | 115221 | 29, 18, 42 |
| 2 | PLB1 | phospholipase B1 | 163803 | 075426 | 213639 | 7, 5, 2 |
| 2 | PLCD4 | phospholipase C delta 4 | 115556 | 144580 | 115568 | 25, 24, 40 |
| 2 | PLCL1 | phospholipase C like 1 | 115896 | 152430 | 119042 | 15, 17, 44 |
| 2 | PID1 | phosphotyrosine interaction domain containing 1 | 153823 | 153820 | 187957 | 15, 2, 36 |
| 2 | PKP4 | plakophilin 4 | 144283 | 153237 | 281472 | 11, 18, 40 |
| 2 | PLGLB1 | plasminogen-like B1 | 183281 | 187627 | 233999 | 19, 22, 20 |
| 2 | PLGLB2 | plasminogen-like B2 | 125551 | 233999 | 185304 | 19, 13, 25 |
| 2 | PLEK | pleckstrin | 115956 | 119865 | 204923 | 21, 30, 5 |
| 2 | PSD4 | pleckstrin and Sec7 domain containing 4 | 125637 | 136689 | 125618 | 21, 39, 22 |
| 2 | PLEKHA3 | pleckstrin homology domain containing A3 | 116095 | 079150 | 155657 | 20, 29, 1 |
| 2 | PLEKHB2 | pleckstrin homology domain containing B2 | 115762 | 152102 | 188219 | 8, 2, 37 |
| 2 | PLEKHM3 | pleckstrin homology domain containing M3 | 178385 | 163251 | 118231 | |
| 2 | PLEKHH2 | pleckstrin homology, MyTH4 and FERM domain containing H2 | 152527 | 115970 | 223658 | 42, 44, 20 |
| 2 | PMS1 | PMS1 homolog 1, mismatch repair system component | 064933 | 128699 | 187699 | 10, 22, 38 |
| 2 | PAIP2B | poly(A) binding protein interacting protein 2B | 124374 | 124383 | 075292 | 27, 15, 8 |
| 2 | PAPOLG | poly(A) polymerase gamma | 115421 | 119866 | 162924 | 24, 22, 38 |
| 2 | PCBP1 | poly(rC) binding protein 1 | 169564 | 244617 | 115998 | 1, 43, 27 |
| 2 | PCGF1 | polycomb group ring finger 1 | 115289 | 179528 | 115297 | 40, 3, 15 |
| 2 | GALNT13 | polypeptide N-acetylgalactosaminyltransferase 13 | 144278 | 177519 | 162989 | 18, 10, 30 |
| 2 | GALNT14 | polypeptide N-acetylgalactosaminyltransferase 14 | 158089 | 162949 | 214711 | |
| 2 | GALNT3 | polypeptide N-acetylgalactosaminyltransferase 3 | 115339 | 178662 | 123607 | 25, 16, 38 |
| 2 | GALNT5 | polypeptide N-acetylgalactosaminyltransferase 5 | 136542 | 115159 | 136541 | 20, |
| 2 | PNPT1 | polyribonucleotide nucleotidyltransferase 1 | 138035 | 275052 | 115380 | 1, 26, 4 |
| 2 | PGAP1 | post-GPI attachment to proteins 1 | 197121 | 187944 | 065413 | 11, 2, 26 |
| 2 | KCMF1 | potassium channel modulatory factor 1 | 176407 | 034510 | 152284 | 43, 22, 40 |
| 2 | KCTD18 | potassium channel tetramerization domain containing 18 | 155729 | 196141 | 163535 | 43, 22, 30 |
| 2 | KCNK12 | potassium two pore domain channel subfamily K member 12 | 184261 | 095002 | 116062 | 21, 41, 26 |
| 2 | KCNK3 | potassium two pore domain channel subfamily K member 3 | 171303 | 157884 | 213699 | 2, 28, 32 |
| 2 | KCNIP3 | potassium voltage-gated channel interacting protein 3 | 115041 | 155066 | 115042 | 8, 29, 24 |
| 2 | KCNF1 | potassium voltage-gated channel modifier subfamily F member 1 | 162975 | 143870 | 150873 | 8, 28, 16 |
| 2 | KCNG3 | potassium voltage-gated channel modifier subfamily G member 3 | 171126 | 115944 | 057935 | 12, 44, 38 |
| 2 | KCNS3 | potassium voltage-gated channel modifier subfamily S member 3 | 170745 | 151379 | 240857 | 15, 37, 23 |
| 2 | KCNE4 | potassium voltage-gated channel subfamily E regulatory subunit 4 | 152049 | 123983 | 171951 | 43, 26, 20 |
| 2 | KCNH7 | potassium voltage-gated channel subfamily H member 7 | 184611 | 115271 | 182263 | 45, 10, 28 |
| 2 | KCNJ13 | potassium voltage-gated channel subfamily J member 13 | 115474 | 204120 | 182600 | 29, 35, 14 |
| 2 | KCNJ3 | potassium voltage-gated channel subfamily J member 3 | 162989 | 144278 | 153234 | 4, 25, 15 |
| 2 | POTEE | POTE ankyrin domain family member E | 188219 | 115762 | 233087 | 33, 38, |
| 2 | POTEF | POTE ankyrin domain family member F | 196604 | 222014 | 152076 | 15, 23, 38 |
| 2 | POTEI | POTE ankyrin domain family member I | 196834 | 072135 | 152093 | 33, 23, 38 |
| 2 | POTEJ | POTE ankyrin domain family member J | 222038 | 136698 | 173302 | 33, |
| 2 | POU3F3 | POU class 3 homeobox 3 | 198914 | 170417 | 135972 | 12, 38, |
| 2 | PQLC3 | PQ loop repeat containing 3 | 162976 | 150873 | 134318 | 29, 1, 17 |
| 2 | PRPF40A | pre-mRNA processing factor 40 homolog A | 196504 | 157827 | 177917 | 19, 43, 29 |
| 2 | PCYOX1 | prenylcysteine oxidase 1 | 116005 | 116001 | 143977 | 42, 29, 11 |
| 2 | PFN4 | profilin family member 4 | 176732 | 115129 | 276087 | 38, 22, 33 |
| 2 | PDCD1 | programmed cell death 1 | 188389 | 204099 | 188011 | 5, 17, 23 |
| 2 | PROKR1 | prokineticin receptor 1 | 169618 | 169621 | 163219 | 38, |
| 2 | PREB | prolactin regulatory element binding | 138073 | 143994 | 186143 | 3, 34, 21 |
| 2 | PRLH | prolactin releasing hormone | 071677 | 115648 | 124839 | |
| 2 | PRR21 | proline rich 21 | 221961 | 182083 | 178586 | |
| 2 | PRR30 | proline rich 30 | 186143 | 138073 | 163792 | 38, 30, 8 |
| 2 | PREPL | prolyl endopeptidase-like | 138078 | 138079 | 143919 | 8, 42, 26 |
| 2 | PROM2 | prominin 2 | 155066 | 233757 | 115041 | 13, 18, 24 |
| 2 | POMC | proopiomelanocortin | 115138 | 084710 | 119772 | 31, 38, 8 |
| 2 | PRADC1 | protease associated domain containing 1 | 135617 | 135632 | 135624 | 21, 25, 14 |
| 2 | PRSS56 | protease, serine 56 | 237412 | 171551 | 135902 | 38, 14, 12 |
| 2 | PSMD1 | proteasome 26S subunit, non-ATPase 1 | 173692 | 204128 | 135914 | |
| 2 | PSMD14 | proteasome 26S subunit, non-ATPase 14 | 115233 | 136560 | 136535 | 10, 30, 7 |
| 2 | PSME4 | proteasome activator subunit 4 | 068878 | 119737 | 170634 | 24, 6, 4 |
| 2 | PRKRA | protein activator of interferon induced protein kinase EIF2AK2 | 180228 | 079156 | 204311 | 6, 38, 15 |
| 2 | PROC | protein C, inactivator of coagulation factors Va and VIIIa | 115718 | 169967 | 163166 | 19, 23, 17 |
| 2 | PDIA6 | protein disulfide isomerase family A member 6 | 143870 | 143882 | 162975 | 29, 13, 3 |
| 2 | PRKAG3 | protein kinase AMP-activated non-catalytic subunit gamma 3 | 115592 | 135929 | 115596 | 15, 25, 23 |
| 2 | PRKCE | protein kinase C epsilon | 171132 | 068784 | 116016 | 22, 8, 38 |
| 2 | PRKD3 | protein kinase D3 | 115825 | 003509 | 115828 | 22, 44, 21 |
| 2 | PKDCC | protein kinase domain containing, cytoplasmic | 162878 | 205086 | 143924 | 25, 12, 3 |
| 2 | PPP1CB | protein phosphatase 1 catalytic subunit beta | 213639 | 163803 | 163806 | 42, 45, 44 |
| 2 | PPP1R1C | protein phosphatase 1 regulatory inhibitor subunit 1C | 150722 | 138434 | 115252 | 40, 11, 10 |
| 2 | PPP1R21 | protein phosphatase 1 regulatory subunit 21 | 162869 | 170802 | 243244 | 13, 18, 1 |
| 2 | PPP1R7 | protein phosphatase 1 regulatory subunit 7 | 115685 | 115687 | 146205 | 41, 21, 40 |
| 2 | PPP3R1 | protein phosphatase 3 regulatory subunit B, alpha | 221823 | 115946 | 119865 | 37, 26, 5 |
| 2 | PPP4R3B | protein phosphatase 4 regulatory subunit 3B | 275052 | 163001 | 138035 | 42, 40, 22 |
| 2 | PPM1B | protein phosphatase, Mg2+/Mn2+ dependent 1B | 138032 | 138095 | 138079 | 11, 22, 36 |
| 2 | PPM1G | protein phosphatase, Mg2+/Mn2+ dependent 1G | 115241 | 163795 | 115216 | 13, 3, 43 |
| 2 | PTPN18 | protein tyrosine phosphatase, non-receptor type 18 | 072135 | 136718 | 196834 | 41, 36, 26 |
| 2 | PTPN4 | protein tyrosine phosphatase, non-receptor type 4 | 088179 | 144120 | 115109 | 17, 36, 44 |
| 2 | PTPRN | protein tyrosine phosphatase, receptor type N | 054356 | 135924 | 182698 | |
| 2 | PTMA | prothymosin, alpha | 187514 | 177673 | 156973 | 41, 30, 21 |
| 2 | PUS10 | pseudouridylate synthase 10 | 162927 | 162924 | 162928 | 22, 13, 42 |
| 2 | PUM2 | pumilio RNA binding family member 2 | 055917 | 115884 | 143878 | 22, 42, 23 |
| 2 | PDK1 | pyruvate dehydrogenase kinase 1 | 152256 | 091409 | 091428 | 1, 24, 42 |
| 2 | R3HDM1 | R3H domain containing 1 | 048991 | 121988 | 144224 | 45, 40, 8 |
| 2 | RABL2A | RAB, member of RAS oncogene family-like 2A | 144134 | 184492 | 115084 | 1, 40, 18 |
| 2 | RAB10 | RAB10, member RAS oncogene family | 084733 | 084731 | 157833 | 42, 22, 7 |
| 2 | RAB11FIP5 | RAB11 family interacting protein 5 | 135631 | 144040 | 214513 | 34, 38, 36 |
| 2 | RAB17 | RAB17, member RAS oncogene family | 124839 | 071677 | 124831 | |
| 2 | RAB1A | RAB1A, member RAS oncogene family | 138069 | 011523 | 138071 | 31, 29, 19 |
| 2 | RAB3GAP1 | RAB3 GTPase activating protein catalytic subunit 1 | 115839 | 176601 | 121988 | 22, 42, 24 |
| 2 | RAB6C | RAB6C, member RAS oncogene family | 222014 | 136720 | 196604 | 27, 23, 33 |
| 2 | WTH3DI | RAB6C-like | 233087 | 188219 | 173272 | 29, 24, 6 |
| 2 | RAD51AP2 | RAD51 associated protein 2 | 214842 | 197872 | 163032 | 38, |
| 2 | RSAD2 | radical S-adenosyl methionine domain containing 2 | 134321 | 134326 | 151692 | 42, 13, 5 |
| 2 | RFTN2 | raftlin family member 2 | 162944 | 115540 | 247626 | 11, 2, 8 |
| 2 | RANBP2 | RAN binding protein 2 | 153201 | 169756 | 163006 | 31, 22, 42 |
| 2 | RGPD1 | RANBP2-like and GRIP domain containing 1 | 187627 | 172116 | 183281 | 11, 39, 38 |
| 2 | RGPD2 | RANBP2-like and GRIP domain containing 2 | 185304 | 125551 | 172086 | 13, 32, 24 |
| 2 | RGPD3 | RANBP2-like and GRIP domain containing 3 | 153165 | 115652 | 144057 | 17, 30, 40 |
| 2 | RGPD4 | RANBP2-like and GRIP domain containing 4 | 196862 | 144057 | 115665 | 24, 44, 32 |
| 2 | RGPD5 | RANBP2-like and GRIP domain containing 5 | 015568 | 198142 | 256977 | |
| 2 | RGPD6 | RANBP2-like and GRIP domain containing 6 | 183054 | 256671 | 169679 | 11, 38, 40 |
| 2 | RGPD8 | RANBP2-like and GRIP domain containing 8 | 169629 | 188177 | 114999 | 26, 9, 30 |
| 2 | RAPGEF4 | Rap guanine nucleotide exchange factor 4 | 091428 | 152256 | 091436 | 8, 31, 37 |
| 2 | RAPH1 | Ras association (RalGDS/AF-6) and pleckstrin homology domains 1 | 173166 | 138443 | 178562 | 26, 11, 3 |
| 2 | RASGRP3 | RAS guanyl releasing protein 3 | 152689 | 049323 | 119812 | 22, 43, 42 |
| 2 | RHOB | ras homolog family member B | 143878 | 055917 | 118960 | 17, 45, 1 |
| 2 | RHOQ | ras homolog family member Q | 119729 | 250565 | 151665 | 31, 29, 26 |
| 2 | RALB | RAS like proto-oncogene B | 144118 | 226479 | 281676 | 29, 1, 43 |
| 2 | REEP1 | receptor accessory protein 1 | 068615 | 132313 | 115548 | 11, 8, 38 |
| 2 | RAMP1 | receptor activity modifying protein 1 | 132329 | 177483 | 184182 | 25, 33, 6 |
| 2 | RTP5 | receptor transporter protein 5 (putative) | 188011 | 188389 | 216921 | |
| 2 | REG1A | regenerating family member 1 alpha | 115386 | 172023 | 172016 | 28, 4, 37 |
| 2 | REG1B | regenerating family member 1 beta | 172023 | 143954 | 115386 | 28, 4, 37 |
| 2 | REG3A | regenerating family member 3 alpha | 172016 | 115386 | 066032 | 4, 37, 16 |
| 2 | REG3G | regenerating family member 3 gamma | 143954 | 176204 | 172023 | |
| 2 | RESP18 | regulated endocrine specific protein 18 | 182698 | 054356 | 123992 | |
| 2 | RMDN2 | regulator of microtubule dynamics 2 | 115841 | 163171 | 138061 | 10, 38, 2 |
| 2 | REL | REL proto-oncogene, NF-kB subunit | 162924 | 115421 | 162927 | 22, 5, 21 |
| 2 | RIF1 | replication timing regulatory factor 1 | 080345 | 123610 | 183091 | |
| 2 | RPRM | reprimo, TP53 dependent G2 arrest mediator candidate | 177519 | 177917 | 144278 | 31, 20, 8 |
| 2 | RMND5A | required for meiotic nuclear division 5 homolog A | 153561 | 239305 | 153563 | 30, 29, 39 |
| 2 | RTN4 | reticulon 4 | 115310 | 214595 | 162994 | |
| 2 | RDH14 | retinol dehydrogenase 14 (all-trans/9-cis/11-cis) | 240857 | 170745 | 250741 | 41, 31, 33 |
| 2 | RETSAT | retinol saturase | 042445 | 152291 | 115459 | 24, 1, 2 |
| 2 | REV1 | REV1, DNA directed polymerase | 135945 | 158417 | 144218 | 1, 26, 22 |
| 2 | RFX8 | RFX family member 8, lacking RFX DNA binding domain | 196460 | 175874 | 071054 | 40, 45, 16 |
| 2 | ROCK2 | Rho associated coiled-coil containing protein kinase 2 | 134318 | 162976 | 169016 | 22, 26, 45 |
| 2 | RND3 | Rho family GTPase 3 | 115963 | 168288 | 184898 | 1, 45, 35 |
| 2 | ARHGAP15 | Rho GTPase activating protein 15 | 075884 | 115919 | 121964 | |
| 2 | ARHGAP25 | Rho GTPase activating protein 25 | 163219 | 169618 | 163217 | |
| 2 | ARHGEF33 | Rho guanine nucleotide exchange factor 33 | 214694 | 188010 | 115904 | 28, 27, 20 |
| 2 | ARHGEF4 | Rho guanine nucleotide exchange factor 4 | 136002 | 178171 | 152102 | 4, 8, 14 |
| 2 | RHBDD1 | rhomboid domain containing 1 | 144468 | 169047 | 081052 | 22, 39, 24 |
| 2 | RTKN | rhotekin | 114993 | 005448 | 115274 | 4, 35, 16 |
| 2 | RBKS | ribokinase | 171174 | 243147 | 158019 | 10, 29, 17 |
| 2 | RNASEH1 | ribonuclease H1 | 171865 | 255767 | 171863 | 43, 11, 45 |
| 2 | RRM2 | ribonucleotide reductase regulatory subunit M2 | 171848 | 205795 | 115756 | 13, 39, 3 |
| 2 | RPIA | ribose 5-phosphate isomerase A | 153574 | 172071 | 211592 | 9, 11, 22 |
| 2 | RPL31 | ribosomal protein L31 | 071082 | 170485 | 204634 | 43, 7, 25 |
| 2 | RPL37A | ribosomal protein L37a | 197756 | 138375 | 115457 | 25, 33, 43 |
| 2 | RPS27A | ribosomal protein S27a | 143947 | 162994 | 085760 | |
| 2 | RPS7 | ribosomal protein S7 | 171863 | 171865 | 118004 | |
| 2 | RPE | ribulose-5-phosphate-3-epimerase | 197713 | 144406 | 144445 | 4, 45, 34 |
| 2 | RNF103 | ring finger protein 103 | 239305 | 249884 | 153561 | 11, 31, 2 |
| 2 | RNF144A | ring finger protein 144A | 151692 | 134321 | 115738 | 11, 29, 31 |
| 2 | RNF149 | ring finger protein 149 | 163162 | 158435 | 175874 | 1, 11, 5 |
| 2 | RNF181 | ring finger protein 181 | 168894 | 168899 | 168890 | 31, 43, 19 |
| 2 | RNF25 | ring finger protein 25 | 163481 | 074582 | 163482 | 31, 23, 1 |
| 2 | RBM43 | RNA binding motif protein 43 | 184898 | 115963 | 123609 | 29, 20, 24 |
| 2 | RBM44 | RNA binding motif protein 44 | 177483 | 124831 | 132329 | 10, 38, 8 |
| 2 | RBM45 | RNA binding motif protein 45 | 155636 | 128655 | 079156 | 13, 15, 4 |
| 2 | RBMS1 | RNA binding motif single stranded interacting protein 1 | 153250 | 115221 | 136560 | |
| 2 | POLR1A | RNA polymerase I subunit A | 068654 | 115525 | 132300 | 22, 21, 15 |
| 2 | POLR1B | RNA polymerase I subunit B | 125630 | 114999 | 125611 | 38, 25, 42 |
| 2 | POLR2D | RNA polymerase II subunit D | 144231 | 173349 | 144233 | 13, 29, 44 |
| 2 | RNF103- | RNF103-CHMP3 readthrough | 249884 | 115561 | 239305 | |
| | CHMP3 | | | | | |
| 2 | RUFY4 | RUN and FYVE domain containing 4 | 188282 | 079308 | 180871 | 39, 27, 36 |
| 2 | SRBD1 | S1 RNA binding domain 1 | 068784 | 170577 | 171132 | 42, 36, 25 |
| 2 | SAG | S-antigen visual arrestin | 130561 | 085978 | 077044 | 14, 25, 10 |
| 2 | SATB2 | SATB homeobox 2 | 119042 | 115896 | 226124 | 19, 8, 18 |
| 2 | SESTD1 | SEC14 and spectrin domain containing 1 | 187231 | 163492 | 144331 | 26, 29, 11 |
| 2 | SCRN3 | secern in 3 | 144306 | 138433 | 163328 | 4, 34, 43 |
| 2 | SPP2 | secreted phosphoprotein 2 | 072080 | 144481 | 188042 | 19, 25, |
| 2 | SCTR | secretin receptor | 080293 | 171227 | 163075 | 20, 28, 25 |
| 2 | SCG2 | secretogranin II | 171951 | 152049 | 152056 | 8, 28, 31 |
| 2 | SCLY | selenocysteine lyase | 132330 | 258984 | 144488 | 18, 9, 3 |
| 2 | SELENOI | selenoprotein I | 138018 | 173567 | 157856 | 29, 22, 26 |
| 2 | SEMA4C | semaphorin 4C | 168758 | 213337 | 168754 | 25, 12, 26 |
| 2 | SPR | sepiapterin reductase (7,8-dihydrobiopterin: NADP+ oxidoreductase) | 116096 | 144036 | 135638 | 29, 35, 34 |
| 2 | SEPT10 | septin 10 | 186522 | 279515 | 198142 | 13, 45, 38 |
| 2 | SEPT2 | septin 2 | 168385 | 115677 | 006607 | 42, 11, 24 |
| 2 | SRSF7 | serine and arginine rich splicing factor 7 | 115875 | 143891 | 152147 | 4, 34, 1 |
| 2 | STK11IP | serine/threonine kinase 11 interacting protein | 144589 | 123999 | 114923 | 13, 1, 41 |
| 2 | STK16 | serine/threonine kinase 16 | 115661 | 163521 | 127824 | 21, 5, 13 |
| 2 | STK17B | serine/threonine kinase 17b | 081320 | 118997 | 138411 | 22, 41, 13 |
| 2 | STK25 | serine/threonine kinase 25 | 115694 | 006607 | 176720 | 41, 34, 22 |
| 2 | STK36 | serine/threonine kinase 36 | 163482 | 163481 | 135912 | 43, 26, 38 |
| 2 | STK39 | serine/threonine kinase 39 | 198648 | 172318 | 172292 | 22, 4, 33 |
| 2 | SERPINE2 | serpin family E member 2 | 135919 | 135900 | 124019 | 10, 45, 32 |
| 2 | SERTAD2 | SERTA domain containing 2 | 179833 | 279644 | 115902 | 39, 15, 13 |
| 2 | SDPR | serum deprivation response | 168497 | 173559 | 144339 | 1, 43, 45 |
| 2 | SMYD1 | SET and MYND domain containing 1 | 115593 | 172086 | 163586 | 15, 25, 18 |
| 2 | SFT2D3 | SFT2 domain containing 3 | 173349 | 136709 | 144231 | 22, 28, 16 |
| 2 | SH2D6 | SH2 domain containing 6 | 152292 | 042493 | 168906 | |
| 2 | SH3YL1 | SH3 and SYLF domain containing 1 | 035115 | 184731 | 143727 | |
| 2 | SH3BP4 | SH3 domain binding protein 4 | 130147 | 188042 | 157985 | 43, 42, 23 |
| 2 | SH3RF3 | SH3 domain containing ring finger 3 | 172985 | 135960 | 279515 | 10, 16, 15 |
| 2 | SGO2 | shugoshin 2 | 163535 | 155729 | 138356 | 22, 34, 39 |
| 2 | SFXN5 | sideroflexin 5 | 144040 | 135638 | 135631 | 8, 34, 26 |
| 2 | STAT1 | signal transducer and activator of transcription 1 | 115415 | 115419 | 138378 | 42, 44, 43 |
| 2 | STAT4 | signal transducer and activator of transcription 4 | 138378 | 115415 | 128641 | 36, 18, 31 |
| 2 | STAM2 | signal transducing adaptor molecule 2 | 115145 | 182389 | 157827 | 25, 33, 45 |
| 2 | SAP130 | Sin3A associated protein 130 | 136715 | 144233 | 136731 | 38, 18, 24 |
| 2 | SIX2 | SIX homeobox 2 | 170577 | 138083 | 068784 | 9, 20, 25 |
| 2 | SIX3 | SIX homeobox 3 | 138083 | 143919 | 170577 | 14, 20, 17 |
| 2 | SSB | Sjogren syndrome antigen B | 138385 | 213160 | 138382 | 7, 4, 34 |
| 2 | SNRPG | small nuclear ribonucleoprotein polypeptide G | 143977 | 116005 | 035141 | |
| 2 | SNRNP27 | small nuclear ribonucleoprotein U4/U6.U5 subunit 27 | 124380 | 087338 | 059728 | 34, 7, 4 |
| 2 | SNRNP200 | small nuclear ribonucleoprotein U5 subunit 200 | 144028 | 144021 | 198885 | 18, 43, 22 |
| 2 | SUM01 | small ubiquitin-like modifier 1 | 116030 | 283310 | 055044 | 29, 34, 7 |
| 2 | SMYD5 | SMYD family member 5 | 135632 | 214513 | 135617 | 33, 4, 13 |
| 2 | SCN1A | sodium voltage-gated channel alpha subunit 1 | 144285 | 123607 | 169432 | 8, 25, 14 |
| 2 | SCN2A | sodium voltage-gated channel alpha subunit 2 | 136531 | 153253 | 178662 | 8, 25, 20 |
| 2 | SCN3A | sodium voltage-gated channel alpha subunit 3 | 153253 | 169507 | 136531 | 26, 42, 24 |
| 2 | SCN7A | sodium voltage-gated channel alpha subunit 7 | 136546 | 169432 | 163092 | 11, 26, 25 |
| 2 | SCN9A | sodium voltage-gated channel alpha subunit 9 | 169432 | 144285 | 136546 | 26, 24, 32 |
| 2 | SLC1A4 | solute carrier family 1 member 4 | 115902 | 179833 | 011523 | 21, 2, 42 |
| 2 | SLC11A1 | solute carrier family 11 member 1 | 018280 | 158428 | 144579 | 5, 36, 20 |
| 2 | SLC16A14 | solute carrier family 16 member 14 | 163053 | 153832 | 135899 | 42, 13, 24 |
| 2 | SLC19A3 | solute carrier family 19 member 3 | 135917 | 042304 | 115009 | 1, 32, 26 |
| 2 | SLC20A1 | solute carrier family 20 member 1 | 144136 | 125611 | 144130 | 34, 44, 7 |
| 2 | SLC23A3 | solute carrier family 23 member 3 | 213901 | 280537 | 115649 | 17, 9, 4 |
| 2 | SLC25A12 | solute carrier family 25 member 12 | 115840 | 077380 | 128708 | 26, 43, 24 |
| 2 | SLC3A1 | solute carrier family 3 member 1 | 138079 | 138032 | 138078 | 17, 4, 16 |
| 2 | SLC30A3 | solute carrier family 30 member 3 | 115194 | 084774 | 163793 | 38, 8, 6 |
| 2 | SLC30A6 | solute carrier family 30 member 6 | 152683 | 021574 | 091106 | 7, 42, 24 |
| 2 | SLC35F5 | solute carrier family 35 member F5 | 115084 | 144134 | 115091 | 13, 22, 2 |
| 2 | SLC35F6 | solute carrier family 35 member F6 | 213699 | 171303 | 115163 | 43, 29, 23 |
| 2 | SLC38A11 | solute carrier family 38 member 11 | 169507 | 082438 | 153253 | 36, 14, 30 |
| 2 | SLC39A10 | solute carrier family 39 member 10 | 196950 | 144339 | 118997 | 12, 33, 24 |
| 2 | SLC4A1AP | solute carrier family 4 member 1 adaptor protein | 163798 | 119760 | 243147 | 10, 13, 7 |
| 2 | SLC4A10 | solute carrier family 4 member 10 | 144290 | 136535 | 197635 | 8, 28, 22 |
| 2 | SLC4A3 | solute carrier family 4 member 3 | 114923 | 144589 | 116106 | 26, 15, 34 |
| 2 | SLC4A5 | solute carrier family 4 member 5 | 188687 | 264324 | 204843 | 40, 29, 24 |
| 2 | SLC40A1 | solute carrier family 40 member 1 | 138449 | 115368 | 138381 | 29, 25, 44 |
| 2 | SLC5A6 | solute carrier family 5 member 6 | 138074 | 163792 | 138085 | 37, 32, 41 |
| 2 | SLC5A7 | solute carrier family 5 member 7 | 115665 | 196862 | 196228 | 26, 2, 8 |
| 2 | SLC8A1 | solute carrier family 8 member A1 | 183023 | 138050 | 205086 | |
| 2 | SLC9A2 | solute carrier family 9 member A2 | 115616 | 180251 | 135953 | 29, 33, 3 |
| 2 | SLC9A4 | solute carrier family 9 member A4 | 180251 | 115607 | 115616 | 30, 24, 17 |
| 2 | SNX17 | sorting nexin 17 | 115234 | 115211 | 163795 | 13, 43, 36 |
| 2 | SOS1 | SOS Ras/Rac guanine nucleotide exchange factor 1 | 115904 | 214694 | 205111 | 22, 9, 40 |
| 2 | SOWAHC | sosondowah ankyrin repeat domain family member C | 198142 | 186522 | 015568 | 4, 9, 26 |
| 2 | SP100 | SP100 nuclear antigen | 067066 | 185404 | 135932 | 4, 30, 37 |
| 2 | SP110 | SP110 nuclear body protein | 135899 | 163053 | 280755 | 7, 41, 21 |
| 2 | SP140 | SP140 nuclear body protein | 079263 | 280755 | 185404 | 41, 22, 30 |
| 2 | SP140L | SP140 nuclear body protein like | 185404 | 079263 | 067066 | 33, 18, 39 |
| 2 | SP3 | Sp3 transcription factor | 172845 | 144354 | 138430 | 24, 18, 22 |
| 2 | SP5 | Sp5 transcription factor | 204335 | 071909 | 204334 | 19, 21, 12 |
| 2 | SP9 | Sp9 transcription factor | 217236 | 138430 | 138433 | 38, |
| 2 | SPAST | spastin | 021574 | 162961 | 152683 | 40, 13, 11 |
| 2 | SPC25 | SPC25, NDC80 kinetochore complex component | 152253 | 163072 | 152254 | 2, 22, 21 |
| 2 | SPOPL | speckle type BTB/POZ protein like | 144228 | 150540 | 144227 | 22, 1, 23 |
| 2 | SPTBN1 | spectrin beta, non-erythrocytic 1 | 115306 | 177994 | 214595 | 1, 29, 24 |
| 2 | SPDYA | speedy/RINGO cell cycle regulator family member A | 163806 | 213639 | 171103 | 10, 38, 26 |
| 2 | SPEG | SPEG complex locus | 072195 | 175084 | 144591 | 26, 31, 44 |
| 2 | SPAG16 | sperm associated antigen 16 | 144451 | 030419 | 174453 | 28, 38, 26 |
| 2 | SSFA2 | sperm specific antigen 2 | 138434 | 162992 | 150722 | 28, 43, 18 |
| 2 | SPATA3 | spermatogenesis associated 3 | 173699 | 135898 | 204128 | 38, 27, 8 |
| 2 | SPATS2L | spermatogenesis associated serine rich 2 like | 196141 | 162972 | 155729 | |
| 2 | SMPD4 | sphingomyelin phosphodiesterase 4 | 136699 | 152076 | 152082 | 42, 38, 39 |
| 2 | SGPP2 | sphingosine-1-phosphate phosphatase 2 | 163082 | 163081 | 116120 | 26, 37, 28 |
| 2 | SPHKAP | SPHK1 interactor, AKAP domain containing | 153820 | 123977 | 153823 | 15, 8, 20 |
| 2 | SF3B1 | splicing factor 3b subunit 1 | 115524 | 283224 | 115520 | 40, 24, 11 |
| 2 | SF3B6 | splicing factor 3b subunit 6 | 115128 | 119782 | 219626 | |
| 2 | SPRED2 | sprouty related EVH1 domain containing 2 | 198369 | 138071 | 143995 | 22, 29, 37 |
| 2 | SUPT7L | SPT7-like STAGA complex gamma subunit | 119760 | 198522 | 163798 | 39, 40, 36 |
| 2 | SOX11 | SRY-box 11 | 176887 | 214866 | 134326 | 12, 45, 14 |
| 2 | SEMA4F | ssemaphorin 4F | 135622 | 159374 | 159399 | 42, 8, 6 |
| 2 | ST3GAL5 | ST3 beta-galactoside alpha-2,3-sialyltransferase 5 | 115525 | 168874 | 068654 | 5, |
| 2 | ST6GAL2 | ST6 beta-galactoside alpha-2,6-sialyltransferase 2 | 144057 | 153165 | 196862 | 40, 12, 38 |
| 2 | STAMBP | STAM binding protein | 124356 | 187833 | 163017 | |
| 2 | STARD7 | StAR related lipid transfer domain containing 7 | 084090 | 158050 | 135956 | 34, 21, 4 |
| 2 | STRADB | STE20-related kinase adaptor beta | 082146 | 115993 | 155754 | 1, 17, 29 |
| 2 | STEAP3 | STEAP3 metalloreductase | 115107 | 144119 | 186132 | 29, 26, 7 |
| 2 | SRD5A2 | steroid 5 alpha-reductase 2 | 277893 | 158125 | 162959 | 42, 33, 14 |
| 2 | STON1- | STON1-GTF2A1L readthrough | 068781 | 243244 | 242441 | |
| | GTF2A1L | | | | | |
| 2 | STON1 | stonin 1 | 243244 | 162869 | 068781 | |
| 2 | STRN | striatin | 115808 | 205221 | 008869 | 29, 15, 22 |
| 2 | SMC6 | structural maintenance of chromosomes 6 | 163029 | 163032 | 178295 | |
| 2 | SUCLG1 | succinate-CoA ligase alpha subunit | 163541 | 162951 | 115423 | 17, 11, 25 |
| 2 | SULT1C2 | sulfotransferase family 1C member 2 | 198203 | 196228 | 198075 | 37, 17, 29 |
| 2 | SULT1C3 | sulfotransferase family 1C member 3 | 196228 | 115665 | 198203 | 14, |
| 2 | SULT1C4 | sulfotransferase family 1C member 4 | 198075 | 198203 | 135968 | 1, 15, 10 |
| 2 | SULT6B1 | sulfotransferase family 6B member 1 | 138068 | 055332 | 218739 | |
| 2 | SOCS5 | suppressor of cytokine signaling 5 | 171150 | 119878 | 180398 | 1, 42, 6 |
| 2 | SFTPB | surfactant protein B | 168878 | 168887 | 115523 | 20, 40, 10 |
| 2 | SNED1 | sushi, nidogen and EGF like domains 1 | 162804 | 226321 | 122085 | 34, 26, 40 |
| 2 | SMARCAL1 | SWI/SNF related, matrix associated, actin dependent regulator of chromatin, | 138375 | 144583 | 197756 | 7, 43, 24 |
| | | subfamily a like 1 | | | | |
| 2 | SDC1 | syndecan 1 | 115884 | 068697 | 055917 | 13, 32, 37 |
| 2 | SNTG2 | syntrophin gamma 2 | 172554 | 280360 | 115705 | 10, 38, 42 |
| 2 | TACR1 | tachykinin receptor 1 | 115353 | 115350 | 115363 | 1, 42, 44 |
| 2 | TAF1B | TATA-box binding protein associated factor, RNA polymerase I subunit B | 115750 | 134308 | 134317 | 19, 13, 18 |
| 2 | TBC1D8 | TBC1 domain family member 8 | 204634 | 071082 | 158435 | 37, 13, 34 |
| 2 | TBR1 | T-box, brain 1 | 136535 | 115233 | 144290 | 8, |
| 2 | TLX2 | T-cell leukemia homeobox 2 | 115297 | 115289 | 144045 | 14, 22, 33 |
| 2 | TEKT4 | tektin 4 | 163060 | 259916 | 172005 | 42, 20, 38 |
| 2 | TNS1 | tensin 1 | 079308 | 231672 | 188282 | 1, 40, 6 |
| 2 | TEX261 | testis expressed 261 | 144043 | 144031 | 124357 | 27, 26, 22 |
| 2 | TEX37 | testis expressed 37 | 172073 | 214336 | 172071 | 38, 32, |
| 2 | TSGA10 | testis specific 10 | 135951 | 196872 | 273045 | |
| 2 | TET3 | tet methylcytosine dioxygenase 3 | 187605 | 114956 | 163170 | |
| 2 | TTC21B | tetratricopeptide repeat domain 21B | 123607 | 115339 | 144285 | 4, 31, 15 |
| 2 | TTC27 | tetratricopeptide repeat domain 27 | 018699 | 115760 | 049323 | |
| 2 | TTC30A | tetratricopeptide repeat domain 30A | 197557 | 196659 | 128655 | 7, 38, 11 |
| 2 | TTC30B | tetratricopeptide repeat domain 30B | 196659 | 018510 | 197557 | 42, 2, 7 |
| 2 | TTC31 | tetratricopeptide repeat domain 31 | 115282 | 135637 | 179528 | 9, 22, 38 |
| 2 | TTC32 | tetratricopeptide repeat domain 32 | 183891 | 143867 | 118965 | 29, 34, 17 |
| 2 | TTC7A | tetratricopeptide repeat domain 7A | 068724 | 180398 | 239605 | 22, 7, 1 |
| 2 | TANC1 | tetratricopeptide repeat, ankyrin repeat and coiled-coil containing 1 | 115183 | 163331 | 196151 | 11, 31, 3 |
| 2 | THADA | THADA, armadillo repeat containing | 115970 | 152518 | 152527 | 30, 10, 40 |
| 2 | THAP4 | THAP domain containing 4 | 176946 | 176720 | 168397 | 29, 9, 18 |
| 2 | TXNDC9 | thioredoxin domain containing 9 | 115514 | 144214 | 158417 | 4, 7, 34 |
| 2 | THNSL2 | threonine synthase like 2 | 144115 | 163586 | 214336 | 27, 24, 33 |
| 2 | THSD7B | thrombospondin type 1 domain containing 7B | 144229 | 121966 | 150540 | 45, 25, 37 |
| 2 | THUMPD2 | THUMP domain containing 2 | 138050 | 152154 | 183023 | 29, 9, 25 |
| 2 | TMSB10 | thymosin beta 10 | 034510 | 279992 | 176407 | 43, 7, 45 |
| 2 | TRIP12 | thyroid hormone receptor interactor 12 | 153827 | 187957 | 153832 | 22, 4, 6 |
| 2 | TPO | thyroid peroxidase | 115705 | 172554 | 130508 | 40, 1, 18 |
| 2 | TIA1 | TIA1 cytotoxic granule associated RNA binding protein | 116001 | 115998 | 116005 | 22, 11, 1 |
| 2 | TIGD1 | tigger transposable element derived 1 | 221944 | 196811 | 135930 | 28, 25, 36 |
| 2 | TFPI | tissue factor pathway inhibitor | 003436 | 064989 | 144366 | 32, 43, 6 |
| 2 | TTN | titin | 155657 | 116095 | 283186 | 25, 18, 15 |
| 2 | TNFAIP6 | TNF alpha induced protein 6 | 123610 | 123609 | 080345 | 11, 6, 45 |
| 2 | TLK1 | tousled like kinase 1 | 198586 | 115806 | 123600 | 40, 26, 13 |
| 2 | TPRKB | TP53RK binding protein | 144034 | 204872 | 144048 | 11, 17, 7 |
| 2 | TRABD2A | TraB domain containing 2A | 186854 | 115423 | 279992 | 16, 39, 10 |
| 2 | TANK | TRAF family member associated NFKB activator | 136560 | 153250 | 115233 | 5, 31, 4 |
| 2 | TRAF3IP1 | TRAF3 interacting protein 1 | 204104 | 132326 | 065802 | 11, 26, 14 |
| 2 | TRAK2 | trafficking kinesin protein 2 | 115993 | 155749 | 082146 | 29, 40, 35 |
| 2 | TRAPPC12 | trafficking protein particle complex 12 | 171853 | 032389 | 182551 | 7, 22, 18 |
| 2 | TCF23 | transcription factor 23 | 163792 | 186143 | 138074 | 44, |
| 2 | TCF7L1 | transcription factor 7 like 1 | 152284 | 176407 | 152291 | 23, 12, 1 |
| 2 | TFCP2L1 | transcription factor CP2 like 1 | 115112 | 074047 | 074054 | 17, 40, 23 |
| 2 | TGFA | transforming growth factor alpha | 163235 | 035141 | 075340 | 40, 42, 24 |
| 2 | TGFBRAP1 | transforming growth factor beta receptor associated protein 1 | 135966 | 135973 | 135974 | 23, 18, 9 |
| 2 | TGOLN2 | trans-golgi network protein 2 | 152291 | 152284 | 042445 | 6, 40, 29 |
| 2 | TRPM8 | transient receptor potential cation channel subfamily M member 8 | 144481 | 123485 | 072080 | 33, 19, 45 |
| 2 | TNP1 | transition protein 1 | 118245 | 115461 | 278951 | 38, 17, 8 |
| 2 | TSN | translin | 211460 | 155438 | 155052 | 4, 13, 45 |
| 2 | TM4SF20 | transmembrane 4 L six family member 20 | 168955 | 168958 | 173744 | 16, 37, 25 |
| 2 | TMBIM1 | transmembrane BAX inhibitor motif containing 1 | 135926 | 127838 | 158428 | 18, 24, 4 |
| 2 | TMEM127 | transmembrane protein 127 | 135956 | 084090 | 144021 | 43, 26, 27 |
| 2 | TMEM131 | transmembrane protein 131 | 075568 | 115085 | 168658 | 22, 11, 42 |
| 2 | TMEM150A | transmembrane protein 150A | 168890 | 168894 | 168883 | 32, 26, 31 |
| 2 | TMEM163 | transmembrane protein 163 | 152128 | 152127 | 153086 | 30, 38, 26 |
| 2 | TMEM169 | transmembrane protein 169 | 163449 | 115425 | 079246 | 39, 28, 8 |
| 2 | TMEM17 | transmembrane protein 17 | 186889 | 170340 | 115504 | 13, 23, 28 |
| 2 | TMEM177 | transmembrane protein 177 | 144120 | 163075 | 088179 | 21, 34, 35 |
| 2 | TMEM178A | transmembrane protein 178A | 152154 | 011566 | 138050 | 14, 18, 17 |
| 2 | TMEM18 | transmembrane protein 18 | 151353 | 189292 | 280360 | 29, 15, 22 |
| 2 | TMEM182 | transmembrane protein 182 | 170417 | 135953 | 198914 | 15, 31, 7 |
| 2 | TMEM185B | transmembrane protein 185B | 226479 | 115109 | 144118 | 24, 4, 29 |
| 2 | TMEM198 | transmembrane protein 198 | 188760 | 123989 | 124006 | 38, 3, 27 |
| 2 | TMEM214 | transmembrane protein 214 | 119777 | 084764 | 084693 | 33, 40, 18 |
| 2 | TMEM237 | transmembrane protein 237 | 155755 | 155754 | 082126 | 18, 10, 6 |
| 2 | TMEM247 | transmembrane protein 247 | 187600 | 116016 | 250565 | 38, 32, |
| 2 | TMEM37 | transmembrane protein 37 | 171227 | 155368 | 080293 | 29, 17, 28 |
| 2 | TMEM87B | transmembrane protein 87B | 153214 | 153208 | 144152 | 24, 22, 29 |
| 2 | TMEFF2 | transmembrane protein with EGF like and two follistatin like domains 2 | 144339 | 168497 | 196950 | 26, 33, 8 |
| 2 | TRIB2 | tribbles pseudokinase 2 | 071575 | 134324 | 162981 | 42, 26, 22 |
| 2 | TRIM43 | tripartite motif containing 43 | 144015 | 115042 | 174501 | |
| 2 | TRIM54 | tripartite motif containing 54 | 138100 | 163793 | 163794 | 40, 25, 41 |
| 2 | TRMT61B | tRNA methyltransferase 61B | 171103 | 163806 | 163811 | 11, 13, 17 |
| 2 | TYW5 | tRNA-yW synthesizing protein 5 | 162971 | 178074 | 162972 | 36, 11, 42 |
| 2 | TSPYL6 | TSPY like 6 | 178021 | 170634 | 177994 | 38, 27, |
| 2 | TUBA3D | tubulin alpha 3d | 075886 | 173272 | 163040 | 38, 15, 23 |
| 2 | TUBA3E | tubulin alpha 3e | 152086 | 152082 | 136710 | 38, |
| 2 | TUBA4A | tubulin alpha 4a | 127824 | 115661 | 243910 | 21, 25, 41 |
| 2 | TUBA4B | Tubulin alpha 4b | 243910 | 127824 | 135924 | 34, 30, 21 |
| 2 | TTL | tubulin tyrosine ligase | 114999 | 169629 | 125630 | 29, 11, 9 |
| 2 | TTLL4 | tubulin tyrosine ligase like 4 | 135912 | 163482 | 135929 | 38, 41, 22 |
| 2 | TDRD15 | tudor domain containing 15 | 218819 | 084674 | 119771 | |
| 2 | TP53I3 | tumor protein p53 inducible protein 3 | 115129 | 219626 | 176732 | 6, 25, 26 |
| 2 | TSSC1 | tumor suppressing subtransferable candidate 1 | 032389 | 186487 | 171853 | 9, 29, 35 |
| 2 | TWIST2 | twist family bHLH transcription factor 2 | 233608 | 065802 | 068024 | 10, 15, 6 |
| 2 | YWHAQ | tyrosine 3-monooxygenase/tryptophan 5-monooxygenase activation protein | 134308 | 151694 | 115750 | 43, 22, 5 |
| | | theta | | | | |
| 2 | UBE2F-SCLY | UBE2F-SCLY readthrough (NMD candidate) | 258984 | 184182 | 132330 | 15, 32, 5 |
| 2 | UBE2E3 | ubiquitin conjugating enzyme E2 E3 | 170035 | 163510 | 115232 | |
| 2 | UBE2F | ubiquitin conjugating enzyme E2 F (putative) | 184182 | 132329 | 258984 | 15, 32, 5 |
| 2 | UBR3 | ubiquitin protein ligase E3 component n-recognin 3 (putative) | 144357 | 138382 | 071909 | 42, 29, 26 |
| 2 | USP34 | ubiquitin specific peptidase 34 | 115464 | 173209 | 082898 | 23, 26, 22 |
| 2 | USP37 | ubiquitin specific peptidase 37 | 135913 | 127831 | 144580 | 26, 42, 38 |
| 2 | USP39 | ubiquitin specific peptidase 39 | 168883 | 168890 | 168887 | 43, 39, 5 |
| 2 | USP40 | ubiquitin specific peptidase 40 | 085982 | 077044 | 242366 | 24, 13, 42 |
| 2 | UBXN2A | UBX domain protein 2A | 173960 | 119778 | 205639 | 40, 1, 4 |
| 2 | UBXN4 | UBX domain protein 4 | 144224 | 048991 | 115850 | 22, 4, 13 |
| 2 | UGT1A1 | UDP glucuronosyltransferase family 1 member A1 | 241635 | 243135 | 185038 | 13, 17, 24 |
| 2 | UGT1A10 | UDP glucuronosyltransferase family 1 member A10 | 242515 | 242366 | 241119 | |
| 2 | UGT1A3 | UDP glucuronosyltransferase family 1 member A3 | 243135 | 244474 | 241635 | |
| 2 | UGT1A4 | UDP glucuronosyltransferase family 1 member A4 | 244474 | 240224 | 243135 | |
| 2 | UGT1A5 | UDP glucuronosyltransferase family 1 member A5 | 240224 | 167165 | 244474 | |
| 2 | UGT1A6 | UDP glucuronosyltransferase family 1 member A6 | 167165 | 244122 | 240224 | 13, 38, |
| 2 | UGT1A7 | UDP glucuronosyltransferase family 1 member A7 | 244122 | 241119 | 167165 | |
| 2 | UGT1A8 | UDP glucuronosyltransferase family 1 member A8 | 242366 | 085982 | 242515 | |
| 2 | UGT1A9 | UDP glucuronosyltransferase family 1 member A9 | 241119 | 242515 | 244122 | |
| 2 | B3GNT2 | UDP-GlcNAc: betaGal beta-1,3-N-acetylglucosaminyltransferase 2 | 170340 | 280544 | 186889 | 4, 2, 12 |
| 2 | B3GNT7 | UDP-GlcNAc: betaGal beta-1,3-N-acetylglucosaminyltransferase 7 | 156966 | 135931 | 115053 | 13, 20, 10 |
| 2 | UGGT1 | UDP-glucose glycoprotein glucosyltransferase 1 | 136731 | 136715 | 136720 | 24, 22, 26 |
| 2 | UGP2 | UDP-glucose pyrophosphorylase 2 | 169764 | 281071 | 143952 | 15, 39, 13 |
| 2 | UXS1 | UDP-glucuronate decarboxylase 1 | 115652 | 119147 | 153165 | 23, 16, |
| 2 | UNC50 | unc-50 inner nuclear membrane RNA binding protein | 115446 | 183513 | 071073 | 45, 31, 28 |
| 2 | UNC80 | unc-80 homolog, NALCN activator | 144406 | 078018 | 197713 | 31, 8, 29 |
| 2 | TISP43 | Uncharacterized LOC150527 | 183292 | 184761 | 136698 | |
| 2 | UPP2 | uridine phosphorylase 2 | 007001 | 115170 | 153237 | 17, 19, 8 |
| 2 | UCN | urocortin | 163794 | 138100 | 115204 | 6, 2, 27 |
| 2 | VRK2 | vaccinia related kinase 2 | 028116 | 055813 | 115392 | |
| 2 | VAX2 | ventral anterior homeobox 2 | 116035 | 116031 | 116039 | 41, 14, 27 |
| 2 | VAMP5 | vesicle associated membrane protein 5 | 168899 | 281331 | 168894 | 25, 15, 34 |
| 2 | VAMP8 | vesicle associated membrane protein 8 | 118640 | 115486 | 281331 | 31, 5, 30 |
| 2 | VIL1 | villin 1 | 127831 | 144579 | 135913 | 16, 19, 3 |
| 2 | VSNL1 | visinin like 1 | 163032 | 214842 | 163029 | 8, 25, 16 |
| 2 | VIT | vitrin | 205221 | 171055 | 115808 | 26, 27, 42 |
| 2 | MYCN | v-myc avian myelocytomatosis viral oncogene neuroblastoma derived homolog | 134323 | 079785 | 197872 | 14, 12, 8 |
| 2 | VWA3B | von Willebrand factor A domain containing 3B | 168658 | 075568 | 144191 | 38, 42, 39 |
| 2 | VWC2L | von Willebrand factor C domain containing protein 2-like | 174453 | 144451 | 138376 | 8, |
| 2 | VPS54 | VPS54, GARP complex subunit | 143952 | 169764 | 197329 | 2, 13, 24 |
| 2 | WIPF1 | WAS/WASL interacting protein family member 1 | 115935 | 163328 | 138435 | |
| 2 | WDCP | WD repeat and coiled coil containing | 163026 | 205639 | 119782 | 39, 25, 32 |
| 2 | WDFY1 | WD repeat and FYVE domain containing 1 | 085449 | 152056 | 135900 | 31, 35, 45 |
| 2 | WDPCP | WD repeat containing planar cell polarity effector | 143951 | 115507 | 014641 | |
| 2 | WDR12 | WD repeat domain 12 | 138442 | 163596 | 138380 | 6, 13, 40 |
| 2 | WDR33 | WD repeat domain 33 | 136709 | 144230 | 173349 | 1, 22, 27 |
| 2 | WDR35 | WD repeat domain 35 | 118965 | 183891 | 132031 | 29, 40, 1 |
| 2 | WDR43 | WD repeat domain 43 | 163811 | 171103 | 189350 | 3, 13, 42 |
| 2 | WDR54 | WD repeat domain 54 | 005448 | 159239 | 114993 | 30, 43, 39 |
| 2 | WDR75 | WD repeat domain 75 | 115368 | 204262 | 138449 | 18, 42, 45 |
| 2 | WDR92 | WD repeat domain 92 | 243667 | 197223 | 273398 | 30, 2, 45 |
| 2 | WDSUB1 | WD repeat, sterile alpha motif and U-box domain containing 1 | 196151 | 115183 | 123636 | 31, 10, 17 |
| 2 | WNT10A | Wnt family member 10A | 135925 | 115596 | 171450 | 37, 33, 14 |
| 2 | WNT6 | Wnt family member 6 | 115596 | 115592 | 135925 | 15, 27, 32 |
| 2 | WBP1 | WW domain binding protein 1 | 239779 | 274049 | 115275 | 26, 1, 24 |
| 2 | XDH | xanthine dehydrogenase | 158125 | 013016 | 277893 | 30, 16, 24 |
| 2 | XIRP2 | xin actin binding repeat containing 2 | 163092 | 136546 | 172318 | 25, 18, 15 |
| 2 | XRCC5 | X-ray repair cross complementing 5 | 079246 | 163449 | 144583 | 42, 36, 38 |
| 2 | YIPF4 | Yip1 domain family member 4 | 119820 | 091106 | 115760 | 4, 22, 34 |
| 2 | YPEL5 | yippee like 5 | 119801 | 171094 | 213626 | 29, 36, 38 |
| 2 | ZAP70 | zeta chain of T cell receptor associated protein kinase 70 | 115085 | 228486 | 075568 | 39, 5, 22 |
| 2 | ZFP36L2 | ZFP36 ring finger protein like 2 | 152518 | 162882 | 115970 | 1, 41, 11 |
| 2 | ZFAND2B | zinc finger AN1-type containing 2B | 158552 | 144567 | 115657 | 26, 41, 22 |
| 2 | ZC3H15 | zinc finger CCCH-type containing 15 | 065548 | 188738 | 281192 | 3, 34, 43 |
| 2 | ZC3H6 | zinc finger CCCH-type containing 6 | 188177 | 144161 | 169629 | |
| 2 | ZC3H8 | zinc finger CCCH-type containing 8 | 144161 | 144152 | 188177 | 11, 9, 31 |
| 2 | ZDBF2 | zinc finger DBF-type containing 2 | 204186 | 183671 | 279921 | 2, 13, 32 |
| 2 | ZEB2 | zinc finger E-box binding homeobox 2 | 169554 | 121964 | 121989 | 1, 8, 7 |
| 2 | ZNF142 | zinc finger protein 142 | 115568 | 115556 | 074582 | 43, 26, 27 |
| 2 | ZNF2 | zinc finger protein 2 | 275111 | 144026 | 233757 | 9, 42, 36 |
| 2 | ZNF385B | zinc finger protein 385B | 144331 | 187231 | 163510 | 14, 13, 38 |
| 2 | ZNF512 | zinc finger protein 512 | 243943 | 221843 | 176714 | 18, 39, 26 |
| 2 | ZNF513 | zinc finger protein 513 | 163795 | 115234 | 115241 | 24, 22, 33 |
| 2 | ZNF514 | zinc finger protein 514 | 144026 | 144029 | 275111 | 43, 24, 34 |
| 2 | ZNF638 | zinc finger protein 638 | 075292 | 124374 | 135636 | 18, 26, 4 |
| 2 | ZNF804A | zinc finger protein 804A | 170396 | 163002 | 188738 | 6, 8, 28 |
| 2 | ZRANB3 | zinc finger RANBP2-type containing 3 | 121988 | 115839 | 048991 | 1, 10, 23 |
| 2 | ZSWIM2 | zinc finger SWIM-type containing 2 | 163012 | 144369 | 064989 | 38, 8, |
| 3 | GBE1 | 1,4-alpha-glucan branching enzyme 1 | 114480 | 169855 | 175161 | 10, 45, 26 |
| 3 | HACL1 | 2-hydroxyacyl-CoA lyase 1 | 131373 | 206561 | 169814 | 10, 43, 38 |
| 3 | PXYLP1 | 2-phosphoxylose phosphatase 1 | 155893 | 175093 | 177311 | 39, 34, 2 |
| 3 | HACD2 | 3-hydroxyacyl-CoA dehydratase 2 | 206527 | 173175 | 065534 | 22, 23, 6 |
| 3 | BDH1 | 3-hydroxybutyrate dehydrogenase 1 | 161267 | 075711 | 145016 | 16, 1, 21 |
| 3 | OXSM | 3-oxoacyl-ACP synthase, mitochondrial | 151093 | 151092 | 179796 | |
| 3 | XRN1 | 5′-3′ exoribonuclease 1 | 114127 | 175066 | 175054 | 30, 24, 22 |
| 3 | ALAS1 | 5′-aminolevulinate synthase 1 | 023330 | 164087 | 239732 | 2, 5, 45 |
| 3 | AZI2 | 5-azacytidine induced 2 | 163512 | 187118 | 206559 | |
| 3 | HMCES | 5-hydroxymethylcytosine binding, ES cell specific | 183624 | 181789 | 184897 | 21, 41, 22 |
| 3 | HTR1F | 5-hydroxytryptamine receptor 1F | 179097 | 064835 | 163320 | 7, 28, 32 |
| 3 | HTR3C | 5-hydroxytryptamine receptor 3C | 178084 | 186090 | 186038 | 1, |
| 3 | HTR3D | 5-hydroxytryptamine receptor 3D | 186090 | 114770 | 178084 | |
| 3 | HTR3E | 5-hydroxytryptamine receptor 3E | 186038 | 178084 | 145191 | |
| 3 | NT5DC2 | 5′-nucleotidase domain containing 2 | 168268 | 010327 | 168273 | 3, 43, 40 |
| 3 | PFKFB4 | 6-phosphofructo-2-kinase/fructose-2,6-biphosphatase 4 | 114268 | 164054 | 145040 | 38, 35, 5 |
| 3 | OGG1 | 8-oxoguanine DNA glycosylase | 114026 | 156983 | 134072 | |
| 3 | ABHD14A- | ABHD14A-ACY1 readthrough | 114786 | 248487 | 243989 | 9, 3, 17 |
| | ACY1 | | | | | |
| 3 | ABHD10 | abhydrolase domain containing 10 | 144827 | 144824 | 144834 | 37, 31, 7 |
| 3 | ABHD14A | abhydrolase domain containing 14A | 248487 | 114779 | 114786 | 31, 29, 33 |
| 3 | ABHD14B | abhydrolase domain containing 14B | 114779 | 090097 | 248487 | 9, 21, 40 |
| 3 | ABHD5 | abhydrolase domain containing 5 | 011198 | 160746 | 173769 | 31, 32, 35 |
| 3 | ABHD6 | abhydrolase domain containing 6 | 163686 | 163687 | 163684 | |
| 3 | ABI3BP | ABI family member 3 binding protein | 154175 | 114354 | 081148 | 40, 45, 10 |
| 3 | ACAA1 | acetyl-CoA acyltransferase 1 | 060971 | 008226 | 172936 | 36, 19, 3 |
| 3 | ACPP | acid phosphatase, prostate | 014257 | 196353 | 138246 | 33, 26, 23 |
| 3 | ACTL6A | actin like 6A | 136518 | 114450 | 136522 | 10, 30, 38 |
| 3 | ARPC4 | actin related protein 2/3 complex subunit 4 | 241553 | 171148 | 250151 | 9, 13, 1 |
| 3 | ACTRT3 | actin related protein T3 | 184378 | 281137 | 085274 | 38, 9, 2 |
| 3 | ALCAM | activated leukocyte cell adhesion molecule | 170017 | 170044 | 114423 | 42, 24, 18 |
| 3 | ACVR2B | activin A receptor type 2B | 114739 | 093217 | 157036 | 11, 32, 12 |
| 3 | APEH | acylaminoacyl-peptide hydrolase | 164062 | 164061 | 173531 | 21, 41, 31 |
| 3 | ACAD11 | acyl-CoA dehydrogenase family member 11 | 240303 | 138246 | 274810 | 26, 44, 39 |
| 3 | ACAD9 | acyl-CoA dehydrogenase family member 9 | 177646 | 075785 | 114656 | 24, 36, 2 |
| 3 | ACOX2 | acyl-CoA oxidase 2 | 168306 | 168301 | 168309 | 43, 19, 26 |
| 3 | ADAMTS9 | ADAM metallopeptidase with thrombospondin type 1 motif 9 | 163638 | 163637 | 151276 | 11, 26, 18 |
| 3 | APPL1 | adaptor protein, phosphotyrosine interacting with PH domain and leucine zipper 1 | 157500 | 163666 | 239388 | 11, 22, 30 |
| 3 | AP2M1 | adaptor related protein complex 2 mu 1 subunit | 161203 | 161202 | 161204 | 18, 2, 43 |
| 3 | ADCY5 | adenylate cyclase 5 | 173175 | 121542 | 206527 | 1, 26, 31 |
| 3 | ADGRG7 | adhesion G protein-coupled receptor G7 | 144820 | 181458 | 114354 | 16, 2, 19 |
| 3 | AMIGO3 | adhesion molecule with Ig like domain 3 | 176020 | 164068 | 173540 | 34, 35, 27 |
| 3 | ADIPOQ | adiponectin, C1Q and collagen domain containing | 181092 | 163918 | 073849 | 1, 42, 26 |
| 3 | ARF4 | ADP ribosylation factor 4 | 168374 | 174840 | 174839 | 18, 11, 4 |
| 3 | ARL13B | ADP ribosylation factor like GTPase 13B | 169379 | 184500 | 178750 | 40, 30, 22 |
| 3 | ARL14 | ADP ribosylation factor like GTPase 14 | 179674 | 186432 | 163590 | 22, 4, 3 |
| 3 | ARL6 | ADP ribosylation factor like GTPase 6 | 113966 | 080224 | 080200 | 10, 38, |
| 3 | ARL6IP5 | ADP ribosylation factor like GTPase 6 interacting protein 5 | 144746 | 144744 | 163380 | |
| 3 | ARL8B | ADP ribosylation factor like GTPase 8B | 134108 | 134107 | 134109 | 39, 24, 26 |
| 3 | ADPRH | ADP-ribosylarginine hydrolase | 144843 | 121594 | 144837 | 22, 21, 26 |
| 3 | ALDH1L1 | aldehyde dehydrogenase 1 family member L1 | 144908 | 114544 | 163884 | 4, 17, 19 |
| 3 | ALG1L | ALG1, chitobiosyldiphosphodolichol beta-mannosyltransferase like | 189366 | 144909 | 114547 | 30, 24, 42 |
| 3 | ALG1L2 | ALG1, chitobiosyldiphosphodolichol beta-mannosyltransferase like 2 | 251287 | 170893 | 172752 | 24, 36, |
| 3 | ALG3 | ALG3, alpha-1,3-mannosyltransferase | 214160 | 145198 | 145194 | 21, 9, 27 |
| 3 | AHSG | alpha 2-HS glycoprotein | 145192 | 283149 | 090512 | 19, 5, 15 |
| 3 | A4GNT | alpha-1,4-N-acetylglucosaminyl transferase | 118017 | 158163 | 138231 | 37, 17, |
| 3 | ALS2CL | ALS2 C-terminal like | 178038 | 283473 | 181585 | 1, 9, 40 |
| 3 | ACY1 | aminoacylase 1 | 243989 | 114786 | 162244 | 9, 3, 17 |
| 3 | AMT | aminomethyl transferase | 145020 | 145022 | 283189 | 2, 31, 15 |
| 3 | ANAPC13 | anaphase promoting complex subunit 13 | 129055 | 114019 | 182923 | 4, 29, 31 |
| 3 | AMOTL2 | angiomotin like 2 | 114019 | 163785 | 129055 | 42, 12, 1 |
| 3 | AGTR1 | angiotensin II receptor type 1 | 144891 | 152977 | 153002 | 32, 26, 23 |
| 3 | ABTB1 | ankyrin repeat and BTB domain containing 1 | 114626 | 114631 | 074416 | 29, 31, 22 |
| 3 | ASB14 | ankyrin repeat and SOCS box containing 14 | 239388 | 157500 | 174844 | 18, 22, 25 |
| 3 | ANKUB1 | ankyrin repeat and ubiquitin domain containing 1 | 206199 | 114744 | 082996 | |
| 3 | ANKRD28 | ankyrin repeat domain 28 | 206560 | 169814 | 131386 | 37, 21, 40 |
| 3 | ANO10 | anoctamin 10 | 160746 | 163788 | 011198 | 23, |
| 3 | APOD | apolipoprotein D | 189058 | 184203 | 176945 | 26, 10, 1 |
| 3 | ACAP2 | ArfGAP with coiled-coil, ankyrin repeat and PH domains 2 | 114331 | 173950 | 184203 | 22, 11, 6 |
| 3 | RSRC1 | arginine and serine rich coiled-coil 1 | 174891 | 168779 | 178053 | 10, 7, 34 |
| 3 | ARGFX | arginine-fifty homeobox | 186103 | 051341 | 163833 | |
| 3 | ARIH2OS | ariadne homolog 2 opposite strand | 221883 | 178537 | 177479 | 2, 25, 24 |
| 3 | ARIH2 | ariadne RBR E3 ubiquitin protein ligase 2 | 177479 | 221883 | 178467 | 1, 25, 34 |
| 3 | ARMC8 | armadillo repeat containing 8 | 114098 | 138231 | 181322 | 10, 24, 39 |
| 3 | ACTR8 | ARP8 actin-related protein 8 homolog | 113812 | 056736 | 113811 | 6, 38, 24 |
| 3 | ARPC4-TTLL3 | ARPC4-TTLL3 readthrough | 250151 | 241553 | 214021 | |
| 3 | AADAC | arylacetamide deacetylase | 114771 | 197953 | 198829 | 2, 19, 1 |
| 3 | AADACL2 | arylacetamide deacetylase like 2 | 197953 | 152580 | 114771 | 44, 15, 35 |
| 3 | ASTE1 | asteroid homolog 1 (Drosophila) | 034533 | 280127 | 114670 | |
| 3 | ATXN7 | ataxin 7 | 163635 | 163634 | 163636 | 39, 11, 22 |
| 3 | ABCC5 | ATP binding cassette subfamily C member 5 | 114770 | 175193 | 186090 | 24, 18, 13 |
| 3 | ABCF3 | ATP binding cassette subfamily F member 3 | 161204 | 161203 | 145198 | 24, 2, 9 |
| 3 | ATP13A3 | ATPase13A3 | 133657 | 178732 | 145014 | 18, 4, 45 |
| 3 | ATP13A4 | ATPase13A4 | 127249 | 187527 | 198836 | 24, 31, 42 |
| 3 | ATP13A5 | ATPase13A5 | 187527 | 127252 | 127249 | 24, 10, 23 |
| 3 | ATP6V1A | ATPase H+ transporting V1 subunit A | 114573 | 121579 | 178075 | 11, 24, 45 |
| 3 | ATP1B3 | ATPase Na+/K+ transporting subunit beta 3 | 069849 | 114124 | 114126 | 37, 11, 9 |
| 3 | ATP11B | ATPase phospholipid transporting 11B (putative) | 058063 | 181449 | 043093 | 33, 39, 22 |
| 3 | ATP2B2 | ATPase plasma membrane Ca2+ transporting 2 | 157087 | 157020 | 132164 | 18, 8, 36 |
| 3 | ATP2C1 | ATPase secretory pathway Ca2+ transporting 1 | 017260 | 196455 | 280127 | 42, 24, 18 |
| 3 | ATRIP | ATR interacting protein | 164053 | 232112 | 213689 | 38, 13, 5 |
| 3 | ATRIP | ATR interacting protein | 282827 | 232112 | 213689 | 22, 41, 34 |
| 3 | ATR | ATR serine/threonine kinase | 175054 | 114127 | 120756 | 26, 10, 22 |
| 3 | ACKR2 | atypical chemokine receptor 2 | 144648 | 181061 | 273291 | |
| 3 | ACKR4 | atypical chemokine receptor 4 | 129048 | 274810 | 081307 | |
| 3 | ATG3 | autophagy related 3 | 144848 | 186265 | 138459 | 10, 4, 11 |
| 3 | ATG7 | autophagy related 7 | 197548 | 196639 | 144560 | 7, 30, 42 |
| 3 | BTLA | B and T lymphocyte associated | 186265 | 091972 | 144848 | 42, 30, 22 |
| 3 | BHLHE40 | basic helix-loop-helix family member e40 | 134107 | 150995 | 134108 | 4, 1, 40 |
| 3 | BSN | bassoon presynaptic cytomatrix protein | 164061 | 173402 | 164062 | 26, 8, 5 |
| 3 | BBX | BBX, HMG-box containing | 114439 | 138483 | 196776 | 41, 2, 22 |
| 3 | BCL6 | B-cell CLL/lymphoma 6 | 113916 | 198471 | 223401 | 1, 18, 26 |
| 3 | BFSP2 | beaded filament structural protein 2 | 170819 | 144868 | 091527 | 14, 10, |
| 3 | B3GALNT1 | beta-1,3-N-acetylgalactosaminyltransferase 1 (globoside blood group) | 169255 | 163590 | 169251 | |
| 3 | B4GALT4 | beta-1,4-galactosyltransferase 4 | 121578 | 114638 | 031081 | |
| 3 | BTD | biotinidase | 169814 | 131373 | 206560 | 17, 43, 38 |
| 3 | BOC | BOC cell adhesion associated, oncogene regulated | 144857 | 163608 | 206530 | 11, 1, 40 |
| 3 | BAP1 | BRCA1 associated protein 1 | 163930 | 114841 | 010318 | 26, 37, 33 |
| 3 | BRK1 | BRICK1, SCAR/WAVE actin nucleating complex subunit | 254999 | 163705 | 134086 | 7, 11, 30 |
| 3 | BRPF1 | bromodomain and PHD finger containing 1 | 156983 | 144550 | 114026 | 9, 43, 35 |
| 3 | BCHE | butyrylcholinesterase | 114200 | 121871 | 169064 | 10, 40, 43 |
| 3 | CELSR3 | cadherin EGF LAG seven-pass G-type receptor 3 | 008300 | 225697 | 213672 | 21, 40, 26 |
| 3 | CDHR4 | cadherin related family member 4 | 187492 | 176095 | 185614 | 20, 44, 38 |
| 3 | CADPS | calcium dependent secretion activator | 163618 | 153266 | 163630 | 26, 31, 14 |
| 3 | CASR | calcium sensing receptor | 036828 | 114013 | 283187 | 29, 17, 28 |
| 3 | CASR | calcium sensing receptor | 283187 | 036828 | 121552 | 29, 17, 28 |
| 3 | CACNA2D2 | calcium voltage-gated channel auxiliary subunit alpha2delta 2 | 007402 | 126062 | 088543 | 26, 38, 23 |
| 3 | CACNA2D3 | calcium voltage-gated channel auxiliary subunit alpha2delta 3 | 157445 | 113811 | 144771 | 31, 30, 10 |
| 3 | CACNA1D | calcium voltage-gated channel subunit alpha1 D | 157388 | 272886 | 016391 | 31, 16, 20 |
| 3 | CAMK1 | calcium/calmodulin dependent protein kinase I | 134072 | 114026 | 171148 | 22, 9, 27 |
| 3 | CAMK2N2 | calcium/calmodulin dependent protein kinase II inhibitor 2 | 163888 | 145194 | 175166 | 31, 28, 20 |
| 3 | CAPN7 | calpain 7 | 131375 | 131381 | 131370 | |
| 3 | CLSTN2 | calsyntenin 2 | 158258 | 163864 | 155890 | 23, 24, 33 |
| 3 | CAMKV | CaM kinase like vesicle associated | 164076 | 183763 | 164078 | 8, 38, 17 |
| 3 | ARPP21 | cAMP regulated phosphoprotein 21 | 172995 | 170248 | 144681 | 39, 8, 10 |
| 3 | CHST13 | carbohydrate sulfotransferase 13 | 180767 | 159650 | 180697 | 38, 17, 36 |
| 3 | CHST2 | carbohydrate sulfotransferase 2 | 175040 | 163714 | 181804 | 11, 31, 13 |
| 3 | CPA3 | carboxypeptidase A3 | 163751 | 153002 | 163754 | 7, 30, 39 |
| 3 | CPB1 | carboxypeptidase B1 | 153002 | 144891 | 163751 | 28, 4, 19 |
| 3 | CPN2 | carboxypeptidase N subunit 2 | 178772 | 114315 | 172061 | 19, 17, |
| 3 | CRTAP | cartilage associated protein | 170275 | 188167 | 173705 | 42, 18, 2 |
| 3 | CTNNB1 | catenin beta 1 | 168036 | 172888 | 168038 | 42, 24, 39 |
| 3 | CAMP | cathelicidin antimicrobial peptide | 164047 | 164045 | 164048 | 5, 7, 22 |
| 3 | CAV3 | caveolin 3 | 182533 | 125046 | 180914 | 25, 10, 33 |
| 3 | CBLB | Cbl proto-oncogene B | 114423 | 170017 | 138483 | 24, 42, 21 |
| 3 | CCR1 | C-C motif chemokine receptor 1 | 163823 | 183625 | 121807 | 5, 20, 28 |
| 3 | CCR2 | C-C motif chemokine receptor 2 | 121807 | 163823 | 160791 | 42, 36, 39 |
| 3 | CCR3 | C-C motif chemokine receptor 3 | 183625 | 173578 | 163823 | 10, 5, |
| 3 | CCR4 | C-C motif chemokine receptor 4 | 183813 | 206557 | 170266 | 1, 39, 5 |
| 3 | CCR5 | C-C motif chemokine receptor 5 (gene/pseudogene) | 160791 | 121807 | 121797 | 30, 22, 42 |
| 3 | CCR8 | C-C motif chemokine receptor 8 | 179934 | 168329 | 144659 | 5, |
| 3 | CCR9 | C-C motif chemokine receptor 9 | 173585 | 163818 | 163820 | 39, 16, 37 |
| 3 | CCRL2 | C-C motif chemokine receptor like 2 | 121797 | 160791 | 012223 | |
| 3 | CNBP | CCHC-type zinc finger nucleic acid binding protein | 169714 | 240682 | 181789 | 13, 30, 1 |
| 3 | CNOT10 | CCR4-NOT transcription complex subunit 10 | 182973 | 144635 | 206557 | 26, 17, 38 |
| 3 | CD200 | CD200 molecule | 091972 | 172139 | 186265 | 32, 1, 43 |
| 3 | CD200R1 | CD200 receptor 1 | 163606 | 206531 | 163607 | 42, 26, 7 |
| 3 | CD200R1L | CD200 receptor 1 like | 206531 | 091986 | 163606 | 38, |
| 3 | CD47 | CD47 molecule | 196776 | 114439 | 114446 | 13, 10, 18 |
| 3 | CD80 | CD80 molecule | 121594 | 113845 | 144843 | 41, 39, 23 |
| 3 | CD86 | CD86 molecule | 114013 | 145103 | 036828 | 5, 36, 30 |
| 3 | CD96 | CD96 molecule | 153283 | 177707 | 177494 | |
| 3 | CDV3 | CDV3 homolog | 091527 | 170819 | 163781 | 22, 6, 40 |
| 3 | CADM2 | cell adhesion molecule 2 | 175161 | 114480 | 206538 | 44, 8, |
| 3 | CHL1 | cell adhesion molecule L1 like | 134121 | 280530 | 134115 | |
| 3 | CIDEC | cell death inducing DFFA like effector c | 187288 | 156990 | 171135 | 1, 23, 7 |
| 3 | CDC25A | cell division cycle 25A | 164045 | 047849 | 164047 | 21, 25, 3 |
| 3 | CEP19 | centrosomal protein 19 | 174007 | 163964 | 180370 | 31, 15, 19 |
| 3 | CEP63 | centrosomal protein 63 | 182923 | 129055 | 154928 | 13, 40, 34 |
| 3 | CEP70 | centrosomal protein 70 | 114107 | 158220 | 158234 | 38, 39, 33 |
| 3 | CEP97 | centrosomal protein 97 | 182504 | 114391 | 144815 | 30, 7, 23 |
| 3 | CRBN | cereblon | 113851 | 281584 | 144455 | 29, 31, 7 |
| 3 | CP | ceruloplasmin | 047457 | 163755 | 163762 | 42, 19, 24 |
| 3 | CGGBP1 | CGG triplet repeat binding protein 1 | 163320 | 179097 | 175105 | 22, 29, 11 |
| 3 | CHMP2B | charged multivesicular body protein 2B | 083937 | 206538 | 064835 | 13, 45, 29 |
| 3 | CLCN2 | chloride voltage-gated channel 2 | 114859 | 175182 | 163882 | 34, 38, 16 |
| 3 | CCK | cholecystokinin | 187094 | 182606 | 157093 | 3, 32, 8 |
| 3 | CHDH | choline dehydrogenase | 016391 | 157388 | 056736 | 29, 1, 9 |
| 3 | CSPG5 | chondroitin sulfate proteoglycan 5 | 114646 | 163832 | 173473 | 2, 14, 8 |
| 3 | CHRD | chordin | 090539 | 090534 | 182580 | 1, 25, 8 |
| 3 | C3orf14 | chromosome 3 open reading frame 14 | 114405 | 144724 | 153266 | 28, 26, 30 |
| 3 | C3orf18 | chromosome 3 open reading frame 18 | 088543 | 007402 | 114735 | 29, 31, 15 |
| 3 | C3orf20 | chromosome 3 open reading frame 20 | 131379 | 154781 | 154783 | 38, 8, |
| 3 | C3orf22 | chromosome 3 open reading frame 22 | 180697 | 180767 | 283374 | 38, 8, |
| 3 | C3orf30 | chromosome 3 open reading frame 30 | 163424 | 144847 | 251012 | 38, |
| 3 | C3orf33 | chromosome 3 open reading frame 33 | 174928 | 114805 | 169359 | 45, 2, 9 |
| 3 | C3orf36 | chromosome 3 open reading frame 36 | 221972 | 154917 | 174640 | 36, 17, |
| 3 | C3orf38 | chromosome 3 open reading frame 38 | 179021 | 175105 | 283434 | |
| 3 | C3orf49 | Chromosome 3 open reading frame 49 | 163632 | 188817 | 163634 | 10, 44, 20 |
| 3 | C3orf52 | chromosome 3 open reading frame 52 | 114529 | 176040 | 174500 | 39, 22, 20 |
| 3 | C3orf58 | chromosome 3 open reading frame 58 | 181744 | 181804 | 152952 | 24, 23, 11 |
| 3 | C3orf62 | chromosome 3 open reading frame 62 | 188315 | 173421 | 114316 | 11, 26, 35 |
| 3 | C3orf67 | chromosome 3 open reading frame 67 | 163689 | 198643 | 189283 | 10, 22, 38 |
| 3 | C3orf70 | chromosome 3 open reading frame 70 | 187068 | 156931 | 113790 | 31, 42, 32 |
| 3 | C3orf80 | chromosome 3 open reading frame 80 | 180044 | 168811 | 248710 | 8, 20, 44 |
| 3 | C3orf84 | chromosome 3 open reading frame 84 | 236980 | 185909 | 173421 | 38, |
| 3 | CFAP100 | cilia and flagella associated protein 100 | 163885 | 163884 | 070476 | 38, 44, 20 |
| 3 | CFAP44 | cilia and flagella associated protein 44 | 206530 | 144857 | 283241 | |
| 3 | CMTM6 | CKLF like MARVEL transmembrane domain containing 6 | 091317 | 153551 | 144635 | 30, 4, 43 |
| 3 | CMTM7 | CKLF like MARVEL transmembrane domain containing 7 | 153551 | 170293 | 091317 | 18, 35, 43 |
| 3 | CMTM8 | CKLF like MARVEL transmembrane domain containing 8 | 170293 | 152642 | 153551 | 28, 10, 18 |
| 3 | CLRN1 | clarin 1 | 163646 | 260234 | 144893 | 14, 25, |
| 3 | CLDN1 | claudin 1 | 163347 | 090530 | 113946 | 4, 42, 40 |
| 3 | CLDN11 | claudin 11 | 013297 | 136603 | 013293 | 23, 8, |
| 3 | CLDN16 | claudin 16 | 113946 | 163347 | 198398 | 17, 39, 37 |
| 3 | CLDN18 | claudin 18 | 066405 | 168875 | 158163 | 37, 3, 20 |
| 3 | CLDND1 | claudin domain containing 1 | 080822 | 231861 | 080819 | 8, 13, 30 |
| 3 | CMSS1 | cms1 ribosomal small subunit homolog (yeast) | 184220 | 144810 | 168386 | 6, 25, 5 |
| 3 | COPB2 | coatomer protein complex subunit beta 2 | 184432 | 233701 | 114113 | 24, 42, 39 |
| 3 | COPG1 | coatomer protein complex subunit gamma 1 | 181789 | 169714 | 183624 | 43, 24, 1 |
| 3 | CCDC12 | coiled-coil domain containing 12 | 160799 | 160801 | 160796 | 44, 5, 32 |
| 3 | CCDC13 | coiled-coil domain containing 13 | 244607 | 157119 | 280571 | 14, 38, 2 |
| 3 | CCDC14 | coiled-coil domain containing 14 | 175455 | 065534 | 065371 | 39, 22, 13 |
| 3 | CCDC174 | coiled-coil domain containing 174 | 154781 | 144596 | 131379 | 3, 27, 43 |
| 3 | CCDC191 | coiled-coil domain containing 191 | 163617 | 184307 | 151576 | 1, 31, 6 |
| 3 | CCDC36 | coiled-coil domain containing 36 | 173421 | 236980 | 188315 | 18, 38, 13 |
| 3 | CCDC39 | coiled-coil domain containing 39 | 145075 | 163728 | 114416 | |
| 3 | CCDC50 | coiled-coil domain containing 50 | 152492 | 188958 | 114279 | 1, 11, 24 |
| 3 | CCDC51 | coiled-coil domain containing 51 | 164051 | 164050 | 232112 | 9, 40, 21 |
| 3 | CCDC54 | coiled-coil domain containing 54 | 138483 | 114423 | 114439 | 38, 44, |
| 3 | CCDC58 | coiled-coil domain containing 58 | 160124 | 121552 | 114023 | 4, 3, 30 |
| 3 | CCDC66 | coiled-coil domain containing 66 | 180376 | 187672 | 163946 | 4, 30, 12 |
| 3 | CCDC71 | coiled-coil domain containing 71 | 177352 | 172037 | 185909 | 13, 5, 40 |
| 3 | CCDC80 | coiled-coil domain containing 80 | 091986 | 138459 | 206531 | 1, 10, 6 |
| 3 | CHCHD4 | coiled-coil-helix-coiled-coil-helix domain containing 4 | 163528 | 154764 | 170876 | 1, 9, 30 |
| 3 | CHCHD6 | coiled-coil-helix-coiled-coil-helix domain containing 6 | 159685 | 197763 | 114554 | 3, 10, 14 |
| 3 | COLQ | collagen like tail subunit of asymmetric acetylcholinesterase | 206561 | 144597 | 131373 | 10, |
| 3 | COL6A5 | collagen type VI alpha 5 chain | 172752 | 251287 | 206384 | 10, 38, 20 |
| 3 | COL6A6 | collagen type VI alpha 6 chain | 206384 | 172752 | 196455 | 29, 18, 28 |
| 3 | COL7A1 | collagen type VII alpha 1 chain | 114270 | 145040 | 010256 | 18, 4, 13 |
| 3 | COL8A1 | collagen type VIII alpha 1 chain | 144810 | 057019 | 184220 | 6, 1, 45 |
| 3 | COMMD2 | COMM domain containing 2 | 114744 | 018408 | 206199 | 30, 34, 43 |
| 3 | CNTN3 | contactin 3 | 113805 | 121440 | 172969 | 11, 31, 45 |
| 3 | CNTN4 | contactin 4 | 144619 | 134115 | 091181 | 29, 45, 38 |
| 3 | CNTN6 | contactin 6 | 134115 | 134121 | 144619 | 11, 8, 2 |
| 3 | CPNE4 | copine 4 | 196353 | 114686 | 014257 | 33, 38, |
| 3 | CPNE9 | copine family member 9 | 144550 | 163719 | 156983 | 26, 31, 28 |
| 3 | CPOX | coproporphyrinogen oxidase | 080819 | 080822 | 154165 | 19, 28, 5 |
| 3 | COX17 | COX17, cytochrome c oxidase copper chaperone | 138495 | 121577 | 183833 | 31, 15, 29 |
| 3 | CRYBG3 | crystallin beta-gamma domain containing 3 | 080200 | 113966 | 170854 | 26, 24, 16 |
| 3 | CRYGS | crystallin gamma S | 213139 | 058866 | 113838 | 14, 1, 45 |
| 3 | CTDSPL | CTD small phosphatase like | 144677 | 144668 | 136059 | 15, 16, 1 |
| 3 | CLEC3B | C-type lectin domain family 3 member B | 163815 | 075914 | 163814 | 1, 36, 6 |
| 3 | CDCP1 | CUB domain containing protein 1 | 163814 | 163815 | 249992 | 40, 13, 16 |
| 3 | CAND2 | cullin associated and neddylation dissociated 2 (putative) | 144712 | 088726 | 144713 | 40, 11, 31 |
| 3 | CX3CR1 | C-X3-C motif chemokine receptor 1 | 168329 | 168334 | 179934 | 36, 8, 17 |
| 3 | CMC1 | C-X9-C motif containing 1 | 187118 | 163508 | 163512 | 34, 27, 17 |
| 3 | CXCR6 | C-X-C motif chemokine receptor 6 | 172215 | 163820 | 173578 | 32, 36, 44 |
| 3 | CCNL1 | cyclin L1 | 163660 | 197980 | 197415 | |
| 3 | CSTA | cystatin A | 121552 | 283187 | 160124 | 30, 24, 31 |
| 3 | CSRNP1 | cysteine and serine rich nuclear protein 1 | 144655 | 168026 | 168334 | 6, 37, 1 |
| 3 | CRELD1 | cysteine rich with EGF like domains 1 | 163703 | 163702 | 163704 | 1, 43, 9 |
| 3 | CYB561D2 | cytochrome b561 family member D2 | 114395 | 114388 | 272104 | 22, 31, 27 |
| 3 | CYP8B1 | cytochrome P450 family 8 subfamily B member 1 | 180432 | 240747 | 182983 | |
| 3 | CISH | cytokine inducible SH2 containing protein | 114737 | 114735 | 114738 | 27, 32, 7 |
| 3 | CLASP2 | cytoplasmic linker associated protein 2 | 163539 | 153560 | 170248 | 8, 11, 22 |
| 3 | DALRD3 | DALR anticodon binding domain containing 3 | 178149 | 178252 | 178057 | 41, 38, 13 |
| 3 | DZIP1L | DAZ interacting zinc finger protein 1 like | 158163 | 066405 | 118017 | 1, 30, 7 |
| 3 | DZIP3 | DAZ interacting zinc finger protein 3 | 198919 | 280684 | 163515 | 42, 26, 31 |
| 3 | DCAF1 | DDB1 and CUL4 associated factor 1 | 145041 | 259956 | 164080 | 31, 34, 40 |
| 3 | DHX36 | DEAH-box helicase 36 | 174953 | 114790 | 174948 | 11, 12, 35 |
| 3 | DBR1 | debranching RNA lariats 1 | 138231 | 118017 | 114098 | 7, 35, 24 |
| 3 | DCP1A | decapping mRNA 1A | 272886 | 163931 | 157388 | 30, 43, 44 |
| 3 | D0CK3 | dedicator of cytokinesis 3 | 088538 | 114738 | 145050 | 8, 40, 14 |
| 3 | DCUN1D1 | defective in cullin neddylation 1 domain containing 1 | 043093 | 058063 | 078070 | 38, 22, 2 |
| 3 | DAZL | deleted in azoospermia like | 092345 | 131378 | 154822 | 38, 10, 25 |
| 3 | DLEC1 | deleted in lung and esophageal cancer 1 | 008226 | 187091 | 060971 | 20, 40, 38 |
| 3 | DTX3L | deltex E3 ubiquitin ligase 3L | 163840 | 138496 | 173200 | 29, 42, 21 |
| 3 | DENND6A | DENN domain containing 6A | 174839 | 168374 | 163681 | 29, 11, 24 |
| 3 | DNASE1L3 | deoxyribonuclease 1 like 3 | 163687 | 136068 | 163686 | 36, 30, 17 |
| 3 | DPPA2 | developmental pluripotency associated 2 | 163530 | 241224 | 121570 | 10, 38, 12 |
| 3 | DPPA4 | developmental pluripotency associated 4 | 121570 | 163530 | 177707 | 12, 39, 38 |
| 3 | DHX30 | DExH-box helicase 30 | 132153 | 173473 | 047849 | 35, 31, 38 |
| 3 | DGKG | diacylglycerol kinase gamma | 058866 | 244405 | 213139 | 2, 5, 31 |
| 3 | DHFR2 | dihydrofolate reductase 2 | 178700 | 178750 | 178694 | 36, 26, 31 |
| 3 | DPH3 | diphthamide biosynthesis 3 | 154813 | 131386 | 154814 | 31, 45, 43 |
| 3 | DCBLD2 | discoidin, CUB and LCCL domain containing 2 | 057019 | 064225 | 144810 | 45, 43, 40 |
| 3 | DLG1 | discs large MAGUK scaffold protein 1 | 075711 | 163975 | 161267 | 22, 11, 24 |
| 3 | DVL3 | dishevelled segment polarity protein 3 | 161202 | 145191 | 161203 | 40, 18, 16 |
| 3 | DIRC2 | disrupted in renal carcinema 2 | 138463 | 169087 | 082684 | 13, 9, 23 |
| 3 | POLQ | DNA polymerase theta | 051341 | 145087 | 186103 | 18, 11, 22 |
| 3 | DNAJB11 | DnaJ heat shock protein family (Hsp40) member B11 | 090520 | 113838 | 283149 | 40, 11, 15 |
| 3 | DNAJB8 | DnaJ heat shock protein family (Hsp40) member B8 | 179407 | 132394 | 179348 | 38, 5, 8 |
| 3 | DNAJC13 | DnaJ heat shock protein family (Hsp40) member C13 | 138246 | 014257 | 240303 | 24, 29, 31 |
| 3 | DNAJC19 | DnaJ heat shock protein family (Hsp40) member C19 | 205981 | 114416 | 181449 | 33, 31, 34 |
| 3 | DRD3 | dopamine receptor D3 | 151577 | 151576 | 174255 | 38, |
| 3 | DCLK3 | doublecortin like kinase 3 | 163673 | 144681 | 168016 | 38, 10, 45 |
| 3 | DUSP7 | dual specificity phosphatase 7 | 164086 | 162244 | 164087 | 9, 21, 45 |
| 3 | DNAH1 | dynein axonemal heavy chain 1 | 114841 | 168237 | 163930 | 22, 32, 20 |
| 3 | DNAH12 | dynein axonemal heavy chain 12 | 174844 | 239388 | 174840 | 30, 10, 20 |
| 3 | DYNC1LI1 | dynein cytoplasmic 1 light intermediate chain 1 | 144635 | 091317 | 182973 | 43, 31, 15 |
| 3 | DAG1 | dystroglycan 1 | 173402 | 145029 | 164061 | 25, 9, 32 |
| 3 | ENTPD3 | ectonucleoside triphosphate diphosphohydrolase 3 | 168032 | 114784 | 188846 | 28, 26, 31 |
| 3 | EFCC1 | EF-hand and coiled-coil domain containing 1 | 114654 | 187695 | 169704 | 27, 36, 6 |
| 3 | EFCAB12 | EF-hand calcium binding domain 12 | 172771 | 184897 | 129071 | 42, 38, 24 |
| 3 | EFHB | EF-hand domain family member B | 163576 | 183960 | 144566 | 17, 44, |
| 3 | EOGT | EGF domain specific O-linked N-acetylglucosamine transferase | 163378 | 163377 | 144747 | 11, 45, 42 |
| 3 | ERC2 | ELKS/RAB6-interacting/CAST family member 2 | 187672 | 114251 | 180376 | 31, 26, 8 |
| 3 | EAF1 | ELL associated factor 1 | 144597 | 206562 | 206561 | |
| 3 | EAF2 | ELL associated factor 2 | 145088 | 173226 | 163406 | 17, 41, 5 |
| 3 | ELP6 | elongator acetyltransferase complex subunit 6 | 163832 | 114650 | 114646 | 5, 27, 1 |
| 3 | EBLN2 | endogenous Bornavirus-like nucleoprotein 2 | 255423 | 163605 | 121440 | 36, 19, 22 |
| 3 | ECE2 | endothelin converting enzyme 2 | 145194 | 214160 | 163888 | 9, 10, 31 |
| 3 | EHHADH | enoyl-CoA hydratase and 3-hydroxyacyl CoA dehydrogenase | 113790 | 187068 | 073803 | 42, 19, 24 |
| 3 | EOMES | eomesodermin | 163508 | 033867 | 187118 | 30, 12, 8 |
| 3 | EPHA3 | EPH receptor A3 | 044524 | 283434 | 184500 | 42, 33, 8 |
| 3 | EPHA6 | EPH receptor A6 | 080224 | 269028 | 113966 | 38, 33, 8 |
| 3 | EPHB1 | EPH receptor B1 | 154928 | 182923 | 174611 | 8, 38, 23 |
| 3 | EPHB3 | EPH receptor B3 | 182580 | 090539 | 177383 | 34, 16, 18 |
| 3 | ECT2 | epithelial cell transforming 2 | 114346 | 144959 | 144962 | 24, 3, 13 |
| 3 | EPM2AIP1 | EPM2A interacting protein 1 | 178567 | 168016 | 076242 | 18, 11, 42 |
| 3 | EDEM1 | ER degradation enhancing alpha-mannosidase like protein 1 | 134109 | 134108 | 196277 | 22, 39, 9 |
| 3 | EMC3 | ER membrane protein complex subunit 3 | 125037 | 163704 | 144554 | 30, 2, 29 |
| 3 | ETV5 | ETS variant 5 | 244405 | 136527 | 058866 | 35, 42, 38 |
| 3 | EEFSEC | eukaryotic elongation factor, selenocysteine-tRNA specific | 132394 | 175792 | 179407 | 34, 21, 9 |
| 3 | EIF1B | eukaryotic translation initiation factor 1B | 114784 | 170011 | 168032 | 4, 28, 34 |
| 3 | EIF2A | eukaryotic translation initiation factor 2A | 144895 | 120742 | 198843 | 4, 10, 37 |
| 3 | EIF2B5 | eukaryotic translation initiation factor 2B subunit epsilon | 145191 | 186038 | 161202 | 24, 3, 35 |
| 3 | EIF4G1 | eukaryotic translation initiation factor 4 gamma 1 | 114867 | 175166 | 175182 | 23, 3, 18 |
| 3 | EIF4A2 | eukaryotic translation initiation factor 4A2 | 156976 | 113889 | 163918 | 39, 13, 31 |
| 3 | EIF4E3 | eukaryotic translation initiation factor 4E family member 3 | 163412 | 114861 | 170837 | 26, 1, 25 |
| 3 | EIF5A2 | eukaryotic translation initiation factor 5A2 | 163577 | 163584 | 163581 | 31, 11, 34 |
| 3 | EXOG | exo/endonuclease G | 157036 | 114739 | 183873 | 26, 18, 40 |
| 3 | EXOSC7 | exosome component 7 | 075914 | 163812 | 163815 | 29, 41, 11 |
| 3 | ESYT3 | extended synaptotagmin 3 | 158220 | 158186 | 114107 | 38, 17, 32 |
| 3 | FAM107A | family with sequence similarity 107 member A | 168309 | 168306 | 198643 | 8, 14, 15 |
| 3 | FAM131A | family with sequence similarity 131 member A | 175182 | 114867 | 114859 | 6, 28, 8 |
| 3 | FAM162A | family with sequence similarity 162 member A | 114023 | 160124 | 196981 | 29, 11, 15 |
| 3 | FAM19A1 | family with sequence similarity 19 member A1, C-C motif chemokine like | 183662 | 172340 | 163377 | 17, 8, |
| 3 | FAM19A4 | family with sequence similarity 19 member A4, C-C motif chemokine like | 163377 | 183662 | 163378 | 2, 38, 14 |
| 3 | FAM198A | family with sequence similarity 198 member A | 144649 | 182983 | 144647 | 1, 36, 44 |
| 3 | FAM208A | family with sequence similarity 208 member A | 163946 | 180376 | 163947 | 29, 22, 26 |
| 3 | FAM212A | family with sequence similarity 212 member A | 185614 | 187492 | 182179 | 41, 22, 20 |
| 3 | FAM3D | family with sequence similarity 3 member D | 198643 | 168309 | 163689 | 37, 16, 18 |
| 3 | FAM43A | family with sequence similarity 43 member A | 185112 | 041802 | 173950 | 24, 45, 36 |
| 3 | FANCD2OS | FANCD2 opposite strand | 163705 | 144554 | 254999 | |
| 3 | FANCD2 | Fanconi anemia complementation group D2 | 144554 | 125037 | 163705 | 37, 45, 6 |
| 3 | FAIM | Fas apoptotic inhibitory molecule | 158234 | 114107 | 051382 | 13, 14, 3 |
| 3 | FBXL2 | F-box and leucine rich repeat protein 2 | 153558 | 173705 | 153560 | 8, 38, 26 |
| 3 | FBXW12 | F-box and WD repeat domain containing 12 | 164049 | 229453 | 164050 | 38, |
| 3 | FBXO40 | F-box protein 40 | 163833 | 186103 | 180353 | 25, 15, 19 |
| 3 | FBXO45 | F-box protein 45 | 174013 | 185798 | 174004 | 3, 7, 22 |
| 3 | FRMD4B | FERM domain containing 4B | 114541 | 163380 | 187098 | 42, 1, 43 |
| 3 | FETUB | fetuin B | 090512 | 145192 | 113905 | 19, 24, 42 |
| 3 | FEZF2 | FEZ family zinc finger 2 | 153266 | 114405 | 163618 | 14, 8, 12 |
| 3 | FGF12 | fibroblast growth factor 12 | 114279 | 152492 | 180611 | 10, 11, 8 |
| 3 | FNDC3B | fibronectin type III domain containing 3B | 075420 | 186329 | 121853 | 11, 1, 23 |
| 3 | FBLN2 | fibulin 2 | 163520 | 163517 | 154764 | 38, 1, 42 |
| 3 | FILIP1L | filamin A interacting protein 1 like | 168386 | 184220 | 036054 | 45, 18, 25 |
| 3 | FLNB | filamin B | 136068 | 163681 | 163687 | 43, 9, 19 |
| 3 | FXR1 | FMR1 autosomal homolog 1 | 114416 | 145075 | 205981 | 39, 15, 13 |
| 3 | FSTL1 | follistatin like 1 | 163430 | 163428 | 065518 | 42, 18, 12 |
| 3 | FOXL2 | forkhead box L2 | 183770 | 051382 | 206262 | 27, 44, 9 |
| 3 | FOXP1 | forkhead box P1 | 114861 | 242120 | 163412 | 22, 45, 30 |
| 3 | FYTTD1 | forty-two-three domain containing 1 | 122068 | 145016 | 186001 | 38, 22, 39 |
| 3 | FOXL2NB | FOXL2 neighbor | 206262 | 183770 | 206260 | |
| 3 | FHIT | fragile histidine triad | 189283 | 163689 | 144724 | 23, 10, 37 |
| 3 | FRG2C | FSHD region gene 2 family member C | 172969 | 113805 | 227124 | |
| 3 | FYCO1 | FYVE and coiled-coil domain containing 1 | 163820 | 173585 | 172215 | |
| 3 | FGD5 | FYVE, RhoGEF and PH domain containing 5 | 154783 | 131379 | 177463 | 43, 36, 15 |
| 3 | GFM1 | G elongation factor mitochondrial 1 | 168827 | 178053 | 079257 | 42, 24, 26 |
| 3 | GNL3 | G protein nucleolar 3 | 163938 | 163939 | 016864 | 3, 45, 7 |
| 3 | GNAI2 | G protein subunit alpha i2 | 114353 | 188338 | 012171 | 41, 5, 35 |
| 3 | GNAT1 | G protein subunit alpha transducin 1 | 114349 | 001617 | 188338 | 14, 25, 8 |
| 3 | GNB4 | G protein subunit beta 4 | 114450 | 171109 | 136518 | 11, 43, 34 |
| 3 | GPR149 | G protein-coupled receptor 149 | 174948 | 174953 | 196549 | 8, |
| 3 | GPR15 | G protein-coupled receptor 15 | 154165 | 080819 | 064225 | 5, 39, 19 |
| 3 | GPR156 | G protein-coupled receptor 156 | 175697 | 082701 | 163428 | 13, 33, 38 |
| 3 | GPR160 | G protein-coupled receptor 160 | 173890 | 008952 | 173889 | |
| 3 | GPR171 | G protein-coupled receptor 171 | 174946 | 144893 | 174944 | 30, 45, 39 |
| 3 | GPR27 | G protein-coupled receptor 27 | 170837 | 163412 | 163421 | 27, 5, 28 |
| 3 | GPR62 | G protein-coupled receptor 62 | 180929 | 041880 | 090097 | 27, 8, 33 |
| 3 | GPR87 | G protein-coupled receptor 87 | 138271 | 174944 | 181631 | 4, 15, 2 |
| 3 | GRK7 | G protein-coupled receptor kinase 7 | 114124 | 114125 | 069849 | |
| 3 | GLB1 | galactosidase beta 1 | 170266 | 183813 | 188167 | 7, 13, 36 |
| 3 | GABRR3 | gamma-aminobutyric acid type A receptor rho3 subunit (gene/pseudogene) | 183185 | 170854 | 213439 | 2, 14, |
| 3 | GATA2 | GATA binding protein 2 | 179348 | 179407 | 163902 | 32, 33, 11 |
| 3 | GMPPB | GDP-mannose pyrophosphorylase B | 173540 | 176020 | 176095 | 42, 32, 35 |
| 3 | GMNC | geminin coiled-coil domain containing | 205835 | 196083 | 188729 | 42, 44, 27 |
| 3 | GTF2E1 | general transcription factor HE subunit 1 | 153767 | 144840 | 145087 | 39, 24, 38 |
| 3 | GCSAM | germinal center associated signaling and motility | 174500 | 114529 | 172139 | 21, 22, 5 |
| 3 | GHRL | ghrelin and obestatin prepropeptide | 157017 | 272410 | 157020 | 5, 10, 37 |
| 3 | GXYLT2 | glucoside xylosyltransferase 2 | 172986 | 144736 | 163605 | 31, 29, 18 |
| 3 | GADL1 | glutamate decarboxylase like 1 | 144644 | 163513 | 163527 | 42, 5, |
| 3 | GRM2 | glutamate metabotropic receptor 2 | 164082 | 164081 | 214686 | 8, 42, 14 |
| 3 | GRM7 | glutamate metabotropic receptor 7 | 196277 | 134109 | 071282 | 42, 8, 14 |
| 3 | GRIP2 | glutamate receptor interacting protein 2 | 144596 | 131389 | 154781 | 26, 6, 3 |
| 3 | ERICH6 | glutamate rich 6 | 163645 | 198843 | 181788 | 38, 33, |
| 3 | QRICH1 | glutamine rich 1 | 198218 | 178035 | 172053 | 39, 38, 42 |
| 3 | QARS | glutaminyl-tRNA synthetase | 172053 | 198218 | 172046 | 39, 36, 45 |
| 3 | GPX1 | glutathione peroxidase 1 | 233276 | 114316 | 067560 | 43, 1, 34 |
| 3 | GLYCTK | glycerate kinase | 168237 | 164091 | 114841 | 24, 19, 5 |
| 3 | GK5 | glycerol kinase 5 (putative) | 175066 | 114126 | 114127 | |
| 3 | GPD1L | glycerol-3-phosphate dehydrogenase 1-like | 152642 | 197385 | 170293 | 42, 24, 15 |
| 3 | GSK3B | glycogen synthase kinase 3 beta | 082701 | 144852 | 175697 | 29, 10, 22 |
| 3 | GYG1 | glycogenin 1 | 163754 | 163751 | 071794 | |
| 3 | GP9 | glycoprotein IX platelet | 169704 | 114654 | 172780 | 15, 14, 20 |
| 3 | GP5 | glycoprotein V platelet | 178732 | 172061 | 133657 | 7, |
| 3 | GLT8D1 | glycosyltransferase 8 domain containing 1 | 016864 | 163938 | 114902 | 31, 38, 2 |
| 3 | GOLIM4 | golgi integral membrane protein 4 | 173905 | 163536 | 085276 | 16, 45, 13 |
| 3 | GORASP1 | golgi reassembly stacking protein 1 | 114745 | 114742 | 168026 | 39, 27, 13 |
| 3 | GOLGA4 | golgin A4 | 144674 | 093167 | 144668 | 37, 24, 19 |
| 3 | GOLGB1 | golgin B1 | 173230 | 180353 | 173226 | 18, 40, 29 |
| 3 | GRAMD1C | GRAM domain containing 1C | 178075 | 114573 | 184307 | 42, 38, 31 |
| 3 | GAP43 | growth associated protein 43 | 172020 | 279380 | 185565 | 26, 10, 8 |
| 3 | GHSR | growth hormone secretagogue receptor | 121853 | 075420 | 121858 | 20, |
| 3 | GTPBP8 | GTP binding protein 8 (putative) | 163607 | 163606 | 163608 | 31, 41, 42 |
| 3 | GMPS | guanine monophosphate synthase | 163655 | 169359 | 169282 | 13, 3, 5 |
| 3 | GUCA1C | guanylate cyclase activator 1C | 138472 | 163519 | 114487 | 14, 10, 5 |
| 3 | H1FOO | H1 histone family member O, oocyte specific | 178804 | 163914 | 004399 | 38, |
| 3 | H1FX | H1 histone family member X | 184897 | 183624 | 172771 | 20, 33, 6 |
| 3 | HEG1 | heart development protein with EGF like domains 1 | 173706 | 173702 | 221955 | 1, 40, 10 |
| 3 | HHATL | hedgehog acyltransferase-like | 010282 | 157119 | 280571 | 25, 24, 14 |
| 3 | HLTF | helicase like transcription factor | 071794 | 163754 | 163755 | 2, 26, 9 |
| 3 | HCLS1 | hematopoietic cell-specific Lyn substrate 1 | 180353 | 163833 | 173230 | 21, 36, 30 |
| 3 | HEMK1 | HemK methyltransferase family member 1 | 114735 | 088543 | 114737 | 22, 34, 17 |
| 3 | HHLA2 | HERV-H LTR-associating 2 | 114455 | 114446 | 144821 | 16, 42, 3 |
| 3 | HES1 | hes family bHLH transcription factor 1 | 114315 | 198836 | 178772 | 19, 29, 26 |
| 3 | HESX1 | HESX homeobox 1 | 163666 | 144730 | 157500 | 27, 44, 16 |
| 3 | HIGD1A | HIG1 hypoxia inducible domain family member 1A | 181061 | 280571 | 144648 | |
| 3 | HRH1 | histamine receptor H1 | 196639 | 157103 | 197548 | 10, 26, 13 |
| 3 | HRG | histidine rich glycoprotein | 113905 | 090512 | 113889 | 19, 23, 17 |
| 3 | HDAC11 | histone deacetylase 11 | 163517 | 132182 | 163520 | 8, 43, 26 |
| 3 | HGD | homogentisate 1,2-dioxygenase | 113924 | 065518 | 144840 | 19, 17, 16 |
| 3 | HPS3 | HPS3, biogenesis of lysosomal organelles complex 2 subunit 1 | 163755 | 071794 | 047457 | 24, 22, 9 |
| 3 | HRASLS | HRAS like suppressor | 127252 | 180611 | 187527 | 27, 31, 38 |
| 3 | HSPBAP1 | HSPB1 associated protein 1 | 169087 | 173193 | 138463 | 6, 9, 32 |
| 3 | HYAL1 | hyaluronoglucosaminidase 1 | 114378 | 243477 | 068001 | 36, 9, 2 |
| 3 | HYAL2 | hyaluronoglucosaminidase 2 | 068001 | 114378 | 114383 | 1, 36, 45 |
| 3 | HYAL3 | hyaluronoglucosaminidase 3 | 186792 | 214706 | 243477 | |
| 3 | ILDR1 | immunoglobulin like domain containing receptor 1 | 145103 | 163406 | 114013 | 26, 27, 4 |
| 3 | IGSF10 | immunoglobulin superfamily member 10 | 152580 | 169313 | 197953 | 20, |
| 3 | IGSF11 | immunoglobulin superfamily member 11 | 144847 | 185565 | 163424 | 26, 38, 8 |
| 3 | IMPDH2 | inosine monophosphate dehydrogenase 2 | 178035 | 178057 | 198218 | 41, 21, 27 |
| 3 | ITPR1 | inositol 1,4,5-trisphosphate receptor type 1 | 150995 | 170364 | 134107 | 29, 26, 31 |
| 3 | IP6K1 | inositol hexakisphosphate kinase 1 | 176095 | 173540 | 187492 | 43, 11, 26 |
| 3 | IP6K2 | inositol hexakisphosphate kinase 2 | 068745 | 213672 | 114302 | 39, 29, 44 |
| 3 | IGF2BP2 | insulin like growth factor 2 mRNA binding protein 2 | 073792 | 163904 | 136527 | 12, 11, 18 |
| 3 | ITGA9 | integrin subunit alpha 9 | 144668 | 144674 | 144677 | 2, 15, 40 |
| 3 | ITGB5 | integrin subunit beta 5 | 082781 | 114491 | 173702 | 27, 24, 34 |
| 3 | ITIH1 | inter-alpha-trypsin inhibitor heavy chain 1 | 055957 | 114904 | 162267 | 19, 36, 23 |
| 3 | ITIH3 | inter-alpha-trypsin inhibitor heavy chain 3 | 162267 | 055957 | 055955 | 19, 15, 36 |
| 3 | ITIH4 | inter-alpha-trypsin inhibitor heavy chain family member 4 | 055955 | 162267 | 243696 | 19, 4, 26 |
| 3 | IFRD2 | interferon related developmental regulator 2 | 214706 | 179564 | 186792 | 9, 37, 41 |
| 3 | IL1RAP | interleukin 1 receptor accessory protein | 196083 | 198398 | 205835 | 18, 32, 7 |
| 3 | IRAK2 | interleukin 1 receptor associated kinase 2 | 134070 | 134086 | 157014 | 9, 4, 7 |
| 3 | IL12A | interleukin 12A | 168811 | 151967 | 180044 | 36, 16, 44 |
| 3 | IL17RB | interleukin 17 receptor B | 056736 | 016391 | 113812 | 9, 17, 26 |
| 3 | IL17RC | interleukin 17 receptor C | 163702 | 163701 | 163703 | 33, 29, 9 |
| 3 | IL17RD | interleukin 17 receptor D | 144730 | 186451 | 163666 | 12, 11, 38 |
| 3 | IL17RE | interleukin 17 receptor E | 163701 | 171135 | 163702 | 37, 39, 16 |
| 3 | IL20RB | interleukin 20 receptor subunit beta | 174564 | 158092 | 168875 | 30, 9, 18 |
| 3 | IL5RA | interleukin 5 receptor subunit alpha | 091181 | 144619 | 072756 | 42, 35, 33 |
| 3 | IMPG2 | interphotoreceptor matrix proteoglycan 2 | 081148 | 154175 | 138468 | 14, 25, 22 |
| 3 | IFT122 | intraflagellar transport 122 | 163913 | 129071 | 163914 | 38, 42, 24 |
| 3 | IFT57 | intraflagellar transport 57 | 114446 | 196776 | 114455 | 22, 1, 31 |
| 3 | IFT80 | intraflagellar transport 80 | 068885 | 248710 | 113810 | 39, 38, |
| 3 | IQSEC1 | IQ motif and Sec7 domain 1 | 144711 | 144713 | 132182 | 29, 14, 18 |
| 3 | IQCB1 | IQ motif containing B1 | 173226 | 173230 | 145088 | 9, 7, 22 |
| 3 | IQCF1 | IQ motif containing F1 | 173389 | 214681 | 114767 | 38, 8, |
| 3 | IQCF2 | IQ motif containing F2 | 184345 | 229972 | 214681 | 38, 8, |
| 3 | IQCF3 | IQ motif containing F3 | 229972 | 214686 | 184345 | 38, 20, |
| 3 | IQCF5 | IQ motif containing F5 | 214681 | 184345 | 173389 | 38, |
| 3 | IQCF6 | IQ motif containing F6 | 214686 | 164082 | 229972 | 38, |
| 3 | IQCG | IQ motif containing G | 114473 | 186001 | 182899 | |
| 3 | IQCJ | IQ motif containing J | 214216 | 118855 | 283154 | 17, 8, 38 |
| 3 | IQCJ-SCHIP1 | IQCJ-SCHIP1 readthrough | 283154 | 214216 | 283245 | |
| 3 | ISY1 | ISY1 splicing factor homolog | 240682 | 261796 | 169714 | 41, 35, 27 |
| 3 | ISY1-RAB43 | ISY1-RAB43 readthrough | 261796 | 172780 | 240682 | 41, 35, 27 |
| 3 | JAGN1 | jagunal homolog 1 | 171135 | 187288 | 163701 | 9, 1, 6 |
| 3 | KALRN | kalirin, RhoGEF kinase | 160145 | 065371 | 114491 | 3, 34, 29 |
| 3 | KPNA1 | karyopherin subunit alpha 1 | 114030 | 196981 | 138496 | 45, 9, 24 |
| 3 | KPNA4 | karyopherin subunit alpha 4 | 186432 | 213186 | 179674 | 18, 23, 2 |
| 3 | KLHDC8B | kelch domain containing 8B | 185909 | 177352 | 236980 | 1, 29, 2 |
| 3 | KLHL18 | kelch like family member 18 | 114648 | 088727 | 076201 | 26, 25, 22 |
| 3 | KLHL24 | kelch like family member 24 | 114796 | 172578 | 163872 | 40, 35, 24 |
| 3 | KLHL40 | kelch like family member 40 | 157119 | 114853 | 010282 | 24, 15, 25 |
| 3 | KLHL6 | kelch like family member 6 | 172578 | 176597 | 114796 | 22, 21, 39 |
| 3 | KBTBD12 | kelch repeat and BTB domain containing 12 | 187715 | 074416 | 058262 | 25, 38, 33 |
| 3 | KBTBD8 | kelch repeat and BTB domain containing 8 | 163376 | 144749 | 172340 | 22, 10, 39 |
| 3 | KIAA1143 | KIAA1143 | 163807 | 186446 | 163808 | 1, 42, 21 |
| 3 | KIAA1257 | KIAA1257 | 114656 | 177646 | 187695 | 38, 30, 24 |
| 3 | KIAA1524 | KIAA1524 | 163507 | 144821 | 280684 | 23, 39, 13 |
| 3 | KIF15 | kinesin family member 15 | 163808 | 163807 | 169964 | 12, 3, 34 |
| 3 | KIF9 | kinesin family member 9 | 088727 | 181555 | 114648 | 38, 17, 40 |
| 3 | KNG1 | kininogen 1 | 113889 | 113905 | 156976 | 19, 17, 23 |
| 3 | KRBOX1 | KRAB box domain containing 1 | 240747 | 273291 | 180432 | 38, 34, 15 |
| 3 | KLF15 | Kruppel like factor 15 | 163884 | 144908 | 163885 | 26, 17, 33 |
| 3 | KY | kyphoscoliosis peptidase | 174611 | 154928 | 073711 | 18, 2, 37 |
| 3 | LTF | lactotransferrin | 012223 | 121797 | 163825 | 42, 5, 30 |
| 3 | LAMB2 | laminin subunit beta 2 | 172037 | 172046 | 177352 | 1, 26, 6 |
| 3 | LSG1 | large 60S subunit nuclear export GTPase 1 | 041802 | 145014 | 185112 | 24, 3, 11 |
| 3 | LXN | latexin | 079257 | 168827 | 118849 | 5, 1, 43 |
| 3 | LMOD3 | leiomodin 3 | 163380 | 144746 | 114541 | 15, 25, 26 |
| 3 | LMLN | leishmanolysin like peptidase | 185621 | 182899 | 280530 | 43, 11, 42 |
| 3 | LRRC15 | leucine rich repeat containing 15 | 172061 | 178772 | 178732 | 6, 15, 24 |
| 3 | LRRC2 | leucine rich repeat containing 2 | 163827 | 163825 | 241186 | 25, 11, 15 |
| 3 | LRRC31 | leucine rich repeat containing 31 | 114248 | 188306 | 187033 | 37, 40, 16 |
| 3 | LRRC34 | leucine rich repeat containing 34 | 171757 | 085274 | 188306 | 31, 5, 38 |
| 3 | LRRC3B | leucine rich repeat containing 3B | 179796 | 151093 | 163491 | |
| 3 | LRRC58 | leucine rich repeat containing 58 | 163428 | 175697 | 163430 | 2, 1, 29 |
| 3 | LRRN1 | leucine rich repeat neuronal 1 | 175928 | 144455 | 170364 | |
| 3 | LRCH3 | leucine rich repeats and calponin homology domain containing 3 | 186001 | 122068 | 114473 | 33, 14, 12 |
| 3 | LRIG1 | leucine rich repeats and immunoglobulin like domains 1 | 144749 | 144741 | 163376 | 42, 11, 15 |
| 3 | LRRIQ4 | leucine rich repeats and IQ motif containing 4 | 188306 | 171757 | 114248 | 10, 20, 38 |
| 3 | LRTM1 | leucine rich repeats and transmembrane domains 1 | 144771 | 157445 | 114251 | 14, 28, 20 |
| 3 | LSMEM2 | leucine rich single-pass membrane protein 2 | 179564 | 012171 | 214706 | 33, 22, 15 |
| 3 | LZTFL1 | leucine zipper transcription factor like 1 | 163818 | 163817 | 173585 | |
| 3 | LEKR1 | leucine, glutamate and lysine rich 1 | 197980 | 163659 | 163660 | 34, 42, 4 |
| 3 | LARS2 | leucyl-tRNA synthetase 2, mitochondrial | 011376 | 249992 | 144791 | 43, 42, 41 |
| 3 | LNP1 | leukemia NUP98 fusion partner 1 | 206535 | 154174 | 181458 | 35, 38, 14 |
| 3 | LMCD1 | LIM and cysteine rich domains 1 | 071282 | 196277 | 125046 | 29, 43, 15 |
| 3 | LPP | LIM domain containing preferred translocation partner in lipoma | 145012 | 223401 | 188001 | 22, 42, 25 |
| 3 | LIMD1 | LIM domains containing 1 | 144791 | 011376 | 211456 | 2, 40, 37 |
| 3 | LSAMP | limbic system-associated membrane protein | 185565 | 172020 | 144847 | 34, 14, 22 |
| 3 | LIPH | lipase H | 163898 | 163900 | 163904 | 16, 3, 24 |
| 3 | LHFPL4 | lipoma HMGIC fusion partner-like 4 | 156959 | 168137 | 163719 | 14, 28, 8 |
| 3 | LINC00694 | Long intergenic non-protein coding RNA 694 | 225873 | 179152 | 185219 | 32, 38, |
| 3 | LRRFIP2 | LRR binding FLU interacting protein 2 | 093167 | 076242 | 144674 | 26, 4, 36 |
| 3 | LSM3 | LSM3 homolog, U6 small nuclear RNA and mRNA degradation associated | 170860 | 154767 | 131389 | 11, 29, 43 |
| 3 | KAT2B | lysine acetyltransferase 2B | 114166 | 183977 | 129810 | 26, 45, 11 |
| 3 | LAMP3 | lysosomal associated membrane protein 3 | 078081 | 078070 | 053524 | 39, 13, 30 |
| 3 | LYZL4 | lysozyme like 4 | 157093 | 187094 | 114812 | 10, 38, 14 |
| 3 | MB21D2 | Mab-21 domain containing 2 | 180611 | 114279 | 127252 | 17, 32, 24 |
| 3 | MST1 | macrophage stimulating 1 | 173531 | 164062 | 164068 | |
| 3 | MST1R | macrophage stimulating 1 receptor | 164078 | 164076 | 164077 | 16, 37, 33 |
| 3 | MAGEF1 | MAGE family member F1 | 177383 | 182580 | 156931 | 41, 21, 35 |
| 3 | MFSD1 | major facilitator superfamily domain containing 1 | 118855 | 118849 | 214216 | 2, 36, 24 |
| 3 | MKRN2 | makorin ring finger protein 2 | 075975 | 225526 | 132155 | 7, 38, 11 |
| 3 | MSL2 | male-specific lethal 2 homolog (Drosophila) | 174579 | 073711 | 114054 | 39, 24, 40 |
| 3 | MASP1 | mannan binding lectin serine peptidase 1 | 127241 | 175077 | 136514 | 44, 42, 19 |
| 3 | MAP6D1 | MAP6 domain containing 1 | 180834 | 163872 | 175193 | 8, 29, 22 |
| 3 | MCF2L2 | MCF.2 cell line derived transforming sequence-like 2 | 053524 | 078081 | 176597 | 24, 18, 1 |
| 3 | MECOM | MDS1 and EV11 complex locus | 085276 | 173905 | 281137 | |
| 3 | MED12L | mediator complex subunit 12 like | 144893 | 163646 | 174946 | |
| 3 | MITF | melanogenesis associated transcription factor | 187098 | 114541 | 242120 | 35, 1, 15 |
| 3 | MELTF | melanotransferrin | 163975 | 119227 | 075711 | 3, 35, 28 |
| 3 | MAGI1 | membrane associated guanylate kinase, WW and PDZ domain containing 1 | 151276 | 163638 | 144741 | 36, 23, 31 |
| 3 | MME | membrane metalloendopeptidase | 196549 | 174948 | 240045 | 22, 17, 6 |
| 3 | MANF | mesencephalic astrocyte derived neurotrophic factor | 145050 | 088538 | 259956 | 28, 43, 35 |
| 3 | MBD4 | methyl-CpG binding domain 4, DNA glycosylase | 129071 | 172771 | 163913 | 13, 39, 22 |
| 3 | MCCC1 | methylcrotonoyl-CoA carboxylase 1 | 078070 | 043093 | 078081 | 13, 17, 10 |
| 3 | METTL6 | methyltransferase like 6 | 206562 | 131370 | 144597 | 30, 43, 26 |
| 3 | MAP4 | microtubule associated protein 4 | 047849 | 132153 | 164045 | 21, 26, 14 |
| 3 | MCM2 | minichromosome maintenance complex component 2 | 073111 | 163870 | 114631 | 21, 39, 24 |
| 3 | MRPL3 | mitochondrial ribosomal protein L3 | 114686 | 198585 | 196353 | 2, 37, 41 |
| 3 | MRPL47 | mitochondrial ribosomal protein L47 | 136522 | 136518 | 136521 | 24, 29, 2 |
| 3 | MRPS22 | mitochondrial ribosomal protein S22 | 175110 | 206260 | 184814 | |
| 3 | MRPS25 | mitochondrial ribosomal protein S25 | 131368 | 177463 | 131381 | |
| 3 | MFN1 | mitofusin 1 | 171109 | 121864 | 114450 | 42, 35, 3 |
| 3 | MAP3K13 | mitogen-activated protein kinase kinase kinase 13 | 073803 | 113790 | 163900 | |
| 3 | MAPKAPK3 | mitogen-activated protein kinase-activated protein kinase 3 | 114738 | 114737 | 088538 | 40, 5, 3 |
| 3 | MKRN20S | MKRN2 opposite strand | 225526 | 154743 | 075975 | 44, |
| 3 | MON1A | MON1 homolog A, secretory trafficking associated | 164077 | 164078 | 004534 | 9, 2, 43 |
| 3 | MGLL | monoglyceride lipase | 074416 | 114626 | 187715 | 1, 28, 43 |
| 3 | MORC1 | MORO family CW-type zinc finger 1 | 114487 | 138472 | 241224 | 10, 38, |
| 3 | MTRNR2L12 | MT-RNR2-like 12 | 269028 | 178694 | 080224 | 14, 35, 38 |
| 3 | MUC13 | mucin 13, cell surface associated | 173702 | 082781 | 173706 | 16, 37, 42 |
| 3 | MUC20 | mucin 20, cell surface associated | 176945 | 189058 | 145113 | 13, 24, 17 |
| 3 | MUC4 | mucin 4, cell surface associated | 145113 | 176945 | 061938 | 30, 24, 37 |
| 3 | MRAS | muscle RAS oncogene homolog | 158186 | 181322 | 158220 | 26, 1, 2 |
| 3 | MBNL1 | muscleblind like splicing regulator 1 | 152601 | 198829 | 169860 | 45, 22, 34 |
| 3 | MUSTN1 | musculoskeletal, embryonic nuclear protein 1 | 272573 | 243696 | 248592 | 25, 1, 15 |
| 3 | MLH1 | mutL homolog 1 | 076242 | 178567 | 093167 | 13, 10, 21 |
| 3 | MINA | MYC induced nuclear antigen | 170854 | 080200 | 183185 | 11, 4, 1 |
| 3 | MAATS1 | MYCBP associated and testis expressed 1 | 183833 | 138495 | 144852 | 38, 26, 31 |
| 3 | MOBP | myelin-associated oligodendrocyte basic protein | 168314 | 168028 | 170011 | 8, 26, 17 |
| 3 | MYD88 | myeloid differentiation primary response 88 | 172936 | 060971 | 172939 | 5, 36, 24 |
| 3 | MLF1 | myeloid leukemia factor 1 | 178053 | 174891 | 168827 | 4, 38, 30 |
| 3 | MYNN | myoneurin | 085274 | 184378 | 171757 | 42, 30, 31 |
| 3 | MYH15 | myosin heavy chain 15 | 144821 | 114455 | 163507 | 10, 42, 44 |
| 3 | MYL3 | myosin light chain 3 | 160808 | 178055 | 160801 | |
| 3 | MYLK | myosin light chain kinase | 065534 | 206527 | 175455 | 44, 13, 33 |
| 3 | MYRIP | myosin VIIA and Rab interacting protein | 170011 | 168314 | 114784 | 11, 45, 2 |
| 3 | MTMR14 | myotubularin related protein 14 | 163719 | 156959 | 144550 | 36, 5, 38 |
| 3 | NAA50 | N(alpha)-acetyltransferase 50, NatE catalytic subunit | 121579 | 176542 | 114573 | 42, 24, 22 |
| 3 | NAALADL2 | N-acetylated alpha-linked acidic dipeptidase like 2 | 177694 | 169760 | 177565 | |
| 3 | NAT6 | N-acetyltransferase 6 | 243477 | 186792 | 114378 | |
| 3 | NDUFAF3 | NADH: ubiquinone oxidoreductase complex assembly factor 3 | 178057 | 178149 | 178035 | 41, 38, 26 |
| 3 | NDUFB4 | NADH: ubiquinone oxidoreductase subunit B4 | 065518 | 163430 | 113924 | 7, 2, 25 |
| 3 | NDUFB5 | NADH: ubiquinone oxidoreductase subunit B5 | 136521 | 136522 | 058056 | 34, 4, 29 |
| 3 | NKTR | natural killer cell triggering receptor | 114857 | 008324 | 114853 | 22, 29, 40 |
| 3 | NCBP2-AS2 | NCBP2 antisense RNA 2 (head to head) | 270170 | 114503 | 119227 | |
| 3 | NCK1 | NCK adaptor protein 1 | 158092 | 168917 | 174564 | 43, 39, 18 |
| 3 | NCKIPSD | NCK interacting protein with SH3 domain | 213672 | 008300 | 068745 | 21, 3, 43 |
| 3 | NECTIN3 | nectin cell adhesion molecule 3 | 177707 | 121570 | 153283 | 32, 38, |
| 3 | NRROS | negative regulator of reactive oxygen species | 174004 | 174013 | 163964 | 5, 32, 21 |
| 3 | NPHP3 | nephrocystin 3 | 113971 | 081307 | 144868 | 11, 36, 45 |
| 3 | NXPE3 | neurexophilin and PC-esterase domain family member 3 | 144815 | 182504 | 144802 | 37, 13, 2 |
| 3 | NBEAL2 | neurobeachin like 2 | 160796 | 160799 | 181555 | 22, 5, 26 |
| 3 | NLGN1 | neuroligin 1 | 169760 | 144962 | 177694 | |
| 3 | NCEH1 | neutral cholesterol ester hydrolase 1 | 144959 | 121858 | 114346 | 24, 4, 42 |
| 3 | NKIRAS1 | NFKB inhibitor interacting Ras like 1 | 197885 | 170142 | 174748 | 15, 8, 38 |
| 3 | NFKBIZ | NFKB inhibitor zeta | 144802 | 144815 | 170044 | 40, 37, 20 |
| 3 | NGLY1 | N-glycanase 1 | 151092 | 077097 | 151093 | 34, 5, 11 |
| 3 | NICN1 | nicolin 1 | 145029 | 283189 | 173402 | 29, 1, 30 |
| 3 | NMNAT3 | nicotinamide nucleotide adenylyltransferase 3 | 163864 | 114115 | 158258 | 14, 38, 18 |
| 3 | NEK10 | NIMA related kinase 10 | 163491 | 179796 | 033867 | |
| 3 | NEK11 | NIMA related kinase 11 | 114670 | 034533 | 198585 | 10, 31, 5 |
| 3 | NEK4 | NIMA related kinase 4 | 114904 | 114902 | 055957 | 38, 1, 13 |
| 3 | NISCH | nischarin | 010322 | 114854 | 010327 | 21, 38, 33 |
| 3 | NIT2 | nitrilase family member 2 | 114021 | 036054 | 154174 | 40, 4, 17 |
| 3 | NMD3 | NMD3 ribosome export adaptor | 169251 | 169255 | 196542 | 4, 24, 45 |
| 3 | NME9 | NME/NM23 family member 9 | 181322 | 114098 | 158186 | 40, 30, 4 |
| 3 | NME6 | NME/NM23 nucleoside diphosphate kinase 6 | 172113 | 164048 | 229453 | 21, 45, 25 |
| 3 | NSUN3 | NOP2/Sun RNA methyltransferase family member 3 | 178694 | 178700 | 269028 | |
| 3 | NPHP3- | NPHP3-ACAD11 readthrough (NMD candidate) | 274810 | 240303 | 129048 | 11, 36, 45 |
| | ACAD11 | | | | | |
| 3 | NPRL2 | NPR2-like, GATOR1 complex subunit | 114388 | 004838 | 114395 | 39, 36, 16 |
| 3 | NCBP2 | nuclear cap binding protein subunit 2 | 114503 | 119231 | 270170 | 13, 45, 36 |
| 3 | NR1D2 | nuclear receptor subfamily 1 group D member 2 | 174738 | 174748 | 151090 | 14, 4, 6 |
| 3 | NR1I2 | nuclear receptor subfamily 1 group I member 2 | 144852 | 183833 | 082701 | 19, 7, 37 |
| 3 | NR2C2 | nuclear receptor subfamily 2 group C member 2 | 177463 | 154783 | 131368 | 31, 42, 22 |
| 3 | NEPRO | nucleolus and neural progenitor protein | 163608 | 163607 | 144857 | 42, 36, 1 |
| 3 | NUP210 | nucleoporin 210 | 132182 | 144711 | 163517 | 22, 20, 33 |
| 3 | NUDT16 | nudix hydrolase 16 | 198585 | 114670 | 114686 | 26, 17, 40 |
| 3 | OR5AC1 | olfactory receptor family 5 subfamily AC member 1 (gene/pseudogene) | 213439 | 183185 | 196578 | |
| 3 | OR5AC2 | olfactory receptor family 5 subfamily AC member 2 | 196578 | 213439 | 231192 | |
| 3 | OR5H1 | olfactory receptor family 5 subfamily H member 1 | 231192 | 196578 | 236032 | |
| 3 | OR5H14 | olfactory receptor family 5 subfamily H member 14 | 236032 | 231192 | 233412 | |
| 3 | OR5H15 | olfactory receptor family 5 subfamily H member 15 | 233412 | 236032 | 230301 | |
| 3 | OR5H2 | olfactory receptor family 5 subfamily H member 2 | 197938 | 230301 | 232535 | |
| 3 | OR5H6 | olfactory receptor family 5 subfamily H member 6 (gene/pseudogene) | 230301 | 233412 | 197938 | |
| 3 | OR5H8 | olfactory receptor family 5 subfamily H member 8 (gene/pseudogene) | 232535 | 197938 | 196098 | |
| 3 | OR5K1 | olfactory receptor family 5 subfamily K member 1 | 232382 | 206536 | 231861 | |
| 3 | OR5K2 | olfactory receptor family 5 subfamily K member 2 | 231861 | 232382 | 080822 | |
| 3 | OR5K3 | olfactory receptor family 5 subfamily K member 3 | 206536 | 196098 | 232382 | |
| 3 | OR5K4 | olfactory receptor family 5 subfamily K member 4 | 196098 | 232535 | 206536 | |
| 3 | OPA1 | OPA1, mitochondrial dynamin like GTPase | 198836 | 127249 | 114315 | 7, 40, 6 |
| 3 | OSTN | osteocrin | 188729 | 205835 | 188958 | |
| 3 | OTOL1 | otolin 1 | 182447 | 196542 | 090402 | |
| 3 | OXSR1 | oxidative stress responsive 1 | 172939 | 172936 | 172940 | 26, 11, 12 |
| 3 | OXNAD1 | oxidoreductase NAD binding domain containing 1 | 154814 | 154813 | 131378 | 39, 40, 31 |
| 3 | OSBPL10 | oxysterol binding protein like 10 | 144645 | 163527 | 197385 | 44, |
| 3 | OSBPL11 | oxysterol binding protein like 11 | 144909 | 114520 | 189366 | 18, 4, 22 |
| 3 | OXTR | oxytocin receptor | 180914 | 182533 | 070950 | 35, 17, 44 |
| 3 | PAK2 | p21 (RAC1) activated kinase 2 | 180370 | 174007 | 119231 | 30, 29, 4 |
| 3 | PTH1R | parathyroid hormone 1 receptor | 160801 | 160808 | 160799 | 6, 26, 15 |
| 3 | PDZRN3 | PDZ domain containing ring finger 3 | 121440 | 255423 | 113805 | 18, 38, 1 |
| 3 | PTX3 | pentraxin 3 | 163661 | 197415 | 174899 | 7, 10, 6 |
| 3 | PEX5L | peroxisomal biogenesis factor 5 like | 114757 | 058056 | 163728 | 29, 11, 8 |
| 3 | PPARG | peroxisome proliferator activated receptor gamma | 132170 | 157150 | 154743 | 32, 16, 1 |
| 3 | PCNP | PEST proteolytic signal containing nuclear protein | 081154 | 174173 | 066422 | 26, 43, 7 |
| 3 | PHF7 | PHD finger protein 7 | 010318 | 163930 | 010319 | 38, 34, 22 |
| 3 | PCYT1A | phosphate cytidylyltransferase 1, choline, alpha | 161217 | 163959 | 272741 | 18, 1, 24 |
| 3 | PIGX | phosphatidylinositol glycan anchor biosynthesis class X | 163964 | 174004 | 174007 | 20, 22, 16 |
| 3 | PIGZ | phosphatidylinositol glycan anchor biosynthesis class Z | 119227 | 270170 | 163975 | 31, 35, 18 |
| 3 | PLCXD2 | phosphatidylinositol specific phospholipase C X domain containing 2 | 240891 | 177494 | 144824 | 32, 8, 39 |
| 3 | PIK3CA | phosphatidylinositol-4,5-bisphosphate 3-kinase catalytic subunit alpha | 121879 | 172667 | 171121 | |
| 3 | PIK3CB | phosphatidylinositol-4,5-bisphosphate 3-kinase catalytic subunit beta | 051382 | 158234 | 183770 | 7, 31, 45 |
| 3 | PDE12 | phosphodiesterase 12 | 174840 | 174844 | 168374 | 11, 29, 24 |
| 3 | PIK3R4 | phosphoinositide-3-kinase regulatory subunit 4 | 196455 | 206384 | 017260 | 18, 29, 26 |
| 3 | PLA1A | phospholipase A1 member A | 144837 | 144843 | 121577 | 29, 30, 35 |
| 3 | PLCD1 | phospholipase C delta 1 | 187091 | 136059 | 008226 | 39, 38, 36 |
| 3 | PLCH1 | phospholipase C eta 1 | 114805 | 240045 | 174928 | 38, 12, 4 |
| 3 | PLCL2 | phospholipase C like 2 | 154822 | 092345 | 131374 | |
| 3 | PLD1 | phospholipase D1 | 075651 | 154310 | 186329 | |
| 3 | PLSCR1 | phospholipid scramblase 1 | 188313 | 163746 | 231213 | 23, 37, 4 |
| 3 | PLSCR2 | phospholipid scramblase 2 | 163746 | 114698 | 188313 | 38, |
| 3 | PLSCR4 | phospholipid scramblase 4 | 114698 | 152952 | 163746 | 11, 18, 1 |
| 3 | PLSCR5 | phospholipid scramblase family member 5 | 231213 | 188313 | 174963 | 32, |
| 3 | PLS1 | plastin 1 | 120756 | 175054 | 144935 | 42, 3, 16 |
| 3 | PHLDB2 | pleckstrin homology like domain family B member 2 | 144824 | 240891 | 144827 | 11, 32, 26 |
| 3 | PLXNA1 | plexin A1 | 114554 | 159685 | 163870 | 18, 4, 6 |
| 3 | PLXNB1 | plexin B1 | 164050 | 164049 | 164051 | 18, 42, 40 |
| 3 | PLXND1 | plexin D1 | 004399 | 178804 | 172765 | 26, 1, 7 |
| 3 | POC1A | POC1 centriolar protein A | 164087 | 164086 | 023330 | 32, 23, 37 |
| 3 | PODXL2 | podocalyxin like 2 | 114631 | 073111 | 114626 | 26, 14, 3 |
| 3 | PARP14 | poly(ADP-ribose) polymerase family member 14 | 173193 | 173200 | 169087 | 1, 18, 22 |
| 3 | PARP15 | poly(ADP-ribose) polymerase family member 15 | 173200 | 163840 | 173193 | 39, 22, 36 |
| 3 | PARP3 | poly(ADP-ribose) polymerase family member 3 | 041880 | 114767 | 180929 | 41, 2, 14 |
| 3 | PARP9 | poly(ADP-ribose) polymerase family member 9 | 138496 | 114030 | 163840 | 13, 4, 23 |
| 3 | PCBP4 | poly(rC) binding protein 4 | 090097 | 180929 | 114779 | 14, 8, 45 |
| 3 | PBRM1 | polybromo 1 | 163939 | 168273 | 163938 | 22, |
| 3 | PHC3 | polyhomeotic homolog 3 | 173889 | 173890 | 163558 | 11, 30, 29 |
| 3 | GALNT15 | polypeptide N-acetylgalactosaminyltransferase 15 | 131386 | 206560 | 154813 | 42, 10, 43 |
| 3 | POPDC2 | popeye domain containing 2 | 121577 | 144837 | 138495 | 10, |
| 3 | KCNMB2 | potassium calcium-activated channel subfamily M regulatory beta subunit 2 | 197584 | 177565 | 275163 | 26, 28, 10 |
| 3 | KCNMB3 | potassium calcium-activated channel subfamily M regulatory beta subunit 3 | 171121 | 121879 | 121864 | 22, 3, 15 |
| 3 | KCTD6 | potassium channel tetramerization domain containing 6 | 168301 | 168291 | 168306 | 9, 34, 11 |
| 3 | KCNAB1 | potassium voltage-gated channel subfamily A member regulatory beta subunit 1 | 169282 | 163655 | 114850 | 26, 8, 39 |
| 3 | KCNH8 | potassium voltage-gated channel subfamily H member 8 | 183960 | 182568 | 163576 | 36, 4, 8 |
| 3 | POU1F1 | POU class 1 homeobox 1 | 064835 | 083937 | 179097 | 31, |
| 3 | PQLC2L | PQ loop repeat containing 2 like | 174899 | 163661 | 168779 | 10, 18, 44 |
| 3 | PARL | presenilin associated rhomboid like | 175193 | 180834 | 114770 | 18, 34, 27 |
| 3 | PRICKLE2 | prickle planar cell polarity protein 2 | 163637 | 163636 | 163638 | |
| 3 | PCOLCE2 | procollagen C-endopeptidase enhancer 2 | 163710 | 144935 | 188582 | 31, 10, 1 |
| 3 | PLOD2 | procollagen-lysine,2-oxoglutarate 5-dioxygenase 2 | 152952 | 181744 | 114698 | 40, 45, 11 |
| 3 | PFN2 | profilin 2 | 070087 | 082996 | 196428 | 8, 31, 25 |
| 3 | PAQR9 | progestin and adipoQ receptor family member 9 | 188582 | 163710 | 163714 | 38, |
| 3 | PDCD10 | programmed cell death 10 | 114209 | 174776 | 163536 | 34, 4, 24 |
| 3 | PDCD6IP | programmed cell death 6 interacting protein | 170248 | 163539 | 172995 | 11, 45, 7 |
| 3 | PROK2 | prokineticin 2 | 163421 | 170837 | 163602 | 38, 45, 7 |
| 3 | PRR23A | proline rich 23A | 206260 | 206262 | 175110 | 38, |
| 3 | PRR23B | proline rich 23B | 184814 | 175110 | 233701 | 38, |
| 3 | PRR23C | proline rich 23C | 233701 | 184814 | 184432 | 38, |
| 3 | PRRT3 | proline rich transmembrane protein 3 | 163704 | 163703 | 125037 | 26, 31, 34 |
| 3 | P3H2 | prolyl 3-hydroxylase 2 | 090530 | 073282 | 163347 | 2, 17, 33 |
| 3 | P4HTM | prolyl 4-hydroxylase, transmembrane | 178467 | 177479 | 178252 | 9, 26, 31 |
| 3 | PCCB | propionyl-CoA carboxylase beta subunit | 114054 | 174579 | 118007 | 31, 3, 2 |
| 3 | PRSS42 | protease, serine 42 | 178055 | 188086 | 160808 | 25, 15, 28 |
| 3 | PRSS45 | protease, serine 45 | 188086 | 261603 | 178055 | 33, 39, |
| 3 | PRSS46 | protease, serine 46 | 261603 | 206549 | 188086 | 38, |
| 3 | PRSS50 | protease, serine 50 | 206549 | 181585 | 261603 | 38, 28, 44 |
| 3 | PSMD2 | proteasome 26S subunit, non-ATPase 2 | 175166 | 163888 | 114867 | 35, 38, 34 |
| 3 | PSMD6 | proteasome 26S subunit, non-ATPase 6 | 163636 | 163635 | 163637 | 18, 41, 23 |
| 3 | PDIA5 | protein disulfide isomerase family A member 5 | 065485 | 082684 | 121542 | 32, 6, 9 |
| 3 | PRKCD | protein kinase C delta | 163932 | 163933 | 163931 | 3, 21, 26 |
| 3 | PRKCI | protein kinase C iota | 163558 | 173889 | 136603 | 31, 4, 7 |
| 3 | PRKAR2A | protein kinase cAMP-dependent type II regulatory subunit alpha | 114302 | 068745 | 178537 | 21, 38, 3 |
| 3 | POGLUT1 | protein O-glucosyltransferase 1 | 163389 | 176142 | 113845 | 11, 29, 18 |
| 3 | POMGNT2 | protein O-linked mannose N-acetylglucosaminyltransferase 2 (beta 1,4-) | 144647 | 144649 | 163788 | |
| 3 | PPP1R2 | protein phosphatase 1 regulatory inhibitor subunit 2 | 184203 | 114331 | 189058 | 29, 38, 23 |
| 3 | PPP2R3A | protein phosphatase 2 regulatory subunit B″alpha | 073711 | 174611 | 174579 | 15, 25, 24 |
| 3 | PP2D1 | protein phosphatase 2C like domain containing 1 | 183977 | 144566 | 114166 | 38, 37, 5 |
| 3 | PPP4R2 | protein phosphatase 4 regulatory subunit 2 | 163605 | 172986 | 255423 | 11, 26, 31 |
| 3 | PPM1L | protein phosphatase, Mg2+/Mn2+ dependent 1L | 163590 | 179674 | 169255 | 24, 42, 25 |
| 3 | PPM1M | protein phosphatase, Mg2+/Mn2+ dependent 1M | 164088 | 247596 | 164091 | |
| 3 | PROS1 | protein S (alpha) | 184500 | 044524 | 169379 | 42, 40, 35 |
| 3 | PTPN23 | protein tyrosine phosphatase, non-receptor type 23 | 076201 | 114648 | 114650 | 26, 18, 22 |
| 3 | PTPRG | protein tyrosine phosphatase, receptor type G | 144724 | 189283 | 114405 | 1, 40, 27 |
| 3 | P2RY1 | purinergic receptor P2Y1 | 169860 | 152601 | 181467 | 24, 9, 45 |
| 3 | P2RY12 | purinergic receptor P2Y12 | 169313 | 181631 | 152580 | 8, 17, 42 |
| 3 | P2RY13 | purinergic receptor P2Y13 | 181631 | 138271 | 169313 | 30, 5, 36 |
| 3 | P2RY14 | purinergic receptor P2Y14 | 174944 | 174946 | 138271 | 7, 32, 10 |
| 3 | PXK | PX domain containing serine/threonine kinase like | 168297 | 255154 | 168291 | 1, 8, 22 |
| 3 | PDHB | pyruvate dehydrogenase (lipoamide) beta | 168291 | 168297 | 168301 | 30, 3, 6 |
| 3 | QTRT2 | queuine tRNA-ribosyltransferase accessory subunit 2 | 151576 | 163617 | 151577 | |
| 3 | RABL3 | RAB, member of RAS oncogene family like 3 | 144840 | 113924 | 153767 | 29, 45, 43 |
| 3 | RAB43 | RAB43, member RAS oncogene family | 172780 | 169704 | 261796 | |
| 3 | RAB5A | RAB5A, member RAS oncogene family | 144566 | 163576 | 183977 | 45, 2, 4 |
| 3 | RAB6B | RAB6B, member RAS oncogene family | 154917 | 144867 | 221972 | 29, 8, 26 |
| 3 | RAB7A | RAB7A, member RAS oncogene family | 075785 | 163902 | 177646 | 45, 11, 34 |
| 3 | RBSN | rabenosyn, RAB effector | 131381 | 131368 | 131375 | 9, 14, 33 |
| 3 | RAD18 | RAD18, E3 ubiquitin protein ligase | 070950 | 180914 | 196220 | 45, 7, 34 |
| 3 | RAD54L2 | RAD54-like 2 (S. cerevisiae) | 164080 | 145041 | 164081 | 43, 26, 11 |
| 3 | RAF1 | Raf-1 proto-oncogene, serine/threonine kinase | 132155 | 075975 | 088726 | 39, 24, 42 |
| 3 | RFTN1 | raftlin, lipid raft linker 1 | 131378 | 154814 | 092345 | 41, 21, 22 |
| 3 | RAP2B | RAP2B, member of RAS oncogene family | 181467 | 169860 | 114790 | 41, 4, 6 |
| 3 | RASSF1 | Ras association domain family member 1 | 068028 | 114383 | 004838 | 36, 9, 13 |
| 3 | RHOA | ras homolog family member A | 067560 | 233276 | 145022 | |
| 3 | RASA2 | RAS p21 protein activator 2 | 155903 | 177311 | 280530 | 23, 31, 22 |
| 3 | RTP1 | receptor transporter protein 1 | 175077 | 163923 | 127241 | 38, 8, |
| 3 | RTP2 | receptor transporter protein 2 | 198471 | 157005 | 113916 | 34, |
| 3 | RTP3 | receptor transporter protein 3 | 163825 | 012223 | 163827 | 10, 19, |
| 3 | RTP4 | receptor transporter protein 4 | 136514 | 127241 | 157005 | 4, 35, 6 |
| 3 | RYK | receptor-like tyrosine kinase | 163785 | 174640 | 114019 | 27, 15, 4 |
| 3 | RFC4 | replication factor C subunit 4 | 163918 | 156976 | 181092 | 34, 4, 7 |
| 3 | RETNLB | resistin like beta | 163515 | 198919 | 163519 | 16, |
| 3 | RARB | retinoic acid receptor beta | 077092 | 151090 | 077097 | 24, 43, 11 |
| 3 | RARRES1 | retinoic acid receptor responder 1 | 118849 | 079257 | 118855 | 1, 23, 44 |
| 3 | RBP1 | retinol binding protein 1 | 114115 | 114113 | 163864 | 11, 24, 28 |
| 3 | RBP2 | retinol binding protein 2 | 114113 | 184432 | 114115 | 16, 20, 17 |
| 3 | RFT1 | RFT1 homolog | 163933 | 272305 | 163932 | 9, 6, 21 |
| 3 | ARHGAP31 | Rho GTPase activating protein 31 | 031081 | 121578 | 176142 | 43, 26, 31 |
| 3 | ARHGEF26 | Rho guanine nucleotide exchange factor 26 | 114790 | 181467 | 174953 | 33, 38, 29 |
| 3 | ARHGEF3 | Rho guanine nucleotide exchange factor 3 | 163947 | 163946 | 186451 | 1, 29, 42 |
| 3 | RHO | rhodopsin | 163914 | 163913 | 178804 | 14, 25, 8 |
| 3 | ROPN1 | rhophilin associated tail protein 1 | 065371 | 175455 | 160145 | |
| 3 | ROPN1B | rhophilin associated tail protein 1B | 114547 | 189366 | 114544 | 38, 24, 23 |
| 3 | RPP14 | ribonuclease P/MRP subunit p14 | 163684 | 163686 | 255154 | 30, 13, 29 |
| 3 | RPN1 | ribophorin I | 163902 | 179348 | 075785 | 13, 45, 36 |
| 3 | RPL14 | ribosomal protein L14 | 188846 | 168032 | 177873 | 27, 23, 7 |
| 3 | RPL15 | ribosomal protein L15 | 174748 | 197885 | 174738 | 29, 1, 31 |
| 3 | RPL22L1 | ribosomal protein L22 like 1 | 163584 | 013293 | 163577 | 33, 43, 29 |
| 3 | RPL24 | ribosomal protein L24 | 114391 | 066422 | 182504 | |
| 3 | RPL29 | ribosomal protein L29 | 162244 | 243989 | 164086 | 41, 29, 6 |
| 3 | RPL32 | ribosomal protein L32 | 144713 | 144712 | 144711 | 43, 11, 12 |
| 3 | RPL35A | ribosomal protein L35a | 182899 | 114473 | 185621 | 29, 11, 33 |
| 3 | RPL39L | ribosomal protein L39 like | 163923 | 073849 | 175077 | 23, 10, 5 |
| 3 | RPSA | ribosomal protein SA | 168028 | 144659 | 168314 | 21, 41, 43 |
| 3 | RRP9 | ribosomal RNA processing 9, small subunit (SSU) processome component, | 114767 | 173389 | 041880 | 40, 3, 35 |
| | | homolog (yeast) | | | | |
| 3 | RNF123 | ring finger protein 123 | 164068 | 173531 | 176020 | 43, 38, 31 |
| 3 | RNF13 | ring finger protein 13 | 082996 | 206199 | 070087 | 37, 30, 29 |
| 3 | RNF168 | ring finger protein 168 | 163961 | 163960 | 214097 | 34, 11, 1 |
| 3 | RNF7 | ring finger protein 7 | 114125 | 280530 | 114124 | 30, 26, 37 |
| 3 | RYBP | RING1 and YY1 binding protein | 163602 | 163421 | 144736 | 32, 4, 22 |
| 3 | RBM15B | RNA binding motif protein 15B | 259956 | 145050 | 145041 | 21, 7, 22 |
| 3 | RBM5 | RNA binding motif protein 5 | 003756 | 004534 | 001617 | |
| 3 | RBM6 | RNA binding motif protein 6 | 004534 | 164077 | 003756 | 29, 43, 42 |
| 3 | RBMS3 | RNA binding motif single stranded interacting protein 3 | 144642 | 283563 | 163513 | 26, 1, 42 |
| 3 | POLR2H | RNA polymerase II subunit H | 163882 | 114859 | 090534 | 40, 41, 7 |
| 3 | RPUSD3 | RNA pseudouridylate synthase domain containing 3 | 156990 | 214021 | 187288 | 34, 38, 4 |
| 3 | ROBO1 | roundabout guidance receptor 1 | 169855 | 185008 | 114480 | |
| 3 | ROBO2 | roundabout guidance receptor 2 | 185008 | 227124 | 169855 | 28, 31, 29 |
| 3 | RUBCN | RUN and cysteine rich domain containing beclin 1 interacting protein | 145016 | 161267 | 122068 | 22, 1, 41 |
| 3 | RUVBL1 | RuvB like AAA ATPase 1 | 175792 | 058262 | 132394 | 13, 41, 3 |
| 3 | SACM1L | SAC1 suppressor of actin mutations 1 -like (yeast) | 211456 | 144791 | 163817 | 42, 24, 11 |
| 3 | SLMAP | sarcolemma associated protein | 163681 | 174839 | 136068 | 45, 29, 15 |
| 3 | SATB1 | SATB homeobox 1 | 182568 | 131374 | 183960 | 39, |
| 3 | SCHIP1 | schwannomin interacting protein 1 | 151967 | 283245 | 168811 | 8, 45, 12 |
| 3 | SCHIP1 | schwannomin interacting protein 1 | 283245 | 283154 | 151967 | 8, 45, 12 |
| 3 | SFMBT1 | Scm-like with four mbt domains 1 | 163935 | 213533 | 272305 | 38, 29, 23 |
| 3 | SEC13 | SEC13 homolog, nuclear pore and COPII coat complex component | 157020 | 157017 | 157087 | 30, 9, 12 |
| 3 | SEC22A | SEC22 homolog A, vesicle trafficking protein | 121542 | 065485 | 173175 | 13, 29, 24 |
| 3 | SEC22C | SEC22 homolog C, vesicle trafficking protein | 093183 | 114812 | 008324 | |
| 3 | SEC61A1 | Sec61 translocon alpha 1 subunit | 058262 | 187715 | 175792 | 3, 24, 42 |
| 3 | SEC62 | SEC62 homolog, preprotein translocation factor | 008952 | 187033 | 173890 | 25, 31, 1 |
| 3 | SELENOK | selenoprotein K | 113811 | 113812 | 157445 | |
| 3 | SELENOT | selenoprotein T | 198843 | 144895 | 163645 | 7, 4, 34 |
| 3 | SEMA3B | semaphorin 3B | 012171 | 114353 | 179564 | 32, 26, 2 |
| 3 | SEMA3F | semaphorin 3F | 001617 | 003756 | 114349 | 40, 17, 24 |
| 3 | SEMA3G | semaphorin 3G | 010319 | 010318 | 114854 | 1, 6, 15 |
| 3 | SEMA5B | semaphorin 5B | 082684 | 138463 | 065485 | 12, 8, 17 |
| 3 | SNTN | sentan, cilia apical structure protein | 188817 | 163630 | 163632 | 30, 20, 42 |
| 3 | SPTSSB | serine palmitoyltransferase small subunit B | 196542 | 169251 | 182447 | 4, 43, 13 |
| 3 | SPINK8 | serine peptidase inhibitor, Kazal type 8 (putative) | 229453 | 172113 | 164049 | 10, 5, |
| 3 | SERPINI1 | serpin family I member 1 | 163536 | 114209 | 173905 | 8, 11, 28 |
| 3 | SERPINI2 | serpin family I member 2 | 114204 | 169064 | 174776 | 10, 28, 4 |
| 3 | SETMAR | SET domain and mariner transposase fusion gene | 170364 | 175928 | 150995 | 43, 15, 44 |
| 3 | SETD2 | SET domain containing 2 | 181555 | 160796 | 088727 | 20, 26, 22 |
| 3 | SETD5 | SET domain containing 5 | 168137 | 134077 | 156959 | 31, 40, 22 |
| 3 | STAC | SH3 and cysteine rich domain | 144681 | 172995 | 163673 | 33, 35, 17 |
| 3 | SH3BP5 | SH3 domain binding protein 5 | 131370 | 131375 | 206562 | 2, 29, 1 |
| 3 | SHISA5 | shisa family member 5 | 164054 | 213689 | 114268 | |
| 3 | SHOX2 | short stature homeobox 2 | 168779 | 174899 | 174891 | 15, 24, 35 |
| 3 | SHQ1 | SHQ1, H/ACA ribonucleoprotein assembly factor | 144736 | 163602 | 172986 | |
| 3 | SGO1 | shugoshin 1 | 129810 | 114166 | 151789 | |
| 3 | SIAH2 | siah E3 ubiquitin protein ligase 2 | 181788 | 163645 | 280141 | |
| 3 | SIDT1 | SID1 transmembrane family member 1 | 072858 | 163611 | 176542 | 39, 42, 36 |
| 3 | SPCS1 | signal peptidase complex subunit 1 | 114902 | 016864 | 114904 | 29, 7, 30 |
| 3 | SSR3 | signal sequence receptor subunit 3 | 114850 | 169282 | 163659 | 45, 42, 40 |
| 3 | SMCO1 | single-pass membrane protein with coiled-coil domains 1 | 214097 | 163961 | 185798 | 25, 15, 10 |
| 3 | SKIL | SKI like proto-oncogene | 136603 | 163558 | 013297 | |
| 3 | SLITRK3 | SLIT and NTRK like family member 3 | 121871 | 090402 | 114200 | 8, 44, |
| 3 | SRGAP3 | SLIT-ROBO Rho GTPase activating protein 3 | 196220 | 070950 | 134077 | 43, 26, 8 |
| 3 | SMIM4 | small integral membrane protein 4 | 168273 | 168268 | 163939 | 15, 4, 33 |
| 3 | SNRK | SNF related kinase | 163788 | 144647 | 160746 | 29, 45, 26 |
| 3 | SCN10A | sodium voltage-gated channel alpha subunit 10 | 185313 | 183873 | 168356 | 25, |
| 3 | SCN11A | sodium voltage-gated channel alpha subunit 11 | 168356 | 185313 | 114742 | 32, 38, |
| 3 | SCN5A | sodium voltage-gated channel alpha subunit 5 | 183873 | 157036 | 185313 | 27, 15, 14 |
| 3 | SLC12A8 | solute carrier family 12 member 8 | 221955 | 173706 | 163848 | 13, 33, 18 |
| 3 | SLC15A2 | solute carrier family 15 member 2 | 163406 | 145088 | 145103 | 42, 24, 33 |
| 3 | SLC2A2 | solute carrier family 2 member 2 | 163581 | 163577 | 154310 | 19, 17, 36 |
| 3 | SLC22A13 | solute carrier family 22 member 13 | 172940 | 172939 | 144671 | 17, |
| 3 | SLC22A14 | solute carrier family 22 member 14 | 144671 | 172940 | 093217 | 38, 37, 17 |
| 3 | SLC25A20 | solute carrier family 25 member 20 | 178537 | 114302 | 221883 | 29, 5, 15 |
| 3 | SLC25A26 | solute carrier family 25 member 26 | 144741 | 151276 | 144749 | 3, 32, 4 |
| 3 | SLC25A36 | solute carrier family 25 member 36 | 114120 | 155890 | 175093 | 26, 22, 17 |
| 3 | SLC25A38 | solute carrier family 25 member 38 | 144659 | 179934 | 168028 | 39, 3, 2 |
| 3 | SLC26A6 | solute carrier family 26 member 6 | 225697 | 183396 | 008300 | 27, 37, 5 |
| 3 | SLC33A1 | solute carrier family 33 member 1 | 169359 | 174928 | 163655 | 42, 39, 24 |
| 3 | SLC35A5 | solute carrier family 35 member A5 | 138459 | 144848 | 091986 | 39, 45, 24 |
| 3 | SLC35G2 | solute carrier family 35 member G2 | 168917 | 118007 | 158092 | |
| 3 | SLC38A3 | solute carrier family 38 member 3 | 188338 | 114349 | 114353 | 14, 19, 26 |
| 3 | SLC4A7 | solute carrier family 4 member 7 | 033867 | 163491 | 163508 | 1, 25, 26 |
| 3 | SLC41A3 | solute carrier family 41 member 3 | 114544 | 114547 | 144908 | 8, 2, 38 |
| 3 | SLC51A | solute carrier family 51 alpha subunit | 163959 | 163958 | 161217 | |
| 3 | SLC6A1 | solute carrier family 6 member 1 | 157103 | 132164 | 196639 | 29, 8, 4 |
| 3 | SLC6A11 | solute carrier family 6 member 11 | 132164 | 157087 | 157103 | 18, 25, 19 |
| 3 | SLC6A20 | solute carrier family 6 member 20 | 163817 | 211456 | 163818 | 26, 16, 18 |
| 3 | SLC6A6 | solute carrier family 6 member 6 | 131389 | 170860 | 144596 | 5, 24, 4 |
| 3 | SLC7A14 | solute carrier family 7 member 14 | 013293 | 013297 | 163584 | 31, 45, 8 |
| 3 | SLC9A9 | solute carrier family 9 member A9 | 181804 | 175040 | 181744 | 42, 39, 26 |
| 3 | SLC9C1 | solute carrier family 9 member C1 | 172139 | 174500 | 091972 | 38, 25, |
| 3 | SLC02A1 | solute carrier organic anion transporter family member 2A1 | 174640 | 221972 | 163785 | 28, 42, 17 |
| 3 | SST | somatostatin | 157005 | 136514 | 198471 | 28, 37, 15 |
| 3 | SNX4 | sorting nexin 4 | 114520 | 163848 | 144909 | 45, 43, 42 |
| 3 | SPATA12 | spermatogenesis associated 12 | 186451 | 163947 | 144730 | 38, 12, 32 |
| 3 | SPATA16 | spermatogenesis associated 16 | 144962 | 114346 | 169760 | 38, 10, 8 |
| 3 | SPICE1 | spindle and centriole associated protein 1 | 163611 | 283241 | 072858 | 39, 42, 37 |
| 3 | SPSB4 | splA/ryanodine receptor domain and SOCS box containing 4 | 175093 | 114120 | 155893 | 15, 27, 25 |
| 3 | SOAP | SREBF chaperone | 114650 | 076201 | 163832 | 38, 39, 42 |
| 3 | SRPRB | SRP receptor beta subunit | 144867 | 091513 | 154917 | 3, 4, 28 |
| 3 | SOX14 | SRY-box 14 | 168875 | 174564 | 066405 | 32, 17, |
| 3 | SOX2 | SRY-box2 | 181449 | 205981 | 058063 | 13, 25, 12 |
| 3 | SS18L2 | SS18 like 2 | 008324 | 093183 | 114857 | 29, 43, 7 |
| 3 | SSUH2 | ssu-2 homolog (C. elegans) | 125046 | 071282 | 182533 | |
| 3 | ST3GAL6 | ST3 beta-galactoside alpha-2,3-sialyltransferase 6 | 064225 | 154165 | 057019 | 43, 35, 1 |
| 3 | ST6GAL1 | ST6 beta-galactoside alpha-2,6-sialyltransferase 1 | 073849 | 181092 | 163923 | 22, 42, 40 |
| 3 | STAB1 | stabilin 1 | 010327 | 010322 | 168268 | 1, 7, 26 |
| 3 | SAMD7 | sterile alpha motif domain containing 7 | 187033 | 114248 | 008952 | 25, 14, |
| 3 | SERP1 | stress associated endoplasmic reticulum protein 1 | 120742 | 196428 | 144895 | 29, 41, 10 |
| 3 | STAG1 | stromal antigen 1 | 118007 | 114054 | 168917 | 1, 25, 39 |
| 3 | SMC4 | structural maintenance of chromosomes 4 | 113810 | 068885 | 213186 | 22, 39, 4 |
| 3 | STT3B | STT3B, catalytic subunit of the oligosaccharyltransferase complex | 163527 | 144644 | 144645 | 29, 32, 3 |
| 3 | SUCNR1 | succinate receptor 1 | 198829 | 114771 | 152601 | 17, 5, 19 |
| 3 | SUCLG2 | succinate-CoA ligase GDP-forming beta subunit | 172340 | 163376 | 183662 | 7, 9, 19 |
| 3 | SI | sucrase-isomaltase | 090402 | 182447 | 121871 | 10, 16, 25 |
| 3 | SUMF1 | sulfatase modifying factor 1 | 144455 | 113851 | 175928 | |
| 3 | SENP5 | SUMO1/sentrin specific peptidase 5 | 119231 | 180370 | 114503 | 24, 16, 35 |
| 3 | SENP7 | SUMO1/sentrin specific peptidase 7 | 138468 | 081148 | 174173 | 13, 26, 31 |
| 3 | SENP2 | SUMO1/sentrin/SMT3 specific peptidase 2 | 163904 | 163898 | 073792 | 24, 2, 38 |
| 3 | SUSD5 | sushi domain containing 5 | 173705 | 170275 | 153558 | 45, 42, 7 |
| 3 | SMARCC1 | SWI/SNF related, matrix associated, actin dependent regulator of chromatin | 173473 | 114646 | 132153 | 4, 12, 22 |
| | | subfamily c member 1 | | | | |
| 3 | SYN2 | synapsin II | 157152 | 144559 | 157150 | 26, 29, 8 |
| 3 | SYNPR | synaptoporin | 163630 | 163618 | 188817 | 8, 17, 14 |
| 3 | STX19 | syntaxin 19 | 178750 | 169379 | 178700 | 35, |
| 3 | STXBP5L | syntaxin binding protein 5 like | 145087 | 153767 | 051341 | 8, 40, 20 |
| 3 | TRAT1 | T cell receptor associated transmembrane adaptor 1 | 163519 | 163515 | 138472 | 39, 30, 5 |
| 3 | TAMM41 | TAM41 mitochondrial translocator assembly and maintenance homolog | 144559 | 144560 | 157152 | 43, 29, 3 |
| 3 | TMF1 | TATA element modulatory factor 1 | 144747 | 163378 | 144744 | 22, 45, 38 |
| 3 | TATDN2 | TatD DNase domain containing 2 | 157014 | 134070 | 272410 | 43, 22, 21 |
| 3 | TBC1D23 | TBC1 domain family member 23 | 036054 | 168386 | 114021 | 42, 43, 24 |
| 3 | TBC1D5 | TBC1 domain family member 5 | 131374 | 154822 | 182568 | 29, 22, 41 |
| 3 | TBCCD1 | TBCC domain containing 1 | 113838 | 213139 | 090520 | 38, 24, 13 |
| 3 | TIPARP | TCDD inducible poly(ADP-ribose) polymerase | 163659 | 114850 | 197980 | 29, 9, 42 |
| 3 | TCAIM | T-cell activation inhibitor, mitochondrial | 179152 | 173769 | 225873 | 22, 7, 4 |
| 3 | TIGIT | T-cell immunoreceptor with Ig and ITIM domains | 181847 | 174255 | 181722 | 36, 24, 39 |
| 3 | TCTA | T-cell leukemia translocation altered | 145022 | 067560 | 145020 | 2, 23, 15 |
| 3 | TCTEX1D2 | Tctex1 domain containing 2 | 213123 | 272741 | 273331 | 28, 15, 14 |
| 3 | TDGF1 | teratocarcinoma-derived growth factor 1 | 241186 | 163827 | 283473 | 12, 38, 36 |
| 3 | TOPAZ1 | testis and ovary specific PAZ domain containing 1 | 173769 | 011198 | 179152 | 38, |
| 3 | TEX264 | testis expressed 264 | 164081 | 164080 | 164082 | 18, 21, 6 |
| 3 | TRANK1 | tetratricopeptide repeat and ankyrin repeat containing 1 | 168016 | 163673 | 178567 | 21, 41, 22 |
| 3 | TTC14 | tetratricopeptide repeat domain 14 | 163728 | 114757 | 145075 | |
| 3 | TTC21A | tetratricopeptide repeat domain 21A | 168026 | 114745 | 144655 | 38, 42, 40 |
| 3 | TXNRD3 | thioredoxin reductase 3 | 197763 | 206483 | 159685 | 38, 26, 33 |
| 3 | TXNRD3NB | thioredoxin reductase 3 neighbor | 206483 | 283374 | 197763 | |
| 3 | TXNRD3NB | thioredoxin reductase 3 neighbor | 283374 | 180697 | 206483 | |
| 3 | THOC7 | THO complex 7 | 163634 | 163632 | 163635 | 4, 29, 18 |
| 3 | TREX1 | three prime repair exonuclease 1 | 213689 | 164053 | 164054 | 9, |
| 3 | THPO | thrombopoietin | 090534 | 163882 | 090539 | |
| 3 | THUMPD3 | THUMP domain containing 3 | 134077 | 196220 | 168137 | 18, 11, 38 |
| 3 | THRB | thyroid hormone receptor beta | 151090 | 174738 | 077092 | 4, 23, 1 |
| 3 | TRH | thyrotropin releasing hormone | 170893 | 172765 | 251287 | 27, 14, 20 |
| 3 | TIMP4 | TIMP metallopeptidase inhibitor 4 | 157150 | 157152 | 132170 | 1, 31, 3 |
| 3 | TM4SF19- | TM4SF19-TCTEX1D2 readthrough (NMD candidate) | 273331 | 213123 | 145107 | |
| | TCTEX1D2 | | | | | |
| 3 | TMEM110- | TMEM110-MUSTN1 readthrough | 248592 | 272573 | 213533 | |
| | MUSTN1 | | | | | |
| 3 | TLR9 | toll like receptor 9 | 239732 | 023330 | 173366 | 41, 22, 42 |
| 3 | TOP2B | topoisomerase (DNA) II beta | 077097 | 077092 | 151092 | |
| 3 | TOPBP1 | topoisomerase (DNA) II binding protein 1 | 163781 | 091527 | 091513 | |
| 3 | TRAIP | TRAF interacting protein | 183763 | 182179 | 164076 | 43, 21, 5 |
| 3 | TNIK | TRAF2 and NCK interacting kinase | 154310 | 163581 | 075651 | 3, 40, 38 |
| 3 | TRAK1 | trafficking kinesin protein 1 | 182606 | 168038 | 187094 | 22, 3, 18 |
| 3 | TFDP2 | transcription factor Dp-2 | 114126 | 069849 | 175066 | |
| 3 | TADA3 | transcriptional adaptor 3 | 171148 | 134072 | 241553 | 9, 21, 27 |
| 3 | TBL1XR1 | transducin beta like 1 X-linked receptor 1 | 177565 | 177694 | 197584 | 11, 4, 34 |
| 3 | TF | transferrin | 091513 | 163781 | 144867 | 19, 14, 8 |
| 3 | TFRC | transferrin receptor | 072274 | 061938 | 163958 | 24, 13, 32 |
| 3 | TRA2B | transformer 2 beta homolog (Drosophila) | 136527 | 073792 | 244405 | 39, 11, 4 |
| 3 | TGFBR2 | transforming growth factor beta receptor 2 | 163513 | 144642 | 144644 | 11, 1, 10 |
| 3 | TAGLN3 | transgelin 3 | 144834 | 144827 | 176040 | 8, 14, 26 |
| 3 | TGM4 | transglutaminase 4 | 163810 | 169964 | 163812 | 33, 25, 34 |
| 3 | TRPC1 | transient receptor potential cation channel subfamily C member 1 | 144935 | 120756 | 163710 | 26, 12, 17 |
| 3 | TKT | transketolase | 163931 | 163932 | 272886 | 5, 9, 39 |
| 3 | TMA7 | translation machinery associated 7 homolog | 232112 | 164051 | 164053 | 7, 43, 2 |
| 3 | TIMMDC1 | translocase of inner mitochondrial membrane domain containing 1 | 113845 | 163389 | 121594 | 6, 26, 17 |
| 3 | TOMM70 | translocase of outer mitochondrial membrane 70 | 154174 | 114021 | 206535 | 13, 31, 45 |
| 3 | TM4SF1 | transmembrane 4 L six family member 1 | 169908 | 163762 | 169903 | 1, 45, 3 |
| 3 | TM4SF18 | transmembrane 4 L six family member 18 | 163762 | 047457 | 169908 | 43, 17, 3 |
| 3 | TM4SF19 | transmembrane 4 L six family member 19 | 145107 | 273331 | 163960 | 13, 6, 35 |
| 3 | TM4SF4 | transmembrane 4 L six family member 4 | 169903 | 169908 | 018408 | 28, 19, 3 |
| 3 | TMCC1 | transmembrane and coiled-coil domain family 1 | 172765 | 004399 | 170893 | 31, 4, 23 |
| 3 | TMIE | transmembrane inner ear | 181585 | 178038 | 206549 | 14, 2, 25 |
| 3 | TMPRSS7 | transmembrane protease, serine 7 | 176040 | 144834 | 114529 | 25, 38, |
| 3 | TMEM108 | transmembrane protein 108 | 144868 | 113971 | 170819 | 38, 20, 14 |
| 3 | TMEM110 | transmembrane protein 110 | 213533 | 248592 | 163935 | |
| 3 | TMEM115 | transmembrane protein 115 | 126062 | 272104 | 007402 | 27, 9, 33 |
| 3 | TMEM158 | transmembrane protein 158 (gene/pseudogene) | 249992 | 163814 | 011376 | 4, 6, 34 |
| 3 | TMEM207 | transmembrane protein 207 | 198398 | 113946 | 196083 | 17, 10, |
| 3 | TMEM212 | transmembrane protein 212 | 186329 | 075651 | 075420 | 30, 42, 20 |
| 3 | TMEM39A | transmembrane protein 39A | 176142 | 031081 | 163389 | 16, 38, |
| 3 | TMEM40 | transmembrane protein 40 | 088726 | 132155 | 144712 | 32, 22, 30 |
| 3 | TMEM41A | transmembrane protein 41A | 163900 | 073803 | 163898 | 2, 11, 13 |
| 3 | TMEM42 | transmembrane protein 42 | 169964 | 163808 | 163810 | 11, 26, 31 |
| 3 | TMEM43 | transmembrane protein 43 | 170876 | 163528 | 268279 | 7, 45, 10 |
| 3 | TMEM44 | transmembrane protein 44 | 145014 | 133657 | 041802 | 6, 14, 2 |
| 3 | TMEM45A | transmembrane protein 45A | 181458 | 206535 | 144820 | 10, 30, 35 |
| 3 | TMEM89 | transmembrane protein 89 | 183396 | 010256 | 225697 | 38, 8, |
| 3 | TPRA1 | transmembrane protein adipocyte associated 1 | 163870 | 114554 | 073111 | 26, 9, 21 |
| 3 | TMPPE | transmembrane protein with metallophosphoesterase domain | 188167 | 170266 | 170275 | 7, 13, 36 |
| 3 | TRIM42 | tripartite motif containing 42 | 155890 | 158258 | 114120 | 38, 10, |
| 3 | TRIM59 | tripartite motif containing 59 | 213186 | 113810 | 186432 | 4, 22, 39 |
| 3 | TRIM71 | tripartite motif containing 71 | 206557 | 182973 | 183813 | 12, 27, 38 |
| 3 | TFG | TRK-fused gene | 114354 | 144820 | 154175 | 10, 29, 43 |
| 3 | TRMT10C | tRNA methyltransferase 10C, mitochondrial RNase P subunit | 174173 | 138468 | 081154 | 1, 22, 34 |
| 3 | TRNT1 | tRNA nucleotidyl transferase 1 | 072756 | 091181 | 281584 | 13, 4, 29 |
| 3 | TSEN2 | tRNA splicing endonuclease subunit 2 | 154743 | 132170 | 225526 | 28, 21, 25 |
| 3 | TNNC1 | troponin C1, slow skeletal and cardiac type | 114854 | 010319 | 010322 | 15, 25, 18 |
| 3 | TSC22D2 | TSC22 domain family member 2 | 196428 | 070087 | 120742 | |
| 3 | TTLL3 | tubulin tyrosine ligase like 3 | 214021 | 250151 | 156990 | 33, 29, 26 |
| 3 | TNFSF10 | tumor necrosis factor superfamily member 10 | 121858 | 121853 | 144959 | 13, 30, 2 |
| 3 | TP63 | tumor protein p63 | 073282 | 188001 | 090530 | 24, 4, 15 |
| 3 | TPRG1 | tumor protein p63 regulated 1 | 188001 | 145012 | 073282 | 10, 23, 24 |
| 3 | TUSC2 | tumor suppressor candidate 2 | 114383 | 068001 | 068028 | 32, 27, 14 |
| 3 | TWF2 | twinfilin actin binding protein 2 | 247596 | 173366 | 164088 | 41, 21, 34 |
| 3 | TNK2 | tyrosine kinase non receptor 2 | 061938 | 145113 | 072274 | 26, 22, 13 |
| 3 | U2SURP | U2 snRNP associated SURP domain containing | 163714 | 188582 | 175040 | 10, 1, 26 |
| 3 | UQCRC1 | ubiquinol-cytochrome c reductase core protein I | 010256 | 114270 | 183396 | 27, 35, 25 |
| 3 | UBE2E1 | ubiquitin conjugating enzyme E2 E1 | 170142 | 182247 | 197885 | 29, 43, 11 |
| 3 | UBE2E2 | ubiquitin conjugating enzyme E2 E2 | 182247 | 151789 | 170142 | 10, 43, 26 |
| 3 | UBA3 | ubiquitin like modifier activating enzyme 3 | 144744 | 144747 | 144746 | 45, 9, 30 |
| 3 | UBA5 | ubiquitin like modifier activating enzyme 5 | 081307 | 129048 | 113971 | 42, 45, 24 |
| 3 | UBA7 | ubiquitin like modifier activating enzyme 7 | 182179 | 185614 | 183763 | 7, 22, 26 |
| 3 | USP13 | ubiquitin specific peptidase 13 (isopeptidase T-3) | 058056 | 136521 | 114757 | 9, 18, 25 |
| 3 | USP19 | ubiquitin specific peptidase 19 | 172046 | 172053 | 172037 | 21, 26, 25 |
| 3 | USP4 | ubiquitin specific peptidase 4 | 114316 | 188315 | 233276 | |
| 3 | UBXN7 | UBX domain protein 7 | 163960 | 145107 | 163961 | 18, 31, 43 |
| 3 | B3GNT5 | UDP-GlcNAc: betaGal beta-1,3-N-acetylglucosaminyltransferase 5 | 176597 | 053524 | 172578 | |
| 3 | ULK4 | unc-51 like kinase 4 | 168038 | 168036 | 182606 | 38, 42, 7 |
| 3 | FLJ22763 | Uncharacterized LOC401081 | 241224 | 114487 | 163530 | 37, 17, 10 |
| 3 | UBP1 | upstream binding protein 1 (LBP-1a) | 153560 | 153558 | 163539 | 34, 26, 39 |
| 3 | USF3 | upstream transcription factor family member 3 | 176542 | 072858 | 121579 | 22, 26, 28 |
| 3 | UMPS | uridine monophosphate synthetase | 114491 | 160145 | 082781 | 30, 21, 9 |
| 3 | UROC1 | urocanate hydratase 1 | 159650 | 070476 | 180767 | 19, 23, |
| 3 | UCN2 | urocertin 2 | 145040 | 114268 | 114270 | 35, 44, 38 |
| 3 | UPK1B | uroplakin 1B | 114638 | 251012 | 121578 | 30, 42, 13 |
| 3 | UTS2B | urotensin 2B | 188958 | 188729 | 152492 | 38, 8, 33 |
| 3 | VIPR1 | vasoactive intestinal peptide receptor 1 | 114812 | 157093 | 093183 | |
| 3 | VEPH1 | ventricular zone expressed PH domain containing 1 | 197415 | 163660 | 163661 | 43, 10, 45 |
| 3 | VGLL3 | vestigial like family member 3 | 206538 | 175161 | 083937 | 32, 1, 26 |
| 3 | VGLL4 | vestigial like family member 4 | 144560 | 197548 | 144559 | 42, 24, 44 |
| 3 | VILL | villin like | 136059 | 144677 | 187091 | 39, 16, 28 |
| 3 | VHL | von Hippel-Lindau tumor suppressor | 134086 | 254999 | 134070 | 34, 43, 11 |
| 3 | VWA5B2 | vpn Willebrand factor A domain containing 5B2 | 145198 | 161204 | 214160 | 2, 28, 8 |
| 3 | VPS8 | VPS8, CORVET complex subunit | 156931 | 177383 | 187068 | |
| 3 | WDR48 | WD repeat domain 48 | 114742 | 168356 | 114745 | 42, 38, 39 |
| 3 | WDR49 | WD repeat domain 49 | 174776 | 114204 | 114209 | 42, 7, 38 |
| 3 | WDR53 | WD repeat domain 53 | 185798 | 214097 | 174013 | 9, 15, 38 |
| 3 | WDR5B | WD repeat domain 5B | 196981 | 114023 | 114030 | 39, 31, 32 |
| 3 | WDR6 | WD repeat domain 6 | 178252 | 178467 | 178149 | 43, 42, 39 |
| 3 | WDR82 | WD repeat domain 82 | 164091 | 164088 | 168237 | 42, 40, 24 |
| 3 | WNT5A | Wnt family member 5A | 114251 | 144771 | 187672 | 26, 4, 6 |
| 3 | WNT7A | Wnt family member 7A | 154764 | 163520 | 163528 | 36, 32, 44 |
| 3 | WWTR1 | WW domain containing transcription regulator 1 | 018408 | 169903 | 114744 | 11, 43, 24 |
| 3 | XCR1 | X-C motif chemokine receptor 1 | 173578 | 172215 | 183625 | 32, |
| 3 | XIRP1 | xin actin binding repeat containing 1 | 168334 | 144655 | 168329 | 18, 15, 25 |
| 3 | XPC | XPC complex subunit, DNA damage recog nition and repair factor | 154767 | 268279 | 170860 | 42, 1, 14 |
| 3 | XXYLT1 | xyloside xylosyltransferase 1 | 173950 | 185112 | 114331 | 45, 24, 27 |
| 3 | XYLB | xylulokinase | 093217 | 144671 | 114739 | 9, 23, 35 |
| 3 | YEATS2 | YEATS domain containing 2 | 163872 | 114796 | 180834 | 41, 13, 21 |
| 3 | ZIC1 | Zic family member 1 | 152977 | 174963 | 144891 | |
| 3 | ZIC4 | Zic family member 4 | 174963 | 231213 | 152977 | 11, 8, 10 |
| 3 | ZBTB11 | zinc finger and BTB domain containing 11 | 066422 | 081154 | 114391 | 34, 26, 2 |
| 3 | ZBTB20 | zinc finger and BTB domain containing 20 | 181722 | 181847 | 279131 | 42, 11, 22 |
| 3 | ZBTB38 | zinc finger and BTB domain containing 38 | 177311 | 155893 | 155903 | |
| 3 | ZBTB47 | zinc finger and BTB domain containing 47 | 114853 | 114857 | 157119 | 15, 40, 26 |
| 3 | ZBBX | zinc finger B-box domain containing | 169064 | 114200 | 114204 | 38, 31, 20 |
| 3 | ZBED2 | zinc finger BED-type containing 2 | 177494 | 153283 | 240891 | 22, 40, 18 |
| 3 | ZCWPW2 | zinc finger CW-type and PWWP domain containing 2 | 206559 | 163512 | 283563 | 34, 10, 28 |
| 3 | ZDHHC19 | zinc finger DHHC-type containing 19 | 163958 | 072274 | 163959 | 38, 12, 32 |
| 3 | ZDHHC23 | zinc finger DHHC-type containing 23 | 184307 | 178075 | 163617 | 24, 40, 13 |
| 3 | ZDHHC3 | zinc finger DHHC-type containing 3 | 163812 | 163810 | 075914 | 3, 9, 33 |
| 3 | ZMAT3 | zinc finger matrin-type 3 | 172667 | 275163 | 121879 | 26, 35, 45 |
| 3 | ZMYND10 | zinc finger MYND-type containing 10 | 004838 | 068028 | 114388 | 38, 9, 20 |
| 3 | ZNF148 | zinc finger protein 148 | 163848 | 221955 | 114520 | 31, 24, 30 |
| 3 | ZNF197 | zinc finger protein 197 | 186448 | 144792 | 169981 | 45, 40, 13 |
| 3 | ZNF35 | zinc finger protein 35 | 169981 | 186448 | 196653 | |
| 3 | ZNF385D | zinc finger protein 385D | 151789 | 129810 | 182247 | |
| 3 | ZNF445 | zinc finger protein 445 | 185219 | 225873 | 178917 | 12, 14, 18 |
| 3 | ZNF501 | zinc finger protein 501 | 186446 | 196653 | 163807 | 30, 2, 8 |
| 3 | ZNF502 | zinc finger protein 502 | 196653 | 169981 | 186446 | 31, 2, 3 |
| 3 | ZNF589 | zinc finger protein 589 | 164048 | 164047 | 172113 | 1, 43, 40 |
| 3 | ZNF619 | zinc finger protein 619 | 177873 | 188846 | 177842 | 40, 42, 45 |
| 3 | ZNF620 | zinc finger protein 620 | 177842 | 177873 | 172888 | 29, 39, 23 |
| 3 | ZNF621 | zinc finger protein 621 | 172888 | 177842 | 168036 | 37, 33, 15 |
| 3 | ZNF639 | zinc finger protein 639 | 121864 | 171121 | 171109 | 29, 31, 27 |
| 3 | ZNF654 | zinc finger protein 654 | 175105 | 163320 | 179021 | 22, 11, 45 |
| 3 | ZNF660 | zinc finger protein 660 | 144792 | 196345 | 186448 | 40, 45, 27 |
| 3 | ZNF662 | zinc finger protein 662 | 182983 | 180432 | 144649 | 33, 18, 20 |
| 3 | ZNF717 | zinc finger protein 717 | 227124 | 172969 | 185008 | 14, 38, 8 |
| 3 | ZNF80 | zinc finger protein 80 | 174255 | 151577 | 181847 | 45, 22, 20 |
| 3 | ZNF852 | zinc finger protein 852 | 178917 | 185219 | 196345 | 34, 30, 7 |
| 3 | ZNF860 | zinc finger protein 860 | 197385 | 144645 | 152642 | 22, 28, 24 |
| 3 | ZKSCAN7 | zinc finger with KRAB and SCAN domains 7 | 196345 | 178917 | 144792 | 1, 2, 38 |
| 3 | ZPLD1 | zona pellucida like domain containing 1 | 170044 | 144802 | 170017 | 14, 38, |
| 3 | ZXDC | ZXD family zinc finger C | 070476 | 163885 | 159650 | |

Table 3 provides the gene information for the genes found on Chromosomes 4-6.

TABLE 3
Genes on Chromosomes 4-6

| | | | | Up | Down | Tissue |
|---|---|---|---|---|---|---|
| Chr. | Gene Symbol | Gene Name | ENSG | ENSG | ENSG | Code |
| 4 | BDH2 | 3-hydroxybutyrate dehydrogenase 2 | 164039 | 164038 | 138778 | 29, 44, 4 |
| 4 | PAPSS1 | 3′-phosphoadenosine 5′-phosphosulfate synthase 1 | 138801 | 155011 | 164023 | 45, 30, 42 |
| 4 | ABHD18 | abhydrolase domain containing 18 | 164074 | 164073 | 138709 | |
| 4 | ASIC5 | acid sensing ion channel subunit family member 5 | 256394 | 061918 | 151790 | |
| 4 | ABLIM2 | actin binding LIM protein family member 2 | 163995 | 196526 | 125089 | 28, 24, 26 |
| 4 | AFAP1 | actin filament associated protein 1 | 196526 | 178597 | 163995 | 45, 12, 6 |
| 4 | ACOX3 | acyl-CoA oxidase 3, pristanoyl | 087008 | 170801 | 155275 | 45, 18, 36 |
| 4 | ACSL1 | acyl-CoA synthetase long-chain family member 1 | 151726 | 151725 | 187821 | 42, 1, 24 |
| 4 | ADAM29 | ADAM metallopeptidase domain 29 | 168594 | 145451 | 150625 | |
| 4 | ADAMTS3 | ADAM metallopeptidase with thrombospondin type 1 motif 3 | 156140 | 056291 | 163626 | 2, 23, 32 |
| 4 | AP1AR | adaptor related protein complex 1 associated regulatory protein | 138660 | 174749 | 145365 | 3, 44, 7 |
| 4 | ADD1 | adducin 1 | 087274 | 087266 | 109736 | 26, 14, 27 |
| 4 | ADAD1 | adenosine deaminase domain containing 1 | 164113 | 138688 | 109471 | 38, 10, 33 |
| 4 | ADGRA3 | adhesion G protein-coupled receptor A3 | 152990 | 185774 | 249948 | 11, 24, 9 |
| 4 | ADGRL3 | adhesion G protein-coupled receptor L3 | 150471 | 163453 | 205678 | |
| 4 | ARFIP1 | ADP ribosylation factor interacting protein 1 | 164144 | 169989 | 137460 | |
| 4 | ARL9 | ADP ribosylation factor like GTPase 9 | 196503 | 174780 | 249693 | 3, 38, 15 |
| 4 | ART3 | ADP-ribosyltransferase 3 | 156219 | 138755 | 169245 | 25, 38, 19 |
| 4 | ADRA2C | adrenoceptor alpha 2C | 184160 | 280230 | 163982 | 32, 42, 23 |
| 4 | AFF1 | AF4/FMR2 family member 1 | 172493 | 163633 | 145332 | 11, 32, 17 |
| 4 | AFM | afamin | 079557 | 081051 | 169435 | 19, 17, 23 |
| 4 | ALB | albumin | 163631 | 132466 | 081051 | 19, 15, 23 |
| 4 | ADH1A | alcohol dehydrogenase 1A (class I), alpha polypeptide | 187758 | 172955 | 196616 | 19, 10, 22 |
| 4 | ADH1B | alcohol dehydrogenase 1B (class I), beta polypeptide | 196616 | 187758 | 248144 | 1, 42, 19 |
| 4 | ADH1C | alcohol dehydrogenase 1C (class I), gamma polypeptide | 248144 | 196616 | 196344 | 16, 19, 30 |
| 4 | ADH4 | alcohol dehydrogenase 4 (class II), pi polypeptide | 198099 | 197894 | 172955 | 19, 23, 5 |
| 4 | ADH5 | alcohol dehydrogenase 5 (class III), chi polypeptide | 197894 | 164024 | 198099 | 42, 12, 44 |
| 4 | ADH6 | alcohol dehydrogenase 6 (class V) | 172955 | 198099 | 187758 | 19, 17, 16 |
| 4 | ADH7 | alcohol dehydrogenase 7 (class IV), mu or sigma polypeptide | 196344 | 248144 | 138813 | 13, 42, 24 |
| 4 | AFP | alpha fetoprotein | 081051 | 163631 | 079557 | 15, 19, 36 |
| 4 | ALPK1 | alpha kinase 1 | 073331 | 145365 | 178403 | 29, 22, 36 |
| 4 | AMBN | ameloblastin | 178522 | 187689 | 132464 | 10, 17, 8 |
| 4 | AMTN | amelotin | 187689 | 171195 | 178522 | 8, |
| 4 | AIMP1 | aminoacyl tRNA synthetase complex interacting multifunctional | 164022 | 145348 | 250298 | 13, 30, 6 |
| | | protein 1 | | | | |
| 4 | AADAT | aminoadipate aminotransferase | 109576 | 198948 | 174473 | 11, 10, 12 |
| 4 | AASDH | aminoadipate-semialdehyde dehydrogenase | 157426 | 109265 | 128059 | 42, 3, 26 |
| 4 | AREG | amphiregulin | 109321 | 124882 | 174808 | 13, 37, 19 |
| 4 | APBB2 | amyloid beta precursor protein binding family B member 2 | 163697 | 179299 | 154277 | 43, 1, 42 |
| 4 | ANAPC10 | anaphase promoting complex subunit 10 | 164162 | 164161 | 164163 | 10, 31, 7 |
| 4 | ANAPC4 | anaphase promoting complex subunit 4 | 053900 | 168228 | 157765 | 1, 42, 15 |
| 4 | ANK2 | ankyrin 2 | 145362 | 174720 | 145349 | 26, 8, 25 |
| 4 | ASB5 | ankyrin repeat and SOCS box containing 5 | 164122 | 150628 | 129128 | 26, 25, 15 |
| 4 | ANKRD17 | ankyrin repeat domain 17 | 132466 | 163626 | 163631 | 18, 22, 1 |
| 4 | ANKRD37 | ankyrin repeat domain 37 | 186352 | 109771 | 109775 | 11, 6, 14 |
| 4 | ANKRD50 | ankyrin repeat domain 50 | 151458 | 164056 | 196159 | 26, 11, 45 |
| 4 | ANXA10 | annexin A10 | 109511 | 196104 | 137628 | 4, 37, 3 |
| 4 | ANXA3 | annexin A3 | 138772 | 138759 | 138756 | 7, 1, 34 |
| 4 | ANXA5 | annexin A5 | 164111 | 186867 | 164112 | 30, 35, 32 |
| 4 | ANTXR2 | anthrax toxin receptor 2 | 163297 | 196475 | 152784 | 1, 44, 45 |
| 4 | APELA | apelin receptor early endogenous ligand | 248329 | 248771 | 183439 | 6, 12, 33 |
| 4 | ARAP2 | ArfGAP with RhoGAP domain, ankyrin repeat and PH domain 2 | 047365 | 169851 | 197057 | |
| 4 | ARSJ | arylsulfatase family member J | 180801 | 145349 | 174607 | |
| 4 | AGA | aspartylglucosaminidase | 038002 | 109674 | 218336 | 9, 1, 13 |
| 4 | ATOH1 | atonal bHLH transcription factor 1 | 172238 | 152208 | 163104 | |
| 4 | ABCE1 | ATP binding cassette subfamily E member 1 | 164163 | 164162 | 164164 | |
| 4 | ABCG2 | ATP binding cassette subfamily G member 2 (Junior blood group) | 118777 | 118762 | 163644 | 32, 30, 4 |
| 4 | ATP5I | ATP synthase, H+ transporting, mitochondrial Fo complex | 169020 | 133256 | 215375 | 19, 5, 43 |
| | | subunit E | | | | |
| 4 | ATP10D | ATPase phospholipid transporting 10D (putative) | 145246 | 169019 | 145244 | 35, 18, 11 |
| 4 | ATP8A1 | ATPase phospholipid transporting 8A1 | 124406 | 178343 | 215203 | 29, 26, 11 |
| 4 | BBS12 | Bardet-Biedl syndrome 12 | 181004 | 138684 | 138685 | 29, 4, 38 |
| 4 | BBS7 | Bardet-Biedl syndrome 7 | 138686 | 145386 | 138741 | 1, 45, 25 |
| 4 | BANK1 | B-cell scaffold protein with ankyrin repeats 1 | 153064 | 138814 | 138821 | |
| 4 | BEND4 | BEN domain containing 4 | 188848 | 014824 | 178343 | 22, 6, 39 |
| 4 | BTC | betacellulin | 174808 | 109321 | 169116 | 17, 27, 10 |
| 4 | BLOC1S4 | biogenesis of lysosomal organelles complex 1 subunit 4 | 186222 | 178988 | 170871 | 29, 32, 22 |
| 4 | BOD1L1 | biorientation of chromosomes in cell division 1 like 1 | 038219 | 109705 | 281620 | 1, 30, 26 |
| 4 | BMP2K | BMP2 inducible kinase | 138756 | 138772 | 163291 | 22, 40, 5 |
| 4 | BST1 | bone marrow stromal cell antigen 1 | 109743 | 237765 | 004468 | 43, 5, 32 |
| 4 | BMP3 | bone morphogenetic protein 3 | 152785 | 197826 | 138669 | 30, 7, 20 |
| 4 | BMPR1B | bone morphogenetic protein receptor type 1B | 138696 | 163110 | 182168 | 26, 27, 33 |
| 4 | C1QTNF7 | C1q and tumor necrosis factor related protein 7 | 163145 | 137449 | 048342 | 42, 25, 44 |
| 4 | CABS1 | calcium binding protein, spermatid associated 1 | 145309 | 171209 | 109208 | 38, 8, |
| 4 | CAMK2D | calcium/calmodulin dependent protein kinase II delta | 145349 | 145362 | 180801 | 25, 22, 29 |
| 4 | CLGN | calmegin | 153132 | 153130 | 205301 | 38, 30, 15 |
| 4 | CBR4 | carbonyl reductase 4 | 145439 | 129116 | 154447 | 42, 24, 18 |
| 4 | CPE | carboxypeptidase E | 109472 | 052802 | 038295 | 31, 28, 11 |
| 4 | CPZ | carboxypeptidase Z | 109625 | 155269 | 215612 | 43, 32, 27 |
| 4 | CSN1S1 | casein alpha s1 | 126545 | 109193 | 135222 | 10, |
| 4 | CSN2 | casein beta | 135222 | 126545 | 126549 | 10, |
| 4 | CSN3 | casein kappa | 171209 | 181617 | 145309 | 10, 8, |
| 4 | CASP3 | caspase 3 | 164305 | 168310 | 164306 | 13, 5, 11 |
| 4 | CASP6 | caspase 6 | 138794 | 005059 | 123739 | 3, 30, 4 |
| 4 | CTSO | cathepsin O | 256043 | 151790 | 145431 | 45, 1, 17 |
| 4 | CNOT6L | CCR4-NOT transcription complex subunit 6 like | 138767 | 156234 | 169288 | 2, 42, 22 |
| 4 | CD38 | CD38 molecule | 004468 | 109743 | 137440 | 22, 39, 36 |
| 4 | CISD2 | CDGSH iron sulfur domain 2 | 145354 | 109332 | 164037 | 2, 4, 1 |
| 4 | CDKN2AIP | CDKN2A interacting protein | 168564 | 185758 | 168556 | |
| 4 | CDS1 | CDP-diacylglycerol synthase 1 | 163624 | 163623 | 163625 | 29, 17, 32 |
| 4 | CWH43 | cell wall biogenesis 43 C-terminal homolog | 109182 | 145247 | 109184 | 26, 33, 30 |
| 4 | CENPC | centromere protein C | 145241 | 145242 | 035720 | 22, 26, 31 |
| 4 | CENPE | centromere protein E | 138778 | 164039 | 169836 | 22, 2, 12 |
| 4 | CENPU | centromere protein U | 151725 | 164306 | 151726 | |
| 4 | CEP135 | centrosomal protein 135 | 174799 | 090989 | 109265 | |
| 4 | CEP44 | centrosomal protein 44 | 164118 | 164117 | 164120 | 37, 23, 29 |
| 4 | CLCN3 | chloride voltage-gated channel 3 | 109572 | 137601 | 056050 | 24, 26, 11 |
| 4 | CCKAR | cholecystokinin A receptor | 163394 | 168214 | 109680 | 8, |
| 4 | CHRNA9 | cholinergic receptor nicotinic alpha 9 subunit | 174343 | 168421 | 163694 | |
| 4 | C4orf17 | chromosome 4 open reading frame 17 | 138813 | 196344 | 145331 | 38, 10, |
| 4 | C4orf19 | chromosome 4 open reading frame 19 | 154274 | 174145 | 181826 | 4, 29, 32 |
| 4 | C4orf22 | chromosome 4 open reading frame 22 | 197826 | 138675 | 152785 | 10, 38, 42 |
| 4 | C4orf26 | chromosome 4 open reading frame 26 | 174792 | 174796 | 138769 | 32, 4, 17 |
| 4 | C4orf3 | chromosome 4 open reading frame 3 | 164096 | 145390 | 145384 | 30, 29, 27 |
| 4 | C4orf32 | chromosome 4 open reading frame 32 | 174749 | 164093 | 138660 | 29, 21, 31 |
| 4 | C4orf33 | chromosome 4 open reading frame 33 | 151470 | 151466 | 138650 | 7, 4, 12 |
| 4 | C4orf36 | chromosome 4 open reading frame 36 | 163633 | 145283 | 172493 | 2, 32, 30 |
| 4 | C4orf45 | chromosome 4 open reading frame 45 | 164123 | 052795 | 109756 | 10, 38, |
| 4 | C4orf46 | chromosome 4 open reading frame 46 | 205208 | 171509 | 171503 | 18, 7, 9 |
| 4 | C4orf47 | chromosome 4 open reading frame 47 | 205129 | 109775 | 168491 | 10, 38, 32 |
| 4 | C4orf48 | chromosome 4 open reading frame 48 | 243449 | 185049 | 185818 | 28, 38, 14 |
| 4 | C4orf50 | chromosome 4 open reading frame 50 | 181215 | 072832 | 152969 | 39, |
| 4 | C4orf51 | chromosome 4 open reading frame 51 | 237136 | 151611 | 151612 | 10, |
| 4 | CFAP97 | cilia and flagella associated protein 97 | 164323 | 151729 | 109762 | 40, 29, 38 |
| 4 | CFAP99 | cilia and flagella associated protein 99 | 206113 | 159733 | 063978 | 38, 23, 17 |
| 4 | CLRN2 | clarin 2 | 249581 | 151552 | 002549 | |
| 4 | CLDN22 | claudin 22 | 177300 | 151718 | 185758 | |
| 4 | CLDN24 | claudin 24 | 185758 | 177300 | 168564 | |
| 4 | CLOCK | clock circadian regulator | 134852 | 134851 | 163440 | 29, 1, 25 |
| 4 | F11 | coagulation factor XI | 088926 | 164344 | 272297 | 19, 17, 4 |
| 4 | COQ2 | coenzyme Q2, polyprenyltransferase | 173085 | 145287 | 173083 | 9, 3, 28 |
| 4 | CC2D2A | coiled-coil and C2 domain containing 2A | 048342 | 163145 | 118564 | 26, 34, 40 |
| 4 | CCDC110 | coiled-coil domain containing 110 | 168491 | 205129 | 154553 | 11, 38, 4 |
| 4 | CCDC149 | coiled-coil domain containing 149 | 181982 | 109610 | 153012 | 34, 14, 33 |
| 4 | CCDC158 | coiled-coil domain containing 158 | 163749 | 118804 | 138771 | 38, 19, 5 |
| 4 | CCDC96 | coiled-coil domain containing 96 | 173013 | 132405 | 173011 | 38, 10, 27 |
| 4 | CCSER1 | coiled-coil serine rich protein 1 | 184305 | 138722 | 152208 | 31, 42, 38 |
| 4 | COL25A1 | collagen type XXV alpha 1 chain | 188517 | 164089 | 138802 | 9, 2, 38 |
| 4 | CRMP1 | collapsin response mediator protein 1 | 072832 | 072840 | 181215 | 8, 14, 26 |
| 4 | COMMD8 | COMM domain containing 8 | 169019 | 163288 | 145246 | 29, 4, 17 |
| 4 | CFI | complement factor I | 205403 | 123739 | 109534 | 4, 17, 19 |
| 4 | CPLX1 | complexin 1 | 168993 | 185619 | 178950 | 14, 8, 2 |
| 4 | COPS4 | COP9 signalosome subunit 4 | 138663 | 189308 | 145287 | 31, 29, 15 |
| 4 | CORIN | corin, serine peptidase | 145244 | 145246 | 170448 | 15, 44, 10 |
| 4 | COX18 | COX18, cytochrome c oxidase assembly factor | 163626 | 156140 | 132466 | 10, 29, 26 |
| 4 | CTBP1 | C-terminal binding protein 1 | 159692 | 159674 | 090316 | 11, 22, 1 |
| 4 | CXCL1 | C-X-C motif chemokine ligand 1 | 163739 | 109272 | 163737 | 45, 37, 10 |
| 4 | CXCL10 | C-X-C motif chemokine ligand 10 | 169245 | 156219 | 169248 | 29, 30, 44 |
| 4 | CXCL11 | C-X-C motif chemokine ligand 11 | 169248 | 169245 | 138750 | 24, 34, 7 |
| 4 | CXCL13 | C-X-C motif chemokine ligand 13 | 156234 | 138764 | 138767 | 30, 36, 23 |
| 4 | CXCL2 | C-X-C motif chemokine ligand 2 | 081041 | 163734 | 163738 | 19, 4, 37 |
| 4 | CXCL3 | C-X-C motif chemokine ligand 3 | 163734 | 163735 | 081041 | 9, 20, 4 |
| 4 | CXCL5 | C-X-C motif chemokine ligand 5 | 163735 | 163736 | 163734 | 20, 3, 45 |
| 4 | CXCL6 | C-X-C motif chemokine ligand 6 | 124875 | 169429 | 109272 | 45, 36, 4 |
| 4 | CXCL8 | C-X-C motif chemokine ligand 8 | 169429 | 169435 | 124875 | 5, 7, 45 |
| 4 | CXCL9 | C-X-C motif chemokine ligand 9 | 138755 | 280671 | 156219 | 36, 39, 37 |
| 4 | CXXC4 | CXXC finger protein 4 | 168772 | 169836 | 168769 | 1, 10, 42 |
| 4 | CNGA1 | cyclic nucleotide gated channel alpha 1 | 198515 | 163293 | 074966 | 15, 14, 25 |
| 4 | CCNA2 | cyclin A2 | 145386 | 123737 | 138686 | 9, 22, 39 |
| 4 | CDKL2 | cyclin dependent kinase like 2 | 138769 | 174792 | 138757 | 38, 30, 8 |
| 4 | GAK | cyclin G associated kinase | 178950 | 168993 | 127419 | 34, 29, 31 |
| 4 | CCNG2 | cyclin G2 | 138764 | 118816 | 156234 | 29, 1, 40 |
| 4 | CCNI | cyclin I | 118816 | 138758 | 138764 | 11, 41, 18 |
| 4 | CHIC2 | cysteine rich hydrophobic domain 2 | 109220 | 072201 | 180613 | 13, 38, 23 |
| 4 | CRIPAK | cysteine rich PAK1 inhibitor | 179979 | 163945 | 235608 | 13, 36, 39 |
| 4 | COX7B2 | cytochrome c oxidase subunit 7B2 | 170516 | 151834 | 109158 | 10, 38, 4 |
| 4 | CYP2U1 | cytochrome P450 family 2 subfamily U member 1 | 155016 | 164023 | 138796 | 39, 29, 35 |
| 4 | CYP4V2 | cytochrome P450 family 4 subfamily V member 2 | 145476 | 109794 | 164344 | 29, 11, 17 |
| 4 | CLNK | cytokine dependent hematopoietic cell linker | 109684 | 178163 | 002587 | 36, 5, 25 |
| 4 | CYTL1 | cytokine like 1 | 170891 | 163132 | 152953 | 10, 14, 6 |
| 4 | CPEB2 | cytoplasmic polyadenylation element binding protein 2 | 137449 | 281620 | 163145 | 45, 17, 23 |
| 4 | DCHS2 | dachsous cadherin-related 2 | 197410 | 145423 | 171566 | 27, 42, 25 |
| 4 | DCTD | dCMP deaminase | 129187 | 218336 | 151718 | 13, 9, 6 |
| 4 | DCAF16 | DDB1 and CUL4 associated factor 16 | 163257 | 047662 | 109805 | 11, 25, 4 |
| 4 | DCAF4L1 | DDB1 and CUL4 associated factor 4 like 1 | 182308 | 109133 | 014824 | 38, |
| 4 | DDX60L | DEAD-box helicase 60-like | 181381 | 137628 | 129116 | 1, 32, 25 |
| 4 | DHX15 | DEAH-box helicase 15 | 109606 | 109819 | 109610 | 11, 39, 24 |
| 4 | DTHD1 | death domain containing 1 | 197057 | 047365 | 174145 | 42, 30, 39 |
| 4 | DCUN1D4 | defective in cullin neddylation 1 domain containing 4 | 109184 | 109182 | 188993 | 34, 24, 26 |
| 4 | DEFB131 | defensin beta 131 | 186146 | 228856 | 109667 | |
| 4 | DMP1 | dentin matrix acidic phosphoprotein 1 | 152592 | 152591 | 029559 | 11, 6, 10 |
| 4 | DSPP | dentin sialophosphoprotein | 152591 | 152583 | 152592 | 17, |
| 4 | DCK | deoxycytidine kinase | 156136 | 173542 | 080493 | |
| 4 | DDX60 | DExD/H-box helicase 60 | 137628 | 109511 | 181381 | 44, 42, 39 |
| 4 | DGKQ | diacylglycerol kinase theta | 145214 | 127419 | 145217 | 31, 44, 26 |
| 4 | DKK2 | dickkopf WNT signaling pathway inhibitor 2 | 155011 | 250298 | 138801 | |
| 4 | DDIT4L | DNA damage inducible transcript 4 like | 145358 | 164032 | 164035 | 24, 15, 25 |
| 4 | POLN | DNA polymerase nu | 130997 | 185818 | 214367 | 29, 26, 2 |
| 4 | DNAJB14 | DnaJ heat shock protein family (Hsp40) member B14 | 164031 | 109270 | 164032 | 22, 30, 1 |
| 4 | DOK7 | docking protein 7 | 175920 | 109758 | 163956 | |
| 4 | DRD5 | dopamine receptor D5 | 169676 | 109667 | 071127 | 27, 14, 8 |
| 4 | DUX4 | double homeobox 4 | 260596 | 205097 | 151725 | |
| 4 | DCLK2 | doublecortin like kinase 2 | 170390 | 234828 | 198589 | 26, 29, 15 |
| 4 | DAPP1 | dual adaptor of phosphotyrosine and 3-phosphoinositides 1 | 070190 | 248713 | 109270 | 41, 30, 22 |
| 4 | ELF2 | E74 like ETS transcription factor 2 | 109381 | 151014 | 137463 | 26, 6, 22 |
| 4 | ENPP6 | ectonucleotide pyrophosphatase/phosphodiesterase 6 | 164303 | 173320 | 168310 | 26, 17, 31 |
| 4 | ETFDH | electron transfer flavoprotein dehydrogenase | 171503 | 205208 | 171497 | 31, 42, 21 |
| 4 | ELMOD2 | ELMO domain containing 2 | 179387 | 205301 | 109424 | 42, 2, 6 |
| 4 | ELOVL6 | ELOVL fatty acid elongase 6 | 170522 | 138798 | 138792 | 7, 4, 23 |
| 4 | ENAM | enamelin | 132464 | 178522 | 132465 | |
| 4 | ERVMER34-1 | Endogenous retrovirus group MER34 member 1 | 226887 | 109189 | 128045 | 32, 3, 17 |
| 4 | EMCN | endomucin | 164035 | 145358 | 138814 | 11, 43, 45 |
| 4 | EDNRA | endothelin receptor type A | 151617 | 137473 | 164168 | 44, 11, 31 |
| 4 | ENOPH1 | enolase-phosphatase 1 | 145293 | 152795 | 249242 | 13, 8, 29 |
| 4 | EPHA5 | EPH receptor A5 | 145242 | 205678 | 145241 | 8, 25, 20 |
| 4 | EGF | epidermal growth factor | 138798 | 183423 | 170522 | 10, 17, 31 |
| 4 | EREG | epiregulin | 124882 | 182585 | 109321 | 35, 3, 19 |
| 4 | EPGN | epithelial mitogen | 182585 | 163738 | 124882 | 13, 9, 44 |
| 4 | ETNPPL | ethanolamine-phosphate phospho-lyase | 164089 | 198856 | 188517 | 8, 26, 19 |
| 4 | EIF4E | eukaryotic translation initiation factor 4E | 151247 | 168785 | 164024 | 7, 4, 3 |
| 4 | EVC | EvC ciliary complex subunit 1 | 072840 | 173040 | 072832 | 23, 20, 30 |
| 4 | EVC2 | EvC ciliary complex subunit 2 | 173040 | 152953 | 072840 | 13, 41, 22 |
| 4 | EXOC1 | exocyst complex component 1 | 090989 | 250821 | 174799 | 4, 24, 42 |
| 4 | EXOSC9 | exosome component 9 | 123737 | 164112 | 145386 | 41, 6, 7 |
| 4 | FIP1L1 | factor interacting with PAPOLA and CPSF1 | 145216 | 184178 | 282278 | 5, 40, 18 |
| 4 | FAM47E- | FAM47E-STBD1 readthrough | 272414 | 189157 | 118804 | 26, 15, 28 |
| | STBD1 | | | | | |
| 4 | FAM114A1 | family with sequence similarity 114 member A1 | 197712 | 174130 | 121895 | 42, 6, 11 |
| 4 | FAM13A | family with sequence similarity 13 member A | 138640 | 177432 | 180346 | 32, 42, 8 |
| 4 | FAM149A | family with sequence similarity 149 member A | 109794 | 164342 | 145476 | 26, 17, 37 |
| 4 | FAM160A1 | family with sequence similarity 160 member A1 | 164142 | 189099 | 059691 | 18, 23, 24 |
| 4 | FAM175A | family with sequence similarity 175 member A | 163322 | 163319 | 138678 | 34, 45, 22 |
| 4 | FAM184B | family with sequence similarity 184 member B | 047662 | 118579 | 163257 | 24, 15, 8 |
| 4 | FAM193A | family with sequence similarity 193 member A | 125386 | 063978 | 168884 | 26, 18, 22 |
| 4 | FAM198B | family with sequence similarity 198 member B | 164125 | 120251 | 164124 | 43, 45, 2 |
| 4 | FAM200B | family with sequence similarity 200 member B | 237765 | 118564 | 109743 | 34, 25, 8 |
| 4 | FAM218A | family with sequence similarity 218 member A | 250486 | 183439 | 176979 | 26, 4, 28 |
| 4 | FAM47E | family with sequence similarity 47 member E | 189157 | 138760 | 272414 | 26, 15, 28 |
| 4 | FAM53A | family with sequence similarity 53 member A | 174137 | 235608 | 163950 | 31, 38, 37 |
| 4 | FAM90A26 | Family with sequence similarity 90 member A26 | 229924 | 215612 | 231396 | |
| 4 | FAT1 | FAT atypical cadherin 1 | 083857 | 168412 | 179059 | 26, 13, 23 |
| 4 | FAT4 | FAT atypical cadherin 4 | 196159 | 151458 | 164066 | 1, 14, 35 |
| 4 | FABP2 | fatty acid binding protein 2 | 145384 | 164096 | 138735 | 16, 10, 17 |
| 4 | FBXL5 | F-box and leucine rich repeat protein 5 | 118564 | 048342 | 237765 | 31, 13, 11 |
| 4 | FBXW7 | F-box and WD repeat domain containing 7 | 109670 | 059691 | 170006 | 29, 44, 23 |
| 4 | FBXO8 | F-box protein 8 | 164117 | 164107 | 164118 | 31, 28, 17 |
| 4 | FHDC1 | FH2 domain containing 1 | 137460 | 164144 | 109654 | 32, 15, 29 |
| 4 | FGA | fibrinogen alpha chain | 171560 | 171564 | 171557 | 19, 5, 36 |
| 4 | FGB | fibrinogen beta chain | 171564 | 171566 | 171560 | 19, 5, 15 |
| 4 | FGG | fibrinogen gamma chain | 171557 | 171560 | 121207 | 19, 25, 5 |
| 4 | FGF2 | fibroblast growth factor 2 | 138685 | 181004 | 170917 | 40, 44, 37 |
| 4 | FGF5 | fibroblast growth factor 5 | 138675 | 152784 | 197826 | 35, 6, 17 |
| 4 | FGFBP1 | fibroblast growth factor binding protein 1 | 137440 | 004468 | 137441 | 37, 22, 24 |
| 4 | FGFBP2 | fibroblast growth factor binding protein 2 | 137441 | 137440 | 007062 | 6, 10, 2 |
| 4 | FGFR3 | fibroblast growth factor receptor 3 | 068078 | 013810 | 168924 | 18, 12, 26 |
| 4 | FGFRL1 | fibroblast growth factor receptor-like 1 | 127418 | 127415 | 178222 | 2, 33, 27 |
| 4 | FDCSP | follicular dendritic cell secreted protein | 181617 | 109205 | 171209 | 30, 45, 22 |
| 4 | FNIP2 | folliculin interacting protein 2 | 052795 | 171497 | 164123 | |
| 4 | FSTL5 | follistatin like 5 | 168843 | 109756 | 145414 | 11, 8, 31 |
| 4 | FREM3 | FRAS1 related extracellular matrix 3 | 183090 | 153147 | 197465 | 20, 8, |
| 4 | FRAS1 | Fraser extracellular matrix complex subunit 1 | 138759 | 169288 | 138772 | 40, 20, 12 |
| 4 | FRYL | FRY like transcription coactivator | 075539 | 182223 | 109180 | 11, 29, 26 |
| 4 | FRG1 | FSHD region gene 1 | 109536 | 184108 | 205097 | 7, 30, 31 |
| 4 | FRG2 | FSHD region gene 2 | 205097 | 109536 | 260596 | |
| 4 | GPR78 | G protein-coupled receptor 78 | 155269 | 155275 | 109625 | 32, 25, 8 |
| 4 | GRK4 | G protein-coupled receptor kinase 4 | 125388 | 087269 | 197386 | 38, 2, 29 |
| 4 | G3BP2 | G3BP stress granule assembly factor 2 | 138757 | 138769 | 138768 | 42, 40, 12 |
| 4 | GABRA2 | gamma-aminobutyric acid type A receptor alpha2 subunit | 151834 | 163285 | 170516 | 8, 10, 20 |
| 4 | GABRA4 | gamma-aminobutyric acid type A receptor alpha4 subunit | 109158 | 170516 | 163288 | 8, 10, 45 |
| 4 | GABRB1 | gamma-aminobutyric acid type A receptor beta1 subunit | 163288 | 109158 | 169019 | 8, 15, 17 |
| 4 | GABRG1 | gamma-aminobutyric acid type A receptor gamma1 subunit | 163285 | 163281 | 151834 | 8, 14, |
| 4 | GAR1 | GAR1 ribonucleoprotein | 109534 | 205403 | 180245 | 3, 21, 6 |
| 4 | GC | GC, vitamin D binding protein | 145321 | 080493 | 056291 | 19, 28, 23 |
| 4 | GIMD1 | GIMAP family P-loop NTPase domain containing 1 | 250298 | 164022 | 155011 | |
| 4 | GNPDA2 | glucosamine-6-phosphate deaminase 2 | 163281 | 151806 | 163285 | 4, 31, 25 |
| 4 | GBA3 | glucosylceramidase beta 3 (gene/pseudogene) | 249948 | 152990 | 109819 | |
| 4 | GRIA2 | glutamate ionotropic receptor AMPA type subunit 2 | 120251 | 109738 | 164125 | 8, 31, 44 |
| 4 | GRID2 | glutamate ionotropic receptor delta type subunit 2 | 152208 | 184305 | 172238 | 38, 10, 8 |
| 4 | ENPEP | glutamyl aminopeptidase | 138792 | 170522 | 164093 | 17, 29, 32 |
| 4 | GATB | glutamyl-tRNA amidotransferase subunit B | 059691 | 164142 | 109670 | 5, 16, 8 |
| 4 | GRXCR1 | glutaredoxin and cysteine rich domain containing 1 | 215203 | 124406 | 183783 | 10, 33, |
| 4 | GSTCD | glutathione S-transferase C-terminal domain containing | 138780 | 138785 | 168743 | 22, 23, 13 |
| 4 | GK2 | glycerol kinase 2 | 196475 | 156269 | 163297 | 38, 8, 33 |
| 4 | GK3P | Glycerol kinase 3 pseudogene | 229894 | 109466 | 052802 | 38, 23, 8 |
| 4 | GPAT3 | glycerol-3-phosphate acyltransferase 3 | 138678 | 163322 | 163623 | 17, 13, 26 |
| 4 | GLRA3 | glycine receptor alpha 3 | 145451 | 164120 | 168594 | 38, |
| 4 | GLRB | glycine receptor beta | 109738 | 145431 | 120251 | 8, 26, 31 |
| 4 | GYPA | glycophorin A (MNS blood group) | 170180 | 250361 | 164161 | 34, 7, 5 |
| 4 | GYPB | glycophorin B (MNS blood group) | 250361 | 197465 | 170180 | 34, 7, 5 |
| 4 | GYPE | glycophorin E (MNS blood group) | 197465 | 183090 | 250361 | |
| 4 | GPM6A | glycoprotein M6A | 150625 | 168594 | 150627 | 8, |
| 4 | GNRHR | gonadotropin releasing hormone receptor | 109163 | 033178 | 153802 | 31, 2, |
| 4 | GPRIN3 | GPRIN family member 3 | 185477 | 180346 | 145335 | 22, 23, 42 |
| 4 | GAB1 | GRB2 associated binding protein 1 | 109458 | 170185 | 153147 | 31, 13, 8 |
| 4 | GRSF1 | G-rich RNA sequence binding factor 1 | 132463 | 018189 | 173542 | 21, 29, 22 |
| 4 | GRPEL1 | GrpE like 1, mitochondrial | 109519 | 173011 | 184985 | 2, 30, 11 |
| 4 | GSX2 | GS homeobox 2 | 180613 | 109220 | 134853 | 22, 8, |
| 4 | GUCY1A3 | guanylate cyclase 1 soluble subunit alpha | 164116 | 164114 | 061918 | 33, 15, 14 |
| 4 | GUCY1B3 | guanylate cyclase 1 soluble subunit beta | 061918 | 164116 | 256394 | 8, 17, 32 |
| 4 | GUF1 | GUF1 homolog, GTPase | 151806 | 177752 | 163281 | 42, 7, 13 |
| 4 | H2AFZ | H2A histone family member Z | 164032 | 164031 | 145358 | 7, 5, 29 |
| 4 | HMX1 | H6 family homeobox 1 | 215612 | 109625 | 229924 | 14, 8, 20 |
| 4 | HAUS3 | HAUS augmin like complex subunit 3 | 214367 | 130997 | 123933 | 39, 29, 38 |
| 4 | HAND2 | heart and neural crest derivatives expressed 2 | 164107 | 164106 | 164117 | 27, 26, 2 |
| 4 | HSPA4L | heat shock protein family A (Hsp70) member 4 like | 164070 | 151475 | 142731 | 38, 42, 4 |
| 4 | HERC3 | HECT and RLD domain containing E3 ubiquitin protein ligase 3 | 138641 | 145337 | 255072 | 42, 7, 1 |
| 4 | HERC5 | HECT and RLD domain containing E3 ubiquitin protein ligase 5 | 138646 | 138642 | 145337 | 18, 31, 10 |
| 4 | HERC6 | HECT and RLD domain containing E3 ubiquitin protein ligase | 138642 | 163644 | 138646 | 42, 1, 9 |
| | | family member 6 | | | | |
| 4 | HHIP | hedgehog interacting protein | 164161 | 170180 | 164162 | 33, |
| 4 | HELQ | helicase, POLQ-like | 163312 | 173083 | 163319 | 45, 42, 44 |
| 4 | HELT | helt bHLH transcription factor | 187821 | 151726 | 151729 | 27, |
| 4 | HPGDS | hematopoietic prostaglandin D synthase | 163106 | 163104 | 163110 | 10, 32, 30 |
| 4 | HS3ST1 | heparan sulfate-glucosamine 3-sulfotransferase 1 | 002587 | 109684 | 157869 | 27, 30, 20 |
| 4 | HPSE | heparanase | 173083 | 173085 | 163312 | 13, 32, 26 |
| 4 | HNRNPD | heterogeneous nuclear ribonucleoprotein D | 138668 | 138670 | 152795 | 11, 12, 6 |
| 4 | HNRNPDL | heterogeneous nuclear ribonucleoprotein D like | 152795 | 138668 | 145293 | |
| 4 | HGFAC | HGF activator | 109758 | 159788 | 175920 | 19, 27, 38 |
| 4 | HMGB2 | high mobility group box 2 | 164104 | 109586 | 164105 | 41, 21, 6 |
| 4 | HTN1 | histatin 1 | 126550 | 205649 | 187533 | 34, 25, 38 |
| 4 | HTN3 | histatin 3 | 205649 | 279857 | 126550 | 34, 25, 45 |
| 4 | HPF1 | histone PARylation factor 1 | 056050 | 109572 | 198948 | |
| 4 | HOPX | HOP homeobox | 171476 | 249693 | 128040 | 40, 10, 35 |
| 4 | HTRA3 | HtrA serine peptidase 3 | 170801 | 281359 | 087008 | 15, 13, 34 |
| 4 | HTT | huntingtin | 197386 | 125388 | 188981 | 18, 29, 34 |
| 4 | HADH | hydroxyacyl-CoA dehydrogenase | 138796 | 155016 | 138795 | 15, 3, 16 |
| 4 | HPGD | hydroxyprostaglandin dehydrogenase 15-(NAD) | 164120 | 164118 | 145451 | 4, 32, 44 |
| 4 | HSD17B11 | hydroxysteroid 17-beta dehydrogenase 11 | 198189 | 170509 | 170502 | 4, 7, 6 |
| 4 | HSD17B13 | hydroxysteroid 17-beta dehydrogenase 13 | 170509 | 145332 | 198189 | 19, 42, 23 |
| 4 | IDUA | iduronidase, alpha-L- | 127415 | 145217 | 127418 | 5, 20, 28 |
| 4 | ING2 | inhibitor of growth family member 2 | 168556 | 168564 | 182552 | 11, 33, 22 |
| 4 | INPP4B | inositol polyphosphate-4-phosphatase type II B | 109452 | 164136 | 170185 | 23, 35, 43 |
| 4 | IGFBP7 | insulin like growth factor binding protein 7 | 163453 | 047315 | 150471 | |
| 4 | INTS12 | integrator complex subunit 12 | 138785 | 236699 | 138780 | 29, 30, 7 |
| 4 | IBSP | integrin binding sialoprotein | 029559 | 152592 | 152595 | 11, 6, 10 |
| 4 | IRF2 | interferon regulatory factor 2 | 168310 | 164303 | 164305 | 24, 16, 5 |
| 4 | IL15 | interleukin 15 | 164136 | 109445 | 109452 | 18, 1, 45 |
| 4 | IL2 | interleukin 2 | 109471 | 164113 | 138684 | 5, |
| 4 | IL21 | interleukin 21 | 138684 | 109471 | 181004 | 38, |
| 4 | INTU | inturned planar cell polarity protein | 164066 | 196159 | 151475 | 31, 45, 42 |
| 4 | JADE1 | jade family PHD finger 1 | 077684 | 164040 | 151466 | 42, 23, 43 |
| 4 | JAKMIP1 | janus kinase and microtubule interacting protein 1 | 152969 | 181215 | 109501 | 8, 14, 6 |
| 4 | JCHAIN | joining chain of multimeric IgA and IgM | 132465 | 132464 | 132467 | |
| 4 | KLKB1 | kallikrein B1 | 164344 | 145476 | 088926 | 19, 29, 28 |
| 4 | KLHL2 | kelch like family member 2 | 109466 | 170088 | 229894 | 40, 37, 8 |
| 4 | KLHL5 | kelch like family member 5 | 109790 | 121895 | 157796 | 22, 33, 30 |
| 4 | KLHL8 | kelch like family member 8 | 145332 | 172493 | 170509 | |
| 4 | KIAA0232 | KIAA0232 | 170871 | 186222 | 132405 | 26, 43, 29 |
| 4 | KIAA0922 | KIAA0922 | 121210 | 121211 | 137462 | 22, 41, 7 |
| 4 | KIAA1109 | KIAA1109 | 138688 | 138741 | 164113 | 29, 1, 25 |
| 4 | KIAA1211 | KIAA1211 | 109265 | 174799 | 157426 | 31, 38, 22 |
| 4 | KDR | kinase insert domain receptor | 128052 | 157404 | 128039 | 31, 29, 11 |
| 4 | KIT | KIT proto-oncogene receptor tyrosine kinase | 157404 | 134853 | 128052 | 27, 42, 1 |
| 4 | KLB | klotho beta | 134962 | 035928 | 163682 | 1, 23, 38 |
| 4 | KLF3 | Kruppel like factor 3 | 109787 | 250254 | 174123 | 29, 44, 16 |
| 4 | LARP1B | La ribonucleoprotein domain family member 1B | 138709 | 164074 | 164040 | 10, 34, 45 |
| 4 | LARP7 | La ribonucleoprotein domain family member 7 | 174720 | 138658 | 145362 | 7, 12, 31 |
| 4 | LAMTOR3 | late endosomal/lysosomal adaptor, MAPK and MTOR activator 3 | 109270 | 070190 | 164031 | 29, 4, 34 |
| 4 | LRPAP1 | LDL receptor related protein associated protein 1 | 163956 | 175920 | 280230 | 29, 1, 34 |
| 4 | LRAT | lecithin retinol acyltransferase (phosphatidylcholine--retinol | 121207 | 171557 | 151962 | |
| | | O-acyltransferase) | | | | |
| 4 | LAP3 | leucine aminopeptidase 3 | 002549 | 249581 | 118579 | 13, 4, 30 |
| 4 | LRRC66 | leucine rich repeat containing 66 | 188993 | 109184 | 163069 | 31, 6, 38 |
| 4 | LGI2 | leucine rich repeat LGI family member 2 | 153012 | 181982 | 109618 | 29, 33, 12 |
| 4 | LRIT3 | leucine rich repeat, Ig-like and transmembrane domains 3 | 183423 | 180245 | 138798 | 14, 8, |
| 4 | LETM1 | leucine zipper and EF-hand containing transmembrane protein 1 | 168924 | 068078 | 109685 | 3, 34, 1 |
| 4 | LCORL | ligand dependent nuclear receptor corepressor like | 178177 | 109805 | 145147 | 2, 11, 3 |
| 4 | LNX1 | ligand of numb-protein X 1 | 072201 | 282278 | 109220 | |
| 4 | LIMCH1 | LIM and calponin homology domains 1 | 064042 | 154277 | 109132 | |
| 4 | LDB2 | LIM domain binding 2 | 169744 | 169762 | 151552 | 8, 36, 45 |
| 4 | LIN54 | lin-54 DREAM MuvB core complex component | 189308 | 168152 | 138663 | 42, 30, 40 |
| 4 | LIAS | lipoic acid synthetase | 121897 | 163682 | 109814 | 4, 1, 26 |
| 4 | LINC01207 | Long intergenic non-protein coding RNA 1207 | 248771 | 145416 | 248329 | 24, 33, 42 |
| 4 | LRBA | LPS responsive beige-like anchor protein | 198589 | 170390 | 181541 | 11, 37, 29 |
| 4 | LRP2BP | LRP2 binding protein | 109771 | 109762 | 186352 | 38, 42, 23 |
| 4 | LSM6 | LSM6 homolog, U6 small nuclear RNA and mRNA degradation | 164167 | 151612 | 250673 | 41, 22, 30 |
| | | associated | | | | |
| 4 | LYAR | Ly1 antibody reactive | 145220 | 132406 | 168826 | 3, 13, 38 |
| 4 | LEF1 | lymphoid enhancer binding factor 1 | 138795 | 138796 | 109475 | 39, 35, 11 |
| 4 | MAB21L2 | mab-21 like 2 | 181541 | 198589 | 145425 | 14, 24, 25 |
| 4 | MAEA | macrophage erythroblast attacher | 090316 | 159692 | 163945 | |
| 4 | MAD2L1 | MAD2 mitotic arrest deficient-like 1 (yeast) | 164109 | 138735 | 138738 | 7, 9, 21 |
| 4 | MFSD10 | major facilitator superfamily domain containing 10 | 109736 | 087274 | 087269 | 41, 26, 9 |
| 4 | MFSD7 | major facilitator superfamily domain containing 7 | 169026 | 215375 | 185619 | 23, 27, 9 |
| 4 | MFSD8 | major facilitator superfamily domain containing 8 | 164073 | 142731 | 164074 | 31, 26, 11 |
| 4 | MAN2B2 | mannosidase alpha class 2B member 2 | 013288 | 074211 | 179010 | 7, 22, 40 |
| 4 | MANBA | mannosidase beta | 109323 | 109320 | 109332 | 31, 39, 40 |
| 4 | MAML3 | mastermind like transcriptional coactivator 3 | 196782 | 085871 | 153130 | 22, 24, 42 |
| 4 | MEPE | matrix extracellular phosphoglycoprotein | 152595 | 029559 | 118785 | 6, 32, 8 |
| 4 | MXD4 | MAX dimerization protein 4 | 123933 | 214367 | 159733 | 26, 1, 6 |
| 4 | MED28 | mediator complex subunit 28 | 118579 | 002549 | 047662 | 43, 41, 12 |
| 4 | MND1 | meiotic nuclear divisions 1 | 121211 | 109654 | 121210 | 41, 6, 37 |
| 4 | MTNR1A | melatonin receptor 1A | 168412 | 272297 | 083857 | 38, 19, |
| 4 | MARCH1 | membrane associated ring-CH-type finger 1 | 145416 | 198498 | 248771 | |
| 4 | METAP1 | methionyl aminopeptidase 1 | 164024 | 151247 | 197894 | 13, 18, 37 |
| 4 | MTHFD2L | methylenetetrahydrofolate dehydrogenase (NADP+ dependent) | 163738 | 081041 | 182585 | |
| | | 2-like | | | | |
| 4 | MMAA | methylmalonic aciduria (cobalamin deficiency) cblA type | 151611 | 170365 | 237136 | 10, 7, 42 |
| 4 | MSMO1 | methylsterol monooxygenase 1 | 052802 | 229894 | 109472 | 34, 29, 2 |
| 4 | METTL14 | methyltransferase like 14 | 145388 | 164099 | 150961 | 30, 1, 43 |
| 4 | MGAT4D | MGAT4 family member D | 205301 | 153132 | 179387 | |
| 4 | MFAP3L | microfibrillar associated protein 3 like | 198948 | 056050 | 109576 | |
| 4 | MGST2 | microsomal glutathione S-transferase 2 | 085871 | 145391 | 196782 | 29, 44, 31 |
| 4 | MTTP | microsomal triglyceride transfer protein | 138823 | 145331 | 248713 | |
| 4 | MAP9 | microtubule associated protein 9 | 164114 | 185149 | 164116 | 29, 45, 1 |
| 4 | MGARP | mitochondria localized glutamic acid rich protein | 137463 | 109381 | 109390 | 14, 2, 44 |
| 4 | MCUB | mitochondrial calcium uniporter dominant negative beta subunit | 005059 | 138802 | 138794 | 34, 22, 26 |
| 4 | MRPL1 | mitochondrial ribosomal protein L1 | 169288 | 138767 | 138759 | 29, 10, 4 |
| 4 | MRPS18C | mitochondrial ribosomal protein S18C | 163319 | 163312 | 163322 | 29, 45, 7 |
| 4 | MAPK10 | mitogen-activated protein kinase 10 | 109339 | 138639 | 163629 | |
| 4 | MOB1B | MOB kinase activator 1B | 173542 | 132463 | 156136 | 1, 22, 17 |
| 4 | MRFAP1 | Morf4 family associated protein 1 | 179010 | 013288 | 163993 | 31, 33, 12 |
| 4 | MRFAP1L1 | Morf4 family associated protein 1 like 1 | 178988 | 163993 | 186222 | 36, 15, 39 |
| 4 | MSX1 | msh homeobox 1 | 163132 | 168818 | 170891 | 44, 12, 15 |
| 4 | MTRNR2L13 | MT-RNR2-like 13 | 270394 | 138653 | 174599 | |
| 4 | MUC7 | mucin 7, secreted | 171195 | 171199 | 187689 | 42, 34, 25 |
| 4 | MMRN1 | multimerin 1 | 138722 | 145335 | 184305 | 45, 43, 42 |
| 4 | MSANTD1 | Myb/SANT DNA binding domain containing 1 | 188981 | 197386 | 159788 | 36, 28, 14 |
| 4 | MYL5 | myosin light chain 5 | 215375 | 169020 | 169026 | |
| 4 | MYOZ2 | myozenin 2 | 172399 | 172403 | 145390 | 25, 15, 18 |
| 4 | NAA11 | N(alpha)-acetyltransferase 11, NatA catalytic subunit | 156269 | 163291 | 196475 | 35, 32, 38 |
| 4 | NAA15 | N(alpha)-acetyltransferase 15, NatA auxiliary subunit | 164134 | 109390 | 172007 | 13, 6, 45 |
| 4 | NAT8L | N-acetyltransferase 8 like | 185818 | 243449 | 130997 | 1, 31, 26 |
| 4 | NWD2 | NACHT and WD repeat domain containing 2 | 174145 | 197057 | 154274 | |
| 4 | NAAA | N-acylethanolamine acid amidase | 138744 | 156194 | 198301 | 5, 14, 17 |
| 4 | NDUFC1 | NADH: ubiquinone oxidoreductase subunit C1 | 109390 | 137463 | 164134 | 31, 17, 3 |
| 4 | NDST3 | N-deacetylase and N-sulfotransferase 3 | 164100 | 174599 | 164099 | 5, 39, 2 |
| 4 | NDST4 | N-deacetylase and N-sulfotransferase 4 | 138653 | 174607 | 270394 | 20, |
| 4 | N4BP2 | NEDD4 binding protein 2 | 078177 | 121892 | 168421 | 39, 12, 34 |
| 4 | NELFA | negative elongation factor complex member A | 185049 | 109685 | 243449 | 38, 39, 42 |
| 4 | NEIL3 | nei like DNA glycosylase 3 | 109674 | 150630 | 038002 | 41, 43, 34 |
| 4 | NPNT | nephronectin | 168743 | 138780 | 145348 | 11, 40, 45 |
| 4 | NEUROG2 | neurogenin 2 | 178403 | 073331 | 138658 | 8, 38, |
| 4 | NMU | neuromedin U | 109255 | 163440 | 250821 | 18, 27, 10 |
| 4 | NDNF | neuron derived neurotrophic factor | 173376 | 138738 | 050730 | 11, 14, 6 |
| 4 | NSG1 | Neuron-specific protein family member 1 | 168824 | 168826 | 168818 | 8, 31, 17 |
| 4 | NPFFR2 | neuropeptide FF receptor 2 | 056291 | 145321 | 156140 | 32, 10, 6 |
| 4 | NPY1R | neuropeptide Y receptor Y1 | 164128 | 145414 | 164129 | 23, 6, 36 |
| 4 | NPY2R | neuropeptide Y receptor Y2 | 185149 | 151962 | 164114 | 26, 8, |
| 4 | NPY5R | neuropeptide Y receptor Y5 | 164129 | 164128 | 151005 | |
| 4 | NEK1 | NIMA related kinase 1 | 137601 | 154447 | 109572 | 19, 11, 7 |
| 4 | NIPAL1 | NIPA like domain containing 1 | 163293 | 170448 | 198515 | 24, 32, 40 |
| 4 | NOA1 | nitric oxide associated 1 | 084092 | 084093 | 047315 | 39, 36, 22 |
| 4 | NKX1-1 | NK1 homeobox 1 | 235608 | 179979 | 174137 | |
| 4 | NKX3-2 | NK3 homeobox 2 | 109705 | 157869 | 038219 | 27, 38, 12 |
| 4 | NKX6-1 | NK6 homeobox 1 | 163623 | 138678 | 163624 | 28, 27, |
| 4 | NOCT | nocturnin | 151014 | 151012 | 109381 | 25, 4, 10 |
| 4 | NCAPG | non-SMC condensin I complex subunit G | 109805 | 163257 | 178177 | 9, 39, 10 |
| 4 | NOP14 | NOP14 nucleolar protein | 087269 | 109736 | 125388 | 21, 29, 26 |
| 4 | NSUN7 | NOP2/Sun RNA methyltransferase family member 7 | 179299 | 163694 | 163697 | 42, 38, 12 |
| 4 | NAF1 | nuclear assembly factor 1 ribonucleoprotein | 145414 | 168843 | 164128 | 37, 7, 42 |
| 4 | NFKB1 | nuclear factor kappa B subunit 1 | 109320 | 138821 | 109323 | 26, 42, 7 |
| 4 | NR3C2 | nuclear receptor subfamily 3 group C member 2 | 151623 | 071205 | 234828 | 37, 14, 34 |
| 4 | NFXL1 | nuclear transcription factor, X-box binding like 1 | 170448 | 145244 | 163293 | 7, 11, 45 |
| 4 | NUP54 | nucleoporin 54 | 138750 | 169248 | 138760 | |
| 4 | NAP1L5 | nucleosome assembly protein 1 like 5 | 177432 | 255072 | 138640 | 11, 31, 8 |
| 4 | NUDT6 | nudix hydrolase 6 | 170917 | 138685 | 145375 | 22, |
| 4 | NUDT9 | nudix hydrolase 9 | 170502 | 198189 | 152583 | 17, 26, 3 |
| 4 | OCIAD1 | OCIA domain containing 1 | 109180 | 075539 | 145247 | 24, 11, 31 |
| 4 | OCIAD2 | OCIA domain containing 2 | 145247 | 109180 | 109182 | 29, 45, 3 |
| 4 | ODAM | odontogenic, ameloblast asssociated | 109205 | 187533 | 181617 | 10, 42, 15 |
| 4 | OSTC | oligosaccharyltransferase complex non-catalytic subunit | 198856 | 109475 | 164089 | 34, 4, 7 |
| 4 | OPRPN | opiorphin prepropeptide | 171199 | 171201 | 171195 | 14, 10, |
| 4 | OTOP1 | otopetrin 1 | 163982 | 184160 | 132406 | |
| 4 | OTUD4 | OTU deubiquitinase 4 | 164164 | 164163 | 170365 | 1, 22, 25 |
| 4 | PHOX2B | paired like homeobox 2b | 109132 | 064042 | 109133 | 33, 12, 20 |
| 4 | PITX2 | paired like homeodomain 2 | 164093 | 138792 | 174749 | 32, 31, 24 |
| 4 | PALLD | palladin, cytoskeletal associated protein | 129116 | 181381 | 145439 | 43, 1, 44 |
| 4 | PACRGL | PARK2 coregulated like | 163138 | 145147 | 185774 | 36, 4, 38 |
| 4 | PDS5A | PDS5 cohesin associated factor A | 121892 | 078140 | 078177 | 11, 7, 6 |
| 4 | PDLIM3 | PDZ and LIM domain 3 | 154553 | 168491 | 154556 | 15, 25, 37 |
| 4 | PDLIM5 | PDZ and LIM domain 5 | 163110 | 163106 | 138696 | 25, 15, 45 |
| 4 | PPID | peptidylprolyl isomerase D | 171497 | 171503 | 052795 | 40, 15, 9 |
| 4 | PDCL2 | phosducin like 2 | 163440 | 134852 | 109255 | 38, 5, 10 |
| 4 | PI4K2B | phosphatidylinositol 4-kinase type 2 beta | 038210 | 281028 | 168228 | 42, 18, 11 |
| 4 | PIGG | phosphatidylinositol glycan anchor biosynthesis class G | 174227 | 182903 | 133256 | 39, |
| 4 | PIGY | phosphatidylinositol glycan anchor biosynthesis class Y | 255072 | 138641 | 177432 | 31, 34, 13 |
| 4 | PDE5A | phosphodiesterase 5A | 138735 | 145384 | 164109 | 45, 11, 42 |
| 4 | PDE6B | phosphodiesterase 6B | 133256 | 174227 | 169020 | 39, 26, 14 |
| 4 | PGM2 | phosphoglucomutase 2 | 169299 | 181826 | 279386 | 13, 43, 4 |
| 4 | PLA2G12A | phospholipase A2 group XIIA | 123739 | 138794 | 205403 | 29, 34, 31 |
| 4 | PPAT | phosphoribosyl pyrophosphate amidotransferase | 128059 | 157426 | 128050 | 42, 13, 21 |
| 4 | PAICS | phosphoribosylaminoimidazole carboxylase and | 128050 | 128059 | 174780 | 31, 3, 12 |
| | | phosphoribosylaminoimidazolesuccinocarboxamide synthase | | | | |
| 4 | PYURF | PIGY upstream reading frame | 145337 | 138646 | 138641 | |
| 4 | PTTG2 | pituitary tumor-transforming 2 | 250254 | 065882 | 109787 | 44, |
| 4 | PLAC8 | placenta specific 8 | 145287 | 138663 | 173085 | 37, 7, 36 |
| 4 | PDGFC | platelet derived growth factor C | 145431 | 256043 | 109738 | 29, 11, 6 |
| 4 | PDGFRA | platelet derived growth factor receptor alpha | 134853 | 180613 | 157404 | 11, 1, 29 |
| 4 | PF4 | platelet factor 4 | 163737 | 163739 | 163736 | 15, 7, 16 |
| 4 | PF4V1 | platelet factor 4 variant 1 | 109272 | 124875 | 163739 | |
| 4 | PLRG1 | pleiotropic regulator 1 | 171566 | 197410 | 171564 | 13, 43, 7 |
| 4 | PLK4 | polo like kinase 4 | 142731 | 164070 | 164073 | 38, 7, 12 |
| 4 | PABPC4L | poly(A) binding protein cytoplasmic 4 like | 254535 | 138650 | 189184 | 24, 7, 36 |
| 4 | PCGF3 | polycomb group ring finger 3 | 185619 | 169026 | 168993 | 43, 13, 26 |
| 4 | PKD2 | polycystin 2, transient receptor potential cation channel | 118762 | 118785 | 118777 | 11, 28, 43 |
| 4 | GALNT7 | polypeptide N-acetylgalactosaminyltransferase 7 | 109586 | 174473 | 164104 | 42, 39, 24 |
| 4 | GALNTL6 | polypeptide N-acetylgalactosaminyltransferase-like 6 | 174473 | 109576 | 109586 | 35, 38, 17 |
| 4 | KCTD8 | potassium channel tetramerization domain containing 8 | 183783 | 215203 | 177752 | 40, 8, 38 |
| 4 | KCNIP4 | potassium voltage-gated channel interacting protein 4 | 185774 | 163138 | 152990 | 26, 17, 29 |
| 4 | POU4F2 | POU class 4 homeobox 2 | 151615 | 120519 | 137473 | 14, 38, |
| 4 | PPARGC1A | PPARG coactivator 1 alpha | 109819 | 249948 | 109606 | |
| 4 | PRDM5 | PR/SET domain 5 | 138738 | 164109 | 173376 | |
| 4 | PRDM8 | PR/SET domain 8 | 152784 | 163297 | 138675 | 14, 8, 2 |
| 4 | PRIMPOL | primase and DNA directed polymerase | 164306 | 164305 | 151725 | 4, 10, 15 |
| 4 | PGRMC2 | progesterone receptor membrane component 2 | 164040 | 138709 | 077684 | 29, 43, 31 |
| 4 | PAQR3 | progestin and adipoQ receptor family member 3 | 163291 | 138756 | 156269 | 43, 4, 1 |
| 4 | PRR27 | proline rich 27 | 187533 | 126550 | 109205 | |
| 4 | PROM1 | prominin 1 | 007062 | 137441 | 169762 | |
| 4 | PPBP | pro-platelet basic protein | 163736 | 163737 | 163735 | 5, 15, 32 |
| 4 | PSAPL1 | prosaposin-like 1 (gene/pseudogene) | 178597 | 184985 | 196526 | 35, 38, 8 |
| 4 | PARM1 | prostate androgen-regulated mucin-like protein 1 | 169116 | 174808 | 163743 | 43, 28, 1 |
| 4 | PRSS12 | protease, serine 12 | 164099 | 164100 | 145388 | 13, 24, 28 |
| 4 | PRSS48 | protease, serine 48 | 189099 | 109686 | 164142 | 16, |
| 4 | PRMT9 | protein arginine methyltransferase 9 | 164169 | 164168 | 071205 | |
| 4 | PRKG2 | protein kinase, cGMP-dependent, type II | 138669 | 152785 | 138670 | 24, 25, 44 |
| 4 | PPP2R2C | protein phosphatase 2 regulatory subunit Bgamma | 074211 | 109501 | 013288 | 8, 26, 38 |
| 4 | PPP3CA | protein phosphatase 3 catalytic subunit alpha | 138814 | 164035 | 153064 | 6, 26, 33 |
| 4 | PPEF2 | protein phosphatase with EF-hand domain 2 | 156194 | 138768 | 138744 | |
| 4 | PPM1K | protein phosphatase, Mg2+/Mn2+ dependent 1K | 163644 | 118777 | 138642 | 1, 29, 22 |
| 4 | PTPN13 | protein tyrosine phosphatase, non-receptor type 13 | 163629 | 109339 | 145283 | 11, 31, 6 |
| 4 | PCDH10 | Protocadherin 10 | 138650 | 151470 | 254535 | 10, 8, 17 |
| 4 | PCDH18 | Protocadherin 18 | 189184 | 254535 | 151012 | |
| 4 | PCDH7 | Protocadherin 7 | 169851 | 109689 | 047365 | 11, 26, 45 |
| 4 | QRFPR | pyroglutamylated RFamide peptide receptor | 186867 | 050730 | 164111 | 24, 17, |
| 4 | PPA2 | pyrophosphatase (inorganic) 2 | 138777 | 168769 | 236699 | 4, 11, 17 |
| 4 | PDHA2 | pyruvate dehydrogenase (lipoamide) alpha 2 | 163114 | 182168 | 163116 | 38, 8, |
| 4 | QDPR | quinoid dihydropteridine reductase | 151552 | 169744 | 249581 | 8, 34, 26 |
| 4 | RAB28 | RAB28, member RAS oncogene family | 157869 | 002587 | 109705 | 26, 34, 2 |
| 4 | RAB33B | RAB33B, member RAS oncogene family | 172007 | 164134 | 145391 | 45, 26, 31 |
| 4 | RAPGEF2 | Rap guanine nucleotide exchange factor 2 | 109756 | 164123 | 168843 | 43, 29, 31 |
| 4 | RAP1GDS1 | Rap1 GTPase-GDP dissociation stimulator 1 | 138698 | 163116 | 168785 | 29, 31, 10 |
| 4 | RASSF6 | Ras association domain family member 6 | 169435 | 079557 | 169429 | 22, 4, 42 |
| 4 | RHOH | ras homolog family member H | 168421 | 078177 | 174343 | 39, 36, 22 |
| 4 | RASL11B | RAS like family 11 member B | 128045 | 226887 | 184178 | 32, 27, 25 |
| 4 | RASGEF1B | RasGEF domain family member 1B | 138670 | 138669 | 138668 | |
| 4 | REST | RE1 silencing transcription factor | 084093 | 128040 | 084092 | 26, 29, 45 |
| 4 | RBPJ | recombination signal binding protein for immunoglobulin kappa J | 168214 | 250317 | 163394 | 1, 30, 31 |
| | | region | | | | |
| 4 | RGS12 | regulator of G-protein signaling 12 | 159788 | 188981 | 109758 | 20, 14, 17 |
| 4 | RXFP1 | relaxin/insulin like family peptide receptor 1 | 171509 | 164124 | 205208 | 25, 8, 44 |
| 4 | RELL1 | RELT like 1 | 181826 | 154274 | 169299 | 11, 40, 26 |
| 4 | RFC1 | replication factor C subunit 1 | 035928 | 157796 | 134962 | 22, 40, 4 |
| 4 | RRH | retinal pigment epithelium-derived rhodopsin homolog | 180245 | 109534 | 183423 | 10, 14, 8 |
| 4 | ARHGAP10 | Rho GTPase activating protein 10 | 071205 | 164169 | 151623 | 1, 6, 26 |
| 4 | ARHGAP24 | Rho GTPase activating protein 24 | 138639 | 163625 | 109339 | 45, 1, 43 |
| 4 | ARHGEF38 | Rho guanine nucleotide exchange factor 38 | 236699 | 138777 | 138785 | |
| 4 | RPL34 | ribosomal protein L34 | 109475 | 138795 | 198856 | 41, 12, 43 |
| 4 | RPL9 | ribosomal protein L9 | 163682 | 134962 | 121897 | |
| 4 | RPS3A | ribosomal protein S3A | 145425 | 181541 | 109686 | 41, 21, 43 |
| 4 | RCHY1 | ring finger and CHY zinc finger domain containing 1 | 163743 | 169116 | 174796 | 9, 45, 42 |
| 4 | RNF150 | ring finger protein 150 | 170153 | 109436 | 109445 | 11, 31, 9 |
| 4 | RNF175 | ring finger protein 175 | 145428 | 137462 | 145423 | 10, 14, 8 |
| 4 | RNF212 | ring finger protein 212 | 178222 | 127418 | 159674 | 40, 9, 38 |
| 4 | RNF4 | ring finger protein 4 | 063978 | 206113 | 125386 | 39, 38, 30 |
| 4 | RBM46 | RNA binding motif protein 46 | 151962 | 121207 | 185149 | 38, |
| 4 | RBM47 | RNA binding motif protein 47 | 163694 | 174343 | 179299 | 29, 42, 17 |
| 4 | POLR2B | RNA polymerase II subunit B | 047315 | 084092 | 163453 | 42, 39, 44 |
| 4 | RUFY3 | RUN and FYVE domain containing 3 | 018189 | 132467 | 132463 | 38, |
| 4 | RWDD4 | RWD domain containing 4 | 182552 | 168556 | 168538 | 31, 6, 43 |
| 4 | SWOP | S100 calcium binding protein P | 163993 | 179010 | 178988 | 37, 3, 32 |
| 4 | SGCB | sarcoglycan beta | 163069 | 188993 | 163071 | 24, 6, 45 |
| 4 | SCARB2 | scavenger receptor class B member 2 | 138760 | 138750 | 189157 | 26, 42, 13 |
| 4 | SDAD1 | SDA1 domain containing 1 | 198301 | 138744 | 279909 | 30, 21, 26 |
| 4 | SCFD2 | sec1 family domain containing 2 | 184178 | 128045 | 145216 | |
| 4 | SEC24B | SEC24 homolog B, COPII coat complex component | 138802 | 188517 | 005059 | 43, 1, 31 |
| 4 | SEC24D | SEC24 homolog D, COPII coat complex component | 150961 | 145388 | 172403 | 44, 34, 11 |
| 4 | SEC31A | SEC31 homolog A, COPII coat complex component | 138674 | 145284 | 168152 | 42, 11, 24 |
| 4 | SFRP2 | secreted frizzled related protein 2 | 145423 | 145428 | 197410 | 42, 45, 13 |
| 4 | SPP1 | secreted phosphoprotein 1 | 118785 | 152595 | 118762 | 11, 17, 32 |
| 4 | SEL1L3 | SEL1L family member 3 | 091490 | 157765 | 250317 | 22, 16, 33 |
| 4 | SEPSECS | Sep (O-phosphoserine) tRNA: Sec (selenocysteine) tRNA synthase | 109618 | 153012 | 281028 | 31, 22, 42 |
| 4 | SEPT11 | septin 11 | 138758 | 186212 | 118816 | 1, 45, 15 |
| 4 | SPINK2 | serine peptidase inhibitor, Kazal type 2 | 128040 | 171476 | 084093 | 38, 10, 5 |
| 4 | STK32B | serine/threonine kinase 32B | 152953 | 170891 | 173040 | 39, 26, 17 |
| 4 | SETD7 | SET domain containing lysine methyltransferase 7 | 145391 | 172007 | 085871 | 1, 26, 22 |
| 4 | SH3TC1 | SH3 domain and tetratricopeptide repeats 1 | 125089 | 163995 | 281359 | |
| 4 | SH3BP2 | SH3 domain binding protein 2 | 087266 | 168884 | 087274 | 36, 7, 26 |
| 4 | SH3DW | SH3 domain containing 19 | 109686 | 145425 | 189099 | |
| 4 | SH3RF1 | SH3 domain containing ring finger 1 | 154447 | 145439 | 137601 | 9, 24, 27 |
| 4 | SHISA3 | shisa family member 3 | 178343 | 188848 | 124406 | 2, 33, 45 |
| 4 | SCOC | short coiled-coil protein | 153130 | 196782 | 153132 | 29, 4, 26 |
| 4 | SHROOM3 | shroom family member 3 | 138771 | 163749 | 186212 | 13, 4, 16 |
| 4 | SPCS3 | signal peptidase complex subunit 3 | 129128 | 164122 | 150630 | 42, 43, 32 |
| 4 | SRP72 | signal recognition particle 72 | 174780 | 128050 | 196503 | 7, 45, 13 |
| 4 | STAP1 | signal transducing adaptor family member 1 | 035720 | 145241 | 033178 | 41, 21, 22 |
| 4 | SAP30 | Sin3A associated protein 30 | 164105 | 164104 | 164106 | 20, 44, 37 |
| 4 | SLAIN2 | SLAIN motif family member 2 | 109171 | 135605 | 145248 | 11, 38, 45 |
| 4 | SLIT2 | slit guidance ligand 2 | 145147 | 178177 | 163138 | 11, 31, 42 |
| 4 | SMAD1 | SMAD family member 1 | 170365 | 164164 | 151611 | 43, 1, 26 |
| 4 | SMIM14 | small integral membrane protein 14 | 163683 | 109814 | 078140 | 11, 30, 4 |
| 4 | SMIM20 | small integral membrane protein 20 | 250317 | 091490 | 168214 | 29, 22, 10 |
| 4 | SCLT1 | sodium channel and clathrin linker 1 | 151466 | 077684 | 151470 | 11, 10, 4 |
| 4 | SLC10A4 | solute carrier family 10 member 4 | 145248 | 109171 | 182223 | 14, 8, 17 |
| 4 | SLC10A6 | solute carrier family 10 member 6 | 145283 | 163629 | 163633 | 30, |
| 4 | SLC10A7 | solute carrier family 10 member 7 | 120519 | 250673 | 151615 | 42, 24, 13 |
| 4 | SLC2A9 | solute carrier family 2 member 9 | 109667 | 186146 | 169676 | 17, 27, 42 |
| 4 | SLC25A31 | solute carrier family 25 member 31 | 151475 | 164066 | 164070 | 38, 10, 7 |
| 4 | SLC25A4 | solute carrier family 25 member 4 | 151729 | 187821 | 164323 | 29, 15, 31 |
| 4 | SLC26A1 | solute carrier family 26 member 1 | 145217 | 145214 | 127415 | |
| 4 | SLC30A9 | solute carrier family 30 member 9 | 014824 | 182308 | 188848 | 18, 42, 12 |
| 4 | SLC34A2 | solute carrier family 34 member 2 | 157765 | 053900 | 091490 | 40, 27, 42 |
| 4 | SLC39A8 | solute carrier family 39 member 8 | 138821 | 153064 | 109320 | 29, 32, 16 |
| 4 | SLC4A4 | solute carrier family 4 member 4 | 080493 | 156136 | 145321 | 17, 33, 37 |
| 4 | SLC7A11 | solute carrier family 7 member 11 | 151012 | 189184 | 151014 | 37, 13, 11 |
| 4 | SLC9B1 | solute carrier family 9 member B1 | 164037 | 145354 | 164038 | 38, 28, 39 |
| 4 | SLC9B2 | solute carrier family 9 member B2 | 164038 | 164037 | 164039 | 8, |
| 4 | SORBS2 | sorbin and SH3 domain containing 2 | 154556 | 154553 | 164342 | 38, |
| 4 | SORCS2 | sortilin related VPS10 domain containing receptor 2 | 184985 | 109519 | 178597 | 14, 4, 6 |
| 4 | SNX25 | sorting nexin 25 | 109762 | 164323 | 109771 | 22, 40, 11 |
| 4 | SOWAHB | sosondowah ankyrin repeat domain family member B | 186212 | 138771 | 138758 | 17, 16, 25 |
| 4 | SPARCL1 | SPARC like 1 | 152583 | 170502 | 152591 | |
| 4 | SPOCK3 | SPARC/osteonectin, cwcv and kazal like domains proteoglycan 3 | 196104 | 038295 | 109511 | 8, 29, 17 |
| 4 | STPG2 | sperm tail PG-rich repeat containing 2 | 163116 | 163114 | 138698 | 38, 28, |
| 4 | SPATA18 | spermatogenesis associated 18 | 163071 | 163069 | 109189 | 42, 38, 29 |
| 4 | SPATA4 | spermatogenesis associated 4 | 150628 | 150627 | 164122 | 38, 30, 20 |
| 4 | SPATA5 | spermatogenesis associated 5 | 145375 | 170917 | 164056 | 6, 1, 26 |
| 4 | SGMS2 | sphingomyelin synthase 2 | 164023 | 138801 | 155016 | 1, 40, 25 |
| 4 | SPON2 | spondin 2 | 159674 | 178222 | 159692 | 33, 26, 34 |
| 4 | SPRY1 | sprouty RTK signaling antagonist 1 | 164056 | 145375 | 151458 | 4, 26, 28 |
| 4 | STBD1 | starch binding domain 1 | 118804 | 272414 | 163749 | 43, 1, 15 |
| 4 | STATH | statherin | 126549 | 135222 | 279857 | 34, 42, 25 |
| 4 | SCD5 | stearoyl-CoA desaturase 5 | 145284 | 249242 | 138674 | 11, 26, 8 |
| 4 | SLBP | stem-loop binding protein | 163950 | 174137 | 168936 | 32, 3, 38 |
| 4 | SRD5A3 | steroid 5 alpha-reductase 3 | 128039 | 128052 | 134851 | 23, 10, 27 |
| 4 | SCRG1 | stimulator of chondrogenesis 1 | 164106 | 164105 | 164107 | 45, 6, 10 |
| 4 | STOX2 | storkhead box 2 | 173320 | 168538 | 164303 | |
| 4 | STIM2 | stromal interaction molecule 2 | 109689 | 109680 | 169851 | 22, 40, 27 |
| 4 | SMR3A | submaxillary gland androgen regulated protein 3A | 109208 | 145309 | 171201 | 14, 42, |
| 4 | SMR3B | submaxillary gland androgen regulated protein 3B | 171201 | 109208 | 171199 | 25, |
| 4 | SULT1B1 | sulfotransferase family 1B member 1 | 173597 | 271271 | 109193 | 7, 45, 17 |
| 4 | SULT1E1 | sulfotransferase family 1E member 1 | 109193 | 173597 | 126545 | 2, 18, 15 |
| 4 | SOD3 | superoxide dismutase 3 | 109610 | 109606 | 181982 | 1, 26, 10 |
| 4 | SMARCA5 | SWI/SNF related, matrix associated, actin dependent | 153147 | 109458 | 183090 | 1, 11, 22 |
| | | regulator of chromatin, subfamily a, member 5 | | | | |
| 4 | SMARCAD1 | SWI/SNF-related, matrix-associated actin-dependent | 163104 | 172238 | 163106 | 43, 11, 1 |
| | | regulator of chromatin, subfamily a, containing DEAD/H box 1 | | | | |
| 4 | SYNPO2 | synaptopodin 2 | 172403 | 150961 | 172399 | 25, 44, 33 |
| 4 | STX18 | syntaxin 18 | 168818 | 168824 | 163132 | 6, 44, 25 |
| 4 | SNCA | synuclein alpha | 145335 | 185477 | 138722 | 10, 15, 26 |
| 4 | TACR3 | tachykinin receptor 3 | 169836 | 138778 | 168772 | 9, 25, |
| 4 | TBCK | TBC1 domain containing kinase | 145348 | 168743 | 164022 | 42, 21, 38 |
| 4 | TBC1D1 | TBC1 domain family member 1 | 065882 | 279386 | 250254 | 22, 21, 34 |
| 4 | TBC1D14 | TBC1 domain family member 14 | 132405 | 170871 | 173013 | 29, 22, 33 |
| 4 | TBC1D19 | TBC1 domain family member 19 | 109680 | 163394 | 109689 | 39, 29, 4 |
| 4 | TBC1D9 | TBC1 domain family member 9 | 109436 | 109424 | 170153 | 23, 1, 28 |
| 4 | TEC | tec protein tyrosine kinase | 135605 | 074966 | 109171 | 7, 2, 22 |
| 4 | TENM3 | teneurin transmembrane protein 3 | 218336 | 038002 | 129187 | 12, 11, 15 |
| 4 | TET2 | tet methylcytosine dioxygenase 2 | 168769 | 168772 | 138777 | |
| 4 | TSPAN5 | tetraspanin 5 | 168785 | 138698 | 151247 | 35, 26, 6 |
| 4 | TTC29 | tetratricopeptide repeat domain 29 | 137473 | 151615 | 151617 | 10, 38, 30 |
| 4 | THAP6 | THAP domain containing 6 | 174796 | 163743 | 174792 | 4, 26, 24 |
| 4 | THAP9 | THAP domain containing 9 | 168152 | 138674 | 189308 | 11, 42, 38 |
| 4 | THEGL | theg spermatid protein-like | 249693 | 196503 | 171476 | 38, |
| 4 | TIGD2 | tigger transposable element derived 2 | 180346 | 138640 | 185477 | 1, 34, 16 |
| 4 | TIGD4 | tigger transposable element derived 4 | 169989 | 170006 | 164144 | 38, 25, 20 |
| 4 | TNIP2 | TNFAIP3 interacting protein 2 | 168884 | 125386 | 087266 | 9, 37, 21 |
| 4 | TNIP3 | TNFAIP3 interacting protein 3 | 050730 | 173376 | 186867 | 45, 10, 9 |
| 4 | TLR1 | toll like receptor 1 | 174125 | 174123 | 174130 | 5, 22, 42 |
| 4 | TLR10 | toll like receptor 10 | 174123 | 109787 | 174125 | |
| 4 | TLR2 | toll like receptor 2 | 137462 | 121210 | 145428 | 5, 4, 20 |
| 4 | TLR3 | toll like receptor 3 | 164342 | 154556 | 109794 | 42, 32, 1 |
| 4 | TLR6 | toll like receptor 6 | 174130 | 174125 | 197712 | 22, 5, 39 |
| 4 | TLL1 | tolloid like 1 | 038295 | 109472 | 196104 | 33, 32, 12 |
| 4 | TIFA | TRAF interacting protein with forkhead associated domain | 145365 | 138660 | 073331 | 22, 41, 42 |
| 4 | TRAPPC11 | trafficking protein particle complex 11 | 168538 | 182552 | 173320 | |
| 4 | TECRL | trans-2,3-enoyl-CoA reductase-like | 205678 | 150471 | 145242 | 15, 25, 20 |
| 4 | TADA2B | transcriptional adaptor 2B | 173011 | 173013 | 109519 | 29, 22, 26 |
| 4 | TACC3 | transforming acidic coiled-coil containing protein 3 | 013810 | 168936 | 068078 | 21, 5, 22 |
| 4 | TRPC3 | transient receptor potential cation channel subfamily C | 138741 | 138686 | 138688 | 15, 8, |
| | | member 3 | | | | |
| 4 | TKTL2 | transketolase like 2 | 151005 | 164129 | 198498 | 38, 24, 8 |
| 4 | TMA16 | translation machinery associated 16 homolog | 198498 | 151005 | 145416 | 34, 7, 6 |
| 4 | TRAM1L1 | translocation associated membrane protein 1-like 1 | 174599 | 270394 | 164100 | 11, 38, 8 |
| 4 | TAPT1 | transmembrane anterior posterior transformation 1 | 169762 | 007062 | 169744 | 26, 22, 10 |
| 4 | TMPRSS11A | transmembrane protease, serine 11A | 187054 | 153802 | 198092 | 24, 13, 42 |
| 4 | TMPRSS11B | transmembrane protease, serine 11B | 185873 | 198092 | 083896 | 13, 18, 30 |
| 4 | TMPRSS11D | transmembrane protease, serine 11D | 153802 | 109163 | 187054 | 24, 30, 13 |
| 4 | TMPRSS11E | transmembrane protease, serine 11E | 087128 | 083896 | 197888 | 13, 30, 24 |
| 4 | TMPRSS11F | transmembrane protease, serine 11F | 198092 | 187054 | 185873 | 13, 24, 32 |
| 4 | TMEM128 | transmembrane protein 128 | 132406 | 163982 | 145220 | 34, 2, 43 |
| 4 | TMEM129 | transmembrane protein 129 | 168936 | 163950 | 013810 | 1, 34, 26 |
| 4 | TMEM144 | transmembrane protein 144 | 164124 | 164125 | 171509 | 8, 38, 6 |
| 4 | TMEM150C | transmembrane protein 150C | 249242 | 145293 | 145284 | 36, 8, 24 |
| 4 | TMEM154 | transmembrane protein 154 | 170006 | 109670 | 169989 | 24, 5, 45 |
| 4 | TMEM155 | transmembrane protein 155 | 164112 | 164111 | 123737 | 32, 39, 8 |
| 4 | TMEM156 | transmembrane protein 156 | 121895 | 197712 | 109790 | 7, 45, 34 |
| 4 | TMEM165 | transmembrane protein 165 | 134851 | 128039 | 134852 | 22, 45, 6 |
| 4 | TMEM175 | transmembrane protein 175 | 127419 | 178950 | 145214 | 5, 26, 27 |
| 4 | TMEM184C | transmembrane protein 184C | 164168 | 151617 | 164169 | 40, 29, 30 |
| 4 | TMEM192 | transmembrane protein 192 | 170088 | 176979 | 109466 | 31, 17, 40 |
| 4 | TMEM33 | transmembrane protein 33 | 109133 | 109132 | 182308 | 13, 22, 42 |
| 4 | TRIM2 | tripartite motif containing 2 | 109654 | 137460 | 121211 | 8, 26, 31 |
| 4 | TRIM60 | tripartite motif containing 60 | 176979 | 250486 | 170088 | 38, |
| 4 | TRIM61 | tripartite motif containing 61 | 183439 | 248329 | 250486 | 38, 6, 28 |
| 4 | TRIML1 | tripartite motif family like 1 | 184108 | 179046 | 109536 | 2, 38, 32 |
| 4 | TRIML2 | tripartite motif family like 2 | 179046 | 179059 | 184108 | |
| 4 | TRMT10A | tRNA methyltransferase 10A | 145331 | 138813 | 138823 | 9, 22, 26 |
| 4 | TRMT44 | tRNA methyltransferase 44 homolog (S. cerevisiae) | 155275 | 087008 | 155269 | 40, 27, 37 |
| 4 | TDO2 | tryptophan 2,3-dioxygenase | 151790 | 256394 | 256043 | 19, 4, 31 |
| 4 | TXK | TXK tyrosine kinase | 074966 | 198515 | 135605 | 32, 41, 39 |
| 4 | UBE2D3 | ubiquitin conjugating enzyme E2 D3 | 109332 | 109323 | 145354 | 39, 2, 4 |
| 4 | UBE2K | ubiquitin conjugating enzyme E2 K | 078140 | 163683 | 121892 | 31, 29, 4 |
| 4 | UCHL1 | ubiquitin C-terminal hydrolase L1 | 154277 | 163697 | 064042 | 26, 28, 20 |
| 4 | UBA6 | ubiquitin like modifier activating enzyme 6 | 033178 | 035720 | 109163 | 4, 26, 9 |
| 4 | USP17L10 | ubiquitin specific peptidase 17-like family member 10 | 231396 | 229924 | 233136 | |
| 4 | USP17L11 | ubiquitin specific peptidase 17-like family member 11 | 233136 | 231396 | 227551 | |
| 4 | USP17L12 | ubiquitin specific peptidase 17-like family member 12 | 227551 | 233136 | 232399 | |
| 4 | USP17L13 | ubiquitin specific peptidase 17-like family member 13 | 232399 | 227551 | 223569 | |
| 4 | USP17L15 | ubiquitin specific peptidase 17-like family member 15 | 223569 | 232399 | 249104 | 16, |
| 4 | USP17L17 | ubiquitin specific peptidase 17-like family member 17 | 249104 | 223569 | 250844 | |
| 4 | USP17L18 | ubiquitin specific peptidase 17-like family member 18 | 250844 | 249104 | 248920 | |
| 4 | USP17L19 | ubiquitin specific peptidase 17-like family member 19 | 248920 | 250844 | 250745 | |
| 4 | USP17L20 | ubiquitin specific peptidase 17-like family member 20 | 250745 | 248920 | 249811 | |
| 4 | USP17L21 | ubiquitin specific peptidase 17-like family member 21 | 249811 | 250745 | 248933 | |
| 4 | USP17L22 | ubiquitin specific peptidase 17-like family member 22 | 248933 | 249811 | 250913 | |
| 4 | USP17L23 | ubiquitin specific peptidase 17-like family member 23 | 250913 | 248933 | 232264 | |
| 4 | USP17L24 | ubiquitin specific peptidase 17-like family member 24 | 232264 | 250913 | 230430 | |
| 4 | USP17L25 | ubiquitin specific peptidase 17-like family member 25 | 230430 | 232264 | 229579 | |
| 4 | USP17L26 | ubiquitin specific peptidase 17-like family member 26 | 229579 | 230430 | 227140 | |
| 4 | USP17L27 | ubiquitin specific peptidase 17-like family member 27 | 235780 | 227140 | 231051 | |
| 4 | USP17L28 | ubiquitin specific peptidase 17-like family member 28 | 231051 | 235780 | 231637 | |
| 4 | USP17L29 | ubiquitin specific peptidase 17-like family member 29 | 231637 | 231051 | 228856 | |
| 4 | USP17L30 | ubiquitin specific peptidase 17-like family member 30 | 228856 | 231637 | 186146 | |
| 4 | USP17L5 | ubiquitin specific peptidase 17-like family member 5 | 227140 | 229579 | 235780 | |
| 4 | USP38 | ubiquitin specific peptidase 38 | 170185 | 109452 | 109458 | 22, 11, 2 |
| 4 | USP46 | ubiquitin specific peptidase 46 | 109189 | 163071 | 226887 | 8, 37, 29 |
| 4 | USP53 | ubiquitin specific peptidase 53 | 145390 | 172399 | 164096 | 1, 45, 29 |
| 4 | UGT2A1 | UDP glucuronosyltransferase family 2 member A1 complex locus | 173610 | 156096 | 271271 | 42, 8, |
| 4 | UGT2A2 | UDP glucuronosyltransferase family 2 member A2 | 271271 | 173610 | 173597 | |
| 4 | UGT2A3 | UDP glucuronosyltransferase family 2 member A3 | 135220 | 109181 | 171234 | 17, 16, 4 |
| 4 | UGT2B10 | UDP glucuronosyltransferase family 2 member B10 | 109181 | 196620 | 135220 | 19, 10, 23 |
| 4 | UGT2B11 | UDP glucuronosyltransferase family 2 member B11 | 213759 | 171234 | 135226 | 10, 25, 33 |
| 4 | UGT2B15 | UDP glucuronosyltransferase family 2 member B15 | 196620 | 197888 | 109181 | 4, 19, 23 |
| 4 | UGT2B17 | UDP glucuronosyltransferase family 2 member B17 | 197888 | 087128 | 196620 | 10, 16, 33 |
| 4 | UGT2B28 | UDP glucuronosyltransferase family 2 member B28 | 135226 | 213759 | 156096 | 10, 33, |
| 4 | UGT2B4 | UDP glucuronosyltransferase family 2 member B4 | 156096 | 135226 | 173610 | 19, 15, 10 |
| 4 | UGT2B7 | UDP glucuronosyltransferase family 2 member B7 | 171234 | 135220 | 213759 | 17, 10, 19 |
| 4 | UGT8 | UDP glycosyltransferase 8 | 174607 | 180801 | 138653 | 11, 8, 24 |
| 4 | UGDH | UDP-glucose 6-dehydrogenase | 109814 | 121897 | 163683 | 13, 45, 4 |
| 4 | UFSP2 | UFM1 specific peptidase 2 | 109775 | 186352 | 205129 | 24, 45, 5 |
| 4 | UNC5C | unc-5 netrin receptor C | 182168 | 138696 | 163114 | 11, 23, 40 |
| 4 | UCP1 | uncoupling protein 1 | 109424 | 179387 | 109436 | 1, 10, |
| 4 | USO1 | USO1 vesicle transport factor | 138768 | 138757 | 156194 | 42, 45, 31 |
| 4 | UTP3 | UTP3, small subunit processome component homolog | 132467 | 132465 | 018189 | 13, 37, 36 |
| | | (S. cerevisiae) | | | | |
| 4 | UVSSA | UV stimulated scaffold protein A | 163945 | 090316 | 179979 | 39, 31, 40 |
| 4 | VEGFC | vascular endothelial growth factor C | 150630 | 129128 | 109674 | 35, 6, 28 |
| 4 | WDFY3 | WD repeat and FYVE domain containing 3 | 163625 | 163624 | 138639 | 18, 40, 26 |
| 4 | WDR1 | WD repeat domain 1 | 071127 | 169676 | 178163 | 45, 39, 17 |
| 4 | WDR17 | WD repeat domain 17 | 150627 | 150625 | 150628 | |
| 4 | WDR19 | WD repeat domain 19 | 157796 | 109790 | 035928 | 31, 26, 28 |
| 4 | WHSC1 | Wolf-Hirschhorn syndrome candidate 1 | 109685 | 168924 | 185049 | 22, 12, 31 |
| 4 | WFS1 | wolframin ER transmembrane glycoprotein | 109501 | 152969 | 074211 | 33, 38, 20 |
| 4 | WWC2 | WW and C2 domain containing 2 | 151718 | 129187 | 177300 | 45, 19, 12 |
| 4 | YIPF7 | Yip1 domain family member 7 | 177752 | 183783 | 151806 | |
| 4 | YTHDC1 | YTH domain containing 1 | 083896 | 185873 | 087128 | 4, 44, 26 |
| 4 | ZFP42 | ZFP42 zinc finger protein | 179059 | 083857 | 179046 | 32, 12, |
| 4 | ZBTB49 | zinc finger and BTB domain containing 49 | 168826 | 145220 | 168824 | 13, 40, 39 |
| 4 | ZCCHC4 | zinc finger CCHC-type containing 4 | 168228 | 038210 | 053900 | 34, 1, 24 |
| 4 | ZFYVE28 | zinc finger FYVE-type containing 28 | 159733 | 123933 | 206113 | 1, 39, 28 |
| 4 | ZGRF1 | zinc finger GRF-type containing 1 | 138658 | 178403 | 174720 | 11, 20, 22 |
| 4 | ZNF141 | zinc finger protein 141 | 131127 | 186777 | 182903 | |
| 4 | ZNF330 | zinc finger protein 330 | 109445 | 170153 | 164136 | 42, 4, 17 |
| 4 | ZNF518B | zinc finger protein 518B | 178163 | 071127 | 109684 | 11, 26, 31 |
| 4 | ZNF595 | zinc finger protein 595 | 272602 | 151725 | 250312 | 34, 1, 7 |
| 4 | ZNF718 | zinc finger protein 718 | 250312 | 272602 | 186777 | 45, 3, 19 |
| 4 | ZNF721 | zinc finger protein 721 | 182903 | 131127 | 174227 | |
| 4 | ZNF732 | zinc finger protein 732 | 186777 | 250312 | 131127 | 39, 12, 38 |
| 4 | ZNF827 | zinc finger protein 827 | 151612 | 237136 | 164167 | 35, |
| 4 | ZAR1 | zygote arrest 1 | 182223 | 145248 | 075539 | 23, 38, |
| 5 | HMGCR | 3-hydroxy-3-methylglutaryl-CoA reductase | 113161 | 145700 | 113163 | 42, 24, 8 |
| 5 | HMGCS1 | 3-hydroxy-3-methylglutaryl-CoA synthase 1 | 112972 | 177453 | 151882 | |
| 5 | OXCT1 | 3-oxoacid CoA-transferase 1 | 083720 | 182836 | 205765 | 42, 39, 3 |
| 5 | HTR1A | 5-hydroxytryptamine receptor 1A | 178394 | 186105 | 164197 | 8, |
| 5 | HTR4 | 5-hydroxytryptamine receptor 4 | 164270 | 145868 | 169252 | 26, 2, 37 |
| 5 | MTRR | 5-methyltetrahydrofolate-homocysteine methyltransferase | 124275 | 215217 | 124279 | 29, 42, 9 |
| | | reductase | | | | |
| 5 | PHYKPL | 5-phosphohydroxy-L-lysine phospho-lyase | 175309 | 197451 | 050767 | 30, 36, 18 |
| 5 | ABLIM3 | actin binding LIM protein family member 3 | 173210 | 169247 | 157510 | 34, 1, 40 |
| 5 | AFAP1L1 | actin filament associated protein 1 like 1 | 157510 | 173210 | 164284 | 34, 32, 45 |
| 5 | ACTBL2 | actin, beta like 2 | 169067 | 062194 | 145632 | 17, |
| 5 | ACSL6 | acyl-CoA synthetase long-chain family member 6 | 164398 | 281938 | 164399 | 38, 14, 8 |
| 5 | ACOT12 | acyl-CoA thioesterase 12 | 172497 | 131732 | 145687 | 19, 10, 20 |
| 5 | ADAM19 | ADAM metallopeptidase domain 19 | 135074 | 172568 | 172548 | 6, 35, 39 |
| 5 | ADAMTS12 | ADAM metallopeptidase with thrombospondin type 1 motif 12 | 151388 | 113407 | 182631 | 15, 43, 6 |
| 5 | ADAMTS16 | ADAM metallopeptidase with thrombospondin type 1 motif 16 | 145536 | 170549 | 164151 | 22, 27, 6 |
| 5 | ADAMTS19 | ADAM metallopeptidase with thrombospondin type 1 motif 19 | 145808 | 066583 | 186367 | 23, 12, 44 |
| 5 | ADAMTS2 | ADAM metallopeptidase with thrombospondin type 1 motif 2 | 087116 | 177932 | 176783 | 18, 6, 7 |
| 5 | ADAMTS6 | ADAM metallopeptidase with thrombospondin type 1 motif 6 | 049192 | 153015 | 123219 | 32, 19, 15 |
| 5 | AP3B1 | adapter related protein complex 3 beta 1 subunit | 132842 | 171530 | 085365 | 40, 22, 31 |
| 5 | AP3S1 | adapter related protein complex 3 sigma 1 subunit | 177879 | 145782 | 280329 | 11, 43, 4 |
| 5 | ADCY2 | adenylate cyclase 2 | 078295 | 112941 | 215217 | 26, 8, 42 |
| 5 | AK6 | adenylate kinase 6 | 085231 | 183323 | 273841 | |
| 5 | ADGRV1 | adhesion G protein-coupled receptor V1 | 164199 | 176018 | 113369 | 10, 2, 11 |
| 5 | ARL10 | ADP ribosylation factor like GTPase 10 | 175414 | 122203 | 048162 | 14, 44, 16 |
| 5 | ARL14EPL | ADP ribosylation factor like GTPase 14 effector protein like | 268223 | 268714 | 145781 | 32, |
| 5 | ARL15 | ADP ribosylation factor like GTPase 15 | 185305 | 164258 | 169271 | 4, 34, 15 |
| 5 | ADRA1B | adrenoceptor alpha 1B | 170214 | 113302 | 113312 | 45, 17, 6 |
| 5 | ADRB2 | adrenoceptor beta 2 | 169252 | 164270 | 169247 | 36, 42, 40 |
| 5 | AFF4 | AF4/FMR2 family member 4 | 072364 | 164406 | 155329 | 23, 39, 44 |
| 5 | AGXT2 | alanine--glyoxylate aminetransferase 2 | 113492 | 168724 | 113494 | 17, 19, 12 |
| 5 | ALDH7A1 | aldehyde dehydrogenase 7 family member A1 | 164904 | 155324 | 164902 | 18, 32, 11 |
| 5 | AMACR | alpha-methylacyl-CoA racemase | 242110 | 164175 | 273294 | 33, 17, 9 |
| 5 | APBB3 | amyloid beta precursor protein binding family B member 3 | 113108 | 243056 | 176087 | 26, 39, 40 |
| 5 | AGGF1 | angiogenic factor with G-patch and FHA domains 1 | 164252 | 145708 | 132846 | 30, 6, 22 |
| 5 | ANKH | ANKH inorganic pyrophosphate transport regulator | 154122 | 154124 | 183580 | |
| 5 | ANKHD1- | ANKHD1-EIF4EBP3 readthrough | 254996 | 131503 | 213523 | 18, 22, 11 |
| | EIF4EBP3 | | | | | |
| 5 | ANKDD1B | ankyrin repeat and death domain containing 1B | 189045 | 122008 | 152359 | 44, 20, 17 |
| 5 | ANKHD1 | ankyrin repeat and KH domain containing 1 | 131503 | 113073 | 254996 | 18, 22, 11 |
| 5 | ANKRD31 | ankyrin repeat domain 31 | 145700 | 176928 | 113161 | 10, 15, 38 |
| 5 | ANKRD33B | ankyrin repeat domain 33B | 164236 | 145491 | 112977 | 36, 14, 15 |
| 5 | ANKRD34B | ankyrin repeat domain 34B | 189127 | 152380 | 228716 | 22, 8, 17 |
| 5 | ANKRD55 | ankyrin repeat domain 55 | 164512 | 134352 | 225940 | 10, 30, 38 |
| 5 | ANKRA2 | ankyrin repeat family A member 2 | 164331 | 145741 | 164338 | 31, 9, 11 |
| 5 | ANXA2R | annexin A2 receptor | 177721 | 250722 | 172262 | 22, 34, 3 |
| 5 | ANXA6 | annexin A6 | 197043 | 145901 | 198624 | 7, 36, 26 |
| 5 | ATOX1 | antioxidant 1 capper chaperone | 177556 | 113140 | 145907 | 31, 19, 7 |
| 5 | APC | APC, WNT signaling pathway regulator | 134982 | 129595 | 258864 | 26, 8, 11 |
| 5 | ARAP3 | ArfGAP with RhoGAP domain, ankyrin repeat and PH domain 3 | 120318 | 197948 | 156453 | 25, 14, 7 |
| 5 | RARS | arginyl-tRNA synthetase | 113643 | 113645 | 188573 | 45, 3, 9 |
| 5 | ARRDC3 | arrestin domain containing 3 | 113369 | 164199 | 175745 | 42, 29, 4 |
| 5 | AHRR | aryl-hydrocarbon receptor repressor | 063438 | 249915 | 180104 | |
| 5 | ARSB | arylsulfatase B | 113273 | 145685 | 132837 | 10, 7, 36 |
| 5 | ARSI | arylsulfatase family member I | 183876 | 070808 | 070814 | 14, 24, 32 |
| 5 | ARSK | arylsulfatase family member K | 164291 | 198677 | 178015 | 29, 45, 42 |
| 5 | ATP6AP1L | ATPase H+ transporting accessory protein 1 like | 205464 | 186468 | 174695 | 34, 33, 28 |
| 5 | ATP6V0E1 | ATPase H+ transporting V0 subunit e1 | 113732 | 037241 | 164463 | 27, 43, 31 |
| 5 | ATP10B | ATPase phospholipid transporting 10B (putative) | 118322 | 164611 | 145864 | 16, 8, 14 |
| 5 | ATG10 | autophagy related 10 | 152348 | 145687 | 186468 | 33, 30, 29 |
| 5 | ATG12 | autophagy related 12 | 145782 | 129596 | 177879 | 4, 18, 7 |
| 5 | BDP1 | B double prime 1, subunit of RNA polymerase III | 145734 | 145736 | 131844 | 11, 5, 4 |
| | | transcription initiation factor IIIB | | | | |
| 5 | BTF3 | basic transcription factor 3 | 145741 | 251493 | 164331 | 4, 30, 28 |
| 5 | BNIP1 | BCL2 interacting protein 1 | 113734 | 164463 | 183072 | 29, 9, 21 |
| 5 | B4GALT7 | beta-1,4-galactosyltransferase 7 | 027847 | 184840 | 170074 | 29, 27, 1 |
| 5 | BHMT | betaine--homocysteine S-methyltransferase | 145692 | 132840 | 152409 | 17, 19, 14 |
| 5 | BHMT2 | betaine--homocysteine S-methyltransferase 2 | 132840 | 132837 | 145692 | 17, 19, 1 |
| 5 | BOD1 | biorientation of chromosomes in cell division 1 | 145919 | 113739 | 113742 | 31, 21, 9 |
| 5 | BASP1 | brain abundant membrane attached signal protein 1 | 176788 | 145555 | 145526 | 22, 41, 45 |
| 5 | BRD8 | bromodomain containing 8 | 112983 | 112981 | 112984 | 31, 11, 39 |
| 5 | BRD9 | bromodomain containing 9 | 028310 | 188818 | 071539 | 22, 40, 18 |
| 5 | BRIX1 | BRX1, biogenesis of ribosomes | 113460 | 113456 | 168724 | 31, 34, 30 |
| 5 | BTNL3 | butyrophilin like 3 | 168903 | 113303 | 165810 | 16, |
| 5 | BTNL8 | butyrophilin like 8 | 113303 | 196670 | 168903 | 16, 5, 3 |
| 5 | BTNL9 | butyrophilin like 9 | 165810 | 168903 | 185372 | 1, 22, 36 |
| 5 | C1QTNF2 | C1q and tumor necrosis factor related protein 2 | 145861 | 135083 | 221886 | 15, 14, 10 |
| 5 | C1QTNF3 | C1q and tumor necrosis factor related protein 3 | 082196 | 273294 | 039560 | 43, 10, 11 |
| 5 | C1QTNF3- | C1QTNF3-AMACR readthrough (NMD candidate) | 273294 | 242110 | 082196 | |
| | AMACR | | | | | |
| 5 | CDH10 | cadherin 10 | 040731 | 164256 | 113100 | 2, 8, 9 |
| 5 | CDH12 | cadherin 12 | 154162 | 145526 | 164256 | 10, 42, 2 |
| 5 | CDH18 | cadherin 18 | 145526 | 176788 | 154162 | 8, 27, 23 |
| 5 | CDH6 | cadherin 6 | 113361 | 113100 | 113360 | |
| 5 | CDH9 | cadherin 9 | 113100 | 040731 | 113361 | 17, 8, |
| 5 | CDHR2 | cadherin related family member 2 | 074276 | 146083 | 169258 | 38, 16, 42 |
| 5 | CAMLG | calcium modulating ligand | 164615 | 113615 | 145833 | 10, 38, 13 |
| 5 | CAMK2A | calcium/calmodulin dependent protein kinase II alpha | 070808 | 011083 | 183876 | 8, 17, 42 |
| 5 | CAMK4 | calcium/calmodulin dependent protein kinase IV | 152495 | 134987 | 164211 | 39, 38, 8 |
| 5 | CAPSL | calcyphosine like | 152611 | 168685 | 145626 | 30, 10, 31 |
| 5 | CANX | calnexin | 127022 | 204659 | 161021 | 42, 19, 9 |
| 5 | CAST | calpastatin | 153113 | 175426 | 164307 | |
| 5 | CMBL | carboxymethylenebutenolidase homolog | 164237 | 150753 | 145495 | 17, |
| 5 | CMYA5 | cardiomyopathy associated 5 | 164309 | 164329 | 177034 | 25, 15, 26 |
| 5 | CARTPT | CART prepropeptide | 164326 | 131844 | 131711 | 31, 2, 14 |
| 5 | CSNK1A1 | casein kinase 1 alpha 1 | 113712 | 127743 | 183111 | |
| 5 | CSNK1G3 | casein kinase 1 gamma 3 | 151292 | 168944 | 168916 | 29, 2, 4 |
| 5 | CARD6 | caspase recruitment domain family member 6 | 132357 | 145592 | 112936 | 36, 34, 45 |
| 5 | CTNNA1 | catenin alpha 1 | 044115 | 113013 | 146006 | |
| 5 | CTNND2 | catenin delta 2 | 169862 | 112977 | 039139 | 8, 10, 26 |
| 5 | CATSPER3 | cation channel sperm associated 3 | 152705 | 132570 | 069011 | 38, 16, 22 |
| 5 | CDX1 | caudal type homeobox 1 | 113722 | 113721 | 011083 | 3, 16, |
| 5 | CCL28 | C-C motif chemokine ligand 28 | 151882 | 112972 | 151881 | 42, 23, 4 |
| 5 | CNOT6 | CCR4-NOT transcription complex subunit 6 | 113300 | 131459 | 161055 | 11, 12, 22 |
| 5 | CNOT8 | CCR4-NOT transcription complex subunit 8 | 155508 | 170271 | 082516 | |
| 5 | CD14 | CD14 molecule | 170458 | 176087 | 131495 | 1, 32, 27 |
| 5 | CD180 | CD180 molecule | 134061 | 069020 | 145675 | 36, 22, 41 |
| 5 | CD74 | CD74 molecule | 019582 | 070814 | 164587 | 41, 36, 21 |
| 5 | CLK4 | CDC like kinase 4 | 113240 | 050767 | 169131 | 7, 13, 43 |
| 5 | CDC42SE2 | CDC42 small effector 2 | 158985 | 186687 | 158987 | 42, 39, 38 |
| 5 | CDKN2AIPNL | CDKN2A interacting protein N-terminal like | 237190 | 119048 | 043143 | 34, 26, 21 |
| 5 | CDC20B | cell division cycle 20B | 164287 | 145649 | 164294 | 24, 42, 44 |
| 5 | CDC23 | cell division cycle 23 | 094880 | 112984 | 146013 | 4, 39, 34 |
| 5 | CDC25C | cell division cycle 25C | 158402 | 146013 | 120709 | 44, 38, 6 |
| 5 | CETN3 | centrin 3 | 153140 | 081189 | 176055 | 11, 4, 34 |
| 5 | CENPH | centromere protein H | 153044 | 134057 | 134056 | 34, 21, 12 |
| 5 | CENPK | centromere protein K | 123219 | 049192 | 113593 | 4, 7, 34 |
| 5 | CEP120 | centrosomal protein 120 | 168944 | 061455 | 151292 | 22, 1, 7 |
| 5 | CEP72 | centrosomal protein 72 | 112877 | 066230 | 171368 | 13, 28, 38 |
| 5 | CCT5 | chaperonin containing TCP1 subunit 5 | 150753 | 150756 | 164237 | 13, 9, 4 |
| 5 | CBY3 | chibby family member 3 | 204659 | 169045 | 127022 | 45, 38, 8 |
| 5 | CHSY3 | chondroitin sulfate synthase 3 | 198108 | 186367 | 169567 | 1, 27, 37 |
| 5 | CHD1 | chromodomain helicase DNA binding protein 1 | 153922 | 279232 | 174132 | 39, 25, 18 |
| 5 | C5orf15 | chromosome 5 open reading frame 15 | 113583 | 053108 | 213585 | 13, 22, 45 |
| 5 | C5orf22 | chromosome 5 open reading frame 22 | 082213 | 113360 | 133401 | 4, 42, 2 |
| 5 | C5orf24 | chromosome 5 open reading frame 24 | 181904 | 145833 | 113621 | |
| 5 | C5orf30 | chromosome 5 open reading frame 30 | 181751 | 145725 | 112874 | 43, 9, 8 |
| 5 | C5orf34 | chromosome 5 open reading frame 34 | 172244 | 151881 | 172239 | 22, 45, 37 |
| 5 | C5orf38 | chromosome 5 open reading frame 38 | 186493 | 170561 | 170549 | 6, 33, 12 |
| 5 | C5orf42 | chromosome 5 open reading frame 42 | 197603 | 164190 | 113569 | 40, 26, 7 |
| 5 | C5orf46 | chromosome 5 open reading frame 46 | 178776 | 164265 | 133710 | 15, 45, 42 |
| 5 | C5orf47 | chromosome 5 open reading frame 47 | 185056 | 113742 | 170091 | 38, 25, 19 |
| 5 | C5orf49 | chromosome 5 open reading frame 49 | 215217 | 078295 | 124275 | 14, 38, 8 |
| 5 | C5orf51 | chromosome 5 open reading frame 51 | 205765 | 083720 | 151876 | 29, 11, 22 |
| 5 | C5orf52 | chromosome 5 open reading frame 52 | 187658 | 039600 | 113272 | 38, 10, |
| 5 | C5orf56 | Chromosome 5 open reading frame 56 | 197536 | 197375 | 125347 | 7, 39, 20 |
| 5 | C5orf58 | chromosome 5 open reading frame 58 | 234511 | 168269 | 043462 | 38, 36, 6 |
| 5 | C5orf63 | chromosome 5 open reading frame 63 | 164241 | 173926 | 145794 | 29, 7, 18 |
| 5 | C5orf67 | chromosome 5 open reading frame 67 | 225940 | 164512 | 095015 | 40, 14, 15 |
| 5 | CLINT1 | clathrin interactor 1 | 113282 | 155858 | 164330 | 4, 42, 30 |
| 5 | CLTB | clathrin light chain B | 175416 | 146066 | 113194 | 19, |
| 5 | CLPTM1L | CLPTM1 like | 049656 | 164362 | 142319 | 9, 37, 16 |
| 5 | F2R | coagulation factor II thrombin receptor | 181104 | 164220 | 164251 | |
| 5 | F2RL2 | coagulation factor II thrombin receptor like 2 | 164220 | 145703 | 181104 | 42, 17, 45 |
| 5 | F12 | coagulation factor XII | 131187 | 196570 | 198055 | 3, 37, 19 |
| 5 | CCDC112 | coiled-coil domain containing 112 | 164221 | 164219 | 145780 | 24, 10, 26 |
| 5 | CCDC125 | coiled-coil domain containing 125 | 183323 | 134058 | 085231 | 1, 3, 22 |
| 5 | CCDC127 | coiled-coil domain containing 127 | 164366 | 185028 | 073578 | 9, 30, 13 |
| 5 | CCDC152 | coiled-coil domain containing 152 | 198865 | 112964 | 250722 | 34, 36, 22 |
| 5 | CCDC192 | coiled-coil domain containing 192 | 230561 | 205279 | 064651 | |
| 5 | CCDC69 | coiled-coil domain containing 69 | 198624 | 197043 | 196743 | 21, 41, 22 |
| 5 | COL4A3BP | collagen type IV alpha 3 binding protein | 113163 | 113161 | 122008 | 29, 26, 22 |
| 5 | COL23A1 | collagen type XXIII alpha 1 chain | 050767 | 175309 | 113240 | 40, 15, 17 |
| 5 | CSF1R | colony stimulating factor 1 receptor | 182578 | 113716 | 113721 | 7, 32, 26 |
| 5 | CSF2 | colony stimulating factor 2 | 164400 | 164399 | 072682 | 20, 5, 6 |
| 5 | COMMD10 | COMM domain containing 10 | 145781 | 268223 | 092421 | 10, 31, 2 |
| 5 | C6 | complement C6 | 039537 | 171495 | 182836 | 19, 4, 28 |
| 5 | C7 | complement C7 | 112936 | 132357 | 171495 | 1, 2, 42 |
| 5 | C9 | complement C9 | 113600 | 082074 | 153071 | 19, 25, 17 |
| 5 | CPLX2 | complexin 2 | 145920 | 113749 | 051596 | 8, 26, 20 |
| 5 | CTXN3 | cortexin 3 | 205279 | 164244 | 230561 | 17, 8, |
| 5 | CRHBP | corticotropin releasing hormone binding protein | 145708 | 171643 | 164252 | 2, 17, 32 |
| 5 | CKMT2 | creatine kinase, mitochondrial 2 | 131730 | 113319 | 131732 | 25, 15, 29 |
| 5 | CREBRF | CREB3 regulatory factor | 164463 | 113732 | 113734 | 22, 7, 31 |
| 5 | CWC27 | CWC27 spliceosome associated protein homolog | 153015 | 153006 | 049192 | 10, 34, 6 |
| 5 | CXCL14 | C-X-C motif chemokine ligand 14 | 145824 | 181965 | 250378 | 26, 14, 17 |
| 5 | CXXC5 | CXXC finger protein 5 | 171604 | 131508 | 146005 | 31, 14, 29 |
| 5 | CCNB1 | cyclin B1 | 134057 | 145740 | 153044 | 21, 35, 13 |
| 5 | CDK7 | cyclin dependent kinase 7 | 134058 | 134056 | 183323 | 43, 9, 23 |
| 5 | CDKL3 | cyclin dependent kinase like 3 | 006837 | 113575 | 119048 | 38, 15, 42 |
| 5 | CCNG1 | cyclin G1 | 113328 | 113327 | 170584 | 4, 45, 7 |
| 5 | CCNH | cyclin H | 134480 | 145715 | 164180 | 7, 6, 31 |
| 5 | CCNI2 | cyclin I family member 2 | 205089 | 131437 | 164402 | 20, 23, 8 |
| 5 | CCNJL | cyclin J like | 135083 | 170231 | 145861 | 38, 27, 16 |
| 5 | CCNO | cyclin O | 152669 | 234602 | 067248 | 3, 4, 9 |
| 5 | CDO1 | cysteine dioxygenase type 1 | 129596 | 134970 | 145782 | 1, 10, 32 |
| 5 | CYSTM1 | cysteine rich transmembrane module containing 1 | 120306 | 182700 | 113068 | 10, 29, 3 |
| 5 | COX7C | cytochrome c oxidase subunit 7C | 127184 | 164176 | 145715 | 15, 12, 25 |
| 5 | CYFIP2 | cytoplasmic FMR1 interacting protein 2 | 055163 | 170613 | 172568 | 21, 22, 43 |
| 5 | CPEB4 | cytoplasmic polyadenylation element binding protein 4 | 113742 | 145919 | 185056 | 25, 29, 32 |
| 5 | DAB2 | DAB2, clathrin adaptor protein | 153071 | 113600 | 113638 | 32, 11, 35 |
| 5 | DDX4 | DEAD-box helicase 4 | 152670 | 177058 | 164509 | 38, 8, |
| 5 | DDX41 | DEAD-box helicase 41 | 183258 | 146094 | 146067 | 36, 37, 39 |
| 5 | DDX46 | DEAD-box helicase 46 | 145833 | 164615 | 181904 | 18, 31, 24 |
| 5 | DAP | death associated protein | 112977 | 164236 | 169862 | 40, 4, 34 |
| 5 | DCP2 | decapping mRNA 2 | 172795 | 212643 | 171444 | 22, 21, 30 |
| 5 | DOCK2 | dedicator of cytokinesis 2 | 134516 | 040275 | 204767 | 22, 21, 39 |
| 5 | DCANP1 | dendritic cell associated nuclear protein | 251380 | 113648 | 255833 | 15, 7, 36 |
| 5 | DEPDC1B | DEP domain containing 1B | 035499 | 280447 | 164181 | 32, 3, 7 |
| 5 | DHX29 | DExH-box helicase 29 | 067248 | 152669 | 039123 | 29, 22, 43 |
| 5 | DIAPH1 | diaphanous related formin 1 | 131504 | 240764 | 171720 | 22, 40, 18 |
| 5 | DHFR | dihydrofolate reductase | 228716 | 189127 | 113318 | |
| 5 | DPYSL3 | dihydropyrimidinase like 3 | 113657 | 169302 | 176049 | 12, 1, 14 |
| 5 | DIMT1 | DIM1 dimethyladenosine transferase 1 homolog | 086189 | 068796 | 086200 | 4, 45, 33 |
| 5 | DMGDH | dimethylglycine dehydrogenase | 132837 | 113273 | 132840 | 19, 17, 42 |
| 5 | PPIP5K2 | diphosphoinositol pentakisphosphate kinase 2 | 145725 | 145723 | 181751 | 7, 45, 13 |
| 5 | DMXL1 | Dmx like 1 | 172869 | 169570 | 145779 | 22, 40, 10 |
| 5 | POLK | DNA polymerase kappa | 122008 | 113163 | 189045 | 31, 42, 45 |
| 5 | DNAJC18 | DnaJ heat shock protein family (Hsp40) member C18 | 170464 | 170469 | 279686 | 8, 38, 31 |
| 5 | DNAJC21 | DnaJ heat shock protein family (Hsp40) member C21 | 168724 | 113460 | 113492 | 37, 18, 40 |
| 5 | DND1 | DND microRNA-mediated repression inhibitor 1 | 256453 | 120314 | 170445 | 3, 40, 16 |
| 5 | DOK3 | docking protein 3 | 146094 | 196923 | 183258 | 41, 36, 5 |
| 5 | DRD1 | dopamine receptor D1 | 184845 | 120149 | 164466 | 28, 8, 14 |
| 5 | DBN1 | drebrin 1 | 113758 | 131188 | 196923 | 26, 8, 39 |
| 5 | DROSHA | drosha ribonuclease III | 113360 | 113361 | 082213 | 10, 11, 24 |
| 5 | DTWD2 | DTW domain containing 2 | 169570 | 092421 | 172869 | 33, 10, 42 |
| 5 | DUSP1 | dual specificity phosphatase 1 | 120129 | 214357 | 113719 | 1, 29, 18 |
| 5 | DCTN4 | dynactin subunit 4 | 132912 | 086589 | 256235 | 42, 24, 8 |
| 5 | DNAH5 | dynein axonemal heavy chain 5 | 039139 | 169862 | 038382 | 10, 31, 42 |
| 5 | EBF1 | early B-cell factor 1 | 164330 | 113282 | 145860 | 22, 18, 11 |
| 5 | EGR1 | early growth response 1 | 120738 | 132563 | 120705 | 40, 26, 11 |
| 5 | ENC1 | ectodermal-neural cortex 1 | 171617 | 214944 | 049860 | 8, 29, 45 |
| 5 | EFCAB9 | EF-hand calcium binding domain 9 | 214360 | 072786 | 168246 | 38, |
| 5 | EDIL3 | EGF like repeats and discoidin domains 3 | 164176 | 145681 | 127184 | 18, 8, 42 |
| 5 | EGFLAM | EGF like, fibronectin type III and laminin G domains | 164318 | 168621 | 113594 | 26, 32, 33 |
| 5 | ELL2 | elongation factor for RNA polymerase II 2 | 118985 | 173221 | 175426 | 26, 40, 1 |
| 5 | ELOVL7 | ELOVL fatty acid elongase 7 | 164181 | 035499 | 049167 | 29, 26, 2 |
| 5 | EMB | embigin | 170571 | 164588 | 151883 | 40, 22, 42 |
| 5 | ERAP1 | endoplasmic reticulum aminopeptidase 1 | 164307 | 153113 | 164308 | 1, 42, 39 |
| 5 | ERAP2 | endoplasmic reticulum aminopeptidase 2 | 164308 | 164307 | 113441 | 42, 39, 41 |
| 5 | ERGIC1 | endoplasmic reticulum-golgi intermediate compartment 1 | 113719 | 120129 | 037241 | 33, 2, 16 |
| 5 | ESM1 | endothelial cell specific molecule 1 | 164283 | 178996 | 113088 | 45, 43, 4 |
| 5 | ECSCR | endothelial cell surface expressed chemotaxis and apoptosis | 249751 | 279686 | 283288 | 43, 26, 15 |
| | | regulator | | | | |
| 5 | EFNA5 | ephrin A5 | 184349 | 112874 | 145743 | 6, 31, 27 |
| 5 | ERBIN | erbb2 interacting protein | 112851 | 123213 | 153914 | |
| 5 | ERCC8 | ERCC excision repair 8, CSA ubiquitin ligase complex subunit | 049167 | 164181 | 164182 | 7, 4, 38 |
| 5 | EPB41L4A | erythrocyte membrane protein band 4.1 like 4A | 129595 | 134986 | 134982 | 40, 18, 26 |
| 5 | EIF4EBP3 | eukaryotic translation initiation factor 4E binding protein 3 | 243056 | 213523 | 113108 | |
| 5 | EIF4E1B | eukaryotic translation initiation factor 4E family member 1B | 175766 | 074317 | 048140 | 8, 14, 38 |
| 5 | ETF1 | eukaryotic translation termination factor 1 | 120705 | 120738 | 113013 | 13, 5, 40 |
| 5 | EXOC3 | exocyst complex component 3 | 180104 | 063438 | 066230 | 18, 19, 26 |
| 5 | F2RL1 | F2R like trypsin receptor 1 | 164251 | 181104 | 171643 | 29, 31, 12 |
| 5 | FAM105A | family with sequence similarity 105 member A | 145569 | 281325 | 154124 | 9, 18, 31 |
| 5 | FAM114A2 | family with sequence similarity 114 member A2 | 055147 | 155511 | 037749 | 38, |
| 5 | FAM13B | family with sequence similarity 13 member B | 031003 | 078795 | 061492 | 22, 2, 26 |
| 5 | FAM134B | family with sequence similarity 134 member B | 154153 | 173545 | 145555 | 29, 26, 17 |
| 5 | FAM151B | family with sequence similarity 151 member B | 152380 | 039319 | 189127 | 10, 17, 14 |
| 5 | FAM153A | family with sequence similarity 153 member A | 170074 | 027847 | 175325 | |
| 5 | FAM153B | family with sequence similarity 153 member B | 182230 | 051596 | 170085 | 1, 38, 9 |
| 5 | FAM153C | Family with sequence similarity 153 member C | 204677 | 175325 | 145911 | 28, 38, 33 |
| 5 | FAM159B | family with sequence similarity 159 member B | 145642 | 186479 | 153006 | 28, 10, 44 |
| 5 | FAM169A | family with sequence similarity 169 member A | 198780 | 164346 | 176928 | 14, 31, 42 |
| 5 | FAM170A | family with sequence similarity 170 member A | 164334 | 133835 | 184838 | |
| 5 | FAM172A | family with sequence similarity 172 member A | 113391 | 175745 | 248483 | 29, 31, 8 |
| 5 | FAM173B | family with sequence similarity 173 member B | 150756 | 169777 | 150753 | 29, 31, 10 |
| 5 | FAM174A | family with sequence similarity 174 member A | 174132 | 153922 | 113532 | 31, 10, 38 |
| 5 | FAM193B | family with sequence similarity 193 member B | 146067 | 183258 | 184840 | 18, 39, 1 |
| 5 | FAM196B | family with sequence similarity 196 member B | 204767 | 134516 | 168269 | 45, 8, 37 |
| 5 | FAM53C | family with sequence similarity 53 member C | 120709 | 158402 | 120733 | 42, 38, 9 |
| 5 | FAM71B | family with sequence similarity 71 member B | 170613 | 113263 | 055163 | 38, 8, |
| 5 | FAM81B | family with sequence similarity 81 member B | 153347 | 175471 | 198677 | 10, 30, 38 |
| 5 | FAF2 | Fas associated factor family member 2 | 113194 | 175416 | 146083 | 11, 3, 15 |
| 5 | FASTKD3 | FAST kinase domains 3 | 124279 | 124275 | 112902 | 43, 4, 42 |
| 5 | FAT2 | FAT atypical cadherin 2 | 086570 | 123643 | 113140 | 29, 24, 31 |
| 5 | FABP6 | fatty acid binding protein 6 | 170231 | 170234 | 135083 | 18, 10, 17 |
| 5 | FAXDC2 | fatty acid hydroxylase domain containing 2 | 170271 | 155506 | 155508 | 26, 43, 8 |
| 5 | FBXL17 | F-box and leucine rich repeat protein 17 | 145743 | 184349 | 151422 | 11, 33, 4 |
| 5 | FBXL7 | F-box and leucine rich repeat protein 7 | 183580 | 154122 | 183654 | 34, 15, 26 |
| 5 | FBXW11 | F-box and WD repeat domain containing 11 | 072803 | 185662 | 072786 | 43, 9, 26 |
| 5 | FBXO38 | F-box protein 38 | 145868 | 204909 | 164270 | 42, 22, 39 |
| 5 | FBXO4 | F-box protein 4 | 151876 | 205765 | 112964 | 18, 37, 22 |
| 5 | FCHSD1 | FCH and double SH3 domains 1 | 197948 | 164620 | 120318 | 29, 36, 22 |
| 5 | FCHO2 | FCH domain only 2 | 157107 | 083312 | 157111 | 4, 2, 42 |
| 5 | FEM1C | fem-1 homolog C | 145780 | 164221 | 251201 | 42, 7, 22 |
| 5 | FER | FER tyrosine kinase | 151422 | 145743 | 198961 | 36, 19, 4 |
| 5 | FTMT | ferritin mitochondrial | 181867 | 184838 | 151304 | 38, 8, |
| 5 | FBLL1 | Fibrillarin-like 1 | 188573 | 113643 | 120137 | 28, 38, 44 |
| 5 | FBN2 | fibrillin 2 | 138829 | 064651 | 113396 | 32, 12, 15 |
| 5 | FGF1 | fibroblast growth factor 1 | 113578 | 187678 | 145819 | |
| 5 | FGF10 | fibroblast growth factor 10 | 070193 | 112992 | 112996 | 42, 25, 16 |
| 5 | FGF18 | fibroblast growth factor 18 | 156427 | 181163 | 185662 | 27, 20, 15 |
| 5 | FGFR4 | fibroblast growth factor receptor 4 | 160867 | 113761 | 165671 | 25, 21, 16 |
| 5 | FNDC9 | fibronectin type III domain containing 9 | 172568 | 055163 | 135074 | 8, 17, 14 |
| 5 | FLT4 | fms related tyrosine kinase 4 | 037280 | 161055 | 174339 | 22, 32, 6 |
| 5 | FNIP1 | folliculin interacting protein 1 | 217128 | 273217 | 239642 | 22, 1, 19 |
| 5 | FST | follistatin | 134363 | 164172 | 164258 | 27, 11, 18 |
| 5 | FSTL4 | follistatin like 4 | 053108 | 170606 | 113583 | 28, 14, 24 |
| 5 | FOXD1 | forkhead box D1 | 251493 | 164325 | 145741 | |
| 5 | FOXI1 | forkhead box 11 | 168269 | 204767 | 234511 | 23, 17, 20 |
| 5 | FYB | FYN binding protein | 082074 | 164327 | 113600 | 39, 30, 7 |
| 5 | GFM2 | G elongation factor mitochondrial 2 | 164347 | 049860 | 164346 | 39, 42, 4 |
| 5 | GPRIN1 | G protein regulated inducer of neurite outgrowth 1 | 169258 | 074276 | 074317 | 31, 8, 25 |
| 5 | GPR150 | G protein-coupled receptor 150 | 178015 | 164291 | 175449 | 29, 38, |
| 5 | GPR151 | G protein-coupled receptor 151 | 173250 | 113649 | 156475 | |
| 5 | GRK6 | G protein-coupled receptor kinase 6 | 198055 | 131187 | 131188 | 5, 41, 3 |
| 5 | G3BP1 | G3BP stress granule assembly factor 1 | 145907 | 177556 | 145888 | 13, 45, 24 |
| 5 | GABRA1 | gamma-aminobutyric acid type A receptor alpha1 subunit | 022355 | 145863 | 113327 | 8, 2, 17 |
| 5 | GABRA6 | gamma-aminobutyric acid type A receptor alpha6 subunit | 145863 | 145864 | 022355 | 38, |
| 5 | GABRB2 | gamma-aminobutyric acid type A receptor beta2 subunit | 145864 | 118322 | 145863 | 8, 31, 5 |
| 5 | GABRG2 | gamma-aminobutyric acid type A receptor gamma2 subunit | 113327 | 022355 | 113328 | 8, 26, 18 |
| 5 | GABRP | gamma-aminobutyric acid type A receptor pi subunit | 094755 | 145936 | 204764 | 24, 42, 23 |
| 5 | GPBP1 | GC-rich promoter binding protein 1 | 062194 | 155545 | 169067 | 37, 38, 25 |
| 5 | GFRA3 | GDNF family receptor alpha 3 | 146013 | 094880 | 158402 | 26, 11, 15 |
| 5 | GEMIN5 | gem nuclear organelle associated protein 5 | 082516 | 155508 | 082515 | 21, 3, 1 |
| 5 | GTF2H2 | general transcription factor IIH subunit 2 | 145736 | 249437 | 145734 | 34, 31, 13 |
| 5 | GDNF | glial cell derived neurotrophic factor | 168621 | 082068 | 164318 | 35, 27, 25 |
| 5 | GNPDA1 | glucosamine-6-phosphate deaminase 1 | 113552 | 013561 | 131507 | 27, 4, 11 |
| 5 | GCNT4 | glucosaminyl (N-acetyl) transferase 4, core 2 | 176928 | 198780 | 145700 | 32, 20, |
| 5 | GRIA1 | glutamate ionotropic receptor AMPA type subunit 1 | 155511 | 132911 | 055147 | |
| 5 | GRM6 | glutamate metabotropic receptor 6 | 113262 | 178187 | 234284 | |
| 5 | GFPT2 | glutamine-fructose-6-phosphate transaminase 2 | 131459 | 050748 | 113300 | 11, 35, 10 |
| 5 | GLRX | glutaredoxin | 173221 | 164292 | 118985 | 1, 2, 32 |
| 5 | GRXCR2 | glutaredoxin and cysteine rich domain containing 2 | 204928 | 186314 | 156463 | |
| 5 | GPX3 | glutathione peroxidase 3 | 211445 | 279109 | 145901 | 40, 10, 14 |
| 5 | GPX8 | glutathione peroxidase 8 (putative) | 164294 | 164287 | 234602 | 42, 6, 10 |
| 5 | GLRA1 | glycine receptor alpha 1 | 145888 | 145907 | 132911 | 38, 8, |
| 5 | GM2A | GM2 ganglioside activator | 196743 | 198624 | 186334 | 18, 5, 36 |
| 5 | GOLPH3 | golgi phosphoprotein 3 | 113384 | 133401 | 150712 | 4, 42, 26 |
| 5 | GRAMD3 | GRAM domain containing 3 | 155324 | 168916 | 164904 | |
| 5 | GZMA | granzyme A | 145649 | 113088 | 164287 | 10, 30, 17 |
| 5 | GZMK | granzyme K | 113088 | 164283 | 145649 | 30, 40, 39 |
| 5 | GAPT | GRB2 binding adaptor protein, transmembrane | 175857 | 145632 | 152932 | 7, 1, 30 |
| 5 | GDF9 | growth differentiation factor 9 | 164404 | 164403 | 164405 | 2, 38, 23 |
| 5 | GHR | growth hormone receptor | 112964 | 151876 | 198865 | 26, 29, 1 |
| 5 | GRPEL2 | GrpE like 2, mitochondrial | 164284 | 157510 | 145882 | 11, 42, 24 |
| 5 | GTF2H2C | GTF2H2 family member C | 183474 | 197822 | 205572 | |
| 5 | GIN1 | gypsy retrotransposon integrase 1 | 145723 | 145730 | 145725 | 29, 4, 31 |
| 5 | H2AFY | H2A histone family member Y | 113648 | 069011 | 251380 | 5, 31, 7 |
| 5 | HAND1 | heart and neural crest derivatives expressed 1 | 113196 | 164576 | 155506 | 26, 12, 14 |
| 5 | HSPA4 | heat shock protein family A (Hsp70) member 4 | 170606 | 155329 | 053108 | 37, 3, 24 |
| 5 | HSPA9 | heat shock protein family A (Hsp70) member 9 | 113013 | 120705 | 044115 | 3, 40, 24 |
| 5 | HSPB3 | heat shock protein family B (small) member 3 | 169271 | 185305 | 178996 | 15, 20, 25 |
| 5 | HBEGF | heparin binding EGF like growth factor | 113070 | 281227 | 113073 | 26, 13, 15 |
| 5 | HAVCR1 | hepatitis A virus cellular receptor 1 | 113249 | 145850 | 135077 | 16, 17, 14 |
| 5 | HAVCR2 | hepatitis A virus cellular receptor 2 | 135077 | 113249 | 155868 | 5, 36, 10 |
| 5 | HNRNPAB | heterogeneous nuclear ribonucleoprotein A/B | 197451 | 145912 | 175309 | 21, 4, 12 |
| 5 | HNRNPA0 | heterogeneous nuclear ribonucleoprotein A0 | 177733 | 146021 | 120729 | 26, 12, 37 |
| 5 | HNRNPH1 | heterogeneous nuclear ribonucleoprotein H1 | 169045 | 176783 | 204659 | 39, 13, 45 |
| 5 | HK3 | hexokinase 3 | 160883 | 113763 | 087206 | 5, 1, 20 |
| 5 | HEXB | hexosaminidase subunit beta | 049860 | 171617 | 164347 | 45, 32, 17 |
| 5 | HIGD2A | HIG1 hypoxia inducible domain family member 2A | 146066 | 048162 | 175416 | 33, 15, 21 |
| 5 | HRH2 | histamine receptor H2 | 113749 | 164466 | 145920 | 27, 17, 35 |
| 5 | HINT1 | histidine triad nucleotide binding protein 1 | 169567 | 198108 | 186687 | 24, 34, 4 |
| 5 | HARS | histidyl-tRNA synthetase | 170445 | 256453 | 112855 | 2, 13, 8 |
| 5 | HARS2 | histidyl-tRNA synthetase 2, mitochondrial | 112855 | 170445 | 146007 | 21, 4, 35 |
| 5 | HMHB1 | histocompatibility minor HB-1 | 158497 | 113580 | 145817 | 10, |
| 5 | HDAC3 | histone deacetylase 3 | 171720 | 131504 | 164620 | 41, 9, 27 |
| 5 | HMGXB3 | HMG-box containing 3 | 113716 | 164296 | 182578 | 29, 34, 4 |
| 5 | HMP19 | HMP19 protein | 170091 | 185056 | 120149 | 8, 26, 31 |
| 5 | HOMER1 | homer scaffolding protein 1 | 152413 | 152409 | 164329 | 32, 26, 34 |
| 5 | HAPLN1 | hyaluronan and proteoglycan link protein 1 | 145681 | 038427 | 164176 | 11, 6, 12 |
| 5 | HMMR | hyaluronan mediated motility receptor | 072571 | 170584 | 038274 | 10, 39, 3 |
| 5 | HSD17B4 | hydroxysteroid 17-beta dehydrogenase 4 | 133835 | 145779 | 164334 | 1, 29, 2 |
| 5 | HCN1 | hyperpolarization activated cyclic nucleotide gated potassium | 164588 | 112996 | 170571 | 14, 25, 8 |
| | | channel 1 | | | | |
| 5 | IGIP | IgA inducing protein | 182700 | 185129 | 120306 | 1, 11, 29 |
| 5 | IK | IK cytokine, down-regulator of HLA II | 113141 | 113119 | 120314 | 30, 13, 4 |
| 5 | ITK | IL2 inducible T-cell kinase | 113263 | 155868 | 170613 | 39, 5, 42 |
| 5 | IRGM | immunity related GTPase M | 237693 | 256235 | 145908 | |
| 5 | IPO11 | importin 11 | 086200 | 086189 | 186105 | 42, 7, 38 |
| 5 | ITGA1 | integrin subunit alpha 1 | 213949 | 016082 | 152684 | 21, 10, 17 |
| 5 | ITGA2 | integrin subunit alpha 2 | 164171 | 152684 | 164172 | 42, 4, 3 |
| 5 | ICE1 | interactor of little elongation complex ELL subunit 1 | 164151 | 145536 | 133398 | 19, 18, 12 |
| 5 | IRF1 | interferon regulatory factor 1 | 125347 | 197536 | 113525 | 41, 44, 36 |
| 5 | IL12B | interleukin 12B | 113302 | 164332 | 170214 | |
| 5 | IL13 | interleukin 13 | 169194 | 113522 | 113520 | 7, 38, 8 |
| 5 | IL17B | interleukin 17B | 127743 | 145882 | 113712 | |
| 5 | IL3 | interleukin 3 | 164399 | 164398 | 164400 | |
| 5 | IL31RA | interleukin 31 receptor A | 164509 | 152670 | 279768 | 29, 45, 6 |
| 5 | IL4 | interleukin 4 | 113520 | 169194 | 131437 | 5, 36, |
| 5 | IL5 | interleukin 5 | 113525 | 125347 | 113522 | 39, |
| 5 | IL6ST | interleukin 6 signal transducer | 134352 | 279768 | 164512 | 29, 42, 26 |
| 5 | IL7R | interleukin 7 receptor | 168685 | 152582 | 152611 | 39, 5, 18 |
| 5 | IL9 | interleukin 9 | 145839 | 145832 | 145826 | 17, |
| 5 | IQGAP2 | IQ motif containing GTPase activating protein 2 | 145703 | 254893 | 164220 | 38, 11, 34 |
| 5 | IRX1 | iroquois homeobox 1 | 170549 | 186493 | 145536 | 26, 11, 15 |
| 5 | IRX2 | iroquois homeobox 2 | 170561 | 113430 | 186493 | 20, 28, 17 |
| 5 | IRX4 | iroquois homeobox 4 | 113430 | 145494 | 170561 | 18, 32, 27 |
| 5 | ISL1 | ISL LIM homeobox 1 | 016082 | 151883 | 213949 | 43, 14, 28 |
| 5 | ISOC1 | isochorismatase domain containing 1 | 066583 | 113396 | 145808 | 9, 4, 34 |
| 5 | JADE2 | jade family PHD finger 2 | 043143 | 237190 | 152700 | 18, 22, 39 |
| 5 | JAKMIP2 | janus kinase and microtubule interacting protein 2 | 176049 | 113657 | 164266 | 11, 26, 6 |
| 5 | JMY | junction mediating and regulatory protein, p53 cofactor | 152409 | 145692 | 152413 | 11, 29, 42 |
| 5 | KLHL3 | kelch like family member 3 | 146021 | 152377 | 177733 | 11, 40, 1 |
| 5 | KIAA0141 | KIAA0141 | 081791 | 156453 | 113555 | 22, 7, 29 |
| 5 | KIAA0825 | KIAA0825 | 185261 | 248483 | 133302 | 40, 45, 23 |
| 5 | KIAA1024L | KIAA1024like | 186367 | 145808 | 198108 | |
| 5 | KIAA1191 | KIAA1191 | 122203 | 170085 | 175414 | 29, 45, 12 |
| 5 | KIF20A | kinesin family member 20A | 112984 | 112983 | 094880 | 24, 45, 12 |
| 5 | KIF2A | kinesin family member 2A | 068796 | 130449 | 086189 | 22, 7, 39 |
| 5 | KIF3A | kinesin family member 3A | 131437 | 113520 | 205089 | 31, 26, 8 |
| 5 | KIF4B | kinesin family member 4B | 226650 | 082515 | 170624 | 38, 6, 22 |
| 5 | LARP1 | La ribonucleoprotein domain family member 1 | 155506 | 113196 | 170271 | 22, 23, 21 |
| 5 | LVRN | laeverin | 172901 | 280329 | 268714 | 32, 26, 44 |
| 5 | LMNB1 | lamin B1 | 113368 | 196900 | 173926 | 39, 4, 7 |
| 5 | LMAN2 | lectin, mannose binding 2 | 169223 | 169230 | 169220 | 27, 34, 28 |
| 5 | LRRC14B | leucine rich repeat containing 14B | 185028 | 153404 | 164366 | 14, |
| 5 | LRRC70 | leucine rich repeat containing 70 | 186105 | 086200 | 178394 | 1, 7, 5 |
| 5 | LRRTM2 | leucine rich repeat transmembrane neuronal 2 | 146006 | 044115 | 120725 | 8, 26, 14 |
| 5 | LNPEP | leucyl and cystinyl aminopeptidase | 113441 | 164308 | 145721 | 28, 33, 44 |
| 5 | LARS | leucyl-tRNA synthetase | 133706 | 173261 | 275740 | 42, 31, 24 |
| 5 | LIFR | leukemia inhibitory factor receptor alpha | 113594 | 164318 | 145623 | |
| 5 | LECT2 | leukocyte cell derived chemotaxin 2 | 145826 | 145839 | 120708 | 19, 38, |
| 5 | LTC4S | leukotriene C4 synthase | 213316 | 161021 | 161013 | 17, 6, 38 |
| 5 | LIX1 | limb and CNS expressed 1 | 145721 | 113441 | 058729 | 26, 8, 12 |
| 5 | LHFPL2 | lipoma HMGIC fusion partner-like 2 | 145685 | 085365 | 113273 | |
| 5 | LEAP2 | liver enriched antimicrobial peptide 2 | 164406 | 164405 | 072364 | 19, 35, 23 |
| 5 | LMBRD2 | LMBR1 domain containing 2 | 164187 | 168671 | 145604 | 40, 22, 25 |
| 5 | LSM11 | LSM11, U7 small nuclear RNA associated | 155858 | 113272 | 113282 | 27, 4, 9 |
| 5 | LCP2 | lymphocyte cytosolic protein 2 | 043462 | 234511 | 182132 | 5, 39, 36 |
| 5 | LYRM7 | LYR motif containing 7 | 186687 | 169567 | 158985 | 26, 4, 29 |
| 5 | KDM3B | lysine demethylase 3B | 120733 | 120709 | 132563 | 26, 31, 18 |
| 5 | LYSMD3 | LysM domain containing 3 | 176018 | 113356 | 164199 | 22, 18, 1 |
| 5 | LPCAT1 | lysophosphatidylcholine acyltransferase 1 | 153395 | 142319 | 171421 | 1, 33, 3 |
| 5 | LOX | lysyl oxidase | 113083 | 151304 | 164185 | 45, 6, 10 |
| 5 | MROH2B | maestro heat like repeat family member 2 B | 171495 | 112936 | 039537 | 38, 10, 19 |
| 5 | MAN2A1 | mannosidase alpha class 2A member 1 | 112893 | 198961 | 186952 | 1, 45, 11 |
| 5 | MGAT1 | mannosyl (alpha-1,3-)-glycoprotein | 131446 | 174339 | 196670 | 24, 36, 39 |
| | | beta-1,2-N-acetylglucosaminyltransferase | | | | |
| 5 | MGAT4B | mannosyl (alpha-1,3-)-glycoprotein | 161013 | 213316 | 161011 | 34, 28, 35 |
| | | beta-1,4-N-acetylglucosaminyltransferase, isozyme B | | | | |
| 5 | MZB1 | marginal zone B and B1 cell specific protein | 170476 | 170482 | 228672 | 39, 41, 22 |
| 5 | MARVELD2 | MARVEL domain containing 2 | 152939 | 152942 | 197822 | 29, 23, 18 |
| 5 | MAML1 | mastermind like transcriptional coactivator 1 | 161021 | 127022 | 213316 | 22, 1, 7 |
| 5 | MATR3 | matrin 3 | 015479 | 280987 | 120727 | 42, 25, 8 |
| 5 | MATR3 | matrin 3 | 280987 | 120725 | 015479 | 42, 25, 8 |
| 5 | MXD3 | MAX dimerization protein 3 | 213347 | 169228 | 169230 | 37, 41, 36 |
| 5 | MED10 | mediator complex subunit 10 | 133398 | 164151 | 215218 | 6, 34, 10 |
| 5 | MED7 | mediator complex subunit 7 | 155868 | 135077 | 113263 | |
| 5 | MEIKIN | meiotic kinetochore factor | 239642 | 217128 | 281938 | |
| 5 | MARCH11 | membrane associated ring-CH-type finger 11 | 183654 | 183580 | 173545 | 38, 10, 32 |
| 5 | MARCH3 | membrane associated ring-CH-type finger 3 | 173926 | 113368 | 164241 | 10, 38, 14 |
| 5 | MARCH6 | membrane associated ring-CH-type finger 6 | 145495 | 164237 | 145491 | 11, 39, 42 |
| 5 | MBLAC2 | metallo-beta-lactamase domain containing 2 | 176055 | 153140 | 113356 | 7, 42, 39 |
| 5 | MTX3 | metaxin 3 | 177034 | 164309 | 113296 | 30, 1, 26 |
| 5 | MAT2B | methionine adenosyltransferase 2B | 038274 | 072571 | 145934 | 43, 4, 7 |
| 5 | MCCC2 | methylcrotonoyl-CoA carboxylase 2 | 131844 | 145734 | 164326 | 33, 26, 4 |
| 5 | MFAP3 | microfibrillar associated protein 3 | 037749 | 055147 | 164574 | 42, 2, 32 |
| 5 | MAPI B | microtubule associated protein 1B | 131711 | 164326 | 113048 | 26, 8, 33 |
| 5 | MAST4 | microtubule associated serine/threonine kinase family member 4 | 069020 | 153914 | 134061 | 30, 2, 18 |
| 5 | MIER3 | MIER family member 3 | 155545 | 155542 | 062194 | 23, 4, 22 |
| 5 | MRPL22 | mitochondrial ribosomal protein L22 | 082515 | 082516 | 226650 | 10, 43, 31 |
| 5 | MRPL36 | mitochondrial ribosomal protein L36 | 171421 | 153395 | 145494 | 2, 26, 7 |
| 5 | MRPS27 | mitochondrial ribosomal protein S27 | 113048 | 131711 | 049883 | 21, 9, 3 |
| 5 | MRPS30 | mitochondrial ribosomal protein S30 | 112996 | 070193 | 164588 | 9, 43, 11 |
| 5 | MRPS36 | mitochondrial ribosomal protein S36 | 134056 | 153044 | 134058 | 11, 1, 31 |
| 5 | MAPK9 | mitogen-activated protein kinase 9 | 050748 | 146090 | 131459 | 29, 22, 43 |
| 5 | MAP3K1 | mitogen-activated protein kinase kinase kinase 1 | 095015 | 225940 | 155542 | 22, 40, 1 |
| 5 | MOCS2 | molybdenum cofactor synthesis 2 | 164172 | 164171 | 134363 | 29, 42, 34 |
| 5 | MRNIP | MRN complex interacting protein | 161010 | 161011 | 197226 | |
| 5 | MSX2 | msh homeobox 2 | 120149 | 170091 | 184845 | 29, 12, 32 |
| 5 | MCIDAS | multiciliate differentiation and DNA synthesis associated | 234602 | 164294 | 152669 | 9, 5, 23 |
| | | cell cycle protein | | | | |
| 5 | MCTP1 | multiple C2 and transmembrane domain containing 1 | 175471 | 133302 | 153347 | 43, 39, 26 |
| 5 | MEGF10 | multiple EGF like domains 10 | 145794 | 164241 | 164244 | 26, 8, 25 |
| 5 | MCC | mutated in colorectal cancers | 171444 | 172795 | 212122 | 11, 14, 35 |
| 5 | MSH3 | mutS homolog 3 | 113318 | 228716 | 113319 | 43, 17, 26 |
| 5 | MEF2C | myocyte enhancer factor 2C | 081189 | 164180 | 153140 | 22, |
| 5 | MYO10 | myosin X | 145555 | 154153 | 176788 | 35, 18, 31 |
| 5 | MYOT | myotilin | 120729 | 177733 | 078795 | 25, 15, 18 |
| 5 | MTMR12 | myotubularin related protein 12 | 150712 | 113384 | 056097 | 34, 40, 22 |
| 5 | MYOZ3 | myozenin 3 | 164591 | 171992 | 086589 | 25, 24, 33 |
| 5 | NADK2 | NAD kinase 2, mitochondrial | 152620 | 145604 | 164188 | 22, 19, 31 |
| 5 | NDUFAF2 | NADH: ubiquinone oxidoreductase complex assembly factor 2 | 164182 | 049167 | 188725 | 10, 31, 22 |
| 5 | NDUFA2 | NADH: ubiquinone oxidoreductase subunit A2 | 131495 | 170458 | 113119 | 29, 31, 15 |
| 5 | NDUFS4 | NADH: ubiquinone oxidoreductase subunit S4 | 164258 | 134363 | 185305 | 15, 29, 2 |
| 5 | NDUFS6 | NADH: ubiquinone oxidoreductase subunit S6 | 145494 | 171421 | 113430 | 25, 33, 2 |
| 5 | NKD2 | naked cuticle homolog 2 | 145506 | 071539 | 113504 | 23, 6, 14 |
| 5 | NPR3 | natriuretic peptide receptor 3 | 113389 | 113387 | 113407 | 7, 17, 27 |
| 5 | NDST1 | N-deacetylase and N-sulfotransferase 1 | 070614 | 164587 | 171992 | 40, 1, 27 |
| 5 | N4BP3 | NEDD4 binding protein 3 | 145911 | 204677 | 145916 | 18, 32, 22 |
| 5 | NDFIP1 | Nedd4 family interacting protein 1 | 131507 | 113552 | 187678 | 29, 8, 11 |
| 5 | NEURL1B | neuralized E3 ubiquitin protein ligase 1B | 214357 | 174705 | 120129 | 11, 1, 44 |
| 5 | NRG2 | neuregulin 2 | 158458 | 146005 | 185129 | 29, 31, 27 |
| 5 | NEUROG1 | neurogenin 1 | 181965 | 255833 | 145824 | 14, 8, |
| 5 | NLN | neurolysin | 123213 | 197860 | 112851 | 43, 10, 13 |
| 5 | NMUR2 | neuromedin U receptor 2 | 132911 | 145888 | 155511 | 3, 15, 32 |
| 5 | NREP | neuronal regeneration related protein | 134986 | 164211 | 129595 | 11, 15, 12 |
| 5 | NHP2 | NHP2 ribonucleoprotein | 145912 | 145916 | 197451 | 29, 3, 9 |
| 5 | NNT | nicotinamide nucleotide transhydrogenase | 112992 | 172239 | 070193 | 22, 7, 2 |
| 5 | NIM1K | NIM1 serine/threonine protein kinase | 177453 | 172262 | 112972 | 34, 8, 32 |
| 5 | NIPAL4 | NIPA like domain containing 4 | 172548 | 135074 | 039600 | 4, 24, 15 |
| 5 | NIPBL | NIPBL, cohesin loading factor | 164190 | 079215 | 197603 | 29, 24, 26 |
| 5 | NKX2-5 | NK2 homeobox 5 | 183072 | 113734 | 113739 | 15, 36, 12 |
| 5 | NAIP | NLR family apoptosis inhibitory protein | 249437 | 172062 | 145736 | |
| 5 | NME5 | NME/NM23 family member 5 | 112981 | 061492 | 112983 | 29, 27, 38 |
| 5 | NOP16 | NOP16 nucleolar protein | 048162 | 175414 | 146066 | 3, 29, 22 |
| 5 | NSUN2 | NOP2/Sun RNA methyltransferase family member 2 | 037474 | 215218 | 145545 | 29, 6, 12 |
| 5 | NSA2 | NSA2, ribosome biogenesis homolog | 164346 | 164347 | 198780 | 34, 25, 1 |
| 5 | NSD1 | nuclear receptor binding SET domain protein 1 | 165671 | 160867 | 169228 | 26, 1, 40 |
| 5 | NR2F1 | nuclear receptor subfamily 2 group F member 1 | 175745 | 113369 | 113391 | |
| 5 | NR3C1 | nuclear receptor subfamily 3 group C member 1 | 113580 | 145819 | 158497 | 31, 45, 22 |
| 5 | NPM1 | nucleophosmin | 181163 | 164438 | 156427 | 7, 4, 34 |
| 5 | NUP155 | nucleoporin 155 | 113569 | 197603 | 082068 | 38, 31, 24 |
| 5 | NUDCD2 | NudC domain containing 2 | 170584 | 113328 | 072571 | 29, 4, 18 |
| 5 | NUDT12 | nudix hydrolase 12 | 112874 | 181751 | 184349 | 42, 31, 2 |
| 5 | OCLN | occludin | 197822 | 152939 | 183474 | 43, 13, 29 |
| 5 | OR2V1 | olfactory receptor family 2 subfamily V member 1 | 185372 | 165810 | 182613 | |
| 5 | OR2V2 | olfactory receptor family 2 subfamily V member 2 | 182613 | 185372 | 146054 | |
| 5 | OR2Y1 | olfactory receptor family 2 subfamily Y member 1 | 174339 | 037280 | 131446 | |
| 5 | OR4F3 | olfactory receptor family 4 subfamily F member 3 | 230178 | 183718 | 176049 | |
| 5 | OSMR | oncostatin M receptor | 145623 | 113594 | 164327 | 18, 42, 13 |
| 5 | OTP | orthopedia homeobox | 171540 | 164253 | 171530 | 20, 14, 38 |
| 5 | OTULIN | OTU deubiquitinase with linear linkage specificity | 154124 | 145569 | 154122 | 6, 24, 2 |
| 5 | PITX1 | paired like homeodomain 1 | 069011 | 152705 | 113648 | 31, 9, 3 |
| 5 | PANK3 | pantothenate kinase 3 | 120137 | 188573 | 184347 | |
| 5 | PDLIM4 | PDZ and LIM domain 4 | 131435 | 072682 | 197208 | 27, 26, 35 |
| 5 | PDLIM7 | PDZ and LIM domain 7 | 196923 | 113758 | 146094 | 41, 33, 35 |
| 5 | PDZD2 | PDZ domain containing 2 | 133401 | 082213 | 113384 | 24, 26, 32 |
| 5 | PELO | pelota homolog (Drosophila) | 152684 | 213949 | 164171 | 21, 10, 17 |
| 5 | PTCD2 | pentatricopeptide repeat domain 2 | 049883 | 113048 | 178175 | 26, 45, 6 |
| 5 | PAM | peptidylglycine alpha-amidating monooxygenase | 145730 | 205359 | 145723 | |
| 5 | PPIC | peptidylprolyl isomerase C | 168938 | 064652 | 061455 | 18, 11, 6 |
| 5 | PPWD1 | peptidylprolyl isomerase domain and WD repeat containing 1 | 113593 | 123219 | 113595 | 13, 4, 42 |
| 5 | PLCXD3 | phosphatidylinositol specific phospholipase C X domain | 182836 | 039537 | 083720 | 28, 26, 2 |
| | | containing 3 | | | | |
| 5 | PDE4D | phosphodiesterase 4D | 113448 | 152932 | 280447 | |
| 5 | PDE6A | phosphodiesterase 6A | 132915 | 155846 | 155850 | 14, 25, 6 |
| 5 | PDE8B | phosphodiesterase 8B | 113231 | 132846 | 164253 | 40, 24, 28 |
| 5 | PIK3R1 | phosphoinositide-3-kinase regulatory subunit 1 | 145675 | 134061 | 145740 | 8, 44, 36 |
| 5 | PLPP1 | phospholipid phosphatase 1 | 067113 | 039123 | 177058 | |
| 5 | PHAX | phosphorylated adaptor for RNA export | 164902 | 164904 | 196900 | 18, 4, 3 |
| 5 | PTTG1 | pituitary tumor-transforming 1 | 164611 | 164609 | 118322 | 34, 7, 4 |
| 5 | PLAC8L1 | PLAC8 like 1 | 173261 | 156463 | 133706 | 10, 38, |
| 5 | PDGFRB | platelet derived growth factor receptor beta | 113721 | 182578 | 113722 | 26, 6, 18 |
| 5 | PSD2 | pleckstrin and Sec7 domain containing 2 | 146005 | 171604 | 158458 | 26, 8, 24 |
| 5 | PLEKHG4B | pleckstrin homology and RhoGEF domain containing G4B | 153404 | 176049 | 185028 | 31, 30, 12 |
| 5 | POC5 | POC5 centriolar protein | 152359 | 189045 | 122012 | 33, 13, 29 |
| 5 | PLK2 | polo like kinase 2 | 145632 | 169067 | 175857 | 36, 3, 45 |
| 5 | PAIP1 | poly(A) binding protein interacting protein 1 | 172239 | 172244 | 112992 | 18, 2, 32 |
| 5 | PAIP2 | poly(A) binding protein interacting protein 2 | 120727 | 015479 | 170482 | 29, 31, 1 |
| 5 | PAPD4 | poly(A) RNA polymerase D4, non-canonical | 164329 | 152413 | 164309 | 42, 23, 22 |
| 5 | PAPD7 | poly(A) RNA polymerase D7, non-canonical | 112941 | 145545 | 078295 | 9, 22, 6 |
| 5 | PARP8 | poly(ADP-ribose) polymerase family member 8 | 151883 | 170571 | 016082 | 39, 42, 5 |
| 5 | PKD2L2 | polycystin 2 like 2, transient receptor potential cation | 078795 | 120729 | 031003 | 38, 27, 5 |
| | | channel | | | | |
| 5 | GALNT10 | polypeptide N-acetylgalactosaminyltransferase 10 | 164574 | 037749 | 164576 | 40, 29, 15 |
| 5 | KCNMB1 | potassium calcium-activated channel subfamily M regulatory beta subunit 1 | 145936 | 182132 | 094755 | 1, 44, 45 |
| 5 | KCNN2 | potassium calcium-activated channel subfamily N member 2 | 080709 | 047188 | 152503 | 11, 2, 35 |
| 5 | KCTD16 | potassium channel tetramerization domain containing 16 | 183775 | 145817 | 186314 | 14, 12, 8 |
| 5 | KCNIP1 | potassium voltage-gated channel interacting protein 1 | 182132 | 043462 | 145936 | 28, 8, 30 |
| 5 | POU4F3 | POU class 4 homeobox 3 | 091010 | 091009 | 113649 | |
| 5 | POU5F2 | POU domain class 5, transcription factor 2 | 248483 | 113391 | 185261 | 38, 36, |
| 5 | PPARGC1B | PPARG coactivator 1 beta | 155846 | 183111 | 132915 | 1, 24, 18 |
| 5 | PRDM6 | PR/SET domain 6 | 061455 | 168938 | 168944 | 27, 6, 10 |
| 5 | PRDM9 | PR/SET domain 9 | 164256 | 154162 | 040731 | 38, 25, |
| 5 | PJA2 | praja ring finger ubiquitin ligase 2 | 198961 | 151422 | 112893 | 26, 11, 2 |
| 5 | PFDN1 | prefoldin subunit 1 | 113068 | 120306 | 281227 | 29, 30, 43 |
| 5 | PRELID1 | PRELI domain containing 1 | 169230 | 213347 | 169223 | 9, 21, 28 |
| 5 | PRELID2 | PRELI domain containing 2 | 186314 | 183775 | 204928 | 6, |
| 5 | PCYOX1L | prenylcysteine oxidase 1 like | 145882 | 164284 | 127743 | 7, 29, 5 |
| 5 | PFN3 | profilin 3 | 196570 | 131183 | 131187 | 38, |
| 5 | PDCD6 | programmed cell death 6 | 249915 | 073578 | 063438 | 18, 9, 26 |
| 5 | PRLR | prolactin receptor | 113494 | 113492 | 152582 | 29, 23, 32 |
| 5 | PRR16 | proline rich 16 | 184838 | 164334 | 181867 | 15, 6, 36 |
| 5 | PRR7 | proline rich 7, synaptic | 131188 | 198055 | 113758 | 41, 34, 37 |
| 5 | PROB1 | proline rich basic protein 1 | 228672 | 170476 | 170469 | 39, 22, 36 |
| 5 | PRRC1 | proline rich coiled-coil 1 | 164244 | 145794 | 205279 | 31, 11, 4 |
| 5 | P4HA2 | prolyl 4-hydroxylase subunit alpha 2 | 072682 | 164400 | 131435 | 43, 45, 13 |
| 5 | PROP1 | PROP paired-like homeobox 1 | 175325 | 170074 | 204677 | |
| 5 | PCSK1 | proprotein convertase subtilisin/kexin type 1 | 175426 | 118985 | 153113 | 28, 26, 31 |
| 5 | PTGER4 | prostaglandin E receptor 4 | 171522 | 113638 | 132356 | 22, 1, 42 |
| 5 | PGGT1B | protein geranylgeranyltransferase type I subunit beta | 164219 | 152503 | 164221 | 24, 22, 2 |
| 5 | PRKAA1 | protein kinase AMP-activated catalytic subunit alpha 1 | 132356 | 171522 | 279126 | 42, 29, 11 |
| 5 | PPP1R2P3 | Protein phosphatase 1 regulatory inhibitor subunit 2 | 231989 | 279646 | 145850 | 38, 20, |
| | | pseudogene 3 | | | | |
| 5 | PPP2CA | protein phosphatase 2 catalytic subunit alpha | 113575 | 272772 | 006837 | 4, 2, 29 |
| 5 | PPP2R2B | protein phosphatase 2 regulatory subunit Bbeta | 156475 | 173250 | 169302 | 38, |
| 5 | PCDH1 | protocadherin 1 | 156453 | 120318 | 081791 | 43, 32, 16 |
| 5 | PCDH12 | protocadherin 12 | 113555 | 081791 | 013561 | |
| 5 | PCDHA1 | Protocadherin alpha 1 | 204970 | 146007 | 204969 | |
| 5 | PCDHA10 | Protocadherin alpha 10 | 250120 | 204961 | 249158 | |
| 5 | PCDHA11 | Protocadherin alpha 11 | 249158 | 250120 | 251664 | |
| 5 | PCDHA12 | Protocadherin alpha 12 | 251664 | 249158 | 239389 | |
| 5 | PCDHA13 | Protocadherin alpha 13 | 239389 | 251664 | 248383 | |
| 5 | PCDHA2 | Protocadherin alpha 2 | 204969 | 204970 | 255408 | |
| 5 | PCDHA3 | Protocadherin alpha 3 | 255408 | 204969 | 204967 | |
| 5 | PCDHA4 | Protocadherin alpha 4 | 204967 | 255408 | 204965 | |
| 5 | PCDHA5 | Protocadherin alpha 5 | 204965 | 204967 | 081842 | |
| 5 | PCDHA6 | Protocadherin alpha 6 | 081842 | 204965 | 204963 | |
| 5 | PCDHA7 | Protocadherin alpha 7 | 204963 | 081842 | 204962 | |
| 5 | PCDHA8 | Protocadherin alpha 8 | 204962 | 204963 | 204961 | |
| 5 | PCDHA9 | Protocadherin alpha 9 | 204961 | 204962 | 250120 | 8, 29, 26 |
| 5 | PCDHAC1 | Protocadherin alpha subfamily C, 1 | 248383 | 239389 | 243232 | |
| 5 | PCDHAC2 | Protocadherin alpha subfamily C, 2 | 243232 | 248383 | 171815 | |
| 5 | PCDHB1 | Protocadherin beta 1 | 171815 | 243232 | 112852 | 38, |
| 5 | PCDHB10 | Protocadherin beta 10 | 120324 | 177839 | 197479 | 8, 26, 2 |
| 5 | PCDHB11 | Protocadherin beta 11 | 197479 | 120324 | 120328 | 11, 42, 39 |
| 5 | PCDHB12 | Protocadherin beta 12 | 120328 | 197479 | 187372 | 16, |
| 5 | PCDHB13 | Protocadherin beta 13 | 187372 | 120328 | 120327 | 18, 42, 33 |
| 5 | PCDHB14 | Protocadherin beta 14 | 120327 | 187372 | 279983 | 1, 39, 26 |
| 5 | PCDHB15 | Protocadherin beta 15 | 113248 | 279983 | 178913 | 26, 13, 37 |
| 5 | PCDHB16 | Protocadherin beta 16 | 272674 | 120322 | 279068 | 26, 37, 24 |
| 5 | PCDHB2 | Protocadherin beta 2 | 112852 | 171815 | 113205 | 38, 24, 12 |
| 5 | PCDHB3 | Protocadherin beta 3 | 113205 | 112852 | 081818 | 42, 24, 23 |
| 5 | PCDHB4 | Protocadherin beta 4 | 081818 | 113205 | 113209 | 14, 42, 28 |
| 5 | PCDHB5 | Protocadherin beta 5 | 113209 | 081818 | 113211 | 37, 12, |
| 5 | PCDHB6 | Protocadherin beta 6 | 113211 | 113209 | 113212 | 13, 27, 38 |
| 5 | PCDHB7 | Protocadherin beta 7 | 113212 | 113211 | 120322 | 24, 6, 10 |
| 5 | PCDHB8 | Protocadherin beta 8 | 120322 | 113212 | 272674 | 24, 42, 38 |
| 5 | PCDHB9 | Protocadherin beta 9 | 177839 | 279068 | 120324 | 26, 24, 28 |
| 5 | PCDHGA1 | Protocadherin gamma subfamily A, 1 | 204956 | 120329 | 081853 | |
| 5 | PCDHGA10 | Protocadherin gamma subfamily A, 10 | 253846 | 253305 | 254122 | |
| 5 | PCDHGA11 | Protocadherin gamma subfamily A, 11 | 253873 | 254122 | 253159 | |
| 5 | PCDHGA12 | Protocadherin gamma subfamily A, 12 | 253159 | 253873 | 240184 | |
| 5 | PCDHGA2 | Protocadherin gamma subfamily A, 2 | 081853 | 204956 | 254245 | |
| 5 | PCDHGA3 | Protocadherin gamma subfamily A, 3 | 254245 | 081853 | 254221 | |
| 5 | PCDHGA4 | Protocadherin gamma subfamily A, 4 | 262576 | 254221 | 253910 | |
| 5 | PCDHGA5 | Protocadherin gamma subfamily A, 5 | 253485 | 253910 | 262209 | |
| 5 | PCDHGA6 | Protocadherin gamma subfamily A, 6 | 253731 | 262209 | 253537 | |
| 5 | PCDHGA7 | Protocadherin gamma subfamily A, 7 | 253537 | 253731 | 253953 | |
| 5 | PCDHGA8 | Protocadherin gamma subfamily A, 8 | 253767 | 253953 | 276547 | 8, 35, 18 |
| 5 | PCDHGA9 | Protocadherin gamma subfamily A, 9 | 261934 | 276547 | 253305 | |
| 5 | PCDHGB1 | Protocadherin gamma subfamily B, 1 | 254221 | 254245 | 262576 | |
| 5 | PCDHGB2 | Protocadherin gamma subfamily B, 2 | 253910 | 262576 | 253485 | |
| 5 | PCDHGB3 | Protocadherin gamma subfamily B, 3 | 262209 | 253485 | 253731 | |
| 5 | PCDHGB4 | Protocadherin gamma subfamily B, 4 | 253953 | 253537 | 253767 | |
| 5 | PCDHGB5 | Protocadherin gamma subfamily B, 5 | 276547 | 253767 | 261934 | |
| 5 | PCDHGB6 | Protocadherin gamma subfamily B, 6 | 253305 | 261934 | 253846 | |
| 5 | PCDHGB7 | Protocadherin gamma subfamily B, 7 | 254122 | 253846 | 253873 | |
| 5 | PCDHGC3 | Protocadherin gamma subfamily C, 3 | 240184 | 253159 | 242419 | 8, 35, 18 |
| 5 | PCDHGC4 | Protocadherin gamma subfamily C, 4 | 242419 | 240184 | 240764 | |
| 5 | PCDHGC5 | Protocadherin gamma subfamily C, 5 | 240764 | 242419 | 131504 | |
| 5 | PCBD2 | pterin-4 alpha-carbinolamine dehydratase 2 | 132570 | 113621 | 152705 | 33, 30, 13 |
| 5 | PURA | purine rich element binding protein A | 185129 | 158458 | 182700 | 29, 31, 28 |
| 5 | PWWP2A | PWWP domain containing 2A | 170234 | 113312 | 170231 | |
| 5 | RAB24 | RAB24, member RAS oncogene family | 169228 | 165671 | 213347 | 37, 5, 28 |
| 5 | RAB3C | RAB3C, member RAS oncogene family | 152932 | 175857 | 113448 | 20, 10, 28 |
| 5 | RAD1 | RAD1 checkpoint DNA exonuclease | 113456 | 205838 | 113460 | |
| 5 | RAD17 | RAD17 checkpoint clamp leader component | 152942 | 273841 | 152939 | 22, 3, 38 |
| 5 | RAD50 | RAD50 double strand break repair protein | 113522 | 113525 | 169194 | 7, 24, 1 |
| 5 | RANBP17 | RAN binding protein 17 | 204764 | 094755 | 275038 | 40, 38, 42 |
| 5 | RANBP3L | RAN binding protein 3 like | 164188 | 152620 | 079215 | 31, 8, 38 |
| 5 | RAPGEF6 | Rap guanine nucleotide exchange factor 6 | 158987 | 158985 | 273217 | 22, 13, 1 |
| 5 | RASA1 | RAS p21 protein activator 1 | 145715 | 127184 | 134480 | 32, 40, 11 |
| 5 | RASGRF2 | Ras protein specific guanine nucleotide releasing factor 2 | 113319 | 113318 | 131730 | 14, 31, 29 |
| 5 | RASGEF1C | RasGEF domain family member 1C | 146090 | 113269 | 050748 | 38, 14, 23 |
| 5 | REEP2 | receptor accessory protein 2 | 132563 | 120733 | 120738 | 8, 37, 14 |
| 5 | REEP5 | receptor accessory protein 5 | 129625 | 153037 | 212643 | 11, 29, 42 |
| 5 | RACK1 | receptor for activated C kinase 1 | 204628 | 146063 | 183718 | |
| 5 | RGS14 | regulator of G-protein signaling 14 | 169220 | 169223 | 131183 | 3, 37, 7 |
| 5 | RGS7BP | regulator pf G-protein signaling 7 binding protein | 186479 | 164197 | 145642 | 2, 42, 8 |
| 5 | RXFP3 | relaxin/insulin like family peptide receptor 3 | 182631 | 151388 | 164175 | |
| 5 | RELL2 | RELT like 2 | 164620 | 171720 | 197948 | 39, 5, 32 |
| 5 | RGMB | repulsive guidance molecule family member b | 174136 | 058729 | 279232 | 26, 1, 35 |
| 5 | RMND5B | required for meiotic nuclear division 5 homolog B | 145916 | 145911 | 145912 | 33, 34, 38 |
| 5 | RAI14 | retinoic acid induced 14 | 039560 | 082196 | 205838 | 43, 11, 6 |
| 5 | ARHGAP26 | Rho GTPase activating protein 26 | 145819 | 113578 | 113580 | 24, 26, 37 |
| 5 | ARHGEF28 | Rho guanine nucleotide exchange factor 28 | 214944 | 164338 | 171617 | 3, 11, 6 |
| 5 | ARHGEF37 | Rho guanine nucleotide exchange factor 37 | 183111 | 113712 | 155846 | 29, 31, 33 |
| 5 | RHOBTB3 | Rho related BTB domain containing 3 | 164292 | 145757 | 173221 | |
| 5 | ROPN1L | rhophilin associated tail protein 1 like | 145491 | 145495 | 164236 | 38, 20, 42 |
| 5 | RPL26L1 | ribosomal protein L26 like 1 | 037241 | 113719 | 113732 | 7, 3, 30 |
| 5 | RPL37 | ribosomal protein L37 | 145592 | 279126 | 132357 | |
| 5 | RPS14 | ribosomal protein S14 | 164587 | 019582 | 070614 | 12, 41, 43 |
| 5 | RPS23 | ribosomal protein S23 | 186468 | 152348 | 205464 | 11, 12, 7 |
| 5 | RFESD | Rieske Fe-S domain containing | 175449 | 178015 | 145757 | |
| 5 | RNF130 | ring finger protein 130 | 113269 | 197226 | 146090 | 29, 31, 6 |
| 5 | RNF14 | ring finger protein 14 | 013561 | 113555 | 113552 | 2, 29, 23 |
| 5 | RNF145 | ring finger protein 145 | 145860 | 164330 | 164332 | 37, 24, 42 |
| 5 | RNF180 | ring finger protein 180 | 164197 | 178394 | 186479 | 29, 1, 45 |
| 5 | RNF44 | ring finger protein 44 | 146083 | 113194 | 074276 | 21, 4, 12 |
| 5 | RIOK2 | RIO kinase 2 | 058729 | 145721 | 174136 | 26, 22, 42 |
| 5 | RBM22 | RNA binding motif protein 22 | 086589 | 164591 | 132912 | 36, 24, 39 |
| 5 | RBM27 | RNA binding motif protein 27 | 091009 | 275740 | 091010 | 18, 44, 22 |
| 5 | POLR3G | RNA polymerase III subunit G | 113356 | 176055 | 176018 | 26, 19, 21 |
| 5 | RICTOR | RPTOR independent companion of MTOR complex 2 | 164327 | 145623 | 082074 | 31, 22, 7 |
| 5 | RUFY1 | RUN and FYVE domain containing 1 | 176783 | 087116 | 169045 | 22, 27, 2 |
| 5 | S100Z | S100 calcium binding protein Z | 171643 | 164251 | 145708 | 7, 6, 5 |
| 5 | SAP30L | SAP30 like | 164576 | 164574 | 113196 | 22, 34, 27 |
| 5 | SGCD | sarcoglycan delta | 170624 | 226650 | 279646 | 15, 1, 29 |
| 5 | SEC24A | SEC24 homolog A, COPII coat complex component | 113615 | 152700 | 164615 | 11, 6, 32 |
| 5 | SPARC | secreted protein acidic and cysteine rich | 113140 | 086570 | 177556 | 11, 6, 43 |
| 5 | SAR1B | secretion associated Ras related GTPase 1B | 152700 | 043143 | 113615 | 30, 43, 2 |
| 5 | SCGB3A1 | secretoglobin family 3A member 1 | 161055 | 113300 | 037280 | 33, 20, 15 |
| 5 | SCGB3A2 | secretoglobin family 3A member 2 | 164265 | 164266 | 178776 | 20, 18, 5 |
| 5 | SCAMP1 | secretory carrier membrane protein 1 | 085365 | 132842 | 145685 | 4, 42, 31 |
| 5 | SELENOP | selenoprotein P | 250722 | 198865 | 177721 | 19, 1, 36 |
| 5 | SEMA5A | semaphorin 5A | 112902 | 124279 | 169777 | 11, 15, 6 |
| 5 | SEMA6A | semaphorin 6A | 092421 | 145781 | 169570 | 12, 35, 32 |
| 5 | SEPT8 | septin 8 | 164402 | 205089 | 198944 | 8, 26, 40 |
| 5 | SQSTM1 | sequestosome 1 | 161011 | 161013 | 161010 | 23, 35, 27 |
| 5 | SERINC5 | serine incorporator 5 | 164300 | 113296 | 164299 | 11, 2, 33 |
| 5 | SPINK1 | serine peptidase inhibitor, Kazal type 1 | 164266 | 176049 | 164265 | 28, 4, 37 |
| 5 | SPINK13 | serine peptidase inhibitor, Kazal type 13 (putative) | 214510 | 248109 | 145879 | 38, |
| 5 | SPINK14 | serine peptidase inhibitor, Kazal type 14 (putative) | 196800 | 133710 | 178172 | |
| 5 | SPINK5 | serine peptidase inhibitor, Kazal type 5 | 133710 | 178776 | 196800 | 13, 24, 30 |
| 5 | SPINK6 | serine peptidase inhibitor, Kazal type 6 | 178172 | 196800 | 248109 | 10, 19, 9 |
| 5 | SPINK7 | serine peptidase inhibitor, Kazal type 7 (putative) | 145879 | 214510 | 204909 | 18, 30, 13 |
| 5 | SPINK9 | serine peptidase inhibitor, Kazal type 9 | 204909 | 145879 | 145868 | 38, |
| 5 | STK10 | serine/threonine kinase 10 | 072786 | 072803 | 214360 | 43, 22, 1 |
| 5 | STK32A | serine/threonine kinase 32A | 169302 | 156475 | 113657 | 4, 2, 30 |
| 5 | SRFBP1 | serum response factor binding protein 1 | 151304 | 181867 | 113083 | 42, 4, 12 |
| 5 | SETD9 | SET domain containing 9 | 155542 | 095015 | 155545 | 34, 13, 38 |
| 5 | SH3PXD2B | SH3 and PX domains 2B | 174705 | 168246 | 214357 | |
| 5 | SH3TC2 | SH3 domain and tetratricopeptide repeats 2 | 169247 | 169252 | 173210 | 32, 11, 7 |
| 5 | SH3RF2 | SH3 domain containing ring finger 2 | 156463 | 204928 | 173261 | 38, 24, 15 |
| 5 | SHROOM1 | shroom family member 1 | 164403 | 198944 | 164404 | 18, 7, 16 |
| 5 | SFXN1 | sideroflexin 1 | 164466 | 184845 | 113749 | 13, 23, 29 |
| 5 | SRP19 | signal recognition particle 19 | 153037 | 272869 | 129625 | 26, 33, 23 |
| 5 | SIL1 | SIL1 nucleotide exchange factor | 120725 | 146006 | 280987 | |
| 5 | SSBP2 | single stranded DNA binding protein 2 | 145687 | 172497 | 152348 | 29, 31, 30 |
| 5 | SKIV2L2 | Ski2 like RNA helicase 2 | 039123 | 067248 | 067113 | 42, 38, 24 |
| 5 | SLIT3 | slit guidance ligand 3 | 184347 | 120137 | 040275 | 29, 1, 15 |
| 5 | SLU7 | SLU7 homolog, splicing factor | 164609 | 221886 | 164611 | 41, 30, 31 |
| 5 | SMAD5 | SMAD family member 5 | 113658 | 120708 | 271824 | 25, 29, 22 |
| 5 | SERF1A | small EDRK-rich factor 1A | 172058 | 205571 | 172062 | |
| 5 | SERF1B | small EDRK-rich factor 1B | 205572 | 183474 | 205571 | |
| 5 | SGTB | small glutamine rich tetratricopeptide repeat containing beta | 197860 | 253251 | 123213 | 26, 34, 8 |
| 5 | SMIM15 | small integral membrane protein 15 | 188725 | 164182 | 130449 | 4, 26, 29 |
| 5 | SMIM23 | small integral membrane protein 23 | 185662 | 156427 | 072803 | |
| 5 | SMIM3 | small integral membrane protein 3 | 256235 | 132912 | 237693 | 1, 29, 35 |
| 5 | SLF1 | SMC5-SMC6 complex localization factor 1 | 133302 | 185261 | 175471 | |
| 5 | SLC1A3 | solute carrier family 1 member 3 | 079215 | 164188 | 164190 | 8, 13, 34 |
| 5 | SLC12A2 | solute carrier family 12 member 2 | 064651 | 230561 | 138829 | 11, 42, 16 |
| 5 | SLC12A7 | solute carrier family 12 member 7 | 113504 | 145506 | 174358 | 29, 1, 37 |
| 5 | SLC22A4 | solute carrier family 22 member 4 | 197208 | 131435 | 197375 | 45, 5, 42 |
| 5 | SLC22A5 | solute carrier family 22 member 5 | 197375 | 197208 | 197536 | 35, 11, 9 |
| 5 | SLC23A1 | solute carrier family 23 member 1 | 170482 | 120727 | 170476 | |
| 5 | SLC25A2 | solute carrier family 25 member 2 | 120329 | 178913 | 204956 | 38, |
| 5 | SLC25A46 | solute carrier family 25 member 46 | 164209 | 186952 | 145777 | 7, 2, 25 |
| 5 | SLC25A48 | solute carrier family 25 member 48 | 145832 | 250378 | 145839 | 17, 8, 14 |
| 5 | SLC26A2 | solute carrier family 26 member 2 | 155850 | 132915 | 164296 | 42, 29, 30 |
| 5 | SLC27A6 | solute carrier family 27 member 6 | 113396 | 138829 | 066583 | 15, 30, 23 |
| 5 | SLC30A5 | solute carrier family 30 member 5 | 145740 | 145675 | 134057 | 45, 4, 11 |
| 5 | SLC34A1 | solute carrier family 34 member 1 | 131183 | 169220 | 196570 | 17, 35, 19 |
| 5 | SLC35A4 | solute carrier family 35 member A4 | 176087 | 113108 | 170458 | 30, 41, 9 |
| 5 | SLC36A1 | solute carrier family 36 member 1 | 123643 | 186335 | 086570 | 26, 29, 18 |
| 5 | SLC36A2 | solute carrier family 36 member 2 | 186335 | 186334 | 123643 | 17, 15, 38 |
| 5 | SLC36A3 | solute carrier family 36 member 3 | 186334 | 196743 | 186335 | 38, 10, |
| 5 | SLC38A9 | solute carrier family 38 member 9 | 177058 | 067113 | 152670 | 29, 38, 2 |
| 5 | SLC4A9 | solute carrier family 4 member 9 | 113073 | 113070 | 131503 | 17, 38, 20 |
| 5 | SLC45A2 | solute carrier family 45 member 2 | 164175 | 182631 | 242110 | 35, 44, 17 |
| 5 | SLC6A18 | solute carrier family 6 member 18 | 164363 | 174358 | 164362 | 17, 16, 38 |
| 5 | SLC6A19 | solute carrier family 6 member 19 | 174358 | 113504 | 164363 | 17, 37, 27 |
| 5 | SLC6A3 | solute carrier family 6 member 3 | 142319 | 049656 | 153395 | 17, 39, 8 |
| 5 | SLC6A7 | solute carrier family 6 member 7 | 011083 | 113722 | 070808 | 8, 16, 14 |
| 5 | SLC9A3 | solute carrier family 9 member A3 | 066230 | 180104 | 112877 | 16, 42, 35 |
| 5 | SLCO4C1 | solute carrier organic anion transporter family member 4C1 | 173930 | 113532 | 205359 | 17, 42, 5 |
| 5 | SLCO6A1 | solute carrier organic anion transporter family member 6A1 | 205359 | 173930 | 145730 | 38, 8, |
| 5 | SNX18 | sorting nexin 18 | 178996 | 169271 | 164283 | 6, 11, 2 |
| 5 | SNX2 | sorting nexin 2 | 205302 | 064692 | 064652 | 30, 36, 18 |
| 5 | SNX24 | sorting nexin 24 | 064652 | 205302 | 168938 | 34, 11, 29 |
| 5 | SOWAHA | sosondowah ankyrin repeat domain family member A | 198944 | 164402 | 164403 | 17, 33, 16 |
| 5 | SPOCK1 | SPARC/osteonectin, cwcv and kazal like domains proteoglycan 1 | 152377 | 069018 | 146021 | |
| 5 | SPEF2 | sperm flagellar 2 | 152582 | 113494 | 168685 | 44, 4, 6 |
| 5 | SPATA24 | spermatogenesis associated 24 | 170469 | 228672 | 170464 | 21, 40, 27 |
| 5 | SPATA9 | spermatogenesis associated 9 | 145757 | 175449 | 164292 | 38, 6, 32 |
| 5 | SPZ1 | spermatogenic leucine zipper 1 | 164299 | 164300 | 039319 | 38, 8, |
| 5 | SKP1 | S-phase kinase associated protein 1 | 113558 | 081059 | 272772 | 33, 20, 38 |
| 5 | SKP2 | S-phase kinase associated protein 2 | 145604 | 164187 | 152620 | 32, 4, 24 |
| 5 | SPDL1 | spindle apparatus coiled-coil protein 1 | 040275 | 184347 | 134516 | 6, 45, 9 |
| 5 | SREK1 | splicing regulatory glutamic acid and lysine rich protein 1 | 153914 | 112851 | 069020 | 7, 1, 2 |
| 5 | SPRY4 | sprouty RTK signaling antagonist 4 | 187678 | 131507 | 113578 | 25, |
| 5 | SREK1IP1 | SREK1 interacting protein 1 | 153006 | 145642 | 153015 | 22, 31, 25 |
| 5 | SOX30 | SRY-box30 | 039600 | 172548 | 187658 | 38, 8, 32 |
| 5 | ST8SIA4 | ST8 alpha-N-acetyl-neuraminide alpha-2,8-sialyltransferase 4 | 113532 | 174132 | 173930 | 42, 22, 5 |
| 5 | STC2 | stanniocalcin 2 | 113739 | 183072 | 145919 | 23, 10, 25 |
| 5 | STARD4 | StAR related lipid transfer domain containing 4 | 164211 | 152495 | 134986 | 26, 45, 7 |
| 5 | SRD5A1 | steroid 5 alpha-reductase 1 | 145545 | 037474 | 112941 | 9, 24, 13 |
| 5 | SRA1 | steroid receptor RNA activator 1 | 213523 | 254996 | 243056 | 34, 21, 26 |
| 5 | SUB1 | SUB1 homolog, transcriptional regulator | 113387 | 056097 | 113389 | 34, 11, 24 |
| 5 | SDHA | succinate dehydrogenase complex flavoprotein subunit A | 073578 | 164366 | 249915 | 15, 24, 3 |
| 5 | SIMC1 | SLIMO interacting motifs containing 1 | 170085 | 182230 | 122203 | 31, 27, 22 |
| 5 | SMN1 | survival of motor neuron 1, telomeric | 172062 | 172058 | 249437 | 37, 24, 43 |
| 5 | SMN2 | survival of motor neuron 2, centromeric | 205571 | 205572 | 172058 | 11, 37, 31 |
| 5 | SV2C | synaptic vesicle glycoprotein 2C | 122012 | 152359 | 254893 | 26, 14, 10 |
| 5 | SYNPO | synaptopodin | 171992 | 070614 | 164591 | 1, 26, 25 |
| 5 | SNCAIP | synuclein alpha interacting protein | 064692 | 250803 | 205302 | |
| 5 | SNCB | synuclein beta | 074317 | 169258 | 175766 | 14, 26, 8 |
| 5 | TAS2R1 | taste 2 receptor member 1 | 169777 | 112902 | 150756 | 10, |
| 5 | TAF7 | TATA-box binding protein associated factor 7 | 178913 | 113248 | 120329 | 36, 39, 42 |
| 5 | TAF9 | TATA-box binding protein associated factor 9 | 273841 | 085231 | 152942 | 44, 43, 34 |
| 5 | TBC1D9B | TBC1 domain family member 9B | 197226 | 161010 | 113269 | 9, 34, 37 |
| 5 | TIMD4 | T-cell immunoglobulin and mucin domain containing 4 | 145850 | 231989 | 113249 | 38, 39, 10 |
| 5 | TLX3 | T-cell leukemia homeobox 3 | 164438 | 275038 | 181163 | 7, 25, |
| 5 | TERT | telomerase reverse transcriptase | 164362 | 164363 | 049656 | 21, 22, 3 |
| 5 | TENM2 | teneurin transmembrane protein 2 | 145934 | 038274 | 113645 | 18, 25, 6 |
| 5 | TEX43 | testis expressed 43 | 196900 | 164902 | 113368 | 38, 8, |
| 5 | TSSK1B | testis specific serine kinase 1B | 212122 | 171444 | 047188 | 38, 8, |
| 5 | TSPAN17 | tetraspanin 17 | 048140 | 175766 | 113763 | 24, 6, 9 |
| 5 | TTC1 | tetratricopeptide repeat domain 1 | 113312 | 170214 | 170234 | 24, 37, 5 |
| 5 | TTC23L | tetratricopeptide repeat domain 23 like | 205838 | 039560 | 113456 | 38, 30, 27 |
| 5 | TTC33 | tetratricopeptide repeat domain 33 | 113638 | 153071 | 171522 | 34, 26, 8 |
| 5 | TTC37 | tetratricopeptide repeat domain 37 | 198677 | 153347 | 164291 | 42, 6, 45 |
| 5 | TXNDC15 | thioredoxin domain containing 15 | 113621 | 181904 | 132570 | 31, 40, 45 |
| 5 | THOC3 | THO complex 3 | 051596 | 145920 | 182230 | |
| 5 | TARS | threonyl-tRNA synthetase | 113407 | 113389 | 151388 | 40, 24, 21 |
| 5 | THBS4 | thrombospondin 4 | 113296 | 177034 | 164300 | 24, 26, 31 |
| 5 | TSLP | thymic stromal lymphopoietin | 145777 | 164209 | 134987 | 34, 40, 10 |
| 5 | TRIP13 | thyroid hormone receptor interactor 13 | 071539 | 028310 | 145506 | 6, 38, 23 |
| 5 | TIFAB | TIFA inhibitor | 255833 | 251380 | 181965 | 2, 22, 44 |
| 5 | TIGD6 | tigger transposable element derived 6 | 164296 | 155850 | 113716 | 36, 24, 10 |
| 5 | TMED7- | TMED7-TICAM2 readthrough | 251201 | 145780 | 243414 | |
| | TICAM2 | | | | | |
| 5 | TNFAIP8 | TNF alpha induced protein 8 | 145779 | 172869 | 133835 | 30, 5, 4 |
| 5 | TNIP1 | TNFAIP3 interacting protein 1 | 145901 | 211445 | 197043 | 21, 34, 36 |
| 5 | TICAM2 | toll like receptor adaptor molecule 2 | 243414 | 251201 | 134970 | 14, |
| 5 | TRAPPC13 | trafficking protein particle complex 13 | 113597 | 113595 | 253251 | 29, 13, 42 |
| 5 | TCERG1 | transcription elongation regulator 1 | 113649 | 091010 | 173250 | 11, 12, 22 |
| 5 | TCF7 | transcription factor 7 (T-cell specific, HMG-box) | 081059 | 213585 | 113558 | 39, 22, 34 |
| 5 | TGFBI | transforming growth factor beta induced | 120708 | 145826 | 113658 | 10, 45, 17 |
| 5 | TRPC7 | transient receptor potential cation channel subfamily | 069018 | 271824 | 152377 | 10, 28, 38 |
| | | C member 7 | | | | |
| 5 | TMCO6 | transmembrane and coiled-coil domains 6 | 113119 | 131495 | 113141 | 31, 9, 21 |
| 5 | TMED7 | transmembrane p24 trafficking protein 7 | 134970 | 243414 | 129596 | 42, 24, 39 |
| 5 | TMED9 | transmembrane p24 trafficking protein 9 | 184840 | 146067 | 027847 | 28, 12, 35 |
| 5 | TMEM161B | transmembrane protein 161B | 164180 | 134480 | 081189 | 18, 36, 22 |
| 5 | TMEM167A | transmembrane protein 167A | 174695 | 205464 | 152422 | 34, 31, 42 |
| 5 | TMEM171 | transmembrane protein 171 | 157111 | 157107 | 164325 | 36, 37, 17 |
| 5 | TMEM173 | transmembrane protein 173 | 184584 | 283288 | 131508 | 7, 5, 36 |
| 5 | TMEM174 | transmembrane protein 174 | 164325 | 157111 | 251493 | 17, 44, |
| 5 | TMEM232 | transmembrane protein 232 | 186952 | 112893 | 164209 | 10, 29, 30 |
| 5 | TMEM267 | transmembrane protein 267 | 151881 | 151882 | 172244 | 1, 4, 31 |
| 5 | TNPO1 | transportin 1 | 083312 | 178175 | 157107 | 45, 2, 39 |
| 5 | TCOF1 | treacle ribosome biogenesis factor 1 | 070814 | 183876 | 019582 | 43, 13, 18 |
| 5 | TRIO | trio Rho guanine nucleotide exchange factor | 038382 | 039139 | 281325 | 31, 22, 6 |
| 5 | TRIM23 | tripartite motif containing 23 | 113595 | 113593 | 113597 | 8, 26, 7 |
| 5 | TRIM36 | tripartite motif containing 36 | 152503 | 080709 | 164219 | 38, 40, 31 |
| 5 | TRIM41 | tripartite motif containing 41 | 146063 | 146054 | 204628 | 39, 26, 28 |
| 5 | TRIM52 | tripartite motif containing 52 | 183718 | 204628 | 230178 | 9, 2, 15 |
| 5 | TRIM7 | tripartite motif containing 7 | 146054 | 182613 | 146063 | 34, 43, 16 |
| 5 | THG1L | tRNA-histidine guanylyltransferase 1 like | 113272 | 187658 | 155858 | 37, 15, 31 |
| 5 | TBCA | tubulin folding cofactor A | 171530 | 171540 | 132842 | |
| 5 | TPPP | tubulin polymerization promoting protein | 171368 | 112877 | 206077 | 29, 8, 18 |
| 5 | UQCRQ | ubiquinol-cytochrome c reductase complex III subunit VII | 164405 | 164404 | 164406 | 15, 29, 7 |
| 5 | UBE2B | ubiquitin conjugating enzyme E2 B | 119048 | 006837 | 237190 | 6, 30, 22 |
| 5 | UBE2D2 | ubiquitin conjugating enzyme E2 D2 | 131508 | 184584 | 171604 | 4, 7, 44 |
| 5 | UBE2QL1 | ubiquitin conjugating enzyme E2 Q family like 1 | 215218 | 133398 | 037474 | 31, 2, 14 |
| 5 | UBTD2 | ubiquitin domain containing 2 | 168246 | 214360 | 174705 | 6, 12, 4 |
| 5 | UIMC1 | ubiquitin interaction motif containing 1 | 087206 | 160883 | 113761 | 21, 9, 37 |
| 5 | UBLCP1 | ubiquitin like domain containing CTD phosphatase 1 | 164332 | 145860 | 113302 | 11, 1, 6 |
| 5 | UGT3A1 | UDP glycosyltransferase family 3 member A1 | 145626 | 152611 | 168671 | 17, 38, 24 |
| 5 | UGT3A2 | UDP glycosyltransferase family 3 member A2 | 168671 | 145626 | 164187 | 39, 38, 18 |
| 5 | UNC5A | unc-5 netrin receptor A | 113763 | 048140 | 160883 | 27, 14, 8 |
| 5 | UTP15 | UTP15, small subunit processome component | 164338 | 164331 | 214944 | 3, 13, 45 |
| 5 | VCAN | versican | 038427 | 152422 | 145681 | 45, 18, 12 |
| 5 | VDAC1 | voltage dependent anion channel 1 | 213585 | 113583 | 081059 | 15, 13, 35 |
| 5 | WDR36 | WD repeat domain 36 | 134987 | 145777 | 152495 | 9, 7, 22 |
| 5 | WDR41 | WD repeat domain 41 | 164253 | 113231 | 171540 | 43, 1, 3 |
| 5 | WDR55 | WD repeat domain 55 | 120314 | 113141 | 256453 | 13, 45, 39 |
| 5 | WDR70 | WD repeat domain 70 | 082068 | 113569 | 168621 | 43, 13, 9 |
| 5 | WNT8A | Wnt family member 8A | 061492 | 031003 | 112981 | |
| 5 | WWC1 | WW and C2 domain containing 1 | 113645 | 145934 | 113643 | 21, 3, 27 |
| 5 | XRCC4 | X-ray repair cross complementing 4 | 152422 | 174695 | 038427 | 10, 7, 5 |
| 5 | YIPF5 | Yip1 domain family member 5 | 145817 | 158497 | 183775 | 45, 26, 43 |
| 5 | YTHDC2 | YTH domain containing 2 | 047188 | 212122 | 080709 | 10, 11, 29 |
| 5 | ZFP2 | ZFP2 zinc finger protein | 198939 | 178338 | 178187 | 23, 38, 17 |
| 5 | ZFP62 | ZFP62 zinc finger protein | 196670 | 131446 | 113303 | 31, 39, 40 |
| 5 | ZBED3 | zinc finger BED-type containing 3 | 132846 | 164252 | 113231 | 7, 17, 30 |
| 5 | ZBED8 | zinc finger BED-type containing 8 | 221886 | 145861 | 164609 | 13, 28, 15 |
| 5 | ZRSR1 | Zinc finger CCCH-type, RNA binding motif and | 212643 | 129625 | 172795 | |
| | | serine/arginine rich 1 | | | | |
| 5 | ZCCHC10 | zinc finger CCHC-type containing 10 | 155329 | 072364 | 170606 | 4, 45, 22 |
| 5 | ZCCHC9 | zinc finger CCHC-type containing 9 | 131732 | 131730 | 172497 | 34, 7, 1 |
| 5 | ZDHHC11 | zinc finger DHHC-type containing 11 | 188818 | 206077 | 028310 | 18, 22, 19 |
| 5 | ZDHHC11B | zinc finger DHHC-type containing 11B | 206077 | 171368 | 188818 | 1, 31, 18 |
| 5 | ZFYVE16 | zinc finger FYVE-type containing 16 | 039319 | 164299 | 152380 | |
| 5 | ZMAT2 | zinc finger matrin-type 2 | 146007 | 112855 | 204970 | 11, 31, 5 |
| 5 | ZNF131 | zinc finger protein 131 | 172262 | 177721 | 177453 | 43, 22, 38 |
| 5 | ZNF300 | zinc finger protein 300 | 145908 | 237693 | 279109 | 8, 25, 12 |
| 5 | ZNF346 | zinc finger protein 346 | 113761 | 087206 | 160867 | 40, 45, 39 |
| 5 | ZNF354A | zinc finger protein 354A | 169131 | 113240 | 178338 | 26, 13, 28 |
| 5 | ZNF354B | zinc finger protein 354B | 178338 | 169131 | 198939 | |
| 5 | ZNF354C | zinc finger protein 354C | 177932 | 234284 | 087116 | 6, 25, 12 |
| 5 | ZNF366 | zinc finger protein 366 | 178175 | 049883 | 083312 | 42, 39, 32 |
| 5 | ZNF454 | zinc finger protein 454 | 178187 | 198939 | 113262 | 37, 38, 23 |
| 5 | ZNF474 | zinc finger protein 474 | 164185 | 113083 | 250803 | 42, 18, 38 |
| 5 | ZNF608 | zinc finger protein 608 | 168916 | 151292 | 155324 | 18, 12, 22 |
| 5 | ZNF622 | zinc finger protein 622 | 173545 | 183654 | 154153 | 18, 9, 6 |
| 5 | ZNF879 | zinc finger protein 879 | 234284 | 113262 | 177932 | 31, 22, 25 |
| 5 | ZFR | zinc finger RNA binding protein | 056097 | 150712 | 113387 | 1, 26, 11 |
| 5 | ZSWIM6 | zinc finger SWIM-type containing 6 | 130449 | 188725 | 068796 | 22, 20, 14 |
| 6 | AGPAT1 | 1-acylglycerol-3-phosphate O-acyltransferase 1 | 204310 | 241404 | 204308 | 2, 35, 9 |
| 6 | AGPAT4 | 1-acylglycerol-3-phosphate O-acyltransferase 4 | 026652 | 085511 | 185345 | 40, 24, 8 |
| 6 | DNPH1 | 2′-deoxynucleoside 5′-phosphate N-hydrolase 1 | 112667 | 112659 | 146216 | 29, 15, 33 |
| 6 | HMGCLL1 | 3-hydroxymethyl-3-methylglutaryl-CoA lyase like 1 | 146151 | 187871 | 112175 | 8, 15, 28 |
| 6 | HTR1B | 5-hydroxytryptamine receptor 1B | 135312 | 112706 | 269964 | 8, |
| 6 | HTR1E | 5-hydroxytryptamine receptor 1E | 168830 | 135316 | 135346 | |
| 6 | NT5DC1 | 5′-nucleotidase domain containing 1 | 178425 | 111816 | 123500 | 29, 22, 2 |
| 6 | NT5E | 5′-nudeotidase ecto | 135318 | 112837 | 135317 | 45, 40, 42 |
| 6 | AHI1 | Abelson helper integration site 1 | 135541 | 118513 | 171408 | 31, 1, 26 |
| 6 | ABHD16A | abhydrolase domain containing 16A | 204427 | 204428 | 204422 | 9, 5, 26 |
| 6 | ABRACL | ABRA C-terminal like | 146386 | 135597 | 112406 | 7, 1, 39 |
| 6 | AIM1 | absent in melanoma 1 | 112297 | 283623 | 130347 | 7, 32, 16 |
| 6 | ACAT2 | acetyl-CoA acetyltransferase 2 | 120437 | 146457 | 120438 | 21, 26, 41 |
| 6 | ARMT1 | acidic residue methyltransferase 1 | 146476 | 155906 | 120262 | 45, 31, 2 |
| 6 | ASCC3 | activating signal cointegrator 1 complex subunit 3 | 112249 | 112246 | 164418 | 29, 26, 1 |
| 6 | ATF6B | activating transcription factor 6 beta | 213676 | 168477 | 204315 | 40, 18, 13 |
| 6 | ABT1 | activator of basal transcription 1 | 146109 | 182952 | 181315 | 3, 22, 31 |
| 6 | ACOT13 | acyl-CoA thioesterase 13 | 112304 | 111802 | 112308 | 3, 29, 10 |
| 6 | ADAT2 | adenosine deaminase, tRNA specific 2 | 189007 | 146416 | 034693 | 22, 40, 29 |
| 6 | AMD1 | adenosylmethionine decarboxylase 1 | 123505 | 155111 | 155115 | 42, 33, 8 |
| 6 | AK9 | adenylate kinase 9 | 155085 | 112365 | 112367 | 31, 23, 42 |
| 6 | ADGRB3 | adhesion G protein-coupled receptor B3 | 135298 | 188107 | 168216 | 8, 31, 2 |
| 6 | ADGRF1 | adhesion G protein-coupled receptor F1 | 153292 | 069122 | 146072 | 42, 24, 17 |
| 6 | ADGRF2 | adhesion G protein-coupled receptor F2 | 164393 | 198087 | 153294 | 20, 35, 8 |
| 6 | ADGRF4 | adhesion G protein-coupled receptor F4 | 153294 | 164393 | 124818 | 24, 45, 13 |
| 6 | ADGRF5 | adhesion G protein-coupled receptor F5 | 069122 | 112818 | 153292 | |
| 6 | ADGRG6 | adhesion G protein-coupled receptor G6 | 112414 | 009844 | 010818 | 32, 43, 26 |
| 6 | AGER | advanced glycosylation end-product specific receptor | 204305 | 204308 | 204304 | 20, 11, 22 |
| 6 | AFDN | afadin, adherens junction formation factor | 130396 | 203690 | 125337 | |
| 6 | AKAP12 | A-kinase anchoring protein 12 | 131016 | 120254 | 181472 | 26, 43, 11 |
| 6 | AKAP7 | A-kinase anchoring protein 7 | 118507 | 079819 | 118520 | 8, 25, 18 |
| 6 | AKIRIN2 | akirin 2 | 135334 | 135336 | 118434 | 39, 22, 41 |
| 6 | AARS2 | alanyl-tRNA synthetase 2, mitochondrial | 124608 | 146221 | 249481 | 21, 23, 12 |
| 6 | ALDH5A1 | aldehyde dehydrogenase 5 family member A1 | 112294 | 112293 | 137261 | 29, 22, 11 |
| 6 | ALDH8A1 | aldehyde dehydrogenase 8 family member A1 | 118514 | 118515 | 112339 | 13, 17, 19 |
| 6 | AIF1 | allograft inflammatory factor 1 | 204472 | 204475 | 204469 | 15, 5, 2 |
| 6 | ATAT1 | alpha tubulin acetyltransferase 1 | 137343 | 204568 | 204564 | 8, 26, 24 |
| 6 | ADTRP | androgen dependent TFPI regulating protein | 111863 | 205269 | 095951 | 43, 37, 16 |
| 6 | AIG1 | androgen induced 1 | 146416 | 010818 | 189007 | 10, 4, 1 |
| 6 | ADGB | androglobin | 118492 | 118508 | 164506 | 10, 42, 30 |
| 6 | ANKS1A | ankyrin repeat and sterile alpha motif domain containing 1A | 064999 | 064995 | 124678 | 36, 26, 35 |
| 6 | ANKRD6 | ankyrin repeat domain 6 | 135299 | 025039 | 083099 | 31, 45, 34 |
| 6 | ANKRD66 | ankyrin repeat domain 66 | 230062 | 146070 | 112818 | 42, 40, 20 |
| 6 | ASF1A | anti-silencing function 1A histone chaperone | 111875 | 111877 | 111879 | 22, 31, 11 |
| 6 | APOBEC2 | apolipoprotein B mRNA editing enzyme catalytic subunit 2 | 124701 | 112212 | 001167 | 25, 15, 26 |
| 6 | APOM | apolipoprotein M | 204444 | 204463 | 204439 | 19, 29, 41 |
| 6 | ARFGEF3 | ARFGEF family member 3 | 112379 | 112378 | 254440 | 28, 33, 29 |
| 6 | ARG1 | arginase 1 | 118520 | 118507 | 112282 | 19, 5, 10 |
| 6 | RARS2 | arginyl-tRNA synthetase 2, mitochondrial | 146282 | 164414 | 135336 | 34, 9, 1 |
| 6 | ARMC12 | armadillo repeat containing 12 | 157343 | 096060 | 196748 | |
| 6 | ARMC2 | armadillo repeat containing 2 | 118690 | 118689 | 080546 | 38, 1, 2 |
| 6 | ATXN1 | ataxin 1 | 124788 | 137198 | 230873 | 34, 43, 31 |
| 6 | ABCC10 | ATP binding cassette subfamily C member 10 | 124574 | 171467 | 171462 | |
| 6 | ABCF1 | ATP binding cassette subfamily F member 1 | 204574 | 204576 | 204569 | 21, 37, 40 |
| 6 | ATP6V1G2- | ATP6V1G2-DDX39B readthrough (NMD candidate) | 254870 | 204511 | 213760 | |
| | DDX39B | | | | | |
| 6 | ATP6V1G2 | ATPase H+ transporting V1 subunit G2 | 213760 | 198563 | 204498 | 26, 8, 22 |
| 6 | ARID1B | AT-rich interaction domain 1B | 049618 | 074771 | 215712 | 40, 11, 9 |
| 6 | ATG5 | autophagy related 5 | 057663 | 085377 | 057657 | 11, 22, 6 |
| 6 | BAK1 | BCL2 antagonist/killer 1 | 030110 | 213588 | 096433 | 21, 5, 44 |
| 6 | BAG2 | BCL2 associated athanogene 2 | 112208 | 112200 | 112210 | 11, 9, 44 |
| 6 | BAG6 | BCL2 associated athanogene 6 | 204463 | 204469 | 204444 | 38, 41, 26 |
| 6 | BCLAF1 | BCL2 associated transcription factor 1 | 029363 | 146410 | 135525 | 22, 45, 24 |
| 6 | BEND3 | BEN domain containing 3 | 178409 | 130349 | 164494 | 20, 27, 28 |
| 6 | BEND6 | BEN domain containing 6 | 151917 | 151914 | 168116 | 24, 2, 8 |
| 6 | B3GALT4 | beta-1,3-galactosyltransferase 4 | 235863 | 231500 | 227057 | 1, 31, 38 |
| 6 | B3GAT2 | beta-1,3-glucuronyltransferase 2 | 112309 | 112305 | 119900 | 14, 8, 45 |
| 6 | BLOC1S5 | biogenesis of lysosomal organelles complex 1 subunit 5 | 188428 | 259040 | 265818 | 22, 37, 24 |
| 6 | BPHL | biphenyl hydrolase like | 137274 | 137275 | 137267 | 17, 27, 31 |
| 6 | BLOC1S5- | BLOC1S5-TXNDC5 readthrough (NMD candidate) | 259040 | 239264 | 188428 | 43, 21, 35 |
| | TXNDC5 | | | | | |
| 6 | BVES | blood vessel epicardial substance | 112276 | 187772 | 132429 | 29, 11, 13 |
| 6 | BMP5 | bone morphogenetic protein 5 | 112175 | 146151 | 124749 | 15, 32, 23 |
| 6 | BMP6 | bone morphogenetic protein 6 | 153162 | 168566 | 239264 | 45, 5, 27 |
| 6 | BCKDHB | branched chain keto acid dehydrogenase E1 subunit beta | 083123 | 112742 | 112773 | 42, 5, 27 |
| 6 | BRPF3 | bromodomain and PHD finger containing 3 | 096070 | 156711 | 180316 | 5, 4, 11 |
| 6 | BRD2 | bromodomain containing 2 | 204256 | 204257 | 204252 | 39, 21, 36 |
| 6 | BACH2 | BTB domain and CNC homolog 2 | 112182 | 135355 | 135341 | 22, 41, 39 |
| 6 | BTBD9 | BTB domain containing 9 | 183826 | 156639 | 124767 | 26, 8, 25 |
| 6 | BTNL2 | butyrophilin like 2 | 204290 | 204296 | 204287 | |
| 6 | BTN1A1 | butyrophilin subfamily 1 member A1 | 124557 | 112763 | 182952 | 22, |
| 6 | BTN2A1 | butyrophilin subfamily 2 member A1 | 112763 | 111801 | 124557 | 26, 39, 30 |
| 6 | BTN2A2 | butyrophilin subfamily 2 member A2 | 124508 | 186470 | 026950 | 36, 42, 39 |
| 6 | BTN3A1 | butyrophilin subfamily 3 member A1 | 026950 | 124508 | 111801 | 42, 36, 39 |
| 6 | BTN3A2 | butyrophilin subfamily 3 member A2 | 186470 | 158406 | 124508 | 36, 39, 30 |
| 6 | BTN3A3 | butyrophilin subfamily 3 member A3 | 111801 | 026950 | 112763 | 36, 39, 37 |
| 6 | BYSL | bystin like | 112578 | 124641 | 112576 | 34, 21, 3 |
| 6 | CAPN11 | calpain 11 | 137225 | 137216 | 262179 | 38, 26, 39 |
| 6 | PKIB | cAMP-dependent protein kinase inhibitor beta | 135549 | 111897 | 164434 | 29, 32, 26 |
| 6 | CAGE1 | cancer antigen 1 | 164304 | 124783 | 124784 | 38, 10, 33 |
| 6 | CNR1 | cannabinoid receptor 1 | 118432 | 118434 | 111880 | 8, 25, 22 |
| 6 | CNPY3 | canopy FGF signaling regulator 3 | 137161 | 171611 | 124713 | 41, 5, 27 |
| 6 | CMTR1 | cap methyltransferase 1 | 137200 | 112130 | 198937 | |
| 6 | CAP2 | CAP, adenylate cyclase-associated protein, 2 (yeast) | 112186 | 112183 | 137414 | 25, 15, 8 |
| 6 | CARMIL1 | capping protein regulator and myosin 1 linker 1 | 079691 | 283467 | 079689 | 29, 16, 20 |
| 6 | CSNK2B | casein kinase 2 beta | 204435 | 204438 | 263020 | 41, 27, 5 |
| 6 | CASP8AP2 | caspase 8 associated protein 2 | 118412 | 112159 | 135355 | 45, 18, 25 |
| 6 | CITED2 | Cbp/p300 interacting transactivator with Glu/Asp rich | 164442 | 164440 | 135577 | 29, 11, 6 |
| | | carboxy-terminal domain 2 | | | | |
| 6 | CCR6 | C-C motif chemokine receptor 6 | 112486 | 213066 | 166984 | 30, 36, 22 |
| 6 | CD109 | CD109 molecule | 156535 | 119899 | 111799 | 29, 13, 45 |
| 6 | CD164 | CD164 molecule | 135535 | 183137 | 185250 | 39, 45, 42 |
| 6 | CD2AP | CD2 associated protein | 198087 | 146072 | 164393 | 4, 16, 22 |
| 6 | CD24 | CD24 molecule | 272398 | 130348 | 130349 | |
| 6 | CD83 | CD83 molecule | 112149 | 180537 | 008083 | 5, 22, 2 |
| 6 | CDKAL1 | CDK5 regulatory subunit associated protein 1 like 1 | 145996 | 112242 | 124766 | 42, 18, 41 |
| 6 | CDC40 | cell division cycle 40 | 168438 | 112290 | 053328 | 42, 23, 9 |
| 6 | CDC5L | cell division cycle 5 like | 096401 | 249481 | 196284 | 43, 7, 34 |
| 6 | CENPQ | centromere protein Q | 031691 | 146085 | 203972 | 31, 37, 21 |
| 6 | CENPW | centromere protein W | 203760 | 066651 | 146374 | 34, 3, 29 |
| 6 | CEP162 | centrosomal protein 162 | 135315 | 135324 | 112837 | 10, 6, 15 |
| 6 | CEP57L1 | centrosomal protein 57 like 1 | 183137 | 080546 | 135535 | 29, 5, 3 |
| 6 | CEP85L | centrosomal protein 85 like | 111860 | 196376 | 198523 | |
| 6 | CLIC1 | chloride intracellular channel 1 | 213719 | 213722 | 204410 | 34, 37, 35 |
| 6 | CLIC5 | chloride intracellular channel 5 | 112782 | 124813 | 001561 | |
| 6 | CDYL | chromodomain Y-like | 153046 | 198721 | 124787 | 26, 9, 29 |
| 6 | C6orf1 | chromosome 6 open reading frame 1 | 186577 | 137309 | 272325 | 26, 21, 7 |
| 6 | C6orf10 | chromosome 6 open reading frame 10 | 204296 | 204301 | 204290 | 38, 14, |
| 6 | C6orf106 | chromosome 6 open reading frame 106 | 196821 | 124664 | 124562 | 1, 9, 14 |
| 6 | C6orf118 | chromosome 6 open reading frame 118 | 112539 | 112531 | 112541 | 42, 38, 20 |
| 6 | C6orf120 | chromosome 6 open reading frame 120 | 185127 | 184465 | 130024 | |
| 6 | C6orf132 | chromosome 6 open reading frame 132 | 188112 | 137413 | 048545 | 3, 23, 16 |
| 6 | C6orf136 | chromosome 6 open reading frame 136 | 204564 | 137343 | 204560 | 5, 29, 25 |
| 6 | C6orf141 | chromosome 6 open reading frame 141 | 197261 | 203972 | 112077 | |
| 6 | C6orf15 | chromosome 6 open reading frame 15 | 204542 | 261272 | 204540 | |
| 6 | C6orf163 | chromosome 6 open reading frame 163 | 203872 | 111850 | 272514 | 36, 17, 23 |
| 6 | C6orf201 | chromosome 6 open reading frame 201 | 185689 | 145975 | 198721 | 38, 19, |
| 6 | C6orf203 | chromosome 6 open reading frame 203 | 130349 | 272398 | 178409 | 31, 10, 39 |
| 6 | C6orf222 | chromosome 6 open reading frame 222 | 189325 | 180316 | 010030 | 4, 3, 16 |
| 6 | C6orf223 | chromosome 6 open reading frame 223 | 181577 | 112715 | 180992 | 9, 26, 21 |
| 6 | C6orf226 | chromosome 6 open reading frame 226 | 221821 | 146223 | 171611 | 34, 6, 20 |
| 6 | C6orf229 | chromosome 6 open reading frame 229 | 260286 | 112312 | 282804 | |
| 6 | C6orf25 | Chromosome 6 open reading frame 25 | 204420 | 244355 | 204421 | 36, 13, 7 |
| 6 | C6orf47 | chromosome 6 open reading frame 47 | 204439 | 204444 | 204438 | 1, 41, 6 |
| 6 | C6orf48 | chromosome 6 open reading frame 48 | 204387 | 204388 | 204386 | |
| 6 | C6orf52 | chromosome 6 open reading frame 52 | 137434 | 111846 | 111845 | 10, 4, 15 |
| 6 | C6orf58 | chromosome 6 open reading frame 58 | 184530 | 214338 | 172673 | 18, 42, 34 |
| 6 | C6orf62 | chromosome 6 open reading frame 62 | 112308 | 112304 | 112312 | 37, 45, 40 |
| 6 | C6orf89 | chromosome 6 open reading frame 89 | 198663 | 137168 | 164530 | 42, 26, 24 |
| 6 | CFAP206 | cilia and flagella associated protein 206 | 272514 | 203872 | 213204 | 29, 38, 42 |
| 6 | CLDN20 | claudin 20 | 171217 | 029639 | 074771 | 35, 16, |
| 6 | CLVS2 | clavesin 2 | 146352 | 172594 | 186439 | 8, 10, 20 |
| 6 | CNKSR3 | CNKSR family member 3 | 153721 | 074706 | 213079 | 11, 23, 17 |
| 6 | F13A1 | coagulation factor XIIIA chain | 124491 | 124785 | 112799 | 42, 32, 1 |
| 6 | COQ3 | coenzyme Q3, methyltransferase | 132423 | 146267 | 132424 | 10, 21, 25 |
| 6 | CCHCR1 | coiled-coil alpha-helical rod protein 1 | 204536 | 204538 | 137310 | 39, 9, 38 |
| 6 | CCDC167 | coiled-coil domain containing 167 | 198937 | 137200 | 112139 | 2, 15, 34 |
| 6 | CCDC170 | coiled-coil domain containing 170 | 120262 | 146476 | 091831 | 36, 30, 40 |
| 6 | CCDC28A | coiled-coil domain containing 28A | 024862 | 279968 | 203734 | 43, 29, 15 |
| 6 | CLPS | colipase | 137392 | 204140 | 197753 | 28, 4, 36 |
| 6 | CLPSL1 | colipase like 1 | 204140 | 196748 | 137392 | 38, |
| 6 | CLPSL2 | colipase like 2 | 196748 | 157343 | 204140 | 31, 38, |
| 6 | COL9A1 | collagen type IX alpha 1 chain | 112280 | 082293 | 082269 | 11, 6, 12 |
| 6 | COL10A1 | collagen type X alpha 1 chain | 123500 | 178425 | 187189 | 6, 18, 28 |
| 6 | COL11A2 | collagen type XI alpha 2 chain | 204248 | 223865 | 204231 | 11, 6, 2 |
| 6 | COL12A1 | collagen type XII alpha 1 chain | 111799 | 156535 | 112695 | 11, 6, 7 |
| 6 | COL19A1 | collagen type XIX alpha 1 chain | 082293 | 168216 | 112280 | 30, 6, 39 |
| 6 | COL21A1 | collagen type XXI alpha 1 chain | 124749 | 112175 | 151914 | 15, 1, 42 |
| 6 | C2 | complement C2 | 166278 | 204371 | 204366 | 19, 36, 10 |
| 6 | C4A | complement C4A (Rodgers blood group) | 244731 | 204344 | 279546 | 19, 26, 1 |
| 6 | C4B | complement C4B (Chido blood group) | 224389 | 279546 | 231852 | 26, 31, 19 |
| 6 | CFB | complement factor B | 243649 | 244255 | 204356 | 19, 4, 44 |
| 6 | CTGF | connective tissue growth factor | 118523 | 197594 | 079931 | 45, 10, 11 |
| 6 | CPNE5 | copine 5 | 124772 | 255587 | 137168 | |
| 6 | CDSN | corneodesmosin | 204539 | 204540 | 204538 | 29, 18, 15 |
| 6 | CTAGE9 | CTAGE family member 9 | 236761 | 180658 | 197594 | 40, 5, 6 |
| 6 | CUL7 | cullin 7 | 044090 | 124541 | 137171 | 43, 27, 7 |
| 6 | CUL9 | cullin 9 | 112659 | 112658 | 112667 | 43, 39, 22 |
| 6 | CUTA | cutA divalent cation tolerance homolog | 112514 | 112511 | 197283 | 29, 30, 31 |
| 6 | CONC | cyclin C | 112237 | 228439 | 112238 | 4, 13, 3 |
| 6 | CCND3 | cyclin D3 | 112576 | 112578 | 137413 | 34, 39, 36 |
| 6 | CDK19 | cyclin dependent kinase 19 | 155111 | 004809 | 123505 | 26, 38, 33 |
| 6 | CDKN1A | cyclin dependent kinase inhibitor 1A | 124762 | 112081 | 255587 | 20, 3, 1 |
| 6 | CRIP3 | cysteine rich protein 3 | 146215 | 137204 | 171467 | 21, 22, 40 |
| 6 | CRISP1 | cysteine rich secretory protein 1 | 124812 | 170950 | 214643 | 38, 33, |
| 6 | CRISP2 | cysteine rich secretory protein 2 | 124490 | 112077 | 096006 | 38, 8, 7 |
| 6 | CRISP3 | cysteine rich secretory protein 3 | 096006 | 124490 | 170950 | 42, 34, 6 |
| 6 | CYB5R4 | cytochrome b5 reductase 4 | 065615 | 203877 | 135324 | 4, 22, 5 |
| 6 | COX7A2 | cytochrome c oxidase subunit 7A2 | 112695 | 111799 | 112697 | 31, 7, 15 |
| 6 | CYP21A2 | cytochrome P450 family 21 subfamily A member 2 | 231852 | 224389 | 168477 | 2, 40, 23 |
| 6 | CYP39A1 | cytochrome P450 family 39 subfamily A member 1 | 146233 | 172348 | 153291 | 10, 29, 26 |
| 6 | DDO | D-aspartate oxidase | 203797 | 053328 | 004809 | 17, 14, 35 |
| 6 | DDX43 | DEAD-box helicase 43 | 080007 | 203907 | 164430 | 24, 38, 30 |
| 6 | DHX16 | DEAH-box helicase 16 | 204560 | 204564 | 146112 | 3, 26, 38 |
| 6 | DAXX | death domain associated protein | 204209 | 236104 | 237649 | 40, 13, 30 |
| 6 | DXO | decapping exoribonudease | 204348 | 204351 | 204344 | |
| 6 | PDSS2 | decaprenyl diphosphate synthase subunit 2 | 164494 | 178409 | 112320 | 43, 42, 24 |
| 6 | DEF6 | DEF6, guanine nucleotide exchange factor | 023892 | 065029 | 112033 | 7, 39, 3 |
| 6 | DEFB110 | defensin beta 110 | 203970 | 214642 | 180872 | 38, |
| 6 | DEFB112 | defensin beta 112 | 180872 | 203970 | 226733 | |
| 6 | DEFB113 | defensin beta 113 | 214642 | 177684 | 203970 | |
| 6 | DEFB114 | defensin beta 114 | 177684 | 214643 | 214642 | |
| 6 | DEFB133 | defensin beta 133 | 214643 | 124812 | 177684 | |
| 6 | DEK | DEK proto-oncogene | 124795 | 165097 | 137393 | 13, 45, 9 |
| 6 | DLL1 | delta like canonical Notch ligand 1 | 198719 | 130023 | 112584 | |
| 6 | DLK2 | delta like non-canonical Notch ligand 2 | 171462 | 124574 | 137221 | 9, 33, 30 |
| 6 | DSE | dermatan sulfate epimerase | 111817 | 187189 | 189241 | 11, 26, 23 |
| 6 | DSP | desmoplakin | 096696 | 124784 | 168566 | 24, 30, 18 |
| 6 | DPPA5 | developmental pluripotency associated 5 | 203909 | 135314 | 203908 | |
| 6 | DDX39B | DExD-box helicase 39B | 198563 | 204511 | 213760 | 39, 44, 12 |
| 6 | DPCR1 | diffuse panbronchiolitis critical region 1 | 168631 | 196260 | 204544 | 37, 28, 2 |
| 6 | DDAH2 | dimethylarginine dimethylaminohydrolase 2 | 213722 | 204421 | 213719 | 41, 12, 29 |
| 6 | DDR1 | discoidin domain receptor tyrosine kinase 1 | 204580 | 137331 | 213780 | |
| 6 | DCBLD1 | discoidin, CUB and LCCL domain containing 1 | 164465 | 282218 | 047932 | |
| 6 | DAAM2 | dishevelled associated activator of morphogenesis 2 | 146122 | 164627 | 124615 | 26, 8, 35 |
| 6 | DACT2 | dishevelled binding antagonist of beta catenin 2 | 164488 | 153303 | 112562 | 32, 44, 14 |
| 6 | POLH | DNA polymerase eta | 170734 | 124571 | 172432 | 21, 22, 45 |
| 6 | DOPEY1 | dopey family member 1 | 083097 | 118420 | 281446 | 40, 31, 23 |
| 6 | DCDC2 | doublecortin domain containing 2 | 146038 | 152954 | 146049 | 1, 31, 19 |
| 6 | DUSP22 | dual specificity phosphatase 22 | 112679 | 172426 | 137265 | 36, 31, 21 |
| 6 | DNAH8 | dynein axonemal heavy chain 8 | 124721 | 124767 | 112164 | 38, 22, 33 |
| 6 | DYNLT1 | dynein light chain Tctex-type 1 | 146425 | 146433 | 164674 | 29, 43, 31 |
| 6 | DST | dystonin | 151914 | 124749 | 151917 | 13, 26, 4 |
| 6 | DTNBP1 | dystrobrevin binding protein 1 | 047579 | 008083 | 007944 | 10, 22, 31 |
| 6 | E2F3 | E2F transcription factor 3 | 112242 | 172197 | 145996 | |
| 6 | ENPP1 | ectonucleotide pyrophosphatase/phosphodiesterase 1 | 197594 | 236761 | 118523 | 7, 29, 11 |
| 6 | ENPP3 | ectonucleotide pyrophosphatase/phosphodiesterase 3 | 154269 | 112282 | 180658 | 42, 39, 10 |
| 6 | ENPP4 | ectonucleotide pyrophosphatase/phosphodiesterase 4 (putative) | 001561 | 112782 | 112796 | 29, 22, 8 |
| 6 | ENPP5 | ectonucleotide pyrophosphatase/phosphodiesterase 5 (putative) | 112796 | 001561 | 172348 | 29, 24, 42 |
| 6 | EEF1E1- | EEF1E1-BLOC1S5 readthrough (NMD candidate) | 265818 | 188428 | 124802 | |
| | BLOC1S5 | | | | | |
| 6 | EFHC1 | EF-hand domain containing 1 | 096093 | 170915 | 065308 | 38, 30, 31 |
| 6 | EGFL8 | EGF like domain multiple 8 | 241404 | 258388 | 204310 | 26, 35, 3 |
| 6 | ELOVL2 | ELOVL fatty acid elongase 2 | 197977 | 153157 | 224531 | 26, 11, 29 |
| 6 | ELOVL4 | ELOVL fatty acid elongase 4 | 118402 | 198478 | 281602 | 39, 45, 8 |
| 6 | ELOVL5 | ELOVL fatty acid elongase 5 | 012660 | 137270 | 001084 | 24, 39, 45 |
| 6 | ERVFRD-1 | Endogenous retrovirus group FRD member 1 | 244476 | 224531 | 111859 | 29, 32, 10 |
| 6 | EDN1 | endothelin 1 | 078401 | 095951 | 112137 | 43, 45, 13 |
| 6 | ECI2 | enoyl-CoA delta isomerase 2 | 198721 | 185689 | 153046 | 43, 33, 25 |
| 6 | EPHA7 | EPH receptor A7 | 135333 | 135341 | 172469 | 29, 11, 12 |
| 6 | ECT2L | epithelial cell transforming 2 like | 203734 | 024862 | 135597 | 42, 20, 14 |
| 6 | EPM2A | EPM2A, laforin glucan phosphatase | 112425 | 152818 | 280184 | 31, 22, 20 |
| 6 | ERMARD | ER membrane associated RNA degradation | 130023 | 184786 | 198719 | 40, 42, 3 |
| 6 | EPB41L2 | erythrocyte membrane protein band 4.1 like 2 | 079819 | 256162 | 118507 | 15, 42, 25 |
| 6 | ESR1 | estrogen receptor 1 | 091831 | 120262 | 131018 | 44, 27, 25 |
| 6 | ECHDC1 | ethylmalonyl-CoA decarboxylase 1 | 093144 | 118518 | 255330 | 11, 13, 22 |
| 6 | ETV7 | ETS variant 7 | 010030 | 189325 | 179165 | 2, 39, 16 |
| 6 | EHMT2 | euchromatic histone lysine methyltransferase 2 | 204371 | 204385 | 166278 | 12, 29, 26 |
| 6 | EEF1A1 | eukaryotic translation elongation factor 1 alpha 1 | 156508 | 135297 | 119899 | 21, 12, 9 |
| 6 | EEF1E1 | eukaryotic translation elongation factor 1 epsilon 1 | 124802 | 265818 | 124786 | 10, 34, 4 |
| 6 | EXOC2 | exocyst complex component 2 | 112685 | 137265 | 188996 | |
| 6 | XPO5 | exportin 5 | 124571 | 137207 | 170734 | 24, 3, 21 |
| 6 | EYA4 | EYA transcriptional coactivator and phosphatase 4 | 112319 | 112306 | 118526 | 11, 25, 39 |
| 6 | EYS | eyes shut homolog (Drosophila) | 188107 | 118482 | 135298 | |
| 6 | EZR | ezrin | 092820 | 164674 | 130363 | 3, 16, 1 |
| 6 | FAXC | failed axon connections homolog | 146267 | 112234 | 132423 | 10, 11, 31 |
| 6 | FAM120B | family with sequence similarity 120B | 112584 | 198719 | 008018 | 13, 24, 10 |
| 6 | FAM135A | family with sequence similarity 135 member A | 082269 | 112280 | 154079 | 18, 25, 26 |
| 6 | FAM162B | family with sequence similarity 162 member B | 183807 | 196911 | 173612 | 30, 32, 2 |
| 6 | FAM184A | family with sequence similarity 184 member A | 111879 | 111875 | 111885 | 22, 1, 32 |
| 6 | FAM217A | family with sequence similarity 217 member A | 145975 | 112739 | 185689 | |
| 6 | FAM229B | family with sequence similarity 229 member B | 203778 | 074935 | 112769 | 31, 38, 2 |
| 6 | FAM26D | family with sequence similarity 26 member D | 164451 | 178033 | 111832 | 32, |
| 6 | FAM26E | family with sequence similarity 26 member E | 178033 | 173626 | 164451 | 45, 43, 32 |
| 6 | FAM26F | family with sequence similarity 26 member F | 188820 | 189241 | 173626 | 36, 20, 10 |
| 6 | FAM46A | family with sequence similarity 46 member A | 112773 | 083123 | 005700 | |
| 6 | FAM50B | family with sequence similarity 50 member B | 145945 | 168994 | 112739 | 9, 6, 22 |
| 6 | FAM65B | family with sequence similarity 65 member B | 111913 | 282804 | 283467 | 5, 36, 22 |
| 6 | FAM8A1 | family with sequence similarity 8 member A1 | 137414 | 112186 | 124789 | 37, 1, 24 |
| 6 | FAM83B | family with sequence similarity 83 member B | 168143 | 137251 | 137252 | 24, 23, 4 |
| 6 | FANCE | Fanconi anemia complementation group E | 112039 | 112033 | 198755 | 18, 32, 5 |
| 6 | FABP7 | fatty acid binding protein 7 | 164434 | 135549 | 172594 | 8, 14, 10 |
| 6 | FBXL4 | F-box and leucine rich repeat protein 4 | 112234 | 184486 | 146267 | 9, 7, 22 |
| 6 | FBXO30 | F-box protein 30 | 118496 | 280184 | 146414 | 43, 22, 44 |
| 6 | FBXO5 | F-box protein 5 | 112029 | 146469 | 112031 | 13, 22, 12 |
| 6 | FBXO9 | F-box protein 9 | 112146 | 112144 | 137270 | 24, 29, 28 |
| 6 | FRMD1 | FERM domain containing 1 | 153303 | 125337 | 164488 | 39, 36, 17 |
| 6 | FGFR10P | FGFR1 oncogene partner | 213066 | 026297 | 112486 | 38, 41, 42 |
| 6 | FRS3 | fibroblast growth factor receptor substrate 3 | 137218 | 096088 | 124593 | 3, 6, 8 |
| 6 | FNDC1 | fibronectin type III domain containing 1 | 164694 | 164691 | 112096 | 6, 15, 26 |
| 6 | FIG4 | FIG4 phosphoinositide 5-phosphatase | 112367 | 155085 | 146360 | 41, 39, 10 |
| 6 | FILIP1 | filamin A interacting protein 1 | 118407 | 112697 | 112701 | 11, 1, 15 |
| 6 | FKBP1C | FK506 binding protein 1C | 198225 | 112232 | 146166 | |
| 6 | FKBP5 | FK506 binding protein 5 | 096060 | 112041 | 157343 | 39, 42, 5 |
| 6 | FKBPL | FK506 binding protein like | 204315 | 213676 | 204314 | 29, 18, 6 |
| 6 | FLOT1 | flotillin 1 | 137312 | 196230 | 137331 | 34, 18, 29 |
| 6 | FOXC1 | forkhead box C1 | 054598 | 137273 | 112699 | 11, 10, 26 |
| 6 | FOXF2 | forkhead box F2 | 137273 | 164379 | 054598 | 9, 24, 37 |
| 6 | FOXO3 | forkhead box O3 | 118689 | 135537 | 118690 | |
| 6 | FOXP4 | forkhead box P4 | 137166 | 096264 | 112559 | 21, 37, 20 |
| 6 | FOXQ1 | forkhead box Q1 | 164379 | 188996 | 137273 | 4, 37, 33 |
| 6 | FHL5 | four and a half LIM domains 5 | 112214 | 014123 | 112218 | 38, 42, 26 |
| 6 | FUCA2 | fucosidase, alpha-L-2, plasma | 001036 | 034693 | 112419 | 3, 37, 32 |
| 6 | FUT9 | fucosyltransferase 9 | 172461 | 172469 | 014123 | 8, 14, 25 |
| 6 | FYN | FYN proto-oncogene, Src family tyrosine kinase | 010810 | 056972 | 112761 | 40, 1, 11 |
| 6 | FRK | fyn related Src family tyrosine kinase | 111816 | 249853 | 178425 | 4, 3, 18 |
| 6 | FGD2 | FYVE, RhoGEF and PH domain containing 2 | 146192 | 137409 | 137193 | 36, 1, 41 |
| 6 | GNL1 | G protein nucleolar 1 (putative) | 204590 | 204592 | 204576 | 39, 13, 3 |
| 6 | GPR31 | G protein-coupled receptor 31 | 120436 | 166984 | 112494 | |
| 6 | GPR6 | G protein-coupled receptor 6 | 146360 | 112367 | 112290 | 8, |
| 6 | GPR63 | G protein-coupled receptor 63 | 112218 | 112214 | 123545 | 18, 2, 17 |
| 6 | GPRC6A | G protein-coupled receptor class C group 6 member A | 173612 | 183807 | 185002 | 10, 17, |
| 6 | GABRR1 | gamma-aminobutyric acid type A receptor rho 1 subunit | 146276 | 146281 | 111886 | 20, 14, 10 |
| 6 | GABRR2 | gamma-aminobutyric acid type A receptor rho2 subunit | 111886 | 146276 | 198833 | 39, 37, 25 |
| 6 | GABBR1 | gamma-aminobutyric acid type B receptor subunit 1 | 204681 | 213886 | 204657 | 8, 26, 4 |
| 6 | GJA1 | gap junction protein alpha 1 | 152661 | 146350 | 025156 | 45, 24, 12 |
| 6 | GJA10 | gap junction protein alpha 10 | 135355 | 118412 | 112182 | |
| 6 | GJB7 | gap junction protein beta 7 | 164411 | 188994 | 111850 | 24, 42, 32 |
| 6 | GJE1 | Gap junction protein epsilon 1 | 203733 | 135577 | 009844 | |
| 6 | GFRAL | GDNF family receptor alpha like | 187871 | 137252 | 146151 | |
| 6 | GMDS | GDP-mannose 4,6-dehydratase | 112699 | 054598 | 145949 | 34, 16, 18 |
| 6 | GMNN | geminin, DNA replication inhibitor | 112312 | 112308 | 260286 | 34, 4, 7 |
| 6 | GTF2H4 | general transcription factor IIH subunit 4 | 213780 | 204580 | 137411 | 34, 21, 35 |
| 6 | GTF2H5 | general transcription factor IIH subunit 5 | 272047 | 122335 | 130338 | 10, 2, 33 |
| 6 | GTF3C6 | general transcription factor IIIC subunit 6 | 155115 | 123505 | 197498 | 41, 33, 21 |
| 6 | GCM1 | glial cells missing homolog 1 | 137270 | 112146 | 012660 | 32, 24, 17 |
| 6 | GCM2 | glial cells missing homolog 2 | 124827 | 111837 | 153157 | 29, |
| 6 | GLTSCR1L | GLTSCR1 like | 112624 | 124659 | 146223 | 22, 11, 7 |
| 6 | GLP1R | glucagon like peptide 1 receptor | 112164 | 124721 | 112167 | 35, 17, |
| 6 | GCNT2 | glucosaminyl (N-acetyl) transferase 2, I-branching enzyme | 111846 | 137203 | 137434 | 42, 24, 25 |
| | | (I blood group) | | | | |
| 6 | GFOD1 | glucose-fructose oxidoreductase domain containing 1 | 145990 | 145979 | 124523 | 39, 13, 15 |
| 6 | GRIK2 | glutamate ionotropic receptor kainate type subunit 2 | 164418 | 112249 | 085382 | 26, 11, 17 |
| 6 | GRM1 | glutamate metabotropic receptor 1 | 152822 | 146414 | 118508 | 8, 25, 14 |
| 6 | GRM4 | glutamate metabotropic receptor 4 | 124493 | 096395 | 137309 | 8, 28, 20 |
| 6 | GCLC | glutamate-cysteine ligase catalytic subunit | 001084 | 012660 | 124743 | 24, 23, 42 |
| 6 | QRSL1 | glutaminyl-tRNA synthase (glutamine-hydrolyzing)-like 1 | 130348 | 130347 | 272398 | 10, 45, 4 |
| 6 | GPX5 | glutathione peroxidase 5 | 224586 | 198704 | 232040 | 38, |
| 6 | GPX6 | glutathione peroxidase 6 | 198704 | 187987 | 224586 | |
| 6 | GSTA1 | glutathione S-transferase alpha 1 | 243955 | 244067 | 182793 | 2, 30, 19 |
| 6 | GSTA2 | glutathione S-transferase alpha 2 | 244067 | 096092 | 243955 | 19, 38, 17 |
| 6 | GSTA3 | glutathione S-transferase alpha 3 | 174156 | 182793 | 170899 | 2, 29, 32 |
| 6 | GSTA4 | glutathione S-transferase alpha 4 | 170899 | 174156 | 112144 | 29, 2, 15 |
| 6 | GSTA5 | glutathione S-transferase alpha 5 | 182793 | 243955 | 174156 | |
| 6 | GNMT | glycine N-methyltransferase | 124713 | 137161 | 124587 | 19, 36, 33 |
| 6 | GLYATL3 | glycine-N-acyltransferase like 3 | 203972 | 031691 | 197261 | 44, |
| 6 | CGA | glycoprotein hormones, alpha polypeptide | 135346 | 168830 | 188994 | 31, 32, 11 |
| 6 | GINM1 | glycoprotein integral membrane 1 | 055211 | 131013 | 186625 | 1, 40, 17 |
| 6 | GPLD1 | glycosylphosphatidylinositol specific phospholipase D1 | 112293 | 124532 | 112294 | 21, 19, 22 |
| 6 | GLO1 | glyoxalase I | 124767 | 183826 | 124721 | 13, 45, 43 |
| 6 | GOPC | golgi associated PDZ and coiled-coil motif containing | 047932 | 164465 | 153989 | 11, 22, 1 |
| 6 | GPANK1 | G-patch domain and ankyrin repeats 1 | 204438 | 204439 | 204435 | 29, 9, 31 |
| 6 | GPSM3 | G-protein signaling modulator 3 | 213654 | 204304 | 204301 | 41, 5, 36 |
| 6 | GTPBP2 | GTP binding protein 2 | 172432 | 170734 | 124688 | 9, 24, 29 |
| 6 | GMPR | guanosine monophosphate reductase | 137198 | 007944 | 124788 | 3, 18, 34 |
| 6 | GUCA1A | guanylate cyclase activator 1A | 048545 | 188112 | 214732 | 14, 38, 12 |
| 6 | GUCA1B | guanylate cyclase activator 1B | 112599 | 214732 | 048544 | 14, 25, 10 |
| 6 | GVQW2 | GVQW motif containing 2 | 279968 | 135540 | 024862 | 32, 23, 22 |
| 6 | HBS1L | HBS1 like translational GTPase | 112339 | 118514 | 118513 | 39, 13, 43 |
| 6 | HDDC2 | HD domain containing 2 | 111906 | 111907 | 135547 | |
| 6 | HECA | hdc homolog, cell cycle regulator | 112406 | 146386 | 164440 | 29, 1, 22 |
| 6 | HSP90AB1 | heat shock protein 90 alpha family class B member 1 | 096384 | 112759 | 157593 | 7, 21, 35 |
| 6 | HSPA1L | heat shock protein family A (Hsp70) member 1 like | 204390 | 204392 | 204389 | 38, 31, 10 |
| 6 | HSPA1A | heat shock protein family A (Hsp70) member 1A | 204389 | 204390 | 204388 | 36, 13, 42 |
| 6 | HSPA1B | heat shock protein family A (Hsp70) member 1B | 204388 | 204389 | 204387 | 13, 36, 4 |
| 6 | HSF2 | heat shock transcription factor 2 | 025156 | 152661 | 111897 | 34, 11, 10 |
| 6 | HACE1 | HECT domain and ankyrin repeat containing E3 ubiquitin | 085382 | 164418 | 187772 | 31, 42, 39 |
| | | protein ligase 1 | | | | |
| 6 | HEBP2 | heme binding protein 2 | 051620 | 262543 | 135540 | 33, 21, 1 |
| 6 | HFE | hemochromatosis | 010704 | 187837 | 197061 | 36, 4, 42 |
| 6 | HS3ST5 | heparan sulfate-glucosamine 3-sulfotransferase 5 | 249853 | 196591 | 111816 | 10, |
| 6 | HDGFL1 | hepatoma derived growth factor-like 1 | 112273 | 172179 | 152954 | 38, 33, 8 |
| 6 | HEY2 | hes related family bHLH transcription factor with | 135547 | 111906 | 111912 | 45, 18, 2 |
| | | YRPW motif 2 | | | | |
| 6 | HMGA1 | high mobility group AT-hook 1 | 137309 | 124493 | 186577 | |
| 6 | HMGN3 | high mobility group nucleosomal binding domain 3 | 118418 | 146247 | 135338 | 31, 29, 30 |
| 6 | HMGN4 | high mobility group nucleosomal binding domain 4 | 182952 | 124557 | 146109 | 18, 1, 31 |
| 6 | HINT3 | histidine triad nucleotide binding protein 3 | 111911 | 111912 | 066651 | 1, 42, 43 |
| 6 | HIST1H1A | histone cluster 1 H1 family member a | 124610 | 112343 | 275714 | 16, |
| 6 | HIST1H1B | histone cluster 1 H1 family member b | 184357 | 276903 | 275379 | 4, 35, 16 |
| 6 | HIST1H1C | histone cluster 1 H1 family member c | 187837 | 278272 | 010704 | 26, 7, 3 |
| 6 | HIST1H1D | histone cluster 1 H1 family member d | 124575 | 274750 | 274618 | 39, 13, 22 |
| 6 | HIST1H1E | histone cluster 1 H1 family member e | 168298 | 180573 | 158373 | 24, 17, 8 |
| 6 | HIST1H1T | histone cluster 1 H1 family member t | 187475 | 197061 | 180596 | 38, |
| 6 | HIST1H2AA | histone cluster 1 H2A family member a | 164508 | 079689 | 146047 | 38, |
| 6 | HIST1H2AB | histone cluster 1 H2A family member b | 278463 | 274267 | 276410 | 19, 38, |
| 6 | HIST1H2AC | histone cluster 1 H2A family member c | 180573 | 180596 | 168298 | 37, 9, 1 |
| 6 | HIST1H2AD | histone cluster 1 H2A family member d | 196866 | 197409 | 277224 | |
| 6 | HIST1H2AE | histone cluster 1 H2A family member e | 277075 | 273802 | 274750 | 13, 39, 7 |
| 6 | HIST1H2AG | histone cluster 1 H2A family member g | 196787 | 124635 | 276180 | 21, 9, 27 |
| 6 | HIST1H2AH | histone cluster 1 H2A family member h | 274997 | 197903 | 112812 | 7, 45, 21 |
| 6 | HIST1H2AI | histone cluster 1 H2A family member i | 196747 | 185130 | 278828 | 24, 39, 5 |
| 6 | HIST1H2AJ | histone cluster 1 H2A family member j | 276368 | 278828 | 273703 | 14, 10, |
| 6 | HIST1H2AK | histone cluster 1 H2A family member k | 275221 | 233822 | 276903 | 31, 39, 22 |
| 6 | HIST1H2AL | histone cluster 1 H2A family member l | 276903 | 275221 | 184357 | 32, |
| 6 | HIST1H2AM | histone cluster 1 H2A family member m | 278677 | 197153 | 274641 | 39, 14, 24 |
| 6 | HIST1H2BA | histone cluster 1 H2B family member a | 146047 | 164508 | 146039 | 38, |
| 6 | HIST1H2BB | histone cluster 1 H2B family member b | 276410 | 278463 | 278272 | |
| 6 | HIST1H2BC | histone cluster 1 H2B family member c | 180596 | 187475 | 180573 | |
| 6 | HIST1H2BD | histone cluster 1 H2B family member d | 158373 | 168298 | 274290 | 11, 39, 33 |
| 6 | HIST1H2BE | histone cluster 1 H2B family member e | 274290 | 158373 | 277157 | 23, 20, |
| 6 | HIST1H2BF | histone cluster 1 H2B family member f | 277224 | 196866 | 276966 | 45, 17, 33 |
| 6 | HIST1H2BG | histone cluster 1 H2B family member g | 273802 | 276966 | 277075 | 27, 13, 39 |
| 6 | HIST1H2BH | histone cluster 1 H2B family member h | 275713 | 277775 | 273983 | 7, 39, 19 |
| 6 | HIST1H2BI | histone cluster 1 H2B family member i | 278588 | 273983 | 158406 | |
| 6 | HIST1H2BJ | histone cluster 1 H2B family member j | 124635 | 181315 | 196787 | 38, |
| 6 | HIST1H2BK | histone cluster 1 H2B family member k | 197903 | 276180 | 274997 | |
| 6 | HIST1H2BL | histone cluster 1 H2B family member l | 185130 | 096654 | 196747 | 7, 44, |
| 6 | HIST1H2BM | histone cluster 1 H2B family member m | 273703 | 276368 | 197238 | 3, 23, 38 |
| 6 | HIST1H2BN | histone cluster 1 H2B family member n | 233822 | 273542 | 275221 | |
| 6 | HIST1H2BO | histone cluster 1 H2B family member o | 274641 | 278677 | 168131 | 19, |
| 6 | HIST1H3A | histone cluster 1 H3 family member a | 275714 | 124610 | 278637 | 44, 19, |
| 6 | HIST1H3B | histone cluster 1 H3 family member b | 274267 | 278705 | 278463 | 39, 19, 17 |
| 6 | HIST1H3C | histone cluster 1 H3 family member c | 278272 | 276410 | 187837 | 38, |
| 6 | HIST1H3D | histone cluster 1 H3 family member d | 197409 | 282988 | 196866 | 30, 9, 36 |
| 6 | HIST1H3E | histone cluster 1 H3 family member e | 274750 | 277075 | 124575 | |
| 6 | HIST1H3F | histone cluster 1 H3 family member f | 277775 | 275663 | 275713 | 19, 10, 35 |
| 6 | HIST1H3G | histone cluster 1 H3 family member g | 273983 | 275713 | 278588 | 5, 35, 17 |
| 6 | HIST1H3H | histone cluster 1 H3 family member h | 278828 | 196747 | 276368 | 16, 34, 32 |
| 6 | HIST1H3I | histone cluster 1 H3 family member i | 275379 | 184357 | 275126 | 22, 16, 8 |
| 6 | HIST1H3J | histone cluster 1 H3 family member j | 197153 | 275126 | 278677 | |
| 6 | HIST1H4A | histone cluster 1 H4 family member a | 278637 | 275714 | 278705 | 3, |
| 6 | HIST1H4B | histone cluster 1 H4 family member b | 278705 | 278637 | 274267 | 22, 38, 19 |
| 6 | HIST1H4C | histone cluster 1 H4 family member c | 197061 | 010704 | 187475 | 7, 5, 19 |
| 6 | HIST1H4D | histone cluster 1 H4 family member d | 277157 | 274290 | 282988 | 1, 45, |
| 6 | HIST1H4E | histone cluster 1 H4 family member e | 276966 | 277224 | 273802 | 7, 39, 33 |
| 6 | HIST1H4F | histone cluster 1 H4 family member f | 274618 | 124575 | 275663 | 5, 10, |
| 6 | HIST1H4G | histone cluster 1 H4 family member g | 275663 | 274618 | 277775 | |
| 6 | HIST1H4H | histone cluster 1 H4 family member h | 158406 | 278588 | 186470 | 23, 26, 33 |
| 6 | HIST1H4I | histone cluster 1 H4 family member i | 276180 | 196787 | 197903 | 12, 16, |
| 6 | HIST1H4J | histone cluster 1 H4 family member j | 197238 | 273703 | 273542 | 33, 12, 27 |
| 6 | HIST1H4K | histone cluster 1 H4 family member k | 273542 | 197238 | 233822 | 24, 36, 15 |
| 6 | HIST1H4L | histone cluster 1 H4 family member l | 275126 | 275379 | 197153 | |
| 6 | HDAC2 | histone deacetylase 2 | 196591 | 277443 | 249853 | 11, 45, 13 |
| 6 | HCG27 | HLA complex group 27 (non-protein coding) | 206344 | 204531 | 204525 | 5, 40, 7 |
| 6 | HIVEP1 | human immunodeficiency virus type I enhancer binding protein 1 | 095951 | 111863 | 078401 | 29, 31, 22 |
| 6 | HIVEP2 | human immunodeficiency virus type I enhancer binding protein 2 | 010818 | 112414 | 146416 | 18, 22, 25 |
| 6 | HUS1B | HUS1 checkpoint clamp component B | 188996 | 112685 | 164379 | 37, 38, |
| 6 | HSD17B8 | hydroxysteroid 17-beta dehydrogenase 8 | 204228 | 112473 | 204227 | 29, 9, 13 |
| 6 | HCRTR2 | hypecretin receptor 2 | 137252 | 168143 | 187871 | 10, 20, 8 |
| 6 | IER3 | immediate early response 3 | 137331 | 137312 | 204580 | 11, 30, 9 |
| 6 | IBTK | inhibitor of Bruton tyrosine kinase | 005700 | 112773 | 146242 | 1, 9, 29 |
| 6 | ID4 | inhibitor of DNA binding 4, HLH protein | 172201 | 137393 | 172197 | |
| 6 | ITPR3 | inositol 1,4,5-trisphosphate receptor type 3 | 096433 | 030110 | 137288 | 40, 18, 26 |
| 6 | IP6K3 | inositol hexakisphosphate kinase 3 | 161896 | 137288 | 161904 | 24, 15, 19 |
| 6 | IGF2R | insulin like growth factor 2 receptor | 197081 | 130368 | 175003 | 1, 22, 40 |
| 6 | IPCEF1 | interaction protein for cytohesin exchange factors 1 | 074706 | 112038 | 153721 | 5, 24, 8 |
| 6 | IFNGR1 | interferon gamma receptor 1 | 027697 | 164485 | 177468 | 30, 1, 13 |
| 6 | IRF4 | interferon regulatory factor 4 | 137265 | 112679 | 112685 | 35, 41, 5 |
| 6 | IRAK1BP1 | interleukin 1 receptor associated kinase 1 binding protein 1 | 146243 | 269964 | 146247 | 29, 4, 37 |
| 6 | IL17A | interleukin 17A | 112115 | 170927 | 112116 | |
| 6 | IL17F | interleukin 17F | 112116 | 112115 | 112118 | 5, |
| 6 | IL20RA | interleukin 20 receptor subunit alpha | 016402 | 182747 | 164485 | 43, 31, 16 |
| 6 | IL22RA2 | interleukin 22 receptor subunit alpha 2 | 164485 | 016402 | 027697 | 20, 32, 38 |
| 6 | IMPG1 | interphotoreceptor matrix proteoglycan 1 | 112706 | 196586 | 135312 | 14, 43, 25 |
| 6 | ICK | intestinal cell kinase | 112144 | 170899 | 112146 | 42, 11, 22 |
| 6 | IYD | iodotyrosine deicdinase | 009765 | 198729 | 120278 | 40, 17, 16 |
| 6 | JARID2 | jumonji and AT-rich interaction domain containing 2 | 008083 | 112149 | 047579 | 7, 24, 9 |
| 6 | KPNA5 | karyopherin subunit alpha 5 | 196911 | 153975 | 183807 | 38, 22, 25 |
| 6 | KATNA1 | katanin catalytic subunit A1 | 186625 | 055211 | 131023 | 10, 1, 6 |
| 6 | KLHDC3 | kelch domain containing 3 | 124702 | 124733 | 124541 | 13, 38, 8 |
| 6 | KLHL31 | kelch like family member 31 | 124743 | 001084 | 137269 | 25, 1, 18 |
| 6 | KLHL32 | kelch like family member 32 | 186231 | 123545 | 146263 | 8, 42, 40 |
| 6 | KHDC1L | KH domain containing 1 like | 256980 | 243501 | 135314 | 6, 38, |
| 6 | KHDC3L | KH domain containing 3 like, subcortical maternal | 203908 | 203909 | 203907 | 25, 38, |
| | | complex member | | | | |
| 6 | KHDC1 | KH homology domain containing 1 | 135314 | 256980 | 203909 | |
| 6 | KHDRBS2 | KH RNA binding domain containing, signal transduction | 112232 | 279892 | 198225 | 36, 12, 8 |
| | | associated 2 | | | | |
| 6 | KIAA0319 | KIAA0319 | 137261 | 112294 | 111802 | 13, 3, 8 |
| 6 | KIAA0408 | KIAA0408 | 189367 | 255330 | 214338 | |
| 6 | KIAA1586 | KIAA1586 | 168116 | 151917 | 112200 | 34, 5, 4 |
| 6 | KAAG1 | kidney associated antigen 1 | 146049 | 146038 | 124532 | 31, 4, 19 |
| 6 | KIF13A | kinesin family member 13A | 137177 | 124789 | 187566 | 18, 45, 30 |
| 6 | KIF25 | kinesin family member 25 | 125337 | 130396 | 153303 | 44, 32, |
| 6 | KIF6 | kinesin family member 6 | 164627 | 095981 | 146122 | 30, 10, 38 |
| 6 | KIFC1 | kinesin family member C1 | 237649 | 204209 | 112511 | 21, 39, 3 |
| 6 | KLC4 | kinesin light chain 4 | 137171 | 044090 | 112651 | 42, 26, 16 |
| 6 | L3MBTL3 | l(3)mbt-like 3 (Drosophila) | 198945 | 203756 | 164483 | 18, 16, 39 |
| 6 | LACE1 | lactation elevated 1 | 135537 | 112335 | 118689 | 37, 14, 17 |
| 6 | LAMA2 | laminin subunit alpha 2 | 196569 | 152894 | 146376 | 11, 15, 31 |
| 6 | LAMA4 | laminin subunit alpha 4 | 112769 | 203778 | 281613 | 1, 43, 45 |
| 6 | LATS1 | large tumor suppressor kinase 1 | 131023 | 186625 | 278899 | 1, 11, 4 |
| 6 | LCA5 | LCA5, lebercilin | 135338 | 118418 | 198478 | 45, 34, 42 |
| 6 | LRP11 | LDL receptor related protein 11 | 120256 | 120265 | 164520 | 29, 20, 9 |
| 6 | LEMD2 | LEM domain containing 2 | 161904 | 161896 | 096395 | 26, 43, 27 |
| 6 | LGSN | lengsin, lens protein with glutamine synthetase domain | 146166 | 198225 | 112245 | 14, 27, 44 |
| 6 | LRFN2 | leucine rich repeat and fibronectin type III domain | 156564 | 124615 | 124602 | 8, 3, 36 |
| | | containing 2 | | | | |
| 6 | LRRC1 | leucine rich repeat containing 1 | 137269 | 124743 | 146147 | 32, 4, 26 |
| 6 | LRRC73 | leucine rich repeat containing 73 | 204052 | 137221 | 171453 | 38, 31, 34 |
| 6 | LST1 | leukocyte specific transcript 1 | 204482 | 227507 | 204475 | 7, 29, 5 |
| 6 | LIN28B | lin-28 homolog B | 187772 | 085382 | 112276 | 11, 32, 12 |
| 6 | LHFPL5 | lipoma HMGIC fusion partner-like 5 | 197753 | 137392 | 096063 | 25, 8, |
| 6 | LPA | lipoprotein(a) | 198670 | 146477 | 122194 | 2, 7, 19 |
| 6 | LMBRD1 | LMBR1 domain containing 1 | 168216 | 135298 | 082293 | |
| 6 | LSM2 | LSM2 homolog, U6 small nuclear RNA and mRNA degradation | 204392 | 204394 | 204390 | 41, 15, 14 |
| | | associated | | | | |
| 6 | LTV1 | LTV1 ribosome biogenesis factor | 135521 | 257065 | 280148 | 9, 1, 4 |
| 6 | LY6G5B | lymphocyte antigen 6 complex, locus G5B | 240053 | 263020 | 204428 | 40, 23, 30 |
| 6 | LY6G5C | lymphocyte antigen 6 complex, locus G5C | 204428 | 240053 | 204427 | 28, 36, 39 |
| 6 | LY6G6C | lymphocyte antigen 6 complex, locus G6C | 204421 | 204420 | 213722 | 35, 15, 27 |
| 6 | LY6G6D | lymphocyte antigen 6 complex, locus G6D | 244355 | 255552 | 204420 | 33, |
| 6 | LY6G6E | Lymphocyte antigen 6 complex, locus G6E | 255552 | 250641 | 244355 | 24, 8, |
| 6 | LY6G6F | lymphocyte antigen 6 complex, locus G6F | 204424 | 204422 | 250641 | 36, 16, 19 |
| 6 | LY86 | lymphocyte antigen 86 | 112799 | 124491 | 124782 | 22, 30, 21 |
| 6 | LTA | lymphotoxin alpha | 226979 | 204498 | 232810 | 41, 30, 21 |
| 6 | LTB | lymphotoxin beta | 227507 | 232810 | 204482 | 41, 21, 36 |
| 6 | LYRM2 | LYR motif containing 2 | 083099 | 135299 | 112159 | 11, 31, 40 |
| 6 | LYRM4 | LYR motif containing 4 | 214113 | 219607 | 145982 | 41, 14, 20 |
| 6 | KDM1B | lysine demethylase 1B | 165097 | 137364 | 124795 | 40, 5, 22 |
| 6 | MB21D1 | Mab-21 domain containing 1 | 164430 | 080007 | 135297 | 36, 13, 9 |
| 6 | MAD2L1BP | MAD2L1 binding protein | 124688 | 172432 | 172426 | |
| 6 | MFSD4B | major facilitator superfamily domain containing 4B | 173214 | 112394 | 009413 | |
| 6 | HLA-A | major histocompatibility complex, class I, A | 206503 | 204632 | 066379 | 36, 1, 16 |
| 6 | HLA-B | major histocompatibility complex, class I, B | 234745 | 204525 | 204520 | |
| 6 | HLA-C | major histocompatibility complex, class I, C | 204525 | 206344 | 234745 | |
| 6 | HLA-E | major histocompatibility complex, class I, E | 204592 | 241370 | 204590 | 1, 41, 36 |
| 6 | HLA-F | major histocompatibility complex, class I, F | 204642 | 204644 | 204632 | 36, 22, 41 |
| 6 | HLA-G | major histocompatibility complex, class I, G | 204632 | 204642 | 206503 | 32, 16, 39 |
| 6 | HLA-DMA | major histocompatibility complex, class II, DM alpha | 204257 | 248993 | 204256 | 41, 30, 20 |
| 6 | HLA-DMB | major histocompatibility complex, class II, DM beta | 242574 | 168394 | 248993 | 30, 36, 1 |
| 6 | HLA-DOA | major histocompatibility complex, class II, DO alpha | 204252 | 204256 | 231389 | 22, 7, 21 |
| 6 | HLA-DOB | major histocompatibility complex, class II, DO beta | 241106 | 232629 | 250264 | 41, 21, 22 |
| 6 | HLA-DPA1 | major histocompatibility complex, class II, DP alpha 1 | 231389 | 204252 | 223865 | 41, 30, 21 |
| 6 | HLA-DPB1 | major histocompatibility complex, class II, DP beta 1 | 223865 | 231389 | 204248 | 41, 22, 21 |
| 6 | HLA-DQA1 | major histocompatibility complex, class II, DQ alpha 1 | 196735 | 196126 | 179344 | |
| 6 | HLA-DQA2 | major histocompatibility complex, class II, DQ alpha 2 | 237541 | 179344 | 232629 | |
| 6 | HLA-DQB1 | major histocompatibility complex, class II, DQ beta 1 | 179344 | 196735 | 237541 | 41, 36, 4 |
| 6 | HLA-DQB2 | major histocompatibility complex, class II, DQ beta 2 | 232629 | 237541 | 241106 | 4, 30, 41 |
| 6 | HLA-DRA | major histocompatibility complex, class II, DR alpha | 204287 | 204290 | 198502 | 30, 41, 22 |
| 6 | HLA-DRB1 | major histocompatibility complex, class II, DR beta 1 | 196126 | 198502 | 196735 | |
| 6 | HLA-DRB5 | major histocompatibility complex, class II, DR beta 5 | 198502 | 204287 | 196126 | |
| 6 | MAK | male germ cell associated kinase | 111837 | 272162 | 124827 | 14, 13, 18 |
| 6 | MEA1 | male-enhanced antigen 1 | 124733 | 112640 | 124702 | 33, 27, 31 |
| 6 | ME1 | malic enzyme 1 | 065833 | 013392 | 146250 | 10, 13, 31 |
| 6 | MDGA1 | MAM domain containing glycosylphosphatidylinositol anchor 1 | 112139 | 198937 | 156639 | 24, 8, 14 |
| 6 | MAN1A1 | mannosidase alpha class 1A member 1 | 111885 | 111879 | 146350 | 1, 11, 45 |
| 6 | MANEA | mannosidase endo-alpha | 172469 | 135333 | 172461 | |
| 6 | MAS1L | MAS1 proto-oncogene like, G protein-coupled receptor | 204687 | 204688 | 213886 | |
| 6 | MAS1 | MAS1 proto-oncogene, G protein-coupled receptor | 130368 | 146453 | 197081 | |
| 6 | MED20 | mediator complex subunit 20 | 124641 | 164663 | 112578 | 34, 13, 45 |
| 6 | MED23 | mediator complex subunit 23 | 112282 | 118520 | 154269 | 18, 26, 42 |
| 6 | MDC1 | mediator of DNA damage checkpoint 1 | 137337 | 137404 | 196230 | 43, 29, 40 |
| 6 | MEI4 | meiotic double-stranded break formation protein 4 | 269964 | 135312 | 146243 | |
| 6 | MCHR2 | melanin concentrating hormone receptor 2 | 152034 | 112238 | 112246 | 26, 8, |
| 6 | MRAP2 | melanocortin 2 receptor accessory protein 2 | 135324 | 065615 | 135315 | 34, 16, 17 |
| 6 | MBOAT1 | membrane bound O-acyltransferase domain containing 1 | 172197 | 172201 | 112242 | 18, 11, 24 |
| 6 | MEP1A | meprin A subunit alpha | 112818 | 230062 | 069122 | 16, 37, 3 |
| 6 | MTHFD1L | methylenetetrahydrofolate dehydrogenase (NADP+ | 120254 | 120278 | 131016 | 21, 45, 27 |
| | | dependent) 1-like | | | | |
| 6 | MUT | methylmalonyl-CoA mutase | 146085 | 244694 | 031691 | 29, 24, 19 |
| 6 | METTL24 | methyltransferase like 24 | 053328 | 168438 | 203797 | 27, 12, |
| 6 | MICA | MHC class I polypeptide-related sequence A | 204520 | 234745 | 204516 | 6, 13, 40 |
| 6 | MICB | MHC class I polypeptide-related sequence B | 204516 | 204520 | 204511 | 5, 36, 21 |
| 6 | MICAH | microtubule associated monooxygenase, calponin and LIM | 135596 | 135587 | 112365 | 26, 39, 1 |
| | | domain containing 1 | | | | |
| 6 | MAP7 | microtubule associated protein 7 | 135525 | 029363 | 197442 | 42, 13, 18 |
| 6 | MDN1 | midasin AAA ATPase 1 | 112159 | 083099 | 118412 | |
| 6 | MCM9 | minichromosome maintenance 9 homologous recombination | 111877 | 198523 | 111875 | 22, 37, 7 |
| | | repair factor | | | | |
| 6 | MCM3 | minichromosome maintenance complex component 3 | 112118 | 112116 | 170915 | 21, 39, 12 |
| 6 | MCUR1 | mitochondrial calcium uniporter regulator 1 | 050393 | 010017 | 180537 | 34, 29, 1 |
| 6 | MTCH1 | mitochondrial carrier 1 | 137409 | 164530 | 146192 | 34, 26, 40 |
| 6 | MCCD1 | mitochondrial coiled-coil domain 1 | 204511 | 204516 | 198563 | 17, 16, |
| 6 | MTFR2 | mitochondrial fission regulator 2 | 146410 | 171408 | 029363 | 3, 22, 13 |
| 6 | MPC1 | mitochondrial pyruvate carrier 1 | 060762 | 198818 | 071242 | 15, 45, 11 |
| 6 | MRPL14 | mitochondrial ribosomal protein L14 | 180992 | 181577 | 137216 | 25, 3, 34 |
| 6 | MRPL18 | mitochondrial ribosomal protein L18 | 112110 | 120438 | 146453 | 2, 30, 37 |
| 6 | MRPL2 | mitochondrial ribosomal protein L2 | 112651 | 137171 | 112655 | 43, 29, 6 |
| 6 | MRPS10 | mitochondrial ribosomal protein S10 | 048544 | 112599 | 124496 | 13, 3, 30 |
| 6 | MRPS18A | mitochondrial ribosomal protein S18A | 096080 | 172426 | 112715 | 21, 34, 35 |
| 6 | MRPS18B | mitochondrial ribosomal protein S18B | 204568 | 204569 | 137343 | 30, 1, 29 |
| 6 | MTRF1L | mitochondrial translational release factor 1 like | 112031 | 112029 | 091844 | 42, 17, 15 |
| 6 | MTO1 | mitochondrial tRNA translation optimization 1 | 135297 | 164430 | 156508 | 23, 36, 40 |
| 6 | MAPK13 | mitogen-activated protein kinase 13 | 156711 | 112062 | 096070 | 29, 18, 20 |
| 6 | MAPK14 | mitogen-activated protein kinase 14 | 112062 | 112053 | 156711 | 4, 1, 39 |
| 6 | MAP3K4 | mitogen-activated protein kinase kinase kinase 4 | 085511 | 122194 | 026652 | 30, 4, 29 |
| 6 | MAP3K5 | mitogen-activated protein kinase kinase kinase 5 | 197442 | 135525 | 112357 | 1, 22, 40 |
| 6 | MAP3K7 | mitogen-activated protein kinase kinase kinase 7 | 135341 | 112182 | 135333 | 11, 40, 45 |
| 6 | MMS22L | MMS22 like, DNA repair protein | 146263 | 186231 | 184486 | 9, 24, 10 |
| 6 | MOCS1 | molybdenum cofactor synthesis 1 | 124615 | 146122 | 156564 | 15, 26, 29 |
| 6 | MOXD1 | monooxygenase DBH like 1 | 079931 | 118523 | 079950 | 35, 10, 11 |
| 6 | MLN | motilin | 096395 | 161904 | 124493 | 37, |
| 6 | MRS2 | MRS2, magnesium transporter | 124532 | 146049 | 112293 | 29, 11, 22 |
| 6 | MSH5- | MSH5-SAPCD1 readthrough (NMD candidate) | 255152 | 204410 | 228727 | 39, 29, 38 |
| | SAPCD1 | | | | | |
| 6 | MUC21 | mucin 21, cell surface associated | 204544 | 168631 | 261272 | 13, 24, 30 |
| 6 | MUC22 | mucin 22 | 261272 | 204544 | 204542 | 14, |
| 6 | MLIP | muscular LMNA interacting protein | 146147 | 137269 | 137251 | 15, 26, 10 |
| 6 | MSH5 | mutS homolog 5 | 204410 | 213719 | 255152 | 39, 29, 38 |
| 6 | MYB | MYB proto-oncogene, transcription factor | 118513 | 112339 | 135541 | |
| 6 | MYCT1 | myc target 1 | 120279 | 131018 | 146469 | 43, 45, 1 |
| 6 | MOG | myelin oligodendrocyte glycoprotein | 204655 | 204657 | 204644 | 8, 17, 15 |
| 6 | MDFI | MyoD family inhibitor | 112559 | 137166 | 112561 | 12, 32, 4 |
| 6 | MYLK4 | myosin light chain kinase family member 4 | 145949 | 112699 | 124535 | 11, 18, 4 |
| 6 | MYLIP | myosin regulatory light chain interacting protein | 007944 | 047579 | 137198 | 29, 32, 26 |
| 6 | MYO6 | myosin VI | 196586 | 112701 | 112706 | 33, 11, 23 |
| 6 | MARCKS | myristoylated alanine rich protein kinase C substrate | 277443 | 251258 | 196591 | 11, 15, 20 |
| 6 | NQO2 | NAD(P)H quinone dehydrogenase 2 | 124588 | 124570 | 137275 | 34, 29, 9 |
| 6 | NDUFAF4 | NADH: ubiquinone oxidoreductase complex assembly factor 4 | 123545 | 112218 | 186231 | 29, 8, 25 |
| 6 | NOX3 | NADPH oxidase 3 | 074771 | 171217 | 049618 | 38, |
| 6 | NCR2 | natural cytotoxicity triggering receptor 2 | 096264 | 124731 | 137166 | 7, |
| 6 | NCR3 | natural cytotoxicity triggering receptor 3 | 204475 | 204482 | 204472 | 41, 5, 22 |
| 6 | NELFE | negative elongation factor complex member E | 204356 | 243649 | 204351 | 34, 43, 21 |
| 6 | NEDD9 | neural precursor cell expressed, developmentally | 111859 | 244476 | 205269 | 42, 45, 22 |
| | | down-regulated 9 | | | | |
| 6 | NEU1 | neuraminidase 1 | 204386 | 204387 | 204385 | 23, 2, 35 |
| 6 | NRSN1 | neurensin 1 | 152954 | 112273 | 146038 | 8, 31, 17 |
| 6 | NRN1 | neuritin 1 | 124785 | 145982 | 124491 | 34, 31, 6 |
| 6 | NMBR | neuromedin B receptor | 135577 | 164442 | 203733 | |
| 6 | NKAPL | NFKB activating protein like | 189134 | 187626 | 197062 | 31, 14, 2 |
| 6 | NFKBIE | NFKB inhibitor epsilon | 146232 | 157593 | 178233 | 41, 21, 9 |
| 6 | NFKBIL1 | NFKB inhibitor like 1 | 204498 | 213760 | 226979 | 18, 5, 26 |
| 6 | NHLRC1 | NHL repeat containing E3 ubiquitin protein ligase 1 | 187566 | 137177 | 137364 | 28, 38, 12 |
| 6 | NHSL1 | NHS like 1 | 135540 | 051620 | 279968 | 3, 16, 29 |
| 6 | NOTCH4 | notch 4 | 204301 | 213654 | 204296 | 26, 18, 32 |
| 6 | NCOA7 | nuclear receptor coactivator 7 | 111912 | 135547 | 111911 | 7, 42, 31 |
| 6 | NR2E1 | nuclear receptor subfamily 2 group E member 1 | 112333 | 081087 | 112335 | 8, 14, 32 |
| 6 | NFYA | nuclear transcription factor Y subunit alpha | 001167 | 124701 | 161911 | 11, 31, 1 |
| 6 | NOL7 | nucleolar protein 7 | 225921 | 124523 | 010017 | 29, 5, 26 |
| 6 | NUP153 | nucleoporin 153 | 124789 | 137414 | 137177 | 22, 32, 7 |
| 6 | NUP43 | nucleoporin 43 | 120253 | 278899 | 120265 | 29, 18, 34 |
| 6 | NUDT3 | nudix hydrolase 3 | 272325 | 186577 | 270800 | 29, 20, 26 |
| 6 | NRM | nurim (nuclear envelope membrane protein) | 137404 | 146112 | 137337 | 9, 27, 25 |
| 6 | NUS1 | NUS1 dehydrodolichyl diphosphate synthase subunit | 153989 | 047932 | 196376 | 26, 6, 4 |
| 6 | OARD1 | O-acyl-ADP-ribose deacylase 1 | 124596 | 124602 | 112212 | 34, 28, 10 |
| 6 | OR10C1 | olfactory receptor family 10 subfamily C member 1 (gene/pseudogene) | 206474 | 204694 | 204688 | |
| 6 | OR11A1 | olfactory receptor family 11 subfamily A member 1 | 204694 | 251608 | 206474 | |
| 6 | OR12D1 | olfactory receptor family 12 subfamily D member 1 (gene/pseudogene) | 251608 | 280236 | 204694 | |
| 6 | OR12D2 | olfactory receptor family 12 subfamily D member 2 (gene/pseudogene) | 280236 | 112462 | 251608 | |
| 6 | OR12D3 | olfactory receptor family 12 subfamily D member 3 | 112462 | 243729 | 280236 | |
| 6 | OR14J1 | olfactory receptor family 14 subfamily J member 1 | 204695 | 204700 | 243729 | |
| 6 | OR2A4 | olfactory receptor family 2 subfamily A member 4 | 180658 | 154269 | 236761 | 16, 30, 27 |
| 6 | OR2B2 | olfactory receptor family 2 subfamily B member 2 | 168131 | 274641 | 124657 | |
| 6 | OR2B3 | olfactory receptor family 2 subfamily B member 3 | 204703 | 204704 | 204702 | |
| 6 | OR2B6 | olfactory receptor family 2 subfamily B member 6 | 124657 | 168131 | 197279 | |
| 6 | OR2H1 | olfactory receptor family 2 subfamily H member 1 | 204688 | 206474 | 204687 | |
| 6 | OR2H2 | olfactory receptor family 2 subfamily H member 2 | 204657 | 204681 | 204655 | |
| 6 | OR2J1 | olfactory receptor family 2 subfamily J member 1 (gene/pseudogene) | 204702 | 204703 | 204701 | |
| 6 | OR2J2 | olfactory receptor family 2 subfamily J member 2 | 204700 | 204701 | 204695 | 38, |
| 6 | OR2J3 | olfactory receptor family 2 subfamily J member 3 | 204701 | 204702 | 204700 | |
| 6 | OR2W1 | olfactory receptor family 2 subfamily W member 1 | 204704 | 197935 | 204703 | 38, |
| 6 | OR5V1 | olfactory receptor family 5 subfamily V member 1 | 243729 | 204695 | 112462 | |
| 6 | OLIG3 | oligodendrocyte transcription factor 3 | 177468 | 027697 | 118503 | 27, 38, 12 |
| 6 | OOEP | oocyte expressed protein | 203907 | 203908 | 080007 | 32, 5, 35 |
| 6 | OGFRL1 | opioid growth factor receptor like 1 | 119900 | 112309 | 079841 | 18, 8, |
| 6 | OPRM1 | opioid receptor mu 1 | 112038 | 091844 | 074706 | 38, 8, |
| 6 | OPN5 | opsin 5 | 124818 | 153294 | 244694 | 38, |
| 6 | ORC3 | origin recognition complex subunit 3 | 135336 | 146282 | 135334 | 26, 13, 42 |
| 6 | OFCC1 | orofacial cleft 1 candidate 1 | 181355 | 124786 | 137203 | |
| 6 | OSTM1 | osteopetrosis associated transmembrane protein 1 | 081087 | 025796 | 112333 | |
| 6 | PAK1IP1 | PAK1 interacting protein 1 | 111845 | 137434 | 111843 | 6, 33, 30 |
| 6 | PPT2 | palmitoyl-protein thioesterase 2 | 221988 | 204314 | 258388 | 26, 39, 13 |
| 6 | PACRG | PARK2 coregulated | 112530 | 185345 | 112531 | 29, 38, 30 |
| 6 | PARK2 | parkin RBR E3 ubiquitin protein ligase | 185345 | 026652 | 112530 | 38, 24, 8 |
| 6 | PNLDC1 | PARN like, ribonuclease domain containing 1 | 146453 | 112110 | 130368 | 18, 24, 28 |
| 6 | PNPLA1 | patatin like phospholipase domain containing 1 | 180316 | 096070 | 189325 | 10, 17, |
| 6 | PTCHD4 | patched domain containing 4 | 244694 | 124818 | 146085 | 42, 32, 38 |
| 6 | PBX2 | PBX homeobox 2 | 204304 | 204305 | 213654 | 40, 29, 18 |
| 6 | PI16 | peptidase inhibitor 16 | 164530 | 198663 | 137409 | 43, 15, 10 |
| 6 | PM20D2 | peptidase M20 domain containing 2 | 146281 | 154548 | 146276 | 22, 31, 32 |
| 6 | PPIL1 | peptidylprolyl isomerase like 1 | 137168 | 124772 | 198663 | 4, 21, 13 |
| 6 | PPIL4 | peptidylprolyl isomerase like 4 | 131013 | 178199 | 055211 | 30, 1, 43 |
| 6 | PPIL6 | peptidylprolyl isomerase like 6 | 185250 | 135535 | 135587 | 23, 38, 3 |
| 6 | PRPH2 | peripherin 2 | 112619 | 024048 | 124659 | 25, 43, 14 |
| 6 | PEX3 | peroxisomal biogenesis factor 3 | 034693 | 189007 | 001036 | |
| 6 | PEX6 | peroxisomal biogenesis factor 6 | 124587 | 124713 | 112640 | 1, 21, 9 |
| 6 | PEX7 | peroxisomal biogenesis factor 7 | 112357 | 197442 | 182747 | 43, 10, 16 |
| 6 | PXT1 | peroxisomal, testis specific 1 | 179165 | 010030 | 112078 | 38, 17, |
| 6 | PPARD | peroxisome proliferator activated receptor delta | 112033 | 023892 | 112039 | 43, 32, 27 |
| 6 | PERP | PERP, TP53 apoptosis effector | 112378 | 118503 | 112379 | 13, 42, 4 |
| 6 | PHF1 | PHD finger protein 1 | 112511 | 237649 | 112514 | 26, 38, 33 |
| 6 | PHF10 | PHD finger protein 10 | 130024 | 185127 | 184786 | 1, 22, 29 |
| 6 | PHF3 | PHD finger protein 3 | 118482 | 112245 | 188107 | 31, 11, 30 |
| 6 | FARS2 | phenylalanyl-tRNA synthetase 2, mitochondrial | 145982 | 214113 | 124785 | 43, 10, 6 |
| 6 | PHACTR1 | phosphatase and actin regulator 1 | 112137 | 078401 | 145979 | 22, 35, 7 |
| 6 | PHACTR2 | phosphatase and actin regulator 2 | 112419 | 001036 | 257065 | 32, 26, 1 |
| 6 | PDE10A | phosphodiesterase 10A | 112541 | 112539 | 164458 | |
| 6 | PDE7B | phosphodiesterase 7B | 171408 | 135541 | 146410 | 11, 31, 25 |
| 6 | PGM3 | phosphoglucomutase 3 | 013375 | 281446 | 013392 | 45, 6, 38 |
| 6 | PGK2 | phosphoglycerate kinase 2 | 170950 | 096006 | 124812 | 38, 25, 8 |
| 6 | PLN | phospholamban | 198523 | 111860 | 111877 | 15, 25, 45 |
| 6 | PLA2G7 | phospholipase A2 group VII | 146070 | 180113 | 230062 | 30, 36, 32 |
| 6 | PGBD1 | piggyBac transposable element derived 1 | 137338 | 276302 | 235109 | 13, 43, 9 |
| 6 | PIM1 | Pim-1 proto-oncogene, serine/threonine kinase | 137193 | 146192 | 172738 | 24, 35, 22 |
| 6 | PLAGL1 | PLAG1 like zinc finger 1 | 118495 | 118491 | 169976 | 2, 32, 36 |
| 6 | PLG | plasminogen | 122194 | 198670 | 085511 | 19, 17, 36 |
| 6 | PLEKHG1 | pleckstrin homology and RhoGEF domain containing G1 | 120278 | 009765 | 120254 | 36, 22, 1 |
| 6 | PHIP | pleckstrin homology domain interacting protein | 146247 | 146243 | 118418 | |
| 6 | PNISR | PNN interacting serine and arginine rich protein | 132424 | 132423 | 123552 | |
| 6 | PKHD1 | polycystic kidney and hepatic disease 1 (autosomal recessive) | 170927 | 008196 | 112115 | 17, 2, 38 |
| 6 | POM121L2 | POM121 transmembrane nucleoporin like 2 | 158553 | 112812 | 124613 | |
| 6 | POPDC3 | popeye domain containing 3 | 132429 | 112276 | 085377 | 35, 28, 25 |
| 6 | KCTD20 | potassium channel tetramerization domain containing 20 | 112078 | 179165 | 112079 | 22, 9, 42 |
| 6 | KCNK16 | potassium two pore domain channel subfamily K member 16 | 095981 | 124780 | 164627 | 26, 28, |
| 6 | KCNK17 | potassium two pore domain channel subfamily K member 17 | 124780 | 164626 | 095981 | 11, 32, 39 |
| 6 | KCNK5 | potassium two pore domain channel subfamily K member 5 | 164626 | 112167 | 124780 | 7, 17, 1 |
| 6 | KCNQ5 | potassium voltage-gated channel subfamily Q member 5 | 185760 | 079841 | 243501 | |
| 6 | POU3F2 | POU class 3 homeobox 2 | 184486 | 146263 | 112234 | 45, 35, 12 |
| 6 | POU5F1 | POU class 5 homeobox 1 | 204531 | 137310 | 206344 | 12, 27, 37 |
| 6 | PPT2-EGFL8 | PPT2-EGFL8 readthrough | 258388 | 221988 | 241404 | 26, 35, 3 |
| 6 | PRDM1 | PR/SET domain 1 | 057657 | 057663 | 283623 | 24, 18, 22 |
| 6 | PRDM13 | PR/SET domain 13 | 112238 | 112237 | 152034 | |
| 6 | PTCRA | pre T-cell antigen receptor alpha | 171611 | 221821 | 137161 | 36, 39, 17 |
| 6 | PFDN6 | prefoldin subunit 6 | 204220 | 227057 | 237441 | 29, 24, 3 |
| 6 | PRPF4B | pre-mRNA processing factor 4B | 112739 | 145945 | 145975 | 42, 39, 7 |
| 6 | PRICKLE4 | prickle planar cell polarity protein 4 | 124593 | 137218 | 278224 | 36, 42, 15 |
| 6 | PRICKLE4 | prickle planar cell polarity protein 4 | 278224 | 124593 | 214736 | 36, 42, 15 |
| 6 | PRIM2 | primase (DNA) subunit 2 | 146143 | 112210 | 279892 | 13, 45, 10 |
| 6 | PGC | progastricsin | 096088 | 112561 | 137218 | 37, 3, 13 |
| 6 | PAQR8 | progestin and adipoQ receptor family member 8 | 170915 | 112118 | 096093 | 26, 16, 8 |
| 6 | PDCD2 | programmed cell death 2 | 071994 | 112592 | 172426 | 22, 27, 4 |
| 6 | PRL | prolactin | 172179 | 124766 | 112273 | |
| 6 | PRR18 | proline rich 18 | 176381 | 164458 | 198818 | 26, 30, 8 |
| 6 | PRR3 | proline rich 3 | 204576 | 204590 | 204574 | 13, 9, 25 |
| 6 | PRRC2A | proline rich coiled-coil 2A | 204469 | 204472 | 204463 | 21, 22, 26 |
| 6 | PNRC1 | proline rich nuclear receptor coactivator 1 | 146278 | 111880 | 154548 | 11, 5, 37 |
| 6 | PRRT1 | proline rich transmembrane protein 1 | 204314 | 204315 | 221988 | |
| 6 | PREP | prolyl endopeptidase | 085377 | 132429 | 057663 | 45, 31, 3 |
| 6 | PB0V1 | prostate and breast cancer overexpressed 1 | 254440 | 112379 | 262543 | |
| 6 | PRSS16 | protease, serine 16 | 112812 | 274997 | 158553 | 39, 13, 29 |
| 6 | PRSS35 | protease, serine 35 | 146250 | 065833 | 065609 | 43, 11, 6 |
| 6 | PSMG4 | proteasome assembly chaperone 4 | 180822 | 137285 | 137266 | |
| 6 | PSMB1 | proteasome subunit beta 1 | 008018 | 112584 | 112592 | 34, 30, 43 |
| 6 | PSMB8 | proteasome subunit beta 8 | 204264 | 204267 | 240065 | 34, 22, 5 |
| 6 | PSMB9 | proteasome subunit beta 9 | 240065 | 204264 | 168394 | 24, 18, 30 |
| 6 | PACSIN1 | protein kinase C and casein kinase substrate in neurons 1 | 124507 | 124614 | 124664 | 14, 26, 41 |
| 6 | PPP1R11 | protein phosphatase 1 regulatory inhibitor subunit 11 | 204619 | 066379 | 204618 | 35, 7, 9 |
| 6 | PPP1R14C | protein phosphatase 1 regulatory inhibitor subunit 14C | 198729 | 131019 | 009765 | 40, 29, 24 |
| 6 | PPP1R10 | protein phosphatase 1 regulatory subunit 10 | 204569 | 204574 | 204568 | 26, 7, 21 |
| 6 | PPP1R18 | protein phosphatase 1 regulatory subunit 18 | 146112 | 204560 | 137404 | 22, 41, 43 |
| 6 | PPP1R3G | protein phosphatase 1 regulatory subunit 3G | 219607 | 124787 | 214113 | 36, 19, |
| 6 | PPP2R5D | protein phosphatase 2 regulatory subunit B′delta | 112640 | 124587 | 124733 | 21, 39, 35 |
| 6 | PTK7 | protein tyrosine kinase 7 (inactive) | 112655 | 112651 | 112658 | 39, 12, 24 |
| 6 | PTP4A1 | protein tyrosine phosphatase type IVA, member 1 | 112245 | 146166 | 118482 | 9, 24, 19 |
| 6 | PTPRK | protein tyrosine phosphatase, receptor type K | 152894 | 172673 | 196569 | 30, 4, 16 |
| 6 | PCMT1 | protein-L-isoaspartate (D-aspartate) O-methyltransferase | 120265 | 120253 | 120256 | 1, 8, 13 |
| 6 | PSORS1C1 | psoriasis susceptibility 1 candidate 1 | 204540 | 204542 | 204539 | 18, 35, 32 |
| 6 | PSORS1C2 | psoriasis susceptibility 1 candidate 2 | 204538 | 204539 | 204536 | 35, 15, |
| 6 | PXDC1 | PX domain containing 1 | 168994 | 137266 | 145945 | 26, 32, 1 |
| 6 | QKI | QKI, KH domain containing RNA binding | 112531 | 112530 | 112539 | 26, 11, 8 |
| 6 | RAB23 | RAB23, member RAS oncogene family | 112210 | 112208 | 146143 | 45, 6, 44 |
| 6 | RAB32 | RAB32, member RAS oncogene family | 118508 | 152822 | 118492 | 43, 15, 35 |
| 6 | RAB44 | RAB44, member RAS oncogene family | 255587 | 124762 | 124772 | 5, 7, |
| 6 | RSPH3 | radial spoke 3 homolog | 130363 | 092820 | 164691 | 11, 38, 31 |
| 6 | RSPH4A | radial spoke head 4 homolog A | 111834 | 111832 | 153975 | 42, 30, 31 |
| 6 | RSPH9 | radial spoke head 9 homolog | 172426 | 124688 | 096080 | 43, 29, 14 |
| 6 | RGL2 | ral guanine nucleotide dissociation stimulator like 2 | 237441 | 204220 | 231925 | 40, 26, 36 |
| 6 | REPS1 | RALBP1 associated Eps domain containing 1 | 135597 | 203734 | 146386 | 31, 7, 23 |
| 6 | RANBP9 | RAN binding protein 9 | 010017 | 225921 | 050393 | 24, 18, 15 |
| 6 | RRAGD | Ras related GTP binding D | 025039 | 198833 | 135299 | 29, 26, 11 |
| 6 | RREB1 | ras responsive element binding protein 1 | 124782 | 112799 | 124783 | 35, 18, 5 |
| 6 | RIPK1 | receptor interacting serine/threonine kinase 1 | 137275 | 124588 | 137274 | 4, 13, 3 |
| 6 | RIMS1 | regulating synaptic membrane exocytosis 1 | 079841 | 119900 | 185760 | 8, 11, 40 |
| 6 | RCAN2 | regulator of calcineurin 2 | 172348 | 112796 | 146233 | 8, 29, 26 |
| 6 | RGS17 | regulator of G-protein signaling 17 | 091844 | 112031 | 112038 | 10, 23, 45 |
| 6 | RFX6 | regulatory factor X6 | 185002 | 173612 | 170162 | 28, 2, 10 |
| 6 | RMND1 | required for meiotic nuclear division 1 homolog | 155906 | 181472 | 146476 | 41, 13, 2 |
| 6 | RFPL4B | ret finger protein like 4B | 251258 | 281613 | 277443 | 38, |
| 6 | RTN4IP1 | reticulon 4 interacting protein 1 | 130347 | 112297 | 130348 | 41, 31, 10 |
| 6 | RAET1E | retinoic acid early transcript 1E | 164520 | 120256 | 203722 | 9, 36, 35 |
| 6 | RAET1G | retinoic acid early transcript 1G | 203722 | 164520 | 131015 | 30, 18, 2 |
| 6 | RAET1L | retinoic acid early transcript 1L | 155918 | 111981 | 131019 | 24, 25, |
| 6 | RXRB | retinoid X receptor beta | 204231 | 204248 | 112473 | 29, 9, 15 |
| 6 | REV3L | REV3 like, DNA directed polymerase zeta catalytic subunit | 009413 | 173214 | 056972 | 1, 43, 25 |
| 6 | RHAG | Rh-associated glycoprotein | 112077 | 197261 | 124490 | 7, 34, 5 |
| 6 | ARHGAP18 | Rho GTPase activating protein 18 | 146376 | 196569 | 203756 | 42, 45, 30 |
| 6 | RPP21 | ribonuclease P/MRP subunit p21 | 241370 | 248167 | 204592 | 43, 45, 14 |
| 6 | RPP40 | ribonuclease P/MRP subunit p40 | 124787 | 153046 | 219607 | 6, 18, 4 |
| 6 | RNASET2 | ribonuclease T2 | 026297 | 249141 | 213066 | 36, 18, 16 |
| 6 | RPL10A | ribosomal protein L10a | 198755 | 112039 | 007866 | |
| 6 | RPL7L1 | ribosomal protein L7 like 1 | 146223 | 112624 | 221821 | 30, 19, 24 |
| 6 | RPS10 | ribosomal protein S10 | 124614 | 270800 | 124507 | |
| 6 | RPS12 | ribosomal protein S12 | 112306 | 146409 | 112319 | 43, 12, 41 |
| 6 | RPS18 | ribosomal protein S18 | 231500 | 223501 | 235863 | |
| 6 | RPS6KA2 | ribosomal protein S6 kinase A2 | 071242 | 060762 | 249141 | 40, 8, 23 |
| 6 | RRP36 | ribosomal RNA processing 36 | 124541 | 124702 | 044090 | 34, 44, 37 |
| 6 | RPF2 | ribosome production factor 2 homolog | 197498 | 155115 | 112394 | 34, 4, 37 |
| 6 | RING1 | ring finger protein 1 | 204227 | 204228 | 223501 | 9, 1, 14 |
| 6 | RNF144B | ring finger protein 144B | 137393 | 124795 | 172201 | 40, 11, 42 |
| 6 | RNF146 | ring finger protein 146 | 118518 | 146374 | 093144 | 29, 11, 9 |
| 6 | RNF182 | ring finger protein 182 | 180537 | 050393 | 112149 | 8, 9, 38 |
| 6 | RNF217 | ring finger protein 217 | 146373 | 188580 | 111907 | 8, |
| 6 | RNF39 | ring finger protein 39 | 204618 | 204619 | 204616 | 18, 15, 16 |
| 6 | RNF5 | ring finger protein 5 | 204308 | 204310 | 204305 | 27, 3, 13 |
| 6 | RNF8 | ring finger protein 8 | 112130 | 065491 | 137200 | 27, 5, 38 |
| 6 | RIOK1 | RIO kinase 1 | 124784 | 164304 | 096696 | 10, 3, 9 |
| 6 | RIPPLY2 | ripply transcriptional repressor 2 | 203877 | 065609 | 065615 | 26, 28, 37 |
| 6 | RBM24 | RNA binding motif protein 24 | 112183 | 230873 | 112186 | 15, 25, 18 |
| 6 | RNGTT | RNA guanylyltransferase and 5′-phosphatase | 111880 | 118432 | 146278 | 31, 22, 42 |
| 6 | POLR1C | RNA polymerase I subunit C | 171453 | 204052 | 137207 | 1, 43, 5 |
| 6 | ROS1 | ROS proto-oncogene 1, receptor tyrosine kinase | 047936 | 170162 | 282218 | 45, 20, 10 |
| 6 | RPS10- | RPS10-NUDT3 readthrough | 270800 | 272325 | 124614 | |
| | NUDT3 | | | | | |
| 6 | RSPO3 | R-spondin 3 | 146374 | 203760 | 118518 | 1, 15, 44 |
| 6 | RUNX2 | runt related transcription factor 2 | 124813 | 283553 | 112782 | 11, 18, 27 |
| 6 | RWDD1 | RWD domain containing 1 | 111832 | 164451 | 111834 | 22, 15, 30 |
| 6 | RWDD2A | RWD domain containing 2A | 013392 | 013375 | 065833 | 31, 11, 33 |
| 6 | SASH1 | SAM and SH3 domain containing 1 | 111961 | 203727 | 111962 | 35, 1, 11 |
| 6 | SPDEF | SAM pointed domain containing ETS transcription factor | 124664 | 124507 | 196821 | 23, 27, 33 |
| 6 | SAYSD1 | SAYSVFN motif domain containing 1 | 112167 | 112164 | 164626 | 38, 11, 15 |
| 6 | SEC63 | SEC63 homolog, protein translocation regulator | 025796 | 146285 | 081087 | 18, 11, 1 |
| 6 | SCGN | secretagogin, EF-hand calcium binding protein | 079689 | 079691 | 164508 | 28, 20, 31 |
| 6 | SERAC1 | serine active site containing 1 | 122335 | 078269 | 272047 | 38, 42, 10 |
| 6 | SRSF12 | serine and arginine rich splicing factor 12 | 154548 | 146278 | 146281 | 38, 8, 27 |
| 6 | SRSF3 | serine and arginine rich splicing factor 3 | 112081 | 112079 | 124762 | 4, 7, 13 |
| 6 | SERINC1 | serine incorporator 1 | 111897 | 025156 | 135549 | 42, 39, 8 |
| 6 | STK19 | serine/threonine kinase 19 | 204344 | 204348 | 244731 | 1, 36, 2 |
| 6 | STK38 | serine/threonine kinase 38 | 112079 | 112078 | 112081 | 24, 5, 42 |
| 6 | SERPINB1 | serpin family B member 1 | 021355 | 124535 | 170542 | 7, 37, 5 |
| 6 | SERPINB6 | serpin family B member 6 | 124570 | 170542 | 124588 | 24, 10, 3 |
| 6 | SERPINB9 | serpin family B member 9 | 170542 | 021355 | 124570 | 34, 22, 21 |
| 6 | SRF | serum response factor | 112658 | 112655 | 112659 | 21, 1, 29 |
| 6 | SGK1 | serum/glucocorticoid regulated kinase 1 | 118515 | 146411 | 118514 | 45, 29, 2 |
| 6 | SESN1 | sestrin 1 | 080546 | 118690 | 183137 | 31, 38, 33 |
| 6 | SCML4 | sex comb on midleg-like 4 (Drosophila) | 146285 | 112320 | 025796 | 35, 39, 40 |
| 6 | SFT2D1 | SFT2 domain containing 1 | 198818 | 176381 | 060762 | 41, 11, 29 |
| 6 | SH3BGRL2 | SH3 domain binding glutamate rich protein like 2 | 198478 | 135338 | 118402 | 42, 11, 16 |
| 6 | SCUBE3 | signal peptide, CUB domain and EGF like domain containing 3 | 146197 | 124678 | 065029 | 40, 20, 27 |
| 6 | SSR1 | signal sequence receptor subunit 1 | 124783 | 124782 | 164304 | 30, 19, 24 |
| 6 | SOBP | sine oculis binding protein homolog | 112320 | 164494 | 146285 | 11, 34, 20 |
| 6 | SIM1 | single-minded family bHLH transcription factor 1 | 112246 | 152034 | 112249 | |
| 6 | SIRT5 | sirtuin 5 | 124523 | 145990 | 225921 | |
| 6 | SKIV2L | Ski2 like RNA helicase | 204351 | 204356 | 204348 | 3, 41, 9 |
| 6 | SMAP1 | small ArfGAP 1 | 112305 | 154079 | 112309 | 11, 22, 43 |
| 6 | SMIM13 | small integral membrane protein 13 | 224531 | 197977 | 244476 | 10, 45, 29 |
| 6 | SMIM8 | small integral membrane protein 8 | 111850 | 164411 | 203872 | |
| 6 | SMLR1 | small leucine rich protein 1 | 256162 | 164484 | 079819 | 17, 36, 19 |
| 6 | SNRPC | small nuclear ribonucleoprotein polypeptide C | 124562 | 196821 | 065060 | 43, 19, 29 |
| 6 | SNRNP48 | small nuclear ribonucleoprotein U11/U12 subunit 48 | 168566 | 096696 | 153162 | 29, 15, 26 |
| 6 | SLIMO4 | small ubiquitin-like modifier 4 | 177688 | 055208 | 178199 | 25, |
| 6 | SHPRH | SNF2 histone linker PHD RING helicase | 146414 | 118496 | 152822 | 31, 43, 39 |
| 6 | NKAIN2 | Sodium/potassium transporting ATPase interacting 2 | 188580 | 186439 | 146373 | 8, 10, 26 |
| 6 | SOGA3 | SOGA family member 3 | 214338 | 189367 | 184530 | 31, 8, 38 |
| 6 | SLC16A10 | solute carrier family 16 member 10 | 112394 | 197498 | 173214 | 29, 39, 10 |
| 6 | SLC17A1 | solute carrier family 17 member 1 | 124568 | 146039 | 124564 | 17, 19, 25 |
| 6 | SLC17A2 | solute carrier family 17 member 2 | 112337 | 124564 | 112343 | 19, 5, 17 |
| 6 | SLC17A3 | solute carrier family 17 member 3 | 124564 | 124568 | 112337 | 17, 8, 19 |
| 6 | SLC17A4 | solute carrier family 17 member 4 | 146039 | 146047 | 124568 | 16, 4, 19 |
| 6 | SLC17A5 | solute carrier family 17 member 5 | 119899 | 156508 | 156535 | 29, 42, 26 |
| 6 | SLC18B1 | solute carrier family 18 member B1 | 146409 | 112303 | 112306 | 34, 19, 40 |
| 6 | SLC2A12 | solute carrier family 2 member 12 | 146411 | 028839 | 118515 | 42, 10, 29 |
| 6 | SLC22A1 | solute carrier family 22 member 1 | 175003 | 197081 | 112499 | 19, 23, 10 |
| 6 | SLC22A16 | solute carrier family 22 member 16 | 004809 | 203797 | 155111 | 10, 38, 36 |
| 6 | SLC22A2 | solute carrier family 22 member 2 | 112499 | 175003 | 146477 | 17, 10, 32 |
| 6 | SLC22A23 | solute carrier family 22 member 23 | 137266 | 180822 | 168994 | 29, 42, 16 |
| 6 | SLC22A3 | solute carrier family 22 member 3 | 146477 | 112499 | 198670 | 11, 33, 19 |
| 6 | SLC22A7 | solute carrier family 22 member 7 | 137204 | 146216 | 146215 | 19, 17, 15 |
| 6 | SLC25A27 | solute carrier family 25 member 27 | 153291 | 146233 | 180113 | 31, 29, 1 |
| 6 | SLC26A8 | solute carrier family 26 member 8 | 112053 | 096063 | 112062 | 38, 1, 40 |
| 6 | SLC29A1 | solute carrier family 29 member 1 (Augustine blood group) | 112759 | 262179 | 096384 | 43, 2, 39 |
| 6 | SLC35A1 | solute carrier family 35 member A1 | 164414 | 213204 | 146282 | 11, 4, 22 |
| 6 | SLC35B2 | solute carrier family 35 member B2 | 157593 | 096384 | 146232 | 35, 4, 13 |
| 6 | SLC35B3 | solute carrier family 35 member B3 | 124786 | 124802 | 181355 | 31, 22, 13 |
| 6 | SLC35D3 | solute carrier family 35 member D3 | 182747 | 112357 | 016402 | 26, 20, 28 |
| 6 | SLC35F1 | solute carrier family 35 member F1 | 196376 | 153989 | 111860 | 11, 2, 8 |
| 6 | SLC39A7 | solute carrier family 39 member 7 | 112473 | 204231 | 204228 | 13, 45, 26 |
| 6 | SLC44A4 | solute carrier family 44 member 4 | 204385 | 204386 | 204371 | 42, 28, 33 |
| 6 | SNX14 | sorting nexin 14 | 135317 | 135318 | 271793 | 40, 38, 27 |
| 6 | SNX3 | sorting nexin 3 | 112335 | 112333 | 135537 | 34, 31, 29 |
| 6 | SNX9 | sorting nexin 9 | 130340 | 175048 | 078269 | 1, 45, 10 |
| 6 | SMOC2 | SPARC related modular calcium binding 2 | 112562 | 164488 | 186340 | 11, 1, 10 |
| 6 | SYNE1 | spectrin repeat containing nuclear envelope protein 1 | 131018 | 091831 | 120279 | |
| 6 | SPACA1 | sperm acrosome associated 1 | 118434 | 135334 | 118432 | 38, 8, |
| 6 | SPATS1 | spermatogenesis associated serine rich 1 | 249481 | 124608 | 096401 | 38, 42, 35 |
| 6 | SMPD2 | sphingomyelin phosphodiesterase 2 | 135587 | 185250 | 135596 | 37, 22, 14 |
| 6 | SMPDL3A | sphingomyelin phosphodiesterase acid like 3A | 172594 | 164434 | 146352 | 11, 32, 37 |
| 6 | SF3B5 | splicing factor 3b subunit 5 | 169976 | 118495 | 135604 | 29, 2, 41 |
| 6 | SUPT3H | SPT3 homolog, SAGA and STAGA complex component | 196284 | 096401 | 283553 | 2, 1, 10 |
| 6 | SCAF8 | SR-related CTD associated factor 8 | 213079 | 153721 | 146426 | 22, 1, 38 |
| 6 | SRPK1 | SRSF protein kinase 1 | 096063 | 197753 | 112053 | 42, 38, 24 |
| 6 | SOX4 | SRY-box4 | 124766 | 145996 | 172179 | 7, 19, 15 |
| 6 | STMND1 | stathmin domain containing 1 | 230873 | 124788 | 112183 | 20, |
| 6 | SAMD3 | sterile alpha motif domain containing 3 | 164483 | 198945 | 164484 | 10, 38, 36 |
| 6 | SAMD5 | sterile alpha motif domain containing 5 | 203727 | 164506 | 111961 | 11, 24, 18 |
| 6 | SDHAF4 | succinate dehydrogenase complex assembly factor 4 | 154079 | 082269 | 112305 | |
| 6 | SENP6 | SUMO1/sentrin specific peptidase 6 | 112701 | 118407 | 196586 | 31, 11, 21 |
| 6 | SOD2 | superoxide dismutase 2 | 112096 | 164694 | 146457 | 7, 21, 5 |
| 6 | SAPCD1 | suppressor APC domain containing 1 | 228727 | 255152 | 204396 | 3, 27, 33 |
| 6 | SFTA2 | surfactant associated 2 | 196260 | 137411 | 168631 | 20, 16, |
| 6 | SYNGAP1 | synaptic Ras GTPase activating protein 1 | 197283 | 112514 | 213588 | 2, 28, 14 |
| 6 | SYNJ2 | synaptojanin 2 | 078269 | 130340 | 122335 | 38, 9, 45 |
| 6 | SYCP2L | synaptonemal complex protein 2 like | 153157 | 124827 | 197977 | 29, 4, 3 |
| 6 | SNAP91 | synaptosome associated protein 91 | 065609 | 146250 | 203877 | 8, 26, 31 |
| 6 | SYNCRIP | synaptotagmin binding cytoplasmic RNA interacting protein | 135316 | 271793 | 168830 | 4, 6, 1 |
| 6 | SYTL3 | synaptotagmin like 3 | 164674 | 146425 | 092820 | 39, 42, 30 |
| 6 | STX11 | syntaxin 11 | 135604 | 169976 | 152818 | 22, 7, 1 |
| 6 | STX7 | syntaxin 7 | 079950 | 079931 | 237110 | 22, 42, 8 |
| 6 | STXBP5 | syntaxin binding protein 5 | 164506 | 118492 | 203727 | 8, |
| 6 | T | T brachyury transcription factor | 164458 | 112541 | 176381 | 20, 23, 10 |
| 6 | TAPBP | TAP binding protein | 231925 | 237441 | 236104 | 9, 26, 6 |
| 6 | TBP | TATA-box binding protein | 112592 | 008018 | 071994 | 31, 37, 18 |
| 6 | TAF11 | TATA-box binding protein associated factor 11 | 064995 | 065060 | 064999 | 39, 34, 21 |
| 6 | TAF8 | TATA-box binding protein associated factor 8 | 137413 | 112576 | 188112 | 3, 34, 29 |
| 6 | TBPL1 | TATA-box binding protein like 1 | 028839 | 118526 | 146411 | 34, 7, 38 |
| 6 | TTBK1 | tau tubulin kinase 1 | 146216 | 112667 | 137204 | 8, 27, 44 |
| 6 | TXLNB | taxilin beta | 164440 | 112406 | 164442 | 25, 15, 31 |
| 6 | TBC1D22B | TBC1 domain family member 22B | 065491 | 172738 | 112130 | 24, 42, 39 |
| 6 | TBC1D32 | TBC1 domain family member 32 | 146350 | 111885 | 152661 | 18, 42, 29 |
| 6 | TBC1D7 | TBC1 domain family member 7 | 145979 | 112137 | 145990 | 35, 34, 22 |
| 6 | TBX18 | T-box 18 | 112837 | 135315 | 135318 | |
| 6 | TAGAP | T-cell activation RhoGTPase activating protein | 164691 | 130363 | 164694 | 5, 39, 10 |
| 6 | TIAM2 | T-cell lymphoma invasion and metastasis 2 | 146426 | 213079 | 029639 | |
| 6 | TCP1 | t-complex 1 | 120438 | 120437 | 112110 | 39, 43, 13 |
| 6 | TCP10 | t-complex 10 | 203690 | 120440 | 130396 | 38, 27, 28 |
| 6 | TCP10L2 | t-complex 10-like 2 | 166984 | 112486 | 120436 | |
| 6 | TCP11 | t-complex 11 | 124678 | 064999 | 146197 | 38, 8, 10 |
| 6 | TCTE1 | t-complex-associated-testis-expressed 1 | 146221 | 272442 | 124608 | 38, 42, 27 |
| 6 | TCTE3 | t-complex-associated-testis-expressed 3 | 184786 | 130024 | 130023 | 40, 23, 2 |
| 6 | TEAD3 | TEA domain transcription factor 3 | 007866 | 198755 | 112041 | 32, 34, 12 |
| 6 | TNXB | tenascin XB | 168477 | 231852 | 213676 | 1, 2, 26 |
| 6 | TAB2 | TGF-beta activated kinase 1/MAP3K7 binding protein 2 | 055208 | 111962 | 177688 | 29, 43, 11 |
| 6 | TPMT | thiopurine S-methyltransferase | 137364 | 187566 | 165097 | 7, 40, 13 |
| 6 | TXNDC5 | thioredoxin domain containing 5 | 239264 | 153162 | 259040 | 43, 21, 35 |
| 6 | TSTD3 | thiosulfate sulfurtransferase (rhodanese)-like domain | 228439 | 123552 | 112237 | 29, 25, 23 |
| | | containing 3 | | | | |
| 6 | THBS2 | thrombospondin 2 | 186340 | 112562 | 184465 | 11, 6, 1 |
| 6 | THEMIS | thymocyte selection associated | 172673 | 184530 | 152894 | 39, 42, 36 |
| 6 | TJAP1 | tight junction associated protein 1 | 137221 | 171462 | 204052 | 24, 39, 43 |
| 6 | TNFAIP3 | TNF alpha induced protein 3 | 118503 | 177468 | 112378 | 42, 22, 39 |
| 6 | TNFRSF21 | TNF receptor superfamily member 21 | 146072 | 153292 | 198087 | 4, 42, 24 |
| 6 | TAAR1 | trace amine associated receptor 1 | 146399 | 146378 | 112299 | 37, |
| 6 | TAAR2 | trace amine associated receptor 2 | 146378 | 135569 | 146399 | |
| 6 | TAAR5 | trace amine associated receptor 5 | 135569 | 146383 | 146378 | |
| 6 | TAAR6 | trace amine associated receptor 6 | 146383 | 146385 | 135569 | |
| 6 | TAAR8 | trace amine associated receptor 8 | 146385 | 237110 | 146383 | |
| 6 | TAAR9 | trace amine associated receptor 9 (gene/pseudogene) | 237110 | 079950 | 146385 | |
| 6 | TRAF3IP2 | TRAF3 interacting protein 2 | 056972 | 009413 | 010810 | |
| 6 | TRAPPC3L | trafficking protein particle complex 3 like | 173626 | 188820 | 178033 | 7, 19, 32 |
| 6 | TCF19 | transcription factor 19 | 137310 | 204536 | 204531 | 39, 13, 3 |
| 6 | TCF21 | transcription factor 21 | 118526 | 112319 | 028839 | 32, 27, 36 |
| 6 | TFAP2A | transcription factor AP-2 alpha | 137203 | 181355 | 111846 | 32, 9, 23 |
| 6 | TFAP2B | transcription factor AP-2 beta | 008196 | 008197 | 170927 | 14, 12, 23 |
| 6 | TFAP2D | transcription factor AP-2 delta | 008197 | 226733 | 008196 | 14, 24, 8 |
| 6 | TFB1M | transcription factor B1, mitochondrial | 029639 | 146426 | 171217 | 4, 29, 6 |
| 6 | TFEB | transcription factor EB | 112561 | 112559 | 096088 | 7, 21, 22 |
| 6 | TRERF1 | transcriptional regulating factor 1 | 124496 | 048544 | 024048 | 1, 39, 3 |
| 6 | TOMM6 | translocase of outer mitochondrial membrane 6 | 214736 | 278224 | 164663 | 3, 2, 7 |
| 6 | TRAM2 | translocation associated membrane protein 2 | 065308 | 096093 | 096092 | 45, 11, 19 |
| 6 | TSPO2 | translocator protein 2 | 112212 | 124596 | 124701 | 5, |
| 6 | TMEM14A | transmembrane protein 14A | 096092 | 065308 | 244067 | 4, 2, 7 |
| 6 | TMEM14B | transmembrane protein 14B | 137210 | 111843 | 272162 | 45, 4, 2 |
| 6 | TMEM14C | transmembrane protein 14C | 111843 | 111845 | 137210 | 35, 31, 28 |
| 6 | TMEM151B | transmembrane protein 151B | 178233 | 146232 | 272442 | 26, 8, 20 |
| 6 | TMEM170B | transmembrane protein 170B | 205269 | 111859 | 111863 | 1, 29, 25 |
| 6 | TMEM181 | transmembrane protein 181 | 146433 | 130338 | 146425 | 29, 26, 12 |
| 6 | TMEM200A | transmembrane protein 200A | 164484 | 164483 | 256162 | 11, 31, 2 |
| 6 | TMEM217 | transmembrane protein 217 | 172738 | 137193 | 065491 | 4, 38, 9 |
| 6 | TMEM242 | transmembrane protein 242 | 215712 | 049618 | 175048 | 29, 1, 43 |
| 6 | TMEM244 | transmembrane protein 244 | 203756 | 146376 | 198945 | 14, |
| 6 | TMEM30A | transmembrane protein 30A | 112697 | 112695 | 118407 | 42, 13, 29 |
| 6 | TMEM63B | transmembrane protein 63B | 137216 | 180992 | 137225 | 18, 14, 35 |
| 6 | TAP1 | transporter 1, ATP binding cassette subfamily B member | 168394 | 240065 | 242574 | 36, 39, 13 |
| 6 | TAP2 | transporter 2, ATP binding cassette subfamily B member | 204267 | 250264 | 204264 | 4, 24, 37 |
| 6 | TRDN | triadin | 186439 | 146352 | 188580 | 25, 15, 26 |
| 6 | TREM1 | triggering receptor expressed on myeloid cells 1 | 124731 | 188056 | 096264 | 5, 32, 36 |
| 6 | TREM2 | triggering receptor expressed on myeloid cells 2 | 095970 | 161911 | 112195 | 1, 9, 28 |
| 6 | TREML1 | triggering receptor expressed on myeloid cells like 1 | 161911 | 001167 | 095970 | 32, |
| 6 | TREML2 | triggering receptor expressed on myeloid cells like 2 | 112195 | 095970 | 188056 | 41, 32, 5 |
| 6 | TREML4 | triggering receptor expressed on myeloid cells like 4 | 188056 | 112195 | 124731 | 8, |
| 6 | TRIM39- | TRIM39-RPP21 readthrough | 248167 | 204599 | 241370 | |
| | RPP21 | | | | | |
| 6 | TRIM10 | tripartite motif containing 10 | 204613 | 204614 | 204610 | 19, 5, 6 |
| 6 | TRIM15 | tripartite motif containing 15 | 204610 | 204613 | 234127 | 16, 13, 37 |
| 6 | TRIM26 | tripartite motif containing 26 | 234127 | 204610 | 204599 | 43, 41, 18 |
| 6 | TRIM27 | tripartite motif containing 27 | 204713 | 232040 | 197935 | 21, 3, 29 |
| 6 | TRIM31 | tripartite motif containing 31 | 204616 | 204618 | 204614 | 16, 4, 3 |
| 6 | TRIM38 | tripartite motif containing 38 | 112343 | 112337 | 124610 | 1, 22, 30 |
| 6 | TRIM39 | tripartite motif containing 39 | 204599 | 234127 | 248167 | 40, 38, 9 |
| 6 | TRIM40 | tripartite motif containing 40 | 204614 | 204616 | 204613 | 32, 16, 12 |
| 6 | TRMT11 | tRNA methyltransferase 11 homolog | 066651 | 111911 | 203760 | 11, 34, 29 |
| 6 | TPBG | trophoblast glycoprotein | 146242 | 005700 | 118420 | 11, 45, 12 |
| 6 | TSPYL1 | TSPY like 1 | 189241 | 111817 | 188820 | 11, 29, 6 |
| 6 | TSPYL4 | TSPY like 4 | 187189 | 123500 | 111817 | 8, 11, 31 |
| 6 | TTK | TTK protein kinase | 112742 | 281602 | 083123 | 39, 38, 13 |
| 6 | TULP1 | tubby like protein 1 | 112041 | 007866 | 096060 | 14, 12, 25 |
| 6 | TULP4 | tubby like protein 4 | 130338 | 272047 | 146433 | 18, 35, 1 |
| 6 | TUBB2A | tubulin beta 2A class IIa | 137267 | 137274 | 137285 | 26, 27, 21 |
| 6 | TUBB2B | tubulin beta 2B class IIb | 137285 | 137267 | 180822 | 8, 26, 12 |
| 6 | TUBB | tubulin beta class I | 196230 | 137337 | 137312 | 21, 12, 35 |
| 6 | TUBE1 | tubulin epsilon 1 | 074935 | 112761 | 203778 | 11, 38, 1 |
| 6 | TBCC | tubulin folding cofactorC | 124659 | 112619 | 112624 | 34, 28, 20 |
| 6 | TTLL2 | tubulin tyrosine ligase like 2 | 120440 | 112494 | 203690 | 38, |
| 6 | TINAG | tubulointerstitial nephritis antigen | 137251 | 146147 | 168143 | 17, 37, 3 |
| 6 | TDRD6 | tudor domain containing 6 | 180113 | 153291 | 146070 | 31, 38, 10 |
| 6 | TNF | tumor necrosis factor | 232810 | 226979 | 227507 | 5, 22, 20 |
| 6 | TPD52L1 | tumor protein D52-like 1 | 111907 | 146373 | 111906 | 23, 33, 34 |
| 6 | TDP2 | tyrosyl-DNA phosphodiesterase 2 | 111802 | 137261 | 112304 | 13, 4, 9 |
| 6 | UQCC2 | ubiquinol-cytochrome c reductase complex assembly factor 2 | 137288 | 096433 | 161896 | 6, 15, 22 |
| 6 | UBE2J1 | ubiquitin conjugating enzyme E2 J1 | 198833 | 111886 | 025039 | 22, 41, 45 |
| 6 | UBD | ubiquitin D | 213886 | 204687 | 204681 | 30, 4, 37 |
| 6 | UBR2 | ubiquitin protein ligase E3 component n-recognin 2 | 024048 | 124496 | 112619 | 31, 24, 42 |
| 6 | UBE3D | ubiquitin protein ligase E3D | 118420 | 146242 | 083097 | 29, 21, 9 |
| 6 | USP45 | ubiquitin specific peptidase 45 | 123552 | 132424 | 228439 | 6, 34, 29 |
| 6 | USP49 | ubiquitin specific peptidase 49 | 164663 | 214736 | 124641 | 14, 24, 12 |
| 6 | UFL1 | UFM1 specific ligase 1 | 014123 | 172461 | 112214 | 18, 22, 11 |
| 6 | UHRF1BP1 | UHRF1 binding protein 1 | 065060 | 124562 | 064995 | 23, 3, 43 |
| 6 | ULBP1 | UL16 binding protein 1 | 111981 | 131015 | 155918 | 9, 28, 6 |
| 6 | ULBP2 | UL16 binding protein 2 | 131015 | 203722 | 111981 | 6, 24, 22 |
| 6 | ULBP3 | UL16 binding protein 3 | 131019 | 155918 | 198729 | 3, 40, 45 |
| 6 | UNC5CL | unc-5 family C-terminal like | 124602 | 156564 | 124596 | 17, 28, 5 |
| 6 | UNC93A | unc-93 homolog A (C. elegans) | 112494 | 120436 | 120440 | 37, 19, 15 |
| 6 | UST | uranyl 2-sulfotransferase | 111962 | 111961 | 055208 | 42, 1, 11 |
| 6 | UTRN | utrophin | 152818 | 135604 | 112425 | 23, 29, 1 |
| 6 | VARS | valyl-tRNA synthetase | 204394 | 204396 | 204392 | 21, 35, 27 |
| 6 | VARS2 | valyl-tRNA synthetase 2, mitochondrial | 137411 | 213780 | 196260 | 21, 42, 8 |
| 6 | VNN1 | vanin 1 | 112299 | 146399 | 093134 | |
| 6 | VNN2 | vanin 2 | 112303 | 093134 | 146409 | |
| 6 | VNN3 | vanin 3 | 093134 | 112299 | 112303 | 42, 5, 13 |
| 6 | VEGFA | vascular endothelial growth factor A | 112715 | 096080 | 181577 | 40, 1, 19 |
| 6 | VIP | vasoactive intestinal peptide | 146469 | 120279 | 112029 | 26, 12, 8 |
| 6 | VTA1 | vesicle trafficking 1 | 009844 | 203733 | 112414 | 7, 30, 4 |
| 6 | VGLL2 | vestigial like family member 2 | 170162 | 185002 | 047936 | 25, 19, 32 |
| 6 | VWA7 | von Willebrand factor A domain containing 7 | 204396 | 228727 | 204394 | 42, 26, 37 |
| 6 | VPS52 | VPS52, GARP complex subunit | 223501 | 204227 | 231500 | 39, 24, 35 |
| 6 | WASF1 | WAS protein family member 1 | 112290 | 146360 | 168438 | 38, 8, 26 |
| 6 | WDR27 | WD repeat domain 27 | 184465 | 186340 | 185127 | 22, 39, 10 |
| 6 | WDR46 | WD repeat domain 46 | 227057 | 235863 | 204220 | 21, 13, 36 |
| 6 | WRNIP1 | Werner helicase interacting protein 1 | 124535 | 145949 | 021355 | 29, 22, 20 |
| 6 | WTAP | Wilms tumor 1 associated protein | 146457 | 112096 | 120437 | 37, 29, 24 |
| 6 | WISP3 | WNT1 inducible signaling pathway protein 3 | 112761 | 010810 | 074935 | 17, |
| 6 | YIPF3 | Yip1 domain family member 3 | 137207 | 171453 | 124571 | 27, 32, 41 |
| 6 | ZFP57 | ZFP57 zinc finger protein | 204644 | 204655 | 204642 | 32, 23, 44 |
| 6 | ZFAND3 | zinc finger AN1-type containing 3 | 156639 | 112139 | 183826 | 33, 26, 22 |
| 6 | ZBTB12 | zinc finger and BTB domain containing 12 | 204366 | 166278 | 244255 | 16, 12, |
| 6 | ZBTB2 | zinc finger and BTB domain containing 2 | 181472 | 131016 | 155906 | 40, 7, 43 |
| 6 | ZBTB22 | zinc finger and BTB domain containing 22 | 236104 | 231925 | 204209 | 30, 20, 14 |
| 6 | ZBTB24 | zinc finger and BTB domain containing 24 | 112365 | 135596 | 155085 | 4, 23, 9 |
| 6 | ZBTB9 | zinc finger and BTB domain containing 9 | 213588 | 197283 | 030110 | 18, 14, 24 |
| 6 | ZSCAN12 | zinc finger and SCAN domain containing 12 | 158691 | 189298 | 187987 | 18, 11, 13 |
| 6 | ZSCAN16 | zinc finger and SCAN domain containing 16 | 196812 | 197279 | 198315 | 1, 30, 22 |
| 6 | ZSCAN23 | zinc finger and SCAN domain containing 23 | 187987 | 158691 | 198704 | 25, 8, 38 |
| 6 | ZSCAN26 | zinc finger and SCAN domain containing 26 | 197062 | 189134 | 276302 | 36, 44, 30 |
| 6 | ZSCAN31 | zinc finger and SCAN domain containing 31 | 235109 | 137338 | 189298 | |
| 6 | ZSCAN9 | zinc finger and SCAN domain containing 9 | 137185 | 198315 | 187626 | 1, 37, 26 |
| 6 | ZBED9 | zinc finger BED-type containing 9 | 232040 | 224586 | 204713 | 38, 25, 12 |
| 6 | ZC2HC1B | zinc finger C2HC-type containing 1B | 118491 | 280148 | 118495 | 23, 38, 30 |
| 6 | ZC3H12D | zinc finger CCCH-type containing 12D | 178199 | 177688 | 131013 | 41, 22, 36 |
| 6 | ZDHHC14 | zinc finger DHHC-type containing 14 | 175048 | 215712 | 130340 | 14, 22, 18 |
| 6 | ZNF165 | zinc finger protein 165 | 197279 | 124657 | 196812 | 38, 3, 27 |
| 6 | ZNF184 | zinc finger protein 184 | 096654 | 124613 | 185130 | 22, 1, 11 |
| 6 | ZNF292 | zinc finger protein 292 | 188994 | 135346 | 164411 | |
| 6 | ZNF311 | zinc finger protein 311 | 197935 | 204713 | 204704 | 18, 32, 28 |
| 6 | ZNF318 | zinc finger protein 318 | 171467 | 146215 | 124574 | 22, 13, 21 |
| 6 | ZNF322 | zinc finger protein 322 | 181315 | 146109 | 124635 | 43, 11, 3 |
| 6 | ZNF391 | zinc finger protein 391 | 124613 | 158553 | 096654 | 42, 38, 19 |
| 6 | ZNF451 | zinc finger protein 451 | 112200 | 168116 | 112208 | 26, 11, 9 |
| 6 | ZNF76 | zinc finger protein 76 | 065029 | 146197 | 023892 | 41, 31, 32 |
| 6 | ZKSCAN3 | zinc finger with KRAB and SCAN domains 3 | 189298 | 235109 | 158691 | 15, 9, 45 |
| 6 | ZKSCAN4 | zinc finger with KRAB and SCAN domains 4 | 187626 | 137185 | 189134 | 9, 5, 41 |
| 6 | ZKSCAN8 | zinc finger with KRAB and SCAN domains 8 | 198315 | 196812 | 137185 | 42, 39, 1 |
| 6 | ZUFSP | zinc finger with UFM1 specific peptidase domain | 153975 | 111834 | 196911 | 45, 26, 7 |
| 6 | ZNRD1 | zinc ribbon domain containing 1 | 066379 | 206503 | 204619 | 7, 35, 2 |

Table 4 provides the gene information for the genes found on Chromosomes 7-9.

TABLE 4
Genes on Chromosomes 7-9

| | | | | Up | Down | Tissue |
|---|---|---|---|---|---|---|
| Chr. | Gene Symbol | Gene Name | ENSG | ENSG | ENSG | Code |
| 7 | HIBADH | 3-hydroxyisobutyrate dehydrogenase | 106049 | 106038 | 106052 | 4, 17, 31 |
| 7 | HTR5A | 5-hydroxytryptamine receptor 5A | 157219 | 157212 | 186480 | 39, 8, 38 |
| 7 | NT5C3A | 5′-nucleotidase, cytosolic IIIA | 122643 | 122642 | 164610 | 25, 4, 22 |
| 7 | ADCK2 | aarF domain containing kinase 2 | 133597 | 146966 | 090266 | 41, 22, 1 |
| 7 | ABHD11 | abhydrolase domain containing 11 | 106077 | 106089 | 165215 | 37, 27, 1 |
| 7 | ACHE | acetylcholinesterase (Cartwright blood group) | 087085 | 176125 | 169894 | 26, 8, 15 |
| 7 | ASIC3 | acid sensing ion channel subunit 3 | 213199 | 197150 | 164885 | 40, 38, 36 |
| 7 | ACTB | actin beta | 075624 | 155034 | 075618 | 43, 13, 41 |
| 7 | ACTL6B | actin like 6B | 077080 | 106327 | 172354 | 31, 28, 8 |
| 7 | ARPC1A | actin related protein 2/3 complex subunit 1A | 241685 | 185467 | 130429 | 9, 11, 35 |
| 7 | ARPC1B | actin related protein 2/3 complex subunit 1B | 130429 | 241685 | 106244 | 34, 41, 21 |
| 7 | AGK | acylglycerol kinase | 006530 | 261115 | 257093 | 35, 4, 26 |
| 7 | AOAH | acyloxyacyl hydrolase | 136250 | 011426 | 155849 | 36, 5, 39 |
| 7 | ADAM22 | ADAM metallopeptidase domain 22 | 008277 | 006634 | 075142 | 6, |
| 7 | AP1S1 | adaptor related protein complex 1 sigma 1 subunit | 106367 | 106366 | 128564 | 3, 26, 23 |
| 7 | AP4M1 | adaptor related protein complex 4 mu 1 subunit | 221838 | 166508 | 106290 | 41, 38, 11 |
| 7 | AP5Z1 | adaptor related protein complex 5 zeta 1 subunit | 242802 | 164916 | 157927 | 21, 3, 26 |
| 7 | ADCYAP1R1 | ADCYAP receptor type I | 078549 | 106128 | 164600 | 26, 8, 31 |
| 7 | AHCYL2 | adenosylhomocysteinase like 2 | 158467 | 128602 | 128578 | 16, 1, 31 |
| 7 | ADCY1 | adenylate cyclase 1 | 164742 | 122679 | 283247 | 14, 8, 15 |
| 7 | ARF5 | ADP ribosylation factor 5 | 004059 | 179562 | 106328 | 34, 13, 41 |
| 7 | ARL4A | ADP ribosylation factor like GTPase 4A | 122644 | 006747 | 006468 | 5, 29, 38 |
| 7 | AEBP1 | AE binding protein 1 | 106624 | 122678 | 106628 | 1, 39, 34 |
| 7 | AKAP9 | A-kinase anchoring protein 9 | 127914 | 127989 | 001630 | 23, 18, 4 |
| 7 | AKR1B1 | aldo-keto reductase family 1 member B | 085662 | 205060 | 198074 | 2, 17, 32 |
| 7 | AKR1B10 | aldo-keto reductase family 1 member B10 | 198074 | 085662 | 227471 | 20, 3, 13 |
| 7 | AKR1B15 | aldo-keto reductase family 1 member B15 | 227471 | 198074 | 172331 | 13, 19, 25 |
| 7 | AKR1D1 | aldo-keto reductase family 1 member D1 | 122787 | 182158 | 122779 | |
| 7 | ALKBH4 | alkB homolog 4, lysine demethylase | 160993 | 160991 | 161036 | 21, 1, 26 |
| 7 | AGMO | alkylglycerol monooxygenase | 187546 | 136267 | 106511 | 10, 45, 17 |
| 7 | AZGP1 | alpha-2-glycoprotein 1, zinc-binding | 160862 | 176402 | 106261 | 19, 33, 23 |
| 7 | AOC1 | amine oxidase, copper containing 1 | 002726 | 002933 | 055118 | |
| 7 | AIMP2 | aminoacyl tRNA synthetase complex interacting multifunctional protein 2 | 106305 | 122512 | 086232 | 21, 1, 34 |
| 7 | AASS | aminoadipate-semialdehyde synthase | 008311 | 106278 | 128610 | 12, 24, 18 |
| 7 | AMPH | amphiphysin | 078053 | 211701 | 006715 | 8, 31, 15 |
| 7 | ANLN | anillin actin binding protein | 011426 | 164542 | 136250 | 13, 8, 45 |
| 7 | ANKIB1 | ankyrin repeat and IBR domain containing 1 | 001629 | 001631 | 157259 | 18, 29, 34 |
| 7 | ANKMY2 | ankyrin repeat and MYND domain containing 2 | 106524 | 205858 | 136261 | 4, 7, 11 |
| 7 | ASB10 | ankyrin repeat and SOCS box containing 10 | 146926 | 164900 | 278685 | 15, 25, |
| 7 | ASB15 | ankyrin repeat and SOCS box containing 15 | 146809 | 128609 | 170807 | 24, 15, 38 |
| 7 | ASB4 | ankyrin repeat and SOCS box containing 4 | 005981 | 105854 | 004799 | 2, 20, 24 |
| 7 | ANKRD61 | ankyrin repeat domain 61 | 157999 | 086232 | 106346 | 16, |
| 7 | ANKRD7 | ankyrin repeat domain 7 | 106013 | 128534 | 184408 | 38, 8, 17 |
| 7 | ASZ1 | ankyrin repeat, SAM and basic leucine zipper domain containing 1 | 154438 | 105989 | 001626 | 38, |
| 7 | AGR2 | anterior gradient 2, protein disulphide isomerase family member | 106541 | 280130 | 173467 | 37, 28, 3 |
| 7 | AGR3 | anterior gradient 3, protein disulphide isomerase family member | 173467 | 106541 | 106546 | 37, 3, 20 |
| 7 | AQP1 | aquaporin 1 (Colton blood group) | 240583 | 250424 | 106128 | 39, 26, 17 |
| 7 | AMZ1 | archaelysin family metallopeptidase 1 | 174945 | 136295 | 146535 | 38, |
| 7 | ADAP1 | ArfGAP with dual PH domains 1 | 105963 | 239857 | 240230 | 16, 22, 1 |
| 7 | AGFG2 | ArfGAP with FG repeats 2 | 106351 | 166924 | 205307 | 26, 11, 36 |
| 7 | AGAP3 | ArfGAP with GTPase domain, ankyrin repeat and PH domain 3 | 133612 | 164897 | 164900 | 26, 40, 11 |
| 7 | ASL | argininosuccinate lyase | 126522 | 169919 | 249319 | 3, 9, 34 |
| 7 | ARMC10 | armadillo repeat containing 10 | 170632 | 128606 | 161048 | |
| 7 | ACTR3B | ARP3 actin related protein 3 homolog B | 133627 | 196584 | 130226 | 3, 29, 9 |
| 7 | ACTR3C | ARP3 actin-related protein 3 homolog C | 106526 | 171130 | 127399 | 26, 29, 40 |
| 7 | AHR | aryl hydrocarbon receptor | 106546 | 173467 | 283321 | 26, 43, 29 |
| 7 | ASNS | asparagine synthetase (glutamine-hydrolyzing) | 070669 | 006128 | 135175 | 21, 34, 9 |
| 7 | ATXN7L1 | ataxin 7 like 1 | 146776 | 185055 | 128536 | 30, 9, 13 |
| 7 | ABCA13 | ATP binding cassette subfamily A member 13 | 179869 | 183696 | 188730 | 42, 5, 24 |
| 7 | ABCB1 | ATP binding cassette subfamily B member 1 | 085563 | 005471 | 105784 | 2, 26, 17 |
| 7 | ABCB4 | ATP binding cassette subfamily B member 4 | 005471 | 005469 | 085563 | |
| 7 | ABCB5 | ATP binding cassette subfamily B member 5 | 004846 | 105855 | 164651 | 35, 23, 16 |
| 7 | ABCB8 | ATP binding cassette subfamily B member 8 | 197150 | 181652 | 213199 | |
| 7 | ABCF2 | ATP binding cassette subfamily F member 2 | 033050 | 278685 | 033100 | 21, 3, 26 |
| 7 | ATP5J2 | ATP synthase, H+ transporting, mitochondrial Fo complex subunit F2 | 241468 | 160917 | 198556 | |
| 7 | AGBL3 | ATP/GTP binding protein like 3 | 146856 | 122786 | 122783 | 39, 38, 3 |
| 7 | ATP5J2- | ATP5J2-PTCD1 readthrough | 248919 | 106246 | 160917 | |
| | PTCD1 | | | | | |
| 7 | ATP6V0A4 | ATPase H+ transporting V0 subunit a4 | 105929 | 157703 | 214128 | |
| 7 | ATP6V0E2 | ATPase H+ transporting V0 subunit e2 | 171130 | 106479 | 106526 | |
| 7 | ATP6V1F | ATPase H+ transporting V1 subunit F | 128524 | 135253 | 272899 | 29, 34, 2 |
| 7 | AUTS2 | autism susceptibility candidate 2 | 158321 | 198874 | 185274 | 11, 26, 18 |
| 7 | ATG9B | autophagy related 9B | 181652 | 164867 | 197150 | |
| 7 | AVL9 | AVL9 cell migration associated | 105778 | 106355 | 170852 | 26, 11, 22 |
| 7 | BAIAP2L1 | BAH associated protein 2 like 1 | 006453 | 164713 | 106236 | 37, 16, 3 |
| 7 | BBS9 | Bardet-Biedl syndrome 9 | 122507 | 164610 | 164619 | 42, 1, 38 |
| 7 | BMT2 | base methyltransferase of 25S rRNA 2 homolog | 164603 | 146802 | 164604 | 11, 7, 6 |
| 7 | BHLHA15 | basic helix-loop-helix family member a15 | 180535 | 164715 | 205356 | 22, |
| 7 | BZW2 | basic leucine zipper and W2 domains 2 | 136261 | 106524 | 106537 | 18, 15, 32 |
| 7 | BLACE | B-cell acute lymphoblastic leukemia expressed | 204960 | 186480 | 164778 | |
| 7 | BCAP29 | B-cell receptor associated protein 29 | 075790 | 105865 | 091137 | 38, 26, 45 |
| 7 | BCL7B | BCL tumor suppressor 7B | 106635 | 009954 | 106638 | 9, 28, 32 |
| 7 | BET1 | Bet1 golgi vesicular membrane trafficking protein | 105829 | 127920 | 164692 | 7, 29, 34 |
| 7 | BLVRA | biliverdin reductase A | 106605 | 106603 | 062582 | 34, 43, 30 |
| 7 | BPGM | bisphosphoglycerate mutase | 172331 | 227471 | 122786 | 32, 5, 43 |
| 7 | BMPER | BMP binding endothelial regulator | 164619 | 122507 | 187258 | 13, 31, 45 |
| 7 | BRAF | B-Raf proto-oncogene, serine/threonine kinase | 157764 | 090266 | 090263 | 1, 22, 31 |
| 7 | BRI3 | brain protein I3 | 164713 | 205356 | 006453 | 20, 16, 17 |
| 7 | BRAT1 | BRCA1 associated ATM activator 1 | 106009 | 106003 | 106012 | 27, 35, 9 |
| 7 | BAZ1B | bromodomain adjacent to zinc finger domain 1B | 009954 | 188763 | 106635 | 23, 43, 18 |
| 7 | BUD31 | BUD31 homolog | 106245 | 106244 | 106246 | 29, 3, 18 |
| 7 | C7orf55- | C7orf55-LUC7L2 readthrough | 269955 | 146963 | 188883 | |
| | LUC7L2 | | | | | |
| 7 | CPED1 | cadherin like and PC-esterase domain containing 1 | 106034 | 071243 | 002745 | 34, 7, 45 |
| 7 | CDHR3 | cadherin related family member 3 | 128536 | 146776 | 008282 | 34, 6, 12 |
| 7 | CALCR | calcitonin receptor | 004948 | 004766 | 127928 | 17, 6, 12 |
| 7 | CADPS2 | calcium dependent secretion activator 2 | 081803 | 128610 | 188050 | 44, 45, 42 |
| 7 | CACNA2D1 | calcium voltage-gated channel auxiliary subunit alpha2delta 1 | 153956 | 019991 | 186472 | 10, 31, 2 |
| 7 | CAMK2B | calcium/calmodulin dependent protein kinase II beta | 058404 | 106636 | 015676 | 8, 26, 22 |
| 7 | CALD1 | caldesmon 1 | 122786 | 172331 | 146856 | 45, 26, 11 |
| 7 | CALN1 | calneuron 1 | 183166 | 185274 | 277149 | 8, 18, 2 |
| 7 | CALU | calumenin | 128595 | 135248 | 128617 | 45, 13, 35 |
| 7 | CREB3L2 | cAMP responsive element binding protein 3 like 2 | 182158 | 279483 | 122787 | 32, 26, 40 |
| 7 | CREB5 | cAMP responsive element binding protein 5 | 146592 | 143814 | 255690 | 5, 36, 10 |
| 7 | CNPY1 | canopy FGF signaling regulator 1 | 146910 | 164778 | 184863 | 38, 8, 23 |
| 7 | CNPY4 | canopy FGF signaling regulator 4 | 166997 | 106290 | 214309 | 15, 34, 35 |
| 7 | CLIP2 | CAP-Gly domain containing linker protein 2 | 106665 | 049541 | 006704 | 34, 43, 3 |
| 7 | CAPZA2 | capping actin protein of muscle Z-line alpha subunit 2 | 198898 | 105976 | 004866 | 4, 7, 30 |
| 7 | CHST12 | carbohydrate sulfotransferase 12 | 136213 | 106263 | 275572 | 26, 36, 27 |
| 7 | CPA1 | carboxypeptidase A1 | 091704 | 158525 | 106477 | 28, 4, 19 |
| 7 | CPA2 | carboxypeptidase A2 | 158516 | 165120 | 128510 | 28, 10, 4 |
| 7 | CPA4 | carboxypeptidase A4 | 128510 | 158516 | 158525 | 17, 3, 24 |
| 7 | CPA5 | carboxypeptidase A5 | 158525 | 128510 | 091704 | 38, 15, 14 |
| 7 | CPVL | carboxypeptidase, vitellogenic like | 106066 | 255690 | 106069 | 30, 32, 40 |
| 7 | CROT | carnitine O-octanoyltransferase | 005469 | 135185 | 005471 | 4, 26, 32 |
| 7 | CASD1 | CAS1 domain containing 1 | 127995 | 164692 | 127990 | 32, 26, 11 |
| 7 | CASP2 | caspase 2 | 106144 | 178826 | 188037 | 22, 18, 39 |
| 7 | CARD11 | caspase recruitment domain family member 11 | 198286 | 146535 | 146555 | 41, 21, 22 |
| 7 | CAV1 | caveolin 1 | 105974 | 105971 | 105976 | 45, 35, 1 |
| 7 | CAV2 | caveolin 2 | 105971 | 281656 | 105974 | 1, 45, 40 |
| 7 | CBLL1 | Cbl proto-oncogene like 1 | 105879 | 091137 | 091138 | 42, 34, 29 |
| 7 | CCL24 | C-C motif chemokine ligand 24 | 106178 | 006606 | 005486 | 36, 19, 16 |
| 7 | CCL26 | C-C motif chemokine ligand 26 | 006606 | 127946 | 106178 | 14, 10, 16 |
| 7 | COM2 | COM2 scaffolding protein | 136280 | 136286 | 136274 | 34, 39, 41 |
| 7 | CNOT4 | CCR4-NOT transcription complex subunit 4 | 080802 | 146857 | 155561 | 29, 22, 4 |
| 7 | CCZ1B | CCZ1 homolog B, vacuolar protein trafficking and biogenesis associated | 146574 | 169402 | 106392 | 23, |
| 7 | CCZ1 | CCZ1 homolog, vacuolar protein trafficking and biogenesis associated | 122674 | 122543 | 155026 | 43, 12, 33 |
| 7 | CD36 | CD36 molecule | 135218 | 127955 | 214415 | 1, 15, 36 |
| 7 | CDCA7L | cell division cycle associated 7 like | 164649 | 105877 | 136237 | 39, 43, 38 |
| 7 | CEP41 | centrosomal protein 41 | 106477 | 091704 | 106484 | 38, 42, 7 |
| 7 | CRCP | CGRP receptor component | 241258 | 249319 | 169902 | 40, 13, 4 |
| 7 | CCT6A | chaperonin containing TCP1 subunit 6A | 146731 | 146733 | 129103 | 21, 35, 12 |
| 7 | CHN2 | chimerin 2 | 106069 | 106066 | 176532 | |
| 7 | CLCN1 | chloride voltage-gated channel 1 | 188037 | 106144 | 159784 | 39, 25, |
| 7 | CHRM2 | cholinergic receptor muscarinic 2 | 181072 | 267697 | 105894 | 15, 25, 20 |
| 7 | CHPF2 | chondroitin polymerizing factor 2 | 033100 | 033050 | 082014 | 3, 34, 7 |
| 7 | CBX3 | chromobox 3 | 122565 | 122566 | 086300 | 43, 11, 4 |
| 7 | C7orf25 | chromosome 7 open reading frame 25 | 136197 | 106571 | 256646 | 2, 29, 45 |
| 7 | C7orf26 | chromosome 7 open reading frame 26 | 146576 | 136247 | 236609 | 22, 1, 6 |
| 7 | C7orf31 | chromosome 7 open reading frame 31 | 153790 | 172115 | 105954 | 38, 36, 20 |
| 7 | C7orf33 | chromosome 7 open reading frame 33 | 170279 | 174469 | 055130 | 14, |
| 7 | C7orf34 | chromosome 7 open reading frame 34 | 165131 | 127412 | 197993 | 38, 10, 5 |
| 7 | C7orf43 | chromosome 7 open reading frame 43 | 146826 | 188186 | 197093 | 21, 5, 38 |
| 7 | C7orf49 | chromosome 7 open reading frame 49 | 122783 | 146856 | 146859 | |
| 7 | C7orf50 | chromosome 7 open reading frame 50 | 146540 | 073067 | 164849 | |
| 7 | C7orf57 | chromosome 7 open reading frame 57 | 164746 | 164744 | 183696 | 18, 38, |
| 7 | C7orf61 | chromosome 7 open reading frame 61 | 185955 | 160813 | 166925 | 38, 10, 27 |
| 7 | C7orf62 | chromosome 7 open reading frame 62 | 164645 | 182348 | 164647 | 38, 8, |
| 7 | C7orf72 | chromosome 7 open reading frame 72 | 164500 | 042813 | 185811 | 38, |
| 7 | C7orf73 | chromosome 7 open reading frame 73 | 243317 | 155561 | 164707 | 34, 7, 4 |
| 7 | C7orf77 | chromosome 7 open reading frame 77 | 275356 | 170775 | 128513 | |
| 7 | CFAP69 | cilia and flagella associated protein 69 | 105792 | 157214 | 283267 | 26, 33, 29 |
| 7 | CLDN12 | claudin 12 | 157224 | 105793 | 058091 | 42, 24, 4 |
| 7 | CLDN15 | claudin 15 | 106404 | 106400 | 214253 | 1, 26, 34 |
| 7 | CLDN3 | claudin 3 | 165215 | 106077 | 189143 | 16, 28, 40 |
| 7 | CLDN4 | claudin 4 | 189143 | 165215 | 165171 | |
| 7 | CPSF4 | cleavage and polyadenylation specific factor 4 | 160917 | 248919 | 241468 | 21, 34, 9 |
| 7 | COPG2 | coatomer protein complex subunit gamma 2 | 158623 | 106484 | 213265 | 18, 39, 14 |
| 7 | CCDC126 | coiled-coil domain containing 126 | 169193 | 164548 | 279448 | 31, 22, 40 |
| 7 | CCDC129 | coiled-coil domain containing 129 | 180347 | 164600 | 106341 | 42, 20, 8 |
| 7 | CCDC136 | coiled-coil domain containing 136 | 128596 | 128617 | 128591 | 38, 14, 8 |
| 7 | CCDC146 | coiled-coil domain containing 146 | 135205 | 146707 | 127951 | 45, 26, 42 |
| 7 | CCDC71L | coiled-coil domain containing 71-like | 253276 | 105835 | 105851 | 18, 16, 43 |
| 7 | CHCHD2 | coiled-coil-helix-coiled-coil-helix domain containing 2 | 106153 | 164776 | 185290 | |
| 7 | CHCHD3 | coiled-coil-helix-coiled-coil-helix domain containing 3 | 106554 | 221866 | 131558 | 25, 15, 10 |
| 7 | COL1A2 | collagen type I alpha 2 chain | 164692 | 105829 | 127995 | 11, 6, 12 |
| 7 | COL26A1 | collagen type XXVI alpha 1 chain | 160963 | 128581 | 106436 | 27, 12, 14 |
| 7 | COL28A1 | collagen type XXVIII alpha 1 chain | 215018 | 106392 | 164654 | 26, 32, 33 |
| 7 | COG5 | component of oligomeric golgi complex 5 | 164597 | 105856 | 172209 | 42, 24, 9 |
| 7 | CNTNAP2 | contactin associated protein-like 2 | 174469 | 196511 | 170279 | 12, 26, 11 |
| 7 | COPS6 | COP9 signalosome subunit 6 | 168090 | 166526 | 166508 | 34, 30, 35 |
| 7 | COBL | cordon-bleu WH2 repeat protein | 106078 | 106070 | 221900 | 9, 29, 11 |
| 7 | C1GALT1 | core 1 synthase, glycoprotein-N-acetylgalactosamine | 106392 | 146574 | 215018 | |
| | | 3-beta-galactosyltransferase 1 | | | | |
| 7 | CTTNBP2 | cortactin binding protein 2 | 077063 | 001626 | 128534 | 1, 5, 8 |
| 7 | CRHR2 | corticotropin releasing hormone receptor 2 | 106113 | 106105 | 241644 | |
| 7 | COX19 | COX19, cytochrome c oxidase assembly factor | 240230 | 105963 | 073067 | 11, 42, 3 |
| 7 | CRYGN | crystallin gamma N | 127377 | 187260 | 106615 | 12, 32, |
| 7 | CTAGE15 | CTAGE family member 15 | 271079 | 176510 | 170379 | 38, 3, 32 |
| 7 | CTAGE4 | CTAGE family member 4 | 225932 | 221938 | 213214 | |
| 7 | CTAGE6 | CTAGE family member 6 | 271321 | 283528 | 198420 | 38, 35, 12 |
| 7 | CTAGE8 | CTAGE family member 8 | 244693 | 243896 | 221970 | |
| 7 | CLEC2L | C-type lectin domain family 2 member L | 236279 | 188883 | 064393 | 15, 2, 39 |
| 7 | CLEC5A | C-type lectin domain family 5 member A | 258227 | 258083 | 257138 | 5, 10, 7 |
| 7 | CUL1 | cullin 1 | 055130 | 170279 | 106462 | 38, 3, 22 |
| 7 | CUX1 | cut like homeobox 1 | 257923 | 279538 | 160999 | 39, 21, 24 |
| 7 | DMTF1 | cyclin D binding myb like transcription factor 1 | 135164 | 164659 | 135185 | 39, 22, 42 |
| 7 | CDK13 | cyclin dependent kinase 13 | 065883 | 006451 | 168303 | 22, 27, 30 |
| 7 | CDK14 | cyclin dependent kinase 14 | 058091 | 157224 | 157240 | |
| 7 | CDK5 | cyclin dependent kinase 5 | 164885 | 213199 | 164889 | 2, 7, 41 |
| 7 | CDK6 | cyclin dependent kinase 6 | 105810 | 234545 | 205413 | 29, 22, 37 |
| 7 | CYP3A7- | CYP3A7-CYP3A51P readthrough | 282301 | 106258 | 160870 | |
| | CYP3A51P | | | | | |
| 7 | CFTR | cystic fibrosis transmembrane conductance regulator | 001626 | 154438 | 077063 | |
| 7 | COA1 | cytochrome c oxidase assembly factor 1 homolog | 106603 | 164543 | 106605 | 2, 3, 34 |
| 7 | CYCS | cytochrome c, somatic | 172115 | 070882 | 153790 | |
| 7 | CYP2W1 | cytochrome P450 family 2 subfamily W member 1 | 073067 | 240230 | 146540 | 2, 13, 3 |
| 7 | CYP3A4 | cytochrome P450 family 3 subfamily A member 4 | 160868 | 160870 | 021461 | 19, 10, 23 |
| 7 | CYP3A43 | cytochrome P450 family 3 subfamily A member 43 | 021461 | 160868 | 244623 | 27, 38, |
| 7 | CYP3A5 | cytochrome P450 family 3 subfamily A member 5 | 106258 | 197037 | 282301 | 19, 16, 17 |
| 7 | CYP3A7 | cytochrome P450 family 3 subfamily A member 7 | 160870 | 282301 | 160868 | 19, 15, 36 |
| 7 | CYP51A1 | cytochrome P450 family 51 subfamily A member 1 | 001630 | 127914 | 240720 | 29, 2, 4 |
| 7 | POR | cytochrome p450 oxidoreductase | 127948 | 005486 | 189077 | |
| 7 | CYTH3 | cytohesin 3 | 008256 | 106346 | 178397 | 32, 6, 44 |
| 7 | DBF4 | DBF4 zinc finger | 006634 | 075303 | 008277 | 38, 22, 33 |
| 7 | DDX56 | DEAD-box helicase 56 | 136271 | 015520 | 158604 | 40, 21, 3 |
| 7 | DOCK4 | dedicator of cytokinesis 4 | 128512 | 173114 | 198839 | 8, 40, 18 |
| 7 | DTX2 | deltex E3 ubiquitin ligase 2 | 091073 | 188372 | 243566 | 24, 38, 39 |
| 7 | DENND2A | DENN domain containing 2A | 146966 | 133606 | 133597 | 34, 31, 15 |
| 7 | DFNA5 | DFNA5, deafness associated tumor suppressor | 105928 | 105926 | 070882 | 32, 31, 17 |
| 7 | DGKB | diacylglycerol kinase beta | 136267 | 006468 | 187546 | 26, 8, 28 |
| 7 | DGKI | diacylglycerol kinase iota | 157680 | 105894 | 279483 | 6, |
| 7 | DAGLB | diacylglycerol lipase beta | 164535 | 136238 | 136240 | 24, 39, 22 |
| 7 | DLD | dihydrolipoamide dehydrogenase | 091140 | 091138 | 091136 | 3, 2, 42 |
| 7 | DUS4L | dihydrouridine synthase 4 like | 105865 | 172209 | 075790 | 43, 36, 6 |
| 7 | DPP6 | dipeptidyl peptidase like 6 | 130226 | 133627 | 157212 | |
| 7 | DLX5 | distal-less homeobox 5 | 105880 | 006377 | 196636 | 26, 6, 32 |
| 7 | DLX6 | distal-less homeobox 6 | 006377 | 127922 | 105880 | 26, 15, 24 |
| 7 | POLD2 | DNA polymerase delta 2, accessory subunit | 106628 | 106624 | 106631 | 34, 3, 21 |
| 7 | POLM | DNA polymerase mu | 122678 | 164708 | 106624 | 39, 36, 3 |
| 7 | DNAJB6 | DnaJ heat shock protein family (Hsp40) member B6 | 105993 | 009335 | 155093 | 35, 24, 45 |
| 7 | DNAJB9 | DnaJ heat shock protein family (Hsp40) member B9 | 128590 | 177683 | 184903 | 32, 17, 42 |
| 7 | DNAJC2 | DnaJ heat shock protein family (Hsp40) member C2 | 105821 | 105819 | 161057 | 19, 9, 42 |
| 7 | DNAJC30 | DnaJ heat shock protein family (Hsp40) member C30 | 176410 | 176428 | 071462 | 29, 18, 45 |
| 7 | DDC | dopa decarboxylase | 132437 | 132436 | 106070 | 17, 14, 3 |
| 7 | DPY19L1 | dpy-19 like 1 | 173852 | 187258 | 164532 | 6, 10, 35 |
| 7 | DBNL | drebrin like | 136279 | 136206 | 164708 | 41, 36, 34 |
| 7 | DNAAF5 | dynein axonemal assembly factor 5 | 164818 | 188191 | 164828 | |
| 7 | DNAH11 | dynein axonemal heavy chain 11 | 105877 | 105866 | 164649 | 29, 13, 40 |
| 7 | DYNC1I1 | dynein cytoplasmic 1 intermediate chain 1 | 158560 | 004799 | 004864 | 8, 11, 26 |
| 7 | EFCAB10 | EF-hand calcium binding domain 10 | 185055 | 135249 | 146776 | 34, 42, 30 |
| 7 | ELN | elastin | 049540 | 175877 | 106683 | 10, 42, 43 |
| 7 | ERV3-1 | Endogenous retrovirus group 3 member 1 | 213462 | 152926 | 280410 | 34, 38, 22 |
| 7 | ERVW-1 | Endogenous retrovirus group W member 1 | 242950 | 157259 | 127980 | |
| 7 | EEPD1 | endonuclease/exonuclease/phosphatase family domain containing 1 | 122547 | 122545 | 164542 | 37, 6, 23 |
| 7 | EN2 | engrailed homeobox 2 | 164778 | 204960 | 146910 | 23, 44, 16 |
| 7 | ELMO1 | engulfment and cell motility 1 | 155849 | 136250 | 187037 | 8, 40, 18 |
| 7 | EZH2 | enhancer of zeste 2 polycomb repressive complex 2 subunit | 106462 | 055130 | 155660 | 39, 22, 3 |
| 7 | EPDR1 | ependymin related 1 | 086289 | 187037 | 086288 | 26, 17, 2 |
| 7 | EPHA1 | EPH receptor A1 | 146904 | 159840 | 185899 | 3, 20, |
| 7 | EPHB4 | EPH receptor B4 | 196411 | 146839 | 146828 | 37, 2, 34 |
| 7 | EPHB6 | EPH receptor B6 | 106123 | 237254 | 165125 | 41, 1, 4 |
| 7 | EGFR | epidermal growth factor receptor | 146648 | 132432 | 132434 | 24, 32, 1 |
| 7 | EPO | erythropoietin | 130427 | 172336 | 146839 | 33, 14, 38 |
| 7 | ETV1 | ETS variant 1 | 006468 | 122644 | 136267 | 42, 8, 2 |
| 7 | EIF2AK1 | eukaryotic translation initiation factor 2 alpha kinase 1 | 086232 | 106305 | 157999 | 4, 27, 16 |
| 7 | EIF3B | eukaryotic translation initiation factor 3 subunit B | 106263 | 106266 | 136213 | 18, 21, 16 |
| 7 | EIF4H | eukaryotic translation initiation factor 4H | 106682 | 106683 | 086730 | 21, 29, 26 |
| 7 | EVX1 | even-skipped homeobox 1 | 106038 | 106031 | 106049 | 38, |
| 7 | EXOC4 | exocyst complex component 4 | 131558 | 106554 | 155530 | |
| 7 | ESYT2 | extended synaptotagmin 2 | 117868 | 146918 | 126870 | 34, 1, 27 |
| 7 | ELFN1 | extracellular leucine rich repeat and fibronectin type III domain containing 1 | 225968 | 157778 | 002822 | 35, 39, 15 |
| 7 | FAM20C | FAM20C, golgi associated secretory pathway kinase | 177706 | 106410 | 197461 | 34, 26, 18 |
| 7 | FAM126A | family with sequence similarity 126 member A | 122591 | 196683 | 122550 | 45, 43, 22 |
| 7 | FAM131B | family with sequence similarity 131 member B | 159784 | 188037 | 159840 | 8, 12, 38 |
| 7 | FAM133B | family with sequence similarity 133 member B | 234545 | 127993 | 105810 | 1, 31, 27 |
| 7 | FAM180A | family with sequence similarity 180 member A | 189320 | 164707 | 105887 | 10, 6, 45 |
| 7 | FAM185A | family with sequence similarity 185 member A | 222011 | 279168 | 161040 | 7, 27, 25 |
| 7 | FAM188B | family with sequence similarity 188 member B | 106125 | 254959 | 250424 | 26, 42, 40 |
| 7 | FAM200A | family with sequence similarity 200 member A | 221909 | 196652 | 197343 | 4, 2, 15 |
| 7 | FAM220A | family with sequence similarity 220 member A | 178397 | 008256 | 136238 | 22, 13, 38 |
| 7 | FAM221A | family with sequence similarity 221 member A | 188732 | 279448 | 196335 | 38, 28, 45 |
| 7 | FAM3C | family with sequence similarity 3 member C | 196937 | 002745 | 106278 | 40, 35, 22 |
| 7 | FAM71F1 | family with sequence similarity 71 member F1 | 135248 | 283368 | 128595 | 38, 23, 12 |
| 7 | FAM71F2 | family with sequence similarity 71 member F2 | 205085 | 165055 | 283368 | 38, 44, 10 |
| 7 | FASTK | Fas activated serine/threonine kinase | 164896 | 164889 | 164897 | 41, 3, 34 |
| 7 | FSCN1 | fascin actin-bundling protein 1 | 075618 | 075624 | 011275 | 12, 24, 34 |
| 7 | FSCN3 | fascin actin-bundling protein 3 | 106328 | 004059 | 106331 | 38, 44, 17 |
| 7 | FBXL13 | F-box and leucine rich repeat protein 13 | 161040 | 222011 | 128606 | 38, 7, 27 |
| 7 | FBXL18 | F-box and leucine rich repeat protein 18 | 155034 | 182095 | 075624 | |
| 7 | FBXO24 | F-box protein 24 | 106336 | 077454 | 106333 | 38, 22, 5 |
| 7 | FERD3L | Fer3 like bHLH transcription factor | 146618 | 122691 | 105849 | |
| 7 | FEZF1 | FEZ family zinc finger 1 | 128610 | 008311 | 081803 | 18, 3, 12 |
| 7 | FGL2 | fibrinogen like 2 | 127951 | 135205 | 186088 | 30, 45, 5 |
| 7 | FIGNL1 | fidgetin like 1 | 132436 | 185811 | 132437 | 39, 9, 27 |
| 7 | FLNC | filamin C | 128591 | 128596 | 135253 | 25, 28, 18 |
| 7 | FIS1 | fission, mitochondrial 1 | 214253 | 106404 | 128581 | 29, 1, 43 |
| 7 | FKBP14 | FK506 binding protein 14 | 106080 | 136193 | 106086 | 45, 43, 15 |
| 7 | FKBP6 | FK506 binding protein 6 | 077800 | 146755 | 188763 | 38, 27, 5 |
| 7 | FKBP9 | FK506 binding protein 9 | 122642 | 170852 | 122643 | 1, 11, 6 |
| 7 | FOXK1 | forkhead box K1 | 164916 | 146555 | 242802 | 22, 34, 38 |
| 7 | FOXP2 | forkhead box P2 | 128573 | 154415 | 135272 | 11, 24, 26 |
| 7 | FMC1 | formation of mitochondrial complex V assembly factor 1 homolog | 164898 | 157741 | 146963 | 15, 25, 20 |
| 7 | FZD1 | frizzled class receptor 1 | 157240 | 058091 | 127989 | 26, 3, 18 |
| 7 | FZD9 | frizzled class receptor 9 | 188763 | 077800 | 009954 | 35, 20, 38 |
| 7 | GNA12 | G protein subunit alpha 12 | 146535 | 174945 | 198286 | 34, 31, 35 |
| 7 | GNAI1 | G protein subunit alpha H | 127955 | 187391 | 135218 | 1, 42, 8 |
| 7 | GNAT3 | G protein subunit alpha transducin 3 | 214415 | 135218 | 075223 | 20, |
| 7 | GNB2 | G protein subunit beta 2 | 172354 | 077080 | 146830 | 34, 21, 3 |
| 7 | GNG11 | G protein subunit gamma 11 | 127920 | 105825 | 105829 | 43, 1, 25 |
| 7 | GNGT1 | G protein subunit gamma transducin 1 | 127928 | 004948 | 105825 | 32, 14, 45 |
| 7 | GPER1 | G protein-coupled estrogen receptor 1 | 164850 | 164849 | 178381 | 26, 2, 32 |
| 7 | GPR141 | G protein-coupled receptor 141 | 187037 | 155849 | 086289 | 5, |
| 7 | GPR146 | G protein-coupled receptor 146 | 164849 | 146540 | 164850 | 1, 17, 40 |
| 7 | GPR22 | G protein-coupled receptor 22 | 172209 | 164597 | 105865 | 8, 15, 2 |
| 7 | GPR37 | G protein-coupled receptor 37 | 170775 | 234224 | 275356 | 26, 10, 8 |
| 7 | GPR85 | G protein-coupled receptor 85 | 164604 | 164603 | 214194 | 8, 6, 38 |
| 7 | GAL3ST4 | galactose-3-O-sulfotransferase 4 | 197093 | 146826 | 213420 | 39, 13, 24 |
| 7 | GRIFIN | galectin-related inter-fiber protein | 275572 | 136213 | 106003 | |
| 7 | GGCT | gamma-glutamylcyclotransferase | 006625 | 106100 | 281039 | 34, 4, 35 |
| 7 | GSAP | gamma-secretase activating protein | 186088 | 127951 | 127947 | 22, 31, 40 |
| 7 | GJC3 | gap junction protein gamma 3 | 176402 | 146833 | 160862 | 23, 12, |
| 7 | GBX1 | gastrulation brain homeobox 1 | 164900 | 133612 | 146926 | 38, |
| 7 | GATAD1 | GATA zinc finger domain containing 1 | 157259 | 001629 | 242950 | 29, 18, 44 |
| 7 | GATSL2 | GATS protein like 2 | 274070 | 196275 | 274523 | 38, |
| 7 | GATS | GATS, stromal antigen 3 opposite strand | 239521 | 066923 | 213413 | 26, 31, 8 |
| 7 | GTF2I | general transcription factor IIi | 263001 | 006704 | 158517 | 42, 24, 18 |
| 7 | GIMAP1- | GIMAP1-GIMAP5 readthrough | 281887 | 213203 | 196329 | |
| | GIMAP5 | | | | | |
| 7 | GLI3 | GLI family zinc finger 3 | 106571 | 122641 | 136197 | 11, 1, 12 |
| 7 | GBAS | glioblastoma amplified sequence | 146729 | 178665 | 239789 | 15, 25, 30 |
| 7 | GLCCI1 | glucocorticoid induced 1 | 106415 | 219545 | 003147 | 22, 11, 9 |
| 7 | GCK | glucokinase | 106633 | 106631 | 106636 | 31, 1, 28 |
| 7 | GUSB | glucuronidase beta | 169919 | 196715 | 126522 | 36, 3, 42 |
| 7 | GRM3 | glutamate metabotropic receptor 3 | 198822 | 153993 | 164659 | 8, 45, 36 |
| 7 | GRM8 | glutamate metabotropic receptor 8 | 179603 | 128513 | 048405 | |
| 7 | GSTK1 | glutathione S-transferase kappa 1 | 197448 | 221937 | 178826 | 30, 27, 36 |
| 7 | GPNMB | glycoprotein nmb | 136235 | 136243 | 156928 | 24, 5, 13 |
| 7 | GARS | glycyl-tRNA synthetase | 106105 | 281039 | 106113 | 3, 45, 9 |
| 7 | GPC2 | glypican 2 | 213420 | 197093 | 066923 | 12, 22, 38 |
| 7 | GET4 | golgi to ER traffic protein 4 | 239857 | 164828 | 105963 | 37, 21, 31 |
| 7 | GIGYF1 | GRB10 interacting GYF protein 1 | 146830 | 172354 | 172336 | 29, 26, 40 |
| 7 | GRID2IP | Grid2 interacting protein | 215045 | 136240 | 136247 | 22, 25, |
| 7 | GCC1 | GRIP and coiled-coil domain containing 1 | 179562 | 048405 | 004059 | |
| 7 | GRB10 | growth factor receptor bound protein 10 | 106070 | 132437 | 106078 | |
| 7 | GHRHR | growth hormone releasing hormone receptor | 106128 | 240583 | 078549 | 31, 42, 20 |
| 7 | GTF2IRD1 | GTF2I repeat domain containing 1 | 006704 | 106665 | 263001 | 29, 9, 27 |
| 7 | GTF2IRD2 | GTF2I repeat domain containing 2 | 196275 | 158517 | 274070 | 31, 18, 28 |
| 7 | GTF2IRD2B | GTF2I repeat domain containing 2B | 174428 | 274523 | 178809 | 42, 28, 21 |
| 7 | GTPBP10 | GTP binding protein 10 | 105793 | 283267 | 157224 | |
| 7 | GIMAP1 | GTPase, IMAP family member 1 | 213203 | 106560 | 281887 | 30, 36, 5 |
| 7 | GIMAP2 | GTPase, IMAP family member 2 | 106560 | 133561 | 213203 | 34, 30, 39 |
| 7 | GIMAP4 | GTPase, IMAP family member 4 | 133574 | 179144 | 133561 | 5, 30, 43 |
| 7 | GIMAP5 | GTPase, IMAP family member 5 | 196329 | 281887 | 106565 | 30, 36, 5 |
| 7 | GIMAP6 | GTPase, IMAP family member 6 | 133561 | 133574 | 106560 | 45, 42, 39 |
| 7 | GIMAP7 | GTPase, IMAP family member 7 | 179144 | 171115 | 133574 | 30, 29, 17 |
| 7 | GIMAP8 | GTPase, IMAP family member 8 | 171115 | 196456 | 179144 | 1, 42, 36 |
| 7 | H2AFV | H2A histone family member V | 105968 | 196262 | 146676 | 41, 11, 21 |
| 7 | HSPB1 | heat shock protein family B (small) member 1 | 106211 | 177679 | 170027 | 3, 25, 27 |
| 7 | HECW1 | HECT, C2 and WW domain containing E3 ubiquitin protein ligase 1 | 002746 | 106591 | 164543 | 6, 38, |
| 7 | HEPACAM2 | HEPACAM family member 2 | 188175 | 177409 | 004766 | 3, 28, 31 |
| 7 | HGF | hepatocyte growth factor | 019991 | 075223 | 153956 | 32, 7, 23 |
| 7 | HERPUD2 | HERPUD family member 2 | 122557 | 164532 | 122545 | 31, 36, 38 |
| 7 | HNRNPA2B1 | heterogeneous nuclear ribonucleoprotein A2/B1 | 122566 | 050344 | 122565 | 11, 31, 40 |
| 7 | HDAC9 | histone deacetylase 9 | 048052 | 229937 | 122691 | |
| 7 | HBP1 | HMG-box transcription factor 1 | 105856 | 005249 | 164597 | 1, 42, 11 |
| 7 | HOXA1 | homeobox A1 | 105991 | 005020 | 105996 | 38, 4, 3 |
| 7 | HOXA10 | homeobox A10 | 253293 | 257184 | 005073 | 44, 34, 7 |
| 7 | HOXA11 | homeobox A11 | 005073 | 253293 | 106031 | 9, 44, 6 |
| 7 | HOXA13 | homeobox A13 | 106031 | 005073 | 106038 | 4, 2, 33 |
| 7 | HOW | homeobox A2 | 105996 | 105991 | 105997 | 18, 15, 17 |
| 7 | HOXA3 | homeobox A3 | 105997 | 105996 | 197576 | 31, 45, 38 |
| 7 | HOW | homeobox A4 | 197576 | 105997 | 106004 | 37, 6, 16 |
| 7 | HOXA5 | homeobox A5 | 106004 | 197576 | 106006 | 22, 20, 27 |
| 7 | HOXA6 | homeobox A6 | 106006 | 106004 | 122592 | 17, 20, |
| 7 | HOXA7 | homeobox A7 | 122592 | 106006 | 078399 | 16, |
| 7 | HOXA9 | homeobox A9 | 078399 | 122592 | 257184 | |
| 7 | HIPK2 | homeodomain interacting protein kinase 2 | 064393 | 236279 | 059377 | 18, 8, 31 |
| 7 | HIP1 | huntingtin interacting protein 1 | 127946 | 170092 | 006606 | 38, 27, 12 |
| 7 | HUS1 | HUS1 checkpoint clamp component | 136273 | 136205 | 158683 | 4, 29, 36 |
| 7 | HYAL4 | hyaluronoglucosaminidase 4 | 106302 | 106299 | 106304 | 32, |
| 7 | HILPDA | hypoxia inducible lipid droplet associated | 135245 | 279916 | 165055 | 37, 27, 1 |
| 7 | IKZF1 | IKAROS family zinc finger 1 | 185811 | 164500 | 132436 | |
| 7 | INMT | indolethylamine N-methyltransferase | 241644 | 106113 | 254959 | 1, 40, 33 |
| 7 | INHBA | inhibin beta A subunit | 122641 | 175600 | 106571 | |
| 7 | ING3 | inhibitor of growth family member 3 | 071243 | 106025 | 106034 | 34, 4, 11 |
| 7 | INMT- | INMT-FAM188B readthrough (NMD candidate) | 254959 | 241644 | 106125 | 26, 42, 40 |
| | FAM188B | | | | | |
| 7 | IMMP2L | inner mitochondrial membrane peptidase subunit 2 | 184903 | 128590 | 173114 | 10, 34, 7 |
| 7 | IMPDH1 | inosine monophosphate dehydrogenase 1 | 106348 | 224940 | 279916 | 1, 43, 3 |
| 7 | INSIG1 | insulin induced gene 1 | 186480 | 157219 | 204960 | 31, 21, 19 |
| 7 | IGF2BP3 | insulin like growth factor 2 mRNA binding protein 3 | 136231 | 156928 | 164548 | 12, 7, 21 |
| 7 | IGFBP1 | insulin like growth factor binding protein 1 | 146678 | 283247 | 146674 | 32, 19, 44 |
| 7 | IGFBP3 | insulin like growth factor binding protein 3 | 146674 | 146678 | 136205 | 34, 10, 32 |
| 7 | INTS1 | integrator complex subunit 1 | 164880 | 164877 | 198517 | 22, 1, 21 |
| 7 | ITGB8 | integrin subunit beta 8 | 105855 | 183742 | 004846 | |
| 7 | IRF5 | interferon regulatory factor 5 | 128604 | 272899 | 064419 | 22, 5, 39 |
| 7 | IFRD1 | interferon related developmental regulator 1 | 006652 | 198839 | 181016 | 12, |
| 7 | IL6 | interleukin 6 | 136244 | 105889 | 281889 | 20, 10, 28 |
| 7 | IFT22 | intraflagellar transport 22 | 128581 | 214253 | 160963 | |
| 7 | IQUB | IQ motif and ubiquitin domain containing | 164675 | 081800 | 128609 | 38, 42, 36 |
| 7 | IQCE | IQ motif containing E | 106012 | 106009 | 136295 | 35, 3, 38 |
| 7 | IQCA1L | IQ motif containing with AAA domain 1 like | 278685 | 146926 | 033050 | |
| 7 | ICA1 | islet cell autoantigen 1 | 003147 | 106415 | 122584 | 3, 16, 29 |
| 7 | ISPD | isoprenoid synthase domain containing | 214960 | 106511 | 171243 | 5, 25, 32 |
| 7 | JAZF1 | JAZF zinc finger 1 | 153814 | 106052 | 146592 | 2, 45, 1 |
| 7 | KPNA7 | karyopherin subunit alpha 7 | 185467 | 198742 | 241685 | 18, 28, |
| 7 | KDELR2 | KDEL endoplasmic reticulum protein retention receptor 2 | 136240 | 164535 | 215045 | 45, 10, 24 |
| 7 | KLHDC10 | kelch domain containing 10 | 128607 | 091732 | 146842 | 29, 26, 1 1 |
| 7 | KLHL7 | kelch like family member 7 | 122550 | 122591 | 136243 | 31, 38, 13 |
| 7 | KBTBD2 | kelch repeat and BTB domain containing 2 | 170852 | 105778 | 122642 | 43, 3, 11 |
| 7 | KEL | Kell blood group, metallo-endopeptidase | 197993 | 165131 | 179468 | 36, 7, 38 |
| 7 | KIAA0895 | KIAA0895 | 164542 | 122547 | 011426 | |
| 7 | KIAA1147 | KIAA1147 | 257093 | 006530 | 214102 | 29, 18, 20 |
| 7 | KIAA1324L | KIAA1324 like | 164659 | 198822 | 135164 | 24, 1, 12 |
| 7 | KIAA1549 | KIAA1549 | 122778 | 214128 | 146858 | 17, 9, 25 |
| 7 | KCP | kielin/chordin-like protein | 135253 | 128591 | 128524 | 1, 13, 33 |
| 7 | KLRG2 | killer cell lectin like receptor G2 | 188883 | 269955 | 236279 | 12, 39, 15 |
| 7 | KRBA1 | KRAB-A domain containing 1 | 133619 | 181220 | 181444 | 27, 24, 28 |
| 7 | KRIT1 | KRIT1, ankyrin repeat containing | 001631 | 240720 | 001629 | 24, 4, 36 |
| 7 | KLF14 | Kruppel like factor 14 | 266265 | 213265 | 128585 | 38, 35, |
| 7 | LAMB1 | laminin subunit beta 1 | 091136 | 091140 | 091128 | 11, 1, 26 |
| 7 | LAMB4 | laminin subunit beta 4 | 091128 | 091136 | 091129 | 15, 32, 38 |
| 7 | LANCL2 | LanC like 2 | 132434 | 146648 | 154978 | 34, 9, 24 |
| 7 | LAMTOR4 | late endosomal/lysosomal adaptor, MAPK and MTOR activator 4 | 188186 | 214309 | 146826 | 29, 3, 7 |
| 7 | LMOD2 | leiomodin 2 | 170807 | 146809 | 106299 | 25, 15, 24 |
| 7 | LMTK2 | lemur tyrosine kinase 2 | 164715 | 135175 | 180535 | 29, 22, 4 |
| 7 | LEP | leptin | 174697 | 128594 | 106344 | 1, 32, 23 |
| 7 | LRRC17 | leucine rich repeat containing 17 | 128606 | 161040 | 170632 | 11, 45, 15 |
| 7 | LRRC4 | leucine rich repeat containing 4 | 128594 | 197157 | 174697 | 13, 45, 24 |
| 7 | LRRC61 | leucine rich repeat containing 61 | 127399 | 106526 | 188707 | 9, 2, 27 |
| 7 | LRRC72 | leucine rich repeat containing 72 | 205858 | 171243 | 106524 | 38, 10, |
| 7 | LRRN3 | leucine rich repeat neuronal 3 | 173114 | 184903 | 128512 | 8, 38, 15 |
| 7 | LRCH4 | leucine rich repeats and calponin homology domain containing 4 | 077454 | 205307 | 106336 | 36, 5, 39 |
| 7 | LRRD1 | leucine rich repeats and death domain containing 1 | 240720 | 001630 | 001631 | 38, 8, |
| 7 | LRGUK | leucine rich repeats and guanylate kinase domain containing | 155530 | 131558 | 205060 | 37, 42, 20 |
| 7 | LRWD1 | leucine rich repeats and WD repeat domain containing 1 | 161036 | 160993 | 005075 | 38, 41, 21 |
| 7 | LSMEM1 | leucine rich single-pass membrane protein 1 | 181016 | 006652 | 146802 | 26, 25, 15 |
| 7 | LUZP6 | leucine zipper protein 6 | 267697 | 105887 | 181072 | |
| 7 | LFNG | LFNG O-fucosylpeptide 3-beta-N-acetylglucosaminyltransferase | 106003 | 275572 | 106009 | 17, 3, 30 |
| 7 | LIMK1 | LIM domain kinase 1 | 106683 | 049540 | 106682 | 40, 26, 22 |
| 7 | LMBR1 | limb development membrane protein 1 | 105983 | 105982 | 146909 | 45, 14, 38 |
| 7 | LAT2 | linker for activation of T-cells family member 2 | 086730 | 106682 | 049541 | 41, 21, 5 |
| 7 | LHFPL3 | lipoma HMGIC fusion partner-like 3 | 187416 | 164815 | 005483 | 8, 19, 17 |
| 7 | LINC00998 | Long intergenic non-protein coding RNA 998 | 214194 | 164604 | 154415 | |
| 7 | LSM5 | LSM5 homolog, U6 small nuclear RNA and mRNA degradation associated | 106355 | 154678 | 105778 | 11, 7, 15 |
| 7 | LSM8 | LSM8 homolog, U6 small nuclear RNA associated | 128534 | 077063 | 106013 | |
| 7 | LUC7L2 | LUC7 like 2, pre-mRNA splicing factor | 146963 | 164898 | 269955 | 42, 4, 40 |
| 7 | KDM7A | lysine demethylase 7A | 006459 | 059378 | 157800 | 18, 11, 29 |
| 7 | KMT2C | lysine methyltransferase 2C | 055609 | 178234 | 196584 | 18, 26, 23 |
| 7 | KMT2E | lysine methyltransferase 2E | 005483 | 187416 | 135250 | 11, 23, 7 |
| 7 | MACC1 | MACC1, MET transcriptional regulator | 183742 | 173452 | 105855 | 16, 37, 31 |
| 7 | MAD1L1 | MAD1 mitotic arrest deficient like 1 | 002822 | 225968 | 122687 | 10, 35, 41 |
| 7 | MAFK | MAF bZIP transcription factor K | 198517 | 164880 | 164855 | 34, 6, 20 |
| 7 | MKRN1 | makorin ring finger protein 1 | 133606 | 146955 | 146966 | 38, 2, 11 |
| 7 | MDH2 | malate dehydrogenase 2 | 146701 | 127952 | 177679 | 25, 1, 21 |
| 7 | MGAM | maltase-glucoamylase | 257335 | 165076 | 258083 | 17, 16, 5 |
| 7 | MGAM2 | maltase-glucoamylase 2 (putative) | 257743 | 257138 | 258223 | |
| 7 | MTURN | maturin, neural progenitor differentiation regulator homolog | 180354 | 106086 | 180233 | 26, 8, 14 |
| 7 | MIOS | meiosis regulator for oocyte development | 164654 | 215018 | 106399 | 22, 15, 42 |
| 7 | MAGI2 | membrane associated guanylate kinase, WW and PDZ domain containing 2 | 187391 | 006576 | 127955 | |
| 7 | MPP6 | membrane palmitoylated protein 6 | 105926 | 122585 | 105928 | 11, 19, 3 |
| 7 | MEOX2 | mesenchyme homeobox 2 | 106511 | 187546 | 214960 | 1, 10, 11 |
| 7 | MEST | mesoderm specific transcript | 106484 | 106477 | 158623 | 15, 32, 13 |
| 7 | MET | MET proto-oncogene, receptor tyrosine kinase | 105976 | 105974 | 198898 | 3, 13, 42 |
| 7 | MBLAC1 | metallo-beta-lactamase domain containing 1 | 214309 | 166997 | 188186 | 1, 16, 27 |
| 7 | MEPCE | methylphosphate capping enzyme | 146834 | 078487 | 160813 | 40, 2, 18 |
| 7 | METTL2B | methyltransferase like 2B | 165055 | 135245 | 205085 | 21, 9, 23 |
| 7 | MICALL2 | MICAL like 2 | 164877 | 164853 | 164880 | 26, 2, 36 |
| 7 | MCM7 | minichromosome maintenance complex component 7 | 166508 | 168090 | 221838 | 39, 21, 3 |
| 7 | MALSU1 | mitochondrial assembly of ribosomal large subunit 1 | 156928 | 136235 | 136231 | 41, 15, 36 |
| 7 | MRPL32 | mitochondrial ribosomal protein L32 | 106591 | 106588 | 002746 | 31, 1, 3 |
| 7 | MRPS17 | mitochondrial ribosomal protein S17 | 239789 | 146729 | 146733 | 2, 13, 25 |
| 7 | MRPS24 | mitochondrial ribosomal protein S24 | 062582 | 106605 | 106608 | 15, 25, 41 |
| 7 | MRPS33 | mitochondrial ribosomal protein S33 | 090263 | 157764 | 261115 | 29, 15, 30 |
| 7 | MRM2 | mitochondrial rRNA methyltransferase 2 | 122687 | 002822 | 106268 | |
| 7 | MTERF1 | mitochondrial transcription termination factor 1 | 127989 | 157240 | 127914 | 30, 5, 31 |
| 7 | MLXIPL | MLX interacting protein like | 009950 | 106638 | 176428 | 37, 16, 1 |
| 7 | MOGAT3 | monoacylglycerol O-acyltransferase 3 | 106384 | 167011 | 106397 | 16, |
| 7 | MMD2 | monocyte to macrophage differentiation associated 2 | 136297 | 218823 | 272968 | 8, 38, 14 |
| 7 | MOSPD3 | motile sperm domain containing 3 | 106330 | 106333 | 106327 | 6, 34, 29 |
| 7 | MNX1 | motor neuron and pancreas homeobox 1 | 130675 | 146909 | 009335 | 28, 16, 3 |
| 7 | MPLKIP | M-phase specific PLK1 interacting protein | 168303 | 065883 | 175600 | 31, 26, 2 |
| 7 | MTRNR2L6 | MT-RNR2-like 6 | 270672 | 211750 | 282499 | 44, |
| 7 | MUC12 | mucin 12, cell surface associated | 205277 | 169894 | 169876 | 16, 40, 27 |
| 7 | MUC17 | mucin 17, cell surface associated | 169876 | 205277 | 169871 | 37, 16, 28 |
| 7 | MUC3A | mucin 3A, cell surface associated | 169894 | 087085 | 205277 | |
| 7 | MKLN1 | muskelin 1 | 128585 | 266265 | 128567 | 26, 29, 17 |
| 7 | MDFIC | MyoD family inhibitor domain containing | 135272 | 128573 | 105967 | 22, 4, 11 |
| 7 | MYO1G | myosin IG | 136286 | 146676 | 136280 | 41, 39, 21 |
| 7 | MYL10 | myosin light chain 10 | 106436 | 160963 | 279538 | |
| 7 | MYL7 | myosin light chain 7 | 106631 | 106628 | 106633 | 15, 20, 39 |
| 7 | MTPN | myotrophin | 105887 | 189320 | 267697 | 9, 7, 23 |
| 7 | NACAD | NAC alpha domain containing | 136274 | 136280 | 136270 | 14, 27, 33 |
| 7 | NAT16 | N-acetyltransferase 16 (putative) | 167011 | 128564 | 106384 | 14, 20, 8 |
| 7 | NAPEPLD | N-acyl phosphatidylethanolamine phospholipase D | 161048 | 170632 | 105819 | 7, 29, 19 |
| 7 | NDUFA5 | NADH: ubiquinone oxidoreductase subunit A5 | 128609 | 164675 | 146809 | 7, 34, 4 |
| 7 | NDUFB2 | NADH: ubiquinone oxidoreductase subunit B2 | 090266 | 133597 | 157764 | 43, 25, 40 |
| 7 | NDUFA4 | NDUFA4, mitochondrial complex associated | 189043 | 122584 | 106443 | 29, 15, 45 |
| 7 | NUB1 | negative regulator of ubiquitin like proteins 1 | 013374 | 082014 | 187260 | 30, 31, 40 |
| 7 | NXPH1 | neurexophilin 1 | 122584 | 003147 | 189043 | 2, 8, 23 |
| 7 | NRCAM | neuronal cell adhesion molecule | 091129 | 091128 | 135241 | 26, 43, 8 |
| 7 | NEUROD6 | neuronal differentiation 6 | 164600 | 078549 | 180347 | 8, 45, 25 |
| 7 | NPTX2 | neuronal pentraxin 2 | 106236 | 006453 | 166448 | 2, 31, 28 |
| 7 | NYAP1 | neuronal tyrosine phosphorylated phosphoinositide-3-kinase adaptor 1 | 166924 | 166925 | 106351 | 26, 31, 28 |
| 7 | NPSR1 | neuropeptide S receptor 1 | 187258 | 164619 | 173852 | 3, 14, 38 |
| 7 | NPVF | neuropeptide VF precursor | 105954 | 153790 | 050344 | 14, 10, |
| 7 | NPY | neuropeptide Y | 122585 | 196335 | 105926 | 26, 33, 28 |
| 7 | NCF1 | neutrophil cytosolic factor 1 | 158517 | 263001 | 196275 | 22, 21, 41 |
| 7 | NAMPT | nicotinamide phosphoribosyltransferase | 105835 | 008282 | 253276 | 5, 13, 36 |
| 7 | NOS3 | nitric oxide synthase 3 | 164867 | 055118 | 181652 | 36, 3, 37 |
| 7 | NME8 | NME/NM23 family member 8 | 086288 | 086289 | 106483 | 38, |
| 7 | NOBOX | NOBOX oogenesis homeobox | 106410 | 050327 | 196511 | |
| 7 | NCAPG2 | non-SMC condensin II complex subunit G2 | 146918 | 155093 | 117868 | 39, 10, 38 |
| 7 | NSUN5 | NOP2/Sun RNA methyltransferase family member 5 | 130305 | 155428 | 146755 | 21, 36, 3 |
| 7 | NPC1L1 | NPC1 like intracellular cholesterol transporter 1 | 015520 | 015676 | 136271 | 28, 19, 14 |
| 7 | NFE2L3 | nuclear factor, erythroid 2 like 3 | 050344 | 105954 | 122566 | 26, 3, 16 |
| 7 | NUPR2 | nuclear protein 2, transcriptional regulator | 185290 | 106153 | 268187 | |
| 7 | NRF1 | nuclear respiratory factor 1 | 106459 | 240204 | 186591 | 3, 26, 22 |
| 7 | NOM1 | nucleolar protein with MIF4G domain 1 | 146909 | 105983 | 130675 | 21, 30, 12 |
| 7 | NUP205 | nucleoporin 205 | 155561 | 080802 | 243317 | 24, 31, 37 |
| 7 | NUPL2 | nucleoporin like 2 | 136243 | 122550 | 136235 | 4, 26, 11 |
| 7 | NOD1 | nucleotide binding oligomerization domain containing 1 | 106100 | 281593 | 006625 | 26, 1, 36 |
| 7 | NUDCD3 | NudC domain containing 3 | 015676 | 058404 | 015520 | 25, 26, 9 |
| 7 | NUDT1 | nudix hydrolase 1 | 106268 | 122687 | 106266 | 21, 18, 7 |
| 7 | OR10AC1 | olfactory receptor family 10 subfamily AC member 1 (gene/pseudogene) | 176510 | 221855 | 271079 | |
| 7 | OR2A1 | olfactory receptor family 2 subfamily A member 1 | 221970 | 244693 | 050327 | 27, |
| 7 | OR2A12 | olfactory receptor family 2 subfamily A member 12 | 221858 | 221933 | 221989 | |
| 7 | OR2A14 | olfactory receptor family 2 subfamily A member 14 | 221938 | 221989 | 225932 | |
| 7 | OR2A2 | olfactory receptor family 2 subfamily A member 2 | 221989 | 221858 | 221938 | |
| 7 | OR2A25 | olfactory receptor family 2 subfamily A member 25 | 221933 | 221836 | 221858 | |
| 7 | OR2A42 | olfactory receptor family 2 subfamily A member 42 | 212807 | 213214 | 243896 | |
| 7 | OR2A5 | olfactory receptor family 2 subfamily A member 5 | 221836 | 221813 | 221933 | |
| 7 | OR2A7 | olfactory receptor family 2 subfamily A member 7 | 243896 | 212807 | 244693 | 40, 16, 5 |
| 7 | OR2AE1 | olfactory receptor family 2 subfamily AE member 1 | 244623 | 021461 | 146833 | |
| 7 | OR2F1 | olfactory receptor family 2 subfamily F member 1 (gene/pseudogene) | 213215 | 221910 | 221813 | |
| 7 | OR2F2 | olfactory receptor family 2 subfamily F member 2 | 221910 | 198420 | 213215 | |
| 7 | OR6B1 | olfactory receptor family 6 subfamily B member 1 | 221813 | 213215 | 221836 | |
| 7 | OR6V1 | olfactory receptor family 6 subfamily V member 1 | 225781 | 179468 | 159763 | |
| 7 | OR9A2 | olfactory receptor family 9 subfamily A member 2 | 179468 | 197993 | 225781 | |
| 7 | OR9A4 | olfactory receptor family 9 subfamily A member 4 | 258083 | 257335 | 258227 | 32, |
| 7 | OCM | oncomodulin | 122543 | 011275 | 122674 | 17, 38, |
| 7 | OCM2 | oncomodulin 2 | 135175 | 070669 | 164715 | 10, |
| 7 | OPN1SW | opsin 1 (cone pigments), short-wave-sensitive | 128617 | 128595 | 128596 | 6, 23, 10 |
| 7 | ORAI2 | ORAI calcium release-activated calcium modulator 2 | 160991 | 128563 | 160993 | |
| 7 | ORC5 | origin recognition complex subunit 5 | 164815 | 189056 | 187416 | 38, 9, 31 |
| 7 | OGDH | oxoglutarate dehydrogenase | 105953 | 158604 | 122515 | 43, 42, 24 |
| 7 | OSBPL3 | oxysterol binding protein like 3 | 070882 | 105928 | 172115 | 18, 42, 22 |
| 7 | PAX4 | paired box 4 | 106331 | 106328 | 197157 | |
| 7 | PILRA | paired immunoglobin like type 2 receptor alpha | 085514 | 121716 | 078487 | 23, 5, 20 |
| 7 | PILRB | paired immunoglobin-like type 2 receptor beta | 121716 | 214300 | 085514 | 26, 18, 40 |
| 7 | PON1 | paraoxonase 1 | 005421 | 236197 | 105852 | 19, 6, 23 |
| 7 | PON2 | paraoxonase 2 | 105854 | 105852 | 005981 | 31, 32, 43 |
| 7 | PON3 | paraoxonase 3 | 105852 | 005421 | 105854 | 19, 28, 10 |
| 7 | PNPLA8 | patatin like phospholipase domain containing 8 | 135241 | 091129 | 177683 | 39, 45, 24 |
| 7 | PEG10 | paternally expressed 10 | 242265 | 127990 | 158528 | 31, 32, 15 |
| 7 | PAXIP1 | PAX interacting protein 1 | 157212 | 130226 | 157219 | 37, 39, 9 |
| 7 | PDAP1 | PDGFA associated protein 1 | 106244 | 130429 | 106245 | 3, 26, 27 |
| 7 | PTCD1 | pentatricopeptide repeat domain 1 | 106246 | 106245 | 248919 | 43, 1, 27 |
| 7 | PMPCB | peptidase, mitochondrial processing beta subunit | 105819 | 161048 | 105821 | 3, 2, 34 |
| 7 | PPIA | peptidylprolyl isomerase A | 196262 | 122515 | 105968 | 43, 41, 21 |
| 7 | PEX1 | peroxisomal biogenesis factor 1 | 127980 | 242950 | 127993 | 22, 23, 15 |
| 7 | PHF14 | PHD finger protein 14 | 106443 | 189043 | 005108 | 10, 26, 6 |
| 7 | PIK3CG | phosphatidylinositol-4,5-bisphosphate 3-kinase catalytic subunit gamma | 105851 | 253276 | 005249 | |
| 7 | PDE1C | phosphodiesterase 1C | 154678 | 106341 | 106355 | 26, 8, 15 |
| 7 | PGAM2 | phosphoglycerate mutase 2 | 164708 | 136279 | 122678 | 25, 15, 20 |
| 7 | PRPS1L1 | phosphoribosyl pyrophosphate synthetase 1-like 1 | 229937 | 071189 | 048052 | 38, 8, |
| 7 | PHKG1 | phosphorylase kinase catalytic subunit gamma 1 | 164776 | 129103 | 106153 | |
| 7 | PSPH | phosphoserine phosphatase | 146733 | 239789 | 146731 | 9, 3, 13 |
| 7 | PCLO | piccolo presynaptic cytomatrix protein | 186472 | 153956 | 170381 | 40, 23, 2 |
| 7 | PDGFA | platelet derived growth factor subunit A | 197461 | 177706 | 188191 | 26, 13, 27 |
| 7 | PLEKHA8 | pleckstrin homology domain containing A8 | 106086 | 106080 | 180354 | 12, 31, 32 |
| 7 | PTN | pleiotrophin | 105894 | 181072 | 157680 | 11, 29, 10 |
| 7 | PLXNA4 | plexin A4 | 221866 | 128567 | 106554 | 26, 8, 45 |
| 7 | PMS2 | PMS1 homolog 2, mismatch repair system component | 122512 | 155026 | 106305 | 7, 40, 23 |
| 7 | PODXL | podocalyxin like | 128567 | 128585 | 221866 | 12, 27, 1 |
| 7 | PVRIG | poliovirus receptor related immunoglobulin domain containing | 213413 | 239521 | 214300 | 21, 5, 41 |
| 7 | PAPOLB | poly(A) polymerase beta | 218823 | 157927 | 136297 | 38, 8, 5 |
| 7 | PARP12 | poly(ADP-ribose) polymerase family member 12 | 059378 | 059377 | 006459 | 24, 4, 30 |
| 7 | PKD1L1 | polycystin 1 like 1, transient receptor potential channel interacting | 158683 | 136273 | 164744 | 44, 38, 14 |
| 7 | GALNT11 | polypeptide N-acetylgalactosaminyltransferase 11 | 178234 | 106648 | 055609 | 17, 40, 31 |
| 7 | GALNTL5 | polypeptide N-acetylgalactosaminyltransferase-like 5 | 106648 | 106617 | 178234 | 38, 8, |
| 7 | POMZP3 | POM121 and ZP3 fusion | 146707 | 185040 | 135205 | 34, 10, 1 |
| 7 | POM121 | POM121 transmembrane nucleoporin | 196313 | 277149 | 155428 | 22, 3, 35 |
| 7 | POM121C | POM121 transmembrane nucleoporin C | 272391 | 178809 | 170092 | 30, 37, 31 |
| 7 | POM121L12 | POM121 transmembrane nucleoporin like 12 | 221900 | 106078 | 170419 | 38, 28, |
| 7 | POP7 | POP7 homolog, ribonuclease P/MRP subunit | 172336 | 146830 | 130427 | 18, 34, 27 |
| 7 | KCTD7 | potassium channel tetramerization domain containing 7 | 243335 | 169902 | 154710 | 8, 11, 33 |
| 7 | KCND2 | potassium voltage-gated channel subfamily D member 2 | 184408 | 106013 | 106025 | 8, 45, 2 |
| 7 | KCNH2 | potassium voltage-gated channel subfamily H member 2 | 055118 | 002726 | 164867 | 3, 31, 26 |
| 7 | POU6F2 | POU class 6 homeobox 2 | 106536 | 006715 | 241127 | 38, 8, 16 |
| 7 | PRKRIP1 | PRKR interacting protein 1 (IL11 inducible) | 128563 | 260097 | 160991 | 17, 33, 18 |
| 7 | PCOLCE | procollagen C-endopeptidase enhancer | 106333 | 106336 | 106330 | 34, 6, 15 |
| 7 | PLOD3 | procollagen-lysine,2-oxoglutarate 5-dioxygenase 3 | 106397 | 106384 | 106400 | 3, 45, 35 |
| 7 | PIP | prolactin induced protein | 159763 | 225781 | 236398 | 34, 14, 33 |
| 7 | PRR15 | proline rich 15 | 176532 | 106069 | 122574 | 16, 27, 40 |
| 7 | PRRT4 | proline rich transmembrane protein 4 | 224940 | 106344 | 106348 | 12, 14, 23 |
| 7 | PRSS1 | protease, serine 1 | 204983 | 232869 | 275896 | 28, 4, 19 |
| 7 | PRSS2 | protease, serine 2 | 275896 | 204983 | 282431 | |
| 7 | PRSS37 | protease, serine 37 | 165076 | 127366 | 257335 | 26, 10, 38 |
| 7 | PRSS58 | protease, serine 58 | 258223 | 257743 | 226660 | 38, |
| 7 | PSMC2 | proteasome 26S subunit, ATPase 2 | 161057 | 105821 | 170615 | 13, 3, 30 |
| 7 | PSMG3 | proteasome assembly chaperone 3 | 157778 | 164855 | 225968 | 43, 6, 3 |
| 7 | PSMA2 | proteasome subunit alpha 2 | 106588 | 256646 | 106591 | 29, 30, 3 |
| 7 | POT1 | protection of telomeres 1 | 128513 | 275356 | 179603 | 9, 26, 31 |
| 7 | PDIA4 | protein disulfide isomerase family A member 4 | 155660 | 106462 | 197362 | 40, 37, 13 |
| 7 | PRKAG2 | protein kinase AMP-activated non-catalytic subunit gamma 2 | 106617 | 106615 | 106648 | 43, 26, 29 |
| 7 | PRKAR1B | protein kinase cAMP-dependent type I regulatory subunit beta | 188191 | 197461 | 164818 | 8, 27, 26 |
| 7 | PRKAR2B | protein kinase cAMP-dependent type II regulatory subunit beta | 005249 | 105851 | 105856 | 1, 26, 2 |
| 7 | PPP1R17 | protein phosphatase 1 regulatory subunit 17 | 106341 | 180347 | 154678 | 8, 20, 14 |
| 7 | PPP1R35 | protein phosphatase 1 regulatory subunit 35 | 160813 | 146834 | 185955 | 6, 28, 41 |
| 7 | PPP1R3A | protein phosphatase 1 regulatory subunit 3A | 154415 | 214194 | 128573 | 25, 15, 24 |
| 7 | PPP1R9A | protein phosphatase 1 regulatory subunit 9A | 158528 | 242265 | 236197 | 29, 31, 42 |
| 7 | PTPN12 | protein tyrosine phosphatase, non-receptor type 12 | 127947 | 186088 | 187257 | 26, 11, 1 |
| 7 | PTPRN2 | protein tyrosine phosphatase, receptor type N2 | 155093 | 105993 | 146918 | 31, 8, 28 |
| 7 | PTPRZ1 | protein tyrosine phosphatase, receptor type Z1 | 106278 | 196937 | 008311 | |
| 7 | PUS7 | pseudouridylate synthase 7 (putative) | 091127 | 135250 | 135249 | 4, 24, 30 |
| 7 | PURB | purine rich element binding protein B | 146676 | 105968 | 136286 | 29, 2, 4 |
| 7 | PHTF2 | putative homeodomain transcription factor 2 | 006576 | 135211 | 187391 | 34, 22, 40 |
| 7 | PDK4 | pyruvate dehydrogenase kinase 4 | 004799 | 005981 | 158560 | 42, 2, 25 |
| 7 | RABGEF1 | RAB guanine nucleotide exchange factor 1 | 154710 | 243335 | 106609 | 1, 13, 42 |
| 7 | RAB19 | RAB19, member RAS oncogene family | 146955 | 157800 | 133606 | 40, 23, |
| 7 | RINT1 | RAD50 interactor 1 | 135249 | 091127 | 185055 | 9, 10, 22 |
| 7 | RSPH10B | radial spoke head 10 homolog B | 155026 | 122674 | 122512 | 38, 2, 40 |
| 7 | RSPH10B2 | radial spoke head 10 homolog B2 | 169402 | 164631 | 146574 | |
| 7 | RADIL | Rap associating with DIL domain | 157927 | 242802 | 218823 | 23, 38, 35 |
| 7 | RAPGEF5 | Rap guanine nucleotide exchange factor 5 | 136237 | 164649 | 105889 | 29, 30, 22 |
| 7 | RHEB | Ras homolog enriched in brain | 106615 | 127377 | 106617 | 10, 45, 32 |
| 7 | RALA | RAS like proto-oncogene A | 006451 | 241127 | 065883 | 45, 43, 24 |
| 7 | RASA4 | RAS p21 protein activator 4 | 105808 | 205238 | 205236 | 41, 29, 26 |
| 7 | RASA4B | RAS p21 protein activator 4B | 170667 | 005075 | 168255 | |
| 7 | RAC1 | ras-related C3 botulinum toxin substrate 1 (rho family, small GTP binding protein | 136238 | 178397 | 164535 | 11, 3, 28 |
| | | Rac1) | | | | |
| 7 | RBAK | RB associated KRAB zinc finger | 146587 | 272968 | 157954 | 40, 31, 29 |
| 7 | RBAK- | RBAK-RBAKDN readthrough | 272968 | 136297 | 146587 | |
| | RBAKDN | | | | | |
| 7 | RCC1L | RCC1 like | 274523 | 274070 | 174428 | 34, 21, 27 |
| 7 | RAMP3 | receptor activity modifying protein 3 | 122679 | 136270 | 164742 | 1, 26, 28 |
| 7 | RELN | reelin | 189056 | 170615 | 164815 | 11, 42, 8 |
| 7 | RFC2 | replication factor C subunit 2 | 049541 | 086730 | 106665 | 41, 34, 21 |
| 7 | REPIN1 | replication initiator 1 | 214022 | 106538 | 196456 | 39, 22, 29 |
| 7 | RPA3 | replication protein A3 | 106399 | 164654 | 219545 | 34, 23, 21 |
| 7 | RP9 | retinitis pigmentosa 9 (autosomal dominant) | 164610 | 122643 | 122507 | 27, 22, 15 |
| 7 | RARRES2 | retinoic acid receptor responder 2 | 106538 | 188707 | 214022 | 29, 2, 1 |
| 7 | ARHGEF35 | Rho guanine nucleotide exchange factor 35 | 213214 | 225932 | 212807 | 23, 16, 20 |
| 7 | ARHGEF5 | Rho guanine nucleotide exchange factor 5 | 050327 | 221970 | 106410 | 4, 3, 29 |
| 7 | RHBDD2 | rhomboid domain containing 2 | 005486 | 106178 | 127948 | 34, 26, 31 |
| 7 | RNF133 | ring finger protein 133 | 188050 | 081803 | 235631 | 38, 8, |
| 7 | RNF148 | ring finger protein 148 | 235631 | 188050 | 128519 | 38, 17, 16 |
| 7 | RNF216 | ring finger protein 216 | 011275 | 075618 | 122543 | |
| 7 | RNF32 | ring finger protein 32 | 105982 | 164690 | 105983 | 38, 34, 42 |
| 7 | RBM28 | RNA binding motif protein 28 | 106344 | 174697 | 224940 | 3, 9, 12 |
| 7 | RBM33 | RNA binding motif protein 33 | 184863 | 146910 | 164690 | 39, 31, 22 |
| 7 | RBM48 | RNA binding motif protein 48 | 127993 | 127980 | 234545 | 31, 30, 27 |
| 7 | POLR2J | RNA polymerase II subunit J | 005075 | 161036 | 170667 | 23, 29, 3 |
| 7 | POLR2J2 | RNA polymerase II subunit J2 | 228049 | 173678 | 279168 | 21, 30, 23 |
| 7 | POLR2J3 | RNA polymerase II subunit J3 | 168255 | 170667 | 270249 | |
| 7 | RSBN1L | round spermatid basic protein 1 like | 187257 | 127947 | 135211 | 23, 22, 14 |
| 7 | RUNDC3B | RUN domain containing 3B | 105784 | 085563 | 075303 | 42, 14, 8 |
| 7 | SUN1 | Sad1 and UNC84 domain containing 1 | 164828 | 164818 | 239857 | 42, 31, 38 |
| 7 | SUN3 | Sad1 and UNC84 domain containing 3 | 164744 | 158683 | 164746 | 38, 6, 10 |
| 7 | SGCE | sarcoglycan epsilon | 127990 | 127995 | 242265 | 45, 26, 10 |
| 7 | SBDS | SBDS ribosome assembly guanine nucleotide exchange factor | 126524 | 106609 | 198874 | |
| 7 | SSC4D | scavenger receptor cysteine rich family member with 4 domains | 146700 | 170027 | 188372 | 30, 40, 12 |
| 7 | SOIN | scinderin | 006747 | 226690 | 122644 | 32, 42, 26 |
| 7 | SOSTDC1 | sclerostin domain containing 1 | 171243 | 214960 | 205858 | 3, 11, 30 |
| 7 | SSPO | SCO-spondin | 197558 | 181444 | 106479 | 31, 27, 22 |
| 7 | SEC61G | Sec61 translocon gamma subunit | 132432 | 170419 | 146648 | 2, 7, 43 |
| 7 | SCRN1 | secemin 1 | 136193 | 122574 | 106080 | 29, 8, 22 |
| 7 | SFRP4 | secreted frizzled related protein 4 | 106483 | 086288 | 010270 | 11, 9, 44 |
| 7 | SEMA3A | semaphorin 3A | 075213 | 170381 | 153993 | 14, 8, 12 |
| 7 | SEMA3C | semaphorin 3C | 075223 | 214415 | 019991 | 4, 42, 23 |
| 7 | SEMA3D | semaphprin 3D | 153993 | 075213 | 198822 | |
| 7 | SEMA3E | semaphprin 3E | 170381 | 186472 | 075213 | 42, 30, 10 |
| 7 | SEPT14 | septin 14 | 154997 | 154978 | 249773 | 38, 8, |
| 7 | SEPT7 | septin 7 | 122545 | 122557 | 122547 | 45, 18, 8 |
| 7 | SSMEM1 | serine rich single-pass membrane protein 1 | 165120 | 146842 | 158516 | 38, 14, |
| 7 | SRRM3 | serine/arginine repetitive matrix 3 | 177679 | 146701 | 106211 | 14, 23, 26 |
| 7 | STK17A | serine/threonine kinase 17a | 164543 | 002746 | 106603 | 41, 22, 9 |
| 7 | STK31 | serine/threonine kinase 31 | 196335 | 188732 | 122585 | 38, 10, 40 |
| 7 | STYXL1 | serine/threonine/tyrosine interacting like 1 | 127952 | 189077 | 146701 | 27, 6, 7 |
| 7 | SERPINE1 | serpin family E member 1 | 106366 | 169871 | 106367 | 43, 45, 32 |
| 7 | SRRT | serrate, RNA effector molecule | 087087 | 087077 | 176125 | 21, 22, 41 |
| 7 | SH2B2 | SH2B adapter protein 2 | 160999 | 257923 | 260097 | 21, 22, 25 |
| 7 | SDK1 | sidekick cell adhesion molecule 1 | 146555 | 198286 | 164916 | 10, 36, 15 |
| 7 | SAP25 | Sin3A associated protein 25 | 205307 | 106351 | 077454 | 39, 5, 37 |
| 7 | SSBP1 | single stranded DNA binding protein 1 | 106028 | 214102 | 127362 | |
| 7 | SMURF1 | SMAD specific E3 ubiquitin protein ligase 1 | 198742 | 196367 | 185467 | 38, 42, 1 |
| 7 | SMKR1 | small lysine rich protein 1 | 240204 | 128578 | 106459 | 38, 20, 10 |
| 7 | SMO | smoothened, frizzled class receptor | 128602 | 158457 | 158467 | 12, 11, 10 |
| 7 | SLC12A9 | solute carrier family 12 member 9 | 146828 | 196411 | 087077 | 9, 26, 37 |
| 7 | SLC13A1 | solute carrier family 13 member 1 | 081800 | 128519 | 164675 | 17, 16, 44 |
| 7 | SLC13A4 | solute carrier family 13 member 4 | 164707 | 243317 | 189320 | 32, 8, 4 |
| 7 | SLC25A13 | solute carrier family 25 member 13 | 004864 | 158560 | 127922 | 2, 3, 13 |
| 7 | SLC25A40 | solute carrier family 25 member 40 | 075303 | 105784 | 006634 | 2, 22, 43 |
| 7 | SLC26A3 | solute carrier family 26 member 3 | 091138 | 105879 | 091140 | 16, 33, 37 |
| 7 | SLC26A4 | solute carrier family 26 member 4 | 091137 | 075790 | 105879 | 40, 45, 42 |
| 7 | SLC26A5 | solute carrier family 26 member 5 | 170615 | 161057 | 189056 | 37, 8, |
| 7 | SLC29A4 | solute carrier family 29 member 4 | 164638 | 157954 | 182095 | 34, 3, 14 |
| 7 | SLC35B4 | solute carrier family 35 member B4 | 205060 | 155530 | 085662 | 26, 23, 45 |
| 7 | SLC37A3 | solute carrier family 37 member 3 | 157800 | 006459 | 146955 | |
| 7 | SLC4A2 | solute carrier family 4 member 2 | 164889 | 164885 | 164896 | 41, 9, 28 |
| 7 | SHH | sonic hedgehog | 164690 | 184863 | 105982 | |
| 7 | SRI | sorcin | 075142 | 008277 | 127954 | 8, 3, 16 |
| 7 | SNX10 | sorting nexin 10 | 086300 | 122565 | 005020 | 7, 5, 17 |
| 7 | SNX13 | sorting nexin 13 | 071189 | 283321 | 229937 | 31, 25, 26 |
| 7 | SNX8 | sorting nexin 8 | 106266 | 106268 | 106263 | 29, 35, 22 |
| 7 | SP4 | Sp4 transcription factor | 105866 | 164651 | 105877 | 39, 11, 25 |
| 7 | SP8 | Sp8 transcription factor | 164651 | 004846 | 105866 | 33, 7, 8 |
| 7 | SPDYE1 | speedy/RINGO cell cycle regulator family member E1 | 136206 | 078967 | 136279 | 18, 22, 40 |
| 7 | SPDYE16 | speedy/RINGO cell cycle regulator family member E16 | 185040 | 243566 | 146707 | |
| 7 | SPDYE2 | speedy/RINGO cell cycle regulator family member E2 | 205238 | 270249 | 105808 | 31, 18, 24 |
| 7 | SPDYE2B | speedy/RINGO cell cycle regulator family member E2B | 173678 | 267645 | 228049 | |
| 7 | SPDYE3 | speedy/RINGO cell cycle regulator family member E3 | 214300 | 213413 | 121716 | 18, 43, 9 |
| 7 | SPDYE5 | speedy/RINGO cell cycle regulator family member E5 | 170092 | 272391 | 127946 | 40, 4, 3 |
| 7 | SPDYE6 | speedy/RINGO cell cycle regulator family member E6 | 260097 | 160999 | 128563 | |
| 7 | SPAM1 | sperm adhesion molecule 1 | 106304 | 106302 | 234224 | 38, 10, 8 |
| 7 | SHFM1 | Split hand/foot malformation (ectrodactyly) type 1 | 127922 | 004864 | 006377 | |
| 7 | SKAP2 | src kinase associated phosphoprotein 2 | 005020 | 086300 | 105991 | 4, 41, 38 |
| 7 | SRPK2 | SRSF protein kinase 2 | 135250 | 005483 | 091127 | 18, 8, 38 |
| 7 | SND1 | staphylococcal nuclease and tudor domain containing 1 | 197157 | 106331 | 128594 | 42, 43, 38 |
| 7 | STARD3NL | STARD3 N-terminal like | 010270 | 106483 | 227191 | 29, 26, 23 |
| 7 | STEAP1 | STEAP family member 1 | 164647 | 164645 | 157214 | 11, 33, 6 |
| 7 | STEAP1B | STEAP family member 1B | 105889 | 136237 | 136244 | 10, 30, 45 |
| 7 | STEAP2 | STEAP2 metalloreductase | 157214 | 164647 | 105792 | 33, 26, 16 |
| 7 | STEAP4 | STEAP4 metalloreductase | 127954 | 075142 | 182348 | 30, 32, 29 |
| 7 | SAMD9 | sterile alpha motif domain containing 9 | 205413 | 105810 | 177409 | 24, 30, 34 |
| 7 | SAMD9L | sterile alpha motif domain containing 9 like | 177409 | 205413 | 188175 | 1, 44, 25 |
| 7 | STRA8 | stimulated by retinoic acid 8 | 146857 | 105875 | 080802 | |
| 7 | STRIP2 | striatin interacting protein 2 | 128578 | 158467 | 240204 | 6, 31, 41 |
| 7 | STAG3 | stromal antigen 3 | 066923 | 213420 | 239521 | 38, 22, 41 |
| 7 | SDHAF3 | succinate dehydrogenase complex assembly factor 3 | 196636 | 105880 | 006128 | 4, 30, 10 |
| 7 | SUGCT | succinyl-CoA: glutarate-CoA transferase | 175600 | 168303 | 122641 | 10, 19, 17 |
| 7 | SUMF2 | sulfatase modifying factor 2 | 129103 | 146731 | 164776 | 13, 37, 40 |
| 7 | ST7 | suppression of tumorigenicity 7 | 004866 | 198898 | 105989 | 9, 16, 17 |
| 7 | SVOPL | SVOP like | 157703 | 122779 | 105929 | 9, 27, 25 |
| 7 | SMARCD3 | SWI/SNF related, matrix associated, actin dependent regulator of chromatin, | 082014 | 033100 | 013374 | 15, 26, 27 |
| | | subfamily d, member 3 | | | | |
| 7 | SYPL1 | synaptophysin like 1 | 008282 | 128536 | 105835 | 13, 4, 1 |
| 7 | STX1A | syntaxin 1A | 106089 | 071462 | 106077 | 31, 8, 26 |
| 7 | TRBC1 | T cell receptor beta constant 1 | 211751 | 282780 | 211764 | |
| 7 | TRBC2 | T cell receptor beta constant 2 | 211772 | 282499 | 211752 | |
| 7 | TRBD1 | T cell receptor beta diversity 1 | 282431 | 275896 | 282320 | |
| 7 | TRBJ1-1 | T cell receptor beta joining 1-1 | 282320 | 282431 | 282420 | |
| 7 | TRBJ1-2 | T cell receptor beta joining 1-2 | 282420 | 282320 | 282133 | |
| 7 | TRBJ1-3 | T cell receptor beta joining 1-3 | 282133 | 282420 | 281958 | |
| 7 | TRBJ1-4 | T cell receptor beta joining 1-4 | 281958 | 282133 | 282173 | |
| 7 | TRBJ1-5 | T cell receptor beta joining 1-5 | 282173 | 281958 | 282780 | |
| 7 | TRBJ1-6 | T cell receptor beta joining 1-6 | 282780 | 282173 | 211751 | |
| 7 | TRBJ2-1 | T cell receptor beta joining 2-1 | 211764 | 211751 | 211765 | |
| 7 | TRBJ2-2 | T cell receptor beta joining 2-2 | 211765 | 211764 | 211766 | |
| 7 | TRBJ2-2P | T cell receptor beta joining 2-2P (non-functional) | 211766 | 211765 | 211767 | |
| 7 | TRBJ2-3 | T cell receptor beta joining 2-3 | 211767 | 211766 | 211768 | |
| 7 | TRBJ2-4 | T cell receptor beta joining 2-4 | 211768 | 211767 | 211769 | |
| 7 | TRBJ2-5 | T cell receptor beta joining 2-5 | 211769 | 211768 | 211770 | |
| 7 | TRBJ2-6 | T cell receptor beta joining 2-6 | 211770 | 211769 | 211771 | |
| 7 | TRBJ2-7 | T cell receptor beta joining 2-7 | 211771 | 211770 | 237254 | |
| 7 | TRBV10-1 | T cell receptor beta variable 10-1 (gene/pseudogene) | 211717 | 211716 | 211720 | |
| 7 | TRBV10-2 | T cell receptor beta variable 10-2 | 229769 | 211720 | 211721 | |
| 7 | TRBV10-3 | T cell receptor beta variable 10-3 | 275791 | 276405 | 276597 | |
| 7 | TRBV11-1 | T cell receptor beta variable 11-1 | 211720 | 211717 | 229769 | |
| 7 | TRBV11-3 | T cell receptor beta variable 11-3 | 276597 | 275791 | 274752 | |
| 7 | TRBV12-3 | T cell receptor beta variable 12-3 | 274752 | 276597 | 276953 | |
| 7 | TRBV12-4 | T cell receptor beta variable 12-4 | 276953 | 274752 | 275158 | |
| 7 | TRBV12-5 | T cell receptor beta variable 12-5 | 275158 | 276953 | 275743 | |
| 7 | TRBV13 | T cell receptor beta variable 13 | 276405 | 278030 | 275791 | |
| 7 | TRBV14 | T cell receptor beta variable 14 | 275743 | 275158 | 276819 | |
| 7 | TRBV15 | T cell receptor beta variable 15 | 276819 | 275743 | 275243 | |
| 7 | TRBV16 | T cell receptor beta variable 16 (gene/pseudogene) | 275243 | 276819 | 277880 | |
| 7 | TRBV17 | T cell receptor beta variable 17 (non-functional) | 277880 | 275243 | 276557 | |
| 7 | TRBV18 | T cell receptor beta variable 18 | 276557 | 277880 | 211746 | |
| 7 | TRBV19 | T cell receptor beta variable 19 | 211746 | 276557 | 211747 | 39, 30, 23 |
| 7 | TRBV2 | T cell receptor beta variable 2 | 226660 | 258223 | 237702 | |
| 7 | TRBV20-1 | T cell receptor beta variable 20-1 | 211747 | 211746 | 211749 | |
| 7 | TRBV23-1 | T cell receptor beta variable 23-1 (non-functional) | 211749 | 211747 | 211750 | |
| 7 | TRBV24-1 | T cell receptor beta variable 24-1 | 211750 | 211749 | 270672 | |
| 7 | TRBV25-1 | T cell receptor beta variable 25-1 | 282499 | 270672 | 211772 | |
| 7 | TRBV27 | T cell receptor beta variable 27 | 211752 | 211772 | 211753 | 39, 7, 5 |
| 7 | TRBV28 | T cell receptor beta variable 28 | 211753 | 211752 | 232869 | |
| 7 | TRBV29-1 | T cell receptor beta variable 29-1 | 232869 | 211753 | 204983 | |
| 7 | TRBV30 | T cell receptor beta variable 30 (gene/pseudogene) | 237254 | 211771 | 106123 | |
| 7 | TRBV3-1 | T cell receptor beta variable 3-1 | 237702 | 226660 | 211710 | |
| 7 | TRBV4-1 | T cell receptor beta variable 4-1 | 211710 | 237702 | 211734 | |
| 7 | TRBV4-2 | T cell receptor beta variable 4-2 | 211745 | 211707 | 283063 | |
| 7 | TRBV5-1 | T cell receptor beta variable 5-1 | 211734 | 211710 | 211706 | |
| 7 | TRBV5-3 | T cell receptor beta variable 5-3 (non-functional) | 211715 | 211714 | 211716 | |
| 7 | TRBV5-4 | T cell receptor beta variable 5-4 | 230099 | 253409 | 211724 | |
| 7 | TRBV5-5 | T cell receptor beta variable 5-5 | 211725 | 211724 | 253188 | |
| 7 | TRBV5-6 | T cell receptor beta variable 5-6 | 211728 | 211727 | 253534 | |
| 7 | TRBV5-7 | T cell receptor beta variable 5-7 (non-functional) | 211731 | 253291 | 278030 | |
| 7 | TRBV6-1 | T cell receptor beta variable 6-1 | 211706 | 211734 | 211707 | |
| 7 | TRBV6-2 | T cell receptor beta variable 6-2 (gene/pseudogene) | 283063 | 211745 | 282939 | |
| 7 | TRBV6-4 | T cell receptor beta variable 6-4 | 211713 | 282939 | 211714 | |
| 7 | TRBV6-5 | T cell receptor beta variable 6-5 | 211721 | 229769 | 253409 | |
| 7 | TRBV6-6 | T cell receptor beta variable 6-6 | 211724 | 230099 | 211725 | |
| 7 | TRBV6-7 | T cell receptor beta variable 6-7 (non-functional) | 253188 | 211725 | 211727 | |
| 7 | TRBV6-8 | T cell receptor beta variable 6-8 | 253534 | 211728 | 253291 | |
| 7 | TRBV7-1 | T cell receptor beta variable 7-1 (non-functional) | 211707 | 211706 | 211745 | |
| 7 | TRBV7-2 | T cell receptor beta variable 7-2 | 282939 | 283063 | 211713 | |
| 7 | TRBV7-3 | T cell receptor beta variable 7-3 | 211714 | 211713 | 211715 | |
| 7 | TRBV7-4 | T cell receptor beta variable 7-4 (gene/pseudogene) | 253409 | 211721 | 230099 | |
| 7 | TRBV7-6 | T cell receptor beta variable 7-6 | 211727 | 253188 | 211728 | |
| 7 | TRBV7-7 | T cell receptor beta variable 7-7 | 253291 | 253534 | 211731 | |
| 7 | TRBV7-9 | T cell receptor beta variable 7-9 | 278030 | 211731 | 276405 | |
| 7 | TRBV9 | T cell receptor beta variable 9 | 211716 | 211715 | 211717 | |
| 7 | TRGC1 | T cell receptor gamma constant 1 | 211689 | 211688 | 211690 | |
| 7 | TRGC2 | T cell receptor gamma constant 2 | 227191 | 010270 | 211687 | |
| 7 | TRGJ1 | T cell receptor gamma joining 1 | 211690 | 211689 | 211691 | |
| 7 | TRGJ2 | T cell receptor gamma joining 2 | 211687 | 227191 | 211688 | |
| 7 | TRGJP | T cell receptor gamma joining P | 211691 | 211690 | 211692 | |
| 7 | TRGJP1 | T cell receptor gamma joining P1 | 211692 | 211691 | 211693 | |
| 7 | TRGJP2 | T cell receptor gamma joining P2 | 211688 | 211687 | 211689 | |
| 7 | TRGV1 | T cell receptor gamma variable 1 (non-functional) | 211701 | 233306 | 078053 | |
| 7 | TRGV10 | T cell receptor gamma variable 10 (non-functional) | 211694 | 211693 | 211695 | |
| 7 | TRGV11 | T cell receptor gamma variable 11 (non-functional) | 211693 | 211692 | 211694 | |
| 7 | TRGV2 | T cell receptor gamma variable 2 | 233306 | 211699 | 211701 | |
| 7 | TRGV3 | T cell receptor gamma variable 3 | 211699 | 211698 | 233306 | |
| 7 | TRGV4 | T cell receptor gamma variable 4 | 211698 | 211697 | 211699 | |
| 7 | TRGV5 | T cell receptor gamma variable 5 | 211697 | 211696 | 211698 | |
| 7 | TRGV8 | T cell receptor gamma variable 8 | 211696 | 211695 | 211697 | |
| 7 | TRGV9 | T cell receptor gamma variable 9 | 211695 | 211694 | 211696 | |
| 7 | TAC1 | tachykinin precursor 1 | 006128 | 196636 | 070669 | 26, 36, 8 |
| 7 | TAS2R16 | taste 2 receptor member 16 | 128519 | 235631 | 081800 | |
| 7 | TAS2R3 | taste 2 receptor member 3 | 127362 | 106028 | 127364 | 25, |
| 7 | TAS2R38 | taste 2 receptor member 38 | 257138 | 258227 | 257743 | |
| 7 | TAS2R39 | taste 2 receptor member 39 | 236398 | 159763 | 221937 | |
| 7 | TAS2R4 | taste 2 receptor member 4 | 127364 | 127362 | 127366 | |
| 7 | TAS2R40 | taste 2 receptor member 40 | 221937 | 236398 | 197448 | 32, |
| 7 | TAS2R41 | taste 2 receptor member 41 | 221855 | 185899 | 176510 | |
| 7 | TAS2R5 | taste 2 receptor member 5 | 127366 | 127364 | 165076 | 27, |
| 7 | TAS2R60 | taste 2 receptor member 60 | 185899 | 146904 | 221855 | |
| 7 | TAF6 | TATA-box binding protein associated factor 6 | 106290 | 221838 | 166997 | 34, 40, 13 |
| 7 | TAX1BP1 | Tax1 binding protein 1 | 106052 | 106049 | 153814 | 45, 31, 2 |
| 7 | TBX20 | T-box 20 | 164532 | 173852 | 122557 | 17, |
| 7 | TECPR1 | tectonin beta-propeller repeat containing 1 | 205356 | 180535 | 164713 | 42, 25, 1 |
| 7 | TNS3 | tensin 3 | 136205 | 146674 | 136273 | 18, 40, 32 |
| 7 | TES | testin LIM domain protein | 135269 | 105967 | 281656 | 40, 44, 23 |
| 7 | TSGA13 | testis specific 13 | 213265 | 158623 | 266265 | 38, 8, |
| 7 | TSPAN12 | tetraspanin 12 | 106025 | 184408 | 071243 | 2, 4, 42 |
| 7 | TSPAN13 | tetraspanin 13 | 106537 | 136261 | 280130 | 3, 31, 4 |
| 7 | TSPAN33 | tetraspanin 33 | 158457 | 230626 | 128602 | 22, 1, 17 |
| 7 | TTC26 | tetratricopeptide repeat domain 26 | 105948 | 105939 | 157741 | 42, 38, 30 |
| 7 | THAP5 | THAP domain containing 5 | 177683 | 135241 | 128590 | 7, 34, 29 |
| 7 | TPK1 | thiamin pyrophosphokinase 1 | 196511 | 106410 | 174469 | 38, 32, 15 |
| 7 | THSD7A | thrombospondin type 1 domain containing 7A | 005108 | 106443 | 106460 | 11, 32, 1 |
| 7 | TBXAS1 | thromboxane A synthase 1 | 059377 | 064393 | 059378 | 36, 5, 10 |
| 7 | TRIP6 | thyroid hormone receptor interactor 6 | 087077 | 146828 | 087087 | 9, 3, 27 |
| 7 | TFPI2 | tissue factor pathway inhibitor 2 | 105825 | 127928 | 127920 | 32, 45, 43 |
| 7 | TRIL | TLR4 interactor with leucine rich repeats | 255690 | 146592 | 106066 | 31, 10, 12 |
| 7 | TFEC | transcription factor EC | 105967 | 135272 | 135269 | |
| 7 | TBL2 | transducin beta like 2 | 106638 | 106635 | 009950 | 38, 9, 41 |
| 7 | TFR2 | transferrin receptor 2 | 106327 | 106330 | 077080 | 19, 10, 9 |
| 7 | TRRAP | transformation/transcription domain associated protein | 196367 | 166448 | 198742 | 18, 22, 21 |
| 7 | TRA2A | transformer 2 alpha homolog | 164548 | 136231 | 169193 | 36, 29, 18 |
| 7 | TBRG4 | transforming growth factor beta regulator 4 | 136270 | 136274 | 122679 | 9, 2, 21 |
| 7 | TRPV5 | transient receptor potential cation channel subfamily V member 5 | 127412 | 165125 | 165131 | 5, 20, |
| 7 | TRPV6 | transient receptor potential cation channel subfamily V member 6 | 165125 | 106123 | 127412 | |
| 7 | TOMM7 | translocase of outer mitochondrial membrane 7 | 196683 | 281889 | 122591 | 41, 25, 43 |
| 7 | TMUB1 | transmembrane and ubiquitin like domain containing 1 | 164897 | 164896 | 133612 | 22, 3, 9 |
| 7 | TMED4 | transmembrane p24 trafficking protein 4 | 158604 | 136271 | 105953 | 29, 11, 18 |
| 7 | TMEM106B | transmembrane protein 106B | 106460 | 005108 | 146530 | 22, 31, 4 |
| 7 | TMEM120A | transmembrane protein 120A | 189077 | 127948 | 127952 | 3, 1, 26 |
| 7 | TMEM130 | transmembrane protein 130 | 166448 | 106236 | 196367 | 8, 31, 26 |
| 7 | TMEM139 | transmembrane protein 139 | 178826 | 197448 | 106144 | 17, 32, 16 |
| 7 | TMEM140 | transmembrane protein 140 | 146859 | 122783 | 105875 | 1, 28, 36 |
| 7 | TMEM168 | transmembrane protein 168 | 146802 | 181016 | 164603 | 13, 29, 42 |
| 7 | TMEM176A | transmembrane protein 176A | 002933 | 106565 | 002726 | 27, 13, 28 |
| 7 | TMEM176B | transmembrane protein 176B | 106565 | 196329 | 002933 | 30, 16, 27 |
| 7 | TMEM178B | transmembrane protein 178B | 261115 | 090263 | 006530 | 40, 29, 8 |
| 7 | TMEM184A | transmembrane protein 184A | 164855 | 198517 | 157778 | |
| 7 | TMEM196 | transmembrane protein 196 | 173452 | 105849 | 183742 | 10, 14, 28 |
| 7 | TMEM209 | transmembrane protein 209 | 146842 | 128607 | 165120 | 30, 22, 4 |
| 7 | TMEM213 | transmembrane protein 213 | 214128 | 105929 | 122778 | 17, 42, 23 |
| 7 | TMEM229A | transmembrane protein 229A | 234224 | 106304 | 170775 | 14, 38, 8 |
| 7 | TMEM243 | transmembrane protein 243 | 135185 | 135164 | 005469 | 29, 22, 21 |
| 7 | TMEM248 | transmembrane protein 248 | 106609 | 154710 | 126524 | 6, 31, 29 |
| 7 | TMEM60 | transmembrane protein 60 | 135211 | 187257 | 006576 | 29, 31, 22 |
| 7 | TNPO3 | transportin 3 | 064419 | 128604 | 230626 | 24, 34, 3 |
| 7 | TNRC18 | trinucleotide repeat containing 18 | 182095 | 164638 | 155034 | 18, 29, 3 |
| 7 | TRIM24 | tripartite motif containing 24 | 122779 | 122787 | 157703 | 12, 18, 3 |
| 7 | TRIM4 | tripartite motif containing 4 | 146833 | 244623 | 176402 | 21, 22, 30 |
| 7 | TRIM50 | tripartite motif containing 50 | 146755 | 130305 | 077800 | 29, 4, 17 |
| 7 | TRIM56 | tripartite motif containing 56 | 169871 | 169876 | 106366 | 22, 21, 36 |
| 7 | TRIM73 | tripartite motif containing 73 | 178809 | 174428 | 272391 | |
| 7 | TRIM74 | tripartite motif containing 74 | 155428 | 196313 | 130305 | 3, 24, 8 |
| 7 | TYW1 | tRNA-yW synthesizing protein 1 homolog | 198874 | 126524 | 158321 | 6, 15, 37 |
| 7 | TYW1B | tRNA-yW synthesizing protein 1 homolog B | 277149 | 183166 | 196313 | 39, 44, 38 |
| 7 | TCAF1 | TRPM8 channel associated factor 1 | 198420 | 271321 | 221910 | |
| 7 | TCAF2 | TRPM8 channel associated factor 2 | 170379 | 271079 | 283528 | |
| 7 | TSC22D4 | TSC22 domain family member 4 | 166925 | 185955 | 166924 | 26, 41, 34 |
| 7 | TTYH3 | tweety family member 3 | 136295 | 106012 | 174945 | 34, 35, 12 |
| 7 | TWIST1 | twist family bHLH transcription factor 1 | 122691 | 048052 | 146618 | 6, 18, 44 |
| 7 | TWISTNB | TWIST neighbor | 105849 | 146618 | 173452 | 30, 2, 27 |
| 7 | YWHAG | tyrosine 3-monooxygenase/tryptophan 5-monooxygenase activation protein | 170027 | 106211 | 146700 | 26, 8, 44 |
| | | gamma | | | | |
| 7 | TPST1 | tyrosylprotein sulfotransferase 1 | 169902 | 241258 | 243335 | 38, 6, 32 |
| 7 | UMAD1 | UBAP1-MVB12-associated (UMA) domain containing 1 | 219545 | 106399 | 106415 | 40, 4, 18 |
| 7 | UBN2 | ubinuclein 2 | 157741 | 105948 | 164898 | 22, 40, 28 |
| 7 | UBE2D4 | ubiquitin conjugating enzyme E2 D4 (putative) | 078967 | 106608 | 136206 | 36, 25, 15 |
| 7 | UBE2H | ubiquitin conjugating enzyme E2 H | 186591 | 106459 | 091732 | 29, 26, 27 |
| 7 | UBE3C | ubiquitin protein ligase E3C | 009335 | 130675 | 105993 | 30, 45, 29 |
| 7 | USP42 | ubiquitin specific peptidase 42 | 106346 | 157999 | 008256 | 42, 38, 22 |
| 7 | UFSP1 | UFM1 specific peptidase 1 (inactive) | 176125 | 087087 | 087085 | 2, 27, 5 |
| 7 | UNCX | UNC homeobox | 164853 | 178381 | 164877 | 14, |
| 7 | URGCP | upregulator of cell proliferation | 106608 | 062582 | 078967 | 26, 6, 42 |
| 7 | URGCP- | URGCP-MRPS24 readthrough | 270617 | 106605 | 106608 | |
| | MRPS24 | | | | | |
| 7 | UPP1 | uridine phosphorylase 1 | 183696 | 164746 | 179869 | 35, 32, 37 |
| 7 | UPK3B | uroplakin 3B | 243566 | 091073 | 185040 | 1, 44, 13 |
| 7 | UPK3BL | uroplakin 3B-like | 267368 | 205236 | 267645 | |
| 7 | VIPR2 | vasoactive intestinal peptide receptor 2 | 106018 | 126870 | 106410 | 9, 15, 39 |
| 7 | VOPP1 | vesicular, overexpressed in cancer, prosurvival protein 1 | 154978 | 132434 | 154997 | 18, 43, 29 |
| 7 | VGF | VGF nerve growth factor inducible | 128564 | 106367 | 167011 | 28, 35, 31 |
| 7 | VKORC1L1 | vitamin K epoxide reductase complex subunit 1 like 1 | 196715 | 280427 | 169919 | 21, 11, 3 |
| 7 | VWC2 | von Willebrand factor C domain containing 2 | 188730 | 179869 | 042813 | 10, 44, 14 |
| 7 | VWDE | von Willebrand factor D and EGF domains | 146530 | 106460 | 226690 | 42, 12, 5 |
| 7 | VPS37D | VPS37D, ESCRT-I subunit | 176428 | 009950 | 176410 | 14, 17, 21 |
| 7 | VPS41 | VPS41, HOPS complex subunit | 006715 | 078053 | 106536 | 6, 29, 24 |
| 7 | VPS50 | VPS50, EARP/GARPII complex subunit | 004766 | 188175 | 004948 | 42, 31, 24 |
| 7 | VSTM2A | V-set and transmembrane domain containing 2A | 170419 | 221900 | 132432 | 8, 45, 17 |
| 7 | WIPF3 | WAS/WASL interacting protein family member 3 | 122574 | 176532 | 136193 | 27, 25, 40 |
| 7 | WDR60 | WD repeat domain 60 | 126870 | 117868 | 106018 | 26, 29, 40 |
| 7 | WDR86 | WD repeat domain 86 | 187260 | 013374 | 127377 | 26, 32, 36 |
| 7 | WDR91 | WD repeat domain 91 | 105875 | 146859 | 146857 | 24, 13, 17 |
| 7 | WIPI2 | WD repeat domain, phosphoinositide interacting 2 | 157954 | 146587 | 164638 | 21, 3, 34 |
| 7 | WEE2 | WEE1 homolog 2 | 214102 | 257093 | 106028 | 23, 39, 37 |
| 7 | WBSCR27 | Williams Beuren syndrome chromosome region 27 | 165171 | 189143 | 175877 | 45, 16, 23 |
| 7 | WBSCR17 | Williams-Beuren syndrome chromosome region 17 | 185274 | 158321 | 183166 | 11, 31, 8 |
| 7 | WBSCR22 | Williams-Beuren syndrome chromosome region 22 | 071462 | 176410 | 106089 | 34, 35, 28 |
| 7 | WBSCR28 | Williams-Beuren syndrome chromosome region 28 | 175877 | 165171 | 049540 | 38, |
| 7 | WASL | Wiskott-Aldrich syndrome like | 106299 | 170807 | 106302 | 26, 29, 35 |
| 7 | WNT16 | Wnt family member 16 | 002745 | 106034 | 196937 | 35, 5, 8 |
| 7 | WNT2 | Wnt family member 2 | 105989 | 004866 | 154438 | 32, 34, 10 |
| 7 | XRCC2 | X-ray repair cross complementing 2 | 196584 | 055609 | 133627 | 34, 12, 24 |
| 7 | YAE1D1 | Yae1 domain containing 1 | 241127 | 106536 | 006451 | 37, 7, 40 |
| 7 | YKT6 | YKT6 v-SNARE homolog (S. cerevisiae) | 106636 | 106633 | 058404 | 34, 35, 18 |
| 7 | ZBED6CL | ZBED6 C-terminal like | 188707 | 127399 | 106538 | 17, 41, 22 |
| 7 | ZNRF2 | zinc and ring finger 2 | 180233 | 180354 | 281593 | 40, 1, 23 |
| 7 | ZFAND2A | zinc finger AN 1-type containing 2A | 178381 | 164850 | 164853 | 42, 39, 40 |
| 7 | ZSCAN21 | zinc finger and SCAN domain containing 21 | 166529 | 106261 | 166526 | 34, 11, 24 |
| 7 | ZSCAN25 | zinc finger and SCAN domain containing 25 | 197037 | 272647 | 106258 | 40, 39, 31 |
| 7 | ZC3HC1 | zinc finger C3HC-type containing 1 | 091732 | 186591 | 128607 | 43, 6, 9 |
| 7 | ZC3HAV1 | zinc finger CCCH-type containing, antiviral 1 | 105939 | 146858 | 105948 | 42, 26, 6 |
| 7 | ZC3HAV1L | zinc finger CCCH-type containing, antiviral 1 like | 146858 | 122778 | 105939 | 39, 44, 35 |
| 7 | ZCWPW1 | zinc finger CW-type and PWWP domain containing 1 | 078487 | 085514 | 146834 | 38, 1, 13 |
| 7 | ZDHHC4 | zinc finger DHHC-type containing 4 | 136247 | 215045 | 146576 | 13, 9, 24 |
| 7 | ZNF783 | zinc finger family member 783 | 204946 | 170260 | 196453 | 1, 22, 31 |
| 7 | ZNHIT1 | zinc finger HIT-type containing 1 | 106400 | 106397 | 106404 | 6, 35, 27 |
| 7 | ZMIZ2 | zinc finger MIZ-type containing 2 | 122515 | 105953 | 196262 | 26, 3, 39 |
| 7 | ZNF107 | zinc finger protein 107 | 196247 | 173041 | 197008 | 22, 26, 39 |
| 7 | ZNF117 | zinc finger protein 117 | 152926 | 198039 | 213462 | 32, 43, 11 |
| 7 | ZNF12 | zinc finger protein 12 | 164631 | 205903 | 169402 | 1, 6, 22 |
| 7 | ZNF138 | zinc finger protein 138 | 197008 | 196247 | 198039 | 26, 31, 39 |
| 7 | ZNF212 | zinc finger protein 212 | 170260 | 170265 | 204946 | 3, 24, 36 |
| 7 | ZNF273 | zinc finger protein 273 | 198039 | 197008 | 152926 | 42, 38, 25 |
| 7 | ZNF277 | zinc finger protein 277 | 198839 | 128512 | 006652 | 43, 29, 9 |
| 7 | ZNF282 | zinc finger protein 282 | 170265 | 197024 | 170260 | 9, 34, 43 |
| 7 | ZNF3 | zinc finger protein 3 | 166526 | 166529 | 168090 | 26, 39, 15 |
| 7 | ZNF316 | zinc finger protein 316 | 205903 | 236609 | 164631 | 40, 29, 9 |
| 7 | ZNF394 | zinc finger protein 394 | 160908 | 198556 | 196652 | 36, 41, 5 |
| 7 | ZNF398 | zinc finger protein 398 | 197024 | 204947 | 170265 | 34, 38, 2 |
| 7 | ZNF425 | zinc finger protein 425 | 204947 | 197362 | 197024 | 29, 6, 3 |
| 7 | ZNF467 | zinc finger protein 467 | 181444 | 133619 | 197558 | 20, 14, 31 |
| 7 | ZNF479 | zinc finger protein 479 | 185177 | 268187 | 182111 | 38, |
| 7 | ZNF655 | zinc finger protein 655 | 197343 | 221909 | 272647 | 42, 11, 37 |
| 7 | ZNF679 | zinc finger protein 679 | 197123 | 223614 | 234444 | 38, |
| 7 | ZNF680 | zinc finger protein 680 | 173041 | 234444 | 196247 | 11, 39, 34 |
| 7 | ZNF713 | zinc finger protein 713 | 178665 | 249773 | 146729 | 2, 36, 38 |
| 7 | ZNF716 | zinc finger protein 716 | 182111 | 185177 | 275217 | 38, 32, |
| 7 | ZNF727 | zinc finger protein 727 | 214652 | 275217 | 223614 | 23, |
| 7 | ZNF735 | zinc finger protein 735 | 223614 | 214652 | 197123 | |
| 7 | ZNF736 | zinc finger protein 736 | 234444 | 197123 | 173041 | 5, 38, 39 |
| 7 | ZNF746 | zinc finger protein 746 | 181220 | 196453 | 133619 | 38, 26, 31 |
| 7 | ZNF775 | zinc finger protein 775 | 196456 | 214022 | 171115 | 11, 31, 27 |
| 7 | ZNF777 | zinc finger protein 777 | 196453 | 204946 | 181220 | 34, 16, 31 |
| 7 | ZNF786 | zinc finger protein 786 | 197362 | 155660 | 204947 | 12, 21, 14 |
| 7 | ZNF789 | zinc finger protein 789 | 198556 | 241468 | 160908 | 22, 31, 36 |
| 7 | ZNF800 | zinc finger protein 800 | 048405 | 179603 | 179562 | 22, 45, 4 |
| 7 | ZNF804B | zinc finger protein 804B | 182348 | 127954 | 164645 | 8, |
| 7 | ZNF853 | zinc finger protein 853 | 236609 | 146576 | 205903 | 26, 18, 4 |
| 7 | ZNF862 | zinc finger protein 862 | 106479 | 197558 | 171130 | 33, 26, 39 |
| 7 | ZNF92 | zinc finger protein 92 | 146757 | 280410 | 280427 | 43, 27, 45 |
| 7 | ZKSCAN1 | zinc finger with KRAB and SCAN domains 1 | 106261 | 160862 | 166529 | 24, 42, 22 |
| 7 | ZKSCAN5 | zinc finger with KRAB and SCAN domains 5 | 196652 | 160908 | 221909 | 43, 25, 31 |
| 7 | ZPBP | zona pellucida binding protein | 042813 | 188730 | 164500 | |
| 7 | ZP3 | zona pellucida glycoprotein 3 | 188372 | 146700 | 091073 | 10, 9, 1 |
| 7 | ZAN | zonadhesin (gene/pseudogene) | 146839 | 130427 | 196411 | 38, |
| 7 | ZYX | zyxin | 159840 | 159784 | 146904 | 5, 27, 1 |
| 8 | AGPAT5 | 1-acylglycerol-3-phosphate O-acyltransferase 5 | 155189 | 091879 | 275591 | 26, 32, 38 |
| 8 | DECR1 | 2,4-dienoyl-CoA reductase 1 | 104325 | 104320 | 104327 | 4, 11, 34 |
| 8 | OPLAH | 5-oxoprolinase (ATP-hydrolysing) | 178814 | 186583 | 178896 | 33, 34, 30 |
| 8 | ADCK5 | aarF domain containing kinase 5 | 173137 | 182325 | 071894 | 1, 36, 22 |
| 8 | ABRA | actin binding Rho activating protein | 174429 | 164830 | 154188 | 25, 15, 33 |
| 8 | ARC | activity regulated cytoskeleton associated protein | 198576 | 181790 | 234616 | 2, 20, 6 |
| 8 | ADAMDEC1 | ADAM like decysin 1 | 134028 | 042980 | 069206 | 25, |
| 8 | ADAM18 | ADAM metallopeptidase domain 18 | 168619 | 197140 | 104755 | 38, 10, 4 |
| 8 | ADAM2 | ADAM metallopeptidase domain 2 | 104755 | 168619 | 131203 | 38, 10, 33 |
| 8 | ADAM28 | ADAM metallopeptidase domain 28 | 042980 | 159167 | 134028 | 27, 18, 21 |
| 8 | ADAM32 | ADAM metallopeptidase domain 32 | 197140 | 168615 | 168619 | 38, 10, 1 |
| 8 | ADAM7 | ADAM metallopeptidase domain 7 | 069206 | 134028 | 104722 | 38, 25, 17 |
| 8 | ADAM9 | ADAM metallopeptidase domain 9 | 168615 | 169490 | 197140 | 45, 37, 33 |
| 8 | AP3M2 | adaptor related protein complex 3 mu 2 subunit | 070718 | 083168 | 104368 | |
| 8 | ADCY8 | adenylate cyclase 8 | 155897 | 153317 | 132294 | 40, 5, 38 |
| 8 | ADGRA2 | adhesion G protein-coupled receptor A2 | 020181 | 147471 | 104221 | 15, 26, 20 |
| 8 | ADGRB1 | adhesion G protein-coupled receptor B1 | 181790 | 171045 | 198576 | |
| 8 | ARFGEF1 | ADP ribosylation factor guanine nucleotide exchange factor 1 | 066777 | 104218 | 165078 | 26, 18, 29 |
| 8 | ADRA1A | adrenoceptor alpha 1A | 120907 | 092964 | 015592 | 38, |
| 8 | ADRB3 | adrenoceptor beta 3 | 188778 | 169154 | 187840 | 27, 32, 8 |
| 8 | AARD | alanine and arginine rich domain containing protein | 205002 | 281527 | 164756 | 38, 35, 17 |
| 8 | ADHFE1 | alcohol dehydrogenase, iron containing 1 | 147576 | 179041 | 169085 | 15, 29, 19 |
| 8 | TTPA | alpha tocopherol transfer protein | 137561 | 137563 | 185728 | 23, 17, 38 |
| 8 | ANGPT1 | angiopoietin 1 | 154188 | 174429 | 147655 | 7, 34, 42 |
| 8 | ANGPT2 | angiopoietin 2 | 091879 | 147316 | 155189 | 45, 43, 32 |
| 8 | ANK1 | ankyrin 1 | 029534 | 165066 | 083168 | 25, 5, 27 |
| 8 | ANKRD46 | ankyrin repeat domain 46 | 186106 | 034677 | 174226 | |
| 8 | ANXA13 | annexin A13 | 104537 | 175946 | 176853 | |
| 8 | AZIN1 | antizyme inhibitor 1 | 155096 | 155090 | 155097 | 42, 24, 39 |
| 8 | ASAP1 | ArfGAP with SH3 domain, ankyrin repeat and PH domain 1 | 153317 | 153310 | 155897 | |
| 8 | AGO2 | argonaute 2, RISC catalytic component | 123908 | 104472 | 169398 | 22, 26, 4 |
| 8 | ARMC1 | armadillo repeat containing 1 | 104442 | 172817 | 066855 | 11, 13, 17 |
| 8 | ASH2L | ASH2 like histone lysine methyltransferase complex subunit | 129691 | 187840 | 147465 | 13, 1, 36 |
| 8 | ASPH | aspartate beta-hydroxylase | 198363 | 177182 | 283485 | 3, 1, 6 |
| 8 | ATAD2 | ATPase family, AAA domain containing 2 | 156802 | 165156 | 156795 | 22, 13, 37 |
| 8 | ATP6V0D2 | ATPase H+ transporting V0 subunit d2 | 147614 | 104267 | 147613 | |
| 8 | ATP6V1B2 | ATPase H+ transporting V1 subunit B2 | 147416 | 036565 | 061337 | 26, 8, 38 |
| 8 | ATP6V1C1 | ATPase H+ transporting V1 subunit C1 | 155097 | 155096 | 164929 | 37, 13, 11 |
| 8 | ATP6V1H | ATPase H+ transporting V1 subunit H | 047249 | 082556 | 147509 | 10, 2, 8 |
| 8 | BHLHE22 | basic helix-loop-helix family member e22 | 180828 | 185728 | 172817 | 8, 14, 33 |
| 8 | BAG4 | BCL2 associated athanogene 4 | 156735 | 175324 | 085788 | 29, 9, 42 |
| 8 | BNIP3L | BCL2 interacting protein 3 like | 104765 | 221914 | 240694 | 5, 7, 1 |
| 8 | BLK | BLK proto-oncogene, Src family tyrosine kinase | 136573 | 154319 | 136574 | 21, 41, 36 |
| 8 | BOP1 | block of proliferation 1 | 261236 | 179832 | 260428 | 21, 35, 7 |
| 8 | BMP1 | bone morphogenetic protein 1 | 168487 | 168484 | 168490 | 32, 3, 6 |
| 8 | BAALC | brain and acute leukemia, cytoplasmic | 164929 | 155097 | 164930 | 34, 8, 26 |
| 8 | BRF2 | BRF2, RNA polymerase III transcription initiation factor subunit | 104221 | 020181 | 156675 | 9, 40, 15 |
| 8 | BIN3 | bridging integrator 3 | 147439 | 158941 | 179388 | 1, 35, 9 |
| 8 | C8orf44-SGK3 | C8orf44-SGK3 readthrough | 270024 | 175073 | 213865 | |
| 8 | CDH17 | cadherin 17 | 079112 | 164951 | 164949 | 16, 25, 37 |
| 8 | CALB1 | calbindin 1 | 104327 | 104325 | 180694 | 17, 10, 11 |
| 8 | PKIA | cAMP-dependent protein kinase inhibitor alpha | 171033 | 164751 | 104427 | 15, 25, 29 |
| 8 | CA1 | carbonic anhydrase 1 | 133742 | 185015 | 164879 | 5, 16, 10 |
| 8 | CA13 | carbonic anhydrase 13 | 185015 | 176731 | 133742 | 29, 27, 42 |
| 8 | CA2 | carbonic anhydrase 2 | 104267 | 164879 | 147614 | 5, 16, 17 |
| 8 | CA3 | carbonic anhydrase 3 | 164879 | 133742 | 104267 | 25, 11, 15 |
| 8 | CA8 | carbonic anhydrase 8 | 178538 | 198846 | 104388 | 10, 35, 15 |
| 8 | CPA6 | carboxypeptidase A6 | 165078 | 066777 | 046889 | 7, 14, 33 |
| 8 | CPQ | carboxypeptidase Q | 104324 | 169439 | 180543 | 1, 10, 29 |
| 8 | CTSB | cathepsin B | 164733 | 079459 | 205884 | 40, 6, 10 |
| 8 | CEBPD | CCAAT/enhancer binding protein delta | 221869 | 164808 | 253729 | 17, 11, 20 |
| 8 | CNOT7 | CCR4-NOT transcription complex subunit 7 | 198791 | 104219 | 155975 | 4, 10, 38 |
| 8 | CCAR2 | cell cycle and apoptosis regulator 2 | 158941 | 241852 | 147439 | 39, 42, 34 |
| 8 | CDCA2 | cell division cycle associated 2 | 184661 | 104756 | 221818 | 22, 38, 9 |
| 8 | CSPP1 | centrosome and spindle pole associated protein 1 | 104218 | 121022 | 066777 | 29, 7, 23 |
| 8 | CLN8 | ceroid-lipofuscinosis, neuronal 8 | 182372 | 283494 | 283239 | 1, 36, 27 |
| 8 | CHMP4C | charged multivesicular body protein 4C | 164695 | 104231 | 104497 | 43, 28, 34 |
| 8 | CHMP7 | charged multivesicular body protein 7 | 147457 | 104689 | 104679 | 21, 2, 22 |
| 8 | CHRNA2 | cholinergic receptor nicotinic alpha 2 subunit | 120903 | 120899 | 120915 | 8, 26, 2 |
| 8 | CHRNA6 | cholinergic receptor nicotinic alpha 6 subunit | 147434 | 147432 | 131931 | 21, 35, 25 |
| 8 | CHRNB3 | cholinergic receptor nicotinic beta 3 subunit | 147432 | 176209 | 147434 | 14, 8, |
| 8 | CSGALNACT1 | chondroitin sulfate N-acetylgalactosaminyltransferase 1 | 147408 | 104611 | 104613 | 2, 32, 40 |
| 8 | CHRAC1 | chromatin accessibility complex 1 | 104472 | 167632 | 123908 | 40, 13, 7 |
| 8 | CHD7 | chromodomain helicase DNA binding protein 7 | 171316 | 104388 | 177182 | |
| 8 | C8orf22 | chromosome 8 open reading frame 22 | 168333 | 019549 | 147481 | 25, 10, 15 |
| 8 | C8orf33 | chromosome 8 open reading frame 33 | 182307 | 170631 | 164935 | 9, 23, 3 |
| 8 | C8orf34 | chromosome 8 open reading frame 34 | 165084 | 046889 | 137573 | 11, 31, 8 |
| 8 | C8orf37 | chromosome 8 open reading frame 37 | 156172 | 175895 | 156466 | 29, 42, 14 |
| 8 | C8orf4 | chromosome 8 open reading frame 4 | 176907 | 188676 | 165061 | 19, 9, 20 |
| 8 | C8orf44 | chromosome 8 open reading frame 44 | 213865 | 270024 | 278888 | 31, 13, 18 |
| 8 | C8orf46 | chromosome 8 open reading frame 46 | 169085 | 147576 | 185697 | |
| 8 | C8orf48 | chromosome 8 open reading frame 48 | 164743 | 164741 | 185053 | 6, 18, 4 |
| 8 | C8orf58 | chromosome 8 open reading frame 58 | 241852 | 248235 | 158941 | 27, 5, 44 |
| 8 | C8orf59 | chromosome 8 open reading frame 59 | 176731 | 133740 | 185015 | 30, 31, 33 |
| 8 | C8orf74 | chromosome 8 open reading frame 74 | 171060 | 183638 | 171056 | 38, |
| 8 | C8orf76 | chromosome 8 open reading frame 76 | 189376 | 147689 | 259305 | 6, 22, 15 |
| 8 | C8orf82 | chromosome 8 open reading frame 82 | 213563 | 254402 | 147799 | 33, 19, 14 |
| 8 | C8orf86 | chromosome 8 open reading frame 86 | 196166 | 077782 | 147526 | 8, |
| 8 | C8orf88 | chromosome 8 open reading frame 88 | 253250 | 123119 | 155099 | |
| 8 | C8orf89 | chromosome 8 open reading frame 89 | 274443 | 164764 | 147604 | |
| 8 | CLDN23 | claudin 23 | 253958 | 275342 | 147324 | 29, 22, 18 |
| 8 | CLVS1 | clavesin 1 | 177182 | 171316 | 198363 | 10, 8, 7 |
| 8 | CPSF1 | cleavage and polyadenylation specific factor 1 | 071894 | 173137 | 147804 | 18, 29, 26 |
| 8 | CLU | clusterin | 120885 | 120915 | 168077 | 40, 26, 10 |
| 8 | CCDC166 | coiled-coil domain containing 166 | 255181 | 181135 | 181085 | 38, |
| 8 | CCDC25 | coiled-coil domain containing 25 | 147419 | 168077 | 171320 | 29, 6, 44 |
| 8 | CHCHD7 | coiled-coil-helix-coiled-coil-helix domain containing 7 | 170791 | 181690 | 170786 | 41, 11, 7 |
| 8 | CTHRC1 | collagen triple helix repeat containing 1 | 164932 | 164930 | 164933 | 6, 35, 43 |
| 8 | COL14A1 | collagen type XIV alpha 1 chain | 187955 | 155792 | 172172 | 45, 11, 26 |
| 8 | COL22A1 | collagen type XXII alpha 1 chain | 169436 | 147724 | 169427 | 31, 29, 2 |
| 8 | COLEC10 | collectin subfamily member 10 | 184374 | 164761 | 147676 | 19, 35, 27 |
| 8 | COMMD5 | COMM domain containing 5 | 170619 | 147789 | 196150 | 22, 6, 26 |
| 8 | COPS5 | COP9 signalosome subunit 5 | 121022 | 178125 | 104218 | 41, 25, 22 |
| 8 | CPNE3 | copine 3 | 085719 | 176623 | 170289 | 24, 22, 42 |
| 8 | CRH | corticotropin releasing hormone | 147571 | 147573 | 179041 | 32, 28, 2 |
| 8 | CSMD1 | CUB and Sushi multiple domains 1 | 183117 | 036448 | 147316 | 38, 10, 8 |
| 8 | CSMD3 | CUB and Sushi multiple domains 3 | 164796 | 164794 | 104447 | 8, 38, 14 |
| 8 | CNBD1 | cyclic nucleotide binding domain containing 1 | 176571 | 170289 | 176566 | 38, 36, 37 |
| 8 | CNGB3 | cyclic nucleotide gated channel beta 3 | 170289 | 085719 | 176571 | 14, 38, 25 |
| 8 | CCNE2 | cyclin E2 | 175305 | 164941 | 156170 | 4, 22, 7 |
| 8 | CYHR1 | cysteine and histidine rich 1 | 187954 | 160949 | 167702 | 18, 33, 14 |
| 8 | CRISPLD1 | cysteine rich secretory protein LCCL domain containing 1 | 121005 | 137558 | 164749 | 11, 42, 24 |
| 8 | COX6C | cytochrome c oxidase subunit 6C | 164919 | 132549 | 132554 | 15, 25, 7 |
| 8 | CYC1 | cytochrome c1 | 179091 | 197858 | 179526 | 34, 25, 35 |
| 8 | CYP11B1 | cytochrome P450 family 11 subfamily B member 1 | 160882 | 104499 | 179142 | 2, 25, 23 |
| 8 | CYP11B2 | cytochrome P450 family 11 subfamily B member 2 | 179142 | 160882 | 160932 | 2, |
| 8 | CYP7A1 | cytochrome P450 family 7 subfamily A member 1 | 167910 | 215114 | 137575 | 19, 38, |
| 8 | CYP7B1 | cytochrome P450 family 7 subfamily B member 1 | 172817 | 180828 | 104442 | 23, 42, 33 |
| 8 | DCAF13 | DDB1 and CUL4 associated factor 13 | 164934 | 164933 | 176406 | 30, 43, 45 |
| 8 | DCAF4L2 | DDB1 and CUL4 associated factor 4 like 2 | 176566 | 176571 | 156103 | 38, 13, |
| 8 | DDHD2 | DDHD domain containing 2 | 085788 | 156735 | 147535 | 1, 26, 40 |
| 8 | DOCK5 | dedicator of cytokinesis 5 | 147459 | 277586 | 147437 | 11, 20, 9 |
| 8 | DEFA1 | defensin alpha 1 | 206047 | 164821 | 240247 | 7, 5, 19 |
| 8 | DEFA1B | defensin alpha 1B | 240247 | 206047 | 239839 | |
| 8 | DEFA3 | defensin alpha 3 | 239839 | 240247 | 164816 | 7, 5, 36 |
| 8 | DEFA4 | defensin alpha 4 | 164821 | 164822 | 206047 | 7, 5, 6 |
| 8 | DEFA5 | defensin alpha 5 | 164816 | 239839 | 230549 | 16, 37, 14 |
| 8 | DEFA6 | defensin alpha 6 | 164822 | 164825 | 164821 | 16, 37, |
| 8 | DEFB1 | defensin beta 1 | 164825 | 275591 | 164822 | 17, 10, 40 |
| 8 | DEFB103A | defensin beta 103A | 176797 | 178287 | 171711 | |
| 8 | DEFB103B | defensin beta 103B | 177243 | 177257 | 164871 | |
| 8 | DEFB104A | defensin beta 104A | 176782 | 186579 | 178287 | |
| 8 | DEFB104B | defensin beta 104B | 177023 | 164871 | 187082 | |
| 8 | DEFB105A | defensin beta 105A | 186562 | 186572 | 186579 | |
| 8 | DEFB105B | defensin beta 105B | 186599 | 187082 | 198129 | |
| 8 | DEFB106A | defensin beta 106A | 186579 | 186562 | 176782 | |
| 8 | DEFB106B | defensin beta 106B | 187082 | 177023 | 186599 | |
| 8 | DEFB107A | defensin beta 107A | 186572 | 255378 | 186562 | |
| 8 | DEFB107B | defensin beta 107B | 198129 | 186599 | 255251 | |
| 8 | DEFB130 | defensin beta 130 | 232948 | 186523 | 145002 | |
| 8 | DEFB134 | defensin beta 134 | 205882 | 205883 | 233050 | 38, |
| 8 | DEFB135 | defensin beta 135 | 205883 | 205884 | 205882 | |
| 8 | DEFB136 | defensin beta 136 | 205884 | 164733 | 205883 | |
| 8 | DEFB4A | defensin beta 4A | 171711 | 176797 | 215356 | |
| 8 | DEFB4B | defensin beta 4B | 177257 | 215372 | 177243 | |
| 8 | DMTN | dematin actin binding protein | 158856 | 158815 | 158863 | 26, 8, 19 |
| 8 | DCSTAMP | dendrocyte expressed seven transmembrane protein | 164935 | 147647 | 147650 | 40, 5, 20 |
| 8 | DENND3 | DENN domain containing 3 | 105339 | 281855 | 022567 | 22, 43, 7 |
| 8 | DEPTOR | DEP domain containing MTOR interacting protein | 155792 | 136982 | 187955 | 2, 1, 42 |
| 8 | DERL1 | derlin 1 | 136986 | 178764 | 156787 | 11, 1, 24 |
| 8 | DGAT1 | diacylglycerol O-acyltransferase 1 | 185000 | 185122 | 261678 | 26, 21, 37 |
| 8 | DKK4 | dickkopf WNT signaling pathway inhibitor 4 | 104371 | 070501 | 078668 | 12, 23, 37 |
| 8 | DPYS | dihydropyrimidinase | 147647 | 176406 | 164935 | 19, 17, 39 |
| 8 | DPYSL2 | dihydropyrimidinase like 2 | 092964 | 240694 | 120907 | 1, 8, 26 |
| 8 | DLC1 | DLC1 Rho GTPase activating protein | 164741 | 250305 | 164743 | 1, 45, 15 |
| 8 | DLGAP2 | DLG associated protein 2 | 198010 | 104714 | 283494 | |
| 8 | DLGAP2 | DLG associated protein 2 | 283494 | 198010 | 182372 | 24, 38, 20 |
| 8 | POLB | DNA polymerase beta | 070501 | 104365 | 104371 | 29, 27, 25 |
| 8 | DSCC1 | DNA replication and sister chromatid cohesion 1 | 136982 | 064313 | 155792 | 7, 3, 4 |
| 8 | DNAJC5B | DnaJ heat shock protein family (Hsp40) member C5 beta | 147570 | 205268 | 147573 | 38, 23, 32 |
| 8 | DOK2 | docking protein 2 | 147443 | 168546 | 130227 | 1, 5, 10 |
| 8 | DPY19L4 | dpy-19 like 4 (C. elegans) | 156162 | 104413 | 164941 | 11, 45, 1 |
| 8 | DUSP26 | dual specificity phosphatase 26 | 133878 | 133874 | 156687 | 25, 8, 14 |
| 8 | DUSP4 | dual specificity phosphatase 4 | 120875 | 197892 | 133872 | 3, 35, 23 |
| 8 | DCTN6 | dynactin subunit 6 | 104671 | 177669 | 157110 | 29, 4, 15 |
| 8 | E2F5 | E2F transcription factor 5 | 133740 | 133739 | 176731 | 22, 29, 27 |
| 8 | EBF2 | early B-cell factor 2 | 221818 | 184661 | 221914 | 42, 37, 18 |
| 8 | EGR3 | early growth response 3 | 179388 | 147439 | 134020 | 11, 26, 31 |
| 8 | ENTPD4 | ectonucleoside triphosphate diphosphohydrolase 4 | 197217 | 134013 | 147454 | 22, 7, 12 |
| 8 | ENPP2 | ectonucleotide pyrophosphatase/phosphodiesterase 2 | 136960 | 136999 | 064313 | 26, 8, 11 |
| 8 | EFCAB1 | EF-hand calcium binding domain 1 | 034239 | 169139 | 019549 | 31, 42, 20 |
| 8 | EFR3A | EFR3 homolog A | 132294 | 155897 | 253117 | 6, 31, 13 |
| 8 | ELP3 | elongator acetyltransferase complex subunit 3 | 134014 | 189233 | 168081 | 18, 2, 39 |
| 8 | ENY2 | ENY2, transcription and export complex 2 subunit | 120533 | 120526 | 205038 | 41, 34, 29 |
| 8 | EPPK1 | epiplakin 1 | 261150 | 185189 | 178209 | 18, 23, 24 |
| 8 | ESRP1 | epithelial splicing regulatory protein 1 | 104413 | 164944 | 156162 | 4, 3, 13 |
| 8 | EPHX2 | epoxide hydrolase 2 | 120915 | 120903 | 120885 | 39, 17, 2 |
| 8 | ERLIN2 | ER lipid raft associated 2 | 147475 | 183779 | 147471 | 13, 12, 6 |
| 8 | EMC2 | ER membrane protein complex subunit 2 | 104412 | 104408 | 164841 | 31, 1, 4 |
| 8 | ESCO2 | establishment of sister chromatid cohesion N-acetyltransferase 2 | 171320 | 147419 | 168078 | 22, 4, 11 |
| 8 | EBAG9 | estrogen receptor binding site associated, antigen, 9 | 147654 | 205038 | 147642 | 31, 34, 43 |
| 8 | EEF1D | eukaryotic translation elongation factor 1 delta | 104529 | 147813 | 179886 | |
| 8 | EIF3E | eukaryotic translation initiation factor 3 subunit E | 104408 | 147655 | 104412 | |
| 8 | EIF3H | eukaryotic translation initiation factor 3 subunit H | 147677 | 104447 | 147679 | 41, 1, 21 |
| 8 | EIF4EBP1 | eukaryotic translation initiation factor 4E binding protein 1 | 187840 | 188778 | 129691 | 3, 34, 43 |
| 8 | ERI1 | exoribonuclease 1 | 104626 | 147324 | 173281 | 22, 7, 6 |
| 8 | EXOSC4 | exosome component 4 | 178896 | 178814 | 197858 | 25, 26, 33 |
| 8 | EXT1 | exostosin glycosyltransferase 1 | 182197 | 164758 | 177570 | 18, 34, 13 |
| 8 | EXTL3 | exostosin like glycosyltransferase 3 | 012232 | 104290 | 104299 | 3, 1, 14 |
| 8 | XPO7 | exportin 7 | 130227 | 147443 | 158806 | 29, 31, 37 |
| 8 | EYA1 | EYA transcriptional coactivator and phosphatase 1 | 104313 | 221947 | 178860 | 11, 42, 26 |
| 8 | FAM83H-AS1 | FAM83H antisense RNA 1 | 203499 | 180921 | 180900 | |
| 8 | FAM110B | family with sequence similarity 110 member B | 169122 | 104331 | 215114 | 11, 17, 29 |
| 8 | FAM135B | family with sequence similarity 135 member B | 147724 | 131773 | 169436 | |
| 8 | FAM150A | family with sequence similarity 150 member A | 196711 | 147488 | 023287 | 44, 16, |
| 8 | FAM160B2 | family with sequence similarity 160 member B2 | 158863 | 158856 | 275074 | 32, 39, 36 |
| 8 | FAM167A | family with sequence similarity 167 member A | 154319 | 177710 | 136573 | 34, 1, 26 |
| 8 | FAM49B | family with sequence similarity 49 member B | 153310 | 147697 | 153317 | 7, 34, 4 |
| 8 | FAM83A | family with sequence similarity 83 member A | 147689 | 156787 | 189376 | 24, 18, 9 |
| 8 | FAM83H | family with sequence similarity 83 member H | 180921 | 181085 | 203499 | |
| 8 | FAM84B | family with sequence similarity 84 member B | 168672 | 173334 | 212993 | 3, 4, 23 |
| 8 | FAM86B1 | family with sequence similarity 86 member B1 | 186523 | 223443 | 232948 | 26, 3, 9 |
| 8 | FAM86B2 | family with sequence similarity 86 member B2 | 145002 | 232948 | 154359 | 18, 9, 45 |
| 8 | FAM91A1 | family with sequence similarity 91 member A1 | 176853 | 104537 | 214814 | 4, 2, 6 |
| 8 | FAM92A | family with sequence similarity 92 member A | 188343 | 205133 | 183808 | |
| 8 | FDFT1 | farnesyl-diphosphate farnesyltransferase 1 | 079459 | 154328 | 164733 | |
| 8 | FNTA | farnesyltransferase, CAAX box, alpha | 168522 | 254673 | 185900 | 11, 29, 26 |
| 8 | FABP12 | fatty acid binding protein 12 | 197416 | 170323 | 133731 | 38, 27, 14 |
| 8 | FABP4 | fatty acid binding protein 4 | 170323 | 205186 | 197416 | 1, 15, 4 |
| 8 | FABP5 | fatty acid binding protein 5 | 164687 | 076641 | 147588 | 44, 6, 30 |
| 8 | FABP9 | fatty acid binding protein 9 | 205186 | 147588 | 170323 | |
| 8 | FBXL6 | F-box and leucine rich repeat protein 6 | 182325 | 185803 | 173137 | 37, 22, 27 |
| 8 | FBXO16 | F-box protein 16 | 214050 | 186918 | 104290 | 31, 29, 28 |
| 8 | FBXO25 | F-box protein 25 | 147364 | 172748 | 180190 | 33, 38, 3 |
| 8 | FBXO32 | F-box protein 32 | 156804 | 156795 | 175946 | 25, 18, 34 |
| 8 | FBXO43 | F-box protein 43 | 156509 | 132554 | 147669 | 26, 5, 19 |
| 8 | FER1L6 | fer-1 like family member 6 | 214814 | 176853 | 164983 | 30, 38, |
| 8 | FGL1 | fibrinogen like 1 | 104760 | 129422 | 078674 | 19, 4, 25 |
| 8 | FSBP | fibrinogen silencer binding protein | 265817 | 283460 | 164944 | 7, 44, 39 |
| 8 | FGF17 | fibroblast growth factor 17 | 158815 | 158806 | 158856 | 12, 14, 8 |
| 8 | FGF20 | fibroblast growth factor 20 | 078579 | 038945 | 155970 | 16, 14, 20 |
| 8 | FGFR1 | fibroblast growth factor receptor 1 | 077782 | 165046 | 196166 | 42, |
| 8 | FOXH1 | forkhead box H1 | 160973 | 167702 | 160972 | 12, 27, 38 |
| 8 | FZD3 | frizzled class receptor 3 | 104290 | 214050 | 012232 | 11, 14, 22 |
| 8 | FZD6 | frizzled class receptor 6 | 164930 | 164929 | 164932 | 42, 40, 18 |
| 8 | FUT10 | fucosyltransferase 10 | 172728 | 157168 | 129696 | 26, 27, 23 |
| 8 | GPR20 | G protein-coupled receptor 20 | 204882 | 022567 | 184489 | 26, 36, 10 |
| 8 | GGH | gamma-glutamyl hydrolase | 137563 | 185942 | 137561 | 6, 19, 17 |
| 8 | GDAP1 | ganglioside induced differentiation associated protein 1 | 104381 | 104369 | 137558 | 26, 18, 8 |
| 8 | GSDMC | gasdermin C | 147697 | 280151 | 153310 | 36, 24, 42 |
| 8 | GSDMD | gasdermin D | 104518 | 014164 | 204839 | 39, 34, 36 |
| 8 | GATA4 | GATA binding protein 4 | 136574 | 136573 | 154328 | 27, 38, 37 |
| 8 | GFRA2 | GDNF family receptor alpha 2 | 168546 | 061337 | 147443 | 14, 32, 6 |
| 8 | GTF2E2 | general transcription factor IIE subunit 2 | 197265 | 157110 | 253457 | 29, 11, 21 |
| 8 | GINS4 | GINS complex subunit 4 | 147536 | 147533 | 158669 | 9, 5, 19 |
| 8 | GLI4 | GLI family zinc finger 4 | 250571 | 264668 | 185730 | 33, 17, 34 |
| 8 | GRINA | glutamate ionotropic receptor NMDA type subunit associated protein 1 | 178719 | 178685 | 186583 | 27, 33, 28 |
| 8 | ERICH1 | glutamate rich 1 | 104714 | 180190 | 198010 | 12, |
| 8 | ERICH5 | glutamate rich 5 | 177459 | 156482 | 132541 | |
| 8 | GOT1L1 | glutamic-oxaloacetic transaminase 1-like 1 | 169154 | 156675 | 188778 | 38, |
| 8 | GPT | glutamic--pyruvic transaminase | 167701 | 160972 | 167700 | 1, 15, 16 |
| 8 | GSR | glutathione-disulfide reductase | 104687 | 253457 | 104691 | 29, 1, 37 |
| 8 | GPAT4 | glycerol-3-phosphate acyltransferase 4 | 158669 | 147536 | 165066 | 35, 27, 41 |
| 8 | GPAA1 | glycosylphosphatidylinositol anchor attachment 1 | 197858 | 178896 | 179091 | 36, 35, 9 |
| 8 | GPIHBP1 | glycosylphosphatidylinositol anchored high density lipoprotein binding protein 1 | 277494 | 176956 | 181638 | 15, 20, 10 |
| 8 | GML | glycosylphosphatidylinositol anchored molecule like | 104499 | 167656 | 160882 | 38, |
| 8 | GOLGA7 | golgin A7 | 147533 | 104332 | 147536 | 43, 7, 8 |
| 8 | GNRH1 | gonadotropin releasing hormone 1 | 147437 | 147459 | 104756 | 25, 20, 44 |
| 8 | GRHL2 | grainyhead like transcription factor 2 | 083307 | 120963 | 104490 | 18, 42, 23 |
| 8 | GDF6 | growth differentiation factor 6 | 156466 | 156172 | 156467 | 6, 12, 38 |
| 8 | GEM | GTP binding protein overexpressed in skeletal muscle | 164949 | 079112 | 197275 | 37, 45, 43 |
| 8 | HSF1 | heat shock transcription factor 1 | 185122 | 260428 | 185000 | 2, 39, 35 |
| 8 | HGSNAT | heparan-alpha-glucosaminide N-acetyltransferase | 165102 | 185900 | 164808 | 29, 40, 30 |
| 8 | HNF4G | hepatocyte nuclear factor 4 gamma | 164749 | 121005 | 091656 | |
| 8 | HHLA1 | HERV-H LTR-associating 1 | 132297 | 258417 | 184156 | 38, |
| 8 | HEY1 | hes related family bHLH transcription factor with YRPW motif 1 | 164683 | 104435 | 147586 | 11, 42, 26 |
| 8 | HGH1 | HGH1 homolog | 235173 | 179698 | 179832 | 3, 21, 27 |
| 8 | HMBOX1 | homeobox containing 1 | 147421 | 104299 | 197892 | 2, 42, 34 |
| 8 | HOOK3 | hook microtubule tethering protein 3 | 168172 | 120925 | 254673 | |
| 8 | HR | HR, lysine demethylase and nuclear receptor corepressor | 168453 | 275074 | 168476 | 18, 35, 8 |
| 8 | HTRA4 | HtrA serine peptidase 4 | 169495 | 169499 | 169490 | 32, 40, 44 |
| 8 | HAS2 | hyaluronan synthase 2 | 170961 | 172164 | 178764 | 45, 12, 7 |
| 8 | IDO1 | indoleamine 2,3-dioxygenase 1 | 131203 | 104755 | 188676 | 30, 32, 44 |
| 8 | IDO2 | indoleamine 2,3-dioxygenase 2 | 188676 | 131203 | 176907 | 32, 19, 44 |
| 8 | IKBKB | inhibitor of nuclear factor kappa B kinase subunit beta | 104365 | 104368 | 070501 | 11, 1, 39 |
| 8 | IMPA1 | inositol monophosphatase 1 | 133731 | 197416 | 253598 | 7, 4, 10 |
| 8 | IMPAD1 | inositol monophosphatase domain containing 1 | 104331 | 181195 | 169122 | 11, 1, 40 |
| 8 | INTS10 | integrator complex subunit 10 | 104613 | 147408 | 175445 | 3, 43, 33 |
| 8 | INTS8 | integrator complex subunit 8 | 164941 | 156162 | 175305 | 11, 24, 22 |
| 8 | INTS9 | integrator complex subunit 9 | 104299 | 012232 | 147421 | 21, 41, 9 |
| 8 | IL7 | interleukin 7 | 104432 | 104427 | 104435 | 31, 22, 24 |
| 8 | JRK | Jrk helix-turn-helix protein | 234616 | 198576 | 167653 | 18, 1, 23 |
| 8 | JPH1 | junctophilin 1 | 104369 | 154589 | 104381 | 6, 25, 24 |
| 8 | KLHL38 | kelch like family member 38 | 175946 | 156804 | 104537 | 15, |
| 8 | KBTBD11 | kelch repeat and BTB domain containing 11 | 176595 | 104728 | 036448 | 8, 17, 14 |
| 8 | KHDRBS3 | KH RNA binding domain containing, signal transduction associated 3 | 131773 | 066827 | 147724 | 10, 34, 38 |
| 8 | KIAA0196 | KIAA0196 | 164961 | 104549 | 156831 | 42, 22, 11 |
| 8 | KIAA1429 | KIAA1429 | 164944 | 265817 | 104413 | 19, 43, 31 |
| 8 | KIAA1456 | KIAA1456 | 250305 | 154359 | 164741 | 40, 28, 20 |
| 8 | KIF13B | kinesin family member 13B | 197892 | 147421 | 120875 | 29, 26, 16 |
| 8 | KIFC2 | kinesin family member C2 | 167702 | 187954 | 160973 | 1, 26, 3 |
| 8 | KLF10 | Kruppel like factor 10 | 155090 | 155087 | 155096 | 1, 24, 22 |
| 8 | LACTB2 | lactamase beta 2 | 147592 | 067167 | 221947 | 34, 4, 24 |
| 8 | LRP12 | LDL receptor related protein 12 | 147650 | 164935 | 169946 | 18, 26, 11 |
| 8 | LEPROTL1 | leptin receptor overlapping transcript-like 1 | 104660 | 133872 | 177669 | 38, 7, 32 |
| 8 | LRRCC1 | leucine rich repeat and coiled-coil centrosomal protein 1 | 133739 | 184672 | 133740 | 26, 38, 12 |
| 8 | LRRC14 | leucine rich repeat containing 14 | 160959 | 160957 | 254402 | 3, 35, 27 |
| 8 | LRRC24 | leucine rich repeat containing 24 | 254402 | 160959 | 213563 | 14, 24, 6 |
| 8 | LRRC6 | leucine rich repeat containing 6 | 129295 | 184156 | 165071 | 10, 29, 38 |
| 8 | LRRC69 | leucine rich repeat containing 69 | 214954 | 155100 | 147606 | |
| 8 | LGI3 | leucine rich repeat LGI family member 3 | 168481 | 168476 | 168484 | 26, 35, 4 |
| 8 | LETM2 | leucine zipper and EF-hand containing transmembrane protein 2 | 165046 | 147548 | 077782 | |
| 8 | LZTS1 | leucine zipper tumor suppressor 1 | 061337 | 147416 | 168546 | 26, 31, 35 |
| 8 | LPL | lipoprotein lipase | 175445 | 104613 | 036565 | 1, 29, 15 |
| 8 | LONRF1 | LON peptidase N-terminal domain and ring finger 1 | 154359 | 145002 | 250305 | 26, 22, 33 |
| 8 | LSM1 | LSM1 homolog, mRNA degradation associated | 175324 | 147465 | 156735 | 37, 3, 11 |
| 8 | LYNX1 | Ly6/neurotoxin 1 | 180155 | 197353 | 167656 | 29, 1, 8 |
| 8 | LYPD2 | LY6/PLAUR domain containing 2 | 197353 | 126233 | 180155 | 24, 14, 20 |
| 8 | LY6D | lymphocyte antigen 6 complex, locus D | 167656 | 180155 | 104499 | 13, 18, 9 |
| 8 | LY6E | lymphocyte antigen 6 complex, locus E | 160932 | 179142 | 261667 | 27, 34, 35 |
| 8 | LY6H | lymphocyte antigen 6 complex, locus H | 176956 | 261667 | 277494 | 8, 27, 10 |
| 8 | LY6K | lymphocyte antigen 6 complex, locus K | 160886 | 167653 | 130193 | 9, 35, 6 |
| 8 | LY6L | lymphocyte antigen 6 complex, locus L | 261667 | 160932 | 176956 | |
| 8 | LY96 | lymphocyte antigen 96 | 154589 | 175606 | 104369 | 2, 39, 22 |
| 8 | LYN | LYN proto-oncogene, Src family tyrosine kinase | 254087 | 137574 | 008988 | 41, 21, 22 |
| 8 | KAT6A | lysine acetyltransferase 6A | 083168 | 029534 | 070718 | 18, 40, 30 |
| 8 | LYPLA1 | lysophospholipase I | 120992 | 280473 | 137547 | 7, 38, 4 |
| 8 | LAPTM4B | lysosomal protein transmembrane 4 beta | 104341 | 147649 | 132561 | 29, 15, 38 |
| 8 | LOXL2 | lysyl oxidase like 2 | 134013 | 104679 | 197217 | |
| 8 | MSR1 | macrophage scavenger receptor 1 | 038945 | 104723 | 078579 | 25, 8, |
| 8 | MROH1 | maestro heat like repeat family member 1 | 179832 | 235173 | 261236 | 40, 18, 6 |
| 8 | MROH5 | maestro heat like repeat family member 5 | 226807 | 184489 | 226490 | 38, |
| 8 | MROH6 | maestro heat like repeat family member 6 | 204839 | 104518 | 147813 | |
| 8 | MAFA | MAF bZIP transcription factor A | 182759 | 158106 | 014164 | |
| 8 | MAF1 | MAF1 homolog, negative regulator of RNA polymerase III | 179632 | 179526 | 179698 | 41, 9, 27 |
| 8 | MFSD3 | major facilitator superfamily domain containing 3 | 167700 | 167701 | 160957 | 9, 7, 31 |
| 8 | MAK16 | MAK16 homolog | 198042 | 129696 | 133874 | 13, 35, 18 |
| 8 | MAL2 | mal, T-cell differentiation protein 2 (gene/pseudogene) | 147676 | 184374 | 136999 | 4, 16, 29 |
| 8 | MFHAS1 | malignant fibrous histiocytoma amplified sequence 1 | 147324 | 253958 | 104626 | 22, 24, 41 |
| 8 | MATN2 | matrilin 2 | 132561 | 104341 | 156482 | 44, 26, 42 |
| 8 | MMP16 | matrix metallopeptidase 16 | 156103 | 176566 | 104312 | 45, 15, 24 |
| 8 | MTBP | MDM2 binding protein | 172167 | 172172 | 172164 | 18, 13, 24 |
| 8 | MED30 | mediator complex subunit 30 | 164758 | 164756 | 182197 | 22, 27, 21 |
| 8 | MBOAT4 | membrane bound O-acyltransferase domain containing 4 | 177669 | 104660 | 104671 | |
| 8 | MTDH | metadherin | 147649 | 180543 | 104341 | 22, 11, 29 |
| 8 | MSRA | methionine sulfoxide reductase A | 175806 | 173273 | 253649 | 11, 17, 10 |
| 8 | MCPH1 | microcephalin 1 | 147316 | 183117 | 091879 | |
| 8 | MTUS1 | microtubule associated tumor suppressor 1 | 129422 | 104213 | 104760 | |
| 8 | MCM4 | minichromosome maintenance complex component 4 | 104738 | 253729 | 169139 | 24, 9, 22 |
| 8 | MCMDC2 | minichromosome maintenance domain containing 2 | 178460 | 104205 | 261787 | 29, 38, 42 |
| 8 | MICU3 | mitochondrial calcium uptake family member 3 | 155970 | 078579 | 104219 | 28, 8, 32 |
| 8 | MTFR1 | mitochondrial fission regulator 1 | 066855 | 104442 | 205268 | |
| 8 | MRPL13 | mitochondrial ribosomal protein L13 | 172172 | 187955 | 172167 | 29, 10, 43 |
| 8 | MRPL15 | mitochondrial ribosomal protein L15 | 137547 | 120992 | 164736 | 29, 22, 15 |
| 8 | MRPS28 | mitochondrial ribosomal protein S28 | 147586 | 164683 | 276418 | 29, 10, 15 |
| 8 | MTERF3 | mitochondrial transcription termination factor 3 | 156469 | 156467 | 156471 | 18, 2, 45 |
| 8 | MAPK15 | mitogen-activated protein kinase 15 | 181085 | 255181 | 180921 | 13, 23, 18 |
| 8 | MOS | MOS proto-oncogene, serine/threonine kinase | 172680 | 008988 | 181690 | 38, |
| 8 | MTSS1 | MTSS1, l-BAR domain containing | 170873 | 147684 | 180938 | 24, 18, 26 |
| 8 | MSC | musculin | 178860 | 104313 | 104321 | 35, 32, 43 |
| 8 | MYBL1 | MYB proto-oncogene like 1 | 185697 | 169085 | 175073 | 22, 21, 45 |
| 8 | MYOM2 | myomesin 2 | 036448 | 176595 | 183117 | 15, 25, 26 |
| 8 | MTMR7 | myotubularin related protein 7 | 003987 | 155975 | 003989 | 31, 28, 8 |
| 8 | MTMR9 | myotubularin related protein 9 | 104643 | 280273 | 177710 | 11, 34, 27 |
| 8 | NAT1 | N-acetyltransferase 1 | 171428 | 104763 | 156006 | 31, 29, 28 |
| 8 | NAT2 | N-acetyltransferase 2 | 156006 | 171428 | 156011 | 19, 16, 6 |
| 8 | ASAH1 | N-acylsphingosine amidohydrolase 1 | 104763 | 078674 | 171428 | |
| 8 | NDUFAF6 | NADH: ubiquinone oxidoreductase complex assembly factor 6 | 156170 | 175305 | 164938 | 10, |
| 8 | NDUFB9 | NADH: ubiquinone oxidoreductase subunit B9 | 147684 | 147687 | 170873 | 43, 41, 35 |
| 8 | NEIL2 | nei like DNA glycosylase 2 | 154328 | 136574 | 079459 | 9, 6, 45 |
| 8 | NOV | nephroblastoma overexpressed | 136999 | 147676 | 136960 | 2, 11, 45 |
| 8 | NRG1 | neuregulin 1 | 157168 | 165392 | 172728 | 10, 17, 45 |
| 8 | NCALD | neurocalcin delta | 104490 | 083307 | 048392 | 42, 8, 34 |
| 8 | NEFL | neurofilament light | 277586 | 104722 | 147459 | 26, 11, 8 |
| 8 | NEFM | neurofilament medium | 104722 | 069206 | 277586 | |
| 8 | NPBWR1 | neuropeptides B and W receptor 1 | 183729 | 023287 | 082556 | 20, |
| 8 | NSMAF | neutral sphingomyelinase activation asscciated factor | 035681 | 137575 | 198846 | 1, 9, 4 |
| 8 | NBN | nibrin | 104320 | 164823 | 104325 | 4, 10, 40 |
| 8 | NAPRT | nicotinate phosphoribosyltransferase | 147813 | 204839 | 104529 | 38, 34, 37 |
| 8 | NIPAL2 | NIPA like domain containing 2 | 104361 | 104356 | 104375 | 42, 24, 15 |
| 8 | NKX2-6 | NK2 homeobox 6 | 180053 | 167034 | 159167 | 12, |
| 8 | NKX3-1 | NK3 homeobox 1 | 167034 | 147454 | 180053 | 33, 38, 6 |
| 8 | NKX6-3 | NK6 homeobox 3 | 165066 | 158669 | 029534 | |
| 8 | NDRG1 | N-myc downstream regulated 1 | 104419 | 104415 | 008513 | 24, 39, 33 |
| 8 | NSMCE2 | NSE2/MMS21 homolog, SMC5-SMC6 complex SUMO ligase | 156831 | 164961 | 173334 | 36, 10, 26 |
| 8 | NECAB1 | N-terminal EF-hand calcium binding protein 1 | 123119 | 180694 | 253250 | 8, 17, 38 |
| 8 | NUGGC | nuclear GTPase, germinal center associated | 189233 | 168079 | 134014 | 22, 21, 19 |
| 8 | NRBP2 | nuclear receptor binding protein 2 | 185189 | 179950 | 261150 | 27, 6, 40 |
| 8 | NCOA2 | nuclear receptor coactivator 2 | 140396 | 147596 | 067167 | 1, 26, 22 |
| 8 | NPM2 | nucleophosmin/nudeoplasmin 2 | 158806 | 130227 | 158815 | 8, 35, 28 |
| 8 | NUDCD1 | NudC domain containing 1 | 120526 | 174417 | 120533 | 42, 1, 3 |
| 8 | NUDT18 | nudix hydrolase 18 | 275074 | 158863 | 168453 | 34, 27, 5 |
| 8 | OSR2 | odd-skipped related transciption factor 2 | 164920 | 156486 | 132549 | 44, 38, 27 |
| 8 | OR4F21 | olfactory receptor family 4 subfamily F member 21 | 176269 | 164935 | 172748 | |
| 8 | OPRK1 | opioid receptor kappa 1 | 082556 | 183729 | 047249 | 32, 33, 38 |
| 8 | OC90 | otoconin 90 | 253117 | 132294 | 258417 | 12, |
| 8 | OTUD6B | OTU domain containing 6B | 155100 | 155099 | 214954 | 22, 42, 43 |
| 8 | ODF1 | outer dense fiber of sperm tails 1 | 155087 | 104517 | 155090 | 38, 10, |
| 8 | OXR1 | oxidation resistance 1 | 164830 | 169946 | 174429 | 42, 2, 8 |
| 8 | OSGIN2 | oxidative stress induced growth inhibitor family member 2 | 164823 | 104312 | 104320 | 29, 7, 1 |
| 8 | PNMA2 | paraneoplastic Ma antigen 2 | 240694 | 104765 | 092964 | 8, 45, 26 |
| 8 | PDLIM2 | PDZ and LIM domain 2 | 120913 | 120896 | 248235 | 36, 22, 32 |
| 8 | PBK | PDZ binding kinase | 168078 | 171320 | 168079 | 38, 3, 19 |
| 8 | PI15 | peptidase inhibitor 15 | 137558 | 104381 | 121005 | 11, 30, 37 |
| 8 | PCM1 | pericentriolar material 1 | 078674 | 104760 | 104763 | 42, 38, 37 |
| 8 | PMP2 | peripheral myelin protein 2 | 147588 | 164687 | 205186 | 26, 8, 11 |
| 8 | PXDNL | peroxidasin like | 147485 | 147481 | 168300 | 15, 40, 38 |
| 8 | PEX2 | peroxisomal biogenesis factor 2 | 164751 | 091656 | 171033 | 7, 4, 13 |
| 8 | PHF20L1 | PHD finger protein 20-like 1 | 129292 | 165071 | 042832 | 29, 22, 37 |
| 8 | PEBP4 | phosphatidylethanolamine binding protein 4 | 134020 | 179388 | 008853 | 29, 25, 20 |
| 8 | PREX2 | phosphatidylinositol-3,4,5-trisphosphate dependent Rac exchange factor 2 | 046889 | 165078 | 165084 | 40, 45, 23 |
| 8 | PTDSS1 | phosphatidylserine synthase 1 | 156471 | 156469 | 169439 | 21, 7, 5 |
| 8 | PDE7A | phosphodiesterase 7A | 205268 | 066855 | 147570 | 22, 39, 26 |
| 8 | PLPP5 | phospholipid phosphatase 5 | 147535 | 085788 | 147548 | 33, 37, 20 |
| 8 | PAG1 | phosphoprotein membrane anchor with glycosphingolipid microdomains 1 | 076641 | 164684 | 164687 | 22, 39, 7 |
| 8 | PHYHIP | phytanoyl-CoA 2-hydroxylase interacting protein | 168490 | 168487 | 168495 | 8, 34, 14 |
| 8 | PINX1 | PIN2/TERF1 interacting, telomerase inhibitor 1 | 254093 | 258724 | 171044 | |
| 8 | PIWIL2 | piwi like RNA-mediated gene silencing 2 | 197181 | 168495 | 104635 | 38, 25, 16 |
| 8 | PLAG1 | PLAG1 zinc finger | 181690 | 172680 | 170791 | 31, 11, 30 |
| 8 | PLAT | plasminogen activator, tissue type | 104368 | 070718 | 104365 | 45, 29, 35 |
| 8 | PDGFRL | platelet derived growth factor receptor like | 104213 | 003989 | 129422 | 11, 1, 6 |
| 8 | PSD3 | pleckstrin and Sec7 domain containing 3 | 156011 | 156006 | 104611 | 29, 8, 26 |
| 8 | PLEKHF2 | pleckstrin homology and FYVE domain containing 2 | 175895 | 164938 | 156172 | 22, 41, 23 |
| 8 | PLEKHA2 | pleckstrin homology domain containing A2 | 169499 | 147526 | 169495 | 22, 7, 29 |
| 8 | PLEC | plectin | 178209 | 261150 | 178685 | 26, 18, 28 |
| 8 | PABPC1 | poly(A) binding protein cytoplasmic 1 | 070756 | 174226 | 164924 | 18, 11, 37 |
| 8 | PARP10 | poly(ADP-ribose) polymerase family member 10 | 178685 | 178209 | 178719 | 39, 42, 36 |
| 8 | PUF60 | poly(U) binding splicing factor 60 | 179950 | 180900 | 185189 | 3, 41, 27 |
| 8 | PKHD1L1 | polycystic kidney and hepatic disease 1 (autosomal recessive)-like 1 | 205038 | 120533 | 147654 | 1, 2, 44 |
| 8 | POP1 | POP1 homolog, ribonuclease P/MRP subunit | 104356 | 132541 | 104361 | 13, 9, 1 |
| 8 | KCNU1 | potassium calcium-activated channel subfamily U member 1 | 215262 | 156687 | 183779 | 38, 10, 39 |
| 8 | KCTD9 | potassium channel tetramerization domain containing 9 | 104756 | 147437 | 184661 | 26, 34, 19 |
| 8 | KCNK9 | potassium two pore domain channel subfamily K member 9 | 169427 | 169436 | 167632 | 8, 33, |
| 8 | KCNS2 | potassium voltage-gated channel modifier subfamily S member 2 | 156486 | 104375 | 164920 | 32, 44, 14 |
| 8 | KCNV1 | potassium voltage-gated channel modifier subfamily V member 1 | 164794 | 147642 | 164796 | 8, 17, 12 |
| 8 | KCNB2 | potassium voltage-gated channel subfamily B member 2 | 182674 | 104321 | 147601 | 38, 8, |
| 8 | KCNQ3 | potassium voltage-gated channel subfamily Q member 3 | 184156 | 132297 | 129295 | 8, 25, 2 |
| 8 | POU5F1B | POU class 5 homeobox 1B | 212993 | 168672 | 279524 | 3, 16, 42 |
| 8 | PRDM14 | PR/SET domain 14 | 147596 | 137571 | 140396 | 12, 30, 38 |
| 8 | PNOC | prepronociceptin | 168081 | 134014 | 186918 | 41, 22, 36 |
| 8 | PENK | proenkephalin | 181195 | 170786 | 104331 | |
| 8 | PRR23D1 | proline rich 23 domain containing 1 | 255251 | 198129 | 255378 | |
| 8 | PRR23D2 | proline rich 23 domain containing 2 | 255378 | 255251 | 186572 | |
| 8 | PROSC | proline synthetase cotranscribed homolog (bacterial) | 147471 | 147475 | 020181 | 29, 43, 16 |
| 8 | PSCA | prostate stem cell antigen | 167653 | 234616 | 160886 | 37, 33, 35 |
| 8 | PRSS51 | Protease, serine 51 | 253649 | 175806 | 184647 | 40, 25, 8 |
| 8 | PRSS55 | protease, serine 55 | 184647 | 253649 | 183638 | 38, 8, |
| 8 | PRKDC | protein kinase, DNA-activated, catalytic polypeptide | 253729 | 221869 | 104738 | 30, 4, 26 |
| 8 | PPP1R16A | protein phosphatase 1 regulatory subunit 16A | 160972 | 160973 | 167701 | 3, 31, 17 |
| 8 | PPP1R3B | protein phosphatase 1 regulatory subunit 3B | 173281 | 104626 | 173273 | 29, 25, 26 |
| 8 | PPP1R42 | protein phosphatase 1 regulatory subunit 42 | 178125 | 261787 | 121022 | |
| 8 | PPP2CB | protein phosphatase 2 catalytic subunit beta | 104695 | 104691 | 133863 | 11, 7, 4 |
| 8 | PPP2R2A | protein phosphatase 2 regulatory subunit Balpha | 221914 | 221818 | 104765 | 4, 1, 2 |
| 8 | PPP3CC | protein phosphatase 3 catalytic subunit gamma | 120910 | 104635 | 120896 | 22, 38, 29 |
| 8 | PSKH2 | protein serine kinase H2 | 147613 | 147614 | 164893 | |
| 8 | PTK2 | protein tyrosine kinase 2 | 169398 | 123908 | 281855 | |
| 8 | PTK2B | protein tyrosine kinase 2 beta | 120899 | 104228 | 120903 | 37, 41, 21 |
| 8 | PTP4A3 | protein tyrosine phosphatase type IVA, member 3 | 184489 | 204882 | 226807 | 26, 25, 27 |
| 8 | PCMTD1 | protein-L-isoaspartate (D-aspartate) O-methyltransferase domain containing 1 | 168300 | 147485 | 147488 | 27, 42, 1 |
| 8 | POMK | protein-O-mannose kinase | 185900 | 168522 | 165102 | |
| 8 | PURG | purine rich element binding protein G | 172733 | 133863 | 165392 | 33, 8, 27 |
| 8 | PYCRL | pyrroline-5-carboxylate reductase-like | 104524 | 179886 | 104522 | 34, 13, 9 |
| 8 | PDP1 | pyruvate dehyrogenase phosphatase catalytic subunit 1 | 164951 | 164953 | 079112 | 2, 25, 8 |
| 8 | R3HCC1 | R3H domain and coiled-coil containing 1 | 104679 | 147457 | 134013 | 1, 34, 5 |
| 8 | RAB11FIP1 | RAB11 family interacting protein 1 | 156675 | 104221 | 169154 | 3, 22, 16 |
| 8 | RAB2A | RAB2A, member RAS oncogene family | 104388 | 178538 | 171316 | 29, 31, 15 |
| 8 | RAD21 | RAD21 cohesin complex component | 164754 | 147679 | 281527 | 34, 22, 39 |
| 8 | RAD54B | RAD54 homolog B (S. cerevisiae) | 197275 | 164949 | 283460 | 11, 23, 3 |
| 8 | RAD54B | RAD54 homolog B (S. cerevisiae) | 283460 | 197275 | 265817 | 11, 23, 3 |
| 8 | RALYL | RALY RNA binding protein-like | 184672 | 104497 | 133739 | |
| 8 | RB1CC1 | RB1 inducible coiled-coil 1 | 023287 | 196711 | 183729 | 24, 23, 7 |
| 8 | RIDA | reactive intermediate imine deaminase A homolog | 132541 | 177459 | 104356 | |
| 8 | REEP4 | receptor accessory protein 4 | 168476 | 168453 | 168481 | 9, 39, 21 |
| 8 | RIPK2 | receptor interacting serine/threonine kinase 2 | 104312 | 156103 | 164823 | 45, 9, 37 |
| 8 | RECQL4 | RecQ like helicase 4 | 160957 | 167700 | 160959 | 41, 21, 3 |
| 8 | RIMS2 | regulating synaptic membrane exocytosis 2 | 176406 | 164934 | 147647 | 31, 2, 8 |
| 8 | RGS20 | regulator of G-protein signaling 20 | 147509 | 047249 | 187735 | 11, 6, 35 |
| 8 | RGS22 | regulator of G-protein signaling 22 | 132554 | 164919 | 156509 | 38, 30, 23 |
| 8 | RMDN1 | regulator of microtubule dynamics 1 | 176623 | 123124 | 085719 | |
| 8 | RDH10 | retinol dehydrogenase 10 | 121039 | 147604 | 040341 | 44, 14, 17 |
| 8 | ARHGAP39 | Rho GTPase activating protein 39 | 147799 | 213563 | 198169 | 3, 9, 42 |
| 8 | ARHGEF10 | Rho guanine nucleotide exchange factor 10 | 104728 | 283239 | 176595 | 39, 6, 12 |
| 8 | RHOBTB2 | Rho related BTB domain containing 2 | 008853 | 134020 | 120889 | 21, 4, 22 |
| 8 | RHPN1 | rhophilin Rho GTPase binding protein 1 | 158106 | 184428 | 182759 | 3, 31, 37 |
| 8 | RRM2B | ribonucleotide reductase regulatory TP53 inducible subunit M2B | 048392 | 104490 | 104517 | 22, 40, 29 |
| 8 | RPL30 | ribosomal protein L30 | 156482 | 132561 | 177459 | 31, 33, 24 |
| 8 | RPL7 | ribosomal protein L7 | 147604 | 274443 | 121039 | |
| 8 | RPL8 | ribosomal protein L8 | 161016 | 196378 | 197363 | 41, 21, 35 |
| 8 | RPS20 | ribosomal protein S20 | 008988 | 254087 | 172680 | 43, 11, 7 |
| 8 | RRS1 | ribosome biogenesis regulator homolog | 179041 | 147571 | 147576 | 21, 37, 9 |
| 8 | RNF122 | ring finger protein 122 | 133874 | 198042 | 133878 | 15, 40, 20 |
| 8 | RNF139 | ring finger protein 139 | 170881 | 183665 | 147687 | 26, 38, 39 |
| 8 | RNF170 | ring finger protein 170 | 120925 | 131931 | 168172 | 26, 6, 45 |
| 8 | RNF19A | ring finger protein 19A, RBR E3 ubiquitin protein ligase | 034677 | 104450 | 186106 | |
| 8 | RBM12B | RNA binding motif protein 12B | 183808 | 188343 | 164953 | 22, 12, 20 |
| 8 | RBPMS | RNA binding protein with multiple splicing | 157110 | 104671 | 197265 | 26, 27, 37 |
| 8 | POLR2K | RNA polymerase II subunit K | 147669 | 156509 | 104450 | 4, 18, 7 |
| 8 | POLR3D | RNA polymerase III subunit D | 168495 | 168490 | 197181 | 21, 26, 9 |
| 8 | RP1L1 | RP1 like 1 | 183638 | 184647 | 171060 | 14, |
| 8 | RP1 | RP1, axonemal microtubule associated | 104237 | 164736 | 206579 | |
| 8 | RSPO2 | R-spondin 2 | 147655 | 154188 | 104408 | 11, 31, 8 |
| 8 | RUNX1T1 | RUNX1 translocation partner 1 | 079102 | 147606 | 205133 | 8, 28, 15 |
| 8 | SGCZ | sarcoglycan zeta | 185053 | 164743 | 104723 | 20, |
| 8 | SPIDR | scaffolding protein involved in DNA repair | 164808 | 165102 | 221869 | 24, 13, 39 |
| 8 | SCARA3 | scavenger receptor class A member 3 | 168077 | 120885 | 147419 | 45, 6, 14 |
| 8 | SCARA5 | scavenger receptor class A member 5 | 168079 | 168078 | 189233 | 42, 10, 25 |
| 8 | SCX | scleraxis bHLH transcription factor | 260428 | 261236 | 185122 | |
| 8 | SCRT1 | scratch family transcriptional repressor 1 | 261678 | 185000 | 261587 | 14, 8, 38 |
| 8 | SCRIB | scribbled planar cell polarity protein | 180900 | 203499 | 179950 | 21, 35, 26 |
| 8 | SFRP1 | secreted frizzled related protein 1 | 104332 | 165061 | 147533 | 40, 15, 27 |
| 8 | SLURP1 | secreted LY6/PLAUR domain containing 1 | 126233 | 130193 | 197353 | |
| 8 | STK3 | serine/threonine kinase 3 | 104375 | 104361 | 156486 | |
| 8 | SGK3 | serum/glucocorticoid regulated kinase family member 3 | 104205 | 278888 | 178460 | 11, 31, 42 |
| 8 | SH2D4A | SH2 domain containing 4A | 104611 | 156011 | 147408 | 3, 37, 19 |
| 8 | SHARPIN | SHANK associated RH domain interactor | 179526 | 179091 | 179632 | 38, 34, 27 |
| 8 | SDR16C5 | short chain dehydrogenase/reductase family 16C member 5 | 170786 | 170791 | 181195 | 13, 30, 24 |
| 8 | SMIM18 | small integral membrane protein 18 | 253457 | 197265 | 104687 | 14, 20, 8 |
| 8 | SMIM19 | small integral membrane protein 19 | 176209 | 168575 | 147432 | 28, 6, 26 |
| 8 | SNAI2 | snail family transcriptional repressor 2 | 019549 | 034239 | 168333 | 35, 44, 9 |
| 8 | NKAIN3 | Sodium/potassium transporting ATPase interacting 3 | 185942 | 283485 | 137563 | 16, 10, 8 |
| 8 | SLC10A5 | solute carrier family 10 member 5 | 253598 | 133731 | 104231 | |
| 8 | SLC18A1 | solute carrier family 18 member A1 | 036565 | 175445 | 147416 | 26, 2, 36 |
| 8 | SLC20A2 | solute carrier family 20 member 2 | 168575 | 078668 | 176209 | 18, 40, 29 |
| 8 | SLC25A32 | solute carrier family 25 member 32 | 164933 | 164932 | 164934 | 45, 13, 31 |
| 8 | SLC25A37 | solute carrier family 25 member 37 | 147454 | 197217 | 167034 | 22, 27, 19 |
| 8 | SLC26A7 | solute carrier family 26 member 7 | 147606 | 214954 | 079102 | 40, 17, 18 |
| 8 | SLC30A8 | solute carrier family 30 member 8 | 164756 | 205002 | 164758 | 28, 38, 17 |
| 8 | SLC35G5 | solute carrier family 35 member G5 | 177710 | 104643 | 154319 | 33, |
| 8 | SLC39A14 | solute carrier family 39 member 14 | 104635 | 197181 | 120910 | 10, 40, 19 |
| 8 | SLC39A4 | solute carrier family 39 member 4 | 147804 | 071894 | 160948 | 17, 27, 35 |
| 8 | SLC45A4 | solute carrier family 45 member 4 | 022567 | 105339 | 204882 | 1, 16, 26 |
| 8 | SLC52A2 | solute carrier family 52 member 2 | 185803 | 271698 | 182325 | 37, 9, 34 |
| 8 | SLC7A13 | solute carrier family 7 member 13 | 164893 | 147613 | 123124 | 17, 42, |
| 8 | SLC7A2 | solute carrier family 7 member 2 | 003989 | 003987 | 104213 | 29, 9, 40 |
| 8 | SLCO5A1 | solute carrier organic anion transporter family member 5A1 | 137571 | 137573 | 147596 | 39, 36, 25 |
| 8 | SBSPON | somatomedin B and thrombospondin type 1 domain containing | 164764 | 147601 | 274443 | 11, 1, 32 |
| 8 | SORBS3 | sorbin and SH3 domain containing 3 | 120896 | 120910 | 120913 | 34, 26, 36 |
| 8 | SNX16 | sorting nexin 16 | 104497 | 164695 | 184672 | 1, 34, 13 |
| 8 | SNX31 | sorting nexin 31 | 174226 | 186106 | 070756 | 4, 31, 42 |
| 8 | SPAG1 | sperm associated antigen 1 | 104450 | 147669 | 034677 | 29, 16, 38 |
| 8 | SPAG11A | sperm associated antigen 11A | 178287 | 176782 | 176797 | 38, |
| 8 | SPAG11B | sperm associated antigen 11B | 164871 | 177243 | 177023 | 38, 14, |
| 8 | SPATC1 | spermatogenesis and centriole associated 1 | 186583 | 178719 | 178814 | 38, 8, |
| 8 | SOLE | squalene epoxidase | 104549 | 180938 | 164961 | 38, 7, 2 |
| 8 | SLA | Src like adaptor | 155926 | 042832 | 104415 | 39, 36, 40 |
| 8 | SOX17 | SRY-box 17 | 164736 | 137547 | 104237 | 27, 44, 45 |
| 8 | SOX7 | SRY-box 7 | 171056 | 171060 | 258724 | 45, 43, 11 |
| 8 | ST18 | ST18, C2H2C-type zinc finger | 147488 | 168300 | 196711 | 26, 31, 44 |
| 8 | ST3GAL1 | ST3 beta-galactoside alpha-2,3-sialyltransferase 1 | 008513 | 104419 | 066827 | |
| 8 | STC1 | stanniocalcin 1 | 159167 | 180053 | 042980 | 45, 17, 42 |
| 8 | STMN2 | stathmin 2 | 104435 | 104432 | 164683 | 26, 45, 8 |
| 8 | STMN4 | stathmin 4 | 015592 | 120907 | 104228 | 26, 8, 38 |
| 8 | STAU2 | staufen double-stranded RNA binding protein 2 | 040341 | 121039 | 258677 | 13, 43, 29 |
| 8 | SAMD12 | sterile alpha motif domain containing 12 | 177570 | 182197 | 164761 | 40, 42, 29 |
| 8 | STAR | steroidogenic acute regulatory protein | 147465 | 129691 | 175324 | 2, 25, 38 |
| 8 | SARAF | store-operated calcium entry associated regulatory factor | 133872 | 120875 | 104660 | 29, 18, 31 |
| 8 | SULF1 | sulfatase 1 | 137573 | 165084 | 137571 | 24, 1, 10 |
| 8 | SFTPC | surfactant protein C | 168484 | 168481 | 168487 | 10, 20, 15 |
| 8 | SDC2 | syndecan 2 | 169439 | 156471 | 104324 | |
| 8 | SDCBP | syndecan binding protein | 137575 | 167910 | 035681 | 5, 45, 29 |
| 8 | SYBU | syntabulin | 147642 | 147654 | 164794 | 8, 26, 1 |
| 8 | SNTB1 | syntrophin beta 1 | 172164 | 172167 | 170961 | 40, 2, 19 |
| 8 | SNTG1 | syntrophin gamma 1 | 147481 | 168333 | 147485 | 10, 8, 20 |
| 8 | TNKS | tankyrase | 173273 | 173281 | 175806 | 31, 38, 22 |
| 8 | TAF2 | TATA-box binding protein associated factor 2 | 064313 | 136960 | 136982 | 13, 24, 34 |
| 8 | TATDN1 | TatD DNase domain containing 1 | 147687 | 170881 | 147684 | 4, 34, 31 |
| 8 | TBC1D31 | TBC1 domain family member 31 | 156787 | 136986 | 147689 | 34, 39, 3 |
| 8 | TTI2 | TELO2 interacting protein 2 | 129696 | 172728 | 198042 | 1, 5, 31 |
| 8 | TERF1 | telomeric repeat binding factor 1 | 147601 | 182674 | 164764 | 12, 11, 33 |
| 8 | TDRP | testis development related protein | 180190 | 147364 | 104714 | 31, 32, 12 |
| 8 | TEX15 | testis expressed 15, meiosis and synapsis associated | 133863 | 104695 | 172733 | |
| 8 | THAP1 | THAP domain containing 1 | 131931 | 147434 | 120925 | 34, 22, 45 |
| 8 | THEM6 | thioesterase superfamily member 6 | 130193 | 160886 | 126233 | 9, 2, 37 |
| 8 | TOX | thymocyte selection associated high mobility group box | 198846 | 035681 | 178538 | 22, 39, 1 1 |
| 8 | TG | thyroglobulin | 042832 | 129292 | 155926 | 40, 18, 24 |
| 8 | TRHR | thyrotropin releasing hormone receptor | 174417 | 164841 | 120526 | |
| 8 | TIGD5 | tigger transposable element derived 5 | 179886 | 104529 | 104524 | 34, 33, 35 |
| 8 | TSTA3 | tissue specific transplantation antigen P35B | 104522 | 104524 | 183309 | 1, 16, 6 |
| 8 | TM2D2 | TM2 domain containing 2 | 169490 | 169495 | 168615 | 3, 6, 13 |
| 8 | TNFRSF10A | TNF receptor superfamily member 10a | 104689 | 173530 | 147457 | 27, 42, 39 |
| 8 | TNFRSF10B | TNF receptor superfamily member 10b | 120889 | 008853 | 173535 | 16, 42, 12 |
| 8 | TNFRSF10C | TNF receptor superfamily member 10c | 173535 | 120889 | 173530 | 5, 35, 17 |
| 8 | TNFRSF10D | TNF receptor superfamily member 10d | 173530 | 173535 | 104689 | 33, 12, 25 |
| 8 | TNFRSF11B | TNF receptor superfamily member 11b | 164761 | 177570 | 184374 | 45, 11, 40 |
| 8 | TONSL | tonsoku-like, DNA repair protein | 160949 | 160948 | 187954 | 34, 41, 12 |
| 8 | TOP1MT | topoisomerase (DNA) I, mitochondrial | 184428 | 185730 | 158106 | |
| 8 | TRAPPC9 | trafficking protein particle complex 9 | 167632 | 169427 | 104472 | 42, 24, |
| 8 | TCEA1 | transcription elongation factor A1 | 187735 | 147509 | 280473 | 40, 9, 7 |
| 8 | TCEB1 | Transcription elongation factor B subunit 1 | 154582 | 104343 | 175606 | |
| 8 | TCF24 | transcription factor 24 | 261787 | 178460 | 178125 | 27, 44, 12 |
| 8 | TRPS1 | transcriptional repressor GATA binding 1 | 104447 | 164796 | 147677 | |
| 8 | TACC1 | transforming acidic coiled-coil containing protein 1 | 147526 | 196166 | 169499 | 45, 29, 22 |
| 8 | TRPA1 | transient receptor potential cation channel subfamily A member 1 | 104321 | 178860 | 182674 | 24, 25, 10 |
| 8 | TRAM1 | translocation associated membrane protein 1 | 067167 | 140396 | 147592 | 43, 3, 4 |
| 8 | TMEM249 | transmembrane protein 249 | 261587 | 261678 | 271698 | 38, 17, 16 |
| 8 | TMEM55A | transmembrane protein 55A | 155099 | 253250 | 155100 | 45, 11, 14 |
| 8 | TMEM64 | transmembrane protein 64 | 180694 | 104327 | 123119 | |
| 8 | TMEM65 | transmembrane protein 65 | 164983 | 214814 | 183665 | 36, 4, 27 |
| 8 | TMEM67 | transmembrane protein 67 | 164953 | 183808 | 164951 | 42, 13, 38 |
| 8 | TMEM68 | transmembrane protein 68 | 167904 | 206579 | 137574 | 13, 29, 45 |
| 8 | TMEM70 | transmembrane protein 70 | 175606 | 154582 | 154589 | 22, 20, 29 |
| 8 | TMEM71 | transmembrane protein 71 | 165071 | 129295 | 129292 | 5, 39, 7 |
| 8 | TMEM74 | transmembrane protein 74 | 164841 | 104412 | 174417 | 8, 14, |
| 8 | TRIB1 | tibbles pseudokinase 1 | 173334 | 156831 | 168672 | 40, 29, 26 |
| 8 | TGS1 | trimethylguanosine synthase 1 | 137574 | 167904 | 254087 | 1, 45, 23 |
| 8 | TRIM35 | tripartite motif containing 35 | 104228 | 015592 | 120899 | 1, 41, 34 |
| 8 | TRIM55 | tripartite motif containing 55 | 147573 | 147570 | 147571 | 10, 15, 25 |
| 8 | TRIQK | triple QxxK/R motif containing | 205133 | 079102 | 188343 | 42, 6, 32 |
| 8 | TRMT12 | tRNA methyltransferase 12 homolog | 183665 | 164983 | 170881 | 27, 45, 17 |
| 8 | TSNARE1 | t-SNARE domain containing 1 | 171045 | 226490 | 181790 | 26, 22, 38 |
| 8 | TSPYL5 | TSPY like 5 | 180543 | 104324 | 147649 | 38, 11, 15 |
| 8 | TPD52 | tumor protein D52 | 076554 | 276418 | 205189 | 17, |
| 8 | TP53INP1 | tumor protein p53 inducible nuclear protein 1 | 164938 | 156170 | 175895 | 39, 22, 4 |
| 8 | TUSC3 | tumor suppressor candidate 3 | 104723 | 185053 | 038945 | |
| 8 | YWHAZ | tyrosine 3-monooxygenase/tryptophan 5-monooxygenase activation protein zeta | 164924 | 070756 | 120963 | 1, 12, 3 |
| 8 | SGK223 | Tyrosine-protein kinase SgK223 | 275342 | 225327 | 253958 | 7, 12, 41 |
| 8 | UQCRB | ubiquinol-cytochrome c reductase binding protein | 156467 | 156466 | 156469 | 11, 25, 6 |
| 8 | UBE2V2 | ubiquitin conjugating enzyme E2 V2 | 169139 | 104738 | 034239 | 4, 34, 7 |
| 8 | UBE2W | ubiquitin conjugating enzyme E2 W | 104343 | 258677 | 154582 | 26, 4, 13 |
| 8 | UBR5 | ubiquitin protein ligase E3 component n-recognin 5 | 104517 | 048392 | 155087 | 11, 18, 10 |
| 8 | USP17L1 | ubiquitin specific peptidase 17-like family member 1 | 230549 | 164816 | 236125 | |
| 8 | USP17L2 | ubiquitin specific peptidase 17-like family member 2 | 223443 | 226430 | 186523 | |
| 8 | USP17L3 | ubiquitin specific peptidase 17-like family member 3 | 225327 | 237038 | 275342 | |
| 8 | USP17L4 | ubiquitin specific peptidase 17-like family member 4 | 236125 | 230549 | 215372 | |
| 8 | USP17L7 | ubiquitin specific peptidase 17-like family member 7 | 226430 | 215343 | 223443 | |
| 8 | USP17L8 | ubiquitin specific peptidase 17-like family member 8 | 237038 | 215356 | 225327 | |
| 8 | UBXN2B | UBX domain protein 2B | 215114 | 169122 | 167910 | 11, 40, 5 |
| 8 | UBXN8 | UBX domain protein 8 | 104691 | 104687 | 104695 | |
| 8 | UNC5D | unc-5 netrin receptor D | 156687 | 133878 | 215262 | 14, 8, 12 |
| 8 | UTP23 | UTP23, small subunit processome component | 147679 | 147677 | 164754 | 42, 35, 23 |
| 8 | VPS13B | vacuolar protein sorting 13 homolog B | 132549 | 164920 | 164919 | 1, 18, 33 |
| 8 | VCPIP1 | valosin containing protein interacting protein 1 | 175073 | 185697 | 270024 | 22, 26, 39 |
| 8 | MYC | v-myc avian myelocytomatosis viral oncogene homolog | 136997 | 279524 | 280151 | 7, 4, 5 |
| 8 | VDAC3 | voltage dependent anion channel 3 | 078668 | 104371 | 168575 | 30, 15, 26 |
| 8 | VPS28 | VPS28, ESCRT-I subunit | 160948 | 147804 | 160949 | 29, 27, 15 |
| 8 | VPS37A | VPS37A, ESCRT-I subunit | 155975 | 198791 | 003987 | |
| 8 | WDR97 | WD repeat domain 97 | 179698 | 179632 | 235173 | 38, 36, 39 |
| 8 | WDYHV1 | WDYHV motif containing 1 | 156795 | 156802 | 156804 | 18, 11, 2 |
| 8 | WRN | Werner syndrome RecQ like helicase | 165392 | 172733 | 157168 | 18, 1, 11 |
| 8 | WISP1 | WNT1 inducible signaling pathway protein 1 | 104415 | 155926 | 104419 | 1, 11, 10 |
| 8 | WHSC1L1 | Wolf-Hirschhorn syndrome candidate 1-like 1 | 147548 | 147535 | 165046 | 22, 6, 40 |
| 8 | WWP1 | WW domain containing E3 ubiquitin protein ligase 1 | 123124 | 164893 | 176623 | 11, 23, 1 |
| 8 | XKR4 | XK related 4 | 206579 | 104237 | 167904 | 26, 14, 8 |
| 8 | XKR5 | XK related 5 | 275591 | 155189 | 164825 | 21, |
| 8 | XKR6 | XK related 6 | 171044 | 254093 | 280273 | |
| 8 | XKR9 | XK related 9 | 221947 | 147592 | 104313 | 16, 38, 25 |
| 8 | YTHDF3 | YTH N6-methyladenosine RNA binding protein 3 | 185728 | 137561 | 180828 | 1, 22, 10 |
| 8 | ZFP41 | ZFP41 zinc finger protein | 181638 | 277494 | 264668 | 11, 4, 26 |
| 8 | ZHX1-C8orf76 | ZHX1-C8orf76 readthrough | 259305 | 189376 | 165156 | |
| 8 | ZFAND1 | zinc finger AN1-type containing 1 | 104231 | 253598 | 164695 | 34, 29, 31 |
| 8 | ZFAT | zinc finger and AT-hook domain containing | 066827 | 008513 | 131773 | 43, 32, 1 |
| 8 | ZBTB10 | zinc finger and BTB domain containing 10 | 205189 | 076554 | 164684 | 26, 25, 33 |
| 8 | ZC2HC1A | zinc finger C2HC-type containing 1A | 104427 | 171033 | 104432 | 30, 22, 26 |
| 8 | ZC3H3 | zinc finger CCCH-type containing 3 | 014164 | 182759 | 104518 | 34, 9, 27 |
| 8 | ZDHHC2 | zinc finger DHHC-type containing 2 | 104219 | 155970 | 198791 | 31, 28, 14 |
| 8 | ZFHX4 | zinc finger homeobox 4 | 091656 | 164749 | 164751 | 19, 18, 38 |
| 8 | ZMAT4 | zinc finger matrin-type 4 | 165061 | 176907 | 104332 | 40, 31, 14 |
| 8 | ZNF16 | zinc finger protein 16 | 170631 | 196150 | 182307 | 26, 24, 9 |
| 8 | ZNF250 | zinc finger protein 250 | 196150 | 170619 | 170631 | 31, 15, 27 |
| 8 | ZNF251 | zinc finger protein 251 | 198169 | 147799 | 196378 | 29, 24, 36 |
| 8 | ZNF34 | zinc finger protein 34 | 196378 | 198169 | 161016 | 1, 15, 14 |
| 8 | ZNF395 | zinc finger protein 395 | 186918 | 168081 | 214050 | 41, 22, 18 |
| 8 | ZNF517 | zinc finger protein 517 | 197363 | 161016 | 147789 | 19, 13, 21 |
| 8 | ZNF572 | zinc finger protein 572 | 180938 | 170873 | 104549 | 24, 27, 38 |
| 8 | ZNF596 | zinc finger protein 596 | 172748 | 176269 | 147364 | 18, 38, 36 |
| 8 | ZNF623 | zinc finger protein 623 | 183309 | 104522 | 181135 | |
| 8 | ZNF696 | zinc finger protein 696 | 185730 | 250571 | 184428 | 42, 27, 23 |
| 8 | ZNF7 | zinc finger protein 7 | 147789 | 197363 | 170619 | 1, 4, 25 |
| 8 | ZNF703 | zinc finger protein 703 | 183779 | 215262 | 147475 | 26, 40, 37 |
| 8 | ZNF704 | zinc finger protein 704 | 164684 | 205189 | 076641 | |
| 8 | ZNF705B | zinc finger protein 705B | 215356 | 171711 | 237038 | 38, |
| 8 | ZNF705D | zinc finger protein 705D | 215343 | 233050 | 226430 | 35, |
| 8 | ZNF705G | zinc finger protein 705G | 215372 | 236125 | 177257 | |
| 8 | ZNF706 | zinc finger protein 706 | 120963 | 164924 | 083307 | 30, 11, 32 |
| 8 | ZNF707 | zinc finger protein 707 | 181135 | 183309 | 255181 | |
| 8 | ZFPM2 | zinc finger protein, FOG family member 2 | 169946 | 147650 | 164830 | 26, 15, 38 |
| 8 | ZHX1 | zinc fingers and homeoboxes 1 | 165156 | 259305 | 156802 | 42, 24, 4 |
| 8 | ZHX2 | zinc fingers and homeoboxes 2 | 178764 | 170961 | 136986 | 22, 21, 23 |
| 9 | AGPAT2 | 1-acylglycerol-3-phosphate O-acyltransferase 2 | 169692 | 172889 | 165716 | 3, 9, 34 |
| 9 | HACD4 | 3-hydroxyacyl-CoA dehydratase 4 | 188921 | 188352 | 171855 | |
| 9 | ABHD17B | abhydrolase domain containing 17B | 107362 | 135048 | 281760 | 39, 29, 22 |
| 9 | ABL1 | ABL proto-oncogene 1, non-receptor tyrosine kinase | 097007 | 130713 | 188710 | 25, 35, 43 |
| 9 | ABO | ABO, alpha 1-3-N-acetylgalactosaminyltransferase and alpha 1-3- | 175164 | 171102 | 148296 | 13, 2, 34 |
| | | galactosyltransferase | | | | |
| 9 | ANP32B | acidic nuclear phosphoprotein 32 family member B | 136938 | 136929 | 095380 | 41, 34, 11 |
| 9 | ACO1 | aconitase 1 | 122729 | 174482 | 107201 | 1, 42, 40 |
| 9 | ACTL7A | actin like 7A | 187003 | 148156 | 070061 | 38, 8, |
| 9 | ACTL7B | actin like 7B | 148156 | 136826 | 187003 | 38, 8, |
| 9 | ARPC5L | actin related protein 2/3 complex subunit 5 like | 136950 | 136942 | 136935 | 31, 41, 20 |
| 9 | ADAMTS13 | ADAM metallopeptidase with thrombospondin type 1 motif 13 | 160323 | 148300 | 160325 | 26, 16, 38 |
| 9 | ADAMTSL1 | ADAMTS like 1 | 178031 | 107295 | 155875 | |
| 9 | ADAMTSL2 | ADAMTS like 2 | 197859 | 187616 | 196990 | 26, 27, 23 |
| 9 | AK1 | adenylate kinase 1 | 106992 | 106991 | 160408 | 25, 34, 15 |
| 9 | AK3 | adenylate kinase 3 | 147853 | 106993 | 120158 | 29, 2, 15 |
| 9 | AK8 | adenylate kinase 8 | 165695 | 125484 | 165698 | 38, 13, 10 |
| 9 | ADGRD2 | adhesion G protein-coupled receptor D2 | 180264 | 136930 | 136931 | |
| 9 | AAED1 | AhpC/TSA antioxidant enzyme domain containing 1 | 158122 | 081377 | 081386 | 10, 20, 6 |
| 9 | AKAP2 | A-kinase anchoring protein 2 | 241978 | 157654 | 188959 | 42, 1, 45 |
| 9 | ALDH1A1 | aldehyde dehydrogenase 1 family member A1 | 165092 | 165091 | 135046 | 42, 2, 19 |
| 9 | ALDH1B1 | aldehyde dehydrogenase 1 family member B1 | 137124 | 107338 | 137142 | 3, 13, 1 |
| 9 | ALDOB | aldolase, fructose-bisphosphate B | 136872 | 136870 | 165152 | 19, 36, 17 |
| 9 | ALG2 | ALG2, alpha-1,3/1,6-mannosyltransferase | 119523 | 106799 | 106803 | 13, 42, 17 |
| 9 | ACER2 | alkaline ceramidase 2 | 177076 | 137154 | 155886 | 29, 24, 42 |
| 9 | AIF1L | allograft inflammatory factor 1 like | 126878 | 050555 | 126883 | 36, 8, 42 |
| 9 | AMBP | alpha-1-microglobulin/bikunin precursor | 106927 | 157657 | 136883 | 19, 36, 31 |
| 9 | ALAD | aminolevulinate dehydratase | 148218 | 119431 | 148229 | 2, 39, 8 |
| 9 | APBA1 | amyloid beta precursor protein binding family A member 1 | 107282 | 135063 | 188647 | |
| 9 | ANAPC2 | anaphase promoting complex subunit 2 | 176248 | 185863 | 176101 | 21, 26, 5 |
| 9 | ANGPTL2 | angiopoietin like 2 | 136859 | 136828 | 136895 | 1, 26, 10 |
| 9 | ASB6 | ankyrin repeat and SOCS box containing 6 | 148331 | 179058 | 167157 | 27, 17, 26 |
| 9 | ANKS6 | ankyrin repeat and sterile alpha motif domain containing 6 | 165138 | 136928 | 119514 | 3, 23, 26 |
| 9 | ANKRD18A | ankyrin repeat domain 18A | 180071 | 283486 | 283329 | 5, 3, 33 |
| 9 | ANKRD18B | ankyrin repeat domain 18B | 230453 | 165271 | 205274 | 19, 38, 28 |
| 9 | ANKRD20A1 | ankyrin repeat domain 20 family member A1 | 260691 | 275969 | 196873 | |
| 9 | ANKRD20A2 | ankyrin repeat domain 20 family member A2 | 183148 | 204849 | 273514 | 36, 22, 25 |
| 9 | ANKRD20A3 | ankyrin repeat domain 20 family member A3 | 276203 | 184659 | 274349 | |
| 9 | ANKRD20A4 | ankyrin repeat domain 20 family member A4 | 172014 | 283378 | 204779 | 27, 33, |
| 9 | ANXA1 | annexin A1 | 135046 | 165092 | 198963 | 30, 3, 18 |
| 9 | APTX | aprataxin | 137074 | 188133 | 086061 | 34, 9, 21 |
| 9 | AQP3 | aquaporin 3 (Gill blood group) | 165272 | 165269 | 165271 | 34, 13, 39 |
| 9 | AQP7 | aquaporin 7 | 165269 | 086102 | 165272 | 1, 23, 15 |
| 9 | ASS1 | argininosuccinate synthase 1 | 130707 | 148357 | 107164 | 9, 25, 16 |
| 9 | ARRDC1 | arrestin domain containing 1 | 197070 | 165724 | 181090 | 9, 5, 32 |
| 9 | ASPN | asporin | 106819 | 127083 | 106823 | 45, 13, 44 |
| 9 | ASTN2 | astrotactin 2 | 148219 | 182752 | 119401 | 31, 1, 14 |
| 9 | AKNA | AT-hook transcription factor | 106948 | 228278 | 095397 | 22, 1, 7 |
| 9 | ABCA1 | ATP binding cassette subfamily A member 1 | 165029 | 165028 | 070214 | 11, 1, 26 |
| 9 | ABCA2 | ATP binding cassette subfamily A member 2 | 107331 | 169583 | 180549 | 8, 26, 37 |
| 9 | AGTPBP1 | ATP/GTP binding protein 1 | 135049 | 148053 | 135040 | 10, 40, 8 |
| 9 | ATP6V1G1 | ATPase H+ transporting V1 subunit G1 | 136888 | 095397 | 157693 | 31, 29, 4 |
| 9 | ARID3C | AT-rich interaction domain 3C | 205143 | 137100 | 147955 | 41, |
| 9 | AUH | AU RNA binding methylglutaconyl-CoA hydratase | 148090 | 165025 | 165030 | 31, 29, 10 |
| 9 | BARHL1 | BarH like homeobox 1 | 125492 | 188523 | 125485 | 8, |
| 9 | BARX1 | BARX homeobox 1 | 131668 | 197724 | 158079 | 9, 37, 13 |
| 9 | BNC2 | basonuclin 2 | 173068 | 205549 | 044459 | 15, 44, 1 |
| 9 | BSPRY | B-box and SPRY domain containing | 119411 | 148225 | 119431 | 29, 40, 3 |
| 9 | BAG1 | BCL2 associated athanogene 1 | 107262 | 122711 | 086065 | 9, 44, 34 |
| 9 | B4GALT1 | beta-1,4-galactosyltransferase 1 | 086062 | 122692 | 122711 | 29, 22, 1 |
| 9 | BICD2 | BICD cargo adaptor 2 | 185963 | 127080 | 127081 | 29, 22, 11 |
| 9 | BAAT | bile acid-CoA: amino acid N-acyltransferase | 136881 | 148123 | 136897 | 19, 4, 35 |
| 9 | BRINP1 | BMP/retinoic acid inducible neural specific 1 | 078725 | 136869 | 136861 | 18, 7, 25 |
| 9 | BRD3 | bromodomain containing 3 | 169925 | 235106 | 196363 | 9, 22, 40 |
| 9 | CACFD1 | calcium channel flower domain containing 1 | 160325 | 160323 | 160326 | 29, 42, 38 |
| 9 | CACNA1B | calcium voltage-gated channel subunit alpha1 B | 148408 | 181090 | 107175 | 31, 26, 13 |
| 9 | CCIN | calicin | 185972 | 122694 | 122705 | 38, 8, 33 |
| 9 | CAMSAP1 | calmodulin regulated spectrin associated protein 1 | 130559 | 107147 | 130560 | 31, 14, 12 |
| 9 | CREB3 | cAMP responsive element binding protein 3 | 107175 | 137076 | 070610 | 34, 9, 27 |
| 9 | CA9 | carbonic anhydrase 9 | 107159 | 137135 | 198467 | 9, 3, 1 |
| 9 | CEL | carboxyl ester lipase | 170835 | 148308 | 160271 | |
| 9 | CRAT | carnitine O-acetyltransferase | 095321 | 167130 | 119383 | 7, 34, 35 |
| 9 | CARNMT1 | carnosine N-methyltransferase 1 | 156017 | 135045 | 106733 | 22, 26, 45 |
| 9 | CAAP1 | caspase activity and apoptosis inhibitor 1 | 120159 | 198680 | 137055 | 9, 43, 13 |
| 9 | CARD19 | caspase recruitment domain family member 19 | 165233 | 157303 | 131669 | 29, 36, 6 |
| 9 | CARD9 | caspase recruitment domain family member 9 | 187796 | 213221 | 165684 | 7, 36, 13 |
| 9 | CTNNAL1 | catenin alpha like 1 | 119326 | 119328 | 106771 | 11, 2, 45 |
| 9 | CTSL | cathepsin L | 135047 | 196730 | 177992 | 32, 29, 6 |
| 9 | CTSV | cathepsin V | 136943 | 188152 | 197816 | 39, 12, 13 |
| 9 | CCL19 | C-C motif chemokine ligand 19 | 172724 | 187186 | 137077 | 30, 45, 36 |
| 9 | CCL21 | C-C motif chemokine ligand 21 | 137077 | 172724 | 205108 | 45, 15, 22 |
| 9 | CCL27 | C-C motif chemokine ligand 27 | 213927 | 137070 | 187186 | 14, 32, 20 |
| 9 | CD274 | CD274 molecule | 120217 | 107020 | 197646 | 32, 39, 42 |
| 9 | CD72 | CD72 molecule | 137101 | 107140 | 137078 | 41, 36, 21 |
| 9 | CKS2 | CDC28 protein kinase regulatory subunit 2 | 123975 | 148082 | 187742 | 3, 12, 2 |
| 9 | CDK5RAP2 | CDK5 regulatory subunit associated protein 2 | 136861 | 078725 | 106780 | 29, 42, 1 |
| 9 | CIZ1 | CDKN1A interacting zinc finger protein 1 | 148337 | 171159 | 106976 | 34, 3, 9 |
| 9 | CDC14B | cell division cycle 14B | 081377 | 130956 | 158122 | 11, 2, 24 |
| 9 | CDC26 | cell division cycle 26 | 176386 | 136868 | 136875 | 31, 1, 43 |
| 9 | CDC37L1 | cell division cycle 37 like 1 | 106993 | 205808 | 147853 | 15, 26, 31 |
| 9 | CNTLN | centlein | 044459 | 173068 | 107295 | 6, 26, 13 |
| 9 | CNTRL | centriolin | 119397 | 106804 | 119396 | 22, 25, 39 |
| 9 | CENPP | centromere protein P | 188312 | 198000 | 106809 | 24, 21, 37 |
| 9 | CEP78 | centrosomal protein 78 | 148019 | 156052 | 135069 | 21, 37, 6 |
| 9 | CER1 | cerberus 1, DAN family BMP antagonist | 147869 | 175893 | 164946 | 12, 28, 27 |
| 9 | CERCAM | cerebral endothelial cell adhesion molecule | 167123 | 167118 | 136811 | 32, 27, 28 |
| 9 | CHMP5 | charged multivesicular body protein 5 | 086065 | 107262 | 086102 | 4, 7, 34 |
| 9 | CLIC3 | chloride intracellular channel 3 | 169583 | 148362 | 107331 | 32, 20, 15 |
| 9 | C9orf116 | chromosome 9 open reading frame 116 | 160345 | 196422 | 122140 | 17, 2, 6 |
| 9 | C9orf129 | chromosome 9 open reading frame 129 | 204352 | 165238 | 188938 | 33, 28, 8 |
| 9 | C9orf131 | chromosome 9 open reading frame 131 | 174038 | 137094 | 165280 | 38, 18, 30 |
| 9 | C9orf135 | chromosome 9 open reading frame 135 | 204711 | 188647 | 165072 | 10, 30, 38 |
| 9 | C9orf142 | chromosome 9 open reading frame 142 | 148362 | 214402 | 169583 | 9, 20, 27 |
| 9 | C9orf152 | chromosome 9 open reading frame 152 | 188959 | 241978 | 136810 | 42, 4, 23 |
| 9 | C9orf153 | chromosome 9 open reading frame 153 | 187753 | 135052 | 135070 | 38, 10, 8 |
| 9 | C9orf16 | chromosome 9 open reading frame 16 | 171159 | 148346 | 148337 | 2, 14, 34 |
| 9 | C9orf172 | chromosome 9 open reading frame 172 | 232434 | 196642 | 054148 | 17, 23, 8 |
| 9 | C9orf24 | chromosome 9 open reading frame 24 | 164972 | 164976 | 164970 | 38, 36, 8 |
| 9 | C9orf3 | chromosome 9 open reading frame 3 | 148120 | 165140 | 158169 | 34, 40, 6 |
| 9 | C9orf40 | chromosome 9 open reading frame 40 | 135045 | 119121 | 156017 | 22, 9, 38 |
| 9 | C9orf43 | chromosome 9 open reading frame 43 | 157653 | 148229 | 138835 | 38, 9, 44 |
| 9 | C9orf47 | chromosome 9 open reading frame 47 | 186354 | 130045 | 213694 | |
| 9 | C9orf50 | chromosome 9 open reading frame 50 | 179058 | 148335 | 148331 | 38, 37, 27 |
| 9 | C9orf57 | chromosome 9 open reading frame 57 | 204669 | 155621 | 119125 | 38, 16, |
| 9 | C9orf64 | chromosome 9 open reading frame 64 | 165118 | 165115 | 165119 | 30, 21, 4 |
| 9 | C9orf66 | chromosome 9 open reading frame 66 | 183784 | 172785 | 107099 | 17, 33, 38 |
| 9 | C9orf69 | chromosome 9 open reading frame 69 | 238227 | 148411 | 107187 | 27, 9, 14 |
| 9 | C9orf72 | chromosome 9 open reading frame 72 | 147894 | 147896 | 174482 | 31, 30, 5 |
| 9 | C9orf78 | chromosome 9 open reading frame 78 | 136819 | 136827 | 136878 | 30, 5, 7 |
| 9 | C9orf84 | chromosome 9 open reading frame 84 | 165181 | 242616 | 148154 | 38, 37, 23 |
| 9 | C9orf85 | chromosome 9 open reading frame 85 | 155621 | 281760 | 204669 | 7, 45, 42 |
| 9 | C9orf92 | chromosome 9 open reading frame 92 | 205549 | 164989 | 173068 | 10, 17, |
| 9 | CFAP157 | cilia and flagella associated protein 157 | 160401 | 187024 | 167094 | 9, 38, 44 |
| 9 | CFAP77 | cilia and flagella associated protein 77 | 188523 | 125482 | 125492 | 38, 44, |
| 9 | CNTFR | ciliary neurotrophic factor receptor | 122756 | 168913 | 164967 | 26, 14, 8 |
| 9 | CLTA | clathrin light chain A | 122705 | 185972 | 159921 | 21, 34, 35 |
| 9 | CBWD1 | COBW domain containing 1 | 172785 | 170122 | 183784 | |
| 9 | CBWD3 | COBW domain containing 3 | 196873 | 260691 | 187559 | |
| 9 | CBWD5 | COBW domain containing 5 | 147996 | 204779 | 184659 | |
| 9 | CBWD7 | COBW domain containing 7 | 215126 | 273514 | 154529 | |
| 9 | COQ4 | coenzyme Q4 | 167113 | 167112 | 167114 | 31, 9, 21 |
| 9 | CCDC107 | coiled-coil domain containing 107 | 159884 | 137078 | 137135 | 39, 36, 28 |
| 9 | CCDC171 | coiled-coil domain containing 171 | 164989 | 164985 | 205549 | 10, 6, 38 |
| 9 | CCDC180 | coiled-coil domain containing 180 | 197816 | 136943 | 196116 | 38, 42, 24 |
| 9 | CCDC183 | coiled-coil domain containing 183 | 213213 | 272896 | 196642 | 17, 20, 38 |
| 9 | CCDC187 | coiled-coil domain containing 187 | 260220 | 279813 | 160360 | 38, 20, 8 |
| 9 | COL5A1 | collagen type V alpha 1 chain | 130635 | 186350 | 160339 | 12, 6, 11 |
| 9 | COL15A1 | collagen type XV alpha 1 chain | 204291 | 119514 | 106799 | |
| 9 | COL27A1 | collagen type XXVII alpha 1 chain | 196739 | 136883 | 229314 | 11, 43, 26 |
| 9 | C5 | complement C5 | 106804 | 056558 | 119397 | 19, 41, 17 |
| 9 | C8G | complement C8 gamma chain | 176919 | 159069 | 184925 | 37, 19, 38 |
| 9 | CNTNAP3 | contactin associated protein-like 3 | 106714 | 283329 | 204849 | 26, 45, 5 |
| 9 | CNTNAP3B | contactin associated protein-like 3B | 154529 | 215126 | 185775 | |
| 9 | CORO2A | coronin 2A | 106789 | 106785 | 095383 | 3, 24, 33 |
| 9 | CRB2 | crumbs 2, cell polarity complex component | 148204 | 165209 | 119522 | 44, 8, 38 |
| 9 | CDK20 | cyclin dependent kinase 20 | 156345 | 283205 | 106723 | 2, 38, 42 |
| 9 | CDK9 | cyclin dependent kinase 9 | 136807 | 279363 | 136877 | 40, 26, 9 |
| 9 | CDKN2A | cyclin dependent kinase inhibitor 2A | 147889 | 264545 | 147883 | 9, 34, 44 |
| 9 | CDKN2B | cyclin dependent kinase inhibitor 2B | 147883 | 147889 | 176399 | 24, 4, 36 |
| 9 | CYLC2 | cylicin 2 | 155833 | 188386 | 136824 | 38, |
| 9 | CYSRT1 | cysteine rich tail 1 | 197191 | 212864 | 233198 | 19, 12, 8 |
| 9 | DAB2IP | DAB2 interacting protein | 136848 | 148175 | 175764 | |
| 9 | DCAF10 | DDB1 and CUL4 associated factor 10 | 122741 | 107371 | 122696 | 41, 18, 38 |
| 9 | DCAF12 | DDB1 and CUL4 associated factor 12 | 198876 | 137073 | 165006 | 38, 42, 24 |
| 9 | DDX31 | DEAD-box helicase 31 | 125485 | 125492 | 125484 | 9, 1, 33 |
| 9 | DAPK1 | death associated protein kinase 1 | 196730 | 180447 | 135047 | 32, 29, 39 |
| 9 | DOCK8 | dedicator of cytokinesis 8 | 107099 | 183784 | 107104 | 22, 26, 39 |
| 9 | DEC1 | deleted in esophageal cancer 1 | 173077 | 041982 | 182752 | 6, 35, |
| 9 | DENND1A | DENN domain containing 1A | 119522 | 148204 | 106689 | 26, 9, 31 |
| 9 | DENND4C | DENN domain containing 4C | 137145 | 147872 | 137154 | 11, 1, 4 |
| 9 | DDX58 | DExD/H-box helicase 58 | 107201 | 122729 | 197579 | 13, 23, 24 |
| 9 | DPP7 | dipeptidyl peptidase 7 | 176978 | 177239 | 176884 | 9, 1, 35 |
| 9 | DPH7 | diphthamide biosynthesis 7 | 148399 | 182154 | 165724 | 4, 39, 16 |
| 9 | DIRAS2 | DIRAS family GTPase 2 | 165023 | 130222 | 165025 | 8, 14, 23 |
| 9 | DMRTA1 | DMRT like family A1 | 176399 | 147883 | 107105 | 17, 42, 19 |
| 9 | POLE3 | DNA polymerase epsilon 3, accesspry subunit | 148229 | 148218 | 157653 | 2, 9, 21 |
| 9 | SMU1 | DNA replication regulator and spliceosomal factor | 122692 | 086061 | 086062 | 40, 20, 1 |
| 9 | DNAJA1 | DnaJ heat shock protein family (Hsp40) member A1 | 086061 | 137074 | 122692 | 41, 30, 1 |
| 9 | DNAJB5 | DnaJ heat shock protein family (Hsp40) member B5 | 137094 | 122733 | 174038 | 18, 25, 31 |
| 9 | DNAJC25 | DnaJ heat shock protein family (Hsp40) member C25 | 059769 | 106853 | 244115 | 28, 17, 7 |
| 9 | DNAJC25- | DNAJC25-GNG10 readthrough | 244115 | 059769 | 242616 | |
| | GNG10 | | | | | |
| 9 | DNLZ | DNL-type zinc finger | 213221 | 160360 | 187796 | 34, 33, 17 |
| 9 | DOLK | dolichol kinase | 175283 | 251184 | 095319 | 41, 27, 25 |
| 9 | DOLPP1 | dolichyldiphosphatase 1 | 167130 | 148343 | 095321 | 9, 26, 27 |
| 9 | DPM2 | dolichyl-phosphate mannosyltransferase subunit 2, regulatory | 136908 | 167103 | 167106 | 34, 9, 27 |
| 9 | DBH | dopamine beta-hydroxylase | 123454 | 196990 | 123453 | 26, 2, 8 |
| 9 | DMRT1 | doublesex and mab-3 related transcription factor 1 | 137090 | 107104 | 064218 | 10, 38, 8 |
| 9 | DMRT2 | doublesex and mab-3 related transcription factor 2 | 173253 | 064218 | 080503 | 29, 15, 17 |
| 9 | DMRT3 | doublesex and mab-3 related transcription factor 3 | 064218 | 137090 | 173253 | |
| 9 | DCTN3 | dynactin subunit 3 | 137100 | 164967 | 205143 | 34, 43, 41 |
| 9 | DNM1 | dynamin 1 | 106976 | 148337 | 167110 | 26, 8, 29 |
| 9 | DNAI1 | dynein axenemal intermediate chain 1 | 122735 | 164970 | 168913 | 38, 42, 30 |
| 9 | ENTPD2 | ectonucleoside triphosphate diphosphohydrolase 2 | 054179 | 107281 | 186193 | 26, 3, 27 |
| 9 | ENTPD8 | ectonucleoside triphosphate diphosphohydrolase 8 | 188833 | 188747 | 165802 | 3, 28, 16 |
| 9 | EGFL7 | EGF like domain multiple 7 | 172889 | 148400 | 169692 | 32, 17, 15 |
| 9 | ELAVL2 | ELAV like RNA binding protein 2 | 107105 | 176399 | 205442 | 38, 14, 8 |
| 9 | ENG | endoglin | 106991 | 136877 | 106992 | 45, 36, 39 |
| 9 | ENDOG | endonuclease G | 167136 | 107021 | 198917 | 41, 34, 33 |
| 9 | ERMP1 | endoplasmic reticulum metallopeptidase 1 | 099219 | 107036 | 183354 | 29, 24, 4 |
| 9 | ERP44 | endoplasmic reticulum protein 44 | 023318 | 136874 | 119509 | 31, 29, 32 |
| 9 | EDF1 | endothelial differentiation related factor 1 | 107223 | 177943 | 127191 | 43, 33, 18 |
| 9 | ENHO | energy homeostasis associated | 168913 | 122735 | 122756 | 26, 8, 38 |
| 9 | EQTN | equatorin | 120160 | 120156 | 120162 | 38, 20, |
| 9 | ERCC6L2 | ERCC excision repair 6 like 2 | 182150 | 185920 | 130948 | |
| 9 | EPB41L4B | erythrocyte membrane protein band 4.1 like 4B | 095203 | 260230 | 070159 | 26, 16, 4 |
| 9 | EHMT1 | euchromatic histone lysine methyltransferase 1 | 181090 | 197070 | 148408 | 21, 22, 41 |
| 9 | EXD3 | exonuclease 3′-5′ domain containing 3 | 187609 | 198435 | 188747 | 36, 6, 32 |
| 9 | EXOSC2 | exosome component 2 | 130713 | 130711 | 097007 | 1, 34, 43 |
| 9 | EXOSC3 | exosome component 3 | 107371 | 165275 | 122741 | |
| 9 | ECM2 | extracellular matrix protein 2 | 106823 | 106819 | 127080 | 45, 26, 6 |
| 9 | FAM102A | family with sequence similarity 102 member A | 167106 | 136908 | 171169 | 22, 28, 31 |
| 9 | FAM120A | family with sequence similarity 120A | 048828 | 188938 | 197724 | 11, 22, 6 |
| 9 | FAM120AOS | family with sequence similarity 120A opposite strand | 188938 | 204352 | 048828 | 29, 20, 16 |
| 9 | FAM122A | family with sequence similarity 122A | 187866 | 107242 | 165059 | 42, 1, 44 |
| 9 | FAM129B | family with sequence similarity 129 member B | 136830 | 148356 | 136854 | 9, 6, 33 |
| 9 | FAM163B | family with sequence similarity 163 member B | 196990 | 197859 | 123454 | 32, 8, |
| 9 | FAM166A | family with sequence similarity 166 member A | 188163 | 188229 | 197768 | 38, 27, 25 |
| 9 | FAM166B | family with sequence similarity 166 member B | 215187 | 198853 | 107140 | 2, 35, 20 |
| 9 | FAM189A2 | family with sequence similarity 189 member A2 | 135063 | 283143 | 107282 | 18, 40, 39 |
| 9 | FAM205A | family with sequence similarity 205 member A | 205108 | 137077 | 187791 | 38, |
| 9 | FAM205C | family with sequence similarity 205 member C | 187791 | 205108 | 122733 | |
| 9 | FAM206A | family with sequence similarity 206 member A | 119328 | 070061 | 119326 | 29, 31, 15 |
| 9 | FAM214B | family with sequence similarity 214 member B | 005238 | 165283 | 198722 | 45, 39, 10 |
| 9 | FAM219A | family with sequence similarity 219 member A | 164970 | 164972 | 122735 | 26, 32, 35 |
| 9 | FAM221B | family with sequence similarity 221 member B | 204930 | 137103 | 168828 | 38, 37, 32 |
| 9 | FAM69B | family with sequence similarity 69 member B | 165716 | 169692 | 187922 | 15, 29, 20 |
| 9 | FAM78A | family with sequence similarity 78 member A | 126882 | 126883 | 160539 | 7, 22, 43 |
| 9 | FANCC | Fanconi anemia complementation group C | 158169 | 148120 | 185920 | 38, 17, 3 |
| 9 | FANCG | Fanconi anemia complementation group G | 221829 | 165280 | 165282 | 9, 21, 39 |
| 9 | FUBP3 | far upstream element binding protein 3 | 107164 | 130707 | 130711 | 34, 40, 12 |
| 9 | FBXW2 | F-box and WD repeat domain containing 2 | 119402 | 106780 | 095261 | 40, 22, 15 |
| 9 | FBXW5 | F-box and WD repeat domain containing 5 | 159069 | 127191 | 176919 | 38, 9, 34 |
| 9 | FBXO10 | F-box protein 10 | 147912 | 137054 | 256966 | 22, 41, 36 |
| 9 | FRMPD1 | FERM and PDZ domain containing 1 | 070601 | 255872 | 165275 | 14, 2, 38 |
| 9 | FRMD3 | FERM domain containing 3 | 172159 | 165105 | 148057 | |
| 9 | FRRS1L | ferric chelate reductase 1 like | 260230 | 106771 | 095203 | 38, 28, 27 |
| 9 | FIBCD1 | fibrinogen C domain containing 1 | 130720 | 188710 | 050555 | 9, 2, 29 |
| 9 | FSD1L | fibronectin type III and SPRY domain containing 1 like | 106701 | 070214 | 106692 | 1, 26, 21 |
| 9 | FCN1 | ficolin 1 | 085265 | 160339 | 130558 | 5, 7, 20 |
| 9 | FCN2 | ficolin 2 | 160339 | 130635 | 085265 | 2, 19, 5 |
| 9 | FKBP15 | FK506 binding protein 15 | 119321 | 136867 | 136868 | 24, 36, 43 |
| 9 | FOCAD | focadhesin | 188352 | 171843 | 188921 | |
| 9 | FPGS | folylpolyglutamate synthase | 136877 | 136807 | 106991 | 21, 9, 22 |
| 9 | FOXB2 | forkhead box B2 | 204612 | 106772 | 197969 | |
| 9 | FOXD4 | forkhead box D4 | 170122 | 181404 | 172785 | 33, 20, 44 |
| 9 | FOXD4L3 | forkhead box D4-like 3 | 187559 | 196873 | 154330 | |
| 9 | FOXD4L4 | forkhead box D4-like 4 | 184659 | 147996 | 276203 | 38, |
| 9 | FOXD4L5 | forkhead box D4-like 5 | 204779 | 172014 | 147996 | |
| 9 | FOXD4L6 | forkhead box D4-like 6 | 273514 | 183148 | 215126 | 16, |
| 9 | FOXE1 | forkhead box E1 | 178919 | 136936 | 136932 | 40, 35, 38 |
| 9 | FNBP1 | formin binding protein 1 | 187239 | 136878 | 148358 | 22, 1, 41 |
| 9 | FREM1 | FRAS1 related extracellular matrix 1 | 164946 | 147869 | 155158 | 11, 26, 17 |
| 9 | FXN | frataxin | 165060 | 165059 | 119139 | 29, 27, 37 |
| 9 | FBP1 | fructose-bisphosphatase 1 | 165140 | 130957 | 148120 | 37, 20, 17 |
| 9 | FBP2 | fructose-bisphosphatase 2 | 130957 | 148110 | 165140 | 29, 25, 15 |
| 9 | FUT7 | fucosyltransferase 7 | 180549 | 107331 | 279073 | 1, 22, 3 |
| 9 | FKTN | fukutin | 106692 | 106701 | 186051 | 29, 43, 4 |
| 9 | FGD3 | FYVE, RhoGEF and PH domain containing 3 | 127084 | 127081 | 157303 | 39, 5, 41 |
| 9 | GKAP1 | G kinase anchoring protein 1 | 165113 | 135018 | 165115 | 38, 29, 10 |
| 9 | GNA14 | G protein subunit alpha 14 | 156049 | 197969 | 156052 | 45, 40, 44 |
| 9 | GNAQ | G protein subunit alpha q | 156052 | 156049 | 148019 | 35, 32, 4 |
| 9 | GNG10 | G protein subunit gamma 10 | 242616 | 244115 | 165181 | |
| 9 | GPR107 | G protein-coupled receptor 107 | 148358 | 187239 | 107130 | |
| 9 | GPR21 | G protein-coupled receptor 21 | 188394 | 011454 | 165209 | 42, 25, 8 |
| 9 | GALT | galactose-1-phosphate uridylyltransferase | 213930 | 147955 | 258728 | 39, 10, 38 |
| 9 | GABBR2 | gamma-aminobutyric acid type B receptor subunit 2 | 136928 | 095383 | 165138 | 2, 8, 45 |
| 9 | GSN | gelsolin | 148180 | 119396 | 283299 | 1, 24, 3 |
| 9 | GTF3C4 | general transcription factor IIIC subunit 4 | 125484 | 125485 | 165695 | 6, 45, 38 |
| 9 | GTF3C5 | general transcription factor IIIC subunit 5 | 148308 | 165702 | 170835 | 39, 3, 36 |
| 9 | GLE1 | GLE1, RNA export mediator | 119392 | 136811 | 197694 | 38, 41, 39 |
| 9 | GLIPR2 | GLI pathogenesis related 2 | 122694 | 122707 | 185972 | 43, 5, 1 |
| 9 | GLIS3 | GLIS family zinc finger 3 | 107249 | 279745 | 106688 | |
| 9 | GBGT1 | globoside alpha-1,3-N-acetylgalactosaminyltransferase 1 | 148288 | 160271 | 171102 | 7, 1, 2 |
| 9 | GNE | glucosamine (UDP-N-acetyl)-2-epimerase/N-acetylmannosamine kinase | 159921 | 122705 | 137075 | 42, 24, 29 |
| 9 | GCNT1 | glucosaminyl (N-acetyl) transferase 1, core 2 | 187210 | 135002 | 106772 | 29, 6, 37 |
| 9 | GBA2 | glucosylceramidase beta 2 | 070610 | 107175 | 107185 | 43, 30, 18 |
| 9 | GRIN1 | glutamate ionotropic receptor NMDA type subunit 1 | 176884 | 176978 | 184709 | |
| 9 | GRIN3A | glutamate ionotropic receptor NMDA type subunit 3A | 198785 | 155827 | 188386 | 26, 2, 9 |
| 9 | GLDC | glycine decarboxylase | 178445 | 147854 | 274527 | 17, 19, 12 |
| 9 | GLT6D1 | glycosyltransferase 6 domain containing 1 | 204007 | 236543 | 148386 | 38, |
| 9 | GRHPR | glyoxylate and hydroxypyruvate reductase | 137106 | 147905 | 168795 | 21, 3, 26 |
| 9 | GOLM1 | golgi membrane protein 1 | 135052 | 135040 | 187753 | 42, 3, 1 |
| 9 | GOLGA1 | golgin A1 | 136935 | 136950 | 173611 | 26, 30, 31 |
| 9 | GOLGA2 | golgin A2 | 167110 | 106976 | 175854 | 29, 19, 28 |
| 9 | GPSM1 | G-protein signaling modulator 1 | 160360 | 260220 | 213221 | 26, 14, 6 |
| 9 | GADD45G | growth arrest and DNA damage inducible gamma | 130222 | 187764 | 165023 | 28, 29, 32 |
| 9 | GAS1 | growth arrest specific 1 | 180447 | 083223 | 196730 | 15, 6, 45 |
| 9 | GFI1B | growth factor independent 1B transcriptional repressor | 165702 | 165699 | 148308 | 36, 7, 19 |
| 9 | GAPVD1 | GTPase activating protein and VPS9 domains 1 | 165219 | 044574 | 119487 | 24, 11, 40 |
| 9 | GARNL3 | GTPase activating Rap/RanGAP domain like 3 | 136895 | 136859 | 136856 | 11, 26, 8 |
| 9 | GDA | guanine deaminase | 119125 | 204669 | 107372 | 9, 8, 32 |
| 9 | GVQW1 | GVQW motif containing 1 | 241043 | 165264 | 122728 | 17, 33, 38 |
| 9 | HDHD3 | haloacid dehalogenase like hydrolase domain containing 3 | 119431 | 119411 | 148218 | 29, 16, 27 |
| 9 | HAUS6 | HAUS augmin like complex subunit 6 | 147874 | 155876 | 147872 | 12, 25, 38 |
| 9 | HSPA5 | heat shock protein family A (Hsp70) member 5 | 044574 | 136933 | 165219 | 40, 13, 1 |
| 9 | HMCN2 | hemicentin 2 | 148357 | 107130 | 130707 | 39, 16, 9 |
| 9 | HEMGN | hemogen | 136929 | 136932 | 136938 | 7, 38, 36 |
| 9 | HNRNPK | heterogeneous nuclear ribonucleoprotein K | 165119 | 165118 | 178966 | 39, 30, 18 |
| 9 | HRCT1 | histidine rich carboxyl terminus 1 | 196196 | 168828 | 235387 | 6, 2, 32 |
| 9 | HINT2 | histidine triad nucleotide binding protein 2 | 137133 | 137098 | 137103 | 25, 41, 31 |
| 9 | HABP4 | hyaluronan binding protein 4 | 130956 | 165244 | 081377 | 14, 40, 23 |
| 9 | HSD17B3 | hydroxysteroid 17-beta dehydrogenase 3 | 130948 | 182150 | 130958 | 38, 6, 8 |
| 9 | HSDL2 | hydroxysteroid dehydrogenase like 2 | 119471 | 119314 | 165185 | 15, 17, 26 |
| 9 | IDNK | IDNK, gluconokinase | 148057 | 172159 | 135018 | 10, 28, 15 |
| 9 | IER5L | immediate early response 5 like | 188483 | 119383 | 148335 | 37, 3, 22 |
| 9 | IKBKAP | inhibitor of kappa light polypeptide gene enhancer in B-cells, kinase complex- | 070061 | 187003 | 119328 | 2, 42, 29 |
| | | associated protein | | | | |
| 9 | INPP5E | inositol polyphosphate-5-phosphatase E | 148384 | 165688 | 148396 | 26, 12, 7 |
| 9 | IPPK | inositol-pentakisphosphate 2-kinase | 127080 | 106823 | 185963 | 33, 24, 22 |
| 9 | INSL4 | insulin like 4 | 120211 | 120210 | 107014 | 32, 10, |
| 9 | INSL6 | insulin like 6 | 120210 | 096968 | 120211 | 38, |
| 9 | IGFBPL1 | insulin like growth factor binding protein like 1 | 137142 | 137124 | 283486 | 26, 31, 14 |
| 9 | IFNA1 | interferon alpha 1 | 197919 | 120242 | 184995 | |
| 9 | IFNA10 | interferon alpha 10 | 186803 | 214042 | 147885 | |
| 9 | IFNA13 | interferon alpha 13 | 233816 | 120235 | 188379 | |
| 9 | IFNA14 | interferon alpha 14 | 228083 | 234829 | 147873 | |
| 9 | IFNA16 | interferon alpha 16 | 147885 | 186803 | 234829 | |
| 9 | IFNA17 | interferon alpha 17 | 234829 | 147885 | 228083 | |
| 9 | IFNA2 | interferon alpha 2 | 188379 | 233816 | 120242 | |
| 9 | IFNA21 | interferon alpha 21 | 137080 | 177047 | 236637 | |
| 9 | IFNA4 | interferon alpha 4 | 236637 | 137080 | 214042 | |
| 9 | IFNA5 | interferon alpha 5 | 147873 | 228083 | 198642 | |
| 9 | IFNA6 | interferon alpha 6 | 120235 | 198642 | 233816 | |
| 9 | IFNA7 | interferon alpha 7 | 214042 | 236637 | 186803 | |
| 9 | IFNA8 | interferon alpha 8 | 120242 | 188379 | 197919 | |
| 9 | IFNB1 | interferon beta 1 | 171855 | 188921 | 177047 | |
| 9 | IFNE | interferon epsilon | 184995 | 197919 | 099810 | 10, |
| 9 | IFNK | interferon kappa | 147896 | 120162 | 147894 | 44, |
| 9 | IFNW1 | interferon omega 1 | 177047 | 171855 | 137080 | 20, |
| 9 | IL11RA | interleukin 11 receptor subunit alpha | 137070 | 258728 | 213927 | 29, 1, 36 |
| 9 | IL33 | interleukin 33 | 137033 | 137040 | 170777 | 2, 45, 42 |
| 9 | IFT74 | intraflagellar transport 74 | 096872 | 137055 | 184434 | 4, 11, 7 |
| 9 | INIP | INTS3 and NABP interacting protein | 148153 | 165185 | 148158 | 30, 43, 23 |
| 9 | INVS | inversin | 119509 | 023318 | 136891 | 43, 29, 39 |
| 9 | ISCA1 | iron-sulfur cluster assembly 1 | 135070 | 187753 | 083223 | 15, 13, 9 |
| 9 | IARS | isoleucyl-tRNA synthetase | 196305 | 090054 | 279007 | 3, 40, 37 |
| 9 | IZUMO3 | IZUMO family member 3 | 205442 | 107105 | 198680 | 38, |
| 9 | JAK2 | Janus kinase 2 | 096968 | 120158 | 120210 | 7, 45, 1 |
| 9 | KLHL9 | kelch like family member 9 | 198642 | 147873 | 120235 | |
| 9 | KIAA0368 | KIAA0368 | 136813 | 171133 | 173258 | 37, 1, 23 |
| 9 | KIAA1161 | KIAA1161 | 164976 | 164978 | 164972 | 17, 32, 16 |
| 9 | KIAA1958 | KIAA1958 | 165185 | 119471 | 148153 | 18, 44, 12 |
| 9 | KIAA2026 | KIAA2026 | 183354 | 099219 | 120215 | 37, 6, 32 |
| 9 | KIF12 | kinesin family member 12 | 136883 | 106927 | 196739 | 17, 3, 28 |
| 9 | KIF24 | kinesin family member 24 | 186638 | 165006 | 164978 | 22, 5, 27 |
| 9 | KIF27 | kinesin family member 27 | 165115 | 165113 | 165118 | 30, 7, 6 |
| 9 | KANK1 | KN motif and ankyrin repeat domains 1 | 107104 | 107099 | 137090 | 1, 43, 40 |
| 9 | KLF4 | Kruppel like factor 4 | 136826 | 119318 | 148156 | 1, 18, 11 |
| 9 | KLF9 | Kruppel like factor 9 | 119138 | 198887 | 083067 | 26, 17, 25 |
| 9 | KYAT1 | kynurenine aminotransferase 1 | 171097 | 198917 | 136802 | 9, 37, 30 |
| 9 | LAMC3 | laminin subunit gamma 3 | 050555 | 130720 | 126878 | 32, 38, 24 |
| 9 | LURAP1L | leucine rich adaptor protein 1 like | 153714 | 107165 | 107186 | 26, 36, 45 |
| 9 | LINGO2 | leucine rich repeat and Ig domain containing 2 | 174482 | 147894 | 122729 | 11, 38, 23 |
| 9 | LRSAM1 | leucine rich repeat and sterile alpha motif containing 1 | 148356 | 197958 | 136830 | 9, 22, 8 |
| 9 | LRRC19 | leucine rich repeat containing 19 | 184434 | 096872 | 120156 | 17, 37, 25 |
| 9 | LRRC26 | leucine rich repeat containing 26 | 184709 | 176884 | 261793 | 3, 16, 33 |
| 9 | LRRC8A | leucine rich repeat containing 8 family member A | 136802 | 171097 | 175287 | 24, 40, 6 |
| 9 | LHX2 | LIM homeobox 2 | 106689 | 119522 | 119408 | 26, 14, 8 |
| 9 | LHX3 | LIM homeobox 3 | 107187 | 238227 | 165661 | 14, 8, |
| 9 | LHX6 | LIM homeobox 6 | 106852 | 185681 | 119446 | 8, 15, 33 |
| 9 | LMX1B | LIM homeobox transcription factor 1 beta | 136944 | 196814 | 169155 | 23, 14, 20 |
| 9 | LCN1 | lipocalin 1 | 160349 | 122140 | 122136 | 14, 38, |
| 9 | LCN10 | lipocalin 10 | 187922 | 165716 | 267206 | 36, 5, 15 |
| 9 | LCN12 | lipocalin 12 | 184925 | 176919 | 107317 | 36, 20, 17 |
| 9 | LCN15 | lipocalin 15 | 177984 | 204001 | 244187 | 16, |
| 9 | LCN2 | lipocalin 2 | 148346 | 148334 | 171159 | 37, 5, 30 |
| 9 | LCN6 | lipocalin 6 | 267206 | 187922 | 204001 | 44, 39, 38 |
| 9 | LCN8 | lipocalin 8 | 204001 | 267206 | 177984 | 36, 22, 7 |
| 9 | LCN9 | lipocalin 9 | 148386 | 204007 | 165643 | 12, |
| 9 | LCNL1 | lipocalin like 1 | 214402 | 107317 | 148362 | 26, 8, 38 |
| 9 | LINC00094 | Long intergenic non-protein coding RNA 94 | 235106 | 160293 | 169925 | 34, 29, 14 |
| 9 | LINC00961 | Long intergenic non-protein coding RNA 961 | 235387 | 196196 | 278889 | |
| 9 | KDM4C | lysine demethylase 4C | 107077 | 282960 | 137038 | 10, 42, 1 |
| 9 | LPAR1 | lysophosphatidic acid receptor 1 | 198121 | 030304 | 171133 | 11, 10, 8 |
| 9 | MFSD14B | major facilitator superfamily domain containing 14B | 148110 | 130950 | 130957 | 29, 34, 22 |
| 9 | MFSD14C | Major facilitator superfamily domain containing 14C | 196312 | 196597 | 188152 | |
| 9 | MAMDC2 | MAM domain containing 2 | 165072 | 204711 | 198887 | 12, 31, 43 |
| 9 | MAMDC4 | MAM domain containing 4 | 177943 | 054148 | 107223 | 19, 28, 44 |
| 9 | MAN1B1 | mannosidase alpha class 1B member 1 | 177239 | 197355 | 176978 | 9, 45, 36 |
| 9 | MELK | maternal embryonic leucine zipper kinase | 165304 | 137075 | 196092 | 13, 3, 24 |
| 9 | MED22 | mediator complex subunit 22 | 148297 | 148296 | 148303 | 42, 39, 3 |
| 9 | MED27 | mediator complex subunit 27 | 160563 | 107263 | 196358 | 13, 30, 10 |
| 9 | MLANA | melan-A | 120215 | 183354 | 137040 | 35, 11, 14 |
| 9 | MTAP | methylthioadenosine phosphorylase | 099810 | 184995 | 264545 | 1, 12, 33 |
| 9 | MSMP | microseminoprotein, prostate associated | 215183 | 107185 | 159899 | 40, 7, 10 |
| 9 | MRPL41 | mitochondrial ribosomal protein L41 | 182154 | 130653 | 148399 | 27, 23, 3 |
| 9 | MRPL50 | mitochondrial ribosomal protein L50 | 136897 | 136881 | 136870 | 4, 29, 34 |
| 9 | MRPS2 | mitochondrial ribosomal protein S2 | 122140 | 160345 | 160349 | 21, 9, 29 |
| 9 | MRRF | mitochondrial ribosome recycling factor | 148187 | 119446 | 095303 | 9, 34, 43 |
| 9 | MAPKAP1 | mitogen-activated protein kinase associated protein 1 | 119487 | 165219 | 167081 | 9, 14, 40 |
| 9 | MIGA2 | mitoguardin 2 | 148343 | 148341 | 167130 | 7, 24, 39 |
| 9 | MLLT3 | MLLT3, super elongation complex subunit | 171843 | 155886 | 188352 | 6, 11, 8 |
| 9 | MOB3B | MOB kinase activator 3B | 120162 | 120160 | 147896 | 32, 31, 17 |
| 9 | MORN5 | MORN repeat containing 5 | 185681 | 119421 | 106852 | 30, 38, 44 |
| 9 | MSANTD3- | MSANTD3-TMEFF1 readthrough | 251349 | 066697 | 241697 | |
| | TMEFF1 | | | | | |
| 9 | MEGF9 | multiple EGF like domains 9 | 106780 | 136861 | 119402 | 11, 26, 14 |
| 9 | MPDZ | multiple PDZ domain crumbs cell polarity complex component | 107186 | 153714 | 147862 | 26, 1, 43 |
| 9 | MVB12B | multivesicular body subunit 12B | 196814 | 167081 | 136944 | 26, 14, 8 |
| 9 | MUSK | muscle associated receptor tyrosine kinase | 030304 | 165124 | 198121 | 24, 25, 38 |
| 9 | MURO | muscle related coiled-coil protein | 170681 | 241697 | 148123 | 15, 25, 24 |
| 9 | MSANTD3 | Myb/SANT DNA binding domain containing 3 | 066697 | 136891 | 251349 | 6, 9, 24 |
| 9 | NAA35 | N(alpha)-acetyltransferase 35, NatC auxiliary subunit | 135040 | 135049 | 135052 | 18, 31, 29 |
| 9 | NACC2 | NACC family member 2 | 148411 | 130560 | 238227 | 34, 9, 15 |
| 9 | NANS | N-acetylneuraminate synthase | 095380 | 136938 | 106785 | 9, 6, 37 |
| 9 | NDUFA8 | NADH: ubiquinone oxidoreductase subunit A8 | 119421 | 175764 | 185681 | 28, 29, 15 |
| 9 | NDUFB6 | NADH: ubiquinone oxidoreductase subunit B6 | 165264 | 235453 | 241043 | 29, 45, 11 |
| 9 | NDOR1 | NADPH dependent diflavin oxidoreductase 1 | 188566 | 187713 | 283590 | 2, 4, 37 |
| 9 | NDOR1 | NADPH dependent diflavin oxidoreductase 1 | 283590 | 188566 | 212864 | 2, 4, 37 |
| 9 | NOXA1 | NADPH oxidase activator 1 | 188747 | 187609 | 188833 | 21, 23, 22 |
| 9 | NPR2 | natriuretic peptide receptor 2 | 159899 | 215183 | 137098 | 31, 43, 2 |
| 9 | NELFB | negative elongation factor complex member B | 188986 | 197768 | 198113 | 3, 9, 26 |
| 9 | NTNG2 | netrin G2 | 196358 | 160563 | 107290 | 14, 2, 5 |
| 9 | NPDC1 | neural proliferation, differentiation and control 1 | 107281 | 279073 | 054179 | 26, 31, 9 |
| 9 | NCS1 | neuronal calcium sensor 1 | 107130 | 148358 | 148357 | 26, 27, 8 |
| 9 | NTRK2 | neurotrophic receptor tyrosine kinase 2 | 148053 | 197506 | 135049 | 24, 13, 8 |
| 9 | NMRK1 | nicotinamide riboside kinase 1 | 106733 | 156017 | 134996 | 29, 9, 23 |
| 9 | NEK6 | NIMA related kinase 6 | 119408 | 106689 | 136930 | 41, 43, 3 |
| 9 | NINJ1 | ninjurin 1 | 131669 | 165233 | 165238 | 1, 34, 33 |
| 9 | NIPSNAP3A | nipsnap homolog 3A | 136783 | 179055 | 165028 | 7, 2, 45 |
| 9 | NIPSNAP3B | nipsnap homolog 3B | 165028 | 136783 | 165029 | 1, 25, 7 |
| 9 | NSMF | NMDA receptor synaptonuclear signaling and neuronal migration factor | 165802 | 188833 | 130653 | 34, 26, 3 |
| 9 | NOTCH1 | notch 1 | 148400 | 148396 | 172889 | 22, 1, 15 |
| 9 | NRARP | NOTCH-regulated ankyrin repeat protein | 198435 | 198113 | 187609 | 3, 28, 14 |
| 9 | NTMT1 | N-terminal Xaa-Pro-Lys N-methyltransferase 1 | 148335 | 188483 | 179058 | 34, 37, 35 |
| 9 | NAIF1 | nuclear apoptosis inducing factor 1 | 171169 | 167106 | 148339 | 36, 13, 24 |
| 9 | NCBP1 | nuclear cap binding protein subunit 1 | 136937 | 136925 | 136936 | 24, 39, 41 |
| 9 | NFIB | nuclear factor I B | 147862 | 107186 | 175893 | 11, 44, 40 |
| 9 | NFIL3 | nuclear factor, interleukin 3 regulated | 165030 | 148090 | 169071 | 31, 17, 1 |
| 9 | NR4A3 | nuclear receptor subfamily 4 group A member 3 | 119508 | 106803 | 136874 | 1, 5, 26 |
| 9 | NR5A1 | nuclear receptor subfamily 5 group A member 1 | 136931 | 180264 | 148200 | 36, 2, 38 |
| 9 | NR6A1 | nuclear receptor subfamily 6 group A member 1 | 148200 | 136931 | 185585 | 27, 38, 5 |
| 9 | NFX1 | nuclear transcription factor, X-box binding 1 | 086102 | 086065 | 165269 | 24, 42, 18 |
| 9 | NOL6 | nucleolar protein 6 | 165271 | 165272 | 230453 | 43, 24, 27 |
| 9 | NOL8 | nucleolar protein 8 | 198000 | 279007 | 188312 | 22, 24, 29 |
| 9 | NUP188 | nucleoporin 188 | 095319 | 175283 | 148341 | 21, 41, 44 |
| 9 | NUP214 | nucleoporin 214 | 126883 | 126878 | 126882 | |
| 9 | NXNL2 | nucleoredoxin-like 2 | 130045 | 106723 | 186354 | 23, 27, 17 |
| 9 | NUDT2 | nudix hydrolase 2 | 164978 | 186638 | 164976 | 37, 41, 28 |
| 9 | NUTM2F | NUT family member 2F | 130950 | 175787 | 148110 | 38, |
| 9 | NUTM2G | NUT family member 2G | 188152 | 196312 | 136943 | |
| 9 | OBP2A | odorant binding protein 2A | 122136 | 160349 | 122133 | |
| 9 | OBP2B | odorant binding protein 2B | 171102 | 148288 | 175164 | 38, 44, |
| 9 | OLFM1 | olfactomedin 1 | 130558 | 085265 | 196422 | 26, 11, 31 |
| 9 | OLFML2A | olfactomedin like 2A | 185585 | 148200 | 136918 | 11, 15, 27 |
| 9 | OR1B1 | olfactory receptor family 1 subfamily B member 1 (gene/pseudogene) | 280094 | 165202 | 173679 | |
| 9 | OR1J1 | olfactory receptor family 1 subfamily J member 1 | 136834 | 095303 | 197233 | |
| 9 | OR1J2 | olfactory receptor family 1 subfamily J member 2 | 197233 | 136834 | 239590 | |
| 9 | OR1J4 | olfactory receptor family 1 subfamily J member 4 | 239590 | 197233 | 171505 | |
| 9 | OR1K1 | olfactory receptor family 1 subfamily K member 1 | 165204 | 136940 | 056586 | |
| 9 | OR1L1 | olfactory receptor family 1 subfamily L member 1 | 173679 | 280094 | 171481 | |
| 9 | OR1L3 | olfactory receptor family 1 subfamily L member 3 | 171481 | 173679 | 136939 | |
| 9 | OR1L4 | olfactory receptor family 1 subfamily L member 4 | 136939 | 171481 | 171459 | |
| 9 | OR1L6 | olfactory receptor family 1 subfamily L member 6 | 171459 | 136939 | 148215 | |
| 9 | OR1L8 | olfactory receptor family 1 subfamily L member 8 | 171496 | 171501 | 165202 | |
| 9 | OR1N1 | olfactory receptor family 1 subfamily N member 1 | 171505 | 239590 | 171501 | |
| 9 | OR1N2 | olfactory receptor family 1 subfamily N member 2 | 171501 | 171505 | 171496 | |
| 9 | OR1Q1 | olfactory receptor family 1 subfamily Q member 1 | 165202 | 171496 | 280094 | |
| 9 | OR13C2 | olfactory receptor family 13 subfamily C member 2 | 276119 | 277556 | 136839 | |
| 9 | OR13C3 | olfactory receptor family 13 subfamily C member 3 | 204246 | 148136 | 186943 | |
| 9 | OR13C4 | olfactory receptor family 13 subfamily C member 4 | 148136 | 186881 | 204246 | |
| 9 | OR13C5 | olfactory receptor family 13 subfamily C member 5 | 277556 | 186943 | 276119 | |
| 9 | OR13C7 | olfactory receptor family 13 subfamily C member 7 (gene/pseudogene) | 243641 | 278889 | 122707 | |
| 9 | OR13C8 | olfactory receptor family 13 subfamily C member 8 | 186943 | 204246 | 277556 | |
| 9 | OR13C9 | olfactory receptor family 13 subfamily C member 9 | 136839 | 276119 | 179055 | |
| 9 | OR13D1 | olfactory receptor family 13 subfamily D member 1 | 179055 | 136839 | 136783 | |
| 9 | OR13F1 | olfactory receptor family 13 subfamily F member 1 | 186881 | 136824 | 148136 | |
| 9 | OR13J1 | olfactory receptor family 13 subfamily J member 1 | 168828 | 204930 | 196196 | |
| 9 | OR2K2 | olfactory receptor family 2 subfamily K member 2 | 171133 | 198121 | 136813 | |
| 9 | OR2S2 | olfactory receptor family 2 subfamily S member 2 (gene/pseudogene) | 278889 | 235387 | 243641 | |
| 9 | OR5C1 | olfactory receptor family 5 subfamily C member 1 | 148215 | 171459 | 136940 | |
| 9 | ORM1 | orosomucoid 1 | 229314 | 196739 | 228278 | 19, 33, 25 |
| 9 | ORM2 | orosomucoid 2 | 228278 | 229314 | 106948 | 19, 33, 37 |
| 9 | OSTF1 | osteoclast stimulating factor 1 | 134996 | 106733 | 099139 | 41, 22, 44 |
| 9 | OGN | osteoglycin | 106809 | 188312 | 127083 | 11, 45, 44 |
| 9 | OMD | osteomodulin | 127083 | 106809 | 106819 | 42, 15, 26 |
| 9 | ODF2 | outer dense fiber of sperm tails 2 | 136811 | 167123 | 119392 | 38, 43, 7 |
| 9 | PAX5 | paired box 5 | 196092 | 165304 | 147905 | 22, 41, 36 |
| 9 | PRRX2 | paired related homeobox 2 | 167157 | 148331 | 148344 | 33, 6, 44 |
| 9 | PALM2-AKAP2 | PALM2-AKAP2 readthrough | 157654 | 243444 | 241978 | |
| 9 | PAPPA | pappalysin 1 | 182752 | 173077 | 148219 | 32, 45, 26 |
| 9 | PALM2 | paralemmin 2 | 243444 | 070159 | 157654 | |
| 9 | PNPLA7 | patatin like phospholipase domain containing 7 | 130653 | 165802 | 182154 | 42, 22, 37 |
| 9 | PTCH1 | patched 1 | 185920 | 158169 | 182150 | 11, 38, 13 |
| 9 | PBX3 | PBX homeobox 3 | 167081 | 119487 | 196814 | |
| 9 | PSIP1 | PC4 and SFRS1 interacting protein 1 | 164985 | 164975 | 164989 | 11, 7, 29 |
| 9 | PMPCA | peptidase, mitochondrial processing alpha subunit | 165688 | 165689 | 148384 | 21, 2, 36 |
| 9 | PTRH1 | peptidyl-tRNA hydrolase 1 homolog | 187024 | 136854 | 160401 | 9, 3, 27 |
| 9 | PLIN2 | perilipin 2 | 147872 | 147874 | 137145 | 1, 11, 19 |
| 9 | PHF19 | PHD finger protein 19 | 119403 | 095261 | 056558 | 43, 6, 3 |
| 9 | PHF2 | PHD finger protein 2 | 197724 | 048828 | 131668 | 26, 6, 14 |
| 9 | PHF24 | PHD finger protein 24 | 122733 | 187791 | 137094 | |
| 9 | PDCL | phosducin like | 136940 | 148215 | 165204 | 26, 45, 6 |
| 9 | PIGO | phosphatidylinositol glycan anchor biosynthesis class O | 165282 | 221829 | 165283 | 40, 3, 26 |
| 9 | PIP5KL1 | phosphatidylinositol-4-phosphate 5-kinase like 1 | 167103 | 136840 | 136908 | 10, |
| 9 | PIP5K1B | phosphatidylinositol-4-phosphate 5-kinase type 1 beta | 107242 | 181778 | 187866 | 11, 38, 16 |
| 9 | PGM5 | phosphoglucomutase 5 | 154330 | 187559 | 181778 | 1, 44, 42 |
| 9 | PHPT1 | phosphohistidine phosphatase 1 | 054148 | 232434 | 177943 | 29, 31, 15 |
| 9 | PLAA | phospholipase A2 activating protein | 137055 | 120159 | 096872 | 9, 1, 3 |
| 9 | PLPP6 | phospholipid phosphatase 6 | 205808 | 106686 | 106993 | 4, 19, 39 |
| 9 | PLPP7 | phospholipid phosphatase 7 (inactive) | 160539 | 126882 | 130723 | 15, 25, 24 |
| 9 | PLPPR1 | phospholipid phosphatase related 1 | 148123 | 170681 | 136881 | 8, 26, 17 |
| 9 | PSAT1 | phosphoserine aminotransferase 1 | 135069 | 148019 | 106829 | 21, 9, 3 |
| 9 | PHYHD1 | phytanoyl-CoA dioxygenase domain containing 1 | 175287 | 136802 | 251184 | 29, 26, 30 |
| 9 | PLGRKT | plasminogen receptor with a C-terminal lysine | 107020 | 107018 | 120217 | 10, 26, 2 |
| 9 | GALNT12 | polypeptide N-acetylgalactosaminyltransferase 12 | 119514 | 165138 | 204291 | 34, 16, 31 |
| 9 | PTBP3 | polypyrimidine tract binding protein 3 | 119314 | 106868 | 119471 | 26, 11, 22 |
| 9 | KCNT1 | potassium sodium-activated channel subfamily T member 1 | 107147 | 165643 | 130559 | 26, 8, 38 |
| 9 | KCNV2 | potassium voltage-gated channel modifier subfamily V member 2 | 168263 | 147852 | 080608 | 14, 10, 19 |
| 9 | PRDM12 | PR/SET domain 12 | 130711 | 107164 | 130713 | 25, |
| 9 | PRPF4 | pre-mRNA processing factor 4 | 136875 | 176386 | 165188 | 13, 40, 39 |
| 9 | PAEP | progestagen associated endometrial protein | 122133 | 122136 | 236543 | 44, 27, 33 |
| 9 | PDCD1LG2 | programmed cell death 1 ligand 2 | 197646 | 120217 | 107036 | 40, 15, 44 |
| 9 | PRRC2B | proline rich coiled-coil 2B | 130723 | 160539 | 130714 | 18, 43, 40 |
| 9 | PCSK5 | proprotein convertase subtilisin/kexin type 5 | 099139 | 134996 | 135002 | 40, 39, 13 |
| 9 | PTGDS | prostaglandin D2 synthase | 107317 | 184925 | 214402 | 14, 26, 38 |
| 9 | PTGES | prostaglandin E synthase | 148344 | 167157 | 136816 | 33, 32, 20 |
| 9 | PTGES2 | prostaglandin E synthase 2 | 148334 | 148339 | 148346 | 9, 41, 40 |
| 9 | PTGR1 | prostaglandin reductase 1 | 106853 | 283263 | 059769 | 19, 1, 17 |
| 9 | PTGS1 | prostaglandin-endoperoxide synthase 1 | 095303 | 148187 | 136834 | 45, 24, 35 |
| 9 | PRSS3 | protease, serine 3 | 010438 | 229063 | 107341 | 28, 18, 3 |
| 9 | PSMD5 | proteasome 26S subunit, non-ATPase 5 | 095261 | 119402 | 119403 | 45, 38, 40 |
| 9 | PSMB7 | proteasome subunit beta 7 | 136930 | 119408 | 180264 | 43, 3, 30 |
| 9 | PRKACG | protein kinase cAMP-activated catalytic subunit gamma | 165059 | 187866 | 165060 | 38, 27, 8 |
| 9 | PKN3 | protein kinase N3 | 160447 | 119335 | 160446 | 1, 29, 18 |
| 9 | POMT1 | protein O-mannosyltransferase 1 | 130714 | 130723 | 130717 | 39, 38, 43 |
| 9 | PPP1R26 | protein phosphatase 1 regulatory subunit 26 | 196422 | 130558 | 160345 | 9, 3, 17 |
| 9 | PTPA | protein phosphatase 2 phosphatase activator | 119383 | 095321 | 188483 | 9, 3, 24 |
| 9 | PPP3R2 | protein phosphatase 3 regulatory subunit B, beta | 188386 | 198785 | 155833 | 38, 8, |
| 9 | PPP6C | protein phosphatase 6 catalytic subunit | 119414 | 173611 | 136933 | 4, 38, 29 |
| 9 | PTAR1 | protein prenyltransferase alpha subunit repeat containing 1 | 188647 | 107282 | 204711 | 39, 11, 42 |
| 9 | PTPDC1 | protein tyrosine phosphatase domain containing 1 | 158079 | 131668 | 175787 | 8, 6, 38 |
| 9 | PTPN3 | protein tyrosine phosphatase, non-receptor type 3 | 070159 | 095203 | 243444 | |
| 9 | PTPRD | protein tyrosine phosphatase, receptor type D | 153707 | 137038 | 107165 | 29, 18, 11 |
| 9 | PRUNE2 | prune homolog 2 | 106772 | 187210 | 204612 | 31, 26, 8 |
| 9 | PUM3 | pumilio RNA binding family member 3 | 080608 | 168263 | 080298 | 13, 11, 45 |
| 9 | QRFP | pyroglutamylated RFamide peptide | 188710 | 097007 | 130720 | 14, 38, |
| 9 | QSOX2 | quiescin sulfhydryl oxidase 2 | 165661 | 107187 | 279813 | 11, 22, 36 |
| 9 | RABGAP1 | RAB GTPase activating protein 1 | 011454 | 171448 | 188394 | |
| 9 | RABL6 | RAB, member RAS oncogene family-like 6 | 196642 | 213213 | 232434 | 21, 9, 12 |
| 9 | RAB14 | RAB14, member RAS oncogene family | 119396 | 119397 | 148180 | 26, 39, 11 |
| 9 | RABEPK | Rab9 effector protein with kelch motifs | 136933 | 119414 | 044574 | 9, 3, 13 |
| 9 | RAD23B | RAD23 homolog B, nucleotide excision repair protein | 119318 | 148143 | 136826 | 20, 23, 4 |
| 9 | RALGPS1 | Ral GEF with PH domain and SH3 binding motif 1 | 136828 | 177125 | 136859 | 35, 31, 37 |
| 9 | RALGDS | ral guanine nucleotide dissociation stimulator | 160271 | 170835 | 148288 | 41, 26, 31 |
| 9 | RANBP6 | RAN binding protein 6 | 137040 | 120215 | 137033 | 1, 26, 29 |
| 9 | RAPGEF1 | Rap guanine nucleotide exchange factor 1 | 107263 | 283526 | 160563 | 22, 21, 36 |
| 9 | RORB | RAR related orphan receptor B | 198963 | 135046 | 119121 | 14, 2, 32 |
| 9 | RASEF | RAS and EF-hand domain containing | 165105 | 214929 | 172159 | 4, 3, 40 |
| 9 | RRAGA | Ras related GTP binding A | 155876 | 155875 | 147874 | 1, 43, 32 |
| 9 | ROR2 | receptor tyrosine kinase like orphan receptor 2 | 169071 | 165030 | 090054 | |
| 9 | RMI1 | RecQ mediated genome instability 1 | 178966 | 165119 | 197506 | 22, 19, 13 |
| 9 | RGS3 | regulator of G-protein signaling 3 | 138835 | 157653 | 157657 | 34, 36, 6 |
| 9 | RFX3 | regulatory factor X3 | 080298 | 080608 | 279745 | 38, 26, 39 |
| 9 | RLN1 | relaxin 1 | 107018 | 107014 | 107020 | 33, 22, 5 |
| 9 | RLN2 | relaxin 2 | 107014 | 120211 | 107018 | 29, 31, 39 |
| 9 | RXRA | retinoid X receptor alpha | 186350 | 196363 | 130635 | 33, 1, 18 |
| 9 | RECK | reversion inducing cysteine rich protein with kazal motifs | 122707 | 243641 | 122694 | 1, 26, 32 |
| 9 | REXO4 | REX4 homolog, 3′-5′ exonuclease | 148300 | 198870 | 160323 | 9, 3, 45 |
| 9 | RGP1 | RGP1 homolog, RAB6A GEF complex partner 1 | 107185 | 070610 | 215183 | 27, 26, 17 |
| 9 | ARHGEF39 | Rho guanine nucleotide exchange factor 39 | 137135 | 159884 | 107159 | 1, 31, 35 |
| 9 | RFK | riboflavin kinase | 135002 | 099139 | 187210 | 26, 22, 8 |
| 9 | RPP25L | ribonuclease P/MRP subunit p25 like | 164967 | 122756 | 137100 | 29, 21, 15 |
| 9 | RPL12 | ribosomal protein L12 | 197958 | 196152 | 148356 | 41, 33, 3 |
| 9 | RPL35 | ribosomal protein L35 | 136942 | 136918 | 136950 | 33, 45, 43 |
| 9 | RPL7A | ribosomal protein L7a | 148303 | 148297 | 148290 | 41, 21, 43 |
| 9 | RPS6 | ribosomal protein S6 | 137154 | 137145 | 177076 | 41, 21, 43 |
| 9 | RIC1 | RIC1 homolog, RAB6A GEF complex partner 1 | 107036 | 197646 | 099219 | 11, 22, 26 |
| 9 | RC3H2 | ring finger and CCCH-type domains 2 | 056586 | 165204 | 186130 | 22, 1, 29 |
| 9 | RNF183 | ring finger protein 183 | 165188 | 136875 | 148225 | 11, 3, 17 |
| 9 | RNF20 | ring finger protein 20 | 155827 | 165152 | 198785 | 4, 30, 24 |
| 9 | RNF208 | ring finger protein 208 | 212864 | 283590 | 197191 | 14, 37, 6 |
| 9 | RNF224 | ring finger protein 224 | 233198 | 197191 | 198569 | 17, 32, |
| 9 | RNF38 | ring finger protein 38 | 137075 | 159921 | 165304 | 40, 38, 3 |
| 9 | RBM18 | RNA binding motif protein 18 | 119446 | 106852 | 148187 | 42, 29, 36 |
| 9 | POLR1E | RNA polymerase I subunit E | 137054 | 168795 | 147912 | |
| 9 | RCL1 | RNA terminal phosphate cyclase like 1 | 120158 | 147853 | 096968 | |
| 9 | RUSC2 | RUN and SH3 domain containing 2 | 198853 | 198722 | 215187 | 34, 26, 1 |
| 9 | SARDH | sarcosine dehydrogenase | 123453 | 123454 | 160293 | 19, 25, 17 |
| 9 | SEC16A | SEC16 homolog A, endoplasmic reticulum export factor | 148396 | 148384 | 148400 | 22, 37, 18 |
| 9 | SEC61B | Sec61 translocon beta subunit | 106803 | 119523 | 119508 | 43, 37, 31 |
| 9 | SECISBP2 | SECIS binding protein 2 | 187742 | 123975 | 187764 | 40, 3, 14 |
| 9 | SEMA4D | semaphorin 4D | 187764 | 187742 | 130222 | 22, 5, 29 |
| 9 | SETX | senataxin | 107290 | 196358 | 125482 | 22, 40, 1 |
| 9 | SPTLC1 | serine palmitoyltransferase long chain base subunit 1 | 090054 | 169071 | 196305 | 36, 16, 45 |
| 9 | SPINK4 | serine peptidase inhibitor, Kazal type 4 | 122711 | 086062 | 107262 | 16, 37, 10 |
| 9 | STKLD1 | serine/threonine kinase like domain containing 1 | 198870 | 148248 | 148300 | |
| 9 | SDCCAG3 | serologically defined colon cancer antigen 3 | 165689 | 165684 | 165688 | 9, 26, 35 |
| 9 | SET | SET nuclear proto-oncogene | 119335 | 119333 | 160447 | 11, 18, 6 |
| 9 | SH2D3C | SH2 domain containing 3C | 095370 | 160404 | 279363 | 39, 36, 22 |
| 9 | SHB | SH2 domain containing adaptor protein B | 107338 | 122696 | 137124 | 27, 17, 31 |
| 9 | SH3GL2 | SH3 domain containing GRB2 like 2, endophilin A1 | 107295 | 044459 | 178031 | 8, 31, 17 |
| 9 | SH3GLB2 | SH3 domain containing GRB2 like endophilin B2 | 148341 | 095319 | 148343 | 33, 3, 32 |
| 9 | SHC3 | SHC adaptor protein 3 | 148082 | 213694 | 123975 | 8, 2, 20 |
| 9 | SIGMAR1 | sigma non-opioid intracellular receptor 1 | 147955 | 205143 | 213930 | 35, 21, 9 |
| 9 | SIT1 | signaling threshold regulating transmembrane adaptor 1 | 137078 | 137101 | 159884 | 21, 41, 22 |
| 9 | SNAPC3 | small nuclear RNA activating complex polypeptide 3 | 164975 | 155158 | 164985 | 38, 40, 13 |
| 9 | SNAPC4 | small nuclear RNA activating complex polypeptide 4 | 165684 | 187796 | 165689 | 26, 21, 35 |
| 9 | SLC1A1 | solute carrier family 1 member 1 | 106688 | 107249 | 106686 | 22, 17, 9 |
| 9 | SLC2A6 | solute carrier family 2 member 6 | 160326 | 160325 | 187616 | 36, 45, 41 |
| 9 | SLC2A8 | solute carrier family 2 member 8 | 136856 | 136895 | 196152 | 38, 27, 6 |
| 9 | SLC24A2 | solute carrier family 24 member 2 | 155886 | 177076 | 171843 | 18, 2, 8 |
| 9 | SLC25A25 | solute carrier family 25 member 25 | 148339 | 171169 | 148334 | 26, 9, 27 |
| 9 | SLC25A51 | solute carrier family 25 member 51 | 122696 | 122741 | 107338 | 2, 42, 4 |
| 9 | SLC27A4 | solute carrier family 27 member 4 | 167114 | 167113 | 167118 | 9, 24, 3 |
| 9 | SLC28A3 | solute carrier family 28 member 3 | 197506 | 178966 | 148053 | 42, 4, 24 |
| 9 | SLC31A1 | solute carrier family 31 member 1 | 136868 | 119321 | 176386 | 3, 30, 42 |
| 9 | SLC31A2 | solute carrier family 31 member 2 | 136867 | 136866 | 119321 | 34, 29, 32 |
| 9 | SLC34A3 | solute carrier family 34 member 3 | 198569 | 233198 | 188229 | 17, |
| 9 | SLC35D2 | solute carrier family 35 member D2 | 130958 | 130948 | 165244 | |
| 9 | SLC44A1 | solute carrier family 44 member 1 | 070214 | 165029 | 106701 | 11, 26, 10 |
| 9 | SLC46A2 | solute carrier family 46 member 2 | 119457 | 148158 | 228623 | 39, 20, 5 |
| 9 | SNX30 | sorting nexin family member 30 | 148158 | 148153 | 119457 | 22, 26, 35 |
| 9 | SPATA31A1 | SPATA31 subfamily A member 1 | 204849 | 106714 | 183148 | |
| 9 | SPATA31A3 | SPATA31 subfamily A member 3 | 275969 | 274349 | 260691 | 38, |
| 9 | SPATA31A5 | SPATA31 subfamily A member 5 | 276581 | 277737 | 276040 | |
| 9 | SPATA31A6 | SPATA31 subfamily A member 6 | 185775 | 154529 | 277737 | 38, |
| 9 | SPATA31A7 | SPATA31 subfamily A member 7 | 276040 | 276581 | 283378 | |
| 9 | SPATA31D1 | SPATA31 subfamily D member 1 | 214929 | 186788 | 165105 | 38, |
| 9 | SPATA31D3 | SPATA31 subfamily D member 3 | 186788 | 189357 | 214929 | 25, 38, |
| 9 | SPATA31D4 | SPATA31 subfamily D member 4 | 189357 | 196781 | 186788 | 38, |
| 9 | SPATA31E1 | SPATA31 subfamily E member 1 | 177992 | 135047 | 283205 | 38, |
| 9 | SPTAN1 | spectrin alpha, non-erythrocytic 1 | 197694 | 119392 | 119333 | 26, 1, 13 |
| 9 | SPACA9 | sperm acrosome associated 9 | 165698 | 165695 | 165699 | |
| 9 | SPAG8 | sperm associated antigen 8 | 137098 | 159899 | 137133 | 38, 27, 20 |
| 9 | STRBP | spermatid perinuclear RNA binding protein | 165209 | 188394 | 148204 | 22, 41, 16 |
| 9 | SOHLH1 | spermatogenesis and oogenesis specific basic helix-loop-helix 1 | 165643 | 148386 | 107147 | 38, 8, |
| 9 | SPATA6L | spermatogenesis associated 6 like | 106686 | 106688 | 205808 | 4, 38, 37 |
| 9 | STPG3 | sperm-tail PG-rich repeat containing 3 | 197768 | 188163 | 188986 | |
| 9 | S1PR3 | sphingosine-1-phosphate receptor 3 | 213694 | 186354 | 148082 | 42, 33, 39 |
| 9 | SPIN1 | spindlin 1 | 106723 | 156345 | 130045 | 31, 11, 8 |
| 9 | SYK | spleen associated tyrosine kinase | 165025 | 165023 | 148090 | 41, 22, 29 |
| 9 | SPOUT1 | SPOUT domain containing methyltransferase 1 | 198917 | 167136 | 171097 | 37, 9, 41 |
| 9 | SSNA1 | SS nuclear autoantigen 1 | 176101 | 176248 | 176058 | 9, 41, 21 |
| 9 | ST6GALNAC4 | ST6 N-acetylgalactosaminide alpha-2,6-sialyltransferase 4 | 136840 | 160408 | 167103 | 41, 9, 28 |
| 9 | ST6GALNAC6 | ST6 N-acetylgalactosaminide alpha-2,6-sialyltransferase 6 | 160408 | 106992 | 136840 | |
| 9 | SAXO1 | stabilizer of axonemal microtubules 1 | 155875 | 178031 | 155876 | |
| 9 | STOM | stomatin | 148175 | 283430 | 136848 | 39, 32, 1 |
| 9 | STOML2 | stomatin like 2 | 165283 | 165282 | 005238 | 3, 28, 35 |
| 9 | SMC2 | structural maintenance of chromosomes 2 | 136824 | 155833 | 186881 | 34, 22, 4 |
| 9 | SMC5 | structural maintenance of chromosomes 5 | 198887 | 165072 | 119138 | 11, 25, 24 |
| 9 | SAPCD2 | suppressor APC domain containing 2 | 186193 | 054179 | 197355 | 34, 26, 9 |
| 9 | SCAI | suppressor of cancer cell invasion | 173611 | 136935 | 119414 | 39, 24, 31 |
| 9 | SURF1 | SURF1, cytochrome c oxidase assembly factor | 148290 | 148303 | 148291 | 1, 43, 6 |
| 9 | SURF2 | surfeit 2 | 148291 | 148290 | 148248 | 3, 11, 9 |
| 9 | SURF4 | surfeit 4 | 148248 | 148291 | 198870 | 3, 39, 2 |
| 9 | SURF6 | surfeit 6 | 148296 | 175164 | 148297 | 21, 41, 3 |
| 9 | SUSD1 | sushi domain containing 1 | 106868 | 148154 | 119314 | 45, 5, 11 |
| 9 | SUSD3 | sushi domain containing 3 | 157303 | 127084 | 165233 | 22, 18, 21 |
| 9 | SVEP1 | sushi, von Willebrand factor type A, EGF and pentraxin domain containing 1 | 165124 | 204193 | 030304 | 32, 1, 30 |
| 9 | SMARCA2 | SWI/SNF related, matrix associated, actin dependent regulator of chromatin, | 080503 | 173253 | 147852 | 29, 15, 23 |
| | | subfamily a, member 2 | | | | |
| 9 | SWI5 | SWI5 homologous recombination repair protein | 175854 | 167110 | 167112 | 7, 1, 3 |
| 9 | STX17 | syntaxin 17 | 136874 | 119508 | 023318 | 26, 22, 1 |
| 9 | STXBP1 | syntaxin binding protein 1 | 136854 | 136830 | 187024 | 8, 3, 26 |
| 9 | TRBV20OR9-2 | T cell receptor beta variable 20/OR9-2 (non-functional) | 205274 | 230453 | 183938 | |
| 9 | TRBV21OR9-2 | T cell receptor beta variable 21/OR9-2 (pseudogene) | 183938 | 205274 | 229063 | |
| 9 | TRBV23OR9-2 | T cell receptor beta variable 23/OR9-2 (non-functional) | 229063 | 183938 | 010438 | |
| 9 | TAL2 | TAL bHLH transcription factor 2 | 186051 | 106692 | 095209 | 22, |
| 9 | TLN1 | talin 1 | 137076 | 198467 | 107175 | 18, 40, 36 |
| 9 | TPRN | taperin | 176058 | 176101 | 187713 | 3, 16, 9 |
| 9 | TAF1L | TATA-box binding protein associated factor 1 like | 122728 | 241043 | 188133 | 40, 16, 25 |
| 9 | TBC1D13 | TBC1 domain family member 13 | 107021 | 160445 | 167136 | 9, 34, 15 |
| 9 | TBC1D2 | TBC1 domain family member 2 | 095383 | 106789 | 136928 | 24, 39, 4 |
| 9 | TEK | TEK receptor tyrosine kinase | 120156 | 184434 | 120160 | 42, 11, 45 |
| 9 | TNC | tenascin C | 041982 | 106952 | 173077 | |
| 9 | TEX10 | testis expressed 10 | 136891 | 119509 | 066697 | 24, 38, 39 |
| 9 | TESK1 | testis-specific kinase 1 | 107140 | 215187 | 137101 | 1, 43, 21 |
| 9 | TTC16 | tetratricopeptide repeat domain 16 | 167094 | 160401 | 160404 | 38, 27, 42 |
| 9 | TTC39B | tetratricopeptide repeat domain 39B | 155158 | 164946 | 164975 | 13, 11, 38 |
| 9 | TXN | thioredoxin | 136810 | 188959 | 204193 | 7, 19, 10 |
| 9 | TXNDC8 | thioredoxin domain containing 8 | 204193 | 136810 | 165124 | 38, |
| 9 | TSTD2 | thiosulfate sulfurtransferase like domain containing 2 | 136925 | 136842 | 136937 | 1, 26, 42 |
| 9 | TJP2 | tight junction protein 2 | 119139 | 165060 | 283143 | 40, 29, 16 |
| 9 | TRAF1 | TNF receptor associated factor 1 | 056558 | 119403 | 106804 | 43, 18, 20 |
| 9 | TRAF2 | TNF receptor associated factor 2 | 127191 | 107223 | 159069 | 36, 39, 9 |
| 9 | TLR4 | toll like receptor 4 | 136869 | 119401 | 078725 | 42, 7, 5 |
| 9 | TOPORS | TOP1 binding arginine/serine rich protein | 197579 | 107201 | 235453 | 45, 31, 39 |
| 9 | TOPORS-AS1 | TOPORS antisense RNA 1 | 235453 | 197579 | 165264 | |
| 9 | TOR1A | torsin family 1 member A | 136827 | 136816 | 136819 | 11, 9, 39 |
| 9 | TOR1B | torsin family 1 member B | 136816 | 148344 | 136827 | 35, 1, 33 |
| 9 | TOR2A | torsin family 2 member A | 160404 | 167094 | 095370 | 21, 1, 3 |
| 9 | TOR4A | torsin family 4 member A | 198113 | 188986 | 198435 | 22, 37, 42 |
| 9 | TTF1 | transcription termination factor 1 | 125482 | 107290 | 188523 | 13, 26, 34 |
| 9 | TLE1 | transducin like enhancer of split 1 | 196781 | 106829 | 189357 | |
| 9 | TLE4 | transducin like enhancer of split 4 | 106829 | 135069 | 196781 | 22, 38, 29 |
| 9 | TGFBR1 | transforming growth factor beta receptor 1 | 106799 | 204291 | 119523 | 31, 11, 2 |
| 9 | TRPM3 | transient receptor potential cation channel subfamily M member 3 | 083067 | 119138 | 135048 | 14, 31, 24 |
| 9 | TRPM6 | transient receptor potential cation channel subfamily M member 6 | 119121 | 198963 | 135045 | 17, 16, 42 |
| 9 | TOMM5 | translocase of outer mitochondrial membrane 5 | 175768 | 256966 | 255872 | 30, 34, 28 |
| 9 | TMC1 | transmembrane channel like 1 | 165091 | 107372 | 165092 | 7, 17, 33 |
| 9 | TMEM141 | transmembrane protein 141 | 244187 | 177984 | 272896 | 29, 33, 34 |
| 9 | TMEM2 | transmembrane protein 2 | 135048 | 083067 | 107362 | |
| 9 | TMEM203 | transmembrane protein 203 | 187713 | 176058 | 188566 | 22, 9, 37 |
| 9 | TMEM210 | transmembrane protein 210 | 185863 | 261793 | 176248 | |
| 9 | TMEM215 | transmembrane protein 215 | 188133 | 122728 | 137074 | 14, 38, 12 |
| 9 | TMEM245 | transmembrane protein 245 | 106771 | 119326 | 260230 | 29, 2, 26 |
| 9 | TMEM246 | transmembrane protein 246 | 165152 | 136872 | 155827 | 29, 28, 8 |
| 9 | TMEM252 | transmembrane protein 252 | 181778 | 154330 | 107242 | 17, 16, |
| 9 | TMEM261 | transmembrane protein 261 | 137038 | 107077 | 153707 | 29, 31, 3 |
| 9 | TMEM268 | transmembrane protein 268 | 157693 | 136888 | 230054 | |
| 9 | TMEM38B | transmembrane protein 38B | 095209 | 186051 | 148143 | 29, 38, 2 |
| 9 | TMEM8B | transmembrane protein 8B | 137103 | 137133 | 204930 | 40, 26, 38 |
| 9 | TMEM8C | transmembrane protein 8C | 187616 | 160326 | 197859 | |
| 9 | TMEFF1 | transmembrane protein with EGF like and two follistatin like domains 1 | 241697 | 251349 | 170681 | 45, 43, 26 |
| 9 | TRIM14 | tripartite motif containing 14 | 106785 | 095380 | 106789 | 39, 36, 24 |
| 9 | TRIM32 | tripartite motif containing 32 | 119401 | 148219 | 136869 | 43, 32, 35 |
| 9 | TRMT10B | tRNA methyltransferase 10B | 165275 | 070601 | 107371 | 1, 3, 27 |
| 9 | TRMO | tRNA methyltransferase O | 136932 | 178919 | 136929 | 35, 40, 13 |
| 9 | TMOD1 | tropomodulin 1 | 136842 | 196116 | 136925 | 15, 1, 11 |
| 9 | TPM2 | tropomyosin 2 (beta) | 198467 | 107159 | 137076 | 25, 43, 44 |
| 9 | TRUB2 | TruB pseudouridine synthase family member 2 | 167112 | 175854 | 167113 | 35, 31, 41 |
| 9 | TSC1 | tuberous sclerosis 1 | 165699 | 165698 | 165702 | 36, 23, 14 |
| 9 | TUBB4B | tubulin beta 4B class IVb | 188229 | 198569 | 188163 | 34, 21, 9 |
| 9 | TTLL11 | tubulin tyrosine liqase like 11 | 175764 | 136848 | 119421 | 38, 33, 9 |
| 9 | TDRD7 | tudor domain containinq 7 | 196116 | 197816 | 136842 | 14, 26, 1 |
| 9 | TNFSF15 | tumor necrosis factor superfamily member 15 | 181634 | 230601 | 106952 | 5, 32, 40 |
| 9 | TNFSF8 | tumor necrosis factor superfamily member 8 | 106952 | 181634 | 041982 | 39, 5, 22 |
| 9 | TPD52L3 | tumor protein D52 like 3 | 170777 | 137033 | 147854 | 38, 33, 8 |
| 9 | TUSC1 | tumor suppressor candidate 1 | 198680 | 205442 | 120159 | 31, 13, 22 |
| 9 | TYRP1 | tyrosinase related protein 1 | 107165 | 153707 | 153714 | 14, 11, 15 |
| 9 | UBAC1 | UBA domain containing 1 | 130560 | 130559 | 148411 | 21, 26, 32 |
| 9 | UBQLN1 | ubiquilin 1 | 135018 | 148057 | 165113 | 29, 22, 17 |
| 9 | UBAP1 | ubiquitin associated protein 1 | 165006 | 198876 | 186638 | 24, 41, 45 |
| 9 | UBAP2 | ubiquitin associated protein 2 | 137073 | 107341 | 198876 | 43, 21, 39 |
| 9 | UBE2R2 | ubiquitin conjugating enzyme E2 R2 | 107341 | 010438 | 137073 | 20, 15, 6 |
| 9 | UHRF2 | ubiquitin like with PHD and ring finger domains 2 | 147854 | 170777 | 178445 | 7, 22, 45 |
| 9 | URM1 | ubiquitin related modifier 1 | 167118 | 167114 | 167123 | 34, 41, 21 |
| 9 | USP20 | ubiquitin specific peptidase 20 | 136878 | 136819 | 187239 | 26, 7, 21 |
| 9 | UGCG | UDP-glucose ceramide glucosyltransferase | 148154 | 165181 | 106868 | |
| 9 | UAP1L1 | UDP-N-acetylglucosamine pyrophosphorylase 1 like 1 | 197355 | 186193 | 177239 | 34, 18, 21 |
| 9 | UNC13B | unc-13 homolog B | 198722 | 005238 | 198853 | 43, 35, 26 |
| 9 | UCK1 | uridine-cytidine kinase 1 | 130717 | 130714 | 283526 | 41, 9, 26 |
| 9 | VPS13A | vacuolar protein sorting 13 homolog A | 197969 | 204612 | 156049 | 1, 43, 33 |
| 9 | VCP | valosin containing protein | 165280 | 174038 | 221829 | 24, 21, 42 |
| 9 | VAV2 | vav guanine nucleotide exchange factor 2 | 160293 | 123453 | 235106 | 22, 13, 26 |
| 9 | VLDLR | very low density lipoprotein receptor | 147852 | 080503 | 168263 | 1, 15, 40 |
| 9 | WASH1 | WAS protein family homolog 1 | 181404 | 107175 | 170122 | |
| 9 | WDR31 | WD repeat domain 31 | 148225 | 165188 | 119411 | 29, 34, 38 |
| 9 | WDR34 | WD repeat domain 34 | 119333 | 197694 | 119335 | 34, 9, 20 |
| 9 | WDR38 | WD repeat domain 38 | 136918 | 185585 | 136942 | 43, 38, 27 |
| 9 | WDR5 | WD repeat domain 5 | 196363 | 169925 | 186350 | 21, 22, 39 |
| 9 | WHRN | whirlin | 095397 | 106948 | 136888 | |
| 9 | WNK2 | WNK lysine deficient protein kinase 2 | 165238 | 131669 | 204352 | 42, 21, 26 |
| 9 | XPA | XPA, DNA damage recognition and repair factor | 136936 | 136937 | 178919 | 3, 31, 41 |
| 9 | ZFP37 | ZFP37 zinc finger protein | 136866 | 228623 | 136867 | 30, 36, 6 |
| 9 | ZFAND5 | zinc finger AN1-type containing 5 | 107372 | 119125 | 165091 | 11, 31, 29 |
| 9 | ZBTB26 | zinc finger and BTB domain containing 26 | 171448 | 186130 | 011454 | 29, 38, 39 |
| 9 | ZBTB34 | zinc finger and BTB domain containing 34 | 177125 | 169155 | 136828 | 11, 22, 36 |
| 9 | ZBTB43 | zinc finger and BTB domain containing 43 | 169155 | 136944 | 177125 | 13, 20, 4 |
| 9 | ZBTB5 | zinc finger and BTB domain containing 5 | 168795 | 137106 | 137054 | 15, 6, 11 |
| 9 | ZBTB6 | zinc finger and BTB domain containing 6 | 186130 | 056586 | 171448 | 9, 26, 13 |
| 9 | ZCCHC6 | zinc finger CCHC-type containing 6 | 083223 | 135070 | 180447 | 31, 26, 23 |
| 9 | ZCCHC7 | zinc finger CCHC-type containing 7 | 147905 | 196092 | 137106 | 7, 41, 18 |
| 9 | ZDHHC12 | zinc finger DHHC-type containing 12 | 160446 | 160447 | 160445 | 3, 13, 9 |
| 9 | ZDHHC21 | zinc finger DHHC-type containing 21 | 175893 | 147862 | 147869 | 45, 25, 7 |
| 9 | ZMYND19 | zinc finger MYND-type containing 19 | 165724 | 148399 | 197070 | 9, 3, 38 |
| 9 | ZNF169 | zinc finger protein 169 | 175787 | 158079 | 130950 | 42, 30, 22 |
| 9 | ZNF189 | zinc finger protein 189 | 136870 | 136897 | 136872 | 13, 11, 31 |
| 9 | ZNF367 | zinc finger protein 367 | 165244 | 130958 | 130956 | 22, 3, 4 |
| 9 | ZNF462 | zinc finger protein 462 | 148143 | 095209 | 119318 | 33, 18, 2 |
| 9 | ZNF483 | zinc finger protein 483 | 173258 | 136813 | 283263 | 40, 38, 37 |
| 9 | ZNF484 | zinc finger protein 484 | 127081 | 185963 | 127084 | 1, 30, 42 |
| 9 | ZNF510 | zinc finger protein 510 | 081386 | 158122 | 196597 | 3, 36, 33 |
| 9 | ZNF618 | zinc finger protein 618 | 157657 | 138835 | 106927 | 43, 14, 27 |
| 9 | ZNF658 | zinc finger protein 658 | 274349 | 276203 | 275969 | |
| 9 | ZNF782 | zinc finger protein 782 | 196597 | 081386 | 196312 | 42, 15, 38 |
| 9 | ZNF79 | zinc finger protein 79 | 196152 | 136856 | 197958 | 13, 2, 20 |
| 9 | ZNF883 | zinc finger protein 883 | 228623 | 119457 | 136866 | 10, 25, 22 |
| 9 | ZER1 | zyg-11 related cell cycle regulator | 160445 | 160446 | 107021 | 27, 38, 29 |

Table 5 provides the gene information for the genes found on Chromosomes 10 and 11.

TABLE 5
Genes on Chromosomes 10 and 11

| | | | | Up | Down | Tissue |
|---|---|---|---|---|---|---|
| Chr. | Gene Symbol | Gene Name | ENSG | ENSG | ENSG | Code |
| 10 | ADO | 2-aminoethanethiol dioxygenase | 181915 | 138311 | 122877 | 22, 38, 11 |
| 10 | HACD1 | 3-hydroxyacyl-CoA dehydratase 1 | 165996 | 148488 | 136738 | 45, 11, 2 |
| 10 | PAPSS2 | 3′-phosphoadenosine 5′-phosphosulfate synthase 2 | 198682 | 107789 | 138138 | 45, 10, 6 |
| 10 | HOGA1 | 4-hydroxy-2-oxoglutarate aldolase 1 | 241935 | 165887 | 249967 | 2, 34, 17 |
| 10 | HTR7 | 5-hydroxytryptamine receptor 7 | 148680 | 138182 | 148688 | 6, 38, 8 |
| 10 | NT5C2 | 5′-nucleotidase, cytosolic II | 076685 | 148842 | 235376 | 42, 39, 24 |
| 10 | PFKFB3 | 6-phosphofructo-2-kinase/fructose-2,6-biphosphatase 3 | 170525 | 134453 | 065675 | 42, 8, 45 |
| 10 | ABI1 | abl interactor 1 | 136754 | 148459 | 107890 | 39, 4, 45 |
| 10 | ABLIM1 | actin binding LIM protein 1 | 099204 | 169129 | 151553 | 30, 26, 41 |
| 10 | AFAP1L2 | actin filament associated protein 1 like 2 | 169129 | 165816 | 099204 | 40, 29, 36 |
| 10 | ACTA2 | actin, alpha 2, smooth muscle, aorta | 107796 | 138134 | 026103 | 43, 1, 45 |
| 10 | ASCC1 | activating signal cointegrator 1 complex subunit 1 | 138303 | 107742 | 166295 | 13, 43, 36 |
| 10 | ACBD5 | acyl-CoA binding domain containing 5 | 107897 | 120539 | 182077 | 42, 43, 8 |
| 10 | ACBD7 | acyl-CoA binding domain containing 7 | 176244 | 152463 | 152464 | |
| 10 | ACADSB | acyl-CoA dehydrogenase, short/branched chain | 196177 | 095574 | 188620 | 17, 19, 4 |
| 10 | ACSL5 | acyl-CoA synthetase long-chain family member 5 | 197142 | 119913 | 023041 | 16, 42, 37 |
| 10 | ACSM6 | acyl-CoA synthetase medium-chain family member 6 | 173124 | 138115 | 107438 | 39, 15, 35 |
| 10 | ADAM12 | ADAM metallopeptidase domain 12 | 148848 | 203780 | 154493 | 32, 6, 18 |
| 10 | ADAM8 | ADAM metallopeptidase domain 8 | 151651 | 151650 | 130640 | 7, 39, 36 |
| 10 | ADAMTS14 | ADAM metallopeptidase with thrombospondin type 1 motif 14 | 138316 | 180644 | 166220 | 6, 42, 15 |
| 10 | AP3M1 | adaptor related protein complex 3 mu 1 subunit | 185009 | 035403 | 156110 | 24, 42, 13 |
| 10 | ADD3 | adducin 3 | 148700 | 108039 | 119950 | 11, 22, 1 |
| 10 | ADARB2 | adenosine deaminase, RNA specific B2 (inactive) | 185736 | 047056 | 067057 | 8, 31, 14 |
| 10 | ADK | adenosine kinase | 156110 | 185009 | 156650 | 10, 23, 25 |
| 10 | ADGRA1 | adhesion G protein-coupled receptor A1 | 197177 | 171811 | 171798 | 16, 8, 17 |
| 10 | ADIRF | adipogenesis regulatory factor | 148671 | 173267 | 188100 | 1, 27, 33 |
| 10 | ARL3 | ADP ribosylation factor like GTPase 3 | 138175 | 171206 | 156398 | 29, 15, 34 |
| 10 | ARL5B | ADP ribosylation factor like GTPase 5B | 165997 | 241058 | 204740 | 12, 42, 24 |
| 10 | ADRA2A | adrenoceptor alpha 2A | 150594 | 108061 | 119927 | 11, 26, 28 |
| 10 | ADRB1 | adrenoceptor beta 1 | 043591 | 196865 | 165813 | 22, 15, 32 |
| 10 | ARMS2 | age-related maculopathy susceptibility 2 | 254636 | 107679 | 166033 | 10, 32, 38 |
| 10 | ALDH18A1 | aldehyde dehydrogenase 18 family member A1 | 059573 | 095637 | 119977 | 42, 11, 24 |
| 10 | AKR1C1 | aldo-keto reductase family 1 member C1 | 187134 | 165568 | 151632 | 24, 19, 42 |
| 10 | AKR1C2 | aldo-keto reductase family 1 member C2 | 151632 | 187134 | 196139 | 13, 24, 4 |
| 10 | AKR1C3 | aldo-keto reductase family 1 member C3 | 196139 | 151632 | 198610 | 29, 4, 19 |
| 10 | AKR1C4 | aldo-keto reductase family 1 member C4 | 198610 | 196139 | 178473 | 19, 10, 3 |
| 10 | AKR1E2 | aldo-keto reductase family 1 member E2 | 165568 | 067082 | 187134 | 38, 45, 10 |
| 10 | APBB1IP | amyloid beta precursor protein binding family B member 1 interacting protein | 077420 | 136750 | 148459 | 39, 41, 7 |
| 10 | ANAPC16 | anaphase promoting complex subunit 16 | 166295 | 138303 | 168209 | 34, 11, 7 |
| 10 | ANK3 | ankyrin 3 | 151150 | 108091 | 170312 | |
| 10 | ASB13 | ankyrin repeat and SOCS box containing 13 | 196372 | 178363 | 108021 | 3, 34, 22 |
| 10 | ANKRD1 | ankyrin repeat domain 1 | 148677 | 148688 | 180628 | 15, 45, 18 |
| 10 | ANKRD16 | ankyrin repeat domain 16 | 134461 | 057608 | 134452 | 31, 5, 38 |
| 10 | ANKRD2 | ankyrin repeat domain 2 | 165887 | 165886 | 241935 | 25, 15, 7 |
| 10 | ANKRD22 | ankyrin repeat domain 22 | 152766 | 173239 | 138134 | 30, 4, 37 |
| 10 | ANKRD26 | ankyrin repeat domain 26 | 107890 | 136754 | 136758 | 11, 21, 13 |
| 10 | ANKRD30A | ankyrin repeat domain 30A | 148513 | 177283 | 256892 | 23, 5, |
| 10 | ANXA11 | annexin A11 | 122359 | 189129 | 151224 | 1, 21, 27 |
| 10 | ANXA7 | annexin A7 | 138279 | 156042 | 166343 | 30, 29, 7 |
| 10 | ANXA8 | annexin A8 | 265190 | 265763 | 189090 | |
| 10 | ANXA8L1 | annexin A8-like 1 | 264230 | 279058 | 204174 | 35, 15, 44 |
| 10 | ANTXRL | anthrax toxin receptor-like | 274209 | 263639 | 279058 | 38, 25, |
| 10 | A1CF | APOBEC1 complementation factor | 148584 | 204147 | 185532 | 19, 37, 28 |
| 10 | AIFM2 | apoptosis inducing factor, mitochondria associated 2 | 042286 | 099284 | 156521 | 1, 9, 20 |
| 10 | ALOX5 | arachidonate 5-lipoxygenase | 012779 | 256574 | 165406 | 22, 41, 7 |
| 10 | AGAP14 | ArfGAP with GTPase domain, ankyrin repeat and PH domain 14 | 279058 | 274209 | 264230 | |
| 10 | AGAP4 | ArfGAP with GTPase domain, ankyrin repeat and PH domain 4 | 188234 | 172661 | 265354 | |
| 10 | AGAP5 | ArfGAP with GTPase domain, ankyrin repeat and PH domain 5 | 172650 | 166317 | 176986 | 18, 4, 7 |
| 10 | AGAP6 | ArfGAP with GTPase domain, ankyrin repeat and PH domain 6 | 204149 | 204152 | 099290 | 18, 33, 40 |
| 10 | AGAP9 | ArfGAP with GTPase domain, ankyrin repeat and PH domain 9 | 204172 | 189090 | 277758 | |
| 10 | AVPI1 | arginine vasopressin induced 1 | 119986 | 155252 | 155254 | 9, 31, 1 |
| 10 | ATE1 | arginyltransferase 1 | 107669 | 066468 | 107672 | 39, 42, 38 |
| 10 | ARHGAP19- | ARHGAP19-SLIT1 readthrough (NMD candidate) | 269891 | 187122 | 213390 | |
| | SLIT1 | | | | | |
| 10 | ARMC3 | armadillo repeat containing 3 | 165309 | 150867 | 148450 | 38, 31, 42 |
| 10 | ARMC4 | armadillo repeat containing 4 | 169126 | 150051 | 150054 | 10, 42, 38 |
| 10 | ACTR1A | ARP1 actin-related protein 1 homolog A, centractin alpha | 138107 | 138111 | 107882 | 8, 39, 38 |
| 10 | AS3MT | arsenite methyltransferase | 214435 | 270316 | 148842 | 2, 17, 13 |
| 10 | ATOH7 | atonal bHLH transcription factor 7 | 179774 | 138347 | 108187 | 14, |
| 10 | ABCC2 | ATP binding cassette subfamily C member 2 | 023839 | 014919 | 107554 | 19, 17, 37 |
| 10 | ATP5C1 | ATP synthase, H+ transporting, mitochondrial F1 complex, gamma polypeptide 1 | 165629 | 151657 | 165632 | 4, 34, 7 |
| 10 | ATAD1 | ATPase family, AAA domain containing 1 | 138138 | 198682 | 227268 | 29, 42, 30 |
| 10 | ARID5B | AT-rich interaction domain 5B | 150347 | 183346 | 182010 | 11, 18, 13 |
| 10 | ATRNL1 | attractin like 1 | 107518 | 165832 | 151892 | 26, 1, 30 |
| 10 | BBIP1 | BBSome interacting protein 1 | 214413 | 150593 | 108061 | |
| 10 | BLNK | B-cell linker | 095585 | 177853 | 107447 | 30, 41, 22 |
| 10 | BAG3 | BCL2 associated athanogene 3 | 151929 | 151923 | 198825 | 1, 45, 29 |
| 10 | BNIP3 | BCL2 interacting protein 3 | 176171 | 175470 | 188385 | 6, 22, 1 |
| 10 | BEND7 | BEN domain containing 7 | 165626 | 086475 | 165630 | 29, 1, 17 |
| 10 | BTRC | beta-transducin repeat containing E3 ubiquitin protein ligase | 166167 | 138136 | 166171 | 29, 2, 25 |
| 10 | BICC1 | BicC family RNA binding protein 1 | 122870 | 108064 | 165443 | 17, 19, 14 |
| 10 | BLOC1S2 | biogenesis of lysosomal organelles complex 1 subunit 2 | 196072 | 095485 | 107593 | 11, 31, 22 |
| 10 | BORCS7 | BLOC-1 related complex subunit 7 | 166275 | 148795 | 270316 | 29, 2, 43 |
| 10 | BMI1 | BMI1 proto-oncogene, polycomb ring finger | 168283 | 269897 | 077327 | 29, 31, 13 |
| 10 | BAMBI | BMP and activin membrane bound inhibitor | 095739 | 095787 | 120563 | 11, 12, 35 |
| 10 | BMS1 | BMS1, ribosome biogenesis factor | 165733 | 196693 | 165731 | 25, 30, 1 |
| 10 | BMPR1A | bone morphogenetic protein receptor type 1A | 107779 | 122367 | 173269 | 42, 3, 29 |
| 10 | BORCS7-ASMT | BORCS7-ASMT readthrough (NMD candidate) | 270316 | 166275 | 214435 | 2, 17, 13 |
| 10 | BCCIP | BRCA2 and CDKN1A interacting protein | 107949 | 188690 | 089876 | 17, 6, 30 |
| 10 | BTBD16 | BTB domain containing 16 | 138152 | 138162 | 107679 | 15, 4, 38 |
| 10 | BTAF1 | B-TFIID TATA-box binding protein associated factor 1 | 095564 | 174721 | 107864 | 39, 42, 4 |
| 10 | BUB3 | BUB3, mitotic checkpoint protein | 154473 | 188816 | 154478 | 30, 22, 24 |
| 10 | CDH23 | cadherin related 23 | 107736 | 198246 | 214688 | 14, 36, 3 |
| 10 | CDHR1 | cadherin related family member 1 | 148600 | 188373 | 204033 | 14, 35, 16 |
| 10 | CALHM1 | calcium homeostasis modulator 1 | 185933 | 138172 | 183128 | 8, |
| 10 | CALHM2 | calcium homeostasis modulator 2 | 138172 | 148843 | 185933 | 13, 30, 15 |
| 10 | CALHM3 | calcium homeostasis modulator 3 | 183128 | 185933 | 107954 | 21, 9, 32 |
| 10 | CACNB2 | calcium voltage-gated channel auxiliary subunit beta 2 | 165995 | 148482 | 241058 | 31, 15, 19 |
| 10 | CAMK1D | calcium/calmodulin dependent protein kinase ID | 183049 | 151465 | 151468 | 1, 26, 20 |
| 10 | CAMK2G | calcium/calmodulin dependent protein kinase II gamma | 148660 | 166507 | 122861 | 8, 16, 44 |
| 10 | CALY | calcyon neuron specific vesicular protein | 130643 | 283496 | 165828 | 31, 28, 14 |
| 10 | CALML3 | calmodulin like 3 | 178363 | 178372 | 196372 | 18, 35, 13 |
| 10 | CALML5 | calmodulin like 5 | 178372 | 173848 | 178363 | 23, 13, 35 |
| 10 | CREM | cAMP responsive element modulator | 095794 | 108094 | 108100 | 38, 5, 1 |
| 10 | CASC10 | cancer susceptibility candidate 10 | 204682 | 204683 | 180592 | |
| 10 | CHST15 | carbohydrate sulfotransferase 15 | 182022 | 121898 | 065154 | 22, 23, 36 |
| 10 | CHST3 | carbohydrate sulfotransferase 3 | 122863 | 197746 | 107742 | 43, 19, 34 |
| 10 | CPN1 | carboxypeptidase N subunit 1 | 120054 | 107554 | 107566 | 19, 35, 5 |
| 10 | CPXM2 | carboxypeptidase X, M14 family member 2 | 121898 | 154478 | 182022 | 1, 11, 31 |
| 10 | CRTAC1 | cartilage acidic protein 1 | 095713 | 155265 | 166024 | 10, 14, 17 |
| 10 | CASP7 | caspase 7 | 165806 | 197893 | 148735 | 45, 4, 42 |
| 10 | COMTD1 | catechol-O-methyltransferase domain containing 1 | 165644 | 165637 | 165655 | 41, 37, 28 |
| 10 | CTNNA3 | catenin alpha 3 | 183230 | 165476 | 198739 | 10, 8, 26 |
| 10 | CISD1 | CDGSH iron sulfur domain 1 | 122873 | 151151 | 072401 | 34, 7, 4 |
| 10 | CACUL1 | CDK2 associated cullin domain 1 | 151893 | 119973 | 188613 | 11, 27, 38 |
| 10 | CDC123 | cell division cycle 123 | 151465 | 165609 | 183049 | 13, 7, 3 |
| 10 | CCAR1 | cell division cycle and apoptosis regulator 1 | 060339 | 138336 | 165730 | 24, 42, 13 |
| 10 | CEP55 | centrosomal protein 55 | 138180 | 138119 | 186188 | 6, 22, 3 |
| 10 | CDNF | cerebral dopamine neurotrophic factor | 185267 | 065809 | 187522 | 42, 4, 38 |
| 10 | CH25H | cholesterol 25-hydroxylase | 138135 | 026103 | 107798 | 1, 2, 30 |
| 10 | CHAT | choline O-acetyltransferase | 070748 | 243251 | 187714 | 14, 8, 38 |
| 10 | CSGALNACT2 | chondroitin sulfate N-acetylgalactosaminyltransferase 2 | 169826 | 165731 | 198915 | 45, 5, 22 |
| 10 | C10orf10 | chromosome 10 open reading frame 10 | 165507 | 107551 | 165512 | 1, 4, 39 |
| 10 | C10orf105 | chromosome 10 open reading frame 105 | 214688 | 107736 | 107738 | 14, 10, 20 |
| 10 | C10orf107 | chromosome 10 open reading frame 107 | 183346 | 196932 | 150347 | 26, 10, 38 |
| 10 | C10orf11 | chromosome 10 open reading frame 11 | 148655 | 165655 | 281042 | |
| 10 | C10orf113 | chromosome 10 open reading frame 113 | 204683 | 078114 | 204682 | 23, 38, |
| 10 | C10orf120 | chromosome 10 open reading frame 120 | 183559 | 187908 | 138161 | |
| 10 | C10orf128 | chromosome 10 open reading frame 128 | 204161 | 172538 | 177354 | 1, 30, 26 |
| 10 | C10orf142 | chromosome 10 open reading frame 142 | 277288 | 169740 | 107562 | 38, 8, |
| 10 | C10orf35 | chromosome 10 open reading frame 35 | 171224 | 122859 | 197467 | 26, 31, 14 |
| 10 | C10orf53 | chromosome 10 open reading frame 53 | 178645 | 187714 | 197444 | 38, 20, 28 |
| 10 | C10orf54 | Chromosome 10 open reading frame 54 | 107738 | 214688 | 197746 | 19, 26, 32 |
| 10 | C10orf55 | Chromosome 10 open reading frame 55 | 222047 | 122861 | 035403 | 38, 15, 17 |
| 10 | C10orf62 | chromosome 10 open reading frame 62 | 203942 | 249967 | 171160 | 38, 8, |
| 10 | C10orf67 | chromosome 10 open reading frame 67 | 179133 | 168267 | 165312 | 17, 20, |
| 10 | C10orf71 | chromosome 10 open reading frame 71 | 177354 | 204161 | 165606 | 25, 15, |
| 10 | C10orf76 | chromosome 10 open reading frame 76 | 120029 | 120049 | 166189 | 39, 23, 18 |
| 10 | C10orf82 | chromosome 10 open reading frame 82 | 165863 | 266200 | 165868 | 38, 12, 33 |
| 10 | C10orf88 | chromosome 10 open reading frame 88 | 119965 | 203795 | 179988 | 26, 38, 25 |
| 10 | C10orf90 | chromosome 10 open reading frame 90 | 154493 | 148848 | 150760 | 18, 6, 35 |
| 10 | C10orf95 | Chromosome 10 open reading frame 95 | 120055 | 107874 | 138111 | 28, 20, 27 |
| 10 | C10orf99 | chromosome 10 open reading frame 99 | 188373 | 165678 | 148600 | 16, 27, 9 |
| 10 | CFAP43 | cilia and flagella associated protein 43 | 197748 | 156384 | 148834 | 42, 38, 26 |
| 10 | CFAP46 | cilia and flagella associated protein 46 | 171811 | 148826 | 197177 | |
| 10 | CFAP58 | cilia and flagella associated protein 58 | 120051 | 148841 | 156395 | 30, 38, 44 |
| 10 | CFAP70 | cilia and flagella associated protein 70 | 156042 | 182180 | 138279 | 31, 26, 38 |
| 10 | CLRN3 | clarin 3 | 180745 | 186766 | 132334 | 3, 19, 16 |
| 10 | CSTF2T | cleavage stimulation factor subunit 2 tau variant | 177613 | 185532 | 107984 | 29, 36, 39 |
| 10 | CC2D2B | coiled-coil and C2 domain containing 2B | 188649 | 270099 | 107443 | 14, 38, |
| 10 | CCDC172 | coiled-coil domain containing 172 | 182645 | 151892 | 203837 | 10, 38, 12 |
| 10 | CCDC186 | coiled-coil domain containing 186 | 165813 | 043591 | 095627 | |
| 10 | CCDC3 | coiled-coil domain containing 3 | 151468 | 183049 | 123240 | 1, 26, 31 |
| 10 | CCDC6 | coiled-coil domain containing 6 | 108091 | 227877 | 151150 | 4, 3, 31 |
| 10 | CCDC7 | coiled-coil domain containing 7 | 216937 | 120616 | 150076 | 4, 34, 38 |
| 10 | CCSER2 | coiled-coil serine rich protein 2 | 107771 | 148604 | 182771 | 15, 10, 26 |
| 10 | CHCHD1 | coiled-coil-helix-coiled-coil-helix domain containing 1 | 172586 | 196968 | 214655 | 3, 4, 31 |
| 10 | COL13A1 | collagen type XIII alpha 1 chain | 197467 | 171224 | 099284 | 27, 6, 12 |
| 10 | COL17A1 | collagen type XVII alpha 1 chain | 065618 | 065613 | 156384 | |
| 10 | COMMD3 | COMM dcmain ccntaining 3 | 148444 | 223601 | 269897 | 29, 7, 5 |
| 10 | COMMD3-BMI1 | COMMD3-BMI1 readthrough | 269897 | 148444 | 168283 | |
| 10 | C1QL3 | complement C1q like 3 | 165985 | 165983 | 148484 | 26, 8, |
| 10 | CHUK | conserved helix-loop-helix ubiquitous kinase | 213341 | 107566 | 095485 | 26, 7, 34 |
| 10 | COX15 | COX15, cytochrome c oxidase assembly homolog | 014919 | 119929 | 023839 | 29, 7, 42 |
| 10 | CTBP2 | C-terminal binding protein 2 | 175029 | 019995 | 175018 | 38, |
| 10 | CUZD1 | CUB and zena pellucida like domains 1 | 138161 | 183559 | 213185 | 4, 28, 19 |
| 10 | CUBN | cubilin | 107611 | 148484 | 107614 | 17, 38, 42 |
| 10 | CUEDC2 | CUE domain containing 2 | 107874 | 107872 | 120055 | 41, 3, 26 |
| 10 | CELF2 | CUGBP, Elav-like family member 2 | 048740 | 107485 | 148429 | 22, 1, 5 |
| 10 | CUL2 | cullin 2 | 108094 | 148498 | 095794 | 6, 24, 26 |
| 10 | CUTC | cutC copper transporter | 119929 | 198018 | 014919 | 25, 30, 2 |
| 10 | CWF19L1 | CWF19-like 1, cell cycle central (S. pombe) | 095485 | 213341 | 196072 | 45, 39, 5 |
| 10 | CXCL12 | C-X-C motif chemokine ligand 12 | 107562 | 277288 | 187783 | 26, 6, 1 |
| 10 | CNNM1 | cyclin and CBS domain divalent metal cation transport mediator 1 | 119946 | 172987 | 120053 | 38, 14, 8 |
| 10 | CNNM2 | cyclin and CBS domain divalent metal cation transport mediator 2 | 148842 | 214435 | 076685 | 41, 6, 16 |
| 10 | CDK1 | cyclin dependent kinase 1 | 170312 | 151150 | 072422 | 22, 13, 23 |
| 10 | CCNJ | cyclin J | 107443 | 188649 | 177853 | 3, 12, 45 |
| 10 | CCNY | cyclin Y | 108100 | 095794 | 177291 | 36, 38, 29 |
| 10 | CYP17A1 | cytochrome P450 family 17 subfamily A member 1 | 148795 | 166272 | 166275 | 2, 17, 40 |
| 10 | CYP2C18 | cytochrome P450 family 2 subfamily C member 18 | 108242 | 119969 | 276490 | 19, 24, 37 |
| 10 | CYP2C19 | cytochrome P450 family 2 subfamily C member 19 | 165841 | 276490 | 138109 | 19, |
| 10 | CYP2C8 | cytochrome P450 family 2 subfamily C member 8 | 138115 | 138109 | 173124 | 19, 23, 24 |
| 10 | CYP2C9 | cytochrome P450 family 2 subfamily C member 9 | 138109 | 165841 | 138115 | 19, 10, 23 |
| 10 | CYP2E1 | cytochrome P450 family 2 subfamily E member 1 | 130649 | 203772 | 171772 | 19, 5, 27 |
| 10 | CYP26A1 | cytochrome P450 family 26 subfamily A member 1 | 095596 | 187553 | 138119 | 12, 18, 3 |
| 10 | CYP26C1 | cytochrome P450 family 26 subfamily C member 1 | 187553 | 138190 | 095596 | 27, 8, |
| 10 | CPEB3 | cytoplasmic polyadenylation element binding protein 3 | 107864 | 095564 | 198060 | 28, 19, 38 |
| 10 | DHX32 | DEAH-box helicase 32 (putative) | 089876 | 107949 | 203780 | 40, 11, 23 |
| 10 | PDSS1 | decaprenyl diphosphate synthase subunit 1 | 148459 | 077420 | 136754 | 10, 16, 3 |
| 10 | DOCK1 | dedicator of cytokinesis 1 | 150760 | 154493 | 188916 | 29, 24, 45 |
| 10 | DHTKD1 | dehydrogenase E1 and transketolase domain containing 1 | 181192 | 151461 | 065665 | 24, 23, 9 |
| 10 | DMBT1 | deleted in malignant brain tumors 1 | 187908 | 166033 | 183559 | 42, 37, 16 |
| 10 | DPCD | deleted in primary ciliary dyskinesia homolog (mouse) | 166171 | 166167 | 166169 | 29, 3, 30 |
| 10 | DDX21 | DExD-box helicase 21 | 165732 | 107625 | 198954 | 21, 41, 19 |
| 10 | DDX50 | DExD-box helicase 50 | 107625 | 165730 | 165732 | 24, 30, 31 |
| 10 | DKK1 | dickkopf WNT signaling pathway inhibitor 1 | 107984 | 177613 | 165471 | 37, 40, 32 |
| 10 | DPYSL4 | dihydropyrimidinase like 4 | 151640 | 188385 | 165752 | 34, 14, 2 |
| 10 | DIP2C | disco interacting protein 2 homolog C | 151240 | 015171 | 180525 | 11, 35, 17 |
| 10 | DLG5 | discs large MAGUK scaffold protein 5 | 151208 | 156113 | 148606 | 34, 23, 32 |
| 10 | DCLRE1A | DNA cross-link repair 1A | 198924 | 148735 | 196865 | 42, 29, 35 |
| 10 | DCLRE1C | DNA cross-link repair 1C | 152457 | 152455 | 197889 | 22, 41, 18 |
| 10 | DDIT4 | DNA damage inducible transcript 4 | 168209 | 166295 | 148719 | 3, 9, 4 |
| 10 | DNTT | DNA nucleotidylexotransferase | 107447 | 095585 | 197430 | 39, 5, 22 |
| 10 | POLL | DNA polymerase lambda | 166169 | 166171 | 107829 | 43, 1, 36 |
| 10 | DNA2 | DNA replication helicase/nuclease 2 | 138346 | 204130 | 122912 | 22, 18, 7 |
| 10 | DNAJB12 | DnaJ heat shock protein family (Hsp40) member B12 | 148719 | 168209 | 107745 | 29, 34, 41 |
| 10 | DNAJC1 | DnaJ heat shock protein family (Hsp40) member C1 | 136770 | 078403 | 223601 | 43, 29, 18 |
| 10 | DNAJC12 | DnaJ heat shock protein family (Hsp40) member C12 | 108176 | 198739 | 096717 | 29, 34, 10 |
| 10 | DNAJC9 | DnaJ heat shock protein family (Hsp40) member C9 | 213551 | 138286 | 182180 | 11, 16, 4 |
| 10 | DRGX | dorsal root ganglia homeobox | 165606 | 177354 | 225830 | |
| 10 | DYDC1 | DPY30 domain containing 1 | 170788 | 151224 | 133665 | 38, 8, |
| 10 | DYDC2 | DPY30 domain containing 2 | 133665 | 170788 | 122378 | 20, 30, 35 |
| 10 | DUSP13 | dual specificity phosphatase 13 | 079393 | 188716 | 156671 | 25, 15, 7 |
| 10 | DUSP5 | dual specificity phosphatase 5 | 138166 | 119953 | 108055 | 6, 11, 35 |
| 10 | DUPD1 | dual specificity phosphatase and pro isomerase domain containing 1 | 188716 | 156650 | 079393 | |
| 10 | DNMBP | dynamin binding protein | 107554 | 023839 | 120054 | 29, 3, 34 |
| 10 | EBF3 | early B-cell factor 3 | 108001 | 170430 | 237489 | 29, 14, 15 |
| 10 | EGR2 | early growth response 2 | 122877 | 181915 | 148572 | 40, 24, 5 |
| 10 | ECD | ecdysoneless cell cycle regulator | 122882 | 166321 | 138286 | 45, 26, 9 |
| 10 | ENTPD1 | ectonucleoside triphosphate diphosphohydrolase 1 | 138185 | 119977 | 282889 | 29, 43, 32 |
| 10 | ENTPD7 | ectonucleoside triphosphate diphosphohydrolase 7 | 198018 | 155287 | 119929 | 13, 25, 38 |
| 10 | ELOVL3 | ELOVL fatty acid elongase 3 | 119915 | 166197 | 107859 | 35, 6, 32 |
| 10 | EMX2 | empty spiracles homeobox 2 | 170370 | 165650 | 107560 | 17, 44, 15 |
| 10 | EBLN1 | endogenous Bomavirus-like nucleoprotein 1 | 223601 | 136770 | 148444 | 38, |
| 10 | EPC1 | enhancer of polycomb homolog 1 | 120616 | 170759 | 216937 | |
| 10 | ENKUR | enkurin, TRPC channel interacting protein | 151023 | 099256 | 185875 | 42, 30, 38 |
| 10 | ENO4 | enolase family member 4 | 188316 | 165868 | 187164 | 38, 22, 37 |
| 10 | ECHDC3 | enoyl-CoA hydratase domain containing 3 | 134463 | 148429 | 148426 | 1, 9, 31 |
| 10 | ECHS1 | enoyl-CoA hydratase, short chain 1 | 127884 | 148803 | 148832 | 34, 41, 28 |
| 10 | ERLIN1 | ER lipid raft associated 1 | 107566 | 120054 | 213341 | 1, 3, 6 |
| 10 | ERCC6 | ERCC excision repair 6, chromatin remodeling factor | 225830 | 165606 | 243251 | 32, |
| 10 | EDRF1 | erythroid differentiation regulatory factor 1 | 107938 | 175018 | 154485 | |
| 10 | EIF3A | eukaryotic translation initiation factor 3 subunit A | 107581 | 188613 | 119979 | 40, 12, 19 |
| 10 | EIF4EBP2 | eukaryotic translation initiation factor 4E binding protein 2 | 148730 | 172731 | 156574 | 24, 22, 40 |
| 10 | EIF5AL1 | eukaryotic translation initiation factor 5A-like 1 | 253626 | 165424 | 185303 | 30, 21, 44 |
| 10 | EXOC6 | exocyst complex component 6 | 138190 | 152804 | 187553 | 42, 31, 43 |
| 10 | EXOSC1 | exosome component 1 | 171311 | 171314 | 171307 | 29, 30, 3 |
| 10 | FAM107B | family with sequence similarity 107 member B | 065809 | 151474 | 185267 | |
| 10 | FAM13C | family with sequence similarity 13 member C | 148541 | 165443 | 165449 | 26, 1, 14 |
| 10 | FAM149B1 | family with sequence similarity 149 member B1 | 138286 | 122882 | 213551 | 26, 2, 45 |
| 10 | FAM160B1 | family with sequence similarity 160 member B1 | 151553 | 099204 | 165832 | 1, 26, 11 |
| 10 | FAM170B | family with sequence similarity 170 member B | 172538 | 165633 | 204161 | 38, 8, |
| 10 | FAM171A1 | family with sequence similarity 171 member A1 | 148468 | 152465 | 077943 | 8, 36, 26 |
| 10 | FAM175B | family with sequence similarity 175 member B | 165660 | 203791 | 019995 | 43, 4, 45 |
| 10 | FAM188A | family with sequence similarity 188 member A | 148481 | 077943 | 165983 | 45, 8, 42 |
| 10 | FAM196A | family with sequence similarity 196 member A | 188916 | 150760 | 214285 | 28, 20, 44 |
| 10 | FAM204A | family with sequence similarity 204 member A | 165669 | 107560 | 119973 | 11, 30, 15 |
| 10 | FAM208B | family with sequence similarity 208 member B | 108021 | 196372 | 057608 | 22, 45, 19 |
| 10 | FAM21A | Family with sequence similarity 21 member A | 099290 | 204149 | 188611 | 29, 11, 21 |
| 10 | FAM21C | Family with sequence similarity 21 member C | 172661 | 172671 | 188234 | |
| 10 | FAM213A | family with sequence similarity 213 member A | 122378 | 133665 | 108219 | 1, 25, 43 |
| 10 | FAM24A | family with sequence similarity 24 member A | 203795 | 213185 | 119965 | 38, |
| 10 | FAM24B | family with sequence similarity 24 member B | 213185 | 138161 | 203795 | 31, 3, 19 |
| 10 | FAM25A | family with sequence similarity 25 member A | 188100 | 148671 | 148672 | 9, 5, 32 |
| 10 | FAM25C | family with sequence similarity 25 member C | 276430 | 264717 | 170324 | |
| 10 | FAM25G | family with sequence similarity 25 member G | 189090 | 265190 | 204172 | |
| 10 | FAM35A | family with sequence similarity 35 member A | 122376 | 148672 | 184923 | 1, 42, 31 |
| 10 | FAM45A | family with sequence similarity 45 member A | 119979 | 107581 | 183605 | 4, 3, 13 |
| 10 | FAM53B | family with sequence similarity 53 member B | 189319 | 258539 | 203791 | 22, 21, 43 |
| 10 | FAS | Fas cell surface death receptor | 026103 | 107796 | 138135 | 5, 14, 1 |
| 10 | FBXL15 | F-box and leucine rich repeat protein 15 | 107872 | 059915 | 107874 | 41, 31, 21 |
| 10 | FBXW4 | F-box and WD repeat domain containing 4 | 107829 | 166169 | 107831 | 29, 3, 18 |
| 10 | FBXO18 | F-box protein, helicase, 18 | 134452 | 134461 | 134470 | 1, 34, 28 |
| 10 | FRMPD2 | FERM and PDZ domain containing 2 | 170324 | 276430 | 107643 | 42, 14, 8 |
| 10 | FRMD4A | FERM domain containing 4A | 151474 | 282246 | 065809 | |
| 10 | FGF8 | fibroblast growth factor 8 | 107831 | 107829 | 107833 | 16, 12, |
| 10 | FGFBP3 | fibroblast growth factor binding protein 3 | 174721 | 107854 | 095564 | 12, 27, 8 |
| 10 | FGFR2 | fibroblast growth factor receptor 2 | 066468 | 120008 | 107669 | 11, 3, 29 |
| 10 | FANK1 | fibronectin type III and ankyrin repeat domains 1 | 203780 | 089876 | 148848 | 18, 38, 42 |
| 10 | FOXI2 | forkhead box I2 | 186766 | 214285 | 180745 | 17, 44, |
| 10 | FRA10AC1 | FRA10A associated CGG repeat 1 | 148690 | 095464 | 108231 | 11, 10, 31 |
| 10 | FRAT1 | FRAT1, WNT signaling pathway regulator | 165879 | 213390 | 181274 | 31, 34, 29 |
| 10 | FRAT2 | FRAT2, WNT signaling pathway regulator | 181274 | 165879 | 052749 | 26, 19, 3 |
| 10 | FFAR4 | free fatty acid receptor 4 | 186188 | 138180 | 138207 | 3, 16, 10 |
| 10 | FZD8 | frizzled class receptor 8 | 177283 | 177291 | 148513 | 14, 29, 9 |
| 10 | FRG2B | FSHD region gene 2 family member B | 225899 | 171772 | 096746 | |
| 10 | FUOM | fucose mutarotase | 148803 | 165828 | 127884 | 31, 36, 33 |
| 10 | FUT11 | fucosyltransferase 11 | 196968 | 176986 | 172586 | 34, 18, 29 |
| 10 | FXYD4 | FXYD domain containing ion transport regulator 4 | 150201 | 198915 | 169813 | 17, 33, 38 |
| 10 | GPRIN2 | G protein regulated inducer of neurite outgrowth 2 | 204175 | 204174 | 204176 | 28, 38, 16 |
| 10 | GPR158 | G protein-coupled receptor 158 | 151025 | 185875 | 095777 | 34, 8, 33 |
| 10 | GPR26 | G protein-coupled receptor 26 | 154478 | 154473 | 121898 | 44, 8, 17 |
| 10 | GRK5 | G protein-coupled receptor kinase 5 | 198873 | 165672 | 148908 | 21, 29, 36 |
| 10 | GJD4 | gap junction protein delta 4 | 177291 | 108100 | 177283 | 12, 8, |
| 10 | GATA3 | GATA binding protein 3 | 107485 | 165632 | 048740 | 11, 29, 4 |
| 10 | GFRA1 | GDNF family receptor alpha 1 | 151892 | 107518 | 182645 | 29, 23, 1 |
| 10 | GDI2 | GDP dissociation inhibitor 2 | 057608 | 108021 | 134461 | 41, 36, 45 |
| 10 | GAD2 | glutamate decarboxylase 2 | 136750 | 095777 | 077420 | 28, 8, 20 |
| 10 | GLUD1 | glutamate dehydrogenase 1 | 148672 | 188100 | 122376 | 18, 17, 26 |
| 10 | GRID1 | glutamate ionotropic receptor delta type subunit 1 | 182771 | 107771 | 062650 | 40, 37, 8 |
| 10 | GOT1 | glutamic-oxaloacetic transaminase 1 | 120053 | 119946 | 119919 | 15, 40, 29 |
| 10 | GLRX3 | glutaredoxin 3 | 108010 | 237489 | 176769 | 4, 9, 7 |
| 10 | GSTO1 | glutathione S-transferase omega 1 | 148834 | 197748 | 065621 | 43, 29, 34 |
| 10 | GSTO2 | glutathione S-transferase omega 2 | 065621 | 148834 | 148841 | 29, 18, 33 |
| 10 | GPAM | glycerol-3-phosphate acyltransferase, mitochondrial | 119927 | 150594 | 119913 | |
| 10 | GBF1 | golgi brefeldin A resistant guanine nucleotide exchange factor 1 | 107862 | 107859 | 077150 | 25, 21, 1 |
| 10 | GOLGA7B | golgin A7 family member B | 155265 | 120057 | 095713 | |
| 10 | GDF10 | growth differentiation factor 10 | 266524 | 204179 | 263761 | 11, 38, 18 |
| 10 | GDF2 | growth differentiation factor 2 | 263761 | 266524 | 265203 | 19, |
| 10 | GHITM | growth hormone inducible transmembrane protein | 165678 | 185737 | 188373 | 15, 7, 34 |
| 10 | GTPBP4 | GTP binding protein 4 | 107937 | 107929 | 148377 | 45, 3, 37 |
| 10 | H2AFY2 | H2A histone family member Y2 | 099284 | 197467 | 042286 | 12, 27, 14 |
| 10 | HMX2 | H6 family homeobox 2 | 188816 | 188620 | 154473 | 17, 33, 44 |
| 10 | HMX3 | H6 family homeobox 3 | 188620 | 196177 | 188816 | 27, |
| 10 | HSPA12A | heat shock protein family A (Hsp70) member 12A | 165868 | 165863 | 188316 | 26, 31, 2 |
| 10 | HSPA14 | heat shock protein family A (Hsp70) member 14 | 187522 | 185267 | 152455 | |
| 10 | HERC4 | HECT and RLD domain containing E3 ubiquitin protein ligase 4 | 148634 | 096717 | 138347 | 11, 42, 7 |
| 10 | HECTD2 | HECT domain E3 ubiquitin protein ligase 2 | 165338 | 180628 | 119938 | 11, 45, 33 |
| 10 | HELLS | helicase, lymphoid-specific | 119969 | 108239 | 108242 | 7, 11, 12 |
| 10 | HHEX | hematopoietically expressed homeobox | 152804 | 138160 | 138190 | 18, 11, 40 |
| 10 | HPSE2 | heparanase 2 (inactive) | 172987 | 107521 | 119946 | |
| 10 | HNRNPF | heterogeneous nuclear ribonucleoprotein F | 169813 | 150201 | 243660 | 34, 39, 4 |
| 10 | HNRNPH3 | heterogeneous nuclear ribonucleoprotein H3 | 096746 | 108187 | 204130 | 1, 36, 40 |
| 10 | HK1 | hexokinase 1 | 156515 | 156510 | 075073 | 40, 21, 6 |
| 10 | HKDC1 | hexokinase domain containing 1 | 156510 | 156502 | 156515 | 30, 39, 16 |
| 10 | HPS1 | HPS1, biogenesis of lysosomal organelles complex 3 subunit 1 | 107521 | 119943 | 172987 | 34, 26, 21 |
| 10 | HPS6 | HPS6, biogenesis of lysosomal organelles complex 2 subunit 3 | 166189 | 120029 | 198728 | 27, 26, 21 |
| 10 | HTRA1 | HtrA serine peptidase 1 | 166033 | 254636 | 187908 | 43, 31, 32 |
| 10 | HABP2 | hyaluronan binding protein 2 | 148702 | 148737 | 197893 | 19, 17, 37 |
| 10 | HIF1AN | hypoxia inducible factor 1 alpha subunit inhibitor | 166135 | 166136 | 075891 | 29, 21, 40 |
| 10 | IKZF5 | IKAROS family zinc finger 5 | 095574 | 179988 | 196177 | 26, 25, 45 |
| 10 | ITPRIP | inositol 1,4,5-trisphosphate receptor interacting protein | 148841 | 065621 | 120051 | 42, 24, 39 |
| 10 | IPMK | inositol polyphosphate multikinase | 151151 | 122952 | 122873 | 45, 4, 15 |
| 10 | INPP5A | inositol polyphosphate-5-phosphatase A | 068383 | 171813 | 148826 | 29, 18, 37 |
| 10 | INPP5F | inositol polyphosphate-5-phosphatase F | 198825 | 151929 | 197771 | 12, 26, 40 |
| 10 | IDE | insulin degrading enzyme | 119912 | 198060 | 138160 | 18, 22, 29 |
| 10 | ITGA8 | integrin subunit alpha 8 | 077943 | 148468 | 148481 | 45, 42, 33 |
| 10 | ITGB1 | integrin subunit beta 1 | 150093 | 150076 | 099250 | 45, 43, 25 |
| 10 | ITIH2 | inter-alpha-trypsin inhibitor heavy chain 2 | 151655 | 123243 | 151657 | 19, 36, 15 |
| 10 | ITIH5 | inter-alpha-trypsin inhibitor heavy chain family member 5 | 123243 | 198879 | 151655 | 32, 42, 1 |
| 10 | IFIT1 | interferon induced protein with tetratricopeptide repeats 1 | 185745 | 204010 | 152778 | 13, 4, 44 |
| 10 | IFIT1B | interferon induced protein with tetratricopeptide repeats 1B | 204010 | 119917 | 185745 | 7, 5, |
| 10 | IFIT2 | interferon induced protein with tetratricopeptide repeats 2 | 119922 | 107798 | 119917 | 11, 2, 5 |
| 10 | IFIT3 | interferon induced protein with tetratricopeptide repeats 3 | 119917 | 119922 | 204010 | 4, 5, 36 |
| 10 | IFIT5 | interferon induced protein with tetratricopeptide repeats 5 | 152778 | 185745 | 152779 | 38, 4, 37 |
| 10 | IL15RA | interleukin 15 receptor subunit alpha | 134470 | 134452 | 134460 | 3, 34, 24 |
| 10 | IL2RA | interleukin 2 receptor subunit alpha | 134460 | 134470 | 134453 | 39, 2, 5 |
| 10 | INA | internexin neuronal intermediate filament protein alpha | 148798 | 235376 | 156374 | 26, 14, 8 |
| 10 | IDI1 | isopentenyl-diphosphate delta isomerase 1 | 067064 | 148377 | 047056 | 34, 4, 2 |
| 10 | IDI2 | isopentenyl-diphosphate delta isomerase 2 | 148377 | 107937 | 067064 | 25, 10, 23 |
| 10 | JAKMIP3 | Janus kinase and microtubule interacting protein 3 | 188385 | 176171 | 151640 | 38, 8, 33 |
| 10 | JMJD1C | jumonji domain containing 1C | 171988 | 148572 | 165476 | 18, 22, 40 |
| 10 | KAZALD1 | Kazal type serine peptidase inhibitor domain 1 | 107821 | 107819 | 107807 | 6, 13, 3 |
| 10 | KIAA1217 | KIAA1217 | 120549 | 165312 | 107863 | 40, 24, 23 |
| 10 | KIAA1462 | KIAA1462 | 165757 | 197321 | 107951 | 40, 11, 10 |
| 10 | KIF1BP | KIF1 binding protein | 198954 | 165732 | 122862 | 45, 40, 42 |
| 10 | KLLN | killin, p53-regulated DNA replication inhibitor | 227268 | 138138 | 171862 | 22, 14, |
| 10 | KIN | Kin17 DNA and RNA binding protein | 151657 | 151655 | 165629 | 3, 5, 26 |
| 10 | KNDC1 | kinase non-catalytic C-lobe domain containing 1 | 171798 | 197177 | 171794 | 26, 31, 8 |
| 10 | KIF11 | kinesin family member 11 | 138160 | 119912 | 152804 | 22, 9, 2 |
| 10 | KIF20B | kinesin family member 20B | 138182 | 152782 | 148680 | 4, 13, 22 |
| 10 | KIF5B | kinesin family member 5B | 170759 | 165322 | 120616 | 11, 29, 35 |
| 10 | KLF6 | Kruppel like factor 6 | 067082 | 107959 | 165568 | 1, 30, 5 |
| 10 | LARP4B | La ribonucleoprotein domain family member 4B | 107929 | 180525 | 107937 | |
| 10 | LBX1 | ladybird homeobox 1 | 138136 | 107807 | 166167 | |
| 10 | LGI1 | leucine rich glioma inactivated 1 | 108231 | 148690 | 176273 | 8, 43, 26 |
| 10 | LRRC18 | leucine rich repeat containing 18 | 165383 | 283652 | 165633 | 9, 38, |
| 10 | LRRC20 | leucine rich repeat containing 20 | 172731 | 148734 | 148730 | 38, 25, 15 |
| 10 | LRRC27 | leucine rich repeat containing 27 | 148814 | 165752 | 171813 | 38, 39, 34 |
| 10 | LRRTM3 | leucine rich repeat transmembrane neuronal 3 | 198739 | 183230 | 108176 | 28, 8, 3 |
| 10 | LRIT1 | leucine rich repeat, Ig-like and transmembrane domains 1 | 148602 | 204033 | 148604 | 38, 25, 14 |
| 10 | LRIT2 | leucine rich repeat, Ig-like and transmembrane domains 2 | 204033 | 148600 | 148602 | 14, 8, |
| 10 | LZTS2 | leucine zipper tumor suppressor 2 | 107816 | 107815 | 186862 | 35, 2, 7 |
| 10 | LCOR | ligand dependent nuclear receptor corepressor | 196233 | 155629 | 187122 | 11, 2, 22 |
| 10 | LDB1 | LIM domain binding 1 | 198728 | 166189 | 148840 | 29, 4, 15 |
| 10 | LDB3 | LIM domain binding 3 | 122367 | 122375 | 107779 | 15, 25, 18 |
| 10 | LIPA | lipase A, lysosomal acid type | 107798 | 138135 | 119922 | 36, 5, 13 |
| 10 | LIPF | lipase F, gastric type | 182333 | 204022 | 204021 | 37, 13, 10 |
| 10 | LIPJ | lipase family member J | 204022 | 184719 | 182333 | 38, 10, |
| 10 | LIPK | lipase family member K | 204021 | 182333 | 204020 | |
| 10 | LIPM | lipase family member M | 173239 | 204020 | 152766 | |
| 10 | LIPN | lipase family member N | 204020 | 204021 | 173239 | 25, |
| 10 | LINC00959 | Long intergenic non-protein coding RNA 959 | 237489 | 108001 | 108010 | 2, 18, 22 |
| 10 | KAT6B | lysine acetyltransferase 6B | 156650 | 156110 | 188716 | 40, 23, 22 |
| 10 | LYZL1 | lysozyme like 1 | 120563 | 095739 | 197321 | 38, 10, |
| 10 | LYZL2 | lysozyme like 2 | 151033 | 107968 | 183621 | 38, 10, 23 |
| 10 | LOXL4 | lysyl oxidase like 4 | 138131 | 166024 | 119943 | 42, 35, 20 |
| 10 | MFSD13A | major facilitator superfamily domain containing 13A | 138111 | 120055 | 138107 | 4, 22, 18 |
| 10 | MALRD1 | MAM and LDL receptor class A domain containing 1 | 204740 | 165997 | 120594 | 10, 38, 23 |
| 10 | MBL2 | mannose binding lectin 2 | 165471 | 107984 | 150275 | 19, |
| 10 | MRC1 | mannose receptor C-type 1 | 260314 | 148483 | 148482 | 36, 1, 5 |
| 10 | MKI67 | marker of proliferation Ki-67 | 148773 | 132334 | 170430 | 22, 21, 3 |
| 10 | MARVELD1 | MARVEL domain containing 1 | 155254 | 119986 | 155256 | 6, 34, 35 |
| 10 | MMP21 | matrix metallopeptidase 21 | 154485 | 107938 | 188690 | |
| 10 | MXI1 | MAX interactor 1, dimerization protein | 119950 | 148700 | 119953 | 2, 11, 31 |
| 10 | MEIG1 | meiosis/spermiogenesis associated 1 | 197889 | 152457 | 152463 | |
| 10 | MARCH5 | membrane associated ring-CH-type finger 5 | 198060 | 107864 | 119912 | 39, 31, 42 |
| 10 | MARCH8 | membrane associated ring-CH-type finger 8 | 165406 | 012779 | 172671 | 7, 22, 1 |
| 10 | MPP7 | membrane palmitoylated protein 7 | 150054 | 169126 | 095787 | 4, 45, 38 |
| 10 | MGEA5 | meningioma expressed antigen 5 (hyaluronidase) | 198408 | 107833 | 120049 | 37, 18, 29 |
| 10 | MAT1A | methionine adenosyltransferase 1A | 151224 | 122359 | 170788 | 19, 21, 15 |
| 10 | MSRB2 | methionine sulfoxide reductase B2 | 148450 | 165309 | 168267 | 17, 30, 44 |
| 10 | METTL10 | methyltransferase like 10 | 203791 | 189319 | 165660 | 9, 17, 24 |
| 10 | MSMB | microseminoprotein beta | 263639 | 266412 | 274209 | 33, 25, 37 |
| 10 | MASTL | microtubule associated serine/threonine kinase like | 120539 | 136758 | 107897 | 3, 24, 6 |
| 10 | MCM10 | minichromosome maintenance 10 replication initiation factor | 065328 | 123240 | 165623 | 34, 21, 3 |
| 10 | MCMBP | minichromosome maintenance complex binding protein | 197771 | 198825 | 107651 | |
| 10 | MCU | mitochondrial calcium uniporter | 156026 | 107745 | 138315 | 18, 9, 24 |
| 10 | MICU1 | mitochondrial calcium uptake 1 | 107745 | 148719 | 156026 | 3, 17, 45 |
| 10 | MTPAP | mitochondrial poly(A) polymerase | 107951 | 165757 | 107968 | 3, 2, 24 |
| 10 | MRPL43 | mitochondrial ribosomal protein L43 | 055950 | 119906 | 095539 | 30, 26, 28 |
| 10 | MRPS16 | mitochondrial ribosomal protein S16 | 182180 | 213551 | 156042 | 41, 45, 43 |
| 10 | MTG1 | mitochondrial ribosome associated GTPase 1 | 148824 | 254536 | 203772 | |
| 10 | MAPK8 | mitogen-activated protein kinase 8 | 107643 | 170324 | 128805 | 9, 24, 45 |
| 10 | MAP3K8 | mitogen-activated protein kinase kinase kinase 8 | 107968 | 107951 | 151033 | 43, 35, 26 |
| 10 | MMS19 | MMS19 homolog, cytosolic iron-sulfur assembly component | 155229 | 171307 | 165886 | 43, 42, 21 |
| 10 | MKX | mohawk homeobox | 150051 | 099246 | 169126 | 11, 44, 27 |
| 10 | MORN4 | MORN repeat containing 4 | 171160 | 203942 | 155252 | 8, 18, 6 |
| 10 | MSS51 | MSS51 mitochondrial translational activator | 166343 | 138279 | 107758 | 15, 25, 23 |
| 10 | MTRNR2L5 | MT-RNR2-like 5 | 249860 | 150275 | 122952 | 38, |
| 10 | MTRNR2L7 | MT-RNR2-like 7 | 256892 | 148513 | 283179 | |
| 10 | MMRN2 | multimerin 2 | 173269 | 107779 | 173267 | 42, 32, 44 |
| 10 | MINPP1 | multiple inositol-polyphosphate phosphatase 1 | 107789 | 214562 | 198682 | 13, 19, 43 |
| 10 | MLLT10 | myeloid/lymphoid or mixed-lineage leukemia; translocated to, 10 | 078403 | 180592 | 136770 | 45, 26, 13 |
| 10 | MYOF | myoferlin | 138119 | 095596 | 138180 | 30, 23, 33 |
| 10 | MYPN | myopalladin | 138347 | 148634 | 179774 | 18, 9, 25 |
| 10 | MRLN | myoregulin | 227877 | 165449 | 108091 | |
| 10 | MYO3A | myosin IIIA | 095777 | 151025 | 136750 | 38, 28, 23 |
| 10 | MYOZ1 | myozenin 1 | 177791 | 166348 | 166317 | 25, 15, 10 |
| 10 | ASAH2 | N-acylsphingosine amidohydrolase 2 | 188611 | 099290 | 198964 | 4, 35, 38 |
| 10 | ASAH2B | N-acylsphingosine amidohydrolase 2B | 204147 | 198964 | 148584 | 42, 25, 38 |
| 10 | NDUFB8 | NADH:ubiquinone oxidoreductase subunit B8 | 166136 | 255339 | 166135 | 29, 3, 15 |
| 10 | NANOS1 | nanos C2HC-type zinc finger 1 | 188613 | 151893 | 107581 | 20, 3, 15 |
| 10 | NDST2 | N-deacetylase and N-sulfotransferase 2 | 166507 | 272916 | 148660 | 5, 1, 36 |
| 10 | NEBL | nebulette | 078114 | 120594 | 204683 | 15, 40, 26 |
| 10 | NRAP | nebulin related anchoring protein | 197893 | 148702 | 165806 | 25, 15, 18 |
| 10 | NEURL1 | neuralized E3 ubiquitin protein ligase 1 | 107954 | 183128 | 107957 | 28, 29, 14 |
| 10 | NRG3 | neuregulin 3 | 185737 | 178217 | 165678 | 8, 35, 25 |
| 10 | NET1 | neuroepithelial cell transforming 1 | 173848 | 178462 | 178372 | 42, 3, 24 |
| 10 | NEUROG3 | neurogenin 3 | 122859 | 099282 | 171224 | 16, |
| 10 | NPFFR1 | neuropeptide FF receptor 1 | 148734 | 180817 | 172731 | 38, |
| 10 | NPS | neuropeptide S | 214285 | 188916 | 186766 | |
| 10 | NPY4R | neuropeptide Y receptor Y4 | 204174 | 264230 | 204175 | 24, 16, |
| 10 | NRP1 | neuropilin 1 | 099250 | 150093 | 148498 | 11, 45, 43 |
| 10 | NHLRC2 | NHL repeat containing 2 | 196865 | 198924 | 043591 | |
| 10 | NKX1-2 | NK1 homeobox 2 | 229544 | 065154 | 107902 | 12, |
| 10 | NKX2-3 | NK2 homeobox 3 | 119919 | 120053 | 155287 | 16, 36, |
| 10 | NKX6-2 | NK6 homeobox 2 | 148826 | 068383 | 171811 | 8, 20, 17 |
| 10 | NMT2 | N-myristoyltransferase 2 | 152465 | 152464 | 148468 | 29, 6, 3 |
| 10 | NOC3L | NOC3 like DNA replication regulator | 173145 | 138193 | 108239 | 13, 6, 42 |
| 10 | NODAL | nodal growth differentiation factor | 156574 | 148730 | 107719 | 10, 12, 8 |
| 10 | NSUN6 | NOP2/Sun RNA methyltransferase family member 6 | 241058 | 165995 | 165997 | 24, 13, 15 |
| 10 | NSMCE4A | NSE4 homolog A, SMC5-SMC6 complex component | 107672 | 107669 | 138162 | 29, 22, 9 |
| 10 | NFKB2 | nuclear factor kappa B subunit 2 | 077150 | 107862 | 059915 | |
| 10 | NRBF2 | nuclear receptor binding factor 2 | 148572 | 122877 | 171988 | 30, 9, 22 |
| 10 | NCOA4 | nuclear receptor coactivator 4 | 266412 | 265354 | 263639 | |
| 10 | NOLC1 | nucleolar and coiled-body phosphoprotein 1 | 166197 | 148840 | 119915 | 9, 37, 23 |
| 10 | NPM3 | nucleophosmin/nucleoplasmin 3 | 107833 | 107831 | 198408 | 3, 21, 26 |
| 10 | NUDT13 | nudix hydrolase 13 | 166321 | 122884 | 122882 | 9, 7, 18 |
| 10 | NUDT5 | nudix hydrolase 5 | 165609 | 065665 | 151465 | 30, 35, 21 |
| 10 | NUTM2A | NUT family member 2A | 184923 | 122376 | 214562 | 28, 17, |
| 10 | NUTM2B | NUT family member 2B | 188199 | 122852 | 228570 | |
| 10 | NUTM2D | NUT family member 2D | 214562 | 184923 | 107789 | 15, 20, 38 |
| 10 | NUTM2E | NUT family member 2E | 228570 | 188199 | 133661 | |
| 10 | MGMT | O-6-methylguanine-DNA methyltransferase | 170430 | 148773 | 108001 | 27, 37, 21 |
| 10 | OLAH | oleoyl-ACP hydrolase | 152463 | 197889 | 176244 | 32, 38, 23 |
| 10 | OR13A1 | olfactory receptor family 13 subfamily A member 1 | 256574 | 165512 | 012779 | 38, |
| 10 | OPALIN | oligodendrocytic myelin paranodal and inner loop protein | 197430 | 107447 | 095587 | 8, 17, 39 |
| 10 | OIT3 | oncoprotein induced transcript 3 | 138315 | 156026 | 138308 | 19, 24, 36 |
| 10 | OPN4 | opsin 4 | 122375 | 062650 | 122367 | 14, 38, |
| 10 | OPTN | optineurin | 123240 | 151468 | 065328 | 25, 43, 6 |
| 10 | OAT | ornithine aminotransferase | 065154 | 182022 | 229544 | 13, 45, 2 |
| 10 | OTUD1 | OTU deubiquitinase 1 | 165312 | 179133 | 120549 | 24, 29, 40 |
| 10 | OGDHL | oxoglutarate dehydrogenase-like | 197444 | 178645 | 227345 | 8, 43, 26 |
| 10 | PAX2 | paired box 2 | 075891 | 166135 | 119906 | 17, 29, 27 |
| 10 | PITX3 | paired like homeodomain 3 | 107859 | 119915 | 107862 | 25, |
| 10 | PTF1A | pancreas specific transcription factor, 1a | 168267 | 148450 | 179133 | 28, |
| 10 | PNLIP | pancreatic lipase | 175535 | 203837 | 187021 | 28, 19, 1 |
| 10 | PNLIPRP1 | pancreatic lipase related protein 1 | 187021 | 175535 | 266200 | 4, 28, 19 |
| 10 | PNLIPRP2 | pancreatic lipase related protein 2 (gene/pseudegene) | 266200 | 187021 | 165863 | 28, 4, 16 |
| 10 | PNLIPRP3 | pancreatic lipase related protein 3 | 203837 | 182645 | 175535 | 18, 35, 30 |
| 10 | PANK1 | pantothenate kinase 1 | 152782 | 152779 | 138182 | 18, 34, 37 |
| 10 | PARD3 | par-3 family cell polarity regulator | 148498 | 099250 | 108094 | 18, 3, 24 |
| 10 | PTCHD3 | patched domain containing 3 | 182077 | 107897 | 099246 | 38, |
| 10 | PDLIM1 | PDZ and LIM domain 1 | 107438 | 173124 | 095637 | 1, 30, 41 |
| 10 | PDZD7 | PDZ domain containing 7 | 186862 | 107816 | 107819 | 1, 21, 17 |
| 10 | PDZD8 | PDZ domain containing 8 | 165650 | 165646 | 170370 | 9, 19, 37 |
| 10 | PPIF | peptidylprolyl isomerase F | 108179 | 108175 | 165424 | 21, 2, 3 |
| 10 | PRF1 | perforin 1 | 180644 | 107719 | 138316 | 36, 5, 39 |
| 10 | PRDX3 | peroxiredoxin 3 | 165672 | 183605 | 198873 | 4, 7, 34 |
| 10 | PPRC1 | peroxisome proliferator-activated receptpr gamma, coactivator-related 1 | 148840 | 198728 | 166197 | 18, 37, 40 |
| 10 | PBLD | phenazine biosynthesis like protein domain containing | 108187 | 179774 | 096746 | 17, 4, 19 |
| 10 | PTEN | phosphatase and tensin homolog | 171862 | 227268 | 184719 | 22, 11, 29 |
| 10 | PALD1 | phosphatase domain containing, paladin 1 | 107719 | 156574 | 180644 | 41, 26, 21 |
| 10 | PI4K2A | phosphatidylinositol 4-kinase type 2 alpha | 155252 | 171160 | 119986 | 38, 43, 4 |
| 10 | PIP4K2A | phosphatidylinositol-5-phosphate 4-kinase type 2 alpha | 150867 | 077327 | 165309 | 22, 35, 6 |
| 10 | PDE6C | phosphodiesterase 6C | 095464 | 138207 | 148690 | 10, 44, 14 |
| 10 | PFKP | phosphofructokinase, platelet | 067057 | 185736 | 107959 | 38, 40, 34 |
| 10 | PGAM1 | phosphoglycerate mutase 1 | 171314 | 052749 | 171311 | 21, 34, 13 |
| 10 | PIK3AP1 | phosphoinositide-3-kinase adapter protein 1 | 155629 | 077147 | 196233 | 22, 21, 7 |
| 10 | PLA2G12B | phospholipase A2 group XIIB | 138308 | 138315 | 122884 | 17, 19, 15 |
| 10 | PLCE1 | phospholipase C epsilon 1 | 138193 | 176273 | 173145 | 10, 42, 11 |
| 10 | PLPP4 | phospholipid phosphatase 4 | 203805 | 107651 | 120008 | 10, 6, 35 |
| 10 | LHPP | phospholysine phosphohistidine inorganic pyrophosphate phosphatase | 107902 | 229544 | 258539 | 33, 41, 21 |
| 10 | PRTFDC1 | phosphoribosyl transferase domain containing 1 | 099256 | 107863 | 151023 | 37, 45, 8 |
| 10 | PSTK | phosphoseryl-tRNA kinase | 179988 | 119965 | 095574 | 29, 38, 34 |
| 10 | PTER | phosphotriesterase related | 165983 | 148481 | 165985 | 4, 34, 9 |
| 10 | PHYH | phytanoyl-CoA 2-hydroxylase | 107537 | 165623 | 086475 | 26, 1, 29 |
| 10 | PHYHIPL | phytanoyl-CoA 2-hydroxylase interacting protein like | 165443 | 122870 | 148541 | 14, 8, 26 |
| 10 | PGBD3 | piggyBac transposable element derived 3 | 243251 | 225830 | 070748 | |
| 10 | PITRM1 | pitrilysin metallopeptidase 1 | 107959 | 067057 | 067082 | 42, 43, 24 |
| 10 | PLAC9 | placenta specific 9 | 189129 | 133678 | 122359 | 15, 32, 14 |
| 10 | PLAU | plasminogen activator, urokinase | 122861 | 148660 | 222047 | 40, 28, 37 |
| 10 | PSD | pleckstrin and Sec7 domain containing | 059915 | 077150 | 107872 | 31, 8, 26 |
| 10 | PLEKHA1 | pleckstrin homology domain containing A1 | 107679 | 138152 | 254636 | 1, 32, 29 |
| 10 | PLEKHS1 | pleckstrin homology domain containing S1 | 148735 | 165806 | 198924 | 42, 30, 23 |
| 10 | PLXDC2 | plexin domain containing 2 | 120594 | 204740 | 078114 | 26, 6, 15 |
| 10 | PARG | poly(ADP-ribose) glycohydrolase | 227345 | 197444 | 204152 | 13, 42, 39 |
| 10 | PAOX | polyamine oxidase | 148832 | 127884 | 254536 | 34, 27, 1 |
| 10 | PCGF5 | polycomb group ring finger 5 | 180628 | 148677 | 165338 | 22, 42, 11 |
| 10 | PCGF6 | polycomb group ring finger 6 | 156374 | 148798 | 148835 | 9, 6, 44 |
| 10 | PKD2L1 | polycystin 2 like 1, transient receptor potential cation channel | 107593 | 196072 | 099194 | 14, 10, 38 |
| 10 | KCNMA1 | potassium calcium-activated channel subfamily M alpha 1 | 156113 | 281042 | 151208 | 34, 10, 44 |
| 10 | KCNK18 | potassium two pore domain channel subfamily K member 18 | 186795 | 148704 | 165646 | |
| 10 | KCNIP2 | potassium voltage-gated channel interacting protein 2 | 120049 | 198408 | 120029 | 1, 15, 8 |
| 10 | PRPF18 | pre-mRNA processing factor 18 | 165630 | 165626 | 282246 | 30, 29, 6 |
| 10 | PDCD11 | programmed cell death 11 | 148843 | 173915 | 138172 | 21, 22, 1 |
| 10 | PDCD4 | programmed cell death 4 | 150593 | 203867 | 214413 | 36, 40, 42 |
| 10 | PRLHR | prolactin releasing hormone receptor | 119973 | 165669 | 151893 | 2, 44, 25 |
| 10 | PROSER2 | proline and serine rich 2 | 148426 | 134463 | 151461 | 44, 9, 6 |
| 10 | PRR26 | Proline rich 26 | 180525 | 151240 | 107929 | |
| 10 | PRAP1 | proline rich acidic protein 1 | 165828 | 130643 | 148803 | 13, 16, 19 |
| 10 | P4HA1 | prolyl 4-hydroxylase subunit alpha 1 | 122884 | 138308 | 166321 | 30, 45, 18 |
| 10 | PSAP | prosaposin | 197746 | 107738 | 122863 | 13, 40, 26 |
| 10 | PRKCQ | protein kinase C theta | 065675 | 170525 | 198879 | 39, 15, 40 |
| 10 | PRKG1 | protein kinase, cGMP-dependent, type I | 185532 | 148584 | 177613 | 15, 29, 45 |
| 10 | PPP1R3C | protein phosphatase 1 regulatory subunit 3C | 119938 | 165338 | 107854 | 15, 42, 24 |
| 10 | PPP2R2D | protein phosphatase 2 regulatory subunit Bdelta | 175470 | 176769 | 176171 | 7, 23, 34 |
| 10 | PPP3CB | protein phosphatase 3 catalytic subunit beta | 107758 | 166343 | 166348 | 1, 6, 29 |
| 10 | PTPN20 | protein tyrosine phosphatase, non-receptor type 20 | 204179 | 204176 | 266524 | |
| 10 | PTPRE | protein tyrosine phosphatase, receptor type E | 132334 | 180745 | 148773 | 18, 5, 45 |
| 10 | PCDH15 | protocadherin related 15 | 150275 | 165471 | 249860 | |
| 10 | PCBD1 | pterin-4 alpha-carbinolamine dehydratase 1 | 166228 | 166224 | 107731 | 43, 28, 27 |
| 10 | PWWP2B | PWWP domain containing 2B | 171813 | 148814 | 068383 | 36, 28, 26 |
| 10 | PYROXD2 | pyridine nucleotide-disulphide oxidoreductase domain 2 | 119943 | 138131 | 107521 | 9, 40, 27 |
| 10 | PPA1 | pyrophosphatase (inorganic) 1 | 180817 | 079332 | 148734 | 37, 28, 6 |
| 10 | R3HCC1L | R3H domain and coiled-coil containing 1 like | 166024 | 095713 | 138131 | 42, 31, 40 |
| 10 | RAB11FIP2 | RAB11 family interacting protein 2 | 107560 | 170370 | 165669 | 31, 29, 11 |
| 10 | RAB18 | RAB18, member RAS oncogene family | 099246 | 182077 | 150051 | 31, 13, 8 |
| 10 | RASSF4 | Ras association domain family member 4 | 107551 | 187783 | 165507 | 34, 40, 26 |
| 10 | RSU1 | Ras suppressor protein 1 | 148484 | 165985 | 107611 | 30, 7, 42 |
| 10 | RASGEF1A | RasGEF domain family member 1A | 198915 | 169826 | 150201 | 29, 38, 28 |
| 10 | REEP3 | receptor accessory protein 3 | 165476 | 171988 | 183230 | 29, 33, 40 |
| 10 | RGS10 | regulator of G-protein signaling 10 | 148908 | 198873 | 151923 | 22, 1, 15 |
| 10 | RNLS | renalase, FAD dependent amine oxidase | 184719 | 171862 | 204022 | 10, 1, 31 |
| 10 | RET | ret proto-oncogene | 165731 | 165733 | 169826 | 26, 23, 8 |
| 10 | RGR | retinal G protein coupled receptor | 148604 | 148602 | 107771 | 14, 25, 8 |
| 10 | RBP3 | retinol binding protein 3 | 265203 | 263761 | 265763 | 14, 25, 8 |
| 10 | RBP4 | retinol binding protein 4 | 138207 | 186188 | 095464 | 19, 28, 15 |
| 10 | ARHGAP12 | Rho GTPase activating protein 12 | 165322 | 148516 | 170759 | 1, 26, 23 |
| 10 | ARHGAP19 | Rho GTPase activating protein 19 | 213390 | 269891 | 165879 | 22, 34, 41 |
| 10 | ARHGAP21 | Rho GTPase activating protein 21 | 107863 | 120549 | 099256 | 11, 7, 18 |
| 10 | ARHGAP22 | Rho GTPase activating protein 22 | 128805 | 107643 | 128815 | 35, |
| 10 | RHOBTB1 | Rho related BTB domain containing 1 | 072422 | 170312 | 196932 | 43, 34, 26 |
| 10 | RTKN2 | rhotekin 2 | 182010 | 150347 | 138311 | 5, 18, 10 |
| 10 | RPP30 | ribonuclease P/MRP subunit p30 | 148688 | 148680 | 148677 | 31, 29, 7 |
| 10 | RPP38 | ribonuclease P/MRP subunit p38 | 152464 | 176244 | 152465 | 17, |
| 10 | RPS24 | ribosomal protein S24 | 138326 | 148606 | 108175 | 43, 33, 7 |
| 10 | RRP12 | ribosomal RNA processing 12 homolog | 052749 | 181274 | 171314 | 43, 21, 1 |
| 10 | RPEL1 | ribulose-5-phosphate-3-epimerase like 1 | 235376 | 076685 | 148798 | 1, 42, 3 |
| 10 | RBM17 | RNA binding motif protein 17 | 134453 | 134460 | 170525 | 22, 40, 18 |
| 10 | RBM20 | RNA binding motif protein 20 | 203867 | 108055 | 150593 | 29, 24, 6 |
| 10 | POLR3A | RNA polymerase III subunit A | 148606 | 151208 | 138326 | 3, 16, 40 |
| 10 | RUFY2 | RUN and FYVE domain containing 2 | 204130 | 096746 | 138346 | 26, 13, 6 |
| 10 | SFMBT2 | Scm-like with four mbt domains 2 | 198879 | 065675 | 123243 | 29, 26, 31 |
| 10 | SEC23IP | SEC23 interacting protein | 107651 | 197771 | 203805 | 18, 37, 42 |
| 10 | SEC24C | SEC24 homolog C, COPII coat complex component | 176986 | 172650 | 196968 | 18, 16, 5 |
| 10 | SEC31B | SEC31 homolog B, COPII coat complex component | 075826 | 075290 | 255339 | 39, 18, 27 |
| 10 | SEC61A2 | Sec61 translocon alpha 2 subunit | 065665 | 181192 | 165609 | 38, 8, 23 |
| 10 | SFRP5 | secreted frizzled related protein 5 | 120057 | 155256 | 155265 | 26, 14, 28 |
| 10 | SAR1A | secretion associated Ras related GTPase 1A | 079332 | 156521 | 180817 | 26, 15, 22 |
| 10 | SEPHS1 | selenophosphate synthetase 1 | 086475 | 107537 | 165626 | 12, 31, 6 |
| 10 | SEMA4G | semaphorin 4G | 095539 | 055950 | 107815 | 37, 16, 19 |
| 10 | SRGN | serglycin | 122862 | 198954 | 122958 | 45, 5, 7 |
| 10 | STK32C | serine/threonine kinase 32C | 165752 | 151640 | 148814 | 9, 3, 38 |
| 10 | SH2D4B | SH2 domain containing 4B | 178217 | 108219 | 185737 | 36, 38, 8 |
| 10 | SH3PXD2A | SH3 and PX domains 2A | 107957 | 107954 | 107960 | 9, 15, 29 |
| 10 | SPRN | shadow of prion protein homolog (zebrafish) | 203772 | 148824 | 130649 | 27, 40, 14 |
| 10 | SHOC2 | SHOC2, leucine rich repeat scaffold protein | 108061 | 214413 | 150594 | 22, 42, 25 |
| 10 | SHTN1 | shootin 1 | 187164 | 188316 | 283266 | 8, 42, 38 |
| 10 | SFXN2 | sideroflexin 2 | 156398 | 138175 | 166272 | 17, 16, |
| 10 | SFXN3 | sideroflexin 3 | 107819 | 186862 | 107821 | 45, 39, 40 |
| 10 | SFXN4 | sideroflexin 4 | 183605 | 119979 | 165672 | 27, 3, 7 |
| 10 | STAM | signal transducing adaptor molecule | 136738 | 165996 | 148483 | 13, 26, 39 |
| 10 | SIRT1 | sirtuin 1 | 096717 | 108176 | 148634 | 11, 22, 31 |
| 10 | SKIDA1 | SKI/DACH domain containing 1 | 180592 | 204682 | 078403 | 11, 25, 12 |
| 10 | SLIT1 | slit guidance ligand 1 | 187122 | 196233 | 269891 | |
| 10 | SLF2 | SMC5-SMC6 complex localization factcr 2 | 119906 | 075891 | 055950 | 18, 22, 41 |
| 10 | SLC16A12 | solute carrier family 16 member 12 | 152779 | 152778 | 152782 | 28, 2, 17 |
| 10 | SLC16A9 | solute carrier family 16 member 9 | 165449 | 148541 | 227877 | 29, 17, 42 |
| 10 | SLC18A2 | solute carrier family 18 member A2 | 165646 | 186795 | 165650 | 40, 2, 28 |
| 10 | SLC18A3 | solute carrier family 18 member A3 | 187714 | 070748 | 178645 | 19, 8, 14 |
| 10 | SLC25A16 | solute carrier family 25 member 16 | 122912 | 138346 | 138336 | 37, 1, 32 |
| 10 | SLC25A28 | solute carrier family 25 member 28 | 155287 | 119919 | 198018 | 34, 26, 6 |
| 10 | SLC29A3 | solute carrier family 29 member 3 | 198246 | 107731 | 107736 | 32, 30, 38 |
| 10 | SLC35G1 | solute carrier family 35 member G1 | 176273 | 108231 | 138193 | |
| 10 | SLC39A12 | solute carrier family 39 member 12 | 148482 | 260314 | 165995 | 8, 17, 14 |
| 10 | SORBS1 | sorbin and SH3 domain containing 1 | 095637 | 107438 | 059573 | 1, 26, 11 |
| 10 | SORCS1 | sortilin related VPS10 domain containing receptor 1 | 108018 | 156395 | 108039 | 11, 14, 25 |
| 10 | SORCS3 | sortilin related VPS10 domain containing receptor 3 | 156395 | 120051 | 108018 | 38, 25, 8 |
| 10 | SPOCK2 | SPARC/osteonectin, cwcv and kazal like domains proteoglycan 2 | 107742 | 122863 | 138303 | 26, 27, 8 |
| 10 | SPAG6 | sperm associated antigen 6 | 077327 | 168283 | 150867 | 38, 34, 31 |
| 10 | SGMS1 | sphingomyelin synthase 1 | 198964 | 188611 | 204147 | 42, 45, 11 |
| 10 | SGPL1 | sphingosine-1-phosphate lyase 1 | 166224 | 166220 | 166228 | 40, 29, 4 |
| 10 | ST8SIA6 | ST8 alpha-N-acetyl-neuraminide alpha-2,8-sialyltransferase 6 | 148488 | 026025 | 165996 | 5, 33, |
| 10 | STAMBPL1 | STAM binding protein like 1 | 138134 | 152766 | 107796 | 36, 13, 2 |
| 10 | SLK | STE20 like kinase | 065613 | 107960 | 065618 | 29, 9, 4 |
| 10 | SCD | stearoyl-CoA desaturase | 099194 | 107593 | 075290 | 1, 23, 21 |
| 10 | SAMD8 | sterile alpha motif domain containing 8 | 156671 | 079393 | 165637 | 18, 16, 4 |
| 10 | STN1 | STN1, CST complex subunit | 107960 | 107957 | 065613 | |
| 10 | STOX1 | storkhead box 1 | 165730 | 060339 | 107625 | 27, 40, 20 |
| 10 | SMC3 | structural maintenance of chromosomes 3 | 108055 | 138166 | 203867 | 31, 21, 1 |
| 10 | SUFU | SUFU negative regulator of hedgehog signaling | 107882 | 138107 | 171206 | 22, 25, 36 |
| 10 | SVIL | supervillin | 197321 | 120563 | 165757 | 18, 25, 15 |
| 10 | SUV39H2 | suppressor of variegation 3-9 homolog 2 | 152455 | 187522 | 152457 | 9, 45, 25 |
| 10 | SFTPA1 | surfactant protein A1 | 122852 | 185303 | 188199 | 20, 10, 33 |
| 10 | SFTPA2 | surfactant protein A2 | 185303 | 253626 | 122852 | 10, 20, 33 |
| 10 | SFTPD | surfactant protein D | 133661 | 228570 | 133678 | 20, 18, 10 |
| 10 | SMNDC1 | survival motor neuron domain containing 1 | 119953 | 119950 | 138166 | 1, 18, 32 |
| 10 | SUPV3L1 | Suv3 like RNA helicase | 156502 | 122958 | 156510 | 44, 9, 1 |
| 10 | SFR1 | SWI5 dependent homologous recombination repair protein 1 | 156384 | 065618 | 197748 | 38, 18, 7 |
| 10 | SYCE1 | synaptonemal complex central element protein 1 | 171772 | 130649 | 225899 | 38, 2, 42 |
| 10 | SYNPO2L | synaptopodin 2 like | 166317 | 177791 | 172650 | 15, 25, 18 |
| 10 | SYT15 | synaptotagmin 15 | 204176 | 204175 | 204179 | 42, 4, 33 |
| 10 | SNCG | synuclein gamma | 173267 | 173269 | 148671 | 26, 2, 27 |
| 10 | TACR2 | tachykinin receptpr 2 | 075073 | 156515 | 099282 | 25, 13, 16 |
| 10 | TNKS2 | tankyrase 2 | 107854 | 119938 | 174721 | 26, 2, 32 |
| 10 | TAF3 | TATA-box binding protein associated factor 3 | 165632 | 165629 | 107485 | |
| 10 | TAF5 | TATA-box binding protein associated factor 5 | 148835 | 156374 | 173915 | 22, 4, 3 |
| 10 | TBC1D12 | TBC1 domain family member 12 | 108239 | 173145 | 119969 | 40, 31, 37 |
| 10 | TLX1 | T-cell leukemia homeobox 1 | 107807 | 107821 | 138136 | 37, 16, 8 |
| 10 | TCTN3 | tectonic family member 3 | 119977 | 059573 | 138185 | 40, 29, 24 |
| 10 | TECTB | tectorin beta | 119913 | 119927 | 197142 | 14, |
| 10 | TEX36 | testis expressed 36 | 175018 | 175029 | 107938 | 32, 38, |
| 10 | TET1 | tet methylcytosine dioxygenase 1 | 138336 | 122912 | 060339 | 18, 12, 2 |
| 10 | TSPAN14 | tetraspanin 14 | 108219 | 122378 | 178217 | 35, 34, 27 |
| 10 | TSPAN15 | tetraspanin 15 | 099282 | 075073 | 122859 | 4, 28, 16 |
| 10 | THNSL1 | threonine synthase like 1 | 185875 | 151023 | 151025 | 3, 13, 28 |
| 10 | TBATA | thymus, brain and testes associated | 166220 | 138316 | 166224 | 39, 38, |
| 10 | TIAL1 | TIA1 cytotoxic granule associated RNA binding protein like 1 | 151923 | 148908 | 151929 | 37, 1, 18 |
| 10 | TLL2 | tolloid like 2 | 095587 | 197430 | 077147 | 17, 15, 28 |
| 10 | TCERG1L | transcription elongaticn regulator 1 like | 176769 | 108010 | 175470 | 20, 14, 40 |
| 10 | TCF7L2 | transcription factor 7 like 2 | 148737 | 151532 | 148702 | 11, 18, 15 |
| 10 | TFAM | transcription factor A, mitochondrial | 108064 | 072401 | 122870 | 13, 5, 22 |
| 10 | TACC2 | transforming acidic coiled-coil containing protein 2 | 138162 | 107672 | 138152 | 24, 32, 37 |
| 10 | TIMM23 | translocase of inner mitochondrial membrane 23 | 265354 | 188234 | 266412 | |
| 10 | TIMM23B | translocase of inner mitochondrial membrane 23 homolog B | 204152 | 227345 | 204149 | 34, 2, 26 |
| 10 | TM9SF3 | transmembrane 9 superfamily member 3 | 077147 | 095587 | 155629 | 40, 29, 16 |
| 10 | TMEM236 | transmembrane protein 236 | 148483 | 136738 | 260314 | 32, 16, 14 |
| 10 | TMEM254 | transmembrane protein 254 | 133678 | 133661 | 189129 | 13, 29, 2 |
| 10 | TMEM26 | transmembrane protein 26 | 196932 | 072422 | 183346 | 36, 34, 44 |
| 10 | TMEM72 | transmembrane protein 72 | 187783 | 107562 | 107551 | 2, 14, 17 |
| 10 | TRIM8 | tripartite motif containing 8 | 171206 | 107882 | 138175 | 29, 17, 20 |
| 10 | TRDMT1 | tRNA aspartic acid methyltransferase 1 | 107614 | 107611 | 026025 | 18, 7, 42 |
| 10 | TRUB1 | TruB pseudouridine synthase family member 1 | 165832 | 151553 | 107518 | 42, 22, 29 |
| 10 | TYSND1 | trypsin domain containing 1 | 156521 | 042286 | 079332 | 27, 7, 29 |
| 10 | TUBAL3 | tubulin alpha like 3 | 178462 | 178473 | 173848 | 18, 16, 24 |
| 10 | TUBB8 | tubulin beta 8 class VIII | 261456 | 096746 | 015171 | 35, 12, 38 |
| 10 | TUBGCP2 | tubulin gamma complex associated protein 2 | 130640 | 151651 | 198546 | 29, 27, 14 |
| 10 | TDRD1 | tudor domain containing 1 | 095627 | 165813 | 165816 | 38, 42, 33 |
| 10 | TWNK | twinkle mtDNA helicase | 107815 | 095539 | 107816 | |
| 10 | UBE2D1 | ubiquitin conjugating enzyme E2 D1 | 072401 | 122873 | 108064 | 43, 10, 25 |
| 10 | UBTD1 | ubiquitin domain containing 1 | 165886 | 155229 | 165887 | 29, 1, 34 |
| 10 | USP54 | ubiquitin specific peptidase 54 | 166348 | 107758 | 177791 | 18, 40, 33 |
| 10 | UNC5B | unc-5 netrin receptor B | 107731 | 166228 | 198246 | 6, 36, 2 |
| 10 | UTF1 | undifferentiated embryonic cell transcription factor 1 | 171794 | 171798 | 151650 | |
| 10 | UPF2 | UPF2 regulator of nonsense transcripts homolog (yeast) | 151461 | 148426 | 181192 | 34, 41, 2 |
| 10 | UCMA | upper zone of growth plate and cartilage matrix associated | 165623 | 065328 | 107537 | 11, 10, 8 |
| 10 | USMG5 | up-regulated during skeletal muscle growth 5 homolog (mouse) | 173915 | 148835 | 148843 | 43, 33, 19 |
| 10 | UCN3 | urocortin 3 | 178473 | 198610 | 178462 | 28, |
| 10 | UROS | uroporphyrinogen III synthase | 188690 | 154485 | 107949 | 9, 34, 26 |
| 10 | USP6NL | USP6 N-terminal like | 148429 | 048740 | 134463 | 22, 23, 44 |
| 10 | VENTX | VENT homeobox | 151650 | 171794 | 151651 | 29, 12, 14 |
| 10 | VAX1 | ventral anterior homeobox 1 | 148704 | 283266 | 186795 | 14, 38, 8 |
| 10 | VTI1A | vesicle transport through interaction with t-SNAREs 1A | 151532 | 023041 | 148737 | 34, 39, 7 |
| 10 | VIM | vimentin | 026025 | 107614 | 148488 | 11, 43, 6 |
| 10 | VCL | vinculin | 035403 | 222047 | 185009 | 42, 24, 44 |
| 10 | VDAC2 | voltage dependent anion channel 2 | 165637 | 156671 | 165644 | 1, 37, 25 |
| 10 | VWA2 | von Willebrand factor A domain containing 2 | 165816 | 095627 | 169129 | 42, 24, 18 |
| 10 | VPS26A | VPS26, retromer complex component A | 122958 | 122862 | 156502 | 45, 4, 29 |
| 10 | VSTM4 | V-set and transmembrane domain containing 4 | 165633 | 165383 | 172538 | 29, 11, 2 |
| 10 | WAPL | WAPL cohesin release factor | 062650 | 182771 | 122375 | 22, 18, 31 |
| 10 | WDR11 | WD repeat domain 11 | 120008 | 203805 | 066468 | 42, 40, 22 |
| 10 | WDR37 | WD repeat domain 37 | 047056 | 067064 | 185736 | 24, 42, 1 |
| 10 | WDFY4 | WDFY family member 4 | 128815 | 128805 | 283652 | 21, 41, 22 |
| 10 | WDFY4 | WDFY family member 4 | 283652 | 128815 | 165383 | 21, 41, 22 |
| 10 | WNT8B | Wnt family member 8B | 075290 | 099194 | 075826 | 42, 27, 12 |
| 10 | WBP1L | WW domain binding protein 1-like | 166272 | 156398 | 148795 | 43, 26, 35 |
| 10 | WAC | WW domain containing adaptor with coiled-coil | 095787 | 150054 | 095739 | 5, 4, 2 |
| 10 | XPNPEP1 | X-prolyl aminopeptidase 1 | 108039 | 108018 | 148700 | 37, 3, 34 |
| 10 | YME1L1 | YME1 like 1 ATPase | 136758 | 107890 | 120539 | |
| 10 | ZFAND4 | zinc finger AN1-type containing 4 | 172671 | 165406 | 172661 | 38, 22, 26 |
| 10 | ZCCHC24 | zinc finger CCHC-type containing 24 | 165424 | 108179 | 253626 | 26, 6, 8 |
| 10 | ZDHHC16 | zinc finger DHHC-type containing 16 | 171307 | 171311 | 155229 | 9, 24, 27 |
| 10 | ZDHHC6 | zinc finger DHHC-type containing 6 | 023041 | 197142 | 151532 | 36, 39, 16 |
| 10 | ZEB1 | zinc finger E-box binding homeobox 1 | 148516 | 183621 | 165322 | 25, 45, 42 |
| 10 | ZFYVE27 | zinc finger FYVE-type containing 27 | 155256 | 155254 | 120057 | 39, 13, 43 |
| 10 | ZMIZ1 | zinc finger MIZ-type containing 1 | 108175 | 138326 | 108179 | 40, 23, 12 |
| 10 | ZMYND11 | zinc finger MYND-type containing 11 | 015171 | 261456 | 151240 | 11, 29, 44 |
| 10 | ZNF22 | zinc finger protein 22 | 165512 | 165507 | 256574 | 22, 30, 11 |
| 10 | ZNF239 | zinc finger protein 239 | 196793 | 243660 | 198298 | 9, 36, 28 |
| 10 | ZNF248 | zinc finger protein 248 | 198105 | 283179 | 175395 | 31, 6, 39 |
| 10 | ZNF25 | zinc finger protein 25 | 175395 | 198105 | 189180 | 31, 29, 8 |
| 10 | ZNF32 | zinc finger protein 32 | 169740 | 198298 | 277288 | 29, 11, 34 |
| 10 | ZNF33A | zinc finger protein 33A | 189180 | 175395 | 075407 | 42, 36, 25 |
| 10 | ZNF33B | zinc finger protein 33B | 196693 | 075407 | 165733 | 4, 36, 17 |
| 10 | ZNF365 | zinc finger protein 365 | 138311 | 182010 | 181915 | 8, 29, 10 |
| 10 | ZNF37A | zinc finger protein 37A | 075407 | 189180 | 196693 | 29, 26, 31 |
| 10 | ZNF438 | zinc finger protein 438 | 183621 | 151033 | 148516 | |
| 10 | ZNF485 | zinc finger protein 485 | 198298 | 196793 | 169740 | 13, 30, 40 |
| 10 | ZNF487 | Zinc finger protein 487 | 243660 | 169813 | 196793 | |
| 10 | ZNF488 | zinc finger protein 488 | 265763 | 265203 | 265190 | 8, 42, 25 |
| 10 | ZNF503 | zinc finger protein 503 | 165655 | 165644 | 148655 | 27, 11, 28 |
| 10 | ZNF511 | zinc finger protein 511 | 198546 | 130640 | 283496 | 41, 16, 32 |
| 10 | ZNF518A | zinc finger protein 518A | 177853 | 107443 | 095585 | 42, 24, 39 |
| 10 | ZRANB1 | zinc finger RANBP2-type containing 1 | 019995 | 165660 | 175029 | 26, 14, 40 |
| 10 | ZSWIM8 | zinc finger SWIM-type containing 8 | 214655 | 172586 | 272916 | 22, 31, 28 |
| 10 | ZNF511-PRAP1 | ZNF511-PRAP1 readthrough | 283496 | 198546 | 130643 | |
| 10 | ZWINT | ZW10 interacting kinetochore protein | 122952 | 249860 | 151151 | 21, 12, 9 |
| 11 | ACCS | 1-aminocyclopropane-1-carboxylate synthase homolog (inactive) | 110455 | 205126 | 151348 | 26, 44, 2 |
| 11 | ACCSL | 1-aminocyclopropane-1-carboxylate synthase homolog (inactive) like | 205126 | 187479 | 110455 | |
| 11 | HTR3A | 5-hydroxytryptamine receptor 3A | 166736 | 149305 | 109906 | 41, 18, 20 |
| 11 | HTR3B | 5-hydroxytryptamine receptor 3B | 149305 | 048028 | 166736 | 16, 8, |
| 11 | PTS | 6-pyruvoyltetrahydropterin synthase | 150787 | 197580 | 188771 | 17, 1, 28 |
| 11 | DHCR7 | 7-dehydrocholesterol reductase | 172893 | 162105 | 172890 | 2, 21, 26 |
| 11 | ACAT1 | acetyl-CoA acetyltransferase 1 | 075239 | 166266 | 149308 | 1, 25, 18 |
| 11 | ASCL2 | achaete-scute family bHLH transcription factor 2 | 183734 | 180176 | 110665 | 16, 32, 28 |
| 11 | ASCL3 | achaete-scute family bHLH transcription factor 3 | 176009 | 176029 | 175348 | 10, |
| 11 | ACP2 | acid phosphatase 2, lysosomal | 134575 | 134574 | 025434 | 2, 36, 13 |
| 11 | ACRV1 | acrosomal vesicle protein 1 | 134940 | 149554 | 171053 | 38, 32, 8 |
| 11 | ACTN3 | actinin alpha 3 (gene/pseudogene) | 248746 | 174165 | 174080 | 15, 25, 12 |
| 11 | ACAD8 | acyl-CoA dehydrogenase family member 8 | 151498 | 151500 | 166105 | 39, 42, 8 |
| 11 | ADAMTS15 | ADAM metallopeptidase with thrombospondin type 1 motif 15 | 166106 | 134917 | 120451 | 15, 27, 42 |
| 11 | ADAMTS8 | ADAM metallopeptidase with thrombospondin type 1 motif 8 | 134917 | 196323 | 166106 | 20, 33, 27 |
| 11 | AP2A2 | adaptor related protein complex 2 alpha 2 subunit | 183020 | 177830 | 184956 | 26, 38, 37 |
| 11 | AP5B1 | adaptor related protein complex 5 beta 1 subunit | 254470 | 172922 | 172818 | 5, 1, 21 |
| 11 | AMPD3 | adenosine monophosphate deaminase 3 | 133805 | 148926 | 255823 | |
| 11 | AAMDC | adipogenesis associated Mth938 domain containing | 087884 | 048649 | 149262 | 2, 43, 31 |
| 11 | ARFGAP2 | ADP ribosylation factor GTPase activating protein 2 | 149182 | 149179 | 165912 | 44, 18, 36 |
| 11 | ARFIP2 | ADP ribosylation factor interacting protein 2 | 132254 | 110171 | 132286 | 21, 31, 9 |
| 11 | ARL14EP | ADP ribosylation factor like GTPase 14 effector protein | 152219 | 131808 | 066382 | 9, 7, 42 |
| 11 | ARL2 | ADP ribosylation factor like GTPase 2 | 213465 | 168062 | 273003 | 29, 16, 12 |
| 11 | ART1 | ADP-ribosyltransferase 1 | 129744 | 167311 | 129749 | 25, |
| 11 | ART5 | ADP-ribosyltransferase 5 | 167311 | 005801 | 129744 | 11, 38, 12 |
| 11 | ADM | adrenomedullin | 148926 | 133812 | 133805 | 1, 32, 29 |
| 11 | AHNAK | AHNAK nucleoprotein | 124942 | 149021 | 254772 | 18, 1, 42 |
| 11 | AKIP1 | A-kinase interacting protein 1 | 166452 | 166444 | 176029 | 13, 29, 3 |
| 11 | ALDH3B1 | aldehyde dehydrogenase 3 family member B1 | 006534 | 110057 | 110717 | 34, 9, 20 |
| 11 | ALDH3B2 | aldehyde dehydrogenase 3 family member B2 | 132746 | 132744 | 110057 | 24, 4, 23 |
| 11 | ALG8 | ALG8, alpha-1,3-glucosyltransferase | 159063 | 151366 | 188997 | 34, 4, 10 |
| 11 | ALG9 | ALG9, alpha-1,2-mannosyltransferase | 086848 | 137713 | 258529 | 21, 40, 9 |
| 11 | ACER3 | alkaline ceramidase 3 | 078124 | 182704 | 198488 | 13, 31, 29 |
| 11 | ALKBH3 | alkB homolog 3, alpha-ketoglutaratedependent dioxygenase | 166199 | 149084 | 187479 | 43, 13, 36 |
| 11 | ALKBH8 | alkB homolog 8, tRNA methyltransferase | 137760 | 152404 | 110675 | 45, 42, 3 |
| 11 | ALX4 | ALX homeobox 4 | 052850 | 151348 | 085117 | 10, 14, 8 |
| 11 | ACY3 | aminoacylase 3 | 132744 | 167800 | 132746 | 17, 3, 21 |
| 11 | AASDHPPT | aminoadipate-semialdehyde dehydrogenase-phosphopantetheinyl transferase | 149313 | 182359 | 152402 | 34, 7, 8 |
| 11 | APLP2 | amyloid beta precursor like protein 2 | 084234 | 170325 | 149418 | 42, 24, 45 |
| 11 | APBB1 | amyloid beta precursor protein binding family B member 1 | 166313 | 166311 | 110169 | 26, 31, 8 |
| 11 | ANAPC15 | anaphase promoting complex subunit 15 | 110200 | 149357 | 110203 | 7, 31, 41 |
| 11 | AMOTL1 | angiomotin like 1 | 166025 | 134627 | 150316 | 25, 2, 42 |
| 11 | ANGPTL5 | angiopoietin like 5 | 187151 | 137672 | 110318 | 15, 10, 35 |
| 11 | ABTB2 | ankyrin repeat and BTB domain containing 2 | 166016 | 135372 | 121691 | 6, 26, 31 |
| 11 | ANKK1 | ankyrin repeat and kinase domain containing 1 | 170209 | 149292 | 149295 | 38, |
| 11 | ANKRD13D | ankyrin repeat domain 13D | 172932 | 173020 | 172830 | 39, 31, 36 |
| 11 | ANKRD42 | ankyrin repeat domain 42 | 137494 | 165494 | 137500 | 42, 1, 36 |
| 11 | ANKRD49 | ankyrin repeat domain 49 | 168876 | 020922 | 257057 | 39, 43, 19 |
| 11 | ANO1 | anoctamin 1 | 131620 | 186895 | 168040 | 30, 29, 18 |
| 11 | ANO3 | anoctamin 3 | 134343 | 187398 | 169550 | 8, 10, 32 |
| 11 | ANO5 | anoctamin 5 | 171714 | 165973 | 091664 | 29, 25, 11 |
| 11 | ANO9 | anoctamin 9 | 185101 | 185187 | 174915 | 39, 31, 16 |
| 11 | APIP | APAF1 interacting protein | 149089 | 135373 | 110435 | 3, 34, 43 |
| 11 | APLNR | apelin receptor | 134817 | 183908 | 149115 | 36, 26, 32 |
| 11 | APOA1 | apolipoprotein A1 | 118137 | 110245 | 160584 | 19, 36, 38 |
| 11 | APOA4 | apolipoprotein A4 | 110244 | 110243 | 110245 | 19, 16, 5 |
| 11 | APOA5 | apolipoprotein A5 | 110243 | 109917 | 110244 | 19, 20, |
| 11 | APOC3 | apolipoprotein C3 | 110245 | 110244 | 118137 | 19, 36, 17 |
| 11 | API5 | apoptosis inhibitor 5 | 166181 | 148948 | 052841 | 42, 24, 39 |
| 11 | AQP11 | aquaporin 11 | 178301 | 074201 | 048649 | 29, 10, 2 |
| 11 | ARCN1 | archain 1 | 095139 | 118096 | 019144 | 26, 29, 24 |
| 11 | ARAP1 | ArfGAP with RhoGAP domain, ankyrin repeat and PH domain 1 | 186635 | 186642 | 214530 | 41, 22, 5 |
| 11 | ARL2-SNX15 | ARL2-SNX15 readthrough (NMD candidate) | 273003 | 213465 | 110025 | 9, 1, 43 |
| 11 | ARRB1 | arrestin beta 1 | 137486 | 261594 | 149273 | 43, 1, 16 |
| 11 | AIP | aryl hydrocarbon receptor interacting protein | 110711 | 172663 | 110697 | 29, 6, 26 |
| 11 | ARNTL | aryl hydrocarbon receptor nuclear translocator like | 133794 | 189431 | 148925 | 24, 39, 36 |
| 11 | ASRGL1 | asparaginase like 1 | 162174 | 197745 | 149021 | 41, 9, 27 |
| 11 | NARS2 | asparaginyl-tRNA synthetase 2, mitochondrial (putative) | 137513 | 033327 | 149256 | 11, 13, 18 |
| 11 | ATL3 | atlastin GTPase 3 | 184743 | 176485 | 133318 | 1, 11, 9 |
| 11 | ATM | ATM serine/threonine kinase | 149311 | 149308 | 166323 | 22, 30, 23 |
| 11 | ABCC8 | ATP binding cassette subfamily C member 8 | 006071 | 187486 | 006611 | 28, 20, 25 |
| 11 | ABCG4 | ATP binding cassette subfamily G member 4 | 172350 | 172273 | 160703 | 8, 39, 14 |
| 11 | ATP5L | ATP synthase, H+ transporting, mitochondrial Fo complex subunit G | 167283 | 110344 | 118058 | 7, 34, 4 |
| 11 | AGBL2 | ATP/GTP binding protein like 2 | 165923 | 109919 | 109920 | 42, 38, 39 |
| 11 | AMBRA1 | autophagy and beclin 1 regulator 1 | 110497 | 180720 | 180423 | 43, 26, 42 |
| 11 | ATG13 | autophagy related 13 | 175224 | 180423 | 175220 | 42, 43, 39 |
| 11 | ATG16L2 | autophagy related 16 like 2 | 168010 | 214530 | 137478 | 18, 37, 5 |
| 11 | ATG2A | autophagy related 2A | 110046 | 110047 | 068971 | 1, 21, 9 |
| 11 | BIRC2 | baculoviral IAP repeat containing 2 | 110330 | 023445 | 152558 | 10, 29, 4 |
| 11 | BIRC3 | baculoviral IAP repeat containing 3 | 023445 | 137693 | 110330 | 22, 26, 16 |
| 11 | BBS1 | Bardet-Biedl syndrome 1 | 174483 | 256349 | 174165 | 42, 29, 24 |
| 11 | BANF1 | barrier to autointegration factor 1 | 175334 | 175376 | 175315 | 29, 7, 16 |
| 11 | BARX2 | BARX homeobox 2 | 043039 | 134909 | 151715 | 4, 23, 16 |
| 11 | BATF2 | basic leucine zipper ATF-like transcription factor 2 | 168062 | 168070 | 213465 | 1, 16, 7 |
| 11 | BCL9L | B-cell CLL/lymphoma 9-like | 186174 | 160683 | 110375 | 12, |
| 11 | BAD | BCL2 associated agonist of cell death | 002330 | 149782 | 173264 | 34, 14, 26 |
| 11 | BEST1 | bestrophin 1 | 167995 | 167994 | 167996 | 14, 1, 5 |
| 11 | BET1L | Bet1 golgi vesicular membrane trafficking protein like | 177951 | 134954 | 188076 | 39, 42, 31 |
| 11 | B3GAT1 | beta-1,3-glucuronyltransferase 1 | 109956 | 149328 | 134954 | 26, 14, 8 |
| 11 | B3GAT3 | beta-1,3-glucuronyltransferase 3 | 149541 | 149489 | 089597 | 41, 9, 26 |
| 11 | B4GAT1 | beta-1,4-glucuronyltransferase 1 | 174684 | 174744 | 174669 | 8, 11, 17 |
| 11 | B4GALNT4 | beta-1,4-N-acetyl-galactosaminyltransferase 4 | 182272 | 142089 | 184363 | 27, 12, 14 |
| 11 | BC02 | beta-carotene oxygenase 2 | 197580 | 150783 | 150787 | 2, 15, 19 |
| 11 | BACE1 | beta-secretase 1 | 186318 | 167257 | 110274 | 34, 29, 6 |
| 11 | BUD | BH3-like motif containing, cell death inducer | 259571 | 137642 | 154127 | |
| 11 | BRSK2 | BR serine/threonine kinase 2 | 174672 | 078902 | 182208 | 14, 8, 40 |
| 11 | BDNF | brain derived neurotrophic factor | 176697 | 148943 | 121621 | 10, 2, 15 |
| 11 | BSX | brain specific homeobox | 188909 | 109944 | 109971 | 27, |
| 11 | BRMS1 | breast cancer metastasis suppressor 1 | 174744 | 174791 | 174684 | 18, 41, 34 |
| 11 | BSCL2 | BSCL2, seipin lipid droplet biogenesis associated | 168000 | 177363 | 162188 | 31, 8, 38 |
| 11 | BTBD10 | BTB domain containing 10 | 148925 | 133794 | 152266 | 31, 13, 8 |
| 11 | BTBD18 | BTB domain containing 18 | 233436 | 254732 | 198561 | 40, 24, 12 |
| 11 | BTG4 | BTG anti-proliferation factor 4 | 137707 | 110777 | 183644 | |
| 11 | BUD13 | BUD13 homolog | 137656 | 182985 | 109917 | 43, 9, 1 |
| 11 | C1QTNF4 | C1q and tumor necrosis factor related protein 4 | 172247 | 196666 | 109919 | 31, 28, 14 |
| 11 | C1QTNF5 | C1q and tumor necrosis factor related protein 5 | 223953 | 173456 | 235718 | 14, 34, 28 |
| 11 | C2CD3 | C2 calcium dependent domain containing 3 | 168014 | 175564 | 214517 | 16, 22, 18 |
| 11 | C2CD2L | C2CD2 like | 172375 | 172269 | 172273 | 10, 8, 38 |
| 11 | CDHR5 | cadherin related family member 5 | 099834 | 185507 | 070031 | 16, 17, 37 |
| 11 | CALCA | calcitonin related polypeptide alpha | 110680 | 175868 | 188487 | 40, 33, |
| 11 | CALCB | calcitonin related polypeptide beta | 175868 | 186104 | 110680 | 6, 8, 38 |
| 11 | CABP2 | calcium binding protein 2 | 167791 | 167797 | 084207 | 14, |
| 11 | CABP4 | calcium binding protein 4 | 175544 | 175514 | 172663 | |
| 11 | CRACR2B | calcium release activated channel regulator 2B | 177685 | 177666 | 177697 | 26, 42, 3 |
| 11 | CAPN1 | calpain 1 | 014216 | 204710 | 014138 | |
| 11 | CAPN5 | calpain 5 | 149260 | 198488 | 254550 | 1, 26, 31 |
| 11 | CREB3L1 | cAMP responsive element binding protein 3 like 1 | 157613 | 135365 | 149091 | 16, 37, 20 |
| 11 | CHST1 | carbohydrate sulfotransferase 1 | 175264 | 019505 | 181830 | 31, 34, 29 |
| 11 | CLCF1 | cardiotrophin-like cytokine factor 1 | 175505 | 256514 | 172531 | 37, 26, 6 |
| 11 | CPT1A | carnitine palmitoyltransferase 1A | 110090 | 132749 | 197345 | 5, 16, 42 |
| 11 | CARNS1 | carnosine synthase 1 | 172508 | 175463 | 175634 | 40, 8, 25 |
| 11 | CSNK2A3 | casein kinase 2 alpha 3 | 254598 | 110328 | 170242 | |
| 11 | CASP1 | caspase 1 | 137752 | 137757 | 204397 | 13, 34, 30 |
| 11 | CASP12 | caspase 12 (gene/pseudogene) | 204403 | 170967 | 196954 | 10, 15, |
| 11 | CASP4 | caspase 4 | 196954 | 204403 | 137757 | 6, 15, 19 |
| 11 | CASP5 | caspase 5 | 137757 | 196954 | 137752 | 15, 10, 16 |
| 11 | CARD16 | caspase recruitment domain family member 16 | 204397 | 137752 | 255221 | 9, 19, 16 |
| 11 | CARD17 | caspase recruitment domain family member 17 | 255221 | 204397 | 255501 | 32, |
| 11 | CARD18 | caspase recruitment domain family member 18 | 255501 | 255221 | 152578 | 15, 32, 10 |
| 11 | CAT | catalase | 121691 | 166016 | 135374 | 7, 19, 5 |
| 11 | CTNND1 | catenin delta 1 | 198561 | 233436 | 186509 | 24, 42, 4 |
| 11 | CTSC | cathepsin C | 109861 | 123892 | 168959 | 34, 5, 13 |
| 11 | CTSD | cathepsin D | 117984 | 250644 | 149043 | 34, 1, 13 |
| 11 | CTSF | cathepsin F | 174080 | 248746 | 182791 | 26, 1, 2 |
| 11 | CTSW | cathepsin W | 172543 | 172638 | 172500 | 5, 36, 18 |
| 11 | CATSPER1 | cation channel sperm associated 1 | 175294 | 175315 | 175229 | 38, 6, 20 |
| 11 | CBL | Cbl proto-oncogene | 110395 | 248712 | 076706 | 22, 11, 36 |
| 11 | CD151 | CD151 molecule (Raph blood group) | 177697 | 177685 | 177700 | 1, 3, 26 |
| 11 | CD248 | CD248 molecule | 174807 | 179292 | 174791 | 6, 1, 32 |
| 11 | CD3D | CD3d molecule | 167286 | 198851 | 160654 | 10, 39, 5 |
| 11 | CD3E | CD3e molecule | 198851 | 149573 | 167286 | 39, 5, 36 |
| 11 | CD3G | CD3g molecule | 160654 | 167286 | 110344 | 39, 5, 36 |
| 11 | CD44 | CD44 molecule (Indian blood group) | 026508 | 110435 | 110436 | 7, 5, 23 |
| 11 | CD5 | CD5 molecule | 110448 | 013725 | 167987 | 39, 36, 5 |
| 11 | CD59 | CD59 molecule | 085063 | 110427 | 110429 | |
| 11 | CD6 | CD6 molecule | 013725 | 110446 | 110448 | 39, 36, 5 |
| 11 | CD81 | CD81 molecule | 110651 | 064201 | 184281 | 26, 41, 1 |
| 11 | CD82 | CD82 molecule | 085117 | 052850 | 157570 | 41, 16, 1 |
| 11 | CDC42BPG | CDC42 binding protein kinase gamma | 171219 | 133895 | 110047 | 18, 26, 35 |
| 11 | CDC42EP2 | CDC42 effector protein 2 | 149798 | 014138 | 133884 | 1, 40, 13 |
| 11 | CDON | cell adhesion associated, oncogene regulated | 064309 | 109832 | 165526 | 1, 15, 29 |
| 11 | CADM1 | cell adhesion molecule 1 | 182985 | 204361 | 137656 | 11, 28, 26 |
| 11 | CAPRIN1 | cell cycle associated protein 1 | 135387 | 135363 | 135372 | 42, 38, 24 |
| 11 | CEND1 | cell cycle exit and neuronal differentiation 1 | 184524 | 177225 | 177542 | 26, 8, 14 |
| 11 | CDCA5 | cell division cycle associated 5 | 146670 | 168060 | 162300 | 21, 5, 18 |
| 11 | CEP126 | centrosomal protein 126 | 110318 | 187151 | 137691 | 31, 42, 28 |
| 11 | CEP164 | centrosomal protein 164 | 110274 | 186318 | 177103 | 43, 7, 13 |
| 11 | CEP295 | centrosomal protein 295 | 166004 | 166002 | 166012 | 19, 18, 22 |
| 11 | CEP57 | centrosomal protein 57 | 166037 | 077458 | 087053 | 9, 22, 13 |
| 11 | CHEK1 | checkpoint kinase 1 | 149554 | 134910 | 134940 | 9, 39, 45 |
| 11 | CHID1 | chitinase domain containing 1 | 177830 | 214063 | 183020 | 1, 2, 36 |
| 11 | CLNS1A | chloride nucleotide-sensitive channel 1A | 074201 | 149269 | 178301 | 18, 27, 3 |
| 11 | CCKBR | cholecystokinin B receptor | 110148 | 132259 | 170955 | 37, 8, 38 |
| 11 | CHKA | choline kinase alpha | 110721 | 110719 | 110066 | 38, 22, 12 |
| 11 | CHRM1 | cholinergic receptor muscarinic 1 | 168539 | 168003 | 197901 | 8, 33, 23 |
| 11 | CHRM4 | cholinergic receptor muscarinic 4 | 180720 | 110492 | 110497 | 36, 28, 14 |
| 11 | CHRNA10 | cholinergic receptor nicotinic alpha 10 subunit | 129749 | 129744 | 110713 | 22, 39, 27 |
| 11 | CHRDL2 | chordin like 2 | 054938 | 077514 | 166439 | 44, 33, 9 |
| 11 | C11orf1 | chromosome 11 open reading frame 1 | 137720 | 255561 | 109846 | 29, 45, 20 |
| 11 | C11orf16 | chromosome 11 open reading frame 16 | 176029 | 166452 | 176009 | 42, 20, 44 |
| 11 | C11orf21 | chromosome 11 open reading frame 21 | 110665 | 183734 | 064201 | 5, 36, 27 |
| 11 | C11orf24 | chromosome 11 open reading frame 24 | 171067 | 110066 | 162337 | |
| 11 | C11orf40 | chromosome 11 open reading frame 40 | 171987 | 197790 | 226288 | |
| 11 | C11orf42 | chromosome 11 open reading frame 42 | 180878 | 175485 | 051009 | 4, 38, 8 |
| 11 | C11orf45 | chromosome 11 open reading frame 45 | 174370 | 120457 | 120471 | 24, 42, 6 |
| 11 | C11orf49 | chromosome 11 open reading frame 49 | 149179 | 134569 | 149182 | 26, 1, 34 |
| 11 | C11orf52 | chromosome 11 open reading frame 52 | 149300 | 254445 | 150764 | 30, 26, 14 |
| 11 | C11orf53 | chromosome 11 open reading frame 53 | 150750 | 137727 | 214290 | 33, 20, |
| 11 | C11orf54 | chromosome 11 open reading frame 54 | 182919 | 166012 | 042429 | 17, 1, 7 |
| 11 | C11orf57 | chromosome 11 open reading frame 57 | 150776 | 150773 | 150779 | 9, 29, 4 |
| 11 | C11orf58 | chromosome 11 open reading frame 58 | 110696 | 110693 | 166689 | 7, 34, 30 |
| 11 | C11orf63 | chromosome 11 open reading frame 63 | 109944 | 109943 | 188909 | 1, 38, 29 |
| 11 | C11orf65 | chromosome 11 open reading frame 65 | 166323 | 149311 | 178202 | 38, 39, |
| 11 | C11orf68 | chromosome 11 open reading frame 68 | 175573 | 175592 | 175550 | 34, 14, 6 |
| 11 | C11orf70 | chromosome 11 open reading frame 70 | 137691 | 110318 | 137693 | 38, 30, 6 |
| 11 | C11orf71 | chromosome 11 open reading frame 71 | 180425 | 166741 | 282682 | 38, 1, 29 |
| 11 | C11orf71 | chromosome 11 open reading frame 71 | 282682 | 180425 | 076053 | 38, 1, 29 |
| 11 | C11orf74 | chromosome 11 open reading frame 74 | 166352 | 175097 | 148948 | 43, 29, 15 |
| 11 | C11orf80 | chromosome 11 open reading frame 80 | 173715 | 173898 | 173653 | 29, 26, 2 |
| 11 | C11orf84 | chromosome 11 open reading frame 84 | 168005 | 188070 | 072518 | 26, 9, 6 |
| 11 | C11orf86 | chromosome 11 open reading frame 86 | 173237 | 173621 | 173227 | 3, 37, 16 |
| 11 | C11orf87 | chromosome 11 open reading frame 87 | 185742 | 178105 | 149289 | 11, 8, 7 |
| 11 | C11orf88 | chromosome 11 open reading frame 88 | 183644 | 137707 | 204381 | 30, 10, 38 |
| 11 | C11orf91 | chromosome 11 open reading frame 91 | 205177 | 110427 | 110429 | |
| 11 | C11orf94 | chromosome 11 open reading frame 94 | 234776 | 121653 | 121680 | 38, |
| 11 | C11orf95 | chromosome 11 open reading frame 95 | 188070 | 133318 | 168005 | 1, 17, 12 |
| 11 | C11orf96 | chromosome 11 open reading frame 96 | 187479 | 166199 | 205126 | 17, 20, 11 |
| 11 | C11orf97 | Chromosome 11 open reading frame 97 | 257057 | 168876 | 134627 | |
| 11 | C11orf98 | chromosome 11 open reading frame 98 | 278615 | 255432 | 162194 | |
| 11 | CNTF | ciliary neurotrophic factor | 242689 | 255073 | 149124 | 22, 18, 11 |
| 11 | CLDN25 | claudin 25 | 228607 | 086827 | 048028 | |
| 11 | CLP1 | cleavage and polyadenylation factor I subunit 1 | 172409 | 166793 | 156599 | 1, 6, 13 |
| 11 | CPSF7 | cleavage and polyadenylation specific factor 7 | 149532 | 187049 | 256591 | 42, 9, 24 |
| 11 | CSTF3 | cleavage stimulation factor subunit 3 | 176102 | 280550 | 110422 | |
| 11 | CLPB | ClpB homolog, mitochondrial AAA ATPase chaperonin | 162129 | 165462 | 186642 | 38, 5, 21 |
| 11 | F2 | coagulation factor II, thrombin | 180210 | 175213 | 175216 | 19, 23, 15 |
| 11 | COPB1 | coatomer protein complex subunit beta 1 | 129083 | 133818 | 256206 | |
| 11 | CFL1 | cofilin 1 | 172757 | 172818 | 172803 | 41, 34, 21 |
| 11 | CCDC15 | coiled-coil domain containing 15 | 149548 | 221932 | 134955 | 11, 31, 29 |
| 11 | CCDC153 | coiled-coil domain containing 153 | 248712 | 172367 | 110395 | 20, 44, 38 |
| 11 | CCDC179 | coiled-coil domain containing 179 | 255359 | 198168 | 187398 | 10, 38, |
| 11 | CCDC34 | coiled-coil domain containing 34 | 109881 | 129151 | 205213 | 40, 37, 9 |
| 11 | CCDC73 | coiled-coil domain containing 73 | 186714 | 149100 | 135378 | 10, 38, 22 |
| 11 | CCDC81 | coiled-coil domain containing 81 | 149201 | 149196 | 151376 | 43, 30, 38 |
| 11 | CCDC82 | coiled-coil domain containing 82 | 149231 | 184384 | 183340 | 4, 22, 45 |
| 11 | CCDC83 | coiled-coil domain containing 83 | 150676 | 137501 | 073921 | 38, 8, |
| 11 | CCDC84 | coiled-coil domain containing 84 | 186166 | 176302 | 118181 | 20, 14, 44 |
| 11 | CCDC85B | coiled-coil domain containing 85B | 175602 | 172500 | 175592 | 41, 20, 6 |
| 11 | CCDC86 | coiled-coil domain containing 86 | 110104 | 172689 | 183134 | 29, 9, 38 |
| 11 | CCDC87 | coiled-coil domain containing 87 | 182791 | 174080 | 173992 | 38, 24, 29 |
| 11 | CCDC88B | coiled-coil domain containing 88B | 168071 | 126432 | 162302 | 5, 21, 7 |
| 11 | CCDC89 | coiled-coil domain containing 89 | 179071 | 137504 | 137501 | 38, 35, 6 |
| 11 | CCDC90B | coiled-coil domain containing 90B | 137500 | 137494 | 150672 | 4, 34, 30 |
| 11 | COLCA2 | colorectal cancer associated 2 | 214290 | 150750 | 110777 | 3, 23, 16 |
| 11 | COMMD9 | COMM domain containing 9 | 110442 | 179241 | 135362 | |
| 11 | CNTN5 | contactin 5 | 149972 | 183340 | 165895 | 10, 32, 14 |
| 11 | CCS | copper chaperone for superoxide dismutase | 173992 | 182791 | 239306 | 4, 19, 26 |
| 11 | CNIH2 | cornichon family AMPA receptor auxiliary protein 2 | 174871 | 174903 | 174851 | 28, 12, 8 |
| 11 | CORO1B | coronin 1B | 172725 | 175634 | 175514 | 21, 37, 9 |
| 11 | CTTN | cortactin | 085733 | 131626 | 162105 | 18, 30, 35 |
| 11 | CREBZF | CREB/ATF bZIP transcription factor | 137504 | 171202 | 179071 | 22, 16, 11 |
| 11 | CRY2 | cryptochrome circadian clock 2 | 121671 | 181830 | 121653 | 3, 42, 24 |
| 11 | CRYAB | crystallin alpha B | 109846 | 137720 | 170276 | 14, 25, 15 |
| 11 | CTR9 | CTR9 homolog, Paf1/RNA polymerase II complex component | 198730 | 072952 | 110321 | 18, 31, 26 |
| 11 | CELF1 | CUGBP, Elav-like family member 1 | 149187 | 165917 | 213619 | 3, 22, 21 |
| 11 | CUL5 | cullin 5 | 166266 | 179331 | 075239 | |
| 11 | CWC15 | CWC15 spliceosome associated protein homolog | 150316 | 166025 | 186280 | 29, 11, 15 |
| 11 | CWF19L2 | CWF19-like 2, cell cycle control (S. pombe) | 152404 | 152402 | 137760 | 24, 42, 40 |
| 11 | CLMP | CXADR like membrane protein | 166250 | 109971 | 023171 | 1, 45, 10 |
| 11 | CXCR5 | C-X-C motif chemokine receptor 5 | 160683 | 110367 | 186174 | 36, 22, 5 |
| 11 | CNGA4 | cyclic nucleotide gated channel alpha 4 | 132259 | 051009 | 110148 | 42, 38, 20 |
| 11 | CCND1 | cyclin D1 | 110092 | 172927 | 149716 | 18, 40, 3 |
| 11 | CDK2AP2 | cyclin dependent kinase 2 associated protein 2 | 167797 | 110697 | 167791 | 41, 9, 21 |
| 11 | CDKN1C | cyclin dependent kinase inhibitor 1C | 129757 | 053918 | 254827 | 32, 15, 28 |
| 11 | CST6 | cystatin E/M | 175315 | 175334 | 175294 | 15, 22, 40 |
| 11 | CSRP3 | cysteine and glycine rich protein 3 | 129170 | 177054 | 129173 | 15, 25, 18 |
| 11 | CHORDC1 | cysteine and histidine rich domain containing 1 | 110172 | 077616 | 165323 | 13, 11, 7 |
| 11 | CARS | cysteinyl-tRNA synthetase | 110619 | 205531 | 021762 | 21, 17, 3 |
| 11 | CYB5R2 | cytochrome b5 reductase 2 | 166394 | 166387 | 183378 | 38, 3, 34 |
| 11 | CYB561A3 | cytochrome b561 family member A3 | 162144 | 149476 | 149483 | 41, 36, 2 |
| 11 | COA4 | cytochrome c oxidase assembly factor 4 homolog | 181924 | 175581 | 175575 | 9, 29, 41 |
| 11 | COX8A | cytochrome c oxidase subunit 8A | 176340 | 110583 | 256100 | 15, 25, 12 |
| 11 | CYP2R1 | cytochrome P450 family 2 subfamily R member 1 | 186104 | 152270 | 175868 | 37, 38, 45 |
| 11 | CKAP5 | cytoskeleton associated protein 5 | 175216 | 180210 | 134569 | |
| 11 | CRTAM | cytotoxic and regulatory T-cell molecule | 109943 | 154127 | 109944 | 5, 8, 36 |
| 11 | DCHS1 | dachsous cadherin-related 1 | 166341 | 166340 | 158042 | 1, 26, 2 |
| 11 | DDB1 | damage specific DNA binding protein 1 | 167986 | 167992 | 149476 | 43, 21, 32 |
| 11 | DDB2 | damage specific DNA binding protein 2 | 134574 | 165912 | 134575 | 41, 9, 43 |
| 11 | DDX10 | DEAD-box helicase 10 | 178105 | 110723 | 185742 | 21, 6, 10 |
| 11 | DDX25 | DEAD-box helicase 25 | 109832 | 110060 | 064309 | |
| 11 | DDX6 | DEAD-box helicase 6 | 110367 | 118094 | 160683 | 13, 29, 41 |
| 11 | DEAF1 | DEAF1, transcription factor | 177030 | 069696 | 177106 | 4, 26, 34 |
| 11 | DCPS | decapping enzyme, scavenger | 110063 | 150455 | 110080 | 7, 41, 37 |
| 11 | DCUN1D5 | defective in cullin neddylation 1 domain containing 5 | 137692 | 137745 | 187240 | 13, 11, 35 |
| 11 | DEFB108B | defensin beta 108B | 184276 | 158483 | 254469 | 38, |
| 11 | DTX4 | deltex E3 ubiquitin ligase 4 | 110042 | 166801 | 197629 | 15, 40, 26 |
| 11 | DENND5A | DENN domain containing 5A | 184014 | 175356 | 166471 | 34, 7, 25 |
| 11 | DEPDC7 | DEP domain containing 7 | 121690 | 060749 | 176148 | 22, 19, 38 |
| 11 | DEUP1 | deuterosome assembly protein 1 | 165325 | 180773 | 166002 | 34, 38, 20 |
| 11 | DBX1 | developing brain homeobox 1 | 109851 | 166833 | 109854 | |
| 11 | DGKZ | diacylglycerol kinase zeta | 149091 | 157613 | 110492 | 3, 39, 22 |
| 11 | DAGLA | diacylglycerol lipase alpha | 134780 | 011347 | 124920 | 28, 37, 36 |
| 11 | DGAT2 | diacylglycerol O-acyltransferase 2 | 062282 | 166391 | 198382 | 1, 35, 38 |
| 11 | DKK3 | dickkopf WNT signaling pathway inhibitor 3 | 050165 | 170242 | 133816 | 8, 28, 26 |
| 11 | DLAT | dihydrolipoamide S-acetyltransferase | 150768 | 150764 | 150773 | 42, 43, 22 |
| 11 | DPP3 | dipeptidyl peptidase 3 | 254986 | 174516 | 256349 | 24, 36, 9 |
| 11 | DLG2 | discs large MAGUK scaffold protein 2 | 150672 | 137500 | 171204 | 8, 17, 14 |
| 11 | DIXDC1 | DIX domain containing 1 | 150764 | 149300 | 150768 | 6, 42, 8 |
| 11 | DDIAS | DNA damage induced apoptosis suppressor | 165490 | 137509 | 137502 | 9, 38, 11 |
| 11 | DDI1 | DNA damage inducible 1 homolog 1 | 170967 | 170962 | 204403 | 38, 8, |
| 11 | POLA2 | DNA polymerase alpha 2, accessory subunit | 014138 | 014216 | 149798 | 13, 39, 38 |
| 11 | POLD3 | DNA polymerase delta 3, accessory subunit | 077514 | 175536 | 054938 | 43, 22, 30 |
| 11 | POLD4 | DNA polymerase delta 4, accessory subunit | 175482 | 172613 | 256514 | 36, 23, 25 |
| 11 | DNAJB13 | DnaJ heat shock protein family (Hsp40) member B13 | 187726 | 175575 | 175567 | 42, 38, 44 |
| 11 | DNAJC24 | DnaJ heat shock protein family (Hsp40) member C24 | 170946 | 170959 | 148950 | 23, 11, 7 |
| 11 | DNAJC4 | DnaJ heat shock protein family (Hsp40) member C4 | 110011 | 149761 | 173511 | 29, 22, 20 |
| 11 | DPAGT1 | dolichyl-phosphate N-acetylglucosaminephosphotransferase 1 | 172269 | 188486 | 172375 | 39, 13, 30 |
| 11 | DRD2 | dopamine receptor D2 | 149295 | 170209 | 166682 | 26, 31, 20 |
| 11 | DRD4 | dopamine receptor D4 | 069696 | 070031 | 177030 | 14, 17, 6 |
| 11 | DPF2 | double PHD fingers 2 | 133884 | 149798 | 173825 | 34, 40, 1 |
| 11 | DCDC1 | doublecortin domain containing 1 | 170959 | 066382 | 170946 | |
| 11 | DRAP1 | DR1 associated protein 1 | 175550 | 175573 | 175513 | 28, 6, 22 |
| 11 | DSCAML1 | DS cell adhesion molecule like 1 | 177103 | 110274 | 137731 | 31, 14, 42 |
| 11 | DUSP8 | dual specificity phosphatase 8 | 184545 | 182208 | 278919 | 28, 14, 35 |
| 11 | DYNC2H1 | dynein cytoplasmic 2 heavy chain 1 | 187240 | 137692 | 170962 | 18, 1, 10 |
| 11 | DNHD1 | dynein heavy chain domain 1 | 179532 | 132286 | 132275 | 38, 36, 39 |
| 11 | E2F8 | E2F transcription factor 8 | 129173 | 129170 | 166833 | 39, 22, 24 |
| 11 | ELF5 | E74 like ETS transcription factor 5 | 135374 | 121691 | 135373 | 42, 34, 37 |
| 11 | EML3 | echinoderm microtubule associated protein like 3 | 149499 | 149480 | 149489 | 9, 41, 21 |
| 11 | EFEMP2 | EGF containing fibulin like extracellular matrix protein 2 | 172638 | 172732 | 172543 | 34, 6, 32 |
| 11 | EHBP1L1 | EH domain binding protein 1 like 1 | 173442 | 176973 | 173338 | 26, 6, 9 |
| 11 | EHD1 | EH domain containing 1 | 110047 | 171219 | 110046 | 36, 39, 42 |
| 11 | EI24 | EI24, autophagy associated transmembrane protein | 149547 | 149557 | 134910 | 4, 9, 40 |
| 11 | ELMOD1 | ELMO domain containing 1 | 110675 | 137760 | 170290 | |
| 11 | ELP4 | elongator acetyltransferase complex subunit 4 | 109911 | 148950 | 007372 | 10, 11, 17 |
| 11 | EED | embryonic ectoderm development | 074266 | 073921 | 149196 | 30, 26, 11 |
| 11 | EMSY | EMSY, BRCA2 interacting transcriptional repressor | 158636 | 179240 | 137507 | 9, 32, 42 |
| 11 | ENDOD1 | endonuclease domain containing 1 | 149218 | 263465 | 149212 | 31, 33, 2 |
| 11 | ESAM | endothelial cell adhesion molecule | 149564 | 019102 | 120458 | 32, 1, 15 |
| 11 | EPS8L2 | EPS8 like 2 | 177106 | 177030 | 177042 | 13, 27, 17 |
| 11 | ESRRA | estrogen related receptor alpha | 173153 | 219435 | 173113 | 3, 41, 9 |
| 11 | EHF | ETS homologous factor | 135373 | 135374 | 149089 | 42, 24, 3 |
| 11 | ETS1 | ETS proto-oncogene 1, transcription factor | 134954 | 149571 | 151702 | 22, 1, 26 |
| 11 | EEF1G | eukaryotic translation elongation factor 1 gamma | 254772 | 124942 | 255508 | |
| 11 | EIF1AD | eukaryotic translation initiation factor 1A domain containing | 175376 | 175467 | 175334 | 20, 27, 35 |
| 11 | EIF3F | eukaryotic translation initiation factor 3 subunit F | 175390 | 182261 | 166402 | 22, 21, 41 |
| 11 | EIF3M | eukaryotic translation initiation factor 3 subunit M | 149100 | 184937 | 186714 | 34, 7, 4 |
| 11 | EIF4G2 | eukaryotic translation initiation factor 4 gamma 2 | 110321 | 198730 | 236287 | 42, 45, 13 |
| 11 | EXPH5 | exophilin 5 | 110723 | 178202 | 178105 | 32, 42, 4 |
| 11 | EXT2 | exostosin glycosyltransferase 2 | 151348 | 110455 | 052850 | 7, 9, 2 |
| 11 | FOXRED1 | FAD dependent oxidoreductase domain containing 1 | 110074 | 182934 | 150455 | 9, 19, 39 |
| 11 | FAM111A | family with sequence similarity 111 member A | 166801 | 189057 | 110042 | 29, 24, 22 |
| 11 | FAM111B | family with sequence similarity 111 member B | 189057 | 255151 | 166801 | 22, 4, 39 |
| 11 | FAM118B | family with sequence similarity 118 member B | 197798 | 165526 | 182934 | 37, 7, 34 |
| 11 | FAM160A2 | family with sequence similarity 160 member A2 | 051009 | 180878 | 132259 | 42, 38, 39 |
| 11 | FAM168A | family with sequence similarity 168 member A | 054965 | 054967 | 021300 | 9, 27, 18 |
| 11 | FAM180B | family with sequence similarity 180 member B | 196666 | 123444 | 172247 | 26, |
| 11 | FAM181B | family with sequence similarity 181 member B | 182103 | 149256 | 137509 | 8, 40, 27 |
| 11 | FAM76B | family with sequence similarity 76 member B | 077458 | 149212 | 166037 | 22, 38, 40 |
| 11 | FAM86C1 | family with sequence similarity 86 member C1 | 158483 | 204571 | 184276 | 3, 6, 14 |
| 11 | FAM89B | family with sequence similarity 89 member B | 176973 | 173465 | 173442 | 34, 28, 41 |
| 11 | FANCF | Fanconi anemia complementation group F | 183161 | 091664 | 148935 | 39, 36, 21 |
| 11 | FADD | Fas associated via death domain | 168040 | 131620 | 131626 | 34, 29, 9 |
| 11 | FEZ1 | fasciculation and elongation protein zeta 1 | 149557 | 165495 | 149547 | 29, 8, 26 |
| 11 | FAT3 | FAT atypical cadherin 3 | 165323 | 110172 | 134640 | 12, 8, 33 |
| 11 | FADS1 | fatty acid desaturase 1 | 149485 | 134824 | 221968 | |
| 11 | FADS2 | fatty acid desaturase 2 | 134824 | 168496 | 149485 | 35, 33, 12 |
| 11 | FADS3 | fatty acid desaturase 3 | 221968 | 149485 | 167994 | 41, 26, 22 |
| 11 | FAR1 | fatty acyl-CoA reductase 1 | 197601 | 152266 | 262655 | 37, 26, 22 |
| 11 | FAU | FAU, ubiquitin like and ribosomal protein S30 fusion | 149806 | 174276 | 162298 | 43, 33, 31 |
| 11 | FBXO3 | F-box protein 3 | 110429 | 085063 | 135363 | 42, 4, 24 |
| 11 | FCHSD2 | FCH and double SH3 domains 2 | 137478 | 168010 | 175591 | 22, 31, 40 |
| 11 | FRMD8 | FERM domain containing 8 | 126391 | 162241 | 279576 | 24, 42, 4 |
| 11 | FERMT3 | fermitin family member 3 | 149781 | 168439 | 149743 | 41, 7, 21 |
| 11 | FDX1 | ferredoxin 1 | 137714 | 137710 | 137727 | 2, 43, 32 |
| 11 | FDXACB1 | ferredoxin-fold anticodon binding domain containing 1 | 255561 | 258529 | 137720 | 21, 37, 45 |
| 11 | FTH1 | ferritin heavy chain 1 | 167996 | 167995 | 149503 | |
| 11 | FIBP | FGF1 intracellular binding protein | 172500 | 172543 | 175602 | 18, 9, 34 |
| 11 | FGF19 | fibroblast growth factor 19 | 162344 | 281781 | 075388 | 29, 38, 16 |
| 11 | FGF3 | fibroblast growth factor 3 | 186895 | 075388 | 131620 | |
| 11 | FGF4 | fibroblast growth factor 4 | 075388 | 162344 | 186895 | 12, 38, |
| 11 | FLRT1 | fibronectin leucine rich transmembrane protein 1 | 126500 | 133315 | 168439 | 18, 14, 8 |
| 11 | FIBIN | fin bud initiation factor homolog (zebrafish) | 176971 | 148942 | 129151 | 11, 45, 15 |
| 11 | FKBP2 | FK506 binding protein 2 | 173486 | 173511 | 173457 | 31, 43, 29 |
| 11 | FEN1 | flap structure-specific endonuclease 1 | 168496 | 134825 | 134824 | 34, 21, 9 |
| 11 | FLI1 | Fli-1 proto-oncogene, ETS transcription factor | 151702 | 134954 | 151704 | 21, 45, 39 |
| 11 | FOLH1 | folate hydrolase 1 | 086205 | 214891 | 258817 | 26, 33, 36 |
| 11 | FOLR1 | folate receptor 1 | 110195 | 110203 | 165457 | 9, 27, 20 |
| 11 | FOLR3 | folate receptor 3 | 110203 | 110200 | 110195 | 17, 18, 6 |
| 11 | FOLR2 | folate receptor beta | 165457 | 110195 | 165458 | 1, 29, 30 |
| 11 | FSHB | follicle stimulating hormone beta subunit | 131808 | 182255 | 152219 | 31, 38, 8 |
| 11 | FOXR1 | forkhead box R1 | 176302 | 110375 | 186166 | 38, 8, |
| 11 | FNBP4 | formin binding protein 4 | 109920 | 165923 | 030066 | |
| 11 | FOSL1 | FOS like 1, AP-1 transcription factor subunit | 175592 | 175602 | 175573 | 19, 35, 37 |
| 11 | FJX1 | four jointed box 1 | 179431 | 149090 | 166326 | 27, 12, 6 |
| 11 | FZD4 | frizzled class receptor 4 | 174804 | 150687 | 166575 | 1, 11, 45 |
| 11 | FUT4 | fucosyltransferase 4 | 196371 | 257057 | 166025 | 3, 7, 42 |
| 11 | FXYD2 | FXYD domain containing ion transport regulator 2 | 137731 | 177103 | 255245 | 17, |
| 11 | FXYD6 | FXYD domain containing ion transport regulator 6 | 137726 | 255245 | 137747 | 26, 1, 8 |
| 11 | FXYD6-FXYD2 | FXYD6-FXYD2 readthrough | 255245 | 137731 | 137726 | |
| 11 | GNG3 | G protein subunit gamma 3 | 162188 | 234857 | 214753 | |
| 11 | GPR137 | G protein-coupled receptor 137 | 173264 | 002330 | 182450 | 38, 34, 28 |
| 11 | GPR152 | G protein-coupled receptor 152 | 175514 | 213402 | 175544 | |
| 11 | GPR83 | G protein-coupled receptor 83 | 123901 | 183560 | 020922 | 26, 40, 8 |
| 11 | GRK2 | G protein-coupled receptor kinase 2 | 173020 | 173120 | 172932 | 30, 21, 41 |
| 11 | GAL3ST3 | galactose-3-O-sulfotransferase 3 | 175229 | 175294 | 087365 | 14, 38, 8 |
| 11 | GLB1L2 | galactosidase beta 1 like 2 | 149328 | 166105 | 109956 | 29, 14, 38 |
| 11 | GLB1L3 | galactosidase beta 1 like 3 | 166105 | 151498 | 149328 | 38, 33, 35 |
| 11 | GAL | galanin and GMAP prepropeptide | 069482 | 110075 | 132749 | 35, |
| 11 | LGALS12 | galectin 12 | 133317 | 168004 | 133321 | 5, 2, 23 |
| 11 | BBOX1 | gamma-butyrobetaine hydroxylase 1 | 129151 | 176971 | 109881 | 17, 11, 13 |
| 11 | GIF | gastric intrinsic factor | 134812 | 166902 | 134827 | 37, 16, |
| 11 | GTF2H1 | general transcription factor IIH subunit 1 | 110768 | 110756 | 134333 | 45, 13, 26 |
| 11 | GANAB | glucosidase II alpha subunit | 089597 | 149541 | 185085 | 43, 18, 24 |
| 11 | GRIA4 | glutamate ionotropic receptor AMPA type subunit 4 | 152578 | 255501 | 170903 | 14, 8, 26 |
| 11 | GRIK4 | glutamate ionotropic receptor kainate type subunit 4 | 149403 | 196914 | 154114 | 38, 19, 8 |
| 11 | GRM5 | glutamate metabotropic receptor 5 | 168959 | 109861 | 077498 | |
| 11 | QSER1 | glutamine and serine rich 1 | 060749 | 135378 | 121690 | 11, 22, 42 |
| 11 | GSTP1 | glutathione S-transferase pi 1 | 084207 | 167791 | 167792 | 3, 30, 16 |
| 11 | GDPD4 | glycerophosphodiester phosphodiesterase domain containing 4 | 178795 | 137474 | 149269 | |
| 11 | GDPD5 | glycerophosphodiester phosphodiesterase domain containing 5 | 158555 | 149243 | 149257 | |
| 11 | GLYAT | glycine-N-acyltransferase | 149124 | 242689 | 156689 | 17, 19, |
| 11 | GLYATL1 | glycine-N-acyltransferase like 1 | 166840 | 156689 | 255151 | 12, |
| 11 | GLYATL1P3 | Glycine-N-acyltransferase like 1 pseudogene 3 | 255151 | 166840 | 189057 | |
| 11 | GLYATL2 | glycine-N-acyltransferase like 2 | 156689 | 149124 | 166840 | 7, 12, |
| 11 | PYGM | glycogen phosphorylase, muscle associated | 068976 | 068831 | 168066 | 15, 25, 24 |
| 11 | GPHA2 | glycoprotein hormone alpha 2 | 149735 | 068971 | 168070 | 38, 5, 20 |
| 11 | GRAMD1B | GRAM domain containing 1B | 023171 | 166250 | 166257 | 27, 32, 2 |
| 11 | GAB2 | GRB2 associated binding protein 2 | 033327 | 118369 | 137513 | 43, 35, 32 |
| 11 | GAS2 | growth arrest specific 2 | 148935 | 183161 | 198168 | 11, 34, 19 |
| 11 | GUCY1A2 | guanylate cyclase 1 soluble subunit alpha 2 | 152402 | 149313 | 152404 | 32, 1, 25 |
| 11 | H2AFX | H2A histone family member X | 188486 | 256269 | 172269 | 22, 27, 41 |
| 11 | HARBI1 | harbinger transposase derived 1 | 180423 | 110497 | 175224 | 31, 38, 37 |
| 11 | HSPA8 | heat shock protein family A (Hsp70) member 8 | 109971 | 188909 | 166250 | 45, 36, 17 |
| 11 | HSPB2 | heat shock protein family B (small) member 2 | 170276 | 109846 | 254445 | 25, 1, 15 |
| 11 | HBB | hemoglobin subunit beta | 244734 | 176742 | 223609 | 5, 7, 25 |
| 11 | HBD | hemoglobin subunit delta | 223609 | 244734 | 213934 | 5, 10, 7 |
| 11 | HBE1 | hemoglobin subunit epsilon 1 | 213931 | 279346 | 183251 | 36, 15, 11 |
| 11 | HBG1 | hemoglobin subunit gamma 1 | 213934 | 223609 | 196565 | 36, 15, 11 |
| 11 | HBG2 | hemoglobin subunit gamma 2 | 196565 | 213934 | 279824 | 36, 31, 11 |
| 11 | HPX | hemopexin | 110169 | 166313 | 110171 | 19, 23, 36 |
| 11 | HEPACAM | hepatic and glial cell adhesion molecule | 165478 | 154133 | 221932 | 8, 23, 19 |
| 11 | HEPN1 | hepatocellular carcinoma, down-regulated 1 | 221932 | 165478 | 149548 | 26, 8, 23 |
| 11 | HEPHL1 | hephaestin like 1 | 181333 | 214376 | 110218 | 30, 24, 25 |
| 11 | HNRNPUL2 | heterogeneous nuclear ribonucleoprotein U like 2 | 214753 | 162188 | 162222 | 41, 33, 16 |
| 11 | HIKESHI | Hikeshi, heat shock protein nuclear import factor | 149196 | 074266 | 149201 | 30, 34, 11 |
| 11 | HINFP | histone H4 transcription factor | 172273 | 172375 | 172350 | 39, 36, 14 |
| 11 | HTATIP2 | HIV-1 Tat interactive protein 2 | 109854 | 109851 | 185238 | 10, 37, 43 |
| 11 | HNRNPUL2- | HNRNPUL2-BSCL2 readthrough (NMD candidate) | 234857 | 177363 | 162188 | |
| | BSCL2 | | | | | |
| 11 | HIPK3 | homeodomain interacting protein kinase 3 | 110422 | 176102 | 110427 | 18, 29, 22 |
| 11 | HPS5 | HPS5, biogenesis of lysosomal organelles complex 2 subunit 2 | 110756 | 173432 | 110768 | 42, 22, 43 |
| 11 | HRASLS2 | HRAS like suppressor 2 | 133328 | 133321 | 176485 | 3, 37, 40 |
| 11 | HRASLS5 | HRAS like suppressor family member 5 | 168004 | 149742 | 133317 | 38, 17, 12 |
| 11 | HRAS | HRas proto-oncogene, GTPase | 174775 | 023191 | 161328 | 24, 6, 34 |
| 11 | HSPB2-C11orf52 | HSPB2-C11orf52 readthrough (NMD candidate) | 254445 | 170276 | 149300 | 30, 26, 14 |
| 11 | HMBS | hydroxymethylbilane synthase | 256269 | 160695 | 188486 | 21, 3, 7 |
| 11 | HSD17B12 | hydroxysteroid 17-beta dehydrogenase 12 | 149084 | 052841 | 166199 | 13, 17, 45 |
| 11 | HYLS1 | HYLS1, centriolar and ciliogenesis associated | 198331 | 237353 | 110060 | 6, 22, 38 |
| 11 | HYOU1 | hypoxia up-regulated 1 | 149428 | 137700 | 160695 | 23, 42, 45 |
| 11 | IGHMBP2 | immunoglobulin mu binding protein 2 | 132740 | 197345 | 172938 | 36, 38, 39 |
| 11 | IGSF22 | immunoglobulin superfamily member 22 | 179057 | 151117 | 110786 | 26, 29, 22 |
| 11 | IGSF9B | immunoglobulin superfamily member 9B | 080854 | 166118 | 166086 | 38, 32, |
| 11 | IPO7 | importin 7 | 205339 | 166471 | 166478 | 29, 24, 23 |
| 11 | INCENP | inner centromere protein | 149503 | 167996 | 168515 | 13, 39, 1 |
| 11 | IMMP1L | inner mitochondrial membrane peptidase subunit 1 | 148950 | 170946 | 109911 | 31, 33, 44 |
| 11 | INPPL1 | inositol polyphosphate phosphatase like 1 | 165458 | 165457 | 165462 | 26, 41, 20 |
| 11 | INSC | inscuteable homolog (Drosophila) | 188487 | 110680 | 110693 | 29, 34, 11 |
| 11 | INS-IGF2 | INS-IGF2 readthrough | 129965 | 167244 | 254647 | |
| 11 | INS | insulin | 254647 | 129965 | 180176 | |
| 11 | IGF2 | insulin like growth factor 2 | 167244 | 214026 | 129965 | 28, 26, 27 |
| 11 | INTS4 | integrator complex subunit 4 | 149262 | 087884 | 151364 | 9, 16, 43 |
| 11 | INTS5 | integrator complex subunit 5 | 185085 | 089597 | 255432 | 9, 32, 12 |
| 11 | ILK | integrin linked kinase | 166333 | 132275 | 166337 | 34, 40, 9 |
| 11 | IFITM1 | interferon induced transmembrane protein 1 | 185885 | 185201 | 142089 | 7, 34, 30 |
| 11 | IFITM10 | interferon induced transmembrane protein 10 | 244242 | 205864 | 250644 | 34, 6, 24 |
| 11 | IFITM2 | interferon induced transmembrane protein 2 | 185201 | 206013 | 185885 | 7, 34, 5 |
| 11 | IFITM3 | interferon induced transmembrane protein 3 | 142089 | 185885 | 182272 | 1, 43, 7 |
| 11 | IFITM5 | interferon induced transmembrane protein 5 | 206013 | 142102 | 185201 | 6, |
| 11 | IRF7 | interferon regulatory factor 7 | 185507 | 070047 | 099834 | 22, 5, 27 |
| 11 | IL10RA | interleukin 10 receptor subunit alpha | 110324 | 137747 | 255274 | 36, 39, 10 |
| 11 | IL18 | interleukin 18 | 150782 | 255292 | 150783 | 37, 13, 34 |
| 11 | IL18BP | interleukin 18 binding protein | 137496 | 137522 | 137497 | 37, 16, 33 |
| 11 | IFT46 | intraflagellar transport 46 | 118096 | 149582 | 095139 | 9, 31, 4 |
| 11 | IZUMO1R | IZUMO1 receptor, JUNO | 183560 | 110218 | 123901 | |
| 11 | JRKL | JRK-like | 183340 | 149231 | 149972 | |
| 11 | JAML | junction adhesion molecule like | 160593 | 149575 | 160588 | 36, 5, 39 |
| 11 | JAM3 | junctional adhesion molecule 3 | 166086 | 080854 | 151503 | 42, 1, 8 |
| 11 | KDELC2 | KDEL motif containing 2 | 178202 | 166323 | 110723 | 29, 1, 32 |
| 11 | KLHL35 | kelch like family member 35 | 149243 | 149273 | 158555 | 38, 15, 20 |
| 11 | KBTBD3 | kelch repeat and BTB domain containing 3 | 182359 | 170903 | 149313 | 31, 36, 2 |
| 11 | KBTBD4 | kelch repeat and BTB domain containing 4 | 123444 | 110536 | 196666 | 36, 38, 30 |
| 11 | KRTAP5-1 | keratin associated protein 5-1 | 205869 | 278919 | 205867 | 27, |
| 11 | KRTAP5-10 | keratin associated protein 5-10 | 204572 | 254997 | 204571 | 44, |
| 11 | KRTAP5-11 | keratin associated protein 5-11 | 204571 | 204572 | 158483 | 10, |
| 11 | KRTAP5-2 | keratin associated protein 5-2 | 205867 | 205869 | 196224 | 4, 10, 16 |
| 11 | KRTAP5-3 | keratin associated protein 5-3 | 196224 | 205867 | 241598 | |
| 11 | KRTAP5-4 | keratin associated protein 5-4 | 241598 | 196224 | 185940 | |
| 11 | KRTAP5-5 | keratin associated protein 5-5 | 185940 | 241598 | 205864 | 15, |
| 11 | KRTAP5-6 | keratin associated protein 5-6 | 205864 | 185940 | 244242 | |
| 11 | KRTAP5-7 | keratin associated protein 5-7 | 244411 | 172890 | 241233 | 38, |
| 11 | KRTAP5-8 | keratin associated protein 5-8 | 241233 | 244411 | 254997 | 38, |
| 11 | KRTAP5-9 | keratin associated protein 5-9 | 254997 | 241233 | 204572 | 24, 23, 38 |
| 11 | KIAA1549L | KIAA1549 like | 110427 | 110422 | 085063 | 8, 15, 14 |
| 11 | KIRREL3 | kin of IRRE like 3 (Drosophila) | 149571 | 110080 | 134954 | 15, 14, 36 |
| 11 | KIF18A | kinesin family member 18A | 121621 | 176697 | 169519 | 13, 10, 4 |
| 11 | KLC2 | kinesin light chain 2 | 174996 | 175115 | 174903 | 39, 26, 9 |
| 11 | LDHA | lactate dehydrogenase A | 134333 | 110768 | 166796 | 3, 13, 19 |
| 11 | LDHAL6A | lactate dehydrogenase A like 6A | 166800 | 166796 | 074319 | 38, 19, |
| 11 | LDHC | lactate dehydrogenase C | 166796 | 134333 | 166800 | 38, 10, 8 |
| 11 | LMNTD2 | lamin tail domain containing 2 | 185522 | 161328 | 099849 | |
| 11 | LARGE2 | LARGE xylosyl- and glucuronyltransferase 2 | 165905 | 121680 | 135365 | 18, 22, 32 |
| 11 | LAMTOR1 | late endosomal/lysosomal adaptor, MAPK and MTOR activator 1 | 149357 | 184154 | 110200 | 32, 27, 23 |
| 11 | LTBP3 | latent transforming growth factor beta binding protein 3 | 168056 | 142186 | 173465 | 14, 26, 10 |
| 11 | LAYN | layilin | 204381 | 183644 | 170145 | 6, 34, 8 |
| 11 | LBHD1 | LBH domain containing 1 | 162194 | 278615 | 214756 | 43, 30, 33 |
| 11 | LRP4 | LDL receptor related protein 4 | 134569 | 175216 | 149179 | 40, 16, 26 |
| 11 | LRP5 | LDL receptor related protein 5 | 162337 | 171067 | 110075 | 18, 16, 37 |
| 11 | LRFN4 | leucine rich repeat and fibronectin type III domain containing 4 | 173621 | 173599 | 173237 | 37, 3, 6 |
| 11 | LRRC10B | leucine rich repeat containing 10B | 204950 | 162148 | 011347 | 31, 29, 28 |
| 11 | LRRC32 | leucine rich repeat containing 32 | 137507 | 158636 | 182704 | 26, 28, 18 |
| 11 | LRRC4C | leucine rich repeat containing 4C | 148948 | 166352 | 166181 | 14, 15, 25 |
| 11 | LRRC55 | leucine rich repeat containing 55 | 183908 | 181273 | 134817 | 27, 8, 19 |
| 11 | LRRC56 | leucine rich repeat containing 56 | 161328 | 174775 | 185522 | 8, 38, 22 |
| 11 | LGR4 | leucine rich repeat containing G protein-coupled receptor 4 | 205213 | 109881 | 148943 | 11, 17, 45 |
| 11 | LRTOMT | leucine rich transmembrane and O-methyltransferase domain containing | 184154 | 137497 | 149357 | 40, 1, 9 |
| 11 | LUZP2 | leucine zipper protein 2 | 187398 | 255359 | 134343 | |
| 11 | LPXN | leupaxin | 110031 | 198283 | 186660 | 5, 36, 30 |
| 11 | LMO1 | LIM domain only 1 | 166407 | 166405 | 130413 | 28, 23, 14 |
| 11 | LMO2 | LIM domain only 2 | 135363 | 110429 | 135387 | 22, 45, 31 |
| 11 | LIN7C | lin-7 homolog C, crumbs cell polarity complex component | 148943 | 205213 | 176697 | 42, 18, 45 |
| 11 | LIPT2 | lipoyl(octanoyl) transferase 2 (putative) | 175536 | 175538 | 077514 | 4, 38, 12 |
| 11 | LDLRAD3 | low density lipoprotein receptor class A domain containing 3 | 179241 | 166326 | 110442 | 18, 42, 35 |
| 11 | LRRN4CL | LRRN4 C-terminal like | 177363 | 162191 | 234857 | 1, 9, 35 |
| 11 | LYVE1 | lymphatic vessel endothelial hyaluronan receptor 1 | 133800 | 110315 | 072952 | 36, 32, 1 |
| 11 | LSP1 | lymphocyte-specific protein 1 | 130592 | 130598 | 130595 | 41, 21, 2 |
| 11 | KAT5 | lysine acetyltransferase 5 | 172977 | 173039 | 172922 | 9, 1, 2 |
| 11 | KDM2A | lysine demethylase 2A | 173120 | 173156 | 173020 | 18, 22, 40 |
| 11 | KDM4D | lysine demethylase 4D | 186280 | 150316 | 235268 | 18, 38, 10 |
| 11 | KDM4E | lysine demethylase 4E | 235268 | 186280 | 263465 | |
| 11 | KMT2A | lysine methyltransferase 2A | 118058 | 167283 | 172425 | 22, 18, 26 |
| 11 | KMT5B | lysine methyltransferase 5B | 110066 | 110721 | 171067 | |
| 11 | MACROD1 | MACRO domain containing 1 | 133315 | 167770 | 126500 | 6, 14, 3 |
| 11 | MPEG1 | macrophage expressed 1 | 197629 | 110042 | 176495 | 36, 22, 42 |
| 11 | ME3 | malic enzyme 3 | 151376 | 149201 | 150687 | 29, 1, 2 |
| 11 | MADD | MAP kinase activating death domain | 110514 | 025434 | 134571 | 31, 43, 8 |
| 11 | MRGPRD | MAS related GPR family member D | 172938 | 132740 | 172935 | |
| 11 | MRGPRE | MAS related GPR family member E | 184350 | 182170 | 005801 | |
| 11 | MRGPRF | MAS related GPR family member F | 172935 | 172938 | 162341 | 44, 36, 43 |
| 11 | MRGPRG | MAS related GPR family member G | 182170 | 021762 | 184350 | |
| 11 | MRGPRX1 | MAS related GPR family member X1 | 170255 | 110786 | 183695 | |
| 11 | MRGPRX2 | MAS related GPR family member X2 | 183695 | 170255 | 177054 | 7, 38, 8 |
| 11 | MRGPRX3 | MAS related GPR family member X3 | 179826 | 166788 | 179817 | 14, 38, |
| 11 | MRGPRX4 | MAS related GPR family member X4 | 179817 | 179826 | 148965 | 25, |
| 11 | MAML2 | mastermind like transcriptional coactivator 2 | 184384 | 087053 | 149231 | 18, 42, 24 |
| 11 | MMP1 | matrix metallopeptidase 1 | 196611 | 166670 | 149968 | 45, 40, 10 |
| 11 | MMP10 | matrix metallopeptidase 10 | 166670 | 118113 | 196611 | 13, 18, 45 |
| 11 | MMP12 | matrix metallopeptidase 12 | 262406 | 149968 | 137745 | 23, 13, 18 |
| 11 | MMP13 | matrix metallopeptidase 13 | 137745 | 262406 | 137692 | 11, 6, 13 |
| 11 | MMP20 | matrix metallopeptidase 20 | 137674 | 137673 | 137675 | 10, 38, |
| 11 | MMP26 | matrix metallopeptidase 26 | 167346 | 167332 | 280021 | 10, 44, 8 |
| 11 | MMP27 | matrix metallopeptidase 27 | 137675 | 137674 | 118113 | 10, |
| 11 | MMP3 | matrix metallopeptidase 3 | 149968 | 196611 | 262406 | 10, 13, 18 |
| 11 | MMP7 | matrix metallopeptidase 7 | 137673 | 152558 | 137674 | 18, 23, 37 |
| 11 | MMP8 | matrix metallopeptidase 8 | 118113 | 137675 | 166670 | 5, 39, 7 |
| 11 | MED17 | mediator complex subunit 17 | 042429 | 182919 | 214376 | 11, 36, 45 |
| 11 | MED19 | mediator complex subunit 19 | 156603 | 156599 | 213593 | 29, 6, 41 |
| 11 | MCAM | melanoma cell adhesion molecule | 076706 | 110395 | 173456 | 45, 43, 24 |
| 11 | MTNR1B | melatonin receptor 1B | 134640 | 165323 | 180773 | |
| 11 | MAJIN | membrane anchored junction protein | 168070 | 149735 | 168062 | 31, 38, 36 |
| 11 | MFRP | membrane frizzled-related protein | 235718 | 223953 | 036672 | 14, 34, 28 |
| 11 | MS4A1 | membrane spanning 4-domains A1 | 156738 | 166930 | 071203 | 36, 22, 5 |
| 11 | MS4A10 | membrane spanning 4-domains A10 | 172689 | 166961 | 110104 | 16, |
| 11 | MS4A12 | membrane spanning 4-domains A12 | 071203 | 156738 | 204979 | 16, |
| 11 | MS4A13 | membrane spanning 4-domains A13 | 204979 | 071203 | 166959 | 38, |
| 11 | MS4A14 | membrane spanning 4-domains A14 | 166928 | 166927 | 166930 | 38, 22, 4 |
| 11 | MS4A15 | membrane spanning 4-domains A15 | 166961 | 214782 | 172689 | 20, 16, 10 |
| 11 | MS4A18 | membrane spanning 4-domains A18 | 214782 | 166959 | 166961 | |
| 11 | MS4A2 | membrane spanning 4-domains A2 | 149534 | 149516 | 110077 | 9, 5, 10 |
| 11 | MS4A3 | membrane spanning 4-domains A3 | 149516 | 149507 | 149534 | 34, 7, 5 |
| 11 | MS4A4A | membrane spanning 4-domains A4A | 110079 | 214787 | 166926 | 30, 32, 10 |
| 11 | MS4A4E | membrane spanning 4-domains A4E | 214787 | 110077 | 110079 | 15, |
| 11 | MS4A5 | membrane spanning 4-domains A5 | 166930 | 166928 | 156738 | 38, 30, |
| 11 | MS4A6A | membrane spanning 4-domains A6A | 110077 | 149534 | 214787 | |
| 11 | MS4A6E | membrane spanning 4-domains A6E | 166926 | 110079 | 166927 | 38, |
| 11 | MS4A7 | membrane spanning 4-domains A7 | 166927 | 166926 | 166928 | 22, 36, 29 |
| 11 | MS4A8 | membrane spanning 4-domains A8 | 166959 | 204979 | 214782 | 42, 30, 20 |
| 11 | MEN1 | menin 1 | 133895 | 168067 | 171219 | 21, 34, 22 |
| 11 | MPPED2 | metallophosphoesterase domain containing 2 | 066382 | 152219 | 170959 | 31, 25, 26 |
| 11 | MTA2 | metastasis associated 1 family member 2 | 149480 | 149016 | 149499 | 18, 3, 23 |
| 11 | METTL12 | methyltransferase like 12 | 214756 | 162194 | 204922 | 39, 36, 4 |
| 11 | METTL15 | methyltransferase like 15 | 169519 | 121621 | 182255 | 24, 42, 40 |
| 11 | MICALCL | MICAL C-terminal like | 133808 | 133816 | 197702 | |
| 11 | MARK2 | microtubule affinity regulating kinase 2 | 072518 | 168005 | 167771 | 30, 18, 21 |
| 11 | MICAL2 | microtubule associated monooxygenase, calponin and LIM domain containing 2 | 133816 | 050165 | 133808 | |
| 11 | MAP6 | microtubule associated protein 6 | 171533 | 149257 | 166391 | 31, 26, 8 |
| 11 | MDK | midkine (neurite growth-promoting factor 2) | 110492 | 149091 | 180720 | 12, 27, 37 |
| 11 | MTCH2 | mitochondrial carrier 2 | 109919 | 172247 | 165923 | 40, 38, 4 |
| 11 | MRPL11 | mitochondrial ribosomal protein L11 | 174547 | 174576 | 174516 | 29, 9, 21 |
| 11 | MRPL16 | mitochondrial ribosomal protein L16 | 166902 | 166900 | 134812 | 29, 9, 21 |
| 11 | MRPL17 | mitochondrial ribosomal protein L17 | 158042 | 166341 | 188124 | 1, 9, 21 |
| 11 | MRPL21 | mitochondrial ribosomal protein L21 | 197345 | 110090 | 132740 | 7, 3, 30 |
| 11 | MRPL23 | mitochondrial ribosomal protein L23 | 214026 | 130595 | 167244 | 31, 41, 22 |
| 11 | MRPL48 | mitochondrial ribosomal protein L48 | 175581 | 175582 | 181924 | 43, 15, 22 |
| 11 | MRPL49 | mitochondrial ribosomal protein L49 | 149792 | 162298 | 204710 | 2, 13, 28 |
| 11 | MAPK8IP1 | mitogen-activated protein kinase 8 interacting protein 1 | 121653 | 121671 | 234776 | 31, 26, 8 |
| 11 | MAP3K11 | mitogen-activated protein kinase kinase kinase 11 | 173327 | 173338 | 197136 | 34, 24, 36 |
| 11 | MAP4K2 | mitogen-activated protein kinase kinase kinase kinase 2 | 168067 | 168066 | 133895 | 7, 22, 41 |
| 11 | MOB2 | MOB kinase activator 2 | 182208 | 174672 | 184545 | 41, 15, 37 |
| 11 | MOGAT2 | monoacylglycerol O-acyltransferase 2 | 166391 | 171533 | 062282 | 37, 16, 42 |
| 11 | MRE11 | MRE11 homolog, double strand break repair nuclease | 020922 | 123901 | 168876 | |
| 11 | MTRNR2L8 | MT-RNR2-like 8 | 255823 | 133805 | 110315 | 40, 18, 2 |
| 11 | MUC15 | mucin 15, cell surface associated | 169550 | 134343 | 148942 | 32, 42, 11 |
| 11 | MUC5AC | mucin 5AC, oligomeric mucus/gel-forming | 215182 | 184956 | 117983 | |
| 11 | MUC5B | mucin 5B, oligomeric mucus/gel-forming | 117983 | 215182 | 078902 | 18, 16, 3 |
| 11 | MUC6 | mucin 6, oligomeric mucus/gel-forming | 184956 | 183020 | 215182 | 37, 33, 3 |
| 11 | MRVI1 | murine retrovirus integration site 1 homolog | 072952 | 133800 | 198730 | |
| 11 | MUS81 | MUS81 structure-specific endonuclease subunit | 172732 | 172803 | 172638 | 22, 45, 34 |
| 11 | MSANTD2 | Myb/SANT DNA binding domain containing 2 | 120458 | 149564 | 279342 | 22, 15, 26 |
| 11 | MSANTD4 | Myb/SANT DNA binding domain containing 4 with coiled-coils | 170903 | 152578 | 182359 | |
| 11 | MPZL2 | myelin protein zero like 2 | 149573 | 160588 | 198851 | 4, 30, 24 |
| 11 | MPZL3 | myelin protein zero like 3 | 160588 | 160593 | 149573 | 42, 29, 24 |
| 11 | MYRF | myelin regulatory factor | 124920 | 134780 | 134825 | 37, 8, 28 |
| 11 | MYEOV | myeloma overexpressed | 172927 | 162341 | 110092 | 3, 27, 16 |
| 11 | MYOD1 | myogenic differentiation 1 | 129152 | 188162 | 129159 | 25, 21, 15 |
| 11 | MYBPC3 | myosin binding protein C, cardiac | 134571 | 110514 | 066336 | 15, 19, 25 |
| 11 | MYO7A | myosin VIIA | 137474 | 254550 | 178795 | 1, 26, 32 |
| 11 | MTMR2 | myotubularin related protein 2 | 087053 | 166037 | 184384 | 44, 38, |
| 11 | NAA40 | N(alpha)-acetyltransferase 40, NatD catalytic subunit | 110583 | 167771 | 176340 | 39, 34, 40 |
| 11 | NAALAD2 | N-acetylated alpha-linked acidic dipeptidase 2 | 077616 | 255009 | 110172 | 31, 11, 38 |
| 11 | NAALADL1 | N-acetylated alpha-linked acidic dipeptidase like 1 | 168060 | 168061 | 146670 | 1, 26, 41 |
| 11 | NAT10 | N-acetyltransferase 10 | 135372 | 135387 | 166016 | 43, 24, 13 |
| 11 | NADSYN1 | NAD synthetase 1 | 172890 | 172893 | 244411 | |
| 11 | NDUFS3 | NADH:ubiquinone oxidoreductase core subunit S3 | 213619 | 149187 | 110536 | 29, 28, 31 |
| 11 | NDUFS8 | NADH:ubiquinone oxidoreductase core subunit S8 | 110717 | 006534 | 110719 | 1, 34, 14 |
| 11 | NDUFV1 | NADH:ubiquinone oxidoreductase core subunit V1 | 167792 | 084207 | 167799 | 3, 21, 15 |
| 11 | NDUFC2 | NADH:ubiquinone oxidoreductase subunit C2 | 151366 | 151365 | 159063 | 23, 31, 2 |
| 11 | NOX4 | NADPH oxidase 4 | 086991 | 077498 | 214414 | 17, 24, 45 |
| 11 | NCR3LG1 | natural killer cell cytotoxicity receptor 3 ligand 1 | 188211 | 070081 | 187486 | 29, 39, 23 |
| 11 | NDUFC2- | NDUFC2-KCTD14 readthrough | 259112 | 151364 | 151365 | |
| | KCTD14 | | | | | |
| 11 | NECTIN1 | nectin cell adhesion molecule 1 | 110400 | 154096 | 137699 | |
| 11 | NCAM1 | neural cell adhesion molecule 1 | 149294 | 188771 | 149292 | 8, 11, 26 |
| 11 | NELL1 | neural EGFL like 1 | 165973 | 165970 | 171714 | 8, 31, 25 |
| 11 | NEU3 | neuraminidase 3 | 162139 | 118363 | 171561 | 13, 7, 39 |
| 11 | NRXN2 | neurexin 2 | 110076 | 197891 | 068831 | 14, 26, 8 |
| 11 | NXPE1 | neurexophilin and PC-esterase domain family member 1 | 095110 | 076043 | 137634 | 16, 33, 38 |
| 11 | NXPE2 | neurexophilin and PC-esterase domain family member 2 | 204361 | 137634 | 182985 | 42, 25, |
| 11 | NXPE4 | neurexophilin and PC-esterase domain family member 4 | 137634 | 095110 | 204361 | 34, 16, 10 |
| 11 | NRGN | neurogranin | 154146 | 064199 | 019102 | 8, 6, 27 |
| 11 | NAV2 | neuron navigator 2 | 166833 | 129173 | 109851 | 40, 12, 20 |
| 11 | NPAS4 | neuronal PAS domain protein 4 | 174576 | 174669 | 174547 | 20, 44, 5 |
| 11 | NTM | neurotrimin | 182667 | 120451 | 183715 | 29, 11, 14 |
| 11 | NNMT | nicotinamide N-methyltransferase | 166741 | 109906 | 180425 | 19, 6, 34 |
| 11 | NLRX1 | NLR family member X1 | 160703 | 172350 | 172367 | 24, 42, 6 |
| 11 | NLRP10 | NLR family pyrin domain containing 10 | 182261 | 170683 | 175390 | |
| 11 | NLRP14 | NLR family pyrin domain containing 14 | 158077 | 149050 | 170748 | 45, 42, 8 |
| 11 | NLRP6 | NLR family pyrin domain containing 6 | 174885 | 185627 | 142102 | 42, 39, 5 |
| 11 | NCAPD3 | non-SMC condensin II complex subunit D3 | 151503 | 166086 | 151502 | 33, 29, 22 |
| 11 | NFRKB | nuclear factor related to kappaB binding protein | 170322 | 151715 | 170325 | 2, 6, 38 |
| 11 | NUMA1 | nuclear mitotic apparatus protein 1 | 137497 | 137496 | 184154 | 18, 33, 26 |
| 11 | NPAT | nuclear protein, coactivator of histone transcription | 149308 | 075239 | 149311 | 42, 22, 2 |
| 11 | NRIP3 | nuclear receptor interacting protein 3 | 175352 | 175348 | 175356 | 38, 8, 29 |
| 11 | NR1H3 | nuclear receptcr subfamily 1 group H member 3 | 025434 | 134575 | 110514 | 1, 36, 9 |
| 11 | NXF1 | nuclear RNA export factor 1 | 162231 | 185475 | 162236 | 39, 42, 44 |
| 11 | NUCB2 | nucleobindin 2 | 070081 | 011405 | 188211 | 31, 10, 33 |
| 11 | NUP160 | nucleoporin 160 | 030066 | 109920 | 149177 | 12, 6, 38 |
| 11 | NUP98 | nucleoporin 98 | 110713 | 129749 | 148985 | 38, 22, 6 |
| 11 | NAP1L4 | nucleosome assembly protein 1 like 4 | 205531 | 181649 | 110619 | 36, 39, 34 |
| 11 | NUDT22 | nudix hydrolase 22 | 149761 | 149743 | 110011 | 5, 4, 2 |
| 11 | NUDT8 | nudix hydrolase 8 | 167799 | 167792 | 167800 | 5, 33, 24 |
| 11 | OLFML1 | olfactomedin like 1 | 183801 | 170743 | 166387 | 36, 15, 42 |
| 11 | OMP | olfactory marker protein | 254550 | 149260 | 137474 | |
| 11 | OR1S1 | olfactory receptor family 1 subfamily S member 1 (gene/pseudogene) | 280204 | 197887 | 180475 | |
| 11 | OR1S2 | olfactory receptor family 1 subfamily S member 2 | 197887 | 186513 | 280204 | |
| 11 | OR10A2 | olfactpry receptor family 10 subfamily A member 2 | 170790 | 166363 | 170782 | |
| 11 | OR10A3 | olfactory receptor family 10 subfamily A member 3 | 170683 | 279000 | 182261 | |
| 11 | OR10A4 | olfactory receptor family 10 subfamily A member 4 | 170782 | 170790 | 166368 | |
| 11 | OR10A5 | olfactory receptor family 10 subfamily A member 5 | 166363 | 184933 | 170790 | |
| 11 | OR10A6 | olfactory receptor family 10 subfamily A member 6 (gene/pseudogene) | 279000 | 182334 | 170683 | 32, |
| 11 | OR10AG1 | olfactory receptor family 10 subfamily AG member 1 | 174970 | 167825 | 149133 | |
| 11 | OR10D3 | Olfactory receptor family 10 subfamily D member 3 (putative) | 197309 | 110002 | 197849 | |
| 11 | OR10G4 | olfactory receptor family 10 subfamily G member 4 | 254737 | 198674 | 236981 | |
| 11 | OR10G6 | Olfactory receptor family 10 subfamily G member 6 | 198674 | 196248 | 254737 | |
| 11 | OR10G7 | olfactory receptor family 10 subfamily G member 7 | 182634 | 234560 | 110002 | |
| 11 | OR10G8 | olfactory receptor family 10 subfamily G member 8 | 234560 | 236981 | 182634 | |
| 11 | OR10G9 | olfactory receptor family 10 subfamily G member 9 | 236981 | 254737 | 234560 | |
| 11 | OR10Q1 | olfactory receptor family 10 subfamily Q member 1 | 180475 | 280204 | 172772 | |
| 11 | OR10S1 | olfactory receptor family 10 subfamily S member 1 | 196248 | 181499 | 198674 | |
| 11 | OR10V1 | olfactory receptor family 10 subfamily V member 1 | 172289 | 166889 | 166900 | |
| 11 | OR10W1 | olfactory receptor family 10 subfamily W member 1 | 172772 | 180475 | 197786 | |
| 11 | OR2AG1 | olfactory receptor family 2 subfamily AG member 1 (gene/pseudogene) | 279486 | 188124 | 184933 | |
| 11 | OR2AG2 | olfactory receptor family 2 subfamily AG member 2 | 188124 | 158042 | 279486 | |
| 11 | OR2AT4 | olfactory receptor family 2 subfamily AT member 4 | 171561 | 162139 | 137491 | |
| 11 | OR2D2 | olfactory receptor family 2 subfamily D member 2 | 166368 | 170782 | 178358 | |
| 11 | OR2D3 | olfactory receptor family 2 subfamily D member 3 | 178358 | 166368 | 149054 | 19, 38, |
| 11 | OR4A15 | olfactory receptor family 4 subfamily A member 15 | 181958 | 181961 | 181939 | |
| 11 | OR4A16 | olfactory receptor family 4 subfamily A member 16 | 181961 | 150244 | 181958 | |
| 11 | OR4A47 | olfactory receptor family 4 subfamily A member 47 | 237388 | 176540 | 182053 | |
| 11 | OR4A5 | olfactory receptor family 4 subfamily A member 5 | 221840 | 225997 | 150244 | |
| 11 | OR4A8 | olfactory receptor family 4 subfamily A member 8 (gene/pseudogene) | 225997 | 185926 | 221840 | |
| 11 | OR4B1 | olfactory receptor family 4 subfamily B member 1 | 175619 | 149177 | 172208 | |
| 11 | OR4C11 | olfactory receptor family 4 subfamily C member 11 | 172188 | 279514 | 181927 | |
| 11 | OR4C12 | olfactory receptor family 4 subfamily C member 12 | 221954 | 258817 | 185926 | |
| 11 | OR4C13 | olfactory receptor family 4 subfamily C member 13 | 258817 | 086205 | 221954 | |
| 11 | OR4C15 | olfactory receptor family 4 subfamily C member 15 | 181939 | 181958 | 279514 | |
| 11 | OR4C16 | olfactory receptor family 4 subfamily C member 16 (gene/pseudogene) | 279514 | 181939 | 172188 | |
| 11 | OR4C3 | olfactory receptor family 4 subfamily C member 3 | 176547 | 176555 | 176540 | |
| 11 | OR4C46 | olfactory receptor family 4 subfamily C member 46 | 185926 | 221954 | 225997 | |
| 11 | OR4C5 | olfactory receptor family 4 subfamily C member 5 (gene/pseudogene) | 176540 | 176547 | 237388 | |
| 11 | OR4C6 | olfactory receptor family 4 subfamily C member 6 | 181903 | 174982 | 279761 | 32, |
| 11 | OR4D10 | olfactory receptor family 4 subfamily D member 10 | 254466 | 166884 | 176200 | |
| 11 | OR4D11 | olfactory receptor family 4 subfamily D member 11 | 176200 | 254466 | 172742 | |
| 11 | OR4D5 | olfactory receptor family 4 subfamily D member 5 | 171014 | 181518 | 181499 | |
| 11 | OR4D6 | olfactory receptor family 4 subfamily D member 6 | 166884 | 172320 | 254466 | |
| 11 | OR4D9 | olfactory receptor family 4 subfamily D member 9 | 172742 | 176200 | 110048 | |
| 11 | OR4P4 | olfactory receptor family 4 subfamily P member 4 | 181927 | 172188 | 174982 | |
| 11 | OR4S1 | olfactory receptor family 4 subfamily S member 1 | 176555 | 176567 | 176547 | |
| 11 | OR4S2 | olfactory receptor family 4 subfamily S member 2 | 174982 | 181927 | 181903 | |
| 11 | OR4X1 | olfactory receptor family 4 subfamily X member 1 (gene/pseudogene) | 176567 | 172208 | 176555 | |
| 11 | OR4X2 | olfactory receptor family 4 subfamily X member 2 (gene/pseudogene) | 172208 | 175619 | 176567 | |
| 11 | OR5A1 | olfactory receptor family 5 subfamily A member 1 | 172320 | 172324 | 166884 | |
| 11 | OR5A2 | olfactory receptor family 5 subfamily A member 2 | 172324 | 176495 | 172320 | |
| 11 | OR5AK2 | olfactory receptor family 5 subfamily AK member 2 | 181273 | 241356 | 183908 | |
| 11 | OR5AL1 | olfactory receptor family 5 subfamily AL member 1 (gene/pseudogene) | 272987 | 172199 | 279961 | |
| 11 | OR5AN1 | olfactory receptor family 5 subfamily AN member 1 | 176495 | 197629 | 172324 | |
| 11 | OR5AP2 | olfactory receptor family 5 subfamily AP member 2 | 172464 | 255012 | 172459 | 8, |
| 11 | OR5AR1 | olfactory receptor family 5 subfamily AR member 1 (gene/pseudogene) | 172459 | 172464 | 174914 | |
| 11 | OR5AS1 | olfactory receptor family 5 subfamily AS member 1 | 181785 | 149133 | 172154 | |
| 11 | OR5B12 | olfactory receptor family 5 subfamily B member 12 | 172362 | 172365 | 198283 | |
| 11 | OR5B17 | olfactory receptor family 5 subfamily B member 17 | 197786 | 172772 | 172769 | |
| 11 | OR5B2 | olfactory receptor family 5 subfamily B member 2 | 172365 | 172769 | 172362 | |
| 11 | OR5B21 | olfactory receptor family 5 subfamily B member 21 | 198283 | 172362 | 110031 | |
| 11 | OR5B3 | olfactory receptor family 5 subfamily B member 3 | 172769 | 197786 | 172365 | |
| 11 | OR5D13 | olfactory receptor family 5 subfamily D member 13 (gene/pseudogene) | 279761 | 181903 | 186113 | |
| 11 | OR5D14 | olfactory receptor family 5 subfamily D member 14 | 186113 | 279761 | 279395 | |
| 11 | OR5D16 | olfactory receptor family 5 subfamily D member 16 | 205029 | 205030 | 124900 | |
| 11 | OR5D18 | olfactory receptor family 5 subfamily D member 18 | 186119 | 279395 | 205030 | |
| 11 | OR5F1 | olfactory receptor family 5 subfamily F member 1 | 149133 | 174970 | 181785 | |
| 11 | OR5G3 | olfactory receptor family 5 subfamily G member 3 (gene/pseudogene) | 241356 | 172457 | 181273 | |
| 11 | OR5I1 | olfactory receptor family 5 subfamily I member 1 | 167825 | 187612 | 174970 | |
| 11 | OR5J2 | olfactory receptor family 5 subfamily J member 2 | 174957 | 181752 | 254658 | |
| 11 | OR5L1 | olfactory receptor family 5 subfamily L member 1 (gene/pseudogene) | 279395 | 186113 | 186119 | |
| 11 | OR5L2 | olfactory receptor family 5 subfamily L member 2 | 205030 | 186119 | 205029 | |
| 11 | OR5M1 | olfactory receptor family 5 subfamily M member 1 | 255012 | 254834 | 172464 | |
| 11 | OR5M10 | olfactory receptor family 5 subfamily M member 10 | 254834 | 255223 | 255012 | |
| 11 | OR5M11 | olfactory receptor family 5 subfamily M member 11 | 255223 | 181371 | 254834 | |
| 11 | OR5M3 | olfactory receptor family 5 subfamily M member 3 | 174937 | 150269 | 181371 | |
| 11 | OR5M8 | olfactory receptor family 5 subfamily M member 8 | 181371 | 174937 | 255223 | |
| 11 | OR5M9 | olfactory receptor family 5 subfamily M member 9 | 150269 | 279961 | 174937 | |
| 11 | OR5P2 | olfactory receptor family 5 subfamily P member 2 | 183303 | 183378 | 182334 | |
| 11 | OR5P3 | olfactory receptor family 5 subfamily P member 3 | 182334 | 183303 | 279000 | |
| 11 | OR5R1 | olfactory receptor family 5 subfamily R member 1 (gene/pseudogene) | 279961 | 272987 | 150269 | |
| 11 | OR5T1 | olfactory receptor family 5 subfamily T member 1 | 181698 | 172489 | 181693 | |
| 11 | OR5T2 | olfactory receptor family 5 subfamily T member 2 | 181718 | 254658 | 172489 | |
| 11 | OR5T3 | olfactory receptor family 5 subfamily T member 3 | 172489 | 181718 | 181698 | |
| 11 | OR5W2 | olfactory receptor family 5 subfamily W member 2 | 187612 | 124900 | 167825 | |
| 11 | OR51A2 | olfactory receptor family 51 subfamily A member 2 | 205496 | 205497 | 176798 | |
| 11 | OR51A4 | olfactory receptor family 51 subfamily A member 4 | 205497 | 278870 | 205496 | |
| 11 | OR51A7 | olfactory receptor family 51 subfamily A member 7 | 176895 | 176900 | 176893 | |
| 11 | OR51B2 | olfactory receptor family 51 subfamily B member 2 (gene/pseudogene) | 279012 | 183251 | 242180 | |
| 11 | OR51B4 | olfactory receptor family 51 subfamily B member 4 | 183251 | 213931 | 279012 | 34, |
| 11 | OR51B5 | olfactory receptor family 51 subfamily B member 5 | 242180 | 279012 | 176239 | 34, 7, 35 |
| 11 | OR51B6 | olfactory receptor family 51 subfamily B member 6 | 176239 | 242180 | 184698 | |
| 11 | OR51D1 | olfactory receptor family 51 subfamily D member 1 | 197428 | 167333 | 180785 | |
| 11 | OR51E1 | olfactory receptor family 51 subfamily E member 1 | 180785 | 197428 | 167332 | 17, 16, |
| 11 | OR51E2 | olfactory receptor family 51 subfamily E member 2 | 167332 | 180785 | 167346 | 33, 44, 20 |
| 11 | OR51F1 | olfactory receptor family 51 subfamily F member 1 (gene/pseudogene) | 280021 | 167346 | 279270 | |
| 11 | OR51F2 | olfactory receptor family 51 subfamily F member 2 | 176925 | 279270 | 176922 | |
| 11 | OR51G1 | olfactory receptor family 51 subfamily G member 1 (gene/pseudogene) | 278870 | 176893 | 205497 | |
| 11 | OR51G2 | olfactory receptor family 51 subfamily G member 2 | 176893 | 176895 | 278870 | |
| 11 | OR51H1 | olfactory receptor family 51 subfamily H member 1 | 176904 | 176922 | 176900 | |
| 11 | OR51I1 | olfactory receptor family 51 subfamily I member 1 | 167359 | 167360 | 187918 | |
| 11 | OR51I2 | olfactory receptor family 51 subfamily I member 2 | 187918 | 167359 | 181609 | |
| 11 | OR51J1 | olfactory receptor family 51 subfamily J member 1 (gene/pseudogene) | 184321 | 184698 | 167360 | |
| 11 | OR51L1 | olfactory receptor family 51 subfamily L member 1 | 176798 | 205496 | 205495 | |
| 11 | OR51M1 | olfactory receptor family 51 subfamily M member 1 | 184698 | 176239 | 184321 | |
| 11 | OR51Q1 | olfactory receptor family 51 subfamily Q member 1 (gene/pseudogene) | 167360 | 184321 | 167359 | |
| 11 | OR51S1 | olfactory receptor family 51 subfamily S member 1 | 176922 | 176925 | 176904 | |
| 11 | OR51T1 | olfactory receptor family 51 subfamily T member 1 | 176900 | 176904 | 176895 | |
| 11 | OR51V1 | olfactory receptor family 51 subfamily V member 1 | 176742 | 176748 | 244734 | |
| 11 | OR52A1 | olfactory receptor family 52 subfamily A member 1 | 182070 | 171944 | 176748 | |
| 11 | OR52A5 | olfactory receptor family 52 subfamily A member 5 | 171944 | 273085 | 182070 | |
| 11 | OR52B2 | olfactory receptor family 52 subfamily B member 2 | 255307 | 180919 | 175485 | |
| 11 | OR52B4 | olfactory receptor family 52 subfamily B member 4 (gene/pseudogene) | 221996 | 167325 | 132109 | 40, |
| 11 | OR52B6 | olfactory receptor family 52 subfamily B member 6 | 187747 | 181616 | 121236 | |
| 11 | OR52D1 | olfactory receptor family 52 subfamily D member 1 | 181609 | 187918 | 175520 | |
| 11 | OR52E1 | olfactory receptor family 52 subfamily E member 1 (gene/pseudogene) | 273085 | 176787 | 171944 | |
| 11 | OR52E2 | olfactory receptor family 52 subfamily E member 2 | 176787 | 205495 | 273085 | |
| 11 | OR52E4 | olfactory receptor family 52 subfamily E member 4 | 180974 | 183269 | 277932 | |
| 11 | OR52E5 | Olfactory receptor family 52 subfamily E member 5 | 277932 | 180974 | 184478 | |
| 11 | OR52E6 | olfactory receptor family 52 subfamily E member 6 | 205409 | 180988 | 183269 | |
| 11 | OR52E8 | olfactory receptor family 52 subfamily E member 8 | 183269 | 205409 | 180974 | |
| 11 | OR52H1 | olfactory receptor family 52 subfamily H member 1 | 181616 | 175518 | 187747 | |
| 11 | OR52I1 | olfactory receptor family 52 subfamily I member 1 | 232268 | 226288 | 167333 | |
| 11 | OR52I2 | olfactory receptor family 52 subfamily I member 2 | 226288 | 171987 | 232268 | |
| 11 | OR52J3 | olfactory receptor family 52 subfamily J member 3 | 205495 | 176798 | 176787 | |
| 11 | OR52K1 | olfactory receptor family 52 subfamily K member 1 | 196778 | 181963 | 197790 | |
| 11 | OR52K2 | olfactory receptor family 52 subfamily K member 2 | 181963 | 132109 | 196778 | |
| 11 | OR52L1 | olfactory receptor family 52 subfamily L member 1 | 183313 | 188691 | 183389 | |
| 11 | OR52M1 | olfactory receptor family 52 subfamily M member 1 | 197790 | 196778 | 171987 | |
| 11 | OR52N1 | olfactory receptor family 52 subfamily N member 1 | 181001 | 181009 | 180988 | |
| 11 | OR52N2 | olfactory receptor family 52 subfamily N member 2 | 180988 | 181001 | 205409 | |
| 11 | OR52N4 | olfactory receptor family 52 subfamily N member 4 (gene/pseudogene) | 181074 | 181023 | 181009 | |
| 11 | OR52N5 | olfactory receptor family 52 subfamily N member 5 | 181009 | 181074 | 181001 | |
| 11 | OR52R1 | olfactory receptor family 52 subfamily R member 1 (gene/pseudogene) | 279270 | 280021 | 176925 | |
| 11 | OR52W1 | olfactory receptor family 52 subfamily W member 1 | 175485 | 255307 | 180878 | |
| 11 | OR52Z1 | olfactory receptor family 52 subfamily Z member 1 (gene/pseudogene) | 176748 | 182070 | 176742 | |
| 11 | OR56A1 | olfactory receptor family 56 subfamily A member 1 | 180934 | 183389 | 180919 | |
| 11 | OR56A3 | olfactory receptor family 56 subfamily A member 3 | 184478 | 277932 | 188691 | |
| 11 | OR56A4 | olfactory receptor family 56 subfamily A member 4 | 183389 | 183313 | 180934 | |
| 11 | OR56A5 | olfactory receptor family 56 subfamily A member 5 | 188691 | 184478 | 183313 | |
| 11 | OR56B1 | olfactory receptor family 56 subfamily B member 1 | 181023 | 132274 | 181074 | 39, |
| 11 | OR56B4 | olfactory receptor family 56 subfamily B member 4 | 180919 | 180934 | 255307 | |
| 11 | OR6A2 | olfactory receptor family 6 subfamily A member 2 | 184933 | 279486 | 166363 | |
| 11 | OR6M1 | olfactory receptor family 6 subfamily M member 1 | 196099 | 221931 | 204300 | |
| 11 | OR6Q1 | olfactory receptor family 6 subfamily Q member 1 (gene/pseudogene) | 279051 | 186509 | 172377 | |
| 11 | OR6T1 | olfactory receptor family 6 subfamily T member 1 | 181499 | 171014 | 196248 | |
| 11 | OR6X1 | olfactory receptor family 6 subfamily X member 1 | 221931 | 166261 | 196099 | |
| 11 | OR8A1 | olfactory receptor family 8 subfamily A member 1 | 196119 | 170953 | 154143 | 32, |
| 11 | OR8B12 | olfactory receptor family 8 subfamily B member 12 | 170953 | 197125 | 196119 | |
| 11 | OR8B2 | olfactory receptor family 8 subfamily B member 2 | 204293 | 279116 | 196661 | |
| 11 | OR8B3 | olfactory receptor family 8 subfamily B member 3 | 196661 | 204293 | 280090 | 23, |
| 11 | OR8B4 | olfactory receptor family 8 subfamily B member 4 (gene/pseudogene) | 280090 | 196661 | 197125 | |
| 11 | OR8B8 | olfactory receptor family 8 subfamily B member 8 | 197125 | 280090 | 170953 | |
| 11 | OR8D1 | olfactory receptor family 8 subfamily D member 1 | 196341 | 255298 | 279116 | |
| 11 | OR8D2 | olfactory receptor family 8 subfamily D member 2 (gene/pseudogene) | 279116 | 196341 | 204293 | |
| 11 | OR8D4 | olfactory receptor family 8 subfamily D member 4 | 181518 | 204300 | 171014 | 25, 38, |
| 11 | OR8G1 | olfactory receptor family 8 subfamily G member 1 (gene/pseudogene) | 197849 | 197309 | 255298 | |
| 11 | OR8G5 | olfactory receptor family 8 subfamily G member 5 | 255298 | 197849 | 196341 | 38, |
| 11 | OR8H1 | olfactory receptor family 8 subfamily H member 1 | 181693 | 181698 | 280314 | |
| 11 | OR8H2 | olfactory receptor family 8 subfamily H member 2 | 181767 | 172154 | 181761 | |
| 11 | OR8H3 | olfactory receptor family 8 subfamily H member 3 | 181761 | 181767 | 167822 | |
| 11 | OR8I2 | olfactory receptor family 8 subfamily I member 2 | 172154 | 181785 | 181767 | |
| 11 | OR8J1 | olfactory receptor family 8 subfamily J member 1 | 172487 | 150261 | 172199 | |
| 11 | OR8J2 | olfactory receptor family 8 subfamily J member 2 (gene/pseudogene) | 254658 | 174957 | 181718 | |
| 11 | OR8J3 | olfactory receptor family 8 subfamily J member 3 | 167822 | 181761 | 181752 | |
| 11 | OR8K1 | olfactory receptor family 8 subfamily K member 1 | 150261 | 280314 | 172487 | |
| 11 | OR8K3 | olfactory receptor family 8 subfamily K member 3 (gene/pseudogene) | 280314 | 181693 | 150261 | |
| 11 | OR8K5 | olfactory receptor family 8 subfamily K member 5 | 181752 | 167822 | 174957 | |
| 11 | OR8U1 | olfactory receptor family 8 subfamily U member 1 | 172199 | 172487 | 272987 | |
| 11 | OR9G1 | olfactory receptor family 9 subfamily G member 1 | 174914 | 172459 | 172457 | |
| 11 | OR9G4 | olfactory receptor family 9 subfamily G member 4 | 172457 | 174914 | 241356 | |
| 11 | OR9I1 | olfactory receptor family 9 subfamily I member 1 | 172377 | 279051 | 186513 | |
| 11 | OR9Q1 | olfactory receptor family 9 subfamily Q member 1 | 186509 | 198561 | 279051 | |
| 11 | OR9Q2 | olfactory receptor family 9 subfamily Q member 2 | 186513 | 172377 | 197887 | |
| 11 | OOSP2 | oocyte secreted protein 2 | 149507 | 134827 | 149516 | 38, |
| 11 | OPCML | opioid binding protein/cell adhesion molecule like | 183715 | 182667 | 166118 | 29, 8, 14 |
| 11 | ORAOV1 | oral cancer overexpressed 1 | 149716 | 110092 | 281781 | 18, 40, 37 |
| 11 | OTOG | otogelin | 188162 | 006611 | 129152 | 38, |
| 11 | OTUB1 | OTU deubiquitinase, ubiquitin aldehyde binding 1 | 167770 | 256100 | 133315 | 3, 13, 43 |
| 11 | OAF | out at first homolog | 184232 | 137699 | 137709 | 19, 11, 9 |
| 11 | ODF3 | outer dense fiber of sperm tails 3 | 177947 | 188076 | 177963 | 38, |
| 11 | OVOL1 | ovo like transcriptional repressor 1 | 172818 | 254470 | 172757 | |
| 11 | OVCH2 | ovochymase 2 (gene/pseudogene) | 183378 | 166394 | 183303 | 24, 17, |
| 11 | OSBP | oxysterol binding protein | 110048 | 172742 | 166889 | 1, 14, 40 |
| 11 | OSBPL5 | oxysterol binding protein like 5 | 021762 | 110619 | 182170 | 39, 42, 26 |
| 11 | PAK1 | p21 (RAC1) activated kinase 1 | 149269 | 178795 | 074201 | 10, 24, 23 |
| 11 | PIDD1 | p53-induced death domain protein 1 | 177595 | 274897 | 177600 | 26, 25, 41 |
| 11 | PAX6 | paired box 6 | 007372 | 109911 | 049449 | 28, 14, 31 |
| 11 | PHOX2A | paired like homeobox 2a | 165462 | 165458 | 162129 | 26, 24, 8 |
| 11 | PANX1 | pannexin 1 | 110218 | 181333 | 183560 | 6, 1, 4 |
| 11 | PANX3 | pannexin 3 | 154143 | 196119 | 154144 | |
| 11 | PTH | parathyroid hormone | 152266 | 148925 | 197601 | 29, 10, 12 |
| 11 | PDDC1 | Parkinson disease 7 domain containing 1 | 177225 | 177156 | 184524 | 3, 34, 17 |
| 11 | PARVA | parvin alpha | 197702 | 133808 | 187079 | 42, 26, 29 |
| 11 | PATL1 | PAT1 homolog 1, processing body mRNA decay factor | 166889 | 110048 | 172289 | 23, 5, 9 |
| 11 | PNPLA2 | patatin like phospholipase domain containing 2 | 177666 | 177600 | 177685 | 22, 6, 28 |
| 11 | PKNOX2 | PBX/knotted 1 homeobox 2 | 165495 | 150433 | 149557 | 40, 8, 14 |
| 11 | PCF11 | PCF11 cleavage and polyadenylation factor subunit | 165494 | 137502 | 137494 | |
| 11 | PDZD3 | PDZ domain containing 3 | 172367 | 160703 | 248712 | 17, 16, 3 |
| 11 | PCNX3 | pecanex homolog 3 (Drosophila) | 197136 | 173327 | 213445 | 26, 31, 5 |
| 11 | PELI3 | pellino E3 ubiquitin protein ligase family member 3 | 174516 | 174547 | 254986 | 1, 27, 26 |
| 11 | PGA3 | pepsinogen 3, group I (pepsinogen A) | 229859 | 167987 | 229183 | 37, 13, 31 |
| 11 | PGA4 | pepsinogen 4, group I (pepsinogen A) | 229183 | 229859 | 256713 | 37, 13, 25 |
| 11 | PGA5 | pepsinogen 5, group I (pepsinogen A) | 256713 | 229183 | 167992 | 37, 13, 17 |
| 11 | PAMR1 | peptidase domain containing associated with muscle regeneration 1 | 149090 | 110436 | 179431 | 45, 1, 35 |
| 11 | PRDX5 | peroxiredoxin 5 | 126432 | 173113 | 168071 | 41, 43, 2 |
| 11 | PEX16 | peroxisomal biogenesis factor 16 | 121680 | 234776 | 165905 | 6, 3, 26 |
| 11 | PHRF1 | PHD and ring finger domains 1 | 070047 | 099849 | 185507 | 21, 34, 41 |
| 11 | PHF21A | PHD finger protein 21A | 135365 | 165905 | 157613 | 9, 22, 7 |
| 11 | PICALM | phosphatidylinositol binding clathrin assembly protein | 073921 | 150676 | 074266 | 29, 26, 40 |
| 11 | PITPNM1 | phosphatidylinositol transfer protein membrane associated 1 | 110697 | 110711 | 167797 | 34, 18, 1 |
| 11 | PIK3C2A | phosphatidylinositol-4-phosphate 3-kinase catalytic subunit type 2 alpha | 011405 | 110700 | 070081 | 45, 40, 25 |
| 11 | PTDSS2 | phosphatidylserine synthase 2 | 174915 | 185101 | 023191 | 34, 38, 21 |
| 11 | PDE2A | phosphodiesterase 2A | 186642 | 162129 | 186635 | 9, 8, 36 |
| 11 | PDE3B | phosphodiesterase 3B | 152270 | 129084 | 186104 | 28, 31, 45 |
| 11 | PACS1 | phosphofurin acidic cluster sorting protein 1 | 175115 | 087365 | 174996 | 34, 41, 39 |
| 11 | PGM2L1 | phosphoglucomutase 2 like 1 | 165434 | 149380 | 175538 | 6, 8, 43 |
| 11 | PLA2G16 | phospholipase A2 group XVI | 176485 | 133328 | 184743 | 1, 26, 11 |
| 11 | PLCB3 | phospholipase C beta 3 | 149782 | 173457 | 002330 | 1, 35, 16 |
| 11 | PIH1D2 | PIH1 domain containing 2 | 150773 | 150768 | 150776 | 43, 31, 30 |
| 11 | PIWIL4 | piwi like RNA-mediated gene silencing 4 | 134627 | 257057 | 166025 | 10, 26, 38 |
| 11 | PLET1 | placenta expressed transcript 1 | 188771 | 150787 | 149294 | |
| 11 | PKP3 | plakophilin 3 | 184363 | 182272 | 185187 | 24, 16, 28 |
| 11 | PAFAH1B2 | platelet activating factor acetylhydrolase 1b catalytic subunit 2 | 168092 | 160584 | 149577 | 24, 37, 26 |
| 11 | PDGFD | platelet derived growth factor D | 170962 | 187240 | 170967 | 42, 2, 22 |
| 11 | PLEKHA7 | pleckstrin homology domain containing A7 | 166689 | 110696 | 110700 | 26, 40, 17 |
| 11 | PLEKHB1 | pleckstrin homology domain containing B1 | 021300 | 054965 | 175582 | 8, 14, 17 |
| 11 | PHLDA2 | pleckstrin homology like domain family A member 2 | 181649 | 110628 | 205531 | 32, 28, 16 |
| 11 | PHLDB1 | pleckstrin homology like domain family B member 1 | 019144 | 095139 | 118094 | 26, 27, 12 |
| 11 | GALNT18 | polypeptide N-acetylgalactosaminyltransferase 18 | 110328 | 236287 | 254598 | 24, 36, 8 |
| 11 | PGAP2 | post-GPI attachment to proteins 2 | 148985 | 110713 | 177105 | 21, 38, 5 |
| 11 | KCTD14 | potassium channel tetramerization domain containing 14 | 151364 | 149262 | 259112 | 18, 6, 27 |
| 11 | KCTD21 | potassium channel tetramerization domain containing 21 | 188997 | 159063 | 118369 | 43, 24, 8 |
| 11 | KCNK4 | potassium two pore domain channel subfamily K member 4 | 182450 | 173264 | 219435 | 25, 8, 27 |
| 11 | KCNK7 | potassium two pore domain channel subfamily K member 7 | 173338 | 173442 | 173327 | 15, 32, 20 |
| 11 | KCNA4 | potassium voltage-gated channel subfamily A member 4 | 182255 | 169519 | 131808 | 22, 20, 8 |
| 11 | KCNC1 | potassium voltage-gated channel subfamily C member 1 | 129159 | 129152 | 129158 | 14, 8, 30 |
| 11 | KCNE3 | potassium voltage-gated channel subfamily E regulatory subunit 3 | 175538 | 165434 | 175536 | 1, 37, 3 |
| 11 | KCNJ1 | potassium voltage-gated channel subfamily J member 1 | 151704 | 151702 | 120457 | 17, 40, 42 |
| 11 | KCNJ11 | potassium voltage-gated channel subfamily J member 11 | 187486 | 188211 | 006071 | |
| 11 | KCNJ5 | potassium voltage-gated channel subfamily J member 5 | 120457 | 151704 | 174370 | 2, 39, 36 |
| 11 | KCNQ1 | potassium voltage-gated channel subfamily Q member 1 | 053918 | 070985 | 129757 | 40, 2, 16 |
| 11 | POU2AF1 | POU class 2 associating factor 1 | 110777 | 214290 | 137707 | |
| 11 | POU2F3 | POU class 2 homeobox 3 | 137709 | 184232 | 181264 | 13, 16, 3 |
| 11 | PPFIBP2 | PPFIA binding protein 2 | 166387 | 183801 | 166394 | |
| 11 | PRDM10 | PR/SET domain 10 | 170325 | 170322 | 084234 | 13, 12, 7 |
| 11 | PRDM11 | PR/SET domain 11 | 019485 | 175274 | 019505 | 43, 9, 26 |
| 11 | PRPF19 | pre-mRNA processing factor 19 | 110107 | 149506 | 110108 | 9, 21, 3 |
| 11 | PANO1 | proapoptotic nucleolar protein 1 | 274897 | 177542 | 177595 | |
| 11 | PGR | progesterone receptor | 082175 | 170647 | 137672 | 25, 44, 26 |
| 11 | PRR5L | proline rich 5 like | 135362 | 110442 | 175104 | 36, 24, 42 |
| 11 | PRRG4 | proline rich and Gla domain 4 | 135378 | 186714 | 060749 | 32, 15, 44 |
| 11 | P4HA3 | prolyl 4-hydroxylase subunit alpha 3 | 149380 | 214517 | 165434 | 11, 24, 18 |
| 11 | PRCP | prolylcarboxypeptidase | 137509 | 182103 | 165490 | 43, 13, 15 |
| 11 | PCSK7 | proprotein convertase subtilisin/kexin type 7 | 160613 | 149591 | 167257 | 43, 39, 34 |
| 11 | PTGDR2 | prostaglandin D2 receptor 2 | 183134 | 110104 | 149506 | 28, 39, 15 |
| 11 | PATE1 | prostate and testis expressed 1 | 171053 | 134940 | 196844 | 33, 38, 8 |
| 11 | PATE2 | prostate and testis expressed 2 | 196844 | 171053 | 236027 | 33, |
| 11 | PATE3 | prostate and testis expressed 3 | 236027 | 196844 | 237353 | 38, |
| 11 | PATE4 | prostate and testis expressed 4 | 237353 | 236027 | 198331 | 33, |
| 11 | PRSS23 | protease, serine 23 | 150687 | 151376 | 174804 | |
| 11 | PAAF1 | proteasomal ATPase associated factor 1 | 175575 | 181924 | 187726 | 1, 30, 9 |
| 11 | PSMC3 | proteasome 26S subunit, ATPase 3 | 165916 | 165915 | 165917 | 34, 21, 9 |
| 11 | PSMD13 | proteasome 26S subunit, non-ATPase 13 | 185627 | 142082 | 174885 | 3, 30, 21 |
| 11 | PSMA1 | proteasome subunit alpha 1 | 129084 | 256206 | 152270 | 30, 34, 7 |
| 11 | PRMT3 | protein arginine methyltransferase 3 | 185238 | 109854 | 165970 | 6, 10, 31 |
| 11 | PACSIN3 | protein kinase C and casein kinase substrate in neurons 3 | 165912 | 149182 | 134574 | 9, 25, 1 |
| 11 | PRKCDBP | protein kinase C delta binding protein | 170955 | 110148 | 166311 | 34, 35, 38 |
| 11 | PPP1CA | protein phosphatase 1 catalytic subunit alpha | 172531 | 175505 | 175463 | 41, 21, 9 |
| 11 | PPP1R14B | protein phosphatase 1 regulatory inhibitor subunit 14B | 173457 | 173486 | 149782 | 15, 34, 22 |
| 11 | PPP1R32 | protein phosphatase 1 regulatory subunit 32 | 162148 | 167985 | 204950 | 38, 27, 14 |
| 11 | PPP2R5B | protein phosphatase 2 regulatory subunit B′beta | 068971 | 110046 | 149735 | 34, 26, 40 |
| 11 | PPP2R1B | protein phosphatase 2 scaffold subunit Abeta | 137713 | 170145 | 086848 | 1, 38, 45 |
| 11 | PPP6R3 | protein phosphatase 6 regulatory subunit 3 | 110075 | 162337 | 069482 | 42, 22, 7 |
| 11 | PPME1 | protein phosphatase methylesterase 1 | 214517 | 168014 | 149380 | 17, 37, 13 |
| 11 | PTPMT1 | protein tyrosine phosphatase, mitochondrial 1 | 110536 | 213619 | 123444 | |
| 11 | PTPN5 | protein tyrosine phosphatase, non-receptor type 5 | 110786 | 179057 | 170255 | 8, 38, 17 |
| 11 | PTPRCAP | protein tyrosine phosphatase, receptor type C associated protein | 213402 | 175634 | 175514 | 41, 21, 22 |
| 11 | PTPRJ | protein tyrosine phosphatase, receptor type J | 149177 | 030066 | 175619 | 17, 37, 6 |
| 11 | PGGHG | protein-glucosylgalactosylhydroxylysine glucosidase | 142102 | 174885 | 206013 | 39, 20, 1 |
| 11 | PRG2 | proteoglycan 2, pro eosinophil major basic protein | 186652 | 156575 | 254979 | 32, 7, 5 |
| 11 | PRG3 | proteoglycan 3, pro eosinophil major basic protein 2 | 156575 | 109991 | 186652 | 7, |
| 11 | PUS3 | pseudouridylate synthase 3 | 110060 | 198331 | 109832 | 31, 25, 26 |
| 11 | PPFIA1 | PTPRF interacting protein alpha 1 | 131626 | 168040 | 085733 | 18, 26, 9 |
| 11 | P2RX3 | purinergic receptor P2X 3 | 109991 | 149136 | 156575 | 38, 15, 12 |
| 11 | P2RY2 | purinergic receptor P2Y2 | 175591 | 137478 | 171631 | 37, 2, 5 |
| 11 | P2RY6 | pyrimidinergic receptor P2Y6 | 171631 | 175591 | 110237 | 36, 32, 26 |
| 11 | PC | pyruvate carboxylase | 173599 | 173653 | 173621 | 1, 9, 6 |
| 11 | PDHX | pyruvate dehydrogenase complex component X | 110435 | 149089 | 026508 | 3, 34, 15 |
| 11 | RAB1B | RAB1B, member RAS oncogene family | 174903 | 174996 | 174871 | 26, 21, 33 |
| 11 | RAB30 | RAB30, member RAS oncogene family | 137502 | 165490 | 165494 | 22, 13, 18 |
| 11 | RAB38 | RAB38, member RAS oncogene family | 123892 | 166575 | 109861 | 35, 31, 23 |
| 11 | RAB39A | RAB39A, member RAS oncogene family | 179331 | 110660 | 166266 | 42, 14, 12 |
| 11 | RAB3IL1 | RAB3A interacting protein like 1 | 167994 | 221968 | 167995 | 1, 34, 27 |
| 11 | RAB6A | RAB6A, member RAS oncogene family | 175582 | 021300 | 175581 | 8, 4, 34 |
| 11 | RAD9A | RAD9 checkpoint clamp component A | 172613 | 172830 | 175482 | 21, 27, 25 |
| 11 | RDX | radixin | 137710 | 149289 | 137714 | 11, 4, 6 |
| 11 | RIN1 | Ras and Rab interactor 1 | 174791 | 174807 | 174744 | 34, 7, 27 |
| 11 | RASSF10 | Ras association domain family member 10 | 189431 | 187079 | 133794 | 24, 2, 23 |
| 11 | RASSF7 | Ras association domain family member 7 | 099849 | 185522 | 070047 | 37, 35, 3 |
| 11 | RCE1 | Ras converting CAAX endopeptidase 1 | 173653 | 173715 | 173599 | 27, 18, 22 |
| 11 | RASGRP2 | RAS guanyl releasing protein 2 | 068831 | 110076 | 068976 | 36, 5, 7 |
| 11 | RHOD | ras homolog family member D | 173156 | 173227 | 173120 | 9, 22, 33 |
| 11 | RHOG | ras homolog family member G | 177105 | 148985 | 167323 | 5, 41, 31 |
| 11 | RBM14-RBM4 | RBM14-RBM4 readthrough | 248643 | 239306 | 173933 | |
| 11 | RAPSN | receptor associated protein of the synapse | 165917 | 165916 | 149187 | 25, 15, 14 |
| 11 | RAG1 | recombination activating 1 | 166349 | 175104 | 175097 | |
| 11 | RAG2 | recombination activating 2 | 175097 | 166349 | 166352 | 39, 22, 15 |
| 11 | RELA | RELA proto-oncogene, NF-kB subunit | 173039 | 213445 | 172977 | 36, 39, 24 |
| 11 | RRAS2 | related RAS viral (r-ras) oncogene homolog 2 | 133818 | 262655 | 129083 | 22, 6, 26 |
| 11 | RELT | RELT tumor necrosis factor receptor | 054967 | 110237 | 054965 | 36, 5, 39 |
| 11 | RSF1 | remodeling and spacing factor 1 | 048649 | 178301 | 087884 | 27, 9, 28 |
| 11 | RCOR2 | REST corepressor 2 | 167771 | 072518 | 110583 | 14, 26, 12 |
| 11 | RCN1 | reticulocalbin 1 | 049449 | 007372 | 184937 | |
| 11 | RTN3 | reticulon 3 | 133318 | 184743 | 188070 | 8, 13, 38 |
| 11 | RTN4RL2 | reticulon 4 receptor-like 2 | 186907 | 134802 | 149150 | 27, 2, 40 |
| 11 | ROM1 | retinal outer segment membrane protein 1 | 149489 | 149499 | 149541 | 14, 24, 26 |
| 11 | RARRES3 | retinoic acid receptor responder 3 | 133321 | 133317 | 133328 | 37, 3, 4 |
| 11 | ARHGAP1 | Rho GTPase activating protein 1 | 175220 | 175224 | 175213 | 28, 30, 15 |
| 11 | ARHGAP20 | Rho GTPase activating protein 20 | 137727 | 137714 | 150750 | 1, 25, 38 |
| 11 | ARHGAP32 | Rho GTPase activating protein 32 | 134909 | 120471 | 043039 | 26, 30, 11 |
| 11 | ARHGAP42 | Rho GTPase activating protein 42 | 165895 | 149972 | 170647 | 26, 29, 40 |
| 11 | ARHGEF12 | Rho guanine nucleotide exchange factor 12 | 196914 | 181264 | 149403 | 45, 11, 1 |
| 11 | ARHGEF17 | Rho guanine nucleotide exchange factor 17 | 110237 | 171631 | 054967 | 43, 26, 40 |
| 11 | RNASEH2C | ribonuclease H2 subunit C | 172922 | 172977 | 254470 | 36, 2, 39 |
| 11 | RNH1 | ribonuclease/angiogenin inhibitor 1 | 023191 | 174915 | 174775 | 3, 35, 9 |
| 11 | RRM1 | ribonucleotide reductase catalytic subunit M1 | 167325 | 167323 | 221996 | 13, 39, 18 |
| 11 | RPL27A | ribosomal protein L27a | 166441 | 166436 | 166444 | |
| 11 | RPLP2 | ribosomal protein lateral stalk subunit P2 | 177600 | 177595 | 177666 | 11, 43, 1 |
| 11 | RPS13 | ribosomal protein S13 | 110700 | 166689 | 011405 | 43, 33, 7 |
| 11 | RPS25 | ribosomal protein S25 | 118181 | 186166 | 196655 | 43, 41, 25 |
| 11 | RPS3 | ribosomal protein S3 | 149273 | 137486 | 149243 | 41, 21, 27 |
| 11 | RPS6KA4 | ribosomal protein S6 kinase A4 | 162302 | 168071 | 168065 | 9, 13, 28 |
| 11 | RPS6KB2 | ribosomal protein S6 kinase B2 | 175634 | 172508 | 213402 | 37, 21, 34 |
| 11 | RRP8 | ribosomal RNA processing 8, methyltransferase, homolog (yeast) | 132275 | 179532 | 166333 | 41, 35, 18 |
| 11 | RIC3 | RIC3 acetylcholine receptor chaperone | 166405 | 166402 | 166407 | 2, 1, 26 |
| 11 | RIC8A | RIC8 guanine nucleotide exchange factor A | 177963 | 177947 | 142082 | 35, 38, 36 |
| 11 | RNF121 | ring finger protein 121 | 137522 | 225805 | 137496 | 30, 43, 4 |
| 11 | RNF141 | ring finger protein 141 | 110315 | 255823 | 133800 | 38, 8, 41 |
| 11 | RNF169 | ring finger protein 169 | 166439 | 054938 | 166435 | |
| 11 | RNF214 | ring finger protein 214 | 167257 | 160613 | 186318 | 9, 23, 39 |
| 11 | RNF26 | ring finger protein 26 | 173456 | 076706 | 223953 | 24, 39, 35 |
| 11 | RBM14 | RNA binding motif protein 14 | 239306 | 173992 | 248643 | 24, 40, 16 |
| 11 | RBM4 | RNA binding motif protein 4 | 173933 | 248643 | 173914 | 30, 9, 22 |
| 11 | RBM4B | RNA binding motif protein 4B | 173914 | 173933 | 173898 | |
| 11 | RBM7 | RNA binding motif protein 7 | 076053 | 282682 | 255663 | 26, 7, 29 |
| 11 | RBMXL2 | RNA binding motif protein, X-linked like 2 | 170748 | 158077 | 170743 | 38, 17, 8 |
| 11 | REXO2 | RNA exonuclease 2 | 076043 | 255663 | 095110 | 33, 19, 12 |
| 11 | POLR2G | RNA polymerase II subunit G | 168002 | 185670 | 162227 | 7, 30, 34 |
| 11 | POLR2L | RNA polymerase II subunit L | 177700 | 177697 | 214063 | 25, 1, 34 |
| 11 | RPUSD4 | RNA pseudouridylate synthase domain containing 4 | 165526 | 064309 | 197798 | 3, 18, 39 |
| 11 | ROBO3 | roundabout guidance receptor 3 | 154134 | 279342 | 154133 | 2, 36, 26 |
| 11 | ROBO4 | roundabout guidance receptor 4 | 154133 | 154134 | 165478 | 43, 36, 45 |
| 11 | SAA2-SAA4 | SAA2-SAA4 readthrough | 255071 | 148965 | 134339 | |
| 11 | SAC3D1 | SAC3 domain containing 1 | 168061 | 110025 | 168060 | 31, 1, 26 |
| 11 | SIK2 | salt inducible kinase 2 | 170145 | 204381 | 137713 | 1, 26, 23 |
| 11 | SLN | sarcolipin | 170290 | 110675 | 110660 | 25, 10, 15 |
| 11 | SCYL1 | SCY1 like pseudokinase 1 | 142186 | 279576 | 168056 | 7, 1, 22 |
| 11 | SCT | secretin | 070031 | 099834 | 069696 | 38, |
| 11 | SERGEF | secretion regulating guanine nucleotide exchange factor | 129158 | 129159 | 129167 | 41, 10, 1 |
| 11 | SCGB1A1 | secretoglobin family 1A member 1 | 149021 | 162174 | 124942 | 20, 30, 33 |
| 11 | SCGB1C1 | secretoglobin family 1C member 1 | 188076 | 177951 | 177947 | 17, 10, 38 |
| 11 | SCGB1D1 | secretoglobin family 1D member 1 | 168515 | 149503 | 124939 | 14, |
| 11 | SCGB1D2 | secretoglobin family 1D member 2 | 124935 | 124939 | 110484 | 27, 25, 44 |
| 11 | SCGB1D4 | secretoglobin family 1D member 4 | 197745 | 110484 | 162174 | 44, |
| 11 | SCGB2A1 | secretoglobin family 2A member 1 | 124939 | 168515 | 124935 | 14, 40, 44 |
| 11 | SCGB2A2 | secretoglobin family 2A member 2 | 110484 | 124935 | 197745 | 23, 10, 27 |
| 11 | SELENOH | selenoprotein H | 211450 | 254462 | 254732 | |
| 11 | SRSF8 | serine and arginine rich splicing factor 8 | 263465 | 235268 | 149218 | 29, 24, 40 |
| 11 | STK33 | serine/threonine kinase 33 | 130413 | 166407 | 166436 | 38, 10, 20 |
| 11 | SERPING1 | serpin family G member 1 | 149131 | 156587 | 166793 | 1, 4, 43 |
| 11 | SERPINH1 | serpin family H member 1 | 149257 | 158555 | 171533 | 45, 10, 13 |
| 11 | SAAL1 | serum amyloid A like 1 | 166788 | 129167 | 179826 | 41, 35, 19 |
| 11 | SAA1 | serum amyloid A1 | 173432 | 134339 | 110756 | 19, 1, 30 |
| 11 | SAA2 | serum amyloid A2 | 134339 | 255071 | 173432 | 19, 25, 1 |
| 11 | SAA4 | serum amyloid A4, constitutive | 148965 | 179817 | 255071 | 19, 30, 17 |
| 11 | SESN3 | sestrin 3 | 149212 | 149218 | 077458 | |
| 11 | SBF2 | SET binding factor 2 | 133812 | 133789 | 148926 | 26, 11, 45 |
| 11 | SHANK2 | SH3 and multiple ankyrin repeat domains 2 | 162105 | 085733 | 172893 | 11, 42, 18 |
| 11 | SIAE | sialic acid acetylesterase | 110013 | 154144 | 064199 | |
| 11 | SIDT2 | SID1 transmembrane family member 2 | 149577 | 168092 | 149591 | 26, 40, 38 |
| 11 | SPCS2 | signal peptidase complex subunit 2 | 118363 | 166435 | 162139 | |
| 11 | SCUBE2 | signal peptide, CUB domain and EGF like domain containing 2 | 175356 | 175352 | 184014 | 23, 33, 15 |
| 11 | SIPA1 | signal-induced proliferation-associated 1 | 213445 | 197136 | 173039 | 41, 22, 3 |
| 11 | SIK3 | SIK family kinase 3 | 160584 | 118137 | 168092 | 18, 26, 20 |
| 11 | SIGIRR | single Ig and TIR domain containing | 185187 | 184363 | 185101 | 5, 29, 16 |
| 11 | SMCO4 | single-pass membrane protein with coiled-coil domains 4 | 166002 | 165325 | 166004 | 31, 34, 40 |
| 11 | SIRT3 | sirtuin 3 | 142082 | 177963 | 185627 | 38, 14, 34 |
| 11 | SSSCA1 | Sjogren syndrome/scleroderma autoantigen 1 | 173465 | 168056 | 176973 | 29, 28, 3 |
| 11 | SSH3 | slingshot protein phosphatase 3 | 172830 | 172932 | 172613 | 24, 27, 36 |
| 11 | SVIP | small VCP interacting protein | 198168 | 148935 | 255359 | 7, 28, 31 |
| 11 | SMTNL1 | smoothelin like 1 | 214872 | 134809 | 156587 | 25, 15, 19 |
| 11 | SCN2B | sodium voltage-gated channel beta subunit 2 | 149575 | 177098 | 160593 | 31, 15, 8 |
| 11 | SCN3B | sodium voltage-gated channel beta subunit 3 | 166257 | 023171 | 166261 | 8, 1, 26 |
| 11 | SCN4B | sodium voltage-gated channel beta subunit 4 | 177098 | 137648 | 149575 | 8, 42, 24 |
| 11 | SLC1A2 | solute carrier family 1 member 2 | 110436 | 026508 | 149090 | 8, 26, 3 |
| 11 | SLC15A3 | solute carrier family 15 member 3 | 110446 | 006118 | 013725 | 1, 10, 41 |
| 11 | SLC17A6 | solute carrier family 17 member 6 | 091664 | 171714 | 183161 | 8, 28, 17 |
| 11 | SLC22A10 | solute carrier family 22 member 10 | 184999 | 197658 | 196600 | 19, |
| 11 | SLC22A11 | solute carrier family 22 member 11 | 168065 | 162302 | 197891 | 32, 17, 8 |
| 11 | SLC22A12 | solute carrier family 22 member 12 | 197891 | 168065 | 110076 | 17, |
| 11 | SLC22A18 | solute carrier family 22 member 18 | 110628 | 254827 | 181649 | 16, 28, 26 |
| 11 | SLC22A18AS | solute carrier family 22 member 18 antisense | 254827 | 129757 | 110628 | 16, 6, 35 |
| 11 | SLC22A24 | solute carrier family 22 member 24 | 197658 | 149452 | 184999 | |
| 11 | SLC22A25 | solute carrier family 22 member 25 | 196600 | 184999 | 149742 | 19, 15, |
| 11 | SLC22A6 | solute carrier family 22 member 6 | 197901 | 168539 | 149452 | 17, 8, 25 |
| 11 | SLC22A8 | solute carrier family 22 member 8 | 149452 | 197901 | 197658 | 17, 14, 8 |
| 11 | SLC22A9 | solute carrier family 22 member 9 | 149742 | 196600 | 168004 | 19, |
| 11 | SLC25A22 | solute carrier family 25 member 22 | 177542 | 184524 | 274897 | 3, 26, 8 |
| 11 | SLC25A45 | solute carrier family 25 member 45 | 162241 | 173825 | 126391 | 36, 9, 29 |
| 11 | SLC29A2 | solute carrier family 29 member 2 | 174669 | 174684 | 174576 | 9, 40, 33 |
| 11 | SLC3A2 | solute carrier family 3 member 2 | 168003 | 133316 | 168539 | 13, 3, 4 |
| 11 | SLC35C1 | solute carrier family 35 member C1 | 181830 | 175264 | 121671 | 1, 40, 16 |
| 11 | SLC35F2 | solute carrier family 35 member F2 | 110660 | 170290 | 179331 | 45, 33, 24 |
| 11 | SLC36A4 | solute carrier family 36 member 4 | 180773 | 134640 | 165325 | 1, 9, 12 |
| 11 | SLC37A2 | solute carrier family 37 member 2 | 134955 | 149548 | 150433 | 24, 36, 2 |
| 11 | SLC37A4 | solute carrier family 37 member 4 | 137700 | 196655 | 149428 | 17, 39, 42 |
| 11 | SLC39A13 | solute carrier family 39 member 13 | 165915 | 066336 | 165916 | 13, 9, 31 |
| 11 | SLC43A1 | solute carrier family 43 member 1 | 149150 | 186907 | 134809 | 21, 41, 16 |
| 11 | SLC43A3 | solute carrier family 43 member 3 | 134802 | 254979 | 186907 | 7, 5, 42 |
| 11 | SLC5A12 | solute carrier family 5 member 12 | 148942 | 169550 | 176971 | 17, 12, 19 |
| 11 | SLC6A5 | solute carrier family 6 member 5 | 165970 | 185238 | 165973 | 38, |
| 11 | SLCO2B1 | solute carrier organic anion transporter family member 2B1 | 137491 | 171561 | 261594 | 10, 8, 42 |
| 11 | SORL1 | sortilin related receptor 1 | 137642 | 109929 | 259571 | 22, 5, 31 |
| 11 | SNX15 | sorting nexin 15 | 110025 | 273003 | 168061 | 9, 1, 43 |
| 11 | SNX19 | sorting nexin 19 | 120451 | 166106 | 182667 | 22, 14, 6 |
| 11 | SNX32 | sorting nexin 32 | 172803 | 172757 | 172732 | |
| 11 | SPTBN2 | spectrin beta, non-erythrocytic 2 | 173898 | 173914 | 173715 | 18, 14, 30 |
| 11 | SPDYC | speedy/RINGO cell cycle regulator family member C | 204710 | 149792 | 014216 | 34, 32, |
| 11 | SPA17 | sperm autoantigenic protein 17 | 064199 | 110013 | 154146 | 2, 38, 13 |
| 11 | SPATA19 | spermatogenesis associated 19 | 166118 | 183715 | 080854 | 38, 32, |
| 11 | SMPD1 | sphingomyelin phosphodiesterase 1 | 166311 | 170955 | 166313 | 29, 6, 26 |
| 11 | SPI1 | Spi-1 proto-oncogene | 066336 | 134571 | 165915 | 41, 22, 5 |
| 11 | SF1 | splicing factor 1 | 168066 | 068976 | 168067 | 2, 22, 12 |
| 11 | SF3B2 | splicing factor 3b subunit 2 | 087365 | 175229 | 175115 | |
| 11 | SPON1 | spondin 1 | 262655 | 197601 | 133818 | 27, 43, 44 |
| 11 | SPTY2D1 | SPT2 chromatin protein domain containing 1 | 179119 | 247595 | 151117 | 3, 1, 42 |
| 11 | SPTY2D1-AS1 | SPTY2D1 antisense RNA 1 | 247595 | 151116 | 179119 | 38, 33, |
| 11 | SART1 | squamous cell carcinoma antigen recognized by T-cells 1 | 175467 | 175513 | 175376 | 18, 1, 19 |
| 11 | SRPRA | SRP receptor alpha subunit | 182934 | 197798 | 110074 | 6, 23, 39 |
| 11 | SOX6 | SRY-box6 | 110693 | 188487 | 110696 | 1, 43, 17 |
| 11 | ST3GAL4 | ST3 beta-galactoside alpha-2,3-sialyltransferase 4 | 110080 | 110063 | 149571 | 37, 32, 9 |
| 11 | STARD10 | StAR related lipid transfer domain containing 10 | 214530 | 186635 | 168010 | 29, 16, 38 |
| 11 | SC5D | sterol-C5-desaturase | 109929 | 109927 | 137642 | 26, 11, 4 |
| 11 | STIP1 | stress induced phosphoprotein 1 | 168439 | 126500 | 149781 | |
| 11 | STIM1 | stromal interaction molecule 1 | 167323 | 177105 | 167325 | 41, 39, 26 |
| 11 | SSRP1 | structure specific recognition protein 1 | 149136 | 149115 | 109991 | 9, 21, 41 |
| 11 | STT3A | STT3A, catalytic subunit of the oligosaccharyltransferase complex | 134910 | 149547 | 149554 | 30, 24, 13 |
| 11 | SDHAF2 | succinate dehydrogenase complex assembly factor 2 | 167985 | 256591 | 162148 | 30, 9, 29 |
| 11 | SDHD | succinate dehydrogenase complex subunit D | 204370 | 150779 | 255292 | 34, 4, 7 |
| 11 | ST14 | suppression of tumorigenicity 14 | 149418 | 084234 | 196323 | 16, 37, 40 |
| 11 | ST5 | suppression of tumorigenicity 5 | 166444 | 166441 | 166452 | |
| 11 | SWAP70 | SWAP switching B-cell complex subunit 70 | 133789 | 166483 | 133812 | 41, 21, 18 |
| 11 | SYT12 | synaptotagmin 12 | 173227 | 173237 | 173156 | 29, 6, 33 |
| 11 | SYT13 | synaptotagmin 13 | 019505 | 019485 | 175264 | 3, 11, 28 |
| 11 | SYT7 | synaptotagmin 7 | 011347 | 204950 | 134780 | |
| 11 | SYT8 | synaptotagmin 8 | 149043 | 117984 | 130598 | 32, 4, 23 |
| 11 | SYT9 | synaptotagmin 9 | 170743 | 170748 | 183801 | 31, 26, 24 |
| 11 | SYTL2 | synaptotagmin like 2 | 137501 | 179071 | 150676 | 1, 23, 3 |
| 11 | SYVN1 | synoviolin 1 | 162298 | 149806 | 149792 | 22, 38, 35 |
| 11 | STX3 | syntaxin 3 | 166900 | 172289 | 166902 | 29, 37, 35 |
| 11 | STX5 | syntaxin 5 | 162236 | 162231 | 283268 | 33, 36, 18 |
| 11 | TNKS1BP1 | tankyrase 1 binding protein 1 | 149115 | 134817 | 149136 | 26, 40, 23 |
| 11 | TAF10 | TATA-box binding protein associated factor 10 | 166337 | 166333 | 166340 | 34, 27, 17 |
| 11 | TAF6L | TATA-box binding protein associated factor 6 like | 162227 | 168002 | 168569 | 29, 36, 20 |
| 11 | TAF1D | TATA-box binding protein associated factor, RNA polymerase I subunit D | 166012 | 166004 | 182919 | 13, 4, 7 |
| 11 | TBC1D10C | TBC1 domain family member 10C | 175463 | 172531 | 172508 | 41, 5, 22 |
| 11 | TBX10 | T-box 10 | 167800 | 167799 | 132744 | 16, 22, |
| 11 | TCIRG1 | T-cell immune regulator 1, ATPase H+ transporting V0 subunit a3 | 110719 | 110717 | 110721 | 7, 26, 20 |
| 11 | TCP11L1 | t-complex 11 like 1 | 176148 | 121690 | 280550 | 1, 35, 8 |
| 11 | TEAD1 | TEA domain transcription factor 1 | 187079 | 197702 | 189431 | 29, 31, 12 |
| 11 | TECTA | tectorin alpha | 109927 | 154114 | 109929 | 24, 5, 38 |
| 11 | TENM4 | teneurin transmembrane protein 4 | 149256 | 137513 | 182103 | 40, 29, 24 |
| 11 | TUT1 | terminal uridylyl transferase 1, U6 snRNA-specific | 149016 | 255508 | 149480 | 43, 39, 21 |
| 11 | TEX12 | testis expressed 12 | 150783 | 150782 | 197580 | 38, 25, |
| 11 | TEX40 | testis expressed 40 | 219435 | 182450 | 173153 | 38, 32, 14 |
| 11 | TESMIN | testis expressed metallothionein like protein | 132749 | 069482 | 110090 | 38, |
| 11 | TSGA10IP | testis specific 10 interacting protein | 175513 | 175550 | 175467 | 38, 8, |
| 11 | TSPAN18 | tetraspanin 18 | 157570 | 085117 | 175274 | 43, 15, 2 |
| 11 | TSPAN32 | tetraspanin 32 | 064201 | 110665 | 110651 | 5, 36, 13 |
| 11 | TSPAN4 | tetraspanin 4 | 214063 | 177700 | 177830 | 22, 1, 13 |
| 11 | TTC12 | tetratricopeptide repeat domain 12 | 149292 | 149294 | 170209 | 38, 18, 31 |
| 11 | TTC17 | tetratricopeptide repeat domain 17 | 052841 | 166181 | 149084 | 24, 34, 13 |
| 11 | TTC36 | tetratricopeptide repeat domain 36 | 172425 | 118058 | 149582 | 17, 19, 14 |
| 11 | TTC9C | tetratricopeptide repeat domain 9C | 162222 | 214753 | 185670 | 15, 22, 41 |
| 11 | THAP12 | THAP domain containing 12 | 137492 | 085741 | 179240 | 27, 4, 22 |
| 11 | TMX2 | thioredoxin related transmembrane protein 2 | 213593 | 156603 | 254462 | 30, 24, 40 |
| 11 | THY1 | Thy-1 cell surface antigen | 154096 | 036672 | 110400 | 8, 32, 26 |
| 11 | THYN1 | thymocyte nuclear protein 1 | 151500 | 151502 | 151498 | 5, 40, 4 |
| 11 | THRSP | thyroid hormone responsive | 151365 | 259112 | 151366 | 1, 35, 19 |
| 11 | TIGD3 | tigger transposable element derived 3 | 173825 | 133884 | 162241 | 29, 5, 14 |
| 11 | TIRAP | TIR domain containing adaptor protein | 150455 | 110074 | 110063 | 42, 19, 34 |
| 11 | TMEM9B | TMEM9 domain family member B | 175348 | 176009 | 175352 | 29, 1, 31 |
| 11 | TMPRSS4-AS1 | TMPRSS4 antisense RNA 1 | 255274 | 110324 | 279906 | |
| 11 | TMX2-CTNND1 | TMX2-CTNND1 readthrough (NMD candidate) | 254462 | 213593 | 211450 | |
| 11 | TRAF6 | TNF receptor associated factor 6 | 175104 | 135362 | 166349 | 29, 1, 11 |
| 11 | TOLLIP | toll interacting protein | 078902 | 117983 | 174672 | 34, 8, 1 |
| 11 | TRAPPC4 | trafficking protein particle complex 4 | 196655 | 118181 | 137700 | 29, 24, 30 |
| 11 | TALDO1 | transaldolase 1 | 177156 | 177042 | 177225 | 13, 34, 1 |
| 11 | TCN1 | transcobalamin 1 | 134827 | 134812 | 149507 | 34, 18, 30 |
| 11 | TBRG1 | transforming growth factor beta regulator 1 | 154144 | 154143 | 110013 | 1, 8, 22 |
| 11 | TAGLN | transgelin | 149591 | 149577 | 160613 | 26, 12, 7 |
| 11 | TRPC6 | transient receptor potential cation channel subfamily C member 6 | 137672 | 082175 | 187151 | 15, 32, 45 |
| 11 | TRPM5 | transient receptor potential cation channel subfamily M member 5 | 070985 | 184281 | 053918 | 16, 5, 23 |
| 11 | TIMM10 | translocase of inner mitochondrial membrane 10 | 134809 | 149150 | 214872 | 33, 12, 14 |
| 11 | TIMM10B | translocase of inner mitochondrial membrane 10B | 132286 | 132254 | 179532 | |
| 11 | TIMM8B | translocase of inner mitochondrial membrane 8 homolog B | 150779 | 150776 | 204370 | 11, 15, 25 |
| 11 | TM7SF2 | transmembrane 7 superfamily member 2 | 149809 | 149823 | 174276 | 27, 33, 5 |
| 11 | TMPRSS13 | transmembrane protease, serine 13 | 137747 | 137726 | 110324 | 1, 23, 24 |
| 11 | TMPRSS4 | transmembrane protease, serine 4 | 137648 | 279906 | 177098 | 13, 24, 16 |
| 11 | TMPRSS5 | transmembrane protease, serine 5 | 166682 | 149295 | 086827 | 8, 35, 6 |
| 11 | TMEM109 | transmembrane protein 109 | 110108 | 110107 | 006118 | 35, 31, 22 |
| 11 | TMEM123 | transmembrane protein 123 | 152558 | 110330 | 137673 | 42, 22, 4 |
| 11 | TMEM126A | transmembrane protein 126A | 171202 | 171204 | 137504 | 29, 15, 31 |
| 11 | TMEM126B | transmembrane protein 126B | 171204 | 150672 | 171202 | 4, 29, 7 |
| 11 | TMEM132A | transmembrane protein 132A | 006118 | 110108 | 110446 | |
| 11 | TMEM133 | transmembrane protein 133 | 170647 | 165895 | 082175 | 2, 11, 22 |
| 11 | TMEM134 | transmembrane protein 134 | 172663 | 175544 | 110711 | 39, 22, 23 |
| 11 | TMEM135 | transmembrane protein 135 | 166575 | 174804 | 123892 | 45, 31, 34 |
| 11 | TMEM136 | transmembrane protein 136 | 181264 | 137709 | 196914 | 26, 42, 8 |
| 11 | TMEM138 | transmembrane protein 138 | 149483 | 162144 | 187049 | 31, 40, 42 |
| 11 | TMEM151A | transmembrane protein 151A | 179292 | 174851 | 174807 | 26, 14, 8 |
| 11 | TMEM179B | transmembrane protein 179B | 185475 | 168569 | 162231 | 34, 29, 15 |
| 11 | TMEM216 | transmembrane protein 216 | 187049 | 149483 | 149532 | 30, 6, 4 |
| 11 | TMEM218 | transmembrane protein 218 | 150433 | 134955 | 165495 | 1, 17, 9 |
| 11 | TMEM223 | transmembrane protein 223 | 168569 | 162227 | 185475 | 41, 34, 22 |
| 11 | TMEM225 | transmembrane protein 225 | 204300 | 196099 | 181518 | 38, |
| 11 | TMEM25 | transmembrane protein 25 | 149582 | 172425 | 118096 | 2, 8, 39 |
| 11 | TMEM258 | transmembrane protein 258 | 134825 | 124920 | 168496 | 12, |
| 11 | TMEM262 | transmembrane protein 262 | 187066 | 162300 | 149823 | 29, 36, 16 |
| 11 | TMEM41B | transmembrane protein 41B | 166471 | 184014 | 205339 | 11, 7, 39 |
| 11 | TMEM45B | transmembrane protein 45B | 151715 | 043039 | 170322 | 16, 42, 9 |
| 11 | TMEM80 | transmembrane protein 80 | 177042 | 177106 | 177156 | 1, 32, 16 |
| 11 | TMEM86A | transmembrane protein 86A | 151117 | 179119 | 179057 | 10, 23, 17 |
| 11 | TREH | trehalase | 118094 | 019144 | 110367 | 17, 38, 16 |
| 11 | TRIM6-TRIM34 | TRIM6-TRIM34 readthrough | 258588 | 121236 | 258659 | 34, 17, 32 |
| 11 | TKFC | triokinase and FMN cyclase | 149476 | 167986 | 162144 | 38, |
| 11 | TRIM21 | tripartite motif containing 21 | 132109 | 221996 | 181963 | |
| 11 | TRIM22 | tripartite motif containing 22 | 132274 | 132256 | 181023 | 43, 36, 26 |
| 11 | TRIM29 | tripartite motif containing 29 | 137699 | 110400 | 184232 | 24, 18, 35 |
| 11 | TRIM3 | tripartite motif containing 3 | 110171 | 110169 | 132254 | 8, 23, 34 |
| 11 | TRIM34 | tripartite motif containing 34 | 258659 | 258588 | 132256 | |
| 11 | TRIM44 | tripartite motif containing 44 | 166326 | 179431 | 179241 | 29, 41, 44 |
| 11 | TRIM48 | tripartite motif containing 48 | 150244 | 221840 | 181961 | 35, 32, 44 |
| 11 | TRIM49 | tripartite motif containing 49 | 168930 | 214414 | 189253 | |
| 11 | TRIM49B | tripartite motif containing 49B | 182053 | 237388 | 214891 | 8, |
| 11 | TRIM49C | tripartite motif containing 49C | 204449 | 204450 | 255009 | |
| 11 | TRIM49D1 | tripartite motif containing 49D1 | 223417 | 189253 | 233802 | |
| 11 | TRIM49D2 | tripartite motif containing 49D2 | 233802 | 223417 | 204450 | |
| 11 | TRIM5 | tripartite motif containing 5 | 132256 | 258659 | 132274 | 39, 4, 44 |
| 11 | TRIM6 | tripartite motif containing 6 | 121236 | 187747 | 258588 | 34, 17, 32 |
| 11 | TRIM64 | tripartite motif containing 64 | 204450 | 233802 | 204449 | 44, |
| 11 | TRIM64B | tripartite motif containing 64B | 189253 | 168930 | 223417 | |
| 11 | TRIM64C | tripartite motif containing 64C | 214891 | 182053 | 086205 | |
| 11 | TRIM66 | tripartite motif containing 66 | 166436 | 130413 | 166441 | 29, 22, 26 |
| 11 | TRIM68 | tripartite motif containing 68 | 167333 | 232268 | 197428 | |
| 11 | TRIM77 | tripartite motif containing 77 | 214414 | 086991 | 168930 | |
| 11 | TRIM51 | tripartite motif-containing 51 | 124900 | 205029 | 187612 | |
| 11 | TPP1 | tripeptidyl peptidase 1 | 166340 | 166337 | 166341 | 36, 39, 42 |
| 11 | TRMT112 | tRNA methyltransferase 11-2 homolog (S. cerevisiae) | 173113 | 173153 | 126432 | 29, 15, 7 |
| 11 | TRPT1 | tRNA phosphotransferase 1 | 149743 | 149781 | 149761 | 34, 40, 22 |
| 11 | TPBGL | trophoblast glycoprotein-like | 261594 | 137491 | 137486 | 14, 35, 28 |
| 11 | TNNI2 | troponin I2, fast skeletal type | 130598 | 149043 | 130592 | 25, 30, 24 |
| 11 | TNNT3 | troponin T3, fast skeletal type | 130595 | 130592 | 214026 | 25, 18, 15 |
| 11 | TPH1 | tryptophan hydroxylase 1 | 129167 | 129158 | 166788 | 31, 20, 14 |
| 11 | TSKU | tsukushi, small leucine rich proteoglycan | 182704 | 137507 | 078124 | 24, 23, 42 |
| 11 | TUB | tubby bipartite transcription factor | 166402 | 175390 | 166405 | 29, 14, 18 |
| 11 | TBCEL | tubulin folding cofactor E like | 154114 | 149403 | 109927 | 29, 9, 13 |
| 11 | TP53I11 | tumor protein p53 inducible protein 11 | 175274 | 157570 | 019485 | 34, 3, 24 |
| 11 | TP53AIP1 | tumor protein p53 regulated apoptosis inducing protein 1 | 120471 | 174370 | 134909 | 31, 4, 24 |
| 11 | TSSC4 | tumor suppressing subtransferable candidate 4 | 184281 | 110651 | 070985 | 34, 29, 21 |
| 11 | TSG101 | tumor susceptibility 101 | 074319 | 166800 | 151116 | 31, 5, 7 |
| 11 | TPCN2 | two pore segment channel 2 | 162341 | 172935 | 172927 | 18, 14, 41 |
| 11 | TYR | tyrosinase | 077498 | 168959 | 086991 | 35, 40, 11 |
| 11 | TH | tyrosine hydroxylase | 180176 | 254647 | 183734 | 26, 24, 2 |
| 11 | UBQLN3 | ubiquilin 3 | 175520 | 181609 | 175518 | 38, 8, |
| 11 | UBQLNL | ubiquilin-like | 175518 | 175520 | 181616 | 38, 45, 44 |
| 11 | UQCC3 | ubiquinol-cytochrome c reductase complex assembly factor 3 | 204922 | 214756 | 162191 | 31, 14, 3 |
| 11 | UBASH3B | ubiquitin associated and SH3 domain containing B | 154127 | 259571 | 109943 | 36, 17, 45 |
| 11 | UBE2L6 | ubiquitin conjugating enzyme E2 L6 | 156587 | 214872 | 149131 | 30, 11, 41 |
| 11 | USP2 | ubiquitin specific peptidase 2 | 036672 | 235718 | 154096 | 38, 29, 21 |
| 11 | USP28 | ubiquitin specific peptidase 28 | 048028 | 228607 | 149305 | 9, 15, 12 |
| 11 | USP35 | ubiquitin specific peptidase 35 | 118369 | 188997 | 033327 | 7, 28, 6 |
| 11 | USP47 | ubiquitin specific peptidase 47 | 170242 | 254598 | 050165 | 11, 13, 43 |
| 11 | UBE4A | ubiquitination factor E4A | 110344 | 160654 | 167283 | 11, 27, 23 |
| 11 | UBXN1 | UBX domain protein 1 | 162191 | 204922 | 177363 | 41, 18, 21 |
| 11 | B3GNT6 | UDP-GlcNAc:betaGal beta-1,3-N-acetylglucosaminyltransferase 6 | 198488 | 078124 | 149260 | 42, 24, 16 |
| 11 | UEVLD | UEV and lactate/malate dehyrogenase domains | 151116 | 074319 | 247595 | 24, 13, 22 |
| 11 | UNC93B1 | unc-93 homolog B1 (C. elegans) | 110057 | 132746 | 006534 | 3, 41, 22 |
| 11 | UCP2 | uncoupling protein 2 | 175567 | 187726 | 175564 | 41, 36, 21 |
| 11 | UCP3 | uncoupling protein 3 | 175564 | 175567 | 168014 | 25, 15, 45 |
| 11 | UBTFL1 | upstream binding transcription factor, RNA polymerase I-like 1 | 255009 | 204449 | 077616 | |
| 11 | UPK2 | uroplakin 2 | 110375 | 186174 | 176302 | 4, 14, 12 |
| 11 | USH1C | USH1 protein network component harmonin | 006611 | 006071 | 188162 | 9, 16, 17 |
| 11 | UVRAG | UV radiation resistance associated | 198382 | 062282 | 085741 | 22, 1, 11 |
| 11 | VEGFB | vascular endothelial growth factor B | 173511 | 110011 | 173486 | |
| 11 | VWA5A | von Willebrand factor A domain containing 5A | 110002 | 182634 | 197309 | 42, 11, 9 |
| 11 | VWCE | von Willebrand factor C and EGF domains | 167992 | 256713 | 167986 | 21, 19, 14 |
| 11 | VPS11 | VPS11, CORVET/HOPS core subunit | 160695 | 149428 | 256269 | 43, 24, 39 |
| 11 | VPS26B | VPS26, retromer complex component B | 151502 | 151503 | 151500 | 34, 33, 6 |
| 11 | VPS37C | VPS37C, ESCRT-I subunit | 167987 | 110448 | 229859 | 1, 33, 38 |
| 11 | VPS51 | VPS51, GARP complex subunit | 149823 | 187066 | 149809 | 41, 9, 31 |
| 11 | VSIG2 | V-set and immunoglobulin domain containing 2 | 019102 | 154146 | 149564 | 3, 37, 16 |
| 11 | VSTM5 | V-set and transmembrane domain containing 5 | 214376 | 042429 | 181333 | 20, |
| 11 | WDR74 | WD repeat domain 74 | 133316 | 283268 | 168003 | |
| 11 | WEE1 | WEE1 G2 checkpoint kinase | 166483 | 166478 | 133789 | 4, 15, 7 |
| 11 | WT1 | Wilms tumor 1 | 184937 | 049449 | 149100 | 38, 34, 27 |
| 11 | WNT11 | Wnt family member 11 | 085741 | 198382 | 137492 | |
| 11 | XRRA1 | X-ray radiation resistance associated 1 | 166435 | 166439 | 118363 | 29, 38, 14 |
| 11 | YAP1 | Yes associated protein 1 | 137693 | 137691 | 023445 | 29, 11, 4 |
| 11 | YIF1A | Yip1 interacting factor homolog A, membrane trafficking protein | 174851 | 174871 | 179292 | 24, 28, 3 |
| 11 | YPEL4 | yippee like 4 | 166793 | 149131 | 172409 | 8, 2, 40 |
| 11 | ZFP91 | ZFP91 zinc finger protein | 186660 | 110031 | 255073 | 22, 18, 11 |
| 11 | ZFP91-CNTF | ZFP91-CNTF readthrough (NMD candidate) | 255073 | 186660 | 242689 | |
| 11 | ZBTB16 | zinc finger and BTB domain containing 16 | 109906 | 166736 | 166741 | 1, 26, 36 |
| 11 | ZBTB3 | zinc finger and BTB domain containing 3 | 185670 | 162222 | 168002 | 39, 38, 15 |
| 11 | ZBTB44 | zinc finger and BTB domain containing 44 | 196323 | 149418 | 134917 | 11, 22, 25 |
| 11 | ZBED5 | zinc finger BED-type containing 5 | 236287 | 110321 | 110328 | 42, 39, 8 |
| 11 | ZC3H12C | zinc finger CCCH-type containing 12C | 149289 | 185742 | 137710 | 26, 17, 44 |
| 11 | ZDHHC13 | zinc finger DHHC-type containing 13 | 177054 | 183695 | 129170 | 24, 4, 45 |
| 11 | ZDHHC24 | zinc finger DHHC-type containing 24 | 174165 | 174483 | 248746 | 41, 26, 37 |
| 11 | ZDHHC5 | zinc finger DHHC-type containing 5 | 156599 | 172409 | 156603 | 18, 22, 40 |
| 11 | ZNHIT2 | zinc finger HIT-type containing 2 | 174276 | 149809 | 149806 | 38, 17, 16 |
| 11 | ZNF143 | zinc finger protein 143 | 166478 | 205339 | 166483 | 31, 39, 38 |
| 11 | ZNF195 | zinc finger protein 195 | 005801 | 184350 | 167311 | 13, 37, 4 |
| 11 | ZNF202 | zinc finger protein 202 | 166261 | 166257 | 221931 | 29, 11, 42 |
| 11 | ZNF214 | zinc finger protein 214 | 149050 | 149054 | 158077 | |
| 11 | ZNF215 | zinc finger protein 215 | 149054 | 178358 | 149050 | 22, 3, 45 |
| 11 | ZNF408 | zinc finger protein 408 | 175213 | 175220 | 180210 | 39, 34, 43 |
| 11 | ZFPL1 | zinc finger protein like 1 | 162300 | 146670 | 187066 | 29, 36, 16 |
| 11 | ZP1 | zona pellucida glycoprotein 1 | 149506 | 183134 | 110107 | 39, 38, 23 |
| 11 | ZPR1 | ZPR1 zinc finger | 109917 | 137656 | 110243 | |
| 11 | ZW10 | zw10 kinetochore protein | 086827 | 166682 | 228607 | 24, 33, 6 |

Table 6 provides the gene information for the genes found on Chromosomes 12-14.

TABLE 6
Genes on Chromosomes 12-14

| Chr. | Gene Symbol | Gene Name | ENSG | Up ENSG | Down ENSG | Tissue Code |
|---|---|---|---|---|---|---|
| 12 | OAS1 | 2′-5′-oligoadenylate synthetase 1 | 089127 | 089169 | 111331 | 41, 16, 4 |
| 12 | OAS2 | 2′-5′-oligoadenylate synthetase 2 | 111335 | 111331 | 135144 | 42, 22, 21 |
| 12 | OAS3 | 2′-5′-oligoadenylate synthetase 3 | 111331 | 089127 | 111335 | |
| 12 | OASL | 2′-5′-oligoadenylate synthetase like | 135114 | 157895 | 089041 | 9, 13, 1 |
| 12 | OGFOD2 | 2-oxoglutarate and iron dependent oxygenase domain containing 2 | 111325 | 150967 | 182196 | 34, 6, 9 |
| 12 | HPD | 4-hydroxyphenylpyruvate dioxygenase | 158104 | 139718 | 110801 | 19, 9, 14 |
| 12 | NT5DC3 | 5′-nucleotidase domain containing 3 | 111696 | 136011 | 166598 | 35, 16, 3 |
| 12 | AACS | acetoacetyl-CoA synthetase | 081760 | 184992 | 139364 | 24, 42, 21 |
| 12 | ACACB | acetyl-CoA carboxylase beta | 076555 | 076248 | 139445 | 1, 23, 26 |
| 12 | ASCL1 | achaete-scute family bHLH transcription factor 1 | 139352 | 171759 | 179088 | 20, 8, 26 |
| 12 | ASCL4 | achaete-scute family bHLH transcription factor 4 | 187855 | 110851 | 075035 | 15, |
| 12 | ASIC1 | acid sensing ion channel subunit 1 | 110881 | 161800 | 066117 | 8, 14, 6 |
| 12 | ANP32D | acidic nuclear phosphoprotein 32 family member D | 139223 | 167528 | 177627 | |
| 12 | ACRBP | acrosin binding protein | 111644 | 184574 | 111653 | 38, 36, 8 |
| 12 | ARPC3 | actin related protein 2/3 complex subunit 3 | 111229 | 196510 | 111231 | 23, 29, 43 |
| 12 | ATF1 | activating transcription factor 1 | 123268 | 066084 | 186452 | 34, 18, 22 |
| 12 | ATF7 | activating transcription factor 7 | 170653 | 139574 | 135390 | 18, 34, 30 |
| 12 | ATF7IP | activating transcription factor 7 interacting protein | 171681 | 273079 | 121316 | 42, 38, 9 |
| 12 | AICDA | activation induced cytidine deaminase | 111732 | 166523 | 197614 | 41, 21, 22 |
| 12 | ACVRL1 | activin A receptor like type 1 | 139567 | 167612 | 135503 | 43, 45, 23 |
| 12 | ACVR1B | activin A receptor type 1B | 135503 | 139567 | 161835 | 29, 18, 30 |
| 12 | ACAD10 | acyl-CoA dehydrogenase family member 10 | 111271 | 089234 | 257767 | 9, 1, 17 |
| 12 | ACADS | acyl-CoA dehydrogenase, C-2 to C-3 short chain | 122971 | 175970 | 157837 | 3, 26, 38 |
| 12 | ACSM4 | acyl-CoA synthetase medium-chain family member 4 | 215009 | 139197 | 177675 | 12, 38, |
| 12 | ACSS3 | acyl-CoA synthetase short-chain family member 3 | 111058 | 111052 | 139220 | 10, 38, 30 |
| 12 | ADAMTS20 | ADAM metallopeptidase with thrombospondin type 1 motif 20 | 173157 | 139174 | 129317 | 24, 10, 14 |
| 12 | APPL2 | adaptor protein, phosphotyrosine interacting with PH domain | 136044 | 136051 | 235162 | 29, 11, 31 |
| | | and leucine zipper 2 | | | | |
| 12 | ADCY6 | adenylate cyclase 6 | 174233 | 257987 | 167535 | 6, 18, 12 |
| 12 | ADGRD1 | adhesion G protein-coupled receptor D1 | 111452 | 132341 | 061936 | 1, 38, 32 |
| 12 | AMIGO2 | adhesion molecule with Ig like domain 2 | 139211 | 139209 | 179715 | 45, 26, 31 |
| 12 | ADIPOR2 | adiponectin receptor 2 | 006831 | 171823 | 151062 | |
| 12 | ARF3 | ADP ribosylation factor 3 | 134287 | 255863 | 169884 | 8, 23, 38 |
| 12 | ARL1 | ADP ribosylation factor like GTPase 1 | 120805 | 120800 | 166211 | |
| 12 | ARL6IP4 | ADP ribosylation factor like GTPase 6 interacting protein 4 | 182196 | 111325 | 090975 | 31, 6, 28 |
| 12 | ART4 | ADP-ribosyltransferase 4 (Dombrock blood group) | 111339 | 179256 | 111341 | |
| 12 | AVIL | advillin | 135407 | 123297 | 175215 | 26, 25, 27 |
| 12 | AEBP2 | AE binding protein 2 | 139154 | 052126 | 172572 | 32, 40, 35 |
| 12 | AKAP3 | A-kinase anchoring protein 3 | 111254 | 272921 | 139180 | 38, 45, 36 |
| 12 | AAAS | aladin WD repeat nucleoporin | 094914 | 139637 | 170374 | 3, 9, 37 |
| 12 | ALDH1L2 | aldehyde dehydrogenase 1 family member L2 | 136010 | 151131 | 136051 | 1, 6, 42 |
| 12 | ALDH2 | aldehyde dehydrogenase 2 family (mitochondrial) | 111275 | 257767 | 089022 | 1, 19, 11 |
| 12 | ALG10 | ALG10, alpha-1,2-glucosyltransferase | 139133 | 110975 | 175548 | 29, 9, 23 |
| 12 | ALG10B | ALG10B, alpha-1,2-glucosyltransferase | 175548 | 139133 | 139117 | 4, 22, 29 |
| 12 | ALKBH2 | alkB homolog 2, alpha-ketoglutarate dependent dioxygenase | 189046 | 135093 | 076248 | 43, 3, 40 |
| 12 | A2M | alpha-2-macroglobulin | 175899 | 139187 | 126838 | 42, 19, 1 |
| 12 | A2ML1 | alpha-2-macroglobulin like 1 | 166535 | 166532 | 111752 | 24, 30, 13 |
| 12 | ALX1 | ALX homeobox 1 | 180318 | 133640 | 198774 | 35, 19, 23 |
| 12 | AMDHD1 | amidohydrolase domain containing 1 | 139344 | 165972 | 084110 | 2, 17, 19 |
| 12 | ANAPC5 | anaphase promoting complex subunit 5 | 089053 | 110931 | 170633 | 3, 1, 43 |
| 12 | ANAPC7 | anaphase promoting complex subunit 7 | 196510 | 174437 | 111229 | 29, 26, 2 |
| 12 | ANKLE2 | ankyrin repeat and LEM domain containing 2 | 176915 | 247077 | 090615 | 38, 22, 24 |
| 12 | ASB8 | ankyrin repeat and SOCS box containing 8 | 177981 | 152556 | 177875 | 18, 29, 25 |
| 12 | ANKS1B | ankyrin repeat and sterile alpha motif domain containing 1B | 185046 | 120868 | 180219 | 8, 11, 26 |
| 12 | ANKRD13A | ankyrin repeat domain 13A | 076513 | 139436 | 174456 | 40, 22, 38 |
| 12 | ANKRD33 | ankyrin repeat domain 33 | 167612 | 196876 | 139567 | 32, 14, 8 |
| 12 | ANKRD52 | ankyrin repeat domain 52 | 139645 | 139540 | 135469 | 43, 26, 33 |
| 12 | ANO2 | anoctamin 2 | 047617 | 185652 | 110799 | 14, 36, 38 |
| 12 | ANO4 | anoctamin 4 | 151572 | 139354 | 256870 | 31, 8, 17 |
| 12 | ANO6 | anoctamin 6 | 177119 | 185610 | 189079 | 1, 11, 40 |
| 12 | ANHX | anomalous homeobox | 227059 | 090612 | 111291 | 38, |
| 12 | AMN1 | antagonist of mitotic exit network 1 homolog | 151743 | 139160 | 188375 | 13, 34, 8 |
| 12 | AMHR2 | anti-Mullerian hormone receptor type 2 | 135409 | 185591 | 205352 | 36, 38, 15 |
| 12 | APOBEC1 | apolipoprotein B mRNA editing enzyme catalytic subunit 1 | 111701 | 177575 | 184344 | 16, |
| 12 | APOF | apolipoprotein F | 175336 | 170581 | 111602 | 19, 23, |
| 12 | APOLD1 | apolipoprotein L domain containing 1 | 178878 | 111276 | 213782 | 42, 26, 28 |
| 12 | APAF1 | apoptotic peptidase activating factor 1 | 120868 | 166130 | 185046 | 29, 11, 22 |
| 12 | AQP2 | aquaporin 2 | 167580 | 135472 | 161798 | 17, 33, 9 |
| 12 | AQP5 | aquaporin 5 | 161798 | 167580 | 086159 | 37, 38, 14 |
| 12 | AQP6 | aquaporin 6 | 086159 | 161798 | 161800 | 17, 10, 35 |
| 12 | AGAP2 | ArfGAP with GTPase domain, ankyrin repeat and PH domain 2 | 135439 | 135506 | 135452 | 39, 34, 5 |
| 12 | RSRC2 | arginine and serine rich coiled-coil 2 | 111011 | 033030 | 184445 | 15, 4, 11 |
| 12 | AVPR1A | arginine vasopressin receptor 1A | 166148 | 111110 | 177990 | 17, 11, 29 |
| 12 | ACTR6 | ARP6 actin-related protein 6 homolog | 075089 | 111647 | 166153 | 3, 32, 4 |
| 12 | ARNTL2 | aryl hydrocarbon receptor nuclear translocator like 2 | 029153 | 211455 | 165935 | |
| 12 | ASUN | asunder, spermatogenesis regulator | 064102 | 123104 | 111790 | 7, 45, 4 |
| 12 | ATXN2 | ataxin 2 | 204842 | 111252 | 089234 | 18, 31, 45 |
| 12 | ATXN7L3B | ataxin 7 like 3B | 253719 | 072657 | 166006 | 42, 29, 39 |
| 12 | ABCB9 | ATP binding cassette subfamily B member 9 | 150967 | 139722 | 111325 | 9, 8, 3 |
| 12 | ABCC9 | ATP binding cassette subfamily C member 9 | 069431 | 121361 | 111726 | 11, 26, 42 |
| 12 | ABCD2 | ATP binding cassette subfamily D member 2 | 173208 | 139116 | 180116 | 30, 8, 42 |
| 12 | ATP5B | ATP synthase, H+ transporting, mitochondrial F1 complex, | 110955 | 076108 | 110958 | 21, 9, 40 |
| | | beta polypeptide | | | | |
| 12 | ATP5G2 | ATP synthase, H+ transporting, mitochondrial Fo complex | 135390 | 170653 | 012822 | 4, 34, 7 |
| | | subunit C2 (subunit 9) | | | | |
| 12 | ATP23 | ATP23 metallopeptidase and ATP synthase assembly factor homolog | 166896 | 175215 | 139263 | 22, 17, 15 |
| 12 | ATP6V0A2 | ATPase H+ transporting V0 subunit a2 | 185344 | 168778 | 197653 | 22, 1, 41 |
| 12 | ATP2B1 | ATPase plasma membrane Ca2+ transporting 1 | 070961 | 259075 | 197651 | 6, 22, 11 |
| 12 | ATP2A2 | ATPase sarcoplasmic/endoplasmic reticulum Ca2+ transporting 2 | 174437 | 122970 | 196510 | 25, 15, 26 |
| 12 | ARID2 | AT-rich interaction domain 2 | 189079 | 177119 | 139218 | 38, 40, 18 |
| 12 | ATN1 | atrophin 1 | 111676 | 111674 | 111678 | 40, 18, 4 |
| 12 | ATG101 | autophagy related 101 | 123395 | 123358 | 167767 | |
| 12 | BBS10 | Bardet-Biedl syndrome 10 | 179941 | 187109 | 091039 | 9, 42, 1 |
| 12 | BHLHE41 | basic helix-loop-helix family member e41 | 123095 | 123094 | 123096 | 29, 14, 22 |
| 12 | BCDIN3D | BCDIN3 domain containing RNA methyltransferase | 186666 | 167566 | 135472 | 41, 27, 9 |
| 12 | BCL7A | BCL tumor suppressor 7A | 110987 | 158023 | 175727 | 29, 22, 40 |
| 12 | BCL2L14 | BCL2 like 14 | 121380 | 139083 | 070018 | |
| 12 | BEST3 | bestrophin 3 | 127325 | 198812 | 127328 | 24, 26, 21 |
| 12 | B4GALNT1 | beta-1,4-N-acetyl-galactosaminyltransferase 1 | 135454 | 135502 | 135506 | 8, 6, 33 |
| 12 | B4GALNT3 | beta-1,4-N-acetyl-galactosaminyltransferase 3 | 139044 | 120647 | 171840 | 24, 38, 16 |
| 12 | BICD1 | BICD cargo adaptor 1 | 151746 | 174718 | 139132 | 27, 22, 1 |
| 12 | BICDL1 | BICD family like cargo adaptor 1 | 135127 | 279777 | 111737 | 26, 18, 31 |
| 12 | BLOC1S1 | biogenesis of lysosomal organelles complex 1 subunit 1 | 135441 | 258311 | 135437 | 41, 5, 19 |
| 12 | BORCS5 | BLOC-1 related complex subunit 5 | 165714 | 111261 | 111266 | |
| 12 | BCAT1 | branched chain amino acid transaminase 1 | 060982 | 134532 | 118308 | 12, 38, 45 |
| 12 | BRAP | BRCA1 associated protein | 089234 | 204842 | 111271 | 38, 13, 21 |
| 12 | BRI3BP | BRI3 binding protein | 184992 | 150990 | 081760 | 21, 22, 28 |
| 12 | BIN2 | bridging integrator 2 | 110934 | 170545 | 139610 | 36, 5, 39 |
| 12 | BAZ2A | bromodomain adjacent to zinc finger domain 2A | 076108 | 076067 | 110955 | 26, 43, 21 |
| 12 | BTBD11 | BTB domain containing 11 | 151136 | 008405 | 136045 | 29, 31, 40 |
| 12 | BTG1 | BTG anti-proliferation factor 1 | 133639 | 011465 | 205057 | 11, 28, 22 |
| 12 | C2CD5 | C2 calcium dependent domain containing 5 | 111731 | 111728 | 139163 | 11, 45, 42 |
| 12 | CALCOCO1 | calcium binding and coiled-coil domain 1 | 012822 | 135390 | 123364 | 39, 42, 8 |
| 12 | CABP1 | calcium binding protein 1 | 157782 | 167272 | 110917 | 14, 8, 16 |
| 12 | CRACR2A | calcium release activated channel regulator 2A | 130038 | 111218 | 111224 | |
| 12 | CACNA2D4 | calcium voltage-gated channel auxiliary subunit alpha2delta 4 | 151062 | 006831 | 166159 | 7, 36, 5 |
| 12 | CACNB3 | calcium voltage-gated channel auxiliary subunit beta 3 | 167535 | 174233 | 174243 | 43, 13, 8 |
| 12 | CACNA1C | calcium voltage-gated channel subunit alphal C | 151067 | 151065 | 004478 | 15, |
| 12 | CAMKK2 | calcium/calmodulin dependent protein kinase kinase 2 | 110931 | 135124 | 089053 | 8, 33, 18 |
| 12 | CAPS2 | calcyphosine 2 | 180881 | 166006 | 173401 | 11, 31, 10 |
| 12 | CLSTN3 | calsyntenin 3 | 139182 | 139194 | 139197 | 8, 43, 26 |
| 12 | CREBL2 | cAMP responsive element binding protein like 2 | 111269 | 111266 | 183150 | 4, 2, 11 |
| 12 | CASC1 | cancer susceptibility candidate 1 | 118307 | 118308 | 205707 | 38, 42, 31 |
| 12 | CNPY2 | canopy FGF signaling regulator 2 | 257727 | 144785 | 135473 | 3, 1, 28 |
| 12 | CLIP1 | CAP-Gly domain containing linker protein 1 | 130779 | 139719 | 033030 | 45, 40, 18 |
| 12 | CAPZA3 | capping actin protein of muscle Z-line alpha subunit 3 | 177938 | 139151 | 052126 | 38, 8, |
| 12 | CAPRIN2 | caprin family member 2 | 110888 | 133704 | 110900 | 11, 26, 10 |
| 12 | CHST11 | carbohydrate sulfotransferase 11 | 171310 | 255150 | 136052 | 22, 35, 5 |
| 12 | CPM | carboxypeptidase M | 135678 | 135679 | 111605 | |
| 12 | CRADD | CASP2 and RIPK1 domain containing adaptor with death domain | 169372 | 120833 | 136040 | |
| 12 | CNOT2 | CCR4-NOT transcription complex subunit 2 | 111596 | 166268 | 279530 | 11, 31, 15 |
| 12 | CD163 | CD163 molecule | 177575 | 177675 | 111701 | 10, 42, 32 |
| 12 | CD163L1 | CD163 molecule like 1 | 177675 | 215009 | 177575 | 39, 32, 2 |
| 12 | CD27 | CD27 molecule | 139193 | 111321 | 139192 | 41, 13, 36 |
| 12 | CD4 | CD4 molecule | 010610 | 089692 | 250510 | 39, 36, 10 |
| 12 | CD63 | CD63 molecule | 135404 | 135437 | 135414 | 35, 43, 28 |
| 12 | CD69 | CD69 molecule | 110848 | 184293 | 150045 | 5, 7, 39 |
| 12 | CD9 | CD9 molecule | 010278 | 110799 | 008323 | 30, 29, 13 |
| 12 | CDCA3 | cell division cycle associated 3 | 111665 | 111664 | 111667 | |
| 12 | CEP290 | centrosomal protein 290 | 198707 | 133641 | 139324 | 11, 12, 31 |
| 12 | CEP83 | centrosomal protein 83 | 173588 | 136040 | 057704 | |
| 12 | CERS5 | ceramide synthase 5 | 139624 | 178449 | 050405 | 18, 5, 25 |
| 12 | CCT2 | chaperonin containing TCP1 subunit 2 | 166226 | 166225 | 198812 | 43, 4, 38 |
| 12 | CHFR | checkpoint with forkhead and ring finger domains | 072609 | 283396 | 196458 | 39, 42, 38 |
| 12 | CMKLR1 | chemerin chemokine-like receptor 1 | 174600 | 075035 | 198855 | 1, 40, 44 |
| 12 | CHPT1 | choline phosphotransferase 1 | 111666 | 196091 | 139351 | |
| 12 | CBX5 | chromobox 5 | 094916 | 123415 | 135486 | 22, 12, 26 |
| 12 | CHD4 | chromodomain helicase DNA binding protein 4 | 111642 | 111641 | 184574 | 21, 40, 12 |
| 12 | C12orf10 | chromosome 12 open reading frame 10 | 139637 | 123349 | 094914 | 38, 21, 29 |
| 12 | C12orf29 | chromosome 12 open reading frame 29 | 133641 | 165805 | 198707 | 22, 1, 26 |
| 12 | C12orf4 | chromosome 12 open reading frame 4 | 047621 | 111241 | 111247 | 30, 42, 3 |
| 12 | C12orf40 | chromosome 12 open reading frame 40 | 180116 | 173208 | 151229 | 38, 25, |
| 12 | C12orf42 | chromosome 12 open reading frame 42 | 179088 | 139352 | 136011 | 38, 22, 39 |
| 12 | C12orf43 | chromosome 12 open reading frame 43 | 157895 | 135100 | 135114 | 43, 2, 41 |
| 12 | C12orf45 | chromosome 12 open reading frame 45 | 151131 | 136052 | 136010 | 3, 30, 41 |
| 12 | C12orf49 | chromosome 12 open reading frame 49 | 111412 | 258102 | 135119 | 6, 40, 23 |
| 12 | C12orf50 | chromosome 12 open reading frame 50 | 165805 | 182050 | 133641 | 38, 7, 33 |
| 12 | C12orf54 | chromosome 12 open reading frame 54 | 177627 | 139223 | 197376 | 38, |
| 12 | C12orf56 | chromosome 12 open reading frame 56 | 185306 | 174206 | 184575 | 4, 40, 32 |
| 12 | C12orf57 | chromosome 12 open reading frame 57 | 111678 | 111676 | 111679 | 15, 31, 18 |
| 12 | C12orf60 | chromosome 12 open reading frame 60 | 182993 | 084463 | 179256 | 29, 34, 18 |
| 12 | C12orf65 | chromosome 12 open reading frame 65 | 130921 | 051825 | 111328 | 30, 29, 39 |
| 12 | C12orf66 | chromosome 12 open reading frame 66 | 174206 | 196935 | 185306 | 9, 3, 13 |
| 12 | C12orf71 | chromosome 12 open reading frame 71 | 214700 | 152944 | 211455 | 10, |
| 12 | C12orf73 | chromosome 12 open reading frame 73 | 204954 | 166598 | 139372 | 43, 11, 2 |
| 12 | C12orf74 | chromosome 12 open reading frame 74 | 214215 | 257127 | 187510 | 42, 4, 22 |
| 12 | C12orf75 | chromosome 12 open reading frame 75 | 235162 | 136044 | 074590 | 45, 10, 6 |
| 12 | C12orf76 | chromosome 12 open reading frame 76 | 174456 | 076513 | 122970 | |
| 12 | CLLU1 | chronic lymphocytic leukemia up-regulated 1 | 257127 | 205057 | 214215 | |
| 12 | CLLU1OS | chronic lymphocytic leukemia up-regulated 1 opposite strand | 205057 | 133639 | 257127 | |
| 12 | CELA1 | chymotrypsin like elastase family member 1 | 139610 | 110934 | 139629 | |
| 12 | CFAP54 | cilia and flagella associated protein 54 | 188596 | 059758 | 139350 | |
| 12 | CFAP73 | cilia and flagella associated protein 73 | 186710 | 111344 | 123064 | 21, 9, 3 |
| 12 | CS | citrate synthase | 062485 | 135469 | 144785 | |
| 12 | CIT | citron rho-interacting serine/threonine kinase | 122966 | 111725 | 279777 | 27, 6, 8 |
| 12 | CPSF6 | cleavage and polyadenylation specific factor 6 | 111605 | 135678 | 090382 | 3, 36, 12 |
| 12 | COPZ1 | coatomer protein complex subunit zeta 1 | 111481 | 123405 | 139572 | 9, 34, 15 |
| 12 | COQ10A | coenzyme Q10A | 135469 | 139645 | 062485 | 26, 25, 31 |
| 12 | COQ5 | coenzyme Q5, methyltransferase | 110871 | 088986 | 022840 | 40, 23, 41 |
| 12 | CCDC184 | coiled-coil domain containing 184 | 177875 | 177981 | 172640 | 8, 2, 14 |
| 12 | CCDC38 | coiled-coil domain containing 38 | 165972 | 139343 | 139344 | 38, |
| 12 | CCDC53 | Coiled-coil domain containing 53 | 120860 | 136048 | 075188 | 2, 29, 22 |
| 12 | CCDC59 | coiled-coil domain containing 59 | 133773 | 139220 | 127720 | 35, 7, 22 |
| 12 | CCDC60 | coiled-coil domain containing 60 | 183273 | 152137 | 224982 | 42, 14, 33 |
| 12 | CCDC62 | coiled-coil domain containing 62 | 130783 | 139726 | 130787 | 38, 31, 8 |
| 12 | CCDC63 | coiled-coil domain containing 63 | 173093 | 186298 | 111245 | 38, 10, 8 |
| 12 | CCDC65 | coiled-coil domain containing 65 | 139537 | 272822 | 134285 | 30, 38, 3 |
| 12 | CCDC77 | coiled-coil domain containing 77 | 120647 | 073614 | 139044 | 12, 34, 13 |
| 12 | CCDC91 | coiled-coil domain containing 91 | 123106 | 087494 | 064763 | 10, 30, 17 |
| 12 | CCDC92 | coiled-coil domain containing 92 | 119242 | 197653 | 250091 | |
| 12 | CCER1 | coiled-coil glutamate rich protein 1 | 197651 | 070961 | 083782 | 38, |
| 12 | COL2A1 | collagen type II alpha 1 chain | 139219 | 134291 | 079387 | 11, 10, 6 |
| 12 | C1QL4 | complement C1q like 4 | 186897 | 135451 | 178401 | 37, 27, 44 |
| 12 | C1R | complement C1r | 159403 | 182326 | 139178 | 1, 6, 30 |
| 12 | C1RL | complement C1r subcomponent like | 139178 | 159403 | 139194 | 18, 43, 4 |
| 12 | C1S | complement C1s | 182326 | 111684 | 159403 | 36, 42, 10 |
| 12 | C3AR1 | complement C3a receptor 1 | 171860 | 065970 | 089818 | 10, 5, 32 |
| 12 | CNTN1 | contactin 1 | 018236 | 188906 | 165966 | 11, 26, 31 |
| 12 | COPS7A | COP9 signalosome subunit 7A | 111652 | 139200 | 089693 | 35, 34, 43 |
| 12 | CPNE8 | copine 8 | 139117 | 175548 | 139116 | 43, 29, 18 |
| 12 | CORO1C | coronin 1C | 110880 | 110876 | 084112 | 30, 45, 11 |
| 12 | COX14 | COX14, cytochrome c oxidase assembly factor | 178449 | 167588 | 139624 | 29, 31, 2 |
| 12 | CRY1 | cryptochrome circadian deck 1 | 008405 | 120832 | 151136 | 29, 38, 39 |
| 12 | CTDSP2 | CTD small phosphatase 2 | 175215 | 135407 | 166896 | 25, 22, 23 |
| 12 | CLEC1A | C-type lectin domain family 1 member A | 150048 | 197992 | 172243 | 32, 39, 11 |
| 12 | CLEC1B | C-type lectin domain family 1 member B | 165682 | 172322 | 256660 | 19, 20, 38 |
| 12 | CLEC12A | C-type lectin domain family 12 member A | 172322 | 188393 | 165682 | 7, 5, 19 |
| 12 | CLEC12B | C-type lectin domain family 12 member B | 256660 | 165682 | 197992 | 38, 20, 35 |
| 12 | CLEC2A | C-type lectin domain family 2 member A | 188393 | 256797 | 172322 | |
| 12 | CLEC2B | C-type lectin domain family 2 member B | 110852 | 150045 | 256797 | 7, 4, 26 |
| 12 | CLEC2D | C-type lectin domain family 2 member D | 069493 | 111796 | 184293 | 5, 39, 30 |
| 12 | CLEC4A | C-type lectin domain family 4 member A | 111729 | 089818 | 196946 | 30, 7, 36 |
| 12 | CLEC4C | C-type lectin domain family 4 member C | 198178 | 187569 | 205857 | 30, 5, 38 |
| 12 | CLEC4D | C-type lectin domain family 4 member D | 166527 | 205846 | 166523 | 7, 5, 36 |
| 12 | CLEC4E | C-type lectin domain family 4 member E | 166523 | 166527 | 111732 | 5, 36, 17 |
| 12 | CLEC6A | C-type lectin domain family 6 member A | 205846 | 171847 | 166527 | 20, |
| 12 | CLEC7A | C-type lectin domain family 7 member A | 172243 | 150048 | 173391 | 5, 36, 18 |
| 12 | CLEC9A | C-type lectin domain family 9 member A | 197992 | 256660 | 150048 | 32, 38, 17 |
| 12 | CLECL1 | C-type lectin like 1 | 184293 | 069493 | 110848 | 39, 22, 5 |
| 12 | CAND1 | cullin associated and neddylation dissociated 1 | 111530 | 155974 | 127334 | 22, 42, 24 |
| 12 | CUX2 | cut like homeobox 2 | 111249 | 111245 | 198324 | 11, 14, 33 |
| 12 | CCND2 | cyclin D2 | 118971 | 111224 | 078237 | 40, 11, 12 |
| 12 | CDK17 | cyclin dependent kinase 17 | 059758 | 111145 | 188596 | 26, 42, 22 |
| 12 | CDK2 | cyclin dependent kinase 2 | 123374 | 185664 | 111540 | 39, 35, 9 |
| 12 | CDK2AP1 | cyclin dependent kinase 2 associated protein 1 | 111328 | 130921 | 139697 | 45, 24, 31 |
| 12 | CDK4 | cyclin dependent kinase 4 | 135446 | 135452 | 139266 | 25, 9, 27 |
| 12 | CDKN1B | cyclin dependent kinase inhibitor 1B | 111276 | 183150 | 178878 | 11, 39, 13 |
| 12 | CCNT1 | cyclin T1 | 129315 | 139620 | 257987 | 39, 12, 31 |
| 12 | CSRP2 | cysteine and glycine rich protein 2 | 175183 | 186908 | 165891 | 43, 45, 15 |
| 12 | CSRNP2 | cysteine and serine rich nuclear protein 2 | 110925 | 050426 | 135457 | 38, 26, 31 |
| 12 | CSAD | cysteine sulfinic acid decarboxylase | 139631 | 167780 | 139651 | 26, 11, 1 |
| 12 | CMAS | cytidine monophosphate N-acetylneuraminic acid synthetase | 111726 | 069431 | 111728 | 11, 31, 3 |
| 12 | COX6A1 | cytochrome c oxidase subunit 6A1 | 111775 | 135097 | 111780 | 29, 37, 7 |
| 12 | CYP27B1 | cytochrome P450 family 27 subfamily B member 1 | 111012 | 139266 | 037897 | 17, 14, 4 |
| 12 | CKAP4 | cytoskeleton associated protein 4 | 136026 | 074590 | 166046 | 35, 19, 43 |
| 12 | DAO | D-amine acid oxidase | 110887 | 084112 | 166111 | 17, 19, 14 |
| 12 | DAZAP2 | DAZ associated protein 2 | 183283 | 184271 | 170545 | 30, 41, 36 |
| 12 | DDX11 | DEAD/H-box helicase 11 | 013573 | 110900 | 177359 | |
| 12 | DDX23 | DEAD-box helicase 23 | 174243 | 167535 | 172602 | 39, 24, 42 |
| 12 | DDX47 | DEAD-box helicase 47 | 213782 | 178878 | 013588 | 13, 43, 30 |
| 12 | DDX51 | DEAD-box helicase 51 | 185163 | 185684 | 184967 | 14, 21, 1 |
| 12 | DDX54 | DEAD-box helicase 54 | 123064 | 186710 | 139405 | 3, 21, 22 |
| 12 | DDX55 | DEAD-box helicase 55 | 111364 | 086598 | 111361 | 26, 6, 1 |
| 12 | DHX37 | DEAH-box helicase 37 | 150990 | 150991 | 184992 | 21, 27, 3 |
| 12 | DCP1B | decapping mRNAIB | 151065 | 166159 | 151067 | 38, 37, 31 |
| 12 | DCN | decorin | 011465 | 139329 | 133639 | 44, 38, |
| 12 | DTX1 | deltex E3 ubiquitin ligase 1 | 135144 | 111335 | 111344 | 21, 22, 41 |
| 12 | DTX3 | deltex E3 ubiquitin ligase 3 | 178498 | 166908 | 240771 | 28, 40, 17 |
| 12 | DDN | dendrin | 181418 | 125084 | 181929 | 9, 3, 22 |
| 12 | DENND5B | DENN domain containing 5B | 170456 | 139146 | 139160 | 5, 22, 41 |
| 12 | DENR | density regulated re-initiation and release factor | 139726 | 196917 | 130783 | 4, 12, 1 |
| 12 | DERA | deoxyribose-phosphate aldolase | 023697 | 023734 | 188991 | 4, 29, 6 |
| 12 | DEPDC4 | DEP domain containing 4 | 166153 | 075089 | 136021 | |
| 12 | DCD | dermcidin | 161634 | 135413 | 172551 | 23, |
| 12 | DHH | desert hedgehog | 139549 | 167550 | 139636 | 38, 13, 9 |
| 12 | DBX2 | developing brain homeobox 2 | 185610 | 184613 | 177119 | 6, 17, 8 |
| 12 | DPPA3 | developmental pluripotency associated 3 | 187569 | 184344 | 198178 | |
| 12 | DIABLO | diablo IAP-binding mitochondrial protein | 184047 | 176383 | 256861 | 26, 9, 37 |
| 12 | DGKA | diacylglycerol kinase alpha | 065357 | 170473 | 185664 | 39, 24, 36 |
| 12 | DIP2B | disco interacting protein 2 homolog B | 066084 | 161813 | 123268 | 11, 35, 40 |
| 12 | DDIT3 | DNA damage inducible transcript 3 | 175197 | 166986 | 166987 | 37, 25, 31 |
| 12 | DRAM1 | DNA damage regulated autophagy modulator 1 | 136048 | 111670 | 120860 | 34, 9, 40 |
| 12 | HELB | DNA helicase B | 127311 | 090376 | 155974 | 25, 45, 39 |
| 12 | POLE | DNA polymerase epsilon, catalytic subunit | 177084 | 187848 | 176894 | 29, 21, 18 |
| 12 | DNAJC14 | DnaJ heat shock protein family (Hsp40) member C14 | 135392 | 123353 | 123342 | 24, 39, 25 |
| 12 | DNAJC22 | DnaJ heat shock protein family (Hsp40) member C22 | 178401 | 186897 | 123352 | |
| 12 | DPY19L2 | dpy-19 like 2 | 177990 | 166148 | 118600 | |
| 12 | DUSP16 | dual specificity phosphatase 16 | 111266 | 165714 | 111269 | 34, 2, 39 |
| 12 | DUSP6 | dual specificity phosphatase 6 | 139318 | 049130 | 139323 | |
| 12 | DYRK2 | dual specificity tyrosine phosphorylation regulated kinase 2 | 127334 | 111530 | 111537 | 37, 24, 22 |
| 12 | DYRK4 | dual specificity tyrosine phosphorylation regulated kinase 4 | 010219 | 111247 | 272921 | 43, 38, 18 |
| 12 | DCTN2 | dynactin subunit 2 | 175203 | 166987 | 155980 | 43, 2, 9 |
| 12 | DNM1L | dynamin 1 like | 087470 | 139132 | 139131 | 24, 42, 8 |
| 12 | DNAH10 | dynein axonemal heavy chain 10 | 197653 | 185344 | 119242 | 31, 5, 30 |
| 12 | DNAH10OS | dynein axonemal heavy chain 10 opposite strand | 250091 | 119242 | 179195 | 42, 22, 5 |
| 12 | DYNLL1 | dynein light chain LC8-type 1 | 088986 | 111786 | 110871 | 45, 43, 3 |
| 12 | EP400 | E1A binding protein p400 | 183495 | 177192 | 185684 | 1, 29, 22 |
| 12 | E2F7 | E2F transcription factor 7 | 165891 | 175183 | 067798 | 9, 7, 12 |
| 12 | EEA1 | early endosome antigen 1 | 102189 | 187510 | 173598 | 26, 29, 25 |
| 12 | ETFBKMT | electron transfer flavoprotein beta subunit lysine | 139160 | 170456 | 151743 | |
| | | methyltransferase | | | | |
| 12 | ETFRF1 | electron transfer flavoprotein regulatory factor 1 | 205707 | 118307 | 133703 | |
| 12 | ELK3 | ELK3, ETS transcription factor | 111145 | 111144 | 059758 | 45, 42, 43 |
| 12 | ERC1 | ELKS/RAB6-interacting/CAST family member 1 | 082805 | 002016 | 111186 | 18, 37, 23 |
| 12 | EMG1 | EMG1, N1-specific pseudouridine methyltransferase | 126749 | 215021 | 268439 | 13, 30, 3 |
| 12 | EMG1 | EMG1, N1-specific pseudouridine methyltransferase | 268439 | 126749 | 111684 | 13, 30, 3 |
| 12 | ENDOU | endonuclease, poly(U) specific | 111405 | 005175 | 079337 | 32, 13, 24 |
| 12 | ERP27 | endoplasmic reticulum protein 27 | 139055 | 111341 | 111348 | 29, 3, 7 |
| 12 | ERP29 | endoplasmic reticulum protein 29 | 089248 | 198270 | 111300 | 5, 9, 30 |
| 12 | ENO2 | enolase 2 | 111674 | 010626 | 111676 | 8, 14, 26 |
| 12 | EID3 | EP300 interacting inhibitor of differentiation 3 | 255150 | 198431 | 171310 | 38, 18, 22 |
| 12 | EP400NL | EP400 N-terminal like | 185684 | 183495 | 185163 | 22, 38, 29 |
| 12 | EPS8 | epidermal growth factor receptor pathway substrate 8 | 151491 | 151490 | 023734 | 42, 24, 1 |
| 12 | EPYC | epiphycan | 083782 | 197651 | 139330 | 11, 32, 12 |
| 12 | EMP1 | epithelial membrane protein 1 | 134531 | 111305 | 273079 | 13, 45, 24 |
| 12 | ERBB3 | erb-b2 receptor tyrosine kinase 3 | 065361 | 197728 | 257411 | 42, 24, 11 |
| 12 | ERGIC2 | ERGIC and golgi 2 | 087502 | 064763 | 187950 | 13, 29, 30 |
| 12 | ETNK1 | ethanolamine kinase 1 | 139163 | 111731 | 134532 | 18, 11, 16 |
| 12 | ETV6 | ETS variant 6 | 139083 | 121335 | 121380 | 11, 22, 23 |
| 12 | EIF2B1 | eukaryotic translation initiation factor 2 B subunit alpha | 111361 | 111364 | 111358 | 24, 37, 2 |
| 12 | EIF4B | eukaryotic translation initiation factor 4B | 063046 | 111057 | 111077 | 42, 39, 18 |
| 12 | XPOT | exportin for tRNA | 184575 | 185306 | 183735 | 9, 6, 24 |
| 12 | ESYT1 | extended synaptotagmin 1 | 139641 | 135482 | 196465 | 43, 1, 24 |
| 12 | ESPL1 | extra spindle pole bodies like 1, separase | 135476 | 182544 | 123349 | 41, 12, 22 |
| 12 | FAM109A | family with sequence similarity 109 member A | 198324 | 111249 | 111252 | 33, 16, 14 |
| 12 | FAM186A | family with sequence similarity 186 member A | 185958 | 050405 | 161813 | 10, 22, 38 |
| 12 | FAM186B | family with sequence similarity 186 member B | 135436 | 110844 | 161791 | 38, 22, 8 |
| 12 | FAM19A2 | family with sequence similarity 19 member A2, C-C motif | 198673 | 118596 | 135655 | |
| | | chemokine like | | | | |
| 12 | FAM216A | family with sequence similarity 216 member A | 204856 | 111231 | 111237 | 2, 24, 5 |
| 12 | FAM222A | family with sequence similarity 222 member A | 139438 | 110921 | 111199 | 11, 14, 2 |
| 12 | FAM234B | family with sequence similarity 234 member B | 084444 | 013583 | 111305 | 29, 23, 8 |
| 12 | FAM60A | family with sequence similarity 60 member A | 139146 | 177359 | 170456 | 39, 27, 38 |
| 12 | FAM71C | family with sequence similarity 71 member C | 180219 | 185046 | 111647 | 38, 8, |
| 12 | FAM90A1 | family with sequence similarity 90 member A1 | 171847 | 196946 | 205846 | 38, 20, 28 |
| 12 | FAIM2 | Fas apoptotic inhibitory molecule 2 | 135472 | 186666 | 167580 | 8, 26, 14 |
| 12 | FAR2 | fatty acyl-CpA reductase 2 | 064763 | 123106 | 087502 | |
| 12 | FBXL14 | F-box and leucine rich repeat protein 14 | 171823 | 111186 | 006831 | 1, 26, 21 |
| 12 | FBXW8 | F-box and WD repeat domain containing 8 | 174989 | 135116 | 088992 | 4, 22, 31 |
| 12 | FBXO21 | F-box protein 21 | 135108 | 088992 | 089250 | 38, 26, 4 |
| 12 | FGFR1OP2 | FGFR1 oncogene partner 2 | 111790 | 064102 | 064115 | 26, 31, 5 |
| 12 | FGF23 | fibroblast growth factor 23 | 118972 | 078237 | 111241 | 12, 38, |
| 12 | FGF6 | fibroblast growth factor 6 | 111241 | 118972 | 047621 | 25, 38, |
| 12 | FRS2 | fibroblast growth factor receptor substrate 2 | 166225 | 127337 | 166226 | 24, 11, 29 |
| 12 | FBRSL1 | fibrosin like 1 | 112787 | 182870 | 204583 | 12, |
| 12 | FICD | FIC domain containing | 198855 | 174600 | 075856 | 2, 1, 13 |
| 12 | FKBP11 | FK506 binding protein 11 | 134285 | 139537 | 255863 | 35, 24, 28 |
| 12 | FKBP4 | FK506 binding protein 4 | 004478 | 151067 | 111203 | |
| 12 | FOXJ2 | forkhead box J2 | 065970 | 059804 | 171860 | 27, 43, 2 |
| 12 | FOXM1 | forkhead box M1 | 111206 | 283297 | 171792 | 21, 18, 34 |
| 12 | FOXN4 | forkhead box N4 | 139445 | 076555 | 174527 | 38, 44, 14 |
| 12 | FMNL3 | formin like 3 | 161791 | 135436 | 139644 | 22, |
| 12 | FZD10 | frizzled class receptor 10 | 111432 | 151952 | 125207 | 9, 27, 32 |
| 12 | FGD4 | FYVE, RhoGEF and PH domain containing 4 | 139132 | 151746 | 087470 | 29, 38, 1 |
| 12 | FGD6 | FYVE, RhoGEF and PH domain containing 6 | 180263 | 120798 | 028203 | 22, 26, 13 |
| 12 | GNB3 | G protein subunit beta 3 | 111664 | 110811 | 111665 | 14, 31, 25 |
| 12 | GPR162 | G protein-coupled receptor 162 | 250510 | 010610 | 110811 | |
| 12 | GPR182 | G protein-coupled receptor 182 | 166856 | 139547 | 166860 | |
| 12 | GPR19 | G protein-coupled receptor 19 | 183150 | 111269 | 111276 | 38, 12, 32 |
| 12 | GPR84 | G protein-coupled receptor 84 | 139572 | 111481 | 161642 | 20, 5, 7 |
| 12 | GPRC5A | G protein-coupled receptor class C group 5 member A | 013588 | 213782 | 111291 | 13, 16, 1 |
| 12 | GPRC5D | G protein-coupled receptor class C group 5 member D | 111291 | 013588 | 013583 | 1, 7, 33 |
| 12 | GABARAPL1 | GABA type A receptor associated protein like 1 | 139112 | 165685 | 134539 | 15, 8, 32 |
| 12 | GTSF1 | gametocyte specific factor 1 | 170627 | 161638 | 123338 | 38, 7, 34 |
| 12 | GCN1 | GCN1, elF2 alpha kinase activator homolog | 089154 | 111737 | 089157 | 31, 18, 35 |
| 12 | GRASP | general receptor for phosphoinositides 1 associated | 161835 | 135503 | 123358 | 26, 1, 36 |
| | | scaffold protein | | | | |
| 12 | GTF2H3 | general transcription factor IIH subunit 3 | 111358 | 111361 | 168778 | 45, 4, 5 |
| 12 | GSG1 | germ cell associated 1 | 111305 | 084444 | 134531 | 14, 38, 8 |
| 12 | GIT2 | GIT ArfGAP2 | 139436 | 139437 | 076513 | 39, 22, 41 |
| 12 | GLI1 | GLI family zinc finger 1 | 111087 | 139269 | 123329 | 25, 21, 12 |
| 12 | GLIPR1 | GLI pathogenesis related 1 | 139278 | 180481 | 111615 | 45, 6, 44 |
| 12 | GLIPR1L1 | GLI pathogenesis related 1 like 1 | 173401 | 180881 | 180481 | 38, 10, 23 |
| 12 | GLIPR1L2 | GLI pathogenesis related 1 like 2 | 180481 | 173401 | 139278 | 10, 38, 24 |
| 12 | GNS | glucosamine (N-acetyl)-6-sulfatase | 135677 | 153179 | 111490 | 42, 24, 2 |
| 12 | GXYLT1 | glucoside xylosyltransferase 1 | 151233 | 165966 | 015153 | 31, 18, 4 |
| 12 | GRIN2B | glutamate ionotropic receptor NMDA type subunit 2B | 273079 | 134531 | 171681 | 8, 38, 16 |
| 12 | GRIP1 | glutamate receptor interacting protein 1 | 155974 | 127311 | 111530 | 29, 32, 34 |
| 12 | GLS2 | glutaminase 2 | 135423 | 176422 | 076067 | 29, 19, 8 |
| 12 | GATC | glutamyl-tRNA amidotransferase subunit C | 257218 | 170855 | 111786 | 11, 33, 29 |
| 12 | GAPDH | glyceraldehyde-3-phosphate dehydrogenase | 111640 | 010292 | 010295 | |
| 12 | GPD1 | glycerol-3-phosphate dehydrogenase 1 | 167588 | 066117 | 178449 | 1, 26, 25 |
| 12 | GYS2 | glycogen synthase 2 | 111713 | 134548 | 111716 | 19, 10, |
| 12 | GLTP | glycolipid transfer protein | 139433 | 111199 | 139437 | 29, 18, 11 |
| 12 | GLT1D1 | glycosyltransferase 1 domain containing 1 | 151948 | 139370 | 151952 | 38, 5, 27 |
| 12 | GLT8D2 | glycosyltransferase 8 domain containing 2 | 120820 | 139372 | 111727 | 1, 43, 15 |
| 12 | GOT1B | golgi transport 1B | 111711 | 004700 | 134548 | 4, 34, 22 |
| 12 | GOLGA3 | golgin A3 | 090615 | 176915 | 283396 | 13, 29, 6 |
| 12 | GPN3 | GPN-loop GTPase 3 | 111231 | 111229 | 204856 | 34, 7, 6 |
| 12 | GAS2L3 | growth arrest specific 2 like 3 | 139354 | 012504 | 151572 | 26, 35, 23 |
| 12 | GDF11 | growth differentiation factor 11 | 135414 | 135404 | 205323 | 14, 26, 43 |
| 12 | GDF3 | growth differentiation factor 3 | 184344 | 111701 | 187569 | 12, 38, 17 |
| 12 | GUCY2C | guanylate cyclase 2C | 070019 | 121316 | 197837 | 16, 37, 25 |
| 12 | H1FNT | H1 histone family member N, testis specific | 187166 | 172640 | 167528 | 38, 32, 17 |
| 12 | H2AFJ | H2A histone family member J | 246705 | 197837 | 084463 | 29, 33, 43 |
| 12 | H3F3C | H3 histone family member 3C | 188375 | 151743 | 174718 | 30, 41, 12 |
| 12 | HRK | harakiri, BCL2 interacting protein | 135116 | 135119 | 174989 | 40, 22, 32 |
| 12 | HSP90B1 | heat shock protein 90 beta family member 1 | 166598 | 111696 | 204954 | |
| 12 | HSPB8 | heat shock protein family B (small) member 8 | 152137 | 139767 | 183273 | 32, 15, 29 |
| 12 | HECTD4 | HECT domain E3 ubiquitin protein ligase 4 | 173064 | 135148 | 089009 | |
| 12 | HEBP1 | heme binding protein 1 | 013583 | 111291 | 084444 | 10, 11, 31 |
| 12 | HNRNPA1 | heterogeneous nuclear ribonucleoprotein A1 | 135486 | 094916 | 123405 | 11, 30, 12 |
| 12 | HIGD1C | HIG1 hypoxia inducible domain family member 1C | 214511 | 185432 | 110911 | 38, |
| 12 | HMGA2 | high mobility group AT-hook 2 | 149948 | 174099 | 139233 | 12, 3, 11 |
| 12 | HAL | histidine ammonia-lyase | 084110 | 139344 | 111144 | 19, 5, 15 |
| 12 | HIST4H4 | histone cluster 4 H4 | 197837 | 070019 | 246705 | 21, |
| 12 | HDAC7 | histone deacetylase 7 | 061273 | 211584 | 111424 | 39, 26, 44 |
| 12 | HNF1A | HNF1 homeobox A | 135100 | 157837 | 157895 | 3, 19, 37 |
| 12 | HOXC10 | homeobox C10 | 180818 | 123388 | 273049 | |
| 12 | HOXC11 | homeobox C11 | 123388 | 123407 | 180818 | 3, 9, 18 |
| 12 | HOXC12 | homeobox C12 | 123407 | 123364 | 123388 | |
| 12 | HOXC13 | homeobox C13 | 123364 | 012822 | 123407 | 9, 15, 3 |
| 12 | HOXC4 | homeobox C4 | 198353 | 037965 | 172789 | 17, |
| 12 | HOXC5 | homeobox C5 | 172789 | 198353 | 123415 | |
| 12 | HOXC6 | homeobox C6 | 197757 | 273049 | 180806 | 17, |
| 12 | HOXC8 | homeobox C8 | 037965 | 180806 | 198353 | 6, 38, 23 |
| 12 | HOXC9 | homeobox C9 | 180806 | 197757 | 037965 | 6, 37, 9 |
| 12 | HCFC2 | host cell factor C2 | 111727 | 120820 | 120837 | 29, 26, 38 |
| 12 | HIP1R | huntingtin interacting protein 1 related | 130787 | 130783 | 139722 | |
| 12 | HVCN1 | hydrogen voltage gated channel 1 | 122986 | 204852 | 186298 | 41, 22, 36 |
| 12 | HCAR1 | hydroxycarboxylic acid receptor 1 | 196917 | 255398 | 139726 | |
| 12 | HCAR2 | hydroxycarboxylic acid receptor 2 | 182782 | 184445 | 255398 | 36, 1, 13 |
| 12 | HCAR3 | hydroxycarboxylic acid receptor 3 | 255398 | 182782 | 196917 | 5, 36, 4 |
| 12 | HSD17B6 | hydroxysteroid 17-beta dehydrogenase 6 | 025423 | 198056 | 170426 | 19, 1, 33 |
| 12 | IKZF4 | IKAROS family zinc finger 4 | 123411 | 139531 | 197728 | 17, 32, |
| 12 | IKBIP | IKBKB interacting protein | 166130 | 075415 | 120868 | 1, 6, 40 |
| 12 | IPO8 | importin 8 | 133704 | 133687 | 110888 | 42, 38, 29 |
| 12 | INHBC | inhibin beta C subunit | 175189 | 179912 | 139269 | 19, 24, 37 |
| 12 | INHBE | inhibin beta E subunit | 139269 | 175189 | 111087 | 25, 19, 9 |
| 12 | ING4 | inhibitor of growth family member 4 | 111653 | 111644 | 126746 | 41, 31, 26 |
| 12 | ITPR2 | inositol 1,4,5-trisphosphate receptor type 2 | 123104 | 123096 | 064102 | 22, 2, 39 |
| 12 | IGF1 | insulin like growth factor 1 | 017427 | 183395 | 171759 | 11, 33, 27 |
| 12 | IGFBP6 | insulin like growth factor binding protein 6 | 167779 | 167778 | 167780 | 1, 26, 34 |
| 12 | ITFG2 | integrin alpha FG-GAP repeat containing 2 | 111203 | 004478 | 053702 | |
| 12 | ITGA5 | integrin subunit alpha 5 | 161638 | 161642 | 170627 | |
| 12 | ITGA7 | integrin subunit alpha 7 | 135424 | 170439 | 258311 | 26, 35, 25 |
| 12 | ITGB7 | integrin subunit beta 7 | 139626 | 139651 | 172819 | 36, 39, 5 |
| 12 | IFNG | interferon gamma | 111537 | 127334 | 111536 | 5, 22, 17 |
| 12 | IRAK3 | interleukin 1 receptor associated kinase 3 | 090376 | 155957 | 127311 | 11, 5, 7 |
| 12 | IRAK4 | interleukin 1 receptor associated kinase 4 | 198001 | 129317 | 151239 | 24, 45, 13 |
| 12 | IL22 | interleukin 22 | 127318 | 111536 | 111554 | 5, |
| 12 | IL23A | interleukin 23 subunit alpha | 110944 | 135473 | 170581 | 20, 28, 22 |
| 12 | IL26 | interleukin 26 | 111536 | 111537 | 127318 | 6, |
| 12 | IL31 | interleukin 31 | 204671 | 158113 | 176383 | |
| 12 | IFFO1 | intermediate filament family orphan 1 | 010295 | 111640 | 111641 | 31, 41, 21 |
| 12 | IFT81 | intraflagellar transport 81 | 122970 | 174456 | 174437 | 13, 1, 19 |
| 12 | IQSEC3 | IQ motif and Sec7 domain 3 | 120645 | 111291 | 111181 | 1, 18, 7 |
| 12 | IQCD | IQ motif containing D | 166578 | 139405 | 186815 | 21, 38, 10 |
| 12 | ISCU | iron-sulfur cluster assembly enzyme | 136003 | 075856 | 183160 | 18, 11, 29 |
| 12 | IAPP | islet amyloid polypeptide | 121351 | 084453 | 121350 | 28, 10, 38 |
| 12 | KANSL2 | KAT8 regulatory NSL complex subunit 2 | 139620 | 167531 | 129315 | 34, 21, 25 |
| 12 | KLHL42 | kelch like family member 42 | 087448 | 205693 | 087494 | 11, 26, 22 |
| 12 | KRT1 | keratin 1 | 167768 | 172867 | 189182 | 35, 15, 39 |
| 12 | KRT18 | keratin 18 | 111057 | 170421 | 063046 | 13, 16, 28 |
| 12 | KRT2 | keratin 2 | 172867 | 186049 | 167768 | 35, 5, |
| 12 | KRT3 | keratin 3 | 186442 | 185069 | 170477 | 14, 18, 39 |
| 12 | KRT4 | keratin 4 | 170477 | 186442 | 185640 | 13, 24, 18 |
| 12 | KRT5 | keratin 5 | 186081 | 205420 | 139648 | 18, 13, 24 |
| 12 | KRT6A | keratin 6A | 205420 | 170465 | 186081 | 30, 18, 24 |
| 12 | KRT6B | keratin 6B | 185479 | 170454 | 170465 | 18, 13, 24 |
| 12 | KRT6C | keratin 6C | 170465 | 185479 | 205420 | 13, 24, 18 |
| 12 | KRT7 | keratin 7 | 135480 | 167767 | 170442 | |
| 12 | KRT71 | keratin 71 | 139648 | 186081 | 170484 | 15, 39, 35 |
| 12 | KRT72 | keratin 72 | 170486 | 170484 | 186049 | 38, 5, |
| 12 | KRT73 | keratin 73 | 186049 | 170486 | 172867 | 15, |
| 12 | KRT74 | keratin 74 | 170484 | 139648 | 170486 | 15, 38, |
| 12 | KRT75 | keratin 75 | 170454 | 161850 | 185479 | 13, 15, 24 |
| 12 | KRT76 | keratin 76 | 185069 | 189182 | 186442 | 40, 24, |
| 12 | KRT77 | keratin 77 | 189182 | 167768 | 185069 | 35, 15, 38 |
| 12 | KRT78 | keratin 78 | 170423 | 185640 | 170421 | 13, 24, 18 |
| 12 | KRT79 | keratin 79 | 185640 | 170477 | 170423 | 24, 35, 43 |
| 12 | KRT8 | keratin 8 | 170421 | 170423 | 111057 | 32, 23, 37 |
| 12 | KRT80 | keratin 80 | 167767 | 123395 | 135480 | 9, 23, 28 |
| 12 | KRT81 | keratin 81 | 205426 | 170442 | 170523 | 6, 23, 10 |
| 12 | KRT82 | keratin 82 | 161850 | 161849 | 170454 | 15, |
| 12 | KRT83 | keratin 83 | 170523 | 205426 | 135443 | 23, 28, 20 |
| 12 | KRT84 | keratin 84 | 161849 | 135443 | 161850 | 24, 28, |
| 12 | KRT85 | keratin 85 | 135443 | 170523 | 161849 | 15, 39, 44 |
| 12 | KRT86 | keratin 86 | 170442 | 135480 | 205426 | 6, 28, 20 |
| 12 | KERA | keratocan | 139330 | 083782 | 139329 | 14, 42, 32 |
| 12 | KIAA1033 | KIAA1033 | 136051 | 136010 | 136044 | 22, 12, 35 |
| 12 | KIAA1551 | KIAA1551 | 174718 | 188375 | 151746 | 22, 44, 24 |
| 12 | KLRB1 | killer cell lectin like receptor B1 | 111796 | 126838 | 069493 | 15, 30, 20 |
| 12 | KLRC1 | killer cell lectin like receptor C1 | 134545 | 205809 | 180574 | 5, 32, 44 |
| 12 | KLRC2 | killer cell lectin like receptor C2 | 205809 | 255641 | 134545 | 9, 10, 19 |
| 12 | KLRC3 | killer cell lectin like receptor C3 | 205810 | 183542 | 205809 | 10, 8, 17 |
| 12 | KLRC4 | killer cell lectin like receptor C4 | 183542 | 213809 | 255641 | 42, 36, 22 |
| 12 | KLRD1 | killer cell lectin like receptor D1 | 134539 | 139112 | 213809 | |
| 12 | KLRF1 | killer cell lectin like receptor F1 | 150045 | 110848 | 110852 | 4, 27, 10 |
| 12 | KLRF2 | killer cell lectin like receptor F2 | 256797 | 110852 | 188393 | |
| 12 | KLRG1 | killer cell lectin like receptor G1 | 139187 | 003056 | 175899 | 43, 36, 22 |
| 12 | KLRK1 | killer cell lectin like receptor K1 | 213809 | 134539 | 183542 | 36, 9, 21 |
| 12 | KSR2 | kinase suppressor of ras 2 | 171435 | 089250 | 111445 | 31, 25, 20 |
| 12 | KIF21A | kinesin family member 21A | 139116 | 139117 | 173208 | 26, 2, 9 |
| 12 | KIF5A | kinesin family member 5A | 155980 | 175203 | 166908 | 8, 26, 14 |
| 12 | KNTC1 | kinetochore associated 1 | 184445 | 111011 | 182782 | 30, 19, 10 |
| 12 | KITLG | KIT ligand | 049130 | 139324 | 139318 | 42, 25, 17 |
| 12 | KLRC4-KLRK1 | KLRC4-KLRK1 readthrough | 255819 | 134539 | 183542 | |
| 12 | KRAS | KRAS proto-oncogene, GTPase | 133703 | 205707 | 152936 | 40, 22, 27 |
| 12 | KRR1 | KRR1, small subunit processome component homolog | 111615 | 139278 | 139289 | 4, 34, 22 |
| 12 | LARP4 | La ribonucleoprotein domain family member 4 | 161813 | 185958 | 066084 | 22, 23, 26 |
| 12 | LACRT | lacritin | 135413 | 135447 | 161634 | 14, 23, |
| 12 | LALBA | lactalbumin alpha | 167531 | 197376 | 139620 | |
| 12 | LDHB | lactate dehydrogenase B | 111716 | 111713 | 121361 | 4, 41, 21 |
| 12 | LMNTD1 | lamin tail domain containing 1 | 152936 | 133703 | 123094 | |
| 12 | LRP1 | LDL receptor related protein 1 | 123384 | 166888 | 182379 | 26, 1, 6 |
| 12 | LRP6 | LDL receptor related protein 6 | 070018 | 121380 | 111261 | 29, 11, 42 |
| 12 | LEMD3 | LEM domain containing 3 | 174106 | 156076 | 174099 | 4, 22, 42 |
| 12 | LETMD1 | LETM1 domain containing 1 | 050426 | 110911 | 110925 | 9, 39, 37 |
| 12 | LRCOL1 | leucine rich colipase like 1 | 204583 | 112787 | 187848 | 39, 32, 38 |
| 12 | LRRC10 | leucine rich repeat containing 10 | 198812 | 166226 | 127325 | 15, 25, 20 |
| 12 | LRRC23 | leucine rich repeat containing 23 | 010626 | 111671 | 111674 | 41, 34, 20 |
| 12 | LRRC43 | leucine rich repeat containing 43 | 158113 | 175727 | 204671 | 38, 3, 17 |
| 12 | LGR5 | leucine rich repeat containing G protein-coupled receptor 5 | 139292 | 127324 | 133858 | 44, 11, 32 |
| 12 | LRRK2 | leucine rich repeat kinase 2 | 188906 | 151229 | 018236 | 40, 22, 7 |
| 12 | LRIG3 | leucine rich repeats and immunoglobulin like domains 3 | 139263 | 166896 | 118596 | 42, 24, 18 |
| 12 | LRRIQ1 | leucine rich repeats and IQ motif containing 1 | 133640 | 231738 | 180318 | 38, 20, 42 |
| 12 | LRTM2 | leucine rich repeats and transmembrane domains 2 | 166159 | 151062 | 151065 | 8, 17, 14 |
| 12 | LTA4H | leukotriene A4 hydrolase | 111144 | 084110 | 111145 | 36, 39, 10 |
| 12 | LIMA1 | LIM domain and actin binding 1 | 050405 | 139624 | 185958 | 13, 45, 24 |
| 12 | LMO3 | LIM domain only 3 | 048540 | 008394 | 111404 | 8, 40, 42 |
| 12 | LHX5 | LIM homeobox 5 | 089116 | 139410 | 122965 | |
| 12 | LMBR1L | limb development membrane protein 1 like | 139636 | 139549 | 123416 | 39, 36, 45 |
| 12 | LIN7A | lin-7 homolog A, crumbs cell polarity complex component | 111052 | 111049 | 111058 | 10, 38, 30 |
| 12 | LLPH | LLP homolog, long-term synaptic facilitation | 139233 | 149948 | 228144 | 4, 7, 30 |
| 12 | LINC00935 | Long intergenic non-protein coding RNA 935 | 257987 | 129315 | 174233 | |
| 12 | LUM | lumican | 139329 | 139330 | 011465 | 43, 10, 32 |
| 12 | LAG3 | lymphocyte activating 3 | 089692 | 159335 | 010610 | 10, 36, 37 |
| 12 | LRMP | lymphoid restricted membrane protein | 118308 | 060982 | 118307 | 39, 22, 21 |
| 12 | LTBR | lymphotoxin beta receptor | 111321 | 111319 | 139193 | 3, 37, 4 |
| 12 | KDM2B | lysine demethylase 2B | 089094 | 170633 | 276045 | 11, 9, 21 |
| 12 | KDM5A | lysine demethylase 5A | 073614 | 010379 | 120647 | 45, 26, 36 |
| 12 | KMT2D | lysine methyltransferase 2D | 167548 | 181929 | 167550 | 5, 18, 4 |
| 12 | KMT5A | lysine methyltransferase 5A | 183955 | 139697 | 150977 | 18, 6, 21 |
| 12 | LPAR5 | lysophosphatidic acid receptor 5 | 184574 | 111642 | 111644 | 41, 36, 4 |
| 12 | LPCAT3 | lysophosphatidylcholine acyltransferase 3 | 111684 | 268439 | 182326 | 41, 39, 2 |
| 12 | LYZ | lysozyme | 090382 | 111605 | 127337 | 30, 18, 42 |
| 12 | MAGOHB | mago homolog B, exon junction complex core component | 111196 | 180574 | 060140 | 34, 7, 4 |
| 12 | MFSD5 | major facilitator superfamily domain containing 5 | 182544 | 283536 | 135476 | 9, 37, 5 |
| 12 | MIP | major intrinsic protein of lens fiber | 135517 | 111602 | 176422 | 14, 2, 23 |
| 12 | MLEC | malectin | 110917 | 157782 | 175970 | |
| 12 | M6PR | mannose-6-phosphate receptor, cation dependent | 003056 | 111752 | 139187 | 21, 38, 36 |
| 12 | MANSC1 | MANSC domain containing 1 | 111261 | 070018 | 165714 | 42, 45, 2 |
| 12 | MANSC4 | MANSC domain containing 4 | 205693 | 061794 | 087448 | 28, 20, |
| 12 | MGP | matrix Gla protein | 111341 | 111339 | 139055 | 45, 43, 11 |
| 12 | MMP17 | matrix metallopeptidase 17 | 198598 | 061936 | 177169 | 26, 35, 14 |
| 12 | MMP19 | matrix metallopeptidase 19 | 123342 | 135392 | 170473 | 32, 20, 31 |
| 12 | MDM1 | Mdm1 nuclear protein | 111554 | 127318 | 127314 | 10, 11, 38 |
| 12 | MDM2 | MDM2 proto-oncogene | 135679 | 175782 | 135678 | 40, 24, 5 |
| 12 | MED13L | mediator complex subunit 13 like | 123066 | 135111 | 258102 | 38, |
| 12 | MED21 | mediator complex subunit 21 | 152944 | 064115 | 214700 | 6, |
| 12 | MARCH9 | membrane associated ring-CH-type finger 9 | 139266 | 135446 | 111012 | 34, 18, 31 |
| 12 | MSRB3 | methionine sulfoxide reductase B3 | 174099 | 174106 | 149948 | 45, 44, 9 |
| 12 | METAP2 | methionyl aminopeptidase 2 | 111142 | 028203 | 136014 | 43, 13, 45 |
| 12 | MARS | methionyl-tRNA synthetase | 166986 | 123329 | 175197 | 25, 24, 3 |
| 12 | MBD6 | methyl-CpG binding domain protein 6 | 166987 | 175197 | 175203 | 25, 40, 18 |
| 12 | MMAB | methylmalonic aciduria (cobalamin deficiency) cbIB type | 139428 | 151148 | 110921 | 34, 41, 2 |
| 12 | METTL1 | methyltransferase like 1 | 037897 | 111012 | 123427 | 18, 25, 27 |
| 12 | METTL21B | methyltransferase like 21B | 123427 | 037897 | 257921 | 13, 24, 17 |
| 12 | METTL25 | methyltransferase like 25 | 127720 | 133773 | 179104 | 10, 45, 29 |
| 12 | METTL7A | methyltransferase like 7A | 185432 | 186452 | 214511 | 29, 1, 17 |
| 12 | METTL7B | methyltransferase like 7B | 170439 | 175398 | 135424 | 3, 40, 16 |
| 12 | MVK | mevalonate kinase | 110921 | 139428 | 139438 | 21, 9, 38 |
| 12 | MGAT4C | MGAT4 family member C | 182050 | 133636 | 165805 | |
| 12 | MFAP5 | microfibrillar associated protein 5 | 197614 | 111732 | 166532 | 45, 1, 15 |
| 12 | MGST1 | microsomal glutathione S-transferase 1 | 008394 | 188991 | 048540 | |
| 12 | MCRS1 | microspherule protein 1 | 187778 | 135519 | 110844 | 9, 5, 34 |
| 12 | MAP1LC3B2 | microtubule associated protein 1 light chain 3 beta 2 | 258102 | 123066 | 111412 | 12, |
| 12 | MRPL42 | mitochondrial ribosomal protein L42 | 198015 | 177889 | 120833 | 28, 32, |
| 12 | MRPL51 | mitochondrial ribosomal protein L51 | 111639 | 139190 | 010292 | 41, 31, 30 |
| 12 | MRPS35 | mitochondrial ribosomal protein S35 | 061794 | 174236 | 205693 | 3, 7, 4 |
| 12 | MTERF2 | mitochondrial transcription termination factor 2 | 120832 | 151135 | 008405 | 1, 43, 7 |
| 12 | MAP3K12 | mitogen-activated protein kinase kinase kinase 12 | 139625 | 197111 | 139546 | 26, 18, 31 |
| 12 | MAPKAPK5 | mitogen-activated protein kinase-activated protein kinase 5 | 089022 | 111275 | 198270 | 11, 9, 3 |
| 12 | MLXIP | MLX interacting protein | 175727 | 110987 | 158113 | |
| 12 | MON2 | MON2 homolog, regulator of endosome-to-Golgi trafficking | 061987 | 135655 | 111110 | 11, 22, 1 |
| 12 | MORN3 | MORN repeat containing 3 | 139714 | 276045 | 188735 | 43, 38, 14 |
| 12 | MPHOSPH9 | M-phase phosphoprotein 9 | 051825 | 090975 | 130921 | |
| 12 | MUCL1 | mucin like 1 | 172551 | 161634 | 135426 | 23, 10, 15 |
| 12 | MSI1 | musashi RNA binding protein 1 | 135097 | 170890 | 111775 | 14, 12, 38 |
| 12 | MYRFL | myelin regulatory factor-like | 166268 | 258052 | 111596 | 10, 17, 20 |
| 12 | MLF2 | myeloid leukemia factor 2 | 089693 | 111652 | 159335 | 26, 21, 27 |
| 12 | MYF5 | myogenic factor 5 | 111049 | 111046 | 111052 | 24, 25, 23 |
| 12 | MYF6 | myogenic factor 6 | 111046 | 139304 | 111049 | 25, 15, 19 |
| 12 | MYBPC1 | myosin binding protein C, slow type | 196091 | 166211 | 111666 | 25, 24, 26 |
| 12 | MYO1A | myosin IA | 166866 | 166863 | 166881 | 16, 3, 37 |
| 12 | MYO1H | myosin IH | 174527 | 139445 | 110906 | 39, 37, 17 |
| 12 | MYL2 | myosin light chain 2 | 111245 | 173093 | 111249 | 15, 25, 18 |
| 12 | MYL6 | myosin light chain 6 | 092841 | 196465 | 139613 | 31, 30, 28 |
| 12 | MYL6B | myosin light chain 6B | 196465 | 139641 | 092841 | 34, 25, 15 |
| 12 | NAA25 | N(alpha)-acetyltransferase 25, NatB auxiliary subunit | 111300 | 089248 | 135148 | 22, 45, 3 |
| 12 | GNPTAB | N-acetylglucosamine-1-phosphate transferase alpha | 111670 | 139351 | 136048 | 18, 39, 26 |
| | | and beta subunits | | | | |
| 12 | NDUFA12 | NADH: ubiquinone oxidoreductase subunit A12 | 184752 | 057704 | 120798 | 10, 3, |
| 12 | NDUFA9 | NADH: ubiquinone oxidoreductase subunit A9 | 139180 | 111254 | 255639 | 30, 4, 34 |
| 12 | NANOG | Nanog homeobox | 111704 | 205857 | 173262 | 5, 38, 12 |
| 12 | NANOGNB | NANOG neighbor homeobox | 205857 | 198178 | 111704 | 38, |
| 12 | NACA | nascent polypeptide-associated complex alpha subunit | 196531 | 110958 | 198056 | 18, 11, 43 |
| 12 | NCKAP1L | NCK associated protein 1 like | 123338 | 170627 | 123360 | 39, 21, 36 |
| 12 | NCKAP5L | NCK associated protein 5 like | 167566 | 139644 | 186666 | 25, 35, 12 |
| 12 | NDUFA4L2 | NDUFA4, mitochondrial complex associated like 2 | 185633 | 182199 | 185482 | 14, |
| 12 | NECAP1 | NECAP endocytosis associated 1 | 089818 | 171860 | 111729 | 31, 8, 1 |
| 12 | NTN4 | netrin 4 | 074527 | 136014 | 139343 | 45, 6, 4 |
| 12 | NELL2 | neural EGFL like 2 | 184613 | 139173 | 185610 | 8, 31, 40 |
| 12 | NEDD1 | neural precursor cell expressed, developmentally | 139350 | 188596 | 120802 | 4, 39, 35 |
| | | down-regulated 1 | | | | |
| 12 | NXPH4 | neurexophilin 4 | 182379 | 123384 | 182199 | 25, 6, 14 |
| 12 | NAV3 | neuron navigator 3 | 067798 | 165891 | 067715 | 11, 8, 36 |
| 12 | NEUROD4 | neuronal differentiation 4 | 123307 | 135426 | 170605 | 14, 8, |
| 12 | NPFF | neuropeptide FF-amide peptide precursor | 139574 | 267281 | 170653 | 28, 22, 17 |
| 12 | NTS | neurotensin | 133636 | 198774 | 182050 | 10, 37, 31 |
| 12 | NTF3 | neurotrophin 3 | 185652 | 130037 | 047617 | 32, 27, 12 |
| 12 | NAB2 | NGFI-A binding protein 2 | 166886 | 166881 | 166888 | 26, 27, 40 |
| 12 | NINJ2 | ninjurin 2 | 171840 | 139044 | 060237 | 22, 7, 5 |
| 12 | NOS1 | nitric oxide synthase 1 | 089250 | 135108 | 171435 | 25, 20, 8 |
| 12 | NCAPD2 | non-SMC condensin I complex subunit D2 | 010292 | 111639 | 111640 | 43, 22, 39 |
| 12 | NOP2 | NOP2 nucleolar protein | 111641 | 010295 | 111642 | 39, 24, 38 |
| 12 | NUAK1 | NUAK family kinase 1 | 074590 | 235162 | 136026 | 1, 18, 43 |
| 12 | NEMP1 | nuclear envelope integral membrane protein 1 | 166881 | 166866 | 166886 | 18, 11, 39 |
| 12 | NFE2 | nuclear factor, erythroid 2 | 123405 | 135486 | 111481 | 7, 5, 15 |
| 12 | NCOR2 | nuclear receptor corepressor 2 | 196498 | 274874 | 073060 | 18, 22, 35 |
| 12 | NRIP2 | nuclear receptor interacting protein 2 | 053702 | 111203 | 283297 | 18, 8, 42 |
| 12 | NR1H4 | nuclear receptor subfamily 1 group H member 4 | 012504 | 179520 | 139354 | 2, 4, 17 |
| 12 | NR2C1 | nuclear receptor subfamily 2 group C member 1 | 120798 | 184752 | 180263 | 39, 36, 11 |
| 12 | NR4A1 | nuclear receptor subfamily 4 group A member 1 | 123358 | 161835 | 123395 | |
| 12 | NFYB | nuclear transcription factor Y subunit beta | 120837 | 111727 | 198431 | 4, 29, 34 |
| 12 | NABP2 | nucleic acid binding protein 2 | 139579 | 181852 | 139540 | 9, 34, 27 |
| 12 | NOC4L | nucleolar complex associated 4 homolog | 184967 | 185163 | 182870 | 26, 41, 21 |
| 12 | NUP107 | nudeoporin 107 | 111581 | 127314 | 175782 | 11, 32, 12 |
| 12 | NUP37 | nudeoporin 37 | 075188 | 120860 | 281370 | 3, 34, 1 |
| 12 | NAP1L1 | nucleosome assembly protein 1 like 1 | 187109 | 139289 | 179941 | |
| 12 | NUDT4 | nudix hydrolase 4 | 173598 | 102189 | 177889 | 45, 42, 34 |
| 12 | OR10A7 | olfactory receptor family 10 subfamily A member 7 | 179919 | 170605 | 197706 | |
| 12 | OR10AD1 | olfactory receptor family 10 subfamily AD member 1 | 172640 | 177875 | 187166 | 25, 44, 38 |
| 12 | OR10P1 | olfactory receptor family 10 subfamily P member 1 | 175398 | 179615 | 170439 | |
| 12 | OR2AP1 | olfactory receptor family 2 subfamily AP member 1 | 179615 | 179626 | 175398 | |
| 12 | OR6C1 | olfactory receptor family 6 subfamily C member 1 | 205330 | 188324 | 205329 | |
| 12 | OR6C2 | olfactory receptor family 6 subfamily C member 2 | 179695 | 185821 | 184954 | 10, |
| 12 | OR6C3 | olfactory receptor family 6 subfamily C member 3 | 205329 | 205330 | 187857 | |
| 12 | OR6C4 | olfactory receptor family 6 subfamily C member 4 | 179626 | 205327 | 179615 | |
| 12 | OR6C6 | olfactory receptor family 6 subfamily C member 6 | 188324 | 197706 | 205330 | |
| 12 | OR6C65 | olfactory receptor family 6 subfamily C member 65 | 205328 | 187857 | 185821 | |
| 12 | OR6C68 | olfactory receptor family 6 subfamily C member 68 | 205327 | 184954 | 179626 | 10, |
| 12 | OR6C70 | olfactory receptor family 6 subfamily C member 70 | 184954 | 179695 | 205327 | |
| 12 | OR6C74 | olfactory receptor family 6 subfamily C member 74 | 197706 | 179919 | 188324 | |
| 12 | OR6C75 | olfactory receptor family 6 subfamily C member 75 | 187857 | 205329 | 205328 | |
| 12 | OR6C76 | olfactory receptor family 6 subfamily C member 76 | 185821 | 205328 | 179695 | |
| 12 | OR8S1 | olfactory receptor family 8 subfamily S member 1 | 197376 | 177627 | 167531 | |
| 12 | OR9K2 | olfactory receptor family 9 subfamily K member 2 | 170605 | 123307 | 179919 | |
| 12 | ORAI1 | ORAI calcium release-activated calcium modulator 1 | 276045 | 089094 | 139714 | 27, 3, 22 |
| 12 | ORMDL2 | ORMDL sphingolipid biosynthesis regulator 2 | 123353 | 257390 | 135392 | 11, 13, 30 |
| 12 | OS9 | OS9, endoplasmic reticulum lectin | 135506 | 135454 | 135439 | 24, 36, 45 |
| 12 | OTOGL | otogelin like | 165899 | 058272 | 139304 | 11, 45, 37 |
| 12 | OVCH1 | ovochymase 1 | 187950 | 087502 | 133687 | 25, 19, 38 |
| 12 | OLR1 | oxidized low density lipoprotein receptor 1 | 173391 | 172243 | 165685 | 13, 32, 17 |
| 12 | OSBPL8 | oxysterol binding protein like 8 | 091039 | 179941 | 186908 | 22, 7, 13 |
| 12 | PAN2 | PAN2 poly(A) specific ribonuclease subunit | 135473 | 257727 | 110944 | 40, 1, 22 |
| 12 | PTMS | parathymosin | 159335 | 089693 | 089692 | 34, 6, 14 |
| 12 | PTHLH | parathyroid hormone like hormone | 087494 | 087448 | 123106 | 18, 24, 26 |
| 12 | PARPBP | PARP1 binding protein | 185480 | 281370 | 183395 | 4, 38, 7 |
| 12 | PXN | paxillin | 089159 | 089157 | 089163 | 42, 35, 41 |
| 12 | PCED1B | PC-esterase domain containing 1B | 179715 | 139211 | 005175 | 41, 22, 5 |
| 12 | PDZRN4 | PDZ domain containing ring finger 4 | 165966 | 018236 | 151233 | 26, 11, 27 |
| 12 | PRPH | peripherin | 135406 | 167553 | 135451 | |
| 12 | PPHLN1 | periphilin 1 | 134283 | 015153 | 139168 | 6, 3, 15 |
| 12 | PEX5 | peroxisomal biogenesis factor 5 | 139197 | 139182 | 215009 | 39, 24, 38 |
| 12 | PXMP2 | peroxisomal membrane protein 2 | 176894 | 177084 | 256632 | 17, 6, 31 |
| 12 | PGAM5 | PGAM family member 5, mitochondrial serine/threonine | 247077 | 256632 | 176915 | 9, 4, 27 |
| | | protein phosphatase | | | | |
| 12 | PAH | phenylalanine hydroxylase | 171759 | 017427 | 139352 | 19, 17, 4 |
| 12 | PEBP1 | phosphatidylethanolamine binding protein 1 | 089220 | 176834 | 135090 | 2, 28, 29 |
| 12 | PITPNM2 | phosphatidylinositol transfer protein membrane associated 2 | 090975 | 182196 | 051825 | 39, 3, 14 |
| 12 | PIK3C2G | phosphatidylinositol-4-phosphate 3-kinase catalytic | 139144 | 111404 | 139151 | 10, 17, 42 |
| | | subunit type 2 gamma | | | | |
| 12 | PIP4K2C | phosphatidylinositol-5-phosphate 4-kinase type 2 gamma | 166908 | 155980 | 178498 | 38, 26, 31 |
| 12 | PDE1B | phosphodiesterase 1B | 123360 | 123338 | 135447 | 8, 1, 36 |
| 12 | PDE3A | phosphodiesterase 3A | 172572 | 139154 | 139155 | 45, 26, 11 |
| 12 | PDE6H | phosphodiesterase 6H | 139053 | 111348 | 134533 | 14, 8, |
| 12 | PFKM | phosphofructokinase, muscle | 152556 | 079387 | 177981 | 15, 24, 8 |
| 12 | PLA2G1B | phospholipase A2 group IB | 170890 | 089163 | 135097 | 28, 4, 10 |
| 12 | PLBD1 | phospholipase B domain containing 1 | 121316 | 171681 | 070019 | 24, 32, 6 |
| 12 | PLBD2 | phospholipase B domain containing 2 | 151176 | 089060 | 135094 | 26, 34, 13 |
| 12 | PLCZ1 | phospholipase C zeta 1 | 139151 | 139144 | 177938 | |
| 12 | PIANP | PILR alpha associated neural protein | 139200 | 126746 | 111652 | 31, 8, 14 |
| 12 | PIWIL1 | piwi like RNA-mediated gene silencing 1 | 125207 | 111432 | 060709 | 38, 25, 14 |
| 12 | PKP2 | plakophilin 2 | 057294 | 139131 | 110975 | 16, 15, 4 |
| 12 | PLEKHG6 | pleckstrin homology and RhoGEF domain containing G6 | 008323 | 010278 | 067182 | 3, 34, 16 |
| 12 | PLEKHG7 | pleckstrin homology and RhoGEF domain containing G7 | 187510 | 214215 | 102189 | 42, 18, 22 |
| 12 | PLEKHA5 | pleckstrin homology domain containing A5 | 052126 | 177938 | 139154 | 1, 17, 25 |
| 12 | PHLDA1 | pleckstrin homology like domain family A member 1 | 139289 | 111615 | 187109 | 6, 23, 42 |
| 12 | PLXNC1 | plexin C1 | 136040 | 169372 | 173588 | 35, 42, 22 |
| 12 | POC1B | POC1 centriolar protein B | 139323 | 139318 | 259075 | 42, 18, 16 |
| 12 | POC1B-GALNT4 | POC1B-GALNT4 readthrough | 259075 | 139323 | 070961 | |
| 12 | PARP11 | poly(ADP-ribose) polymerase family member 11 | 111224 | 130038 | 118971 | 22, 31, 15 |
| 12 | PCBP2 | poly(rC) binding protein 2 | 197111 | 205352 | 139625 | 18, 40, 10 |
| 12 | PHC1 | polyhomeotic homolog 1 | 111752 | 166535 | 003056 | 12, 38, 27 |
| 12 | GALNT4 | polypeptide N-acetylgalactosaminyltransferase 4 | 257594 | 139323 | 070961 | |
| 12 | GALNT6 | polypeptide N-acetylgalactosaminyltransferase 6 | 139629 | 139610 | 050438 | 23, 24, 45 |
| 12 | GALNT8 | polypeptide N-acetylgalactosaminyltransferase 8 | 130035 | 255639 | 151079 | 15, 8, 20 |
| 12 | GALNT9 | polypeptide N-acetylgalactosaminyltransferase 9 | 182870 | 184967 | 112787 | 8, 17, 20 |
| 12 | POP5 | POP5 homolog, ribonuclease P/MRP subunit | 167272 | 022840 | 157782 | 2, 6, 16 |
| 12 | KCNMB4 | potassium calcium-activated channel subfamily M | 135643 | 279530 | 127329 | 22, 45, 16 |
| | | regulatory beta subunit 4 | | | | |
| 12 | KCTD10 | potassium channel tetramerization domain containing 10 | 110906 | 174527 | 151148 | 42, 1, 40 |
| 12 | KCNA1 | potassium voltage-gated channel subfamily A member 1 | 111262 | 151079 | 130037 | |
| 12 | KCNA5 | potassium voltage-gated channel subfamily A member 5 | 130037 | 111262 | 185652 | 27, 28, 14 |
| 12 | KCNA6 | potassium voltage-gated channel subfamily A member 6 | 151079 | 130035 | 111262 | 15, 8, 20 |
| 12 | KCNC2 | potassium voltage-gated channel subfamily C member 2 | 166006 | 253719 | 180881 | 33, 31, 10 |
| 12 | KCNH3 | potassium voltage-gated channel subfamily H member 3 | 135519 | 123352 | 187778 | 26, 31, 20 |
| 12 | KCNJ8 | potassium voltage-gated channel subfamily J member 8 | 121361 | 111716 | 069431 | 40, 32, 36 |
| 12 | POU6F1 | POU class 6 homeobox 1 | 184271 | 135457 | 183283 | 36, 34, 8 |
| 12 | PPFIBP1 | PPFIA binding protein 1 | 110841 | 165935 | 174236 | 1, 25, 18 |
| 12 | PRDM4 | PR/SET domain 4 | 110851 | 136045 | 187855 | 29, 13, 2 |
| 12 | PFDN5 | prefoldin subunit 5 | 123349 | 135476 | 139637 | 40, 31, 41 |
| 12 | PMEL | premelanosome protein | 185664 | 065357 | 123374 | 35, 14, 44 |
| 12 | PRPF40B | pre-mRNA processing factor 40 homolog B | 110844 | 187778 | 135436 | |
| 12 | PRICKLE1 | prickle planar cell polarity protein 1 | 139174 | 139168 | 173157 | |
| 12 | PRIM1 | primase (DNA) subunit 1 | 198056 | 196531 | 025423 | 4, 41, 18 |
| 12 | PAWR | pro-apoptotic WT1 regulator | 177425 | 067715 | 058272 | 23, 18, 31 |
| 12 | PHB2 | prohibitin 2 | 215021 | 111679 | 126749 | 41, 21, 9 |
| 12 | PA2G4 | proliferation-associated 2G4 | 170515 | 257411 | 229117 | 21, 41, 43 |
| 12 | PRR13 | proline rich 13 | 205352 | 135409 | 197111 | 41, 30, 21 |
| 12 | PRR4 | proline rich 4 (lacrimal) | 111215 | 121318 | 212128 | 18, 42, 14 |
| 12 | PRB1 | proline rich protein BstNI subfamily 1 | 251655 | 230657 | 121335 | 34, 25, 10 |
| 12 | PRB2 | proline rich protein BstNI subfamily 2 | 121335 | 251655 | 139083 | 25, 20, 16 |
| 12 | PRB3 | proline rich protein BstNI subfamily 3 | 197870 | 186136 | 230657 | 18, 34, 25 |
| 12 | PRB4 | proline rich protein BstNI subfamily 4 | 230657 | 197870 | 251655 | 25, |
| 12 | PRH1 | proline rich protein HaeIII subfamily 1 | 231887 | 121381 | 121318 | 18, 34, 2 |
| 12 | PRH2 | proline rich protein HaeIII subfamily 2 | 134551 | 212128 | 212127 | 18, 25, 34 |
| 12 | P3H3 | prolyl 3-hydroxylase 3 | 110811 | 250510 | 111664 | 31, 34, 45 |
| 12 | PMCH | pro-melanin concentrating hormone | 183395 | 185480 | 017427 | 2, 8, 17 |
| 12 | PTGES3 | prostaglandin E synthase 3 | 110958 | 110955 | 196531 | 4, 13, 2 |
| 12 | PSMD9 | proteasome 26S subunit, non-ATPase 9 | 110801 | 158104 | 256950 | 11, 37, 13 |
| 12 | PRMT8 | protein arginine methyltransferase 8 | 111218 | 011105 | 130038 | 8, 39, 30 |
| 12 | PRKAB1 | protein kinase AMP-activated non-catalytic subunit beta 1 | 111725 | 224982 | 122966 | 36, 29, 16 |
| 12 | PRKAG1 | protein kinase AMP-activated non-catalytic subunit gamma 1 | 181929 | 181418 | 167548 | 38, |
| 12 | PPP1CC | protein phosphatase 1 catalytic subunit gamma | 186298 | 122986 | 173093 | 21, 4, 29 |
| 12 | PPP1R1A | protein phosphatase 1 regulatory inhibitor subunit 1A | 135447 | 123360 | 135413 | 25, 28, 26 |
| 12 | PPP1R12A | protein phosphatase 1 regulatory subunit 12A | 058272 | 177425 | 165899 | 11, 1, 45 |
| 12 | PPM1H | protein phosphatase, Mg2+/Mn2+ dependent 1H | 111110 | 061987 | 166148 | 11, 4, 18 |
| 12 | PTPN11 | protein tyrosine phosphatase, non-receptor type 11 | 179295 | 089009 | 089169 | 29, 26, 18 |
| 12 | PTPN6 | protein tyrosine phosphatase, non-receptor type 6 | 111679 | 111678 | 215021 | 36, 41, 5 |
| 12 | PTPRB | protein tyrosine phosphatase, receptor type B | 127329 | 135643 | 153233 | 43, 1, 17 |
| 12 | PTPRO | protein tyrosine phosphatase, receptor type O | 151490 | 134533 | 151491 | 8, 7, 17 |
| 12 | PTPRQ | protein tyrosine phosphatase, receptor type Q | 139304 | 165899 | 111046 | 45, 37, 38 |
| 12 | PTPRR | protein tyrosine phosphatase, receptor type R | 153233 | 127329 | 127324 | 26, 9, 4 |
| 12 | PUS1 | pseudouridylate synthase 1 | 177192 | 177169 | 183495 | 9, 1, 3 |
| 12 | PUS7L | pseudouridylate synthase 7 like | 129317 | 173157 | 198001 | 34, 1, 4 |
| 12 | PPTC7 | PTC7 protein phosphatase homolog | 196850 | 151164 | 204852 | 1, 22, 3 |
| 12 | PPFIA2 | PTPRF interacting protein alpha 2 | 139220 | 111058 | 133773 | |
| 12 | P2RX2 | purinergic receptor P2X2 | 187848 | 204583 | 177084 | 38, 17, |
| 12 | P2RX4 | purinergic receptor P2X4 | 135124 | 089041 | 110931 | 5, 29, 32 |
| 12 | P2RX7 | purinergic receptor P2X7 | 089041 | 135114 | 135124 | 36, 10, 8 |
| 12 | EIF2S3L | Putative eukaryotic translation initiation factor 2 | 180574 | 134545 | 111196 | |
| | | subunit 3-like protein | | | | |
| 12 | PWP1 | PWP1 homolog, endonuclein | 136045 | 151136 | 110851 | 41, 35, 17 |
| 12 | PYM1 | PYM homolog 1, exon junction complex associated factor | 170473 | 123342 | 065357 | 41, 29, 40 |
| 12 | PYROXD1 | pyridine nucleotide-disulphide oxidoreductase domain 1 | 121350 | 121351 | 004700 | 45, 22, 30 |
| 12 | PZP | PZP, alpha-2-macroglobulin like | 126838 | 175899 | 111796 | 42, 19, 9 |
| 12 | R3HDM2 | R3H domain containing 2 | 179912 | 258830 | 175189 | 29, 18, 40 |
| 12 | RILPL1 | Rab interacting lysosomal protein like 1 | 188026 | 184209 | 086598 | 43, 2, 15 |
| 12 | RILPL2 | Rab interacting lysosomal protein like 2 | 150977 | 183955 | 184209 | 1, 34, 36 |
| 12 | REP15 | RAB15 effector protein | 174236 | 110841 | 061794 | 23, 10, 28 |
| 12 | RAB21 | RAB21, member RAS oncogene family | 080371 | 139291 | 121749 | 31, 32, 1 |
| 12 | RAB35 | RAB35, member RAS oncogene family | 111737 | 135127 | 089154 | 26, 39, 31 |
| 12 | RAB3IP | RAB3A interacting protein | 127328 | 127325 | 258052 | 38, 17, 20 |
| 12 | RAB5B | RAB5B, member RAS oncogene family | 111540 | 123374 | 139531 | 26, 42, 24 |
| 12 | RPH3A | rabphilin 3A | 089169 | 179295 | 089127 | 8, 14, 2 |
| 12 | RACGAP1 | Rac GTPase activating protein 1 | 161800 | 086159 | 110881 | 39, 38, 9 |
| 12 | RAD51AP1 | RAD51 associated protein 1 | 111247 | 047621 | 010219 | 4, 13, 7 |
| 12 | RAD52 | RAD52 homolog, DNA repair protein | 002016 | 060237 | 082805 | 10, 39, 2 |
| 12 | RAD9B | RAD9 checkpoint clamp component B | 151164 | 111237 | 196850 | 38, 30, 9 |
| 12 | RHNO1 | RAD9-HUS1-RAD1 interacting nuclear orphan 1 | 171792 | 111206 | 078246 | 13, 9, 30 |
| 12 | RAN | RAN, member RAS oncogene family | 132341 | 111450 | 111452 | 41, 34, 21 |
| 12 | RAPGEF3 | Rap guanine nucleotide exchange factor 3 | 079337 | 111405 | 211584 | 43, 1, 40 |
| 12 | RAP1B | RAP1B, member of RAS oncogene family | 127314 | 111554 | 111581 | 13, 45, 39 |
| 12 | RASSF3 | Ras association domain family member 3 | 153179 | 183735 | 135677 | 33, 1, 40 |
| 12 | RASSF8 | Ras association domain family member 8 | 123094 | 152936 | 123095 | 38, 1, 3 |
| 12 | RASSF9 | Ras association domain family member 9 | 198774 | 180318 | 133636 | 43, 27, 42 |
| 12 | RHEBL1 | Ras homolog enriched in brain like 1 | 167550 | 167548 | 139549 | 5, 21, 15 |
| 12 | RHOF | ras homolog family member F, filopodia associated | 139725 | 188735 | 139718 | |
| 12 | RERG | RAS like estrogen regulated growth inhibitor | 134533 | 139053 | 151490 | 26, 45, 2 |
| 12 | RASAL1 | RAS protein activator like 1 | 111344 | 135144 | 186710 | 21, 41, 2 |
| 12 | RITA1 | RBPJ interacting and tubulin associated 1 | 139405 | 123064 | 166578 | |
| 12 | RECQL | RecQ like helicase | 004700 | 121350 | 111711 | 42, 45, 26 |
| 12 | RFLNA | refilin A | 178882 | 179195 | 274874 | 26, 37, 14 |
| 12 | RFX4 | regulatory factor X4 | 111783 | 013503 | 111785 | 38, 8, 12 |
| 12 | RFC5 | replication factor C subunit 5 | 111445 | 171435 | 176871 | 39, 9, 13 |
| 12 | RERGL | RERG like | 111404 | 048540 | 139144 | 26, 45, 7 |
| 12 | RARG | retinoic acid receptor gamma | 172819 | 139626 | 283536 | 24, 4, 44 |
| 12 | RBP5 | retinol binding protein 5 | 139194 | 139178 | 139182 | 36, 4, 17 |
| 12 | RDH16 | retinol dehydrogenase 16 (all-trans) | 139547 | 170426 | 166856 | 19, 37, 3 |
| 12 | RDH5 | retinol dehydrogenase 5 | 135437 | 135441 | 135404 | 1, 25, 14 |
| 12 | RND1 | Rho family GTPase 1 | 172602 | 174243 | 272822 | 21, 1, 8 |
| 12 | ARHGDIB | Rho GDP dissociation inhibitor beta | 111348 | 139055 | 139053 | 7, 41, 30 |
| 12 | ARHGAP9 | Rho GTPase activating protein 9 | 123329 | 111087 | 166986 | 36, 39, 5 |
| 12 | ARHGEF25 | Rho guanine nucleotide exchange factor 25 | 240771 | 178498 | 135502 | 37, 31, 3 |
| 12 | RIMKLB | ribosomal modification protein rimK like family member B | 166532 | 197614 | 166535 | 1, 32, 26 |
| 12 | RPL41 | ribosomal protein L41 | 229117 | 170515 | 135482 | |
| 12 | RPL6 | ribosomal protein L6 | 089009 | 173064 | 179295 | |
| 12 | RPLP0 | ribosomal protein lateral stalk subunit P0 | 089157 | 089154 | 089159 | 43, 7, 21 |
| 12 | RPS26 | ribosomal protein S26 | 197728 | 123411 | 065361 | |
| 12 | RIC8B | RIC8 guanine nucleotide exchange factor B | 111785 | 111783 | 151135 | 18, 17, 45 |
| 12 | RIMBP2 | RIMS binding protein 2 | 060709 | 125207 | 111450 | 29, 28, 20 |
| 12 | RNF10 | ring finger protein 10 | 022840 | 110871 | 167272 | |
| 12 | RNF34 | ring finger protein 34 | 170633 | 089053 | 089094 | 45, 30, 15 |
| 12 | RNF41 | ring finger protein 41 | 181852 | 139613 | 139579 | 43, 2, 9 |
| 12 | RNFT2 | ring finger protein, transmembrane 2 | 135119 | 111412 | 135116 | |
| 12 | RBM19 | RNA binding motif protein 19 | 122965 | 089116 | 089225 | 19, 3, 35 |
| 12 | RBMS2 | RNA binding motif single stranded interacting protein 2 | 076067 | 135423 | 076108 | 40, 32, 37 |
| 12 | RPAP3 | RNA polymerase II associated protein 3 | 005175 | 179715 | 111405 | 29, 18, 13 |
| 12 | POLR3B | RNA polymerase III subunit B | 013503 | 166046 | 111783 | 31, 24, 3 |
| 12 | SARNP | SAP domain containing ribonucleoprotein | 205323 | 135414 | 257390 | 3, 41, 6 |
| 12 | SSPN | sarcospan | 123096 | 123095 | 123104 | |
| 12 | SCARB1 | scavenger receptor class B member 1 | 073060 | 196498 | 150991 | 2, 34, 1 |
| 12 | SCYL2 | SCY1 like pseudokinase 2 | 136021 | 166153 | 179520 | 33, 22, 45 |
| 12 | SUDS3 | SDS3 homolog, SIN3A corepressor complex component | 111707 | 135090 | 139767 | 3, 40, 18 |
| 12 | SELPLG | selectin P ligand | 110876 | 183160 | 110880 | 36, 5, 39 |
| 12 | SRSF9 | serine and arginine rich splicing factor 9 | 111786 | 257218 | 088986 | 34, 11, 35 |
| 12 | SDS | serine dehydratase | 135094 | 151176 | 139410 | 19, 27, 2 |
| 12 | SDSL | serine dehydratase like | 139410 | 135094 | 089116 | 1, 20, 17 |
| 12 | SHMT2 | serine hydroxymethyltransferase 2 | 182199 | 182379 | 185633 | 25, 21, 39 |
| 12 | SRRM4 | serine/arginine repetitive matrix 4 | 139767 | 111707 | 152137 | 26, 25, 8 |
| 12 | STK38L | serine/threonine kinase 38 like | 211455 | 214700 | 029153 | 29, 45, 42 |
| 12 | STRAP | serine/threonine kinase receptor associated protein | 023734 | 151491 | 023697 | 13, 38, 3 |
| 12 | STYK1 | serine/threonine/tyrosine kinase 1 | 060140 | 111196 | 060138 | 24, 31, 33 |
| 12 | SETD1B | SET domain containing 1B | 139718 | 139725 | 158104 | 40, 9, 1 |
| 12 | SH2B3 | SH2B adaptor protein 3 | 111252 | 198324 | 204842 | 36, 45, 6 |
| 12 | STAC3 | SH3 and cysteine rich domain 3 | 185482 | 185633 | 258830 | 25, 15, 18 |
| 12 | SDR9C7 | short chain dehydrogenase/reductase family 9C member 7 | 170426 | 025423 | 139547 | 13, 35, |
| 12 | SPPL3 | signal peptide peptidase like 3 | 157837 | 122971 | 135100 | 26, 29, 4 |
| 12 | STAT2 | signal transducer and activator of transcription 2 | 170581 | 110944 | 175336 | 42, 43, 24 |
| 12 | STAT6 | signal transducer and activator of transcription 6 | 166888 | 166886 | 123384 | 24, 43, 39 |
| 12 | SMCO2 | single-pass membrane protein with coiled-coil domains 2 | 165935 | 029153 | 110841 | 44, 16, 32 |
| 12 | SMCO3 | single-pass membrane protein with coiled-coil domains 3 | 179256 | 182993 | 111339 | 9, 17, 14 |
| 12 | SMUG1 | single-strand-selective monofunctional uracil-DNA glycosylase 1 | 123415 | 172789 | 094916 | |
| 12 | SIRT4 | sirtuin 4 | 089163 | 089159 | 170890 | 29, 15, 17 |
| 12 | SSH1 | slingshot protein phosphatase 1 | 084112 | 110880 | 110887 | 41, 14, 35 |
| 12 | SRGAP1 | SLIT-ROBO Rho GTPase activating protein 1 | 196935 | 118600 | 174206 | |
| 12 | SMAGP | small cell adhesion glycoprotein | 170545 | 183283 | 110934 | 32, 37, 18 |
| 12 | SMIM10L1 | small integral membrane protein 10 like 1 | 256537 | 256188 | 186136 | 40, 25, 26 |
| 12 | SNRPF | small nuclear ribonucleoprotein polypeptide F | 139343 | 074527 | 165972 | 31, 21, 12 |
| 12 | SNRNP35 | small nuclear ribonucleoprotein U11/U12 subunit 35 | 184209 | 150977 | 188026 | 3, 30, 13 |
| 12 | SCNN1A | sodium channel epithelial 1 alpha subunit | 111319 | 067182 | 111321 | 42, 40, 27 |
| 12 | SCN8A | sodium voltage-gated channel alpha subunit 8 | 196876 | 050438 | 167612 | |
| 12 | SLC11A2 | solute carrier family 11 member 2 | 110911 | 214511 | 050426 | 42, 39, 8 |
| 12 | SLC15A4 | solute carrier family 15 member 4 | 139370 | 181234 | 151948 | 22, 40, 39 |
| 12 | SLC15A5 | solute carrier family 15 member 5 | 188991 | 023697 | 008394 | |
| 12 | SLC16A7 | solute carrier family 16 member 7 | 118596 | 139263 | 198673 | 25, 17, 42 |
| 12 | SLC17A8 | solute carrier family 17 member 8 | 179520 | 136021 | 012504 | 6, 39, 16 |
| 12 | SLC2A13 | solute carrier family 2 member 13 | 151229 | 180116 | 188906 | |
| 12 | SLC2A14 | solute carrier family 2 member 14 | 173262 | 111704 | 059804 | 38, 9, 8 |
| 12 | SLC2A3 | solute carrier family 2 member 3 | 059804 | 173262 | 065970 | 42, 39, 38 |
| 12 | SLC25A3 | solute carrier family 25 member 3 | 075415 | 120802 | 166130 | 41, 21, 35 |
| 12 | SLC26A10 | solute carrier family 26 member 10 | 135502 | 240771 | 135454 | 8, 42, 14 |
| 12 | SLC35E3 | solute carrier family 35 member E3 | 175782 | 111581 | 135679 | 2, 4, 13 |
| 12 | SLC38A1 | solute carrier family 38 member 1 | 111371 | 139218 | 134294 | 29, 32, 22 |
| 12 | SLC38A2 | solute carrier family 38 member 2 | 134294 | 111371 | 139209 | 18, 42, 25 |
| 12 | SLC38A4 | solute carrier family 38 member 4 | 139209 | 134294 | 139211 | 19, 4, 28 |
| 12 | SLC39A5 | solute carrier family 39 member 5 | 139540 | 139579 | 139645 | 4, 17, 16 |
| 12 | SLC4A8 | solute carrier family 4 member 8 | 050438 | 139629 | 196876 | 23, 26, 27 |
| 12 | SLC41A2 | solute carrier family 41 member 2 | 136052 | 171310 | 151131 | 13, 10, 2 |
| 12 | SLC48A1 | solute carrier family 48 member 1 | 211584 | 079337 | 061273 | 27, 8, 2 |
| 12 | SLC5A8 | solute carrier family 5 member 8 | 256870 | 151572 | 120800 | 42, 17, 40 |
| 12 | SLC6A12 | solute carrier family 6 member 12 | 111181 | 120645 | 010379 | 17, 19, 8 |
| 12 | SLC6A13 | solute carrier family 6 member 13 | 010379 | 111181 | 073614 | 17, 14, 8 |
| 12 | SLC6A15 | solute carrier family 6 member 15 | 072041 | 179104 | 231738 | 8, 32, 35 |
| 12 | SLC8B1 | solute carrier family 8 member B1 | 089060 | 186815 | 151176 | |
| 12 | SLCO1A2 | solute carrier organic anion transporter family member 1A2 | 084453 | 134538 | 121351 | 8, 18, 42 |
| 12 | SLCO1B1 | solute carrier organic anion transporter family member 1B1 | 134538 | 257062 | 084453 | 19, 36, 23 |
| 12 | SLCO1B3 | solute carrier organic anion transporter family member 1B3 | 111700 | 139155 | 257046 | 19, 37, 7 |
| 12 | SLCO1B7 | solute carrier organic anion transporter family member 1B7 | 205754 | 257046 | 257062 | 19, |
| | | (putative) | | | | |
| 12 | SLCO1C1 | solute carrier organic anion transporter family member 1C1 | 139155 | 172572 | 111700 | 11, 26, 40 |
| 12 | SP1 | Sp1 transcription factor | 185591 | 170374 | 135409 | 22, 29, 12 |
| 12 | SP7 | Sp7 transcription factor | 170374 | 094914 | 185591 | 11, 42, 10 |
| 12 | SPATS2 | spermatogenesis associated serine rich 2 | 123352 | 178401 | 135519 | 45, 40, 4 |
| 12 | SPX | spexin hormone | 134548 | 111711 | 111713 | |
| 12 | SPIC | Spi-C transcription factor | 166211 | 120805 | 196091 | 19, |
| 12 | SPSB2 | splA/ryanodine receptor domain and SOCS box containing 2 | 111671 | 111669 | 010626 | |
| 12 | SFSWAP | splicing factor SWAP homolog | 061936 | 111452 | 198598 | 41, 27, 22 |
| 12 | SPRYD3 | SPRY domain containing 3 | 167778 | 111077 | 167779 | 8, 45, 42 |
| 12 | SPRYD4 | SPRY domain containing 4 | 176422 | 135517 | 135423 | 4, 7, 27 |
| 12 | SART3 | squamous cell carcinoma antigen recognized by T-cells 3 | 075856 | 198855 | 136003 | 18, 38, 15 |
| 12 | SCAF11 | SR-related CTD associated factor 11 | 139218 | 189079 | 111371 | 11, 22, 1 |
| 12 | SOX5 | SRY-box5 | 134532 | 139163 | 060982 | 38, 10, 20 |
| 12 | ST8SIA1 | ST8 alpha-N-acetyl-neuraminide alpha-2,8-sialyltransferase 1 | 111728 | 111726 | 111731 | 29, 11, 34 |
| 12 | STAB2 | stabilin 2 | 136011 | 179088 | 111696 | 36, 18, 24 |
| 12 | SOAT2 | sterol O-acyltransferase 2 | 167780 | 167779 | 139631 | 19, 35, |
| 12 | SBNO1 | strawberry notch homolog 1 | 139697 | 111328 | 183955 | 39, 24, 23 |
| 12 | SUOX | sulfite oxidase | 139531 | 111540 | 123411 | 3, 40, 4 |
| 12 | SENP1 | SUMO1/sentrin specific peptidase 1 | 079387 | 139219 | 152556 | 10, 38, 22 |
| 12 | SOCS2 | suppresser of cytokine signaling 2 | 120833 | 198015 | 169372 | |
| 12 | SVOP | SV2 related protein | 166111 | 110887 | 135093 | 8, 38, 17 |
| 12 | SMARCC2 | SWI/SNF related, matrix associated, actin dependent | 139613 | 092841 | 181852 | 40, 41, 39 |
| | | regulator of chromatin subfamily c member 2 | | | | |
| 12 | SMARCD1 | SWI/SNF related, matrix associated, actin dependent | 066117 | 110881 | 167588 | |
| | | regulator of chromatin, subfamily d, member 1 | | | | |
| 12 | SYCP3 | synaptonemal complex protein 3 | 139351 | 111666 | 111670 | 10, 38, 33 |
| 12 | SYT1 | synaptotagmin 1 | 067715 | 067798 | 177425 | 26, 8, 31 |
| 12 | SYT10 | synaptptagmin 10 | 110975 | 057294 | 139133 | 25, 28, |
| 12 | STX2 | syntaxin 2 | 111450 | 060709 | 132341 | 1, 40, 26 |
| 12 | TAC3 | tachykinin 3 | 166863 | 166860 | 166866 | 32, 10, 37 |
| 12 | TBK1 | TANK binding kinase 1 | 183735 | 184575 | 153179 | 26, 11, 22 |
| 12 | TAOK3 | TAO kinase 3 | 135090 | 089220 | 111707 | 39, 22, 41 |
| 12 | TAPBPL | TAP binding protein like | 139192 | 139193 | 139190 | |
| 12 | TARBP2 | TARBP2, RISC loading complex RNA binding subunit | 139546 | 139625 | 267281 | 1, 34, 22 |
| 12 | TAS2R10 | taste 2 receptor member 10 | 121318 | 231887 | 275778 | 19, |
| 12 | TAS2R13 | taste 2 receptor member 13 | 212128 | 275778 | 134551 | |
| 12 | TAS2R14 | taste 2 receptor member 14 | 212127 | 134551 | 212126 | 22, 45, 42 |
| 12 | TAS2R19 | taste 2 receptor member 19 | 212124 | 255837 | 256436 | |
| 12 | TAS2R20 | taste 2 receptor member 20 | 255837 | 212126 | 212124 | 14, 35, |
| 12 | TAS2R30 | taste 2 receptor member 30 | 256188 | 255374 | 256537 | 30, 17, |
| 12 | TAS2R31 | taste 2 receptor member 31 | 256436 | 212124 | 226761 | |
| 12 | TAS2R42 | taste 2 receptor member 42 | 186136 | 256537 | 197870 | |
| 12 | TAS2R43 | taste 2 receptor member 43 | 255374 | 226761 | 256188 | |
| 12 | TAS2R46 | taste 2 receptor member 46 | 226761 | 256436 | 255374 | |
| 12 | TAS2R50 | taste 2 receptor member 50 | 212126 | 212127 | 255837 | |
| 12 | TAS2R7 | taste 2 receptor member 7 | 121377 | 060138 | 121314 | |
| 12 | TAS2R8 | taste 2 receptor member 8 | 121314 | 121377 | 121381 | |
| 12 | TAS2R9 | taste 2 receptor member 9 | 121381 | 121314 | 231887 | |
| 12 | TBC1D15 | TBC1 domain family member 15 | 121749 | 080371 | 139287 | 39, 38, 24 |
| 12 | TBC1D30 | TBC1 domain family member 30 | 111490 | 135677 | 156076 | 8, 16, 45 |
| 12 | TBX3 | T-box 3 | 135111 | 089225 | 123066 | 2, 32, 33 |
| 12 | TBX5 | T-box 5 | 089225 | 122965 | 135111 | 32, 13, 15 |
| 12 | TCP11L2 | t-complex 11 like 2 | 166046 | 136026 | 013503 | 34, 37, 39 |
| 12 | TEAD4 | TEA domain transcription factor 4 | 197905 | 078246 | 011105 | 9, 37, 13 |
| 12 | TCTN1 | tectonic family member 1 | 204852 | 196850 | 122986 | 41, 1, 26 |
| 12 | TCTN2 | tectonic family member 2 | 168778 | 111358 | 185344 | 45, 17, 16 |
| 12 | TNS2 | tensin 2 | 111077 | 063046 | 167778 | 26, 40, 6 |
| 12 | TESC | tescalcin | 088992 | 174989 | 135108 | 31, 21, 40 |
| 12 | TSPAN11 | tetraspanin 11 | 110900 | 110888 | 013573 | 26, 34, 44 |
| 12 | TSPAN19 | tetraspanin 19 | 231738 | 072041 | 133640 | 10, 42, 30 |
| 12 | TSPAN31 | tetraspanin 31 | 135452 | 135439 | 135446 | 30, 25, 13 |
| 12 | TSPAN8 | tetraspanin 8 | 127324 | 153233 | 139292 | 37, 16, 3 |
| 12 | TSPAN9 | tetraspanin 9 | 011105 | 197905 | 111218 | 34, 14, 29 |
| 12 | THAP2 | THAP domain containing 2 | 173451 | 133858 | 258064 | 22, 45, 25 |
| 12 | TXNRD1 | thioredoxin reductase 1 | 198431 | 120837 | 255150 | 42, 24, 45 |
| 12 | TDG | thymine DNA glyco sylase | 139372 | 204954 | 120820 | 39, 29, 4 |
| 12 | TESPA1 | thymocyte expressed, positive selection associated 1 | 135426 | 172551 | 123307 | 8, 5, 39 |
| 12 | TMPO | thymopoietin | 120802 | 139350 | 075415 | 11, 22, 4 |
| 12 | TRHDE | thyrotropin releasing hormone degrading enzyme | 072657 | 139287 | 253719 | 25, 4, 42 |
| 12 | TIMELESS | timeless circadian clock | 111602 | 175336 | 135517 | 3, 41, 33 |
| 12 | TNFRSF1A | TNF receptor superfamily member 1A | 067182 | 008323 | 111319 | 36, 2, 45 |
| 12 | TIGAR | TP53 induced glycolysis regulatory phosphatase | 078237 | 118971 | 118972 | |
| 12 | TRIAP1 | TP53 regulated inhibitor of apoptosis 1 | 170855 | 111780 | 257218 | 34, 43, 27 |
| 12 | TRAFD1 | TRAF-type zinc finger domain containing 1 | 135148 | 111300 | 173064 | 41, 34, 36 |
| 12 | TFCP2 | transcription factor CP2 | 135457 | 110925 | 184271 | 15, 42, 10 |
| 12 | TRPV4 | transient receptor potential cation channel subfamily V member 4 | 111199 | 139438 | 139433 | 31, 24, 35 |
| 12 | TM7SF3 | transmembrane 7 superfamily member 3 | 064115 | 111790 | 152944 | 5, 4, 3 |
| 12 | TMCC3 | transmembrane and coiled-coil domain family 3 | 057704 | 173588 | 184752 | |
| 12 | TMTC1 | transmembrane and tetratricopeptide repeat containing 1 | 133687 | 187950 | 133704 | 26, 31, 25 |
| 12 | TMTC2 | transmembrane and tetratricopeptide repeat containing 2 | 179104 | 127720 | 072041 | 33, 1, 24 |
| 12 | TMTC3 | transmembrane and tetratricopeptide repeat containing 3 | 139324 | 198707 | 049130 | 11, 4, 17 |
| 12 | TMBIM4 | transmembrane BAX inhibitor motif containing 4 | 155957 | 228144 | 090376 | 1, 30, 10 |
| 12 | TMBIM6 | transmembrane BAX inhibitor motif containing 6 | 139644 | 161791 | 167566 | 24, 30, 42 |
| 12 | TMED2 | transmembrane p24 trafficking protein 2 | 086598 | 188026 | 111364 | 4, 31, 13 |
| 12 | TMPRSS12 | transmembrane protease, serine 12 | 186452 | 123268 | 185432 | 38, 32, 8 |
| 12 | TMEM106C | transmembrane protein 106C | 134291 | 111424 | 139219 | 9, 29, 4 |
| 12 | TMEM116 | transmembrane protein 116 | 198270 | 089022 | 089248 | 29, 34, 43 |
| 12 | TMEM117 | transmembrane protein 117 | 139173 | 151239 | 184613 | 11, 26, 35 |
| 12 | TMEM119 | transmembrane protein 119 | 183160 | 136003 | 110876 | |
| 12 | TMEM120B | transmembrane protein 120B | 188735 | 139714 | 139725 | |
| 12 | TMEM132B | transmembrane protein 132B | 139364 | 081760 | 255595 | 26, 8, 40 |
| 12 | TMEM132C | transmembrane protein 132C | 181234 | 255595 | 139370 | 11, 1, 28 |
| 12 | TMEM132D | transmembrane protein 132D | 151952 | 151948 | 111432 | 26, 28, 14 |
| 12 | TMEM19 | transmembrane protein 19 | 139291 | 258064 | 080371 | 22, 43, 4 |
| 12 | TMEM233 | transmembrane protein 233 | 224982 | 183273 | 111725 | 40, 6, 25 |
| 12 | TMEM263 | transmembrane protein 263 | 151135 | 111785 | 120832 | 45, 42, 29 |
| 12 | TMEM5 | transmembrane protein 5 | 118600 | 177990 | 196935 | 26, 9, 6 |
| 12 | TMEM52B | transmembrane protein 52B | 165685 | 173391 | 139112 | 17, 32, 5 |
| 12 | TCHP | trichoplein keratin filament binding | 139437 | 139433 | 139436 | 41, 17, 3 |
| 12 | TPI1 | triosephosphate isomerase 1 | 111669 | 111667 | 111671 | 30, 7, 34 |
| 12 | TROAP | trophinin associated protein | 135451 | 135406 | 186897 | 39, 9, 21 |
| 12 | TPH2 | tryptophan hydroxylase 2 | 139287 | 121749 | 072657 | 38, 8, |
| 12 | TSFM | Ts translation elongation factor, mitochondrial | 123297 | 257921 | 135407 | 33, 9, 1 |
| 12 | TULP3 | tubby like protein 3 | 078246 | 171792 | 197905 | 27, 18, 9 |
| 12 | TUBA1A | tubulin alpha 1a | 167552 | 123416 | 167553 | 8, 30, 26 |
| 12 | TUBA1B | tubulin alpha 1b | 123416 | 139636 | 167552 | 21, 3, 9 |
| 12 | TUBA1C | tubulin alpha 1c | 167553 | 167552 | 135406 | |
| 12 | TWF1 | twinfilin actin binding protein 1 | 151239 | 198001 | 139173 | 4, 22, 33 |
| 12 | TPCN1 | two pore segment channel 1 | 186815 | 166578 | 089060 | 40, 41, 22 |
| 12 | YARS2 | tyrosyl-tRNA synthetase 2 | 139131 | 087470 | 057294 | 12, 9, 3 |
| 12 | UBC | ubiquitin C | 150991 | 073060 | 150990 | 36, 39, 42 |
| 12 | UBE2N | ubiquitin conjugating enzyme E2 N | 177889 | 173598 | 198015 | 31, 41, 30 |
| 12 | UBE3B | ubiquitin protein ligase E3B | 151148 | 110906 | 139428 | 29, 22, 34 |
| 12 | USP15 | ubiquitin specific peptidase 15 | 135655 | 198673 | 061987 | 5, 22, 11 |
| 12 | USP30 | ubiquitin specific peptidase 30 | 135093 | 166111 | 189046 | 2, 13, 29 |
| 12 | USP44 | ubiquitin specific peptidase 44 | 136014 | 111142 | 074527 | 38, 12, 22 |
| 12 | USP5 | ubiquitin specific peptidase 5 | 111667 | 111665 | 111669 | 21, 26, 34 |
| 12 | B3GNT4 | UDP-GlcNAc: betaGal beta-1,3-N-acetylglucosaminyltransferase 4 | 176383 | 204671 | 184047 | 3, 17, 20 |
| 12 | UHRF1BP1L | UHRF1 binding protein 1 like | 111647 | 180219 | 075089 | 6, 38, 34 |
| 12 | UNC119B | unc-119 lipid binding chaperone B | 175970 | 110917 | 122971 | 34, 29, 22 |
| 12 | ULK1 | unc-51 like autophagy activating kinase 1 | 177169 | 198598 | 177192 | 27, 3, 29 |
| 12 | UNG | uracil DNA glycosylase | 076248 | 189046 | 076555 | 2, 21, 9 |
| 12 | UTP20 | UTP20, small subunit processome component | 120800 | 256870 | 120805 | |
| 12 | VAMP1 | vesicle associated membrane protein 1 | 139190 | 139192 | 111639 | 39, 26, 36 |
| 12 | VEZT | vezatin, adherens junctions transmembrane protein | 028203 | 180263 | 111142 | 13, 24, 39 |
| 12 | VDR | vitamin D (1,25-dihydroxyvitamin D3) receptor | 111424 | 061273 | 134291 | 29, 27, 24 |
| 12 | VWF | von Willebrand factor | 110799 | 047617 | 010278 | 43, 1, 26 |
| 12 | VPS29 | VPS29, retromer complex component | 111237 | 204856 | 151164 | 29, 4, 43 |
| 12 | VPS33A | VPS33A, CORVET/HOPS core subunit | 139719 | 256861 | 130779 | 37, 1, 3 |
| 12 | VPS37B | VPS37B, ESCRT-I subunit | 139722 | 130787 | 150967 | 28, 14, 25 |
| 12 | VSIG10 | V-set and immunoglobulin domain containing 10 | 176834 | 176871 | 089220 | |
| 12 | WSB2 | WD repeat and SOCS box containing 2 | 176871 | 111445 | 176834 | 18, 34, 43 |
| 12 | WDR66 | WD repeat domain 66 | 158023 | 256950 | 110987 | 36, 30, 38 |
| 12 | WNK1 | WNK lysine deficient protein kinase 1 | 060237 | 171840 | 002016 | 29, 22, 24 |
| 12 | WNT1 | Wnt family member 1 | 125084 | 169884 | 181418 | 38, |
| 12 | WNT10B | Wnt family member 10B | 169884 | 134287 | 125084 | 18, 26, 15 |
| 12 | WNT5B | Wnt family member 5B | 111186 | 082805 | 171823 | 25, 33, 7 |
| 12 | WIF1 | WNT inhibitory factor 1 | 156076 | 111490 | 174106 | 40, 31, 14 |
| 12 | WSCD2 | WSC domain containing 2 | 075035 | 187855 | 174600 | |
| 12 | WBP11 | WW domain binding protein 11 | 084463 | 246705 | 182993 | 38, 9, 40 |
| 12 | YBX3 | Y-box binding protein 3 | 060138 | 060140 | 121377 | 25, 5, 14 |
| 12 | YEATS4 | YEATS domain containing 4 | 127337 | 090382 | 166225 | 22, 10, 38 |
| 12 | YAF2 | YY1 associated factor 2 | 015153 | 151233 | 134283 | 11, 26, 2 |
| 12 | ZBTB39 | zinc finger and BTB domain containing 39 | 166860 | 166856 | 166863 | 1, 2, 38 |
| 12 | ZFC3H1 | zinc finger C3H1 -type containing | 133858 | 139292 | 173451 | 26, 1, 22 |
| 12 | ZC3H10 | zinc finger CCCH-type containing 10 | 135482 | 229117 | 139641 | 2, 39, 13 |
| 12 | ZCRB1 | zinc finger CCHC-type and RNA binding motif containing 1 | 139168 | 134283 | 139174 | 43, 29, 22 |
| 12 | ZCCHC8 | zinc finger CCHC-type containing 8 | 033030 | 130779 | 111011 | 11, 21, 40 |
| 12 | ZDHHC17 | zinc finger DHHC-type containing 17 | 186908 | 091039 | 175183 | 18, 11, 2 |
| 12 | ZNF10 | zinc finger protein 10 | 256223 | 214029 | 256825 | 1, 29, 26 |
| 12 | ZNF140 | zinc finger protein 140 | 196387 | 198040 | 214029 | 40, 1, 4 |
| 12 | ZNF26 | zinc finger protein 26 | 198393 | 196458 | 198040 | 42, 18, 24 |
| 12 | ZNF268 | zinc finger protein 268 | 090612 | 256825 | 227059 | 42, 11, 38 |
| 12 | ZNF384 | zinc finger protein 384 | 126746 | 111653 | 139200 | 43, 18, 39 |
| 12 | ZNF385A | zinc finger protein 385A | 161642 | 139572 | 161638 | 7, 14, 9 |
| 12 | ZNF605 | zinc finger protein 605 | 196458 | 072609 | 198393 | 40, 22, 1 |
| 12 | ZNF641 | zinc finger protein 641 | 167528 | 187166 | 139223 | 31, 2, 43 |
| 12 | ZNF664 | zinc finger protein 664 | 179195 | 250091 | 178882 | 11, 29, 26 |
| 12 | ZNF705A | zinc finger protein 705A | 196946 | 111729 | 171847 | 38, 10, |
| 12 | ZNF740 | zinc finger protein 740 | 139651 | 139631 | 139626 | 33, |
| 12 | ZNF84 | zinc finger protein 84 | 198040 | 198393 | 196387 | |
| 12 | ZNF891 | zinc finger protein 891 | 214029 | 196387 | 256223 | 19, 32, 20 |
| 13 | HTR2A | 5-hydroxytryptamine receptor 2A | 102468 | 139684 | 136143 | 42, 27, 8 |
| 13 | ABHD13 | abhydrolase domain containing 13 | 139826 | 174405 | 102524 | 9, 22, 2 |
| 13 | ACOD1 | aconitate decarboxylase 1 | 102794 | 178695 | 102805 | 5, 20, |
| 13 | ARL11 | ADP ribosylation factor like GTPase 11 | 152213 | 136144 | 123179 | 21, 7, 45 |
| 13 | ADPRHL1 | ADP-ribosylhydrolase like 1 | 153531 | 139835 | 150401 | 21, 6, 25 |
| 13 | AKAP11 | A-kinase anchoring protein 11 | 023516 | 102780 | 120659 | 26, 25, 45 |
| 13 | ALG11 | ALG11, alpha-1,2-mannosyltransferase | 253710 | 123191 | 253797 | 22, 40, 43 |
| 13 | ALG5 | ALG5, dolichyl-phosphate beta-glucosyltransferase | 120697 | 120693 | 120699 | 29, 26, 17 |
| 13 | ANKRD10 | ankyrin repeat domain 10 | 088448 | 153487 | 102606 | 11, 26, 37 |
| 13 | AMER2 | APC membrane recruitment protein 2 | 165566 | 151846 | 139505 | 26, 14, 31 |
| 13 | ALOX5AP | arachidonate 5-lipoxygenase activating protein | 132965 | 132952 | 102802 | 30, 5, 7 |
| 13 | ARGLU1 | arginine and glutamate rich 1 | 134884 | 125266 | 204442 | 29, 1, 31 |
| 13 | ABCC4 | ATP binding cassette subfamily C member 4 | 125257 | 125285 | 134873 | 33, 37, 29 |
| 13 | ATP5EP2 | ATP synthase, H+ transporting, mitochondrial F1 complex, | 180389 | 139515 | 165556 | 33, 30, 9 |
| | | epsilon subunit pseudogene 2 | | | | |
| 13 | ATP7B | ATPase copper transporting beta | 123191 | 123171 | 253710 | |
| 13 | ATP4B | ATPase H+/K+ transporting beta subunit | 186009 | 198176 | 185974 | 37, |
| 13 | ATP12A | ATPase H+/K+ transporting non-gastric alpha2 subunit | 075673 | 102699 | 132972 | 42, 32, 39 |
| 13 | ATP11A | ATPase phospholipid transporting 11A | 068650 | 126216 | 126217 | 29, 1, 3 |
| 13 | ATP8A2 | ATPase phospholipid transporting 8A2 | 132932 | 139496 | 180730 | 31, 20, 26 |
| 13 | BIVM | basic, immunoglobulin-like variable motif containing | 134897 | 134901 | 270181 | 7, 45, 27 |
| 13 | B3GLCT | beta 3-glucosyltransferase | 187676 | 120694 | 133105 | 11, 14, 4 |
| 13 | BIVM-ERCC5 | BIVM-ERCC5 readthrough | 270181 | 134897 | 134899 | |
| 13 | BORA | bora, aurora kinase A activator | 136122 | 204899 | 083520 | 4, 30, 6 |
| 13 | BRCA2 | BRCA2, DNA repair associated | 139618 | 189167 | 139597 | 22, 4, 23 |
| 13 | C1QTNF9 | C1q and tumor necrosis factor related protein 9 | 240654 | 182957 | 281899 | 30, 6, 38 |
| 13 | C1QTNF9B | C1q and tumor necrosis factor related protein 9B | 205863 | 027001 | 273167 | 38, |
| 13 | C1QTNF9B-AS1 | C1QTNF9B antisense RNA 1 | 281899 | 240654 | 102699 | 38, 18, 33 |
| 13 | CAB39L | calcium binding protein 39 like | 102547 | 102543 | 136169 | 29, 34, 38 |
| 13 | CPB2 | carboxypeptidase B2 | 080618 | 123200 | 136167 | 19, 23, 36 |
| 13 | CSNK1A1L | casein kinase 1 alpha 1 like | 180138 | 279238 | 133110 | 38, |
| 13 | CDX2 | caudal type homeobox 2 | 165556 | 180389 | 183463 | 16, 3, 19 |
| 13 | CCDC169- | CCDC169-SOHLH2 readthrough | 250709 | 120669 | 242715 | |
| | SOHLH2 | | | | | |
| 13 | CDC16 | cell division cycle 16 | 130177 | 283361 | 169062 | 31, 26, 2 |
| 13 | CENPJ | centromere protein J | 151849 | 132972 | 151846 | 1, 11, 31 |
| 13 | CLN5 | ceroid-lipofuscinosis, neuronal 5 | 102805 | 102794 | 283208 | 24, 42, 26 |
| 13 | CHAMP1 | chromosome alignment maintaining phosphoprotein 1 | 198824 | 169062 | 133083 | |
| 13 | CLYBL | citrate lyase beta like | 125246 | 125304 | 139800 | 17, 6, 23 |
| 13 | CLDN10 | claudin 10 | 134873 | 125257 | 134874 | 30, 17, 34 |
| 13 | F7 | coagulation factor VII | 057593 | 126217 | 126218 | 19, 23, 38 |
| 13 | F10 | coagulation factor X | 126218 | 057593 | 126231 | 30, 1, 33 |
| 13 | CCDC122 | coiled-coil domain containing 122 | 151773 | 120658 | 179630 | 4, 34, 22 |
| 13 | CCDC168 | coiled-coil domain containing 168 | 175820 | 139780 | 151287 | 38, 19, 8 |
| 13 | CCDC169 | coiled-coil domain containing 169 | 242715 | 250709 | 133104 | 38, 7, 45 |
| 13 | CCDC70 | coiled-coil domain containing 70 | 123171 | 281106 | 123191 | 38, 10, |
| 13 | COL4A1 | collagen type IV alpha 1 chain | 187498 | 185950 | 134871 | 11, 1, 12 |
| 13 | COL4A2 | collagen type IV alpha 2 chain | 134871 | 187498 | 139832 | 1, 26, 12 |
| 13 | COMMD6 | COMM domain containing 6 | 188243 | 136111 | 118939 | 15, 11, 7 |
| 13 | COG3 | component of oligomeric golgi complex 3 | 136152 | 174032 | 165837 | 42, 13, 26 |
| 13 | COG6 | component of oligomeric golgi complex 6 | 133103 | 183722 | 150907 | 42, 29, 24 |
| 13 | CRYL1 | crystallin lambda 1 | 165475 | 121742 | 032742 | 31, 1, 35 |
| 13 | CUL4A | cullin 4A | 139842 | 126226 | 185896 | 9, 18, 6 |
| 13 | CCNA1 | cyclin A1 | 133101 | 133104 | 180440 | 7, 38, 29 |
| 13 | CDK8 | cyclin dependent kinase 8 | 132964 | 127870 | 132970 | 34, 43, 11 |
| 13 | CYSLTR2 | cysteinyl leukotriene receptor 2 | 152207 | 136161 | 102531 | 32, 25, 10 |
| 13 | CARS2 | cysteinyl-tRNA synthetase 2, mitochondrial (putative) | 134905 | 213995 | 153487 | 5, 21, 38 |
| 13 | CDADC1 | cytidine and dCMP deaminase domain containing 1 | 102543 | 102539 | 102547 | 43, 24, 19 |
| 13 | CKAP2 | cytoskeleton associated protein 2 | 136108 | 136100 | 139675 | 4, 22, 38 |
| 13 | DACH1 | dachshund family transcription factor 1 | 276644 | 150361 | 204899 | 14, 36, 44 |
| 13 | DAOA | D-amino acid oxidase activator | 182346 | 125255 | 125266 | |
| 13 | DZIP1 | DAZ interacting zinc finger protein 1 | 134874 | 134873 | 102580 | 10, 6, 38 |
| 13 | DOCK9 | dedicator of cytokinesis 9 | 088387 | 088386 | 134882 | 33, 1, 32 |
| 13 | DCUN1D2 | defective in cullin neddylation 1 domain containing 2 | 150401 | 153531 | 150403 | 39, 1, 40 |
| 13 | DHRS12 | dehydrogenase/reductase 12 | 102796 | 139668 | 281106 | 1, 13, 14 |
| 13 | DLEU1 | Deleted in lymphocytic leukemia 1 | 176124 | 198553 | 186047 | |
| 13 | DLEU7 | deleted in lymphocytic leukemia, 7 | 186047 | 176124 | 136104 | 39, 8, 16 |
| 13 | DGKH | diacylglycerol kinase eta | 102780 | 102763 | 023516 | 30, 4, 27 |
| 13 | DIAPH3 | diaphanous related formin 3 | 139734 | 118946 | 083544 | 10, 38, 6 |
| 13 | DIS3 | DIS3 homolog, exosome endoribonuclease and 3′-5′ | 083520 | 136122 | 083535 | 24, 42, 38 |
| | | exoribonuclease | | | | |
| 13 | LIG4 | DNA ligase 4 | 174405 | 204442 | 139826 | 39, 11, 31 |
| 13 | DNAJC15 | DnaJ heat shock protein family (Hsp40) member C15 | 120675 | 133106 | 120658 | 10, 15, 31 |
| 13 | DNAJC3 | DnaJ heat shock protein family (Hsp40) member C3 | 102580 | 134874 | 102595 | 13, 29, 42 |
| 13 | DCT | dopachrome tautomerase | 080166 | 183098 | 088451 | 35, 11, 14 |
| 13 | DCLK1 | doublecortin like kinase 1 | 133083 | 180660 | 120669 | 8, 31, 1 |
| 13 | ELF1 | E74 like ETS transcription factor 1 | 120690 | 102743 | 120688 | 42, 22, 39 |
| 13 | ENOX1 | ecto-NOX disulfide-thiol exchanger 1 | 120658 | 120675 | 151773 | 38, 45, 17 |
| 13 | EBPL | emopamil binding protein like | 123179 | 152213 | 102753 | 11, 17, 7 |
| 13 | EDNRB | endothelin receptor type B | 136160 | 139737 | 152192 | 32, 11, 26 |
| 13 | EFNB2 | ephrin B2 | 125266 | 182346 | 134884 | 11, 1, 15 |
| 13 | EPSTI1 | epithelial stromal interaction 1 | 133106 | 179813 | 120675 | 18, 39, 37 |
| 13 | ERCC5 | ERCC excision repair 5, endonuclease | 134899 | 270181 | 125255 | 42, 22, 3 |
| 13 | ESD | esterase D | 139684 | 136141 | 102468 | 29, 41, 11 |
| 13 | EEF1AKMT1 | eukaryotic translation elongation factor 1 alpha lysine | 150456 | 172458 | 132953 | |
| | | methyltransferase 1 | | | | |
| 13 | EXOSC8 | exosome component 8 | 120699 | 120697 | 102710 | 34, 4, 29 |
| 13 | XPO4 | exportin 4 | 132953 | 150456 | 150457 | 14, 22, 25 |
| 13 | FAM124A | family with sequence similarity 124 member A | 150510 | 226792 | 253309 | |
| 13 | FAM155A | family with sequence similarity 155 member A | 204442 | 134884 | 174405 | 11, 45, 35 |
| 13 | FAM216B | family with sequence similarity 216 member B | 179813 | 120659 | 133106 | 42, 30, 20 |
| 13 | FBXL3 | F-box and leucine rich repeat protein 3 | 005812 | 283208 | 005810 | 42, 1, 24 |
| 13 | FARP1 | FERM, ARH/RhoGEF and pleckstrin domain protein 1 | 152767 | 065150 | 139797 | |
| 13 | FGF14 | fibroblast growth factor 14 | 102466 | 198542 | 134900 | 20, 40, 26 |
| 13 | FGF9 | fibroblast growth factor 9 | 102678 | 165487 | 279282 | 40, 17, 5 |
| 13 | FNDC3A | fibronectin type III domain containing 3A | 102531 | 152207 | 102539 | 11, 32, 4 |
| 13 | FLT1 | fms related tyrosine kinase 1 | 102755 | 152520 | 132963 | 32, 29, 1 |
| 13 | FLT3 | fms related tyrosine kinase 3 | 122025 | 183463 | 152520 | 7, 4, 5 |
| 13 | FOXO1 | forkhead box O1 | 150907 | 133103 | 102738 | 11, 22, 27 |
| 13 | FREM2 | FRAS1 related extracellular matrix protein 2 | 150893 | 120686 | 133115 | 12, 26, 24 |
| 13 | FRY | FRY microtubule binding protein | 073910 | 133105 | 189167 | 1, 13, 11 |
| 13 | GPR12 | G protein-coupled receptor 12 | 132975 | 132970 | 152484 | 26, 8, 14 |
| 13 | GPR18 | G protein-coupled receptor 18 | 125245 | 134882 | 169508 | 22, 30, 21 |
| 13 | GPR180 | G protein-coupled receptor 180 | 152749 | 088451 | 125285 | 9, 45, 18 |
| 13 | GPR183 | G protein-coupled receptor 183 | 169508 | 125245 | 125304 | 30, 5, 42 |
| 13 | GRK1 | G protein-coupled receptor kinase 1 | 185974 | 186009 | 184497 | |
| 13 | GGACT | gamma-glutamylamine cyclotransferase | 134864 | 175198 | 125247 | 17, 22, 29 |
| 13 | GJA3 | gap junction protein alpha 3 | 121743 | 121741 | 165474 | 14, 17, 15 |
| 13 | GJB2 | gap junction protein beta 2 | 165474 | 121743 | 121742 | 30, 11, 24 |
| 13 | GJB6 | gap junction protein beta 6 | 121742 | 165474 | 165475 | 30, 24, 4 |
| 13 | GTF2F2 | general transcription factor IIF subunit 2 | 188342 | 133114 | 180332 | 1, 37, 12 |
| 13 | GTF3A | general transcription factor IIIA | 122034 | 122035 | 122033 | 9, 41, 21 |
| 13 | ERICH6B | glutamate rich 6B | 165837 | 136152 | 174015 | 2, 38, 28 |
| 13 | GPC5 | glypican 5 | 179399 | 165300 | 183098 | |
| 13 | GPC6 | glypican 6 | 183098 | 179399 | 080166 | 11, 12, 10 |
| 13 | GPALPP1 | GPALPP motifs containing 1 | 133114 | 083635 | 188342 | |
| 13 | GAS6 | growth arrest specific 6 | 183087 | 184497 | 283199 | 6, 26, 20 |
| 13 | GRTP1 | growth hormone regulated TBC protein 1 | 139835 | 185896 | 153531 | 34, 17, 27 |
| 13 | GSX1 | GS homeobox 1 | 169840 | 186184 | 139515 | |
| 13 | HSPH1 | heat shock protein family H (Hsp110) member 1 | 120694 | 175664 | 187676 | 31, 42, 26 |
| 13 | HS6ST3 | heparan sulfate 6-O-sulfotransferase 3 | 185352 | 102595 | 165621 | 10, 8, 17 |
| 13 | HNRNPA1L2 | heterogeneous nuclear ribonucleoprotein A1-like 2 | 139675 | 136108 | 165416 | 39, 41, 30 |
| 13 | HMGB1 | high mobility group box 1 | 189403 | 236444 | 132952 | 29, 41, 30 |
| 13 | IPO5 | importin 5 | 065150 | 125249 | 152767 | 40, 38, 42 |
| 13 | ING1 | inhibitor of growth family member 1 | 153487 | 134905 | 088448 | 13, 1, 36 |
| 13 | IRS2 | insulin receptor substrate 2 | 185950 | 041515 | 187498 | 31, 27, 2 |
| 13 | ITM2B | integral membrane protein 2B | 136156 | 136146 | 139687 | 40, 31, 11 |
| 13 | INTS6 | integrator complex subunit 6 | 102786 | 253309 | 139668 | 42, 38, 1 |
| 13 | ITGBL1 | integrin subunit beta like 1 | 198542 | 102452 | 102466 | 11, 45, 10 |
| 13 | IL17D | interleukin 17D | 172458 | 032742 | 150456 | 15, 25, 8 |
| 13 | IFT88 | intraflagellar transport 88 | 032742 | 165475 | 172458 | 38, 31, 30 |
| 13 | KPNA3 | karyopherin subunit alpha 3 | 102753 | 123179 | 123178 | 4, 25, 38 |
| 13 | KATNAL1 | katanin catalytic subunit A1 like 1 | 102781 | 122042 | 236444 | |
| 13 | KDELC1 | KDEL motif containing 1 | 134901 | 151287 | 134897 | 43, 9, 45 |
| 13 | KLHL1 | kelch like family member 1 | 150361 | 184226 | 276644 | 9, 8, 2 |
| 13 | KBTBD6 | kelch repeat and BTB domain containing 6 | 165572 | 120688 | 120696 | 8, 24, 39 |
| 13 | KBTBD7 | kelch repeat and BTB domain containing 7 | 120696 | 165572 | 120662 | 24, 8, 39 |
| 13 | KL | klotho | 133116 | 083642 | 133121 | 29, 17, 37 |
| 13 | KLF12 | Kruppel like factor 12 | 118922 | 102554 | 136111 | 22, 1, 31 |
| 13 | KLF5 | Kruppel like factor 5 | 102554 | 083535 | 118922 | 18, 37, 16 |
| 13 | LACC1 | laccase domain containing 1 | 179630 | 151773 | 281883 | 2, 31, 45 |
| 13 | LATS2 | large tumor suppressor kinase 2 | 150457 | 132953 | 150459 | 11, 45, 37 |
| 13 | LRRC63 | leucine rich repeat containing 63 | 173988 | 136167 | 102445 | 10, 38, 14 |
| 13 | LRCH1 | leucine rich repeats and calponin homology domain containing 1 | 136141 | 102445 | 139684 | 22, 11, 39 |
| 13 | LECT1 | Leukocyte cell derived chemotaxin 1 | 136110 | 165416 | 136099 | 11, 20, 12 |
| 13 | LNX2 | ligand of numb-protein X 2 | 139517 | 122033 | 186184 | 4, 42, 1 |
| 13 | LMO7 | UM domain 7 | 136153 | 118939 | 178734 | 42, 24, 11 |
| 13 | LHFP | lipoma HMGIC fusion partner | 183722 | 188811 | 133103 | 11, 13, 43 |
| 13 | LMO7DN | LMO7 downstream neighbor | 178734 | 136153 | 178695 | 26, 23, 25 |
| 13 | LINC00371 | Long intergenic non-protein coding RNA 371 | 226792 | 136104 | 150510 | 31, 38, 12 |
| 13 | LCP1 | lymphocyte cytosolic protein 1 | 136167 | 080618 | 173988 | 39, 21, 41 |
| 13 | LPAR6 | lysophosphatidic acid receptor 6 | 139679 | 139687 | 136161 | |
| 13 | LAMP1 | lysosomal associated membrane protein 1 | 185896 | 139842 | 139835 | 4, 1, 26 |
| 13 | MAB21L1 | mab-21 like 1 | 180660 | 172915 | 133083 | 2, 14, 24 |
| 13 | MCF2L | MCF.2 cell line derived transforming sequence like | 126217 | 068650 | 057593 | |
| 13 | MED4 | mediator complex subunit 4 | 136146 | 136159 | 136156 | 4, 7, 30 |
| 13 | MEDAG | mesenteric estrogen dependent adipogenesis | 102802 | 132965 | 175664 | 45, 10, 44 |
| 13 | METTL21C | methyltransferase like 21C | 139780 | 134900 | 175820 | 15, 10, 32 |
| 13 | MTUS2 | microtubule associated tumor suppressor candidate 2 | 132938 | 139508 | 139514 | 15, 20, 42 |
| 13 | MICU2 | mitochondrial calcium uptake 2 | 165487 | 180776 | 102678 | 29, 11, 22 |
| 13 | MIPEP | mitochondrial intermediate peptidase | 027001 | 127863 | 205863 | 30, 23, 29 |
| 13 | MRPL57 | mitochondrial ribosomal protein L57 | 173141 | 165480 | 180776 | |
| 13 | MRPS31 | mitochondrial ribosomal protein S31 | 102738 | 150907 | 102743 | 4, 7, 10 |
| 13 | MTRF1 | mitochondrial translation release factor 1 | 120662 | 120696 | 172766 | 28, |
| 13 | MTIF3 | mitochondrial translational initiation factor 3 | 122033 | 122034 | 139517 | 29, 30, 38 |
| 13 | MZT1 | mitotic spindle organizing protein 1 | 204899 | 276644 | 136122 | 1, 25, 4 |
| 13 | MLNR | motilin receptor | 102539 | 102531 | 102543 | 27, |
| 13 | MPHOSPH8 | M-phase phosphoprotein 8 | 196199 | 132958 | 121390 | 16, 18, 33 |
| 13 | MBNL2 | muscleblind like splicing regulator 2 | 139793 | 165621 | 125249 | 22, 4, 2 |
| 13 | MYCBP2 | MYC binding protein 2, E3 ubiquitin protein ligase | 005810 | 005812 | 136155 | 41, 1, 22 |
| 13 | MYO16 | myosin XVI | 041515 | 283384 | 185950 | 26, 10, 7 |
| 13 | MTMR6 | myotubularin related protein 6 | 139505 | 165566 | 139496 | 42, 38, 22 |
| 13 | NAA16 | N(alpha)-acetyltransferase 16, NatA auxiliary subunit | 172766 | 120662 | 102760 | 42, 39, 18 |
| 13 | NAXD | NAD(P)HX dehydratase | 213995 | 139832 | 134905 | 24, 39, 36 |
| 13 | N4BP2L1 | NEDD4 binding protein 2 like 1 | 139597 | 139618 | 244754 | 7, 29, 22 |
| 13 | N4BP2L2 | NEDD4 binding protein 2 like 2 | 244754 | 139597 | 083642 | 40, 29, 18 |
| 13 | NDFIP2 | Nedd4 family interacting protein 2 | 102471 | 139746 | 136158 | 13, 4, 26 |
| 13 | NBEA | neurobeachin | 172915 | 133119 | 180660 | |
| 13 | NHLRC3 | NHL repeat containing 3 | 188811 | 120685 | 183722 | 42, 27, 2 |
| 13 | NEK3 | NIMA related kinase 3 | 136098 | 197168 | 136114 | 11, 13, 14 |
| 13 | NEK5 | NIMA related kinase 5 | 197168 | 253797 | 136098 | 30, 38, 42 |
| 13 | NUP58 | nucleoporin 58 | 139496 | 139505 | 132932 | 38, 30, 22 |
| 13 | NUDT15 | nudix hydrolase 15 | 136159 | 136143 | 136146 | 12, 29, 44 |
| 13 | NUFIP1 | NUFIP1, FMR1 interacting protein 1 | 083635 | 102804 | 133114 | 34, 42, 38 |
| 13 | OLFM4 | olfactomedin 4 | 102837 | 136099 | 204919 | 4, 16, 44 |
| 13 | OXGR1 | oxoglutarate receptor 1 | 165621 | 185352 | 139793 | 42, 40, 24 |
| 13 | PAN3 | PAN3 poly(A) specific ribonuclease subunit | 152520 | 122025 | 102755 | 11, 31, 22 |
| 13 | PDX1 | pancreatic and duodenal homeobox 1 | 139515 | 169840 | 180389 | 28, 3, 37 |
| 13 | PSPC1 | paraspeckle component 1 | 121390 | 196199 | 132950 | 21, 22, 26 |
| 13 | PCID2 | PCI domain containing 2 | 126226 | 126231 | 139842 | 13, 18, 38 |
| 13 | PDS5B | PDS5 cohesin associated factor B | 083642 | 244754 | 133116 | 1, 29, 42 |
| 13 | POSTN | periostin | 133110 | 180138 | 133107 | |
| 13 | PHF11 | PHD finger protein 11 | 136147 | 136169 | 136144 | 18, 37, 41 |
| 13 | PABPC3 | poly(A) binding protein cytoplasmic 3 | 151846 | 151849 | 165566 | 23, 38, 18 |
| 13 | PARP4 | poly(ADP-ribose) polymerase family member 4 | 102699 | 281899 | 075673 | 40, 16, 4 |
| 13 | KCNRG | potassium channel regulator | 198553 | 204977 | 176124 | 42, 30, 15 |
| 13 | KCTD12 | potassium channel tetramerization domain containing 12 | 178695 | 178734 | 102794 | 11, 26, 1 |
| 13 | KCTD4 | potassium channel tetramerization domain containing 4 | 180332 | 188342 | 133112 | 31, 35, 6 |
| 13 | POU4F1 | POU class 4 homeobox 1 | 152192 | 136160 | 152193 | 14, 22, 12 |
| 13 | PIBF1 | progesterone immunomodulatory binding factor 1 | 083535 | 083520 | 102554 | 10, 34, 11 |
| 13 | PROSER1 | proline and serine rich 1 | 120685 | 133115 | 188811 | 31, 12, 44 |
| 13 | PRR20A | proline rich 20A | 204919 | 102837 | 204918 | |
| 13 | PRR20B | proline rich 20B | 204918 | 204919 | 227151 | |
| 13 | PRR20C | proline rich 20C | 229665 | 102837 | 204918 | |
| 13 | PRR20D | proline rich 20D | 227151 | 204918 | 234278 | |
| 13 | PRR20E | proline rich 20E | 234278 | 227151 | 118946 | |
| 13 | PCCA | propionyl-CoA carboxylase alpha subunit | 175198 | 043355 | 134864 | 10, 2, 17 |
| 13 | POMP | proteasome maturation protein | 132963 | 102755 | 139508 | 34, 7, 43 |
| 13 | PROZ | protein Z, vitamin K dependent plasma glycoprotein | 126231 | 126218 | 126226 | 1, 19, 17 |
| 13 | PCDH17 | protocadherin 17 | 118946 | 234278 | 139734 | 26, 11, 36 |
| 13 | PCDH20 | protocadherin 20 | 280165 | 083544 | 197991 | 15, 45, 39 |
| 13 | PCDH8 | protocadherin 8 | 136099 | 136110 | 102837 | 12, 8, 6 |
| 13 | PCDH9 | protocadherin 9 | 184226 | 197991 | 150361 | 11, 8, 16 |
| 13 | RAB20 | RAB20, member RAS oncogene family | 139832 | 134871 | 213995 | 16, 2, 17 |
| 13 | RAP2A | RAP2A, member of RAS oncogene family | 125249 | 139793 | 065150 | 18, 29, 22 |
| 13 | RASL11A | RAS like family 11 member A | 122035 | 122026 | 122034 | 13, 33, 14 |
| 13 | RASA3 | RAS p21 protein activator 3 | 185989 | 283199 | 283361 | 39, 1, 3 |
| 13 | RB1 | RB transcriptional corepressor 1 | 139687 | 136156 | 139679 | 18, 22, 42 |
| 13 | RCBTB1 | RCC1 and BTB domain containing protein 1 | 136144 | 136147 | 152213 | 31, 42, 29 |
| 13 | RCBTB2 | RCC1 and BTB domain containing protein 2 | 136161 | 139679 | 152207 | 11, 36, 2 |
| 13 | RGCC | regulator of cell cycle | 102760 | 172766 | 102763 | 20, 29, 5 |
| 13 | RFXAP | regulatory factor X associated protein | 133111 | 180440 | 120693 | 11, 22, 19 |
| 13 | RXFP2 | relaxin/insulin like family peptide receptor 2 | 133105 | 187676 | 073910 | |
| 13 | RFC3 | replication factor C subunit 3 | 133119 | 133121 | 172915 | 36, 45, 21 |
| 13 | ARHGEF7 | Rho guanine nucleotide exchange factor 7 | 102606 | 088448 | 153495 | 1, 22, 9 |
| 13 | RNASEH2B | ribonuclease H2 subunit B | 136104 | 186047 | 226792 | 11, 43, 29 |
| 13 | RPL21 | ribosomal protein L21 | 122026 | 152484 | 122035 | |
| 13 | RNF113B | ring finger protein 113B | 139797 | 152767 | 102572 | 24, 38, 8 |
| 13 | RNF17 | ring finger protein 17 | 132972 | 075673 | 151849 | 38, 32, 14 |
| 13 | RNF219 | ring finger protein 219 | 152193 | 152192 | 139746 | 22, 42, 24 |
| 13 | RNF6 | ring finger protein 6 | 127870 | 180730 | 132964 | 42, 39, 22 |
| 13 | RBM26 | RNA binding motif protein 26 | 139746 | 152193 | 102471 | 3, 29, 7 |
| 13 | POLR1D | RNA polymerase I subunit D | 186184 | 139517 | 169840 | 5, 4, 9 |
| 13 | RUBCNL | RUN and cysteine rich domain containing bedin 1 interacting | 102445 | 173988 | 136141 | 41, 39, 36 |
| | | protein like | | | | |
| 13 | SACS | sacsin molecular chaperone | 151835 | 102683 | 127863 | 45, 44, 1 |
| 13 | SGCG | sarcoglycan gamma | 102683 | 280175 | 151835 | 15, 25, 10 |
| 13 | SCEL | sciellin | 136155 | 005810 | 139737 | 24, 13, 1 |
| 13 | SERTM1 | serine rich and transmembrane domain containing 1 | 180440 | 133101 | 133111 | 14, 8, 44 |
| 13 | STK24 | serine/threonine kinase 24 | 102572 | 139797 | 088386 | 29, 3, 26 |
| 13 | SERPINE3 | serpin family E member 3 | 253309 | 150510 | 102786 | 22, 14, |
| 13 | SETDB2 | SET domain bifurcated 2 | 136169 | 102547 | 136147 | 22, 2, 15 |
| 13 | SUGT1 | SGT1 homolog, MIS12 kinetochore complex assembly cochaperone | 165416 | 139675 | 136110 | 10, 41, 28 |
| 13 | SHISA2 | shisa family member 2 | 180730 | 132932 | 127870 | 18, 17, 30 |
| 13 | SIAH3 | siah E3 ubiquitin protein ligase family member 3 | 215475 | 174015 | 123200 | 6, 8, |
| 13 | SAP18 | Sin3A associated protein 18 | 150459 | 150457 | 165480 | 30, 11, 29 |
| 13 | SLAIN1 | SLAIN motif family member 1 | 139737 | 136155 | 136160 | 8, 17, 38 |
| 13 | SLITRK1 | SLIT and NTRK like family member 1 | 178235 | 136158 | 184564 | 8, 38, 19 |
| 13 | SLITRK5 | SLIT and NTRK like family member 5 | 165300 | 184564 | 179399 | 11, 18, 15 |
| 13 | SLITRK6 | SLIT and NTRK like family member 6 | 184564 | 178235 | 165300 | 42, 24, 3 |
| 13 | SMAD9 | SMAD family member 9 | 120693 | 133111 | 120697 | 37, 31, 40 |
| 13 | SMIM2 | small integral membrane protein 2 | 139656 | 281883 | 151778 | |
| 13 | NALCN | sodium leak channel, non-selective | 102452 | 125247 | 198542 | 19, 8, 13 |
| 13 | SLC10A2 | solute carrier family 10 member 2 | 125255 | 134899 | 182346 | 17, 16, 14 |
| 13 | SLC15A1 | solute carrier family 15 member 1 | 088386 | 102572 | 088387 | 29, 35, 3 |
| 13 | SLC25A15 | solute carrier family 25 member 15 | 102743 | 102738 | 120690 | 1, 19, 43 |
| 13 | SLC25A30 | solute carrier family 25 member 30 | 174032 | 133112 | 136152 | 42, 13, 29 |
| 13 | SLC46A3 | solute carrier family 46 member 3 | 139508 | 132963 | 132938 | 20, 31, 24 |
| 13 | SLC7A1 | solute carrier family 7 member 1 | 139514 | 132938 | 122042 | 26, 16, 9 |
| 13 | SPG20 | spastic paraplegia 20 (Troyer syndrome) | 133104 | 242715 | 133101 | 45, 18, 31 |
| 13 | SPACA7 | sperm acrosome associated 7 | 153498 | 182968 | 126216 | 38, 32, |
| 13 | SPERT | spermatid associated | 174015 | 165837 | 215475 | 38, |
| 13 | SOHLH2 | spermatogenesis and oogenesis specific basic helix-loop-helix 2 | 120669 | 133083 | 250709 | 38, 3, 32 |
| 13 | SPATA13 | spermatogenesis associated 13 | 182957 | 273167 | 240654 | |
| 13 | SKA3 | spindle and kinetochore associated complex subunit 3 | 165480 | 150459 | 173141 | 3, 2, 41 |
| 13 | SPRY2 | sprouty RTK signaling antagonist 2 | 136158 | 102471 | 178235 | 26, 14, 37 |
| 13 | SPRYD7 | SPRY domain containing 7 | 123178 | 102753 | 204977 | 18, 3, 19 |
| 13 | SUPT20H | SPT20 homolog, SAGA complex component | 102710 | 120699 | 279238 | 38, 21, 42 |
| 13 | SOX1 | SRY-box 1 | 182968 | 153495 | 153498 | 20, 12, 8 |
| 13 | SOX21 | SRY-box21 | 125285 | 152749 | 125257 | 12, 8, 37 |
| 13 | STARD13 | StAR related lipid transfer domain containing 13 | 133121 | 133116 | 133119 | 43, 26, 29 |
| 13 | STOML3 | stomatin like 3 | 133115 | 150893 | 120685 | 42, 30, 24 |
| 13 | SERP2 | stress associated endoplasmic reticulum protein family member 2 | 151778 | 139656 | 102804 | 22, 20, 17 |
| 13 | SUCLA2 | succinate-CoA ligase ADP-forming beta subunit | 136143 | 102468 | 136159 | 2, 13, 8 |
| 13 | TBC1D4 | TBC1 domain family member 4 | 136111 | 118922 | 188243 | 22, 42, 25 |
| 13 | TGDS | TDP-glucose 4,6-dehydratase | 088451 | 080166 | 152749 | 43, 22, 4 |
| 13 | TEX26 | testis expressed 26 | 175664 | 102802 | 120694 | 10, 30, 38 |
| 13 | TEX29 | testis expressed 29 | 153495 | 102606 | 182968 | 6, 38, 37 |
| 13 | TEX30 | testis expressed 30 | 151287 | 175820 | 134901 | 4, 38, 34 |
| 13 | THSD1 | thrombospondin type 1 domain containing 1 | 136114 | 136098 | 136100 | 11, 25, 42 |
| 13 | TNFRSF19 | TNF receptor superfamily member 19 | 127863 | 151835 | 027001 | 42, 11, 2 |
| 13 | TFDP1 | transcription factor Dp-1 | 198176 | 150403 | 186009 | 4, 9, 37 |
| 13 | TRPC4 | transient receptor potential cation channel subfamily C member 4 | 133107 | 133110 | 120686 | 44, 10, 42 |
| 13 | TM9SF2 | transmembrane 9 superfamily member 2 | 125304 | 169508 | 125246 | 30, 2, 4 |
| 13 | TMC03 | transmembrane and coiled-coil domains 3 | 150403 | 150401 | 198176 | |
| 13 | TMTC4 | transmembrane and tetratricopeptide repeat containing 4 | 125247 | 134864 | 102452 | 24, 8, 13 |
| 13 | TPTE2 | transmembrane phosphoinositide 3-phosphatase and tensin homolog 2 | 132958 | 198033 | 196199 | 10, 37, 38 |
| 13 | TMEM255B | transmembrane protein 255B | 184497 | 185974 | 183087 | 1, 26, 25 |
| 13 | TRIM13 | tripartite motif containing 13 | 204977 | 123178 | 198553 | 22, 6, 12 |
| 13 | TPP2 | tripeptidyl peptidase 2 | 134900 | 102466 | 139780 | 38, 10, 11 |
| 13 | TSC22D1 | TSC22 domain family member 1 | 102804 | 151778 | 083635 | 29, 20, 25 |
| 13 | TUBA3C | tubulin alpha 3c | 198033 | 279081 | 132958 | 38, 5, 6 |
| 13 | TUBGCP3 | tubulin gamma complex associated protein 3 | 126216 | 153498 | 068650 | 40, 22, 38 |
| 13 | TDRD3 | tudor domain containing 3 | 083544 | 139734 | 280165 | 10, 18, 6 |
| 13 | TNFSF11 | tumor necrosis factor superfamily member 11 | 120659 | 023516 | 179813 | 22, 28, 10 |
| 13 | TNFSF13B | tumor necrosis factor superfamily member 13b | 102524 | 139826 | 283384 | 45, 5, 7 |
| 13 | TPT1 | tumor protein, translationally-controlled 1 | 133112 | 180332 | 174032 | 30, 43, 25 |
| 13 | UBAC2 | UBA domain containing 2 | 134882 | 088387 | 125245 | 35, 41, 21 |
| 13 | UBE2L5P | Ubiquitin conjugating enzyme E2 L5, pseudogene | 236444 | 102781 | 189403 | |
| 13 | UCHL3 | ubiquitin C-terminal hydrolase L3 | 118939 | 188243 | 136153 | 6, 31, 32 |
| 13 | UFM1 | ubiquitin fold modifier 1 | 120686 | 133107 | 150893 | 45, 11, 7 |
| 13 | UBL3 | ubiquitin like 3 | 122042 | 139514 | 102781 | 11, 8, 35 |
| 13 | USP12 | ubiquitin specific peptidase 12 | 152484 | 132975 | 122026 | 1, 22, 10 |
| 13 | USPL1 | ubiquitin specific peptidase like 1 | 132952 | 189403 | 132965 | 22, 7, 42 |
| 13 | UGGT2 | UDP-glucose glycoprotein glucosyltransferase 2 | 102595 | 102580 | 185352 | 29, 11, 6 |
| 13 | UPF3A | UPF3 regulator of nonsense transcripts homolog A (yeast) | 169062 | 130177 | 198824 | 33, 22, 29 |
| 13 | URAD | ureidoimidazoline (2-oxo-4-hydroxy-4-carboxy-5-) decarboxylase | 183463 | 165556 | 122025 | |
| 13 | UTP14C | UTP14, small subunit processome component homolog C (S. cerevisiae) | 253797 | 253710 | 197168 | |
| 13 | VPS36 | vacuolar protein sorting 36 homolog | 136100 | 136114 | 136108 | 26, 11, 22 |
| 13 | VWA8 | von Willebrand factor A domain containing 8 | 102763 | 102760 | 102780 | 29, 18, 11 |
| 13 | WASF3 | WAS protein family member 3 | 132970 | 132964 | 132975 | 29, 8, 14 |
| 13 | WDFY2 | WD repeat and FYVE domain containing 2 | 139668 | 102786 | 102796 | 13, 2, 39 |
| 13 | WBP4 | WW domain binding protein 4 | 120688 | 120690 | 165572 | 22, 43, 21 |
| 13 | ZIC2 | Zic family member 2 | 043355 | 139800 | 175198 | 11, 12, 26 |
| 13 | ZIC5 | Zic family member 5 | 139800 | 125246 | 043355 | 11, 12, 30 |
| 13 | ZC3H13 | zinc finger CCCH-type containing 13 | 123200 | 215475 | 080618 | 18, 5, 32 |
| 13 | ZDHHC20 | zinc finger DHHC-type containing 20 | 180776 | 173141 | 165487 | 1, 4, 32 |
| 13 | ZMYM2 | zinc finger MYM-type containing 2 | 121741 | 132950 | 121743 | 40, 11, 23 |
| 13 | ZMYM5 | zinc finger MYM-type containing 5 | 132950 | 121390 | 121741 | 4, 1, 2 |
| 13 | ZAR1L | zygote arrest 1-like | 189167 | 073910 | 139618 | 38, |
| 14 | ADCK1 | aarF domain containing kinase 1 | 063761 | 197734 | 021645 | 23, 27, 15 |
| 14 | ABHD12B | abhydrolase domain containing 12B | 131969 | 100504 | 100505 | 14, 2, 35 |
| 14 | ABHD4 | abhydrolase domain containing 4 | 100439 | 129562 | 255804 | 33, 13, 20 |
| 14 | ACTN1 | actinin alpha 1 | 072110 | 185650 | 139990 | 24, 12, 35 |
| 14 | ACTR10 | actin-related protein 10 homolog | 131966 | 139971 | 100567 | 29, 30, 5 |
| 14 | AHSA1 | activator of HSP90 ATPase activity 1 | 100591 | 151445 | 100593 | 18, 21, 1 |
| 14 | ACOT1 | acyl-CoA thioesterase 1 | 184227 | 170468 | 119673 | 29, 25, 35 |
| 14 | ACOT2 | acyl-CoA thioesterase 2 | 119673 | 184227 | 177465 | 29, 1, 6 |
| 14 | ACOT4 | acyl-CoA thioesterase 4 | 177465 | 119673 | 205669 | 1, 11, 6 |
| 14 | ACOT6 | acyl-CoA thioesterase 6 | 205669 | 177465 | 119661 | 10, 38, |
| 14 | ACYP1 | acylphosphatase 1 | 119640 | 119684 | 119703 | 29, 11, 31 |
| 14 | ADAM20 | ADAM metallopeptidase domain 20 | 134007 | 139985 | 133997 | 38, 8, |
| 14 | ADAM21 | ADAM metallopeptidase domain 21 | 139985 | 213463 | 134007 | 38, 6, 16 |
| 14 | AP1G2 | adaptor related protein complex 1 gamma 2 subunit | 213983 | 259431 | 092051 | |
| 14 | AP4S1 | adaptor related protein complex 4 sigma 1 subunit | 100478 | 196792 | 092148 | |
| 14 | AP5M1 | adaptor related protein complex 5 mu 1 subunit | 053770 | 070367 | 139977 | 24, 39, 42 |
| 14 | ADCY4 | adenylate cyclase 4 | 129467 | 213903 | 258973 | 15, 1, 32 |
| 14 | AK7 | adenylate kinase 7 | 140057 | 100744 | 090060 | 42, 23, 20 |
| 14 | ADSSL1 | adenylosuccinate synthase like 1 | 185100 | 203485 | 184990 | 1, 25, 9 |
| 14 | ARF6 | ADP ribosylation factor 6 | 165527 | 165525 | 100483 | 18, 24, 22 |
| 14 | AHNAK2 | AHNAK nucleoprotein 2 | 185567 | 166428 | 140104 | 43, 26, 6 |
| 14 | AJUBA | ajuba LIM protein | 129474 | 259132 | 100802 | 19, 9, 24 |
| 14 | AKAP5 | A-kinase anchoring protein 5 | 179841 | 089775 | 126804 | 36, 23, 22 |
| 14 | AKAP6 | A-kinase anchoring protein 6 | 151320 | 100852 | 151322 | 26, 8, 40 |
| 14 | AKT1 | AKT serine/threonine kinase 1 | 142208 | 184990 | 179627 | |
| 14 | ALDH6A1 | aldehyde dehydrogenase 6 family member A1 | 119711 | 119636 | 205659 | 17, 25, 8 |
| 14 | ALKBH1 | alkB homolog 1, histone H2A dioxygenase | 100601 | 100596 | 119705 | 14, 37, 2 |
| 14 | AMN | amnion associated transmembrane protein | 166126 | 131323 | 198752 | 17, 16, 28 |
| 14 | ANGEL1 | angel homolog 1 | 013523 | 071246 | 100565 | 38, 4, 36 |
| 14 | ANG | angiogenin | 214274 | 258818 | 259171 | 19, 3, 30 |
| 14 | ASB2 | ankyrin repeat and SOCS box containing 2 | 100628 | 140067 | 175699 | 15, 44, 25 |
| 14 | ANKRD9 | ankyrin repeat domain 9 | 156381 | 196663 | 089902 | 37, 9, 19 |
| 14 | APOPT1 | apoptogenic 1, mitochondrial | 256053 | 126214 | 126215 | 29, 27, 22 |
| 14 | AREL1 | apoptosis resistant E3 ubiquitin protein ligase 1 | 119682 | 119681 | 119616 | 42, 7, 22 |
| 14 | ACIN1 | apoptotic chromatin condensation inducer 1 | 100813 | 139880 | 179933 | 16, 12, 23 |
| 14 | APEX1 | apurinic/apyrimidinic endodeoxyribonuclease 1 | 100823 | 092094 | 165782 | 29, 9, 3 |
| 14 | ARG2 | arginase 2 | 081181 | 100564 | 100568 | 29, 40, 33 |
| 14 | ASPG | asparaginase | 166183 | 227729 | 066735 | 27, 17, 19 |
| 14 | ATXN3 | ataxin 3 | 066427 | 100815 | 279963 | 10, |
| 14 | ATL1 | atlastin GTPase 1 | 198513 | 012983 | 151748 | 8, 26, 17 |
| 14 | ABCD4 | ATP binding cassette subfamily D member 4 | 119688 | 119614 | 133980 | 1, 29, 36 |
| 14 | ATP5S | ATP synthase, H+ transporting, mitochondrial Fo | 125375 | 087299 | 100490 | 29, 26, 3 |
| | | complex subunit s (factor B) | | | | |
| 14 | ATP6V1D | ATPase H+ transporting V1 subunit D | 100554 | 072415 | 134001 | 43, 30, 31 |
| 14 | ARID4A | AT-rich interaction domain 4A | 032219 | 100567 | 196860 | 11, 26, 31 |
| 14 | ATG14 | autophagy related 14 | 126775 | 178974 | 182521 | 1, 11, 9 |
| 14 | ATG2B | autophagy related 2B | 066739 | 100739 | 100744 | 1, 39, 14 |
| 14 | BBOF1 | basal body orientation factor 1 | 119636 | 279461 | 119711 | 38, 34, 20 |
| 14 | BATF | basic leucine zipper ATF-like transcription factor | 156127 | 140044 | 119686 | 41, 22, 5 |
| 14 | BCL11B | B-cell CLL/lymphoma 11B | 127152 | 176605 | 183576 | 35, 39, 30 |
| 14 | BAG5 | BCL2 associated athanogene 5 | 166170 | 166166 | 126214 | 38, 1, 42 |
| 14 | BCL2L2 | BCL2 like 2 | 129473 | 235194 | 258643 | 29, 6, 14 |
| 14 | BCL2L2-PABPN1 | BCL2L2-PABPN1 readthrough | 258643 | 129473 | 100836 | |
| 14 | BMP4 | bone morphogenetic protein 4 | 125378 | 100523 | 100526 | 16, 37, 12 |
| 14 | BDKRB1 | bradykinin receptor B1 | 100739 | 258691 | 066739 | 9, 3, 10 |
| 14 | BDKRB2 | bradykinin receptor B2 | 168398 | 227051 | 258691 | |
| 14 | BEGAIN | brain enriched guanylate kinase associated | 183092 | 176473 | 185559 | 9, 28, 8 |
| 14 | BRMS1L | breast cancer metastasis-suppressor 1-like | 100916 | 174373 | 151332 | 26, 29, 25 |
| 14 | BRF1 | BRF1, RNA polymerase III transcription initiation factor subunit | 185024 | 183828 | 184887 | 13, 35, 26 |
| 14 | BAZ1A | bromodomain adjacent to zinc finger domain 1A | 198604 | 165410 | 100883 | 41, 6, 21 |
| 14 | BTBD6 | BTB domain containing 6 | 184887 | 185024 | 179364 | 30, 31, 25 |
| 14 | BTBD7 | BTB domain containing 7 | 011114 | 012963 | 133958 | 18, 11, 6 |
| 14 | CDH24 | cadherin 24 | 139880 | 222028 | 100813 | 9, 23, 1 |
| 14 | CLMN | calmin | 165959 | 100697 | 176438 | |
| 14 | CALM1 | calmodulin 1 | 198668 | 119720 | 165914 | 8, 31, 45 |
| 14 | CARMIL3 | capping protein regulator and myosin 1 linker 3 | 186648 | 187630 | 100884 | 18, 4, 14 |
| 14 | CTSG | cathepsin G | 100448 | 092009 | 100450 | 5, 15, 7 |
| 14 | CATSPERB | cation channel sperm associated auxiliary subunit beta | 133962 | 100796 | 165929 | 4, 27, 38 |
| 14 | CEBPE | CCAAT/enhancer binding protein epsilon | 092067 | 283654 | 092068 | 5, 6, 17 |
| 14 | CDC42BPB | CDC42 binding protein kinase beta | 198752 | 166126 | 283071 | 34, 14, 3 |
| 14 | CIDEB | cell death-inducing DFFA-like effector b | 136305 | 196943 | 213906 | 36, |
| 14 | CDCA4 | cell division cycle associated 4 | 170779 | 140104 | 183484 | 9, 7, 22 |
| 14 | CGRRF1 | cell growth regulator with ring finger domain 1 | 100532 | 197045 | 020577 | 31, 22, 1 |
| 14 | CEP128 | centrosomal protein 128 | 100629 | 211448 | 165409 | 36, |
| 14 | CEP170B | centrosomal protein 170B | 099814 | 179627 | 166428 | 26, 3, 37 |
| 14 | CBLN3 | cerebellin 3 precursor | 139899 | 205978 | 100441 | 36, 45, 6 |
| 14 | CHMP4A | charged multivesicular body protein 4A | 254505 | 139908 | 213920 | 21, 30, 26 |
| 14 | CHD8 | chromodomain helicase DNA binding protein 8 | 100888 | 092201 | 129472 | 3, 36, 31 |
| 14 | CHGA | chromogranin A | 100604 | 066455 | 100605 | 29, 31, 2 |
| 14 | C14orf1 | chromosome 14 open reading frame 1 | 133935 | 119685 | 119650 | 30, 29, 2 |
| 14 | C14orf105 | chromosome 14 open reading frame 105 | 100557 | 139977 | 151812 | 17, 4, 5 |
| 14 | C14orf119 | chromosome 14 open reading frame 119 | 179933 | 100813 | 283654 | |
| 14 | C14orf132 | Chromosome 14 open reading frame 132 | 227051 | 250366 | 168398 | 8, 31, 42 |
| 14 | C14orf159 | chromosome 14 open reading frame 159 | 133943 | 100784 | 119714 | 24, 13, 5 |
| 14 | C14orf166 | chromosome 14 open reading frame 166 | 087302 | 186469 | 087303 | 41, 40, 43 |
| 14 | C14orf169 | Chromosome 14 open reading frame 169 | 170468 | 187105 | 184227 | 9, 45, 6 |
| 14 | C14orf177 | chromosome 14 open reading frame 177 | 176605 | 100749 | 127152 | 15, 38, |
| 14 | C14orf178 | chromosome 14 open reading frame 178 | 197734 | 100603 | 063761 | |
| 14 | C14orf180 | chromosome 14 open reading frame 180 | 184601 | 258986 | 203485 | 15, 1, 25 |
| 14 | C14orf2 | chromosome 14 open reading frame 2 | 156411 | 088808 | 156414 | 31, 43, 34 |
| 14 | C14orf28 | chromosome 14 open reading frame 28 | 179476 | 189139 | 179454 | 15, 2, 38 |
| 14 | C14orf37 | chromosome 14 open reading frame 37 | 139971 | 151812 | 131966 | |
| 14 | C14orf39 | chromosome 14 open reading frame 39 | 179008 | 100614 | 184302 | 37, 38, 19 |
| 14 | C14orf79 | chromosome 14 open reading frame 79 | 140104 | 185567 | 170779 | 24, 6, 35 |
| 14 | C14orf80 | chromosome 14 open reading frame 80 | 185347 | 257341 | 184986 | 3, 9, 21 |
| 14 | C14orf93 | chromosome 14 open reading frame 93 | 100802 | 129474 | 100804 | 39, 9, 36 |
| 14 | CHURC1-FNTB | CHURC1-FNTB readthrough | 125954 | 258289 | 176153 | |
| 14 | CHURC1 | churchill domain containing 1 | 258289 | 279654 | 125954 | 1, 2, 25 |
| 14 | CMA1 | chymase 1 | 092009 | 100445 | 100448 | 23, |
| 14 | CMTM5 | CKLF like MARVEL transmembrane domain containing 5 | 166091 | 166090 | 197616 | 26, 8, 14 |
| 14 | CPSF2 | cleavage and polyadenylation specific factor 2 | 165934 | 183648 | 140090 | 18, 3, 42 |
| 14 | CIPC | CLOCK interacting pacemaker | 198894 | 119669 | 259164 | 31, 8, 42 |
| 14 | COCH | cochlin | 100473 | 092108 | 196792 | 11, 26, 31 |
| 14 | COQ6 | coenzyme Q6, monooxygenase | 119723 | 156050 | 187097 | 9, 13, 24 |
| 14 | CFL2 | cofilin 2 | 165410 | 129515 | 198604 | 25, 18, 15 |
| 14 | CCDC175 | coiled-coil domain containing 175 | 151838 | 050130 | 139970 | 44, 33, 20 |
| 14 | CCDC177 | coiled-coil domain containing 177 | 267909 | 175985 | 100647 | 14, 8, |
| 14 | CCDC85C | coiled-coil domain containing 85C | 205476 | 090061 | 182218 | 3, 44, 26 |
| 14 | CCDC88C | coiled-coil domain containing 88C | 015133 | 119714 | 100796 | 21, 9, 3 |
| 14 | CPNE6 | copine 6 | 100884 | 186648 | 129535 | 8, 31, 17 |
| 14 | CNIH1 | cornichon family AMPA receptor auxiliary protein 1 | 100528 | 100526 | 197045 | |
| 14 | COX16 | COX16, cytochrome c oxidase assembly homolog | 133983 | 100678 | 258644 | 10, 15, 30 |
| 14 | CKB | creatine kinase B | 166165 | 075413 | 166166 | 33, 14, 8 |
| 14 | CTAGE5 | CTAGE family member 5, ER export factor | 150527 | 258941 | 165355 | 19, 24, 2 |
| 14 | CLEC14A | C-type lectin domain family 14 member A | 176435 | 139874 | 100934 | 6, 32, 25 |
| 14 | CCNB1IP1 | cyclin B1 interacting protein 1 | 100814 | 136319 | 129484 | 3, 12, 6 |
| 14 | CINP | cyclin dependent kinase 2 interacting protein | 100865 | 022976 | 196663 | 29, 3, 18 |
| 14 | CDKN3 | cyclin dependent kinase inhibitor 3 | 100526 | 125378 | 100528 | 38, 43, 22 |
| 14 | CDKL1 | cyclin dependent kinase like 1 | 100490 | 125375 | 012983 | 9, 25, 24 |
| 14 | CCNK | cyclin K | 090061 | 183576 | 205476 | 16, 22, 23 |
| 14 | CRIP1 | cysteine rich protein 1 | 213145 | 182809 | 257341 | 9, 28, 7 |
| 14 | CRIP2 | cysteine rich protein 2 | 182809 | 182979 | 213145 | 27, 28, 26 |
| 14 | COX8C | cytochrome c oxidase subunit 8C | 187581 | 133958 | 175785 | 38, |
| 14 | CYP46A1 | cytochrome P450 family 46 subfamily A member 1 | 036530 | 182218 | 066629 | 26, 14, 28 |
| 14 | DCAF11 | DDB1 and CUL4 associated factor 11 | 100897 | 100889 | 259371 | 24, 39, 13 |
| 14 | DCAF4 | DDB1 and CUL4 associated factor 4 | 119599 | 205683 | 165861 | 13, 9, 27 |
| 14 | DCAF5 | DDB1 and CUL4 associated factor 5 | 139990 | 072110 | 081177 | 38, 1, 28 |
| 14 | DDHD1 | DDHD domain containing 1 | 100523 | 073712 | 125378 | 22, 26, 18 |
| 14 | DDX24 | DEAD-box helicase 24 | 089737 | 089723 | 165948 | 21, 24, 36 |
| 14 | DAD1 | defender against cell death 1 | 129562 | 211888 | 100439 | 31, 2, 11 |
| 14 | DHRS1 | dehydrogenase/reductase 1 | 157379 | 100949 | 196943 | 34, 36, 39 |
| 14 | DHRS2 | dehydrogenase/reductase 2 | 100867 | 092051 | 157326 | 4, 19, 3 |
| 14 | DHRS4 | dehydrogenase/reductase 4 | 157326 | 100867 | 187630 | 29, 1, 13 |
| 14 | DHRS4L2 | dehydrogenase/reductase 4 like 2 | 187630 | 157326 | 186648 | 3, 37, 13 |
| 14 | DHRS7 | dehydroaenase/reductase 7 | 100612 | 126773 | 100614 | 29, 33, 30 |
| 14 | DEGS2 | delta 4-desaturase, sphingolipid 2 | 168350 | 196405 | 100811 | 3, 37, 22 |
| 14 | DLK1 | delta like non-canonical Notch ligand 1 | 185559 | 183092 | 254656 | 32, 31, 2 |
| 14 | DICER1 | dicer 1, ribonuclease III | 100697 | 133937 | 165959 | 22, 43, 12 |
| 14 | DLST | dihydrolipoamide S-succinyltransferase | 119689 | 119608 | 198208 | 39, 40, 24 |
| 14 | DAAM1 | dishevelled associated activator of morphogenesis 1 | 100592 | 165617 | 181619 | 40, 36, 22 |
| 14 | DACT1 | dishevelled binding antagonist of beta catenin 1 | 165617 | 100578 | 100592 | 26, 12, 29 |
| 14 | DLGAP5 | DLG associated protein 5 | 126787 | 131981 | 178974 | 39, 5, 3 |
| 14 | POLE2 | DNA polymerase epsilon 2, accessory subunit | 100479 | 165506 | 197776 | 10, 4, 3 |
| 14 | DPF3 | double PHD fingers 3 | 205683 | 280188 | 119599 | 38, |
| 14 | DTD2 | D-tyrosyl-tRNA deacylase 2 (putative) | 129480 | 203546 | 214943 | 38, 23, 45 |
| 14 | DNAAF2 | dynein axonemal assembly factor 2 | 165506 | 168282 | 100479 | 34, 31, 10 |
| 14 | DNAL1 | dynein axonemal light chain 1 | 119661 | 205669 | 176903 | 1, 20, 26 |
| 14 | DYNC1H1 | dynein cytoplasmic 1 heavy chain 1 | 197102 | 078304 | 080824 | 26, 23, 43 |
| 14 | EAPP | E2F associated phosphoprotein | 129518 | 165389 | 129515 | 29, 45, 30 |
| 14 | EML1 | echinoderm microtubule associated protein like 1 | 066629 | 036530 | 196405 | 29, 8, 14 |
| 14 | EML5 | echinoderm microtubule associated protein like 5 | 165521 | 100722 | 165533 | 26, 31, 2 |
| 14 | ENTPD5 | ectonucleoside triphosphate diphosphohydrolase 5 | 187097 | 119723 | 279461 | 17, 26, 42 |
| 14 | EFCAB11 | EF-hand calcium binding domain 11 | 140025 | 053254 | 042088 | |
| 14 | EGLN3 | egl-9 family hypoxia inducible factor 3 | 129521 | 151322 | 165389 | 35, 32, 27 |
| 14 | ELMSAN1 | ELM2 and Myb/SANT domain containing 1 | 156030 | 176903 | 140043 | 4, 18, 24 |
| 14 | EFS | embryonal Fyn-associated substrate | 100842 | 283151 | 166090 | 11, 26, 15 |
| 14 | EVL | Enah/Vasp-like | 196405 | 066629 | 168350 | 39, 41, 36 |
| 14 | ERO1A | endoplasmic reticulum oxidoreductase 1 alpha | 197930 | 180998 | 100519 | |
| 14 | ERH | enhancer of rudimentary homolog (Drosophila) | 100632 | 100626 | 029364 | 29, 3, 28 |
| 14 | EDDM3A | epididymal protein 3A | 181562 | 259171 | 181552 | 33, 38, |
| 14 | EDDM3B | epididymal protein 3B | 181552 | 181562 | 169413 | 38, |
| 14 | EMC9 | ER membrane protein complex subunit 9 | 100908 | 092010 | 100911 | 29, 26, 41 |
| 14 | ESR2 | estrogen receptor 2 | 140009 | 054654 | 100714 | 38, 20, |
| 14 | ESRRB | estrogen related receptor beta | 119715 | 089916 | 071246 | 14, 25, 17 |
| 14 | EIF2S1 | eukaryotic translation initiation factor 2 subunit alpha | 134001 | 100554 | 100558 | 1, 30, 6 |
| 14 | EIF2B2 | eukaryotic translation initiation factor 2B subunit beta | 119718 | 119630 | 119684 | 29, 21, 41 |
| 14 | EIF5 | eukaryotic translation initiation factor 5 | 100664 | 185215 | 075413 | 29, 37, 22 |
| 14 | EXOC3L4 | exocyst complex component 3 like 4 | 205436 | 283071 | 185215 | 20, |
| 14 | EXOC5 | exocyst complex component 5 | 070367 | 165588 | 053770 | 7, 38, 31 |
| 14 | EXD2 | exonuclease 3′-5′ domain containing 2 | 081177 | 139990 | 100626 | 29, 38, 18 |
| 14 | FAM161B | family with sequence similarity 161 member B | 156050 | 119725 | 119723 | 8, 31, 38 |
| 14 | FAM177A1 | family with sequence similarity 177 member A1 | 151327 | 100883 | 092020 | |
| 14 | FAM179B | family with sequence similarity 179 member B | 198718 | 179454 | 185246 | 11, 29, 24 |
| 14 | FAM181A | family with sequence similarity 181 member A | 140067 | 175785 | 100628 | 38, 27, 8 |
| 14 | FAM71D | family with sequence similarity 71 member D | 172717 | 171723 | 072415 | 40, 38, 5 |
| 14 | FANCM | Fanconi anemia complementation group M | 187790 | 100442 | 129534 | 26, 38, 23 |
| 14 | FNTB | farnesyltransferase, CAAX box, beta | 257365 | 139998 | 125952 | 24, 35, 9 |
| 14 | FITM1 | fat storage inducing transmembrane protein 1 | 139914 | 259371 | 092010 | 25, 9, 31 |
| 14 | FBXO33 | F-box protein 33 | 165355 | 150527 | 165379 | 4, 11, 31 |
| 14 | FBXO34 | F-box protein 34 | 178974 | 126787 | 126775 | 24, 36, 42 |
| 14 | FCF1 | FCF1 rRNA-processing protein | 119616 | 119682 | 119596 | |
| 14 | FLVCR2 | feline leukemia virus subgroup C cellular receptor family member 2 | 119686 | 156127 | 119685 | 38, 32, 2 |
| 14 | FRMD6 | FERM domain containing 6 | 139926 | 139921 | 186469 | 45, 24, 18 |
| 14 | FERMT2 | fermitin family member 2 | 073712 | 100522 | 100523 | 1, 32, 18 |
| 14 | FLRT2 | fibronectin leucine rich transmembrane protein 2 | 185070 | 071537 | 054983 | |
| 14 | FSCB | fibrous sheath CABYR binding protein | 189139 | 165379 | 179476 | 38, 25, |
| 14 | FBLN5 | fibulin 5 | 140092 | 165929 | 100815 | 36, 1, 2 |
| 14 | FKBP3 | FK506 binding protein 3 | 100442 | 185246 | 187790 | 11, 6, 25 |
| 14 | FOXA1 | forkhead box A1 | 129514 | 151338 | 139865 | 33, 4, 26 |
| 14 | FOXG1 | forkhead box G1 | 176165 | 139910 | 184304 | |
| 14 | FOXN3 | forkhead box N3 | 053254 | 165533 | 140025 | 22, 7, 29 |
| 14 | FOS | Fos proto-oncogene, AP-1 transcription factor subunit | 170345 | 170348 | 140044 | 5, 42, 1 |
| 14 | FUT8 | fucosyltransferase 8 | 033170 | 125952 | 196553 | 23, 19, 13 |
| 14 | GNG2 | G protein subunit gamma 2 | 186469 | 139926 | 087302 | |
| 14 | GPR132 | G protein-coupled receptor 132 | 183484 | 170779 | 184916 | 39, 41, 26 |
| 14 | GPR135 | G protein-coupled receptor 135 | 181619 | 100592 | 126790 | 38, 9, 37 |
| 14 | GPR137C | G protein-coupled receptor 137C | 180998 | 087301 | 197930 | 11, 45, 22 |
| 14 | GPR33 | G protein-coupled receptor 33 (gene/pseudogene) | 214943 | 129480 | 151413 | |
| 14 | GPR65 | G protein-coupled receptor 65 | 140030 | 054983 | 100433 | 5, 1, 36 |
| 14 | GPR68 | G protein-coupled receptor 68 | 119714 | 133943 | 015133 | 32, 6, 10 |
| 14 | G2E3 | G2/M-phase specific E3 ubiquitin protein ligase | 092140 | 184304 | 092108 | 13, 31, 22 |
| 14 | GALC | galactosylceramidase | 054983 | 185070 | 140030 | 42, 31, 2 |
| 14 | LGALS3 | galectin 3 | 131981 | 168175 | 126787 | 35, 1, 34 |
| 14 | GEMIN2 | gem nuclear organelle associated protein 2 | 092208 | 100934 | 182400 | 22, 31, 34 |
| 14 | GTF2A1 | general transcription factor IIA subunit 1 | 165417 | 165409 | 140022 | 22, 23, 25 |
| 14 | GPHN | gephyrin | 171723 | 196553 | 172717 | 42, 24, 8 |
| 14 | GMFB | glia maturation factor beta | 197045 | 100528 | 100532 | |
| 14 | GNPNAT1 | glucosamine-phosphate N-acetyltransferase 1 | 100522 | 198252 | 073712 | 42, 25, 37 |
| 14 | GLRX5 | glutaredoxin 5 | 182512 | 176438 | 213231 | 11, 29, 14 |
| 14 | GPX2 | glutathione peroxidase 2 | 176153 | 125954 | 139998 | 4, 16, 37 |
| 14 | GSTZ1 | glutathione S-transferase zeta 1 | 100577 | 009830 | 100580 | 34, 21, 9 |
| 14 | PYGL | glycogen phosphorylase L | 100504 | 100503 | 131969 | 5, 4, 1 |
| 14 | GPHB5 | glycoprotein hormone beta 5 | 179600 | 126785 | 154001 | |
| 14 | GOLGA5 | golgin A5 | 066455 | 100600 | 100604 | 11, 45, 32 |
| 14 | GON7 | GON7, KEOPS complex subunit homolog | 170270 | 259066 | 012963 | 29, 4, 17 |
| 14 | GSC | goosecoid homeobox | 133937 | 196136 | 100697 | 6, 44, 10 |
| 14 | GPATCH2L | G-patch domain containing 2 like | 089916 | 119699 | 119715 | 5, |
| 14 | GZMB | granzyme B | 100453 | 100450 | 168952 | 5, 36, 37 |
| 14 | GZMH | granzyme H | 100450 | 100448 | 100453 | 30, 5, 10 |
| 14 | GSKIP | GSK3B interacting protein | 100744 | 066739 | 140057 | 29, 35, 4 |
| 14 | GCH1 | GTP cyclohydrolase 1 | 131979 | 020577 | 198554 | 22, 28, 19 |
| 14 | GMPR2 | guanosine monophosphate reductase 2 | 100938 | 129559 | 092330 | 29, 5, 32 |
| 14 | HAUS4 | HAUS augmin like complex subunit 4 | 092036 | 100462 | 259132 | 21, 5, 9 |
| 14 | HEATR4 | HEAT repeat containing 4 | 187105 | 133961 | 170468 | 38, 20, 14 |
| 14 | HEATR5A | HEAT repeat containing 5A | 129493 | 092148 | 203546 | 30, 41, 6 |
| 14 | HSP90AA1 | heat shock protein 90 alpha family class A member 1 | 080824 | 197102 | 140153 | 13, 30, 3 |
| 14 | HSPA2 | heat shock protein family A (Hsp70) member 2 | 126803 | 126804 | 165807 | 43, 38, 8 |
| 14 | HECTD1 | HECT domain E3 ubiquitin protein ligase 1 | 092148 | 100478 | 129493 | 40, 1, 42 |
| 14 | HNRNPC | heterogeneous nuclear ribonucleoprotein C (C1/C2) | 092199 | 169327 | 092200 | 21, 9, 34 |
| 14 | HHIPL1 | HHIP Iike 1 | 182218 | 205476 | 036530 | 1, 6, 14 |
| 14 | HOMEZ | homeobox and leucine zipper encoding | 215271 | 215277 | 235194 | 21, 38, 22 |
| 14 | HIF1A | hypoxia inducible factor 1 alpha subunit | 100644 | 258989 | 023608 | 42, 24, 45 |
| 14 | IGHA1 | Immunoglobulin heavy constant alpha 1 | 211895 | 211893 | 211896 | |
| 14 | IGHA2 | Immunoglobulin heavy constant alpha 2 (A2m marker) | 211890 | 184986 | 211891 | |
| 14 | IGHD | Immunoglobulin heavy constant delta | 211898 | 211897 | 211899 | |
| 14 | IGHE | Immunoglobulin heavy constant epsilon | 211891 | 211890 | 211892 | |
| 14 | IGHG1 | Immunoglobulin heavy constant gamma 1 (G1m marker) | 211896 | 211895 | 211897 | 36, 41, 10 |
| 14 | IGHG2 | Immunoglobulin heavy constant gamma 2 (G2m marker) | 211893 | 211892 | 211895 | |
| 14 | IGHG3 | Immunoglobulin heavy constant gamma 3 (G3m marker) | 211897 | 211896 | 211898 | |
| 14 | IGHG4 | Immunoglobulin heavy constant gamma 4 (G4m marker) | 211892 | 211891 | 211893 | |
| 14 | IGHM | Immunoglobulin heavy constant mu | 211899 | 211898 | 211900 | |
| 14 | IGHD1-1 | Immunoglobulin heavy diversity 1-1 | 236170 | 211931 | 211933 | |
| 14 | IGHD1-14 | Immunoglobulin heavy diversity 1-14 (non-functional) | 227108 | 211918 | 211920 | |
| 14 | IGHD1-20 | Immunoglobulin heavy diversity 1-20 | 237020 | 211912 | 211914 | |
| 14 | IGHD1-26 | Immunoglobulin heavy diversity 1-26 | 211907 | 236597 | 225825 | |
| 14 | IGHD1-7 | Immunoglobulin heavy diversity 1-7 | 237197 | 211925 | 228131 | |
| 14 | IGHD2-15 | Immunoglobulin heavy diversity 2-15 | 211918 | 211917 | 227108 | |
| 14 | IGHD2-2 | Immunoglobulin heavy diversity 2-2 | 211931 | 211930 | 236170 | |
| 14 | IGHD2-21 | Immunoglobulin heavy diversity 2-21 | 211912 | 211911 | 237020 | |
| 14 | IGHD2-8 | Immunoglobulin heavy diversity 2-8 | 211925 | 211924 | 237197 | |
| 14 | IGHD3-10 | Immunoglobulin heavy diversity 3-10 | 211923 | 232543 | 211924 | |
| 14 | IGHD3-16 | Immunoglobulin heavy diversity 3-16 | 211917 | 227800 | 211918 | |
| 14 | IGHD3-22 | Immunoglobulin heavy diversity 3-22 | 211911 | 227196 | 211912 | |
| 14 | IGHD3-3 | Immunoglobulin heavy diversity 3-3 | 211930 | 233655 | 211931 | |
| 14 | IGHD3-9 | Immunoglobulin heavy diversity 3-9 | 211924 | 211923 | 211925 | |
| 14 | IGHD4-11 | Immunoglobulin heavy diversity 4-11 (non-functional) | 232543 | 211921 | 211923 | |
| 14 | IGHD4-17 | Immunoglobulin heavy diversity 4-17 | 227800 | 211915 | 211917 | |
| 14 | IGHD4-23 | Immunoglobulin heavy diversity 4-23 (non-functional) | 227196 | 211909 | 211911 | |
| 14 | IGHD4-4 | Immunoglobulin heavy diversity 4-4 | 233655 | 211928 | 211930 | |
| 14 | IGHD5-12 | Immunoglobulin heavy diversity 5-12 | 211921 | 211920 | 232543 | |
| 14 | IGHD5-18 | Immunoglobulin heavy diversity 5-18 | 211915 | 211914 | 227800 | |
| 14 | IGHD5-24 | Immunoglobulin heavy diversity 5-24 (non-functional) | 211909 | 225825 | 227196 | |
| 14 | IGHD5-5 | Immunoglobulin heavy diversity 5-5 | 211928 | 228131 | 233655 | |
| 14 | IGHD6-13 | Immunoglobulin heavy diversity 6-13 | 211920 | 227108 | 211921 | |
| 14 | IGHD6-19 | Immunoglobulin heavy diversity 6-19 | 211914 | 237020 | 211915 | |
| 14 | IGHD6-25 | Immunoglobulin heavy diversity 6-25 | 225825 | 211907 | 211909 | |
| 14 | IGHD6-6 | Immunoglobulin heavy diversity 6-6 | 228131 | 237197 | 211928 | |
| 14 | IGHD7-27 | Immunoglobulin heavy diversity 7-27 | 236597 | 211905 | 211907 | |
| 14 | IGHJ1 | Immunoglobulin heavy joining 1 | 211905 | 211904 | 236597 | |
| 14 | IGHJ2 | Immunoglobulin heavy joining 2 | 211904 | 242887 | 211905 | |
| 14 | IGHJ3 | Immunoglobulin heavy joining 3 | 242887 | 240041 | 211904 | |
| 14 | IGHJ4 | Immunoglobulin heavy joining 4 | 240041 | 242472 | 242887 | |
| 14 | IGHJ5 | Immunoglobulin heavy joining 5 | 242472 | 211900 | 240041 | |
| 14 | IGHJ6 | Immunoglobulin heavy joining 6 | 211900 | 211899 | 242472 | |
| 14 | IGHV1-18 | Immunoglobulin heavy variable 1-18 | 211945 | 211944 | 211946 | |
| 14 | IGHV1-2 | Immunoglobulin heavy variable 1-2 | 211934 | 211933 | 211935 | |
| 14 | IGHV1-24 | Immunoglobulin heavy variable 1-24 | 211950 | 211949 | 211951 | |
| 14 | IGHV1-3 | Immunoglobulin heavy variable 1-3 | 211935 | 211934 | 276775 | |
| 14 | IGHV1-45 | Immunoglobulin heavy variable 1-45 | 211961 | 232216 | 211962 | |
| 14 | IGHV1-46 | Immunoglobulin heavy variable 1-46 | 211962 | 211961 | 211964 | |
| 14 | IGHV1-58 | Immunoglobulin heavy variable 1-58 | 211968 | 211967 | 224373 | |
| 14 | IGHV1-69 | Immunoglobulin heavy variable 1-69 | 211973 | 282359 | 211974 | 10, 36, 37 |
| 14 | IGHV1-69-2 | Immunoglobulin heavy variable 1-69-2 | 280411 | 281990 | 274576 | |
| 14 | IGHV2-26 | Immunoglobulin heavy variable 2-26 | 211951 | 211950 | 211952 | |
| 14 | IGHV2-5 | Immunoglobulin heavy variable 2-5 | 211937 | 282122 | 211938 | |
| 14 | IGHV2-70 | Immunoglobulin heavy variable 2-70 | 211974 | 211973 | 281990 | |
| 14 | IGHV2-70 | Immunoglobulin heavy variable 2-70 | 274576 | 280411 | 225698 | |
| 14 | IGHV3-11 | Immunoglobulin heavy variable 3-11 (gene/pseudogene) | 211941 | 282651 | 211942 | |
| 14 | IGHV3-13 | Immunoglobulin heavy variable 3-13 | 211942 | 211941 | 211943 | |
| 14 | IGHV3-15 | Immunoglobulin heavy variable 3-15 | 211943 | 211942 | 211944 | |
| 14 | IGHV3-16 | Immunoglobulin heavy variable 3-16 | 211944 | 211943 | 211945 | |
| 14 | IGHV3-20 | Immunoglobulin heavy variable 3-20 | 211946 | 211945 | 211947 | |
| 14 | IGHV3-21 | Immunoglobulin heavy variable 3-21 | 211947 | 211946 | 211949 | |
| 14 | IGHV3-23 | Immunoglobulin heavy variable 3-23 | 211949 | 211947 | 211950 | |
| 14 | IGHV3-30 | Immunoglobulin heavy variable 3-30 | 270550 | 211952 | 231475 | |
| 14 | IGHV3-33 | Immunoglobulin heavy variable 3-33 | 211955 | 231475 | 211956 | |
| 14 | IGHV3-35 | Immunoglobulin heavy variable 3-35 (non-functional) | 211957 | 211956 | 211958 | |
| 14 | IGHV3-38 | Immunoglobulin heavy variable 3-38 (non-functional) | 211958 | 211957 | 211959 | |
| 14 | IGHV3-43 | Immunoglobulin heavy variable 3-43 | 232216 | 211959 | 211961 | |
| 14 | IGHV3-48 | Immunoglobulin heavy variable 3-48 | 211964 | 211962 | 211965 | 10, 30, 36 |
| 14 | IGHV3-49 | Immunoglobulin heavy variable 3-49 | 211965 | 211964 | 211966 | |
| 14 | IGHV3-53 | Immunoglobulin heavy variable 3-53 | 211967 | 211966 | 211968 | |
| 14 | IGHV3-64 | Immunoglobulin heavy variable 3-64 | 223648 | 211970 | 211972 | |
| 14 | IGHV3-66 | Immunoglobulin heavy variable 3-66 | 211972 | 223648 | 282359 | |
| 14 | IGHV3-7 | Immunoglobulin heavy variable 3-7 | 211938 | 211937 | 282639 | |
| 14 | IGHV3-72 | Immunoglobulin heavy variable 3-72 | 225698 | 274576 | 211976 | |
| 14 | IGHV3-73 | Immunoglobulin heavy variable 3-73 | 211976 | 225698 | 224650 | |
| 14 | IGHV3-74 | Immunoglobulin heavy variable 3-74 | 224650 | 211976 | 211979 | |
| 14 | IGHV4-28 | Immunoglobulin heavy variable 4-28 | 211952 | 211951 | 270550 | |
| 14 | IGHV4-31 | Immunoglobulin heavy variable 4-31 | 231475 | 270550 | 211955 | |
| 14 | IGHV4-34 | Immunoglobulin heavy variable 4-34 | 211956 | 211955 | 211957 | |
| 14 | IGHV4-39 | Immunoglobulin heavy variable 4-39 | 211959 | 211958 | 232216 | |
| 14 | IGHV4-4 | Immunoglobulin heavy variable 4-4 | 276775 | 211935 | 282122 | |
| 14 | IGHV4-59 | Immunoglobulin heavy variable 4-59 | 224373 | 211968 | 211970 | |
| 14 | IGHV4-61 | Immunoglobulin heavy variable 4-61 | 211970 | 224373 | 223648 | |
| 14 | IGHV5-51 | Immunoglobulin heavy variable 5-51 | 211966 | 211965 | 211967 | |
| 14 | IGHV6-1 | Immunoglobulin heavy variable 6-1 | 211933 | 236170 | 211934 | |
| 14 | IGHV7-81 | Immunoglobulin heavy variable 7-81 (non-functional) | 211979 | 224650 | 080815 | |
| 14 | IPO4 | importin 4 | 196497 | 100918 | 259522 | 38, 42, 24 |
| 14 | ITPK1 | inositol-tetrakisphosphate 1-kinase | 100605 | 100604 | 165943 | 3, 35, 20 |
| 14 | INSM2 | INSM transcriptional repressor 2 | 168348 | 100906 | 174373 | 14, 8, |
| 14 | IFI27 | interferon alpha inducible protein 27 | 165949 | 165948 | 119632 | 13, 43, 16 |
| 14 | IFI27L1 | interferon alpha inducible protein 27 like 1 | 165948 | 089737 | 165949 | 3, 34, 14 |
| 14 | IFI27L2 | interferon alpha inducible protein 27 like 2 | 119632 | 165949 | 119698 | 31, 45, 13 |
| 14 | IRF2BPL | interferon regulatory factor 2 binding protein like | 119669 | 100565 | 198894 | 1, 27, 12 |
| 14 | IRF9 | interferon regulatory factor 9 | 213928 | 259529 | 100918 | 37, 39, 29 |
| 14 | IL25 | interleukin 25 | 166090 | 100842 | 166091 | |
| 14 | IFT43 | intraflagellar transport 43 | 119650 | 133935 | 119699 | 26, 1, 30 |
| 14 | INF2 | inverted formin, FH2 and WH2 domain containing | 203485 | 184601 | 185100 | 34, 1, 43 |
| 14 | DIO2 | iodothyronine deiodinase 2 | 211448 | 021645 | 100629 | |
| 14 | DIO3 | iodothyronine deiodinase 3 | 197406 | 254656 | 078304 | 32, 1, 43 |
| 14 | ISCA2 | iron-sulfur cluster assembly 2 | 165898 | 119655 | 119681 | 11, 44, 6 |
| 14 | ISM2 | isthmin 2 | 100593 | 100591 | 100596 | 32, 45, 44 |
| 14 | JAG2 | jagged 2 | 184916 | 183484 | 183828 | 1, 3, 26 |
| 14 | JKAMP | JNK1/MAPK8-associated membrane protein | 050130 | 126790 | 151838 | 30, 2, 7 |
| 14 | JDP2 | Jun dimerization protein 2 | 140044 | 170345 | 156127 | 21, 4, 40 |
| 14 | JPH4 | junctophilin 4 | 092051 | 213983 | 100867 | 31, 8, 14 |
| 14 | KLHDC1 | kelch domain containing 1 | 197776 | 100479 | 165516 | 31, 28, 8 |
| 14 | KLHDC2 | kelch domain containing 2 | 165516 | 197776 | 165525 | 38, 29, 16 |
| 14 | KLHL28 | kelch like family member 28 | 179454 | 179476 | 198718 | |
| 14 | KLHL33 | kelch like family member 33 | 185271 | 129566 | 092094 | 14, |
| 14 | KHNYN | KH and NYN domain containing | 100441 | 139899 | 100445 | 18, 1, 33 |
| 14 | KIAA0391 | KIAA0391 | 100890 | 092020 | 258790 | 21, 29, 31 |
| 14 | KIAA0586 | KIAA0586 | 100578 | 100575 | 165617 | 13, 38, 10 |
| 14 | KTN1 | kinectin 1 | 126777 | 182521 | 139946 | 10, 31, 4 |
| 14 | KIF26A | kinesin family member 26A | 066735 | 166183 | 258986 | 26, 3, 14 |
| 14 | KLC1 | kinesin light chain 1 | 126214 | 166170 | 256500 | 26, 8, 31 |
| 14 | L2HGDH | L-2-hydroxyglutarate dehydrogenase | 087299 | 100485 | 125375 | 26, 13, 17 |
| 14 | LTBP2 | latent transforming growth factor beta binding protein 2 | 119681 | 165898 | 119682 | 6, 26, 40 |
| 14 | LRP10 | LDL receptor related protein 10 | 197324 | 157227 | 139890 | 9, 20, 16 |
| 14 | LGMN | legumain | 100600 | 100599 | 066455 | 5, 32, 10 |
| 14 | LRFN5 | leucine rich repeat and fibronectin type III domain containing 5 | 165379 | 165355 | 189139 | 10, 8, 24 |
| 14 | LRRC74A | leucine rich repeat containing 74A | 100565 | 013523 | 119669 | 38, |
| 14 | LRRC9 | Leucine rich repeat containing 9 | 131951 | 139970 | 126773 | 12, 38, 25 |
| 14 | LRR1 | leucine rich repeat protein 1 | 165501 | 213741 | 165502 | 1, 9, 41 |
| 14 | LTB4R | leukotriene B4 receptor | 213903 | 213906 | 129467 | |
| 14 | LTB4R2 | leukotriene B4 receptor 2 | 213906 | 136305 | 213903 | 36, 7, 39 |
| 14 | LIN52 | lin-52 DREAM MuvB core complex component | 205659 | 119711 | 119614 | 39, 6, 5 |
| 14 | LINC00238 | Long intergenic non-protein coding RNA 238 | 196553 | 033170 | 171723 | 38, 19, 8 |
| 14 | LINC00521 | Long intergenic non-protein coding RNA 521 | 175699 | 100628 | 089723 | 38, |
| 14 | MDP1 | magnesium dependent phosphatase 1 | 213920 | 254505 | 255526 | 17, 15, 1 |
| 14 | MDGA2 | MAM domain containing glycosylphosphatidylinositol anchor 2 | 139915 | 165496 | 213741 | 45, 38, 6 |
| 14 | MGAT2 | mannosyl (alpha-1,6-)-glycoprotein | 168282 | 165502 | 165506 | 11, 45, 24 |
| | | beta-1,2-N-acetylglucosaminyltransferase | | | | |
| 14 | MBIP | MAP3K12 binding inhibitory protein 1 | 151332 | 100916 | 229415 | 7, 45, 4 |
| 14 | MMP14 | matrix metallopeptidase 14 | 157227 | 172590 | 197324 | 18, 13, 40 |
| 14 | MED6 | mediator complex subunit 6 | 133997 | 134007 | 133985 | 45, 42, 12 |
| 14 | MIA2 | melanoma inhibitory activity 2 | 150526 | 100941 | 258941 | 19, 17, 16 |
| 14 | MPP5 | membrane palmitoylated protein 5 | 072415 | 172717 | 100554 | |
| 14 | MTA1 | metastasis associated 1 | 182979 | 226174 | 182809 | 21, 3, 27 |
| 14 | MTHFD1 | methylenetetrahydrofolate dehydrogenase, cyclohydrolase and | 100714 | 140009 | 089775 | 43, 3, 1 |
| | | formyltetrahydrofolate synthetase 1 | | | | |
| 14 | METTL17 | methyltransferase like 17 | 165792 | 169385 | 165794 | 39, 41, 9 |
| 14 | METTL3 | methyltransferase like 3 | 165819 | 092203 | 165821 | 39, 36, 13 |
| 14 | MARK3 | microtubule affinity regulating kinase 3 | 075413 | 100664 | 166165 | 1, 3, 22 |
| 14 | MIPOL1 | mirror-image polydactyly 1 | 151338 | 183032 | 129514 | |
| 14 | MIS18BP1 | MIS18 binding protein 1 | 129534 | 187790 | 165496 | 22, 7, 12 |
| 14 | MRPL52 | mitochondrial ribosomal protein L52 | 172590 | 155465 | 157227 | 7, 43, 27 |
| 14 | MAPK1IP1L | mitogen-activated protein kinase 1 interacting protein 1 like | 168175 | 180008 | 131981 | 17, 22, 12 |
| 14 | MAP3K9 | mitogen-activated protein kinase kinase kinase 9 | 006432 | 133985 | 100731 | |
| 14 | MAP4K5 | mitogen-activated protein kinase kinase kinase kinase 5 | 012983 | 100490 | 198513 | 43, 44, 42 |
| 14 | MNAT1 | MNAT1, CDK activating kinase assembly factor | 020426 | 100625 | 126814 | 29, 6, 5 |
| 14 | MOAP1 | modulator of apoptosis 1 | 165943 | 100605 | 153485 | 8, 36, 17 |
| 14 | MOK | MOK protein kinase | 080823 | 140153 | 022976 | |
| 14 | MLH3 | mutL homolog 3 | 119684 | 119718 | 119640 | 41, 7, 30 |
| 14 | MAX | MYC associated factor X | 125952 | 257365 | 033170 | 18, 39, 16 |
| 14 | MYH6 | myosin heavy chain 6 | 197616 | 166091 | 092054 | 15, 24, 25 |
| 14 | MYH7 | myosin heavy chain 7 | 092054 | 197616 | 129460 | 25, 15, 18 |
| 14 | NAA30 | N(alpha)-acetyltransferase 30, NatC catalytic subunit | 139977 | 053770 | 100557 | 22, 1, 6 |
| 14 | NDUFB1 | NADH: ubiquinone oxidoreductase subunit B1 | 183648 | 279963 | 165934 | 31, 33, 10 |
| 14 | NOXRED1 | NADP dependent oxidoreductase domain containing 1 | 165555 | 100583 | 280308 | 38, 8, |
| 14 | NDRG2 | NDRG family member 2 | 165795 | 165794 | 179636 | 2, 8, 34 |
| 14 | NEDD8-MDP1 | NEDD8-MDP1 readthrough | 255526 | 213920 | 129559 | |
| 14 | NEDD8 | neural precursor cell expressed, developmentally down-regulated 8 | 129559 | 255526 | 100938 | 29, 31, 34 |
| 14 | NRL | neural retina leucine zipper | 129535 | 100884 | 100889 | 14, 5, 19 |
| 14 | NRXN3 | neurexin 3 | 021645 | 063761 | 211448 | 14, |
| 14 | NGB | neuroglobin | 165553 | 177108 | 009830 | 14, 8, 20 |
| 14 | NGDN | neuroguidin | 129460 | 092054 | 136367 | 3, 9, 30 |
| 14 | NPAS3 | neuronal PAS domain protein 3 | 151322 | 151320 | 129521 | |
| 14 | NFKBIA | NFKB inhibitor alpha | 100906 | 100902 | 168348 | 3, 41, 32 |
| 14 | NID2 | nidogen 2 | 087303 | 087302 | 168229 | 11, 1, 25 |
| 14 | NEK9 | NIMA related kinase 9 | 119638 | 119703 | 170348 | 29, 1, 22 |
| 14 | NIN | ninein | 100503 | 151748 | 100504 | 22, 1, 21 |
| 14 | NKX2-1 | NK2 homeobox 1 | 136352 | 229415 | 136327 | 31, 40, 20 |
| 14 | NKX2-8 | NK2 homeobox 8 | 136327 | 136352 | 198807 | 24, 20, 44 |
| 14 | NOP9 | NOP9 nucleolar protein | 196943 | 157379 | 136305 | 9, 24, 21 |
| 14 | NOVA1 | NOVA alternative splicing regulator 1 | 139910 | 281282 | 176165 | 11, 23, 1 |
| 14 | NPC2 | NPC intracellular cholesterol transporter 2 | 119655 | 183379 | 165898 | 43, 30, 29 |
| 14 | NRDE2 | NRDE-2, necessary for RNA interference, domain containing | 119720 | 100764 | 198668 | 13, 42, 24 |
| 14 | NEMF | nuclear export mediator factor | 165525 | 165516 | 165527 | 26, 7, 18 |
| 14 | NFATC4 | nuclear factor of activated T-cells 4 | 100968 | 129465 | 205978 | 42, 44, 6 |
| 14 | NUBPL | nucleotide binding protein like | 151413 | 214943 | 100852 | |
| 14 | NUDT14 | nudix hydrolase 14 | 183828 | 184916 | 185024 | 20, 41, 28 |
| 14 | NUMB | NUMB, endocytic adaptor protein | 133961 | 100767 | 187105 | 42, 9, 24 |
| 14 | NYNRIN | NYN domain and retroviral integrase containing | 205978 | 100968 | 139899 | 18, 27, 11 |
| 14 | OR10G2 | olfactory receptor family 10 subfamily G member 2 | 255582 | 255569 | 256553 | |
| 14 | OR10G3 | olfactory receptor family 10 subfamily G member 3 | 169208 | 165821 | 255569 | |
| 14 | OR11G2 | olfactory receptor family 11 subfamily G member 2 | 196832 | 184394 | 176219 | |
| 14 | OR11H12 | olfactory receptor family 11 subfamily H member 12 | 257115 | 080815 | 222036 | |
| 14 | OR11H2 | olfactory receptor family 11 subfamily H member 2 | 258453 | 187537 | 176294 | |
| 14 | OR11H4 | olfactory receptor family 11 subfamily H member 4 | 176198 | 258806 | 136319 | |
| 14 | OR11H6 | olfactory receptor family 11 subfamily H member 6 | 176219 | 196832 | 258806 | |
| 14 | OR11H7 | olfactory receptor family 11 subfamily H member 7 (gene/pseudogene) | 258806 | 176219 | 176198 | |
| 14 | OR4E2 | olfactory receptor family 4 subfamily E member 2 | 221977 | 256553 | 211776 | |
| 14 | OR4K1 | olfactory receptor family 4 subfamily K member 1 | 155249 | 176281 | 169488 | |
| 14 | OR4K13 | olfactory receptor family 4 subfamily K member 13 | 176253 | 169484 | 176246 | |
| 14 | OR4K14 | olfactory receptor family 4 subfamily K member 14 | 169484 | 196383 | 176253 | |
| 14 | OR4K15 | olfactory receptor family 4 subfamily K member 15 | 169488 | 155249 | 196383 | |
| 14 | OR4K17 | olfactory receptor family 4 subfamily K member 17 | 176230 | 176246 | 184394 | |
| 14 | OR4K2 | olfactory receptor family 4 subfamily K member 2 | 165762 | 176299 | 176281 | |
| 14 | OR4K5 | olfactory receptor family 4 subfamily K member 5 | 176281 | 165762 | 155249 | |
| 14 | OR4L1 | olfactory receptor family 4 subfamily L member 1 | 176246 | 176253 | 176230 | |
| 14 | OR4M1 | olfactory receptor family 4 subfamily M member 1 | 176299 | 182652 | 165762 | |
| 14 | OR4N2 | olfactory receptor family 4 subfamily N member 2 | 176294 | 258453 | 182652 | |
| 14 | OR4N5 | olfactory receptor family 4 subfamily N member 5 | 184394 | 176230 | 196832 | |
| 14 | OR4Q2 | olfactory receptor family 4 subfamily Q member 2 (gene/pseudogene) | 196383 | 169488 | 169484 | |
| 14 | OR4Q3 | olfactory receptor family 4 subfamily Q member 3 | 182652 | 176294 | 176299 | |
| 14 | OR5AU1 | olfactory receptor family 5 subfamily AU member 1 | 169327 | 232070 | 092199 | |
| 14 | OR6J1 | olfactory receptor family 6 subfamily J member 1 (gene/pseudogene) | 255804 | 100439 | 279510 | |
| 14 | OR6S1 | olfactory receptor family 6 subfamily S member 1 | 181803 | 258436 | 258818 | |
| 14 | OTX2 | orthodenticle homeobox 2 | 165588 | 070269 | 070367 | |
| 14 | OSGEP | O-sialoglycoprotein endopeptidase | 092094 | 185271 | 100823 | 25, 1, 13 |
| 14 | OTUB2 | OTU deubiquitinase, ubiquitin aldehyde binding 2 | 089723 | 175699 | 089737 | 38, 32, 14 |
| 14 | OXA1L | OXA1L, mitochondrial inner membrane protein | 155463 | 279510 | 155465 | 9, 21, 3 |
| 14 | PAX9 | paired box 9 | 198807 | 136327 | 183032 | 29, 24, 30 |
| 14 | PAPLN | papilin, proteoglycan like sulfated glycoprotein | 100767 | 080815 | 133961 | 17, 1, 24 |
| 14 | PNMA1 | paraneoplastic Ma antigen 1 | 176903 | 119661 | 156030 | 31, 28, 14 |
| 14 | PCNX1 | pecanex homolog 1 (Drosophila) | 100731 | 006432 | 197555 | 6, 25, 28 |
| 14 | PCNX4 | pecanex homolog 4 (Drosophila) | 126773 | 131951 | 100612 | 38, |
| 14 | PELI2 | pellino E3 ubiquitin protein ligase family member 2 | 139946 | 126777 | 070269 | 26, 39, 11 |
| 14 | PIGH | phosphatidylinositol glycan anchor biosynthesis class H | 100564 | 054690 | 081181 | 45, 13, 17 |
| 14 | PCK2 | phosphoenolpyruvate carboxykinase 2, mitochondrial | 100889 | 129535 | 100897 | 3, 21, 17 |
| 14 | PACS2 | phosphofurin acidic cluster sorting protein 2 | 179364 | 184887 | 226174 | 38, 34, 18 |
| 14 | PLD4 | phospholipase D family member 4 | 166428 | 099814 | 185567 | 41, 7, 36 |
| 14 | PNN | pinin, desmosome associated protein | 100941 | 182400 | 150526 | 39, 24, 36 |
| 14 | PGF | placental growth factor | 119630 | 198208 | 119718 | 32, 4, 25 |
| 14 | PLEK2 | pleckstrin 2 | 100558 | 134001 | 198133 | 2, 3, 35 |
| 14 | PLEKHD1 | pleckstrin homology and coiled-coil domain containing D1 | 175985 | 029364 | 267909 | 40, 23, |
| 14 | PLEKHG3 | pleckstrin homology and RhoGEF domain containing G3 | 126822 | 165807 | 070182 | 9, 1, 37 |
| 14 | PLEKHH1 | pleckstrin homology, MyTH4 and FERM domain containing H1 | 054690 | 198133 | 100564 | 8, 4, 29 |
| 14 | PABPN1 | poly(A) binding protein nuclear 1 | 100836 | 258643 | 092096 | 3, 1, 26 |
| 14 | PAPOLA | poly(A) polymerase alpha | 090060 | 140057 | 100749 | 42, 11, 40 |
| 14 | PARP2 | poly(ADP-ribose) polymerase 2 | 129484 | 100814 | 129566 | 18, 33, 40 |
| 14 | GALNT16 | polypeptide N-acetylgalactosaminyltransferase 16 | 100626 | 081177 | 100632 | 8, |
| 14 | KCNK10 | potassium two pore domain channel subfamily K member 10 | 100433 | 140030 | 042317 | 30, 8, 17 |
| 14 | KCNK13 | potassium two pore domain channel subfamily K member 13 | 152315 | 042088 | 100764 | 26, 5, 38 |
| 14 | KCNH5 | potassium voltage-gated channel subfamily H member 5 | 140015 | 139973 | 126785 | 26, 35, 38 |
| 14 | POTEG | POTE ankyrin domain family member G | 187537 | 279508 | 258453 | 33, 24, 10 |
| 14 | POTEM | POTE ankyrin domain family member M | 222036 | 257115 | 279508 | 33, 38, |
| 14 | PRPF39 | pre-mRNA processing factor 39 | 185246 | 198718 | 100442 | 13, 40, 18 |
| 14 | PSEN1 | presenilin 1 | 080815 | 119707 | 100767 | 24, 16, 42 |
| 14 | PRIMA1 | proline rich membrane anchor 1 | 175785 | 187581 | 140067 | 26, 1, 31 |
| 14 | PROX2 | prospero homeobox 2 | 119608 | 119596 | 119689 | 23, 44, |
| 14 | PTGDR | prostaglandin D2 receptor | 168229 | 087303 | 125384 | 16, 44, 19 |
| 14 | PTGER2 | prostaglandin E receptor 2 | 125384 | 168229 | 087301 | 1, 7, 17 |
| 14 | PTGR2 | prostaglandin reductase 2 | 140043 | 156030 | 258653 | 40, 25, 23 |
| 14 | PSMC1 | proteasome 26S subunit, ATPase 1 | 100764 | 152315 | 119720 | 3, 27, 11 |
| 14 | PSMC6 | proteasome 26S subunit, ATPase 6 | 100519 | 197930 | 198252 | 4, 34, 44 |
| 14 | PSME1 | proteasome activator subunit 1 | 092010 | 139914 | 100908 | 30, 7, 5 |
| 14 | PSME2 | proteasome activator subunit 2 | 100911 | 100908 | 092098 | 38, |
| 14 | PSMA3 | proteasome subunit alpha 3 | 100567 | 131966 | 032219 | 4, 37, 6 |
| 14 | PSMA6 | proteasome subunit alpha 6 | 100902 | 258790 | 100906 | 29, 4, 3 |
| 14 | PSMB11 | proteasome subunit beta 11 | 222028 | 100804 | 139880 | 39, 38, |
| 14 | PSMB5 | proteasome subunit beta 5 | 100804 | 100802 | 222028 | 30, 34, 9 |
| 14 | PRMT5 | protein arginine methyltransferase 5 | 100462 | 100461 | 092036 | 3, 13, 45 |
| 14 | PRKCH | protein kinase C eta | 027075 | 139974 | 182107 | |
| 14 | PRKD1 | protein kinase D1 | 184304 | 176165 | 092140 | |
| 14 | POMT2 | protein O-mannosyltransferase 2 | 009830 | 165553 | 100577 | 6, 1, 20 |
| 14 | PPP1R13B | protein phosphatase 1 regulatory subunit 13B | 088808 | 100711 | 156411 | 40, 38, 16 |
| 14 | PPP1R36 | protein phosphatase 1 regulatory subunit 36 | 165807 | 126803 | 126822 | 28, 3, 38 |
| 14 | PPP1R3E | protein phosphatase 1 regulatory subunit 3E | 235194 | 215271 | 129473 | 26, 14, 29 |
| 14 | PPP2R5E | protein phosphatase 2 regulatory subunit B′epsilon | 154001 | 179600 | 140006 | 4, 11, 36 |
| 14 | PPP2R5C | protein phosphatase 2 regulatory subunit B′gamma | 078304 | 197406 | 197102 | |
| 14 | PPP2R3C | protein phosphatase 2 regulatory subunit B″gamma | 092020 | 151327 | 100890 | 10, 30, 38 |
| 14 | PPP4R3A | protein phosphatase 4 regulatory subunit 3A | 100796 | 015133 | 133962 | 7, 2, 18 |
| 14 | PPP4R4 | protein phosphatase 4 regulatory subunit 4 | 119698 | 119632 | 140093 | 42, 23, 14 |
| 14 | PPM1A | protein phosphatase, Mg2+/Mn2+ dependent 1A | 100614 | 100612 | 179008 | 26, 1, 21 |
| 14 | PTPN21 | protein tyrosine phosphatase, non-receptor type 21 | 070778 | 042317 | 100722 | 11, 31, 29 |
| 14 | PNP | purine nucleoside phosphorylase | 198805 | 165782 | 182545 | 3, 30, 29 |
| 14 | RABGGTA | Rab geranylgeranyltransferase alpha subunit | 100949 | 092295 | 157379 | 36, 2, 31 |
| 14 | RAB15 | RAB15, member RAS oncogene family | 139998 | 176153 | 257365 | 4, 26, 31 |
| 14 | RAB2B | RAB2B, member RAS oncogene family | 129472 | 100888 | 092203 | 1, 26, 31 |
| 14 | RAD51B | RAD51 paralog B | 182185 | 072121 | 185650 | 10, 5, 6 |
| 14 | RALGAPA1 | Ral GTPase activating protein catalytic alpha subunit 1 | 174373 | 168348 | 100916 | |
| 14 | RIN3 | Ras and Rab interactor 3 | 100599 | 140090 | 100600 | 36, 7, 5 |
| 14 | RHOJ | ras homolog family member J | 126785 | 140015 | 179600 | 45, 11, 43 |
| 14 | REC8 | REC8 meiotic recombination protein | 100918 | 213928 | 196497 | 39, 38, 36 |
| 14 | RIPK3 | receptor interacting serine/threonine kinase 3 | 129465 | 258973 | 100968 | 36, 4, 5 |
| 14 | RGS6 | regulator of G-protein signaling 6 | 182732 | 197555 | 280188 | 2, 38, |
| 14 | RCOR1 | REST corepressor 1 | 089902 | 156381 | 131323 | 22, 29, 39 |
| 14 | RTN1 | reticulon 1 | 139970 | 151838 | 131951 | 8, 26, 14 |
| 14 | RD3L | retinal degeneration 3-like | 227729 | 156414 | 166183 | 29, 14, |
| 14 | RPGRIP1 | retinitis pigmentosa GTPase regulator interacting protein 1 | 092200 | 092199 | 092201 | 38, 25, 14 |
| 14 | RDH11 | retinol dehydrogenase 11 (all-trans/9-cis/11-cis) | 072042 | 258466 | 139988 | 33, 2, 7 |
| 14 | RDH12 | retinol dehydrogenase 12 (all-trans/9-cis/11-cis) | 139988 | 072042 | 072121 | |
| 14 | RTL1 | retrotransposon-like 1 | 254656 | 185559 | 197406 | 25, 10, 32 |
| 14 | ARHGAP5 | Rho GTPase activating protein 5 | 100852 | 151413 | 151320 | 4, 40, 23 |
| 14 | ARHGEF40 | Rho guanine nucleotide exchange factor 40 | 165801 | 173431 | 165804 | 27, 40, 32 |
| 14 | RNASE1 | ribonuclease A family member 1, pancreatic | 129538 | 169413 | 169397 | 30, 1, 2 |
| 14 | RNASE10 | ribonuclease A family member 10 (inactive) | 182545 | 198805 | 188655 | 44, |
| 14 | RNASE11 | ribonuclease A family member 11 (inactive) | 173464 | 259060 | 258436 | 38, 32, 8 |
| 14 | RNASE12 | ribonuclease A family member 12 (inactive) | 258436 | 173464 | 181803 | 38, |
| 14 | RNASE13 | ribonuclease A family member 13 (inactive) | 206150 | 179636 | 165799 | 38, |
| 14 | RNASE2 | ribonuclease A family member 2 | 169385 | 169397 | 165792 | 5, 7, 36 |
| 14 | RNASE3 | ribonuclease A family member 3 | 169397 | 129538 | 169385 | 5, 18, 7 |
| 14 | RNASE4 | ribonuclease A family member 4 | 258818 | 181803 | 214274 | |
| 14 | RNASE7 | ribonuclease A family member 7 | 165799 | 206150 | 173431 | 15, 39, 27 |
| 14 | RNASE8 | ribonuclease A family member 8 | 173431 | 165799 | 165801 | |
| 14 | RNASE9 | ribonuclease A family member 9 (inactive) | 188655 | 182545 | 259060 | |
| 14 | RNASE6 | ribonuclease A family member k6 | 169413 | 181552 | 129538 | 30, 2, 45 |
| 14 | RPL10L | ribosomal protein L10 like | 165496 | 129534 | 139915 | 38, 8, |
| 14 | RPL36AL | ribosomal protein L36a like | 165502 | 165501 | 168282 | |
| 14 | RPS29 | ribosomal protein S29 | 213741 | 139915 | 165501 | 12, 7, 33 |
| 14 | RPS6KA5 | ribosomal protein S6 kinase A5 | 100784 | 165914 | 133943 | 11, 41, 32 |
| 14 | RPS6KL1 | ribosomal protein S6 kinase like 1 | 198208 | 119689 | 119630 | 18, 8, 38 |
| 14 | RNF212B | ring finger protein 212B | 215277 | 092068 | 215271 | |
| 14 | RNF31 | ring finger protein 31 | 092098 | 100911 | 259529 | |
| 14 | RBM23 | RNA binding motif protein 23 | 100461 | 139890 | 100462 | 24, 34, 32 |
| 14 | RBM25 | RNA binding motif protein 25 | 119707 | 165861 | 080815 | 11, 43, 5 |
| 14 | REM2 | RRAD and GEM like GTPase 2 | 139890 | 197324 | 100461 | 31, 36, 20 |
| 14 | SAV1 | salvador family WW domain containing protein 1 | 151748 | 198513 | 100503 | 45, 11, 38 |
| 14 | SCFD1 | sec1 family domain containing 1 | 092108 | 092140 | 100473 | 10, 31, 45 |
| 14 | SEC23A | Sec23 homolog A, coat complex II component | 100934 | 176435 | 092208 | 42, 24, 45 |
| 14 | SEL1L | SEL1L ERAD E3 ligase adaptor subunit | 071537 | 140022 | 185070 | 11, 22, 45 |
| 14 | SRSF5 | serine and arginine rich splicing factor 5 | 100650 | 100647 | 100652 | |
| 14 | SPTLC2 | serine palmitoyltransferase long chain base subunit 2 | 100596 | 100593 | 100601 | 18, 37, 22 |
| 14 | SPTSSA | serine palmitoyltransferase small subunit A | 165389 | 129521 | 129518 | 7, 34, 4 |
| 14 | STYX | serine/threonine/tyrosine interacting protein | 198252 | 100519 | 100522 | 22, 43, 7 |
| 14 | SERPINA1 | serpin family A member 1 | 197249 | 258597 | 186910 | 19, 36, 20 |
| 14 | SERPINA10 | serpin family A member 10 | 140093 | 119698 | 170099 | 19, 15, |
| 14 | SERPINA11 | serpin family A member 11 | 186910 | 197249 | 170054 | 19, 44, 12 |
| 14 | SERPINA12 | serpin family A member 12 | 165953 | 170054 | 100665 | 35, 24, 19 |
| 14 | SERPINA2 | serpin family A member 2 (gene/pseudogene) | 258597 | 170099 | 197249 | |
| 14 | SERPINA3 | serpin family A member 3 | 196136 | 273259 | 133937 | |
| 14 | SERPINA3 | serpin family A member 3 | 273259 | 188488 | 196136 | |
| 14 | SERPINA4 | serpin family A member 4 | 100665 | 165953 | 188488 | 2, 38, 19 |
| 14 | SERPINA5 | serpin family A member 5 | 188488 | 100665 | 273259 | 2, 38, 19 |
| 14 | SERPINA6 | serpin family A member 6 | 170099 | 140093 | 258597 | 19, 23, 28 |
| 14 | SERPINA9 | serpin family A member 9 | 170054 | 186910 | 165953 | 41, 22, 21 |
| 14 | SETD3 | SET domain containing 3 | 183576 | 127152 | 090061 | 31, 1, 43 |
| 14 | SDR39U1 | short chain dehydrogenase/reductase family 39U member 1 | 100445 | 100441 | 092009 | 9, 26, 5 |
| 14 | SIPA1L1 | signal induced proliferation associated 1 like 1 | 197555 | 100731 | 182732 | 43, 36, 31 |
| 14 | SRP54 | signal recognition particle 54 | 100883 | 198604 | 151327 | 4, 5, 13 |
| 14 | SIVA1 | SIVA1 apoptosis inducing factor | 184990 | 185100 | 142208 | 41, 39, 34 |
| 14 | SIX1 | SIX homeobox 1 | 126778 | 184302 | 100625 | 29, |
| 14 | SIX4 | SIX homeobox 4 | 100625 | 126778 | 020426 | |
| 14 | SIX6 | SIX homeobox 6 | 184302 | 179008 | 126778 | 31, 5, 14 |
| 14 | SNAPC1 | small nuclear RNA activating complex polypeptide 1 | 023608 | 100644 | 139973 | 18, 40, 7 |
| 14 | SNW1 | SNW domain containing 1 | 100603 | 119705 | 197734 | 13, 7, 21 |
| 14 | SLC10A1 | solute carrier family 10 member 1 | 100652 | 100650 | 198732 | 19, 18, 30 |
| 14 | SLC22A17 | solute carrier family 22 member 17 | 092096 | 100836 | 283151 | 26, 31, 8 |
| 14 | SLC22A17 | solute carrier family 22 member 17 | 283151 | 092096 | 100842 | 26, 31, 8 |
| 14 | SLC24A4 | solute carrier family 24 member 4 | 140090 | 165934 | 100599 | 25, 5, 8 |
| 14 | SLC25A21 | solute carrier family 25 member 21 | 183032 | 198807 | 151338 | 29, 34, 30 |
| 14 | SLC25A29 | solute carrier family 25 member 29 | 197119 | 100811 | 140107 | |
| 14 | SLC25A47 | solute carrier family 25 member 47 | 140107 | 197119 | 140105 | 19, 23, 5 |
| 14 | SLC35F4 | solute carrier family 35 member F4 | 151812 | 100557 | 139971 | |
| 14 | SLC38A6 | solute carrier family 38 member 6 | 139974 | 126814 | 027075 | 13, 45, 9 |
| 14 | SLC39A2 | solute carrier family 39 member 2 | 165794 | 165792 | 165795 | 33, 30, 44 |
| 14 | SLC39A9 | solute carrier family 39 member 9 | 029364 | 100632 | 175985 | 13, 42, 2 |
| 14 | SLC7A7 | solute carrier family 7 member 7 | 155465 | 155463 | 172590 | 36, 43, 29 |
| 14 | SLC7A8 | solute carrier family 7 member 8 | 092068 | 092067 | 215277 | 18, 29, 24 |
| 14 | SLC8A3 | solute carrier family 8 member A3 | 100678 | 198732 | 133983 | 11, 14, 8 |
| 14 | SSTR1 | somatostatin receptor 1 | 139874 | 139865 | 176435 | 9, 28, 8 |
| 14 | SNX6 | sorting nexin 6 | 129515 | 129518 | 165410 | 29, 13, 32 |
| 14 | SOS2 | SOS Ras/Rho guanine nucleotide exchange factor 2 | 100485 | 100483 | 087299 | 22, 26, 7 |
| 14 | SALL2 | spalt like transcription factor 2 | 165821 | 165819 | 169208 | |
| 14 | SMOC1 | SPARC related modular calcium binding 1 | 198732 | 100652 | 100678 | 9, 8, 26 |
| 14 | SPTB | spectrin beta, erythrocytic | 070182 | 126822 | 279654 | 26, 18, 25 |
| 14 | SYNE3 | spectrin repeat containing nuclear envelope family member 3 | 176438 | 165959 | 182512 | 26, 18, 40 |
| 14 | SYNE2 | spectrin repeat containing nuclear envelope protein 2 | 054654 | 126821 | 140009 | 18, 24, 40 |
| 14 | SPATA7 | spermatogenesis associated 7 | 042317 | 100433 | 070778 | 31, 38, 8 |
| 14 | SGPP1 | sphingosine-1-phosphate phosphatase 1 | 126821 | 140006 | 054654 | 4, 11, 32 |
| 14 | SUPT16H | SPT16 homolog, facilitates chromatin remodeling subunit | 092201 | 092200 | 100888 | 21, 30, 13 |
| 14 | SLIRP | SRA stem-loop interacting RNA binding protein | 119705 | 100601 | 100603 | 25, 45, 37 |
| 14 | SAMD15 | sterile alpha motif domain containing 15 | 100583 | 100580 | 165555 | 30, 38, 36 |
| 14 | SAMD4A | sterile alpha motif domain containing 4A | 020577 | 100532 | 131979 | |
| 14 | STON2 | stonin 2 | 140022 | 165417 | 071537 | 18, 4, 7 |
| 14 | STRN3 | striatin 3 | 196792 | 100473 | 100478 | 11, 18, 31 |
| 14 | SOCS4 | suppressor of cytokine signaling 4 | 180008 | 198554 | 168175 | 13, 4, 43 |
| 14 | SFTA3 | surfactant associated 3 | 229415 | 151332 | 136352 | 40, 20, 10 |
| 14 | SUSD6 | sushi domain containing 6 | 100647 | 267909 | 100650 | 29, 22, 26 |
| 14 | SYNDIG1L | synapse differentiation inducing 1 like | 183379 | 133980 | 119655 | 26, 28, 8 |
| 14 | SYNJ2BP | synaptojanin 2 binding protein | 213463 | 258644 | 139985 | 29, 9, 4 |
| 14 | SYT16 | synaptotagmin 16 | 139973 | 023608 | 140015 | 45, 33, 38 |
| 14 | SYNJ2BP- | SYNJ2BP-COX16 readthrough | 258644 | 133983 | 213463 | |
| | COX16 | | | | | |
| 14 | STXBP6 | syntaxin binding protein 6 | 168952 | 100453 | 281282 | 44, 21, 35 |
| 14 | TRAJ1 | T cell receptor alpha joining 1 (non-functional) | 211888 | 211887 | 129562 | |
| 14 | TRAJ10 | T cell receptor alpha joining 10 | 211879 | 211878 | 211880 | |
| 14 | TRAJ11 | T cell receptor alpha joining 11 | 211878 | 211877 | 211879 | |
| 14 | TRAJ12 | T cell receptor alpha joining 12 | 211877 | 211876 | 211878 | |
| 14 | TRAJ13 | T cell receptor alpha joining 13 | 211876 | 211875 | 211877 | |
| 14 | TRAJ14 | T cell receptor alpha joining 14 | 211875 | 211873 | 211876 | |
| 14 | TRAJ16 | T cell receptor alpha joining 16 | 211873 | 211872 | 211875 | |
| 14 | TRAJ17 | T cell receptor alpha joining 17 | 211872 | 211871 | 211873 | |
| 14 | TRAJ18 | T cell receptor alpha joining 18 | 211871 | 211870 | 211872 | |
| 14 | TRAJ19 | T cell receptor alpha joining 19 (non-functional) | 211870 | 211869 | 211871 | |
| 14 | TRAJ2 | T cell receptor alpha joining 2 (non-functional) | 211887 | 211886 | 211888 | |
| 14 | TRAJ20 | T cell receptor alpha joining 20 | 211869 | 211868 | 211870 | |
| 14 | TRAJ21 | T cell receptor alpha joining 21 | 211868 | 211867 | 211869 | |
| 14 | TRAJ22 | T cell receptor alpha joining 22 | 211867 | 211866 | 211868 | |
| 14 | TRAJ23 | T cell receptor alpha joining 23 | 211866 | 211865 | 211867 | |
| 14 | TRAJ24 | T cell receptor alpha joining 24 | 211865 | 211864 | 211866 | |
| 14 | TRAJ25 | T cell receptor alpha joining 25 (non-functional) | 211864 | 211863 | 211865 | |
| 14 | TRAJ26 | T cell receptor alpha joining 26 | 211863 | 211862 | 211864 | |
| 14 | TRAJ27 | T cell receptor alpha joining 27 | 211862 | 211861 | 211863 | |
| 14 | TRAJ28 | T cell receptor alpha joining 28 | 211861 | 211860 | 211862 | |
| 14 | TRAJ29 | T cell receptor alpha joining 29 | 211860 | 211859 | 211861 | |
| 14 | TRAJ3 | T cell receptor alpha joining 3 | 211886 | 211885 | 211887 | |
| 14 | TRAJ30 | T cell receptor alpha joining 30 | 211859 | 211858 | 211860 | |
| 14 | TRAJ31 | T cell receptor alpha joining 31 | 211858 | 211857 | 211859 | |
| 14 | TRAJ32 | T cell receptor alpha joining 32 | 211857 | 211856 | 211858 | |
| 14 | TRAJ33 | T cell receptor alpha joining 33 | 211856 | 211855 | 211857 | |
| 14 | TRAJ34 | T cell receptor alpha joining 34 | 211855 | 211854 | 211856 | |
| 14 | TRAJ35 | T cell receptor alpha joining 35 (non-functional) | 211854 | 276699 | 211855 | |
| 14 | TRAJ36 | T cell receptor alpha joining 36 | 276699 | 278661 | 211854 | |
| 14 | TRAJ37 | T cell receptor alpha joining 37 | 278661 | 211851 | 276699 | |
| 14 | TRAJ38 | T cell receptor alpha joining 38 | 211851 | 211850 | 278661 | |
| 14 | TRAJ39 | T cell receptor alpha joining 39 | 211850 | 211849 | 211851 | |
| 14 | TRAJ4 | T cell receptor alpha joining 4 | 211885 | 211884 | 211886 | |
| 14 | TRAJ40 | T cell receptor alpha joining 40 | 211849 | 211848 | 211850 | |
| 14 | TRAJ41 | T cell receptor alpha joining 41 | 211848 | 211847 | 211849 | |
| 14 | TRAJ42 | T cell receptor alpha joining 42 | 211847 | 211846 | 211848 | |
| 14 | TRAJ43 | T cell receptor alpha joining 43 | 211846 | 211845 | 211847 | |
| 14 | TRAJ44 | T cell receptor alpha joining 44 | 211845 | 211844 | 211846 | |
| 14 | TRAJ45 | T cell receptor alpha joining 45 | 211844 | 211843 | 211845 | |
| 14 | TRAJ46 | T cell receptor alpha joining 46 | 211843 | 211842 | 211844 | |
| 14 | TRAJ47 | T cell receptor alpha joining 47 | 211842 | 211841 | 211843 | |
| 14 | TRAJ48 | T cell receptor alpha joining 48 | 211841 | 211840 | 211842 | |
| 14 | TRAJ49 | T cell receptor alpha joining 49 | 211840 | 211839 | 211841 | |
| 14 | TRAJ5 | T cell receptor alpha joining 5 | 211884 | 211883 | 211885 | |
| 14 | TRAJ50 | T cell receptor alpha joining 50 | 211839 | 211838 | 211840 | |
| 14 | TRAJ52 | T cell receptor alpha joining 52 | 211838 | 211837 | 211839 | |
| 14 | TRAJ53 | T cell receptor alpha joining 53 | 211837 | 211836 | 211838 | |
| 14 | TRAJ54 | T cell receptor alpha joining 54 | 211836 | 211835 | 211837 | |
| 14 | TRAJ56 | T cell receptor alpha joining 56 | 211835 | 211834 | 211836 | |
| 14 | TRAJ57 | T cell receptor alpha joining 57 | 211834 | 211833 | 211835 | |
| 14 | TRAJ58 | T cell receptor alpha joining 58 (non-functional) | 211833 | 211832 | 211834 | |
| 14 | TRAJ59 | T cell receptor alpha joining 59 (non-functional) | 211832 | 211831 | 211833 | |
| 14 | TRAJ6 | T cell receptor alpha joining 6 | 211883 | 211882 | 211884 | |
| 14 | TRAJ61 | T cell receptor alpha joining 61 (non-functional) | 211831 | 256590 | 211832 | |
| 14 | TRAJ7 | T cell receptor alpha joining 7 | 211882 | 211880 | 211883 | |
| 14 | TRAJ9 | T cell receptor alpha joining 9 | 211880 | 211879 | 211882 | |
| 14 | TRAV10 | T cell receptor alpha variable 10 | 211784 | 211783 | 211785 | |
| 14 | TRAV1-1 | T cell receptor alpha variable 1-1 | 255569 | 169208 | 255582 | |
| 14 | TRAV1-2 | T cell receptor alpha variable 1-2 | 256553 | 255582 | 221977 | |
| 14 | TRAV12-1 | T cell receptor alpha variable 12-1 | 211785 | 211784 | 211786 | |
| 14 | TRAV12-2 | T cell receptor alpha variable 12-2 | 211789 | 211788 | 211790 | 30, 37, |
| 14 | TRAV12-3 | T cell receptor alpha variable 12-3 | 211794 | 211793 | 211795 | |
| 14 | TRAV13-1 | T cell receptor alpha variable 13-1 | 211788 | 211787 | 211789 | |
| 14 | TRAV13-2 | T cell receptor alpha variable 13-2 | 211791 | 211790 | 211792 | |
| 14 | TRAV14DV4 | T cell receptor alpha variable 14/delta variable 4 | 211792 | 211791 | 211793 | |
| 14 | TRAV16 | T cell receptor alpha variable 16 | 211796 | 211795 | 211797 | |
| 14 | TRAV17 | T cell receptor alpha variable 17 | 211797 | 211796 | 211798 | |
| 14 | TRAV18 | T cell receptor alpha variable 18 | 211798 | 211797 | 211799 | |
| 14 | TRAV19 | T cell receptor alpha variable 19 | 211799 | 211798 | 211800 | |
| 14 | TRAV2 | T cell receptor alpha variable 2 | 211776 | 221977 | 211777 | |
| 14 | TRAV20 | T cell receptor alpha variable 20 | 211800 | 211799 | 211801 | |
| 14 | TRAV21 | T cell receptor alpha variable 21 | 211801 | 211800 | 211802 | |
| 14 | TRAV22 | T cell receptor alpha variable 22 | 211802 | 211801 | 211803 | |
| 14 | TRAV23DV6 | T cell receptor alpha variable 23/delta variable 6 | 211803 | 211802 | 211804 | |
| 14 | TRAV24 | T cell receptor alpha variable 24 | 211805 | 211804 | 211806 | |
| 14 | TRAV25 | T cell receptor alpha variable 25 | 211806 | 211805 | 211807 | |
| 14 | TRAV26-1 | T cell receptor alpha variable 26-1 | 211807 | 211806 | 211808 | |
| 14 | TRAV26-2 | T cell receptor alpha variable 26-2 | 211812 | 259092 | 277734 | |
| 14 | TRAV27 | T cell receptor alpha variable 27 | 211809 | 211808 | 211810 | |
| 14 | TRAV29DV5 | T cell receptor alpha variable 29/delta variable 5 (gene/pseudogene) | 211810 | 211809 | 259092 | |
| 14 | TRAV3 | T cell receptor alpha variable 3 (gene/pseudogene) | 211777 | 211776 | 211778 | |
| 14 | TRAV30 | T cell receptor alpha variable 30 | 259092 | 211810 | 211812 | |
| 14 | TRAV34 | T cell receptor alpha variable 34 | 211813 | 277734 | 211815 | |
| 14 | TRAV36DV7 | T cell receptor alpha variable 36/delta variable 7 | 211815 | 211813 | 211816 | |
| 14 | TRAV38-1 | T cell receptor alpha variable 38-1 | 211816 | 211815 | 211829 | |
| 14 | TRAV38-2DV8 | T cell receptor alpha variable 38-2/delta variable 8 | 211817 | 211829 | 211818 | |
| 14 | TRAV39 | T cell receptor alpha variable 39 | 211818 | 211817 | 211819 | |
| 14 | TRAV4 | T cell receptor alpha variable 4 | 211778 | 211777 | 211779 | |
| 14 | TRAV40 | T cell receptor alpha variable 40 | 211819 | 211818 | 211820 | |
| 14 | TRAV41 | T cell receptor alpha variable 41 | 211820 | 211819 | 211821 | |
| 14 | TRAV5 | T cell receptor alpha variable 5 | 211779 | 211778 | 211780 | |
| 14 | TRAV6 | T cell receptor alpha variable 6 | 211780 | 211779 | 211781 | |
| 14 | TRAV7 | T cell receptor alpha variable 7 | 211781 | 211780 | 211782 | |
| 14 | TRAV8-1 | T cell receptor alpha variable 8-1 | 211782 | 211781 | 211783 | |
| 14 | TRAV8-2 | T cell receptor alpha variable 8-2 | 211786 | 211785 | 211787 | |
| 14 | TRAV8-3 | T cell receptor alpha variable 8-3 | 211787 | 211786 | 211788 | |
| 14 | TRAV8-4 | T cell receptor alpha variable 8-4 | 211790 | 211789 | 211791 | |
| 14 | TRAV8-6 | T cell receptor alpha variable 8-6 | 211795 | 211794 | 211796 | |
| 14 | TRAV8-7 | T cell receptor alpha variable 8-7 (non-functional) | 211808 | 211807 | 211809 | |
| 14 | TRAV9-1 | T cell receptor alpha variable 9-1 | 211783 | 211782 | 211784 | |
| 14 | TRAV9-2 | T cell receptor alpha variable 9-2 | 211793 | 211792 | 211794 | |
| 14 | TRDC | T cell receptor delta constant | 211829 | 211816 | 211817 | |
| 14 | TRDD1 | T cell receptor delta diversity 1 | 223997 | 211821 | 237235 | |
| 14 | TRDD2 | T cell receptor delta diversity 2 | 237235 | 223997 | 228985 | |
| 14 | TRDD3 | T cell receptor delta diversity 3 | 228985 | 237235 | 211825 | |
| 14 | TRDJ1 | T cell receptor delta joining 1 | 211825 | 228985 | 211826 | |
| 14 | TRDJ2 | T cell receptor delta joining 2 | 211827 | 211826 | 211828 | |
| 14 | TRDJ3 | T cell receptor delta joining 3 | 211828 | 211827 | 256590 | |
| 14 | TRDJ4 | T cell receptor delta joining 4 | 211826 | 211825 | 211827 | |
| 14 | TRDV1 | T cell receptor delta variable 1 | 211804 | 211803 | 211805 | 39, 36, 22 |
| 14 | TRDV2 | T cell receptor delta variable 2 | 211821 | 211820 | 223997 | |
| 14 | TRDV3 | T cell receptor delta variable 3 | 256590 | 211828 | 211831 | |
| 14 | TC2N | tandem C2 domains, nuclear | 165929 | 133962 | 140092 | 42, 4, 29 |
| 14 | TBPL2 | TATA-box binding protein like 2 | 182521 | 126775 | 126777 | 38, |
| 14 | TCL1A | T-cell leukemia/lymphoma 1A | 100721 | 213231 | 250366 | 21, 41, 22 |
| 14 | TCL1B | T-cell leukemia/lymphoma 1B | 213231 | 182512 | 100721 | 21, 22, 23 |
| 14 | TRAC | T-cell receptor alpha constant | 277734 | 211812 | 211813 | |
| 14 | TUNAR | TCL1 upstream neural differentiation-associated RNA | 250366 | 100721 | 227051 | 14, 8, 38 |
| 14 | TECPR2 | tectonin beta-propeller repeat containing 2 | 196663 | 100865 | 156381 | 34, 29, 18 |
| 14 | TEP1 | telomerase associated protein 1 | 129566 | 129484 | 185271 | 35, 31, 18 |
| 14 | TINF2 | TERF1 interacting nuclear factor 2 | 092330 | 100938 | 092295 | 29, 11, 27 |
| 14 | TEX22 | testis expressed 22 | 226174 | 179364 | 182979 | 21, 2, 38 |
| 14 | TSSK4 | testis specific serine kinase 4 | 139908 | 254692 | 254505 | 38, 4, 39 |
| 14 | TTC5 | tetratricopeptide repeat domain 5 | 136319 | 176198 | 100814 | |
| 14 | TTC6 | tetratricopeptide repeat domain 6 | 139865 | 129514 | 139874 | |
| 14 | TTC7B | tetratricopeptide repeat domain 7B | 165914 | 198668 | 100784 | 26, 11, 32 |
| 14 | TTC8 | tetratricopeptide repeat domain 8 | 165533 | 165521 | 053254 | 33, 2, 29 |
| 14 | TTC9 | tetratricopeptide repeat domain 9 | 133985 | 133997 | 006432 | 31, 32, 30 |
| 14 | THTPA | thiamine triphosphatase | 259431 | 136367 | 213983 | 40, 39, 33 |
| 14 | TXNDC16 | thioredoxin domain containing 16 | 087301 | 125384 | 180998 | 22, 42, 38 |
| 14 | TMX1 | thioredoxin related transmembrane protein 1 | 139921 | 100505 | 139926 | 45, 42, 13 |
| 14 | TRIP11 | thyroid hormone receptor interactor 11 | 100815 | 140092 | 066427 | 1, 30, 29 |
| 14 | TSHR | thyroid stimulating hormone receptor | 165409 | 100629 | 165417 | 40, 39, 27 |
| 14 | TNFAIP2 | TNF alpha induced protein 2 | 185215 | 205436 | 100664 | |
| 14 | TRAF3 | TNF receptor associated factor 3 | 131323 | 089902 | 166126 | 3, 22, 5 |
| 14 | TOX4 | TOX high mobility group box family member 4 | 092203 | 129472 | 165819 | 9, 3, 24 |
| 14 | TRAPPC6B | trafficking protein particle complex 6B | 182400 | 092208 | 100941 | 39, 26, 33 |
| 14 | TGFB3 | transforming growth factor beta 3 | 119699 | 119650 | 089916 | 15, 1, 26 |
| 14 | TGM1 | transglutaminase 1 | 092295 | 092330 | 100949 | 18, 24, 13 |
| 14 | L3HYPDH | trans-L-3-hydroxyproline dehydratase | 126790 | 181619 | 050130 | |
| 14 | TIMM9 | translocase of inner mitochondrial membrane 9 | 100575 | 196860 | 100578 | 1, 29, 4 |
| 14 | TOMM20L | translocase of outer mitochondrial membrane 20 like | 196860 | 032219 | 100575 | 6, 27, 33 |
| 14 | TM9SF1 | transmembrane 9 superfamily member 1 | 100926 | 259522 | 254692 | 32, 9, 4 |
| 14 | TMED10 | transmembrane p24 trafficking protein 10 | 170348 | 119638 | 170345 | 42, 30, 29 |
| 14 | TMED8 | transmembrane p24 trafficking protein family member 8 | 100580 | 100577 | 100583 | |
| 14 | TMEM121 | transmembrane protein 121 | 184986 | 185347 | 211890 | 33, 23, 8 |
| 14 | TMEM179 | transmembrane protein 179 | 258986 | 066735 | 184601 | 20, 8, 14 |
| 14 | TMEM229B | transmembrane protein 229B | 198133 | 100558 | 054690 | 29, 35, 40 |
| 14 | TMEM251 | transmembrane protein 251 | 153485 | 165943 | 259066 | 35, 3, 29 |
| 14 | TMEM253 | transmembrane protein 253 | 232070 | 165804 | 169327 | 27, 3, 19 |
| 14 | TMEM260 | transmembrane protein 260 | 070269 | 139946 | 165588 | 22, 11, 24 |
| 14 | TMEM30B | transmembrane protein 30B | 182107 | 027075 | 258989 | |
| 14 | TMEM55B | transmembrane protein 55B | 165782 | 100823 | 198805 | 3, 26, 31 |
| 14 | TMEM63C | transmembrane protein 63C | 165548 | 259164 | 177108 | |
| 14 | TRIM9 | tripartite motif containing 9 | 100505 | 131969 | 139921 | 8, 31, 26 |
| 14 | TRMT5 | tRNA methyltransferase 5 | 126814 | 020426 | 139974 | 1, 13, 24 |
| 14 | TRMT61A | tRNA methyltransferase 61A | 166166 | 166165 | 166170 | 41, 34, 1 |
| 14 | WARS | tryptophanyl-tRNA synthetase | 140105 | 140107 | 176473 | 36, 9, 45 |
| 14 | TPPP2 | tubulin polymerization promoting protein family member 2 | 179636 | 165795 | 206150 | 38, |
| 14 | TTLL5 | tubulin tyrosine ligase like 5 | 119685 | 119686 | 133935 | 43, 38, 10 |
| 14 | TDRD9 | tudor domain containing 9 | 156414 | 156411 | 227729 | 40, 28, 29 |
| 14 | TDP1 | tyrosyl-DNA phosphodiesterase 1 | 042088 | 140025 | 152315 | 22, 42, 12 |
| 14 | UBR7 | ubiquitin protein ligase E3 component n-recognin 7 (putative) | 012963 | 170270 | 011114 | 26, 39, 5 |
| 14 | UNC79 | unc-79 homolog (C. elegans) | 133958 | 011114 | 187581 | 26, 31, 8 |
| 14 | VRK1 | vaccinia related kinase 1 | 100749 | 090060 | 176605 | 4, 7, 10 |
| 14 | VCPKMT | valosin containing protein lysine methyltransferase | 100483 | 165527 | 100485 | 5, 29, 22 |
| 14 | VASH1 | vasohibin 1 | 071246 | 119715 | 013523 | 18, 45, 20 |
| 14 | VRTN | vertebrae development associated | 133980 | 119688 | 183379 | 12, 27, 38 |
| 14 | VTI1B | vesicle transport through interaction with t-SNAREs 1B | 100568 | 081181 | 258466 | 21, 9, 40 |
| 14 | VSX2 | visual system homeobox 2 | 119614 | 205659 | 119688 | 14, 25, 8 |
| 14 | VIPAS39 | VPS33B interacting protein, apical-basolateral polarity | 151445 | 280308 | 100591 | 29, 40, 18 |
| | | regulator, spe-39 homolog | | | | |
| 14 | WDHD1 | WD repeat and HMG-box DNA binding protein 1 | 198554 | 131979 | 180008 | 43, 12, 22 |
| 14 | WDR20 | WD repeat domain 20 | 140153 | 080824 | 080823 | 22, 38, 23 |
| 14 | WDR25 | WD repeat domain 25 | 176473 | 140105 | 183092 | 21, 26, 32 |
| 14 | WDR89 | WD repeat domain 89 | 140006 | 154001 | 126821 | 2, 45, 42 |
| 14 | XRCC3 | X-ray repair cross complementing 3 | 126215 | 256500 | 100711 | 21, 3, 41 |
| 14 | YLPM1 | YLP motif containing 1 | 119596 | 119616 | 119608 | 18, 16, 40 |
| 14 | YY1 | YY1 transcription factor | 100811 | 168350 | 197119 | |
| 14 | ZFP36L1 | ZFP36 ring finger protein like 1 | 185650 | 182185 | 072110 | 44, 43, 29 |
| 14 | ZBTB1 | zinc finger and BTB domain containing 1 | 126804 | 179841 | 126803 | 22, 6, 9 |
| 14 | ZBTB25 | zinc finger and BTB domain containing 25 | 089775 | 100714 | 179841 | |
| 14 | ZBTB42 | zinc finger and BTB domain containing 42 | 179627 | 142208 | 099814 | 3, 34, 23 |
| 14 | ZC2HC1C | zinc finger C2HC-type containing 1C | 119703 | 119640 | 119638 | 38, 21, 44 |
| 14 | ZC3H14 | zinc finger CCCH-type containing 14 | 100722 | 070778 | 165521 | 38, 4, 37 |
| 14 | ZDHHC22 | zinc finger DHHC-type containing 22 | 177108 | 165548 | 165553 | 14, 8, 15 |
| 14 | ZFYVE1 | zinc finger FYVE-type containing 1 | 165861 | 119599 | 119707 | 24, 43, 26 |
| 14 | ZFYVE21 | zinc finger FYVE-type containing 21 | 100711 | 126215 | 088808 | 34, 26, 1 |
| 14 | ZFYVE26 | zinc finger FYVE-type containing 26 | 072121 | 139988 | 182185 | 43, 21, 18 |
| 14 | ZFHX2 | zinc finger homeobox 2 | 136367 | 129460 | 259431 | 2, 28, 20 |
| 14 | ZNF219 | zinc finger protein 219 | 165804 | 165801 | 232070 | 26, 39, 37 |
| 14 | ZNF410 | zinc finger protein 410 | 119725 | 258653 | 156050 | 36, 30, 45 |
| 14 | ZNF839 | zinc finger protein 839 | 022976 | 080823 | 100865 | 34, 26, 41 |

Table 7 provides the gene information for the genes found on Chromosomes 15-17.

TABLE 7
Genes on Chromosomes 15-17

| | | | | Up | Down | Tissue |
|---|---|---|---|---|---|---|
| Chr. | Gene Symbol | Gene Name | ENSG | ENSG | ENSG | Code |
| 15 | HACD3 | 3-hydroxyacyl-CoA dehydratase 3 | 074696 | 074603 | 138614 | |
| 15 | ABHD17C | abhydrolase domain containing 17C | 136379 | 172379 | 103888 | 11, 4, 16 |
| 15 | ABHD2 | abhydrolase domain containing 2 | 140526 | 140545 | 140522 | 24, 38, 33 |
| 15 | ANP32A | acidic nuclear phosphoprotein 32 family member A | 140350 | 103647 | 258484 | 21, 4, 18 |
| 15 | ACTC1 | actin, alpha, cardiac muscle 1 | 159251 | 159248 | 021776 | 15, 43, 25 |
| 15 | ARPIN | actin-related protein 2/3 complex inhibitor | 242498 | 250021 | 140548 | |
| 15 | ACSBG1 | acyl-CoA synthetase bubblegum family member 1 | 103740 | 166411 | 140403 | 26, 8, 35 |
| 15 | ADAM10 | ADAM metallopeptidase domain 10 | 137845 | 166035 | 128923 | 42, 39, 45 |
| 15 | ADAMTS17 | ADAM metallopeptidase with thrombospondin type 1 motif 17 | 140470 | 183060 | 154227 | 39, 11, 15 |
| 15 | ADAMTS7 | ADAM metallopeptidase with thrombospondin type 1 motif 7 | 136378 | 117971 | 185787 | 15, 12, 37 |
| 15 | ADAMTSL3 | ADAMTS like 3 | 156218 | 140600 | 259511 | 1, 42, 40 |
| 15 | AP3B2 | adapter related protein complex 3 beta 2 subunit | 103723 | 214575 | 186628 | 29, 31, 14 |
| 15 | AP3S2 | adapter related protein complex 3 sigma 2 subunit | 157823 | 166825 | 250021 | 13, 21, 34 |
| 15 | AP4E1 | adapter related protein complex 4 epsilon 1 subunit | 081014 | 138600 | 183578 | 18, 39, 4 |
| 15 | ADAL | adenosine deaminase like | 168803 | 168806 | 140265 | 24, 9, 2 |
| 15 | ADPGK | ADP dependent glucokinase | 159322 | 140463 | 067141 | 29, 34, 30 |
| 15 | ACAN | aggrecan | 157766 | 172183 | 140511 | 10, 6, 42 |
| 15 | AKAP13 | A-kinase anchoring protein 13 | 170776 | 073417 | 183655 | 5, 32, |
| 15 | ANPEP | alanyl aminepeptidase, membrane | 166825 | 188095 | 157823 | 16, 19, 37 |
| 15 | ALDH1A2 | aldehyde dehydrogenase 1 family member A2 | 128918 | 255529 | 103569 | |
| 15 | ALDH1A3 | aldehyde dehydrogenase 1 family member A3 | 184254 | 279588 | 154237 | 42, 33, 23 |
| 15 | AAGAB | alpha- and gamma-adaptin binding protein | 103591 | 166949 | 103599 | 13, 29, 43 |
| 15 | ALPK3 | alpha kinase 3 | 136383 | 166716 | 156222 | 43, 25, 26 |
| 15 | APBA2 | amyloid beta precursor protein binding family A member 2 | 034053 | 261649 | 104059 | |
| 15 | ANKDD1A | ankyrin repeat and death domain containing 1A | 166839 | 241839 | 090487 | 2, 17, 45 |
| 15 | ASB7 | ankyrin repeat and SOCS box containing 7 | 183475 | 140471 | 279588 | 29, 13, 22 |
| 15 | ANKRD34C | ankyrin repeat domain 34C | 235711 | 058335 | 166557 | 25, 8, |
| 15 | ANKRD63 | ankyrin repeat domain 63 | 230778 | 137841 | 188549 | 27, |
| 15 | ANXA2 | annexin A2 | 182718 | 171956 | 128915 | 43, 4, 6 |
| 15 | APH1B | aph-1 homolog B, gamma-secretase subunit | 138613 | 166128 | 074410 | 38, 42, 14 |
| 15 | AVEN | apoptosis and caspase activation inhibitor | 169857 | 198838 | 184984 | 3, 31, 22 |
| 15 | AEN | apoptosis enhancing nuclease | 181026 | 140543 | 172183 | 9, 35, 12 |
| 15 | AQP9 | aquaporin 9 | 103569 | 128918 | 166035 | 5, 19, 36 |
| 15 | AQR | aquarius intron-binding spliceosomal factor | 021776 | 159251 | 198146 | 31, 1, 42 |
| 15 | ARIH1 | ariadne RBR E3 ubiquitin protein ligase 1 | 166233 | 187806 | 215186 | 9, 6, 4 |
| 15 | ARRDC4 | arrestin domain containing 4 | 140450 | 185594 | 185087 | 43, 11, 19 |
| 15 | ARNT2 | aryl hydrocarbon receptor nuclear translocator 2 | 172379 | 259417 | 136379 | 29, 26, 8 |
| 15 | AGBL1 | ATP/GTP binding protein like 1 | 166748 | 273540 | 140538 | 33, 10, 28 |
| 15 | AGBL1 | ATP/GTP binding protein like 1 | 273540 | 183655 | 166748 | 33, 10, 28 |
| 15 | ATP10A | ATPase phospholipid transporting 10A (putative) | 206190 | 114062 | 166206 | 6, 35, 40 |
| 15 | ATP8B4 | ATPase phospholipid transporting 8B4 (putative) | 104043 | 104047 | 140284 | 5, 42, 7 |
| 15 | ARID3B | AT-rich interaction domain 3B | 179361 | 138629 | 179335 | 12, |
| 15 | BBS4 | Bardet-Biedl syndrome 4 | 140463 | 175202 | 159322 | 4, 30, 45 |
| 15 | BNC1 | basonudin 1 | 169594 | 166503 | 140600 | 38, 9, 16 |
| 15 | BNIP2 | BCL2 interacting protein 2 | 140299 | 140307 | 171956 | 42, 4, 24 |
| 15 | BCL2L10 | BCL2 like 10 | 137875 | 069956 | 069966 | 44, 28, 17 |
| 15 | BMF | Bcl2 modifying factor | 104081 | 140319 | 156970 | 21, 18, 5 |
| 15 | BCL2A1 | BCL2 related protein A1 | 140379 | 180953 | 086666 | 5, 22, 10 |
| 15 | B2M | beta-2-microglobulin | 166710 | 229474 | 185880 | 30, 5, 7 |
| 15 | BLOC1S6 | biogenesis of lysosomal organelles complex 1 subunit 6 | 104164 | 104154 | 260170 | 22, 42, 13 |
| 15 | BLM | Bloom syndrome RecQ like helicase | 197299 | 140577 | 140564 | 22, 41, 34 |
| 15 | BAHD1 | bromo adjacent homology domain containing 1 | 140320 | 128928 | 169105 | 41, 1, 35 |
| 15 | BTBD1 | BTB domain containing 1 | 064726 | 169609 | 136404 | 18, 37, 6 |
| 15 | BUB1B | BUB1 mitotic checkpoint serine/threonine kinase B | 156970 | 104081 | 259288 | 12, 24, 39 |
| 15 | BUB1B-PAK6 | BUB1B-PAK6 readthrough | 259288 | 156970 | 137843 | |
| 15 | C15orf38-AP3S2 | C15orf38-AP3S2 readthrough | 250021 | 157823 | 242498 | |
| 15 | C2CD4A | C2 calcium dependent domain containing 4A | 198535 | 129003 | 205502 | 37, 28, 20 |
| 15 | C2CD4B | C2 calcium dependent domain containing 4B | 205502 | 198535 | 171914 | 28, 26, 20 |
| 15 | CHP1 | calcineurin like EF-hand protein 1 | 187446 | 178997 | 104147 | 11, 42, 24 |
| 15 | CIB1 | calcium and integrin binding 1 | 185043 | 185033 | 183208 | 3, 34, 41 |
| 15 | CIB2 | calcium and integrin binding family member 2 | 136425 | 183476 | 166411 | 34, 41, 20 |
| 15 | CALML4 | calmodulin like 4 | 129007 | 260007 | 128973 | 3, 42, 16 |
| 15 | CAPN3 | calpain 3 | 092529 | 214013 | 103994 | 35, 25, 24 |
| 15 | ARPP19 | cAMP regulated phosphoprotein 19 | 128989 | 280817 | 047346 | 8, 11, 2 |
| 15 | CASC4 | cancer susceptibility candidate 4 | 166734 | 171877 | 137770 | |
| 15 | CT62 | cancer/testis antigen 62 | 225362 | 187720 | 278570 | 38, 10, 8 |
| 15 | CHST14 | carbohydrate sulfotransferase 14 | 169105 | 140320 | 128891 | 11, 12, 10 |
| 15 | CA12 | carbonic anhydrase 12 | 074410 | 138613 | 140455 | 17, 14, 6 |
| 15 | CILP | cartilage intermediate layer protein | 138615 | 166855 | 138617 | 10, 42, 43 |
| 15 | CSNK1G1 | casein kinase 1 gamma 1 | 169118 | 166794 | 259316 | 23, 22, 38 |
| 15 | CLPX | caseinolytic mitochondrial matrix peptidase chaperone subunit | 166855 | 090470 | 138615 | 26, 7, 29 |
| 15 | CTSH | cathepsin H | 103811 | 185787 | 058335 | 22, 37, 41 |
| 15 | CATSPER2 | cation channel sperm associated 2 | 166762 | 242866 | 223572 | 26, 38, 43 |
| 15 | CD276 | CD276 molecule | 103855 | 156642 | 205363 | |
| 15 | CLK3 | CDC like kinase 3 | 179335 | 179361 | 179151 | 1, 29, 24 |
| 15 | CCPG1 | cell cycle progression 1 | 260916 | 069943 | 261652 | 42, 1, 10 |
| 15 | CEMIP | cell migration inducing hyaluronan binding protein | 103888 | 136379 | 117899 | 6, 11, 10 |
| 15 | CRABP1 | cellular retinoic acid binding protein 1 | 166426 | 140395 | 136381 | 14, 27, 31 |
| 15 | CEP152 | centrosomal protein 152 | 103995 | 166147 | 185634 | 19, 6, 12 |
| 15 | CERS3 | ceramide synthase 3 | 154227 | 140470 | 140471 | 38, 24, 35 |
| 15 | CLN6 | ceroid-lipofuscinosis, neuronal 6, late infantile, variant | 128973 | 129007 | 169018 | 18, 9, 41 |
| 15 | CHAC1 | ChaC glutathione specific gamma-glutamylcyclotransferase 1 | 128965 | 128917 | 128908 | 9, 21, 3 |
| 15 | CHRM5 | cholinergic receptor muscarinic 5 | 184984 | 169857 | 134153 | 38, 44, 8 |
| 15 | CHRNA3 | cholinergic receptor nicotinic alpha 3 subunit | 080644 | 169684 | 117971 | 26, 39, 14 |
| 15 | CHRNA5 | cholinergic receptor nicotinic alpha 5 subunit | 169684 | 041357 | 080644 | 4, 26, 31 |
| 15 | CHRNA7 | cholinergic receptor nicotinic alpha 7 subunit | 175344 | 169918 | 249931 | |
| 15 | CHRNB4 | cholinergic receptor nicotinic beta 4 subunit | 117971 | 080644 | 136378 | 26, 38, 44 |
| 15 | CSPG4 | chondroitin sulfate proteoglycan 4 | 173546 | 173548 | 182950 | 35, 26, 6 |
| 15 | CHSY1 | chondroitin sulfate synthase 1 | 131873 | 154237 | 131871 | |
| 15 | CHRFAM7A | CHRNA7 (exons 5-10) and FAM7A (exons A-E) fusion | 166664 | 261247 | 186399 | 29, 40, 26 |
| 15 | CHD2 | chromodomain helicase DNA binding protein 2 | 173575 | 279765 | 182175 | 30, 18, 19 |
| 15 | C15orf39 | chromosome 15 open reading frame 39 | 167173 | 138621 | 140478 | 38, 7, 22 |
| 15 | C15orf40 | chromosome 15 open reading frame 40 | 169609 | 169612 | 064726 | 7, 28, 18 |
| 15 | C15orf41 | chromosome 15 open reading frame 41 | 186073 | 134146 | 134138 | 4, 34, 10 |
| 15 | C15orf48 | chromosome 15 open reading frame 48 | 166920 | 171763 | 104154 | 38, 28, 16 |
| 15 | C15orf52 | chromosome 15 open reading frame 52 | 188549 | 230778 | 233041 | 4, 25, 26 |
| 15 | C15orf53 | chromosome 15 open reading frame 53 | 175779 | 172575 | 137801 | 36, |
| 15 | C15orf57 | chromosome 15 open reading frame 57 | 128891 | 169105 | 166133 | 30, 17, 37 |
| 15 | C15orf59 | chromosome 15 open reading frame 59 | 205363 | 103855 | 167139 | 16, 14, |
| 15 | C15orf61 | chromosome 15 open reading frame 61 | 189227 | 103599 | 137764 | 38, 33, |
| 15 | C15orf62 | chromosome 15 open reading frame 62 | 188277 | 104129 | 166140 | 13, 24, 22 |
| 15 | C15orf65 | chromosome 15 open reading frame 65 | 261652 | 260916 | 256061 | |
| 15 | CFAP161 | cilia and flagella associated protein 161 | 156206 | 140406 | 172349 | |
| 15 | CGNL1 | cingulin like 1 | 128849 | 140262 | 137878 | 40, 1, 32 |
| 15 | CDAN1 | codanin 1 | 140326 | 159433 | 128881 | 27, 40, 44 |
| 15 | CCDC33 | coiled-coil domain containing 33 | 140481 | 137868 | 140459 | |
| 15 | COMMD4 | COMM domain containing 4 | 140365 | 167195 | 140398 | 34, 41, 38 |
| 15 | CPLX3 | complexin 3 | 213578 | 140506 | 140474 | 14, 26, 36 |
| 15 | COPS2 | COP9 signalosome subunit 2 | 166200 | 138593 | 156958 | 1, 13, 45 |
| 15 | CORO2B | coronin 2B | 103647 | 137809 | 140350 | 26, 8, 18 |
| 15 | CTXN2 | cortexin 2 | 233932 | 104177 | 074803 | 26, 14, |
| 15 | CKMT1A | creatine kinase, mitochondrial 1A | 223572 | 166762 | 167004 | 26, 31, 30 |
| 15 | CKMT1B | creatine kinase, mitochondrial 1B | 237289 | 168781 | 242866 | 8, 37, 13 |
| 15 | CRTC3 | CREB regulated transcription coactivator 3 | 140577 | 140575 | 197299 | |
| 15 | CSK | c-src tyrosine kinase | 103653 | 140505 | 140506 | 41, 36, 5 |
| 15 | CTDSPL2 | CTD small phosphatase like 2 | 137770 | 166734 | 104131 | 42, 22, 7 |
| 15 | CELF6 | CUGBP, Elav-like family member 6 | 140488 | 273025 | 213614 | 26, 31, 15 |
| 15 | CCNB2 | cyclin B2 | 157456 | 137776 | 157483 | 13, 3, 4 |
| 15 | CCNDBP1 | cyclin D1 binding protein 1 | 166946 | 137842 | 104055 | 3, 11, 1 |
| 15 | COX5A | cytochrome c oxidase subunit 5A | 178741 | 178761 | 178718 | 25, 22, 16 |
| 15 | CYP1A1 | cytochrome P450 family 1 subfamily A member 1 | 140465 | 179151 | 140505 | 1, 42, 13 |
| 15 | CYP1A2 | cytochrome P450 family 1 subfamily A member 2 | 140505 | 140465 | 103653 | 19, 23, |
| 15 | CYP11A1 | cytochrome P450 family 11 subfamily A member 1 | 140459 | 140481 | 138623 | 2, 32, 9 |
| 15 | CYP19A1 | cytochrome P450 family 19 subfamily A member 1 | 137869 | 183578 | 186417 | 32, 27, 45 |
| 15 | CYFIP1 | cytoplasmic FMR1 interacting protein 1 | 273749 | 140157 | 275835 | 24, 6, 29 |
| 15 | CPEB1 | cytoplasmic polyadenylation element binding protein 1 | 214575 | 260836 | 103723 | |
| 15 | DAPK2 | death associated protein kinase 2 | 035664 | 103657 | 166797 | 38, |
| 15 | DET1 | de-etiolated homolog 1 (Arabidopsis) | 140543 | 181991 | 181026 | 18, 36, 33 |
| 15 | DLL4 | delta like canonical Notch ligand 4 | 128917 | 104142 | 128965 | 32, 16, 2 |
| 15 | DENND4A | DENN domain containing 4A | 174485 | 074621 | 103769 | 22, 26, 9 |
| 15 | DUT | deoxyuridine triphosphatase | 128951 | 074803 | 166147 | 22, 4, 11 |
| 15 | DPP8 | dipeptidyl peptidase 8 | 074603 | 103742 | 074696 | 34, 24, 38 |
| 15 | PPIP5K1 | diphosphoinositol pentakisphosphate kinase 1 | 168781 | 166963 | 237289 | 40, 18, 22 |
| 15 | DPH6 | diphthamine biosynthesis 6 | 134146 | 255192 | 186073 | 4, 36, 3 |
| 15 | DIS3L | DIS3 like exosome 3′-5′ exoribonuclease | 166938 | 157890 | 075131 | 9, 2, 25 |
| 15 | DISP2 | dispatched RND transporter family member 2 | 140323 | 233041 | 128944 | 20, |
| 15 | DMXL2 | Dmx like 2 | 104093 | 186417 | 104112 | 45, 31, 5 |
| 15 | POLG | DNA polymerase gamma, catalytic subunit | 140521 | 140525 | 140519 | 41, 1, 35 |
| 15 | DNAJA4 | DnaJ heat shock protein family (Hsp40) member A4 | 140403 | 103740 | 140395 | 42, 26, 31 |
| 15 | DNAJC17 | DnaJ heat shock protein family (Hsp40) member C17 | 104129 | 137880 | 188277 | 40, 37, 34 |
| 15 | DTWD1 | DTW domain containing 1 | 104047 | 140285 | 104043 | 29, 7, 34 |
| 15 | DUOX1 | dual oxidase 1 | 137857 | 140254 | 138606 | 40, 42, 4 |
| 15 | DUOX2 | dual oxidase 2 | 140279 | 140263 | 140274 | 35, 40, 24 |
| 15 | DUOXA1 | dual oxidase maturation factor 1 | 140254 | 140274 | 137857 | 40, 24, 20 |
| 15 | DUOXA2 | dual oxidase maturation factor 2 | 140274 | 140279 | 140254 | 16, 42, 22 |
| 15 | DYX1C1 | dyslexia susceptibility 1 candidate 1 | 256061 | 261652 | 171016 | 4, 31, 40 |
| 15 | EHD4 | EH domain containing 4 | 103966 | 137877 | 188089 | 1, 21, 22 |
| 15 | ETFA | electron transfer flavoprotein alpha subunit | 140374 | 169758 | 159556 | 29, 1, 31 |
| 15 | ELL3 | elongation factor for RNA polymerase II 3 | 128886 | 167004 | 262560 | |
| 15 | EFL1 | elongation factor like GTPase 1 | 140598 | 183496 | 188659 | 10, 43, 18 |
| 15 | EDC3 | enhancer of mRNA decapping 3 | 179151 | 179335 | 140465 | 33, 1, 13 |
| 15 | EID1 | EP300 interacting inhibitor of differentiation 1 | 255302 | 185634 | 138593 | 43, 13, 15 |
| 15 | EMC4 | ER membrane protein complex subunit 4 | 128463 | 134152 | 140199 | 29, 6, 28 |
| 15 | EMC7 | ER membrane protein complex subunit 7 | 134153 | 184984 | 182405 | 18, 31, 29 |
| 15 | EPB42 | erythrocyte membrane protein band 4.2 | 166947 | 159459 | 137842 | 19, 7, 15 |
| 15 | EIF2AK4 | eukaryotic translation initiation factor 2 alpha kinase 4 | 128829 | 283382 | 140319 | 3, 31, 34 |
| 15 | EIF3J | eukaryotic translation initiation factor 3 subunit J | 104131 | 137770 | 104133 | 9, 40, 34 |
| 15 | EXD1 | exonuclease 3′-5′ domain containing 1 | 178997 | 128908 | 187446 | 38, 25, 8 |
| 15 | FAM103A1 | family with sequence similarity 103 member A1 | 169612 | 103942 | 169609 | 29, 4, 1 |
| 15 | FAM169B | family with sequence similarity 169 member B | 185087 | 140450 | 140443 | 16, |
| 15 | FAM174B | family with sequence similarity 174 member B | 185442 | 140557 | 279765 | 4, 28, 31 |
| 15 | FAM189A1 | family with sequence similarity 189 member A1 | 104059 | 034053 | 185115 | 8, 17, 14 |
| 15 | FAM214A | family with sequence similarity 214 member A | 047346 | 128989 | 169856 | 22, 29, 42 |
| 15 | FAM219B | family with sequence similarity 219 member B | 178761 | 178802 | 178741 | 6, 2, 5 |
| 15 | FAM227B | family with sequence similarity 227 member B | 166262 | 156958 | 140285 | 31, 38, 28 |
| 15 | FAM63B | family with sequence similarity 63 member B | 128923 | 137845 | 157450 | 39, 25, 4 |
| 15 | FAM81A | family with sequence similarity 81 member A | 157470 | 171989 | 140297 | |
| 15 | FAM96A | family with sequence similarity 96 member A | 166797 | 035664 | 028528 | 34, 29, 6 |
| 15 | FAM98B | family with sequence similarity 98 member B | 171262 | 166068 | 172575 | 43, 4, 6 |
| 15 | FAN1 | FANCD2 and FANCI associated nuclease 1 | 198690 | 187951 | 166912 | 1, 26, 17 |
| 15 | FANCI | Fanconi anemia complementation group I | 140525 | 140522 | 140521 | 18, 22, 34 |
| 15 | FBXL22 | F-box and leucine rich repeat protein 22 | 197361 | 140455 | 103657 | |
| 15 | FBXO22 | F-box protein 22 | 167196 | 140367 | 169752 | 7, 4, 29 |
| 15 | FEM1B | fem-1 homolog B | 169018 | 128973 | 137809 | 9, 42, 38 |
| 15 | FRMD5 | FERM domain containing 5 | 171877 | 092470 | 166734 | 40, 8, 35 |
| 15 | FES | FES proto-oncogene, tyrosine kinase | 182511 | 140564 | 196547 | 36, 42, 1 |
| 15 | FBN1 | fibrillin 1 | 166147 | 128951 | 103995 | 3, 6, 37 |
| 15 | FGF7 | fibroblast growth factor 7 | 140285 | 166262 | 104047 | 45, 26, 11 |
| 15 | FSD2 | fibronectin type III and SPRY domain containing 2 | 186628 | 103723 | 156232 | 15, 25, 22 |
| 15 | FSIP1 | fibrous sheath interacting protein 1 | 150667 | 137801 | 166073 | 23, 39, 38 |
| 15 | FOXB1 | forkhead box B1 | 171956 | 140299 | 182718 | 38, 17, 12 |
| 15 | FMN1 | formin 1 | 248905 | 166923 | 198838 | 35, 1, 6 |
| 15 | FAH | fumarylacetoacetate hydrolase | 103876 | 086666 | 259417 | 5, 21, 1 |
| 15 | FURIN | furin, paired basic amino acid cleaving enzyme | 140564 | 197299 | 182511 | 1, 9, 34 |
| 15 | GNB5 | G protein subunit beta 5 | 069966 | 137875 | 128833 | 31, 34, 29 |
| 15 | GPR176 | G protein-coupled receptor 176 | 166073 | 150667 | 283382 | |
| 15 | GABPB1 | GA binding protein transcription factor beta subunit 1 | 104064 | 140287 | 138592 | 22, 14, 20 |
| 15 | GALK2 | galactokinase 2 | 156958 | 166200 | 166262 | 36, 2, 13 |
| 15 | GABRA5 | gamma-aminobutyric acid type A receptor alpha5 subunit | 186297 | 166206 | 182256 | 8, 39, 17 |
| 15 | GABRB3 | gamma-aminobutyric acid type A receptor beta3 subunit | 166206 | 206190 | 186297 | |
| 15 | GABRG3 | gamma-aminobutyric acid type A receptor gamma3 subunit | 182256 | 186297 | 104044 | 38, |
| 15 | GJD2 | gap junction protein delta 2 | 159248 | 215252 | 159251 | 14, |
| 15 | GDPGP1 | GDP-D-glucose phosphorylase 1 | 183208 | 185043 | 261147 | 15, 24, 22 |
| 15 | GTF2A2 | general transcription factor IIA subunit 2 | 140307 | 140297 | 140299 | 29, 31, 38 |
| 15 | GLDN | gliomedin | 186417 | 137869 | 104093 | 18, 32, 8 |
| 15 | GCNT3 | glucosaminyl (N-acetyl) transferase 3, mucin type | 140297 | 157470 | 140307 | |
| 15 | GANC | glucosidase alpha, neutral C | 214013 | 103978 | 092529 | 11, 29, 42 |
| 15 | GLCE | glucuronic acid epimerase | 138604 | 255346 | 137819 | 29, 18, 42 |
| 15 | GATM | glycine amidinotransferase | 171766 | 137860 | 171763 | 17, 19, 4 |
| 15 | GOLGA6A | golgin A6 family member A | 159289 | 140464 | 167178 | |
| 15 | GOLGA6B | golgin A6 family member B | 215186 | 166233 | 175202 | |
| 15 | GOLGA6C | golgin A6 family member C | 167195 | 140478 | 140365 | 38, 19, |
| 15 | GOLGA6D | golgin A6 family member D | 140478 | 167173 | 167195 | 38, 35, |
| 15 | GOLGA6L1 | golgin A6 family-like 1 | 273976 | 275835 | 261739 | 38, |
| 15 | GOLGA6L10 | golgin A6 family-like 10 | 278662 | 188659 | 197978 | 18, 19, 23 |
| 15 | GOLGA6L2 | golgin A6 family-like 2 | 174450 | 261739 | 179455 | |
| 15 | GOLGA6L22 | golgin A6 family-like 22 | 277865 | 259261 | 170113 | |
| 15 | GOLGA6L4 | golgin A6 family-like 4 | 184206 | 259511 | 176371 | 25, 12, 15 |
| 15 | GOLGA6L6 | golgin A6 family-like 6 | 277322 | 271336 | 230031 | 38, |
| 15 | GOLGA6L7P | Golgin A6 family-like 7, pseudogene | 261649 | 188626 | 034053 | 38, |
| 15 | GOLGA6L9 | golgin A6 family-like 9 | 197978 | 278662 | 182774 | |
| 15 | GOLGA8A | golgin A8 family member A | 175265 | 176454 | 215252 | 40, 18, 12 |
| 15 | GOLGA8B | golgin A8 family member B | 215252 | 175265 | 159248 | |
| 15 | GOLGA8F | Golgin A8 family member F | 153684 | 128731 | 183629 | 38, 2, 14 |
| 15 | GOLGA8G | Golgin A8 family member G | 183629 | 153684 | 188626 | 38, 2, 14 |
| 15 | GOLGA8H | golgin A8 family member H | 261794 | 178115 | 187951 | |
| 15 | GOLGA8J | golgin A8 family member J | 179938 | 104067 | 261247 | 38, 12, |
| 15 | GOLGA8K | golgin A8 family member K | 249931 | 175344 | 206127 | 30, 38, |
| 15 | GOLGA8M | golgin A8 family member M | 188626 | 183629 | 261649 | |
| 15 | GOLGA8N | golgin A8 family member N | 232653 | 206127 | 198826 | 24, 38, 5 |
| 15 | GOLGA80 | golgin A8 family member O | 206127 | 249931 | 232653 | |
| 15 | GOLGA8Q | Golgin A8 family member Q | 178115 | 186399 | 261794 | |
| 15 | GOLGA8R | golgin A8 family member R | 186399 | 166664 | 178115 | 24, 38, 5 |
| 15 | GOLGA8S | Golgin A8 family members | 261739 | 273976 | 174450 | 38, 40, |
| 15 | GOLGA8T | Golgin A8 family member T | 261247 | 179938 | 166664 | 40, 7, 33 |
| 15 | GRAMD2 | GRAM domain containing 2 | 175318 | 166192 | 067225 | 18, 32, 42 |
| 15 | GREM1 | gremlin 1, DAN family BMP antagonist | 166923 | 166922 | 248905 | 7, 24, 35 |
| 15 | GC0M1 | GRINL1A complex locus 1 | 137878 | 128849 | 263155 | |
| 15 | GCHFR | GTP cyclohydrolase I feedback regulator | 137880 | 137824 | 104129 | 41, 21, 33 |
| 15 | HAUS2 | HAUS augmin like complex subunit 2 | 137814 | 180979 | 159433 | 9, 22, 3 |
| 15 | HDDC3 | HD domain containing 3 | 184508 | 196547 | 140553 | 3, 9, 29 |
| 15 | HERC2 | HECT and RLD domain containing E3 ubiquitin protein ligase 2 | 128731 | 104044 | 153684 | |
| 15 | HERC1 | HECT and RLD domain containing E3 ubiquitin protein ligase family member 1 | 103657 | 197361 | 035664 | 26, 40, 22 |
| 15 | HEXA | hexosaminidase subunit alpha | 213614 | 140488 | 187806 | |
| 15 | HIGD2B | HIG1 hypoxia inducible domain family member 2B | 175202 | 215186 | 140463 | |
| 15 | HMG20A | high mobility group 20A | 140382 | 173517 | 169783 | 42, 39, 38 |
| 15 | HDC | histidine decarboxylase | 140287 | 140284 | 104064 | 1, 39, 30 |
| 15 | HOMER2 | homer scaffolding protein 2 | 103942 | 156232 | 169612 | 2, 40, 33 |
| 15 | HYPK | huntingtin interacting protein K | 242028 | 184716 | 140259 | 2, 11, 25 |
| 15 | HAPLN3 | hyaluronan and proteoglycan link protein 3 | 140511 | 157766 | 140545 | 39, 43, 27 |
| 15 | HYKK | hydroxylysine kinase | 188266 | 136381 | 041357 | 33, 28, 3 |
| 15 | HCN4 | hyperpolarization activated cyclic nucleotide gated potassium channel 4 | 138622 | 067141 | 183324 | 20, 15, 38 |
| 15 | IGHD1OR15-1A | Immunoglobulin heavy diversity 1/OR15-1A (non-functional) | 271336 | 282599 | 277322 | |
| 15 | IGHD1OR15-1B | Immunoglobulin heavy diversity 1/OR15-1B (non-functional) | 270185 | 282268 | 278522 | |
| 15 | IGHD2OR15-2A | Immunoglobulin heavy diversity 2/OR15-2A (non-functional) | 282599 | 282520 | 271336 | |
| 15 | IGHD2OR15-2B | Immunoglobulin heavy diversity 2/OR15-2B (non-functional) | 282268 | 282089 | 270185 | |
| 15 | IGHD3OR15-3A | Immunoglobulin heavy diversity 3/OR15-3A (non-functional) | 282520 | 271317 | 282599 | |
| 15 | IGHD3OR15-3B | Immunoglobulin heavy diversity 3/OR15-3B (non-functional) | 282089 | 270451 | 282268 | |
| 15 | IGHD4OR15-4A | Immunoglobulin heavy diversity 4/OR15-4A (non-functional) | 271317 | 270961 | 282520 | |
| 15 | IGHD4OR15-4B | Immunoglobulin heavy diversity 4/OR15-4B (non-functional) | 270451 | 270824 | 282089 | |
| 15 | IGHD5OR15-5A | Immunoglobulin heavy diversity 5/OR15-5A (non-functional) | 270961 | 259490 | 271317 | |
| 15 | IGHD5OR15-5B | Immunoglobulin heavy diversity 5/OR15-5B (non-functional) | 270824 | 230031 | 270451 | |
| 15 | IGHV1OR15-1 | Immunoglobulin heavy variable 1/OR15-1 (non-functional) | 270505 | 183706 | 259261 | |
| 15 | IGHV1OR15-9 | Immunoglobulin heavy variable 1/OR15-9 (non-functional) | 188403 | 279084 | 259490 | |
| 15 | IGHV3OR15-7 | Immunoglobulin heavy variable 3/OR15-7 (pseudogene) | 259490 | 188403 | 270961 | |
| 15 | IGHV4OR15-8 | Immunoglobulin heavy variable 4/OR15-8 (non-functional) | 259261 | 270505 | 277865 | |
| 15 | ISLR | immunoglobulin superfamily containing leucine rich repeat | 129009 | 167178 | 137868 | 45, 44, 10 |
| 15 | ISLR2 | immunoglobulin superfamily containing leucine rich repeat 2 | 167178 | 159289 | 129009 | 26, 8, 38 |
| 15 | IGDCC3 | immunoglobulin superfamily DCC subclass member 3 | 174498 | 138617 | 103742 | 12, 29, 14 |
| 15 | IGDCC4 | immunoglobulin superfamily DCC subclass member 4 | 103742 | 174498 | 074603 | 40, 32, 36 |
| 15 | IMP3 | IMP3, U3 small nucleolar ribonucleoprotein | 177971 | 169371 | 173548 | 33, 19, 41 |
| 15 | INO80 | INO80 complex subunit | 128908 | 128965 | 178997 | 22, 23, 18 |
| 15 | ITPKA | inosi tol-trisphosphate 3-kinase A | 137825 | 137815 | 062524 | 3, 27, 38 |
| 15 | IGF1R | insulin like growth factor 1 receptor | 140443 | 185087 | 183571 | 31, 12, 18 |
| 15 | ITGA11 | integrin subunit alpha 11 | 137809 | 169018 | 103647 | 6, 34, 43 |
| 15 | ICE2 | interactor pf little elongation complex ELL subunit 2 | 128915 | 182718 | 069667 | 42, 39, 11 |
| 15 | ISG20 | interferon stimulated exonuclease gene 20 | 172183 | 181026 | 157766 | 41, 22, 16 |
| 15 | IL16 | interleukin 16 | 172349 | 156206 | 172345 | 41, 39, 36 |
| 15 | IQGAP1 | IQ mctif containing GTPase activating protein 1 | 140575 | 196391 | 140577 | 24, 23, 30 |
| 15 | IQCH | IQ motif containing H | 103599 | 103591 | 189227 | 38, 42, 23 |
| 15 | IREB2 | iron responsive element binding protein 2 | 136381 | 166426 | 188266 | 4, 1, 39 |
| 15 | ISL2 | ISL LIM homeobox 2 | 159556 | 140374 | 140386 | 27, 5, 14 |
| 15 | IDH2 | isocitrate dehydrogenase (NADP(+)) 2, mitochondrial | 182054 | 140548 | 185033 | 15, 25, 37 |
| 15 | IDH3A | isocitrate dehydrogenase 3 (NAD(+)) alpha | 166411 | 136425 | 103740 | 39, 21, 13 |
| 15 | IVD | ispvaleryl-CpA dehydrogenase | 128928 | 128944 | 140320 | 29, 37, 40 |
| 15 | JMJD7-PLA2G4B | JMJD7-PLA2G4B readthrough | 168970 | 243789 | 243708 | 18, 27, 15 |
| 15 | JMJD7 | jumonji domain containing 7 | 243789 | 279937 | 168970 | |
| 15 | KATNBL1 | katanin regulatory subunit B1 like 1 | 134152 | 182405 | 128463 | |
| 15 | KLHL25 | kelch like family member 25 | 183655 | 170776 | 273540 | 42, 38, 1 |
| 15 | KBTBD13 | kelch repeat and BTB domain containing 13 | 234438 | 103710 | 246922 | 25, 18, 38 |
| 15 | KIAA0101 | KIAA0101 | 166803 | 259316 | 103671 | 7, 4, 34 |
| 15 | KIAA1024 | KIAA1024 | 169330 | 166557 | 136371 | 25, 28, 42 |
| 15 | KIF23 | kinesin family member 23 | 137807 | 137819 | 137818 | 6, 30, 39 |
| 15 | KIF7 | kinesin family member 7 | 166813 | 140534 | 166819 | |
| 15 | KNSTRN | kinetochore localized astrin/SPAG5 binding protein | 128944 | 140323 | 128928 | 6, 38, 12 |
| 15 | KNL1 | kinetochore scaffold 1 | 137812 | 166133 | 279084 | |
| 15 | KLF13 | Kruppel like factor 13 | 169926 | 134160 | 169918 | |
| 15 | LARP6 | La ribonucleoprotein domain family member 6 | 166173 | 137831 | 137821 | 25, 15, 43 |
| 15 | LACTB | lactamase beta | 103642 | 140416 | 185088 | 31, 6, 3 |
| 15 | LCTL | lactase like | 188501 | 174442 | 137834 | 14, 35, 12 |
| 15 | LDHAL6B | lactate dehydrogenase A like 6B | 171989 | 157483 | 157470 | 38, 4, 8 |
| 15 | LMAN1L | lectin, manncse binding 1 like | 140506 | 103653 | 213578 | 33, 34, 36 |
| 15 | LEO1 | LEO1 homolog, Paf1/RNA polymerase II complex component | 166477 | 138594 | 069956 | 30, 2, 13 |
| 15 | LCMT2 | leucine carboxyl methyltransferase 2 | 168806 | 159495 | 168803 | 37, 23, 26 |
| 15 | LINGO1 | leucine rich repeat and Ig domain containing 1 | 169783 | 140382 | 167202 | |
| 15 | LRRC28 | leucine rich repeat containing 28 | 168904 | 103852 | 281635 | |
| 15 | LRRC49 | leucine rich repeat containing 49 | 137821 | 166173 | 129028 | 10, 31, 8 |
| 15 | LRRC57 | leucine rich repeat containing 57 | 180979 | 092531 | 137814 | 1, 22, 30 |
| 15 | LRRK1 | leucine rich repeat kinase 1 | 154237 | 184254 | 131873 | 22, 18, 1 |
| 15 | LTK | leukocyte receptor tyrosine kinase | 062524 | 137825 | 103932 | 16, 38, 24 |
| 15 | LINS1 | lines homolog 1 | 140471 | 154227 | 183475 | |
| 15 | UPC | lipase C, hepatic type | 166035 | 103569 | 137845 | 19, 36, 15 |
| 15 | UNC01314 | Leng intergenic non-protein ceding RNA 1314 | 259417 | 103876 | 172379 | |
| 15 | LYSMD2 | LysM domain containing 2 | 140280 | 104112 | 128872 | 28, 9, 7 |
| 15 | LYSMD4 | LysM domain containing 4 | 183060 | 068305 | 140470 | 13, 26, 22 |
| 15 | LPCAT4 | lysophosphatidylcholine acyltransferase 4 | 176454 | 184507 | 175265 | 16, 20, 26 |
| 15 | LOXL1 | lysyl oxidase like 1 | 129038 | 167139 | 067221 | 6, 45, 32 |
| 15 | MAGEL2 | MAGE family member L2 | 254585 | 281873 | 182636 | 31, 2, 44 |
| 15 | MKRN3 | makorin ring finger protein 3 | 179455 | 174450 | 281873 | 32, |
| 15 | MPI | mannose phosphate isomerase | 178802 | 140497 | 178761 | 13, 9, 2 |
| 15 | MAN2A2 | mannosidase alpha class 2A member 2 | 196547 | 182511 | 184508 | |
| 15 | MAN2C1 | mannosidase alpha class 2C member 1 | 140400 | 140398 | 169375 | |
| 15 | MNS1 | meiosis specific nuclear structural 1 | 138587 | 151575 | 137871 | 38, 4, 32 |
| 15 | MEIS2 | Meis homeobox 2 | 134138 | 186073 | 166069 | 31, 12, 38 |
| 15 | MESDC1 | mesoderm development candidate 1 | 140406 | 117899 | 156206 | 35, 11, 22 |
| 15 | MESDC2 | mesoderm development candidate 2 | 117899 | 103888 | 140406 | 37, 11, 4 |
| 15 | MESP1 | mesoderm posterior bHLH transcription factor 1 | 166823 | 140527 | 188095 | 31, 33, 17 |
| 15 | MESP2 | mesoderm posterior bHLH transcription factor 2 | 188095 | 166823 | 166825 | 10, 15, 14 |
| 15 | MTHFS | methenyltetrahydrofolate synthetase | 136371 | 169330 | 259332 | 28, |
| 15 | MEX3B | mex-3 RNA binding family member B | 183496 | 188869 | 140598 | 38, 12, 8 |
| 15 | MGA | MGA, MAX dimerization protein | 174197 | 092445 | 137802 | 31, 1, 26 |
| 15 | MFAP1 | microfibrillar associated protein 1 | 140259 | 242028 | 092470 | 37, 13, 21 |
| 15 | MAP1A | microtubule associated protein 1A | 166963 | 067369 | 168781 | 26, 8, 31 |
| 15 | MFGE8 | milk fat globule-EGF factor 8 protein | 140545 | 140511 | 140526 | 42, 35, 12 |
| 15 | MTFMT | mitochondrial methionyl-tRNA formyltransferase | 103707 | 090487 | 186198 | 29, 28, 33 |
| 15 | MRPL46 | mitochondrial ribosomal protein L46 | 259494 | 140538 | 181991 | 43, 6, 15 |
| 15 | MRPS11 | mitochondrial ribosomal protein S11 | 181991 | 259494 | 140543 | 41, 3, 37 |
| 15 | MAPK6 | mitogen-activated protein kinase 6 | 069956 | 166477 | 137875 | 34, 7, 42 |
| 15 | MAPKBP1 | mitogen-activated protein kinase binding protein 1 | 137802 | 174197 | 279937 | 4, 38, 40 |
| 15 | MAP2K1 | mitogen-activated protein kinase kinase 1 | 169032 | 075131 | 174446 | 22, 34, 29 |
| 15 | MAP2K5 | mitogen-activated protein kinase kinase 5 | 137764 | 189227 | 188779 | 26, 33, 39 |
| 15 | MORF4L1 | mortality factor 4 like 1 | 185787 | 136378 | 103811 | 12, 11, 31 |
| 15 | MCTP2 | multiple C2 and transmembrane domain containing 2 | 140563 | 182175 | 185551 | 22, 42, 31 |
| 15 | MEGF11 | multiple EGF like domains 11 | 157890 | 103769 | 166938 | 38, 8, 14 |
| 15 | MYEF2 | myelin expression factor 2 | 104177 | 188467 | 233932 | 38, |
| 15 | MYZAP | myocardial zonula adherens protein | 263155 | 137878 | 255529 | |
| 15 | MEF2A | myocyte enhancer factor 2 A | 068305 | 281635 | 183060 | 43, 45, 29 |
| 15 | MYO1E | myosin IE | 157483 | 157456 | 171989 | 21, 3, 22 |
| 15 | MYO9A | myosin IXA | 066933 | 278570 | 166192 | 9, 1, 32 |
| 15 | MYO5A | myosin VA | 197535 | 128833 | 280817 | 26, 8, 35 |
| 15 | MYO5C | myosin VC | 128833 | 069966 | 197535 | 42, 37, 13 |
| 15 | MTMR10 | myotubularin related protein 10 | 166912 | 198690 | 134160 | 3, 11, 22 |
| 15 | NDUFAF1 | NADH: ubiquinone oxidoreductase complex assembly factor 1 | 137806 | 137804 | 137815 | 33, 38, 17 |
| 15 | NOX5 | NADPH oxidase 5 | 255346 | 258484 | 138604 | 38, 36, 31 |
| 15 | NANOGP8 | Nanog homeobox pseudogene 8 | 255192 | 198146 | 134146 | |
| 15 | NDN | necdin, MAGE family member | 182636 | 254585 | 185823 | 11, 34, 32 |
| 15 | NEIL1 | nei like DNA glycosylase 1 | 140398 | 140365 | 140400 | 39, 22, 29 |
| 15 | NEO1 | neogenin 1 | 067141 | 159322 | 138622 | 29, 11, 16 |
| 15 | NGRN | neugrin, neurite outgrowth associated | 182768 | 261147 | 275674 | 28, 18, 9 |
| 15 | NEDD4 | neural precursor cell expressed, developmentally down-regulated 4, | 069869 | 166450 | 181827 | 4, 45, 19 |
| | | E3 ubiquitin protein ligase | | | | |
| 15 | NRG4 | neuregulin 4 | 169752 | 167196 | 169758 | |
| 15 | NMB | neuromedin B | 197696 | 177082 | 140612 | 40, 13, 17 |
| 15 | NPTN | neuroplastin | 156642 | 183324 | 103855 | 31, 13, 26 |
| 15 | NTRK3 | neurotrophic receptor tyrosine kinase 3 | 140538 | 166748 | 259494 | |
| 15 | NIPA1 | non imprinted in Prader-Willi/Angelman syndrome 1 | 170113 | 277865 | 140157 | 9, 36, 8 |
| 15 | NIPA2 | non imprinted in Prader-Willi/Angelman syndrome 2 | 140157 | 170113 | 273749 | 11, 2, 28 |
| 15 | NOP10 | NOP10 ribonucleoprotein | 182117 | 140199 | 184507 | 15, 7, 2 |
| 15 | NSMCE3 | NSE3 homolog, SMC5-SMC6 complex component | 185115 | 104059 | 104067 | 29, 2, 13 |
| 15 | NPAP1 | nuclear pore associated protein 1 | 185823 | 182636 | 128739 | 27, 6, 38 |
| 15 | NR2E3 | nuclear receptor subfamily 2 group E member 3 | 278570 | 225362 | 066933 | 25, 14, 27 |
| 15 | NR2F2 | nuclear receptor subfamily 2 group F member 2 | 185551 | 140563 | 185594 | 11, 12, 1 |
| 15 | NUSAP1 | nucleolar and spindle associated protein 1 | 137804 | 104147 | 137806 | 19, 13, 39 |
| 15 | NUTM1 | NUT midline carcinoma family member 1 | 184507 | 182117 | 176454 | 38, 8, |
| 15 | OCA2 | OCA2 melanosomal transmembrane protein | 104044 | 182256 | 128731 | 35, 32, 38 |
| 15 | OR4F15 | olfactory receptor family 4 subfamily F member 15 | 182854 | 184140 | 177693 | |
| 15 | OR4F4 | olfactory receptor family 4 subfamily F member 4 | 177693 | 182854 | 279084 | |
| 15 | OR4F6 | olfactory receptor family 4 subfamily F member 6 | 184140 | 185418 | 182854 | |
| 15 | OR4M2 | olfactory receptor family 4 subfamily M member 2 | 274102 | 233917 | 183706 | |
| 15 | OR4N4 | olfactory receptor family 4 subfamily N member 4 | 183706 | 274102 | 270505 | 38, 18, 8 |
| 15 | ONECUT1 | one cut homeobox 1 | 169856 | 047346 | 166415 | 6, 14, 20 |
| 15 | OIP5 | Opa interacting protein 5 | 104147 | 187446 | 137804 | 22, 37, 4 |
| 15 | OAZ2 | ornithine decarboxylase antizyme 2 | 180304 | 180357 | 166831 | 43, 41, 9 |
| 15 | OTUD7A | OTU deubiquitinase 7A | 169918 | 169926 | 175344 | 38, 8, 17 |
| 15 | ODF3L1 | outer dense fiber of sperm tails 3 like 1 | 182950 | 173546 | 140367 | 38, 20, 8 |
| 15 | PAK6 | p21 (RAC1) activated kinase 6 | 137843 | 259288 | 137841 | 38, 8, 42 |
| 15 | PATL2 | PAT1 homolog 2 | 229474 | 104133 | 166710 | 39, 5, 22 |
| 15 | PPIB | peptidylprolyl isomerase B | 166794 | 157734 | 169118 | 4, 37, 2 |
| 15 | PLIN1 | perilipin 1 | 166819 | 166813 | 166821 | 1, 26, 15 |
| 15 | PEX11A | peroxisomal biogenesis factor 11 alpha | 166821 | 166819 | 140527 | 1, 23, 17 |
| 15 | PIGB | phosphatidylinositol glycan anchor biosynthesis class B | 069943 | 225973 | 260916 | 13, 43, 4 |
| 15 | PDE8A | phosphodiesterase 8A | 073417 | 156222 | 170776 | 4, 18, 11 |
| 15 | PLA2G4B | phospholipase A2 group IVB | 243708 | 168970 | 137877 | |
| 15 | PLA2G4D | phospholipase A2 group IVD | 159337 | 188089 | 168907 | 33, 35, 8 |
| 15 | PLA2G4E | phospholipase A2 group IVE | 188089 | 103966 | 159337 | 4, 24, 39 |
| 15 | PLA2G4F | phospholipase A2 group IVF | 168907 | 159337 | 166887 | 24, 30, 42 |
| 15 | PLCB2 | phospholipase C beta 2 | 137841 | 137843 | 230778 | 7, 5, 22 |
| 15 | PPCDC | phosphopantothenoylcysteine decarboxylase | 138621 | 198794 | 167173 | |
| 15 | PIF1 | PIF1 5′-to-3′ DNA helicase | 140451 | 166831 | 241839 | 22, 27, 12 |
| 15 | PIGBOS1 | PIGB opposite strand 1 | 225973 | 069974 | 069943 | |
| 15 | PGBD4 | piggyBac transposable element derived 4 | 182405 | 134153 | 134152 | 10, 44, 38 |
| 15 | PLEKHO2 | pleckstrin homology domain containing O2 | 241839 | 140451 | 166839 | 36, 1, 35 |
| 15 | PARP16 | poly(ADP-ribose) polymerase family member 16 | 138617 | 138615 | 174498 | 1, 22, 23 |
| 15 | PARP6 | poly(ADP-ribose) polymerase family member 6 | 137817 | 067225 | 273025 | 18, 1, 38 |
| 15 | POTEB | POTE ankyrin domain family member B | 233917 | 278263 | 274102 | |
| 15 | POTEB2 | POTE ankyrin domain family member B2 | 230031 | 277322 | 270824 | |
| 15 | POTEB3 | POTE ankyrin domain family member B3 | 278522 | 270185 | 182974 | |
| 15 | PAQR5 | progestin and adipoQ receptor family member 5 | 137819 | 138604 | 137807 | 31, 29, 9 |
| 15 | PDCD7 | programmed cell death 7 | 090470 | 246922 | 166855 | 31, 18, 4 |
| 15 | PHGR1 | proline, histidine and glycine rich 1 | 233041 | 188549 | 140323 | 16, 37, |
| 15 | PSTPIP1 | proline-serine-threonine phosphatase interacting protein 1 | 140368 | 117906 | 140391 | 36, 5, 39 |
| 15 | PML | promyelocytic leukemia | 140464 | 067221 | 159289 | 34, 40, 22 |
| 15 | PCSK6 | proprotein convertase subtilisin/kexin type 6 | 140479 | 131876 | 184277 | 19, 9, 38 |
| 15 | PSMA4 | proteasome subunit alpha 4 | 041357 | 188266 | 169684 | 4, 34, 7 |
| 15 | PDIA3 | protein disulfide isomerase family A member 3 | 167004 | 223572 | 128886 | 13, 18, 12 |
| 15 | PIAS1 | protein inhibitor of activated STAT 1 | 033800 | 188779 | 260007 | 11, 28, 7 |
| 15 | PPP1R14D | protein phosphatase 1 regulatory inhibitor subunit 14D | 166143 | 166140 | 166145 | 16, 17, 44 |
| 15 | PRC1 | protein regulator of cytokinesis 1 | 198901 | 166965 | 184056 | 19, 39, 18 |
| 15 | PTPN9 | protein tyrosine phosphatase, non-receptor type 9 | 169410 | 169375 | 169371 | 34, 31, 20 |
| 15 | PRTG | protogenin | 166450 | 171016 | 069869 | 12, 38, 18 |
| 15 | PEAK1 | pseudopodium enriched atypical kinase 1 | 173517 | 140391 | 140382 | 11, 7, 15 |
| 15 | PYGO1 | pygopus family PHD finger 1 | 171016 | 256061 | 166450 | 40, 9, 12 |
| 15 | PGPEP1L | pyroglutamyl-peptidase l-like | 183571 | 140443 | 182253 | 38, |
| 15 | PKM | pyruvate kinase, muscle | 067225 | 175318 | 137817 | 21, 34, 35 |
| 15 | RAB11A | RAB11 A, member RAS oncogene family | 103769 | 174485 | 157890 | 4, 45, 11 |
| 15 | RAB27A | RAB27A, member RAS oncogene family | 069974 | 137876 | 225973 | 34, 35, 7 |
| 15 | RAB8B | RAB8B, member RAS oncogene family | 166128 | 185088 | 138613 | 45, 25, 7 |
| 15 | RAD51 | RAD51 recombinase | 051180 | 279084 | 137824 | 7, 2, 39 |
| 15 | RORA | RAR related orphan receptor A | 069667 | 128915 | 129003 | |
| 15 | RASGRP1 | RAS guanyl releasing protein 1 | 172575 | 171262 | 175779 | 22, 36, 39 |
| 15 | RHOV | ras homolog family member V | 104140 | 166145 | 104142 | 9, 4, 32 |
| 15 | RASL12 | RAS like family 12 | 103710 | 186198 | 234438 | 26, 33, 20 |
| 15 | RASGRF1 | Ras protein specific guanine nucleotide releasing factor 1 | 058335 | 103811 | 235711 | 26, 5, 8 |
| 15 | RCCD1 | RCC1 domain containing 1 | 166965 | 140553 | 198901 | 9, 29, 1 |
| 15 | REC114 | REC114 meiotic recombination protein | 183324 | 138622 | 156642 | |
| 15 | RMDN3 | regulator of microtubule dynamics 3 | 137824 | 051180 | 137880 | 34, 25, 27 |
| 15 | RFX7 | regulatory factor X7 | 181827 | 069869 | 151575 | 34, 26, 11 |
| 15 | RGMA | repulsive guidance molecule family member a | 182175 | 173575 | 140563 | 24, 25, 38 |
| 15 | RCN2 | reticulocalbin 2 | 117906 | 140386 | 140368 | 11, 4, 29 |
| 15 | RLBP1 | retinaldehyde binding protein 1 | 140522 | 140526 | 140525 | 14, 10, 35 |
| 15 | RHCG | Rh family C glycoprotein | 140519 | 140521 | 140534 | 13, 24, 18 |
| 15 | ARHGAP11A | Rho GTPase activating protein 11A | 198826 | 232653 | 166922 | |
| 15 | ARHGAP11B | Rho GTPase activating protein 11B | 187951 | 261794 | 198690 | |
| 15 | RPP25 | ribonuclease P/MRP subunit p25 | 178718 | 178741 | 198794 | 29, 9, 26 |
| 15 | RSL24D1 | ribosomal L24 domain containing 1 | 137876 | 137766 | 069974 | 34, 7, 4 |
| 15 | RPL4 | ribosomal protein L4 | 174444 | 174446 | 174442 | |
| 15 | RPLP1 | ribosomal protein lateral stalk subunit P1 | 137818 | 137807 | 140332 | 43, 7, 25 |
| 15 | RPS17 | ribosomal protein S17 | 182774 | 197978 | 260836 | |
| 15 | RPS27L | ribosomal protein S27 like | 185088 | 103642 | 166128 | 29, 24, 7 |
| 15 | RNF111 | ring finger protein 111 | 157450 | 128923 | 137776 | 11, 19, 40 |
| 15 | RBPMS2 | RNA binding protein with multiple splicing 2 | 166831 | 180304 | 140451 | 15, 12, 44 |
| 15 | RPAP1 | RNA polymerase II associated protein 1 | 103932 | 062524 | 092445 | 43, 38, 21 |
| 15 | POLR2M | RNA polymerase II subunit M | 255529 | 263155 | 128918 | |
| 15 | RPUSD2 | RNA pseudouridylate synthase domain containing 2 | 166133 | 128891 | 137812 | 9, 21, 45 |
| 15 | RTF1 | RTF1 homolog, Paf1/RNA polymerase II complex component | 137815 | 137806 | 137825 | 29, 25, 22 |
| 15 | RYR3 | ryanodine receptor 3 | 198838 | 248905 | 169857 | 10, 36, 2 |
| 15 | SLTM | SAFB like transcription modulator | 137776 | 157450 | 157456 | 24, 26, 11 |
| 15 | SEC11A | SEC11 homolog A, signal peptidase complex subunit | 140612 | 197696 | 166716 | 18, 26, 31 |
| 15 | SECISBP2L | SECIS binding protein 2 like | 138593 | 255302 | 166200 | 1, 26, 22 |
| 15 | SCG3 | secretogranin III | 104112 | 104093 | 140280 | 28, 8, 31 |
| 15 | SCG5 | secretogranin V | 166922 | 198826 | 166923 | 28, 31, 29 |
| 15 | SCAMP2 | secretary carrier membrane protein 2 | 140497 | 140474 | 178802 | 36, 21, 6 |
| 15 | SCAMP5 | secretory carrier membrane protein 5 | 198794 | 178718 | 138621 | |
| 15 | SELENOS | selenoprotein S | 131871 | 131873 | 131876 | |
| 15 | SEMA4B | semaphorin 4B | 185033 | 182054 | 185043 | |
| 15 | SEMA6D | semaphprin 6D | 137872 | 259752 | 188467 | |
| 15 | SEMA7A | semaphprin 7A (John Milton Hagen blood group) | 138623 | 140459 | 138629 | 32, 8, 2 |
| 15 | SERINC4 | serine incorporator 4 | 184716 | 279808 | 242028 | 3, 31, 25 |
| 15 | SPINT1 | serine peptidase inhibitor, Kunitz type 1 | 166145 | 166143 | 104140 | 32, 16, 29 |
| 15 | SH2D7 | SH2 domain containing 7 | 183476 | 167202 | 136425 | 32, |
| 15 | SH3GL3 | SH3 domain containing GRB2 like 3, endophilin A3 | 140600 | 169594 | 156218 | 26, 8, 38 |
| 15 | SHC4 | SHC adaptor protein 4 | 185634 | 103995 | 255302 | 26, 11, 38 |
| 15 | SPPL2A | signal peptide peptidase like 2A | 138600 | 092439 | 081014 | 10, 11, 29 |
| 15 | SRP14 | signal recognition particle 14 | 140319 | 128829 | 104081 | 43, 41, 34 |
| 15 | SIN3A | SIN3 transcription regulator family member A | 169375 | 140400 | 169410 | 34, 7, 24 |
| 15 | SKOR1 | SKI family transcriptional corepressor 1 | 188779 | 137764 | 033800 | 22, 20, 38 |
| 15 | SMAD3 | SMAD family member 3 | 166949 | 137834 | 103591 | 34, 15, 1 |
| 15 | SMAD6 | SMAD family member 6 | 137834 | 188501 | 166949 | 27, 29, 20 |
| 15 | SERF2 | small EDRK-rich factor 2 | 140264 | 262560 | 279808 | |
| 15 | SNRPA1 | small nuclear ribonucleoprotein polypeptide A′ | 131876 | 131871 | 140479 | 18, 4, 31 |
| 15 | SNRPN | small nuclear ribonucleoprotein polypeptide N | 128739 | 185823 | 273173 | |
| 15 | SNAPC5 | small nuclear RNA activating complex polypeptide 5 | 174446 | 169032 | 174444 | 29, 43, 21 |
| 15 | SNURF | SNRPN upstream reading frame | 273173 | 128739 | 214265 | |
| 15 | SNUPN | snurportin 1 | 169371 | 169410 | 177971 | 40, 27, 26 |
| 15 | SLC12A1 | splute carrier family 12 member 1 | 074803 | 233932 | 128951 | 17, 29, 25 |
| 15 | SLC12A6 | solute carrier family 12 member 6 | 140199 | 128463 | 182117 | 38, 6, 9 |
| 15 | SLC24A1 | solute carrier family 24 member 1 | 074621 | 138614 | 174485 | 14, 29, 4 |
| 15 | SLC24A5 | solute carrier family 24 member 5 | 188467 | 137872 | 104177 | 35, 45, 28 |
| 15 | SLC27A2 | solute carrier family 27 member 2 | 140284 | 104043 | 140287 | 17, 13, 32 |
| 15 | SLC28A1 | solute carrier family 28 member 1 | 156222 | 136383 | 073417 | 17, 22, 19 |
| 15 | SLC28A2 | solute carrier family 28 member 2 | 137860 | 138606 | 171766 | 17, 10, 16 |
| 15 | SLC30A4 | solute carrier family 30 member 4 | 104154 | 166920 | 104164 | 22, 21, 38 |
| 15 | SLC51B | solute carrier family 51 beta subunit | 186198 | 103707 | 103710 | 17, 16, 38 |
| 15 | SLCO3A1 | solute carrier organic anion transporter family member 3A1 | 176463 | 185518 | 140557 | 38, 26, 22 |
| 15 | SORD | sorbitol dehydrogenase | 140263 | 167014 | 140279 | 33, 40, 26 |
| 15 | SNX1 | sorting nexin 1 | 028528 | 166797 | 157734 | 40, 23, 1 |
| 15 | SNX22 | sorting nexin 22 | 157734 | 028528 | 166794 | 16, 40, 18 |
| 15 | SNX33 | sorting nexin 33 | 173548 | 177971 | 173546 | 1, 4, 34 |
| 15 | SPG11 | spastic paraplegia 11 (autosomal recessive) | 104133 | 104131 | 229474 | 30, 2, 40 |
| 15 | SPG21 | spastic paraplegia 21 (autosomal recessive, Mast syndrome) | 090487 | 166839 | 103707 | 40, 18, 31 |
| 15 | SPTBN5 | spectrin beta, non-erythrocytic 5 | 137877 | 243708 | 103966 | 14, 15, 42 |
| 15 | SPESP1 | sperm equatorial segment protein 1 | 258484 | 140350 | 255346 | |
| 15 | SPATA5L1 | spermatogenesis associated 5 like 1 | 171763 | 171766 | 166920 | 9, 24, 4 |
| 15 | SPATA8 | spermatogenesis associated 8 | 185594 | 185551 | 140450 | 38, 8, |
| 15 | SCAPER | S-phase cyclin A associated protein in the ER | 140386 | 159556 | 117906 | 6, 10, 14 |
| 15 | SPRED1 | sprouty related EVH1 domain containing 1 | 166068 | 166069 | 171262 | 11, 42, 30 |
| 15 | SHF | Src homology 2 domain containing F | 138606 | 137857 | 137860 | 26, 14, 4 |
| 15 | ST20-MTHFS | ST20-MTHFS readthrough | 259332 | 136371 | 180953 | |
| 15 | ST8SIA2 | ST8 alpha-N-acetyl-neuraminide alpha-2,8-sialyltransferase 2 | 140557 | 176463 | 185442 | 18, 8, 14 |
| 15 | SAXO2 | stabilizer of axonemal microtubules 2 | 188659 | 140598 | 278662 | 42, 40, 22 |
| 15 | STARD5 | StAR related lipid transfer domain containing 5 | 172345 | 172349 | 188869 | 34, 29, 32 |
| 15 | STARD9 | StAR related lipid transfer domain containing 9 | 159433 | 137814 | 140326 | 1, 18, 26 |
| 15 | STRC | stereocilin | 242866 | 237289 | 166762 | 37, 39, 8 |
| 15 | STRA6 | stimulated by retinoic acid 6 | 137868 | 129009 | 140481 | 32, 40, 38 |
| 15 | STOML1 | stomatin like 1 | 067221 | 129038 | 140464 | 4, 8, 42 |
| 15 | SQRDL | sulfide quinone reductase-like (yeast) | 137767 | 260170 | 259752 | 16, 5, 13 |
| 15 | SENP8 | SUMO/sentrin peptidase family member, NEDD8 specific | 166192 | 066933 | 175318 | 9, 30, 2 |
| 15 | ST20 | suppressor of tumorigenicity 20 | 180953 | 259332 | 140379 | 10, 22, 15 |
| 15 | SV2B | synaptic vesicle glycoprotein 2B | 185518 | 184056 | 176463 | 26, 14, 8 |
| 15 | SNAP23 | synaptosome associated protein 23 | 092531 | 103994 | 180979 | 45, 5, 36 |
| 15 | SYNM | synemin | 182253 | 183571 | 103852 | 40, 26, 16 |
| 15 | TLN2 | talin 2 | 171914 | 205502 | 140416 | 14, 15, 3 |
| 15 | TTBK2 | tau tubulin kinase 2 | 128881 | 140326 | 159459 | 9, 22, 38 |
| 15 | TBC1D21 | TBC1 domain family member 21 | 167139 | 205363 | 129038 | 38, 8, |
| 15 | TBC1D2B | TBC1 domain family member 2B | 167202 | 169783 | 183476 | 39, 24, 4 |
| 15 | TERB2 | telomere repeat binding bouquet formation protein 2 | 167014 | 185880 | 140263 | 38, |
| 15 | TEX9 | testis expressed 9 | 151575 | 181827 | 138587 | |
| 15 | TSPAN3 | tetraspanin 3 | 140391 | 140368 | 173517 | 16, 8, 35 |
| 15 | TTC23 | tetratricopeptide repeat domain 23 | 103852 | 182253 | 168904 | 24, 18, 1 |
| 15 | THAP10 | THAP domain containing 10 | 129028 | 137821 | 187720 | 4, 13, 8 |
| 15 | TARSL2 | threonyl-tRNA synthetase like 2 | 185418 | 184277 | 184140 | 31, 9, 6 |
| 15 | THBS1 | thrombospondin 1 | 137801 | 175779 | 150667 | 13, 45, 6 |
| 15 | THSD4 | thrombospondin type 1 domain containing 4 | 187720 | 129028 | 225362 | 42, 26, 23 |
| 15 | TRIP4 | thyroid hormone receptor interactor 4 | 103671 | 166803 | 180357 | 13, 10, 11 |
| 15 | TJP1 | tight junction protein 1 | 104067 | 185115 | 179938 | 1, 24, 26 |
| 15 | TIPIN | TIMELESS interacting protein | 075131 | 166938 | 169032 | 4, 43, 2 |
| 15 | TM2D3 | TM2 domain containing 3 | 184277 | 140479 | 185418 | |
| 15 | TNFAIP8L3 | TNF alpha induced protein 8 like 3 | 183578 | 081014 | 137869 | 34, 24, 38 |
| 15 | TICRR | TOPBP1 interacting checkpoint and replication regulator | 140534 | 140519 | 166813 | 34, 3, 18 |
| 15 | TCF12 | transcription factor 12 | 140262 | 137871 | 128849 | |
| 15 | TLE3 | transducin like enhancer of split 3 | 140332 | 137818 | 137831 | 22, 40, 9 |
| 15 | TGM5 | transglutaminase 5 | 104055 | 166946 | 159495 | 24, 5, 39 |
| 15 | TGM7 | transglutaminase 7 | 159495 | 104055 | 168806 | 15, 38, |
| 15 | TRPM1 | transient receptor potential cation channel subfamily M member 1 | 134160 | 166912 | 169926 | 35, 14, 38 |
| 15 | TRPM7 | transient receptor potential cation channel subfamily M member 7 | 092439 | 170236 | 138600 | 13, 31, 30 |
| 15 | TM6SF1 | transmembrane 6 superfamily member 1 | 136404 | 064726 | 166503 | 45, 5, 37 |
| 15 | TMCO5A | transmembrane and coiled-coil domains 5A | 166069 | 134138 | 166068 | 38, 30, 8 |
| 15 | TMC3 | transmembrane channel like 3 | 188869 | 172345 | 183496 | 29, 14, 20 |
| 15 | TMED3 | transmembrane p24 trafficking protein 3 | 166557 | 235711 | 169330 | 9, 35, 28 |
| 15 | TMEM202 | transmembrane protein 202 | 187806 | 213614 | 166233 | 38, 10, |
| 15 | TMEM266 | transmembrane protein 266 | 169758 | 169752 | 140374 | 32, 36, 10 |
| 15 | TMEM62 | transmembrane protein 62 | 137842 | 166947 | 166946 | 22, 16, 1 |
| 15 | TMEM87A | transmembrane protein 87A | 103978 | 166887 | 214013 | 13, 30, 42 |
| 15 | TRIM69 | tripartite motif containing 69 | 185880 | 166710 | 167014 | 11, 38, 37 |
| 15 | TMOD2 | tropomodulin 2 | 128872 | 140280 | 138594 | |
| 15 | TMOD3 | tropomodulin 3 | 138594 | 128872 | 166477 | 37, 38, 20 |
| 15 | TPM1 | tropomyosin 1 (alpha) | 140416 | 171914 | 103642 | |
| 15 | TUBGCP4 | tubulin gamma complex associated protein 4 | 137822 | 140265 | 067369 | 26, 39, 31 |
| 15 | TUBGCP5 | tubulin gamma complex associated protein 5 | 275835 | 273749 | 273976 | 42, 9, 25 |
| 15 | TP53BP1 | tumor protein p53 binding protein 1 | 067369 | 137822 | 166963 | 23, 18, 1 |
| 15 | TYRO3 | TYRO3 protein tyrosine kinase | 092445 | 103932 | 174197 | 26, 27, 1 |
| 15 | UBAP1L | ubiquitin associated protein 1 like | 246922 | 234438 | 090470 | 1, 14, 35 |
| 15 | UBE2Q2 | ubiquitin conjugating enzyme E2 Q2 | 140367 | 182950 | 167196 | 34, 29, 44 |
| 15 | UBE2Q2L | ubiquitin conjugating enzyme E2 Q2 like | 259511 | 156218 | 184206 | |
| 15 | UBL7 | ubiquitin like 7 | 138629 | 138623 | 179361 | 35, 21, 9 |
| 15 | UBR1 | ubiquitin protein ligase E3 component n-recognin 1 | 159459 | 128881 | 166947 | 1, 26, 33 |
| 15 | UBE3A | ubiquitin protein ligase E3A | 114062 | 279735 | 206190 | 22, 38, |
| 15 | USP3 | ubiquitin specific peptidase 3 | 140455 | 074410 | 197361 | 1, 11, 39 |
| 15 | USP50 | ubiquitin specific peptidase 50 | 170236 | 138592 | 092439 | 38, 5, |
| 15 | USP8 | ubiquitin specific peptidase 8 | 138592 | 104064 | 170236 | 11, 34, 4 |
| 15 | UNC13C | unc-13 homolog C | 137766 | 166415 | 137876 | 35, 14, 8 |
| 15 | UNC45A | unc-45 myosin chaperone A | 140553 | 184508 | 166965 | 43, 24, 39 |
| 15 | ULK3 | unc-51 like kinase 3 | 140474 | 213578 | 140497 | 31, 2, 9 |
| 15 | UACA | uveal autoantigen with coiled-coil domains and ankyrin repeats | 137831 | 140332 | 166173 | 1, 45, 43 |
| 15 | VPS13C | vacuolar protein sorting 13 homolog C | 129003 | 069667 | 198535 | 22, 42, 26 |
| 15 | VWA9 | Von Willebrand factor A domain containing 9 | 138614 | 074696 | 074621 | 45, 9, 17 |
| 15 | VPS18 | VPS18, CORVET/HOPS core subunit | 104142 | 104140 | 128917 | 21, 7, 22 |
| 15 | VPS33B | VPS33B, late endosome and lysosome associated | 184056 | 198901 | 185518 | 9, 45, 21 |
| 15 | VPS39 | VPS39, HOPS complex subunit | 166887 | 168907 | 103978 | 4, 41, 22 |
| 15 | WHAMM | WAS protein homolog associated with actin, golgi membranes and microtubules | 156232 | 186628 | 103942 | 4, 39, 6 |
| 15 | WDR61 | WD repeat domain 61 | 140395 | 140403 | 166426 | 34, 43, 38 |
| 15 | WDR72 | WD repeat domain 72 | 166415 | 169856 | 137766 | |
| 15 | WDR73 | WD repeat domain 73 | 177082 | 176371 | 197696 | 36, 41, 44 |
| 15 | WDR76 | WD repeat domain 76 | 092470 | 140259 | 171877 | 13, 22, 9 |
| 15 | WDR93 | WD repeat domain 93 | 140527 | 166821 | 166823 | 10, 38, 30 |
| 15 | ZFAND6 | zinc finger AN 1-type containing 6 | 086666 | 140379 | 103876 | |
| 15 | ZSCAN2 | zinc finger and SCAN domain containing 2 | 176371 | 184206 | 177082 | 26, 42, 39 |
| 15 | ZSCAN29 | zinc finger and SCAN domain containing 29 | 140265 | 168803 | 137822 | 21, 40, 22 |
| 15 | ZFYVE19 | zinc finger FYVE-type containing 19 | 166140 | 188277 | 166143 | 9, 34, 27 |
| 15 | ZNF106 | zinc finger protein 106 | 103994 | 092529 | 092531 | 25, 1, 15 |
| 15 | ZNF280D | zinc finger protein 280D | 137871 | 138587 | 140262 | |
| 15 | ZNF592 | zinc finger protein 592 | 166716 | 140612 | 136383 | 41, 22, 40 |
| 15 | ZNF609 | zinc finger protein 609 | 180357 | 103671 | 180304 | |
| 15 | ZNF710 | zinc finger protein 710 | 140548 | 242498 | 182054 | 28, 16, 26 |
| 15 | ZNF770 | zinc finger protein 770 | 198146 | 021776 | 255192 | 31, 17, 4 |
| 15 | ZNF774 | zinc finger protein 774 | 196391 | 275674 | 140575 | 29, 2, 24 |
| 15 | ZWILCH | zwilch kinetochore protein | 174442 | 174444 | 188501 | 38, 35, 43 |
| 16 | DECR2 | 2,4-dienoyl-CoA reductase 2 | 242612 | 103202 | 090565 | 13, 16, |
| 16 | OGFOD1 | 2-oxoglutarate and iron dependent oxygenase domain containing 1 | 087263 | 167005 | 125124 | 36, 7, 25 |
| 16 | PDPK1 | 3-phosphoinositide dependent protein kinase 1 | 140992 | 205923 | 167977 | |
| 16 | ABAT | 4-aminobutyrate aminotransferase | 183044 | 067365 | 184857 | |
| 16 | ACD | ACD, shelterin complex subunit and telomerase recruitment factor | 102977 | 159753 | 102981 | 22, 34, 9 |
| 16 | ATF7IP2 | activating transcription factor 7 interacting protein 2 | 166669 | 183454 | 213853 | |
| 16 | ACSF3 | acyl-CoA synthetase family member 3 | 176715 | 129993 | 129910 | |
| 16 | ACSM1 | acyl-CoA synthetase medium-chain family member 1 | 166743 | 005187 | 066654 | 4, 40, 33 |
| 16 | ACSM2A | acyl-CoA synthetase medium-chain family member 2A | 183747 | 183549 | 066813 | 17, 19, 23 |
| 16 | ACSM2B | acyl-CoA synthetase medium-chain family member 2B | 066813 | 183747 | 005187 | |
| 16 | ACSM3 | acyl-CoA synthetase medium-chain family member 3 | 005187 | 066813 | 166743 | 7, 17, 4 |
| 16 | ACSM5 | acyl-CoA synthetase medium-chain family member 5 | 183549 | 169340 | 183747 | 19, 17, 2 |
| 16 | ADAMTS18 | ADAM metallopeptidase with thrombospondin type 1 motif 18 | 140873 | 205078 | 140876 | 14, 20, 32 |
| 16 | AP1G1 | adaptor related protein complex 1 gamma 1 subunit | 166747 | 040199 | 224470 | |
| 16 | APRT | adenine phosphoribosyltransferase | 198931 | 167513 | 141012 | 7, 35, 33 |
| 16 | ADAD2 | adenosine deaminase domain containing 2 | 140955 | 103168 | 168418 | 38, 14, 40 |
| 16 | ADAT1 | adenosine deaminase, tRNA specific 1 | 065457 | 034713 | 065427 | 43, 24, 9 |
| 16 | ADCY7 | adenylate cyclase 7 | 121281 | 121274 | 166164 | 22, 7, 32 |
| 16 | ADCY9 | adenylate cyclase 9 | 162104 | 005339 | 185739 | 26, 11, 13 |
| 16 | ADGRG1 | adhesion G protein-coupled receptor G1 | 205336 | 159618 | 182885 | 40, 42, 38 |
| 16 | ADGRG3 | adhesion G protein-coupled receptor G3 | 182885 | 205336 | 159625 | 7, 1, 36 |
| 16 | ADGRG5 | adhesipn G prptein-cpupled receptor G5 | 159618 | 135736 | 205336 | 22, 7, 41 |
| 16 | ARL2BP | ADP ribosylation factor like GTPase 2 binding protein | 102931 | 159579 | 102934 | 43, 2, 1 |
| 16 | ARL6IP1 | ADP ribosylation factor like GTPase 6 interacting protein 1 | 170540 | 260342 | 157106 | 8, 4, 45 |
| 16 | AGRP | agouti related neuropeptide | 159723 | 159720 | 039523 | 10, 17, 22 |
| 16 | AKTIP | AKT interacting protein | 166971 | 103479 | 103494 | 45, 11, 39 |
| 16 | AARS | alanyl-tRNA synthetase | 090861 | 223496 | 157349 | 21, 9, 40 |
| 16 | ALDOA | aldolase, fructose-bisphosphate A | 149925 | 149926 | 149923 | 30, 18, 25 |
| 16 | ALG1 | ALG1, chitobiosyldiphosphodolichol beta-mannosyltransferase | 033011 | 103174 | 153446 | 43, 2, 9 |
| 16 | AHSP | alpha hemoglobin stabilizing protein | 169877 | 140688 | 197302 | 5, 36, 7 |
| 16 | AMDHD2 | amidohydrolase domain containing 2 | 162066 | 259784 | 205923 | 29, 37, 14 |
| 16 | ANKS3 | ankyrin repeat and sterile alpha motif domain containing 3 | 168096 | 168101 | 166246 | 13, 34, 32 |
| 16 | ANKS4B | ankyrin repeat and sterile alpha motif domain containing 4B | 175311 | 103310 | 103316 | 16, 19, 17 |
| 16 | ANKRD11 | ankyrin repeat domain 11 | 167522 | 170100 | 197912 | |
| 16 | APOBR | apolipoprotein B receptor | 184730 | 283220 | 197272 | 5, 4, 2 |
| 16 | AQP8 | aquaporin 8 | 103375 | 205629 | 155592 | 10, 16, 28 |
| 16 | ARMC5 | armadillo repeat containing 5 | 140691 | 176723 | 140682 | 7, 26, 39 |
| 16 | ASPHD1 | aspartate beta-hydroxylase domain containing 1 | 174939 | 174938 | 174943 | 26, 28, 35 |
| 16 | ATXN1L | ataxin 1 like | 224470 | 166747 | 182149 | 17, 1, 14 |
| 16 | ATXN2L | ataxin 2 like | 168488 | 196993 | 178952 | 20, |
| 16 | ATMIN | ATM interactor | 166454 | 166451 | 166455 | 29, 22, 44 |
| 16 | ABCA3 | ATP binding cassette subfamily A member 3 | 167972 | 279473 | 162063 | 14, 1, 3 |
| 16 | ABCC1 | ATP binding cassette subfamily C member 1 | 103222 | 133393 | 091262 | 7, 30, 9 |
| 16 | ABCC11 | ATP binding cassette subfamily C member 11 | 121270 | 140798 | 102910 | 23, 38, 33 |
| 16 | ABCC12 | ATP binding cassette subfamily C member 12 | 140798 | 102893 | 121270 | 7, 38, 19 |
| 16 | ABCC6 | ATP binding cassette subfamily C member 6 | 091262 | 103222 | 103226 | 17, 3, 19 |
| 16 | ATP6V0C | ATPase H+ transporting V0 subunit c | 185883 | 260272 | 259784 | 37, 26, 27 |
| 16 | ATP6V0D1 | ATPase H+ transporting V0 subunit d1 | 159720 | 176387 | 159723 | 43, 18, 41 |
| 16 | ATP2A1 | ATPase sarcoplasmic/endoplasmic reticulum Ca2+ transporting 1 | 196296 | 178188 | 177548 | 25, 18, 24 |
| 16 | ATP2C2 | ATPase secretary pathway Ca2+ transporting 2 | 064270 | 103175 | 140950 | |
| 16 | AMFR | autocrine motility factor receptor | 159461 | 087258 | 167005 | 5, 9, 30 |
| 16 | AXIN1 | axin 1 | 103126 | 185615 | 086504 | 9, 40, 21 |
| 16 | BAIAP3 | BAI1 associated protein 3 | 007516 | 103275 | 007520 | 8, 31, 42 |
| 16 | BBS2 | Bardet-Biedl syndrome 2 | 125124 | 087263 | 102891 | 31, 2, 26 |
| 16 | BCAR1 | BCAR1, Cas family scaffolding protein | 050820 | 168925 | 153774 | 3, 6, 26 |
| 16 | BCL7C | BCL tumor suppressor 7C | 099385 | 102870 | 150281 | 5, |
| 16 | BCO1 | beta-carotene oxygenase 1 | 135697 | 279362 | 261609 | 14, 17, 16 |
| 16 | BICDL2 | BICD family like cargo adapter 2 | 162069 | 131652 | 008516 | 27, 13, 37 |
| 16 | BFAR | bifunctional apoptosis regulator | 103429 | 140694 | 069764 | 35, 40, 11 |
| 16 | BOLA2 | bolA family member 2 | 183336 | 254206 | 261740 | |
| 16 | BOLA2B | bolA family member 2B | 169627 | 102879 | 132207 | |
| 16 | BEAN1 | brain expressed, associated with NEDD4, 1 | 166546 | 179776 | 260851 | |
| 16 | BCKDK | branched chain ketoacid dehydrogenase kinase | 103507 | 167397 | 103510 | 34, 40, 2 |
| 16 | BRICD5 | BRICHOS domain containing 5 | 182685 | 167965 | 184207 | 2, 3, 23 |
| 16 | BRD7 | bromodomain containing 7 | 166164 | 121281 | 140807 | 15, 40, 6 |
| 16 | BANP | BTG3 associated nuclear protein | 172530 | 174990 | 225614 | |
| 16 | C1QTNF8 | C1q and tumor necrosis factor related protein 8 | 184471 | 162009 | 196557 | 10, 16, |
| 16 | CDH1 | cadherin 1 | 039068 | 062038 | 103047 | 23, 18, 37 |
| 16 | CDH11 | cadherin 11 | 140937 | 150394 | 179776 | 18, 11, 42 |
| 16 | CDH13 | cadherin 13 | 140945 | 135698 | 230989 | 10, 42, 44 |
| 16 | CDH15 | cadherin 15 | 129910 | 176715 | 259803 | 25, 15, 32 |
| 16 | CDH16 | cadherin 16 | 166589 | 172840 | 166592 | 17, 40, 25 |
| 16 | CDH3 | cadherin 3 | 062038 | 184939 | 039068 | 42, |
| 16 | CDH5 | cadherin 5 | 179776 | 140937 | 166546 | 32, 45, 40 |
| 16 | CDH8 | cadherin 8 | 150394 | 278499 | 140937 | 26, 10, 12 |
| 16 | CALB2 | calbindin 2 | 172137 | 180917 | 167377 | 1, 26, 31 |
| 16 | CHP2 | calcineurin like EF-hand protein 2 | 166869 | 134398 | 166501 | 16, 24, 17 |
| 16 | CPPED1 | calcineurin like phosphoesterase domain containing 1 | 103381 | 048471 | 237515 | 5, 26, 45 |
| 16 | CARHSP1 | calcium regulated heat stable protein 1 | 153048 | 140650 | 283516 | 38, 29, 1 |
| 16 | CACNG3 | calcium voltage-gated channel auxiliary subunit gamma 3 | 006116 | 166501 | 122257 | 8, 14, 17 |
| 16 | CACNA1H | calcium voltage-gated channel subunit alpha1 H | 196557 | 184471 | 116176 | 28, 29, 33 |
| 16 | CAPN15 | calpain 15 | 103326 | 090565 | 161992 | 34, 3, 35 |
| 16 | CAPNS2 | calpain small subunit 2 | 256812 | 087253 | 103546 | 4, 43, 18 |
| 16 | CMTR2 | cap methyltransferase 2 | 180917 | 157423 | 172137 | |
| 16 | CARMIL2 | capping protein regulator and myosin 1 linker 2 | 159753 | 102974 | 102977 | 22, 41, 39 |
| 16 | CHST4 | carbohydrate sulfotransferase 4 | 140835 | 157429 | 198650 | 4, 42, 25 |
| 16 | CHST5 | carbohydrate sulfotransferase 5 | 135702 | 260092 | 205084 | 12, 38, |
| 16 | CHST6 | carbohydrate sulfotransferase 6 | 183196 | 166822 | 260092 | 31, 42, 18 |
| 16 | CA5A | carbonic anhydrase 5A | 174990 | 103257 | 172530 | 19, |
| 16 | CA7 | carbonic anhydrase 7 | 168748 | 159593 | 172840 | 8, 16, |
| 16 | CES1 | carboxylesterase 1 | 198848 | 103546 | 159398 | 18, 13, 19 |
| 16 | CES2 | carboxylesterase 2 | 172831 | 166595 | 172828 | 21, |
| 16 | CES3 | carboxylesterase 3 | 172828 | 172831 | 172824 | 42, 16, 23 |
| 16 | CES4A | carboxylesterase 4A | 172824 | 172828 | 067955 | 11, 26, 29 |
| 16 | CES5A | carboxylesterase 5A | 159398 | 198848 | 087258 | 38, 39, 8 |
| 16 | CTF1 | cardiotrophin 1 | 150281 | 099385 | 099364 | 26, 14, 3 |
| 16 | CSNK2A2 | casein kinase 2 alpha 2 | 070770 | 070761 | 103021 | 6, 15, 4 |
| 16 | CASKIN1 | CASK interacting protein 1 | 167971 | 131653 | 167965 | 26, 33, 14 |
| 16 | CBFA2T3 | CBFA2/RUNX1 translocation partner 3 | 129993 | 205022 | 176715 | 21, 41, 39 |
| 16 | CCL17 | C-C motif chemokine ligand 17 | 102970 | 006210 | 005194 | 23, |
| 16 | CCL22 | C-C motif chemokine ligand 22 | 102962 | 102934 | 006210 | 30, 5, 39 |
| 16 | CTCF | CCCTC-binding factor | 102974 | 283332 | 159753 | 39, 21, 13 |
| 16 | CNOT1 | CCR4-NOT transcription complex subunit 1 | 125107 | 103037 | 279816 | |
| 16 | CD19 | CD19 molecule | 177455 | 177548 | 176953 | 41, 36, 22 |
| 16 | CD2BP2 | CD2 cytoplasmic tail binding protein 2 | 169217 | 198064 | 169221 | 21, 34, 20 |
| 16 | CDIPT | CDP-diacylglycerol-inositol 3-phosphatidyltransferase | 103502 | 013364 | 174938 | |
| 16 | CDIP1 | cell death inducing p53 target 1 | 089486 | 103415 | 205832 | 8, 26, 14 |
| 16 | CEMP1 | cementum protein 1 | 205923 | 162066 | 140992 | 29, 37, 14 |
| 16 | CENPBD1 | CENPB DNA-binding domain containing 1 | 177946 | 140995 | 003249 | |
| 16 | CCP110 | centriolar coiled-coil protein 110 | 103540 | 006007 | 103544 | 11, 31, 8 |
| 16 | CENPN | centromere protein N | 166451 | 103121 | 166454 | 3, 33, 22 |
| 16 | CENPT | centromere protein T | 102901 | 102904 | 168286 | 39, 36, 13 |
| 16 | CDR2 | cerebellar degeneration related protein 2 | 140743 | 058600 | 243716 | |
| 16 | CBLN1 | cerebellin 1 precursor | 102924 | 102921 | 166152 | 38, 12, 8 |
| 16 | CHMP1A | charged multivesicular body protein 1A | 131165 | 015413 | 167523 | 9, 21, 38 |
| 16 | CKLF | chemokine like factor | 217555 | 166548 | 254788 | 11, 29, 5 |
| 16 | CLCN7 | chloride voltage-gated channel 7 | 103249 | 197599 | 251692 | 7, 22, 39 |
| 16 | CETP | cholesteryl ester transfer protein | 087237 | 051108 | 140853 | 29, 36, 30 |
| 16 | CDT1 | chromatin licensing and DNA replication factor 1 | 167513 | 103335 | 198931 | 9, 21, 5 |
| 16 | CHD9 | chromodomain helicase DNA binding protein 9 | 177200 | 277639 | 279018 | |
| 16 | CDYL2 | chromodomain Y-like 2 | 166446 | 168589 | 103121 | 32, 23, 33 |
| 16 | C16orf45 | chromosome 16 open reading frame 45 | 166780 | 261130 | 166783 | |
| 16 | C16orf46 | chromosome 16 open reading frame 46 | 166455 | 166454 | 140905 | 11, 10, 42 |
| 16 | C16orf52 | chromosome 16 open reading frame 52 | 185716 | 155714 | 175267 | |
| 16 | C16orf54 | chromosome 16 open reading frame 54 | 185905 | 103485 | 174992 | 36, 5, 30 |
| 16 | C16orf58 | chromosome 16 open reading frame 58 | 140688 | 140675 | 169877 | |
| 16 | C16orf59 | chromosome 16 open reading frame 59 | 162062 | 162063 | 162068 | 39, 5, 26 |
| 16 | C16orf62 | chromosome 16 open reading frame 62 | 103544 | 103540 | 103550 | 24, 40, 39 |
| 16 | C16orf70 | chromosome 16 open reading frame 70 | 125149 | 067955 | 237172 | 26, 31, 16 |
| 16 | C16orf71 | chromosome 16 open reading frame 71 | 166246 | 168096 | 103199 | 38, 42, 27 |
| 16 | C16orf72 | chromosome 16 open reading frame 72 | 182831 | 187555 | 183454 | 34, 29, 22 |
| 16 | C16orf74 | chromosome 16 open reading frame 74 | 154102 | 131153 | 131148 | 34, 29, 32 |
| 16 | C16orf78 | chromosome 16 open reading frame 78 | 166152 | 102924 | 102935 | 38, 8, |
| 16 | C16orf82 | Chromosome 16 open reading frame 82 | 234186 | 182601 | 155666 | 38, 8, |
| 16 | C16orf86 | chromosome 16 open reading frame 86 | 159761 | 124074 | 141098 | 29, 26, 38 |
| 16 | C16orf87 | chromosome 16 open reading frame 87 | 155330 | 140795 | 166123 | 38, 7, 1 |
| 16 | C16orf89 | chromosome 16 open reading frame 89 | 153446 | 033011 | 118894 | 26, 40, 13 |
| 16 | C16orf90 | chromosome 16 open reading frame 90 | 215131 | 122390 | 103351 | 38, 33, 12 |
| 16 | C16orf91 | chromosome 16 open reading frame 91 | 174109 | 059145 | 197599 | 6, 3, 22 |
| 16 | C16orf92 | chromosome 16 open reading frame 92 | 167194 | 149927 | 149926 | 14, 38, |
| 16 | C16orf95 | chromosome 16 open reading frame 95 | 260456 | 176678 | 131152 | 38, |
| 16 | C16orf96 | chromosome 16 open reading frame 96 | 205832 | 089486 | 153443 | 38, 35, |
| 16 | CHTF18 | chromosome transmission fidelity factor 18 | 127586 | 007376 | 127588 | 27, 6, 12 |
| 16 | CHTF8 | chromosome transmission fidelity factor 8 | 168802 | 103044 | 141076 | 13, 3, 21 |
| 16 | CTRL | chymotrypsin like | 141086 | 159792 | 261884 | 4, 28, 22 |
| 16 | CTRB1 | chymotrypsinogen B1 | 168925 | 168928 | 050820 | 28, 4, 36 |
| 16 | CTRB2 | chymotrypsinogen B2 | 168928 | 184517 | 168925 | 28, 4, 36 |
| 16 | CFAP20 | cilia and flagella associated protein 20 | 070761 | 102996 | 070770 | 31, 37, 13 |
| 16 | CMTM1 | CKLF like MARVEL transmembrane domain containing 1 | 089505 | 254788 | 140932 | |
| 16 | CMTM2 | CKLF like MARVEL transmembrane domain containing 2 | 140932 | 089505 | 140931 | 38, 1, 8 |
| 16 | CMTM3 | CKLF like MARVEL transmembrane domain containing 3 | 140931 | 140932 | 183723 | 32, 5, 38 |
| 16 | CMTM4 | CKLF like MARVEL transmembrane domain containing 4 | 183723 | 140931 | 135720 | 29, 40, 16 |
| 16 | CKLF-CMTM1 | CKLF-CMTM1 readthrough | 254788 | 217555 | 089505 | |
| 16 | CIITA | class II major histocompatibility complex transactivator | 179583 | 166676 | 182108 | 22, 21, 1 |
| 16 | CLDN6 | claudin 6 | 184697 | 213937 | 006327 | 12, |
| 16 | CLDN9 | claudin 9 | 213937 | 270168 | 184697 | 31, 20, 16 |
| 16 | CLN3 | CLN3, battenin | 188603 | 233232 | 283220 | |
| 16 | CLN3 | CLN3, battenin | 283220 | 188603 | 184730 | 9, 29, 32 |
| 16 | CLUAP1 | clusterin associated protein 1 | 103351 | 215131 | 167984 | 30, 29, 14 |
| 16 | CMIP | c-Maf inducing protein | 153815 | 261609 | 197943 | 26, 20, 14 |
| 16 | COTL1 | coactosin like F-actin binding protein 1 | 103187 | 140950 | 135686 | 41, 3, 40 |
| 16 | COQ7 | coenzyme Q7, hydroxylase | 167186 | 170537 | 205730 | |
| 16 | COQ9 | coenzyme Q9 | 088682 | 005194 | 102978 | 9, 25, 21 |
| 16 | CCDC102A | coiled-coil domain containing 102A | 135736 | 125170 | 159618 | 1, 18, 9 |
| 16 | CCDC113 | coiled-coil domain containing 113 | 103021 | 070770 | 103023 | 40, 11, 9 |
| 16 | CCDC154 | coiled-coil domain containing 154 | 197599 | 174109 | 103249 | 25, 39, 21 |
| 16 | CCDC189 | coiled-coil domain containing 189 | 196118 | 156873 | 103549 | 39, 37, 22 |
| 16 | CCDC78 | coiled-coil domain containing 78 | 162004 | 103254 | 103253 | 42, 37, 20 |
| 16 | COG4 | component of oligomeric golgi complex 4 | 103051 | 157353 | 189091 | 1, 43, 3 |
| 16 | COG7 | component of oligomeric golgi complex 7 | 168434 | 168447 | 103365 | 9, 34, 4 |
| 16 | COG8 | component of oligomeric golgi complex 8 | 213380 | 132612 | 272617 | 38, 41, 9 |
| 16 | COG8 | component of oligomeric golgi complex 8 | 272617 | 213380 | 258429 | 38, 41, 9 |
| 16 | CNTNAP4 | contactin associated protein like 4 | 152910 | 166848 | 261833 | 8, 38, 25 |
| 16 | CPNE2 | copine 2 | 140848 | 140853 | 172775 | 11, 5, 43 |
| 16 | CPNE7 | copine 7 | 178773 | 167526 | 015413 | 3, 16, 37 |
| 16 | CBFB | core-binding factor beta subunit | 067955 | 172824 | 125149 | 34, 43, 29 |
| 16 | CORO7-PAM16 | CORO7-PAM16 readthrough | 103426 | 217930 | 262246 | |
| 16 | CORO1A | coronin 1A | 102879 | 102882 | 169627 | 41, 39, 21 |
| 16 | CORO7 | coronin 7 | 262246 | 103426 | 168140 | |
| 16 | CRAMP1 | cramped chromatin regulator homolog 1 | 007545 | 131634 | 261732 | 40, 22, 18 |
| 16 | CFDP1 | craniofacial development protein 1 | 153774 | 050820 | 261717 | 31, 30, 34 |
| 16 | CREBBP | CREB binding protein | 005339 | 126602 | 162104 | 18, 23, 22 |
| 16 | CRYM | crystallin mu | 103316 | 175311 | 169246 | 11, 31, 8 |
| 16 | CNEP1R1 | CTD nuclear envelope phosphatase 1 regulatory subunit 1 | 205423 | 102935 | 155393 | 39, 38, |
| 16 | CLEC16A | C-type lectin domain family 16 member A | 038532 | 182108 | 175643 | 38, 39, 21 |
| 16 | CLEC18A | C-type lectin domain family 18 member A | 157322 | 198373 | 279533 | 17, 40, 38 |
| 16 | CLEC18B | C-type lectin domain family 18 member B | 140839 | 196436 | 090863 | |
| 16 | CLEC18C | C-type lectin domain family 18 member C | 157335 | 090857 | 280006 | 17, 38, |
| 16 | CLEC19A | C-type lectin domain family 19 member A | 261210 | 103528 | 103534 | 12, |
| 16 | CLEC3A | C-type lectin domain family 3 member A | 166509 | 171724 | 186153 | |
| 16 | CX3CL1 | C-X3-C motif chemokine ligand 1 | 006210 | 102962 | 102970 | 8, 20, 1 |
| 16 | CMC2 | C-X9-C motif containing 2 | 103121 | 166446 | 166451 | 43, 29, 22 |
| 16 | CNGB1 | cyclic nucleotide gated channel beta 1 | 070729 | 140859 | 159648 | 14, 9, 8 |
| 16 | CDK10 | cyclin dependent kinase 10 | 185324 | 167523 | 158792 | 9, 40, 32 |
| 16 | CCNF | cyclin F | 162063 | 167972 | 162062 | 21, 39, 4 |
| 16 | CYLD | CYLD lysine 63 deubiquitinase | 083799 | 167207 | 103449 | |
| 16 | CRISPLD2 | cysteine rich secretary protein LCCL domain containing 2 | 103196 | 103194 | 153786 | 42, 44, 32 |
| 16 | CYBA | cytochrome b-245 alpha chain | 051523 | 124391 | 167508 | 41, 22, 5 |
| 16 | CYB5B | cytcchrome b5 type B | 103018 | 132604 | 102908 | 2, 9, 21 |
| 16 | COX4I1 | cytechrome c cxidase subunit 4I1 | 131143 | 131148 | 140968 | 43, 29, 41 |
| 16 | COX6A2 | cytcchrome c oxidase subunit 6A2 | 156885 | 156886 | 176723 | 25, 15, 33 |
| 16 | CIAPIN1 | cytokine induced apoptosis inhibitor 1 | 005194 | 102970 | 088682 | 21, 30, 9 |
| 16 | CTU2 | cytosolic thiouridylase subunit 2 | 174177 | 158717 | 103335 | 21, 22, 31 |
| 16 | DCTPP1 | dCTP pyrophosphatase 1 | 179958 | 179965 | 179918 | 1, 3, 9 |
| 16 | DDX19A | DEAD-box helicase 19A | 168872 | 260537 | 157350 | 24, 35, 21 |
| 16 | DDX19B | DEAD-box helicase 19B | 157349 | 090861 | 260537 | 13, 33, 38 |
| 16 | DDX28 | DEAD-box helicase 28 | 182810 | 263201 | 072736 | 7, 3, 37 |
| 16 | DHX38 | DEAH-box helicase 38 | 140829 | 261701 | 118557 | 42, 21, 1 |
| 16 | DCUN1D3 | defective in cullin neddylation 1 domain containing 3 | 188215 | 005189 | 102897 | 1, 3, 5 |
| 16 | DNASE1 | deoxyribonudease 1 | 213918 | 188827 | 126602 | |
| 16 | DNASE1L2 | deoxyribonudease 1 like 2 | 167968 | 167967 | 167969 | 35, 14, 6 |
| 16 | DEXI | Dexi homolog | 182108 | 179583 | 038532 | 36, 34, 29 |
| 16 | DEF8 | differentially expressed in FDCP 8 homolog | 140995 | 258947 | 177946 | 8, 42, 41 |
| 16 | DHODH | dihydroorotate dehydrogenase (quinone) | 102967 | 277481 | 140830 | 3, 31, 40 |
| 16 | DUS2 | dihydrouridine synthase 2 | 167264 | 167261 | 263201 | 21, 3, 9 |
| 16 | DPEP1 | dipeptidase 1 (renal) | 015413 | 178773 | 131165 | 16, 22, 17 |
| 16 | DPEP2 | dipeptidase 2 | 167261 | 141096 | 167264 | 5, 36, 32 |
| 16 | DPEP3 | dipeptidase 3 | 141096 | 124067 | 167261 | 38, 44, 17 |
| 16 | DNAJA2 | DnaJ heat shock protein family (Hsp40) member A2 | 069345 | 166123 | 171208 | 18, 6, 40 |
| 16 | DNAJA3 | DnaJ heat shock protein family (Hsp40) member A3 | 103423 | 168140 | 153406 | 13, 34, 3 |
| 16 | DOK4 | docking protein 4 | 125170 | 102978 | 135736 | 8, 45, 16 |
| 16 | DOC2A | double C2 domain alpha | 149927 | 169592 | 167194 | 38, |
| 16 | DCTN5 | dynactin subunit 5 | 166847 | 083093 | 166851 | 42, 24, 39 |
| 16 | DNAAF1 | dynein axonemal assembly factor 1 | 154099 | 103160 | 103168 | 38, 42, 32 |
| 16 | DNAH3 | dynein axonemal heavy chain 3 | 158486 | 102897 | 011638 | 42, 38, 27 |
| 16 | DYNC1LI2 | dynein cytoplasmic 1 light intermediate chain 2 | 135720 | 183723 | 249961 | 29, 42, 13 |
| 16 | DYNLRB2 | dynein light chain roadblock-type 2 | 168589 | 178573 | 166446 | 38, 30, 17 |
| 16 | DRC7 | dynein regulatory complex subunit 7 | 159625 | 182885 | 140854 | 38, 42, 17 |
| 16 | DBNDD1 | dysbindin domain containing 1 | 003249 | 177946 | 141013 | 33, 4, 8 |
| 16 | E2F4 | E2F transcription factor 4 | 205250 | 179044 | 102890 | 3, 9, 29 |
| 16 | E4F1 | E4F transcription factor 1 | 167967 | 184207 | 167968 | 1, 41, 38 |
| 16 | ERN2 | endoplasmic reticulum to nucleus signaling 2 | 134398 | 166851 | 166869 | 42, 3, 16 |
| 16 | ELMO3 | engulfment and cell motility 3 | 102890 | 205250 | 125122 | 3, 13, 40 |
| 16 | EDC4 | enhancer of mRNA decapping 4 | 038358 | 102898 | 188038 | 26, 18, 39 |
| 16 | ENKD1 | enkurin domain containing 1 | 124074 | 102981 | 159761 | 9, 6, 5 |
| 16 | ECI1 | enoyl-CoA delta isomerase 1 | 167969 | 167968 | 205937 | 28, 14, 20 |
| 16 | EMP2 | epithelial membrane protein 2 | 213853 | 166669 | 153060 | 29, 18, 20 |
| 16 | ESRP2 | epithelial splicing regulatory protein 2 | 103067 | 072736 | 103066 | 43, 18, 38 |
| 16 | EMC8 | ER membrane protein complex subunit 8 | 131148 | 154102 | 131143 | 34, 43, 33 |
| 16 | ERCC4 | ERCC excision repair 4, endonuclease catalytic subunit | 175595 | 237515 | 186260 | 23, 38, 6 |
| 16 | ERI2 | ERI1 exoribonuclease family member 2 | 196678 | 066654 | 005189 | 33, 11, 9 |
| 16 | EME2 | essential meiotic structure-specific endonuclease subunit 2 | 197774 | 074071 | 162032 | 26, 12, 37 |
| 16 | EEF2K | eukaryotic elongation factor 2 kinase | 103319 | 183921 | 058600 | 1, 34, 35 |
| 16 | EEF2KMT | eukaryotic elongation factor 2 lysine methyltransferase | 118894 | 153446 | 078328 | 11, 34, 38 |
| 16 | EIF3C | eukaryotic translation initiation factor 3 subunit C | 184110 | 255524 | 196993 | 39, 43, 21 |
| 16 | EIF3CL | eukaryotic translation initiation factor 3 subunit C-like | 205609 | 198156 | 261832 | 43, 1, 3 |
| 16 | EXOC3L1 | exocyst complex component 3 like 1 | 179044 | 196123 | 205250 | 36, 18, 27 |
| 16 | EXOSC6 | exosome component 6 | 223496 | 280252 | 090861 | |
| 16 | XPO6 | exportin 6 | 169180 | 169181 | 188322 | 43, 9, 21 |
| 16 | FAM173A | family with sequence similarity 173 member A | 103254 | 103260 | 162004 | 34, 6, 20 |
| 16 | FAM192A | family with sequence similarity 192 member A | 172775 | 140848 | 159579 | 9, 3, 6 |
| 16 | FAM234A | family with sequence similarity 234 member A | 167930 | 007392 | 269881 | 37, 9, 33 |
| 16 | FAM57B | family with sequence similarity 57 member B | 149926 | 167194 | 149925 | 38, 22, 8 |
| 16 | FAM65A | family with sequence similarity 65 member A | 039523 | 159723 | 283332 | 43, 40, 26 |
| 16 | FAM92B | family with sequence similarity 92 member B | 153789 | 135709 | 131149 | 42, 10, 20 |
| 16 | FAM96B | family with sequence similarity 96 member B | 166595 | 166592 | 172831 | 28, 34, 35 |
| 16 | FANCA | Fanconi anemia complementation group A | 187741 | 158805 | 204991 | 9, 21, 43 |
| 16 | FA2H | fatty acid 2-hydroxylase | 103089 | 168404 | 103091 | 37, 9, 3 |
| 16 | FBXL16 | F-box and leucine rich repeat protein 16 | 127585 | 127580 | 103260 | 28, 8, 26 |
| 16 | FBXL19 | F-box and leucine rich repeat protein 19 | 099364 | 150281 | 175938 | 12, 2, 28 |
| 16 | FBXL8 | F-box and leucine rich repeat protein 8 | 135722 | 102871 | 265690 | 34, 42, 9 |
| 16 | FBXO31 | F-box protein 31 | 103264 | 131152 | 283277 | 33, 22, 15 |
| 16 | FBXO31 | F-box protein 31 | 283277 | 103264 | 140941 | 33, 22, 15 |
| 16 | FOPNL | FGFR1OP N-terminal like | 133393 | 133392 | 103222 | 45, 13, 22 |
| 16 | FBRS | fibrosin | 156860 | 156858 | 080603 | 34, 41, 28 |
| 16 | FLYWCH2 | FLYWCH family member 2 | 162076 | 005001 | 059122 | 1, 18, 27 |
| 16 | FLYWCH1 | FLYWCH-type zinc finger 1 | 059122 | 162076 | 131650 | 31, 1, 14 |
| 16 | FOXC2 | forkhead box C2 | 176692 | 103248 | 176678 | 11, 45, 12 |
| 16 | FOXF1 | forkhead box F1 | 103241 | 140968 | 103248 | 9, 10, 37 |
| 16 | FOXL1 | forkhead box L1 | 176678 | 176692 | 260456 | 14, |
| 16 | FHOD1 | formin homology 2 domain containing 1 | 135723 | 168701 | 135740 | 34, 7, 40 |
| 16 | FTO | FTO, alpha-ketoglutarate dependent dioxygenase | 140718 | 103494 | 177508 | 9, 18, 8 |
| 16 | FUK | fucokinase | 157353 | 157350 | 103051 | 42, 39, 18 |
| 16 | FAHD1 | fumarylacetoacetate hydrolase domain containing 1 | 180185 | 063854 | 162039 | 23, 2, 11 |
| 16 | FUS | FUS RNA binding protein | 089280 | 178226 | 103490 | 21, 12, 40 |
| 16 | GNAO1 | G protein subunit alpha o1 | 087258 | 159398 | 159461 | 8, 14, 20 |
| 16 | GNG13 | G protein subunit gamma 13 | 127588 | 127586 | 167945 | 14, 33, 8 |
| 16 | GPR139 | G protein-coupled receptor 139 | 180269 | 167191 | 169347 | |
| 16 | GPRC5B | G protein-coupled receptor class C group 5 member B | 167191 | 174628 | 180269 | 14, |
| 16 | GSPT1 | G1 to S phase transition 1 | 103342 | 171490 | 234719 | 9, 40, 33 |
| 16 | GABARAPL2 | GABA type A receptor associated protein like 2 | 034713 | 205084 | 065457 | 31, 5, 8 |
| 16 | GALNS | galactosamine (N-acetyl)-6-sulfatase | 141012 | 198931 | 280675 | 9, 1, 3 |
| 16 | GTF3C1 | general transcription factor IIIC subunit 1 | 077235 | 103522 | 047578 | |
| 16 | GAN | gigaxonin | 261609 | 135697 | 153815 | 13, 23, 24 |
| 16 | GINS2 | GINS complex subunit 2 | 131153 | 131149 | 154102 | 21, 22, 9 |
| 16 | GINS3 | GINS complex subunit 3 | 181938 | 103023 | 103034 | 43, 21, 26 |
| 16 | GLIS2 | GLIS family zinc finger 2 | 126603 | 090447 | 217930 | 1, 28, 34 |
| 16 | GFOD2 | glucose-fructose oxidoreductase domain containing 2 | 141098 | 159761 | 141084 | 41, 13, 9 |
| 16 | GRIN2A | glutamate ionotropic receptor NMDA type subunit 2A | 183454 | 182831 | 166669 | 3, 17, 25 |
| 16 | GOT2 | glutamic-oxaloacetic transaminase 2 | 125166 | 103042 | 278499 | 15, 24, 21 |
| 16 | GPT2 | glutamic-pyruvic transaminase 2 | 166123 | 155330 | 069345 | 1, 11, 33 |
| 16 | EARS2 | glutamyl-tRNA synthetase 2, mitochondrial | 103356 | 103365 | 103353 | 39, 9, 24 |
| 16 | GDE1 | glycerophosphodiester phosphodiesterase 1 | 006007 | 103534 | 103540 | 43, 33, 38 |
| 16 | GDPD3 | glycerophosphodiester phosphodiesterase domain containing 3 | 102886 | 090238 | 102882 | 30, 24, 15 |
| 16 | GCSH | glycine cleavage system protein H | 140905 | 166455 | 166473 | 17, 43, 34 |
| 16 | GP2 | glycoprotein 2 | 169347 | 180269 | 169344 | 4, 28, 19 |
| 16 | GLYR1 | glyoxylate reductase 1 homolog | 140632 | 067836 | 118900 | 22, 15, 38 |
| 16 | GGA2 | golgi associated, gamma adaptin ear containing, ARF binding protein 2 | 103365 | 168434 | 103356 | |
| 16 | GLG1 | golgi glycoprotein 1 | 090863 | 140839 | 168411 | 42, 40, 38 |
| 16 | GAS8 | growth arrest specific 8 | 141013 | 003249 | 283182 | |
| 16 | GFER | growth factor, augmenter of liver regeneration | 127554 | 196408 | 127561 | 34, 6, 14 |
| 16 | GSE1 | Gse1 coiled-coil protein | 131149 | 153789 | 131153 | |
| 16 | GSG1L | GSG1 like | 169181 | 047578 | 169180 | 14, 8, 38 |
| 16 | HP | haptoglobin | 257017 | 140830 | 261701 | 19, 5, 1 |
| 16 | HPR | haptoglobin-related protein | 261701 | 257017 | 140829 | 19, 15, 8 |
| 16 | HEATR3 | HEAT repeat containing 3 | 155393 | 205423 | 121274 | 23, 31, 45 |
| 16 | HSBP1 | heat shock factor binding protein 1 | 230989 | 140945 | 103150 | 29, 15, 5 |
| 16 | HSF4 | heat shock transcription factor 4 | 102878 | 265690 | 140939 | 29, 40, 15 |
| 16 | HN1L | hematological and neurological expressed 1 like | 206053 | 261732 | 138834 | 24, 42, 34 |
| 16 | HMOX2 | heme oxygenase 2 | 103415 | 153406 | 089486 | 21, 29, 1 |
| 16 | HBA1 | hemoglobin subunit alpha 1 | 206172 | 188536 | 086506 | 36, 30, 19 |
| 16 | HBA2 | hemoglobin subunit alpha 2 | 188536 | 206177 | 206172 | 36, 32, 7 |
| 16 | HBM | hemoglobin subunit mu | 206177 | 130656 | 188536 | 19, 22, 10 |
| 16 | HBQ1 | hemoglobin subunit theta 1 | 086506 | 206172 | 007392 | 5, 33, 38 |
| 16 | HBZ | hemoglobin subunit zeta | 130656 | 103148 | 206177 | 36, 39, 15 |
| 16 | HS3ST2 | heparan sulfate-glucosamine 3-sulfotransferase 2 | 122254 | 243716 | 103404 | |
| 16 | HS3ST4 | heparan sulfate-glucosamine 3-sulfotransferase 4 | 182601 | 155592 | 234186 | 20, 14, 6 |
| 16 | HS3ST6 | heparan sulfate-glucosamine 3-sulfotransferase 6 | 162040 | 162039 | 198736 | 15, 14, 33 |
| 16 | HIRIP3 | HIRA interacting protein 3 | 149929 | 149930 | 169592 | 41, 3, 34 |
| 16 | HERPUD1 | homocysteine inducible ER protein with ubiquitin like domain 1 | 051108 | 070915 | 087237 | 41, 29, 37 |
| 16 | HCFC1R1 | host cell factor C1 regulator 1 | 103145 | 006327 | 131652 | 41, 25, 1 |
| 16 | HAS3 | hyaluronan synthase 3 | 103044 | 103047 | 168802 | 24, 40, 4 |
| 16 | HYDIN | HYDIN, axonemal central pair apparatus protein | 157423 | 103043 | 180917 | |
| 16 | HAGH | hydroxyacylglutathione hydrolase | 063854 | 099769 | 180185 | 31, 17, 38 |
| 16 | HAGHL | hydroxyacylglutathione hydrolase-like | 103253 | 162004 | 103245 | |
| 16 | HSD3B7 | hydroxy-delta-5-steroid dehydrogenase, 3 beta- and steroid delta-isomerase 7 | 099377 | 099381 | 099365 | 1, 9, 31 |
| 16 | HSD11B2 | hydroxysteroid 11-beta dehydrogenase 2 | 176387 | 159714 | 159720 | 32, 3, 16 |
| 16 | HSD17B2 | hydroxysteroid 17-beta dehydrogenase 2 | 086696 | 184860 | 135698 | 32, 7, 16 |
| 16 | HSDL1 | hydroxysteroid dehydrogenase like 1 | 103160 | 140943 | 154099 | 8, 42, 38 |
| 16 | IGHV2OR16-5 | Immunoglobulin heavy variable 2/OR16-5 (non-functional) | 259303 | 183632 | 233732 | |
| 16 | IGHV3OR16-10 | Immunoglobulin heavy variable 3/OR16-10 (non-functional) | 233732 | 259303 | 271130 | |
| 16 | IGHV3OR16-12 | Immunoglobulin heavy variable 3/OR16-12 (non-functional) | 270467 | 278848 | 271178 | |
| 16 | IGHV3OR16-13 | Immunoglobulin heavy variable 3/OR16-13 (non-functional) | 271178 | 270467 | 259680 | |
| 16 | IGHV3OR16-8 | Immunoglobulin heavy variable 3/OR16-8 (non-functional) | 271130 | 233732 | 205457 | |
| 16 | IGHV3OR16-9 | Immunoglobulin heavy variable 3/OR16-9 (non-functional) | 270472 | 185947 | 205456 | |
| 16 | IGSF6 | immunoglobulin superfamily member 6 | 140749 | 197006 | 155719 | 30, 13, 5 |
| 16 | INO80E | INO80 complex subunit E | 169592 | 149929 | 149927 | 3, 26, 1 |
| 16 | ITPRIPL2 | inositol 1,4,5-trisphosphate receptor interacting protein like 2 | 205730 | 167186 | 261427 | 22, 1, 29 |
| 16 | IGFALS | insulin like growth factor binding protein acid labile subunit | 099769 | 095906 | 063854 | 19, 39, 23 |
| 16 | ITFG1 | integrin alpha FG-GAP repeat containing 1 | 129636 | 171208 | 102893 | 26, 40, 18 |
| 16 | ITGAD | integrin subunit alpha D | 156886 | 140678 | 156885 | 36, 25, 20 |
| 16 | ITGAL | integrin subunit alpha L | 005844 | 179918 | 169957 | 39, 36, 22 |
| 16 | ITGAM | integrin subunit alpha M | 169896 | 169900 | 140678 | 7, 5, 26 |
| 16 | ITGAX | integrin subunit alpha X | 140678 | 169896 | 156886 | 7, 5, 18 |
| 16 | IRF8 | interferon regulatory factor 8 | 140968 | 131143 | 103241 | 21, 22, 41 |
| 16 | IL17C | interleukin 17C | 124391 | 158545 | 051523 | 20, 38, |
| 16 | IL21R | interleukin 21 receptor | 103522 | 077238 | 077235 | 39, 41, 21 |
| 16 | IL27 | interleukin 27 | 197272 | 184730 | 176046 | 20, |
| 16 | IL32 | interleukin 32 | 008517 | 008516 | 130182 | 45, 3, 37 |
| 16 | IL34 | interleukin 34 | 157368 | 189091 | 132613 | 36, 1, 8 |
| 16 | IL4R | interleukin 4 receptor | 077238 | 169189 | 103522 | 18, 22, 25 |
| 16 | IFT140 | intraflagellar transport 140 | 187535 | 100726 | 131634 | 27, 38, 12 |
| 16 | IQCK | IQ motif containing K | 174628 | 103550 | 167191 | 38, |
| 16 | IRX3 | iroquois homeobox 3 | 177508 | 140718 | 176842 | 34, 9, 20 |
| 16 | IRX5 | iroquois homeobox 5 | 176842 | 177508 | 159387 | 20, 6, 23 |
| 16 | IRX6 | iroquois homeobox 6 | 159387 | 176842 | 087245 | 39, 14, 20 |
| 16 | IST1 | IST1, ESCRT-III associated factor | 182149 | 224470 | 102984 | 36, 42, 39 |
| 16 | JMJD8 | jumonji domain containing 8 | 161999 | 103266 | 127580 | 34, 27, 3 |
| 16 | JPH3 | junctophilin 3 | 154118 | 140948 | 104731 | 14, 8, 2 |
| 16 | KATNB1 | katanin regulatory subunit B1 | 140854 | 159625 | 140859 | 25, 35, 5 |
| 16 | KLHDC4 | kelch domain containing 4 | 104731 | 154118 | 103257 | |
| 16 | KLHL36 | kelch like family member 36 | 135686 | 103187 | 103194 | 13, 37, 22 |
| 16 | KIAA0430 | KIAA0430 | 166783 | 166780 | 072864 | 18, 41, 22 |
| 16 | KIAA0513 | KIAA0513 | 135709 | 153786 | 153789 | 34, 27, 33 |
| 16 | KIAA0556 | KIAA0556 | 047578 | 077235 | 169181 | 40, 30, 33 |
| 16 | KIAA0895L | KIAA0895 like | 196123 | 140939 | 179044 | 13, 8, 33 |
| 16 | KIF22 | kinesin family member 22 | 079616 | 174992 | 103495 | 39, 21, 9 |
| 16 | KIFC3 | kinesin family member C3 | 140859 | 140854 | 070729 | |
| 16 | KREMEN2 | kringle containing transmembrane protein 2 | 131650 | 059122 | 127564 | 37, 9, 39 |
| 16 | LDHD | lactate dehydrogenase D | 166816 | 186187 | 184517 | 3, 14, 16 |
| 16 | LCAT | lecithin-cholesterol acyltransferase | 213398 | 205220 | 124067 | |
| 16 | LCMT1 | leucine carboxyl methyltransferase 1 | 205629 | 140750 | 103375 | 1, 31, 43 |
| 16 | LRRC29 | leucine rich repeat containing 29 | 125122 | 102890 | 168701 | 26, 31, 36 |
| 16 | LRRC36 | leucine rich repeat containing 36 | 159708 | 168676 | 159713 | 38, 1, 32 |
| 16 | LAT | linker for activation of T-cells | 213658 | 169682 | 254206 | 39, 5, 15 |
| 16 | LMF1 | lipase maturation factor 1 | 103227 | 167945 | 005513 | 2, 42, 8 |
| 16 | LITAF | lipopolysaccharide induced TNF factor | 189067 | 188897 | 184602 | 5, 36, 11 |
| 16 | LONP2 | lon peptidase 2, peroxisomal | 102910 | 121270 | 196470 | 24, 40, 23 |
| 16 | LUC7L | LUC7 like | 007392 | 086506 | 167930 | 39, 36, 1 |
| 16 | LYRM1 | LYR metif containing 1 | 102897 | 188215 | 158486 | 29, 10, 2 |
| 16 | KAT8 | lysine acetyltransferase 8 | 103510 | 103507 | 052344 | 11, 18, 29 |
| 16 | KDM8 | lysine demethylase 8 | 155666 | 234186 | 169189 | 21, 2, 19 |
| 16 | KNOP1 | lysine rich nucleolar protein 1 | 103550 | 103544 | 174628 | 9, 11, 19 |
| 16 | LPCAT2 | lysophosphatidylcholine acyltransferase 2 | 087253 | 087245 | 256812 | 26, 29, 17 |
| 16 | KARS | lysyl-tRNA synthetase | 065427 | 065457 | 166848 | 3, 21, 9 |
| 16 | MAF | MAF bZIP transcription factor | 178573 | 186153 | 168589 | 11, 29, 17 |
| 16 | MGRN1 | mahogunin ring finger 1 | 102858 | 153443 | 168101 | 34, 26, 23 |
| 16 | MVP | major vault protein | 013364 | 281348 | 103502 | 24, 36, 39 |
| 16 | MLYCD | malonyl-CoA decarboxylase | 103150 | 230989 | 260300 | 2, 1, 26 |
| 16 | MCRIP2 | MAPK regulated corepressor interacting protein 2 | 172366 | 130731 | 161996 | |
| 16 | MARVELD3 | MARVEL domain containing 3 | 140832 | 198650 | 040199 | 37, 23, 29 |
| 16 | MMP15 | matrix metallopeptidase 15 | 102996 | 103005 | 070761 | 3, 26, 37 |
| 16 | MMP2 | matrix metallopeptidase 2 | 087245 | 159387 | 087253 | |
| 16 | MMP25 | matrix metallopeptidase 25 | 008516 | 162069 | 008517 | 36, 5, 39 |
| 16 | MEFV | MEFV, pyrin innate immunity regulator | 103313 | 010539 | 006194 | 5, 38, |
| 16 | MEIOB | meiosis specific with OB domains | 162039 | 180185 | 162040 | 38, 6, 10 |
| 16 | MC1R | melanocortin 1 receptor | 258839 | 141002 | 198211 | |
| 16 | MBTPS1 | membrane bound transcription factor peptidase, site 1 | 140943 | 166558 | 103160 | 42, 43, 31 |
| 16 | MSLN | mesothelin | 102854 | 103245 | 162006 | 27, 9, 1 |
| 16 | MSLNL | Mesothelin-like | 162006 | 102854 | 007376 | |
| 16 | MT1A | metallothionein 1A | 205362 | 205364 | 169688 | 16, 1, 14 |
| 16 | MT1B | metallothionein 1B | 169688 | 205362 | 198417 | 10, 5, |
| 16 | MT1E | metallothionein 1E | 169715 | 125148 | 205364 | 19, 31, 15 |
| 16 | MT1F | metallothionein 1F | 198417 | 169688 | 125144 | 40, 33, 18 |
| 16 | MT1G | metallothionein 1G | 125144 | 198417 | 205358 | 40, 19, 36 |
| 16 | MT1H | metallothionein 1H | 205358 | 125144 | 275691 | 19, 14, 40 |
| 16 | MT1M | metallothionein 1M | 205364 | 169715 | 205362 | 10, 44, 33 |
| 16 | MT1X | metallothionein 1X | 187193 | 275691 | 102900 | 25, 10, 19 |
| 16 | MT2A | metallothionein 2A | 125148 | 087250 | 169715 | 19, 1, 12 |
| 16 | MT3 | metallothionein 3 | 087250 | 102891 | 125148 | 8, 2, 10 |
| 16 | MT4 | metallothionein 4 | 102891 | 125124 | 087250 | 16, |
| 16 | METRN | meteorin, glial cell differentiation regulator | 103260 | 127585 | 103254 | |
| 16 | MTHFSD | methenyltetrahydrofolate synthetase domain containing | 103248 | 103241 | 176692 | 22, 13, 7 |
| 16 | MSRB1 | methionine sulfoxide reductase B1 | 198736 | 162040 | 140986 | 43, 17, 16 |
| 16 | METTL22 | methyltransferase like 22 | 067365 | 232258 | 183044 | 35, 6, 38 |
| 16 | METTL26 | methyltransferase like 26 | 130731 | 127578 | 172366 | |
| 16 | METTL9 | methyltransferase like 9 | 197006 | 169246 | 140749 | 29, 35, 6 |
| 16 | MVD | mevalonate diphosphate decarboxylase | 167508 | 051523 | 185669 | 21, 26, 41 |
| 16 | MAP1LC3B | microtubule associated protein 1 light chain 3 beta | 140941 | 283277 | 140948 | |
| 16 | MRPL28 | mitochondrial ribosomal protein L28 | 086504 | 103126 | 129925 | 35, 41, 25 |
| 16 | MRPS34 | mitochondrial ribosomal protein S34 | 074071 | 103024 | 197774 | 3, 14, 33 |
| 16 | MAPK3 | mitogen-activated protein kinase 3 | 102882 | 102886 | 102879 | 26, 21, 14 |
| 16 | MAPK8IP3 | mitogen-activated protein kinase 8 interacting protein 3 | 138834 | 206053 | 103024 | 26, 22, 16 |
| 16 | MLKL | mixed lineage kinase domain like pseudokinase | 168404 | 168411 | 103089 | 1, 18, 9 |
| 16 | MKL2 | MKL1/myocardin like 2 | 186260 | 175595 | 140694 | 11, 18, 34 |
| 16 | MON1B | MON1 homolog B, secretory trafficking associated | 103111 | 261833 | 205078 | 42, 39, 36 |
| 16 | MPHOSPH6 | M-phase phosphoprotein 6 | 135698 | 086696 | 140945 | 4, 11, 6 |
| 16 | MPV17L | MPV17 mitochondrial inner membrane protein like | 156968 | 183793 | 166780 | 4, 22, 33 |
| 16 | MLST8 | MTOR associated protein, LST8 homolog | 167965 | 167971 | 182685 | |
| 16 | MTRNR2L4 | MT-RNR2-like 4 | 232196 | 262621 | 140987 | |
| 16 | MTSS1L | MTSS1L, I-BAR domain containing | 132613 | 157368 | 103043 | 14, 18, 8 |
| 16 | MAZ | MYC associated zinc finger protein | 103495 | 079616 | 167371 | 23, 9, 41 |
| 16 | MYH11 | myosin heavy chain 11 | 133392 | 072864 | 133393 | 33, 16, 18 |
| 16 | MYLK3 | myosin light chain kinase 3 | 140795 | 091651 | 155330 | 15, 24, 25 |
| 16 | MYLPF | myosin light chain, phosphorylatable, fast skeletal muscle | 180209 | 169221 | 180035 | 25, 15, 24 |
| 16 | NAA60 | N(alpha)-acetyltransferase 60, NatF catalytic subunit | 122390 | 167981 | 215131 | 39, 24, 36 |
| 16 | GNPTG | N-acetylglucosamine-1-phosphate transferase gamma subunit | 090581 | 007520 | 059145 | 31, 35, 6 |
| 16 | NAGPA | N-acetylglucosamine-1-phosphodiester alpha-N-acetylglucosaminidase | 103174 | 103184 | 033011 | 9, 41, 36 |
| 16 | NQO1 | NAD(P)H quinone dehydrogenase 1 | 181019 | 102908 | 141101 | 3, 4, 24 |
| 16 | NDUFAB1 | NADH: ubiquinone oxidoreductase subunit AB1 | 004779 | 103353 | 083093 | 41, 13, 28 |
| 16 | NDUFB10 | NADH: ubiquinone oxidoreductase subunit B10 | 140990 | 140986 | 140988 | 25, 34, 31 |
| 16 | NOXO1 | NADPH oxidase organizer 1 | 196408 | 183751 | 127554 | 16, 4, 20 |
| 16 | NKD1 | naked cuticle homolog 1 | 140807 | 166164 | 167208 | 19, 21, 16 |
| 16 | NDRG4 | NDRG family member 4 | 103034 | 181938 | 103037 | 8, 26, 18 |
| 16 | N4BP1 | NEDD4 binding protein 1 | 102921 | 196470 | 102924 | 37, 43, 41 |
| 16 | NAE1 | NEDD8 activating enzyme E1 subunit 1 | 159593 | 249961 | 168748 | 3, 9, 6 |
| 16 | NTN3 | netrin 3 | 162068 | 162062 | 162065 | 16, |
| 16 | NRN1L | neuritin 1 like | 188038 | 038358 | 159792 | 14, 28, 12 |
| 16 | NPW | neuropeptide W | 183971 | 167962 | 065054 | 28, 22, 33 |
| 16 | NETO2 | neuropilin and tolloid like 2 | 171208 | 069345 | 129636 | 2, 40, 29 |
| 16 | NHLRC4 | NHL repeat containing 4 | 257108 | 007541 | 197562 | 2, 39, 36 |
| 16 | NOB1 | NIN1/PSMD8 binding protein 1 homolog | 141101 | 181019 | 198373 | 3, 21, 29 |
| 16 | NIP7 | NIP7, nucleolar pre-rRNA processing protein | 132603 | 259900 | 157315 | 7, 30, 4 |
| 16 | NLRC3 | NLR family CARD domain containing 3 | 167984 | 103351 | 188827 | 22, 1, 21 |
| 16 | NLRC5 | NLR family CARD domain containing 5 | 140853 | 087237 | 140848 | 43, 22, 36 |
| 16 | NME3 | NME/NM23 nucleoside diphosphate kinase 3 | 103024 | 138834 | 074071 | 3, 28, 29 |
| 16 | NME4 | NME/NM23 nucleoside diphosphate kinase 4 | 103202 | 129925 | 242612 | 33, 23, 1 |
| 16 | MPG | N-methylpurine DNA glycosylase | 103152 | 007384 | 103148 | 7, 43, 41 |
| 16 | NMRAL1 | NmrA like redox sensor 1 | 153406 | 103423 | 103415 | 38, |
| 16 | NOMO1 | NODAL modulator 1 | 103512 | 254852 | 183426 | 43, 9, 26 |
| 16 | NOMO2 | NODAL modulator 2 | 185164 | 233024 | 134419 | 43, 29, 31 |
| 16 | NOMO3 | NODAL modulator 3 | 103226 | 091262 | 183889 | 43, 29, 31 |
| 16 | NPRL3 | NPR3 like, GATOR1 complex subunit | 103148 | 103152 | 130656 | 7, 36, 14 |
| 16 | NSMCE1 | NSE1 homolog, SMC5-SMC6 complex component | 169189 | 155666 | 077238 | 43, 18, 33 |
| 16 | NTAN1 | N-terminal asparagine amidase | 157045 | 179889 | 085721 | 29, 34, 22 |
| 16 | NECAB2 | N-terminal EF-hand calcium binding protein 2 | 103154 | 140961 | 166558 | 8, 17, 10 |
| 16 | NTHL1 | nth like DNA glycosylase 1 | 065057 | 065054 | 103197 | 37, 16, 14 |
| 16 | NFATC2IP | nuclear factor of activated T-cells 2 interacting protein | 176953 | 177455 | 169682 | 14, 11, 36 |
| 16 | NFATC3 | nuclear factor of activated T-cells 3 | 072736 | 182810 | 103067 | 22, 41, 2 |
| 16 | NFAT5 | nuclear factor of activated T-cells 5 | 102908 | 103018 | 181019 | 18, 31, 22 |
| 16 | NPIPA1 | nuclear pore complex interacting protein family member A1 | 183426 | 103512 | 179889 | |
| 16 | NPIPA2 | nuclear pore complex interacting protein family member A2 | 254852 | 224712 | 103512 | 37, |
| 16 | NPIPA3 | nuclear pore complex interacting protein family member A3 | 224712 | 069764 | 254852 | 37, |
| 16 | NPIPA5 | nuclear pore complex interacting protein family member A5 | 183793 | 085721 | 261130 | |
| 16 | NPIPA7 | nuclear pore complex interacting protein family member A7 | 214967 | 183889 | 103489 | |
| 16 | NPIPA8 | nuclear pore complex interacting protein family member A8 | 214940 | 103489 | 233024 | |
| 16 | NPIPB11 | nuclear pore complex interacting protein family member B11 | 254206 | 213658 | 183336 | |
| 16 | NPIPB15 | nuclear pore complex interacting protein family member B15 | 196436 | 103035 | 140839 | 10, 14, 38 |
| 16 | NPIPB3 | nuclear pore complex interacting protein family member B3 | 169246 | 103316 | 197006 | |
| 16 | NPIPB4 | nuclear pore complex interacting protein family member B4 | 185864 | 155719 | 140740 | 26, 40, 30 |
| 16 | NPIPB5 | nuclear pore complex interacting protein family member B5 | 243716 | 140743 | 122254 | 8, |
| 16 | NPIPB6 | nuclear pore complex interacting protein family member B6 | 198156 | 188322 | 205609 | |
| 16 | NPIPB7 | Nuclear pore complex interacting protein family member B7 | 233232 | 261832 | 188603 | |
| 16 | NPIPB8 | nuclear pore complex interacting protein family member B8 | 255524 | 196502 | 184110 | |
| 16 | NPIPB9 | nuclear pore complex interacting protein family member B9 | 196993 | 184110 | 168488 | |
| 16 | NPIPB12 | Nuclear pore complex interacting protein family, member B12 | 169203 | 258150 | 197471 | |
| 16 | NPIPB13 | nuclear pore complex interacting protein family, member B13 | 198064 | 258130 | 169217 | 40, 36, 23 |
| 16 | NPIPB2 | Nuclear pore complex interacting protein family, member B2 | 234719 | 103342 | 048462 | 21, 9, |
| 16 | NARFL | nuclear prelamin A recognition factor like | 103245 | 103253 | 102854 | 9, 39, 36 |
| 16 | NUPR1 | nuclear protein 1, transcriptional regulator | 176046 | 197272 | 176476 | 29, 3, 6 |
| 16 | NUTF2 | nuclear transport factor 2 | 102898 | 168286 | 038358 | 41, 35, 44 |
| 16 | NOL3 | nucleolar protein 3 | 140939 | 102878 | 196123 | 34, 29, 1 |
| 16 | NUP93 | nudeoporin 93 | 102900 | 187193 | 070915 | 10, 24, 3 |
| 16 | NOD2 | nucleotide binding oligomerization domain containing 2 | 167207 | 167208 | 083799 | 4, 36, 20 |
| 16 | NUBP1 | nucleotide binding protein 1 | 103274 | 153060 | 166676 | 15, 34, 35 |
| 16 | NUBP2 | nucleotide binding protein 2 | 095906 | 162032 | 099769 | 1, 34, 27 |
| 16 | NDE1 | nudE neurodevelopment protein 1 | 072864 | 166783 | 133392 | 27, 23, 39 |
| 16 | NUDT16L1 | nudix hydrolase 16 like 1 | 168101 | 102858 | 168096 | 21, 33, 34 |
| 16 | NUDT21 | nudix hydrolase 21 | 167005 | 159461 | 087263 | 23, 40, 22 |
| 16 | NUDT7 | nudix hydrolase 7 | 140876 | 140873 | 171724 | 26, 17, 7 |
| 16 | OR1F1 | olfactory receptor family 1 subfamily F member 1 | 168124 | 085644 | 010539 | |
| 16 | OR2C1 | olfactory receptor family 2 subfamily C member 1 | 168158 | 162086 | 262621 | 32, |
| 16 | ORAI3 | ORAI calcium release-activated calcium modulator 3 | 175938 | 099364 | 099381 | 9, 17, 26 |
| 16 | ORC6 | origin recognition complex subunit 6 | 091651 | 069329 | 140795 | 43, 12, 13 |
| 16 | OTOA | otoancorin | 155719 | 140749 | 185864 | 21, 38, 10 |
| 16 | OSGIN1 | oxidative stress induced growth inhibitor 1 | 140961 | 260300 | 103154 | 33, 34, 29 |
| 16 | PARD6A | par-6 family cell polarity regulator alpha | 102981 | 102977 | 124074 | 29, 21, 26 |
| 16 | PALB2 | partner and localizer of BRCA2 | 083093 | 004779 | 166847 | 22, 12, 6 |
| 16 | PAGR1 | PAXIP1 associated glutamate rich protein 1 | 280789 | 280893 | 281348 | 9, 28, 31 |
| 16 | PDZD9 | PDZ domain containing 9 | 155714 | 140740 | 185716 | 38, 36, 8 |
| 16 | PTX4 | pentraxin 4 | 251692 | 103249 | 100726 | |
| 16 | PDF | peptide deformylase (mitochondrial) | 258429 | 272617 | 260371 | |
| 16 | PPL | periplakin | 118898 | 118900 | 103184 | 18, 23, 1 |
| 16 | PHLPP2 | PH domain and leucine rich repeat protein phosphatase 2 | 040199 | 140832 | 166747 | 22, 31, 38 |
| 16 | PIGQ | phosphatidylinositol glycan anchor biosynthesis class Q | 007541 | 161992 | 257108 | 26, 9, 31 |
| 16 | PGP | phosphoglycolate phosphatase | 184207 | 182685 | 167967 | 9, 20, 38 |
| 16 | PLA2G10 | phosph olipase A2 group X | 069764 | 103429 | 224712 | 3, 20, 28 |
| 16 | PLA2G15 | phospholipase A2 group XV | 103066 | 103067 | 103064 | 36, 18, 2 |
| 16 | PLCG2 | phospholipase C gamma 2 | 197943 | 153815 | 184860 | 21, 22, 43 |
| 16 | PMM2 | phosphomannomutase 2 | 140650 | 184857 | 153048 | 45, 24, 16 |
| 16 | PHKG2 | phosphorylase kinase catalytic subunit gamma 2 | 156873 | 281991 | 196118 | |
| 16 | PHKB | phosphorylase kinase regulatory subunit beta | 102893 | 129636 | 140798 | 7, 26, 17 |
| 16 | PIEZO1 | piezo type mechanosensitive ion channel component 1 | 103335 | 174177 | 167513 | 21, 6, 4 |
| 16 | PLLP | plasmolipin | 102934 | 102931 | 102962 | 26, 29, 37 |
| 16 | PLEKHG4 | pleckstrin homology and RhoGEF domain containing G4 | 196155 | 135740 | 168676 | 38, 5, 18 |
| 16 | PLK1 | polo like kinase 1 | 166851 | 166847 | 134398 | 3, 21, 41 |
| 16 | PABPN1L | poly(A) binding protein nuclear 1 like, cytoplasmic | 205022 | 167515 | 129993 | |
| 16 | PAPD5 | poly(A) RNA polymerase D5, non-canonical | 121274 | 155393 | 121281 | 45, 19, 31 |
| 16 | PARN | poly(A)-specific ribonuclease | 140694 | 186260 | 103429 | 45, 24, 39 |
| 16 | PMFBP1 | polyamine modulated factor 1 binding protein 1 | 118557 | 140829 | 280264 | 38, 5, 15 |
| 16 | PKD1L2 | polycystin 1 like 2 (gene/pseudogene) | 166473 | 140905 | 279362 | 1, 22, 8 |
| 16 | PKD1L3 | polycystin 1 like 3, transient receptor potential channel interacting | 277481 | 102984 | 102967 | 14, |
| 16 | PKD1 | polycystin 1, transient receptor potential channel interacting | 008710 | 103197 | 167964 | 36, 8, 22 |
| 16 | KCTD13 | potassium channel tetramerization domain containing 13 | 174943 | 174939 | 149932 | 11, 4, 38 |
| 16 | KCTD19 | potassium channel tetramerization domain containing 19 | 168676 | 196155 | 159708 | 38, 5, 14 |
| 16 | KCTD5 | potassium channel tetramerization domain containing 5 | 167977 | 140992 | 172382 | 22, 24, 44 |
| 16 | KCNG4 | potassium voltage-gated channel modifier subfamily G member 4 | 168418 | 140955 | 103175 | 25, 21, |
| 16 | PRDM7 | PR/SET domain 7 | 126856 | 283182 | 198156 | 35, 17, |
| 16 | PAM16 | presequence translocase associated motor 16 homolog | 217930 | 126603 | 103426 | 3, 15, |
| 16 | PAQR4 | progestin and adipoQ receptor family member 4 | 162073 | 127564 | 270168 | 9, 21, 14 |
| 16 | PRR14 | proline rich 14 | 156858 | 156853 | 156860 | 41, 35, 13 |
| 16 | PRR25 | proline rich 25 | 167945 | 127588 | 103227 | 14, 38, |
| 16 | PRR35 | proline rich 35 | 161992 | 103326 | 007541 | 14, 38, |
| 16 | PRRT2 | proline rich transmembrane protein 2 | 167371 | 103495 | 280893 | 8, 43, 2 |
| 16 | PRM1 | protamine 1 | 175646 | 122304 | 280717 | 38, 32, 8 |
| 16 | PRM2 | protamine 2 | 122304 | 178257 | 175646 | 38, 32, 8 |
| 16 | PRM3 | protamine 3 | 178257 | 178279 | 122304 | |
| 16 | PRSS21 | protease, serine 21 | 007038 | 103355 | 162078 | 9, 38, 18 |
| 16 | PRSS22 | protease, serine 22 | 005001 | 162078 | 162076 | 37, 13, 42 |
| 16 | PRSS27 | protease, serine 27 | 172382 | 167977 | 167978 | 13, 30, 9 |
| 16 | PRSS33 | protease, serine 33 | 103355 | 103363 | 007038 | 35, 5, 44 |
| 16 | PRSS36 | protease, serine 36 | 178226 | 052344 | 089280 | 9, 39, 10 |
| 16 | PRSS53 | protease, serine 53 | 151006 | 167395 | 255439 | 6, 14, 37 |
| 16 | PRSS54 | protease, serine 54 | 103023 | 103021 | 181938 | 38, 10, 8 |
| 16 | PRSS8 | protease, serine 8 | 052344 | 103510 | 178226 | 13, 4, 16 |
| 16 | PSMD7 | proteasome 26S subunit, nen-ATPase 7 | 103035 | 140836 | 196436 | 3, 6, 11 |
| 16 | PSMB10 | proteasome subunit beta 10 | 205220 | 261884 | 213398 | 41, 22, 3 |
| 16 | PRMT7 | protein arginine methyltransferase 7 | 132600 | 103061 | 103056 | 39, 36, 45 |
| 16 | PDIA2 | protein disulfide isomerase family A member 2 | 185615 | 242173 | 103126 | 4, 28, 8 |
| 16 | PDILT | protein disulfide isomerase like, testis expressed | 169340 | 169344 | 183549 | 38, |
| 16 | PRKCB | protein kinase C beta | 166501 | 166869 | 006116 | 22, 39, 36 |
| 16 | PKMYT1 | protein kinase, membrane associated tyrosine/threonine 1 | 127564 | 131650 | 162073 | |
| 16 | PPP4C | protein phosphatase 4 catalytic subunit | 149923 | 149925 | 149922 | 12, 27, 6 |
| 16 | PSKH1 | protein serine kinase H1 | 159792 | 188038 | 141086 | 40, 29, 26 |
| 16 | PYCARD | PYD and CARD domain containing | 103490 | 089280 | 177238 | 43, 41, 16 |
| 16 | PDXDC1 | pyridpxal dependent decarboxylase domain containing 1 | 179889 | 183426 | 157045 | 42, 38, 24 |
| 16 | PYDC1 | pyrin domain containing 1 | 169900 | 177238 | 169896 | 15, 10, 8 |
| 16 | PDPR | pyruvate dehydrogenase phosphatase regulatory subunit | 090857 | 279533 | 157335 | 40, 22, 38 |
| 16 | PDP2 | pyruvate dehyrogenase phosphatase catalytic subunit 2 | 172840 | 168748 | 166589 | 13, 8, 3 |
| 16 | QPRT | quinolinate phosphoribosyltransferase | 103485 | 277669 | 185905 | 27, |
| 16 | RAB11FIP3 | RAB11 family interacting protein 3 | 090565 | 242612 | 103326 | 34, 17, 6 |
| 16 | RAB26 | RAB26, member RAS oncogene family | 167964 | 008710 | 131653 | 33, 19, 28 |
| 16 | RAB40C | RAB40C, member RAS oncogene family | 197562 | 257108 | 127578 | 33, 9, 20 |
| 16 | RABEP2 | rabaptin, RAB GTPase binding effector protein 2 | 177548 | 196296 | 177455 | 36, 24, 42 |
| 16 | RANBP10 | RAN binding protein 10 | 141084 | 141098 | 102904 | 40, 26, 3 |
| 16 | RHOT2 | ras homolog family member T2 | 140983 | 161996 | 103269 | 5, 6, 22 |
| 16 | RBBP6 | RB binding protein 6, ubiquitin ligase | 122257 | 006116 | 090905 | 32, 29, 22 |
| 16 | RBL2 | RB transcriptional corepressor like 2 | 103479 | 279018 | 166971 | 22, 4, 39 |
| 16 | RMI2 | RecQ mediated genome instability 2 | 175643 | 038532 | 185338 | |
| 16 | RGS11 | regulator of G-protein signaling 11 | 076344 | 269881 | 242173 | 40, 26, 8 |
| 16 | ARHGDIG | Rhe GDP dissociation inhibitor gamma | 242173 | 076344 | 185615 | 14, 8, 44 |
| 16 | ARHGAP17 | Rho GTPase activating protein 17 | 140750 | 158865 | 205629 | 40, 32, 2 |
| 16 | RHBDF1 | rhomboid 5 homolog 1 | 007384 | 161981 | 103152 | 11, 33, 29 |
| 16 | RHBDL1 | rhomboid like 1 | 103269 | 140983 | 103266 | 14, 9, 39 |
| 16 | RSL1D1 | ribosomal L1 domain containing 1 | 171490 | 122299 | 103342 | 34, 4, 7 |
| 16 | RPL13 | ribosomal protein L13 | 167526 | 197912 | 178773 | 41, 21, 22 |
| 16 | RPL3L | ribosomal protein L3 like | 140986 | 198736 | 140990 | 25, 15, 26 |
| 16 | RPS15A | ribosomal protein S15a | 134419 | 185164 | 260342 | 12, 43, 41 |
| 16 | RPS2 | ribosomal protein S2 | 140988 | 140990 | 179580 | |
| 16 | RSPRY1 | ring finger and SPRY domain containing 1 | 159579 | 172775 | 102931 | 42, 30, 6 |
| 16 | RFWD3 | ring finger and WD repeat domain 3 | 168411 | 090863 | 168404 | 41, 42, 38 |
| 16 | RNF151 | ring finger protein 151 | 179580 | 140988 | 183751 | 38, |
| 16 | RNF166 | ring finger protein 166 | 158717 | 280603 | 174177 | 39, 5, 36 |
| 16 | RNF40 | ring finger protein 40 | 103549 | 196118 | 102870 | 43, 42, 38 |
| 16 | RNPS1 | RNA binding protein with serine rich domain 1 | 205937 | 167969 | 279473 | 3, 11, 33 |
| 16 | RBFOX1 | RNA binding protein, fox-1 homolog 1 | 078328 | 118894 | 232258 | 25, 8, 1 |
| 16 | POLR2C | RNA polymerase II subunit C | 102978 | 088682 | 125170 | 31, 34, 41 |
| 16 | POLR3E | RNA polymerase III subunit E | 058600 | 103319 | 140743 | 24, 22, 40 |
| 16 | POLR3K | RNA polymerase III subunit K | 161980 | 198156 | 161981 | 14, 28, 3 |
| 16 | RPUSD1 | RNA pseudouridylate synthase domain containing 1 | 007376 | 162006 | 127586 | 9, 33, 41 |
| 16 | ROGDI | rogdi homolog | 067836 | 267795 | 140632 | 8, 14, 26 |
| 16 | RPGRIP1L | RPGRIP1 like | 103494 | 166971 | 140718 | 38, 31, 42 |
| 16 | RRAD | RRAD, Ras related glycolysis inhibitor and calcium channel regulator | 166592 | 166589 | 166595 | 18, 25, 15 |
| 16 | RRN3 | RRN3 homolog, RNA polymerase I transcription factor | 085721 | 157045 | 183793 | 34, 31, 9 |
| 16 | SGF29 | SAGA complex associated factor 29 | 176476 | 176046 | 197165 | 1, 36, 15 |
| 16 | SRL | sarcalumenin | 185739 | 162104 | 090447 | 25, 24, 15 |
| 16 | SEC14L5 | SEC14 like lipid binding 5 | 103184 | 118898 | 103174 | 11, 8, 23 |
| 16 | SEZ6L2 | seizure related 6 homolog like 2 | 174938 | 103502 | 174939 | 26, 14, 28 |
| 16 | SEPHS2 | selenophosphate synthetase 2 | 179918 | 179958 | 005844 | 19, 17, 5 |
| 16 | SEPT1 | septin 1 | 180096 | 180035 | 283620 | 39, 22, 5 |
| 16 | SEPT12 | septin 12 | 140623 | 103199 | 267795 | 38, 8, 44 |
| 16 | SRRM2 | serine/arginine repetitive matrix 2 | 167978 | 172382 | 103363 | |
| 16 | SETD1A | SET domain containing 1A | 099381 | 175938 | 099377 | 41, 23, 22 |
| 16 | SETD6 | SET domain containing 6 | 103037 | 103034 | 125107 | 31, 24, 35 |
| 16 | SH2B1 | SH2B adaptor protein 1 | 178188 | 178952 | 196296 | 34, 26, 31 |
| 16 | SBK1 | SH3 domain binding kinase 1 | 188322 | 169180 | 198156 | 14, 26, 12 |
| 16 | SHCBP1 | SHC binding and spindle associated 1 | 171241 | 259680 | 069329 | 11, 39, 12 |
| 16 | SHISA9 | shisa family member 9 | 237515 | 103381 | 175595 | 18, 14, 8 |
| 16 | SDR42E1 | short chain dehydrogenase/reductase family 42E, member 1 | 184860 | 197943 | 086696 | 22, 4, 23 |
| 16 | SDR42E2 | short chain dehydrogenase/reductase family 42E, member 2 | 183921 | 175267 | 103319 | |
| 16 | SIAH1 | siah E3 ubiquitin protein ligase 1 | 196470 | 102910 | 102921 | 11, 32, 13 |
| 16 | SPN | sialophorin | 197471 | 169203 | 277669 | 39, 36, 5 |
| 16 | SLC9A3R2 | SLC9A3 regulator 2 | 065054 | 183971 | 065057 | 3, 1, 33 |
| 16 | SLX1A | SLX1 homolog A, structure-specific endonuclease subunit | 132207 | 169627 | 261052 | 13, 5, 22 |
| 16 | SLX1B | SLX1 homolog B, structure-specific endonuclease subunit | 181625 | 261740 | 213648 | 41, 6, 35 |
| 16 | SLX4 | SLX4 structure-specific endonuclease subunit | 188827 | 167984 | 213918 | 22, 24, 3 |
| 16 | SMIM22 | small integral membrane protein 22 | 267795 | 140623 | 067836 | 18, 16, 38 |
| 16 | SNRNP25 | small nuclear ribonucleoprotein U11/U12 subunit 25 | 161981 | 161980 | 007384 | 3, 21, 6 |
| 16 | SMG1 | SMG1, nonsense mediated mRNA decay associated PI3K related kinase | 157106 | 170540 | 170537 | |
| 16 | SNAI3 | snail family transcriptional repressor 3 | 185669 | 167508 | 280603 | |
| 16 | SRCAP | Snf2 related CREBBP activator protein | 080603 | 156860 | 282034 | 18, 34, 16 |
| 16 | SCNN1B | sodium channel epithelial 1 beta subunit | 168447 | 166828 | 168434 | 24, 22, 17 |
| 16 | SCNN1G | sodium channel epithelial 1 gamma subunit | 166828 | 103404 | 168447 | 26, 17, 44 |
| 16 | SLC12A3 | solute carrier family 12 member 3 | 070915 | 102900 | 051108 | 17, 9, 32 |
| 16 | SLC12A4 | solute carrier family 12 member 4 | 124067 | 213398 | 141096 | 32, 39, 23 |
| 16 | SLC22A31 | solute carrier family 22 member 31 | 259803 | 129910 | 170100 | 33, 12, 23 |
| 16 | SLC38A7 | solute carrier family 38 member 7 | 103042 | 279816 | 125166 | 45, 24, 38 |
| 16 | SLC38A8 | solute carrier family 38 member 8 | 166558 | 103154 | 140943 | 38, |
| 16 | SLC5A11 | solute carrier family 5 member 11 | 158865 | 090905 | 140750 | 10, 17, 29 |
| 16 | SLC5A2 | solute carrier family 5 member 2 | 140675 | 140682 | 140688 | 17, 38, 28 |
| 16 | SLC6A2 | solute carrier family 6 member 2 | 103546 | 256812 | 198848 | 26, 18, 32 |
| 16 | SLC7A5 | solute carrier family 7 member 5 | 103257 | 104731 | 174990 | 21, 9, 35 |
| 16 | SLC7A6 | solute carrier family 7 member 6 | 103064 | 103066 | 103061 | 18, 39, 22 |
| 16 | SLC7A6OS | solute carrier family 7 member 6 opposite strand | 103061 | 103064 | 132600 | 42, 39, 43 |
| 16 | SLC9A5 | solute carrier family 9 member A5 | 135740 | 135723 | 196155 | 23, 36, 40 |
| 16 | SSTR5 | somatostatin receptor 5 | 162009 | 005513 | 184471 | 28, 10, 42 |
| 16 | SNX20 | sorting nexin 20 | 167208 | 140807 | 167207 | 22, 39, 7 |
| 16 | SNX29 | sorting nexin 29 | 048471 | 048462 | 103381 | 22, 41, 21 |
| 16 | SALL1 | spalt like transcription factor 1 | 103449 | 083799 | 103460 | 12, 17, 8 |
| 16 | SPATA2L | spermatogenesis associated 2 like | 158792 | 185324 | 075399 | 22, 33, 13 |
| 16 | SPATA33 | spermatogenesis associated 33 | 167523 | 131165 | 185324 | 26, 38, 3 |
| 16 | SPG7 | SPG7, paraplegin matrix AAA peptidase subunit | 197912 | 167522 | 167526 | 26, 18, 27 |
| 16 | SPNS1 | sphingolipid transporter 1 (putative) | 169682 | 176953 | 213658 | 5, 9, 35 |
| 16 | SMPD3 | sphingomyelin phosphodiesterase 3 | 103056 | 132600 | 184939 | 39, 1, 26 |
| 16 | SPIRE2 | spire type actin nucleation factor 2 | 204991 | 187741 | 141002 | |
| 16 | SPSB3 | splA/ryanodine receptor domain and SOCS box containing 3 | 162032 | 197774 | 095906 | 28, 22, 26 |
| 16 | SF3B3 | splicing factor 3b subunit 3 | 189091 | 103051 | 157368 | 43, 21, 39 |
| 16 | SOX8 | SRY-box 8 | 005513 | 103227 | 162009 | 8, 26, 14 |
| 16 | ST3GAL2 | ST3 beta-galactoside alpha-2,3-sialyltransferase 2 | 157350 | 168872 | 157353 | 22, 29, 21 |
| 16 | SNN | stannin | 184602 | 189067 | 153066 | 34, 43, 11 |
| 16 | STUB1 | STIP1 homology and U-box containing protein 1 | 103266 | 103269 | 161999 | 34, 35, 26 |
| 16 | SULT1A1 | sulfotransferase family 1A member 1 | 196502 | 197165 | 255524 | 40, 19, 6 |
| 16 | SULT1A2 | sulfotransferase family 1A member 2 | 197165 | 176476 | 196502 | 15, 40, 22 |
| 16 | SULT1A3 | sulfotransferase family 1A member 3 | 261052 | 132207 | 258130 | 13, 26, 15 |
| 16 | SULT1A4 | sulfotransferase family 1A member 4 | 213648 | 181625 | 258150 | 9, 1, 43 |
| 16 | SOCS1 | suppressor of cytokine signaling 1 | 185338 | 175643 | 178279 | 27, 22, 15 |
| 16 | SYNGR3 | synaptogyrin 3 | 127561 | 127554 | 167962 | 26, 41, 8 |
| 16 | SYCE1L | synaptonemal complex central element protein 1 like | 205078 | 103111 | 140873 | 17, 12, 33 |
| 16 | SYT17 | synaptotagmin 17 | 103528 | 261427 | 261210 | 31, 26, 41 |
| 16 | STX1B | syntaxin 1B | 099365 | 099377 | 103496 | 8, 14, 12 |
| 16 | STX4 | syntaxin 4 | 103496 | 099365 | 167394 | 18, 6, 20 |
| 16 | SNTB2 | syntrophin beta 2 | 168807 | 141076 | 260914 | 22, 29, 11 |
| 16 | TAOK2 | TAO kinase 2 | 149930 | 149932 | 149929 | 34, 26, 2 |
| 16 | TAF1C | TATA-box binding protein associated factor, RNA polymerase I subunit C | 103168 | 154099 | 140955 | 40, 26, 9 |
| 16 | TLDC1 | TBC/LysM-associated domain containing 1 | 140950 | 064270 | 103187 | 9, 18, 23 |
| 16 | TBC1D10B | TBC1 domain family member 10B | 169221 | 169217 | 180209 | 34, 27, 41 |
| 16 | TBC1D24 | TBC1 domain family member 24 | 162065 | 162068 | 260272 | |
| 16 | TBX6 | T-box 6 | 149922 | 149923 | 090238 | 18, 21, 6 |
| 16 | TEKT5 | tektin 5 | 153060 | 213853 | 103274 | 38, 35, 44 |
| 16 | TELO2 | telomere maintenance 2 | 100726 | 251692 | 187535 | 9, 3, 41 |
| 16 | TERB1 | telomere repeat binding bouquet formation protein 1 | 249961 | 135720 | 159593 | 38, |
| 16 | TERF2 | telomeric repeat binding factor 2 | 132604 | 157315 | 103018 | 5, 18, 1 |
| 16 | TERF2IP | TERF2 interacting protein | 166848 | 065427 | 152910 | 31, 11, 8 |
| 16 | TEPP | testis, prostate and placenta expressed | 159648 | 070729 | 166188 | 38, 33, |
| 16 | THAP11 | THAP domain containing 11 | 168286 | 102901 | 102898 | 27, 20, 29 |
| 16 | TXNDC11 | thioredoxin domain containing 11 | 153066 | 184602 | 122299 | 7, 24, 10 |
| 16 | TXNL4B | thioredoxin like 4B | 140830 | 102967 | 257017 | 36, 17, 22 |
| 16 | THOC6 | THO complex 6 | 131652 | 103145 | 162069 | 41, 21, 27 |
| 16 | THUMPD1 | THUMP domain containing 1 | 066654 | 166743 | 196678 | 45, 30, 43 |
| 16 | TK2 | thymidine kinase 2, mitochondrial | 166548 | 260851 | 217555 | 26, 9, 1 |
| 16 | TIGD7 | tigger transposable element derived 7 | 140993 | 006194 | 162086 | 29, 38, 42 |
| 16 | TRAF7 | TNF receptor associated factor 7 | 131653 | 167964 | 167971 | 21, 6, 27 |
| 16 | TRAP1 | TNF receptor associated protein 1 | 126602 | 213918 | 005339 | 21, 3, 9 |
| 16 | TNFRSF12A | TNF receptor superfamily member 12A | 006327 | 184697 | 103145 | 37, 34, 35 |
| 16 | TNFRSF17 | TNF receptor superfamily member 17 | 048462 | 234719 | 048471 | 30, 22, 23 |
| 16 | TRADD | TNFRSF1A associated via death domain | 102871 | 237172 | 135722 | 29, 22, 36 |
| 16 | TOX3 | TOX high mobility group box family member 3 | 103460 | 103449 | 277639 | 11, 26, 37 |
| 16 | TP53TG3 | TP53 target 3 | 183632 | 205456 | 259303 | 38, 7, 32 |
| 16 | TP53TG3E | TP53 target 3 family member E | 275034 | 205457 | 261509 | 32, 38, |
| 16 | TP53TG3F | TP53 target 3 family member F | 278848 | 261509 | 270467 | |
| 16 | TP53TG3B | TP53 target 3B | 261509 | 275034 | 278848 | 38, 7, 32 |
| 16 | TP53TG3C | TP53 target 3C | 205457 | 271130 | 275034 | 32, 38, |
| 16 | TP53TG3D | TP53 target 3D | 205456 | 270472 | 183632 | 38, 4, 35 |
| 16 | TRAPPC2L | trafficking protein particle complex 2 like | 167515 | 280675 | 205022 | 2, 43, 29 |
| 16 | TCEB2 | Transcription elongation factor B subunit 2 | 103363 | 167978 | 103355 | 29, 25, 11 |
| 16 | TCF25 | transcription factor 25 | 141002 | 204991 | 258839 | 1, 32, 21 |
| 16 | TFAP4 | transcription factor AP-4 | 090447 | 185739 | 126603 | 32, 16, 3 |
| 16 | TBL3 | transducin beta like 3 | 183751 | 179580 | 196408 | 3, 21, 22 |
| 16 | TGFB1I1 | transforming growth factor beta 1 induced transcript 1 | 140682 | 140691 | 140675 | 43, 44, 13 |
| 16 | TVP23A | trans-golgi network vesicle protein 23 homolog A | 166676 | 103274 | 179583 | 1, 30, 18 |
| 16 | TNP2 | transition protein 2 | 178279 | 185338 | 178257 | 38, |
| 16 | TSNAXIP1 | translin associated factor X interacting protein 1 | 102904 | 141084 | 102901 | 38, 42, 6 |
| 16 | TMC5 | transmembrane channel like 5 | 103534 | 261210 | 006007 | 42, 37, 16 |
| 16 | TMC7 | transmembrane channel like 7 | 170537 | 157106 | 167186 | 38, 5, 8 |
| 16 | TMED6 | transmembrane p24 trafficking protein 6 | 157315 | 132603 | 132604 | 28, 37, 19 |
| 16 | TMEM114 | transmembrane protein 114 | 232258 | 078328 | 067365 | 20, 14, 33 |
| 16 | TMEM159 | transmembrane protein 159 | 011638 | 158486 | 103310 | 25, 28, 22 |
| 16 | TMEM170A | transmembrane protein 170A | 166822 | 261717 | 183196 | 10, 33, 1 |
| 16 | TMEM186 | transmembrane protein 186 | 184857 | 183044 | 140650 | 37, 33, 30 |
| 16 | TMEM204 | transmembrane protein 204 | 131634 | 187535 | 007545 | 2, 36, 15 |
| 16 | TMEM208 | transmembrane protein 208 | 168701 | 125122 | 135723 | 3, 40, 33 |
| 16 | TMEM219 | transmembrane protein 219 | 149932 | 174943 | 149930 | 38, 1, 43 |
| 16 | TMEM231 | transmembrane protein 231 | 205084 | 135702 | 034713 | 42, 11, 38 |
| 16 | TMEM265 | transmembrane protein 265 | 281991 | 282034 | 156873 | |
| 16 | TMEM8A | transmembrane protein 8A | 129925 | 086504 | 103202 | 26, 20, 16 |
| 16 | TANGO6 | transport and golgi organization 6 homolog | 103047 | 039068 | 103044 | 26, 25, 24 |
| 16 | TNRC6A | trinucleotide repeat containing 6A | 090905 | 122257 | 158865 | 18, 11, 40 |
| 16 | TRIM72 | tripartite motif containing 72 | 177238 | 103490 | 169900 | 15, 24, 25 |
| 16 | TPSAB1 | tryptase alpha/beta 1 | 172236 | 197253 | 095917 | |
| 16 | TPSB2 | tryptase beta 2 (gene/pseudogene) | 197253 | 116176 | 172236 | 1, 26, 37 |
| 16 | TPSD1 | tryptase delta 1 | 095917 | 172236 | 103275 | 25, 14, |
| 16 | TPSG1 | tryptase gamma 1 | 116176 | 196557 | 197253 | 16, 17, 14 |
| 16 | TSR3 | TSR3, acp transferase ribosome maturation factor | 007520 | 007516 | 090581 | 27, 22, 20 |
| 16 | TUFM | Tu translation elongation factor, mitochondrial | 178952 | 168488 | 178188 | 3, 21, 35 |
| 16 | TSC2 | tuberous sclerosis 2 | 103197 | 065057 | 008710 | 21, 18, 39 |
| 16 | TUBB3 | tubulin beta 3 class III | 258947 | 198211 | 140995 | 26, 8, 34 |
| 16 | TPPP3 | tubulin polymerization promoting protein family member 3 | 159713 | 159708 | 159714 | 18, 26, 1 |
| 16 | TAT | tyrosine aminotransferase | 198650 | 140835 | 140832 | 19, 23, 25 |
| 16 | USB1 | U6 snRNA biogenesis phosphodiesterase 1 | 103005 | 166188 | 102996 | 45, 34, 43 |
| 16 | UBALD1 | UBA like domain containing 1 | 153443 | 205832 | 102858 | 34, 27, 3 |
| 16 | UBN1 | ubinuclein 1 | 118900 | 140632 | 118898 | 43, 26, 18 |
| 16 | UQCRC2 | ubiquinol-cytochrome c reductase core protein II | 140740 | 185864 | 155714 | 15, 30, 43 |
| 16 | UBE2I | ubiquitin conjugating enzyme E2 I | 103275 | 095917 | 007516 | 37, 40, 22 |
| 16 | UBFD1 | ubiquitin family domain containing 1 | 103353 | 103356 | 004779 | 21, 22, 12 |
| 16 | USP10 | ubiquitin specific peptidase 10 | 103194 | 135686 | 103196 | 24, 13, 8 |
| 16 | USP31 | ubiquitin specific peptidase 31 | 103404 | 122254 | 166828 | 43, 18, 4 |
| 16 | USP7 | ubiquitin specific peptidase 7 | 187555 | 283516 | 182831 | 22, 41, 14 |
| 16 | B3GNT9 | UDP-GlcNAc: betaGal beta-1,3-N-acetylglucosaminyltransferase 9 | 237172 | 125149 | 102871 | 31, 26, 6 |
| 16 | UNKL | unkempt family like zinc finger | 059145 | 090581 | 174109 | 43, 31, 41 |
| 16 | UMOD | uromodulin | 169344 | 169347 | 169340 | 17, 4, 8 |
| 16 | UTP4 | UTP4, small subunit processome component | 141076 | 168802 | 168807 | 13, 9, 45 |
| 16 | VAC14 | Vac14, PIKFYVE complex component | 103043 | 132613 | 157423 | 21, 26, 9 |
| 16 | VPS4A | vacuolar protein sorting 4 homolog A | 132612 | 260914 | 213380 | 41, 27, 9 |
| 16 | VASN | vasorin | 168140 | 262246 | 103423 | 26, 6, 14 |
| 16 | VAT1L | vesicle amine transport 1 like | 171724 | 140876 | 166509 | 26, 34, 11 |
| 16 | VKORC1 | vitamin K epoxide reductase complex subunit 1 | 167397 | 255439 | 103507 | 28, 34, 6 |
| 16 | VWA3A | von Willebrand factor A domain containing 3A | 175267 | 185716 | 183921 | 42, 20, 30 |
| 16 | VPS35 | VPS35, retromer complex component | 069329 | 171241 | 091651 | 13, 38, 36 |
| 16 | VPS9D1 | VPS9 domain containing 1 | 075399 | 158792 | 158805 | 34, 6, 28 |
| 16 | WFDC1 | WAP four-disulfide core domain 1 | 103175 | 168418 | 064270 | 34, 35, 32 |
| 16 | WFIKKN1 | WAP, follistatin/kazal, immunoglobulin, kunitz and netrin domain containing 1 | 127578 | 197562 | 130731 | 9, 12, 38 |
| 16 | WDR24 | WD repeat domain 24 | 127580 | 161999 | 127585 | 27, 1, 38 |
| 16 | WDR59 | WD repeat domain 59 | 103091 | 103089 | 186187 | 14, 3, 12 |
| 16 | WDR90 | WD repeat domain 90 | 161996 | 172366 | 140983 | 22, 39, 3 |
| 16 | WWP2 | WW domain containing E3 ubiquitin protein ligase 2 | 198373 | 141101 | 157322 | 42, 43, 1 |
| 16 | WWOX | WW domain containing oxidoreductase | 186153 | 166509 | 178573 | 34, 17, 36 |
| 16 | XYLT1 | xylosyltransferase 1 | 103489 | 214967 | 214940 | 2, 43, 22 |
| 16 | YPEL3 | yippee like 3 | 090238 | 149922 | 102886 | 32, 23, 40 |
| 16 | ZFP1 | ZFP1 zinc finger protein | 184517 | 166816 | 168928 | 43, 8, 4 |
| 16 | ZFP90 | ZFP90 zinc finger protein | 184939 | 103056 | 062038 | |
| 16 | ZNRF1 | zinc and ring finger 1 | 186187 | 103091 | 166816 | 9, 20, 28 |
| 16 | ZSCAN10 | zinc finger and SCAN domain containing 10 | 130182 | 008517 | 122386 | 12, 38, |
| 16 | ZSCAN32 | zinc finger and SCAN domain containing 32 | 140987 | 232196 | 103343 | 12, 37, 23 |
| 16 | ZC3H18 | zinc finger CCCH-type containing 18 | 158545 | 179588 | 124391 | 24, 42, 39 |
| 16 | ZC3H7A | zinc finger CCCH-type containing 7A | 122299 | 153066 | 171490 | |
| 16 | ZCCHC14 | zinc finger CCHC-type containing 14 | 140948 | 140941 | 154118 | 29, 16, 6 |
| 16 | ZDHHC1 | zinc finger DHHC-type containing 1 | 159714 | 159713 | 176387 | 26, 3, 13 |
| 16 | ZDHHC7 | zinc finger DHHC-type containing 7 | 153786 | 103196 | 135709 | 16, 13, 10 |
| 16 | ZFHX3 | zinc finger homeobox 3 | 140836 | 280264 | 103035 | 11, 26, 14 |
| 16 | ZNF174 | zinc finger protein 174 | 103343 | 140987 | 167981 | 9, 24, 39 |
| 16 | ZNF19 | zinc finger protein 19 | 157429 | 261611 | 140835 | |
| 16 | ZNF200 | zinc finger protein 200 | 010539 | 168124 | 103313 | |
| 16 | ZNF205 | zinc finger protein 205 | 122386 | 130182 | 085644 | 13, 9, 35 |
| 16 | ZNF213 | zinc finger protein 213 | 085644 | 122386 | 168124 | 44, 36, 39 |
| 16 | ZNF23 | zinc finger protein 23 | 167377 | 172137 | 261611 | 4, 2, 23 |
| 16 | ZNF263 | zinc finger protein 263 | 006194 | 103313 | 140993 | 15, 4, 24 |
| 16 | ZNF267 | zinc finger protein 267 | 185947 | 197302 | 270472 | 7, 42, 39 |
| 16 | ZNF276 | zinc finger protein 276 | 158805 | 075399 | 187741 | 1, 3, 36 |
| 16 | ZNF319 | zinc finger protein 319 | 166188 | 159648 | 103005 | 40, 1, 31 |
| 16 | ZNF423 | zinc finger protein 423 | 102935 | 166152 | 205423 | 11, 1, 10 |
| 16 | ZNF469 | zinc finger protein 469 | 225614 | 172530 | 179588 | 6, 22, 40 |
| 16 | ZNF48 | zinc finger protein 48 | 180035 | 180209 | 180096 | 33, 35, 2 |
| 16 | ZNF48 | zinc finger protein 48 | 283620 | 180096 | 179965 | 33, 35, 2 |
| 16 | ZNF500 | zinc finger protein 500 | 103199 | 166246 | 140623 | 42, 1, 38 |
| 16 | ZNF597 | zinc finger protein 597 | 167981 | 103343 | 122390 | 22, 42, 30 |
| 16 | ZNF598 | zinc finger protein 598 | 167962 | 127561 | 183971 | 9, 3, 14 |
| 16 | ZNF629 | zinc finger protein 629 | 102870 | 103549 | 099385 | 29, 33, 44 |
| 16 | ZNF646 | zinc finger protein 646 | 167395 | 167394 | 151006 | 41, 21, 1 |
| 16 | ZNF668 | zinc finger protein 668 | 167394 | 103496 | 167395 | 29, 24, 7 |
| 16 | ZNF688 | zinc finger protein 688 | 229809 | 169951 | 197162 | 34, 33, 41 |
| 16 | ZNF689 | zinc finger protein 689 | 156853 | 197162 | 156858 | 21, 11, 38 |
| 16 | ZNF720 | zinc finger protein 720 | 197302 | 169877 | 185947 | 11, 4, 18 |
| 16 | ZNF747 | zinc finger protein 747 | 169955 | 261459 | 260869 | 41, 13, 4 |
| 16 | ZNF75A | zinc finger protein 75a | 162086 | 140993 | 168158 | 22, 30, 11 |
| 16 | ZNF764 | zinc finger protein 764 | 169951 | 260869 | 229809 | 29, 22, 35 |
| 16 | ZNF768 | zinc finger protein 768 | 169957 | 005844 | 261459 | 21, 1, 26 |
| 16 | ZNF771 | zinc finger protein 771 | 179965 | 283620 | 179958 | 20, 29, 25 |
| 16 | ZNF778 | zinc finger protein 778 | 170100 | 259803 | 167522 | 30, 7, 45 |
| 16 | ZNF785 | zinc finger protein 785 | 197162 | 229809 | 156853 | 42, 36, 44 |
| 16 | ZNF821 | zinc finger protein 821 | 102984 | 182149 | 277481 | 41, 26, 31 |
| 16 | ZNF843 | zinc finger protein 843 | 176723 | 156885 | 140691 | 27, 14, 12 |
| 16 | ZFPM1 | zinc finger protein, FOG family member 1 | 179588 | 225614 | 158545 | 20, 27, 17 |
| 16 | ZKSCAN2 | zinc finger with KRAB and SCAN domains 2 | 155592 | 103375 | 182601 | 4, 39, 18 |
| 16 | ZP2 | zona pellucida glycoprotein 2 | 103310 | 011638 | 175311 | 8, 44, 38 |
| 16 | ZG16 | zymogen granule protein 16 | 174992 | 185905 | 079616 | 16, 28, 37 |
| 16 | ZG16B | zymogen granule protein 16B | 162078 | 007038 | 005001 | 34, 42, 37 |
| 17 | CNP | 2′,3′-cyclic nucleotide 3′ phosphodiesterase | 173786 | 204815 | 168259 | 8, 35, 17 |
| 17 | OGFOD3 | 2-oxoglutarate and iron dependent oxygenase domain containing 3 | 181396 | 181408 | 169660 | 40, |
| 17 | NT5C | 5′,3′-nudeotidase, cytosolic | 125458 | 125449 | 189159 | 3, 41, 34 |
| 17 | NT5M | 5′,3′-nucleotidase, mitochondrial | 205309 | 141030 | 141026 | 3, 9, 25 |
| 17 | NT5C3B | 5′-nucleotidase, cytosolic IIIB | 141698 | 141756 | 161594 | 9, 25, 38 |
| 17 | AATK-AS1 | AATK antisense RNA 1 | 225180 | 181409 | 141577 | |
| 17 | ABHD15 | abhydrolase domain containing 15 | 168792 | 160551 | 167543 | 22, 1, 30 |
| 17 | ABI3 | ABI family member 3 | 108798 | 167083 | 173868 | 41, 5, 31 |
| 17 | ACACA | acetyl-CoA carboxylase alpha | 278540 | 275700 | 278505 | 26, 33, 43 |
| 17 | ASIC2 | acid sensing ion channel subunit 2 | 108684 | 141316 | 108691 | 31, 8, 14 |
| 17 | ACTG1 | actin gamma 1 | 184009 | 266074 | 186765 | |
| 17 | ABR | active BCR-related | 159842 | 177370 | 205899 | |
| 17 | ACBD4 | acyl-CoA binding domain containing 4 | 181513 | 161714 | 186834 | 34, 1, 14 |
| 17 | ACADVL | acyl-CoA dehydrogenase, very long chain | 072778 | 132535 | 004975 | 2, 1, 26 |
| 17 | ACOX1 | acyl-CoA oxidase 1 | 161533 | 188878 | 257949 | 18, 4, 29 |
| 17 | ACSF2 | acyl-CoA synthetase family member 2 | 167107 | 108829 | 136457 | 2, 17, 39 |
| 17 | ADAM11 | ADAM metallopeptidase domain 11 | 073670 | 161692 | 182963 | 14, 12, 8 |
| 17 | AP2B1 | adaptor related protein complex 2 beta 1 subunit | 006125 | 108733 | 270647 | 38, 43, 24 |
| 17 | ADORA2B | adenosine A2b receptor | 170425 | 214946 | 214941 | 24, 18, 15 |
| 17 | ARL16 | ADP ribosylation factor like GTPase 16 | 214087 | 185298 | 185359 | 34, 1, 12 |
| 17 | ARL17A | ADP ribosylation factor like GTPase 17A | 185829 | 238083 | 283194 | |
| 17 | ARL17B | ADP ribosylation factor like GTPase 17B | 228696 | 120071 | 176681 | 3, 39, 37 |
| 17 | ARL4D | ADP ribosylation factor like GTPase 4D | 175906 | 236383 | 067596 | 26, 14, 9 |
| 17 | ARL5C | ADP ribosylation factor like GTPase 5C | 141748 | 161381 | 067191 | 39, 15, |
| 17 | ADPRM | ADP-ribose/CDP-alcohol diphosphatase, manganese dependent | 170222 | 133028 | 187824 | 4, 26, 11 |
| 17 | AKAP1 | A-kinase anchoring protein 1 | 121057 | 121064 | 153944 | 38, 40, 37 |
| 17 | AKAP10 | A-kinase anchoring protein 10 | 108599 | 083290 | 128487 | 18, 45, 38 |
| 17 | AARSD1 | alanyl-tRNA synthetase domain containing 1 | 266967 | 131482 | 267060 | |
| 17 | ALDH3A1 | aldehyde dehydrogenase 3 family member A1 | 108602 | 180638 | 083290 | 13, 3, 14 |
| 17 | ALDH3A2 | aldehyde dehydrogenase 3 family member A2 | 072210 | 142494 | 180638 | 2, 42, 24 |
| 17 | ALDOC | aldolase, fructose-bisphosphate C | 109107 | 087111 | 076382 | 8, 1, 15 |
| 17 | ALKBH5 | alkB homolog 5, RNA demethylase | 091542 | 091536 | 131899 | 34, 1, 22 |
| 17 | ALYREF | Aly/REF export factor | 183684 | 141522 | 141552 | 22, 3, 34 |
| 17 | AOC2 | amine oxidase, copper containing 2 | 131480 | 131467 | 131471 | 14, 6, 10 |
| 17 | AOC3 | amine oxidase, copper containing 3 | 131471 | 131480 | 131482 | 44, 42, 1 |
| 17 | NPEPPS | aminopeptidase puromycin sensitive | 141279 | 178852 | 108424 | 24, 39, 23 |
| 17 | APPBP2 | amyloid beta precursor protein binding protein 2 | 062725 | 141371 | 170836 | 38, 37, 10 |
| 17 | ANAPC11 | anaphase promoting complex subunit 11 | 141552 | 183684 | 183979 | 29, 31, 34 |
| 17 | ACE | angiotensin I converting enzyme | 159640 | 008283 | 264813 | 38, 29, 1 |
| 17 | ANKFN1 | ankyrin repeat and fibronectin type III domain containing 1 | 153930 | 141179 | 183691 | |
| 17 | ANKFY1 | ankyrin repeat and FYVE domain containing 1 | 185722 | 167740 | 132388 | 24, 39, 36 |
| 17 | ASB16 | ankyrin repeat and SOCS box containing 16 | 161664 | 125319 | 168591 | 18, 4, 24 |
| 17 | ANKRD13B | ankyrin repeat domain 13B | 198720 | 108262 | 167549 | 9, 6, 7 |
| 17 | ANKRD40 | ankyrin repeat domain 40 | 154945 | 108846 | 108848 | 6, 40, 37 |
| 17 | APOH | apolipoprotein H | 091583 | 154240 | 154229 | 19, 28, 15 |
| 17 | AATF | apoptosis antagonizing transcription factor | 275700 | 273706 | 278540 | 10, 9, 3 |
| 17 | AATK | apoptosis associated tyrosine kinase | 181409 | 175866 | 225180 | 26, 8, 11 |
| 17 | ALOX12B | arachidonate 12-lipoxygenase, 12R type | 179477 | 179593 | 179148 | 35, 4, 30 |
| 17 | ALOX12 | arachidonate 12-lipoxygenase, 12S type | 108839 | 167858 | 219200 | 27, 25, 12 |
| 17 | ALOX15 | arachidonate 15-lipoxygenase | 161905 | 188176 | 141456 | 24, 42, 1 |
| 17 | ALOX15B | arachidonate 15-lipoxygenase, type B | 179593 | 132518 | 179477 | 33, 35, 1 |
| 17 | ALOXE3 | arachidonate lipoxygenase 3 | 179148 | 179477 | 179111 | 4, 30, 8 |
| 17 | AANAT | aralkylamine N-acetyltransferase | 129673 | 175931 | 129667 | 14, 27, 17 |
| 17 | AMZ2 | archaelysin family metallopeptidase 2 | 196704 | 182481 | 141337 | 34, 7, 38 |
| 17 | ACAP1 | ArfGAP with coiled-coil, ankyrin repeat and PH domains 1 | 072818 | 215041 | 213859 | 21, 5, 22 |
| 17 | ADAP2 | ArfGAP with dual PH domains 2 | 184060 | 172171 | 181481 | |
| 17 | JMJD6 | arginine demethylase and lysine hydroxylase | 070495 | 182534 | 181038 | 34, 3, 29 |
| 17 | ARMC7 | armadillo repeat containing 7 | 125449 | 170190 | 125458 | 3, 13, 34 |
| 17 | ARRB2 | arrestin beta 2 | 141480 | 141456 | 161920 | 5, 1, 9 |
| 17 | AIPL1 | aryl hydrocarbon receptor interacting protein like 1 | 129221 | 179314 | 129195 | 14, 25, 32 |
| 17 | AFMID | arylformamidase | 183077 | 167900 | 089685 | 18, 7, 27 |
| 17 | ARSG | arylsulfatase G | 141337 | 196704 | 108932 | 5, 30, 40 |
| 17 | ASGR1 | asialoglycoprotein receptor 1 | 141505 | 161944 | 132535 | 19, 21, 38 |
| 17 | ASGR2 | asialoglycoprotein receptor 2 | 161944 | 132514 | 141505 | 19, 5, 36 |
| 17 | ASPA | aspartoacylase | 108381 | 141255 | 167723 | 17, 2, 25 |
| 17 | ASPSCR1 | ASPSCR1, UBX domain containing tether for SLC2A4 | 169696 | 185269 | 169689 | 29, 19, 5 |
| 17 | ATXN7L3 | ataxin 7 like 3 | 087152 | 168591 | 108312 | 21, 39, 43 |
| 17 | ABCA10 | ATP binding cassette subfamily A member 10 | 154263 | 154262 | 154265 | 11, 42, 4 |
| 17 | ABCA5 | ATP binding cassette subfamily A member 5 | 154265 | 154263 | 108984 | 29, 31, 18 |
| 17 | ABCA6 | ATP binding cassette subfamily A member 6 | 154262 | 154258 | 154263 | 26, 45, 22 |
| 17 | ABCA8 | ATP binding cassette subfamily A member 8 | 141338 | 108950 | 154258 | 11, 26, 2 |
| 17 | ABCA9 | ATP binding cassette subfamily A member 9 | 154258 | 141338 | 154262 | 1, 25, 42 |
| 17 | ABCC3 | ATP binding cassette subfamily C member 3 | 108846 | 006283 | 154945 | 16, 2, 19 |
| 17 | ACLY | ATP citrate lyase | 131473 | 178502 | 204815 | |
| 17 | ATPAF2 | ATP synthase mitochondrial F1 complex assembly factor 2 | 171953 | 171962 | 141034 | 29, 3, 39 |
| 17 | ATP5G1 | ATP synthase, H+ transporting, mitochondrial Fp complex subunit C1 (subunit 9) | 159199 | 136436 | 159202 | 29, 15, 7 |
| 17 | ATP5H | ATP synthase, H+ transporting, mitpchpndrial Fp complex subunit D | 167863 | 281674 | 170190 | 43, 29, 27 |
| 17 | ATAD5 | ATPase family, AAA domain containing 5 | 176208 | 176390 | 172171 | 13, 7, 22 |
| 17 | ATP6V0A1 | ATPase H+ transporting V0 subunit a1 | 033627 | 177469 | 108784 | 8, 26, 39 |
| 17 | ATP1B2 | ATPase Na+/K+ transporting subunit beta 2 | 129244 | 141504 | 141510 | 8, 14, 26 |
| 17 | ATP2A3 | ATPase sarcoplasmic/endoplasmic reticulum Ca2+ transporting 3 | 074370 | 108405 | 074755 | 22, 28, 7 |
| 17 | AURKB | aurora kinase B | 178999 | 279152 | 178971 | 21, 19, 12 |
| 17 | AXIN2 | axin 2 | 168646 | 108370 | 266076 | 16, 44, 26 |
| 17 | B9D1 | B9 domain containing 1 | 108641 | 072134 | 166484 | 29, 31, 18 |
| 17 | BIRC5 | baculoviral IAP repeat containing 5 | 089685 | 183077 | 204278 | 34, 9, 39 |
| 17 | BAHCC1 | BAH domain and coiled-coil containing 1 | 266074 | 185332 | 184009 | |
| 17 | BAIAP2 | BAH associated protein 2 | 175866 | 176108 | 181409 | 8, 14, 9 |
| 17 | BHLHA9 | basic helix-loop-helix family member a9 | 205899 | 159842 | 184811 | |
| 17 | BCAS3 | BCAS3, microtubule associated cell migration factor | 141376 | 281267 | 121068 | |
| 17 | BCL6B | B-cell CLL/lymphoma 6B | 161940 | 258315 | 174327 | 44, 20, 17 |
| 17 | BECN1 | beclin 1 | 126581 | 176563 | 131467 | 45, 36, 15 |
| 17 | B4GALNT2 | beta-1,4-N-acetyl-galactosaminyltransferase 2 | 167080 | 159217 | 167083 | 15, 16, 38 |
| 17 | BLMH | bleomycin hydrolase | 108578 | 108576 | 182271 | 21, 45, 39 |
| 17 | BORCS6 | BLOC-1 related complex subunit 6 | 196544 | 179029 | 279152 | |
| 17 | BRIP1 | BRCA1 interacting protein C-terminal helicase 1 | 136492 | 253506 | 108506 | 38, 23, 22 |
| 17 | BRCA1 | BRCA1, DNA repair associated | 012048 | 108830 | 188554 | 22, 35, 3 |
| 17 | BPTF | bromodomain PHD finger transcription factor | 171634 | 130935 | 186665 | 26, 22, 18 |
| 17 | BTBD17 | BTB domain containing 17 | 204347 | 196169 | 257008 | 27, 8, 28 |
| 17 | C1QTNF1 | C1q and tumor necrosis factor related protein 1 | 173918 | 171302 | 167280 | 2, 26, 6 |
| 17 | CANT1 | calcium activated nucleotidase 1 | 171302 | 108679 | 173918 | |
| 17 | CALCOCO2 | calcium binding and coiled-coil domain 2 | 136436 | 170703 | 159199 | |
| 17 | CACNB1 | calcium voltage-gated channel auxiliary subunit beta 1 | 067191 | 141748 | 108298 | 26, 25, 15 |
| 17 | CACNG1 | calcium voltage-gated channel auxiliary subunit gamma 1 | 108878 | 075461 | 198265 | 25, 15, 17 |
| 17 | CACNG4 | calcium voltage-gated channel auxiliary subunit gamma 4 | 075461 | 075429 | 108878 | 26, 23, 8 |
| 17 | CACNG5 | calcium voltage-gated channel auxiliary subunit gamma 5 | 075429 | 154229 | 075461 | 27, 14, |
| 17 | CACNA1G | calcium voltage-gated channel subunit alpha1 G | 006283 | 006282 | 108846 | 11, 14, 15 |
| 17 | CAMKK1 | calcium/calmodulin dependent protein kinase kinase 1 | 004660 | 074356 | 108405 | 26, 9, 14 |
| 17 | CAMTA2 | calmodulin binding transcription activator 2 | 108509 | 091640 | 196388 | 21, 33, 14 |
| 17 | CASC3 | cancer susceptibility candidate 3 | 108349 | 188895 | 108352 | 42, 24, 38 |
| 17 | CA10 | carbonic anhydrase 10 | 154975 | 011260 | 141200 | 8, 14, 32 |
| 17 | CA4 | carbonic anhydrase 4 | 167434 | 068097 | 170832 | 1, 29, 15 |
| 17 | CPD | carboxypeptidase D | 108582 | 182271 | 108587 | 42, 1, 45 |
| 17 | CSNK1D | casein kinase 1 delta | 141551 | 141526 | 173762 | 24, 42, 39 |
| 17 | CASKIN2 | CASK interacting protein 2 | 177303 | 177728 | 182173 | 26, 18, 1 |
| 17 | CARD14 | caspase recruitment domain family member 14 | 141527 | 141543 | 181523 | 32, |
| 17 | CCL1 | C-C motif chemokine ligand 1 | 108702 | 181374 | 181291 | 41, 20, 38 |
| 17 | CCL11 | C-C motif chempkine ligand 11 | 172156 | 108688 | 108700 | 28, 44, 17 |
| 17 | CCL13 | C-C metif chempkine ligand 13 | 181374 | 108700 | 108702 | 28, 6, 20 |
| 17 | CCL14 | C-C metif chempkine ligand 14 | 276409 | 275152 | 275688 | 1, 36, 15 |
| 17 | CCL15 | C-C metif chempkine ligand 15 | 275718 | 275688 | 274736 | 11, 3, 17 |
| 17 | CCL16 | C-C metif chempkine ligand 16 | 275152 | 275722 | 276409 | 26, 19, |
| 17 | CCL18 | C-C metif chempkine ligand 18 | 275385 | 274736 | 277632 | 6, 28, 45 |
| 17 | CCL2 | C-C metif chemokine ligand 2 | 108691 | 108684 | 108688 | 2, 5, 28 |
| 17 | CCL23 | C-C motif chemokine ligand 23 | 274736 | 275718 | 275385 | 20, 19, 32 |
| 17 | CCL3 | C-C motif chemokine ligand 3 | 277632 | 275385 | 275302 | 5, 32, 14 |
| 17 | CCL3L3 | C-C motif chemokine ligand 3 like 3 | 276085 | 274808 | 276070 | 5, 29, 2 |
| 17 | CCL4 | C-C motif chemokine ligand 4 | 275302 | 277632 | 274808 | 5, 2, 29 |
| 17 | CCL4L2 | C-C motif chemokine ligand 4 like 2 | 276070 | 276085 | 274933 | 5, 2, 29 |
| 17 | CCL5 | C-C motif chemokine ligand 5 | 271503 | 270379 | 278023 | 30, 5, 1 |
| 17 | CCL7 | C-C motif chemokine ligand 7 | 108688 | 108691 | 172156 | 5, 35, 44 |
| 17 | CCL8 | C-C motif chemokine ligand 8 | 108700 | 172156 | 181374 | 2, 6, 19 |
| 17 | CCR10 | C-C motif chemokine receptor 10 | 184451 | 068137 | 108797 | 6, 14, 28 |
| 17 | CCR7 | C-C motif chemokine receptor 7 | 126353 | 131746 | 073584 | 30, 5, 36 |
| 17 | CCL15-CCL14 | CCL15-CCL14 readthrough (NMD candidate) | 275688 | 276409 | 275718 | |
| 17 | CD300LB | CD300 molecule like family member b | 178789 | 167851 | 167850 | 5, 36, 25 |
| 17 | CD300LD | CD300 molecule like family member d | 204345 | 167850 | 186407 | |
| 17 | CD300LF | CD300 molecule like family member f | 186074 | 172794 | 109062 | 7, 30, 34 |
| 17 | CD300LG | CD300 molecule like family member g | 161649 | 161647 | 108852 | 38, 32, 25 |
| 17 | CD300A | CD300a molecule | 167851 | 170412 | 178789 | 5, 11, 27 |
| 17 | CD300C | CD300c molecule | 167850 | 178789 | 204345 | 20, 42, 23 |
| 17 | CD300E | CD300e molecule | 186407 | 204345 | 172794 | |
| 17 | CD68 | CD68 molecule | 129226 | 161960 | 129255 | 1, 10, 36 |
| 17 | CD7 | CD7 molecule | 173762 | 141551 | 141574 | 5, 36, 41 |
| 17 | CD79B | CD79b molecule | 007312 | 259384 | 007314 | 41, 21, 22 |
| 17 | CDC42EP4 | CDC42 effector protein 4 | 179604 | 187959 | 069188 | 24, 28, 39 |
| 17 | CISD3 | CDGSH iron sulfur domain 3 | 277972 | 279119 | 277258 | 14, 27, 29 |
| 17 | CDK5RAP3 | CDK5 regulatory subunit associated protein 3 | 108465 | 167183 | 005243 | 39, 13, 36 |
| 17 | CDC27 | cell division cycle 27 | 004897 | 179673 | 198336 | 22, 10, 1 |
| 17 | CDC6 | cell division cycle 6 | 094804 | 171475 | 131759 | 9, 24, 45 |
| 17 | CNTROB | centrobin, centriole duplication and spindle assembly protein | 170037 | 170043 | 132518 | |
| 17 | CENPV | centromere protein V | 166582 | 108474 | 170315 | 37, 27, 12 |
| 17 | CENPX | centromere protein X | 169689 | 169696 | 169683 | |
| 17 | CEP112 | centrosomal protein 112 | 154240 | 266076 | 091583 | 38, 10, 13 |
| 17 | CEP131 | centrosomal protein 131 | 141577 | 225180 | 167302 | |
| 17 | CEP95 | centrosomal protein 95 | 258890 | 108654 | 108854 | 23, 7, 18 |
| 17 | CEP295NL | CEP295 N-terminal like | 178404 | 035862 | 108679 | |
| 17 | CDR2L | cerebellar degeneration related protein 2 like | 109089 | 167861 | 167862 | 26, 6, 3 |
| 17 | CCT6B | chaperonin containing TCP1 subunit 6B | 132141 | 181291 | 198783 | 38, 37, 45 |
| 17 | CHMP6 | charged multivesicular body protein 6 | 176108 | 141564 | 175866 | 34, 1, 9 |
| 17 | CHRNB1 | cholinergic receptor nicotinic beta 1 subunit | 170175 | 161958 | 174282 | 24, 25, 15 |
| 17 | CHRNE | cholinergic receptor nicotinic epsilon subunit | 108556 | 141503 | 205710 | 36, 14, |
| 17 | CHAD | chondroadherin | 136457 | 167107 | 136444 | 6, 10, 4 |
| 17 | CSH1 | chorionic somatomammotropin hormone 1 | 136488 | 136487 | 204414 | 32, 45, 11 |
| 17 | CSH2 | chorionic somatomammotropin hormone 2 | 213218 | 108604 | 136487 | 32, 45, 40 |
| 17 | CSHL1 | chorionic somatomammotropin hormone like 1 | 204414 | 136488 | 259384 | 32, 40, 5 |
| 17 | CBX1 | chromobox 1 | 108468 | 082641 | 002919 | 29, 11, 15 |
| 17 | CBX2 | chromobox 2 | 173894 | 182156 | 141570 | 12, 3, 44 |
| 17 | CBX4 | chromobox 4 | 141582 | 141570 | 167291 | 18, 9, 21 |
| 17 | CBX8 | chromobox 8 | 141570 | 173894 | 141582 | 13, 9, 18 |
| 17 | CHD3 | chromodomain helicase DNA binding protein 3 | 170004 | 182224 | 179859 | 18, 21, 22 |
| 17 | C17orf100 | chromosome 17 open reading frame 100 | 279257 | 108590 | 141485 | 20, 38, 16 |
| 17 | C17orf105 | chromosome 17 open reading frame 105 | 231256 | 108861 | 161647 | |
| 17 | C17orf107 | chromosome 17 open reading frame 107 | 205710 | 108556 | 185245 | 43, 42, 39 |
| 17 | C17orf47 | chromosome 17 open reading frame 47 | 181013 | 108387 | 121101 | 38, 5, |
| 17 | C17orf49 | chromosome 17 open reading frame 49 | 258315 | 161939 | 161940 | 22, 41, 7 |
| 17 | C17orf50 | chromosome 17 open reading frame 50 | 270806 | 271447 | 270379 | 38, 8, |
| 17 | C17orf51 | chromosome 17 open reading frame 51 | 212719 | 184185 | 260458 | |
| 17 | C17orf53 | chromosome 17 open reading frame 53 | 125319 | 108840 | 161664 | 41, 12, 22 |
| 17 | C17orf58 | chromosome 17 open reading frame 58 | 186665 | 171634 | 182481 | 9, 34, 18 |
| 17 | C17orf62 | chromosome 17 open reading frame 62 | 178927 | 169660 | 141562 | 36, 5, 21 |
| 17 | C17orf64 | chromosome 17 open reading frame 64 | 141371 | 170832 | 062725 | 38, 28, 10 |
| 17 | C17orf67 | chromosome 17 open reading frame 67 | 214226 | 183691 | 153933 | 39, 45, 28 |
| 17 | C17orf74 | chromosome 17 open reading frame 74 | 184560 | 181323 | 283439 | 38, 8, |
| 17 | C17orf75 | chromosome 17 open reading frame 75 | 108666 | 141314 | 010244 | 26, 38, 11 |
| 17 | C17orf78 | chromosome 17 open reading frame 78 | 278505 | 278540 | 276234 | 2, 16, |
| 17 | C17orf80 | chromosome 17 open reading frame 80 | 141219 | 133193 | 187959 | 29, 41, 22 |
| 17 | C17orf96 | Chromosome 17 open reading frame 96 | 273604 | 277363 | 275023 | 29, 3, 9 |
| 17 | C17orf97 | chromosome 17 open reading frame 97 | 187624 | 181031 | 183688 | 38, 31, 23 |
| 17 | C17orf98 | chromosome 17 open reading frame 98 | 275489 | 273559 | 125691 | 38, 10, |
| 17 | C17orf99 | chromosome 17 open reading frame 99 | 187997 | 167895 | 108639 | 39, 15, 36 |
| 17 | CFAP52 | cilia and flagella associated protein 52 | 166596 | 170310 | 154914 | 30, 42, 38 |
| 17 | CLTC | clathrin heavy chain | 141367 | 108406 | 141378 | 3, 12, 25 |
| 17 | CLDN7 | claudin 7 | 181885 | 170291 | 181856 | 16, 13, 27 |
| 17 | CPSF4L | cleavage and polyadenylation specific factor 4 like | 187959 | 141219 | 179604 | |
| 17 | CLUH | clustered mitochondria homolog | 132361 | 007168 | 132359 | 3, 21, 1 |
| 17 | CDRT1 | CMT1A duplicated region transcript 1 | 241322 | 175106 | 283295 | |
| 17 | CDRT1 | CMT1A duplicated region transcript 1 | 283295 | 241322 | 251537 | |
| 17 | CDRT15 | CMT1A duplicated region transcript 15 | 223510 | 006695 | 125430 | 38, |
| 17 | CDRT15L2 | CMT1A duplicated region transcript 15-like 2 | 214819 | 170298 | 205212 | 38, |
| 17 | CDRT4 | CMT1A duplicated region transcript 4 | 239704 | 125409 | 259024 | 16, 38, 44 |
| 17 | COPZ2 | coatomer protein complex subunit zeta 2 | 005243 | 108465 | 082641 | 6, 40, 15 |
| 17 | COASY | Coenzyme A synthase | 068120 | 108786 | 108788 | 36, 2, 39 |
| 17 | CCDC103 | coiled-coil domain containing 103 | 167131 | 108883 | 214447 | 42, 43, 38 |
| 17 | CCDC137 | coiled-coil domain containing 137 | 185298 | 204237 | 214087 | 3, 9, 27 |
| 17 | CCDC144NL | coiled-coil domain containing 144 family, N-terminal like | 205212 | 214819 | 124422 | 22, 38, 23 |
| 17 | CCDC144A | coiled-coil domain containing 144A | 170160 | 197566 | 266302 | |
| 17 | CCDC182 | coiled-coil domain containing 182 | 166329 | 153944 | 181610 | |
| 17 | CCDC40 | coiled-coil domain containing 40 | 141519 | 167291 | 171298 | |
| 17 | CCDC42 | coiled-coil domain containing 42 | 161973 | 133026 | 183318 | 38, 10, 33 |
| 17 | CCDC43 | coiled-coil domain containing 43 | 180329 | 180336 | 161692 | 21, 25, 40 |
| 17 | CCDC47 | coiled-coil domain containing 47 | 108588 | 125695 | 198231 | 43, 13, 40 |
| 17 | CCDC57 | coiled-coil domain containing 57 | 176155 | 169710 | 141526 | |
| 17 | COIL | coilin | 121058 | 121060 | 121064 | 11, 9, 38 |
| 17 | COL1A1 | collagen type I alpha 1 chain | 108821 | 108823 | 167105 | 11, 6, 12 |
| 17 | CSF3 | colony stimulating factor 3 | 108342 | 108344 | 008838 | 9, 20, 35 |
| 17 | C1QBP | complement C1q binding protein | 108561 | 129197 | 005100 | 21, 3, 41 |
| 17 | C1QL1 | complement C1q like 1 | 131094 | 186185 | 172992 | 28, 6, 8 |
| 17 | COG1 | component of oligomeric golgi complex 1 | 166685 | 180616 | 133193 | 31, 40, 38 |
| 17 | CNTNAP1 | contactin associated protein 1 | 108797 | 184451 | 108799 | 18, 26, 14 |
| 17 | COPRS | coordinator of PRMT5 and differentiation stimulator | 172301 | 131242 | 108651 | 34, 33, 17 |
| 17 | COPS3 | COP9 signalosome subunit 3 | 141030 | 154803 | 205309 | 31, 25, 15 |
| 17 | CORO6 | coronin 6 | 167549 | 198720 | 141298 | 26, 18, 25 |
| 17 | CRHR1 | corticotropin releasing hormone receptor 1 | 120088 | 263715 | 185294 | 14, 8, 27 |
| 17 | CRHR1 | corticotropin releasing hormone receptor 1 | 263715 | 225190 | 120088 | 14, 8, 27 |
| 17 | COX10 | COX10, heme A: farnesyltransferase cytochrome c oxidase assembly factor | 006695 | 153976 | 223510 | 43, 37, 38 |
| 17 | COX11 | COX11, cytochrome c oxidase copper chaperone | 166260 | 141198 | 166263 | 11, 14, 4 |
| 17 | CRK | CRK proto-oncogene, adaptor protein | 167193 | 108953 | 197879 | 3, 45, 23 |
| 17 | CRYBA1 | crystallin beta A1 | 108255 | 221995 | 108256 | 14, 12, 10 |
| 17 | CTC1 | CST telomere replication complex component 1 | 178971 | 178999 | 178921 | 39, 22, 41 |
| 17 | CTDNEP1 | CTD nuclear envelope phosphatase 1 | 175826 | 262526 | 262302 | 43, 27, 9 |
| 17 | CLEC10A | C-type lectin domain family 10 member A | 132514 | 174326 | 161944 | 1, 5, 6 |
| 17 | CUEDC1 | CUE domain containing 1 | 180891 | 181610 | 136451 | 34, 26, 13 |
| 17 | CWC25 | CWC25 spliceosome associated protein homolog | 273559 | 276293 | 275489 | 39, 43, 1 |
| 17 | CXCL16 | C-X-C motif chemokine ligand 16 | 161921 | 161920 | 141497 | 36, 10, 37 |
| 17 | CDK12 | cyclin dependent kinase 12 | 167258 | 125686 | 171532 | |
| 17 | CDK3 | cyclin dependent kinase 3 | 250506 | 261408 | 167880 | 22, 23, 16 |
| 17 | CDK5R1 | cyclin dependent kinase 5 regulatory subunit 1 | 176749 | 108671 | 176658 | 5, 38, |
| 17 | CNTD1 | cyclin N-terminal domain containing 1 | 176563 | 183978 | 126581 | 38, 16, 40 |
| 17 | CTNS | cystinosin, lysosomal cystine transporter | 040531 | 197417 | 213977 | 9, 5, 38 |
| 17 | CYB5D1 | cytochrome b5 domain containing 1 | 182224 | 183011 | 170004 | 42, 40, 26 |
| 17 | CYB5D2 | cytochrome b5 domain containing 2 | 167740 | 074755 | 185722 | 29, 13, 3 |
| 17 | CYB561 | cytochrome b561 | 008283 | 170921 | 159640 | 23, 27, 2 |
| 17 | COA3 | cytochrome c oxidase assembly factor 3 | 183978 | 126562 | 176563 | 29, 15, 34 |
| 17 | CYGB | cytoglobin | 161544 | 129667 | 214140 | 26, 15, 31 |
| 17 | CYTH1 | cytohesin 1 | 108669 | 187775 | 055483 | 8, 39, 34 |
| 17 | CRLF3 | cytokine receptor like factor 3 | 176390 | 266733 | 176208 | |
| 17 | DBF4B | DBF4 zinc finger B | 161692 | 180329 | 073670 | 39, 5, 22 |
| 17 | DCAF7 | DDB 1 and CUL4 associated factor 7 | 136485 | 173826 | 136463 | 22, 1, 14 |
| 17 | DDX42 | DEAD-box helicase 42 | 198231 | 108588 | 108592 | |
| 17 | DDX5 | DEAD-box helicase 5 | 108654 | 256525 | 258890 | 42, 39, 36 |
| 17 | DHX33 | DEAH-box helicase 33 | 005100 | 108561 | 072849 | 44, 23, 3 |
| 17 | DHX40 | DEAH-box helicase 40 | 108406 | 175155 | 141367 | 29, 45, 11 |
| 17 | DHX8 | DEAH-box helicase 8 | 067596 | 175906 | 175832 | 24, 42, 43 |
| 17 | DHRS11 | dehydrogenase/reductase 11 | 278535 | 278311 | 278619 | 3, 16, 40 |
| 17 | DHRS13 | dehydrogenase/reductase 13 | 167536 | 132589 | 109118 | 40, 9, 5 |
| 17 | DHRS7B | dehydrogenase/reductase 7B | 109016 | 124422 | 178307 | 6, |
| 17 | DHRS7C | dehydrogenase/reductase 7C | 184544 | 154914 | 214978 | 15, 25, |
| 17 | DCAKD | dephospho-CoA kinase domain containing | 172992 | 131094 | 136448 | 9, 27, 28 |
| 17 | DERL2 | derlin 2 | 072849 | 005100 | 167842 | 30, 2, 20 |
| 17 | DRG2 | develppmentally regulated GTP binding protein 2 | 108591 | 141034 | 091536 | 9, 2, 39 |
| 17 | DDX52 | DExD-bex helicase 52 | 278053 | 275066 | 275410 | 4, 30, 5 |
| 17 | DHX58 | DExH-box helicase 58 | 108771 | 267221 | 108773 | 39, 4, 36 |
| 17 | DGKE | diacylglycerol kinase epsilcn | 153933 | 214226 | 121060 | 9, 22, 1 |
| 17 | DCXR | dicarbcnyl and L-xylulcse reductase | 169738 | 169750 | 169733 | 33, 34, 19 |
| 17 | DUS1L | dihydrouridine synthase 1 like | 169718 | 169727 | 169710 | 3, 9, 40 |
| 17 | DPH1 | diphthamide biosynthesis 1 | 108963 | 185924 | 262664 | 26, 2, 22 |
| 17 | DLG4 | discs large MAGUK scaffold protein 4 | 132535 | 141505 | 072778 | |
| 17 | DVL2 | dishevelled segment polarity protein 2 | 004975 | 072778 | 040633 | 24, 9, 39 |
| 17 | DLX3 | distal-less homeobox 3 | 064195 | 108813 | 005884 | 10, 32, 13 |
| 17 | DLX4 | distal-less homeobox 4 | 108813 | 176358 | 064195 | 32, 6, 35 |
| 17 | LIG3 | DNA ligase 3 | 005156 | 198783 | 092871 | 43, 9, 7 |
| 17 | POLDIP2 | DNA polymerase delta interacting protein 2 | 004142 | 109079 | 244045 | 18, 34, 2 |
| 17 | POLG2 | DNA polymerase gamma 2, accessory subunit | 256525 | 271605 | 108654 | 9, 1, 25 |
| 17 | DNAJC7 | DnaJ heat shock protein family (Hsp40) member C7 | 168259 | 173786 | 168256 | |
| 17 | DOC2B | double C2 domain beta | 272636 | 268320 | 181031 | 27, 17, 14 |
| 17 | DUSP14 | dual specificity phosphatase 14 | 276023 | 276234 | 275066 | 9, 6, 31 |
| 17 | DUSP3 | dual specificity phcsphatase 3 | 108861 | 167941 | 231256 | 35, 24, 9 |
| 17 | DNAH17 | dynein axonemal heavy chain 17 | 187775 | 087157 | 108669 | 38, 18, 8 |
| 17 | DNAH2 | dynein axonemal heavy chain 2 | 183914 | 108947 | 132510 | 26, 23, 42 |
| 17 | DNAH9 | dynein axonemal heavy chain 9 | 007174 | 188803 | 154957 | 42, 38, 20 |
| 17 | DNAI2 | dynein axonemal intermediate chain 2 | 171595 | 141540 | 196169 | 38, 20, 44 |
| 17 | DYNLL2 | dynein light chain LC8-type 2 | 264364 | 136450 | 141194 | 38, 15, 40 |
| 17 | DRC3 | dynein regulatory complex subunit 3 | 171962 | 175662 | 171953 | |
| 17 | EVI2A | ecotropic viral integration site 2A | 126860 | 265118 | 131242 | 22, 6, 8 |
| 17 | EVI2B | ecotropic viral integration site 2B | 185862 | 126861 | 265118 | 5, 36, 7 |
| 17 | ENPP7 | ectonucleotide pyrophosphatase/phosphodiesterase 7 | 182156 | 167281 | 173894 | 19, 17, 37 |
| 17 | EFCAB13 | EF-hand calcium binding domain 13 | 178852 | 259753 | 141279 | 45, 4, 25 |
| 17 | EFCAB3 | EF-hand calcium binding domain 3 | 172421 | 270033 | 087995 | 38, |
| 17 | EFCAB5 | EF-hand calcium binding domain 5 | 176927 | 141298 | 126653 | 38, 22, 10 |
| 17 | ELAC2 | elaC ribonuclease Z 2 | 006744 | 006740 | 153976 | 3, 24, 39 |
| 17 | EFTUD2 | elongation factor Tu GTP binding domain containing 2 | 108883 | 131097 | 167131 | 43, 24, 21 |
| 17 | ELP5 | elongator acetyltransferase complex subunit 5 | 170291 | 262302 | 181885 | 9, 14, 3 |
| 17 | ENGASE | endo-beta-N-acetylglucosaminidase | 167280 | 173918 | 167281 | 18, 7, 1 |
| 17 | ENDOV | endonuclease V | 173818 | 173821 | 171246 | 20, 39, 44 |
| 17 | ERN1 | endoplasmic reticulum to nucleus signaling 1 | 178607 | 108622 | 136478 | 45, 25, 7 |
| 17 | EZH1 | enhancer of zeste 1 polycomb repressive complex 2 subunit | 108799 | 108797 | 131477 | 29, 39, 26 |
| 17 | ENO3 | enolase 3 | 108515 | 108518 | 091640 | 25, 15, 18 |
| 17 | EVPL | envoplakin | 167880 | 250506 | 167881 | 40, 4, 30 |
| 17 | EVPLL | envoplakin like | 214860 | 176974 | 171916 | 9, 38, 35 |
| 17 | EPX | eosinophil peroxidase | 121053 | 255713 | 011143 | 7, 39, 5 |
| 17 | EFNB3 | ephrin B3 | 108947 | 141499 | 183914 | 27, 12, 8 |
| 17 | EPN2 | epsin 2 | 072134 | 189152 | 108641 | 24, 8, 26 |
| 17 | EPN3 | epsin 3 | 049283 | 136449 | 006282 | |
| 17 | EMC6 | ER membrane protein complex subunit 6 | 127774 | 257950 | 083454 | 29, 6, 35 |
| 17 | ERAL1 | Era like 12S mitochondrial rRNA chaperone 1 | 132591 | 173065 | 132589 | 19, 13, 27 |
| 17 | ERBB2 | erb-b2 receptor tyrosine kinase 2 | 141736 | 161395 | 141741 | 23, 18, 16 |
| 17 | EME1 | essential meiotic structure-specific endonuclease 1 | 154920 | 108826 | 108829 | 18, 34, 13 |
| 17 | ETV4 | ETS variant 4 | 175832 | 067596 | 005102 | 37, 27, 16 |
| 17 | EIF1 | eukaryotic translation initiation factor 1 | 173812 | 128422 | 184502 | 1, 11, 43 |
| 17 | EIF4A1 | eukaryotic translation initiation factor 4A1 | 161960 | 277957 | 129226 | 9, 21, 3 |
| 17 | EIF4A3 | eukaryotic translation initiation factor 4A3 | 141543 | 171298 | 141527 | |
| 17 | EIF5A | eukaryotic translation initiation factor 5A | 132507 | 006047 | 132522 | 12, 21, 23 |
| 17 | EXOC7 | exocyst complex component 7 | 182473 | 182687 | 129654 | 34, 4, 40 |
| 17 | FAM20A | FAM20A, golgi associated secretory pathway pseudokinase | 108950 | 108946 | 141338 | 18, 38, 17 |
| 17 | FAM104A | family with sequence similarity 104 member A | 133193 | 166685 | 141219 | 38, 24, 41 |
| 17 | FAM106A | Family with sequence similarity 106 member A | 213077 | 171916 | 189375 | 25, 15, 12 |
| 17 | FAM117A | family with sequence similarity 117 member A | 121104 | 121073 | 136504 | 29, 38, 36 |
| 17 | FAM134C | family with sequence similarity 134 member C | 141699 | 131470 | 131462 | 42, 43, 29 |
| 17 | FAM171A2 | family with sequence similarity 171 member A2 | 161682 | 030582 | 005961 | 13, 12, 8 |
| 17 | FAM187A | family with sequence similarity 187 member A | 214447 | 167131 | 131095 | 42, 43, 38 |
| 17 | FAM222B | family with sequence similarity 222 member B | 173065 | 076604 | 132591 | 35, 38, 13 |
| 17 | FAM57A | family with sequence similarity 57 member A | 167695 | 141252 | 179409 | |
| 17 | FAM64A | family with sequence similarity 64 member A | 129195 | 129221 | 091622 | 3, 34, 12 |
| 17 | FAM83G | family with sequence similarity 83 member G | 188522 | 154025 | 154016 | 9, 12, 3 |
| 17 | FAAP100 | Fanconi anemia core complex associated protein 100 | 185504 | 186765 | 182446 | 29, 22, 27 |
| 17 | FBF1 | Fas binding factor 1 | 188878 | 267426 | 161533 | 25, 26, 40 |
| 17 | FSCN2 | fascin actin-bundling protein 2, retinal | 186765 | 184009 | 185504 | 14, 22, 27 |
| 17 | FADS6 | fatty acid desaturase 6 | 172782 | 161513 | 182040 | 2, 8, 35 |
| 17 | FASN | fatty acid synthase | 169710 | 169718 | 176155 | 33, 35, 1 |
| 17 | FBXL20 | F-box and leucine rich repeat protein 20 | 108306 | 141750 | 125686 | 23, 24, 18 |
| 17 | FBXW10 | F-box and WD repeat domain containing 10 | 171931 | 108448 | 171928 | 38, 32, 8 |
| 17 | FBXO39 | F-box protein 39 | 177294 | 132530 | 167858 | 38, 8, |
| 17 | FBXO47 | F-box protein 47 | 204952 | 263874 | 161381 | 38, |
| 17 | FDXR | ferredoxin reductase | 161513 | 161509 | 172782 | 2, 36, 38 |
| 17 | FGF11 | fibroblast growth factor 11 | 161958 | 181284 | 170175 | 2, 14, 23 |
| 17 | FNDC8 | fibronectin type III domain containing 8 | 073598 | 185379 | 073536 | 38, 17, 8 |
| 17 | FKBP10 | FK506 binding protein 10 | 141756 | 141696 | 141698 | 45, 10, 35 |
| 17 | FLII | FLII, actin remodeling protein | 177731 | 131899 | 177427 | 9, 42, 22 |
| 17 | FLOT2 | flotillin 2 | 132589 | 132591 | 167536 | 18, 9, 21 |
| 17 | FXR2 | FMR1 autosomal homolog 2 | 129245 | 129194 | 129214 | 34, 26, 21 |
| 17 | FLCN | folliculin | 154803 | 264187 | 141030 | 25, 1, 26 |
| 17 | FOXJ1 | forkhead box J1 | 129654 | 182473 | 141576 | 27, 20, 44 |
| 17 | FOXK2 | forkhead box K2 | 141568 | 141562 | 141580 | |
| 17 | FOXN1 | forkhead box N1 | 109101 | 007216 | 109103 | 24, 18, 39 |
| 17 | FMNL1 | formin like 1 | 184922 | 168517 | 184361 | 5, 21, 41 |
| 17 | FZD2 | frizzled class receptor 2 | 180340 | 186566 | 180336 | 29, 12, 19 |
| 17 | FN3K | fructosamine 3 kinase | 167363 | 280575 | 141556 | 40, 26, 31 |
| 17 | FN3KRP | fructosamine 3 kinase related protein | 141560 | 141542 | 280575 | 9, 17, 3 |
| 17 | FTSJ3 | FtsJ homolog 3 | 108592 | 198231 | 087191 | 24, 39, 43 |
| 17 | GPS1 | G protein pathway suppressor 1 | 169727 | 169733 | 169718 | 1, 2, 31 |
| 17 | GPS2 | G protein pathway suppressor 2 | 132522 | 132507 | 261915 | 21, 35, 40 |
| 17 | GNA13 | G protein subunit alpha 13 | 120063 | 176809 | 108370 | 22, 39, 26 |
| 17 | GNGT2 | G protein subunit gamma transducin 2 | 167083 | 167080 | 108798 | 14, 22, 39 |
| 17 | GPR142 | G protein-coupled receptor 142 | 257008 | 204347 | 170412 | |
| 17 | GPR179 | G protein-coupled receptor 179 | 277399 | 278845 | 274211 | 15, 14, 8 |
| 17 | GPRC5C | G protein-coupled receptor class C group 5 member C | 170412 | 257008 | 167851 | 3, 37, 15 |
| 17 | GABARAP | GABA type A receptor-associated protein | 170296 | 040633 | 262526 | 40, 1, 33 |
| 17 | GALK1 | galactokinase 1 | 108479 | 132470 | 132475 | 1, 13, 29 |
| 17 | GALR2 | galanin receptor 2 | 182687 | 186919 | 182473 | 6, |
| 17 | LGALS3BP | galectin 3 binding protein | 108679 | 178404 | 171302 | 7, 13, 27 |
| 17 | LGALS9 | galectin 9 | 168961 | 266728 | 007171 | 36, 27, 5 |
| 17 | LGALS9B | galectin 9B | 170298 | 128487 | 214819 | 13, 37, 5 |
| 17 | LGALS9C | galectin 9C | 171916 | 214860 | 213077 | 16, 13, 5 |
| 17 | GGNBP2 | gametogenetin binding protein 2 | 278311 | 277161 | 278535 | 4, 11, 26 |
| 17 | GGT6 | gamma-glutamyltransferase 6 | 167741 | 132382 | 188176 | 13, 17, 42 |
| 17 | GJD3 | gap junction protein delta 3 | 183153 | 131759 | 131747 | 13, 20, 45 |
| 17 | GJC1 | gap junction protein gamma 1 | 182963 | 073670 | 131097 | 12, 32, 44 |
| 17 | GSDMA | gasdermin A | 167914 | 204913 | 108344 | 24, 35, 18 |
| 17 | GSDMB | gasdermin B | 073605 | 186075 | 172057 | 40, 16, 39 |
| 17 | GIP | gastric inhibitory polypeptide | 159224 | 159210 | 159217 | 37, 16, |
| 17 | GAST | gastrin | 184502 | 173812 | 173805 | 37, |
| 17 | GEMIN4 | gem nuclear organelle associated protein 4 | 179409 | 167695 | 167699 | 38, 21, 9 |
| 17 | GSG2 | germ cell associated 2, haspin | 177602 | 083457 | 074356 | 22, 38, 21 |
| 17 | GHDC | GH3 domain containing | 167925 | 161610 | 173757 | 36, 2, 26 |
| 17 | GID4 | GID complex subunit 4 homolog | 141034 | 171953 | 108591 | 4, 14, 38 |
| 17 | GIT1 | GIT ArfGAP 1 | 108262 | 167543 | 198720 | 25, 43, 27 |
| 17 | GFAP | glial fibrillary acidic protein | 131095 | 214447 | 186185 | 8, 26, 18 |
| 17 | GLP2R | glucagon like peptide 2 receptor | 065325 | 214978 | 109047 | 26, 9, 20 |
| 17 | GCGR | glucagon receptor | 215644 | 183048 | 225663 | 19, 17, 28 |
| 17 | G6PC | glucose-6-phosphatase catalytic subunit | 131482 | 131471 | 266967 | |
| 17 | G6PC3 | glucose-6-phosphatase catalytic subunit 3 | 141349 | 161654 | 108840 | 27, 43, 35 |
| 17 | GAA | glucosidase alpha, acid | 171298 | 141519 | 141543 | 38, 32, 36 |
| 17 | GRIN2C | glutamate ionotropic receptor NMDA type subunit 2C | 161509 | 109066 | 161513 | 14, 40, 38 |
| 17 | QRICH2 | glutamine rich 2 | 129646 | 185262 | 161542 | 40, 42, 32 |
| 17 | GDPD1 | glycerophosphodiester phosphodiesterase domain containing 1 | 153982 | 167447 | 175155 | 37, 45, 28 |
| 17 | GLTPD2 | glycolipid transfer protein domain containing 2 | 182327 | 182853 | 142507 | 17, 4, 28 |
| 17 | GP1BA | glycoprotein Ib platelet alpha subunit | 185245 | 205710 | 108528 | 22, 5, 12 |
| 17 | GLOD4 | glyoxalase domain containing 4 | 167699 | 179409 | 281182 | 30, 4, 9 |
| 17 | GGA3 | golgi associated, gamma adaptin ear containing, ARF binding protein 3 | 125447 | 125450 | 125445 | 24, 42, 43 |
| 17 | GOSR1 | golgi SNAP receptor complex member 1 | 108587 | 108582 | 266733 | 9, 28, 30 |
| 17 | GOSR2 | golgi SNAP receptor complex member 2 | 108433 | 158955 | 262633 | 7, 31, 29 |
| 17 | GPATCH8 | G-patch domain containing 8 | 186566 | 005961 | 180340 | 15, 21, 30 |
| 17 | GRN | granulin precursor | 030582 | 013306 | 161682 | |
| 17 | GRAPL | GRB2 related adaptor protein like | 189152 | 154016 | 072134 | |
| 17 | GRAP | GRB2-related adaptor protein | 154016 | 188522 | 189152 | 21, 41, 5 |
| 17 | GAS2L2 | growth arrest specific 2 like 2 | 270765 | 270885 | 271447 | 27, 20, |
| 17 | GAS7 | growth arrest specific 7 | 007237 | 109047 | 006788 | 8, 1, 35 |
| 17 | GRB2 | growth factor receptor bound protein 2 | 177885 | 125454 | 177728 | 22, 36, 41 |
| 17 | GRB7 | growth factor receptor bound protein 7 | 141738 | 141741 | 161405 | 13, 9, 24 |
| 17 | GH1 | growth hormone 1 | 259384 | 204414 | 007312 | 31, 11, 8 |
| 17 | GH2 | growth hormone 2 | 136487 | 213218 | 136488 | 32, 31, 34 |
| 17 | GSG1L2 | GSG1 like 2 | 214978 | 184544 | 065325 | |
| 17 | GUCY2D | guanylate cyclase 2D, retinal | 132518 | 170037 | 179593 | 40, 14, 33 |
| 17 | H3F3B | H3 histone family member 3B | 132475 | 108479 | 132478 | |
| 17 | HEATR6 | HEAT repeat containing 6 | 068097 | 189050 | 167434 | 31, 24, 2 |
| 17 | HEATR9 | HEAT repeat containing 9 | 270379 | 270806 | 271503 | 38, 8, |
| 17 | HSPB9 | heat shock protein family B (small) member 9 | 260325 | 267261 | 108774 | 38, 28, 20 |
| 17 | HSF5 | heat shock transcription factor 5 | 176160 | 108375 | 108389 | 38, 39, 8 |
| 17 | HELZ | helicase with zinc finger | 198265 | 108878 | 197170 | 22, 13, 1 |
| 17 | HN1 | hematological and neurological expressed 1 | 189159 | 125458 | 188612 | 34, 43, 3 |
| 17 | HS3ST3A1 | heparan sulfate-glucosamine 3-sulfotransferase 3A1 | 153976 | 006744 | 006695 | 32, 2, 25 |
| 17 | HS3ST3B1 | heparan sulfate-glucosamine 3-sulfotransferase 3B1 | 125430 | 223510 | 109099 | 32, 39, 24 |
| 17 | HGS | hepatocyte growth factor-regulated tyrosine kinase substrate | 185359 | 214087 | 262814 | |
| 17 | HES7 | hes family bHLH transcription factor 7 | 179111 | 179148 | 179094 | 6, 9, 27 |
| 17 | HEXIM1 | hexamethylene bisacetamide inducible 1 | 186834 | 181513 | 168517 | 4, 28, 32 |
| 17 | HEXIM2 | hexamethylene bisacetamide inducible 2 | 168517 | 186834 | 184922 | 29, 14, 27 |
| 17 | HEXDC | hexosaminidase D | 169660 | 181396 | 178927 | 27, 7, 17 |
| 17 | HIC1 | HIC ZBTB transcriptional repressor 1 | 177374 | 262664 | 070366 | |
| 17 | HID1 | HID1 domain containing | 167861 | 182938 | 109089 | 42, 26, 33 |
| 17 | HIGD1B | HIG1 hypoxia inducible domain family member 1B | 131097 | 182963 | 108883 | 15, 28, 2 |
| 17 | HDAC5 | histone deacetylase 5 | 108840 | 141349 | 125319 | 1, 34, 27 |
| 17 | HLF | HLF, PAR bZIP transcription factor | 108924 | 166263 | 108960 | 19, 13, 8 |
| 17 | HNF1B | HNF1 homeobox B | 275410 | 278053 | 273513 | 17, 3, 29 |
| 17 | HOXB1 | homeobox B1 | 120094 | 141293 | 173917 | 12, 38, |
| 17 | HOXB13 | homeobox B13 | 159184 | 229637 | 170703 | 33, 37, 16 |
| 17 | HOXB2 | homeobox B2 | 173917 | 120094 | 120093 | |
| 17 | HOXB3 | homeobox B3 | 120093 | 173917 | 182742 | 38, 12, 44 |
| 17 | HOXB4 | homeobox B4 | 182742 | 120093 | 120075 | 44, 15, 28 |
| 17 | HOXB5 | homeobox B5 | 120075 | 182742 | 108511 | 16, 33, 20 |
| 17 | HOXB6 | homeobox B6 | 108511 | 120075 | 260027 | 16, 1, 26 |
| 17 | HOXB7 | homeobox B7 | 260027 | 108511 | 120068 | 16, 25, 9 |
| 17 | HOXB8 | homeobox B8 | 120068 | 260027 | 170689 | 16, 17, 33 |
| 17 | HOXB9 | homeobox B9 | 170689 | 120068 | 159182 | 16, 38, 17 |
| 17 | HAP1 | huntingtin associated protein 1 | 173805 | 184502 | 173801 | |
| 17 | HSD17B1 | hydroxysteroid 17-beta dehydrogenase 1 | 108786 | 108784 | 068120 | 32, 23, 4 |
| 17 | HCRT | hypocretin neuropeptide precursor | 161610 | 089558 | 167925 | 10, 8, |
| 17 | IKZF3 | IKAROS family zinc finger 3 | 161405 | 141738 | 186075 | 22, 41, 30 |
| 17 | INCA1 | inhibitor of CDK, cyclin A1 interacting protein 1 | 196388 | 108509 | 129250 | 4, 38, 27 |
| 17 | INPP5K | inositol polyphosphate-5-phosphatase K | 132376 | 197879 | 174238 | 31, 22, 14 |
| 17 | IGF2BP1 | insulin like growth factor 2 mRNA binding protein 1 | 159217 | 159224 | 167080 | 12, 38, 14 |
| 17 | IGFBP4 | insulin like growth factor binding protein 4 | 141753 | 131747 | 131746 | 37, 23, 20 |
| 17 | INTS2 | integrator complex subunit 2 | 108506 | 136492 | 108510 | 39, 34, 13 |
| 17 | ITGA2B | integrin subunit alpha 2b | 005961 | 161682 | 186566 | 7, 39, 36 |
| 17 | ITGA3 | integrin subunit alpha 3 | 005884 | 064195 | 005882 | 17, 4, 44 |
| 17 | ITGAE | integrin subunit alpha E | 083457 | 083454 | 177602 | 33, 17, 7 |
| 17 | ITGB3 | integrin subunit beta 3 | 259207 | 198336 | 259753 | 17, 40, 1 |
| 17 | ITGB4 | integrin subunit beta 4 | 132470 | 161526 | 108479 | 23, 27, 18 |
| 17 | ICAM2 | intercellular adhesion molecule 2 | 108622 | 224383 | 178607 | 43, 29, 36 |
| 17 | IFI35 | interferon induced protein 35 | 068079 | 131469 | 108828 | 3, 34, 13 |
| 17 | IFT20 | intraflagellar transport 20 | 109083 | 109084 | 109079 | 1, 36, 26 |
| 17 | JUP | junction plakoglobin | 173801 | 173805 | 141696 | 18, 28, 4 |
| 17 | KPNA2 | karyopherin subunit alpha 2 | 182481 | 186665 | 196704 | 3, 38, 37 |
| 17 | KPNB1 | karyopherin subunit beta 1 | 108424 | 141279 | 198933 | 25, 21, 24 |
| 17 | KANSL1 | KAT8 regulatory NSL complex subunit 1 | 120071 | 256762 | 228696 | 11, 37, 5 |
| 17 | KLHL10 | kelch like family member 10 | 161594 | 141698 | 178502 | |
| 17 | KLHL11 | kelch like family member 11 | 178502 | 161594 | 131473 | 38, 25, 8 |
| 17 | KRT10 | keratin 10 | 186395 | 173908 | 167920 | 35, 19, 9 |
| 17 | KRT12 | keratin 12 | 187242 | 167920 | 171431 | 14, 8, |
| 17 | KRT13 | keratin 13 | 171401 | 126337 | 171346 | 13, 24, 30 |
| 17 | KRT14 | keratin 14 | 186847 | 171403 | 186832 | 30, 18, 4 |
| 17 | KRT15 | keratin 15 | 171346 | 171401 | 171345 | 13, 18, 24 |
| 17 | KRT16 | keratin 16 | 186832 | 186847 | 128422 | 13, 18, 35 |
| 17 | KRT17 | keratin 17 | 128422 | 186832 | 173812 | 18, 9, 13 |
| 17 | KRT19 | keratin 19 | 171345 | 171346 | 171403 | 28, 23, 16 |
| 17 | KRT20 | keratin 20 | 171431 | 187242 | 108244 | 16, 37, 25 |
| 17 | KRT222 | keratin 222 | 213424 | 264058 | 167916 | 31, 2, 36 |
| 17 | KRT23 | keratin 23 | 108244 | 171431 | 196859 | 32, 18, 23 |
| 17 | KRT24 | keratin 24 | 167916 | 213424 | 204897 | 30, 14, 32 |
| 17 | KRT25 | keratin 25 | 204897 | 167916 | 186393 | 15, |
| 17 | KRT26 | keratin 26 | 186393 | 204897 | 171446 | |
| 17 | KRT27 | keratin 27 | 171446 | 186393 | 173908 | 15, 18, 35 |
| 17 | KRT28 | keratin 28 | 173908 | 171446 | 186395 | 10, 15, 35 |
| 17 | KRT31 | keratin 31 | 094796 | 131737 | 108417 | 13, 18, 24 |
| 17 | KRT32 | keratin 32 | 108759 | 171360 | 197079 | 16, 20, 30 |
| 17 | KRT33A | keratin 33A | 006059 | 186860 | 131738 | 38, |
| 17 | KRT33B | keratin 33B | 131738 | 006059 | 131737 | 17, 15, |
| 17 | KRT34 | keratin 34 | 131737 | 131738 | 094796 | 35, 7, 19 |
| 17 | KRT35 | keratin 35 | 197079 | 108759 | 126337 | 18, 16, 10 |
| 17 | KRT36 | keratin 36 | 126337 | 197079 | 171401 | 16, 20, |
| 17 | KRT37 | keratin 37 | 108417 | 094796 | 171360 | 16, |
| 17 | KRT38 | keratin 38 | 171360 | 108417 | 108759 | 16, |
| 17 | KRT39 | keratin 39 | 196859 | 108244 | 204889 | 32, |
| 17 | KRT40 | keratin 40 | 204889 | 196859 | 212899 | 31, 42, 15 |
| 17 | KRT9 | keratin 9 | 171403 | 171345 | 186847 | |
| 17 | KRTAP1-1 | keratin associated protein 1-1 | 188581 | 221880 | 212725 | |
| 17 | KRTAP1-3 | keratin associated protein 1-3 | 221880 | 204887 | 188581 | |
| 17 | KRTAP1-4 | keratin associated protein 1-4 | 204887 | 221852 | 221880 | |
| 17 | KRTAP1-5 | keratin associated protein 1-5 | 221852 | 212901 | 204887 | 10, 6, |
| 17 | KRTAP16-1 | keratin associated protein 16-1 | 212657 | 212658 | 186860 | 23, |
| 17 | KRTAP17-1 | keratin associated protein 17-1 | 186860 | 212657 | 006059 | 32, |
| 17 | KRTAP2-1 | keratin associated protein 2-1 | 212725 | 188581 | 214518 | |
| 17 | KRTAP2-2 | keratin associated protein 2-2 | 214518 | 212725 | 212724 | 3, |
| 17 | KRTAP2-3 | keratin associated protein 2-3 | 212724 | 214518 | 213417 | 19, 23, 22 |
| 17 | KRTAP2-4 | keratin associated protein 2-4 | 213417 | 212724 | 240871 | |
| 17 | KRTAP29-1 | keratin associated protein 29-1 | 212658 | 180386 | 212657 | |
| 17 | KRTAP3-1 | keratin associated protein 3-1 | 212901 | 212900 | 221852 | |
| 17 | KRTAP3-2 | keratin associated protein 3-2 | 212900 | 212899 | 212901 | 3, 44, 14 |
| 17 | KRTAP3-3 | keratin associated protein 3-3 | 212899 | 204889 | 212900 | |
| 17 | KRTAP4-1 | keratin associated protein 4-1 | 198443 | 244537 | 240542 | 28, |
| 17 | KRTAP4-11 | keratin associated protein 4-11 | 212721 | 212722 | 213416 | |
| 17 | KRTAP4-12 | keratin associated protein 4-12 | 213416 | 212721 | 198090 | |
| 17 | KRTAP4-16 | Keratin associated protein 4-16 | 241241 | 204880 | 212722 | |
| 17 | KRTAP4-2 | keratin associated protein 4-2 | 244537 | 196156 | 198443 | |
| 17 | KRTAP4-3 | keratin associated protein 4-3 | 196156 | 171396 | 244537 | 35, |
| 17 | KRTAP4-4 | keratin associated protein 4-4 | 171396 | 198271 | 196156 | |
| 17 | KRTAP4-5 | keratin associated protein 4-5 | 198271 | 198090 | 171396 | 25, |
| 17 | KRTAP4-6 | keratin associated protein 4-6 | 198090 | 213416 | 198271 | |
| 17 | KRTAP4-7 | keratin associated protein 4-7 | 240871 | 213417 | 204880 | |
| 17 | KRTAP4-8 | keratin associated protein 4-8 | 204880 | 240871 | 241241 | |
| 17 | KRTAP4-9 | keratin associated protein 4-9 | 212722 | 241241 | 212721 | |
| 17 | KRTAP9-1 | keratin associated protein 9-1 | 240542 | 198443 | 239886 | |
| 17 | KRTAP9-2 | keratin associated protein 9-2 | 239886 | 240542 | 204873 | 15, |
| 17 | KRTAP9-3 | keratin associated protein 9-3 | 204873 | 239886 | 187272 | |
| 17 | KRTAP9-4 | keratin associated protein 9-4 | 241595 | 187272 | 198083 | |
| 17 | KRTAP9-6 | keratin associated protein 9-6 | 212659 | 198083 | 180386 | |
| 17 | KRTAP9-7 | keratin associated protein 9-7 | 180386 | 212659 | 212658 | |
| 17 | KRTAP9-8 | keratin associated protein 9-8 | 187272 | 204873 | 241595 | |
| 17 | KRTAP9-9 | keratin associated protein 9-9 | 198083 | 241595 | 212659 | |
| 17 | KIAA0100 | KIAA0100 | 007202 | 167524 | 132581 | 18, 16, 23 |
| 17 | KIAA0753 | KIAA0753 | 198920 | 091622 | 282936 | 29, 38, 42 |
| 17 | KSR1 | kinase suppressor of ras 1 | 141068 | 109046 | 266728 | 1, 26, 38 |
| 17 | KIF18B | kinesin family member 18B | 186185 | 131095 | 131094 | |
| 17 | KIF19 | kinesin family member 19 | 196169 | 171595 | 204347 | 9, 8, 36 |
| 17 | KIF1C | kinesin family member 1C | 129250 | 196388 | 132517 | 34, 40, 35 |
| 17 | KIF2B | kinesin family member 2B | 141200 | 154975 | 141198 | 38, 8, |
| 17 | KRBA2 | KRAB-A domain containing 2 | 184619 | 184650 | 263809 | 24, 40, 45 |
| 17 | LPO | lactoperoxidase | 167419 | 011143 | 005381 | 42, 34, 25 |
| 17 | LRRC37A2 | leucine rich repeat containing 37 member A2 | 238083 | 176681 | 185829 | 31, 1, 26 |
| 17 | LRRC37A3 | leucine rich repeat containing 37 member A3 | 176809 | 108854 | 120063 | 30, 18, 1 |
| 17 | LRRC37A | leucine rich repeat containing 37A | 176681 | 228696 | 238083 | 18, 37, 12 |
| 17 | LRRC37B | leucine rich repeat containing 37B | 185158 | 178691 | 126858 | |
| 17 | LRRC3C | leucine rich repeat containing 3C | 204913 | 172057 | 167914 | 38, |
| 17 | LRRC45 | leucine rich repeat containing 45 | 169683 | 169689 | 169750 | 41, 9, 5 |
| 17 | LRRC46 | leucine rich repeat containing 46 | 141294 | 159111 | 141295 | 42, 38, 22 |
| 17 | LRRC59 | leucine rich repeat containing 59 | 108829 | 154920 | 167107 | 21, 23, 3 |
| 17 | LRRC75A | leucine rich repeat containing 75A | 181350 | 187688 | 141040 | 31, 30, 9 |
| 17 | LASP1 | LIM and SH3 protein 1 | 002834 | 125691 | 263874 | 39, 42, 43 |
| 17 | LIMD2 | LIM domain containing 2 | 136490 | 198909 | 266173 | 41, 21, 22 |
| 17 | LHX1 | LIM homeobox 1 | 273706 | 278619 | 275700 | 40, 27, 12 |
| 17 | LLGL1 | LLGL1, scribble cell polarity complex component | 131899 | 091542 | 177731 | 22, 3, 7 |
| 17 | LLGL2 | LLGL2, scribble cell polarity complex cpmppnent | 073350 | 182173 | 266714 | 28, 3, 16 |
| 17 | LINC00672 | Long intergenic non-protein coding RNA 672 | 263874 | 002834 | 204952 | 27, 31, 10 |
| 17 | LINC00675 | Long intergenic non-protein coding RNA 675 | 263429 | 187824 | 233670 | 3, 33, 16 |
| 17 | LINC00854 | Long intergenic non-protein coding RNA 854 | 236383 | 184988 | 175906 | 23, 38, 44 |
| 17 | LSM12 | LSM12 homolog | 161654 | 091947 | 141349 | 43, 18, 6 |
| 17 | LUC7L3 | LUC7 like 3 pre-mRNA splicing factor | 108848 | 154945 | 173714 | 39, 11, 13 |
| 17 | LYRM9 | LYR motif containing 9 | 232859 | 266202 | 087095 | 17, 8, 38 |
| 17 | KAT2A | lysine acetyltransferase 2A | 108773 | 108771 | 267261 | 1, 18, 9 |
| 17 | KAT7 | lysine acetyltransferase 7 | 136504 | 121104 | 176358 | 38, 42, 24 |
| 17 | KDM6B | lysine demethylase 6B | 132510 | 183914 | 167874 | 20, 22, 14 |
| 17 | LYZL6 | lysozyme like 6 | 275722 | 278023 | 275152 | 38, 10, 8 |
| 17 | MAFG | MAF bZIP transcription factor G | 197063 | 187531 | 183010 | 30, 9, 26 |
| 17 | MFSD11 | major facilitator superfamily domain containing 11 | 092931 | 161547 | 167889 | |
| 17 | MFSD6L | major facilitator superfamily domain containing 6 like | 185156 | 183318 | 276231 | 38, 20, 28 |
| 17 | MSL1 | male specific lethal 1 homolog | 188895 | 126368 | 108349 | 40, 15, 14 |
| 17 | MRC2 | mannose receptor C type 2 | 011028 | 146872 | 173838 | 6, 34, 12 |
| 17 | MPDU1 | mannose-P-dolichol utilization defect 1 | 129255 | 129226 | 129194 | 28, 35, 17 |
| 17 | MGAT5B | mannosyl (alpha-1,6-)-glycoprotein beta-1,6-N-acetyl-glucosaminyltransferase, | 167889 | 092931 | 129657 | 9, 36, 7 |
| | | isozyme B | | | | |
| 17 | MCRIP1 | MAPK regulated corepressor interacting protein 1 | 225663 | 215644 | 182676 | |
| 17 | MILR1 | mast cell immunoglobulin like receptor 1 | 271605 | 261371 | 256525 | 35, 21, 30 |
| 17 | MMP28 | matrix metallopeptidase 28 | 271447 | 270765 | 270806 | 38, 1, 26 |
| 17 | MXRA7 | matrix remodeling associated 7 | 182534 | 070526 | 070495 | |
| 17 | MNT | MAX network transcriptional repressor | 070444 | 141258 | 127804 | 24, 1, 26 |
| 17 | MBTD1 | mbt domain containing 1 | 011258 | 243678 | 011260 | 14, 22, 31 |
| 17 | MKS1 | Meckel syndrome, type 1 | 011143 | 121053 | 167419 | 3, 9, 30 |
| 17 | MED1 | mediator complex subunit 1 | 125686 | 108306 | 167258 | 23, 45, 29 |
| 17 | MED11 | mediator complex subunit 11 | 161920 | 141480 | 161921 | 1, 6, 4 |
| 17 | MED13 | mediator complex subunit 13 | 108510 | 108506 | 270033 | 23, 34, 11 |
| 17 | MED24 | mediator complex subunit 24 | 008838 | 108342 | 126351 | 24, 42, 39 |
| 17 | MED31 | mediator complex subunit 31 | 108590 | 129235 | 279257 | 30, 43, 26 |
| 17 | MED9 | mediator complex subunit 9 | 141026 | 205309 | 108551 | 36, 25, 9 |
| 17 | MEIOC | meiosis specific with coiled-coil domain | 180336 | 180340 | 180329 | |
| 17 | MARCH10 | membrane associated ring-CH-type finger 10 | 173838 | 011028 | 170921 | |
| 17 | MPP2 | membrane palmitoylated protein 2 | 108852 | 161649 | 108849 | 8, 29, 38 |
| 17 | MPP3 | membrane palmitoylated protein 3 | 161647 | 231256 | 161649 | 2, 20, 8 |
| 17 | MEOX1 | mesenchyme homeobox 1 | 005102 | 175832 | 167941 | 1, 27, 25 |
| 17 | METRNL | meteorin like, glial cell differentiation regulator | 176845 | 175711 | 167693 | 36, 6, 4 |
| 17 | METTL16 | methyltransferase like 16 | 127804 | 070444 | 007168 | |
| 17 | METTL23 | methyltransferase like 23 | 181038 | 070495 | 267168 | 43, 22, 31 |
| 17 | METTL2A | methyltransferase like 2A | 087995 | 172421 | 146872 | 41, 3, 29 |
| 17 | MFAP4 | microfibrillar associated protein 4 | 166482 | 166484 | 128482 | |
| 17 | MAPT | microtubule associated protein tau | 186868 | 185294 | 256762 | 26, 8, 14 |
| 17 | MIF4GD | MIF4G domain containing | 125457 | 125445 | 125454 | 9, 40, 13 |
| 17 | MIEN1 | migration and invasion enhancer 1 | 141741 | 141736 | 141738 | 29, 6, 32 |
| 17 | MIS12 | MIS12, kinetochore complex component | 167842 | 072849 | 091592 | 24, 36, 39 |
| 17 | MINK1 | misshapen like kinase 1 | 141503 | 129219 | 108556 | 26, 14, 41 |
| 17 | MIEF2 | mitochondrial elongation factor 2 | 177427 | 177731 | 177302 | 39, 26, 34 |
| 17 | MRPL10 | mitochondrial ribosomal protein L10 | 159111 | 006025 | 141294 | 43, 9, 5 |
| 17 | MRPL12 | mitochondrial ribosomal protein L12 | 262814 | 185359 | 262660 | 34, 33, 41 |
| 17 | MRPL27 | mitochondrial ribosomal protein L27 | 108826 | 015532 | 154920 | 29, 15, 30 |
| 17 | MRPL38 | mitochondrial ribosomal protein L38 | 204316 | 141569 | 267426 | 28, 25, 43 |
| 17 | MRPL45 | mitochondrial ribosomal protein L45 | 278845 | 274611 | 277399 | 43, 1, 34 |
| 17 | MRPL58 | mitochondrial ribosomal protein L58 | 167862 | 109089 | 180901 | |
| 17 | MRPS23 | mitochondrial ribosomal protein S23 | 181610 | 166329 | 180891 | 29, 1, 15 |
| 17 | MRPS7 | mitochondrial ribosomal protein S7 | 125445 | 125447 | 125457 | 30, 23, 21 |
| 17 | MRM1 | mitochondrial rRNA methyltransferase 1 | 278619 | 278535 | 273706 | 9, 34, 6 |
| 17 | MRM3 | mitochondrial rRNA methyltransferase 3 | 171861 | 281182 | 167693 | |
| 17 | MAPK7 | mitogen-activated protein kinase 7 | 166484 | 108641 | 166482 | 39, 25, 41 |
| 17 | MAP2K3 | mitogen-activated protein kinase kinase 3 | 034152 | 274180 | 184185 | 21, 3, 5 |
| 17 | MAP2K4 | mitogen-activated protein kinase kinase 4 | 065559 | 154957 | 141052 | 41, 8, 3 |
| 17 | MAP2K6 | mitogen-activated protein kinase kinase 6 | 108984 | 154265 | 153822 | 25, 15, 6 |
| 17 | MAP3K14 | mitogen-activated protein kinase kinase kinase 14 | 006062 | 184361 | 159314 | 9, 39, 11 |
| 17 | MAP3K3 | mitogen-activated protein kinase kinase kinase 3 | 198909 | 136463 | 136490 | 21, 35, 6 |
| 17 | MLLT6 | MLLT6, PHD finger domain containing | 275023 | 273604 | 279119 | 29, 26, 22 |
| 17 | MLX | MLX, MAX dimerization protein | 108788 | 068120 | 131470 | 21, 33, 6 |
| 17 | MMD | monocyte to macrophage differentiation associated | 108960 | 108924 | 261873 | 1, 23, 22 |
| 17 | MTRNR2L1 | MT-RNR2-like 1 | 256618 | 283566 | 109046 | |
| 17 | MSI2 | musashi RNA binding protein 2 | 153944 | 121057 | 166329 | 22, 13, 39 |
| 17 | MYBBP1A | MYB binding protein 1a | 132382 | 183018 | 167741 | 21, 36, 12 |
| 17 | MYCBPAP | MYCBP associated protein | 136449 | 136444 | 049283 | 38, 2, 32 |
| 17 | MYADML2 | myeloid associated differentiation marker like 2 | 185105 | 183010 | 185269 | 14, 25, 33 |
| 17 | MPO | myeloperoxidase | 005381 | 167419 | 005379 | 5, 7, 40 |
| 17 | MYOCD | myocardin | 141052 | 065559 | 006740 | 42, 26, 33 |
| 17 | MYH1 | myosin heavy chain 1 | 109061 | 264424 | 125414 | 18, 25, 40 |
| 17 | MYH10 | myosin heavy chain 10 | 133026 | 166579 | 161973 | 43, 1, 12 |
| 17 | MYH13 | myosin heavy chain 13 | 006788 | 007237 | 133020 | 8, |
| 17 | MYH2 | myosin heavy chain 2 | 125414 | 109061 | 109063 | 18, 25, 24 |
| 17 | MYH3 | myosin heavy chain 3 | 109063 | 125414 | 133028 | 25, 15, 26 |
| 17 | MYH4 | myosin heavy chain 4 | 264424 | 133020 | 109061 | 40, |
| 17 | MYH8 | myosin heavy chain 8 | 133020 | 006788 | 264424 | 25, 28, 39 |
| 17 | MYO1C | myosin IC | 197879 | 167193 | 132376 | 43, 9, 1 |
| 17 | MYO1D | myosin ID | 176658 | 176749 | 006042 | |
| 17 | MYL4 | myosin light chain 4 | 198336 | 004897 | 259207 | 15, 41, 20 |
| 17 | MPRIP | myosin phosphatase Rho interacting protein | 133030 | 240505 | 179598 | 27, 3, 25 |
| 17 | MYO19 | myosin XIX | 278259 | 273611 | 277161 | |
| 17 | MYO15A | myosin XVA | 091536 | 108591 | 091542 | 31, 38, 8 |
| 17 | MYO15B | myosin XVB | 266714 | 073350 | 108469 | 36, 26, 22 |
| 17 | MYO18A | myosin XVIIIA | 196535 | 063015 | 221995 | 18, 22, 27 |
| 17 | MTMR4 | myotubularin related protein 4 | 108389 | 176160 | 108387 | 43, 22, 26 |
| 17 | NAA38 | N(alpha)-acetyltransferase 38, NatC auxiliary subunit | 183011 | 167874 | 182224 | 29, 33, 15 |
| 17 | NAGLU | N-acetyl-alpha-glucosaminidase | 108784 | 033627 | 108786 | 2, 37, 20 |
| 17 | NAGS | N-acetylglutamate synthase | 161653 | 131096 | 091947 | 43, 17, 40 |
| 17 | NAT9 | N-acetyltransferase 9 (putative) | 109065 | 109062 | 109066 | 2, 31, 26 |
| 17 | NATD1 | N-acetyltransferase domain containing 1 | 274180 | 178307 | 034152 | 29, 2, 18 |
| 17 | NDUFAF8 | NADH: ubiquinone oxidoreductase complex assembly factor 8 | 224877 | 167302 | 157637 | |
| 17 | NACA2 | nascent polypeptide associated complex alpha subunit 2 | 253506 | 121075 | 136492 | 43, 1, 18 |
| 17 | NBR1 | NBR1, autophagy cargo receptor | 188554 | 012048 | 184988 | 42, 38, 29 |
| 17 | NLK | nemo like kinase | 087095 | 232859 | 109084 | |
| 17 | NGFR | nerve growth factor receptor | 064300 | 281126 | 182575 | 26, 27, 10 |
| 17 | NSF | N-ethylmaleimide sensitive factor, vesicle fusing ATPase | 073969 | 283191 | 108379 | 8, 24, 3 |
| 17 | NTN1 | netrin 1 | 065320 | 141506 | 170310 | 27, 13, 14 |
| 17 | NEURL4 | neuralized E3 ubiquitin protein ligase 4 | 215041 | 261915 | 072818 | 31, 22, 14 |
| 17 | NXPH3 | neurexophilin 3 | 182575 | 064300 | 121067 | 31, 2, 14 |
| 17 | NF1 | neurofibromin 1 | 196712 | 181481 | 126861 | 29, 4, 38 |
| 17 | NLGN2 | neuroligin 2 | 169992 | 205544 | 181323 | 27, 29, 12 |
| 17 | NEUROD2 | neuronal differentiation 2 | 171532 | 167258 | 131771 | 8, |
| 17 | NPTX1 | neuronal pentraxin 1 | 171246 | 173818 | 141564 | 8, 2, 14 |
| 17 | NPB | neuropeptide B | 183979 | 141552 | 185813 | 20, 38, |
| 17 | NKIRAS2 | NFKB inhibitor interacting Ras like 2 | 168256 | 168259 | 187595 | 14, 32, 15 |
| 17 | NEK8 | NIMA related kinase 8 | 160602 | 160606 | 076604 | 41, 21, 9 |
| 17 | NOS2 | nitric oxide synthase 2 | 007171 | 168961 | 266202 | 30, 16, 37 |
| 17 | NLRP1 | NLR family pyrin domain containing 1 | 091592 | 167842 | 179314 | 43, 22, 5 |
| 17 | NME1 | NME/NM23 nucleoside diphosphate kinase 1 | 239672 | 008294 | 011052 | |
| 17 | NME2 | NME/NM23 nucleoside diphosphate kinase 2 | 243678 | 011052 | 011258 | 35, 21, 43 |
| 17 | NME1-NME2 | NME1-NME2 readthrough | 011052 | 239672 | 243678 | |
| 17 | NMT1 | N-myristoyltransferase 1 | 136448 | 172992 | 161714 | |
| 17 | NOG | noggin | 183691 | 153930 | 214226 | 36, 5, 33 |
| 17 | NLE1 | notchless homolog 1 | 073536 | 073598 | 141161 | 43, 3, 34 |
| 17 | NOTUM | NOTUM, palmitoleoyl-protein carboxylesterase | 185269 | 185105 | 169696 | 32, 44, 16 |
| 17 | NPLOC4 | NPL4 homolog, ubiquitin recognition factor | 182446 | 185504 | 182612 | 34, 37, 38 |
| 17 | SGSH | N-sulfoglucosamine sulfohydrolase | 181523 | 141527 | 181045 | 34, 2, 36 |
| 17 | NCBP3 | nuclear cap binding subunit 3 | 074356 | 177602 | 004660 | |
| 17 | NFE2L1 | nuclear factor, erythroid 2 like 1 | 082641 | 005243 | 108468 | |
| 17 | NARF | nuclear prelamin A recognition factor | 141562 | 178927 | 141568 | 39, 38, 5 |
| 17 | NCOR1 | nuclear receptor corepressor 1 | 141027 | 011295 | 108474 | 1, 13, 37 |
| 17 | NR1D1 | nuclear receptor subfamily 1 group D member 1 | 126368 | 126351 | 188895 | 26, 18, 3 |
| 17 | NSRP1 | nuclear speckle splicing regulatory protein 1 | 126653 | 176927 | 108576 | 18, 26, 13 |
| 17 | NOL11 | nucleolar protein 11 | 130935 | 154217 | 171634 | 24, 36, 13 |
| 17 | NUP85 | nucleoporin 85 | 125450 | 188612 | 125447 | 3, 39, 21 |
| 17 | NUP88 | nucleoporin 88 | 108559 | 029725 | 129197 | 29, 18, 42 |
| 17 | NXN | nucleoredoxin | 167693 | 171861 | 177370 | |
| 17 | NDEL1 | nudE neurodevelopment protein 1 like 1 | 166579 | 189051 | 133026 | |
| 17 | NUFIP2 | NUFIP2, FMR1 interacting protein 2 | 108256 | 108255 | 160551 | 29, 26, 22 |
| 17 | OR1A1 | olfactory receptor family 1 subfamily A member 1 | 172146 | 172150 | 221882 | |
| 17 | OR1A2 | olfactory receptor family 1 subfamily A member 2 | 172150 | 183024 | 172146 | |
| 17 | OR1D2 | olfactory receptor family 1 subfamily D member 2 | 184166 | 262628 | 183024 | |
| 17 | OR1D5 | olfactory receptor family 1 subfamily D member 5 | 262628 | 132359 | 184166 | |
| 17 | OR1E1 | olfactory receptor family 1 subfamily E member 1 | 180016 | 180090 | 159961 | 33, |
| 17 | OR1E2 | olfactory receptor family 1 subfamily E member 2 | 127780 | 159961 | 141255 | |
| 17 | OR1G1 | olfactory receptor family 1 subfamily G member 1 | 183024 | 184166 | 172150 | |
| 17 | OR3A1 | olfactory receptor family 3 subfamily A member 1 | 180090 | 221882 | 180016 | |
| 17 | OR3A2 | olfactory receptor family 3 subfamily A member 2 | 221882 | 172146 | 180090 | 38, |
| 17 | OR3A3 | olfactory receptor family 3 subfamily A member 3 | 159961 | 180016 | 127780 | |
| 17 | OR4D1 | olfactory receptor family 4 subfamily D member 1 | 141194 | 264364 | 255713 | |
| 17 | OR4D2 | olfactory receptor family 4 subfamily D member 2 | 255713 | 141194 | 121053 | |
| 17 | OMG | oligodendrocyte myelin glycoprotein | 126861 | 196712 | 185862 | 8, 17, 2 |
| 17 | ORMDL3 | ORMDL sphingolipid bipsynthesis regulater 3 | 172057 | 073605 | 204913 | 1, 23, 21 |
| 17 | OTOP2 | otopetrin 2 | 183034 | 182040 | 281273 | 19, 16, 38 |
| 17 | OTOP3 | otopetrin 3 | 182938 | 281273 | 167861 | 14, |
| 17 | ODF4 | outer dense fiber of sperm tails 4 | 184650 | 198844 | 184619 | 38, 37, |
| 17 | OVCA2 | ovarian tumor suppresser candidate 2 | 262664 | 108963 | 177374 | |
| 17 | OXLD1 | oxidoreductase like domain containing 1 | 204237 | 185527 | 185298 | 26, 27, 44 |
| 17 | OSBPL7 | oxysterol binding protein like 7 | 006025 | 073861 | 159111 | 3, 31, 33 |
| 17 | P2RX5-TAX1BP3 | P2RX5-TAX1BP3 readthrough (NMD candidate) | 257950 | 213977 | 127774 | 9, 11, 6 |
| 17 | PPY | pancreatic polypeptide | 108849 | 108852 | 131096 | |
| 17 | PYY | peptide YY | 131096 | 108849 | 161653 | 16, 17, 38 |
| 17 | PTRH2 | peptidyl-tRNA hydrolase 2 | 141378 | 141367 | 062716 | 43, 26, 3 |
| 17 | PER1 | period circadian deck 1 | 179094 | 179111 | 263620 | 26, 42, 39 |
| 17 | PMP22 | peripheral myelin protein 22 | 109099 | 125430 | 125409 | 11, 26, 15 |
| 17 | PEX12 | peroxisemal biogenesis factor 12 | 108733 | 236320 | 006125 | 45, 1, 24 |
| 17 | PHF12 | PHD finger protein 12 | 109118 | 167536 | 179761 | 18, 33, 22 |
| 17 | PHF23 | PHD finger protein 23 | 040633 | 004975 | 170296 | 35, 6, 40 |
| 17 | PNMT | phenylethanolamine N-methyltransferase | 141744 | 173991 | 161395 | 2, 14, 40 |
| 17 | PCYT2 | phosphate cytidylyltransferase 2, ethanolamine | 185813 | 183979 | 187531 | 38, 25, 19 |
| 17 | PCTP | phosphatidylcholine transfer protein | 141179 | 166292 | 153930 | 29, 4, 17 |
| 17 | PEMT | phosphatidylethanolamine N-methyltransferase | 133027 | 108551 | 108557 | 33, 10, 27 |
| 17 | PGS1 | phosphatidylglycerophosphate synthase 1 | 087157 | 184557 | 187775 | 31, 34, 36 |
| 17 | PIGL | phosphatidylinositol glycan anchor biosynthesis class L | 108474 | 141027 | 166582 | 39, 36, 13 |
| 17 | PIGS | phosphatidylinositol glycan anchor biosynthesis class S | 087111 | 109103 | 109107 | 24, 13, 3 |
| 17 | PIGW | phosphatidylinositol glycan anchor biosynthesis class W | 277161 | 278259 | 278311 | 40, 7, 42 |
| 17 | PITPNA | phosphatidylinositol transfer protein alpha | 174238 | 132376 | 167703 | 40, 29, 14 |
| 17 | PITPNC1 | phosphatidylinositol transfer protein, cytoplasmic 1 | 154217 | 197170 | 130935 | 22, 14, 41 |
| 17 | PIP4K2B | phosphatidylinositol-5-phosphate 4-kinase type 2 beta | 276293 | 277791 | 273559 | 26, 3, 8 |
| 17 | PDE6G | phosphodiesterase 6G | 185527 | 182612 | 204237 | 14, 10, 25 |
| 17 | PHOSPHO1 | phosphoethanolamine/phosphocholine phosphatase | 173868 | 108798 | 198740 | 7, 27, 38 |
| 17 | PIRT | phosphoinositide interacting regulator of transient receptor potential channels | 233670 | 263429 | 188803 | 26, 4, 8 |
| 17 | PIK3R5 | phosphoinositide-3-kinase regulatory subunit 5 | 141506 | 276231 | 065320 | |
| 17 | PIK3R6 | phosphoinositide-3-kinase regulatory subunit 6 | 276231 | 185156 | 141506 | 7, 36, 5 |
| 17 | PLCD3 | phospholipase C delta 3 | 161714 | 136448 | 181513 | 3, 4, 9 |
| 17 | PLD6 | phospholipase D family member 6 | 179598 | 133030 | 264187 | 31, 38, 15 |
| 17 | PLD2 | phospholipase D2 | 129219 | 262165 | 141503 | 43, 24, 1 |
| 17 | PRPSAP1 | phosphoribosyl pyrophosphate synthetase associated protein 1 | 161542 | 129646 | 176170 | 29, 38, 4 |
| 17 | PRPSAP2 | phosphoribosyl pyrophosphate synthetase associated protein 2 | 141127 | 171928 | 154025 | 41, 43, 29 |
| 17 | PFAS | phosphoribosylformylglycinamidine synthase | 178921 | 178971 | 125434 | 24, 12, 21 |
| 17 | PRCD | photoreceptor disc component | 214140 | 161544 | 070731 | |
| 17 | PIPOX | pipecolic acid and sarcosine oxidase | 179761 | 109118 | 063015 | 19, 17, 25 |
| 17 | PITPNM3 | PITPNM family member 3 | 091622 | 129195 | 198920 | 9, 36, 25 |
| 17 | PAFAH1B1 | platelet activating factor acetylhydrolase 1b regulatory subunit 1 | 007168 | 127804 | 132361 | 8, 6, 42 |
| 17 | PECAM1 | platelet and endothelial cell adhesion molecule 1 | 261371 | 136478 | 271605 | 43, 32, 45 |
| 17 | PLEKHM1 | pleckstrin homology and RUN domain containing M1 | 225190 | 159314 | 263715 | 43, 24, 4 |
| 17 | PLEKHH3 | pleckstrin homology, MyTH4 and FERM domain containing H3 | 068137 | 037042 | 184451 | 6, 14, 26 |
| 17 | PLXDC1 | plexin domain containing 1 | 161381 | 204952 | 141748 | |
| 17 | PCGF2 | polycomb group ring finger 2 | 277258 | 277972 | 277791 | 34, 14, 6 |
| 17 | PTRF | polymerase I and transcript release factor | 177469 | 168610 | 033627 | 45, 36, 42 |
| 17 | PGAP3 | post-GPI attachment to proteins 3 | 161395 | 141744 | 141736 | 24, 36, 40 |
| 17 | KCTD11 | potassium channel tetramerization domain containing 11 | 213859 | 072818 | 182896 | 37, 26, 45 |
| 17 | KCTD2 | potassium channel tetramerization domain containing 2 | 180901 | 167862 | 281674 | 14, 33, 1 |
| 17 | KCNAB3 | potassium voltage-gated channel subfamily A regulatory beta subunit 3 | 170049 | 281523 | 262730 | 26, 14, 27 |
| 17 | KCNH4 | potassium voltage-gated channel subfamily H member 4 | 089558 | 108774 | 161610 | 41, 13, 5 |
| 17 | KCNH6 | potassium voltage-gated channel subfamily H member 6 | 173826 | 264813 | 136485 | 39, 17, 14 |
| 17 | KCNJ12 | potassium voltage-gated channel subfamily J member 12 | 184185 | 034152 | 212719 | 14, 42, 25 |
| 17 | KCNJ16 | potassium voltage-gated channel subfamily J member 16 | 153822 | 108984 | 123700 | 29, 40, 17 |
| 17 | KCNJ18 | potassium voltage-gated channel subfamily J member 18 | 260458 | 212719 | 283566 | |
| 17 | KCNJ2 | potassium voltage-gated channel subfamily J member 2 | 123700 | 153822 | 125398 | 13, 29, 18 |
| 17 | PRPF8 | pre-mRNA processing factor 8 | 174231 | 167705 | 185561 | 18, 43, 23 |
| 17 | PFN1 | profilin 1 | 108518 | 108523 | 108515 | 41, 37, 27 |
| 17 | PHB | prohibitin | 167085 | 198740 | 281126 | 19, 21, 9 |
| 17 | PRR11 | proline rich 11 | 068489 | 182628 | 265303 | 39, 21, 22 |
| 17 | PRR15L | proline rich 15 like | 167183 | 108439 | 108465 | 37, 16, 33 |
| 17 | PRR29 | proline rich 29 | 224383 | 007314 | 108622 | 43, 42, 38 |
| 17 | PELP1 | proline, glutamate and leucine rich protein 1 | 141456 | 161905 | 141480 | 21, 24, 12 |
| 17 | P3H4 | prolyl 3-hydroxylase family member 4 (non-enzymatic) | 141696 | 173801 | 141756 | 3, 10, 23 |
| 17 | P4HB | prolyl 4-hydroxylase subunit beta | 185624 | 182676 | 141522 | 45, 36, 42 |
| 17 | PTGES3L | prostaglandin E synthase 3 like | 267060 | 266967 | 198863 | |
| 17 | PRAC1 | prostate cancer susceptibility candidate 1 | 159182 | 170689 | 229637 | |
| 17 | PRAC2 | prostate cancer susceptibility candidate 2 | 229637 | 159182 | 159184 | |
| 17 | PSMC5 | proteasome 26S subunit, ATPase 5 | 087191 | 108592 | 108604 | 30, 34, 1 |
| 17 | PSMD11 | proteasome 26S subunit, non-ATPase 11 | 108671 | 010244 | 176749 | 30, 4, 26 |
| 17 | PSMD12 | proteasome 26S subunit, non-ATPase 12 | 197170 | 198265 | 154217 | 1, 18, 26 |
| 17 | PSMD3 | proteasome 26S subunit, non-ATPase 3 | 108344 | 167914 | 108342 | 44, 21, 2 |
| 17 | PSME3 | proteasome activator subunit 3 | 131467 | 126581 | 131480 | |
| 17 | PSMB3 | proteasome subunit beta 3 | 277791 | 277258 | 276293 | 29, 10, 31 |
| 17 | PSMB6 | proteasome subunit beta 6 | 142507 | 182327 | 262165 | 34, 9, 3 |
| 17 | PROCA1 | protein interacting with cyclin A1 | 167525 | 109111 | 109113 | 26, 38, 12 |
| 17 | PRKCA | protein kinase C alpha | 154229 | 091583 | 075429 | 6, 17, 10 |
| 17 | PRKAR1A | protein kinase cAMP-dependent type I regulatory subunit alpha | 108946 | 070540 | 108950 | 42, 39, 8 |
| 17 | PPP1R1B | protein phosphatase 1 regulatory inhibitor subunit 1B | 131771 | 171532 | 131748 | 37, 16, 3 |
| 17 | PPP1R27 | protein phosphatase 1 regulatory subunit 27 | 182676 | 225663 | 185624 | 18, 15, 25 |
| 17 | PPP1R9B | protein phosphatase 1 regulatory subunit 9B | 108819 | 167100 | 108823 | 34, 21, 9 |
| 17 | PPM1D | protein phosphatase, Mg2+/Mn2+ dependent 1D | 170836 | 062725 | 281267 | 39, 38, 41 |
| 17 | PPM1E | protein phosphatase, Mg2+/Mn2+ dependent 1E | 175175 | 108384 | 108395 | 38, 8, 28 |
| 17 | PSMC3IP | PSMC3 interacting protein | 131470 | 108788 | 141699 | 35, 44, 20 |
| 17 | PTGES3L- | PTGES3L-AARSD1 readthrough | 108825 | 131482 | 267060 | 13, 25, 41 |
| | AARSD1 | | | | | |
| 17 | P2RX1 | purinergic receptor P2X1 | 108405 | 004660 | 074370 | 43, 5, 36 |
| 17 | P2RX5 | purinergic receptor P2X5 | 083454 | 127774 | 083457 | 9, 11, 6 |
| 17 | PNPO | pyridoxamine 5′-phosphate oxidase | 108439 | 167182 | 167183 | 24, 42, 27 |
| 17 | PYCR1 | pyrroline-5-carboxylate reductase 1 | 183010 | 197063 | 185105 | 21, 37, 3 |
| 17 | PDK2 | pyruvate dehydrogenase kinase 2 | 005882 | 005884 | 167100 | 15, 17, 8 |
| 17 | RILP | Rab interacting lysosomal protein | 167705 | 074660 | 174231 | 9, 41, 25 |
| 17 | RAB11FIP4 | RAB11 family interacting protein 4 | 131242 | 126860 | 172301 | 31, 27, 38 |
| 17 | RAB34 | RAB34, member RAS oncogene family | 109113 | 167525 | 198242 | 34, 43, 13 |
| 17 | RAB37 | RAB37, member RAS oncogene family | 172794 | 186407 | 186074 | 5, 7, 39 |
| 17 | RAB40B | RAB40B, member RAS oncogene family | 141542 | 141580 | 141560 | 23, 8, 31 |
| 17 | RAB5C | RAB5C, member RAS oncogene family | 108774 | 260325 | 089558 | 18, 2, 3 |
| 17 | RABEP1 | rabaptin, RAB GTPase binding effector protein 1 | 029725 | 161929 | 108559 | 26, 22, 23 |
| 17 | RPH3AL | rabphilin 3A-like (without C2 domains) | 181031 | 272636 | 187624 | 36, 28, 1 |
| 17 | RAD51C | RAD51 paralog C | 108384 | 121101 | 175175 | 30, 35, 3 |
| 17 | RAD51D | RAD51 paralog D | 185379 | 267618 | 073598 | 3, 9, 5 |
| 17 | RDM1 | RAD52 motif containing 1 | 278023 | 271503 | 275722 | 22, 3, 7 |
| 17 | RSAD1 | radical S-adenosyl methionine domain containing 1 | 136444 | 136457 | 136449 | 29, 1, 40 |
| 17 | RANGRF | RAN guanine nucleotide release factor | 108961 | 125434 | 198844 | 29, 22, 41 |
| 17 | RAPGEFL1 | Rap guanine nucleotide exchange factor like 1 | 108352 | 108349 | 171475 | 24, 4, 18 |
| 17 | RAP1GAP2 | RAP1 GTPase activating protein 2 | 132359 | 132361 | 262628 | |
| 17 | RHOT1 | ras homolog family member T1 | 126858 | 185158 | 141314 | 34, 29, 7 |
| 17 | RASL10B | RAS like family 10 member B | 270885 | 270647 | 270765 | 31, 34, 29 |
| 17 | RASD1 | ras related dexamethasone induced 1 | 108551 | 141026 | 133027 | 31, 28, 26 |
| 17 | RAC3 | ras-related C3 botulinum toxin substrate 3 (rho family, small GTP binding protein | 169750 | 169683 | 169738 | 34, 26, 2 |
| | | Rac3) | | | | |
| 17 | RAMP2 | receptor activity modifying protein 2 | 131477 | 108799 | 131475 | 40, 36, 14 |
| 17 | RCVRN | recoverin | 109047 | 065325 | 007237 | 14, 25, 27 |
| 17 | RECQL5 | RecQ like helicase 5 | 108469 | 266714 | 204323 | 43, 21, 18 |
| 17 | RFLNB | refilin B | 183688 | 187624 | 141252 | 34, 6, 7 |
| 17 | RGS9 | regulator of G-protein signaling 9 | 108370 | 120063 | 168646 | 28, 26, 31 |
| 17 | RPTOR | regulatory associated protein of MTOR complex 1 | 141564 | 171246 | 176108 | 22, 14, 6 |
| 17 | RPA1 | replication protein A1 | 132383 | 186532 | 185924 | 41, 40, 3 |
| 17 | RPRML | reprimo like | 179673 | 262633 | 004897 | 14, 8, 38 |
| 17 | RTN4RL1 | reticulon 4 receptor like 1 | 185924 | 132383 | 108963 | 3, 6, 14 |
| 17 | RAH | retinoic acid induced 1 | 108557 | 133027 | 072310 | 18, 16, 40 |
| 17 | RARA | retinoic acid receptor alpha | 131759 | 094804 | 183153 | 23, 42, 13 |
| 17 | RFNG | RFNG O-fucosylpeptide 3-beta-N-acetylglucosaminyltransferase | 169733 | 169738 | 169727 | 36, 31, 37 |
| 17 | RND2 | Rho family GTPase 2 | 108830 | 108828 | 012048 | 31, 29, 8 |
| 17 | ARHGDIA | Rho GDP dissociation inhibitor alpha | 141522 | 185624 | 183684 | 34, 35, 21 |
| 17 | ARHGAP23 | Rho GTPase activating protein 23 | 275832 | 274211 | 277363 | 34, 24, 26 |
| 17 | ARHGAP27 | Rho GTPase activating protein 27 | 159314 | 006062 | 225190 | 22, 36, 16 |
| 17 | ARHGAP44 | Rho GTPase activating protein 44 | 006740 | 141052 | 006744 | 26, 21, 8 |
| 17 | ARHGEF15 | Rho guanine nucleotide exchange factor 15 | 198844 | 108961 | 184650 | 43, 2, 15 |
| 17 | RHBDF2 | rhomboid 5 homolog 2 | 129667 | 129673 | 161544 | 24, 26, 22 |
| 17 | RHBDL3 | rhomboid like 3 | 141314 | 126858 | 108666 | 14, 22, 20 |
| 17 | RNASEK | ribonuclease K | 219200 | 108839 | 161939 | 31, 29, 2 |
| 17 | RPL19 | ribosomal protein L19 | 108298 | 067191 | 141750 | 18, 43, 11 |
| 17 | RPL23 | ribosomal protein L23 | 125691 | 275489 | 002834 | 43, 33, 12 |
| 17 | RPL23A | ribosomal protein L23a | 198242 | 109113 | 160606 | 41, 29, 3 |
| 17 | RPL26 | ribosomal protein L26 | 161970 | 263809 | 189051 | |
| 17 | RPL27 | ribosomal protein L27 | 131469 | 281079 | 068079 | 45, |
| 17 | RPL38 | ribosomal protein L38 | 172809 | 069188 | 141540 | 7, 27, 43 |
| 17 | RPS6KB1 | ribosomal protein S6 kinase B1 | 108443 | 108423 | 267318 | |
| 17 | RFFL | ring finger and FYVE like domain containing E3 ubiquitin protein ligase | 092871 | 005156 | 267618 | 21, 22, 42 |
| 17 | RNF112 | ring finger protein 112 | 128482 | 166482 | 142494 | 14, 25, 8 |
| 17 | RNF135 | ring finger protein 135 | 181481 | 184060 | 196712 | 34, 29, 2 |
| 17 | RNF157 | ring finger protein 157 | 141576 | 129654 | 185262 | 14, 26, 38 |
| 17 | RNF167 | ring finger protein 167 | 108523 | 108528 | 108518 | 7, 9, 6 |
| 17 | RNF213 | ring finger protein 213 | 173821 | 181045 | 173818 | 23, 26, 36 |
| 17 | RNF222 | ring finger protein 222 | 189051 | 161970 | 166579 | 13, 20, |
| 17 | RNF43 | ring finger protein 43 | 108375 | 213246 | 176160 | |
| 17 | RNFT1 | ring finger protein, transmembrane 1 | 189050 | 267318 | 068097 | 38, 34, 7 |
| 17 | RBFOX3 | RNA binding protein, fox-1 homolog 3 | 167281 | 167280 | 182156 | |
| 17 | POLR2A | RNApolymerase II subunit A | 181222 | 259224 | 239697 | 18, 23, 43 |
| 17 | RNASEK- | RNASEK-C17orf49 readthrough | 161939 | 219200 | 258315 | |
| | C17orf49 | | | | | |
| 17 | RPAIN | RPA interacting protein | 129197 | 108559 | 108561 | 37, 15, 1 |
| 17 | RUNDC1 | RUN domain containing 1 | 198863 | 267060 | 281079 | 11, 34, 33 |
| 17 | RUNDC3A | RUN domain containing 3A | 108309 | 004939 | 013306 | 31, 8, 26 |
| 17 | STH | saitohin | 256762 | 186868 | 120071 | |
| 17 | SAP30BP | SAP30 binding protein | 161526 | 259120 | 132470 | 34, 30, 24 |
| 17 | SGCA | sarcoglycan alpha | 108823 | 108819 | 108821 | 25, 43, 15 |
| 17 | SCARF1 | scavenger receptor class F member 1 | 074660 | 167703 | 167705 | 26, 11, 45 |
| 17 | SLFN11 | schlafen family member 11 | 172716 | 166750 | 172123 | 25, 1, 7 |
| 17 | SLFN12 | schlafen family member 12 | 172123 | 172716 | 154760 | 43, 45, 17 |
| 17 | SLFN12L | schlafen family member 12 like | 205045 | 154760 | 236320 | 42, 39, 36 |
| 17 | SLFN13 | schlafen family member 13 | 154760 | 172123 | 205045 | 42, 39, 36 |
| 17 | SLFN14 | schlafen family member 14 | 236320 | 205045 | 108733 | 5, |
| 17 | SLFN5 | schlafen family member 5 | 166750 | 164729 | 172716 | 24, 30, 39 |
| 17 | SOST | sderostin | 167941 | 005102 | 108861 | 17, 45, 6 |
| 17 | SCO1 | SCO1, cytochrome c oxidase assembly protein | 133028 | 109063 | 170222 | 6, 13, 21 |
| 17 | SEBOX | SEBOX homeobox | 274529 | 244045 | 273171 | |
| 17 | SEC14L1 | SEC14 like lipid binding 1 | 129657 | 167889 | 184640 | 45, 6, 39 |
| 17 | SCRN2 | secemin 2 | 141295 | 141294 | 189120 | 26, 40, 1 |
| 17 | SECTM1 | secreted and transmembrane 1 | 141574 | 173762 | 182459 | 34, 9, 5 |
| 17 | SCGB1C2 | secretoglobin family 1C member 2 | 268320 | 167693 | 272636 | |
| 17 | SHPK | sedoheptulokinase | 197417 | 262304 | 040531 | 1, 26, 24 |
| 17 | SEZ6 | seizure related 6 homolog | 063015 | 179761 | 196535 | 8, 14, 28 |
| 17 | SENP3-EIF4A1 | SENP3-EIF4A1 readthrough (NMD candidate) | 277957 | 161956 | 161960 | |
| 17 | SEPT4 | septin 4 | 108387 | 108389 | 181013 | 8, 26, 15 |
| 17 | SEPT9 | septin 9 | 184640 | 129657 | 278938 | |
| 17 | SRSF1 | serine and arginine rich splicing factor 1 | 136450 | 266086 | 264364 | 22, 39, 7 |
| 17 | SRSF2 | serine and arginine rich splicing factor 2 | 161547 | 267168 | 092931 | 39, 36, 13 |
| 17 | SCPEP1 | serine carboxypeptidase 1 | 121064 | 121058 | 121057 | 13, 2, 42 |
| 17 | SHMT1 | serine hydroxymethyltransferase 1 | 176974 | 176994 | 214860 | |
| 17 | SRR | serine racemase | 167720 | 070366 | 167721 | 30, 8, 15 |
| 17 | SERPINF1 | serpin family F member 1 | 132386 | 167711 | 186532 | 14, 1, 30 |
| 17 | SERPINF2 | serpin family F member 2 | 167711 | 167716 | 132386 | 19, 17, 36 |
| 17 | SMYD4 | SET and MYND domain containing 4 | 186532 | 132386 | 132383 | 2, 24, 34 |
| 17 | SHBG | sex hormone binding globulin | 129214 | 129245 | 141504 | 19, 14, 38 |
| 17 | STAC2 | SH3 and cysteine rich domain 2 | 141750 | 108298 | 108306 | 14, 1, 33 |
| 17 | SHISA6 | shisa family member 6 | 188803 | 233670 | 007174 | 8, 17, 33 |
| 17 | SDK2 | sidekick cell adhesion molecule 2 | 069188 | 179604 | 172809 | 38, 30, 12 |
| 17 | SPPL2C | signal peptide peptidase like 2C | 185294 | 120088 | 186868 | 38, 8, |
| 17 | SRP68 | signal recognition particle 68 | 167881 | 167880 | 186919 | 41, 35, 13 |
| 17 | STAT3 | signal transducer and activator of transcription 3 | 168610 | 126561 | 177469 | 24, 42, 36 |
| 17 | STAT5A | signal transducer and activator of transcription 5A | 126561 | 173757 | 168610 | 41, 39, 22 |
| 17 | STAT5B | signal transducer and activator of transcription 5B | 173757 | 167925 | 126561 | 31, 22, 32 |
| 17 | SIRT7 | sirtuin 7 | 187531 | 185813 | 197063 | 21, 28, 29 |
| 17 | SLC9A3R1 | SLC9A3 regulator 1 | 109062 | 186074 | 109065 | |
| 17 | SSH2 | slingshot protein phosphatase 2 | 141298 | 167549 | 176927 | 5, 21, 22 |
| 17 | SCIMP | SLP adaptor and CSK interacting membrane protein | 161929 | 180626 | 029725 | 22, 36, 5 |
| 17 | SMURF2 | SMAD specific E3 ubiquitin protein ligase 2 | 108854 | 258890 | 176809 | 37, 43, 45 |
| 17 | SGSM2 | small G protein signaling modulator 2 | 141258 | 167721 | 070444 | 9, 33, 26 |
| 17 | SMIM5 | small integral membrane protein 5 | 204323 | 108469 | 259120 | 17, 6, 40 |
| 17 | SMIM6 | small integral membrane protein 6 | 259120 | 204323 | 161526 | 29, 38, 28 |
| 17 | SUMO2 | small ubiquitin-like modifier 2 | 188612 | 189159 | 125450 | |
| 17 | SMG6 | SMG6, nonsense mediated mRNA decay factor | 070366 | 177374 | 167720 | 43, 39, 18 |
| 17 | SMG8 | SMG8, nonsense mediated mRNA decay factor | 167447 | 265303 | 153982 | 13, 7, 24 |
| 17 | SMCR8 | Smith-Magenis syndrome chromosome region, candidate 8 | 176994 | 177302 | 176974 | 29, 37, 36 |
| 17 | SMTNL2 | smoothelin like 2 | 188176 | 167741 | 161905 | 25, 26, 30 |
| 17 | SNF8 | SNF8, ESCRT-II complex subunit | 159210 | 159202 | 159224 | 22, |
| 17 | SCN4A | sodium voltage-gated channel alpha subunit 4 | 007314 | 007312 | 224383 | 31, 18, 25 |
| 17 | SLC13A2 | solute carrier family 13 member 2 | 007216 | 258472 | 109101 | 42, 17, 16 |
| 17 | SLC13A5 | solute carrier family 13 member 5 | 141485 | 279257 | 132530 | 19, 42, 8 |
| 17 | SLC16A11 | solute carrier family 16 member 11 | 174326 | 174327 | 132514 | 29, 2, 23 |
| 17 | SLC16A13 | solute carrier family 16 member 13 | 174327 | 161940 | 174326 | 7, 24, 23 |
| 17 | SLC16A3 | solute carrier family 16 member 3 | 141526 | 176155 | 141551 | 17, 13, 9 |
| 17 | SLC16A5 | solute carrier family 16 member 5 | 170190 | 167863 | 125449 | 3, 9, 1 |
| 17 | SLC16A6 | solute carrier family 16 member 6 | 108932 | 141337 | 070540 | 35, 9, 2 |
| 17 | SLC2A4 | solute carrier family 2 member 4 | 181856 | 181885 | 006047 | 15, 25, 13 |
| 17 | SLC25A10 | solute carrier family 25 member 10 | 183048 | 262660 | 215644 | 21, 37, 27 |
| 17 | SLC25A11 | solute carrier family 25 member 11 | 108528 | 185245 | 108523 | 34, 19, 25 |
| 17 | SLC25A19 | solute carrier family 25 member 19 | 125454 | 125457 | 177885 | 38, 22, 21 |
| 17 | SLC25A35 | solute carrier family 25 member 35 | 125434 | 178921 | 108961 | 22, 7, 44 |
| 17 | SLC25A39 | solute carrier family 25 member 39 | 013306 | 108309 | 030582 | 3, 9, 5 |
| 17 | SLC26A11 | solute carrier family 26 member 11 | 181045 | 181523 | 173821 | 26, 9, 2 |
| 17 | SLC35B1 | solute carrier family 35 member B1 | 121073 | 121067 | 121104 | |
| 17 | SLC35G3 | solute carrier family 35 member G3 | 164729 | 141161 | 166750 | 38, |
| 17 | SLC35G6 | solute carrier family 35 member G6 | 259224 | 174282 | 181222 | |
| 17 | SLC38A10 | solute carrier family 38 member 10 | 157637 | 224877 | 185332 | 34, 21, 31 |
| 17 | SLC39A11 | solute carrier family 39 member 11 | 133195 | 125398 | 180616 | 34, 24, 35 |
| 17 | SLC4A1 | solute carrier family 4 member 1 (Diego blood group) | 004939 | 108312 | 108309 | 19, 15, 7 |
| 17 | SLC43A2 | solute carrier family 43 member 2 | 167703 | 174238 | 074660 | 32, 36, 28 |
| 17 | SLC46A1 | solute carrier family 46 member 1 | 076351 | 109072 | 258472 | 29, 36, 2 |
| 17 | SLC47A1 | solute carrier family 47 member 1 | 142494 | 128482 | 072210 | 2, 24, 38 |
| 17 | SLC47A2 | solute carrier family 47 member 2 | 180638 | 072210 | 108602 | 13, 17, 3 |
| 17 | SLC5A10 | solute carrier family 5 member 10 | 154025 | 141127 | 188522 | 17, 15, 22 |
| 17 | SLC52A1 | solute carrier family 52 member 1 | 132517 | 129250 | 180787 | 32, 18, 24 |
| 17 | SLC6A4 | solute carrier family 6 member 4 | 108576 | 126653 | 108578 | 32, 24, 16 |
| 17 | SSTR2 | somatostatin receptor 2 | 180616 | 133195 | 166685 | 8, 14, 17 |
| 17 | SNX11 | sorting nexin 11 | 002919 | 108468 | 141293 | 41, 45, 36 |
| 17 | SP2 | Sp2 transcription factor | 167182 | 189120 | 108439 | 43, 9, 3 |
| 17 | SP6 | Sp6 transcription factor | 189120 | 141295 | 167182 | 29, 26, 32 |
| 17 | SPOP | speckle type BTB/POZ protein | 121067 | 182575 | 121073 | 30, 45, 13 |
| 17 | SPDYE4 | speedy/RINGO cell cycle regulator family member E4 | 183318 | 161973 | 185156 | 38, |
| 17 | SPACA3 | sperm acrosome associated 3 | 141316 | 006042 | 108684 | 38, 10, 8 |
| 17 | SPECC1 | sperm antigen with calponin homology and coiled-coil domains 1 | 128487 | 108599 | 170298 | |
| 17 | SPAG5 | sperm associated antigen 5 | 076382 | 109107 | 167524 | 38, 43, 9 |
| 17 | SPAG7 | sperm associated antigen 7 | 091640 | 108515 | 108509 | 4, |
| 17 | SPAG9 | sperm associated antigen 9 | 008294 | 141232 | 239672 | 11, 4, 8 |
| 17 | SPEM1 | spermatid maturation 1 | 181323 | 169992 | 184560 | 38, |
| 17 | SPATA20 | spermatogenesis associated 20 | 006282 | 049283 | 006283 | 38, 7, 3 |
| 17 | SPATA22 | spermatogenesis associated 22 | 141255 | 127780 | 108381 | 38, 8, 32 |
| 17 | SPATA32 | spermatogenesis associated 32 | 184361 | 184922 | 006062 | 38, 8, |
| 17 | SAT2 | spermidine/spermine N1-acetyltransferase family member 2 | 141504 | 129214 | 129244 | 1, 34, 20 |
| 17 | SPNS2 | sphingolipid transporter 2 | 183018 | 182557 | 132382 | 37, 14, 16 |
| 17 | SPNS3 | sphingolipid transporter 3 (putative) | 182557 | 132388 | 183018 | 5, 21, 7 |
| 17 | SPHK1 | sphingosine kinase 1 | 176170 | 161542 | 175931 | 13, 45, 35 |
| 17 | SKA2 | spindle and kinetochore associated complex subunit 2 | 182628 | 108395 | 068489 | 34, 10, 9 |
| 17 | SUPT4H1 | SPT4 homolog, DSIF elongation factor subunit | 213246 | 005379 | 108375 | 1, 34, 6 |
| 17 | SUPT6H | SPT6 homolog, histone chaperone | 109111 | 132581 | 167525 | 43, 20, 16 |
| 17 | SKAP1 | src kinase associated phosphoprotein 1 | 141293 | 002919 | 120094 | 31, 36, 10 |
| 17 | SRCIN1 | SRC kinase signaling inhibitor 1 | 277363 | 275832 | 273604 | 31, 8, 14 |
| 17 | SOX15 | SRY-box 15 | 129194 | 129255 | 129245 | 13, 31, 35 |
| 17 | SOX9 | SRY-box 9 | 125398 | 123700 | 133195 | 11, 38, 26 |
| 17 | ST6GALNAC1 | ST6 N-acetylgalactosaminide alpha-2,6-sialyltransferase 1 | 070526 | 070731 | 182534 | 42, 36, 3 |
| 17 | ST6GALNAC2 | ST6 N-acetylgalactosaminide alpha-2,6-sialyltransferase 2 | 070731 | 214140 | 070526 | 13, 35, 40 |
| 17 | STARD3 | StAR related lipid transfer domain containing 3 | 131748 | 131771 | 173991 | 39, 9, 31 |
| 17 | STRADA | STE20-related kinase adaptor alpha | 266173 | 136490 | 125695 | 36, 45, 38 |
| 17 | SARM1 | sterile alpha and TIR motif containing 1 | 004139 | 273171 | 109072 | 36, 8, 2 |
| 17 | SAMD14 | sterile alpha motif domain containing 14 | 167100 | 005882 | 108819 | 8, 29, 14 |
| 17 | SREBF1 | sterol regulatory element binding transcription factor 1 | 072310 | 108557 | 175662 | 24, 39, 42 |
| 17 | SDF2 | stromal cell derived factor 2 | 132581 | 007202 | 109111 | 34, 27, 43 |
| 17 | SENP3 | SUMO 1/sentrin/SMT3 specific peptidase 3 | 161956 | 161955 | 277957 | 6, 31, 9 |
| 17 | SOCS3 | suppressor of cytokine signaling 3 | 184557 | 204278 | 087157 | 1, 27, 10 |
| 17 | SOCS7 | suppressor of cytokine signaling 7 | 274211 | 277399 | 275832 | 18, 23, 14 |
| 17 | SUZ12 | SUZ12 polycomb repressive complex 2 subunit | 178691 | 108651 | 185158 | 11, 22, 7 |
| 17 | SMARCD2 | SWI/SNF related, matrix associated, actin dependent regulator of chromatin, | 108604 | 087191 | 213218 | 31, 3, 33 |
| | | subfamily d, member 2 | | | | |
| 17 | SMARCE1 | SWI/SNF related, matrix associated, actin dependent regulator of chromatin, | 073584 | 126353 | 264058 | 36, 45, 12 |
| | | subfamily e, member 1 | | | | |
| 17 | SYNGR2 | synaptogyrin 2 | 108639 | 187997 | 167900 | 41, 27, 21 |
| 17 | SYNRG | synergin qamma | 275066 | 276023 | 278053 | 22, 39, 20 |
| 17 | STX8 | syntaxin 8 | 170310 | 065320 | 166596 | 45, 2, 1 |
| 17 | STXBP4 | syntaxin binding protein 4 | 166263 | 166260 | 108924 | 9, 6, 45 |
| 17 | TAC4 | tachykinin 4 (hemokinin) | 176358 | 136504 | 108813 | 18, |
| 17 | TAOK1 | TAO kinase 1 | 160551 | 108256 | 168792 | |
| 17 | TOM1L1 | target of myb1 like 1 membrane trafficking protein | 141198 | 141200 | 166260 | |
| 17 | TOM1L2 | target of myb1 like 2 membrane trafficking protein | 175662 | 072310 | 171962 | 34, 26, 38 |
| 17 | TAF15 | TATA-box binding protein associated factor 15 | 270647 | 006125 | 270885 | 36, 24, 39 |
| 17 | TAX1BP3 | Tax1 binding protein 3 | 213977 | 040531 | 257950 | 34, 19, 1 |
| 17 | TBC1D16 | TBC1 domain family member 16 | 167291 | 141582 | 141519 | 43, 35, 23 |
| 17 | TBC1D26 | TBC1 domain family member 26 | 214946 | 255104 | 170425 | 38, 39, 8 |
| 17 | TBC1D26 | TBC1 domain family member 26 | 255104 | 187607 | 214946 | 38, 39, 8 |
| 17 | TBC1D28 | TBC1 domain family member 28 | 189375 | 213077 | 249459 | 38, |
| 17 | TBC1D29 | TBC1 domain family member 29 | 266733 | 108587 | 176390 | |
| 17 | TBC1D3 | TBC1 domain family member 3 | 274611 | 278599 | 278845 | 18, 24, 16 |
| 17 | TBC1D3B | TBC1 domain family member 3B | 274808 | 275302 | 276085 | 38, 16, 37 |
| 17 | TBC1D3C | TBC1 domain family member 3C | 278299 | 274419 | 278599 | 38, 16, 22 |
| 17 | TBC1D3D | TBC1 domain family member 3D | 274419 | 274512 | 278299 | |
| 17 | TBC1D3E | TBC1 domain family member 3E | 278599 | 278299 | 274611 | |
| 17 | TBC1D3F | TBC1 domain family member 3F | 275954 | 274226 | 273611 | |
| 17 | TBC1D3G | TBC1 domain family member 3G | 260287 | 274933 | 274226 | |
| 17 | TBC1D3H | TBC1 domain family member 3H | 274226 | 260287 | 275954 | 16, 2, 36 |
| 17 | TBC1D3I | TBC1 domain family member 3I | 274933 | 276070 | 260287 | |
| 17 | TBC1D3K | TBC1 domain family member 3K | 273513 | 275410 | 274512 | |
| 17 | TBC1D3L | TBC1 domain family member 3L | 274512 | 273513 | 274419 | 16, 2, 36 |
| 17 | TBKBP1 | TBK1 binding protein 1 | 198933 | 108424 | 073861 | 26, 18, 14 |
| 17 | TBX2 | T-box 2 | 121068 | 141376 | 121075 | |
| 17 | TBX21 | T-box 21 | 073861 | 198933 | 006025 | 36, 22, 28 |
| 17 | TBX4 | T-box 4 | 121075 | 121068 | 253506 | 1, 10, 32 |
| 17 | TEKT1 | tektin 1 | 167858 | 177294 | 108839 | 42, 30, 26 |
| 17 | TEKT3 | tektin 3 | 125409 | 109099 | 239704 | 38, 42, 10 |
| 17 | TEN1 | TEN1, CST complex subunit | 257949 | 161533 | 261408 | 34, 4, 9 |
| 17 | TEN1-CDK3 | TEN1-CDK3 readthrough (NMD candidate) | 261408 | 257949 | 250506 | 22, 23, 16 |
| 17 | TNS4 | tensin 4 | 131746 | 141753 | 126353 | 4, 24, 40 |
| 17 | TEPSIN | TEPSIN, adaptor related protein complex 4 accessory protein | 167302 | 141577 | 224877 | |
| 17 | TEX14 | testis expressed 14, intercellular bridge forming factor | 121101 | 181013 | 108384 | 38, 10, 22 |
| 17 | TEX19 | testis expressed 19 | 182459 | 141574 | 181408 | 38, 21, 3 |
| 17 | TEX2 | testis expressed 2 | 136478 | 178607 | 261371 | 38, 24, 42 |
| 17 | TSPAN10 | tetraspanin 10 | 182612 | 182446 | 185527 | |
| 17 | TTC19 | tetratricopeptide repeat domain 19 | 011295 | 214941 | 141027 | 29, 15, 31 |
| 17 | TTC25 | tetratricopeptide repeat domain 25 | 204815 | 131473 | 173786 | 30, 9, 6 |
| 17 | TANC2 | tetratricopeptide repeat, ankyrin repeat and coiled-coil containing 2 | 170921 | 173838 | 008283 | 18, 26, 31 |
| 17 | TIAF1 | TGFB1-induced anti-apoptotic factor 1 | 221995 | 196535 | 108255 | 13, 15, 17 |
| 17 | TXNDC17 | thioredoxin domain containing 17 | 129235 | 282936 | 108590 | 43, 33, 34 |
| 17 | TK1 | thymidine kinase 1 | 167900 | 108639 | 183077 | 21, 41, 23 |
| 17 | THRA | thyroid hormone receptor, alpha | 126351 | 008838 | 126368 | 34, 11, 8 |
| 17 | TIMP2 | TIMP metallopeptidase inhibitor 2 | 035862 | 055483 | 178404 | 11, 15, 32 |
| 17 | TCAP | titin-cap | 173991 | 131748 | 141744 | 25, 15, 26 |
| 17 | TLCD1 | TLC domain containing 1 | 160606 | 198242 | 160602 | 17, 20, |
| 17 | TLCD2 | TLC domain containing 2 | 185561 | 174231 | 167716 | 34, 14, 24 |
| 17 | TMEM256- | TMEM256-PLSCR3 readthrough (NMD candidate) | 187838 | 174292 | 262481 | 13, 45, 26 |
| | PLSCR3 | | | | | |
| 17 | TMEM256- | TMEM256-PLSCR3 readthrough (NMD candidate) | 262481 | 187838 | 205544 | |
| | PLSCR3 | | | | | |
| 17 | TNFAIP1 | TNF alpha induced protein 1 | 109079 | 109083 | 004142 | 43, 34, 25 |
| 17 | TRAF4 | TNF receptor associated factor 4 | 076604 | 160602 | 173065 | 37, 26, 12 |
| 17 | TNFRSF13B | TNF receptor superfamily member 13B | 240505 | 266302 | 133030 | 36, 21, 39 |
| 17 | TNFSF12- | TNFSF12-TNFSF13 readthrough | 248871 | 239697 | 161955 | |
| | TNFSF13 | | | | | |
| 17 | TOP2A | topoisomerase (DNA) II alpha | 131747 | 183153 | 141753 | |
| 17 | TOP3A | topoisomerase (DNA) III alpha | 177302 | 177427 | 176994 | 41, 39, 23 |
| 17 | TLK2 | tousled like kinase 2 | 146872 | 087995 | 011028 | 41, 38, 12 |
| 17 | TRAPPC1 | trafficking protein particle complex 1 | 170043 | 262730 | 170037 | 34, 41, 27 |
| 17 | TEFM | transcription elongation factor, mitochondrial | 172171 | 176208 | 184060 | 29, 34, 26 |
| 17 | TADA2A | transcriptional adaptor 2A | 276234 | 278505 | 276023 | 30, 5, 9 |
| 17 | TOB1 | transducer of ERBB2,1 | 141232 | 173714 | 008294 | 2, 4, 7 |
| 17 | TVP23B | trans-golgi network vesicle protein 23 homolog B | 171928 | 171931 | 141127 | 4, 30, 31 |
| 17 | TVP23C | trans-golgi network vesicle protein 23 homolog C | 175106 | 259024 | 241322 | 31, 29, 37 |
| 17 | TRPV1 | transient receptor potential cation channel subfamily V member 1 | 196689 | 167723 | 262304 | 27, 24, 30 |
| 17 | TRPV2 | transient receptor potential cation channel subfamily V member 2 | 187688 | 170315 | 181350 | 7, 22, 35 |
| 17 | TRPV3 | transient receptor potential cation channel subfamily V member 3 | 167723 | 108381 | 196689 | 29, 25, 38 |
| 17 | TACO1 | translational activator of cytochrome c oxidase I | 136463 | 136485 | 198909 | 41, 3, 18 |
| 17 | TIMM22 | translocase of inner mitochondrial membrane 22 | 177370 | 167693 | 159842 | 27, 37, 1 |
| 17 | TM4SF5 | transmembrane 4 L six family member 5 | 142484 | 141497 | 182853 | 29, 17, 15 |
| 17 | TMIGD1 | transmembrane and immunoglobulin domain containing 1 | 182271 | 108578 | 108582 | 17, 14, 16 |
| 17 | TMUB2 | transmembrane and ubiquitin like domain containing 2 | 168591 | 161664 | 087152 | 5, 23, 1 |
| 17 | TMC6 | transmembrane channel like 6 | 141524 | 078687 | 167895 | 41, 5, 36 |
| 17 | TMC8 | transmembrane channel like 8 | 167895 | 141524 | 187997 | 39, 41, 22 |
| 17 | TMEM100 | transmembrane protein 100 | 166292 | 261873 | 141179 | 44, |
| 17 | TMEM101 | transmembrane protein 101 | 091947 | 161653 | 161654 | 21, 9, 25 |
| 17 | TMEM102 | transmembrane protein 102 | 181284 | 283439 | 161958 | 21, 2, 13 |
| 17 | TMEM104 | transmembrane protein 104 | 109066 | 109065 | 161509 | 26, 31, 6 |
| 17 | TMEM105 | transmembrane protein 105 | 185332 | 157637 | 266074 | |
| 17 | TMEM106A | transmembrane protein 106A | 184988 | 188554 | 236383 | 39, 36, 17 |
| 17 | TMEM107 | transmembrane protein 107 | 179029 | 220205 | 196544 | 7, 5, 29 |
| 17 | TMEM11 | transmembrane protein 11 | 178307 | 109016 | 274180 | 29, 40, 17 |
| 17 | TMEM132E | transmembrane protein 132E | 181291 | 108702 | 132141 | 2, 15, 33 |
| 17 | TMEM199 | transmembrane protein 199 | 244045 | 004142 | 274529 | 29, 1, 37 |
| 17 | TMEM220 | transmembrane protein 220 | 187824 | 170222 | 263429 | 13, 33, 2 |
| 17 | TMEM235 | transmembrane protein 235 | 204278 | 089685 | 184557 | 8, 12, |
| 17 | TMEM256 | transmembrane protein 256 | 205544 | 262481 | 169992 | 3, 7, 41 |
| 17 | TMEM88 | transmembrane protein 88 | 167874 | 132510 | 183011 | 32, 36, 25 |
| 17 | TMEM92 | transmembrane protein 92 | 167105 | 108821 | 015532 | 37, 16, 9 |
| 17 | TMEM94 | transmembrane protein 94 | 177728 | 177885 | 177303 | 43, 26, 8 |
| 17 | TMEM95 | transmembrane protein 95 | 182896 | 213859 | 174292 | 24, 38, |
| 17 | TMEM97 | transmembrane protein 97 | 109084 | 087095 | 109083 | 38, 4, 9 |
| 17 | TMEM98 | transmembrane protein 98 | 006042 | 176658 | 141316 | 14, 31, 32 |
| 17 | TMEM99 | transmembrane protein 99 | 167920 | 186395 | 187242 | 6, 41, 23 |
| 17 | TNRC6C | trinucleotide repeat containing 60 | 078687 | 278938 | 141524 | 22, 18, 29 |
| 17 | TRIM16 | tripartite motif containing 16 | 221926 | 251537 | 187607 | 24, 32, |
| 17 | TRIM16L | tripartite motif containing 16-like | 108448 | 249459 | 171931 | 13, 34, 20 |
| 17 | TRIM25 | tripartite motif containing 25 | 121060 | 153933 | 121058 | 22, 9, 1 |
| 17 | TRIM37 | tripartite motif containing 37 | 108395 | 175175 | 182628 | 38, 8, 31 |
| 17 | TRIM47 | tripartite motif containing 47 | 132481 | 132471 | 141569 | 34, 29, 26 |
| 17 | TRIM65 | tripartite motif containing 65 | 141569 | 132481 | 204316 | 41, 21, 11 |
| 17 | TSEN54 | tRNA splicing endonuclease subunit 54 | 182173 | 177303 | 073350 | 37, 9, 25 |
| 17 | TSPOAP1 | TSPO associated protein 1 | 005379 | 005381 | 213246 | |
| 17 | TSR1 | TSR1, ribosome maturation factor | 167721 | 167720 | 141258 | 42, 3, 24 |
| 17 | TUBD1 | tubulin delta 1 | 108423 | 062716 | 108443 | 29, 28, 1 |
| 17 | TBCD | tubulin folding cofactor D | 141556 | 167363 | 141579 | 14, 44, 40 |
| 17 | TUBG1 | tubulin gamma 1 | 131462 | 141699 | 037042 | 30, 18, 38 |
| 17 | TUBG2 | tubulin gamma 2 | 037042 | 131462 | 068137 | 36, 8, 24 |
| 17 | TTLL6 | tubulin tyrosine ligase like 6 | 170703 | 159184 | 136436 | |
| 17 | TNFSF12 | tumor necrosis factor superfamily member 12 | 239697 | 181222 | 248871 | |
| 17 | TNFSF13 | tumor necrosis factor superfamily member 13 | 161955 | 248871 | 161956 | 27, 5, 17 |
| 17 | TP53 | tumor protein p53 | 141510 | 129244 | 141499 | 24, 45, 4 |
| 17 | TP53I13 | tumor protein p53 inducible protein 13 | 167543 | 168792 | 108262 | |
| 17 | TUSC5 | tumor suppressor candidate 5 | 184811 | 205899 | 108953 | 1, 6, 25 |
| 17 | TVP23C-CDRT4 | TVP23C-CDRT4 readthrough | 259024 | 239704 | 175106 | |
| 17 | TTYH2 | tweety family member 2 | 141540 | 172809 | 171595 | 8, 1, 38 |
| 17 | YWHAE | tyrosine 3-monooxygenase/tryptophan 5-monooxygenase activation protein | 108953 | 184811 | 167193 | 45, 13, 15 |
| | | epsilon | | | | |
| 17 | TNK1 | tyrosine kinase non receptor 1 | 174292 | 182896 | 187838 | 24, 33, 44 |
| 17 | UBALD2 | UBA like domain containing 2 | 185262 | 141576 | 129646 | 6, 29, 22 |
| 17 | UBB | ubiquitin B | 170315 | 166582 | 187688 | |
| 17 | UBE2G1 | ubiquitin conjugating enzyme E2 G1 | 132388 | 185722 | 182557 | 22, 29, 11 |
| 17 | UBE20O | ubiquitin conjugating enzyme E2 O | 175931 | 176170 | 129673 | 35, 33, 18 |
| 17 | UBE2Z | ubiquitin conjugating enzyme E2 Z | 159202 | 159199 | 159210 | 29, 40, 9 |
| 17 | USP22 | ubiquitin specific peptidase 22 | 124422 | 205212 | 109016 | 22, 35, 9 |
| 17 | USP32 | ubiquitin specific peptidase 32 | 170832 | 167434 | 141371 | 23, 34, 29 |
| 17 | USP36 | ubiquitin specific peptidase 36 | 055483 | 108669 | 035862 | 23, 43, 24 |
| 17 | USP43 | ubiquitin specific peptidase 43 | 154914 | 166596 | 184544 | 1, 26, 32 |
| 17 | USP6 | ubiquitin specific peptidase 6 | 129204 | 167840 | 180626 | 22, 38, 3 |
| 17 | B3GNTL1 | UDP-GlcNAc: betaGal beta-1,3-N-acetylglucosaminyltransferase-like 1 | 175711 | 141579 | 176845 | 9, 5, 36 |
| 17 | UNC119 | unc-119 lipid binding chaperone | 109103 | 109101 | 087111 | 14, 34, 22 |
| 17 | UNC13D | unc-13 homolog D | 092929 | 132478 | 132471 | 37, 5, 22 |
| 17 | UNC45B | unc-45 myosin chaperone B | 141161 | 073536 | 164729 | 15, 18, 25 |
| 17 | ULK2 | unc-51 like autophagy activating kinase 2 | 083290 | 108602 | 108599 | 11, 38, 22 |
| 17 | SGK494 | Uncharacterized serine/threonine-protein kinase SgK494 | 167524 | 076382 | 007202 | 40, 18, 24 |
| 17 | UNK | unkempt family zinc finger | 132478 | 132475 | 092929 | 14, 36, 12 |
| 17 | UBTF | upstream binding transcription factor, RNA polymerase I | 108312 | 087152 | 004939 | 41, 33, 39 |
| 17 | UTS2R | urotensin 2 receptor | 181408 | 182459 | 181396 | 27, 38, |
| 17 | USH1G | USH1 protein network component sans | 182040 | 172782 | 183034 | 24, 8, 38 |
| 17 | UTP18 | UTP18, small subunit processome component | 011260 | 011258 | 154975 | 10, 22, 29 |
| 17 | UTP6 | UTP6, small subunit processome component | 108651 | 172301 | 178691 | 10, 43, 45 |
| 17 | VPS25 | vacuolar protein sorting 25 homolog | 131475 | 131477 | 126562 | 34, 30, 6 |
| 17 | VMP1 | vacuole membrane protein 1 | 062716 | 141378 | 108423 | 5, 40, 23 |
| 17 | VEZF1 | vascular endothelial zinc finger 1 | 136451 | 180891 | 266086 | 1, 14, 6 |
| 17 | VAT1 | vesicle amine transport 1 | 108828 | 068079 | 108830 | 35, 2, 39 |
| 17 | VAMP2 | vesicle associated membrane protein 2 | 220205 | 263620 | 179029 | 26, 14, 40 |
| 17 | VMO1 | vitelline membrane outer layer 1 homolog | 182853 | 142484 | 182327 | 31, 20, 6 |
| 17 | VTN | vitronectin | 109072 | 004139 | 076351 | 19, 23, 2 |
| 17 | VPS53 | VPS53, GARP complex subunit | 141252 | 183688 | 167695 | |
| 17 | WFIKKN2 | WAP, follistatin/kazal, immunoglobulin, kunitz and netrin domain containing 2 | 173714 | 108848 | 141232 | 24, 8, 37 |
| 17 | WIPF2 | WAS/WASL interacting protein family member 2 | 171475 | 108352 | 094804 | |
| 17 | WSB1 | WD repeat and SOCS box containing 1 | 109046 | 256618 | 141068 | 39, 42, 11 |
| 17 | WRAP53 | WD repeat containing antisense to TP53 | 141499 | 141510 | 108947 | 39, 24, 9 |
| 17 | WDR45B | WD repeat domain 45B | 141580 | 141568 | 141542 | 37, 29, 9 |
| 17 | WDR81 | WD repeat domain 81 | 167716 | 185561 | 167711 | 24, 22, 1 |
| 17 | WIPI1 | WD repeat domain, phosphoinositide interacting 1 | 070540 | 108932 | 108946 | 43, 32, 29 |
| 17 | WNK4 | WNK lysine deficient protein kinase 4 | 126562 | 131475 | 183978 | 17, 16, 23 |
| 17 | WNT3 | Wnt family member 3 | 108379 | 073969 | 158955 | 37, 29, 15 |
| 17 | WNT9B | Wnt family member 9B | 158955 | 108379 | 108433 | 11, 27, 25 |
| 17 | WSCD1 | WSC domain containing 1 | 179314 | 091592 | 129221 | |
| 17 | WBP2 | WW domain binding protein 2 | 132471 | 092929 | 132481 | 2, 8, 39 |
| 17 | XAF1 | XIAP associated factor 1 | 132530 | 141485 | 177294 | 36, 13, 4 |
| 17 | XYLT2 | xylosyltransferase 2 | 015532 | 167105 | 108826 | 35, 3, 22 |
| 17 | YBX2 | Y-box binding protein 2 | 006047 | 181856 | 132507 | 38, 18, 1 |
| 17 | YPEL2 | yippee like 2 | 175155 | 153982 | 108406 | 30, 26, 22 |
| 17 | ZFP3 | ZFP3 zinc finger protein | 180787 | 132517 | 167840 | 18, 26, 29 |
| 17 | ZACN | zinc activated ion channel | 186919 | 167881 | 182687 | 39, 15, 10 |
| 17 | ZBTB4 | zinc finger and BTB domain containing 4 | 174282 | 170175 | 259224 | 29, 34, 14 |
| 17 | ZNHIT3 | zinc finger HIT-type containing 3 | 273611 | 275954 | 278259 | 34, 15, 43 |
| 17 | ZMYND15 | zinc finger MYND-type containing 15 | 141497 | 161921 | 142484 | 38, 3, 30 |
| 17 | ZNF18 | zinc finger protein 18 | 154957 | 007174 | 065559 | 18, 13, 29 |
| 17 | ZNF207 | zinc finger protein 207 | 010244 | 108666 | 108671 | |
| 17 | ZNF232 | zinc finger protein 232 | 167840 | 180787 | 129204 | 13, 5, 44 |
| 17 | ZNF286A | zinc finger protein 286A | 187607 | 221926 | 255104 | 11, 41, 15 |
| 17 | ZNF286B | zinc finger protein 286B | 249459 | 189375 | 108448 | 12, 22, 14 |
| 17 | ZNF287 | zinc finger protein 287 | 141040 | 181350 | 197566 | 45, 22, 17 |
| 17 | ZNF385C | zinc finger protein 385C | 187595 | 168256 | 267221 | 14, 38, 12 |
| 17 | ZNF594 | zinc finger protein 594 | 180626 | 129204 | 161929 | 1, 15, 40 |
| 17 | ZNF624 | zinc finger protein 624 | 197566 | 141040 | 170160 | 24, 9, 17 |
| 17 | ZNF652 | zinc finger protein 652 | 198740 | 173868 | 167085 | |
| 17 | ZNF750 | zinc finger protein 750 | 141579 | 141556 | 175711 | 24, 42, 39 |
| 17 | ZNF830 | zinc finger protein 830 | 198783 | 132141 | 005156 | 6, |
| 17 | ZSWIM7 | zinc finger SWIM-type containing 7 | 214941 | 170425 | 011295 | 6, 34, 30 |
| 17 | ZZEF1 | zinc finger ZZ-type and EF-hand domain containing 1 | 074755 | 074370 | 167740 | 43, 22, 36 |
| 17 | ZPBP2 | zona pellucida binding protein 2 | 186075 | 161405 | 073605 | 38, 8 |

Table 8 provides the gene information for the genes found on Chromosomes 18-19.

TABLE 8
Genes on Chromosomes 18-19

| | | | | Up | Down | Tissue |
|---|---|---|---|---|---|---|
| Chr. | Gene Symbol | Gene Name | ENSG | ENSG | ENSG | Code |
| 18 | KDSR | 3-ketodihydrosphingosine reductase | 119537 | 171791 | 119541 | 10, 1, 42 |
| 18 | ABHD3 | abhydrolase domain containing 3 | 158201 | 167088 | 101752 | 1, 26, 37 |
| 18 | ACAA2 | acetyl-CoA acyltransferase 2 | 167315 | 101670 | 266997 | 21, 17, 15 |
| 18 | ASXL3 | additional sex combs like 3, transcriptional regulator | 141431 | 166960 | 101746 | 38, 28, 9 |
| 18 | ADCYAP1 | adenylate cyclase activating polypeptide 1 | 141433 | 176105 | 101574 | |
| 18 | ADNP2 | ADNP homeobox 2 | 101544 | 101546 | 178184 | 22, 12, 9 |
| 18 | AFG3L2 | AFG3 like matrix AAA peptidase subunit 2 | 141385 | 176014 | 141391 | 35, 21, 1 |
| 18 | AKAIN1 | A-kinase anchor inhibitor 1 | 231824 | 170579 | 198081 | |
| 18 | ALPK2 | alpha kinase 2 | 198796 | 049759 | 172175 | 18, 9, 17 |
| 18 | ANKRD12 | ankyrin repeat domain 12 | 101745 | 178127 | 128791 | 36, 39, 22 |
| 18 | ANKRD29 | ankyrin repeat domain 29 | 154065 | 141458 | 053747 | 5, 17, |
| 18 | ANKRD30B | ankyrin repeat domain 30B | 180777 | 183206 | 278923 | 40, 23, 38 |
| 18 | ANKRD62 | ankyrin repeat domain 62 | 181626 | 141401 | 176194 | |
| 18 | APCDD1 | APC down-regulated 1 | 154856 | 101558 | 134265 | 34, 37, 15 |
| 18 | AQP4 | aquaporin 4 | 171885 | 134504 | 154080 | 8, 26, 31 |
| 18 | NARS | asparaginyl-tRNA synthetase | 134440 | 066926 | 081923 | 36, 39, 3 |
| 18 | ATP5A1 | ATP synthase, H+ transporting, mitochondrial | 152234 | 152229 | 152240 | 14, 15, 43 |
| | | F1 complex, alpha subunit 1, cardiac muscle | | | | |
| 18 | ATP8B1 | ATPase phospholipid transporting 8B1 | 081923 | 134440 | 049759 | 4, 37, 42 |
| 18 | ATP9B | ATPase phospholipid transporting 9B (putative) | 166377 | 256463 | 131196 | 33, 23, 26 |
| 18 | BCL2 | BCL2, apoptosis regulator | 171791 | 081913 | 119537 | 21, 29, 37 |
| 18 | B4GALT6 | beta-1,4-galactosyltransferase 6 | 118276 | 118271 | 141437 | 31, 7, 39 |
| 18 | BOD1L2 | biorientation of chromosomes in cell division 1 like 2 | 228075 | 091157 | 177511 | 38, 8, |
| 18 | CDH19 | cadherin 19 | 071991 | 081138 | 171451 | 26, 42, 35 |
| 18 | CDH2 | cadherin 2 | 170558 | 154080 | 134762 | 45, 12, 38 |
| 18 | CDH20 | cadherin 20 | 101542 | 166603 | 176641 | 45, 8, 14 |
| 18 | CDH7 | cadherin 7 | 081138 | 166401 | 071991 | 8, 12, |
| 18 | CABYR | calcium binding tyrosine phosphorylation regulated | 154040 | 168234 | 141447 | 38, 13, 15 |
| 18 | CTIF | cap binding complex dependent translation initiation factor | 134030 | 184828 | 101665 | 1, 26, 14 |
| 18 | CHST9 | carbohydrate sulfotransferase 9 | 154080 | 171885 | 170558 | |
| 18 | CNDP1 | carnosine dipeptidase 1 | 150656 | 133313 | 215421 | 8, 10, 17 |
| 18 | CD226 | CD226 molecule | 150637 | 206052 | 176225 | 36, 5, 45 |
| 18 | CABLES1 | Cdk5 and Abl enzyme substrate 1 | 134508 | 101773 | 134490 | 18, 29, 43 |
| 18 | CIDEA | cell death-inducing DFFA-like effector a | 176194 | 181626 | 176014 | 1, 35, 26 |
| 18 | CETN1 | centrin 1 | 177143 | 158270 | 079101 | 38, 32, 8 |
| 18 | CEP192 | centrosomal protein 192 | 101639 | 085415 | 168675 | 22, 40, 1 |
| 18 | CEP76 | centrosomal protein 76 | 101624 | 128789 | 175354 | 24, 38, 30 |
| 18 | CBLN2 | cerebellin 2 precursor | 141668 | 170677 | 166342 | |
| 18 | CHMP1B | charged multivesicular body protein 1B | 255112 | 141404 | 154889 | 11, 43, 26 |
| 18 | C18orf21 | chromosome 18 open reading frame 21 | 141428 | 141429 | 141425 | 24, |
| 18 | C18orf25 | chromosome 18 open reading frame 25 | 152242 | 152240 | 141622 | 26, 13, 45 |
| 18 | C18orf32 | chromosome 18 open reading frame 32 | 177576 | 141627 | 215472 | 4, 34, 31 |
| 18 | C18orf54 | chromosome 18 open reading frame 54 | 166845 | 174448 | 178690 | 22, 12, 38 |
| 18 | C18orf63 | chromosome 18 open reading frame 63 | 206043 | 166347 | 187773 | 38, |
| 18 | C18orf8 | chromosome 18 open reading frame 8 | 141452 | 101782 | 141458 | 34, 5, 40 |
| 18 | CFAP53 | cilia and flagella associated protein 53 | 172361 | 167306 | 141644 | 38, 17, 20 |
| 18 | CLUL1 | clusterin like 1 | 079101 | 177143 | 176890 | 31, 25, 14 |
| 18 | CNDP2 | CNDP dipeptidase 2 (metallopeptidase M20 family) | 133313 | 187773 | 150656 | 36, 17, 21 |
| 18 | CCDC102B | coiled-coil domain containing 102B | 150636 | 166479 | 206052 | 24, 38, 10 |
| 18 | CCDC178 | coiled-coil domain containing 178 | 166960 | 197705 | 141431 | 10, 38, 8 |
| 18 | CCDC68 | coiled-coil domain containing 68 | 166510 | 041353 | 196628 | 31, 42, 3 |
| 18 | CCBE1 | collagen and calcium binding EGF domains 1 | 183287 | 074695 | 141682 | 26, 27, 19 |
| 18 | COLEC12 | collectin subfamily member 12 | 158270 | 079134 | 177143 | 11, 32, 6 |
| 18 | CPLX4 | complexin 4 | 166569 | 134438 | 074695 | 14, 25, 8 |
| 18 | CTDP1 | CTD phosphatase subunit 1 | 060069 | 131196 | 178342 | 38, |
| 18 | CELF4 | CUGBP, Elav-like family member 4 | 101489 | 150477 | 078142 | 31, 8, 20 |
| 18 | CTAGE1 | cutaneous T-cell lymphoma-associated antigen 1 | 212710 | 141448 | 101773 | 38, 17, 32 |
| 18 | CXXC1 | CXXC finger protein 1 | 154832 | 141644 | 154839 | 18, 26, 21 |
| 18 | CYB5A | cytochrome b5 type A | 166347 | 075336 | 206043 | 10, 29, 33 |
| 18 | DCC | DCC netrin 1 receptor | 187323 | 176624 | 280098 | |
| 18 | DSEL | dermatan sulfate epimerase-like | 171451 | 071991 | 166479 | 43, 45, 15 |
| 18 | DSC1 | desmocollin 1 | 134765 | 134755 | 134760 | 35, 15, 12 |
| 18 | DSC2 | desmocollin 2 | 134755 | 134762 | 134765 | 18, 24, 32 |
| 18 | DSC3 | desmocollin 3 | 134762 | 170558 | 134755 | 10, 24, 42 |
| 18 | DSG1 | desmoglein 1 | 134760 | 134765 | 175065 | 35, 15, 14 |
| 18 | DSG2 | desmoglein 2 | 046604 | 134757 | 118271 | 34, 13, 24 |
| 18 | DSG3 | desmoglein 3 | 134757 | 175065 | 046604 | 30, 24, 13 |
| 18 | DSG4 | desmoglein 4 | 175065 | 134760 | 134757 | 33, 38, |
| 18 | DLGAP1 | DLG associated protein 1 | 170579 | 177426 | 231824 | |
| 18 | POLI | DNA polymerase iota | 101751 | 134046 | 174448 | 29, 12, 32 |
| 18 | DOK6 | docking protein 6 | 206052 | 150636 | 150637 | 11, 29, 32 |
| 18 | DYM | dymeclin | 141627 | 101665 | 177576 | 38, |
| 18 | DYNAP | dynactin associated protein | 178690 | 166845 | 041353 | 24, 32, |
| 18 | DTNA | dystrobrevin alpha | 134769 | 101746 | 166974 | 8, 15, 26 |
| 18 | EPG5 | ectopic P-granules autophagy protein 5 homolog | 152223 | 197046 | 152229 | 26, 18, 22 |
| 18 | ELAC1 | elaC ribonuclease Z 1 | 141642 | 082212 | 267699 | 11, 25, 22 |
| 18 | EMILIN2 | elastin microfibril interfacer 2 | 132205 | 101596 | 101577 | 40, 33, 18 |
| 18 | ELP2 | elongator acetyltransferase complex subunit 2 | 134759 | 141424 | 075643 | 1, 39, 23 |
| 18 | ENOSF1 | enolase superfamily member 1 | 132199 | 176890 | 176105 | 31, 19, 11 |
| 18 | EPB41L3 | erythrocyte membrane protein band 4.1 like 3 | 082397 | 198081 | 206432 | |
| 18 | ESCO1 | establishment of sister chromatid cohesion | 141446 | 141449 | 167088 | 26, 31, 34 |
| | | N-acetyltransferase 1 | | | | |
| 18 | FAM210A | family with sequence similarity 210 member A | 177150 | 168675 | 101654 | 29, 4, 14 |
| 18 | FAM69C | family with sequence similarity 69 member C | 187773 | 206043 | 133313 | 26, 15, 14 |
| 18 | FBXO15 | F-box protein 15 | 141665 | 166342 | 075336 | 10, 38, 6 |
| 18 | FECH | ferrochelatase | 066926 | 119547 | 134440 | 43, 33, 11 |
| 18 | FHOD3 | formin homology 2 domain containing 3 | 134775 | 075643 | 134779 | 26, 42, 15 |
| 18 | GNAL | G protein subunit alpha L | 141404 | 236396 | 255112 | 8, 11, 31 |
| 18 | GALR1 | galanin receptor 1 | 166573 | 197971 | 256463 | 26, 33, |
| 18 | GRP | gastrin releasing peptide | 134443 | 166562 | 134438 | 29, 20, 44 |
| 18 | GATA6 | GATA binding protein 6 | 141448 | 101752 | 212710 | 27, 1, 9 |
| 18 | GAREM1 | GRB2 associated regulator of MAPK1 subtype 1 | 141441 | 134758 | 141434 | |
| 18 | GREB1L | growth regulation by estrogen in breast cancer 1 like | 141449 | 067900 | 141446 | 12, 27, 14 |
| 18 | HDHD2 | haloacid dehalogenase like hydrolase domain containing 2 | 167220 | 206181 | 267228 | 26, 1, 36 |
| 18 | HAUS1 | HAUS augmin like complex subunit 1 | 152240 | 152234 | 152242 | 14, 29, 26 |
| 18 | HSBP1L1 | heat shock factor binding protein 1 like 1 | 226742 | 122490 | 141759 | 28, 44, 37 |
| 18 | HRH4 | histamine receptor H4 | 134489 | 154059 | 198795 | 38, |
| 18 | HMSD | histocompatibility minor serpin domain containing | 221887 | 242550 | 166401 | 14, 8, |
| 18 | IER3IP1 | immediate early response 3 interacting protein 1 | 134049 | 267228 | 215474 | 34, 7, 4 |
| 18 | IMPACT | impact RWD domain protein | 154059 | 141447 | 134489 | 29, 4, 1 |
| 18 | INO80C | INO80 complex subunit C | 153391 | 267140 | 141429 | 3, 33, 13 |
| 18 | IMPA2 | inositol monophosphatase 2 | 141401 | 154889 | 181626 | 34, 32, 9 |
| 18 | KATNAL2 | katanin catalytic subunit A1 like 2 | 167216 | 078043 | 274744 | 38, 8, 22 |
| 18 | KLHL14 | kelch like family member 14 | 197705 | 141434 | 166960 | 29, 22, 32 |
| 18 | KIAA1328 | KIAA1328 | 150477 | 134779 | 101489 | |
| 18 | KIAA1468 | KIAA1468 | 134444 | 197563 | 141655 | 26, 11, 7 |
| 18 | L3MBTL4 | I(3)mbt-like 4 (Drosophila) | 154655 | 206432 | 088756 | 38, 15, 14 |
| 18 | LAMA1 | laminin subunit alpha 1 | 101680 | 088756 | 206422 | 40, 12, 38 |
| 18 | LAMA3 | laminin subunit alpha 3 | 053747 | 154065 | 168234 | |
| 18 | LMAN1 | lectin, mannose binding 1 | 074695 | 166569 | 183287 | 13, 42, 11 |
| 18 | LRRC30 | leucine rich repeat containing 30 | 206422 | 101680 | 173482 | |
| 18 | LIPG | lipase G, endothelial type | 101670 | 265681 | 167315 | 40, 32, 25 |
| 18 | LPIN2 | lipin 2 | 101577 | 132205 | 101605 | 19, 26, 15 |
| 18 | LOXHD1 | lipoxygenase homology domains 1 | 167210 | 141622 | 101638 | 38, 5, 33 |
| 18 | LDLRAD4 | low density lipoprotein receptor class A domain containing 4 | 168675 | 101639 | 177150 | 39, 26, 8 |
| 18 | MRO | maestro | 134042 | 141639 | 082212 | 17, 8, 38 |
| 18 | ME2 | malic enzyme 2 | 082212 | 134042 | 141642 | 43, 31, 2 |
| 18 | MALT1 | MALT1 paracaspase | 172175 | 198796 | 074657 | 22, 41, 5 |
| 18 | MC2R | melanocortin 2 receptor | 185231 | 176136 | 175322 | 2, 8, |
| 18 | MC4R | melanocortin 4 receptor | 166603 | 141682 | 101542 | |
| 18 | MC5R | melanocortin 5 receptor | 176136 | 101654 | 185231 | |
| 18 | MEP1B | meprin A subunit beta | 141434 | 141441 | 197705 | 16, 21, 40 |
| 18 | MPPE1 | metallophosphoesterase 1 | 154889 | 255112 | 141401 | 22, 40, 26 |
| 18 | MBD1 | methyl-CpG binding domain protein 1 | 141644 | 172361 | 154832 | 38, 14, 6 |
| 18 | MBD2 | methyl-CpG binding domain protein 2 | 134046 | 280098 | 101751 | 10, 18, 4 |
| 18 | METTL4 | methyltransferase like 4 | 101574 | 141433 | 080986 | 1, 23, 39 |
| 18 | MEX3C | mex-3 RNA binding family member C | 176624 | 141646 | 187323 | 40, 29, 22 |
| 18 | MAPRE2 | microtubule associated protein RP/EB family member 2 | 166974 | 134769 | 186812 | |
| 18 | MTCL1 | microtubule crosslinking factor 1 | 168502 | 206418 | 178127 | 11, 31, 14 |
| 18 | MIB1 | mindbomb E3 ubiquitin protein ligase 1 | 101752 | 158201 | 141448 | 22, 29, 11 |
| 18 | MAPK4 | mitogen-activated protein kinase 4 | 141639 | 154839 | 134042 | 29, 11, 14 |
| 18 | MOCOS | molybdenum cofactor sulfurase | 075643 | 134759 | 134775 | 9, 34, 13 |
| 18 | MBP | myelin basic protein | 197971 | 130856 | 166573 | 8, 17, 26 |
| 18 | MYOM1 | myomesin 1 | 101605 | 101577 | 101608 | 15, 25, 18 |
| 18 | MYL12A | myosin light chain 12A | 101608 | 101605 | 118680 | |
| 18 | MYL12B | myosin light chain 12B | 118680 | 101608 | 177426 | 4, 43, 30 |
| 18 | MYO5B | myosin VB | 167306 | 266997 | 172361 | 3, 23, 40 |
| 18 | NDUFV2 | NADH: ubiquinone oxidoreductase core subunit V2 | 178127 | 168502 | 101745 | 10, 30, 33 |
| 18 | NDC80 | NDC80, kinetochore complex component | 080986 | 101574 | 101596 | 4, 9, 39 |
| 18 | NEDD4L | neural precursor cell expressed, developmentally | 049759 | 081923 | 198796 | 33, 35, 19 |
| | | down-regulated 4-like, E3 ubiquitin protein ligase | | | | |
| 18 | NETO1 | neuropilin and tolloid like 1 | 166342 | 141668 | 141665 | 33, 14, 4 |
| 18 | NPC1 | NPC intracellular cholesterol transporter 1 | 141458 | 141452 | 154065 | 2, 42, 6 |
| 18 | NAPG | NSF attachment protein gamma | 134265 | 154856 | 154864 | 34, 29, 26 |
| 18 | NFATC1 | nuclear factor of activated T-cells 1 | 131196 | 166377 | 060069 | 22, 29, 15 |
| 18 | NOL4 | nucleolar protein 4 | 101746 | 141431 | 134769 | 8, 20, 28 |
| 18 | ONECUT2 | one cut homeobox 2 | 119547 | 177511 | 066926 | 26, 20, 14 |
| 18 | OSBPL1A | oxysterol binding protein like 1A | 141447 | 154040 | 154059 | |
| 18 | PARD6G | par-6 family cell polarity regulator gamma | 178184 | 101544 | 166562 | 27, 29, 35 |
| 18 | PHLPP1 | PH domain and leucine rich repeat protein phosphatase 1 | 081913 | 141664 | 171791 | 14, 26, 11 |
| 18 | PMAIP1 | phorbol-12-myristate-13-acetate-induced protein 1 | 141682 | 183287 | 166603 | 4, 7, 34 |
| 18 | PIK3C3 | phosphatidylinositol 3-kinase catalytic subunit type 3 | 078142 | 101489 | 152214 | 43, 29, 6 |
| 18 | PIGN | phosphatidylinositol glycan anchor biosynthesis class N | 197563 | 176641 | 134444 | 42, 4, 11 |
| 18 | PIEZO2 | piezo type mechanosensitive ion channel component 2 | 154864 | 134265 | 236396 | 45, 20, 15 |
| 18 | GALNT1 | polypeptide N-acetylgalactosaminyltransferase 1 | 141429 | 153391 | 141428 | |
| 18 | KCTD1 | potassium channel tetramerization domain containing 1 | 134504 | 141384 | 171885 | 15, 32, 29 |
| 18 | KCNG2 | potassium voltage-gated channel modifier subfamily G member 2 | 178342 | 060069 | 122490 | |
| 18 | POTEC | POTE ankyrin domain family member C | 183206 | 175322 | 180777 | 25, 38, |
| 18 | PQLC1 | PQ loop repeat containing 1 | 122490 | 178342 | 226742 | 26, 1, 29 |
| 18 | PRELID3A | PRELI domain containing 3A | 141391 | 141385 | 134278 | 13, 37, 35 |
| 18 | PSTPIP2 | proline-serine-threonine phosphatase interacting protein 2 | 152229 | 152223 | 152234 | 43, 36, 5 |
| 18 | PSMG2 | proteasome assembly chaperone 2 | 128789 | 134278 | 101624 | 4, 40, 38 |
| 18 | PSMA8 | proteasome subunit alpha 8 | 154611 | 141380 | 141384 | 38, 30, 10 |
| 18 | PIAS2 | protein inhibitor of activated STAT 2 | 078043 | 101638 | 167216 | 38, |
| 18 | PPP4R1 | protein phosphatase 4 regulatory subunit 1 | 154845 | 017797 | 168461 | 29, 42, 23 |
| 18 | PTPN2 | protein tyrosine phosphatase, non-receptor type 2 | 175354 | 101624 | 085415 | 6, 15, 4 |
| 18 | PTPRM | protein tyrosine phosphatase, receptor type M | 173482 | 206422 | 206418 | 40, 39, 6 |
| 18 | RAB12 | RAB12, member RAS oncogene family | 206418 | 173482 | 168502 | 13, 23, 18 |
| 18 | RAB27B | RAB27B, member RAS oncogene family | 041353 | 178690 | 166510 | 42, 26, 25 |
| 18 | RAB31 | RAB31, member RAS oncogene family | 168461 | 154845 | 168454 | 6, 9, 1 |
| 18 | RALBP1 | ralA binding protein 1 | 017797 | 128791 | 154845 | 24, 31, 6 |
| 18 | RIT2 | Ras like without CAAX 2 | 152214 | 078142 | 132872 | 10, 8, 14 |
| 18 | RBBP8 | RB binding protein 8, endonuclease | 101773 | 212710 | 134508 | |
| 18 | RPRD1A | regulation of nuclear pre-mRNA domain containing 1A | 141425 | 141428 | 141424 | |
| 18 | RAX | retina and anterior neural fold homeobox | 134438 | 134443 | 166569 | |
| 18 | ROCK1 | Rho associated coiled-coil containing protein kinase 1 | 067900 | 278923 | 141449 | 22, 26, 11 |
| 18 | ARHGAP28 | Rho GTPase activating protein 28 | 088756 | 154655 | 101680 | |
| 18 | RPL17 | ribosomal protein L17 | 265681 | 215472 | 101670 | |
| 18 | RBFA | ribosome binding factor A (putative) | 101546 | 267127 | 101544 | 18, 1, 5 |
| 18 | RNF125 | ring finger protein 125 | 101695 | 153339 | 134758 | 35, 10, 9 |
| 18 | RNF138 | ring finger protein 138 | 134758 | 101695 | 141441 | 38, 13, 42 |
| 18 | RNF152 | ring finger protein 152 | 176641 | 101542 | 197563 | 18, 1, 26 |
| 18 | RNF165 | ring finger protein 165 | 141622 | 152242 | 167210 | 5, 8, 14 |
| 18 | RIOK3 | RIO kinase 3 | 101782 | 134490 | 141452 | 24, 42, 39 |
| 18 | RNMT | RNA guanine-7 methyltransferase | 101654 | 177150 | 176136 | 24, 40, 3 |
| 18 | RTTN | rotatin | 176225 | 150637 | 170677 | 40, 26, 23 |
| 18 | RPL17-C18orf32 | RPL17-C18orf32 readthrough | 215472 | 177576 | 265681 | |
| 18 | SEC11C | SEC11 homolog C, signal peptidase complex subunit | 166562 | 074657 | 134443 | |
| 18 | SEH1L | SEH1 like nucleoporin | 085415 | 175354 | 101639 | 11, 42, 6 |
| 18 | SERPINB10 | serpin family B member 10 | 242550 | 197632 | 221887 | 7, 5, 24 |
| 18 | SERPINB11 | serpin family B member 11 (gene/pseudogene) | 206072 | 206073 | 057149 | 42, 13, 33 |
| 18 | SERPINB12 | serpin family B member 12 | 166634 | 206075 | 197641 | 35, 24, |
| 18 | SERPINB13 | serpin family B member 13 | 197641 | 166634 | 206073 | 24, 42, 13 |
| 18 | SERPINB2 | serpin family B member 2 | 197632 | 166396 | 242550 | 5, 13, 45 |
| 18 | SERPINB3 | serpin family B member 3 | 057149 | 206072 | 166396 | 18, 30, 24 |
| 18 | SERPINB4 | serpin family B member 4 | 206073 | 197641 | 206072 | 13, 42, 30 |
| 18 | SERPINB5 | serpin family B member 5 | 206075 | 119541 | 166634 | 24, 13, 18 |
| 18 | SERPINB7 | serpin family B member 7 | 166396 | 057149 | 197632 | 43, 35, 13 |
| 18 | SERPINB8 | serpin family B member 8 | 166401 | 221887 | 081138 | 45, 35, 34 |
| 18 | SETBP1 | SET binding protein 1 | 152217 | 132872 | 132874 | 11, 22, 24 |
| 18 | SIGLEC15 | sialic acid binding Ig like lectin 15 | 197046 | 141469 | 152223 | 5, 6, 36 |
| 18 | SKOR2 | SKI family transcriptional corepressor 2 | 215474 | 134049 | 175387 | |
| 18 | SMAD2 | SMAD family member 2 | 175387 | 215474 | 184828 | 32, 27, |
| 18 | SMAD4 | SMAD family member 4 | 141646 | 267699 | 176624 | 11, 40, 29 |
| 18 | SMAD7 | SMAD family member 7 | 101665 | 134030 | 141627 | 26, 15, 45 |
| 18 | SMIM21 | small integral membrane protein 21 | 206026 | 179981 | 101493 | 38, |
| 18 | SNRPD1 | small nuclear ribonucleoprotein D1 polypeptide | 167088 | 141446 | 158201 | 30, 12, 7 |
| 18 | SLC14A1 | solute carrier family 14 member 1 (Kidd blood group) | 141469 | 132874 | 197046 | 33, 26, 4 |
| 18 | SLC14A2 | solute carrier family 14 member 2 | 132874 | 152217 | 141469 | 16, 38, 5 |
| 18 | SLC25A52 | solute carrier family 25 member 52 | 141437 | 118276 | 153339 | 38, |
| 18 | SLC35G4 | solute carrier family 35 member G4 | 236396 | 154864 | 141404 | |
| 18 | SLC39A6 | solute carrier family 39 member 6 | 141424 | 141425 | 134759 | 11, 40, 18 |
| 18 | SALL3 | spalt like transcription factor 3 | 256463 | 166573 | 166377 | 12, 17, 14 |
| 18 | SKA1 | spindle and kinetochore associated complex subunit 1 | 154839 | 154832 | 141639 | 41, 34, 2 |
| 18 | SPIRE1 | spire type actin nucleation factor 1 | 134278 | 141391 | 128789 | 11, 29, 20 |
| 18 | SS18 | SS18, nBAF chromatin remodeling complex subunit | 141380 | 198795 | 154611 | 42, 24, 39 |
| 18 | ST8SIA3 | ST8 alpha-N-acetyl-neuraminide alpha-2,8-sialyltransferase 3 | 177511 | 228075 | 119547 | 31, 8, 20 |
| 18 | ST8SIA5 | ST8 alpha-N-acetyl-neuraminide alpha-2,8-sialyltransferase 5 | 101638 | 167210 | 078043 | |
| 18 | STARD6 | StAR related lipid transfer domain containing 6 | 174448 | 101751 | 166845 | 38, |
| 18 | SMCHD1 | structural maintenance of chromosomes flexible hinge | 101596 | 080986 | 132205 | 22, 1, 37 |
| | | domain containing 1 | | | | |
| 18 | SOCS6 | suppressor of cytokine signaling 6 | 170677 | 176225 | 141668 | 34, 29, 44 |
| 18 | SYT4 | synaptotagmin 4 | 132872 | 152214 | 152217 | 8, 26, 31 |
| 18 | TAF4B | TATA-box binding protein associated factor 4b | 141384 | 154611 | 134504 | 22, 9, 45 |
| 18 | TSHZ1 | teashirt zinc finger homeobox 1 | 179981 | 180011 | 206026 | 1, 26, 29 |
| 18 | TTC39C | tetratricopeptide repeat domain 39C | 168234 | 053747 | 154040 | 22, 5, |
| 18 | TGIF1 | TGFB induced factor homeobox 1 | 177426 | 118680 | 170579 | |
| 18 | TXNDC2 | thioredoxin domain containing 2 | 168454 | 168461 | 101558 | 38, 8, 32 |
| 18 | TXNL1 | thioredoxin like 1 | 091164 | 196628 | 091157 | |
| 18 | TXNL4A | thioredoxin like 4A | 141759 | 226742 | 267127 | 30, 41, 21 |
| 18 | TMX3 | thioredoxin related transmembrane protein 3 | 166479 | 171451 | 150636 | 11, 42, 26 |
| 18 | THOC1 | THO complex 1 | 079134 | 101557 | 158270 | 18, 10, 22 |
| 18 | TYMS | thymidylate synthetase | 176890 | 079101 | 132199 | 21, 9, 4 |
| 18 | TNFRSF11A | TNF receptor superfamily member 11a | 141655 | 134444 | 141664 | |
| 18 | TRAPPC8 | trafficking protein particle complex 8 | 153339 | 141437 | 101695 | 26, 4, 9 |
| 18 | TCEB3B | Transcription elongation factor B subunit 3B | 206181 | 183791 | 167220 | 38, |
| 18 | TCEB3C | Transcription elongation factor B subunit 3C | 183791 | 278674 | 206181 | |
| 18 | TCEB3CL | Transcription elongation factor B subunit 3C like | 275553 | 274744 | 278674 | |
| 18 | TCEB3CL | Transcription elongation factor B subunit 3C like | 278674 | 275553 | 183791 | |
| 18 | TCEB3CL2 | Transcription elongation factor B subunit 3C like 2 | 274744 | 167216 | 275553 | |
| 18 | TCF4 | transcription factor 4 | 196628 | 166510 | 091164 | 45, 30, 43 |
| 18 | TIMM21 | translocase of inner mitochondrial membrane 21 | 075336 | 141665 | 166347 | 1, 30, 40 |
| 18 | TMEM200C | transmembrane protein 200C | 206432 | 082397 | 154655 | 23, 12, 15 |
| 18 | TMEM241 | transmembrane protein 241 | 134490 | 134508 | 101782 | 33, 44, 32 |
| 18 | TTR | transthyretin | 118271 | 046604 | 118276 | 28, 29, 19 |
| 18 | TUBB6 | tubulin beta 6 class V | 176014 | 176194 | 141385 | |
| 18 | TPGS2 | tubulin polyglutamylase complex subunit 2 | 134779 | 134775 | 150477 | 38, 14, 9 |
| 18 | TWSG1 | twisted gastrulation BMP signaling modulator 1 | 128791 | 101745 | 017797 | 11, 42, 4 |
| 18 | USP14 | ubiquitin specific peptidase 14 | 101557 | 166562 | 079134 | 4, 19, 13 |
| 18 | VPS4B | vacuolar protein sorting 4 homolog B | 119541 | 119537 | 206075 | 30, 13, 38 |
| 18 | VAPA | VAMP associated protein A | 101558 | 168454 | 154856 | 31, 26, 10 |
| 18 | WDR7 | WD repeat domain 7 | 091157 | 091164 | 228075 | 10, |
| 18 | YES1 | YES proto-oncogene 1, Src family tyrosine kinase | 176105 | 132199 | 141433 | 11, 13, 42 |
| 18 | ZADH2 | zinc binding alcohol dehydrogenase domain containing 2 | 180011 | 215421 | 179981 | 34, 22, 1 |
| 18 | ZBTB14 | zinc finger and BTB domain containing 14 | 198081 | 231824 | 082397 | 11, 29, 22 |
| 18 | ZBTB7C | zinc finger and BTB domain containing 7C | 184828 | 175387 | 134030 | 14, 32, |
| 18 | ZSCAN30 | zinc finger and SCAN domain containing 30 | 186814 | 186812 | 172466 | 29, 42, 39 |
| 18 | ZCCHC2 | zinc finger CCHC-type containing 2 | 141664 | 141655 | 081913 | |
| 18 | ZNF236 | zinc finger protein 236 | 130856 | 101493 | 197971 | 1, 40, 36 |
| 18 | ZNF24 | zinc finger protein 24 | 172466 | 186814 | 186496 | 40, 30, 13 |
| 18 | ZNF396 | zinc finger protein 396 | 186496 | 172466 | 267140 | 31, 14, 42 |
| 18 | ZNF397 | zinc finger protein 397 | 186812 | 166974 | 186814 | 43, 22, 26 |
| 18 | ZNF407 | zinc finger protein 407 | 215421 | 150656 | 180011 | 22, 1, 40 |
| 18 | ZNF516 | zinc finger protein 516 | 101493 | 206026 | 130856 | |
| 18 | ZNF519 | zinc finger protein 519 | 175322 | 185231 | 183206 | 22, 36, 8 |
| 18 | ZNF521 | zinc finger protein 521 | 198795 | 134489 | 141380 | 43, 11, 31 |
| 18 | ZNF532 | zinc finger protein 532 | 074657 | 172175 | 166562 | 18, 24, 12 |
| 19 | PGLS | 6-phosphogluconolactonase | 130313 | 130304 | 167483 | 21, 22, 27 |
| 19 | ABHD17A | abhydrolase domain containing 17A | 129968 | 129911 | 227500 | |
| 19 | ABHD8 | abhydrolase domain containing 8 | 127220 | 160117 | 130312 | 2, 39, 38 |
| 19 | ACP5 | acid phosphatase 5, tartrate resistant | 102575 | 198551 | 197933 | 43, 6, 41 |
| 19 | ACP7 | acid phosphatase 7, tartrate resistant (putative) | 183760 | 161243 | 130669 | 28, 44, 8 |
| 19 | ACPT | acid phosphatase, testicular | 142513 | 261341 | 167747 | 38, |
| 19 | ACTL9 | actin like 9 | 181786 | 142303 | 181733 | 38, 8, |
| 19 | ACTN4 | actinin alpha 4 | 130402 | 178982 | 182472 | 18, 33, 40 |
| 19 | ATF5 | activating transcription factor 5 | 169136 | 269179 | 161640 | 21, 19, 25 |
| 19 | ACSBG2 | acyl-CoA synthetase bubblegum family member 2 | 130377 | 087903 | 130382 | 38, 8, 32 |
| 19 | ADAMTS10 | ADAM metallopeptidase with thrombospondin type 1 motif 10 | 142303 | 142347 | 181786 | 15, 26, 39 |
| 19 | ADAMTSL5 | ADAMTS like 5 | 185761 | 115255 | 185988 | |
| 19 | AP1M1 | adaptor related protein complex 1 mu 1 subunit | 072958 | 105058 | 127528 | 21, 3, 35 |
| 19 | AP1M2 | adaptor related protein complex 1 mu 2 subunit | 129354 | 129355 | 129353 | 31, 13, 16 |
| 19 | AP2A1 | adaptor related protein complex 2 alpha 1 subunit | 196961 | 126467 | 010361 | 40, 21, 34 |
| 19 | AP2S1 | adaptor related protein complex 2 sigma 1 subunit | 042753 | 280050 | 160007 | 35, 15, 6 |
| 19 | AP3D1 | adaptor related protein complex 3 delta 1 subunit | 065000 | 099840 | 104885 | 35, 1, 27 |
| 19 | ADAT3 | adenosine deaminase, tRNA specific 3 | 213638 | 227500 | 133275 | |
| 19 | ADGRE1 | adhesion G protein-coupled receptor E1 | 174837 | 141968 | 196589 | 36, 39, 5 |
| 19 | ADGRE2 | adhesion G protein-coupled receptor E2 | 127507 | 160961 | 188269 | 7, 36, 5 |
| 19 | ADGRE3 | adhesion G protein-coupled receptor E3 | 131355 | 187912 | 160961 | |
| 19 | ADGRE5 | adhesion G protein-coupled receptor E5 | 123146 | 072071 | 123136 | 36, 39, 5 |
| 19 | ADGRL1 | adhesion G protein-coupled receptor L1 | 072071 | 105011 | 123146 | 14, 27, 31 |
| 19 | ADM5 | adrenomedullin 5 (putative) | 224420 | 126457 | 169169 | 9, 6, 16 |
| 19 | AKAP8 | A-kinase anchoring protein 8 | 105127 | 141867 | 011243 | 1, 43, 6 |
| 19 | AKAP8L | A-kinase anchoring protein 8 like | 011243 | 105127 | 011451 | 39, 36, 41 |
| 19 | AKT2 | AKT serine/threonine kinase 2 | 105221 | 105219 | 160392 | |
| 19 | AKT1S1 | AKT1 substrate 1 | 204673 | 039650 | 104946 | |
| 19 | ALDH16A1 | aldehyde dehydrogenase 16 family member A1 | 161618 | 104872 | 269469 | 21, 7, 34 |
| 19 | ACER1 | alkaline ceramidase 1 | 167769 | 130382 | 125656 | 35, |
| 19 | ALKBH6 | alkB homolog 6 | 239382 | 181392 | 105270 | 3, 39, 6 |
| 19 | ALKBH7 | alkB homolog 7 | 125652 | 125656 | 125650 | 29, 41, 18 |
| 19 | A1BG | alpha-1-B glycoprotein | 121410 | 182318 | 174586 | 19, 23, 5 |
| 19 | AES | amino-terminal enhancer of split | 104964 | 065717 | 088256 | 41, 26, 31 |
| 19 | APLP1 | amyloid beta precursor like protein 1 | 105290 | 126259 | 167604 | 8, 28, 31 |
| 19 | APBA3 | amyloid beta precursor protein binding family A member 3 | 011132 | 105289 | 183617 | 41, 28, 30 |
| 19 | ANGPTL4 | angiopoietin like 4 | 167772 | 186994 | 185236 | |
| 19 | ANGPTL6 | angiopoietin like 6 | 130812 | 130813 | 243207 | 32, 16, 38 |
| 19 | ANGPTL8 | angiopoietin like 8 | 130173 | 130158 | 130167 | 19, 15, 17 |
| 19 | ANKLE1 | ankyrin repeat and LEM domain containing 1 | 160117 | 269307 | 127220 | 21, 39, 5 |
| 19 | ANKRD24 | ankyrin repeat domain 24 | 089847 | 077463 | 105246 | 32, 37, 17 |
| 19 | ANKRD27 | ankyrin repeat domain 27 | 105186 | 105185 | 186326 | 22, 31, 27 |
| 19 | ANO8 | anoctamin 8 | 074855 | 130311 | 130299 | 26, 12, 36 |
| 19 | AMH | anti-Mullerian hormone | 104899 | 104897 | 167476 | 12, 44, 37 |
| 19 | ASF1B | anti-silencing function 1B histone chaperone | 105011 | 072062 | 072071 | 41, 21, 9 |
| 19 | APC2 | APC2, WNT signaling pathway regulator | 115266 | 115268 | 119559 | 26, 8, 14 |
| 19 | APOC4-APOC2 | APOC4-APOC2 readthrough (NMD candidate) | 224916 | 130208 | 234906 | 19, 17, 10 |
| 19 | APOC1 | apolipoprotein C1 | 130208 | 130203 | 224916 | 19, 2, 40 |
| 19 | APOC2 | apolipoprotein C2 | 234906 | 224916 | 104853 | 19, 36, 31 |
| 19 | APOC4 | apolipoprotein C4 | 267467 | 130208 | 234906 | 19, 17, 10 |
| 19 | APOE | apolipoprotein E | 130203 | 130204 | 130208 | 35, 2, 8 |
| 19 | ARMC6 | armadillo repeat containing 6 | 105676 | 064607 | 181035 | 9, 35, 43 |
| 19 | ARRDC2 | arrestin domain containing 2 | 105643 | 105642 | 096996 | 39, 1, 36 |
| 19 | ARRDC5 | arrestin domain containing 5 | 205784 | 105355 | 276043 | 17, 38, 23 |
| 19 | ASNA1 | arsA arsenite transporter, ATP-binding, homolog 1 (bacterial) | 198356 | 123144 | 039987 | 34, 35, 6 |
| 19 | ASPDH | aspartate dehydrogenase domain containing | 204653 | 161677 | 131409 | 17, 14, 20 |
| 19 | ATCAY | ATCAY, caytaxin | 167654 | 105278 | 077009 | 8, 26, 38 |
| 19 | ABCA7 | ATP binding cassette subfamily A member 7 | 064687 | 064666 | 180448 | 39, 22, 36 |
| 19 | ATP5D | ATP synthase, H+ transporting, mitochondrial F1 complex, | 099624 | 099625 | 167470 | 41, 25, 33 |
| | | delta subunit | | | | |
| 19 | ATP5SL | ATP5S like | 105341 | 177191 | 204978 | 33, 20, 34 |
| 19 | ATP13A1 | ATPase 13A1 | 105726 | 089639 | 181896 | 40, 39, 18 |
| 19 | ATP4A | ATPase H+/K+ transporting alpha subunit | 105675 | 105677 | 249115 | 2, 37, 8 |
| 19 | ATP1A3 | ATPase Na+/K+ transporting subunit alpha 3 | 105409 | 105404 | 105737 | 8, 14, 26 |
| 19 | ATP8B3 | ATPase phospholipid transporting 8B3 | 130270 | 205922 | 079313 | 38, 9, 10 |
| 19 | ARID3A | AT-rich interaction domain 3A | 116017 | 116014 | 065268 | 32, 12, 22 |
| 19 | AURKC | aurora kinase C | 105146 | 083844 | 204524 | 38, 26, 12 |
| 19 | ATG4D | autophagy related 4D cysteine peptidase | 130734 | 180739 | 129347 | 28, 34, 2 |
| 19 | AXL | AXL receptor tyrosine kinase | 167601 | 167600 | 105323 | 6, 15, 9 |
| 19 | AZU1 | azurocidin 1 | 172232 | 129951 | 196415 | 5, 7, 6 |
| 19 | B9D2 | B9 protein domain 2 | 123810 | 142046 | 077348 | 31, 29, 18 |
| 19 | BIRC8 | baculoviral IAP repeat containing 8 | 163098 | 228567 | 203326 | 38, 8, |
| 19 | BCAM | basal cell adhesion molecule (Lutheran blood group) | 187244 | 142273 | 130202 | 30, 27, 18 |
| 19 | BHMG1 | basic helix-loop-helix and HMG-box containing 1 | 237452 | 177051 | 177045 | |
| 19 | BSG | basigin (Ok blood group) | 172270 | 197540 | 099822 | 13, 32, 7 |
| 19 | BCL3 | B-cell CLL/lymphoma 3 | 069399 | 213892 | 142273 | 34, 27, 16 |
| 19 | BAX | BCL2 associated X, apoptosis regulator | 087088 | 104808 | 087086 | 30, 26, 24 |
| 19 | BBC3 | BCL2 binding component 3 | 105327 | 142230 | 105321 | 18, 16, 27 |
| 19 | BCL2L12 | BCL2 like 12 | 126453 | 126456 | 126457 | 39, 36, 22 |
| 19 | BEST2 | bestrophin 2 | 039987 | 198356 | 095066 | 16, 38, 12 |
| 19 | BLVRB | biliverdin reductase B | 090013 | 167565 | 160460 | 34, 29, 23 |
| 19 | BSPH1 | binder of sperm protein homolog 1 | 188334 | 105398 | 169393 | |
| 19 | BLOC1S3 | biogenesis of lysosomal organelles complex 1 subunit 3 | 189114 | 007255 | 267545 | 34, 4, 27 |
| 19 | BORCS8 | BLOC-1 related complex subunit 8 | 254901 | 213999 | 064490 | |
| 19 | BST2 | bone marrow stromal cell antigen 2 | 130303 | 269720 | 141971 | 27, 2, 1 |
| 19 | BORCS8-MEF2B | BORCS8-MEF2B readthrough | 064489 | 064545 | 213999 | |
| 19 | BRSK1 | BR serine/threonine kinase 1 | 160469 | 133265 | 180061 | 26, 15, 21 |
| 19 | BCAT2 | branched chain amino acid transaminase 2 | 105552 | 105550 | 087076 | 9, 34, 27 |
| 19 | BCKDHA | branched chain keto acid dehydrogenase E1, alpha polypeptide | 248098 | 077348 | 177191 | 1, 15, 2 |
| 19 | BABAM1 | BRISC and BRCA1A complex member 1 | 105393 | 130307 | 269307 | 25, 35, 1 |
| 19 | BRD4 | bromodomain containing 4 | 141867 | 105131 | 105127 | 22, 34, 21 |
| 19 | BTBD2 | BTB domain containing 2 | 133243 | 133275 | 099875 | |
| 19 | C2CD4C | C2 calcium dependent domain containing 4C | 183186 | 105549 | 129946 | 5, 14, 12 |
| 19 | CPAMD8 | C3 and PZP like, alpha-2-macroglobulin domain containing 8 | 160111 | 127533 | 131351 | 14, 9, 10 |
| 19 | CBARP | CACN beta subunit associated regulatory protein | 099625 | 118046 | 099624 | |
| 19 | CACTIN | cactin, spliceosome C complex subunit | 105298 | 006638 | 186111 | |
| 19 | CIB3 | calcium and integrin binding family member 3 | 141977 | 196684 | 105058 | 10, |
| 19 | CABP5 | calcium binding protein 5 | 105507 | 169393 | 105499 | 14, 25, |
| 19 | CHERP | calcium homeostasis endoplasmic reticulum protein | 085872 | 105072 | 127526 | 22, 34, 12 |
| 19 | CACNG6 | calcium voltage-gated channel auxiliary subunit gamma 6 | 130433 | 142408 | 189068 | 14, 12, 20 |
| 19 | CACNG7 | calcium voltage-gated channel auxiliary subunit gamma 7 | 105605 | 126583 | 142408 | 26, 12, 8 |
| 19 | CACNG8 | calcium voltage-gated channel auxiliary subunit gamma 8 | 142408 | 105605 | 130433 | 45, 8, 16 |
| 19 | CACNA1A | calcium voltage-gated channel subunit alpha1 A | 141837 | 160888 | 104957 | 41, 14, 27 |
| 19 | CAPS | calcyphosine | 105519 | 187650 | 031823 | 44, 26, 35 |
| 19 | CALM3 | calmodulin 3 | 160014 | 204851 | 160013 | 8, 26, 4 |
| 19 | CAMSAP3 | calmodulin regulated spectrin associated protein family | 076826 | 032444 | 076924 | 17, 16, 14 |
| | | member 3 | | | | |
| 19 | CAPN12 | calpain 12 | 182472 | 130402 | 205076 | 13, 37, 30 |
| 19 | CAPNS1 | calpain small subunit 1 | 126247 | 105254 | 161281 | 18, 34, 26 |
| 19 | CNN1 | calponin 1 | 130176 | 130159 | 130165 | |
| 19 | CNN2 | calponin 2 | 064666 | 182087 | 064687 | 5, 36, 34 |
| 19 | CALR | calreticulin | 179218 | 179115 | 179262 | 40, 23, 27 |
| 19 | CALR3 | calreticulin 3 | 269058 | 127527 | 141979 | 38, 8, |
| 19 | CREB3L3 | cAMP responsive element binding protein 3 like 3 | 060566 | 126934 | 077463 | 37, 19, 16 |
| 19 | CLIP3 | CAP-Gly domain containing linker protein 3 | 105270 | 239382 | 161277 | 8, 26, 18 |
| 19 | CIC | capicua transcriptional repressor | 079432 | 105722 | 079462 | 34, 26, 22 |
| 19 | CHST8 | carbohydrate sulfotransferase 8 | 124302 | 124299 | 153885 | 31, 28, 25 |
| 19 | CA11 | carbonic anhydrase 11 | 063180 | 105516 | 142233 | 26, 31, 8 |
| 19 | CEACAM1 | carcinoembryonic antigen related cell adhesion molecule 1 | 079385 | 189377 | 124469 | 16, 5, 30 |
| 19 | CEACAM16 | carcinoembryonic antigen related cell adhesion molecule 16 | 213892 | 186567 | 069399 | |
| 19 | CEACAM18 | carcinoembryonic antigen related cell adhesion molecule 18 | 213822 | 105366 | 254521 | |
| 19 | CEACAM19 | carcinoembryonic antigen related cell adhesion molecule 19 | 186567 | 073008 | 213892 | 26, 33, 4 |
| 19 | CEACAM20 | carcinoembryonic antigen related cell adhesion molecule 20 | 273777 | 167384 | 216588 | |
| 19 | CEACAM21 | carcinoembryonic antigen related cell adhesion molecule 21 | 007129 | 204978 | 105352 | 30, 36, 7 |
| 19 | CEACAM3 | carcinoembryonic antigen related cell adhesion molecule 3 | 170956 | 086548 | 273111 | 5, 32, 36 |
| 19 | CEACAM4 | carcinoembryonic antigen related cell adhesion molecule 4 | 105352 | 007129 | 007306 | 5, 14, 17 |
| 19 | CEACAM5 | carcinoembryonic antigen related cell adhesion molecule 5 | 105388 | 007306 | 267881 | 16, 42, 24 |
| 19 | CEACAM6 | carcinoembryonic antigen related cell adhesion molecule 6 | 086548 | 267881 | 170956 | 16, 3, 24 |
| 19 | CEACAM7 | carcinoembryonic antigen related cell adhesion molecule 7 | 007306 | 105352 | 105388 | 16, 13, 30 |
| 19 | CEACAM8 | carcinoembryonic antigen related cell adhesion molecule 8 | 124469 | 079385 | 221826 | 5, 7, 36 |
| 19 | CPT1C | carnitine palmitoyltransferase 1C | 169169 | 224420 | 126467 | 26, 8, 14 |
| 19 | CILP2 | cartilage intermediate layer protein 2 | 160161 | 250067 | 105717 | 42, 38, 10 |
| 19 | COMP | cartilage oligomeric matrix protein | 105664 | 105662 | 005007 | 10, 6, 24 |
| 19 | CSNK1G2 | casein kinase 1 gamma 2 | 133275 | 213638 | 133243 | 26, 3, 34 |
| 19 | CLPP | caseinolytic mitochondrial matrix peptidase proteolytic | 125656 | 167769 | 125652 | 21, 6, 27 |
| | | subunit | | | | |
| 19 | CASP14 | caspase 14 | 105141 | 160994 | 094661 | 35, 44, 4 |
| 19 | CARD8 | caspase recruitment domain family member 8 | 105483 | 178150 | 105479 | |
| 19 | CATSPERD | cation channel sperm associated auxiliary subunit delta | 174898 | 196365 | 212123 | 38, 2, 25 |
| 19 | CATSPERG | cation channel sperm associated auxiliary subunit gamma | 099338 | 099337 | 099341 | 38, 2, 40 |
| 19 | CBLC | Cbl proto-oncogene C | 142273 | 069399 | 187244 | 18, 37, 20 |
| 19 | CCL25 | C-C motif chemokine ligand 25 | 131142 | 066044 | 142449 | 39, 20, 37 |
| 19 | CEBPA | CCAAT/enhancer binding protein alpha | 245848 | 130876 | 153879 | 37, 1, 16 |
| 19 | CEBPG | CCAAT/enhancer binding protein gamma | 153879 | 245848 | 124299 | 34, 26, 9 |
| 19 | CNOT3 | CCR4-NOT transcription complex subunit 3 | 088038 | 105618 | 105617 | 18, 27, 31 |
| 19 | CD177 | CD177 molecule | 204936 | 183668 | 131126 | 16, 33, 37 |
| 19 | CD209 | CD209 molecule | 090659 | 182566 | 104938 | 1, 42, 25 |
| 19 | CD22 | CD22 molecule | 012124 | 105695 | 126266 | |
| 19 | CD320 | CD320 molecule | 167775 | 090661 | 167774 | 35, 21, 27 |
| 19 | CD33 | CD33 molecule | 105383 | 168995 | 179213 | 5, 29, 7 |
| 19 | CD37 | CD37 molecule | 104894 | 063127 | 074219 | 41, 36, 21 |
| 19 | CD3EAP | CD3e molecule associated protein | 117877 | 104881 | 012061 | 13, 34, 33 |
| 19 | CD70 | CD70 molecule | 125726 | 125657 | 125735 | 17, 16, |
| 19 | CD79A | CD79a molecule | 105369 | 105372 | 076928 | 41, 21, 22 |
| 19 | CDC42EP5 | CDC42 effector protein 5 | 167617 | 275183 | 167618 | 37, 3, 16 |
| 19 | CADM4 | cell adhesion molecule 4 | 105767 | 131116 | 011422 | 26, 17, 14 |
| 19 | CDC34 | cell division cycle 34 | 099804 | 141933 | 197540 | 14, 27, 35 |
| 19 | CDC37 | cell division cycle 37 | 105401 | 105397 | 065989 | 35, 26, 32 |
| 19 | CEP89 | centrosomal protein 89 | 121289 | 021488 | 131944 | 18, 31, 6 |
| 19 | CERS1 | ceramide synthase 1 | 223802 | 005007 | 105669 | 26, 8, 28 |
| 19 | CERS4 | ceramide synthase 4 | 090661 | 142449 | 167775 | 33, 41, 35 |
| 19 | CLC | Charcot-Leyden crystal galectin | 105205 | 006659 | 213921 | 7, 15, 36 |
| 19 | CHMP2A | charged multivesicular body protein 2A | 130724 | 130726 | 130725 | 11, 31, 35 |
| 19 | CGB1 | chorionic gonadotropin beta subunit 1 | 267631 | 104818 | 189052 | 32, 15, 12 |
| 19 | CGB2 | chorionic gonadotropin beta subunit 2 | 104818 | 267335 | 267631 | 38, 32, |
| 19 | CGB3 | chorionic gonadotropin beta subunit 3 | 104827 | 268655 | 267335 | 32, 12, 38 |
| 19 | CGB5 | chorionic gonadotropin beta subunit 5 | 189052 | 267631 | 213030 | 32, 12, 38 |
| 19 | CGB7 | chorionic gonadotropin beta subunit 7 | 196337 | 213030 | 283663 | |
| 19 | CGB8 | chorionic gonadotropin beta subunit 8 | 213030 | 189052 | 196337 | 32, 15, 14 |
| 19 | CHAF1A | chromatin assembly factor 1 subunit A | 167670 | 141985 | 167671 | 19, 2, 22 |
| 19 | C19orf12 | chromosome 19 open reading frame 12 | 131943 | 166289 | 105173 | 1, 45, 27 |
| 19 | C19orf18 | chromosome 19 open reading frame 18 | 177025 | 152454 | 166704 | 33, 6, 38 |
| 19 | C19orf24 | chromosome 19 open reading frame 24 | 228300 | 099622 | 099617 | 31, 41, 35 |
| 19 | C19orf25 | chromosome 19 open reading frame 25 | 119559 | 115266 | 115257 | 9, 37, 36 |
| 19 | C19orf33 | chromosome 19 open reading frame 33 | 167644 | 267748 | 167645 | 27, 7, 4 |
| 19 | C19orf35 | chromosome 19 open reading frame 35 | 188305 | 273734 | 220008 | 22, 5, |
| 19 | C19orf38 | chromosome 19 open reading frame 38 | 214212 | 099203 | 142453 | 5, 2, 38 |
| 19 | C19orf43 | chromosome 19 open reading frame 43 | 123144 | 105576 | 198356 | 41, 34, 23 |
| 19 | C19orf44 | chromosome 19 open reading frame 44 | 105072 | 141979 | 085872 | 38, 22, 44 |
| 19 | C19orf45 | chromosome 19 open reading frame 45 | 198723 | 104883 | 267952 | 38, 14, 16 |
| 19 | C19orf47 | chromosome 19 open reading frame 47 | 160392 | 105221 | 105223 | |
| 19 | C19orf48 | chromosome 19 open reading frame 48 | 167747 | 142513 | 167748 | 9, 21, 33 |
| 19 | C19orf53 | chromosome 19 open reading frame 53 | 104979 | 037757 | 280221 | 31, 34, 29 |
| 19 | C19orf54 | chromosome 19 open reading frame 54 | 188493 | 086544 | 077312 | 26, 33, 34 |
| 19 | C19orf57 | chromosome 19 open reading frame 57 | 132016 | 187556 | 132024 | 36, 23, 38 |
| 19 | C19orf60 | chromosome 19 open reading frame 60 | 006015 | 006016 | 105696 | 33, 17, 27 |
| 19 | C19orf66 | chromosome 19 open reading frame 66 | 130813 | 080511 | 130812 | 36, 26, 2 |
| 19 | C19orf67 | chromosome 19 open reading frame 67 | 188032 | 141854 | 141858 | |
| 19 | C19orf68 | chromosome 19 open reading frame 68 | 185453 | 105486 | 178150 | 31, 2, 37 |
| 19 | C19orf70 | chromosome 19 open reading frame 70 | 174917 | 130255 | 167733 | 33, 34, 28 |
| 19 | C19orf71 | chromosome 19 open reading frame 71 | 183397 | 161091 | 064961 | 38, 40, 32 |
| 19 | C19orf73 | chromosome 19 open reading frame 73 | 221916 | 104863 | 177380 | 28, 20, 23 |
| 19 | C19orf81 | chromosome 19 open reading frame 81 | 235034 | 213023 | 161681 | 14, 33, 17 |
| 19 | C19orf84 | chromosome 19 open reading frame 84 | 262874 | 105370 | 142512 | 27, 12, |
| 19 | CLDND2 | claudin domain containing 2 | 160318 | 269403 | 105374 | 40, 44, 38 |
| 19 | CLASRP | CLK4 associating serine/arginine rich protein | 104859 | 104856 | 170684 | 3, 36, 39 |
| 19 | CLPTM1 | CLPTM1, transmembrane protein | 104853 | 234906 | 104856 | 31, 26, 9 |
| 19 | CARM1 | coactivator associated arginine methyltransferase 1 | 142453 | 214212 | 130733 | 3, 34, 18 |
| 19 | COPE | coatomer protein complex subunit epsilon | 105669 | 223802 | 268193 | 34, 21, 41 |
| 19 | COQ8B | coenzyme Q8B | 123815 | 105245 | 086544 | 21, 12, 7 |
| 19 | CC2D1A | coiled-coil and C2 domain containing 1A | 132024 | 132016 | 132000 | 39, 16, 30 |
| 19 | CCDC105 | coiled-coil domain containing 105 | 160994 | 105143 | 105141 | 38, |
| 19 | CCDC106 | coiled-coil domain containing 106 | 173581 | 171425 | 063244 | 34, 1, 9 |
| 19 | CCDC114 | coiled-coil domain containing 114 | 105479 | 105483 | 142227 | 42, 38, 37 |
| 19 | CCDC124 | coiled-coil domain containing 124 | 007080 | 105641 | 105642 | 34, 21, 35 |
| 19 | CCDC130 | coiled-coil domain containing 130 | 104957 | 141837 | 037757 | 28, 21, 37 |
| 19 | CCDC151 | coiled-coil domain containing 151 | 198003 | 205517 | 130175 | 29, 38, 14 |
| 19 | CCDC155 | coiled-coil domain containing 155 | 161609 | 104901 | 142538 | 38, 39, 21 |
| 19 | CCDC159 | coiled-coil domain containing 159 | 183401 | 105518 | 105520 | 29, 28, 36 |
| 19 | CCDC61 | coiled-coil domain containing 61 | 104983 | 104967 | 008438 | 39, 16, 27 |
| 19 | CCDC8 | coiled-coil domain containing 8 | 169515 | 011485 | 277531 | 27, 11, 29 |
| 19 | CCDC9 | coiled-coil domain containing 9 | 105321 | 105327 | 257704 | 18, 40, 3 |
| 19 | CCDC94 | coiled-coil domain containing 94 | 105248 | 105246 | 105251 | 13, 1, 26 |
| 19 | CCDC97 | coiled-coil domain containing 97 | 142039 | 105329 | 255730 | 21, 4, 13 |
| 19 | CCER2 | coiled-coil glutamate rich protein 2 | 262484 | 104825 | 104835 | 14, 37, 25 |
| 19 | CIRBP | cold inducible RNA binding protein | 099622 | 167470 | 228300 | |
| 19 | COLGALT1 | collagen beta(1-O)galactosyltransferase 1 | 130309 | 167483 | 130477 | 6, 26, 3 |
| 19 | COL5A3 | collagen type V alpha 3 chain | 080573 | 105088 | 080511 | 26, 23, 6 |
| 19 | C3 | complement C3 | 125730 | 125735 | 125734 | 1, 27, 19 |
| 19 | C5AR1 | complement C5a receptor 1 | 197405 | 257704 | 134830 | 34, 5, 40 |
| 19 | C5AR2 | complement component 5a receptor 2 | 134830 | 197405 | 134815 | 1, 42, 36 |
| 19 | CFD | complement factor D | 197766 | 197561 | 175221 | 1, 38, 26 |
| 19 | CRX | cone-rod homeobox | 105392 | 178928 | 105398 | |
| 19 | CNFN | cornifelin | 105427 | 105429 | 079435 | 18, 33, 30 |
| 19 | CTXN1 | cortexin 1 | 178531 | 104976 | 104980 | 31, 34, 33 |
| 19 | CKM | creatine kinase, M-type | 104879 | 130201 | 104892 | 25, 15, 18 |
| 19 | CRTC1 | CREB regulated transcription coactivator 1 | 105662 | 167487 | 105664 | 8, 43, 26 |
| 19 | CRB3 | crumbs 3, cell polarity complex component | 130545 | 125648 | 205744 | 28, 42, 37 |
| 19 | CLEC11A | C-type lectin domain family 11 member A | 105472 | 161681 | 142511 | 6, 1, 10 |
| 19 | CLEC17A | C-type lectin domain family 17 member A | 187912 | 099795 | 131355 | 5, 44, |
| 19 | CLEC4G | C-type lectin domain family 4 member G | 182566 | 104921 | 090659 | 28, 32, 19 |
| 19 | CLEC4M | C-type lectin domain family 4 member M | 104938 | 090659 | 142459 | 32, 19, 2 |
| 19 | CELF5 | CUGBP, Elav-like family member 5 | 161082 | 125912 | 141905 | 14, 8, 20 |
| 19 | CXCL17 | C-X-C motif chemokine ligand 17 | 189377 | 079435 | 079385 | 30, 18, 37 |
| 19 | CDKN2D | cyclin dependent kinase inhibitor 2D | 129355 | 129347 | 129354 | 5, 28, 38 |
| 19 | CCNE1 | cyclin E1 | 105173 | 131943 | 105176 | 32, 3, 15 |
| 19 | CNTD2 | cyclin N-terminal domain containing 2 | 105219 | 174521 | 105221 | |
| 19 | COX6B1 | cytochrome c oxidase subunit 6B1 | 126267 | 105672 | 105668 | 29, 25, 15 |
| 19 | COX6B2 | cytochrome c oxidase subunit 6B2 | 160471 | 133247 | 267706 | |
| 19 | COX7A1 | cytochrome c oxidase subunit 7A1 | 161281 | 126247 | 196357 | 25, 15, 29 |
| 19 | CYP2A13 | cytochrome P450 family 2 subfamily A member 13 | 197838 | 197408 | 197446 | 20, |
| 19 | CYP2A6 | cytochrome P450 family 2 subfamily A member 6 | 255974 | 268797 | 198077 | 19, 23, 20 |
| 19 | CYP2A7 | cytochrome P450 family 2 subfamily A member 7 | 198077 | 255974 | 197408 | 19, 23, 40 |
| 19 | CYP2B6 | cytochrome P450 family 2 subfamily B member 6 | 197408 | 198077 | 197838 | 19, 23, 16 |
| 19 | CYP2F1 | cytochrome P450 family 2 subfamily F member 1 | 197446 | 197838 | 167600 | 30, 20, 42 |
| 19 | CYP2S1 | cytochrome P450 family 2 subfamily S member 1 | 167600 | 197446 | 167601 | 3, 24, 13 |
| 19 | CYP4F11 | cytochrome P450 family 4 subfamily F member 11 | 171903 | 186115 | 176231 | 13, 24, 31 |
| 19 | CYP4F12 | cytochrome P450 family 4 subfamily F member 12 | 186204 | 186529 | 171942 | 3, 13, 42 |
| 19 | CYP4F2 | cytochrome P450 family 4 subfamily F member 2 | 186115 | 186723 | 171903 | 17, 19, 16 |
| 19 | CYP4F22 | cytochrome P450 family 4 subfamily F member 22 | 171954 | 161031 | 186526 | 36, 33, 35 |
| 19 | CYP4F3 | cytochrome P450 family 4 subfamily F member 3 | 186529 | 186526 | 186204 | 13, 24, 18 |
| 19 | CYP4F8 | cytochrome P450 family 4 subfamily F member 8 | 186526 | 171954 | 186529 | 33, 35, 15 |
| 19 | CYTH2 | cytohesin 2 | 105443 | 268465 | 142235 | 27, 31, 9 |
| 19 | CRLF1 | cytokine receptor like factor 1 | 006016 | 221983 | 006015 | 19, 24, 10 |
| 19 | CTU1 | cytosolic thiouridylase subunit 1 | 142544 | 129437 | 129450 | 9, 28, 6 |
| 19 | DAND5 | DAN domain BMP antagonist family member 5 | 179284 | 179271 | 008441 | 15, 14, 38 |
| 19 | DAZAP1 | DAZ associated protein 1 | 071626 | 130005 | 115268 | 12, 9, 31 |
| 19 | DBP | D-box binding PAR bZIP transcription factor | 105516 | 063176 | 063180 | 14, 6, 2 |
| 19 | DCAF15 | DDB1 and CUL4 associated factor 15 | 132017 | 132000 | 132005 | 28, 27, 38 |
| 19 | DDX49 | DEAD-box helicase 49 | 105671 | 268193 | 051128 | 13, 43, 34 |
| 19 | DAPK3 | death associated protein kinase 3 | 167657 | 077009 | 167658 | 45, 13, 24 |
| 19 | DEDD2 | death effector domain containing 2 | 160570 | 028277 | 167625 | 41, 29, 12 |
| 19 | DOCK6 | dedicator of cytokinesis 6 | 130158 | 197256 | 130173 | 43, 23, 16 |
| 19 | DLL3 | delta like canonical Notch ligand 3 | 090932 | 105197 | 186838 | 35, 8, 14 |
| 19 | DENND1C | DENN domain containing 1C | 205744 | 130545 | 104833 | 36, 39, 5 |
| 19 | DOHH | deoxyhypusine hydroxylase/monooxygenase | 129932 | 095932 | 105325 | 21, 27, 29 |
| 19 | DHPS | deoxyhypusine synthase | 095059 | 105583 | 132004 | 39, 36, 2 |
| 19 | DNASE2 | deoxyribonuclease 2, lysosomal | 105612 | 105613 | 105610 | 1, 9, 43 |
| 19 | DMKN | dermokine | 161249 | 188508 | 189001 | 35, 15, 29 |
| 19 | DDA1 | DET1 and DDB1 associated 1 | 130311 | 130312 | 074855 | 26, 34, 21 |
| 19 | DDX39A | DExD-box helicase 39A | 123136 | 123146 | 123143 | 21, 38, 41 |
| 19 | DHX34 | DExH-box helicase 34 | 134815 | 134830 | 105419 | 21, 5, 38 |
| 19 | DKKL1 | dickkopf like acrosomal protein 1 | 104901 | 074219 | 161609 | 38, 14, 10 |
| 19 | DHDH | dihydrodiol dehydrogenase | 104808 | 104805 | 087088 | 17, 6, 44 |
| 19 | DUS3L | dihydrouridine synthase 3 like | 141994 | 267157 | 171119 | 22, 21, 23 |
| 19 | DPP9 | dipeptidyl peptidase 9 | 142002 | 074842 | 141965 | 35, 42, 28 |
| 19 | DIRAS1 | DIRAS family GTPase 1 | 176490 | 176533 | 267001 | 29, 14, 15 |
| 19 | DACT3 | dishevelled binding antagonist of beta catenin 3 | 197380 | 167414 | 105287 | 15, 39, 33 |
| 19 | DPRX | divergent-paired related homeobox | 204595 | 130844 | 142405 | |
| 19 | DMRTC2 | DMRT like family C2 | 142025 | 273111 | 105372 | 38, 8, |
| 19 | LIG1 | DNA ligase 1 | 105486 | 105499 | 185453 | 39, 21, 25 |
| 19 | DNMT1 | DNA methyltransferase 1 | 130816 | 130811 | 267534 | 22, 21, 11 |
| 19 | POLD1 | DNA polymerase delta 1, catalytic subunit | 062822 | 131400 | 142539 | 21, 9, 27 |
| 19 | DNAJB1 | DnaJ heat shock protein family (Hsp40) member B1 | 132002 | 123159 | 099797 | 1, 26, 36 |
| 19 | DOTH | DOT1 like histone lysine methyltransferase | 104885 | 065000 | 104886 | 22, 12, 20 |
| 19 | DUXA | double homeobox A | 258873 | 141946 | 083844 | 6, |
| 19 | DPF1 | double PHD fingers 1 | 011332 | 105738 | 167642 | 26, 34, 8 |
| 19 | DPY19L3 | dpy-19 like 3 (C. elegans) | 178904 | 168813 | 105185 | 29, 12, 22 |
| 19 | DYRK1B | dual specificity tyrosine phosphorylation regulated kinase 1B | 105204 | 213921 | 105202 | 38, 26, 9 |
| 19 | DNM2 | dynamin 2 | 079805 | 213339 | 099203 | |
| 19 | DNAAF3 | dynein axonemal assembly factor 3 | 167646 | 267110 | 129990 | 38, 10, 42 |
| 19 | DMPK | dystrophia myotonica protein kinase | 104936 | 177045 | 268434 | 34, 15, 26 |
| 19 | DMWD | dystrophia myotonica, WD repeat containing | 185800 | 268434 | 104941 | 26, 14, 41 |
| 19 | EML2 | echinoderm microtubule associated protein like 2 | 125746 | 177464 | 010310 | 21, 1, 36 |
| 19 | EVI5L | ecotropic viral integration site 5 like | 142459 | 104938 | 183248 | 26, 31, 14 |
| 19 | ECSIT | ECSIT signalling integrator | 130159 | 161914 | 130176 | 21, 1, 9 |
| 19 | EGLN2 | egl-9 family hypoxia inducible factor 2 | 269858 | 171570 | 268797 | 22, 36, 38 |
| 19 | EHD2 | EH domain containing 2 | 024422 | 063169 | 105373 | 42, 44, 24 |
| 19 | ELANE | elastase, neutrophil expressed | 197561 | 196415 | 197766 | 7, 5, 9 |
| 19 | ELAVL1 | ELAV like RNA binding protein 1 | 066044 | 104980 | 131142 | 29, 26, 4 |
| 19 | ELAVL3 | ELAV like RNA binding protein 3 | 196361 | 130175 | 267477 | 14, 45, 8 |
| 19 | ETFB | electron transfer flavoprotein beta subunit | 105379 | 186806 | 269403 | 25, 14, 35 |
| 19 | ELOF1 | elongation factor 1 homolog | 130165 | 130176 | 198551 | 1, 26, 27 |
| 19 | ELL | elongation factor for RNA polymerase II | 105656 | 105655 | 105701 | 21, 5, 16 |
| 19 | ERVK3-1 | Endogenous retrovirus group K3 member 1 | 142396 | 267216 | 182318 | |
| 19 | ERVV-1 | Endogenous retrovirus group V member 1 | 269526 | 180257 | 268964 | 32, |
| 19 | ERVV-2 | Endogenous retrovirus group V member 2 | 268964 | 269526 | 170949 | 32, 6, 22 |
| 19 | ECH1 | enoyl-CoA hydratase 1 | 104823 | 171747 | 268083 | 30, 1, 25 |
| 19 | EID2 | EP300 interacting inhibitor of differentiation 2 | 176396 | 176401 | 105198 | 6, 4, 34 |
| 19 | EID2B | EP300 interacting inhibitor of differentiation 2B | 176401 | 186838 | 176396 | 11, 34, 13 |
| 19 | EFNA2 | ephrin A2 | 099617 | 228300 | 160953 | 6, 16, 14 |
| 19 | EPS15L1 | epidermal growth factor receptor pathway substrate 15 like 1 | 127527 | 127528 | 269058 | 43, 2, 36 |
| 19 | ELSPBP1 | epididymal sperm binding protein 1 | 169393 | 188334 | 105507 | 14, 38, 10 |
| 19 | EMP3 | epithelial membrane protein 3 | 142227 | 105479 | 161558 | 6, 43, 1 |
| 19 | EPHX3 | epoxide hydrolase 3 | 105131 | 074181 | 141867 | 13, 22, 32 |
| 19 | EPS8L1 | EPS8 like 1 | 131037 | 160439 | 125503 | 29, 32, 27 |
| 19 | EPN1 | epsin 1 | 063245 | 063244 | 185792 | 17, |
| 19 | EBI3 | Epstein-Barr virus induced 3 | 105246 | 089847 | 105248 | 32, 41, 9 |
| 19 | EMC10 | ER membrane protein complex subunit 10 | 161671 | 142530 | 161677 | 31, 11, 12 |
| 19 | ERCC1 | ERCC excision repair 1, endonuclease non-catalytic subunit | 012061 | 117877 | 125740 | 5, 33, 6 |
| 19 | ERCC2 | ERCC excision repair 2, TFIIH core complex helicase subunit | 104884 | 104892 | 104881 | 21, 3, 31 |
| 19 | EPOR | erythropoietin receptor | 187266 | 173928 | 205517 | 2, 1, 32 |
| 19 | ETHE1 | ETHE1, persulfide dioxygenase | 105755 | 176531 | 176472 | 43, 16, 4 |
| 19 | ETV2 | ETS variant 2 | 105672 | 126254 | 126267 | 38, 32, 9 |
| 19 | ERF | ETS2 repressor factor | 105722 | 268643 | 079432 | 26, 40, 43 |
| 19 | EEF2 | eukaryotic translation elongation factor 2 | 167658 | 167657 | 105229 | 33, 40, 24 |
| 19 | EIF3G | eukaryotic translation initiation factor 3 subunit G | 130811 | 244165 | 130816 | 34, 1, 35 |
| 19 | EIF3K | eukaryotic translation initiation factor 3 subunit K | 178982 | 104814 | 130402 | 27, 7, 29 |
| 19 | EXOC3L2 | exocyst complex component 3 like 2 | 130201 | 283632 | 104879 | 40, 28, 14 |
| 19 | EXOC3L2 | exocyst complex component 3 like 2 | 283632 | 267545 | 130201 | 40, 28, 14 |
| 19 | EXOSC5 | exosome component 5 | 077348 | 123810 | 248098 | 21, 41, 9 |
| 19 | F2RL3 | F2R like thrombin/trypsin receptor 3 | 127533 | 127511 | 160111 | 7, 28, 16 |
| 19 | FAM129C | family with sequence similarity 129 member C | 167483 | 130313 | 130309 | 36, 21, 22 |
| 19 | FAM187B | family with sequence similarity 187 member B | 177558 | 089327 | 105699 | 38, 44, 8 |
| 19 | FAM32A | family with sequence similarity 32 member A | 105058 | 141977 | 072958 | 12, 6, 21 |
| 19 | FAM71E1 | family with sequence similarity 71 member E1 | 142530 | 086967 | 161671 | 38, 14, 21 |
| 19 | FAM71E2 | family with sequence similarity 71 member E2 | 180043 | 267706 | 095752 | 38, 39, 23 |
| 19 | FAM83E | family with sequence similarity 83 member E | 105523 | 088002 | 177202 | 37, 16, 24 |
| 19 | FAM98C | family with sequence similarity 98 member C | 130244 | 188766 | 171777 | 28, 41, 20 |
| 19 | FAAP24 | Fanconi anemia core complex associated protein 24 | 131944 | 121289 | 131941 | 4, 2, 30 |
| 19 | FBXL12 | F-box and leucine rich repeat protein 12 | 127452 | 196605 | 198258 | 39, 37, 9 |
| 19 | FBXW9 | F-box and WD repeat domain containing 9 | 132004 | 095059 | 105576 | 34, 3, 21 |
| 19 | FBXO17 | F-box protein 17 | 269190 | 128626 | 161243 | 6, 28, 12 |
| 19 | FBXO27 | F-box protein 27 | 161243 | 269190 | 183760 | 28, 17, 7 |
| 19 | FBXO46 | F-box protein 46 | 177051 | 011478 | 237452 | 26, 22, 44 |
| 19 | FCAR | Fc fragment of IgA receptor | 186431 | 240403 | 189430 | 5, 7, 10 |
| 19 | FCER2 | Fc fragment of IgE receptor II | 104921 | 181029 | 182566 | 41, 36, 22 |
| 19 | FCGBP | Fc fragment of IgG binding protein | 275395 | 105202 | 013275 | 3, 16, 21 |
| 19 | FCGRT | Fc fragment of IgG receptor and transporter | 104870 | 142534 | 142552 | 27, 7, 23 |
| 19 | FCHO1 | FCH domain only 1 | 130475 | 130479 | 179913 | 21, 22, 7 |
| 19 | FEM1A | fem-1 homolog A | 141965 | 142002 | 127666 | 34, 35, 29 |
| 19 | FDX2 | ferredoxin 2 | 267673 | 167807 | 267303 | |
| 19 | FTL | ferritin light chain | 087086 | 087088 | 104812 | |
| 19 | FBL | fibrillarin | 105202 | 105204 | 275395 | 28, 27, 21 |
| 19 | FBN3 | fibrillin 3 | 142449 | 131142 | 090661 | 12, 2, 37 |
| 19 | FGF21 | fibroblast growth factor 21 | 105550 | 174951 | 105552 | 20, |
| 19 | FGF22 | fibroblast growth factor 22 | 070388 | 099821 | 070423 | 27, 8, |
| 19 | FSD1 | fibronectin type III and SPRY domain containing 1 | 105255 | 167664 | 178078 | 8, 14, 26 |
| 19 | FZR1 | fizzy/cell division cycle 20 related 1 | 105325 | 129932 | 161091 | 21, 34, 29 |
| 19 | FKBP8 | FK506 binding protein 8 | 105701 | 105656 | 105700 | 32, |
| 19 | FIZ1 | FLT3 interacting zinc finger 1 | 179943 | 218891 | 171443 | 34, 29, 25 |
| 19 | FLT3LG | fms related tyrosine kinase 3 ligand | 090554 | 269469 | 142541 | 14, 36, 39 |
| 19 | FSTL3 | follistatin like 3 | 070404 | 070423 | 185198 | 32, 26, 38 |
| 19 | FOXA3 | forkhead box A3 | 170608 | 125755 | 170604 | 3, 37, 16 |
| 19 | FPR1 | formyl peptide receptor 1 | 171051 | 105509 | 171049 | 5, 36, 14 |
| 19 | FPR2 | formyl peptide receptor 2 | 171049 | 171051 | 187474 | 5, 36, 15 |
| 19 | FPR3 | formyl peptide receptor 3 | 187474 | 171049 | 161551 | 29, 10, 5 |
| 19 | FOSB | FosB proto-oncogene, AP-1 transcription factor subunit | 125740 | 012061 | 125744 | 42, 1, 44 |
| 19 | FFAR1 | free fatty acid receptor 1 | 126266 | 012124 | 185897 | 28, 44, 8 |
| 19 | FFAR2 | free fatty acid receptor 2 | 126262 | 126251 | 188508 | 3, 28, 20 |
| 19 | FFAR3 | free fatty acid receptor 3 | 185897 | 126266 | 126251 | 27, 5, 16 |
| 19 | FUT1 | fucosyltransferase 1 (H blood group) | 174951 | 182264 | 105550 | 29, 37, 9 |
| 19 | FUT2 | fucosyltransferase 2 | 176920 | 142233 | 176909 | |
| 19 | FUT3 | fucosyltransferase 3 (Lewis blood group) | 171124 | 156413 | 130383 | 3, 42, 16 |
| 19 | FUT5 | fucosyltransferase 5 | 130383 | 171124 | 267740 | 38, |
| 19 | FUT6 | fucosyltransferase 6 | 156413 | 171119 | 171124 | |
| 19 | FKRP | fukutin related protein | 181027 | 090372 | 105281 | 18, 44, |
| 19 | FUZ | fuzzy planar cell polarity protein | 010361 | 196961 | 104973 | 21, 38, 26 |
| 19 | FXYD1 | FXYD domain containing ion transport regulator 1 | 266964 | 153902 | 221946 | 25, 15, 14 |
| 19 | FXYD3 | FXYD domain containing ion transport regulator 3 | 089356 | 105707 | 153902 | 4, 16, 33 |
| 19 | FXYD5 | FXYD domain containing ion transport regulator 5 | 089327 | 221946 | 177558 | 34, 41, 37 |
| 19 | FXYD7 | FXYD domain containing ion transport regulator 7 | 221946 | 266964 | 089327 | 8, 44, 10 |
| 19 | GNA11 | G protein subunit alpha 11 | 088256 | 104964 | 060558 | |
| 19 | GNA15 | G protein subunit alpha 15 | 060558 | 088256 | 125910 | 39, 13, 24 |
| 19 | GNG7 | G protein subunit gamma 7 | 176533 | 099860 | 176490 | |
| 19 | GNG8 | G protein subunit gamma 8 | 167414 | 160013 | 197380 | |
| 19 | GPR108 | G protein-coupled receptor 108 | 125734 | 125730 | 125733 | 9, 24, 4 |
| 19 | GPR32 | G protein-coupled receptor 32 | 142511 | 105472 | 261341 | 32, |
| 19 | GPR4 | G protein-coupled receptor 4 | 177464 | 125741 | 125746 | 43, 45, 4 |
| 19 | GPR42 | G protein-coupled receptor 42 (gene/pseudogene) | 126251 | 185897 | 126262 | |
| 19 | GADD45GIP1 | GADD45G interacting protein 1 | 179271 | 179262 | 179284 | 20, 23, 16 |
| 19 | GALP | galanin like peptide | 197487 | 167685 | 197213 | 12, |
| 19 | LGALS13 | galectin 13 | 105198 | 176396 | 249861 | 32, |
| 19 | LGALS14 | galectin 14 | 006659 | 249861 | 105205 | 32, 45, 27 |
| 19 | LGALS16 | galectin 16 | 249861 | 105198 | 006659 | 32, 12, |
| 19 | LGALS4 | galectin 4 | 171747 | 178934 | 104823 | 16, 3, 37 |
| 19 | LGALS7 | galectin 7 | 205076 | 182472 | 178934 | 15, 26, 18 |
| 19 | LGALS7B | galectin 7B | 178934 | 205076 | 171747 | 15, 33, 10 |
| 19 | GGN | gametogenetin | 179168 | 099341 | 188766 | 38, 21, 19 |
| 19 | GIPR | gastric inhibitory polypeptide receptor | 010310 | 125746 | 125743 | 28, 16, 12 |
| 19 | GATAD2A | GATA zinc finger domain containing 2A | 167491 | 129933 | 178093 | 22, 16, 21 |
| 19 | GMIP | GEM interacting protein | 089639 | 064547 | 105726 | 29, 22, 9 |
| 19 | GEMIN7 | gem nuclear organelle associated protein 7 | 142252 | 170684 | 007047 | 41, 34, 25 |
| 19 | GTF2F1 | general transcription factor IIF subunit 1 | 125651 | 125650 | 088247 | 2, 14, 40 |
| 19 | GIPC1 | GIPC PDZ domain containing family member 1 | 123159 | 160951 | 132002 | 35, 28, 34 |
| 19 | GIPC3 | GIPC PDZ domain containing family member 3 | 179855 | 064961 | 006638 | 43, 40, 14 |
| 19 | GMFG | glia maturation factor gamma | 130755 | 128011 | 179134 | 7, 29, 30 |
| 19 | GLTSCR1 | glioma tumor suppressor candidate region gene 1 | 063169 | 118156 | 024422 | 21, 12, 9 |
| 19 | GLTSCR2 | glioma tumor suppressor candidate region gene 2 | 105373 | 024422 | 178980 | 21, 41, 22 |
| 19 | GPI | glucose-6-phosphate isomerase | 105220 | 166398 | 266953 | 13, 7, 2 |
| 19 | GRIK5 | glutamate ionotropic receptor kainate type subunit 5 | 105737 | 105409 | 105732 | 26, 22, 12 |
| 19 | GRIN2D | glutamate ionotropic receptor NMDA type subunit 2D | 105464 | 105438 | 105447 | 6, 23, 16 |
| 19 | GRIN3B | glutamate ionotropic receptor NMDA type subunit 3B | 116032 | 065268 | 182087 | 20, 12, |
| 19 | ERICH4 | glutamate rich 4 | 204978 | 105341 | 007129 | 17, 20, |
| 19 | GRWD1 | glutamate rich WD repeat containing 1 | 105447 | 105464 | 182324 | 36, 21, 45 |
| 19 | QPCTL | glutaminyl-peptide cyclotransferase like | 011478 | 125743 | 177051 | 18, 9, 37 |
| 19 | GCDH | glutaryl-CoA dehydrogenase | 105607 | 105610 | 161860 | |
| 19 | GPX4 | glutathione peroxidase 4 | 167468 | 099817 | 064932 | 29, 1, 15 |
| 19 | GAPDHS | glyceraldehyde-3-phosphate dehydrogenase, spermatogenic | 105679 | 189001 | 105677 | 38, 35, 12 |
| 19 | GYS1 | glycogen synthase 1 | 104812 | 087086 | 183207 | 24, 43, 42 |
| 19 | GSK3A | glycogen synthase kinase 3 alpha | 105723 | 167625 | 268643 | 34, 27, 29 |
| 19 | GP6 | glycoprotein VI platelet | 088053 | 022556 | 160439 | 30, 25, 24 |
| 19 | GFY | golgi associated olfactory signaling regulator | 261949 | 142538 | 104888 | |
| 19 | GPATCH1 | G-patch domain containing 1 | 076650 | 131941 | 166359 | 45, 1, 31 |
| 19 | GRAMD1A | GRAM domain containing 1A | 089351 | 180884 | 105711 | 36, 34, 33 |
| 19 | GZMM | granzyme M | 197540 | 099804 | 172270 | 45, 39, 16 |
| 19 | GADD45B | growth arrest and DNA damage inducible beta | 099860 | 176619 | 176533 | 3, 1, 20 |
| 19 | GDF1 | growth differentiation factor 1 | 130283 | 005007 | 105669 | |
| 19 | GDF15 | growth differentiation factor 15 | 130513 | 130517 | 175489 | 20, 19, 14 |
| 19 | GTPBP3 | GTP binding protein 3 (mitochondrial) | 130299 | 074855 | 130300 | |
| 19 | GAMT | guanidinoacetate N-methyltransferase | 130005 | 115286 | 071626 | 25, 19, 21 |
| 19 | HAUS5 | HAUS augmin like complex subunit 5 | 249115 | 105675 | 126254 | 26, 9, 5 |
| 19 | HAUS8 | HAUS augmin like complex subunit 8 | 131351 | 160111 | 099331 | 9, 35, 6 |
| 19 | HSPB6 | heat shock protein family B (small) member 6 | 004776 | 267796 | 167595 | 25, 1, 15 |
| 19 | HCST | hematopoietic cell signal transducer | 126264 | 167604 | 011600 | 7, 43, 37 |
| 19 | HSH2D | hematopoietic SH2 domain containing | 196684 | 167461 | 141977 | 5, 7, 4 |
| 19 | HAMP | hepcidin antimicrobial peptide | 105697 | 105698 | 105695 | 36, 19, 14 |
| 19 | HPN | hepsin | 105707 | 105711 | 089356 | 19, 41, 40 |
| 19 | HNRNPL | heterogeneous nuclear ribonucleoprotein L | 104824 | 268083 | 187994 | 30, 33, 39 |
| 19 | HNRNPM | heterogeneous nuclear ribonucleoprotein M | 099783 | 099785 | 133246 | 12, 31, 3 |
| 19 | HNRNPUL1 | heterogeneous nuclear ribonucleoprotein U like 1 | 105323 | 167601 | 105329 | 41, 21, 39 |
| 19 | HMG20B | high mobility group 20B | 064961 | 183397 | 179855 | 35, 3, 34 |
| 19 | HRC | histidine rich calcium binding protein | 130528 | 177380 | 130529 | 15, 25, 11 |
| 19 | HKR1 | HKR1, GLI-Kruppel zinc finger family member | 181666 | 188283 | 189164 | |
| 19 | HIPK4 | homeodomain interacting protein kinase 4 | 160396 | 105223 | 105227 | 38, 2, 33 |
| 19 | HOMER3 | homer scaffolding protein 3 | 051128 | 105671 | 064607 | 13, 19, 40 |
| 19 | HOOK2 | hook microtubule tethering protein 2 | 095066 | 039987 | 171223 | 1, 40, 13 |
| 19 | HSPBP1 | HSPA (Hsp70) binding protein 1 | 133265 | 105063 | 160469 | 9, 21, 3 |
| 19 | HAPLN4 | hyaluronan and proteoglycan link protein 4 | 187664 | 130287 | 213996 | 14, 15, 24 |
| 19 | HAS1 | hyaluronan synthase 1 | 105509 | 182310 | 171051 | 1, 32, 6 |
| 19 | HSD11B1L | hydroxysteroid 11-beta dehydrogenase 1 like | 167733 | 174917 | 196365 | |
| 19 | HSD17B14 | hydroxysteroid 17-beta dehydrogenase 14 | 087076 | 105552 | 105559 | 11, 17, 20 |
| 19 | HCN2 | hyperpolarization activated cyclic nucleotide gated | 099822 | 172270 | 099821 | 29, 28, 9 |
| | | potassium channel 2 | | | | |
| 19 | HIF3A | hypoxia inducible factor 3 alpha subunit | 124440 | 188293 | 011485 | 36, 26, 15 |
| 19 | IFI30 | IFI30, lysosomal thiol reductase | 216490 | 268173 | 254858 | 41, 21, 30 |
| 19 | IGFL1 | IGF like family member 1 | 188293 | 204866 | 124440 | 24, 44, 33 |
| 19 | IGFL2 | IGF like family member 2 | 204866 | 188624 | 188293 | 24, 38, 16 |
| 19 | IGFL3 | IGF like family member 3 | 188624 | 204869 | 204866 | 6, 15, 35 |
| 19 | IGFL4 | IGF like family member 4 | 204869 | 008438 | 188624 | |
| 19 | IGFLR1 | IGF like family receptor 1 | 126246 | 272333 | 267120 | 43, 6, 14 |
| 19 | IGLON5 | IgLON family member 5 | 142549 | 179213 | 186806 | 2, 14, 12 |
| 19 | ILVBL | ilvB acetolactate synthase like | 105135 | 105137 | 074181 | 21, 29, 3 |
| 19 | IER2 | immediate early response 2 | 160888 | 104915 | 141837 | 32, 29, 27 |
| 19 | IRGC | immunity related GTPase cinema | 124449 | 011422 | 105771 | 38, 32, 8 |
| 19 | IRGQ | immunity related GTPase Q | 167378 | 234465 | 124444 | 18, 32, 26 |
| 19 | IGSF23 | immunoglobulin superfamily member 23 | 216588 | 273777 | 073008 | 10, 38, 16 |
| 19 | INAFM1 | InaF motif containing 1 | 257704 | 105321 | 197405 | |
| 19 | ISYNA1 | inositol-3-phosphate synthase 1 | 105655 | 130511 | 105656 | |
| 19 | ITPKC | inositol-trisphosphate 3-kinase C | 086544 | 123815 | 188493 | 18, 43, 30 |
| 19 | INSL3 | insulin like 3 | 248099 | 179913 | 105639 | 38, 8, 5 |
| 19 | INSR | insulin receptor | 171105 | 130544 | 263264 | 1, 32, 40 |
| 19 | ICAM1 | intercellular adhesion molecule 1 | 090339 | 105364 | 105371 | 36, 20, 1 |
| 19 | ICAM3 | intercellular adhesion molecule 3 | 076662 | 161847 | 105397 | 36, 5, 7 |
| 19 | ICAM4 | intercellular adhesion molecule 4 (Landsteiner-Wiener | 105371 | 090339 | 105376 | 5, 20, 12 |
| | | blood group) | | | | |
| 19 | ICAM5 | intercellular adhesion molecule 5 | 105376 | 105371 | 220201 | 40, 7, 8 |
| 19 | IFNL1 | interferon lambda 1 | 182393 | 183709 | 128011 | |
| 19 | IFNL2 | interferon lambda 2 | 183709 | 272395 | 182393 | 14, |
| 19 | IFNL3 | interferon lambda 3 | 197110 | 179751 | 272395 | |
| 19 | IFNL4 | interferon lambda 4 (gene/pseudogene) | 272395 | 197110 | 183709 | |
| 19 | IRF2BP1 | interferon regulatory factor 2 binding protein 1 | 170604 | 170608 | 176182 | 36, 28, 34 |
| 19 | IRF3 | interferon regulatory factor 3 | 126456 | 126461 | 126453 | 21, 41, 28 |
| 19 | IL11 | interleukin 11 | 095752 | 180043 | 160472 | 45, 35, 28 |
| 19 | IL12RB1 | interleukin 12 receptor subunit beta 1 | 096996 | 105643 | 099308 | |
| 19 | IL27RA | interleukin 27 receptor subunit alpha | 104998 | 171136 | 187867 | 40, 41, 22 |
| 19 | IL4I1 | interleukin 4 induced 1 | 104951 | 104946 | 213024 | 26, 38, 32 |
| 19 | ILF3 | interleukin enhancer binding factor 3 | 129351 | 129353 | 213339 | 12, 21, 22 |
| 19 | ISOC2 | isochorismatase domain containing 2 | 063241 | 187902 | 283567 | 41, 21, 34 |
| 19 | IZUMO2 | IZUMO family member 2 | 161652 | 142528 | 105357 | 38, |
| 19 | IZUMO4 | IZUMO family member 4 | 099840 | 172081 | 065000 | 38, 13, 8 |
| 19 | IZUMO1 | izumo sperm-egg fusion 1 | 182264 | 105538 | 174951 | 26, 38, 7 |
| 19 | JAK3 | Janus kinase 3 | 105639 | 248099 | 105640 | 22, 41, 1 |
| 19 | JOSD2 | Josephin domain containing 2 | 161677 | 161671 | 204653 | 29, 6, 3 |
| 19 | JUNB | JunB proto-oncogene, AP-1 transcription factor subunit | 171223 | 095066 | 167815 | 1, 26, 31 |
| 19 | JSRP1 | junctional sarcoplasmic reticulum protein 1 | 167476 | 104899 | 104904 | 20, |
| 19 | JUND | JunD proto-oncogene, AP-1 transcription factor subunit | 130522 | 130518 | 130520 | 41, 27, 17 |
| 19 | KLK1 | kallikrein 1 | 167748 | 167747 | 174562 | 34, 28, 41 |
| 19 | KLK10 | kallikrein related peptidase 10 | 129451 | 213022 | 167757 | 3, 18, 24 |
| 19 | KLK11 | kallikrein related peptidase 11 | 167757 | 129451 | 186474 | 27, 1, 31 |
| 19 | KLK12 | kallikrein related peptidase 12 | 186474 | 167757 | 167759 | 24, 38, |
| 19 | KLK13 | kallikrein related peptidase 13 | 167759 | 186474 | 129437 | 30, 24, 38 |
| 19 | KLK14 | kallikrein related peptidase 14 | 129437 | 167759 | 142544 | 15, 20, |
| 19 | KLK15 | kallikrein related peptidase 15 | 174562 | 167748 | 142515 | 13, 6, 33 |
| 19 | KLK2 | kallikrein related peptidase 2 | 167751 | 142515 | 167749 | 33, 26, 6 |
| 19 | KLK3 | kallikrein related peptidase 3 | 142515 | 174562 | 167751 | 33, 26, 23 |
| 19 | KLK4 | kallikrein related peptidase 4 | 167749 | 167751 | 167754 | 33, 44, 5 |
| 19 | KLK5 | kallikrein related peptidase 5 | 167754 | 167749 | 167755 | 22, 27, 15 |
| 19 | KLK6 | kallikrein related peptidase 6 | 167755 | 167754 | 169035 | 22, 30, 27 |
| 19 | KLK7 | kallikrein related peptidase 7 | 169035 | 167755 | 129455 | 3, 13, 27 |
| 19 | KLK8 | kallikrein related peptidase 8 | 129455 | 169035 | 269741 | 1, 27, 13 |
| 19 | KLK9 | kallikrein related peptidase 9 | 213022 | 269741 | 129451 | 18, 32, |
| 19 | KPTN | kaptin, actin binding protein | 118162 | 118160 | 105402 | 40, 22, 26 |
| 19 | KDELR1 | KDEL endoplasmic reticulum protein retention receptor 1 | 105438 | 105467 | 105464 | 32, 1, 31 |
| 19 | KEAP1 | kelch like ECH associated protein 1 | 079999 | 065989 | 180739 | 21, 24, 3 |
| 19 | KLHL26 | kelch like family member 26 | 167487 | 105696 | 105662 | 27, 37, 39 |
| 19 | KRTDAP | keratinocyte differentiation associated protein | 188508 | 126262 | 161249 | 18, 15, 30 |
| 19 | KHSRP | KH-type splicing regulatory protein | 088247 | 125651 | 181240 | 21, 22, 14 |
| 19 | KIAA0355 | KIAA0355 | 166398 | 257103 | 105220 | 1, 22, 25 |
| 19 | KIAA1683 | KIAA1683 | 130518 | 105650 | 130522 | 38, 36, 29 |
| 19 | KIR3DL1 | killer cell immunoglobulin like receptor, three Ig domains | 167633 | 189013 | 221957 | 25, 19, |
| | | and long cytoplasmic tail 1 | | | | |
| 19 | KIR3DL2 | killer cell immunoglobulin like receptor, three Ig domains | 240403 | 221957 | 186431 | 5, 23, 28 |
| | | and long cytoplasmic tail 2 | | | | |
| 19 | KIR3DL3 | killer cell immunoglobulin like receptor, three Ig domains | 242019 | 186818 | 243772 | |
| | | and long cytoplasmic tail 3 | | | | |
| 19 | KIR3DX1 | Killer cell immunoglobulin like receptor, three Ig domains X1 | 104970 | 167618 | 239998 | 7, 5, |
| 19 | KIR2DL1 | killer cell immunoglobulin like receptor, two Ig domains | 125498 | 243772 | 189013 | |
| | | and long cytoplasmic tail 1 | | | | |
| 19 | KIR2DL3 | killer cell immunoglobulin like receptor, two Ig domains | 243772 | 242019 | 125498 | 5, 23, 32 |
| | | and long cytoplasmic tail 3 | | | | |
| 19 | KIR2DL4 | killer cell immunoglobulin like receptor, two Ig domains | 189013 | 125498 | 167633 | |
| | | and long cytoplasmic tail 4 | | | | |
| 19 | KIR2DS4 | killer cell immunoglobulin like receptor, two Ig domains | 221957 | 167633 | 240403 | 5, |
| | | and short cytoplasmic tail 4 | | | | |
| 19 | KIRREL2 | kin of IRRE like 2 (Drosophila) | 126259 | 161270 | 105290 | 31, 14, 20 |
| 19 | KLC3 | kinesin light chain 3 | 104892 | 104879 | 104884 | 2, 32, 27 |
| 19 | KISS1R | KISS1 receptor | 116014 | 198858 | 116017 | 17, 32, 16 |
| 19 | KANK2 | KN motif and ankyrin repeat domains 2 | 197256 | 161888 | 130158 | |
| 19 | KANK3 | KN motif and ankyrin repeat domains 3 | 186994 | 233927 | 167772 | 26, 32, 3 |
| 19 | KRI1 | KRI1 homolog | 129347 | 130734 | 129355 | 36, 39, 1 |
| 19 | KLF1 | Kruppel like factor 1 | 105610 | 105612 | 105607 | 5, 16, |
| 19 | KLF16 | Kruppel like factor 16 | 129911 | 079313 | 129968 | 31, 28, 16 |
| 19 | KLF2 | Kruppel like factor 2 | 127528 | 072958 | 127527 | 20, 28, 17 |
| 19 | KXD1 | KxDL motif containing 1 | 105700 | 105701 | 221983 | 29, 27, 3 |
| 19 | LMNB2 | lamin B2 | 176619 | 099800 | 099860 | 34, 12, 22 |
| 19 | LTBP4 | latent transforming growth factor beta binding protein 4 | 090006 | 160410 | 279183 | 41, 37, 27 |
| 19 | LRP3 | LDL receptor related protein 3 | 130881 | 166359 | 130876 | 34, 26, 14 |
| 19 | LMTK3 | lemur tyrosine kinase 3 | 142235 | 105443 | 088002 | 26, 16, 8 |
| 19 | LIM2 | lens intrinsic membrane protein 2 | 105370 | 105374 | 262874 | 14, |
| 19 | LRG1 | leucine rich alpha-2-glycoprotein 1 | 171236 | 267385 | 167680 | 19, 5, 37 |
| 19 | LRFN1 | leucine rich repeat and fibronectin type III domain | 128011 | 182393 | 130755 | 27, 33, 6 |
| | | containing 1 | | | | |
| 19 | LRFN3 | leucine rich repeat and fibronectin type III domain | 126243 | 011600 | 205138 | 35, 22, 3 |
| | | containing 3 | | | | |
| 19 | LINGO3 | leucine rich repeat and Ig domain containing 3 | 220008 | 188305 | 130332 | 36, 5, 20 |
| 19 | LRRC25 | leucine rich repeat containing 25 | 175489 | 130513 | 130511 | 5, 36, 32 |
| 19 | LRRC4B | leucine rich repeat containing 4B | 131409 | 204653 | 213023 | 14, 15, 8 |
| 19 | LRRC8E | leucine rich repeat containing 8 family member E | 171017 | 183248 | 076984 | 18, 24, 4 |
| 19 | LGI4 | leucine rich repeat LGI family member 4 | 153902 | 089356 | 266964 | 26, 1, 14 |
| 19 | LEUTX | leucine twenty homeobox | 213921 | 105205 | 105204 | 32, |
| 19 | LAIR1 | leukocyte associated immunoglobulin like receptor 1 | 167613 | 239961 | 167614 | 39, 36, 5 |
| 19 | LAIR2 | leukocyte associated immunoglobulin like receptor 2 | 167618 | 167617 | 104970 | 32, 44, 10 |
| 19 | LILRA1 | leukocyte immunoglobulin like receptor A1 | 104974 | 239998 | 104972 | 7, 5, 36 |
| 19 | LILRA2 | leukocyte immunoglobulin like receptor A2 | 239998 | 104970 | 104974 | 5, 36, 7 |
| 19 | LILRA4 | leukocyte immunoglobulin like receptor A4 | 239961 | 187116 | 167613 | 36, 22, 7 |
| 19 | LILRA5 | leukocyte immunoglobulin like receptor A5 | 187116 | 131042 | 239961 | 5, 23, |
| 19 | LILRA6 | leukocyte immunoglobulin like receptor A6 | 244482 | 204577 | 105609 | 5, 36, 26 |
| 19 | LILRB1 | leukocyte immunoglobulin like receptor B1 | 104972 | 104974 | 186818 | 36, 7, 41 |
| 19 | LILRB2 | leukocyte immunoglobulin like receptor B2 | 131042 | 105609 | 187116 | 36, 5, 7 |
| 19 | LILRB3 | leukocyte immunoglobulin like receptor B3 | 204577 | 170889 | 244482 | |
| 19 | LILRB4 | leukocyte immunoglobulin like receptor B4 | 186818 | 104972 | 242019 | 10, 5, 13 |
| 19 | LILRB5 | leukocyte immunoglobulin like receptor B5 | 105609 | 244482 | 131042 | 36, 10, 39 |
| 19 | LENG1 | leukocyte receptor cluster member 1 | 105617 | 088038 | 167608 | 6, 14, 44 |
| 19 | LENG8 | leukocyte receptor cluster member 8 | 167615 | 167614 | 275183 | 40, 26, 7 |
| 19 | LENG9 | leukocyte receptor cluster member 9 | 275183 | 167615 | 167617 | 9, 32, 20 |
| 19 | LIN37 | lin-37 DREAM MuvB core complex component | 267796 | 188223 | 004776 | 15, 26, 41 |
| 19 | LIN7B | lin-7 homolog B, crumbs cell polarity complex component | 104863 | 104852 | 221916 | 31, 15, 13 |
| 19 | LIPE | lipase E, hormone sensitive type | 079435 | 105427 | 189377 | 1, 23, 22 |
| 19 | LSR | lipolysis stimulated lipoprotein receptor | 105699 | 177558 | 105698 | 3, 37, 16 |
| 19 | LONP1 | Ion peptidase 1, mitochondrial | 196365 | 167733 | 174898 | 21, 34, 35 |
| 19 | LDLR | low density lipoprotein receptor | 130164 | 127616 | 161888 | 43, 42, 24 |
| 19 | LSM14A | LSM14A, mRNA processing body assembly factor | 257103 | 153885 | 166398 | 11, 1, 12 |
| 19 | LSM4 | LSM4 homolog, U6 small nuclear RNA and mRNA degradation | 130520 | 130522 | 130517 | 29, 27, 41 |
| | | associated | | | | |
| 19 | LSM7 | LSM7 homolog, U6 small nuclear RNA and mRNA degradation | 130332 | 220008 | 005206 | 41, 29, 22 |
| | | associated | | | | |
| 19 | LHB | luteinizing hormone beta polypeptide | 104826 | 183207 | 268655 | 31, 12, 32 |
| 19 | LYPD3 | LY6/PLAUR domain containing 3 | 124466 | 131126 | 176531 | 13, 30, 32 |
| 19 | LYPD4 | LY6/PLAUR domain containing 4 | 273111 | 170956 | 142025 | 38, 10, |
| 19 | LYPD5 | LY6/PLAUR domain containing 5 | 159871 | 104783 | 167637 | |
| 19 | LYL1 | LYL1, basic helix-loop-helix family member | 104903 | 008441 | 104907 | 22, 41, 38 |
| 19 | KDM4B | lysine demethylase 4B | 127663 | 276043 | 105426 | |
| 19 | KMT2B | lysine methyltransferase 2B | 272333 | 011590 | 126246 | |
| 19 | KMT5C | lysine methyltransferase 5C | 133247 | 180061 | 160471 | 18, 44, 9 |
| 19 | LPAR2 | lysophosphatidic acid receptor 2 | 064547 | 105717 | 089639 | 29, 38, 12 |
| 19 | MFSD12 | major facilitator superfamily domain containing 12 | 161091 | 105325 | 183397 | 35, 9, 20 |
| 19 | MAN2B1 | mannosidase alpha class 2B member 1 | 104774 | 269242 | 269590 | 39, 36, 10 |
| 19 | MKNK2 | MAP kinase interacting serine/threonine kinase 2 | 099875 | 133243 | 172081 | 3, 21, 41 |
| 19 | MCEMP1 | mast cell expressed membrane protein 1 | 183019 | 104918 | 269711 | 20, 5, 2 |
| 19 | MAU2 | MAU2 sister chromatid cohesion factor | 129933 | 105705 | 167491 | 18, 39, 37 |
| 19 | MED16 | mediator complex subunit 16 | 175221 | 197766 | 198858 | 29, 34, 27 |
| 19 | MED25 | mediator complex subunit 25 | 104973 | 010361 | 104960 | |
| 19 | MED26 | mediator complex subunit 26 | 105085 | 127526 | 268790 | 38, 9, 27 |
| 19 | MED29 | mediator complex subunit 29 | 063322 | 006712 | 128016 | 42, 8, 36 |
| 19 | MAMSTR | MEF2 activating motif and SAP domain containing | 176909 | 176920 | 105538 | 2, 45, 25 |
| | | transcriptional regulator | | | | |
| 19 | MATK | megakaryocyte-associated tyrosine kinase | 007264 | 173976 | 105278 | 5, 14, 36 |
| 19 | MEIS3 | Meis homeobox 3 | 105419 | 134815 | 118160 | 37, 18, 9 |
| 19 | MUM1 | melanoma associated antigen (mutated) 1 | 160953 | 099617 | 115286 | 26, 27, 40 |
| 19 | MIA | melanoma inhibitory activity | 261857 | 077312 | 167578 | 15, 11, 35 |
| 19 | MARCH2 | membrane associated ring-CH-type finger 2 | 099785 | 185236 | 099783 | 21, 40, 31 |
| 19 | MBOAT7 | membrane bound O-acyltransferase domain containing 7 | 125505 | 167608 | 170892 | 3, 27, 5 |
| 19 | MBD3 | methyl-CpG binding domain protein 3 | 071655 | 181588 | 267059 | 6, |
| 19 | MBD3L1 | methyl-CpG binding domain protein 3 like 1 | 170948 | 167785 | 181143 | 38, |
| 19 | MBD3L2 | methyl-CpG binding domain protein 3 like 2 | 230522 | 205718 | 182315 | |
| 19 | MBD3L3 | methyl-CpG binding domain protein 3 like 3 | 182315 | 230522 | 130544 | |
| 19 | MBD3L4 | methyl-CpG binding domain protein 3 like 4 | 205718 | 237247 | 230522 | |
| 19 | MBD3L5 | methyl-CpG binding domain protein 3 like 5 | 237247 | 196589 | 205718 | |
| 19 | MRI1 | methylthioribose-1-phosphate isomerase 1 | 037757 | 104957 | 104979 | 9, 33, 31 |
| 19 | MEX3D | mex-3 RNA binding family member D | 181588 | 185988 | 071655 | 31, 27, 3 |
| 19 | MIA-RAB4B | MIA-RAB4B readthrough (NMD candidate) | 268975 | 077312 | 167578 | |
| 19 | MARK4 | microtubule affinity regulating kinase 4 | 007047 | 142252 | 104866 | 34, 21, 25 |
| 19 | MAP1S | microtubule associated protein 1S | 130479 | 130477 | 130475 | 38, 39, 8 |
| 19 | MAST1 | microtubule associated serine/threonine kinase 1 | 105613 | 132026 | 105612 | 26, 31, 14 |
| 19 | MAST3 | microtubule associated serine/threonine kinase 3 | 099308 | 096996 | 105647 | 22, 26, 21 |
| 19 | MIDN | midnolin | 167470 | 099624 | 099622 | 34, 12, 22 |
| 19 | MIER2 | MIER family member 2 | 105556 | 141934 | 105549 | 26, 9, 31 |
| 19 | MISP3 | MISP family member 3 | 141854 | 187867 | 188032 | |
| 19 | MRPL34 | mitochondrial ribosomal protein L34 | 130312 | 127220 | 130311 | |
| 19 | MRPL4 | mitochondrial ribosomal protein L4 | 105364 | 267534 | 090339 | 21, 3, 35 |
| 19 | MRPL54 | mitochondrial ribosomal protein L54 | 183617 | 011132 | 173976 | 3, 29, 7 |
| 19 | MRPS12 | mitochondrial ribosomal protein S12 | 128626 | 269547 | 269190 | 28, 41, 3 |
| 19 | MAP2K2 | mitogen-activated protein kinase kinase 2 | 126934 | 178951 | 060566 | 38, 33, |
| 19 | MAP2K7 | mitogen-activated protein kinase kinase 7 | 076984 | 171017 | 260001 | 22, 27, 14 |
| 19 | MAP3K10 | mitogen-activated protein kinase kinase kinase 10 | 130758 | 197782 | 174521 | 37, 9, 2 |
| 19 | MAP4K1 | mitogen-activated protein kinase kinase kinase kinase 1 | 104814 | 196218 | 178982 | 41, 22, 21 |
| 19 | MISP | mitotic spindle positioning | 099812 | 099864 | 011304 | 16, 9, 37 |
| 19 | MLLT1 | MLLT1, super elongation complex subunit | 130382 | 130377 | 167769 | 34, 32, 22 |
| 19 | MOB3A | MOB kinase activator 3A | 172081 | 099875 | 099840 | 39, 36, 22 |
| 19 | MPND | MPN domain containing | 008382 | 178078 | 141985 | 24, 27, 40 |
| 19 | MPV17L2 | MPV17 mitochondrial inner membrane protein like 2 | 254858 | 216490 | 105649 | 2, 27, 36 |
| 19 | MUC16 | mucin 16, cell surface associated | 181143 | 170948 | 170929 | 42, 23, 1 |
| 19 | MCOLN1 | mucolipin 1 | 090674 | 198816 | 268614 | 26, 5, 14 |
| 19 | MADCAM1 | mucosal vascular addressin cell adhesion molecule 1 | 099866 | 181781 | 141933 | 40, 36, 15 |
| 19 | MEGF8 | multiple EGF like domains 8 | 105429 | 167619 | 105427 | 21, 6, 4 |
| 19 | MVB12A | muItivesicular body subunit 12A | 141971 | 130303 | 188051 | 41, 9, 21 |
| 19 | MYPOP | Myb related transcription factor, partner of profilin | 176182 | 170604 | 188425 | 26, 21, 2 |
| 19 | MAG | myelin associated glycoprotein | 105695 | 105697 | 012124 | 8, 17, 26 |
| 19 | MYADM | myeloid associated differentiation marker | 179820 | 142405 | 126583 | 5, 40, 45 |
| 19 | MYDGF | myeloid derived growth factor | 074842 | 185361 | 142002 | |
| 19 | MZF1 | myeloid zinc finger 1 | 099326 | 130725 | 243772 | 29, 22, 23 |
| 19 | MEF2B | myocyte enhancer factor 2B | 213999 | 064489 | 254901 | |
| 19 | MYBPC2 | myosin binding protein C, fast type | 086967 | 269404 | 142530 | 25, 29, 40 |
| 19 | MYH14 | myosin heavy chain 14 | 105357 | 161652 | 131398 | 16, 40, 18 |
| 19 | MYO1F | myosin IF | 142347 | 133250 | 142303 | 36, 5, 7 |
| 19 | MYO9B | myosin IXB | 099331 | 131351 | 053501 | 22, 41, 21 |
| 19 | NAT14 | N-acetyltransferase 14 (putative) | 090971 | 197483 | 179954 | 28, 15, 8 |
| 19 | NWD1 | NACHT and WD repeat domain containing 1 | 188039 | 072954 | 127511 | 30, 18, 33 |
| 19 | NDUFS7 | NADH: ubiquinone oxidoreductase core subunit S7 | 115286 | 160953 | 130005 | 34, 25, 39 |
| 19 | NDUFA11 | NADH: ubiquinone oxidoreductase subunit A11 | 174886 | 267740 | 267314 | 34, 29, 25 |
| 19 | NDUFA13 | NADH: ubiquinone oxidoreductase subunit A13 | 186010 | 178093 | 258674 | 29, 43, 25 |
| 19 | NDUFA3 | NADH: ubiquinone oxidoreductase subunit A3 | 170906 | 170909 | 105619 | 7, 25, 33 |
| 19 | NDUFA7 | NADH: ubiquinone oxidoreductase subunit A7 | 267855 | 167774 | 233927 | 7, 26, 25 |
| 19 | NDUFB7 | NADH: ubiquinone oxidoreductase subunit B7 | 099795 | 099797 | 187912 | 31, 27, 3 |
| 19 | NANOS2 | nanos C2HC-type zinc finger 2 | 188425 | 176182 | 104967 | 38, |
| 19 | NANOS3 | nanos C2HC-type zinc finger 3 | 187556 | 132003 | 132016 | 28, 24, 20 |
| 19 | NAPSA | napsin A aspartic peptidase | 131400 | 131408 | 062822 | 20, 10, 33 |
| 19 | NCR1 | natural cytotoxicity triggering receptor 1 | 189430 | 186431 | 167634 | 36, 5, 38 |
| 19 | NKG7 | natural killer cell granule protein 7 | 105374 | 160318 | 105370 | 5, 30, 31 |
| 19 | NECTIN2 | nectin cell adhesion molecule 2 | 130202 | 187244 | 130204 | |
| 19 | NTN5 | netrin 5 | 142233 | 063180 | 176920 | 25, 21, 40 |
| 19 | NCAN | neurocan | 130287 | 184162 | 187664 | 8, 45, 24 |
| 19 | NPAS1 | neuronal PAS domain protein 1 | 130751 | 160007 | 130748 | 34, 41, 35 |
| 19 | NTF4 | neurotrophin 4 | 225950 | 283663 | 104848 | |
| 19 | NRTN | neurturin | 171119 | 141994 | 156413 | 14, 28, 25 |
| 19 | NFKBIB | NFKB inhibitor beta | 104825 | 068903 | 262484 | 26, 3, 28 |
| 19 | NFKBID | NFKB inhibitor delta | 167604 | 105290 | 126264 | 40, 41, 5 |
| 19 | NCLN | nicalin | 125912 | 125910 | 161082 | 3, 16, 26 |
| 19 | NMRK2 | nicotinamide riboside kinase 2 | 077009 | 167654 | 167657 | 15, 25, 14 |
| 19 | NOSIP | nitric oxide synthase interacting protein | 142546 | 142552 | 126460 | 29, 34, 19 |
| 19 | NLRP11 | NLR family pyrin domain containing 11 | 179873 | 229292 | 160505 | 41, 21, 37 |
| 19 | NLRP12 | NLR family pyrin domain containing 12 | 142405 | 204595 | 179820 | 5, 33, 7 |
| 19 | NLRP13 | NLR family pyrin domain containing 13 | 173572 | 160505 | 179709 | |
| 19 | NLRP2 | NLR family pyrin domain containing 2 | 022556 | 167634 | 088053 | 32, 38, 4 |
| 19 | NLRP4 | NLR family pyrin domain containing 4 | 160505 | 179873 | 173572 | 41, 38, 21 |
| 19 | NLRP5 | NLR family pyrin domain containing 5 | 171487 | 179709 | 142409 | 6, |
| 19 | NLRP7 | NLR family pyrin domain containing 7 | 167634 | 189430 | 022556 | 21, 30, 38 |
| 19 | NLRP8 | NLR family pyrin domain containing 8 | 179709 | 173572 | 171487 | |
| 19 | NLRP9 | NLR family pyrin domain containing 9 | 185792 | 063245 | 223638 | 22, 33, 44 |
| 19 | NCCRP1 | non-specific cytotoxic cell receptor protein 1 homolog | 188505 | 130669 | 179751 | 35, 36, 30 |
| | | (zebrafish) | | | | |
| 19 | NOTCH3 | notch 3 | 074181 | 105135 | 105131 | 27, 12, 26 |
| 19 | NOVA2 | NOVA alternative splicing regulator 2 | 104967 | 188425 | 104983 | 29, 14, 28 |
| 19 | NPHS1 | NPHS1, nephrin | 161270 | 250799 | 126259 | 8, |
| 19 | NAPA | NSF attachment protein alpha | 105402 | 118162 | 118156 | 34, 28, 2 |
| 19 | NKPD1 | NTPase, KAP family P-loop domain containing 1 | 179846 | 104866 | 007255 | 15, 22, 27 |
| 19 | NFIC | nuclear factor I C | 141905 | 161082 | 095932 | 45, 40, 1 |
| 19 | NFIX | nuclear factor I X | 008441 | 179284 | 104903 | 6, 40, 33 |
| 19 | NR2C2AP | nuclear receptor 2C2 associated protein | 184162 | 064490 | 130287 | 30, 13, 16 |
| 19 | NR1H2 | nuclear receptor subfamily 1 group H member 2 | 131408 | 131398 | 131400 | 7, 25, 41 |
| 19 | NR2F6 | nuclear receptor subfamily 2 group F member 6 | 160113 | 099330 | 269095 | 27, 34, 29 |
| 19 | NUCB1 | nucleobindin 1 | 104805 | 104804 | 104808 | 36, 45, 2 |
| 19 | NUP62 | nucleoporin 62 | 213024 | 104951 | 269179 | 38, |
| 19 | NXNL1 | nucleoredoxin-like 1 | 171773 | 269035 | 130304 | 14, 38, |
| 19 | NACC1 | nucleus accumbens associated 1 | 160877 | 104907 | 104915 | 26, 9, 12 |
| 19 | NUDT19 | nudix hydrolase 19 | 213965 | 186326 | 173809 | 37, 6, 30 |
| 19 | NUMBL | NUMB like, endocytic adaptor protein | 105245 | 279183 | 123815 | 28, 26, 4 |
| 19 | OCEL1 | occludin/ELL domain containing 1 | 099330 | 053501 | 160113 | 10, 30, 14 |
| 19 | OLFM2 | olfactomedin 2 | 105088 | 127445 | 080573 | 26, 14, 35 |
| 19 | OR1I1 | olfactory receptor family 1 subfamily I member 1 | 094661 | 105141 | 105137 | |
| 19 | OR1M1 | olfactory receptor family 1 subfamily M member 1 | 170929 | 181143 | 170923 | |
| 19 | OR10H1 | olfactory receptor family 10 subfamily H member 1 | 186723 | 172519 | 186115 | |
| 19 | OR10H2 | olfactory receptor family 10 subfamily H member 2 | 171942 | 186204 | 171936 | 10, |
| 19 | OR10H3 | olfactory receptor family 10 subfamily H member 3 | 171936 | 171942 | 172519 | |
| 19 | OR10H4 | olfactory receptor family 10 subfamily H member 4 | 176231 | 171903 | 167460 | |
| 19 | OR10H5 | olfactory receptor family 10 subfamily H member 5 | 172519 | 171936 | 186723 | |
| 19 | OR2Z1 | olfactory receptor family 2 subfamily Z member 1 | 181733 | 181786 | 167785 | |
| 19 | OR4F17 | olfactory receptor family 4 subfamily F member 17 | 176695 | 243772 | 141934 | |
| 19 | OR7A10 | olfactory receptor family 7 subfamily A member 10 | 127515 | 127530 | 185385 | |
| 19 | OR7A17 | olfactory receptor family 7 subfamily A member 17 | 185385 | 127515 | 127529 | 23, |
| 19 | OR7A5 | olfactory receptor family 7 subfamily A member 5 | 188269 | 127507 | 127530 | 24, 38, 8 |
| 19 | OR7C1 | olfactory receptor family 7 subfamily C member 1 | 127530 | 188269 | 127515 | 38, |
| 19 | OR7C2 | olfactory receptor family 7 subfamily C member 2 | 127529 | 185385 | 105143 | |
| 19 | OR7D2 | olfactory receptor family 7 subfamily D member 2 | 188000 | 130803 | 174667 | 5, |
| 19 | OR7D4 | olfactory receptor family 7 subfamily D member 4 | 174667 | 188000 | 237521 | 40, |
| 19 | OR7E24 | olfactory receptor family 7 subfamily E member 24 | 237521 | 174667 | 196110 | 38, |
| 19 | OR7G1 | olfactory receptor family 7 subfamily G member 1 | 161807 | 170923 | 170920 | |
| 19 | OR7G2 | olfactory receptor family 7 subfamily G member 2 | 170923 | 170929 | 161807 | |
| 19 | OR7G3 | olfactory receptor family 7 subfamily G member 3 | 170920 | 161807 | 130803 | |
| 19 | ONECUT3 | one cut homeobox 3 | 205922 | 071564 | 130270 | 14, |
| 19 | OPA3 | OPA3, outer mitochondrial membrane lipid metabolism regulator | 125741 | 125753 | 177464 | 18, 37, 32 |
| 19 | OAZ1 | ornithine decarboxylase antizyme 1 | 104904 | 167476 | 273734 | 41, 34, 21 |
| 19 | OSCAR | osteoclast associated, immunoglobulin-like receptor | 170909 | 248385 | 170906 | 5, 7, 19 |
| 19 | ODF3L2 | outer dense fiber of sperm tails 3 like 2 | 181781 | 129946 | 099866 | 38, 16, 8 |
| 19 | OVOL3 | ovo like zinc finger 3 | 105261 | 075702 | 105258 | 27, 10, 38 |
| 19 | PAK4 | p21 (RAC1) activated kinase 4 | 130669 | 183760 | 188505 | 33, 28, 34 |
| 19 | PAF1 | PAF1 homolog, Paf1/RNA polymerase II complex component | 006712 | 179134 | 063322 | 28, 40, 18 |
| 19 | PALM | paralemmin | 099864 | 185198 | 099812 | 34, 26, 17 |
| 19 | PALM3 | paralemmin 3 | 187867 | 104998 | 141854 | 4, 14, 15 |
| 19 | PNMAL1 | paraneoplastic Ma antigen family like 1 | 182013 | 277531 | 230510 | 40, 29, 26 |
| 19 | PNMAL2 | paraneoplastic Ma antigen family like 2 | 204851 | 230510 | 160014 | 26, 2, 14 |
| 19 | PTH2 | parathyroid hormone 2 | 142538 | 161609 | 261949 | |
| 19 | PNPLA6 | patatin like phospholipase domain containing 6 | 032444 | 268614 | 076826 | 7, 38, 8 |
| 19 | PEG3 | paternally expressed 3 | 198300 | 269699 | 131864 | 31, 2, 32 |
| 19 | PBX4 | PBX homeobox 4 | 105717 | 160161 | 064547 | 39, 3, 20 |
| 19 | PEPD | peptidase D | 124299 | 153879 | 124302 | 17, 2, 37 |
| 19 | PGLYRP1 | peptidoglycan recognition protein 1 | 008438 | 104983 | 204869 | 7, 5, 10 |
| 19 | PGLYRP2 | peptidoglycan recognition protein 2 | 161031 | 105122 | 171954 | 38, 19, 23 |
| 19 | PIN1 | peptidylprolyl cis/trans isomerase, NIMA-interacting 1 | 127445 | 198258 | 105088 | |
| 19 | PRX | periaxin | 105227 | 160396 | 197019 | 26, 14, 12 |
| 19 | PLIN3 | perilipin 3 | 105355 | 127666 | 205784 | 21, 41, 35 |
| 19 | PLIN4 | perilipin 4 | 167676 | 167674 | 214456 | 1, 23, 26 |
| 19 | PLIN5 | perilipin 5 | 214456 | 167676 | 267385 | 29, 1, 25 |
| 19 | PRDX2 | peroxiredoxin 2 | 167815 | 171223 | 104889 | 5, 24, 2 |
| 19 | PEX11G | peroxisomal biogenesis factor 11 gamma | 104883 | 104880 | 198723 | 37, 16, 38 |
| 19 | PSPN | persephin | 125650 | 125652 | 125651 | 20, |
| 19 | PET100 | PET100 homolog | 229833 | 076924 | 268400 | 43, 11, 7 |
| 19 | PPAN | peter pan homolog (Drosophila) | 130810 | 243207 | 244165 | |
| 19 | FARSA | phenylalanyl-tRNA synthetase alpha subunit | 179115 | 161860 | 179218 | 43, 2, 21 |
| 19 | PIP5K1C | phosphatidylinositol-4-phosphate 5-kinase type 1 gamma | 186111 | 105298 | 105289 | 26, 34, 14 |
| 19 | PDE4A | phosphodiesterase 4A | 065989 | 105401 | 079999 | 34, 18, 27 |
| 19 | PDE4C | phosphodiesterase 4C | 105650 | 105649 | 130518 | 27, 20, 16 |
| 19 | PIK3R2 | phosphoinositide-3-kinase regulatory subunit 2 | 105647 | 099308 | 268173 | 34, 27, 18 |
| 19 | PLA2G4C | phospholipase A2 group IVC | 105499 | 105507 | 105486 | 36, 45, 1 |
| 19 | PINLYP | phospholipase A2 inhibitor and LY6/PLAUR domain containing | 234465 | 268361 | 167378 | 29, 17, 20 |
| 19 | PLD3 | phospholipase D family member 3 | 105223 | 160392 | 160396 | 26, 27, 31 |
| 19 | PLPP2 | phospholipid phosphatase 2 | 141934 | 176695 | 105556 | 16, 31, 44 |
| 19 | PLPPR2 | phospholipid phosphatase related 2 | 105520 | 183401 | 173928 | |
| 19 | PLPPR3 | phospholipid phosphatase related 3 | 129951 | 011304 | 172232 | 7, 12, 14 |
| 19 | PIH1D1 | PIH1 domain containing 1 | 104872 | 104888 | 161618 | |
| 19 | PLVAP | plasmalemma vesicle associated protein | 130300 | 130299 | 269720 | 24, 40, 36 |
| 19 | PLAUR | plasminogen activator, urokinase receptor | 011422 | 105767 | 124449 | 20, 27, 5 |
| 19 | PAFAH1B3 | platelet activating factor acetylhydrolase 1b catalytic | 079462 | 079432 | 188368 | 31, 27, 15 |
| | | subunit 3 | | | | |
| 19 | PLEKHF1 | pleckstrin homology and FYVE domain containing 1 | 166289 | 105171 | 131943 | 27, 34, 32 |
| 19 | PLEKHG2 | pleckstrin homology and RhoGEF domain containing G2 | 090924 | 128016 | 105193 | 28, 22, 20 |
| 19 | PLEKHA4 | pleckstrin homology domain containing A4 | 105559 | 087076 | 087074 | 35, 44, 31 |
| 19 | PLEKHJ1 | pleckstrin homology domain containing J1 | 104886 | 104885 | 104897 | 41, 12, 34 |
| 19 | PHLDB3 | pleckstrin homology like domain family B member 3 | 176531 | 124466 | 105755 | 39, 27, 15 |
| 19 | PRAM1 | PML-RARA regulated adaptor molecule 1 | 133246 | 099783 | 133250 | 5, 39, 40 |
| 19 | PODNL1 | podocan like 1 | 132000 | 132024 | 132017 | 34, 10, 6 |
| 19 | PVR | poliovirus receptor | 073008 | 216588 | 186567 | 45, 17, 30 |
| 19 | PLK5 | polo like kinase 5 | 185988 | 185761 | 181588 | 19, 8, 38 |
| 19 | PNKP | polynucleotide kinase 3′-phosphatase | 039650 | 104960 | 204673 | 9, 27, 20 |
| 19 | PTBP1 | polypyrimidine tract binding protein 1 | 011304 | 099812 | 129951 | 32, 12, 22 |
| 19 | POP4 | POP4 homolog, ribonuclease P/MRP subunit | 105171 | 187135 | 166289 | 27, 3, 21 |
| 19 | KCNN1 | potassium calcium-activated channel subfamily N member 1 | 105642 | 007080 | 105643 | 45, 14, 8 |
| 19 | KCNN4 | potassium calcium-activated channel subfamily N member 4 | 104783 | 105771 | 159871 | 3, 32, 33 |
| 19 | KCTD15 | potassium channel tetramerization domain containing 15 | 153885 | 124302 | 257103 | 45, 29, 15 |
| 19 | KCNK6 | potassium two pore domain channel subfamily K member 6 | 099337 | 167645 | 099338 | 24, 36, 34 |
| 19 | KCNA7 | potassium voltage-gated channel subfamily A member 7 | 104848 | 225950 | 104852 | 25, |
| 19 | KCNC3 | potassium voltage-gated channel subfamily C member 3 | 131398 | 105357 | 131408 | 29, 18, 12 |
| 19 | KCNJ14 | potassium voltage-gated channel subfamily J member 14 | 182324 | 105447 | 268465 | 27, 14, 5 |
| 19 | POU2F2 | POU class 2 homeobox 2 | 028277 | 105732 | 160570 | 22, 41, 23 |
| 19 | PPAN-P2RY11 | PPAN-P2RY11 readthrough | 243207 | 130812 | 130810 | |
| 19 | PPP5D1 | PPP5 tetratricopeptide repeat domain containing 1 | 230510 | 182013 | 204851 | |
| 19 | PSG1 | pregnancy specific beta-1-glycoprotein 1 | 231924 | 124467 | 170848 | 32, 18, 45 |
| 19 | PSG11 | pregnancy specific beta-1-glycoprotein 11 | 243130 | 221878 | 242221 | 32, 45, 10 |
| 19 | PSG2 | pregnancy specific beta-1-glycoprotein 2 | 242221 | 243130 | 204941 | 32, 10, 6 |
| 19 | PSG3 | pregnancy specific beta-1-glycoprotein 3 | 221826 | 124469 | 124467 | 32, 45, 38 |
| 19 | PSG4 | pregnancy specific beta-1-glycoprotein 4 | 243137 | 204941 | 183668 | 32, 29, 35 |
| 19 | PSG5 | pregnancy specific beta-1-glycoprotein 5 | 204941 | 242221 | 243137 | 32, 35, 4 |
| 19 | PSG6 | pregnancy specific beta-1-glycoprotein 6 | 170848 | 231924 | 221878 | 32, 45, 15 |
| 19 | PSG7 | pregnancy specific beta-1-glycoprotein 7 (gene/pseudogene) | 221878 | 170848 | 243130 | 32, 5, |
| 19 | PSG8 | pregnancy specific beta-1-glycoprotein 8 | 124467 | 221826 | 231924 | 32, 37, 40 |
| 19 | PSG9 | pregnancy specific beta-1-glycoprotein 9 | 183668 | 243137 | 204936 | 32, 45, 36 |
| 19 | PRPF31 | pre-mRNA processing factor 31 | 105618 | 105619 | 088038 | 27, 34, 14 |
| 19 | PSENEN | presenilin enhancer gamma-secretase subunit | 205155 | 161265 | 188223 | 30, 7, 29 |
| 19 | PDCD2L | programmed cell death 2 like | 126249 | 266953 | 126261 | 41, 3, 38 |
| 19 | PDCD5 | programmed cell death 5 | 105185 | 178904 | 105186 | 10, 33, 44 |
| 19 | PROSER3 | proline and serine rich 3 | 167595 | 004776 | 004777 | 18, 38, 37 |
| 19 | PRODH2 | proline dehydrogenase 2 | 250799 | 004777 | 161270 | 17, 19, |
| 19 | PRR12 | proline rich 12 | 126464 | 126460 | 126458 | 14, 4, 31 |
| 19 | PRR19 | proline rich 19 | 188368 | 079462 | 167619 | 41, 22, 38 |
| 19 | PRR22 | proline rich 22 | 212123 | 174898 | 267157 | 37, 38, 6 |
| 19 | PRR36 | proline rich 36 | 183248 | 142459 | 171017 | 26, 14, 8 |
| 19 | PRRG2 | proline rich and Gla domain 2 | 126460 | 142546 | 126464 | 3, 27, 40 |
| 19 | PCSK4 | proprotein convertase subtilisin/kexin type 4 | 115257 | 119559 | 115255 | 38, 39, 24 |
| 19 | PTGER1 | prostaglandin E receptor 1 | 160951 | 123143 | 123159 | 17, 28, 36 |
| 19 | PTGIR | prostaglandin I2 (prostacyclin) receptor (IP) | 160013 | 160014 | 167414 | 7, 1, 5 |
| 19 | PTOV1 | prostate tumor overexpressed 1 | 104960 | 104973 | 039650 | 20, 14, 27 |
| 19 | PRSS57 | protease, serine 57 | 185198 | 070404 | 099864 | 7, 5, 38 |
| 19 | PSMC4 | proteasome 26S subunit, ATPase 4 | 013275 | 275395 | 187187 | 27, 28, 21 |
| 19 | PSMD8 | proteasome 26S subunit, non-ATPase 8 | 099341 | 099338 | 179168 | 5, 29, 16 |
| 19 | PRMT1 | protein arginine methyltransferase 1 | 126457 | 126453 | 224420 | 21, 3, 35 |
| 19 | PIAS4 | protein inhibitor of activated STAT 4 | 105229 | 167658 | 178951 | 38, 26, 12 |
| 19 | PRKCG | protein kinase C gamma | 126583 | 179820 | 105605 | 8, 39, 17 |
| 19 | PRKCSH | protein kinase C substrate 80K-H | 130175 | 198003 | 196361 | 39, 36, 45 |
| 19 | PRKACA | protein kinase cAMP-activated catalytic subunit alpha | 072062 | 141858 | 105011 | 26, 31, 36 |
| 19 | PRKD2 | protein kinase D2 | 105287 | 197380 | 090372 | 39, 36, 42 |
| 19 | PKN1 | protein kinase N1 | 123143 | 123136 | 160951 | 21, 41, 34 |
| 19 | PPP1R14A | protein phosphatase 1 regulatory inhibitor subunit 14A | 167641 | 167642 | 267748 | 43, 15, 28 |
| 19 | PPP1R12C | protein phosphatase 1 regulatory subunit 12C | 125503 | 131037 | 105048 | 29, 34, 26 |
| 19 | PPP1R13L | protein phosphatase 1 regulatory subunit 13 like | 104881 | 104884 | 117877 | 37, 23, 44 |
| 19 | PPP1R15A | protein phosphatase 1 regulatory subunit 15A | 087074 | 105559 | 104804 | 13, 41, 4 |
| 19 | PPP1R37 | protein phosphatase 1 regulatory subunit 37 | 104866 | 007047 | 179846 | |
| 19 | PPP2R1A | protein phosphatase 2 scaffold subunit Aalpha | 105568 | 196267 | 196214 | 2, 36, 8 |
| 19 | PPP5C | protein phosphatase 5 catalytic subunit | 011485 | 124440 | 169515 | 4, 35, 43 |
| 19 | PPP6R1 | protein phosphatase 6 regulatory subunit 1 | 105063 | 180089 | 133265 | 41, 21, 35 |
| 19 | PPM1N | protein phosphatase, Mg2+/Mn2+ dependent 1N (putative) | 213889 | 125744 | 125753 | 10, 34, 22 |
| 19 | PTPRH | protein tyrosine phosphatase, receptor type H | 080031 | 129990 | 180089 | 36, 42, 16 |
| 19 | PTPRS | protein tyrosine phosphatase, receptor type S | 105426 | 127663 | 283229 | 18, 33, 6 |
| 19 | PRTN3 | proteinase 3 | 196415 | 172232 | 197561 | 5, 7, |
| 19 | PPFIA3 | PTPRF interacting protein alpha 3 | 177380 | 221916 | 130528 | 26, 6, 3 |
| 19 | P2RY11 | purinergic receptor P2Y11 | 244165 | 130810 | 130811 | 21, 5, 35 |
| 19 | PCP2 | Purkinje cell protein 2 | 174788 | 268400 | 076944 | 14, 38, 40 |
| 19 | PGPEP1 | pyroglutamyl-peptidase I | 130517 | 130520 | 130513 | 17, 34, 1 |
| 19 | QTRT1 | queuine tRNA-ribosyltransferase catalytic subunit 1 | 213339 | 129351 | 079805 | 26, 21, 3 |
| 19 | R3HDM4 | R3H domain containing 4 | 198858 | 175221 | 116014 | 41, 34, 5 |
| 19 | RABAC1 | Rab acceptor 1 | 105404 | 076928 | 105409 | 28, 21, 31 |
| 19 | RAB11B | RAB11B, member RAS oncogene family | 185236 | 167772 | 099785 | 36, 34, 21 |
| 19 | RAB3A | RAB3A, member RAS oncogene family | 105649 | 254858 | 105650 | 8, 14, 26 |
| 19 | RAB3D | RAB3D, member RAS oncogene family | 105514 | 130167 | 105518 | 1, 27, 29 |
| 19 | RAB4B | RAB4B, member RAS oncogene family | 167578 | 268975 | 171570 | |
| 19 | RAB4B-EGLN2 | RAB4B-EGLN2 readthrough (NMD candidate) | 171570 | 167578 | 269858 | |
| 19 | RAB8A | RAB8A, member RAS oncogene family | 167461 | 167460 | 196684 | 21, 13, 35 |
| 19 | RAD23A | RAD23 homolog A, nucleotide excision repair protein | 179262 | 179218 | 179271 | 3, 34, 6 |
| 19 | RSPH6A | radial spoke head 6 homolog A | 104941 | 185800 | 125755 | 38, |
| 19 | RGL3 | ral guanine nucleotide dissociation stimulator like 3 | 205517 | 187266 | 198003 | 40, 29, 36 |
| 19 | RANBP3 | RAN binding protein 3 | 031823 | 105519 | 087903 | 39, 3, 38 |
| 19 | RINL | Ras and Rab interactor like | 187994 | 104824 | 068903 | 41, 26, 28 |
| 19 | RASGRP4 | RAS guanyl releasing protein 4 | 171777 | 130244 | 196218 | 7, 5, 36 |
| 19 | RASIP1 | Ras interacting protein 1 | 105538 | 176909 | 182264 | 9, 43, 27 |
| 19 | RASAL3 | RAS protein activator like 3 | 105122 | 011451 | 161031 | 5, 41, 1 |
| 19 | REEP6 | receptor accessory protein 6 | 115255 | 115257 | 185761 | 38, 7, 27 |
| 19 | RGS9BP | regulator of G-protein signaling 9 binding protein | 186326 | 105186 | 213965 | 15, 14, 33 |
| 19 | RFXANK | regulatory factor X associated ankyrin containing protein | 064490 | 254901 | 184162 | 21, 34, 22 |
| 19 | RFX1 | regulatory factor X1 | 132005 | 132017 | 171136 | |
| 19 | RFX2 | regulatory factor X2 | 087903 | 031823 | 130377 | |
| 19 | RRAS | related RAS viral (r-ras) oncogene homolog | 126458 | 126464 | 126461 | 33, 21, 25 |
| 19 | RLN3 | relaxin 3 | 171136 | 132005 | 104998 | 38, |
| 19 | RELB | RELB proto-oncogene, NF-kB subunit | 104856 | 104853 | 104859 | 21, 36, 22 |
| 19 | RETN | resistin | 104918 | 076944 | 183019 | 7, 5, 16 |
| 19 | RFPL4A | ret finger protein like 4A | 223638 | 185792 | 229292 | |
| 19 | RFPL4AL1 | ret finger protein like 4A like 1 | 229292 | 223638 | 179873 | |
| 19 | RTBDN | retbindin | 132026 | 104889 | 105613 | 14, 39, 6 |
| 19 | RCN3 | reticulocalbin 3 | 142552 | 104870 | 142546 | 6, 15, 27 |
| 19 | RTN2 | reticulon 2 | 125744 | 125740 | 213889 | 25, 26, 29 |
| 19 | RAX2 | retina and anterior neural fold homeobox 2 | 173976 | 183617 | 007264 | 14, |
| 19 | RDH13 | retinol dehydrogenase 13 | 160439 | 088053 | 131037 | |
| 19 | RDH8 | retinol dehydrogenase 8 (all-trans) | 080511 | 080573 | 130813 | 14, 35, 25 |
| 19 | ARHGAP33 | Rho GTPase activating protein 33 | 004777 | 167595 | 250799 | 22, 8, 27 |
| 19 | ARHGAP35 | Rho GTPase activating protein 35 | 160007 | 042753 | 130751 | 22, 26, 40 |
| 19 | ARHGAP45 | Rho GTPase activating protein 45 | 180448 | 064687 | 099817 | 39, 21, 41 |
| 19 | ARHGEF1 | Rho guanine nucleotide exchange factor 1 | 076928 | 105369 | 105404 | |
| 19 | ARHGEF18 | Rho/Rac guanine nucleotide exchange factor 18 | 104880 | 268861 | 104883 | 21, 22, 27 |
| 19 | RHPN2 | rhophilin Rho GTPase binding protein 2 | 131941 | 131944 | 076650 | 42, 37, 27 |
| 19 | RNASEH2A | ribonuclease H2 subunit A | 104889 | 167815 | 132026 | 21, 41, 9 |
| 19 | RAVER1 | ribonucleoprotein, PTB binding 1 | 161847 | 267303 | 076662 | 21, 9, 26 |
| 19 | RPL13A | ribosomal protein L13a | 142541 | 090554 | 142534 | 41, 43, 21 |
| 19 | RPL18 | ribosomal protein L18 | 063177 | 177202 | 063176 | 41, 21, 43 |
| 19 | RPL18A | ribosomal protein L18a | 105640 | 105639 | 105641 | 41, 12, 21 |
| 19 | RPL28 | ribosomal protein L28 | 108107 | 233493 | 108106 | 41, 27, 21 |
| 19 | RPL36 | ribosomal protein L36 | 130255 | 160633 | 174917 | 31, 43, 7 |
| 19 | RPS11 | ribosomal protein S11 | 142534 | 142541 | 104870 | 18, 43, 7 |
| 19 | RPS15 | ribosomal protein S15 | 115268 | 071626 | 115266 | 43, 41, 31 |
| 19 | RPS16 | ribosomal protein S16 | 105193 | 090924 | 196235 | 28, 43, 27 |
| 19 | RPS19 | ribosomal protein S19 | 105372 | 142025 | 105369 | 30, 43, 33 |
| 19 | RPS28 | ribosomal protein S28 | 233927 | 267855 | 186994 | |
| 19 | RPS5 | ribosomal protein S5 | 083845 | 152475 | 269855 | 41, 43, 21 |
| 19 | RPS9 | ribosomal protein S9 | 170889 | 170892 | 204577 | 7, 41, 27 |
| 19 | RNF126 | ring finger protein 126 | 070423 | 070388 | 070404 | 41, 3, 9 |
| 19 | RNF225 | ring finger protein 225 | 269855 | 083845 | 171574 | 38, |
| 19 | RBM42 | RNA binding motif protein 42 | 126254 | 249115 | 105672 | 21, 35, 27 |
| 19 | REXO1 | RNA exonuclease 1 homolog | 079313 | 130270 | 129911 | 26, 18, 4 |
| 19 | POLR2E | RNA polymerase II subunit E | 099817 | 180448 | 167468 | 41, 1, 21 |
| 19 | POLR2I | RNA polymerase II subunit I | 105258 | 105261 | 105254 | 29, 15, 11 |
| 19 | POLRMT | RNA polymerase mitochondrial | 099821 | 099822 | 070388 | 38, 29, 27 |
| 19 | RUVBL2 | RuvB like AAA ATPase 2 | 183207 | 104812 | 104826 | 21, 38, 9 |
| 19 | RYR1 | ryanodine receptor 1 | 196218 | 171777 | 104814 | 18, 25, 30 |
| 19 | SAFB | scaffold attachment factor B | 160633 | 130254 | 130255 | 39, 21, 29 |
| 19 | SAFB2 | scaffold attachment factor B2 | 130254 | 223573 | 160633 | 34, 26, 14 |
| 19 | SSC5D | scavenger receptor cysteine rich family member with 5 domains | 179954 | 090971 | 187550 | 1, 6, 36 |
| 19 | SCGB2B2 | secretoglobin family 2B member 2 | 205209 | 142279 | 089335 | 27, 14, 38 |
| 19 | SCAMP4 | secretory carrier membrane protein 4 | 227500 | 129968 | 213638 | 13, 1, 36 |
| 19 | SELENOV | selenoprotein V | 186838 | 090932 | 176401 | 12, 38, |
| 19 | SELENOW | selenoprotein W | 178980 | 105373 | 178928 | |
| 19 | SEMA6B | semaphorin 6B | 167680 | 171236 | 185361 | 30, 37, 35 |
| 19 | SPINT2 | serine peptidase inhibitor, Kunitz type 2 | 167642 | 011332 | 167641 | 27, 28, 37 |
| 19 | SRRM5 | serine/arginine repetitive matrix 5 | 226763 | 124444 | 131116 | 34, 38, 36 |
| 19 | STK11 | serine/threonine kinase 11 | 118046 | 064932 | 099625 | 34, 21, 33 |
| 19 | SERTAD1 | SERTA domain containing 1 | 197019 | 105227 | 167565 | 27, 41, 6 |
| 19 | SERTAD3 | SERTA domain containing 3 | 167565 | 197019 | 090013 | 27, 26, 12 |
| 19 | SARS2 | seryl-tRNA synthetase 2, mitochondrial | 104835 | 262484 | 269547 | 41, 5, 3 |
| 19 | SH2D3A | SH2 domain containing 3A | 125731 | 125733 | 141968 | 9, 4, 24 |
| 19 | SHANK1 | SH3 and multiple ankyrin repeat domains 1 | 161681 | 235034 | 105472 | 2, 23, 8 |
| 19 | SBK2 | SH3 domain binding kinase family member 2 | 187550 | 179954 | 231274 | 12, |
| 19 | SBK3 | SH3 domain binding kinase family member 3 | 231274 | 187550 | 218891 | 38, |
| 19 | SH3GL1 | SH3 domain containing GRB2 like 1, endophilin A2 | 141985 | 008382 | 167670 | 21, 34, 41 |
| 19 | SHKBP1 | SH3KBP1 binding protein 1 | 160410 | 160460 | 090006 | 36, 21, 40 |
| 19 | SHC2 | SHC adaptor protein 2 | 129946 | 183186 | 181781 | 26, 15, 2 |
| 19 | SHISA7 | shisa family member 7 | 187902 | 108106 | 063241 | 8, 17, 12 |
| 19 | SIGLEC10 | sialic acid binding Ig like lectin 10 | 142512 | 262874 | 105366 | 36, 41, 5 |
| 19 | SIGLEC11 | sialic acid binding Ig like lectin 11 | 161640 | 169136 | 105053 | 36, 27, 2 |
| 19 | SIGLEC12 | sialic acid binding Ig like lectin 12 (gene/pseudogene) | 254521 | 213822 | 105492 | 15, 36, 37 |
| 19 | SIGLEC14 | sialic acid binding Ig like lectin 14 | 254415 | 268500 | 182310 | 5, 32, 22 |
| 19 | SIGLEC5 | sialic acid binding Ig like lectin 5 | 105501 | 105497 | 268500 | 5, 36, 4 |
| 19 | SIGLEC6 | sialic acid binding Ig like lectin 6 | 105492 | 254521 | 105497 | 32, 42, 44 |
| 19 | SIGLEC7 | sialic acid binding Ig like lectin 7 | 168995 | 129450 | 105383 | 36, 5, 2 |
| 19 | SIGLEC8 | sialic acid binding Ig like lectin 8 | 105366 | 142512 | 213822 | 30, 36, 39 |
| 19 | SIGLEC9 | sialic acid binding Ig like lectin 9 | 129450 | 142544 | 168995 | 5, 6, 30 |
| 19 | SIGLECL1 | SIGLEC family like 1 | 179213 | 105383 | 142549 | 38, 14, 8 |
| 19 | SIPA1L3 | signal induced proliferation associated 1 like 3 | 105738 | 171804 | 011332 | 22, 29, 26 |
| 19 | SPPL2B | signal peptide peptidase like 2B | 005206 | 130332 | 178297 | 38, 7, 26 |
| 19 | STAP2 | signal transducing adaptor family member 2 | 178078 | 105255 | 008382 | 29, 16, 3 |
| 19 | SIN3B | SIN3 transcription regulator family member B | 127511 | 188039 | 127533 | 26, 22, 32 |
| 19 | SSBP4 | single stranded DNA binding protein 4 | 130511 | 175489 | 105655 | 3, 34, 28 |
| 19 | SIRT2 | sirtuin 2 | 068903 | 187994 | 104825 | 15, 8, 25 |
| 19 | SIRT6 | sirtuin 6 | 077463 | 060566 | 089847 | 27, 3, 36 |
| 19 | SIX5 | SIX homeobox 5 | 177045 | 237452 | 104936 | 12, 26, 2 |
| 19 | SGTA | small glutamine rich tetratricopeptide repeat containing alpha | 104969 | 141873 | 172009 | 21, 26, 34 |
| 19 | SMIM17 | small integral membrane protein 17 | 268182 | 197951 | 127903 | 8, 12, |
| 19 | SMIM24 | small integral membrane protein 24 | 095932 | 141905 | 129932 | |
| 19 | SMIM7 | small integral membrane protein 7 | 214046 | 268790 | 072954 | 34, 30, 13 |
| 19 | SNRPD2 | small nuclear ribonucleoprotein D2 polypeptide | 125743 | 010310 | 011478 | 29, 7, 43 |
| 19 | SNRPA | small nuclear ribonucleoprotein polypeptide A | 077312 | 188493 | 268975 | 21, 3, 26 |
| 19 | SNRNP70 | small nuclear ribonucleoprotein U1 subunit 70 | 104852 | 104848 | 104863 | 3, 44, 39 |
| 19 | SNAPC2 | small nuclear RNA activating complex polypeptide 2 | 104976 | 260001 | 178531 | 9, 33, 34 |
| 19 | SMG9 | SMG9, nonsense mediated mRNA decay factor | 105771 | 124449 | 104783 | 38, 43, 26 |
| 19 | SCN1B | sodium voltage-gated channel beta subunit 1 | 105711 | 089351 | 105707 | 25, 23, 14 |
| 19 | SLC1A5 | solute carrier family 1 member 5 | 105281 | 181027 | 280050 | 27, 3, 16 |
| 19 | SLC1A6 | solute carrier family 1 member 6 | 105143 | 127529 | 160994 | 38, 8, 37 |
| 19 | SLC17A7 | solute carrier family 17 member 7 | 104888 | 261949 | 104872 | 14, 8, 27 |
| 19 | SLC25A23 | solute carrier family 25 member 23 | 125648 | 181240 | 130545 | 40, 9, 14 |
| 19 | SLC25A41 | solute carrier family 25 member 41 | 181240 | 088247 | 125648 | 37, 38, 8 |
| 19 | SLC25A42 | solute carrier family 25 member 42 | 181035 | 105676 | 064545 | 9, 17, 15 |
| 19 | SLC27A1 | solute carrier family 27 member 1 | 130304 | 171773 | 130313 | 26, 11, 27 |
| 19 | SLC27A5 | solute carrier family 27 member 5 | 083807 | 083838 | 119574 | 19, 1, 31 |
| 19 | SLC35E1 | solute carrier family 35 member E1 | 127526 | 085872 | 105085 | 11, 23, 33 |
| 19 | SLC39A3 | solute carrier family 39 member 3 | 141873 | 267001 | 104969 | 21, 9, 6 |
| 19 | SLC44A2 | solute carrier family 44 member 2 | 129353 | 129354 | 129351 | 36, 38, 32 |
| 19 | SLC5A5 | solute carrier family 5 member 5 | 105641 | 105640 | 007080 | 25, 14, 8 |
| 19 | SLC6A16 | solute carrier family 6 member 16 | 063127 | 130529 | 104894 | 38, 26, 24 |
| 19 | SLC7A10 | solute carrier family 7 member 10 | 130876 | 130881 | 245848 | 1, 15, 8 |
| 19 | SLC7A9 | solute carrier family 7 member 9 | 021488 | 173809 | 121289 | 17, 37, 16 |
| 19 | SLC8A2 | solute carrier family 8 member A2 | 118160 | 105419 | 118162 | 8, 28, 14 |
| 19 | SPC24 | SPC24, NDC80 kinetochore complex component | 161888 | 130164 | 197256 | 21, 39, 27 |
| 19 | SPTBN4 | spectrin beta, non-erythrocytic 4 | 160460 | 090013 | 160410 | 26, 28, 2 |
| 19 | SYNE4 | spectrin repeat containing nuclear envelope family member 4 | 181392 | 205138 | 239382 | 31, 23, 39 |
| 19 | SPACA4 | sperm acrosome associated 4 | 177202 | 105523 | 063177 | 38, 8, |
| 19 | SPACA6 | sperm acrosome associated 6 | 182310 | 254415 | 105509 | 28, 2, 38 |
| 19 | SPHK2 | sphingosine kinase 2 | 063176 | 063177 | 105516 | 24, 8, 42 |
| 19 | S1PR2 | sphingosine-1-phosphate receptor 2 | 267534 | 130816 | 105364 | 22, 29, 21 |
| 19 | S1PR4 | sphingosine-1-phosphate receptor 4 | 125910 | 060558 | 125912 | |
| 19 | S1PR5 | sphingosine-1-phosphate receptor 5 | 180739 | 079999 | 130734 | 36, 8, 34 |
| 19 | SPIB | Spi-B transcription factor | 269404 | 142539 | 086967 | 41, 21, 22 |
| 19 | SF3A2 | splicing factor 3a subunit 2 | 104897 | 104886 | 104899 | 21, 26, 9 |
| 19 | SPRED3 | sprouty related EVH1 domain containing 3 | 188766 | 179168 | 130244 | 31, 28, 14 |
| 19 | SUPT5H | SPT5 homolog, DSIF elongation factor subunit | 196235 | 105193 | 105197 | 42, 43, 38 |
| 19 | SHD | Src homology 2 domain containing transforming protein D | 105251 | 105248 | 167664 | 3, 25, 8 |
| 19 | SCAF1 | SR-related CTD associated factor 1 | 126461 | 126458 | 126456 | 21, 14, 12 |
| 19 | SAMD1 | sterile alpha motif domain containing 1 | 141858 | 188032 | 072062 | 12, |
| 19 | SAMD4B | sterile alpha motif domain containing 4B | 179134 | 130755 | 006712 | 28, 26, 18 |
| 19 | SBNO2 | strawberry notch homolog 2 | 064932 | 167468 | 118046 | 34, 27, 1 |
| 19 | STRN4 | striatin 4 | 090372 | 105287 | 181027 | 36, 34, 4 |
| 19 | SDHAF1 | succinate dehydrogenase complex assembly factor 1 | 205138 | 126243 | 181392 | 27, 35, 16 |
| 19 | SULT2A1 | sulfotransferase family 2A member 1 | 105398 | 105392 | 188334 | 2, 19, 29 |
| 19 | SULT2B1 | sulfotransferase family 2B member 1 | 088002 | 142235 | 105523 | 33, 32, 3 |
| 19 | SAE1 | SUMO1 activating enzyme subunit 1 | 142230 | 130749 | 105327 | 10, 38, |
| 19 | SBSN | suprabasin | 189001 | 161249 | 105679 | 18, 35, 24 |
| 19 | SUGP1 | SURP and G-patch domain containing 1 | 105705 | 213996 | 129933 | 39, 38, 36 |
| 19 | SUGP2 | SURP and G-patch domain containing 2 | 064607 | 051128 | 105676 | |
| 19 | SMARCA4 | SWI/SNF related, matrix associated, actin dependent | 127616 | 142444 | 130164 | 43, 18, 21 |
| | | regulator of chromatin, subfamily a, member 4 | | | | |
| 19 | SWSAP1 | SWIM-type zinc finger 7 associated protein 1 | 173928 | 105520 | 187266 | 3, 32, 20 |
| 19 | SYMPK | symplekin | 125755 | 104941 | 170608 | 43, 38, 4 |
| 19 | SYDE1 | synapse defective Rho GTPase homolog 1 | 105137 | 094661 | 105135 | 34, 32, 6 |
| 19 | SYNGR4 | synaptogyrin 4 | 105467 | 161558 | 105438 | 3, 38, 10 |
| 19 | SYCE2 | synaptonemal complex central element protein 2 | 161860 | 105607 | 179115 | 21, 38, 32 |
| 19 | SYT3 | synaptotagmin 3 | 213023 | 131409 | 235034 | 2, 22, 14 |
| 19 | SYT5 | synaptotagmin 5 | 129990 | 167646 | 080031 | |
| 19 | SYCN | syncoilin | 179751 | 188505 | 197110 | 28, 38, |
| 19 | STX10 | syntaxin 10 | 104915 | 160877 | 160888 | 1, 43, 20 |
| 19 | STXBP2 | syntaxin binding protein 2 | 076944 | 174788 | 104918 | 5, 39, 9 |
| 19 | TARM1 | T cell-interacting, activating receptor on myeloid cells 1 | 248385 | 189068 | 170909 | 10, 38, |
| 19 | TBC1D17 | TBC1 domain family member 17 | 104946 | 204673 | 104951 | 2, 39, 36 |
| 19 | TFPT | TCF3 fusion partner | 105619 | 170906 | 105618 | 1, 13, 29 |
| 19 | TEAD2 | TEA domain transcription factor 2 | 074219 | 104894 | 104901 | 34, 1, 29 |
| 19 | TSHZ3 | teashirt zinc finger homeobox 3 | 121297 | 198597 | 168813 | 45, 29, 1 |
| 19 | TEX101 | testis expressed 101 | 131126 | 204936 | 124466 | 38, 14, 8 |
| 19 | TSSK6 | testis specific serine kinase 6 | 178093 | 167491 | 186010 | 38, 4, 27 |
| 19 | TSKS | testis specific serine kinase substrate | 126467 | 169169 | 196961 | 38, 32, 28 |
| 19 | TPRX1 | tetrapeptide repeat homeobox 1 | 178928 | 178980 | 105392 | 14, 38, |
| 19 | TSPAN16 | tetraspanin 16 | 130167 | 130173 | 105514 | 38, 5, 8 |
| 19 | TTC9B | tetratricopeptide repeat domain 9B | 174521 | 130758 | 105219 | 6, 8, 14 |
| 19 | THAP8 | THAP domain containing 8 | 161277 | 105270 | 075702 | 25, 38, 35 |
| 19 | THEG | theg spermatid protein | 105549 | 105556 | 183186 | 38, 23, 20 |
| 19 | THOP1 | thimet oligopeptidase 1 | 172009 | 104969 | 172006 | 41, 27, 3 |
| 19 | TBXA2R | thromboxane A2 receptor | 006638 | 179855 | 105298 | 17, 44, 39 |
| 19 | TRIP10 | thyroid hormone receptor interactor 10 | 125733 | 125734 | 125731 | 25, 18, 6 |
| 19 | TJP3 | tight junction protein 3 | 105289 | 186111 | 011132 | 42, 27, 24 |
| 19 | TINCR | Tissue differentiation-inducing non-protein coding RNA | 223573 | 105428 | 130254 | 32, 15, 33 |
| 19 | TNFAIP8L1 | TNF alpha induced protein 8 like 1 | 185361 | 167680 | 074842 | 27, 15, 36 |
| 19 | TICAM1 | toll like receptor adaptor molecule 1 | 127666 | 141965 | 105355 | 21, 6, 22 |
| 19 | TRAPPC2B | Trafficking protein particle complex 2B | 256060 | 152433 | 268133 | |
| 19 | TRAPPC5 | trafficking protein particle complex 5 | 181029 | 269711 | 104921 | 34, 20, 22 |
| 19 | TRAPPC6A | trafficking protein particle complex 6A | 007255 | 179846 | 189114 | 22, 1, 6 |
| 19 | TECR | trans-2,3-enoyl-CoA reductase | 099797 | 132002 | 099795 | 1, 9, 13 |
| 19 | TCF3 | transcription factor 3 | 071564 | 127540 | 205922 | 22, 21, 41 |
| 19 | TLE2 | transducin like enhancer of split 2 | 065717 | 104953 | 104964 | 33, 23, 14 |
| 19 | TLE6 | transducin like enhancer of split 6 | 104953 | 175691 | 065717 | 3, 21, 2 |
| 19 | TGFB1 | transforming growth factor beta 1 | 105329 | 105323 | 142039 | 34, 5, 6 |
| 19 | TGFBR3L | transforming growth factor beta receptor 3 like | 260001 | 076984 | 104976 | 44, 28, 14 |
| 19 | TRPM4 | transient receptor potential cation channel subfamily | 130529 | 130528 | 063127 | 26, 41, 33 |
| | | M member 4 | | | | |
| 19 | TIMM13 | translocase of inner mitochondrial membrane 13 | 099800 | 178297 | 176619 | 2, 25, 33 |
| 19 | TIMM29 | translocase of inner mitochondrial membrane 29 | 142444 | 130733 | 127616 | |
| 19 | TIMM44 | translocase of inner mitochondrial membrane 44 | 104980 | 178531 | 066044 | 3, 13, 43 |
| 19 | TIMM50 | translocase of inner mitochondrial membrane 50 | 105197 | 196235 | 090932 | 38, 12, |
| 19 | TOMM40 | translocase of outer mitochondrial membrane 40 | 130204 | 130202 | 130203 | 41, 37, 34 |
| 19 | TM6SF2 | transmembrane 6 superfamily member 2 | 213996 | 187664 | 105705 | 16, 27, 19 |
| 19 | TMIGD2 | transmembrane and immunoglobulin domain containing 2 | 167664 | 105251 | 105255 | 6, 39, 32 |
| 19 | TMC4 | transmembrane channel like 4 | 167608 | 105617 | 125505 | 33, 27, 23 |
| 19 | TMED1 | transmembrane p24 trafficking protein 1 | 099203 | 079805 | 214212 | 9, 35, 31 |
| 19 | TMPRSS9 | transmembrane protease, serine 9 | 178297 | 005206 | 099800 | |
| 19 | TMEM143 | transmembrane protein 143 | 161558 | 142227 | 105467 | 15, 39, 13 |
| 19 | TMEM145 | transmembrane protein 145 | 167619 | 188368 | 105429 | 14, 12, 8 |
| 19 | TMEM147 | transmembrane protein 147 | 105677 | 105679 | 105675 | 1, 3, 28 |
| 19 | TMEM150B | transmembrane protein 150B | 180061 | 160469 | 133247 | 5, 17, 16 |
| 19 | TMEM160 | transmembrane protein 160 | 130748 | 130751 | 130749 | 1, 28, 26 |
| 19 | TMEM161A | transmembrane protein 161A | 064545 | 181035 | 064489 | 9, 13, 21 |
| 19 | TMEM190 | transmembrane protein 190 | 160472 | 095752 | 233493 | 30, 38, 27 |
| 19 | TMEM205 | transmembrane protein 205 | 105518 | 105514 | 183401 | 29, 27, 41 |
| 19 | TMEM221 | transmembrane protein 221 | 188051 | 141971 | 269035 | 37, 23, 17 |
| 19 | TMEM238 | transmembrane protein 238 | 233493 | 160472 | 108107 | 17, 16, 23 |
| 19 | TMEM259 | transmembrane protein 259 | 182087 | 116032 | 064666 | 29, 22, 21 |
| 19 | TMEM38A | transmembrane protein 38A | 072954 | 214046 | 188039 | 18, 25, 21 |
| 19 | TMEM59L | transmembrane protein 59 like | 105696 | 006015 | 167487 | 8, 34, 31 |
| 19 | TMEM86B | transmembrane protein 86B | 180089 | 080031 | 105063 | 41, 6, 39 |
| 19 | TMEM91 | transmembrane protein 91 | 142046 | 255730 | 123810 | 40, 14, 17 |
| 19 | TNPO2 | transportin 2 | 105576 | 132004 | 123144 | 7, 41, 43 |
| 19 | TRIM28 | tripartite motif containing 28 | 130726 | 119574 | 130724 | 27, 35, 21 |
| 19 | TRMT1 | tRNA methyltransferase 1 | 104907 | 104903 | 160877 | 36, 2, 39 |
| 19 | TSEN34 | tRNA splicing endonuclease subunit 34 | 170892 | 125505 | 170889 | 27, 43, 6 |
| 19 | TPM4 | tropomyosin 4 | 167460 | 176231 | 167461 | 6, 23, 19 |
| 19 | TNNI3 | troponin I3, cardiac type | 129991 | 105048 | 267110 | 15, 20, 19 |
| 19 | TNNT1 | troponin T1, slow skeletal type | 105048 | 125503 | 129991 | 25, 24, 15 |
| 19 | TULP2 | tubby like protein 2 | 104804 | 087074 | 104805 | 38, 33, 19 |
| 19 | TUBB4A | tubulin beta 4A class IVa | 104833 | 205744 | 125657 | 8, 2, 17 |
| 19 | TBCB | tubulin folding cofactor B | 105254 | 105258 | 126247 | 28, 41, 35 |
| 19 | TPGS1 | tubulin polyglutamylase complex subunit 1 | 141933 | 099866 | 099804 | 22, 28, 37 |
| 19 | TDRD12 | tudor domain containing 12 | 173809 | 213965 | 021488 | |
| 19 | TNFSF14 | tumor necrosis factor superfamily member 14 | 125735 | 125726 | 125730 | 19, 36, 23 |
| 19 | TNFSF9 | tumor necrosis factor superfamily member 9 | 125657 | 104833 | 125726 | 9, 26, 37 |
| 19 | TTYH1 | tweety family member 1 | 167614 | 167613 | 167615 | 8, 26, 38 |
| 19 | TYROBP | TYRO protein tyrosine kinase binding protein | 011600 | 126264 | 126243 | 24, 1, 11 |
| 19 | TYK2 | tyrosine kinase 2 | 105397 | 076662 | 105401 | 43, 42, 39 |
| 19 | U2AF1L4 | U2 small nuclear RNA auxiliary factor 1 like 4 | 161265 | 267120 | 205155 | 29, 36, 22 |
| 19 | U2AF2 | U2 small nuclear RNA auxiliary factor 2 | 063244 | 173581 | 063245 | 34, 22, 14 |
| 19 | UQCR11 | ubiquinol-cytochrome c reductase, complex III subunit XI | 127540 | 267059 | 071564 | 25, 15, 43 |
| 19 | UQCRFS1 | ubiquinol-cytochrome c reductase, Rieske iron-sulfur | 169021 | 213096 | 187135 | 2, 24, 27 |
| | | polypeptide 1 | | | | |
| 19 | UBA52 | ubiquitin A-52 residue ribosomal protein fusion product 1 | 221983 | 105700 | 006016 | 12, 25, 43 |
| 19 | UBE2M | ubiquitin conjugating enzyme E2 M | 130725 | 130724 | 099326 | 37, 27, 9 |
| 19 | UBE2S | ubiquitin conjugating enzyme E2 S | 108106 | 108107 | 187902 | 41, 21, 35 |
| 19 | UBL5 | ubiquitin like 5 | 198258 | 127452 | 127445 | 43, 15, 31 |
| 19 | UBA2 | ubiquitin like modifier activating enzyme 2 | 126261 | 126249 | 142279 | 45, 3, 37 |
| 19 | UHRF1 | ubiquitin like with PHD and ring finger domains 1 | 276043 | 205784 | 127663 | 39, 22, 12 |
| 19 | USP29 | ubiquitin specific peptidase 29 | 131864 | 198300 | 141946 | 38, |
| 19 | UBXN6 | UBX domain protein 6 | 167671 | 167670 | 167674 | 34, 29, 26 |
| 19 | B3GNT3 | UDP-GlcNAc: betaGal beta-1,3-N-acetylglucosaminyltransferase 3 | 179913 | 130475 | 248099 | 16, |
| 19 | B3GNT8 | UDP-GlcNAc: betaGal beta-1,3-N-acetylglucosaminyltransferase 8 | 177191 | 248098 | 105341 | |
| 19 | UNC13A | unc-13 homolog A | 130477 | 130309 | 130479 | 26, 31, 8 |
| 19 | USE1 | unconventional SNARE in the ER 1 | 053501 | 099331 | 099330 | 41, 37, 3 |
| 19 | UPF1 | UPF1, RNA helicase and ATPase | 005007 | 105664 | 223802 | 22, 34, 38 |
| 19 | USF2 | upstream transcription factor 2, c-fos interacting | 105698 | 105699 | 105697 | 34, 18, 37 |
| 19 | URI1 | URI1, prefoldin like chaperone | 105176 | 105173 | 198597 | |
| 19 | UPK1A | uroplakin 1A | 105668 | 126267 | 011590 | 4, 33, 44 |
| 19 | USHBP1 | USH1 protein network component harmonin binding protein 1 | 130307 | 269095 | 105393 | 1, 36, 15 |
| 19 | VRK3 | vaccinia related kinase 3 | 105053 | 161640 | 142528 | 38, 21, 39 |
| 19 | VASP | vasodilator-stimulated phosphoprotein | 125753 | 213889 | 125741 | 41, 5, 7 |
| 19 | VAV1 | vav guanine nucleotide exchange factor 1 | 141968 | 125731 | 174837 | 39, 36, 5 |
| 19 | VMAC | vimentin-type intermediate filament associated coiled-coil | 187650 | 267314 | 105519 | 34, 9, 39 |
| | | protein | | | | |
| 19 | VN1R1 | vomeronasal 1 receptor 1 | 178201 | 268163 | 197128 | 36, 33, 8 |
| 19 | VN1R2 | vomeronasal 1 receptor 2 | 196131 | 197928 | 228567 | |
| 19 | VN1R4 | vomeronasal 1 receptor 4 | 228567 | 196131 | 163098 | |
| 19 | VSIG10L | V-set and immunoglobulin domain containing 10 like | 186806 | 142549 | 105379 | 26, 31, 6 |
| 19 | VSTM1 | V-set and transmembrane domain containing 1 | 189068 | 130433 | 248385 | 5, 10, 8 |
| 19 | VSTM2B | V-set and transmembrane domain containing 2B | 187135 | 169021 | 105171 | 8, 32, |
| 19 | WDR18 | WD repeat domain 18 | 065268 | 116017 | 116032 | 9, 27, 5 |
| 19 | WDR62 | WD repeat domain 62 | 075702 | 161277 | 105261 | 38, 22, 21 |
| 19 | WDR83 | WD repeat domain 83 | 123154 | 269590 | 105583 | 1, 43, 6 |
| 19 | WDR83OS | WD repeat domain 83 opposite strand | 105583 | 123154 | 095059 | 29, 31, 30 |
| 19 | WDR87 | WD repeat domain 87 | 171804 | 189144 | 105738 | 38, |
| 19 | WDR88 | WD repeat domain 88 | 166359 | 076650 | 130881 | 38, 14, 8 |
| 19 | WIZ | widely interspaced zinc finger motifs | 011451 | 011243 | 105122 | 34, 35, 14 |
| 19 | WTIP | Wilms tumor 1 interacting protein | 142279 | 126261 | 205209 | 27, 26, 45 |
| 19 | XAB2 | XPA binding protein 2 | 076924 | 076826 | 229833 | 40, 5, 21 |
| 19 | XRCC1 | X-ray repair cross complementing 1 | 073050 | 176472 | 268361 | 21, 14, 9 |
| 19 | YIPF2 | Yip1 domain family member 2 | 130733 | 142453 | 142444 | 26, 33, 9 |
| 19 | YIF1B | Yip1 interacting factor homolog B, membrane trafficking | 167645 | 167644 | 099337 | 26, 27, 6 |
| | | protein | | | | |
| 19 | YJEFN3 | YjeF N-terminal domain containing 3 | 250067 | 258674 | 160161 | 1, 4, 38 |
| 19 | ZFP14 | ZFP14 zinc finger protein | 142065 | 167635 | 181007 | 1, 11, 30 |
| 19 | ZFP28 | ZFP28 zinc finger protein | 196867 | 196263 | 197016 | 25, 6, 40 |
| 19 | ZFP30 | ZFP30 zinc finger protein | 120784 | 180479 | 196381 | 1, 30, 22 |
| 19 | ZFP36 | ZFP36 ring finger protein | 128016 | 063322 | 090924 | 26, 28, 1 |
| 19 | ZFP82 | ZFP82 zinc finger protein | 181007 | 142065 | 186017 | 30, 9, 45 |
| 19 | ZNRF4 | zinc and ring finger 4 | 105428 | 283229 | 223573 | 38, 8, |
| 19 | ZBTB32 | zinc finger and BTB domain containing 32 | 011590 | 105668 | 272333 | |
| 19 | ZBTB45 | zinc finger and BTB domain containing 45 | 119574 | 083807 | 130726 | 37, 36, 3 |
| 19 | ZBTB7A | zinc finger and BTB domain containing 7A | 178951 | 105229 | 126934 | 22, 33, 14 |
| 19 | ZSCAN1 | zinc finger and SCAN domain containing 1 | 152467 | 166704 | 176293 | 14, 38, 8 |
| 19 | ZSCAN18 | zinc finger and SCAN domain containing 18 | 121413 | 176293 | 181894 | 8, 36, 2 |
| 19 | ZSCAN22 | zinc finger and SCAN domain containing 22 | 182318 | 142396 | 121410 | 11, 34, 3 |
| 19 | ZSCAN4 | zinc finger and SCAN domain containing 4 | 180532 | 121417 | 204519 | 32, 44, |
| 19 | ZSCAN5A | zinc finger and SCAN domain containing 5A | 131848 | 204532 | 267710 | 38, 42, 13 |
| 19 | ZSCAN5B | zinc finger and SCAN domain containing 5B | 197213 | 197487 | 204532 | 38, |
| 19 | ZSCAN5C | Zinc finger and SCAN domain containing 5C | 204532 | 197213 | 131848 | |
| 19 | ZC3H4 | zinc finger CCCH-type containing 4 | 130749 | 130748 | 142230 | 26, 34, 29 |
| 19 | ZNF788 | zinc finger family member 788 | 214189 | 223547 | 132010 | 39, 17, 38 |
| 19 | ZIM2 | zinc finger imprinted 2 | 269699 | 127903 | 198300 | |
| 19 | ZIM3 | zinc finger imprinted 3 | 141946 | 131864 | 258873 | |
| 19 | ZNF100 | zinc finger protein 100 | 197020 | 197013 | 198521 | 22, 37, 2 |
| 19 | ZNF101 | zinc finger protein 101 | 181896 | 105726 | 105708 | 36, 39, 3 |
| 19 | ZNF112 | zinc finger protein 112 | 062370 | 159915 | 267173 | 29, 42, 40 |
| 19 | ZNF114 | zinc finger protein 114 | 178150 | 185453 | 105483 | 40, 35, 19 |
| 19 | ZNF121 | zinc finger protein 121 | 197961 | 130818 | 171469 | 31, 3, 10 |
| 19 | ZNF132 | zinc finger protein 132 | 131849 | 171574 | 249471 | 38, 3, 21 |
| 19 | ZNF134 | zinc finger protein 134 | 213762 | 183647 | 121417 | 1, 45, 42 |
| 19 | ZNF135 | zinc finger protein 135 | 176293 | 152467 | 121413 | 31, 26, 25 |
| 19 | ZNF136 | zinc finger protein 136 | 196646 | 257591 | 197857 | 18, 42, 36 |
| 19 | ZNF14 | zinc finger protein 14 | 105708 | 181896 | 081665 | 2, 39, 3 |
| 19 | ZNF146 | zinc finger protein 146 | 167635 | 196357 | 142065 | 42, 22, 4 |
| 19 | ZNF154 | zinc finger protein 154 | 179909 | 269026 | 083814 | 40, 42, 39 |
| 19 | ZNF155 | zinc finger protein 155 | 204920 | 159905 | 159882 | 36, 4, 24 |
| 19 | ZNF160 | zinc finger protein 160 | 170949 | 268964 | 170954 | |
| 19 | ZNF17 | zinc finger protein 17 | 186272 | 268533 | 186230 | 42, 2, 36 |
| 19 | ZNF175 | zinc finger protein 175 | 105497 | 105492 | 105501 | 26, 31, 34 |
| 19 | ZNF177 | zinc finger protein 177 | 188629 | 270011 | 174652 | 8, 39, 38 |
| 19 | ZNF180 | zinc finger protein 180 | 167384 | 278318 | 273777 | 29, 36, 4 |
| 19 | ZNF181 | zinc finger protein 181 | 197841 | 089335 | 153896 | 39, 15, 37 |
| 19 | ZNF20 | zinc finger protein 20 | 132010 | 214189 | 213297 | 38, 41, 39 |
| 19 | ZNF208 | zinc finger protein 208 | 160321 | 198521 | 197134 | 29, 42, 40 |
| 19 | ZNF211 | zinc finger protein 211 | 121417 | 213762 | 180532 | 11, 5, 36 |
| 19 | ZNF221 | zinc finger protein 221 | 159905 | 124459 | 204920 | 45, 37, 14 |
| 19 | ZNF222 | zinc finger protein 222 | 159885 | 159882 | 267022 | 15, 32, 17 |
| 19 | ZNF223 | zinc finger protein 223 | 178386 | 267022 | 186026 | 2, 3, 17 |
| 19 | ZNF224 | zinc finger protein 224 | 267680 | 186026 | 256294 | |
| 19 | ZNF225 | zinc finger protein 225 | 256294 | 267680 | 263002 | 26, 39, 22 |
| 19 | ZNF226 | zinc finger protein 226 | 167380 | 263002 | 131115 | 31, 13, 39 |
| 19 | ZNF227 | zinc finger protein 227 | 131115 | 167380 | 159917 | |
| 19 | ZNF229 | zinc finger protein 229 | 278318 | 267508 | 167384 | 14, 42, 17 |
| 19 | ZNF230 | zinc finger protein 230 | 159882 | 204920 | 159885 | 40, 22, 4 |
| 19 | ZNF233 | zinc finger protein 233 | 159915 | 159917 | 062370 | 26, 11, 24 |
| 19 | ZNF234 | zinc finger protein 234 | 263002 | 256294 | 167380 | 28, 36, 2 |
| 19 | ZNF235 | zinc finger protein 235 | 159917 | 131115 | 159915 | 31, 42, 39 |
| 19 | ZNF253 | zinc finger protein 253 | 256771 | 081665 | 184635 | |
| 19 | ZNF254 | zinc finger protein 254 | 213096 | 213967 | 169021 | 4, 34, 28 |
| 19 | ZNF256 | zinc finger protein 256 | 152454 | 196724 | 177025 | 1, 45, 4 |
| 19 | ZNF257 | zinc finger protein 257 | 197134 | 160321 | 196109 | 39, 17, 45 |
| 19 | ZNF260 | zinc finger protein 260 | 254004 | 186017 | 186020 | 11, 4, 12 |
| 19 | ZNF264 | zinc finger protein 264 | 083844 | 258873 | 105146 | 36, 43, 2 |
| 19 | ZNF266 | zinc finger protein 266 | 174652 | 188629 | 198028 | 40, 16, 22 |
| 19 | ZNF274 | zinc finger protein 274 | 171606 | 181894 | 198131 | 39, 29, 42 |
| 19 | ZNF28 | zinc finger protein 28 | 198538 | 189190 | 204604 | |
| 19 | ZNF283 | zinc finger protein 283 | 167637 | 159871 | 176222 | 13, 42, 40 |
| 19 | ZNF284 | zinc finger protein 284 | 186026 | 178386 | 267680 | 36, 6, 8 |
| 19 | ZNF285 | zinc finger protein 285 | 267508 | 267173 | 278318 | 36, 15, 40 |
| 19 | ZNF296 | zinc finger protein 296 | 170684 | 104859 | 142252 | 21, 3, 28 |
| 19 | ZNF30 | zinc finger protein 30 | 168661 | 153896 | 180884 | 22, 23, 7 |
| 19 | ZNF302 | zinc finger protein 302 | 089335 | 205209 | 197841 | 11, 2, 22 |
| 19 | ZNF304 | zinc finger protein 304 | 131845 | 178229 | 152433 | 45, 23, 37 |
| 19 | ZNF317 | zinc finger protein 317 | 130803 | 170920 | 188000 | 43, 24, 42 |
| 19 | ZNF320 | zinc finger protein 320 | 182986 | 204604 | 283088 | 42, 29, 18 |
| 19 | ZNF324 | zinc finger protein 324 | 083812 | 249471 | 083838 | 39, 24, 8 |
| 19 | ZNF324B | zinc finger protein 324B | 249471 | 131849 | 083812 | 2, 27, 38 |
| 19 | ZNF329 | zinc finger protein 329 | 181894 | 121413 | 171606 | 11, 23, 31 |
| 19 | ZNF331 | zinc finger protein 331 | 130844 | 198346 | 204595 | 24, 12, 39 |
| 19 | ZNF333 | zinc finger protein 333 | 160961 | 131355 | 127507 | 1, 27, 8 |
| 19 | ZNF345 | zinc finger protein 345 | 251247 | 197863 | 185869 | 39, 38, 24 |
| 19 | ZNF347 | zinc finger protein 347 | 197937 | 170954 | 197497 | 42, 25, 5 |
| 19 | ZNF350 | zinc finger protein 350 | 256683 | 176024 | 197619 | 2, 13, 25 |
| 19 | ZNF358 | zinc finger protein 358 | 198816 | 267952 | 090674 | 27, 28, 25 |
| 19 | ZNF382 | zinc finger protein 382 | 161298 | 186020 | 197808 | 30, 23, 38 |
| 19 | ZNF383 | zinc finger protein 383 | 188283 | 245680 | 181666 | |
| 19 | ZNF404 | zinc finger protein 404 | 176222 | 167637 | 124459 | 13, 14, 28 |
| 19 | ZNF414 | zinc finger protein 414 | 133250 | 133246 | 142347 | 29, 22, 34 |
| 19 | ZNF415 | zinc finger protein 415 | 170954 | 170949 | 197937 | 29, 38, 8 |
| 19 | ZNF416 | zinc finger protein 416 | 083817 | 251369 | 171649 | 1, 22, 9 |
| 19 | ZNF417 | zinc finger protein 417 | 173480 | 269476 | 196724 | 18, 6, 40 |
| 19 | ZNF418 | zinc finger protein 418 | 196724 | 173480 | 152454 | 39, 38, 22 |
| 19 | ZNF419 | zinc finger protein 419 | 105136 | 197128 | 268107 | 45, 39, 41 |
| 19 | ZNF420 | zinc finger protein 420 | 197050 | 198453 | 267360 | 1, 26, 39 |
| 19 | ZNF426 | zinc finger protein 426 | 130818 | 198028 | 197961 | 4, 13, 3 |
| 19 | ZNF428 | zinc finger protein 428 | 131116 | 226763 | 105767 | 26, 44, 12 |
| 19 | ZNF429 | zinc finger protein 429 | 197013 | 269237 | 197020 | |
| 19 | ZNF43 | zinc finger protein 43 | 198521 | 197020 | 160321 | 39, 38, 23 |
| 19 | ZNF430 | zinc finger protein 430 | 118620 | 105750 | 160352 | 36, 4, 31 |
| 19 | ZNF431 | zinc finger protein 431 | 196705 | 160352 | 182141 | 1, 31, 39 |
| 19 | ZNF432 | zinc finger protein 432 | 256087 | 142556 | 197608 | 5, 42, 28 |
| 19 | ZNF433 | zinc finger protein 433 | 197647 | 197054 | 257355 | 38, 12, 19 |
| 19 | ZNF439 | zinc finger protein 439 | 171291 | 171295 | 198429 | |
| 19 | ZNF44 | zinc finger protein 44 | 197857 | 196646 | 188868 | 1, 30, 41 |
| 19 | ZNF440 | zinc finger protein 440 | 171295 | 177599 | 171291 | 24, 42, 2 |
| 19 | ZNF441 | zinc finger protein 441 | 197044 | 197933 | 177599 | 4, 40, 23 |
| 19 | ZNF442 | zinc finger protein 442 | 198342 | 188868 | 268744 | 42, 2, 36 |
| 19 | ZNF443 | zinc finger protein 443 | 180855 | 268870 | 269755 | 36, 4, 39 |
| 19 | ZNF444 | zinc finger protein 444 | 167685 | 142409 | 197487 | 34, 39, 36 |
| 19 | ZNF446 | zinc finger protein 446 | 083838 | 083812 | 083807 | 43, 4, 26 |
| 19 | ZNF45 | zinc finger protein 45 | 124459 | 176222 | 159905 | 29, 42, 24 |
| 19 | ZNF460 | zinc finger protein 460 | 197714 | 204524 | 178229 | 40, 42, 1 |
| 19 | ZNF461 | zinc finger protein 461 | 197808 | 161298 | 189042 | 4, 22, 42 |
| 19 | ZNF468 | zinc finger protein 468 | 204604 | 198538 | 182986 | 42, 32, 3 |
| 19 | ZNF470 | zinc finger protein 470 | 197016 | 196867 | 197951 | 26, 14, 2 |
| 19 | ZNF471 | zinc finger protein 471 | 196263 | 198046 | 196867 | 2, 33, 15 |
| 19 | ZNF473 | zinc finger protein 473 | 142528 | 105053 | 161652 | 38, 26, 3 |
| 19 | ZNF480 | zinc finger protein 480 | 198464 | 196214 | 167554 | 24, 28, 39 |
| 19 | ZNF486 | zinc finger protein 486 | 256229 | 213988 | 237440 | 40, 27, 32 |
| 19 | ZNF490 | zinc finger protein 490 | 188033 | 269693 | 173875 | 29, 21, 7 |
| 19 | ZNF491 | zinc finger protein 491 | 177599 | 197044 | 171295 | 38, 14, 8 |
| 19 | ZNF492 | zinc finger protein 492 | 229676 | 197360 | 213973 | 25, 17, 38 |
| 19 | ZNF493 | zinc finger protein 493 | 196268 | 172687 | 269237 | 42, 2, 40 |
| 19 | ZNF497 | zinc finger protein 497 | 174586 | 121410 | 152475 | 27, 14, 22 |
| 19 | ZNF506 | zinc finger protein 506 | 081665 | 105708 | 256771 | 3, 39, 42 |
| 19 | ZNF507 | zinc finger protein 507 | 168813 | 121297 | 178904 | 11, 22, 31 |
| 19 | ZNF524 | zinc finger protein 524 | 171443 | 179943 | 261221 | 37, 28, 10 |
| 19 | ZNF525 | zinc finger protein 525 | 203326 | 163098 | 213799 | 4, 22, 30 |
| 19 | ZNF526 | zinc finger protein 526 | 167625 | 160570 | 105723 | 41, 39, 6 |
| 19 | ZNF527 | zinc finger protein 527 | 189164 | 181666 | 196437 | 36, 37, 45 |
| 19 | ZNF528 | zinc finger protein 528 | 167555 | 221923 | 198633 | 42, 7, 36 |
| 19 | ZNF529 | zinc finger protein 529 | 186020 | 254004 | 161298 | |
| 19 | ZNF530 | zinc finger protein 530 | 183647 | 171649 | 213762 | 7, 33, 37 |
| 19 | ZNF534 | zinc finger protein 534 | 198633 | 167555 | 258405 | |
| 19 | ZNF536 | zinc finger protein 536 | 198597 | 105176 | 121297 | 8, 30, 35 |
| 19 | ZNF540 | zinc finger protein 540 | 171817 | 188227 | 180479 | 8, 29, 39 |
| 19 | ZNF541 | zinc finger protein 541 | 118156 | 105402 | 063169 | 2, 35, 17 |
| 19 | ZNF543 | zinc finger protein 543 | 178229 | 197714 | 131845 | 40, 30, 24 |
| 19 | ZNF544 | zinc finger protein 544 | 198131 | 171606 | 283515 | 24, 38, 13 |
| 19 | ZNF546 | zinc finger protein 546 | 187187 | 013275 | 128000 | 31, 36, 38 |
| 19 | ZNF547 | zinc finger protein 547 | 152433 | 131845 | 256060 | 1, 30, 43 |
| 19 | ZNF548 | zinc finger protein 548 | 188785 | 268133 | 269533 | 38, 13, 39 |
| 19 | ZNF549 | zinc finger protein 549 | 121406 | 152439 | 251369 | 40, 45, 42 |
| 19 | ZNF550 | zinc finger protein 550 | 251369 | 121406 | 083817 | 1, 31, 6 |
| 19 | ZNF551 | zinc finger protein 551 | 204519 | 180532 | 269026 | 1, 25, 42 |
| 19 | ZNF552 | zinc finger protein 552 | 178935 | 083828 | 268750 | 40, 33, 36 |
| 19 | ZNF554 | zinc finger protein 554 | 172006 | 172009 | 186300 | 26, 33, 36 |
| 19 | ZNF555 | zinc finger protein 555 | 186300 | 172006 | 172000 | 3, 23, 38 |
| 19 | ZNF556 | zinc finger protein 556 | 172000 | 186300 | 171970 | 25, 19, 12 |
| 19 | ZNF557 | zinc finger protein 557 | 130544 | 182315 | 171105 | 29, 40, 45 |
| 19 | ZNF558 | zinc finger protein 558 | 167785 | 181733 | 170948 | 13, 31, 37 |
| 19 | ZNF559 | zinc finger protein 559 | 188321 | 196110 | 270011 | |
| 19 | ZNF560 | zinc finger protein 560 | 198028 | 174652 | 130818 | 6, 38, 35 |
| 19 | ZNF561 | zinc finger protein 561 | 171469 | 197961 | 279471 | 42, 45, 44 |
| 19 | ZNF562 | zinc finger protein 562 | 171466 | 279471 | 224689 | 40, 45, 13 |
| 19 | ZNF563 | zinc finger protein 563 | 188868 | 197857 | 198342 | 24, 38, 7 |
| 19 | ZNF564 | zinc finger protein 564 | 249709 | 196826 | 269693 | 31, 45, 24 |
| 19 | ZNF565 | zinc finger protein 565 | 196357 | 161281 | 167635 | |
| 19 | ZNF566 | zinc finger protein 566 | 186017 | 181007 | 254004 | 13, 4, 45 |
| 19 | ZNF567 | zinc finger protein 567 | 189042 | 197808 | 267041 | 11, 27, 39 |
| 19 | ZNF568 | zinc finger protein 568 | 198453 | 185869 | 197050 | |
| 19 | ZNF569 | zinc finger protein 569 | 196437 | 189164 | 171827 | 31, 24, 42 |
| 19 | ZNF57 | zinc finger protein 57 | 171970 | 172000 | 175691 | 9, 3, 21 |
| 19 | ZNF570 | zinc finger protein 570 | 171827 | 196437 | 188227 | 38, |
| 19 | ZNF571 | zinc finger protein 571 | 180479 | 171817 | 120784 | 26, 25, 38 |
| 19 | ZNF573 | zinc finger protein 573 | 189144 | 198182 | 171804 | 11, 25, 39 |
| 19 | ZNF574 | zinc finger protein 574 | 105732 | 105737 | 028277 | 21, 38, 41 |
| 19 | ZNF575 | zinc finger protein 575 | 176472 | 105755 | 073050 | 36, 40, 16 |
| 19 | ZNF576 | zinc finger protein 576 | 124444 | 167378 | 226763 | 43, 26, 2 |
| 19 | ZNF577 | zinc finger protein 577 | 161551 | 187474 | 283617 | 11, 33, 38 |
| 19 | ZNF578 | zinc finger protein 578 | 258405 | 198633 | 198482 | 31, 45, 38 |
| 19 | ZNF579 | zinc finger protein 579 | 218891 | 231274 | 179943 | 12, 21, 44 |
| 19 | ZNF580 | zinc finger protein 580 | 213015 | 179922 | 171425 | 34, 28, 3 |
| 19 | ZNF581 | zinc finger protein 581 | 171425 | 213015 | 173581 | 1, 43, 41 |
| 19 | ZNF582 | zinc finger protein 582 | 018869 | 267710 | 198440 | 36, 17, 38 |
| 19 | ZNF583 | zinc finger protein 583 | 198440 | 018869 | 198046 | |
| 19 | ZNF584 | zinc finger protein 584 | 171574 | 269855 | 131849 | 3, 2, 36 |
| 19 | ZNF585A | zinc finger protein 585A | 196967 | 267360 | 245680 | |
| 19 | ZNF585B | zinc finger protein 585B | 245680 | 196967 | 188283 | 2, 7, 45 |
| 19 | ZNF586 | zinc finger protein 586 | 083828 | 152443 | 178935 | 39, 26, 11 |
| 19 | ZNF587 | zinc finger protein 587 | 198466 | 204514 | 269476 | 1, 4, 23 |
| 19 | ZNF587B | zinc finger protein 587B | 269343 | 268750 | 204514 | 29, 13, 23 |
| 19 | ZNF599 | zinc finger protein 599 | 153896 | 197841 | 168661 | 26, 31, 17 |
| 19 | ZNF600 | zinc finger protein 600 | 189190 | 213020 | 198538 | 30, 21, 37 |
| 19 | ZNF606 | zinc finger protein 606 | 166704 | 177025 | 152467 | 39, 4, 44 |
| 19 | ZNF607 | zinc finger protein 607 | 198182 | 267552 | 189144 | 37, 42, 38 |
| 19 | ZNF610 | zinc finger protein 610 | 167554 | 198464 | 221923 | 2, 14, 38 |
| 19 | ZNF611 | zinc finger protein 611 | 213020 | 167766 | 189190 | 33, 42, 36 |
| 19 | ZNF613 | zinc finger protein 613 | 176024 | 198093 | 256683 | |
| 19 | ZNF614 | zinc finger protein 614 | 142556 | 197619 | 256087 | 34, 4, 45 |
| 19 | ZNF615 | zinc finger protein 615 | 197619 | 256683 | 142556 | 42, 39, 9 |
| 19 | ZNF616 | zinc finger protein 616 | 204611 | 197608 | 196267 | |
| 19 | ZNF625 | zinc finger protein 625 | 257591 | 213297 | 196646 | 29, 24, 6 |
| 19 | ZNF626 | zinc finger protein 626 | 188171 | 237440 | 160229 | 34, 11, 42 |
| 19 | ZNF627 | zinc finger protein 627 | 198551 | 130165 | 102575 | |
| 19 | ZNF628 | zinc finger protein 628 | 197483 | 283567 | 090971 | 35, 4, 38 |
| 19 | ZNF649 | zinc finger protein 649 | 198093 | 283617 | 176024 | 13, 42, 1 |
| 19 | ZNF653 | zinc finger protein 653 | 161914 | 267477 | 130159 | 27, 29, 15 |
| 19 | ZNF66 | Zinc finger protein 66 | 160229 | 188171 | 105750 | 45, 37, 38 |
| 19 | ZNF665 | zinc finger protein 665 | 197497 | 197937 | 197928 | |
| 19 | ZNF667 | zinc finger protein 667 | 198046 | 198440 | 196263 | 31, 1, 25 |
| 19 | ZNF671 | zinc finger protein 671 | 083814 | 179909 | 152443 | 36, 39, 11 |
| 19 | ZNF675 | zinc finger protein 675 | 197372 | 167232 | 196172 | 30, 23, 13 |
| 19 | ZNF676 | zinc finger protein 676 | 196109 | 197134 | 196350 | 29, 4, 40 |
| 19 | ZNF677 | zinc finger protein 677 | 197928 | 197497 | 196131 | 2, 20, 33 |
| 19 | ZNF681 | zinc finger protein 681 | 196172 | 197372 | 213967 | 26, 45, 42 |
| 19 | ZNF682 | zinc finger protein 682 | 197124 | 184635 | 213988 | 15, 40, 7 |
| 19 | ZNF69 | zinc finger protein 69 | 198429 | 171291 | 196757 | 40, 7, 16 |
| 19 | ZNF699 | zinc finger protein 699 | 196110 | 237521 | 188321 | 31, 40, 5 |
| 19 | ZNF700 | zinc finger protein 700 | 196757 | 198429 | 267179 | 1, 13, 29 |
| 19 | ZNF701 | zinc finger protein 701 | 167562 | 198482 | 167766 | 40, 30, 21 |
| 19 | ZNF708 | zinc finger protein 708 | 182141 | 196705 | 172687 | 42, 30, 22 |
| 19 | ZNF709 | zinc finger protein 709 | 242852 | 269755 | 196826 | 2, 5, 20 |
| 19 | ZNF71 | zinc finger protein 71 | 197951 | 197016 | 268182 | 43, 26, 15 |
| 19 | ZNF714 | zinc finger protein 714 | 160352 | 118620 | 196705 | 40, 39, 25 |
| 19 | ZNF724 | Zinc finger protein 724 | 196081 | 183850 | 283201 | 36, 44, 17 |
| 19 | ZNF726 | zinc finger protein 726 | 213967 | 196172 | 213096 | 7, 27, 14 |
| 19 | ZNF728 | zinc finger protein 728 | 269067 | 213973 | 183850 | 7, 38, 37 |
| 19 | ZNF729 | zinc finger protein 729 | 196350 | 196109 | 197360 | 38, |
| 19 | ZNF730 | zinc finger protein 730 | 183850 | 269067 | 196081 | 38, 12, 39 |
| 19 | ZNF737 | zinc finger protein 737 | 237440 | 256229 | 188171 | 23, 41, 4 |
| 19 | ZNF738 | Zinc finger protein 738 | 172687 | 182141 | 196268 | 12, 21, 22 |
| 19 | ZNF749 | zinc finger protein 749 | 186230 | 186272 | 268163 | 35, 39, 30 |
| 19 | ZNF761 | zinc finger protein 761 | 160336 | 196417 | 198346 | 29, 18, 33 |
| 19 | ZNF763 | zinc finger protein 763 | 197054 | 267179 | 197647 | 36, 23, 6 |
| 19 | ZNF765 | zinc finger protein 765 | 196417 | 213799 | 160336 | 29, 21, 18 |
| 19 | ZNF766 | zinc finger protein 766 | 196214 | 105568 | 198464 | 4, 15, 43 |
| 19 | ZNF77 | zinc finger protein 77 | 175691 | 171970 | 104953 | 2, 27, 15 |
| 19 | ZNF772 | zinc finger protein 772 | 197128 | 178201 | 105136 | 31, 45, 36 |
| 19 | ZNF773 | zinc finger protein 773 | 152439 | 268107 | 121406 | 2, 25, 24 |
| 19 | ZNF776 | zinc finger protein 776 | 152443 | 083814 | 083828 | 39, 2, 22 |
| 19 | ZNF780A | zinc finger protein 780A | 197782 | 128000 | 130758 | 13, 4, 24 |
| 19 | ZNF780B | zinc finger protein 780B | 128000 | 187187 | 197782 | 26, 31, 32 |
| 19 | ZNF781 | zinc finger protein 781 | 196381 | 120784 | 267552 | 9, 45, 36 |
| 19 | ZNF784 | zinc finger protein 784 | 179922 | 261221 | 213015 | 31, 39, 4 |
| 19 | ZNF787 | zinc finger protein 787 | 142409 | 171487 | 167685 | 1, 27, 3 |
| 19 | ZNF790 | zinc finger protein 790 | 197863 | 267041 | 251247 | 23, 28, 30 |
| 19 | ZNF791 | zinc finger protein 791 | 173875 | 188033 | 269242 | 36, 45, 24 |
| 19 | ZNF792 | zinc finger protein 792 | 180884 | 168661 | 089351 | 26, 36, 22 |
| 19 | ZNF793 | zinc finger protein 793 | 188227 | 171827 | 171817 | 29, 13, 28 |
| 19 | ZNF799 | zinc finger protein 799 | 196466 | 268744 | 268870 | 3, 42, 36 |
| 19 | ZNF8 | zinc finger protein 8 | 278129 | 283515 | 267216 | |
| 19 | ZNF805 | zinc finger protein 805 | 204524 | 105146 | 197714 | 31, 15, 40 |
| 19 | ZNF808 | zinc finger protein 808 | 198482 | 258405 | 167562 | 21, 29, 39 |
| 19 | ZNF812P | Zinc finger protein 812, pseudogene | 224689 | 171466 | 196605 | 24, 18, 42 |
| 19 | ZNF813 | zinc finger protein 813 | 198346 | 160336 | 130844 | 16, 42, 24 |
| 19 | ZNF814 | zinc finger protein 814 | 204514 | 269343 | 198466 | 38, 10, |
| 19 | ZNF816 | zinc finger protein 816 | 180257 | 221874 | 269526 | 42, 40, 39 |
| 19 | ZNF823 | zinc finger protein 823 | 197933 | 102575 | 197044 | 45, 17, 15 |
| 19 | ZNF829 | zinc finger protein 829 | 185869 | 251247 | 198453 | 29, 4, 32 |
| 19 | ZNF83 | zinc finger protein 83 | 167766 | 167562 | 213020 | 26, 2, 24 |
| 19 | ZNF835 | zinc finger protein 835 | 127903 | 268182 | 269699 | 22, 17, 8 |
| 19 | ZNF836 | zinc finger protein 836 | 196267 | 204611 | 105568 | 1, 29, 2 |
| 19 | ZNF837 | zinc finger protein 837 | 152475 | 174586 | 083845 | 41, 2, 44 |
| 19 | ZNF841 | zinc finger protein 841 | 197608 | 256087 | 204611 | 30, 29, 33 |
| 19 | ZNF844 | zinc finger protein 844 | 223547 | 257446 | 214189 | 2, 42, 27 |
| 19 | ZNF845 | zinc finger protein 845 | 213799 | 203326 | 196417 | 41, 14, 4 |
| 19 | ZNF846 | zinc finger protein 846 | 196605 | 224689 | 127452 | |
| 19 | ZNF85 | zinc finger protein 85 | 105750 | 160229 | 118620 | 39, 38, 15 |
| 19 | ZNF850 | zinc finger protein 850 | 267041 | 189042 | 197863 | 32, 7, 6 |
| 19 | ZNF865 | zinc finger protein 865 | 261221 | 171443 | 179922 | 34, 29, 21 |
| 19 | ZNF878 | zinc finger protein 878 | 257446 | 257355 | 223547 | 28, |
| 19 | ZNF880 | zinc finger protein 880 | 221923 | 167554 | 167555 | 30, 29, 25 |
| 19 | ZNF90 | zinc finger protein 90 | 213988 | 197124 | 256229 | 18, 33, 12 |
| 19 | ZNF91 | zinc finger protein 91 | 167232 | 283201 | 197372 | |
| 19 | ZNF93 | zinc finger protein 93 | 184635 | 256771 | 197124 | |
| 19 | ZNF98 | zinc finger protein 98 | 197360 | 196350 | 229676 | |
| 19 | ZNF99 | zinc finger protein 99 | 213973 | 229676 | 269067 | 38, |
| 19 | ZIK1 | zinc finger protein interacting with K protein 1 | 171649 | 083817 | 183647 | 39, 22, 33 |
| 19 | ZFR2 | zinc finger RNA binding protein 2 | 105278 | 007264 | 167654 | 14, 12, 38 |
| 19 | ZSWIM4 | zinc finger SWIM-type containing 4 | 132003 | 280221 | 187556 | 40, 27, 33 |
| 19 | ZGLP1 | zinc finger, GATA-like protein 1 | 220201 | 105376 | 167807 | 3, 37, 28 |
| 19 | ZNF559-ZNF177 | ZNF559-ZNF177 readthrough | 270011 | 188321 | 188629 | |
| 19 | ZNF625-ZNF20 | ZNF625-ZNF20 readthrough (NMD candidate) | 213297 | 132010 | 257591 | |
| 19 | ZNF816-ZNF321P | ZNF816-ZNF321P readthrough | 221874 | 283088 | 180257 | 42, 23, 12 |

Table 9 provides the gene information for the genes found on Chromosomes 20-22, MT, X and Y.

TABLE 9
Genes on Chromosomes 20-22, MT, X and Y

| | | | | Up | Down | Tissue |
|---|---|---|---|---|---|---|
| Chr. | Gene Symbol | Gene Name | ENSG | ENSG | ENSG | Code |
| 20 | XRN2 | 5′-3′ exoribonuclease 2 | 088930 | 088970 | 125816 | 42, 43, 18 |
| 20 | AAR2 | AAR2 splicing factor homolog | 131043 | 088367 | 080845 | 9, 35, 2 |
| 20 | ABHD12 | abhydrolase domain containing 12 | 100997 | 100994 | 101003 | 26, 27, 34 |
| 20 | ABHD16B | abhydrolase domain containing 16B | 183260 | 130584 | 101150 | 38, 7, 23 |
| 20 | ACTL10 | actin like 10 | 182584 | 149609 | 101412 | 33, 36, 16 |
| 20 | ADNP | activity dependent neuroprotector homeobox | 101126 | 124243 | 000419 | 22, 3, 18 |
| 20 | ACSS1 | acyl-CoA synthetase short-chain family member 1 | 154930 | 101474 | 100987 | 7, 26, 24 |
| 20 | ACSS2 | acyl-CoA synthetase short-chain family member 2 | 131069 | 131067 | 100983 | 21, 24, 2 |
| 20 | ACOT8 | acyl-CoA thioesterase 8 | 101473 | 124104 | 132801 | 39, 29, 22 |
| 20 | ADAM33 | ADAM metallopeptidase domain 33 | 149451 | 125861 | 088827 | 44, 1, 42 |
| 20 | AP5S1 | adaptor related protein complex 5 sigma 1 subunit | 125843 | 101224 | 088888 | 3, 40, 20 |
| 20 | ASXL1 | additional sex combs like 1, transcriptional regulator | 171456 | 101350 | 197183 | 22, 29, 40 |
| 20 | ADA | adenosine deaminase | 196839 | 168734 | 064205 | 5, 22, 43 |
| 20 | AHCY | adenosylhomocysteinase | 101444 | 101440 | 078747 | 13, 21, 7 |
| 20 | ADRM1 | adhesion regulating molecule 1 | 130706 | 130703 | 130702 | 35, 27, 9 |
| 20 | APMAP | adipocyte plasma membrane associated protein | 101474 | 077984 | 154930 | 40, 1, 5 |
| 20 | ADIG | adipogenin | 182035 | 170471 | 124143 | 38 |
| 20 | ARFGAP1 | ADP ribosylation factor GTPase activating protein 1 | 101199 | 101198 | 101203 | |
| 20 | ARFGEF2 | ADP ribosylation factor guanine nucleotide exchange factor 2 | 124198 | 124126 | 124207 | 1, 29, 9 |
| 20 | ARFRP1 | ADP ribosylation factor related protein 1 | 101246 | 243509 | 197114 | |
| 20 | ADRA1D | adrenoceptor alpha 1D | 171873 | 088826 | 171867 | 6, 23, 33 |
| 20 | ASIP | agouti signaling protein | 101440 | 125977 | 101444 | 15 |
| 20 | TTPAL | alpha tocopherol transfer protein like | 124120 | 101076 | 132824 | 11, 29, 24 |
| 20 | NPEPL1 | aminopeptidase-like 1 | 215440 | 254995 | 087460 | 10, 16, 12 |
| 20 | ANGPT4 | angiopoietin 4 | 101280 | 125898 | 101282 | 38, 44, 32 |
| 20 | ANKEF1 | ankyrin repeat and EF-hand domain containing 1 | 132623 | 101349 | 132639 | 38, 27, 42 |
| 20 | ANKRD60 | ankyrin repeat domain 60 | 124227 | 124237 | 124209 | |
| 20 | APCDD1L | APC down-regulated 1 like | 198768 | 124164 | 124222 | 13, 10, 6 |
| 20 | AVP | arginine vasopressin | 101200 | 101405 | 185019 | 17, 8 |
| 20 | ACTR5 | ARP5 actin-related protein 5 homolog | 101442 | 101438 | 101445 | 3, 27, 38 |
| 20 | ATP5E | ATP synthase, H+ transporting, mitochondrial F1 complex, epsilon subunit | 124172 | 101162 | 101166 | 41, 19, 31 |
| 20 | ATP9A | ATPase phospholipid transporting 9A (putative) | 054793 | 101096 | 101115 | 26, 31, 18 |
| 20 | ATRN | attractin | 088812 | 088854 | 125861 | 1, 30, 40 |
| 20 | AURKA | aurora kinase A | 087586 | 124098 | 101138 | 13, 9, 6 |
| 20 | BPI | bactericidal/permeability-increasing protein | 101425 | 149633 | 129988 | |
| 20 | BIRC7 | baculoviral IAP repeat containing 7 | 101197 | 149658 | 101198 | 35, 14, 12 |
| 20 | BANF2 | barrier to autointegration factor 2 | 125888 | 125844 | 089006 | 11, 38, 10 |
| 20 | BHLHE23 | basic helix-loop-helix family member e23 | 125533 | 101194 | 149658 | |
| 20 | BCL2L1 | BCL2 like 1 | 171552 | 131055 | 088325 | 19, 37, 27 |
| 20 | BFSP1 | beaded filament structural protein 1 | 125864 | 125851 | 283253 | 14, 19, 6 |
| 20 | B4GALT5 | beta-1,4-galactosyltransferase 5 | 158470 | 124212 | 197818 | 27, 6, 42 |
| 20 | BLCAP | bladder cancer associated protein | 166619 | 197122 | 053438 | |
| 20 | BMP2 | bone morphogenetic protein 2 | 125845 | 101311 | 101323 | 18, 9, 15 |
| 20 | BMP7 | bone morphogenetic protein 7 | 101144 | 087510 | 054796 | 15, 21, 14 |
| 20 | BPIFA1 | BPI fold containing family A member 1 | 198183 | 131059 | 125999 | 30, 18, 20 |
| 20 | BPIFA2 | BPI fold containing family A member 2 | 131050 | 186191 | 131059 | 34, 25, 23 |
| 20 | BPIFA3 | BPI fold containing family A member 3 | 131059 | 131050 | 198183 | 38, 14, 8 |
| 20 | BPIFB1 | BPI fold containing family B member 1 | 125999 | 198183 | 101391 | 30, 18, 23 |
| 20 | BPIFB2 | BPI fold containing family B member 2 | 078898 | 167098 | 167104 | 42, 34, 18 |
| 20 | BPIFB3 | BPI fold containing family B member 3 | 186190 | 167104 | 186191 | |
| 20 | BPIFB4 | BPI fold containing family B member 4 | 186191 | 186190 | 131050 | 30, 19, 6 |
| 20 | BPIFB6 | BPI fold containing family B member 6 | 167104 | 078898 | 186190 | |
| 20 | BCAS1 | breast carcinoma amplified sequence 1 | 064787 | 171940 | 019186 | 8, 42, 4 |
| 20 | BCAS4 | breast carcinoma amplified sequence 4 | 124243 | 124171 | 101126 | 22, 21, 4 |
| 20 | BTBD3 | BTB domain containing 3 | 132640 | 101384 | 172296 | 8, 37, 11 |
| 20 | CDH22 | cadherin 22 | 149654 | 101017 | 080189 | 26, 28, 18 |
| 20 | CDH26 | cadherin 26 | 124215 | 132825 | 179242 | 42, 24, 4 |
| 20 | CDH4 | cadherin 4 | 179242 | 124215 | 130699 | 14, 8, 12 |
| 20 | PKIG | cAMP-dependent protein kinase inhibitor gamma | 168734 | 132824 | 196839 | 29, 41, 18 |
| 20 | CPXM1 | carboxypeptidase X, M14 family member 1 | 088882 | 088881 | 241690 | 34, 12, 44 |
| 20 | CRLS1 | cardiolipin synthase 1 | 088766 | 125885 | 125872 | 19, 17, 20 |
| 20 | CASS4 | Cas scaffolding protein family member 4 | 087589 | 101138 | 022277 | 5, 32, 37 |
| 20 | CSNK2A1 | casein kinase 2 alpha 1 | 101266 | 125875 | 125878 | 24, 39, 13 |
| 20 | CTNNBL1 | catenin beta like 1 | 132792 | 053438 | 132821 | 13, 21, 41 |
| 20 | CTSA | cathepsin A | 064601 | 124257 | 100979 | 2, 35, 16 |
| 20 | CTSZ | cathepsin Z | 101160 | 101158 | 101162 | 37, 16, 23 |
| 20 | CBFA2T2 | CBFA2/RUNX1 translocation partner 2 | 078699 | 101400 | 125967 | 37, 33, 27 |
| 20 | CEBPB | CCAAT/enhancer binding protein beta | 172216 | 240849 | 196396 | 26, 3, 21 |
| 20 | CTCFL | CCCTC-binding factor like | 124092 | 132819 | 124253 | 34, 7, 38 |
| 20 | CCM2L | COM2 like scaffolding protein | 101331 | 260903 | 101336 | 36, 1, 26 |
| 20 | CD40 | CD40 molecule | 101017 | 124160 | 149654 | 22, 21, 41 |
| 20 | CD93 | CD93 molecule | 125810 | 178726 | 132661 | 43, 1, 32 |
| 20 | CABLES2 | Cdk5 and Abl enzyme substrate 2 | 149679 | 171858 | 130701 | 38, 18, 32 |
| 20 | CDK5RAP1 | CDK5 regulatory subunit associated protein 1 | 101391 | 125999 | 101400 | 3, 36, 20 |
| 20 | CDS2 | CDP-diacylglycerol synthase 2 | 101290 | 132646 | 101292 | 13, 26, 38 |
| 20 | CDC25B | cell division cycle 25B | 101224 | 125817 | 125843 | 34, 39, 3 |
| 20 | CENPB | centromere protein B | 125817 | 101222 | 101224 | 34, 35, 14 |
| 20 | CEP250 | centrosomal protein 250 | 126001 | 125965 | 125975 | 22, 16, 18 |
| 20 | CBLN4 | cerebellin 4 precursor | 054803 | 101134 | 124089 | 2, 8, 39 |
| 20 | CHMP4B | charged multivesicular body protein 4B | 101421 | 131061 | 125970 | 16, 18, 11 |
| 20 | CHRNA4 | cholinergic receptor nicotinic alpha 4 subunit | 101204 | 101203 | 075043 | |
| 20 | CHD6 | chromodomain helicase DNA binding protein 6 | 124177 | 183798 | 196090 | 25, 23, 11 |
| 20 | CHGB | chromogranin B | 089199 | 171984 | 089195 | 31, 2, 28 |
| 20 | C20orf141 | chromosome 20 open reading frame 141 | 258713 | 241690 | 198326 | 38, 14, 32 |
| 20 | C20orf144 | chromosome 20 open reading frame 144 | 149609 | 125967 | 182584 | 38, 32, 39 |
| 20 | C20orf173 | chromosome 20 open reading frame 173 | 125975 | 126001 | 125991 | 38, 5, |
| 20 | C20orf194 | chromosome 20 open reading frame 194 | 088854 | 088836 | 088812 | 1, 40, 18 |
| 20 | C20orf196 | chromosome 20 open reading frame 196 | 171984 | 125772 | 089199 | 10, 30, 22 |
| 20 | C20orf202 | chromosome 20 open reading frame 202 | 215595 | 125895 | 244588 | 20, 32, 10 |
| 20 | C20orf24 | chromosome 20 open reading frame 24 | 101084 | 259399 | 101082 | 34, 2, 29 |
| 20 | C20orf27 | chromosome 20 open reading frame 27 | 101220 | 132622 | 101222 | 3, 27, 26 |
| 20 | C20orf85 | chromosome 20 open reading frame 85 | 124237 | 124225 | 124227 | 30, 38, 20 |
| 20 | C20orf96 | chromosome 20 open reading frame 96 | 196476 | 186458 | 247315 | 40, 39, 36 |
| 20 | CSE1L | chromosome segregation 1 like | 124207 | 124198 | 124214 | 38, 42, 24 |
| 20 | CFAP61 | cilia and flagella associated protein 61 | 089101 | 101343 | 173404 | 38, 42, 15 |
| 20 | CSTF1 | cleavage stimulation factor subunit 1 | 101138 | 087586 | 087589 | 13, 2, 5 |
| 20 | COL9A3 | collagen type IX alpha 3 chain | 092758 | 060491 | 101190 | 24, 11, 41 |
| 20 | COL20A1 | collagen type XX alpha 1 chain | 101203 | 101199 | 101204 | 8, 14, 38 |
| 20 | COMMD7 | COMM domain containing 7 | 149600 | 283233 | 088305 | 43, 34, 41 |
| 20 | CPNE1 | copine 1 | 214078 | 061656 | 272897 | 41, 21, 27 |
| 20 | CRNKL1 | crooked neck pre-mRNA splicing factor 1 | 101343 | 173418 | 089101 | 34, 42, 13 |
| 20 | CNBD2 | cyclic nucleotide binding domain containing 2 | 149646 | 171222 | 088367 | 38, 10, 3 |
| 20 | CST11 | cystatin 11 | 125831 | 125823 | 125815 | 38, |
| 20 | CST8 | cystatin 8 | 125815 | 125831 | 101435 | 38, |
| 20 | CST9 | cystatin 9 | 173335 | 101435 | 101439 | 23, |
| 20 | CST9L | cystatin 9-like | 101435 | 125815 | 173335 | 38, 32, |
| 20 | CST3 | cystatin C | 101439 | 173335 | 101441 | 29, 37, 8 |
| 20 | CST5 | cystatin D | 170367 | 170369 | 149435 | 34, 23, |
| 20 | CST7 | cystatin F | 077984 | 101463 | 101474 | 5, 1, 36 |
| 20 | CSTL1 | cystatin like 1 | 125823 | 125814 | 125831 | 42, 38, |
| 20 | CST4 | cystatin S | 101441 | 101439 | 170373 | 34, 14, 25 |
| 20 | CST2 | cystatin SA | 170369 | 170373 | 170367 | 34, 28, 37 |
| 20 | CST1 | cystatin SN | 170373 | 101441 | 170369 | 34, 4, 16 |
| 20 | COX4I2 | cytochrome c oxidase subunit 4I2 | 131055 | 125968 | 171552 | 32, 25, 17 |
| 20 | CYP24A1 | cytochrome P450 family 24 subfamily A member 1 | 019186 | 064787 | 101132 | 11, 33, 18 |
| 20 | DDRGK1 | DDRGK domain containing 1 | 198171 | 088899 | 125877 | 41, 27, 21 |
| 20 | DDX27 | DEAD-box helicase 27 | 124228 | 124214 | 124201 | 13, 21, 3 |
| 20 | DHX35 | DEAH-box helicase 35 | 101452 | 101447 | 204103 | 24, 39, 42 |
| 20 | DIDO1 | death inducer-obliterator 1 | 101191 | 101190 | 101193 | 25, 39, 24 |
| 20 | DEFB115 | defensin beta 115 | 215547 | 175170 | 215545 | |
| 20 | DEFB116 | defensin beta 116 | 215545 | 215547 | 131068 | |
| 20 | DEFB118 | defensin beta 118 | 131068 | 215545 | 180483 | 38, |
| 20 | DEFB119 | defensin beta 119 | 180483 | 131068 | 204548 | |
| 20 | DEFB121 | defensin beta 121 | 204548 | 180483 | 180424 | |
| 20 | DEFB123 | defensin beta 123 | 180424 | 204548 | 180383 | 38, |
| 20 | DEFB124 | defensin beta 124 | 180383 | 180424 | 088320 | |
| 20 | DEFB125 | defensin beta 125 | 178591 | 124209 | 125788 | 38, |
| 20 | DEFB126 | defensin beta 126 | 125788 | 178591 | 088782 | 38, |
| 20 | DEFB127 | defensin beta 127 | 088782 | 125788 | 185982 | 38, |
| 20 | DEFB128 | defensin beta 128 | 185982 | 088782 | 125903 | |
| 20 | DEFB129 | defensin beta 129 | 125903 | 185982 | 186458 | 38, |
| 20 | DEFB132 | defensin beta 132 | 186458 | 125903 | 196476 | 33, |
| 20 | DNTTIP1 | deoxynucleotidyltransferase terminal interacting protein 1 | 101457 | 124116 | 175063 | 33, 37, 9 |
| 20 | DSTN | destrin, actin depolymerizing factor | 125868 | 283253 | 125844 | 43, 13, 26 |
| 20 | DLGAP4 | DLG associated protein 4 | 080845 | 131043 | 101335 | 35, 28, 3 |
| 20 | DNMT3B | DNA methyltransferase 3 beta | 088305 | 149600 | 101367 | |
| 20 | DNAJC5 | DnaJ heat shock protein family (Hsp40) member C5 | 101152 | 101150 | 198276 | 14, 16, 23 |
| 20 | DOK5 | docking protein 5 | 101134 | 280041 | 054803 | 27, 8, 1 |
| 20 | DPM1 | dolichyl-phosphate mannosyltransferase subunit 1, catalytic | 000419 | 101126 | 124217 | 4, 34, 10 |
| 20 | DZANK1 | double zinc ribbon and ankyrin repeat domains 1 | 089091 | 125846 | 132664 | 29, 26, 3 |
| 20 | DSN1 | DSN1 homolog, MIS12 kinetochore complex component | 149636 | 101079 | 149639 | 13, 21, 1 |
| 20 | DTD1 | D-tyrosyl-tRNA deacylase 1 | 125821 | 232388 | 132631 | 10, 31, 41 |
| 20 | DUSP15 | dual specificity phosphatase 15 | 149599 | 179772 | 131044 | 26, 38, 17 |
| 20 | DYNLRB1 | dynein light chain roadblock-type 1 | 125971 | 283472 | 101460 | 29, 27, 43 |
| 20 | DBNDD2 | dysbindin domain containing 2 | 244274 | 124251 | 124155 | 26, 1, 16 |
| 20 | E2F1 | E2F transcription factor 1 | 101412 | 182584 | 101417 | 9, 3, 34 |
| 20 | EBF4 | early B-cell factor 4 | 088881 | 101365 | 088882 | 26, 34, 18 |
| 20 | ENTPD6 | ectonucleoside triphosphate diphosphohydrolase 6 (putative) | 197586 | 100987 | 100994 | 3, 31, 39 |
| 20 | EFCAB8 | EF-hand calcium binding domain 8 | 215529 | 101367 | 167098 | 38, |
| 20 | EMILIN3 | elastin microfibril interfacer 3 | 183798 | 132793 | 124177 | 15, 27, 14 |
| 20 | EDN3 | endothelin 3 | 124205 | 124203 | 087495 | 11, 26, 18 |
| 20 | ELMO2 | engulfment and cell motility 2 | 062598 | 080189 | 198185 | 8, 21, 38 |
| 20 | EPPIN | epididymal peptidase inhibitor | 101448 | 249139 | 158901 | 38, 15, 44 |
| 20 | EPPIN-WFDC6 | EPPIN-WFDC6 readthrough | 249139 | 243543 | 101448 | |
| 20 | EDEM2 | ER degradation enhancing alpha-mannosidase like protein 2 | 088298 | 100991 | 101000 | 21, 36, 5 |
| 20 | ERGIC3 | ERGIC and golgi 3 | 125991 | 125975 | 061656 | 40, 16, 27 |
| 20 | EPB41L1 | erythrocyte membrane protein band 4.1 like 1 | 088367 | 149646 | 131043 | 18, 27, 26 |
| 20 | ESF1 | ESF1 nucleolar pre-rRNA processing protein hemeleg | 089048 | 089123 | 101247 | 19, 1, 12 |
| 20 | EEF1A2 | eukaryotic translation elongation factor 1 alpha 2 | 101210 | 075043 | 125534 | 23, 26, 28 |
| 20 | EIF2S2 | eukaryotic translation initiation factor 2 subunit beta | 125977 | 125970 | 101440 | 3, 13, 6 |
| 20 | EIF6 | eukaryotic translation initiation factor 6 | 242372 | 261582 | 125998 | 21, 35, 34 |
| 20 | EYA2 | EYA transcriptipnal coactivator and phosphatase 2 | 064655 | 197496 | 101040 | |
| 20 | FAM110A | family with sequence similarity 110 member A | 125898 | 101276 | 101280 | 5, 25, 35 |
| 20 | FAM182B | Family with sequence similarity 182 member B | 175170 | 130684 | 215547 | 38, 15, 8 |
| 20 | FAM209A | family with sequence similarity 209 member A | 124103 | 124091 | 213714 | 38, 15, 32 |
| 20 | FAM209B | family with sequence similarity 209 member B | 213714 | 124103 | 087510 | 38, 5, 32 |
| 20 | FAM210B | family with sequence similarity 210 member B | 124098 | 124089 | 087586 | 33, 35, 43 |
| 20 | FAM217B | family with sequence similarity 217 member B | 196227 | 196074 | 132825 | 11, 4, 22 |
| 20 | FAM65C | family with sequence similarity 65 member C | 042062 | 196396 | 124171 | 2, 1, 17 |
| 20 | FAM83C | family with sequence similarity 83 member C | 125998 | 242372 | 101019 | 24, 18, 38 |
| 20 | FAM83D | family with sequence similarity 83 member D | 101447 | 101445 | 101452 | 9, 21, 27 |
| 20 | FASTKD5 | FAST kinase domains 5 | 215251 | 185019 | 088899 | 21, 3, 11 |
| 20 | FITM2 | fat storage inducing transmembrane protein 2 | 197296 | 124194 | 101074 | 21, 27, 25 |
| 20 | FERMT1 | fermitin family member 1 | 101311 | 125872 | 125845 | 16, 4, 30 |
| 20 | FLRT3 | fibronectin leucine rich transmembrane protein 3 | 125848 | 172264 | 089177 | 11, 29, 12 |
| 20 | FNDC11 | fibronectin type III demain containing 11 | 125531 | 125508 | 130589 | 9, 38, 32 |
| 20 | FKBP1A | FK506 binding protein 1A | 088832 | 274322 | 088833 | 34, 35, 21 |
| 20 | FOXA2 | forkhead boxA2 | 125798 | 125813 | 132671 | 20, 16, 28 |
| 20 | FOXS1 | forkhead box S1 | 179772 | 101306 | 149599 | 25, 33, 32 |
| 20 | GTSF1L | gametocyte specific factor 1 like | 124196 | 101057 | 124191 | 38, 32, 20 |
| 20 | GGT7 | gamma-glutamyltransferase 7 | 131067 | 078804 | 131069 | 18, 20, 33 |
| 20 | GGTLC1 | gamma-glutamyltransferase light chain 1 | 149435 | 170367 | 101463 | 5, 20, |
| 20 | GDAP1L1 | ganglipside induced differentiation associated protein 1 like 1 | 124194 | 132823 | 197296 | 8, 14, 15 |
| 20 | GATA5 | GATA binding protein 5 | 130700 | 130701 | 101187 | 44, 20, 17 |
| 20 | GFRA4 | GDNF family receptor alpha 4 | 125861 | 088812 | 149451 | |
| 20 | GZF1 | GDNF inducible zinc finger protein 1 | 125812 | 132661 | 125814 | |
| 20 | GID8 | GID complex subunit 8 homolog | 101193 | 101191 | 101194 | 11, 6, 42 |
| 20 | GINS1 | GINS complex subunit 1 | 101003 | 100997 | 101004 | 9, 12, 21 |
| 20 | GMEB2 | glucocorticoid modulatory element binding protein 2 | 101216 | 130589 | 197457 | 41, 21, 1 |
| 20 | GCNT7 | glucosaminyl (N-acetyl) transferase family member 7 | 124091 | 022277 | 124103 | 1, 38, 28 |
| 20 | GSS | glutathione synthetase | 100983 | 131069 | 078814 | 24, 16, 3 |
| 20 | GPCPD1 | glycerophosphocholine phosphodiesterase 1 | 125772 | 101292 | 171984 | 44, 26, 18 |
| 20 | PYGB | glycogen phosphorylase B | 100994 | 197586 | 100997 | 16, 26, 35 |
| 20 | GNAS | GNAS complex locus | 087460 | 215440 | 101158 | 31, 40, 18 |
| 20 | GNRH2 | gonadotropin releasing hormone 2 | 125787 | 132670 | 125901 | 14, |
| 20 | GDF5 | growth differentiation factor 5 | 125965 | 204183 | 126001 | 34, 31, 6 |
| 20 | GDF5OS | Growth differentiation factor 5 opposite strand | 204183 | 101019 | 125965 | 27, 14, 23 |
| 20 | GHRH | growth hormone releasing hormone | 118702 | 118705 | 101363 | 20, |
| 20 | HCK | HCK proto-oncogene, Src family tyrosine kinase | 101336 | 101331 | 101337 | 5, 36, 21 |
| 20 | HSPA12B | heat shock protein family A (Hsp70) member 12B | 132622 | 088827 | 101220 | 36, 43, 15 |
| 20 | HELZ2 | helicase with zinc finger 2 | 130589 | 125531 | 101216 | 22, 1, 23 |
| 20 | HNF4A | hepatocyte nuclear factor 4 alpha | 101076 | 101074 | 124120 | 16, 17, 37 |
| 20 | HRH3 | histamine receptor H3 | 101180 | 101181 | 130703 | 8, 12, 23 |
| 20 | HM13 | histocompatibility minor 13 | 101294 | 088320 | 125968 | 27, 13, 33 |
| 20 | HAO1 | hydroxyacid oxidase 1 | 101323 | 125845 | 125827 | 19, 28, 17 |
| 20 | ID1 | inhibitor of DNA binding 1, HLH protein | 125968 | 101294 | 131055 | 37, 3, 6 |
| 20 | ITPA | inosine triphosphatase | 125877 | 198171 | 088836 | 15, 1, 3 |
| 20 | INSM1 | INSM transcriptional repressor 1 | 173404 | 089101 | 188559 | 28, 20, 31 |
| 20 | IFT52 | intraflagellar transport 52 | 101052 | 101049 | 101057 | 26, 5, 29 |
| 20 | IDH3B | isocitrate dehydrogenase 3 (NAD(+)) beta | 101365 | 101361 | 088881 | 18, 3, 41 |
| 20 | ISM1 | isthmin 1 | 101230 | 172296 | 089123 | 1, 2, 42 |
| 20 | ITCH | itchy E3 ubiquitin protein ligase | 078747 | 101444 | 283472 | 13, 42, 24 |
| 20 | ITCH | itchy E3 ubiquitin protein ligase | 283472 | 078747 | 125971 | 13, 42, 24 |
| 20 | JAG1 | jagged 1 | 101384 | 149346 | 132640 | 18, 24, 26 |
| 20 | JPH2 | junctophilin 2 | 149596 | 124191 | 132823 | 44, 38, 15 |
| 20 | KIAA1755 | KIAA1755 | 149633 | 198959 | 101425 | |
| 20 | KIF16B | kinesin family member 16B | 089177 | 125848 | 280240 | 2, 24, 29 |
| 20 | KIF3B | kinesin family member 3B | 101350 | 101346 | 171456 | 29, 22, 33 |
| 20 | KIZ | kizuna centrosomal protein | 088970 | 188559 | 088930 | |
| 20 | L3MBTL1 | l(3)mbt-like 1 (Drosophila) | 185513 | 124193 | 277611 | 11, 42, 40 |
| 20 | LAMA5 | laminin subunit alpha 5 | 130702 | 130706 | 171858 | 13, 3, 26 |
| 20 | LIME1 | Lck interacting transmembrane adaptor 1 | 203896 | 273154 | 273047 | 37, 17, 5 |
| 20 | LRRN4 | leucine rich repeat neuronal 4 | 125872 | 088766 | 101311 | 17, 12, 27 |
| 20 | LZTS3 | leucine zipper tumor suppressor family member 3 | 088899 | 215251 | 198171 | 14, 11, 2 |
| 20 | LPIN3 | lipin 3 | 132793 | 174306 | 183798 | 3, 40, 18 |
| 20 | LBP | lipopolysaccharide binding protein | 129988 | 101425 | 170471 | 19, 34, 5 |
| 20 | LKAAEAR1 | LKAAEAR motif containing 1 | 171695 | 125510 | 125522 | 38, |
| 20 | LINC00176 | Long intergenic non-protein coding RNA 176 | 196421 | 101161 | 203883 | 27, 5, 39 |
| 20 | LINC00493 | Long intergenic non-protein coding RNA 493 | 232388 | 101310 | 125821 | 18, 25, 11 |
| 20 | LSM14B | LSM family member 14B | 149657 | 130699 | 101182 | 33, 23, 9 |
| 20 | KAT14 | lysine acetyltransferase 14 | 149474 | 232838 | 125846 | 1, 31, 33 |
| 20 | LAMP5 | lysosomal associated membrane protein family member 5 | 125869 | 101333 | 101349 | 31, 8, 18 |
| 20 | MACROD2 | MACRO domain containing 2 | 172264 | 101251 | 125848 | |
| 20 | MROH8 | maestro heat like repeat family member 8 | 101353 | 080839 | 118705 | 38, 9, 39 |
| 20 | MAFB | MAF bZIP transcription factor B | 204103 | 101452 | 198900 | 36, 24, 29 |
| 20 | MANBAL | mannosidase beta like | 101363 | 118702 | 197122 | 26, 37, 27 |
| 20 | MATN4 | matrilin 4 | 124159 | 124107 | 124232 | 12, 4, 2 |
| 20 | MMP24 | matrix metallopeptidase 24 | 125966 | 126005 | 261582 | 26, 17, 1 |
| 20 | MMP9 | matrix metallopeptidase 9 | 100985 | 198026 | 124140 | 5, 30, 36 |
| 20 | MKKS | McKusick-Kaufman syndrome | 125863 | 132639 | 149346 | |
| 20 | MC3R | melanocortin 3 receptor | 124089 | 054803 | 124098 | |
| 20 | MAP1LC3A | microtubule associated protein 1 light chain 3 alpha | 101460 | 125971 | 101464 | 30, 26, 14 |
| 20 | MAPRE1 | microtubule associated protein RP/EB family member 1 | 101367 | 088305 | 215529 | 43, 11, 45 |
| 20 | MCM8 | minichromosome maintenance 8 homologous recombination repair factor | 125885 | 089195 | 088766 | 21, 35, 3 |
| 20 | MAVS | mitochondrial antiviral signaling protein | 088888 | 125843 | 125779 | 26, 39, 23 |
| 20 | MGME1 | mitochondrial genome maintenance exonuclease 1 | 125871 | 125850 | 232838 | 41, 11, 13 |
| 20 | MRPS26 | mitochondrial ribosomal protein S26 | 125901 | 125787 | 101405 | 29, 22, 27 |
| 20 | MTG2 | mitochondrial ribosome associated GTPase 2 | 101181 | 184402 | 101180 | |
| 20 | MMP24-AS1 | MMP24 antisense RNA 1 | 126005 | 101000 | 125966 | |
| 20 | MOCS3 | molybdenum cofactor synthesis 3 | 124217 | 000419 | 026559 | 12, 2, 9 |
| 20 | MRGBP | MRG domain binding protein | 101189 | 101188 | 060491 | 38, 9, 29 |
| 20 | MTRNR2L3 | MT-RNR2-like 3 | 256222 | 101146 | 132819 | |
| 20 | MYBL2 | MYB proto-oncogene like 2 | 101057 | 101052 | 124196 | 41, 21, 9 |
| 20 | MYT1 | myelin transcription factor 1 | 196132 | 125522 | 203880 | 26, 31, 29 |
| 20 | MYH7B | myosin heavy chain 7B | 078814 | 100983 | 100991 | 10, 38, 15 |
| 20 | MYL9 | myosin light chain 9 | 101335 | 080845 | 118707 | 43, 6, 1 |
| 20 | MYLK2 | myosin light chain kinase 2 | 101306 | 088325 | 179772 | 25, 26, 38 |
| 20 | NAA20 | N(alpha)-acetyltransferase 20, NatB catalytic subunit | 173418 | 132669 | 101343 | 28, 22, 30 |
| 20 | NANP | N-acetylneuraminic acid phosphatase | 170191 | 101004 | 130684 | 24, 39, 40 |
| 20 | NDUFAF5 | NADH:ubiquinone oxidoreductase complex assembly factor 5 | 101247 | 089048 | 101251 | 29, 41, 22 |
| 20 | NDRG3 | NDRG family member 3 | 101079 | 101082 | 149636 | 29, 23, 38 |
| 20 | NELFCD | negative elongation factor complex member C/D | 101158 | 087460 | 101160 | 34, 3, 1 |
| 20 | NEURL2 | neuralized E3 ubiquitin protein ligase 2 | 124257 | 149634 | 064601 | |
| 20 | NRSN2 | neurensin 2 | 125841 | 177732 | 101255 | 2, 3, 8 |
| 20 | NNAT | neuronatin | 053438 | 166619 | 132792 | 31, 45, 14 |
| 20 | NPBWR2 | neuropeptides B and W receptor 2 | 125522 | 171695 | 196132 | |
| 20 | NTSR1 | neurotensin receptor 1 | 101188 | 101187 | 101189 | 3, 28, 16 |
| 20 | NFS1 | NFS1, cysteine desulfurase | 244005 | 244462 | 125995 | 2, 13, 29 |
| 20 | NINL | ninein like | 101004 | 101003 | 170191 | 26, 29, 1 |
| 20 | NKX2-2 | NK2 homeobox 2 | 125820 | 125816 | 125813 | 28, 6, 8 |
| 20 | NKX2-4 | NK2 homeobox 4 | 125816 | 088930 | 125820 | 38, |
| 20 | NOP56 | NOP56 ribonucleoprotein | 101361 | 149488 | 101365 | 13, 3, 17 |
| 20 | NAPB | NSF attachment protein beta | 125814 | 125812 | 125823 | 8, 42, 4 |
| 20 | NSFL1C | NSFL1 cofactor | 088833 | 088832 | 196209 | 23, 41, 32 |
| 20 | NECAB3 | N-terminal EF-hand calcium binding protein 3 | 125967 | 078699 | 149609 | 9, 6, 27 |
| 20 | NFATC2 | nuclear factor of activated T-cells 2 | 101096 | 026559 | 054793 | 22, 36, 29 |
| 20 | NCOA3 | nuclear receptor coactivator 3 | 124151 | 101040 | 196562 | 13, 22, 23 |
| 20 | NCOA5 | nuclear receptor coactivator 5 | 124160 | 124140 | 101017 | 41, 40, 22 |
| 20 | NCOA6 | nuclear receptor coactivator 6 | 198646 | 101464 | 078804 | |
| 20 | NXT1 | nuclear transport factor 2 like export factor 1 | 132661 | 125810 | 125812 | 35, 3, 44 |
| 20 | NOL4L | nucleolar protein 4 like | 197183 | 171456 | 283233 | 26, 27, 38 |
| 20 | OGFR | opioid growth factor receptor | 060491 | 101189 | 092758 | 35, 13, 41 |
| 20 | OPRL1 | opioid related nociceptin receptor 1 | 125510 | 171700 | 171695 | 26, 21, 8 |
| 20 | OCSTAMP | osteoclast stimulatory transmembrane protein | 149635 | 198185 | 158296 | 5, |
| 20 | OTOR | otoraplin | 125879 | 125870 | 125851 | 11, 8, |
| 20 | OVOL2 | ovo like zinc finger 2 | 125850 | 089006 | 125871 | 16, 27, 20 |
| 20 | OSER1 | oxidative stress responsive serine rich 1 | 132823 | 149596 | 124194 | |
| 20 | OSBPL2 | oxysterol binding protein like 2 | 130703 | 101180 | 130706 | |
| 20 | OXT | oxytocin/neurophysin I prepropeptide | 101405 | 125901 | 101200 | 17, 8, |
| 20 | PAK5 | p21 (RAC1) activated kinase 5 | 101349 | 125869 | 132623 | 8, 14, 24 |
| 20 | PDRG1 | p53 and DNA damage regulated 1 | 088356 | 131044 | 260903 | 9, 1, 3 |
| 20 | PAX1 | paired box 1 | 125813 | 125820 | 125798 | 29, 39, 15 |
| 20 | PPDPF | pancreatic progenitor cell differentiation and proliferation factor | 125534 | 101210 | 101213 | 34, 23, 27 |
| 20 | PANK2 | pantothenate kinase 2 | 125779 | 088888 | 101236 | 40, 22, 18 |
| 20 | PARD6B | par-6 family cell polarity regulator beta | 124171 | 042062 | 124243 | 40, 23, 14 |
| 20 | PCED1A | PC-esterase domain containing 1A | 132635 | 198326 | 215305 | 4, 26, 13 |
| 20 | PCIF1 | PDX1 C-terminal inhibiting factor 1 | 100982 | 100979 | 198026 | 41, 25, 14 |
| 20 | PI3 | peptidase inhibitor 3 | 124102 | 168703 | 124233 | 18, 30, 24 |
| 20 | PXMP4 | peroxisomal membrane protein 4 | 101417 | 101412 | 131061 | 13, 40, 14 |
| 20 | PET117 | PET117 homolog | 232838 | 125871 | 149474 | 29, 28, 14 |
| 20 | PHF20 | PHD finger protein 20 | 025293 | 131051 | 171222 | 12, 45, 1 |
| 20 | PHACTR3 | phosphatase and actin regulator 3 | 087495 | 124205 | 196074 | |
| 20 | PIGT | phosphatidylinositol glycan anchor biosynthesis class T | 124155 | 244274 | 101443 | 36, 2, 3 |
| 20 | PIGU | phosphatidylinositol glycan anchor biosynthesis class U | 101464 | 101460 | 198646 | 43, 26, 18 |
| 20 | PREX1 | phosphatidylinositol-3,4,5-trisphosphate dependent Rac exchange factor 1 | 124126 | 196562 | 124198 | 25, 36, 26 |
| 20 | PCK1 | phosphoenolpyruvate carboxykinase 1 | 124253 | 124092 | 124256 | 3, 1, 17 |
| 20 | PLCB1 | phospholipase C beta 1 | 182621 | 125827 | 101333 | |
| 20 | PLCB4 | phospholipase C beta 4 | 101333 | 182621 | 125869 | 34, 11, 23 |
| 20 | PLCG1 | phospholipase C gamma 1 | 124181 | 198900 | 174306 | 37, |
| 20 | PLTP | phospholipid transfer protein | 100979 | 064601 | 100982 | 32, 1, 27 |
| 20 | PLAGL2 | PLAG1 like zinc finger 2 | 126003 | 101337 | 101346 | 29, 11, 16 |
| 20 | PABPC1L | poly(A) binding protein cytoplasmic 1 like | 101104 | 166913 | 025772 | 40, 29, 22 |
| 20 | KCNK15 | potassium two pore domain channel subfamily K member 15 | 124249 | 064205 | 101098 | 23, 33, 44 |
| 20 | KCNG1 | potassium voltage-gated channel modifier subfamily G member 1 | 026559 | 124217 | 101096 | |
| 20 | KCNS1 | potassium voltage-gated channel modifier subfamily S member 1 | 124134 | 101109 | 175121 | 8, 35, 44 |
| 20 | KCNB1 | potassium voltage-gated channel subfamily B member 1 | 158445 | 124201 | 124212 | 14, 8, 10 |
| 20 | KCNQ2 | potassium voltage-gated channel subfamily Q member 2 | 075043 | 101204 | 101210 | |
| 20 | PFDN4 | prefoldin subunit 4 | 101132 | 019186 | 280041 | 29, 28, 12 |
| 20 | PRELID3B | PRELI domain containing 3B | 101166 | 124172 | 124203 | 11, 4, 6 |
| 20 | PRPF6 | pre-mRNA processing factor 6 | 101161 | 130590 | 196421 | 28, 21, 13 |
| 20 | PRNP | prion protein | 171867 | 171873 | 171864 | 8, 26, 17 |
| 20 | PRNT | Prion protein (testis specific) | 180259 | 171864 | 101265 | 38, |
| 20 | PRND | prion protein 2 (dublet) | 171864 | 171867 | 180259 | 38, 15, 44 |
| 20 | PDYN | prodynorphin | 101327 | 198053 | 125834 | 8, 38, 17 |
| 20 | PROKR2 | prokineticin receptor 2 | 101292 | 101290 | 125772 | 25, 8, |
| 20 | PCNA | proliferating cell nuclear antigen | 132646 | 089063 | 101290 | |
| 20 | PCSK2 | proprotein convertase subtilisin/kexin type 2 | 125851 | 125879 | 125864 | 28, 8, 40 |
| 20 | PTGIS | prostaglandin I2 synthase | 124212 | 158445 | 158470 | 1, 44, 43 |
| 20 | PMEPA1 | prostate transmembrane protein, androgen induced 1 | 124225 | 124256 | 124237 | 1, 33, 28 |
| 20 | PSMF1 | proteasome inhibitor subunit 1 | 125818 | 101282 | 125895 | 2, 43, 24 |
| 20 | PSMA7 | proteasome subunit alpha 7 | 101182 | 149657 | 184402 | 43, 34, 41 |
| 20 | PROCR | protein C receptor | 101000 | 088298 | 126005 | 43, 18, 15 |
| 20 | POFUT1 | protein O-fucosyltransferase 1 | 101346 | 126003 | 101350 | 27, 33, 31 |
| 20 | PPP1R16B | protein phosphatase 1 regulatory subunit 16B | 101445 | 101442 | 101447 | 22, 41, 21 |
| 20 | PPP1R3D | protein phosphatase 1 regulatory subunit 3D | 132825 | 196227 | 124215 | 26, 15, 22 |
| 20 | PTK6 | protein tyrosine kinase 6 | 101213 | 125534 | 125508 | 9, 13, 24 |
| 20 | PTPN1 | protein tyrosine phosphatase, non-receptor type 1 | 196396 | 172216 | 042062 | 24, 39, 42 |
| 20 | PTPRA | protein tyrosine phosphatase, receptor type A | 132670 | 215305 | 125787 | 31, 27, 5 |
| 20 | PTPRT | protein tyrosine phosphatase, receptor type T | 196090 | 124177 | 124193 | 25, 14, 8 |
| 20 | PCMTD2 | protein-L-isoaspartate (D-aspartate) O-methyltransferase domain containing 2 | 203880 | 196132 | 124209 | 11, 31, 42 |
| 20 | R3HDML | R3H domain containing like | 101074 | 197296 | 101076 | 10, |
| 20 | RAB22A | RAB22A, member RAS oncogene family | 124209 | 124227 | 124164 | 4, 26, 11 |
| 20 | RAD21L1 | RAD21 cohesin complex component like 1 | 244588 | 215595 | 101298 | 38, |
| 20 | RALGAPA2 | Ral GTPase activating protein catalytic alpha subunit 2 | 188559 | 173404 | 088970 | 20, 18, 25 |
| 20 | RALGAPB | Ral GTPase activating protein non-catalytic beta subunit | 170471 | 129988 | 182035 | 22, 23, 1 |
| 20 | RALY | RALY heterogeneous nuclear ribonucleoprotein | 125970 | 101421 | 125977 | 34, 3, 41 |
| 20 | RBCK1 | RANBP2-type and C3HC4-type zinc finger containing 1 | 125826 | 101255 | 125875 | |
| 20 | RIN2 | Ras and Rab interactor 2 | 132669 | 185052 | 173418 | 30, 11, 45 |
| 20 | RASSF2 | Ras association domain family member 2 | 101265 | 180259 | 089057 | 41, 22, 21 |
| 20 | RBBP9 | RB binding protein 9, serine hydrolase | 089050 | 132664 | 101310 | 40, 1, 31 |
| 20 | RBL1 | RB transcriptional corepressor like 1 | 080839 | 101347 | 101353 | 24, 35, 5 |
| 20 | RBBP8NL | RBBP8 N-terminal like | 130701 | 149679 | 130700 | 13, 29, 24 |
| 20 | ROMO1 | reactive oxygen species modulator 1 | 125995 | 244005 | 131051 | 25, 43, 2 |
| 20 | RBPJL | recombination signal binding protein for immunoglobulin kappa J region like | 124232 | 124159 | 124145 | 28, 4, |
| 20 | RIMS4 | regulating synaptic membrane exocytosis 4 | 101098 | 124249 | 166913 | 8, 38, 9 |
| 20 | RPRD1B | regulation of nuclear pre-mRNA domain containing 1B | 101413 | 101407 | 198959 | 42, 24, 44 |
| 20 | RGS19 | regulator of G-protein signaling 19 | 171700 | 171703 | 125510 | 22, 9, 3 |
| 20 | RTEL1 | regulator of telomere elongation helicase 1 | 258366 | 197457 | 026036 | 21, 16, 37 |
| 20 | RTFDC1 | replication termination factor 2 domain containing 1 | 022277 | 087589 | 124091 | 41, 34, 6 |
| 20 | ARHGAP40 | Rho GTPase activating protein 40 | 124143 | 182035 | 101438 | |
| 20 | RAE1 | ribonucleic acid export 1 | 101146 | 054796 | 256222 | 38, 30, 18 |
| 20 | RPN2 | ribophorin II | 118705 | 101353 | 118702 | 13, 40, 9 |
| 20 | RPS21 | ribosomal protein S21 | 171858 | 130702 | 149679 | 33, 41, 7 |
| 20 | RRBP1 | ribosome binding protein 1 | 125844 | 125868 | 125888 | 13, 29, 28 |
| 20 | RNF114 | ring finger protein 114 | 124226 | 158480 | 124216 | 9, 18, 3 |
| 20 | RNF24 | ring finger protein 24 | 101236 | 125779 | 088826 | 1, 34, 13 |
| 20 | RBM12 | RNA binding motif protein 12 | 244462 | 272897 | 244005 | 42, 45, 39 |
| 20 | RBM38 | RNA binding motif protein 38 | 132819 | 256222 | 124092 | 21, 41, 33 |
| 20 | RBM39 | RNA binding motif protein 39 | 131051 | 125995 | 025293 | 7, 1, 31 |
| 20 | POLR3F | RNA polymerase III subunit F | 132664 | 089091 | 089050 | 26, 31, 35 |
| 20 | REM1 | RRAD and GEM like GTPase 1 | 088320 | 180383 | 101294 | 15, 32, 6 |
| 20 | RSPO4 | R-spondin 4 | 101282 | 101280 | 125818 | 14, 10, 38 |
| 20 | RTEL1- | RTEL1-TNFRSF6B readthrough (NMD candidate) | 026036 | 258366 | 243509 | |
| | TNFRSF6B | | | | | |
| 20 | SUN5 | Sad1 and UNC84 domain containing 5 | 167098 | 215529 | 078898 | 38, 8, |
| 20 | SAMHD1 | SAM and HD domain containing deoxynucleoside triphosphate | 101347 | 101342 | 080839 | 42, 13, 36 |
| | | triphosphohydrolase 1 | | | | |
| 20 | SCAND1 | SCAN domain containing 1 | 171222 | 025293 | 149646 | 22, 35, 6 |
| 20 | SCP2D1 | SCP2 sterol binding domain containing 1 | 132631 | 125821 | 185052 | 38, |
| 20 | SCRT2 | scratch family transcriptional repressor 2 | 215397 | 270299 | 101276 | 14, 28, 8 |
| 20 | SEC23B | Sec23 homolog B, coat complex II component | 101310 | 089050 | 232388 | 24, 42, 31 |
| 20 | SLPI | secretory leukocyte peptidase inhibitor | 124107 | 124157 | 124159 | 30, 18, 27 |
| 20 | SEL1L2 | SEL1L2 ERAD E3 ligase adaptor subunit | 101251 | 101247 | 172264 | 10, 42, 38 |
| 20 | SEMG1 | semenogelin I | 124233 | 124102 | 124157 | 33, 25, 23 |
| 20 | SEMG2 | semenogelin II | 124157 | 124233 | 124107 | 33, 25, 42 |
| 20 | SRSF6 | serine and arginine rich splicing factor 6 | 124193 | 196090 | 185513 | 11, 31, 13 |
| 20 | SERINC3 | serine incorporator 3 | 132824 | 124120 | 168734 | 38, 30, 45 |
| 20 | SPTLC3 | serine palmitoyltransferase long chain base subunit 3 | 172296 | 132640 | 101230 | 11, 32, 29 |
| 20 | SPINT3 | serine peptidase inhibitor, Kunitz type 3 | 101446 | 101443 | 243543 | 38, |
| 20 | SPINT4 | serine peptidase inhibitor, Kunitz type 4 | 149651 | 168634 | 124116 | |
| 20 | STK35 | serine/threonine kinase 35 | 125834 | 101327 | 125780 | 14, 39, 18 |
| 20 | STK4 | serine/threonine kinase 4 | 101109 | 025772 | 124134 | 22, 30, 5 |
| 20 | SGK2 | SGK2, serine/threonine kinase 2 | 101049 | 277611 | 101052 | |
| 20 | SIGLEC1 | sialic acid binding Ig like lectin 1 | 088827 | 149451 | 132622 | 1, 27, 6 |
| 20 | SIRPA | signal regulatory protein alpha | 198053 | 089012 | 101327 | 35, 1, 8 |
| 20 | SIRPB1 | signal regulatory protein beta 1 | 101307 | 260861 | 089012 | 5, 36, 7 |
| 20 | SIRPB2 | signal regulatory protein beta 2 | 196209 | 088833 | 125900 | 18, 5, 7 |
| 20 | SIRPD | signal regulatory protein delta | 125900 | 196209 | 260861 | 38, 36, 5 |
| 20 | SIRPG | signal regulatory protein gamma | 089012 | 101307 | 198053 | 39, 30, 38 |
| 20 | SLC2A4RG | SLC2A4 regulator | 125520 | 273047 | 130584 | 34, 6, 17 |
| 20 | SLX4IP | SLX4 interacting protein | 149346 | 125863 | 101384 | 33, 39, 28 |
| 20 | SNRPB2 | small nuclear ribonucleoprotein polypeptide B2 | 125870 | 280240 | 125879 | 34, 11, 4 |
| 20 | SNRPB | small nuclear ribonucleoprotein polypeptides B and B1 | 125835 | 166948 | 256566 | 41, 35, 34 |
| 20 | SNAI1 | snail family transcriptional repressor 1 | 124216 | 124226 | 124208 | 3, 45, 6 |
| 20 | NKAIN4 | Sodium/potassium transporting ATPase interacting 4 | 101198 | 101197 | 101199 | 17, 26, 8 |
| 20 | SLC12A5 | solute carrier family 12 member 5 | 124140 | 100985 | 124160 | 14, 8, 31 |
| 20 | SLC13A3 | solute carrier family 13 member 3 | 158296 | 149635 | 172315 | 17, 32, 11 |
| 20 | SLC17A9 | solute carrier family 17 member 9 | 101194 | 101193 | 125533 | 22, 3, 42 |
| 20 | SLC2A10 | solute carrier family 2 member 10 | 197496 | 172315 | 064655 | 29, 23, 1 |
| 20 | SLC23A2 | solute carrier family 23 member 2 | 089057 | 101265 | 089063 | 29, 2, 14 |
| 20 | SLC24A3 | solute carrier family 24 member 3 | 185052 | 132631 | 132669 | 30, 11, 25 |
| 20 | SLC32A1 | solute carrier family 32 member 1 | 101438 | 124143 | 101442 | 8, 14, 17 |
| 20 | SLC35C2 | solute carrier family 35 member C2 | 080189 | 149654 | 062598 | 36, 13, 9 |
| 20 | SLC4A11 | solute carrier family 4 member 11 | 088836 | 125877 | 088854 | 24, 3, 27 |
| 20 | SLC52A3 | solute carrier family 52 member 3 | 101276 | 215397 | 125898 | 38, 34, 33 |
| 20 | SLC9A8 | solute carrier family 9 member A8 | 197818 | 158470 | 158480 | 7, 22, 3 |
| 20 | SLCO4A1 | solute carrier organic anion transporter family member 4A1 | 101187 | 130700 | 101188 | 14, 16, 3 |
| 20 | SSTR4 | somatostatin receptor 4 | 132671 | 125798 | 178726 | |
| 20 | SNX5 | sorting nexin 5 | 089006 | 125888 | 125850 | 29, 32, 18 |
| 20 | SNX21 | sorting nexin family member 21 | 124104 | 101470 | 101473 | 26, 14, 31 |
| 20 | SALL4 | spalt like transcription factor 4 | 101115 | 054793 | 020256 | 12, 2, 38 |
| 20 | SPAG4 | sperm associated antigen 4 | 061656 | 125991 | 214078 | 11, 7, 4 |
| 20 | SPEF1 | sperm flagellar 1 | 101222 | 101220 | 125817 | 38, 24, 3 |
| 20 | SPATA2 | spermatogenesis associated 2 | 158480 | 197818 | 124226 | 38, 34, 24 |
| 20 | SPATA25 | spermatogenesis associated 25 | 149634 | 168612 | 124257 | 37, 38, 8 |
| 20 | SMOX | spermine oxidase | 088826 | 101236 | 171873 | 9, 33, 7 |
| 20 | SPO11 | SPO11, initiator of meiotic double stranded breaks | 054796 | 101144 | 101146 | 10, 8, |
| 20 | SLA2 | Src like adaptor 2 | 101082 | 101084 | 101079 | 5, 7, 39 |
| 20 | SRC | SRC proto-oncogene, non-receptor tyrosine kinase | 197122 | 101363 | 166619 | 43, 4, 20 |
| 20 | SRMS | src-related kinase lacking C-terminal regulatory tyrosine and N-terminal | 125508 | 101213 | 125531 | 23, 37, 17 |
| | | myristylation sites | | | | |
| 20 | SOX12 | SRY-box 12 | 177732 | 247315 | 125841 | 26, 14, 12 |
| 20 | SOX18 | SRY-box 18 | 203883 | 196421 | 171703 | 32, 27, 28 |
| 20 | SS18L1 | SS18L1, nBAF chromatin remodeling complex subunit | 184402 | 101182 | 101181 | 19, 1, 26 |
| 20 | STMN3 | stathmin 3 | 197457 | 101216 | 258366 | 8, 26, 39 |
| 20 | STAU1 | staufen double-stranded RNA binding protein 1 | 124214 | 124207 | 124228 | 39, 42, 3 |
| 20 | SAMD10 | sterile alpha motif domain containing 10 | 130590 | 196700 | 101161 | 9, 37, 40 |
| 20 | STX16-NPEPL1 | STX16-NPEPL1 readthrough (NMD candidate) | 254995 | 124222 | 215440 | |
| 20 | SULF2 | sulfatase 2 | 196562 | 124151 | 124126 | 8, |
| 20 | SRXN1 | sulfiredoxin 1 | 271303 | 125878 | 270299 | 20, 40, 3 |
| 20 | SOGA1 | suppressor of glucose, autophagy associated 1 | 149639 | 149636 | 101342 | 26, 23, 2 |
| 20 | SYNDIG1 | synapse differentiation inducing 1 | 101463 | 149435 | 077984 | 43, 8, 27 |
| 20 | SYCP2 | synaptonemal complex protein 2 | 196074 | 087495 | 196227 | 37, 23, 38 |
| 20 | SNAP25 | synaptosome associated protein 25 | 132639 | 132623 | 125863 | 8, 17, 26 |
| 20 | SDC4 | syndecan 4 | 124145 | 124232 | 204070 | 13, 4, 40 |
| 20 | SDCBP2 | syndecan binding protein 2 | 125775 | 101298 | 274322 | 13, 16, 26 |
| 20 | SNPH | syntaphilin | 101298 | 244588 | 125775 | 29, 13, 18 |
| 20 | STX16 | syntaxin 16 | 124222 | 198768 | 254995 | 31, 2, 12 |
| 20 | SNTA1 | syntrophin alpha 1 | 101400 | 101391 | 078699 | 21, 18, 15 |
| 20 | SYS1 | SYS1, golgi trafficking protein | 204070 | 124145 | 254806 | 41, 1, 13 |
| 20 | SYS1-DBNDD2 | SYS1-DBNDD2 readthrough (NMD candidate) | 254806 | 204070 | 124251 | |
| 20 | TASP1 | taspase 1 | 089123 | 101230 | 089048 | 18, 31, 13 |
| 20 | TAF4 | TATA-box binding protein associated factor 4 | 130699 | 179242 | 149657 | 29, 11, 31 |
| 20 | TLDC2 | TBC/LysM-associated domain containing 2 | 101342 | 149639 | 101347 | 37, 4, 2 |
| 20 | TBC1D20 | TBC1 domain family member 20 | 125875 | 125826 | 101266 | 43, 34, 35 |
| 20 | TSHZ2 | teashirt zinc finger homeobox 2 | 182463 | 020256 | 171940 | 24, 1, 31 |
| 20 | TTI1 | TELO2 interacting protein 1 | 101407 | 132821 | 101413 | 9, 42, 21 |
| 20 | TGIF2 | TGFB induced factor homeobox 2 | 118707 | 101335 | 259399 | 34, 13, 25 |
| 20 | TGIF2-C20orf24 | TGIF2-C20orf24 readthrough | 259399 | 118707 | 101084 | |
| 20 | TMX4 | thioredoxin related transmembrane protein 4 | 125827 | 101323 | 182621 | 31, 38, 29 |
| 20 | THBD | thrombomodulin | 178726 | 132671 | 125810 | 36, 24, 45 |
| 20 | TMEM189- | TMEM189-UBE2V1 readthrough | 124208 | 124216 | 240849 | 18, 5, 13 |
| | UBE2V1 | | | | | |
| 20 | TNFRSF6B | TNF receptor superfamily member 6b | 243509 | 026036 | 101246 | 3, 37, 26 |
| 20 | TOP1 | topoisomerase (DNA) I | 198900 | 204103 | 124181 | 24, 26, 13 |
| 20 | TOX2 | TOX high mobility group box family member 2 | 124191 | 124196 | 149596 | 39, 34, 28 |
| 20 | TP53RK | TP53 regulating kinase | 172315 | 158296 | 197496 | 26, 19, 7 |
| 20 | TP53TG5 | TP53 target 5 | 124251 | 254806 | 244274 | 6, 38, 8 |
| 20 | TPX2 | TPX2, microtubule nucleation factor | 088325 | 171552 | 101306 | 3, 39, 24 |
| 20 | TCEA2 | transcription elongation factor A2 | 171703 | 203883 | 171700 | 11, 27, 38 |
| 20 | TCF15 | transcription factor 15 (basic helix-loop-helix) | 125878 | 101266 | 271303 | 20, 15, 14 |
| 20 | TFAP2C | transcription factor AP-2 gamma | 087510 | 213714 | 101144 | 34, 27, 18 |
| 20 | TCFL5 | transcription factor like 5 | 101190 | 092758 | 101191 | 35, 26, 38 |
| 20 | TGM2 | transglutaminase 2 | 198959 | 101413 | 149633 | 43, 32, 44 |
| 20 | TGM3 | transglutaminase 3 | 125780 | 125834 | 166948 | 24, 13, 30 |
| 20 | TGM6 | transglutaminase 6 | 166948 | 125780 | 125835 | 20, |
| 20 | TRPC4AP | transient receptor potential cation channel subfamily C member 4 associated | 100991 | 078814 | 088298 | 21, 39, 43 |
| | | protein | | | | |
| 20 | TOMM34 | translocase of outer mitochondrial membrane 34 | 025772 | 101104 | 101109 | 3, 38, 2 |
| 20 | TM9SF4 | transmembrane 9 superfamily member 4 | 101337 | 101336 | 126003 | 24, 42, 13 |
| 20 | TMC2 | transmembrane channel like 2 | 149488 | 088876 | 101361 | 36, 8, 44 |
| 20 | TMEM189 | transmembrane protein 189 | 240849 | 124208 | 172216 | |
| 20 | TMEM230 | transmembrane protein 230 | 089063 | 089057 | 132646 | 43, 31, 28 |
| 20 | TMEM239 | transmembrane protein 239 | 198326 | 258713 | 132635 | 38, 8, |
| 20 | TMEM74B | transmembrane protein 74B | 125895 | 125818 | 215595 | 34, 29, 10 |
| 20 | TRIB3 | tribbles pseudokinase 3 | 101255 | 125841 | 125826 | 9, 41, 13 |
| 20 | TRMT6 | tRNA methyltransferase 6 | 089195 | 089199 | 125885 | 9, 13, 18 |
| 20 | TNNC2 | troponin C2, fast skeletal type | 101470 | 175063 | 124104 | 25, 15, 18 |
| 20 | TUBB1 | tubulin beta 1 class VI | 101162 | 101160 | 124172 | 11, 7, 36 |
| 20 | TTLL9 | tubulin tyrosine ligase like 9 | 131044 | 149599 | 088356 | 31, 38, 10 |
| 20 | TPD52L2 | tumor protein D52 like 2 | 101150 | 183260 | 101152 | 45, 1, 35 |
| 20 | TP53INP2 | tumor protein p53 inducible nuclear protein 2 | 078804 | 198646 | 131067 | 26, 11, 14 |
| 20 | YWHAB | tyrosine 3-monooxygenase/tryptophan 5-monooxygenase activation protein beta | 166913 | 101098 | 101104 | 11, 4, 40 |
| 20 | UQCC1 | ubiquinol-cytochrome c reductase complex assembly factor 1 | 101019 | 125998 | 204183 | |
| 20 | UBE2C | ubiquitin conjugating enzyme E2 C | 175063 | 101457 | 101470 | 41, 3, 11 |
| 20 | UBE2V1 | ubiquitin conjugating enzyme E2 V1 | 244687 | 124216 | 240849 | |
| 20 | UBOX5 | U-box domain containing 5 | 185019 | 101200 | 215251 | 29, 25, 6 |
| 20 | UCKL1 | uridine-cytidine kinase 1 like 1 | 198276 | 101152 | 196700 | 14, 13, 37 |
| 20 | VAPB | VAMP associated protein B and C | 124164 | 124209 | 198768 | 34, 2, 17 |
| 20 | VSX1 | visual system homeobox 1 | 100987 | 154930 | 197586 | 14, 10, 8 |
| 20 | VPS16 | VPS16, CORVET/HOPS core subunit | 215305 | 132635 | 132670 | |
| 20 | VSTM2L | V-set and transmembrane domain containing 2 like | 132821 | 132792 | 101407 | 28, 8, 20 |
| 20 | WFDC10A | WAP four-disulfide core domain 10A | 180305 | 180205 | 180083 | 10, 44, 32 |
| 20 | WFDC10B | WAP four-disulfide core domain 10B | 182931 | 180083 | 168634 | 38, 8, |
| 20 | WFDC11 | WAP four-disulfide core domain 11 | 180083 | 180305 | 182931 | 38, |
| 20 | WFDC12 | WAP four-disulfide core domain 12 | 168703 | 175121 | 124102 | 35, |
| 20 | WFDC13 | WAP four-disulfide core domain 13 | 168634 | 182931 | 149651 | |
| 20 | WFDC2 | WAP four-disulfide core domain 2 | 101443 | 124155 | 101446 | 27, 40, 15 |
| 20 | WFDC3 | WAP four-disulfide core domain 3 | 124116 | 149651 | 101457 | 14, 38, 19 |
| 20 | WFDC5 | WAP four-disulfide core domain 5 | 175121 | 124134 | 168703 | 35, 30, 24 |
| 20 | WFDC6 | WAP four-disulfide core domain 6 | 243543 | 101446 | 249139 | 38, 10, |
| 20 | WFDC8 | WAP four-disulfide core domain 8 | 158901 | 101448 | 180205 | 38, |
| 20 | WFDC9 | WAP four-disulfide core domain 9 | 180205 | 158901 | 180305 | |
| 20 | WISP2 | WNT1 inducible signaling pathway protein 2 | 064205 | 196839 | 124249 | 23, 9, 27 |
| 20 | XKR7 | XK related 7 | 260903 | 088356 | 101331 | 20, 14, |
| 20 | YTHDF1 | YTH N6-methyladenosine RNA binding protein 1 | 149658 | 125533 | 101197 | 37, 21, 3 |
| 20 | ZBP1 | Z-DNA binding protein 1 | 124256 | 124253 | 124225 | 5, 13, 9 |
| 20 | ZFP64 | ZFP64 zinc finger protein | 020256 | 101115 | 182463 | 29, 21, 24 |
| 20 | ZBTB46 | zinc finger and BTB domain containing 46 | 130584 | 125520 | 183260 | 1, 27, 33 |
| 20 | ZGPAT | zinc finger COCH-type and G-patch domain containing | 197114 | 101246 | 273154 | 36, 39, 13 |
| 20 | ZCCHC3 | zinc finger CCHC-type containing 3 | 247315 | 196476 | 177732 | 15, 26, 31 |
| 20 | ZMYND8 | zinc finger MYND-type containing 8 | 101040 | 064655 | 124151 | |
| 20 | ZNFX1 | zinc finger NFX1-type containing 1 | 124201 | 124228 | 158445 | |
| 20 | ZNF133 | zinc finger protein 133 | 125846 | 149474 | 089091 | 15, 43, 26 |
| 20 | ZNF217 | zinc finger protein 217 | 171940 | 182463 | 064787 | 3, 23, 29 |
| 20 | ZNF334 | zinc finger protein 334 | 198185 | 062598 | 149635 | 26, 31, 33 |
| 20 | ZNF335 | zinc finger protein 335 | 198026 | 100982 | 100985 | 29, 36, 22 |
| 20 | ZNF337 | zinc finger protein 337 | 130684 | 170191 | 175170 | 24, 42, 1 |
| 20 | ZNF341 | zinc finger protein 341 | 131061 | 101417 | 101421 | 4, 21, 33 |
| 20 | ZNF343 | zinc finger protein 343 | 088876 | 256566 | 149488 | 1, 4, 41 |
| 20 | ZNF512B | zinc finger protein 512B | 196700 | 198276 | 130590 | 40, 28, 18 |
| 20 | ZNF831 | zinc finger protein 831 | 124203 | 101166 | 124205 | 36, |
| 20 | ZSWIM1 | zinc finger SWIM-type containing 1 | 168612 | 132801 | 149634 | 7, 45, 13 |
| 20 | ZSWIM3 | zinc finger SWIM-type containing 3 | 132801 | 101473 | 168612 | 9, 33, 39 |
| 20 | ZHX3 | zinc fingers and homeoboxes 3 | 174306 | 124181 | 132793 | |
| 21 | AGPAT3 | 1-acylglycerol-3-phosphate O-acyltransferase 3 | 160216 | 160214 | 160218 | |
| 21 | ADAMTS1 | ADAM metallopeptidase with thrombospondin type 1 motif 1 | 154734 | 166265 | 154736 | 26, 33, 44 |
| 21 | ADAMTS5 | ADAM metallopeptidase with thrombospondin type 1 motif 5 | 154736 | 154734 | 156239 | 15, 1, 11 |
| 21 | ADARB1 | adenosine deaminase, RNA specific B1 | 197381 | 160256 | 186866 | |
| 21 | APP | amyloid beta precursor protein | 142192 | 154727 | 166265 | 40, 11, 18 |
| 21 | ABCG1 | ATP binding cassette subfamily G member 1 | 160179 | 177398 | 160180 | 26, 39, 14 |
| 21 | ATP5O | ATP synthase, H+ transporting, mitochondrial F1 complex, O subunit | 241837 | 205726 | 243927 | 43, 29, 41 |
| 21 | ATP5J | ATP synthase, H+ transporting, mitochondrial Fo complex subunit F6 | 154723 | 154721 | 154727 | 29, 15, 2 |
| 21 | AIRE | autoimmune regulator | 160224 | 142182 | 141959 | 39, 17, |
| 21 | B3GALT5 | beta-1,3-galactosyltransferase 5 | 183778 | 185437 | 183067 | 42, 16, 23 |
| 21 | BACE2 | beta-site APP-deaving enzyme 2 | 182240 | 171587 | 183844 | 3, 9, 35 |
| 21 | BRWD1 | bromodomain and WD repeat domain containing 1 | 185658 | 183527 | 205581 | 23, 22, 39 |
| 21 | BACH1 | BTB domain and CNC homolog 1 | 156273 | 156265 | 171189 | 2, 13, 42 |
| 21 | BTG3 | BTG anti-proliferation factor 3 | 154640 | 154639 | 154642 | 18, 6, 32 |
| 21 | C2CD2 | C2 calcium dependent domain containing 2 | 157617 | 141956 | 173276 | 2, 29, 1 |
| 21 | CBR1 | carbonyl reductase 1 | 159228 | 185917 | 159231 | 13, 8, 3 |
| 21 | CBR3 | carbonyl reductase 3 | 159231 | 159228 | 142197 | 24, 34, 1 |
| 21 | CCT8 | chaperonin containing TCP1 subunit 8 | 156261 | 156256 | 156265 | 13, 43, 4 |
| 21 | CLIC6 | chloride intracellular channel 6 | 159212 | 159200 | 159216 | 20, 18, 14 |
| 21 | CHODL | chondrolectin | 154645 | 154642 | 154646 | 36, 38, 10 |
| 21 | CHAF1B | chromatin assembly factor 1 subunit B | 159259 | 159256 | 159261 | 9, 26, 13 |
| 21 | C21orf140 | chromosome 21 open reading frame 140 | 222018 | 205670 | 243627 | |
| 21 | C21orf2 | chromosome 21 open reading frame 2 | 160226 | 141959 | 142185 | 26, 27, 22 |
| 21 | C21orf33 | chromosome 21 open reading frame 33 | 160221 | 241945 | 160223 | 21, 9, 34 |
| 21 | C21orf58 | chromosome 21 open reading frame 58 | 160298 | 182362 | 160299 | 39, 9, 26 |
| 21 | C21orf59 | chromosome 21 open reading frame 59 | 159079 | 265590 | 159082 | 34, 28, 41 |
| 21 | C21orf62 | chromosome 21 open reading frame 62 | 205929 | 159086 | 205927 | 28, 37, 9 |
| 21 | C21orf91 | chromosome 21 open reading frame 91 | 154642 | 154640 | 154645 | 22, 4, 11 |
| 21 | CLDN14 | daudin 14 | 159261 | 159259 | 159263 | |
| 21 | CLDN17 | daudin 17 | 156282 | 171189 | 156284 | |
| 21 | CLDN8 | daudin 8 | 156284 | 156282 | 188694 | 17, 33, 13 |
| 21 | COL6A1 | collagen type VI alpha 1 chain | 142156 | 183570 | 142173 | 6, 26, 15 |
| 21 | COL6A2 | collagen type VI alpha 2 chain | 142173 | 142156 | 160282 | 6, 12, 26 |
| 21 | COL18A1 | collagen type XVIII alpha 1 chain | 182871 | 186866 | 173638 | 26, 27, 12 |
| 21 | CXADR | coxsackie virus and adenovirus receptor | 154639 | 155313 | 154640 | 11, 18, 4 |
| 21 | CRYAA | crystallin alpha A | 160202 | 160201 | 142178 | 14, 17, 9 |
| 21 | CRYZL1 | crystallin zeta like 1 | 205758 | 249209 | 205726 | 30, 31, 15 |
| 21 | CBS | cystathionine-beta-synthase | 160200 | 160199 | 160201 | 21, 26, 9 |
| 21 | CBSL | cystathionine-beta-synthase like | 274276 | 275993 | 275895 | 21, 26, 9 |
| 21 | CSTB | cystatin B | 160213 | 160209 | 160214 | 30, 29, 18 |
| 21 | CYYR1 | cysteine and tyrosine rich 1 | 166265 | 142192 | 154734 | 1, 43, 2 |
| 21 | DIP2A | disco interacting protein 2 homolog A | 160305 | 160299 | 160307 | 37, 40, 1 |
| 21 | DNMT3L | DNA methyltransferase 3 like | 142182 | 160223 | 160224 | 32, 38, |
| 21 | DNAJC28 | DnaJ heat shock protein family (Hsp40) member C28 | 177692 | 142188 | 159131 | 30, 42, 6 |
| 21 | DOPEY2 | dopey family member 2 | 142197 | 159231 | 159256 | 13, 5, 33 |
| 21 | DSCR4 | Down syndrome critical region 4 | 184029 | 157542 | 198054 | |
| 21 | DSCR8 | Down syndrome critical region 8 | 198054 | 184029 | 157551 | 10, 34, 38 |
| 21 | DONSON | downstream neighbor of SON | 159147 | 159140 | 249209 | 11, 9, 4 |
| 21 | DSCAM | DS cell adhesion molecule | 171587 | 183036 | 182240 | 23, 8, 25 |
| 21 | DSCR3 | DSCR3 arrestin fold containing | 157538 | 182670 | 157540 | 39, 34, 37 |
| 21 | DYRK1A | dual specificity tyrosine phosphorylation regulated kinase 1A | 157540 | 157538 | 157542 | 11, 24, 31 |
| 21 | ERG | ERG, ETS transcription factor | 157554 | 157551 | 157557 | 45, 1, 36 |
| 21 | ETS2 | ETS proto-oncogene 2, transcription factor | 157557 | 157554 | 183527 | 1, 42, 18 |
| 21 | EVA1C | eva-1 homolog C | 166979 | 142207 | 242220 | 9, 32, 36 |
| 21 | FAM207A | family with sequence similarity 207 member A | 160256 | 160255 | 197381 | 21, 35, 25 |
| 21 | FAM3B | family with sequence similarity 3 member B | 183844 | 182240 | 183486 | 37, 31, 27 |
| 21 | FTCD | formimidoyltransferase cyclodeaminase | 160282 | 142173 | 160284 | 17, 19, 38 |
| 21 | GABPA | GA binding protein transcription factor alpha subunit | 154727 | 154723 | 142192 | 42, 32, 13 |
| 21 | GRIK1 | glutamate ionotropic receptor kainate type subunit 1 | 171189 | 156273 | 156282 | 8, 22, 15 |
| 21 | H2BFS | H2B histone family member S | 234289 | 160207 | 160208 | |
| 21 | HSPA13 | heat shock protein family A (Hsp70) member 13 | 155304 | 185272 | 155307 | 42, 4, 8 |
| 21 | HSF2BP | heat shock transcription factor 2 binding protein | 160207 | 142178 | 234289 | 13, 9, 38 |
| 21 | HMGN1 | high mobility group nucleosome binding domain 1 | 205581 | 185658 | 182093 | 18, 12, 29 |
| 21 | HLCS | holocarboxylase synthetase | 159267 | 159263 | 183145 | 26, 29, 18 |
| 21 | HUNK | hormonally up-regulated Neu-associated kinase | 142149 | 156304 | 159055 | 11, 27, 12 |
| 21 | IGHV1OR21-1 | Immunoglobulin heavy variable 1/OR21-1 (non-functional) | 277282 | 274391 | 280108 | |
| 21 | IGSF5 | immunoglobulin superfamily member 5 | 183067 | 183778 | 183036 | 32, 17, 38 |
| 21 | ICOSLG | inducible T-cell costimulator ligand | 160223 | 160221 | 142182 | 8, 2, 41 |
| 21 | ITGB2 | integrin subunit beta 2 | 160255 | 183255 | 160256 | 39, 36, 5 |
| 21 | IFNAR1 | interferon alpha and beta receptor subunit 1 | 142166 | 243646 | 159128 | 4, 45, 42 |
| 21 | IFNAR2 | interferon alpha and beta receptor subunit 2 | 159110 | 184221 | 249624 | 1, 15, 36 |
| 21 | IFNGR2 | interferon gamma receptor 2 | 159128 | 142166 | 142188 | |
| 21 | IL10RB | interleukin 10 receptor subunit beta | 243646 | 249624 | 142166 | 13, 37, 9 |
| 21 | ITSN1 | intersectin 1 | 205726 | 205758 | 241837 | 26, 11, 1 |
| 21 | JAM2 | junctional adhesion molecule 2 | 154721 | 154719 | 154723 | 11, 42, 32 |
| 21 | KRTAP10-1 | keratin associated protein 10-1 | 215455 | 175894 | 205445 | |
| 21 | KRTAP10-10 | keratin associated protein 10-10 | 221859 | 221837 | 243489 | |
| 21 | KRTAP10-11 | keratin associated protein 10-11 | 243489 | 221859 | 205439 | 15, |
| 21 | KRTAP10-12 | keratin associated protein 10-12 | 189169 | 187175 | 184787 | 27, 14, |
| 21 | KRTAP10-2 | keratin associated protein 10-2 | 205445 | 215455 | 212935 | |
| 21 | KRTAP10-3 | keratin associated protein 10-3 | 212935 | 205445 | 215454 | |
| 21 | KRTAP10-4 | keratin associated protein 10-4 | 215454 | 212935 | 241123 | |
| 21 | KRTAP10-5 | keratin associated protein 10-5 | 241123 | 215454 | 188155 | |
| 21 | KRTAP10-6 | keratin associated protein 10-6 | 188155 | 241123 | 272804 | |
| 21 | KRTAP10-7 | keratin associated protein 10-7 | 272804 | 188155 | 187766 | |
| 21 | KRTAP10-8 | keratin associated protein 10-8 | 187766 | 272804 | 221837 | |
| 21 | KRTAP10-9 | keratin associated protein 10-9 | 221837 | 187766 | 221859 | |
| 21 | KRTAP11-1 | keratin associated protein 11-1 | 182591 | 274749 | 206102 | |
| 21 | KRTAP12-1 | keratin associated protein 12-1 | 187175 | 221864 | 189169 | |
| 21 | KRTAP12-2 | keratin associated protein 12-2 | 221864 | 212933 | 187175 | |
| 21 | KRTAP12-3 | keratin associated protein 12-3 | 205439 | 243489 | 212933 | |
| 21 | KRTAP12-4 | keratin associated protein 12-4 | 212933 | 205439 | 221864 | |
| 21 | KRTAP13-1 | keratin associated protein 13-1 | 198390 | 182816 | 240432 | |
| 21 | KRTAP13-2 | keratin associated protein 13-2 | 182816 | 186980 | 198390 | 15, |
| 21 | KRTAP13-3 | keratin associated protein 13-3 | 240432 | 198390 | 186971 | |
| 21 | KRTAP13-4 | keratin associated protein 13-4 | 186971 | 240432 | 186970 | |
| 21 | KRTAP15-1 | keratin associated protein 15-1 | 186970 | 186971 | 184351 | |
| 21 | KRTAP19-1 | keratin associated protein 19-1 | 184351 | 186970 | 186965 | 15, |
| 21 | KRTAP19-2 | keratin associated protein 19-2 | 186965 | 184351 | 244025 | |
| 21 | KRTAP19-3 | keratin associated protein 19-3 | 244025 | 186965 | 186967 | 15, |
| 21 | KRTAP19-4 | keratin associated protein 19-4 | 186967 | 244025 | 186977 | |
| 21 | KRTAP19-5 | keratin associated protein 19-5 | 186977 | 186967 | 186925 | |
| 21 | KRTAP19-6 | keratin associated protein 19-6 | 186925 | 186977 | 244362 | |
| 21 | KRTAP19-7 | keratin associated protein 19-7 | 244362 | 186925 | 206106 | |
| 21 | KRTAP19-8 | keratin associated protein 19-8 | 206102 | 182591 | 156299 | 38, |
| 21 | KRTAP20-1 | keratin associated protein 20-1 | 244624 | 184724 | 206105 | |
| 21 | KRTAP20-2 | keratin associated protein 20-2 | 184032 | 206105 | 206104 | |
| 21 | KRTAP20-3 | keratin associated protein 20-3 | 206104 | 184032 | 231068 | |
| 21 | KRTAP20-4 | Keratin associated protein 20-4 | 206105 | 244624 | 184032 | |
| 21 | KRTAP21-1 | keratin associated protein 21-1 | 187005 | 187026 | 183640 | |
| 21 | KRTAP21-2 | keratin associated protein 21-2 | 187026 | 231068 | 187005 | |
| 21 | KRTAP21-3 | keratin associated protein 21-3 | 231068 | 206104 | 187026 | |
| 21 | KRTAP22-1 | keratin associated protein 22-1 | 186924 | 186930 | 184724 | |
| 21 | KRTAP22-2 | keratin associated protein 22-2 | 206106 | 244362 | 212938 | |
| 21 | KRTAP23-1 | keratin associated protein 23-1 | 186980 | 206107 | 182816 | |
| 21 | KRTAP24-1 | keratin associated protein 24-1 | 188694 | 156284 | 232263 | |
| 21 | KRTAP25-1 | keratin associated protein 25-1 | 232263 | 188694 | 197683 | |
| 21 | KRTAP26-1 | keratin associated protein 26-1 | 197683 | 232263 | 206107 | 32, |
| 21 | KRTAP27-1 | keratin associated protein 27-1 | 206107 | 197683 | 186980 | |
| 21 | KRTAP6-1 | keratin associated protein 6-1 | 184724 | 186924 | 244624 | |
| 21 | KRTAP6-2 | keratin associated protein 6-2 | 186930 | 212938 | 186924 | |
| 21 | KRTAP6-3 | keratin associated protein 6-3 | 212938 | 206106 | 186930 | |
| 21 | KRTAP7-1 | keratin associated protein 7-1 (gene/pseudogene) | 274749 | 183640 | 182591 | |
| 21 | KRTAP8-1 | keratin associated protein 8-1 | 183640 | 187005 | 274749 | |
| 21 | LSS | lanosterol synthase | 160285 | 160284 | 160294 | 43, 8, 21 |
| 21 | LCA5L | LCA5L, lebercilin like | 157578 | 182093 | 185437 | 30, 38, |
| 21 | LRRC3 | leucine rich repeat containing 3 | 160233 | 142185 | 175894 | 9, 44, |
| 21 | LIPI | lipase I | 188992 | 166351 | 185272 | 6, 10, 38 |
| 21 | LTN1 | listerin E3 ubiquitin protein ligase 1 | 198862 | 156239 | 156253 | 30, 7, 29 |
| 21 | MAP3K7CL | MAP3K7 C-terminal like | 156265 | 156261 | 156273 | 22, 45, 25 |
| 21 | MRAP | melanocortin 2 receptor accessory protein | 170262 | 159055 | 142207 | 3, 2, |
| 21 | MCM3AP | minichromosome maintenance complex component 3 associated protein | 160294 | 160285 | 182362 | 34, 25, 36 |
| 21 | MIS18A | MIS18 kinetochore protein A | 159055 | 142149 | 170262 | 22, 29, 21 |
| 21 | MRPL39 | mitochondrial ribosomal protein L39 | 154719 | 154654 | 154721 | 1, 29, 30 |
| 21 | MRPS6 | mitochondrial ribosomal protein S6 | 243927 | 241837 | 198743 | 18, 41, 11 |
| 21 | MORC3 | MORC family CW-type zinc finger 3 | 159256 | 142197 | 159259 | 18, 42, 4 |
| 21 | MX1 | MX dynamin like GTPase 1 | 157601 | 183486 | 184012 | 36, 4, 41 |
| 21 | MX2 | MX dynamin like GTPase 2 | 183486 | 183844 | 157601 | 39, 36, 22 |
| 21 | N6AMT1 | N-6 adenine-specific DNA methyltransferase 1 (putative) | 156239 | 154736 | 198862 | 28, 45, 36 |
| 21 | NDUFV3 | NADH:ubiquinone oxidoreductase subunit V3 | 160194 | 160193 | 160199 | 45, 9, 15 |
| 21 | NCAM2 | neural cell adhesion molecule 2 | 154654 | 154646 | 154719 | 26, 33, 7 |
| 21 | NRIP1 | nuclear receptor interacting protein 1 | 180530 | 243440 | 155313 | 45, 29, 1 |
| 21 | OLIG1 | oligodendrocyte transcription factor 1 | 184221 | 205927 | 159110 | |
| 21 | OLIG2 | oligodendrocyte transcription factor 2 | 205927 | 205929 | 184221 | 8, 38, 17 |
| 21 | PAXBP1 | PAX3 and PAX7 binding protein 1 | 159086 | 159082 | 205929 | 1, 22, 39 |
| 21 | PKNOX1 | PBX/knotted 1 homeobox 1 | 160199 | 160194 | 160200 | 18, 4, 13 |
| 21 | PCNT | pericentrin | 160299 | 160298 | 160305 | 18, 31, 2 |
| 21 | PIGP | phosphatidylinositol glycan anchor biosynthesis class P | 185808 | 183145 | 182670 | |
| 21 | PDE9A | phosphodiesterase 9A | 160191 | 160190 | 160193 | 33, 16, 26 |
| 21 | PFKL | phosphofructokinase, liver type | 141959 | 160224 | 160226 | 21, 9, 35 |
| 21 | GART | phosphoribosylglycinamide formyltransferase, phosphoribosylglycinamide | 159131 | 177692 | 159140 | 38, 9, 45 |
| | | synthetase, phosphoribosylaminoimidazole synthetase | | | | |
| 21 | PTTG1IP | pituitary tumor-transforming 1 interacting protein | 183255 | 184900 | 160255 | 35, 45, 17 |
| 21 | PCBP3 | poly(rC) binding protein 3 | 183570 | 173638 | 142156 | 39, 5, 38 |
| 21 | KCNE1 | potassium voltage-gated channel subfamily E regulatory subunit 1 | 180509 | 243627 | 159200 | 42, 26, 30 |
| 21 | KCNE1B | potassium voltage-gated channel subfamily E regulatory subunit 1B | 276289 | 278961 | 274391 | |
| 21 | KCNE2 | potassium voltage-gated channel subfamily E regulatory subunit 2 | 159197 | 198743 | 205670 | 37, 10, 38 |
| 21 | KCNJ15 | potassium voltage-gated channel subfamily J member 15 | 157551 | 198054 | 157554 | 40, 5, 17 |
| 21 | KCNJ6 | potassium voltage-gated channel subfamily J member 6 | 157542 | 157540 | 184029 | 28, 33, 8 |
| 21 | POTED | POTE ankyrin domain family member D | 166351 | 280108 | 188992 | |
| 21 | PRDM15 | PR/SET domain 15 | 141956 | 183421 | 157617 | |
| 21 | PSMG1 | proteasome assembly chaperone 1 | 183527 | 157557 | 185658 | 9, 25, 14 |
| 21 | PRMT2 | protein arginine methyltransferase 2 | 160310 | 160307 | 183036 | 34, 26, 15 |
| 21 | POFUT2 | protein O-fucosyltransferase 2 | 186866 | 197381 | 182871 | 15, 6, 13 |
| 21 | PCP4 | Purkinje cell protein 4 | 183036 | 183067 | 171587 | 29, 14, 10 |
| 21 | PWP2 | PWP2 periodic tryptophan protein homolog (yeast) | 241945 | 160218 | 160221 | 43, 3, 25 |
| 21 | PDXK | pyridoxal kinase | 160209 | 160208 | 160213 | 34, 26, 38 |
| 21 | RSPH1 | radial spoke head 1 homolog | 160188 | 160185 | 160190 | 10, 40, 42 |
| 21 | RIPK4 | receptor interacting serine/threonine kinase 4 | 183421 | 184012 | 141956 | 24, 42, 39 |
| 21 | RCAN1 | regulator of calcineurin 1 | 159200 | 180509 | 159212 | 29, 4, 35 |
| 21 | RRP1 | ribosomal RNA processing 1 | 160214 | 160213 | 160216 | 13, 9, 4 |
| 21 | RRP1B | ribosomal RNA processing 1B | 160208 | 234289 | 160209 | 22, 9, 13 |
| 21 | RIPPLY3 | ripply transcriptional repressor 3 | 183145 | 159267 | 185808 | |
| 21 | RBM11 | RNA binding motif protein 11 | 185272 | 188992 | 155304 | 6, 25, 8 |
| 21 | RUNX1 | runt related transcription factor 1 | 159216 | 159212 | 280170 | |
| 21 | RWDD2B | RWD domain containing 2B | 156253 | 198862 | 156256 | 28, 15, 17 |
| 21 | S100B | S100 calcium binding protein B | 160307 | 160305 | 160310 | 11, 35, 8 |
| 21 | SIK1 | salt inducible kinase 1 | 142178 | 160202 | 160207 | 18, 29, 2 |
| 21 | SAMSN1 | SAM domain, SH3 domain and nuclear localization signals 1 | 155307 | 155304 | 243440 | 7, 34, 5 |
| 21 | SETD4 | SET domain containing 4 | 185917 | 280170 | 159228 | 39, 13, 30 |
| 21 | SH3BGR | SH3 domain binding glutamate rich protein | 185437 | 157578 | 183778 | 25, 10, 6 |
| 21 | SIM2 | single-minded family bHLH transcription factor 2 | 159263 | 159261 | 159267 | 27, 16, 30 |
| 21 | SMIM11A | small integral membrane protein 11A | 205670 | 159197 | 222018 | |
| 21 | SMIM11B | small integral membrane protein 11B | 273590 | 276076 | 278961 | 18, 34, 5 |
| 21 | SUMO3 | small ubiquitin-like modifier 3 | 184900 | 184787 | 183255 | 27, 3, 13 |
| 21 | SLC19A1 | solute carrier family 19 member 1 | 173638 | 182871 | 183570 | |
| 21 | SLC37A1 | solute carrier family 37 member 1 | 160190 | 160188 | 160191 | 37, 33, 16 |
| 21 | SLC5A3 | solute carrier family 5 member 3 | 198743 | 243927 | 159197 | 18, 41, 11 |
| 21 | SON | SON DNA binding protein | 159140 | 159131 | 159147 | 31, 29, 19 |
| 21 | SPATC1L | spermatogenesis and centriole associated 1 like | 160284 | 160282 | 160285 | 21, 16, 7 |
| 21 | SCAF4 | SR-related CTD associated factor 4 | 156304 | 142168 | 142149 | 22, 18, 38 |
| 21 | SOD1 | superoxide dismutase 1 | 142168 | 156299 | 156304 | 11, 28, 34 |
| 21 | SYNJ1 | synaptojanin 1 | 159082 | 159079 | 159086 | 31, 32, 8 |
| 21 | TIAM1 | T-cell lymphoma invasion and metastasis 1 | 156299 | 206102 | 142168 | 26, 30, 40 |
| 21 | TCP10L | t-complex 10-like | 242220 | 166979 | 265590 | 38, 22, 27 |
| 21 | TTC3 | tetratricopeptide repeat domain 3 | 182670 | 185808 | 157538 | 11, 14, 44 |
| 21 | TSPEAR | thrombospondin type laminin G domain and EAR repeats | 175894 | 160233 | 215455 | 38, 20, |
| 21 | TRAPPC10 | trafficking protein particle complex 10 | 160218 | 160216 | 241945 | 21, 22, 26 |
| 21 | TRPM2 | transient receptor potential cation channel subfamily M member 2 | 142185 | 160226 | 160233 | 3, 26, 6 |
| 21 | TPTE | transmembrane phosphatase with tensin homology | 274391 | 276289 | 277282 | 38, 2, |
| 21 | TMPRSS15 | transmembrane protease, serine 15 | 154646 | 154645 | 154654 | 23, 22, 37 |
| 21 | TMPRSS2 | transmembrane protease, serine 2 | 184012 | 157601 | 183421 | 33, 4, 37 |
| 21 | TMPRSS3 | transmembrane protease, serine 3 | 160183 | 160182 | 160185 | 42, 4, 9 |
| 21 | TMEM50B | transmembrane protein 50B | 142188 | 159128 | 177692 | 40, 11, 31 |
| 21 | TFF1 | trefoil factor 1 | 160182 | 160181 | 160183 | 37, 23, 16 |
| 21 | TFF2 | trefoil factor 2 | 160181 | 160180 | 160182 | 37, 10, 18 |
| 21 | TFF3 | trefoil factor 3 | 160180 | 160179 | 160181 | 40, 16, 15 |
| 21 | WRB | tryptophan rich basic protein | 182093 | 205581 | 157578 | |
| 21 | U2AF1 | U2 small nuclear RNA auxiliary factor 1 | 160201 | 160200 | 160202 | 12, 4, 19 |
| 21 | U2AF1L5 | U2 small nuclear RNA auxiliary factor 1 like 5 | 275895 | 274276 | 276076 | |
| 21 | UBASH3A | ubiquitin associated and SH3 domain containing A | 160185 | 160183 | 160188 | 36, 5, 39 |
| 21 | UBE2G2 | ubiquitin conjugating enzyme E2 G2 | 184787 | 189169 | 184900 | 43, 6, 14 |
| 21 | USP16 | ubiquitin specific peptidase 16 | 156256 | 156253 | 156261 | 45, 17, 29 |
| 21 | USP25 | ubiquitin specific peptidase 25 | 155313 | 180530 | 154639 | 43, 40, 37 |
| 21 | URB1 | URB1 ribosome biogenesis 1 homolog (S. cerevisiae) | 142207 | 170262 | 166979 | 3, 21, 40 |
| 21 | UMODL1 | uromodulin like 1 | 177398 | 173276 | 160179 | 39, |
| 21 | WDR4 | WD repeat domain 4 | 160193 | 160191 | 160194 | 41, 36, 13 |
| 21 | YBEY | ybeY metallopeptidase (putative) | 182362 | 160294 | 160298 | 43, 15, 37 |
| 21 | ZBTB21 | zinc finger and BTB domain containing 21 | 173276 | 157617 | 177398 | 42, 39, 32 |
| 22 | ACO2 | aconitase 2 | 100412 | 100410 | 100413 | 40, 1, 21 |
| 22 | ACR | acrosin | 100312 | 251322 | 079974 | 38, 27, 8 |
| 22 | ASCC2 | activating signal cointegrator 1 complex subunit 2 | 100325 | 184076 | 100330 | 24, 27, 4 |
| 22 | ATF4 | activating transcription factor 4 | 128272 | 100335 | 187051 | 13, 18, 21 |
| 22 | AP1B1 | adaptor related protein complex 1 beta 1 subunit | 100280 | 100276 | 128250 | 21, 38, 40 |
| 22 | ADORA2A | adenosine A2a receptor | 128271 | 258555 | 100024 | |
| 22 | ADSL | adenylosuccinate lyase | 239900 | 100354 | 100359 | 34, 31, 1 |
| 22 | ARFGAP3 | ADP ribosylation factor GTPase activating protein 3 | 242247 | 128274 | 100266 | 18, 38, 13 |
| 22 | ADM2 | adrenomedullin 2 | 128165 | 100241 | 100253 | 18, 5, 45 |
| 22 | ALG12 | ALG12, alpha-1,6-mannosyltransferase | 182858 | 100426 | 184164 | 9, 1, 40 |
| 22 | A4GALT | alpha 1,4-galactosyltransferase | 128274 | 249222 | 242247 | 36, 41, 4 |
| 22 | NAGA | alpha-N-acetylgalactosaminidase | 198951 | 183066 | 177096 | 29, 32, 6 |
| 22 | ANKRD54 | ankyrin repeat domain 54 | 100124 | 128310 | 100129 | 9, 31, 41 |
| 22 | APOBEC3A | apolipoprotein B mRNA editing enzyme catalytic subunit 3A | 128383 | 183741 | 179750 | |
| 22 | APOBEC3B | apolipoprotein B mRNA editing enzyme catalytic subunit 3B | 179750 | 128383 | 244509 | 18, 11, 5 |
| 22 | APOBEC3C | apolipoprotein B mRNA editing enzyme catalytic subunit 3C | 244509 | 179750 | 243811 | 18, 2, 21 |
| 22 | APOBEC3D | apolipoprotein B mRNA editing enzyme catalytic subunit 3D | 243811 | 244509 | 128394 | 40, 18, 36 |
| 22 | APOBEC3F | apolipoprotein B mRNA editing enzyme catalytic subunit 3F | 128394 | 243811 | 239713 | 41, 29, 22 |
| 22 | APOBEC3G | apolipoprotein B mRNA editing enzyme catalytic subunit 3G | 239713 | 128394 | 100298 | 41, 5, 21 |
| 22 | APOBEC3H | apolipoprotein B mRNA editing enzyme catalytic subunit 3H | 100298 | 239713 | 100307 | 5, 40, 32 |
| 22 | APOL1 | apolipoprotein L1 | 100342 | 128335 | 100345 | 36, 37, 39 |
| 22 | APOL2 | apolipoprotein L2 | 128335 | 100336 | 100342 | 39, 34, 36 |
| 22 | APOL3 | apolipoprotein L3 | 128284 | 100320 | 100336 | 36, 10, 15 |
| 22 | APOL4 | apolipoprotein L4 | 100336 | 128284 | 128335 | 42, 33, 36 |
| 22 | APOL5 | apolipoprotein L5 | 128313 | 221963 | 100320 | 23, 38, 32 |
| 22 | APOL6 | apolipoprotein L6 | 221963 | 198125 | 128313 | 36, 13, 21 |
| 22 | AIFM3 | apoptosis inducing factor, mitochondria associated 3 | 183773 | 099942 | 099949 | |
| 22 | ARVCF | armadillo repeat gene deleted in velocardiofacial syndrome | 099889 | 093010 | 183597 | 29, 33, 15 |
| 22 | ARSA | arylsulfatase A | 100299 | 008735 | 251322 | 40, 20, |
| 22 | ASPHD2 | aspartate beta-hydroxylase domain containing 2 | 128203 | 100095 | 100099 | 26, 14, 36 |
| 22 | DRICH1 | aspartate rich 1 | 189269 | 128322 | 159496 | |
| 22 | ATXN10 | ataxin 10 | 130638 | 077942 | 188064 | 41, 13, 21 |
| 22 | ATP5L2 | ATP synthase, H+ transporting, mitochondrial Fo complex subunit G2 | 249222 | 100243 | 128274 | 6, 17, |
| 22 | ATP6V1E1 | ATPase H+ transporting V1 subunit E1 | 131100 | 182902 | 099968 | 41, 34, 30 |
| 22 | BAGE5 | B melanoma antigen family member 5 | 279973 | 280363 | 130538 | |
| 22 | BAIAP2L2 | BAI1 associated protein 2 like 2 | 128298 | 100156 | 184381 | 16, 28, 23 |
| 22 | BIK | BCL2 interacting killer | 100290 | 100271 | 100294 | 21, 41, 22 |
| 22 | BCL2L13 | BCL2 like 13 | 099968 | 131100 | 015475 | |
| 22 | BCR | BCR, RhoGEF and GTPase activating protein | 186716 | 100228 | 128322 | 43, 1, 21 |
| 22 | UPB1 | beta-ureidopropionase 1 | 100024 | 128271 | 138867 | 19, 17, 26 |
| 22 | BID | BH3 interacting domain death agonist | 015475 | 099968 | 243156 | 43, 6, 3 |
| 22 | BPIFC | BPI fold containing family C | 184459 | 100220 | 100225 | 13, 35, 28 |
| 22 | BRD1 | bromodomain containing 1 | 100425 | 219438 | 100426 | 38, 26, 33 |
| 22 | C1QTNF6 | C1q and tumor necrosis factor related protein 6 | 133466 | 100385 | 278195 | |
| 22 | CELSR1 | cadherin EGF LAG seven-pass G-type receptor 1 | 075275 | 100416 | 075240 | 34, 40, 30 |
| 22 | CABIN1 | calcineurin binding protein 1 | 099991 | 099984 | 099994 | |
| 22 | CABP7 | calcium binding protein 7 | 100314 | 186575 | 100319 | 11, 41, 29 |
| 22 | CACNG2 | calcium voltage-gated channel auxiliary subunit gamma 2 | 166862 | 100353 | 100360 | 7, 14, 8 |
| 22 | CACNA1I | calcium voltage-gated channel subunit alpha1 I | 100346 | 187051 | 176177 | 26, 40, 25 |
| 22 | CPT1B | carnitine palmitoyltransferase 1B | 205560 | 217442 | 254413 | 15, 38, 24 |
| 22 | CSNK1E | casein kinase 1 epsilon | 213923 | 198792 | 168135 | |
| 22 | CARD10 | caspase recruitment domain family member 10 | 100065 | 100060 | 128283 | 17, 28, 26 |
| 22 | CECR1 | cat eye syndrome chromosome region, candidate 1 | 093072 | 069998 | 099954 | 39, 42, 10 |
| 22 | CECR5 | cat eye syndrome chromosome region, candidate 5 | 069998 | 183307 | 093072 | 33, 5, 9 |
| 22 | CECR6 | cat eye syndrome chromosome region, candidate 6 | 183307 | 177663 | 069998 | 5, 10, 14 |
| 22 | COMT | catechol-O-methyltransferase | 093010 | 184470 | 099889 | 35, 41, 32 |
| 22 | CDC42EP1 | CDC42 effector protein 1 | 128283 | 100065 | 100079 | 3, 6, 37 |
| 22 | CECR2 | CECR2, histone acetyl-lysine reader | 099954 | 093072 | 182902 | 18, 2, 32 |
| 22 | CDC45 | cell division cycle 45 | 093009 | 070010 | 184113 | 5, 39, 7 |
| 22 | CENPM | centromere protein M | 100162 | 159958 | 100167 | 21, 41, 35 |
| 22 | CERK | ceramide kinase | 100422 | 075240 | 054611 | 22, 43, 29 |
| 22 | CCT8L2 | chaperonin containing TCP1 subunit 8 like 2 | 198445 | 198062 | 172967 | 38, 17, 8 |
| 22 | CHEK2 | checkpoint kinase 2 | 183765 | 100154 | 100209 | 10, 9, 5 |
| 22 | CBY1 | chibby family member 1, beta catenin antagonist | 100211 | 184949 | 100216 | 23, 30, 9 |
| 22 | CHKB-CPT1B | CHKB-CPT1 Breadthrough | 254413 | 205560 | 100288 | |
| 22 | CHKB | choline kinase beta | 100288 | 254413 | 008735 | 7, 11, 31 |
| 22 | CHADL | chondroadherin like | 100399 | 100395 | 100401 | 26, 31, 8 |
| 22 | CBX6 | chromobox 6 | 183741 | 221890 | 128383 | 26, 34, 1 |
| 22 | CBX7 | chromobox 7 | 100307 | 100298 | 100311 | 26, 14, 28 |
| 22 | C22orf15 | chromosome 22 open reading frame 15 | 169314 | 128218 | 250479 | 42, 39, 30 |
| 22 | C22orf23 | chromosome 22 open reading frame 23 | 128346 | 100139 | 100142 | 38, 30, 37 |
| 22 | C22orf29 | chromosome 22 open reading frame 29 | 215012 | 185838 | 184470 | 40, 43, 11 |
| 22 | C22orf31 | chromosome 22 open reading frame 31 | 100249 | 183579 | 183762 | 29, 20, 38 |
| 22 | C22orf39 | chromosome 22 open reading frame 39 | 242259 | 100084 | 185608 | 29, 33, 26 |
| 22 | C22orf42 | chromosome 22 open reading frame 42 | 205856 | 100170 | 128253 | 28, 38, |
| 22 | C22orf46 | chromosome 22 open reading frame 46 | 184208 | 100138 | 167077 | 40, 18, 34 |
| 22 | CLTCL1 | clathrin heavy chain like 1 | 070371 | 100075 | 100084 | 26, 31, 38 |
| 22 | CLDN5 | claudin 5 | 184113 | 093009 | 184702 | 1, 26, 20 |
| 22 | CCDC116 | coiled-coil domain containing 116 | 161180 | 161179 | 128228 | 38, 10, 8 |
| 22 | CCDC117 | coiled-coil domain containing 117 | 159873 | 100209 | 100219 | 43, 22, 38 |
| 22 | CCDC134 | coiled-coil domain containing 134 | 100147 | 167077 | 198911 | 2, 6, 18 |
| 22 | CCDC157 | coiled-coil domain containing 157 | 187860 | 099995 | 099999 | 38, 26, 39 |
| 22 | CCDC188 | coiled-coil domain containing 188 | 234409 | 099904 | 040608 | 38, 28, |
| 22 | CHCHD10 | coiled-coil-helix-coiled-coil-helix domain containing 10 | 250479 | 169314 | 099953 | 25, 28, 17 |
| 22 | CSDC2 | cold shock domain containing C2 | 172346 | 100413 | 100417 | 14, 8, 17 |
| 22 | CSF2RB | colony stimulating factor 2 receptor beta common subunit | 100368 | 100365 | 185264 | 1, 32, 22 |
| 22 | CRKL | CRK like proto-oncogene, adaptor protein | 099942 | 099940 | 183773 | 37, 7, 29 |
| 22 | CRYBA4 | crystallin beta A4 | 196431 | 100122 | 169184 | 14, 12, 32 |
| 22 | CRYBB1 | crystallin beta B1 | 100122 | 128294 | 196431 | 14, 10, 22 |
| 22 | CRYBB2 | crystallin beta B2 | 244752 | 100053 | 100068 | 14, 20, 38 |
| 22 | CRYBB3 | crystallin beta B3 | 100053 | 197077 | 244752 | 14, 33, 35 |
| 22 | CDPF1 | cysteine rich DPF motif domain containing 1 | 205643 | 186951 | 130943 | 27, 5, 3 |
| 22 | CRELD2 | cysteine rich with EGF like domains 2 | 184164 | 182858 | 198355 | 41, 25, 6 |
| 22 | CYB5R3 | cytochrome b5 reductase 3 | 100243 | 100227 | 249222 | 1, 26, 2 |
| 22 | CYP2D6 | cytochrome P450 family 2 subfamily D member 6 | 100197 | 184983 | 205702 | |
| 22 | CYP2D7 | cytochrome P450 family 2 subfamily D member 7 (gene/pseudogene) | 205702 | 100197 | 100207 | 19, 39, 35 |
| 22 | CYTH4 | cytohesin 4 | 100055 | 128340 | 166897 | 39, 5, 36 |
| 22 | DDT | D-dopachrome tautomerase | 099977 | 099974 | 099984 | 15, 19, 8 |
| 22 | DDTL | D-dopachrome tautomerase-like | 099974 | 133433 | 099977 | 36, 1, 39 |
| 22 | DDX17 | DEAD-box helicase 17 | 100201 | 100196 | 100206 | 31, 11, 40 |
| 22 | DENND6B | DENN domain containing 6B | 205593 | 196576 | 100239 | 26, 41, 38 |
| 22 | DEPDC5 | DEP domain containing 5 | 100150 | 183530 | 128245 | 22, 13, 43 |
| 22 | DERL3 | derlin 3 | 099958 | 099956 | 133460 | 22, 20, 41 |
| 22 | DESI1 | desumoylating isopeptidase 1 | 100418 | 100417 | 196419 | 9, 26, 15 |
| 22 | DRG1 | developmentally regulated GTP binding protein 1 | 185721 | 100105 | 184708 | 43, 38, 9 |
| 22 | DGCR8 | DGCR8, microprocessor complex subunit | 128191 | 183597 | 099899 | 1, 37, 13 |
| 22 | DGCR14 | DiGeorge syndrome critical region gene 14 | 100056 | 070413 | 206203 | 34, 31, 32 |
| 22 | DGCR2 | DiGeorge syndrome critical region gene 2 | 070413 | 279560 | 100056 | 8, 42, 38 |
| 22 | DGCR6 | DiGeorge syndrome critical region gene 6 | 183628 | 275793 | 100033 | 25, 2, 8 |
| 22 | DGCR6L | DiGeorge syndrome critical region gene 6-like | 128185 | 040608 | 161133 | 34, 29, 25 |
| 22 | DMC1 | DNA meiotic recombinase 1 | 100206 | 100201 | 184949 | 45, 39, 22 |
| 22 | POLDIP3 | DNA polymerase delta interacting protein 3 | 100227 | 183569 | 100243 | 39, 42, 24 |
| 22 | DNAJB7 | DnaJ heat shock protein family (Hsp40) member B7 | 172404 | 196236 | 100387 | 38, 40, 22 |
| 22 | DUSP18 | dual specificity phosphatase 18 | 167065 | 100036 | 184792 | |
| 22 | DNAL4 | dynein axonemal light chain 4 | 100246 | 100242 | 221890 | 1, 27, 26 |
| 22 | EP300 | E1A binding protein p300 | 100393 | 100387 | 100395 | 18, 22, 5 |
| 22 | EFCAB6 | EF-hand calcium binding domain 6 | 186976 | 186732 | 130540 | 38, 31, 40 |
| 22 | EMID1 | EMI domain containing 1 | 186998 | 183762 | 100263 | 36, 29, 33 |
| 22 | ENTHD1 | ENTH domain containing 1 | 176177 | 100346 | 100351 | 38, 7, 35 |
| 22 | EIF3D | eukaryotic translation initiation factor 3 subunit D | 100353 | 100350 | 166862 | 21, 3, 13 |
| 22 | EIF3L | eukaryotic translation initiation factor 3 subunit L | 100129 | 100124 | 100139 | 13, 39, 2 |
| 22 | EIF4ENIF1 | eukaryotic translation initiation factor 4E nuclear import factor 1 | 184708 | 185721 | 198089 | |
| 22 | EWSR1 | EWS RNA binding protein 1 | 182944 | 100263 | 185340 | 41, 21, 39 |
| 22 | ELFN2 | extracellular leucine rich repeat and fibronectin type III domain containing 2 | 166897 | 100055 | 100060 | 22, 28, 38 |
| 22 | FOXRED2 | FAD dependent oxidoreductase domain containing 2 | 100350 | 100348 | 100353 | 28, 9, 8 |
| 22 | FAM109B | family with sequence similarity 109 member B | 177096 | 198951 | 183172 | 15, 1, 32 |
| 22 | FAM118A | family with sequence similarity 118 member A | 100376 | 100373 | 077935 | |
| 22 | FAM19A5 | family with sequence similarity 19 member A5, C-C motif chemokine like | 219438 | 278881 | 100425 | 11, 8, 14 |
| 22 | FAM227A | family with sequence similarity 227 member A | 184949 | 100206 | 100211 | 14, 7, 38 |
| 22 | FAM83F | family with sequence similarity 83 member F | 133477 | 100351 | 100354 | 22, 28, 3 |
| 22 | FBXO7 | F-box protein 7 | 100225 | 184459 | 185666 | 25, 9, 32 |
| 22 | FBLN1 | fibulin 1 | 077942 | 128408 | 130638 | 32, 34, 27 |
| 22 | GNAZ | G protein subunit alpha z | 128266 | 100218 | 100228 | 8, 26, 14 |
| 22 | GNB1L | G protein subunit beta 1 like | 185838 | 184058 | 215012 | 40, 43, 11 |
| 22 | GRK3 | G protein-coupled receptor kinase 3 | 100077 | 100068 | 133454 | 22, 26, 28 |
| 22 | GTSE1 | G2 and S-phase expressed 1 | 075218 | 075234 | 100416 | 13, 39, 22 |
| 22 | GAL3ST1 | galactose-3-O-sulfotransferase 1 | 128242 | 214491 | 100029 | 17, 8, 3 |
| 22 | GALR3 | galanin receptor 3 | 128310 | 100116 | 100124 | 27, 14, |
| 22 | LGALS1 | galectin 1 | 100097 | 241360 | 273899 | 43, 34, 45 |
| 22 | LGALS2 | galectin 2 | 100079 | 128283 | 100083 | 10, 17, 30 |
| 22 | GGT1 | gamma-glutamyltransferase 1 | 100031 | 100028 | 178026 | 5, 23, 24 |
| 22 | GGT2 | gamma-glutamyltransferase 2 | 133475 | 187905 | 274600 | |
| 22 | GGT5 | gamma-glutamyltransferase 5 | 099998 | 099994 | 100014 | 2, 36, 45 |
| 22 | GGTLC2 | gamma-glutamyltransferase light chain 2 | 100121 | 211657 | 211658 | 38, 28, |
| 22 | GGTLC3 | Gamma-glutamyltransferase light chain 3 | 274252 | 184979 | 278558 | |
| 22 | GATSL3 | GATS protein like 3 | 239282 | 099985 | 248751 | 13, 44, 38 |
| 22 | GSTT2 | glutathione S-transferase theta 2 (gene/pseudogene) | 099984 | 099977 | 099991 | 34, 2, 3 |
| 22 | GSTT2B | glutathione S-transferase theta 2B (gene/pseudogene) | 133433 | 240972 | 099974 | 2, 15, 29 |
| 22 | GOAT | glycine C-acetyltransferase | 100116 | 189060 | 128310 | 29, 40, 26 |
| 22 | GP1BB | glycoprotein lb platelet beta subunit | 203618 | 184702 | 184058 | 31, 36, 28 |
| 22 | GGA1 | golgi associated, gamma adaptin ear containing, ARF binding protein 1 | 100083 | 100079 | 100092 | 3, 38, 36 |
| 22 | GSC2 | goosecoid homeobox 2 | 063515 | 206203 | 100075 | 38, |
| 22 | GRAMD4 | GRAM domain containing 4 | 075240 | 075275 | 100422 | 22, 20, 17 |
| 22 | GAB4 | GRB2 associated binding protein family member 4 | 215568 | 172967 | 177663 | 38, |
| 22 | GRAP2 | GRB2-related adaptor protein 2 | 100351 | 176177 | 133477 | 39, 13, 7 |
| 22 | GAS2L1 | growth arrest specific 2 like 1 | 185340 | 182944 | 100276 | 38, 5, 13 |
| 22 | GTPBP1 | GTP binding protein 1 | 100226 | 100221 | 100242 | 22, 13, 43 |
| 22 | GUCD1 | guanylyl cyclase domain containing 1 | 138867 | 100024 | 100028 | 3, 9, 34 |
| 22 | H1F0 | H1 histone family member 0 | 189060 | 100106 | 100116 | 13, 1, 6 |
| 22 | HMOX1 | heme oxygenase 1 | 100292 | 100284 | 100297 | 36, 32, 34 |
| 22 | HIC2 | HIC ZBTB transcriptional repressor 2 | 169635 | 274600 | 206140 | 1, 32, 15 |
| 22 | HIRA | histone cell cycle regulator | 100084 | 070371 | 242259 | 3, 24, 40 |
| 22 | HDAC10 | histone deacetylase 10 | 100429 | 128159 | 188130 | 9, 22, 5 |
| 22 | HMGXB4 | HMG-box containing 4 | 100281 | 175329 | 100284 | 19, 12, 6 |
| 22 | HORMAD2 | HORMA domain containing 2 | 176635 | 100330 | 128342 | 38, 19, 28 |
| 22 | HPS4 | HPS4, biogenesis of lysosomal organelles complex 3 subunit 2 | 100099 | 128203 | 100104 | 22, 39, 43 |
| 22 | HSCB | HscB mitochondrial iron-sulfur cluster cochaperone | 100209 | 183765 | 159873 | 40, 21, 17 |
| 22 | IGLC1 | Immunoglobulin lambda constant 1 | 211675 | 211674 | 211676 | 30, 41, 7 |
| 22 | IGLC2 | Immunoglobulin lambda constant 2 | 211677 | 211676 | 211678 | |
| 22 | IGLC3 | Immunoglobulin lambda constant 3 (Kern-Oz+ marker) | 211679 | 211678 | 211680 | |
| 22 | IGLC7 | Immunoglobulin lambda constant 7 | 211685 | 211684 | 100218 | |
| 22 | IGLJ1 | Immunoglobulin lambda joining 1 | 211674 | 254709 | 211675 | |
| 22 | IGLJ2 | Immunoglobulin lambda joining 2 | 211676 | 211675 | 211677 | |
| 22 | IGLJ3 | Immunoglobulin lambda joining 3 | 211678 | 211677 | 211679 | |
| 22 | IGLJ4 | Immunoglobulin lambda joining 4 (non-functional) | 211680 | 211679 | 211681 | |
| 22 | IGLJ5 | Immunoglobulin lambda joining 5 (non-functional) | 211681 | 211680 | 211682 | |
| 22 | IGLJ6 | Immunoglobulin lambda joining 6 | 211682 | 211681 | 211684 | |
| 22 | IGLJ7 | Immunoglobulin lambda joining 7 | 211684 | 211682 | 211685 | |
| 22 | IGLL1 | immunoglobulin lambda like polypeptide 1 | 128322 | 186716 | 189269 | 7, 5, 15 |
| 22 | IGLL5 | immunoglobulin lambda like polypeptide 5 | 254709 | 211673 | 211674 | |
| 22 | IGLV10-54 | Immunoglobulin lambda variable 10-54 | 211642 | 211641 | 169575 | |
| 22 | IGLV11-55 | Immunoglobulin lambda variable 11-55 (non-functional) | 211641 | 211640 | 211642 | |
| 22 | IGLV1-36 | Immunoglobulin lambda variable 1-36 | 211655 | 211654 | 275004 | |
| 22 | IGLV1-40 | Immunoglobulin lambda variable 1-40 | 211653 | 211652 | 211654 | |
| 22 | IGLV1-44 | Immunoglobulin lambda variable 1-44 | 211651 | 211650 | 211652 | 30, 41, 22 |
| 22 | IGLV1-47 | Immunoglobulin lambda variable 1-47 | 211648 | 211647 | 211649 | |
| 22 | IGLV1-50 | Immunoglobulin lambda variable 1-50 (non-functional) | 211645 | 211644 | 223350 | |
| 22 | IGLV1-51 | Immunoglobulin lambda variable 1-51 | 211644 | 211643 | 211645 | |
| 22 | IGLV2-11 | Immunoglobulin lambda variable 2-11 | 211668 | 211667 | 211669 | |
| 22 | IGLV2-14 | Immunoglobulin lambda variable 2-14 | 211666 | 211665 | 211667 | |
| 22 | IGLV2-18 | Immunoglobulin lambda variable 2-18 | 211664 | 211663 | 211665 | |
| 22 | IGLV2-23 | Immunoglobulin lambda variable 2-23 | 211660 | 211659 | 211661 | |
| 22 | IGLV2-33 | Immunoglobulin lambda variable 2-33 (non-functional) | 211656 | 185686 | 211657 | |
| 22 | IGLV2-8 | Immunoglobulin lambda variable 2-8 | 278196 | 211670 | 211672 | |
| 22 | IGLV3-1 | Immunoglobulin lambda variable 3-1 | 211673 | 211672 | 254709 | |
| 22 | IGLV3-10 | Immunoglobulin lambda variable 3-10 | 211669 | 211668 | 211670 | |
| 22 | IGLV3-12 | Immunoglobulin lambda variable 3-12 | 211667 | 211666 | 211668 | |
| 22 | IGLV3-16 | Immunoglobulin lambda variable 3-16 | 211665 | 211664 | 211666 | |
| 22 | IGLV3-19 | Immunoglobulin lambda variable 3-19 | 211663 | 211662 | 211664 | |
| 22 | IGLV3-21 | Immunoglobulin lambda variable 3-21 | 211662 | 211661 | 211663 | |
| 22 | IGLV3-22 | Immunoglobulin lambda variable 3-22 (gene/pseudogene) | 211661 | 211660 | 211662 | |
| 22 | IGLV3-25 | Immunoglobulin lambda variable 3-25 | 211659 | 211658 | 211660 | |
| 22 | IGLV3-27 | Immunoglobulin lambda variable 3-27 | 211658 | 100121 | 211659 | |
| 22 | IGLV3-32 | Immunoglobulin lambda variable 3-32 (non-functional) | 211657 | 211656 | 100121 | |
| 22 | IGLV3-9 | Immunoglobulin lambda variable 3-9 (gene/pseudogene) | 211670 | 211669 | 278196 | |
| 22 | IGLV4-3 | Immunoglobulin lambda variable 4-3 | 211672 | 278196 | 211673 | |
| 22 | IGLV4-60 | Immunoglobulin lambda variable 4-60 | 211639 | 211638 | 211640 | |
| 22 | IGLV4-69 | Immunoglobulin lambda variable 4-69 | 211637 | 100038 | 211638 | |
| 22 | IGLV5-37 | Immunoglobulin lambda variable 5-37 | 211654 | 211653 | 211655 | |
| 22 | IGLV5-45 | Immunoglobulin lambda variable 5-45 | 211650 | 211649 | 211651 | |
| 22 | IGLV5-48 | Immunoglobulin lambda variable 5-48 (non-functional) | 211647 | 223350 | 211648 | |
| 22 | IGLV5-52 | Immunoglobulin lambda variable 5-52 | 211643 | 169575 | 211644 | |
| 22 | IGLV6-57 | Immunoglobulin lambda variable 6-57 | 211640 | 211639 | 211641 | 30, 24, 36 |
| 22 | IGLV7-43 | Immunoglobulin lambda variable 7-43 | 211652 | 211651 | 211653 | |
| 22 | IGLV7-46 | Immunoglobulin lambda variable 7-46 (gene/pseudogene) | 211649 | 211648 | 211650 | |
| 22 | IGLV8-61 | Immunoglobulin lambda variable 8-61 | 211638 | 211637 | 211639 | |
| 22 | IGLV9-49 | Immunoglobulin lambda variable 9-49 | 223350 | 211645 | 211647 | |
| 22 | INPP5J | inositol polyphosphate-5-phosphatase J | 185133 | 198832 | 100078 | 40, 29, 24 |
| 22 | IL17RA | interleukin 17 receptor A | 177663 | 215568 | 183307 | 5, 40, 31 |
| 22 | IL17REL | interleukin 17 receptor E like | 188263 | 198355 | 138892 | 36, 38, |
| 22 | IL2RB | interleukin 2 receptor subunit beta | 100385 | 187045 | 133466 | 42, 36, 32 |
| 22 | ISX | intestine specific homeobox | 175329 | 133424 | 100281 | 19, 16, |
| 22 | IFT27 | intraflagellar transport 27 | 100360 | 166862 | 100362 | 26, 29, 40 |
| 22 | JOSD1 | Josephin domain containing 1 | 100221 | 100216 | 100226 | 4, 34, 1 |
| 22 | KDELR3 | KDEL endoplasmic reticulum protein retention receptor 3 | 100196 | 168135 | 100201 | 7, 9, 6 |
| 22 | KLHDC7B | kelch domain containing 7B | 130487 | 177989 | 217442 | 29, 23, 9 |
| 22 | KLHL22 | kelch like family member 22 | 099910 | 277971 | 099917 | 18, 1, 34 |
| 22 | KIAA0930 | KIAA0930 | 100364 | 093000 | 100373 | 26, 5, 34 |
| 22 | KIAA1644 | KIAA1644 | 138944 | 138964 | 188636 | 44, 14, 6 |
| 22 | KIAA1671 | KIAA1671 | 197077 | 206069 | 100053 | 43, 24, 39 |
| 22 | KREMEN1 | kringle containing transmembrane protein 1 | 183762 | 100249 | 186998 | 24, 35, 1 |
| 22 | L3MBTL2 | L3MBTL2 polycomb repressive complex 1 subunit | 100395 | 100393 | 100399 | 41, 35, 22 |
| 22 | LARGE1 | LARGE xylosyl- and glucuronyltransferase 1 | 133424 | 100234 | 175329 | |
| 22 | LRP5L | LDL receptor related protein 5 like | 100068 | 244752 | 100077 | 39, 25, 5 |
| 22 | LRRC74B | leucine rich repeat containing 74B | 187905 | 099960 | 133475 | |
| 22 | LRRC75B | leucine rich repeat containing 75B | 178026 | 100031 | 184571 | |
| 22 | LDOC1L | leucine zipper down-regulated in cancer 1 like | 188636 | 138944 | 186654 | 11, 42, 44 |
| 22 | LZTR1 | leucine zipper like transcription regulator 1 | 099949 | 183773 | 184436 | 1, 40, 18 |
| 22 | LIF | leukemia inhibitory factor | 128342 | 176635 | 099985 | 12, 28, 37 |
| 22 | LIMK2 | LIM domain kinase 2 | 182541 | 138942 | 100100 | 42, 24, 32 |
| 22 | LMF2 | lipase maturation factor 2 | 100258 | 100253 | 025770 | 35, 7, 28 |
| 22 | MIF | macrophage migration inhibitory factor (glycosylation-inhibiting factor) | 240972 | 251357 | 133433 | 3, 21, 12 |
| 22 | MAFF | MAF bZIP transcription factor F | 185022 | 184381 | 198792 | 1, 32, 23 |
| 22 | MCAT | malonyl-CoA-acyl carrier protein transacylase | 100294 | 100290 | 100300 | 27, 20, 3 |
| 22 | MGAT3 | mannosyl (beta-1,4-)-glycoprotein beta-1,4-N-acetylglucosaminyltransferase | 128268 | 100324 | 100335 | 22, 14, 26 |
| 22 | MMP11 | matrix metallopeptidase 11 | 099953 | 250479 | 280178 | 32, 27, 44 |
| 22 | MED15 | mediator complex subunit 15 | 099917 | 099910 | 241973 | 18, 3, 28 |
| 22 | MKL1 | megakaryoblastic leukemia (translocation) 1 | 196588 | 100359 | 128285 | 22, 26, 7 |
| 22 | MLC1 | megalencephalic leukoencephalopathy with subcortical cysts 1 | 100427 | 138892 | 073146 | 8, 7, 26 |
| 22 | MEI1 | meiotic double-stranded break formation protein 1 | 167077 | 184208 | 100147 | 36, 38, 5 |
| 22 | MCHR1 | melanin concentrating hormone receptor 1 | 128285 | 196588 | 100372 | 8, 44, 28 |
| 22 | MPST | mercaptopyruvate sulfurtransferase | 128309 | 128311 | 100379 | 34, 21, 16 |
| 22 | MPPED1 | metallophosphoesterase domain containing 1 | 186732 | 159307 | 186976 | 8, 18, 39 |
| 22 | MFNG | MFNG O-fucosylpeptide 3-beta-N-acetylglucosaminyltransferase | 100060 | 166897 | 100065 | 41, 43, 22 |
| 22 | MICALL1 | MICAL like 1 | 100139 | 100129 | 128346 | 41, 9, 32 |
| 22 | MICAL3 | microtubule associated monooxygenase, calponin and UM domain containing 3 | 243156 | 015475 | 215193 | |
| 22 | MCM5 | minichromosome maintenance complex component 5 | 100297 | 100292 | 100302 | 21, 34, 36 |
| 22 | MIEF1 | mitochondrial elongation factor 1 | 100335 | 128268 | 128272 | |
| 22 | MTFP1 | mitochondrial fission process 1 | 242114 | 249590 | 100012 | 21, 41, 7 |
| 22 | MRPL40 | mitochondrial ribosomal protein L40 | 185608 | 242259 | 070010 | 34, 43, 29 |
| 22 | MAPK1 | mitogen-activated protein kinase 1 | 100030 | 100027 | 100034 | 24, 34, 22 |
| 22 | MAPK11 | mitogen-activated protein kinase 11 | 185386 | 188130 | 196576 | 1, 5, 26 |
| 22 | MAPK12 | mitogen-activated protein kinase 12 | 188130 | 100429 | 185386 | 5, 13, 9 |
| 22 | MAPK8IP2 | mitogen-activated protein kinase 8 interacting protein 2 | 008735 | 100288 | 100299 | 26, 8, 13 |
| 22 | MN1 | MN1 proto-oncogene, transcriptional regulator | 169184 | 196431 | 180957 | 11, 1, 45 |
| 22 | MORC2 | MORC family CW-type zinc finger 2 | 133422 | 184792 | 183963 | 26, 6, 16 |
| 22 | MOV10L1 | Mov10 RISC complex RNA helicase like 1 | 073146 | 100427 | 073150 | 38, 45, 1 |
| 22 | MB | myoglobin | 198125 | 100302 | 221963 | 25, 15, 18 |
| 22 | MIOX | myo-inositol oxygenase | 100253 | 128165 | 100258 | 17, 40, 23 |
| 22 | MYH9 | myosin heavy chain 9 | 100345 | 100342 | 100348 | 18, 40, 16 |
| 22 | MYO18B | myosin XVIIIB | 133454 | 100077 | 100095 | |
| 22 | MTMR3 | myotubularin related protein 3 | 100330 | 100325 | 176635 | 11, 26, 20 |
| 22 | NDUFA6 | NADH:ubiquinone oxidoreductase subunit A6 | 184983 | 183172 | 100197 | 25, 34, 29 |
| 22 | NF2 | neurofibromin 2 | 186575 | 184117 | 100314 | 27, 22, 35 |
| 22 | NEFH | neurofilament heavy | 100285 | 128250 | 100296 | 26, 33, 11 |
| 22 | NPTXR | neuronal pentraxin receptor | 221890 | 100246 | 183741 | 8, 9, 23 |
| 22 | NCF4 | neutrophil cytosolic factor 4 | 100365 | 100362 | 100368 | 5, 41, 7 |
| 22 | NFAM1 | NFAT activating protein with ITAM motif 1 | 235568 | 100207 | 189306 | 5, 36, 6 |
| 22 | NIPSNAP1 | nipsnap homolog 1 | 184117 | 100296 | 186575 | 3, 21, 14 |
| 22 | NCAPH2 | non-SMC condensin II complex subunit H2 | 025770 | 100258 | 130489 | 1, 26, 14 |
| 22 | NOL12 | nucleolar protein 12 | 273899 | 100097 | 100101 | 36, 37, 39 |
| 22 | NUP50 | nudeoporin 50 | 093000 | 056487 | 100364 | |
| 22 | OR11H1 | olfactory receptor family 11 subfamily H member 1 | 130538 | 279973 | 198062 | |
| 22 | OSM | on costatin M | 099985 | 128342 | 239282 | 5, 20, 39 |
| 22 | ODF3B | outer dense fiber of sperm tails 3B | 177989 | 025708 | 130487 | 22, 20, 27 |
| 22 | OSBP2 | oxysterol binding protein 2 | 184792 | 167065 | 133422 | |
| 22 | PANX2 | pannexin 2 | 073150 | 073146 | 170638 | 23, 6, 37 |
| 22 | PVALB | parvalbumin | 100362 | 100360 | 100365 | 29, 17, 14 |
| 22 | PARVB | parvin beta | 188677 | 100347 | 138964 | 43, 28, 9 |
| 22 | PARVG | parvin gamma | 138964 | 188677 | 138944 | 5, 36, 39 |
| 22 | PNPLA3 | patatin like phospholipase domain containing 3 | 100344 | 100341 | 100347 | 35, 5, 16 |
| 22 | PNPLA5 | patatin like phospholipase domain containing 5 | 100341 | 130540 | 100344 | 35, 8, 12 |
| 22 | PPIL2 | peptidylprolyl isomerase like 2 | 100023 | 128228 | 100027 | 43, 34, 30 |
| 22 | PEX26 | peroxisomal biogenesis factor 26 | 215193 | 243156 | 183785 | 37, 24, 6 |
| 22 | PPARA | peroxisome proliferator activated receptor alpha | 186951 | 182257 | 205643 | 25, 2, 19 |
| 22 | PES1 | pescadillo ribosomal biogenesis factor 1 | 100029 | 128242 | 185339 | 36, 3, 21 |
| 22 | PHF21B | PHD finger protein 21B | 056487 | 241484 | 093000 | 14, 38, 8 |
| 22 | PHF5A | PHD finger protein 5A | 100410 | 183864 | 100412 | 26, 21, 35 |
| 22 | PI4KA | phosphatidylinositol 4-kinase alpha | 241973 | 099917 | 099937 | |
| 22 | PITPNB | phosphatidylinositol transfer protein beta | 180957 | 169184 | 100154 | 13, 9, 40 |
| 22 | PISD | phosphatidylserine decarboxylase | 241878 | 198089 | 183530 | 31, 32, 9 |
| 22 | PIK3IP1 | phosphoinositide-3-kinase interacting protein 1 | 100100 | 182541 | 100105 | 36, 39, 29 |
| 22 | PLA2G3 | phospholipase A2 group III | 100078 | 185133 | 138942 | 3, 2, 16 |
| 22 | PLA2G6 | phospholipase A2 group VI | 184381 | 128298 | 185022 | 39, 38, 42 |
| 22 | PMM1 | phosphomannomutase 1 | 100417 | 172346 | 100418 | 1, 35, 3 |
| 22 | PIM3 | Pim-3 proto-oncogene, serine/threonine kinase | 198355 | 184164 | 188263 | 43, 28, 21 |
| 22 | PIWIL3 | piwi like RNA-mediated gene silencing 3 | 184571 | 178026 | 167037 | 38, |
| 22 | PDGFB | platelet derived growth factor subunit B | 100311 | 100307 | 100316 | 24, 45, 32 |
| 22 | PLXNB2 | plexin B2 | 196576 | 185386 | 205593 | 26, 27, 22 |
| 22 | PKDREJ | polycystin (PKD) family receptor for egg jelly | 130943 | 205643 | 075234 | 24, 28, 38 |
| 22 | KCTD17 | potassium channel tetramerization domain containing 17 | 100379 | 128309 | 187045 | 31, 27, 22 |
| 22 | KCNJ4 | potassium voltage-gated channel subfamily J member 4 | 168135 | 213923 | 100196 | 15, 8, 20 |
| 22 | POTEH | POTE ankyrin domain family member H | 198062 | 130538 | 198445 | 38, |
| 22 | PATZ1 | POZ/BTB and AT hook containing zinc finger 1 | 100105 | 100100 | 185721 | |
| 22 | VPREB1 | pre-B lymphocyte 1 | 169575 | 211642 | 211643 | 5, 38, |
| 22 | VPREB3 | pre-B lymphocyte 3 | 128218 | 187792 | 169314 | 41, 36, 22 |
| 22 | PRAME | preferentially expressed antigen in melanoma | 185686 | 169548 | 211656 | 7, 35, 9 |
| 22 | PRODH | proline dehydrogenase 1 | 100033 | 183628 | 279560 | 12, 24, 15 |
| 22 | PRR14L | proline rich 14 like | 183530 | 241878 | 100150 | 7, 23, 1 |
| 22 | PRR34 | proline rich 34 | 182257 | 188064 | 186951 | |
| 22 | PRR5 | proline rich 5 | 186654 | 188636 | 248405 | 32, 5, 29 |
| 22 | PICK1 | protein interacting with PRKCA1 | 100151 | 100146 | 100156 | 36, 4, 35 |
| 22 | PACSIN2 | protein kinase C and casein kinase substrate in neurons 2 | 100266 | 242247 | 100271 | 3, 24, 5 |
| 22 | PPP6R2 | protein phosphatase 6 regulatory subunit 2 | 100239 | 205593 | 100241 | 3, 41, 34 |
| 22 | PPM1F | protein phosphatase, Mg2+/Mn2+ dependent 1F | 100034 | 100030 | 100038 | 43, 7, 1 |
| 22 | PRR5-ARHGAP8 | PRR5-ARHGAP8 readthrough | 248405 | 186654 | 241484 | |
| 22 | P2RX6 | purinergic receptor P2X 6 | 099957 | 184436 | 099960 | 36, 25, 14 |
| 22 | PDXP | pyridoxal phosphatase | 241360 | 100092 | 100097 | 5, 15, 3 |
| 22 | RABL2B | RAB, member of RAS oncogene family-like 2B | 079974 | 100312 | 215012 | 15, 11, 41 |
| 22 | RAB36 | RAB36, member RAS oncogene family | 100228 | 128266 | 186716 | 3, 29, 28 |
| 22 | RSPH14 | radial spoke head 14 homolog | 100218 | 211685 | 128266 | 2, 30, 38 |
| 22 | RGL4 | ral guanine nucleotide dissociation stimulator like 4 | 159496 | 189269 | 187792 | 5, 13, 36 |
| 22 | RANBP1 | RAN binding protein 1 | 099901 | 099899 | 099904 | 43, 22, 6 |
| 22 | RANGAP1 | Ran GTPase activating protein 1 | 100401 | 100399 | 100403 | 20, 38, |
| 22 | RASL10A | RAS like family 10 member A | 100276 | 185340 | 100280 | 5, 8, 27 |
| 22 | RASD2 | RASD family member 2 | 100302 | 100297 | 198125 | 33, 2, 27 |
| 22 | RAC2 | ras-related C3 botulinum toxin substrate 2 (rho family, small GTP binding protein | 128340 | 278195 | 100055 | 41, 21, 5 |
| | | Rac2) | | | | |
| 22 | RFPL1 | ret finger protein like 1 | 128250 | 100280 | 100285 | |
| 22 | RFPL2 | ret finger protein like 2 | 128253 | 205856 | 279219 | 25, 23, 33 |
| 22 | RFPL3 | ret finger protein like 3 | 128276 | 100191 | 205853 | 38, 8, |
| 22 | RTN4R | reticulon 4 receptor | 040608 | 234409 | 128185 | 9, 27, 5 |
| 22 | RFPL3S | RFPL3 antisense | 205853 | 128276 | 100220 | 38, 10, 39 |
| 22 | ARHGAP8 | Rho GTPase activating protein 8 | 241484 | 248405 | 056487 | |
| 22 | RHBDD3 | rhomboid domain containing 3 | 100263 | 186998 | 182944 | 5, 6, 33 |
| 22 | RIBC2 | RIB43A domain with coiled-coils 2 | 128408 | 077935 | 077942 | 9, 31, 20 |
| 22 | RPL3 | ribosomal protein L3 | 100316 | 100311 | 100321 | 41, 21, 30 |
| 22 | RPS19BP1 | ribosomal protein S19 binding protein 1 | 187051 | 128272 | 100346 | 34, 41, 37 |
| 22 | RRP7A | ribosomal RNA processing 7 homolog A | 189306 | 235568 | 183569 | 21, 29, 41 |
| 22 | RIMBP3 | RIMS binding protein 3 | 275793 | 278558 | 183628 | 38, 20, 5 |
| 22 | RIMBP3B | RIMS binding protein 3B | 274600 | 133475 | 169635 | 38, 27, 14 |
| 22 | RIMBP3C | RIMS binding protein 30 | 183246 | 206140 | 185651 | 38, 27, 14 |
| 22 | RNF185 | ring finger protein 185 | 138942 | 100078 | 182541 | 39, 29, 42 |
| 22 | RNF215 | ring finger protein 215 | 099999 | 187860 | 100003 | |
| 22 | RBX1 | ring-box 1 | 100387 | 172404 | 100393 | 10, 34, 15 |
| 22 | RTCB | RNA 2′,3′-cydic phosphate and 5′-OH ligase | 100220 | 205853 | 184459 | 34, 2, 30 |
| 22 | RBFOX2 | RNA binding protein, fox-1 homolog 2 | 100320 | 128313 | 128284 | 29, 12, 15 |
| 22 | POLR2F | RNA polymerase II subunit F | 100142 | 128346 | 100146 | 43, 15, 8 |
| 22 | POLR3H | RNA polymerase III subunit H | 100413 | 100412 | 172346 | 43, 40, 35 |
| 22 | SUN2 | Sad1 and UNC84 domain containing 2 | 100242 | 100226 | 100246 | 1, 39, 22 |
| 22 | SAMM50 | SAMM50 sorting and assembly machinery component | 100347 | 100344 | 188677 | 1, 41, 21 |
| 22 | SCARF2 | scavenger receptor class F member 2 | 244486 | 185252 | 277971 | 6, 27, 14 |
| 22 | SCO2 | SCO2, cytochrome c oxidase assembly protein | 130489 | 025770 | 025708 | |
| 22 | SEC14L2 | SEC14 like lipid binding 2 | 100003 | 099999 | 249590 | 33, 8, 43 |
| 22 | SEC14L3 | SEC14 like lipid binding 3 | 100012 | 242114 | 133488 | 42, 20, 30 |
| 22 | SEC14L4 | SEC14 like lipid binding 4 | 133488 | 100012 | 214491 | 15, 7, 36 |
| 22 | SEC14L6 | SEC14 like lipid binding 6 | 214491 | 133488 | 128242 | 17, 12, 8 |
| 22 | SEZ6L | seizure related 6 homolog like | 100095 | 133454 | 128203 | 26, 28, 20 |
| 22 | SELENOM | selenoprotein M | 198832 | 183963 | 185133 | |
| 22 | SELENOO | selenoprotein O | 073169 | 170638 | 128159 | 9, 21, 5 |
| 22 | SEPT3 | septin 3 | 100167 | 100162 | 183066 | 26, 8, 45 |
| 22 | SEPT5 | septin 5 | 184702 | 184113 | 203618 | |
| 22 | SERHL2 | serine hydrolase-like 2 | 183569 | 189306 | 100227 | 29, 12, 33 |
| 22 | SERPIND1 | serpin family D member 1 | 099937 | 241973 | 099940 | 19, 45, 23 |
| 22 | SBF1 | SET binding factor 1 | 100241 | 100239 | 128165 | 22, 21, 9 |
| 22 | SFI1 | SFI1 centrin binding protein | 198089 | 184708 | 241878 | 26, 39, 40 |
| 22 | SHANK3 | SH3 and multiple ankyrin repeat domains 3 | 251322 | 100299 | 100312 | 6, 26, 36 |
| 22 | SH3BP1 | SH3 domain binding protein 1 | 100092 | 100083 | 241360 | 1, 4, 41 |
| 22 | SHISA8 | shisa family member 8 | 234965 | 198911 | 159958 | 22, 16, |
| 22 | SCUBE1 | signal peptide, CUB domain and EGF like domain containing 1 | 159307 | 100304 | 186732 | 26, 10, 14 |
| 22 | SMDT1 | single-pass membrane protein with aspartate rich tail 1 | 183172 | 177096 | 184983 | 31, 41, 14 |
| 22 | SGSM1 | small G protein signaling modulator 1 | 167037 | 184571 | 206069 | 29, 20, 14 |
| 22 | SGSM3 | small G protein signaling modulator 3 | 100359 | 239900 | 196588 | 13, 5, 31 |
| 22 | SNRPD3 | small nuclear ribonucleoprotein D3 polypeptide | 100028 | 138867 | 100031 | 29, 21, 26 |
| 22 | SMTN | smoothelin | 183963 | 133422 | 198832 | 44, 13, 42 |
| 22 | SNU13 | SNU13 homolog, small nuclear ribonucleoprotein (U4/U6.U5) | 100138 | 196419 | 184208 | |
| 22 | SLC16A8 | solute carrier family 16 member 8 | 100156 | 100151 | 128298 | 14, 9, 33 |
| 22 | SLC2A11 | solute carrier family 2 member 11 | 133460 | 099958 | 251357 | |
| 22 | SLC25A1 | solute carrier family 25 member 1 | 100075 | 063515 | 070371 | 34, 1, 3 |
| 22 | SLC25A17 | solute carrier family 25 member 17 | 100372 | 128285 | 100380 | 2, 35, 7 |
| 22 | SLC25A18 | solute carrier family 25 member 18 | 182902 | 099954 | 131100 | 19, 8, 6 |
| 22 | SLC35E4 | solute carrier family 35 member E4 | 100036 | 185339 | 167065 | 38, 6, 3 |
| 22 | SLC5A1 | solute carrier family 5 member 1 | 100170 | 128245 | 205856 | 42, 15, 16 |
| 22 | SLC5A4 | solute carrier family 5 member 4 | 100191 | 279219 | 128276 | 35, |
| 22 | SLC7A4 | solute carrier family 7 member 4 | 099960 | 099957 | 187905 | 32, 38, 35 |
| 22 | SSTR3 | somatostatin receptor 3 | 278195 | 133466 | 128340 | 39, 22, 28 |
| 22 | SPECC1L- | SPECC1L-ADORA2A readthrough (NMD candidate) | 258555 | 100014 | 128271 | |
| | ADORA2A | | | | | |
| 22 | SPECC1L | sperm antigen with calponin homology and coiled-coil domains 1 like | 100014 | 099998 | 258555 | 41, 22, 33 |
| 22 | SF3A1 | splicing factor 3a subunit 1 | 099995 | 099992 | 187860 | 39, 22, 41 |
| 22 | SRRD | SRR1 domain containing | 100104 | 100099 | 100109 | 27, 7, 35 |
| 22 | SOX10 | SRY-box 10 | 100146 | 100142 | 100151 | 35, 26, 11 |
| 22 | SREBF2 | sterol regulatory element binding transcription factor 2 | 198911 | 100147 | 234965 | 30, 21, 22 |
| 22 | SDF2L1 | stromal cell derived factor 2 like 1 | 128228 | 161180 | 100023 | 34, 18, 28 |
| 22 | SMC1B | structural maintenance of chromosomes 1B | 077935 | 100376 | 128408 | 10, 22, 5 |
| 22 | SULT4A1 | sulfotransferase family 4A member 1 | 130540 | 186976 | 100341 | 26, 8, 14 |
| 22 | ST13 | suppression of tumorigenicity 13 (colon carcinoma) (Hsp70 interacting protein) | 100380 | 100372 | 196236 | 31, 40, 45 |
| 22 | SUSD2 | sushi domain containing 2 | 099994 | 099991 | 099998 | 17, 42, 20 |
| 22 | SMARCB1 | SWI/SNF related, matrix associated, actin dependent regulator of chromatin, | 099956 | 280178 | 099958 | 21, 18, 41 |
| | | subfamily b, member 1 | | | | |
| 22 | SYN3 | synapsin III | 185666 | 100225 | 100234 | |
| 22 | SYNGR1 | synaptogyrin 1 | 100321 | 100316 | 100324 | 14, 8, 35 |
| 22 | SYCE3 | synaptonemal complex central element protein 3 | 217442 | 130487 | 205560 | 38, 17, 20 |
| 22 | SNAP29 | synaptosome associated protein 29 | 099940 | 099937 | 099942 | 9, 44, 23 |
| 22 | TOM1 | target of myb1 membrane trafficking protein | 100284 | 100281 | 100292 | 36, 5, 39 |
| 22 | TBC1D10A | TBC1 domain family member 10A | 099992 | 248751 | 099995 | 32, 5, 36 |
| 22 | TBC1D22A | TBC1 domain family member 22A | 054611 | 100422 | 280080 | 18, 3, 13 |
| 22 | TBX1 | T-box 1 | 184058 | 203618 | 185838 | 20, 24, 27 |
| 22 | TEF | TEF, PAR bZIP transcription factor | 167074 | 100403 | 183864 | 27, 8, 11 |
| 22 | TEX33 | testis expressed 33 | 185264 | 100368 | 128311 | 38, 8, |
| 22 | TSSK2 | testis specific serine kinase 2 | 206203 | 100056 | 063515 | 38, 15, 8 |
| 22 | TTC28 | tetratricopeptide repeat domain 28 | 100154 | 180957 | 183765 | 11, 36, 18 |
| 22 | TTC38 | tetratricopeptide repeat domain 38 | 075234 | 130943 | 075218 | 34, 17, 33 |
| 22 | TAB1 | TGF-beta activated kinase 1 (MAP3K7) binding protein 1 | 100324 | 100321 | 128268 | 41, 18, 43 |
| 22 | THAP7 | THAP domain containing 7 | 184436 | 099949 | 099957 | 29, 31, 2 |
| 22 | TXN2 | thioredoxin 2 | 100348 | 100345 | 100350 | 34, 30, 7 |
| 22 | TXNRD2 | thioredoxin reductase 2 | 184470 | 215012 | 093010 | 9, 33, 41 |
| 22 | TST | thiosulfate sulfurtransferase | 128311 | 185264 | 128309 | 3, 1, 16 |
| 22 | THOC5 | THO complex 5 | 100296 | 100285 | 184117 | 3, 41, 45 |
| 22 | TYMP | thymidine phosphorylase | 025708 | 130489 | 177989 | 32, 5, 18 |
| 22 | TIMP3 | TIMP metallopeptidase inhibitor 3 | 100234 | 185666 | 133424 | 26, 11, 32 |
| 22 | TNFRSF13C | TNF receptor superfamily member 130 | 159958 | 234965 | 100162 | 22, |
| 22 | TOP3B | topoisomerase (DNA) III beta | 100038 | 100034 | 211637 | 39, 13, 24 |
| 22 | TRABD | TraB domain containing | 170638 | 073150 | 073169 | 21, 22, 34 |
| 22 | TCN2 | transcobalamin 2 | 185339 | 100029 | 100036 | 7, 24, 2 |
| 22 | TCF20 | transcription factor 20 | 100207 | 205702 | 235568 | 18, 4, 16 |
| 22 | TOB2 | transducer of ERBB2, 2 | 183864 | 167074 | 100410 | 9, 1, 22 |
| 22 | TOMM22 | translocase of outer mitochondrial membrane 22 | 100216 | 100211 | 100221 | 27, 7, 37 |
| 22 | TSPO | translocator protein | 100300 | 100294 | 100304 | 3, 6, 34 |
| 22 | TMPRSS6 | transmembrane protease, serine 6 | 187045 | 100379 | 100385 | 2, 19, 38 |
| 22 | TMEM184B | transmembrane protein 184B | 198792 | 185022 | 213923 | 32, 27, 42 |
| 22 | TMEM191B | transmembrane protein 191B | 278558 | 274252 | 275793 | |
| 22 | TMEM191C | transmembrane protein 191C | 206140 | 169635 | 183246 | 29, 3, 38 |
| 22 | TMEM211 | transmembrane protein 211 | 206069 | 167037 | 197077 | 10, 44, |
| 22 | TANGO2 | transport and golgi organization 2 homolog | 183597 | 099889 | 128191 | 40, 5, 26 |
| 22 | TNRC6B | trinucleotide repeat containing 6B | 100354 | 133477 | 239900 | 26, 31, 22 |
| 22 | TRIOBP | TRIO and F-actin binding protein | 100106 | 100101 | 189060 | 34, 41, 35 |
| 22 | TRMU | tRNA 5-methylaminomethyl-2-thiouridylate methyltransferase | 100416 | 075218 | 075275 | 3, 9, 39 |
| 22 | TRMT2A | tRNA methyltransferase 2 homolog A | 099899 | 128191 | 099901 | 36, 2, 9 |
| 22 | TUBA8 | tubulin alpha 8 | 183785 | 215193 | 184979 | 25, 15, 8 |
| 22 | TUBGCP6 | tubulin gamma complex associated protein 6 | 128159 | 073169 | 100429 | 26, 33, 39 |
| 22 | TTLL1 | tubulin tyrosine ligase like 1 | 100271 | 100266 | 100290 | 34, 8, 6 |
| 22 | TTLL12 | tubulin tyrosine ligase like 12 | 100304 | 100300 | 159307 | 21, 3, 9 |
| 22 | TTLL8 | tubulin tyrosine ligase like 8 | 138892 | 188263 | 100427 | |
| 22 | TFIP11 | tuftelin interacting protein 11 | 100109 | 100104 | 128294 | 24, 38, 3 |
| 22 | YWHAH | tyrosine 3-monooxygenase/tryptophan 5-monooxygenase activation protein eta | 128245 | 100150 | 100170 | 28, 5, 8 |
| 22 | TPST2 | tyrosylprotein sulfotransferase 2 | 128294 | 100109 | 100122 | 32, 5, 40 |
| 22 | UQCR10 | ubiquinol-cytochrome c reductase, complex III subunit X | 184076 | 100319 | 100325 | 41, 34, 25 |
| 22 | UBE2L3 | ubiquitin conjugating enzyme E2 L3 | 185651 | 183246 | 161179 | 29, 2, 39 |
| 22 | UFD1L | ubiquitin fusion degradation 1 like (yeast) | 070010 | 185608 | 093009 | 43, 29, 30 |
| 22 | USP18 | ubiquitin specific peptidase 18 | 184979 | 183785 | 274252 | 4, 18, 45 |
| 22 | USP41 | ubiquitin specific peptidase 41 | 161133 | 128185 | 185252 | |
| 22 | FP325331.1 | Uncharacterized protein UNQ6126/PRO20091 | 278881 | 280080 | 219438 | |
| 22 | UPK3A | uroplakin 3A | 100373 | 100364 | 100376 | 33, 14, |
| 22 | WBP2NL | WBP2 N-terminal like | 183066 | 100167 | 198951 | |
| 22 | WNT7B | Wnt family member 7B | 188064 | 130638 | 182257 | 34, 33, 14 |
| 22 | XBP1 | X-box binding protein 1 | 100219 | 159873 | 183579 | 11, 23, 28 |
| 22 | XKR3 | XK related 3 | 172967 | 198445 | 215568 | 38, 8, |
| 22 | XPNPEP3 | X-prolyl aminopeptidase 3 | 196236 | 100380 | 172404 | 35, 6, 15 |
| 22 | XRCC6 | X-ray repair cross complementing 6 | 196419 | 100418 | 100138 | 13, 45, 35 |
| 22 | YDJC | YdjC homolog (bacterial) | 161179 | 185651 | 161180 | 41, 5, 9 |
| 22 | YPEL1 | yippee like 1 | 100027 | 100023 | 100030 | 43, 38, 21 |
| 22 | ZNRF3 | zinc and ring finger 3 | 183579 | 100219 | 100249 | 25, 12, 6 |
| 22 | ZBED4 | zinc finger BED-type containing 4 | 100426 | 100425 | 182858 | 22, 1, 37 |
| 22 | ZC3H7B | zinc finger COCH-type containing 7B | 100403 | 100401 | 167074 | 24, 21, 42 |
| 22 | ZDHHC8 | zinc finger DHHC-type containing 8 | 099904 | 099901 | 234409 | 27, 6, 26 |
| 22 | ZMAT5 | zinc finger matrin-type 5 | 100319 | 100314 | 184076 | 15, 39, 41 |
| 22 | ZNF280A | zinc finger protein 280A | 169548 | 275004 | 185686 | 7, 14, 38 |
| 22 | ZNF280B | zinc finger protein 280B | 275004 | 211655 | 169548 | 29, 11, 38 |
| 22 | ZNF70 | zinc finger protein 70 | 187792 | 159496 | 128218 | 24, 44, 7 |
| 22 | ZNF74 | zinc finger protein 74 | 185252 | 161133 | 244486 | 12, 1, 31 |
| MT | MT-ATP6 | mitochondrially encoded ATP synthase 6 | 198899 | 228253 | 198938 | |
| MT | MT-ATP8 | mitochondrially encoded ATP synthase 8 | 228253 | 198712 | 198899 | |
| MT | MT-CYB | mitochondrially encoded cytochrome b | 198727 | 198695 | 198840 | |
| MT | MT-CO1 | mitochondrially encoded cytochrome c oxidase I | 198804 | 198763 | 198712 | |
| MT | MT-CO2 | mitochondrially encoded cytochrome c oxidase II | 198712 | 198804 | 228253 | |
| MT | MT-CO3 | mitochondrially encoded cytochrome c oxidase III | 198938 | 198899 | 198840 | |
| MT | MT-ND1 | mitochondrially encoded NADH:ubiquinone oxidoreductase core subunit 1 | 198888 | 198840 | 198763 | |
| MT | MT-ND2 | mitochondrially encoded NADH:ubiquinone oxidoreductase core subunit 2 | 198763 | 198888 | 198804 | |
| MT | MT-ND3 | mitochondrially encoded NADH:ubiquinone oxidoreductase core subunit 3 | 198840 | 198938 | 212907 | |
| MT | MT-ND4 | mitochondrially encoded NADH:ubiquinone oxidoreductase core subunit 4 | 198886 | 212907 | 198786 | |
| MT | MT-ND4L | mitochondrially encoded NADH:ubiquinone oxidoreductase core subunit 4L | 212907 | 198840 | 198886 | |
| MT | MT-ND5 | mitochondrially encoded NADH:ubiquinone oxidoreductase core subunit 5 | 198786 | 198886 | 198695 | |
| MT | MT-ND6 | mitochondrially encoded NADH:ubiquinone oxidoreductase core subunit 6 | 198695 | 198786 | 198727 | |
| X | ALAS2 | 5′-aminolevulinate synthase 2 | 158578 | 169188 | 238269 | 43, 5, 19 |
| X | HTR2C | 5-hydroxytryptamine receptor 2C | 147246 | 126016 | 123496 | 8, 25, 12 |
| X | PFKFB1 | 6-phosphofructo-2-kinase/fructose-2,6-biphosphatase 1 | 158571 | 067445 | 169188 | 25, 19, 39 |
| X | ASMT | acetylserotonin O-methyltransferase | 196433 | 197976 | 169084 | 29, 22, 16 |
| X | ASMTL | acetylserotonin O-methyltransferase-like | 169093 | 169100 | 182162 | 41, 3, 39 |
| X | ACRC | acidic repeat containing | 147174 | 147162 | 186810 | 26, 38, 5 |
| X | ACTRT1 | actin related protein T1 | 123165 | 183631 | 102038 | 38, 19, |
| X | ACSL4 | acyl-CoA synthetase long-chain family member 4 | 068366 | 176076 | 157600 | |
| X | ACOT9 | acyl-CoA thioesterase 9 | 123130 | 123131 | 130066 | 29, 4, 40 |
| X | AWAT1 | acyl-CoA wax alcohol acyltransferase 1 | 204195 | 184210 | 186912 | 15, 35, |
| X | AWAT2 | acyl-CoA wax alcohol acyltransferase 2 | 147160 | 158813 | 189401 | 35, 38, 8 |
| X | AP1S2 | adaptor related protein complex 1 sigma 2 subunit | 182287 | 169249 | 126010 | 35, 15, 29 |
| X | ADGRG2 | adhesion G protein-coupled receptor G2 | 173698 | 044446 | 131828 | 29, 4, 6 |
| X | ADGRG4 | adhesion G protein-coupled receptor G4 | 156920 | 129680 | 102239 | 14, |
| X | ARL13A | ADP ribosylation factor like GTPase 13A | 174225 | 182489 | 188917 | 45, 38, 10 |
| X | AFF2 | AF4/FMR2 family member 2 | 155966 | 176988 | 010404 | 22, 5, 24 |
| X | AKAP14 | A-kinase anchoring protein 14 | 186471 | 125356 | 101882 | 10, 38, 20 |
| X | AKAP17A | A-kinase anchoring protein 17A | 197976 | 182162 | 196433 | 29, 22, 16 |
| X | AKAP4 | A-kinase anchoring protein 4 | 147081 | 171365 | 147082 | 38, |
| X | ALG13 | ALG13, UDP-N-acetylglucosaminyltransferase subunit | 101901 | 260802 | 072315 | |
| X | AMMECR1 | Alport syndrome, mental retardation, midface hypoplasia and elliptocytosis | 101935 | 157600 | 243978 | 18, 32, 45 |
| | | chromosomal region gene 1 | | | | |
| X | AMELX | amelogenin, X-linked | 125363 | 047648 | 005302 | 10, |
| X | AR | androgen receptor | 169083 | 131080 | 079482 | 33, 26, 11 |
| X | AMOT | angiomotin | 126016 | 182508 | 147246 | 24, 15, 17 |
| X | ACE2 | angiotensin I converting enzyme 2 | 130234 | 102010 | 147003 | 38, 16, 17 |
| X | AGTR2 | angiotensin II receptor type 2 | 180772 | 102024 | 268104 | 15, 17, 38 |
| X | ASB11 | ankyrin repeat and SOCS box containing 11 | 165192 | 102048 | 165195 | 25, |
| X | ASB12 | ankyrin repeat and SOCS box containing 12 | 198881 | 184675 | 102043 | 25, 15, 38 |
| X | ASB9 | ankyrin repeat and SOCS box containing 9 | 102048 | 130150 | 165192 | 28, 35, 38 |
| X | ANOS1 | anosmin 1 | 011201 | 205642 | 183304 | 29, 34, 38 |
| X | AMER1 | APC membrane recruitment protein 1 | 184675 | 131089 | 198881 | 36, 40, 15 |
| X | APLN | apelin | 171388 | 122126 | 122121 | 45, 43, 26 |
| X | APOO | apolipoprotein O | 184831 | 130066 | 165182 | 4, 10, 14 |
| X | APOOL | apolipoprotein O like | 155008 | 165259 | 184788 | 45, 4, 23 |
| X | AIFM1 | apoptosis inducing factor mitochondria associated 1 | 156709 | 102034 | 134594 | 17, 3, 35 |
| X | APEX2 | apurinic/apyrimidinic endodeoxyribonuclease 2 | 169188 | 158571 | 158578 | 9, 23, 27 |
| X | ARAF | A-Raf proto-oncogene, serine/threonine kinase | 078061 | 147124 | 008056 | 9, 36, 45 |
| X | AVPR2 | arginine vasopressin receptor 2 | 126895 | 198910 | 089820 | 28, 20, |
| X | ARX | aristaless related homeobox | 004848 | 101868 | 176774 | 27, 28, 6 |
| X | ARMCX1 | armadillo repeat containing, X-linked 1 | 126947 | 196440 | 198960 | 45, 2, 43 |
| X | ARMCX2 | armadillo repeat containing, X-linked 2 | 184867 | 102401 | 126952 | 11, 1, 12 |
| X | ARMCX3 | armadillo repeat containing, X-linked 3 | 102401 | 198960 | 184867 | 40, 43, 2 |
| X | ARMCX4 | armadillo repeat containing, X-linked 4 | 196440 | 126945 | 126947 | 31, 44, 8 |
| X | ARMCX5 | armadillo repeat containing, X-linked 5 | 125962 | 158164 | 198932 | 6, 18, 7 |
| X | ARMCX6 | armadillo repeat containing, X-linked 6 | 198960 | 126947 | 102401 | 36, 2, 35 |
| X | ARR3 | arrestin 3 | 120500 | 186912 | 147127 | 14, 25, |
| X | ARSD | arylsulfatase D | 006756 | 056998 | 157399 | 43, 17, 42 |
| X | ARSE | arylsulfatase E (chondrodysplasia punctata 1) | 157399 | 006756 | 205667 | 17, 43, 26 |
| X | ARSF | arylsulfatase F | 062096 | 205667 | 101825 | 17, 33, 38 |
| X | ARSH | arylsulfatase family member H | 205667 | 157399 | 062096 | 28, |
| X | ATXN3L | ataxin 3 like | 123594 | 187268 | 198759 | 38, 8, |
| X | ABCB7 | ATP binding cassette subfamily B member 7 | 131269 | 050030 | 094841 | 22, 39, 36 |
| X | ABCD1 | ATP binding cassette subfamily D member 1 | 101986 | 185825 | 198753 | 9, 28, 32 |
| X | ATP7A | ATPase copper transporting alpha | 165240 | 131174 | 226784 | 24, 29, 45 |
| X | ATP6AP1 | ATPase H+ transporting accessory protein 1 | 071553 | 102125 | 203879 | 36, 2, 8 |
| X | ATP6AP2 | ATPase H+ transporting accessory protein 2 | 182220 | 183337 | 238205 | 29, 11, 8 |
| X | ATP1B4 | ATPase Na+/K+ transporting family member beta 4 | 101892 | 125355 | 005893 | 1, 24, 25 |
| X | ATP11C | ATPase phospholipid transporting 11C | 101974 | 101977 | 203933 | 1, 12, 4 |
| X | ATP2B3 | ATPase plasma membrane Ca2+ transporting 3 | 067842 | 182492 | 262919 | |
| X | ATRX | ATRX, chromatin remodeler | 085224 | 196468 | 102158 | 11, 29, 23 |
| X | ATG4A | autophagy related 4A cysteine peptidase | 101844 | 101843 | 197565 | 21, 36, 45 |
| X | BHLHB9 | basic helix-loop-helix domain containing, class B, 9 | 198908 | 158301 | 102128 | 42, 26, 8 |
| X | BCAP31 | B-cell receptor-associated protein 31 | 185825 | 130821 | 101986 | 30, 37, 29 |
| X | BCOR | BCL6 corepressor | 183337 | 165175 | 182220 | 22, 39, 25 |
| X | BCORL1 | BCL6 corepressor-like 1 | 085185 | 156697 | 102034 | 43, 32, 40 |
| X | BEND2 | BEN domain containing 2 | 177324 | 131831 | 102098 | 38, 25, |
| X | BGN | biglycan | 182492 | 213397 | 067842 | 36, 10, 45 |
| X | BMX | BMX non-receptor tyrosine kinase | 102010 | 087842 | 130234 | 45, 40, 43 |
| X | BRS3 | bombesin receptor subtype 3 | 102239 | 156920 | 102241 | 17, 38, |
| X | BMP15 | bone morphogenetic protein 15 | 130385 | 158352 | 122824 | |
| X | BEX1 | brain expressed X-linked 1 | 133169 | 102128 | 147206 | 31, 29, 28 |
| X | BEX2 | brain expressed X-linked 2 | 133134 | 204065 | 182916 | 31, 28, 8 |
| X | BEX3 | brain expressed X-linked 3 | 166681 | 185222 | 172476 | |
| X | BEX4 | brain expressed X-linked 4 | 102409 | 147206 | 180964 | 26, 1, 34 |
| X | BEX5 | brain expressed X-linked 5 | 184515 | 204071 | 269405 | 44, 8, 17 |
| X | BRCC3 | BRCA1/BRCA2-containing complex subunit 3 | 185515 | 214827 | 155959 | 25, 30, 9 |
| X | BRWD3 | bromodomain and WD repeat domain containing 3 | 165288 | 174016 | 198157 | 1, 3, 31 |
| X | BTK | Bruton tyrosine kinase | 010671 | 126953 | 241343 | 21, 41, 5 |
| X | C1GALT1C1 | C1GALT1 specific chaperone 1 | 171155 | 232119 | 236446 | 31, 29, 30 |
| X | CACNA1F | calcium voltage-gated channel subunit alpha1 F | 102001 | 102003 | 101997 | 14, 25, 39 |
| X | CASK | calcium/calmodulin dependent serine protein kinase | 147044 | 188937 | 171659 | 29, 12, 4 |
| X | CAPN6 | calpain 6 | 077274 | 077264 | 077279 | 32, 25, 42 |
| X | CTAG1A | cancer/testis antigen 1A | 268651 | 269335 | 184033 | |
| X | CTAG1B | cancer/testis antigen 1B | 184033 | 268651 | 126890 | |
| X | CTAG2 | cancer/testis antigen 2 | 126890 | 184033 | 160219 | 23, 3, 44 |
| X | CT55 | cancer/testis antigen 55 | 169551 | 224107 | 186376 | |
| X | CT83 | cancer/testis antigen 83 | 204019 | 268104 | 003096 | |
| X | CT45A1 | cancer/testis antigen family 45 member A1 | 268940 | 165359 | 269096 | 38, 20, |
| X | CT45A10 | cancer/testis antigen family 45 member A10 | 269586 | 270946 | 181433 | |
| X | CT45A2 | cancer/testis antigen family 45 member A2 | 271449 | 278289 | 273696 | |
| X | CT45A3 | cancer/testis antigen family 45 member A3 | 269096 | 268940 | 228836 | 37, 38, 35 |
| X | CT45A5 | cancer/testis antigen family 45 member A5 | 228836 | 269096 | 278289 | 34, 13, 6 |
| X | CT45A6 | cancer/testis antigen family 45 member A6 | 278289 | 228836 | 271449 | 19, 38, |
| X | CT45A7 | cancer/testis antigen family 45 member A7 | 273696 | 271449 | 278085 | |
| X | CT45A8 | cancer/testis antigen family 45 member A8 | 278085 | 273696 | 270946 | |
| X | CT45A9 | cancer/testis antigen family 45 member A9 | 270946 | 278085 | 269586 | |
| X | CT47A1 | cancer/testis antigen family 47, member A1 | 236371 | 242362 | 182890 | |
| X | CT47A10 | cancer/testis antigen family 47, member A10 | 224089 | 226929 | 226600 | 38, |
| X | CT47A11 | cancer/testis antigen family 47, member A11 | 226929 | 226685 | 224089 | 38, |
| X | CT47A12 | cancer/testis antigen family 47, member A12 | 226685 | 278646 | 226929 | |
| X | CT47A2 | cancer/testis antigen family 47, member A2 | 242362 | 236126 | 236371 | 38, |
| X | CT47A3 | cancer/testis antigen family 47, member A3 | 236126 | 230594 | 242362 | 38, |
| X | CT47A4 | cancer/testis antigen family 47, member A4 | 230594 | 237957 | 236126 | 38, |
| X | CT47A5 | cancer/testis antigen family 47, member A5 | 237957 | 226023 | 230594 | 38, |
| X | CT47A6 | cancer/testis antigen family 47, member A6 | 226023 | 228517 | 237957 | |
| X | CT47A7 | cancer/testis antigen family 47, member A7 | 228517 | 230347 | 226023 | |
| X | CT47A8 | cancer/testis antigen family 47, member A8 | 230347 | 226600 | 228517 | 38, |
| X | CT47A9 | cancer/testis antigen family 47, member A9 | 226600 | 224089 | 230347 | 38, |
| X | CT47B1 | cancer/testis antigen family 47, member B1 | 236446 | 171155 | 278646 | 26, 38, |
| X | CHST7 | carbohydrate sulfotransferase 7 | 147119 | 251192 | 065923 | 45, 28, 5 |
| X | CA5B | carbonic anhydrase 5B | 169239 | 147003 | 169249 | 11, 31, 13 |
| X | CDX4 | caudal type homeobox 4 | 131264 | 186462 | 204116 | |
| X | CITED1 | Cbp/p300 interacting transactivator with Glu/Asp rich carboxy-terminal domain 1 | 125931 | 198034 | 147099 | 31, 35, 38 |
| X | CD40LG | CD40 ligand | 102245 | 102243 | 129675 | 5, 39, |
| X | CD99 | CD99 molecule | 002586 | 214717 | 124343 | 28, |
| X | CD99L2 | CD99 molecule like 2 | 102181 | 063601 | 029993 | 8, 43, 42 |
| X | ARHGEF9 | Cdc42 guanine nucleotide exchange factor 9 | 131089 | 186767 | 184675 | 14, 2, 8 |
| X | CETN2 | centrin 2 | 147400 | 197172 | 147383 | 30, 11, 4 |
| X | CENPI | centromere protein I | 102384 | 126950 | 102385 | |
| X | CENPVL1 | Centromere protein V like 1 | 223591 | 283093 | 189369 | |
| X | CENPVL2 | Centromere protein V like 2 | 283093 | 224109 | 223591 | |
| X | CENPVL3 | Centromere protein V like 3 | 224109 | 196368 | 283093 | |
| X | CDR1 | cerebellar degeneration related protein 1 | 184258 | 134595 | 227234 | 8, 1, 30 |
| X | CLIC2 | chloride intracellular channel 2 | 155962 | 155961 | 277858 | 29, 34, 30 |
| X | CLCN4 | chloride voltage-gated channel 4 | 073464 | 047644 | 101871 | 8, 22, 14 |
| X | CLCN5 | chloride voltage-gated channel 5 | 171365 | 273820 | 147081 | 11, 17, 31 |
| X | CHM | CHM, Rab escort protein 1 | 188419 | 124429 | 126733 | 42, 45, 24 |
| X | CSAG1 | chondrosarcoma associated gene 1 | 198930 | 183305 | 213401 | 34, 10, 35 |
| X | CHRDL1 | chordin like 1 | 101938 | 243978 | 077264 | 29, 1, 44 |
| X | CXorf21 | chromosome X open reading frame 21 | 120280 | 169297 | 198814 | 36, 30, 22 |
| X | CXorf23 | chromosome X open reading frame 23 | 173681 | 147010 | 184368 | |
| X | CXorf36 | chromosome X open reading frame 36 | 147113 | 147050 | 147121 | 15, 1, 43 |
| X | CXorf38 | chromosome X open reading frame 38 | 185753 | 238205 | 180182 | 1, 6, 31 |
| X | CXorf40A | chromosome X open reading frame 40A | 197620 | 241489 | 283697 | 29, 21, 3 |
| X | CXorf40B | chromosome X open reading frame 40B | 197021 | 156009 | 283463 | 26, 3, 15 |
| X | CXorf49 | chromosome X open reading frame 49 | 215115 | 283599 | 215113 | |
| X | CXorf49B | chromosome X open reading frame 49B | 215113 | 215115 | 204131 | |
| X | CXorf51A | Chromosome X open reading frame 51A | 224440 | 235699 | 102081 | 38, |
| X | CXorf51B | Chromosome X open reading frame 51B | 235699 | 221870 | 224440 | |
| X | CXorf56 | chromosome X open reading frame 56 | 018610 | 005022 | 077721 | 34, 41, 43 |
| X | CXorf57 | chromosome X open reading frame 57 | 147231 | 157502 | 133135 | 4, 38, 28 |
| X | CXorf58 | chromosome X open reading frame 58 | 165182 | 184831 | 174010 | 28, 19, 38 |
| X | CXorf65 | chromosome X open reading frame 65 | 204165 | 184481 | 147168 | 38, 7, |
| X | CXorf66 | chromosome X open reading frame 66 | 203933 | 101974 | 134595 | 38, |
| X | CXorf67 | chromosome X open reading frame 67 | 187690 | 122824 | 196368 | 32, 35, 38 |
| X | CFAP47 | cilia and flagella associated protein 47 | 165164 | 189023 | 198173 | 42, 30, 23 |
| X | CLDN2 | daudin 2 | 165376 | 147223 | 089682 | 17, 37, 16 |
| X | CLDN34 | daudin 34 | 234469 | 146950 | 047644 | |
| X | CSTF2 | cleavage stimulation factor subunit 2 | 101811 | 102362 | 007952 | 7, 35, 25 |
| X | F9 | coagulation factor IX | 101981 | 283147 | 101977 | 19, |
| X | F8 | coagulation factor VIII | 185010 | 203870 | 274183 | 29, 26, 31 |
| X | F8A1 | coagulation factor VIII-associated 1 | 277203 | 274183 | 165775 | 26, 9, 28 |
| X | F8A2 | coagulation factor VIII-associated 2 | 274791 | 277858 | 277150 | 40, 22, 44 |
| X | F8A3 | coagulation factor VIII-associated 3 | 277150 | 274791 | 277745 | |
| X | CCDC120 | coiled-coil domain containing 120 | 147144 | 068323 | 243279 | 24, 42, 4 |
| X | CCDC160 | coiled-coil domain containing 160 | 203952 | 147257 | 156531 | 12, 17, 20 |
| X | CCDC22 | coiled-coil domain containing 22 | 101997 | 102001 | 049768 | 21, 5, 7 |
| X | COL4A5 | collagen type IV alpha 5 chain | 188153 | 197565 | 260548 | 11, 12, 31 |
| X | COL4A6 | collagen type IV alpha 6 chain | 197565 | 101844 | 188153 | 12, 9, 13 |
| X | CSF2RA | colony stimulating factor 2 receptor alpha subunit | 198223 | 205755 | 185291 | 32, 5, 7 |
| X | CFP | complement factor properdin | 126759 | 102265 | 126767 | 36, 41, 1 |
| X | CNKSR2 | connector enhancer of kinase suppressor of Ras 2 | 149970 | 177189 | 185915 | 8, 26, 11 |
| X | CPXCR1 | CPX chromosome region, candidate 1 | 147183 | 102271 | 153779 | 38, 35, |
| X | CTPS2 | CTP synthase 2 | 047230 | 182798 | 169906 | 22, 18, 25 |
| X | CUL4B | cullin 4B | 158290 | 005893 | 232119 | 45, 38, 26 |
| X | CMC4 | C-X9-C motif containing 4 | 182712 | 165775 | 185515 | 10, 9, 22 |
| X | CXCR3 | C-X-C motif chemokine receptor 3 | 186810 | 147174 | 283599 | 36, 5, 25 |
| X | CNGA2 | cyclic nucleotide gated channel alpha 2 | 183862 | 147378 | 147381 | 38, |
| X | CCNB3 | cyclin B3 | 147082 | 147081 | 274588 | |
| X | CDK16 | cyclin dependent kinase 16 | 102225 | 130985 | 102226 | 9, 15, 40 |
| X | CDKL5 | cyclin dependent kinase like 5 | 008086 | 102098 | 102104 | 38, 25, 8 |
| X | CYLC1 | cylicin 1 | 183035 | 196767 | 072133 | 38, |
| X | CHIC1 | cysteine rich hydrophobic domain 1 | 204116 | 131264 | 187969 | 26, 13, 42 |
| X | CYSLTR1 | cysteinyl leukotriene receptor 1 | 173198 | 187325 | 179300 | 30, 22, 42 |
| X | CYBB | cytochrome b-245 beta chain | 165168 | 047597 | 165169 | 22, 5, 30 |
| X | COX7B | cytochrome c oxidase subunit 7B | 131174 | 102158 | 165240 | 29, 11, 25 |
| X | CRLF2 | cytokine receptor-like factor 2 | 205755 | 185960 | 198223 | |
| X | DACH2 | dachshund family transcription factor 2 | 126733 | 188419 | 102271 | 25, 28, 8 |
| X | DCAF12L1 | DDB1 and CUL4 associated factor 12 like 1 | 198889 | 198354 | 183631 | 45, 38, 17 |
| X | DCAF12L2 | DDB1 and CUL4 associated factor 12 like 2 | 198354 | 282815 | 198889 | 33, 15, 38 |
| X | DCAF8L1 | DDB1 and CUL4 associated factor 8 like 1 | 226372 | 177689 | 169306 | 38, |
| X | DCAF8L2 | DDB1 and CUL4 associated factor 8 like 2 | 189186 | 224960 | 177689 | |
| X | DDX3X | DEAD-box helicase 3, X-linked | 215301 | 124486 | 188937 | 42, 44, 39 |
| X | DDX53 | DEAD-box helicase 53 | 184735 | 175809 | 165186 | 38, |
| X | DOCK11 | dedicator of cytokinesis 11 | 147251 | 131725 | 131724 | 22, 1, 11 |
| X | DHRSX | dehydrogenase/reductase X-linked | 169084 | 196433 | 214717 | 34, 28, 1 |
| X | DNASE1L1 | deoxyribonuclease 1 like 1 | 013563 | 147403 | 102125 | 21, 6, 9 |
| X | DGKK | diacylglycerol kinase kappa | 274588 | 147082 | 158352 | 27, 38, 12 |
| X | DGAT2L6 | diacylglycerol O-acyltransferase 2 like 6 | 184210 | 089289 | 204195 | 35, |
| X | DIAPH2 | diaphanous related formin 2 | 147202 | 179083 | 204086 | 13, 10, 18 |
| X | DLG3 | discs large MAGUK scaffold protein 3 | 082458 | 130055 | 120498 | 28, 3, 40 |
| X | DMRTC1 | DMRT like family C1 | 269502 | 225396 | 283594 | 31, 40, 38 |
| X | DMRTC1B | DMRT like family C1B | 184911 | 067177 | 268994 | |
| X | POLA1 | DNA polymerase alpha 1, catalytic subunit | 101868 | 102230 | 004848 | 9, 7, 19 |
| X | DCX | doublecortin | 077279 | 077274 | 260802 | 8, 14, 45 |
| X | DUSP21 | dual specificity phosphatase 21 | 189037 | 069509 | 147050 | 38, |
| X | DUSP9 | dual specificity phosphatase 9 | 130829 | 262919 | 130822 | 19, 32, 17 |
| X | DYNLT3 | dynein light chain Tctex-type 3 | 165169 | 165168 | 187516 | 13, 43, 17 |
| X | DKC1 | dyskerin pseudouridine synthase 1 | 130826 | 160219 | 130830 | 21, 13, 1 |
| X | DMD | dystrophin | 198947 | 132446 | 185448 | 11, 42, 25 |
| X | DRP2 | dystrophin related protein 2 | 102385 | 102384 | 102387 | 26, 8, 25 |
| X | ELF4 | E74 like ETS transcription factor 4 | 102034 | 085185 | 156709 | 21, 5, 4 |
| X | EDA | ectodysplasin A | 158813 | 130054 | 147160 | 36, 40, 35 |
| X | EDA2R | ectodysplasin A2 receptor | 131080 | 089472 | 169083 | 40, 12, 39 |
| X | ENOX2 | ecto-NOX disulfide-thiol exchanger 2 | 165675 | 134597 | 147256 | 31, 24, 42 |
| X | EFHC2 | EF-hand domain containing 2 | 183690 | 124479 | 069509 | 42, 31, 38 |
| X | EGFL6 | EGF like domain multiple 6 | 198759 | 123594 | 176896 | 32, 11, 15 |
| X | ELK1 | ELK1, ETS transcription factor | 126767 | 126759 | 126756 | 9, 14, 40 |
| X | EMD | emerin | 102119 | 196924 | 147403 | 35, 6, 29 |
| X | EBP | emopamil binding protein (sterol isomerase) | 147155 | 102312 | 068354 | 19, 5, 33 |
| X | EFNB1 | ephrin B1 | 090776 | 130052 | 181191 | 3, 13, 16 |
| X | ERCC6L | ERCC excision repair 6 like, spindle assembly checkpoint helicase | 186871 | 102309 | 198034 | 23, 22, 6 |
| X | ERAS | ES cell expressed Ras | 187682 | 094631 | 102109 | 38, |
| X | ESX1 | ESX homeobox 1 | 123576 | 123575 | 189108 | 38, 8, |
| X | EIF1AX | eukaryotic translation initiation factor 1A, X-linked | 173674 | 184368 | 177189 | 3, 4, 2 |
| X | EIF2S3 | eukaryotic translation initiation factor 2 subunit gamma | 130741 | 174010 | 005889 | 9, 21, 42 |
| X | FAM104B | family with sequence similarity 104 member B | 182518 | 234068 | 256045 | 1, 42, 19 |
| X | FAM120C | family with sequence similarity 1200 | 184083 | 172943 | 196632 | 4, 26, 31 |
| X | FAM122B | family with sequence similarity 122B | 156504 | 170965 | 156500 | 31, 45, 1 |
| X | FAM122C | family with sequence similarity 122C | 156500 | 156504 | 101928 | 38, 29, 9 |
| X | FAM127A | family with sequence similarity 127 member A | 134590 | 212747 | 203950 | 34, 6, 35 |
| X | FAM127B | family with sequence similarity 127 member B | 203950 | 134590 | 196972 | 23, 33, 30 |
| X | FAM127C | family with sequence similarity 127 member C | 212747 | 184785 | 134590 | 1, 30, 23 |
| X | FAM133A | family with sequence similarity 133 member A | 179083 | 186310 | 147202 | 2, 38, 23 |
| X | FAM155B | family with sequence similarity 155 member B | 130054 | 181191 | 158813 | 40, 33, 14 |
| X | FAM156A | family with sequence similarity 156 member A | 268350 | 179304 | 184194 | |
| X | FAM156B | family with sequence similarity 156 member B | 179304 | 171402 | 268350 | |
| X | FAM199X | family with sequence similarity 199, X-linked | 123575 | 166707 | 123576 | 22, 29, 40 |
| X | FAM3A | family with sequence similarity 3 member A | 071889 | 126903 | 160211 | 1, 9, 28 |
| X | FAM46D | family with sequence similarity 46 member D | 174016 | 122145 | 165288 | 45, 38, 25 |
| X | FAM47A | family with sequence similarity 47 member A | 185448 | 198947 | 147027 | 38, |
| X | FAM47B | family with sequence similarity 47 member B | 189132 | 147027 | 189023 | 38, 8, |
| X | FAM47C | family with sequence similarity 47 member C | 198173 | 165164 | 130962 | 38, |
| X | FAM50A | family with sequence similarity 50 member A | 071859 | 203879 | 130827 | 7, 35, 6 |
| X | FAM58A | family with sequence similarity 58 member A | 262919 | 067842 | 130829 | 34, 41, 13 |
| X | FAM9A | family with sequence similarity 9 member A | 183304 | 011201 | 177138 | |
| X | FAM9B | family with sequence similarity 9 member B | 177138 | 183304 | 101849 | 9, 6, 38 |
| X | FAM9C | family with sequence similarity 9 member C | 187268 | 205542 | 123594 | 36, 37, 38 |
| X | FANCB | Fanconi anemia complementation group B | 181544 | 101958 | 130150 | 22, 45, 19 |
| X | FAAH2 | fatty acid amide hydrolase 2 | 165591 | 147059 | 198455 | |
| X | FRMPD3 | FERM and PDZ domain containing 3 | 147234 | 080572 | 147224 | 38, 8, |
| X | FRMPD4 | FERM and PDZ domain containing 4 | 169933 | 005302 | 101911 | 29, 26, 28 |
| X | FRMD7 | FERM domain containing 7 | 165694 | 134602 | 123728 | 17, 38, |
| X | FTHL17 | ferritin heavy chain like 17 | 132446 | 157625 | 198947 | |
| X | FATE1 | fetal and adult testis expressed 1 | 147378 | 130032 | 183862 | 38, 2, 8 |
| X | FGF13 | fibroblast growth factor 13 | 129682 | 156925 | 283147 | 34, 8, 12 |
| X | FGF16 | fibroblast growth factor 16 | 196468 | 198934 | 085224 | |
| X | FLNA | filamin A | 196924 | 007350 | 102119 | 18, 13, 1 |
| X | FOXO4 | forkhead box O4 | 184481 | 147164 | 204165 | 32, 29, 18 |
| X | FOXP3 | forkhead box P3 | 049768 | 101997 | 049769 | 36, 39, 3 |
| X | FOXR2 | forkhead box R2 | 189299 | 247746 | 083750 | 23, 10, 35 |
| X | FHL1 | four and a half UM domains 1 | 022267 | 198689 | 129680 | 24, 15, 25 |
| X | FMR1 | fragile X mental retardation 1 | 102081 | 224440 | 176988 | 22, 17, 44 |
| X | FMR1NB | fragile X mental retardation 1 neighbor | 176988 | 102081 | 155966 | 38, 8, |
| X | FTSJ1 | FtsJ RNA methyltransferase homolog 1 (E. coli) | 068438 | 017483 | 102312 | 13, 9, 37 |
| X | FUNDC1 | FUN14 domain containing 1 | 069509 | 183690 | 189037 | 3, 12, 30 |
| X | FUNDC2 | FUN14 domain containing 2 | 165775 | 277203 | 214827 | 43, 25, 26 |
| X | FGD1 | FYVE, RhoGEF and PH domain containing 1 | 102302 | 158526 | 130119 | 34, 18, 12 |
| X | GAGE1 | G antigen 1 | 205777 | 224902 | 189064 | |
| X | GAGE10 | G antigen 10 | 215274 | 049769 | 224659 | |
| X | GAGE12B | G antigen 12B | 236737 | 275113 | 237671 | 19, 10, |
| X | GAGE12C | G antigen 12C | 237671 | 236737 | 227488 | |
| X | GAGE12D | G antigen 12D | 227488 | 237671 | 216649 | 10, |
| X | GAGE12E | G antigen 12E | 216649 | 227488 | 236362 | |
| X | GAGE12F | G antigen 12F | 236362 | 216649 | 215269 | 10, 33, 38 |
| X | GAGE12G | G antigen 12G | 215269 | 236362 | 224902 | 10, 33, 38 |
| X | GAGE12H | G antigen 12H | 224902 | 215269 | 205777 | 38, |
| X | GAGE12J | G antigen 12J | 224659 | 215274 | 274274 | |
| X | GAGE13 | G antigen 13 | 274274 | 224659 | 275113 | |
| X | GAGE2A | G antigen 2A | 189064 | 205777 | 068985 | |
| X | GAGE2E | G antigen 2E | 275113 | 274274 | 236737 | 38, |
| X | GNL3L | G protein nucleolar 3 like | 130119 | 102302 | 102313 | 36, 9, 24 |
| X | GPR101 | G protein-coupled receptor 101 | 165370 | 147274 | 156925 | 33, |
| X | GPR119 | G protein-coupled receptor 119 | 147262 | 102078 | 134597 | 28, |
| X | GPR143 | G protein-coupled receptor 143 | 101850 | 101849 | 146950 | |
| X | GPR173 | G protein-coupled receptor 173 | 184194 | 268350 | 184205 | 6, 14, 28 |
| X | GPR174 | G protein-coupled receptor 174 | 147138 | 078589 | 078596 | 22, 39, |
| X | GPR34 | G protein-coupled receptor 34 | 171659 | 147044 | 171657 | 1, 32, 42 |
| X | GPR50 | G protein-coupled receptor 50 | 102195 | 029993 | 160131 | 17, 12, |
| X | GPR82 | G protein-coupled receptor 82 | 171657 | 171659 | 279849 | 22, 16, 32 |
| X | GPRASP1 | G protein-coupled receptor associated sorting protein 1 | 198932 | 125962 | 158301 | 1, 26, 25 |
| X | GPRASP2 | G protein-coupled receptor associated sorting protein 2 | 158301 | 198932 | 198908 | 8, 42, 38 |
| X | GSPT2 | G1 to S phase transition 2 | 189369 | 223591 | 179222 | 15, 38, 36 |
| X | GLA | galactosidase alpha | 102393 | 257529 | 126945 | 20, 3, 11 |
| X | GABRA3 | gamma-aminobutyric acid type A receptor alpha3 subunit | 011677 | 124260 | 268089 | 18, 8, 10 |
| X | GABRE | gamma-aminobutyric acid type A receptor epsilon subunit | 102287 | 147381 | 124260 | 32, 44, 35 |
| X | GABRQ | gamma-aminobutyric acid type A receptor theta subunit | 268089 | 011677 | 221867 | 27, 8, |
| X | GJB1 | gap junction protein beta 1 | 169562 | 196338 | 147130 | 35, 37, 16 |
| X | GRPR | gastrin releasing peptide receptor | 126010 | 182287 | 182798 | 12, 23, 16 |
| X | GATA1 | GATA binding protein 1 | 102145 | 171433 | 094631 | 7, 34, 25 |
| X | GDI1 | GDP dissociation inhibitor 1 | 203879 | 071553 | 071859 | 8, 36, 39 |
| X | GEMIN8 | gem nuclear organelle associated protein 8 | 046647 | 046653 | 101958 | 34, 29, 15 |
| X | G6PD | glucose-6-phosphate dehydrogenase | 160211 | 071889 | 269335 | 7, 13, 41 |
| X | GLUD2 | glutamate dehydrogenase 2 | 182890 | 236371 | 125675 | 5, 35, 21 |
| X | GRIA3 | glutamate ionotropic receptor AMPA type subunit 3 | 125675 | 182890 | 125676 | 8, 2, 25 |
| X | GK | glycerol kinase | 198814 | 120280 | 157625 | 42, 29, 30 |
| X | GDPD2 | glycerophosphodiester phosphodiesterase domain containing 2 | 130055 | 090889 | 082458 | 36, 12, 8 |
| X | GLRA2 | glycine receptor alpha 2 | 101958 | 046647 | 181544 | 8, 27, 38 |
| X | GLRA4 | glycine receptor alpha 4 | 188828 | 123562 | 179363 | 12, |
| X | GYG2 | glycogenin 2 | 056998 | 124343 | 006756 | 1, 3, 35 |
| X | GPM6B | glycoprotein M6B | 046653 | 046651 | 046647 | 11, 8, 26 |
| X | GLOD5 | glyoxalase domain containing 5 | 171433 | 101945 | 102145 | 16, 17, 20 |
| X | GPC3 | glypican 3 | 147257 | 076716 | 203952 | 43, 32, 19 |
| X | GPC4 | glypican 4 | 076716 | 183434 | 147257 | 12, 31, 15 |
| X | GPKOW | G-patch domain and KOW motifs | 068394 | 196998 | 280116 | 9, 3, 5 |
| X | GAB3 | GRB2 associated binding protein 3 | 160219 | 126890 | 130826 | 45, 22, 42 |
| X | GRIPAP1 | GRIP1 associated protein 1 | 068400 | 102057 | 068323 | 6, 35, 18 |
| X | GTPBP6 | GTP binding protein 6 (putative) | 178605 | 182378 | 167393 | 1, 34, 20 |
| X | GUCY2F | guanylate cyclase 2F, retinal | 101890 | 133124 | 101888 | 14, |
| X | H2AFB1 | H2A histone family member B1 | 274183 | 185010 | 277203 | 38, |
| X | H2AFB2 | H2A histone family member B2 | 277858 | 155962 | 274791 | 38, 14, |
| X | H2AFB3 | H2A histone family member B3 | 277745 | 277150 | 185973 | |
| X | H2BFM | H2B histone family member M | 101812 | 123569 | 269226 | 38, |
| X | H2BFWT | H2B histone family member W, testis specific | 123569 | 158427 | 101812 | 38, |
| X | HAUS7 | HAUS augmin like complex subunit 7 | 213397 | 183479 | 182492 | 3, 9, 21 |
| X | HSFX1 | heat shock transcription factor family, X-linked 1 | 171116 | 185247 | 123584 | |
| X | HSFX2 | heat shock transcription factor family, X-linked 2 | 268738 | 267978 | 269556 | 38, 14, 27 |
| X | HUWE1 | HECT, UBA and WWE domain containing 1, E3 ubiquitin protein ligase | 086758 | 072506 | 172943 | 18, 43, 31 |
| X | HS6ST2 | heparan sulfate 6-O-sulfotransferase 2 | 171004 | 076770 | 134588 | 24, 4, 38 |
| X | HEPH | hephaestin | 089472 | 155659 | 131080 | 11, 1, 16 |
| X | HNRNPH2 | heterogeneous nuclear ribonucleoprotein H2 | 126945 | 102393 | 196440 | |
| X | HMGB3 | high mobility group box 3 | 029993 | 102181 | 102195 | 29, 12, 37 |
| X | HMGN5 | high mobility group nucleosome binding domain 5 | 198157 | 165288 | 131171 | 7, 9, 11 |
| X | HDX | highly divergent homeobox | 165259 | 072133 | 155008 | 11, 24, 45 |
| X | HDAC6 | histone deacetylase 6 | 094631 | 102145 | 187682 | 43, 39, 38 |
| X | HDAC8 | histone deacetylase 8 | 147099 | 125931 | 067177 | 21, 9, 5 |
| X | HTATSF1 | HIV-1 Tat specific factor 1 | 102241 | 102239 | 102243 | 31, 18, 17 |
| X | HCCS | holocytochrome c synthase | 004961 | 101871 | 047648 | 41, 45, 29 |
| X | HCFC1 | host cell factor C1 | 172534 | 102032 | 177854 | 21, 1, 27 |
| X | HYPM | huntingtin interacting protein M | 187516 | 165169 | 147041 | 38, |
| X | HSD17B10 | hydroxysteroid 17-beta dehydrogenase 10 | 072506 | 158423 | 086758 | 29, 9, 27 |
| X | HPRT1 | hypoxanthine phosphoribosyltransferase 1 | 165704 | 156531 | 170965 | 31, 22, 9 |
| X | IDS | iduronate 2-sulfatase | 010404 | 155966 | 241489 | 8, 31, 28 |
| X | IGBP1 | immunoglobulin (CD79A) binding protein 1 | 089289 | 189401 | 184210 | 9, 5, 21 |
| X | IGSF1 | immunoglobulin superfamily member 1 | 147255 | 147256 | 171054 | 31, 40, 38 |
| X | IKBKG | inhibitor of kappa light polypeptide gene enhancer in B-cells, kinase gamma | 269335 | 160211 | 268651 | |
| X | IRS4 | insulin receptor substrate 4 | 133124 | 260548 | 101890 | 2, 17, 14 |
| X | ITM2A | integral membrane protein 2A | 078596 | 147138 | 122145 | |
| X | INTS6L | integrator complex subunit 6 like | 165359 | 178947 | 268940 | |
| X | ITGB1BP2 | integrin subunit beta 1 binding protein 2 | 147166 | 147140 | 147133 | 43, 15, 25 |
| X | ITIH6 | inter-alpha-trypsin inhibitor heavy chain family member 6 | 102313 | 130119 | 102316 | 42, 35, 6 |
| X | IL1RAPL1 | interleukin 1 receptor accessory protein like 1 | 169306 | 226372 | 099399 | 11, 39, 33 |
| X | IL1RAPL2 | interleukin 1 receptor accessory protein like 2 | 189108 | 123576 | 268629 | 19, 38, 8 |
| X | IRAK1 | interleukin 1 receptor associated kinase 1 | 184216 | 177854 | 169057 | 9, 35, 1 |
| X | IL13RA1 | interleukin 13 receptor subunit alpha 1 | 131724 | 147251 | 174460 | 40, 4, 27 |
| X | IL13RA2 | interleukin 13 receptor subunit alpha 2 | 123496 | 147246 | 130224 | 1, 38, 2 |
| X | IL2RG | interleukin 2 receptor subunit gamma | 147168 | 204165 | 184634 | 5, 39, 41 |
| X | IL3RA | interleukin 3 receptor subunit alpha | 185291 | 198223 | 169100 | 1, 18, 2 |
| X | IL9R | interleukin 9 receptor | 124334 | 124333 | 102054 | |
| X | IQSEC2 | IQ motif and Sec7 domain 2 | 124313 | 126012 | 072501 | 34, 6, 3 |
| X | IDH3G | isocitrate dehydrogenase 3 (NAD(+)) gamma | 067829 | 184343 | 180879 | 23, 26, 41 |
| X | JADE3 | jade family PHD finger 3 | 102221 | 102218 | 130988 | 4, 39, 28 |
| X | KLHL13 | kelch like family member 13 | 003096 | 204019 | 131725 | 1, 42, 38 |
| X | KLHL15 | kelch like family member 15 | 174010 | 165182 | 130741 | 18, 29, 26 |
| X | KLHL34 | kelch like family member 34 | 185915 | 149970 | 091482 | 24, 36, 25 |
| X | KLHL4 | kelch like family member 4 | 102271 | 126733 | 147183 | 38, 8, 45 |
| X | KIAA1210 | KIAA1210 | 250423 | 277535 | 101856 | 27, 38, 33 |
| X | KIAA2022 | KIAA2022 | 050030 | 131263 | 131269 | 42, 25, 8 |
| X | KIF4A | kinesin family member 4A | 090889 | 120509 | 130055 | 9, 22, 21 |
| X | KRBOX4 | KRAB box domain containing 4 | 147121 | 147113 | 251192 | 4, 39, 15 |
| X | KLF8 | Kruppel like factor 8 | 102349 | 083750 | 188021 | 18, 30, 26 |
| X | LAGE3 | L antigen family member 3 | 196976 | 130827 | 102178 | 20, 2, 33 |
| X | L1CAM | L1 cell adhesion molecule | 198910 | 067840 | 126895 | 26, 9, 8 |
| X | LANCL3 | LanC like 3 | 147036 | 250349 | 047597 | 27, 5, 19 |
| X | LAS1L | LAS1 like, ribosome biogenesis factor | 001497 | 102053 | 147065 | 3, 13, 20 |
| X | LRCH2 | leucine rich repeats and calponin homology domain containing 2 | 130224 | 123496 | 175718 | 18, 31, 45 |
| X | LDOC1 | leucine zipper down-regulated in cancer 1 | 182195 | 227234 | 198573 | 41, 31, 1 |
| X | LUZP4 | leucine zipper protein 4 | 102021 | 175718 | 102024 | 38, |
| X | LHFPL1 | lipoma HMGIC fusion partner-like 1 | 182508 | 187823 | 126016 | 22, 30, 32 |
| X | LONRF3 | LON peptidase N-terminal domain and ring finger 3 | 175556 | 174460 | 277535 | 40, 4, 16 |
| X | LINC01420 | Long intergenic non-protein coding RNA 1420 | 204272 | 188021 | 204271 | 30, 31, 10 |
| X | LINC00633 | Long intergenic non-protein coding RNA 633 | 224107 | 196972 | 169551 | 38, |
| X | LINC00684 | Long intergenic non-protein coding RNA 684 | 275520 | 283594 | 184388 | 38, |
| X | LINC00890 | Long intergenic non-protein coding RNA 890 | 260802 | 077279 | 101901 | 25, 44, 38 |
| X | KDM5C | lysine demethylase 5C | 126012 | 184205 | 124313 | 37, 22, 24 |
| X | KDM6A | lysine demethylase 6A | 147050 | 189037 | 147113 | 1, 15, 13 |
| X | LPAR4 | lysophosphatidic acid receptor 4 | 147145 | 179300 | 078589 | 38, 12, 39 |
| X | LAMP2 | lysosomal associated membrane protein 2 | 005893 | 101892 | 158290 | 42, 8, 24 |
| X | MAGEA1 | MAGE family member A1 | 198681 | 235961 | 225110 | 6, 23, 35 |
| X | MAGEA10 | MAGE family member A10 | 124260 | 102287 | 011677 | 35, 32, 24 |
| X | MAGEA11 | MAGE family member A11 | 185247 | 269556 | 171116 | 13, 32, 6 |
| X | MAGEA12 | MAGE family member A12 | 213401 | 198930 | 268606 | 35, 2, 33 |
| X | MAGEA2 | MAGE family member A2 | 268606 | 213401 | 197172 | |
| X | MAGEA2B | MAGE family member A2B | 183305 | 221867 | 198930 | 2, 23, 33 |
| X | MAGEA3 | MAGE family member A3 | 221867 | 268089 | 183305 | 35, 34, 33 |
| X | MAGEA4 | MAGE family member A4 | 147381 | 183862 | 102287 | 16, 37, 19 |
| X | MAGEA6 | MAGE family member A6 | 197172 | 268606 | 147400 | 35, 2, 32 |
| X | MAGEA8 | MAGE family member A8 | 156009 | 123584 | 197021 | 32, 35, 44 |
| X | MAGEA9 | MAGE family member A9 | 123584 | 171116 | 156009 | |
| X | MAGEA9B | MAGE family member A9B | 267978 | 283697 | 268738 | 13, 4, 6 |
| X | MAGEB1 | MAGE family member B1 | 214107 | 120289 | 169297 | 34, 35, 38 |
| X | MAGEB10 | MAGE family member B10 | 177689 | 189186 | 226372 | 38, |
| X | MAGEB16 | MAGE family member B16 | 189023 | 189132 | 165164 | |
| X | MAGEB17 | MAGE family member B17 | 182798 | 126010 | 047230 | |
| X | MAGEB18 | MAGE family member B18 | 176774 | 004848 | 232030 | 38, |
| X | MAGEB2 | MAGE family member B2 | 099399 | 169306 | 198798 | 6, 5, 35 |
| X | MAGEB3 | MAGE family member B3 | 198798 | 099399 | 120289 | 38, 33, |
| X | MAGEB4 | MAGE family member B4 | 120289 | 198798 | 214107 | 38, |
| X | MAGEB5 | MAGE family member B5 | 188408 | 176746 | 224960 | |
| X | MAGEB6 | MAGE family member B6 | 176746 | 232030 | 188408 | 38, |
| X | MAGEB6P1 | MAGE family member B6 pseudogene 1 | 232030 | 176774 | 176746 | |
| X | MAGEC1 | MAGE family member C1 | 155495 | 165509 | 046774 | 35, 6, 3 |
| X | MAGEC2 | MAGE family member C2 | 046774 | 155495 | 189326 | 35, 34, 38 |
| X | MAGEC3 | MAGE family member C3 | 165509 | 196406 | 155495 | 8, |
| X | MAGED1 | MAGE family member D1 | 179222 | 189369 | 187243 | 35, 34, 31 |
| X | MAGED2 | MAGE family member D2 | 102316 | 102313 | 067445 | 31, 26, 35 |
| X | MAGED4 | MAGE family member D4 | 154545 | 187243 | 155622 | |
| X | MAGED4B | MAGE family member D4B | 187243 | 179222 | 154545 | |
| X | MAGEE1 | MAGE family member E1 | 198934 | 102390 | 196468 | 8, 26, 11 |
| X | MAGEE2 | MAGE family member E2 | 186675 | 102383 | 102390 | 8, |
| X | MAGEH1 | MAGE family member H1 | 187601 | 204279 | 247746 | 31, 1, 26 |
| X | MAGIX | MAGI family member, X-linked | 269313 | 280116 | 102007 | 29, 31, 2 |
| X | MAGT1 | magnesium transporter 1 | 102158 | 085224 | 131174 | 42, 45, 24 |
| X | MSL3 | male-specific lethal 3 homolog (Drosophila) | 005302 | 125363 | 169933 | 39, 36, 5 |
| X | MAP7D2 | MAP7 domain containing 2 | 184368 | 173681 | 173674 | 8, 19, 22 |
| X | MAP7D3 | MAP7 domain containing 3 | 129680 | 022267 | 156920 | 31, 40, 17 |
| X | MAMLD1 | mastermind like domain containing 1 | 013619 | 283463 | 171100 | 11, 40, 1 |
| X | MXRA5 | matrix remodeling associated 5 | 101825 | 062096 | 183943 | 11, 1, 40 |
| X | MTCP1 | mature T-cell proliferation 1 | 214827 | 165775 | 185515 | 10, 9, 22 |
| X | MCF2 | MCF.2 cell line derived transforming sequence | 101977 | 101981 | 101974 | 31, 38, 2 |
| X | MCTS1 | MCTS1, re-initiation and release factor | 232119 | 158290 | 171155 | 4, 7, 43 |
| X | MED12 | mediator complex subunit 12 | 184634 | 147168 | 196338 | 7, 43, 22 |
| X | MED14 | mediator complex subunit 14 | 180182 | 185753 | 124486 | 22, 24, 40 |
| X | MBTPS2 | membrane bound transcription factor peptidase, site 2 | 012174 | 091482 | 230797 | 42, 26, 38 |
| X | MMGT1 | membrane magnesium transporter 1 | 169446 | 181433 | 198689 | 42, 39, 29 |
| X | MPP1 | membrane palmitoylated protein 1 | 130830 | 130826 | 203870 | 39, 36, 5 |
| X | MECP2 | methyl-CpG binding protein 2 | 169057 | 184216 | 102076 | 6, 1, 26 |
| X | MID1IP1 | MIDI interacting protein 1 | 165175 | 156298 | 183337 | 3, 13, 5 |
| X | MIDI | midline 1 | 101871 | 073464 | 004961 | 42, 24, 29 |
| X | MID2 | midline 2 | 080561 | 170935 | 170925 | 10, 11, 42 |
| X | MPC1L | mitochondrial pyruvate carrier 1-like | 238205 | 182220 | 185753 | 38, |
| X | MAP3K15 | mitogen-activated protein kinase kinase kinase 15 | 180815 | 131828 | 147010 | 40, 2, 17 |
| X | MSN | moesin | 147065 | 001497 | 155659 | |
| X | MAOA | monoamine oxidase A | 189221 | 102055 | 069535 | 26, 1, 42 |
| X | MAOB | monoamine oxidase B | 069535 | 189221 | 124479 | 26, 2, 17 |
| X | MORC4 | MORO family CW-type zinc finger 4 | 133131 | 133138 | 147223 | 13, 32, 33 |
| X | MORF4L2 | mortality factor 4 like 2 | 123562 | 172465 | 188828 | 13, 31, 9 |
| X | MOSPD1 | motile sperm domain containing 1 | 101928 | 156500 | 184785 | 4, 13, 45 |
| X | MOSPD2 | motile sperm domain containing 2 | 130150 | 181544 | 102048 | 22, 4, 1 |
| X | MTRNR2L10 | MT-RNR2-like 10 | 256045 | 182518 | 158639 | 8, |
| X | MUM1L1 | MUM1 like 1 | 157502 | 123561 | 147231 | 38, 44, 27 |
| X | MBNL3 | muscleblind like splicing regulator 3 | 076770 | 123728 | 171004 | 32, 31, 22 |
| X | MTM1 | myotubularin 1 | 171100 | 013619 | 063601 | 42, 19, 22 |
| X | MTMR1 | myotubularin related protein 1 | 063601 | 171100 | 102181 | 22, 26, 40 |
| X | MTMR8 | myotubularin related protein 8 | 102043 | 198881 | 126970 | 27, 36, 44 |
| X | NAA10 | N(alpha)-acetyltransferase 10, NatA catalytic subunit | 102030 | 089820 | 102032 | 31, 9, 2 |
| X | NSDHL | NAD(P) dependent steroid dehydrogenase-like | 147383 | 147400 | 147394 | 2, 3, 34 |
| X | NDUFA1 | NADH:ubiquinone oxidoreductase subunit A1 | 125356 | 125352 | 186471 | 25, 15, 19 |
| X | NDUFB11 | NADH:ubiquinone oxidoreductase subunit B11 | 147123 | 130988 | 182872 | 29, 15, 25 |
| X | NOX1 | NADPH oxidase 1 | 007952 | 101811 | 182489 | 16, 23, |
| X | NDP | NDP, nonin cystine knot growth factor | 124479 | 069535 | 183690 | 44, 14, 8 |
| X | NLGN3 | neuroligin 3 | 196338 | 184634 | 169562 | 8, 38, 2 |
| X | NLGN4X | neuroligin 4, X-linked | 146938 | 183943 | 169059 | 26, 4, 31 |
| X | NKAP | NFKB activating protein | 101882 | 186471 | 203989 | 11, 41, 9 |
| X | NKRF | NFKB repressing factor | 186416 | 077721 | 125354 | 13, 9, 11 |
| X | NHS | NHS actin remodeling regulator | 188158 | 169891 | 047634 | 11, 37, 14 |
| X | NHSL2 | NHS like 2 | 204131 | 215113 | 242732 | |
| X | NRK | Nik related kinase | 123572 | 268629 | 123561 | 32, 15, 12 |
| X | NLRP2B | NLR family pyrin domain containing 2B | 215174 | 198455 | 198205 | |
| X | NONO | non-POU domain containing, octamer-binding | 147140 | 147130 | 147166 | |
| X | NCBP2L | nuclear cap binding protein subunit 2-like | 170935 | 157514 | 080561 | |
| X | NR0B1 | nuclear receptor subfamily 0 group B member 1 | 169297 | 214107 | 120280 | 2, 20, 4 |
| X | NXF2 | nuclear RNA export factor 2 | 269405 | 184515 | 269437 | |
| X | NXF2B | nuclear RNA export factor 2B | 269437 | 269405 | 215029 | 38, 18, 35 |
| X | NXF3 | nuclear RNA export factor 3 | 147206 | 133169 | 102409 | 38, 3, 27 |
| X | NXF5 | nuclear RNA export factor 5 | 126952 | 184867 | 166432 | |
| X | NXT2 | nuclear transport factor 2 like export factor 2 | 101888 | 101890 | 176076 | 24, 38, 32 |
| X | NUP62CL | nudeoporin 62 C-terminal like | 198088 | 089682 | 080572 | 34, 5, 39 |
| X | NAP1L2 | nucleosome assembly protein 1 like 2 | 186462 | 204118 | 131264 | 26, 43, 8 |
| X | NAP1L3 | nucleosome assembly protein 1 like 3 | 186310 | 102290 | 179083 | 8, 26, 17 |
| X | NAP1L6 | Nucleosome assembly protein 1 like 6 | 204118 | 186288 | 186462 | 14, 8, |
| X | NUDT10 | nudix hydrolase 10 | 122824 | 130385 | 187690 | 18, 12, 2 |
| X | NUDT11 | nudix hydrolase 11 | 196368 | 187690 | 224109 | 45, 38, 28 |
| X | NYX | nyctalopin | 188937 | 215301 | 147044 | 17, |
| X | OCRL | OCRL, inositol polyphosphate-5-phosphatase | 122126 | 102038 | 171388 | 44, 3, 9 |
| X | OFD1 | OFD1, centriole and centriolar satellite protein | 046651 | 196459 | 046653 | |
| X | OR13H1 | olfactory receptor family 13 subfamily H member 1 | 171054 | 147255 | 134602 | |
| X | OPHN1 | oligophrenin 1 | 079482 | 169083 | 181704 | 1, 23, 32 |
| X | OGT | O-linked N-acetylglucosamine (GlcNAc) transferase | 147162 | 147133 | 147174 | 24, 39, 18 |
| X | OPN1LW | opsin 1 (cone pigments), long-wave-sensitive | 102076 | 169057 | 268221 | 14, |
| X | OPN1MW | opsin 1 (cone pigments), medium-wave-sensitive | 268221 | 102076 | 166160 | 36, 14, |
| X | OPN1MW2 | opsin 1 (cone pigments), medium-wave-sensitive 2 | 166160 | 268221 | 269433 | 14, |
| X | OPN1MW3 | opsin 1 (cone pigments), medium-wave-sensitive 3 | 269433 | 166160 | 278057 | 36, 14, |
| X | OTC | ornithine carbamoyltransferase | 036473 | 156313 | 156298 | 19, 10, 38 |
| X | OTUD5 | OTU deubiquitinase 5 | 068308 | 102096 | 102057 | 34, 41, 9 |
| X | OTUD6A | OTU deubiquitinase 6A | 189401 | 147160 | 089289 | 38, |
| X | PAK3 | p21 (RAC1) activated kinase 3 | 077264 | 101938 | 077274 | 31, 28, 36 |
| X | PAGE1 | PAGE family member 1 | 068985 | 189064 | 101951 | 35, 17, 6 |
| X | PAGE2 | PAGE family member 2 | 234068 | 238269 | 182518 | 38, 37, 10 |
| X | PAGE2B | PAGE family member 2B | 238269 | 158578 | 234068 | 3, 37, 38 |
| X | PAGE3 | PAGE family member 3 | 204279 | 158639 | 187601 | 38, 8, |
| X | PAGE4 | PAGE family member 4 | 101951 | 068985 | 273820 | 32, 33, 15 |
| X | PAGE5 | PAGE family member 5 | 158639 | 256045 | 204279 | 35, 38, 32 |
| X | PNMA3 | paranecplastic Ma antigen 3 | 183837 | 198883 | 235961 | |
| X | PNMA5 | paranecplastic Ma antigen family member 5 | 198883 | 147394 | 183837 | 18, 38, 8 |
| X | PNMA6A | paranecplastic Ma antigen family member 6A | 235961 | 183837 | 198681 | 11, 8, 14 |
| X | PASD1 | PAS domain ccntaining 1 | 166049 | 160131 | 130032 | 38, 6, |
| X | PNPLA4 | patatin like phospholipase domain containing 4 | 006757 | 182583 | 177504 | 29, 9, 20 |
| X | PTCHD1 | patched dcmain containing 1 | 165186 | 184735 | 123131 | 27, 44, 23 |
| X | PDZD11 | PDZ domain containing 11 | 120509 | 147127 | 090889 | 4, 29, 31 |
| X | PDZD4 | PDZ domain containing 4 | 067840 | 180879 | 198910 | 26, 1, 8 |
| X | PIN4 | peptidylprolyl cis/trans isomerase, NIMA-interacting 4 | 102309 | 242732 | 186871 | 4, 17, 2 |
| X | PRDX4 | peroxiredoxin 4 | 123131 | 165186 | 123130 | 17, 45, 31 |
| X | PHF6 | PHD finger protein 6 | 156531 | 203952 | 165704 | 22, 4, 7 |
| X | PHF8 | PHD finger protein 8 | 172943 | 086758 | 184083 | 43, 38, 22 |
| X | PCYT1B | phosphate cytidylyltransferase 1, choline, beta | 102230 | 067992 | 101868 | 38, 8, 12 |
| X | PHEX | phosphate regulating endopeptidase hemeleg, X-linked | 102174 | 102172 | 175809 | 10, 42, 39 |
| X | PIGA | phosphatidylinositol glycan anchor biosynthesis class A | 165195 | 165192 | 165197 | |
| X | PLCXD1 | phosphatidylinositol specific phcsphclipase C X dcmain ccntaining 1 | 182378 | 102054 | 178605 | 22, 30, 21 |
| X | PGK1 | phosphoglycerate kinase 1 | 102144 | 226784 | 187325 | 13, 9, 3 |
| X | PGAM4 | phosphoglycerate mutase family member 4 | 226784 | 165240 | 102144 | 16, 33, 28 |
| X | PRPS1 | phosphoribosyl pyrophosphate synthetase 1 | 147224 | 147234 | 157514 | 39, 21, 36 |
| X | PRPS2 | phosphoribosyl pyrophpsphate synthetase 2 | 101911 | 169933 | 196664 | 3, 29, 38 |
| X | PHKA1 | phosphorylase kinase regulatory subunit alpha 1 | 067177 | 147099 | 184911 | 29, 11, 30 |
| X | PHKA2 | phosphorylase kinase regulatory subunit alpha 2 | 044446 | 086717 | 173698 | 22, 42, 26 |
| X | PIH1D3 | PIH1 dpmain cpntaining 3 | 080572 | 198088 | 147234 | 44, 38, 20 |
| X | PIM2 | Pim-2 protp-pncpgene, serine/threenine kinase | 102096 | 102100 | 068308 | 5, 36, 39 |
| X | PIR | pirin | 087842 | 165197 | 102010 | 10, 34, 35 |
| X | PLAC1 | placenta specific 1 | 170965 | 165704 | 156504 | 32, 10, 23 |
| X | PLS3 | plastin 3 | 102024 | 102021 | 180772 | 45, 13, 39 |
| X | PLXNA3 | plexin A3 | 130827 | 071859 | 196976 | 26, 18, 16 |
| X | PLXNB3 | plexin B3 | 198753 | 101986 | 184343 | 26, 8, 2 |
| X | PABPC1L2A | poly(A) binding protein cytoplasmic 1 like 2A | 186288 | 184388 | 204118 | 27, 33, |
| X | PABPC1L2B | poly(A) binding protein cytoplasmic 1 like 2B | 184388 | 275520 | 186288 | 27, 17, 8 |
| X | PABPC5 | poly(A) binding protein cytoplasmic 5 | 174740 | 153779 | 102290 | 10, 32, 14 |
| X | PQBP1 | polyglutamine binding protein 1 | 102103 | 126768 | 102100 | 29, 21, 3 |
| X | PBDC1 | polysaccharide biosynthesis domain containing 1 | 102390 | 186675 | 198934 | 2, 41, 37 |
| X | PORCN | porcupine homolog (Drosophila) | 102312 | 068438 | 147155 | 34, 2, 40 |
| X | KCND1 | potassium voltage-gated channel subfamily D member 1 | 102057 | 068308 | 068400 | 15, 10, 16 |
| X | KCNE5 | potassium voltage-gated channel subfamily E regulatory subunit 5 | 176076 | 101888 | 068366 | 15, 25, 39 |
| X | POU3F4 | POU class 3 homeobox 4 | 196767 | 131171 | 183035 | 8, |
| X | PRAF2 | PRA1 domain family member 2 | 243279 | 147144 | 196998 | 34, 21, 43 |
| X | PJA1 | praja ring finger ubiquitin ligase 1 | 181191 | 090776 | 130054 | 43, 8, 26 |
| X | PNCK | pregnancy up-regulated nonubiquitous CaM kinase | 130822 | 130829 | 130821 | |
| X | POF1B | premature ovarian failure, 1B | 124429 | 147180 | 188419 | 42, 4, 16 |
| X | PRICKLE3 | prickle planar cell polarity protein 3 | 012211 | 102007 | 102003 | 13, 21, 39 |
| X | PGRMC1 | progesterone receptor membrane component 1 | 101856 | 250423 | 077713 | 11, 26, 29 |
| X | PRR32 | proline rich 32 | 183631 | 198889 | 123165 | 10, 15, 20 |
| X | PRRG1 | proline rich and Gla domain 1 | 130962 | 198173 | 250349 | 42, 8, 32 |
| X | PRRG3 | proline rich and Gla domain 3 | 130032 | 166049 | 147378 | 14, 33, 27 |
| X | PCSK1N | proprotein convertase subtilisin/kexin type 1 inhibitor | 102109 | 187682 | 126768 | 28, 31, 17 |
| X | PSMD10 | proteasome 26S subunit, non-ATPase 10 | 101843 | 101842 | 101844 | 30, 34, 9 |
| X | PRKX | protein kinase, X-linked | 183943 | 101825 | 146938 | 21, 12, 3 |
| X | PPP1R2P9 | Protein phosphatase 1 regulatory inhibitor subunit 2 pseudogene 9 | 102055 | 279849 | 189221 | 38, |
| X | PPP1R3F | protein phosphatase 1 regulatory subunit 3F | 049769 | 049768 | 215274 | 14, 31, 33 |
| X | PPP2R3B | protein phosphatase 2 regulatory subunit B″beta | 167393 | 178605 | 185960 | 9, 43, 26 |
| X | PPP4R3CP | Protein phosphatase 4 regulatory subunit 30, pseudogene | 224960 | 188408 | 189186 | 38, |
| X | PPEF1 | protein phosphatase with EF-hand domain 1 | 086717 | 102104 | 044446 | 38, 2, 8 |
| X | PLP1 | proteolipid protein 1 | 123560 | 179363 | 123570 | 8, 17, 26 |
| X | PLP2 | proteolipid protein 2 | 102007 | 269313 | 012211 | 37, 9, 6 |
| X | PCDH11X | protocadherin 11 X-linked | 102290 | 174740 | 186310 | 11, 31, 14 |
| X | PCDH19 | protocadherin 19 | 165194 | 204086 | 5 | |
| X | PUDP | pseudouridine 5′-phosphatase | 130021 | 169059 | 101846 | 32, 45, 2 |
| X | P2RY10 | purinergic receptor P2Y10 | 078589 | 147145 | 147138 | 39, 5, 42 |
| X | P2RY8 | purinergic receptor P2Y8 | 182162 | 169093 | 197976 | 22, 21, 39 |
| X | P2RY4 | pyrimidinergic receptor P2Y4 | 186912 | 204195 | 120500 | |
| X | PDHA1 | pyruvate dehydrogenase (lipoamide) alpha 1 | 131828 | 173698 | 180815 | 1, 25, 40 |
| X | PDK3 | pyruvate dehydrogenase kinase 3 | 067992 | 005889 | 102230 | 31, 40, 29 |
| X | RAB33A | RAB33A, member RAS oncogene family | 134594 | 156709 | 056277 | 21, 41, 14 |
| X | RAB39B | RAB39B, member RAS oncogene family | 155961 | 155959 | 155962 | 22, 29, 28 |
| X | RAB40A | RAB40A, member RAS oncogene family | 172476 | 166681 | 133142 | 23, 20, 33 |
| X | RAB40AL | RAB40A, member RAS oncogene family-like | 102128 | 198908 | 133169 | 20, |
| X | RAB41 | RAB41, member RAS oncogene family | 147127 | 120500 | 120509 | 14, 38, 20 |
| X | RAB9A | RAB9A, member RAS oncogene family | 123595 | 176896 | 196459 | 18, 29, 1 |
| X | RAB9B | RAB9B, member RAS oncogene family | 123570 | 123560 | 158427 | 13, 8, 28 |
| X | ARHGEF6 | Rac/Cdc42 guanine nucleotide exchange factor 6 | 129675 | 102245 | 147274 | 39, 1, 42 |
| X | REPS2 | RALBP1 associated Eps domain containing 2 | 169891 | 102054 | 188158 | 29, 26, 14 |
| X | RAP2C | RAP2C, member of RAS oncogene family | 123728 | 165694 | 076770 | 22, 44, 4 |
| X | RRAGB | Ras related GTP binding B | 083750 | 189299 | 102349 | 2, 30, 25 |
| X | RBBP7 | RB binding protein 7, chromatin remodeling factor | 102054 | 086712 | 169891 | 2, 11, 21 |
| X | RGN | regucalcin | 130988 | 102221 | 147123 | 2, 31, 17 |
| X | RENBP | renin binding protein | 102032 | 102030 | 172534 | 26, 36, 35 |
| X | RPA4 | replication protein A4 | 204086 | 147202 | 165194 | 45, 38, |
| X | RP2 | retinitis pigmentosa 2 (X-linked recessive) | 102218 | 065923 | 102221 | 7, 22, 5 |
| X | RPGR | retinitis pigmentosa GTPase regulator | 156313 | 101955 | 036473 | 45, 4, 31 |
| X | RAI2 | retinoic acid induced 2 | 131831 | 047634 | 177324 | 44, 9, 20 |
| X | RS1 | retinoschisin 1 | 102104 | 008086 | 086717 | 14, 17, 10 |
| X | RGAG1 | retrotransposon gag domain containing 1 | 243978 | 101935 | 101938 | 38, 20, 35 |
| X | RGAG4 | retrotransposon gag domain containing 4 | 242732 | 204131 | 102309 | 31, 29, 40 |
| X | ARHGAP36 | Rho GTPase activating protein 36 | 147256 | 165675 | 147255 | 31, 26, 40 |
| X | ARHGAP4 | Rho GTPase activating protein 4 | 089820 | 126895 | 102030 | 7, 39, 36 |
| X | ARHGAP6 | Rho GTPase activating protein 6 | 047648 | 004961 | 125363 | 10, 33, 26 |
| X | RHOXF1 | Rhox homeobox family member 1 | 101883 | 203989 | 131721 | |
| X | RHOXF2 | Rhox homeobox family member 2 | 131721 | 101883 | 177485 | 38, 19, 44 |
| X | RHOXF2B | Rhox homeobox family member 2B | 203989 | 101882 | 101883 | 10, 38, 6 |
| X | RIBC1 | RIB43A domain with coiled-coils 1 | 158423 | 072501 | 072506 | 42, 30, 24 |
| X | RPL10 | ribosomal protein L10 | 147403 | 102119 | 013563 | 41, 21, 3 |
| X | RPL36A | ribosomal protein L36a | 241343 | 010671 | 257529 | |
| X | RPL39 | ribosomal protein L39 | 198918 | 187808 | 125351 | |
| X | RPS4X | ribosomal protein S4, X-linked | 198034 | 186871 | 125931 | 25, |
| X | RPS6KA3 | ribosomal protein S6 kinase A3 | 177189 | 173674 | 149970 | 40, 3, 45 |
| X | RPS6KA6 | ribosomal protein S6 kinase A6 | 072133 | 183035 | 165259 | 29, 40, 28 |
| X | RNF113A | ring finger protein 113A | 125352 | 125351 | 125356 | 21, 30, 26 |
| X | RNF128 | ring finger protein 128, E3 ubiquitin protein ligase | 133135 | 147231 | 133138 | 31, 2, 34 |
| X | RLIM | ring finger protein, LIM domain interacting | 131263 | 147100 | 050030 | 31, 15, 43 |
| X | RIPPLY1 | ripply transcriptional repressor 1 | 147223 | 133131 | 165376 | 44, 19, 17 |
| X | RBM3 | RNA binding motif (RNP1, RRM) protein 3 | 102317 | 068354 | 101940 | 39, 9, 36 |
| X | RBM10 | RNA binding motif protein 10 | 182872 | 147123 | 130985 | 21, 3, 9 |
| X | RBM41 | RNA binding motif protein 41 | 089682 | 165376 | 198088 | 4, 40, 31 |
| X | RBMX | RNA binding motif protein, X-linked | 147274 | 129675 | 165370 | 39, 13, 3 |
| X | RBMX2 | RNA binding motif protein, X-linked 2 | 134597 | 147262 | 165675 | 31, 33, 11 |
| X | RBMXL3 | RNA binding motif protein, X-linked like 3 | 175718 | 130224 | 102021 | 38, |
| X | RPL36A- | RPL36A-HNRNPH2 readthrough | 257529 | 241343 | 102393 | |
| | HNRNPH2 | | | | | |
| X | S100G | S100 calcium binding protein G | 169906 | 047230 | 169895 | |
| X | SASH3 | SAM and SH3 domain containing 3 | 122122 | 122121 | 188706 | 41, 5, 21 |
| X | SAGE1 | sarcoma antigen 1 | 181433 | 269586 | 169446 | 7, 38, 8 |
| X | SEPT6 | septin 6 | 125354 | 186416 | 187808 | 39, 41, 36 |
| X | STK26 | serine/threonine kinase 26 | 134602 | 171054 | 165694 | 39, 11, 4 |
| X | SERPINA7 | serpin family A member 7 | 123561 | 123572 | 157502 | 19, 15, 20 |
| X | SCML1 | sex comb on midleg-like 1 (Drosophila) | 047634 | 188158 | 131831 | 19, 11, 38 |
| X | SCML2 | sex comb on midleg-like 2 (Drosophila) | 102098 | 177324 | 008086 | 38, 11, 40 |
| X | SH2D1A | SH2 domain containing 1A | 183918 | 101972 | 282419 | 39, 5, 7 |
| X | SH3BGRL | SH3 domain binding glutamate rich protein like | 131171 | 198157 | 196767 | 45, 7, 13 |
| X | SH3KBP1 | SH3 domain containing kinase binding protein 1 | 147010 | 180815 | 173681 | 21, 43, 18 |
| X | SHOX | short stature homeobox | 185960 | 167393 | 205755 | 14, |
| X | SHROOM2 | shroom family member 2 | 146950 | 101850 | 234469 | 30, 8, 33 |
| X | SHROOM4 | shroom family member 4 | 158352 | 274588 | 130385 | 45, 40, 37 |
| X | SSR4 | signal sequence receptor subunit 4 | 180879 | 067829 | 067840 | 29, 31, 33 |
| X | SLITRK2 | SLIT and NTRK like family member 2 | 185985 | 203923 | 221870 | 11, 18, 8 |
| X | SLITRK4 | SLIT and NTRK like family member 4 | 179542 | 189252 | 268988 | 42, 17, 8 |
| X | SMIM10 | small integral membrane protein 10 | 184785 | 101928 | 212747 | 24, 29, 35 |
| X | SMIM10L2A | small integral membrane protein 10 like 2A | 178947 | 173275 | 165359 | |
| X | SMIM10L2B | small integral membrane protein 10 like 2B | 196972 | 203950 | 224107 | 2, 17, 33 |
| X | SMIM9 | small integral membrane protein 9 | 203870 | 130830 | 185010 | 10, 38, 32 |
| X | SMPX | small muscle protein, X-linked | 091482 | 185915 | 012174 | 25, 15, 42 |
| X | SLC10A3 | solute carrier family 10 member 3 | 126903 | 102178 | 071889 | 32, 1, 3 |
| X | SLC16A2 | solute carrier family 16 member 2 | 147100 | 187969 | 131263 | 11, 31, 6 |
| X | SLC25A14 | solute carrier family 25 member 14 | 102078 | 056277 | 147262 | 34, 14, 31 |
| X | SLC25A43 | solute carrier family 25 member 43 | 077713 | 101856 | 005022 | 9, 4, 35 |
| X | SLC25A5 | solute carrier family 25 member 5 | 005022 | 077713 | 018610 | 30, |
| X | SLC25A53 | solute carrier family 25 member 53 | 269743 | 269226 | 166707 | 31, 39, 37 |
| X | SLC25A6 | solute carrier family 25 member 6 | 169100 | 185291 | 169093 | 9, 21, 1 |
| X | SLC35A2 | solute carrier family 35 member A2 | 102100 | 102103 | 102096 | 13, 3, 4 |
| X | SLC38A5 | solute carrier family 38 member 5 | 017483 | 269791 | 068438 | 24, 9, 4 |
| X | SLC6A14 | solute carrier family 6 member 14 | 268104 | 180772 | 204019 | 42, 37, 23 |
| X | SLC6A8 | solute carrier family 6 member 8 | 130821 | 130822 | 185825 | 35, 29, 16 |
| X | SLC7A3 | solute carrier family 7 member 3 | 165349 | 120498 | 147164 | 39, 21, 12 |
| X | SLC9A6 | solute carrier family 9 member A6 | 198689 | 169446 | 022267 | 29, 11, 8 |
| X | SLC9A7 | solute carrier family 9 member A7 | 065923 | 147119 | 102218 | |
| X | SNX12 | sorting nexin 12 | 147164 | 165349 | 184481 | 9, 45, 3 |
| X | SOWAHD | sosondowah ankyrin repeat domain family member D | 187808 | 125354 | 198918 | 20, 32, 19 |
| X | SPANXA2 | SPANX family member A2 | 203926 | 198021 | 196406 | 19, |
| X | SPANXB1 | SPANX family member B1 | 227234 | 184258 | 182195 | |
| X | SPANXC | SPANX family member C | 198573 | 182195 | 198021 | 38, |
| X | SPANXD | SPANX family member D | 196406 | 203926 | 165509 | 19, 38, 10 |
| X | SPANXN1 | SPANX family member N1 | 203923 | 276380 | 185985 | |
| X | SPANXN2 | SPANX family member N2 | 268988 | 179542 | 276380 | 8, |
| X | SPANXN3 | SPANX family member N3 | 189252 | 189326 | 179542 | 38, |
| X | SPANXN4 | SPANX family member N4 | 189326 | 046774 | 189252 | 38, |
| X | SPANXN5 | SPANX family member N5 | 204363 | 268447 | 171405 | 17, |
| X | SPACA5 | sperm acrosome associated 5 | 171489 | 221994 | 171478 | |
| X | SPACA5B | sperm acrosome associated 5B | 171478 | 171489 | 165583 | |
| X | SPANXA1 | sperm protein associated with the nucleus, X-linked, family member A1 | 198021 | 198573 | 203926 | 19, 7, 38 |
| X | SATL1 | spermidine/spermine N1-acetyl transferase-like 1 | 184788 | 155008 | 147180 | 9, 15, 16 |
| X | SAT1 | spermidine/spermine N1-acetyltransferase 1 | 130066 | 123130 | 184831 | 30, 5, 36 |
| X | SMS | spermine synthase | 102172 | 230797 | 102174 | 2, 11, 23 |
| X | SPIN2A | spindlin family member 2A | 147059 | 186787 | 165591 | 5, 6, 14 |
| X | SPIN2B | spindlin family member 2B | 186787 | 204271 | 147059 | |
| X | SPIN3 | spindlin family member 3 | 204271 | 204272 | 186787 | 9, 8, 42 |
| X | SPIN4 | spindlin family member 4 | 186767 | 198205 | 131089 | 11, 7, 44 |
| X | SPRY3 | sprouty RTK signaling antagonist 3 | 168939 | 185973 | 124333 | 24, 33, 7 |
| X | SRPK3 | SRSF protein kinase 3 | 184343 | 198753 | 067829 | 25, 43, 31 |
| X | SOX3 | SRY-box 3 | 134595 | 203933 | 184258 | 38, 8, 12 |
| X | SSX1 | SSX family member 1 | 126752 | 165583 | 165584 | 34, 7, 35 |
| X | SSX2 | SSX family member 2 | 241476 | 187754 | 268447 | |
| X | SSX2B | SSX family member 2B | 268447 | 241476 | 204363 | 7, 35, 19 |
| X | SSX3 | SSX family member 3 | 165584 | 126752 | 268009 | 7, 38, 35 |
| X | SSX4 | SSX family member 4 | 268009 | 165584 | 269791 | |
| X | SSX4B | SSX family member 4B | 269791 | 268009 | 017483 | |
| X | SSX5 | SSX family member 5 | 165583 | 171478 | 126752 | 35, |
| X | SSX7 | SSX family member 7 | 187754 | 204382 | 241476 | |
| X | STARD8 | StAR related lipid transfer domain containing 8 | 130052 | 181704 | 090776 | 26, 32, 6 |
| X | STS | steroid sulfatase (microsomal), isozyme S | 101846 | 130021 | 182583 | 32, 18, 1 |
| X | STAG2 | stromal antigen 2 | 101972 | 101966 | 183918 | 11, 4, 22 |
| X | SMC1A | structural maintenance of chromosomes 1A | 072501 | 124313 | 158423 | 22, 19, 4 |
| X | SUV39H1 | suppressor of variegation 3-9 homolog 1 | 101945 | 015285 | 171433 | 21, 39, 24 |
| X | SRPX | sushi repeat containing protein, X-linked | 101955 | 147041 | 156313 | 45, 15, 43 |
| X | SRPX2 | sushi repeat containing protein, X-linked 2 | 102359 | 3 | 102362 | 45, 43, 26 |
| X | SMARCA1 | SWI/SNF related, matrix associated, actin dependent regulator of chromatin, | 102038 | 123165 | 122126 | 11, 12, 27 |
| | | subfamily a, member 1 | | | | |
| X | SYAP1 | synapse associated protein 1 | 169895 | 169906 | 086712 | 19, 2, 29 |
| X | SYN1 | synapsin I | 008056 | 078061 | 102265 | 26, 8, 28 |
| X | SYP | synaptophysin | 102003 | 012211 | 102001 | 8, 14, 26 |
| X | SYTL4 | synaptotagmin like 4 | 102362 | 102359 | 101811 | 44, 13, 1 |
| X | SYTL5 | synaptotagmin like 5 | 147041 | 187516 | 101955 | 11, 24, 42 |
| X | TAZ | tafazzin | 102125 | 013563 | 071553 | 31, 9, 5 |
| X | TAF1 | TATA-box binding protein associated factor 1 | 147133 | 147166 | 147162 | 18, 4, 40 |
| X | TAF7L | TATA-box binding protein associated factor 7 like | 102387 | 102385 | 126953 | 38, 19, 14 |
| X | TAF9B | TATA-box binding protein associated factor 9b | 187325 | 102144 | 173198 | 31, 4, 42 |
| X | TXLNG | taxilin gamma | 086712 | 169895 | 102054 | 1, 3, 6 |
| X | TBC1D25 | TBC1 domain family member 25 | 068354 | 147155 | 102317 | 34, 32, 21 |
| X | TBC1D8B | TBC1 domain family member 8B | 133138 | 133135 | 133131 | 31, 45, 23 |
| X | TBX22 | T-box22 | 122145 | 078596 | 174016 | 38, 35, |
| X | TCP11X2 | t-complex 11 family, X-linked 2 | 215029 | 269437 | 158164 | |
| X | TENM1 | teneurin transmembrane protein 1 | 009694 | 282419 | 282815 | 40, 39, 26 |
| X | TNMD | tenomodulin | 5 | 165194 | 3 | 15, 26, 33 |
| X | TEX11 | testis expressed 11 | 120498 | 082458 | 165349 | 10, 38, 36 |
| X | TEX13A | testis expressed 13A | 268629 | 189108 | 123572 | 38, 8, |
| X | TEX13B | testis expressed 13B | 170925 | 080561 | 101842 | |
| X | TEX28 | testis expressed 28 | 278057 | 269433 | 007350 | 38, 8, |
| X | TSPAN6 | tetraspanin 6 | 3 | 5 | 102359 | 42, 43, 4 |
| X | TSPAN7 | tetraspanin 7 | 156298 | 036473 | 165175 | 8, 2, 11 |
| X | TEX13C | TEX13 family member C | 282815 | 009694 | 198354 | |
| X | TEX13D | TEX13 family member D | 282419 | 183918 | 009694 | |
| X | TGIF2LX | TGFB induced factor homeobox 2 like, X-linked | 153779 | 147183 | 174740 | 38, |
| X | TAB3 | TGF-beta activated kinase 1/MAP3K7 binding protein 3 | 157625 | 198814 | 132446 | 11, 40, 26 |
| X | THOC2 | THO complex 2 | 125676 | 125675 | 101966 | 22, 4, 11 |
| X | TREX2 | three prime repair exonuclease 2 | 183479 | 189420 | 213397 | 17, 38, |
| X | TMSB15A | thymosin beta 15a | 158164 | 215029 | 125962 | 29, 11, 15 |
| X | TMSB15B | thymosin beta 15B | 158427 | 123570 | 123569 | 19, 39, 8 |
| X | TMSB4X | thymosin beta 4, X-linked | 205542 | 101916 | 187268 | |
| X | TIMP1 | TIMP metallopeptidase inhibitor 1 | 102265 | 008056 | 126759 | 18, 43, 6 |
| X | TLR7 | toll like receptor 7 | 196664 | 101911 | 101916 | 29, 10, 22 |
| X | TLR8 | toll like receptor 8 | 101916 | 196664 | 205542 | 10, 5, 32 |
| X | TRAPPC2 | trafficking protein particle complex 2 | 196459 | 123595 | 046651 | 39, 9, 22 |
| X | TCEAL1 | transcription elongation factor A like 1 | 172465 | 196507 | 123562 | 11, 31, 1 |
| X | TCEAL2 | transcription elongation factor A like 2 | 184905 | 166432 | 204071 | 1, 31, 2 |
| X | TCEAL3 | transcription elongation factor A like 3 | 196507 | 133142 | 172465 | 43, 26, 30 |
| X | TCEAL4 | transcription elongation factor A like 4 | 133142 | 172476 | 196507 | 4, 7, 19 |
| X | TCEAL5 | transcription elongation factor A like 5 | 204065 | 180964 | 133134 | 14, 32, 8 |
| X | TCEAL6 | transcription elongation factor A like 6 | 204071 | 184905 | 184515 | 2, 14, 38 |
| X | TCEAL7 | transcription elongation factor A like 7 | 182916 | 133134 | 185222 | 26, 31, 45 |
| X | TCEAL8 | transcription elongation factor A like 8 | 180964 | 102409 | 204065 | 31, 4, 29 |
| X | TCEAL9 | transcription elongation factor A like 9 | 185222 | 182916 | 166681 | |
| X | TCEANC | transcription elongation factor A N-terminal and central domain containing | 176896 | 198759 | 123595 | |
| X | TFE3 | transcription factor binding to IGHM enhancer 3 | 068323 | 068400 | 147144 | 26, 18, 35 |
| X | TFDP3 | transcription factor Dp family member 3 | 183434 | 134588 | 076716 | 38, 35, |
| X | TBL1X | transducin beta like 1X-linked | 101849 | 177138 | 101850 | 22, 29, 15 |
| X | TRPC5 | transient receptor potential cation channel subfamily C member 5 | 072315 | 101901 | 204025 | 40, 32, 8 |
| X | TKTL1 | transketolase like 1 | 007350 | 278057 | 196924 | 38, 7, 30 |
| X | TIMM17B | translocase of inner mitochondrial membrane 17 homolog B (yeast) | 126768 | 102109 | 102103 | 31, 34, 20 |
| X | TIMM8A | translocase of inner mitochondrial membrane 8 homolog A (yeast) | 126953 | 102387 | 010671 | 7, 34, 4 |
| X | TMEM164 | transmembrane protein 164 | 157600 | 068366 | 101935 | 43, 22, 1 |
| X | TMEM185A | transmembrane protein 185A | 269556 | 268738 | 185247 | 26, 13, 31 |
| X | TMEM187 | transmembrane protein 187 | 177854 | 172534 | 184216 | 22, 33, 5 |
| X | TMEM255A | transmembrane protein 255A | 125355 | 177485 | 101892 | 26, 39, 38 |
| X | TMEM257 | transmembrane protein 257 | 221870 | 185985 | 235699 | 14, 8, 20 |
| X | TMEM27 | transmembrane protein 27 | 147003 | 130234 | 169239 | 17, 10, 19 |
| X | TMEM31 | transmembrane protein 31 | 179363 | 188828 | 123560 | 38, 32, |
| X | TMEM35A | transmembrane protein 35A | 126950 | 188917 | 102384 | 8, 29, 26 |
| X | TMEM47 | transmembrane protein 47 | 147027 | 185448 | 189132 | 45, 11, 36 |
| X | TMLHE | trimethyllysine hydroxylase, epsilon | 185973 | 277745 | 168939 | 29, 21, 7 |
| X | TRMT2B | tRNA methyltransferase 2 homolog B | 188917 | 174225 | 126950 | 21, 9, 29 |
| X | TRO | trophinin | 067445 | 102316 | 158571 | 39, 8, 38 |
| X | TRPC5OS | TRPC5 opposite strand | 204025 | 072315 | 187823 | |
| X | TSC22D3 | TSC22 domain family member 3 | 157514 | 147224 | 170935 | 2, 10, 1 |
| X | TSPYL2 | TSPY like 2 | 184205 | 184194 | 126012 | 26, 5, 31 |
| X | TSR2 | TSR2, ribosome maturation factor | 158526 | 196632 | 102302 | 9, 3, 27 |
| X | UBQLN2 | ubiquilin 2 | 188021 | 102349 | 204272 | 20, 33, 23 |
| X | UBE2A | ubiquitin conjugating enzyme E2 A | 077721 | 018610 | 186416 | 43, 13, 32 |
| X | UBE2NL | ubiquitin conjugating enzyme E2 N like (gene/pseudogene) | 276380 | 268988 | 203923 | 36, 2, 14 |
| X | UBL4A | ubiquitin like 4A | 102178 | 196976 | 126903 | 3, 2, 9 |
| X | UBA1 | ubiquitin like modifier activating enzyme 1 | 130985 | 182872 | 102225 | 21, 12, 27 |
| X | USP11 | ubiquitin specific peptidase 11 | 102226 | 102225 | 147117 | 8, 39, 38 |
| X | USP26 | ubiquitin specific peptidase 26 | 134588 | 171004 | 183434 | 6, 38, |
| X | USP27X | ubiquitin specific peptidase 27, X-linked | 273820 | 101951 | 171365 | 20, 16, 17 |
| X | USP51 | ubiquitin specific peptidase 51 | 247746 | 187601 | 189299 | 26, 15, 22 |
| X | USP9X | ubiquitin specific peptidase 9, X-linked | 124486 | 180182 | 215301 | 20, 3, 31 |
| X | UXT | ubiquitously expressed prefoldin like chaperone | 126756 | 126767 | 279988 | 30, 7, 3 |
| X | UPF3B | UPF3 regulator of nonsense transcripts homolog B (yeast) | 125351 | 198918 | 125352 | 18, 25, 6 |
| X | UPRT | uracil phosphoribosyltransferase homolog | 094841 | 131269 | 102383 | 2, 43, 37 |
| X | UTP14A | UTP14A small subunit processome component | 156697 | 188706 | 085185 | 41, 13, 3 |
| X | VCX2 | variable charge, X-linked | 182583 | 101846 | 006757 | 38, |
| X | VCX2 | variable charge, X-linked 2 | 177504 | 006757 | 205642 | 38, |
| X | VCX3A | variable charge, X-linked 3A | 169059 | 146938 | 130021 | 37, 35, 38 |
| X | VCX3B | variable charge, X-linked 3B | 205642 | 177504 | 011201 | 38, |
| X | VEGFD | vascular endothelial growth factor D | 165197 | 165195 | 087842 | 1, 20, 2 |
| X | VAMP7 | vesicle associated membrane protein 7 | 124333 | 168939 | 124334 | 35, 29, 45 |
| X | VGLL1 | vestigial like family member 1 | 102243 | 102241 | 102245 | 32, 23, 34 |
| X | VBP1 | VHL binding protein 1 | 155959 | 185515 | 155961 | 34, 7, 29 |
| X | VMA21 | VMA21 vacuolar H+-ATPase homolog (S. cerevisiae) | 160131 | 102195 | 166049 | 22, 11, 35 |
| X | VSIG1 | V-set and immunoglobulin domain containing 1 | 101842 | 170925 | 101843 | 38, 37, 30 |
| X | VSIG4 | V-set and immunoglobulin domain containing 4 | 155659 | 147065 | 089472 | 10, 32, 1 |
| X | WDR13 | WD repeat domain 13 | 101940 | 102317 | 015285 | 3, 34, 9 |
| X | WDR44 | WD repeat domain 44 | 131725 | 003096 | 147251 | 24, 42, 22 |
| X | WDR45 | WD repeat domain 45 | 196998 | 243279 | 068394 | 29, 18, 9 |
| X | WAS | Wiskott-Aldrich syndrome | 015285 | 101940 | 101945 | 21, 41, 5 |
| X | WNK3 | WNK lysine deficient protein kinase 3 | 196632 | 184083 | 158526 | 31, 11, 38 |
| X | WWC3 | WWC family member 3 | 047644 | 234469 | 073464 | 29, 22, 21 |
| X | XAGE1A | X antigen family member 1A | 204379 | 155622 | 204382 | 10, |
| X | XAGE1B | X antigen family member 1B | 204382 | 204379 | 187754 | 10, |
| X | XAGE2 | X antigen family member 2 | 155622 | 154545 | 204379 | 32, 15, 6 |
| X | XAGE3 | X antigen family member 3 | 171402 | 171405 | 179304 | 32, |
| X | XAGE5 | X antigen family member 5 | 171405 | 204363 | 171402 | 38, |
| X | XG | Xg blood group | 124343 | 002586 | 056998 | 1, 26, 24 |
| X | XKRX | XK related, X-linked | 182489 | 007952 | 174225 | 22, 24, 39 |
| X | XIAP | X-linked inhibitor of apoptosis | 101966 | 125676 | 101972 | 13, 22, 26 |
| X | XK | X-linked Kx blood group | 047597 | 147036 | 165168 | 26, 19, 25 |
| X | XPNPEP2 | X-prolyl aminopeptidase 2 | 122121 | 171388 | 122122 | 15, 17, 40 |
| X | YIPF6 | Yip1 domain family member 6 | 181704 | 079482 | 130052 | 31, 29, 32 |
| X | YY2 | YY2 transcription factor | 230797 | 012174 | 102172 | 36, 38, 20 |
| X | ZFP92 | ZFP92 zinc finger protein | 189420 | 214897 | 183479 | |
| X | ZIC3 | Zic family member 3 | 156925 | 165370 | 129682 | 12, 38, 40 |
| X | ZBTB33 | zinc finger and BTB domain containing 33 | 177485 | 131721 | 125355 | 43, 11, 7 |
| X | ZBED1 | zinc finger BED-type containing 1 | 214717 | 169084 | 002586 | 34, 28, 1 |
| X | ZC4H2 | zinc finger C4H2-type containing | 126970 | 102043 | 102053 | 39, 21, 38 |
| X | ZC3H12B | zinc finger CCCH-type containing 12B | 102053 | 126970 | 001497 | 36, 39, 25 |
| X | ZRSR2 | zinc finger CCCH-type, RNA binding motif and serine/arginine rich 2 | 169249 | 169239 | 182287 | 41, 19, 22 |
| X | ZCCHC12 | zinc finger CCHC-type containing 12 | 174460 | 131724 | 175556 | 8, 31, 9 |
| X | ZCCHC13 | zinc finger CCHC-type containing 13 | 187969 | 204116 | 147100 | 38, |
| X | ZCCHC16 | zinc finger CCHC-type containing 16 | 187823 | 204025 | 182508 | 17, |
| X | ZCCHC18 | zinc finger CCHC-type containing 18 | 166707 | 269743 | 123575 | 15, 20, 28 |
| X | ZCCHC5 | zinc finger CCHC-type containing 5 | 179300 | 173198 | 147145 | 15, 45, 17 |
| X | ZDHHC15 | zinc finger DHHC-type containing 15 | 102383 | 094841 | 186675 | 11, 42, 31 |
| X | ZDHHC9 | zinc finger DHHC-type containing 9 | 188706 | 122122 | 156697 | 17, 8, 13 |
| X | ZMAT1 | zinc finger matrin-type 1 | 166432 | 126952 | 184905 | 31, 14, 22 |
| X | ZMYM3 | zinc finger MYM-type containing 3 | 147130 | 169562 | 147140 | 27, 43, 12 |
| X | ZNF157 | zinc finger protein 157 | 147117 | 102226 | 147124 | 45, |
| X | ZNF182 | zinc finger protein 182 | 147118 | 197779 | 221994 | 42, 39, 22 |
| X | ZNF185 | zinc finger protein 185 (LIM domain) | 147394 | 147383 | 198883 | 7, 31, 9 |
| X | ZNF275 | zinc finger protein 275 | 063587 | 225110 | 214897 | |
| X | ZNF280C | zinc finger protein 280C | 056277 | 134594 | 102078 | 24, 37, 12 |
| X | ZNF41 | zinc finger protein 41 | 147124 | 147117 | 078061 | 42, 22, 25 |
| X | ZNF449 | zinc finger protein 449 | 173275 | 283644 | 178947 | 1, 24, 32 |
| X | ZNF630 | zinc finger protein 630 | 221994 | 147118 | 171489 | 39, 8, 6 |
| X | ZNF645 | zinc finger protein 645 | 175809 | 102174 | 184735 | 38, |
| X | ZNF674 | zinc finger protein 674 | 251192 | 147121 | 147119 | 9, 42, 23 |
| X | ZNF711 | zinc finger protein 711 | 147180 | 184788 | 124429 | 45, 39, 25 |
| X | ZNF75D | zinc finger protein 75D | 186376 | 169551 | 238210 | |
| X | ZNF81 | zinc finger protein 81 | 197779 | 279988 | 147118 | 42, 17, 27 |
| X | ZFX | zinc finger protein, X-linked | 005889 | 130741 | 067992 | 13, 7, 43 |
| X | ZXDA | zinc finger, X-linked, duplicated A | 198205 | 215174 | 186767 | 3, 40, 42 |
| X | ZXDB | zinc finger, X-linked, duplicated B | 198455 | 165591 | 215174 | 43, 1, 19 |
| Y | AMELY | amelogenin, Y-linked | 099721 | 168757 | 092377 | 10, |
| Y | BPY2 | basic charge, Y-linked, 2 | 183753 | 169789 | 188120 | |
| Y | BPY2B | basic charge, Y-linked, 2B | 183795 | 279274 | 187191 | |
| Y | BPY2C | basic charge, Y-linked, 2C | 185894 | 205916 | 279115 | |
| Y | CDY1 | chromodomain Y-linked 1 | 172288 | 280301 | 233803 | |
| Y | CDY1B | chromodomain Y-linked 1B | 172352 | 205944 | 279287 | 38, |
| Y | CDY2A | chromodomain Y-linked 2A | 182415 | 129873 | 172468 | |
| Y | CDY2B | chromodomain Y-linked 2B | 129873 | 165246 | 182415 | |
| Y | DDX3Y | DEAD-box helicase 3, Y-linked | 067048 | 114374 | 183878 | 42, 39, 36 |
| Y | DAZ1 | deleted in azoospermia 1 | 188120 | 183753 | 205944 | 37, 38, 20 |
| Y | DAZ2 | deleted in azoospermia 2 | 205944 | 188120 | 172352 | 37, 38, |
| Y | DAZ3 | deleted in azoospermia 3 | 187191 | 183795 | 205916 | |
| Y | DAZ4 | deleted in azoospermia 4 | 205916 | 187191 | 185894 | |
| Y | EIF1AY | eukaryotic translation initiation factor 1A, Y-linked | 198692 | 012817 | 280969 | 29, 5, 17 |
| Y | HSFY1 | heat shock transcription factor, Y-linked 1 | 172468 | 182415 | 169953 | |
| Y | HSFY2 | heat shock transcription factor, Y-linked 2 | 169953 | 172468 | 279335 | |
| Y | KDM5D | lysine demethylase 5D | 012817 | 279335 | 198692 | 39, 1, 43 |
| Y | NLGN4Y | neuroligin 4, Y-linked | 165246 | 129862 | 129873 | 43, 26, 31 |
| Y | PRORY | proline rich, Y-linked | 183146 | 280969 | 242875 | |
| Y | PCDH11Y | protocadherin 11 Y-linked | 099715 | 176679 | 168757 | 6, 38, 8 |
| Y | PRY | PTPN13-like, Y-linked | 169789 | 226941 | 183753 | |
| Y | PRY2 | PTPN13-like, Y-linked 2 | 169807 | 242389 | 169800 | |
| Y | RPS4Y1 | ribosomal protein S4, Y-linked 1 | 129824 | 184895 | 067646 | 37, 29, 11 |
| Y | RPS4Y2 | ribosomal protein S4, Y-linked 2 | 280969 | 198692 | 183146 | |
| Y | RBMY1A1 | RNA binding motif protein, Y-linked, family 1, member A1 | 234414 | 242875 | 244395 | 38, |
| Y | RBMY1B | RNA binding motif protein, Y-linked, family 1, member B | 242875 | 183146 | 234414 | 38, 35, 19 |
| Y | RBMY1D | RNA binding motif protein, Y-linked, family 1, member D | 244395 | 234414 | 242389 | |
| Y | RBMY1E | RNA binding motif protein, Y-linked, family 1, member E | 242389 | 244395 | 169807 | 38, 35, 19 |
| Y | RBMY1F | RNA binding motif protein, Y-linked, family 1, member F | 169800 | 169807 | 226941 | 38, |
| Y | RBMY1J | RNA binding motif protein, Y-linked, family 1, member J | 226941 | 169800 | 169789 | |
| Y | SRY | sex determining region Y | 184895 | 233803 | 129824 | |
| Y | TSPY1 | testis specific protein, Y-linked 1 | 258992 | 228927 | 236424 | |
| Y | TSPY10 | testis specific protein, Y-linked 10 | 236424 | 258992 | 114374 | |
| Y | TSPY2 | testis specific protein, Y-linked 2 | 168757 | 099715 | 099721 | 38, 8, |
| Y | TSPY3 | testis specific protein, Y-linked 3 | 228927 | 229549 | 258992 | 38, 8, |
| Y | TSPY4 | testis specific protein, Y-linked 4 | 233803 | 279547 | 229549 | |
| Y | TSPY8 | testis specific protein, Y-linked 8 | 229549 | 233803 | 228927 | |
| Y | TGIF2LY | TGFB induced factor homeobox 2 like, Y-linked | 176679 | 067646 | 099715 | 38, |
| Y | TMSB4Y | thymosin beta 4, Y-linked | 154620 | 183878 | 129864 | 31, 26, 38 |
| Y | TBL1Y | transducin beta like 1, Y-linked | 092377 | 099721 | 279435 | 33, 38, 12 |
| Y | USP9Y | ubiquitin specific peptidase 9, Y-linked | 114374 | 236424 | 067048 | 26, 39, 43 |
| Y | UTY | ubiquitously transcribed tetratricopeptide repeat containing, Y-linked | 183878 | 067048 | 154620 | 42, 13, 31 |
| Y | VCY | variable charge, Y-linked | 129864 | 154620 | 129862 | |
| Y | VCY1B | variable charge, Y-linked 1B | 129862 | 129864 | 165246 | |
| Y | ZFY | zinc finger protein, Y-linked | 067646 | 129824 | 176679 | 4, 26, 17 |

Table 10 provides a listing of tissues and their designated codes for expression in Tables 1-9. These tissues may embody the target tissue of an embodiment of the invention. In some embodiments, more than one tissue may be targeted. In some embodiments, different cell types of any one or more of these tissues may be targeted. In some embodiments, the same one or more genes may be found to be expressed in one or more of the tissues listed. In some embodiments, any one or more of the listed tissues may be perturbed by a stimulus or may exhibit an outcome attendant to such perturbation.

TABLE 10
Tissue Expression Code

| Tissue | Code |
|---|---|
| adipose tissue | 1 |
| adrenal gland | 2 |
| ascites | 3 |
| bladder | 4 |
| blood | 5 |
| bone | 6 |
| bone marrow | 7 |
| brain | 8 |
| cervix | 9 |
| connective tissue | 10 |
| ear | 11 |
| embryonic tissue | 12 |
| esophagus | 13 |
| eye | 14 |
| heart | 15 |
| intestine | 16 |
| kidney | 17 |
| larynx | 18 |
| liver | 19 |
| lung | 20 |
| lymph | 21 |
| lymph node | 22 |
| mammary gland | 23 |
| mouth | 24 |
| muscle | 25 |
| nerve | 26 |
| ovary | 27 |
| pancreas | 28 |
| parathyroid | 29 |
| pharynx | 30 |
| pituitary gland | 31 |
| placenta | 32 |
| prostate | 33 |
| salivary gland | 34 |
| skin | 35 |
| spleen | 36 |
| stomach | 37 |
| testis | 38 |
| thymus | 39 |
| thyroid | 40 |
| tonsil | 41 |
| trachea | 42 |
| umbilical cord | 43 |
| uterus | 44 |
| vascular | 45 |

### Topologically Associating Domains (TADs)

Topologically Associating Domains (TADs), alternatively known as topological domains, are hierarchical units that are subunits of the mammalian chromosome structure. See, Dixon et al., Nature, 485(7398):376-80, 2012; Filippova et al., Algorithms for Molecular Biology, 9:14, 2014; Gibcus and Dekker Molecular Cell, 49(5):773-82, 2013; Naumova et al., Science, 42(6161):948-53, 2013; which are hereby incorporated by reference in their entireties. TADs are megabase-sized chromosomal regions that demarcate a microenvironment that allows genes and regulatory elements to make productive DNA-DNA contacts. TADs are defined by DNA-DNA interaction frequencies. The boundaries of TADs consist of regions where relatively fewer DNA-DNA interactions occur, as described in Dixon et al., Nature, 485(7398):376-80, 2012; Nora et al., Nature, 485(7398):381-5, 2012; which are hereby incorporated by reference in their entireties. TADs represent structural chromosomal units that function as gene expression regulators.

TADs may contain about 7 or more protein-coding genes and have boundaries that are shared by the different cell types. See, Smallwood et al., Current Opinion in Cell Biology, 25(3):387-94, 2013, which is hereby incorporated by reference in its entirety. Some TADs contain active genes and others contain repressed genes, as the expression of genes within a single TAD is usually correlated. See, Cavalli et al., Nature Structural & Molecular Biology, 20(3):290-9, 2013, which is hereby incorporated by reference in its entirety. Sequences within a TAD find each other with high frequency and have concerted, TAD-wide histone chromatin signatures, expression levels, DNA replication timing, lamina association, and chromocenter association. See, Dixon et al., Nature, 485(7398):376-80, 2012; Le Dily et al., Genes Development, 28:2151-62, 2014; Dixon et al., Nature, 485(7398):376-80, 2012; Wijchers, Genome Research, 25:958-69, 2015, which are hereby incorporated by reference in their entireties.

Gene loops and other structures within TADs influence the activities of transcription factors (TFs), cohesin, and 11-zinc finger protein (CTCF), a transcriptional repressor. See, Baranello et al., Proceedings of the National Academy of Sciences, 111(3):889-9, 2014, which is hereby incorporated by reference in its entirety. The structures within TADs include cohesin-associated enhancer-promoter loops that are produced when enhancer-bound TFs bind cofactors, for example Mediator, that, in turn, bind RNA polymerase II at promoter sites. See, Lee and Young, Cell, 152(6):1237-51, 2013; Lelli et al., 2012; Roeder, Annual Reviews Genetics 46:43-68, 2005; Spitz and Furlong, Nature Reviews Genetics, 13(9):613-26, 2012; Dowen et al., Cell, 159(2): 374-387, 2014; Lelli et al., Annual Review of Genetics, 46:43-68, 2012, which are hereby incorporated by reference in their entireties. The cohesin-loading factor Nipped-B-like protein (NIPBL) binds Mediator and loads cohesin at these enhancer-promoter loops. See, Kagey et al., Nature, 467(7314):430-5, 2010, which is hereby incorporated by reference in its entirety.

TADs have similar boundaries in all human cell types examined and constrain enhancer-gene interactions. See, Dixon et al., Nature, 518:331-336, 2015; Dixon et al., Nature, 485:376-380, 2012, which are hereby incorporated by reference in their entireties. This architecture of the genome helps explain why most DNA contacts occur within the TADs and enhancer-gene interactions rarely occur between chromosomes. However, TADs provide only partial insight into the molecular mechanisms that influence specific enhancer-gene interactions within TADs.

Long-range genomic contacts segregate TADs into an active and inactive compartment. See, Lieberman-Aden et al., Science, 326:289-93, 2009, which is hereby incorporated by reference in its entirety. The loops formed between TAD boundaries seem to represent the longest-range contacts that are stably and reproducibly formed between specific pairs of sequences. See, Dixon et al., Nature, 485(7398):376-80, 2012, which is hereby incorporated by reference in its entirety.

In some embodiments, the methods of the present invention are used to after gene expression from genes located in a TAD. In some embodiments, TAD regions are modified to alter gene expression of a non-canonical pathway as defined herein or as definable using the methods described herein.

Insulated Neighborhoods

As used herein, an “insulated neighborhood” or “IN” is defined as a chromosome structure formed by the looping of two interacting sites in the chromosome sequence. These interacting sites may comprise CCCTC-Binding factor (CTCF). These CTCF sites are often co-occupied by cohesin. The integrity of these cohesin-associated chromosome structures affects the expression of genes in the IN as well as those genes in the vicinity of the INs. A “neighborhood gene” is a gene localized within an IN. Neighborhood genes may be coding or non-coding. Such neighborhood genes may be selected from those in Tables 1-9.

IN architecture is defined by at least two boundaries which come together, directly or indirectly, to form a DNA loop. The boundaries of any IN comprise a primary upstream boundary and a primary downstream boundary. Such boundaries are the outermost boundaries of any N. Within any IN loop, however, secondary loops may be formed. Such secondary loops, when present, are defined by secondary upstream boundaries and secondary downstream boundaries, relative to the primary IN. Where a primary insulated neighborhood contains more than one internal loop, the loops are numbered relative to the primary upstream boundary of the primary loop, e.g., the secondary loop (first loop within the primary loop), the tertiary loop (second loop within the primary loop), the quaternary loop (the third loop within the primary loop) and so on.

INs may be located within TADs and other gene loops. Largest insulated neighborhoods may be TADs. TADs are defined by DNA-DNA interaction frequencies, and average 0.8 Mb, contain approximately 7 protein-coding genes and have boundaries that are shared by the different cell types of an organism. According to Dowen, the expression of genes within a TAD is somewhat correlated, and thus some TADs tend to have active genes and others tend to have repressed genes. See, Dowen, et al Cell. 2014 Oct. 9; 159(2): 374-387, which is herby incorporated by reference herein in its entirety.

INs may exist as contiguous entities along a chromosome or may be separated by non-insulated neighborhood sequence regions. INs may overlap linearly only to be defined once the DNA looping regions have been joined. While INs may comprise 3-12 genes, they may contain, 1, 2, 3, 4, 5, 6, 7, 8, 9, 10, 11, 12, 13 or more genes.

A “minimal insulated neighborhood” is an insulated neighborhood having at least one neighborhood gene and associated regulatory sequence region or regions (RSRs) which facilitate the expression or repression of the neighborhood gene such as a promoter and/or enhancer and/or repressor region, and the like. It is contemplated that in some instances RSRs may coincide or even overlap with an IN boundary. RSRs, as used herein, include but are not limited to regions, sections, sites or zones along a chromosome whereby interactions with signaling molecules/signaling proteins occur in order to alter expression of a neighborhood gene. As used herein, a “signaling molecule” or a “signaling protein” is any entity, whether protein, nucleic acid (DNA or RNA), organic small molecule, lipid, sugar or other biomolecule, which interacts directly, or indirectly, with a regulatory sequence region (RSR) on a chromosome. RSR may also refer to a portion of DNA that functions as a binding site for a GSC.

One category of specialized signaling molecules/signaling proteins are transcription factors. “Transcription factors” are those signaling molecules/signaling proteins which alter, whether to increase or decrease, the transcription of a target gene, e.g., a neighborhood gene.

According to the present invention, neighborhood genes may have any number of upstream or downstream genes along the chromosome. Within any IN, there may be one or more, e.g., one, two, three, four or more, upstream and/or downstream neighborhood genes relative to the primary neighborhood gene. A “primary neighborhood gene” is a gene which is most commonly found within a specific IN along a chromosome. An upstream neighborhood gene of a primary neighborhood gene may be located within the same IN as the primary neighborhood gene. A downstream neighborhood gene of a primary neighborhood gene may be located within the same IN as the primary neighborhood gene.

The present invention provides methods of altering the penetrance of a gene or gene variant. As used herein, “penetrance” is the proportion of individuals carrying a particular variant of a gene (e.g., mutation, allele or generally a genotype, whether wild type or not) that also exhibits an associated trait (phenotype) of that variant gene. In some situations of disease, penetrance of a disease-causing mutation measured as the proportion of individuals with the mutation who exhibit clinical symptoms. Consequently, penetrance of any gene or gene variant exists on a continuum.

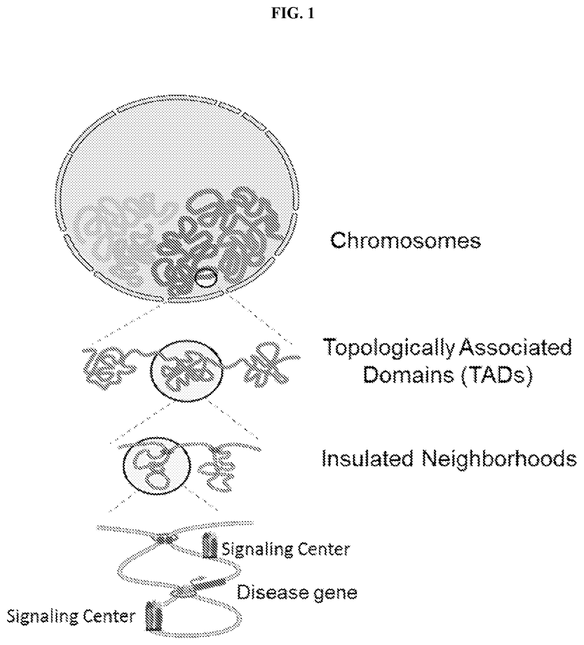

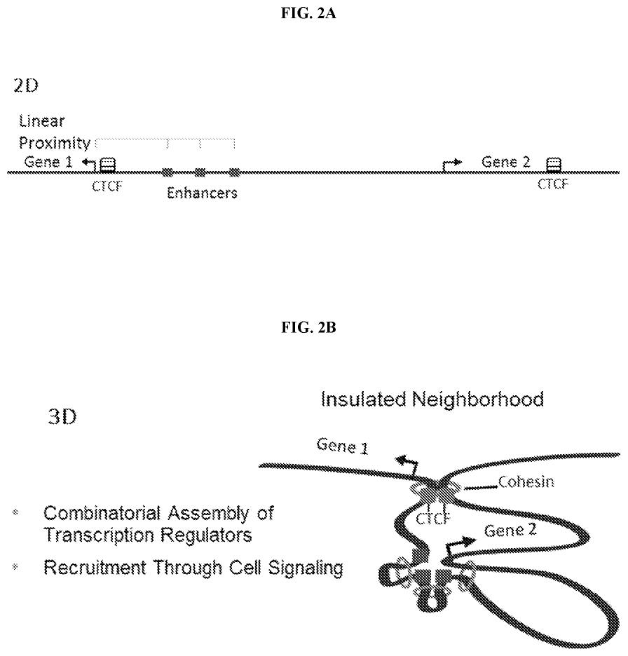

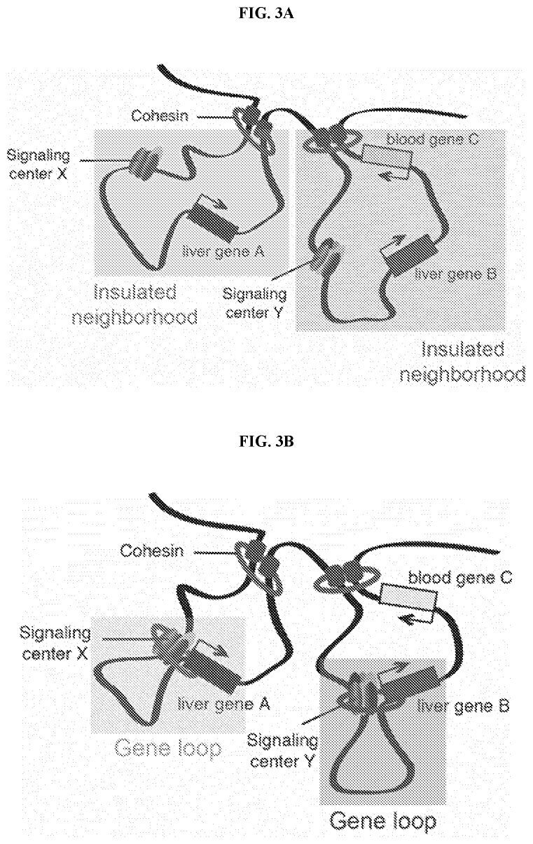

INs are functional units that may group genes under the same control mechanism, which are described in Dowen et al., Cell, 159: 374-387 (2014), which is hereby incorporated by reference in its entirety. INs provide the mechanistic background for higher-order chromosome structures, such as TADs which are shown in FIG. 1. INs are chromosome structures formed by the looping of the two interacting CTCF sites co-occupied by cohesin which are shown in FIG. 2B. The integrity of these structures is important for proper expression of local genes. Generally, 1 to 10 genes are clustered in each neighborhood with a median number of 3 genes within each one. The genes controlled by the same IN are not readily apparent from a two-dimensional view of DNA. In humans, there are about 13,801 IN in a size range of 25 kb-940 kb with a median size of 186 kb. INs are conserved among different cell types. Smaller INs that occur within a bigger IN are referred to as nested insulated neighborhoods (NINs). TADs can consist of a single IN, or one IN and one NIN and two NINs as shown in FIG. 3B.

As used herein, the term “boundary” refers to a point, limit, or range indicating where a feature, element, or property ends or begins. Accordingly, an “insulated neighborhood boundary” refers to a boundary that delimits an IN on a chromosome. According to the present invention, an IN is defined by at least two IN boundaries, a primary upstream boundary and a primary downstream boundary. The “primary upstream boundary” refers to the IN boundary located upstream of a primary neighborhood gene. The “primary downstream boundary” refers to the IN boundary located downstream of a primary neighborhood gene. Similarly, when secondary loops are present, they are defined by secondary upstream and downstream boundaries. A “secondary upstream boundary” is the upstream boundary of a secondary loop within a primary IN, and a “secondary downstream boundary” is the downstream boundary of a secondary loop within a primary N. The directionality of the secondary boundaries follows that of the primary boundaries.

Components of an IN boundary may comprise the DNA sequences at the anchor regions and associated factors (e.g., CTCF, cohesin) that facilitate the looping of the two boundaries. The DNA sequences at the anchor regions may contain at least one CTCF binding site. Experiments using the ChIP-exo technique revealed a 52 bp CTCF binding motif containing four CTCF binding modules (Ong and Corces, Nature reviews Genetics, 12:283-293, 2011, which is incorporated herein by reference in its entirety). The DNA sequences at the IN boundaries may contain insulators. In some cases, IN boundaries may also coincide or overlap with regulatory sequence regions, such as enhancer-promoter interaction sites.

In some embodiments of the present invention, disrupting or altering an insulated neighborhood boundary may be accomplished by altering specific DNA sequences (e.g., CTCF binding sites) at the boundaries. For example, existing CTCF binding sites at IN boundaries may be deleted, mutated, or inverted. Alternatively, new CTCF binding sites may be introduced to form new INs. In other embodiments, disrupting or altering an IN boundary may be accomplished by altering the histone modification (e.g., methylation, demethylation) at the boundaries. In other embodiments, disrupting or altering an IN boundary may be accomplished by altering (e.g., block) the binding of CTCF and/or cohesin to the boundaries. In cases where IN boundaries coincide or overlap with regulatory sequence regions, disrupting or altering an IN boundary may be accomplished by altering the RSR or the binding of the RSR-associated signaling molecules.

### Regulatory Elements

### Enhancers

Enhancers are gene regulatory elements that control cell type specific gene expression programs in humans. See, Buecker and Wysocka, Trends in genetics: TIG 28, 276-284, 2012; Heinz et al., Nature reviews Molecular Cell Biology, 16:144-154, 2015; Levine et al., Cell, 157:13-25, 2014; Ong and Corces, Nature reviews Genetics, 12:283-293, 2011; Ren and Yue, Cold Spring Harbor symposia on quantitative biology, 80:17-26, 2015, which are hereby incorporated by reference in their entireties. Enhancers are segments of DNA that are generally a few hundred base pairs in length that may be occupied by multiple transcription factors that recruit co-activators and RNA polymerase II to target genes. See, Bulger and Groudine, Cell, 144:327-339, 2011; Spitz and Furlong, Nature reviews Genetics, 13:613-626, 2012; Tjian and Maniatis, Cell, 77:5-8, 1994, which are hereby incorporated by reference in their entireties. Enhancer RNA molecules transcribed from these regions of DNA also “trap” transcription factors capable of binding DNA and RNA. A region with more than one enhancer is a “super-enhancer.”

INs provide a microenvironment for specific enhancer-gene interactions that are vital for both normal gene activation and repression. Transcriptional enhancers control over 20,000 protein-coding genes to maintain cell type-specific gene expression programs in all human cells. Tens of thousands of enhancers are estimated to be active in any given human cell type. See, ENCODE Project Consortium et al., Nature, 489, 57-74, 2012; Roadmap Epigenomics et al., Nature, 518, 317-330, 2015, which are hereby incorporated by reference in their entireties. Enhancers and their associated factors can regulate expression of genes located upstream or downstream by looping to the promoters of these genes. Cohesin ChIA-PET studies carried out to gain insight into the relationship between transcriptional control of cell identity and control of chromosome structure reveal that majority of the super-enhancers and their associated genes occur within large loops that are connected through interacting CTCF-sites co-occupied by cohesin. Such super-enhancer domains (SD) usually contain one super-enhancer that loops to one gene within the SD and the SDs appear to restrict super-enhancer activity to genes within the SD. The correct association of super-enhancers and their target genes in insulated neighborhoods is highly vital because the mis-targeting of a single super-enhancer is sufficient to cause disease. See Groschel et al., Cell, 157(2):369-81, 2014.

Most of the disease-associated non-coding variation occurs in the vicinity of enhancers and hence might impact these enhancers target genes. Therefore, deciphering the features conferring specificity to enhancers is important for modulatory gene expression. See, Ernst et al., Nature, 473, 43-49, 2011; Farh et al., Nature, 518, 337-343, 2015; Hnisz et al., Cell, 155, 934-947, 2013; Maurano et al., Science, 337, 1190-1195, 2012, which are hereby incorporated by reference in their entireties. Studies suggest that some of the specificity of enhancer-gene interactions may be due to the interaction of DNA binding transcription factors at enhancers with specific partner transcription factors at promoters. See, Butler and Kadonaga, Genes & Development, 15, 2515-2519, 2001; Choi and Engel, Cell, 55, 17-26, 1988; Ohtsuki et al., Genes & Development, 12, 547-556, 1998, which are hereby incorporated by reference in their entireties. DNA sequences in enhancers and in promoter-proximal regions bind to a variety of transcription factors expressed in a single cell. Diverse factors bound at these two sites interact with large cofactor complexes and interact with one another to produce enhancer-gene specificity. See, Zabidi et al., Nature, 518:556-559, 2015, which is hereby incorporated by reference in its entirety.

In some embodiments, enhancer regions may be targeted to after or elucidate GSNs. In certain embodiments, a GSC is modulated by targeting enhancer RNA molecules (eRNAs) from enhancer regions. For example, an occupancy-dependent signaling center (ODSC) comprising a nucleic acid sequence of SEQ ID NOs: 32,627-71,281 is targeted to modulate transcription of enhancer RNA molecules (eRNAs) from enhancer regions.

### Insulators

Insulators are regulatory elements that block the ability of an enhancer to activate a gene when located between them and contribute to specific enhancer-gene interactions. See, Chung et al., Cell 74:505-514, 1993; Geyer and Corces, Genes & Development 6:1865-1873, 1992; Kellum and Schedl, Cell 64:941-950, 1991; Udvardy et al., Journal of molecular biology 185:341-358, 1985, which are hereby incorporated by reference in their entireties. Insulators are bound by the transcription factor CTCF but not all CTCF sites function as insulators. See, Bell et al., Cell 98: 387-396, 1999; Liu et al., Nature biotechnology 33:198-203, 2015, which are hereby incorporated by reference in their entireties. The features that distinguish the subset of CTCF sites that function as insulators have not been previously understood.

Genome-wide maps of the proteins that bind enhancers, promoters and insulators, together with knowledge of the physical contacts that occur between these elements provide further insight into understanding of the mechanisms that generate specific enhancer-gene interactions. See, Chepelev et al., Cell research, 22:490-503, 2012; DeMare et al., Genome Research, 23:1224-1234, 2013; Dowen et al., Cell, 159:374-387, 2014; Fullwood et al., Genes & Development 6:1865-1873, 2009; Handoko et al., Nature genetics 43:630-638, 2011; Phillips-Cremins et al., Cell, 153:1281-1295, 2013; Tang et al., Cell 163:1611-1627, 2015, which are hereby incorporated by reference in their entireties. Enhancer-bound proteins are constrained such that they tend to interact only with genes within these CTCF-CTCF loops. The subset of CTCF sites that form these loop anchors thus function to insulate enhancers and genes within the loop from enhancers and genes outside the loop, as shown in FIG. 3B. In some embodiments, insulator regions may be targeted to after or elucidate GSNs.

Controlling Expression from Insulated Neighborhoods: Genomic Signaling Centers (GSCs)

GSCs have been discovered to regulate the activity of INs. These regions control which genes are expressed and the level of expression in the human genome by a context-specific combination of factors, such as signaling proteins, chromatin modifications, and transcriptional coactivators. GSCs permit drugs to control response by targeting signaling pathways. GSCs include enhancer regions bound by highly context-specific combinatorial assemblies that interact to form a three-dimensional transcription factor hub macrocomplex. GSCs mediate interactions of the regulatory elements of one to four genes in a gene loop organized by biological function. Loss of the structural integrity of GSCs contributes to deregulation of gene expression, which potentially causes disease.

GSCs may have a unique composition including chromatin modifications and binding by the assemblies of transcription factors, the transcription apparatus, and chromatin regulators. GSCs may be highly context specific. Multiple GSCs may interact to control different combinations of genes within the same IN.

In some embodiments of the invention herein, an ODSC is defined as a region of the genome bound by at least (i) 2 signaling proteins, and comprising (ii) a H3K27 chemical modification or independently at least one of a bromodomain-containing protein (Brd), a transcriptional coactivator, or at least two master transcription factors that control gene expression in an insulated neighborhood. In certain embodiments, the chemical modification is H3K27ac, a histone modification associated with active enhancers, and/or the bromodomain-containing protein is one of Brd2, Brd3, and Brd4. The definition of an ODSC may also be expressed as (H3K27ac or Brd4 or p300 or at least 2 master transcription factors)+at least 2 signaling proteins. Boundaries of the 38,659 occupancy-dependent signaling centers identified using this definition are shown in Tables 43-67. The nucleic acid sequences of the ODSCs are shown in SEQ ID NOs: 32,627-71,281.

### Binding Sites for Signaling Molecules

A series of consensus binding sites, or binding motifs for binding sites, for signaling molecules has been identified by the present inventors. These consensus sequences reflect binding sites along a chromosome, gene, or polynucleotide for signaling molecules or for complexes which include one or more signaling molecules. These sites are given in Table 11.

TABLE 11
Motifs for Binding Sites or Genomic Signaling Centers

| | SEQ | Gene | | SEQ | Gene | | SEQ | Gene |
|---|---|---|---|---|---|---|---|---|
| Motif Sequence | ID NO | symbol | Motif Sequence | ID NO | symbol | Motif Sequence | ID NO | symbol |
| AAAAGTCAA | 1 | NR2E1 | GGCVGTTR | 230 | MYB | NYMATTAN | 429 | MKX6-2 |
| AAAATGGCGCCATTTT | 2 | E2F2 | GNCACGTGYN | 231 | HEY2 | NYMATTAN | 429 | PDX1 |
| AAACCACAAA | 3 | RUNX1 | GTAATWAAAN | 232 | HOXC10 | NYNATTAN | 430 | BARX1 |
| AAAGTAAACA | 4 | FOXA1 | GTCACGCNNMATTAN | 232 | PAX3 | NYNATTAN | 430 | BSX |
| AAATGGCGCCATTT | 5 | E2F1 | GTCGTWAAAN | 234 | HOXC10 | NYTAATCCN | 431 | PITX1 |
| AACAGCTGTT | 6 | MSC | GTCGTWAAAN | 234 | HOXD11 | NYTAATCCN | 431 | PITX3 |
| AACATCTGGA | 7 | ZBTB18 | GTTAATNATTAAY | 235 | HNF1B | NYTAATTARN | 432 | LHX6 |
| AACCGGAAGT | 8 | ELF1 | TAATCRATAN | 236 | CUX1 | NYTAGTTAMN | 432 | HMBOX1 |
| AACCGGAAGT | 8 | ETV1 | TAATCRATAN | 236 | CUZ2 | NYYTGTTTACHN | 434 | FOXP1 |
| AACCGGAAGT | 8 | GABPA | TRTTTACTTW | 237 | FOXM1 | RACATATGTY | 435 | NEUROG2 |
| AATGGCGCCAAA | 9 | E2F1 | TSAAGGTCAN | 238 | ESRRG | RAGGTCAAAAGGTCAAKN | 436 | RARA |
| AATGGCGCCAAA | 9 | E2F4 | TTACGYAM | 239 | GMEB2 | RATAAAAR | 437 | CPEB1 |
| AATTGGCCAATTA | 10 | LBX2 | TTGGCANNNTGCCAR | 240 | NFIA | RCGGACACAATR | 438 | FOXG1 |
| ACAGGAAGTG | 11 | ERG | TTGGCANNNTGCCAR | 240 | NFIB | RGGATTAR | 439 | GSC |
| ACAGGAAGTG | 11 | ETS1 | A.yTrAAt | 241 | ARRB1 | RGGcAm | 440 | EGLN2 |
| ACATCCTGGT | 12 | SPDEF | AACAATNNNAKTGTT | 242 | SOX21 | RGGGCACTAACY | 441 | ZNF264 |
| ACCCTTGAACCC | 13 | ZNF524 | AACAATNNNNAKTGTT | 243 | SOX21 | RRGGTCAAAGTCCRNN | 442 | HNF4A |
| ACCGAACAAT | 14 | SOX17 | AACAATNNNNAKTGTT | 243 | SOX7 | RRGGTCAN | 443 | NR2F1 |
| aCGCGTc | 15 | MBL2 | AAGGKGRCGCAGGCA | 244 | ZNF165 | RRGGTCATGACCYY | 444 | RXRA |
| ACTACAATTCCC | 16 | GFY | ACCCTmAAGGTyrT | 245 | ZNF569 | RRGGTCATGACCYY | 444 | RXRG |
| ACTGAAACCA | 17 | IRF4 | ACNGTTAAACNG | 246 | MYBL1 | RTCACGTGAY | 445 | SREBF2 |
| ACTTTCACTTTC | 18 | PRDM1 | AkGmCACGTA | 247 | TRIM3 | RTCACGTGAY | 445 | TFEB |
| AGAAATGACTTCCCT | 19 | ZNF528 | AMCATATGKT | 246 | OLIG2 | RTCACGTGAY | 445 | TFEC |
| AGAGGAAGTG | 20 | MAZ | AMCATATGKT | 248 | OLIG3 | RTCACGTGAY | 445 | USF1 |
| aGATAAG | 21 | SLC6A1 | ANCATATGNT | 249 | BHLHE23 | RTGCCMNNN | 446 | HIC2 |
| AGGCCTAG | 22 | ZNF711 | ANCATATGNT | 249 | OLIG1 | SCACGTGS | 447 | MYCLP1 |
| AGGCCTGG | 23 | ZFX | ANRTAAAYAAACA | 250 | FOXC1 | SYMATTAN | 448 | HOXA6 |
| AGGTCTCTAACC | 24 | PRDM14 | ARRGGTCANNNRRGGTCA | 251 | RARA | VCAGCTGBNN | 449 | TCF12 |
| AGGTGTGA | 25 | MGAM | AWCAGCTGWT | 252 | TFAP4 | WATGCGCATW | 450 | POU5F1 |
| AGGTGTGA | 25 | TBX1 | AWNTAGGTCATGACCTANWT | 253 | RORB | WRCGTAACCACGYN | 451 | GMEB2 |
| AGGTGTGA | 25 | TBX15 | CCCGCNTNNNRCGAA | 254 | CENPB | YGCGCATGCGCN | 452 | NFE2L1 |
| AGGTGTGA | 25 | TBX4 | cCr.RTcrGG | 255 | ASCL1 | yGCGGCk. | 453 | SULT1A1 |
| AGGTGTGA | 25 | TBX5 | CGTRNAAARTGA | 256 | MSC | YRATTGCAATYR | 454 | LHX6 |
| AGGTGTGAAAAAAGGTGTGA | 26 | TBX1 | CNNBRGCGCCCCCTGSTGGC | 257 | CTCFL | YTAATTAN | 455 | VAX1 |
| AGGTGTGAAGGTGTGA | 27 | TBX20 | GACCACMCACNNNG | 258 | GLI2 | YTAATTAN | 455 | VAX2 |
| ATaacATG | 25 | SLC6A11 | GCCMCGCCCMC | 259 | AZF1 | YTAATTAN | 455 | VSX1 |
| ATAGTGCCACCTGGTGGCCA | 29 | CTCF | GCCMCGCCCMC | 259 | KLF16 | YTAATTAN | 455 | VSX2 |
| ATCAATAACATTGAT | 30 | SOX15 | GCCMCGCCCMC | 259 | PSG1 | ...a...gTAAACAa | 456 | FOXG1 |
| ATCAATTGCAGTGAT | 31 | SOX10 | GNCACGTGNC | 260 | HES1 | .gCsGgg | 457 | SLC13A4 |
| ATCACGTGAT | 32 | MLX | GRGGTCAAAAGGTCANA | 261 | RARG | DATGASTCAT | 458 | BATF |
| ATCACGTGAT | 32 | MLXIPL | GRGGTCAAAAGKTCAC | 262 | RARG | kGmCAGCGTGTC | 459 | TRIM3 |
| ATGAATAACATTCAT | 33 | SOX15 | GRGTTCANNRRGTTCA | 263 | CYP27B1 | NAACAATTKCAGTGTT | 460 | SOX4 |
| ATGAATTGCAGTCAT | 34 | SOX10 | GTCWGCTGTYYCTCT | 264 | ZNF317 | NATTTCCNGGAAAT | 461 | STAT1 |
| ATGACGTCAT | 35 | JUN | GTtgyCATgG.aAc | 265 | RFX1 | NCCGCCATNNT | 462 | YY2 |
| ATGACTCAT | 36 | BACH1 | GYCATCMATCAT | 266 | HOXA2 | NCYAATAAAA | 463 | HOXD13 |
| ATGACTCATC | 37 | FOS | TAATTAGYRYTRATTA | 267 | LHX6 | NGYAATAAAA | 464 | HOXD11 |
| ATGCCCTGAGGC | 38 | TFAP2A | TAATTRCYAATTA | 268 | LHX9 | NNCATCCCATAATANTC | 465 | ZNF410 |
| ATTGCGCAAC | 39 | CEBPA | TGAATNNNAKTCA | 269 | SOX21 | NNNGCATGTCCNGACATGCC | 465 | UVRAG |
| CAAGATGGCGGC | 40 | YY1 | TGAATRTKCAGTCA | 270 | SOX10 | NNNGMCACGTCATC | 467 | XBP1 |
| CACCGAACAAT | 41 | SOX6 | TGGKCTARCCTCGA | 271 | ZKSCAN3 | NRAAGGTCAANNNNRGGTCA | 468 | RORA |
| CCAAT | 42 | NFYA | TTCya.....TTC | 272 | HSF1 | NTGCCACGTCANCA | 469 | CREB3L1 |
| CCAAT | 42 | NFYB | tttCC.rAt.gg | 273 | SRF | NYAATTAAAANNYAATTA | 470 | MSX1 |
| CCAAT | 42 | NFYC | TTTCGCNTGGCNNGTCA | 274 | ZBTB49 | NYAATTAAAANNYAATTA | 470 | MSX2 |
| CCATGCCGCCAT | 43 | YY2 | TWCCCAYAATGCATTG | 275 | ZNF143 | RACATGTYNNRACATGTC | 471 | TP63 |
| CCCCGCGC | 44 | SLC13A4 | AGATSTNDNNDSAGATAASN | 275 | GATA3 | RAGGTCANTCAAGGTCA | 472 | ESRRA |
| CGCAGCTGCG | 45 | NHLH1 | ARGAGGMCAAAATGW | 277 | ZNF675 | RAGGTCANTCAAGGTCA | 472 | ESRRG |
| CGCGAAAa | 46 | VANGL2 | ATTTCCCAGVAKSCY | 278 | ZNF143 | RAGTCAANAAGTCA | 472 | NR2E1 |
| CGGACACAAT | 47 | FOXK1 | GGmraTA.CGs | 279 | APEX1 | rCGGC...rCGGC | 474 | SULT1A1 |
| CGGTTGCCATGGCAAC | 48 | RFX1 | AACAATRTKCAGWGTT | 280 | SOX10 | RGTTCRNNNRGTTC | 475 | NR1I2 |
| CGGTTTCAAA | 49 | CHR | AACAATRTKCAGWGTT | 280 | SOX8 | RMATWCCA | 476 | TEAD3 |
| CGTGGGTGGTCC | 50 | GLI3 | AACAATRTKCAGWGTT | 280 | SOX9 | rracGCsAaa | 477 | VANGL2 |
| CTAATTAG | 51 | PAX4 | ADGGYAGYAGCATCT | 281 | PRDM9 | RRGGTCANNNRRGGTCA | 476 | RARG |
| CTGCGCATGCGC | 52 | NFE2L1 | AGGTGTKANGGTGTSA | 282 | TBX20 | RTAAACAATAAAYA | 479 | FOXJ2 |
| CTTCGAG | 53 | XBP1 | AGGTGTKANNTMACACCT | 283 | MGAM | RTAAACARTAAACA | 480 | FOXJ3 |
| GAAAGTGAAAGT | 54 | IRF1 | AGGTGTNANWWNTNACACCT | 284 | TBX4 | RTAAACAWMAACA | 481 | FOXJ2 |
| GAGGTCAAAGGTCA | 59 | NR2C2 | AGGTGTNANWWNTNACACCT | 284 | FBX5 | RTAAAYA | 482 | FOXD2 |
| GATGACGTCATC | 56 | XBP1 | ATCAATRTKCAGWGAT | 285 | SOX9 | RTAAAYA | 482 | FOXD3 |
| GATGACTCAGCA | 57 | NFE2 | ATCRATNNNNNATCRAT | 286 | CUX1 | RTAAAYA | 482 | FOXL1 |
| GATTGCATCA | 58 | APEH | ATCRATNNNNNNATCRAT | 287 | CUZ1 | RTAAAYAAACA | 482 | FOXC2 |
| GCACTTAA | 59 | ISL2 | ATCRATNNNNNNATCRAT | 287 | CUZ2 | rTGTAcGGrT | 484 | FHL1 |
| GCAGCCAAGCGTGACC | 60 | PAX5 | ATGAATRTKCAGWCAT | 286 | SOX9 | RTGTTAAAYGTAGATTAAG | 485 | ZNF232 |
| GCTAATCC | 61 | CRX | CCTCATGGTGYCYTWYTCCCTT | 289 | ZNF41 | WRTAAAYAAACAA | 486 | FOXL1 |
| | | | GTG | | | | | |
| GCTTTTCCCACA | 62 | ZNF75A | GACCMCCYRMTGNG | 290 | ZIC1 | WTTCGAAYG | 487 | HSFY2 |
| ggACCCt | 63 | NRG1 | gcsGsg..sG | 291 | SLC13A4 | YACGTAACNKACGTA | 486 | GMEB2 |
| GGCCATAAATCA | 64 | HOXA9 | GGMTNAKCC | 292 | RHOXF1 | yGGCGCTAyca | 489 | SNTB1 |
| GGGGGTGTGTCC | 65 | KLF10 | GGTGTNANWWNTNACACC | 293 | TBX2 | YTGGCANNNTGCCAA | 490 | NFIX |
| GGGGTCAAAGGTCA | 66 | RXRA | GGYGTGANNNNTCACRCC | 294 | MGAM | YTGGCNNNNTGCCAA | 491 | NFIX |
| GGGGTCAAAGGTCA | 66 | RXRB | GRTGMTRGAGCC | 295 | ZNF415 | ...ACCCR.aCMy | 492 | RAP1A |
| GGGGTCAAAGGTCA | 66 | RXRG | TAAWYGNNNNTAAWYG | 296 | BARHL2 | .tGrTAGCGCCr... | 492 | SNTB1 |
| GGTCACGTGA | 67 | USF1 | TGAATRTKCAGWCA | 297 | SOX8 | KCTAWAAATAGM | 494 | MEF2A |
| GGTCCCTAGGGA | 68 | EBF1 | TGMATNNNTNA | 298 | POU3F4 | MCCATATAWGGN | 495 | SRF |
| GTAAACA | 69 | FOXI1 | tGyayGGrtg | 299 | RAP1A | NAAACGATNNAN | 496 | HOMEZ |
| GTAAACA | 69 | FOXO4 | tktCC..wTtGGAAA | 300 | SRF | NAACAATANCATTGTTN | 497 | SOX2 |
| GTAMCA | 69 | FOXO6 | TNACACCTNNNAGGTGTNA | 301 | TBX21 | NAACAATKNYAGTGTTN | 496 | SOX8 |
| GTAAACAA | 70 | FOXO1 | TRGGTYASTAGGTCA | 302 | NR1D2 | NAACAATKNYAGTGTTN | 496 | SOX9 |
| GTAAACATGTTTAC | 71 | FOXO1 | TYTCACACCTNNNAGGTGTGAR | 303 | TBX1 | NAACAATNNNATTGTTN | 499 | SOX2 |
| | | | A | | | | | |
| GTAAACATGTTTAC | 71 | FOXO3 | AAACYKGTTWDACMRGTTTB | 304 | GRHL2 | NAACCGCAAACCRCAN | 500 | RUNX2 |
| GTAAACATGTTTAC | 71 | FOXO4 | GACCCCCYGYTGNGN | 305 | ZIC3 | NAACCGCAAACCRCAN | 500 | RUNX3 |
| GTAAACATGTTTAC | 71 | FOXO6 | GACCCCCYGYTGNGN | 305 | ZIC4 | NAACCRCAAN | 501 | RUNX3 |
| GTAATAAAA | 72 | HOXD12 | TTTYACGCWTGANTGMNYN | 306 | PAX6 | NAAGACGYCTTN | 502 | PROX1 |
| GTACCTACCT | 73 | ZNF784 | ACCYTmArGGT.rTG | 307 | ZNF569 | NAANCGAAASYR | 503 | IRF2 |
| GTCACGTG | 74 | RBPJ | CNATTAAAWANCNATTA | 308 | BARX1 | NAASATCAAAGN | 504 | TCF7L1 |
| GTCACGTGAC | 75 | ARNTL | TAGAYRAYTGMCANGAA | 309 | ZNF713 | NACAYATGNN | 505 | TCF15 |
| GTCACGTGAC | 75 | BHLHE41 | TWGHWACAWTGTWDC | 310 | DMRT1 | NACTTCCGSCGGAARMN | 506 | ELK1 |
| GTCCCCAGGGGA | 76 | EBF1 | GNCTGTASTRNTGBCTCHTT | 311 | ZNF382 | NAGGTCAMSRTGACCTN | 507 | ESR1 |
| GTGCGCATGCGC | 77 | NKRF | ....gTAAACAa | 312 | FOXO1 | NAMCCGGAAGTR | 506 | ELF2 |
| GTGGGCCCCA | 76 | ZNF692 | KCCACGTGAC | 313 | NPAS2 | NATCAATANCATTGATN | 509 | SOX2 |
| TCACACCTAGGTGTGA | 75 | Tr | MCGCCCACGCA | 314 | EGR2 | NATCAATKNYAGTGATN | 516 | SOX8 |
| tCACGTG | 80 | RBPJ | NACCCGGAAGTA | 315 | EHF | NATCAATKNYAGTGATN | 510 | SOX9 |
| TGACCTTTAAAGGTCA | 81 | NR4A2 | NAGGTGTGAA | 316 | TBX21 | NATCAATNNNATTGATN | 511 | SOX2 |
| TGATGACGTCATCA | 82 | BATF3 | NATGCGGGTA | 317 | GCM1 | NATGAATKNYAGTCATN | 512 | SOX9 |
| TGCCACGTGGCA | 82 | CREB3L1 | NCCACGTG | 318 | MYC | NATGASTCATN | 512 | NFE2 |
| tGCTGGT | 84 | ACE2 | NCCACTTAA | 319 | NKX3-1 | NCACTTNAN | 514 | NKX2-8 |
| TGGCAGTTGG | 85 | MYBL1 | NCCACTTAA | 319 | NKX3-2 | NCCACTTRAN | 515 | NKX2-3 |
| TTACGTAA | 86 | GMEB2 | NCCCCCCCAC | 320 | ZNF740 | NCCGGAWATCCGGN | 516 | ERG |
| TTACTAA | 87 | ARRB1 | NCGAAACCGAAACT | 321 | RF8 | NCCGGAWRYRYWTCCGGN | 517 | ETS1 |
| TTgccATggCAAC | 88 | RFX1 | NNCACGTGCC | 322 | HEY2 | NCCGGNNNNNNCCGGN | 518 | TFCP2 |
| tTgTTTac | 89 | FOXO1 | NNGCGGAAGTG | 323 | ETV7 | NCGAAANYGAAACY | 519 | IRF7 |
| TTTAAAGGTCA | 90 | NR4A2 | NTGGGTGTGGCC | 324 | KLF1 | NCGACCACCNAN | 520 | ZBTB7C |
| TTTCCCCACAC | 91 | FOXO3 | NTTTCCAGGAAA | 325 | STAT4 | NCTAWAAATAGM | 521 | MEF2D |
| TTTCCCCACACG | 92 | FOXO1 | rACGCGt | 326 | MBL2 | NGACCCCCCACGANGN | 522 | GLIS1 |
| TTTCCCCACACG | 92 | FOX04 | rCACAAT | 327 | MAT1A | NGAGGTCANNGAGTTCANNN | 523 | CYP27B1 |
| TTTCCCGCCAAA | 92 | E2F8 | RCCGGAAGTG | 328 | ETS2 | NGCCNNNNGGCN | 524 | TFAP2A |
| TTTGGCGCCAAA | 94 | E2F1 | RRGGTCAAAAGGTCA | 329 | NR2F6 | NGCCNNNNGGCN | 524 | TFAP2B |
| TTTGGCGCCAAA | 94 | E2F4 | RRGGTCAAAGGTCA | 330 | HNF4A | NGCCNNNNGGCN | 524 | TFAP2C |
| TTTTCCATGGAAAA | 95 | NFATC1 | RRGGTCAAAGGTCA | 330 | NR2C2 | NGCCNNNNNGGCN | 525 | TFAP2C |
| AAAAAGCGGAAGTN | 96 | SPI1 | RRGGTCAAAGGTCA | 330 | NR2F6 | RGGGGAWTCCCCN | 526 | NFKB1 |
| AAAAATGGCGCCAAAANN | 97 | E2F2 | RRGTCCAAAGGTCAA | 331 | HNF4A | NGGGGAWTCCCCN | 526 | NFKB2 |
| AAAGATCAAAGGRWW | 98 | LEF1 | RTAAACA | 332 | FOXP3 | NGTCGTWAAAN | 527 | HOXC11 |
| AACCCGGAAGTN | 99 | ELF1 | RTAAACAA | 333 | FOXJ2 | RGTCGTWAAANN | 528 | HOXA10 |
| AACCCGGAAGTR | 100 | ELF1 | RTAAACAA | 333 | FOXJ3 | NHAACBGYYV | 529 | MYBL2 |
| AACCCGGAAGTR | 100 | ELF4 | RTAAACATAAACA | 334 | FOXJ3 | NMCGCCCMCGCANN | 530 | 2GR1 |
| AACCGTTAACGGNN | 101 | MYBL2 | RTCATGTGAC | 335 | MITF | NMCRATTAR | 531 | VENTX |
| ACCCGGAAGTN | 102 | ELF5 | VDTTTCCCGCCA | 336 | E2F7 | NNAAAATCRATANN | 532 | ONECUT3 |
| ACCGGAAGTN | 103 | ELK1 | WACCCGGAAGTA | 337 | ELF3 | NNAAAATCRATAWN | 533 | ONECUT1 |
| ACCGGAAGTN | 103 | ELK3 | WDNCTGGGCA | 338 | ZNF416 | NNAAAATCRATAWN | 533 | ONECUT2 |
| ACCGGAAGTN | 103 | ERF | wwwwsyGGGG | 339 | VPS4B | NNATGAYGCAATN | 534 | ATF4 |
| ACCGGAAGTN | 103 | ETV3 | YGTCTAGACA | 340 | SMAD3 | NNCAMTTAANN | 535 | HMX1 |
| ACCGGAAGTN | 103 | ETV4 | yTGACT | 341 | ASCL1 | NNCAMTTAANN | 535 | HMX2 |
| ACCGGAAGTN | 103 | FEV | .gcTgAcTAA. | 342 | YAP1 | NNCAMTTAANN | 535 | HMX3 |
| ACCGGAAGTN | 103 | GABPA | DNNNGGTCANNNH | 342 | NR2F1 | NNMCCATATAWGGKNN | 536 | SRF |
| ACCGGAARYN | 104 | ETS1 | HAATAAAGNN | 344 | PABPC1 | NNNNTGCTGASTCAGCANNNN | 537 | MAFG |
| ACCGGAARYN | 104 | ETV1 | HNHNAGGTGTGANHH | 346 | TBX20 | NNNNTGCTGASTCAGCANNNN | 537 | MAFK |
| ACCGGAARYN | 104 | FLI1 | kGCTGr | 346 | SWI5 | NNRTGACGTCAYCN | 538 | CREB3 |
| AGATAAN | 105 | GATA1 | KSYGGAAGTN | 347 | ETV6 | NNTAATCCGMTTANN | 539 | OTX2 |
| AGATAANN | 106 | GATA3 | NAAACCGGTTTN | 348 | GRHL1 | NNTCCCNNGGGANN | 540 | EBF1 |
| AGATAANN | 106 | GATA4 | NAACAATRN | 349 | SOX9 | NNTNATTANN | 541 | EVX1 |
| AGATAANN | 106 | GATA5 | NAACCGCAAN | 350 | RUNX3 | NNTNATTANN | 541 | EVX2 |
| AGATAASR | 107 | GATA3 | NAACCGGTTN | 351 | GRHL1 | NNYATGMATAATTAATN | 542 | POU1F1 |
| AGGTGTGANN | 108 | TBR1 | NAACCGGTTN | 351 | TFCP2 | NRGGTCANNGGTCAN | 543 | NR2F1 |
| AGGTGTNR | 109 | TBX3 | NACACGTGTN | 352 | CLOCK | NRMATWCCWN | 544 | TEAD1 |
| CCAATAAAAN | 110 | HOXA13 | NACCCGGAAGTAN | 353 | ELF3 | NRNWAAYRTTKNYN | 545 | FOXD2 |
| CCAATAAAAN | 110 | HOXB13 | NACCGAAACYN | 354 | IRF5 | NRNWAAYRTTKNYN | 545 | FOXD3 |
| CCAATAAAAN | 110 | HOXC13 | NACGCCCACGCANN | 355 | EGR1 | NRTTAATNATTAACN | 546 | HNF1A |
| CCGAAACCGAAACY | 111 | IRF5 | NAGGTGTGAAAWN | 356 | EOMES | NRTTAATNATTAACN | 546 | HNF1B |
| cTCGAGG. | 112 | XBP1 | NAGGTGTGAAN | 357 | TBR1 | NRTTACRTAAYN | 547 | NFIL3 |
| gCTTCCw | 113 | NR3C1P1 | NAGGTGTGAAN | 357 | TBX20 | NRTTACRTAAYN | 547 | TEF |
| GGCGGGAAAH | 114 | E2F4 | NAGGTGTGAWN | 358 | TBX2 | NSCCNNNGGSN | 548 | TFAP2A |
| GGCGGGAARN | 115 | E2F6 | NATATGCTAATKN | 359 | POU5F1 | NSCCNNNGGSN | 548 | TFAP2B |
| GHCACGTG | 116 | CLOCK | NATGACGTCAYN | 360 | JDP2 | NSCCNNNGGSN | 548 | TFAP2C |
| GNCACGTG | 117 | ARNTL | NATGCGGAAGTR | 361 | ELF2 | NSCCNNNNNGGSN | 549 | TFAP2A |
| GTAAACAW | 118 | FOXO3 | NATGCGGGTAN | 362 | GCM1 | NSCCNNNNNGGSN | 549 | TFAP2B |
| TCAAGGTCAWN | 119 | ESRRB | NATGCGGGTN | 363 | GCM2 | NSCCNNNNNGGSN | 549 | TFAP2C |
| tgCACCy | 120 | FAM64A | NCCGCCATTNN | 364 | YY1 | NSCTMATTAN | 550 | POU6F2 |
| tgCATTT. | 121 | CST6 | NCCGTTAACGGN | 365 | MYBL1 | NTAATTANNNTAATTAN | 551 | MEOX2 |
| tGCTGg.. | 122 | SW15 | NCGAAACCGAAACY | 366 | IRF8 | NTAATYNRATTAN | 552 | ALX3 |
| TGCTTTCTAGGAATTMM | 123 | BCL6B | NCGAAACCGAAACYN | 367 | IRF4 | NTAATYNRATTAN | 552 | ALX4 |
| TGGGGAAGGGCM | 124 | ZNF467 | NCGACCACCGAN | 368 | ZBTB7B | NTAATYNRATTAN | 552 | UBA2 |
| TTGACAGS | 125 | MEIS2 | NCTAATTANN | 369 | LHX2 | NTAATYTAATTAN | 553 | ISX |
| TTTTCCCGCCAAAW | 126 | E2F7 | NCTCGTAAAAN | 370 | HOXA13 | NTAATYTAATTAN | 553 | UNCX |
| aaa.GTAAACAa | 127 | FOXG1 | NCTCGTAAAAN | 370 | HOXB13 | NTAAYYNAATTAN | 554 | DRGX |
| AACAATAMCATTGTT | 128 | SOX15 | NCTCGTAAAAN | 370 | HOXC13 | NTAAYYYAATTAN | 555 | ALX1 |
| AACAATANCATTGTT | 129 | SRY | NCTCGTAAAAN | 370 | HOXD13 | NTATCGCGAYATR | 556 | ZBED1 |
| AACAATKNYAGTGTT | 130 | SOX8 | NGATGACGTCATCR | 371 | FOS | NTATGCWAATN | 557 | POU2F2 |
| AACAATNNCATTGTT | 131 | SOX18 | NGCGACCACCNN | 372 | ZBTB7A | NTATYGATCH | 556 | ONECUT1 |
| AACAATNNNAGTGTT | 132 | SOX10 | NGGCACGTGCCN | 373 | HES5 | NTGAATNNCATTCAN | 559 | SOX14 |
| AACAATNNNCATTGTT | 133 | SRY | NGGCACGTGCCN | 373 | HES7 | NTGAATNNNATTCAN | 560 | SOX2 |
| AACAVBTGTT | 134 | MYF6 | NGTCGTAAAAN | 374 | HOXC12 | NTGAATWNCATTCAN | 561 | SOX2 |
| AACCGGWNNNWCCGGTT | 135 | GRHL1 | NGTCGTAAAAN | 374 | HOXD12 | NTGACCTNNNNNAGGTCAN | 562 | THRB |
| AAGAACATWHTGTTC | 136 | PGR | NKCACGTGMN | 375 | BHLHE40 | NTGCCACGTCAYCN | 563 | CREB3 |
| AAGGTCANNNNAAGGTCA | 137 | ESRRG | NKCACGTGMN | 375 | BHLHE41 | NTGCTGANTCAGCRN | 567 | MAFK |
| AANTAGGTCAGTAGGTCA | 138 | RORB | NNAAATGGCGCCAAAANN | 376 | E2F3 | NTGCTGASTCAGCAN | 565 | MAFF |
| AARGGTCAAAAGGTCA | 139 | RARB | NNAAATGGCGCCATTTNN | 377 | E2F3 | NTGMATAATTAATGAN | 566 | POU4F3 |
| ACAATANCATTG | 140 | SOX14 | NNAACCGTTNN | 378 | MYBL1 | NTMATCATCATTAN | 567 | MEOX2 |
| ACCGGAWATCCGGT | 141 | ERG | NNAACCGTTNN | 378 | MYBL2 | NTTRCGCAAY | 568 | CEBPB |
| ACCGGAWATCCGGT | 141 | FLI1 | NNACGCCCACGCANN | 379 | EGR3 | NTTRCGCAAY | 568 | CEBPD |
| ACCGTTARRACCGTTA | 142 | MYBL2 | NNACGCCCACGCANNN | 380 | EGR4 | NTTRCGCAAY | 568 | CEBPE |
| ACTACTAwwwwTAG | 143 | TMEM50A | NNATGACGTCATNN | 381 | ATF7 | NTTRCGCAAY | 566 | CEBPG |
| ACVAGGAAGT | 144 | ELF5 | NNCACCTGCN | 382 | TCF3 | NWACAYRACAWN | 569 | IRX2 |
| AGATGKDGAGATAAG | 145 | GATA3 | NNCACCTGNN | 383 | FIGLA | NWACAYRACAWN | 569 | IRX5 |
| AGGTCANNNNNTGACCT | 146 | NR4A2 | NNCACCTGNN | 383 | MESP1 | NWTATGCWAATN | 570 | POU2F1 |
| AGGTGTGAANTTCACACCT | 147 | TBX15 | NNCACCTGNN | 383 | TCF3 | NWTATGCWAATTW | 571 | POU3F3 |
| AGGTGTGAAWTTCACACCT | 148 | TBX1 | NNCACCTGNN | 383 | TCF4 | NYAATTARNNNYAATTAN | 572 | PDX1 |
| AGGTGTGAWWWTCACACCT | 149 | TBX20 | NNCACGTGNN | 384 | HEY1 | MYTMATTANN | 573 | NOTO |
| AGTGTTAACAGARCACCT | 150 | ZSCAN16 | NNCACGTGNN | 384 | MAX | RACCGTTAAACNGYY | 574 | MYBL2 |
| ANACAGCTGC | 151 | TCF3 | NNCACGTGNN | 384 | MNT | RCATTCCNNRCATTCCN | 575 | TEAD3 |
| ANCAGCTG | 152 | KRT7 | NNCACGTGNN | 384 | TFE3 | RCTAWAAATAGM | 576 | BORCS8- |
| | | | | | | | | MEF2B |
| ARGGTCAAAAGGTCA | 153 | RARA | NNCATATGNN | 385 | BHLHA15 | RRGGTCANNNGGTCAN | 577 | NR2F1 |
| ATAAACAANWGTAAACA | 154 | FOXG1 | NNCATATGNN | 385 | NEUROD2 | RTMATTAN | 578 | HOXA4 |
| ATCAATNNCATTGAT | 155 | SOX18 | NNCCGCCATNW | 386 | YY2 | SCTMATTANN | 579 | POU6F2 |
| ATGAATKKTAGTC | 156 | SOX8 | NNGCAACAGGTGGNN | 387 | SCRT1 | WAACCRCAN | 580 | RUNX2 |
| ATGAATWYCATTCAT | 157 | SOX18 | NNGCAACAGGTGN | 388 | SCRT2 | WGTAAAYAN | 581 | FOXB1 |
| ATGASTCAT | 158 | JDP2 | NNGGAAGTGCTTCCNN | 389 | ETV7 | WTATGCWAATNW | 582 | POU3F1 |
| ATGCGGGYRCCCGCAT | 159 | GCM1 | NNGGATTANN | 390 | DMBX1 | YAATTANNCTAATTR | 583 | LHX2 |
| ATGMATAATTAATG | 160 | POU4F1 | NNGGATTANN | 390 | DPRX | YMATTARYTAATKR | 584 | EMX1 |
| ATTTCTNAGAAA | 161 | STAT5A | NMMCGCCCACGCANN | 391 | EGR2 | YMATTARYTAATKR | 584 | EMX2 |
| AVCAGGAAGT | 162 | EHF | NNMCGCCCACGCANNN | 392 | EGR4 | YTCACACCTNNAGGTGTGAR | 585 | TBX1 |
| CACGTGNNNNNCACGTG | 163 | MAX | NNNNNTGCTGAN | 393 | MAFK | DGGGYGKGGC | 586 | KLF5 |
| CGatAa..sC | 164 | APEX1 | NNNNTGCTGAN | 394 | NRL | NGACCACMCACGWNG | 587 | GL13 |
| CGG...........cCg | 165 | LGALS4 | NNNTGGCGCCANNN | 395 | E2F1 | NGCCACGCCCMCNT | 588 | SP6 |
| CGGas.rsw.y.s.CCGa | 166 | LGALS4 | NNRRAAAGGAAACCGAAACTN | 396 | IRF3 | NTGMATAATTAATKAG | 589 | POU4F2 |
| CTTGGCACNGTGCCAA | 167 | NF1 | NNTAAACGNN | 397 | BARHL2 | NWTATGCWAATKAG | 590 | POU1F1 |
| CWGGCGGGAA | 168 | E2F1 | NNTAATCCGATTANN | 398 | OTX1 | rGAA..TtctrGAA | 591 | HSF1 |
| GACCCCCCGCGNNG | 169 | GLIS2 | NNTAATCCNN | 399 | GSC | RGGTCANNNARRGGTCA | 592 | RARA |
| GACCCCCCGCGNNG | 169 | GLIS3 | NNTAATCCNN | 399 | GSC2 | RGKGGGCGKGGC | 593 | SP6 |
| GAGSCCGAGC | 170 | ZVF519 | NNTAATTANN | 400 | HOXD8 | RRGGTCANNNARAGGTCA | 594 | RARG |
| GARTGGTCATCGCCC | 171 | ZNF669 | NNTAATTGNN | 401 | BARHL2 | rTCAyt....Acg | 595 | MSC |
| GCGACCACNCTG | 172 | GLI2 | NNTAATTRNN | 402 | HESX1 | rTGTayGGrtg | 596 | FHL1 |
| ggGTGca.t | 173 | FAM64A | NNTCCAGATGTKN | 403 | ZBTB18 | SAAGGTCANNTSAAGGTCA | 597 | ESRRA |
| GGGTTTTGAAGGATGARTAGG | 174 | ZFP3 | NNTGCCAAN | 404 | NFIX | sc.GC.gg | 598 | EGLN2 |
| AGTT | | | | | | | | |
| GGNTCTCGCGAGAAC | 175 | ZBTB33 | NNTGCCAANN | 405 | NFIA | SMCAGTCWGAKGGAGGAGGC | 599 | ZSCAN22 |
| GGTGTGANNTCACACC | 176 | TBX21 | NNTTTTGGCGCCAAAANN | 406 | E2F2 | WTATGCWAATKA | 600 | POU3F2 |
| GGTGTGAWATCACACC | 177 | TBX21 | NNTTTTGGCGCCAAAANN | 406 | E2F3 | .rTCAyty..ACG. | 601 | MSC |
| GNCCACGTGG | 178 | MYC | NNYAATTANN | 407 | ALX3 | MACCTTCYATGGCTCCCTAKTG | 602 | ZNF16 |
| | | | | | | CCY | | |
| GSCTGTCACTCA | 179 | PBX1 | NNYAATTANN | 407 | DLX1 | NAACAATKNYAKTGTTN | 603 | SOX7 |
| GTCACGCTCNCTGA | 180 | PAX5 | NNYAATTANN | 407 | EMX1 | NANCATATGNTN | 604 | BHLHE22 |
| GTGGTCCCGGATYAT | 181 | SPDPF | NNYAATTANN | 407 | EMX2 | NATGAATKNYAKTCATN | 605 | SOX7 |
| GYAATAAAA | 182 | HOXC12 | NNYAATTANN | 407 | EN1 | NATGGAAANWWWWTTTYCMN | 606 | NFATC1 |
| TAATTRNNYAATTA | 183 | GBX2 | NNYAATTANN | 407 | EN2 | NCAGNAAGAMGTAWMM | 607 | SPDEF |
| TAATTRSNYAATTA | 184 | EN1 | NNYAATTANN | 407 | ESX1 | NGCGCCMYCTAGYGGTN | 608 | CTCF |
| TAATYNAATTA | 185 | PROP1 | NNYAATTANN | 407 | GBX1 | NGNTCTAGAACCNGV | 609 | ZBTB12 |
| TAATYRATTA | 186 | PAX3 | NNYAATTANN | 407 | GBX2 | NGTCACGCWTSRNTGNNY | 610 | PAX2 |
| TAATYRATTA | 186 | PAX7 | NNYAATTANN | 407 | HOXA1 | NGTCACGCWTSRNTGNNY | 610 | PAX5 |
| TAATYTAATTA | 187 | PRRX1 | NNYAATTANN | 407 | LBX2 | NGTTNCCATGGNAACN | 611 | RFX3 |
| TAATYYAATTA | 188 | PHOX2A | NNYAATTANN | 407 | MIXL1 | NGTTNCCATGGNAACN | 611 | RFX4 |
| TAATYYAATTA | 188 | PHOX2B | NNYAATTANN | 407 | MNX1 | NGTTRCCATGGYAACN | 612 | RFX2 |
| TAAYYNAATTA | 189 | PROP1 | NNYAATTANN | 407 | PRKRA | NGTTRCCATGGYAACN | 612 | RFX5 |
| TATGCWAAT | 190 | POU2F3 | NNYMATTANN | 408 | GSX1 | NGYAATWAAAN | 613 | HOXA10 |
| TATGCWAAT | 190 | POU3F4 | NNYMATTANN | 408 | GSX2 | NGYAATWAAAN | 613 | HOXC11 |
| TATGCWAAT | 190 | POU5F1B | NNYMATTANN | 408 | HOXA2 | NMCMCGCCCMCN | 614 | SP8 |
| TCAATANCATTGA | 191 | SRY | NNYMATTANN | 408 | HOXB2 | NNGNNACGCCCMYTTTNN | 615 | KLF13 |
| TCAATNNCATTGA | 192 | SOX14 | NNYMATTANN | 408 | HOXB3 | NRTMAATATTKAYN | 616 | FOXC1 |
| TCAATNNNATTGA | 193 | SOX21 | NNYMATTANN | 408 | HOXB5 | NRTMAATATTKAYN | 616 | FOXC2 |
| TCACACCTNNNNAGGTGTGA | 194 | EOMES | NNYMATTANN | 408 | HOXD4 | NTAATTRGYAATTAN | 617 | HESX1 |
| TGAATANCATTCA | 195 | SRY | NNYTAATTANN | 409 | VSX1 | NTGACCTNATNAGGTCAN | 618 | THRA |
| TGACAGSTGTCA | 196 | MEIS3P1 | NNYTTCCGGGAARNR | 410 | ETV7 | NTGACCTNATNAGGTCAN | 618 | THRB |
| TGACAGSTGTCA | 196 | PKNOX1 | NRAAAGTGAAAGTGN | 411 | PRDM1 | NTGTTTACRGTAAAYAN | 619 | FOXI1 |
| TGACAGSTGTCA | 196 | PKNOX2 | NRCACCTGNN | 412 | ID4 | NTRAKCCN | 620 | RHOXF1 |
| TGACAGSTGTCA | 196 | TGIF1 | NRCAGGTGN | 413 | SNAI2 | NTRAYNTAATCAN | 621 | DUXA |
| TGACAGSTGTCA | 196 | TGIF2 | NRCATTCCWN | 414 | TEAD4 | NWRGCCMCGCCCMCTNN | 622 | SP4 |
| TGACAGSTGTCA | 196 | TGIF2LX | NRGATAATCYN | 415 | DPRX | NWTRMATATKYAWN | 623 | POU2F1 |
| TGCACACNCTGAAAA | 197 | ZSCAN4 | NRGTCCAAAGTCCANY | 416 | HNF4A | NWTRMATATKYAWN | 623 | POU2F2 |
| TGGAACAGMA | 198 | ZNF189 | NRTTACGTAAYN | 417 | DBP | RCATTCCNNRCATWCCN | 624 | TEAD1 |
| TRNGTAAACA | 199 | FOXA1 | NRTTACGTAAYN | 417 | EPAS1 | RTGGAAAANTMCNN | 625 | NFAT5 |
| TTaGTmAGc | 200 | YAP1 | NRYTTCCGGH | 418 | FLII | SGTTGCYARGCAACS | 626 | RFX2 |
| TTCCKNAGAA | 201 | STAT6 | NSTAATTANN | 419 | HOXA3 | SGTTGCYARGCAACS | 626 | RFX3 |
| TTCGAANNNTTCGAA | 202 | HSFY2 | NSTAATTANN | 419 | MEOX1 | SGTTGCYARGCAACS | 626 | RFX4 |
| TTCTAGAANNTTC | 203 | HSF1 | NSTAATTANN | 419 | MEOX2 | SGTTRCCATRGCAACS | 627 | RFX5 |
| TTCTAGAANNTTC | 203 | HSF2 | NTAATCCN | 420 | OTX1 | SSYTAATCGRWAANCGATTAR | 628 | VENTX |
| TTCTAGAANNTTC | 203 | HSF4 | NTAATCCN | 420 | OTX2 | w.TTRamkAR | 629 | SPTLC2 |
| TTGACAGSTGTCAA | 204 | MEIS2 | NTAATCCN | 420 | PITX1 | WAWRTAAAYAWW | 630 | FOXC2 |
| TTTCAAGGCYCCC | 205 | PRDM4 | NTAATTAN | 421 | LMX1A | WNWGTMAATATTRACWNW | 631 | FOXB1 |
| TTTCCAYNRTGGAAA | 206 | NFATC1 | NTCAAGGTCAN | 422 | ESRRA | WRWGTMAAYAN | 632 | FOXB1 |
| TTTCCCCACACRAC | 207 | FOXO6 | NTGACAGN | 423 | MEIS3P1 | WRWRTMAAYAW | 633 | FOXC1 |
| TTTTATKRGG | 206 | HOXB13 | NTGACAN | 424 | MEIS1 | WTAATKAGCTMATTAW | 634 | POU6F2 |
| AAAAAGMGGAAGTN | 209 | SPIB | NTTAATCCN | 425 | PITX1 | ymtGTmTytAw | 635 | SPTLC2 |
| AAAAAGNGGAAGTN | 210 | SPIC | NWAACCACADNN | 426 | RUNX2 | NGTGTTCAVTSAAGCGKAAA | 636 | PAX6 |
| AACCGGAARTR | 211 | ETV2 | NYAATTAN | 427 | DLX2 | NWTGMATAAWTNA | 637 | POU3F2 |
| ACCGGAARTN | 212 | ELK4 | NYAATTAN | 427 | DLX3 | SGTCACGCWTGANTGMA | 638 | PAX1 |
| ACCGGAARTN | 212 | ERG | NYAATTAN | 427 | DLX4 | SGTCACGCWTGANTGMA | 638 | PAX9 |
| ACCGGAWATCCGGN | 212 | FLI1 | NYAATTAN | 427 | DLX5 | VAGRACAKNCTGTBC | 639 | PGR |
| ACCGGAWGYN | 214 | ETV5 | NYAATTAN | 427 | DLX6 | WAWNTRGGTYAGTAGGTCA | 640 | RORA |
| AGGTCANTGACCTN | 215 | NR1H4 | NYAATTAN | 427 | EN1 | WTGMATAAWTNA | 641 | POU3F1 |
| AMCCGGATGTN | 216 | SPDEF | NYAATTAN | 427 | ISX | NTGMATAAWTNA | 641 | POU3F3 |
| AVYTATCGATAD | 217 | ZBED1 | NYAATTAN | 427 | LHX9 | WTRMATATKYAW | 642 | POU2F3 |
| AWCGAAACCGAAACY | 218 | IRF9 | NYAATTAN | 427 | LMX1B | WTRMATATKYAW | 642 | POU3F3 |
| CCAAT.a. | 219 | NFYA | NYAATTAN | 427 | MSX1 | NTRMATATKYAW | 642 | POU5F1B |
| CCCATWAAAN | 220 | HOXC10 | NYAATTAN | 427 | MSX2 | YTKGATAHAGTATTCTWGGTN | 643 | 2NF136 |
| | | | | | | GGCA | | |
| CCGGAASCGGAAGTN | 221 | ETV6 | NYAATTAN | 427 | PRRX1 | NNCNGTTNNNACNGTTN | 644 | MYBL1 |
| CCWGGAATGY | 222 | TEAD2 | NYAATTAN | 427 | PRRX2 | NRGNACANNNTGTNCYN | 645 | NR3C2 |
| CCWGGAATGY | 222 | TEAD4 | NYAATTAN | 427 | RAX2 | NRGWACANNNTGTWCYN | 646 | VR3C1 |
| CGSGTAA. | 223 | VANGL1 | NYAATTAN | 427 | SHOX | NTGACCTYANNTRAGGTCAN | 647 | THRB |
| CTCGRACCCGTGCN | 224 | ZNF524 | NYAATTAN | 427 | SHOX2 | RAANCGAAAWTCGNTTY | 648 | RF7 |
| CTTTCCCMYAACACKNN | 225 | ZNF282 | NYAATTAN | 427 | UNCX | RRGWACANNNTGTWCYY | 649 | AKR1B1 |
| CYAATAAAAN | 226 | HOXD13 | NYMATAAAN | 428 | CDX1 | WAACCRCAAWAACCRCAN | 650 | RUNX2 |
| GAAASYGAAASY | 227 | IRF2 | NYMATAAAN | 428 | CDX2 | WAACCRCAAWAACCRCAN | 650 | RUNX3 |
| GAATGACACRGCGN | 228 | FOXB1 | NYMATTAN | 429 | HOXA7 | NNTCRCACNTANGTGYGANN | 651 | TBX19 |
| GAGCCTGGTACTGWGCCTGR | 229 | ZNF322 | NYMATTAN | 429 | NKX6-1 | YwTTkcKkTyyckgykky | 652 | AZF1 |

In some embodiments, binding sites are associated with more than one signaling molecule or complex of molecules.

Further, nonlimiting examples of such motifs or sites are given in Table 12.

TABLE 12
Motifs for Binding Sites or Genomic Signaling Centers

| | SEQ | | | SEQ | |
|---|---|---|---|---|---|
| Motif Sequence | ID | Complex_name | Motif Sequence | ID | Complex_name |
| ACCGGAAATGA | 653 | HOXB2:ETV1 | NRSCGGAAGNNGTAAAN | 944 | HOXD12:ELK1 |
| CCATAAATCA | 654 | PBX4:HOXA10 | NSCGGAARNCACGTGNN | 945 | ERF:SREBF2 |
| TAAACAGGATTA | 655 | FOXJ2:PITX1 | NSCGGAARNNNNNMATTAN | 946 | HOXB2:ETV7 |
| TGTCAATTA | 656 | MEIS1:DRGX | NSCGGAARNNNNNNMATTAN | 947 | HOXB2:ETV7 |
| ACCGGAAACAGCTGNN | 657 | TFAP4:FLI1 | NSCGGAARNNNNNNTCACACNN | 948 | ETV7:TBX21 |
| ACCGGAAATGAN | 658 | HOXB2:ETV4 | NSCGGAARNNNNNTCACACNN | 949 | ETV7:TBX21 |
| AGGTGTTAATKN | 659 | HOXB2:TBX3 | NSCGGAWNTTACGTAAN | 950 | ELK1:TEF |
| AGGTGTTGACAN | 660 | MEIS1:EOMES | NSMGGACGGAYNTCCKSN | 951 | ELK1:SPDEF |
| CTCGTAAAATGYN | 661 | TEAD4:HOXB13 | NSMGGACGGAYNTCCKSN | 951 | ERF:ETV7 |
| GAAAACCGAAAN | 662 | FLI1:FOXI1 | NSMGGACGGAYNTCCKSN | 951 | ETV2:ETV7 |
| GGAATGCGGAAGTN | 663 | TEAD4:ELF1 | NSMGGACGGAYNTCCKSN | 951 | FLI1:ETV7 |
| GGAATGTTAATTR | 664 | TEAD4:DRGX | NTAATKRSNMRTAAAN | 952 | HOXD12:HOXA3 |
| GTGCGGGCGGAAGTN | 665 | GCM1:ELF1 | NTAATNRSNYMRTAAAN | 953 | HOXD12:HOXA3 |
| TCACACCGGAWRY | 666 | ETV2:EOMES | NTGACRNNNNNNCACGTGN | 954 | MEIS1:MAX |
| TGTTGCCGGANNN | 667 | FOXO1:ELK1 | NTGTTGATRNGGGN | 955 | GCM1:FOXI1 |
| ACAAWRSNNNNNYMATTA | 668 | HOXB2:SOX15 | NTGTTGNCGGAARNN | 956 | FOXO1:ELF1 |
| ACAAWRSNNNNYMATTA | 669 | HOXB2:SOX15 | NTTTAYNNCCGGAARNN | 957 | HOXD12:ELK3 |
| ACAAWRSNNNYMATTA | 670 | HOXB2:SOX15 | NYMATTANNNNNACAATR | 958 | HOXB2:SOX15 |
| AGGTGTKAAGTCGTAAA | 671 | HOXD12:TBX21 | NYMATTANNNNNCAGCTGNN | 959 | HOXB2:NHLH1 |
| CAGCTGNNNNNNNNNNNTGCGGG | 672 | GCM1:FIGLA | NYMATTANNNNNNACAATR | 960 | HOXB2:SOX15 |
| CAGCTGNNNNNNNNNTGCGGG | 673 | GCM1:FIGLA | NYMATTANNNNNNCAGCTGNN | 961 | HOXB2:NHLH1 |
| CAGCTGNNNNNNNNTGCGGG | 674 | GCM1:FIGLA | NYMATTANNNNNNNCAGCTGNN | 962 | HOXB2:NHLH1 |
| CAGCTGNNNNNNNTGCGGG | 675 | GCM1:FIGLA | NYMATTANNNNNNNNGGAAGNN | 963 | HOXB2:ELF1 |
| | | | RAGGTSRNNNNNNNNNNNNNNNNCGGAAGY | 964 | ELK1:TBX21 |
| CCGGAANNNNNNCACGTG | 676 | ETV5:HES7 | N | | |
| GGAATGNNNNNYTAATTA | 677 | TEAD4:ALX4 | RCATTCCNNATCGAYN | 965 | TEAD4:ONECUT2 |
| GGATTANNNNNNNNNTGCGGG | 678 | GCM1:PITX1 | RCATTCCNNNNTAATKR | 966 | TEAD4:DLX3 |
| GGATTANNNNNNNTGCGGG | 679 | GCM1:PITX1 | RCATTCNNNNNNNNNNNNNNNNGCAACN | 967 | TEAD4:RFX5 |
| GGATTANNNNNNTGCGGG | 680 | GCM1:PITX1 | RCATTCNNNNNNNNNNNNNNNNNGCAACN | 968 | TEAD4:RFX5 |
| GGTGTGNNNNNCACGTG | 681 | CLOCK:TBX3 | RCATTCNNNNNTAATKR | 969 | TEAD4:DLX3 |
| TAATKNNNNNNNNAAGGTCA | 682 | HOXB2:ESRRB | RCATTCYNNNNCAAGGTCAN | 970 | TEAD4:ESRRB |
| TAATKRNGGATTA | 683 | HOXB2:PITX1 | RCATTCYNNNNTAATTR | 971 | TEAD4:DLX2 |
| TAATKRNNNNGCAAC | 684 | HOXB2:RFX5 | RCCGGAANNNNNATCGATN | 972 | ETV2:ONECUT2 |
| TAATKRNNNNGGATTA | 685 | HOXB2:PITX1 | RCCGGAANNNNNNATCGATN | 973 | ETV2:ONECUT2 |
| TAATKRNNNNGGATTA | 685 | PITX1:HOXA3 | RCCGGANNNNNNNNNNNACACCTN | 974 | ETV2:EOMES |
| TAATKRNNNNNGCAAC | 686 | HOXB2:RFX5 | RCCGGANNNNNNNNNNNACACCTN | 974 | ETV2:TBX21 |
| TAATKRNNNNNNAAGGTCA | 687 | HOXB2:ESRRB | RCCGGANNNNNNNNNNTAATCCN | 975 | ETV2:GSC2 |
| TAATKRNRNGGATTA | 688 | HOXB2:PITX1 | RCCGGANNNNNNNNNTAATCCN | 976 | ETV2:GSC2 |
| TAATTANNNNNNCACGTG | 689 | CLOCK:EVX1 | RCCGGANNNNNNNNTAATCCN | 977 | ETV2:GSC2 |
| TGACANNNNNTCATTA | 690 | MEIS1:EVX1 | RCCGGAWGTKKN | 978 | FOXO1:ETV1 |
| TGACANSNTAATTG | 691 | MEIS1:DLX3 | RCCGGAWGTKKN | 978 | FOXO1:ETV4 |
| TMACACCGGAAG | 692 | ERF:TBX21 | RCCGGAWGTKKW | 979 | FOXO1:ELK3 |
| TNTCACACCGGAAAT | 693 | ERF:EOMES | RGAATGCGGAARYNNNTCCN | 980 | TEAD4:ETV7 |
| ACCCGCANCCGGAAGN | 694 | GCM1:ELK3 | RGAATSCGGAAGYN | 981 | TEAD4:ELK1 |
| ACCGGANNTACGCNNNNNYR | 695 | ETV2:PAX5 | RGGTGTNNNNNNNNGAATGYN | 982 | TEAD4:EOMES |
| AGGTGNTAATKR | 696 | MGA:DLX3 | RGGTGYTAATWR | 983 | ALX4:TBX21 |
| AGGTGNTAATKW | 697 | MGA:EVX1 | RGTGTNNNAATATKNN | 984 | POU2F1:EOMES |
| AGGTGNTAATTR | 698 | MGA:DLX2 | RGWATGTTAATCCS | 985 | TEAD4:GSC2 |
| ATTTGCATNACAATRN | 699 | POU2F1:SOX2 | RKCACGTGNNNMCATATGKN | 986 | ARNTL:BHLHA15 |
| CACGTGNNNGCGGGY | 700 | GCM1:MAX | RKRNRGGCGGAARCGGAAGNN | 987 | GCM1:ELK1 |
| CACGTGNNNNNSRGGAARNN | 701 | ERF:CLOCK | RMATWCCGGAWRN | 988 | TEAD4:ELK1 |
| CACGTGNNNRGATTAN | 702 | ARNTL:PITX1 | RNCGGAANNNNNNNNNGCAACN | 989 | ETV2:RFX5 |
| CAGCTGNNNNNNCCCGCAY | 703 | GCM1:FIGLA | RNCGGANNTTGCGCAAN | 990 | FLI1:CEBPB |
| CYAATAAAATGYN | 704 | TEAD4:HOXB13 | RNCGGANNTTGCGCAAN | 990 | FLI1:CEBPD |
| GTMAACAGGAWRN | 705 | ETV5:FOXI1 | RRGTGTNNNNNNNNACAATRN | 991 | SOX6:TBX21 |
| GTMAATAAGGGYRN | 706 | GCM1:FOXO1 | RSCGGAAATRCM | 992 | TEAD4:ETV1 |
| TAATGNNNNNCGGAAGTN | 707 | HOXB2:ELK3 | RSCGGAAGNNGTAAAN | 993 | ELK1:HOXB13 |
| TAATKRCCGGAAGNN | 708 | HOXB2:ELK1 | RSCGGAAGTNGTAAAN | 994 | HOXD12:ETV1 |
| TAATKRNNNNGGAAGTN | 709 | HOXB2:ELF1 | RSCGGAANCACGTGN | 995 | ERF:MAX |
| TAATKRNNNNNGGAAGTN | 710 | HOXB2:ELF1 | RSCGGAANCACGTGN | 995 | FLI1:MAX |
| TAATTRNNNNCGGAARYN | 711 | ETV2:DLX3 | RSCGGAANNNNNNCACGTGNN | 996 | ETV2:HES7 |
| TGACAKNNNAACAATGN | 712 | MEIS1:SOX2 | RSCGGAANNNNNNNCACGTGNN | 997 | ETV2:HES7 |
| TGACANNNTAATKR | 713 | MEIS1:DLX2 | RSCGGAANNNNNNNTAATKR | 998 | ETV2:DLX3 |
| TGACASTAATKR | 714 | MEIS1:DRGX | RSCGGAANNNNNNRTCGATN | 999 | ERF:ONECUT2 |
| TGTTGANGCGGGN | 715 | GCMI:FOXI1 | RSCGGAANNNNNYAATTAN | 1000 | FLI1:DLX2 |
| TGTTGNCGGAWRN | 716 | ETV5:FOXI1 | RSCGGAANNRYMAACAN | 1001 | ETV2:FOXI1 |
| TGTTKMCGGAWRN | 717 | ERF:FOXI1 | RSCGGAANRWMAACAN | 1002 | ELK1:FOXI1 |
| TGTTKMCGGAWRNN | 718 | FLI1:FOXI1 | RSCGGAARCAGGTGN | 1003 | ETV5:FIGLA |
| TNRCACCGGAAGN | 719 | ELK1:EOMES | RSCGGAARYNNTAAAN | 1004 | HOXB13:ETV1 |
| TNRCACCGGAAGNN | 720 | ELK1:TBX21 | RSCGGANNCATATGK | 1005 | ETV2:BHLHA15 |
| TNRCACCGGAWN | 721 | ETV5:EOMES | RSCGGATGTTRTN | 1006 | FOXO1:ELK1 |
| TTACGTNNNNNNNNNCCGGAANN | 722 | ETV2:TEF | RSCGGAWNNNNNNACAATRN | 1007 | ETV2:SOX15 |
| ACCGGAARTNNNYAATTA | 723 | FLI1:DLX2 | RSCGGAWNNNNNNNACAATRN | 1008 | ETV2:SOX15 |
| AGGTGNGAARGGATTA | 724 | PITX1:TBX21 | RSCGGWAATKR | 1009 | ETV5:EVX1 |
| AGGTGTGNNNNATCRAT | 725 | CUX1:TBX3 | RSCGGWAATKR | 1009 | ETV5:HOXA2 |
| ANCCGGATNNNNNNMATTA | 726 | HOXB2:SPDEF | RSGTGNNNAACGK | 1010 | MYBL1:EOMES |
| ARGTGNNNNNNATAAA | 727 | HOXB13:TBX21 | RTAAACAYNRTAAAN | 1011 | FOXJ2:HOXB13 |
| ATCRATNNNNNNNNSYATTGTT | 728 | CUX1:SOX15 | RTAAACMGGAARYN | 1012 | ERF:FOXI1 |
| CACGTGNNNTAATKAT | 729 | CLOCK:EVX1 | RTAAACMGGAARYN | 1012 | FLI1:FOXI1 |
| CASSTGNNNNNNNNNNNTGCGGG | 730 | GCM1:FIGLA | RTAAATANGGGNN | 1013 | GCM1:FOXI1 |
| CASSTGNNNNNNNNNNTGCGGG | 731 | GCM1:FIGLA | RTAAATANGGGNN | 1013 | GCM2:FOXI1 |
| CASSTGNNNNNNNNTGCGGG | 732 | GCM1:FIGLA | RTCACGYSNCCGGAWN | 1014 | ETV2:SREBF2 |
| CASSTGNNNNNNNTGCGGG | 733 | GCM1:FIGLA | RTGCGGGNAGGTGNNN | 1015 | GCM2:EOMES |
| CYCATAAANNTGTCA | 734 | MEIS2:HOXA13 | RTGCGGGNNATCGATR | 1016 | GCM1:ONECUT2 |
| GGAATKNNCASSTG | 735 | TEAD4:FIGLA | RTGCGGGNNNNNNNCACGTGN | 1017 | GCM1:MAX |
| GGWATGNNNRTAAA | 736 | TEAD4:HOXA13 | RTGCGGGNNNTCGATR | 1018 | GCM1:ONECUT2 |
| GGWATGNNNRTAAA | 736 | TEAD4:HOXB13 | RTMAACAGGAAGTN | 1019 | FOXO1:ELK3 |
| GTTGCYNNNNNNNNNNNNCASSTG | 737 | RFX3:FIGLA | RTMAACAGGAAGTN | 1019 | FOXO1:FLI1 |
| GWMAACANNSYMRTAAA | 738 | FOXO1:HOXB13 | RTMAACAGGAARNN | 1020 | ERF:FOXO1 |
| GWMAACAYMRTAAA | 739 | FOXO1:HOXB13 | RTMAACAGGAWRN | 1021 | ETV5:FOXO1 |
| TAATCCNNNNCASSTG | 740 | PITX1:FIGLA | RTRCGGGTAATAAAN | 1022 | GCM2:HOM13 |
| TAATKAGGTGNKA | 741 | HOXA3:EOMES | RTRNNGGCGGAAGTN | 1023 | GCM1:ETV1 |
| TAATKANNGGATTA | 742 | HOXB2:PITX1 | RTRYGGGCGGAARKN | 1024 | GCMI1:ERG |
| TAATKRGGTGYM | 743 | HOXB2:EOMES | RWCACGTGNNCGGAANN | 1025 | ELK1:SREBF2 |
| TAATKRGGTGYM | 743 | HOXB2:TBX21 | RWMAACAGGNNNNNNTTCCNN | 1026 | FOXO1:ETV7 |
| TMACACCYAATA | 744 | CUX1:TBX21 | SGCGCCNNNNNNNNNNNNCAGCTGNN | 1027 | E2F1:NHLH1 |
| TTIWATNRNMAACA | 745 | FOXJ2:HOXB13 | SGCGCNNNNNCGGAAGN | 1028 | E2F1:ELK1 |
| AGGTGNTAATKWNNNNTN | 746 | MGA:EVX1 | SGCGCNNNNNNNNNNCGGAAGN | 1029 | E2F1:ELK1 |
| AGGTGTGNCSGTTR | 747 | MYBL1:TBX21 | SGCGCNNNNNNNNNNNCGGAAGN | 1030 | E2F1:ELK1 |
| AGGTGTNNNGSGGGN | 748 | GCM2:TBX21 | SGTCACGCNTCATTNN | 1031 | HOXB2:PAX9 |
| AGGTGTNRTRCGGGNN | 749 | GCM1:TBX21 | SYMATTANNNNNNRGCAACN | 1032 | HOXB2:RFX5 |
| ANGTGTGAATWCY | 750 | TEAD4:TBX21 | WTGMATAACAATR | 1033 | POU2F1:SOX15 |
| ANGTGTSNNATAAAN | 751 | HOXB13:EOMES | YNATTANRGGTGTGAN | 1034 | MGA:DLX3 |
| ATRCGGGYNNNNNYWTTGTNN | 752 | GCM2:SOX15 | NCACGTGNNNNNNCATWCC | 1035 | TEAD4:CLOCK |
| CASSTGNNCCCGCAY | 753 | GCM1:FIGLA | NCACGTGNNNNNNNCATWCC | 1036 | TEAD4:CLOCK |
| CASSTGNNCCGGAWRYN | 754 | ETV2:TCF3 | NCAGCTGNNNNNNNNTRCGGG | 1037 | GCMI:NHLH1 |
| CASSTGNRNNGGAAGNN | 755 | ETV5:TCF3 | NCAGCTGNNNNNNNTRCGGG | 1038 | GCMI:NHLH1 |
| CTCRTAAAWNNNNRMCGTTR | 756 | MYBL1:HOM13 | NCCGGAANNNNNNNMAPEC | 1039 | TEAD4:ELK3 |
| GGATTANNARGTGTKN | 757 | PITX1:EOMES | NCRCGTGNNNGGATTA | 1040 | PITX1:HES7 |
| GGATTANNNNATCRATN | 758 | CUX1:PITX1 | NGTGNNNNMATATKNNNACACC | 1041 | POU2F1:TBX21 |
| GGWATGNNNNNNCACGTGN | 759 | TEAD4:CLOCK | NNCRCGTGNNNNGGATTA | 1042 | PITX1:HES7 |
| GGWATGNNNNNNNCACGTGN | 760 | TEAD4:CLOCK | NNMATTAGTCACGCWTSRNTG | 1043 | HOXB2:PAX1 |
| GGWATGNRAGGTGNNR | 761 | TEAD4:EOMES | NNMATTAGTCACGCWTSRNTG | 1043 | HOXB2:PAX5 |
| TAATKNNNNGNNNNNNCTTCCNN | 762 | HOXB2:ETV7 | NNMATTAGTCACGCWTSRNTG | 1043 | HOXB2:PAX9 |
| TAATKRSCGGAWGN | 763 | ETV5:DRGX | NNNTATGCAGYGTKA | 1044 | POU2F1:EOMES |
| TGACAGNWAATCRATR | 764 | MEIS1:ONECUT2 | NNTCCCGCNNNCCNNNGGC | 1045 | TFAP2C:E2F8 |
| TGTTKATRCGGGN | 765 | GCM2:FOXI1 | NSCCNNNRGGCANNNNYMATTA | 1046 | TFAP2C:DLX3 |
| TGTTKMCGGAWRTN | 766 | ETV2:FOXI1 | NSCGGACGGAWATCCGSNT | 1047 | ETV2:SPDEF |
| TMACACMGGAARN | 767 | ETV2:TBX21 | NSCGGANNNNNGGMTTA | 1048 | ERF:PITX1 |
| TTGCGYAANNSCGGAAGN | 768 | CEBPG:ELF1 | NSCGGANNNNNNGGMTTA | 1049 | ERF:PITX1 |
| ATCRATNNNNYCRTAAA | 769 | CUX1:HOXA13 | NTRNGGGNNNCACGTG | 1050 | GCM2:MAX |
| ATRATYANNNNCACGTG | 770 | CLOCK:EVX1 | NTTTATNRNTMAACA | 1051 | FOXJ2:HOXB13 |
| GGWATGYGTMAACA | 771 | TEAD4:FOXI1 | RCATWCCNNNNNNNNYNNTAAA | 1052 | TEAD4:HOM13 |
| TMACACYYMRTWAA | 772 | HOXC10:EOMES | RCATWCNNNNGGATTA | 1053 | TEAD4:PITX1 |
| TMRCACCTCRTWAA | 773 | HOXD12:EOMES | RCATWCNNNNNGGATTA | 1054 | TEAD4:PITX1 |
| ARGTGNNANNNMWTAAAN | 774 | HOXB13:TBX21 | RCATWCNNNNNNAGGTCA | 1055 | TEAD4:ESRRB |
| ARGTGTKANTTTATNN | 775 | HOXB13:TBX21 | RCATWCYNNNNNTAATCC | 1056 | TEAD4:PITX1 |
| ARGTGTKRNNNNNRGWATGY | 776 | TEAD4:TBX21 | RCATWCYNNNNTAATCC | 1057 | TEAD4:PITX1 |
| CYCRTAAATWCCN | 777 | TEAD4:HOXA13 | RGGTGTKANNNNGGATTA | 1058 | PITX1:EOMES |
| | | | RMATATKCNNNNNNNNNNNNNNNNRWMAAC | 1059 | POU2F1:FOXO6 |
| GTMAACANNNNNATCRATN | 778 | CUX1:FOXO1 | A | | |
| TGMATATKCANNNNNTAATKR | 779 | POU2F1:DLX2 | RMATATKCNNNNNNNNNNNNNNNRWMAACA | 1060 | POU2F1:FOXO6 |
| TGMATATKCANNNNTAATKR | 780 | POU2F1:DLX2 | RMATATKCNNNNNNNNNNNNNRWMAACA | 1061 | POU2F1:FOXO6 |
| TNSCCNNNGGSNNNCACGTGN | 781 | TFAP2C:MAX | RNCGGAWGTMATTA | 1062 | ETV5:HOXA2 |
| TNSCCNNNGGSNNNNCACGTGN | 782 | TFAP2C:MAX | RSCGGAANNNNNNNNCATWCC | 1063 | TEAD4:ERG |
| TNSCCNNNGGSNNNNNNNNNNNNNCACGTG | | | | | |
| N | 783 | TFAP2C:MAX | RSCGGAANTSRCGTGA | 1064 | ELK1:SREBF2 |
| TNSCCNNNGGSNNNNNNNNNNNNNNCACGT | | | | | |
| GN | 784 | TFAP2C:MAX | RSCGGAASNGRTCGATA | 1065 | ELK1:ONECUT2 |
| TMRCACYTMATWAA | 785 | HOXC10:TBX21 | RSCGGWAATKNNNNNNNNMATTA | 1066 | ETV5:HOXA2 |
| ANGTGNNANNNNNNNNNNNCNNMGGAWNN | 786 | ELK1:TBX21 | RSCGGWANNNNYMATTA | 1067 | ELK1:EVX1 |
| ARGTGTNNNAATATKYNNNCRCNN | 787 | POU2F1:EOMES | RSMGGAWGYAATTA | 1068 | ETV5:DRGX |
| ATRSGGGNNNNTTRCGYAAN | 788 | GCM1:CEBPB | RTAAACWNATWAAA | 1069 | FOXJ2:HOXB13 |
| GGWATGYNNNNCRCGYGY | 789 | TEAD4:HES7 | RTGNKGGCGGAWG | 1070 | GCM1:SPDEF |
| GGWATGYNNNNNNCRCGYGY | 790 | TEAD4:HES7 | RTRCGGGNNGATTA | 1071 | GCM2:PITX1 |
| TRGYAACNNNNCASSTGNN | 791 | RFX3:FIGLA | RTRCGGGNNNNTTACGTAA | 1072 | GCM2:TEF |
| AGGYGYGANNNNNNNNNNNNNNNTCRTWAA | 792 | HOXD12:EOMES | RWMAACASYMRTWAAA | 1073 | FOXO1:HOXB13 |
| NNCGTAAAATTA | 793 | HOXB2:HOXB13 | SCCNNNGGCNNYAATTA | 1074 | TFAP2C:DRGX |
| NRMATATACCAATAAA | 794 | POU2F1:HOXB13 | SYAATTANWGGTGYGA | 1075 | MGA:DLX2 |
| NTCGTAAAATGC | 795 | TEAD4:HOXB13 | WTWTGCATANNTAATTA | 1076 | POU2F1:DLX2 |
| NTCGTAAATCA | 796 | PBX4:HOXA10 | YNRCACSTCGTWAA | 1077 | HOXD12:TBX21 |
| RCCGGAAGTAATTA | 797 | ELK1:HOXA1 | NAACGGNNATYGANN | 1078 | MYBL1:ONECUT2 |
| YNNRTAAATCAATCA | 798 | PBX4:HOXA10 | NATCGATNNNNNGCCTNNGGSNN | 1079 | TFAP2C:ONECUT2 |
| NCCGGATATGCAN | 799 | POU2F1:ELK1 | NATCGATNNNNNNNGCCTNNGGSNN | 1080 | TFAP2C:ONECUT2 |
| NCCGGATATGCAN | 799 | POU2F1:ETV1 | NATCGATNNNNNNNNGCCTNNGGSNN | 1081 | TFAP2C:ONECUT2 |
| NCCGGATATGCAN | 799 | POU2F1:ETV4 | NATCRATNNNNNNNAACAATRS | 1082 | CUX1:SOX15 |
| NGATGATGCAATNN | 800 | ATF4:CEBPD | NATCRATNNNNNNNNAACAATRS | 1083 | CUX1:SOX15 |
| NGCAGCTGCCGGAWRYN | 801 | ETV2:NHLH1 | NATTTRCNNNACAATRN | 1084 | POU2F1:SOX2 |
| NGCCACGCAAYN | 802 | CEBPG:CREB3L1 | NCACGTGNNYAACSGNN | 1085 | MYBL1:MAX |
| NMATGACACGCGCCMNN | 803 | E2F3:FOXO6 | NCAGGTGNGWATGYN | 1086 | TEAD4:TCF3 |
| NMCCGGAACCGTTR | 804 | MYBL1:ELF1 | NCAGSTGNNNNNNGTAATKR | 1087 | TFAP4:DLX3 |
| NNCAGCTGCCGGAWRYN | 805 | ERF:NHLH1 | NCAGSTGNNNNNNNGTAATKR | 1088 | TFAP4:DLX3 |
| NNGAAAACCGAANM | 806 | FOXO1:ELF1 | NCASSTGKNNNNNNNNCACGTGN | 1089 | CLOCK:FIGLA |
| NNMATCACATAAAN | 807 | HOXB2:HOXB13 | NCASSTGKNNNNNNNNNCACGTGN | 1090 | CLOCK:FIGLA |
| NNMCACCGCGCCCMN | 808 | E2F3:FOXI1 | NCCGGAAGYNNCNTAGCAACS | 1091 | ETV2:RFX5 |
| NNNRTAAATCACACNN | 809 | HOXB13:TBX3 | NCCGGAANNNNNNCACGYGNN | 1092 | ERF:HES7 |
| NTGCCGGAAGTN | 810 | MEIS1:ELF1 | NCCGGAANTNRTAAAN | 1093 | FLI1:HOXB13 |
| NWAAACAGGAAGNN | 811 | FOXJ2:ELF1 | NCCGGANNCASSTGY | 1094 | FLI1:TCF3 |
| RCCGGAAATRSY | 812 | TEAD4:FLI1 | NGATAASNNNRGWATGY | 1095 | TEAD4:GATA3 |
| RCCGGATGTTKWN | 813 | ETV2:FOX06 | NGCCTNNGGSNNCGGAAGYN | 1096 | TFAP2C:ELK1 |
| RCCGGATGTTKWY | 814 | FLI1:FOXI1 | NGGTGTGNNGGCGCSNNNNCRMN | 1097 | E2F3:TBX21 |
| RGAATGCGGAAGTN | 815 | TEAD4:SPIB | NNCACGTGNNNNRGMTTAN | 1098 | ARNTL:PITX1 |
| RGAATGCGGAAGTR | 816 | TEAD4:ELF1 | NNCAGCTGNNNNNNNNNATYGATN | 1099 | CUX1:NHLH1 |
| RGAATGCGGATN | 817 | TEAD4:SPDEF | NNCGGAWGYMATTAN | 1100 | FLI1:DRGX |
| RGGTGTTAATKN | 818 | HOXB2:EOMES | NNGGAMGGATKTCCGSN | 1101 | ETV5:ETV7 |
| RMAGAAAACCGAANN | 819 | FOXJ2:ELF1 | NNGTMAATANGGGYR | 1102 | GCM1:FOXI1 |
| RNCGGATGTTKWN | 820 | ETV5:FOXO1 | NNGYGNNNNNNNNWAACAACACNN | 1103 | FOXJ3:TBX21 |
| RNMTGATGCAATN | 821 | ATF4:TEF | NNGYGYSACATTCCN | 1104 | TEAD4:EOMES |
| RSCGGAAGTAATAAAN | 822 | HOXD12:ELK1 | NNMGGAARTGCKGGN | 1105 | GCM1:ELF1 |
| RSCGGAAGTAATAAAN | 822 | HOXD12:ELK3 | NNMRTAAANTMACACNN | 1106 | HOXB13:EOMES |
| RSCGGAAGTAATAAAN | 822 | HOXD12:ETV1 | NNNMGGAANNNNNTCCNNNRCAACN | 1107 | RFX3:ETV7 |
| RSCGGAAGTAATAAAN | 822 | HOXD12:ETV4 | NNNRTAAANNTNACACYN | 1108 | HOXB13:TBX3 |
| RSCGGAAGTCGTAAAN | 823 | HOXD12:ELK3 | NNNRTAAAWATYGAYY | 1109 | HOXB13:ONECUT2 |
| RSCGGAAGTCGTAAAN | 823 | HOXD12:ETV4 | NNRGYAACNTCACGTGAY | 1110 | RFX3:SREBF2 |
| RSCGGATGTKKN | 824 | ELK1:FOXI1 | NRGTGTNANNNNNNNNNNNNNCCGGAANN | 1111 | ERF:TBX21 |
| RSCGGATGTTGN | 825 | ETV5:FOXI1 | NRGYAACNNNCATATGKN | 1112 | RFX3:BHLHA15 |
| RSCGGATGTTKW | 826 | ETV2:FOXI1 | NRTCRATANCGGAARYN | 1113 | ETV2:ONECUT2 |
| RSCGGATGTTKWN | 827 | ERF:FOXI1 | NRTMAACMGGAARYN | 1114 | ETV2:FOXI1 |
| RSCGGTAATKR | 828 | ELK1:HOXA3 | NSCCNNNRGGCANNNNNNTAATKR | 1115 | TFAP2C:DLX3 |
| RTGCGGGCGGAAGTN | 829 | GCM1:ELK1 | NSCCNNNRGGCANNNNNTAATKR | 1116 | TFAP2C:DLX3 |
| RTGCGGGCGGAAGTN | 829 | GCM1:ELK3 | NTCACACMNNNATCRATN | 1117 | CUX1:TBX21 |
| RTGCGGGCGGAAGTR | 830 | GCM1:ETV4 | NTGACAGNTAATCRATAN | 1118 | MEIS2:ONECUT2 |
| RTGCGGGTAATAAAN | 831 | GCM1:HOXB13 | NTNNNGGCGGAAGNNNTTCCNNN | 1119 | GCM1:ETV7 |
| RWMAACAGGAAGTN | 832 | FOXO1:ELF1 | NTRNGGGCGGAAGNNNTTCCNNN | 1120 | GCM1:ETV7 |
| RWMAACAGGAARNN | 833 | FOXO1:ETV4 | NTRYGGGNNNTGGCGGGARN | 1121 | GCM2:E2F8 |
| RWYAACAGGAAGYN | 834 | FOXO1:ELK1 | NYMRTAAANATYGATN | 1122 | CUX1:HOM13 |
| SGCGCTAATTKN | 835 | E2F3:DRGX | RATTCCNNNNNNNNNNCASSTGN | 1123 | TEAD4:FIGLA |
| WAACAACACW | 836 | FOXJ3:TBX21 | RCATWCCNNNNNNNNNNNNRWGCGTGACN | 1124 | TEAD4:PAX5 |
| WMSCGGATGTKNW | 837 | FOXO1:SPDEF | RCATWCCNNNNNNTTTAYNNN | 1125 | TEAD4:HOM13 |
| NCACCTGNNNNNMATTA | 838 | HOXB2:TCF3 | RCCGGAANNNNNNRATCRATN | 1126 | ETV2:ONECUT2 |
| NCACGTGNNGGATTA | 839 | PITX1:HES7 | RCCGGAARCASSTGNN | 1127 | TFAP4:ETV4 |
| NCAGGTGNNNNNMATTA | 840 | HOXB2:TCF3 | RCCGGANRNNCGGWATKN | 1128 | TEAD4:ELK1 |
| NCCCGCANNMATAAA | 841 | GCM1:HOXB13 | RGGTGNTAAKCCN | 1129 | MGA:PITX1 |
| NCCCGCANNNMRTAAA | 842 | GCM1:HOXB13 | RGGTGNTAATNNNNNNNNNCASYNN | 1130 | ALX4:TBX21 |
| NCCGGAAGNNNNNNYMATTA | 843 | ETV2:HOXA2 | RGWATGYTAATKN | 1131 | TEAD4:DRGX |
| NCCGGAAGTMATTA | 844 | ETV2:HOXA2 | RGWATGYTAATKR | 1132 | TEAD4:DLX2 |
| NNATCATNGTAAA | 845 | HOXB2:HOXB13 | RNCGGAAGNNNNNCASSTGN | 1133 | ETV5:TCF3 |
| NNATGAYGCAAT | 846 | CEBPG:ATF4 | RNCGGAANYNRIWAAN | 1134 | ELK1:HOXB13 |
| NNMAACAYNRTAAA | 847 | HOXB13:Fox | RNGTGNNNNNNNNNNNNNRCRCCGGAWSN | 1135 | ETV5:EOMES |
| NTAATCCNNWMAACA | 848 | FOXJ2:PITX1 | RRGTGTKNNNNNNNNNNNNNNCMGGANNN | 1136 | ERF:EOMES |
| NYNATMAATCA | 849 | PBX4:HOM1 | RRTCRATNNNCGGAARYN | 1137 | ETV2:ONECUT2 |
| RCATTCCNNNNNNCACGTG | 850 | TEAD4:MAX | RSCGGAANCAGSTGNN | 1138 | TFAP4:ETV1 |
| RCATTCCNNNNNNNCACGTG | 851 | TEAD4:MAX | RSCGGAANCASCTGNN | 1139 | ERF:FIGLA |
| RCATTCCNNNYAATTA | 852 | TEAD4:DLX3 | RSCGGAANCASSTGN | 1140 | FLI1:FIGLA |
| RCATTCCNNNYMATTA | 853 | TEAD4:DLX3 | RSCGGAANNNNNGGMTTAN | 1141 | ETV2:PITX1 |
| RCATTCNNNNNNCATTA | 854 | TEAD4:HOXA2 | RSCGGAASNGRTCGATAN | 1142 | ELK1:ONECUT2 |
| RCATTCNNNNNNCATTA | 854 | TEAD4:HOXA3 | RSCGGANNTTGCGYAAN | 1143 | ETV2:CEBPD |
| RCATTCNNNNNTAATCC | 855 | TEAD4:PITX1 | RSCGGAWRCASSTGN | 1144 | TFAP4:FLI1 |
| RCATTCYNNNCAATTA | 856 | TEAD4:DLX2 | RSCGGWAATNNNNATTAN | 1145 | ETV5:EVX1 |
| RCATTCYNNNNCAATTA | 857 | TEAD4:DLX2 | RSCGGWAATNNNNNATTAN | 1146 | ETV5:EVX1 |
| RCATTCYNNNNNCATTA | 858 | TEAD4:HOXA3 | RSCGGWAATNNNYATTAN | 1147 | ETV2:EVX1 |
| RCCGGAAATRCC | 859 | TEAD4:ETV4 | RSMGGAANTTGCGYAAN | 1148 | ERF:CEBPD |
| RCCGGAANNNNNNYAATTA | 860 | ETV2:DLX2 | RSMGGAARTNNTAAAN | 1149 | HOXB13:ELK1 |
| RCCGGAANNNNNYAATTA | 861 | ETV2:DLX2 | RTAAACANNNNNATCRATN | 1150 | CUX1:FOXI1 |
| RGAATGCGGAWRT | 862 | TEAD4:SPDEF | RTAAACANNNNNATCRATN | 1150 | CUX1:FOXO6 |
| RGAATGCNNRTAAA | 863 | TEAD4:HOXB13 | RTMAACATRNGGGN | 1151 | GCM1:FOXI1 |
| RGAATGYGTGA | 864 | TEAD4:EOMES | RTMAATAMGGGYRN | 1152 | GCM1:FOXO1 |
| RGAATGYNNACGTG | 865 | TEAD4:MAX | RTMRCGTGACGGAWGN | 1153 | ETV2:SREBF2 |
| RGGTGTNNNNNNNNNYATTGT | 866 | SOX6:TBX21 | RTRCGGGNNNNNNNYWTTGTNN | 1154 | GCM2:SOX15 |
| RKRNGGGNNATAAA | 867 | GCM1:HOM13 | RTRCGGGNNNNNNTAATKR | 1155 | GCM2:DLX3 |
| RNCGGAANNAAACA | 868 | ETV2:FOXI1 | RTRCGGGNNNNNTAATKR | 1156 | GCM2:DLX3 |
| RRAATGCARTAAA | 869 | TEAD4:HOXB13 | RTRCGGGNNNNNTAATTR | 1157 | GCM2:DLX2 |
| RSCGGAAATRCC | 870 | TEAD4:ERG | RTRCGGGNNNNRNACAAWN | 1158 | GCM2:SOX15 |
| RSCGGAAGTMRTTA | 871 | HOXB2:ELK1 | RTRCGGGNNNRNACAAWN | 1159 | GCM2:SOX15 |
| RSCGGAAGTMRTTA | 871 | HOXB2:ELK3 | RTRCGGGSGATTAN | 1160 | GCM2:PITX1 |
| RSCGGAANNNNGGATTA | 872 | ETV2:GSC2 | RTRNGGGTNNTAAAN | 1161 | GCM1:HOXB13 |
| RSCGGAANNNNNGGATTA | 873 | ETV2:GSC2 | RTRSGGGNAATTAN | 1162 | GCM2:DRGX |
| RSCGGAANNNNNNNYWTTGT | 874 | ETV2:SOX15 | RTRSGGGNNAATTAN | 1163 | GCM2:DRGX |
| RSCGGAANNNNNNRTCGAT | 875 | FLI1:ONECUT2 | RTRSGGGNNNATTGTKY | 1164 | GCM1:SOX2 |
| RSCGGAANNNNNNYAATTA | 876 | ETV2:DLX3 | RTRSGGGNNTAATKR | 1165 | GCM1:HOXA2 |
| RSCGGAANNNNNNYWTTGT | 877 | ETV2:SOX15 | RTRSKGGCGGANNNNNATCCNNN | 1166 | GCM1:SPDEF |
| RSCGGAANNNNNYAATTA | 878 | ERF:DLX3 | RTRSKGGCGGANNNNNNATCCNNN | 1167 | GCM1:SPDEF |
| RSCGGAANNNNNYAATTA | 878 | ETV2:DLX3 | SYMRTAAANATYGATN | 1168 | CUX1:HOXB13 |
| RSCGGAANNNNNYAATTA | 878 | ETV5:DLX2 | SYMRTAAANNNNCASSTGN | 1169 | HOXD12:FIGLA |
| RSCGGAANNNNNYMATTA | 879 | ERF:DLX2 | WGTTKMCGGAWRTN | 1170 | ELK1:FOXI1 |
| RSCGGAANNNNYAATTA | 880 | ERF:DLX3 | YNATTAGTCACGCWTSRNTR | 1171 | HOXA3:PAX5 |
| RSCGGAANYAATTA | 881 | ETV2:DRGX | NCASSTGNNNNNNNNRTRCGGG | 1172 | GCM2:FIGLA |
| RSCGGAANYNRTAAA | 882 | FLI1:HOXB13 | NGGTGTGNNNGGCGCSNNNCRC | 1173 | E2F1:EOMES |
| RSCGGAARYAATTA | 883 | FLI1:DLX2 | NNCGGANGNNNTWAA | 1174 | ETV5:HOXB13 |
| RSCGGAWNNNNNNNYMATTA | 884 | ERF:HOXA3 | NNCGGAWGTNRTWAA | 1175 | ETV5:HOXA13 |
| RSCGGAWRYATTA | 885 | ETV2:DRGX | NTATKCAGYGTNA | 1176 | POU2F1:EOMES |
| SMGGAAGTMRTTA | 886 | HOXB2:ELF1 | NTATKCAGYGTNA | 1176 | POU2F1:TBX21 |
| SYMRTAAANCTGTCA | 887 | MEIS1:HOXB13 | RATCRATNNNNNNNNNNRGYAAC | 1177 | CUX1:RFX5 |
| SYNRTAAANNTGTCA | 888 | MEIS1:HOM13 | RATCRATNNNNNNNNNRGYAAC | 1178 | CUX1:RFX5 |
| YAACGGNNNNNNNNNNCACGTG | 889 | MYBL1:MAX | RCATWCCNNNNNNNNNNNNNNRTMAACA | 1179 | TEAD4:FOXI1 |
| YAACGGNNNNNNNNNNNCACGTG | 890 | MYBL1:MAX | RCATWCCNNNNNNNNNNNNNRTMAACA | 1180 | TEAD4:FOXI1 |
| YRATTANNNNNNNCACGTG | 891 | CLOCK:EVX1 | RCATWCCNNNNNNNNNNNNRTMAACA | 1181 | TEAD4:FOXI1 |
| NACAATRNNNNGAATGY | 892 | TEAD4:SOX15 | RCATWCCNNNNNNNNNNNRTMAACA | 1182 | TEAD4:FOXI1 |
| NACAATRNNNNNGAATGY | 893 | TEAD4:SOX15 | RCATWCYNNNNTAATYRNATTA | 1183 | TEAD4:ALX4 |
| NACTTCCGGYNNNNGCAACSN | 894 | ETV2:RFX5 | RSCGGAACYACGCWYSANTG | 1184 | ELK1:PAX1 |
| NAGGTGNTAATKR | 895 | HOXB2:TBX21 | RSCGGAACYACGCWYSANTG | 1184 | ELK1:PAX5 |
| NCACGTGNNNNNCATATGN | 896 | CLOCK:BHLHA15 | RSCGGAACYACGCWYSANTG | 1184 | ELK1:PAX9 |
| NCACGTGNNNNNSCGGAWRN | 897 | ETV5:CLOCK | RSCGGAANNAGGYGYNA | 1185 | ELK1:EOMES |
| NCAGCTGNNNNNCACGTGN | 898 | TFAP4:MAX | RSCGGAANNRTMAAYA | 1186 | ELK1:FOXI1 |
| NCAGCTGNNNNNNNCACGTGN | 899 | TFAP4:MAX | RSCGGAANRTMAAYA | 1187 | ELK1:FOXI1 |
| NCCGGAAGTYRTAAAN | 900 | ETV2:HOXB13 | RTRCGGGNNCASSTG | 1188 | GCM2:FIGLA |
| NCCGGAANCATATGN | 901 | FLI1:BHLHA15 | RWMAACAYCRTWAA | 1189 | FOXO1:HOXA10 |
| NCCGGAANNNNNNCAGCTGNN | 902 | ETV2:NHLH1 | SCCNNNNGGCATNGTWAA | 1190 | TFAP2C:HOXB13 |
| NCCGGAANYATAAAN | 903 | ETV2:HOXB13 | NATYGATNNNNNNNNNGCCTNNGGSNN | 1191 | TFAP2C:ONECUT2 |
| NCCGGWNNNNNNNNNNNSCATTAN | 904 | ELK1:HOXA3 | NCASSTGNNNNNNNCSGTTR | 1192 | MYBL1:FIGLA |
| NGGTGTGNNGGCGCSN | 905 | E2F3:TBX21 | NCASSTGNNNNNNNNCSGTTR | 1193 | MYBL1:FIGLA |
| NGGTGTGNNNGGCGCSN | 906 | E2F3:TBX21 | NCASSTGNNNNNNNNNCSGTTR | 1194 | MYBL1:FIGLA |
| NGGTGTNNNNNNNNNNNNNNNCCGGAWNNN | 907 | ETV2:EOMES | NCASSTGNNNNNNYAACSGYN | 1195 | MYBL1:FIGLA |
| NMATGACACNGCGCCMNN | 908 | E2F3:FOXO6 | NCASSTGNNNNNYAACSGYN | 1196 | MYBL1:FIGLA |
| NNACAATNNNNNNGAATGY | 909 | TEAD4:SOX6 | NCCATAYWNGGWNNNNATCRATN | 1197 | CUX1:SRF |
| NNARAAAACCGAAWMN | 910 | FOXJ3:ELF1 | NGGTGTGNNNNNTMATTWGCRN | 1198 | HOXB2:TBX21 |
| NNATGAYGCAAYN | 911 | ATF4:CEBPB | NRGCAACNNNCRCGYGNN | 1199 | RFX3:HES7 |
| NNCACGTGACMGGAARNN | 912 | ERF:SREBF2 | NRGYAACNNNNNCCIWWWNNGGN | 1200 | RFX3:SRF |
| NNCACGTGNNNGCGGGYN | 913 | GCM1:MAX | NSCGGANNTTRCGYAAN | 1201 | ETV5:CEBPD |
| NNCACGTGNNNNCCGGAANN | 914 | ERF:HES7 | NTTRCGYAANNNNNNGGAATGY | 1202 | TEAD4:CEBPB |
| NNCACGTGNNNNCCGGAANN | 914 | ETV5:HES7 | RCATTCCNNCRCGYGYN | 1203 | TEAD4:HES7 |
| NNCACGTGNNNNNCCGGAANN | 915 | ERF:HES7 | RCATTCCNNNNNCRCGYGYN | 1204 | TEAD4:HES7 |
| NNCACGTGNNNNNCCGGAWRY | 916 | ETV2:CLOCK | RGGTGNGANNNNNNNNTNNCACCGGAAGY | 1205 | ELK1:EOMES |
| NNCACGTGNNNNNCGGAWRY | 917 | ETV2:HES7 | RGGTGTNNNGGCGSNNTNNCRSNN | 1206 | E2F1:EOMES |
| NNCACGTGNNNNNNNNNNRTGCGGGYRN | 918 | GCM1:MAX | RGTGTKRNNNNNNNNNNNCNCMGGAARN | 1207 | ETV2:TBX21 |
| NNCAGCTGNNNNNNCACGTGNN | 919 | CLOCK:NHLH1 | RGWATSCGGATGNNNNTCCKNNN | 1208 | TEAD4:SPDEF |
| NNCAGCTGNNNNNNNNATCGATN | 920 | CUX1:NHLH1 | RRCRCGYGYNNNNSGCGCSN | 1209 | E2F1:HES7 |
| NNCAGCTGNNNNNNNTAATTN | 921 | TFAP4:DLX3 | RSCGGAANNRTMAAYAN | 1210 | ELK1:FOXI1 |
| NNCAGCTGNNNNTAATKR | 922 | TFAP4:DLX3 | RSCGGAWNTTRCGYAAN | 1211 | ETV2:TEF |
| NNCAGGTGNNNNNMCGGAARYN | 923 | ETV2:FIGLA | RTGNKGGCGGAWGNNNNNTCCGGNN | 1212 | GCM1:SPDEF |
| NNCGGAANCAGGTGNN | 924 | ETV2:FIGLA | RTGNKGGCGGAWGNNNNTCCGGNN | 1213 | GCM1:SPDEF |
| NNGATTANNNATGCAWNNN | 925 | POU2F1:GSC2 | RTRCGGGNNNNNNATCRATN | 1214 | GCM2:ONECUT2 |
| NNGTCACGCNNCATTAN | 926 | HOXB2:PAX5 | RTRNKGGTNNNGCACGYGNN | 1215 | GCM2:HES7 |
| NNGTCACGNNTCATTNN | 927 | HOXB2:PAX1 | SCCNNNGGSNNNGGAAGNNNTTCCNN | 1216 | TFAP2C:ETV7 |
| NNMGGAARNNRTAAAN | 928 | ERF:HOXB13 | SRCRCGYGSYNNNNSGCGCSN | 1217 | E2F1:HES7 |
| NNNACGANNNNNNTCGTNNN | 929 | HOXD12:EOMES | WNGTGYKAMATWCY | 1218 | TEAD4:EOMES |
| NNNCGGGNNNGGTGTNN | 930 | GCM2:TBX21 | MTRSGGGNNNNNNTTRCGYAAN | 1219 | GCM1:CEBPB |
| NNNGMATAACAAWRRN | 931 | POU2F1:SOX15 | MTRSGGGNNNNNTTRCGYAAN | 1220 | GCM1:CEBPB |
| NNNNGCGCSNNNNNCACGTGNN | 932 | E2F3:HES7 | NGGTGTNNNNNNNNNNNNNCACNTNNTWAN | 1221 | HOXD12:TBX21 |
| NNNRTAAANCTGTN | 933 | HOXB13:MEIS1 | NGYGYTAAYNNNNNNTNACACNN | 1222 | ALX4:EOMES |
| NNNTTCCGSNNNNGCAACNN | 934 | ETV2:RFX5 | NNCRCGYGNNNNNNNSCCNNNGGS | 1223 | TFAP2C:HES7 |
| NNRNGGGCGGAARTN | 935 | GCM2:ELK1 | NNCRCGYGNNNNNNSCCNNNGGS | 1224 | TFAP2C:HES7 |
| NNSGCGCSNNNATCGAYN | 936 | E2F3:ONECUT2 | NNGYGNNNNGGCGCSNNNNCRCNN | 1225 | E2F3:EOMES |
| NNSGCGCSNNNNATCGAYN | 937 | E2F3:ONECUT2 | NNNTATGCNNNGYGANNNNNNNNNNNCRCN | 1226 | POU2F1:EOMES |
| NNSMGGACGGAYNTCCKSNN | 938 | ELK1:ETV7 | NNSYGNCNAACNNNNNNCACRCNN | 1227 | MYBL1:EOMES |
| NNYMATTANNNNNNNGGAAGNN | 939 | HOXB2:ELF1 | NTTRCGYAANNNNNNNNRGWATGY | 1228 | TEAD4:CEBPD |
| NRCAGCTGNNNNNCACGTGNN | 940 | CLOCK:NHLH1 | NTTRCGYAANNNNNNNRGWATGY | 1229 | TEAD4:CEBPD |
| NRCATTCNNNNNTAATTRN | 941 | TEAD4:DLX2 | NTTRCGYAANNNNNNRGWATGY | 1230 | TEAD4:CEBPD |
| NRCATTCNNNNYAATTN | 942 | TEAD4:DLX2 | RGGTGWKNNNNNNNNNTNNCRTRNGGGN | 1231 | GCM1:TBX21 |
| NRCCCRNNCGGAAGNN | 943 | GCM1:ELF1 | RGWATGYNNTTRCGYAAN | 1232 | TEAD4:CEBPD |
| | | | NGYGNNAMATWCYNNTMRCRCN | 1233 | TEAD4:EOMES |

It has further been determined that certain patterns are found in the binding motifs. A list of such patterns for complexes (Tables 13 and 14) and single molecules (Tables 15 and 16) are shown below.

TABLE 13
Gene Complex Motif Patterns

| | Number of each pattern |
|---|---|
| Pattern | in Gene Complex Table |
| AB | 11 |
| ABA | 27 |
| ABAB | 29 |
| ABABA | 25 |
| ABABAB | 23 |
| ABABABA | 5 |
| ABABABAB | 11 |
| ABABABABA | 1 |
| ABABABABAB | 6 |
| ABABABABABA | 1 |

TABLE 14
Gene Complex Motif Patterns

| | Number of each pattern |
|---|---|
| Pattern | in Gene Complex Table |
| BA | 6 |
| BAB | 46 |
| BABA | 58 |
| BABAB | 155 |
| BABABA | 45 |
| BABABAB | 95 |
| BABABABA | 22 |
| BABABABAB | 28 |
| BABABABABAB | 14 |
| BABABABABABABABAB | 1 |

TABLE 15
Single Gene Motif Patterns

| | Number of each pattern |
|---|---|
| Pattern | in Single Gene Table |
| AB | 42 |
| ABA | 97 |
| ABAB | 37 |
| ABABA | 40 |
| ABABAB | 4 |
| ABABABA | 28 |
| ABABABAB | 4 |
| ABABABABA | 4 |
| ABABABABABA | 1 |

TABLE 16
Single Gene Motif Patterns

| | Number of each pattern |
|---|---|
| Pattern | in Single Gene Table |
| BA | 34 |
| BAB | 186 |
| BABA | 40 |
| BABAB | 120 |
| BABABA | 15 |
| BABABAB | 47 |
| BABABABA | 8 |
| BABABABAB | 8 |
| BABABABABA | 1 |
| BABABABABAB | 2 |

Table 17 shows motifs which may be targeted by biomolecule complexes (such as transcription factors or signaling molecules or chromatin remodeling molecules, in any combination) acting at GSCs. “A” represents a normal nucleotide region, and “B” represents a variable nucleotide, gap, or spacer region.

TABLE 17
Complex motif patterns starting with A

| | Number of | | | | | | | | | | | |
|---|---|---|---|---|---|---|---|---|---|---|---|---|
| | each pattern | | | | | | | | | | | |
| | in gene | | | | | | | | | | | |
| | complex | Number of nucleotides/variables | | | | | | | | | | |
| Pattern | table | A1 | B1 | A2 | B2 | A3 | B3 | A4 | B4 | A5 | B5 | A6 |
| AB | 11 | 10-14 | 1-3 | | | | | | | | | |
| ABA | 27 | 1, 4-6 | 1, 3, 5-11 | 4-7, 10, 13 | | | | | | | | |
| ABAB | 29 | 1, 2, 4-8 | 1-7, 9 | 4-9 | 1-4, 7 | | | | | | | |
| ABABA | 25 | 1-7 | 1-4, 13 | 1-6 | 1-4, 6-8, 10, 11 | 1, 2, 4-6 | | | | | | |
| ABABAB | 23 | 1-7 | 1-5 | 1-4, 8 | 1-4, 6-8 | 1-6 | 1-4 | | | | | |
| ABABABA | 5 | 1-3 | 1, 2 | 2-4, 6 | 1, 4, 5 | 1, 2 | 2, 4, 6 | | | | | |
| ABABABAB | 11 | 1, 2 | 1, 2 | 1-4 | 1-3, 5, 8 | 1-3, 5 | 1, 4, 5, 14, 15 | 2-6 | 1, 2 | | | |
| ABABABABA | 1 | 1 | 2 | 3 | 1 | 1 | 1 | 2 | 1 | 2 | | |
| ABABABABAB | 6 | 1, 2 | 1, 2 | 1, 3, 4 | 1-5, 7 | 1-3, 5 | 1, 4, 5, 11 | 1-3 | 1-3 | 1-3 | 1-3 | |
| ABABABABABA | 1 | 3 | 1 | 1 | 1 | 2 | 15 | 1 | 1 | 1 | 1 | 2 |

Table 18 is an additional table of motifs which may be targeted by biomolecule complexes (such as transcription factors or signaling molecules or chromatin remodeling molecules, in any combination) acting at GSCs. “A” represents a normal nucleotide region, and “B” represents a variable nucleotide, gap, or spacer region.

TABLE 18
Complex motif patterns starting with B

| | Number of | | | | | |
|---|---|---|---|---|---|---|
| | each pattern | | | | | |
| | in gene | | | | | |
| | complex | Number of nucleotides/variables | | | | |
| Pattern | table | B1 | A1 | B2 | A2 | B3 |
| BA | 6 | 1-4 | 10-13 | | | |
| BAB | 46 | 1-4 | 7-13 | 1-4 | | |
| BABA | 58 | 1-4 | 2-9 | 1-11 | 1-6 | |
| BABAB | 155 | 1-5 | 1-8, 10 | 1-13, 15-18 | 1-11 | 1-4 |
| BABABA | 45 | 1-3 | 1-8, 11 | 1-8 | 1-6 | 1-6, 10, 11, 16, 18, 19 |
| BABABAB | 95 | 1-4 | 1-7, 11 | 1-10, 14, 15 | 1-6, 8, 9 | 1-10, 13, 14 |
| BABABABA | 22 | 1-3 | 1-6 | 1-4 | 1-5 | 1-5, 10-15 |
| BABABABAB | 28 | 1-2 | 1-6 | 1-3, 13 | 1-4, 6 | 1, 3-9 |
| BABABABABA | 14 | 1-4 | 1, 2, 4, 5 | 1-3, 11, 13 | 1-3 | 1-6 |
| BABABABABAB | 1 | 1 | 1 | 1 | 1 | 2 |

| | Number of nucleotides/variables | | | | | | | | | | | |
|---|---|---|---|---|---|---|---|---|---|---|---|---|
| Pattern | A3 | B4 | A4 | B5 | A5 | B6 | A6 | B7 | A7 | B8 | A8 | B9 |
| BA | | | | | | | | | | | | |
| BAB | | | | | | | | | | | | |
| BABA | | | | | | | | | | | | |
| BABAB | | | | | | | | | | | | |
| BABABA | 1-6, 8 | | | | | | | | | | | |
| BABABAB | 1-8 | 1-4 | | | | | | | | | | |
| BABABABA | 1, 2, 4 | 1-3 | 1-4 | | | | | | | | | |
| BABABABAB | 1-6 | 1-7 | 1-6, 9 | 1-4 | | | | | | | | |
| BABABABABA | 1-3, 5 | 1, 2, 5-9, 11 | 1-3 | 1-3 | 1-4 | 1-2 | | | | | | |
| BABABABABAB | 1 | 1 | 2 | 1 | 1 | 3 | 1 | 2 | 1 | 1 | 1 | 1 |

Table 19 is a table of motifs which may be targeted by single biomolecules such as transcription factors or signaling molecules or chromatin remodeling molecules acting at GSCs. “A” represents a normal nucleotide region, and “B” represents a variable nucleotide, gap, or spacer region.

TABLE 19
Single motif patterns starting with A

| | Number of | | | | | | | | | | | |
|---|---|---|---|---|---|---|---|---|---|---|---|---|
| | each pattern | | | | | | | | | | | |
| | in gene | | | | | | | | | | | |
| | complex | Number of nucleotides/variables | | | | | | | | | | |
| Pattern | table | A1 | B1 | A2 | B2 | A3 | B3 | A4 | B4 | A5 | B5 | A6 |
| AB | 42 | 6-13, 15, 16 | 1-3 | | | | | | | | | |
| ABA | 97 | 1-9, 11, 12, 16 | 1-5, 10, 11 | 1-10, 12, 13, 15 | | | | | | | | |
| ABAB | 37 | 1-9, 13 | 1-3 | 1-8, 12 | 1-3 | | | | | | | |
| ABABA | 40 | 1-7 | 1-4 | 1-7, 9, 10, 13, 14 | 1-5 | 1-6, 8, 9 | | | | | | |
| ABABAB | 4 | 1, 2, 4, 9 | 1, 2 | 1, 3, 4 | 1, 2, 6 | 1, 2, 6, 7 | 1, 2 | | | | | |
| ABABABA | 28 | 1-6, 10 | 1, 2 | 1-4, 6, 8 | 1-6 | 1-6, 8 | 1-3 | 1-3, 5-7, 9 | | | | |
| ABABABAB | 4 | 3, 4, 7 | 1, 2 | 1, 3, 4 | 1, 2 | 2, 3 | 1, 2 | 1, 2, 4 | 1, 4 | | | |
| ABABABABA | 4 | 1, 3, 4 | 1, 2 | 1, 6 | 1, 4 | 1-3 | 1 | 1-3 | 1, 2 | 1-4 | | |
| ABABABABABA | 1 | 1 | 1 | 5 | 1 | 1 | 2 | 2 | 1 | 3 | 1 | 2 |

Table 20 is a table of motifs which may be targeted by single biomolecules such as transcription factors or signaling molecules or chromatin remodeling molecules acting at GSCs. “A” represents a normal nucleotide region, and “B” represents a variable nucleotide, gap, or spacer region.

TABLE 20
Single motif patterns starting with B

| | Number of | | | | | | | | | | | |
|---|---|---|---|---|---|---|---|---|---|---|---|---|
| | each pattern | | | | | | | | | | | |
| | in gene | | | | | | | | | | | |
| | complex | Number of nucleotides/variables | | | | | | | | | | |
| Pattern | table | B1 | A1 | B2 | A2 | B3 | A3 | B4 | A4 | B5 | A5 | B6 |
| BA | 34 | 1-4, 6 | 4-13 | | | | | | | | | |
| BAB | 186 | 1-5 | 4-12, 14-16 | 1-4 | | | | | | | | |
| BABA | 40 | 1-3 | 1, 2, 4-8, 10-12 | 1-5 | 1-10 | | | | | | | |
| BABAB | 120 | 1-4 | 1-8, 10-12 | 1-6 | 1-11 | 1-4 | | | | | | |
| BABABA | 15 | 1, 2 | 1-7, 9 | 1-3 | 1-5, 9 | 1, 2, 4 | 1-3, 5-7, 9 | | | | | |
| BABABAB | 47 | 1-4 | 1-7 | 1-3, 5 | 1-6, 9, 11 | 1-4 | 1-6 | 1-3 | | | | |
| BABABABA | 8 | 1, 2 | 1, 2, 7 | 1, 2 | 1, 3, 4 | 1, 2 | 1-5 | 1 | 1, 3, 9 | | | |
| BABABABAB | 8 | 1, 2 | 1, 2, 4, 6 | 1 | 1, 3, 5 | 1-3 | 1-4 | 1 | 1, 2, 3, 6 | 1, 2 | | |
| BABABABABA | 1 | 1 | 1 | 1 | 4 | 1 | 8 | 1 | 3 | 1 | 4 | |
| BABABABABAB | 2 | 2 | 2 | 1 | 1, 3 | 1, 2 | 1, 2 | 1, 2 | 1, 3 | 1 | 1, 2 | 2, 4 |

In the Motif Tables above, certain designators are used according to the IUPAC nucleotide code. This code is shown in Table 21.

TABLE 21
IUPAC Nucleotide Code

| IUPAC code | Base | IUPAC code | Base |
|---|---|---|---|
| A | Adenine | K | G or T |
| C | Cytosine | M | A or C |
| G | Guanine | B | C or G or T |
| T (or U) | Thymine (or Uracil) | D | A or G or T |
| R | A or G | H | A or C or T |
| Y | C or T | V | A or C or G |
| S | G or C | N | Any base |
| W | A or T | “.” or “-” | gap |

### Cohesin and CTCF Associated Loops and Anchor Site/Regions

CTCF interactions link sites on the same chromosome forming loops, which are generally less than 1 Mb in length. Transcription occurs both within and outside the loops, but the nature of this transcription differs between the two regions. Studies show that enhancer-associated transcription is more prominent within the loops. Thus, the insulator state is enriched specifically at the CTCF loop anchors. CTCF loops thus either enclose gene poor regions, with a tendency for genes to be centered within the loops or leave out gene dense regions outside the CTCF loops. FIG. 2A and FIG. 2B compare the linear to the 3-dimensional (3D) conformation of the loops.

CTCF loops exhibit reduced exon density relative to their flanking regions. Gene ontology analysis reveals that genes located within CTCF loops are enriched for response to stimuli and for extracellular, plasma membrane, and vesicle cellular localizations. On the other hand, genes present within the flanking regions just outside the loops exhibit an expression pattern similar to housekeeping genes i.e. these genes are on average more highly expressed than the loop-enclosed genes, are less cell-line specific in their expression pattern, and have less variation in their expression levels across cell lines. See Oti et al., BMC Genomics, 17:252, 2016, which is hereby incorporated by reference herein in its entirety.

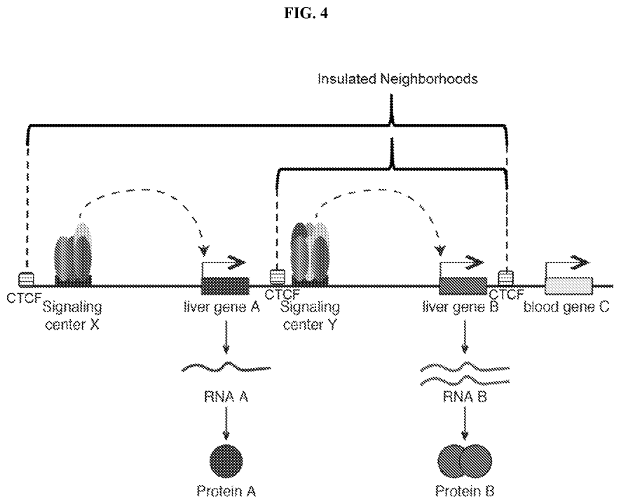

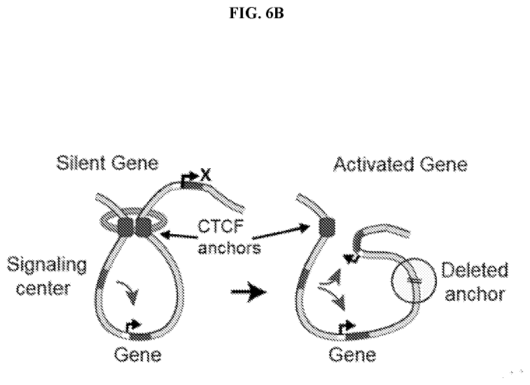

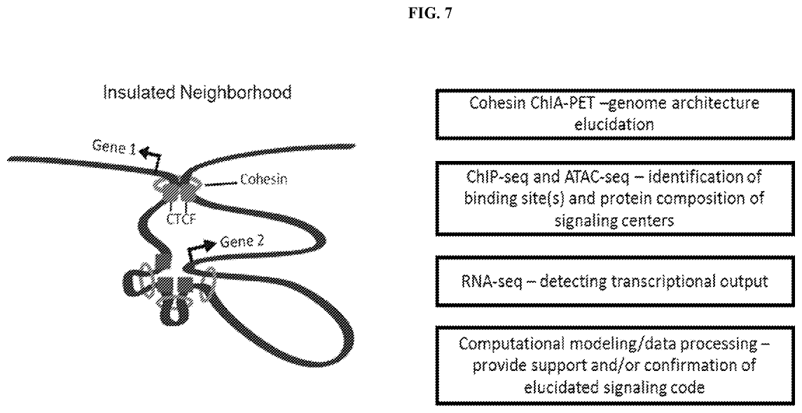

Anchor regions are binding sites for CTCF that influence conformation of an insulated neighborhood. FIG. 4 shows the relationship between the CTCF anchor regions and gene transcription. Deletion of anchor sites may result in activation of genes that are usually transcriptionally silent, thereby resulting in a disease phenotype. In fact, somatic mutations are common in loop anchor sites of oncogene-associated insulated neighborhoods. The CTCF DNA-binding motif of the loop anchor region has been observed to be the most altered human transcription-factor binding sequence of cancer cells. See, Hnisz et al., Cell 167, Nov. 17, 2016, which is hereby incorporated by reference here in its entirety. FIG. 6B is an illustration of the differences between conformation between a transcriptionally silent gene and an activated gene due to deletions of a deleted CTCF anchor region.

Anchor regions have been observed to be largely maintained during cell development, and are especially conserved in the germline of humans and primates. In fact, the DNA sequence of anchor regions are more conserved in CTCF anchor regions than at CTCF binding sites that are not part of an insulated neighborhood. Therefore, cohesin may be used as a target for ChIA-PET to identify locations of both.

Cohesin also becomes associated with CTCF-bound regions of the genome, and some of these cohesin-associated CTCF sites facilitate gene activation while others may function as insulators. See, Dixon et al., Nature, 485(7398):376-80, 2012; Parelho et al., Cell, 132(3):422-33, 2008; Phillips-Cremins and Corces, Molecular Cell, 50(4):461-74, 2013); Seitan et al., Genome Research, 23(12):2066-77, 2013; Wendt et al., Nature, 451(7180):796-801, 2008), which are hereby incorporated by reference in their entireties. Cohesin and CTCF are associated with large loop substructures within TADs, and cohesin and Mediator are associated with smaller loop structures that form within CTCF-bounded regions. See, de Wit et al., Nature, 501(7466):227-31, 2013; Cremins et al., Cell, 153(6):1281-95, 2013; Sofueva et al., EMBO, 32(24):3119-29, 2013, which are hereby incorporated by reference in their entireties. FIG. 3B shows nested gene loops within an insulated neighborhood. In some embodiments, cohesin and CTCF associated loops and anchor sites/regions may be targeted to after or elucidate GSNs.

### Genetic Variants

Genetic variations within IN boundary sites are known to contribute to disease by disrupting protein binding on chromosomes, such as described in Hnisz et al., Cell 167, Nov. 17, 2016, which is hereby incorporated by reference in its entirety. Variations of the sequence of CTCF anchor regions of IN boundary sites that interfere with formation of INs are observed to result in dysregulation of gene activation and repression. CTCF malfunctions caused by various genetic and epigenetic mechanisms may lead to pathogenesis. Moreover, genetic variations within binding sites of signaling molecules may disrupt genomic signaling centers by altering the binding profile. In certain embodiments, genetic variations within binding sites of signaling molecules may disrupt an ODSC. Therefore, in some embodiments, itis beneficial to after any one or more GSNs associated with such variant-driven etiology in order to effect one or more positive treatment outcomes.

### Single Nucleotide Polymorphisms (SNPs)

94.2% of SNPs occur in non-coding regions, which include enhancer regions. See, Hnisz et al., Cell, 155, 934-947, 2013; which is hereby incorporated by reference herein in its entirety. In some embodiments, SNPs are altered in order to study and/or after the signaling from one or more GSNs. Most disease associated SNPs are located in the proximity of GSCs. In some embodiments, SNPs are altered to restore a GSC occurring in the genome without the SNP. Alternatively, SNPs are altered to change at least one binding site of a signaling molecule to change the binding profile of a GSC. In certain embodiments, SNPs are altered to change at least one binding site of a signaling molecule to change the binding profile of an ODSC.

### Signaling Molecules

Signaling molecules/signaling proteins include any protein that functions in cellular signaling pathways, whether canonical or the GSN pathways defined herein or capable of being defined using the methods described herein. Transcription factors are a subset of signaling molecules/signaling proteins. Certain combinations of signaling and master transcription factors associate to an enhancer region to influence expression of a gene. Master transcriptin factors direct transcription factors in specific tissues. For example, in blood, GATA transcription factors are master transcription factors that direct TCF7L2 of the Wnt cellular signaling pathway. In the liver, HNF4A is a master transcription factor to direct SMAD in lineage tissues and patterns.

Transcriptional regulation allows controlling how often a given gene is transcribed. Transcription factors alter the rate at which transcripts are produced by making conditions for transcription initiation more or less favorable. A transcription factor selectively alters a signaling pathway which in turn affects the genes controlled by a GSC. GSCs are transcriptional regulators. In some embodiments, signaling molecules/signaling proteins may be used or targeted in order to elucidate or after the signaling of GSNs of the present invention. For example, the signaling proteins of an ODSC are targeted.

Table 22 provides a list of signaling molecules/signaling proteins including those which act as transcription factors (TF) and/or chromatin remodeling factors (CR) that function in various cellular signaling pathways.

Lengthy table referenced here
US20210254056A1-20210819-T00001
Please refer to the end of the specification for access instructions.

The methods described herein may be used to inhibit or activate the expression of one or more signaling molecules/signaling proteins associated with the RSR of the primary neighborhood gene encoded within an IN. The methods may thus after the signaling signature of one or more primary neighborhood genes which are differentially expressed upon treatment with the therapeutic agent compared to an untreated control.

Various embodiments of the transcripts encoding the signaling proteins of Table 22 contain internal stop codons. These internal stop codons result in translation of multiple polypeptides. In one embodiment, a polypeptide that is a fragment of the signaling proteins taught in Table 22 may have signaling properties.

As a non-limiting example, the polypeptide may be a fragment such as SEQ ID NO: 32606 and SEQ ID NO: 32607 from SEQ ID NO: 5047. As a non-limiting example, the polypeptide may be a fragment such as SEQ ID NO: 32608 and SEQ ID NO: 32609 from SEQ ID NO: 5048. As a non-limiting example, the polypeptide may be a fragment such as SEQ ID NO: 32610 and SEQ ID NO: 32611 from SEQ ID NO: 5049. As a non-limiting example, the polypeptide may be a fragment such as SEQ ID NO: 32612-32613 from SEQ ID NO: 5050. As a non-limiting example, the polypeptide may be a fragment such as SEQ ID NO: 32614 and SEQ ID NO: 32615 from SEQ ID NO: 5051. As a non-limiting example, the polypeptide may be a fragment such as SEQ ID NO: 32616 and SEQ ID NO: 32617 from SEQ ID NO: 5053. As a non-limiting example, the polypeptide may be a fragment such as SEQ ID NO: 32618 and SEQ ID NO: 32619 from SEQ ID NO: 5054. As a non-limiting example, the polypeptide may be a fragment such as SEQ ID NO: 32620 and SEQ ID NO: 32621 from SEQ ID NO: 5055. As a non-limiting example, the polypeptide may be a fragment such as SEQ ID NO: 32622-32626 from SEQ ID NO: 18908.

Transcription Factors

Transcription factors generally regulate gene expression by binding to enhancers and recruiting coactivators and RNA polymerase II to target genes. See, Whyte et al., Cell, 153(2): 307-319, 2013, which is hereby incorporated by reference herein in its entirety. Transcription factors bind “enhancers” to stimulate cell-specific transcriptional program by binding regulatory elements distributed throughout the genome. The context-specific combination of transcription factors and signaling molecules/signaling proteins and the region of the genome to which they are bound may be referred to as a GSC. In certain embodiments of a GSC, the presence of (i) at least 2 signaling proteins, and (ii) a H3K27 chemical modification or independently at least one of a bromodomain-containing protein (Brd), a transcriptional coactivator, and at least two master transcription factors define an ODSC.

There are about 1800 known transcription factors in the human genome. There are epitopes on the DNA of the chromosomes that provide binding sites for proteins or nucleic acid molecules such as ribosomal RNA complexes. Master transcription factors direct a combination of signaling transcription factors through cell signaling above and DNA below. These characteristics allow for determination of the location of the next GSC. In some embodiments, transcription factors may be used or targeted, to after or elucidate the GSNs of the present invention.

Master Transcription Factors

Master transcription factors bind and establish cell-type specific enhancers. Master transcription factors recruit additional signaling proteins and signaling transcription factors, to enhancers to form GSCs. An atlas of candidate master TFs for 233 human cell types and tissues is described in D'Alessio et al., Stem Cell Reports 5, 763-775 (2015), which is hereby incorporated by reference in its entirety. In some embodiments, master transcription factors may be used or targeted, to after or elucidate the GSNs of the present invention.

### Signaling Transcription Factors

Signaling transcription factors are transcription factors, such as homeoproteins, that travel between cells as they contain protein domains that allow them to do the so. Homeoproteins such as Engrailed, Hoxa5, Hoxb4, Hoxc8, Emx1, Emx2, Otx2 and Pax6 are able to act as signaling transcription factors. The homeoprotein Engrailed possesses internalization and secretion signals that are believed to be present in other homeoproteins as well. This property allows homeoproteins to act as signaling molecules/signaling proteins in addition to being transcription factors. Homeoproteins lack characterized extracellular functions leading to the perception that their paracrine targets are intracellular. The ability of homeoproteins to regulate transcription and, in some cases, translation is most likely to affect paracrine action. See Prochiantz and Joliot, Nature Reviews Molecular Cell Biology, 2003. In some embodiments, signaling transcription factors may be used or targeted, to after or elucidate the GSNs of the present invention.

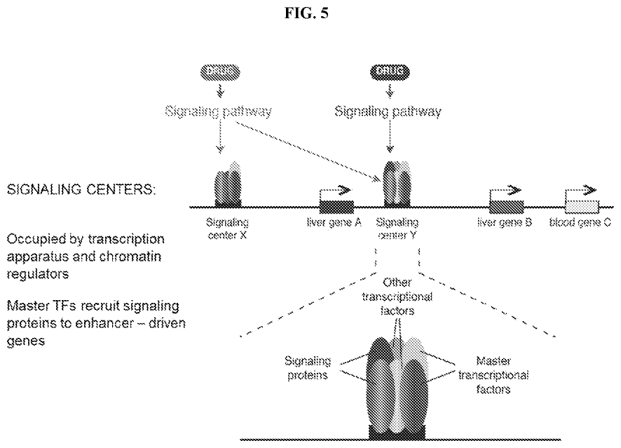

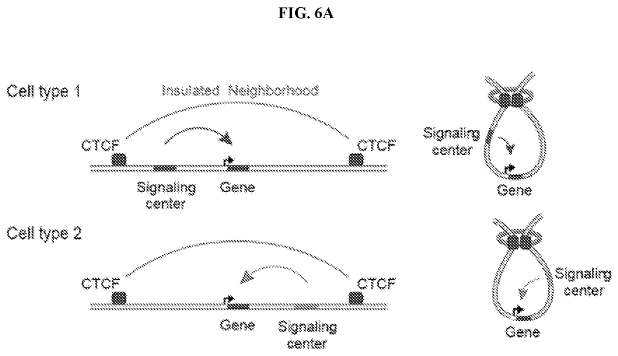

FIG. 5 illustrates the components of a GSC; including transcriptional factors, signaling proteins, and chromatin regulators.

### Chromatin Modifications

Chromatin remodeling is regulated by over a thousand proteins that are associated with histone modification. See, Ji et al., PNAS, 112(12):3841-3846(2015), which is hereby incorporated by reference in its entirety. Chromatin regulators are specific sets of proteins associated with genomic regions marked with modified histones. For example, histones may be modified at certain lysine residues: H3K20me3, H3K27ac, H3K4me1, H3K4me3, H3K79me2, H3K36me3, H3K9me3, and H3K9me2. Certain histone modifications mark regions of the genome that are available for binding by signaling molecules/signaling proteins. For example, previous studies have observed that active enhancer regions include nucleosomes with H3K27ac, and active promoters include nucleosomes with H3K27ac. Further, transcribed genes include nucleosomes with H3K79me2. ChIP-MS may be performed identify chromatin regulator proteins associated with specific histone modification. ChIP-seq with antibodies specific to certain modified histones may also be used to identify regions of the genome that are bound by signaling molecules/signaling proteins. In some embodiments, chromatin modifying enzymes or proteins may be used or targeted, to after or elucidate the GSNs of the present invention.

RNAs Derived from Regulatory Sequence Regions

Many active regulatory sequence regions (RSRs), such as regions from enhancers, GSCs, and promoters of protein-coding genes, are known to produce non-coding RNAs. Transcripts produced at or in the vicinity of active regulatory sequence regions have been implicated in transcription regulation of nearby genes. Recent reports have demonstrated that enhancer-associated RNAs (eRNAs) are strong indicators of enhancer activity (See Li et al., Nat Rev Genet. 2016 April; 17(4):207-23, which is hereby incorporated by reference in its entirety). Further, non-coding RNAs from active RSRs have been shown to be involved in facilitating the binding of transcription factors to these regions (Sigova et al., Science. 2015 Nov. 20; 350(6263):978-81, which is hereby incorporated by reference in its entirety). This suggests that such RNAs may be important for the assembly of GSCs and regulation of neighborhood genes.

In some embodiments, RNAs derived from regulatory sequence regions may be an enhancer-associated RNA (eRNA). In some embodiments, RNAs derived from RSRs may be a promoter-associated RNA, including but not limited to, a promoter upstream transcript (PROMPT), a promoter-associated long RNA (PALR), and a promoter-associated small RNA (PASR). In further embodiments, RNAs derived from RSRs may include but are not limited to transcription start sites (TSS)-associated RNAs (TSSa-RNAs), transcription initiation RNAs (tiRNAs), and terminator-associated small RNAs (TASRs).

In some embodiments, RNAs derived from RSRs may be long non-coding RNAs (IncRNAs) (i.e., >200 nucleotides). In some embodiments, RNAs derived from RSRs may be intermediate non-coding RNAs (i.e., about 50 to 200 nucleotides). In some embodiments, RNAs derived from RSRs may be short non-coding RNAs (i.e., about 20 to 50 nucleotides).

In some embodiments, eRNAs that may be modulated by methods and compounds described herein may be characterized by one or more of the following features: (1) transcribed from regions with high levels of monomethylation on lysine 4 of histone 3 (H3K4me1) and low levels of trimethylation on lysine 4 of histone 3 (H3K4me3); (2) transcribed from genomic regions with high levels of acetylation on lysine 27 of histone 3 (H3K27ac); (3) transcribed from genomic regions with low levels of trimethylation on lysine 36 of histone 3 (H3K36me3); (4) transcribed from genomic regions enriched for RNA polymerase II (Pol II); (5) transcribed from genomic regions enriched for transcriptional co-regulators, such as the p300 co-activator; (6) transcribed from genomic regions with low density of CpG island; (7) their transcription is initiated from Pol II-binding sites and elongated bidirectionally; (8) evolutionarily conserved DNA sequences encoding eRNAs; (9) short half-life; (10) reduced levels of splicing and polyadenylation, (11) dynamically regulated upon signaling; (12) positively correlated to levels of nearby mRNA expression; (13) extremely high tissue specificity; (14) preferentially nuclear and chromatin-bound; and/or (15) degraded by the exosome.

Exemplary eRNAs include those described in Djebali et al., Nature. 2012 Sep. 6; 489(7414) (for example, Supplementary data file for FIG. 5a) and Andersson et al., Nature. 2014 Mar. 27; 507(7493):455-461 (for example, Supplementary Tables S3, S12, S13, S15, and 16), which are herein incorporated by reference in their entireties.

In some embodiments, promoter-associated RNAs that may be modulated by methods or compounds described herein may be characterized by one or more of the following features: (1) transcribed from regions with high levels of H3K4me1 and low to medium levels of H3K4me3; (2) transcribed from genomic regions with high levels of H3K27ac; (3) transcribed from genomic regions with no or low levels of H3K36me3; (4) transcribed from genomic regions enriched for RNA polymerase II (Pol II); (5) transcribed from genomic regions with high density of CpG island; (6) their transcription is initiated from Pol II-binding sites and elongated in the opposite direction from the sense strand (that is, mRNAs) or bidirectionally; (7) short half-life; (8) reduced levels of splicing and polyadenylation; (9) preferentially nuclear and chromatin-bound; and/or (10) degraded by the exosome.

In some embodiments, methods and compositions described herein may be used to modulate RNAs derived from RSRs to after or elucidate the GSNs of the present invention. In some embodiments, methods and compounds described herein may be used to inhibit the production and/or function of an RNA derived from RSRs. In some embodiments, a hybridizing oligonucleotide such as an siRNA or an antisense oligonucleotide may be used to inhibit the activity of the RNA of interest via RNA interference (RNA), or RNase H-mediated cleavage, or physically block binding of various signaling molecules to the RNA. Exemplary hybridizing oligonucleotide may include those described in U.S. Pat. No. 9,518,261 and WO 2014/040742, which are hereby incorporated by reference in their entireties. The hybridizing oligonucleotide may be provided as a chemically modified or unmodified RNA, DNA, locked nucleic acids (LNA), or a combination of RNA and DNA, a nucleic acid vector encoding the hybridizing oligonucleotide, or a virus carrying such vector. In other embodiments, genome editing tools such as CRISPR/Cas9 may be used to delete specific DNA elements in the regulatory sequence regions that control the transcription of the RNA or degrade the RNA itself. In other embodiments, genome editing tools such as a catalytically inactive CRISPR/Cas9 may be used to bind to specific elements in the regulatory sequence regions and block the transcription of the RNA of interest. In further embodiments, bromodomain and extra-terminal domain (BET) inhibitors (e.g., JQ1, I-BET) may be used to reduce RNA transcription through inhibition of histone acetylation by BET protein Brd4.

In alternative embodiments, methods and compounds described herein may be used to increase the production and/or function of an RNA derived from regulatory sequence regions. In some embodiments, an exogenous synthetic RNA that mimic the RNA of interest may be introduced into the cell. The synthetic RNA may be provided as an RNA, a nucleic acid vector encoding the RNA, or a virus carrying such vector. In other embodiments, genome editing tools such as CRISPR/Cas9 may be used to tether an exogenous synthetic RNA to specific sites in the RSRs. Such RNA may be fused to the guide RNA of the CRISPR/Cas9 complex.

### Perturbation of Genomic Systems

Behavior of one or more components of the GSNs, INs, GSCs, and/or ODSC described herein may be altered by contacting the systems containing such features with a perturbation stimulus. Potential stimuli include exogenous biomolecules such as small molecules, antibodies, proteins, peptides, lipids, fats, nucleic acids, and the like or environmental stimuli such as radiation, pH, temperature, ionic strength, sound, light and the like. In some embodiments, these stimuli selectively target an ODSC comprising a nucleic acid sequence SEQ ID NOs: 32,627-71,281. In some embodiments, contacting a genomic system with a perturbation stimulus results in an alteration of the genome architecture, such as gene looping. Alternatively, in some embodiments, the binding profile of the GSC is altered by the perturbation stimuli.

The present invention serves, not only as a discovery tool for the elucidation of better defined GSNs and consequently a better understanding of biological systems. The present invention allows, for the first time, the ability to properly define gene signaling at the gene level in a manner which allows the prediction, a priori, of potential treatment outcomes, the identification of novel gene targets which may have never been implicated in the pathology of a disease or condition path and/or the resolution, reduction or removal of one or more treatment liabilities associated with new or known drugs such as toxicity, poor haft-life, poor bioavailability, lack of or loss of efficacy or pharmacokinetic or pharmacodynamic risks.

Treatment of disease by altering gene expression of canonical cellular signaling pathways has been shown to be effective. Even small changes in gene expression may have a significant impact on disease. For example, changes in GSCs affecting signaling pathways related to cell suicide suppression are associated with disease. The present invention, by elucidating a more definitive set of connectivities of the GSNs provides a fine-tuned mechanism to address disease, including genetic diseases. In some embodiments, a method of treating a disease may include modifying a GSC that is involved with a gene associated with that disease. For example, a method of treating a disease may include modifying an ODSC that is involved with a gene associated with that disease. Such genes may not presently be associated with the disease except as is elucidated using the methods described herein.

### Perturbation Stimuli

Below are provided listings of various stimuli which may be used to perturb GSCs of INs or GSNs. The stimuli herein also may be used to perturb ODSCs. These include small molecules, known drugs, biologicals, gene therapy products, and other products. Any of the compounds listed below can be used to as a perturbation to study changes in gene expression and to define GSNs associated with each stimulus.

In some embodiments, the perturbation stimulus binds at least a portion of a GSC. In some embodiments, the perturbation stimulus binds at least a portion of an ODSC comprising a nucleic acid sequence of one of SEQ ID NOs: 32,627-71,281. In some embodiments, the perturbation stimulus is formulated as a pharmaceutical composition with a pharmaceutically acceptable excipient.

### Small Molecules

In certain embodiments, a stimulus that perturbs a GSC is a small molecule drug selected from: (−)-Epigallocatechin 3-gallate, (−)-phenserine, (+)-calanolide A, (R)-folitixorin, (R)-mequitazine, (S)-pantoprazole sodium, [11C]DASB, [11C]-raclopride, [18F] FDG, [18F] HX4, 1-(2-chloroethyl)-3-cyclohexyl-1-nitrosourea, 1,2-decanediol, 11,11-di-deutero-ethyl linoleate, 11C-PBR-28, 123I-iometopane, 124I-CLR-1404, 131I-M1BG, 131-Iodine, 13-cis-retinoic acid, 13C-labeled methacetin, 13N-ammonia, 1400W94, 17 beta-estradiol, 17-alpha hydroxyprogesterone caproate, 17-beta-estradiol, 17beta-estradiol valerate, 17-hydroxysteroid dehydrogenase inhibitors, 18F-EF5, 18F-FDG, 2 L polyethylene glycol, 25-dihydroxy-vitamin D3, 25-OH vitamin D, 2-chloroprocaine, 2-deoxyglucose, 2-Hydroxypropyl-Beta-Cyclodextrin, 2MD, 2-methoxyestradiol, 4-aminopyridine, 4-aminosalicylic acid, 4-FEC, 4-hydroxytamoxifen, 5-aminolevulinic acid, 5-aminosalicylic acid, 5-aracytine, 5-fluorouracil (5-FU), 5-hydroxytryptophan, 5-methoxypsoralen, 6-mercaptopurine, 6-thioguanine, 9-aminocamptothecin, 9-aminofusin, 9-nitrocamptothecin, abacavir, abafungin, abametapir, abediterol, abexinostat, abiraterone, ABT-072, ABT-751, acadesine, acalabrutinib, acamprosate, acamprosate calcium, acarbose, acebilustat, acebutolol, aceclidine, aceclofenac, aceneuramic acid, acenocoumarol, Acetadote, acetaminophen, acetate-free bicarbonate, acetazolamide, acetic acid, acetylcholine, acetylcysteine, acetyl-L-carnitine, acetyl-L-carnitine hydrochloride, acetyl-L-leucine, acetylsalicylic acid, acetyl-salicylic acid, Acetylsalicylic acid (ASA), acetylsalicylic acid lysinate, aciclovir, acipimox, acitretin, aclarubicin, aclidinium, aclidinium bromide, acolbifene, acorafloxacin, acotiamide hydrochloride, ACP-104, acrivastine, ACT-01, ACT-280778, actinomycin D, acumapimod, acyline, adapalene, ADC-3680, Adderall XR, adefovir dipivoxil, ademetionine, adenosine, adinazolam, adipiplon, adomeglivant, adozelesin, adramycin, adrenalin, adrenaline, adriamycin, Advair, Advil, AE-941, afacifenacin fumarate, afatinib, afegostat, afeletecan, afimoxifene, aflibercept, aftobetin, afuresertib, aganepag isopropyl, agatolimod, agave inulin, agomelatine, Aiphagan, ajmaline, aladorian, alagebrium chloride, alanyl-glutamine dipeptide, albaconazole, albendazole, albiglutide, albitiazolium bromide, albumin, albuterol, albuterol sulphate, albuterpenoids, alcaftadine, alcipotriol/betamethasone, aldesleukin, aldoxorubicin, alectinib, aleglitazar, alemtuzumab, alendronate, alendronate sodium, alendronic acid, Alequel, Aleve, alphacalcidol, alfentanil, alfuzosin, algeldrate/magnesium oxide, Alimta, alisertib, aliskiren, alisporivir, alitretinoin, alizapride, allantoin, allisartan isoproxil, allopregnanolone, allopurinol, all-trans retinoic acid, almorexant, almotriptan, Alodan, alogliptin benzoate, alosetron, alovudine, alpelisib, alpha lipoic acid, alpha tocopherol, alpha-1 antitrypsin, alpha-cyclodextrin, alpha-glucosidase inhibitor, alpha-interferon, alpha-lipoic acid, alpha-tocopherol, alpha-tocopherol acetate, alpha-trichosanthin, alprazolam, alprostadil, alprostadil alphadex, ALS-08, altinicline, Altropane, aluminium MgS, aluminum hydroxide, alvespimycin hydrochloride, alvimopan, alvocidib, amantadine, amantadine hydrochloride, ambrisentan, ambroxol, ambroxol hydrochloride, AMD-070, amdoxovir, amelubant, amenamevir, Ametop, amfetamine, amibegron, amifampridine phosphate, amifostine, amikacin, amiloride, amiloride hydrochloride, amino acid, Aminocaproic Acid, aminoglutethimide, aminoguanidine, aminolevulinic acid, aminolevulinic acid hydrochloride, aminophylline, aminopterin, amiodarone, amiprilose, amiselimod, amisulpride, amitifadine hydrochloride, amitriptyline, Amitriptyline hydrochloride, amlexanox, amlodipine, amlodipine besilate, amlodipine besylate, amlodipine camsylate, amlodipine maleate, ammonium lactate, amnion, amodiaquine, amonafide dihydrochloride, amonafide L-malate, amorolfine, amoxapine, amoxicillin, amoxicillin clavulanate, amoxicillin MR, amoxicillin/clavulanate, amoxicillin-clavulanic acid, amoxycillin, amphetamine, amphetamine aspartate, amphetamine sulphate, amphotericin, amphotericin B, ampicillin, ampicillin sodium, ampicillin/flucloxacillin, amprenavir, amrubicin, amsacrine, amsilarotene, AN-2898, AN-9, anacetrapib, anagliptin, anagrelide, anamorelin, anastrozole, anatibant, ancriviroc, ancrod, androgen, Androxy, anecortave, angiotensin converting enzyme inhibitor, angiotensin I, angiotensin II, Angiozyme, anidulafungin, aniracetam, annamycin, antazoline, anthocyanin, anthracycline, anti-emetic, antihistamine, antilymphocyte globulin, antineoplaston A-10, antineoplaston A10-I, antineoplaston AS2-1, Antioxidant Vitamins, antipsychotic, antiretroviral drugs, antithymocyte globulin, anti-thymocyte globulin, apabet, apadenoson, apaziquone, apelin, apheresis, apilimod, apimostinel, apitolisib, apixaban, aplaviroc, aplindore, apomorphine, Apovir, apratastat, apremilast, aprepitant, apricitabine, apricoxib, aprotinin, AR-623, Ara-C, arachidonic acid, aracytine, Aralast, aramchol, arasertaconazole, arbaclofen, arbaclofen placarbil, arbekacin sulphate, arbutin, ARC-100, arformoterol, argatoroban, argatroban, arginine, arginine vasopressin, ARH-1, arhalofenate, arimoclomol, aripiprazole, armodafinil, arogliptin, arsenic trioxide, artefenomel mesylate, artemether, artemether-lumefantrine combination, artemisinin, artemisone, artemotil, artenimol, arterolane, arterolane maleate, artesunate, artesunate+mefloquine, artesunate-amodiaquine, articaine, articaine hydrochloride, arundic acid, arzoxifene, asapiprant, ASCJ-9, ascorbate, ascorbic acid, asenapine, asimadoline, ASM-024, asoprisnil, aspirin, astaxanthin, astodrimer, asunaprevir, AT-101, ataciguat, atagabalin, ataluren, atamestane, atazanavir, atazanavir sulphate, atazanavilritonavir, atecegatran fexenetil, Atelvia, Atenativ, atenolol, atevirdine, ATHX-105, atiprimod, atiratecan, Ativan, atomoxetine, atopaxar, atorvastatin, atovaquone, atracurium, atracurium besylate, atrasentan, atreleuton, Atripla, atropine, auranofin, auriclosene, AVAC, avacopan, avagacestat, avanafil, avasimibe, avatrombopag, AVE-0657, AVE-2268, avibactam sodium, Avil, avobenzone, avoralstat, avosentan, AWD-12-281, axelopran, Axiron, axitinib, axomadol, azacitidine, azathioprine, AZD-1775, AZD-4547, AZD-9668, Azedra, azelaic acid, azelastine, azelastine hydrochloride, azeliragon, azelnidipine, azidothimidine, azilsartan, azilsartan medoxomil potassium, azimilide, azithromycin, azithromycin dihydrate, azosemide, aztreonam, aztreonam lysine, bacitracin, baclofen, bafetinib, baicalin, balaglitazone, balicatib, balsalazide, bambuterol, banoxantrone, barasertib, bardoxolone methyl, baricitinib, barnidipine, basiliximab, basimglurant, basmisanil, batabulin, batefenterol succinate, bavisant, bazedoxifene, BCG vaccine, BCNU, becatecarin, beclabuvir, beclometasone, beclometasone dipropionate, beclomethasone, beclomethasone dipropionate, becocalcidiol, Beconase, bedaquiline, bedoradrine, bedrocon, belinostat, Belladonna, belnacasan, beloranib, belotecan, bempedoic acid, benazepril, bendamustine, bendroflumethiazide, beneh, benfotiamine, benidipine, benserazide, bentamapimod, benzalkonium, benzalkonium chloride, benzathine penicillin, benzbromarone, benznidazole, benzocaine, benzodiazepine, benzophenone-3, benzoyl peroxide, benztropine, benzydamine hydrochloride, benzylic alcohol, benzylpenicillin, benzylpiperazine, Bepantol, bepotastine, beractant, beraprost sodium, berberine, berubicin, besifloxacin, besifovir, beta erythropoietin, beta-1,3/1,6-glucan, beta-blocker, beta-blockers, beta-carotene, beta-cryptoxanthin, betadine, Betafectin, betahistine, betaine, Betaine anhydrous, beta-lactamase inhibitor, Betamarc, betamethasone, betamethasone dipropionate, betamethasone mousse, betamethasone valerate, betamethasone dipropionate, beta-tricalcium phosphate bone substitute, betaxolol, betaxolol hydrochloride, bethanechol, bethanechol chloride, betrixaban, betulinic acid, bevacizumab, bevenopran, bevirimat, bexagliflozin, bexarotene, bezafibrate, BF-derm1, BGP-15, BI-54903, biapenem, bicalutamide, bicifadine, bifeprunox, Bifidobacterium, Bifidobacterium bifidum, Bifidobacterium infantis 35624, bifonazole, biguanide, BIIB-021, bilastine, BILR-355-BS, bimatoprost, bimoclomol, bimosiamose, bindarit, binimetinib, binodenoson, Bio-25, biotin, biperiden, biphentin, birabresib dihydrate, biricodar, birinapant, bisacodyl, biskalcitrate potassium, bismuth, bismuth citrate, bismuth potassium citrate, bismuth sodium tartrate, bismuth subcitrate, bismuth subsalicylate, bisoprolol, bisoprolol fumarate, bisphosphate, bitopertin, bixalomer, bleomycin, bleomycin sulphate, blonanserin, BMP-7, BNC-105P, boceprevir, boric acid, boron-anticancers, bortezomib, bosentan, bosutinib, bradanicline, bradykinin, Bramitob, branched chain amino acid, brecanavir, brexpiprazole, Bricanyl, Bricasol, brimonidine, brimonidine tartrate, Brinavess, brinzolamide, brivanib alaninate, brivaracetam, brivudine, brolucizumab, bromfenac, bromfenac sodium, bromhexine, bromocriptine, brompheniramine, bronopol, brostallicin, brotizolam, bryostatin-1, BTI-320, BTL-TML-HSV, BTS-67582, bucindolol, budesonide, budesonide/formoterol, budesonine, budiodarone, bumetanide, bunazosin, buparlisib, bupivacaine, bupivacaine hydrochloride, bupivacaine with fentanyl, bupivacaine-clonidine, buprenorphine, buprenorphine hemiadipate hydrochloride, buprenorphine hydrochloride, buprenorphine/naloxone, bupropion, bupropion hydrochloride, bupropion SR, burapitant, burixafor, buserelin, buserelin acetate, buspirone, buspirone hydrochloride, busulfan, Busulfex, butalbital, butenafine, butoconazole, butoconazole nitrate, butorphanol, butorphanol tartrate, C5a, Cabaseril, cabazitaxel, cabergoline, cabotegravir, cabozantinib S-malate, Cacit D3, cadazolid, CAF regimen, caffeic acid, caffeine, caffeine citrate, caffeinol, Calcichew D3 Forte, calcipotriol, calcipotriol/betamethasone, calcitriol, calcium, calcium acetate, calcium ascorbate, calcium carbonate, Calcium chloride, calcium chloride dihydrate, calcium citrate, calcium dobesilate, calcium fluoride, calcium folinate, calcium glucarate, calcium gluconate, calcium hydrogenphosphate, calcium L-aspartate, calcium levofolinate, calcium phosphate, calcium polycarbophil, Calcium sodium phosphosilicate, Calcium supplements, calcium and vitamin D, calcium leucovorin, caldaret, calphactant, camicinal, camobucol, camptothecin, canagliflozin, candesartan, candesartan cilexetil, canertinib, canfosfamide, cangrelor, cannabidiol, Cannabidiol (CBD), cannabidivarin, cantharidin, capadenoson, capecitabine, capmatinib, Capolac, capravirine, Capros, capsaicin, captopril, carbamazepine, carbenoxolone, carbetimer, carbetocin, carbidopa, carbocisteine, carbocysteine, Carbogen, carbon [14C] oxaliplatin, carbon dioxide, carbon monoxide, carbondioxide, carboplatin, Carboxymethylcellulose sodium, cardidopa, cardonutrient, carfilzomib, carglumic acid, cariporide, cariprazine, carisbamate, carisoprodol, carmegliptin, carmoterol, carmustine, carnitine, carotegrast methyl, carteolol, carteolol hydrochloride, carvedilol, carvedilol phosphate, CA5AD, casein, casopitant, caspofungin, catechin, CBT-1, CCPI, cebranopadol, cediranib, cefaclor, cefadroxil, cefalexin, cefazolin, cefazolin sodium, cefdinir, cefditoren pivoxil, cefepime, cefilavancin, cefixime, cefmetazole, cefoperazone, cefotaxime, cefotetan, cefoxitin, cefozopran, cefpirome, cefpodoxime, cefprozil, ceftaroline, ceftaroline fosamil, ceftazidime, ceftibuten, ceftobiprole medocaril, ceftolozane sulphate, ceftriaxone, cefuroxime, celecoxib, Celestone, celgosivir, celiprolol, celivarone, Cenestin, cenicriviroc, cenobamate, censavudine, centanafadine, Centrum forte, Centrum Silver, cephalexin, cephalosporin, ceralifimod, ceramide, ceritinib, cerium nitrate, cerivastatin, cerlapirdine, certoparin, cetaphil, cethromycin, cetilistat, cetirizine, cetylpyridinium chloride, cevimeline, chenodeoxycholic acid, CHF-1535, CHF-5992, chitosan, chitosan-thiomer, chlorambucil, chloramphenicol, chlordiazepoxide, chlorhexidin, chlorhexidine, chlorhexidine digluconate, chlorhexidine gluconate, chlorhexidine-alcohol, chlorhydrate, chlormadinone acetate, chloroprocaine, chloroquine, chloroquine diphosphate, chloroquine phosphate, chlorpheniramine, chlorpheniramine maleate, chlorproguanil, chlorpromazine, chlortalidone, chlorthalidone, chlorzoxazone, cholecalciferol, cholecystokinin-8, cholesterol absorption inhibitors, cholestyramine, cholic acid, choline, choline alfoscerate, choline diepalrestat, choline fenofibrate, choline magnesium trisalicylate, chondroitin, chondroitin sulphate, CHP-HER2, Chromium cobalt, Chromium Picolinate, CHS-131, CHVP-interferon, ciclesonide, cicletanine, ciclopirox, ciclopirox olamine, ciclosporin, ciclosporine A, cidofovir, cilansetron, cilastatin, cilazapril, cilnidipine, cilomilast, cilostazol, ciluprevir, cimetidine, cimicoxib, cinacalcet, cinaciguat, cindunistat hydrochloride maleate, cinhyaluronate sodium, cinitapride, cinitapride tartrate, cinnamaldehyde, cinnarizine, cipargamin, ciprofibrate, ciprofloxacin, ciprofloxacin hydrochloride, ciraparantag, Cisapride, cisatracurium, cisatracurium besilate, cisplatin, cisplatin liposomal, cisplatinum, citalopram, citalopram hydrobromide, Citanest, citicoline, citrate, citrate fentanyl, citric acid, citric acid monohydrate, citrulline, CK-2017357, cladribine, Clarinex, clarithromycin, clavulanate, clavulanate potassium, clavulanic acid, clazosentan, clebopride, clemastine, clemastine fumarate, clenbuterol, clevidipine, clevudine, clindamicin, clindamycin, clindamycin phosphate, clindamycin/benzoyl peroxide, Clinisol, clioquinol, clobazam, clobetasol, clobetasol propionate, Clobex, clodronic acid, clofarabine, clofazimine, clomethiazole edisylate, clomifen, clomifene, clomifene citrate, clomiphene, clomiphene citrate, clomipramine, clonazepam, clonidine, clonidine hydrochloride, clopidogrel, clopidogrel hydrogen sulphate, clopidogrel napadisilate, Cloratadd-D, clorazepate, Clostridium butyricum MIYAIRI 588, clotrimazole, cloxacillin, clozapine, coamoxiclav, cobalamin, cobamamide, cobicistat, cobimetinib, cobiprostone, cocaine, codeine, codeine phosphate, coenzyme Q10, Coeruleus, cogentin, Cogentus, colchicine, colcine, colecalciferol, colecalciferol D3, coleneuramide, colesevelam, colestilan, colestipol, colfosceril palmitate, colistimethate, colistimethate sodium, colistin, colistin sulphate, colistineb, colloidal bismuth, colloidal bismuth tartrate, coloxyl, coluracetam, combretastatin, Comp-01, Comp-02, Comp-03, Comp-04, conivaptan, conivaptan hydrochloride, conjugated estrogen, conjugated estrogens, controlled-release carvedilol, copanlisib, copper, copper histidine, coQ10, cortexolone 17alpha-propionate, corticosteroid, cortisol, cortivazol, cositecan, CosmoFer, cotrifazid, cotrimoxazol, cotrimoxazole, co-trimoxazole, COX-inhibitor, CPI-613, CRAd 3/5-delta, creatine, creatine ethyl ester, creatine monohydrate, crenolanib, crisaborole, crizotinib, CRM-197, crobenetine, crofelemer, cromoglicate, cromoglicic acid, cromolyn sodium, Crsytalloids, C-Tb, CTO, CUDC-305, Curcuma aeruginosa, curcumin, curcuminoids, curdlan sulphate, cutamesine dihydrochloride, CX-516, cyanocobalamin, cyclizine, cyclizine lactate, cyclobenzaprine, cyclobenzaprine hydrochloride, cyclodextrin, cyclodextrin-combined diclofenac, Cyclogest, cyclopentolate, cyclophophamide, cyclophosphamide, cyclophosphamide monohydrate, cyclophosphan, cycloserine, cyclosporin, cyclosporine, cyclosporine A, cyclosporine microemulsion, cyclphosphamide, cyproheptadine, cyproterane acetate, cyproteron, cyproterone, cyproterone acetate, cysteamine, Cysteamine hydrochloride, cysteine, cysteine hydrochloride monohydrate, cytarabine, cytarabine arabinoside, cytarabine-asparagine conjugate, cytophosphan, Cytosin-Arabinosid, cytosine arabinoside, cytoxan, Cytozar, D3 vitamine, DA-9601, dabigatran etexilate, dabrafenib, dacarbazine, daclatasvir, daclizumab, dacomitinib, dactinomycin, dactolisib, daglutril, daidzein, dalbavancin, dalcetrapib, dalfopristin, dalteparin sodium, D-amphetamine, danazol, danirixin, danoprevir, dantrolene, dantrolene sodium, danusertib, dapaconazole, dapagliflozin, dapagliflozin propanediol, dapansutrile, dapivirine, daporinad, dapoxetine, dapsone, dapsone gel, darapladib, darifenacin, darinaparsin, darolutamide, darotropium bromide, darunavir, darunavir/ritonavir, darusentan, dasabuvir, dasatinib, dasotraline, daunorubicin, daunorubicin hydrochloride, daunorubicine, D-cycloserine, dDAVP, DDP, DE-104, DE-110, DE-112, Deanxit, Debio-1450, Debio-1452, decadron, decarbazine, decernotinib, decitabine, decoglurant, Decuprate, defactinib, deferasirox, deferiprone, deferitazole, deferoxamine, deferoxamine mesylate, deflazacort, dehydroepiandrosterone, delafloxacin, delamanid, delanzomib, delapril, delapril hydrochloride, delavirdine, Delazine, deleobuvir, deligoparin sodium, delorazepam, delta-8-THC, delta-9-tetrahydrocannabinol, denagliptin, denufosol tetrasodium, Depacon, Depade, depo-medroxyprogesterone, depomedroxyprogesterone acetate, depomethylprednisolone, depotestosterone, DER-45-EV, derenofylline, dersalazine sodium, desferrioxamine, desflurane, desipramine, desloratadine, desmopressin, desmopressin acetate, desogestrel, desonide, desote fumarate, desoximetasone, desvenlafaxine, Detox-B adjuvant, deutetrabenazine, dexamethasone, dexamethasone acetate, dexamethasone cipecilate, dexamethasone diphosphate, dexamethasone phosphate, dexamethasone sodium phosphate, dexamfetamine, dexanabinol, dexchlorpheniramine, dexedrine, dexelvucitabine, dexfenfluramine, dexibuprofen, Dexid, dexisometheptene mucate, dexketoprofen, dexketoprofen trometamol, dexlansoprazole, dexlipotam, dexloxiglumide, dexmecamylamine, dexmedetomidine, dexmethylphenidate, dexniguldipine, dexpanthenol, dexpramipexole, dexrazoxane, dexrazoxane hydrochloride, dextofisopam, dextran, dextroamphetamine, dextroamphetamine saccharate, dextroamphetamine sulphate, dextromethorphan, dextromethorphan hydrobromide, dextropropoxyphene, dextropropoxyphene hydrochloride, dextrose, dexverapamil, dezocine, DHEA, diacerein, diacetylmorphine, Dialysate calcium, Diamel, diammindichloridoplatin, diamorphine, diamorphine hydrochloride, dianhydrogalactitol, dianicline, Diao Xin Xue Kang, diazemuls, diazepam, diazepam autoinjector, diazoxide, diazoxide choline, dibasic dihydrate sodium phosphate, dibasic sodium phosphate, dibekacin, dichlorphenamide, Diclazuril, diclofenac, diclofenac diethylamine, diclofenac potassium, diclofenac sodium, dicloxacillin, didanosine, dienogest, diethylcarbamazine, diethylnorspermine, diethylpropion, diethylstilbestrol, diflomotecan, diflunisal, difluprednate, digitoxin, digoxin, dihematoporphyrin, dihomo gamma-linolenic acid, dihydralazine, dihydroartemisinin, dihydroartemisinin-piperaquine, dihydrocodeine, dihydroergotamine, dihydroergotamine mesylate, di hydroxy vitamin D3, diiodothyropropionic acid and its analogs, diiphenhydramine, dilaudid, dilmapimod, diltiazem, diltiazem hydrochloride, dimenhidrinate, dimenhydrinate, dimesna, dimethindene, dimethindene maleate, dimethyl fumarate, dimethylfumarate, dimiracetam, dinoprostone, diosmin, diphencyprone, diphenhydramine, diphenylcyclopropenone, dipirone, dipraglurant-IR, dipyradimole, dipyridamole, dipyrone, diquafosol sodium, diquafosol tetrasodium, disopyramide, Dispase II, disufenton sodium, disulfiram, dithranol, ditiocarb sodium, DLBS-1033, DLBS-1425, D-methadone, DNE3, dobutamine, docetaxel, dociparstat, doconexent, doconexent ethyl ester, docosahexaenoic acid [DHA], docosahexaenoic acid monoglycerides, docosanol, docusate, docusate sodium, dofetilide, dolasetron, dolastatin-10, dologesic, dolutegravir, domperidone, donepezil, donepezil hydrochloride, donu, dopamine, dopexamine, doramapimod, doravirine, doripenem, dorzolamide, dorzolamide hydrochloride, dorzolamide hydrochloride+timolol maleate, dothiepin, dovitinib, doxapram, doxazosin, doxazosin mesylate, doxepin, doxepin hydrochloride, doxercalciferol, doxifluridine, Doxil, doxophylline, doxorubicin, doxorubicin etarfolatide, doxorubicin HCl liposome, doxorubicin hydrochloride, doxorubicin hydrochloride liposome, doxycycline, doxycycline hyclate, doxylamine, doxylamine succinate, D-penicillamine, DPP-IV inhibitors, DPS-102, draflazine, drinabant, dronabinol, dronedarone, droperidol, dropropizine, drospirenone, drotaverine, droxidopa, D-tagatose, D-TRANS fentanyl, Duac, dual-release hydrocortisone, dulaglutide, Dulcolax, duloxetine, Duracain, duramorph, Durolane, dutasteride, dutogliptin, duvelisib, duvoglustat, D-xylose, dydrogesterone, dyhydroprogesterone, DZ-1, E coli Nissle, E-7016, E-7820, ebastine, ebselen, EC-17, ecabet, ecabet sodium, Echinacea, econazole nitrate, ecopipam, ecosprin, ecraprost, Ecural, edaglitazone, edaravone, edetate calcium disodium, edivoxetine, edonerpic maleate, edotecarin, edoxaban, EES0000645/A, efaproxiral, efatutazone, efavirenz, efinaconazole, eflornithine, efonidipine hydrochloride, EGb-761, EGCG, eicosapentaenoic acid, eicosapentanoeic acid, elacestrant, elacridar, elacytarabine, elafibranor, elagolix, elamipretide, elbasvir, elbion, eldecalcitol, eleclazine, elesclomol sodium, eletriptan, eliglustat tartrate, elinogrel, eliprodil, ELND-005, elobixibat, elocalcitol, Elomet, Elosalic, elsamitrucin, eltoprazine, eltrombopag, elubrixin, eluxadoline dihydrochloride, elvitegravir, elvorin, elvucitabine, elzasonan, Emdogain, emedastine, emepepimut-S, emicerfont, emivirine, emixustat, empagliflozin, emricasan, emtricitabine, enalapril, enalapril maleate, enalaprilat, enasidenib, encaleret sulphate, enclomifene citrate, enclomiphene, encorafenib, endocannabinoid palmitoylethanolamide, endonase, endotoxin, endoxan, enecadin, enflurane, enfuvirtide, eniluracil, ENMD-2076, enobosarm, enocitabine, enoxaparin sodium, enoximone, entacapone, entecavir, entecavir maleate, enteric-coated mycophenolate sodium, enteric-coated tegafur-uracil, Enteroaggregative E coli, entinostat, entonox, enzalutamide, enzastaurin, epacadostat, Epadel, epalrestat, eperisone, eperisone hydrochloride, epetirimod, epetraborole, ephedrine, Epiceram, epidoxirubicin, epidoxorubicin, epidural/paravertebral analgesia, epigallocatechin gallate, epigallocatechin-3-gallate, epigallocatechin-gallate, Epiggallocatechin, epinastine, epinastine hydrochloride, epinephrine, epirubicin, epirubicin hydrochloride, Episalvan, eplerenone, eplivanserin, epoprostenol, Eppikajutsutou, eprodisate, eprosartan, eprotirome, epsilon-aminocaproic acid, eptastigmine, eptifibatide, Equine antithymocyte immunoglobulin, Equisetum arvense, eravacycline, erdosteine, Eremostachys laciniata, ergocalciferol, ergotamine, eribaxaban, eribulin mesylate, Eritex, eritoran, erlotinib, ertapenem, erteberel, ertugliflozin, erythromycin, erythromycin lactobionate, erythropoetin, erythropoietin beta, ESAT-6CFP10, esaxerenone, Escherichia coli endotoxin, escitalopram, esflurbiprofen, Eskalith, esketamine, esketamine hydrochloride, eslicarbazepine acetate, Esmeron, esmirtazapine, esmolol, esmolol hydrochloride, esomeprazole, esoxybutynin, esreboxetine, estazolam, estetrol, estradiol, estradiol acetate, estradiol cypionate, estradiol valerate, estradiol/norethindrone acetate, estramustine, estramustine phosphate, estramustine phosphate sodium, Estratest, estriol, estriol E3, estrodiol, estrogen, estrogens, eszopiclone, etacrynic acid, etalocib, Etalpha, etanercept biosimilar, etazolate, Ethacrynic acid, ethambutol, ethambutol hydrochloride, ethanol, ethinyl estradiol, ethinyl estradiol/levonorgestrel, ethinylestradiol, ethiodized oil, ethosuximide, ethyl chloride, ethyl eicosapentaenoate, ethyl hydrogen fumarate calcium, ethyl hydrogen fumarate magnesium, ethyl hydrogen fumarate zinc, ethylenediaminetetraacetate, ethylhexyl triazone, ethynylcytidine, etidronic acid, etilefrine, etodolac, etomidate, etomidate Lipuro, etomoxir, etonogestrel, etonox, etoposide, etoposide phosphate, etoricoxib, etravirine, EV-06, evacetrapib, evatanepag, everolimus, eviprostat, evofosfamide, evogliptin, exatecan, exemestane, exenatide, exeporfinium chloride, exisulind, ezatiostat, ezetimibe, ezetimibe/atorvastatin, F-0434, F0-M0, F0-M1700, F160-M0, F160-M1000, F160-M1700, F80-M1000, F80-M1700, FA, facinicline hydrochloride, faldaprevir, famciclovir, famitinib L-malate, famotidine, fampridine, Fangji, farampator, farglitazar, faropenem, faropenem medoxomil, fasiglifam hemihydrate, fasitibant chloride, fasudil, favipiravir, FBG-18, FBP peptides, FE[50]C, FE[75]C, Fe-58, febuxostat, fedovapagon, fedratinib, felbamate, felbinac, felodipine, fenatnyl, fenobam, fenofibrate, fenoldopam, fenoterol, fenoterol prednisone, fenretinide, fentanyl, fentanyl citrate, fermagate, ferric carboxymaltose, ferric citrate, ferric maltol, ferric pyrophosphate, Ferripel-3, ferroquine, ferrous fumarate, ferrous sulphate, ferumoxtran-10, ferumoxytol, FeSO4, fesoterodine fumarate, fevipiprant, fexinidazole, fexofenadine, fiboflapon, fibrinogen, fidaxomicin, filanesib, filgotinib, filgrastim, filibuvir, filorexant, fimaporfin, fimasartan, finafloxacin, finafloxacin hydrochloride, finasteride, finerenone, fingolimod, Fioricet, fipamezole, fish oil (eicosapentaenoic acid [EPA] plus docosahexaenoic acid [DHA]), Fisiogel, fispemifene, Flagyl, Flavan-3-ol, flavanone, flavoxate, flecainide, flibanserin, flomoxef, flomoxef sodium, flopristin, florbenazine, florbetapir, flortaucipir F 18, flourinic acid, flovagatran, floxuridine, fluciclatide F 18, flucloxacillin, fluconazole, flucytosine, fludarabine, fludeoxyglucose, fludeoxyglucose F 18, fludrocortisone, flumazenil, flunarizine, flunisolide, fluocinolone acetonide, fluocinonide, fluorescein, fluorometholone, fluorometholone acetate, fluoropyrimidine, fluoroquinolones, fluorouracil, fluoxetine, fluoxetine hydrochloride, flupenthixol, flupentixol, fluphenazine, flupirtine, flurbiprofen, flurbiprofen axetil, flurbiprofen sodium, flutamide, fluticasone, fluticasone furoate, fluticasone propionate, flutrimazole, fluvastatin, fluvoxamine, FM-VP4, folacin, folate, folate/iron, FOLFIRI, FOLFOX4, FOLFOXIRI, folic acid, folinate, folinic acid, folitixorin calcium, follitropin beta, fonadelpar, fondaparinux sodium, Foradil, foretinib, formoterol, formoterol fumarate, forodesine, foropafant, fosalvudine tidoxil, fosamprenavir, fosamprenavir calcium, fosaprepitant, fosbretabulin, fosbretabulin disodium, foscarnet, foscarnet sodium, fosdagrocorat, fosdevirine, fosfluridine tidoxil, fosfomycin, fosfomycin trometamol, fosfructose, fosinopril, fosmidomycin, fosphenytoin, fospropofol, fostamatinib, fostemsavir tromethamine, Fostimone, Fostrap, fotemustine, fozivudine tidoxil, freselestat, Fresubin, frovatriptan, fructose, fructose-1,6-diphosphate, fruquintinib, frusemide, fucoidan, fulvestrant, fumarate, funapide, furaprevir, furazolidone, furosemide, fusidate sodium, fusidic acid, Fuzheng Huayu, gabapentin, gabapentin enacarbil, gaboxadol, gadobenic acid, gadobutrol, gadofosveset, gadolinium, gadopentetate dimeglumine, gadoterate meglumine, gadoversetamide, gadoxetate disodium, Galactooligosaccharide, Galacto-oligosaccharides, galantamine, galantamine CR, galeterone, gallium maltolate, gallium nitrate, gallopamil, gambogic acid, Gamma-Linolenic Acid, gamma-tocopherol, ganaxolone, ganciclovir, ganciclovir phosphonate, ganetespib, ganirelix acetate, ganstigmine, garenoxacin, garlic, gatifloxacin, GCS-100, G-CSF, gedatolisib, gefitinib, gelatin, Gelofusine, Gelpart, gemcabene, gemcitabine, gemcitabine elaidate, gemcitabine prodrug, gemfibrozil, gemifloxacin, gemigliptin, gemigliptin tartaric acid, Gemzar, Genaera, genistein, genistein+decitabine, gentamicin, gentamicin sulphate, gentamycin, gepirone, gepotidacin, gestodene, gestodone, gestrinone, gilteritinib, gimatecan, gimeracil, Ginkgo biloba, ginkgolides meglumine, ginsenoside Rg3, ginsenoside-Rd, giripladib, gisadenafil, givinostat, GKT-831, glatiramer acetate, glecaprevir, glesatinib glycolate, glibenclamide, gliclazide, glimepiride, glinide, glipizide, glitazone, GLP-1 analog, glucagon-like peptide-1, glucocorticoids, glucocorticosteroid, glucosamine, glucosamine hydrochloride, glucose, glufosfamide, glutamic acid, glutamine, glutathione, glycerin, glycerol, glycerol phenylbutyrate, glycoprotein IIb/IIIa inhibitor, glycopyrolate, glycopyrrolate, glycopyrronium bromide, glycopyrronium tosylate, glycopyrroniumbromide, glycyrrhizin, glyminox, GM1, GM-CT-01, GnRH antagonist, gold sodium thiosulphate, golotimod, golvatinib tartrate, gonadotopin, gonadotropin, gonadotropins, GoodBelly probiotic, goserelin acetate, goshajinkigan, gosogliptin, gp100, GPO-Vir Z30, granisetron, granotapide, grazoprevir, grepafloxacin, griseofulvin, GR-MD-02, GSK-2269557, GSK-2330672, GSK-2339345, guaifenesin, guanethidine, guanfacine, Guizhi fulings, gusperimus trihydrochloride, GWP-42004, gycerol, Gynostemma pentapyllum, H2 blockers, haldol, halofuginone, haloperidol, haloperidol decanoate, halothane, Hangeshashin-to, HBW, HE-3286, Healon, Helico DR, heliox, heme arginate, hemin, hemoximer, heparin, heparin sodium, Her-2/neu, heroin, hexachlorophene, hexaminolevulinate hydrochloride, Hextend, HF-0220, Hibiscus sabdariffa, hidrotalcid, himantane, histamine dihydrochloride, homatropine, honey, hpFSH, HPP-404, HQK-1004, huachansu, Huai Qi Huang, human chorionic gonadotrophin, huperzine A, Hyabest J, hyaluronan, hyaluronate sodium, hyaluronic acid, hyaluronic acid hydrogel, hydralazine, hydrochloric acid, hydrochlorothiazide, hydrochlorothiazide tablet, hydrochlorthiazide, hydrocodone, hydrocodone bitartrate, hydrocodone/acetaminophen, hydrocortisone, hydrocortisone sodium succinate, hydrocortisone-17-butyrate, hydrocodone, hydrogel, hydrogen peroxide, hydromorphine, hydromorphone, hydromorphone hydrochloride, hydroquinidine, hydroquinone, hydroxocobalamin, hydroxycarbamide, hydroxychloroquine, hydroxydaunorubicin, hydroxyethyl starch, hydroxyethylstarch solution, Hydroxyl-propyl-methyl cellulose powder, hydroxymethylbutyrate, hydroxynortriptyline, hydroxyprogesterone caproate, hydroxypropyl cellulose, hydroxytryptophan, hydroxyurea, hydroxyzine, hylastan, Hylenex recombinant, hyoscine butylbromide, hyoscine hydrobromide, hyoscine N-butylbromide, hyoscyamine sulphate, hyperbaric bupivacaine, hypericin, Hypericum perforatum, hyperosmolar dextrose, hypertonic saline, hypromellose, ibandronate, ibandronic acid, iberogast, iberogast N, IBH-B, ibipinabant, ibodutant, ibopamine, ibrutinib, ibudilast, ibuprofen, ibutamoren mesylate, ibuterol, ibutilide, icaritin, icodextrin, icofungipen, icosabutate, icosapent, icosapent ethyl, icosapent ethyl ester, icotinib hydrochloride, idalopirdine, idarubicin, idazoxan, IdB-1016, idebenone, idelalisib, idoxuridine, idrabiotaparinux sodium, idraparinux sodium, idronoxil, iferanserin, ifetroban, ifetroban sodium, IFN-alpha2b, Ifosfamide, iguratimod, IHBG-10, IL-2, ilaprazole, ilepatril, iloperidone, iloprost, iloprost betadex clathrate, imagabalin, imatinib, ImCOOH, imeglimin, imexon, imidafenacin, imidapril, imiglitazar, imipenem, imipramine, imiquimod, imisopasem manganese, IMO-2125, implitapide, incyclinide, indacaterol, indacaterol acetate, indacaterol maleate, indacaterol xinafoate, indantadol, indapamide, indapamide SR, indeglitazar, indinavir, Indinol Forto, indiplon, indisetron, indisulam, Indium In 111 anti-CD66 monoclonal antibody BW250/183, indocyanine green, indometacin, indomethacin, indoramin, industrial nitric oxide, inecalcitol, INF-alpha, infigratinib, infliximab, lngavirin, ingenol mebutate, inhaled sodium nitrite, iniparib, injectable progestin, inosine, inosine pranobex, inositol, INS-1, insulin, insulin glargine, insulin NPH, intepirdine, interferon, interferon alpha, interferon alpha-2a, interferon alpha-2b, interferon beta-1a, interferon beta1b, Interleukin, interleukin-2, interleukin-6, intetumumab, Intracel, intranasal ketamine, intravenous immunoglobulin, intravenous minocycline, iobenguane I-131, iodine, iodine I 131 ethiodized oil, iodine I 131 monoclonal antibody BC8, iodine tincture, iodixanol, iohexol, iopamidol, iopromide, ipragliflozin, ipratropium, ipratropium bromide, IPX-159, IPX-231, irbesartan, irinotecan, irinotecan hydrochloride, irinotecan sucrosofate, irofulven, iron, iron folic acid, iron hydroxide polymaltose, iron oxide, iron proteinsuccinylate, iron solution, iron sucrose, iron supplements, irosustat, irsogladine maleate, IRX-5183, ISA-51, isavuconazonium chloride/sulphate, Iscar, iseganan, isobide, isocaloric diet, isocarboxazid, isoflavone, isoflavones, isoflurane, isolyte-S, isoniazid, isoniazide, isoprinosine, isopropyl alcohol, isopropyl unoprostone, isoproterenol, isoquercetin, isosorbide dinitrate, isosorbide mononitrate, isosorbide-5-mononitrate, isosulfan blue, isotretinoin, isovaleramide, ispinesib, ispronicline, Isradipine, israpafant, istaroxime, itacitinib, itasetron, itopride hydrochloride, itraconazole, itriglumide, ivabradine, ivabradine hydrochloride, ivacaftor, ivermectin, ixabepilone, ixazomib citrate, Jin Fu Kang, JNJ-56914845, Jobelyn, josamycin, Juglans regia extract, Juvidex, Kamikihi-to, kanamycin, kava, KD-018, Keflin, Kenalog, Kenalog-10, ketamine, ketamine hydrochloride, ketanserin, ketoconazole, ketoprofen, ketorolac, ketorolac tromethamine, ketotifen, KLH, Krestin, KRX-0402, KT6-971, KW-2450, KW-2478, KWA-0711, KX2-391, L9NC, labetalol, labradimil, lacidipine, Lacidofil Strong, lacosamide, lactated ringer's solution, lactic acid, LACTIN-V, lactitol, lactobacillus, Lactobacillus acidophilus, Lactobacillus acidophilus KS400, Lactobacillus casei rhamnosus, Lactobacillus delbrueckii, Lactobacillus paracasei F19, Lactobacillus paracasei LP-33, Lactobacillus plantarum 299v, Lactobacillus reuteri, Lactobacillus rhamnosus, Lactobacillus Sporogens, lactose, lactose monohydrate, lactulose, ladarixin, ladostigil, lafutidine, L-alanosine, lamivudine, lamotrigine, landiolol, lanicemine, laninamivir octanoate, laniquidar, lanoconazole, lanopepden, lanperisone, lansoprazole, lanthanum carbonate, lapaquistat, lapatinib, laquinimod, L-arginine, laromustine, laropiprant, larotaxel, L-ascorbic acid, lasmiditan, lasofoxifene, L-asparaginase, latanoprost, latanoprost, latanoprostene bunod, latrepirdine, lauric acid, lazabemide, LC-150444, L-carnitine, L-citrulline, LCL-161, Lcr-35, L-dopa, lecovorin, lecozotan, lecozotan SR, lederfolin, ledipasvir, lefamulin, leflunomide, lemborexant, lemuteporfin, lenalidomide, lenograstim, lentinan, lentinan viral, lenvatinib mesylate, LEO-80122, L-epinephrine, lercanidipine, lersivirine, lesinurad, lesogaberan, lestaurtinib, letaxaban, leteprinim, letermovir, letrozole, leucine, leucoverin, leucovorin, leucovorin calcium, leukapheresis, leukotriene B4 (LTB4), leuprorelide, leuprorelin acetate, levalbuterol, levalbuterol hydrochloride, levamfetamine, levamisole, levamlodipine, levamlodipine besylate, levetiracetam, levobetaxolol, levobupivacaine, levocabastine hydrochloride, levocarnitine, levocetirizine, levocetirizine di hydrochloride, levodopa, levofloxacin, levofolinate, levogestrel, levoketoconazole, levoleucovorin, levo-leucovorin, levomequitazine, levomilnacipran, levonorgestrel, levo-phencynonate hydrochloride, levorphanol, levosalbutamol, levosimendan, levosulpiride, levothyroxine, levothyroxine sodium, levotofisopam, lexibulin, lexipafant, L-folinic acid, L-glutamine, LH-RH agonist, liafensine, liarozole, Libifem, licarbazepine, licochalcone A, licofelone, lidocaine, lidocaine chlorhydrate, lidocaine-prilocaine, lifibrol, lifitegrast, lignocaine, LIK-066, limaprost, Limtop, linagliptin, linaprazan, lincomycin, linezolid, linifanib, linoleic acid, linopristin, linsitinib, liothyronine, liothyronine sodium, lipid, lipiodol, Lipiodol-ethanol mixture, Lipocine, LipoCol, lipoic acid, lipopolysaccharide, liposomal amphotericin B, liposomal cisplatin, liposomal doxorubicin, liposomal paclitaxel, liposomal prostaglandin E-1, liposomal vincristine, Liproca Depot, lisavanbulin hydrochloride, lisdexamfetamine, lisinopril, lisofylline, lisuride, lithium, lithium carbonate, Lithium citrate, lithium salt, litronesib, lixivaptan, L-leucovorin, L-leucyl-L-leucine methyl ester, L-NMMA, lobaplatin, lobeglitazone, lobeline, lobeline sulphate, lobucavir, lodenafil carbonate, lodenosine, lofepramine, lofexidine, lomibuvir, lomitapide, lomustine, Lomustine (CCNU), lonafarnib, lonaprisan, Long chain fatty acids, long-chain polyunsaturated fatty acids, lonidamine, loperamide, loperamide hydrochloride, loperamide oxide, lopinavir, lopinavir/ritonavir, loratadine, lorazepam, lorcaserin, lorediplon, lormetazepam, L-ornithine L-aspartate, lornoxicam, losartan, losartan potassium, losigamone, losmapimod, loteprednol etabonate, lovastatin, loxapine, loxoprofen, lubiprostone, lucanthone, lucitanib hydrochloride, luliconazole, lumacaftor, lumateperone toluenesulfonate, lumefantrine, lumicitabine, luminespib, lumiracoxib, lunacalcipol, lurasidone, lurbinectedin, Lurotin, lurtotecan, luseogliflozin hydrate, lusutrombopag, lutein, LY-2090314, LY-2623091, Lybrido, lycopene, lymecycline, lynestrenol, Lysomucil, macimorelin, macitentan, Macrolids, mafenide, mafosfamide, MAGE-A1, MAG-EPA oil, magnesium, magnesium aluminum hydroxide, magnesium carbonate, magnesium chloride, magnesium chloride hexahydrate, magnesium citrate, magnesium gluconate, magnesium hydroxide, magnesium isoglycyrrhizinate, magnesium oxide, magnesium sulphate, magnesium valproate, malathion, managlinat dialanetil, mangafodipir, manganese, manidipine, manidipine dihydrochloride, manitimus, mannitol, mapracorat, maprotiline, maralixibat chloride, maraviroc, maribavir, Marijuana, marimastat, marvelone, masilukast, masitinib, masoprocol, mavoglurant, maxacalcitol, mazindol, MCC-135, MDR1, MDT-10013, mebendazole, mebeverine, mebeverine hydrochloride, mecamylamine, mechlorethamine, meclinertant, meclizine, mecobalamin, mecobalamin monohydrate, Medium chain fatty acids, medroxyprogesterone, medroxyprogesterone acetate, mefenamic acid, mefloquine, megestrol, megestrol acetate, meglumine antimoniate, melagatran, Melan-A, melarsoprol, melatonin, meldonium, melfalan, meloxicam, melperone, melphalan, melphalan hydrochloride, memantine hydrochloride, menaquinone, menaquinone-7, menatetrenone, Meniace, menotropin, menotropins, menstrogol, mepacrine, meperidine, mephalan, mepivacaine, mepivacaine chlorhydrate, mepivacaine hydrochloride, mepridine, MER-104, merbarone, mercaptamine, mercaptamine bitartrate, mercaptopurine, mericitabine, merimepodib, meropenem, mesalamine, mesalazine, mesna, metadoxine, Metafolin, Metaglip, metamizole, metamizole sodium, metaraminol, Meteospasmyl, metformin, metformin glycinate, metformin HCl, metformin hydrochloride, metformin SR, methacholine, methadone, methadone hydrochloride, methamphetamine, methazolamide, methimazole, methionine, methocarbamol, methohexital, methotrexate, methotrimeprazine, methoxsalen, methoxyflurane, methoxypsoralen, methyl aminolevulinate hydrochloride, methyl prednisolone, methyl prednisolone acetate, methylcobalamin, methyldibromoglutaronitrile, methyldopa, methylene blue, methylnaltrexone bromide, methylphenidate, methylphenidate hydrochloride, methylprednisolone, methylprednisolone aceponate, methylprednisolone acetate, methylprednisolone sodium succinate, methylprednisone, methylsamidorphan, methylsulfonylmethane, methyltetrahydrofolate, methylthioninium chloride, metirosine, Metobes-compound, metocloperamide, metoclopramide, metolazone, metoprolol, metoprolol succinate, metoprolol tartrate, metoprolol XL, metranidazole, MetroGel, metronidazole, metronomic cyclophosphamide, metyrapone, mexiletine, mexiletine hydrochloride, Mexoryl SX, Mexoryl XI+titanium dioxide, mianserin, mibampator, MIBG, miconazole, miconazole nitrate, microalgal oil, micronized progesterone, micronutrient mixture, midazolam, midazolam hydrochloride, middle-chain and polyunsaturated fatty acids, midodrine, midostaurin, mifepristone, miflonide, migalastat, miglitol, miglustat, milataxel, milnacipran, milrinone, miltefosine, milveterol, mimopezil, minocycline, minocycline hydrochloride, minodronic acid, minoxidil, mirabegron, miriplatin hydrate, mirodenafil, mirtazapine, misoprostol, mitemcinal, mitiglinide, mitoguazone, mitolactol, mitomycin, Mitomycin C, mitoquinone/mitoquinol redox mixture, mitotane, mitoxantrone, mivacurium, mivacurium chloride, mivobulin, mixed salt amphetamine, mizolastine, mizoribine, MK-0782, MK-0893, MK-2206, MK-7622, MK-8457, MMF, mocetinostat dihydrobromide, moclobemide, modafinil, moexipril, molidustat, molindone, molsidomine, molybdenum, momelotinib, mometasone, mometasone furoate, monolaurin, monosodium glutamate, montanide ISA-51, montelukast, montelukast sodium, moracizine, morphine, morphine chloride, morphine glucuronide, morphine hydrochloride, morphine sulphate, mosapride, motesanib diphosphate, motexafin gadolinium, motexafin lutetium, motolimod, moxidectin, moxifloxacin, moxifloxacin hydrochloride, moxonidine, MP-435, MSC-apceth-111, MT-102, Mucaine, Mucopolysaccharide, Mucuna pruriens, multivitamins, muparfostat sodium, mupirocin, muraglitazar, mustine, Mycobacterium w, mycophenolate acid, mycophenolate mofetil, mycophenolate sodium, mycophenolic acid, mycostatin, Mydriasert, Myfenax, Myrtus communis L, mytomycin, N20-02, nabilone, nabiximols, nabumetone, N-acetyl cysteine, N-acetylcisteine, N-acetylcysteine, N-acetyl-L-cysteine, N-acetyl-p-aminophenol, NaCl, nadifloxacin, nadolol, nadroparin calcium, nafamostat, nafamostat mesilate, nafarelin, NaFeEDTA, naftifine, naftifine hydrochloride, naftopidil, nalbuphine, nalbuphine sebacate, naldemedine, nalfurafine, nalmefene, naloxegol, naloxone, naltrexone, naltrexone hydrochloride, naltrexone-poly(DL-lactide), naluzotan, Namodenoson, nandrolone, Naoxintong, napabucasin, naphthoquine, naproxcinod, naproxen, naproxen etemesil, naproxen sodium, naratriptan, naronapride, narrowband UVB, nasapaque, nastorazepide calcium, natamycin, nateglinide, navamepent, navarixin, naveglitazar, navitoclax, N-chlorotaurine, nebentan, nebicapone, nebivolol, nebulized amikacin, nebulized budesonide, Nebusal, nedaplatin, nefazodone, nefiracetam, neflamapimod, nefopam, neladenoson bialanate, nelarabine, nelfinavir, nelivaptan, nelociguat, nelonicline, nelotanserin, nemonoxacin, nemorubicin, neomycin, neomycin sulphate, neostigmine, nepadutant, nepafenac, nepicastat, neramexane, neratinib, neridronic acid, nerispirdine, netarsudil, netilmicin, netivudine, netupitant, Neu-120, neurotropin, nevirapine, niacin, niacinamide, Niacor, NIC5-15, nicardipine, nicergoline, Niclosamide, niconinamide, nicorandil, nicotinamide, nicotine, nicotine polacrilex, nicotinic acid, nifedipine, nifurtimox, Nigella sativa, nikkomycin Z, nilotinib, nilutamide, nilvadipine, nimesulide, nimodipine, nimorazole, nimotuzumab, nimustine hydrochloride, nintedanib, nipradilol, niraparib, nirogacestat, nitazoxanide, nitisinone, nitrazepam, nitrendipine, nitric oxide, nitroflurbiprofen, nitrofurantoin, nitrogen gas, nitroglycerin, nitroglycerine, nitroprusside, nitrous oxide, Nitrousoxide, nivocasan, nizatidine, N-Lite, N-monomethyl-L-arginine, Nobactine, nolatrexed, nolpitantium besilate, nomegestrol acetate, non-amlodipine calcium channel blockers, nonoxynol-9, non-preserved latanoprost, norelgestromin, norepinephrine, norethandrolone, norethindrone, norethindrone acetate, norethisterone, norethisterone acetate, norethisterone enantate, norethrindone acetate, norfloxacin, norgestimate, nortriptylatine, nortriptyline, norursodeoxycholic acid, noscapine, novabupivacaine, Novasoy, Novotaks, NPC-18, NRTI, NS-2, NS-8, NTC-510A, NucleomaxX, nucleoside antiretroviral drugs, NVB, nystatin, Nystatin LF, Nyxol, O2, obatoclax, obeticholic acid, obicetrapib, obinepitide, OBT, ocaperidone, ocinaplon, octinoxate, octocrylene, octocrylene+tinosorb, octreotide acetate, odalasvir, odanacatib, odiparcil, Odyliresin, oestrogen, ofloxacin, oglemilast, oglufanide disodium, olanzapine, olanzapine/fluoxetine, olaparib, olcegepant, oleic acid, olepra, olesoxime, oliceridine, OligoG CF-5/20, olmesartan, olmesartan cilexetil, olmesartan medoxomil, olodaterol, olodaterol hydrochloride, olopatadine, olopatadine hydrochloride, olprinone, olsalazine, oltipraz, omacetaxine mepesuccinate, omadacycline, omapatrilat, omarigliptin, omaveloxolone, ombitasvir, ombrabulin, omecamtiv mecarbil, omega, omega-3, omega-3 carboxylic acids, omega-3 fatty acids, Omega-3 polyunsaturated fatty acids, omega-3-acid ethyl esters, omega-3-carboxylic acids, omega-6, OmegaMAX, Omegaven, omeprazole, omeprazole and bicarbonate, omeprazole sodium, o-methylphenidate, omidenepag isopropyl, omigapil, Ommaya reservoir, Omri-Hep-B, onalespib, oncovin, ondansetron, ondelopran, opicapone, opipramol, opium, fumagillin, orantinib, orbofiban, Org-9426, orilotimod, oritavancin, orlistat, ornithine phenylacetate, orphenadrine citrate, ortataxel, orteronel, Orthokine, orthosilicic acid, Orthostat, Orthostat-L, Orthovisc, orvepitant, oseltamivir, osemozotan, OSI-632, osilodrostat, ospemifene, OsteoDex, Osteonil, otenabant, oteracil potassium, oteseconazole, otilonium bromide, oxacillin, oxaliplatin, oxandrolone, oxantel pamoate, oxazepam, oxcarbazepine, oxidized glutathione sodium, Oximax, oxitriptan, oxitropium bromide, OX-NLA, oxybuprocaine, oxybutinin, oxybutynin, oxybutynin chloride, oxybutynin hydrochloride, oxycodone, oxycodone CR, oxycodone extended-release, oxycodone hydrochloride, oxycodone IR, Oxycyte, oxygen, oxymetazoline, oxymetazoline hydrochloride, oxymetholone, oxymorphone ER, oxymorphone IR, oxypurinol, oxytocin, ozagrel, ozagrel hydrochloride, ozanimod, ozenoxacin, P-276-00, P-53, PAC-14028, paclitaxel, paclitaxel poliglumex, paclitaxel-PM, pacritinib, pactimibe, pafuramidine, pagoclone, palanosetron hydrochloride, palbociclib, palifosfamide, paliperidone, paliperidone ER, paliperidone palmitate, paliroden, palivizumab, palonosetron, palovarotene, pamapimod, pamidronate disodium, PAN-90806, Panavir I, panobinostat, pantoprazole, pantothenic acid, pantovigar, papaverine, paquinimod, paracetamol, pardoprunox, parecoxib, paricalcitol, paritaprevir, parnaparin sodium, parogrelil, paromomycin, paroxetine, paroxetine hydrochloride, paroxetine hydrochloride hemihydrate, paroxetine mesylate, parthenolide, Passiflora incarnata, patidegib, patiromer calcium, patupilone, pazopanib, pazufloxacin, pazufloxacin mesylate, PCI-24781, PCI-27483, PD-110843, PD-115934, pectin, pefcalcitol, peficitinib, pegamotecan, pegcantratinib, pegylated liposomal doxorubicin, PEITC, pelitinib, pelitrexol, pelubiprofen, pemafibrate, pemetrexate, pemetrexed, pemetrexed disodium, pemirolast, pemirolast sodium, pemoline, penciclovir, penclomedine, penehyclidine hydrochloride, penicillamine, penicillin, penicillin G, penicillin V, pentaerythritol tetranitrate, PentaLyte, pentamidine, pentamidine isethionate, pentazocine, pentobarbital, pentosan polysulphate sodium, pentostatin, pentothal, pentoxifylline, pentoxyphilline, peramivir, perchlozone, peretinoin, perflubron emulsion, perflutren lipid microsphere, pergolide, perhexiline, perifosine, perillyl alcohol, perindopril, perindopril arginine, permethrin, perospirone, perphenazine, perzinfotel, pethidine, pethidine hydrochloride, petrolatum, pexacerfont, pexidartinib, PF-04447943, PF-05089771, PF-05175157, PF-3654746, PF-3654764, PF-4191834, PF-4531083, PF-4691502, PF-489791, PF-610355, PG-2, PGL-2001, PGP/BCRP inhibitor, PH-797804, phenelzine, phenindione, pheniramine maleate, phenobarbital, phenobarbital sodium, phenoxybenzamine, phenprocoumon, phenserine, phentermine, phentolamine mesylate, phenylbutyrate, phenylephrine, phenylephrine hydrochloride, phenytoin, phloroglucinol, PHN-031, PHN-033, phosphate, phosphatidyl serine, phosphatidylcholine, phosphatidylcholine-associated naproxen, phosphatidylcholine-encapsulated ibuprofen, Phospho-Lax, phosphorus, photopheresis, physostigmine, phytate, phytonadione, phytosterols, piboserod, pibrentasvir, picibanil, piclidenoson, piclozotan, picoplatin, picotamide, picroliv, picropodophyllin, picrorhiza, pictilisib, pilaralisib, pilocarpine, pilocarpine hydrochloride, pilsicainide, PIM-447, pimagedine, pimasertib hydrochloride, pimavanserin, pimecrolimus, pimodivir, pimozide, pindolol, pinocembrin, pioglitazone, pioglitazone hydrochloride, pipamperone, piperacillin, piperacillin sodium, piperacillin-tazobactam, piperaquine, piperaquine phosphate, piperine, piracetam, piragliatin, pirarubicin, pirenzepine, pirfenidone, piribedil, piridoxine, piritramide, piritrexim, piromelatine, piroxicam, pitavastatin, pitavastatin calcium, pitolisant, pivmeciellinam, pivmecillinam, pixantrone, PL-3994, plant sterols, platinum, plazomicin, pleconaril, plerixafor, plevitrexed, plinabulin, PLX-8394, PM-00104/50, PMI-001, PMK-N02RS1, pocapavir, polaprezinc, policosanol, polidocanol, polifeprosan 20 with carmustine, polmacoxib, polyethylene glycol, polyethylene glycolated IL-2, polyethylene glycol-citrate-simethicone, polymeric nanoparticle docetaxel, polymyxin B sulphate, polyphenon E, polysaccharide-K, polysorbate 80, polysporin, polytetrafluoroethylene, pomaglumetad methionil, pomalidomide, ponatinib, ponesimod, poractant alpha, porfimer sodium, porfiromycin, posaconazole, posizolid, Posterisan akut, potassium, potassium canrenoate, potassium chloride, Potassium iodide, potassium nitrate, Potassium perchlorate, povidone, povidone iodine, pozanicline, poziotinib, PPA Lux 680, PPA-904, PPD-10558, PPI, PR-104, pracinostat, pradefovir, pradigastat, pralatrexate, pralidoxime, pralnacasan, pramiconazole, pramipexole, pranlukast, pranlukast hydrate, prasterone, prasugrel, pravastatin, praziquantel, prazosin, prednicarbate, prednisolone, prednisolone acetate, prednisolone phosphate, prednisolone sodium metazoate, prednisolone sodium succinate, prednisone, pregabalin, pregnenolone, preladenant, Premarin, Prempro, Prenatal vitamin, presatovir, pretomanid, prilocaine, primaquine, prinaberel, prinomastat, pritelivir, probenecid, Probiotics, probucol, procainamide, procaine, procaine hydrochloride, procarbazine, procarbazine hydrochloride, procaterol, procaterol hydrochloride, prochlorperazine, Procysteine, pro-docosapentaenoic acid, pro-eicosapentaenoic acid, progesterone, progestin, progestogen, progestogen dienogest, proglumide, proguanil, proguanil hydrochloride, Prolarix, promethazine, Prometra, Prometrium, Promisan, propacaine hydrochloride, propacetamol, propafenone, propafenone-SR, propanolol, proparacaine, propionyl-L-carnitine, propiverine, propiverine hydrochloride, propofol, Propofol Lipuro, propofol-lipuro, propranolol, propranolol hydrochloride, propranolol LA, propranolol XL, propylthiouracil, propyphenazone, Prosorba, prostaglandin, prostaglandin-E2, Prostin, Protelos, protriptyline, ProvideXtra, proxymetacaine, proxymetacaine hydrochloride, PRS-211375, prucalopride, prulifloxacin, Prurisol, pruvanserin, PRX-3140, PRX-8066, PSD-508, pseudoephedrine, pseudoephedrine hydrochloride, Pseudomonas aeruginosa mannose-sensitive hemagglutinin (PA-MSHA), PSI-5004, PSI-938, Psyllium powder, PTH, p-toluene sulfonamide, puerarin, puerarin sodium phosphate, pumosetrag, PVAC, PVP-ILH liposomes, PX-12, PYN-17, pyrantel-oxantel, pyrazinamide, pyridostigmine bromide, pyridoxal, pyridoxamine dihydrochloride, pyridoxine, pyridoxine hydrochloride, pyrimethamine, pyrimethamine/sulfadoxine, pyrimethamine/sulphadoxine, pyronaridine, Q-301, QAV-680, Qinbudan, Qizhitongluo, quercetin, quetiapine, quetiapine fumarate, quetiapine IR, quinacrine, quinagolide hydrochloride, quinapril hydrochloride, quinfamide, quinidine, quinine, quinolone, quinolones, quinupristin, quisinostat, quizartinib dihydrochloride, R-112, rabacfosadine, rabeprazole, rabeximod, rabusertib, racecadotril, raclopride, radafaxine, radalbuvir, radequinil, radezolid, Radha-108, radioactive iodine, Radioiodine, radiolabeled iodobenzamide, radiprodil, radium Ra 223 dichloride, radotinib, ragaglitazar, ralfinamide, raloxifene, raltegravir, raltitrexed, ramatroban, ramelteon, ramipril, ramosetron, ranibizumab, ranimustine, ranirestat, ranitidine, ranolazine, rapamycin, rasagiline, raseglurant, ravidasvir hydrochloride, ravuconazole, raxatrigine, razupenem, RBP-8000, RBx-10017609, RDEA-806, RDP-58, rebamipide, rebaudioside A, rebimastat, reboxetine, refametinib, reformulated diclofenac, reformulated mebendazole, regadenoson, regorafenib, regrelor, relebactam, relenopride, relugolix, remifentanil, remimazolam, remimazolam tosylate, remogliflozin etabonate, REN-1654, renzapride, repaglinide, reparixin, repinotan, repurposed ajulemic acid, repurposed ondansetron, resatorvid, reserpine, resiniferatoxin, resminostat, Resoferon, Respifor, Respimat, Restylane, Restylane SubQ, resveratrol, retagliptin, retapamulin, retaspimycin, retigabine, retinoic acid, retinoid, retinol, retosiban, retrovir, revamilast, revefenacin, revexepride, reviparin sodium, rezatomidine, RF-07026, rFSH, RG-4929, RG-7234, RG-7795, RGH-478, RGH-507, Rhenium-188-HEDP, Rhinox, RhuDex, ribavirin, ribociclib, riboflavin, ridaforolimus, ridinilazole, rifabutin, rifalazil, rifampicin, rifampin, rifamycin, rifapentine, rifaximin, rigosertib sodium, rikkunshito, rilapladib, rilmenidine, rilpivirine hydrochloride, riluzole, rimacalib, rimegepant, rimexolone, rimonabant, Ringer's acetate, Ringer's lactate solution, riociguat, ripasudil hydrochloride hydrate, risedronate sodium, risperidone, ritanserin, ritobegron ethyl ester hydrochloride, ritodrine, ritonavir, rituximab, rivaroxaban, rivastigmine, rivenprost, rivipansel sodium, rivoglitazone, rizatriptan, RLP-068, RNF43, RNS-60, RO-4929097, RO-5036505, robalzotan, rociletinib, rocuronium, rocuronium bromide, rofecoxib, Roferon-A, roflumilast, rolapitant, rolipram, rolofylline, ronacaleret, roniciclib, ronopterin, ropinirole, ropinirole hydrochloride, ropivacaine, roquinimex, rose bengal sodium, rosiglitazone, rosiglitazone maleate, rosiglitazone XR, rosiptor acetate, rostafuroxin, rostaporfin, rosuvastatin, rosuvastatin calcium, rotigotine, rovatirelin, roxadustat, roxithromycin, RP-323, RPh-201, RPL-554, RPM-02/08, RQ-00000004, R-salbutamol sulphate, rubitecan, ruboxistaurin, rucaparib, rucaparib camsylate, rucaparib phosphate, rufinamide, rupatadine, ruxolitinib, S Ketamine, S(+)-Ketamine, S-1, S-111, S-38093, S-707106, SAB-378, sabarubicin, Saccharomyces boulardii, sacubitril, S-adenosyl methionine, SAF-312, saffron, safinamide, safotibant, sagopilone, salbutamol, salbutamol HFA, salbutamol sulphate, salicylic acid, salicylic acid+benzoic acid, salidroside, saline, salirasib, salmon calcitonin, salsalate, Salubrin, samarium(153Sm) lexidronam, samatasvir, samidorphan, S-amlodipine gentisate, sapacitabine, sapanisertib, sapitinib, sapropterin, sapropterin dihydrochloride, saquinavir, SAR-110894, saracatinib, sarecycline, saredutant, sargramostim, sarizotan hydrochloride, saroglitazar, sarpogrelate hydrochloride, satavaptan, satraplatin, saxagliptin, SB-773812, SBP-002, SC-49483, SCH-002063, SCH-497079, SCH-900776, schisandra sphenanthera extract, scopolamine, SCY-078, SDX-101, secnidazole, Sedlitzia rosmarinus, segesterone acetate, seladelpar L-lysine dihydrate, selegiline, selegiline hydrochloride, selenium, selenium sulfide, selenomethionine, selepressin, seletracetam, selexipag, seliciclib, selinexor, selisistat, selodenoson, selonsertib, Selozok, selumetinib, selurampanel, semagacestat, semapimod, semaxanib, sembragiline, senicapoc, senna, Sensorcaine, Sentra PM, seocalcitol, sepantronium bromide, sepetaprost, sepranolone, septin, SER-150-DN, Serenoa repens, sergliflozin etabonate, serlopitant, serotonin reuptake inhibitors, serotonin/norepinephrine reuptake inhibitors, sertaconazole, sertindole, sertraline, S-ethylisothiourea diethylphosphate, setileuton, setipiprant, S-etodolac, setrobuvir, sevelamer carbonate, sevelamer hydrochloride, sevoflurane, Sevofran, sevuparin sodium, SG-2000, Shanhuang Wuji decoction, SHP-465, sibrafiban, sibutramine, sildenafil, sildenafil citrate, Silexan, stin, silibinin dihydrogensuccinate, silimarine, silodosin, silver nanoparticle, silver nitrate, silver sulfadiazine, silybin, silymarin, simenepag isopropyl, simeprevir, simethicone, simeticone, simvastatin, Sinbaro, siponimod, sirolimus, sitafloxacin, sitagliptin, sitamaquine, sitaxentan, sitosterol, sivelestat, sivifene, s-ketamine, SKP-1052, SK-PC-B70M, SLx-4090, SMANCS, S-methionyl-L-citrulline, smilagenin, SMP-028, SN-38, SNX-5422, sodelglitazar, sodium, sodium 4-phenylbutyrate, sodium ascorbate, sodium benzoate, sodium bicarbonate, sodium butyrate, sodium carboxymethylcellulose, sodium chloride, sodium chromoglycate, sodium citrate, sodium dichloroacetate, sodium ferric gluconate complex, sodium fluoride, sodium folinate, sodium fusidate, sodium hyaluronate, sodium hydroxide, sodium hypochlorite, sodium ketorolac, sodium lactate, sodium nitrate, sodium nitrite, sodium nitroprusside, sodium oxybate, sodium phenylacetate, sodium phenylbutyrate, sodium phosphate, sodium picosulphate hydrate, sodium polystyrene sulfonate, sodium prasterone sulphate, sodium pyruvate, sodium stibogluconate, sodium sulfide, sodium tetradecyl sulphate, sodium thiopental, sodium thiosulphate, sodium valproate, sodium-fluoride, sofinicline, sofosbuvir, sofosbuvir+daclatasvir, sofpironium bromide, solabegron, solcitinib, Soldesam, solifenacin, solithromycin, soluble ferric pyrophosphate citrate, Solvazinc, somatostatin, sonedenoson, sonidegib, sonolisib, sorafenib, soraprazan, sorbitol, sorivudine, sotagliflozin, sotalol, sotrastaurin, sovaprevir, soy isoflavones, sparsentan, spebrutinib, spiramycin, spironolactone, sPLA2 inhibitors, SQ-109, squalamine, SR-T100, SSS, ST-101, STA-4783, standard bicarbonate, standard pneumoperitoneum, stannous compounds, stannsoporfin, statin, stavudine, stearidonic acid, S-tenatoprazole, Sterofundin, steroid, steroids, stiripentol, Stopain, Streptococcus faecium, Streptococcus thermophillus, streptomycin, streptozocin, strontium chloride Sr 89, strontium malonate, strontium ranelate, strontium-89, STW-5, STX-107, SU, SU-101, SU-14813, succimer, succinic acid, succinylcholine, sucralphate, sucralose, sucroferric oxyhydroxide, sucrose, Sufenta, sufentanil, sufentanil citrate, sufentanyl, sugammadex, sulbactam, sulbactam sodium, sulfadiazine, sulfadoxine, sulfadoxine+pyrimethamine, sulfalene-pyrimethamine, sulfamethoxazole, sulfasalazine, sulphate salt solution, sulfonyl urea, sulfonylurea, sulfonylureas, sulforaphane, sulindac, sulodexide, sulopenem, sulopenem etzadroxil, sulphacetamide sodium, sulphadoxine, sulphadoxine-pyrimethamine, sulphamethoxazole, sulphonylurea, sulpiride, sultamicillin, sumanirole, sumatriptan, sumatriptan succinate, SUN-0597, SUN-1334H, sunitinib, suplatast tosilate, suramin, suramin sodium, surinabant, sutezolid, suxamethonium, SYI-2074, symbiotic, synbiotics, SYNSORB-Pk, synthetic hypericin, TIS, T-1225, T-2000, T3, tacalcitol, tacedinaline, tacrine, tacrolimus, Tacrolimus Hexal, tadalafil, tadekinig alpha, tafamidis, tafenoquine, tafluprost, tafoxiparin sodium, TAK-715, TAK-783, talabostat, taladegib, talampanel, talaporfin, talarozole, talazoparib, talc, TALL-104, talmapimod, talnetant, talniflumate, talotrexin, talsaclidine fumarate, taltirelin, tamibarotene, tamoxifen, tamsulosin, tamsulosin hydrochloride, tandospirone, tandutinib, tanespimycin, tanomastat, tanzisertib, tapentadol, taprenepag, Taradyal, tarafenacin, taranabant, tarenflurbil, taribavirin hydrochloride, tariquidar, tarloxotinib bromide, tasidotin HCl, tasimelteon, tasisulam, taspoglutide, tasquinimod, taurolidine, tauroursodeoxycholic acid, tavaborole, tavilermide, Taxol, Taxus, tazarotene, tazobactam, TC-2403, TC-3, TCM-606F, tebipenem pivoxil, tecadenoson, tecalcet, tecarfarin, tecastemizole, technetium bicisate, technetium etarfolatide, technetium Tc 99m tilmanocept, technetium Tc 99m trofolastat, technetium-99, tecovirimat, tedatioxetine, tedisamil, tedizolid phosphate, tegafur, tegaserod, teglarinad chloride, tegobuvir, teicoplanin, telaprevir, telapristone acetate, telatinib, telbivudine, telcagepant, telithromycin, telmisartan, telotristat etiprate, temazepam, temocapril, temocillin, temoporfin, temozolomide, temsirolimus, tenapanor, teneligliptin, teniposide, tenofovir, tenofovir alafenamide, tenofovir alafenamide fumarate, tenofovir disoproxil fumarate, tenofovir exalidex, tenofovir/emtricitabine, tenoxicam, teprenone, terameprocol, terazosin, terbinafine, terbinafine gel, terbinafine hydrochloride, terbogrel, terbutaline, terbutaline sulphate, terconazole, terguride, teriparatide, terlipressin acetate, terutroban, tesaglitazar, tesetaxel, tesevatinib, tesmilifene, tesofensine, Testagen, testosterone, testosterone cypionate, testosterone enanthate, testosterone undecanoate, Tetanus toxoid, tetomilast, tetrabenazine, tetracaine, tetracaine hydrochloride, tetracycline, tetracycline HCl, tetrahydrobiopterin, tetrahydrocannabinol, tetrathiomolybdate, Tetrodin, tezacaftor, tezacitabine, tezampanel, tezosentan, TG-100-115, TG-100801, thalidomide, THC, theophyllamine, theophylline, theophylline SR, theracurmin, thiamine, thiamine hydrochloride, thiazide, thiazide diuretics, thiazolidinedione, thiazolidinediones, thiocolchicoside, thioctic acid, thioguanine, thiopental, thiopental sodium, thiopentone, thioridazine, thiotepa, thiothixene, THR-0921, THR-4109, thrombin, thrombin microcapsules, thymoctonan, Thymoglobulin, thyroxine, tiagabine, tianeptine, tiapride, tibolone, ticagrelor, ticlopidine, tideglusib, tigecycline, tilapertin, tilarginine acetate, tiludronate disodium, timapiprant, timcodar, timnodonic acid, timolol, timolol, timolol maleate, tinidazole, tinzaparin sodium, tiopronin, tiotropium, tiotropium bromide, tipelukast, tipifarnib, tipiracil hydrochloride, tipranavir, tirapazamine, tirasemtiv, tirilazad, tirofiban, tirofiban hydrochloride, titanium dioxide, TIVA, tivantinib, tivozanib, tizanidine, TMC-114/RTV, TMC-310911, TMC-647055, TNF-alpha, TNP-470, tobramycin, tocladesine, tocofersolan, tocopherol, tocopherols, tocopheryl phosphate mixture (TPM), tofacitinib, tofimilast, tofogliflozin, tolcapone, tolevamer, tolnaftato, tolperisone, tolterodine, tolterodine tartrate, tolvaptan, TOMM34, tonabersat, Tonalin SG1000T FFA, tonapofylline, Tongxinluo, topiramate, topiroxostat, topotecan, topotecan hydrochloride, torasemide, torcetrapib, toreforant, toremifene, tosedostat, tosufloxacin, tozadenant, tozasertib, trabectedin, trabectome, trabodenoson, tradipitant, tramadol, tramadol hydrochloride, tramazoline, trametinib, tramiprosate, trandolapril, tranexamic acid, tranilast, transcrocetinate-sodium, tranylcypromine, trastuzumab, travoprost, traxoprodil, trazadone, trazodone, trazodone hydrochloride, TRC-101, trehalose, trelagliptin succinate, trelanserin, treosulfan, treprostinil, treprostinil diolamine, tretinoin, triamcinolone, triamcinolone acetonide, triamcinolone hexacetonide, Triapine, Triatec HCT, Triaz, triazavirin, triazolam, tribendimidine, trichlormethiazide, trichlorothiazide, triciribine, tridolgosir, trientine, trientine hydrochloride, trifarotene, trifluoperazine, trifluridine, triflusal, triheptanoin, trihexyphenidyl, triiodothyronine, trilostane, trimebutine, trimebutine 3-thiocarbamoyl-benzenesulfonate, trimegestone, trimetazidine, trimethaphan, trimethobenzamide, trimethoprim, trimethoprim-sulfa, trimetrexate, trimipramine, trimipramine maleate, trinitrate, Triomune, Triplixam, tripotassium dicitrate bismuthate, tripterygium wilfordii, triptorelin acetate, triptorelin pamoate, trisodium citrate dihydrate, Trivax-AD, trofinetide, trofosfamide, troglitazone, tropicamide, tropisetron, trospium chloride, troxacitabine, troxipide, Trunature, trypsin-EDTA, TS-022, TTK, TTP-054, TTP-399, TTP-435, TTP-889, TTX-9401, tucaresol, tucidinostat, tulobuterol, TV-46763, TV-5010, TY-51924, tylenol, TZD, TZP-102, ubidecarenone, ubiquinol, ubrogepant, UCA-001, UCA-002, UCN-01, udenafil, UFT, UFT/LV, UFUR, UISH-001, UK-390957, UK-432097, UK-447841, ulimorelin, ulinastatin, ulipristal, ulobetasol, ulodesine, umeclidinium, umeclidinium bromide, umirolimus, unfractionated heparin, uniphyllin, upadacitinib, upamostat, uprifosbuvir, uprosertib, UR-906, uracil, urapidil, urea, uridine, uridine triacetate, URLC10, ursodeoxycholic acid, Ursolic acid, Urtica diociea, usistapide, UTD-1, utrogestan, V-116517, V-158866, V-404, VA-111913, vabicaserin, vaborbactam, vadadustat, vadimezan, Vagiprost, VAK-694, valaciclovir, valacyclovir, valbenazine, valdecoxib, valerian extract, valganciclovir, valium, valnivudine hydrochloride, valnoctamide stereoisomers, valomaciclovir stearate, valopicitabine dihydrochloride, valproate, valproic acid, valrocemide, valrubicin, valsartan, valsartan trisodium hemipentahydrate, valspodar, valtorcitabine, vancomycin, vancomycin hydrochloride, vandetanib, vaniprevir, vanoxerine, vapendavir, vapitadine, vardenafil hydrochloride, varenicline, varespladib, varespladib methyl, varlitinib, vasopressin, vatalanib, vatiquinone, VDC-2008, vecuronium, vecuronium bromide, vedroprevir, VEGFR1, VEGFR1 peptide, VEGFR1-A02-770, VEGFR2 peptide, VEGFR2-derived HLA-A0201, veliflapon, veliparib, velneperit, velpatasvir, velusetrag, vemurafenib, venetoclax, venlafaxine, venlafaxine hydrochloride, venlafaxine XR, vepoloxamer, verapamil, verapamil hydrochloride, vercirnon, verdiperstat, vericiguat, verinurad, vernakalant, vernakalant hydrochloride, verteporfin, verubulin, verucerfont, Verutex, vesatolimod, vesnarinone, vestipitant, vibegron, vicriviroc, Vidoca, vidofludimus, vidupiprant, vigabatrin, viitamin D, vilanterol, vilanterol trifenatate, vilaprisan, vilazodone, vildagliptin, vinblastin, vinblastine, vinblastine sulphate, vincristin, vincristine, vincristine sulphate, vindesine, vinflunine, vinorelbine, vinorelbine ditartrate, vinpocetine, vintafolide, vipadenant, vismodegib, vistusertib, Vit B12, Vitamac, vitamin A, vitamin B, vitamin B1, vitamin B12, vitamin B-12, vitamin B2, vitamin B6, vitamin B-6, vitamin C, vitamin D, vitamin D2, vitamin D3, vitamin E, vitamin E succinate derivatives, vitamin K, vitamin K1, vitamins B1, vitamins B12, vitamins B2, vitamins B6, Vitreosolve, Viusid, Vizomitin, vofopitant, voglibose, volasertib, volinanserin, vonoprazan fumarate, vorapaxar, voriconazole, vorinostat, vortioxetine hydrobromide, vosaroxin, voxilaprevir, voxtalisib, VP-101, VRx-3996, VSL-3, WZ-149, VX-105, VX-135, VX-702, VX-710, Wafermine, warfarin, warfarin sodium, Water, WF-10, WH-1, Wobenzym, WX-554, xaliproden, xemilofiban, xenon, Xiang-sha-hu-jun decoction, xiaoqinglong, Xibrom, Xilei-San, ximelagatran, Xiyanping, XL-139, XP-21279, Xylitol, xylometazoline, Y-39983, Yallaferon, yonkenafil, yttrium clivatuzumab tetraxetan, yttrium Y 90 anti-CD66 monoclonal antibody BW 250/183, zabofloxacin, zafirlukast, zalcitabine, zaleplon, zaltoprofen, zanamivir, ZD-6126, zeaxanthin, zibotentan, zicronapine, zidovudine, zileuton, zinc, zinc acetate, zinc oxide, zinc picolinate, zinc sulphate, zinc supplement, Zincas Forte, Zinthionein, ziprasidone, zofenopril, zoledronate, zoledronic acid, zoliflodacin, zolmitriptan, zolpidem, zolpidem tartrate, Zometa, zonampanel, zonisamide, zonisamide SR, zopiclone, zosuquidar, zotarolimus, zotepine, zucapsaicin, zuclopenthixol, and zuretinol acetate.

In certain embodiments, a stimulus that perturbs a GSC is a small molecule selected from the CeMM Library of Unique Drugs (CLOUD), as shown in Licciardello et al., Nat Chem Bid; Vol. 13, pages 781-780 (2017). For example, the small molecule is selected from: Pinacidil, Altretamine, Pipobroman, Uracil Mustard, Trioxsalen, Plicamycin, Ambenonium, Edrophonium, Hexafluorenium, Oxtriphylline, Arbutamine, Guanabenz, Mephentermine, Methoxamine, Phenylpropanolamine, Protokylol, Tetrahydrozoline, Tolazoline, Bethanidine, Ergoloid, Oxprenolol, Penbutolol, Phentolamine, Propiomazine, Thiethylperazine, Fomepizole, Triamterene, Stanozolol, Dromostanolone, Ethylestrenol, Fluoxymesterone, Methyltestosterone, Deserpidine, Quinapril, Rescinnamine, Spirapril, Testolactone, Ethionamide, Sulfameter, Sulfacytine, Sulfamerazine, Sulfamethazine, Sulfamethizole, Sulfaphenazole, Sulfapyridine, Sulfathiazole, Sulfisoxazole, Sulfoxone, Cefmenoxime, Amdinocillin, Azlocillin, Bacampicillin, Carbenicillin, Cefalotin, Cefamandole, Cefditoren, Cefonicid, Ceforanide, Cefotiam, Cefpiramide, Cefradine, Ceftizoxime, Cephaloglycin, Cephapirin, Cyclacillin, Hetacillin, Loracarbef, Methicillin, Mezlocillin, Moxalactam, Nafcillin, Ticarcillin, Capreomycin, Demeclocycline, Dirithromycin, Methacycline, Oxytetracycline, Spectinomycin, Troleandomycin, Viomycin, Enoxacin, Novobiocin, Alatrofloxacin, Cinoxacin, Lomefloxacin, Nalidixic Acid, Sparfloxacin, Trovafloxacin, Acetohydroxamic Acid, Marinol, Ethoxzolamide, Acetohexamide, Fenoprofen, Oxyphenbutazone, Carprofen, Oxaprozin, Phenylbutazone, Tolmetin, Meclofenamic Acid, Methylergonovine, Acetophenazine, Carphenazine, Chlorprothixene, Mesoridazine, Triflupromazine, Promazine, Benzphetamine, Phenmetrazine, Chlorotrianisene, Estrone, Mestranol, Polyestradiol, Quinestrol, Cortisone, Fluprednisolone, Meprednisone, Paramethasone, Oxamniquine, Azatadine, Bromodiphenhydramine, Buclizine, Carbinoxamine, Chlophedianol, Dexbrompheniramine, Diphenylpyraline, Mepyramine, Methdilazine, Trimeprazine, Tripelennamine, Triprolidine, Romidepsin, Primidone, Butabarbital, Chlormezanone, Flurazepam, Glutethimide, Halazepam, Meprobamate, Metharbital, Methyprylon, Prazepam, Quazepam, Secobarbital, Talbutal, Thiamylal, Gamma Hydroxybutyric Acid, Memantine, Triclofos, Piperazine Hexahydrate, Desoxycorticosterone Pivalate, Pargyline, Carbachol, Oxyphenonium Bromide, Anisotropine, Clidinium, Cycrimine, Dicyclomine, Diphemanil, Ethopropazine, Fesoterodine, Hexocyclium, Isopropamide, Mepenzolate, Methantheline, Methylscopolamine, Metixene, Orphenadrine, Oxyphencyclimine, Procyclidine, Propantheline, Tridihexethyl, Trospium, Decamethonium, Pentolinium Tartrate, Cisatracurium Besylate, Succinylcholine Chloride, Doxacurium, Gallamine, Metocurine, Pancuronium, Pipecuronium, Rapacuronium, Tubocurarine, Guanadrel, Phendimetrazine, Anileridine, Difenoxin, Diphenoxylate, Levomethal, Oxymorphone, Propoxyphene, Levallorphan, Methylnaltrexone, (4)-Sulfinpyrazone, Pamidronic Acid, Risedronate, Tiludronate, Clofibrate, Dyphylline, Inamrinone, Vardenafil, Ethynodiol, Hydroxyprogesterone, Norethynodrel, Ulipristal Acetate, Carboprost, Etretinate, Methysergide, Chlorphentermine, Acetyldigitoxin, Deslanoside, Chlorpropamide, Tolazamide, Tolbutamide, Methyclothiazide, Benzthiazide, Chlorothiazide, Cyclothiazide, Hydroflumethiazide, Polythiazide, Quinethazone, 5-Fluorouracil, Dextrothyroxine, Metyrosine, Rimantadine, Adefovir, Anisindione, Dicumarol, Nisoldipine, Trimethadione, Bepridil, Paramethadione, Bretylium Tosylate, Mephenytoin, Benzonatate, Ethotoin, Indecainide, Moricizine, Phenacemide, Tocainide, Pyrvinium Chloride Dihydrate, Halofantrine, Metaxalone, Diphenidol, Mebutamate, Chlorphenesin, Phensuximide, Thiabendazole, Benzquinamide, Piperacetazine, Ethchlorvynol, and Ethinamate.

In some embodiments, a biological may be used in lieu of a small molecule in the above-described list. In some embodiments, an herbal preparation may be used in lieu of a small molecule in the above-described list. In some embodiments, a borderline product may be used in lieu of a small molecule in the above-described list.

### Known Drugs

In certain embodiments, a stimulus that perturbs a GSC is a drug selected from: (11C) acetate, (11C)-PHNO, (13C) sodium acetate, (18F) fluorothymidine, (2 beta-carbomethoxy-3 beta-[4-iodophenyl] tropane), [1-13C] leucine, [1-13C] NaHCO3, [11C] Cimbi-36, [11C](R)-PK11195, [11C]-(S)-ketoprofen methyl ester, [11C]-K-2, [11-C]methionine, [11C]-raclopride, [123I] 5-IA, [123I] iodobenzamide, [18]FDG, [18F] fallypride, [18F]-Fluoromisonidazole, [18F]-CP18, [18F]-FDG, [18F]Fluoro-2-deoxy-2-D-glucose, [18F]-fluorodeoxyglucose, [18]-fluorodeoxythymidine, [18F]fluorothymidine, [F-18] fluorodeoxyglucose, [F-18] FMISO, 1-(Di-isopropyl-phosphinoyl)-nonane, 1,2 dithiolane 3 valeric acid, 1,5-pentanediol, 1-[13C]-leucine, 11C choline, 11C-5-HTP, 11C-acetate, 11-carbon-choline, 11C-choline, 11C-L-deprenyl-D2, 11C-metomidate, 123-I iodobenzovesamicol, 125I-albumin, 131 iodine, 13C Methacetin, 13C Sodium Octanoate, 13C-dextromethorphan, 13-cis-retinoic acid, 13C-labeled methacetin, 13C-Methacetin, 13C-uracil, 150-water, 16-18-F-fluoro-4-thiapalmitate, 16 alpha-[18F] estradiol, 16-alpha-[18-fluoro]-17betaestradiol, 17 hydroxyprogesterone caproate, 17-b-estradiol, 17-beta-estradiol, 17-beta-estradiol/dydrogesterone, 17-hydroxyprogesterone caproate, 18 F-fluorothymidine, 1-84 parathyroid hormone, 188Re-sulfur colloid, 18F, 18F choline, 18F fallypride, 18F fluorodeoxyglucose, 18F Fluoromisonidazole, 18F fluorothymidine, 18F-2-deoxy-2-fluoro-D-glucose, 18F-2-fluoro-2-deoxy-D-glucose, 18F-choline, 18F-deoxyglucose, 18-FDG, 18F-DOPA, 18F-DTBZ, 18F-ethylcholine, 18F-FDG, 18F-flouro-deoxyglucose, 18F-FLT, 18F-flumazenil, 18F-fluoride, 18F-fluoro-3′-deoxy-3-L-fluorothymidine, 18F-fluoroazomycin arabinoside, 18F-fluorocholine, 18F-fluorodeoxy-d-glucose, 18F-fluorodeoxyglucose, 18F-fluorodihydrotestosterone, 18F-fluoroestradiol, 18F-fluoro-L-thymidine, 18F-fluoromethylcholine, 18F-fluoromisonidazole, 18F-fluorothymidine, 18F-FMISO, 18F-labeled fluorodeoxyglucose, 18F-labelled HX4, 18-fluorodeoxyglucose, 18-fluoro-deoxy-glucose, 18-fluorodeoxyglucose/F-18 FDG, 18F-MISO, 18-NaF, 1-deamino-8-D-arginine, 1F-fluoro-ethyl-tyrosine, 1-octanol, 2[fluorine-18]fluoro-2-deoxy-d-glucose, 25 vitamin D, 25-hydroxyvitamin D, 2-aminoethanesulfonic acid, 2-chloroprocaine, 2-ClinOleic, 2-deoxy-2-[18F] fluoro-D-glucose, 2-octylcyanoacrylate, 3,4-methylenedioxymethamphetamine, 3,4-methylenedioxymethamphetmine, 3,4-methylenedioxymethemphetmaine, 3-deoxy-3∝[18F]-fluorothymidine, 3-deoxy-3-[18F]fluorothymidine, 3-deoxy-3-18F-fluorothymidine, 3-helium gas, 3-hydroxybutyrate, 3M ESPE, 3-O-methylglucose, 4 methylenedioxyamphetamine, 4-aminosalicylic acid, 4-dichlorobenzyl alcohol, 4-hydroxybutanoic acid, 4-methylenedioxymethamphetamine, 4-n-butylresorcinol, 4-terpineol, 5 hydroxy-tryptophan, 5-ALA, 5-amino levulinic acid, 5-aminoimidazole-4-carboxamide ribonucleoside, 5-aminolevulinic, 5-aminolevulinic acid, 5-aminosalicylic acid, 5-hydroxy tryptamine, 5LGr, 5-Loxin, 5-methyltetrahydrofolate, 6,6-[2H2]glucose, 62Cu-ethylglyoxal bis, 64Cu-ATSM, 68Ga-alphatide 11, 68Ga-DOTA-[3-Tyr]-octreotide, 68Ga-DOTANOC, 68-Ga-DOTANOC, 68Ga-PSMA, 6-alpha-(18F)fluoro-17 beta-estradiol, 6-DIG, 6-mercaptopurine, 82rubidium, 8-methoxypsoralen, 90 Yttrium spheres, 99 m Technetium, 99 m Technetium-labeled albumin, 99mTc, 99mTc sulfur colloid, 99mTc-albumin, 99mTc-labeled nanocolloidal albumin, 99mTc-radiolabelled tiotropium, 99mTc-sestamibi, 99mTc-sulfur colloid, 9-d-tetra hydro cannabinol, A4I Antagonist, abacavir, ABH, Abnoba Viscum F, abnobaVISCUM Fraxini, AboMeg-B-09 syrup, Absinthium, AC-1204, Acacia catechu, acamprosate calcium, Acanya, acarbose, acebrophylline, acebutolol, aceclofenac, ACE-inhibitor, acenocoumarol, Acetadote EF, acetaminophen, acetate, acetate mafenide, acetazolamide, acetazolamide acetate, acetic acid, acetylcholine, Acetyl-hexapeptide-8, acetyl-L-carnitine, acetylsalicylic acid, acetylsalicylic acid lysinate, acetyl-salicylicum, aciclovir, acipimox, acitretin, aclidinium, AcSDKP-NH2 Cr-EDTA, AcSDKP-NH2 inuline, ActaVisc, ActaVisc Mx, Actinidia arguta extract, activated carbon, Activomin, Actovegin, ACYC, acyline, Ad2/HIF-1aNP16, Adamin-G, adapalene, adcal-D3, Addyzoa, adefovir dipivoxil, adenosine, adenosine 5′-triphosphate, adenosine-5′-monophosphate, adomiparin, adrenalin, adrenaline, adrenocorticotropic hormone, adriamycin, Advil, gentamicin, Afalaza, Aflapin, Aflubin, Aflurax, Aftogel, AG-01, AG-05, AG-06, AG-07, Ageratum conyzoides, agomelatine, Ajwain, AL-817, alagebrium, Alamin SN, alanylglutamine, alanyl-glutamine-dipeptide, Alaria esculenta, albendazole, albumin, Albutein, albuterol, albuterol HFA, albuterol/salbutamol, alcaftadine, Alcaine, alclometasone, alcohol, alendronate, alendronate sodium, alendronate/cholecalciferol, alendronic acid, alphacalcidiol, alphacalcidol, alfentanil, alginic acid, alhydran, Alimax, alisertib, aliskiren, Allegra, Allergovit, Allerslit forte, allopurinol, alosetron, alpha lipoic acid, alpha methyl-para-tyrosine, alpha/beta-arteether, alpha-[11C]methyl-L-tryptophan, alpha-keto acid, alpha-linolenic acid, alpha-lipoic acid, alpha-methylnoradrenaline, Alpha-Tocopherol, alprazolam, Alprostadil, Atim, aluminium based phosphate binder, aluminum hydroxide, alvocidib, amantadine, amantadine sulphate, Amargol, ambrisentan, ambroxol, ambulatory oxygen, Amerigel, amethocaine, AMI-25, amibegron, amifostine, amikacin, amikacin sulphate, aminaftone, amine fluoride, amine fluoride/stannous fluoride, aminexil, amino acids mixture, Aminoleban EN, aminomidazole carboxamide riboside, aminophylline, aminosalicylic acid, amiodarone, amithiozone, amitriptyline, amitriptyline hydrochloride, amlodipine, amlodipine besylate, amlodipine/valsartan, amlodopine, ammonia, Amnioexcel, AmnioFix, amodiaquine, amorolfine, amoxicillin, amoxycillin, Amphadase, amphetamine, Amphomul, Amphora, amphotericin B, amphotericin B deoxycholate, ampicillin, amprenavir, Amurojipin, amylmetacresol, Anaesterit, anafranil, anagrelide retard, analgecine, anastrozole, Anatabloc, Ancef, Androfeme, AndroForte 5, andrographis, Andrographis paniculata p/st extract, andrographolide, angiotensin, angiotensin amide, angiotensin II, Angipars, anidulafungin, anlotinib, annona muricata, Antara, AnteAGE, anthocyan, anthracycline, Antibiophilus, antibiophilus(Lactobacillus casei), Anti-D, antihypertensive agents, antihypertensives, antimonials, Antinitus, antipyrine, Anti-Rin, antiscorpion serum, Antistax, anti-thymocyte globulin, antithymoglobulin, Antivipmyn, Antiwei granule, Antox, apatinib mesylate, APD-209, APDDR-0901, apelin, apomorphine hydrochloride, Apo-Oxycodone CR, Apotel Max, A-prexa, aprotinin, AR-623, arabinogalactan extract, Arbidol, arbutin, Argamater, argatroban, Arginaid Water, arginine, arginine acetate, arginine hydrochloride, arginine vasopressin, argireline, Aridol, aripiprazole, Arista, Aristocort A, aristospan, armodafinil, Arnica, arsenic trioxide, Arsuamoon, Arsumax, ArtCure, Artelac Rebalance, artemether, artemisinin, artemotil, artenimol, Artequick, Artequin, artesunate, Arthrem, articaine, articaine hydrochloride, Artichoke WPC, Artrox, Artz, AS-01 adjuvant, As4S4, ascorbic acid, ascorbic acid, Askina Calgitrol, asparaginase, asparaginase medac, astaxanthin, Astragalus, Astressin 2B, asunaprevir, atamestane, Atarax, atazanavir, atenolol, Ateronon, ATG, ATG Fresenius, ATG Fresenius S, ATG-F, atomoxetine, Atorva, atorvastatin, atovaquone, atracurium, Atralin, Atrapro, atrasentan, atropine, Augment, Autan, AutoloGel, Avagard, Avocado soya unsaponifiable, axitinib, Ayulax, Ayurved Siriraj Prasaplai, azathioprine, azelaic acid, azelastine, azelastine hydrochloride/fluticasone propionate, azithromycin, bacitracin, Baclofen ER, Bacopa monnieri, Bactigras, Balance, Bangpungtongsung-san, Banlice, BanxiaHoupuTang, barium sulphate, basiliximab, Basiscreme DAC, BAY86-5032, BAY-86-5037, BAY-98-7111, bazedoxifene, BCG, BCN Peptides, beclabuvir, beclometason, beclometasone dipropionate, beclomethasone, beclomethasone dipropionate, Bedicort A, bedrocan, benazepril, Benefiber, Benjakul extract, benoxinate hydrochloride, benserazide, BenzaClin, benzalkonium chloride, benzatropine, benzocaine, benzodiazepines, benzoyl peroxide, benzydamine, benzydamine hydrochloride, benzydamine hydrochloride/cetylpyridinium chloride, benzylpenicillin sodium, beractant, berberine, Berocca Performance, Beta-1,3-glucan, Beta-Aescin, beta-carotene, Betadine, beta-glucosylceramide, betahistamine, betahistine dihydrochloride, Betaine, betaine HCl, beta-lactam, beta-lactam antibiotics, betamethasone, betamethasone acibutate, betamethasone dipropionate, Betamethasone sodium phosphate, betamethasone valerate, betamethasone valerate, betamethasone-ESI, Betaprim, BetaSal Losung, Betaserc, Beta-TCP/HA, Betnoderm, Betnovat, Betnovate, BH4, bhaspa sweda, Biafine, bicalutamide, bicarbonate, BicaVera, Bicillin L-A, Bifidobacteria lactis, Bifidobacterium breve, Bifilact, Bifluorid 10, bifonazole, Bile acids, bimatoprost, bimatoprost, bimatoprost/timolol PF, BIND-014, bioclavid, Biofreeze, Bioglan, Biogun, Bio-K+CL1285, Biolipid B2, bo-three, biotin, BioXtra, Bi-PegLyte, Biphosphonate, Biprol, birinapant, BIS ImpediMe SFB7, bisacodyl, Biseko, bismuth, bismuth subcitrate, bismuth subsalicylate, bisoctrizole, Bisono, bisoprolol, bisoprololo, bisphosphonate, Black Cohosh, Bledilait Biofer, bleomycin, Blephamide, BUS, blonanserin, Blox4, BLR-250, BLR-350, BLVR Hydrogel, bofutsushosan, Bolgre, boric acid, boron phenylalanine, boronophenylalanine-fructose complex, bortesomib, bortezomib, bosentan, bosutinib, Boswelan, Boswellia extract, Boswellia serrrata, Botulaks, bovine calf intestinal alkaline phosphatase, bovine hyaluronidase, bovine lactoferrin, bradykinin, branchamin, branched-chain amino acid, Bricanyl, brilacidin, brimonidine, brimonidine tartrate, brimonidine UD, brincidofovir, brinzolamide, brivanib alaninate, Brizantin, bromelaine, bromocriptine, bronchobarij, Broncho-Vaxom, brushing, Bryophyllum pinnatum, bryostatin-1, BSC, BSL02-150929Y, BSS Plus, BST-DermOn, Buccagel, Budesonida, budesonide, budesonide chlorofluorocarbon, budesonide hydrofluoroalkane, bulaquine, Bulkamid, buparlisib, bupivacaine, Bupivacaine hydrochloride, bupivicaine, buprenorphine, buprenorphine hydrochloride, bupropion, bupropion hydrochloride, bupropion hydrochloride SR, buproprion, burprenorphine, buscopan, Buscopan plus, buserelin, buspirone, busulfan, Busulfex, Butantan, butenyl ALA, butoconazole nitrate, butorphanol tartrate, Buzzy, B-vitamins, C-11 Acetate, C13 cholesterol, c13 methacetin, C-1-esterase, 01-esterase inhibitor, Ca ionophore, cabazitaxel, caffeine, caffeine benzoate, calci soya balance, calcichew D3, calciferol, Calcifidiol, Calcijex, calcipotriol, calcitriol, calcium, calcium acetate, calcium acetate oral, Calcium Alumina-Silicate, calcium based phosphate binder, calcium carbonate, Calcium channel blocker, calcium chloride, calcium citrate, calcium dialysate, calcium disodium EDTA, calcium fructoborate, calcium gluconate, calcium glycerophosphate, calcium hydroxide, calcium ionophore, calcium pantothanate, calcium phosphate bone cement, calcium phosphopeptide-amorphous calcium phosphate, calcium sodium phosphosilicate, calcium supplement, calcium carbonate, calcium-based phosphate binder, Calendula Weleda, Calmagen, Calmoseptine, Calver, Camel Snus, canagliflozin, Canasa, candesartan, candesartan cilexetil, Canephron N, cangrelor, cannabidiol, Cannabis, Canoderm, canrenone, cantharidin, capecitabine, Caphosol, Capilen, Cappra, capreomycin sulphate, capsaicin, captopril, carbabenzpyride, carbamazepine, Carbatin, carbetocin, carbidopa, carbogen, Carbomer, Carbon C 11 alpha-methyltryptophan, carbon dioxde, carbon nanoparticles, carbon-11 acetate, carboplatin, carbosylane, Carbosymag, Carboxymethylcellulose, Carboxymethylcellulose sodium, carfilzomib, cariprazine, carisoprodol SR, carmustine, carni Q-gel, carnosine, caroverine, carraghenates, carteolol hydrochloride, carvedilol, carvedilol phosphate, casein, casein glycomacropeptide, CasiGold, CasiMax, casopitant, caspofungin, Catechin, catecholamine, cathine hydrochloride, CD-1579, CDRI 80/574, Cebes, cefazolin, cefazolin sodium, cefazoline, cefixime, cefmetazole, cefotaxime, cefpodoxime, ceftazidime, ceftriaxone, cefuroxim, cefuroxime, Celadrin, celecoxib, Celestone, Celox, Centella asiatica selected triterpenes, cepacol, cephalexin, cephalozin, cephazolin, ceramide, Ceratonia siliqua, Cerebrolysin, Cernilton, certoparin, cerubidine, Cervitec CHX, Cetaphil, cetirizine, cetirizine dihydrochloride, cetirizine hydrochloride, cetomacrogol, cetrozole, cetuximab, cetylpyridimium chloride, Chakshusya-Rasayana, chamomile, Chamomilla, Chamomilla recutita, Chandrakanthi Choornam, charcoal, ChiNing decoction, Chixiangshen Tongxin, chlorhexidine, chlorhexidine acetate, chlorhexidine digluconate, chlorhexidine gluconate, chlorine dioxide, chlorofluorocarbon beclomethasone dipropionate, chlorogenic acid, chloromycetin, chloroprocaine HCl, chloroquine, chloroquine diphosphate, chloroquine sulphate, chloroquine/proguanil, chloroquinoxaline sulfonamide, chlorpheniramine, chlorproguanil, chlorpromazine, chlorthalidone, cholecalcierol, cholecalciferol, cholecystokinin, cholesterol, Choline, choline bitartrate, choline dihydrogen citrate, chondroitin sulphate, Chondroitin sulphate/Diacerein, chondroitin sulphate-iron colloid, Chondrosulf, Chong Kun Dang, chorionic gonadotropin, chromium, chromium ethylenediaminetetraacetic acid, chromium nicotinate, chromium piccolinate, chromium picolinate, CI-581, cibenzoline, ciclesonide, ciclopirox, ciclopirox olamine, ciclopiroxolamine, ciclosporin, ciclosporin Pro, Cimicifuga racemosa, cinacalcet, cineole, CINGAL, cinitapride hydrogen tartrate ER, cinnamon, cinnarazine, cinnarizine, Clonal, ciprofloxacin, ciprofloxacin hydrochloride, cis-4-hydroxy-L-proline, cisatracurium, cis-atracurium, cisatracurium besilate, cisplatin, citalopram, citalopram hydrobromide, citicoline, Citracal, Citrafleet, citrate, citric acid, citric acid-based nano hydroxyapatite, cladribine, clarithromycin, clarithromycin ER, clavulanate, clavulanate potassium, Clenil Compositum, Clenilexx, Clindac-A, clindamycin, clindamycin phosphate, Clinisol, Clinolipid, Clino-san, Clinpro, ClinSupV3, ClinSupV3ER, clobetasol, clobetasol propionate, clobetasone butyrate, Clobex, Cloderm, clofarabine, clofazimine, Clomid, clomiphene, clomiphene citrate, clomiphene citrate HMG, clomiphene citrate rFSH, clonazepam, clonidine, clonidine HCL, clopidogrel, clopidogrel bisulphate, Clopirin, Cloratadd D, clorhexidine, cloricromen hydrochloride, clotrimazole, clotting factor FVIII, clozapine, CO2, co-amoxiclave, co-artemether, cocaine, cocaine HCl, Coccinia cordifolia, Coccinia grandis, Cocculine, cocoa, Co-codamol, codeine, codeine phosphate, codeine sulphate, codeine/paracetamol, coenzyme A, coenzyme Q, Coenzyme Q10, Cofact, Cofantrine, cogniVida, colchicine, Colchicum LM, Colchimax, colecalciferol, colesevelam, Colibiogen, colistin, Collagen tripeptide, colloids, Colonlytely, Colopeg, Colpermin, colposeptine, CombiflexOmega, CombiflexOmega peri, Committ Lozenge, Comp-01, Compleat, compound sodium alginate, Compound Sodium Lactate, Condrosulf, Conivaptan, Conjugated equine estrogen, conjugated equine estrogens, conjugated estrogen, conjugated estrogens, conjugated linoleic acid, Contovir, Contractubex, contusugene ladenovec, Coolprep, Cordyceps sinensis, Corever, Corsodyl, Cortef, corticosteroid, corticosteroids, corticotropin-releasing hormone, cortisol, CORvalen, CosmoFer, cotinine fumarate, cotrimoxazole, co-trimoxazole, coumarin/troxerutin, cranberry, Creapure, creatine monohydrate, Crescita, Cresnon, cromolyn, cromolyn sodium, Crosslinked hyaluronic acid, Cry j1-galactomannan, cryoprecipitate, crystallized human recombinant insulin, crystalloid, crystalloids, C-Tb, Curasept, curenext, Curosurf, cuscuta, custodiol, Custodiol HTK, cyanoacrylate, cyanocobalamin, cyclizine, cyclobenzaprine, Cyclokapron, Cyclomydril, cyclophosphamide, cycloserine, cyclosporine microemulsion, cyclo-Z, Cynara scolymus, cyproheptadine, cyproheptadine hydrochloride, cyproterone acetate, cysteine hydrochloride, Cystone, cytarabine, cytarabine, cytosine arabinoside, cytomel, D-(6,6-[2]H2)glucose, DA-9701, dabigatran etexilate, dabrafenib, DAC N-055, dacarbazine, daclatasvir, dacomitinib, dafalgan, Dafalgan codeine, Daflon, daiobotanpito, Dalap Duo, dalbavancin, d-alpha-tocopheryl acetate, daminaide 995, D-amphetamine, Danzen, DAOI-B, dapivirine, dapsone, daraprim, darexaban, darunavir, dasabuvir, dasatinib, Dawa-e-Musakkin, D-chiro-inositol, d-cycloserine, D-cyclosporine, dDAVP, debrisoquine, decitabine, Decortin H, deferasirox, deferoxamine mesylate, Deflux, Dehydrex, dehydroepiandrosterone, dehydroepiandrosterone sulphate, dehydroepiandrostrone, Dehypotin, delmopinol, delparantag, delta-9-tetrahydrocannabivarin, Demerol, Dentaid, deoxycholate, Depakote DR, Depalgos, Deplin, depot medroxyprogesterone acetate, deprenyl, dequalinium chloride, dermabrasion, Dermacid, Dermacyd, Dermacyd breeze, Dermacyd Femina, Dermacyd Femina Delicata, Dermacyd PH_DETINLYN, Dermacyd PH_Detinlyn Tangerine mix, Dermacyd Silver Floral, Dermacyd Silver Frutal, Dermacyd Tina Tangerina Mix, Dermacyn, Dermacyte, Dermatix, dermatop, Dermatophagoides pteronyssinus, Dermatophagoides pteronyssinus 1, Dermylex, Derris scandens Benth extracts, Desensebilize Nano P, Desensibilize KF, Desenssiv, desflurane, desipramine, desipramine hydrochloride, desloratadine, desmethyl-loperamide, Desmotabs, desogestrel, Desonide, desonide hydrogel, desorelle, Desoximetasone, Destin, desvenlafaxine, Detralex, dexamethasone, dexamethasone nanoparticles, dexamethasone sodium phosphate, dexamethasone USP Micronized, dexamethasone w, dexamphetamine, dexamphetamine SR, Dexcom G4 Platinum sensors, dexeryl, dexfenfluramine, dexketoprofen trometamol, dexmedetomedine, dexmedetomidine, dexotic, dexpanthenol, dextralink, dextran, dextran 70, dextrin, dextro-amphetamin, dextroamphetamine SR, dextromethorphan, dextromethorphan hydrobromide, dextropropoxifen, dextrose, D-galactose, dHACM, Dhatri Lauha Vati, Diabecinn, Diabetan, diacerein, diacetylmorphine, dialysate bicarbonate, dialysate calcium, Dialysate magnesium, dialysate sodium, Diamicron MR, Diamox, Dianeal, Diazepam, dichloroacetate, diclofenac, diclofenac diethylamine, diclofenac epolamine, Diclofenac HPBCD, diclofenac sodium, dicyclomine hydrochloride, didanosine, Didrogyl, Diena, Dietressa, digoxin, digoxine, dihydroartemisinin, dihydroartemisinin-piperaquine, diindolylmethane, Dilaudid, dill, diltiazem, diltiazem hydrochloride, dimercaptosuccinic acid, dimethicone, dimethindene maleate, dimethyl fumarate, dimeticone, dinaciclib, dinitrate isosorbide, dinitrochlorobenzene, dinoprostone, diosmectite, Dipeptiven, Diphenhydramine, diphenhydramine citrate, diphenhydramine hydrochloride, dipotassium-clorazepate, Diproderm, Diprospan, dipyridamole, dipyrone, diquafosol, diquafosol sodium, diquafosol tetrasodium, Disci/Rhus toxicodendron comp., Discogel, disodium cromoglycate, disodium cromoglycate/reproterol, disulfiram, diuretics, Diurisa, divalproate sodium, DL-alpha-tocopherol, d-Limonene, D-mannitol, D-methionine, dobutamine, docetaxel, doconexent ethyl ester, docosahexaenoic acid, docusate, docusate sodium, dofetilide, Dogmatil, Dolocam plus, dolutegravir, domperidone, domperidone maleate, donepezil hydrochloride, Dong Quai, Donifoxate, dopamine, dornase alpha, dorzolamide, dorzolamide hydrochloride, Dosin, doxapram, doxazosin, doxercalciferol, doxofylline, doxorubicin, doxorubicin hydrochloride, doxycycline, doxycycline hyclate, doxycycline monohydrate, doxycycyline, d-penicillamine, d-phenothrin mousse, DPP-4 inhibitor, D-reglis, Drioff, Drisdol, Drogenil, dronabinol, drospirenone, drotaverine hydrochloride, Drotin A, DRxHQ Brightning, Drysol, DTP3a-IPV-HBV/HIB vaccine, DTPw-HBV/HIB vaccine, Dual-time PET/CT, duloxetine, Duolans, DuoTrav APS, Duphastone, duramorph, DuraPrep, Durolane, Durolane SJ, dutasteride, dydrogesterone, Dydrogesterone M, Dynamiclear, Dynexan Mundgel, dypirone, E-004, eberconazole, EC-17, Echinacea, Echinacea purpurea, EchiTabPlus-ICP, Ecologic Femi, econazole nitrate, EconoPred Plus, ECP-019, Ecstasy, Ectoin, edaravone, edoxaban, edroxyprogesterone acetate, EDTA, EDTA-T, Eductyl, Efamax, efavirenz, EFB-0026, Effaclar, eflornithine, eformoterol, Efudex, EGb 761, EGb-761, Eicosapentaenoic acid, Ekzevowen derma, Elaeagnus angustifolia, elbasvir, Electrolytes, elemental calcium, elemental iron, Elestat, Eligen B12, Elimite, ellagic acid, Elo-Mel Isoton, eltrombopag, Emdogain, EMLA, empagliflozin, EMS acarbose, emtricitabine, En2nox, enalapril, Enalapril-TAD, encenicline, enclomifene citrate, endolubri, endotoxin, Engystol, Enjuvia, Enox, Enoxa, enoxaparin, enoxaparin sodium, Ensure, entecavir, enteric-coated mycophenolate sodium, Enterogermina, Enterolactis plus, Entonox, epalrestat, eperisone HCl SR, ephedra, ephedrine, ephedrine hydrochloride, Epimorph, epinastine hydrochloride, epinephrine, epirubicin, epithelium-off, EPO-alpha, epoetin-alpha, Epogam-1000, epoietin zeta, EPs 7630-solution, epsilon aminocaproic acid, EQ-5, equine estrogen, eremostachyslaciniata, Ergocalciferol, ergoferon, ergotamine, eribulin mesylate, Erigeron breviscapus, erlotinib, Erylik, erythomycin, Erythrina mulungu, erythritol, erythromycin, erythromycin ethylsuccinate, erythropoietin beta, Esarin, Escherichia coli endotoxin, Escherichia coli rHu GM-CSF, escitalopram, eslicarbazepine acetate, esomeprazole, esprico, Essiac, esterified estrogen, estetrol, Estrace, estradiol, estradiol acetate, estradiol hemimydrate, Estradiol valerate, estrodiol, Estrofem, EstroG-100, Estrogen, ethambutol, ethambutol hydrochloride, ethambutol+rifampin+pyrazinamide, ethanol, Ethinyl E2, ethinylestradiol, ethyl eicosapentaenoate, Ethyl vinyl acetate, Ethyl-eicosapentaenoic acid, ethylenediamine dihydrochloride in methylcellulose, ethylenediamine dihydrochloride in polyvinylpyrrolidone, ethylmethylhydroxypyridine succinate, ethynil estradiol, etidronic acid, etifoxine, etimicin sulphate, etinoline, etirinotecan pegol, etizolam CR, etodolac, etomidate, etonogestrel, etoposide phosphate, etoricoxib, E-TRANS Fentanyl hydrochloride, Eucerin, Euflexxa, Euminz, Eupasidin-s, Euphorbia prostrata, Euthyrox, Eutrophill, everolimus, Eviendep, eviprostat N, evofosfamide, EvoraKids, Evoser, EVT-301, exemestane, exendin, extended-release metformin, Extraneal, EYN-1601, EZ-Paque, EZ-Varibar, F-18 16-alpha-fluoroestradiol, F-18 Fludeoxyglucose, F-18 fluorodeoxyglucose, F-18 fluorothymidine, F-18 NaF, F-18-F-DOPA, F-18-fluoro-deoxi-glucose, famotidine, Fang(30), Fansidar, Faramir, Faringosept, Farlutal, faropenem, Fastact, fatty acid, F-Choline, F-deoxyglucose, FDG, FDG-(18F), FEC-100, felodipine, femanest, Femarelle, Femoston, Femulen, fenfluramine, fenofibrate, fenofibric acid, fenoterol, fenoverine, Fentanest, fentany, Fentanyl, fentanyl citrate, fenugreek seed, ferfolic, Ferogradumet, Feronia-XT, ferric carboxymaltose, Ferro Sanol, Ferro Zinc, FerroGrad, ferrosulphate, ferrous ascorbate, ferrous bisglycinate chelate, ferrous fumarate, ferrous glycine sulphate, ferrous glycine sulphate complex, ferrous iron sulphate, ferrous sulphate, FES, Fetal neural retinal tissue, fevipiprant, fexofenadine, fiacitabine, fibrinogen, fibrinogen concentrate, Ficortril, filgrastim, Filme Nasale, Filtek Silorane p90, Filtek Supreme—3M, Finalgon, finasteride, fingolimod, FiO2, firategrast, Fitostimoline, Flamazine, Flammacerium, Flammazine, Flavangenol, flax lignans, flecainide, flecainide acetate, Rector Tissugel Heparine, FloraGLO, Florajen3, florbetapir, Florestor, Florisia, florouracil, FloSeal matrix, FLT, flucinolone, fluconazole, fludarabine, Fludeoxyglucose, fludeoxyglucose F 18, fludiazine, fludrocortisone, fludrocortisone acetate, Fluimucil, Flumicil, fluocinolone, fluocinonide, Fluomizin, fluorescein sodium, Fluoride, fluorine F 18 fluorothymidine, fluorine F 18 sodium fluoride, Fluorine F 18-clevudine, fluorine F-18 fluorodopa, fluorine-18 fluorocholine, Fluorine-18 labeled L-leucine analog 1-amino-3-fluorocyclobutane-1-carboxylic acid, FluoroDeoxyGlucose, fluoro-L-thymidine, fluoro-L-thymidine-(18F), fluorometholone, fluorometholone, fluorothymidine, fluorouracil, fluoruracil, fluoxentine, fluoxetine, fluoxetine HCL, fluoxetine hydrochloride, flupentixol, fluphenazine decanoate, flupirtine maleate, flurbiprofen, flutamide, fluticasone, fluticasone furoate, fluticasone propionate, Folate, FOLFIRI, Folic acid, folinic acid, follicle stimulating hormone, Folrex, Forair, Foraseq, foretinib, Forlax, formalin, Formatris, Formoline L112, Formopen, formoterol, formoterol fumarate, formoterol Spiromax, formoterol/budesonide, formoterol/fluticasone, fosamprenavir, foscarnet sodium, foscavir, fosfolipovit, fosmidomycin, fostamatinib, Fostipur, Fragmin Protamin nano particle, framycetin sulphate, freezed-dried plasma, Fresofol, fructo-Oligosaccharides+Bacillus coagulans, Fructus Mume, FS VH SID 500 S-apr, FSH-r, Fucicort, Fucidin, fucoidan, fucoxanthin, fulvestrant, fumaric acid esters, Funhab, furosemide, Furoxone, fusidic acid, fusidic acid+adapalene, fuxiong san, Fu-zheng-qu-zhuo, FX80, G238, gabapentin, Gablofen, gadobenate dimeglumine, Gadolinium, gadopentetate dimeglumine, Gadopentetic Acid, gadoterate meglumine, gadoxetate disodium, galactooligosaccharide, galactose, Gallium-68 citrate, gallium-68-DOTATATE, galvanizator, Gambrosol-Trio, Gamma hydroxybutyric, gamma-amino-butyric acid, gamma-hydroxybutyric acid, gamma-linolenic acid, gamma-tocopherol, gammimmune N, ganciclovir, gancyclovir, Ganeden BC-30, Ganfort, ganirelix acetate, Ganoderma lucidum, gantacurium chloride, gargle dextromethorphan, Gargling, Garlicin, garsin, gastrofin, Gastrografin, Gastroview, Gastrus, gatifloxacin, Gatorade, Gavindo, GCJBP Laennec, G-CSF, G-CSF mobilized PB-MNC, gefitinib, Gel-200, gelatin, gelatin hydrogel, Gelclair, Gelesis-100, Gelofen, Gelofusine, Gel-One, Geloplasma, Geltim LP, gemcitabine, Gencydo, generic, GenF20 Plus, genistein, Genistein combined polysaccharide, gentamicin sulphate, gentamycin, gentamycine, Genteal HA, gestapuran, gestodene, ghrelin, GHRP-3, GIC KetacMolar Easymix, GIK, gimeracil, gin, Ginger, Gingko biloba, Ginkgo biloba, Ginkgolides Meglumine, Ginsana, Ginsana-115, Ginseng, Gintuit, Gipotef, glibenclamide, gliclazide, glimepiride, glimepiride ER, glipizide, GLP-1 analogs, glucagon, Glucagon-like peptide 2, Glucagon-like Peptide-1, Glucagon-like-peptide-1 amide, Glucantime, Glucerna, Glucion, glucomannan, glucopyrron, glucosamine, glucosamine sulphate, Glucose, glucose dependent insulinotropic polypeptide, glucose insulin potassium, Glucose potassium insulin, Glucose PROAMP 30%, glucose-bicarbonate-ringer lactate, glucose-dependent insulinotropic peptide, glucose-insulin-potassium, Glumin XR, glutamine, glyceral trinitrate, glycerin, glycerine, glycerine magnesium sulphate, glycerine trinitrate, glycerol, glycerol trinitrate, Glycerol/heparin, glyceryl nitrate, Glyceryl triacetate, glyceryl trierucate and glyceryl trioleate, glyceryl trinitrate, glycolic acid, glycopyrrolate, glycopyrronium, glycopyrronium bromide, glycosidase inhibitor, glycyrrhizin, GlyT-1 inhibitor-1, GlyTI-M, GM1 Ganglioside, GMP-grade HRV-39, GnRH antagonist, GnRHa, Gokshur-punarnava basti, Goksura, Golnaar, Golytely, Gonadotropin, gonyautoxin, goserelin acetate, GRALB, gramicidin, Grammidin, granisetron, Granulocyte colony-stimulating factor, granulocyte-macrophage colony-stimulating factor, grazoprevir, Grintuss, GSK-3532795, GTR, guaifenesin, Gualou, guanfacine, guanidinoacetic acid, guggul extract, guggulu, Gugulipid, Guifu dihuang pill, Guilongtongluofang, GutGard, GW-05, GW-2000-02, GWP-42004, Gymnema sylvestre, Gynazole 1, Gynomunal, Gynophilus, HAART regimen, Habitual hemostasis, Haelan, Haemate HS, Haemate P, HAIRAL 7, HalfLytely, halobetasol propionate, halometasone, haloperidol, haloperidole, haloperidole/perphenazine, Hamdoroid, Hametum, hangekobokuto, Haridra Khanda, Harpagophytum, Hartmann's solution, hawthorn, heavy silicone oil, Heberprot-P, Hedrin lotion, Hedrin Once, Heliox, Heliox gas, helium-hyperoxia, helium-powered albuterol, heme iron polypeptide, HemoHES 6%, Hemoionic, Hepalean, hepalean heparin, Heparin, heparin sodic, heparin sodium, heparin sulphate, HepaSphere, HepaSphere Microspheres, Hepavir, herba scutellaria Barbatae, Herbagut, Herbavate, Herbmed plus, heroin, HES 130/0.42, hesperidin, Hetastarch, hexacetonide triamcinolone, hexylresorcinol, hGM-CSF, Hibiscus sabdariffa, HiDHA, Hidroferol, High fibre, High flow oxygen, highly purified urinary follicle stimulating hormone, Hingwastaka choorna, histaglobulin, histamine, histidine-tryptophan-ketoglutarate Custodiol, HIVS-2004, HLA-A24-restricted peptide, HMG, hMG-IBSA, Hochu-ekki-tou, Holopon, homatropine, Homeodent, HP menotrophin, hPTH 1-34, HPV4 vaccine, hR3 MaB, hua tan san, hua tuo zai zao wan, Huangqi Bushen, Hugeltox, human acylated ghrelin, human chorionic gonadotrophin, human Chorionic gonadotropin, human FSH, human ghrelin, human menopausal gonadotropin, human recombinant epidermal growth factor, human recombinant erythropoietin, Human recombinant growth hormone, Human Recombinant Interferon-alpha, Human thrombin, Human urinary kallidinogenase, Humate-P, Hwangryunhaedoktang, Hyabak, Hya-Joint, HYAJOINT Plus, Hyalobarrier, hyaluronan, hyaluronan hydrogel, hyaluronate sodium, hyaluronic acid, hyaluronic acid-carboxymethylcellulose, hyaluronidase, Hyaneb, Hydrabak, hydralazine, hydralazine hydrochloride, hydrochloric acid oxybuprocaine, hydrochlorothiazide, hydrocizine, hydrocodone, hydrocodone/APAP, Hydrocortison, hydrocortisone, hydrocortisone acetate, Hydrocortisone hemisuccinate, hydrocortisone sodium acetate, hydrocortisone sodium succinate, Hydroeye, hydrofluoroalkane-134a beclomethasone dipropionate, Hydromorphone, hydromorphone hydrochloride, Hydromorphone Hydrochloride Extended-Release [HHER], hydroquinone, hydroxy camptothecin, hydroxy ethyl starch, Hydroxy Ethyl Starch 130/0.4, Hydroxy Ethyl Starch 70/0.55, hydroxycamptothecia, hydroxycarbamide, hydroxychloroquine, hydroxychloroquine sulphate, hydroxycitrate, Hydroxyethyl starch, Hydroxyethyl starch 130/0.4, hydroxyethyl starch+sodium chloride, hydroxyethylsterke, hydroxyisohexyl 3-cyclohexene carboxaldehyde, hydroxynortriptyline, hydroxyprogesterone caproate, hydroxypropylmethylcellulose, Hydroxyurea, hydroxyzine, Hylagan, Hylenex, Hylo-Comod, HYNR-CS inj, hyoscine, hyoscine bromide, hyoscine butylbromide, hyoscine hydrobromide, hyoscine N-butylbromide, hyoscine-N-butyl bromide, hyoscine-N-butylbromide, hyosine butylbromide, hyperbaric bupivacaine, hyperbaric levobupivacaine, hyperbaric oxygen, Hyperbaric prilocaine, HyperHAES, hypericum, Hypericum extract WS-5570, Hypericum perforatum, Hyperimmune intravenous immunoglobulin, hyperosmolar sodium lactate, Hyperoxia, HyperSal, Hypersaline, Hypertonic lactate, Hypertonic saline, hypertonic saline/Dextran-70, hypertonic saline/hetastarch, hypertonic saline-dextran, Hypiran, Hypocol, Hypo-osmolar ORS, hypo-osmolar riboflavin, hypotonic saline, hypromellose, Hyruan Plus, I-123 15-(4-iodophenyl)-3(R,S)-methylpentadecanoic acid, I-123-ADAM, I-124, I-125, I-131, Ibalgin, ibandronic acid, ibopamine hydrochloride, Ibuprofen, ibuprofen arginine, ibuprofen sodium, ibuprofen/acetaminophen, ibuprofen/phenylephrine, ibuprofen/phenylephrine/chlorpheniramine, ibuprofenamine hydrochloride, Icaps, Ichthraletten, iclaprim, icodextrin, icodextrin dialysate, icosapent ethyl, icosapent ethyl ester, ICT-107, idarubicin, idebenone, idraparinux sodium, IFN, IFN-beta-1, IFN-gamma, Ifosfamide, IK-01, IK-5001, ikshvadi avaleha, IL-1Ra, IL-2, iloperidone, Iloprost, imatinib, imatinib mesylate, imatinib-TL, Imazol, imipenem, imipramine, Imiquimod, immediate release methylphenidate, immediate-release oxcarbazepine, IMMUNATE S/D, Immunine, immunoglobulin, immunoglobulin G, ImmunoHBs, immunomodulator drug, IMMUVAC, Imodium Plus, Imotun capsule, Imovane, Imprimis, Imuran, Imurril, inactivated poliovirus, indacaterol, indacaterol maleate, indapamide, indisetron, Indium 111-DTPA, indium In 111 folic acid, Indium In 111 pentetreotide, Indivina, indobufen, indocyanine green, indole-3-carbinol, Indoleamine 2,3-dioxygenase peptide vaccine, indomethacin, indoximod, Ineo, Inersan, Infloran, Influcid, Infufer, Ingavirin, ingenol, ingenol disoxate, Ingenol Mebutate, Innolak, INOmax, inositol, IN-PHARM-002, inspired oxygen, Instillagel, insulin, insulin aspart protamine+insulin aspart, Insulin Nph, IN-SUPR-002, interferon, interferon alpha-2b, interferon alpha 2a, interferon alpha-2, Interleukin-2, Intralipid/heparin, Intralipid/Liposyn, intralipids, IODEX, Iodex Ultragel, Iodide, Iodine, iodinel 125, iodine-131, iodopovidone, Iodosorb, iohexol, ionic silver, Iopamidol, Iota-Carrageenan, IP-007, Ipocal, ipraglifrozin, ipramol Ster-Neb, ipratropium, ipratropium bromide, ipratropium bromide HFA-134a, ipratropium bromide/salbutamol sulphate HFA, IPT-sulphadoxinepyrimethamine, iralvex, Iranian clozapine, irbesartan, Irgacare MP, iridium-192, irinotecan, irinotecan sucrosofate, Iron, iron bisglycine chelate, iron Carboxymaltose, iron hydroxide polymaltose, iron isomaltoside 1000, iron sucrose, iron sulphate, Iron supplement, iron-HES, IRONPLUS, isavuconazonium chloride/sulphate, Ismigen, Isocolan, isoflavone, isoflavones, isoflurane, Isoniazid, isoprinosine, isopropyl alcohol, Isopto-Dex, isosorbide-dinitrate, isosulfan blue, Ispaghula husk, istradefylline, itraconazole, ivabradine hydrochloride, ivacaftor, IVF-M HP, ixabepilone, Ixeris of sonchifolia Hance, Jasmine officinale, Jiangtang Xiaozhi, Jobelyn, Jonosteril, josamycin propionate, Juzentaihoto, Juzen-taiho-to, kalcipos-D, Kalinox, Kallunk oxide, kanamycin, kanamycin/metronidazole, kandhaga rasayanam, Kanuka, Kardostin, Kava, Kefir, Keflin, keishi-bukuryo-gan-ka-yokuinin, kelocote, Kemstro, kenacort, Kenalog, Kenalog-40, ketamine, ketamine hydrochloride, Ketasyn, ketoconazole, ketofol, Ketoprofen, ketoprofen lysinate, ketoprofen lysine, ketorolac, Ketosteril, ketotifen, ketotifen fumarate, KHCO3, Kikyo-to, Kligman's Trio, Klimadynon, Klorhex, Klyx, koloforta, Konakion MM, kopyrrol, KP24, KRG (Korea Red ginseng), Ksenaltena, Kuan-Sin-Yin, Kukkkutanda Twak, KULIST, kunxian, kunzea oil, Kytta-Salbe f, KyungBangn Gamisoyosan, KyungBangn-Gamisoyosan-x-gwarip, L bupivacaine, L sakei KCTC 10755BP, L-[1-C]-valine, LAAM, labetalol, labetalol hydrochloride, L-acetyl carnitine, L-acetylcarnitine, Lacidophil, lacosamide, Lacribell, lactase, lactate, lactate Zhry capsules, Lactated Ringer's solution, Lacteol, LacTEST, lactic acid, lactitol monohydrate, Lactobacillus acidophilus, Lactobacillus acidophilus L-92, Lactobacillus acidophilus NCFM, Lactobacillus brevis CD2 lozenges, Lactobacillus casei strain Shirota, Lactobacillus fermentum, Lactobacillus GG, Lactobacillus paracasei Lpc-37, Lactobacillus reuteri, Lactobacillus reuteri RC-14, Lactobacillus rhamnosus, Lactobacillus rhamnosus GG, Lactobacillus rhamnosus GR-1, Lactobacillus salivarius LS01, Lactobacillus strain Th1, Lactobacillus reuteri DSM 17938, lactoferrin, lactofiltrum, Lactol, lactose, lacto-tripeptide Ile-Pro-Pro (IPP), Lactowell, Lactulose, L-alanine, L-Alpha Glycerylphosphorylcholine, Lamaline, Lambdalina, lamivudine, lamotrigine, lansoprazole, lanthanum carbonate, lanzoprazole, lapatinib, Lapis judaicus, L-arginine, L-arginine aspartate, L-arginine hydrochloride, laropiprant, L-ascorbic acid, latanoprost, latanoprost PK, Lattoglobina, Lavasept, Lavender, Laxabon, Lax-asab, Laxymig, L-baclofen, LBSA-0103, L-citrulline, Lcr Regenerans, L-dopa, Ledermix, ledipasvir, lenalidomide, Lepisor, leptin, leptin A, leptin A-100, lercanidipine, lesinurad, Lessina, letibotulinumtoxinA, letrosol, letrozole, Leucine, leucovorin, leucovorin calcium, Leukichtan, leukotriene D4, leuprolide, leuprorelin acetate, levacetylmethadol, levalbuterol, levalbuterol hydrochloride, levamisole, levenorgesterol, levetiracetam, levobupivacaine, levocabastine hydrochloride, levocarnitine, levocetirizine, levodopa, Levodyn, levofloxacin, levomefolate calcium, levomepromazine, levonorgestrel, levophed, Levosert-20, levosimendan, levothyroxin, levothyroxin sodium, Levothyroxine, levothyroxine sodium, L-glutamine, L-GSH, lianhua qingwen capsule, Lianhuaqingwen, LIBERTAL, Licaps, Licefreee, LiceMD, licorice, lidocaine, lidocaine hydrochloride, lidocaine/adrenalin, lidocaine/tetracaine, LidoDyn, lignocaine, linaclotide, linagliptin, Lingzhi, Linolacort Hydro, linoleic acid, LinumLife EXTRA, Lioresal, liothyronine, liothyronine sodium, liothyronine sodium/triiodothyronine, Lipicure80, Lipidem, lipid-nutrients, Lipigesic M, Lipilou, Lipinon, Lipiocis, Lipiodol, LipoCol Forte, Lipofundin MCT, Lipogel, lipoic acid, Lipophilic hydroxy acid, Lipoplus, lipopolysaccaride, Lipopolysaccharide, Liposic, liposomal 9-Nitro-20, cyclosporine, liposomal amphotericin B, liposomal bupivacaine, liposomal doxorubicin hydrochloride, liposomal iron, liposomal latanoprost, liposomal prednisolone, Lipuro, LiQ-Nol, liquid nitrogen, liquor carbonis distillate, lisdexamfetamine, L-isoleucine, Lisonorm, listerine, Litesse Ultra, lithium, lithium carbonate, Lithium gluconate, lithium sulphate, Litramine, liu wei di huang pill, Livact, Livact Granules, L-leucyl-L-leucine methyl ester, (lidocaine hydrochloride, L-lysine, LMB B, LMB C, L-Menthol, L-methylfolate, LMX-4, L-NAME, L-NG-monomethylarginine, locapred, Locobase Repair, lofexidine, lomerizine, Lomotil, lonafarnib, Lo-Ovral, loperamide, lopinavir, loratadine, lorazepam, lorcaserin, lornoxicam, lornoxicam SR, lornoxicam/paracetamol, Lortab, losartan, loteprednol, loteprednol etabonate, Louxiangdan Tongxin, lovastatin, loversol, Low dialysate calcium, low-dose fentanyl, loxoprofen sodium, lozenges, LPS, L-selenomethionine, I-tetrahydropalmatine, L-Theanine, L-thryoxine, L-thyroxin, L-Thyroxine, L-Tryptophan, L-tyrosine, lubiprostone, Lubricin, lucinactant, Lugol's iodine, lumacaftor, Lumartem, lumefantrine, lupeol, Luphere, lurasidone, luseogliflozin hydrate, Lutrepulse, LY-3053102, Lyc-o-Mato, lycopene, lymecycline, Lymphdiaral Basistropfen, Lymphomyosot, Lyral, lysergic acid diethylamide, lysine acetylsalicylate, lysozyme, lysozyme hydrochloride, LZ Complex3, MAb 17-1A, Mablet, macadamia nut/oil, macrogol, macrogo13350, Madhumega chooranam, magnesia, Magnesium, magnesium bicarbonate, magnesium carbonate, magnesium chloride, magnesium citrate, magnesium hydroxide, magnesium oxide, magnesium pidolate, magnesium sulphate heptahydrate, magnesium sulphate, magnesium-aluminium antacid, MagniXene, Magnox 520, Malacur, malic acid, maltodexterine, Maltodextrin, Mannan Binding Lectin, mannitol, maralixibat chloride, Marijuana, Marine phospholipids, Maxepa, Maxidex, Maxigesic, Maxigesic 325, Maximum D3, Maxitrol, Maxolon, Maxomat, mayinglong musk, MaZiRenWan, mCPP, MDMA, mebendazole, mebeverine, MEBO, Mebo, mecobalamin, mecobalamin monohydrate, MediHoney, medroxyprogesterone, mefenamic acid, mefloquine, Megace, megesterol, megestrol, megestrol acetate, meglumine antimoniate, meglumine antimony, melatonin, melatonin SR, Melfade, Melissa officinalis, melissan, melitracen, meloxicam, melphalan, memantine hydrochloride, menadiol phosphate, menaquinone 7, menatetrenone, Menest, Menevit, Menopur, menotrophin, menthol, menthol nicotine polacrilex, MEOPA, Mepentol Leche, meperidine, Mepiform, Mepilex Ag, mepivacaine, mercaptopurine, Merional, Merislon, Merlin, Meropem, meropenem, Mersina, mesalamine, Mesalazine, mesalazine EC, mesna, Meso Tetra Porphine, meta-chlorophenylpiperazine, metaclopramide, Metamin 3D, metamizol, metamizole, metamizole sodium, metaraminol, metclopramide, Meteospasmyl, metformin, mefformin ER, metformin hydrochloride, metformin IR, mefformin SR, metformin/glimepiride, metformin/sitagliptin, methacholine, methacholine chloride, methadone, methadyl acetate, methamphetamine, Methergine, methimazole, methionine, methocarbamol, methotrexate, methoxsalen, Methyl 5-aminolaevulinic Acid, methyl aminolevulinate, Methyl B12, methyl nicotinate, methylcellulose, methylcobalamin, methylene blue, methylene blue dye, methylphenidate, methylphenidate extended release, methylphenidate hydrochloride, methylprednisolone, methylprednisolone acetate, methylprednisone, metoclopramide, metoprolol, metoprolol succinate ER, metoprolol tartrate, metreleptin, metrifonate, MetroGel, metronidazole, metronidazole benzoate, metypredsolone, mFOLFOX6, MgSO(4), micafungin, miconazole, miconazole nitrate, microcrystalline cellulose, micronized progesterone, Microvlar, midazolam, midazolam hydrochloride, midodrine, mifepristone, Mignar MF, Migraleve, Miniderm, Minidril, MINIRIN Oral Lyophilisate, minocycline hydrochloride, minocycline-EDTA, minoxidil, mirabegron, MiraLax, mirazid, mirogabalin, mirtazapine, misoprostol, miswak, mithramycin, mitiglinide, mitomycin, mitoxantrone, MK-0507a, MLF-541, MMR vaccine, MNC-01, modafinil, molindone hydrochloride, Momendol, mometasone, mometasone furoate, mometazon froat, Momordica charantia, monoammonium glycyrrhizinate glycine DL-methionine, monocaprin, monochloroacetic acid, monosodium glutamate, Monovisc, montelukast, montelukast sodium, Monurelle Biogel, Moov, Morphine, morphine sulphate, Motherwort extract, Motilium, Motore, Motrin, Movicol, movicolon, moxaverine, moxifloxicin hydrochloride, MPT64, MS Contin, Mscontin, Mucinex, Mucinex D, Mucoangin, Mucopolysaccharide polysulphate, mudga yoosha, MultiBic, Multivitamin, mupirocin, murepavadin, MuStD05 vaccine, Mutaflor, Muxan, mycophenolate mofetil, mycophenolic acid, Mydriasert, myfortic acid, Mygliol 810, myo-inositol, Myrrhinil-Intest, mythylprednisolone, N acetyl-aspartyl-glutamic acid, N(2)-L-alanine L-glutamine dipeptide, N(G)-monomethyl-L-arginine, N-13-ammonia, N2O—O2 mixture, n-3 fatty acid, n-3 PUFA, n-3 PUFAs, Na ascorbate, Na sulphate, Na134I, Nabi-HB, nabiximols, N-acetyl-aspartyl-glutamate, N-acetylcysteine, N-acetylsucteine, NaCl, NaCl/hydroxyethylstarch, nadroparin calcium, NaF, Naftin, nalbuphine hydrochloride, naloxone, naltexone, naltrexone, naltrexone hydrochloride, naltrexone implant, naltrexone SR, naltrexone-HCl, nandrolone decanoate, NanoCis, nanocrystalline silver, Naoan, naphazoline, naphthoquine, Naprosyn, Naproxen, naproxen sodium, naproxene, naratriptan, Nardostachys jatamansi, Nartana, Nasaleze, Nasha Dx, Nasik, Nasopore, native glp-1, natriumchloride, natriuretic peptides, Natsa, Nattokinase, natural progesterone, Natural source d-alpha-tocopheryl acetate, Naturetti, nebivolol, nefopam, NeoGep, neomycin, neomycin sulphate, NEOSORB PLUS, neostigmine, Neptazane, nesdonal, Nettle, Neumoterol, Neumotex, Neuragen, Neuraxen, Neurodoron, neuropeptide Y, Neuroskin forte, Neurotec, NeutraLice, nevirapine, Newfill, Nexrutine, NG-monomethyl-L-arginine, NH(3), niacinamide, Niacin-ER, Niacor, nicardipine chlorhydrate, nickel sulphate, nicoboxil, Nicoderm, nicorandil, nicotinamide, nicotine, nicotinic acid, Nidadd, nifedipine, Niferidil, Nigella, Nigella sativa, nilotinib, nimodipine, nintedanib, nitrate, nitrates, nitric oxide, Nitrite, Nitrofurantoin monohydrate, nitrogen, nitroglycerin, nitroglycerine, Nitronox, nitroprusside, nitrous oxide, NKP-5191, N-methyl glycine, N-nitro L-arginine-methylester, Noex, nomegestrol acetate, Nonafact, nonivamide, noradrenalin, noradrenaline, Noragol, norepinephrine, norethindrone acetate, norethisterone, norethisterone acetate, Norethisterone enantate, norgestimate, norgestrel, Normia, Normoxia, nortriptyline, Norzyme, Notuss, Novafen, Novelose, Nozinan, nucleoside/nucleotide analogue, NuLYTELY, Nuon, Nutrineal, NYC-0462, Nycoplus, nystatin, Nystatin LF, O2, O3mega, O3mega+ Joy, Oasys, Oaxaca, octafluoropropane, octanediol, octanoic acid, Octaplas, OctaplasLG, octenidine, Octenidine dihydrochloride, Octenidol, Octenisept, octylcyanoacrylate, Oestrofeminal, olanzapine, Olea, Oliclinomel N4, oligofructose-enriched inulin, oligomeric lactic acid, olmesartan, olmesartan medoxomil, olodaterol, olopatadine hydrochloride, ombitasvir, omega-3, omega-3 ethyl-eicosapentaenoic acid, omega-3 fatty acid, omega-3 polyunsaturated fatty acid, Omegaven, Omegaven Fresenius, omeprazole, omeprazole and bicarbonate, omidenepag isopropyl, Omni-stat, ondansetron, OPF-102, Opioid, Opti-EPA, Optifree RepleniSH, Optiginon, Optiginon vulvovaginal candidiasis, Opti-mist, Optivate, Optive, Optive Fusion, Optive Sensitive, OPV, Oramorph, Oraqix, OraTest, Orexigen, Origanum vulgare, Orinase, Orlaam, orlistat, Orlistat Canon, ortho-phthaldehyde, Orthokine, Oscillococcinum, oseltamivir, Osmitrol, Ostend, Ostenil plus, osteoclastogenesis inhibitory factor (OCIF), Ostine, Ostoforte, Osvix, otamixaban, oteracil potassium, Otociriax, Otodolor, otrivin, Ovestin, oxaceprol, oxaliplatin, oxantel pamoate, oxantel-albendazole, oxazepam, oxcarbazepine, oxitropium, oxybutynin, oxybutynin IR, oxybutynin ratiopharm, oxycdone, oxycodone, oxycodone CR, oxycodone HCl XL, oxycodone IR, oxycodone/naloxone, Oxygen, oxymetazoline HCl, oxymix, OxyNeo, oxyntomodulin, oxytocin, ozone, Paclitaxel, paclitaxel liposome capsulated, paclitaxel-PM, palanosetron hydrochloride, palifosfamide, paliperidone, paliperidone palmitate, palladium Pd 103, palmitoylethanolamide, Paludrine, pamidronate disodium, p-aminosalicylic acid, Panadol, Panax ginseng, Panax notoginseng saponins, panchtiktaguggulu ghrita, Pandel, Pandughni vati, Pangramin, pangramin SLIT HDM-mix, Panhematin, pantazocine, Panthenol, Pantoline, pantoprazole, pantoprazole sodium sesquihydrate, pantoprazole-magnesium, Panzytrat, papain, papaverine, Paracetamol, paracetmol, paraffin, parasikayavanyadi, Parcel, paricalcitol, paritaprevir, paromomycin sulphate, paroxetine, paroxetine hydrochloride, paroxetine hydrochloride controlled release, paroxetine hydrochloride hemihydrate, paroxetine mesylate, Pascoflair, Paser, Pavulon, pazopanib, Pd-103, PDS PLUS, PDsol 12, Pectin, Pedialyte, PediaSure, Pediatril, PedyPhar, PEG, PEG-3350, PEG-4000, PEG-CS, PEG-IL-2, peginterferon alpha-2a, peg-interferon alpha, Pegorion, pegylated interferon, pegylated interferon alpha, pegylated liposomal doxorubicin hydrochloride, pemetrexed, pemetrexed disodium, penicillin, penicillin VK, penicilline, Pennel, Pentacarinat, Pentaerythrithyltetrantrate, Pentaglobin, Pentalong, Pentasa, Pentaspan, pentavalent antimony, Pentoxifylline, pentoxyphylline, Peplin, Peptamen 1.5, peptide YY, peramivir, perampanel, Perenterol Forte, perfluorocarbon-exposed sonicated dextrose albumin, perfluropropane, perflutren lipid microsphere, Perforan, pergolid, Peridex, perifosine, perindopril, Perio-aid, Periogen, periorinse, Permacol, permethrin, Permixon, perphenazine, Persantine, Persica, petasine, pethidine, pethidine hydrochloride, Peucedanum japonicum Thunb, Peya, pH altered Tc-99m sulfur colloid, phenazopyridine hydrochloride, phenelzine sulphate, Phenergan, phenobarbitone, Phenolphthalein, phenoxymethylpenicillin, Phenprocoumon, phenserine tartrate, phentermine, phentermine/fenfluramine, phentoloamine, phenylacetate, Phenylephrine, Phenylephrine hydrochloride, phenylephrine hydrochloride extended release, phenyramidol, phenytoin, PhosLo, phosphatidylcholine, phosphatidylcholine-associated acetylsalicylic acid, phosphatidylserine, phosphorus, phosphorus-188Re, Photocil, Photrexa ZD, phyllanthus, phyllanthus urinaria, phylloquinone, PhyllPro, Physioneal, Physioneal 35, phytoestrogen, phytonadione, phytosphingosine, phytosterols, Piascledine, Pidogrel, pimecrolimus, pimonidazole hydrochloride, pinaverium bromide, Pindolol, pioglitazone, piperacillin, piperacillin-tazobactam, piperaquine, Pippalyadi taila, pirarubicin, pirfenidone with modified oxide diallyl disulfide, piritramid, Piritramide, piroxicam, Pistacia mutica extract, pisuvastatin, Pitocin, pituitary adenylate cyclase-activating polypeptide, pivmecillinam hydrochloride, pivmecillinum, Plantago extract, Plantago ovata, plantago ovata husk, Plasbumin 5, Plasmalyte, Plasmalyte 148, Plasma-Lyte 148, plasmalyte A, Plasma-Lyte A, Plasmalyte-A, plasmin, Platinol, Platosin, PLGA, PLP-10, PluroGel N, Plurogel PN, Pluronic F-68, PluronicF-127, PN-2034, podophyllin, policosanol, polihexanide, poly(lactide-co-glycolide), polydatin, polydeoxyribonucleotide, polydioxanone, polyethylene glycol, polyethylene glycol 3350, polyethylene glycol 3350 laxative, polyethylene glycol 400/propylene glycol, polyethyleneglycol3350, polyferose, polyglucosamine L-112, polyglycolic acid, Polygonum cuspidatum extract, polyhematoporphyrin, polyhexamethylene biguanide, polyhexanide, poly-L-lactic acid, polymaltose, polymeric nanoparticle docetaxel, polymyxin E, polysaccharide-K, polysporin, polystyrene sulfonate, polyunsaturated fatty acids, polyvinylpyrrolidone, pomalidomide, POMx, poractant alpha, porcine secretin, porfimer sodium, Portia, Portulaca oleracea, Posiformin, Posterisan, Potassium, potassium bicarbonate, potassium canrenoate, Potassium chloride, potassium citrate, potassium clavulanate, potassium dichromate, potassium hydroxide, potassium iodide, potassium iodine, potassium magnesium citrate, potassium nitrate, Potassium oxalate, potassium phosphate, potassium sodium citrate, povidone, povidone iodine, povidone K-25, PPC heparin, PPSB-SD, PPSV23, pralidoxime, pramipexole, pranoprofen, prasugrel, pratimarsha nasya, pravastatin, praziquantel, prazosin, Pred Forte, PredKeterolac, prednesolone, prednisolone, prednisolone acetate, prednisolone sodium phosphate, prednisone, Prednisone Axapharm, pregabalin, pregnacare plus, pregnenolone, premixed nitrous oxide, Premixed nitrous oxide and oxygen, Prempro, Presillion Plus, Prevadh, Prevenox, PRGF, pridopidine, prilocaine, primaquine, primatene, Prismocitrate 10/2, PRK-124, ProAlgaZyme, probenecid, Probiotic lactobacilli reuteri, probiotic preparation bifilac-beals, Probiotic pur, Probiotics-Bio-plus, procaine, Procalcitonin, procarbazine, procarbazine hydrochloride, procaterol, procaterol hydrochloride, Prodarsan, Proferrin, Proferrin ES, progesterone, progestin, progestin medroxyprogesterone acetate, progestins, proguanil, progynova, promethazine, PROMETRIUM, Promiseb, Pronase, Prontoderm, Prontogest, Prontosan, propacetamol, propafenone, propanol-2-ol, proparacaine, proparacaine hydrochloride, propatylnitrate, Propess, Propofol, Propofol-Lipuro, propolis, Propoven, propoxyphene napsylate, propranolol, propranolol LA, propyl nicotinate, propylthiouracil, propylthiouracile, prostaglandin F2alpha, ProstateEZE Max, Prostin E2, prosultiamine, Pro-Symbioflor, protamine sulphate, ProTectis, Protein C zymogen, Prothromplex Total, protionamide, Provera, Provocholine, prucalopride, Pseudoephedrine, pseudoephedrine hydrochloride, psilocybin, Psoralen, Pulmonarom, pumactant, Punarnavadi Mandur, Punica granatum extract, Purethal Grass, Purite, PVP-iodine, Pycnogenol, Pycrinil, Pylorex plus, pyrazinamide, Pyridium, pyridostigmine, pyridoxal, pyridoxine, pyridoxine hydrochloride, pyrimethamine, pyrimethamine/sulfdoxine, pyrimethamine/sulphadoxine, pyronaridine, QAV-680, Qideng Mingmu, Qing-shang-fang-feng-tang, Quadriderme, quetiapine, quetiapine fumarate, quetiapine IR, quinapril/hydrochlorothiazide, quinidine, quinidine sulphate, quinine, quinine sulphate, Qurs Mufasil, Qurse mafasil, R-610, rabeprazole, rabeprazole sodium, racementhol, racemic adrenaline, racemic albuterol, racemic bupivacaine, radio-active iodine, radioiodine, radium Ra 223 dichloride, raktashali odana, raloxifene, raltegravir, ramipril, ranitidine, rapifen, Read-Cat 2, Realsil, rebamipide, reboxetine, Recaldent, recoflavone, recombinant alpha-interferon, recombinant asparaginase, recombinant asparginase, recombinant brain natriuretic peptide, recombinant factor Vlla, Recombinant factor VIII, recombinant flt3 ligand, Recombinant Follicle Stimulating Hormone, Recombinant Follicular Stimulating Hormone, recombinant FSH, recombinant FVIIa, recombinant gonadotropin, recombinant growth hormone, recombinant hepatitis B vaccine, recombinant human activated protein C, recombinant human coagulation factor VIII, Recombinant human epidermal growth factor, recombinant human erythropoietin, recombinant human growth hormone, recombinant human hyaluronidase, recombinant human insulin-like growth factor I, recombinant human interleukin-10, recombinant human LFA3, recombinant human luteinizing hormone, recombinant human prolactin, recombinant human tissue plasminogen activator, recombinant interferon alpha, recombinant interferon alpha-1b, recombinant interferon alpha-2a, Recombinant interferon beta, recombinant interferon gamma, recombinant interleukin-12, recombinant interleukin-6, recombinant methionyl human leptin, recombinant tissue plasminogen activator, recombinant tissue plasminogen activator (rt-PA), recombinant tumor necrosis factor, Recombinant VZV glycoprotein gE antigen, Recombinant-methionyl human stem cell factor, Recuperat-ion, red clover phytoestrogens, redback spider antivenom, Reduced glutathione, regorafenib, ReliDerm DT, Remegal, Remember-fX, remifentanil, Rengalina, repaglinide, Replavite, Replenine, Replenine-VF, rEPO, reproterol, Rescueflow, Resfenol, resiniferatoxin, Respocort, Restylane, resveratrol, resveravine, Resvida, Retacnyl, Retaine, retapamulin, Retaron, retinol, retinyl palmitate, Reumatocept, revaprazan, Revia, rFSH, r-FSH, Rg3, rhenium-188 hydroxyethylidene diphosphonate, Rhodiola Crenulata, Rhodiola rosea extract, rhTSH, rHuEpo, r-HuEPO, Rhus coriaria powder, ribavirin, riboflavin, ribose, Ricrolin TE, Rifafour, rifambutin, rifampin, rigosertib sodium, Rikkunshito, Rikuzit, rilapladib, rilpivirine hydrochloride, rimostil, Ringer-Acetat, Ringer-lactate/dextrose, Ringer's lactate, ripasudil hydrochloride hydrate, risedronate sodium, risperidone, ritonavir, rituximab, rivaroxaban, rivastigmine, r-metHuGDNF, RMJH-111b, Rocuronium, roflumilast, Rohto Hydra, ropinirole, ropivacaine, Rosa damascena, rosiglitazone, rosiglitazone maleate, rosuvastatin, rotavirus vaccine, rotigotine, Rowachol, Rowasa, RRR-alpha-tocopherol, R-sibutramine, Rubus occidentalis extract, rucoronium, ruzasvir, S(−)-bupivacaine, Saccharomyces Cerevisiae CNCM I-3856, saccharose, S-Adenosyl-L-Methionine, S-adenosyl-L-methionine disulphate p-toluene-sulfonate, S-adenosyl-methionine, saffron, Safoof-e-Muhazzil, SagaPro, sage, sagopilone, Saireito, salacinol, salbutamol, salbutamol sulphate, salbutamol sulphate HFA, salicylate, salicylic acid, Saline, saliva natura, Salofalk, salsalate, Salvia officinalis, salvianolate, salvinorin A, Samital, S-amlodipine besylate, sandalwood oil, Sandostatina LAR, Sanjiu weitai granules, Santafer, sapropterin dihydrochloride, saquinavir, sarcosine, saxagliptin, Scandicain, secretin human, Sedron, segesterone acetate, selegiline, Selenase, Selenium, selenium yeast, selenomethionine, Selenplus, SELG 1000, selinexor, semaxanib, Senna, SennaS, sennoside, sennosides, Sensodyne dentifrice, Sense, Septanazal, Septilin, Septolete total, Seraseal, Serenoa repens, Seresis, sericin, Serkangabin, Seroflo, Seromycin, SeroVital, sertraline, Setarud, S-etodolac, sevelamer carbonate, sevelamer hydrochloride, sevoflurane, SGPF-C, ShanStar, Shebet oil, Shegan, Shenwu, shexiang baoxin, Shi-Bi-Lin, Shinbaro, Shirazi Thymus Vulgaris, short chain fatty acid, Shuxuening, sibutramine, Siccafluid, sildenafil citrate, Silexan, stin, silicon oil dimeticone, silicone, silodosin, silver, Silver diamine fluoride, Silver diammine fluoride, Silver fluoride, silver nanoparticles, silver sulfadiazine, silver sulphadiazine, silver zinc sulfadiazine, silybin-phytosome, silybin-vitamin E-phospholipids, Silybum marianum, Silymarin, simethicone, simvastatin, Sinecort, Sinucare, Sinuclean, Sinutab, sirolimus, sisomicin, sitagliptin, Skoal, SLITone ULTRA, Sb-Niacin, SM-1, Smecta, SmellX, S-methyl-L-thiocitrulline, SMOF Kabiven, SMOF lipid, SmofKabiven, SMOFlipid, SNUBH-NM-333, sodium, sodium acetate C11, sodium alendronate, sodium alginate, sodium bicarbonate, sodium bituminosulfonate, sodium butyrate, sodium carboxymethylcellulose, sodium chenodeoxycholate, sodium chlorate, Sodium chloride, sodium chlorite, sodium chondroitin sulphate, sodium citrate, sodium cromoglycate, sodium dialysate, sodium ferrous citrate, Sodium Fluoride, sodium fluoride 18-F, sodium fusidate, Sodium Fusidate, sodium hyaluronate, sodium hydrogen carbonate, sodium hypochlorite, sodium iodide, sodium lactate, sodium L-ascorbyl-2-phosphate, Sodium lauryl sulphate, sodium monofluorophosphate, sodium nitrate, Sodium nitroprusside, sodium octanoate, sodium oxybate, Sodium Phenylbutyrate, sodium phosphate, sodium picosulphate, sodium picosulphate and magnesium citrate, sodium selenite, sodium stibogluconate, sodium thiopental, sodium tungstate, sodium valproate, sodium zirconium cyclosilicate, Sodium-Nitroprusside, sodium-selenite, Sofinox, Sofinox rd, sofosbuvir, Sojourn, Sokatin, solidago, solifenacin, Solosite, Soluble epoxide hydrolase, Solu-Cortef, somatostatin, Songha Night, sorafenib, sorbitol, sotalol, Soventol HydroCort, soy isoflavones, soya phytoestrogens, Spersadex, sphingomyelin, spironolactone, Spirulina, Spirulina platensis, Spongel, SQIN, squill extract, SSRIs, stachys lavandulifolia, Staloral, Staloral 300, Staloral 300R, standard doxazosin, standard silicone oil (PDMS), stannous compounds, stannous fluoride, statin, Statins, stavudine, StemEnhance, StemFlo, Steroid, steroid-dexamethasone, steroids, Stimdate, Strefen, strepsils, Strepsils Original, streptomycin, streptomycin sulphate, strontium chloride, Structum, Suan Tzao Ren Tang, Suan-Zao-Ren Tang, SUBA-itraconazole, Subetta, Subgam, succinylcholine, Suc-HSA (succinylated human serum albumin), sucralphate, sucralose, Sucromalt, sucrose, sudachi peel, Sudafed, sufentanil, Sugammadex, sulfadoxine, Sulfadoxine pyrimethamine, sulfadoxine/pyrimethamine, sulfamethoxypyrazine, sulfasalazine, sulfentanil, sulfonylurea, sulfonylureas, sulfur colloid, sulfur hexafluoride, sulindac, sulphadoxine, sulphadoxine/pyrimethamine, sulphamethoxazole, sulphaphenazole, sulphonylurea, sulpiride, sulthiame, sultiame, Sumaklid, sumatriptan, sumatriptan succinate, sunitinib, Supartz, superoxide dismutase, superparamagnetic iron oxide, SUPREP oral sulphate with gastrografin, suramin, Sureheal, Surgicel, Sustanon, Sustanon 250, suvorexant, suxamethon, suxamethonium, sylimarine, Symfona forte, Systane, Systane Ultra, T-1225, T4P-1001, Tabex, tacrolimus, tadalafil, Taeumjowi-tang, Tagitol V, Talcid, talmapimod, Talwin Nx, Tame, Tamoxifen, tamsulosin, tamsulosin hydrochloride, Tang Ning Tongluo, Tangshen, tapentadol, tariquidar, Tasectan plus, tasimelteon, taurolidine, taurolidine citrate, Taurolock, TauroLock Hep500, tauroursodeoxycholic acid UR-906, taxane, tazarotene, tazobactam, TC-325, Tc-99m DTPA, Tc99m-ethyl cysteinate dimer, TCu380A, Tears Naturale, technetium 99m, Technetium 99m-sulfur, Technetium Tc 99m Sulfur Colloid, technetium Tc 99m tilmanocept, technetium-99, technetium-99m, technetium-99m-diethylenetriamine pentaacetic acid, Technetium-TC99m-labeled Annexin A5, tegafur, tegaserod, Tegeline, telaprevir, telbivudine, telithromycin, telmisartan, temazepam, temozolomide, Tempocol, Tempocol-ColoPulse, temsirolimus, Ten-cha, Tendoactive, tenecteplase, tenofovir, tenofovir disoproxilfumarate, Terbinafine, terbinafine HBF, terbutaline sulphate, teriflunomide, teriparatide acetate, terlipressin, Terminalia arjuna, Testofen, Testosterone, testosterone cypionate, testosterone enanthate, Tetra (4-carboxyphenyl) Porphine (TCPP), tetracaine, tetracaine hydrochloride, tetracosactide hexacetaat, tetracosactrin, tetracycline, tetracycline hydrochloride, Tetradecylthioacetic acid, tetraethylammonium, tetrahydrobiopterin, tetrahydrocannabinol, tetralgin, tetralgin H, Tetrix, Tetronine, Thacapzol, thalidomide, Thealoz, Thealoz Duo, theophylline, theracurmin, Theragran Hematinic, Theramine, TheraSphere, TheraTears, Theraworx, thiamin, thienopyridine, thiocolchicoside, thiocolchicoside SR, thioctic acid, thiopental, thiopentone, thiotepa, Thrombi-Gel, Thrombin-VSI, Thromboreductin, thromboserin, thryoxine, thymidine, Thymoglobulin, Thymol, thymopentin, thymostimulin, Thyroid-stimulating hormone, thyrotropin releasing hormone, thyroxin, Ti02 microfine, Ti02 pigmentary, Tian-qi Jiang-tang, Tianshu capsule, Tian-Wang-Bu-Xin Dan, tiapride, tibolone, ticarcillin-clavulanate, Tigan, tigecycline, timentin, timnodonic acid, timolol, timolol hemihydrate, timolol maleate, timolol maleate in sorbate, Timophtal sine, tinidazole, tinoridine HCl, Tinospora crispa-extract, tinzaparin sodium, Tioth, tiotropium, tiotropium bromide, tipepidine hibenzate, tirapazamine, tirofiban, Tiscover, Tisseel, Tisseel 4 IU/ml VH SID, TISSEEL VH, Tisseela, Tissucol, Tisuacryl, titanium dioxide/zinc oxide, tixocortol, tizanidine, TMD-322, tobramycin, Tobrex, Tocopherol, tocopherol nicotinate, tocopheryl phosphate, tocotrienols, tofacitinib, tokishigyakukagosyuyushokyoto, tolcapone, tolimidone, tolperisone, tolperisone hydrochloride, Tongjiang, Tongxinluo, Tonoferon, Tonsitin, topiramate, topotecan, topotecan hydrochloride, topotecan/carboplatin, torasemide, toremifene, Totilac, trabectedin, tramadol, tramadol hydrochloride, trandolapril, tranexamic acid, tranilast, transamin, transepithelial CXL, transforming growth factor-beta, trans-resveratrol, tranylcypromine, Traumaplant, Traumeel S, Travelan, travoprost, trazodone hydrochloride, tretinoin, triamcinolone, triamcinolone acetate, triamcinolone acetonid, triamcinolone acetonide, triamcinolone hexacetonide, triamsinolone, triazolam, tribulus terrestris, trichloro acetic acid, trichloroacetate, triclosan, Triclosan/Fluoride, triflusal, Trigonella Foenun-Graecum, Trigonelline, triheptanoin, triiodothyronine, Tri-iodothyronine, Tri-luma, Trimedat, trimetazidine, trimethoprim, trimetrexate, trimodal (18)F-choline, Trimo-San, Trinessa, trinitroglycerin, Triomar, Triomune, Triomune 40, triphala, triphala kashaya, triphaladi, Triphalaguduchyadi vati, triptan, triptans, tripterygium, tripterygium glycosides, tripterygium wilfordii, triptophan, trisodium citrate, trisodium citrate dihydrate, trivrut leya, trolamine, TrophAmine, tropisetron, troponin T, troxerutin, trypan blue, tryptophan, TU-025 Keishi Bukuryo Gan, Tualang honey, turmeric, Tylenol, tyramine, Tyrosur, tyrothricin, Tyvera, Ubiquinol, UFT, ulipristal, Ultrapure dialysate, Ultrase MT20, umeclidinium bromide, unacid, unfractionated heparin, uprifosbuvir, Uracyst, Ural-BPH, urea, uridine, urinastatin, Urofleks, Uromax, ursodeoxycholic acid, Urtica dioica, USP endotoxin, Utrogestan, Uvadex, V0034CR01B, Vacyless, Vagifem, Vagipills, Vagisan, VAH-631, valaciclovir, valdecoxib, valerian, valerian mono-extract, Valeriana officinalis L, Valeriane officinalis, Valette, Valexxol, valganciclovir, valproic acid, valpromide, valsartan, valsartan/hydrochlorothiazide, Vamin-J amino acid, vancomycin, vandetanib, vanokserina, vapocoolant, vardenafil hydrochloride, varenicline, vasopressin, vatreptacog alpha, VDO, vecuronium, vecuronium bromide, VEGF-165, VEGF-A165/bFGF plasmid, veliparib, velpatasvir, vemurafenib, Venbig, venlafaxine hydrochloride, Venocur Triplex, Venoferrum, Venozin, Venthamarai chooranam, verapamil, verapamil hydrochloride, Verein, verosudil, vidofludimus, Vigam, Vigantol, Vilac Plus, vilanterol, vilanterol trifenatate, vildagliptin, Vilepi, Vimang, vinblastine, vinblastine sulphate, vincristine, vincristine sulphate, vinegar, vineuro, vinorelbine, Viokase 16, Viscum album homoeopathic mother tincture, viscum fraxinifor, Vismed, vitagnuse, Vitalipin, Vitaliver, vitamin A, Vitamin B12, Vitamin B6, vitamin C, Vitamin D, Vitamin D3, vitamin D3 and calcium, Vitamin E, vitamin K, vitamin K antagonists, vitamin E acetate, Vitano, Vition, Vitreoscilla filiformis, Vivomixx, vleomycin sulphate, VM-26, voglibase, voglibose, Volulyte, Voluven, vonoprazan fumarate, voriconazole, vorinostat, vortioxetine hydrobromide, VP-VI, VSL-3 probiotic, Vyaghrihareetaki avaleha, warfarin, wellbutrin SR, WF-10, WinUBoost, Wobenzym mono, Wobenzym Plus, WS-1442, wuling capsule, Xanmax2002, xanthan gum, xenon, xenon-133, xiaoer qingfei hej, Xin Ran, xiyanping, Xuefu Zhuyu, xylitol, xylometazoline hydrochloride, xylomethazoline, Y-90 hydroxyapatite, Yestimun, Ying Qiao san, Yi Qi Pin Chuan, Yittrium-90, Yohimbine, yohimbine hydrochloride, Yokukansan, Yokukansankachimpihange, yomogi, Yttrium-90, yttrium-90 SIR-spheres microspheres, Z250, Zalain, zalcitabine, zaleplon, Zataria multiflora, zavedos, Ze-91019, Zemuron, Zestra, Zhi Byed 11, zhi ke san, zidovudine, zinc, zinc acetate, zinc gluconate, zinc oxide, zinc pyrithione, zinc sulphate, zincol, Zinga, Zingiber, Zingiber officinale, Zinova, Zintoma, ziprasidone, zofenopril, zoledronic acid, zolmitriptan, zolpidem, Zonnic, and Zyactinase.

### Biologics

In certain embodiments, a stimulus that perturbs a GSC is a biologic selected from (−)-Epigallocatechin 3-gallate, (131)I-rituximab, [1-13C] leucine, [2H5] phenylalanine, 1,4,7,10-tetraazacyclododecane-N, 111In M5A anti-CEA antibody, 111In-capromab pendetide, 12-ATC, 131I-8106, 131I-BC8, 13-cis-retinoic acid, 17 alpha hydroxyprogesterone caproate, 177Lu-DOTATATE, 177Lu-girentuximab, 18 F-fluorothymidine, 18 fluoro-2-deoxyglucose, 18C3, 18F-alovudine, 18F-FDG, 18F-fluorodeoxythymidine, 18F-fluorothymidine, 209-217(210M) peptide vaccine, 213Bi-lintuzumab, 2830929A, 2830930A, 2-methoxyestradiol, 3F8 antibody, 4B5 scFv, 5-flucytosine, 6 mercaptopurine, 6-melanoma helper peptide vaccine, 6-MP, 6-thioguanine, 89Zr-GC-1008, 89Zr-trastuzumab, 90Y-edotreotide, 9-aminocamptothecin, A/H5N1 Antigen, abacavir, abagovomab, abaloparatide, abaloparatide, abarelix, abatacept, abciximab, abetimus, abicipar pegol, abiraterone, abituzumab, abobotulinumtoxinA, ABT-510, acalabrutinib, acarbose, Angiotensin-converting enzyme, acenocoumarol, acetaminophen, acetazolamide, acetylcholine, acetylcysteine, acetylsalicylic acid, aciclovir, acitretin, aclacinomycin, aclarubicin, aclerastide, actoxumab, ACV-1, ACVBP, acyline, Ad26-ZEBOV, Ad35-CS.01, Adacel, adalimumab, adalimumab biosimilar, adecatumumab, adefovir dipivoxil, adenosine, adenovir, adenovirus B7-1, aderbasib, ADH-1, Adhexil, ADM, adrenaline, adrenocorticotropin, adrenomedullin, adriamycin, adriblastin, ADV-TK, AEG-35156, afamelanotide, afatinib, afelimomab, aflibercept, agalsidase alpha, agalsidase beta, agatolimod, Agriflu, AGS-1C4D4, alacizumab pegol, albendazole, albiglutide, albinterferon alpha-2b, albumin, albuterol, Albuterol sulphate, albutrepenonacog alpha, Albutropin, aldesleukin, alefacept, alemtuzumab, alendronic acid, alexamorelin, alphacalcidol, alfimeprase, algenpantucel-L, alglucosidase alpha, alhydrogel, alipogene tiparyovec, alirocumab, alisertib, aliskiren, alisporivir, alitretinoin, Alkeran, Allergovit 6-grasses, Alloferon, allopurinol, all-trans-retinoic acid, alogliptin, alpelisib, alpha 1 proteinase inhibitor, Alpha1-PI, alpha-conotoxins, alpha-galactosidase, alpha-glucosidase, alpha-glucosidase inhibitor, alpha-interferon, alpha-methyldopa, Alphanate, Altastaph, alteplase, alum adjuvant, aluminium hydroxide, aluminum phosphate, ALVAC-1452, ALVAC-CENB7.1, ALVAC-gp100 melanoma vaccine, ALVAC-MART-1 melanoma vaccine, alvocidib, amantadine, amatuximab, amdoxovir, Ametop, AMG-0101, AMG-108, amifostine, amiloride, aminoguanidine, aminopterin, amlodipine, amolimogene bepiplasmid, amoxicillin, amphotericin B, ampicillin, amprenavir, amrubicin, amsacrine, anakinra, anakinra biosimilar, Analatro, anastrozole, anatumomab mafenatox, Anavip, Anbainuo, andexanet alpha, Anfibatide, Angiostatin, anifrolumab, anivamersen, anrukinzumab, anthracycline, anti-IFN alpha, Anti Thymocyte Globulin, anti-CD20-IL2 immunocytokine, anti-CD3 bivalent antibody-diphtheria toxin conjugate, anti-CD3 monoclonal antibody, anti-CD45 mAbs, anti-CEAxanti-DTPA bispecific antibody, anti-IFN gamma, anti-IRL2, antimony, antipsychotics, anti-TAT monoclonal antibodies, antithrombin, antithrombin alpha, antithrombin III, antithymocyte globulin, anti-thymocyte globulin, anti-thymocyte-globulin, anti-T-lymphocyte globulin, anti-TNF alpha, anti-TNF-alpha monoclonals, AP-01, apalutamide, apatorsen sodium, apheresis, apheresis platelets, APITOXIN, apixaban, Apligraf, APN-301, apolizumab, apremilast, aprepitant, aprinocarsen, aprotinin, aquaphor, AR-623, arabinofuranosyl cytidine, ARAC, Ara-C, aracytin, aracytine, ARC-100, ARC-520, argatroban, arginine, arginine vasopressin, argipressin, arsenic trioxide, AS-01 adjuvant, AS-02 adjuvant, AS-02V, AS-03, ascrinvacumab, asfotase alpha, ASN-002, asoprisnil, asparaginase, aspart insulin, aspartame insulin, aspirin, asunaprevir, asunercept, asvasiran sodium, atacicept, atazanavir, atezolizumab, ATG, ATL-101, atorvastatin, atosiban, ATRA, Atrial natriuretic peptide, Atu-027, audencel, Aurograb, avacincaptad pegol sodium, Avanz, Avastin, AVE-1642, aviscumine, Avonex, avotermin, Avrina, axalimogene filolisbac, axitinib, azacitidine, AZA-DR, azathioprine, AZD-1775, azficel-T, azithromycin, azulphidine, Bacillus Calmette Guerin, Bacillus clausii, Bacillus coagulans, bacitracin, baclofen, bacteriophage (Phi-X 174), Bakri Balloon, balapiravir, balixafortide, balugrastim, baminercept, bapineuzumab, barusiban, Basilea, basiliximab, bavituximab, BCG, BCM-95, BCNU, BEAM, becaplermin, beclabuvir, beclomethasone diproprionate, behenoyl-ara-C, belatacept, belimumab, belinostat, bemiparin, bendamustine, benralizumab, benzalkonium chloride, benzylpenicilloyl polylysine, Beriglobin, Beriplast-P, Beriplex P/N, beroctocog alpha, bertilimumab, beta-glucan, beta-hydroxy beta methylbutyrate, betaine, betamethasone, betamethasone dipropionate, Betaseron, bevacizumab, bevacizumab biosimilar, bexarotene, Bexsero, bezlotoxumab, BHQ-880, BHT-3009, bicalutamide, BiCNU, Bifidobacterium breve, bifidobacterium infantis, Bifidobacterium lactis UABLA-12, Bifidobacterium longum, biguanide, BHB-023, bimagrumab, bimekizumab, binimetinib, BioChaperone rhlnsulin, Biolipideo/B2, biotin, biphasic human insulin, biphasic human insulin 30, bisphosphonate, BIT-225, bivalirudin, Bivigam, BL-8040, bleomycin, bleomycin sulphate, bleselumab, blinatumomab, blisibimod, blood cardiplegia, blosozumab, BmAb-200, boceprevir, bococizumab, Boostrix, Boostrix Polio, bortezomib, BOTOX, Botulinum toxin, bovine thrombin, BQ-123, BQ-788, bradykinin, brain natriuretic peptide, brazikumab, bremelanotide, brentuximab vedotin, briakinumab, brimapitide, brimonidine, brivanib alaninate, brodalumab, brolucizumab, bromelain, bromfenac sodium, bromocriptine, bryostatin-1, BSC, BTH-1704, BTI-322, bucelipase alpha, bucillamine, budesonide, budesonide/formoterol, bulsulfex, buparlisib, bupivacaine, burixafor, burlulipase, burosumab, buserelin, buserelin acetate, busulfan, Busulfex, busulphan, butylphthalide, butylscopolammonium bromide, Byclot, Bydureon, cabazitaxel, cabergoline, caboplatin, cad-05, caffeine, CAL, calcineurin inhibitor, calcipotriol, calcitonin, calcitonin gene-related peptide, calcitriol, Caltratate D, Campath, canakinumab, cantuzumab ravtansine, capecitabine, Caphosol, caplacizumab, capromorelin, capsaicin, captopril, carbamazepine, carbetocin, carbimazole, carbohydrate, carbon ion, carboplatin, carfilzomib, carlumab, carmustine, carmustine BCNU, carotuximab, carperitide, caspofungin, catridecacog, catumaxomab, Cavatak, CBP-501, CC-5013, CD34 antibody, CD40 ligand, CDP-870, CDX-1307, CDX-1401, CDX-301, CEA(6D)-TRICOM vaccine, CeaVac, Cedartolen, cefadroxil, cefazolin, ceftaroline fosamil, ceftazidime, ceftriaxone, cefuroxime, celecoxib, celgosivir, celmoleukin, CelTx Apligraf, cenderitide, cenegermin, cenersen, CEpOP+/−R, ceramide, Cerebrolysin, cerliponase alpha, certolizumab pegol, Cervarix, cervical cerclage, cetirizine, cetraben, cetrorelix, cetuximab, CGRP, ChimeriVax-JE, chlorambucil, chlorhexidine, chlorhexidine gluconate, chloroquine, chlorphenamine, chlorpheniramine, chlorpheniramine maleate, chlorthalidone, CHO-Campath-1H, cholecalciferol, cholera vaccine, Chong Kun Dang, CHOP (cyclophosphamide, doxorubicin, vincristine, prednisone), choriogonadotropin alpha, chorionic gonadotropin biosimilar, chorionic gonadotropins, CHP-MAGE-A4, ChronVac-C, CI, cibinetide, ciclosporin, ciclosporine, cilengitide, CimaVax EGF, cimetidine, Cinryze, cintredekin besudotox, Ciprofloxacin, ciprofloxacin hydrochloride, cisplatin, Civacir, cixutumumab, cladribine, Clairyg, clarithromycin, claudiximab, clavulanic acid, clazakizumab, clemastine, clindamycin, clobetasol, clobetasol propionate, clofarabine, clofazimine, clomiphene, clomiphene citrate, clopidogrel, cloxacillin, CML vaccine, CMV peptide N495, CMV pp65 peptide, CMV vaccine, Coagadex, cobimetinib, cocaine, codeine, codrituzumab, colecalciferol, colistin, collagenase, collagenase ABC, coltuximab ravtansine, conatumumab, conbercept, condoliase, conestat alpha, conivaptan, conjugated estrogens, contusugene ladenovec, corifollitropin alpha, corticorelin human, corticosteroid, corticosteroids, corticotrophin-releasing hormone, corticotropin, cosyntropin, cotrimoxazole, co-trimoxazole, C-peptide, CpG-10101, creatine, crenezumab, crisantaspase, crizanlizumab, CroFab, CSII, CSL, CSL-111, CSL's DTPw, CT-102, CTLA4-Ig, CTO, CTS-1027, CultureIle, custirsen, Cutanea, CV-301, Cyclogest, cyclophophamide, cyclophoshamide, cyclophosphamide, cyclophosphamide hydrate, cyclophosphomide, cyclopsorine, cyclosphosphamide, cyclosporin, Cyclosporin A, cyclosporine, cycnocobalamin, cycolophosphamide, cycolphosphamide, cydroxydaunorubicin, Cyklokapron, cylcophosphamide, cyproterone, cyslophosphamide, CYT-107, cytarabine, cytarabine ARA-C, CytoGam, Cytori, cytosine arabinoside, Cytotect, cytoxan, D mannose, dabrafenib, dacarbazine, dacetuzumab, daclatasvir, daclizumab, Dacorin, dactinomycin, dactolisib, Dactylis glomerata pollen, D-Aa-1-peptide-T-amide, dalantercept, dalotuzumab, danaparoid sodium, danazol, danegaptide, danoprevir, dapagliflozin propanediol, dapiclermin, daptomycin, daratumumab, darbepoetin alpha, darunavir, dasabuvir, dasatinib, dasiglucagon, daunomycin, daunorubicin, daunorubicin hydrochloride, daunorubicin liposomal, davalintide, davunetide, DC/I540/KLH vaccine, DCVAC/OvCa, DDAVP, decadron, decitabine, Decortin H, dectrekumab, defibrotide, deflazacort, degarelix, dehydroepiandrosterone, delate synacthen, delavirdine, delcasertib, deleobuvir, demcizumab, denenicokin, Dengvaxia, denileukin diftitox, denosumab, depatuxizumab mafodotin, Depigoid, DermaPro Pep-04, DermaVir, desirudin, desloratadine, deslorelin, desmopressin, desmopressin acetate, desmoteplase, desogestrel, DetoxPC, deuterium-labeled cysteine, dexamethasone, dexchlorpheniramine, DexCom G4 platinum, dexmedetomidine, dexrazoxane, dextromethorphan, dextromethorphan hydrobromide, dextrose, Diamyd, dianexin, DiaPep-277, diaspirin crosslinked hemoglobin, diazoxide, dibotermin alpha, dichloroacetate, didanosine, di-DTPA-131I peptide, difelikefalin, Diffistat-G, Digibind, Dimericine, dimesna, dimethyl fumarate, dimethyl sulfoxide, dinoprostone, dinutuximab, dinutuximab beta, diphenhydramine, dipyridamole, diroton, dirucotide, disitertide, DITC, ditiazem, DKN-01, DMARD, DNA/MVA.HBs, DnaK, DNE-3, docetaxel, dociparstat, doconexent ethyl ester, dolcanatide, donepezil hydrochloride, dopamine, dorgenmeltucel-L, dornase alpha, doxorubicin, doxorubicin HCl liposome, doxorubicin hydrochloride, doxorubicin-antibody conjugate, doxorubicin-BR96 conjugate, doxycycline, DPP-4 inhibitor, DRibbles vaccine, drospirenone, drotrecogin alpha, drozitumab, DT, DT PACE, DTaP, DTGM, DTP3, DTP3a-IPV-HBV/HIB vaccine, DTP3a-IPV-HIB, DTP4, DTPa, DTPa vaccine, DTPwcsl, DTPw-HBV Kft., DTwP—HBV-HIB vaccine, Dukoral, dulaglutide, dulanermin, duligotuzumab, dupilumab, durvalumab, dusigitumab, dutasteride, duvelisib, E coli endotoxin, E7 peptide, ebenatide, ecallantide, echinocandin, Ecosprine, ecromeximab, eculizumab, Ecural, edaravone, edifoligide, edobacomab, edodekin alpha, edratide, edrecolomab, EF-1 peptide, EF-2 peptide, efalizumab, efavirenz, eflapegrastim, efmoroctocog alpha, efpeglenatide, eftrenonacog alpha, efungumab, egaptivon pegol, elbasvir, elcatonin, eldecalcitol, eldelumab, elemental calcium, elgemtumab, elisidepsin, Elocom, Elocon, Elomet, Elosalic, elosulfase alpha, elotuzumab, elpamotide, elsiglutide, eltrapuldencel-T, eltrombopag, emapticap pegol, Emdogain, emepepimut-S, emfilermin, emibetuzumab, emicizumab, empegfilgrastim, emtricitabine, EMZ-701, enalapril, endolipide, endothelin-1, enfuvirtide, Engerix B, enocitabine, enokizumab, enoxaparin sodium, entecavir, enteric-coated mycophenolate sodium, entinostat, enzalutamide, enzastaurin, EP-100, epacadostat, ephedrine, Epifasi, epinephrine, epirubicin, epoetin, epoetin alpha, epoetin alpha biosimilar, epoetin alpha BS, epoetin alpha or beta, epoetin beta, epoetin delta, epoetin omega, epoetin theta, epoetin-A, Eposin, epratuzumab, eptacog alpha, eptifibatide, eptinezumab, eptotermin alpha, Equine antithymocyte immunoglobulin, equine ATG, erenumab, ergometrine, ergonovine, ergonovine maleate, eribulin mesylate, erlotinib, ertugliflozin, ertumaxomab, erythrocyte stimulating agents, erythromycin, erythropoietin, erythropoietin biosimilar, erythropoietin follow-on biologic, erythropoietin receptor activator, erythropoietin stimulating agents, ESBA-105, Escherichia coli L-asparaginase, esomeprazole, estetrol, estrace, estradiol, estradiol acetate, estradiol valerate, estramustine, estramustine phosphate sodium, estriol, estriol E3, estrodiol, estrogen, estroprogestins, etanercept, etanercept biosimilar, etaracizumab, etelcalcetide, ethinyl estradiol, ethinylestradiol, ethyl hydrogen fumarate calcium, ethyl hydrogen fumarate magnesium, ethyl hydrogen fumarate zinc, etirinotecan pegol, etoposide, etoposide phosphate, etravirine, etrolizumab, Eudragit-L-coated mesalamine, everolimus, evofosfamide, evolocumab, examorelin, exbivirumab, exemestane, exenatide, exendin, exendin-4, exendin-9, Exsulin, Extavia, ezetimibe, F18, F-18 fluorodeoxyglucose, F-627, Factor VIII, Factor VIII concentrate, Factor VIII/von Willebrand Factor complex, Factor XIII concentrate, faldaprevir, famotidine, Fanhdi, farletuzumab, fasinumab, fat-soluble vitamins, FE[50]C, FE[75]C, FEBA, FEIBA NF, fenofibrate, fenretinide, fentanyl, Feron, ferric gluconate, ferric saccharose, ferrous fumarate, ferrous sulphate, fesoterodine fumarate, fetal fibronectin, fexapotide triflutate, fexofenadine, fezakinumab, fibatuzumab, fibrinogen, fibrinogen concentrate, ficlatuzumab, fidaxomicin, figitumumab, filanesib, filgrastim, filgrastim biosimilar, filgrastim biosimilar API, filgrastim SD/01, filibuvir, fingolimod, Flagyl, Flebogamma DIF, fletikumab, flotegatide, floxuridine, flt3-ligand, Fluarix Quadrivalent, fluciclovine, flucloxacillin, fluconazole, flucytosine, fludarabine, fludarabine monophosphate, fludarabine phosphate, fludeoxyglucose, fludeoxyglucose F 18, fludrocortisone, Flufirvitide, flunarizine, fluocinolone acetonide, fluoraouracil, fluoro-L-thymidine, fluorometholone, fluoropyrimidine, fluorouracil, fluoxetine hydrochloride, flupirtine, flutamide, fluticasone propionate, fluvastatin, Fluvirin, Fluzone, FMISO, FMP1/ASO2A, Folacin, FOLFIRI, FOLFIRINOX, folic acid, folinate, folinic acid, follicle stimulating hormone, Follistim, follitropin alpha, follitropin alpha biosimilar, follitropin beta, follitropin delta, fomivirsen, fondaparinux sodium, fontolizumab, foralumab, foravirumab, forigerimod, formoterol, fosamprenavir, fosbretabulin, foscarnet, fosphenytoin, fostamatinib, Fostipur, fotemustine, Fovepta, fowlpox-CEA-TRICOM vaccine, fowlpox-PSA vaccine, FP9 ME-TRAP, fragmin, fresolimumab, Freund's adjuvant, FSH, FSH-GEX, fulranumab, fulvestrant, furosemide, fusidic acid, G-4460, gadolinium, gadolinium-diethylenetriamine penta-acetic acid, gadopentetate dimeglumine, galactose, galantamine, galcanezumab, galinpepimut-S, galiximab, gallium nitrate, galsulfase, gamma-globulin, Gammaplex, Gamunex, ganciclovir, ganciclovir phosphonate, ganetespib, ganirelix acetate, ganitumab, Gardasil, Gardasil 9, gataparsen, gavilimomab, GCS-100, GCSF, G-CSF, Gd-MRI, gedatolisib, gefitinib, Gelsolin, gemcitabine, gemtuzumab, gemtuzumab ozogamicin, gentamicin, gentamicin sulphate, gevokizumab, ghrelin, GI-4000, GI-5005, gimeracil, girentuximab, Glamin, glatiramer acetate, glembatumumab vedotin, glepaglutide, glibenclamide, gliclazide, gliclazide MR, glimepiride, glipizide, GLP-1, GLP-1 analogues, GlucaGen, glucagon, glucagon like peptide-1, glucagon-like peptide-1, glucagon-like peptide-1-albumin recombinant protein, glucarpidase, glucocerebrosidase, Glucose dependent insulinotropic polypeptide, glucose oxidase, Glucose potassium insulin, GlutaDON, glutamic acid, glutamine, Glutoxim, glycerin trinitrate, glyceryl trierucate, glyceryl trioleate, Glymera, Glypromate, GM-CSF, GM-CSF vaccine, GM-CT-01, GnRH, GnRH agonist, GnRH antagonist, golimumab, golnerminogene pradenovec, gonadotrophins, gonadotropin, gonadotropin releasing hormone, gonadotropin releasing hormone agonist, gonadotropin releasing hormone antagonist, Gonadotropin-releasing hormone, goserelin, goserelin acetate, gp100, gp100 17-25, gp100 209-217(210M), gp100 antigen, gp100 antigen/tyrosinase antigen, gp100 peptides, gp100:209-217(210M) peptide vaccine, gpASIT+, granulocyte colony-stimulating factor, Granulocyte-macrophage colony-stimulating factorr, Grazax, grazoprevir, GS-4774, GS-9256, GSK-1437173A, GSK-208108, GSK-2189242A, GSK-2197870A, GSK-2202083A, GSK-2302024A, guadecitabine sodium, guselkumab, gusperimus trihydrochloride, GVAX, H-101, H-1299, H1N1 pandemic influenza vaccine, H5N1 influenza vaccine, HAART, Haemaccel, Haemate P, hANP, Haporine-S, Havrix, HBV, HBV-MPL 208129, HCG, HDMTX, Hearticellgram, Hecoria, Hemabate, HemaMax, Hemate P, hemicellulose, hemoglobin glutamer 250, hemoglobin raffimer, Hemospan, Hemospray, HepaGam B, heparin, heparin sodium follow-on biologic, heparinoid, hepatitis B immunoglobulin, Hepavax-Gene, HerpV, Hexaxim, HIB vaccine, HIB-MCVc, Hib-MenCY-TT, HinsBet, histamine dihydrochloride, histrelin, HIV-1G, Hizentra, HL-143, hLF 1-11, HMG, HMG-CoA reductase inhibitor, hMG-IBSA, HMG-Lepor, HOE21PH insulin, horse antithymocyte globulin, HPbetaCD-I complex, hpFSH, hpHMG, HP-hMG, HRT-FER, hTERT D988Y peptide, hTERT 1540 peptide, hTERT R572Y peptide, human albumin, human anti-thymocyte globulin, human atrial natriuretic peptide, human BNP-32, human chorionic gonadotrophin follow-on biologic, human chorionic gonadotropin, human fibrinogen, human growth hormone, human immune globulin, human insulin, human insulin mix 30/70, human menopausal gonadotropin, human normal immunoglobulin, human parathyroid hormone, human protein C, human recombinant hyaluronidase, human recombinant insulin, human secretin, human serum albumin, Human serum albumin 20, human thrombin, Humate P, HuMax-IL8, hyaluronate sodium, hyaluronic acid, hyaluronidase, hydralazine, hydrochlorothiazide, hydrocortisone, hydrocortisone sodium succinate, hydroxycarbamide, hydroxychloquine, hydroxychloroquine, hydroxydaunorubicin, hydroxydaunorubicin hydrochloride, hydroxyethyl starch, hydroxyprogesterone caproate, hydroxyrubicin, hydroxyurea, hydroxyzine, Hylenex, hyperfractionated cyclophosphamide, ibalizumab, ibandronic acid, iboctadekin, ibritumomab tiuxetan, ibrutinib, ibuprofen, Ibutilide, icatibant, icodextrin, icosapent ethyl ester, icrucumab, idarubicin, idarucizumab, idelalisib, Id-KLH, idursulfase, IDX-184, IFN-alpha, IFN-beta, IFN-gama-1b, IFN-gamma, ifosafamide, ifosamide, Ifosfamide, Ig NextGen 10%, Ig VENA, Igantibe, IGIV3I, 11V, IL-15, IL-2, IL-4R, ilodecakin, iloprost, I-LV, IMA-901, IMA-910, IMA-950, ImaginAb, imalumab, imatinib, imetelstat, imgatuzumab, imiglitazar, imiglucerase, imiglucerase biosimilar, Iminoral, imipramine, imiquimod, ImmTher, ImMucin, ImmuCyst, Immuncell-LC, immuneGlobulin, immunocyanin, immunoglobulin, IMO-2055, IMO-2125, IMO-8400, IMP-321, Imprime PGG, Imuran, inclacumab, incobotulinumtoxinA, incomplete Freund's adjuvant, indinavir, indium In 111 monoclonal antibody MN-14, indium-111, indocyanine green, indomethacin, inebilizumab, infliximab, infliximab biosimilar, Influvac TC, inogatran, inotuzumab ozogamicin, Insujet, insulatard, insulin, insulin aspart, insulin aspart protamine, insulin aspart protamine 30/70, insulin degludec, insulin detemir, insulin glargine, insulin glargine biosimilar, insulin glulisine, insulin insulatard, insulin isophane, insulin lispro, insulin lispro biosimilar, insulin NPH, insulin peglispro, insulin tregopil, Intanza, Intercell, interferon, interferon alpha, interferon alpha-2a, interferon alpha-2b, interferon alphacon-1, interferon alpha-n1, interferon alpha-n3, interferon beta, interferon beta-1a, interferon beta-1a follow-on biologic, interferon beta-1b, interferon beta-1b follow-on biologic, interferon gamma, interferon gamma-1b, Interferon-beta, interleukin 2, interleukin-1, interleukin-1 receptor antagonist, Interleukin-10, interleukin-11, interleukin-12, interleukin-2, interleukin-4, Intragam P, iodine I 125, Iodine I 131, iodine-123 metaiodobenzylguanidine, iodine-131, iodixanol, IONIS-GCGRRx, IONIS-PTP1BRx, ioxaglate, ipamorelin, iphosphamide, ipilimumab, I-Port, ipragliflozin, IPV-AI SSI, iratumumab, irinotecan, irinotecan sucrosofate, IRIS, iron, iron dextran, IRX-2, IRX-5183, ISA-102, ISIS-14803, isoflurane, isofomide, isoniazid, isophosphamide, isosorbide dinitrate, isosorbidedinitrate, isotretinoin, isunakinra, itolizumab, itraconazole, ixabepilone, ixekizumab, JNJ-56914845, Jumtab, Kedbumin, keratinocyte growth factor, ketoconazole, ketorolac, Keyhole-Limpet Hemocyanin, Kidrolase, Kinevac, Kinrix, KLH, kollagenase, KSL-W, L19-IL-2, L19-TNF-alpha, labetuzumab, Lactated Ringers, Lactobacillus acidiphilus, Lactobacillus acidophilus DDS-1, Lactobacillus fermentin, Lactobacillus fermentum CECT5716, Lactobacillus fermentum LC40, Lactobacillus reuteri CR20, Lactobacillus reuteri RC-14, Lactobacillus rhamnosus GR-1, Lactobacillus salivarius, Lactobacillus salivarius HN6, lactoserum, Lais, lamivudine, lampalizumab, lancovutide, landogrozumab, lanoteplase, lanreotide, lanreotide acetate, lansoprazole, lapatinib, laquinimod, larazotide acetate, L-arginine, laromustine, laronidase, larotaxel, L-asparaginase, latiglutenase, LBVW-0101, LC-280126, lebrikizumab, ledipasvir, lefitolimod, leflunomide, leishmaniasis vaccine, lenalidomide, lenograstim, lenzilumab, lepirudin, Leptin, leridistim, lestaurtinib, letrozole, Leucotropin, leucovorin, leucovorin calcium, leuprolide, Leuprone, leuprorelin acetate, Leuvectin, levamisole, levetiracetam, levobupivacaine, levocarnitine, levocetirizine, levofloxacin, levo-folinate, levofolinic acid, levonorgestrel, levothyroxine sodium, lexaptepid pegol, LH, liatermin, libivirumab, lidocaine, ligelizumab, lignocaine, linaclotide, linagliptin, linezolid, linsitinib, lintuzumab, liothyronine sodium, lipegfilgrastim, Lipopoolysaccharide, liposomal amphotericin B, liposomal cytarabine, liposomal doxorubicin, liposomal interleukin-2, liposome-based adjuvant ASO1B, liprotamase, liraglutide, lisinopril, lithium, litronesib, lixisenatide, L-Leucine, L-leucovorin, LMB-2, L-menthol, LN-145, L-NMMA, lomibuvir, lomustine, lonafarnib, long-acting beta-2-agonist, long-acting insulin, long-acting polysialic acid-erythropoietin conjugate, Long-chain triglycerides, lonoctocog alpha, lopinavir, LOR-2040, loratadine, lorazepam, lorvotuzumab mertansine, losartan, loteprednol etabonate, lovastatin, low molecular weight sulphated dextran, LPS endotoxin, Lubriderm, lucatumumab, lucinactant, lumiliximab, luminespib, lumretuzumab, LungVax, Lupron, luspatercept, lusupultide, lutein, luteinizing hormone, luteinizing hormone releasing hormone agonist, lutropin alpha, LY-2181308, lycopene, lysine, mAb anti-CD3 conjugated to recombinant ricin A chain, mAb anti-CD7 conjugated to recombinant ricin A chain, Macrophage colony-stimulating factor, MAGE-1.A2, MAGE-10.A2, MAGE-3, MAGE-3.A2, MAGE-4.A2, MAGE-C2.A2, mapatumumab, maraviroc, MART-1 antigen, MART-1 peptide vaccine, masitinib, matuzumab, mavrilimumab, MBL-HCV1, MCVc vaccine, MDRNA, MDX-1379, MDX-CTLA4, mecasermin, mecasermin rinfabate, MEDT-551, MEDT-7510, Medihoney, medroxyprogesterone, medroxyprogesterone 17-acetate, medroxyprogesterone acetate, megestrol, Melan-A, melatonin, Melaxin, meloxicam, melphalan, melphalan flufenamide hydrochloride, Menactra, MenAfriVac, Mencevax, Meningitec, Menitorix, Menogon, Menopur, menotropin, menotropins, Menveo, mepivacaine, mepolizumab, mercaptopurine, mericitabine, merimepodib, Merional, meropenem, mesalamine, mesalazine, mesna, metelimumab, metenkefalin, metenkefalin acetate, metformin, metformin hydrochloride, methadone, methimazole, methotexrate, methotrexate, methoxsalen, methoxy polyethylene glycol-epoetin beta, methyl prednisolone, methylphenidate hydrochloride, methylprednisolone, methylprednisone, methyprednisolone, metranidazole, metreleptin, metronidazole, MF-59, micafungin, MicOryx, microbial nanocellulose, micronized progesterone, Microrelin, midazolam hydrochloride, midismase, midodrine, mifepristone, migalastat, miglitol, miglustat, milatuzumab, milatuzumab-doxorubicin conjugate, minocycline, Minprostin, mipsagargin, mirostipen, misoprostol, mitiglinide, Mitizax, mitomycin, mitomycin C, mitotane, mitoxantrone, mitoxantrone hydrochloride, mitumprotimut-t, mizoribine, MK-0941, MK-2206, MK-3641, MK-8226, MM-111, MM-121, MM-121+paclitaxel+doxorubicin+cyclophosphamide, MMR vaccine, M-M-RII, MMRV vaccine, M-M-RVAXPRO, MoAbs, MOAD, mocetinostat dihydrobromide, modimelanotide, mogamulizumab, molgramostim, mometasone, mometasone furoate, monalizumab, monoclonal antibody CD19, monoclonal antibody CD20, monoclonal antibody Mel-14 F(ab′)2, monotard, Montanide, montanide ISA 51 VG, Montanide ISA51, montanide ISA-51, montelukast sodium, monteplase, moroctocog alpha, moroctocog alpha biosimilar, morphine, mosapride, motavizumab, motesanib diphosphate, motexafin gadolinium, moxetumomab pasudotox, MP, MPR, mRNA-gp100, mRNA-Mage-A1, mRNA-Mage-A3, mRNA-Melan-A, mRNA-survivin, mRNA-tyrosinase, MT-2301, MUC-1-KLH vaccine, multiclade DNA recombinant adenovirus-5, multi-epitope tyrosinase/gp100 vaccine, Multiferon, Multifocal ERG, Multikine, muromonab-CD3, mutant butyrylcholinesterase-albumin fusion protein, MVA ME-TRAP, MVA-BN Filo, M-Vax, mycophenolate acid, mycophenolate acid sodium, mycophenolate mofetil, mycophenolate sodium, mycophenolic acid, Myeloma Immunoglobulin Idiotype Vaccine-KLH, Myrcludex B, N′,N″,N′″-tetraacetic acid, NA17.A2, Nabi-HB, N-acetylcysteine, nafarelin, NAHE-001, naloxone, namilumab, Nanogam, naproxen, naptumomab estafenatox, narlaprevir, natalizumab, nateglinide, natrium chloride, navitoclax, NDDPX-08, Necator americanus, necitumumab, nedaplatin, neihulizumab, NeisVac-C, nelarabine, nelfinavir, nelipepimut-s, nemifitide, nemolizumab, Neoral, Neoveil, nepidermin, nesbuvir, nesiritide, Nesp, Neublastin, Neucardin, Neupogen, Neurim, Neurotropin, Neurovision, neutrazumab, nevirapine, NewGam, Nexfin, NGM-282, NGR-hTNF, NHS-IL2-LT, nicorandil, nicotine, NicVAX, nifedipine, Niferex, nilotinib, nilutamide, Nimenrix, nimotuzumab, nimustine hydrochloride, nintedanib, nirogacestat, nitazoxanide, nitrazepam, nitric oxide, nitroglycerin, Nitromex, nitroprusside, nitrous oxide, Niuliva, nivobotulinumtoxin A, nivolumab, NN-1953, N-Nitro-L-Arginine Methyl Ester, Nobex, nonacog alpha, nonacog alpha biosimilar, nonacog beta pegol, nonacog gamma, non-pegylated granulocyte-colony stimulating factor, non-pegylated liposome-encapsulated doxorubicin, noradrenaline, Norelin, norepinephrine, norethindrone, norethindrone acetate, norethisterone, norethisterone acetate, norfloxacin, NovaCaps, Novaferon, novosis, NovoTTF-100A, NPH insulin, NPH/Regular insulin, NPO-11, nucleoside/nucleotide analog, Nuleusin, Nutropin, NY-ESO-1 vaccine, NY-ESO-1.A2, nystatin, obatoclax, obinutuzumab, oblimersen, ocaratuzumab, ocrelizumab, ocriplasmin, Octafibrin, Octagam, Octaplex, octocog alpha, octreotide, octreotide acetate, octreotide hydrochloride, octreotide LAR, OCV-101, ofatumumab, ofloxacin, OHR-118, OKT3, olaptesed pegol, olaratumab, oligodeoxynucleotides, olokizumab, olopatadine, olopatadine hydrochloride, omacetaxine mepesuccinate, omalizumab, omarigliptin, ombitasvir, omega interferon, Omegaven, omeprazole, omiganan, omiganan pentahydrochloride, Omnitrope, Omr-IgG-am, onabotulinumtoxinA, onartuzumab, once-daily naproxen, Onco-TCS, OncoVAX, Oncovin, Oncovir, ondansetron, onercept, ontuxizumab, opebacan, opicinumab, oportuzumab monatox, oprelvekin, oprozomib, Optivate, Optive, OPV3, OPV4, Oralgam, oregovomab, orlistat, Orthovisc, oseltamivir, OSH-101, osimertinib, Ostenil, otelixizumab, oteracil potassium, otlertuzumab, OTR-4120, oxaliplatin, oxaloacetic acid, oxandrolone, Oxazyme, oxelumab, oxybutynin, oxygen, oxyntomodulin, oxytocin, ozanezumab, ozarelix, ozoralizumab, P1A, P-3072, P-3073, paclitaxel, paclitaxel trevatide, pacritinib, pagibaximab, palifermin, palivizumab, palladium Pd 103, pamidronate disodium, pamrevlumab, Pancrease MT, pancreatin, pancrelipase, panitumumab, panobacumab, panobinostat, pantoprazole, paracetamol, paricalcitol, paritaprevir, paromomycin, parsatuzumab, pascolizumab, pasireotide diaspartate, pasireotide pamoate, pateclizumab, patritumab, pazopanib, PCV7, PDGF-BB, Pediarix, Pediasure, pednisone, PedvaxHIB, pegapamodutide, pegaptanib, pegargiminase, PEG-asparaginase, pegaspargase, Pegasys, PEG-bHb-CO, pegcrisantaspase, pegdinetanib, PEG-FGF21, pegfilgrastim, pegfilgrastim biosimilar, PEG-glucocerebrosidase, peg-granulocyte-colony stimulating factor, PEG-IFN, peg-IFN-alpha, Peg-IFN-alpha2a, PEG-IFN-SA, peg-ilodecakin, peginesatide, peg-INF, peginterferon alpha, peginterferon alpha-2a, peginterferon alpha-2a biosimilar API, peginterferon alpha-2a follow-on biologic, peginterferon alpha-2b, peginterferon alpha-2b follow-on biologic, peg-interferon alpha, peginterferon alpha 2, peg-interferon alpha-2a, peg-interferon alpha-2b, peginterferon beta-la, peginterferon lambda-la, PEG-interleukin-2, pegloticase, PEG-loxenatide, pegmusirudin, pegnivacogin, pegpleranib sodium, PEG-rASNase, pegsunercept, pegteograstim, pegvaliase, pegvisomant, PEGylated hyaluronidase, Pegylated IFN-alpha 2B, pegylated IFN-alpha-2a, pegylated intereferon alpha, pegylated interferon, pegylated interferon alpha-2a, pegylated interferon alpha, pegylated interferons, pegylated liposomal doxorubicin, PEGylated recombinant human growth hormone, PEGylated recombinant staphylokinase variant, pegylated-GCSF, pegylated-G-CSF, pegylated-IFN-alpha, pegylated-interferon-alpha, pelareorep, pembrolizumab, pemetrexed, pemetrexed disodium, penicillin, Pentacel, pentamidine, pentavalent rotavirus vaccine, Pentaxim, pentetic acid calcium, pentostatin, pentoxifylline, perfluoropropane, Perioperative Corticosteroids, personalized recombinant protein vaccines, pertuzumab, pevonedistat, pexastimogene devacirepvec, pexelizumab, pexiganan acetate, phenergen, pheniramine maleate, phentermine, phenylbutyrate, phenylephrine, phenytoin, PhiX 174, picibanil, pictilisib, pidilizumab, pilaralisib, pioglitazone, piperacillin, pirarubicin, pitavastatin, pitrakinra, Pituitary adenylate cyclase-activating polypeptide-38, pixantrone, Pixykine, placebo, placulumab, PlaMavax, plasma, Plasmin, Plasmodium vivax vaccine, platelets, platinum, plecanatide, plerixafor, plitidepsin, plovamer acetate, plozalizumab, Pneumaococcal Vaccine, Pneumo Novum, pneumococcal polyvalent vaccine, Pneumovax 23, polidocanol, polifeprosan 20 with carmustine, Poliorix, poliovirus vaccine, polyclonal IgY antibody, polyglutamate camptothecin, polyglycolic acid sheets, polymyxin B, polysaccharide-K, polysialic acicVKLH/QS-21 vaccine, pomalidomide, ponezumab, POT-4, potassium, potassium chloride, povidone iodine, povidone-iodine, poziotinib, PPSV23, pramlintide, pramlintide acetate, prasugrel, pravastatin, Pred Forte, Pred Mud, prednisolon, prednisolone, prednisone, prednsione, pregabalin, Pregnyl, Premarin, Premixed nitrous oxide, Prempro, Prevnar, Prevnar 13, prezatide copper acetate, PRHgh, prilocaine, Priorix, Priorix-Tetra, Privigen, probiotic, probiotics, procarbazine, procarbazine hydrochloride, progesterone, Progesterone in oil, progestin, Prolastin, Prolastin-C, Proleukin, promethazine, Prontogest, proparacaine, propranolol, propylthiouracil, ProQuad, Prosaptide TX14(A), Prosigne, prostaglandin F2alpha, protamine, protaphane, prourokinase, Provera, PTH(1-34), PUFA, PVX-410, PXFK peptide, pyrazinamide, pyridoxine, QS-21, Quadracel, quilizumab, quinine, Quintanrix, quizartinib dihydrochloride, rAAT, Rabies immunoglobulins, racotumomab, radalbuvir, radioactive iodine, radiolabeled anti-PSMA huJ591 minibodies, rafivirumab, ragaglitazar, raloxifene, raltegravir, raltitrexed, ramatercept, ramipril, ramoplanin, ramucirumab, ranibizumab, ranibizumab biosimilar, ranimustine, ranitidine, ranpirnase, rapamycin, rapastinel, Rapid acting regular insulin(correction factor), rasburicase, RATG, RAV-12, raxibacumab, Rebidose, Rebif 22, Rebif 44, Rebif Rebidose, recAAT, recFSH, Recombinant adenovirus-herpes simplex virus thymidine kinase, recombinant adenovirus-poxvirus prime-boost malaria vaccine, recombinant antithrombin III, recombinant factor VIII, recombinant flt3 ligand, recombinant follicle stimulating hormone, recombinant fowlpox GM-CSF vaccine adjuvant, recombinant fowlpox-CEA(6D)/TRICOM vaccine, recombinant fowlpox-prostate specific antigen vaccine, recombinant FSH, recombinant GM-CSF, Recombinant growth hormone, Recombinant HBsAg with different concentrations of aluminium salts and/or MPL, recombinant HBV vaccine, recombinant hCG, recombinant hepatitis B vaccine, recombinant human alpha B-crystallin, recombinant human alpha-fetoprotein, recombinant human arylsulfatase A, recombinant human chorionic gonadotropin, recombinant human endostatin, recombinant human epoetin alpha, recombinant human erythropoietin, recombinant human fibroblast growth factor type 2, Recombinant human follicle stimulating hormone, recombinant human granulocyte colony stimulating factor, recombinant human growth hormone, recombinant human hyaluronidase, recombinant human iduronate-2-sulfatase, Recombinant human IGF-1, recombinant Human IL-15, recombinant human insulin, Recombinant human intrinsic factor, recombinant human keratinocyte growth factor-2, recombinant human lactoferrin, recombinant human parathyroid hormone, recombinant human thrombopoietin, recombinant hypoallergenic vaccine, recombinant insulin glargine, recombinant interferon alpha, Recombinant interferon alpha-2a, recombinant interferon alpha-2b, recombinant interferon beta, recombinant interferon beta-la, recombinant interferon gamma, recombinant interleukin-12, recombinant interleukin-2, recombinant interleukin-3, recombinant LH, recombinant luteinizing hormone, recombinant lysostaphin, recombinant midismase, recombinant moroctocog alpha, recombinant prolactin, recombinant streptokinase, recombinant thymosin alpha 1, recombinant thyroid-stimulating hormone, recombinant tissue plasminogen activator, recombinant tumor necrosis factor family protein, recombinant vaccinia prostate-specific antigen vaccine, recombinant vaccinia-CEA(6D)-TRICOM vaccine, Recombinate, Recombivax HB, reconstituted HDL, Reditux, refanezumab, Refresh Endura, regorafenib, relamorelin, Rellidep, reltecimod, relugolix, remtolumab, repaglinide, reslizumab, RespiGam, resveratrol, retaspimycin, reteplase, retinoic acid, retinyl palmitate, reveglucosidase alpha, rexlemestrocel-L, rFSH, r-FSH, RG-6149, Rh-Apo2L, rhBMP-2, rhBMP-2/ACS/INTER AX, rhGDF-5, rhIL-11, rhIL-12, rhPDGF-BB, rhTPO, rhuCD40L, rHuEPO, r-Hu-EPO, R-hyperCVAD, ribavirin, ribaxamase, riboflavin, ridaforolimus, ridogrel, rifabutin, rifampicin, rifampin, rIFN-g, rilonacept, rilotumumab, rilpivirine hydrochloride, riluzole, rimabotulinumtoxinB, rindopepimut, Ringer lactate, rintatolimod, risankizumab, risedronate sodium, ritodrine, ritonavir, rituximab, rituximab biosimilar, rivaroxaban, r-LH, rNAPc2, RO-4929097, robatumumab, rocapuldencel-T, Roferon-A, roflumilast, romiplostim, romiplostim biosimilar, romosozumab, rontalizumab, ropeginterferon alpha-2b, ropivacaine, rosiglitazone, rosiglitazone maleate, rosomidnar, rosuvastatin, Rotarix, RotaTeq, rotigaptide, rovelizumab, rozrolimupab, RPI-78M, r-proUK, rT-PA, ruplizumab, rurioctocog alpha pegol, rusalatide, ruxolitinib, rV-PSA, RX-0201, Sabin-IPV, S-adenosyl-L-methionine, sagopilone, salbutamol, saline, salmon calcitonin, samalizumab, samarium(153Sm) lexidronam, SAMe, saquinavir, sargramostim, sarilumab, satraplatin, saxagliptin, SB-743921, Sch-58500, SCY-078, SCY-635, sebelipase alpha, secnidazole, second-generation leptin analogs, secukinumab, selenium, selonsertib, selumetinib, semaglutide, semaxanib, Se-methyl-seleno-L-cysteine, semuloparin, senrebotase, sepantronium bromide, serelaxin, seribantumab, setmelanotide, setrobuvir, sevirumab, SGN-30, sibrotuzumab, sibutramine, sifalimumab, siG12D LODER, sildenafil citrate, silibinin dihydrogensuccinate, siltuximab, silver sulfadiazine, simeprevir, simoctocog alpha, simtuzumab, simvastatin, sincalide, siplizumab, sipuleucel-T, sirolimus, SIR-Spheres, SIR-Spheres microspheres, sirukumab, sitagliptin, sitimagene ceradenovec, soblidotin, sodium aurothiomalate, sodium hyaluronate, sodium nitroprusside, sodium stibogluconate, sodium thiosulphate, sofosbuvir, solanezumab, solnatide, SomaDex, somapacitan, somatostatin, Somatotropin, somatrogon, somatropin, somatropin biosimilar, somatropine, somavaratan, sonepcizumab, sonolisib, sorafenib, sotatercept, sotrastaurin, sovaprevir, SPI-1620, spironolactone, sprifermin, squalamine, stamulumab, stavudine, Stereonate, sterile sodium hyaluronate solution, steroids, streptokinase, streptozocin, streptozotocin, strontium chloride Sr 89, Subcuvia, succinobucol, sulfamethoxazole, sulfasalazin, sulfasalazine, sulfonated human immunoglobulin, sulfonylurea, sulindac, sulphamethoxazole, sulphonylurea, sunitinib, suprachoroidal CLS-TA, suramin sodium, surotomycin, survivin peptide, survivin Sur1M2 peptide, synacthen, Synflorix, Synthroid, Syntocinon, Syntometrine, Syntommetrine, tabalumab, TA-CIN, tacrolimus, tadalafil, talabostat, talactoferrin alpha, taliglucerase alpha, talimogene laherparepvec, tamoxifen, tamoxifen citrate, tamsulosin hydrochloride, tandutinib, tanezumab, tarextumab, taribavirin hydrochloride, tasonermin, taspoglutide, tasquinimod, Tauferon, taurine, taxanes, taxol, tazobactam, TC-3, TC-3 hydrogel, TCM-700C, Tdap vaccine, Td-IPV, Tdvaccine, teduglutide, tefibazumab, tegafur, tegafur-gimeracil-oteracil potassium, tegafur-uracil, tegobuvir, telaprevir, telavancin, telbermin, telbivudine, temozolomide, temsirolimus, tenecteplase, teniposide, tenofovir, tenofovir disoproxil fumarate, teplizumab, teprotumumab, Terap C, terbutaline, tergenpumatucel-L, teriflunomide, teriparatide, teriparatide biosimilar, terlipressin, tertomotide, tesamorelin, tesidolumab, testosterone, testosterone cypionate, testosterone enanthanate, testosterone enanthate, Tetanol, tetanus diphtheria toxoids, tetanus toxoid, tetracaine, tetracosactide, tetracosactide hexaacetate, Tetravisc, teverelix, tezepelumab, TG-4010, TG-4040, tgDCC-E1A, TGF-beta 2, Thal/IFN, thalidomide, theophylline, Theratope, thiazolidinedione, thioguanine, thiotepa, thrombin, thrombin alpha, Thrombogenics, thrombolysis, thrombomodulin alpha, thrombopoietin, Thrombuster H, thymalphasin, thymoglobulin, thymopentin, thyrotropin alpha, thyrotropin-releasing hormone, thyroxine, thyroxine and triiodothyronine, tiagabine, ticagrelor, tifacogin, tigapotide, tigatuzumab, tildrakizumab, timolol, tinzaparin sodium, tipapkinogene sovacivec, tipifarnib, tipiracil hydrochloride, tipranavir, tirapazamine, tirofiban, Tisporin, Tisseel, Tisseel VH, tissue plasminogen activator, tivantinib, TIVb, tizanidine, tobramycin, tocilizumab, tocopherol, tofacitinib, tolvaptan, topiramate, topotecan, topotecan hydrochloride, topsalysin, toralizumab, tositumomab, tovetumab, tPA, trabectedin, trabedersen, trafermin, tralokinumab, tranexamic acid, trastuzumab, trastuzumab biosimilar, trastuzumab emtansine, trebananib, tregalizumab, tremelimumab, trenonacog alpha, treosulfan, tretinoin, trevogrumab, TriAb, triamcinolone, triamcinolone acetate, triamcinolone acetonide, Trichuris suis ova, tridecactide acetate, trifluridine, TriGem, triiodothyronine, trimethoprim, trimetrexate, TriMixDC, Trimovax, tripegfilgrastim, triptorelin, triptorelin acetate, triptorelin pamoate, Tritanrix HB, troglitazone, troplasminogen alpha, TroVax, TRU-015, TRX-318, tucotuzumab celmoleukin, tumor infiltrating lymphocytes, tumor necrosis factor, turoctocog alpha, TXA-127, tyrosinase, tyrosinase 206-214, tyrosinase peptide, Tyrosinase (1-9), Tyrosinase (240-251), Tyrosinase.A2, Tyrosinase/gp100/MART-1 Peptides, ublituximab, UFH, UFT, ularitide, ulinastatin, Ultralente, Unconjugated Myeloma Immunoglobulin plasma paraprotein, unfractionated heparin, urelumab, uric acid, urocortin 3, urocortin II, urofollitropin, urokinase, ursodeoxycholic acid, ustekinumab, Utrogestan, Vacc-4x, Vacc-05, vaccinia-CEA-TRICOM vaccine, vacristine, VAK-694, valaciclovir, valategrast, VALERGEN-BT, VALERGEN-DP, VALERGEN-DS, VALERGENT-BT, valganciclovir, valopicitabine dihydrochloride, valproic acid, valspodar, vancomycin, vaniprevir, vanutide cridificar, vapreotide, Vaqta, varenicline, Varicella live attenuated vaccine with gelatin, Varicella live attenuated vaccine without gelatin, varicella vaccine, varicella-zoster vaccine, VariZIG, Vasoactive Intestinal Peptide, vasopressin, vatalanib, vatelizumab, vatreptacog alpha, Vavelta, Vaxelis, VCL-HB01, VCL-HM01, vecuronium, vedolizumab, vedroprevir, VEGF, VEGFR1-1084, VEGFR2-169, Veglin, velafermin, velaglucerase alpha, Velban, veldoreotide, velimogene aliplasmid, veliparib, velmanase alpha, velpatasvir, veltuzumab, vemurafenib, vepalimomab, Veronate, verteporfin, ViaDor-hPTH, viagenpumatucel-L, Vialebex, vigabatrin, Vigantol, vildagliptin, vinblastine, vinblastine sulphate, vincristin, vincristine, vincristine sulphate, vindesine, vindesine sulphate, vinflunine, vinorelbine, Virulizin, visilizumab, vismodegib, vitespen, Vivaglobin, Vivaglobulin, Vivotif, VM26, VM-26, vobarilizumab, voclosporin, volociximab, Volulyte, Voluven, von Willebrand Factor/Factor VIII, vonapanitase, vonicog alpha, vorhyaluronidase alpha, voriconazole, vorinostat, votucalis, VP16, VR795 varicella Oka strain, W-135, warfarin, WinRho SDF, xenograft, XG-102, XG-104, XL-281, XmAb-5871, Y tetanus toxoid, Ypeginterferon alpha-2b, yttrium Y 90 epratuzumab, yttrium Y 90 monoclonal antibody MN-14, yttrium Y 90 resin microspheres, yttrium-90, Yttrium-90 spheres, yttrium-90-labeled daclizumab, zalcitabine, zalutumumab, zanolimumab, zastumotide, Zemaira, Zevalin, ziconotide, zidovudine, zinc gluconate, ZMapp, zoptarelin doxorubicin, zosuquidar, ZP-Glucagon, ZT-031, and Zutectra.

In some embodiments, a small molecule may be used in lieu of a biological in the above-described list. In some embodiments, an herbal preparation may be used in lieu of a biological in the above-described list. In some embodiments, a borderline product may be used in lieu of a biological in the above-described list.

### Gene Therapy and Cell Therapy

In certain embodiments, a stimulus that perturbs a GSC may be selected from a gene and cell therapy selected from 5-flourouracil, ACT-GRO-777, Ad35-GRIN, adenovirus gene therapy, aganirsen, aglatimagene besadenovec, aglatimagene besadenovec, alferminogene tadenovec, alicaforsen, alicaforsen, anti-HIV ribozyme therapy, anti-PD-1/PD-L1 therapy, aprinocarsen, asvasiran sodium, atesidorsen, ATL-1102, AVI-4126, AVI, baliforsen, beclanorsen, beperminogene perplasmid, bevacizumab, bevasiranib, BioBypass, capecitabine, cenersen, cetuximab, cobitolimod, cyclophosphamide, cystic fibrosis gene therapy, cytarabine, dexamethasone, drisapersen, Electroporation-mediated plasmid DNA vaccine therapy, eteplirsen, fludarabine, G-207 virus construct, ganciclovir, gataparsen, gemcitabine, gemtuzumab ozogamicin, Gendicine, Genedexa, golnerminogene pradenovec, GTU MultiHIV vaccine, HER-2 DNA AutoVac, HIF-1 alpha gene therapy, IFN beta gene therapy, IL-2, cLipid, imetelstat, inotersen, Instiladrin, interferon alfa, IONIS-APO(a)-LRx, irinotecan, ISIS-5132, ISIS-EIF4ERx, itarnafloxin, IT-pIL12-EP, Leuvectin, LIPO-5, LOR-2040, lymphocyte gene therapy, mipomersen, miravirsen, mongersen, MRX-34, MVATG-17401, Mydicar, Nek2 siRNA therapy, Nexagon, nusinersen, oblimersen, ofranergene obadenovec, patisiran, pCF1-CFTR, pexastimogene devacirepvec, pGM169/GL67A gene therapy, ProSavin, ranibizumab, recombinant adeno-associated virus acid alpha-glucosidase, revusiran, Rexin-G, riferminogene pecaplasmid, rituximab, sitimagene ceradenovec, sorafenib, SPK-9001, taberminogene vadenovec, tgAAV-CFTR, VEGF-2 gene therapy, volanesorsen, and ZFP-VEGF.

### Other Treatment Products

In certain embodiments, a stimulus that perturbs a GSC is one of the treatment products selected from 1-propanol, 2-propanol, 5-hydroxytryptophan, abexol, acetaminophen, Acetated Ringer's solution, Acetobacter, acetylcysteine, adenosine, air/oxygen, alanine, alanyl-glutamine, alpha-lipoic acid, Amcrylate, amoxicillin, Antibiophilus, Apevitin BC, AquADEKs-2, armodafinil, Arthronat, AS Orthana, ascorbic acid, aspartate, astaxanthin, Bacillus coagulans GB1-30, Baker's yeast, BeneFlax, benzoyl peroxide, beta-carotene, beta-glucan, bicarbonate, bicarbonate dialysate, BIFICOPEC, Bifidobacterium bifidum BGN4, Bifidobacterium breve M-16V, Bifidobacterium infantis M-63, Bifidobacterium lactis, Bifidobacterium lactis AD011, Bifidobacterium longum BB536, Biograf-G, Bioperine, Biotene, bisacodil, bisacodyl, bismuth, Brassica vegetable, bupropion, caffeine, calcium carbonate, calcium gluconate, calcium polystyrene sulfonate, Carboxymethylcellulose, carnitine complex, carvedilol, Casein, casein phosphopeptide-amorphous calcium phosphate, catechins polyphenols, Cetaphil Dermacontrol Moisturizer SPF-30, chitosan, chloramphenicol, chlorhexidine, chlorhexidine alcohol, chloroquine, cholecalciferol, choline chloride, chondroitin, chondroitin sulphate, cisplatin, citrulline, clarithromycin, CleanDental, ClinOleic, cocamide diethanolamine, cocoa, coenzyme q10, Colocap Balance, Coolprep, copper, corn/soy oil, Cosamin DS, Cremalax, curcumin, cyanoacrylate, cyanocobalamin, cycloserine, D-chiro-inositol, D-cycloserine, Dexeryl, dextrose, DFD-03, DHA, diacerein, diammine silver fluoride, Dibikor, Difinsa53, dimethicome, dipotassium oxalate monohydride, Diprobase, dl-phenylalanine, docosahexaenoic acid, Doublebase, D-serine, Dulcolax, Duraphat, edetate calcium disodium, eformoterol fumarate dihydrate, eicosapentaenoic acid, entecavir, EPA, epicatechin, ethanol, Evonail, Flamazine, Flortec, floxuridine, Flugel FFA, fluoxetine hydrochloride, folate, folic acid, gabapentin, Gastrus, gelatin tannate, gemcitabine, ginger, glucosamine, glucosamine sulphate, glucose, Glucose-potassium-insulin, glucosyl hesperidin, glutathione, glycine, guar gum, Gum Hydral, Hedrin, helium, HeO2, heparin, histidine, Histoacryl, hyaluronic acid, hydrocortisone, hydrogen, hydrogen water, hydrogen-rich dissolution water, Hydromol, ibuprofen, indigocarmine, intralipid, inulin, iodine, iodipin, iron hydroxide polymaltose, isotonic magnesium sulphate, ivermectin, Juven, Lactobacillus acidophilus, Lactobacillus acidophilus AD031, Lactobacillus acidophilus CL-1285, Lactobacillus casei, Lactobacillus rhamnosus, Lactobacillus rhamnosus GG, lactoferrin, lactose, lactulose, LAIS Birch-Alder, lauromacrogol, L-carnosine, lenalidomide, letrozole, leucine rich aminoacid product, leucovorin, levobupivacaine, lidocaine, lipiodol, lipopolysaccharide, Liposyn III, Listerine, L-Menthol, Locapred, Locatop, 1-tyrosine, lutein, magnesium, Magnesium sulphate, maltitol, mannitol, marine n-3 fatty acids, MaxClarity H, melatonin, meloxicam, Melsmon, mesalamine, mesalazine, meso-zeaxanthin, methotrexate, methylphenidate hydrochloride, methylsulfonylmethane, metronidazol, metronidazole, midazolam hydrochloride, minocycline, mitomycin, molecular hydrogen, Moviprep, myo-inositol, Nailprotex, NAVS naphthalan, NeoVIDERM, Neutrafluor 5000, NeutraLice Advance, niacin, Niflec, nitrogen, nitroglycerin, nitrous oxide, Novabone, NuCel, octenidine dihydrochloride, olanzapine, oleanolic acid, oleuropein, Olynth, omega-3 fatty acid, omega-3-carboxylic acids, Omegaven, omeprazole, OPH, Optifast, Optive, Oralgen Pollen, orthosilicic acid, oxygen, Panzytrat ‘HL’, Partly hydrogenated vegetable oils, PB-DM-04, PEG, PEG-400, PEG-Asc, Peglec, phosphatidylcholine, Picosalax, polaprezinc, poly-2-methoxyethylacrylate, polyethylene glycol, potassium chloride, potassium citrate, potassium hydroxide, potassium magnesium citrate, prednisolone, Prevenox, Probiotic B longum, Probiotic S thermophilus, proline, ProTectis, proteoglycan, prucalopride, psilocybin, quercetin, Rafasal, Rennie, resveratrol, Rhinaction, Ringer lactate, Ringerfundin, ropivacaine, rutin, saline, salsalate, sarcosine, Scleremo, serine, sevoflurane, SFCA acetate, SFCA butyrate, SFCA propionate, silver diammine fluoride, simethicone, Sinuclean nebules 45, sodium, sodium alginate, sodium bicarbonate, sodium citrate, sodium cromoglycate, sodium phosphate, sodium polystyrene sulfonate, sodium shale oil sulfonate, soy, soya, stannous fluoride, starch, Sterofundin ISO, Streptococcus salivarius, sulindac, sulphamethoxazole, Systane, Tacholiquine, taurine, tetracycline, Tetraspan, theobromine, theophylline, Trametes versicolor extract, Trans fatty acids, Traumeel, trehalose, triamcinolone acetonide, trichloracetic acid, Triclosan, trimethoprim, Truehope EMP, turmeric, ubiquinone, valerian, Vertise Row, vitamin A palmitate, vitamin B complex, vitamin B12, vitamin B6, isolated whey protein, and zopiclone.

Additional perturbations/drugs which may be utilized include, but are not limited to, TRW3-(2-AMINO-3-HYDROXY-PROPYL)-6-(N′-CYCLOHEXYL-HYDRAZINO)OCTAHYDRO-INDOL-7-OL; ({[(3E)-2′-OXO-2′,7′-DIHYDRO-2,3′-BIINDOL-3(7H)-YLIDENE]AMINO}OXY)ACETIC ACID; 9-Butyl-8-(4-Methoxybenzyl)-9h-Purin-6-Amine; (10R)-10-methyl-3-(6-methylpyridin-3-yl)-9,10,11,12-tetrahydro-8H-[1,4]diazepino[5′,6′:4,5]thieno[3,2-f]quinolin-8-one; (11R)-10-acetyl-11-(2,4-dichlorophenyl)-6-hydroxy-3,3-dimethyl-2,3,4,5,10,11-hexahydro-1H-dibenzo[b,e][1,4]diazepin-1-one; (11S)-8-CHLORO-11-[1-(METHYLSULFONYL)PIPERIDIN-4-YL]-6-PIPERAZIN-1-YL-11H-BENZO[5,6]CYCLOHEPTA[1,2-B]PYRIDINE; 9-HYDROXY-4-PHENYL-6H-PYRROLO[3,4-C]CARBAZOLE-1,3-DIONE; 9-Butyl-8-(3,4,5-Trimethoxybenzyl)-9h-Purin-6-Amine; 9-DEAZAINOSINE-2′,3-O-ETHYLIDENEPHOSPHONATE; AC-(D)PHE-PRO-BOROHOMOORNITHINE-OH; (1R)-N,6-DIHYDROXY-7-METHOXY-2-[(4-METHOXYPHENYL)SULFONYL]-1,2,3,4-TETRAHYDROISOQUINOLINE-1-CARBOXAMIDE; (1R)-1-{[(4′-METHOXY-1,1′-BIPHENYL-4-YL)SULFONYL]AMINO}-2-METHYLPROPYLPHOSPHONIC ACID; (1-Methyl-1h-Imidazol-2-Yl)-(3-Methyl-4-{3-[(Pyridin-3-Ylmethyl)-Amino]-Propoxy}-Benzofuran-2-Yl)-Methanone; Adenosine-5′-[Beta, Gamma-Methylene]Tetraphosphate; Adenosine-5′-Diphosphate-2′,3′-Vanadate; (13S)-13-METHYLDODECAHYDRO-1H-CYCLOPENTA[A]PHENANTHRENE-3,17(2H,4H)-DIONE; (1aR,8S,13S,14S,15aR)-5,13,14-trihydroxy-3-methoxy-8-methyl-8,9,13,14,15,15a-hexahydro-6H-oxireno[k][2]benzoxacyclotetradecine-6,12(1aH)-dione; Adenosine-5′-[Beta, Gamma-Methylene]Triphosphate; ({3-[1-(4-HYDROXY-2-OXO-2H-CHROMEN-3-YL)-PROPYL]-PHENYLCARBAMOYL}-METHYL)-CARBAMIC ACID TERT-BUTYL ESTER; (1R)-2-{[AMINO(IMINO)METHYL]AMINO)-1-(4-[(4R)-4-(HYDROXYMETHYL)-1,3,2-DIOXABOROLAN-2-YL]PHENYL}ETHYL NICOTINATE; Alpha,Alpha,Alpha-Trifluoro-P-Cresol; (1R)-2-PHENYLACETAMIDO-2-(3-CARBOXYPHENYL)ETHYL BORONIC ACID; Alpha-1-proteinase inhibitor; Alpha-Aminobutyric Acid; (1R,2S)-2-(5-thioxo-4,5-dihydro-1H-1,2,4-triazol-3-yl)cyclohexanecarboxylic acid; Adenylyl-3-5′-Phospho-Uridine-3-Monophosphate; Alpha-Cyclodextrin (Cyclohexa-Amylose); (1R,4S,7AS)-1-(1-FORMYLPROP-1-EN-1-YL)-4-METHOXY-2,4,5,6,7,7A-HEXAHYDRO-1H-ISOINDOLE-3-CARBOXYLIC ACID; (1S)-1-(1H-INDOL-3-YLMETHYL)-2-(2-PYRIDIN-4-YL-[1,7]NAPHTYRIDIN-5-YLOXY)-EHYLAMINE; (1S)-1-(3-chlorophenyl)-2-oxo-2-[(1,3,4-trioxo-1,2,3,4-tetrahydroisoquinolin-5-yl)amino]ethyl acetate; Alpha-D-Glucopyranosyl-2-Carboxylic Acid Amide; alpha-D-mannose 6-phosphate; Alpha-D-Galactose-1-Phosphate; Alpha-Difluoromethylornithine; (1S,4R,7AR)-4-BUTOXY-1-[(1R)-1-FORMYLPROPYL]-2,4,5,6,7,7A-HEXAHYDRO-1H-ISOINDOLE-3-CARBOXYLIC ACID; Alpha-Fluoro-Carboxymethyldethia Coenzyme a Complex; (1S)-2-[(2S,5R)-2-(AMINOMETHYL)-5-PROP-1-YN-1-YLPYRROLIDIN-1-YL]-1-CYCLOPENTYL-2-OXOETHANAMINE; ALPHA-HYDROXYFARNESYLPHOSPHONIC ACID; (1S,5S,7R)-N˜7˜-(BIPHENYL-4-YLMETHYL)-N˜3˜HYDROXY-6,8-DIOXA-3-AZABICYCLO[3.2.1]OCTANE-3,7-DICARBOXAMIDE; Alpha-Methylisocitric Acid; (2R)-3-{[(4Z)-5,6-DIPHENYL-6,7-DIHYDRO-4H-PYRROLO[2,3-D]PYRIMIDIN-4-YLIDENE]AMINO}PROPANE-1,2-DIOL; (1S,6R)-3-{[3-(TRIFLUOROMETHYL)-5,6-DIHYDRO[1,2,4]TRIAZOLO[4,3-A]PYRAZIN-7(8H)-YL]CARBONYL}-6-(2,4,5-TRIFLUOROPHENYL)CYCLOHEX-3-EN-1-AMINE; Alpha-Methyl-N-Acetyl-D-Glucosamine; Alpha-N-Dichloroacetyl-P-Aminophenylserinol; (3AR,5R,6S,7R,7AR)-5-(HYDROXYMETHYL)-2-PROPYL-5,6,7,7A-TETRAHYDRO-3AH-PYRANO[3,2-D][1,3]THIAZOLE-6,7-DIOL; Alpha-Ribazole-5′-Phosphate; (3ASR,4RS,8ASR,8BRS)-4-(2-(4-FLUOROBENZYL)-1,3-DIOXODEACAHYDROPYRROLO[3,4-A] PYRROLIZIN-4-YL)BENZAMIDINE; (3aS)-3a-hydroxy-5-methyl-1-phenyl-1,2,3,3a-tetrahydro-4H-pyrrolo[2,3-b]quinolin-4-one; Alpha-Ribazole-5′-Phosphate Derivative; (3AS,4R,9BR)-2,2-DIFLUORO-4-(4-HYDROXYPHENYL)-1,2,3,3A,4,9B-HEXAHYDROCYCLOPENTA[C]CHROMEN-8-OL; ANDROSTA-1,4-DIENE-3,17-DIONE; (3AS,4R,9BR)-4-(4-HYDROXYPHENYL)-1,2,3,3A,4,9B-HEXAHYDROCYCLOPENTA[C]CHROMEN-8-OL; Anthrax immune globulin human; autologous activated macrophage therapy; (3a5)-3a-hydroxy-1-phenyl-1,2,3,3a-tetrahydro-4H-pyrrolo[2,3-b]quinolin-4-one; (1-Tert-Butyl-5-Hydroxy-1h-Pyrazol-4-Yl)-(6-Methanesulfonyl-4′-Methoxy-2-Methyl-Biphenyl-3-Yl)-Methanone; Autologous cultured chondrocytes; (1S,3R,6S)-4-oxo-6-{4-[(2-phenylquinolin-4-yl)methoxy]phenyl}-5-azaspiro[2.4]heptane-1-carboxylic acid; Beta-1,2,3,4,6-Penta-O-Galloyl-D-Glucopyranose; (1S)-2-(1H-INDOL-3-YL)-1-{[(5-ISOQUINOLIN-6-YLPYRIDIN-3-YL)OXY]METHYL}ETHYLAMINE; (2-{[(4-BROMO-2-FLUOROBENZYL)AMINO]CARBONYL}-5-CHLOROPHENOXY)ACETIC ACID; Beta-D-Arabinofuranose-5′-Phosphate; (20S)-19,20,22,23-TETRAHYDRO-19-OXO-5H,21H-18,20-ETHANO-12,14-ETHENO-6,10-METHENOBENZ[D]IMIDAZO[4,3-L][1,6,9,13]OXATRIAZACYCLONOADECOSINE-9-CARBONITRILE; beta-D-galactopyranosyl-(1-&gt;4)-beta-D-galactopyranose; (1S,2S,3R,4S,5S)-2,3,4-TRIHYDROXY-5-(HYDROXYMETHYL)CYCLOHEXYL (1E)-2-PHENYL-N-(SULFOOXY)ETHANIMIDOTHIOATE; Beta-D-Glucopyranose Spirohydantoin; (2R)-1-[4-({4-[(2,5-DICHLOROPHENYL)AMINO]PYRIMIDIN-2-YL}AMINO)PHENOXY]-3-(DIMETHYLAMINO)PROPAN-2-OL; (2R)-1-[(5,6-DIPHENYL-7H-PYRROLO[2,3-D]PYRIMIDIN-4-YL)AMINO]PROPAN-2-OL; (2R)-1-[4-({6-[(2,6-DIFLUOROPHENYL)AMINO]PYRIMIDIN-4-YL}AMINO)PHENOXY]-3-(DIMETHYLAMINO)PROPAN-2-OL; bis(4-hydroxyphenyl)methanone; (2R)-1-(DIMETHYLAMINO)-3-{4-[(6-{[2-FLUORO-5-(TRIFLUOROMETHYL)PHENYL]AMINO}PYRIMIDIN-4-YL)AMINO]PHENOXY}PROPAN-2-OL; Biliverdin 1× Gamma Chromophore; (2r)-2-(Aminomethyl)-2,4-Dihydroxy-5-Oxo-3-(2-Oxoethyl)-2,5-Dihydro-1h-Imidazol-3-lum; Bis(5-Amidino-2-Benzimidazolyl)Methane Ketone; Bis(5-Amidino-Benzimidazolyl)Methane; (2R)-2-AMINO-3,3,3-TRIFLUORO-N-HYDROXY-2-{[(4-PHENOXYPHENYL)SULFONYL]METHYL}PROPANAMIDE; Bis(Adenosine)-5-Pentaphosphate; (2R)-3-{[(BENZYLAMINO)CARBONYL]AMINO}-2-HYDROXYPROPANOIC ACID; (2R)-4-[(8R)-8-METHYL-2-(TRIFLUOROMETHYL)-5,6-DIHYDRO[1,2,4]TRIAZOLO[1,5-A]PYRAZIN-7(8H)-YL]-4-OXO-1-(2,4,5-TRIFLUOROPHENYL)BUTAN-2-AMINE; (2S)-2-[3-(AMINOMETHYL)PHENYL]-3-{(S)-HYDROXY[(1R)-2-METHYL-1-{[(2-PHENYLETHYL)SULFONYL]AMINO}PROPYL]PHOSPHORYL}PROPANOIC ACID; (2R)-4-(2-BENZOYL-1,2-DIAZEPAN-1-YL)-4-OXO-1-(2,4,5-TRIFLUOROPHENYL)BUTAN-2-AMINE; Bis(Adenosine)-5′-Triphosphate; (2R,3R)-3-{[3,5-BIS(TRIFLUOROMETHYL)PHENYL]AMINO}-2-CYANO-3-THIOXOPROPANAMIDE; BOC-GAMMA-D-GLU-L-LYS(CBZ)-D-BOROALA; (2S)-2-[3-(AMINOMETHYL)PHENYL]-3-[(R)-HYDROXY{(1R)-2-METHYL-1-[(PHENYLSULFONYL)AMINO]PROPYL}PHOSPHORYL]PROPANOIC ACID; (2S)-4-(2,5-DIFLUOROPHENYL)-N,N-DIMETHYL-2-PHENYL-2,5-DIHYDRO-1H-PYRROLE-1-CARBOXAMIDE; (2S)-4-(2,5-difluorophenyl)-N-[(3R,4S)-3-fluoro-1-methylpiperidin-4-yl]-2-(hydroxymethyl)-N-methyl-2-phenyl-2,5-dihydro-1H-pyrrole-1-carboxamide; C1 Esterase Inhibitor (Recombinant); C-(1-Hydrogyl-Beta-D-Glucopyranosyl) Formamide; C1 Esterase Inhibitor (Human); (1S)-2-oxo-1-phenyl-2-[(1,3,4-trioxo-1,2,3,4-tetrahydroisoquinolin-5-yl)amino]ethyl acetate; Carba-Nicotinamide-Adenine-Dinucleotide; (2S)-2-(3-{[AMINO(IMINO)METHYL]AMINO}PHENYL)-3-[(S)-HYDROXY(3-PHENYLPROPYL)PHOSPHORYL]PROPANOIC ACID; (2R,3R)-7-(methylsulfonyl)-3-(2,4,5-trifluorophenyl)-1,2,3,4-tetrahydropyrido[1,2-a]benzimidazol-2-amine; (2R,3R)-N\hat{ }1\hat{ }-[(1S)-2,2-DIMETHYL-1-(METHYLCARBAMOYL)PROPYL]-N\hat{ }4\hat{ }-HYDROXY-2-(2-METHYLPROPYL)-3-{[(1,3-THIAZOL-2-YLCARBONYL)AMINO]METHYL}BUTANEDIAMIDE; (2r,3r,4r,5r)-3,4-Dihydroxy-N,N′-Bis[(1s,2r)-2-Hydroxy-2,3-Dihydro-1h-Inden-1-Yl]-2,5-Bis(2-Phenylethyl)Hexanediamide; CHLORAMPHENICOL SUCCINATE; (2S)-1-(6H-INDOL-3-YL)-3-{[5-(7H-PYRAZOLO[3,4-C]PYRIDIN-5-YL)PYRIDIN-3-YL]OXY}PROPAN-2-AMINE; (2R)—N-HYDROXY-2-[(3S)-3-METHYL-3-{4-[(2-METHYLQUINOLIN-4-YL)METHOXY]PHENYL}-2-OXOPYRROLIDIN-1-YL]PROPANAMIDE; (2R,4R)-N˜1˜-(4-CHLOROPHENYL)-N˜2˜-[2-FLUORO-4-(2-OXOPYRIDIN-1 (2H)-YL)PHENYL]-4-METHOXYPYRROLIDINE-1,2-DICARBOXAMIDE; Chorionic Gonadotropin (Recombinant); (2r)-N-[4-Cyano-3-(Trifluoromethyl)Phenyl]-3-[(4-Fluorophenyl)Sulfonyl]-2-Hydroxy-2-Methylpropanamide; Coagulation Factor IX (Recombinant); Cmp-2-Keto-3-Deoxy-Octulosonic Acid; (2S)-1-(1H-INDOL-3-YL)-3-{[5-(3-METHYL-1H-INDAZOL-5-YL)PYRIDIN-3-YL]OXY}PROPAN-2-AMINE; (2E)-3-(3,4-DIHYDROXYPHENYL)-N-[2-(4-HYDROXYPHENYL)ETHYL]ACRYLAMIDE; (2S)-1-[4-({4-[(2,5-DICHLOROPHENYL)AMINO]PYRIMIDIN-2-YL}AMINO)PHENOXY]-3-(DIMETHYLAMINO)PROPAN-2-OL; CCR5 mAb (human monoclonal antibody to CCR5); Coa-S-Acetyl 5-Bromotryptamine; Collagenase Clostridium histolyticum; (2R,3R,4R,5S)-2-(HYDROXYMETHYL)-1-NONYLPIPERIDINE-3,4,5-TRIOL; (2R,3R,4S,5R)-2-[6-amino-8-[(3,4-dichlorophenyl)methylamino]purin-9-yl]-5-(hydroxymethyl)oxolane-3,4-diol; cyclic 3′,5′-thymidine monophosphate; (2S)-1-{[5-(1H-INDAZOL-5-YL)PYRIDIN-3-YL]OXY}-3-[(7AS)-7AH-INDOL-3-YL]PROPAN-2-AMINE; (2-CARBAMOYLMETHYL-5-PROPYL-OCTAHYDRO-INDOL-7-YL)ACETIC ACID; D-[(N-Hydroxyamino)Carbonyl]Phenylalanine; (2S)-2-({6-[(3-amino-5-chlorophenyl)amino]-9-isopropyl-9H-purin-2-yl}amino)-3-methylbutan-1-ol; Decylamine-N,N-Dimethyl-N-Oxide; D-(L-a-Aminoadipoyl)-L-Cysteinyl-D-Isodehydrovaline; (2S)-2-(4-CHLOROPHENYL)-2-[4-(1H-PYRAZOL-4-YL)PHENYL]ETHANAMINE; (2E)-1-[2-hydroxy-4-methoxy-5-(3-methylbut-2-en-1-yl)phenyl]-3-(4-hydroxyphenyl)prop-2-en-1-one; Delta-Amino Valeric Acid; (2S)-1-{4-[(4-ANILINO-5-BROMOPYRIMIDIN-2-YL)AMINO]PHENOXY}-3-(DIMETHYLAMINO)PROPAN-2-OL; Delta-Bis(2,2′-Bipyridine)Imidazole Ruthenium (Ii); (2s)-2-[(2,4-Dichloro-Benzoyl)-(3-Trifluoromethyl-Benzyl)-Amino]-3-Phenyl-Propionic Acid; (2S)-2-[(3aR,4R,7S,7aS)-1,3-dioxooctahydro-2H-4,7-methanoisoindol-2-yl]propanoic acid; (2s)-2-[(5-Benzofuran-2-Yl-Thiophen-2-Ylmethyl)-(2,4-Dichloro-Benzoyl)-Amino]-3-Phenyl-Propionic Acid; (2S)-2-{[3-(3-aminophenyl)imidazo[1,2-b]pyridazin-6-yl]amino}-3-methylbutan-1-ol; BIS(HEXAMETHYLENE)TRIAMINE; (2S)-5-hydroxy-2-(4-hydroxyphenyl)-7-methoxy-2,3-dihydro-4H-chromen-4-one; (2S,3S)-3-{3-[2-chloro-4-(methylsulfonyl)phenyl]-1,2,4-oxadiazol-5-yl}-1-cyclopentylidene-4-cyclopropyl-1-fluorobutan-2-amine; DIETHYL 4-METHOXYPHENYL PHOSPHATE; (2S,3S)-3-AMINO-4-(3,3-DIFLUOROPYRROLIDIN-1-YL)-N,N-DIMETHYL-4-OXO-2-(TRANS-4-[1,2,4]TRIAZOLO[1,5-A]PYRIDIN-6-YLCYCLOHEXYL)BUTANAMIDE; D-leucyl-N-(4-carbamimidoylbenzyl)-L-prolinamide; (2S,3S,4R,5R,6R)-5-Amino-2-(Aminomethyl)-6-((2R,3R,4R,5S)-5-((1R,2R,3S,5R,6S)-3,5-Diamino-2-((2S,3R,4R,5S,6R)-3-amino-4,5-dihydroxy-6-(hydroxymethyl)-tetrahydro-2H-pyran-2-yloxy)-6-hydroxycyclohexyloxy)-2-(hydroxymethyl)-4-(2-((R)-piperidin-3-ylmethylamin; DIETHYL PROPANE-1,3-DIYLBISCARBAMATE; D-phenylalanyl-N-{4-[amino(iminio)methyl]benzyl}-L-prolinamide; D-Myo-Inositol-1,4-Bisphosphate; (3-ENDO)-8-METHYL-8-AZABICYCLO[3.2.1]OCT-3-YL 1H-PYRROLO[2,3-B]PYRIDINE-3-CARBOXYLATE; (3-EXO)-3-(10,11-DIHYDRO-5H-DIBENZO[A,D][7]ANNULEN-5-YLOXY)-8,8-DIMETHYL-8-AZONIABICYCLO[3.2.1]OCTANE; D-Myo-Inositol-2,4,5-Trisphosphate; (4-AMINO-2-{[1-(METHYLSULFONYL)PIPERIDIN-4-YL]AMINO}PYRIMIDIN-5-YL)(2,3-DIFLUORO-6-METHOXYPHENYL)METHANONE; D-Naphthyl-1-Acetamido Boronic Acid Alanine; (3R)-3-(aminomethyl)-9-methoxy-1,2,3,4-tetrahydro-5H-[1]benzothieno[3,2-e][1,4]diazepin-5-one; ETHYL (1E)-2-PHENYL-N-(SULFOOXY)ETHANIMIDOTHIOATE; Flavin adenine dinucleotide; (3R)-3-[(1,2,3,4-tetrahydroisoquinolin-7-yloxy)methyl]-2,3-dihydrothieno[2,3-f][1,4]oxazepin-5-amine; D-phenylalanyl-N-(3-fluorobenzyl)-L-prolinamide; (2S)-3-(1-{[2-(2-CHLOROPHENYL)-5-METHYL-1,3-OXAZOL-4-YL]METHYL}-1H-INDOL-5-YL)-2-ETHOXYPROPANOIC ACID; Di-Stearoyl-3-Sn-Phosphatidylethanolamine; (2S,3S)-3-AMINO-4-[(3S)-3-FLUOROPYRROLIDIN-1-YL]-N,N-DIMETHYL-4-OXO-2-(TRANS-4-[1,2,4]TRIAZOLO[1,5-A]PYRIDIN-5-YLCYCLOHEXYL)BUTANAMIDE; (2S,3S)-3-FORMYL-2-({[(4-METHYLPHENYL)SULFONYL]AMINO}METHYL)PENTANOIC ACID; (2S)-1-AMINO-3-[(5-NITROQUINOLIN-8-YL)AMINO]PROPAN-2-OL; (2S,3S)-4-cyclopropyl-3-{(3R,5R)-3-[2-fluoro-4-(methylsulfonyl)phenyl]-1,2,4-oxadiazolidin-5-yl}-1-[(3S)-3-fluoropyrrolidin- 1-yl]-1-oxobutan-2-amine; (2S)-6-(2,4-DIAMINO-6-ETHYLPYRIMIDIN-5-YL)-2-(3,5-DIFLUOROPHENYL)-4-(3-METHOXYPROPYL)-2H-1,4-BENZOXAZIN-3(4H)-ONE; ETHYL 3-[4-(AMINOSULFONYL)PHENYL]PROPANOATE; Flavin-Adenine Dinucleotide-N5-Isobutyl Ketone; (2S)-2-{[HYDROXY(4-IODOBENZYL)PHOSPHORYL]METHYL}PENTANEDIOIC ACID; (2S)-2-{3-[({[2-fluoro-4-(trifluoromethyl)phenyl]carbonyl}amino)methyl]-4-methoxybenzyl}butanoic acid; Glycyl-L-a-Aminopimelyl-E-(D-2-Aminoethyl)Phosphonate; (2S)N-ACETYL-L-ALANYL-ALPHAL-PHENYLALANYL-CHLOROETHYLKETONE; Glycyl-L-Alpha-Amino-Epsilon-Pimelyl-D-Alanine; (2R,3R,4S)-3-(4-HYDROXYPHENYL)-4-METHYL-2-[4-(2-PYRROLIDIN-1-YLETHOXY)PHENYL]CHROMAN-6-OL; GUANOSINE-2′,3′-O-ETHYLIDENEPHOSPHONATE; (2R)-N-hydroxy-3-naphthalen-2-yl-2-[(naphthalen-2-ylsulfonyl)amino]propanamide; (1S,3aS,5aR,8aS)-1,7,7-trimethyl-1,2,3,3a,5a,6,7,8-octahydrocyclopenta[c]pentalene-4-carboxylic acid; (2e)-N-Alyl-4-{[3-(4-Bromophenyl)-5-Fluoro-1-Methyl-1h-Indazol-6-Yl]Oxy}-N-Methyl-2-Buten-1-Amine; (2E)-N-hydroxy-3-[1-methyl-4-(phenylacetyl)-1H-pyrrol-2-yl]prop-2-enamide; (1S,2R,3S,4R,5R)-2,3,4-trihydroxy-N-octyl-6-oxa-8-azabicyclo[3.2.1]octane-8-carbothioamide; HGS-TR2J (human monoclonal antibody to TRAIL Receptor 2); (2s,3s,8s,9s)-3-Amino-9-Methoxy-2,6,8-Trimethyl-10-Phenyldeca-4,6-Dienoic Acid; (2E,4E)-11-METHOXY-3,7,11-TRIMETHYLDODECA-2,4-DIENOIC ACID; (2S)-8-[(tert-butoxycarbonyl)amino]-2-(1H-indol-3-yl)octanoic acid; (2S)—N-(4-cyano-3-iodophenyl)-3-(4-cyanophenoxy)-2-hydroxy-2-methylpropanamide; (1R)-1-(2-thienylacetylamino)-1-phenylmethylboronic acid; L-Alfa-Lysophosphatidylcholine, Lauroyl; (1,10 Phenanthroline)-(Th-Carbon Monoxide) Rhenium (1); (16ALPHA,17ALPHA)-ESTRA-1,3,5(10)-TRIENE-3,16,17-TRIOL; (1H-indol-3-yl)-(2-mercapto-ethoxyimino)-acetic acid; (1-HYDROXYDODECANE-1,1-DIYL)BIS(PHOSPHONIC ACID); Lambda-Bis(2,2′-Bipyridine)Imidazole Ruthenium (Ii); (1-HYDROXYNONANE-1,1-DIYL)BIS(PHOSPHONIC ACID); 3-(5-amino-3-imino-3H-pyrazol-4-ylazo)-benzoic acid; (1R)-1,2,2-TRIMETHYLPROPYL (R)-METHYLPHOSPHINATE; (1R)-4-(3-phenoxyphenyl)-1-phosphonobutane-1-sulfonic acid; (1R)-MENTHYL HEXYL PHOSPHONATE GROUP; (1R, 2S)-cis 1,2 dihydroxy-1,2-dihydronaphthalene; (1S,2S,3R,6R)-4-(hydroxymethyl)-6-(octylamino)cyclohex-4-ene-1,2,3-triol; Methoxy arachidonyl fluorophosphonate; (1S,2R,3S,4R,5S)-8-AZABICYCLO[3.2.1]OCTANE-1,2,3,4-TETROL; (1S)-MENTHYL HEXYL PHOSPHONATE GROUP; (1S,2S,5S)2-(4-GLUTARIDYLBENZYL)-5-PHENYL-1-CYCLOHEXANOL; (1S,4R,9S)-5-(trifluoromethyl)-1,2,3,4-tetrahydro-1,4-methanonaphthalen-9-amine; (1S,4S)-4-(3,4-dichlorophenyl)-N-methyl-1,2,3,4-tetrahydronaphthalen-1-amine; (1Z)-2-HYDROXY-3-OXOHEX-1-EN-1-YL DIHYDROGEN PHOSPHATE; (1Z)-4-(4-FLUOROPHENYL)-2-METHYLIDENEBUTAN-1-IMINE; (2,2-DIPHOSPHONOETHYL)(DODECYL)DIMETHYLPHOSPHONIUM; (2-ACETYL-5-METHYLANILINO)(2,6-DIBROMOPHENYL)ACETAMIDE; (2-AMINO-1,3-OXAZOL-5-YL)-(3-BROMOPHENYL)METHANONE; (2-AMINO-3-PHENYL-BICYCLO[2.2.1]HEPT-2-YL)-PHENYL-METHANONE; (2-BROMOETHYL)(2-′FORMYL-4′-AMINOPHENYL) ACETATE; (2E)-3-(2,4-DICHLOROPHENYL)-N-HYDROXYACRYLAMIDE; (2E)-3-(2-OCT-1-YN-1-YLPHENYL)ACRYLIC ACID; (2E)-3-(3,4-DIHYDROXYPHENYL)-2-IMINOPROPANOIC ACID; N-(2,6-Diflouro-Benzyl)-4-Sulfamoyl-Benzamide; (2E)-3-(4-CHLOROPHENYL)-N-HYDROXYACRYLAMIDE; (2E,4E)-2-HYDROXY-6-OXO-6-PHENYLHEXA-2,4-DIENOIC ACID; (2E,4R,5S)-2,3,4,5-TETRAHYDROXY-6-(PALMITOYLOXY)HEX-2-ENOIC ACID; (2R)-1-[(4-tert-butylphenyl)sulfonyl]-2-methyl-4-(4-nitrophenyl)piperazine; N-(1-benzylpiperidin-4-yl)-4-sulfanylbutanamide; (2R)-2-({9-(1-methylethyl)-6-[(4-pyridin-2-ylbenzyl)amino]-9H-purin-2-yl}amino)butan-1-ol; (2R)-2-(4-CHLOROPHENYL)-2-PHENYLETHANAMINE; N-(2-Acetamido)Iminodiacetic Acid; (2R)-3-(phosphonooxy)propane-1,2-diyl diheptanoate; (2R)-4,4-dihydroxy-5-nitro-2-(phenylmethyl)pentanoic acid; (2r)-Amino(3,5-Dihydroxyphenyl)Acetic Acid; (2R)-amino(4-hydroxyphenyl)ethanoic acid; (2E)-3-(3-hydroxy-4-methoxyphenyl)prop-2-enoic acid; (2r,4s)-2-Methyl-2,3,3,4-Tetrahydroxytetrahydrofuran; (2S)-1-(2,5-dimethylphenoxy)-3-morpholin-4-ylpropan-2-ol; (2S)-1-(9H-Carbazol-4-yloxy)-3-(isopropylamino)propan-2-ol; N-(3,5-dibromo-4-hydroxyphenyl)benzamide; (2S)-1-methyl-2-[(2S,4R)-2-methyl-4-phenylpentyl]piperidine; N-(3-AMINOPROPYL)-2-NITROBENZENAMINE; (2S)-2-AMINO-1-(5-TERT-BUTYL-1,3,4-OXADIAZOL-2-YL)PROPAN-1-ONE; (2S)-2-AMINO-4-(METHYLSULFANYL)-1-(1,3-THIAZOL-2-YL)BUTANE-1,1-DIOL; (2s)-2-Amino-4-(Methylsulfanyl)-1-Pyridin-2-Ylbutane-1,1-Diol; (2S)-2-amino-5-oxo-5-[(4-phenylmethoxyphenyl)amino]pentanoic acid; (2S)-2-HYDROXY-2H-CHROMENE-2-CARBOXYLICACID; N-(3-MERCAPTOPROPANOYL)-D-ALANINE; (2S)-2-hydroxy-2-methyl-N-[4-nitro-3-(trifluoromethyl)phenyl]-3-(pentafluorophenoxy)propanamide; (2S)-2-methyl-2,3-dihydrothieno[2,3-f][1,4]oxazepin-5-amine; (2S)-3-(4-chloro-3-fluorophenoxy)-N-[4-cyano-3-(trifluoromethyl)phenyl]-2-hydroxy-2-methylpropanamide; (2S)-3-[(9H-fluoren-9-ylideneamino)oxy]-2-methylpropanoic acid; (2S)-3-[(R)-[(1S)-1-amino-3-phenylpropyl](hydroxy)phosphoryl]-2-benzylpropanoic acid; (2S)-3-[4-(acetylamino)phenoxy]-2-hydroxy-2-methyl-N-[4-nitro-3-(trifluoromethyl)phenyl]propanamide; (2S)-4-(4-fluorobenzyl)-N-(2-sulfanylethyl)piperazine-2-carboxamide; (2S)-4-(4-fluorobenzyl)-N-(3-sulfanylpropyl)piperazine-2-carboxamide; (2s)-4-(Beta-Alanylamino)-2-Aminobutanoic Acid; (2s,3r)-3-Amino-2-Hydroxy-5-(Ethylsulfanyl)Pentanoyl-((S)-(−)-(1-Naphthyl)Ethyl)Amide; (2S,3S)-3-(4-fluorophenyl)-2,3-dihydroxypropanoic acid; (2z)-2-(Benzoylamino)-3-[4-(2-Bromophenoxy)Phenyl]-2-Propenoic Acid; (2s,3s)-Trans-2,3-Dihydro-3-Hydroxyanthranilic Acid; (2Z)-2-cyano-N-(2,2′-dichlorobiphenyl-4-yl)-3-hydroxybut-2-enamide; (2Z)-2-cyano-N-(3′-ethoxybiphenyl-4-yl)-3-hydroxybut-2-enamide; (2z)-3-{[Oxido(Oxo)Phosphino]Oxy}-2-Phenylacrylate; (2Z)-5-BROMO-2,3′-BIINDOLE-2′,3(1H,1′H)-DIONE AMMONIATE; (2Z)-N-(3-chloro-2′-methoxybiphenyl-4-yl)-2-cyano-3-hydroxybut-2-enamide; (2Z)-N-biphenyl-4-yl-2-cyano-3-cyclopropyl-3-hydroxyprop-2-enamide; N-(5-(4-CHLORO-3-(2-HYDROXY-ETHYLSULFAMOYL)-PHENYLTHIAZOLE-2-YL)-ACETAMIDE; (2Z,3E)-2,3′-BIINDOLE-2′,3(1H,1′H)-DIONE 3-{O-[(3R)-3,4-DIHYDROXYBUTYL]OXIME}; N-(4-MORPHOLINE)CARBONYL-B-(1-NAPHTHYL)-L-ALANINE-L-LEUCINE BORONIC ACID; (2Z,4E)-3-chloro-2-hydroxy-6-oxo-6-phenylhexa-2,4-dienoic acid; (3R)-8-(dioxidosulfanyl)-3-methyl-1,2,3,4-tetrahydroquinoline; (3,3-dimethylpiperidin-1-yl)(6-(3-fluoro-4-methylphenyl)pyridin-2-yl)methanone; (3,4,5-Trihydroxy-6-Hydroxymethyl-Tetrahydro-Pyran-2-Yl)-Phosphoramidic Acid Dimethyl Ester; (3,4-DIHYDROXY-2-NITROPHENYL)(PHENYL)METHANONE; (3,4-Dihydroxy-Phenyl)-Triphenyl-Arsonium; (3-{3-[[2-Chloro-3-(Trifluoromethyl)Benzyl](2,2-Diphenylethyl)Amino]Propoxy}Phenyl)Acetic Acid; (3-Carboxy-2-(R)-Hydroxy-Propyl)-Trimethyl-Ammonium; N-(5-{[(2S)-4-amino-2-(3-chlorophenyl)butanoyl]amino}-1H-indazol-3-yl)benzamide; (3E)-3-[(phenylamino)methylidene]dihydrofuran-2(3H)-one; (3R)-3-ethyl-N-[(4-methylphenyl)sulfonyl]-L-aspartic acid; (3E)-4-(1-METHYL-1H-INDOL-3-YL)BUT-3-EN-2-ONE; N-(5-Amino-5-Carboxypentyl)Glutamic Acid; (3E)-5-fluoro-1-[(6-fluoro-4H-1,3-benzodioxin-8-yl)methyl]-1H-indole-2,3-dione 3-oxime; (3e)-6′-Bromo-2,3′-Biindole-2′,3(1h,1′h)-Dione 3-Oxime; (3r)-3-{[(Benzyloxy)Carbonyl]Amino}- 2-Oxo-4-Phenylbutane-1-Diazonium; (3R)-3-cyclopentyl-6-methyl-7-[(4-methylpiperazin-1-yl)sulfonyl]-3,4-dihydro-2H-1,2-benzothiazine 1,1-dioxide; N-(5′-Phosphopyridoxyl)-D-Alanine; N-(ethoxycarbonyl)-L-leucine; (3r)-4-(P-Toluenesulfonyl)-1,4-Thiazane-3-Carboxylicacid-L-Leucine; N-(Chlorophenyl)-N′-Hydroxyguanidine; (3R)-4,4-DIFLUORO-3-[(4-METHOXYPHENYL)SULFONYL]BUTANOIC ACID; (3R)-4-[(3R)-3-AMINO-4-(2,4,5-TRIFLUOROPHENYL)BUTANOYL]-3-(2,2,2-TRIFLUOROETHYL)-1,4-DIAZEPAN-2-ONE; N-(Phosphonoacetyl)-L-Ornithine; (3R)-4-{[(3,4-dihydroxyphenyl)acetyl]oxy}-N-(2-formylindolizin-3-yl)-3-sulfino-D-valine; (3R)-METHYLCARBAMOYL-7-SULFOAMINO-3,4-DIHYDRO-1H-ISOQUINOLINE-2-CARBOXYLIC ACID BENZYL ESTER; N-(QUINOLIN-8-YL)METHANESULFONAMIDE; (3R)-N-methyl-3-phenyl-3-[4-(trifluoromethyl)phenoxy]propan-1-amine; (3R,4R)-1-{6-[3-(METHYLSULFONYL)PHENYL]PYRIMIDIN-4-YL}-4-(2,4,5-TRIFLUOROPHENYL)PIPERIDIN-3-AMINE; (3R,4R)-4-(pyrrolidin-1-ylcarbonyl)-1-(quinoxalin-2-ylcarbonyl)pyrrolidin-3-amine; (3R,4R,5R)-5-(HYDROXYMETHYL)-1-(3-PHENYLPROPYL)PIPERIDINE-3,4-DIOL; (3r,4r,5r)-5-(Hydroxymethyl)Piperidine-3,4-Diol; (3R,4S)-1-(3,4-DIMETHOXYPHENYL)-3-(3-METHYLPHENYL)PIPERIDIN-4-AMINE; (3R,4S)-1-[(4-amino-5H-pyrrolo[3,2-d]pyrimidin-7-yl)methyl]-4-[(benzylsulfanyl)methyl]pyrrolidin-3-ol; (3R,4S)-1-[(4-amino-5H-pyrrolo[3,2-d]pyrimidin-7-yl)methyl]-4-[(butylsulfanyl)methyl]pyrrolidin-3-ol; N-(Sulfanylacetyl)Tyrosylprolylmethioninamide; (3R,4S)-1-[6-(6-METHOXYPYRIDIN-3-YL)PYRIMIDIN-4-YL]-4-(2,4,5-TRIFLUOROPHENYL)PYRROLIDIN-3-AMINE; N-[1-(2,6-dimethoxybenzyl)piperidin-4-yl]-4-sulfanylbutanamide; N-[12-(1H-imidazol-1-yl)dodecanoyl]-L-leucine; (3r,5r)-7-((1r,2r,6s,8r,8as)-2,6-Dimethyl-8-{[(2r)-2-Methylbutanoyl]Oxy}-1,2,6,7,8,8a-Hexahydronaphthalen-1-Yl)-3,5-Dihydroxyheptanoic Acid; (3R,5S,7R,12S,13R)-13-FORMYL-12,14-DIHYDROXY-3,5,7-TRIMETHYLTETRADECANOIC ACID; [1H-INDOL-3-YL-ACETYL]ASPARTIC ACID; (3S)-1-(1,3-BENZODIOXOL-5-YLMETHYL)-3-[4-(1H-IMIDAZOL-1-YL)PHENOXY]PIPERIDINE; (3S)-1-(2-hydroxyphenyl)-5-oxopyrrolidine-3-carboxylic acid; (3S)-1-(4-acetylphenyl)-5-oxopyrrolidine-3-carboxylic acid; (3S)-1-{[4-(but-2-yn-1-yloxy)phenyl]sulfonyl}pyrrolidine-3-thiol; (3S)-1-CYCLOHEXYL-5-OXO-N-PHENYLPYRROLIDINE-3-CARBOXAMIDE; N-[4-(2,4-Dimethyl-1,3-Thiazol-5-Yl)Pyrimidin-2-Yl]-N′-Hydroxyimidoformamide; (3s)-3,4-Di-N-Hexanoyloxybutyl-1-Phosphocholine; (3s)-3-Amino-1-(Cyclopropylamino)Heptane-2,2-Diol; (3S)-3-cyclopentyl-6-methyl-7-[(4-methylpiperazin-1-yl)sulfonyl]-3,4-dihydro-2H-1,2,4-benzothiadiazine 1,1-dioxide; (3S)-3-hydroxy-1-methyl-2,3-dihydro-1H-indole-5,6-dione; (3S)-4-{[4-(BUT-2-YNYLOXY)PHENYL]SULFONYL}-N-HYDROXY-2,2-DIMETHYLTHIOMORPHOLINE-3-CARBOXAMIDE; (3S)—N-(3-BROMOPHENYL)-1-CYCLOHEXYL-5-OXOPYRROLIDINE-3-CARBOXAMIDE; N-[4-(3-BROMO-PHENYLAMINO)-QUINAZOLIN-6-YL]-ACRYLAMIDE; (3S)—N-(5-CHLORO-2-METHYLPHENYL)-1-CYCLOHEXYL-5-OXOPYRROLIDINE-3-CARBOXAMIDE; (3S)-N-methyl-3-phenyl-3-[4-(trifluoromethyl)phenoxy]propan-1-amine; N-[N-[1-Hydroxycarboxyethyl-Carbonyl]Leucylamino-Butyl]-Guanidine; (3S,5E)-3-propyl-3,4-dihydrothieno[2,3-f][1,4]oxazepin-5(2H)-imine; (3S,5R,7R,8S,9S,10R)-7-(hydroxymethyl)-3-(2-naphthyl)-1,6-dioxa-2-azaspiro[4.5]decane-8,9,10-triol; N-[4-Hydroxymethyl-Cyclohexan-6-Yl-1,2,3-Triol]-4,6-Dideoxy-4-Aminoglucopyranoside; (3S,6S)-3,6-bis(4-hydroxybenzyl)piperazine-2,5-dione; (3s,6s,9r,10r,11s,12s,13e,15e,18s,21s)-18-{(1e,3e,7s,8s)-9-[(2s,3r,4s,5s,6r,9s,11s)-9-Ethyl-4-Hydroxy-3,5,11-Trimethyl-8-Oxo-1-Oxa-7-Azaspiro[5.5]Undec-2-Yl]-8-Hydroxy-1,7-Dimethylnona-1,3-Dienyl}-10,12-Dihydroxy-3-(3-Hydroxybenzyl)-6-Isopropyl-11-Methyl-; (3s,8ar)-3-(1h-Imidazol-5-Ylmethyl)Hexahydropyrrolo[1,2-a]Pyrazine-1,4-Dione; N-[0-Phosphono-Pyridoxyl]-Isoleucine; (3Z)-1-[(6-fluoro-4H-1,3-benzodioxin-8-yl)methyl]-4-[(E)-2-phenylethenyl]-1H-indole-2,3-dione 3-oxime; (3Z)-1-[(6-fluoro-4H-1,3-benzodioxin-8-yl)methyl]-4-phenyl-1H-indole-2,3-dione 3-oxime; (3Z)-6-(4-HYDROXY-3-METHOXYPHENYL)-3-(1H-PYRROL-2-YLMETHYLENE)-1,3-DIHYDRO-2H-INDOL-2-ONE; (3Z)-N,N-DIMETHYL-2-OXO-3-(4,5,6,7-TETRAHYDRO-1H-INDOL-2-YLMETHYLIDENE)-2,3-DIHYDRO-1H-INDOLE-5-SULFONAMIDE; N˜3˜-[3-(5-METHOXYPYRIDIN-3-YL)BENZYL]PYRIDINE-2,3-DIAMINE; (3Z,5S,6R,7S,8R,8aS)-3-(octylimino)hexahydro[1,3]thiazolo[3,4-a]pyridine-5,6,7,8-tetrol; N-cycloheptylglycyl-N-(4-carbamimidoylbenzyl)-L-prolinamide; (4-{(2S)-2-[(tert-butoxycarbonyl)amino]-3-methoxy-3-oxopropyl}phenyl)methaneseleninic acid; (4′-{[Allyl(Methyl)Amino]Methyl}-1,1′-Biphenyl-4-Yl)(4-Bromophenyl)Methanone; (4-{2-Acetylamino-2-[1-(3-Carbamoyl-4-Cyclohexylmethoxy-Phenyl)-Ethylcarbamoyl}-Ethyl}-2-Phosphono-Phenoxy)-Acetic Acid; (4aS)-5-[(2,4-diaminopteridin-6-yl)methyl]-4a,5-dihydro-2H-dibenzo[b,f]azepin-8-ol; N-cyclopropyl-4-pyrazolo[1,5-b]pyridazin-3-ylpyrimidin-2-amine; (4-BROMOPHENYL)[4-({(2E)-4-[CYCLOPROPYL(METHYL)AMINO]BUT-2-ENYL}OXY)PHENYL]METHANONE; (4E)-N-(4-fluorophenyl)-4-[(phenylcarbonyl)imino]-4H-pyrazole-3-carboxamide; (4e,8e,12z,16z)-N,N,4,8,13,17,21-Heptamethyldocosa-4,8,12,16,20-Pentaen-1-Amine; (4R)-4-(3-butoxy-4-methoxybenzyl)imidazolidin-2-one; (4R)-4-(3-HYDROXYPHENYL)-N, N,7,8-TETRAMETHYL-3,4-DIHYDROISOQUINOLINE-2(1H)-CARBOXAMIDE; N-ethyl-N-[3-(propylamino)propyl]propane-1,3-diamine; (4R)-7-chloro-9-methyl-1-oxo-1,2,4,9-tetrahydrospiro[beta-carboline-3,4′-piperidine]-4-carbonitrile; (3s)-Tetrahydrofuran-3-YI (1r,2s)-3-[4-((1r)-2-{[(S)- Amino(Hydroxy)Methyl]Oxy}-2,3-Dihydro-1h-Inden-1-Yl)-2-Benzyl-3-Oxopyrrolidin-2-Yl]-1-Benzyl-2-Hydroxypropylcarbamate; Nicotinamide Adenine Dinucleotide 3-Pentanone Adduct; (4R,5R)-5-AMINO-1-[2-(1,3-BENZODIOXOL-5-YL)ETHYL]-4-(2,4,5-TRIFLUOROPHENYL)PIPERIDIN-2-ONE; (4r,5s,6s,7r)-1,3-Dibenzyl-4,7-Bis(Phenoxymethyl)-5,6-Dihydroxy-1,3 Diazepan-2-One; (4s)-2-[(1e)-1-Aminoprop-1-Enyl]-4,5-Dihydro-1,3-Thiazole-4-Carboxylic Acid; (4S)-4-(2-NAPHTHYLMETHYL)-D-GLUTAMIC ACID; (4s)-4-{[(2s)-2-Amino-3-Oxopropyl]Sulfanyl}-L-Homoserinate; (4S,5E,7Z,10Z,13Z,16Z,19Z)-4-hydroxydocosa-5,7,10,13,16,19-hexaenoic acid; (4s-Trans)-4-(Methylamino)-5,6-Dihydro-6-Methyl-4h-Thieno(2,3-B)Thiopyran-2-Sulfonamide-7,7-Dioxide; (4-sulfamoyl-phenyl)-thiocarbamic acid 0-(2-thiophen-3-yl-ethyl) ester; Nicotinamide Adenine Dinucleotide Acetone Adduct; (4Z)-6-bromo-4-({[4-(pyrrolidin-1-ylmethyl)phenyl]amino}methylidene)isoquinoline-1,3(2H,4H)-dione; N-Pyridoxyl-1-Amino-Cyclopropanecarboxylic Acid-5-Monophosphate; (5-(PYRIDIN-3-YL)FURAN-2-YL)METHANAMINE; (5,6-DIPHENYL-FURO[2,3-D]PYRIMIDIN-4-YLAMINO)-ACETIC; N-Naphthalen-1-Ylmethyl-2′-[3,5-Dimethoxybenzamido]-2′-Deoxy-Adenosine; (5-BROMO-4-CHLORO-3-INDOLYL)-A-D-MANNOSE; (5-CHLORO-2-{[(3-NITROBENZYL)AMINO]CARBONYL}PHENOXY)ACETIC ACID; (5-Chloropyrazolo[1,5-a]Pyrimidin-7-Yl)-(4-Methanesulfonylphenyl)Amine; Open Form of 2′-Deoxy-Ribofuranose-5′-Phosphate; (5E)-14-CHLORO-15,17-DIHYDROXY-4,7,8,9,10,11-HEXAHYDRO-2-BENZOXACYCLOPENTADECINE-1,12(3H,13H)-DIONE; (5E)-5-[(2,2-DIFLUORO-1,3-BENZODIOXOL-5-YL)METHYLENE]-1,3-THIAZOLIDINE-2,4-DIONE; (5E,13E)-11-HYDROXY-9,15-DIOXOPROSTA-5,13-DIEN-1-OIC ACID; (5e,13e)-9,15-Dihydroxy-11-Oxoprosta-5,13-Dien-1-Oicacid; (5E,14E)-11-oxoprosta-5,9,12,14-tetraen-1-oic acid; (5E,7S)-2-amino-7-(4-fluoro-2-pyridin-3-ylphenyl)-4-methyl-7,8-dihydroquinazolin-5(6H)-one oxime; (5-Methyl-6-Oxo-1,6-Dihydro-Pyridin-3-Yl)-1,2-Dideoxy-Ribofuranose-5-Monophosphate; (5-Oxo-5,6-Dihydro-Indolo[1,2-a]Quinazolin-7-Yl)-Acetic Acid; (5-phenyl-7-(pyridin-3-ylmethylamino)pyrazolo[1,5-a]pyrimidin-3-yl)methanol; (5R)-2-[(2-fluorophenyl)amino]-5-(1-methylethyl)-1,3-thiazol-4(5H)-one; (5R)-2-SULFANYL-5-[4-(TRIFLUOROMETHYL)BENZYL]-1,3-THIAZOL-4-ONE; PARA-NITROBENZYL GLUTARYL GLYCINIC ACID; P1-(5′-Adenosyl)P5-(5-(3azido-3′-Deoxythymidyl))Pentaphosphate; (5R)-5-[(1E)-BUTA-1,3-DIENYL]-4-HYDROXY-3,5-DIMETHYLTHIOPHEN-2(5H)-ONE; (5R,6E,8Z,11Z,14Z,17Z)-5-hydroxyicosa-6,8,11,14,17-pentaenoic acid; (5r)-6-(4-{[2-(3-Iodobenzyl)-3-Oxocyclohex-1-En-1-Yl]Amino}Phenyl)-5-Methyl-4,5-Dihydropyridazin-3(2h)-One; Phosphomethylphosphonic Acid Guanylate Ester; Phosphoaminophosphonic Acid Guanylate Ester; Phosphomethylphosphonic Acid Adenosyl Ester; (5S)-2-{[(1S)-1-(4-fluorophenyl)ethyl]amino}-5-(1-hydroxy-1-methylethyl)-5-methyl-1,3-thiazol-4(5H)-one; (5R,7R,8S,9S,10R)-7-(hydroxymethyl)-3-phenyl-1,6-dioxa-2-azaspiro[4.5]dec-2-ene-8,9,10-triol; (5S)-1-benzyl-3-(1,1-dioxido-1,2-benzisothiazol-3-yl)-4-hydroxy-5-(1-methylethyl)-1,5-dihydro-2H-pyrrol-2-one; (5S)-2-(cyclooctylamino)-5-methyl-5-propyl-1,3-thiazol-4(5H)-one; Phosphomethylphosphonic Acid-Guanylate Ester; (5R,6S,8S)-8-[3-(AMINOMETHYL)PHENYL]-6-HYDROXY-5-ISOPROPYL-3-OXO-1-PHENYL-2,7-DIOXA-4-AZA-6-PHOSPHANONAN-9-OIC ACID 6-OXIDE; (5S)-3-ANILINO-5-(2,4-DIFLUOROPHENYL)-5-METHYL-1,3-OXAZOLIDINE-2,4-DIONE; (5S)-4,5-difluoro-6-[(2-fluoro-4-iodophenyl)imino]-N-(2-hydroxyethoxy)cyclohexa-1,3-diene-1-carboxamide; Phosphothiophosphoric Acid-Adenylate Ester; (5S)-5-(3-AMINOPROPYL)-3-(2,5-DIFLUOROPHENYL)-N-ETHYL-5-PHENYL-4,5-DIHYDRO-1H-PYRAZOLE-1-CARBOXAMIDE; (5s)-5-Iododihydro-2,4(1h,3h)-Pyrimidinedione; (5S,6S)-6-[(R)ACETOXYETH-2-YL]-PENEM-3-CARBOXYLATEPROPANE; (5S,8R,9S,10S,13R,14S,17S)-13-{2-[(3,5-DIFLUOROBENZYL)OXY]ETHYL}-17-HYDROXY-10-METHYLHEXADECAHYDRO-3H-CYCLOPENTA[A]PHENANTHREN-3-ONE; PUROMYCIN AMINONUCLEOSIDE-5′-MONOPHOSPHATE; rhMBL (recombinant human mannose binding lectin); (5z)-2-[(1s,2r)-1-Amino-2-Hydroxypropyl]-5-[(4-Amino-1h-Indol-3-Yl)Methylene]-3-(2-Hydroxyethyl)-3,5-Dihydro-4h-Imidazol-4-One; (5Z)-3-(4-CHLOROPHENYL)-4-HYDROXY-5-(1-NAPHTHYLMETHYLENE)FURAN-2(5H)-ONE; (5z)-5-(1h-Indol-3-Ylmethylene)-4h-Imidazol-4-One; (5Z)-5-(3-BROMOCYCLOHEXA-2,5-DIEN-1-YLIDENE)-N-(PYRIDIN-4-YLMETHYL)-1,5-DIHYDROPYRAZOLO[1,5-A]PYRIMIDIN-7-AMINE; (6,7-Difluoro-Quinazolin-4-Yl)-(1-Methyl-2,2-Diphenyl-Ethyl)-Amine; S-Ethyl-N-[4-(Trifluoromethyl)Phenyl]Isothiourea; (6,7-Dihydro-5h-Cyclopenta[D]Imidazo[2,1-B]Thiazol-2-Yl]-4,7-Dihydro[1,4]Thiazepine-3,6-Dicarboxylic Acid; (6-[4-(AMINOMETHYL)-2,6-DIMETHYLPHENOXY]-2-{[4-(AMINOMETHYL)PHENYL]AMINO}-5-BROMOPYRIMIDIN-4-YL)M ETHANOL; Sodium phosphate, monobasic, monohydrate; (6e)-6-[(2e,4e,6e)-3,7-Dimethylnona-2,4,6,8-Tetraenylidene]-1,5,5-Trimethylcyclohexene; Sodium phosphate, monobasic, anhydrous; (6E,11E)-HEPTADECA-6,11-DIENE-9,9-DIYLBIS(PHOSPHONIC ACID); Technetium Tc 99m nofetumomab merpentan; (6R)-2-amino-6-[2-(3-methoxybiphenyl-3-yl)ethyl]-3,6-dimethyl-5,6-dihydropyrimidin-4(3H)-one; UNDECA-3,7-DIENE-1,3,7,11-TETRACARBALDEHYDE; (6S)-1-chloro-3-[(4-fluorobenzyl)oxy]-6-(pyrrolidin-1-ylcarbonyl)pyrrolo[1,2-a]pyrazin-4(6H)-one; (6S)-2-amino-6-(3′-methoxybiphenyl-3-yl)-3,6-dimethyl-5,6-dihydropyrimidin-4(3H)-one; (6S)-6-CYCLOPENTYL-6-[2-(3-FLUORO-4-ISOPROPOXYPHENYL)ETHYL]-4-HYDROXY-5,6-DIHYDRO-2H-PYRAN-2-ONE; (7as,12ar,12bs)-1,2,3,4,7a,12,12a,12b-Octahydroindolo[2,3-a]Quinolizin-7(6h)-One; (7R,8R)-8-(2,4,5-trifluorophenyl)-6,7,8,9-tetrahydroimidazo[1,2-a:4,5-c]dipyridin-7-amine; (7S)-2-(2-aminopyrimidin-4-yl)-7-(2-fluoroethyl)-1,5,6,7-tetrahydro-4H-pyrrolo[3,2-c]pyridin-4-one; (8alpha,10alpha,13alpha,17beta)-17-[(4-hydroxyphenyl)carbonyl]androsta-3,5-diene-3-carboxylic acid; Uridine-5′-Diphosphate-N-Acetylmuramoyl-L-Alanine; (8R,9Z,12Z)-8-hydroxy-6-oxooctadeca-9,12-dienoic acid; (9ALPHA,13BETA,17BETA)-2-[(1Z)-BUT-1-EN-1-YL]ESTRA-1,3,5(10)-TRIENE-3,17-DIOL; (9aS)-4-bromo-9a-butyl-7-hydroxy-1,2,9,9a-tetrahydro-3H-fluoren-3-one; (9BETA,11ALPHA,13ALPHA,14BETA,17ALPHA)-11-(METHOXYMETHYL)ESTRA-1(10),2,4-TRIENE-3,17-DIOL; (9BETA,13ALPHA,14BETA,17ALPHA)-2-METHOXYESTRA-1,3,5(10)-TRIENE-3,17-DIYL DISULFAMATE; (9r,10r)-9-(S-Glutathionyl)-10-Hydroxy-9,10-Dihydrophenanthrene; (9S)-9-[(8-AMMONIOOCTYL)AMINO]-1,2,3,4,9,10-HEXAHYDROACRIDINIUM; (9S, 10E,12Z)-9-hydroxyoctadeca-10,12-dienoic acid; (9s,10s)-9-(S-Glutathionyl)-10-Hydroxy-9,10-Dihydrophenanthrene; (9S, 12S)-9-(1-methylethyl)-7,10-dioxo-2-oxa-8, 11-diazabicyclo[12.2.2]octadeca-1(16),14, 17-triene-12-carboxylic acid; (CHLOROACETYL)CARBAMIC ACID (3R,4S,5S,5R)-5-M ETHOXY-4-[(2R,3R)-2-METHYL-3-(3-METHYL-2-BUTENYL)OXIRANYL]-1-OXASPIRO[2.5]OCT-6-YL ESTER; 4-[5-(4-FLUORO-PHENYL)-2-(4-METHANESULFINYL-PHENYL)-3H-IMIDAZOL-4-YL]-PYRIDINE; (E)-(2r,3r,4s,5r)-3,4,5-Trihydroxy-2-Methoxy-8,8-Dimethyl-Non-6-Enoic Acid ((3s,6r)-6-Hydroxy-2-Oxo-Azepan-3-Yl)-Amide; (E)-(4S,6S)-6-((S)-2-{(S)-2-[(FURAN-2-CARBONYL)-AMINO]-3-METHYL-BUTYRYLAMINO}-3-METHYL-BUTYRYLAMINO)-8-METHYL-5-OXO-4-((R)-2-OXO-PYRROLIDIN-3-YLMETHYL)-NON-2-ENOIC ACID ETHYL ESTER; 4-BROMO-3-(CARBOXYMETHOXY)-5-[3-(CYCLOHEXYLAMINO)PHENYL]THIOPHENE-2-CARBOXYLIC ACID; (E)-(S)-4-[(S)-2-((S)-2-TERT-BUTOXYCARBONYLAMINO-3-METHYL-BUTYRYLAMINO)-2-PHENYL-ACETYLAMINO]-5-(2-OXO-PYRROLIDIN-3-YL)-PENT-2-ENOIC ACID ETHYL ESTER; 4-[(5-{[4-(3-CHLOROPHENYL)-3-OXOPIPERAZIN-1-YL]METHYL}-1H-IMIDAZOL-1-YL)METHYL]BENZONITRILE; (E)-[4-(3,5-difluorophenyl)-3H-pyrrolo[2,3-b]pyridin-3-ylidene](3-methoxyphenyl)methanol; 4-{[(2,6-dichlorophenyl)carbonyl]amino}-N-piperidin-4-yl-1H-pyrazole-3-carboxamide; 4-{2-(4-Fluoro-Benzyl)-6-Methyl-5-[(5-Methyl-Isoxazole-3-Carbonyl)-Amino]-4-Oxo-Heptanoylamino}-5-(2-Oxo-Pyrrolidin-3-Yl)-Pentanoic Acid Ethyl Ester; (METHYLPYRIDAZINE PIPERIDINE BUTYLOXYPHENYL)ETHYLACETATE; (METHYLPYRIDAZINE PIPERIDINE ETHYLOXYPHENYL)ETHYLACETATE; (METHYLPYRIDAZINE PIPERIDINE PROPYLOXYPHENYL)ETHYLACETATE; (N-{4-[(ETHYLANILINO)SULFONYL]-2-METHYLPHENYL}-3,3,3-TRI FLUORO-2-HYDROXY-2-METHYLPROPANAMIDE; (R)-(+) 5(9BH)-OXO-9B-PHENYL-2,3-DIHYDROTHIAZOLO[2,3-A]ISOINDOL-3-CARBOXYLIC ACID METHYL ESTER; (R)-(+)9B-(3-METHYL)PHENYL-2,3-DIHYDROTHIAZOLO[2,3-A]ISOINDOL-5(9BH)-ONE; 4-{[(1R,2S)-1,2-dihydroxy-2-methyl-3-(4-nitrophenoxy)propyl]amino}-2-(trifluoromethyl)benzonitrile; (R)-1-(4-(4-(HYDROXYMETHYL)-1,3,2-DIOXABOROLAN-2-YL)PHENETHYL)GUANIDINE; 4-{5-[(1Z)-1-(2-IMINO-4-OXO-1,3-THIAZOLIDIN-5-YLIDENE)ETHYL]-2-FURYL}BENZENESULFONAMIDE; (R)-2-(FORMYLOXY)-3-(PHOSPHONOOXY)PROPYL PENTANOATE; (R)-3-BROMO-2-HYDROXY-2-METHYL-N-[4-NITRO-3-(TRIFLUOROMETHYL)PHENYL]PROPANAMIDE; (R)â€N[2-[1-(aminoiminomethyl)-3-piperidinyl]-1-oxoethyl]-4-(phenylethynyl)-1-phenylalanine methylester; (R)-TRANS-4-(1-AMINOETHYL)-N-(4-PYRIDYL) CYCLOHEXANECARBOXAMIDE; 4-Benzoylamino-4-{1-{1-Carbamoyl-2-[4-(Difluoro-Phosphono-Methyl)-Phenyl]-Ethylcarbamoyl}-2-[4-(Difluoro-Phosphono-Methyl)-Phenyl]-Ethylcarbamoyl}-Butyric Acid; (RP,SP)-O-(2R)-(1-PHENOXYBUT-2-YL)-METHYLPHOSPHONIC ACID CHLORIDE; (S)-(+)-2-[4-(FLUOROBENZYLOXY-BENZYLAMINO)PROPIONAMIDE]; 4-{[(2-OXO-1,2-DIHYDRO-3H-INDOL-3-YLIDENE)METHYL]AMINO}-N-(1,3-THIAZOL-2-YL)BENZENESULFONAMIDE; (S)-2-((S)-3-ISOBUTYL-2,5-DIOXO-4-QUINOLIN-3-YLMETHYL-[1,4]DIAZEPAN-1YL)-N-METHYL-3-NAPHTALEN-2-YL-PROPIONAMIDE; (S)-2-(MERCAPTOMETHYL)-5-PHENYLPENTANOIC ACID; (S)-2(Phosphonoxy)Caproyl-L-Leucyl-P-Nitroanilide; 4-(4-METHYLPIPERAZIN-1-YL)-N-[5-(2-THIENYLACETYL)-1,5-DIHYDROPYRROLO[3,4-C]PYRAZOL-3-YL]BENZAMIDE; (S)-2-[4-(Aminomethyl)-1h-1,2,3-Triazol-1-Yl]-4-Methylpentanoic Acid; (5)-2-{Methyl-[2-(Naphthalene-2-Sulfonylamino)-5-(Naphthalene-2-Sulfonyloxy)-Benzoyl]-Amino}-Succinicacid; (S)-2-CHLORO-N-(1-(2-(2-HYDROXYETHYLAMINO)-2-OXOETHYL)-2-OXO-1,2,3,4-TETRAHYDROQUINOLIN-3-YL)-6H-THIENO[2,3-B]PYRROLE-5-CARBOXAMIDE; 4-[4-(2,3-DIHYDRO-1,4-BENZODIOXIN-6-YL)-3-METHYL-1H-PYRAZOL-5-YL]-6-ETHYLBENZENE-1,3-DIOL; (S)-2-Amino-3-(6h-Selenolo[2,3-B]-Pyrrol-4-Yl)-Propionic Acid; (S)-2-Amino-4-[(2s,3r)-2,3,5-Trihydroxy-4-Oxo-Pentyl]Mercapto-Butyric Acid; (S)-2-Amino-3-(1,3,5,7-Pentahydro-2,4-Dioxo-Cyclopenta[E]Pyrimidin-1-Yl) Proionic Acid; (S)-2-METHYL-1-[(4-METHYL-5-ISOQUINOLINE)SULFONYL]-HOMOPIPERAZINE; 4-[4-(2,5-DIOXO-PYRROLIDIN-1-YL)-PHENYLAMINO]-4-HYDROXY-BUTYRIC ACID; (S)-4-Bromo-3-Hydroxy-3-Methylbutyl Diphosphate; (S)-4-ISOPROPOXYCARBONYL-6-METHOXY-3-METHYLTHIOMETHYL-3,4-DIHYDROQUINOXALIN-2(1H)-THIONE; (S)-5-(4-Benzyloxy-Phenyl)-4-(7-Phenyl-Heptanoylamino)-Pentanoic Acid; 4-HYDROXYBENZALDEHYDE 0-(3,3-DIMETHYLBUTANOYL)OXIME; (S)—N-(3-Indol-1-Yl-2-Methyl-Propyl)-4-Sulfamoyl-Benzamide; (S)—N-(4-carbamimidoylbenzyl)-1-(2-(cyclohexylamino)ethanoyl)pyrrolidine-2-carboxamide; (S)—N-(4-carbamimidoylbenzyl)-1-(2-(cyclopentylamino)ethanoyl)pyrrolidine-2-carboxamide; (S)—N-(4-carbamimidoylbenzyl)-1-(2-(cyclohexyloxy)ethanoyl)pyrrolidine-2-carboxamide; (S)—N-(4-carbamimidoylbenzyl)-1-(2-(cyclopentyloxy)ethanoyl)pyrrolidine-2-carboxamide; (S)—N-(4-carbamimidoylbenzyl)-1-(3-cyclohexylpropanoyl)pyrrolidine-2-carboxamide; (S)—N-(4-carbamimidoylbenzyl)-1-(3-cyclopentylpropanoyl)pyrrolidine-2-carboxamide; (S)-TETRAHYDROFURAN-3-YL (2S,3S)-4-((S)-4-((1R,3R)-3-(2-AMINO-2-OXOETHYL)-2,3-DIHYDRO-1H-INDEN-1-YL)-2-BENZYL-3-OXO-2,3-DIHYDRO-1H-PYRROL-2-YL)-3-HYDROXY-1-PHENYLBUTAN-2-YLCARBAMATE; (TERT-BUTYLOXYCARBONYL)-ALANYL-AMINO ETHYL-FORMAMIDE; (Z)-3-BENZYL-5-(2-HYDROXY-3-NITROBENZYLIDENE)-2-THIOXOTHIAZOLIDIN-4-ONE; (Z,Z)-4-Hydroxy-N,N,N-Trimethyl-10-Oxo-7-[(1-Oxo-9-Octadecenyl)Oxy]-3,5,9-Trioxa-4-Phosphaheptacos-18-En-1-Aminium-4-Oxide; [(1-{2[(4-Carbamimidoyl-Phenylamino)-Methyl]-1-Methyl-1h-Benzoimidazol-5-Yl}-Cyclopropyl)-Pyridin-2-Yl-Methyleneaminooxy]-Acetic Acid Ethyl Ester; 4-chloro-N-(3-methoxypropyl)-N-[(3S)-1-(2-phenylethyl)piperidin-3-yl]benzamide; [(2-AMINO-ALPHA-METHOXYIMINO-4-THIAZOLYLACETYL)AMINO]METHYLBORONIC ACID; (S)-3-(4-(2-Carbazol-9-Yl-Ethoxy)-Phenyl)-2-Ethoxy-Propionic Acid; [(2r,3s,4r,5r)-5-(6-Amino-9h-Purin-9-Yl)-3,4-Dihydroxytetrahydro-2-Furanyl]Methyl Sulfamate; [(2r,3s,4s,5r)-3,4,5-Trihydroxytetrahydrofuran-2-Yl]Methyl Dihydrogen Phosphate; [(2S)-4-methyl-3-oxo-2,3,4,5-tetrahydro-1H-1,4-benzodiazepin-2-yl]acetic acid; [(3R,4S)-4-HYDROXY-3-METHYL-2-OXOHEXYL]PHOSPHONIC ACID; [(3R)-7-NITRO-1,2,3,4-TETRAHYDROISOQUINOLIN-3-YL]METHANOL; [(3,7,11-TRIMETHYL-DODECA-2,6,10-TRIENYLOXYCARBAMOYL)-METHYL]-PHOSPHONIC ACID; [(3R,4S,5S,7R)-4,8-DIHYDROXY-3,5,7-TRIMETHYL-2-OXOOCTYL]PHOSPHONIC ACID; [(3S)-9-hydroxy-1-methyl-10-oxo-4,10-dihydro-3H-benzo[g]isochromen-3-yl]acetic acid; [(4-{4-[4-(Difluoro-Phosphono-Methyl)-Phenyl]-Butyl}-Phenyl)-Difluoro-Methyl]-Phosphonic Acid; [(4R)-2,2-DIMETHYL-1,3-DIOXOLAN-4-YL]METHYL HYDROGEN HEX-5-ENYLPHOSPHONATE; [(4R)-4-(3-HYDROXYPHENYL)-1,6-DIMETHYL-2-THIOXO-1,2,3,4-TETRAHYDROPYRIMIDIN-5-YL]PHENYL)METHANONE; 4-Carbamoyl-4-{[6-(Difluoro-Phosphono-Methyl)-Naphthalene-2-Carbonyl]-Amino}-Butyric Acid; [(5R)-5-(2,3-dibromo-5-ethoxy-4-hydroxybenzyl)-4-oxo-2-thioxo-1,3-thiazolidin-3-yl]acetic acid; [[(3R,4R,5S,6R)-3-(butanoylamino)-4,5-dihydroxy-6-(hydroxymethyl)oxan-2-ylidene]amino] N-phenylcarbamate; [[(3R,4R,5S,6R)-4,5-dihydroxy-6-(hydroxymethyl)-3-(pentanoylamino)oxan-2-ylidene]amino] N-phenylcarbamate; [[1-[N-HYDROXY-ACETAMIDYL]-3-METHYL-BUTYL]-CARBONYL-LEUCINYL]-ALANINE ETHYL ESTER; [[CYCLOHEXANESULFONYL-GLYCYL]-3[PYRIDIN-4-YL-AMINOMETHYL]ALANYL]PIPERIDINE; [{2-bromo-4-[(2R)-3-oxo-2,3-diphenylpropyl]phenyl}(difluoro)methyl]phosphonic acid; 5-AMINO-3-{[4-(AMINOSULFONYL)PHENYL]AMINO}-N-(2,6-DIFLUOROPHENYL)-1H-1,2,4-TRIAZOLE-1-CARBOTHIOAMIDE; [1(1-Methyl-4,5-Dioxo-Pent-2-Enylcarbamoyl)-2-Phenyl-Ethyl]-Carbamic Acid Benzyl Ester; [1-(3-CHLORO-2-FORMYL-PHENYLCARBAMOYL)-2-METHYL-PROPYL]-CARBAMIC ACID TERT-BUTYL ESTER; [1-(3-Hydroxy-2-Oxo-1-Phenethyl-Propylcarbamoyl)2-Phenyl-Ethyl]-Carbamic Acid Pyridin-4-Ylmethyl Ester; [1-(4-Fluorobenzyl)Cyclobutyl]Methyl (1s)-1-[Oxo(1h-Pyrazol-5-Ylamino)Acetyl]Pentylcarbamate; 5-[3-(BENZYLAMINO)PHENYL]-4-BROMO-3-(CARBOXYMETHOXY)THIOPHENE-2-CARBOXYLIC ACID; [1-HYDROXY-2-(1,1′:3′,1″-TERPHENYL-3-YLOXY)ETHANE-1,1-DIYL]BIS(PHOSPHONIC ACID); [2-(1-Amino-2-Hydroxy-Propyl)-4-(1h-Indol-3-Ylmethylene)-5-Oxo-4,5-Dihydro-Imidazol-1-Yl]-Acetaldehyde; [1-(1-Benzyl-3-Hydroxy-2-Oxo-Propylcarbamoyl)-2-Phenyl-Ethyl]-Carbamic Acid Benzyl Ester; [2-(3-DIBENZOFURAN-4-YL-PHENYL)-1-HYDROXY-1-PHOSPHONO-ETHYL]-PHOSPHONIC ACID; [2-(5-MERCAPTO-[1,3,4]THIADIAZOL-2-YLCARBAMOYL)-1-PHENYL-ETHYL]-CARBAMIC ACID BENZYL ESTER; [2-(Methyleneamine)-4-(4-Hydroxy-Benzylidine)-5-Oxo-4,5-Dihydro-Imidazol-1-Yl]-Acetaldehyde; [2,4,6-Triisopropyl-Phenylsulfonyl-L-[3-Amidino-Phenylalanine]]-Piperazine-N′-Beta-Alanine; [2-Amino-6-(2,6-Difluoro-Benzoyl)-Imidazo[1,2-a]Pyridin-3-Yl]-Phenyl-Methanone; [2-Aminomethyl-5-Oxo-4-(4-Oxo-Cyclohexa-2,5-Dienylmethyl)-4,5-Dihydro-Imidazol-1-Yl]-Acetaldehyde; [2′-CARBOXYLETHYL]-10-METHYL-ANTHRACENE ENDOPEROXIDE; 5-[4-(DIMETHYLAMINO)PHENYL]-6-[(6-MORPHOLIN-4-YLPYRIDIN-3-YL)ETHYNYL]PYRIMIDIN-4-AMINE; [4-({4-[(5-CYCLOPROPYL-1H-PYRAZOL-3-YL)AMINO]QUINAZOLIN-2-YL}IMINO)CYCLOHEXA-2,5-DIEN-1-YL]ACETONITRILE; [3-(1,3,2-Dioxaborolan-2-Yloxy)Propyl]Guanidine; [3-(1-Benzyl-3-Carbamoylmethyl-2-Methyl-1h-Indol-5-Yloxy)-Propyl-]-Phosphonic Acid; [3-(4-Bromo-2-Fluoro-Benzyl)-7-Chloro-2,4-Dioxo-3,4-Dihydro-2h-Quinazolin-1-Yl]-Acetic Acid; [3-(Dodecanoylamino)Propyl](Hydroxy)Dimethylammonium; [3,5-Dibromo-4-(4-Hydroxy-3-Phenethylcarbamoyl-Phenoxy)-Phenyl]-Acetic Acid; [4-({[5-Benzyloxy-1-(3-Carbamimidoyl-Benzyl)-1h-Indole-2-Carbonyl]-Amino}-Methyl)-Phenyl]-Trimethyl-Ammonium; 5-Chloro-1h-Indole-2-Carboxylic Acid{[Cyclopentyl-(2-Hydroxy-Ethyl)-Carbamoyl]-Methyl}-Amide; [4-({4-[(5-cyclopropyl-1H-pyrazol-3-yl)amino]quinazolin-2-yl}amino)phenyl]acetonitrile; [2′-HYDROXY-3-(1H-PYRROLO[3,2-C]PYRIDIN-2-YL)-BIPHENYL-3-YLMETHYL]-UREA; [4-({5-(AMINOCARBONYL)-4-[(3-METHYLPHENYL)AMINO]PYRIMIDIN-2-YL}AMINO)PHENYL]ACETIC ACID; 5-(4′-AMINO-1′-ETHYL-5′,8′-DIFLUORO-1′H-SPIRO[PIPERIDINE-4,2′-QUINAZOLINE]-1-YLCARBONYL)PICOLINONITRILE; [4-(2-Amino-4-Methyl-Thiazol-5-Yl)-Pyrimidin-2-Yl]-(3-Nitro-Phenyl)-Amine; [4-(3-AMINOMETHYL-PHENYL)-PIPERIDIN-1-YL]-(5-PHENETHYL-PYRIDIN-3-YL)-METHANONE; [4-(4-ACETYLAMINO-PHENYL)-3,5-DIOXO-4-AZA-TRICYCLO[5.2.2.0 2,6]UNDEC-1-YLCARBAMOYLOXY]-ACETIC ACID; [4-(4-Hydroxy-3-Iodo-Phenoxy)-3,5-Diiodo-Phenyl]-Acetic Acid; 5-CHLORO-N-{(3S)-1-[(1S)-1-METHYL-2-MORPHOLIN-4-YL-2-5-CHLORO-N-{(3S)-1-[(1S)-1-METHYL-2-MORPHOLIN-4-YL-2-SULFONAMIDE; [4-(4-PHENYL-PIPERIDIN-1-YL)-BENZENESULFONYLAMINO]-ACETIC ACID; 5-chloro-N-({(5S)-2-oxo-3-[4-(3-oxomorpholin-4-yl)phenyl]-1,3-oxazolidin-5-yl}methyl)thiophene-2-carboxamide; [5-AMINO-1-(4-FLUOROPHENYL)-1H-PYRAZOL-4-Yl](3-{[(2R)-2,3-DIHYDROXYPROPYL]OXY}PHENYL)METHANONE; [4,6-O-(1-CARBOXYETHYLIDENE)-BETA-D-MANNOSE]; [4-amino-2-(tert-butylamino)-1,3-thiazol-5-yl](phenyl)methanone; [4-R-(4-ALPHA,6-BETA,7-BETA]-HEXAHYDRO-5,6-DI(HYDROXY)-1,3-DI(ALLYL)-4,7-BISPHENYLMETHYL)-2H-1,3-DIAZEPINONE; 5-FLUORO-1-[4-(4-PHENYL-3,6-DIHYDROPYRIDIN-1(2H)-YL)BUTYL]QUINAZOLINE-2,4(1H,3H)-DIONE; [4-(6-Chloro-Naphthalene-2-Sulfonyl)-Piperazin-1-Yl]-(3,4,5,6-Tetrahydro-2h-[1,4′]Bipyridinyl-4-Yl)-Methanone; [5-AMINO-1-(4-FLUOROPHENYL)-1H-PYRAZOL-4-YL][3-(PIPERIDIN-4-YLOXY)PHENYL]METHANONE; [5-HYDROXY-2-(4-HYDROXYPHENYL)-1-BENZOFURAN-7-YL]ACETONITRILE; [5-hydroxy-6-methyl-4-({[(4E)-3-oxo-1,2-oxazolidin-4-ylidene]amino}methyl)pyridin-3-yl]methyl dihydrogen phosphate; [Cyclohexylethyl]-[[[[4-[2-Methyl-1-Imidazolyl-Buty]Phenyl]Acetyl]-Seryl]-Lysinyl]-Amine; 5-(4-PHENOXYPHENYL)-5-(4-PYRIMIDIN-2-YLPIPERAZIN-1-YL)PYRIMIDINE-2,4,6(2H,3H)-TRIONE; [HYDROXY(3-PHENYLPROPYL)AMINO]METHANOL; [Formylmethyl]Trimethyl-Ammonium, N,N,N-Trimethylammonium Acetaldehyde; [N-(3-DIBENZYLCARBAMOYL-OXIRANECARBONYL)-HYDRAZINO]-ACETIC ACID; [N-(BENZYLOXYCARBONYL)AMINO](4-AMIDINOPHENYL)METHANE-PHOSPHONATE; 5-(5-(2,6-dichloro-4-(4,5-dihydro-2-oxazolyl)phenoxy)pentyl)-3-(hydroxyethyl oxymethyleneoxymethyl) isoxazole; {(2Z)-3-[(6-chloropyridin-3-yl)methyl]-1,3-thiazolidin-2-ylidene}cyanamide; {(2S)-1-[N-(tert-butoxycarbonyl)glycyl]pyrrolidin-2-yl}methyl (3-chlorophenyl)acetate; {(1s)-1-Benzyl-4-[3-Carbamoyl-1-(1-Carbamoyl-2-Phenyl-Ethylcarbamoyl)-(S)-Propylcarbamoyl]-2-Oxo-5-Phenyl-Pentyl}-Carbamic Acid Tert-Butyl Ester; {(2Z)-4-AMINO-2-[(4-METHOXYPHENYL)IMINO]-2,3-DIHYDRO-1,3-THIAZOL-5-YL}(4-METHOXYPHENYL)METHANONE; {[(2,2-Dihydroxy-Ethyl)-(2,3,4,5-Tetrahydroxy-6-Phosphonooxy-Hexyl)-Amino]-Methyl}-Phosphonic Acid; {[(2,6-difluorophenyl)carbonyl]amino}-N-(4-fluorophenyl)-1H-pyrazole-3-carboxamide; 5-[(3R)-3-(5-methoxy-3′,5′-dimethylbiphenyl-3-yl)but-1-yn-1-yl]-6-methylpyrimidine-2,4-diamine; {[4-AMINO-2-(3-CHLOROANILINO)-1,3-THIAZOL-5-YL](4-FLUOROPHENYL)METHANONE; {3-[(3-Hydroxy-2-Methyl-5-Phosphonooxymethyl-Pyridin-4-Ylmethyl)-Amino]-2-Methyl-Propyl}-Phosphonic Acid; {1-[(3-Hydroxy-Methyl-5-Phosphonooxy-Methyl-Pyridin-4-Ylmethyl)-Amino]-Ethyl}-Phosphonic Acid; {1-[2-(1-FORMYL-PROPYL)-3-METHANESULFONYLAMINO-PYRROLIDINE-1-CARBONYL]-2-METHYL-PROPYL}-CARBAMIC ACID TERT-BUTYL ESTER; ([7-(Difluoro-Phosphono-Methyl)-Naphthalen-2-Yl]-Difluoro-Methyl}-Phosphonic Acid; {3-[(4,5,7-TRIFLUORO-1,3-BENZOTHIAZOL-2-YL)METHYL]-1H-INDOL-1-YL}ACETIC ACID; {4-[(2R)-pyrrolidin-2-ylmethoxy]phenyl}(4-thiophen-3-ylphenyl)methanone; {3-[3-(3,4-Dimethoxy-Phenyl)-1-(1-{1-[2-(3,4,5-Trimethoxy-Phenyl)-Butyryl]-Piperidin-2yl)-Vinyloxy)-Propyl]-Phenoxy}-Acetic Acid; {3-[(5-CHLORO-1,3-BENZOTHIAZOL-2-YL)METHYL]-2,4-DIOXO-3,4-DIHYDROPYRIMIDIN-1(2H)-YL}ACETIC ACID; ≡4-[(2s,4e)-2-(1,3-Benzothiazol-2-Yl)-2-(1h-1,2,3-Benzotriazol-1-Yl)-5-Phenylpent-4-Enyl]Phenyl}(Difluoro)Methylphosphonic Acid; {4-[(CARBOXYMETHOXY)CARBONYL]-3,3-DIOXIDO-1-OXONAPHTHO[1,2-D]ISOTHIAZOL-2(1H)-YL}ACETIC ACID; {4-[2,2-BIS(5-METHYL-1,2,4-OXADIAZOL-3-YL)-3-PHENYLPROPYL]PHENYL}SULFAMIC ACID; {4-[2-Acetylamino-2-(3-Carbamoyl-2-Cyclohexylmethoxy-6,7,8,9-Tetrahydro-5h-Benzocyclohepten-5ylcarbamoyl)-Ethyl]-2-Phosphono-Phenyl}-Phosphonic Acid; {4-[2-BENZYL-3-METHOXY-2-(METHOXYCARBONYL)-3-OXOPROPYL]PHENYL}SULFAMIC ACID; 6-({5-fluoro-2-[(3,4,5-trimethoxyphenyl)amino]pyrimidin-4-yl}amino)-2,2-dimethyl-2H-pyrido[3,2-b][1,4]oxazin-3(4H)-one; {4-[3-(6,7-Diethoxy-Quinazolin-4-Ylamino)-Phenyl]-Thiazol-2-Yl}-Methanol; {4-[4-hydroxy-3-(1-methylethyl)benzyl]-3,5-dimethylphenoxy}acetic acid; {5-(5-AMINO-1H-PYRROLO[3,2-B]PYRIDIN-2-YL)-6-HYDROXY-3′-NITRO-BIPHENYL-3-YL]-ACETIC ACID; +/−METHYL 4-(AMINOIMINOMETHYL)-BETA-[3-INH (AMINOIMINO)PHENYL]BENZENE PENTANOATE; 6-(3-AMINOPHENYL)-N-(TERT-BUTYL)-2-(TRIFLUOROMETHYL)QUINAZOLIN-4-AMINE; 1-((1R,2S)-1-{2-[2-(4-CHLOROPHENYL)-1,3-BENZOXAZOL-7-YL]ETHYL}-2-HYDROXYPROPYL)-1H-IMIDAZOLE-4-CARBOXAMIDE; 1-((2-HYDROXYETHOXY)METHYL)-5-(3-(BENZYLOXY)BENZYL)-6-HYDROXYPYRIMIDINE-2,4(1H,3H)-DIONE; 1-((2-HYDROXYETHOXY)METHYL)-5-(3-(BENZYLOXY)BENZYL)PYRIMIDINE-2,4(1H,3H)-DIONE; 1-((2-HYDROXYETHOXY)METHYL)-5-(PHENYLTHIO)PYRIMIDINE-2,4(1H,3H)-DIONE; 6-(2,4-DIAMINO-6-ETHYLPYRIMIDIN-5-YL)-4-(3-METHOXYPROPYL)-2,2-DIMETHYL-2H-1,4-BENZOXAZIN-3(4H)-ONE; 1-({2-[2-(4-CHLOROPHENYL)ETHYL]-1,3-DIOXOLAN-2-YL}METHYL)-1H-IMIDAZOLE; 1-(1,4-dimethyl-1,2,3,4-tetrahydroquinoxalin-6-yl)methanamine; 1-(1′-{[3-(methylsulfanyl)-2-benzothiophen-1-yl]carbonyl}spiro[1-benzofuran-3,4′-piperidin]-5-yl)methanamine; 1-(1-HYDROXY-2,2,6,6-TETRAMETHYLPIPERIDIN-4-YL)PYRROLIDINE-2,5-DIONE; 1-(1-methylethyl)-3-quinolin-6-yl-1H-pyrazolo[3,4-d]pyrimidin-4-amine; 1-(2,3-dihydro-1,4-benzodioxin-6-ylsulfonyl)-4-[(4-methoxyphenyl)sulfonyl]piperazine; 1-(2,5-dideoxy-5-pyrrolidin-1-yl-beta-L-erythro-pentofuranosyl)-5-methylpyrimidine-2,4(1H,3H)-dione; 1-(2,6-Dichlorophenyl)-5-(2,4-Difluorophenyl)-7-Piperazin-1-Yl-3,4-Dihydroquinazolin-2(1h)-One; 8-(6-BROMO-BENZO[1,3]DIOXOL-5-YLSULFANYL)-9-(3-ISOPROPYLAMINO-PROPYL)-ADENINE; 1-(2-{[(6-AMINO-2-METHYLPYRIDIN-3-YL)METHYL]AMINO}ETHYL)-6-CHLORO-3-[(2,2-DIFLUORO-2-PYRIDIN-2-YLETHYL)AMINO]-1,4-DIHYDROPYRAZIN-2-OL; 1-(2-Chlorophenyl)-3,5-Dimethyl-1 h-Pyrazole-4-Carboxylic Acid Ethyl Ester; 1-(2-CYCLOPROPYLETHYL)-3-(1,1-DIOXIDO-2H-1,2,4-BENZOTHIADIAZIN-3-YL)-6-FLUORO-4-HYDROXYQUINOLIN-2(1H)-ONE; 1-(2-Deoxy-2-Fluoro-3-O-Phosphono-Beta-L-Ribofuranosyl)Pyrimidine-2,4(1h,3h)-Dione; 1-(2-DEOXY-5-O-PHOSPHONO-BETA-D-ERYTHRO-PENTOFURANOSYL)-4-METHYL-1H-INDOLE; 1-(2-DEOXY-5-O-PHOSPHONO-BETA-D-ERYTHRO-PENTOFURANOSYL)-5-NITRO-1H-INDOLE; 1-(3-bromophenyl)-7-chloro-6-methoxy-3,4-dihydroisoquinoline; 1-(2-Fluorobenzyl)-3-Butyl-8-(N-Acetyl-4-Aminobenzyl)-Xanthine; 1-(2-HYDROXYETHYLOXYMETHYL)-6-PHENYL THIOTHYMINE; 1-(2-Methoxy-Ethoxy)-2-{2-[2-(2-Methoxy-Ethoxy]-Ethoxy}-Ethane; 1-(3-(2,4-DIMETHYLTHIAZOL-5-YL)-4-OXO-2,4-DIHYDROINDENO[1,2-C]PYRAZOL-5-YL)-3-(4-METHYLPIPERAZIN-1-YL)UREA; 1-(3,5-DICHLOROPHENYL)-5-METHYL-1H-1,2,4-TRIAZOLE-3-CARBOXYLIC ACID; 1-(2-Ethanone)-2-Hydroxy-2-(1-Amino-2-Methyl-2-Ethanol)-4-(2-Dimethyl)Ethane-Imidazoline-5-One; Chromophore (Thr-Leu-Gly); 1-(3-chloro-4-methylphenyl)-3-{2-[({5-[(dimethylamino)methyl]-2-furyl}methyl)thio]ethyl}urea; 1-(3-chlorophenyl)-3-{5-[2-(thieno[3,2-d]pyrimidin-4-ylamino)ethyl]-1,3-thiazol-2-yl}urea; 1-(3-HYDROXYPROPYL)-2-[(3-NITROBENZOYL)AMINO]-1H-BENZIMIDAZOL-5-YL PIVALATE; 1-(3-Mercapto-2-Methyl-Propionyl)-Pyrrolidine-2-Carboxylic Acid; N-(2-M ETHOXYETHYL)-4-({4-[2-METHYL-1-(1-METHYLETHYL)-1H-IMIDAZOL-5-YL]PYRIMIDIN-2-YL}AMINO)BENZENESULFONAMIDE; 1-(4-Aminophenyl)-3,5-Dimethyl-1h-Pyrazole-4-Carboxylic Acid Ethyl Ester; 1-(4-CYANO-PHENYL)-3-[2-(2,6-DICHLORO-PHENYL)-1-IMINO-ETHYL]-THIOUREA; 1-(4-fluorophenyl)-N-[3-fluoro-4-(1H-pyrrolo[2,3-b]pyridin-4-yloxy)phenyl]-2-oxo-1,2-dihydropyridine-3-carboxamide; N-(2-AMINOETHYL)-N˜2˜-{(1S)-1-[4′-(AMINOSULFONYL)BIPHENYL-4-YL]-2,2,2-TRIFLUOROETHYL}-L-LEUCINAMIDE; 1-(4-Methoxyphenyl)-3,5-Dimethyl-1h-Pyrazole-4-Carboxylic Acid Ethyl Ester; 1-(4-Tert-Butylcarbamoyl-Piperazine-1-Carbonyl)-3-(3-Guanidino-Propyl)-4-Oxo-Azetidine-2-Carboxylic Acid; 1-(5-{2-[(1-methyl-1H-pyrazolo[4,3-d]pyrimidin-7-yl)amino]ethyl}-1,3-thiazol-2-yl)-3-[3-(trifluoromethyl)phenyl]urea; 1-(5-BROMO-PYRIDIN-2-YL)-3-[2-(6-FLUORO-2-HYDROXY-3-PROPIONYL-PHENYL)-CYCLOPROPYL]-UREA; 1-(5-Carboxypentyl)-5-[(2,6-Dichlorobenzyl)Oxy]-1H-Indole-2-Carboxylic Acid; N-(3-(8-CYANO-4-(PHENYLAMINO)PYRAZOLO[1,5-A][1,3,5]TRIAZIN-2-YLAMINO)PHENYL)ACETAMIDE; 1-(5-CHLORO-2-METHOXYPHENYL)-3-{6-[2-(DIMETHYLAMINO)-1-METHYLETHOXY]PYRAZIN-2-YL}UREA; 1-(5-Chloroindol-3-Yl)-3-Hydroxy-3-(2h-Tetrazol-5-Yl)-Propenone; N-(3-TERT-BUTYL-1H-PYRAZOL-5-YL)-N′-{4-CHLORO-3-[(PYRIDIN-3-YLOXY)METHYL]PHENYL}UREA; 1-(5-OXO-2,3,5,9B-TETRAHYDRO-1H-PYRROLO[2,1-A]ISOINDOL-9-YL)-3-(5-PYRROLIDIN-2-YL-1H-PYRAZOL-3-YL)-UREA; N′-(5-CHLORO-1,3-BENZODIOXOL-4-YL)-N-(3-MORPHOLIN-4-YLPHENYL)PYRIMIDINE-2,4-DIAMINE; 1-(Isopropylamino)-3-(1-Naphthyloxy)-2-Propanol; 1-(5-Tert-Butyl-2-Methyl-2h-Pyrazol-3-Yl)-3-(4-Chloro-Phenyl)-Urea; N-(5-CHLORO-BENZO[B]THIOPHEN-3-YLMETHYL)-2-[6-CHLORO-OXO-3-(2-PYRIDIN-2-YL-ETHYLAMINO)-2H-PYRAZIN-1-YL]-ACETAMIDE; 1-(6-CYANO-3-PYRIDYLCARBONYL)-5′,8′-DIFLUOROSPIRO[PIPERIDINE-4,2′(1′ H)-QUINAZOLINE]-4′-AMINE; N-(7-CARBAMIMIDOYL-NAPHTHALEN-1-YL)-3-HYDROXY-2-METHYL-BENZAMIDE; 1-(DIMETHYLAMINO)-3-(4-{{4-(2-METHYLIMIDAZO[1,2-A]PYRIDIN-3-YL)PYRIMIDIN-2-YL]AMINO}PHENOXY)PROPAN-2-OL; 1-(HYDROXYMETHYLENEAMINO)-8-HYDROXY-OCTANE; N-(TERT-BUTYL)-3,5-DIMETHYL-N′-[(5-METHYL-2,3-DIHYDRO-1,4-BENZODIOXIN-6-YL)CARBONYL]BENZOHYDRAZIDE; N-[(1S)-2-AMINO-1-(2,4-DICHLOROBENZYL)ETHYL]-5-[2-(METHYLAMINO)PYRIMIDIN-4-YL]THIOPHENE-2-CARBOXAMIDE; 1-(O-Carboxy-Phenylamino)-1-Deoxy-D-Ribulose-5-Phosphate; N-[(1S)-2-{[(1R)-2-(benzyloxy)-1-cyano-1-methylethyl]amino}-1-(cyclohexylmethyl)-2-oxoethyl]morpholine-4-carboxamide; 1(R)-1-ACETAMIDO-2-(3-CARBOXY-2-HYDROXYPHENYL)ETHYL BORONIC ACID; 1(R)-1-Acetamido-2-(3-Carboxyphenyl)Ethyl Boronic Acid; N-[(1S)-1-{1-[(1R,3E)-1-ACETYLPENT-3-EN-1-YL]-1H-1,2,3-TRIAZOL-4-YL}-1,2-DIMETHYLPROPYL]BENZAMIDE; 1,1,1-TRIFLUORO-3-((N-ACETYL)-L-LEUCYLAMIDO)-4-PHENYL-BUTAN-2-ONE(N-ACETYL-L-LEUCYL-L-PHENYLALANYL TRIFLUOROMETHYL KETONE); 1,1,1,3,3,3-HEXAFLUORO-2-{4-[(2,2,2-TRIFLUOROETHYL)AMINO]PHENYL}PROPAN-2-OL; 1,1,1-TRIFLUORO-3-(OCTYLTHIO)ACETONE; 1,1,1-TRIFLUORO-3-ACETAMIDO-4-PHENYL BUTAN-2-ONE(N-ACETYL-L-PHENYLALANYL TRIFLUOROMETHYL KETONE); N-[1-(AMINOMETHYL)CYCLOPROPYL]-3-(MORPHOLIN-4-YLSULFONYL)-N˜2˜-[(1S)-2,2,2-TRIFLUORO-1-(4-FLUOROPHENYL)ETHYL]-L-ALANINAMIDE; 1,2,3-TRIHYDROXY-1,2,3,4-TETRAHYDROBENZO[A]PYRENE; 1,2,5,6-Tetrahydro-4h-Pyrrolo(3,2,1-lj)Quinolin-4-One; 1,2-Di-1-(3,7,11,15-Tetramethyl-Hexadecane)-Sn-Glycero-3-Phosphate; N-[1-(4-CARBAMIMIDOYL-BENZYLCARBAMOYL)-3-METHYLSULFANYL-PROPYL]-3-HYDROXY-2-PROPOXYAMINO-BUTYRAMID; N-[(2R,3S)-1-((2S)-2-{[(CYCLOPENTYLAMINO)CARBONYL]AMINO}-3-METHYLBUTANOYL)-2-(1-FORMYL-1-CYCLOBUTYL)PYRROLIDINYL]CYCLOPROPANECARBOXAMIDE; 1,2-Hydro-1-Oxy-3,4-Hydro-3-(1-Methoxy-2-Oxy-3,4-Dihydroxypentyl)-8,9-Dihydroxy-7-(Sec-Butyl)-Anthracene; 1,3,3-trimethyl-2-[(1E,3E)-3-methylpenta-1,3-dien-1-yl]cyclohexene; 1,3,4,9-Tetrahydro-2-(Hydroxybenzoyl)-9-[(4-Hydroxyphenyl)Methyl]-6-Methoxy-2h-Pyrido[3,4-B]Indole; 1,3,4-TRIHYDROXY-5-(3-PHENOXYPROPYL)-CYCLOHEXANE-1-CARBOXYLIC ACID; 1,3-CYCLOHEXANEDIOL, 4-METHYLENE-5-[(2E)-[(1S,3AS,7AS)-OCTAHYDRO-1-(5-HYDROXY-5-METHYL-1,3-HEXADIYNYL)-7A-METHYL-4H-INDEN-4-YLIDENE]ETHYLIDENE]-, (1R,3S,5Z); 1,3-Di(N-Propyloxy-a-Mannopyranosyl)-Carbomyl 5-Methyazido-Benzene; 1,4-Deoxy-4-((5-Hydroxymethyl-2,3,4-Trihydroxycyclohex-5-Enyl)Amino)Fructose; N-[2-(carbamimidamidooxy)ethyl]-2-{6-cyano-3-[(2,2-difluoro-2-pyridin-2-ylethyl)amino]-2-fluorophenyl}acetamide; 1,5-Bis(N-Benzyloxycarbonyl-L-Leucinyl)Carbohydrazide; 1,6,7,8,9,11A,12,13,14,14A-DECAHYDRO-1,13-DIHYDROXY-6-METHYL-4H-CYCLOPENT[F]OXACYCLOTRIDECIN-4-ONE; N-[3-(2-fluoroethoxy)phenyl]-N′-(1,3,4-trioxo-1,2,3,4-tetrahydroisoquinolin-6-yl)butanediamide; 1-[(2,6-difluorophenyl)sulfonyl]-4-(2,3-dihydro-1,4-benzodioxin-6-ylsulfonyl)piperazine; N-{[2-({[1-(4-CARBOXYBUTANOYL)AMINO]-2-PHENYLETHYL}-HYDROXYPHOSPHINYL)OXY]ACETYL}-2-PHENYLETHYLAMINE; 1-[(2-Amino-6,9-Dihydro-1h-Purin-6-Yl)Oxy]-3-Methyl-2-Butanol; N-[4-(2,4-DIMETHYL-THIAZOL-5-YL)-PYRIMIDIN-2-YL]-N′,N′-DIMETHYL-BENZENE-1,4-DIAMINE; 1-[(2R)-2-aminobutanoyl]-N-(3-chlorobenzyl)-L-prolinamide; N-[5-(ETHYLSULFONYL)-2-METHOXYPHENYL]-5-[3-(2-PYRIDINYL)PHENYL]-1,3-OXAZOL-2-AMINE; 1-[(2S)-2-[(4-CHLOROBENZYL)OXY]-2-(2,4-DICHLOROPHENYL)ETHYL]-1H-IMIDAZOLE; 1-[(2S)-4-(5-BROMO-1H-PYRAZOLO[3,4-B]PYRIDIN-4-YL)MORPHOLIN-2-YL]METHANAMINE; N-[7-(3-AMINOPHENYL)-5-METHOXY-1,3-BENZOXAZOL-2-YL]-2,5-DICHLOROBENZENESULFONAMIDE; 1-[(3S)-5-PHENYL-3-THIOPHEN-2-YL-3H-1,4-BENZODIAZEPIN-2-YL]AZETIDIN-3-OL; 1-[[(1E)-2-(4-CHLOROPHENYL)ETHENYL]SULFONYL]-4-[[1-(4-PYRIDINYL)-4-PIPERIDINYL]METHYL]PIPERAZINE; 1-[1-(3-aminophenyl)-3-tert-butyl-1H-pyrazol-5-yl]-3-naphthalen-1-ylurea; 1-[1-(3-aminophenyl)-3-tert-butyl-1H-pyrazol-5-yl]-3-phenylurea; 1-[1′-(3-phenylacryloyl)spiro[1-benzofuran-3,4′-piperidin]-5-yl]methanamine; 1-[2-(3-ACETYL-2-HYDROXY-6-METHOXY-PHENYL)-CYCLOPROPYL]-3-(5-CYANO-PYRIDIN-2-YL)-THIOUREA; N-[2-Hydroxy-2-(8-Isopropyl-6,9-Dioxo-2-Oxa-7,10-Diaza-Bicyclo[11.2.2]Heptadeca-1(16),13(17),14-Trien-11-Yl)-Ethyl]-N-(3-Methyl-Butyl)-Benzenesulfonamide, Inhibitor 3; 1-[2-(4-ETHOXY-3-FLUOROPYRIDIN-2-YL)ETHYL]-3-(5-METHYLPYRIDIN-2-YL)THIOUREA; 1-[2-(S)-AMINO-3-BIPHENYL-4-YL-PROPIONYL]-PYRROLIDINE-2-(S)-CARBONITRILE; N-[2-methyl-5-(methylcarbamoyl)phenyl]-2-{[(1R)-1-methylpropyl]amino}-1,3-thiazole-5-carboxamide; 1-[2-DEOXYRIBOFURANOSYL]-2,4-DIFLUORO-5-METHYL-BENZENE-5′MONOPHOSPHATE; 1-[2-HYDROXY-3-(4-CYCLOHEXYL-PHENOXY)-PROPYL]-4-(2-PYRIDYL)-PIPERAZINE; 1-[3-({[(4-AMINO-5-FLUORO-2-METHYLQUINOLIN-3-YL)METHYL]THIO}METHYL)PHENYL]-2,2,2-TRIFLUOROETHANE-1,1-DIOL; 1-[3,3-Dimethyl-2-(2-Methylamino-Propionylamino)-Butyryl]-Pyrrolidine-2-Carboxylic Acid(1,2,3,4-Tetrahydro-Naphthalen-1-Yl)-Amide; 1-[4-(4-chlorobenzyl)-1-(7H-pyrrolo[2,3-d]pyrimidin-4-yl)piperidin-4-yl]methanamine; 1-[4-(4-chlorophenyl)-1-(7H-pyrrolo[2,3-d]pyrimidin-4-yl)piperidin-4-yl]methanamine; 1-[4-(AMINOMETHYL)BENZOYL]-5-FLUORO-1′ H-SPIRO[PIPERIDINE-4,2′-QUINAZOLIN]-4′-AMINE; 1-[4-(AMINOSULFONYL)PHENYL]-1,6-DIHYDROPYRAZOLO[3,4-E]INDAZOLE-3-CARBOXAMIDE; 1-[4-(Octahydro-Pyrido[1,2-a]Pyrazin-2-Yl)-Phenyl]-2-Phenyl-1,2,3,4-Tetrahydro-Isoquinolin-6-Ol; 1-[4-(PYRIDIN-4-YLOXY)PHENYL]-3-[3-(TRIFLUOROMETHYL)PHENYL]UREA; 1-[4-Carboxy-2-(3-Pentylamino)Phenyl]-5,5′-Di(Hydroxymethyl)Pyrrolidin-2-One; 1-[5-methyl-2-(trifluoromethyl)furan-3-yl]-3-[(2Z)-5-(2-{[6-(1H-1,2,4-triazol-3-ylamino)pyrimidin-4-yl]amino}ethyl)-1,3-thiazol-2(3H)-ylidene]urea; 1-[6-(2-CHLORO-4-METHYXYPHENOXY)-HEXYL]-IMIDAZOLE; 1-[Glycerolylphosphonyl]-2-[8-(2-Hexyl-Cyclopropyl)-Octanal-1-Yl]-3-[Hexadecanal-1-Yl]-Glycerol; 1-[N[(Phenylmethoxy)Carbonyl]-L-Leucyl-4-[[N/N-[(Phenylmethoxy)Carbonyl]-/NI-Leucyl]Amino]-3-Pyrrolidinone/N; 1-[N-4′-NITROBENZYL-N-4′-CARBOXYBUTYLAMINO]METHYLPHOSPHONIC ACID; 1-[Pyrrol-1-Yl-2,5-Dione-Methoxymethyl]-Pyrrole-2,5-Dione; 1-{(1R,2S)-2-HYDROXY-1-[2-(2-NAPHTHYLOXY)ETHYL]PROPYL}-1H-IMIDAZONE-4-CARBOXAMIDE; D-[3-hydroxy-2-methyl-5-phosphonooxymethyl-pyridin-4-ylmethyl]-N,O-cycloserylamide; 1-{[(1E)-(3-HYDROXY-2-METHYL-5-{[(TRIHYDROXY-LAMBDA\hat{ }5\hat{ }-PHOSPHANYL)OXY]METHYL}PYRIDIN-4-YL)METHYLIDENE]AMINO}UNDECAN-2-ONE; METHYL (2Z)-3-METHOXY-2-{2-[(E)-2-PHENYLVINYL]PHENYL}ACRYLATE; dimethyl (1R,4S)-5,6-bis(4-hydroxyphenyl)-7-oxabicyclo[2.2.1]hepta-2,5-diene-2,3-dicarboxylate; 1-{[N-(1-IMINO-GUANIDINO-METHYL)]SULFANYLMETHYL}-3-TRIFLUOROMETHYL-BENZENE; 1-{2-[(4-CHLOROPHENYL)AMINO]-2-OXOETHYL}-N-(1-ISOPROPYLPIPERIDIN-4-YL)-1H-INDOLE-2-CARBOXAMIDE; 1-{2-[2-(2-Methoxyethoxy)Ethoxy]Ethoxy}-4-(1,1,3,3-Tetramethylbutyl)Benzene; 1-{2-[3-(2-Chloro-4,5-difluoro-benzoyl)-ureido]-4-fluoro-phenyl}-piperidine-4-carboxylic acid; 1-{2-OXO-3-[(1R)-1-(1H-PYRROL-2-YL)ETHYL]-2H-INDOL-5-YL}UREA; 1-{3-[(4-pyridin-2-ylpiperazin-1-yl)sulfonyl]phenyl}-3-(1,3-thiazol-2-yl)urea; 1-{3-oxo-3-[(2S)-2-(pyrrolidin-1-ylcarbonyl)pyrrolidin-1-yl]propyl}-3-phenylquinoxalin-2(1H)-one; 1-{5-[2-(thieno[3,2-d]pyrimidin-4-ylamino)ethyl]-1,3-thiazol-2-yl}-3[3-(trifluoromethyl)phenyl]urea; 1-{7-cyclohexyl-6-[4-(4-methylpiperazin-1-yl)benzyl]-7H-pyrrolo[2,3-d]pyrimidin-2-yl}methanamine; 1044-Dimethylamino-5-Hydroxy-6-Methyl-Tetrahydro-Pyran-2-Yloxy)-8-Ethyl-1,8,11-Trihydroxy-7,8,9,10-Tetrahydro-Naphthacene-5,12-Dione; methyl (1R,2S)-2-(hydroxycarbamoyl)-1-{4-[(2-methylquinolin-4-yl)methoxy]benzyl}cyclopropanecarboxylate; 17Alpha-methyl-3beta,17beta-dihydroxy-5alpha-androstane; 11-[(MERCAPTOCARBONYL)OXY]UNDECANOIC ACID; HYDROXY(OXO)(3-{[(2Z)-4-[3-(1H-1,2,4-TRIAZOL-1-YLMETHYL)PHENYL]PYRIMIDIN-2(5H)-YLIDENE]AMINO}PHENYL)AMMONIUM; 12-(2-hydroxyethyl)-2-(1-methylethoxy)-13,14-dihydronaphtho[2,1-a]pyrrolo[3,4-c]carbazol-5(12H)-one; 17Alpha-methyl-3alpha,17beta-dihydroxy-5alpha-androstane; 10-{4-Dimethylamino-5-[4-Hydroxy-6-Methyl-5-(6-Methyl-5-Oxo-Tetrahydro-Pyran-2-Yloxy)-Tetrahydro-Pyrane-2-Yloxy]-6-Methyl-Tetrahydro-Pyran-2-Yloxy}-8-Ethyl-1,8,11-Trihydroxy-7,8,9,10-Tetrahydro-Naphthacene-5,12-Dione; METHYL N-[(2S,3R)-3-AMINO-2-HYDROXY-3-(4-ISOPROPYLPHENYL)PROPANOYL]-D-ALANYL-D-LEUCINATE; 19-(cyclopropylamino)-4,6,7,15-tetrahydro-5H-16,1-(azenometheno)-10,14-(metheno)pyrazolo[4,3-o][1,3,9]triazacyclohexadecin-8(9H)-one; 17-METHYL-17-ALPHA-DIHYDROEQUILENIN; 18-CHLORO-11,12,13,14-TETRAHYDRO-1H,10H-8,4-(AZENO)-9,15,1,3,6-BENZODIOXATRIAZACYCLOHEPTADECIN-2-ONE; 17-HYDROXY-18A-HOMO-19-NOR-17ALPHA-PREGNA-4,9,11-TRIEN-3-ONE; 1-ACETYL-2-LYSO-SN-GLYCERO-3-PHOSPHOETHANOLAMINE; 1-Acetyl-4-(4-{4-[(2-Ethoxyphenyl)Thio]-3-Nitrophenyl}Pyridin-2-Yl)Piperazine; 1-Allyl-3-Butyl-8-(N-Acetyl-4-Aminobenzyl)-Xanthine; 1-alpha, 25-dihydroxyl-20-epi-22-oxa-24, 26,27-trihomovitamin D3; N-(1-Benzyl-3-{[3-(1,3-Dioxo-1,3-Dihydro-Isoindol-2-Yl)-Propionyl]-[2-(Hexahydro-Benzo[1,3]Dioxol-5-Yl)-Ethyl]-Amino}-2-Hydroxy-Propyl)-4-Benzyloxy-3,5-Dimethoxy-Benzamide; 1-Amino-6-Cyclohex-3-Enylmethyloxypurine; METHYL 3-CHLORO-2-{3-[(2,5-DIHYDROXY-4-METHOXYPHENYL)AMINO]-3-OXOPROPYL}-4,6-DIHYDROXYBENZOATE; 1-Benzyl-3-(4-Methoxy-Benzenesulfonyl)-6-Oxo-Hexahydro-Pyrimidine-4-Carboxylic Acid Hydroxyamide; 1-BENZYL-3-(4-METHOXYPHENYLAMINO)-4-PHENYLPYRROLE-2,5-DIONE; N-({(1R)-1-carboxy-2-[(4-fluorobenzyl)sulfanyl]ethyl}carbamoyl)-L-glutamic acid; 1-Benzyl-5-Methoxy-2-Methyl-1h-Indol-3-Yl)-Acetic Acid; 1-Benzyloxycarbonylamino-2-Phenyl-Ethyl)-{2-[1-Carbamoyl-2-(1h-Indol-3-Yl)-Ethylcarbamoyl]-5-Phenyl-Pentyl}-Phosphinic Acid; N-((1R,2S)-2-(5-CHLORO-1H-INDOLE-2-CARBOXAMIDO)CYCLOHEXYL)-5-METHYL-4,5,6,7-TETRAHYDROTHIAZOLO[5,4-C]PYRIDINE-2-CARBOXAMIDE; N-({6-[(4-CYANO-2-FLUOROBENZYL)OXY]NAPHTHALEN-2-YL}SULFONYL)-D-GLUTAMIC ACID; 1-CHLORO-6-(4-HYDROXYPHENYL)-2-NAPHTHOL; 1-Deoxy-1-Methoxycarbamido-Beta-D-Glucopyranose; 1-CYCLOHEXYL-N-{[1-(4-METHYLPHENYL)-1H-INDOL-3-YL]METHYL}METHANAMINE; 1-cyclopentyl-3-(1H-pyrrolo[2,3-b]pyridin-5-yl)-1H-pyrazolo[3,4-d]pyrimidin-4-amine; 1-DECYL-3-TRIFLUORO ETHYL-SN-GLYCERO-2-PHOSPHOMETHANOL; N-[(13-CYCLOHEXYL-6,7-DIHYDROINDOLO[1,2-D][1,4]BENZOXAZEPIN-10-YL)CARBONYL]-2-METHYL-L-ALANINE; 1-Deoxy-1-Methoxycarbamido-Beta-D-Gluco-2-Heptulopyranosonamide; 1-cyclobutyl-3-(3,4-dimethoxyphenyl)-1H-pyrazolo[3,4-d]pyrimidin-4-amine; 1-Deoxy-6-O-Phosphono-1-[(Phosphonomethyl)Amino]-L-Threo-Hexitol; 1-ETHOXYCARBONYL-D-PHE-PRO-2(4-AMINOBUTYL)HYDRAZINE; 1-ethyl-N-(phenylmethyl)-4-(tetrahydro-2H-pyran-4-ylamino)-1H-pyrazolo[3,4-b]pyridine-5-carboxamide; 1-GUANIDINO-4-(N-NITRO-BENZOYLAMINO-L-LEUCYL-L-PROLYLAMINO)BUTANE; HYDROXY[3-(6-METHYLPYRIDIN-2-YL)PROPYL]FORMAMIDE; 1-Hydroxy-2-S-Glutathionyl-3-Para-Nitrophenoxy-Propane; 1-METHYL ETHYL 1-CHLORO-5-[[(5,6DIHYDRO-2-METHYL-1,4-OXATHIIN-3-YL)CARBONYL]AMINO]MENZOATE; 1-METHYL ETHYL 2-CHLORO-5-[[[(1-METHYLETHOXY)THIOOXO]METHYL]AMINO]-BENZOATE; 1-Methyl-2-Oxy-5,5-Dimethyl Pyrrolidine; 1-methyl-3-naphthalen-2-yl-1H-pyrazolo[3,4-d]pyrimidin-4-amine; N-{2-[(4′-CYANO-1,1′-BIPHENYL-4-YL)OXY]ETHYL}-N′-HYDROXY-N-METHYLUREA; 1-METHYL-3-PHENYL-1H-PYRAZOL-5-YLSULFAMIC ACID; 1-METHYL-3-TRIFLUOROMETHYL-1H-THIENO[2,3-C]PYRAZOLE-5-CARBOXYLIC ACID (2-MERCAPTO-ETHYL)-AMIDE; 1-METHYL-5-(2-PHENOXYMETHYL-PYRROLIDINE-1-SULFONYL)-1H-INDOLE-2,3-DIONE; 1-methyl-8-(phenylamino)-4,5-dihydro-1H-pyrazolo[4,3-h]quinazoline-3-carboxylic acid; 9-CYCLOPENTYL-6-[2-(3-IMIDAZOL-1-YL-PROPOXY)-PHENYLAMINO]-9H-PURINE-2-CARBONITRILE; BENZYL 6-BENZYL-5,7-DIOXO-6,7-DIHYDRO-5H-[1,3]THIAZOLO[3,2-C]PYRIMIDINE-2-CARBOXYLATE; 1-O-[O-Nitrophenyl]-Beta-D-Galactopyranose; 1-Octadecyl-2-Acetamido-2-Deoxy-Sn-Glycerol-3-Phosphoethylmethyl Sulfide; N-{2,2-DIFLUORO-2-[(2R)-PIPERIDIN-2-YL]ETHYL}-2-[2-(1H-1,2,4-TRIAZOL-1-YL)BENZYL][1,3]OXAZOLO[4,5-C]PYRIDIN-4-AMINE; N-({(2S)-1-[(3R)-3-amino-4-(3-chlorophenyl)butanoyl]pyrrolidin-2-yl}methyl)-3-(methylsulfonyl)benzamide; 1-Phenylsulfonamide-3-Trifluoromethyl-5-Parabromophenylpyrazole; 1S,3AS,8AS-TRIMETHYL-1-OXIDO-1,2,3,3A,8,8A-HEXAHYDROPYRROLO[2,3-B]INDOL-5-YL 2-ETHYLPHENYLCARBAMATE; 1-Ter-Butyl-3-P-Tolyl-1h-Pyrazolo[3,4-D]Pyrimidin-4-Ylamine; 1-TERT-BUTYL-3-(2,5-DIMETHYLBENZYL)-1H-PYRAZOLO[3,4-D]PYRIMIDIN-4-AMINE; 1-tert-butyl-3-(3-methylbenzyl)-1H-pyrazolo[3,4-d]pyrimidin-4-amine; 1-Tert-Butyl-3-(4-Chloro-Phenyl)-1h-Pyrazolo[3,4-D]Pyrimidin-4-Ylamine; N-{2-[6-(2,4-DIAMINO-6-ETHYLPYRIMIDIN-5-YL)-2,2-DIMETHYL-3-OXO-2,3-DIHYDRO-4H-1,4-BENZOXAZIN-4-YL]ETHYL}ACETAMIDE; 2-((3,5′-DIMETHYL-4′-HYDROXYPHENYL)AZO)BENZOIC ACID; 2-(2-CHLORO-4-FLUOROPHENOXY)-2-METHYL-N-[(1R,25,35,55,75)-5-(METHYLSULFONYL)-2-ADAMANTYL]PROPANAMIDE; 2-((3′-TERTBUTYL-4′-HYDROXYPHENYL)AZO)BENZOIC ACID; 2-((9H-PURIN-6-YLTHIO)METHYL)-5-CHLORO-3-(2-METHOXYPHENYL)QUINAZOLIN-4(3H)-ONE; 2-({[3-(3,4-dihydroisoquinolin-2(1H)-ylsulfonyl)phenyl]carbonyl}amino)benzoic acid; 2-({[2,3,5,6-TETRAFLUORO-3-(TRIFLUOROMETHOXY)BIPHENYL-4-YL]AMINO}CARBONYL)CYCLOPENTA-1,3-DIENE-1-CARBOXYLIC ACID; 2-({[3,5-DIFLUORO-3-(TRIFLUOROMETHOXY)BIPHENYL-4-YL]AMINO}CARBONYL)CYCLOPENT-1-ENE-1-CARBOXYLIC ACID; 2-({[4-(TRIFLUOROMETHOXY)PHENYL]SULFONYL}AMINO)ETHYL DIHYDROGEN PHOSPHATE; 2-({[4-bromo-3-(diethylsulfamoyl)phenyl]carbonyl}amino)benzoic acid; 2-({2-[(3-HYDROXYPHENYL)AMINO]PYRIMIDIN-4-YL}AMINO)BENZAMIDE; N-{3-[4-(3-Amino-Propyl)-Piperazin-1-Yl]-Propyl}-3-Nitro-5-(Galactopyranosyl)-Alpha-Benzamide; 2-({4-[(5-CHLORO-1H-INDOL-2-YL)SULFONYL]PIPERAZIN-1-YL}CARBONYL)THIENO[3,2-B]PYRIDINE 4-OXIDE; 2-({4-[4-(pyridin-4-ylmethyl)-1H-pyrazol-3-yl]phenoxy}methyl)quinoline; 2-({5-CHLORO-2-[(2-METHOXY-4-MORPHOLIN-4-YLPHENYL)AMINO]PYRIMIDIN-4-YL}AMINO)-N-METHYLBENZAMIDE; 2-({8-[(3R)-3-AMINOPIPERIDIN-1-YL]-1,3-DIMETHYL-2,6-DIOXO-1,2,3,6-TETRAHYDRO-7H-PURIN-7-YL}METHYL)BENZONITRILE; 2-(1,1-DIMETHYLETHYL)9-FLUORO-3,6-DIHYDRO-7H-BENZ[H]-IMIDAZ[4,5-F]ISOQUINOLIN-7-ONE; 2-(1,3-benzodioxol-5-yl)-5-[(3-fluoro-4-methoxybenzyl)sulfanyl]-1,3,4-oxadiazole; 2-(11-{2-[Benzenesulfonyl-(3-Methyl-Butyl)-Amino]-1-Hydroxy-Ethyl}-6,9-Dioxo-2-Oxa-7,10-Diaza-Bicyclo[11.2.2]Heptadeca-1(16),13(17),14-Trien-8-Yl)-Acetamide, Inhibitor 2; 2-(1H-imidazol-1-yl)-9-methoxy-8-(2-methoxyethoxy)benzo[c][2,7]naphthyridin-4-amine; 2-(2,4-DICHLOROPHENOXY)-5-(PYRIDIN-2-YLMETHYL)PHENOL; N˜2˜-[(BENZYLOXY)CARBONYL]-N-[(1S,2S)-2-HYDROXY-1-(4-HYDROXYBENZYL)PROPYL]-L-LEUCINAMIDE; N˜3˜cyclopropyl-N˜4˜′-(cyclopropylmethyl)-6-methylbiphenyl-3,4′-dicarboxamide; N˜2˜,N˜2˜-DIMETHYL-N-1--(6-OXO-5,6-DIHYDROPHENANTHRIDIN-2-YL)GLYCINAMIDE; 2-(2-{2-[2-(2-{2-[2-(2-Ethoxy-Ethoxy)-Ethoxy]-Ethoxy}-Ethoxy)-Ethoxy]-Ethoxy}-Ethoxy)-Ethanol, Polyethyleneglycol Peg400; 2-(2-{2-[2-(2-Methoxy-Ethoxy)-Ethoxy]-Ethoxy}-Ethoxy)-Ethanol; 2-((3-METHYL-4′-HYDROXYPHENYL)AZO)BENZOIC ACID; 2-(2-chloropyridin-4-yl)-4-methyl-1H-isoindole-1,3(2H)-dione; 2-(2f-Benzothiazolyl)-5-Styryl-3-(4f-Phthalhydrazidyl)Tetrazolium Chloride; 2-(2-Hydroxy-1,1-Dihydroxymethyl-Ethylamino)-Ethanesulfonic Acid; 2-(2-Hydroxy-5-Methoxy-Phenyl)-1h-Benzoimidazole-5-Carboxamidine; 2-(2-HYDROXY-BIPHENYL)-1H-BENZOIMIDAZOLE-5-CARBOXAMIDINE; 2-(2-HYDROXYETHYLAMINO)-6-(3-CHLOROANILINO)-9-ISOPROPYLPURINE; 2-(2-Hydroxy-Phenyl)-1h-Benzoimidazole-5-Carboxamidine; 2-(2-Hydroxy-Phenyl)-1h-Indole-5-Carboxamidine; 2-(2-Hydroxy-Phenyl)-3h-Benzoimidazole-5-Carboxamidine; 2-(2-METHYLPHENYL)-1H-INDOLE-5-CARBOXIMIDAMIDE; 2-(2-METHYLPHENYL)-1H-INDOLE-6-CARBOXIMIDAMIDE; 2-(2-Oxo-1,2-Dihydro-Pyridin-3-Yl)-1h-Benzoimidazole-5-Carboxamidine; N-3-OXO-DODECANOYL-L-HOMOSERINE LACTONE; 2-(2-QUINOLIN-3-YLPYRIDIN-4-YL)-1,5,6,7-TETRAHYDRO-4H-PYRROLO[3,2-C]PYRIDIN-4-ONE; 2-(3-((4,5,7-trifluorobenzo[d]thiazol-2-yl)methyl)-1H-pyrrolo[2,3-b]pyridin-1-yl)acetic acid; 2-(3,4-DIHYDROXYPHENYL)-8-(1,1-DIOXIDOISOTHIAZOLIDIN-2-YL)-3-HYDROXY-6-METHYL-4H-CHROMEN-4-ONE; 2-(3,4-Dihydro-3-Oxo-2h-Benzo[B][1,4]Thiazin-2-Yl)-N-Hydroxyacetamide; 2-(3-AMINO-2,5,6-TRIMETHOXYPHENYL)ETHYL 5-CHLORO-2,4-DIHYDROXYBENZOATE; 2-(3-BENZOYLPHENOXY)ETHYL(HYDROXY)FORMAMIDE; 2-(3-BROMOPHENYL)-6-[(2-HYDROXYETHYL)AMINO]-1H-BENZO[DE]ISOQUINOLINE-1,3(2H)-DIONE; 2-(3-CARBOXYPROPIONYL)-6-HYDROXY-CYCLOHEXA-2,4-DIENE CARBOXYLIC ACID; 2-(3-CHLORO-6-{[2,2-DIFLUORO-2-(1-OXIDOPYRIDIN-2-YL)ETHYL]AMINO}-1-OXIDOPYRIDIN-2-YL)-N-[1-(3-CHLOROPHENYL)ETHYL]ACETAMIDE; 2-(3-FLUORO-4-HYDROXYPHENYL)-7-VINYL-1,3-BENZOXAZOL-5-OL; 2-(3-GUANIDINOPHENYL)-3-MERCAPTOPROPANOIC ACID; N-ALLYL-5-AMIDINOAMINOOXY-PROPYLOXY-3-CHLORO-N-CYCLOPENTYLBENZAMIDE; 2-(3-METHYLPHENYL)-1H-INDOLE-5-CARBOXIMIDAMIDE; 2-(3-OXO-PROPYLSULFANYLCARBONYL)-ETHANETHIOLATE; 2-(4-(2-((3-(5-(PYRIDIN-2-YLTHIO)THIAZOL-2-YL)UREIDO)METHYL)-1H-IMIDAZOL-4-YL)PHENOXY)ACETIC ACID; 2-(4-(2-HYDROXY-3-(ISOPROPYLAMINO)PROPOXY)PHENYL)ETHANAMIDE; 2-(4-(AMINOMETHYL)PIPERIDIN-1-YL)-N-(3_CYCLOHEXYL-4-OXO-2,4-DIHYDROINDENO[1,2-C]PYRAZOL-5-YL)ACETAMIDE; 2-(4-{(3S,5S)-5-[(3,3-difluoropyrrolidin-1-yl)carbonyl]pyrrolidin-3-yl}piperazin-1-yl)pyrimidine; 2-(4-CARCOXY-5-ISOPROPYLTHIAZOLYL)BENZOPIPERIDINE; N-BENZYLOXYCARBONYL-ALA-PRO-3-AMINO-4-PHENYL-BUTAN-2-OL; 2-(4-Chlorophenyl)-5-Quinoxalinecarboxamide; 2-(4-CHLORO-PHENYLAMINO)-NICOTINIC ACID; 2′-(4-Dimethylaminophenyl)-5-(4-Methyl-1-Piperazinyl)-2,5′-Bi-Benzimidazole; 2-(4-DIMETHYLAMINOPHENYL)DIAZENYLBENZOIC ACID; 2-(4-ETHYLPIPERAZIN-1-YL)-4-(PHENYLAMINO)PYRAZOLO[1,5-A][1,3,5]TRIAZINE-8-CARBONITRILE; 2-(4-fluorophenyl)-N-{[3-fluoro-4-(1H-pyrrolo[2,3-b]pyridin-4-yloxy)phenyl]carbamoyl}acetamide; N-METHYL O-NITROPHENYL AMINOETHYLDIPHOSPHATE BERYLLIUM TRIFLUORIDE; 2-(4-HYDROXY-5-PHENYL-1H-PYRAZOL-3-YL)-1H-BENZOIMIDAZOLE-5-CARBOXAMIDINE; 2-(4-hydroxybiphenyl-3-yl)-4-methyl-1H-isoindole-1,3(2H)-dione; Phosphoric Acid Mono-[2-Amino-3-(3h-Imidazol-4-Yl)-Propyl]Ester; 2-(4-Morpholinyl)-8-Phenyl-4h-1-Benzopyran-4-One; Phosphoric Acid Mono-[3-Amino-5-(5-Methyl-2,4-Dioxo-3,4-Dihydro-2h-Pyrimidin-1-Yl)-Tetrahydro-Furan-2-Ylmethyl] Ester; 2-(6-{[(3-chloro-2-methylphenyl)sulfonyl]amino}pyridin-2-yl)-N,N-diethylacetamide; 2-(5-HYDROXY-NAPHTHALEN-1-YL)-1,3-BENZOOXAZOL-6-OL; 2-(5-CHLORO-2-THIENYL)-N-{(3S)-1-[(1S)-1-METHYL-2-MORPHOLIN-4-YL-2-OXOETHYL]-2-OXOPYRROLIDIN-3-YL}ETHENESULFONAMIDE; 2-(6-CHLORO-3-{[2,2-DIFLUORO-2-(1-OXIDO-2-PYRIDINYL)ETHYL]AMINO}-2-OXO-1(2H)-PYRAZINYL)-N-[(2-FLUOROPHENYL)METHYL]ACETAMIDE; 2-(6-CHLORO-3-{[2,2-DIFLUORO-2-(2-PYRIDINYL)ETHYL]AMINO}-2-OXO-1(2H)-PYRAZINYL)-N-[(2-FLUORO-3-METHYL-6-PYRIDINYL)METHYL]ACETAMIDE; 2-(6-CHLORO-3-{[2,2-DIFLUORO-2-(2-PYRIDINYL)ETHYL]AMINO}-2-OXO-1(2H)-PYRAZINYL)-N-[(2-FLUORO-6-PYRIDINYL)METHYL]ACETAMIDE; Phosphoric Acid Mono-[3-Fluoro-5-(5-Methyl-2,4-Dioxo-3,4-Dihydro-2h-Pyrimidin-1-Yl)-Tetrahyro-Furan-2-Ylmethyl] Ester; 2′,3-Dehydro-2′,3′-Deoxy-Thymidine 5-Triphosphate; 2-(Acetylamino)-2-Deoxy-4-O-Beta-D-Galactopyranosyl-Alpha-D-Glucopyranose; 2-(Acetylamino)-2-Deoxy-4-O-Sulfo-Alpha-D-Galactopyranose; 2-(Acetylamino)-2-Deoxy-6-O-Methyl-Alpha-D-Allopyranose; 2-(ACETYL-HYDROXY-AMINO)-4-METHYL-PENTANOIC ACID METHYL ESTER; 2-(Beta-D-Glucopyranosyl)-5-Methyl-1,2,3-Benzimidazole; 2-(Beta-D-Glucopyranosyl)-5-Methyl-1,3,4-Benzothiazole; TRANS-4-(GUANIDINOMETHYL)-CYCLOHEXANE-L-YL-D-3-CYCLOHEXYLALANYL-L-AZETIDINE-2-YL-D-TYROSINYL-L-HOMOARGININAMIDE; S-(2-{[N-(2-HYDROXY-4-{[HYDROXY(OXIDO)PHOSPHINO]OXY}-3,3-DIMETHYLBUTANOYL)-BETA-ALANYL]AMINO}ETHYL) HEPTANETHIOATE; SUCCINIC ACID MONO-(13-METHYL-3-OXO-2,3,6,7,8,9,10,11,12,13,14,15,16,17-TETRADECAHYDRO-1H-CYCLOPENTA[A]PHENANTHREN-17-YL) ESTER; 2-(Carboxymethoxy)-5-[(2s)-2-({(2s)-2-[(3-Carboxypropanoyl)Amino]-3-Phenylpropanoyl}Amino)-3-Oxo-3-(Pentylamino)Propyl]Benzoic Acid; 2-(CARBOXYMETHYL)-1-OXO-1,2-DIHYDRONAPHTHO[1,2-D]ISOTHIAZOLE-4-CARBOXYLIC ACID 3,3-DIOXIDE; 2-(cycloheptylmethyl)-1,1-dioxido-1-benzothiophen-6-yl sulfamate; 2-(CYCLOHEXYLMETHYLAMINO)-4-(PHENYLAMINO)PYRAZOLO[1,5-A][1,3,5]TRIAZINE-8-CARBONITRILE; 2-(ETHOXYMETHYL)-4-(4-FLUOROPHENYL)-3-[2-(2-HYDROXYPHENOXY)PYRIMIDIN-4-YL]ISOXAZOL-5(2H)-ONE; Pinacol[[2-Amino-Alpha-(1-Carboxy-1-Methylethoxyimino)-4-Thiazoleacetyl]Amino]Methaneboronate; N-ethyl-4-{[5-(methoxycarbamoyl)-2-methylphenyl]amino}-5-methylpyrrolo[2,1-f][1,2,4]triazine-6-carboxamide; 2-(methylsulfanyl)-5-(thiophen-2-ylmethyl)-1H-imidazol-4-ol; 2-(Oxalyl-Amino)-4,5,6,7-Tetrahydro-Thieno[2,3-C]Pyridine-3-Carboxylic Acid; 2-(Oxalyl-Amino)-4,7-Dihydro-5h-Thieno[2,3-C]Pyran-3-Carboxylic Acid; 2-(Oxalyl-Amino)-4,7-Dihydro-5h-Thieno[2,3-C]Thiopyran-3-Carboxylic Acid; 2-(TOLUENE-4-SULFONYL)-2H-BENZO[D][1,2,3]DIAZABORININ-1-OL; 2,2,2-TRIFLUORO-1-{5-[(3-PHENYL-5,6-DIHYDROIMIDAZO[1,2-A]PYRAZIN-7(8H)-YL)CARBONYL]THIOPHEN-2-YL}ETHANE-1,1-DIOL; 2,2,5,5-TETRAMETHYL-3-(SULFANYLMETHYL)-2,5-DIHYDRO-1H-PYRROL-1-OL; TERT-BUTYL {2-[(1,3-THIAZOL-2-YLAMINO)CARBONYL]PYRIDIN-3-YL}CARBAMATE; 2,2-Dichloro-1-Methanesulfinyl-3-Methyl-Cyclopropanecarboxylic Acid [1-(4-Bromo-Phenyl)-Ethyl]-Amide; 2,2-Dimethylthiazolidine-4-Carboxylic Acid; (Dmt)Thiazolidine; 2,3,17BETA-TRIHYDROXY-1,3,5(10)-ESTRATRIENE; 2,4-DIAMINO-1,5-DIPHENYL-3-HYDROXYPENTANE; 2,3-Bis-Benzo[1,3]Dioxol-5-Ylmethyl-Succinic Acid; TERT-BUTYL 2-CYANO-2-METHYLHYDRAZINECARBOXYLATE; 2-(Beta-D-Glucopyranosyl)-5-Methyl-1,3,4-Oxadiazole; 2,3-Dicarboxy-4-(2-Chloro-Phenyl)-1-Ethyl-5-Isopropoxycarbonyl-6-Methyl-Pyridinium; 2,3-DIMETHOXY-12H-[1,3]DIOXOLO[5,6]INDENO[1,2-C]ISOQUINOLIN-6-IUM; 2,3-Di-O-Phytanly-3-Sn-Glycero-1-Phosphoryl-3′-Sn-Glycerol-1′-Phosphate; 2,3-DIPHENYL-N-(2-PIPERAZIN-1-YLETHYL)FURO[2,3-13]PYRIDIN-4-AMINE; 2,4-Deoxy-4-Guanidine-5-N-Acetyl-Neuraminic Acid; 2,3,6A,7,8,9-HEXAHYDRO-11H-[1,4]DIOXINO[2,3-G]PYRROLO[2,1-B][1,4]BENZOXAZIN-11-ONE; 2,4-Diamine-5-(3,4,5-Trimethoxy-Benzyl)-Pyrimidin-1-lum; 2,4-Diamino-5-Methyl-6-[(3,4,5-Trimethoxy-N-Methylanilino)Methyl]Pyrido[2,3-D]Pyrimidine; 2,4-Diamino-6-[N-(2′,5′-Dimethoxybenzyl)-N-Methylamino]Quinazoline; 2,4-Diamino-6-[N-(3′,4′,5′-Trimethoxybenzyl)-N-Methylamino]Pyrido[2,3-D]Pyrimidine; 2,4-Diamino-6-[N-(3′,5′-Dimethoxybenzyl)-N-Methylamino]Pyrido[2,3-D]Pyrimidine; S-[2-({N-[(2S)-2-hydroxy-3,3-dimethyl-4-(phosphonooxy)butanoyl]-beta-alanyl}amino)ethyl] hexanethioate; 2′,4′-DICHLORO-4-HYDROXY-1,1′-BIPHENYL-3-CARBOXYLIC ACID; 2,4-Difluorobenzyl Alcohol 2,4-Difluoro-1-(Hydroxymethyl)Benzene; 2,4-Dihydroxy-7-(Methyloxy)-2h-1,4-Benzoxazin-3(4h)-One; 2,4-Dinitro,5-[Bis(2-Bromoethyl)Amino]-N-(2′,3′-Dioxopropyl)Benzamide; N′-[(1E)-(3,5-DIBROMO-2,4-DIHYDROXYPHENYL)METHYLENE]NICOTINOHYDRAZIDE; 2-[(3-Hydroxy-2-Methyl-5-Phosphonooxymethyl-Pyridin-4-Ylmethyl)-Imino]-5-Phosphono-Pent-3-Enoic Acid; 4-(6-CHLORO-2,4-DIOXO-1,2,3,4-TETRAHYDROPYRIMIDIN-5-YL) BUTYL PHOSPHATE; 2,5-bis-o-{3-[amino(imino)methyl]phenyl}-1,4:3,6-dianhydro-d-glucitol; 2,5-DI-(TERT-BUTYL)-1,4,BENZOHYDROQUINONE; 2,5-Diaziridin-1-Yl-3-(Hydroxymethyl)-6-Methylcyclohexa-2,5-Diene-1,4-Dione; 2,5-dichloro-N-(3,5-dibromo-4-hydroxyphenyl)benzamide; 2,5-DICHLORO-N-(5-CHLORO-1,3-BENZOXAZOL-2-YL)BENZENESULFONAMIDE; 2,5-DICHLORO-N-[5-METHOXY-7-(6-METHOXYPYRIDIN-3-YL)-1,3-BENZOXAZOL-2-YL]BENZENESULFONAMIDE; 2′,5′-DIDEOXY-ADENOSINE 3′-MONOPHOSPHATE; 2,5-Dimethoxy-4-(n)-propylthiophenethylamine; 2,5-DIPHENYLFURAN-3,4-DICARBOXYLIC ACID; 2,5-O,O-BIS-(4′,4″-AMIDINOPHENYL)-1,4:3,6-DIANHYDRO-D-SORBITOL; 2,6,8-Trimethyl-3-Amino-9-Benzyl-9-Methoxynonanoic Acid; N-[(6-BUTOXYNAPHTHALEN-2-YL)SULFONYL]-L-GLUTAMIC ACID; 2,6-Anhydro-3-Deoxy-D-Erythro-Hex-2-Enonic Acid; 2,6-Diamino-(S)-9-[2-(Phosphonomethoxy)Propyl]Purine; 2,6-diamine-1,7-dihydro-8H-imidazo[4,5-g]quinazolin-8-one; 2,6-Diamino-8-(2-Dimethylaminoethylsulfanylmethyl)-3h-Quinazolin-4-One; 2,6-Diamino-8-(1h-Imidazol-2-Ylsulfanylmethyl)-3h-Quinazoline-4-One; 2,6-Diamino-8-Propylsulfanylmethyl-3h-Quinazoline-4-One; 2,6-dibromo-4-[(E)-2-phenylethenyl]phenol; 2′,6′-DIFLUOROBIPHENYL-4-CARBOXYLIC ACID; 4-{4-[(5-hydroxy-2-methylphenyl)amino]quinolin-7-yl}-1,3-thiazole-2-carbaldehyde; 4-AMINO-5-(2-METHYLPHENYL)-2,4-DIHYDRO-3H-1,2,4-TRIAZOLE-3-THIONE; 2,6-DIMETHYL-1-(3-[3-METHYL-5-ISOXAZOLYL]-PROPANYL)-4-[2N-METHYL-2H-TETRAZOL-5-YL]-PHENOL; 2,6-DIMETHYL-1-(3-[3-METHYL-5-ISOXAZOLYL]-PROPANYL)-4-[4-METHYL-2H-TETRAZOL-2-YL]-PHENOL; 2,6-dimethyl-4-[(E)-2-phenylethenyl]phenol; 2-[(1R)-2-carboxy-1-(naphthalen-1-ylmethyl)ethyl]-1,3-dioxo-2,3-dihydro-1H-isoindole-5-carboxylic acid; 2-[({4-[2-(trifluoromethyl)phenyl]piperidin-1-yl}carbonyl)amino]benzoic acid; 2-[(1R)-1-CARBOXY-2-(4-HYDROXYPHENYL)ETHYL]-1,3-DIOXOISOINDOLINE-5-CARBOXYLIC ACID; 2-[(1R)-1-carboxy-2-naphthalen-1-ylethyl]-1,3-dioxo-2,3-dihydro-1H-isoindole-5-carboxylic acid; 2-[({2-[(1Z)-3-(DIMETHYLAMINO)PROP-1-ENYL]-4-FLUOROPHENYL}SULFONYL)AMINO]-5,6,7,8-TETRAHYDRONAPHTHALENE-1-CARBOXYLIC ACID; 4-[3-Carboxymethyl-3-(4-Phosphonooxy-Benzyl)-Ureido]-4-[(3-Cyclohexyl-Propyl)-Methyl-Carbamoyl]Butyric Acid; 2-[(2′,3,4′-TRIFLUOROBIPHENYL-2-YL)OXY]ETHANOL; 2-[(2,4-DICHLORO-5-METHYLPHENYL)SULFONYL]-1,3-DINITRO-5-(TRIFLUOROMETHYL)BENZENE; 2-[(2,4-DICHLOROBENZOYL)AMINO]-5-(PYRIMIDIN-2-YLOXY)BENZOIC ACID; 4-(2-(1H-IMIDAZOL-4-YL)ETHYLAMINO)-2-(PHENYLAMINO)PYRAZOLO[1,5-A][1,3,5]TRIAZINE-8-CARBONITRILE; 2-[(2e,6e,10e,14e,18e,22e,26e)-3,7,11,15,19,23,27,31-Octamethyldotriaconta-2,6,10,14,18,22,26,30-Octaenyl]Phenol; 2-[(2-methoxy-5-methylphenoxy)methyl]pyridine; 2-[(2-methoxyethyl)amino]-4-(4-oxo-1,2,3,4-tetrahydro-9H-carbazol-9-yl)benzamide; 4-{2,6,8-Trioxo-9-[(2r,3s,4r)-2,3,4,5-Tetrahydroxypentyl]-1,2,3,6,8,9-Hexahydro-7h-Purin-7-Yl}Butyl Dihydrogen Phosphate; 2-[(2-Oxo-2-Piperidin-1-Ylethyl)Thio]-6-(Trifluoromethyl)Pyrimidin-4(1h)-One; 2-[(3,5-Dichloro-4-trioxidanylphenyl)amino]benzoic acid; 2,4-DINITROPHENYL 2-DEOXY-2-FLUORO-BETA-D-MANNOPYRANOSIDE; 2-[(4-ETHYNYL-2-FLUOROPHENYL)AMINO]-3,4-DIFLUORO-N-(2-HYDROXYETHOXY)BENZAMIDE; 4-({4-[(4-AMINOBUT-2-YNYL)OXY]PHENYL}SULFONYL)-N-HYDROXY-2,2-DIMETHYLTHIOMORPHOLINE-3-CARBOXAMIDE; 2-[(5,6-DIPHENYLFURO[2,3-D]PYRIMIDIN-4-YL)AMINO]ETHANOL; 2-[(5-hex-1-yn-1-ylfuran-2-yl)carbonyl]-N-methylhydrazinecarbothioamide; 2-[(7-HYDROXY-NAPHTHALEN-1-YL)-OXALYL-AMINO]-BENZOIC ACID; 2-[(CYCLOPROPYLCARBONYL)AMINO]-4,5,6,7-TETRAHYDRO-1-BENZOTHIOPHENE-3-CARBOXAMIDE; 4-(2-aminoethoxy)-N-(3-chloro-5-piperidin-1-ylphenyl)-3,5-dimethylbenzamide; 2-[(PHENYLSULFONYL)AMINO]-5,6,7,8-TETRAHYDRONAPHTHALENE-1-CARBOXYLIC ACID; 2-[1-(4-CHLOROBENZOYL)-5-METHOXY-2-METHYL-1H-INDOL-3-YL]-N-[(1R)-1-(HYDROXYMETHYL)PROPYL]ACETAMIDE; 5,6,7,8,9,10-HEXAHYDRO-4-HYDROXY-3-(1-PHENYLPROPYL)CYCLOOCTA[B]PYRAN-2-ONE; 2-[1-(4-CHLORO-PHENYL)-ETHYL]-4,6-DINITRO-PHENOL; 2-[1-METHYLHEXYL]-4,6-DINITROPHENOL; 2-[2-(1,3-Dioxo-1,3-Dihydro-2h-Isoindol-2-Yl)Ethyl]-4-(4′-Ethoxy-1,1′-Biphenyl-4-Yl)-4-Oxobutanoic Acid; 2-[3-({ethyl[1-(2-Naphthoyl)Piperidin-4-Yl]Amino}Carbonyl)-2-Naphthyl]-1-(1-Naphthyl)-2-Oxoethylphosphonic Acid; 2-[2-(2-Cyclohexyl-2-Guanidino-Acetylamino)-Acetylamino]-N-(3-Mercapto-Propyl)-Propionamide; 2-[2-(2-FLUOROPHENYL)PYRIDIN-4-YL]-1,5,6,7-TETRAHYDRO-4H-PYRROLO[3,2-C]PYRIDIN-4-ONE; 5,6-DIPHENYL-N-(2-PIPERAZIN-1-YLETHYL)FURO[2,3-D]PYRIMIDIN-4-AMINE; 5-AMINO-6-CYCLOHEXYL-4-HYDROXY-2-ISOPROPYL-HEXANOIC ACID; 2-[2-ETHANESULFONYLAMINO-3-(1H-INDOL-3-YL)-PROPIONYLAMINO]-PENTANEDIOIC ACID 5-AMIDE 1-(4-CARBAMIMIDOYL-BENZYLAMIDE); 2-[2-ETHANESULFONYLAMINO-3-(5-PROPOXY-1H-INDOL-3-YL)-PROPIONYLAMINO]-PENTANEDIOIC ACID 5-AMIDE 1-(4-CARBAMIMIDOYL-BENZYLAMIDE); 2-[2-(1H-tetrazol-5-yl)ethyl]-1H-isoindole-1,3(2H)dione; 2-[3-(2-Hydroxy-1,1-Dihydroxymethyl-Ethylamino)-Propylamino]-2-Hydroxymethyl-Propane-1,3-Diol; 2-[3-(5-MERCAPTO-[1,3,4]THIADIAZOL-2YL)-UREIDO]-N-METHYL-3-PENTAFLUOROPHENYL-PROPIONAMIDE; 2-[3-(5-MERCAPTO-[1,3,4]THIADIAZOL-2-YL)-UREIDO]-N-METHYL-3-PHENYL-PROPIONAMIDE; 2-[3,4-Dihydroxy-2-Hydroxymethyl-5-(2-Hydroxy-Nonyl)-Tetrahydro-Furan-2-Yloxy]-6-Hydroxymethyl-Tetra Hydro-Pyran-3,4,5-Triol; 2-[4-({[(3,5-DICHLOROPHENYL)AMINO]CARBONYL}AMINO)PHENOXY]-2-METHYLPROPANOIC ACID; 2-[4(2,4-Dichlorophenoxy)Phenoxy]Propanoic Acid; 2-[4-(2H-1,4-BENZOTHIAZINE-3-YL)-PIPERAZINE-1-LY]-1,3-THIAZOLE-4-CARBOXYLIC ACID ETHYLESTER; 2-[4-(3-METHYL-1H-PYRAZOL-4-YL)PHENYL]ETHANAMINE; 2-[4-(4-Chlorophenyl)Cyclohexylidene]-3,4-Dihydroxy-1(2h)-Naphthalenone; 2-[4-(4-Hydroxy-3-Isopropyl-Phenoxy)-3,5-Dimethyl-Phenyl]-2h-[1,2,4]Triazine-3,5-Dione; 2-[4-(DIMETHYLAMINO)PHENYL]-6-HYDROXY-3-METHYL-1,3-BENZOTHIAZOL-3-IUM; 2-[4-(Hydroxy-Methoxy-Methyl)-Benzyl]-7-(4-Hydroxymethyl-Benzyl)-1,1-Dioxo-3,6-Bis-Phenoxymethyl-1lambda6-[1,2,7]Thiadiazepane-4,5-Diol; 6-CYCLOHEXYLMETHYLOXY-2-(4′-HYDROXYANILINO)PURINE; 2[4-BROMO-2-FLUOROPHENYL)METHYL]-6-FLUOROSPIRO[ISOQUINOLINE-4-(1H),3-PYRROLIDINE]-1,2′,3,5′(2H)-TETRONE; 2-[4-chloro-2-(phenylcarbonyl)phenoxy]-N-phenylacetamide; 2-[5,6-BIS-(4-METHOXY-PHENYL)-FURO[2,3-D]PYRIMIDIN-4-YLAMINO]-ETHANOL; 2-[5-Hydroxy-3-Methyl-1-(2-Methyl-4-Sulfo-Phenyl)-1h-Pyrazol-4-Ylazo]-4-Sulfo-Benzoic Acid; 2-[5-Methanesulfonylamino-2-(4-Aminophenyl)-6-Oxo-1,6-Dihydro-1-Pyrimidinyl]-N-(3,3,3-Trifluoro-1-Isopropyl-2-Oxopropyl)Acetamide; 6-(CYCLOHEXYLMETHOXY)-8-ISOPROPYL-9H-PURIN-2-AMINE; 2-[METHYL-(5-GERANYL-4-METHYL-PENT-3-ENYL)-AMINO]-ETHYL-DIPHOSPHATE; 6-amino-2-[(1-naphthylmethyl)amino]-3,7-dihydro-8H-imidazo[4,5-g]quinazolin-8-one; 2-[Trans-(4-Aminocyclohexyl)Amino]-6-(Benzyl-Amino)-9-Cyclopentylpurine; 2-{(9as)-9a-[(1s)-1-Hydroxyethyl]-2,7-Dimethyl-9a,10-Dihydro-5h-Pyrimido[4,5-D][1,3]Thiazolo[3,2-a]Pyrimidin-8-Yl}Ethyl Trihydrogen Diphosphate; 2-{[(1R,2S)-2-aminocyclohexyl]amino}-4-[(3-methylphenyl)amino]pyrimidine-5-carboxamide; 2-{[(4-CHLOROPHENOXY)ACETYL]AMINO}BENZOIC ACID; 6-(DIFLUORO-PHOSPHONO-METHYL)-NAPHTHALENE-2-CARBOXYLIC ACID; 2-{[4-(1-methyl-4-pyridin-4-yl-1H-pyrazol-3-yl)phenoxy]methyl}quinoline; 2-{[4-(4-pyridin-4-yl-1H-pyrazol-3-yl)phenoxy]methyl}quinoline; 2-{[4-(TRIFLUOROMETHOXY)BENZOYL]AMINO}ETHYL DIHYDROGEN PHOSPHATE; 2-{[Formyl(Hydroxy)Amino]Methyl}-4-Methylpentanoic Acid; 4′-[(1R)-1-amino-2-(2,5-difluorophenyl)ethyl]biphenyl-3-carboxamide; 2-{1-[2-(2-Amino-Thiazol-4-Yl)-2-Methoxyimino-Acetylamino]-2-Oxo-Ethyl}-5,5-Dimethyl-Thiazolidine-4-Carboxylic Acid; 2-{1-[2-Amino-2-(4-Hydroxy-Phenyl)-Acetylamino]-2-Oxo-Ethyl}-5,5-Dimethyl-Thiazolidine-4-Carboxylic Acid; 4-[(3R)-3-{[2-(4-FLUOROPHENYL)-2-OXOETHYL]AMINO}BUTYL]BENZAMIDE; 2-{2-[2-2-(Methoxy-Ethoxy)-Ethoxy]-Ethoxy}-Ethanol; 2-{3-[(2S)-4,4-difluoro-2-(pyrrolidin-1-ylcarbonyl)pyrrolidin-1-yl]-3-oxopropyl}-isoindole-1,3(2H)-dione; 2-{3-[4-(4-Fluorophenyl)-3,6-Dihydro-1(2h)-Pyridinyl]Propyl}-8-Methyl-4(3h)-Quinazolinone; 2-{4-[(2s)-2-[({[(1s)-1-Carboxy-2-Phenylethyl]Amino}Carbonyl)Amino]-3-Oxo-3-(Pentylamino)Propyl]Phenoxy}Malonic Acid; 4-({4-[(4-methoxypyridin-2-yl)amino]piperidin-1-yl}carbonyl)benzonitrile; 2-{4-[(4-imidazo[1,2-a]pyridin-3-ylpyrimidin-2-yl)amino]piperidin-1-yl}-N-methylacetamide; 2-{4-[2-(2-AMINO-4-OXO-4,7-DIHYDRO-3H-PYRROLO[2,3-D]PYRIMIDIN-5-YL)-ETHYL]-BENZOYLAMINO}-3-METHYL-BUTYRIC ACID; 3-O—N-Octanoyl-a-D-Glucopyranosyl-B-D-Fructofuranoside; 2-{4-[4-(4-Chloro-Phenoxy)-Benzenesulfonyl]-Tetrahydro-Pyran-4-Yl}-N-Hydroxy-Acetamide; 3-Phosphate-Adenosine-5′-Phosphate Sulfate; 2-{5-[3-(6-BENZOYL-1-PROPYLNAPHTHALEN-2-YLOXY)PROPOXYL]INDOL-1-YL}ETHANOIC ACID; 2-{5-[3-(7-PROPYL-3-TRIFLUOROMETHYLBENZO[D]ISOXAZOL-6-YLOXY)PROPOXY]INDOL-1-YL}ETHANOIC ACID; 4-(1,3,2-Dioxaborolan-2-Yloxy)Butan-1-Aminium; 3-PYRIDIN-4-YL-2,4-DIHYDRO-INDENO[1,2-.C.] PYRAZOLE; 2-ACETAMIDO-2-DEOXY-BETA-D-GLUCOPYRANOSE(BETA1-4)-2-ACETAMIDO-1,6-ANHYDRO-3-O-[(R)-1-CARBOXYETHYL]-2-DEOXY-BETA-D-GLUCOPYRANOSE-L-ALANYL-GAMMA-D-GLUTAMYL-MESO-DIAMINOPIMELYL-D-ALANINE; 2-Acetamido-2-Deoxy-D-Glucono-1,5-Lactone; 2-Acetyl-3-[(4-Amino-2-Methyl-5-Pyrimidinyl)Methyl]-4-Methyl-5-(4,6,6-Trihydroxy-3,5-Dioxa-4,6-Diphosphahex-1-Yl)Thiazolium Inner Salt P,P′-Dioxide; 2-ACETYLAMINO-4-METHYL-PENTANOIC ACID (1-FORMYL-2-PHENYL-ETHYL)-AMIDE; 2-ACETYLAMINO-4-METHYL-PENTANOIC ACID [1-(1-FORMYL-PENTYLCARBAMOYL)-3-METHYL-BUTYL]-AMIDE; 2-AMINO-1-METHYL-6-PHENYLIMIDAZO[4,5-B]PYRIDINE; 2-Amino-3-(1h-Indol-3-Yl)-Propan-1-Ol; 2-AMINO-3-(1-HYDROPEROXY-1H-INDOL-3-YL)PROPAN-1-OL; 4-(2,5-Dichloro-Thiophen-3-Yl)-Pyrimidin-2-Ylamine; 2-Amino-3-(4-Amino-1h-Indol-3-Yl)Propanoic Acid; 2-Amino-3-(5-Tert-Butyl-3-(Phosphonomethoxy)-4-Isoxazolyl)Propionic Acid; 2-Amino-3-(Cystein-S-Yl)-Isoxazolidin-5-Yl-Acetic Acid; 2-Amino-3-(Diethoxy-Phosphoryloxy)-Propionic Acid; 4-(2,4-Dimethyl-Thiazol-5-Yl)-Pyrimidin-2-Yl]-(4-Trifluoromethyl-Phenyl)-Amine; 2-Amino-3-[5-(Amino-Carboxy-Methyl)-2,3-Dihydro-Isoxazol-3-Ylsulfanyl]-Propionic Acid; 2-Amino-3-Hydroxy-3-Phosphonooxy-Propionic Acid; 2-Amino-4-(4-Amino-Cyclohexa-2,5-Dienyl)-Butyric Acid; 2-AMINO-4-(2,4-DICHLOROPHENYL)-N-ETHYLTHIENO[2,3-D]PYRIMIDINE-6-CARBOXAMIDE; 2-Amino-4-(2-Amino-Ethoxy)-Butyric Acid; 2-Amino-3-Methyl-1-Pyrrolidin-1-YI-Butan-1-One; 2-Amino-4-(Hydroxymethyl-Phosphinyl)Butanoic Acid; 4-(2-Oxo-Hexahydro-Thieno[3,4-D]Imidazol-4-Yl)-Butyricacid; 2-AMINO-4-CHLORO-3-HYDROXYBENZOIC ACID; 2-AMINO-4-FLUORO-5-[(1-METHYL-1H-IMIDAZOL-2-YL)SULFANYL]-N-(1,3-THIAZOL-2-YL)BENZAMIDE; 2-AMINO-4-HYDROXYPYRIMIDINE-5-CARBOXYLIC ACID ETHYL ESTER; 2-amino-5-[3-(1-ethyl-1H-pyrazol-5-yl)-1H-pyrrolo[2,3-b]pyridin-5-yl]-N,N-dimethylbenzamide; 4-(6-HYDROXY-BENZO[D]ISOXAZOL-3-YL)BENZENE-1,3-DIOL; 2-AMINO-6-(3,5-DIMETHYLPHENYL)SULFONYLBENZONITRILE; 2-amino-6-[2-(1H-indol-6-yl)ethyl]pyrimidin-4(3H)-one; 2-Amino-6-Aminomethyl-8-Phenylsulfanylmethyl-3h-Quinazolin-4-One; 2-Amino-7-[2-(2-Hydroxy-1-Hydroxymethyl-Ethylamino)-Ethyl]-1,7-Dihydro-Purin-6-One; 2-amino-7-fluoro-5-oxo-5H-chromeno[2,3-b]pyridine-3-carboxamide; 2-Amino-8-Methylquinazolin-4(3h)-One; 2-amino-N-(4-methyl-1,3-thiazol-2-yl)-5-[(4-methyl-4H-1,2,4-triazol-3-yl)sulfanyl]benzamide; 2-AMINO-N,N-BIS(PHENYLMETHYL)-1,3-OXAZOLE-5-CARBOXAMIDE; 2-Ammoniobut-3-Enoate, 2-Amino-3-Butenoate; 4-(Acetylamino)-3-[(Hydroxyacetyl)Amino]Benzoic Acid; 2-Benzo[1,3]Dioxol-5-Ylmethyl-3-Benzyl-Succinic Acid; 2-ANILINO-6-CYCLOHEXYLMETHOXYPURINE; 2-Butyl-5,6-Dihydro-1h-Imidazo[4,5-D]Pyridazine-4,7-Dione; 4-(1h-Imidazol-4-Yl)-3-(5-Ethyl-2,4-Dihydroxy-Phenyl)-1h-Pyrazole; 2-chloro-4-{[(1R,3Z,7S,7aS)-7-hydroxy-1-(trifluoromethyl)tetrahydro-1H-pyrrolo[1,2-c][1,3]oxazol-3-ylidene]amino}-3-methylbenzonitrile; 2-CHLORO-4-ETHYLAMINO-6-(R(+)-2′-CYANO-4-BUTYLAMINO)-1,3,5-TRIAZINE; 2-CHLORO-4-ETHYLAMINO-6-(S(−)-2′-CYANO-4-BUTYLAMINO)-1,3,5-TRIAZINE; 2-CHLORO-4-ISOPROPYLAMINO-6-ETHYLAMINO-1,3,5-TRIAZINE; 2-CHLORO-5-(3-CHLORO-PHENYL)-6-[(4-CYANO-PHENYL)-(3-METHYL-3H-IMIDAZOL-4-YL)-METHOXYMETHYL]-NICOTINONITRILE; 2-chloro-5-[(1S)-1-hydroxy-3-oxo-2H-isoindol-1-yl]benzenesulfonamide; 4-(Aminosulfonyl)-N-[(2,4-Difluorophenyl)Methyl]-Benzamide; 2-CHLORO-N-[(1R,2R)-1-HYDROXY-2,3-DIHYDRO-1H-INDEN-2-YL]-6H-THIENO[2,3-B]PYRROLE-5-CARBOXAMIDE; 4-(Aminosulfonyl)-N-[(4-Fluorophenyl)Methyl]-Benzamide; 2c-Methyl-D-Erythritol 2,4-Cyclodiphosphate; 2-Deamino-6-Deoxy-6thiophosphite-5′-Phosphate Guanosine; 2-Deazo-6-Thiophosphate Guanosine-5′-Monophosphate; 2-decyl-5,6-dimethoxy-3-methylcyclohexa-2,5-diene-1,4-dione; 2-fluoro-6-{[2-({2-methoxy-4-[(methylsulfonyl)methyl]phenyl}amino)-7H-pyrrolo[2,3-d]pyrimidin-4-yl]amino}benzamide; 2-Deoxy-2-Amino Glucitol-6-Phosphate; 2-Deoxy-2-Fluoro-Alpha-D-Mannosyl Fluoride; 4-(1-Amino-1-Carboxy-Ethyl)-Benzoic Acid; 2′-DEOXY-5-FORMYLCYTIDINE-5-MONOPHOSPHATE; 2′-Deoxycytidine-2′-Deoxyadenosine-3′,5′-Monophosphate; 2-Deoxy-D-Glucitol 6-(E)-Vinylhomophosphonate; 2′-deoxy-N-(naphthalen-1-ylmethyl)guanosine 5′-(dihydrogen phosphate); 2′deoxy-Thymidine-5′-Diphospho-Alpha-D-Glucose; 2′-deoxyuridine 5′-alpha,beta-imido-diphosphate; 2′-Deoxyuridine 5′-Alpha,Beta-Imido-Triphosphate; 2-Dimethylamino-Ethyl-Diphosphate; 2-DEOXY-3,4-BIS-O-[3-(4-HYDROXYPHENYL)PROPANOYL]-L-THREO-PENTARIC ACID; 4-[(Isopropylamino)Methyl]Phenylalanine; 2-fluoro-4-[4-(4-fluorophenyl)-1H-pyrazol-3-yl]pyridine; 2-Deoxy-2,3-Dehydro-N-Acetyl-Neuraminic Acid; 2-HEPTYL-4-HYDROXY QUINOLINE N-OXIDE; 2-Hexyloxy-6-Hydroxymethyl-Tetrahydro-Pyran-3,4,5-Triol; 2-Hexyloxy-6-Hydroxymethyl-Tetrahydro-Pyran-3,5-Diol; 4-[(6-phenyl[1,2,4]triazolo[4,3-b]pyridazin-3-yl)methyl]phenol; 2-Hydroxy-3-Amino-4-Phenyl Butane; 2-Hydroxy-5-({1-[(2-Naphthyloxy)Methyl]-3-Oxoprop-1-Enyl}Amino)Tyrosine; 2-Hydroxy-5-({1-[(4-Methylphenoxy)Methyl]-3-Oxoprop-1-Enyl}Amino)-L-Tyrosine; 2-HYDROXY-5-(2-MERCAPTO-ETHYLSULFAMOYL)-BENZOIC ACID; 2-Hydroxy-5-[4-(2-Hydroxy-Ethyl)-Piperidin-1-Yl]-5-Phenyl-1h-Pyrimidine-4,6-Dione; 2-HYDROXY-5-{[(1E)-2-PHENYLETHYLIDENE]AMINO}-L-TYROSINE; 4-[(5-ISOPROPYL-1,3-THIAZOL-2-YL)AMINO]BENZENESULFONAMIDE; 2-HYDROXYMETHYL-6-OCTYLSULFANYL-TETRAHYDRO-PYRAN-3,4,5-TRIOL; 2-Hydroxymethyl-Pyrrolidine-3,4-Diol; 4-[3-Methylsulfanylanilino]-6,7-Dimethoxyquinazoline; 2-KETO-6-PHOSPHATE-D-GLUCONIC ACID, ALPHA-FURANOSE FORM; 2-MERCAPTO-N-[1,2,3,10-TETRAMETHOXY-9-OXO-5,6,7,9-TETRAHYDRO-BENZO[A]HEPTALEN-7-YL]ACETAMIDE; 2-METHYL-2-(4-{[({4-METHYL-2-[4-(TRIFLUOROMETHYL)PHENYL]-1,3-THIAZOL-5-YL}CARBONYL)AMINO]METHYL}PHENOXY)PROPANOIC ACID; 2-Methyl-3-(2-Aminothiazolo)Propanal; 2-methyl-3,5,7,8-tetrahydro-4H-thiopyrano[4,3-d]pyrimidin-4-one; 2-METHYLCARBAMOYL-3-(4-PHOSPHONOOXY-PHENYL)-CYCLOPROPANECARBOXYLIC ACID; 4-[Hydroxypethyl-[Methyl-Phosphinoyl]]-3-Oxo-Butanoic Acid; 2-METHYLTHIO-N6-ISOPENTENYL-ADENOSINE-5′-MONOPHOSPHATE; 2′-Monophosphoadenosine 5′-Diphosphoribose; 4-Hydroxy-3,5-Dimethyl-5-(2-Methyl-Buta-1,3-Dienyl)-5h-Thiophen-2-One; 541-Carboxy-1-Phosphonooxy-Ethoxyl)-Shikimate-3-Phosphate; 2-NONYL-4-HYDROXYQUINOLINE N-OXIDE; AMIDINOPHENYL)-5-O-(4″-AMIDINOPHENYL)-1,4:3,6-DIANHYDRO-D-SORBITOL; 5-(2,3-dichlorophenyl)-N-(pyridin-4-ylmethyl)-3-thiocyanatopyrazolo[1,5-a]pyrimidin-7-amine; 2′-O-[1-ETHYL-1H-IMIDAZOL)] THYMIDINE-5-MONOPHOSPHATE; 2″-O-[N-(3-(aminopropyl)2-aminoethyl]paromomycin; 2′-O-Acetyl Adenosine-5-Diphosphoribose; 2′-O-Methyl-3′-Methyl-3′-Deoxy-Arabinofuranosyl-Thymine-5′-Phosphate; 2-Oxy-4-Hydroxy-5-(2-Hydrazinopyridine)Phenylalanine; 2-Phenethyl-2,3-Dihydro-Phthalazine-1,4-Dione; 2-Phenyl-1-[4-(2-Piperidin-1-Yl-Ethoxy)-Phenyl]-1,2,3,4-Tetrahydro-Isoquinolin-6-Ol; 2-phenyl-1H-imidazole-4-carboxylic acid; 4-Carboxy-5-(1-Pentyl)Hexylsulfanyl-1,2,3-Triazole; 2-Ribofuranosyl-3-Iodo-2,3-Dihydro-1h-Pyrazolo[3,4-D]Pyrimidin-4-Ylamine; 2-T-BUTYLAMINO-4-ETHYLAMINO-6-METHYLTHIO-S-TRIAZINE; 3-((3-bromo-5-o-tolylpyrazolo[1,5-a]pyrimidin-7-ylamino)methyl)pyridine 1-oxide; 3-({[(1Z)-(2-methoxyphenyl)methylidene]amino}oxy)propanoic acid; 3-({[(3S)-3,4-dihydroxybutyl]oxy}amino)-1H,2′H-2,3′-biindol-2′-one; 3-({[3,5-DIFLUORO-3′-(TRIFLUOROMETHOXY)BIPHENYL-4-YL]AMINO}CARBONYL)THIOPHENE-2-CARBOXYLIC ACID; 4-Diphosphocytidyl-2-C-Methyl-D-Erythritol; 3-({2-[(2-AMINO-6-METHYLPYRIMIDIN-4-YL)ETHYNYL]BENZYL}AMINO)-1,3-OXAZOL-2(3H)-ONE; 4-Hydroxy-1,2,5-Thiadiazole-3-Carboxylic Acid; 3-({3-[(1S,4aS,6S,7S,9S,9aR)-1,6-dimethyl-2-oxodecahydro-6,9-epoxy-4a,7-methanobenzo[7]annulen-1-yl]propanoyl}amino)-2,4-dihydroxybenzoic acid; 3-({4-[(1E)-3-morpholin-4-yl-3-oxoprop-1-en-1-yl]-2,3-bis(trifluoromethyl)phenyl}sulfanyl)aniline; 4′-Deoxy-4′-Acetylyamino-Pyridoxal-5′-Phosphate; 3-({4-[(5-chloro-1,3-benzodioxol-4-yl)amino]pyrimidin-2-yl}amino)benzenesulfonamide; 4-Hydroxy-1,2,5-Oxadiazole-3-Carboxylic Acid; 3-O-Benzyl-6-Hydroxy-2,4-Bis-(4-Hydroxy-Benzyl)-3-Oxo-[1,2,4]-Triazepane-1-Sulfonyl)-Benzonitrile; 4-Hydroxy-3-Methyl Butyl Diphosphate; 3-(10-METHYL-ANTHRACEN-9-YL)-PROPIONIC ACID; 3-(1H-BENZIMIDAZOL-2-YL)-1H-INDAZOLE; 4-Imidazolmethylene-5-Imidazolone Chromophore; 3-(1H-INDOL-3-YL)-4-(1-{2-[(2S)-1-METHYLPYRROLIDINYL]ETHYL}-1H-INDOL-3-YL)-1H-PYRROLE-2,5-DIONE; 4-iodo-acetamido phenylboronic acid; 3-(1H-tetrazol-5-ylamino)cyclohex-2-en-1-one; 3-(1H-tetrazol-5-ylmethyl)-5,6,7,8-tetrahydro[1]benzothieno[2,3-d]pyrimidin-4(3H)-one; 3-(1-NAPHTHYLMETHOXY)PYRIDIN-2-AMINE; 3-(2,6-difluorophenyl)-2-(methylthio)quinazolin-4(3H)-one; 4-HYDROXY-N′-(4-ISOPROPYLBENZYL)BENZOHYDRAZIDE; 3-(2-Aminoethyl)-4-(Aminomethyl)Heptanedioic Acid; 3-(2-aminoquinazolin-6-yl)-1-(3,3-dimethylindolin-6-yl)-4-methylpyridin-2(1H)-one; 4-Oxo-Nicotinamide-Adenine Dinucleotide Phosphate; 5-(2-CHLORO-4-NITROPHENYL)-2-FUROIC ACID; 3-(2-Benzothiazolylthio)-1-Propanesulfonic Acid; 4-ethyl-5-methyl-2-(1H-tetrazol-5-yl)-1,2-dihydro-3H-pyrazol-3-one; 5-(4-Methoxyphenoxy)-2,4-Quinazolinediamine; 5-(6-Amino-9h-Purin-9-Yl)-4-Hydroxytetrahydrofuran-3-YI Dihydrogen Phosphate; 3-(3,5-Dibromo-4-Hydroxy-Benzoyl)-2-Ethyl-Benzofuran-6-Sulfonic Acid Dimethylamide; 3-(3-aminophenyl)-N-(3-chlorophenyl)pyrazolo[1,5-a]pyrimidin-5-amine; 3-(3-FLUORO-4-HYDROXYPHENYL)-7-HYDROXY-1-NAPHTHONITRILE; 5-(5-(4-(4,5-dihydro-2-oxazoly)phenoxy)pentyl)-3-methyl osoxazole; 3-(3-methylbut-2-en-1-yl)-3H-purin-6-amine; 3-(4-Amino-1-Tert-Butyl-1h-Pyrazolo[3,4-D]Pyrimidin-3-Yl)Phenol; 3-(4-Amino-2-Tert-Butyl-5-Methyl-Phenylsulfanyl)-6-Cyclopentyl-4-Hydroxy-6-[2-(4-Hydroxy-Phenyl)-Ethyl]-5,6-Dihydro-Pyran-2-One; 3-(4-Benzenesulfonyl-Thiophene-2-Sulfonylamino)-Phenylboronic Acid; 3-(4-Carbamoyl-1-Carboxy-2-Methylsulfonyl-Buta-1,3-Dienylamino)-Indolizine-2-Carboxylic Acid; 3-(4-CHLOROPHENYL)-5-(METHYLTHIO)-4H-1,2,4-TRIAZOLE; 3-(4-DIETHYLAMINO-2-HYDROXY-PHENYL)-2-METHYL-PROPIONIC ACID; 3-(4-Fluorophenyl)-1-Hydroxy-2-(Pyridin-4-Yl)-1h-Pyrrolo[3,2-B]Pyridine; 3-(4-Fluorophenyl)-2-(6-Methylpyridin-2-Yl)-5,6-Dihydro-4h-Pyrrolo[1,2-B]Pyrazole; 3-(4-fluorophenyl)-5-phenyl-4H-1,2,4-triazole; 3-(4-Hydroxy-3-Imino-6-Oxo-Cyclohexa-1,4-Dienyl)-Alanine; 3-(4-HYDROXY-3-METHOXYPHENYL)-2-PROPENOIC ACID; 3-(4-HYDROXYPHENYL)-1-(2,4,6-TRIHYDROXYPHENYL)PROPAN-1-ONE; 3-(4-Phenylamino-Phenylamino)-2-(1h-Tetrazol-5-Yl)-Acrylonitrile; 3-(5-{[4-(AMINOMETHYL)PIPERIDIN-1-YL]METHYL}-1H-INDOL-2-YL)-1H-INDAZOLE-6-CARBONITRILE; 5-(PARA-NITROPHENYL PHOSPHONATE)-PENTANOIC ACID; 3-(4-HYDROXYPHENYL)-4,5-DIHYDRO-5-ISOXAZOLE-ACETIC ACID METHYL ESTER; 3-(5-Amino-7-Hydroxy-[1,2,3]Triazolo[4,5-D]Pyrimidin-2-Yl)-Benzoic Acid; 3-(5-Amino-7-Hydroxy-[1,2,3]Triazolo[4,5-D]Pyrimidin-2-Yl)-N-(3,5-Dichlorobenzyl)-Benzamide; 5-[(phenylsulfonyl)amino]-1,3,4-thiadiazole-2-sulfonamide; 3-(5-methoxy-1H-indol-3-yl)propanoic acid; 3-(6-Aminopyridin-3-Yl)-N-Methyl-N-[(1-Methyl-1h-Indol-2-Yl)Methyl]Acrylamide; 3-(6-CYCLOHEXYLMETHOXY-9H-PURIN-2-YLAMINO)-BENZENESULFONAMIDE; 3-(6-HYDROXY-NAPHTHALEN-2-YL)-BENZO[D]ISOOXAZOL-6-OL; 5,7-dihydroxy-2-(4-hydroxyphenyl)-4H-chromen-4-one; 3-(7-Hydroxy-8-Ribityllumazine-6-Yl) Propionic Acid; 5-{[1-(2,3-dichlorobenzyl)piperidin-4-yl]methoxy}quinazoline-2,4-diamine; 3″-(Beta-Chloroethyl)-2″,4″-Dioxo-3, 5″-Spiro-Oxazolidino-4-Deacetoxy-Vinblastine; 3-(BUTYLSULPHONYL)-PROPANOIC ACID; 3-(CARBOXYAMIDE(2-CARBOXYAMIDE-2-TERTBUTYLETHYL))PENTAN; 3-(CARBOXYMETHOXY)THIENO[2,3-B]PYRIDINE-2-CARBOXYLIC ACID; 3-(HYDROXYMETHYL)-1-METHYL-5-(2-METHYLAZIRIDIN-1-YL)-2-PHENYL-1H-INDOLE-4,7-DIONE; 3-(HYDROXY-PHENYL-PHOSPHINOYLOXY)-8-METHYL-8-AZA-BICYCLO[3.2.1]OCTANE-2-CARBOXYLIC ACID METHYL ESTER; 5-{[1-(2-fluorobenzyl)piperidin-4-yl]methoxy}quinazoline-2,4-diamine; 3(S)-METHYLCARBAMOYL-7-SULFOAMINO-3,4-DIHYDRO-1H-ISOQUINOLINE-2-CARBOXYLIC ACID TERT-BUTYL ESTER; 3,3-Dichloro-2-Phosphonomethyl-Acrylic Acid; 3,4-bis(7-chloro-1H-indol-3-yl)-1H-pyrrole-2,5-dicarboxylic acid; 3,4-DI-1H-INDOL-3-YL-1H-PYRROLE-2,5-DICARBOXYLIC ACID; 3,4-DIHYDRO-4-OXO-3-((5-TRIFLUOROMETHYL-2-BENZOTHIAZOLYL)METHYL)-1-PHTHALAZINE ACETIC ACID; 3,4-dihydroxy-9,10-secoandrosta-1(10),2,4-triene-9,17-dione; 3,5,6,8-Tetramethyl-N-Methyl Phenanthrolinium; 3′,5′-DIBROMO-2′,4,4′,6′-TETRAHYDROXY AURONE; 3,5-Dichloro-4-[(4-Hydroxy-3-Isopropylphenoxy)Phenylacetic Acid; 3′,5′-DIFLUOROBIPHENYL-4-CARBOXYLIC ACID; 3,5-Dihydro-5-Methylidene-4h-Imidazol-4-On; 3,5-Dimethyl-1-(3-Nitrophenyl)-1h-Pyrazole-4-Carboxylic Acid Ethyl Ester; 5-Amino-3-Methyl-Pyrrolidine-2-Carboxylic Acid; 3,5-DIMETHYL-1-PHENYL-1H-PYRAZOLE-4-CARBOXYLIC ACID ETHYL ESTER; 5-{[Ethyl(Methyl)Amino]Methyl}-2-Methyl-5,6-Dihydropyrimidin-4-Amine; 3,7-BIS(DIMETHYLAMINO)PHENOTHIAZIN-5-IUM; 3,7-DIHYDROXYNAPHTHALENE-2-CARBOXYLIC ACID; 5-Bromo-2′-Deoxyuridine-5-Monophosphate; 3,8-Diamino-6-Phenyl-5-[6-[1-[2-[(1,2,3,4-Tetrahydro-9-Acridinyl)Amino]Ethyl]-1h-1,2,3-Triazol-4-Yl]Hexyl]-Phenanthridinium; 3,8-Diamino-6-Phenyl-5-[6-[1-[2-[(1,2,3,4-Tetrahydro-9-Acridinyl)Amino]Ethyl]-1h-1,2,3-Triazol-5-Yl]Hexyl]-Phenanthridinium; 3,8-DIBROMO-7-HYDROXY-4-METHYL-2H-CHROMEN-2-ONE; 5-Hydroxyamino-3-Methyl-Pyrrolidine-2-Carboxylic Acid; 5-Hydroxymethylene-6-Hydrofolic Acid; 5-Hydroxy-2-(Hydroxymethyl)-4h-Pyran-4-One; 5-Mercapto-2-Nitro-Benzoic Acid; 3-[(2,2-DIMETHYLPROPANOYL)AMINO]-N-1,3-THIAZOL-2-YLPYRIDINE-2-CARBOXAMIDE; 5-Methoxy-N,N-diisopropyltryptamine; 3-[(3-(2-CARBOXYETHYL)-4-METHYLPYRROL-2-YL)METHYLENE]-2-INDOLINONE; 3-[(4-AMINO-1-TERT-BUTYL-1H-PYRAZOLO[3,4-D]PYRIMIDIN-3-YL)METHYL]PHENOL; 5-Methoxy-1,2-Dimethyl-3-(Phenoxymethyl)Indole-4,7-Dione; 3-[(4′-cyanobiphenyl-4-yl)oxy]-N-hydroxypropanamide; 5-methyl-6-phenylquinazoline-2,4-diamine; 3-[(5s)-1-Acetyl-3-(2-Chlorophenyl)-4,5-Dihydro-1h-Pyrazol-5-Yl]Phenol; 3-[(9H-fluoren-9-ylideneamino)oxy]propanoic acid; 3-[(Acetyl-Methyl-Amino)-Methyl]-4-Amino-N-Methyl-N-(1-Methyl-1h-Indol-2-Ylmethyl)-Benzamide; 5-[Bis-2(Chloro-Ethyl)-Amino]-2,4-Dintro-Benzamide; 3-[[N-[4-Methyl-Piperazinyl]Carbonyl]-Phenylalaninyl-Amino]-5-Phenyl-Pentane-1-Sulfonic Acid Benzyloxy-Amide; 3-[1-(3-AMINOPROPYL)-1H-INDOL-3-YL]-4-(1H-INDOL-3-YL)-1H-PYRROLE-2,5-DIONE; 3-[1-(3-Aminopropyl)-1h-Indol-3-Yl]-4-(1-Methyl-1h-Indol-3-Yl)-1h-Pyrrole-2,5-Dione; 3-[1-(4-BROMO-PHENYL)-2-METHYL-PROPYL]-4-HYDROXY-CHROMEN-2-ONE; 3-[2-(2-BENZYLOXYCARBONYLAMINO-3-METHYL-BUTYRYLAMINO)-PROPIONYLAMINO]-4-OXO-PENTANOIC ACID; 3-[2-bromo-4-(1H-pyrazolo[3,4-c]pyridazin-3-ylmethyl)phenoxy]-5-methylbenzonitrile; 3-[3-(2,3-Dihydroxy-Propylamino)-Phenyl]-4-(5-Fluoro-1-Methyl-1h-Indol-3-Yl)-Pyrrole-2,5-Dione; 3-[3-(3-methyl-6-{[(1S)-1-phenylethyl]amino}-1H-pyrazolo[4,3-c]pyridin-1-yl)phenyl]propanamide; 3-[3-(4-methylpiperazin-1-yl)-7-(trifluoromethyl)quinoxalin-5-yl]phenol; 6((S)-3-Benzylpiperazin-1-Yl)-3-(Naphthalen-2-Yl)-4-(Pyridin-4-Yl)Pyrazine; 3-[4-(2,4-Dimethyl-Thiazol-5-Yl)-Pyrimidin-2-Ylamino]-Phenol; 3-[4-(2-METHYL-IMIDAZO[4,5-C]PYRIDIN-1-YL)BENZYL]-3H-BENZOTHIAZOL-2-ONE; 3-[4-(AMINOSULFONYL)PHENYL]PROPANOIC ACID; 3-[4-AMINO-1-(1-METHYLETHYL)-1H-PYRAZOLO[3,4-D]PYRIMIDIN-3-YL]PHENOL; 3-[5-(1H-IMIDAZOL-1-YL)-7-METHYL-1H-BENZIMIDAZOL-2-YL]-4-[(PYRIDIN-2-YLMETHYL)AMINO]PYRIDIN-2(1H)-ONE; 3-[5-(2-nitropent-1-en-1-yl)furan-2-yl]benzoic acid; 3-[5-(3-nitrophenyl)thiophen-2-yl]propanoic acid; 5-O-(N-(L-Cysteinyl)-Sulfamoyl)Adenosine; 3-[Aminoethylphosphoryl]-[1,2-Di-Palmitoyl]-Sn-Glycerol; 5′-O-(N-(L-Seryl)-Sulfamoyl)Adenosine; 3-[N-[Benzyloxycarbonyl]-Phenylalaninyl-Amino]-5-Phenyl-Pentane-1-Sulfonic Acid 4-Nitro-Phenyl Ester; 5-O-(N-Ethyl-Sulfamoyl)Adenosine; 3-{(3R,4R)-4-methyl-3-[methyl(7H-pyrrolo[2,3-d]pyrimidin-4-yl)amino]piperidin-1-yl}-3-oxopropanenitrile; 3-{[(1r)-1-Benzyl-2-Sulfanylethyl]Amino}-3-Oxopropanoic Acid; 5-O-[(L-Methionyl)-Sulphamoyl]Adenosine; 5-Nitro-6-Ribityl-Amino-2,4(1h,3h)-Pyrimidinedione; 3-{[(2,2-DIOXIDO-1,3-DIHYDRO-2-BENZOTHIEN-5-YL)AMINO]METHYLENE}-5-(1,3-OXAZOL-5-YL)-1,3-DIHYDRO-2H-INDOL-2-ONE; 3-{[(3-FLUORO-3-METHOXYBIPHENYL-4-YL)AMINO]CARBONYL}THIOPHENE-2-CARBOXYLIC ACID; 3-{[(3-NITROANILINE]SULFONYL}THIOPHENE-2-CARBOXYLIC ACID; 3-{[(4-CARBOXY-2-HYDROXYANILINE]SULFONYL}THIOPHENE-2-CARBOXYLIC ACID; 6,8-DIMERCAPTO-OCTANOIC ACID AMIDE; 6-(2-phenoxyethoxy)-1,3,5-triazine-2,4-diamine; 5-S-[2-(decylamino)ethyl]-5′-thioadenosine; 3-{[4-(but-2-yn-1-yloxy)phenyl]sulfonyl}propane-1-thiol; 5-Thio-a/B-D-Mannopyranosylamine; 3-{2,6,8-trioxo-9-[(2R,3S,4R)-2,3,4,5-tetrahydroxypentyl]-1,2,3,6,8,9-hexahydro-7H-purin-7-Yl}propyl dihydrogen phosphate; 6-[2-(3′-METHOXYBIPHENYL-3-YL)ETHYL]PYRIDIN-2-AMINE; 3-{2,6,8-trioxo-9-[(2S,3S,4R)-2,3,4,5-tetrahydroxypentyl]-1,2,3,6,8,9-hexahydro-7H-purin-7-Yl}propyl dihydrogen phosphate; 3-{3-[(DIMETHYLAMINO)METHYL]-1H-INDOL-7-YL}PROPAN-1-OL; 3-{3-bromo-4-[(2,4-difluorobenzyl)oxy]-6-methyl-2-oxopyridin-1(2H)-yl}-N,4-dimethylbenzamide; 3-{5-[(6-amino-1H-pyrazolo[3,4-b]pyridin-3-yl)methoxy]-2-chlorophenoxy}-5-chlorobenzonitrile; 3-{5-[AMINO(IMINIO)METHYL]-1H-INDOL-2-YL}-5-METHOXY-1,1′-BIPHENYL-2-OLATE; 3-{5-methoxy-1-[(4-methoxyphenyl)sulfonyl]-1H-indol-3-yl}propanoic acid; 6-[4-(2-piperidin-1-ylethoxy)phenyl]-3-pyridin-4-ylpyrazolo[1,5-a]pyrimidine; 3-{ISOPROPYL[(TRANS-4-METHYLCYCLOHEXYL)CARBONYL]AMINO}-5-PHENYLTHIOPHENE-2-CARBOXYLIC ACID; 3′-1-Carboxy-1-Phosphonooxy-Ethoxy-Uridine-Diphosphate-N-Acetylglucosamine; 3-AMINO-3-BENZYL-[4.3.0]BICYCLO-1,6-DIAZANONAN-2-ONE; 6-BENZYL-1-ETHOXYMETHYL-5-ISOPROPYL URACIL; 3-Amino-4,5-Dihydroxy-Cyclohex-1-Enecarboxylate; 3-Amino-4-{3-[2-(2-Propoxy-Ethoxy)-Ethoxy]-Propylamino}-Cyclobut-3-Ene-1,2-Dione; 3-Amino-8,9,10-Trihydroxy-7-Hydroxymethyl-6-Oxa-1,3-Diaza-Spiro[4.5]Decane-2,4-Dione; 3-AMINO-AZACYCLOTRIDECAN-2-ONE; 6-HYDROXY-1,3-BENZOTHIAZOLE-2-SULFONAMIDE; 3-Amino-N-{4-[2-(2,6-Dimethyl-Phenoxy)-Acetylamino]-3-Hydroxy-1-Isobutyl-5-Phenyl-Pentyl}-Benzamide; 3ar,5r,6s,7r,7ar-5-Hydroxymethyl-2-Methyl-5,6,7,7a-Tetrahydro-3ah-Pyrano[3,2-D]Thiazole-6,7-Diol; 3-Aza-2,3-Dihydrogeranyl Diphosphate; 3-Azido-3-Deoxythymidine-5′-Diphosphate; 3-Azido-3-Deoxythymidine-5-Monophosphate; 3-Benzylaminocarbonylphenyl-Alpha-D-Galactoside; 6-Chloropurine Riboside, 5′-Monophosphate; 6-Hydroxy-7,8-Dihydro Purine Nucleoside; 3-bromo-5-phenyl-N-(pyridin-4-ylmethyl)pyrazolo[1,5-a]pyrimidin-7-amine; 3-bromo-5-phenyl-N-(pyrimidin-5-ylmethyl)pyrazolo[1,5-a]pyridin-7-amine; 3-BROMO-6-HYDROXY-2-(4-HYDROXYPHENYL)-1H-INDEN-1-ONE; 6-Hydroxy-Flavin-Adenine Dinucleotide; 3-bromo-N′-[(1E)-(3,5-dibromo-2,4-dihydroxyphenyl)methylidene]benzohydrazide; 3-CARBOXAMIDO-1,3,5(10)-ESTRATRIEN-17(R)-SPIRO-2′(5′,5′-DIMETHYL-6′OXO)TETRAHYDROPYRAN; 3-CARBOXY-4-METHYL-5-PROPYL-2-FURANPROPIONIC; 3-Carboxy-N,N,N-Trimethyl-2-(Octanoyloxy)Propan-1-Aminium; 3-CHLORO-2-(4-HYDROXYPHENYL)-2H-INDAZOL-5-OL; 3-CHLORO-2,2-DIMETHYL-N-[4-(TRIFLUOROMETHYL)PHENYL]PROPANAMIDE; 3-chloro-5-[2-chloro-5-(1H-indazol-3-ylmethoxy)phenoxy]benzonitrile; 3-chloro-5-[2-chloro-5-(1H-pyrazolo[3,4-b]pyridin-3-ylmethoxy)phenoxy]benzonitrile; 3-Chloro-9-Ethyl-6,7,8,9,10,11-Hexahydro-7,11-Methanocycloocta[B]Quinolin-12-Amine; 7,8-dihydroxy-4-phenyl-2H-chromen-2-one; 3-cyclohexyl-D-alanyl-N-(3-chlorobenzyl)-L-prolinamide; 3-cyclopropyl-5-phenyl-N-(pyridin-3-ylmethyl)pyrazolo[1,5-a]pyrimidin-7-amine; 3-Decyl-2,5-Dioxo-4-Hydroxy-3-Pyrroline; 7-[(3-CHLOROBENZYL)OXY]-4-[(METHYLAMINO)METHYL]-2H-CHROMEN-2-ONE; 3-Deoxy-D-Manno-Oct-2-Ulosonic Acid; 3-DIPHENOL-6-NITRO-3H-BENZO[DE]ISOCHROMEN-1-ONE; 3-ETHYL-2-(4-HYDROXYPHENYL)-2H-INDAZOL-5-OL; 3-ETHYL-6-{[(4-FLUOROPHENYL)SULFONYL]AMINO}-2-METHYLBENZOIC ACID; 7-[2-METHOXY-1-(METHOXYMETHYL)ETHYL]-7H-PYRROLO[3,2-F] QUINAZOLINE-1,3-DIAMINE; 7-Alpha-D-Ribofuranosyl-Purine-5′-Phosphate; 7-amino-2-tert-butyl-4-(4-pyrimidin-2-ylpiperazin-1-yl)pyrido[2,3-d]pyrimidine-6-carboxamide; 3-fluoro-N-[3-(1H-tetrazol-5-yl)phenyl]benzamide; 3-FLUORO-N-[1-(4-FLUOROPHENYL)-3-(2-THIENYL)-1H-PYRAZOL-5-YL]BENZENESULFONAMIDE; 3-FORMYL-2-HYDROXY-5-METHYL-HEXANOIC ACID HYDROXYAMIDE; 7-AMINO-4-METHYL-CHROMEN-2-ONE; 3-Hydroxymethyl-5-Aziridinyl-1methyl-2-[1h-Indole-4,7-Dione]-Propanol; 3-HYDROXYPROPYL 3-[({7-[AMINO(IMINO)METHYL]-1-NAPHTHYL}AMINO)CARBONYL]BENZENESULFONATE; 3-Mercapto-1-(1,3,4,9-Tetrahydro-B-Carbolin-2-Yl)-Propan-1-One; 3-METHOXY-6-[4-(3-METHYLPHENYL)-1-PIPERAZINYL]PYRIDAZINE; 3-Methyl-5-Sulfo-Pyrrolidine-2-Carboxylic Acid; 3-METHYL-1,6,8-TRIHYDROXYANTHRAQUINONE; 3-FLUORO-4-HYDROXYBENZALDEHYDE O-(CYCLOHEXYLCARBONYL)OXIME; 7-Hydroxy-2-Oxo-Chromene-3-Carboxylic Acid Ethyl Ester; 3-FLUORO-5-MORPHOLIN-4-YL-N-[1-(2-PYRIDIN-4-YLETHYL)-1H-INDOL-6-YL]BENZAMIDE; 7n-Methyl-8-Hydroguanosine-5′-Diphosphate; 7-thionicotinamide-adenine-dinucleotide phosphate; 4-({4-[(6-CHLORO-1-BENZOTHIEN-2-YL)SULFONYL]-2-OXOPIPERAZIN-1-YL}METHYL)BENZENECARBOXIMIDAMIDE; 8-(2-Chloro-3,4,5-Trimethoxy-Benzyl)-2-Fluoro-9-Pent-4-Ylnyl-9h-Purin-6-Ylamine; 4-({6-AMINO-5-BROMO-2-[(4-CYANOPHENYL)AMINO]PYRIMIDIN-4-YL}OXY)-3,5-DIMETHYLBENZONITRILE; 4-({(2R,5S)-2,5-DIMETHYL-4-[(2R)-3,3,3-TRIFLUORO-2-HYDROXY-2-METHYLPROPANOYL]PIPERAZIN-1-YL}CARBONYL)BENZONITRILE; 8-(2,5-Dimethoxy-Benzyl)-2-Fluoro-9-Pent-9h-Purin-6-Ylamine; 4-(3,14-Dihydroxy-10,13-Dimethyl-Hexadecahydro-Cyclopenta[a]Phenanthren-17-Yl)-5h-Furan-2-One; 4-(3,5-DIMETHYLPHENOXY)-5-(FURAN-2-YLMETHYLSULFANYLMETHYL)-3-IODO-6-METHYLPYRIDIN-2(1H)-ONE; 4-(3-{[5-(trifluoromethyl)pyridin-2-yl]oxy}benzyl)piperidine-1-carboxylic acid; 8-Amino-1,3-Dimethyl-3,7-Dihydropurine-2,6-Dione; 4,6-Dideoxy-4-{[4,5,6-Trihydroxy-3-(Hydroxymethyl)Cyclohex-2-En-1-Yl]Amino}-Alpha-D-Lyxo-Hexopyranosyl-(1->4)-Alpha-D-Threo-Hexopyranosyl-(1->6)-Alpha-L-Threo-Hexopyranose; 4,6-DIDEOXY-4-{[4-[(4-O-HEXOPYRANOSYLHEXOPYRANOSYL)OXY]-5,6-DIHYDROXY-3-(HYDROXYMETHYL)CYCLOHEX-2-EN-1-YL]AMINO}HEXOPYRANOSYL-(1-&gt;4)HEXOPYRANOSYL-(1-&gt;4)HEXOPYRANOSE; 8-Benzo[1,3]Dioxol-,5-Ylmethyl-9-Butyl-2-Fluoro-9h-Purin-6-Ylamine; 4-[(1S,2S,5S)-5-(HYDROXYMETHYL)-6,8,9-TRIMETHYL-3-OXABICYCLO[3.3.1]NON-7-EN-2-YL]PHENOL; 8-Demethyl-8-Dimethylamino-Flavin-Adenine-Dinucleotide; 4-[(1S,2S,5S,9R)-5-(HYDROXYMETHYL)-8,9-DIMETHYL-3-OXABICYCLO[3.3.1]NON-7-EN-2-YL]PHENOL; 8-Hydroxy-4-(1-Hydroxyethyl)Quinoline-2-Carboxylic Acid; 4-[(3AS,4R,7R,8AS,8BR)-2-(1,3-BENZODIOXOL-5-YLMETHYL)-7-HYDROXY-1,3-DIOXODECAHYDROPYRROLO[3,4-A]PYRROLIZIN-4-YL]BENZENECARBOXIMIDAMIDE; 9-(4-Hydroxyphenyl)-2,7-Phenanthroline; 4-[(3-CHLORO-4-{[(2R)-3,3,3-TRIFLUORO-2-HYDROXY-2-METHYLPROPANOYL]AMINO}PHENYL)SULFONYL]-N,N-DIMETHYLBENZAMIDE; 4-({[(4-METHYLPIPERAZIN-1-YL)AMINO]CARBONOTHIOYL}AMINO)BENZENESULFONAMIDE; 8-Bromo-Adenosine-5′-Monophosphate; 4-[(3-BROMO-4-O-SULFAMOYLBENZYL)(4-CYANOPHENYL)AMINO]-4H-[1,2,4]-TRIAZOLE; 6-(4-chloro-2-fluoro-3-phenoxybenzyl)pyridazin-3(2H)-one; 9-(5,5-Difluoro-5-Phosphonopentyl)Guanine; 6-[3-(4-Morpholinyl)Propyl]-2-(3-Nitrophenyl)-5-Thioxo-5,6,-Dihydro-7h-Thienol[2′,3′:4,5]Pyrrolo[1,2-C]Imidazol-7-One; 9-(6-Deoxy-Beta-D-Allofuranosyl)-6-Methylpurine; 6-[(5-CHLORO-3-METHYL-1-BENZOFURAN-2-YL)SULFONYL]PYRIDAZIN-3(2H)-ONE; 9-benzyl-2,3,4,9-tetrahydro-1H-carbazole-8-carboxylic acid; 9-amino-5-(2-aminopyrimidin-4-yl)pyrido[3′,2′:4,5]pyrrolo[1,2-c]pyrimidin-4-ol; 6-[1-(3,5,5,8,8-Pentamethyl-5,6,7,8-Tetrahydronaphthalen-2-Yl)Cyclopropyl]Pyridine-3-Carboxylic Acid; 6-AMINO-4-[2-(4-METHOXYPHENYL)ETHYL]-1,7-DIHYDRO-8H-IMIDAZO[4,5-G]QUINAZOLIN-8-ONE; 6-AMINO-4-[2-(4-METHYLPHENYL)ETHYL]-1,7-DIHYDRO-8H-IMIDAZO[4,5-G]QUINAZOLIN-8-ONE; 6-BROMO-13-THIA-2,4,8,12,19-PENTAAZATRICYCLO[12.3.1.1˜3,7˜]NONADECA-1(18),3(19),4,6,14,16-HEXAENE 13,13-DIOXIDE; 9-Beta-D-Xylofuranosyl-Adenine; 6-CARBAMIMIDOYL-2-[2-HYDROXY-6-(4-HYDROXY-PHENYL)-INDAN-1-YL]-HEXANOIC ACID; 9-Butyl-8-(2,5-Dimethoxy-Benzyl)-9h-Purin-6-Ylamine; 6-CHLORO-1-(2-{[(5-CHLORO-1-BENZOTHIEN-3-YL)METHYL]AMINO}ETHYL)-3-[(2-PYRIDIN-2-YLETHYL)AMINO]-1,4-DIHYDROPYRAZIN-2-OL; 6-CHLORO-2-(1-FURO[2,3-C]PYRIDIN-5-YL-ETHYLSULFANYL)-PYRIMIDIN-4-YLAMINE; 6,11-DIHYDRO-11-ETHYL-6-METHYL-9-NITRO-5H-PYRIDO[2,3-B][1,5]BENZODIAZEPIN-5-ONE; 6-CHLORO-9-HYDROXY-1,3-DIMETHYL-1,9-DIHYDRO-4H-PYRAZOLO[3,4-B]QUINOLIN-4-ONE; 6-CHLORO-N-{(3S)-1-[(1S)-1-METHYL-2-(4-MORPHOLINYL)-2-OXO ETHYL]-2-OXO-3-PYRROLIDINYL}-2-NAPHTHALENESULFONAMIDE; 4-[(7R,7AS)-7-HYDROXY-1,3-DIOXOTETRAHYDRO-1H-PYRROLO[1,2-C]]IMIDAZOL-2(3H)-YL]-1-NAPHTHONITRILE; 6-FLUORO-2-(2-HYDROXY-3-ISOBUTOXY-PHENYL)-1H-BENZOIMIDAZOLE-5-CARBOXAMIDINE; 9-amino-n-[3-(dimethylamino)propyl]acridine-4-carboxamide; 4-{[4-(1-CYCLOPROPYL-2-METHYL-1H-IMIDAZOL-5-YL)PYRIMIDIN-2-YL]AMINO}-N-METHYLBENZENESULFONAMIDE; 6-CHLORO-3-(DICHLOROMETHYL)-3,4-DIHYDRO-2H-1,2,4-BENZOTHIADIAZINE-7-SULFONAMIDE 1,1-DIOXIDE; 6-CHLORO-4-(CYCLOHEXYLOXY)-3-ISOPROPYLQUINOLIN-2(1H)-ONE; 6-CHLORO-4-(CYCLOHEXYLOXY)-3-PROPYLQUINOLIN-2(1H)-ONE; 6-CHLORO-4-(CYCLOHEXYLSULFANYL)-3-PROPYLQUINOLIN-2(1H)-ONE; 6-METHYL-2(PROPANE-1-SULFONYL)-2H-THIENO[3,2-1N1,2,3]DIAZABORININ-1-OL; 4-[(10s,14s,18s)-18-(2-Amino-2-Oxoethyl)-14-(1-Naphthylmethyl)-8,17,20-Trioxo-7,16,19-Triazaspiro[5.14]Icos-11-En-10-Yl]Benzylphosphonic Acid; 4-[(1E,7E)-8-(2,6-DIOXO-1,2,3,6-TETRAHYDROPYRIMIDIN-4-YL)-3,6-DIOXA-2,7-DIAZAOCTA-1,7-DIEN-1-YL]BENZOIC ACID; 4-[[(1E)-2-(4-CHLOROPHENYL)ETHENYL]SULFONYL]-1-[[1-(4-PYRIDINYL)-4-PIPERIDINYL]METHYL]PIPERAZINONE; ((2r,3s,5r)-3-Hydroxy-5-(4-Hydroxy-2-Oxo-3,4-Dihydropyrimidin-1(2h)-Yl)-Tetrahydrofuran-2-Yl)Methyldihydrogen Phosphate; (10ALPHA,13ALPHA,14BETA,17ALPHA)-17-HYDROXYANDROST-4-EN-3-ONE; 9-Butyl-8-(3-Methoxybenzyl)-9h-Purin-6-Amine; (1-HYDROXY-1-PHOSPHONO-2-[1,1′;3,1″]TERPHENYL-3-YL-ETHYL)-PHOSPHONIC ACID; (1r)-4-[(1e,3e,5e,7z,9e,11z,13e,15e)-17-Hydroxy-3,7,12,16-Tetramethylheptadeca-1,3,5,7,9,11,13,15-Octaen-1-Yl]-3,5,5-Trimethylcyclohex-3-En-1-Ol; (13R,15S)-13-METHYL-16-OXA-8,9,12,22,24-PENTAAZAHEXACYCLO[15.6.2.16,9.1,12,15.0,2,7.0,21,25]HEPTACOSA-1(24),2,4,6,17(25),18,20-HEPTAENE-23,26-DIONE; 9-Hydroxy-8-Methoxy-6-Nitro-Phenanthrol[3,4-D][1,3]Dioxole-5-Carboxylic Acid; A Disubstituted Succinyl Caprolactam Hydroxymate Mmp3inhibitor; ABX-PTH (human monoclonal antibody to parathyroid hormone); AC-(D)PHE-PRO-BOROHOMOLYS-OH; (1R)-2-[(CYANOMETHYL)AMINO]-1-({[2-(DIFLUOROMETHOXY)BENZYL]SULFONYL}METHYL)-2-OXOETHYL MORPHOLINE-4-CARBOXYLATE; AC-(D)PH E-PRO-BOROLYS-OH; (1n)-4-N-Butoxyphenylsulfonyl-(2r)-N-Hydroxycarboxamido-(4s)-Methanesulfonylamino-Pyrrolidine; Adenosine-5′-Monophosphate Glucopyranosyl-Monophosphate Ester; Adenosine-5′-Diphosphate Monothiophosphate; (1R)-1-(2-THIENYLACETYLAMINO)-1-(3-CARBOXYPHENYL)METHYLBORONIC ACID; (1R,2R)—N-(2-AMINOETHYL)-2-{[(4-M ETHOXYPHENYL)SULFONYL]METHYL}CYCLOHEXANECARBOXAMIDE; Adenosine-5-Rp-Alpha-Thio-Triphosphate; (1R,3R)-5-[(2E)-3-{(1S,3R)-2,2,3-trimethyl-3-[6,6,6-trifluoro-5-hydroxy-5-(trifluoromethyl)hex-3-yn-1-yl]cyclopentyl}prop-2-en-1-ylidene]cyclohexane-1,3-diol; (1R)-2-METHYL-1-(PHENYLMETHYL)PROPYL[(1S)-1-FORMYLPENTYL]CARBAMATE; Alpha,Beta-Methyleneadenosine-5′-Triphosphate; (1E)-5-(1-piperidin-4-yl-3-pyridin-4-yl-1H-pyrazol-4-yl)-2,3-dihydro-1H-inden-1-one oxime; Alpha-Adenosine Monophosphate; Alpha-Amino-2-Indanacetic Acid; (1R,2R,3R,4S,5R)-4-(BENZYLAMINO)-5-(METHYLTHIO)CYCLOPENTANE-1,2,3-TRIOL; Alpha-Aminoisobutyric Acid; Alpha-Benzyl-Aminobenzyl-Phosphonic Acid; (1R,3S,5S,8R)-8-HYDROXY-2-OXABICYCLO[3.3.1]NON-6-ENE-3,5-DICARBOXYLIC ACID; (1S)-1-CYCLOPROPYL-2-[(2S)-4-(2,5-DIFLUOROPHENYL)-2-PHENYL-2,5-DIHYDRO-1H-PYRROL-1-YL]-2-OXOETHANAMINE; (1s)-1-(9-Deazaadenin-9-Yl)-1,4,5-Trideoxy-1,4-Imino-5-Methylthio-D-Ribitol; Alpha-D-Glucose 1,6-Bisphosphate; alpha-D-glucose 6-phosphate; Alpha-D-Glucose-1-Phosphate; Alpha-D-Glucose-1-Phosphate-6-Vanadate; (1S)-2-(1H-INDOL-3-YL)-1-[({5-[(E)-2-PYRIDIN-4-YLVINYL]PYRIDIN-3-YL}OXY)METHYL]ETHYLAMINE; (1s)-1(9-Deazahypoxanthin-9yl)1,4-Dideoxy-1,4-Imino-D-Ribitol-5-Phosphate; (1S)-2-[(2S,5R)-2-(AMINOMETHYL)-5-ETHYNYLPYRROLIDIN-1-YL]-1-CYCLOPENTYL-2-OXOETHANAMINE; Alpha-Hydroxy-Beta-Phenyl-Propionic Acid; (1S,2R,5S)-5-[3-(TRIFLUOROMETHYL)-5,6-DIHYDRO[1,2,4]TRIAZOLO[4,3-A]PYRAZIN-7(8H)-YL]-2-(2,4,5-TRIFLUOROPHENYL)CYCLOHEXANAMINE; Alpha-Methylene Adenosine Monophosphate; (1S,4S,5S)-1,4,5-TRIHYDROXY-3-[3-(PHENYLTHIO)PHENYL]CYCLOHEX-2-ENE-1-CARBOXYLIC ACID; (1S,7S,8S,8AR)-1,2,3,7,8,8A-HEXAHYDRO-7-METHYL-8-[2-[(2R,4R)-TETRAHYDRO-4-HYDROXY-6-OXO-2H-PYRAN-2-YL]ETHYL]-1-NAPHTHALENOL; Alpha-methyltryptamine; alpha-N-Acetyl-D-galactosamine; (3AALPHA,4ALPHA,7ALPHA,7AALPHA)-3A,4,7,7A-TETRAHYDRO-2-(4-NITRO-1-NAPHTHALENYL)-4,7-ETHANO-1H-ISOINDOLE-1,3(2H)-DIONE; Alpha-Phosphoribosylpyrophosphoric Acid; (3AR,6R,6AS)-6-((S)-((S)-CYCLOHEX-2-ENYL)(HYDROXY)METHYL)-6A-METHYL-4-OXO-HEXAHYDRO-2H-FURO[3,2-C]PYRROLE-6-CARBALDEHYDE; (3aS)-3a-hydroxy-7-methyl-1-phenyl-1,2,3,3a-tetrahydro-4H-pyrrolo[2,3-b]quinolin-4-one; Aminodi(Ethyloxy)Ethylaminocarbonylbenzenesulfonamide; (3aS,4R,9bR)-2,2-difluoro-4-(4-hydroxyphenyl)-6-(methoxymethyl)-1,2,3,3a,4,9b-hexahydrocyclopenta[c]chromen-8-ol; ANILINOMETHYL GLUCO-PHENYLIMIDAZOLE; (3AS,4R,9BR)-4-(4-HYDROXYPHENYL)-1,2,3,3A,4,9B-HEXAHYDROCYCLOPENTA[C]CHROMEN-9-OL; anti-alpha5Beta1-integrin antibody; Antihemophilic Factor (Recombinant); Antihemophilic Factor (Recombinant), PEGylated; (3AS,4R,9BR)-4-(4-HYDROXYPHENYL)-6-(METHOXYMETHYL)-1,2,3,3A,4,9B-HEXAHYDROCYCLOPENTA[C]CHROMEN-8-OL; autologous adipose stem cell therapy; (1S,3R,4S,5S,7S)-4-{[2-(4-METHOXYPHENOXY)-2-METHYLPROPANOYL]AMINO}ADAMANTAN E-1-CARBOXAMIDE; autologous fibroblast transplant; BENZOYL-TYROSINE-ALANINE-METHYL KETONE; (1S,3S,5S)-2-{(2S)-2-amino-2-[(1R,3S,5R,7S)-3-hydroxytricyclo[3.3.1.1˜3,7˜]dec-1-yl]acetyl}-2-azabicyclo[3.1.0]hexane-3-carbonitrile; Beta-Dadf, Msa, Multisubstrate Adduct Inhibitor; (20S)-19,20,21,22-TETRAHYDRO-19-OXO-5H-18,20-ETHANO-12,14-ETHENO-6,10-METHENO-18H-BENZ[D]IMIDAZO[4,3-K][1,6,9,12]OXATRIAZA-CYCLOOCTADECOSINE-9-CARBONITRILE; Bis(5-Amidino-2-Benzimidazolyl)Methane Ketone Hydrate; (21S)-1AZA-4,4-DIMETHYL-6,19-DIOXA-2,3,7,20-TETRAOXOBICYCLO[19.4.0] PENTACOSANE; beta-D-galactopyranosyl-(1->4)-beta-D-galactopyranose; (2R)-({4-[AMINO(IMINO)METHYL]PHENYL}AMINO){5-ETHOXY-2-FLUORO-3-[(3R)-TETRAHYDROFURAN-3-YLOXY]PHENYL}ACETICACID; beta-phenyl-D-phenylalanyl-N-propyl-L-prolinamide; (2R)-2-(5-CHLORO-2-THIENYL)-N-{(3S)-1-[(1S)-1-METHYL-2-MORPHOLIN-4-YL-2-OXOETHYL]-2-OXOPYRROLIDIN-3-YL}PROPENE-1-SULFONAMIDE; BIPHENYL-4-YL-ACETALDEHYDE; (2R)-1-{4-[(4-ANILINO-5-BROMOPYRIMIDIN-2-YL)AMINO]PHENOXY}-3-(DIMETHYLAMINO)PROPAN-2-OL; bis(4-nitrophenyl) hydrogen phosphate; (2R)-2-{(1R)-2-oxo-1-[(2-thienylacetyl)amino]ethyl}-5,6-dihydro-2h-1,3-thiazine-4-carboxylic acid; beta-D-fructofuranose 1,6-bisphosphate; Bis(5-Amidino-2-Benzimidazolyl)Methanone; (2R)-2-{[(4-FLUORO-3-METHYLPHENYL)SULFONYL]AMINO}-N-HYDROXY-2-TETRAHYDRO-2H-PYRAN-4-YLACETAMIDE; Bis(5-Amidino-Benzimidazolyl)Methane Zinc; Bis(5-Amidino-Benzimidazolyl)Methanone Zinc; (1S,6BR,9AS,11R,11BR)-9A,11B-DIMETHYL-1-[(METHYLOXY)METHYL]-3,6,9-TRIOXO-1,6,6B,7,8,9,9A,10,11,11B-DECAHYDRO-3H-FURO[4,3,2-DE]INDENO[4,5-H][2]BENZOPYRAN-11-YL ACETATE; (2R)-3-{[{[(2S)-2,3-DIHYDROXYPROPYL]OXY}(HYDROXY)PHOSPHORYL]OXY}-2-[(9E)-HEXADEC-9-ENOYLOXY]PROPYL (9E)-OCTADEC-9-ENOATE; (2S)-4-METHYL-2-(3-PHENYLTHIOUREDO)-N-((3S)-TETRAHYDRO-2-HYDROXY-3-FURANYL)PENTANAMIDE; bis(molybdopterin)tungsten cofactor; (2S)-2-[3-(AMINOMETHYL)PH ENYL]-3-[(R)-[(1R)-1-{[(BENZYLOXY)CARBONYL]AMINO}-2-METHYLPROPYL](HYDROXY)PHOSPHORYL]PROPANOIC ACID; C-(1-Azido-Alpha-D-Glucopyranosyl) Formamide; (2S)-2-[3-(AMINOMETHYL)PHENYL]-3-{(R)-HYDROXY[(1R)-2-METHYL-1-{[(3-PHENYLPROPYL)SULFONYL]AMINO}PROPYL]PHOSPHORYL}PROPANOIC ACID; (1-HYDROXY-1-PHOSPHONO-2-[1,1′;4,1″]TERPHENYL-3-YL-ETHYL)-PHOSPHONIC ACID; (2S)-4-(2,5-DIFLUOROPHENYL)-N-METHYL-2-PHENYL-N-PIPERIDIN-4-YL-2,5-DIHYDRO-1H-PYRROLE-1-CARBOXAMIDE; C-1027 Aromatized Chromophore; C16-Fatty-Acyl-Substrate-Mimic; C1-INH (human pasteurized C1-inhibitor concentrate); Carboxymethylthio-3-(3-Chlorophenyl)-1,2,4-Oxadiazol; Carbobenzoxy-Pro-Lys-Phe-Y(Po2)-Aa-Pro-Ome; (1S)-2-{[{[(2S)-2,3-DIHYDROXYPROPYL]OXY}(HYDROXY)PHOSPHORYL]OXY}-1-[(PENTANOYLOXY)METHYL]ETHYL OCTANOATE; CBZ-LEU-LEU-TYR-CH2F; (2R)—N-[(2R)-2-(D1HYDROXYBORYL)-1-L-PROLYLPYRROLIDIN-2-YL]-N-[(5R)-5-(DIHYDROXYBORYL)-1-L-PROLYLPYRROLIDIN-2-YL]-L-PROLINAMIDE; (2R)-2-{[4-(benzylamino)-8-(1-methylethyl)pyrazolo[1,5-a][1,3,5]triazin-2-yl]amino}butan-1-ol; Chorionic Gonadotropin (Human); (2R,4S)-2-[(R)-BENZYLCARBAMOYL- PHENYLACETYL-METHYL]-5,5-DIMETHYL-THIAZOLIDINE-4-CARBOXYLIC ACID; CIS-(1R,2S)-2-AMINO-1,2,3,4-TETRAHYDRONAPHTHALEN-1-OL; (2S)-({(5Z)-5-[(5-ETHYL-2-FURYL)METHYLENE]-4-OXO-4,5-DIHYDRO-1,3-THIAZOL-2-YL}AMINO)(4-FLUOROPHENYL)ACETIC ACID; Co(Iii)-(Deuteroporphyrin Ix); (2R)-N˜4˜-hydroxy-2-(3-hydroxybenzyl)-N˜1˜-[(1S,2R)-2-hydroxy-2,3-dihydro-1H-inden-1-yl]butanediamide; Coagulation factor VIIa Recombinant Human; Coagulation Factor XIII A-Subunit (Recombinant); (2E)-3-{3-[(5-ETHYL-3-IODO-6-METHYL-2-OXO-1,2-DIHYDROPYRIDIN-4-YL)OXY]PHENYL}ACRYLONITRILE; (2E)-N-{4-[(3-bromophenyl)amino]quinazolin-6-yl}-4-(dimethylamino)but-2-enamide; (2S)-1-[4-({6-[(2,6-DIFLUOROPHENYL)AMINO]PYRIMIDIN-4-YL}AMINO)PHENOXY]-3-(DIMETHYLAMINO)PROPAN-2-OL; Cyclic Adenosine Monophosphate; Cyclic Guanosine Monophosphate; Cyclohexyl-Hexyl-Beta-D-Maltoside; Cysteine-Methylene-Carbamoyl-1,10-Phenanthroline; (2S)-2-(4-{[3-CHLORO-5-(TRIFLUOROMETHYL)PYRIDIN-2-YL]OXY}PHENOXY)PROPANOIC ACID; D-[(Amino)Carbonyl]Phenylalanine; (2S)-1-{[5-(3-METHYL-1H-INDAZOL-5-YL)PYRIDIN-3-YL]OXY}-3-PHENYLPROPAN-2-AMINE; D-1-(4-CHLOROPHENYL)-2-(ACETAMIDO)ETHANE BORONIC ACID; D-2-Amino-3-Phosphono-Propionic Acid; D-1-NAPHTHYL-2-ACETAMIDO-ETHANE BORONIC ACID; DECYL(DIMETHYL)PHOSPHINE OXIDE; (2S)-2-(3-bromophenyl)-3-(5-chloro-2-hydroxyphenyl)-1,3-thiazolidin-4-one; DEHYDRO-2(S)-AMINO-6-BORONOHEXANOIC ACID; Delta-(L-Alpha-Aminoadipoyl)-L-Cysteinyl-D-Vinylglycine; Delta-2-Albomycin A1; (2R)-2-(4-CHLOROPHENYL)-2-[4-(1H-PYRAZOL-4-YL)PHENYL]ETHANAMINE; Delta-Bis(2,2′-Bipyridine)Imidazole Osmium (Ii); (2S)-2-[(2,1,3-BENZOTHIADIAZOL-4-YLSULFONYL)AMINO]-2-PHENYL-N-PYRIDIN-4-YLACETAMIDE; D-ERITHRO-2,3-DIAMINO-BUTYRIC ACID; DERIVATIVE OF AKLANONIC ACID METHYL ESTER (AAME); D-Gluco-2,5-Anhydro-1-Deoxy-1-Phosphonohexitol-6-Phosphate; D-Glycero-D-Mannopyranose-7-Phosphate; diethyl [(1R)-1,5-diaminopentyl]boronate; (2S,3S)-3-{3-[4-(METHYLSULFONYL)PHENYL]-1,2,4-OXADIAZOL-5-YL}-1-OXO-1-PYRROLIDIN-1-YLBUTAN-2-AMINE; Diethyl 4-Methylbenzylphosphonate; (2s,4r)-1-Acetyl-N-[(1s)-4-[(Aminoiminomethyl)Amino]-1-(2-Benzothiazolylcarbonyl)Butyl]-4-Hydroxy-2-Pyrrolidinecarboxamide; Di-Stearoyl-3-Sn-Phosphatidylcholine; (2S,5R,8S,11R,12S,15S,18S,19S,E)-8-ISOBUTYL-18-((5S,6S)-6-METHOXY-3,5-DIMETHYL-7-PHENYLHEPTYL)-1,2,5,12,15,19-HEXAMETHYL-3,6,9,13,16,20,25-HEPTAOXO-1,4,7,10,14,17,21-HEPTAAZACYCLOPENTACOS-21-ENE-11,22-DICARBOXYLIC ACID; D-leucyl-N-(3-chlorobenzyl)-L-prolinamide; (2S,3S,4E,6E,8S,9S)-3-amino-9-methoxy-2,6,8-trimethyl-10-phenyldeca-4,6-dienoic acid; D-Myo-Inositol-1,4,5-Triphosphate; (25,45,5R)-2-ISOBUTYL-5-(2-THIENYL)-1-[4-(TRIFLUOROMETHYL)BENZOYL]PYRROLIDINE-2,4-DICARBOXYLIC ACID; (4-{4-[(TERT-BUTOXYCARBONYL)AMINO]-2,2-BIS(ETHOXYCARBONYL)BUTYL}PHENYL)SULFAMIC ACID; D-Myo-Inositol-Hexasulphate; (4ar,6s,8ar)-11-[8-(1,3-Dioxo-1,3-Dihydro-2h-Isoindol-2-Yl)Octyl]-6-Hydroxy-3-Methoxy-5,6,9,10-Tetrahydro-4ah-[1]Benzofuro[3a,3,2-Ef][2]Benzazepin-11-lum; Dodecanesulfonate ion; D-Para-Chlorophenyl-1-Acetamidoboronic Acid Alanine; D-Para-Chlorophenyl-1-Acteamidoboronic Acid Alanine; D-phenylalanyl-N-(3-chlorobenzyl)-L-prolinamide; (2s,5r,6r)-6-{[(6r)-6-(Glycylamino)-7-Oxido-7-Oxoheptanoyl]Amino}-3,3-Dimethyl-7-Oxo-4-Thia-1-Azabicyclo[3.2.0]Heptane-2-Carboxylate; D-phenylalanyl-N-(3-methylbenzyl)-L-prolinamide; (2S,4S,5R)-1-(4-TERT-BUTYLBENZOYL)-2-ISOBUTYL-5-(1,3-THIAZOL-2-YL)PYRROLIDINE-2,4-DICARBOXYLIC ACID; D-phenylalanyl-N-benzyl-L-prolinamide; (2S)—N-[(3S)-1-(2-AMINO-2-OXOETHYL)-2-OXO-1,2,3,4-TETRAHYDROQUINOLIN-3-YL]-2-CHLORO-2H-THIENO[2,3-B]PYRROLE-5-CARBOXAMIDE; (3R)-3-(FLUOROMETHYL)-7-(THIOMORPHOLIN-4-YLSULFONYL)-1,2,3,4-TETRAHYDROISOQUINOLINE; ETHYL HYDROGEN DIETHYLAMIDOPHOSPHATE; (3R)-3-(FLUOROMETHYL)-N-(3,3,3-TRIFLUOROPROPYL)-1,2,3,4-TETRAHYDROISOQUINOLINE-7-SULFONAMIDE; (2S)-N-[(3Z)-5-CYCLOPROPYL-3H-PYRAZOL-3-YLIDENE]-2-[4-(2-OXOIMIDAZOLIDIN-1-YL)PHENYL]PROPANAMIDE; GLYCYLALANYL-N-2-NAPHTHYL-L-PROLINEAMIDE; Flavin-N7 Protonated-Adenine Dinucleotide; (2R,6S)-6-{[methyl(3,4,5-trimethoxyphenyl)amino]methyl}-1,2,5,6,7,8-hexahydroquinazoline-2,4-diamine; (2S,3S)-3-FORMYL-2-({[(4-NITROPHENYL)SULFONYL]AMINO}METHYL)PENTANOIC ACID; Glycyl-L-Alpha-Amino-Epsilon-Pimelyl-D-Alanyl-D-Alanine; Guanosine 5′-(Trihydrogen Diphosphate), P′-D-Mannopyranosyl Ester; Guanosine 5′-Diphosphate Monophosphate; (2E)-2-({(2S)-2-CARBOXY-2-[(PHENOXYACETYL)AMINO]ETHOXY}IMINO)PENTANEDIOIC ACID; GUANOSINE-2′,3-O-METHYLIDENEPHOSPHONATE; HEXYLPHOSPHONIC ACID (R)-2-METHYL-3-PHENYLPROPYL ESTER; HEXYLPHOSPHONIC ACID (S)-2-METHYL-3-PHENYLPROPYL ESTER; (2e,3s)-3-Hydroxy-5′-[(4-Hydroxypiperidin-1-Yl)Sulfonyl]-3-Methyl-1,3-Dihydro-2,3′-Biindol-2′(1′ h)-One; HONH-BENZYLMALONYL-L-ALANYLGLYCINE-P-NITROANILIDE; Hydroxy-Phenyl-Acetic Acid 8-Methyl-8-Aza-Bicyclo[3.2.1]Oct-3-Yl Ester; IMIDAZO[2,1-A]ISOQUINOLINE-2-CARBOHYDRAZIDE; L-1-(4-CHLOROPHENYL)-2-(ACETAMIDO)ETHANE BORONIC ACID; L-1-NAPHTHYL-2-ACETAMIDO-ETHANE BORONIC ACID; L-alanyl-N-[(1S,2R)-1-benzyl-2-hydroxypropyl]-L-alaninamide; (17beta)-17-(cyanomethyl)-2-methoxyestra-1(10),2,4-trien-3-ylsulfamate; L-Alpha-Glycerophospho-D-Myo-Inositol-4,5-Bis-Phosphate; L-Alpha-Glycerophosphorylethanolamine; L-Alpha-Phosphatidyl-Beta-Oleoyl-Gamma-Palmitoyl-Phosphatidylethanolamine; Lambda-Bis(2,2′-Bipyridine)-(5-Methyl-2-2′-Bipyridine)-C9-Adamantane Ruthenium (Ii); Lambda-Bis(2,2′-Bipyridine)Imidazole Osmium (Ii); (1-HYDROXYHEPTANE-1,1-DIYL)BIS(PHOSPHONIC ACID); LCP-AtorFen (atorvastatin and and fenofibrate); L-PROLINE, 1-[(2S)-3-MERCAPTO-2-METHYL-1-OXOPROPYL]-4-(PHENYLTHIO)-, 4S; L-D-(a-Aminoadipoyl)-L-Cysteinyl-D-Valine; L-N(Omega)-Nitroarginine-(4r)-Amino-L-Proline Amide; L-N(Omega)-Nitroarginine-2,4-L-Diaminobutyric Amide; L-Naphthyl-1-Acetamido Boronic Acid Alanine; L-CYSTEIN-S-1-(IMINOMETHYL)-L-ORNITHINE; M-(N,N,N-Trimethylammonio)-2,2,2-Trifluoro-1,1-Dihydroxyethylbenzene; Maltosyl-Alpha (1,4)-D-Gluconhydroximo-1,5-Lactam; (1S,2R)-2-[(2,5-difluorophenyl)carbamoyl]cyclopropanecarboxylic acid; Methoxy polyethylene glycol-epoetin beta; methyl (3S)-3-[(tert-butoxycarbonyl)amino]-4-oxopentanoate; methyl 4-(2,3-dihydroxy-5-methylphenoxy)-2-hydroxy-6-methylbenzoate; Methyl 4,6-O-[(1r)-1-Carboxyethylidene]-Beta-D-Galactopyranoside; methyl 4-bromo-N-[8-(hydroxyamino)-8-oxooctanoyl]-L-phenylalaninate; Methyl(6s)-1-Thio-L-Manno-Hexodialdo-6,2-Pyranoside; Methyl-[4-(4-Piperidine-1-Ylmethyl-Phenyl)-Cyclohexyl]-Carbaminic Acid-(4-Chlorophenyl)-Ester; Methyl-2-S-(Alpha-D-Mannopyranosyl)-2-Thio-Alpha-D-Mannopyranoside; METHYLAMINO-PHENYLALANYL-LEUCYL-HYDROXAMIC ACID; Mixed Carbamic Phosphoric Acid Anhydride of 7,8-Diaminononanic Acid; (2r,3r,4s,5r)-2-Acetamido-3,4-Dihydroxy-5-Hydroxymethyl-Piperidinium; N-(1-Carboxy-3-Phenylpropyl)Phenylalanyl-Alpha-Asparagine; N-(2,3,4,5,6-Pentaflouro-Benzyl)-4-Sulfamoyl-Benzamide; N-(2,3-DIFLUORO-BENZYL)-4-SULFAMOYL-BENZAMIDE; N-(1-PHENYL-PROPYL)-FORMAMIDE; N-(2,6-dimethylphenyl)-5-phenylimidazo[1,5-a]pyrazin-8-amine; (2r)-2-{[Formyl(Hydroxy)Amino]Methyl}Hexanoic Acid; (2R)-2-({6-[benzyl(methyl)amino]-9-isopropyl-9H-purin-2-yl}amino)butan-1-ol; N-(2-Aminoethyl)-5-Chloroisoquinoline-8-Sulfonamide; N-(2-AMINOETHYL)-P-CHLOROBENZAMIDE; N-(2-chloro-6-methylphenyl)-8-[(3S)-3-methylpiperazin-1-yl]imidazo[1,5-a]quinoxalin-4-amine; N-(2-chlorophenyl)-5-phenylimidazo[1,5-a]pyrazin-8-amine; N-(2-Ferrocenylethyl)Maleimide; N-(2-Flouro-Benzyl)-4-Sulfamoyl-Benzamide; N-(2-OXOTETRAHYDROFURAN-3-YL)OCTANAMIDE; N′-(2s,3r)-3-Amino-4-Cyclohexyl-2-Hydroxy-Butano-N-(4-Methylphenyl)Hydrazide; N-(2-Thienylmethyl)-2,5-Thiophenedisulfonamide; N-(3,5-dibromo-4-hydroxyphenyl)-2,6-dimethylbenzamide; N-(3-(Aminomethyl)Benzyl)Acetamidine; N-(3,5-dibromo-4-hydroxyphenyl)-4-hydroxy-3,5-dimethylbenzamide; (2S)-1,3-benzothiazol-2-yl{2-[(2-pyridin-3-ylethyl)amino]pyrimidin-4-yl}ethanenitrile; N-(3,5-dimethoxyphenyl)imidodicarbonimidic diamide; (2S)-2-(BUTYRYLOXY)-3-HYDROXYPROPYL NONANOATE; N-(3-carboxypropanoyl)-L-norvaline; N-(3-chlorobenzyl)-1-(4-methylpentanoyl)-L-prolinamide; N-(3-cyano-4,5,6,7-tetrahydro-1-benzothien-2-yl)-2-fluorobenzamide; N-(3-cyclopropyl-1H-pyrazol-5-yl)-2-(2-naphthyl)acetamide; (2S)-2-ETHOXY-3-{4-[2-(10H-PHENOXAZIN-10-YL)ETHOXY]PHENYL}PROPANOIC ACID; N-(3-METHYLBUT-2-EN-1-YL)-9H-PURIN-6-AMINE; N-(3-Phenyl-2-Sulfanylpropanoyl)Phenylalanylalanine; N-(4-(2-((3-Chlorophenylmethyl)Amino)Ethyl)Phenyl)-2-Thiophecarboxamidine; N-(3-Propylcarbamoyloxirane-2-Carbonyl)-Isoleucyl-Proline; N-(4-CHLOROPHENYL)-1,2,3,4-TETRAHYDROISOQUINOLINE-7-SULFONAMIDE; N-(4-{[amino(imino)methyl]amino}butyl)-2,4′-bi-1,3-thiazole-4-carboxamide; N-(4-Aminobutanoyl)-S-(4-Methoxybenzyl)-L-Cysteinylglycine; N-(4-carbamimidoylbenzyl)-1-(3-phenylpropanoyl)-L-prolinamide; N-(4-carbamimidoylbenzyl)-1-(4-methylpentanoyl)-L-prolinamide; N-(4-chlorobenzyl)-N-methylbenzene-1,4-disulfonamide; N(4)-adenosyl-N(4)-methyl-2,4-diaminobutanoic acid; N-(4-CHLOROPHENYL)-3-(PHOSPHONOOXY)NAPHTHALENE-2-CARBOXAMIDE; N-(4-chlorophenyl)-2-[(pyridin-4-ylmethyl)amino]benzamide; N-(4-Methoxybenzyl)-N′-(5-Nitro-1,3-Thiazol-2-Yl)Urea; N-(4-METHYLBENZOYL)-4-BENZYLPIPERIDINE; (2Z,4E)-2-HYDROXY-6-OXO-6-PHENYLHEXA-2,4-DIENOIC ACID; N-(M-Trifluoromethylphenyl) Phenoxazine-4,6-Dicarboxylic Acid; N-(PARA-GLUTARAMIDOPHENYL-ETHYL)-PIPERIDINIUM-N-OXIDE; N-(P-CYANOPHENYL)-N′-DIPHENYLMETHYL-GUANIDINE-ACETIC ACID; N-(cyclopropylmethyl)-2′-methyl-5′-(5-methyl-1,3,4-oxadiazol-2-yl)biphenyl-4-carboxamide; N′-(6-aminopyridin-3-yl)-N-(2-cyclopentylethyl)-4-methyl-benzene-1,3-dicarboxamide; N-(6,7,9,10,17,18,20,21-octahydrodibenzo[b,k][1,4,7,10,13,16]hexaoxacyclooctadecin-2-yl)acetamide; N-(6-{[3-(4-Bromophenyl)-1,2-Benzisothiazol-6-Yl]Oxy}Hexyl)-N-Methylprop-2-En-1-Amine; N-[(2S,4S,6R)-2-(dihydroxymethyl)-4-hydroxy-3,3-dimethyl-7-oxo-4lambda˜4μ-thia-1-azabicyclo[3.2.0]hept-6-yl]-2-phenylacetamide; N-(4-OXO-5,6,7,8-TETRAHYDRO-4H-[1,3]THIAZOLO[5,4-C]AZEPIN-2-YL)ACETAMIDE; N-(4-phenoxyphenyl)-2-[(pyridin-4-ylmethyl)amino]nicotinamide; N-(4-PHENYLAMINO-QUINAZOLIN-6-YL)-ACRYLAMIDE; N-(4-sulfamoylphenyl)-1H-indazole-3-carboxamide; (2Z)-N-biphenyl-4-yl-2-cyano-3-hydroxybut-2-enamide; (3e)-3-[(4-Hydroxyphenyl)Imino]-1h-Indol-2(3h)-One; N-(5,5,8,8-Tetramethyl-5,8-Dihydro-Naphthalen-2-Yl)-Terephthalamic Acid; N-(5,6-DIPHENYLFURO[2,3-D]PYRIMIDIN-4-YL)GLYCINE; N-(5-{3,4-difluoro-2-[(2-fluoro-4-iodophenyl)amino]phenyl}-1,3,4-oxadiazol-2-yl)ethane-1,2-diamine; N′-(5-chloro-1,3-benzodioxol-4-yl)-N-(3-methylsulfonylphenyl)pyrimidine-2,4-diamine; (3E)-4-(2-HYDROXYPHENYL)-2-OXOBUT-3-ENOIC ACID; N-(5-chloro-1,3-benzodioxol-4-yl)-6-methoxy-7-(3-piperidin-1-ylpropoxy)quinazolin-4-amine; N-(5-Cyclopropyl-1h-Pyrazol-3-Yl)Benzamide; N-(5-ISOPROPYL-THIAZOL-2-YL)-2-PYRIDIN-3-YL-ACETAMIDE; N-(5-METHYL-1H-PYRAZOL-3-YL)-2-PHENYLQUINAZOLIN-4-AMINE; N-(6-Aminohexyl)-5-Chloro-1-Naphthalenesulfonamide; (3R)-3-cyclopentyl-7-[(4-methylpiperazin-1-yl)sulfonyl]-3,4-dihydro-2H-1,2-benzothiazine 1,1-dioxide; N-(biphenyl-4-ylsulfonyl)-D-leucine; N-(carboxycarbonyl)-D-phenylalanine; (3r)-4-(P-Toluenesulfonyl)-1,4-Thiazane-3-Carboxylicacid-L-Phenylalanine Ethyl Ester; N-(dibenzo[b,d]thiophen-3-ylsulfonyl)-L-valine; (3R)-3-hydroxy-2,2-dimethyl-4-oxo-4-({3-oxo-3-[(2-sulfanylethyl)amino]propyl}amino)butyl 2,2-dimethylpropanoate; (3R)-4-[(3R)-3-AMINO-4-(2,4,5-TRIFLUOROPHENYL)BUTANOYL]-3-METHYL-1,4-DIAZEPAN-2-ONE; N-(pyridin-3-ylmethyl)aniline; N′-(Pyrrolidino[2,1-B]Isoindolin-4-On-8-Yl)-N-(Pyridin-2-Yl)Urea; (3R)—N-(4-CHLOROPHENYL)-3-(HYDROXYMETHYL)-1,2,3,4-TETRAHYDROISOQUINOLINE-7-SULFONAMIDE; N-(R-Carboxy-Ethyl)-Alpha-(S)-(2-Phenylethyl); N-(R-Carboxy-Ethyl)-Alpha-(S)-(2-Phenylethyl)Glycyl-L-Arginine-N-Phenylamide; (3R,4S)-1-[(4-AMINO-5H-PYRROLO[3,2-D]PYRIMIDIN-7-YL)METHYL]-4-[(METHYLSULFANYL)METHYL]PYRROLIDIN-3-OL; N-(tert-butyl)-4-[5-(pyridin-2-ylamino)quinolin-3-yl]benzenesulfonamide; N-(TRANS-4′-NITRO-4-STILBENYL)-N-METHYL-5-AMINO-PENTANOIC ACID; N,4-Dihydroxy-N-Oxo-3-(Sulfooxy)Benzenaminium; N,4-dimethyl-3-[(1-phenyl-1H-pyrazolo[3,4-d]pyrimidin-4-yl)amino]benzamide; N,N′-[biphenyl-4,4′-diyldi(2R)propane-2,1-diyl]dimethanesulfonamide; N,N-bis(3-(D-gluconamido)propyl)deoxycholamide; N,N′-Bis(4-Amino-2-Methylquinolin-6-Yl)Urea; (3R,4S)-1-{6-[3-(METHYLSULFONYL)PHENYL]PYRIMIDIN-4-YL}-4-(2,4,5-TRIFLUOROPHENYL)PYRROLIDIN-3-AMINE; N-[1-(5-bromo-2,3-dimethoxybenzyl)piperidin-4-yl]-4-sulfanylbutanamide; N-[1-(4-Bromophenyl)Ethyl]-5-Fluoro Salicylamide; (3R,4S,5S,7R,9E,11R,12R)-12-ETHYL-4-HYDROXY-3,5,7,11-TETRAMETHYLOXACYCLODODEC-9-ENE-2,8-DIONE; (3R,5Z,8S,9S,11E)-8,9,16-TRIHYDROXY-14-METHOXY-3-METHYL-3,4,9,10-TETRAHYDRO-1H-2-BENZOXACYCLOTETRADECINE-1,7(8H)-DIONE; N-[1H-INDOL-3-YL-ACETYL]GLYCINE ACID; N-[3-(1H-BENZIMIDAZOL-2-YL)-1H-PYRAZOL-4-YL]BENZAMIDE; N-[2-chloro-5-(trifluoromethyl)phenyl]imidodicarbonimidic diamide; N-[2-(1h-Indol-5-Yl)-Butyl]-4-Sulfamoyl-Benzamide; N-[2-(1-Maleimidyl)Ethyl]-7-Diethylaminocoumarin-3-Carboxamide; N-[2-(METHYLAMINO)ETHYL]-5-ISOQUINOLINESULFONAMIDE; N-[2-(2-methyl-1H-Indol-3-yl)ethyl]thiophene-2-carboxamide; N-[2-(2-Iodo-5-methoxy-1H-Indol-3-yl)ethyl]acetamide; N-[1H-INDOL-3-YL-ACETYL]VALINE ACID; (3S)-1-CYCLOHEXYL-N-(3,5-DICHLOROPHENYL)-5-OXOPYRROLIDINE-3-CARBOXAMIDE; N-[4-(2-Methylimidazo[1,2-a]Pyridin-3-Yl)-2-Pyrimidinyl]Acetamide; (3S)—N-(3-CHLORO-2-METHYLPHENYL)-1-CYCLOHEXYL-5-OXOPYRROLIDINE-3-CARBOXAMIDE; N-[4-(5-fluoro-6-methylpyridin-2-yl)-5-quinoxalin-6-yl-1H-Imidazol-2-yl]acetamide; N-[4-(AMINOSULFONYL)PHENYL]-2-MERCAPTOBENZAMIDE; N-[4-CHLORO-3-(PYRIDIN-3-YLOXYMETHYL)-PHENYL]-3-FLUORO-; N-[4-CLORO-3-(T-BUTYLOXOME)PHENYL-2-METHYL-3-FURAN-CARBOTHIAMIDE; (3S,5R,7R,8S,9S,10R)-7-(hydroxymethyl)-3-(4-methylphenyl)-1,6-dioxa-2-azaspiro[4.5]decane-8,9,10-triol; N-[4-Methyl-3-[[4-(3-Pyridinyl)-2-Pyrimidinyl]Amino]Phenyl]-3-Pyridinecarboxamide; N-[amino(imino)methyl]-2-(2,5-diphenyl-1H-pyrrol-1-yl)acetamide; N-[amino(imino)methyl]-2-[2-(2-chlorophenyl)-4-(4-propoxyphenyl)-3-thienyl]acetamide; N-[Isoleucinyl]-N′-[Adenosyl]-Diaminosufone; (4R)—N-[4-({[2-(DIMETHYLAMINO)ETHYL]AMINO}CARBONYL)-1,3-THIAZOL-2-YL]-4-METHYL-1-OXO-2,3,4,9-TETRAHYDRO-1H-BETA-CARBOLINE-6-CARBOXAMIDE; (3s,8ar)-3-(4-Hydroxybenzyl)Hexahydropyrrolo[1,2-a]Pyrazine-1,4-Dione; N-[Tosyl-D-Prolinyl]Amino-Ethanethiol; N-{[4-(but-2-yn-1-yloxy)phenyl]sulfonyl}-5-methyl-D-tryptophan; N˜3˜-(3-PYRIDIN-3-YLBENZYL)PYRIDINE-2,3-DIAMINE; N˜3˜-[3-(1H-INDOL-6-YL)BENZYL]PYRIDINE-2,3-DIAMINE; (3Z,5S,6R,7S,8R,8aR)-3-(octylimino)hexahydro[1,3]oxazolo[3,4-a]pyridine-5,6,7,8-tetrol; N-butyl-3-{[6-(9H-purin-6-ylamino)hexanoyl]amino}benzamide; (3Z,5S,6R,7S,8S,8aR)-3-(octylimino)hexahydro[1,3]oxazolo[3,4-a]pyridine-5,6,7,8-tetrol; N-cyclopropyl-2′,6-dimethyl-4′-(5-methyl-1,3,4-oxadiazol-2-yl)biphenyl-3-carboxamide; N-cyclohexyl-4-imidazo[1,2-a]pyridin-3-yl-N-methylpyrimidin-2-amine; N-cyclooctylglycyl-N-(4-carbamimidoylbenzyl)-L-prolinamide; N-cyclohexyl-3-[3-(trifluoromethyl)phenyl][1,2,4]triazolo[4,3-b]pyridazin-6-amine; N-cyclopropyl-4-methyl-3-[1-(2-methylphenyl)phthalazin-6-yl]benzamide; N-cyclopropyl-4-methyl-3-{2-[(2-morpholin-4-ylethyl)amino]quinazolin-6-yl}benzamide; (4aS,4bR,10bS,12aS)-12a-methyl-1,3-dioxo-2-(pyridin-3-ylmethyl)-1,2,3,4,4a,4b,5,6,10b,11,12,12a-dodecahydronaphtho[2,1-f]isoquinolin-8-ylsulfamate; N-cyclopropyl-6-[(6,7-dimethoxyquinolin-4-yl)oxy]naphthalene-1-carboxamide; Ndelta-(N′-Sulphodiaminophosphinyl)-L-Ornithine; N-Dodecyl-N,N-Dimethyl-3-Ammonio-1-Propanesulfonate; (4R)-7,8-dichloro-1′,9-dimethyl-1-oxo-1,2,4,9-tetrahydrospiro[beta-carboline-3,4′-piperidine]-4-carbonitrile; Nicotinamide 8-Bromo-Adenine Dinucleotide Phosphate; (4R,2S)-5′-(4-(4-CHLOROBENZYLOXY)PYRROLIDIN-2-YLMETHANESULFONYL)ISOQUINOLINE; (4Z)-2,8:7,12:11,15:14,18:17,22-PENTAANHYDRO-4,5,6,9,10,13,19,20,21-NONADEOXY-D-ARABINO-D-ALLO-D-ALLO-DOCOSA-4,9,20-TRIENITOL; Nicotinamide Adenine Dinucleotide Cyclohexanone; Nicotinamide-Adenine-Dinucleotide-5-Hydroxy-4-Oxonorvaline; N-methyl-1-(2-thiophen-2-ylphenyl)methanamine; N-METHYL-1-[4-(9H-PURIN-6-YL)PHENYL]METHANAMINE; N-METHYL-N-[(1R)-1-METHYL-2-PHENYLETHYL]PROP-2-EN-1-AMINE; N-Methyl-N-[3-(6-Phenyl[1,2,4]Triazolo[4,3-B]Pyridazin-3-Yl)Phenyl]Acetamide; N-Methyl-N-Propargyl-3-(2,4-Dichlorophenoxy)Propylamine; (5-{3-[5-(PIPERIDIN-1-YLMETHYL)-1H-INDOL-2-YL]-1H-INDAZOL-6-YL}-2H-1,2,3-TRIAZOL-4-YL)METHANOL; N-Octanoyl-B-D-Fructofuranosyl-a-D-Glucopyranoside, Sucrose Monocaproylate; N-oxo-2-[(4-phenylphenyl)sulfonylamino]ethanamide; (5-(aminomethyl)-2H-spiro[benzofuran-3,4′-piperidine]-1)(5-(phenylethynyl)furan-2-yl)methanone; N-PYRIDOXYL-2-METHYL-L-GLUTAMIC ACID-5′-MONOPHOSPHATE; N-Pyridoxyl-7-Keto-8-Aminopelargonic Acid-5′-Monophosphate; N-PYRIDOXYL-D-GLUTAMIC ACID-5′-MONOPHOSPHATE; 0-(2-Acetamido-2-Deoxy-Alpha-D-Galactopyranosyl)-L-Serine; 0,0-DIETHYL HYDROGEN THIOPHOSPHATE; (5E)-12-CHLORO-13,15-DIHYDROXY-4,7,8,9-TETRAHYDRO-2-BENZOXACYCLOTRIDECINE-1,10(3H,11H)-DIONE; ORTHONITROPHENYL-BETA-D-FUCOPYRANOSIDE; P-(2′-Iodo-5′-Thenoyl)Hydrotropic Acid; (5R)-5-(4-{[(2R)-6-HYDROXY-2,5,7,8-TETRAMETHYL-3,4-DIHYDRO-2H-CHROMEN-2-YL]METHOXY}BENZYL)-1,3-THIAZOLIDINE-2,4-DIONE; P1-(5-Adenosyl)P5-(5′-Thymidyl)Pentaphosphate; P1-(Adenosine-5′-P5-(Uridine-5′)Pentaphosphate; PA mAb (human monoclonal antibody to Bacillus anthracis protective antigen); PANTOTHENYL-AMINOETHANOL-11-PIVALIC ACID; Pantothenyl-Aminoethanol-Acetate Pivalic Acid; O-[2-(1,3-dioxo-1,3-dihydro-2H-isoindol-2-yl)ethyl] (4-bromophenyl)thiocarbamate; O-[2-(1,3-dioxo-1,3-dihydro-2H-isoindol-2-yl)ethyl] (4-chlorophenyl)thiocarbamate; O-[2-(1,3-dioxo-1,3-dihydro-2H-isoindol-2-yl)ethyl] (4-iodophenyl)thiocarbamate; O6-CYCLOHEXYLMETHOXY-2-(4′-SULPHAMOYLANILINO) PURINE; (5R)-4-HYDROXY-3,5-DIMETHYL-5-[(1E,3E)-2-METHYLPENTA-1,3-DIENYL]THIOPHEN-2(5H)-ONE; PARA-NITROPHENYL 1-THIO-BETA-D-GLUCOPYRANOSIDE; PARA-NITROPHENYL PHOSPHONOBUTANOYL D-ALANINE; PARA-NITROPHENYL PHOSPHONOBUTANOYL L-ALANINE; PARA-NITROPHENYLPHOSPHONOBUTANOYL-GLYCINE; PHENYL[1-(N-SUCCINYLAMINO)PENTYL]PHOSPHONATE; PHENYL-5-(1H-PYRAZOL-3-YL)-1,3-THIAZOLE; (5r)-5-Amino-6-Hydroxyhexylcarbamic Acid; Phosphoaminophosphonic Acid-Adenylate Ester; (5r,6s,7s,8s)-5-Hydroxymethyl-6,7,8-Trihydroxy-Tetrazolo[1,5-a]Piperidine; Phosphoric Acid-2′-[2′-Deoxy-Uridine]Ester-5′-Guanosine Ester; (5R)-N,N-DIETHYL-5-METHYL-2-[(THIOPHEN-2-YLCARBONYL)AMINO]-4,5,6,7-TETRAHYDRO-1-BENZOTHIOPHENE-3-CARBOXAMIDE; (5S)-2-{[(1S)-1-(2-fluorophenyl)ethyl]amino}-5-methyl-5-(trifluoromethyl)-1,3-thiazol-4(5H)-one; Phosphomethylphosphonic Acid Guanosyl Ester; (5S)-5-(2-amino-2-oxoethyl)-4-oxo-N-[(3-oxo-3,4-dihydro-2H-1,4-benzoxazin-6-yl)methyl]-3,4,5,6,7,8-hexahydro[1]benzothieno[2,3-d]pyrimidine-2-carboxamide; PIPERIDINE-2-CARBOXYLIC ACID TERT-BUTYLAMIDE; Pmp-Hydroxyisoxazole, Pyridoxamine-5-Phosphate-Hydroxyisoxazole; PROGESTERONE-11-ALPHA-OL-HEMISUCCINATE; (5Z)-12-CHLORO-13,15-DIHYDROXY-4,7,8,9-TETRAHYDRO-2-BENZOXACYCLOTRIDECINE-1,10(3H,11H)-DIONE; rGLP-1 (continuous infusion of glucagon-like peptide 1); rhGAD65 (recombinant human glutamic acid decarboxylase); (5Z)-13-CHLORO-14,16-DIHYDROXY-3,4,7,8,9,10-HEXAHYDRO-1H-2-BENZOXACYCLOTETRADECINE-1,11(12H)-DIONE; S-(N-Hydroxy-N-Bromophenylcarbamoyl)Glutathione; S-(N-Hydroxy-N-Iodophenylcarbamoyl)Glutathione; S,S′-(1,3-Phenylene-Bis(1,2-Ethanediyl))Bis-Isothiourea; S,S′-(1,4-Phenylene-Bis(1,2-Ethanediyl))Bis-Isothiourea; Selenazole-4-Carboxyamide-Adenine Dinucleotide; (5Z)-5-[(5-ETHYL-2-FURYL)METHYLENE]-2-{[(S)-(4-FLUOROPHENYL)(1H-TETRAZOL-5-YL)METHYL]AMINO}-1,3-THIAZOL-4(5H)-ONE; S-METHYL-4,5,6,7-TETRABROMO-BENZIMIDAZOLE; Sodium phosphate, dibasic, heptahydrate; (6E)-7-{6-[(1E)-OCT-1-ENYL]-2,3-DIAZABICYCLO[2.2.1]HEPT-2-EN-5-YL}HEPT-6-ENOIC ACID; (6AR,11AS,11BR)-10-ACETYL-9-HYDROXY-7,7-DIMETHYL-2,6,6A,7,11A,11B-HEXAHYDRO-11H-PYRROLO[1′,2′:2,3]ISOINDOLO[4,5,6-CD]INDOL-11-ONE; Sp-Adenosine-3′,5′-Cyclic-Monophosphorothioate; Sulfoquinovose-Uridine-C1,5′-Diphosphate; Tazobactam Trans-Enamine Intermediate; (6-METHYL-3,4-DIHYDRO-2H-CHROMEN-2-YL)METHYLPHOSPHINATE; Tert-Butyl(1s)-1-Cyclohexyl-2-Oxoethylcarbamate; TETRAHEDRAL INTERMEDIATE OF BLASTICIDIN S; trans-4-(7-carbamoyl-1H-benzimidazol-2-yl)-1-propylpiperidinium; Trans-6-(2-Phenylcyclopropyl)-Naphthalene-2-Carboxamidine; tripotassium (1R)-4-biphenyl-4-yl-1-phosphonatobutane-1-sulfonate; (6r,7r)-3-[(Acetyloxy)Methyl]-7-{[(6s)-6-(Glycylamino)-7-Oxido-7-Oxoheptanoyl]Amino}-8-Oxo-5-Thia-1-Azabicyclo[4.2.0]Octane-2-Carboxylate; Uridine-5′-Diphosphate-2-Deoxy-2-Fluoro-Alpha-D-Galactose; Uridine-5′-Diphosphate-4-Deoxy-4-Fluoro-Alpha-D-Galactose; (8E,10S,12Z)-10-hydroxy-6-oxooctadeca-8,12-dienoic acid; 4-[[2-[[4-chloro-3-(trifluoromethyl)phenyl]amino]-3H-benzimidazol-5-yl]oxy]-N-methyl-pyridine-2-carboxamide; Vitamin B6 Complexed with 2-Amino-Hexanoic Acid; Vitamin B6 Complexed with 2-Amino-Pentanoic Acid; Uridine-5′-Diphosphate-N-Acetylmuramoyl-L-Alanine-D-Glutamate; 4-[1-(2,6-dichlorobenzyl)-2-methyl-1H-imidazol-4-yl]pyrimidin-2-amine; 4-[5-(2-CARBOXY-1-FORMYL-ETHYLCARBAMOYL)-PYRIDIN-3-YL]-BENZOIC ACID; 4-[4-AMINO-6-(5-CHLORO-1H-INDOL-4-YLMETHYL)-[1,3,5]TRIAZIN-2-YLAMINO]-BENZONITRILE; 4-[4-AMINO-6-(2,6-DICHLORO-PHENOXY)-[1,3,5]TRIAZIN-2-YLAMINO]-BENZONITRILE; 4-[5-(3-IODO-PHENYL)-2-(4-METHANESULFINYL-PHENYL)-1H-IMIDAZOL-4-YL]-PYRIDINE; (9Z, 11E, 13S)-13-hydroxyoctadeca-9,11-dienoic acid; 4-Bromo-3-(5-Carboxy-4′-Chloro-2′-Fluorophenyl)-1-Methyl-5-Trifluoromethyl-Pyrazol; 4-[5-[2-(1-Phenyl-Ethylamino)-Pyrimidin-4-Yl]-1-Methyl-4-(3-Trifluoromethylphenyl)-1h-Imidazol-2-Yl]-Piperidine; 4-BROMO-2-FLUORO-N-[(4E)-6-METHOXY-7-[(1-METHYLPIPERIDIN-4-YL)METHOXY]QUINAZOLIN-4(1H)-YLIDEN]ANILINE; 4-[5-(Trans-4-Aminocyclohexylamino)-3-Isopropylpyrazolo[1,5-a]Pyrimidin-7-Ylamino]-N,N-Dimethylbenzenesulfonamide; 4-BROMO-3-(CARBOXYMETHOXY)-5-(4-HYDROXYPHENYL)THIOPHENE-2-CARBOXYLIC ACID; (E)-(45,65)-8-METHYL-6-((S)-3-METHYL-2-{(S)-2-[(5-METHYL-ISOXAZOLE-3-CARBONYL)-AMINO]-PROPIONYLAMINO}-BUTYRYLAMINO)-5-OXO-4-((R)-2-OXO-PYRROLIDIN-3-YLMETHYL)-NON-2-ENOIC ACID BENZYL ESTER; 4-{(Z)-[2-[3-(Methylsulfanyl)Propanoyl]-5-Oxo-1-(2-Oxoethyl)-1,5-Dihydro-4h-Imidazol-4-Ylidene]Methyl}Benzenolate; (E)-(S)-4-[(S)-4-METHYL-2-((S)-3-METHYL-2{(S)-2-[(5-METHYL-ISOXAZOLE-3-CARBONYL)-AMINO]-PROPIONYLAMINO}-BUTYRYLAMINO)-PENTANOYLAMINO]-5-((S)-2-OXO-PYRROLIDIN-3-YL)-PENT-2-ENOIC ACID ETHYL ESTER; 4-{(1E)-3-OXO-3-[(2-PHENYLETHYL)AMINO]PROP-1-EN-1-YL}-1,2-PHENYLENE DIACETATE; 4-BROMO-3-(CARBOXYMETHOXY)-5-PHENYLTHIOPHENE-2-CARBOXYLIC ACID; 4-{[(14beta,17alpha)-3-hydroxyestra-1,3,5(10)-trien-17-yl]oxy}-4-oxobutanoic acid; 4-{4-[2-(1A,7A-DIMETHYL-4-OXY-OCTAHYDRO-1-OXA-4-AZA-CYCLOPROPA[A]NAPHTHALEN-4-YL)-ACETYLAMINO]-PHENYLCARBAMOYL}-BUTYRIC ACID; 4-{4-[3-(2,4-DICHLORO-BENZOYL)-UREIDO]-2,3-DIMETHYL-PHENOXY}-BUTYRIC ACID; 4-{4-[4-(3-AMINOPROPOXY)PHENYL]-1H-PYRAZOL-5-YL}-6-CHLOROBENZENE-1,3-DIOL; (R)-1-(4-(4-(HYDROXYMETHYL)-1,3,2-DIOXABOROLAN-2-YL)PHENYL)GUANIDINE; 4-{5-[(Z)-(2,4-DIOXO-1,3-THIAZOLIDIN-5-YLIDENE)METHYL]FURAN-2-YL}BENZENESULFONAMIDE; 4-{5-[(Z)-(2-IMINO-4-OXO-1,3-THIAZOLIDIN-5-YLIDENE)METHYL]-2-FURYL}-N-METHYLBENZENESULFONAMIDE; 4-{5-[(Z)-(2-IMINO-4-OXO-1,3-THIAZOLIDIN-5-YLIDENE)METHYL]FURAN-2-YL}-2-(TRIFLUOROMETHYL)BENZENESULFONAMIDE; (E)-3,4-DIHYDROXY-N′-[(2-METHOXYNAPHTHALEN-1-YL)METHYLENE]BENZOHYDRAZIDE; 4-{5-[(Z)-(2-IMINO-4-OXO-1,3-THIAZOLIDIN-5-YLIDENE)METHYL]FURAN-2-YL}BENZENESULFONAMIDE; 4-{5-[(Z)-(2-IMINO-4-OXO-1,3-THIAZOLIDIN-5-YLIDENE)METHYL]FURAN-2-YL}BENZOIC ACID; (R)-1-(4-(4-(HYDROXYMETHYL)-1,3,2-DIOXABOROLAN-2-YL)BENZYL)GUANIDINE; (E)-3-(5((5-(4-CHLOROPHENYL)FURAN-2-YL)METHYLENE)-4-OXO-2-THIOXOTHIAZOLIDIN-3-YL)PROPANOIC ACID; 4-{[(2,6-difluorophenyl)carbonyl]amino}-N-[(3S)-piperidin-3-yl]-1H-pyrazole-3-carboxamide; (S)-1-PHENYL-1-[4-(9H-PURIN-6-YL)PHENYL]METHANAMINE; 4-(1R,3AS,4R,8AS,8BR)-[1-DIFLUOROMETHYL-2-(4-FLUOROBENZYL)-3-OXODECAHYDROPYRROLO[3,4-A]PYRROLIZIN-4-YL]BENZAMIDINE; 4-(2-AMINOETHOXY)-3,5-DICHLORO-N-[3-(1-METHYLETHOXY)PHENYL]BENZAMIDE; 4-(2-chlorophenyl)-8-(2-hydroxyethyl)-6-methylpyrrolo[3,4-e]indole-1,3(2H,6H)-dione; 4-Amino-N-{4-[2-(2,6-Dimethyl-Phenoxy)-Acetylamino]-3-Hydroxy-1-Isobutyl-5-Phenyl-Pentyl}-Benzamide; (R)┌ÇöN[2-[1-(aminoiminomethyl)-3-piperidinyl]-1-oxoethyl]-4-(phenylethynyl)-1-phenylalanine methylester; 4-(2-HYDROXY-4-FLUOROPHENYLTHIO)-BUTYLPHOSPHONIC ACID; 4-(5,11-DIOXO-5H-INDENO[1,2-C]ISOQUINOLIN-6(11H)-YL)BUTANOATE; 4-[(5-CHLOROINDOL-2-YL)SULFONYL]-2-(2-METHYLPROPYL)-1-[[5-(PYRIDIN-4-YL)PYRIMIDIN-2-YL]CARBONYL]PIPERAZINE; (S)-2-[(R)-3-amino-4-(2-fluorophenyl)butyryl]-1,2,3,4-tetrahydroisoquinoline-3-carboxamide; 4-(5-BENZENESULFONYLAMINO-1-METHYL-1H-BENZOIMIDAZOL-2-YLMETHYL)-BENZAMIDINE; 4-[2-(3-Benzyloxycarbonylamino-4-Cyclohexyl-1-Hydroxy-2-Oxo-Butylamino)-5-Guanidino-Pentanoylamino]-4-(1-Carboxy-2-Cyclohexyl-Ethylcarbamoyl)-Butyric Acid; 4-(4-CHLORO-PHENYL)-1-{3-[2-(4-FLUORO-PHENYL)-[1,3]DITHIOLAN-2-YL]-PROPYL}-PIPERIDIN-4-OL; 4-(4-CHLOROPHENYL)-4-[4-(1H-PYRAZOL-4-YL)PHENYL]PIPERIDINE; 4-[3-Oxo-3-(5,5,8,8-Tetramethyl-5,6,7,8-Tetrahydro-Naphthalen-2-Yl)-Propenyl]-Benzoic Acid; 4-[4-(1-Amino-1-Methylethyl)Phenyl]-5-Chloro-N-[4-(2-Morpholin-4-Ylethyl)Phenyl]Pyrimidin-2-Amine; (S)-2-Amino-3-(4h-Selenolo[3,2-B]-Pyrrol-6-Yl)-Propionic Acid; 4-[4-(2,4,6-TRIMETHYL-PHENYLAMINO)-PYRIMIDIN-2-YLAMINO]-BENZONITRILE; [(2-Ethoxy-1-Naphthoyl)Amino]Methylboronic Acid; 4-[4-(4-CHLORO-PHENOXY)-BENZENESULFONYLMETHYL]-TETRAHYDRO-PYRAN-4-CARBOXYLIC ACID HYDROXYAMIDE; 4-HYDROXY-3-[(1S,3R)-3-HYDROXY-1-PHENYLBUTYL]-2H-CHROMEN-2-ONE; 4-HYDROXY-3-[(1S,3S)-3-HYDROXY-1-PHENYLBUTYL]-2H-CHROMEN-2-ONE; 4-PHENOXY-N-(PYRIDIN-2-YLMETHYL)BENZAMIDE; 4-PHOSPHONOOXY-PHENYL-METHYL-[4-PHOSPHONOOXY]BENZEN; 4-CHLORO-6-(4-{4-[4-(METHYLSULFONYL)BENZYL]PIPERAZIN-1-YL}-1H-PYRAZOL-5-YL)BENZENE-1,3-DIOL; 4-CHLORO-6-(4-PIPERAZIN-1-YL-1H-PYRAZOL-5-YL)BENZENE-1,3-DIOL; 4-HYDROXY-3-NITROPHENYLACETYL-EPSILON-AMINOCAPROIC ACID ANION; 4-HYDROXY-5-IODO-3-NITROPHENYLACETYL-EPSILON-AMINOCAPROIC ACID ANION; 4-HYDROXY-7-METHOXY-3-(1-PHENYL-PROPYL)-CHROMEN-2-ONE; (S)—N-(1-(3-CHLORO-4-FLUOROPHENYL)-2-HYDROXYETHYL)-4-(4-(3-CHLOROPHENYL)-1H-PYRAZOL-3-YL)-1H-PYRROLE-2-CARBOXAMIDE; 4-METHYL-5-{(2E)-2-[(4-MORPHOLIN-4-YLPHENYL)IMINO]-2,5-DIHYDROPYRIMIDIN-4-YL}-1,3-THIAZOL-2-AMINE; 4-NITROPHENYL-(6-S-ALPHA-D-XYLOPYRANOSYL)-BETA-D-GLUCOPYRANOSIDE; 4-CHLORO-8-METHYL-7-(3-METHYL-BUT-2-ENYL)-6,7,8,9-TETRAHYDRO-2H-2,7,9A-TRIAZA-BENZO[CD]AZULENE-1-THIONE; [(1S)-1-(5-CHLORO-1-BENZOTHIEN-3-YL)-2-(2-NAPHTHYLAMINO)-2-OXOETHYL]PHOSPHONIC ACID; 4-tert-butyl-N′-[(1E)-(3,5-dibromo-2,4-dihydroxyphenyl)methylidene]benzohydrazide; 5-(2-ETHOXYETHYL)-5-[4-(4-FLUOROPHENOXY)PHENOXY]PYRIMIDINE-2,4,6(1H,3H,5H)-TRIONE; 4-chloro-N-[(2S)-2-methyl-2,3-dihydro-1H-indol-1-yl]-3-sulfamoylbenzamide; 4-chloro-N′-[(1E)-(3,5-dibromo-2,4-dihydroxyphenyl)methylidene]benzohydrazide; 5-(2-fluorophenyl)-N-(pyridin-4-ylmethyl)pyrazolo[1,5-a]pyrimidin-7-amine; 4-Dimethylamino-N-(6-Hydroxycarbamoyethyl)Benzamide-N-Hydroxy-7-(4-Dimethyla Minobenzoyl)Aminoheptanamide; 5-(2-PHENYLPYRAZOLO[1,5-A]PYRIDIN-3-YL)-1H-PYRAZOLO[3,4-C]PYRIDAZIN-3-AMINE; 5-(3-(2,6-dichloro-4-(4,5-dihydro-2-oxazolyl)phenoxy)propyl)-3-methyl isoxazole; 4′-Deaza-1′-Aza-2′-Deoxy-1′-(9-Methylene)-Immucillin-H, (3r,4r)-N-[9-Deazahypoxanthin-9-Yl)Methyl]-4-Hydroxymethyl-Pyrrolidin-3-Ol; 4-Deoxy-4-((5-Hydroxymethyl-2,3,4-Trihydroxycyclohex-5,6-Enyl)Amino)Fructose; 4-bromo-N′-[(1E)-(3,5-dibromo-2,4-dihydroxyphenyl)methylidene]benzohydrazide; 4-Carbamoyl-1-Beta-D-Ribofuranosyl-Imidazolium-5-Olate-5′-Phosphate; [(4S)-2,2-DIMETHYL-1,3-DIOXOLAN-4-YL]METHYL HYDROGEN HEX-5-ENYLPHOSPHONATE; 4-AMINO-N-[(2-SULFANYLETHYL)CARBAMOYL]BENZENESULFONAMIDE; 4-{[5-chloro-4-(1H-indol-3-yl)pyrimidin-2-yl]amino}-N-ethylpiperidine-1-carboxamide; 5-(2,6-dichlorophenyl)-2-[(2,4-difluorophenyl)sulfanyl]-6H-pyrimido[1,6-b]pyridazin-6-one; 5-(5-(6-CHLORO-4-(4,5-DIHYDRO-2-OXAZOLYL)PHENOXY)PENTYL)-3-METHYLISOXAZOLE; 5-(5-CHLORO-2,4-DIHYDROXYPHENYL)-N-ETHYL-4-(4-METHOXYPHENYL)-1H-PYRAZOLE-3-CARBOXAMIDE; 5-(5-CHLORO-2,4-DIHYDROXYPHENYL)-N-ETHYL-4-(4-METHOXYPHENYL)ISOXAZOLE-3-CARBOXAMIDE; 5-(5-chloro-2,4-dihydroxyphenyl)-N-ethyl-4-[4-(morpholin-4-ylmethyl)phenyl]isoxazole-3-carboxamide; 5-(5-CHLORO-2,4-DIHYDROXYPHENYL)-N-ETHYL-4-PIPERAZIN-1-YL-1H-PYRAZOLE-3-CARBOXAMIDE; 5-(5-CHLORO-2-THIENYL)-N-{(3S)-1-[(1S)-1-METHYL-2-MORPHOLIN-4-YL-2-OXOETHYL]-2-OXOPYRROLIDIN-3-YL}-1H-1,2,4-TRIAZOLE-3-SULFONAMIDE; 5-(5-chloro-7H-pyrrolo[2,3-d]pyrimidin-4-yl)-4,5,6,7-tetrahydro-1H-imidazo[4,5-c]pyridine; 5-(3,5-DICHLOROPHENYL)THIO-4-ISOPROPYL-1-(PYRIDIN-4-YL-METHYL)-1H-IMIDAZOL-2-YL-METHYL CARBAMATE; 5-[3-(2-METHOXYPHENYL)-1H-PYRROLO[2,3-B]PYRIDIN-5-YL]-N,N-DIMETHYLPYRIDINE-3-CARBOXAMIDE; [1-(6-{6-[(1-methylethyl)amino]-1H-indazol-1-yl}pyrazin-2-yl)-1H-pyrrol-3-yl]acetic acid; [2-(1-Amino-2-Hydroxy-Propyl)-4-(4-Fluoro-1h-Indol-3-Ylmethyl)-5-Hydroxy-Imidazol-1-Yl]-Acetic Acid; 5-{[(4-AMINO-3-CHLORO-5-FLUOROPHENYL)SULFONYL]AMINO}-1,3,4-THIADIAZOLE-2-SULFONAMIDE; 5-AMINO-2-{4-[(4-AMINOPHENYL)SULFANYL]PHENYL}-1H-ISOINDOLE-1,3(2H)-DIONE; 5-[4-(1-Carboxymethyl-2-Oxo-Propylcarbamoyl)-Benzylsulfamoyl]-2-Hydroxy-Benzoic Acid; 5-[5,6-BIS(METHYLOXY)-1H-BENZIMIDAZOL-1-YL]-3-{[1-(2-CHLOROPHENYL)ETHYL]OXY}-2-THIOPHENECARBOXAMIDE; 5-{2-[1-(1-METHYL-PROPYL)-7A-METHYL-OCTAHYDRO-INDEN-4-YLIDENE]-ETHYLIDENE}-2-METHYLENE-CYCLOHEXANE-1,3-DIOL; 5-{3-[3-(2,4-DICHLORO-BENZOYL)-UREIDO]-2-METHYL-PHENOXY}-PENTANOIC ACID; 5-{4-[(3,5-DIFLUOROBENZYL)AMINO]PHENYL}-6-ETHYLPYRIMIDINE-2,4-DIAMINE; 5-ACETAMIDO-5,6-DIHYDRO-4-HYDROXY-6-ISOBUTOXY-4H-PYRAN-2-CARBOXYLIC ACID; 5′-ACETYL-4-{[(2,4-DIMETHYLPHENYL)SULFONYL]AMINO}-2,2′-BITHIOPHENE-5-CARBOXYLIC ACID; [2-Cytidylate-O′-Phosphonyloxyl]-Ethyl-Trimethyl-Ammonium; 5-[2,3-Dichloro-4-(5-{1-[2-(2-Guanidino-4-Methyl-Pentanoylamino)-Acetyl]-Piperidin-4-Yl}-1-Methyl-1h-Pyrazol-3-Yl)-Phenoxymethyl]-Furan-2-Carboxylic Acid; 5-(3-{3-[3-HYDROXY-2-(METHOXYCARBONYL)PHENOXY]PROPENYL}PHENYL)-4-(HYDROXYMETHYL)ISOXAZOLE-3-CARBOXYLIC ACID; [4-(1,3,2-Dioxaborolan-2-Yloxy)Methyl]Benzamidine; 5-Bromo-N-(2,3-Dihydroxypropoxy)-3,4-Difluoro-2-[(2-Fluoro-4-Iodophenyl)Amino]Benzamide; 5-bromo-N-(3-chloro-2-(4-(prop-2-ynyl)piperazin-1-yl)phenyl)furan-2-carboxamide; 5-Chloro-1h-Indole-2-Carboxylic Acid [1-(4-Fluorobenzyl)-2-(4-Hydroxypiperidin-1yl)-2-Oxoethyl]Amide; 5-Bromo-N[2-(Dimethylamino)Ethyl]-9-Aminoacridine-4-Carboxamide; [4-({4-[(5-cyclopropyl-1H-pyrazol-3-yl)amino]-6-(methylamino)pyrimidin-2-yl}amino)phenyl]acetonitrile; 5-Chloro-6-[(2-Iminopyrrolidin-1-Yl)Methyl]Pyrimidine-2,4(1h,3h)-Dione; 5-CHLORO-6-METHYL-N-(2-PHENYLETHYL)-2-PYRIDIN-2-YLPYRIMIDIN-4-AMINE; 5-chloro-7-[(1-methylethyl)amino]pyrazolo[1,5-a]pyrimidine-3-carbonitrile; 5-CHLORO-8-METHYL-7-(3-METHYL-BUT-2-ENYL)-6,7,8,9-TETRAHYDRO-2H-2,7,9A-TRIAZA-BENZO[CD]AZULENE-1-THIONE; 5-CHLORO-N-((1R,2S)-2-(4-(2-OXOPYRIDIN-1(2H)-YL)BENZAMIDO) CYCLOPENTYL)THIOPHENE-2-CARBOXAMIDE; [4-(5-naphthalen-2-yl-1H-pyrrolo[2,3-b]pyridin-3-yl)phenyl]acetic acid; 5-CHLORO-N-(2-(4-(2-OXOPYRIDIN-1(2H)-YL)BENZAMIDO)ETHYL)THIOPHENE-2-CARBOXAMIDE; 5-chloro-N-[(3R)-1-(2-{[2-fluoro-4-(2-oxopyridin-1(2H)-yl)phenyl]amino}-2-oxoethyl)pyrrolidin-3-yl]thiophene-2-carboxamide; [4-(4-Hydroxy-Benzyl)-2-(2-Hydroxy-1-Methyl-Ethyl)-5-Oxo-Imidazolidin-1-Yl]-Acetic Acid; 5-CHLORO-N-{(3S)-1-[(1S)-1-METHYL-2-MORPHOLIN-4-YL-2-OXOETHYL]-2-OXOPYRROLIDIN-3-YL}-1-BENZOTHIOPHENE-2-SULFONAMIDE; 5-chloro-N-{4-[(1R)-1,2-dihydroxyethyl]phenyl}-1H-indole-2-carboxamide; 5-Chloro-thiophene-2-carboxylic acid ((3S,4S)-1-{[2-fluoro-4-(2-oxo-2H-pyridin-1-yl)-phenylcarbamoyl]-methyl}-4-hydroxy-pyrrolidin-3-yl)-amide; 5-CHLORO-THIOPHENE-2-CARBOXYLIC ACID ((3S,4S)-4-FLUORO-1-{[2-FLUORO-4-(2-OXO-2H-PYRIDIN-1-YL)-PHENYLCARBAMOYL]-METHYL)-PYRROLIDIN-3-YL)-AMIDE; 5-CYANO-FURAN-2-CARBOXYLIC ACID [5-HYDROXYMETHYL-2-(4-METHYL-PIPERIDIN-1-YL)-PHENYL]-AMIDE; 5-CYANO-N-(2,5-DIMETHOXYBENZYL)-6-ETHOXYPYRIDINE-2-CARBOXAMIDE; 5-cyclopropyl-2-(4-fluorophenyl)-6-[(2-hydroxyethyl)(methylsulfonyl)amino]-N-methyl-1-benzofuran-3-carboxamide; 5-ethoxy-4-(1-methyl-7-oxo-3-propyl-6,7-dihydro-1H-pyrazolo[4,3-d]pyrimidin-5-yl)thiophene-2-sulfonamide; 5-ETHYL-3-[(2-METHOXYETHYL)METHYLAMINO]-6-METHYL-4-(3-METHYLBENZYL)PYRIDIN-2(1H)-ONE; 5-ETHYL-3-METHYL-1,5-DIHYDRO-4H-PYRAZOLO[4,3-C]QUINOLIN-4-ONE; [5-(5-nitro-2-furyl)-1,3,4-oxadiazol-2-yl]thio}acetic acid; 5-(4-CYANOPHENYL)-3-{[(2-METHYLPHENYL)SULFONYL]AMINO}THIOPHENE-2-CARBOXYLIC ACID; 5-(4-CHLORO-5-PHENYL-3-THIENYL)-1,2,5-THIADIAZOLIDIN-3-ONE 1,1-DIOXIDE; 5-(4-FLUOROPHENYL)-3-{[(4-METHYLPHENYL)SULFONYL]AMINO}THIOPHENE-2-CARBOXYLIC ACID; 5-(4-METHOXYBIPHENYL-3-YL)-1,2,5-THIADIAZOLIDIN-3-ONE 1,1-DIOXIDE; [N-(2,4-DIAMINOPTERIDIN-6-YL)-METHYL]-DIBENZ[B,F]AZEPINE; 5-(5-(2,6-DICHLORO-4-(4,5-DIHYDRO-2-OXAZOLY)PHENOXY)PENTYL)-3-METHYLISOXAZOLE; [PHENYLALANINYL-PROLINYL]-[2-(PYRIDIN-4-YLAMINO)-ETHYL]-AMINE; 5-[1-(3,4-Dimethoxy-Benzoyl)-1,2,3,4-Tetrahydro-Quinolin-6-Yl]-6-Methyl-3,6-Dihydro-[1,3,4]Thiadiazin-2-One; 5-[1-(4-methoxyphenyl)-1H-benzimidazol-6-yl]-1,3,4-oxadiazole-2(3H)-thione; 5-[1-(Acetylamino)-3-Methylbutyl]-2,5-Anhydro-3,4-Dideoxy-4-(Methoxycarbonyl)Pentonic Acid; 5-[(E)-(5-CHLORO-2-OXO-1,2-DIHYDRO-3H-INDOL-3-YLIDENE)METHYL]-N-[2-(DIETHYLAMINO)ETHYL]-2,4-DIMETHYL-1H-PYRROLE-3-CARBOXAMIDE; 5-[(Z)-(5-CHLORO-2-OXO-1,2-DIHYDRO-3H-INDOL-3-YLIDENE)METHYL]-N-(DIETHYLAMINO)ETHYL]-2,4-DIMETHYL-1H-PYRROLE-3-CARBOXAMIDE; 5-[[(2R)-2-cyclopropyl-7,8-dimethoxy-2H-chromen-5-yl]methyl]pyrimidine-2,4-diamine; 5-[[(2R)-2-cyclopropyl-7,8-dimethoxy-chroman-5-yl]methyl]pyrimidine-2,4-diamine; 5-[(2-AMINOETHYL)AMINO]-6-FLUORO-3-(1H-PYRROL-2-YL)BENZO[CD]INDOL-2(1H)-ONE; 5-[(2-methyl-5-{[3-(trifluoromethyl)phenyl]carbamoyl}phenyl)amino]pyridine-3-carboxamide; 5-[(3AS,4R,6AR)-2-OXOHEXAHYDRO-1H-THIENO[3,4-D]IMIDAZOL-4-YL]PENTANOIC ACID; 5-[(3R)-3-(5-methoxy-2′,6′-dimethylbiphenyl-3-yl)but-1-yn-1-yl]-6-methylpyrimidine-2,4-diamine; {[2-(1h-1,2,3-Benzotriazol-1-Yl)-2-(3,4-Difluorophenyl)Propane-1,3-Diyl]Bis[4,1-Phenylene(Difluoromethylene)]}Bis(Phosphonic Acid); 5-[(3R)-3-(5-methoxy-4′-methylbiphenyl-3-yl)but-1-yn-1-yl]-6-methylpyrimidine-2,4-diamine; 5-[(3R)-3-(5-methoxybiphenyl-3-yl)but-1-yn-1-yl]-6-methylpyrimidine-2,4-diamine; 5-[(3S)-3-(2-methoxybiphenyl-4-yl)but-1-yn-1-yl]-6-methylpyrimidine-2,4-diamine; 5-[(4-AMINOCYCLOHEXYL)AMINO]-7-(PROPAN-2-YLAMINO)PYRAZOLO[1,5-A]PYRIMIDINE-3-CARBONITRILE; 4-O-(4,6-Dideoxy-4-{[4,5,6-Trihydroxy-3-(Hydroxymethyl)Cyclohex-2-En-1-Yl]Amino}-Beta-D-Lyxo-Hexopyranosyl)-Alpha-D-Erythro-Hexopyranose; 4-O-(4,6-Dideoxy-4-{[4-[(4-O-Hexopyranosylhexopyranosyl)Oxy]-5,6-Dihydroxy-3-(Hydroxymethyl)Cyclohex-2-En-1-Yl]Amino}Hexopyranosyl)Hexopyranose; 5-(4-METHYL-BENZOYLAMINO)-BIPHENYL-3,4′-DICARBOXYLIC ACID 3-DIMETHYLAMIDE-4′-HYDROXYAMIDE; {4-[3-(4-acetyl-3-hydroxy-2-propylphenoxy)propoxy]phenoxy}acetic acid; 6-(4-{(1S,2S)-2-AMINO-1-[(DIMETHYLAMINO)CARBONYL]-3-[(3S)-3-FLUOROPYRROLIDIN-1-YL]-3-OXOPROPYL}PHENYL)-1H-[1,2,4]TRIAZOLO[1,5-A]PYRIDIN-4-IUM; 5R-(3,4-DICHLOROPHENYLMETHYL)-3-(2-THIOPHENESULFONYLAMINO)-4-OXO-2-THIONOTHIAZOLIDINE; 6,7,8,9-TETRAHYDRO-4-HYDROXY-3-(1-PHENYLPROPYL)CYCLOHEPTA[B]PYRAN-2-ONE; 6,7-DIMETHOXY-4-[(3R)-3-(2-NAPHTHYLOXY)PYRROLIDIN-1-YL]QUINAZOLINE; 6,7-DIMETHOXY-4-[(3R)-3-(QUINOXALIN-2-YLOXY)PYRROLIDIN-1-YL]QUINAZOLINE; 6-(1,1-Dimethylallyl)-2-(1-Hydroxy-1-Methylethyl)-2,3-Dihydro-7h-Furo[3,2-G]Chromen-7-One; 6-(3,4-DIHYDROXYBENZYL)-3-ETHYL-1-(2,4,6-TRICHLOROPHENYL)-1H-PYRAZOLO[3,4-D]PYRIMIDIN-4(5H)-ONE; 6-(3′,5-DIMETHYLBENZYL)-1-ETHOXYMETHYL-5-ISOPROPYLURACIL; 1-((1R)-1-(HYDROXYMETHYL)-3-{6-[(3-PHENYLPROPANOYL)AMINO]-1H-INDOL-1-YL}PROPYL)-1H-IMIDAZOLE-4-CARBOXAMIDE; 6-(3-AMINOPROPYL)-4,9-DIMETHYLPYRROLO[3,4-C]CARBAZOLE-1,3(2H,6H)-DIONE; 6-(2,3,4,5,6,7-HEXAHYDRO-2,4,4-TRIMETHYL-1-METYLENEINDEN-2-YL)-3-METHYLHEXA-2,4-DIENOIC ACID; 1-((2-HYDROXYETHOXY)METHYL)-5-BENZYLPYRIMIDINE-2,4(1H,3H)-DIONE; 6-(2,6-DICHLOROPHENYL)-2-{[3-(HYDROXYMETHYL)PHENYL]AMINO}-8-METHYLPYRIDO[2,3-D]PYRIMIDIN-7(8H)-ONE; 6-(2,6-DIMETHOXYPHENYL)PYRIDO[2,3-D]PYRIMIDINE-2,7-DIAMINE; Adenosine Diphosphate 5-(Beta-Ethyl)-4-Methyl-Thiazole-2-Carboxylic Acid; 7-PYRIDIN-2-YL-N-(3,4,5-TRIMETHOXYPHENYL)-7H-PYRROLO[2,3-D]PYRIMIDIN-2-AMINE; 8-(2-CHLOROPHENYLAMINO)-2-(2,6-DIFLUOROPHENYLAMINO)-9-ETHYL-9H-PURINE-1,7-DIIUM; Adamantane-1-Carboxylic Acid-5-Dimethylamino-Naphthalene-1-Sulfonylamino-Butyl-Amide; Adamantane-1-Carboxylic Acid-5-Dimethylamino-Naphthalene-1-Sulfonylamino-Octyl-Amide; 6,6-DIMETHYL-1-[3-(2,4,5-TRICHLOROPHENOXY)PROPOXY]-1,6-DIHYDRO-1,3,5-TRIAZINE-2,4-DIAMINE; 1-(2,6-Dichlorophenyl)-5-(2,4-Difluorophenyl)-7-Piperidin-4-Yl-3,4-Dihydroquinolin-2(1h)-One; A-6619, [2h-Thieno[3,2-E]-1,2-Thiazine-6-Sulfonamide,2-(3-Hydroxyphenyl)-3-(4-Morpholinyl)-, 1,1-Dioxide]; A-6629, [2h-Thieno[3,2-E]-1,2-Thiazine-6-Sulfonamide,2-(3-Methoxyphenyl)-3-(4-Morpholinyl)-, 1,1-Dioxide]; Alyl-{4-[3-(4-Bromo-Phenyl)-Benzofuran-6-Yloxy]-but-2-Enyl}-Methyl-Amine; Alyl-{6-[3-(4-Bromo-Phenyl)-1-Methyl-1h-Indazol-6-Yl]Oxy}Hexyl)-N-Methylamine; Alyl-{6-[3-(4-Bromo-Phenyl)-Benzofuran-6-Yloxy]-Hexyl-}-Methyl-Amin; ALPHA-(2,6-DICHLOROPHENYL)-ALPHA-(2-ACETYL-5-METHYLANILINO)ACETAMIDE; 8-ethyl-3,10,10-trimethyl-4,5,6,8,10,12-hexahydropyrazolo[4′,3′:6,7]cyclohepta[1,2-b]pyrrolo[2,3-f]indol-9(1H)-one; 8-HYDROXY-2-OXA-BICYCLO[3.3.1]NON-6-ENE-3,5-DICARBOXYLIC ACID; 8-Benzyl-2-Hydroperoxy-2-(4-Hydroxy-Benzyl)-6-(4-Hydroxy-Phenyl)-2h-Imidazo[1,2-a]Pyrazin-3-One; Compound 12, N-Acetyl-4-[(Carboxycarbonyl)(2-Carboxyphenyl)Amino]-N-Pentyl-1-Napthylalaniamide; Compound 5, 2-(Naphthalen-1-Yl-Oxalyl-Amino)-Benzoicacid; N-(2-(((5-CHLORO-2-PYRIDINYL)AMINO)SULFONYL)PHENYL)-4-(2-OXO-1(2H)-PYRIDINYL)BENZAMIDE; CYCLOHEXYLMETHYL-2,3-DIHYDROXY-5-METHYL-HEXYLAMIDE; N-(1-CYANOCYCLOPROPYL)-3-({[(2S)-5-OXOPYRROLIDIN-2-YL]METHYL}SULFONYL)-N˜2˜-[(1S)-2,2,2-TRIFLUORO-1-(4-FLUOROPHENYL)ETHYL]-L-ALANINAMIDE; N-(1-ISOPROPYLPIPERIDIN-4-YL)-1-(3-METHOXYBENZYL)-1H-INDOLE-2-CARBOXAMIDE; Cyclohexyl-{4-[5-(3,4-Dichlorophenyl)-2-Piperidin-4-Yl-3-Propyl-3h-Imidazol-4-Yl]-Pyrimidin-2-Yl}Amine; N-(2-AMINOETHYL)-2-{3-CHLORO-4-[(4-ISOPROPYLBENZYL)OXY]PHENYL} ACETAMIDE; 1-(3-METHYLPHENYL)-1H-BENZIMIDAZOL-5-AMINE; N-(2,2,2-TRIFLUOROETHYL)-N-{4-[2,2,2-TRIFLUORO-1-HYDROXY-1-(TRIFLUOROMETHYL)ETHYL]PHENYL}BENZENESULFONAMIDE; N-(2-hydroxy-1,1-dimethylethyl)-1-methyl-3-(1H-pyrrolo[2,3-b]pyridin-2-yl)-1H-indole-5-carboxamide; 1-(4-IODOBENZOYL)-5-METHOXY-2-METHYLINDOLE-3-ACETIC ACID; N-(2-Morpholin-4-Yl-1-Morpholin-4-Ylmethyl-Ethyl)-3-Nitro-5-(3,4,5-Trihydroxy-6-Hydroxymethyl-Tetrahydro-Pyran-2-Yloxy)-Benzamide; N′-(3-CHLORO-4-METHOXY-PHENYL)-N-(3,4,5-TRIMETHOXYPHENYL)-1,3,5-TRIAZINE-2,4-DIAMINE; 1-(5-CHLORO-2,4-DIMETHOXYPHENYL)-3-(5-CYANOPYRAZIN-2-YL)UREA; N-(3-cyanophenyl)-2-methyl-5′-(5-methyl-1,3,4-oxadiazol-2-yl)-4-biphenylcarboxamide; N-(3-Cyclopropyl(5,6,7,8,9,10-Hexahydro-2-Oxo-2h-Cycloocta[B]Pyran-3-Yl)Methyl)Phenylbenzensulfonamide; N-(3-chlorophenyl)-N-methyl-2-oxo-3-[(3,4,5-trimethyl-1H-pyrrol-2-yl)methyl]-2H-indole-5-sulfonamide; N-(2-CHLORO-4-FLUOROBENZOYL)-N′-(5-HYDROXY-2-METHOXYPHENYL)UREA; N-(4-{4-AMINO-6-[4-(METHYLOXY)PHENYL]FURO[2,3-D]PYRIMIDIN-5-YL}PHENYL)-N′-[2-FLUORO-5-(TRIFLUOROMETHYL)PHENYL]UREA; N-(4-ACETYLPHENYL)-5-(5-CHLORO-2,4-DIHYDROXYPHENYL)-1H-PYRAZOLE-4-CARBOXAMIDE; 1-(5-ISOQUINOLINESULFONYL)-2-METHYLPIPERAZINE; N-(4-AMINO-5-CYANO-6-ETHOXYPYRIDIN-2-YL)-2-(4-BROMO-2,5-DIMETHOXYPHENYL)ACETAMIDE; N-(4-bromo-2-{[(3R,5S)-3,5-dimethylpiperidin-1-yl]carbonyl}phenyl)-4-morpholin-4-yl-4-oxobutanamide; N′-(5-CHLORO-1,3-BENZODIOXOL-4-YL)-N-(3,4,5-TRIMETHOXYPHENYL)PYRIMIDINE-2,4-DIAMINE; 1-(5′-Phospho-Beta-D-Ribofuranosyl)Barbituric Acid; 1-(5-Tert-Butyl-2-P-Tolyl-2h-Pyrazol-3-Yl)-3-[4-(2-Morpholin-4-Yl-Ethoxy)-Naphthalen-1-Yl]-Urea; N′-(5-CHLOROBENZOFURAN-2-CARBONYL)-2-(TRIFLUOROMETHYL)BENZENESULFONOHYDRAZIDE; 1-(CYCLOHEXYLAMINO)-3-(6-METHYL-3,4-DIHYDRO-1H-CARBAZOL-9(2H)-YL)PROPAN-2-OL; N-(8,9,10-Trihydroxy-7-Hydroxymethyl-2,4-Dioxo-6-Oxa-1,3-Diaza-Spiro[4.5]Dec-3-Yl-Acetamide; N-(TRANS-4-{(1 S,2S)-2-AMINO-3-[(3S)-3-FLUOROPYRROLIDIN-1-YL]-1-METHYL-3-OXOPROPYL}CYCLOHEXYL)-N-METHYLACETAMIDE; 1-(5-TERT-BUTYL-1,3,4-OXADIAZOL-2-YL)-2-(METHYLAMINO)ETHANONE; N-(CYCLOPROPYLMETHYL)-4-(METHYLOXY)-3-({5-[3-(3-PYRIDINYL)PHENYL]-1,3-OXAZOL-2-YL}AMINO)BENZENESULFONAMIDE; N,N-DIETHYL-2-[(2-THIENYLCARBONYL)AMINO]-4,5,6,7-TETRAHYDRO-1-BENZOTHIOPHENE-3-CARBOXAMIDE; 1-(thiophen-2-ylacetyl)-4-(3-thiophen-2-yl-1,2,4-oxadiazol-5-yl)piperidine; N-[(1S)-2-[(4-cyano-1-methylpiperidin-4-yl)amino]-1-(cyclohexylmethyl)-2-oxoethyl]morpholine-4-carboxamide; 1-(PHENYLMETHYL)CYCLOPENTYL[(1S)-1-FORMYLPENTYL]CARBAMATE; 1-(N-Imidazolyl)-2-Hydroxy-2-(2,3-Dichlorophenyl)Octane; N-(Allyloxycarbonyl)-4-[N-(Carboxy-Formyl)-2-(Benzoic Acid)-Amino]-L-Phenylalaninyl-Amino-Butyloxy-(6-Hydroxy-Benzoic Acid Methyl Ester); N-[(2R,3S)-3-AMINO-2-HYDROXY-4-PHENYLBUTYL]-4-METHOXY-2,3,6-TRIMETHYLBENZENESULFONAMIDE; N-[[2-METHYL-4-HYDROXYCARBAMOYL]BUT-4-YL-N]-BENZYL-P-[PHENYL]-P-[METHYL]PHOSPHINAMID; N-[[3-FLUORO-4-ETHOXY-PYRID-2-YL]ETHYL]-N′-[5-CHLORO-PYRIDYL]-THIOUREA; N-[[3-FLUORO-4-ETHOXY-PYRID-2-YL]ETHYL]-N′-[5-NITRILOMETHYL-PYRIDYL]-THIOUREA; N-[1-(AMINOMETHYL)CYCLOPROPYL]-3-(BENZYLSULFONYL)-N˜2˜-[(1S)-2,2,2-TRIFLUORO-1-(4-HYDROXYPHENYL)ETHYL]-L-ALANINAMIDE; 1,2,3,4-Tetrahydro-Isoquinoline-7-Sulfonic Acid Amide; 1,2-DI-1-(3,7,11,15-Tetramethyl-Hexadecane)-Sn-Glycerol; 1,2-Di-N-Pentanoyl-Sn-Glycero-3-Dithiophosphocholine; N-[(5R,14R)-5-AMINO-5,14-DIMETHYL-4-OXO-3-OXA-18-AZATRICYCLO[15.3.1.1˜7,11˜]DOCOSA-1(21),7(22),8,10,17,19-HEXAEN-19-YL]-N-METHYLMETHANESULFONAMIDE; N-[(2-AMINO-6-METHYLPYRIMIDIN-4-YL)METHYL]-3-{[(E)-(2-OXODIHYDROFURAN-3(2H)-YLIDENE)METHYL]AMINO}BENZENESULFONAMIDE; N-[1-Hydroxycarboxyethyl-Carbonyl]Leucylamino-2-Methyl-Butane; N-[2-(2,4-diaminopyrido[2,3-d]pyrimidin-7-yl)-2-methylpropyl]-4-phenoxybenzamide; N-[2-(4-AMINO-5,8-DIFLUORO-1,2-DIHYDROQUINAZOLIN-2-YL)ETHYL]-3-FURAMIDE; N-[2-(4-BROMOCINNAMYLAMINO)ETHYL]-5-ISOQUINOLINE SULFONAMIDE; N-[3-(Dimethylamino)Propyl]-2-({[4-({[4-(Formylamino)-1-Methyl-1h-Pyrrol-2-Yl]Carbonyl}Amino)-1-Methyl-1h-Pyrrol-2-Yl]Carbonyl}Amino)-5-Isopropyl-1,3-Thiazole-4-Carboxamide; N-[2-(6-AMINO-4-METHYLPYRIDIN-2-YL)ETHYL]-4-CYANOBENZAMIDE; 1,4-Dideoxy-5-Dehydro-02-Sulfo-Glucuronic Acid; N-[2-({[amino(imino)methyl]amino}oxy)ethyl]-2-{6-chloro-3-[(2,2-difluoro-2-phenylethyl)amino]-2-fluorophenyl}acetamide; N-[2-(1,3-BENZODIOXOL-5-YL)ETHYL]-1-[2-(1H-IMIDAZOL-1-YL)-6-METHYLPYRIMIDIN-4-YL]-D-PROLINAMIDE; 1-[(1s)-Carboxy-2-(Methylsulfinyl)Ethyl]-(3r)-[(5s)-5-Amino-5-Carboxypentanamido]-(4r)-Sulfanylazetidin-2-One; N-[2-(5-methyl-4H-1,2,4-triazol-3-yl)phenyl]-7H-pyrrolo[2,3-d]pyrimidin-4-amine; N-[3-(N′-HYDROXYCARBOXAMIDO)-2-(2-METHYLPROPYL)-PROPANOYL]-O-TYROSINE-N-METHYLAMIDE; N-[2(S)-Cyclopentyl-1(R)-Hydroxy-3(R)Methyl]-5-[(2(S)-Tertiary-Butylamino-Carbonyl)-4-(N1-(2)-(N-Methylpiperazinyl)-3-Chloro-Pyrazinyl-5-Carbonyl)-Piperazino]-4(S)-Hydroxy-2(R)-Phenylmethyl-Pentanamide; N-[3-[(1-Aminoethyl)(Hydroxy)Phosphoryl]-2-(1,1′-Biphenyl-4-Ylmethyl)Propanoyl]Alanine; N-[5-(1,1-DIOXIDOISOTHIAZOLIDIN-2-YL)-1H-INDAZOL-3-YL]-2-(4-PIPERIDIN-1-YLPHENYL)ACETAMIDE; 1-[(2R)-2-aminobutanoyl]-N-(4-carbamimidoylbenzyl)-L-prolinamide; N-[5′-O-Phosphono-Ribofuranosyl]-2-[2-Hydroxy-2-[4-[Glutamic Acid]-N-Carbonylphenyl]-3-[2-Amino-4-Hydroxy-Quinazolin-6-Yl]-Propanylamino]-Acetamide; 1-[(2S)-4-(5-phenyl-1H-pyrazolo[3,4-b]pyridin-4-yl)morpholin-2-yl]methanamine; N-[3-[[4-[(5-CHLORO-1,3-BENZODIOXOL-4-YL)AMINO]PYRIMIDIN-2-YL]AMINO]PHENYL]METHANESULFONAMIDE; N-[4-({[5-(DIMETHYLAMINO)-1-NAPHTHYL]SULFONYL}AMINO)BUTYL]-3-SULFANYLPROPANAMIDE; 1-[(2-NITROPHENYL)SULFONYL]-1H-PYRROLO[3,2-B]PYRIDINE-6-CARBOXAMIDE; N-[4-(AMINOSULFONYL)BENZYL]-5-(5-CHLORO-2,4-DIHYDROXYPHENYL)-1H-PYRAZOLE-4-CARBOXAMIDE; N-[4-(2-{2-[3-(2-Bromo-Acetylamino)-Propionylamino]-3-Hydroxy-Propionylamino}-Ethyl)-Phenyl]-Oxalamic Acid; N-[4-(2-CHLOROPHENYL)-1,3-DIOXO-1,2,3,6-TETRAHYDROPYRROLO[3,4-C]CARBAZOL-9-YL]FORMAMIDE; N-[4-(1-BENZOYLPIPERIDIN-4-YL)BUTYL]-3-PYRIDIN-3-YLPROPANAMIDE; N-{(1s)-4-[Bis(2-Chloroethyl)Amino]-1-Methylbutyl}-N-(6-Chloro-2-Methoxy-9-Acridinyl)Amine; N-{(1S,2R)-1-BENZYL-3-[(CYCLOPROPYLMETHYL)(2-FURYLSULFONYL)AMINO]-2-HYDROXYPROPYL}-N′-METHYLSUCCINAMIDE; N-{(1S,2R)-2-hydroxy-1-[(hydroxyamino)carbonyl]propyl}-4-{[4-(morpholin-4-ylmethyl)phenyl]ethynyl}benzamide; N-{(3R,4S)-4-[(6-amino-4-methylpyridin-2-yl)methyl]pyrrolidin-3-yl}-N′-(3-chlorobenzyl)ethane-1,2-diamine; N-{(2S)-3-[(1R)-1-aminoethyl](hydroxy)phosphoryl-2-benzylpropanoyl}-L-phenylalanine; N-{(3S,4S)-4-[(6-AMINO-4-METHYLPYRIDIN-2-YL)METHYL]PYRROLIDIN-3-YL}-N′-(4-CHLOROBENZYL)ETHANE-1,2-DIAMINE; 1-[2-(3-Biphenyl)-4-Methylvaleryl)]Amino-3-(2-Pyridylsulfonyl)Amino-2-Propanone; 1-[2-AMINO-2-CYCLOHEXYL-ACETYL]-PYRROLIDINE-3-CARBOXYLIC ACID 5-CHLORO-2-(2-ETHYLCARBAMOYL-ETHOXY)-BENZYLAMIDE; N-{(4s)-4-Amino-5-[(2-Aminoethyl)Amino]Pentyl}-N′-Nitroguanidine; N-{[(2R)-2,3-dihydroxypropyl]oxy}-3,4-difluoro-2-[(2-fluoro-4-iodophenyl)amino]benzamide; N-{[(2S,3S)-3-(ETHOXYCARBONYL)OXIRAN-2-YL]CARBONYL}-; N-{[(2S,3S)-3-(ETHOXYCARBONYL)OXIRAN-2-YL]CARBONYL}-L-ISOLEUCINE; N-{[(2S,3S)-3-(ETHOXYCARBONYL)OXIRAN-2-YL]CARBONYL}-L-ISOLEUCYL-L-ALANINE; N-{[(2S,3S)-3-(ETHOXYCARBONYL)OXIRAN-2-YL]CARBONYL}-L-ISOLEUCYL-L-ISOLEUCINE; 1-[(2-AMINO-4-CHLORO-5-METHYLPHENYL)SULFONYL]-L-PROLINE; N-[2-(1-Formyl-2-Methyl-Propyl)-1-(4-Piperidin-1-Yl-but-2-Enoyl)-Pyrrolidin-3-Yl]-Methanesulfonamide; N-[(2R,3S)-3-AMINO-2-HYDROXY-4-PHENYLBUTYL]NAPHTHALENE-2-SULFONAMIDE; N-(4-CARBAMIMIDOYL-3-CHORO-PHENYL)-2-HYDROXY-3-IODO-5-METHYL-BENZAMIDE; N-(4-{[(3S)-3-(dimethylamino)pyrrolidin-1-yl]carbonyl}phenyl)-5-fluoro-4-[2-methyl-1-(1-methylethyl)-1H-imidazol-5-yl]pyrimidin-2-amine; Cyclopropyl-{4-[5-(3,4-Dichlorophenyl)-2-[(1-Methyl)-Piperidin]-4-Yl-3-Propyl-3h-Imidazol-4-Yl]-Pyrimidin-2-Yl}Amine; BUT-3-ENYL-[5-(4-CHLORO-PHENYL)-3,6-DIHYDRO-[1,3,4]THIADIAZIN-2-YLIDENE]-AMINE; Cp-166572, 2-Hydroxymethyl-4-(4-N,N-Dimethylaminosulfonyl-1-Piperazino)-Pyrimidine; 1-{(2S,5S)-4-FLUORO-5-[(TRITYLOXY)METHYL]TETRAHYDROFURAN-2-YL}PYRIMIDINE-2,4(1H,3H)-DIONE; Cp403700, (S)-1-{2-[(5-Chloro-1h-Indole-2-Carbonyl)-Amino]-3-Phenyl-Propionyl}-Azetidine-3-Carboxylate; DIETHYL (1R,2S,3R,4S)-5,6-BIS(4-HYDROXYPHENYL)-7-OXABICYCLO[2.2.1]HEPT-5-ENE-2,3-DICARBOXYLATE; Delta-Bis(2,2′-Bipyridine)-(5-Methyl-2-2′-Bipyridine)-C2-Adamantane Ruthenium (Ii); Delta-Bis(2,2′-Bipyridine)-(5-Methyl-2-2′-Bipyridine)-C9-Adamantane Ruthenium (Ii); 1-{[1-(2-AMINO-3-PHENYL-PROPIONYL)-PYRROLIDINE-2-CARBONYL]-AMINO}-2-(3-CYANO-PHENYL)-ETHANEBORONIC ACID; D-phenylalanyl-N-[(1S)-4-{[amino(iminio)methyl]amino}-1-(chloroacetyl)butyl]-L-prolinamide; DIMETHYL-(4,5,6,7-TETRABROMO-1H-BENZOIMIDAZOL-2-YL)-AMINE; ethyl 3-[(E)-2-amino-1-cyanoethenyl]-6,7-dichloro-1-methyl-1H-indole-2-carboxylate; ETHYL 4-[(4-CHLOROPYRIDIN-2-YL)AMINO]PIPERIDINE-1-CARBOXYLATE; ETHYL 4-[(4-METHYLPYRIDIN-2-YL)AMINO]PIPERIDINE-1-CARBOXYLATE; difluoro(5-{2-[(5-octyl-1H-pyrrol-2-yl-kappaN)methylidene]-2H-pyrrol-5-yl-kappaN}pentanoato)boron; 10,11-dimethoxy-4-methyldibenzo[c,f]-2,7-naphthyridine-3,6-diamine; METHYL (2Z)-2-(2-{[6-(2-CYANOPHENOXY)PYRIMIDIN-4-YL]OXY}PHENYL)-3-METHOXYACRYLATE; 1-{[(3R)-3-methyl-4-({4-[(1S)-2,2,2-trifluoro-1-hydroxy-1-methyl ethyl]phenyl}sulfonyl)piperazin-1-yl]methyl}cyclopropanecarboxamide; METHYL (3R)-3-{[(3R)-3-{[(3R)-3-HYDROXYBUTANOYL]OXY}BUTANOYL]OXY}BUTANOATE; 11-TRANS-13-TRANS-15-CIS-OCTADECATRIENOIC ACID; methyl [(1E,5R)-5-{(3S)-3-[(2E,4E)-2,5-dimethylocta-2,4-dienoyl]-2,4-dioxo-3,4-dihydro-2H-pyran-6-yl}hexylidene]carbamate; METHYL 1-(4-{[(2,4-DIAMINOPTERIDIN-6-YL)METHYL](METHYL)AMINO}BENZOYL)PIPERIDINE-4-CARBOXYLATE; METHYL 1-(4-{[(2,4-DIAMINOPTERIDIN-6-YL)METHYL]AMINO}BENZOYL)PIPERIDINE-4-CARBOXYLATE; 1-Azepan-1-Yl-2-Phenyl-2-(4-Thioxo-1,4-Dihydro-Pyrazolo[3,4-D]Pyrimidin-5-Yl)Ethanone Adduct; METHYL 4-{[({[(2R,5S)-5-{[(2S)-2-(AMINOMETHYL)PYRROLIDIN-1-YL]CARBONYL}PYRROLIDIN-2-YL]METHYL}AMINO)CARBONYL]AMINO}BENZOATE; METHYL N-({(2S,3S)-3-[(PROPYLAMINO)CARBONYL]OXIRAN-2-YL}CARBONYL)-L-ISOLEUCYL-L-PROLINATE; METHYL N-[(2′,4′-DIFLUORO-4-HYDROXY-5-IODOBIPHENYL-3-YL)CARBONYL]-BETA-ALANINATE; 17Alpha-methyl-3beta,17beta-dihydroxyandrost-4-ene; METHYL N-[(2S,3R)-3-AMINO-2-HYDROXY-3-(4-METHYLPHENYL)PROPANOYL]-D-ALANYL-D-LEUCINATE; Mono-[3,4-Dihydroxy-5-(5-Methyl-Benzoimidazol-1-Yl)-Tetrahydor-Furan-2-Ylmethyl] Ester; Morpholine-4-Carboxylic Acid (1-(3-Benzenesulfonyl-1-Phenethylallylcarbamoyl)-3-Methylbutyl)-Amide; Morpholine-4-Carboxylic Acid [1-(2-Benzylsulfanyl-1-Formyl-Ethylcarbamoyl)-2-Phenyl-Ethyl]-Amide; Morpholine-4-Carboxylic Acid [1s-(2-Benzyloxy-1r-Cyano-Ethylcarbamoyl)-3-Methyl-Butyl]Amide; 1-Amino-2,3-Dihydroxy-5-Hydroxymethyl Cyclohex-5-Ene; N-((1R,2R)-2-(5-CHLORO-1H-INDOLE-2-CARBOXAMIDO)CYCLOHEXYL)-5-METHYL-4,5,6,7-TETRAHYDROTHIAZOLO[5,4-C]PYRIDINE-2-CARBOXAMIDE; 1-butanoyl-N-(4-carbamimidoylbenzyl)-L-prolinamide; N-((2-aminopyrimidin-5-yl)methyl)-5-(2,6-difluorophenyl)-3-ethylpyrazolo[1,5-a]pyrimidin-7-amine; N′-((2S,3R)-3-AMINO-2-HYDROXY-5-(ISOPROPYLSULFANYL)PENTANOYL)-N-3-CHLOROBENZOYL HYDRAZIDE; 1-BENZYL-4-[(5,6-DIMETHOXY-1-INDANON-2-YL)METHYL]PIPERIDINE; N-({(2S)-1-[(3R)-3-AMINO-4-(2-FLUOROPHENYL)BUTANOYL]PYRROLIDIN-2-YL}METHYL)BENZAMIDE; N-({(3R,4R)-4-[(benzyloxy)methyl]pyrrolidin-3-yl}methyl)-N-(2-methylpropyl)benzenesulfonamide; N-({(2S,3S)-3-[(BENZYLAMINO)CARBONYL]OXIRAN-2-YL}CARBONYL)-L-ISOLEUCYL-L-PROLINE; N-{1-[5-(1-Carbamoyl-2-Mercapto-Ethylcarbamoyl)-Pentylcarbamoyl]-2-[4-(Difluoro-Phosphono-Methyl)-Phenyl]-Ethyl}-3-{2-[4-(Difluoro-Phosphono-Methyl)-Phenyl]-Acetylamino}-Succinamic Acid; N-({[4-(AMINOSULFONYL)PHENYL]AMINO}CARBONYL)-4-METHYLBENZENESULFONAMIDE; N-({4-[(2-aminopyridin-4-yl)oxy]-3-fluorophenyl}carbamoyl)-2-(4-fluorophenyl)acetamide; 1-Carboxyethylaminomethyl-4-Aminomethylbenzene; N-({6-[(4-CYANOBENZYL)OXY]NAPHTHALEN-2-YL}SULFONYL)-D-GLUTAMIC ACID; N,N-DIMETHYL(5-(PYRIDIN-3-YL)FURAN-2-YL)METHANAMINE; ISOPROPYL (2S)-2-ETHYL-7-FLUORO-3-OXO-3,4-DIHYDROQUINOXALINE-1(2H)-CARBOXYLATE; ISOQUINOLINE-5-SULFONIC ACID (2-(2-(4-CHLOROBENZYLOXY)ETHYLAMINO)ETHYL)AMIDE; N,N-DIMETHYL-4-(4-PHENYL-1H-PYRAZOL-3-YL)-1H-PYRROLE-2-CARBOXAMIDE; N,N′-DIMETHYL-N-(ACETYL)-N′-(7-NITROBENZ-2-OXA-1,3-DIAZOL-4-YL)ETHYLENEDIAMINE; N,N′-DIPHENYLPYRAZOLO[1,5-A][1,3,5]TRIAZINE-2,4-DIAMINE; 1-Deoxy-1-Acetylamino-Beta-D-Gluco-2-Heptulopyranosonamide; N-[(1-CHLORO-4-HYDROXYISOQUINOLIN-3-YL)CARBONYL]GLYCINE; N-(1,4-DIHYDRO-5H-TETRAZOL-5-YLIDENE)-9-OXO-9H-XANTHENE-2-SULFONAMIDE; N-(1-BENZYL-3,3,3-TRIFLUORO-2,2-DIHYDROXY-PROPYL)-ACETAMIDE; METHYL(2-ACETOXY-2-(2-CARBOXY-4-AMINO-PHENYL))ACETATE; 1-GUANIDINO-4-(N-PHENYLMETHANESULFONYL-L-LEUCYL-L-PROLYLAMINO)BUTANE; 8-Benzyl-2-Hydroxy-2-(4-Hydroxy-Benzyl)-6-(4-Hydroxy-Phenyl)-2h-Imidazo[1,2-a]Pyrazin-3-One; 1-Monohexanoyl-2-Hydroxy-Sn-Glycero-3-Phosphate; 9-(4-HYDROXY-2,6-DIMETHYL-PHENYL)-3,7-DIMETHYL-NONA-4,6,8-TRIENOIC ACID; 9-HYDROXY-6-(3-HYDROXYPROPYL)-4-(2-METHOXYPHENYL)PYRROLO[3,4-C]CARBAZOLE-1,3(2H,6H)-DIONE; 1-N-(4-SULFAMOYLPHENYL-ETHYL)-2,4,6-TRIMETHYLPYRIDINIUM; Benzofuran-2-Carboxylic Acid {(S)-3-Methyl-1-[3-Oxo-1-(Pyridin-2-Ylsulfonyl)Azepan-4-Ylcarbamoyl]Butyl}Amide; BENZYL N-({(2S,3S)-3-[(PROPYLAMINO)CARBONYL]OXIRAN-2-YL}CARBONYL)-L-ISOLEUCYL-L-PROLINATE; BIS-1,2-{[(Z)-2-CARBOXY-2-METHYL-1,3-DIOXANE]-5-YLOXYCARBAMOYL}-ETHANE; Cis-[4,5-Bis-(4-Chlorophenyl)-2-(2-Isopropoxy-4-Methoxyphenyl)-4,5-Dihyd Roimidazol-1-Yl]-Piperazin-1-Yl-Methanone; Cis-[4,5-Bis-(4-Bromophenyl)-2-(2-Ethoxy-4-Methoxyphenyl)-4,5-Dihydroimidazol-1-Yl]-[4-(2-Hydroxyethyl)Piperazin-1-Yl]Methanone; 1-PHENYL-1H-PYRAZOLE-4-CARBOXYLIC ACID; BIS-1,2-{[(Z)-2CARBOXY-2-METHYL-1,3-DIOXANE]-5-YLOXYCARBONYL}-PIPERAZINE; CIS-4-METHYL-N-[(1S)-3-(METHYLSULFANYL)-1-(PYRIDIN-4-YLCARBAMOYL)PROPYL]CYCLOHEXANECARBOXAMIDE; 8-bromo-4-(2-chlorophenyl)-N-(2-hydroxyethyl)-6-methyl-1,3-dioxo-1,2,3,6-tetrahydropyrrolo[3,4-e]indole-7-carboxamide; N{[6-(PENTYLOXY)NAPHTHALEN-2-YL]SULFONYL}-D-GLUTAMIC ACID; N-{1-[(1-carbamoylcyclopropyl)methyl]piperidin-4-yl}-N-cyclopropyl-4-[(1S)-2,2,2-trifluoro-1-hydroxy-1-methylethyl]benzamide; CARBOBENZYLOXY-(L)-LEUCINYL-(L)LEUCINYL METHOXYMETHYLKETONE; 1-O-Octyl-2-Heptylphosphonyl-Sn-Glycero-3-Phosphoethanolamine; N-{2,4-difluoro-3-[(5-pyridin-3-yl-1H-pyrrolo[2,3-b]pyridin-3-yl)carbonyl]phenyl}ethanesulfonamide; 1-Methyl-3-Oxo-1,3-Dihydro-Benzo[C]Isothiazole-5-Sulfonic Acid Amide; N-{2′-[(4-FLUOROPHENYL)AMINO]-4,4′-BIPYRIDIN-2-YL}-4-METHOXYCYCLOHEXANECARBOXAMIDE; 2-((3′,5′-DIMETHOXY-4′-HYDROXYPHENYL)AZO)BENZOIC ACID; N-{2-[6-(2,4-DIAMINO-6-ETHYLPYRIMIDIN-5-YL)-2,2-DIMETHYL-3-OXO-2,3-DIHYDRO-4H-1,4-BENZOTHIAZIN-4-YL]ETHYL}ACETAMIDE; N-{2-methyl-5-[(6-phenylpyrimidin-4-yl)amino]phenyl}methanesulfonamide; N-{2-[4-(AMINOSULFONYL)PHENYL]ETHYL}ACETAMIDE; N″-{3-[(3s,8ar)-1,4-Dioxooctahydropyrrolo[1,2-a]Pyrazin-3-Yl]Propyl}Guanidine; N-{3-[(4-{[3-(TRIFLUOROMETHYL)PHENYL]AMINO}PYRIMIDIN-2-YL)AMINO]PHENYL}CYCLOPROPANECARBOXAMIDE; N-{3-[4-(3-Amino-Propyl)-Piperazin-1-Yl]-Propyl}-3-(2-Thiophen-2-Yl-Acetylamino)-5-(3,4,5-Trihydroxy-6-Hydroxymethyl-Tetrahydro-Pyran-2-Yloxy)-Benzamide; N-{3-[(7ar,12as,12bs)-7-Oxo-1,3,4,6,7,7a,12a,12b-Octahydroindolo[2,3-a]Quinolizin-12(2h)-Yl]Propyl}Propane-2-Sulfonamide; N-{3-[(5-chloro-1H-pyrrolo[2,3-b]pyridin-3-yl)carbonyl]-2,4-difluorophenyl}propane-1-sulfonamide; 2-({2-[(3R)-3-AMINOPIPERIDIN-1-YL]-4-OXOQUINAZOLIN-3(4H)-YL}METHYL)BENZONITRILE; N-{3-[4-(3-Amino-Propyl)-Piperazin-1-Yl]-Propyl}-3-Nitro-5-(Galactopyranosyl)-Beta-Benzamide; N-{3-[4-hydroxy-1-(3-methylbutyl)-2-oxo-1,2-dihydropyrrolo[1,2-b]pyridazin-3-yl]-1,1-dioxido-2H-1,2,4-benzothiadiazin-7-yl}methanesulfonamide; N-{3-[5-(1H-1,2,4-triazol-3-yl)-1H-indazol-3-yl]phenyl}furan-2-carboxamide; N-{3-[5-(6-Amino-Purin-9-Yl)-3,4-Dihydroxy-Tetrahydro-Furan-2-Yl]-Allyl}-2,3-Dihydroxy-5-Nitro-Benzamide; N-{5-[4-(4-METHYLPIPERAZIN-1-YL)PHENYL]-1H-PYRROLO[2,3-B]PYRIDIN-3-YL}NICOTINAMIDE; N-{4-METHYL-3-[(3-PYRIMIDIN-4-YLPYRIDIN-2-YL)AMINO]PHENYL}-3-(TRIFLUOROMETHYL)BENZAMIDE; N-{3-METHYL-5-[2-(PYRIDIN-4-YLAMINO)-ETHOXY]-PHENYL}-BENZENESULFONAMIDE; 2-(2-{2-[(BIPHENYL-4-YLMETHYL)-AMINO]-3-MERCAPTO-PENTANOYLAMINO}-ACETYLAMINO)-3-METHYL-BUTYRIC ACID METHYL ESTER; N˜2˜-(biphenyl-4-ylsulfonyl)-N-hydroxy-N˜2˜-(2-hydroxyethyl)glycinamide; 2-(2,6-DICHLOROPHENYL)-1,3-BENZOXAZOLE-6-CARBOXYLIC ACID; N˜2˜-1,3-BENZOXAZOL-2-YL-3-CYCLOHEXYL-N-[2-[(4-METHOXYPHENYL)AMINO]ETHYL]-L-ALANINAMIDE; N˜2˜-1H-benzimidazol-5-yl-N˜4˜-(3-cyclopropyl-1H-pyrazol-5-yl)pyrimidine-2,4-diamine; N˜3˜-[5-(1H-INDOL-6-YL)-2-(PYRIDIN-2-YLMETHOXY)BENZYL]PYRIDINE-2,3-DIAMINE; 2-(2,6-DIFLUOROPHENOXY)-N-(2-FLUOROPHENYL)-9-ISOPROPYL-9H-PURIN-8-AMINE; ˜-4˜-(3-methyl-1H-indazol-6-yl)-N˜2˜-(3,4,5-trimethoxyphenyl)pyrimidine-2,4-diamine; N˜4˜-methyl-N˜4˜-(3-methyl-1H-indazol-6-yl)-N˜2˜-(3,4,5-trimethoxyphenyl)pyrimidine-2,4-diamine; N˜6˜-cyclohexyl-N˜2˜-(4-morpholin-4-ylphenyl)-9H-purine-2,6-diamine; N1-(1-Dimethylcarbamoyl-2-Phenyl-Ethyl)-2-Oxo-N4-(2-Pyridin-2-Yl-Ethyl)-Succinamide; N1-(2-AMINO-4-METHYLPENTYL)OCTAHYDRO-PYRROLO[1,2-A] PYRIMIDINE; N-1,2,3,4-Tetrahydronaphth-1-Yl-2′-[3,5-Dimethoxybenzamido]-2′-Deoxy-Adenosine; N1,N14-Bis((S-Methyl)Isothioureido)Tetradecane; N1,N2-ETHYLENE-2-METHYLAMINO-4,5,6,7-TETRABROMO-BENZIMIDAZOLE; N1-CYCLOPENTYL-N2-(THIAZOL-2-YL)OXALAMIDE; N-2-Thiophen-2-Yl-Acetamide Boronic Acid; N-1H-indazol-5-yl-2-(6-methylpyridin-2-yl)quinazolin-4-amine; N2-({[(4-Bromophenyl)Methyl]Oxy}Carbonyl)-N1-[(1s)-1-Formylpentyl]-L-Leucinamide; N2-[(benzyloxy)carbonyl]-n1-[(3S)-1-cyanopyrrolidin-3-yl]-l-leucinamide; N-1H-imidazol-2-yl-N′-[4-(1H-imidazol-2-ylamino)phenyl]benzene-1,4-diamine; N6-ISOPENTENYL-ADENOSINE-5′-MONOPHOSPHATE; 2-(2-PHENYL-3-PYRIDIN-2-YL-4,5,6,7-TETRAHYDRO-2H-ISOPHOSPHINDOL-1-YL)PYRIDINE; N4-(N,N-DIPHENYLCARBAMOYL)-AMINOGUANIDINE; N-ETHYL-N-ISOPROPYL-3-METHYL-5-{[(2S)-2-(PYRIDIN-4-YLAMINO)PROPYL]OXY}BENZAMIDE; N-Hydroxy 1n(4-Methoxyphenyl)Sulfonyl-4-(Z,E-N-Methoxyimino)Pyrrolidine-2r-Carboxamide; N7-BUTYL-N2-(5-CHLORO-2-METHYLPHENYL)-5-METHYL[1,2,4]TRIAZOLO[1,5-A]PYRIMIDINE-2,7-DIAMINE; N-Hydroxy-4-[(4-Methoxylphenyl)Sulfonyl]-2,2-Dimethyl-Hexahydro-1,4-Thiazepine-3(S)-Carboxamide; N-HYDROXY-2-[4-(4-PHENOXY-BENZENESULFONYL)-TETRAHYDRO-PYRAN-4-YL]-ACETAMIDE; N-hydroxy-4-({4-[4-(trifluoromethyl)phenoxy]phenyl}sulfonyl)tetrahydro-2H-pyran-4-carboxamide; N-HYDROXY-2(R)-[[(4-METHOXYPHENYL)SULFONYL](3-PICOLYL)AMINO]-3-METHYLBUTANAMIDE HYDROCHLORIDE; N-Hydroxy-4-(Methyl{[5-(2-Pyridinyl)-2-Thienyl]Sulfonyl}Amino)Benzamide; N-hydroxy-5-[(3-phenyl-5,6-dihydroimidazo[1,2-a]pyrazin-7(8H)-yl)carbonyl]thiophene-2-carboxamide; N4-HYDROXY-2-ISOBUTYL-N1-(9-OXO-1,8-DIAZA-TRICYCLO[10.6.1.013,18]NONADECA-12(19),13,15,17-TETRAEN-10-YL)-SUCCINAMIDE; N6-(2,5-Dimethoxy-Benzyl)-N6-Methyl-Pyrido[2,3-D]Pyrimidine-2,4,6-Triamine; N-Hydroxy-1-(4-Methoxyphenyl)Sulfonyl-4-Benzyloxycarbonyl-Piperazine-2-Carboxamide; N-ACETYL-L-PHENYLALANYL-4-[DIFLUORO(PHOSPHONO)METHYL]-L-PHENYLALANINAMIDE; NALPHA-[(BENZYLOXY)CARBONYL]-N-[(1R)-4-HYDROXY-1-METHYL-2-OXOBUTYL]-L-PHENYLALANINAMIDE; N-METHYLALANYL-3-METHYLVALYL-4-PHENOXY-N-(1,2,3,4-TETRAHYDRONAPHTHALEN-1-YL)PROLINAMIDE; N-Alpha-(2-Naphthylsulfonyl)-N(3-Amidino-L-Phenylalaninyl)-4-Acetyl-Piperazine; N-Alpha-(2-Naphthylsulfonyl)-N-(3-Amidino-L-Phenylalaninyl)-D-Pipecolinic Acid; N-Alpha-(2-Naphthylsulfonyl)-N(3-Amidino-L-Phenylalaninyl)Isopipecolinic Acid Methyl Ester; 2-(4-HYDROXY-3-NITROPHENYL)ACETIC ACID; Nadph Dihydro-Nicotinamide-Adenine-Dinucleotidephosphate; N-anthracen-2-yl-5-methyl[1,2,4]triazolo[1,5-a]pyrimidin-7-amine; N-BENZYL-4-[4-(3-CHLOROPHENYL)-1H-PYRAZOL-3-YL]-1H-PYRROLE-2-CARBOXAMIDE; 2-(4-CHLOROBENZYLAMINO)-4-(PHENYLAMINO)PYRAZOLO[1,5-A][1,3,5]TRIAZINE-8-CARBONITRILE; N-BENZYLOXYCARBONYL-L-SERINE-BETALACTONE; N-ISOBUTYL-N-[4-METHOXYPHENYLSULFONYL]GLYCYL HYDROXAMIC ACID; Nalpha-(2-Naphthylsulfonylglycyl)-3-Amidino-D,L-Phenylalanine-Isopropylester; N-METHYL(5-(PYRIDIN-3-YL)FURAN-2-YL)METHANAMINE; PHOSPHONIC ACID 2-DODECANOYLAMINO-HEXYL ESTER PROPYL ESTER; 2-(4-METHYLPHENOXY)ETHYLPHOSPHINATE; Phosphoric Acid Mono-[3,4-Dihydroxy-5-(5-Hydroxy-Benzoimidazol-1-Yl)Tetrahydro-Furan-2-Ylmethyl] Ester; 2-(5-CHLORO-2-THIENYL)-N-{(3S)-1-[(1S)-1-METHYL-2-MORPHOLIN-4-YL-2-OXOETHYL]-2-OXOPYRROLIDIN-3-YL}ETHANESULFONAMIDE; 2-(6-HYDROXY-1,3-BENZOTHIAZOL-2-YL)-1,3-THIAZOL-4(5H)-ONE; N-METHYL-{4-[2-(7-OXO-6,7-DIHYDRO-8H-[1,3]THIAZOLO[5,4-E]INDOL-8-YLIDENE)HYDRAZINO]PHENYL}METHANESULFONAMIDE; N-Butyl-11-[(7r,8r,9s,13s,14s,17s)-3,17-Dihydroxy-13-Methyl-7,8,9,11,12,13,14,15,16,17-Decahydro-6h-Cyclopenta[a]Phenanthren-7-Yl]-N-Methylundecanamide; N-METHYL-4-{[(2-OXO-1,2-DIHYDRO-3H-INDOL-3-YLIDENE)METHYL]AMINO}BENZENESULFONAMIDE; O-(((1R)-((N-(PHENYL-METHOXY-CARBONYL)-ALANYL)-AMINO)METHYL)HYDROXYPHOSPHINYL)3-L-PHENYLLACTATE; O-(((1R)-((N-PHENYLMETHOXYCARBONYL-L-ALANYL)AMINO)ETHYL)HYDROXYPHOSPHONO)-L-BENZYLACETIC ACID; 2-(3-Methoxyphenyl) Benzimidazole-4-Carboxamide; Uridine-5′-Monophosphate 2-Deoxy-2-Fluoro-Galactopyranosyl-Monophosphate Ester; 2-(6-methylpyridin-2-yl)-N-pyridin-4-ylquinazolin-4-amine; PYRIMIDINE-4,6-DICARBOXYLIC ACID BIS-(3-METHYL-BENZYLAMIDE); PYRIMIDINE-4,6-DICARBOXYLIC ACID BIS-(4-FLUORO-3-METHYL-BENZYLAMIDE); PYRIMIDINE-4,6-DICARBOXYLIC ACID BIS-[(PYRIDIN-3-YLMETHYL)-AMIDE]; R-2-{[4′-Methoxy-(1,1′-Biphenyl)-4-Yl]-Sulfonyl}-Amino-6-Methoxy-Hex-4-Ynoic Acid; R-3-FLUORO-4-[2-HYDROXY-2-(5,5,8,8-TETRAMETHYL-5,6,7,8,-TETRAHYDRO-NAPHTALEN-2-YL)-ACETYLAMINO]-BENZOIC ACID; REL-(9R,12S)-9,10,11,12-TETRAHYDRO-9,12-EPOXY-1H-DIINDOLO[1,2,3-FG:3′,2′,1′-KL]PYRROLO[3,4-I][1,6]BENZODIAZOCINE-1,3(2H)-DIONE; N-Methyl-N-(10-Methylundecanoyl)-D-Seryl-L-Alanyl-N˜1˜-[(7s,10s,13s)-13-Carboxy-3,18-Di hydroxy-10-Methyl-8,11-Dioxo-9, 12-Diazatricyclo[13.3.1.1˜2,6˜]Icosa-1(19),2(20),3, 5, 15,17-Hexaen-7-YI]-N˜1˜-Methylglycinamide; O5′-(4-(3-{2-[2-((R)-3- Hydroxy-4-(Trimethylammonio)-1-Oxo-Butyl)Sulfanyl-Ethylcarbamoyl]-Ethylcarbamoyl}-(R)-3-Hydroxy-2,2-Dimethyl-Propyl)-1-Hydroxy-3-Oxido-1,3-Dioxo-2,4-Dioxa-1,3-Diphosphabut-1-Yl) 3-Phospho-Adenosine; Phosporic Acid Mono-[3,4-Dihydroxy-5-(5-Methoxy-Benzoimidazol-1-Yl)-Tetrahydro-Furan-2-Ylmethyl] Ester; 2-(hydrazinocarbonyl)-3-phenyl-1H-indole-5-sulfonamide; PHENYLALANYLAMINODI(ETHYLOXY)ETHYL BENZENESULFONAMIDEAMINOCARBONYLBENZENESULFONAMIDE; O6-(R)-ROSCOVITINE, R-2-(6-BENZYLOXY-9-ISOPROPYL-9H-PURIN-2-YLAMINO)-BUTAN-1-OL; N-cyclopropyl-3-{[1-(2,4-difluorophenyl)-7-methyl-6-oxo-6,7-dihydro-1H-pyrazolo[3,4-b]pyridin-4-yl]amino}-4-methylbenzamide; N-cyclopropyl-N-(trans-4-pyridin-3-ylcyclohexyl)-4-[(1S)-2,2,2-trifluoro-1-hydroxy-1-methylethyl]benzamide; 2-(methylamino)-N-(4-methyl-1,3-thiazol-2-yl)-5-[(4-methyl-4H-1,2,4-triazol-3-yl)sulfanyl]benzamide; 2-(Biphenyl-4-Sulfonyl)-1,2,3,4-Tetrahydro-Isoquinoline-3-Carboxylic Acid; tert-butyl [(2-amino-4-oxo-4,7-dihydro-3H-pyrrolo[2,3-d]pyrimidin-5-yl)methyl]carbamate; TERT-BUTYL [(1R)-2-METHYL-1-(1,3,4-OXADIAZOL-2-YL)PROPYL]CARBAMATE; TERT-BUTYL [(1S)-2-METHYL-1-(1,3,4-OXADIAZOL-2-YL)PROPYL]CARBAMATE; S-(2-{[N-(2-HYDROXY-4-{[HYDROXY(OXIDO)PHOSPHINO]OXY}-3,3-DIMETHYLBUTANOYL)-BETA-ALANYL]AMINO}ETHYL) HEXANETHIOATE; 2,2′-{[9-(HYDROXYIMINO)-9H-FLUORENE-2,7-DIYL]BIS(OXY)}DIACETIC ACID; THIENO[3,2-B]PYRIDINE-2-SULFONIC ACID [1-(1-AMINO-ISOQUINOLIN-7-YLMETHYL)-2-OXO-PYRROLDIN-3-YL]-AMIDE; THIENO[3,2-B]PYRIDINE-2-SULFONIC ACID [2-OXO-1-(1H-PYRROLO[2,3-C]PYRIDIN-2-YLMETHYL)-PYRROLIDIN-3-YL]-AMIDE; THIOPHENE-2,5-DISULFONIC ACID 2-AMIDE-5-(4-METHYL-BENZYLAMIDE); TRANS-(1S,2S)-2-AMINO-1,2,3,4-TETRAHYDRONAPHTHALEN-1-OL; TRANS-2-(DIMETHYLPHENYLSILYL)-PIPERIDINE-N-OXIDE; 2′,3-Dehydro-2′,3′-Deoxy-Thymidine 5′-Diphosphate; TERT-BUTYL 4-({[4-(BUT-2-YN-1-YLAMINO)PHENYL]SULFONYL}METHYL)-4-[(HYDROXYAMINO)CARBONYL]PIPERIDINE-1-CARBOXYLATE; tert-butyl 4-(3-thiophen-2-yl-1,2,4-oxadiazol-5-yl)piperidine-1-carboxylate; Spiro(2,4,6-Trinitrobenzene[1,2a]-20′,3d-Methylene-Adenine-Triphosphate; 2-(BUTYRYLOXY)-1-{[(TETRAHYDROXYPHOSPHORANYL)OXY]METHYL}ETHYL BUTYRATE; Sulfamic Acid 2,3-O-(1-Methylethylidene)-4,5-O-Sulfonyl-Beta-Fructopyranose Ester; S-[(2E)-3,7-DIMETHYLOCTA-2,6-DIENYL] TRIHYDROGENTHIODIPHOSPHATE; 2,4-Diamino-6-Phenyl-5,6,7,8,-Tetrahydropteridine; S-[2-({N-[(2S)-2-hydroxy-3,3-dimethyl-4-(phosphonooxy)butanoyl]-beta-alanyl}amino)ethyl] octanethioate; S-[3-(3,4-DICHLOROPHENYL)-3-OXOPROPYL]-L-CYSTEINE; S-[5-(TRIFLUOROMETHYL)-4H-1,2,4-TRIAZOL-3-YL] 5-(PHENYLETHYNYL)FURAN-2-CARBOTHIOATE; S-{2-[(2-chloro-4-sulfamoylphenyl)amino]-2-oxoethyl} 6-methyl-3,4-dihydroquinoline-1(2H)-carbothioate; S-{2-[Amino(Dihydroxy)-Lambda-4--Sulfanyl]Ethyl}-D-Cysteine; S-{3-[(4-ANILINOQUINAZOLIN-6-YL)AMINO]-3-OXOPROPYL}-L-CYSTEINE; S-3-(4-FLUOROPHENOXY)-2-HYDROXY-2-METHYL-N-[4-NITRO-3-(TRI FLUOROMETHYL)PHENYL]PROPANAMIDE; octyl 3-amino-3-deoxy-2-O-(2,6-dideoxy-alpha-L-lyxo-hexopyranosyl)-beta-D-galactopyranoside; octyl 3-deoxy-2-O-(6-deoxy-alpha-L-galactopyranosyl)-beta-D-xylo-hexopyranoside; N,N-Bis(4-Chlorobenzyl)-1h-1,2,3,4-Tetraazol-5-Amine; 2,4-Dinitrophenyl 2-Deoxy-2-Fluoro-Beta-D-Allopyranoside; N′-[(1E)-(3,5-dibromo-2,4-dihydroxyphenyl)methylidene]-4-methoxybenzohydrazide; N′-[(1E)-(3,5-dibromo-2,4-dihydroxyphenyl)methylidene]naphthalene-2-carbohydrazide; N-[(1R)-3-(4-HYDROXYPHENYL)-1-METHYLPROPYL]-2-(2-PHENYL-1H-INDOL-3-YL)ACETAMIDE; N-[(1R,2R,3E)-2-hydroxy-1-(hydroxymethyl)heptadec-3-en-1-yl]acetamide; N-[(1S)-1-(AMINOCARBONYL)-4-(ETHANIMIDOYLAMINO)BUTYL]BENZAMIDE; N-[(1S)-2-amino-1-phenylethyl]-5-(1H-pyrrolo[2,3-b]pyridin-4-yl)thiophene-2-carboxamide; N-[(1S)-2-methyl-1-(pyridin-4-ylcarbamoyl)propyl]cyclohexanecarboxamide; N-[(1S)-5-amino-1-(chloroacetyl)pentyl]-4-methylbenzenesulfonamide; N-[(2′,4′-DIFLUORO-4-HYDROXY-5-IODOBIPHENYL-3-YL)CARBONYL]-BETA-ALANINE; N-[(2R)-2-{[(2S)-2-(1,3-benzoxazol-2-yl)pyrrolidin-1-yl]carbonyl}hexyl]-N-hydroxyformamide; N-[(2R)-2-benzyl-4-(hydroxyamino)-4-oxobutanoyl]-L-alanine; N-[(2R)-5-(aminosulfonyl)-2,3-dihydro-1H-inden-2-yl]-2-propylpentanamide; N-[(3z)-5-Tert-Butyl-2-Phenyl-1,2-Dihydro-3h-Pyrazol-3-Ylidene]-N′-(4-Chlorophenyl)Urea; N-[(4-HYDROXY-8-IODOISOQUINOLIN-3-YL)CARBONYL]GLYCINE; N-[(6-BUTOXYNAPHTHALEN-2-YL)SULFONYL]-D-GLUTAMIC ACID; 2,6-anhydro-3-deoxy-3-fluoronononic acid; 4-Oxo-2-Phenylmethanesulfonyl-Octahydro-Pyrrolo[1,2-a]Pyrazine-6-Carboxylic Acid [1-(N-Hydroxycarbamimidoyl)-Piperidin-4-Ylmethyl]-Amide; 5-HYDROXY-2-(4-HYDROXYPHENYL)-1-BENZOFURAN-7-CARBONITRILE; 5-BROMO-2-{[(4-CHLOROPHENYL)SULFONYL]AMINO}BENZOIC ACID; 5,6,7,8-TETRAHYDRO[1]BENZOTHIENO[2,3-D]PYRIMIDIN-4(3H)-ONE; 4-{2-[(7-amino-2-furan-2-yl[1,2,4]triazolo[1,5-a][1,3,5]triazin-5-yl)amino]ethyl}phenol; 4-{2-[4-(2-Aminoethyl)Piperazin-1-Yl]Pyridin-4-Yl}-N-(3-Chloro-4-Methylphenyl)Pyrimidin-2-Amine; 4-{3-CHLORO-4-[3-(2,4-DICHLORO-BENZOYL)-UREIDO]-PHENOXY}-BUTYRIC ACID; 4-AMINO-2-HEXYLOXY-6-HYDROXYMETHYL-TETRAHYDRO-PYRAN-3,5-DIOL; 4-AMINO-2-OCTYLOXY-6-HYDROXYMETHYL-TETRAHYDRO-PYRAN-3,5-DIOL; 4-AMINO-2-TRIFLUOROMETHYL-5-HYDROXYMETHYLPYRIMIDINE PYROPHOSPHATE; 2,6-DIMETHYL-1-(3-[3-METHYL-5-ISOXAZOLYL]-PROPANYL)-4-[2-METHYL-4-ISOXAZOLYL]-PHENOL; 2,6-Dihydroanthra/1,9-Cd/Pyrazol-6-One; 4-Acetamido-2,4-Didexoy-D-Glycero-Beta-D-Galacto-Octopyranosylphosphonic Acid; 4-Acetyl-4-Guanidino-6-Methyl(Propyl)Carboxamide-4,5-Dihydro-2h-Pyran-2-Carboxylic Acid; 4-Amino-1-[(1s,3r,4r,7s)-7-Hydroxy-1-(Hydroxymethyl)-2,5-Dioxabicyclo[2.2.1]Hept-3-Yl]-5-Methylpyrimidin-2(1h)-One; 4-METHYL-N-METHYL-N- (2-PHENYL-2H-PYRAZOL-3-YL)BENZENESULFONAMIDE; 4-METHYL-PENTANOIC ACID {1-[4-GUANIDINO-1-(THIAZOLE-2-CARBONYL)-BUTYLCARBAMOYL]-2-METHYL-PROPYL}-AMIDE; 4-Morpholin-4-Yl-Piperidine-1-Carboxylic Acid [1-(3-Benzenesulfonyl-1-Propyl-Allylcarbamoyl)-2-Phenylethyl]-Amide; 2-[(1S)-1-BENZYL-2-SULFANYLETHYL]-1H-IMIDAZO[4,5-C]PYRIDIN-5-IUM; 4-(2-HYDROXYBENZYLAMINO)-N-(3-(4-FLUOROPHENOXY)PHENYL)PIPERIDINE-1-SULFONAMIDE; 2-[(2-chloro-4-iodophenyl)amino]-N-{[(2R)-2,3-dihydroxypropyl]oxy}-3,4-difluorobenzamide; 4-(6-{[(1R)-1-(hydroxymethyl)propyl]amino}imidazo[1,2-b]pyridazin-3-yl)benzoic acid; 4-(1,3-BENZODIOXOL-5-YLOXY)-2-[4-(1H-IMIDAZOL-1-YL)PHENOXY]-6-METHYLPYRIMIDINE; 2′,4′-Dinitrophenyl-2deoxy-2-Fluro-B-D-Cellobioside; 2-[(2-NAPHTHYLSULFONYL)AMINO]ETHYL DIHYDROGEN PHOSPHATE; 4-{2,6,8-Trioxo-9-[(2s,3r,4r)-2,3,4,5-Tetrahydroxypentyl]-1,2,3,6,8,9-Hexahydro-7h-Purin-7-Yl}Butyl Dihydrogen Phosphate; 4-(6-CYCLOHEXYLMETHOXY-9H-PURIN-2-YLAMINO)--BENZAMIDE; 2-[(4-fluorophenyl)sulfonylamino]-N-oxo-ethanamide; 4-(1,3-Benzodioxol-5-Yl)-5-(5-Ethyl-2,4-Dihydroxyphenyl)-2h-Pyrazole-3-Carboxylic Acid; 4-(2-Amino-Ethoxy)-2-[(3-Hydroxy-2-Methyl-5-Phosphonooxymethyl-Pyridin-4-Ylmethyl)-Amino]-but-3-Enoic Acid; 4-(6-{[(4-METHYLCYCLOHEXYL)AMINO]METHYL}-1,4-DIHYDROINDENO[1,2-C]PYRAZOL-3-YL)BENZOIC ACID; 4-(2-{[4{[3-(4-Chlorophenyl)Propyl]Sulfanyl}-6-(1-Piperazinyl)-1,3,5-Triazin-2-Yl]Amino}Ethyl)Phenol; 4-(2-aminoethoxy)-N-(2,5-diethoxyphenyl)-3,5-dimethylbenzamide; 4-(2-aminoethoxy)-N-(3-chloro-2-ethoxy-5-piperidin-1-ylphenyl)-3,5-dimethylbenzamide; 2-[(Formyl-Hydroxy-Amino)-Methyl]-Heptanoic Acid [1-(2-Hydroxymethyl-Pyrrolidine-1-Carbonyl)-2-Methyl-Propyl]-Amide; 4-HYDROXYBENZALDEHYDE O-(CYCLOHEXYLCARBONYL)OXIME; 4-METHYL-N-{(5E)-5-[(5-METHYL-2-FURYL)METHYLENE]-4-OXO-4,5-DIHYDRO-1,3-THIAZOL-2-YL}BENZENESULFONAMIDE; 2-[1-(4-CHLOROBENZOYL)-5-METHOXY-2-METHYL-1H-INDOL-3-YL]-N-[(1S)-1-(HYDROXYMETHYL)PROPYL]ACETAMIDE; 5-[2-CHLORO-4-(TRIFLUOROMETHYL)PHENOXY]-2-NITROBENZOIC ACID; 5,7-DIHYDROXY-2-(3,4,5-TRIHYDROXYPHENYL)-4H-CHROMEN-4-ONE; 2-[2-(2-Hydroxy-Ethoxy)-Ethoxy]-Ethanol; 5-HYDROXY-3-[(1R)-1-(1H-PYRROL-2-YL)ETHYL]-2H-INDOL-2-ONE; 5-hydroxy-4-(7-methoxy-1,1-dioxido-2H-1,2,4-benzothiadiazin-3-yl)-2-(3-methylbutyl)-6-phenylpyridazin-3(2H)-one; 5-AMINO-6-CYCLOHEXYL-4-HYDROXY-2-ISOBUTYL-HEXANOIC ACID; 2-[2-(4-CHLORO-PHENYLSULFANYL)-ACETYLAMINO]-3-(4-GUANIDINO-PHENYL)-PROPIONAMIDE; 5-Imino-4-(3-trifluoromethyl-phenylazo)-5H-pyrazol-3-ylamine; 5-IMINO-4-(2-TRIFLUOROMETHYL-PHENYLAZO)-5H-PYRAZOL-3-YLAMINE; 5-METHYL-3-(9-OXO-1,8-DIAZA-TRICYCLO[10.6.1.013,18]NONADECA-12(19),13,15,17-TETRAEN-10-YLCARBAMOYL)-HEXANOIC ACID; 74(Carboxy(4-Hydroxyphenyl)Acetyl)Amino)-7-Methoxy-(3-((1-Methyl-1h-Tetrazol-5-Yl)Thio)Methyl)-8-Oxo-5-Oxa-1-Azabicyclo[4.2.0]Oct-2-Ene-2-Carboxylic Acid; 6-methyl-5-[3-methyl-3-(3,4,5-trimethoxyphenyl)but-1-yn-1-yl]pyrimidine-2,4-diamine; 4-(1,3-BENZODIOXOL-5-YLOXY)-2-[4-(1H-IMIDAZOL-1-YL)PHENOXY]PYRIMIDINE; 7-(1,1-Dioxo-1h-Benzo[D]Isothiazol-3-Yloxymethyl)-2-(Oxalyl-Amino)-4,7-Dihydro-5h-Thieno[2,3-C]Pyran-3-Carboxylic Acid; 7,8-Dihydroxy-1-Methoxy-3-Methyl-10-Oxo-4,10-Dihydro-1h,3h-Pyrano[4,3-B]Chromene-9-Carboxylic Acid; 7-(1-Methyl-1,2,3-Triazol-4-Yl)-6-Formyl-2,7-Dihydro-[1,4]Thiazepine-3-Carboxylic Acid, Brl42715, C6-(N1-Methyl-1,2,3-Triazolylmethylene)Penem; 7-(2-Amino-2-Phenyl-Acetylamino)-3-Chloro-8-Oxo-1-Aza-Bicyclo[4.2.0]Oct-2-Ene-2-Carboxylic Acid; 7-(1-ETHYL-PROPYL)-7H-PYRROLO-[3,2-F]QUINAZOLINE-1,3-DIAMINE; 6-{4-[HYDROXY-(4-NITRO-PHENOXY)-PHOSPHORYL]-BUTYRYLAMINO}-HEXANOIC ACID; 7-(aminomethyl)-6-(2-chlorophenyl)-1-methyl-1H-benzimidazole-5-carbonitrile; 7,10,13-Tri(Carboxymethyl)-5,15-Dioxo-4,7,10,13,16-Pentaaza-1,19-Dithianonadecane; 6-CHLORO-4-(CYCLOHEXYLSULFINYL)-3-PROPYLQUINOLIN-2(1H)-ONE; 6-CYCLOHEXYLMETHOXY-2-(3-CHLOROANILINO) PURINE; 2-[4-[[(S)-1-[[(S)-2-[[(Rs)-3,3,3-Trifluoro-1-Isopropyl-2-Oxopropyl]Aminocarbonyl]Pyrrolidin-1-Yl-]Carbonyl]-2-Methylpropyl]Aminocarbonyl]Benzoylamino]Acetic Acid; 6-CYCLOHEXYLMETHYLOXY-5-NITROSO-PYRIMIDINE-2,4-DIAMINE; 6-ethyl-5-[(2S)-1-(3-methoxypropyl)-2-phenyl-1,2,3,4-tetrahydroquinolin-7-yl]pyrimidine-2,4-diamine; 6-ETHYL-5-[9-(3-METHOXYPROPYL)-9H-CARBAZOL-2-YL]PYRIMIDINE-2,4-DIAMINE; 6-Fluoro-2-(2′-Fluoro-1,1′-Biphenyl-4-Yl)-3-Methylquinoline-4-Carboxylic Acid; 7-(5-DEOXY-BETA-D-RIBOFURANOSYL)-5-IODO-7H-PYRROLO[2,3-D]PYRIMIDIN-4-AMINE; 2-[N′-(4-AMINO-BUTYL)-HYDRAZINOCARBONYL]-PYRROLIDINE-1-CARBOXYLIC ACID BENZYL ESTER; 6-amino-2-[(2-morpholin-4-ylethyl)amino]-3,7-dihydro-8H-Imidazo[4,5-g]quinazolin-8-one; 6-amino-2-[(thiophen-2-ylmethyl)amino]-1,7-dihydro-8H-Imidazo[4,5-g]quinazolin-8-one; 6-(Adenosine Tetraphosphate-Methyl)-7,8-Dihydropterin; 6-(cyclohexylamino)-9-[2-(4-methylpiperazin-1-yl)-ethyl]-9H-purine-2-carbonitrile; 2-[CARBOXY-(2-THIOPHEN-2-YL-ACETYLAMINO)-METHYL]-5-METHYLENE-5,6-DIHYDRO-2H-[1,3]THIAZINE-4-CARBOXYLIC ACID; 6-(cyclohexylsulfanyl)-1-(ethoxymethyl)-5-(1-methylethyl)pyrimidine-2,4(1H,3H)-dione; 2-{[(6-OXO-1,6-DIHYDROPYRIDIN-3-YL)METHYL]AMINO}-N-[4-PROPYL-3-(TRIFLUOROMETHYL)PHENYL]BENZAMIDE; 6-(Hydroxyethyldithio)-8-(Aminomethylthio)Octanoic Acid; 6-(N-Phenylcarbamyl)-2-Naphthalenecarboxamidine; 6-(Octahydro-1h-Indol-1-Ylmethyl)Decahydroquinazoline-2,4-Diamine; 2-{[N-(2-ACETYL-5-CHLORO-4-FLUOROPHENYL)GLYCYL]AMINO}BENZOIC ACID; 4-[(1S)-1-(3-fluoro-4-methoxyphenyl)-2-(2-methoxy-5-nitrophenyl)ethyl]-1H-imidazol-2-amine; 4-[(1S,2R,5S)-4,4,8-TRIMETHYL-3-OXABICYCLO[3.3.1]NON-7-EN-2-YL]PHENOL; 2-{2-[(3,5-dimethylphenyl)amino]pyrimidin-4-yl}-N-[(1S)-2-hydroxy-1-methylethyl]-4-methyl-1,3-thiazole-5-carboxamide; 4-[(3S)-1-AZABICYCLO[2.2.2]OCT-3-YLAMINO]-3-(1H-BENZIMIDAZOL-2-YL)-6-CHLOROQUINOLIN-2(1H)-ONE; 4-[(4-bromo-2-{[(3R,5S)-3,5-dimethylpiperidin-1-yl]carbonyl}phenyl)amino]-4-oxobutanoic acid; 4-[(4-Imidazo[1,2-a]Pyridin-3-Ylpyrimidin-2-Yl)Amino]Benzenesulfonamide; 4,6-O(1-CARBOXYETHYLIDENE)-BETA-D-MANNOSE-(1-&gt;4)-BETA-D-GLUCURONIC ACID; 4,6-O-(1-CARBOXYETHYLIDENE)-BETA-D-MANNOSE-(1->4)-BETA-D-GLUCURONIC ACID; 2-{4-[(3,5-DIMETHYLANILINO)-CARBONYL-METHYL]-PHENOXY}-2-METHYLPROPIONIC ACID; 3-methyl-N4pyridin-4-ylmethyl)imidazo[1,2-a]pyrazin-8-amine; 3-Nitro-4-(2-Oxo-Pyrrolidin-1-Yl)-Benzenesulfonamide; 2-{4-[4-({4-[2-methyl-1-(1-methylethyl)-1H-imidazol-5-yl]pyrimidin-2-yl}amino)phenyl]piperazin-1-yl}-2-oxoethanol; 3-OXO-N-[(3S)-2-OXOPYRROLIDIN-3-YL]DODECANAMIDE; 3-OXO-OCTANOIC ACID (2-OXO-TETRAHYDRO-FURAN-3-YL)-AMIDE; 3′-Phosphate-Adenosine-5′-Diphosphate; 2-{HYDROXY[2-NITRO-4-(TRIFLUOROMETHYL)PHENYL]METHYLENE}CYCLOHEXANE-1,3-DIONE; 2-{N′-[2(5-Amino-1-Phenylcarbamoyl-Pentylcarbamoyl)-Hexyl]-Hydrazinomethyl}-Hexanoic Acid(5-Amino-1-Phenylcarbamoyl-Pentyl)-Amide; 3-Pyridin-4-Yl-2,4-Dihydro-Indeno[1,2-.C.]Pyrazole; 2-{4-[5-(4-chlorophenyl)-4-pyrimidin-4-yl-1H-pyrazol-3-yl]piperidin-1-yl}-2-oxoethanol; 2-ETHOXYETHYL (2S,3S)-4-((S)-2-BENZYL-3-OXO-4-((3AR,8R,8AS)-2-OXO-3,3A,8,8A-TETRAHYDRO-2H-INDENO[1,2-D]OXAZOL-8-YL)-2,3-DIHYDRO-1H-PYRROL-2-YL)-3-HYDROXY-1-PHENYLBUTAN-2-YLCARBAMATE; 4-(2,4-Dimethyl-Thiazol-5-Yl)-Pyrimidin-2-Ylamine; 2-CHLORO-4-[(7R,7AS)-7-HYDROXY-1,3-DIOXOTETRAHYDRO-1H-PYRROLO[1,2-C]IMIDAZOL-2(3H)-YL]-3-METHYLBENZONITRILE; 4-(2,4-dichlorophenyl)-5-phenyldiazenyl-pyrimidin-2-amine; 2-Amino-3-[4-Hydroxy-6-Oxo-3-(2-Phenyl-Cyclopropylimino)-Cyclohexa-1,4-Dienyl]-Propionic Acid; 4-(1-Benzyl-3-Carbamoylmethyl-2-Methyl-1h-Indol-5-Yloxy)-Butyric Acid; 4-(2,5-DIAMINO-5-HYDROXY-PENTYL)-PHENOL; 2-Amino-3(3-Hydroxy-7,8-Dihydro-6h-Cyclohepta[D]-4-Isoxazolyl)Propionic Acid; 4-(2-amino-1,3-thiazol-4-yl)pyrimidin-2-amine; 4-(2-AMINOETHYL)BENZENESULFONAMIDE; 4-(2-amino-1-methyl-1H-imidazo[4,5-b]pyridin-6-yl)phenol; 4-(2-AMINOETHYL)BENZENESULFONYL FLUORIDE; 4-(2-AMINOPHENYL)-4-OXOBUTANOIC ACID; 4-(2-HYDROXYPHENYLSULFINYL)-BUTYLPHOSPHONIC ACID; 4-(2-HYDROXYPHENYLTHIO)-1-BUTENYLPHOSPHONIC ACID; 4-(2-methoxyethoxy)-6-methylpyrimidin-2-amine; 4-(2-METHOXYPHENYL)-2-OXOBUT-3-ENOIC ACID; 2-amino-4-[2,4-dichloro-5-(2-pyrrolidin-1-ylethoxy)phenyl]-N-ethylthieno[2,3-d]pyrimidine-6-carboxamide; 4-(2-Thienyl)-1-(4-Methylbenzyl)-1h-Imidazole; 4-(3-ethylthiophen-2-yl)benzene-1,2-diol; 4-(3-amino-1H-indazol-5-yl)-N-tert-butylbenzenesulfonamide; 4-(3-Pyridin-2-Yl-1h-Pyrazol-4-Yl)Quinoline; 4-(4-chlorobenzyl)-1-(7H-pyrrolo[2,3-d]pyrimidin-4-yl)piperidin-4-aminium; 4-(4-FLUOROPHENYL)-1-CYCLOROPROPYLMETHYL-5-(4-PYRIDYL)-IMIDAZOLE; 4-(5-Bromo-2-Oxo-2h-Indol-3-Ylazo)-Benzenesulfonamide; 4-(4-Hydroxy-3-Isopropylphenylthio)-2-Isopropylphenol; 4-(4-hydroxy-3-methylphenyl)-6-phenylpyrimidin-2(5H)-one; 4-(4-HYDROXYPHENYL)-1-NAPHTHALDEHYDE OXIME; 4-(4-methoxy-1H-pyrrolo[2,3-b]pyridin-3-yl)pyrimidin-2-amine; 4-(4-methyl-1,3-dioxo-1,3-dihydro-2H-isoindol-2-yl)benzonitrile; 4-(4-propoxy-1H-pyrrolo[2,3-b]pyridin-3-yl)pyrimidin-2-amine; 4-(4-STYRYL-PHENYLCARBAMOYL)-BUTYRIC ACID; 4-(4-fluoro-phenylazo)-5-imino-5H-pyrazol-3-ylamine; 4-(5-phenyl-1H-pyrrolo[2,3-b]pyridin-3-yl)benzoic acid; 4-(6-HYDROXY-1H-INDAZOL-3-YL)BENZENE-1,3-DIOL; 2-Amino-5-Bromo-6-Phenylpyrimidin-4-Ol; 4-(Acetylamino)-3-[(Aminoacetyl)Amino]Benzoic Acid; 2-butoxy-9-(2,6-difluorobenzyl)-N-(2-morpholin-4-ylethyl)-9H-purin-6-amine; 4-(Acetylamino)-3-Amino Benzoic Acid; 4-(Acetylamino)-3-Guanidinobenzoic Acid; 4-(ACETYLAMINO)-3-HYDROXY-5-NITROBENZOIC ACID; 4-(ACETYLAMINO)-5-AMINO-3-HYDROXYBENZOIC ACID; 4-(acetylamino)-N-(4-fluorophenyl)-1H-pyrazole-3-carboxamide; 4-(Aminosulfonyl)-N-[(2,3,4-Trifluorophenyl)Methyl]-Benzamide; 4-(Aminosulfonyl)-N-[(2,4,6-Trifluorophenyl)Methyl]-Benzamide; 2-CHLORO-N-(3-CYANO-5,6-DIHYDRO-4H-CYCLOPENTA[B]THIOPHEN-2-YL)-5-DIETHYLSULFAMOYL-BENZAMIDE; 4-(Aminosulfonyl)-N-[(2,5-Difluorophenyl)Methyl]-Benzamide; 4-(Aminosulfonyl)-N-[(3,4,5-Trifluorophenyl)Methyl]-Benzamide; 2-CHLORO-N-[(3R)-2-OXO-1,2,3,4-TETRAHYDROQUINOLIN-3-YL]-6H-THIENO[2,3-B]PYRROLE-5-CARBOXAMIDE; 4-(BENZHYDRYLOXY)-1-[3-(1H-TETRAAZOL-5-YL)PROPYL]PIPERIDINE; 4-(dihydroxyboranyl)-2-({[4-(phenylsulfonyl)thiophen-2-yl]sulfonyl}amino)benzoic acid; 4-(N-ACETYLAMINO)-3-[N-(2-ETHYLBUTANOYLAMINO)]BENZOIC ACID; 4-(Fluorophenyl)-1-Cyclopropylmethyl-5-(2-Amino-4-Pyrimidinyl)Imidazole; 4-(DIMETHYLAMINO)BUTYL IMIDOTHIOCARBAMATE; 4-(METHYLSULFONYL)BENZENECARBOXIMIDAMIDE; 4-(N,N-Dimethylamino)Cinnamoyl-Coa; 4-(Methylsulfanyl)-2-Oxobutanoic Acid; 4-(quinolin-3-ylmethyl)piperidine-1-carboxylic acid; 4,4′[1,6-Hexanediylbis(Oxy)]Bisbenzenecarboximidamide; 4,4′-BIS([H]METHYLSULFONYL)-2,2′,5,5′-TETRACHLOROBIPHENYL; 4,5,6,7-Tetrachloro-3h-Isobenzofuran-1-One; 4,5-bis(4-methoxyphenyl)-2-thiophen-2-yl-1H-imidazole; 4,5-Dihydroxy-Tetrahydro-Pyran-2-Carboxylic Acid; 4,5-Dimethyl-1,2-Phenylenediamine; 4,6-O-(1-Carboxyethylidene)-Beta-D-Glucose; 4-[(5-bromopyridin-2-yl)amino]-4-oxobutanoic acid; 2-hydroxy-7-methoxy-5-methyl-naphthalene-1-carboxylic acid meso-2,5-dihydroxy-cyclopent-3-enyl ester; 4-[(5-methoxy-2-methylphenoxy)methyl]pyridine; 4-[(6-Amino-4-Pyrimidinyl)Amino]Benzenesulfonamide; 4-[(6-chloropyrazin-2-yl)amino]benzenesulfonamide; 2-HYDROXY-1,1′-BIPHENYL-2-SULFINIC ACID; 4-[(E)-(3,5-DIAMINO-1H-PYRAZOL-4-YL)DIAZENYL]PHENOL; 2-Fluoro-2-Deoxy-Beta-D-Galactopyranosyl-Beta-D-Glucopyranose; 4-[1-allyl-7-(trifluoromethyl)-1H-indazol-3-yl]benzene-1,3-diol; 4-[(METHYLSULFONYL)AMINO]BENZOIC ACID; 4-[3-(1,4-diazepan-1-ylcarbonyl)-4-fluorobenzyl]phthalazin-1(2H)-one; 4-[3-(1H-BENZIMIDAZOL-2-YL)-1H-INDAZOL-6-YL]-2-METHOXYPHENOL; 4-[3-(2-Chloro-4,5-difluoro-benzoyl)ureido]-3-trifluoromethoxybenzoic acid; 4-[3-(3-NITROPHENYL)-1,2,4-OXADIAZOL-5-YL]BUTANOIC ACID; 4-[3-(4-CHLOROPHENYL)-1H-PYRAZOL-5-YL]PIPERIDINE; 4-[3-(4-chlorophenyl)-2,1-benzisoxazol-5-yl]pyrimidin-2-amine; 4-[3-(4-FLUOROPHENYL)-1H-PYRAZOL-4-YL]PYRIDINE; 4-[3-(dibenzylamino)phenyl]-2,4-dioxobutanoic acid; 4-[5-Pyridin-4-Yl-1h-[1,2,4]Triazol-3-Yl]-Pyridine-2-Carbonitrile; 2-IMINO-5-(1-PYRIDIN-2-YL-METH-(E)-YLIDENE)-1,3-THIAZOLIDIN-4-ONE; 4-[4-(4-methoxyphenyl)-5-methyl-1H-pyrazol-3-yl]benzene-1,3-diol; 3-({1-[3-CARBAMIMIDOYL-1-(4-CARBAMIMIDOYL-BENZYLCARBAMOYL)-PROPYLCARBAMOYL]-2-METHYL-BUTYLSULFAMOYL}-METHYL)-BENZOIC ACID; 4-[3-Hydroxyanilino]-6,7-Dimethoxyquinazoline; 4-[8-(3-nitrophenyl)-1,7-naphthyridin-6-yl]benzoic acid; 2-METHYL-FURAN-3-CARBOTHIOIC ACID [4-CHLORO-3-(3-METHYL-BUT-2-ENYLOXY)-PHENYL]-AMIDE; 4-{[(2R)-2-(2-methylphenyl)pyrrolidin-1-yl]carbonyl}benzene-1,3-diol; 4-{[(2S)-3-(tert-butylamino)-2-hydroxypropyl]oxy}-3H-indole-2-carbonitrile; 2′-Monophosphoadenosine-5′-Diphosphate; 4-Hydroxy-3-[(1s)-3-Oxo-1-Phenylbutyl]-2h-Chromen-2-One; 4-chloro-6-{5-[(2-morpholin-4-ylethyl)amino]-1,2-benzisoxazol-3-yl}benzene-1,3-diol; 2′-Monophosphoadenosine-5-Diphosphoribose; 2-O-(4′-AMIDINOPHENYL)-5-O-(3″-AMIDINOPHENYL)-1,4:3,6-DIANHYDRO-D-SORBITOL; dichlorophenyl)-N-(pyridin-4-ylmethyl)pyrazolo[1,5-a]pyrimidin-7-amine; 4-{2,4-Bis[(3-Nitrobenzoyl)Amino]Phenoxy}Phthalic Acid; 4-{2-[(3-Nitrobenzoyl)Amino]Phenoxy}Phthalic Acid; 4-Amino-2-Deoxy-2,3-Dehydro-N-Neuraminic Acid; 4-bromo-2-{[(3R,5S)-3,5-dimethylpiperidin-1-yl]carbonyl}aniline; 4-amino-7,7-dimethyl-7,8-dihydroquinazolin-5(6H)-one; 4-amino-N-[4-(benzyloxy)phenyl]butanamide; 4-bromo-2-{[(2R)-2-(2-chlorobenzyl)pyrrolidin-1-yl]carbonyl}aniline; 4-Amino-5-Hydroxymethyl-2-Methylpyrimidine; 4-Bromo-3-Hydroxy-3-Methyl Butyl Diphosphate; 4-bromo-6-(6-hydroxy-1,2-benzisoxazol-3-yl)benzene-1,3-diol; 2-PHENYLAMINO-4-METHYL-5-ACETYL THIAZOLE; 4-CHLORO-3′,3″-DIBROMOPHENOL-1,8-NAPHTHALEIN; 3-({4-[(5-CHLORO-1,3-BENZODIOXOL-4-YL)AMINO]PYRIMIDIN-2-YL}AMINO)BENZAMIDE; 4-[4-(4-Methyl-2-Methylamino-Thiazol-5-Yl)-Pyrimidin-2-Ylamino]-Phenol; 3-({2-[(4-{[6-(CYCLOHEXYLMETHOXY)-9H-PURIN-2-YL]AMINO}PHENYL)SULFONYL]ETHYL}AMINO)PROPAN-1-OL; 4-Epi-Vancosaminyl Derivative of Vancomycin; 3-(2-CHLOROPHENYL)-1-(2-{[(1S)-2-HYDROXY-1,2-DIMETHYLPROPYL]AMINO}PYRIMIDIN-4-YL)-1-(4-METHOXYPHENYL)UREA; 4′-FLUORO-1,1′-BIPHENYL-4-CARBOXYLIC ACID; 3-({4-[(6-CHLORO-1-BENZOTHIEN-2-YL)SULFONYL]-2-OXOPIPERAZIN-1-YL}METHYL)BENZENECARBOXIMIDAMIDE; 4-Diphosphocytidyl-2-C-Methyl-D-Erythritol 2-Phosphate; 4-Hydroxy-3,4-Dihydro-1h-Pyrimidin-2-One; 3-(1,1-dioxido-4H-1,2,4-benzothiadiazin-3-yl)-4-hydroxy-1-(3-methylbutyl)quinolin-2(1H)-one; 4-Hydroxy-L-Threonine-5-Monophosphate; 3-(2-AMINO-6-BENZOYLQUINAZOLIN-3(4H)-YL)-N-CYCLOHEXYL-N-METHYLPROPANAMIDE; 4-HYDROXY-N-PROPARGYL-1(R)-AMINOINDAN; 3-(1h-Indol-3-Yl)-2-[4-(4-Phenyl-Piperidin-1-Yl)-Benzenesulfonylamino]-Propionic Acid; 4-Imino-5-Methidyl-2-Methylpyrimidine; 4-Imino-5-Methidyl-2-Trifluoromethylpyrimidine; 3-(1H-INDOL-3-YL)-4-{1-[2-(1-METHYLPYRROLIDIN-2-YL)ETHYL]-1H-INDOL-3-YL}-1H-PYRROLE-2,5-DIONE; 4-Iodobenzo[B]Thiophene-2-Carboxamidine; 4-Methyl-5-Hydroxyethylthiazole; 4-methyl-7,8-dihydro-5H-thiopyrano[4,3-d]pyrimidin-2-amine; 4-Methyl-5-Hydroxyethylthiazole Phosphate; 4-NITRO-BENZYLPHOSPHONOBUTANOYL-GLYCINE; 3-(2-aminoquinazolin-6-yl)-4-methyl-1-[3-(trifluoromethyl)phenyl]pyridin-2(1H)-one; 4-Sulfonamide-[1-(4-Aminobutane)]Benzamide; 4-Sulfonamide-[4-(Thiomethylaminobutane)]Benzamide; 4-TERT-BUTYLBENZENESULFONIC ACID; 5-(1,4-DIAZEPAN-1-SULFONYL)ISOQUINOLINE; 5-(2,5-DICHLOROPHENYL)-2-FUROIC ACID; 3-(2-AMINOQUINAZOLIN-6-YL)-4-METHYL-N-[3-(TRIFLUOROMETHYL)PHENYL]BENZAMIDE; 5-(dodecylthio)-1H-1,2,3-triazole-4-carboxylic acid; 5-(3-Amino-4,4-Dihyroxy-Butylsulfanylmethyl)-Tetrahydro-Furan-2,3,4-Triol; 5-(3-carbamoylbenzyl)-5,6,7,8,9,10-hexahydrocyclohepta[b]indole-4-carboxylic acid; 5-(3-HYDROXYPHENYL)ISOTHIAZOL-3(2H)-ONE 1,1-DIOXIDE; 3-(3,5-Dibromo-4-Hydroxy-Benzoyl)-2-Ethyl-Benzofuran-6-Sulfonic Acid (4-Sulfamoyl-Phenyl)-Amide; 5-(4-Morpholin-4-Yl-Phenylsulfanyl)-2,4-Quinazolinediamine; 3-(3-methoxybenzyl)-1H-pyrrolo[2,3-b]pyridine; 5-(5-(4-(5-hydro-4-methyl-2-oxazolyl)phenoxy)pentyl)-3-methyl isoxazole; 3-(3,5-Dibromo-4-Hydroxy-Benzoyl)-2-Ethyl-Benzofuran-6-Sulfonic Acid [4-(Thiazol-2-Ylsulfamoyl)-Phenyl]-Amide; 5-(6-D-Ribitylamino-2,4-Dihydroxypyrimidin-5-Yl)-1-Pentyl-Phosphonic Acid; 5-(7-(4-(4,5-dihydro-2-oxazolyl)phenoxy)heptyl)-3-methyl isoxazole; 5-(7-(5-hydro-4-ethyl-2-oxazolyl)phenoxy)heptyl)-3-methyl isoxazole; 5-(7-(5-hydro-4-methyl-2-oxazolyl)phenoxy)heptyl)-3-methyl isoxazole; 5-(7-(6-chloro-4-(5-hydro-4-methyl-2-oxazolyl)phenoxy)heptyl)-3-methyl isoxazole; 5-(AMINOCARBONYL)-1,1′:1,1″-TERPHENYL-3-CARBOXYLICACID; 5,10-Dimethylene Tetrahydromethanopterin; 5(R)-5-Fluoro-Beta-D-Xylopyranosyl-Enzyme Intermediate; 5,10,15,20-Tetrakis(4-Sulpfonatophenyl)-21h,23h-Porphine; 5-(3,3-Dihydroxypropeny)-3-Methoxy-Benzene-1,2-Diol; 5-(Aminomethyl)-2-Methylpyrimidin-4-Amine; 5-(Aminomethyl)-6-(2,4-Dichlorophenyl)-2-(3,5-Dimethoxyphenyl)Pyrimidin-4-Amine; 3-(5-{[4-(AMINOMETHYL)PIPERIDIN-1-YL]METHYL}-1H-INDOL-2-YL)QUINOLIN-2(1H)-ONE; 5-(DIMETHYLAMINO)-2-NAPHTHALENESULFONIC ACID; 5-(HEXAHYDRO-2-OXO-1H-THIENO[3,4-D]IMIDAZOL-6-YL)PENTANAL; 5-(Hydroxy-Methyl-Amino)-3-Methyl-Pyrrolidine-2-Carboxylic Acid; 3-(5-Amino-7-Hydroxy-[1,2,3]Triazolo[4,5-D]Pyrimidin-2-Yl)-N-[2-(2-(Hydroxymethyl-Phenylsulfanyl)-Benzyl]-Benzamide; 5,5-dimethyl-2-morpholin-4-yl-5,6-dihydro-1,3-benzothiazol-7(4H)-one; 5,6-dichloro-1-beta-D-ribofuranosyl-1H-benzimidazole; 5-[(5-fluoro-3-methyl-1H-indazol-4-yl)oxy]benzene-1,3-dicarbonitrile; 3-(7-DIAMINOMETHYL-NAPHTHALEN-2-YL)-PROPIONIC ACID ETHYL ESTER; 5-[(1S)-1-(3-chlorophenyl)ethoxy]quinazoline-2,4-diamine; 5-[(4-Methylphenyl)Sulfanyl]-2,4-Quinazolinediamine; 5,6-Dihydro-Benzo[H]Cinnolin-3-Ylamine; 5-(DIMETHYLAMINO)-1-NAPHTHALENESULFONIC ACID(DANSYL ACID); 5′-[[2-(Aminooxy)Ethyl]Methylsulfonio]-5-Deoxy-Adenosine; 5-[2-(4-hydroxyphenyl)ethyl]benzene-1,3-diol; 5-[2-(TRIFLUOROMETHOXY)PHENYL]-2-FUROIC ACID; 5-[2-(TRIFLUOROMETHYL)PHENYL]-2-FUROIC ACID; 3-(9-HYDROXY-1,3-DIOXO-4-PHENYL-2,3-DIHYDROPYRROLO[3,4-C]CARBAZOL-6(1H)-YL)PROPANOIC ACID; 3(R)-METHYLCARBAMOYL-7-SULFOAMINO-3,4-DIHYDRO-1H-ISOQUINOLINE-2-CARBOXYLIC ACID TERT-BUTYL ESTER; 5′-{[4-(aminooxy)butyl](methyl)amino}-5′-deoxy-8-ethenyladenosine; 3,6-DIAMINO-5-CYANO-4-(4-ETHOXYPHENYL)THIENO[2,3-B]PYRIDINE-2-CARBOXAMIDE; 5-amino-2,4,6-tribromobenzene-1,3-dicarboxylic acid; 5-[4-Tert-Butylphenylsulfanyl]-2,4-Quinazolinediamine; 5-{[(2-Amino-9h-Purin-6-Yl)Oxy]Methyl}-2-Pyrrolidinone; 3-[[(METHYLAMINO)SULFONYL]AMINO]-2-OXO-6-PHENYL-N-[3,3,3-TRIFLUORO-1-(1-METHYLETHYL)-2-OXOPHENYL]-1(2H)-PYRIDINE ACETAMIDE; 5-ALPHA-PREGNANE-3-BETA-OL-HEMISUCCINATE; 5-amino-1-(4-chlorophenyl)-1H-pyrazole-4-carbonitrile; 5-[3-(2,5-dimethoxyphenyl)prop-1-yn-1-yl]-6-ethylpyrimidine-2,4-diamine; 5-amino-2-methyl-N-[(1R)-1-naphthalen-1-ylethyl]benzamide; 3,5-Dimethyl-1h-Pyrazole-4-Carboxylic Acid Ethyl Ester; 5-Amino-5-Deoxy-Cellobiono-1,5-Lactam; 5-Aminocarbonyl-3-Nitrophenyl-Alpha-D-Galactopyranose; 5-AMINO-NAPHTALENE-2-MONOSULFONATE; 5-Beta-D-Ribofuranosylnicotinamide Adenine Dinucleotide; 5-HEPTYL-6-HYDROXY-1,3-BENZOTHIAZOLE-4,7-DIONE; 3,8,9,10-Tetrahydroxy-7-Hydroxymethyl-6-Oxa-1,3-Diaza-Spiro[4.5]Decane-2,4-Dione; 5-bromo-3-(pyrrolidin-1-ylsulfonyl)-1H-indole-2-carboxamide; 5-Guanosine-Diphosphate-Monothiophosphate; 3-[(1E,7E)-8-(2,6-dioxo-1,2,3,6-tetrahydropyrimidin-4-yl)-3,6-dioxa-2,7-diazaocta-1,7-dien-1-yl]benzoic acid; 3-[({(1E)-[2-(trifluoromethyl)phenyl]methylidene}amino)oxy]propanoic acid; 3-[(1-Amino-2-Carboxy-Ethyl)-Hydroxy-Phosphinoyl]-2-Methyl-Propionic Acid; 5-Hydroxymethyluridine-2′-Deoxy-5′-Monophosphate; 5-hydroxynaphthalene-1-sulfonamide; 5-Hydroxy-N-Propargyl-1(R)-Aminoindan; 5-Iodo-2′-Deoxyuridine-5-Monophosphate; 3-[(1R)-1-(2,6-dichloro-3-fluorophenyl)ethoxy]-5-(1-piperidin-4-yl-1H-pyrazol-4-yl)pyridin-2-amine; 5-Mercaptoethanol-2-Decenoyl-Coenzyme A; 5-Methoxy-1,2-Dimethyl-3-(4-Nitrophenoxymethyl)Indole-4,7-Dione; 3-[(4-CHLOROANILINO)SULFONYL]THIOPHENE-2-CARBOXYLIC ACID; 3-[(2,4-Dichlorobenzoyl)(Isopropyl)Amino]-5-Phenylthiophene-2-Carboxylic Acid; 5-METHYL-2-[(PHENYLSULFONYL)AMINO]BENZOIC ACID; 5-Methyl-2′-Deoxypseudouridine; 5-Methyl-5-(4-Phenoxy-Phenyl)-Pyrimidine-2,4,6-Trione; 5-Methyl-5,6,7,8-Tetrahydrofolic Acid; 3-[(4-fluorophenyl)sulfanyl]-N-(4-methyl-1,3-thiazol-2-yl)-6-[(4-methyl-4H-1,2,4-triazol-3-yl)sulfanyl]pyridine-2-carboxamide; 5--Monophosphate-9-Beta-D-Ribofuranosyl Xanthine; 5-methyl-N-[4-(trifluoromethyl)phenyl][1,2,4]triazolo[1,5-a]pyrimidin-7-amine; 5-N-Acetyl-4-Amino-6-Diethylcarboxamide-4,5-Dihydro-2h-Pyran-2-Carboxylic Acid; 5-QUINOXALIN-6-YLMETHYLENE-THIAZOLIDINE-2,4-DIONE; 5-N-Acetyl-3-(1-Ethylpropyl)-1-Cyclohexene-1-Carboxylic Acid; 3-[3-chloro-5-(5-{[(1S)-1-phenylethyl]amino}isoxazolo[5,4-c]pyridin-3-yl)phenyl]propan-1-ol; N-[(BENZYLOXY)CARBONYL]-L-CYSTEINYLGLYCINE; 6-(3-METHYL-1,4-DIOXO-1,4-DIHYDRONAPHTHALEN-2-YL)HEXANOIC ACID; 6-(2-fluorobenzyl)-2,4-dimethyl-4,6-dihydro-5H-thieno[2′,3′:4,5]pyrrolo[2,3-d]pyridazin-5-one; 6-(2-fluorophenyl)-N-(pyridin-3-ylmethyl)imidazo[1,2-a]pyrazin-8-amine; 6-(2-HYDROXY-CYCLOPENTYL)-7-OXO-HEPTANAMIDINE; 6-(2-Oxo-Hexahydro-Thieno[3,4-D]Imidazol-4-Yl)-Hexanoic Acid; 6-(2,5-Dimethoxy-Benzyl)-5-Methyl-Pyrido[2,3-D]Pyrimidine-2,4-Diamine; 6,7,12,13-tetrahydro-5H-indolo[2,3-a]pyrrolo[3,4-c]carbazol-5-one; 6-(3-BROMO-2-NAPHTHYL)-1,3,5-TRIAZINE-2,4-DIAMINE; 3-{[(2,2,5,5-TETRAMETHYL-1-OXO-4-PHENYL-2,5-DIHYDRO-1H-PYRROLIUM-3-YL)METHYL]DISULFANYL}-D-ALANINE; 5-Nitroso-6-Ribityl-Amino-2,4(1h,3h)-Pyrimidinedione; 5-n-undecyl-6-hydroxy-4,7-dioxobenzothiazole; ‘5’-O-(N-(L-Alanyl)-Sulfamoyl)Adenosine; 3-[6-bromo-2-fluoro-3-(1H-pyrazolo[3,4-c]pyridazin-3-ylmethyl)phenoxy]-5-chlorobenzonitrile; ‘5’-O-(N-(L-Prolyl)-Sulfamoyl)Adenosine; 3-[Isopropyl(4-Methylbenzoyl)Amino]-5-Phenylthiophene-2-Carboxylic Acid; 5′-O-(N-(L-Threonyl)-Sulfamoyl)Adenosine; 3-[N-[Benzyloxycarbonyl]-Phenylalaninyl-Amino]-5-Phenyl-Pentane-1-Sulfonylmethylbenzene; 3-{[(1R)-1-phenylethyl]amino}-4-(pyridin-4-ylamino)cyclobut-3-ene-1,2-dione; 5-Phospho-D-Arabinohydroxamic Acid; 5-Phosphoribosyl-1-(Beta-Methylene) Pyrophosphate; 3-{[4-([AMINO(IMINO)METHYL]AMINOSULFONYL)ANILINO]METHYLENE}-2-OXO-2,3-DIHYDRO-1H-INDOLE; 3-{2,6,8-trioxo-9-[(2R,3R,4R)-2,3,4,5-tetrahydroxypentyl]-1,2,3,6,8,9-hexahydro-7H-purin-7-Yl}propyl dihydrogen phosphate; 6-(2,6-dibromophenyl)pyrido[2,3-d]pyrimidine-2,7-diamine; 3-{[(9-CYANO-9,10-DIHYDRO-10-METHYLACRIDIN-9-YL)CARBONYL]AMINO}PROPANOIC ACID; 6-(Oxalyl-Amino)-1h-Indole-5-Carboxylic Acid; 6,4′-Dihydroxy-3-Methyl-3′,5′-Dibromoflavone; 6,7-Dioxo-5h-8-Ribitylaminolumazine; 3-{[(4-methylphenyl)sulfonyl]amino}propyl pyridin-4-ylcarbamate; 6-[2-(1H-INDOL-6-YL)ETHYL]PYRIDIN-2-AMINE; 3-{2,6,8-trioxo-9-[(2S,3R,4R)-2,3,4,5-tetrahydroxypentyl]-1,2,3,6,8,9-hexahydro-7H-purin-7-Yl}propyl dihydrogen phosphate; 6-[4-(2-fluorophenyl)-1,3-oxazol-5-yl]-N-(1-methylethyl)-1,3-benzothiazol-2-amine; 3-{6-[(8-HYDROXY-QUINOLINE-2-CARBONYL)-AMINO]-2-THIOPHEN-2-YL-HEXANOYLAMINO}-4-OXO-BUTYRI ACID; 6-[5-(2-Oxo-Hexahydro-Thieno[3,4-D]Imidazol-4-Yl)-Pentanoylamino]-Hexanoic Acid; 6-[BIS(2,2,2-TRIFLUOROETHYL)AMINO]-4-(TRIFLUOROMETHYL)QUINOLIN-2(1H)-ONE; 6-[N-(1-Isopropyl-1,2,3,4-Tetrahydro-7-Isoquinolinyl)Carbamyl]-2-Naphthalenecarboxamidine; 6-[N-(1-Isopropyl-3,4-Dihydro-7-Isoquinolinyl)Carbamyl]-2-Naphthalenecarboxamidine; 6-[N-(4-(Aminomethyl)Phenyl)Carbamyl]-2-Naphthalenecarboxamidine; 6-[N-(4-Ethyl-1,2,3,4-Tetrahydro-6-Isoquinolinyl)Carbamyl]-2-Naphthalenecarboxamidine; 6-{[(CYCLOHEXYLAMINO)CARBONYL]AMINO}HEXANOIC ACID; 6-{[6-(1-methyl-1H-pyrazol-4-yl)[1,2,4]triazolo[4,3-b]pyridazin-3-yl]sulfanyl}quinoline; 6-{4-[4-(4-CHLOROPHENYL)PIPERIDIN-4-YL]PHENYL}-9H-PURINE; 6-amino-2-methyl-1,7-dihydro-8H-imidazo[4,5-g]quinazolin-8-one; 6-AMINO-3,7-DIHYDRO-IMIDAZO[4,5-G]QUINAZOLIN-8-ONE; 6-amino-4-(2-phenylethyl)-1,7-dihydro-8H-imidazo[4,5-g]quinazolin-8-one; 6-Amino-4-Hydroxymethyl-Cyclohex-4-Ene-1,2,3-Triol; 6-AMINO-BENZO[DE]ISOQUINOLINE-1,3-DIONE; 6-BENZYL-1-BENZYLOXYMETHYL-5-ISOPROPYL URACIL; 3-AMINO-3-BENZYL-9-CARBOXAMIDE[4.3.0]BICYCLO-1,6-DIAZANONAN-2-ONE; 6-Chloro-2-(2-Hydroxy-Biphenyl-3-Yl)-1h-Indole-5-Carboxamidine; 6-Hydro-1-Methyladenosine-5′-Monophosphate; 3-Aminomethyl-Pyridinium-Adenine-Dinucleotide; 6-Hydroxy-1,6-Dihydro Purine Nucleoside; 6-ETHYL-5-PHENYLPYRIMIDINE-2,4-DIAMINE; 6-Deoxy-2-O-Methyl-Alpha-L-Galactopyranose; 6-Hydroxy-6-Methyl-Heptan-3-One; 3-BENZYLOXYCARBONYLAMINO-2-HYDROXY-4-PHENYL-BUTYRIC ACID; 3-bromo-5-phenyl-N-(pyridin-3-ylmethyl)pyrazolo[1,5-a]pyrimidin-7-amine; 3-bromo-6-phenyl-N-(pyrimidin-5-ylmethyl)imidazo[1,2-a]pyridin-8-amine; 6-O-Phosphoryl Inosine Monophosphate; 6-methoxy-9-methyl[1,3]dioxolo[4,5-h]quinolin-8(9H)-one; 3-CYCLOHEXYL-1-(2-MORPHOLIN-4-YL-2-OXOETHYL)-2-PHENYL-1H-INDOLE-6-CARBOXYLIC ACID; 6-PHENYL[5H]PYRROLO[2,3-B]PYRAZINE; 6-Phenyl-4(R)-(7-Phenyl-Heptanoylamino)-Hexanoic Acid; 7,8,10-TRIMETHYLBENZO[G]PTERIDINE-2,4(3H,10H)-DIONE; 7,8-DICHLORO-1,2,3,4-TETRAHYDROISOQUINOLINE; 7,8-dihydro-6-hydroxymethyl-7-methyl-7-[2-phenylethyl]-pterin; 7,8-Dihydro-7,7-Dimethyl-6-Hydroxypterin; 7-(2,5-dihydropyrrol-1-yl)-6-phenyl-pyrido[6,5-d]pyrimidin-2-amine; 7-(Carboxyamino)-8-Amino-Nonanoic Acid; 6-Oxo-8,9,10,11-Tetrahydro-7h-Cyclohepta[C][1]Benzopyran-3-O-Sulfamate; 7-[(3-CHLOROBENZYL)OXY]-2-OXO-2H-CHROMENE-4-CARBALDEHYDE; 3′-Deoxy 3-Amino Adenosine-5′-Diphosphate; 7-[3-(4-FLUORO-PHENYL)-1-ISOPROPYL-1H-INDOL-2-YL]-3,5-DIHYDROXY-HEPTANOIC ACID; 3-Fluoro-2-(Phosphonooxy)Propanoic Acid; 7-[4-(Dimethylamino)Phenyl]-N-Hydroxy-4,6-Dimethyl-7-Oxo-2,4-Heptadienamide; 7-{2,6-DICHLORO-4-[3-(2-CHLORO-BENZOYL)-UREIDO]-PHENOXY}-HEPTANOIC ACID; 7-{[(CYCLOHEXYLAMINO)CARBONYL]AMINO}HEPTANOIC ACID; 7A-[(4-cyanophenyl)methyl]-6-(3,5-dichlorophenyl)-5-oxo-2,3,5,7A-tetrahydro-1H-pyrrolo[1,2-A]pyrrole-7-carbonitrile; 7-Alpha-D-Ribofuranosyl-2-Aminopurine-5′-Phosphate; 3-fluoro-6-(4-fluorophenyl)-2-hydroxy-6-oxohexa-2,4-dienoic acid; 3-fluoro-N-1H-indol-5-yl-5-morpholin-4-ylbenzamide; 7-amino-2-tert-butyl-4-{[2-(1H-imidazol-4-yl)ethyl]amino}pyrido[2,3-d]pyrimidine-6-carboxamide; 3-Hydroxy-4-(3,4,5-Trihydroxy-Tetrahydro-Pyran-2-Yloxy)-Piperidin-2-One; 7-hydroxy-4-methyl-2H-chromen-2-one; 7-carboxy-5-hydroxy-12,13-dihydro-6H-indolo[2,3-a]pyrrolo[3,4-c]carbazole; 3-FLUORO-4-[2-HYDROXY-2-(5,5,8,8-TETRAMETHYL-5,6,7,8,-TETRAHYDRO-NAPHTALEN-2-YL)-ACETYLAMINO]-BENZOIC ACID; 7-BENZYL-1,3-DIMETHYL-8-PIPERAZIN-1-YL-3,7-DIHYDRO-PURINE-2,6-DIONE; 7-HYDROXY-4-METHYL-3-(2-HYDROXY-ETHYL)COUMARIN; 7-METHOXY-1-METHYL-9H-BETA-CARBOLINE; 7-methoxy-4-[(6-phenyl[1,2,4]triazolo[4,3-b]pyridazin-3-yl)methoxy]quinoline; 7-Methoxy-8-[1-(Methylsulfonyl)-1h-Pyrazol-4-Yl]Naphthalene-2-Carboximidamide; 7-methyl-guanosine-5′-triphosphate-5′-guanosine; 3-FLUORO-5-MORPHOLIN-4-YL-N-[3-(2-PYRIDIN-4-YLETHYL)-1H-INDOL-5-YL]BENZAMIDE; 7n-Methyl-8-Hydroguanosine-5′-Monophosphate; 7n-Methyl-8-Hydroguanosine-5′-Triphosphate; 4-((3r,4s,5r)-4-Amino-3,5-Dihydroxy-Hex-1-Ynyl)-5-Fluoro-3-[1-(3-Methoxy-1h-Pyrrol-2-Yl)-Meth-(Z)-Ylidene]-1,3-Dihydro-Indol-2-One; 8-(2,5-Dimethoxy-Benzyl)-2-Fluoro-9h-Purin-6-Ylamine; 4-(3,12,14-Trihydroxy-10,13-Dimethyl-Hexadecahydro-Cyclopenta[a]Phenanthren-17-Yl)-5h-Furan-2-One; 4-({5[(4-AMINOCYCLOHEXYL)AMINO][1,2,4]TRIAZOLO[1,5-A]PYRIMIDIN-7-YL}AMINO)BENZENESULFONAMIDE; 8-(2-Chloro-3,4,5-Trimethoxy-Benzyl)-9-Pent-4-Ylnyl-9h-Purin-6-Ylamine; 8-(Pyrimidin-2-Ylamino)Naphthalene-2-Carboximidamide; 8,9,10-Trihydroxy-7-Hydroxymethyl-2-Thioxo-6-Oxa-1,3-Diaza-Spiro[4.5]Decan-4-One; 8,9,10-Trihydroxy-7-Hydroxymethyl-3-Methyl-6-Oxa-1,3-Diaza-Spiro[4.5]Decane-2,4-Dione; 8,9-Dichloro-2,3,4,5-Tetrahydro-1h-Benzo[C]Azepine; 4,6-Dideoxy-4-{[4,5,6-Trihydroxy-3-(Hydroxymethyl)Cyclohex-2-En-1-Yl]Amino}-Alpha-D-Lyxo-Hexopyranosyl-(1-&gt;4)-Alpha-D-Threo-Hexopyranosyl-(1-&gt;6)-Alpha-L-Threo-Hexopyranose; 4,6-DIDEOXY-4-{[4-[(4-O-HEXOPYRANOSYLHEXOPYRANOSYL)OXY]-5,6-DIHYDROXY-3-(HYDROXYMETHYL)CYCLOHEX-2-EN-1-YL]AMINO}HEXOPYRANOSYL-(1->4)HEXOPYRANOSYL-(1->4)HEXOPYRANOSE; 8-BENZO[1,3]DIOXOL-,5-YLMETHYL-9-BUTYL-9H-; N-[(CYCLOHEXYLAMINO)CARBONYL]GLYCINE; N-[(Furan-2-Yl)Carbonyl]-(S)-Leucyl-(R)-[1-Amino-2(1h-Indol-3-Yl)Ethyl]-Phosphonic Acid; 4-({[4-(3-METHYLBENZOYL)PYRIDIN-2-YL]AMINO}METHYL)BENZENECARBOXIMIDAMIDE; 4-[(1S,2S,5S)-5-(HYDROXYMETHYL)-8-METHYL-3-OXABICYCLO[3.3.1]NON-7-EN-2-YL]PHENOL; 4-[(2-{4-[(CYCLOPROPYLCARBAMOYL)AMINO]-1H-PYRAZOL-3-YL}-1H-BENZIMIDAZOL-6-YL)METHYL]MORPHOLIN-4-IUM; 8-Oxo-2′-Deoxy-Guanosine-5′-Monophosphate; 4-{[4-{[(1R,2R)-2-(dimethylamino)cyclopentyl]amino}-5-(trifluoromethyl)pyrimidin-2-yl]amino}-N-methylbenzenesulfonamide; 6-chloro-N-pyrimidin-5-yl-3-{[3-(trifluoromethyl)phenyl]amino}-1,2-benzisoxazole-7-carboxamide; 9-(4-hydroxybutyl)-N2-phenylguanine; 9-(3-IODOBENZYLAMINO)-1,2,3,4-TETRAHYDROACRIDINE; 6-(4-Difluoromethoxy-3-Methoxy-Phenyl)-2h-Pyridazin-3-One; 9-(6-Deoxy-Alpha-L-Talofuranosyl)-6-Methylpurine; 6-(5-BROMO-2-HYDROXYPHENYL)-2-OXO-4-PHENYL-1,2-DIHYDROPYRIDINE-3-CARBONITRILE; 9,10-Deepithio-9,10-Didehydroacanthifolicin; 9,9,9-TRI FLUORO-8-OXO-N-PHENYLNONANAMIDE; 9-ACETYL-2,3,4,9-TETRAHYDRO-1H-CARBAZOL-1-ONE; 9-Amino-2-Deoxy-2,3-Dehydro-N-Acetyl-Neuraminic Acid; 6-[(Z)-Amino(Imino)Methyl]-N-[4-(Aminomethyl)Phenyl]-4-(Pyrimidin-2-Ylamino)-2-Naphthamide; 6-FLUORO-2-[2-HYDROXY-3-(2-METHYL-CYCLOHEXYLOXY)-PHENYL]-1H-INDOLE-5-CARBOXAMIDINE; 6-[(Z)-AMINO(IMINO)METHYL]-N-[3-(CYCLOPENTYLOXY)PHENYL]-2-NAPHTHAMIDE; 6-CARBAMIMIDOYL-2-[2-HYDROXY-5-(3-METHOXY-PHENYL)-INDAN-1-YL]-HEXANOIC ACID; 9-Butyl-8-(2,5-Dimethoxy-Benzyl)-2-Fluoro-9h-Purin-6-Ylamine; 6-CARBAMIMIDOYL-4-(3-HYDROXY-2-METHYL-BENZOYLAMINO)-NAPHTHALENE-2-CARBOXYLIC ACID METHYL ESTER; 9-Butyl-8-(2-Chloro-3,4,5-Trimethoxy-Benzyl)-9h-Purin-6-Ylamine; 4-[(7-OXO-7H-THIAZOLO[5,4-E]INDOL-8-YLMETHYL)-AMINO]-N-PYRIDIN-2-YL-BENZENESULFONAMIDE; 6-CHLORO-3-(3-METHYLISOXAZOL-5-YL)-4-PHENYLQUINOLIN-2(1H)-ONE; 4-[(CYCLOPROPYLETHYNYL)OXY]-6-FLUORO-3-ISOPROPYLQUINOLIN-2(1H)-ONE; 4-[(4-METHYL-1-PIPERAZINYL)METHYL]-N-[3-[[4-(3-PYRIDINYL)-2-PYRIMIDINYL]AMINO]PHENYL]-BENZAMIDE; 6-CHLORO-N-{(3S)-1-[(1S)-1-METHYL-2-MORPHOLIN-4-YL-2-OXOETHYL]-2-OXOPYRROLIDIN-3-YL}-1-BENZOTHIOPHENE-2-SULFONAMIDE; 4-{[(CYCLOHEXYLAMINO)CARBONYL]AMINO}BUTANOIC ACID; 4-{[(E)-2-(5-CHLOROTHIEN-2-YL)VINYL]SULFONYL}-1-(1H-PYRROLO[3,2-C]PYRIDIN-2-YLMETHYL)PIPERAZIN-2-ONE; 4-{[(Z)-(5-OXO-2-PHENYL-1,3-OXAZOL-4(5H)-YLIDENE)METHYL]AMINO}BUTANOIC ACID; 4-{[1-METHYL-2,4-DIOXO-6-(3-PHENYLPROP-1-YN-1-YL)-1,4-DIHYDROQUINAZOLIN-3(2H)-YL]METHYL}BENZOIC ACID; 4-{[1-Methyl-5-(2-Methyl-Benzoimidazol-1-Ylmethyl)-1h-Benzoimidazol-2-Ylmethyl]-Amino}-Benzamidine; 4-[(4-O-SULFAMOYLBENZYL)(4-CYANOPHENYL)AMINO]-4H-[1,2,4]-TRIAZOLE; 4-{[4-(4-fluoro-3-methylphenyl)-1,3-thiazol-2-yl]amino}-2-hydroxybenzoic acid; 9-(2-Deoxy-Beta-D-Ribofuranosyl)-6-Methylpurine; 4-{[4-AMINO-6-(CYCLOHEXYLMETHOXY)-5-NITROSOPYRIMIDIN-2-YL]AMINO}BENZAMIDE; 4-{[5-(CYCLOHEXYLAMINO)[1,2,4]TRIAZOLO[1,5-A]PYRIMIDIN-7-YL]AMINO}BENZENESULFONAMIDE; 4-{[5-(CYCLOHEXYLOXY)[1,2,4]TRIAZOLO[1,5-A]PYRIMIDIN-7-YL]AMINO}BENZENESULFONAMIDE; and 4-{[5-(CYCLOHEXYLMETHOXY)[1,2,4]TRIAZOLO[1,5-A]PYRIMIDIN-7-YL]AMINO}BENZENESULFONAMIDE

Additional perturbations/drugs which may be utilized include, but are not limited to, abacavir; abarelix; abatacept; abatacept; abciximab; abequose; abiraterone; acadesine; acamprosate; acarbose; acarbose derived hexasaccharide; acebutolol; acediasulfone; acenocoumarol; acepromazine; aceprometazine; acetamide; acetaminophen; acetazolamide; acetic acid; acetic acid salicyloyl-amino-ester; acetoacetic acid; acetoacetyl-coenzyme a; acetohexamide; acetohydroxamic acid; acetone cyanohydrin; acetophenazine; acetophenone; acetorphine; acetrizoic acid; acetyl dithranol; acetylamino-acetic acid; acetylcarnitine; acetylcholine; acetylcysteine; acetyldigitoxin; acetyldihydrocodeine; acetylgalactosamine-4-sulfate; acetylphosphate; acetylsalicylic acid; aciclovir; acitretin; aclarubicin; aclidinium; aconitate ion; acrivastine; acrylic acid; activated charcoal; actyve hormone therapy; acylated ceftazidime; adalimumab; adamantane; adamantanone; adapalene; adefovir dipivoxil; adenine; adenosine; adenosine monophosphate; adenosine monotungstate; adenosine phosphonoacetic acid; adenosine triphosphate; adenosine-2′-5′-diphosphate; adenosine-3-5′-diphosphate; adenosine-5′-(dithio)phosphate; adenosine-5-diphosphoribose; adenosine-5′-ditungstate; adenosine-5′-pentaphosphate; adenosine-5′-phosphosulfate; adenosine-5′-propylphosphate; adenylosuccinic acid; adinazolam; ado-p-ch2-p-ps-ado; adrafinil; aerolef; aeruginosin 98-b; aetiocholanolone; afamelanotide; afatinib; afelimomab; afimoxifene; aflibercept; agalsidase beta; agmatine; agomelatine; aicar; ajmaline; alatrofloxacin; albendazole; albiglutide; albumin-interferon alpha; alcaftadine; alclometasone; aldesleukin; aldosterone; alectinib; alefacept; aleglitazar; alemtuzumab; alendronic acid; alfacalcidol; alfentanil; alferminogene tadenovec; alfimeprase; alfuzosin; alglucerase; alglucosidase alfa; alimemazine; alirocumab; aliskiren; alitretinoin; alizapride; alkaline phosphatase; alto-isoleucine; allolactose; allopurinol; allosamidin; allosamizoline; allylestrenol; allylprodine; almitrine; almotriptan; alogliptin; alosetron; alpha chlorophyll a; alphacetylmethadol; alpha-d-fucose; alpha-d-xylopyranose; alpha-ethyltryptamine; alpha-ketoisovalerate; alpha-ketomalonic acid; alpha-1-arabinose; alpha-1-fucose; alpha-1-iduronic acid; alpha-linolenic acid; alpha-1-methyl-fucose; alpha-maltotriose; alphameprodine; alphamethadol; alprazolam; alprazolam lingual spray; alprenolol; alprostadil; alrestatin; alseroxylon; alsterpaullone; alteplase; altretamine; altropane; aluminium; aluminium monostearate; aluminum hydroxide; alverine; alvimopan; amantadine; ambenonium; ambrisentan; ambroxol; amcinonide; amdinocillin; amedalin; amfecloral; amido phenyl pyruvic acid; amifostine; amikacin; amiloride; aminacrine; amineptine; aminoanthracene; aminocandin; aminocaproic acid; aminoglutethimide; aminoguanidine; aminohippuric acid; aminolevulinic acid; aminomethylcyclohexane; aminooxyacetic acid; aminophenazone; aminophosphonic acid-guanylate ester; aminophylline; aminopterin; aminorex; aminosalicylic acid; amiodarone; amisulpride; amitriptyline; amlexanox; amlodipine; ammonia n-13; ammonium chloride; ammonium lactate; amobarbital; amodiaquine; amolimogene; amonafide; amoxapine; amoxicillin; amperozide; amphetamine; amphotericin b; ampicillin; amprenavir; amrinone; amrubicin; amsacrine; amyl nitrite; amylamine; amylocaine; anagrelide; anakinra; analogue of indinavir drug; anastrozole; anatibant; ancestim; ancrod; androstanedione; androstenediol; anecortave acetate; anhydrous dextrose; anidulafungin; anileridine; aniline; aniracetam; anisindione; anisomycin; anisotropine methylbromide; anistreplase; antazoline; antiproliferative agent a771726; antipyrine; antithrombin alfa; antithrombin iii human; anti-thymocyte globulin (equine); anti-thymocyte globulin (rabbit); antrafenine; apadenoson; apixaban; aplyronine a; apomorphine; apraclonidine; apramycin; apremilast; aprepitant; aprindine; aprobarbital; aprotinin; apstatin; aptazapine; ara-alpha(1,3)-xyl; arabinose-5-phosphate; arachidonic acid; aranidipine; arbaclofen; arbaclofen placarbil; arbekacin; arbutamine; arcitumomab; ardeparin; arecoline; arformoterol; argadin; argatroban; argifin; arginineamide; argininosuccinate; arimoclomol; aripiprazole; armodafinil; arotinolol; arsanilic acid; arsenic trioxide; arsthinol; artemether; artesunate; articaine; asenapine; asfotase alfa; asimadoline; asparaginase; asparaginase Erwinia chrysanthemi; aspartame; aspartate beryllium trifluoride; aspartate semialdehyde; aspartic acid-4-carboxymethyl ester; aspartyl-adenosine-5′-monophosphate; astemizole; ataluren; atamestane-plus-toremifene; atazanavir; atenolol; atezolizumab; atg-fresenius s; atomoxetine; atorvastatin; atovaquone; atpenin a5; atracurium besylate; atrazine glutathione conjugate; atropine; attapulgite; auranofin; aurodox; aurothioglucose; aurovertin b; avanafil; avibactam; avobenzone; axitinib; azacitidine; azapropazone; azatadine; azathioprine; azelaic acid; azelastine; azelnidipine; azidocillin; azilsartan medoxomil; azimilide; azithromycin; azlocillin; azo-dye hapten; azosemide; aztreonam; b-2-octylglucoside; bacampicillin; bacitracin; baclofen; bacteriochlorophyll a; bafilomycin al; bafilomycin b1; balanol; balanol analog 1; balanol analog 2; balanol analog 8; balhimycin; balsalazide; bambuterol; banoxantrone; barbexaclone; barbital; barbituric acid derivative; barnidipine; basiliximab; batimastat; bavituximab; bazedoxifene; becaplermin; beclamide; beclomethasone dipropionate; becocalcidiol; bectumomab; bedaquiline; beeswax; befunolol; belatacept; belimumab; belinostat; bemiparin; benactyzine; benazepril; bendamustine; bendroflumethiazide; benfluorex; benidipine; benmoxin; benoxaprofen; bentiromide; bentoquatam; benzamidine; benzathine benzylpenicillin; benzatropine; benzene hexacarboxylic acid; benzenesulfonyl; benzethidine; benzhydroxamic acid; benzimate; benzimidazole; benzo[b]thiophene-2-boronic acid; benzo[b]thiophene-2-carboxamidine; benzocaine; benzoctamine; benzofuran; benzoic acid; benzonatate; benzophenone; benzothiazole; benzoyl peroxide; benzoyl-arginine-alanine-methyl ketone; benzoylecgonine; benzoylformic acid; benzphetamine; benzquinamide; benzthiazide; benzydamine; benzyl (2-oxopropyl)carbamate; benzyl alcohol; benzyl benzoate; benzylamine; benzylcysteine; benzylfentanyl; benzylmorphine; benzylpenicillin; benzylpenicilloyl polylysine; benzylsulfinic acid; bepotastine; bepridil; beractant; beraprost; berberine; besifloxacin; beta alethine; beta-(1-&gt;4)-galactotriose; beta-(1->4)-galactotriose; beta-(2-naphthyl)-alanine; beta(2-thienyl)alanine; beta-3-cysteine; beta-3-serine; beta-alanine; beta-amino isobutyrate; beta-cellotriose; betacetylmethadol; beta-cyclohexyl-alanine; beta-d-fructopyranose; beta-d-fucose; beta-d-galactose 6-phosphate; beta-d-glucose; beta-d-glucose 6-phosphate; beta-d-ribopyranose; betahistine; beta-hydroxyasparagine; beta-hydroxyaspartic acid; beta-hydroxyfentanyl; beta-hydroxyleucine; beta-hydroxytryptophane; beta-1-arabinose; beta-1-fucose; beta-1-methyl-fucose; beta-mannobiose; betameprodine; beta-mercaptoethanol; betamethasone; beta-methyllactoside; beta-naphthoflavone; betaprodine; beta-propiolactone; betaxolol; betazole; bethanechol; bethanidine; bevacizumab; bevantolol; bevirimat; bexarotene; bezafibrate; bezitramide; bicalutamide; bicifadine; bicine; bicuculline; bifeprunox; bifonazole; bilastine; biliverdine ix alpha; bimatoprost; binodenoson; biopterin; biotin; biotino1-5-amp; biotinyl p-nitroaniline; biperiden; biphenyl-2,3-diol; biphenylalanine; biricodar dicitrate; bisacodyl; bis-benzamidine; bishydroxy[2h-1-benzopyran-2-one,1,2-benzopyrone]; bismuth subcitrate; bismuth subsalicylate; bis-napthyl beta-ketophosphonic acid; bisoprolol; bisoxatin; bithionol; bitolterol; bivalirudin; bleomycin; blinatumomab; blonanserin; boceprevir; bolasterone; boldenone; bombykol; bopindolol; bortezomib; bosentan; bosutinib; botulinum toxin type a; botulinum toxin type b; bradanicline; brasofensine; br-coeleneterazine; brecanavir; brentuximab vedotin; brequinar analog; bretylium; brexpiprazole; briakinumab; brifentanil; brimonidine; brinzolamide; brodimoprim-4,6-dicarboxylate; bromamphenicol; bromazepam; bromfenac; bromhexine; bromocriptine; bromodiphenhydramine; bromo-dodecanol; bromopride; bromopurine; bromo-willardiine; brompheniramine; brotizolam; buclizine; budesonide; buformin; bufotenine; bufuralol; bulgecin a; bumetanide; bunamiodyl; bupivacaine; bupranolol; buprenorphine; bupropion; buserelin; buspirone; busulfan; butabarbital; butalbital; butan-1-ol; butanoic acid; butenafine; butenoic acid; butethal; butoconazole; butorphanol; butriptyline; butylamine; butylphosphonate; butyramide; butyrfentanyl; butyrylthiocholine; cabazitaxel; cabergoline; cabozantinib; caffeine; calanolide a; calcidiol; calcipotriol; calcitriol; calcium; calcium acetate; calcium carbimide; calcium carbonate; calcium chloride; calcium gluceptate; calfactant; calusterone; calyculin a; camazepam; camphane; camphor; camptothecin; canagliflozin; canakinumab; canaline; candesartan; candicidin; candoxatril; candoxatrilat; canfosfamide; cangrelor; cannabidiol; cannabinor; canrenoic acid; capecitabine; capreomycin; capromab; caprospinol; caprylic acid; capsaicin; captodiame; captopril; carbachol; carbamazepine; carbamic acid; carbaphosphonate; carbazochrome; carbazole butanoic acid; carbenicillin; carbenoxolone; carbetocin; carbidopa; carbimazole; carbinoxamine; carbocisteine; carbon dioxide; carbon monoxide; carboplatin; carboprost tromethamine; carboxin; carboxyatractyloside; carboxyethyllumazine; carboxylic prpp; carboxymycobactin s; carboxymycobactin t; cardiolipin; carfentanil; carfilzomib; carglumic acid; cariprazine; carisoprodol; carmofur; carmustine; caroxazone; carphenazine; carprofen; carpropamide; carteolol; carvedilol; caspofungin; castanospermine; cat hair allergenic extracts; cathine; cathinone; cefacetrile; cefaclor; cefadroxil; cefalotin; cefamandole; cefapirin; cefazolin; cefdinir; cefditoren; cefepime; cefixime; cefmenoxime; cefmetazole; cefonicid; cefoperazone; ceforanide; cefotaxime; cefotaxime group; cefotetan; cefotiam; cefoxitin; cefpiramide; cefpodoxime; cefprozil; cefradine; cefroxadine; ceftaroline fosamil; ceftazidime; ceftibuten; ceftizoxime; ceftobiprole; ceftolozane; ceftriaxone; cefuroxime; celecoxib; celiprolol; cellobiose; cellotetraose; cephalexin; cephaloglycin; cephaloridine; cephalosporin analog; cephalosporin c; cephalothin group; ceritinib; cerivastatin; certolizumab pegol; certoparin; cerulenin; ceruletide; cethromycin; cetirizine; cetrimonium; cetrorelix; cetuximab; cevimeline; chenodeoxycholic acid; chitotriose; chloral betaine; chloral hydrate; chlorambucil; chloramphenicol; chlorcyclizine; chlordiazepoxide; chlorhexadol; chlorhexidine; chlormerodrin; chlormezanone; chloro diiron-oxo moiety; chlorophyll a; chlorophyll b; chloroprocaine; chloropyramine; chloroquine; chlorothiazide; chlorotrianisene; chloroxine; chlorphenamine; chlorphenesin; chlorphenoxamine; chlorphentermine; chlorpromazine; chlorpropamide; chlorprothixene; chlortetracycline; chlorthalidone; chlorzoxazone; cholecalciferol; cholecystokinin; cholesterol; cholesterol-sulfate; cholesteryl linoleate; cholestyramine; cholic acid; choline; choline alfoscerate; choline c 11; choline c-11; chondroitin sulfate; chromic chloride; chromophore (asp-tyr-gly); chromophore (glu-tyr-gly); chromophore (gly-tyr-gly); chromophore (his-tyr-gly); chromophore (lys-tyr-gly); chromophore (met-tyr-gly); chymostatin; cibacron blue; ciclesonide; ciclopirox; cidofovir; ciglitazone; cilansetron; cilastatin; cilazapril; cilnidipine; cilomilast; cilostazol; cimetidine; cimetropium; cimicoxib; cinacalcet; cinalukast; cinchocaine; cinitapride; cinnarizine; cinolazepam; cinoxacin; cintredekin besudotox; ciprofloxacin; cirazoline; cisapride; cisatracurium besylate; cisplatin; cis-tetracosenoyl sulfatide; citalopram; citraconic acid; citric acid; cladribine; clarithromycin; clavulanate; clemastine; clenbuterol; clevidipine; clidinium; clindamycin; clinofibrate; clioquinol; clobazam; clobetasol propionate; clobutinol; clocapramine; clocortolone; clodronate; clofarabine; clofazimine; clofedanol; clofibrate; clomifene; clomipramine; clomocycline; clonazepam; clonidine; clonitazene; clonixin; cloperastine; clopidogrel; clorazepate; clorobiocin; clorocruoro hem; clortermine; clostebol; clotiazepam; clotrimazole; cloxacillin; cloxazolam; clozapine; coa-s-acetyl tryptamine; coa-s-trimethylene-acetyl-tryptamine; cobalt hexammine ion; cobicistat; cobimetinib; cocaine; Coccidioides immitis spherule; codeine; codeine methylbromide; codeine-n-oxide; coelenteramide; coenzyme a; coenzyme a persulfide; coenzyme f420; coenzyme q10; colchicine; colesevelam; colestipol; colforsin; colfosceril palmitate; colistimethate; colistin; conivaptan; conjugated equine estrogens; copper; coprogen; coproporphyrin coproporphyrin containing co(iii); coproporphyrin iii; cordycepin triphosphate; corticorelin ovine triflutate; corticotropin; corticotropin-releasing factor; cortisone acetate; cosyntropin; cp-coeleneterazine; crc200 (chiron-behring); creatine; crizotinib; crofelemer; cromoglicic acid; cronidipine; crotamiton; crotonaldehyde; cryopreserved human liver cells; cryptenamine; cu-bicyclam; cu-cyclam; cupric chloride; cyamemazine; cyanamide; cyanocinnoline; cyanocobalamin; cyclacillin; cyclandelate; cyclizine; cyclobenzaprine; cyclo-hepta-amylose; cyclohexane propionic acid; cyclohexanol; cyclohexanone; cyclohexylammonium ion; cyclohexylformamide; cyclohexyl-norstatine; cyclohexyl-pentyl-maltoside; cycloleucine; cyclopamine; cyclopentamine; cyclopentolate; cyclophosphamide; cycloserine; cyclosporine; cyclo-tetrametavanadate; cyclotheonamide a; cyclothiazide; cyclouridine; cycrimine; cyprenorphine; cyproheptadine; cyproterone acetate; cysteamine; cysteine hydrochloride; cysteine-s-acetamide; cysteinesulfonic acid; cystein-s-yl cacodylate; cyt006-angqb; cytarabine; cytidine; cytidine 5′-diphosphoglycerol; cytidine-3-monophosphate; cytidine-5′-diphosphate; cytidine-5′-diphospho-beta-d-xylose; cytidine-5′-monophosphate; cytidine-5′-monophosphate-5-n-acetylneuraminic acid; cytidine-5′-triphosphate; cytidyl-2′-5′-phospho-guanosine; cytisine; cytosine arabinose-5′-phosphate; d-1,4-dithiothreitol; d-2-keto-3-deoxygalactonate; d-2-keto-3-deoxygluconate; d-4-phosphoerythronic acid; dabigatran etexilate; dabrafenib; dacarbazine; daclatasvir; daclizumab; dactinomycin; daidzin; d-alanine; dalbavancin; daledalin; dalfampridine; dalfopristin; d-allopyranose; dalteparin; danazol; dansylamide; dansyllysine; dantrolene; dantron; dapagliflozin; dapiprazole; dapoxetine; dapsone; daptomycin; daratumumab; darbepoetin alfa; d-arginine; darifenacin; darodipine; darunavir; darusentan; dasabuvir; dasatinib; d-asparagine; d-aspartic acid; daunorubicin; dazoxiben; dcka, 5,7-dichlorokynurenic acid; d-cysteine; d-dethiobiotin; deacetoxycephalosporin-c; deamido-nad+; deamino-methyl-phenylalanine; debrisoquin; debromohymenialdisine; decamethonium; decane-1-thiol; decanoic acid; decitabine; decyl formate; decyloxy-methanol; deferasirox; deferiprone; deferoxamine; defibrotide; degarelix; deglucobalhimycin; degraded cephaloridine; dehydroascorbic acid; dehydrochloromethyltestosterone; dehydrocholic acid; dehydroepiandrosterone; dehydroepiandrosterone sulfate; delavirdine; delorazepam; delta1-dihydrotestosterone; demecarium; demeclocycline; demexiptiline; dendritic cell therapy; denileukin diftitox; denosumab; denufosol; deoxy-2-fluoro-b-d-cellotrioside; deoxycholic acid; deoxycytidylyl-3′,5′-guanosine; deoxyguanidinoproclavaminic acid; deoxythymidine; deoxyuridine-5′-diphosphate; deoxyuridine-5-triphosphate; dephospho coenzyme a; dequadin; dequalinium; d-eritadenine; descarboxy-nor-n(omega)-hydroxy-1-arginine; deserpidine; desflurane; desipramine; desirudin; deslanoside; desloratadine; desmethylprodine; desmopressin; desmoteplase; desogestrel; desomorphine; desonide; desoximetasone; desoxycorticosterone pivalate; desulfo-coenzyme a; desvancosaminyl vancomycin; desvenlafaxine; dexamethasone; dexbrompheniramine; dexchlorpheniramine maleate; dexetimide; dexfenfluramine; dexibuprofen; dexketoprofen; dexloxiglumide; dexmedetomidine; dexmethylphenidate; dexpanthenol; dexrazoxane; dextran; dextran 40; dextroamphetamine; dextrofloxacine; dextromethorphan; dextromoramide; dextropropoxyphene; dextrothyroxine; dezocine; d-galactohydroximo-1,5-lactam; d-galctopyranosyl-1-on; d-glucitol 6-phosphate; d-gluconhydroximo-1,5-lactam; d-glucose in linear form; d-glucuronic acid; d-glutamic acid; d-glutamine; dha-paclitaxel; di(n-acetyl-d-glucosamine); diacetyldeuteroheme; diampromide; diatrizoate; diazepam; diazoxide; dibenzofuran-4,6-dicarboxylic acid; dibromothymoquinone; dichloralphenazone; dichloroacetic acid; diclofenac; (diaminomethyl-methyl-amino)-acetic acid; (diphosphono)aminophosphonic acid; diclofenamide; diclosan; dicloxacillin; dicoumarol; dicyclomine; didanosine; didecyl-dimethyl-ammonium; dienestrol; dienogest; diethylcarbamazine; diethylcarbamodithioic acid; diethylphosphono group; diethylpropion; diethylstilbestrol; diethylthiambutene; diethyltryptamine; difenoxin; diflorasone; diflunisal; difluocortolone; difluoromethionine; difluprednate; digalactosyl diacyl glycerol (dgdg); digitoxin; digoxin; digoxin immune fab (ovine); diguanosine-5′-triphosphate; dihomo-gamma-linolenic acid; dihydro-acarbose; dihydrocodeine; dihydroergotamine; dihydroetorphine; dihydrofolic acid; dihydrogenphosphate ion; dihydrolipoic acid; dihydromorphine; dihydroorotic acid; dihydroquinidine barbiturate; dihydrotachysterol; dihydrotestosterone; dihydroxyacetone; diiodohydroxyquinoline; diisopropylphosphono group; di-linoleoyl-3-sn-phosphatidylcholine; diloxanide; diltiazem; dimenhydrinate; dimenoxadol; dimercaprol; dimetacrine; dimethyl fumarate; dimethyl propionate ester heme; dimethyl sulfoxide; dimethyl thiophosphate; dimethylallyl diphosphate; dimethylallyl s-thiolodiphosphate; dimethylformamide; dimethylglycine; dimethylthiambutene; dimethyltryptamine; dimetindene; dimetotiazine; diminazene; dinitrophenylene; dinoprost tromethamine; dinoprostone; dinor-n(omega)-hydroxy-l-arginine; dinutuximab; diosmin; dioxaphetyl butyrate; dioxothiomolybdenum(vi) ion; dioxyselenocysteine; diphemanil methylsulfate; diphenhydramine; diphenidol; diphenoxylate; diphenylacetic acid; diphenylpyraline; diphosphate; diphthamide; dipicolinic acid; dipipanone; dipivefrin; dipotassium phosphate; diprenorphine; dipyridamole; dipyrromethane cofactor; dirithromycin; disodium phosphate; disopyramide; d-isovaline; disulfiram; ditazole; dithiane diol; dithioerythritol; diundecyl phosphatidyl choline; diureido-acetate; d-lactic acid; d-leucine; d-limonene 1,2-epoxide; d-lysine; d-mannose 1-phosphate; d-mannuronic acid; d-methionine; dobutamine; docetaxel; docosa-4,7,10,13,16,19-hexaenoic acid; docosanol; dodecane-trimethylamine; dodecyl sulfate; dodecyl-alpha-d-maltoside; dodecyl-coa; dofetilide; dolasetron; dolutegravir; domiphen; domoic acid; domperidone; donepezil; dopamine; doripenem; dornase alfa; dorzolamide; dosulepin; double oxidized cysteine; doxacurium chloride; doxapram; doxazosin; doxepin; doxercalciferol; doxofylline; doxorubicin; doxorubicin transdrug; doxycycline; doxylamine; d-phenylalanine; d-proline; dronabinol; dronedarone; droperidol; drospirenone; drostanolone; drotaverine; drotebanol; drotrecogin alfa; droxicam; droxidopa; d-serine; d-tartaric acid; d-threo-neopterin; d-threonine; d-treitol; d-tryptophan; d-tyrosine; dulaglutide; duloxetine; duramycin; duroquinone; dust mite allergen extracts; dutasteride; duvoglustat; d-xylitol; d-xylulose; dyclonine; dydrogesterone; dyphylline; ecabet; ecallantide; ecamsule; ecgonine; ecgonine methyl ester; echothiophate; econazole; eculizumab; edetic acid; edivoxetine; edotecarin; edoxaban; edrophonium; efalizumab; efavirenz; efinaconazole; eflornithine; efmoroctocog alfa; efonidipine; eftrenonacog alfa; eirna (expressed interfering ma); elacridar; elafin; elaidoylamide; elbasvir; eldecalcitol; elesclomol; eletriptan; eliglustat; ellagic acid; elosulfase alfa; elotuzumab; elsamitrucin; eltrombopag; eluxadoline; elvitegravir; embutramide; emedastine; empagliflozin; emtricitabine; enalapril; enalaprilat; enalkiren; encainide; enclomiphene; enflurane; enfuvirtide; eniluracil; enoxacin; enoxaparin; enoximone; enprofylline; entacapone; entecavir; enviomycin; enzalutamide; eperisone; ephedra; ephedrine; epibatidine; epicept np-1; epigallocatechin; epinastine; epinephrine; epirizole; epirubicin; eplerenone; epoetin zeta; epoprostenol; epothilone b; epothilone d; epratuzumab; eprazinone; eprosartan; eptifibatide; equilenin; equilin; erdosteine; ergocalciferol; ergoloid mesylate; ergonovine; ergosterol; ergotamine; eribulin; eritoran; erlotinib; ertapenem; erythose-4-phosphate; erythrityl tetranitrate; erythromycin; erythropoietin; escitalopram; eslicarbazepine acetate; esmirtazapine; esmolol; esomeprazole; estazolam; estradiol; estramustine; estriol; estrogens, esterified; estrone; estrone sulfate; eszopiclone; etacrynic acid; etamivan; etanercept; eteplirsen; ethambutol; ethanesulfonic acid; ethanol; ethanolamine; ethanolamine oleate; ethchlorvynol; etheno-nad; etheno-nadp; ethinamate; ethinyl estradiol; ethiodized ethionamide; ethoheptazine; ethopropazine; ethosuximide; ethotoin; ethoxzolamide; ethyl biscoumacetate; ethyl carbamate; ethyl dihydrogen diphosphate; ethyl dihydrogen phosphate; ethyl dimethyl ammonio propane sulfonate; ethyl isocyanide; ethyl loflazepate; ethyl oxo(piperidin-1-yl)acetate; ethylaminobenzylmethylcarbonyl group; ethyl-carbamic acid methyl ester; ethylene dichloride; ethylestrenol; ethylisothiourea; ethylmercurithiosalicylic acid; ethylmethylthiambutene; ethylmorphine; ethyl-trimethyl-silane; ethynodiol diacetate; etidocaine; etidronic acid; etifoxine; etilefrine; etiprednol dicloacetate; etizolam; etodolac; etofenamate; etofibrate; etomidate; etonitazene; etonogestrel; etoperidone; etoposide; etoricoxib; etorphine; etoxeridine; etozoline; etravirine; etretinate; eucalyptol; eugenol; everolimus; evolocumab; exemestane; exenatide; ezetimibe; ezogabine; factor iiim; factor ix complex (human); famciclovir; famotidine; famoxadone; farletuzumab; farnesol; farnesyl diphosphate; farnesyl thiopyrophosphate; faropenem medoxomil; febuxostat; felbamate; felodipine; felypressin; fe-mesopone; fenbufen; fencamfamine; fendiline; fenethylline; fenfluramine; fenofibrate; fenoldopam; fenoprofen; fenoterol; fenproporex; fenretinide; fenspiride; fentanyl; fentonium; ferric carboxymaltose; ferric citrate; ferric pyrophosphate; ferricrocin-iron; feruloyl coenzyme a; fesoterodine; fexaramine; fexofenadine; fibrinogen concentrate (human); fibrinolysin; fidarestat; fidaxomicin; filaminast; filgrastim; filgrastim-sndz; fimasartan; finafloxacin; finasteride; fingolimod; firocoxib; flavopiridol; flavoxate; flecainide; fleroxacin; flibanserin; floctafenine; f-loop of vitamin b12; florantyrone; florbetaben (18f); florbetapir (18f); floxuridine; flubendazole; fluclorolone acetonide; flucloxacillin; fluconazole; flucytosine; fludarabine; fludeoxyglucose (18f); fludeoxyglucose f-18; fludiazepam; fludrocortisone; flufenamic acid; flumazenil; flumequine; flumethasone; flunarizine; flunisolide; flunitrazepam; fluocinolone acetonide; fluocinonide; fluocortolone; fluorescein; fluoresceinylthioureido; fluorescin; fluorometholone; fluoro-phosphite ion; fluorotryptophane; fluorouracil; fluoro-willardiine; fluoxetine; fluoxymesterone; flupentixol; fluphenazine; flupirtine; fluprednidene; flurandrenolide; flurazepam; flurbiprofen; flurbiprofen methyl ester; flurothyl; fluspirilene; flutamide; flutemetamol (18f); fluticasone furoate; fluticasone propionate; fluvastatin; fluvoxamine; folic acid; follitropin alpha; follitropin beta; fomepizole; fominoben; fondaparinux sodium; fontolizumab; forasartan; formaldehyde; formebolone; formestane; formic acid; formic acid benzyl ester; formoterol; formycin; formycin b; formycin-5′-monophosphate; fosamprenavir; fosaprepitant; foscarnet; fosfomycin; fosinopril; fosmidomycin; fosphenytoin; fospropofol; fotemustine; framycetin; frovatriptan; fructose; fructose-6-phosphate; fructose-6-phosphate; fucitol; fulvestrant; fumagillin; fumarate; furazabol; furazolidone; furethidine; furo[2,3d]pyrimidine antifolate; furosemide; furoyl-leucine; fursultiamine; fusafungine; fusicoccin; fusidic acid; gabaculine; gabapentin; gabapentin enacarbil; gaboxadol; gadobenic acid; gadobutrol; gadodiamide; gadofosveset trisodium; gadopentetate dimeglumine; gadoteric acid; gadoteridol; gadoversetamide; gadoxetic acid; galactose grease; galactose-uridine-5′-diphosphate; galacturonic acid; galantamine; galiximab; gallamine triethiodide; gallichrome; gallium citrate ga-67; gallium maltolate; gallium nitrate; galsulfase; gamma hydroxybutyric acid; gamma(amino)-butyric acid; gamma-arsono-beta, gamma-methyleneadenosine-5′-diphosphate; gamma-butyrolactone; gamma-carboxy-glutamic acid; gamma-glutamyl[s-(2-iodobenzyl)cysteinyl]glycine; gamma-glutamylcysteine; gamma-phenyl-butyric acid; ganaxolone; ganciclovir; ganirelix; gantacurium chloride; gastric intrinsic factor; gatifloxacin; gavestinel; gefitinib; geldanamycin; gemcabene; gemcitabine; gemeprost; gemfibrozil; gemifloxacin; gemtuzumab ozogamicin; geneticin; genistein; gentamicin; gentamicin c1a; gentian violet; genz-112638; geranyl diphosphate; geranylgeranyl diphosphate; gestodene; gestrinone; ghavamiol; gibberellin a3; gibberellin a4; gimeracil; Ginkgo biloba; ginkgolide-a; ginkgolide-b; ginkgolide-c; ginkgolide-j; ginkgolide-m; ginseng; ginsenoside c; ginsenoside rb1; ginsenoside rg1; glatiramer acetate; glibornuride; gliclazide; glimepiride; glipizide; gliquidone; glisoxepide; glucagon recombinant; glucarate; glucarpidase; gluconic acid; gluconolactone; gluco-phenylimidazole; glucosamine; glucosamine 1-phosphate; glucosamine 4-phosphate; glucosamine 6-phosphate; glucosaminyl-(alpha-6)-d-myo-inositol; glucose; glucose-6-phosphate; glutamine hydroxamate; glutamine t-butyl ester; glutamyl group; glutaric acid; glutathione; glutathione s-(2,4 dinitrobenzene); glutathione sulfinate; glutathione sulfonic acid; glutathionylspermidine; glutathionylspermidine disulfide; glutethimide; glyburide; glyceraldehyde-3-phosphate; glycerin; glycerol; glycerol phenylbutyrate; glycerol-2-phosphate; glycinamid; glycinamide ribonucleotide; glycine; glycine betaine; glycochenodeoxycholic acid; glycodiazine; glycoluril; glycopyrronium; glyoxalate, glyoxylate; glyphosate; gnrh pharmaccine; golimumab; gonadorelin; goserelin; gramicidin d; granisetron; grass pollen extract; grazoprevir; grepafloxacin; griseofulvin; guaifenesin; guanabenz; guanadrel; guanethidine; guanfacine; guanidine; guanidine-3-propanol; guanidinoethylmercaptosuccinic acid; guanine; guanosine; guanosine-2′,3-cyclophosphorothioate; guanosine-2-monophosphate; guanosine-3-monophosphate; guanosine-3-monophosphate-5′-diphosphate; guanosine-5,3-tetraphosphate; guanosine-5′-diphosphate; guanosine-5′-diphosphate-rhamnose; guanosine-5′-monophosphate; guanosine-5′-triphosphate; guvacine; h type trisaccharide; h type ii trisaccharide; hadacidin; halazepam; halcinonide; halofantrine; halofuginone; haloperidol; haloprogin; halothane; haloxazolam; heat-activated liposome technology; helium; heme c; heme d; hemi-babim; hemin; heparin; heparin disaccharide iii-s; heparin disaccharide i-s; hepatitis b immune globulin; heptabarbital; heptamolybdate; heptanamide; heptane-1,2,3-triol; heptanoic acid; heptanyl-p-phenol; heptulose-2-phosphate; heptyl 1-thiohexopyranoside; heptyl-beta-d-glucopyranoside; heroin; hesperetin; hesperidin; hetacillin; hexachlorophene; hexadecanal; hexadecyl octanoate; hexafluoroacetone hydrate; hexafluronium; hexamethonium; hexamethylenetetramine; hexaminolevulinate; hexane-1,6-diol; hexanoyl-coenzyme a; hexatantalum dodecabromide; hexestrol; hexetidine; hexobarbital; hexocyclium; hexoprenaline; hexylcaine; hg9a-9, nonanoyl-n-hydroxyethylglucamide; histamine; histamine dihydrochloride; histamine phosphate; histidinol; histidyl-adenosine monophosphate; histrelin; homatropine methylbromide; homophenylalaninylmethane; homosalate; homoserine lactone; hspe7; hsv-2 theracine; human calcitonin; human Clostridium tetani toxoid immune globulin; human rabies virus immune globulin; human rho(d) immune globulin; human serum albumin; human varicella-zoster immune globulin; humanized smart anti-il-12 antibody; humax-egfr; huperzine a; huperzine b; huperzine-a; hyaluronic acid; hyaluronidase; hyaluronidase (human recombinant); hybrid between b and c type hemes (protoporphyrin ixcontaining fe); hydantocidin-5′-phosphate; hydracarbazine; hydralazine; hydrochlorothiazide; hydrocodone; hydrocortamate; hydrocortisone; hydroflumethiazide; hydrogenobyrinic acid; hydrolyzed cephalothin; hydromorphinol; hydromorphone; hydroquinone; hydroxocobalamin; hydroxyacetic acid; hydroxyaminovaline; hydroxyamphetamine hydrobromide; hydroxychloroquine; hydroxydimethylarsine oxide; hydroxydione; hydroxyethyl cellulose; hydroxyethyl starch; hydroxyethylcysteine; hydroxyfasudil; hydroxyphenyl propionic acid; hydroxyprogesterone caproate; hydroxyproline; hydroxypropyl cellulose; hydroxystilbamidine isethionate; hydroxyurea; hydroxyzine; hymenialdisine; hyoscyamine; hyperforin; hypophosphite; hypoxanthine; i±-methylacetylfentanyl; i±-methylfentanyl; i±-methylthiofentanyl; i²-hydroxythiofentanyl; i²-methylfentanyl; ibandronate; ibritumomab tiuxetan; ibrutinib; ibudilast; ibuprofen; ibuproxam; Ibutilide; icatibant; icodextrin; i-coeleneterazine; icosapent; icosapent ethyl; idarubicin; idarucizumab; idebenone; idelalisib; idoxuridine; idursulfase; ifenprodil; Ifosfamide; iloperidone; iloprost; imatinib; imazaquin; imexon; imidafenacin; imidazole-derived cellobiose; imidazopyridazin 1; imiglucerase; imino-tryptophan; imipenem; imipramine; imiquimod; immucillin-g; immune globulin human; imolamine; implitapide; incadronic acid; indacaterol; indalpine; indane-5-sulfonamide; indapamide; indecainide; indenolol; indinavir; indirubin-3-monoxime; indirubin-5-sulphonate; indium (111in) imciromab; indium in-111 chloride; indium in-111 oxyquinoline; indium in-111 pentetate disodium; indocyanine green; indole; indole naphthyridinone; indole-3-glycerol phosphate; indole-3-propanol phosphate; indolylpropionic acid; indomethacin; indoprofen; indoramin; inecalcitol; infliximab; ingenol mebutate; inhibitor bea322; inhibitor bea369; inhibitor bea388; inhibitor bea403; inhibitor bea409; inhibitor bea425; inhibitor bea428; inhibitor idd 384; inhibitor msa367; inhibitor of p38 kinase; inhibitor q8467 of dupont merck; inosine; inosinic acid; inositol 1,3,4,5-tetrakisphosphate; inositol 1,3-bisphosphate; inositol-(1,3,4,5,6)-pentakisphosphate; insulin aspart; insulin beef; insulin degludec; insulin detemir; insulin glargine; insulin glulisine; insulin human; insulin lispro; insulin pork; interferon alfa-2a, recombinant; interferon alfa-2b, recombinant; interferon alfacon-1; interferon alfa-n1; interferon alfa-n3; interferon beta-1a; interferon beta-1b; interferon gamma-1b; intranasal apomorphine; intranasal morphine; inulin; invert sugar; iobenguane; iobenguane sulfate i-123; iodamide; iodine povacrylex; iodipamide; iodixanol; iodophenyl; iodo-willardiine; ioflupane 123; iohexol; Iopamidol; iopanoic acid; iophendylate; iopodic acid; iopromide; iothalamic acid; ioversol; ioxaglic acid; ioxilan; ipilimumab; ipratropium bromide; iproclozide; iproniazid; irbesartan; irinotecan; iron; iron dextran; iron sucrose; isatin; isatoic anhydride; isatoribine; isavuconazonium; isoaminile; isobutyric acid; isocarboxazid; isochorismic acid; isocitrate calcium complex; isocitric acid; isoconazole; isoetarine; isoflurane; isoflurophate; isoformononetin; isoluminol; isometheptene; isoniazid; isopenicillin n; isopentenyl pyrophosphate; isopentyl pyrophosphate; isoprenaline; isopropamide; isopropyl alcohol; isopropyl beta-d-thiogalactopyranoside; isoquinoline; isosorbide dinitrate; isosorbide mononitrate; isosulfan blue; isothiazolidinone analog; isothipendyl; isotretinoin; isovaleric acid; isoxicam; isoxsuprine; Isradipine; itopride; itraconazole; ivabradine; ivacaftor; ivermectin; ixabepilone; ixazomib; ixekizumab; jaspisamide a; josamycin; kabiramide c; kaempherol; kanamycin; kaolin; kappadione; kava; kebuzone; ketamine; ketazolam; ketobemidone; ketoconazole; ketoprofen; ketorolac; ketotifen; keyhole limpet hemocyanin; kifunensine; kitasamycin; l-[(n-hydroxyamino)carbonyl]phenylalanine; l-2-amino-3-butynoic acid; l-2-amino-4-(guanidinooxy)butyric acid; l-2-amino-4-[2-aminophenyl]-4-oxobutanoic acid; l-2-amino-4-methoxy-cis-but-3-enoic acid; l-2-amino-6-methylene-pimelic acid; labetalol; labetuzumab; lacidipine; lacosamide; lactaldehyde; lactic acid; lactose; lactose sialic acid; lactulose; laevulinic acid; l-alanine; l-alpha-glycerophosphorylserine; lamivudine; lamotrigine; lanosterol; lanreotide; lansoprazole; lanthanum carbonate; lapatinib; laquinimod; l-arginine; laronidase; l-asparagine; l-aspartic acid; latamoxef; latanoprost; latrunculin a; latrunculin b; lauric acid; lauryl dimethylamine-n-oxide; l-benzylsuccinic acid; l-carnitine; l-citrulline; l-cysteine; l-cystine; ledipasvir; leflunomide; lefradafiban; lemuteporfin; lenalidomide; lenvatinib; lepirudin; leptin; lerafaon; lercanidipine; lesinurad; lesopitron; letosteine; letrozole; leucine-reduced carbonyl; leucine phosphonic acid; leucovorin; leuprolide; levallorphan; levamisole; levamlodipine; levetiracetam; levobetaxolol; levobunolol; levobupivacaine; levocabastine; levocetirizine; levodopa; levofloxacin; levoleucovorin; levomethadyl acetate; levomethamphetamine; levomilnacipran; levonordefrin; levonorgestrel; levopropoxyphene; levorphanol; levosimendan; levothyroxine; l-glucuronic acid; l-glutamic acid; l-glutamine; l-glycero-d-manno-heptopyranose; l-guluronic acid 6-phosphate; l-histidine; l-histidine beta naphthylamide; l-homoarginine; l-homoserine; licofelone; lidocaine; lifitegrast; limaprost; linaclotide; linagliptin; lincomycin; lindane; linezolid; lintitript; liothyronine; liotrix; lipoic acid; liposomal prostaglandin el; liraglutide; lisdexamfetamine; lisinopril; l-iso-aspartate; l-isoleucine; lisuride; lithium; lividomycin a; lixisenatide; l-leucine; l-leucyl-hydroxylamine; l-lysine; l-methionine; l-methionine (r)-s-oxide; l-methionine (s)-s-oxide; l-myo-inositol-l-phosphate; lobeglitazone; lobeline; Iodoxamide; lofentanil; lofexidine; lomefloxacin; lomitapide; lomustine; loperamide; lopinavir; loracarbef; loratadine; lorazepam; lorcaserin; l-ornithine; lornoxicam; lorpiprazole; lortalamine; losartan; loteprednol; lovastatin; loxapine; loxoprofen; lpc-ether; l-phenylalanine; l-phenylalaninol; l-phospholactate; l-proline; l-rhamnitol; l-rhamnose; l-serine; l-thiocitrulline; l-thioproline; l-threo-2,3-diamino-butyric acid; l-threonine; I-threonohydroxamate 4-phosphate; l-tryptophan; l-tryptophanamide; l-tyrosinamide; l-tyrosine; lubazodone; lubiprostone; lucanthone; lucinactantluliconazole; lumacaftor; lumateperone; lumefantrine; lumiracoxib; lurasidone; lutropin alfa; l-valine; l-valinol; l-xylitol 5-phosphate; l-xylopyranose; l-xylulose 5-phosphate; ly231514 tetra glu; lymecycline; lymphotoxin beta receptor; lysergic acid diethylamide; lysine nz-carboxylic acid; lysophosphatidylglycerol; lysophosphotidylserine; macitentan; mafenide; magaldrate; magnesium; magnesium acetate tetrahydrate; magnesium carbonate; magnesium chloride; magnesium hydroxide; magnesium oxide; magnesium salicylate; magnesium sulfate; magnesium trisilicate; malachite green; malate ion; malate like intermediate; malathion; maleic acid; malonaldehyde; malonate ion; malonic acid; malonyl-coenzyme a; maltose; maltotetraose; m-aminophenylboronic acid; mangafodipir; manganese; manidipine; mannitol; mant-adp; maprotiline; maraviroc; marimastat; masoprocol; matairesinol; matuzumab; maxy-g34; mazindol; m-cresol; mebanazine; mebendazole; mecamylamine; mecasermin; mechlorethamine; meclizine; meclofenamic acid; medical air; medrogestone; medroxyprogesterone acetate; medrysone; mefenamic acid; mefloquine; megestrol acetate; melatonin; meloxicam; melperone; melphalan; memantine; menadione; menotropins; menthol; mepenzolate; mephentermine; mephenytoin; mepiprazole; mepivacaine; mepolizumab; meprobamate; mepyramine; mequitazine; mercaptocarboxylate inhibitor; mercaptomethyl phosphonate; mercaptopurine; mercuribenzoic acid; mercury acetate ion; mercury diiodide; merimepodib; meropenem; mersalyl; mesalazine; mesenchymal stem cells; mesna; mesobiliverdin iv alpha; meso-erythritol; mesoheme; mesoridazine; mestranol; meta vanadate; metamizole; metanitrophenyl-alpha-d-galactoside; meta-nitro-tyrosine; metaraminol; meta-tyrosine; metaxalone; metformin; methacholine; methacrylyl-coenzyme a; methacycline; methadone; methadyl acetate; methamphetamine; methantheline; methapyrilene; methaqualone; metharbital; methazolamide; methdilazine; methicillin acyl-serine; methimazole; methionine phosphinate; methionine phosphonate; methocarbamol; methohexital; methotrexate; methotrimeprazine; methoxamine; methoxsalen; methoxyflurane; methoxyundecylphosphinic acid; methsuximide; methyclothiazide; methyl alpha-d-mannoside; methyl alpha-galactoside; methyl aminolevulinate; methyl beta-galactoside; methyl cellulose; methyl isocyanide; methyl 1-phenylalaninate; methyl methylsulfinylmethyl sulfide; methyl nonanoate (ester); methyl salicylate; methylamine; methyl-carbamic acid ethyl ester; methylcobalamin; methyldiazene; methyldopa; methylene blue; methylenedioxyethamphetamine; methylergometrine; methylethylamine; methylmalonic acid; methylmalonyl-coenzyme a; methylnaltrexone; methyl-o3-(alpha-d-mannose)-alpha-d-mannose; methylphenidate; methylphenobarbital; methyl-phe-pro-amino-cyclohexylglycine; methylphosphinic acid; methylphosphonic acid diisopropyl ester; methylphosphonic acid ester group; methyl-phosphonic acid mono-(4-nitro-phenyl) ester; methylprednisolone; methylscopolamine bromide; methyltestosterone; methylthioinosine; methyltrienolone; methylumbelliferyl chitotriose; methylumbelliferyl sialic acid; methyprylon; methysergide; metiamide; meticillin; metipranolol; metixene; metoclopramide; metocurine; metocurine iodide; metolazone; metoprine, methodichlorophen; metoprolol; metralindole; metreleptin; metrizamide; metrizoic acid; metronidazole; metyrapone; metyrosine; mevastatin; mexiletine; mezlocillin; mianserin; mibefradil; micafungin; miconazole; microplasmin; midazolam; midodrine; mifepristone; migalastat; miglitol; miglustat; milnacipran; milrinone; miltefosine; mimosine; minaprine; minocycline; minoxidil; mipomersen; mirabegron; mirfentanil; mirtazapine; misoprostol; mitiglinide; mitomycin; mitoquinone; mitotane; mitoxantrone; mivacurium; mmda; moclobemide; modafinil; modified acarbose hexasaccharide; modified acarbose pentasaccharide; modified ribosylated glutamyl ester; moexipril; molecular iodine; molindone; molsidomine; molybdenum cofactor; mometasone; monastrol; monoazido-mu-oxo-diiron; monobenzone; monogalactosyl-diacylglycerol; monoisopropyl ester phosphonic acid group; monoisopropylphosphorylserine; monopotassium phosphate; monothioglycerol; montelukast; moricizine; morniflumate; morphine; motexafin gadolinium; motexafin lutetium; motuporin; moxalactam (hydrolyzed); moxalactam derivative; moxifloxacin; moxisylyte; moxonidine; mt-immucillin-h; mupirocin; muromonab; mycophenolate mofetil; mycophenolic acid; myo-inositol; myricetin; myristic acid; myristoyl-coa; myrrh; myxopyronin b; myxothiazol; n-(1-adamantyl)-n′-(4-guanidinobenzyl)urea; n,n-dimethylarginine; n,n-dimethyl-l-alanine; n,o6-disulfo-glucosamine; n,o-didansyl-l-tyrosine; n-[(4-methoxyphenyl)sulfonyl]-d-alanine; n-[(aminooxy)carbonyl]aniline; n-[4-(benzyloxy)phenyl]glycinamide; n-[amino(imino)methyl]glycine; n˜2˜succinylarginine; n˜2˜-succinylornithine; n˜3˜-benzylpyridine-2,3-diamine; n-1,10-phenanthrolin-5-ylacetamide; n-1-methylheptylformamide; n2-(carboxyethyl)-1-arginine; n3, n4-dimethylarginine; n5-(1-imino-3-butenyl)-1-ornithine; n5-iminoethyl-l-ornithine; n5-methylglutamine; n6-benzyl adenosine-5′-diphosphate; n7-methyl-formycin a; n7-methyl-guanosine-5′-monophosphate; nabilone; nabumetone; n-acetyl serotonin; n-acetylalanine; n-acetyl-alpha-d-glucosamine; n-acetyl-alpha-neuraminic acid; n-acetyl-beta-neuraminic acid; n-acetyl-d-allosamine; n-acetyl-d-galactosamine 6-sulfate; n-acetyl-d-glucosamine; n-acetylhistamine; n-acetyl-1-citrulline; n-acetyl-1-glutamate; n-acetyl-1-glutamine; n-acetylmannosaminitol; n-acetylmethionine; n-acetyl-n′-beta-d-glucopyranosyl urea; n-acetyl-p-nitrophenylserinol; n-acetylproline; n-acetyl-serine; nadh; nadolol; nadroparin; nafarelin; nafcillin; naftifine; nalbuphine; nalidixic acid; n-allyl-aniline; naloxegol; naloxone; n-alpha-acetyl-3,5-diiodotyrosylglycine; n-alpha-1-acetyl-arginine; naltrexone; naltrexone depot; nam napthylaminoalanine; n-aminoethylmorpholine; namn; nanaomycin d; nandrolone decanoate; nandrolone phenpropionate; naphazoline; naphthalen-1-yl-acetic acid; naphthalen-2-yl-3-alanine; naphthalene trisulfonate; naphthalene-1,2,4,5,7-pentol; naphthalene-1,2-diol; naphthalene-2,6-disulfonic acid; naphthyloxyacetic acid; naphthyridine inhibitor; naproxen; naratriptan; naringenin; natalizumab; natamycin; nateglinide; natural alpha interferon; naxifylline; n-benzoyl-d-alanine; n-benzoyl-n′-beta-d-glucopyranosyl urea; n-benzyl-4-[(2r)-pyrrolidin-2-ylmethoxy]aniline; n-benzyl-4-sulfamoyl-benzamide; n-benzylformamide; n-bromoacetyl-aminoethyl phosphate; n-butyl isocyanide; n-butyl-benzenesulfonamide; n-butyl-n′-hydroxyguanidine; n-carbamoyl-alanine; n-carbamoyl-l-aspartate; n-carbamyl-d-methionine; n-carbamyl-d-valine; n-carboxymethionine; n-cholylglycine; n-coeleneterazine; ncs-chromophore; n-cyclohexyl-n′-(4-iodophenyl)urea; n-cyclohexyl-n′-(propyl)phenyl urea; n-cyclohexyl-n′-decylurea; n-cyclohexyltaurine; n-cyclopentyl-n-cyclobutylformamide; nd1-phosphonohistidine; n-dimethyl-lysine; n-dodecanoyl-l-tyrosine; n-dodecyl-n,n-dimethylglycinate; neamine; nebivolol; necitumumab; nedocromil; nefazodone; nelarabine; nelfinavir; neocartilage; neomycin; neostigmine; nepafenac; neramexane; neridronic acid; nesiritide; n-ethyl retinamide; n-ethyl-5′-carboxamido adenosine; n-ethylmaleimide; netilmicin; netoglitazone; netupitant; nevirapine; n-formylmethionine; n-formylpiperidine; n-heptylformamide; n-hexadecanoylglycine; n-hexanoyl-l-homocysteine; n-hexanoyl-l-homoserine; n-hexylphosphonate ethyl ester; n-hydroxy-4-phosphono-butanamide; n-hydroxyguanidine; n-hydroxy-n-isopropyloxamic acid; niacin; nialamide; nicardipine; nicergoline; niclosamide; nicorandil; nicotinamide; nicotinamide mononucleotide; nicotine; nifedipine; niflumic acid; niguldipine; nilotinib; niludipine; nilutamide; nilvadipine; nimesulide; nimodipine; nintedanib; nisoldipine; n-isopropyl-n′-hydroxyguanidine; nisoxetine; nitazoxanide; nitisinone; nitrazepam; nitrendipine; nitric oxide; nitrilotriacetic acid; nitroarginine; nitrocefin; nitrocefin acyl-serine; nitrofural; nitrofurantoin; nitrogen; nitroglycerin; nitromethyldethia coenzyme a; nitroprusside; nitrosoethane; nitrous oxide; nitroxoline; nivolumab; nizatidine; n′-l-seryl-3′-amino-(3′-deoxy)-adenosine; n-methyl-alpha-beta-dehydroalanine; n-methyldehydrobutyrine; n-methylleucine; n-methyl-lysine; n-methylmesoporphyrin; n-methylmesoporphyrin containing copper; n-methyl-n-(methylbenzyl)formamide; n-methylnaloxonium; n-methyl-n-propargyl-1(r)-aminoindan; n-methyl-pyridoxal-5′-phosphate; nocodazole; n-octyl-2-hydroxyethyl sulfoxide; nogalaviketone; nojirimycine tetrazole; n-omega-hydroxy-l-arginine; n-omega-propyl-1-arginine; nomifensine; nonan-1-ol; nonoxynol-9; norcamphor; norelgestromin; norepinephrine; norethisterone; norfloxacin; norgestimate; norgestrel; norleucine; norleucine phosphonate; normethadone; nor-n-omega-hydroxy-l-arginine; nortriptyline; norvaline; novo nordisk a/s compound; novobiocin; n-oxo-2-(phenylsulfonylamino)ethanamide; n-phenyl-1h-pyrazole-3-carboxamide; n-phenyl-1h-pyrrolo[2,3-b]pyridin-3-amine; n-phenylthiourea; n-propargyl-1(s)-aminoindan; n-propyl isocyanide; n-propyl-tartramic acid; n-pyridoxyl-2-methylalanine-5-phosphate; n-pyridoxyl-glycine-5-monophosphate; n′-pyridoxyl-lysine-5′-monophosphate; n-pyridoxyl-threonine-5-monophosphate; n-succinyl methionine; n-succinyl phenylglycine; n-sulfo-flavin mononucleotide; n-tridecanoic acid; n-trimethyllysine; n-valeric acid; nvs antibody; nystatin; nz-(1-carboxyethyl)-lysine; nz-(dicarboxymethyl)lysine; nz2-tryptophan; o1-methyl-4-deoxy-4-thio-alpha-d-glucose; o1-methyl-4-deoxy-4-thio-beta-d-glucose; o1-methyl-glucose; o1-pentyl-mannose; o2-sulfo-glucuronic acid; o3-sulfonylgalactose; o4-sulfonylgalactose; o-acetyl-1-serine; o-benzylsulfonyl-serine; obeticholic acid; obiltoxaximab; obinutuzumab; ocriplasmin; octahydroindole-2-carboxylic acid; octamethylenediamine; octamoxin; octane-1,3,5,7-tetracarboxylic acid; octanoyl-coenzyme a; octinoxate; octisalate; octocrylene; octreotide; octyl alpha-l-altropyranoside; octyl beta-d-galactopyranoside; octylphenoxy polyethoxyethanol; o-decyl hydrogen thiocarbonate; ofatumumab; ofloxacin; oglufanide disodium; ohmefentanyl; olanzapine; olaparib; olaratumab; olcegepant; oleic acid; oleoyl estrone; olive oil; olmesartan; olodaterol; olomoucine; olomoucine ii; olopatadine; olsalazine; omacetaxine mepesuccinate; omalizumab; omapatrilat; ombitasvir; omega interferon; omega-3-acid ethyl esters; omega-3-carboxylic acids; omeprazole; ondansetron; o-phosphoethanolamine; oportuzumab monatox; oprelvekin; orciprenaline; oregon green 488 carboxylate; oregovomab; oritavancin; orlistat; orotic acid; orotidine-5-monophosphate; orphenadrine; osanetant; oseltamivir; osimertinib; ospa lipoprotein; ospemifene; o-succinylbenzoate; o-sulfo-l-serine; oteracil; o-trifluoromethylphenyl anthranilic acid; ouabain; ovalicin; oxacillin; oxalic acid; oxaliplatin; oxaloacetate ion; oxamic acid; oxamniquine; oxandrolone; oxaprotiline; oxaprozin; oxazepam; oxcarbazepine; oxeladin; oxfenicine; oxibendazole; oxiconazole; oxidized acetyl dithranol; oxidized coenzyme a; oxidized glutathione disulfide; oxilofrine; oximinoarylsulfonamide; oxiranpseudoglucose; oxitriptan; oxprenolol; oxtriphylline; oxybenzone; oxybuprocaine; oxybutynin; oxycodone; oxygen; oxymetazoline; oxymetholone; oxymorphone; oxyphenbutazone; oxyphencyclimine; oxyphenisatin; oxyphenonium; oxypurinol; oxytetracycline; oxytocin; paclitaxel; pagoclone; palbociclib; palifermin; paliperidone; palivizumab; palmitic acid; palmitoleic acid; palmitoyl-linoleoyl phosphatidylcholine; palonosetron; pamidronate; p-aminophenyl-alpha-d-galactopyranoside; pancrelipase; pancuronium; p-anisic acid; panitumumab; panobinostat; pantoprazole; pantothenic acid; pantothenoylaminoethenethiol; pantoyl adenylate; papaverine; para-(benzoyl)-phenylalanine; para-bromobenzyl alcohol; para-coumaric acid; para-iodo-d-phenylalanine hydroxamic acid; para-isopropylaniline; paraldehyde; para-mercury-benzenesulfonic acid; paramethadione; paramethasone; parathyroid hormone; para-toluene sulfonate; parecoxib; pargyline; paricalcitol; paritaprevir; parnaparin; paromomycin; paroxetine; pasireotide; patiromer; pazopanib; pcnotaxime group; p-cresol; pefloxacin; pegademase bovine; pegaptanib; pegaspargase; pegfilgrastim; peginesatide; peg-infergen; peginterferon alfa-2a; peginterferon alfa-2b; peginterferon beta-1a; [pterin-6-ylmethanyl]-phosphonophosphate; pegloticase; peg-uricase; pegvisomant; peldesine; pembrolizumab; pemetrexed; pemirolast; pemoline; penbutolol; penciclovir; penicillamine; penicillin g acyl-serine; pentabromophenol; pentagastrin; pentamidine; pentanal; pentane-1,5-diamine; pentanedial; pentastarch; pentasulfide-sulfur; pentazocine; pentobarbital; pentolinium; pentosan polysulfate; pentostatin; pentoxifylline; pentyl trihydrogen diphosphate; peramivir; perampanel; perchlorate ion; perflubron emulsion; perflutren; pergolide; perhexiline; peridinin; perindopril; permethrin; perospirone; perphenazine; persumac; pertuzumab; pethidine; petrolatum; pexelizumab; phenacemide; phenacetin; phenaridine; phenazopyridine; phencyclidine; phendimetrazine; phenelzine; phenformin; phenindamine; phenindione; pheniprazine; pheniramine; phenmetrazine; phenobarbital; phenol; phenolphthalein; phenoxodiol; phenoxybenzamine; phenoxymethylpenicillin; phenoxypropazine; phenprocoumon; phenserine; phensuximide; phentermine; phentolamine; phenyl boronic acid; phenyl ethenesulfonate; phenylacetaldehyde; phenylacetic acid; phenylalanindiol; phenylalanine amide; phenylalanine boronic acid; phenylalanine-n-sulfonamide; phenylalanylmethane; phenylalanylmethylchloride; phenylaminoimidazo(1,2-alpha)pyridine; phenylbutazone; phenyldehydroalanine; phenylephrine; phenylethane boronic acid; phenylferricrocin-iron; phenylphosphate; phenylpropanolamine; phenyl-uridine-5′-diphosphate; phenytoin; pholcodine; phosphatidyl ethanol; phosphatidyl serine; phosphatidylethanolamine; phosphoaspartate; phosphocholine; phosphoenolpyruvate; phosphoglycolohydroxamic acid; phosphonoacetaldehyde; phosphonoacetic acid; phosphonoacetohydroxamic acid; phosphonopyruvate; phosphonoserine; phosphonothreonine; phosphonotyrosine; phosphoramidon; phosphoribosyl atp; phosphoric acid; phosphorylated dihydropteroate; phosphorylisopropane; phthalic acid; p-hydroxybenzaldehyde; p-hydroxybenzoic acid; phylloquinone; physostigmine; piboserod; piceatannol; piclamilast; picoplatin; picosulfuric acid; picrotoxin; pidotimod; pilocarpine; pimagedine hcl; pimavanserin; pimecrolimus; pimelic acid; pimozide; pinaverium; pindolol; pioglitazone; pipamperone; pipazethate; pipecuronium; piperacillin; piperazine; piperonyl butoxide; pipobroman; pipotiazine; piracetam; pirarubicin; pirbuterol; pirenzepine; piretanide; pirfenidone; pirlindole; piroxicam; pitavastatin; pivaloyloxymethyl butyrate; pivampicillin; pivhydrazine; pivmecillinam; pixantrone; plasmin; platelet activating factor; platensimycin; pleconaril; plerixafor; plicamycin; plitidepsin; p-nitrophenol; podofilox; podophyllin; polaprezinc; polidocanol; polyacrylic acid; polycarbophil calcium; polyethylene glycol; polymyxin b sulfate; polystyrene sulfonate; polythiazide; pomalidomide; ponatinib; poractant alfa; porfimer; porphobilinogen; porphyrin fe(iii); posaconazole; posizolid; potassium; potassium alum; potassium chloride; potassium citrate; potassium iodide; povidone-iodine; practolol; pradefovir mesylate; pralatrexate; pralidoxime; pralnacasan; pramipexole; pramlintide; pramocaine; pranlukast; prasugrel; pravastatin; prazepam; praziquantel; prazosin; prednicarbate; prednisolone; prednisone; pregabalin; pregnenolone; prenylamine; prephenic acid; prilocaine; primaquine; primidone; prinomastat; probenecid; probucol; procainamide; procaine; procaine benzylpenicillin; procarbazine; procaterol; prochlorperazine; procyclidine; proellex; proflavine; progabide; progesterone; proguanil; protease-r-hfsh; proline betaine; promazine; promethazine; propacetamol; propafenone; propanoic acid; propantheline; proparacaine; propericiazine; propidium; propiomazine; propionamide; propionyl coenzyme a; propofol; propoxycaine; propoxyphene napsylate; propranolol; propyl acetate; propyl trihydrogen diphosphate; propyl-1-phosphate; propylhexedrine; propylthiouracil; prostaglandin b2; prostaglandin g2; protamine sulfate; protirelin; protoporphyrin ix; protoporphyrin ix containing co; protoporphyrin ix containing zn; protriptyline; prucalopride; prussian blue; pseudoephedrine; pseudotropine; pseudouridine-5′-monophosphate; pteric acid; pterin cytosine dinucleotide; pterin-6-yl-methyl-monophosphate; pteroic acid; pumactant; purine riboside; purine riboside-5′-monophosphate; puromycin; purvalanol; purvalanol a; putrescine; pyoverdine-chromophore; pyrazinamide; pyrazole; pyridin-3-ylmethanol; pyridostigmine; pyridoxal; pyridoxal phosphate; pyridoxal-5′-phosphate-n-oxide; pyridoxamine-5′-phosphate; pyridoxine; pyridoxine-5′-phosphate; pyridoxyl-alanine-5-phosphate; pyridoxyl-glutamic monophosphate; pyrimethamine; pyrithiamine pyrophosphate; pyroglutamate; pyroglutamic acid; pyromellitic acid; pyrrole-2-carboxylate; pyrroloquinoline quinone; pyruvamide; pyruvate; pyruvic acid; pyruvoyl group; pyrvinium; quazepam; quercetin; quetiapine; quinacrine; quinagolide; quinaldic acid; quinapril; quinestrol; quinethazone; quinidine; quinidine barbiturate; quinine; quinolinic acid; quinonoid 7,8-tetrahydrobiopterin; quinupristin; quisqualate; r,3-hydroxybutan-2-one; rabeprazole; radicicol; radium ra 223 dichloride; ragweed pollen extract; raloxifene; raloxifene core; raltegravir; raltitrexed; ramelteon; ramipril; ramoplanin; ramosetron; ramucirumab; ranibizumab; ranitidine; ranolazine; ranpirnase; rapacuronium; rasagiline; rasburicase; raxibacumab; r-azabisabolene; rbt205 inhibitor; reactive red 1 dye; reboxetine; recombinant alpha 1-antitrypsin; recombinant human gm-csf; recombinant human relaxin; reduced threonine; regadenoson; reglixane; regorafenib; reidispongiolide a; reidispongiolide c; remifentanil; remikiren; remoxipride; renzapride; repaglinide; rescinnamine; reserpine; resveratrol; retapamulin; reteplase; reviparin; rhodamine 6g; ribavirin; riboflavin; riboflavin monophosphate; ribose; ribose-1-phosphate; ribose-5-phosphate; ribostamycin; ribulose-5-phosphate; ricinoleic acid; ridogrel; rifabutin; rifalazil; rifampicin; rifamycin cgp 4832; rifapentine; rifaximin; rilapladib; rilonacept; rilpivirine; riluzole; rimantadine; rimexolone; rimonabant; riociguat; risedronate; risperidone; ritodrine; ritonavir; rituximab; rivanicline; rivaroxaban; rivastigmine; rivoglitazone; rizatriptan; rocuronium; rofecoxib; roflumilast; rolapitant; rolicyclidine; rolipram; rolitetracycline; romidepsin; romiplostim; ropinirole; ropivacaine; roquinimex; rosiglitazone; rosoxacin; rosuvastatin; rotigotine; roxatidine acetate; roxithromycin; r-styrene oxide; rubidium chloride rb-82; rufinamide; rupatadine; rutin; ruxolitinib; s-(2-oxo)pentadecylcoa; s-(3-iodobenzyl)glutathione; s-(4-bromobenzyl)cysteine; s-(d-carboxybutyl)-1-homocysteine; s-(dimethylarsenic)cysteine; s-(methylmercury)-1-cysteine; s-(p-nitrobenzyl)glutathione; s,3-hydroxybutan-2-one; s,s-(2-hydroxyethyl)thiocysteine; s,s-propylthiocysteine; s-1,2-propanediol; s-2-(boronoethyl)-1-cysteine; s-4-nitrobutyryl-coa; s-acetonylcysteine; s-acetyl-cysteine; sacrosidase; sacubitril; s-adenosyl-1,8-diamino-3-thiooctane; s-adenosyl-1-homocysteine; s-adenosyl-l-homoselenocysteine; s-adenosylmethionine; safrazine; salbutamol; salicylamide; salicylhydroxamic acid; salicylic acid; salmeterol; salmon calcitonin; salophen iron chelate; salophen-10-carboxylate iron chelate; salophen-10-propionate iron chelate; salsalate; samarium (153sm)lexidronam; samarium sm-153 lexidronam pentasodium; saprisartan; sapropterin; saquinavir; sar9, met (02)11-substance p; sarafloxacin; sargramostim; s-arsonocysteine; satraplatin; s-atrolactic acid; satumomab pendetide; saxagliptin; s-azabisabolene; s-benzyl-glutathione; s-butyryl-cystein; scopolamine; scopolamine butylbromide; s-dioxymethionine; sebacic acid; sebelipase alfa; secobarbital; secretin; secukinumab; se-ethyl-isoselenourea; selegiline; selenium sulfide; selenocysteine; selenoinosine; selenomethionine selenoxide; selexipag; senna glycoside; seocalcitol; seproxetine; serine vanadate; sermorelin; sertaconazole; sertindole; sertraline; serum albumin; serum albumin iodonated; s-ethylisothiourea; s-ethyl-n-phenyl-isothiourea; setiptiline; sevelamer; sevoflurane; s-hexylglutathione; shikimate-3-phosphate; s-hydroxycysteine; s-hydroxymethyl glutathione; sibutramine; sildenafil; silibinin a; silodosin; siltuximab; silver sulfadiazine; simeprevir; simethicone; simoctocog alfa; simvastatin; sinapinate; sinapoyl coenzyme a; sincalide; sinecatechins; sinefungin; sipuleucel-t; siroheme; sirolimus; s-isopropyl-isothiourea; sitagliptin; sitamaquine; sitaxentan; skeletal targeted radiotherapy; s-mercaptocysteine; s-methyl phosphocysteine; s-methyl thiocysteine group; s-methylcysteine; s-methyl-glutathione; s-nonyl-cysteine; s-octylglutathione; sodium acetate; sodium aurothiomalate; sodium bicarbonate; sodium chloride; sodium citrate; sodium ferric gluconate complex; sodium fluoride; sodium fluoride f-18; sodium gluconate; sodium glycerophosphate; sodium iodide i-123; sodium iodide i-131; sodium lauryl sulfate; sodium monofluorophosphate; sodium nitrite; sodium oxybate; sodium phenylbutyrate; sodium phosphate; sodium stibogluconate; sodium sulfate; sodium tetradecyl sulfate; sodium thiosulfate; sofosbuvir; solabegron; solifenacin; solithromycin; somatostatin; somatrem; somatropin recombinant; sonidegib; sorafenib; soraphen a; sorbinil; sorbitol; sotalol; s-oxy cysteine; soybean spaglumic acid; s-palmitoyl-l-cysteine; sparfloxacin; sparfosic acid; sparsomycin; sparteine; spectinomycin; spermidine; spermine; sphingosine; sphinxolide b; s-phosphocysteine; spinosad; spirapril; spironolactone; s-p-nitrobenzyloxycarbonylglutathione; s-propylamine-l-cysteine; s-selanyl cysteine; s-sulphocysteine; st. john's wort; stannous fluoride; stannsoporfin; stanozolol; staurosporine; stavudine; stearic acid; stepronin; stiripentol; streptokinase; streptolydigin; streptomycin; streptozocin; strontium chloride sr-89; strontium malonate; strontium ranelate; succimer; succinamide-coa; succinic acid; succinylcholine; succinyl-coenzyme a; sucralfate; sucroferric oxyhydroxide; sucrose; sucrosofate; sufentanil; sugammadex; sulbactam; sulconazole; sulfacetamide; sulfacytine; sulfadiazine; sulfadimethoxine; sulfadoxine; sulfamerazine; sulfamethazine; sulfamethizole; sulfamethoxazole; sulfametopyrazine; sulfamoxole; sulfanilamide; sulfaphenazole; sulfapyridine; sulfasalazine; sulfathiazole; sulfatide; sulfinpyrazone; sulfisoxazole; sulfopyruvate; sulfoxone; sulfur; sulfur hexafluoride; sulindac; sulodexide; sulpiride; sulthiame; sumatriptan; sunitinib; suprofen; suramin; susoctocog alfa; suvorexant; swainsonine; synephrine; synthetic conjugated estrogens, a; synthetic conjugated estrogens, b; syringate; tacrine; tacrine(8)-4-aminoquinoline; tacrolimus; tadalafil; tafluprost; tagatose; tagetitoxin; talactoferrin alpha; talampanel; talbutal; talc; taliglucerase alfa; talniflumate; talopram; talsupram; tamibarotene; tamoxifen; tamsulosin; tanaproget; tandamine; tannic acid; tapentadol; tartaric acid; tartronate; tasimelteon; tasosartan; tasquinimod; taurocholic acid; tauroursodeoxycholic acid; tavaborole; tazarotene; tazobactam; tazobactam intermediate; tecadenoson; technetium tc 99m mebrofenin; technetium tc 99m oxidronate; technetium tc 99m sulfur colloid; technetium tc-99m disofenin; technetium tc-99m exametazime; technetium tc-99m medronate; technetium tc-99m pyrophosphate; technetium tc-99m sestamibi; technetium tc-99m sodium pertechnetate; technetium tc-99m tetrofosmin; technetium tc-99m tilmanocept; tedizolid phosphate; teduglutide; tegafur; tegafur-uracil; tegaserod; teicoplanin; telaprevir; telavancin; telbivudine; telithromycin; telmisartan; temafloxacin; temazepam; temocapril; temozolomide; temsirolimus; tenecteplase; teniposide; tenocyclidine; tenofovir; tenofovir alafenamide; tenoxicam; terazosin; terbinafine; terbutaline; terconazole; terfenadine; teriflunomide; teriparatide; terlipressin; tert-butyloxycarbonyl group; tesamorelin; tesmilifene; testolactone; testosterone; testosterone hemisuccinate; testosterone propionate; tetra(imidazole)diaquacopper (i); tetra(imidazole)diaquacopper (0; tetrabenazine; tetrabromo-2-benzotriazole; tetrabutylammonium ion; tetracaine; tetracycline; tetraethylammonium; tetrafluoroaluminate ion; tetrahydrodeoxyuridine; tetrahydrofolic acid; tetrahydrofuran-2-carboxylic acid; tetrahydrooxazine; tetrahydropyran; tetramethylammonium ion; tetrathiomolybdate; tetrazolyl histidine; tetrodotoxin; tetryzoline; thalidomide; thallous chloride; thenalidine; thenoyltrifluoroacetone; theobromine; theophylline; thiabendazole; thiamin diphosphate; thiamin phosphate; thiamine; thiamphenicol; thiamylal; thiarsa dihydroxy cysteine; thiarsahydroxy-cysteine; thieno[2,3-b]pyridine-2-carboxamidine; thienylfentanyl; thiethylperazine; thimerosal; thio-atpa; thiocamphor; thiocellobiose; thiocolchicoside; thiocoumarin; thiodigalactoside; thio-maltohexaose; thio-maltopentaose; thionicotinamide-adenine-dinucleotide; thiopental; thioproperazine; thiopyrophosphate; thioridazine; thiorphan; thiotepa; thiothixene; thonzonium bromide; threonine derivative; threonine-aspartic ester; thymalfasin; thymidine-3′,5′-diphosphate; thymidine-5′-diphosphate; thymidine-5′-(dithio)phosphate; thymidine-5′-diphospho-beta-d-xylose; thymidine-5′-phosphate; thymidine-5′-thiophosphate; thymidine-5′-triphosphate; thymine; thymol; thyroglobulin; thyroid, porcine; thyrotropin alfa; tiagabine; tianeptine; tiaprofenic acid; tibolone; ticagrelor; ticarcillin; ticlopidine; ticrynafen; tigecycline; tiludronate; timolol; tinidazole; tinzaparin; tioconazole; tioguanine; tiopronin; tiotropium; tipifarnib; tipiracil; tipranavir; tirapazamine; tirofiban; titanium dioxide; tixocortol; tizanidine; tobramycin; tocainide; tocilizumab; tofacitinib; tofisopam; tolazamide; tolazoline; tolbutamide; tolcapone; tolfenamic acid; tolmetin; tolnaftate; toloxatone; tolrestat; tolterodine; toluene; tolvaptan; topiramate; topotecan; torasemide; toremifene; tositumomab; tosyl-d-proline; trabectedin; tramadol; trametinib; trandolapril; tranexamic acid; tranilast; trans-2-hydroxycinnamic acid; trans-2-phenylcyclopropylamine; trans-o-hydroxy-alpha-methyl cinnamate; trans-urocanic acid; tranylcypromine; trapidil; trastuzumab; trastuzumab emtansine; travoprost; trazeolide; trazodone; trefentanil; trehalose-6-phosphate; trencam-3,2-hopo; treprostinil; tretinoin; triamcinolone; triamterene; triazolam; triazolopyridine; triazolopyrimidine; tribenuron methyl; tribromomethane; tributylstannanyl; tricarballylic acid; trichlormethiazide; tri-chloro-acetaldehyde; triclosan; tricosanoic acid; tricyclazole; tridihexethyl; triethyl phosphate; triethylenetetramine; trifluoperazine; trifluoroacetonyl coenzyme a; trifluoroalanine; trifluoroethanol; trifluorofurnesyl diphosphate; trifluoromethionine; trifluoro-thiamin phosphate; triflupromazine; trifluridine; triflusal; triglu-5-formyl-tetrahydrofolate; trihexyphenidyl; trihydroxyantimonite(iii); trihydroxyarsenite(iii); trilostane; trimazosin; trimebutine; trimetazidine; trimethadione; trimethaphan; trimethobenzamide; trimethoprim; trimethyl glycine; trimetrexate; trimipramine; trinitrotoluene; trioxsalen; tripelennamine; triphospate; triprolidine; triptorelin; tris; tris(hydroxyethyl)aminomethane; trisalicylate-choline; tris-hydroxymethyl-methyl-ammonium; trisodium citrate dihydrate; trivanadate; troglitazone; troleandomycin; tropicamide; tropinone; trospium; trovafloxacin; troxacitabine; trw3-(2-amino-3-hydroxy-propyl)-6-(n′-cyclohexyl-hydrazino)octahydro-indol-7-ol; trypan blue; trypanothione; tryptophanyl-5′amp; tubercidin; tuberculin purified protein derivative; tubocurarine; tungstopterin cofactor; turoctocog alfa; turpentine; tyloxapol; tymazoline; tyramine; tyropanoic acid; tyrosinal; tyrosyladenylate; tyvelose; ubenimex; ubiquinone-1; ubiquinone-2; udenafil; udp-alpha-d-glucuronic acid; udp-alpha-d-xylopyranose; ulapualide a; ularitide; ulipristal; ulobetasol; umeclidinium; undecan-2-one; undecanal; undecylamine-n,n-dimethyl-n-oxide; undecyl-beta-d-maltopyranoside; undecyl-phosphinic acid butyl ester; unoprostone; uracil; uracil arabinose-3′-phosphate; uracil mustard; urea; urea c-13; urea c-14; uric acid; uridine; uridine 5-triphosphate; uridine diphosphate glucose; uridine triacetate; uridine-2′,3-vanadate; uridine-5′-diphosphate; uridine-5′-diphosphate-mannose; uridine-5′-monophosphate; uridine-diphosphate-n-acetylgalactosamine; uridine-diphosphate-n-acetylglucosamine; uriclyl-2′-5′-phospho-adenosine; urofollitropin; urokinase; ursodeoxycholic acid; ustekinumab; valaciclovir; valdecoxib; valganciclovir; valproic acid; valpromide; valrubicin; valsartan; vancomycin; vandetanib; vanillate; vanoxerine; vapitadine dihydrochloride; vapreotide; vardenafil; varenicline; vasopressin; vatalanib; vayarin; vecuronium; vedolizumab; velaglucerase alfa; veliparib; velpatasvir; vemurafenib; venetoclax; venlafaxine; verapamil; verdoheme; verteporfin; vibriolysin; vidarabine; vigabatrin; vilanterol; vilazodone; vildagliptin; viloxazine; vinblastine; vincristine; vindesine; vinorelbine; vinylglycine; vinylsulphonic acid; violaxanthin; viomycin; virginiamycin m1; virginiamycin sl; vismodegib; vitamin a; vitamin c; vitamin e; voacamine; voglibose; vorapaxar; voriconazole; vorinostat; vortioxetine; warfarin; water; willardiine; xanthine; xanthinol; xanthophyll; xenon-133; ximelagatran; xylarohydroxamate; xylometazoline; xylose-derived imidazole; xylose-derived lactam oxime; yohimbine; zafirlukast; z-ala prolinal; zalcitabine; zaleplon; zaltoprofen; zanamivir; zanapezil; z-dehydrobutyrine; zebularine; ziconotide; zidovudine; zileuton; zimelidine; zinc; zinc oxide; zinc substituted heme c; zinc sulfate; zinc trihydroxide; ziprasidone; zk-epothilone; zn(ii)-(2r)-oxo-protoporphyrin ix); zoledronic acid; zolmitriptan; zolpidem; zomepirac; zonisamide; zopiclone; zorubicin; zotepine; z-pro-prolinal; zucapsaicin; zuclopenthixol; (10e,12z)-octadeca-10,12-dienoic acid; (10r)-10-formyl-5,8,10-trideazafolic acid; (10s)-10-formyl-5,8,10-trideazafolic acid; (1r)-2-amino-1-[3-(trifluoromethyl)phenyl]ethanol; chloro-1-phenylpropan-1-ol; (1r,45)-2-azabornane; (1s,2s)-1-amino-1-(1,3-thiazol-2-yl)propan-2-ol; (1s,3r,4r,6s)-1,3,4,6-tetrapkisphosphate; (1s,3s,4s)-1,3,4-triphospho-myo-inositol; (2,6-dimethyl-phenoxy)-acetic acid; (2-[2-ketopropylthio]ethanesulfonate; (2r)-1-(2,6-dimethylphenoxy)propan-2-amine; (2r)-2,3-dihydroxypropanal; (2r)-2-benzyl-3-nitropropanoic acid; (2s)-2-(1h-indol-3-yl)hexanoic acid; (2s)-2-(1h-indol-3-yl)pentanoic acid; (2s)-2-(4-chlorophenoxy)-3-phenylpropanoic acid; (2s)-2-(4-ethylphenoxy)-3-phenylpropanoic acid; (2s)-2-(biphenyl-4-yloxy)-3-phenylpropanoic acid; (2s)-2,8-diaminooctanoic acid; (2s)-2-hydroxyoctanoic acid; (2s)-hydroxy(4-hydroxyphenyl)ethanenitrile; (2s)-hydroxy(4-hydroxyphenyl)ethanoic acid; (2s)-pyrrolidin-2-ylmethylamine; (2s,3r)-1-amino-2-methylbutane-2,3-diol; (2s,3r)-3-(6-amino-9h-purin-9-yl)nonan-2-ol; (2s,3s)-2-azanyl-3-methyl-pentanedioic acid; (2s,3s)-trans-dihydroquercetin; (2s,4s)-alpha-campholinic acid; (2s,5s)-5-carboxymethylproline; (2-sulfanyl-3-phenylpropanoyl)-phe-tyr; (3beta)-cholest-5-ene-3,25-diol; (3beta,20r)-cholest-5-ene-3,20-diol; (3beta,7beta)-cholest-5-ene-3,7-diol; (3-chloro-4-propoxy-phenyl)-acetic acid; (3e)-2,6-dioxo-6-phenylhex-3-enoate; 3[n-morpholino]propane sulfonic acid; 3-isobutyl-1-methyl-7h-xanthine; 3-mercuri-4-aminobenzenesulfonamide; 3-(oxalyl-amino)-naphthalene-2-carboxylic acid; 3-(phosphonomethyl)pyridine-2-carboxylic acid; 3-(prop-2-ene-1-sulfinyl)-propene-1-thiol; 3,4-dihydro-2h-pyrrolium-5-carboxylate; 3,4-methylenedioxyamphetamine; 3,4-methylenedioxymethamphetamine; 3,4-epoxybutyl-alpha-d-glucopyranoside; 3,5,3′,5′-tetrachloro-biphenyl-4,4′-diol; 3,4-dihydro-5-methyl-isoquinolinone; 3,4-dihydroxy-1-methylquinolin-2(1h)-one; 3(s)-amino-4-phenyl-butan-2(r)-ol; 3(s)-amino-4-phenyl-butan-2(s)-ol; 3′,5′-dinitro-n-acetyl-1-thyronine; 3,6,9,12,15,18-hexaoxaicosane; 3,6,9,12,15-pentaoxaheptadecan-1-ol; 3,6,9,12,15-pentaoxatricosan-1-ol; 3,6-anhydro-d-galactose-2-sulfate; 3,6-dihydroxy-xanthene-9-propionic acid; 3,7,11,15-tetramethyl-hexadecan-1-ol; 3,7,3,4′-tetrahydroxyflavone; 3,5-difluorobenzenesulfonamide; 3-[(1s)-1-(dimethylamino)ethyl]phenol; (3-formyl-but-3-enyl)-phosphonic acid; (3r)-1-acetyl-3-methylpiperidine; (3r)-3-hydroxydodecanoic acid; (3s)-2,3,4,5-tetrahydropyridin-3-amine; (4e)-4-aminohex-4-enoic acid; (4-ethylphenyl)sulfamic acid; (4-fluorophenyl)(pyridin-4-yl)methanone; (4-hydroxymaltosephenyl)glycine; (4r)-2-methylpentane-2,4-diol; (4r)-7aza-7,8-dihydrolimonene; (4s)-5-fluoro-1-leucine; (4s,5s)-1,2-dithiane-4,5-diol; (6r)-folinic acid; (6s)-5,6,7,8-tetrahydrofolate; (8ar)-hexahydropyrrolo[1,2-a]pyrazine-1,4-dione; (c8-r)-hydantocidin 5′-phosphate; (c8-s)-hydantocidin 5′-phosphate; (carboxyhydroxyamino)ethanoic acid; (e)-2-fluoro-p-hydroxycinnamate; (e)-imidacloprid; (hydroxyethyloxy)tri(ethyloxy)octane; (molybdopterin-s,s)-dioxo-thio-molybdenum(v); (mu-4-sulfido)-tetra-nuclear copper ion; (p-iodophenylacetylamino)methylphosphinic acid; (r)-1-para-nitro-phenyl-2-azido-ethanol; (r)-1-phenylethanol; (r)-2-hydroxy-3-sulfopropanoic acid; (r)-4-nitrostyrene oxide; (r)-mandelic acid; (r)-mesopram; (r)-mevalonate; (r)-n-(1-methyl-hexyl)-formamide; (r)-n-(3-indol-1-yl-2-methyl-propyl)-4-sulfamoyl-benzamide; (r)-propylene glycol; (r)-pyridin-4-yl[4-(2-pyrrolidin-1-ylethoxy)phenyl]methanol; (r)-rolipram; (r)-tacrine(10)-hupyridone; (r,r)-2,3-butanediol; (s)-4-nitrostyrene oxide; (s)-atpa; (s)-blebbistatin; (s)-des-me-ampa; (s)-hmg-coa; (s)-hydroxy(phenyl)acetonitrile; (s)-mandelic acid; (s)-rolipram; (s)-tacrine(10)-hupyridone; (s)-wiskostatin; (s,r)-fidarestat; (south)-methanocarba-thymidine; (tert-butyloxycarbonyl)-alanyl-alanyl-amine; (z)-2-[2-(4-methylpiperazin-1-yl)benzyl]diazenecarbothioamide; [[4-(aminomethyl)phenyl]amino]oxo-acetic acid; [[n-(benzyloxycarbonyl)amino]methyl]phosphate; [2(formyl-hydroxy-amino)-ethyl]-phosphonic acid; [2(r,$)-2-sulfanylheptanoyl]-phe-ala; [methylseleno]acetate; [methyltelluro]acetate; [methylthio]acetate; 1-(1-phenylcyclopentyl)methylamine; 1-(2,2′-bithiophen-5-yl)methanamine; 1-(2-nitrophenyl)-2,2,2-trifluoroethyl]-arsenocholine; 1-(2-amidinophenyl)-3-(phenoxyphenyl)urea; 1-(4-amidinophenyl)-3-(4-chlorophenyl)urea; 1-(4-hexylphenyl)prop-2-en-1-one; 1-(4-thiophen-2-ylphenyl)methanamine; 1-(9-ethyl-9h-carbazol-3-yl)-n-methylmethanamine; 1-(adamantan-1-yl)-2-(1h-imidazol-1-yl)ethanone; 1-(biphenyl-4-ylmethyl)-1h-imidazole; 1,3,5-benzenetricarboxylic acid; 1,3-dedimethyl-1,3-divinyl heme; 1,4-deoxy-1,4-dithio-beta-d-glucopyranose; 1,4-dideoxy-o2-sulfo-glucuronic acid; 1-[4-(2-oxo-2-phenylethyl)phenyl]guanidine; 1-[4-(hydroxymethyl)phenyl]guanidine; 1-(2-phenylethyl)-4-phenyl-4-acetoxypiperidine; 1,10-phenanthroline; 1,1′-biphenyl-2-sulfinic acid; 1,1′-biphenyl-3,4-diol; 1,1′-hexane-1,6-diylbis(1h-imidazole); 1,2,4-triazole; 1,2,4-triazole-carboxamidine; 1,2-dichloro-propane; 1,2-dihydroxybenzene; 1,2-dimethoxyethane; 1,2-propanediol; 1,3,2-dioxaborolan-2-ol; 1,3,5-trichloro-benzene; 1,3-butanediol; 1,3-dihydroxyacetonephosphate; 1,3-diphenylurea; 1,3-propandiol; 1,3-thiazole-4-carboxylic acid; 1,4-butanediol; 1,4-diethylene dioxide; 1,4-dithio-alpha-d-mannose; 1,4-dithiothreitol; 1,6-diaminohexane; 1,6-dihydroxy naphthalene; 1,6-di-o-phosphono-d-allitol; 1,6-di-o-phosphono-d-mannitol; 1,6-fructose diphosphate (linear form); 1,8-di-hydroxy-4-nitro-anthraquinone; 1,8-di-hydroxy-4-nitro-xanthen-9-one; 1,n6-ethenoadenine; 10-cf3c(oh)2-ddacthf; 10-propargyl-5,8-dideazafolic acid; 11-deoxy-beta-rhodomycin; 11-mercaptoundecanoic acid; 12-bromododecanoic acid; 12-hydroxydodecanoic acid; 12-phenylheme; 1-3 sugar ring of pentamannosyl 6-phosphate; 13-acetylphorbol; 13beta-ethyl-17beta-hydroxygon-4-en-3-one; 16,17-androstene-3-ol; 16-bromoepiandrosterone; 19-nor-4-androstenediol; 19-nor-4-androstenedione; 19-nor-5-androstenediol; 19-nor-5-androstenedione; 19-norandrostenedione; 1-acetyl-2-carboxypiperidine; 1,2,5,8-tetrahydroxyanthracene-9,10-dione; 1,2,5-thiadiazolidin-3-one-1,1-dioxide; 1-amino-1-carbonyl pentane; 1-aminocyclopropanecarboxylic acid; 1-aminocyclopropylphosphonate; 1-androstenediol; 1-androstenedione; 1-anilino-8-naphthalene sulfonate; 1-benzyl-(r)-propylamine; 1-benzylimidazole; 1-beta-ribofuranosyl-1,3-diazepinone; 1-biphenyl-2-ylmethanamine; 1-bromopropane-2-ol; 1-butane boronic acid; 1-deaza-adenosine; 1′-deazo-thiamin diphosphate; 1-decane-sulfonic-acid; 1-deoxy-d-xylulose 5-phosphate; 1-deoxymannojirimycin; 1-deoxy-ribofuranose-5′-phosphate; 1-dodecanol; 1-ethyl-pyrrolidine-2,5-dione; 1-guanidinium-7-aminoheptane; 1h-benoximidazole-2-carboxylic acid; 1-hexadecanosulfonic acid; 1-hexadecanosulfonyl-o-l-serine; 1-hexadecylsulfonyl fluoride; 1h-indol-3-ylacetic acid; 1-hydroxy-1-thio-glycerol; 1-hydroxy-3-methylbutane; 1-methyl-2-quinolone; 1-methylcytosine; 1-methylimidazole; 1-methyloxy-4-sulfone-benzene; 1-monooleoyl-rac-glycerol; 1-octen-3-ol; 1-phenylcyclohexylamine; 1-proponol; 1-thio-beta-d-glucopyranose; 1-ethoxy-2-(2-ethoxyethoxy)ethane; 1-ethoxy-2-(2-methoxyethoxy)ethane; 1-deoxy-1-thio-heptaethylene glycol; 1-hydroxy-2-amino-3-cyclohexylpropane; 1-hydroxyamine-2-isobutylmalonic acid; 1-menaphthyl glutathione conjugate; 1-methoxy-2-(2-methoxyethoxy)ethane; 1-methoxy-2-[2-(2-methoxy-ethoxy]-ethane; 1-n-acetyl-beta-d-glucosamine; 1,2-diacyl-sn-glycero-3-phosphoinositol; 1,2-dipalmitoyl-phosphatidyl-glycerole; 1-piperidinocyclohexanecarbonitrile; 2-(1,1′-biphenyl-4-yl)propanoic acid; 2-(3,5-dimethylphenyl)-1,3-benzoxazole; 2-(3,6-dihydroxyphenyl)acetic acid; 2-(oxalyl-amino)-benzoic acid; 2-(phosphonooxy)butanoic acid; 2-(pyrido[1,2-e]purin-4-yl)amino-ethanol; 2(s)-amino-6-boronohexanoic acid; 2-(thiomethylene)-4-methylpentanoic acid; 2-(trimethylammonium)ethyl thiol; 2,2′:6′,2″-terpyridine platinum(ii); 2,3-anhydro-quinic acid; 2,3,4,5,6-pentafluorobenzyl alcohol; 2,3,5,6-tetrafluoro-4-methoxy-benzamide; 2′,3′-dideoxyadenosine-5′-triphosphate; 2′,3′-dideoxycytidine-5′-monophosphate; 2′,3-dideoxythymidine-5′-monophosphate; 2,3-dihydroxy-5-oxo-hexanedioate; 2,3-dimethyl-1,4-naphthoquinone; 2,3-di-o-sulfo-alpha-d-glucopyranose; 2,3-diphenyl-1h-indole-7-carboxylic acid; 2,4-diamino-4,6-dihydroxypyrimidine; 2,4-dihydroxy-3,3-dimethyl-butyrate; 2,4-dihydroxy-trans cinnamic acid; 2,5-dideoxy-2,5-imino-d-glucitol; 2,5-dimethoxy-4-ethylamphetamine; 2,5-dimethylpyrimidin-4-amine; 2,6-diamino-hexanoic acid amide; 2,6-diaminoquinazolin-4(3h)-one; 2-((4′-hydroxynaphthyl)-azo)benzoic acid; 2((4′-hydroxyphenyl)-azo)benzoic acid; 2-(1,3-thiazol-4-yl)-1h-benzimidazole-5-sulfonamide; 2-(1h-pyrrol-1-ylcarbonyl)benzene-1,3,5-triol; 2-(2-hydroxy-cyclopentyl)-pent-4-enal; 2-(1h-indol-3-yl)acetamide; (2r)-2-ethyl-1-hexanesulfonic acid; (2r)-2-phenyl-n-pyridin-4-ylbutanamide; 2-(1h-indol-3-yl)acetamide; 2-(1h-indol-3-yl)ethanamine; 2-(1h-indol-3-yl)ethanamine; 2-(1h-pyrazol-3-yl)-1h-benzimidazole; 2-(1h-pyrazol-3-yl)-1h-benzimidazole; 2-(3,4-dihydroxyphenyl)acetic acid; 2-(3-nitrophenyl)acetic acid; 2-(4-hydroxy-phenyl)benzofuran-5-ol; 2-(4-methoxyphenyl)acetamide; 2-(4-nitrophenyl)acetic acid; 2-(cyclohexylamino)benzoic acid; 2-(sec-butyl)thiazole; 2,3,-dihydroxybenzoylserine; 2,3-didehydroalanine; 2,3-dideoxyfucose; 2,3-difluorobenzyl alcohol; 2,3-dihydroxy-benzoic acid; 2,3-dihydroxy-valerianic acid; 2,3-dimethylimidazoli urn ion; 2′,4,4′-trihydroxychalcone; 2,4,6-triaminoquinazoline; 2,4,6-tribromophenol; 2,4,6-trinitrophenol; 2,4-diaminobutyric acid; 2,4-dihydroxybenzoic acid; 2,4-dinitrophenol; 2,5,7-trihydroxynaphthoquinone; 2,5-anhydroglucitol-1,6-biphosphate; 2,5-dimethoxyamphetamine; 2,5-xylidine; 2,6-diaminopimelic acid; 2,6-dibromo-4-phenoxyphenol; 2,6-dicarboxynaphthalene; 2′,6′-dichloro-biphenyl-2,6-diol; 2,6-difluorobenzenesulfonamide; 2,6-dimethyl-7-octen-2-ol; 2-[(dioxidophosphino)oxy]benzoate; 2′-5′dideoxyuridine; 2-acetyl-protoporphyrin ix; 2-allyl-6-methyl-phenol; 2-allylphenol; 2-amino-3-ketobutyric acid; 2-amino-4h-1,3-benzoxathiin-4-ol; 2-amino-5-hydroxy-benzimidazole; 2-amino-6-chloropyrazine; 2-amino-6-oxo-hexanoic acid; 2-amino-adenosine; 2-aminobenzoic acid; 2-aminobenzyl alcohol; 2-aminoethanesulfonic acid; 2-aminoethanimidic acid; 2-aminoethyl naphthalen-1-ylacetate; 2-amino-p-cresol; 2-amino-pentanoic acid; 2-aminophenol; 2-aminopimelic acid; 2-aminoprop-2-enamide; 2-aminopropanedioic acid; 2-aminoquinazolin-4(3h)-one; 2-aminothiazoline; 2-amino-vinyl-phosphate; 2-benzyl-3-iodopropanoic acid; 2-bromo-2-propene-1-ol; 2-bromo-6-chloro-purine; 2-bromo-6-hydroxy-purine; 2-bromoacetyl group; 2-bromophenol; 2-butanol; 2-chloro-5-nitro-n-phenylbenzamide; 2-chloro-6-methyl-aniline; 2′-chloro-biphenyl-2,3-diol; 2-chlorodideoxyadenosine; 2-chlorophenol; 2′-cytidylic acid; 2-dehydropantoate; 2-deoxy-2-aminogalactose; 2-deoxy-2-fluoro-i²-d-glucose; 2-deoxy-2-fluoro-i²-d-mannose; 2-deoxy-2-fluoro-i²-d-galactose; 2-deoxy-2-fluoro-i²-d-mannose; 2′-deoxyadenosine 5′-triphosphate; 2-deoxy-beta-d-galactose; 2′-deoxycytidine; 2-deoxyglucose; 2′-deoxyinosine; 2′-deoxymaltose; 2′-deoxyuridine; 2′-deoxyuridylic acid; 2-ethoxyethanol; 2-fluoro-2′-deoxyadenosine; 2-fluoroadenosine; 2-fluoroaniline; 2-formyl-protoporphryn ix; 2h-1-benzopyran-2-one; 2-hydroxyethyl disulfide; 2-hydroxy-tryptophan; 2-iminiopropanoate; 2-iminobiotin; 2-iodobenzylthio group; 2-isobutyl-3-methoxypyrazine; 2-keto-3-deoxygluconate; 2-methoxy-3-isopropylpyrazine; 2-methoxy-4-vinyl-phenol; 2-methoxyestradiol; 2-methoxyethanol; 2-methyl-2-propanol; 2-methylbutanoic acid; 2-methylleucine; 2-(n-morpholino)-ethanesulfonic acid; 2-methylpentane-1,2,4-triol; 2-naphthalenesulfonic acid; 2-nitro-p-cresol; 2′-o-butyl-5-methyluridine; 2-o-methyl fucose; 2-oxalosuccinic acid; 2-oxo-3-pentenoic acid; 2-oxo-4-methylpentanoic acid; 2-oxobutanoic acid; 2-oxoheptylphosphonic acid; 2-oxoquinoline; 2-phenyl-4h-benzo[h]chromen-4-one; 2-phenyl-4h-chromen-4-one; 2-phenylamino-ethanesulfonic acid; 2-phenyl-ethanol; 2-phenylethylamine; 2-phenylheme; 2-phospho-d-glyceric acid; 2-phosphoglyceric acid; 2-phosphoglycolic acid; 2-prolyl-5-tert-butyl-[1,3,4]oxadiazole; 2-propenyl-n-acetyl-neuramic acid; 2-propyl-aniline; 2-pyridinethiol; 2s,3s-3-methylaspartic acid; 2s,4r-4-methylglutamate; 2-tert-butylbenzene-1,4-diol; 2-thioethenamine; 2-thiomethyl-3-phenylpropanoic acid; 2-tridecanoyloxy-pentadecanoic acid; 2-amino-3-mercapto-propionamide; 2-amino-3-oxo-4-sulfo-butyric acid; 2-amino-4-butyl-5-propylselenazole; 2-amino-4-mercapto-butyric acid; 2-amino-n,3,3-trimethylbutanamide; 2-anhydro-3-fluoro-quinic acid; 2-carboxyethylphosphonic acid; 2-carboxypropyl-coenzyme a; 2-cyclopropylmethylenepropanal; 2-decenoyl n-acetyl cysteamine; 2′-deoxycytidine-5′-monophosphate; 2′-deoxycytidine-5′-triphosphate; 2-deoxy-glucitol-6-phosphate; 2-deoxy-glucose-6-phosphate; 2′-deoxyguanosine-5′-diphosphate; 2′-deoxyguanosine-5′-monophosphate; 2′-deoxyguanosine-5′-triphosphate; 2′-deoxy-thymidine-beta-l-rhamnose; 2′-deoxyuridine 3-monophosphate; 2-hydroxy-1,4-naphthoquinone; 2-hydroxy-3,5-diiodobenzoic acid; 3-chloro-4-hydroxyphenylglycine; 3-cyclohexyl-1-propylsulfonic acid; 3-(1,10-phenanthrol-2-yl)-l-alanine; 3-deazo-thiamin diphosphate; 3-(1-aminoethyl)nonanedioic acid; 3-(3,4-dimethoxyphenyl)propionic acid; 3-(4-hydroxy-phenyl)pyruvic acid; 3-(4-nitro-phenoxy)-propan-1-ol; 3-(4-nitrophenyl)-1h-pyrazole; 3-(benzoylamino)-l-alanine; 3-(benzyloxy)pyridin-2-amine; 3-(heptyloxy)benzoic acid; 3-(indol-3-yl) lactate; 3-(mercaptomethylene)pyridine; 3-(p-to1yl)propionic acid; 3,3′,5,5′-tetraiodothyroacetic acid; 3,4,5-trimethoxyamphetamine; 3,4-dichloroisocoumarin; 3,4-dihydrouracil; 3,4-dihydroxybenzoic acid; 3,4-dihydroxycinnamic acid; 3,4-dimethylaniline; 3,4-dimethylphenol; 3,5 dibromotyrosine; 3,5-diaminophthalhydrazide; 3,5-dibromobiphenyl-4-ol; 3,5-difluoroaniline; 3,5-diiodotyrosine; 3,5-dinitrocatechol; 3,6,9,12,15-pentaoxaheptadecane; 3,9-dimethyladenine; 3-acetyl pyridine adenine dinucleotide; 3-acetylpyridine adenine dinucleotide; 3-allylfentanyl; 3-amino-1-chloro-4-phenyl-butanol-2-yl; 3-amino-3-oxopropanoic acid; 3-amino-4-oxybenzyl-2-butanone; 3-amino-5-phenylpentane; 3-amino-6-hydroxy-tyrosine; 3-amino-alanine; 3-aminosuccinimide; 3-bromo-3-buten-1-ol; 3-bromo-7-nitroindazole; 3-butylthiolane 1-oxide; 3-chloroalaninate; 3-chlorophenol; 3-deaza-adenosine; 3-deazacytidine; 3-dehydroquinic acid; 3-dehydroshikimate; 3-deoxy-d-glucosamine; 3-deoxyguanosine; 3-fluoro-2-methyl-aniline; 3-fluorosialic acid; 3-fluorotyrosine; 3h-pyrazolo[4,3-d]pyrimidin-7-ol; 3-hydroxy-3-methyl-glutaric acid; 3-hydroxyanthranilic acid; 3-hydroxybutyryl-coenzyme a; 3-hydroxyglutaric acid; 3-hydroxyhippuric acid; 3-hydroxyimino quinic acid; 3-hydroxyisoxazole-4-carboxylic acid; 3-hydroxy-myristic acid; 3-hydroxyphenylalanine; 3-hydroxy-propanoic acid; 3-hydroxypyruvic acid; 3-indolebutyric acid; 3-iodo-benzyl alcohol; 3-iodo-tyrosine; 3-isopropylmalic acid; 3-methoxybenzamide; 3-methyladenine; 3-methyl-aspartic acid; 3-methyl-benzene-1,2-diol; 3-methylcytosine; 3-methylfentanyl; 3-methylglutamic acid; 3-methylphenylalanine; 3-methylpyridine; 3-methylthiofentanyl; 3-nitrophenylboronic acid; 3-oxiran-2ylalanine; 3′-oxo-adenosine; 3-oxo-pentadecanoic acid; 3-phenyl-1,2-propandiol; 3-phenylpropionic acid; 3-phenylpropylamine; 3-phenylpyruvic acid; 3-phosphoglyceric acid; 3-phosphoglycerol; 3-pyridin-4-yl-1h-indazole; 3r-hydroxydecanoyl-coa; 3-sulfinoalanine; 3-sulfooxy-1h-indole; 3-thiaoctanoyl-coenzyme a; 3′-thio-thymidine-5′-phosphate; 3-trimethylsilylsuccinic acid; 3′-uridinemonophosphate; 3-o-acetylthymidine-5′-diphosphate; 3-o-methylfructose in linear form; 3-phenyl-5-(1h-pyrazol-3-yl)isoxazole; 4-(4-fluorobenzyl)piperidine; 4-(dimethylamino)benzoic acid; 4-(1h-imidazol-1-yl)phenol; 4-(1-methyl-1-phenylethyl)phenol; 4-(2-amino-1,3-thiazol-4-yl)phenol; 4-(2-aminoethyl)-2-cyclohexylphenol; 4-(2-aminoethyl)-2-ethylphenol; 4-(2-thienyl)butyric acid; 4-(4-chlorophenyl)imidazole; 4-(carboxyvin-2-yl)phenylboronic acid; 4-(hydroxymercury)benzoic acid; 4-(hydroxymethyl)benzamidine; 4,4′-biphenyldiboronic acid; 4,4′-cyclohexane-1,1-diyldiphenol; 4,4′-dipyridyl disulfide; 4,4′-propane-2,2-diyldiphenol; 4,5-dehydro-d-glucuronic acid; 4,5-dehydro-l-iduronic acid; 4,6-dideoxy-4-amino-alpha-d-glucose; 4,6-dideoxy-4-amino-beta-d-glucopyranoside; 4,6-dideoxyglucose; 4,7-dimethyl-[1,10]phenanthroline; 4,7-dioxosebacic acid; 4-acetamidobenzoic acid; 4-amido-4-carbamoyl-butyric acid; 4-amino hexanoic acid; 4-aminobenzoic acid; 4-aminohydrocinnamic acid; 4-androstenediol; 4-androstenedione; 4-bromo-2,5-dimethoxyamphetamine; 4-bromo-2,5-dimethoxyphenethylamine; 4-carboxy-4-aminobutanal; 4-carboxycinnamic acid; 4-carboxyphenylboronic acid; 4-chlorobenzoic acid; 4-deoxy-4-amino-beta-d-glucose; 4-deoxy-alpha-d-glucose; 4-deoxy-d-glucuronic acid; 4-deoxy-d-mannuronic acid; 4-deoxyglucarate; 4-deoxylactose; 4-flourobenzenesulfonamide; 4-fluorobenzylamine; 4-fluorofentanyl; 4-fluorophenethyl alcohol; 4-fluorotryptophane; 4-guanidinobenzoic acid; 4-hydroperoxy-2-methoxy-phenol; 4-hydroxy-19-nortestosterone; 4-hydroxy-2-butanone; 4-hydroxy-3-methoxybenzoate; 4-hydroxy-aconitate ion; 4-hydroxybenzoyl coenzyme a; 4-hydroxybenzyl coenzyme a; 4-hydroxybutan-1-aminium; 4-hydroxycoumarin; 4′-hydroxyflavanone; 4-hydroxyphenacyl coenzyme a; 4-hydroxyphenylglycine; 4-hydroxytestosterone; 4-iodo-d-phenylalanine; 4-iodo-1-phenylalanine; 4-iodophenol; 4-iodopyrazole; 4-methoxyamphetamine; 4-methoxy-e-rhodomycin t; 4-methyl valeric acid; 4-methyl-1,2-benzenediol; 4-methyl-2,5-dimethoxyamphetamine; 4-methyl-2h-chromen-2-one; 4-methylaminorex; 4-methyl-histidine; 4-methylidene-5-one; 4-methylimidazole; 4-methylpiperazin-1-ylcarbonyl group; 4-methyl-pyrroline-5-carboxylic acid; 4-methylthio-alpha-d-mannose; 4-methylumbelliferyl chitobiose; 4-methylumbelliferyl-alpha-d-glucose; 4-nitrocatechol; 4-nitro-inden-1-one; 4-nitrophenyl phosphate; 4′-nitrophenyl-3i-thiolaminaritrioside; 4-nitrophenyl-ara; 4-nitrophenyl-beta-d-galactoside; 4-o-methyl-alpha-d-glucuronic acid; 4-o-methyl-beta-d-glucuronic acid; 4-oxoretinol; 4-oxosebacic acid; 4-phenyl-1h-imidazole; 4-phenylbutylamine; 4-phenylfentanyl; 4-phospho-d-erythronate; 4-phospho-d-erythronohydroxamic acid; 4′-phosphopantetheine; 4-piperidin-4-ylbutanal; 4-piperidino-piperidine; 4r-fluoro-n6-ethanimidoyl-1-lysine; 4-(1,3-benzoxazol-2-yl)-2,6-dibromophenol; 4-(1,3-benzoxazol-2-yl)-2,6-dimethylphenol; 4-(2,2,2-trifluoroethyl)-1-phenylalanine; 4-thio-beta-d-glucopyranose; 5-(2-chlorobenzyl)-2-furoic acid; 5-(2-chlorophenyl)-1,3,4-thiadiazole-2-sulfonamide; 5-(2-chlorophenyl)furan-2-carbohydrazide; 5-(2-chlorophenyl)furan-2-carboxylic acid; 5-(2-hydroxyethyl)nonane-1,9-diol; 5-(2-methoxyphenyl)-2-furoic acid; 5-(2-nitrophenyl)-2-furoic acid; 5,10-dideazatetrahydrofolic acid; 5,10-methylene-6-hydrofolic acid; 5,4′-dideoxyflavanone; 5,6-cyclic-tetrahydropteridine; 5,6-diaminouracil; 5,6-dihydroxy-nadp; 5,6-dimethylbenzimidazole; 5,8-di-amino-1,4-dihydroxy-anthraquinone; 5,8-dimethoxy-1,4-dimethylquinolin-2(1h)one; 5-[2-(1h-pyrrol-1-yl)ethoxy]-1h-indole; 5alpha-androstane-3alpha,17beta-diol; 5-alpha-androstane-3-beta,17-alpha-diol; 5-alpha-androstane-3-beta,17beta-diol; 5-amidino-benzimidazole; 5-amino 6-nitro uracil; 5-amino-1h-pyrimidine-2,4-dione; 5-aminoimidazole ribonucleoside; 5-aminoisoquinoline; 5-aminonaphthalene-1-sulfonic acid; 5-androstenedione; 5-benzyl-1,3-thiazol-2-amine; 5-beta-androstane-3,17-done; 5-beta-dihydrotestosterone; 5-bromo-cytidinemonophosphate; 5-bromonicotinamide; 5-bromothienyldeoxyuridine; 5-bromovinyldeoxyuridine; 5-chloryl-2,4,6-quinazolinetriamine; 5′-deoxy-5′-(methylthio)-tubercidin; 5′-deoxy-5′-methylthioadenosine; 5′-deoxy-5′-piperidin-1-ylthymidine; 5-deoxyflavanone; 5-exo-hydroxycamphor; 5′-fluoro-2′,5′-dideoxyadenosine; 5-fluoro-2′-deoxyuridine-5′-monophosphate; 5-fluoro-4-(s)-hydroxy-3,4-dihydropyrimidine; 5′-fluoro-5′-deoxyadenosine; 5-fluoro-beta-l-gulosyl fluoride; 5-fluoroindole propanol phosphate; 5-fluorolevulinic acid; 5-fluorouridine; 5-formyl-6-hydrofolic acid; 5-hydroxy norvaline; 5-hydroxymethyl-chonduritol; 5-hydroxyvaleric acid; 5-iodotubercidin; 5-iodouracil; 5-methoxybenzimidazole; 5-methylbenzimidazole; 5-methylcytidine-5′-monophosphate; 5-methylpyrrole; 5-methyltetrahydrofolate; 5-methyluridine 5-monophosphate; 5-n-allyl-arginine; 5-nitroindazole; 5-oxo-1-norleucine; 5-oxo-pyrrolidine-2-carbaldehyde; 5-pentyl-2-phenoxyphenol; 5-phenyl-1h-indazol-3-amine; 5-phenyl-2-keto-valeric acid; 5-phenylsulfanyl-2,4-quinazolinediamine; 5-phenylvaleric acid; 5-phosphoarabinonic acid; 5′-s-ethyl-5-thioadenosine; 6-(dihydroxy-isobutyl)-thymine; 6-amino-1-methylpurine; 6-aminobenzoic acid; 6-aminohexyl-uridine-c1,5′-diphosphate; 6-aza-ump; 6-bromo-1-hexanol; 6-carboxymethyluracil; 6-chloro-2-fluoropurine; 6-deoxy-alpha-d-glucose; 6-deoxyerythronolide b; 6-deoxyglucose; 6-hydroxy-d-norleucine; 6-hydroxydopa quinone; 6-hydroxy-1-norleucine; 6-hydroxymethyl-7,8-dihydropterin; 6-hydroxymethylpterin; 6-hydroxypropylthymine; 6-hydroxyuridine-5′-phosphate; 6-methylamino-5-nitroisocytosine; 6-methyl-formycin a; 6-methylpurine; 6′-methyl-thiamin diphosphate; 6-morpholin-4-yl-9h-purine; 6-nitroindazole; 6-o-cyclohexylmethyl guanine; 6-phosphogluconic acid; 6s-5,6,7,8-tetrahydrobiopterin; 7,8-diamino-nonanoic acid; 7,8-dihydrobiopterin; 7,8-dihydroneopterin; 7,8-dimethylalloxazine; 7,9-dimethylguanine; 7a-methyl-19-nortestosterone; 7-deaza-7-aminomethyl-guanine; 7-deaza-7-cyano-guanine; 7-deazaguanine; 7-hydroxystaurosporine; 7-iodo-1,2,3,4-tetrahydro-isoquinoline; 7-keto-8-aminopelargonic acid; 7-methyl-gpppa; 7-methylguanosine; 7-methyl-guanosine-5′-triphosphate; 7-nitroindazole; 7-nitroindazole-2-carboxamidine; 8-azaguanine; 8-azaxanthine; 8-bromoadenosine-5′-diphosphate; 8-hydroxy-2′-deoxyguanosine; 8-iodo-guanine; 8-methyl-9-oxoguanine; 99mtc-ciprofloxacin; 9-aminophenanthrene; 9-deazaadenine; 9-deazaguanine; 9-deazahypoxanthine; 9-deazainosine; 9h-carbazole; 9-hydroxypropyladenine, r-isomer; 9-hydroxypropyladenine, s-isomer; 9-methyl uric acid; 9-methylguanine; 9-n-phenylmethylamino-tacrine; 9r,13r-opda; 17alpha-methyl-4-hydroxynandrolone; and/or 17alpha-methyl-delta1-dihydrotestosterone.

In additional embodiments, at least one of the following compounds may be used as perturbation stimuli: 17-MG (Tanespimycin)/KOS-953, 22S-Hydroxycholesterol, 740 Y-P, A3-HCl, ABT-263, ACEA, Acetaminophen (paracetamol), Acetycholine, Activin, Adapin (doxepin), Afatinib, AICAR, Alvespimycin hydrochloride, AM 580, AMG 337, Amigal (Deoxygalactonojirimycin hydrochloride), Amiodarone, Amlexanox, Amlodipine Besylate, Amuvatinib, Anti mullerian hormone, APS-2-79, ARN-509, AST-1306, atenolol, ATRA, Axitinib, AZD1080, AZD1208, AZD1480, AZD2858, AZD6738, AZD-8055, AZD8931, bafetinib, BAM7, Bardoxolone, baricitinib, BAY 87-2243, BDA-366, Benzbromarone, b-Estradiol, Bethanechol chloride, Bexarotene, BGJ398, BIIB021, BIO, BIRB 796, Bitopertin, BMP2, BMP2, BMP4, BMS 195614, bms189453, BMS-754807, BMS777607, bms833923, bms-986094 (inx-189), Bosutinib, Bryostatint BSI-201, BX795, Cabozantinib (XL184, BMS-907351), Calcitriol, CALP1, CALP3, Captopril, CCCP, CD 2665, Cediranib, cerdulatinib, Ceritinib, CGK733, CH223191, Chloroquine phosphate, CI-4AS-1, Clozapine, CMX001 (Brincidofovir), CO-1686 (Rociletinib), Cobalt chloride, Compound Name, Corticosterone, CP-673451, CP690550 citrate, Crenolanib, Crizotinib, Cryptotanshinone, CZ415, Dacomitinib (PF299804, PF299), Dalcetrapib, Danazol, Darapladib, Dasatinib, Decernotinib, decernotinib, decernotinib, Deferoxamine mesylate, Dexamethasone, Disopyramide, DLPC, dmPGE2, DMXAA, Dopamine, dorsomorphin, Dorsomorphin, DPH, Duvelisib, DY 268, EB1089, Echinomycin, EGF, Eletriptan, entospletinib, Enzastaurin, enzastaurin, Epinephrine, Erlotinib, Etomidate, Evacetrapib, everolimus, EVP-6124 (hydrochloride) (encenicline), EW-7197, Ezutromid, Fexofenadine, FGF, FICZ, Fluoxymesterone, Forskolin, FRAX597, Ganaxolone, GDC-0879, GDC-0941, GDC-0994, GDF10 (BMP3b), GDF2 (BMP9), Glasdegib (PF-04449913), Glimepiride, GLPG0634 analog, GLPG0634/Filgotinib, Glucose, Glycopyrrolate, G06983, GSK1904529A, GSK2126458, GSK2334470, GSK621, GW 7647, GW3965, GW4064, GZD824 Dimesylate, HGF/SF, Histamine, HS-173, HSP-990, Huperzine, Hydrocortisone, ibrutinib, Ibuprofen, idelalisib, IFN-y, IGF-1, imatinib, inhibin A, INK-128, INNO-206 (aldoxorubicin), Insulin, Ipatasertib, IPI-493, Irinotecan Hydrochloride Trihydrate, isoniazid, itacitinib, IWP-2, JANEX-1, JNJ-38877605, K02288, Kartogenin, KU-60019, Laropiprant (tredaptive), Latrepirdine, Latrepirdine (dihydrochloride), LDN193189, LDN193189, LDN-212854, Leucine, LJI308, LRH-1 antagonist, LY 2140023 (Pomaglumetad methionil-LY404039), LY 364947, LY2584702, LY2784544, LY294002, LY303511 HCl, LY404039 (pomaglumetad methionil (mGlu2/3)), LYS2784544, Masitinib, meB10, Merestinib, Mefformin, Methapyrilene, MHY1485, M1-773, Mifepristone, MK-0752, Momelotinib, MRT67307, N-Acetylpurinomycin, nandrolone decanoate, Nefazodone, Netarsudil, nilotinib, nilvadipine, Nitrofurantoin, Nodal, NOV-002 (oxidized L-Glutathione), NSC228155, NVP-AUY922, oclacitinib maleate, Oligomycin A, Omeprazole, 0S1-027, OSU-03012, Otenabant, Oxoglaucine, Pacritinib (SB1518), PAF C-16, Palifosfamide, Papaverine, PCI-32765, PDGF, Penicillamine(D-), Peramivir (trihydrate), Perifosine (KRX-0401), Perphenazine, PF-04691502, PF-04691502, PF-04929113 (SNX-5422), PHA-665752, Phenformin, Phorbo112,13-dibutyrate, Picropodophyllin, Pifithrin-a, Pifithrin-u, PKI-179, PND-1186, Pomalidomide, ponatinib, PP242, prednisone, Preladenant, PRI-724, Propylthiouracil, Purmorphamine, Quizartinib, R406, R788 (fostamatinib, disodium hexahydrate), Ranitidine, rapamycin, Regorafenib, Retaspimycin Hydrochloride, Rifampicin, Rimonabant, RITA (NSC 652287), Ritonavir, R04929097, Rolofylline (KW-3902), Rosiglitazone maleate, RU 28318, ruxolitinib/jakafi, SC75741, SC79, Selumetinib, Semagacestat, Semaxinib, SG1-1776, SH-4-54, Simvastatin, SIS3, SKL2001, SMI-4a, Smoothened Agonist, solcitinib/GSK2586184, Sotrastaurin, SP600125, STA-21, Sulpiride, T0070907, T0901317, TAK-593, TAK-659 HCl, Taladegib, Taltorvic/MK-8669, Deforolimus, Ridaforolimus, Tamibarotene, Tamsulosin hydrochloride, Tandutinib, TC-5214 (R-Mecamylamine hydrochloride), TC-5214 (S-(+)-MecaMylaMine Hydrochloride), TCS 359, Temsirolimus, TFP, TG-101348, TGF-b, TGX-221, TH-302 (Evofosfamide), Thalidomide, Tiagabine, Tipifarnib, Tivozanib, TNF-α, Tofacitinib citrate, Torcetrapib, torin1, TP-434 (Eravacycline), Transferrin, Trifluoperazine, upadacitinib, Valproate, Vanoxerine (dihydrochloride), Varenicline Tartrate, Vicriviroc (maleate), Voxtalisib, XL765, SAR245409, VX-661 (tezacaptor), WAY600, Wnt agonist 1, Wnt3a, WYE-125132, WYE-125132 (WYE-132), Xav939, XL228, XMU-MP-1, Zibotentan, or a-PHP.

### Downstream Targets

In certain embodiments, a stimulus is administered that targets a downstream product of a gene of a GSN. Alternatively, the stimulus disrupts a GSN that affects downstream expression of at least one downstream target. mRNA

Perturbation of single or multiple GSNs associated with a single insulated neighborhood or across multiple INs can affect the transcription of a single gene or a multiple set of genes by altering the boundaries of the IN due to loss of anchor sites comprising cohesins. Specifically, perturbation of a GSC may also affect the transcription of a single gene or a multiple set of genes. Perturbation stimuli may result in the modification of the RNA expression and/or the sequences in the primary transcript within the mRNA, i.e., the exons or the RNA sequences between the exons that are removed by splicing, i.e. the introns. Such changes may consequently after the members of the set of signaling molecules/signaling proteins within a GSC of the GSN of a gene, thereby defining a variant of the GSN.

### Proteins

Perturbation of single or multiple GSNs associated with a single insulated neighborhood or across multiple insulated neighborhoods can affect the translation of a single gene or a multiple set of genes that are part of the single or multiple GSNs, as well as those downstream. Specifically, perturbation of a GSC may affect translation. Perturbation may result in the inhibition of the translated protein. In certain embodiments, the binding profile of a GSC is perturbed to affect translation of a protein.

### Nearest Neighbor Gene

Perturbation stimuli may cause interactions with signaling molecules/signaling proteins to occur in order to after expression of the nearest primary neighborhood gene that may be located upstream or downstream of the primary neighborhood gene. Neighborhood genes may have any number of upstream or downstream genes along the chromosome. Within any IN, there may be one or more, e.g., one, two, three, four or more, upstream and/or downstream neighborhood genes relative to the primary neighborhood gene. A “primary neighborhood gene” is a gene which is most commonly found within a specific IN along a chromosome. An upstream neighborhood gene of a primary neighborhood gene may be located within the same IN as the primary neighborhood gene. A downstream neighborhood gene of a primary neighborhood gene may be associated the same IN or be regulated by the same GSC as the primary neighborhood gene.

### Canonical Cell Signaling Pathways

It is understood that there may be some overlap between the canonical pathways detailed in the art and the GSNs defined herein.

Whereas canonical pathways permit a certain degree of promiscuity of members across pathways (cross talk), GSNs of the invention are defined at the gene level and characterized based on any number of stimuli or perturbation to the cell, tissue, organ or organ system expressing that gene. Hence the nature of a GSN is both structurally (e.g., the gene) and situationally (e.g., the function, e.g., expression profile) defined. And while two different gene signaling networks may share members, they are still unique in that the nature of the perturbation can distinguish them. Hence, the value of GSNs in the elucidation of the function of biological systems in support of therapeutic research and development.

It should be understood that it is not intended that no connection ever be made between canonical pathways and GSNs; in fact, the opposite is the case. In order to bridge the two signaling paradigms for further scientific insights, it will be instructive to compare the canonical signaling pathway paradigm with the GSNs of the present invention.

Canonical pathways which may be altered according to the present invention include, but are not limited to the 2-arachidonoylglycerol biosynthesis pathway, 2-oxocarboxylic acid metabolism pathway, 5HT1 type receptor mediated signaling pathway, 5HT2 type receptor mediated signaling pathway, 5HT3 type receptor mediated signaling pathway, 5HT4 type receptor mediated signaling pathway, 5-hydroxytryptamine biosynthesis pathway, 5-hydroxytryptamine degradation pathway, abacavir transport and metabolism pathway, ABC transporters pathway, ABC-family proteins mediated transport pathway, ACE inhibitor pathway, acetate utilization pathway, acetylcholine synthesis pathway, activation of camp-dependent PKA pathway, activin beta signaling pathway, adenine and hypoxanthine salvage pathway, adherens junction pathway, adipocytokine signaling pathway, adipogenesis pathway, adrenaline and noradrenaline biosynthesis pathway, adrenergic signaling in cardiomyocytes pathway, advanced glycation end-products (age/rage) pathway, advanced glycosylation end product receptor signaling pathway, aflatoxin b1 metabolism pathway, age/rage pathway, AHR pathway, AKT signaling pathway, alanine and aspartate metabolism pathway, alanine biosynthesis pathway, aldosterone synthesis and secretion pathway, aldosterone-regulated sodium reabsorption pathway, allantoin degradation pathway, allograft rejection pathway, all-trans-retinoic acid signaling pathway, alp23b signaling pathway, alpha 6 beta 4 signaling pathway, alpha adrenergic receptor signaling pathway, alpha6 beta4 integrin pathway, alpha-linolenic acid metabolism pathway, Alzheimer disease-amyloid secretase pathway, Alzheimer disease-presenilin pathway, amino acid conjugation pathway, amino sugar and nucleotide sugar metabolism pathway, aminoacyl-tRNA biosynthesis pathway, aminobutyrate degradation pathway, AMP-activated protein kinase pathway, AMPK signaling pathway, anandamide biosynthesis pathway, anandamide degradation pathway, androgen receptor signaling pathway, androgen/estrogen/progesterone biosynthesis pathway, angiogenesis pathway, angiopoietin-Tie2 signaling pathway, angiotensin II-stimulated signaling through g proteins and beta-arrestin pathway, antigen processing and presentation by MHC's pathway, apoptosis modulation and signaling pathway, apoptosis modulation by HSP70 pathway, apoptosis signaling pathway, apoptosis through death receptors pathway, apoptotic execution phase pathway, arachidonate epoxygenase/epoxide hydrolase pathway, arachidonic acid metabolism pathway, arginine and proline metabolism pathway, arginine biosynthesis pathway, aripiprazole metabolic pathway, arylamine metabolism pathway, ascorbate and aldarate metabolism pathway, ascorbate degradation pathway, asparagine and aspartate biosynthesis pathway, asparagine N-linked glycosylation pathway, aspartate and glutamate metabolism pathway, assembly of RNA polymerase-II initiation complex pathway, ATM pathway, ATP synthesis pathway, axon guidance pathway, axon guidance mediated by netrin pathway, axon guidance mediated by semaphorins pathway, axon guidance mediated by slit/robo pathway, B cell activation pathway, B cell receptor (BCR) pathway, B cell receptor signaling pathway, bacterial invasion of epithelial cells pathway, basal transcription factors pathway, base excision repair pathway, B-cell development pathway, B-cell receptor pathway, B-cell receptor complex pathway, benzo pathway, betal adrenergic receptor signaling pathway, beta2 adrenergic receptor signaling pathway, beta3 adrenergic receptor signaling pathway, beta-alanine metabolism pathway, bile acid and bile salt metabolism pathway, bile secretion pathway, binding and uptake of ligands by scavenger receptors pathway, biogenic amine synthesis pathway, biosynthesis of amino acids pathway, biosynthesis of unsaturated fatty acids pathway, biotin biosynthesis pathway, blakely network pathway, blood clotting cascade pathway, blood coagulation pathway, bmp/activin signaling-Drosophila pathway, bone morphogenic protein pathway, brain-derived neurotrophic factor (BDNF) pathway, BRCA1 pathway, bupropion degradation pathway, butanoate metabolism pathway, butirosin and neomycin biosynthesis pathway, butyrate-induced histone acetylation pathway, cadherin signaling pathway, caffeine metabolism pathway, calcium regulation in the cardiac cell pathway, calcium signaling pathway, cAMP pathway, carbohydrate digestion and absorption pathway, carbon metabolism pathway, cardiac muscle contraction pathway, cardiac progenitor differentiation pathway, carnitine metabolism pathway, caspase cascade pathway, catalytic cycle of mammalian flavin-containing monooxygenases pathway, CCKR signaling map pathway, CCR5 in macrophages pathway, CD4 and CD8 T-cell lineage pathway, CD40 signaling pathway, CDK5 pathway, cell adhesion molecules (cams) pathway, cell cycle checkpoints pathway, cell cycle pathway, cell differentiation-meta pathway, cell junction organization pathway, cell surface interactions at the vascular wall pathway, CGMP-PKG signaling pathway, chemical carcinogenesis pathway, chemokine signaling pathway, cholesterol biosynthesis pathway, cholinergic synapse pathway, chorismate biosynthesis pathway, chromatin remodeling pathway, circadian clock system pathway, circadian entrainment pathway, citrate cycle (TCA cycle) pathway, c-met pathway, cobalamin biosynthesis pathway, codeine and morphine metabolism pathway, coenzyme A biosynthesis pathway, coenzyme A linked carnitine metabolism pathway, colchicine metabolic pathway, collecting duct acid secretion pathway, complement and coagulation cascades pathway, cori cycle pathway, corticotropin-releasing hormone (CRH) pathway, costimulation by the CD28 family pathway, CREB pathway, CTL mediated apoptosis pathway, CTLA4 signaling pathway, cyanoamino acid metabolism pathway, cyclins and cell cycle regulation pathway, cysteine and methionine metabolism pathway, cysteine biosynthesis pathway, cytokine network pathway, cytokine-cytokine receptor interaction pathway, cytokines and inflammatory response pathway, cytoplasmic ribosomal proteins pathway, cytoskeletal regulation by rho GTPase pathway, cytosolic DNA-sensing pathway, de novo purine biosynthesis pathway, de novo pyrimidine deoxyribonucleotide biosynthesis pathway, de novo pyrimidine ribonucleotides biosynthesis pathway, depolarization of the presynaptic terminal triggers the opening of calcium channels pathway, diclofenac metabolic pathway, differentiation pathway, digestion resistant carbohydrate metabolism pathway, dilated cardiomyopathy pathway, dissolution of fibrin clot pathway, diurnally regulated genes with circadian orthologs pathway, div no colors pathway, div pathway, DNA damage bypass pathway, DNA damage response pathway, DNA damage reversal pathway, DNA methylation and transcriptional repression pathway, DNA repair mechanisms pathway, DNA replication pathway, dopamine metabolism pathway, dopamine receptor mediated signaling pathway, dopaminergic synapse pathway, dorso-ventral axis formation pathway, DPP signaling pathway, DPP-SCW signaling pathway, drug metabolism pathway, drug metabolism-cytochrome p450 pathway, dscam interactions pathway, E2F/MIRHG1 feedback-loop-delete pathway, EBV LMP1 signaling pathway, ECM-receptor interaction pathway, effects of nitric oxide pathway, effects of pip2 hydrolysis pathway, EGF pathway, EGF receptor signaling pathway, eicosanoid synthesis pathway, electron transport chain pathway, endochondral ossification pathway, endocrine and other factor-regulated calcium reabsorption pathway, endocytosis pathway, endoderm differentiation pathway, endogenous cannabinoid signaling pathway, endothelin pathway, endothelin signaling pathway, energy metabolism pathway, enkephalin release pathway, enos signaling pathway, ephrin-EPHR signaling pathway, epidermal growth factor receptor (EGFR) pathway, epithelial cell signaling in Helicobacter pylori infection pathway, epithelial tight junctions pathway, EPO receptor signaling pathway, ERBB signaling pathway, ERK signaling pathway, erythropoietin pathway, estrogen signaling pathway, ether lipid metabolism pathway, eukaryotic transcription initiation pathway, eukaryotic translation elongation pathway, eukaryotic translation initiation pathway, eukaryotic translation termination pathway, FAK1 signaling pathway, Fas signaling pathway, fat digestion and absorption pathway, fatty acid pathway, fatty acid beta oxidation pathway, fatty acid biosynthesis pathway, fatty acid degradation pathway, fatty acid elongation pathway, fatty acid metabolism pathway, fatty acid omega oxidation pathway, FGF pathway, FGF signaling pathway, fibroblast growth factor-1 (FGF1) pathway, flavin biosynthesis pathway, FLT3 signaling pathway, fluoropyrimidine activity pathway, focal adhesion pathway, folate biosynthesis pathway, folate metabolism pathway, follicle stimulating hormone pathway, formation of fibrin clot pathway, formyltetrahydroformate biosynthesis pathway, Foxo signaling pathway, fructose galactose metabolism pathway, G protein signaling pathway, G1 to S cell cycle control pathway, G13 signaling pathway, GABA synthesis pathway, GABA-B receptor II signaling pathway, galactose metabolism pathway, gamma-aminobutyric acid synthesis pathway, ganglio sphingolipid metabolism pathway, gap junction trafficking and regulation pathway, gastric acid secretion pathway, gastrin pathway, GBB signaling pathway, generic transcription pathway, ghrelin pathway, glial cell differentiation pathway, globo sphingolipid metabolism pathway, glucagon signaling pathway, glucocorticoid & mineralcorticoid metabolism pathway, glucocorticoid receptor signaling pathway, glucose homeostasis pathway, glucuronidation pathway, glutamatergic synapse pathway, glutamine glutamate conversion pathway, glutathione metabolism pathway, glycan degradation pathway, glycerolipid metabolism pathway, glycerophospholipid biosynthetic pathway, glycerophospholipid metabolism pathway, glycine metabolism pathway, glycogen metabolism pathway, glycolysis/gluconeogenesis pathway, glycosaminoglycan biosynthesis-heparan sulfate/heparin pathway, glycosaminoglycan biosynthesis-keratan sulfate pathway, glycosaminoglycan degradation pathway, glycosaminoglycan metabolism pathway, glycosphingolipid biosynthesis—ganglio series pathway, glycosphingolipid biosynthesis-globo series pathway, glycosphingolipid biosynthesis—lacto and neolacto series pathway, glyoxylate and dicarboxylate metabolism pathway, gonadotropin-releasing hormone receptor pathway, GP1B-IX-V activation signaling pathway, GPCR pathway, GPCR downstream signaling pathway, GPCR ligand binding pathway, GPVI-mediated activation cascade pathway, granulocyte adhesion and diapedesis pathway, granzyme pathway, growth hormone signaling pathway, GSK 3 signaling pathway, hedgehog signaling pathway, hematopoiesis from pluripotent stem cells pathway, hematopoietic cell lineage pathway, hematopoietic stem cell differentiation pathway, heme biosynthesis pathway, hepatitis B pathway, hepatitis C pathway, heterotrimeric G-protein signaling-Gi alpha and Gs alpha mediated pathway, heterotrimeric g-protein signaling-rod outer segment phototransduction pathway, hexose transport pathway, HGF pathway, HIF-1 signaling pathway, hippo signaling pathway, histamine h1 receptor mediated signaling pathway, histamine h2 receptor mediated signaling pathway, histamine synthesis pathway, histidine biosynthesis pathway, histone modifications pathway, homologous recombination pathway, HTLV-I infection pathway, human complement system pathway, hypoxia response via hif activation pathway, ID signaling pathway, IGF1R signaling pathway, IL1 and megakaryocytes in obesity pathway, IL-1 signaling pathway, IL-10 pathway, IL17 signaling pathway, IL-2 signaling pathway, IL-22 pathway, IL-3 signaling pathway, IL-4 signaling pathway, IL-5 signaling pathway, IL-6 pathway, IL-7 signaling pathway, IL-9 signaling pathway, ILK signaling pathway, inflammation mediated by chemokine and cytokine signaling pathway, inflammatory mediator regulation of Trp channels pathway, inflammatory response pathway, influenza a virus infection pathway, inos signaling pathway, inositol phosphate metabolism pathway, insulin receptor pathway, insulin resistance pathway, insulin secretion pathway, insulin/IGF-protein kinase b signaling cascade pathway, insulin-like growth factor-2 mRNA binding proteins pathway, integrin alphaIIb beta3 signaling pathway, integrin cell signaling pathway, integrin cell surface interactions pathway, integrin-mediated cell adhesion pathway, interferon pathway, interferon alpha/beta signaling pathway, interferon type I signaling pathway, interferon-gamma signaling pathway, interleukin signaling pathway, interleukin-1 (IL-1) pathway, interleukin-1 processing pathway, interleukin-11 signaling pathway, interleukin-2 (IL-2) pathway, interleukin-3 pathway, interleukin-4 (IL-4) pathway, interleukin-5 (IL-5) pathway, interleukin-6 (IL-6) pathway, interleukin-7 (IL-7) pathway, interleukin-9 (i1-9) pathway, intracellular calcium signaling pathway, ionotropic glutamate receptor pathway, IP3 pathway, isoleucine biosynthesis pathway, JAK/STAT pathway, JNK pathway, kinesins pathway, KIT receptor pathway, LDL oxidation in atherogenesis pathway, leptin (lep) pathway, leptin signaling pathway, leucine biosynthesis pathway, leukocyte transendothelial migration pathway, linoleic acid metabolism pathway, lipid digestion pathway, lipoate_biosynthesis pathway, longevity regulating—mammal pathway, longevity regulating—multiple species pathway, long-term potentiation pathway, lysine biosynthesis pathway, lysine degradation pathway, lysosome pathway, mannose metabolism pathway, MAPK cascade pathway, MAPK targets/nuclear events mediated by MAP kinases pathway, matrix metalloproteinases pathway, melatonin metabolism and effects pathway, meta biotransformation pathway, metabolism of carbohydrates pathway, metabolism of nitric oxide pathway, metabolism of nucleotides pathway, metabolism of porphyrins pathway, metabolism of water-soluble vitamins and cofactors pathway, metabolism of xenobiotics by cytochrome p450 pathway, metabotropic glutamate receptor group I pathway, metabotropic glutamate receptor group II pathway, metabotropic glutamate receptor group III pathway, methionine biosynthesis pathway, methylation pathway, methylcitrate cycle pathway, methylmalonyl pathway, mineral absorption pathway, miRNA biogenesis pathway, mismatch repair pathway, mitochondrial apoptosis pathway, mitochondrial gene expression pathway, mitochondrial Ic-fatty acid beta-oxidation pathway, mitotic G1-G1/S phases pathway, mitotic G2-G2/M phases pathway, monoamine GPCRs pathway, monoamine transport pathway, mRNA capping pathway, mRNA editing pathway, mRNA processing pathway, mRNA splicing pathway, mRNA surveillance pathway, mTOR signaling pathway, muscarinic acetylcholine receptor 1 and 3 signaling pathway, muscarinic acetylcholine receptor 2 and 4 signaling pathway, myogenesis pathway, myometrial relaxation and contraction pathway, N-acetylglucosamine metabolism pathway, NAD biosynthesis H pathway, nanomaterial induced apoptosis pathway, nanoparticle triggered autophagic cell death pathway, nanoparticle triggered regulated necrosis pathway, natural killer cell mediated cytotoxicity pathway, ncam signaling for neurite out-growth pathway, nephrin interactions pathway, netrin-1 signaling pathway, neural crest differentiation pathway, neuroactive ligand-receptor interaction pathway, neurotransmitter clearance in the synaptic deft pathway, neurotransmitter release cycle pathway, neurotransmitter uptake and metabolism in glial cells pathway, neurotrophin signaling pathway, NFAT and cardiac hypertrophy pathway, NF-kappa b signaling pathway, NF-kappa b signaling pathway, NGF pathway, NGF signaling via TRKA from the plasma membrane pathway, N-glycan biosynthesis pathway, nicotinate and nicotinamide metabolism pathway, nicotine activity on chromaffin cells pathway, nicotine activity on dopaminergic neurons pathway, nicotine degradation pathway, nicotine metabolism pathway, nicotine pharmacodynamics pathway, nicotinic acetylcholine receptor signaling pathway, nifedipine activity pathway, nitrogen metabolism pathway, NLR proteins pathway, nod-like receptor signaling pathway, non-homologous end joining pathway, notch signaling pathway, Nrf2 pathway, nuclear receptors pathway, nucleosome assembly pathway, nucleotide excision repair pathway, nucleotide GPCRs pathway, nucleotide metabolism pathway, nucleotide-binding oligomerization domain pathway, o-antigen biosynthesis pathway, o-glycan biosynthesis pathway, olfactory transduction pathway, oncostatin m signaling pathway, one carbon metabolism pathway, opioid prodporphin pathway, opioid proenkephalin pathway, opioid proopiomelanocortin pathway, ornithine degradation pathway, osteoblast signaling pathway, osteoclast signaling pathway, osteopontin signaling pathway, ovarian steroidogenesis pathway, oxidation by cytochrome p450 pathway, oxidative phosphorylation pathway, oxidative stress pathway, oxytocin receptor mediated signaling pathway, oxytocin signaling pathway, p38 MAPK signaling pathway, p53 feedback loops 1 pathway, p53 feedback loops 2 pathway, p53 mediated apoptosis pathway, p53 signaling pathway, pak pathway, pancreatic secretion pathway, pantothenate biosynthesis pathway, parkin-ubiquitin proteasomal system pathway, passive transport by aquaporins pathway, PDGF signaling pathway, pentose and glucuronate interconversions pathway, pentose phosphate pathway, peptide GPCRs pathway, peptidoglycan biosynthesis pathway, peroxisomal beta-oxidation of tetracosanoyl-coA pathway, peroxisomal lipid metabolism pathway, pertussis pathway, phagosome pathway, phase 1-functionalization of compounds pathway, phase I biotransformations pathway, phase 11 conjugation pathway, phenylacetate degradation pathway, phenylalanine biosynthesis pathway, phenylalanine metabolism pathway, phenylethylamine degradation pathway, phenylpropionate degradation pathway, phosphatidylinositol signaling system pathway, phospholipase D signaling pathway, phototransduction pathway, PI3 kinase pathway, PI3K signaling in B-lymphocytes pathway, PI3K-AKT signaling pathway, PIP3 activates AKT signaling pathway, plasminogen activating cascade pathway, platelet activation pathway, platelet adhesion to exposed collagen pathway, platelet aggregation pathway, platelet homeostasis pathway, polyol pathway, porphyrin and chlorophyll metabolism pathway, PPAR signaling pathway, primary bile acid biosynthesis pathway, primary focal segmental glomerulosclerosis FSGs pathway, processing of capped intron-containing pre-mRNA pathway, processing of capped intronless pre-mRNA pathway, progesterone-mediated oocyte maturation pathway, prolactin signaling pathway, proline biosynthesis pathway, propanoate metabolism pathway, prostaglandin synthesis and regulation pathway, proteasome pathway, proteasome degradation pathway, protein digestion and absorption pathway, protein export pathway, protein folding pathway, proximal tubule bicarbonate reclamation pathway, PRPP biosynthesis pathway, PTEN pathway, purine metabolism pathway, pyridoxal phosphate salvage pathway, pyridoxal-5-phosphate biosynthesis pathway, pyrimidine metabolism pathway, pyruvate metabolism pathway, racl pathway, rank signaling in osteoclast pathway, rank)/rank pathway, rapl signaling pathway, ras signaling pathway, Ras-RAF-MEK-ERK pathway, receptor activator of nuclear factor kappa-b ligand (RANKL) pathway, regulation of actin cytoskeleton pathway, regulation of apoptosis pathway, regulation of autophagy pathway, regulation of DNA replication pathway, regulation of lipolysis in adipocytes pathway, regulation of microtubule cytoskeleton pathway, regulation of toll-like receptor signaling pathway, remodeling of adherens junctions pathway, renin secretion pathway, renin-angiotensin system pathway, respiratory electron transport pathway, retinol metabolism pathway, retrograde endocannabinoid signaling pathway, Rho family GTPase pathway, Rhoa pathway, ribosome biogenesis in eukaryotes pathway, RIG-I-like receptor signaling pathway, RNA degradation pathway, RNA polymerase 1 pathway, RNA polymerase II transcription pathway, RNA transport pathway, RNAi pathway, s-adenosylmethionine biosynthesis pathway, salivary secretion pathway, salvage pyrimidine deoxyribonucleotides pathway, salvage pyrimidine ribonucleotides pathway, SCW signaling pathway, selenium metabolism and selenoproteins pathway, selenium micronutrient network pathway, selenocompound metabolism pathway, semaphorin interactions pathway, serine and threonine metabolism pathway, serine glycine biosynthesis pathway, serotonergic synapse pathway, serotonin htr1 group and fos pathway, serotonin receptor 2 and ELK-SRF/gata4 signaling pathway, serotonin receptor 2 and STAT3 signaling pathway, serotonin receptor 4/6/7 and NR3C signaling pathway, serotonin transporter activity pathway, signal amplification pathway, signal regulatory protein pathway, signal transduction of S1P receptor pathway, signaling by EGFR pathway, signaling by insulin receptor pathway, signaling by PDGF pathway, signaling by rho GTPases pathway, signaling by robo receptor pathway, signaling by VEGF pathway, signaling in gap junction pathway, signaling of hepatocyte growth factor receptor pathway, signaling regulating pluripotency of stem cells pathway, signaling in glioblastoma pathway, signaling by NGF pathway, SMAD signaling network pathway, small ligand GPCRs pathway, SNARE interactions in vesicular transport pathway, sphingolipid (SM) signaling pathway, sphingolipid metabolism pathway, spliceosome pathway, starch and sucrose metabolism pathway, stat signaling pathway, STAT3 pathway, statin pathway, steroid biosynthesis pathway, steroid hormone biosynthesis pathway, sterol regulatory element-binding proteins pathway, striated muscle contraction pathway, succinate to proprionate conversion pathway, sulfate assimilation pathway, sulfation biotransformation reaction pathway, sulfur metabolism pathway, sulfur relay system pathway, sumo pathway, synaptic vesicle pathway, synthesis and degradation of ketone bodies pathway, synthesis of DNA pathway, T cell receptor (TCR) pathway, tamoxifen metabolism pathway, tarbase pathway, target of rapamycin pathway, taste transduction pathway, taurine and hypotaurine metabolism pathway, TCA and urea cycles pathway, T-cell antigen receptor pathway, T-cell receptor and co-stimulatory signaling pathway, telomere maintenance pathway, terpenoid backbone biosynthesis pathway, tetrahydrofolate biosynthesis pathway, TFS regulate miRNAs related to cardiac hypertrophy pathway, TGF-beta pathway, TGF-beta receptor signaling pathway, THC differentiation pathway, thiamin biosynthesis pathway, thiamin metabolism pathway, threonine biosynthesis pathway, thymic stromal lymphopoietin pathway, thymic stromal lymphopoietin (tslp) pathway, thyroid-stimulating hormone (tsh) pathway, thyrotropin-releasing hormone receptor signaling pathway, tie2/tek signaling pathway, tight junction pathway, TNF alpha signaling pathway, TNF related weak inducer of apoptosis pathway, TNF signaling pathway, TNF superfamily pathway, TNF-related weak inducer of apoptosis (tweak) pathway, toll receptor signaling pathway, toll-like receptors pathway, TP53 network pathway, Traf pathway, trail pathway, transcription regulation by bzip transcription factor pathway, transcriptional activation by Nrf2 pathway, transendothelial migration of leukocytes pathway, transforming growth factor beta (TGF-beta) receptor pathway, translation factors pathway, transmission across electrical synapses pathway, transport of glucose and other sugars pathway, transport of glycerol from adipocytes to the liver by aquaporins pathway, transport of vitamins pathway, trans-sulfuration pathway, trans-sulfuration and one carbon metabolism pathway, triacylglyceride synthesis pathway, triacylglycerol metabolism pathway, tRNA aminoacylation pathway, tryptophan biosynthesis pathway, tryptophan metabolism pathway, tumor necrosis factor (TNF) alpha pathway, tumoricidal effects of hepatic NK cells pathway, tweak pathway, type H diabetes mellitus pathway, type H interferon signaling pathway, type III interferon signaling pathway, tyrosine and tryptophan biosynthesis pathway, tyrosine biosynthesis pathway, tyrosine metabolism pathway, ubiquinone and other terpenoid-quinone biosynthesis pathway, ubiquitin mediated proteolysis pathway, ubiquitin proteasome pathway, unfolded protein response pathway, urea cycle and metabolism of amino groups pathway, valine biosynthesis pathway, vascular smooth muscle contraction pathway, vasopressin synthesis pathway, vasopressin-regulated water reabsorption pathway, VEGF signaling pathway, vitamin a and carotenoid metabolism pathway, vitamin b12 metabolism pathway, vitamin b6 biosynthesis pathway, vitamin b6 metabolism pathway, vitamin d metabolism pathway, vitamin digestion and absorption pathway, Wnt signaling pathway, xanthine and guanine salvage pathway, and/or the zinc homeostasis pathway

### Genome Editing Approaches

In certain embodiments, altering the chromosomal regions defining INs and their GSCs and genes may be used to treat disease by modulating the production of gene products, such as activating a GSC to increase production of a gene product, eliminating a gene product or targeting any one of the members of the molecules of the GSN or networks associated with the IN. In some embodiments, expression of a wild-type allele may be boosted in a patient suffering from a haploinsufficiency. Similarly, modulation could benefit other types of genetically-based insufficiencies, including those caused by other hetero- and homozygous errors in ORFs and regulatory genetic sequences that control ORF expression.

Methods of altering the gene expression attendant to an insulated neighborhood include altering the boundaries of the insulated neighborhood or a gene loop and/or the binding site(s) of a genomic signaling center, for example, using CRISPR to make alterations or repair/replace if mutated). In some embodiments, the genome editing tool targets at least one occupancy-dependent signaling center including a nucleic acid sequence comprising SEQ ID NOs: 32,627-71,281. These alterations may results in a variety a results including: activation of cell death pathways prematurely/inappropriately (key to many immune disorders), production of too little/much gene product (also known as the rheostat hypothesis), production of too little/much extracellular secretion of enzymes, prevention lineage differentiation, switch of lineage pathways, promotion of stemness, initiation or interference with auto regulatory feedback loops, initiation of errors in cell metabolism, inappropriate imprinting/gene silencing, and formation flawed chromatin states. Additionally, genome editing approaches well-known in the art to create new boundaries for INs or binding sites of GSCs by altering the cohesin necklace or moving genes and enhancers.

In certain embodiments, genome editing approaches describe herein may include methods of using site-specific nucleases to introduce single-strand or double-strand DNA breaks at particular locations within the genome. Such breaks can be and regularly are repaired by endogenous cellular processes, such as homology-directed repair (HDR) and non-homologous end joining (NHEJ). HDR is essentially an error-free mechanism that repairs double-strand DNA breaks in the presence of a homologous DNA sequence. The most common form of HDR is homologous recombination. It utilizes a homologous sequence as a template for inserting or replacing a specific DNA sequence at the break point. The template for the homologous DNA sequence can be an endogenous sequence (e.g., a sister chromatid), or an exogenous or supplied sequence (e.g., plasmid or an oligonucleotide). As such, HDR may be utilized to introduce precise alterations such as replacement or insertion at desired regions. In contrast, NHEJ is an error-prone repair mechanism that directly joins the DNA ends resulting from a double-strand break with the possibility of losing, adding or mutating a few nucleotides at the cleavage site. The resulting small deletions or insertions (termed “Indels”) or mutations may disrupt or enhance gene expression. Additionally, if there are two breaks on the same DNA, NHEJ can lead to the deletion or inversion of the intervening segment. Therefore, NHEJ may be utilized to introduce insertions, deletions or mutations at the cleavage site.

### CRISPR

In certain embodiments, a CRISPR/Cas system may be used to delete CTCF anchor sites to modulate gene expression within the IN associated with that anchor site. See, Hnisz et al., Cell 167, Nov. 17, 2016, which is hereby incorporated by reference in its entirety. Disruption of the boundaries of IN prevents the interactions necessary for proper function of the associated GSCs. Changes in the expression genes that are immediately adjacent to the deleted neighborhood boundary have also been observed due to such disruptions.

In certain embodiments, a CRISPR/Cas system may be used to modify existing CTCF anchor sites. For example, existing CTCF anchor sites may be mutated or inverted by inducing NHEJ with a CRISPR/Cas nuclease and one or more guide RNAs, or masked by targeted binding with a catalytically inactive CRISPR/Cas enzyme and one or more guide RNAs. Alteration of existing CTCF anchor sites may disrupt the formation of existing INs and after the expression of genes located within these INs.

In certain embodiments, a CRISPR/Cas system may be used to introduce new CTCF anchor sites. CTCF anchor sites may be introduced by inducing HDR at a selected site with a CRISPR/Cas nuclease, one or more guide RNAs and a donor template containing the sequence of a CTCF anchor site. Introduction of new CTCF anchor sites may create new INs and/or after existing INs, which may affect expression of genes that are located adjacent to these INs.

In certain embodiments, a CRISPR/Cas system may be used to after the binding sites of a GSC. For example, if the GSC contains a mutation that affects the assembly at the region with associated transcription factors, the mutated site may be repaired by inducing a double strand DNA break at or near the mutation using a CRISPR/Cas nuclease and one or more guide RNAs in the presence of a supplied corrected donor template.

In certain embodiments, a CRISPR/Cas system may be used to modulate expression of neighborhood genes by binding to a region within an IN (e.g., enhancer) and block transcription. Such binding may prevent recruitment of transcription factors to GSCs and initiation of transcription. The CRISPR/Cas system may be a catalytically inactive CRISPR/Cas system that do not cleave DNA.

In certain embodiments, a CRISPR/Cas system may be used to knockdown expression of neighborhood genes via introduction of short deletions in coding regions of these genes. When repaired, such deletions would result in frame shifts and/or introduce premature stop codons in mRNA produced by the genes followed by the mRNA degradation via nonsense-mediated decay. In other embodiments, a CRISPR/Cas system may also be used to after cohesion necklace or moving genes and enhancers.

In certain embodiments, a CRISPR/Cas system may be used to perturb an ODSC. For example, the CRISPR/Cas system may target at least one of SEQ ID NOs: 32,627-71,281.

### CRISPR/Cas Enzymes

CRISPR/Cas systems are bacterial adaptive immune systems that utilize RNA-guided endonucleases to target specific sequences and degrade target nucleic acids. They have been adapted for use in various applications in the field of genome editing and/or transcription modulation. Any of the enzymes or orthologs known in the art or disclosed herein may be utilized in the methods herein for genome editing.

In certain embodiments, the CRISPR/Cas system may be a Type II CRISPR/Cas9 system. Cas9 is an endonuclease that functions together with a trans-activating CRISPR RNA (tracrRNA) and a CRISPR RNA (crRNA) to cleave double stranded DNAs. The two RNAs can be engineered to form a single-molecule guide RNA by connecting the 3′ end of the crRNA to the 5′ end of tracrRNA with a linker loop. Jinek et al., Science, 337(6096):816-821 (2012) showed that the CRISPR/Cas9 system is useful for RNA-programmable genome editing, and international patent application WO 2013/176772 provides numerous examples and applications of the CRISPR/Cas endonuclease system for site-specific gene editing, which are incorporated herein by reference in their entirety. Exemplary CRISPR/Cas9 systems include those derived from Streptococcus pyogenes, Streptococcus thermophilus, Neisseria meningitidis, Treponema denticola, Streptococcus aureas, and Francisella tularensis.

In certain embodiments, the CRISPR/Cas system may be a Type V CRISPR/Cpf1 system. Cpf1 is a single RNA-guided endonuclease that, in contrast to Type II systems, lacks tracrRNA. Cpf1 produces staggered DNA double-stranded break with a 4 or 5 nucleotide 5′ overhang. Zetsche et al. Cell. 2015 Oct. 22; 163(3):759-71 provides examples of Cpf1 endonuclease that can be used in genome editing applications, which is incorporated herein by reference in its entirety. Exemplary CRISPR/Cpf1 systems include those derived from Francisella tularensis, Acidaminococcus sp., and Lachnospiraceae bacterium.

In certain embodiments, nickase variants of the CRISPR/Cas endonucleases that have one or the other nuclease domain inactivated may be used to increase the specificity of CRISPR-mediated genome editing. Nickases have been shown to promote HDR versus NHEJ. HDR can be directed from individual Cas nickases or using pairs of nickases that flank the target area.

In certain embodiments, catalytically inactive CRISPR/Cas systems may be used to bind to target regions (e.g., CTCF anchor sites or enhancers) and interfere with their function. Cas nucleases such as Cas9 and Cpf1 encompass two nuclease domains. Mutating critical residues at the catalytic sites creates variants that only bind to target sites but do not result in cleavage. Binding to chromosomal regions (e.g., CTCF anchor sites or enhancers) may disrupt proper formation of INs or GSCs and therefore lead to altered expression of genes located adjacent to the target region. For example, an ODSC is targeted by a catalytically inactive CRISPR/Cas system and thereby disrupted.

In certain embodiments, a CRISPR/Cas system may include additional functional domain(s) fused to the CRISPR/Cas endonuclease or enzyme. The functional domains may be involved in processes including but not limited to transcription activation, transcription repression, DNA methylation, histone modification, and/or chromatin remodeling. Such functional domains include but are not limited to a transcriptional activation domain (e.g., VP64 or KRAB, SID or SID4×), a transcriptional repressor, a recombinase, a transposase, a histone remodeler, a DNA methyltransferase, a cryptochrome, a light inducible/controllable domain or a chemically inducible/controllable domain.

In certain embodiments, a CRISPR/Cas endonuclease or enzyme may be administered to a cell or a patient as one or a combination of the following: one or more polypeptides, one or more mRNAs encoding the polypeptide, or one or more DNAs encoding the polypeptide.

### Guide Nucleic Acid

In certain embodiments, guide nucleic acids may be used to direct the activities of an associated CRISPR/Cas enzymes to a specific target sequence within a target nucleic acid. Guide nucleic acids provide target specificity to the guide nucleic acid and CRISPR/Cas complexes by virtue of their association with the CRISPR/Cas enzymes, and the guide nucleic acids thus can direct the activity of the CRISPR/Cas enzymes.

In one aspect, guide nucleic acids may be RNA molecules. In one aspect, guide RNAs may be single-molecule guide RNAs. In one aspect, guide RNAs may be chemically modified.

In certain embodiments, more than one guide RNAs may be provided to mediate multiple CRISPR/Cas-mediated activities at different sites within the genome.

In certain embodiments, guide RNAs may be administered to a cell or a patient as one or more RNA molecules or one or more DNAs encoding the RNA sequences. In certain embodiments, a guide RNA used herein targets at least a portion of a GSC, for example, an ODSC comprising a nucleic acid sequence of one of SEQ ID NOs: 32,627-71,281.

### Ribonucleoprotein Complexes (RNPs)

In one embodiment, the CRISPR/Cas enzyme and guide nucleic acid may each be administered separately to a cell or a patient. In another embodiment, the CRISPR/Cas enzyme may be pre-complexed with one or more guide nucleic acids. The pre-complexed material may then be administered to a cell or a patient. Such pre-complexed material is known as a ribonucleoprotein particle (RNP).

### Zinc Finger Nucleases

In certain embodiments, genome editing approaches of the present invention involve the use of Zinc finger nucleases (ZFNs). ZFNs are modular proteins comprised of an engineered zinc finger DNA binding domain linked to a DNA-cleavage domain. A typical DNA-cleavage domain is the catalytic domain of the type II endonuclease FokI. Because FokI functions only as a dimer, a pair of ZFNs are engineered to bind to cognate target “haft-site” sequences on opposite DNA strands and with precise spacing between them to enable the catalytically active FokI domains to dimerize. Upon dimerization of the FokI domain, which itself has no sequence specificity per se, a DNA double-strand break is generated between the ZFN haft-sites as the initiating step in genome editing. Transcription Activator-Like Effector Nucleases (TALENs)

In certain embodiments, genome editing approaches of the present invention involve the use of Transcription Activator-Like Effector Nucleases (TALENs). TALENs represent another format of modular nucleases which, similarly to ZFNs, are generated by fusing an engineered DNA binding domain to a nuclease domain, and operate in tandem to achieve targeted DNA cleavage. While the DNA binding domain in ZFN consists of zinc finger motifs, the TALEN DNA binding domain is derived from transcription activator-like effector (TALE) proteins, which were originally described in the plant bacterial pathogen Xanthomonas sp. TALEs are comprised of tandem arrays of 33-35 amino acid repeats, with each repeat recognizing a single basepair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

### Other Site-Specific Nucleases

In certain embodiments, genome editing approaches of the present invention involve the use of homing endonucleases (HEs). HEs are a class of sequence-specific endonucleases that have long recognition sequences and cleave DNA with high specificity. Homing endonuclease recognition sequences are long enough (14-44 base pairs) to occur randomly only with a very low probability. Currently there are at least six known structural families of HEs, including GIY-YIG, His-Cis box, H—N—H, PD-(D/E)xK, and Vsr-like. These HEs are derived from a broad range of hosts, including eukarya, protists, bacteria, archaea, cyanobacteria and phage. HEs can be used to create a DSB at a target locus as the initial step in genome editing similarly to ZFNs and TALENs. Additionally, some natural and engineered HEs cut only a single strand of DNA, thereby functioning as site-specific nickases.

In further embodiments, genome editing approaches of the present invention involve the use of a variant or a hybrid of the site-specific nucleases described herein, such as MegaTAL/Tev-mTALEN/MegaTev, dCas9-FokI, dCpf1-Fok1 or other nucleases as described in US patent publication 20180021413A1, which is hereby incorporated by reference in its entirety.

### Hybridizing Oligonucleotides

In some embodiments, oligonucleotides, including those which function via a hybridization mechanism, whether single of double stranded such as antisense molecules, RNAi constructs (including siRNA, saRNA, microRNA, etc.), aptamers and ribozymes may be used to after or as perturbation stimuli of the targeted systems of the invention.

As such oligonucleotides may also serve as therapeutics, their therapeutic liabilities and treatment outcomes may be ameliorated or predicted, respectively by interrogating the gene signaling networks of the invention. In some embodiments, these stimuli bind a genomic signaling center. In certain embodiments, the oligonucleotide hybridizes to at least a portion of an occupancy-dependent signaling center comprising a nucleic acid sequence of SEQ ID NOs: 32,627-71,281.

### Gene Expression Modulation

In certain embodiments, perturbation of the systems using at least one stimuli described above results in modulation of gene expression. For example, methods of altering expression of at least one gene selected from any of those in Tables 1-9 with at least one compound selected from those described herein or any others are described herein. In certain embodiments, the gene may be one expressed in liver cells. Alternatively, the gene may be expressed in erythroid cells.

### Alteration of Neighborhood Structures

Human cell types share the same IN boundaries, although the location and composition of super enhancers is cell type dependent, as described in Hnisz et al., Cell 167, Nov. 17, 2016, which is hereby incorporated by reference in its entirety. Since a majority of RNA Polymerase II-associated interactions occur within an IN, changing the boundaries of an IN will result in inappropriate enhancer-gene interactions and genomic signaling center function, thereby leading to misregulation of local genes. In certain embodiments, at least one perturbation stimuli described herein is applied to a genomic system to after the boundaries of an IN and disrupt GSCs. In some embodiments, disruption of GSCs results in a change in the binding profile. In certain embodiments, alteration of IN structure results in disruption of an ODSC.

### Mutation/Deletion of CTCF Sites

One element that determines the boundary of an IN is the CTCF-CTCF loop anchors of INs, which have been observed to play important roles in gene regulation, which is described in Hnisz et al., Cell 167, Nov. 17, 2016 and Hnisz et al., Science Reports, 351(6380):1454-1458 (2016), which are hereby incorporated by reference in their entireties. The importance of these sites is supported by the fact that genetic variants rarely occur in CTCF-CTCF anchor regions. Mutations of the anchor sites likely results in disruption of the IN and the associated GSCs. In certain embodiments, at least one genome editing approach described herein is used to mutate or delete CTCF anchor sites to modulate expression of at least one gene in Tables 1-9.

### Methylation/Demethylation

In certain embodiments, a method of modulating expression of at least one gene in Tables 1-9 includes administering at least one stimuli described herein to after the methylation pattern of DNA. Methylation and demethylation of promoters is important for maintenance of cell-type specific transcriptional programs and regulation of gene expression that modulates phenotype in cells, as described in Stepper et al., Nucleic Acids Research, 2016, di 10.1093/nalgkwl1112, 1-11, which is hereby incorporated by reference in its entirety. DNA methylation has been observed to be a key mechanism in cellular reprogramming processes, including differentiation, senescence, and disease. Further, CTCF has been observed to preferentially bind to hypomethylated DNA, as described in Ji et al., 2016, Cell Stem Cell, 18, 262-275, which is hereby incorporated by reference in its entirety. Mutations resulting in misregulation of the methylation machinery is common in many cancers because methylation is associated with transcriptional silencing. Improper demethylation near a promoter region may result in a change of the binding profile of a GSC leading to a distortion of the chromatin structure including displacement of nucleosomes, thereby preventing proper function of the GSC. In certain embodiments, improper demethylation near a promoter region results in a change of the binding profile of an ODSC.

### Rescue of Disease Phenotype

GSCs or INs may be modulated to treat any one of the genetic disease associated with genetic mutations. For example, Hemophilia B, also called factor IX deficiency, is caused by reduced production of functional factor IX in the liver. In certain embodiments, at least one stimulus selected from those described herein may be administered to modulate a GSC that controls factor IX expression to increase androgen receptor binding resulting in an increased level of the functional factor IX, whose deficiency causes the phenotype.

A coding region mutation is also responsible for glycogen storage disease 1b. The mutation results in a partially functional protein. At least one stimulus selected from those described herein that targets the GSC responsible for increasing, although not completely rescuing the level of the protein, may be sufficient to rescue the phenotype of the disease.

Cancers with Therapeutic Potential

In certain embodiments, a method of modulating expression of at least one gene in Tables 1-9 includes administering at least one stimuli from those described herein to restore the boundary of at least one IN to prevent formation of a gene loop having a GSC that controls expression of a proto-oncogene. The regions of DNA near proto-oncogenes frequently include mutations in IN boundaries that bring transcriptional enhancers into proximity of these genes, as described in Hnisz et al., Science Reports, 351(6280):1454-1458 (2016), which is hereby incorporated by reference herein in its entirety. In fact, previous studies have shown that some INs function to prevent proto-oncogene activation. Even site-specific perturbation of an IN boundary has been shown to result in activation a proto-oncogene in the LMO2 locus. LMO2 encodes a transcription factor that is considered oncogenic when overexpressed. In some embodiments, perturbation of the boundaries of an IN alters the binding profile of a GSC. For example, the perturbation alters the binding profile of an ODSC.

### Treatment of Diseases

In certain embodiments, modulating GSCs or INs associated with at least one gene in Tables 1-9 may be used for treating a disease selected from Disease/Condition, Abdominal abscess, Abdominal aortic aneurysm, Abdominal cavity inflammation, Abdominal pain, Abdominal surgery, Abnormal labor, Abortion, Abscess, Absence seizure, Achalasia, Acid base imbalance, Acidemia, Acidosis, Acinetobacter baumanii infection, Acne, Acne vulgaris, Acoustic neuroma, Acquired immune deficiency syndrome, Acral erythema, Acromegaly, Actinic keratosis, Acute bronchitis, Acute chest syndrome, Acute coronary syndrome, Acute decompensated heart failure, Acute external otitis, Acute leukemia, Acute liver failure, Acute lung injury, Acute lymphoblastic leukemia, Acute myelogenous leukemia, Acute promyelocytic leukemia, Acute sinusitis, Acute stress disorder, Addisons disease, Adenocarcinoma, Adenoid hypertrophy, Adenoid tumor, Adenoma, Adenomyosis, Adenosine deaminase deficiency, Adenovirus infection, Adhesive capsulitis, Adipose tissue neoplasm, Adrenal cortex disease, Adrenal cortex neoplasm, Adrenal cortical adenoma, Adrenal cortical carcinoma, Adrenal disease, Adrenal gland hypofunction, Adrenal gland tumor, Adrenoleukodystrophy, Adrenomyeloneuropathy, Adult onset Stills disease, Adult respiratory distress syndrome, Adult T-cell lymphoma, Adult varicella zoster virus infection, Advanced solid tumor, Advanced solid tumor, Nasopharyngeal carcinoma, Agammaglobulinemia, Age related macular degeneration, Aggression, Aggressive fibromatosis, Aging, Agitation, Agoraphobia, AIDS related complex, Ar sickness, Akathesia, AL amyloidosis, Alagille syndrome, Albinism, Albuminuria, Alcohol overdose, Alcohol withdrawal delirium, Alcohol withdrawal syndrome, Alcoholic fatty liver disease, Alcoholic hepatitis, Alcoholic liver cirrhosis, Alcoholic liver disease, Alcoholism, Alkalosis, Alkaptonuria, Allergic conjunctivitis, Allergic rhinitis, Allergy, Alopecia, Alopecia areata, Alopecia drug-induced, Alpha mannosidosis, Alpha-1 antitrypsin deficiency, Alport syndrome, Altitude anoxia, Alveolar bone loss, Alzheimers disease, Amblyopia, Amenorrhea, Amino acid and protein metabolism disorder, Amino acid metabolism disorder, Amnesia, Amoeba infection, Amphetamine dependence, Amyloid polyneuropathy, Amyloidosis, Anal disease, Anal dysplasia, Anal fissure, Anal fistula, Anal tumor, Anaplastic astrocytoma, Anaplastic large cell lymphoma, Anaplastic thyroid cancer, Androgen deficiency, Androgen insensitivity syndrome, Anemia, Anesthesia, Aneuploidy, Aneurysm, Angelman syndrome, Angina, Angiodermatitis, Angiodysplasia, Angioedema, Angiogenesis disorder, Angioimmunoblastic T-cell lymphoma, Angiomyolipoma, Angiosarcoma, Angle closure glaucoma, Anhidrosis, Anisometropia, Ankylosing spondylitis, Anorectal disease, Anorexia nervosa, Anovulation, Anticoagulant overdose, Antiphospholipid syndrome, Anus pain, Anxiety disorder, Aortic aneurysm, Aortic disease, Aortic stenosis, Aortic valve disease, Aortic valve insufficiency, Aortic valve stenosis, Aphasia, Aplasia, Aplastic anemia, Apnea, Appendicitis, Appetite loss, Apraxia, Arachnid infection, Arachnoiditis, Arterial hypertension, Arterial occlusive disease, Arterial thrombosis, Arteriosclerosis, Arteriovenous malformation, Artery disease, Artery injury, Arthralgia, Arthritis, Asbestosis, Ascites, Aseptic meningitis, Asperger syndrome, Aspergillus fumigatus infection, Aspergillus infection, Asphyxia, Asplenia, Asthenozoospermia, Asthma, Asthma attack, Astigmatism, Astrocytoma, Ataxia, Ataxia telangiectasia, Atelectasis, Atherosclerosis, Atopic dermatitis, Atrial fibrillation, Atrial flutter, Atrioventricular block, Atrophic vaginitis, Atrophy, Attention deficit hyperactivity disorder, Attention deficit-disruptive behavior disorder, Autism, Autoimmune disease, Autoimmune hemolytic anemia, Autoimmune hepatitis, Autoinflammatory disease, Autonomic nervous system disease, Autosomal disorder, Autosomal dominant disorder, Autosomal dominant polycystic kidney disease, Azoospermia, Bacilli infection, Bacillus anthracis infection, Back pain, Bacterial endocarditis, Bacterial eye infection, Bacterial infection, Bacterial meningitis, Bacterial pneumonia, Bacterial respiratory tract infection, Bacterial skin infection, Bacterial urinary tract infection, Bacterial vaginosis, Bacteriuria, Bacteroides fragilis infection, Bacteroides infection, Bacteroides thetaiotaomicron infection, Balanitis, Barretts esophagus, Bartonella bacilliformis infection, Basal cell carcinoma, Basal cell nevus syndrome, Bassen Kornsweig syndrome, B-cell acute lymphoblastic leukemia, B-cell lymphoma, Becker muscular dystrophy, Behcets disease, Bells palsy, Benign skin tumor, Benign tumor, Berylliosis, Beta thalassemia, Beta-hemolytic Streptococcus infection, Biliary atresia, Biliary cancer, Biliary cirrhosis, Biliary tract disease, Biliary tumor, Binge eating disorder, Bipolar disorder, Bipolar I disorder, Bipolar H disorder, Birt Hogg Dube syndrome, Birth complication, BK virus infection, Bladder cancer, Bladder disease, Bladder pain, Bladder tumor, Bleeding, Blepharitis, Blepharoconjunctivitis, Blepharospasm, Blindness, Blood clotting disorder, Blood clotting factor deficiency, Blood transfusion, Body dysmorphic syndrome, Body weight change, Bone and joint infection, Bone disease, Bone injury, Bone marrow aplasia, Bone marrow depression, Bone marrow disease, Bone marrow failure, Bone marrow transplant rejection, Bone marrow transplantation, Bone metastases, Bone resorption, Bone tumor, Borderline personality disorder, Bordetella pertussis infection, Borrelia burgdorferi infection, Borrelia infection, Bowen disease, Bradycardia, Brain disease, Brain hemorrhage, Brain hypoxia-ischemia, Brain infarction, Brain injury, Brain ischemia, Brain stem infarction, Brain tumor, Breast Cancer, Breast disease, Breast tumor, Brenner tumor, Bronchiectasis, Bronchiolitis, Bronchitis, Bronchospasm, Bronchus disease, Brucella infection, Brugada syndrome, Bruxism, Bulimia nervosa, Bullous pemphigoid, Burkitts lymphoma, Bursitis, Cachexia, Calcification, Calcinosis, Calcium metabolism disorder, Campylobacter infection, Campylobacter jejuni infection, Canavan disease, Cancer, Cancer pain, Candida albicans infection, Candida infection, Candida krusei infection, Cannabis dependence, Capillary disease, Carbamoyl phosphate synthase deficiency, Carbohydrate metabolism disorder, Carcinoid syndrome, Carcinoid tumor, Carcinoma, Carcinosarcoma, Cardiac arrest, Cardiac edema, Cardiac failure, Cardiac hypertrophy, Cardiac reperfusion injury, Cardiac surgical procedure, Cardialgia, Cardiogenic shock, Cardioplegia, Cardiopulmonary bypass surgery, Cardiotoxicity drug-induced, Cardiovascular development disorder, Cardiovascular disease, Cardiovascular inflammation, Cardiovascular injury, Cardiovascular pregnancy complications, Cardiovascular surgical procedure, Cardiovascular symptom, Carditis, Carnitine palmitoyltransferase 2 deficiency, Carotid sinus syndrome, Carotid stenosis, Carpal tunnel syndrome, Carthage disease, Castlemans disease, Cataplexy, Cataract, Catatonic schizophrenia, Causalgia, Celiac disease, Cell transplantation, Cellulitis, Central nervous system disease, Central nervous system tumor, Cerebellar ataxia, Cerebral amyloid angiopathy, Cerebral edema, Cerebral hemorrhage, Cerebral hypoxia, Cerebral infarction, Cerebral palsy, Cerebrotendinous xanthomatosis, Cerebrovascular disease, Cerebrovascular trauma, Cervical dysplasia, Cervical dystonia, Cervicitis, Cervix disease, Cervix infection, Cervix injury, Chagas cardiomyopathy, Chalazion, Charcot Marie Tooth disease, Charcot Marie Tooth type 1 disease, Cheilitis, Chemotherapy induced nausea and vomiting, Chemotherapy-induced emesis, Cheynes Stokes breathing, Chikungunya virus infection, Chills, Chlamydia infection, Chlamydia pneumoniae infection, Chlamydia trachomatis infection, Chlamydiales infection, Cholangiocarcinoma, Cholangitis, Cholecystitis, Cholelithiasis, Cholestasis, Cholesterol metabolism disorder, Chondrocalcinosis, Chondrosarcoma, Chordoma, Chorea, Choriocarcinoma, Chorioretinitis, Choroid disease, Choroidal neovascularization, Choroideremia, Choroiditis, Chromoblastomycosis, Chromosome aberration, Chromosome disorder, Chronic bronchitis, Chronic fatigue syndrome, Chronic granulomatous disease, Chronic inflammatory demyelinating polyneuropathy, Chronic leukemia, Chronic liver failure, Chronic lymphocytic leukemia, Chronic myelocytic leukemia, Chronic myelomonocytic leukemia, Chronic obstructive airway disease, Chronic obstructive pulmonary disease, Chronic sinusitis, Chronic thromboembolic pulmonary hypertension, Churg-Strauss syndrome, Chylothorax, CINCA syndrome, Circadian rhythm sleep disorder, Citrobacter infection, Citrullinuria, Cleft hp, Cleft palate, Climacteric disorder, Clostridium botulinum infection, Clostridium difficile infection, Clostridium infection, Clostridium tetani infection, Cluster headache, CMV retinitis, Cocaine addiction, Coccidioides infection, Cockayne syndrome, Cognitive disorder, Cold agglutinin disease, Colic, Colitis, Colon disease, Colon tumor, Colonoscopy, Colorectal tumor, Combined immunodeficiency, Common cold, Common variable immunodeficiency, Compensated liver cirrhosis, Complex partial seizure, Complex regional pain syndrome, Complicated skin and skin structure infection, Complicated urinary tract infection, Compulsive gambling, Conduct disorder, Condyloma, Confusion, Congenital adrenal hyperplasia, Congenital autonomic control failure, Congenital heart defect, Congenital hemophilia, Congenital ichthyosiform erythroderma, Congenital lung disease, Congestive cardiomyopathy, Congestive heart failure, Conjunctiva disease, Conjunctivitis, Connective tissue disease, Connective tissue disease affecting the skin, Consciousness disorder, Constipation, Constipation predominant irritable bowel syndrome, Contact dermatitis, Contraception, Contracture, Contusion, Conversion disorder, Copper metabolism disorder, Corneal disease, Corneal injury, Corneal pain, Corneal transplant rejection, Corneal transplantation, Corneal ulcer, Coronary arteriosclerosis, Coronary artery disease, Coronary atherosclerosis, Coronary thrombosis, Coronary vasospasm, Corynebacterium diphtheriae infection, Cough, Coxiella infection, Coxsackie virus infection, Cranial nerve tumor, Craniofacial development disorder, Craniosynostosis, Creutzfeldt Jakob disease, Crigler-Najjar syndrome, Crohns disease, Croup, Cryoglobulinemia, Cryopyrin associated periodic syndrome, Cryptococcus infection, Cryptococcus neoformans meningitis, Cryptosporidium infection, Cushings disease, Cushings syndrome, Cutaneous lupus erythematosus, Cutaneous T-cell lymphoma, Cyst, Cystic fibrosis, Cysticercosis, Cystinosis, Cystinuria, Cystitis, Cytomegalovirus infection, Dacryocystitis, Death, Decompensated liver cirrhosis, Decubitus ulcer, Deep vein thrombosis, Degenerative disc disease, Delayed hypersensitivity, Delayed puberty, Delirium, Deltavirus infection, Delusional disorder, Dementia, Demyelinating disease, Dengue virus infection, Dental caries, Dental pulp disease, Dents disease, Depersonalization disorder, Depression, Dermatitis, Dermatofibrosarcoma, Dermatological disease, Dermatomycosis, Dermatomyositis, Dermatophytosis, Diabetes insipidus, Diabetes mellitus, Diabetic angiopathy, Diabetic cardiomyopathy, Diabetic cataract, Diabetic complication, Diabetic foot ulcer, Diabetic gastroparesis, Diabetic hypertension, Diabetic ketoacidosis, Diabetic macular edema, Diabetic nephropathy, Diabetic neuropathy, Diabetic peripheral neuropathy, Diabetic retinopathy, Diamond Blackfan anemia, Diarrhea, Diarrhea predominant irritable bowel syndrome, Diastolic heart failure, Diffuse large B-cell lymphoma, Dilated cardiomyopathy, Diplegia, Discoid lupus erythematosus, Disseminated intravascular coagulation, Dissociative disorder, Diverticulitis, Diverticulosis, Dizziness, DNA virus infection, Down syndrome, Drowsiness, Drug dependence, Drug overdose, Drug withdrawal syndrome, Dry age related macular degeneration, Duchenne dystrophy, Dumping syndrome, Duodenal ulcer, Dupuytren contracture, Dwarfism, Dyschezia, Dyskeratosis congenita, Dyslexia, Dysmenorrhea, Dyspareunia, Dyspepsia, Dysphagia, Dysphoria, Dysplasia, Dysplastic nevus syndrome, Dyspnea, Dysthymic disorder, Dystonia, Dystrophy, Dysuria, Eale disease, Ear disease, Eating disorder, Ebola virus infection, Eclampsia, Ectopic pregnancy, Eczema, Edema, Egg hypersensitivity, Ehlers Danlos syndrome, Eisenmenger syndrome, Electrolyte metabolism disorder, Elephantiasis, Embolism, Embolism and thrombosis, Emesis, Emotional lability, Emphysema, Empyema, Encephalitis, End stage renal disease, Endocarditis, Endocrine disease, Endocrine tumor, Endometrioid carcinoma, Endometriosis, Endophthalmitis, Endotoxic shock, Entamoeba histolytica infection, Enteritis, Enterobacter cloacae infection, Enterobacter infection, Enterobacteriaceae infection, Enterococcus faecalis infection, Enterococcus faecium infection, Enterococcus infection, Enterocolitis, Enterocytozoonidae infection, Enterovirus 71 infection, Enterovirus infection, Enuresis, Enzyme deficiency, Eosinophilia, Eosinophilic esophagitis, Eosinophilic granuloma, Ependymoma, Epicondylitis, Epidermolysis bullosa, Epidermolysis bullosa dystrophica, Epididymitis, Epididymo-orchitis, Epilepsy, Epistaxis, Epstein Barr virus infection, Erectile dysfunction, Erysipelas, Erythema, Erythematosquamous skin disease, Erythrocyte disorder, Erythropoietic protoporphyria, Escherichia coli infection, Esophageal disease, Esophageal function disorder, Esophageal hemorrhage, Esophageal varices, Esophagitis, Esophagus tumor, Essential fatty acid deficiency, Essential hypertension, Essential tremor, Estrogen deficiency, Ethmoiditis, Euthryroid sick syndrome, Ewing sarcoma, Exercise induced asthma, Exocrine pancreatic insufficiency, Exophthalmos, Extrapyramidal syndrome, Extrinsic allergic alveolitis, Eyelid disease, Fabry disease, Facial nerve disease, Facioscapulohumeral muscular dystrophy, Factor I deficiency, Factor II deficiency, Factor IX deficiency, Factor VII deficiency, Factor VIII deficiency, Factor XIII deficiency, Fallopian tube cancer, Fallopian tube disease, Fallopian tube tumor, Familial adenomatous polyposis, Familial amyloid neuropathy, Familial cold autoinflammatory syndrome, Familial dysautonomia, Familial hypercholesterolemia, Familial mediterranean fever, Familial thrombotic microangiopathy, Fanconi anemia, Fatigue, Fatty acid metabolism disorder, Fatty acid oxidation disorder, Fatty liver disease, Febrile neutropenia, Fecal incontinence, Felty syndrome, Female birth control, Female contraception, Female genital tract infection, Female genital tract pain, Female hypogonadism, Female infertility, Female sexual dysfunction, Fetal alcohol syndrome, Fetal growth restriction, Fever, Fibrocystic breast disease, Fibrodysplasia ossificans progressiva, Fibromuscular dyplasia, Fibromyalgia, Fibrosarcoma, Fibrosis, Filariasis, Fistula, Flatulence, Flavivirus infection, Focal segmental glomerulosclerosis, Folic acid deficiency, Follicle center lymphoma, Folliculitis, Foot and mouth disease virus infection, Foot ulcer, Fragile X syndrome, Francisella tularensis infection, Friedreich ataxia, Frontotemporal dementia, Fucosidosis, Functional bowel disorder, Fungal eye infection, Fungal infection, Fungal pneumonia, Fungal respiratory tract infection, Fungal urinary tract infection, Fusarium infection, Fusobacterium infection, Galactorrhea, Gallbladder disease, Gallbladder tumor, Gallstones, Ganglioneuroblastoma, Gastric injury, Gastric motility disorder, Gastritis, Gastroenteritis, Gastroesophageal reflux, Gastrointesinal fistula, Gastrointestinal bleeding, Gastrointestinal disease, Gastrointestinal function disorder, Gastrointestinal infection, Gastrointestinal inflammation, Gastrointestinal investigative surgical procedure, Gastrointestinal motility disorder, Gastrointestinal pain, Gastrointestinal stromal tumor, Gastrointestinal tumor, Gastrointestinal ulcer, Gastroparesis, Gastrospasm, Gaucher disease, Gauchers disease type I, Gauchers disease type II, Gauchers disease type III, General anesthesia, Generalized anxiety disorder, Genetic disorder, Genital herpes, Genital tract infection, Genital tract tumor, Genital ulcer, Genitourinary disease, Genitourinary tract tumor, Germ cell and embryonic cancer, Gestational diabetes, Giant cell bone tumor, Giardia lamblia infection, Gigantism, Gingiva disease, Gingivitis, Gitelman syndrome, Glaucoma, Glaucoma surgical procedure, Glioblastoma, Glioma, Gliosarcoma, Globoid cell leukodystrophy, Glomerular disease, Glomerulonephritis, Glucose intolerance, Glycogen storage disease, GM2 gangliosidosis, Goiter, Gonad tumor, Gout, Graft versus host disease, Gram negative bacterium infection, Gram positive bacterium infection, Granulocytopenia, Granuloma, Granulosa cell tumor, Graves disease, Group A Streptococcus infection, Growth disorder, Growth hormone deficiency, Guillain Barre syndrome, Gynecological disorder, Gynecomastia, Habitual abortion, Haemophilus ducreyi infection, Haemophilus infection, Haemophilus influenzae infection, Haemophilus parainfluenzae infection, Hairy cell leukemia, Halitosis, Hallucination, Hantavirus infection, Hashimotos disease, Head and neck tumor, Head injury, Headache, Healing, Hearing disorder, Hearing loss, Heart arrhythmia, Heart disease, Heart injury, Heart neoplasm, Heart transplant rejection, Heart transplantation, Heart valve disease, Heavy metal poisoning, Hebephrenic schizophrenia, Helicobacter infection, Helicobacter pylori infection, HELLP syndrome, Helminth infection, Hemangioma, Hemangiopericytoma, Hemangiosarcoma, Hematological disease, Hematological neoplasm, Hematoma, Hematopoietic stem cell transplantation, Hematuria, Hemiplegia, Hemochromatosis, Hemoglobinopathy, Hemolytic anemia, Hemolytic uremic syndrome, Hemophilia, Hemorrhagic enteritis, Hemorrhagic shock, Hemorrhagic stroke, Hemorrhoids, Hemosiderosis, Henoch Schonlein purpura, Heparin induced thrombocytopenia, Hepatic cyst, Hepatic edema, Hepatic encephalopathy, Hepatic insufficiency, Hepatic porphyria, Hepatitis, Hepatitis A virus infection, Hepatitis B virus infection, Hepatitis C virus infection, Hepatitis D virus infection, Hepatitis E virus infection, Hepatitis virus infection, Hepatobiliary disease, Hepatobiliary system tumor, Hepatoblastoma, Hepatocellular carcinoma, Hepatomegaly, Hepatorenal syndrome, Hepatotoxicity drug-induced, Hereditary angioedema, Hereditary hemorrhagic telangiectasia, Hereditary inclusion body myositis, Hereditary neoplastic syndrome, Hereditary spastic paraplegia, Hermansky-Pudlak syndrome, Hernia, Herpangina, Herpes simplex virus infection, Herpes virus infection, Herpesvirus infection, Herpesvirus type 8 infection, Herpetic keratitis, Heterotopic ossification, Hiccup, Hidradenitis, Hidradenitis suppurativa, High grade B-cell lymphoma, Hippel Lindau syndrome, Hirsutism, Histiocytoma, Histiocytosis, Histoplasma infection, HIV associated dementia, HIV infection, HIV-1 infection, HIV-2 infection, HIV-associated lipodystrophy, Hoarseness, Hodgkins disease, Homocystinuria, Hookworm infection, Hormone deficiency, Hormone dependent prostate cancer, Hormone refractory prostate cancer, Hot flashes, HSV-1 infection, HSV-2 infection, Human T cell leukemia virus 1 infection, Hunter syndrome, Huntingtons chorea, Hurler syndrome, Hutchinson Gilford progeria syndrome, Hydronephrosis, Hyperactivity, Hyperaldosteronism, Hyperalgesia, Hyperammonemia, Hyperandrogenism, Hyperbilirubinemia, Hypercalcemia, Hypercholesterolemia, Hyperemesis gravidarum, Hyperemia, Hypereosinophilic syndrome, Hypergastrinemia, Hyperglycemia, Hyperhidrosis, Hyperinsulinemia, Hyperkalemia, Hyperlipidemia, Hyperlipoproteinemia, Hyperlipoproteinemia type I, Hyperlipoproteinemia type IIb, Hyperlipoproteinemia type III, Hypermenorrhea, Hypernatremia, Hyperopia, Hyperoxaluria, Hyperoxia, Hyperparathyroidism, Hyperphagia, Hyperphosphatemia, Hyperpigmentation, Hyperplasia, Hyperprolactinemia, Hypersensitivity, Hypersomnia, Hypersplenism, Hypertension, Hyperthermia, Hyperthyroidism, Hypertriglyceridemia, Hypertrophic cardiomyopathy, Hypertrophic skin disease, Hypertrophy, Hyperuricemia, Hypervolemia, Hypoactive sexual desire disorder, Hypoalbuminemia, Hypoalphalipoproteinemia, Hypocalcemia, Hypochondriasis, Hypogalactia, Hypogammaglobulinemia, Hypoglycemia, Hypogonadism, Hypokalemia, Hypomagnesemia, Hypomania, Hyponatremia, Hypoparathyroidism, Hypophosphatasia, Hypophosphatemia, Hypopituitarism, Hypotension, Hypothermia, Hypothyroidism, Hypothyroxinemia, Hypotrichosis, Hypovolemia, Hypoxia, Hypozincemia, Ichthyosis, Idiopathic pulmonary arterial hypertension, Idiopathic pulmonary fibrosis, IgA nephropathy, Ileum disease, Ileus, Illegal drug overdose, Immediate type hypersensitivity, Immune deficiency, Immune disorder, Immune thrombocytopenic purpura, Immunization, Immunoglobulin G deficiency, Impetigo, Impulse control disorder, Inappropriate ADH syndrome, Inborn error of metabolism, Inclusion body myositis, Indigestion, Infant colic, Infantile spasms, Infection by anatomical location, Infectious arthritis, Infectious disease, Infectious meningitis, Infectious mononucleosis, Infertility, Inflammatory bowel disease, Inflammatory breast cancer, Inflammatory disease, Influenza virus A infection, Influenza virus B infection, Influenza virus infection, Injury, Inner ear disease, Insomnia, Insulin dependent diabetes, Insulin resistance, Intellectual impairment, Intermediate uveitis, Intermittent claudication, Intermittent explosive disorder, Interstitial cystitis, Interstitial lung disease, Intervertebral disc disease, Intestine disease, Intestine infection, Intestine injury, Intestine tumor, Intoxication, Intracranial aneurysm, Intracranial hemorrhage, Intracranial hypertension, Intracranial vasospasm, Iodine deficiency, Iris disease, Iron deficiency anemia, Iron metabolism disorder, Iron overload, Irritable bowel syndrome, Ischemia, Ischemic heart disease, Ischemic stroke, Islet cell transplant rejection, Jansky-Bielschowsky disease, Japanese encephalitis virus infection, Jaundice, Jaw disease, Job syndrome, Joint disease, Joint infection, Joint injury, Juvenile myoclonic epilepsy, Juvenile rheumatoid arthritis, Kaposis sarcoma, Kawasaki disease, Keratitis, Keratoconjunctivitis, Keratoconus, Keratosis, Kidney dialysis, Kidney transplant rejection, Kidney transplantation, Klebsiella infection, Klebsiella pneumoniae infection, Kleptomania, Klinefelter syndrome, Knee fracture, Knee injury, Kwashiorkor, Labor complication, Lacrimal gland disease, Lactation disorder, Lactic acidosis, Lactobacillales infection, Lactose intolerance, Lambert-Eaton syndrome, Lamellar ichthyosis, Large cell lung carcinoma, Laron syndrome, Laryngismus, Laryngitis, Laryngopharyngitis, Laryngotonsillitis, Larynx disease, Larynx tumor, Latent autoimmune adult diabetes, Lateral epicondylitis, Lebers hereditary optic atrophy, Left heart disease pulmonary hypertension, Leg ulcer, Legg Calve Perthes disease, Legionella pneumophila infection, Leigh disease, Leiomyosarcoma, Leishmania braziliensis infection, Leishmania donovani infection, Leishmania infection, Leishmania tropica infection, Lennox Gastaut syndrome, Lentiginosis, Lentigo senilis, Leptospiraceae infection, Lesch Nyhan syndrome, Leukemia, Leukocyte disorder, Leukoencephalopathy, Leukopenia, Leukopenia drug induced, Leukoplakia, Lewy body dementia, Lice infestation, Lichen, Limb development disorder, Limb girdle muscular dystrophy, Lipid metabolism disorder, Lipid storage diseases, Lipodystrophy, Lipoma, Lipoprotein deficiency, Lipoprotein metabolism disorder, Liposarcoma, Liver abscess, Liver cirrhosis, Liver disease, Liver failure, Liver fibrosis, Liver infection, Liver injury, Liver transplant rejection, Liver transplantation, Liver tumor, Long QT syndrome, Louse infestation, Lower back pain, Lower limb fracture, Lower respiratory tract infection, Lung abscess, Lung disease, Lung disease pulmonary hypertension, Lung embolism, Lung infection, Lung inflammation, Lung injury, Lung malformation, Lung transplant rejection, Lung transplantation, Lung tumor, Lupus nephritis, Lyme disease, Lymphadenitis, Lymphadenopathy, Lymphangioleiomyomatosis, Lymphangiosarcoma, Lymphangitis, Lymphatic system disease, Lymphedema, Lymphoblastic leukemia, Lymphocele, Lymphocytopenia, Lymphocytosis, Lymphoid leukemia, Lymphoma, Lymphoplasmacytoid lymphoma, Lymphoproliferative disease, Lysosomal acid lipase deficiency, Machado-Joseph disease, Macroadenoma, Macroglobulinemia, Macular degeneration, Macular disease, Macular edema, Major depressive disorder, Malabsorption syndrome, Malassezia infection, Male contraception, Male genital system disease, Male hypogonadism, Male infertility, Male osteoporosis, Male sexual dysfunction, Malignant hyperthermia, Malnutrition, Mandible disease, Mania, Mantle cell lymphoma, Marfan syndrome, Marginal zone B-cell lymphoma, Maroteaux-Lamy syndrome, Mastalgia, Mastitis, Mastocytosis, Mastoiditis, Measles virus infection, Meconium aspiration syndrome, Medial epicondylitis, Medullary thyroid cancer, Medulloblastoma, Melancholia, Melanoma, MELAS syndrome, Melasma, Melioidosis, Membranous glomerulonephritis, Meniere disease, Meningeal neoplasm, Meningioma, Meningitis, Menopause, Menorrhagia, Menstruation disorder, Merkel cell carcinoma, Mesothelioma, Metabolic bone disease, Metabolic brain disease, Metabolic disorder, Metabolic syndrome X, Metachromatic leukodystrophy, Metal intoxication, Metastasis, Metastatic bladder cancer, Metastatic brain cancer, Metastatic breast cancer, Metastatic CNS cancer, Metastatic colon cancer, Metastatic colorectal cancer, Metastatic esophageal cancer, Metastatic gastrointestinal cancer, Metastatic head and neck cancer, Metastatic hepatobiliary cancer, Metastatic liver cancer, Metastatic lung cancer, Metastatic non small cell lung cancer, Metastatic ovary cancer, Metastatic pancreas cancer, Metastatic prostate cancer, Metastatic rectal cancer, Metastatic renal cancer, Metastatic stomach cancer, Metastatic testicular cancer, Metastatic urinary tract cancer, Methylmalonic acidemia, Metrorrhagia, Microalbuminuria, Microangiopathy, Microsporidial infection, Middle ear disease, Migraine, Migraine with aura, Migraine without aura, Mild cognitive impairment, Milk hypersensitivity, Miosis, Mite infection, Mitochondrial disease, Mitochondrial myopathy, Mitochondrial optic neuropathy, Mitral valve disease, Mitral valve insufficiency, Mitral valve stenosis, Mixed urinary incontinence, Molluscum contagiosum infection, Mood disorder, Moraxella catarrhalis infection, Morganella infection, Morning sickness, Morquio syndrome, Motion sickness, Motor neurone disease, Mouth disease, Mouth inflammation, Mouth tumor, Mouth ulcer, Movement disorder, MRSA infection, Muckle Wells syndrome, Mucopolysaccharidosis type I, Mucor infection, Mucositis, Multidrug resistant infection, Multi-infarct dementia, Multiple endocrine neoplasia type 1, Multiple endocrine neoplasia type 2a, Multiple hamartoma syndrome, Multiple myeloma, Multiple organ failure, Multiple sclerosis, Multiple system atrophy, Mumps virus infection, Muscle disease, Muscle hypertonia, Muscle injury, Muscle spasm, Muscle wasting disease, Muscle weakness, Muscular dystrophy, Musculoskeletal disease, Musculoskeletal pain, Myalgia, Myasthenia gravis, Mycobacterium avium infection, Mycobacterium infection, Mycobacterium leprae infection, Mycobacterium tuberculosis infection, Mycoplasma infection, Mycoplasma pneumoniae infection, Mycosis fungoides, Mydriasis, Myelitis, Myelodysplastic syndrome, Myelofibrosis, Myeloid leukemia, Myeloproliferative disorder, Myocardial disease, Myocardial infarction, Myocarditis, Myoclonic seizure, Myoclonus, Myoma, Myopathy, Myopia, Myositis, Myotonic dystrophy, Myotonic dystrophy type 1, N-acetylglutamate synthase deficiency, Nail disease, Narcolepsy, Narcotic dependence, Nasal congestion, Nasal polyps, Nasopharyngeal carcinoma, Nasopharynx tumor, Nausea, Nausea drug-induced, Necrosis, Necrotizing acute pancreatitis, Necrotizing enterocolitis, Neisseria gonorrhoeae infection, Neisseria infection, Neisseria meningitidis infection, Neisseria meningitidis meningitis, Nelson syndrome, Nematode infection, Neonatal respiratory distress syndrome, Neoplasm, Neoplasm by site, Neoplastic meningitis, Nephritis, Nephroblastoma, Nephrocalcinosis, Nephrogenic diabetes insipidus, Nephrogenic fibrosing dermopathy, Nephropathic cystinosis, Nephrosclerosis, Nephrotic syndrome, Nephrotoxicity, Nerve injury, Nerve tumor, Nervous system development disorder, Nervous system inflammation, Neuritis, Neurobehavioral disorder, Neuroblastoma, Neurodegenerative disease, Neuroendocrine tumor, Neurofibromatosis, Neurofibromatosis type I, Neurofibromatosis type II, Neurogenic bladder, Neurological disease, Neuroma, Neuromuscular blockade, Neuromuscular disease, Neuromyelitis optica, Neuropathic pain, Neuropathy, Neurosyphilis, Neurotoxicity drug-induced, Neutropenia, Nevus, Nicotine dependence, Niemann Pick disease, Niemann Pick disease type B, Niemann Pick disease type C, Niemann Pick disease type 01, Niemann Pick disease type C2, Nocturia, Nodular goiter, Noise induced hearing loss, Non alcoholic fatty liver disease, Non segmental vitiligo, Non traumatic brain injury, Non-alcoholic steatohepatitis, Non-Hodgkin lymphoma, Non-Hodgkin lymphoma, B-cell acute lymphoblastic leukemia, Non-insulin dependent diabetes, Non-proliferative diabetic retinopathy, Non-small-cell lung cancer, Noonan syndrome, Norovirus infection, Norwalk virus infection, Nose disease, Nutritional deficiency, Nutritional disorder, Nyctalopia, Nystagmus, Obesity, Obsessive compulsive disorder, Obsessive compulsive disorder; Obstructive intestinal disease, Obstructive sleep apnea, Occlusive coronary artery disease, Ocular disease, Ocular hypertension, Ocular infection, Ocular inflammation, Ocular injury, Ocular melanoma, Ocular neovascular disorder, Ocular pain, Ocular surgical procedure, Ocular tumor, Olfaction disorder, Oligodendroglioma, Oligospermia, Oliguria, Onchocerciasis, Onychomycosis, Open angle glaucoma, Opiate dependence, Opsoclonus myoclonus syndrome, Optic nerve disorder, Optic neuritis, Oral mucositis, Orbital inflammatory disease, Organ transplantation, Ornithine transcarbamylase deficiency, Orthomyxovirus infection, Orthostatic hypotension, Ostealgia, Osteitis, Osteoarthritis, Osteoblastoma, Osteochondritis, Osteodystrophy, Osteogenesis imperfecta, Osteolysis, Osteomyelitis, Osteonecrosis, Osteopenia, Osteopetrosis, Osteoporosis, Osteosarcoma, Otitis, Otitis externa, Otitis media, Otorhinolaryngological disease, Otorhinolaryngological infection, Otosclerosis, Ovarian clear cell carcinoma, Ovarian hyperstimulation syndrome, Ovary cyst, Ovary disease, Ovary tumor, Overactive bladder, Pagets bone disease, Pain, Palpitation, Pancoast syndrome, Pancreas disease, Pancreas islet disease, Pancreas transplant rejection, Pancreas transplantation, Pancreas tumor, Pancreatic cystadenocarcinoma, Pancreatic ductal adenocarcinoma, Pancreatic endocrine tumor, Pancreatic intraductal papillary-mucinous tumor, Pancreatitis, Pancytopenia, Panic disorder, Papillary thyroid tumor, Papillomavirus infection, Papular skin disease, Paracetamol overdose, Paracoccidioidomycosis, Paraganglioma, Parainfluenza virus infection, Paralysis, Paranasal sinus disease, Paranoid schizophrenia, Paraproteinemia, Parapsoriasis, Parasitic infection, Parasomnia, Parathyroid disease, Parathyroid tumor, Paratuberculosis, Paresthesia, Parkinsonism, Parkinsons disease, Parkinsons disease dementia, Paronychia, Paroxysmal nocturnal hemoglobinuria, Paroxysmal supraventricular tachycardia, Paroxysmal tachycardia, Paroxysmal ventricular tachycardia, Partial seizure, Parturition, Parvovirus infection, Patent ductus arteriosus, Peanut hypersensitivity, Pediatric psychiatric disorder, Pediatric varicella zoster virus infection, Pediculosis capitis infestation, Pedophilia, Pelvic inflammatory disease, Pemphigoid, Pemphigus, Penile induration, Penis disease, Penis tumor, Peptic ulcer, Peptostreptococcus infection, Perennial allergic rhinitis, Perianal fistula, Periarthritis, Periarticular joint disease, Pericardial disease, Pericarditis, Periodontal disease, Periodontitis, Peripheral arterial occlusive disease, Peripheral nervous system disease, Peripheral neuropathy, Peripheral T-cell lymphoma, Peripheral vascular disease, Peritoneal disease, Peritoneal tumor, Peritonitis, Peritonsillitis, Periventricular leukomalacia, Peroxisome disease, Persistent pulmonary hypertension of the newborn, Personality disorder, Pervasive child developmental disorder, Peutz Jegher syndrome, Phantom limb, Pharyngitis, Pharynx tumor, Phenylketonuria, Pheochromocytoma, Phimosis, Phlebitis, Phlebothrombosis, Phobia, Picornavirus infection, Pigment disorder, Pituitary adenoma, Pituitary disease, Pituitary tumor, Pityriasis, Pityriasis capitis infection, Placenta disease, Placenta retention, Planned abortion, Plasma cell dyscrasia, Plasmacytoma, Plasmodium falciparum infection, Plasmodium infection, Plasmodium knowlesi infection, Plasmodium malariae infection, Plasmodium vivax infection, Platyhelminth infection, Pleural disease, Pleurisy, Pleuropneumonia, Pneumocystis carinii infection, Pneumonia, POEMS syndrome, Poison intoxication, Poliovirus infection, Polyarteritis nodosa, Polychondritis, Polycystic kidney disease, Polycystic liver disease, Polycystic ovary syndrome, Polycythemia, Polycythemia vera, Polymorphus light eruption, Polymyalgia rheumatica, Polymyositis, Polyneuropathy, Polyomavirus infection, Polyostotic fibrous dysplasia, Polyp, Polypoidal choroidal vasculopathy, Polyuria, Pompes disease, Porphyria, Porphyria cutanea tarda, Port wine stain, Portal hypertension, Post transplant lymphoproliferative disease, Post traumatic stress disorder, Postherpetic neuralgia, Postmenopausal osteoporosis, Postnatal depression, Pouchitis, Prader-Willi syndrome, Precocious puberty, Precursor T-lymphoblastic lymphoma-leukemia, Pre-eclampsia, Pregnancy disorder, Premature ejaculation, Premature labor, Premature ovarian failure, Premedication, Premenstrual syndrome, Presbycusis, Presbyopia, Prevotella infection, Priapism, Primary biliary cirrhosis, Primary hyperparathyroidism, Primary mediastinal large B-cell lymphoma, Primary sclerosing cholangitis, Prion infection, Proctitis, Progestogen deficiency, Progressive multifocal leukoencephalopathy, Progressive supranuclear palsy, Prolactinoma, Proliferative diabetic retinopathy, Prolymphocytic leukemia, Prophylaxis, Propionibacterium acnes infection, Propionic acidemia, Prostate disease, Prostate hyperplasia, Prostate tumor, Prostatectomy, Prostatic intraepithelial neoplasia, Prostatitis, Protein C deficiency, Protein energy deficiency, Proteinosis, Proteinuria, Proteus infection, Proteus mirabilis infection, Protozoal infection, Providencia infection, Prurigo, Prurigo nodularis, Pruritus, Pseudohypoaldosteronism, Pseudomembranous colitis, Pseudomonas aeruginosa infection, Pseudomonas infection, Pseudoobstruction, Pseudotumor cerebri, Pseudoxanthoma elasticum, Psoriasis, Psoriatic arthritis, Psychiatric disorder, Psychomotor disorder, Psychosexual disorder, Psychotic disorder, Pterygium, Pulmonary artery hypertension, Pulmonary edema, Pulmonary eosinophilia, Pulmonary fibrosis, Pulmonary heart disease, Pulmonary hypertension, Pulmonary valve insufficiency, Pulmonary valve stenosis, Pulpitis, Pupil disorder, Purpura, Pyelonephritis, Pyoderma gangrenosum, Pyromania, Pyruvate carboxylase deficiency, Pyuria, Q fever, Quadriplegia, Rabies virus infection, Radiation sickness, Radiotherapy induced emesis, Raynauds disease, Rectal disease, Rectal tumor, Rectosigmoiditis, Refractive error, Reiter syndrome, Renal artery obstruction, Renal cell carcinoma, Renal colic, Renal disease, Renal failure, Renal injury, Renal insufficiency, Renal osteodystrophy, Renal pain, Renal sarcoma, Renal tumor, Renovascular hypertension, Reperfusion injury, Reproductive disorder, Resistant hypertension, Respiratory congestion, Respiratory depression, Respiratory disease, Respiratory disorder, Respiratory distress syndrome, Respiratory failure, Respiratory insufficiency, Respiratory syncytial virus infection, Respiratory tract allergy, Respiratory tract bleeding, Respiratory tract infection, Respiratory tract inflammation, Respiratory tract tumor, Restenosis, Restless legs syndrome, Retinal artery occlusion, Retinal venous occlusion, Retinitis pigmentosa, Retinoblastoma, Retinopathy, Retinopathy of prematurity, Rett syndrome, Rhabdomyolysis, Rhabdomyosarcoma, Rheumatoid arthritis, Rhinitis, Rhinopharyngitis, Rhinorrhea, Rhinovirus infection, Rickets, Rigor, Rosacea, Ross River virus infection, Rotavirus infection, Rubella virus infection, Rubinstein-Taybi syndrome, Saccharomyces infection, Salivary gland disease, Salmonella infection, Salmonella typhi infection, Salmonella typhimurium infection, Sandhoff disease, Sanfilippo syndrome, Santavuori-Haltia disease, Sarcoidosis, Sarcoma, Sarcopenia, SARS coronavirus infection, Scabies infection, Scar tissue, Scedosporium infection, Schistosomiasis, Schizoaffective disorder, Schizophrenia, Schizophreniform disorder, Schizophyllum infection, Sciatica, Scleral disease, Scleritis, Scleroderma, Scoliosis, Seasonal affective disorder, Seasonal allergic rhinitis, Sebaceous gland disease, Seborrheic dermatitis, Seborrheic keratosis, Secondary hyperparathyroidism, Secondary pulmonary arterial hypertension, Seizure disorder, Seminoma, Sensorineural hearing loss, Sepsis, Septic shock, Serratia infection, Serratia marcescens infection, Sertoli-Leydig cell tumor, Severe combined immunodeficiency syndrome, Sexual compulsive disorder, Sexual dysfunction, Sezary syndrome, Shigella boydii infection, Shigella dysenteriae infection, Shigella flexneri infection, Shigella infection, Shigella sonnei infection, Shock, Short bowel syndrome, Shwachman syndrome, Shy Drager syndrome, Sialorrhea, Sickle beta 0 thalassemia, Sickle beta zero thalassemia, Sickle cell anemia, Sickle cell crisis, Sigmoiditis, Sinusitis, Sitosterolemia, Sjoegrens syndrome, Skin allergy, Skin burns, Skin cancer, Skin infection, Skin injury, Skin photosensitivity disorder, Skin rash, Skin transplantation, Skin tumor, Skin ulcer, Sleep apnea, Sleep arousal disorder, Sleep disorder, Sleep maintenance insomnia, Sly syndrome, Small intestine disease, Small-cell lung cancer, Smith Lemli Opitz syndrome, Snoring, Social phobia, Soft tissue sarcoma, Solar urticaria, Solid tumor, Somatostatinoma, Spielmeyer-Vogt disease, Spina bifida, Spinal and bulbar muscular atrophy, Spinal cord compression, Spinal cord disease, Spinal cord injury, Spinal cord tumor, Spinal cord vascular disease, Spinal disease, Spinal injury, Spinal muscular atrophy, Spinal stenosis, Spinocerebellar ataxia, Spinocerebellar degenerations, Splenic marginal zone lymphoma, Splenomegaly, Spondylarthritis, Spondylolisthesis, Spondylosyndesis, Spontaneous abortion, Sporadic inclusion body myositis, Squamous cell carcinoma, Stage II melanoma, Stage III melanoma, Stage IV melanoma, Staphylococcus aureus infection, Staphylococcus epidermidis infection, Staphylococcus infection, Staphylococcus saprophyticus infection, Stargardt disease, Status asthmaticus, Status epilepticus, Steatohepatitis, Steatorrhea, Stem cell transplantation, Stevens Johnson syndrome, Stiff person syndrome, Stomach disease, Stomach infection, Stomach tumor, Stomach ulcer, Stomatitis, Storage disease, Strabismus, Streptococcus agalactiae infection, Streptococcus group B infection, Streptococcus infection, Streptococcus mutans infection, Streptococcus pneumoniae infection, Streptococcus pyogenes infection, Stress urinary incontinence, Stroke, Sturge-Weber syndrome, Stutter, Sudden cardiac death, Suicidal ideation, Suicide, Sunburn, Supraventricular tachycardia, Surgical procedure, Sydenham chorea, Syncope, Syndrome X, Synovitis, Systemic inflammatory response syndrome, Systemic lupus erythematosus, Systemic mastocytosis, Systolic dysfunction, Systolic heart failure, Tachycardia, Tachypnea, Taenia infection, Takayasus arteritis, Tardive dyskinesia, Taste disorder, Tay Sachs disease, T-cell acute lymphoblastic leukemia, T-cell large granular lymphocyte leukemia, T-cell lymphoma, Telangiectasis, Temperature disorder, Temporal arteritis, Temporomandibular joint syndrome, Tendinitis, Tendon disease, Tendon injury, Tenosynovitis, Teratoma, Testis tumor, Testosterone deficiency, Tetralogy of Fallot, Thalassemia, Thalassemia intermedia, Thalassemia major, Thoracic aortic aneurysm, Thoracic outlet syndrome, Throat disease, Thrombasthenia, Thromboangiitis obliterans, Thrombocyte disorder, Thrombocythemia, Thrombocytopenia, Thrombocytopenic purpura, Thrombocytosis, Thromboembolism, Thrombophlebitis, Thrombosis, Thrombotic microangiopathy, Thymoma, Thymus neoplasm, Thyroid associated ophthalmopathy, Thyroid disease, Thyroid tumor, Thyroiditis, Thyrotoxicosis, Tic disorder, Tick borne encephalitis virus infection, Tinea, Tinea capitis, Tinea corporis, Tinea cruris, Tinea pedis, Tinnitus, Tissue adhesions, Tongue disease, Tonic clonic epilepsy, Tonsillitis, Tooth disease, Tooth injury, Tooth loss, Topical anesthesia, Torticollis, Tourette syndrome, Toxic epidermal necrolysis, Toxicity, Toxoplasma gondii infection, Toxoplasma infection, Tracheobronchitis, Trachoma, Transient ischemic attack, Transitional cell carcinoma, Transplant rejection, Transplantation, Transsexualism, Traumatic brain injury, Trematode infection, Tremor, Treponema infection, Treponema pallidum infection, Trichocomaceae infection, Trichomonas infection, Trichotillomania, Tricuspid atresia, Tricuspid insufficiency, Trigeminal nerve disorder, Trigeminal neuralgia, Trismus, Tropical spastic paraparesis, Trypanosoma brucei infection, Trypanosoma cruzi infection, Trypanosomiasis, Tuberous sclerosis, Tumers syndrome, Tumor lysis syndrome, Turners syndrome, Type I complex regional pain syndrome, Type I tyrosinemia, Tyrosinemia, Ulcer, Ulcerative colitis, Ulcerative proctitis, Umbilical cord stem cell transplantation, Unconsciousness, Undifferentiated schizophrenia, Unstable angina, Upper limb fracture, Upper respiratory tract infection, Ureaplasma infection, Uremia, Ureteral colic, Ureteral disease, Urethral cancer, Urethral disease, Urethritis, Urge urinary incontinence, Urinary dysfunction, Urinary incontinence, Urinary retention, Urinary tract disease, Urinary tract infection, Urinary tract tumor, Urogenital tract infection, Urolithiasis, Urticaria, Uterine cervix tumor, Uterine fibroids, Uterine hemorrhage, Uterus disease, Uterus infection, Uterus tumor, Uveal melanoma, Uveitis, Vaccination, Vagina disease, Vaginal cancer, Vaginal infection, Vaginal intraepithelial neoplasia, Vaginitis, Variant angina pectoris, Varicella zoster virus infection, Varices, Varicocele, Varicosis, Varicosis ulcer, Variola virus infection, Vascular dementia, Vascular disease, Vascular disease by process, Vascular eye disease, Vascular fistula, Vascular injury, Vascular neoplasm, Vascular occlusive disease, Vasculitis, Vasospasm, Vein disease, Vein graft failure, Venous insufficiency, Venous occlusive disease, Ventricular arrhythmia, Ventricular fibrillation, Ventricular tachycardia, Verruca vulgaris, Vertebral fracture, Vertigo, Vesicoureteral reflux, Vestibulocochlear nerve disorder, Vibrio cholerae infection, Vibrio infection, Viral encephalitis, Viral hemorrhagic fever, Viral infection, Viral pneumonia, Viral respiratory tract infection, Viridans group Streptococcus infection, Vision disorder, Vitamin B12 deficiency, Vitamin B6 deficiency, Vitamin C deficiency, Vitamin D deficiency, Vitamin deficiency, Vitamin E deficiency, Vitamin K deficiency, Vitelliform macular dystrophy, Vitiligo, Vitreous hemorrhage, Vitreous humor disease, Von Willebrands disease, VRSA infection, Vulva disease, Vulvar intraepithelial neoplasia, Vulvovaginitis, Wegener granulomatosis, Weight gain, Weight loss, West Nile virus infection, Wet age related macular degeneration, Wheat hypersensitivity, Williams-Beuren syndrome, Wilson disease, Wiskott-Aldrich syndrome, Worm infection, Wound healing, X linked dominant hypophosphatemic rickets, Xerophthalmia, Xerosis, Xerostomia, Yellow fever virus infection, Yersinia pestis infection, Zollinger-Ellison syndrome, and Zygomycete infection.

### Mapping of Genomic Signaling Centers to Elucidate Gene Signaling Networks

The present invention contemplates the mapping of GSN of each gene in the nucleus and those signaling events as they are connected to other genes through GSCs of INs.

The map may be used to provide a context for multiple INs, and to identify the signaling pathways that may be altered and consequently the creation of a GSN for every gene. Preparing a map including rules and factors may also be used as drug development platform. For example, genetic variants associated with body mass index (BMI), fasting traits, and insulin resistance may be used to in combination with the map to identify genes contributing to liver disease. Modulation of expression of these genes via perturbations of corresponding GSCs can be advantageous in liver disease therapies.

In certain embodiments, hepatocytes are used to map a healthy cell population. The liver is a source of many known genetic disease and provides homogeneous primary tissue with high availability. Additionally, most toxicology is done in the liver, which provides drug toxicity insights. In some embodiments, different stimuli, such as those described herein, are contacted with a cell to induce various GSC responses in the hepatocytes. For example, small molecules may be administered to identify liver disease genes whose expression is modulated by the small molecules. These responses provide an understanding of GSCs in liver cells: genomic positions, protein composition, and genome architecture. Alternatively, blood or muscle tissue may be mapped as a healthy population, as a disease model, or as primary disease cells. About 20 million cells are generally needed for accurate ChIP (chromatin immunoprecipitation) analysis. Therefore, in some embodiments, erythroid cells may be used to develop a conditionally immortalized cell system to produce a sufficient biomass for ChIP analysis of a cell with a disease phenotype.

In fact, a gene signaling network map or atlas may be made for any cell type in the human body.

In some embodiments, ChIP-seq and ATAC-seq (Assay for Transposase-Accessible Chromatin using sequencing) identifies genomic positions and protein compositions within GSCs to identify ODSC. In certain embodiments, the protein composition of the GSCs may also be identified using ChIP-MS. The relationships among the proteins and associated ChIP-seq and ATAC-seq targets may be used to characterize the GSCs for mapping.

In certain embodiments, cohesin ChIA-PET (Chromatin Interaction Analysis by Paired-End Tag Sequencing) may be used to decipher genome architecture by identifying cohesin-associated loops through interacting regions of chromatin. Cohesin is found at the boundaries of both CTCF-CTCF loops that form insulated neighborhoods and gene loops that include GSCs.

In some embodiments, the three-dimensional (3D) architecture is analyzed using ChIA-PET data for structural protein SMC1 (structural maintenance of chromosomes protein 1) in primary human hepatocytes. The SMC complex is associated with enhancer-promoter interactions and CTCF binding sites, as described in Ji et al., 2016, Cell Stem Cell, 18, 262-275, which is hereby incorporated by reference in its entirety. In some embodiments, computational models may be used to provide the foundation for deciphering the signaling code based on the observed genome architecture and changes in composition or genomic position.

### Identifying Changes to Genomic Signaling Centers (GSCs) During Development

The protein compositions of GSCs have been observed to change during differentiation, as described in Trompouki et al., Cell 147, 577-589 (2011), which is hereby incorporated by reference in its entirety. Cell signaling pathways use activated transcription factors associated with specific GSCs to form ODSCs, which use additional transcription factors and chromatin remodeling complexes to change the chromatin architecture and change the accessibility to certain loci. Therefore, disrupting GSCs that regulate expression of genes associated with a progenitor phenotype, may result in forming other GSCs that regulate expression of genes associated with a differentiated phenotype. In certain embodiments, the changes in the binding profile of GSCs during differentiation of erythroid cells is mapped through the process of erythropoiesis. In certain embodiments, the changes in the binding profile of an ODSC are mapped through differentiation.

Identifying Changes in a GSN from Perturbation of a Genomic Signaling Center

In certain embodiments, changes in a GSC and regulatory responses are mapped in a cell with a disease phenotype. For example, the disease phenotype is DBA in erythroid cells. In certain embodiments, the disease phenotype is present in a conditionally immortalized cell system. For example, the disease phenotype is modeled in healthy cells using a shRNA to knockdown at least one protein associated with the disease.

### Modeling of Disease

The present invention may be used to model a disease. Once one or more GSNs have been elucidated or IN and GSNs defined, such gene expression patterns can be compared to the expression profiles of cells or tissues from the disease. Any overlap would allow further insights and interrogation of GSNs not known to be associated with such disease. These results could then be used to drive hypothesis testing in other aspects of the disease population, cells, or tissues.

### Producing Gene Regulatory Mans

Embodiments herein provide a functionalization of the GSCs to treat disease or to create a platform for drug discovery based on genomic bioinformatics. These embodiments may be used to predict how to influence genetic expression with selective signals and molecules to affect diseases associated with genetic mutations. Existing drugs, reformulated drugs, and new compositions of matter are analyzed to identify perturbation stimuli with selective disease impact. For example, small molecules selected from those described herein may be administered. New drugs may be developed for rare diseases by influencing mechanisms of genetic expression. For example, gene modulation for disease prevention and mitigation through the “transmission code” of a GSC.

A regulatory map would allow the following to be readily assessable from the identification of a single gene: the other genes in that IN, the binding profile of different GSCs that turn those genes on or off, the context of different GSCs, and the signaling pathways that affect the GSC. For a drug that modulates GSCs, the genes and neighborhoods where the drug acts could also be identified, including all of the modulators that synergize or interfere with the GSC. For example, the maps may be used to confirm that a drug changes the binding profile of an ODSC.

### Deciphering the Signaling Code

In certain embodiments, the relationships among genomic position/protein compositions of the GSCs and genome architecture of cells and transcriptional responses of disease genes and how these are affected by perturbation stimuli targeting the GSCs are analyzed. For example, disease genes whose expression is modulated in the liver are identified. In certain embodiments, relationships are analyzed after targeting an ODSC with a small molecule.

### Conducting Targeted Small Molecule Screen

A small molecule screen may be performed to identify small molecules that act through GSCs of an insulated neighborhood to after gene signaling networks which may modulate expression of a select group of disease genes, for example, by administering known signaling agonists/antagonists. Databases are compiled of target disease genes whose insufficient expression contributes to a disease phenotype of interest. Credible hits are identified and validated by the small molecules that are known to work through a GSC and modulate expression of target disease genes.

Among targets whose reduced expression or haplosufficiency contributes to a disease phenotype, additional genes that experience a change in expression are identified. Orthogonal gene expression assay may be used on cell from multiple donors to validate the targets. These results may be used to derive a set of rules, also referred to as a “signaling code” or a “transmission code”, that permits changes in gene expression due to small molecule compounds.

### Personalized Medicine

In certain embodiments, the platform described here may be used to define molecules that act through GSCs of an IN to after GSNs associated with a risk allele or any genomic mutation. For example, the platform may be used of define molecules that act through an ODSC. In one aspect, the risk allele is in the liver. Identification of risk alleles in an individual may allow a determination of the safe range for them to occur. Further, cohort based dose escalation based on the prevalence of a risk allele in a population may allow for a design of individual dose escalation once risk alleles are identified in the individual.

### Determining Drug Efficacy

In certain embodiments, identifying the relationship between changes in GSCs of an IN to alter GSNs and risk alleles present in an individual may be used to determine drug efficacy prior to administration of the drug. In certain embodiments, at least one of the GSCs is an ODSC. For example, the “transmission code” of the GSCs is used to simulate the effects of the drug in the individual.

### Patient Stratification

In certain embodiments of the methods described herein, patients are stratified or selected for treatment with a stimulus based on screening cells of the patient for response to the stimulus to measure differential gene expression.

### Drug Discovery Platform

A drug screen may be performed by using a drug that is already known to modulate a specific pathway or GSC. Expression of disease-associated genes is measured to prepare a map of factors that regulate each gene.

### II. Bioinformatics Platform

In certain embodiments, a bioinformatics platform is developed from the GSN maps and “transmission code” described above. The bioinformatics platform provides a process for developing a therapeutic strategy for an individual or a disease. For example, the platform provides automated discovery of treatment for an individual with a particular set of risk alleles.

### Databases

In certain embodiments, the bioinformatics platform includes databases of compounds and genetic diseases. For example, the compound database includes the name of the compound, the target protein/gene, and affected signaling pathways. The compounds may be identified from at least one of the following databases: Drugbank, PharmGKB, MedChemExpress, and Selleckchem. Alternatively, the compound database may include the stimuli described herein.

The database may include the causal gene mutation, disease prevalence, syndrome, and pathogenesis. For example, the genetics disease database contains diseases associated with the liver and/or blood. The gene associated with a disease may be selected from one of the following databases: CTD, disease ontology, and disease signature genes. The signaling pathways and functional elements may be selected from the following next generation sequencing databases: ENCODE and GTEx. Comprehensive compound information may be used to identify compounds, annotate disease association genes through published NGS data. Identifying patterns Will allow for predicting how a compound will behave in a subject.

In some embodiments, a database system of all GSNs is contemplated. Such database system may comprise hardware and software, a user interface and database storage. The database may be connected via any means to the cloud or stored for cloud computing. Such connectivities may be wireless and may be implemented on standard computer hardware such as desktops or laptops but may also be implemented on PDA (personal display devices) such as cell phones and the like.

### Locating Occupancy-Dependent Signaling Centers

In certain embodiments, locations ODSCs are characterized by region of the genome bound by(i) at least 2 signaling proteins and comprises: (ii) a H3K27 chemical modification, or independently at least one of a bromodomain-containing protein (Brd), a transcriptional coactivator, and at least two master transcription factors bound to the region. The transcriptional coactivator is histone acetyltransferase p300. In additional embodiments, the chemical modification is H3K27ac.

In certain embodiments, ChIP-seq is used to identify the presence of the characteristics of an ODSC. Alternatively, ATAC-seq is used to identify the presence of the characteristics of an ODSC. Examples of locations of ODSCs include the locations in Tables 43-67. In certain embodiments, the nucleic acid sequence for the region of the genome is selected from one of SEQ ID NO: 32,627-71,281.

### Prioritization of Genome Interactions

In certain embodiments of the platform described herein, machine learning is used to provide in silico prioritization of genome interactions, estimates of confidence for an interaction, and an iterative/systemic framework for identifying previously unidentified interactions. Determinations established through machine learning have been previously observed to be translatable to other areas. In certain embodiments of the platform described herein, machine learning is used to develop an automated system to identify patterns too complex for naïve observation and answer questions that cannot be tested experimentally.

In certain embodiments, machine learning may be used to predict how signaling pathways control gene expression levels. For example, a model may be designed to correlate signaling pathways with gene expression based on existing sample datasets. The model is then trained iteratively using sets of training data and validation data. The trained model is used for predicting pathway to gene expression correlations, for example, identifying which pathway controls a targeted set of genes. In certain embodiments, machine learning may also be used to predict how compounds impact signaling pathways.

In certain embodiments, machine learning may be used to predict similarity of a compound to a profiled library, such as the databases of compounds described herein. For example, features of the new compound may be projected onto a latent space and clustered against the features of the compounds from the profiled library, and the distance between the features is scored. The feature projection process is iterated based on the scoring result until optimal fitness is reached, and then similary is determined.

In certain embodiments, machine learning may also be used in drug discovery. For example, machine learning may be used to cluster expression data from diseased tissue or drug treatments. In some embodiments, machine learning may be used for patient stratification by segregating a population by response to stimuli or by studying underlying patterns. In some embodiments, machine learning may be used to guide target selection by predicting the genetic sequence variation on expression/phenotype. In certain embodiments, machine learning may be used to determine whether a compound will cause expression change at a particular gene by predicting causal pathway/gene regulation association and predicting a dynamic range of expression change under control of pathways associated with the compound's effects. In certain embodiments, machine learning may be used to determine the direct and indirect effects of compound treatment by predicting a regulatory cascade of pathway perturbation. In certain embodiments, machine learning may be used to identify key assays to perform when mapping new cell types by calculating prediction gain from variable feature selection. In certain embodiments, machine learning may be used to identify treatments that will translate into different organisms by predicting treatment on other model organisms. In certain embodiments, machine learning may be used to predict which pathway controls a targeted set of disease genes.

### III. Formulations and Delivery

### Pharmaceutical Compositions

According to the present invention the compositions may be prepared as pharmaceutical compositions. It Will be understood that such compositions necessarily comprise one or more active ingredients and, most often, a pharmaceutically acceptable excipient.

Relative amounts of the active ingredient, a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the pharmaceutical compositions described herein may comprise at least one payload. As a non-limiting example, the pharmaceutical compositions may contain 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients or subjects. In some embodiments, compositions are administered to human cells (e.g., hepatocytes, erythroid cells).

### Formulations

Formulations of the present invention can include, without limitation, saline, liposomes, lipid nanoparticles, polymers, peptides, proteins, cells transduced with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients.

Formulations of the compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

### Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Whams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, the pharmaceutical compositions formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present invention may be approved by the US Food and Drug Administration (FDA).

In one embodiment, the pharmaceutical compositions comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl.Alpha.-Ionone; Almond Oil; Alpha-Terpineol; Alpha-Tocopherol; Alpha-Tocopherol Acetate, D1-; Alpha-Tocopherol, DI-; Aluminum Acetate; Aluminum Chlorhydroxy Allantoinate; Aluminum Hydroxide; Aluminum Hydroxide—Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, DI-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Ruid 360; Dimethyl Isosorbide; Dimethyl Sulfoxide; Dimethylaminoethyl Methacrylate—Butyl Methacrylate—Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatilglycerol, DI-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor Fig 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O Fl-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, DI-; Glutathione; Glycerin; Glycerol Ester Of Hydrogenated Rosin; Glyceryl Citrate; Glyceryllsostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate—Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa.S); Hypromellose 2910 (15000 Mpa.S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate—Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, Dl-; Lactic Acid, L-; Lactobionic Acid; Lactose; Lactose Monohydrate; Lactose, Hydrous; Laneth; Lanolin; Lanolin Alcohol—Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa.S); Methylcelluloses; Methylchloroisothiazolinone; Methylene Blue; Methylisothiazolinone; Methylparaben; Microcrystalline Wax; Mineral Oil; Mono And Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/01eth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride):Sebacic Acid; Poly(Dimethylsiloxane/Methylvinylsiloxane/Methylhydrogensiloxane) Dimethylvinyl Or Dimethylhydroxy Or Trimethyl Endblocked; Poly(DI-Lactic-Co-Glycolic Acid), (50:50; Poly(DI-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene—Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether.Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, DI-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical composition formulations disclosed herein may include cations or anions. In one embodiment, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations of the invention may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

### Vectors

In some embodiments, perturbation stimulus of the present invention may be encoded in a vector or contained within a vector (e.g., a recombinant expression vector). Such perturbation stimulus may be any nucleic acid or proteinaceous molecule necessary to carry out the aspects of the methods described herein.

In one embodiment, the nucleic acid encoding a site-specific nuclease (e.g., a CRISPR/Cas enzyme), a guide nucleic acid, and/or a hybridizing oligonucleotide necessary to carry out the aspects of the methods of the disclosure may be encoded in or contained within in a vector.

The term “vector” refers to a vehicle, preferably a nucleic acid molecule, capable of transporting another nucleic acid to which it has been linked. One type of vector is a “plasmid,” which refers to a circular double-stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some embodiments, vectors can be capable of directing the expression of nucleic acids to which they are operatively linked. Such vectors are referred to herein as “recombinant expression vectors”, or more simply “expression vectors”, which serve equivalent functions. Expression vectors that may be used to carry out certain aspects of the present invention include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Additional vectors contemplated for eukaryotic target cells include, but are not limited to, the vectors pXT1, pSG5, pSVK3, pBPV, pMSG, and pSVLSV40 (Pharmacia). Other vectors can be used so long as they are compatible with the host cell.

### IV. Administration and Dosing

### Administration

In one aspect of the method, the pharmaceutical composition may be administered via a route such as, but not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

Modes of administration include injection, infusion, instillation, and/or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some examples, the route is intravenous. For the delivery of cells, administration by injection or infusion can be made.

The cells can be administered systemically. The phrases “systemic administration”, “administered systemically”, “peripheral administration”, and “administered peripherally” refer to the administration other than directly into a target site, tissue, or organ, such that it enters, instead, the subject's circulatory system and, thus, is subject to metabolism and other like processes.

### Dosing

The term “effective amount” refers to the amount of the active ingredient needed to prevent or alleviate at least one or more signs or symptoms of a specific disease and/or condition, and relates to a sufficient amount of a composition to provide the desired effect. The term “therapeutically effective amount” therefore refers to an amount of active ingredient or a composition comprising the active ingredient that is sufficient to promote a particular effect when administered to a typical subject. An effective amount would also include an amount sufficient to prevent or delay the development of a symptom of the disease, after the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

The pharmaceutical, diagnostic, or prophylactic compositions of the present invention may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the invention are typically formulated in unit dosage form for ease of administration and uniformity of dosage. ft will be understood, however, that the total daily usage of the compositions of the present invention may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, and route of administration; the duration of the treatment; drugs used in combination or coincidental with the active ingredient; and like factors well known in the medical arts.

In certain embodiments, pharmaceutical compositions in accordance with the present invention may be administered at dosage levels sufficient to deliver from about 0.01 mg/kg to about 100 mg/kg, from about 0.01 mg/kg to about 0.05 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

The desired dosage of the composition present invention may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

### V. Definitions

The term “biologic”, as used herein, refers to a medical product made from a variety of natural sources such as micro-organism, plant, animal, or human cells.

The term “binding profile”, as used herein, refers to the context-specific combination of biomolecules or factors identified to bind a GSC that interact to form a three-dimensional macrocomplex. Factors may include master transcription factors, signaling transcription factors, chromatin remodelers, and the like.

The term “boundary”, as used herein, refers to a point, limit, or range indicating where a feature, element, or property ends or begins. The term “insulated neighborhood boundary” or “IN boundary”, as used herein, refers to a boundary that delimits an insulated neighborhood (IN) on a chromosome. The term “primary downstream boundary”, as used herein, refers to the IN boundary located downstream of a primary neighborhood gene. The term “primary upstream boundary”, as used herein, refers to the IN boundary located upstream of a primary neighborhood gene. The term “secondary downstream boundary”, as used herein, refers to the downstream boundary of a secondary loop within a primary N. The term “secondary upstream boundary”, as used herein, refers to the upstream boundary of a secondary loop within a primary IN.

The term “borderline product”, as used herein, refers to products that do not fall within or belong to a specific category.

The term “disease-associated gene”, as used herein, refers to genes, either protein-coding or non-protein coding, including an allele whose mutation has been shown to result in the disease phenotype.

The term “downstream neighborhood gene”, as used herein, refers to a gene downstream of primary neighborhood gene that may be located within the same IN as the primary neighborhood gene.

The term “drug”, as used herein, refers to a substance other than food intended for use in the diagnosis, cure, alleviation, treatment, or prevention of disease and intended to affect the structure or any function of the body.

The term “enhancer”, as used herein, refers to regulatory DNA sequences that, when bound by transcription factors, enhance the transcription of an associated gene.

The term “gene”, as used herein, refers to a unit or segment of the genomic architecture of an organism, e.g., a chromosome. Genes may be coding or non-coding. Genes may be encoded as contiguous or non-contiguous polynucleotides. Genes may be DNA or RNA.

Historically, the term “signaling center” has been used to describe a group of cells responding to changes in the cellular environment. See, Guger et al., Developmental Biology 172: 115-125 (1995), which is incorporated by reference herein in its entirety. Similarly, the term “signaling center”, as used herein, refers to a defined region of a living organism interacting with a defined set of biomolecules, such as signaling proteins or signaling molecules (e.g., transcription factors) to regulate gene expression in a context-specific manner.

Specifically, the term “genomic signaling center” or “GSC”, i.e., a “signaling center”, as used herein, refers to regions within INs that include a context-specific combinatorial assembly of signaling molecules/signaling proteins that participate in the regulation of the genes within that IN or among more than one IN.

The term “genomic system architecture”, as used herein, refers to the organization of an individual's genome and includes chromosomes, topologically associating domains (TADs), and INs.

The term “herbal preparation”, as used herein, refers to herbal medicines that contain parts of plants, or other plant materials, or combinations as active ingredients.

The term “insulated neighborhood” or “IN”, as used herein, refers to chromosome structure formed by the looping of two interacting sites in the chromosome sequence that may comprise CCCTC-binding factor (CTCF) co-occupied by cohesin and affect the expression of genes in the IN as well as those genes in the vicinity of the IN.

The term “insulator”, as used herein, refers to regulatory elements that block the ability of an enhancer to activate a gene when located between them and contribute to specific enhancer-gene interactions.

The term “location” refers to a position of interest (i.e., base number or residue number) in a nucleic acid molecule or protein relative to the position in another reference nucleic acid molecule or protein. Corresponding positions can be determined by comparing and aligning sequences to maximize the number of matching nucleotides or residues, for example, such that identity between the sequences is greater than 90%, greater than 95%, greater than 96%, greater than 97%, greater than 98% or greater than 99%. The position of interest is then given the number assigned in the reference nucleic acid molecule. For example, if a particular polymorphism in Gene-X occurs at nucleotide 2073 of SEQ ID No. X, to identify the corresponding nucleotide in another allele or isolate, the sequences are aligned and then the position that lines up with 2073 is identified. Since various alleles may be of different length, the position designated 2073 may not be nucleotide 2073, but instead is at a position that “corresponds” to the position in the reference sequence.

The term “master transcription factor”, as used herein, refers to signaling molecules/signaling proteins which alter, whether to increase or decrease, the transcription of a target gene, e.g., a neighborhood gene and establish cell-type specific enhancers. Master transcription factors recruit additional signaling proteins, such as other transcription factors to enhancers to form GSCs.

The term “minimal insulated neighborhood” or “minimal IN”, as used herein, refers to an IN having at least one neighborhood gene and associated regulatory sequence region or regions (RSRs) which facilitate the expression or repression of the neighborhood gene such as promoter and/or enhancer and/or repressor regions, and the like.

The term “neighborhood gene”, as used herein, refers to a gene localized within an insulated neighborhood.

The term “occupancy-dependent signaling center” or “ODSC”, as used herein, refers to a RSR in the genome including binding by (i) at least 2 signaling proteins and independently (ii) at least one of a H3K27 chemical modification or binding by at least one of a bromodomain-containing protein (Brd), a transcriptional coactivator, or two or more master transcription factors. Occupancy-dependent signaling centers mediate interactions among genomic regulatory elements.

The term “penetrance”, as used herein, refers to the proportion of individuals carrying a particular variant of a gene (e.g., mutation, allele or generally a genotype, whether wild type or not) that also exhibits an associated trait (phenotype) of that variant gene and in some situations is measured as the proportion of individuals with the mutation who exhibit clinical symptoms thus existing on a continuum.

The term “primary neighborhood gene” as used herein, refers to a gene which is most commonly found within a specific IN along a chromosome.

The term “promoter” as used herein, refers to a DNA sequence that defines where transcription of a gene by RNA polymerase begins and defines the direction of transcription indicating which DNA strand will be transcribed.

The term “regulatory sequence regions” or “RSRs”, as used herein, include but are not limited to regions, sections or zones along a chromosome whereby interactions with signaling molecules/signaling proteins occur in order to after expression of a neighborhood gene.

The term “repressor”, as used herein, refers to any protein that binds to DNA and therefore regulates the expression of genes by decreasing the rate of transcription.

The terms “signaling molecule” or “signaling protein”, as used herein, refers to any entity, whether protein, nucleic acid (DNA or RNA), organic small molecule, lipid, sugar or other biomolecule, which interacts directly, or indirectly, with a RSR on a chromosome.

The term “signaling transcription factor”, as used herein, refers to signaling molecules/signaling proteins which alter, whether to increase or decrease, the transcription of a target gene, e.g., a neighborhood gene and also act as cell-cell signaling molecules/signaling proteins.

The term “small molecule”, as used herein, refers to a low molecular weight drug, i.e. <900 Daltons organic compound with a size on the order of 10⁻⁹ m that may help regulate a biological process.

The term “super-enhancers”, as used herein, refers to are large clusters of transcriptional enhancers that drive expression of genes that define cell identity.

The term “therapeutic agent”, as used herein, refers to a substance that has the ability to cure a disease or ameliorate the symptoms of the disease.

The term “therapeutic or treatment outcome”, as used herein, refers to any result or effect (whether positive, negative or null) which arises as a consequence of the perturbation of a GSC or GSN. Examples of therapeutic outcomes include, but are not limited to, improvement or amelioration of the unwanted or negative conditions associated with a disease or disorder, lessening of side effects or symptoms, cure of a disease or disorder, or any improvement associated with the perturbation of a GSC or GSN.

The term “topologically associating domains” (TADs), as used herein, refers to structures that represent a modular organization of the chromatin and have boundaries that are shared by the different cell types of an organism.

The term “transcription factors”, as used herein, refers to signaling molecules/signaling proteins which alter, whether to increase or decrease, the transcription of a target gene, e.g., a neighborhood gene.

The term “therapeutic or treatment liability”, as used herein, refers to a feature or characteristic associated with a treatment or treatment regime which is unwanted, harmful or which mitigates the therapies positive outcomes. Examples of treatment liabilities include for example toxicity, poor half-life, poor bioavailability, lack of or loss of efficacy or pharmacokinetic or pharmacodynamic risks.

The term “upstream neighborhood gene”, as used herein, refers to a gene upstream of a primary neighborhood gene that may be located within the same insulated neighborhood as the primary neighborhood gene.

The term “signaling signature”, as used herein, refers to signaling interations within a gene signaling network associated with a genomic signaling center in an insulated neighborhood.

Described herein are compositions and methods for perturbation of GSCs or entire GSNs. Further described herein are compositions and methods for modulating gene expression in a cell by altering the binding profile of an ODSC. The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

The present invention is further illustrated by the following non-limiting examples.

### VI. Examples

### Example 1. Experimental Procedures

A. Erythroid Cell Culture

CD34⁺ cells were isolated from cord blood and peripheral blood (about 1×10⁶). Cells were expanded over 11 days to collect a total of 100×10⁶ cord blood cells and 25×10⁶ peripheral blood cells. The cells were conditionally immortalized as described below on the third day, differentiated in erythroid medium and collected on the seventh day (about 400×10⁶ cord blood CD34⁺ cells or about 100×10⁶ peripheral blood CD34⁺ cells).

B. Hepatocyte Cell Culture

Cryopreserved hepatocytes were cultured in plating media for 16 hours, transferred to maintenance media for 4 hours. Cultured on serum-free media for 2 hours, then a compound was added. The hepatocytes were maintained on the serum-free media for 16 hours prior to gene expression analysis. Primary Human Hepatocytes were stored in the vapor phase of a liquid nitrogen freezer (about −130° C.).

To seed the primary human hepatocytes, vials of cells were retrieved from the LN₂ freezer, thawed in a 37° C. water bath, and swirled gently until only a shyer of ice remains. Using a 10 ml serological pipet, cells were gently pipetted out of the vial and gently pipetted down the side of 50 mL conical tube containing 20 mL cold thaw medium. The vial was rinsed with about 1 mL of thaw medium, and the rinse was added to the conical tube. Up to 2 vials may be added to one tube of 20 mL thaw medium.

The conical tube(s) were gently inverted 2-3 times and centrifuged at 100 g for 10 minutes at 4° C. with reduced braking (e.g. 4 out of 9). The thaw medium slowly was slowly aspirated to avoid the pellet. 4 mL cold plating medium was added slowly down the side (8 mL if combined 2 vials to 1 tube), and the vial was inverted gently several times to resuspend cells.

Cells were kept on ice until 100 μl of well-mixed cells were added to 400 μl diluted Trypan blue and mixed by gentle inversion. They were counted using a hemocytometer (or Cellometer), and viability and viable cells/mL were noted. Cells were diluted to a desired concentration and seeded on collagen 1-coated plates. Cells were pipetted slowly and gently onto plate, only 1-2 wells at a time. The remaining cells were mixed in the tubes frequently by gentle inversion. Cells were seeded at about 8.5×10⁶ cells per plate in 6 mL cold plating medium (10 cm). Alternatively, 1.5×10⁶ per well for a 6-well plate (1 mL medium/well); 7×10⁵ per well for 12-well plate (0.5 mL/well); or 3.75×10⁵ per well fora 24-well plate (0.5 mL/well)

After all cells and medium were added to the plate, the plate was transferred to an incubator (37° C., 5% CO2, about 90% humidity) and rocked forwards and backwards, then side to side several times each to distribute cells evenly across the plate or wells. The plate(s) were rocked again every 15 minutes for the first hour post-plating. About 4 hours post-plating (or first thing the morning if cells were plated in the evening), cells were washed once with PBS and complete maintenance medium was added. The primary human hepatocytes were maintained in the maintenance medium and transferred to fresh medium daily.

C. Starvation and Compound Treatment of Hepatocytes

Two to three hours before treatment, cells cultured as described above were washed with PBS and the medium was changed to either: fresh maintenance medium (complete) or modified maintenance medium 4b.

Compound stocks were prepared at 1000× final concentration and added in a 2-step dilution to the medium to reduce risk of a compound precipitating out of solution when added to the cells, and to ensure reasonable pipetting volumes. One at a time, each compound was first diluted 10-fold in warm (about 37° C.) modified maintenance medium (initial dilution=1D), mixed by vortexing, and the ID was diluted 100-fold into the cell culture (e.g. 5.1 μl into 1 well of a 24-well plate containing 0.5 mL medium). The plate was mixed by carefully swirling and after all wells were treated and returned to the incubator overnight. If desired, separate plates/wells were treated with vehicle-only controls and/or positive controls. If using multi-well plates, controls were included on each plate. After about 18 hours, cells were harvested for further analysis, e.g., ChIP-seq, RNA-seq, ATAC-seq, etc.

D. Media Composition

The thaw medium contained 6 mL isotonic percoll and 14 mL high glucose DMEM (Invitrogen #11965 or similar). The plating medium contained 100 mL Williams E medium (Invitrogen #A1217601, without phenol red) and the supplement pack #CM3000 from ThermoFisher Plating medium containing 5 mL FBS, 10 μl dexamethasone, and 3.6 mL plating/maintenance cocktail. Stock trypan blue (0.4%, Invitrogen #15250) was diluted 1:5 in PBS.

The ThermoFisher complete maintenance medium contained supplement pack #CM4000 (1 μl dexamethasone and 4 mL maintenance cocktail) and 100 mL Williams E (Invitrogen #A1217601, without phenol red).

The modified maintenance media had no stimulating factors (dexamethasone, insulin, etc.), and contained 100 mL Williams E (Invitrogen #A1217601, without phenol red), 1 mL L-Glutamine (Sigma #G7513) to 2 mM, 1.5 mL HEPES (VWR #J848) to 15 mM, and 0.5 mL penicillin/streptomycin (Invitrogen #15140) to a final concentration of 50U/mL each.

E. DNA Purification

DNA purification was conducted as described in Ji et al., PNAS 112(12):3841-3846 (2015) Supporting Information, which is hereby incorporated by reference in its entirety. One milliliter of 2.5 M glycine was added to each plate of fixed cells and incubated for 5 minutes to quench the formaldehyde. The cells were washed twice with PBS. The cells were pelleted at 1,300 g for 5 minutes at 4° C. Then, 4×10⁷ cells were collected in each tube. The cells were lysed gently with 1 mL of ice-cold Nonidet P-40 lysis buffer containing protease inhibitor on ice for 5 minutes (buffer recipes are provided below). The cell lysate was layered on top of 2.5 volumes of sucrose cushion made up of 24% (wt/vol) sucrose in Nonidet P-40 lysis buffer. This sample was centrifuged at 18,000 g for 10 minutes at 4° C. to isolate the nuclei pellet (the supernatant represented the cytoplasmic fraction). The nuclei pellet was washed once with PBS/1 mM EDTA. The nuclei pellet was resuspended gently with 0.5 mL glycerol buffer followed by incubation for 2 minutes on ice with an equal volume of nuclei lysis buffer. The sample was centrifuged at 16,000 g for 2 minutes at 4° C. to isolate the chromatin pellet (the supernatant represented the nuclear soluble fraction). The chromatin pellet was washed twice with PBS/1 mM EDTA. The chromatin pellet was stored at −80° C.

The Nonidet P-40 lysis buffer contained 10 mM Tris.HCl (pH 7.5), 150 mM NaCl, and 0.05% Nonidet P-40. The glycerol buffer contained 20 mM Tris.HCl (pH 7.9), 75 mM NaCl, 0.5 mM EDTA, 0.85 mM DTT, and 50% (vol/vol) glycerol. The nuclei lysis buffer contained 10 mM Hepes (pH 7.6), 1 mM DTT, 7.5 mM MgCl2, 0.2 mM EDTA, 0.3 M NaCl, 1 M urea, and 1% Nonidet P-40.

F. Chromatin Immunoprecipitation Sequencing (ChIP-Seq)

ChIP-seq was performed using the following protocol for primary hepatocytes, HepG2 cells, and erythroid cells to determine the composition and confirm the location of genomic signaling centers, including occupancy-dependent signaling centers.

i. Cell Cross-Linking

2×10⁷ cells were used for each run of ChIP-seq. Two ml of fresh 11% formaldehyde (FA) solution was added to 20 ml media on 15 cm plates to reach a 1.1% final concentration. Rates were swirled briefly and incubated at room temperature (RT) for 15 minutes. At the end of incubation, the FA was quenched by adding 1 ml of 2.5M Glycine to plates and incubating for 5 minutes at RT. The media was discarded to a 1L beaker, and cells were washed twice with 20 ml ice-cold PBS. PBS (10 ml) was added to plates, and cells were scraped off the plate. The cells were transferred to 15 ml conical tubes, and the tubes were placed on ice. Plates were washed with an additional 4 ml of PBS and combined with cells in 15 ml tubes. Tubes were centrifuged for 5 minutes at 1,500 rpm at 4° C. in a tabletop centrifuge. PBS was aspirated, and the cells were flash frozen in liquid nitrogen. Pellets were stored at −80° C. until ready to use.

ii. Pre-Block Magnetic Beads

Thirty μl Protein G beads (per reaction) were added to a 1.5 ml Protein LoBind Eppendorf tube. The beads were collected by magnet separation at RT for 30 seconds. Beads were washed 3 times with 1 ml of blocking solution by incubating beads on a rotator at 4° C. for 10 minutes and collecting the beads with the magnet. Five pg of an antibody was added to the 250 μl of beads in block solution. The mix was transferred to a clean tube, and rotated 0/N at 4° C. On the next day, buffer containing antibodies was removed, and beads were washed 3 times with 1.1 ml blocking solution by incubating beads on a rotator at 4° C. for 10 minutes and collecting the beads with the magnet. Beads were resuspended in 50 μl of block solution and kept on ice until ready to use.

iii. Cell Lysis, Genomic Fragmentation, and Chromatin Immunoprecipitation

Complete® protease inhibitor cocktail was added to lysis buffer 1 (LB1) before use. One tablet was dissolved in 1 ml of H₂O for a 50× solution. The cocktail was stored in aliquots at −20° C. Cells were resuspended in each tube in 8 ml of LB1 and incubated on a rotator at 4° C. for 10 minutes. Nuclei were spun down at 1,350 g for 5 minutes at 4° C. LB1 was aspirated, and cells were resuspended in each tube in 8 ml of LB2 and incubated on a rotator at 4° C. for 10 minutes.

A Covaris® E220evouton™ ultrasonicator was programmed per the manufacturer's recommendations for high cell numbers. HepG2 and erythroid cells were sonicated for 12 minutes, and primary hepatocyte samples were sonicated for 10 minutes. Lysates were transferred to dean 1.5 ml Eppendorf tubes, and the tubes were centrifuged at 20,000 g for 10 minutes at 4° C. to pellet debris. The supernatant was transferred to a 2 ml Protein LoBind Eppendorf tube containing pre-blocked Protein G beads with pre-bound antibodies. Fifty μl of the supernatant was saved as input. Input material was kept at −80° C. until ready to use. Tubes were rotated with beads overnight at 4° C.

iv. Wash, Elution, and Cross-Link Reversal

All washing steps were performed by rotating tubes for 5 minutes at 4° C. The beads were transferred to clean Protein LoBind Eppendorf tubes with every washing step. Beads were collected in 1.5 ml Eppendorf tube using a magnet. Beads were washed twice with 1.1 ml of sonication buffer. The magnetic stand was used to collect magnetic beads. Beads were washed twice with 1.1 ml of wash buffer 2, and the magnetic stand was used again to collect magnetic beads. Beads were washed twice with 1.1 ml of wash buffer 3. All residual Wash buffer 3 was removed, and beads were washed once with 1.1 ml TE+0.2% Triton X-100 buffer. Residual TE+0.2% Triton X-100 buffer was removed, and beads were washed twice with TE buffer for 30 seconds each time. Residual TE buffer was removed, and beads were resuspended in 300 μl of ChIP elution buffer. Two hundred fifty μl of ChIP elution buffer was added to 50 μl of input, and the tubes were rotated with beads 1 hour at 65° C. Input sample was incubated overnight at 65° C. oven without rotation. Tubes with beads were placed on a magnet, and the eluate was transferred to a fresh DNA LoBind Eppendorf tube. The eluate was incubated overnight at 65° C. oven without rotation

v. Chromatin Extraction and Precipitation

Input and immunoprecipitant (IP) samples were transferred to fresh tubes, and 300 μl of TE buffer was added to IP and Input samples to dilute SDS. RNase A (20 mg/ml) was added to the tubes, and the tubes were incubated at 37° C. for 30 minutes. Following incubation, 3 μl of 1M CaCl2 and 7 μl of 20 mg/ml Proteinase K were added, and incubated 1.5 hours at 55° C. MaXtract High Density 2 ml gel tubes (Qiagen) were prepared by centrifugation at full speed for 30 seconds at RT. Six hundred μl of phenol/chloroform/isoamyl alcohol was added to each proteinase K reaction and transferred in about 1.2 ml mixtures to the MaXtract tubes. Tubes were spun at 16,000 g for 5 minutes at RT. The aqueous phase was transferred to two dean DNA LoBind tubes (300 μl in each tube), and 1.5 μl glycogen, 30 μl of 3M sodium acetate, and 900 μl ethanol were added. The mixture was precipitated overnight at −20° C. or for 1 hour at −80° C., and spun down at maximum speed for 20 minutes at 4° C. The ethanol was removed, and pellets were washed with 1 ml of 75% ethanol by spinning tubes down at maximum speed for 5 minutes at 4° C. Remnants of ethanol were removed, and pellets were dried for 5 min at RT. Twenty-five μl of H₂O was added to each immunoprecipitant (IP) and input pellet, left standing for 5 minutes, and vortexed briefly. DNA from both tubes was combined to obtain 50 μl of IP and 50 μl of input DNA for each sample. One μl of this DNA was used to measure the amount of pulled down DNA using Qubit dsDNA HS assay (ThermoFisher, #Q32854). The total amount of immunoprecipitated material ranged from several ng (for TFs) to several hundred ng (for chromatin modifications). Six μl of DNA was analyzed using qRT-PCR to determine enrichment. The DNA was diluted if necessary. If enrichment was satisfactory, the rest was used for library preparation for DNA sequencing.

vi. Library Preparation for DNA Sequencing

Libraries were prepared using NEBNext Ultra 11 DNA library prep kit for Illumina (NEB, #E7645) using NEBNext Multiplex Oligos for Illumina (NEB, #6609S) according to manufacturer's instructions with the following modifications. The remaining ChIP sample (about 43 μl) and 1 μg of input samples for library preparations were brought up the volume of 50 μl before the End Repair portion of the protocol. End Repair reactions were run in a PCR machine with a heated lid in a 96-well semi-skirted PCR plate (ThermoFisher, #AB1400) sealed with adhesive plate seals (ThermoFisher, #AB0558) leaving at least one empty well in-between different samples. Undiluted adapters were used for input samples, 1:10 diluted adapters for 5-100 ng of ChIP material, and 1:25 diluted adapters for less than 5 ng of ChIP material. Ligation reactions were run in a PCR machine with the heated lid off. Adapter ligated DNA was transferred to clean DNA LoBind Eppendorf tubes, and the volume was brought to 96.5 μl using H₂O.

200-600 bp ChIP fragments were selected using SPRIselect magnetic beads (Beckman Coulter, #B23317). Thirty μl of the beads were added to 96.5 μl of ChIP sample to bind fragments that are longer than 600 bp. The shorter fragments were transferred to a fresh DNA LoBind Eppendorf tube. Fifteen μl of beads were added to bind the DNA longer than 200 bp, and beads were washed with DNA twice using freshly prepared 75% ethanol. DNA was eluted using 17 μl of 0.1×TE buffer. About 15 μl of DNA was collected.

Three μl of size-selected Input sample and all (15 μl) of the ChIP sample was used for PCR. The amount of size-selected DNA was measured using a Qubit dsDNA HS assay. PCR was run for 7 cycles of for Input and ChIP samples with about 5-10 ng of size-selected DNA, and 12 cycles with less than 5 ng of size-selected DNA. One-half of the PCR product (25 μl) was purified with 22.5 μl of AMPure XP beads (Beckman Coulter, #A63880) according to the manufacturer's instructions. PCR product was eluted with 17 μl of 0.1×TE buffer, and the amount of PCR product was measured using Qubit dsDNA HS assay. An additional 4 cycles of PCR were run for the second half of samples with less than 5 ng of PCR product, DNA was purified using 22.5 μl of AMPure XP beads. The concentration was measured to determine whether there was an increased yield. Both halves were combined, and the volume was brought up to 50 μl using H₂O.

A second round of purifications of DNA was run using 45 μl of AMPure XP beads in 17 μl of 0.1×TE, and the final yield was measured using Qubit dsDNA HS assay. This protocol produces from 20 ng to 1 mg of PCR product. The quality of the libraries was verified by diluting 1 μl of each sample with H₂O if necessary using the High Sensitivity BioAnalyzer DNA kit (Agilent, #5067-4626) based on manufacturer's recommendations.

vii. Reagents

11% Formaldehyde Solution (50 mL) contained 14.9 ml of 37% formaldehyde (final conc. 11%), 1 ml of 5M NaCl (final conc. 0.1 M), 100 μl of 0.5M EDTA (pH 8) (final conc. 1 mM), 50 μl of 0.5M EGTA (pH 8) (final conc. 0.5 mM), and 2.5 ml 1M Hepes (pH 7.5) (final conc. 50 mM).

Block Solution contained 0.5% BSA (w/v) in PBS and 500 mg BSA in 100 ml PBS. Block solution may be prepared up to about 4 days prior to use.

Lysis buffer 1 (LB1) (500 ml) contained 25 ml of 1 M Hepes-KOH, pH 7.5; 14 ml of 5M NaCl; 1 ml of 0.5M EDTA, pH 8.0; 50 ml of 100% Glycerol solution; 25 ml of 10% NP-40; and 12.5 ml of 10% Triton X-100. The pH was adjusted to 7.5. The buffer was sterile-filtered, and stored at 4° C. The pH was re-checked immediately prior to use.

Lysis buffer 2 (LB2) (1000 ml) contained 10 ml of 1 M Tris-HCL, pH 8.0; 40 ml of 5 M NaCl; 2 ml of 0.5M EDTA, pH 8.0; and 2 ml of 0.5M EGTA, pH 8.0. The pH was adjusted to 8.0. The buffer was sterile-filtered, and stored at 4° C. The pH was re-checked immediately prior to use.

Sonication buffer (500 ml) contained 25 ml of 1M Hepes-KOH, pH 7.5; 14 ml of 5M NaCl; 1 ml of 0.5M EDTA, pH 8.0; 50 ml of 10% Triton X-100; 10 ml of 5% Na-deoxycholate; and 5 ml of 10% SDS. The pH was adjusted to 7.5. The buffer was sterile-filtered, and stored at 4° C. The pH was re-checked immediately prior to use.

Proteinase inhibitors were included in the LB1, LB2, and Sonication buffer.

Wash Buffer 2 (500 ml) contained 25 ml of 1M Hepes-KOH, pH 7.5; 35 ml of 5M NaCl; 1 ml of 0.5M EDTA, pH 8.0; 50 ml of 10% Triton X-100; 10 ml of 5% Na-deoxycholate; and 5 ml of 10% SDS. The pH was adjusted to 7.5. The buffer was sterile-filtered, and stored at 4° C. The pH was re-checked immediately prior to use.

Wash Buffer 3 (500 ml) contained 10 ml of 1M Tris-HCL, pH 8.0; 1 ml of 0.5M EDTA, pH 8.0; 125 ml of 1M LiCl solution; 25 ml of 10% NP-40; and 50 ml of 5% Na-deoxycholate. The pH was adjusted to 8.0. The buffer was sterile-filtered, and stored at 4° C. The pH was re-checked immediately prior to use.

ChIP elution Buffer (500 ml) contained 25 ml of 1 M Tris-HCL, pH 8.0; 10 ml of 0.5M EDTA, pH 8.0; 50 ml of 10% SDS; and 415 ml of ddH2O. The pH was adjusted to 7.5. The buffer was sterile-filtered, and stored at 4° C. The pH was re-checked immediately prior to use.

G. Analysis of ChIP-Seq Results

AM obtained reads from each sample were trimmed using trim_galore 0.4.1 requiring a Phred score≥20 and a read length≥30. The trimmed reads were mapped against the human genome (hg19 build) using Bowtie (version 1.1.2) with the parameters: -v 2 -m 1 -S -t. AM unmapped reads, non-uniquely mapped reads and PCR duplicates were removed. AM the ChIP-seq peaks were identified using MACS2 with the parameters: -q 0.01-SPMR. The ChIP-seq signal was visualized in the UCSC genome browser. ChIP-seq peaks that are at least 2 kb away from annotated promoters (RefSeq, Ensemble and UCSC Known Gene databases combined) were selected as distal ChIP-seq peaks.

H. RNA-Seq

This protocol is a modified version of the following protocols: MagMAX mirVana Total RNA Isolation Kit User Guide (Applied Biosystems #MAN0011131 Rev B.0), NEBNext Poly(A) mRNA Magnetic Isolation Module (E7490), and NEBNext Ultra Directional RNA Library Prep Kit for Illumina (E7420) (New England Biosystems #E74901).

The MagMAX mirVana kit instructions (the section titled “Isolate RNA from cells” on pages 14-17) were used for isolation of total RNA from cells in culture. Two hundred μl of Lysis Binding Mix was used per well of the multiwell plate containing adherent cells (usually a 24-well plate).

For mRNA isolation and library prep, the NEBNext Poly(A) mRNA Magnetic Isolation Module and Directional Prep kit was used. RNA isolated from cells above was quantified, and prepared in 500 μg of each sample in 50 μl of nuclease-free water. This protocol may be run in microfuge tubes or in a 96-well plate.

The 80% ethanol was prepared fresh, and all elutions are done in 0.1×TE Buffer. For steps requiring Ampure XP beads, beads were at room temperature before use. Sample volumes were measured first and beads were pipetted. Section 1.9B (not 1.9A) was used for NEBNext Multiplex Oligos for Illumina (#E6609). Before starting the PCR enrichment, cDNA was quantified using the Qubit (DNA High Sensitivity Kit, ThermoFisher #Q32854). The PCR reaction was run for 12 cycles.

After purification of the PCR Reaction (Step 1.10), the libraries were quantified using the Qubit DNA High Sensitivity Kit. 1 μl of each sample were diluted to 1-2 ng/μl to run on the Bioanalyzer (DNA High Sensitivity Kit, Agilent #5067-4626). If Bioanalyzer peaks were not clean (one narrow peak around 300 bp), the AMPure XP bead cleanup step was repeated using a 0.9× or 1.0× beads:sample ratio. Then, the samples were quantified again with the Qubit, and run again on the Bioanalyzer (1-2 ng/μl).

Nuclear RNA from INTACT-purified nuclei or whole neocortical nuclei was converted to cDNA and amplified with the Nugen Ovation RNA-seq System V2. Libraries were sequenced using the Illumina HiSeq 2500.

I. RNA-Seq Data Analysis

All obtained reads from each sample were mapped against the human genome (hg19 build) using STAR_2.5.2 b, which allows mapping across splice sites by reads segmentation (Dobin et al., Bioinformatics (2012) 29 (1):15-21, which is hereby incorporated by reference in its entirety). The uniquely mapped reads were subsequently assembled into transcripts guided by reference annotation (RefSeq gene models) (Pruitt et al., Nucleic Acids Res. 2012 January; 40(Database issue): D130-D135, which is incorporated by reference in its entirety) with Cuffnorm v2.2.1 (Trapnell et al., Nature Protocols 7, 562-578 (2012), which is hereby incorporated by reference in its entirety). The expression level of each gene was quantified with normalized FPKM (fragments per kilobase of exon per million mapped fragments). The differentially expressed genes were called using Cuffdiff v2.2.1 with q value<0.01 and log 2fold change>=1 or <=−1.

J. ATAC-Seq

Hepatocytes were seeded overnight, then the serum and other factors were removed. After 2-3 hours, the cells were treated with the compound and incubated overnight. The cells were harvested and the nuclei were prepared for the transposition reaction. 50,000 bead bound nuclei were transposed using Tn5 transposase (Illumina FC-121-1030) as described in Mo et al., 2015, Neuron 86, 1369-1384, which is hereby incorporated by reference in its entirety. After 9-12 cycles of PCR amplification, libraries were sequenced on an Illumina HiSeq 2000. PCR was performed using barcoded primers with extension at 72° C. for 5 minutes, PCR, then the final PCR product was sequenced.

All obtained reads from each sample were trimmed using trim_galore 0.4.1 requiring Phred score 20 and read length 30 for data analysis. The trimmed reads were mapped against the human genome (hg19 build) using Bowtie2 (version 2.2.9) with the parameters: -t -q -N 1 -L 25 -X 2000 no-mixed no-discordant. All unmapped reads, non-uniquely mapped reads and PCR duplicates were removed. All the ATAC-seq peaks were called using MACS2 with the parameters -nolambda -nomodel -q 0.01 --SPMR. The ATAC-seq signal was visualized in the UCSC genome browser. ATAC-seq peaks that were at least 2 kb away from annotated promoters (RefSeq, Ensemble and UCSC Known Gene databases combined) were selected as distal ATAC-seq peaks.

K. qRT-PCR

qRT-PCR was performed as described in North et al., PNAS, 107(40) 17315-17320 (2010), which is hereby incorporated by reference in its entirety. qRT-PCR was performed with cDNA using the iQ5 Multicolor rtPCR Detection system from BioRad with 60° C. annealing.

Analysis of the fold changes in expression as measured by qRT-PCR were performed using the technique below. The control was DMSO, and the treatment was the selected compound (CPD). The internal control was GAPDH or B-Actin, and the gene of interest is the target. First, the averages of the 4 conditions were calculated for normalization: DMSO:GAPDH, DMSO:Target, CPD: GAPDH, and CPD:Target. Next, the ACT of both control and treatment were calculated to normalize to internal control (GAPDH) using (DMSO:Target)−(DMSO:GAPDH)=ΔCT control and (CPD:Target)−(CPD: GAPDH)=ΔCT experimental. Then, the MOT was calculated by ΔCT experimental−ΔCT control. The Expression Fold Change was calculated by 2-(MOT) (2-fold expression change was shown by RNA-Seq results provided herein).

L. Conditional Immortalization of Hematopoietic Cells

Conditional immortalization of hematopoietic cells was performed as described in Hirose et al., 2013, Stem Cell Reports, Vol. 1:499-508, the contents of which are hereby incorporated by reference in their entirety. For the immortalization of hematopoietic cells, human hematopoietic progenitor (CD34+) cells were purchased from commercial vendors as purified cells or isolated from peripheral blood. Hematopoietic progenitor cells, were collected on day 11 of culture, and 5×10⁴ cells were suspended in erythroid differentiation medium (Stemcell Technologies, Canada). Then, the cells were transduced with viral supernatant for DOX-inducible c-MYC, or BCL-XL, or a combination of c-MYC and BCL-XL (multiplicity of infection: MOI=20), E6/E7, hTERT, or SV40T system. Viral transduction was achieved using the retronectin-transduction protocol (Clontech). Cells were cultured in the presence of 1 μg/ml DOX.

Erythroid cells were cultured in erythroid differentiation media supplemented with antibiotics blasticidin and/or puromycin and 1 μg/ml DOX. The cells were cultivated for an additional 12 days to assure all cells expressed the desired transgenes required for conditional immortalization.

M. Conditional Immortalization of Hepatocytes

Cryopreserved hepatocytes were seeded on collagen-coated culture plates at a density of 30,000 cells/cm² in Hepatocyte growth medium (HGM) containing William's E basal medium supplemented with 100 μM dexamethasone, 3.75 g/L Bovine Serum Albumin (BSA), 1× insulin, transferrin and selenium (ITS), 1% penicillin-streptomycin, and 10 ng/mL oncostatin M (OSM) (Sigma-Aldrich, St. Louis, US). After 24 hours, the cells were transduced with viral particles containing HPV E6 and E7 genes. The cells were cultured for an additional 3 to 8 weeks in the presence of OSM. The medium was replaced every 2 to 3 days. Ten days after the transduction, proliferating colonies were observed in the transduced cell cultures. For stabilization of the epithelial phenotype of the cells, cultures were treated with 2 to 10 μM of MEK1/2 inhibitor U0126 (Sigma-Aldrich, St. Louis, US) for 1 to 3 weeks. Colonies of proliferating hepatocytes maintaining their epithelial phenotype were selected by trypsinization and re-seeded at a density of 20,000 cells/cm² and sub-cultured. The number of population doublings (PD) was calculated at each passage. The expression of CYP450, HPV E6, and HPV E7 was evaluated at population doubling number 19. When the colonies were expanded to 120×10⁶ cells, the hepatocytes were cryopreserved in HGM containing 20% FBS and 10% DMSO (See, Levy et al., 2015, Nat Biotechnol; 33(12):1264-1271, the contents of which are hereby incorporated by reference in their entireties).

N. Generating Stable Knockdown Cell Lines

293T cells were transfected over 4 days using pLKO (constitutive, U6 promoter), pTRIPZ (dox inducible, minimal cytomegalovirus, red fluorescent protein [RFP]), or pSMART (dox inducible, EF1alpha RFP, or phosphoglycerate kinase green fluorescent protein [PGK GFP]) plasmids to generate shRNA lentiviruses.

Conditionally immortalized cord blood CD34+ cells were transduced with the shRNA lentiviruses using the retronectin-transduction protocol (Clontech). Puromycin selection was performed 24-48 hours post-transduction. Cells were counted to monitor cell viability. After 12 days, which allowed for a large biomass, knockdown efficiency was analyzed. qRT-PCR was used to identify changes in mRNA, and Western blot was used to determine levels of protein depletion.

Cryopreserved CD34+ cells (cord blood, peripheral blood or bone marrow derived) were thawed from cryovials (typically 1×10⁶ or 5×10⁶ cells/vial) at 37° C. until completely thawed. Cells were then grown to a density of 1×10⁶ cells/mL for a period of 11 days in Hematopoietic Stem Cell expansion media DXF (Promocell) for the expansion of hematopoietic progenitor cells. Expanded CD34+ progenitor cells were then grown for an additional 3 days for erythroid differentiation in SFEMII medium (Stem Cell Technologies) supplemented with the Erythroid Expansion supplement (Stem Cell Technologies). Cells can be efficiently cryopreserved at this step and are ready for viral transduction when virus is prepared.

The vector and the shRNA of interest were selected from the plasmid list of Table 23. The SEQ ID NOs represent the nucleic acid sequence targets of the shRNA.

TABLE 23
Plasmid list

| SEQ ID NO | Vector | Clone ID | Target | Modification |
|---|---|---|---|---|
| 32561 | pZIP-TRE3G-ZsGreen-PURO | 3_2 = sh1 | RPS19 | ULTRA shRNA KD |
| 32562 | pZIP-TRE3G-ZsGreen-PURO | 4_3 = sh2 | RPS19 | ULTRA shRNA KD |
| 32563 | pZIP-TRE3G-ZsGreen-PURO | 5_4 = sh3 | RPS19 | ULTRA shRNA KD |
| 32564 | pZIP-TRE3G-ZsGreen-PURO | 3382846 = sh3 | p53 | ULTRA shRNA KD |
| 32565 | pZIP-TRE3G-ZsGreen-PURO | 3382844 = sh2 | p53 | ULTRA shRNA KD |
| 32566 | pZIP-TRE3G-ZsGreen-PURO | 3382843 = Sh1 | p53 | ULTRA shRNA KD |
| 32567 | pLKO_4 | sh1 | RPL11 | shRNA KD |
| 32568 | pLKO_5 | sh2 | RPL11 | shRNA KD |
| 32569 | pLKO_6 | sh3 | RPL11 | shRNA KD |
| 32570 | pLKO_7 | sh1 | RPS19 | shRNA KD |
| 32571 | pLKO_8 | sh4 | RPL11 | shRNA KD |
| 32572 | pLKO_9 | sh2 | RPS19 | shRNA KD |
| 32573 | pLKO_10 | sh5 | RPL11 | shRNA KD |
| 32574 | pLKO_11 | sh3 | RPS19 | shRNA KD |
| 32575 | pLKO_16 | sh4 | RPS19 | shRNA KD |
| 32576 | pLKO_18 | sh5 | RPS19 | shRNA KD |
| 32577 | gRNA | NA | RPS19_1 | KO |
| 32578 | gRNA | NA | RPS19_2 | KO |
| 32579 | pLVX-TetOne-bla-RPL11_shr | NA | RPL11 | Overexpression |
| 32580 | pLVX-TetOne-bla-RPS19_shr | NA | RPS19 | Overexpression |
| 32569 | pTRIPZ-puro-RPL11_sh1 | NA | RPL11 | shRNA KD |
| 32571 | pTRIPZ-puro-RPL11_sh2 | NA | RPL11 | shRNA KD |
| 32570 | pTRIPZ-puro-RPS19_sh1 | NA | RPS19 | shRNA KD |
| 32581 | pTRIPZ-puro-RPS19_sh2 | NA | RPS19 | shRNA KD |
| 32582 | pTRIPZ-puro-P53_sh1 | NA | p53 | shRNA KD |
| 32583 | pTRIPZ-tGFP.Trad-shNT | NA | NA | NA |
| 32583 | pGIPZ-tGFP.Trad-shNT | NA | NA | NA |
| 32584 | pLKO.1-tGFP.Trad-shNT | NA | NA | NA |
| 32570 | pGIPZ-tGFPtrad-RSP19_sh1 | NA | sh1 in pLKO and pTRIPZ | NA |
| 32570 | pLKO.1-tGFPtrad-RSP19_sh1 | NA | NA | NA |

Additional vectors that were used include those listed in Table 24. The table provides the Clone ID, Target, Modification, Tag, Inducible or Constitutive (I/C), and Selection method.

TABLE 24
Additional plasmids

| Vector | Target | Modification | Promoter | Tag | I/C | Selection |
|---|---|---|---|---|---|---|
| pTRIPZ-neo | NA | tGFPtrad | CMV | GFP | I | Neo |
| pGIPZ | NA | tGFPtrad | CMV | GFP | I | GFP |
| pGIPZ-tGFP.Trad | NA | tGFPtrad | CMV | GFP | C | GFP |
| pLKO.1-bla | Stuffer | none | U6 | NA | C | Bla |
| pLKO.1-hygrp | stuffer | none | U6 | NA | C | Hygro |
| pLKO.1-nep | Stuffer | none | U6 | NA | C | Neo |
| pLKO.1-purp | stuffer | none | U6 | NA | C | Puro |
| pLKO.1-tGFP.Trad:stuffer | NA | none | U6 | NA | C | GFP |
| pLKO_1 | p53 | shRNA KD | U6 | NA | C | Puro |
| pLKO_12 | p53 | shRNA KD | U6 | NA | C | Puro |
| pLKO_13 | p53 | shRNA KD | U6 | NA | C | Puro |
| pLKO_14 | p53 | shRNA KD | U6 | NA | C | Puro |
| pLKO_15 | GFP | shRNA KD | U6 | NA | C | Puro |
| pLKO_17 | p53 | shRNA KD | U6 | NA | C | Puro |
| pLKO_3 | NT | shRNA KD | U6 | NA | C | Puro |
| PLKO1 | NA | tGFPtrad -sorting | U6 | GFP | C | GFP |
| pLVX-EF1a-IRES-bla | NA | None | EF1a | NA | I | Bla |
| pLVX-EF1a-IRES-bla-DEST | Destination | None | EF1a | NA | I | Bla |
| pLVX-EF1a-IRES-hygro | NA | None | EF1a | NA | I | Hygro |
| pLVX-EF1a-IRES-hygro-DEST | Destination | None | EF1a | NA | I | Hygro |
| pLVX-EF1a-IRES-neo | NA | None | EF1a | NA | I | Neo |
| pLVX-EF1a-IRES-neo-DEST | Destination | None | EF1a | NA | I | Neo |
| pLVX-EF1a-IRES-puro | NA | None | EF1a | NA | I | Puro |
| pLVX-EF1a-IRES-puro-DEST | Destination | None | EF1a | NA | I | Puro |
| pLVX-TetOne | Myc | Overexpression | PGK | NA | I | Puro |
| pLVX-TetOne | BCLXL | Overexpression | PGK | NA | I | Blast |
| pLVX-TetOne | Myc | Overexpression | PGK | NA | I | Blast |
| pLVX-TetOne | BCLXL | Overexpression | PGK | NA | I | Puro |
| pLVX-TetOne-Bla | BCLXL/Myc | IRES | PGK | NA | I | Blast |
| pLVX-TetOne-Bla | eGFP | Control | | GFP | NA | Blast |
| pLVX-TetOne-Bla | BCLXL/Myc | T2A | PGK | NA | NA | Puro |
| pLVX-TetOne-Bla empty | Empty | Control | PGK | NA | NA | Bla |
| pLVX-TetOne-Bla-DEST | Destination | none | PGK | NA | NA | Bla |
| pLVX-TetOne-Bla-HPV16 E6/E7 | NA | HPV16 E6/E7 | PGK | NA | NA | Bla |
| pLVX-TetOne-Bla-hTERT | NA | hTERT | PGK | NA | NA | Bla |
| pLVX-TetOne-Bla-SV40T | NA | SV40T | PGK | NA | NA | Bla |
| pLVX-TetOne-Hygro | NA | none | PGK | NA | NA | Hygro |
| pLVX-TetOne-Hygro-DEST | Destination | none | PGK | NA | NA | Hygro |
| pLVX-TetOne-Puro | NA | none | PGK | NA | NA | Puro |
| pLVX-TetOne-Puro-DEST | Destination | none | PGK | NA | NA | Puro |
| pSMART | RPS19 | shRNA KD | EF1 alpha | RFP | NA | Puro |
| PSMART | RPS19 | shRNA KD | EF1 alpha | RFP | NA | Puro |
| PSMART | RPS19EFP-RFP | shRNA KD | EF1 alpha | RFP | NA | Puro |
| PSMART | NT | shRNA KD | EF1 alpha | RFP | NA | Puro |
| pSMART | NT | shRNA KD | EF1 alpha | GFP | NA | Puro |
| PSMART | RPS19_1 | shRNA KD | pGK | GFP | NA | Puro |
| PSMART | RPS19_2 | shRNA KD | PGK | GFP | NA | Puro |
| PSMART | NT | shRNA KD | pGK | GFP | NA | Puro |
| pTRIPZ-Neo | Empty | NA | CMV | RFP | NA | Neo |
| pTRIPZ-Puro-shNT | NT | NT | CMV | RFP | NA | Puro |
| pTRIPZ-tGFP.Trad | stutter | NA | NA | RFP | NA | NA |

Table 25 provides additional inserts to be used in any of the plasmids listed in Tables 23 and 24.

TABLE 25
Additional plasmid inserts

| | | Target | Antisense |
|---|---|---|---|
| Target | Plasmid insert | SEQ ID NO. | SEQ ID NO. |
| RPS19 | TRCN0000074913 | 32570 | 32590 |
| RPS19 | TRCN0000074914 | 32571 | 32591 |
| RPS19 | TRCN0000074915 | 32574 | 32592 |
| RPS19 | TRCN0000074916 | 32574 | 32593 |
| RPS19 | TRCN0000074917 | 32576 | 32594 |
| RPL11 | TRCN0000117712 | 32567 | 32595 |
| RPL11 | TRCN0000117713 | 32568 | 32596 |
| RPL11 | TRCN0000117714 | 32569 | 32597 |
| RPL11 | TRCN0000117715 | 32571 | 32598 |
| RPL11 | TRCN0000117716 | 32573 | 32599 |
| RPS14 | TRCN0000008640 | 32585 | 32600 |
| RPS14 | TRCN0000008641 | 32586 | 32601 |
| RPS14 | TRCN0000008642 | 32587 | 32602 |
| RPS14 | TRCN0000008643 | 32588 | 32603 |
| RPS14 | TRCN0000008644 | 32589 | 32604 |

In additional embodiments, RPS19_sh1 (ctacgatgagaactggttct; SEQ ID NO: 32605) is inserted into one of the plasmids listed in Tables 23 and 24.

293FT cells (Clontech) were transfected with 7 μg of pLVX-TetOne-Puro lentiviral vector combined with Lenti-X Packaging Single Shots (VSV-G) plasmids (Clontech). 293FT cells were transfected using the plasmid mixture. For the transfection of 293FT cells with shRNA, 7 μg of combined shRNA lentivector, VSV-G, and packaging plasmids were added to the cells using Fugene reagent and OPTIMEM media.

For both transgene and shRNA transfection, 48 hours after the transfection, the viral supernatant was collected and filtered through a 0.45 uM PVDF unit. The viral particles were concentrated by adding ⅓ total volume of Lenti-X Concentrator (Clontech). The mixture was mixed well by inversion and incubated for 1-2 hours at 4° C. After precipitation, the tubes were centrifuged at 4° C. for more than 45 minutes at 1,500 g to pellet the virus. Then, the supernatant was aspirated and the pellet was resuspended in PBS to a concentration of 25-50×. Plates were coated with retronectin and incubated for more than 4 hours at room temperature or overnight at 4° C., using the Retronectin-transduction protocol (Clontech) according to the manufacturer's instructions. Following retronectin incubation, the plates were blocked with 2% BSA/PBS for more than 30 minutes, and washed with PBS. Target cells were seeded onto the retronectin-coated plates and the appropriate amount of virus was added. Then the plates were spun at 2,500 rpm for 1-2 hours at RT. Cells were incubated at 37° C. for 72 hours following the infection and prior to selection.

For selection, antibiotic was added in the cell culture medium (about 5 μg/mL of PURO). Following the death of all the non-resistant cells, the surviving cells were collected for RNA and protein assay. Alternatively, cells were transduced with lentiviral vectors containing a GFP reporter (GFPtrad) expressed in the cell membrane. Cells expressing membrane bound GFP were magnetically enriched using a GFP specific antibody in conjuction with MACS cell separation (Miltenyi Biotec). This approach does not enrich the cell population expressing intracellular GFP; however, it enriched for cells expressing GFP on the cell membrane.

O. Knockdown Efficiency

For RNA extraction, 1×10⁶ cells were harvested in 500 μL of TRIZOL reagent (Thermo Scientific), and incubated for 5 minutes at room temperature. Then, 100 μL of chloroform was added to the cells, and the mixture was vortexed and then centrifuged at 12,000× gravity for 15 minutes. The aqueous phase was transferred to fresh tubes and one volume of 70% ethanol was added. The TRIZOL Plus RNA purification Kit (Thermo Scientific) was used for the remaining extraction protocol according to the manufacturer's instructions. For the conversion of RNA to cDNA the High Capacity cDNA RT kit (Thermo Scientific) was used according to the manufacturer's instructions. The cDNA was analyzed with qRT-PCR to determine the fold change of knockdown cells using Taqman Fast PCR mix and Applied Biosystems probes (Thermo Scientific).

P. Chromatin Interaction Analysis by Paired-End Tag Sequencing (ChIA-PET)

ChIA-PET is performed as previously described in Chepelev et al. (2012) Cell Res. 22, 490-503; Fullwood et al. (2009) Nature 462, 58-64; Goh et al. (2012) J. Vis. Exp., http://dx.doi.org/10.3791/3770; Li et al. (2012) Cell 148, 84-98; and Dowen et al. (2014) Cell 159, 374-387, which are each hereby incorporated by reference in their entireties. Briefly, embryonic stem (ES) cells (up to 1×10⁸ cells) are treated with 1% formaldehyde at room temperature for 20 minutes and then neutralized using 0.2M glycine. The crosslinked chromatin is fragmented by sonication to size lengths of 300-700 bp. The anti-SMC1 antibody (Bethyl, A300-055A) is used to enrich SMC1-bound chromatin fragments. A portion of ChIP DNA is eluted from antibody-coated beads for concentration quantification and for enrichment analysis using quantitative PCR. For ChIA-PET library construction ChIP DNA fragments are end-repaired using T4 DNA polymerase (NEB). ChIP DNA fragments are divided into two aliquots and either linker A or linker Bis ligated to the fragment ends. The two linkers differ by two nucleotides which are used as a nucleotide barcode (Linker A with CG; Linker B with AT). After linker ligation, the two samples are combined and prepared for proximity ligation by diluting in a 20 ml volume to minimize ligations between different DNA-protein complexes. The proximity ligation reaction is performed with T4 DNA ligase (Fermentas) and incubated without rocking at 22° C. for 20 hours. During the proximity ligation DNA fragments with the same linker sequence are ligated within the same chromatin complex, which generated the ligation products with homodimeric linker composition.

However, chimeric ligations between DNA fragments from different chromatin complexes could also occur, thus producing ligation products with heterodimeric linker composition. These heterodimeric linker products are used to assess the frequency of nonspecific ligations and were then removed.

i. Day 1

The cells are crosslinked as described for ChIP. Frozen cell pellets are stored in the −80° C. freezer until ready to use. This protocol requires at least 3×10⁸ cells frozen in six 15 ml Falcon tubes (50 million cells per tube). Six 100 μl Protein G Dynabeads (for each ChIA-PET sample) are added to six 1.5 ml Eppendorf tubes on ice. Beads are washed three times with 1.5 ml Block solution, and incubated end over end at 4° C. for 10 minutes between each washing step to allow for efficient blocking. Protein G Dynabeads are resuspended in 250 μl of Block solution in each of six tubes and 10 μg of SMC1 antibody (Bethyl A300-055A) is added to each tube. The bead-antibody mixes are incubated at 4° C. end-over-end overnight.

ii. Day 2

Beads are washed three times with 1.5 ml Block solution to remove unbound IgG and incubated end-over-end at 4° C. for 10 minutes each time. Smc1-bound beads are resuspended in 100 μl of Block solution and stored at 4° C. Final lysis buffer 1 (8 ml per sample) is prepared by adding 50× Protease inhibitor cocktail solution to Lysis buffer 1 (LB1) (1:50). Eight ml of Final lysis buffer 1 was added to each frozen cell pellet (8 ml per sample×6). The cells are thoroughly resuspended and thawed on ice by pipetting up and down. The cell suspension is incubated again end-over-end for 10 minutes at 4° C. The suspension is centrifuged at 1,350 g for 5 minutes at 4° C. Concurrently, Final lysis buffer 2 (8 ml per sample) is prepared by adding 50× Protease inhibitor cocktail solution to lysis buffer 2 (LB2) (1:50)

After centrifugation, the supernatant is discarded, and the nuclei are thoroughly resuspended in 8 ml Final lysis buffer 2 by pipetting up and down. The cell suspension is incubated end-over-end for 10 minutes at 4° C. The suspension is centrifuged at 1,350 g for 5 minutes at 4° C. During incubation and centrifugation, the Final sonication buffer (15 ml per sample) is prepared by adding 50× Protease inhibitor cocktail solution to sonication buffer (1:50). The supernatant is discarded, and the nuclei are fully resuspended in 15 ml Final sonication buffer by pipetting up and down. The nuclear extract is extracted to fifteen 1 ml Covaris Evolution E220 sonication tubes on ice. An aliquot of 10 μl is used to check the size of unsonicated chromatin on a gel.

A Covaris sonicator is programmed according to manufacturer's instructions (12 minutes per 20 million cells=12×15=3 hours). The samples are sequentially sequenced as described above. The goal is to break chromatin DNA to 200-600 bp. If sonication fragments are too big, false positives become more frequent. The sonicated nuclear extract is dispensed into 1.5 ml Eppendorf tubes. 1.5 ml samples are centrifuged at full speed at 4° C. for 10 minutes. Supernatant (SNE) is pooled into a new pre-cooled 50 ml Falcon tube, and brought to a volume of 18 ml with sonication buffer. Two tubes of 50 μl were taken as input and to check the size of fragments. 250 μl of ChIP elution buffer is added and reverse crosslinking occurs at 65° C. overnight in the oven After reversal of crosslinking, the size of sonication fragments is determined on a gel.

Three ml of sonicated extract is added to 100 μl Protein G beads with SMC1 antibodies in each of six clean 15 ml Falcon tubes. The tubes containing SNE-bead mix are incubated end-over-end at 4° C. overnight (14 to 18 hours)

iii. Day 3

Half the volume (1.5 ml) of the SNE-bead mix is added to each of six pre-chilled tubes and SNE is removed using a magnet. The tubes are sequentially washed as follows: 1) 1.5 ml of Sonication buffer is added, the beads are resuspended and rotated for 5 minutes at 4° C. for binding, then the liquid was removed (step performed twice); 2) 1.5 ml of high-salt sonication buffer is added, and the beads are resuspended and rotated for 5 minutes at 4° C. for binding, then the liquid is removed (step performed twice); 3) 1.5 ml of high-salt sonication buffer is added, and the beads are resuspended and rotated for 5 minutes at 4° C. for binding, then the liquid is removed (step performed twice); 4) 1.5 ml of LiCl buffer is added, and the cells are resuspended and incubated end-over-end for 5 minutes for binding, then the liquid is removed (step performed twice); 5) 1.5 ml of 1×TE+0.2% Triton X-100 is used to wash the cells for 5 minutes for binding, then the liquid is removed; and 1.5 ml of ice-cold TE Buffer is used to wash the cells for 30 seconds for binding, then the liquid is removed (step performed twice). Beads from all six tubes are sequentially resuspended in beads in one 1,000 ul tube of 1× ice-cold TE buffer.

ChIP-DNA is quantified using the following protocol. Ten percent of beads (by volume), or 100 μl, are transferred into a new 1.5 ml tube, using a magnet. Beads are resuspended in 300 μl of ChIP elution buffer and the tube is rotated with beads for 1 hour at 65° C. The tube with beads is placed on a magnet and the eluate was transferred to a fresh DNA LoBind Eppendorf tube. The eluate is incubated overnight at 65° C. oven without rotating. Immuno-precipitated samples are transferred to fresh tubes, and 300 μl of TE buffer is added to the immuno-precipitants and Input samples to dilute. Five μl of RNase A (20 mg/ml) is added, and the tube is incubated at 37° C. for 30 minutes.

Following incubation, 3 μl of 1M CaCl2 and 7 μl of 20 mg/ml Proteinase K is added to the tube and incubated 1.5 hours at 55° C. MaXtract High Density 2 ml gel tubes (Qiagen) were prepared by centrifuging them at full speed for 30 seconds at RT. 600 μl of phenol/chloroform/isoamyl alcohol is added to each proteinase K reaction. About 1.2 ml of the mixtures is transferred to the MaXtract tubes. Tubes are spun at 16,000 g for 5 minutes at RT. The aqueous phase is transferred to two clean DNA LoBind tubes (300 μl in each tube), and 1 μl glycogen, 30 μl of 3M sodium acetate, and 900 μl ethanol is added. The mixture is allowed to precipitate overnight at −20° C. or for 1 hour at −80° C.

The mixture is spun down at maximum speed for 20 minutes at 4° C., ethanol is removed, and the pellets are washed with 1 ml of 75% ethanol by spinning tubes down at maximum speed for 5 minutes at 4° C. All remnants of ethanol are removed, and pellets are dried for 5 minutes at RT. H₂O is added to each tube. Each tube is allowed to stand for 5 minutes, and vortexed briefly. DNA from both tubes is combined to obtain 50 μl of IP and 100 μl of Input DNA.

The amount of DNA collected is quantitated by ChIP using Qubit (Invitrogen #Q32856). One μl intercalating dye is combined with each measure 1 μl of sample. Two standards that come with the kit are used. DNA from only 10% of the beads is being measured. About 400 ng of chromatin in 900 μl of bead suspension is obtained with a good enrichment at enhancers and promoters as measured by qRT-PCR.

iv. Day 3 or 4

End-blunting of ChIP-DNA is performed on the beads using the following protocol. The remaining chromatin/beads are split by pipetting, and 450 μl of bead suspension is aliquoted into 2 tubes. Beads are collected on a magnet. Supernatant is removed, and then the beads are resuspended in the following reaction mix: 70 μl 10×NEB buffer 2.1 (NEB, M0203L), 7 μl 10 mM dNTPs, 615.8 μl dH₂O, and 7.2 μl of 3 U/μl T4 DNA Polymerase (NEB, M0203L). The beads are incubated at 37° C. with rotation for 40 minutes. Beads are collected with a magnet, then the beads are washed 3 times with 1 ml ice-cold ChIA-PET Wash Buffer (30 seconds per each wash).

On-Bead A-tailing was performed by preparing Klenow (3′ to 5′exo-) master mix as stated below: 70 μl 10×NEB buffer 2, 7 μl 10 mM dATP, 616 μl dH20, and 7 μl of 3 U/μl Klenow (3′ to 5″exo-) (NEB, M0212L). The mixture is incubated at 37° C. with rotation for 50 minutes. Beads are collected with a magnet, then beads are washed 3 times with 1 ml of ice-cold ChIA-PET Wash Buffer (30 seconds per each wash).

Linkers are thawed gently on ice. Linkers are mixed well with water gently by pipetting, then with PEG buffer, then gently vortexed. Then, 1394 μl of master mix and 6 μl of ligase is added per tube and mixed by inversion. Parafilm is put on the tube, and the tube is incubated at 16° C. with rotation overnight (at least 16 hours). The biotinylated linker was ligated to ChIP-DNA on beads by setting up the following reaction mix and adding reagents in order: 1110 μl dH₂O, 4 μl 200 ng/μl biotinylated bridge linker, 280 μl5×T4 DNA ligase buffer with PEG (Invitrogen), and 6 μl 30 U/μl T4 DNA ligase (Fermentas).

v. Day 5

Exonuclease lambda/Exonuclease 1 On-Bead digestion was performed using the following protocol. Beads were collected with a magnet and washed 3 times with 1 ml of ice-cold ChIA-PET Wash Buffer (30 seconds per each wash). The Wash buffer is removed from beads, then resuspended in the following reaction mix: 70 μl 10× lambda nuclease buffer (NEB, M0262L), 618 μl nuclease-free dH20, 6 μl 5 U/μl Lambda Exonuclease (NEB, M0262L), and 6 μl Exonuclease I (NEB, M0293L). The reaction is incubated at 37° C. with rotation for 1 hour. Beads are collected with a magnet, and beads are washed 3 times with 1 ml ice-cold ChIA-PET Wash Buffer (30 seconds per each wash).

Chromatin complexes are eluted off the beads by removing all residual buffer and resuspending the beads in 300 μl of ChIP elution buffer. The tube with beads is rotated 1 hour at 65° C. The tube is placed on a magnet and the eluate is transferred to a fresh DNA LoBind Eppendorf tube. The eluate is incubated overnight at 65° C. in an oven without rotating.

vi. Day 6

The eluted sample is transferred to a fresh tube and 300 μl of TE buffer is added to dilute the SDS. Three μl of RNase A (30 mg/ml) is added to the tube, and the mixture is incubated at 37° C. for 30 minutes. Following incubation, 3 μl of 1M CaCl2 and 7 μl of 20 mg/ml Proteinase K is added, and the tube is incubated again for 1.5 hours at 55° C. MaXtract High Density 2 ml gel tubes (Qiagen) are precipitated by centrifuging them at full speed for 30 seconds at RT. Six hundred μl of phenol/chloroform/isoamyl alcohol is added to each proteinase K reaction, and about 1.2 ml of the mixture is transferred to the MaXtract tubes. Tubes are spun at 16,000 g for 5 minutes at RT.

The aqueous phase is transferred to two clean DNA LoBind tubes (300 μl in each tube), and 1 μl glycogen, 30 μl of 3M sodium acetate, and 900 μl ethanol is added. The mixture is precipitated for 1 hour at −80° C. The tubes are spun down at maximum speed for 30 minutes at 4° C., and the ethanol is removed. The pellets are washed with 1 ml of 75% ethanol by spinning tubes down at maximum speed for 5 minutes at 4° C. Remnants of ethanol are removed, and the pellets are dried for 5 minutes at RT. Thirty μl of H₂O is added to the pellet and allowed to stand for 5 minutes. The pellet mixture is vortexed briefly, and spun down to collect the DNA.

Qubit and DNA High Sensitivity ChIP are performed to quantify and assess the quality of proximity ligated DNA products. About 120 ng of the product is obtained.

vii. Day 7

Components for Nextera tagmentation are then prepared. One hundred ng of DNA is divided into four 25 μl reactions containing 12.5 μl 2× Tagmentation buffer (Nextera), 1 μl nuclease-free dH₂O, 2.5 μl Tn5 enzyme(Nextera), and 9 μl DNA (25 ng). Fragments of each of the reactions are analyzed on a Bioanalyzer for quality control.

The reactions are incubated at 55° C. for 5 minutes, then at 10° C. for 10 minutes. Twenty-five μl of H₂O is added, and tagmented DNA is purified using Zymo columns. Three hundred fifty μl of Binding Buffer is added to the sample, and the mixture is loaded into a column and spun at 13,000 rpm for 30 seconds. The flow through is re-applied and the columns are spun again. The columns are washed twice with 200 μl of wash buffer and spun for 1 minute to dry the membrane. The column is transferred to a clean Eppendorf tube and 25 μl of Elution buffer is added. The tube is spun down for 1 minute. This step is repeated with another 25 μl of elution buffer. All tagmented DNA is combined into one tube.

ChIA-PETs are immobilized on Streptavidin beads using the following steps. 2×B&W Buffer (40 ml) is prepared as follows for coupling of nucleic acids: 400 μl 1M Tris-HCl pH 8.0 (10 mM final), 80 μl 1M EDTA (1 mM final), 16 ml 5M NaCl (2M final), and 23.52 ml dH₂O. 1×B&W Buffer (40 ml total) is prepared by adding 20 ml dH₂O to 20 ml of the 2×B&W Buffer.

MyOne Streptavidin Dynabeads M-280 are allowed to come to room temperature for 30 minutes, and 30 μl of beads are transferred to a new 1.5 ml tube. Beads are washed with 150 μl of 2×B&W Buffer twice. Beads are resuspended in 100 μl of iBlock buffer (Applied Biosystems), and mixed. The mixture is incubated at RT for 45 minutes on a rotator.

I-BLOCK Reagent is prepared to contain: 0.2% I-Block reagent (0.2 g), 1×PBS or 1×TBS (10 ml 10×PBS or 10×TBS), 0.05% Tween-20 (50 μl), and H₂O to 100 ml. 10×PBS and I-BLOCK reagent is added to H₂O, and the mixture is microwaved for 40 seconds (not allowed to boil), then stirred. Tween-20 is added after the solution is cooled. The solution remains opaque, but particles are dissolved. The solution is coded to RT for use.

During incubation of beads, 500 ng of sheared genomic DNA is added to 50 μl of H₂O and 50 μl of 2×B&W Buffer. When the beads finish incubating with the iBLOCK buffer, they are washed twice with 200 μl of 1×B&W buffer. The wash buffer is discarded, and 100 μl of the sheared genomic DNA is added. The mixture is incubated with rotation for 30 minutes at RT. The beads are washed twice with 200 μl of 1×B&W buffer. Tagmented DNA is added to the beads with an equal volume of 2×B&W buffer and incubated for 45 minutes at RT with rotation. The beads are washed 5 times with 500 μl of 2×SSC/0.5% SDS buffer (30 seconds each time) followed by 2 washes with 500 ml of 1×B&W Buffer and incubating each after wash for 5 minutes at RT with rotation. The beads are washed once with 100 μl elution buffer (EB) from a Qiagen Kit by resuspending beads gently and putting the tube on a magnet. The supernatant is removed from the beads, and they were resuspended in 30 μl of EB.

A paired end sequencing library is constructed on beads using the following protocol. Ten μl of beads are tested by PCR with 10 cycles of amplification. The 50 μl of the PCR mixture contains: 10 μl of bead DNA, 15 μl NPM mix (from Illumina Nextera kit), 5 μl of PPC PCR primer, 5 μl of Index Primer 1 (i7), 5 μl of Index Primer 2 (i5), and 10 μl of H20. PCR is performed using the following cycle conditions: denaturing the DNA at 72° C. for 3 minutes, then 10-12 cycles of 98° C. for 10 seconds, 63° C. for 30 seconds, and 72° C. for 50 seconds, and a final extension of 72° C. for 5 minutes. The number of cycles is adjusted to obtain about 300 ng of DNA total with four 25 μl reactions. The PCR product may be held at 4° C. for an indefinite amount of time.

The PCR product was cleaned-up using AMPure beads. Beads are allowed to come to RT for 30 minutes before using. Fifty μl of the PCR reaction is transferred to a new Low-Bind Tube and (1.8×volume) 90 μl of AMPure beads is added. The mixture is pipetted well and incubated at RT for 5 minutes. A magnet is used for 3 minutes to collect beads and remove the supernatant. Three hundred μl of freshly prepared 80% ethanol is added to the beads on the magnet, and the ethanol is carefully dicarded. The wash is repeated, and then all ethanol is removed. The beads are dried on the magnet rack for 10 minutes. Ten μl EB is added to the beads, mixed well, and incubated for 5 minutes at RT. The eluate is collected, and 1 μl of eluate is used for Qubit and Bioanalyzer.

The library is cloned to verify complexity using the following protocol. One μl of the library is diluted at 1:10. A PCR reaction is performed as described below. Primers that anneal to Illumina adapters are chosen (Tm=52.2° C.). The PCR reaction mixture (total volume: 50 μl) contains the following: 10 μl of 5×GoTaq buffer, 1 μl of 10 mM dNTP, 5 μl of 10 μM primer mix, 0.25 μl of GoTaq polymerase, 1 μl of diluted template DNA, and 32.75 μl of H20. PCR is performed using the following cycle conditions: denaturing the DNA at 95° C. for 2 minutes and 20 cycles at the following conditions: 95° C. for 60 seconds, 50° C. for 60 seconds, and 72° C. for 30 seconds with a final extension at 72° C. for 5 minutes. The PCR product may be held at 4° C. for an indefinite amount of time.

The PCR product is ligated with the pGEM® T-Easy vector (Promega) protocol. Five μl of 2×T4 Quick ligase buffer, 1 μl of pGEM®-Easy vector, 1 μl of T4 ligase, 1 μl of PCR product, and 2 μl of H₂O are combined to a total volume of 10 μl. The product is incubated for 1 hour at RT and 2 μl is used to transform Stellar competent cells. Two hundred μl of 500 μl of cells are plated in SOC media. The next day, 20 colonies are selected for Sanger sequencing using a T7 promoter primer. 60% clones had a full adapter, and 15% had a partial adapter.

viii. Reagents

Protein G Dynabeads for 10 samples are from Invitrogen Dynal, Cat #10003D. Block solution (50 ml) contains 0.25g BSA dissolved in 50 ml of ddH2O (0.5% BSA, w/v), and is stored at 4° C. for 2 days before use.

Lysis buffer 1 (LB1) (500 ml) contains 25 ml of 1M Hepes-KOH, pH 7.5; 14 ml of 5M NaCl; 1 ml of 0.5 M EDTA, pH 8.0; 50 ml of 100% Glycerol solution; 25 ml of 10% NP-40; and 12.5 ml of 10% Triton X-100. The pH is adjusted to 7.5. The buffer is sterile-filtered, and stored at 4° C. The pH is re-checked immediately prior to use. Lysis buffer 2 (LB2) (1000 ml) contains 10 ml of 1M Tris-HCL, pH 8.0; 40 ml of 5 M NaCl; 2 ml of 0.5 M EDTA, pH 8.0; and 2 ml of 0.5 M EGTA, pH 8.0. The pH is adjusted to 8.0. The buffer is sterile-filtered, and stored at 4° C. The pH is re-checked immediately prior to use.

Sonication buffer (500 ml) contains 25 ml of 1M Hepes-KOH, pH 7.5; 14 ml of 5M NaCl; 1 ml of 0.5 M EDTA, pH 8.0; 50 ml of 10% Triton X-100; 10 ml of 5% Na-deoxycholate; and 5 ml of 10% SDS. The buffer is sterile-filtered, and stored at 4° C. The pH is re-checked immediately prior to use. High-salt sonication buffer (500 ml) contains 25 ml of 1M Hepes-KOH, pH 7.5; 35 ml of 5M NaCl; 1 ml of 0.5 M EDTA, pH 8.0; 50 ml of 10% Triton X-100; 10 ml of 5% Na-deoxycholate; and 5 ml of 10% SDS. The buffer is sterile-filtered, and stored at 4° C. The pH is re-checked immediately prior to use.

LiCl wash buffer (500 ml) contains 10 ml of 1M Tris-HCL, pH 8.0; 1 ml of 0.5M EDTA, pH 8.0; 125 ml of 1M LiCl solution; 25 ml of 10% NP-40; and 50 ml of 5% Na-deoxycholate. The pH is adjusted to 8.0. The buffer is sterile-filtered, and stored at 4° C. The pH is re-checked immediately prior to use.

Elution buffer (500 ml) used to quantify the amount of ChIP DNA contains 25 ml of 1M Tris-HCL, pH 8.0; 10 ml of 0.5M EDTA, pH 8.0; 50 ml of 10% SDS; and 415 ml of ddH2O. The pH is adjusted to 8.0. The buffer is sterile-filtered, and stored at 4° C. The pH is re-checked immediately prior to use.

ChIA-PET Wash Buffer (50 ml) contains 500 μl of 1M Tris-HCl, pH 8.0 (final 10 mM); 100 μl of 0.5M EDTA, pH 8.0 (final 1 mM); 5 ml of 5M NaCl (final 500 mM); and 44.4 ml of dH₂O.

Q. HiChIP

Alternatively to ChIA-PET, HiChIP was used to analyze chromatin interactions and conformation. HiChIP requires fewer cells than ChIA-PET.

i. Cell Crosslinking

Cells were cross-linked as described in the ChIP protocol above. Crosslinked cells were either stored as pellets at −80° C. or used for HiChIP immediately after flash-freezing the cells.

ii. Lysis and Restriction

Fifteen million cross-linked cells were resuspended in 500 μL of ice-cold Hi-C Lysis Buffer and rotated at 4° C. for 30 minutes. For cell amounts greater than 15 million, the pellet was split in half for contact generation and then recombined for sonication. Cells were spun down at 2500g for 5 minutes, and the supernatant was discarded. The pelleted nuclei were washed once with 500 μL of ice-cold Hi-C Lysis Buffer. The supernatant was removed, and the pellet was resuspended in 100 μL of 0.5% SDS. The resuspension was incubated at 62° C. for 10 minutes, and then 285 μL of H₂O and 50 μL of 10% Triton X-100 were added to quench the SDS. The resuspension was mixed well, and incubated at 37° C. for 15 minutes. Fifty μL of 10×NEB Buffer 2 and 375 U of Mbol restriction enzyme (NEB, R0147) was added to the mixture to digest chromatin for 2 hours at 37° C. with rotation. For lower starting material, less restriction enzyme is used: 15 μL was used for 10-15 million cells, 8 μL for 5 million cells, and 4 μL for 1 million cells. Heat (62° C. for 20 minutes) was used to inactivate Mbol.

iii. Biotin Incorporation and Proximity Ligation

To fill in the restriction fragment overhangs and mark the DNA ends with biotin, 52 μL of fill-in master mix was reacted by combining 37.5 μL of 0.4 mM biotin-dATP (Thermo 19524016); 1.5 μL of 10 mM dCTP, dGTP, and dTTP; and 10 μL of 5U/μL DNA Polymerase 1, Large (Klenow) Fragment (NEB, M0210). The mixture was incubated at 37° C. for 1 hour with rotation.

Nine hundred forty-eight μL of ligation master mix was added. Ligation Master Mix contains 150 μL of 10×NEB T4 DNA ligase buffer with 10 mM ATP (NEB, B0202); 125 μL of 10% Triton X-100; 3 μL of 50 mg/mL BSA; 10 μL of 400 U/μL T4 DNA Ligase (NEB, M0202); and 660 μL of water. The mixture was incubated at room temperature for 4 hours with rotation. The nuclei were pelleted at 2500g for 5 minutes, and the supernatant was removed.

iv. Sonication

For sonication, the pellet was brought up to 1000 μL in Nuclear Lysis Buffer. The sample was transferred to a Covaris millitube, and the DNA was sheared using a Covaris® E220Evouton™ with the manufacturer recommended parameters. Each tube (15 million cells) was sonicated for 4 minutes under the following conditions: Fill Level 5; Duty Cycle 5%; PIP 140; and Cycles/Burst 200.

v. Preclearing, Immunoprecipitation, IP Bead Capture, and Washes

The sample was clarified for 15 minutes at 16,100g at 4° C. The sample is split into 2 tubes of about 400 μL each and 750 μL of ChIP Dilution Buffer is added. For the Smc1a antibody (Bethyl A300-055A), the sample is diluted 1:2 in ChIP Dilution Buffer to achieve an SDS concentration of 0.33%. 60 μL of Protein G beads were washed for every 10 million cells in ChIP Dilution Buffer. Amounts of beads (for preclearing and capture) and antibodies were adjusted linearly for different amounts of cell starting material. Protein G beads were resuspended in 50 μL of Dilution Buffer per tube (100 μL per HiChIP). The sample was rotated at 4° C. for 1 hour. The samples were put on a magnet, and the supernatant was transferred into new tubes. 7.5 μg of antibody was added for every 10 million cells, and the mixture was incubated at 4° C. overnight with rotation. Another 60 μL of Protein G beads for every 10 million cells in ChIP Dilution Buffer was added. Protein G beads were resuspended in 50 μL of Dilution Buffer (100 μL per HiChIP), added to the sample, and rotated at 4° C. for 2 hours. The beads were washed three times each with Low Salt Wash Buffer, High Salt Wash Buffer, and LiCl Wash Buffer. Washing was performed at room temperature on a magnet by adding 500 μL of a wash buffer, swishing the beads back and forth twice by moving the sample relative to the magnet, and then removing the supernatant

vi. ChIP DNA Elution

ChIP sample beads were resuspended in 100 μL of fresh DNA Elution Buffer. The sample beads were incubated at RT for 10 minutes with rotation, followed by 3 minutes at 37° C. with shaking. ChIP samples were placed on a magnet, and the supernatant was removed to a fresh tube. Another 100 μL of DNA Elution Buffer was added to ChIP samples and incubations were repeated. ChIP sample supernatants were removed again and transferred to a new tube. There was about 200 μL of ChIP sample. Ten μL of Proteinase K (20 mg/ml) was added to each sample and incubated at 55° C. for 45 minutes with shaking. The temperature was increased to 67° C., and the samples were incubated for at least 1.5 hours with shaking. The DNA was Zymo-purified (Zymo Research, #D4014) and eluted into 10 μL of water. Post-ChIP DNA was quantified to estimate the amount of Tn5 needed to generate libraries at the correct size distribution. This assumed that contact libraries were generated properly, samples were not over sonicated, and that material was robustly captured on streptavidin beads. SMC1 HiChIP with 10 million cells had an expected yield of post-ChIP DNA from 15 ng-50 ng. For libraries with greater than 150 ng of post-ChIP DNA, materials were set aside and a maximum of 150 ng was taken into the biotin capture step

vii. Biotin Pull-Down and Preparation for Illumina Sequencing

To prepare for biotin pull-down, 5 μL of Streptavidin C-1 beads were washed with Tween Wash Buffer. The beads were resuspended in 10 μL of 2× Biotin Binding Buffer and added to the samples. The beads were incubated at RT for 15 minutes with rotation. The beads were separated on a magnet, and the supernatant was discarded. The beads were washed twice by adding 500 μL of Tween Wash Buffer and incubated at 55° C. for 2 minutes while shaking. The beads were washed in 100 μL of 1× (diluted from 2×) TD Buffer. The beads were resuspended in 25 μL of 2×TD Buffer, 2.5 μL of Tn5 for each 50 ng of post-ChIP DNA, and water to a volume of 50 μL.

The Tn5 had a maximum amount of 4 μL. For example, for 25 ng of DNA transpose, 1.25 μL of Tn5 was added, while for 125 ng of DNA transpose, 4 μL of Tn5 was used. Using the correct amount of Tn5 resulted in proper size distribution. An over-transposed sample had shorter fragments and exhibited lower alignment rates (when the junction was close to a fragment end). An undertransposed sample has fragments that are too large to duster properly on an Illumina sequencer. The library was amplified in 5 cycles and had enough complexity to be sequenced deeply and achieve proper size distribution regardless of the level of transposition of the library.

The beads were incubated at 55° C. with interval shaking for 10 minutes. Samples were placed on a magnet, and the supernatant was removed. Fifty mM EDTA was added to samples and incubated at 50° C. for 30 minutes. The samples were then quickly placed on a magnet, and the supernatant was removed. The samples were washed twice with 50 mM EDTA at 50° C. for 3 minutes, then were removed quickly from the magnet. Samples were washed twice in Tween Wash Buffer for 2 minutes at 55° C., then were removed quickly from the magnet. The samples were washed with 10 mM Tris-HCl, pH8.0.

viii. PCR and Post-PCR Size Selection

The beads were resuspended in 50 μL of PCR master mix (use Nextera XT DNA library preparation kit from Illumina, #15028212 with dual-Index adapters #15055289). PCR was performed using the following program. The cycle number was estimated using one of two methods: (1) A first run of 5 cycles (72° C. for 5 minutes, 98° C. for 1 minute, 98° C. for 15 seconds, 63° C. for 30 seconds, 72° C. for 1 minute) is performed on a regular PCR and then the product is removed from the beads. Then, 0.25×SYBR green is added, and the sample is run on a qRT-PCR. Samples are pulled out at the beginning of exponential amplification; or (2) Reactions are run on a PCR and the cycle number is estimated based on the amount of material from the post-ChIP Qubit (greater than 50 ng is run in 5 cycles, while approximately 50 ng is run in 6 cycles, 25 ng is run in 7 cycles, 12.5 ng is run in 8 cycles, etc.).

Libraries were placed on a magnet and eluted into new tubes. The libraries were purified using a kit form Zymo Research and eluted into 10 μL of water. A two-sided size selection was performed with AMPure XP beads. After PCR, the libraries were placed on a magnet and eluted into new tubes. Then, 25 μL of AMPure XP beads were added, and the supernatant was kept to capture fragments less than 700 bp. The supernatant was transferred to a new tube, and 15 μL of fresh beads were added to capture fragments greater than 300 bp. A final elution was performed from the Ampure XP beads into 10 μL of water. The library quality was verified using a Bioanalyzer.

ix. Buffers

Hi-C Lysis Buffer (10 mL) contains 100 μL of 1M Tris-HCl pH 8.0; 20 μL of 5M NaCl; 200 μL of 10% NP-40; 200 μL of 50× protease inhibitors; and 9.68 mL of water. Nuclear Lysis Buffer (10 mL) contains 500 μL of 1M Tris-HCl pH 7.5; 200 μL of 0.5M EDTA; 1 mL of 10% SDS; 200 μL of 50× Protease Inhibitor; and 8.3 mL of water. ChIP Dilution Buffer (10 mL) contains 10 μL of 10% SDS; 1.1 mL of 10% Triton X-100; 24 μL of 500 mM EDTA; 167 μL of 1M Tris pH 7.5; 334 μL of 5M NaCl; and 8.365 mL of water. Low Salt Wash Buffer (10 mL) contains 100 μL of 10% SDS; 1 mL of 10% Triton X-100; 40 μL of 0.5M EDTA; 200 μL of 1M Tris-HCl pH 7.5; 300 μL of 5M NaCl; and 8.36 mL of water. High Salt Wash Buffer (10 mL) contains 100 μL of 10% SDS; 1 mL of 10% Triton X-100; 40 μL of 0.5M EDTA; 200 μL of 1M Tris-HCl pH 7.5; 1 mL of 5M NaCl; and 7.66 mL of water. LiCl Wash Buffer (10 mL) contains 100 μL of 1M Tris pH 7.5; 500 μL of 5M LiCl; 1 mL of 10% NP-40; 1 mL of 10% Na-deoxycholate; 20 μL of 0.5M EDTA; and 7.38 mL of water.

DNA Elution Buffer (5 mL) contains 250 μL of fresh 1M NaHCO₃; 500 μL of 10% SDS; and 4.25 mL of water. Tween Wash Buffer (50 mL) contains 250 μL of 1M Tris-HCl pH 7.5; 50 μL of 0.5M EDTA; 10 mL of 5M NaCl; 250 μL of 10% Tween-20; and 39.45 mL of water. 2× Biotin Binding Buffer (10 mL) contains 100 μL 1M Tris-HCl pH 7.5; 20 μL of 0.5M; 4 mL of 5M NaCl; and 5.88 mL of water. 2×TD Buffer (1 mL) contains 20 μL of 1M Tris-HCl pH 7.5; 10 μL of 1M MgCl₂; 200 μL of 100% Dimethylformamide; and 770 μL of water.

R. Determining the IC50

The optimal concentration prior to cell toxicity (IC50) was determined for each compound that mimics or rescues the DBA phenotype. First, the IC50 for 4 different compounds in a TF1 cell line and CB-CI-IRES-CD34+ primary cells were determined. Nutlin 3 is a potent and selective MDM2 (RING finger-dependent ubiquitin protein ligase for itself and p53) antagonist with an IC50 of 90 nM in a cell-free assay. It has also been observed to stabilize p73 in p53-deficient cells. Pifithrin-α is an inhibitor of p53, which inhibits p53-dependent transactivation of p53-responsive genes. Nutlin 3a, an active enantiomer of Nutlin 3, inhibits the p53/MDM2 interaction with an IC50 of 90 nM in a cell-free assay. Cyclic Pifithrin-a was also analyzed. Three 96-well plates were used for analysis of each cell type.

Three rows were dedicated to each compound, and column 12 contained cells+media/2% DMSO. Compound dilutions were made in 11 tubes containing media+2% DMSO (11×5=55×1.5 ml tubes per cell type). Two conical tubes of media+2% DMSO were prepared for each media type (RPM1-GMCSF or Diff media-dox). Seven ml of media was combined with each drug. Therefore, 35 ml was needed for each cell type, and 50 mL+2% DMSO media was made. The compounds were diluted to 10 mM in DMSO. Cells were seeded at 100K cells/well in 50 μL of media. Cells were diluted to 2M cells/ml. One hundred eight wells were needed for each cell type (72 wells on 2 plates, 36 wells on 1 plate).

For each drug concentration, 150 μL of each dilution was analyzed in triplicate for each cell type. 0.5 ml of each drug concentration was made resulting in 300 μL extra.

Drug concentrations analyzed were as shown in Table 26.

TABLE 26
Stock drug concentrations

| | | | |
|---|---|---|---|
| Tube | Conc. | Starting Vol. | End Vol. |

| | | | | | | |
|---|---|---|---|---|---|---|
| 1 | 200 | μM | 1 | mL | 0.5 | ml |
| 2 | 100 | μM | 0.5 | ml | 0.5 | ml |
| 3 | 50 | μM | 0.5 | ml | 0.5 | ml |
| 4 | 25 | μM | 0.5 | ml | 0.5 | ml |
| 5 | 12.5 | μM | 0.5 | ml | 0.5 | ml |
| 6 | 6.25 | μM | 0.5 | ml | 0.5 | ml |
| 7 | 3.125 | μM | 0.5 | ml l | 0.5 | ml |
| 8 | 1.5 | μM | 0.5 | ml | 0.5 | ml |
| 9 | 0.7 | μM | 0.5 | ml | 0.5 | ml |
| 10 | 0.35 | μM | 0.5 | ml | 0.5 | ml |
| 11 | 0.175 | μM | 0.5 | ml | 1 | ml |

| | | | |
|---|---|---|---|
| 12 | Control | Control | Control |

0.5 mL of DMSO-media was added to each of 551.5 ml tubes. Twenty μL of 10 mM stock and 480 μL of additional media was added into tube 1. 10.8 million cells were needed in 5.4 ml aliquots for 108 wells with 100,000 cells/well in 50 μL of liquid. Eight ml of cells at 2M/ml were collected for a total of 16 million cells. An additional 5.2 million cells were transferred by 2.6 ml aliquots to 2 sterile reservoirs (for 10 ml of cells). The final titration calculation was as follows: 8 ml of 2 million/cells=16 million cells/8 ml=2,000 cells/μL×50 μL=100,000 cells/50 μL/well.

S. Biological Activity of Maximal Compound Dosages

The biological activity of compounds inn TF1 and CI-CB-CD34+ cells was studied by choosing a maximal dose that does not cause cell toxicity.

The concentration of 10 μM was used for compounds Nutlin 3, TFP, Pifithrin alpha, Nutlin 3a, and cyclic Pifithrin alpha. The stock for each compound was at 10 mM and were diluted to 100 μM with RMPI+GCMSF (2% DMSO). Cells were collected and resuspended in 8 mL of RPMI+GMCSF at a concentration of 1×10⁶ cells/mL. For the TF1 cells, at each well of a 6-well plate were added 1 mL of cells, 300 μL of 100 μM compound dilution, and 1.7 mL of RMPI+GCMSF+1.5% DMSO (1% final DMSO concentration in 3 mL total). One mL of the CI-CB-CD34+ cells, 300 μL of 100 μM compound dilution, and 700 μL of RMPI+GCMSF (0.1% final DMSO concentration in 2 mL total) were added to each well of a 6-well plate.

T. Dose Titration of Compounds into RPS19 KD TF1 Cells

The concentration of the compounds needed to observe a rescued DBA phenotype in TF1 RPS19 KD cells was determined via qRT-PCR analysis of p53 levels. The concentrations of drugs used in TF1 cells above that did not result in cell toxicity were 3.125 μM (Dose 3), 6.25 μM (Dose 2), and 25 μM (Dose 1). Nine different cell lines were used. The wild type (WT) TF1 and 8 TF1 KD cell lines transduced with 1 of the following plasmid: A) pSMART PGK GFP shNT-dox inducible; B) pSMART PGK GFP shRSP19_1-dox inducible; C) pSMART PGK GFP shRPS19_2-dox inducible; D) pSMART EF1alpha GFP shNT-dox inducible; 1) pLKO RPS19_sh4-constitutive; J) pLKO RPS19_sh5-constitutive; K) pLKO shNT-constitutive; and L) PLKO RPS19_sh1-constitutive. Cells were collected in separate tubes in a concentration of 1000 cells/μL to reach a concentration of 50,000 cells/50 μL per well. Cell were centrifuged at 1,200 rpm for 5 minutes. Then, the media was aspirated and cells were resuspended in RPMI+GCMSF medium.

For the preparation of Dose 1 (final concentration on plate 25 μM), a 50 μM solution was made by adding 300 μL of 100 μL stock to 300 μL of RPM1+GCMSF+2% DMSO (final 1% DMSO). The 50 μM solution was further diluted to 2× when added with the cells into a well, resulting in a final concentration of 25 μM. For the preparation of Dose 2 (final concentration on plate 6.125 μM), a 12.25 μM solution was made by adding 75 μL of 100 μL stock to 525 μL of RPM1+GCMSF+2% DMSO. The 1225 μM solution was further diluted 2× when added with the cells into a well, resulting into a final concentration of 6.125 μM. For the preparation of Dose 3 (final concentration on plate 3.125 μM), a 6.25 μM solution was made by adding 37.5 μL of 100 μL stock to 562.5 μL of RPMI+GCMSF+2% DMSO. The 6.25 μM solution was further diluted 2× when added with the cells into a well, resulting in a final concentration of 3.125 μM. DMSO 1% was used for each cell type as DMSO control.

A 96-well plate was used for analysis of each dose on each cell type. Fifty μL of each cell type and 50 μL of a diluted compound are added to the corresponding wells for a total of 100 μL RPMI+GCMSF+1% DMSO, with 50,000 cells/well. The following day, 50 μL for RNA (25,000 cells) and 50 μL for viability ATP assay (25,000 cells) were collected from each well.

U. Drug Dilutions for Administration to Hepatocytes

Prior to compound treatment of hepatocytes, the drugs and bioactive compounds were diluted according to the parameters in Table 27. For the drugs, 100 mM stock drugs in DMSO were diluted to 10 mM by mixing 0.1 mM of the stock drug in DMSO with 0.9 ml of DMSO to a final volume of 1.0 ml. Five μl of the diluted drug was added to each well, and 0.5 ml of media was added per well of drug. Each drug was analyzed in triplicate. Dilution to 1000× was performed by adding 5 μl of drug into 45 μl of media, and the 50 μl being added to 450 μl of media on cells.

Table 27 provides the weight of the compound for a 100 mM dilution in 1 ml of DMSO, the volume of DMSO in the 100 mM dilution, the amount of added volume, the final concentration, and the storage temperature for the dry compound.

TABLE 27
Dilution values for drug compounds

| Compound | MW (Da) | Weight (mg) | DMSO (ml) | Added vol. (ml) | Final conc. (mM) | Storage | |
|---|---|---|---|---|---|---|---|
| Simvastatin | 418.6 | Net weighed | 1.2 | Not diluted | | 4° | C. |
| Adapin (dexepin) | 315.8 | 31.6 | | 1.0 | 95.7 | RT | |
| Danazol | 337.5 | 33.7 | | 1.0 | 100.0 | RT | |
| Rosiglitazene maleate | 473.5 | 47.4 | | 1.0 | 96.6 | −20° | C. |
| Sulpiride | 341.4 | 34.1 | | 1.1 | 89.6 | 4° | C. |
| Ranitidine | 350.9 | 35.1 | | 1.0 | 100.5 | RT | |
| Metformin | 165.6 | 16.6 | | 1.1 | 92.5 | −20° | C. |
| Imatinib | 589.7 | Not weighed | 1.7 | Not diluted | | 4° | C. |
| Papaverine | 375.9 | 37.6 | | 1.1 | 89.7 | RT | |
| Miodarone | 681.8 | 68.2 | | 1.1 | 91.5 | −20° | C. |
| Nitrofurantoin | 238.2 | 23.8 | | 1.3 | 78.6 | RT | |
| Prednisone | 358.4 | 35.8 | | 1.2 | 84.3 | RT | |
| Penicillamine(D-) | 149.2 | 14.9 | | | | | |
| Rifampicin | 822.9 | 82.3 | | 1.0 | 99.5 | −20° | C. |
| Benzbromarone | 424.1 | 42.4 | | 1.1 | 92.0 | 4° | C. |
| Isoniazid | 137.1 | 13.7 | | 1.5 | 66.9 | RT | |
| Acetaminophen (paracetamol) | 151.2 | 15.1 | | 1.0 | 100.0 | −20° | C. |
| Ritonavir | 720.9 | Not weighed | 1.4 | Not diluted | | RT | |
| SGI-1776 | 405.4 | Not weighed | 1.2 | Not diluted | | | |
| Valproate | 166.2 | 16.6 | | 1.3 | 79.9 | RT | |
| Ibuprofen | 206.3 | 20.6 | | 1.4 | 69.0 | RT | |
| Propylthiouracil | 170.2 | 17.0 | | 1.0 | 100.0 | −20° | C. |
| Rapamycin | 914.2 | Not weighed | 0.5 | Not diluted | | −20° | C. |

Bioactive compounds were also administered to hepatocytes. To obtain 1000× stock of the bioactive compounds in 1 ml DMSO, 0.1 ml of 10,000× stock was combined with 0.9 ml DMSO. Bioactive compounds as diluted had the characteristics shown in Table 28.

TABLE 28
Dilution values for bioactive compounds

| Compound | MW (Da) | Final conc. | | 1000x conc. | | DMSO (ml) | Drug (ml) | 1000x (mg) | 10000x (mg) |
|---|---|---|---|---|---|---|---|---|---|
| BIO | 356.2 | 2 | μM | 2 | mM | 0.7 | | 0.7 | 7.1 |
| ATRA | 300.4 | 2 | μM | 2 | mM | 8.3 | | 0.6 | 6.0 |
| xav939 | 312.3 | 1 | μM | 1 | mM | 1.6 | | 0.3 | 3.1 |
| bms777607 | 512.9 | 5 | μM | 5 | mM | 0.2 | | 2.6 | 25.6 |
| dmPGE2 | 380.5 | 1 | μM | 1 | mM | 0.3 | | 0.4 | 3.8 |
| MK-0752 | 442.9 | 20 | μM | 20 | mM | None | 0.6 (DMSO) | 8.9 | Not prepared |
| N-Acetylpurinomycin | 513.6 | 100 | ng/ml | 100 | μg/ml | 10.0 | | 0.1 | 1.0 |
| LY 364947 | 272.3 | 3 | μM | 3 | mM | 6.1 | | 0.8 | 8.2 |

V. Transduction of Hematopoietic Cells with Conditional Immortalization Transgenes

Cryopreserved CD34+ cells (cord blood, peripheral blood, or bone marrow derived) were thawed from cryovials (typically 1×10⁶ or 5×10⁶ cells/vial) at 37° C. until completely thawed. Cells were then grown at a density of 1×10⁶ cells/mL for a period of 11 days in Hematopoietic Stem Cell expansion media DXF (Promocell) for the expansion of hematopoietic progenitor cells. Expanded CD34+ progenitor cells were then grown for an additional 3 days in SFEMII medium (Stem Cell Technologies) supplemented with the Erythroid Expansion supplement (Stem Cell Technologies). Cells can be efficiently cryopreserved at this step and are ready for viral transduction.

The Lenti-X Tet-One Inducible Expression System (Puro) (Clontech, Cat. No. 631847) was used for the transfection of 293FT cells. pLVX-TetOne-Puro vector was modified to exchange the Puro cassette with a Blasticidin (BLA), and full length cMYC, BCL-XL, BCL-XL-T2A-MYC and BCL-XL-IRES-MYC were synthesized and cloned into the pLVX-TetOne-Puro and pLVX-TetOne-BLA vectors.

293FT cells (Clontech) were transfected with 7 μg of pLVX-TetOne-Puro lentiviral vector combined with Lenti-X Packaging Single Shots (VSV-G) plasmids (Clontech). 293FT cells were transduced using the plasmid mixture. 48 hours after the transduction, the viral supernatant was collected and filtered through a 0.45 μM PVDF unit. The viral particles were concentrated by adding ⅓ of the total volume of Lenti-X Concentrator (Clontech). The mixture was mixed well by inversion and incubated for 1-2 hours at 4° C. After precipitation, the tubes were centrifuged at 4° C. for more than 45 minutes at 1,500 g to pellet the virus. Then, the supernatant was aspirated and the pellet was resuspended in PBS (to a concentration of 25-50×). Plates were coated with retronectin and incubated for more than 4 hours at RT or overnight at 4° C., using the Retronectin-transduction protocol (Clontech) according to the manufacturer's instructions. Following the Retronectin-incubation protocol (Clontech), the plates were blocked with 2% BSA/PBS for more than 30 minutes, and washed with PBS. Target cells were seeded onto the Retronectin-coated plates and the appropriate amount of virus was added. Then the plates were spun at 2,500 rpm for 1-2 hours at room temperature. Cells were incubated at 37° C. for 72 hours following the infection, prior to selection.

For the selection, antibiotic was added in the cell culture medium (for example 5 μg/mL of PURO). Following the death of all the non-resistant cells, the surviving cells were collected for RNA and protein assays. For RNA extraction, the cells were harvested in 500 μL of TRIZOL reagent (Thermo Scientific), and incubated for 5 minutes at RT. Then, 100 μL of chloroform was added to the cells, and the mixture was vortexed and then centrifuged at 12,000 g for 15 minutes. The aqueous phase was transferred to fresh tubes and one volume of 70% ethanol was added. The TRIZOL Plus RNA purification Kit (Thermo Scientific) was used for the remaining extraction protocol according to the manufacturer's instructions. For the conversion of RNA to cDNA the High Capacity cDNA RT kit (Thermo Scientific) was used according to the manufacturer's instructions. The cDNA was analyzed with qRT-PCR to determine the fold change of knockdown cells using Taqman Fast PCR mix and Applied Biosystems probes (Thermo Scientific).

### Example 2. Gene Expression in Stimulated Hepatocytes

To identify genes modulated with small molecules, primary human hepatocytes were prepared as a monoculture, and 39 small molecules that are known cell signaling agonists/antagonists were applied to the cells. The 39 compounds chosen as perturbation stimuli were known in the art to modulate at least one canonical cellular pathway. These compounds are listed in Table 29 with their category, DrugBank identifier (DBO), pathway, gene target, and pharmaceutical action. It is noted that the unique identifiers from ENSEMBL for the “Overlapping Gene(s)” have been modified to remove the first five leading zeros (0) of the identifier after the ENSG label (ENSG00000).

TABLE 29
Pathways associated with 39 compounds

| Compound | Category | DB0 | Pathway | Target | ENSG | Action |
|---|---|---|---|---|---|---|
| Valproate | Drug | 0313 | GABAergic synapse | ABAT/HDAC9 | Fusion | inhibitor |
| Atenolol | Drug | 0335 | Adrenergic signaling | ADRB1 | 043591 | antagonist |
| Amiodarone | Drug | 1118 | Adrenergic signaling | ADRB1 | 043591 | antagonist |
| Dorsomorphin | Bioactive | | TGF beta signaling | ALK | 171094 | inhibitor |
| Imatinib | Drug | 0619 | Ras & ErbB signaling | BCR/ABL | Fusion | inhibitor |
| BMP2 | Bioactive | | TGF beta signaling | BMPR1A | 107779 | agonist |
| Penicillamine(D-) | Drug | 0859 | | Copper | NA | chelator |
| Benzbromarone | Drug | | Arachidonic acid metabolism | CYP2C9 | 138109 | inhibitor |
| Sulpiride | Drug | 0391 | cAMP signaling | DRD2 | 149295 | antagonist |
| Danazol | Drug | 1406 | Estrogen signaling | ESR1 | 091831 | agonist |
| Prednisone | Drug | 0635 | Neuroactive ligand-receptor interaction | GR | NA | agonist |
| BIO | Bioactive | 3444 | TGF beta signaling | GSK3B | 082701 | inhibitor |
| Simvastatin | Drug | 0641 | Cholesterol synthesis | HMGCR | 113161 | inhibitor |
| dmPGE2 | Bioactive | | Arachidonic acid metabolism | HPGD | 164120 | inhibitor |
| Ranitidine | Drug | 0863 | Calcium signaling | HRH2 | 113749 | antagonist |
| Adapin (doxepin) | Drug | 1142 | Calcium signaling | HTR2A | 102468 | antagonist |
| Nefazodone | Drug | 1149 | Calcium signaling | HTR2A | 102468 | antagonist |
| bms777607 | Bioactive | | Ras signaling | MET | 105976 | inhibitor |
| Captopril | Drug | 1197 | Estrogen signaling | MMP2 | 087245 | inhibitor |
| Rapamycin | Drug | 0877 | mTOR signaling | mTOR | 198793 | inhibitor |
| Papaverine | Drug | 1113 | cAMP signaling | PDE4B | 184588 | inhibitor |
| SGI-1776 | Drug | | Jak-STAT signaling | PIM1 | 137193 | inhibitor |
| Rifampicin | Drug | 1045 | Antibiotic | Pol | NA | inhibitor |
| ritonavir | Drug | 0503 | Transcription | Pol | NA | inhibitor |
| Rosiglitazone maleate | Drug | 0412 | PPAR signaling | PPARG | 132170 | agonist |
| Metformin | Drug | 0331 | Insulin & AMPK signaling | PRKAB1 | 111725 | agonist |
| Acetaminophen | Drug | 0316 | Arachidonic acid metabolism | PTGS1 | 095303 | inhibitor |
| ibuprofen | Drug | 1050 | NF-κB signaling | PTGS2 | 073756 | inhibitor |
| bms189453 | Bioactive | | Nuclear Receptor transcription | RARB | 077092 | agonist |
| ATRA | Bioactive | 0755 | Nuclear Receptor transcription | RXRB | 204231 | agonist |
| Disopyramide | Drug | 0280 | Adrenergic signaling | SCN5A | 183873 | inhibitor |
| N-Acetylpurinomycin | Bioactive | | TGF beta signaling | SKI and SKIL | 136603 | modulator |
| bms833923 | Bioactive | | Hedgehog signaling | SMO | 128602 | antagonist |
| LY 364947 | Bioactive | | TGF beta signaling | TGFBR1 | 106799 | inhibitor |
| xav939 | Bioactive | | WNT & PARP inhibitor | TNK1 | 174292 | inhibitor |
| Propylthiouracil | Drug | 0550 | Thyroid hormone synthesis | TPO | 115705 | inhibitor |
| Nitrofurantoin | Drug | 0698 | Antibiotic | ydbK | NA | potentiator |
| MK-0752 | Bioactive | | Alzheimer's disease | γ-secretase | NA | inhibitor |
| Isoniazid | Drug | 0951 | Arachidonic acid metabolism | NA | NA | NA |

Table 30 summarizes the known diseases and genes that are considered to be modulated by each of the 39 small molecules.

TABLE 30
Disease associated genes and modulating compounds

| Gene | Disease | Compound |
|---|---|---|
| COL1A1 | CAFFEY DISEASE | BIO, nitrofurantoin, rifampicin |
| SLC37A4 | GLYCOGEN STORAGE DISEASE Ib; GSD1B | Prednisone |
| SLC2A2 | FANCONI-BICKEL SYNDROME; FBS | Rifampicin, MK-0752, Prednisone |
| AASS | HYPERLYSINEMIA, TYPE I | Rosiglitazone |
| ABCB11 | CHOLESTASIS, PROGRESSIVE FAMILIAL | Prednisone, Ritonavir, Rapamycin |
| | INTRAHEPATIC, 2; PFIC2 | |
| ABCC11 | METABOLIC | Prednisone, Rifampicin, Benzbromarone, |
| | | MK-0752, Rosiglitazone |
| ABCG2 | BLOOD GROUP, JUNIOR SYSTEM; JR | BIO |
| ACACB | METABOLIC | BIO |
| ACADVL | ACYL-CoA DEHYDROGENASE, VERY LONG-CHAIN, | Rosiglitazone |
| | DEFICIENCY OF; ACADVLD | |
| ACAT2 | METABOLIC | Simvastatin |
| ACSL1 | METABOLIC | Rosiglitazone |
| ACSL5 | METABOLIC | Rosiglitazone |
| ACTG2 | VISCERAL MYOPATHY; VSCM | Rapamycin, BIO, ATRA |
| ADAMTSL2 | GELEOPHYSIC DYSPLASIA 1; GPHYSD1 | Prednisone |
| AGTR1 | METABOLIC | Rifampicin |
| AKR1D1 | BILE ACID SYNTHESIS DEFECT, CONGENITAL, | Rapamycin |
| | 2; CBAS2 | |
| ALAS1 | METABOLIC | Rifampicin, Ritonavir, MK-0752 |
| AMACR | BILE ACID SYNTHESIS DEFECT, CONGENITAL, | Prednisone, Benzbromarone |
| | 4; CBAS4 | |
| ANGPTL2 | METABOLIC | Nitrofurantoin, Prednisone |
| AQP7 | METABOLIC | Rosiglitazone |
| ARG1 | ARGININEMIA | Prednisone |
| ASPA | METABOLIC | Rosiglitazone |
| BCO1 | HYPERCAROTENEMIA AND VITAMIN A DEFICIENCY, | Acetaminophen (paracetamol) |
| | AUTOSOMAL DOMINANT | |
| C10orf11 | METABOLIC | BIO |
| CA12 | METABOLIC | Rifampicin, ritonavir |
| CA2 | METABOLIC | Prednisone |
| CDKN1C | INTRAUTERINE GROWTH RETARDATION, | Imatinib, bms777607 |
| | METAPHYSEAL DYSPLASIA, ADRENAL HYPOPLASIA | |
| | CONGENITA, AND GENITAL ANOMALIES | |
| CECR1 | POLYARTERITIS NODOSA, | Nitrofurantoin, Benzbromarone |
| | CHILDHOOD-ONSET; PAN | |
| CIDEC | LIPODYSTROPHY, FAMILIAL PARTIAL, | Rosiglitazone, Nitrofurantoin, Prednisone |
| | TYPE 5; FPLD5 | |
| CLMP | CONGENITAL SHORT BOWEL SYNDROME; CSBS | Metformin, Imatinib, Nitrofurantoin, Simvastatin, |
| | | Isoniazid, Ibuprofen, Propylthiouracil, |
| | | bms777607, dmPGE2, LY 364947, Danazol |
| COL1A1 | CAFFEY DISEASE | BIO |
| COL3A1 | EHLERS-DANLOS SYNDROME, TYPE IV, | Penicillamine(D-), Isoniazid, Ritonavir, Valproate, |
| | AUTOSOMAL DOMINANT | Rapamycin, BIO, Rosiglitazone |
| CPS1 | CARBAMOYL PHOSPHATE SYNTHETASE I | Prednisone |
| | DEFICIENCY, HYPERAMMONEMIA DUE TO | |
| CPT1A | CARNITINE PALMITOYLTRANSFERASE I | Rosiglitazone |
| | DEFICIENCY | |
| CPT2 | CARNITINE PALMITOYLTRANSFERASE II | Rosiglitazone |
| | DEFICIENCY, LATE-ONSET | |
| CYBB | GRANULOMATOUS DISEASE, CHRONIC, | Adapin, Danazol, Penicillamine(D-), Rifampicin, |
| | X-LINKED; CGD | Acetaminophen, xav939 |
| CYP17A1 | METABOLIC | BIO |
| CYP21A2 | ADRENAL HYPERPLASIA, CONGENITAL, | Prednisone |
| | DUE TO isoniazid-HYDROXYLASE DEFICIENCY | |
| CYP27B1 | VITAMIN D HYDROXYLATION-DEFICIENT | Ranitidine, Nitrofurantoin |
| | RICKETS, TYPE 1A; VDDR1A | |
| CYP2C19 | DRUG METABOLISM, POOR, | Rifampicin, MK-0752 |
| | CYP2CRifampicin-RELATED | |
| CYP3A4 | METABOLIC | Prednisone, Rifampicin, Ritonavir, MK-0752, |
| | | Rosiglitazone |
| CYP7A1 | METABOLIC | Rosiglitazone, Imatinib, Prednisone, Rifampicin, |
| | | Simvastatin, Benzbromarone, Ritonavir, Rapamycin, |
| | | ATRA, bms777607, MK-0752 |
| DNAH5 | CILIARY DYSKINESIA, PRIMARY, 3; CILD3 | Prednisone |
| ECH1 | METABOLIC | Rosiglitazone |
| ENG | TELANGIECTASIA, HEREDITARY | Prednisone |
| | HEMORRHAGIC, OF RENDU, | |
| | OSLER, AND WEBER; HHT | |
| ENO3 | METABOLIC | Prednisone, Rifampicin, Ritonavir |
| ENPP3 | METABOLIC | Ritonavir, SGI-1776, N-Acetylpurinomycin |
| ETFDH | METABOLIC | Rosiglitazone |
| FAM111B | POIKILODERMA, HEREDITARY FIBROSING, | Metformin, Rapamycin |
| | WITH TENDON CONTRACTURES, MYOPATHY, | |
| | AND PULMONARY FIBROSIS; POIKTMP | |
| FERMT3 | LEUKOCYTE ADHESION DEFICIENCY, TYPE III; LAD3 | Isoniazid, Valproate |
| FGF19 | METABOLIC | Rifampicin, Ritonavir, ATRA, MK-0752 |
| G6PC | GLYCOGEN STORAGE DISEASE Ia; GSD1A | Prednisone, Rapamycin |
| GALE | GALACTOSE EPIMERASE DEFICIENCY | Ritonavir |
| GHR | LARON SYNDROME | MK-0752 |
| GLA | FABRY DISEASE | Ritonavir |
| GLIS3 | DIABETES MELLITUS, NEONATAL, WITH | Prednisone |
| | CONGENITAL HYPOTHYROIDISM | |
| GYS2 | GLYCOGEN STORAGE DISEASE 0, LIVER; GSD0A | Prednisone |
| HAL | METABOLIC | Prednisone |
| HGD | ALKAPTONURIA; AKU | Prednisone |
| HMGCR | METABOLIC | Simvastatin |
| HMGCS2 | 3-HYDROXY-3-METHYLGLUTARYL-CoASYNTHASE- | Rosiglitazone |
| | 2 DEFICIENCY; HMGCS2D | |
| HMOX1 | HEME OXYGENASE 1 DEFICIENCY; HMOX1D | Nitrofurantoin |
| HOGA1 | METABOLIC | Prednisone |
| HPGD | DIGITAL CLUBBING, ISOLATED CONGENITAL | Prednisone |
| ID1 | METABOLIC | bms777607, MK-0752, imatinib, Nitrofurantoin, |
| IFT80 | SHORT-RIB THORACIC DYSPLASIA2 WITH | Prednisone, Ritonavir |
| | OR WITHOUT POLYDACTYLY; SRTD2 | |
| IGFALS | METABOLIC | Ritonavir |
| LDHA | METABOLIC | Nitrofurantoin |
| LPA | METABOLIC | Rifampicin |
| LPIN2 | MAJEED SYNDROME | Prednisone |
| LRP2 | METABOLIC | Prednisone |
| LRP4 | METABOLIC | Imatinib, Benzbromarone |
| MOGAT2 | METABOLIC | Ritonavir |
| NAGS | N-ACETYLGLUTAMATE SYNTHASE | Prednisone |
| | DEFICIENCY; NAGSD | |
| NCF2 | GRANULOMATOUS DISEASE, CHRONIC, | Prednisone |
| | AUTOSOMAL RECESSIVE, | |
| | CYTOCHROME b-POSITIVE, TYPE II | |
| NOS3 | PREECLAMPSIA/ECLAMPSIA 1; PEE1 | Ranitidine, sulpiride |
| NPHP1 | NEPHRONOPHTHISIS1; NPHP1 | SGI-1776 |
| PCSK9 | METABOLIC | Simvastatin |
| PKHD1 | POLYCYSTIC KIDNEY DISEASE, AUTOSOMAL | Prednisone |
| | RECESSIVE; ARPKD | |
| PKLR | PYRUVATE KINASE DEFICIENCY OF RED CELLS | Benzbromarone, Ritonavir |
| PLA2G6 | METABOLIC | Rinonavir, Rapamycin |
| POR | ANTLEY-BIXLER SYNDROME WITH GENITAL | Prednisone, Rifampicin, Ritonavir, MK-0752, |
| | ANOMALIES AND DISORDERED STEROIDOGENESIS; | Rosiglitazone |
| | ABS1 | |
| PRODH | HYPERPROLINEMIA, TYPE I; HYRPRO1 | Prednisone |
| RMRP | CARTILAGE-HAIR HYPOPLASIA; CHH | Imatinib, Papaverine, Amiodarone, Prednisone, |
| | | Penicillamine (D-), Rifampicin, Benzbromarone, |
| | | Isoniazid, Ibuprofen |
| ROR2 | ROBINOW SYNDROME, AUTOSOMAL | Nitrofurantoin |
| | RECESSIVE; RRS | |
| SLC22A4 | METABOLIC | Rifampicin |
| SLC25A20 | CARNITINE-ACYLCARNITINE TRANSLOCASE | Rosiglitazone |
| | DEFICIENCY; CACTD | |
| SLC29A4 | METABOLIC | Nitrofurantoin |
| SLC2A2 | FANCONI-BICKEL SYNDROME; FBS | Rifampicin, MK-0752 |
| SLC36A2 | METABOLIC | Ibuprofen |
| SLC4A5 | METABOLIC | Rapamycin |
| SLC51B | METABOLIC | Rifampicin |
| SLC6A17 | METABOLIC | Imatinib, Prednisone, Isoniazid, Acetaminephen, |
| | | Rapamycin, ATRA, N-Acetylpurinomycin |
| SLC6A20 | METABOLIC | Rifampicin |
| SLC6A6 | METABOLIC | Rifampicin, BIO |
| SLCO4C1 | METABOLIC | Simvastatin |
| SOAT2 | METABOLIC | Ritonavir, ATRA |
| SORT1 | LOW DENSITY LIPOPROTEIN CHOLESTEROL | Prednisone, Benzbromarone, MK-0752 |
| | LEVEL QUANTITATIVE TRAIT LOCUS; LDLCQ | |
| SPINT2 | DIARRHEA 3, SECRETORY SODIUM, | Prednisone |
| | CONGENITAL, WITH OR WITHOUT OTHER | |
| | CONGENITAL ANOMALIES; DIAR3 | |
| SULT2A1 | METABOLIC | Rosiglitazone |
| TAT | TYROSINEMIA, TYPE II; TYRSN2 | Prednisone |
| TDO2 | METABOLIC | Prednisone |
| TMEM135 | METABOLIC | Rosiglitazone |
| TNFSF11 | OSTEOPETROSIS, AUTOSOMAL | Simvastatin |
| | RECESSIVE 2; OPTB2 | |
| TTPA | VITAMIN E, FAMILIAL ISOLATED | Rosiglitazone |
| | DEFICIENCY OF; VED | |
| VLDLR | METABOLIC | Rosiglitazone, Rifampicin, Ritonavir |
| WAS | WISKOTT-ALDRICH SYNDROME; WAS | xav939, N-Acetylpurinomycin |

A. Pilot RNA-Seq Results

To identify genomic signaling centers that were altered by administering the perturbation stimuli, changes in expression of 27,011 genes were measured by RNA-seq after administration of the 39 small molecule compounds in Table 29. Changes in expression having a p-value≤0.05 were considered significant. Table 31 provides the reference numbers used to identify these compounds herein.

TABLE 31
Compound names and reference numbers

| Name | MT | Name | MT | Name | MT |
|---|---|---|---|---|---|
| Simvastatin | MT-51 | Acetaminophen (paracetamol) | MT-922 | Bms777607 | MT-375 |
| Adapin (doxepin) | MT-723 | Ritonavir | MT-372 | Bms833923 | MT-636 |
| Danazol | MT-487 | SGI-1776 inhibitor | MT-254 | DmPGE2 | MT-937 |
| Nefazodone | MT-505 | Valproate | MT-625 | MK-0752 inhibitor | MT-308 |
| Rosiglitazone maleate | MT-361 | Ibuprofen | MT-662 | N-Acetylpurinomycin | MT-993 |
| Sulpiride | MT-277 | Propylthiouracil | MT-127 | LY 364947 inhibitor | MT-740 |
| Captopril | MT-835 | Rapamycin | MT-882 | Nitrofurantoin | MT-152 |
| Atenolol | MT-193 | BIO inhibitor | MT-209 | Prednisone | MT-861 |
| Ranitidine | MT-610 | Tretinoin (ATRA) | MT-603 | Penicillamine(D-) | MT-741 |
| Metformin | MT-117 | Xav939 | MT-713 | Disopyramide | MT-189 |
| Imatinib | MT-182 | Bms189453 agonist | MT-232 | Rifampicin | MT-901 |
| Papaverine | MT-913 | Dorsomorphin | MT-334 | Benzbromarone | MT-220 |
| Amiodarone | MT-641 | BMP2 | MT-543 | Isoniazid | MT-312 |

Fold change was calculated by dividing the level of expression in the cell system that had been perturbed by the level of expression in an unperturbed system. Expression of 2116 genes was observed to be significantly modulated by at least one of the compounds.

According to these results, thirty-one of the small molecules were observed to be effective in causing an at least 2-fold expression change in expression of at least one of the 105 disease associated genes (DAGs) of the 2,116 genes. Simvastatin, rosiglitazone, imatinib, nitrofurantoin, prednisone, rifampicin, benzbromarone, ritonovir, rapamycin, BIO, ATRA, MK-0752 were observed to be responsible for at least a 2-fold change in expression for most of the 105 disease associated genes. These genes and associated disease(s) are shown in Table 32.

TABLE 32
Modulated disease associated genes

| Gene | Disease |
|---|---|
| AASS | HYPERLYSINEMIA, TYPE I |
| ABCB11 | CHOLESTASIS, PROGRESSIVE FAMILIAL |
| | INTRAHEPATIC, TYPE 2; PFIC2 |
| ABCC11 | METABOLIC |
| ABCG2 | BLOOD GROUP, JUNIOR SYSTEM; JR |
| ACACB | METABOLIC |
| ACADVL | DEFICIENCY OF ACYL-CoA DEHYDROGENASE, |
| | VERY LONG-CHAIN; ACADVLD |
| ACAT2 | METABOLIC |
| ACSL1 | METABOLIC |
| ACSL5 | METABOLIC |
| ACTG2 | VISCERAL MYOPATHY; VSCM |
| ADAMTSL2 | GELEOPHYSIC DYSPLASIA 1; GPHYSD1 |
| AGTR1 | METABOLIC |
| AKR1D1 | BILE ACID SYNTHESIS DEFECT, CONGENITAL, |
| | TYPE 2; CBAS2 |
| ALAS1 | METABOLIC |
| AMACR | BILE ACID SYNTHESIS DEFECT, CONGENITAL, |
| | 4; CBAS4 |
| ANGPTL2 | METABOLIC |
| AQP7 | METABOLIC |
| ARG1 | ARGININEMIA |
| ASPA | METABOLIC |
| BCO1 | HYPERCAROTENEMIA AND VITAMIN A |
| | DEFICIENCY, AUTOSOMAL DOMINANT |
| C10orf11 | METABOLIC |
| CA12 | METABOLIC |
| CA2 | METABOLIC |
| CDKN1C | INTRAUTERINE GROWTH RETARDATION, |
| | METAPHYSEAL DYSPLASIA, ADRENAL |
| | HYPOPLASIA CONGENITA, AND GENITAL |
| | ANOMALIES |
| CECR1 | POLYARTERITIS NODOSA, CHILDHOOD-ONSET; |
| | PAN |
| CIDEC | LIPODYSTROPHY, FAMILIAL PARTIAL, TYPE 5; |
| | FPLD5 |
| CLMP | CONGENITAL SHORT BOWEL SYNDROME; CSBS |
| COL1A1 | CAFFEY DISEASE |
| COL3A1 | EHLERS-DANLOS SYNDROME, TYPE IV, |
| | AUTOSOMAL DOMINANT |
| CPS1 | CARBAMOYL PHOSPHATE SYNTHETASE I |
| | DEFICIENCY, HYPERAMMONEMIA DUE TO |
| CPT1A | CARNITINE PALMITOYLTRANSFERASE I |
| | DEFICIENCY |
| CPT2 | CARNITINE PALMITOYLTRANSFERASE II |
| | DEFICIENCY, LATE-ONSET |
| CYBB | GRANULOMATOUS DISEASE, CHRONIC, X- |
| | LINKED; CGD |
| CYP17A1 | METABOLIC |
| CYP21A2 | ADRENAL HYPERPLASIA, CONGENITAL, |
| | DUE TO 21-HYDROXYLASE DEFICIENCY |
| CYP27B1 | VITAMIN D HYDROXYLATION-DEFICIENT |
| | RICKETS, TYPE 1A; VDDR1A |
| CYP2C19 | DRUG METABOLISM, POOR, CYP2C19-RELATED |
| CYP3A4 | METABOLIC |
| CYP7A1 | METABOLIC |
| DNAH5 | CILIARY DYSKINESIA, PRIMARY, 3; CILD3 |
| ECH1 | METABOLIC |
| ENG | TELANGIECTASIA, HEREDITARY HEMORRHAGIC, |
| | OF RENDU, OSLER, AND WEBER; HHT |
| ENO3 | METABOLIC |
| ENPP3 | METABOLIC |
| ETFDH | METABOLIC |
| FAM111B | POIKILODERMA, HEREDITARY FIBROSING, |
| | WITH TENDON CONTRACTURES, MYOPATHY, |
| | AND PULMONARY FIBROSIS; POIKTMP |
| FERMT3 | LEUKOCYTE ADHESION DEFICIENCY, |
| | TYPE III; LAD3 |
| FGF19 | METABOLIC |
| G6PC | GLYCOGEN STORAGE DISEASE Ia; GSD1A |
| GALE | GALACTOSE EPIMERASE DEFICIENCY |
| GHR | LARON SYNDROME |
| GLA | FABRY DISEASE |
| GLIS3 | DIABETES MELLITUS, NEONATAL, WITH |
| | CONGENITAL HYPOTHYROIDISM |
| GYS2 | GLYCOGEN STORAGE DISEASE 0; LIVER; |
| | GSD0A |
| HAL | METABOLIC |
| HGD | ALKAPTONURIA; AKU |
| HMGCR | METABOLIC |
| HMGCS2 | 3-HYDROXY-3-METHYLGLUTARYL-CoA |
| | SYNTHASE-2 DEFICIENCY; HMGCS2D |
| HMOX1 | HEME OXYGENASE 1 DEFICIENCY; HMOX1D |
| HOGA1 | METABOLIC |
| HPGD | DIGITAL CLUBBING, ISOLATED CONGENITAL |
| ID1 | METABOLIC |
| IFT80 | SHORT-RIB THORACIC DYSPLASIA 2 |
| | WITH OR WITHOUT POLYDACTYLY; SRTD2 |
| IGFALS | METABOLIC |
| LDHA | METABOLIC |
| LPA | METABOLIC |
| LPIN2 | MAJEED SYNDROME |
| LRP2 | METABOLIC |
| LRP4 | METABOLIC |
| MOGAT2 | METABOLIC |
| NAGS | N-ACETYLGLUTAMATE SYNTHASE |
| | DEFICIENCY; NAGSD |
| NCF2 | GRANULOMATOUS DISEASE, CHRONIC, |
| | AUTOSOMAL RECESSIVE, CYTOCHROME |
| | b-POSITIVE, TYPE II |
| NOS3 | PREECLAMPSIA/ECLAMPSIA 1; PEE1 |
| NPHP1 | NEPHRONOPHTHISIS 1; NPHP1 |
| PCSK9 | METABOLIC |
| PKHD1 | POLYCYSTIC KIDNEY DISEASE, |
| | AUTOSOMAL RECESSIVE; ARPKD |
| PKLR | PYRUVATE KINASE DEFICIENCY OF |
| | RED CELLS |
| PLA2G6 | METABOLIC |
| POR | ANTLEY-BIXLER SYNDROME WITH |
| | GENITAL ANOMALIES AND DISORDERED |
| | STEROIDOGENESIS; ABS1 |
| PRODH | HYPERPROLINEMIA, TYPE I; HYRPRO1 |
| RMRP | CARTILAGE-HAIR HYPOPLASIA; CHH |
| ROR2 | ROBINOW SYNDROME, AUTOSOMAL |
| | RECESSIVE; RRS |
| SLC22A4 | METABOLIC |
| SLC25A20 | CARNITINE-ACYLCARNITINE |
| | TRANSLOCASE DEFICIENCY; CACTD |
| SLC29A4 | METABOLIC |
| SLC2A2 | FANCONI-BICKEL SYNDROME; FBS |
| SLC36A2 | METABOLIC |
| SLC37A4 | GLYCOGEN STORAGE DISEASE Ib; GSD1B |
| SLC4A5 | METABOLIC |
| SLC51B | METABOLIC |
| SLC6A17 | METABOLIC |
| SLC6A20 | METABOLIC |
| SLC6A6 | METABOLIC |
| SLCO4C1 | METABOLIC |
| SOAT2 | METABOLIC |
| SORT1 | LOW DENSITY LIPOPROTEIN |
| | CHOLESTEROL LEVEL QUANTITATIVE |
| | TRAIT LOCUS 6; LDLCQ6 |
| SPINT2 | DIARRHEA 3, SECRETORY SODIUM, |
| | CONGENITAL, WITH |
| | OR WITHOUT OTHER CONGENITAL |
| | ANOMALIES; DIAR3 |
| SULT2A1 | METABOLIC |
| TAT | TYROSINEMIA, TYPE II; |
| | TYRSN2 |
| TDO2 | METABOLIC |
| TMEM135 | METABOLIC |
| TNFSF11 | OSTEOPETROSIS, AUTOSOMAL |
| | RECESSIVE 2; OPTB2 |
| TTPA | VITAMIN E, FAMILIAL ISOLATED |
| | DEFICIENCY OF; VED |
| VLDLR | METABOLIC |
| WAS | WISKOTT-ALDRICH SYNDROME; WAS |

Ranitidine was observed to significantly (p-value≤0.05) modulate ACAD11, ACTA1, ADGRG6, ADM, AHSG, ALPK2, AMACR, ANGPTL4, APOA4, BNIP3, C11orf96, CAPN13, CHST9, COL4A1, CPS1, CSE1L-AS1, CSRNP3, CYP1A1, CYP1A2, CYP26A1, EFNA1, ENO2, ENO3, EPPK1, F13B, FABP3, FAM69C, FBXO41, FLNA, GAL3ST1, GBAP1, GDF15, GHR, GNAO1, GPER1, HAVCR1, HK2, HMOX1, ID2, IFT80, IGF1, IGFBP1, INSIG1, ITGA7, ITGB6, KCNK5, KRT80, LAMC2, LDHA, LINC00102, LINC00862, LINC00881, LOC100507195, LOC101928401, LOXL4, LRRIQ3, MGAM, MICALCL, MT1E, MT1HL1, MT1X, MYOF, NDRG1, NDUFA4L2, NEXN, NFASC, P4HA1, PAPLN, PBX1, PCK1, PCSK9, PEG10, PFKFB4, PID1, PLIN4, PPP1R27, PROX1, RASD1, RGCC, RNF122, SCN8A, SLC16A7, SLC2A2, SNORD12, SPAG4, SPNS2, TCAF2, TCP10L, TFRC, TGFB2, THBS1, TMCC1, TMEM45A, TNC, TTBK1, UBALD2, and VWCE.

Metformin was observed to significantly (p-value≤0.05) modulate AHSG, AKAP12, ALAS1, ALPK2, APOA4, ARG1, BNIP3, CCDC152, CES5A, CHST9, CLDN4, CLVS1, CPS1, CXCL11, DCLK1, EFNA3, ELOVL2, ENO2, ENO3, EPO, ETNPPL, F13B, FADS1, FAM13A, FAM69C, FCAMR, FDFT1, FER1L4, FGF2, GADD45B, GDF15, GHR, HES4, HMGCR, HMGCS1, HSPB8, IFT80, IGFBP1, IGLL5, IL18, IL23A, ILDR2, INSIG1, ISM1, ITGB6, JAKMIP3, KCP, KRT7, LAMC2, LAPTM5, LDHA, LOC101929448, LOXL4, LRRIQ3, MATN2, MCM7, MIR210HG, MMP12, MOG, MSMO1, MT1E, MT1HL1, MT1X, MVD, MYOF, NAMPT, NCF2, NDUFA4L2, OXTR, PALMD, PDK1, PEG10, PEX5L, PID1, PLIN4, PRKRIP1, PROX1, PRSS23, RMI1, RMRP, SCGN, SLC2A2, SLC7A5, SNORD49A, SNX22, SORBS1, SPAG4, SPNS2, SPP1, SULT2A1, TCP10L, TDO2, TFRC, TGFB2, THBS1, TLR10, TMEM45A, TNC, TTPA, TUBA4A, UGT2B4, VCAN, and VWCE.

Imatinib was observed to significantly (p-value≤0.05) modulate expression of A1BG-AS1, ABALON, ABCA10, ABCA7, ABCC11, ABHD2, ABLIM3, ACAA2, ACACB, ACAD11, ACADVL, ACKR2, ACSL1, ACTA1, ACTA2, ADCY5, ADGRV1, ADM, AHRR, AHSG, AKAP12, AKR1B15, AKR1D1, ALAS1, ALDH8A1, ALPK2, AMACR, ANGPTL4, ANGPTL7, ANGPTL8, ANKRD23, ANKRD37, ANO1, ANXA13, APOA1-AS, AQP4, ARG1, ARHGEF26, ARHGEF26-AS1, ARL14, ARL4C, ASB14, ATOH8, ATP6V1C2, B3GNT5, B4GALNT1, BAG3, BATF, BCL2, BMF, BMP2, BRF2, BSPRY, C10orf11, C10orf35, C12orf50, C1orf116, C2CD4A, C2orf82, C4orf19, C5orf45, C9orf173-AS1, C9orf72, CA12, CA2, CACYBP, CADPS2, CAMK2G, CASKIN1, CBLB, CCDC152, CCDC85B, CCL2, CCL20, CCNE2, CD274, CDC6, CDKN1C, CEBPD, CELSR2, CHST9, CLDN2, CNN1, COL10A1, COL1A1, CPA4, CPT1A, CREB3L3, CREB5, CRIP3, CRY2, CRYM, CTAGE8, CUX2, CXCL10, CXCL11, CXCL8, CYP1A1, CYP1A2, CYP26A1, CYP2C19, CYP2C9, CYP3A4, CYP3A5, CYP7A1, CYP8B1, CYTH1, DCX, DDIT4L, DEDD2, DEFB132, DGKG, DNAH5, DNAJA4, DNAJB1, DNAJB4, DNM1, DSG2-AS1, ECM2, EDARADD, EFNA1, EFNA3, ELOVL2, ENO2, EPAS1, ERRFI1, ETNPPL, EVA1A, EVPLL, FA2H, FADS1, FAM134B, FAM13A, FAM219A, FAM222A, FAM47E, FAM49A, FAM69C, FAM83D, FAXC, FBXO30, FBXO41, FER1L4, FGF2, FKBP5, FLNA, FLRT3, FLVCR2, FMO1, FOS, FOSB, FOSL1, FSIP1, FSTL3, G6PC, GADD45B, GALE, GAREM1, GCNT2, GEM, GHR, GIPR, GLA, GPC6, GREM1, GREM2, HAL, HAVCR1, HGD, HHAT, HIVEP2, HKDC1, HMCN1, HMOX1, HOGA1, HSP90AA1, HSPA12A, HSPA1A, HSPA1B, HSPA1L, HSPA4L, HSPA5, HSPA6, HSPB1, HSPH1, ID1, ID2, IDO2, IFT80, IGF1, IGFALS, IGFBP1, IGFBP3, IGFBP5, IGLL5, IL18, IL22RA1, IL6R, IRF7, IRS2, ITGA3, ITGA5, ITIH4-AS1, JDP2, KANK1, KCNJ11, KCNJ8, KCNK5, KDELR3, KDM3A, KIAA0319, KIAA1462, KIF14, KLF5, KLF9, KLHDC7B, KLHL31, KLK4, KRT7, KRT80, KRT81, KSR2, LAMB1, LAMB3, LEFTY1, LGALS4, LHFPL5, LIF, LINC00261, LINC00313, LINC00504, LINC00856, LINC01016, LINC01018, LINC01057, LINC01314, LINC01554, LMCD1, LOC100128494, LOC100129931, LOC100133286, LOC100507195, LOC101929427, LOC101929516, LOC102724050, LOC102724153, LOC104968399, LOC105376575, LOC153910, LOC283585, LOC344887, LPAL2, LPIN2, LRP4, LRP8, LTBP1, MAFB, MAFF, MANF, MAP1B, MAP3K8, MATN2, MED31, MGAM, MIR1282, MIR210HG, MIR6087, MIR6723, MLLT11, MMP24, MMP3, MOGAT2, MSC, MST1R, MT1E, MT1HL1, MT1X, MTRNR2L4, MXD3, MYCL, MYH3, MYL9, MYLIP, MYOF, MYRIP, NAMPT, NAP1L2, NBPF20, NCKAP5, NDRG1, NDRG4, NDUFA4L2, NFASC, NLGN2, NLRP6, NPB, NPPB, NPR3, NPTX2, NPTXR, NR1D1, NR6A1, NRAP, NRBP2, NRG4, NUAK2, NUCB1-AS1, NUCB2, NUDT18, NUGGC, NXN, NYAP1, OLFM2, OSGIN1, OTUD3, OXTR, P4HA1, PABPC3, PALMD, PAPLN, PARK2, PBX1, PCBP2-OT1, PCK1, PCSK9, PDE2A, PDE4D, PDIA4, PDK4, PELI3, PER1, PFKFB3, PFKFB4, PFN1P2, PGM2, PHLDA1, PIGR, PITRM1-AS1, PLA2G6, PLIN2, PLK2, PLXDC1, PLXNA4, PMAIP1, PNLIPRP3, POR, POTEE, POU5F1, PPARGC1A, PPFIA4, PPP1R3C, PPP1R3G, PREX1, PRG4, PROM1, PRR15, PRSS23, PSORS1C3, RAB27B, RARB, RASD1, RASD2, RCL1, RGCC, RGS2, RHCG, RHOB, RHOF, RLF, RMI1, RMRP, RND1, RND3, RNF122, RNVU1-19, RORA, ROS1, RPL17, RPL23P8, RPPH1, RSAD2, RTP3, SALL1, SCGN, SCPEP1, SEC14L2, SEC14L4, SERPINB9, SESN3, SFN, SH3GLB2, SIPA1L2, SIX4, SLC16A3, SLC16A7, SLC17A1, SLC17A2, SLC18A2, SLC22A25, SLC2A10, SLC2A2, SLC30A2, SLC38A2, SLC4A8, SLC6A1, SLC7A5, SLCO2B1, SLITRK3, SMIM3, SMOC1, SNAI1, SNAI2, SNHG9, SNORD17, SNX22, SNX29, SOCS2, SOCS2-AS1, SOGA3, SORBS1, SORBS2, SPAG4, SPARCL1, SPINT1, SPNS2, SPOCD1, SPRN, SPRY1, SPRY2, STAMBPL1, STIP1, STMN1, SUGCT, TBC1D8, TCAF2, TCP10L, TENM3, TEX14, TFCP2L1, TFPI2, TGFB2, THBS1, THRSP, TIPARP, TMCC1, TMEM164, TMEM37, TMPRSS2, TMSB4X, TMTC1, TNFRSF21, TNS3, TP53INP1, TP63, TPRG1, TPRG1-AS1, TRAM2, TRIM31, TRIM55, TSKU, TSPAN5, TTPA, TYRO3, UBALD2, UCHL1, UGT1A7, USP12, VENTX, VLDLR, VWCE, YPEL2, ZBTB16, ZFAND2A, ZFP42, ZMYND8, ZNF292, ZNF485, ZNF608, ZNF703, ZNF793, ZNF837, and ZXDB.

Papaverine was observed to significantly (p-value≤0.05) modulate expression of A1BG-AS1, ABALON, ABCA10, ABCB11, ABLIM3, ACAA2, ACACB, ACAT2, ACTA1, ADCY7, ADGRG6, ADM, ADRB2, AHSG, AKAP12, AKR1B15, AKR1D1, ALDH8A1, ALPK2, AMACR, ANGPTL8, ANKRD35, ANO1, ANXA1, ANXA13, APOA1-AS, AQP4, AQP7, ARHGEF26-AS1, ARHGEF3, ARL14, ARL4C, ARL5B, ATP5E, ATP6V1C2, BAG3, BMF, BSN, C11orf96, C18orf32, C1orf116, C5orf45, C5orf49, C6orf201, C8orf4, CA12, CACTIN-AS1, CADPS2, CAMK2G, CASKIN1, CAT, CBLB, CBR3, CCDC152, CCDC85B, CCL20, CCL5, CD274, CDKN1C, CEBPB-AS1, CEBPD, CFAP73, CHST9, CLDN4, CLVS1, CNTD1, COL1A1, CPS1, CREB3L3, CRY2, CRYM, CSPG4, CTGF, CX3CL1, CXCL11, CXCL2, CXCL3, CYP1A1, CYP1A2, CYP26A1, CYP2C9, CYP3A4, CYP3A5, CYP7A1, CYTH1, DDIT4L, DEDD2, DERL3, DGKH, DNAH1, DNAJA4, DNAJB1, DNAJC12, EDARADD, EFNA1, EFNA3, ELOVL2, ENO3, ENTPD1, ENTPD7, EPAS1, EPHA4, EPO, ERN1, EXTL3-AS1, F13B, FAM124A, FAM219A, FAM222A, FAM49A, FASN, FAT1, FCAMR, FGF2, FKBP5, FLNA, FLNC, FLVCR2, FOS, FOSB, FOSL1, FSTL3, G6PC, GADD45B, GALNT12, GDF15, GDF9, GHR, GNAO1, GREM1, GREM2, GSG1, HAO2, HAVCR1, HES4, HHAT, HIVEP2, HSDL2, HSPA1A, HSPA1B, HSPA5, HSPA6, HSPB8, HSPH1, ICOSLG, ID2, IFT80, IGF1, IGFALS, IGFBP1, IGFBP3, IL12RB1, IL18, IL22RA1, IL6R, IRS2, ITGA3, ITGA7, ITGB6, ITIH4-AS1, JCHAIN, KCNJ8, KCNK5, KCNQ1OT1, KCNV1, KCP, KDM3A, KIAA1462, KIF12, KLF2, KLF5, KLF9, KLHL31, KLK4, KRT7, LAMB1, LAMB3, LAMC2, LBH, LECT2, LGALS4, LGALS8-AS1, LINC00102, LINC00261, LINC00313, LINC00504, LINC01098, LINC01587, LMCD1, LOC100128494, LOC100129931, LOC100133286, LOC100289473, LOC100506321, LOC101928525, LOC101928595, LOC101928597, LOC101929415, LOC101929516, LOC104968399, LOC153910, LOC440300, LPCAT3, LRRIQ3, LUCAT1, LYPD1, MAFB, MAFF, MAGEA10, MAP3K8, MATN2, MCM7, MED4-AS1, MIR1282, MIR3661, MIR5010, MIR6087, MIR6723, MIR8075, MMP24, MOG, MPHOSPH6, MT1E, MT1HL1, MT1X, MTRNR2L4, MXD3, MXRA8, MYL9, MYLIP, MYRIP, NAMPT, NAV2-AS2, NBPF20, NCF2, NCKAP5, NDRG4, NEURL3, NLGN2, NPTX2, NR1D1, NRAP, NRBP2, NUCB1-AS1, NUDT18, OSGIN1, OTUD3, OXTR, PABPC3, PALMD, PCK1, PCSK9, PDK4, PER1, PEX5L, PFKFB3, PFKFB4, PID1, PIK3CD, PITRM1-AS1, PLCG1-AS1, PLIN2, PLIN4, PLK2, PLPP3, POTEE, PPFIA4, PREX1, PRR15, PRR23C, PRSS23, PSORS1C3, PTPRU, RARB, RASD1, RBP5, RCL1, RDH12, RGS2, RHOB, RHOF, RMRP, RND1, RND3, RNVU1-19, RORA, RPL13AP6, RPL17, RPPH1, RTP3, S100A6, SCARNA10, SCD5, SCGN, SCML4, SCN11A, SEC14L2, SGSM1, SLC16A7, SLC17A1, SLC18A2, SLC25A20, SLC2A2, SLC38A4, SLC4A8, SLC6A1, SLC7A5, SLCO2B1, SMAP2, SMARCA5-AS1, SMG1P3, SNAI1, SNHG9, SNX22, SNX29, SOCS2, SOX9, SPP1, SPRN, SPRY2, SPTBN4, SPX, STAMBPL1, STOM, SULT2A1, TBC1D8, TCP10L, TDO2, TGFB2, TGFBI, THBS1, THRSP, TIPARP, TMPRSS2, TMSB4X, TNC, TNFAIP2, TNFAIP3, TRAM2, TRIM31, TSLP, TTPA, TUBA4A, TUBA4B, UBALD2, UBD, UBXN7, UGDH-AS1, UGT1A4, UGT2B4, USP12, VIM, VIM-AS1, VSIG1, VWCE, YPEL2, ZACN, ZFAND2A, ZFP42, ZHX1-C8orf76, ZNF292, ZNF485, ZNF543, ZNF608, ZNF702P, ZNF703, ZNF793, and ZXDB.

Xav939 was observed to significantly (p-value≤0.05) modulate expression of ABALON, ABCB11, ABCG2, ABLIM3, ACAA2, AGRN, AHNAK2, ALAS1, AMACR, ANKRD30BL, APOA1-AS, ARL5B, ATP6V1C2, BAG3, BCAT2, BIRC3, BMF, C5orf45, CASKIN1, CBLB, CCDC152, CCL20, CLSTN1, CPS1, CTAGE8, CXCL2, CYBB, CYP1A1, CYP1A2, CYP1B1, CYP3A5, DGKH, DNAJC12, DUSP6, ECH1, ENO3, FAM25A, FBXW10, G0S2, GDF15, GDNF, HAO2, HELZ2, HMGCS1, HSD17B7, HSPA5, HSPB1, HSPB8, ID2, IFITM1, IGFALS, IGFBP1, IGLL5, IL17D, ITGB6, KCNQ1OT1, KCNV1, KCP, KDM3A, KIAA0754, KLHDC7B, KLHL13, KRT7, KRT81, KSR2, LAMB3, LAMC2, LDLRAD4, LINC00102, LINC00504, LINC00886, LINC01104, LMCD1, LOC101929415, LOC101929448, LOC104968399, LOC105369332, LOC729348, LOXL4, LPCAT3, LRP8, LUCAT1, LYPD1, MATN2, MB21D2, METRN, MIR1282, MIR6087, MIR663AHG, MIR6723, MLK7-AS1, MMP24, MSC, MSMO1, MT1HL1, MYL9, MYOF, NCR3LG1, NMB, NRAP, NUDT18, OAS1, OTUD3, OXTR, P4HA1, PALMD, PBXIP1, PDGFB, PDLIM7, PEG10, PHLDA1, PID1, PIGR, PRSS23, RBP5, REEP1, RHCG, RHOF, RLF, RMI1, RMRP, RND3, RNVU1-19, RORA, RPPH1, RRAD, S100A6, SCARNA5, SDS, SERPINE2, SFN, SFRP4, SLC16A6, SLC23A2, SLC2A2, SLC7A11, SNORD100, SNORD12B, SNORD27, SNX22, SORBS2, SYDE2, TBC1D8, TDO2, TGFB2, TGFBI, TIPARP, TLR10, TMSB4X, TNFAIP2, TSPEAR-AS1, TUBA4A, UCN, UGCG, UGDH-AS1, USP12, VIM, ZNF292, ZNF485, and ZNF608.

Valproate was observed to significantly (p-value≤0.05) modulate expression of ABALON, ACAD11, ACAT2, ACTG2, ADRA1B, AGRN, AHNAK2, ANKRD29, ANKRD36, ANXA8L1, ATAD3C, ATP5E, ATP5EP2, BAHCC1, BMS1P5, C11orf96, C19orf71, CCDC152, CDH12, CHST9, CLEC2D, CLVS1, COX16, CX3CL1, CXCL14, CXCL3, DSG1, EGOT, EIF4EBP3, FAM49A, FAXC, FGFR3, GALNT12, GDF7, HELZ2, HMCN2, HMGCS1, HSD17B7, HSPG2, IFT80, IGFALS, IGLL5, JCHAIN, KIAA0226L, KIAA1462, KRT42P, LAMB3, LAMC2, LILRA5, LINC00102, LINC01057, LOC100128494, LOC100506675, LOC101929148, LPA, LUCAT1, MLK7-AS1, MN1, MRO, MT1E, MT1HL1, MTRNR2L3, NAV2-AS2, NBPF20, NEUROD1, NUAK2, NUDT3, PAPLN, PFDN4, PIGR, PRR23C, RDH5, RNF122, RPL13AP6, RPSAP9, RRAD, SLITRK4, SNHG9, SNORA25, SNORD17, SNORD56, SORL1, SPRR1A, SPRR3, TMEM97, TMSB4X, TNS2, TXNDC5, and ZNF702P.

Prednisone was observed to significantly (p-value≤0.05) modulate expression of A1BG-AS1, AASS, ABALON, ABCA10, ABCA4, ABCA6, ABCA9, ABCB11, ABCB9, ABCC11, ABCG1, ABCG2, ABHD2, ABLIM3, ACAA2, ACACB, ACADVL, ACAT2, ACKR2, ACMSD, ACSL1, ACSM2A, ACSM2B, ACTA1, ACTA2, ACTBL2, ADAM19, ADAMTSL2, ADGRF4, ADGRG2, ADGRG6, ADGRV1, ADM, ADRA1A, ADRB2, AFAP1L1, AGRN, AHNAK2, AHSG, AKAP12, AKR1B1, AKR1B15, AKR1D1, ALAS1, ALDH1L1, ALDH1L1-AS2, ALDH8A1, ALPK2, AMACR, ANGPTL2, ANGPTL4, ANGPTL8, ANKRD33B, ANKRD37, ANO1, ANO3, ANXA1, AOC4P, APLN, APOA1-AS, APOA4, APOBEC3B, AQP7, ARG1, ARHGEF26, ARHGEF26-AS1, ARID3C, ARL14, ARL5B, ARMC12, ARRDC3, ARSI, ARTS, ASB9, ATAD3C, ATOH8, ATP5E, ATP6V1C2, AZGP1P1, B3GNT5, BAIAP2L2, BATF, BCAS1, BCL2, BCL2A1, BDKRB2, BIRC3, BMP2, BMP8B, BNIP3, BSN, BSPRY, C10orf128, C10orf35, C10orf54, C11orf86, C11orf96, C18orf32, C1orf106, C1orf226, C2CD4A, C2orf82, C4orf47, C5AR1, C5AR2, C5orf45, CA12, CA2, CA9, CACHD1, CACTIN-AS1, CADPS2, CAMK1G, CAMK2B, CAPN8, CASKIN1, CASP16P, CBLB, CBR3, CCBL1, CCDC152, CCDC183, CCDC73, CCDC85B, CCL2, CCL20, CCL5, CCND2, CCNE2, CCR1, CD109, CDH12, CDKL1, CDKL5, CEACAM6, CEBPD, CELSR3, CEP57, CES4A, CES5A, CHST13, CHST3, CIDEC, CIT, CITED2, CLDN2, CLDN4, CLRN3, CLSTN1, CLSTN3, CLVS1, CLVS2, CNN1, CNTLN, COL10A1, COL12A1, COL15A1, COL16A1, COL1A1, COL7A1, CPA4, CPB2-AS1, CPS1, CPT1A, CPT2, CRACR2B, CREB3L3, CREB5, CRIP3, CRY2, CRYAB, CRYM, CSPG4, CTAGE8, CTGF, CUX2, CWH43, CX3CL1, CXCL2, CXCL3, CXCL8, CYB561A3, CYP21A1P, CYP21A2, CYP26A1, CYP2C19, CYP2C9, CYP3A4, CYP3A43, CYP3A5, CYP3A7, CYP3A7-CYP3A51P, CYP7A1, CYP8B1, DCLK1, DDIT4L, DEFB132, DERL3, DGKH, DHRS2, DIO3, DIRAS2, DNAH11, DNAH5, DNAI1, DNAJB4, DOCK10, DOK7, DPPA4, DSG1, DUOX2, DUOXA2, DUSP13, DYNLL1, DZIP1L, EAF2, ECH1, ECM2, EDARADD, EDN1, EFCAB1, EFNA1, EFNA3, EFR3B, EGOT, ELOVL2, EML5, EMP2, ENG, ENO2, ENO3, EPAS1, EPB41L1, EPHB2, EPO, EPPK1, EPSTI1, ERRFI1, ESR1, ESRG, ETNK2, ETNPPL, EVA1A, EXTL3-AS1, F13B, F2RL2, FADS1, FAM110A, FAM129A, FAM134B, FAM13A, FAM150B, FAM151A, FAM162A, FAM219A, FAM222A, FAM25A, FAM47E, FAM69C, FAM83D, FASN, FAT1, FAT3, FBLN5, FBXO41, FCAMR, FCRLA, FDPS, FER1L4, FGF14, FGF2, FIBIN, FKBP5, FLNA, FLNC, FLRT3, FLVCR2, FOSL1, FOXC1, FSTL1, FSTL3, FYB, G0S2, G6PC, GADD45B, GALNT15, GAREM1, GBP4, GBPS, GCNT2, GDF15, GDNF, GEM, GHR, GHRHR, GLIS3, GMNC, GOLGA6L3, GOLGA7B, GP1BA, GP6, GPER1, GPR153, GPR183, GPR88, GREM1, GREM2, GRIA3, GRIN3A, GSTP1, GUCY1B3, GYS2, HAL, HAO2, HAS3, HAVCR1, HBEGF, HELZ2, HGD, HHAT, HIPK2, HK2, HKDC1, HMCN1, HMCN2, HMGB2, HMGCS1, HMOX1, HNF4A-AS1, HOGA1, HOMER1, HPGD, HRCT1, HS1BP3-IT1, HSD3B2, HSDL2, HSP90AA1, HSPA12A, HSPA1A, HSPA1B, HSPA5, HSPB1, HSPB8, HSPH1, ICOSLG, ID1, ID2, IDI1, IER3, IFI35, IFI44L, IFIT1, IFIT2, IFITM1, IFT80, IGF1, IGFALS, IGFBP1, IGFBP3, IGSF23, IHH, IL17RB, IL18, IL22RA1, IL36B, IL6R, IL7R, ILDR2, INHBA, INHBB, INSIG1, IP6K3, IRF4, IRF5, IRF7, IRS2, ISG15, ISM1, ITGA3, ITGA5, ITGA7, ITGAM, ITGB6, ITIH4-AS1, IVL, JDP2, KANK1, KCNE1, KCNJ11, KCNJ3, KCNJ8, KCNK5, KCNQ1OT1, KDELR3, KDM3A, KIAA0895L, KIAA1462, KLF15, KLF2, KLF5, KLF9, KLHDC7B, KLHL13, KLHL31, KLK4, KRT42P, KRT7, KRT80, KRT81, KSR2, LAMA3, LAMA5, LAMB1, LAMB3, LAMB4, LAMC2, LBH, LCN2, LDB3, LDHA, LDLRAD4-AS1, LECT2, LGALS1, LGALS4, LHFPL5, LIF, LINC00102, LINC00152, LINC00261, LINC00504, LINC00595, LINC00661, LINC00862, LINC00880, LINC00881, LINC00886, LINC00987, LINC01012, LINC01016, LINC01018, LINC01126, LINC01151, LINC01314, LINC01537, LINC01554, LINC01559, LINC01587, LMCD1, LOC100128494, LOC100129931, LOC100130111, LOC100133286, LOC100506321, LOC100507283, LOC100507389, LOC101927686, LOC101927843, LOC101927973, LOC101928118, LOC101928858, LOC101929415, LOC101929427, LOC101929516, LOC101929524, LOC101929567, LOC101929574, LOC102724050, LOC102724153, LOC102724539, LOC102724652, LOC104968399, LOC105369486, LOC145694, LOC153910, LOC154761, LOC283731, LOC283887, LOC284454, LOC440300, LOC728752, LOC729348, LOX, LOXL4, LPAL2, LPIN2, LRAT, LRP2, LRP4, LRRC25, LRRC37A3, LRRC8C, LRRN2, LTBP1, LTBP3, LUCAT1, LYPD1, MAFB, MAFF, MAMDC4, MAMLD1, MANEA-AS1, MAOA, MAP1LC3B2, MAPK12, MATN2, MCAM, MCM7, MEGF6, METRN, MFNG, MGC12916, MGC32805, MILR1, MIR1282, MIR210HG, MIR3646, MIR3661, MIR4435-2HG, MIR6087, MIR663A, MIR663AHG, MIR6723, MIR6843, MIR6883, MLK7-AS1, MLLT11, MMP12, MMP24, MMP3, MMP9, MN1, MOGAT1, MOGAT3, MPEG1, MRC1, MSC, MST1R, MT1E, MT1HL1, MT1X, MTRNR2L4, MVD, MXD3, MXRA8, MYEOV, MYL9, MYLIP, MYOF, MYOM1, MYPN, MYRIP, NAGS, NAMPT, NAV3, NBPF10, NBPF20, NBPF25P, NCALD, NCF2, NCKAP1L, NCR3LG1, NDRG1, NDRG4, NDUFA4L2, NEB, NES, NETO2, NEURL3, NFASC, NLRP6, NMB, NOVA1, NPAS2, NPPB, NPR1, NPR3, NR6A1, NRAP, NRBP2, NRG4, NTSM, NTF3, NTN1, NUAK2, NUCB1-AS1, NUCB2, NXPH4, NYAP1, OAS1, OAS2, OAS3, OASL, OR4F13P, OSGIN1, OSR1, OXTR, P4HA1, PABPC3, PADI1, PALMD, PAM, PAPLN, PAQR7, PBX1, PBXIP1, PCA3, PCDH20, PCED1B, PCK1, PDE4D, PDGFB, PDGFRB, PDIA4, PDK1, PDK4, PDLIM7, PEG10, PER1, PFKFB3, PFKFB4, PGD, PGM2, PID1, PIGR, PIK3C2G, PIK3CD, PITRM1-AS1, PKHD1, PLA2G6, PLCE1, PLIN2, PLIN4, PLK2, PLPP3, PLXNA4, PLXND1, PMAIP1, PNLIPRP3, PNPLA5, POR, POTEM, POU5F1, PPARGC1A, PPFIA4, PPL, PPM1L, PPP1R14C, PPP1R3C, PPP1R3G, PRG4, PRODH, PROKR1, PROX1, PRR15, PRR23C, PRSS23, PSD3, PSORS1C3, PTHLH, PTK2B, PTPRC, PTPRU, PWAR5, QPCT, RAB17, RAB27B, RAB3B, RAB3IL1, RASD1, RASL10B, RASSF2, RBP5, RCL1, RCN3, RDH5, REEP1, RGS20, RGS4, RHCG, RHOB, RHOF, RIMKLA, RLF, RMI1, RMRP, RND1, RND3, RNVU1-19, ROBO4, ROR1-AS1, RORA, ROS1, RPGR, RPL13AP6, RPL17, RPPH1, RRAD, RSAD2, RTP3, S100A2, S100A6, SALL1, SCARA3, SCARNA17, SCD5, SCGN, SCN1B, SCN8A, SCPEP1, SDS, SEC14L2, SEC14L4, SELM, SEMA7A, SEPT4-AS1, SERHL2, SERPINA2, SERPINB7, SERPINE2, SESN3, SFN, SFRP4, SH3GLB2, SIPA1L2, SLAMF7, SLC16A12, SLC16A13, SLC16A3, SLC18A2, SLC1A2, SLC22A10, SLC22A25, SLC23A2, SLC25A18, SLC25A30, SLC2A2, SLC30A2, SLC37A4, SLC38A2, SLC38A4, SLC45A2, SLC4A5, SLC4A8, SLC5A11, SLC6A1, SLC6A6, SLC7A11, SLC7A2, SLC7A5, SLED1, SLITRK3, SMAD6, SMAP2, SMCO3, SMG1P1, SMG1P3, SMOC1, SNAI2, SNCG, SNHG9, SNORA71C, SNORD12B, SNORD17, SNORD27, SNORD49A, SOAT2, SOCS1, SOCS2, SOCS2-AS1, SORBS1, SORBS2, SORL1, SOX9, SP5, SPAG4, SPARCL1, SPINT1, SPNS2, SPOCD1, SPP1, SPRR1A, SPRR3, SPRY1, SPRY2, SPTBN4, SPTBN5, SPX, SQLE, SRPX, SSUH2, STC2, STIP1, STMN1, STOM, SUGCT, SULT1B1, SULT1E1, SULT2A1, SUSD3, SYDE2, SYPL2, SYTL2, SYTL4, TAPT1-AS1, TAT, TBC1D8, TBX15, TCAF2, TDO2, TEAD1, TEAD4, TFCP2L1, TFRC, TGFB2, TGFBI, TGFBR3, TH, THBS1, THRSP, TIPARP, TMC7, TMCC1, TMEM100, TMEM138, TMEM164, TMEM217, TMEM229B, TMEM37, TMEM47, TMEM51, TMPRSS2, TMSB4X, TMTC1, TNC, TNFAIP2, TNFAIP3, TNFRSF19, TNFRSF21, TNFSF10, TNIK, TNS2, TNS3, TOX2, TP53INP1, TP63, TPRG1, TPRG1-AS1, TRABD2B, TRAF5, TRAM2, TRIM10, TRIM55, TRIM63, TRPM8, TRPV3, TSC22D3, TSKU, TSLP, TSPAN15, TSPAN2, TSPAN5, TSPAN7, TSPEAR-AS1, TTBK1, TTYH3, TUBA1A, TUBA4A, TUBA4B, TXNIP, UBALD2, UBASH3B, UBD, UBR5-AS1, UBXN7, UGDH-AS1, UGT1A4, UGT2B4, ULBP2, UNC5CL, UPP2, VIM, VIM-AS1, VLDLR, VWCE, WSCD1, XAF1, YPEL1, YPEL2, ZACN, ZBTB16, ZC3HAV1L, ZFP42, ZMIZ1-AS1, ZMYND8, ZNF292, ZNF485, ZNF608, ZNF624, ZNF648, ZNF702P, ZNF793, and ZXDB.

Penicillamine(D−) was observed to significantly (p-value≤0.05) modulate expression of A1BG-AS1, ABALON, ABCA10, ABCC11, ABLIM3, ACAA2, AKAP12, AKR1B15, AKR1D1, AMACR, ANGPTL2, ANKRD37, ANXA1, APOA1-AS, ARHGEF26-AS1, ARL5B, ATP6V1C2, C5orf45, CACTIN-AS1, CASKIN1, CBR3, CCDC152, CCDC73, CLSTN3, CLVS1, CPS1, CPT2, CREB3L3, CX3CL1, CYB561A3, CYP26A1, CYP2C9, DNAH11, EDARADD, EFNA1, EFNA3, EGOT, ENO2, EPAS1, EPO, ETNPPL, EXTL3-AS1, F13B, FADS1, FAM110A, FAM13A, FAM222A, FAM69C, FASN, FBXO41, FER1L4, FGF2, FLNC, G0S2, G6PC, GDF15, GHR, GREM2, HAO2, HELZ2, HGD, HMGCS1, HSP90AA1, HSPB1, HSPB8, HSPH1, ID2, IGFBP1, IGFBP3, IL18, IRF7, ITGA5, ITGA7, ITIH4-AS1, KANK1, KCNJ8, KCNQ1OT1, LAMB4, LINC00504, LOC100128494, LOC100129931, LOC100133286, LOC100506321, LOC101929516, LOC104968399, LOC284454, LOX, LPIN2, LUCAT1, LYPD1, MAMDC4, MIR1282, MIR3661, MIR6087, MIR6723, MLK7-AS1, MMP24, MT1E, MT1HL1, MT1X, MTRNR2L4, MXD3, NAMPT, NBPF20, NCR3LG1, NDRG1, NDUFA4L2, NRAP, NUCB1-AS1, NXPH4, OAS3, OASL, PCK1, PER1, PFKFB3, PFKFB4, PGD, PID1, PIK3CD, PITRM1-AS1, PPFIA4, PROX1, PRSS23, PWAR5, RASD1, RMRP, RNVU1-19, RPL13AP6, RPL17, RPPH1, RTP3, S100A6, SCD5, SCGN, SCPEP1, SFN, SLC1A2, SLC22A25, SLC2A2, SLC37A4, SLC38A2, SLC38A4, SLC6A1, SLC7A2, SNAI2, SNORD27, SNORD49A, SORBS1, SPAG4, SPINT1, SPP1, STOM, SULT2A1, SYDE2, TBC1D8, TDO2, THBS1, TIPARP, TMCC1, TMEM138, TMSB4X, TNFAIP3, TSLP, TSPEAR-AS1, TUBA4A, UBALD2, UBXN7, UGT2B4, VWCE, XAF1, ZNF292, ZNF485, ZNF702P, ADCY5, BZRAP1-AS1, CBLN3, CEBPB-AS1, CRTAM, CYBB, DNAH1, ERN1, FAXC, HES4, HLA-G, IDI2-AS1, IGLL5, IL12RB1, KCP, KIAA0754, LINC00536, LINC00598, LINC01057, LINC01347, LOC100289473, LOC101928525, MAP2K6, MED4-AS1, MTMR11, NLGN2, NR1D1, PCSK9, PFN1P2, RAET1E, RGS9, RNF122, RNF186, SEC14L3, SLC25A20, SMARCA5-AS1, SNX22, STAMBPL1, USP12, and ZBTB37.

Disopyramide was observed to significantly (p-value≤0.05) modulate expression of A1BG-AS1, ABALON, ABCC11, ABLIM3, ACAA2, AKAP12, AKR1B15, AKR1D1, AMACR, ANGPTL2, ANKRD37, ANXA1, APOA1-AS, ARHGEF26-AS1, ARL5B, ATP6V1C2, C5orf45, CACTIN-AS1, CASKIN1, CBR3, CCDC152, CLSTN3, CLVS1, CPS1, CYP26A1, CYP2C9, EFNA3, ENO2, EPAS1, ETNPPL, F13B, FAM110A, FAM13A, FAM69C, FBXO41, FER1L4, FGF2, G6PC, GDF15, GHR, GREM2, HAO2, HMGCS1, HSP90AA1, HSPB1, HSPB8, HSPH1, ID2, IGFBP1, IL18, ITGA5, ITGA7, ITIH4-AS1, KANK1, KCNJ8, KCNQ1OT1, LINC00504, LOC100128494, LOC100129931, LOC100133286, LOC101929516, LOC104968399, LOC284454, LPIN2, LUCAT1, LYPD1, MAMDC4, MIR1282, MIR6723, MLK7-AS1, MMP24, MT1E, MT1HL1, MT1X, MTRNR2L4, MXD3, NAMPT, NBPF20, NCR3LG1, NDRG1, NDUFA4L2, NRAP, NUCB1-AS1, PCK1, PER1, PFKFB3, PFKFB4, PID1, PIK3CD, PITRM1-AS1, PPFIA4, PROX1, PRSS23, PWAR5, RASD1, RPL13AP6, RPL17, RTP3, S100A6, SCD5, SCGN, SLC1A2, SLC22A25, SLC2A2, SLC37A4, SLC38A2, SLC6A1, SLC7A2, SNORD27, SORBS1, SPAG4, SPP1, STOM, SULT2A1, SYDE2, TBC1D8, TDO2, THBS1, TIPARP, TMCC1, TMEM138, TMSB4X, TNFAIP3, TSPEAR-AS1, TUBA4A, UBALD2, UBXN7, UGT2B4, VWCE, XAF1, ZNF292, ZNF702P, ADCY5, CEBPB-AS1, DNAH1, ERN1, HES4, IGLL5, KCP, KIAA0754, LINC01347, LOC100289473, LOC101928525, MAP2K6, MED4-AS1, MTMR11, NLGN2, NR1D1, PCSK9, RGS9, RNF122, RNF186, SLC25A20, SNX22, USP12, ZBTB37, ABCB11, ABHD2, ACACB, ACTA2, ADGRV1, ALAS1, ALDH8A1, ALPK2, ANGPTL4, ANO1, APOA4, AQP7, ARG1, ARL14, ATAD3C, BCAS1, BNIP3, BSN, CA9, CADPS2, CBLB, CEBPD, COL1A1, CRYM, CSPG4, CXCL3, CYP2C19, CYP3A4, CYP3A5, CYP8B1, DGKH, ECH1, ELOVL2, ENO3, FAM134B, FAM162A, FAM219A, FAM47E, FKBP5, FOSL1, GADD45B, GALNT15, GPER1, HAVCR1, HK2, HMOX1, HNF4A-AS1, HSPA5, ID1, IFT80, IGFALS, IGSF23, IL22RA1, IL6R, ILDR2, IRS2, ISM1, ITGB6, KCNK5, KDM3A, KIAA0895L, KLF2, KLF5, KLF9, KLHL31, KRT7, KRT80, LAMC2, LDHA, LINC00261, LINC01018, LINC01314, LINC01587, LOC100507389, LOC101929415, LOC154761, LOC440300, LOC729348, LOXL4, LRRC37A3, MAFB, MAFF, MATN2, MCM7, METRN, MIR663A, MOGAT3, MXRA8, MYL9, MYOM1, NEB, NRBP2, NUCB2, OAS1, OXTR, P4HA1, PALMD, PAM, PDGFB, PDIA4, PDK1, PEG10, PLIN4, PLK2, PMAIP1, PPP1R3G, PRR23C, PSD3, PSORS1C3, RAB17, RAB3B, RIMKLA, RLF, RND1, RORA, RRAD, SCARNA17, SDS, SERPINE2, SLC16A3, SLC18A2, SLC23A2, SLC4A8, SLC7A11, SLC7A5, SNHG9, SNORA71C, SOCS2, SPRY2, SPTBN4, TBX15, TCAF2, TEAD1, TFRC, TGFB2, THRSP, TNS3, TRAM2, TUBA4B, UBD, UGDH-AS1, UGT1A4, VIM-AS1, VLDLR, YPEL1, YPEL2, ZC3HAV1L, ZFP42, ZNF608, ZNF793, ANKRD29, ANKRD30BL, APBB3, AQP4, ARL4C, C10orf11, CACYBP, CAMKMT, CCDC189, CDCA2, CHST9, COLCA2, CTDSP1, CXCL11, CYP1A1, CYTH1, DUSP6, EML6, FAM49A, FAM65B, FOS, FOSB, GALNT12, GIPR, HIVEP2, HMGCR, KIF12, LINC00313, LOC100507195, LOC105369332, LOC401554, LPCAT3, LRP1, MAGEA10, MANF, MAP3K8, MEI4, MIR5010, MOG, MPHOSPH6, MSMO1, NIPAL4, OTUD3, PELI2, PHLDA1, PLCG1-AS1, PROCR, RARB, RDH12, S100A9, SLC16A6, SLC16A7, SLCO2B1, SLCO4C1, SOGA3, SPRN, TENM1, TMEM45A, TTPA, UNC93A, ZNF221, ZNF80, and ZNF837.

Rapamycin was observed to significantly (p-value≤0.05) modulate expression of A1BG-AS1, ABALON, ABCC11, ABLIM3, ACAA2, AKAP12, AKR1D1, AMACR, ANKRD37, ANXA1, APOA1-AS, ARHGEF26-AS1, ARL5B, ATP6V1C2, C5orf45, CASKIN1, CBR3, CCDC152, CLSTN3, CLVS1, CPS1, CYP26A1, CYP2C9, EFNA3, ENO2, EPAS1, ETNPPL, F13B, FAM13A, FAM69C, FGF2, G6PC, GDF15, GHR, GREM2, HAO2, HMGCS1, HSPB1, HSPB8, HSPH1, ID2, IL18, ITGA7, ITIH4-AS1, KANK1, KCNJ8, KCNQ1OT1, LINC00504, LOC100128494, LOC100129931, LOC101929516, LOC104968399, LOC284454, LPIN2, LUCAT1, LYPD1, MAMDC4, MIR1282, MIR6723, MLK7-AS1, MMP24, MT1E, MT1HL1, MT1X, MTRNR2L4, MXD3, NAMPT, NBPF20, NCR3LG1, NDRG1, NRAP, PCK1, PER1, PFKFB3, PFKFB4, PID1, PIK3CD, PITRM1-AS1, PPFIA4, PROX1, PRSS23, RPL13AP6, RPL17, RTP3, S100A6, SCD5, SLC22A25, SLC2A2, SLC6A1, SNORD27, SORBS1, SPAG4, SPP1, STOM, SULT2A1, TBC1D8, TDO2, THBS1, TIPARP, TMCC1, TMSB4X, TNFAIP3, TUBA4A, UBALD2, UBXN7, UGT2B4, VWCE, ZNF292, HES4, KCP, KIAA0754, MAP2K6, MTMR11, NR1D1, PCSK9, RGS9, RNF186, SLC25A20, SNX22, USP12, ZBTB37, ABCB11, ABHD2, ACTA2, ADGRV1, ALAS1, ALDH8A1, ALPK2, ANGPTL4, ANO1, APOA4, ARG1, ARL14, BCAS1, BNIP3, CADPS2, COL1A1, CSPG4, CYP2C19, CYP3A4, CYP3A5, CYP8B1, ELOVL2, ENO3, FAM134B, FAM162A, FAM47E, FOSL1, GADD45B, HK2, HMOX1, IFT80, IGFALS, IGSF23, IL22RA1, IL6R, ILDR2, ITGB6, KCNK5, KDM3A, KLF2, KLF5, KLF9, KLHL31, KRT7, KRT80, LAMC2, LDHA, LINC00261, LINC01018, LINC01314, LOC100507389, LOC101929415, LOC440300, LOC729348, LOXL4, LRRC37A3, MAFF, MATN2, METRN, MIR663A, MOGAT3, MXRA8, MYL9, MYOM1, NUCB2, OAS1, OXTR, P4HA1, PALMD, PAM, PDGFB, PDIA4, PDK1, PEG10, PLIN4, PLK2, PPP1R3G, PSD3, PSORS1C3, RAB17, RAB3B, RIMKLA, RLF, RND1, RORA, RRAD, SDS, SERPINE2, SLC23A2, SLC4A8, SLC7A11, SOCS2, SPTBN4, TCAF2, TFRC, TGFB2, THRSP, TNS3, TRAM2, TUBA4B, UGDH-AS1, UGT1A4, VIM-AS1, VLDLR, YPEL2, ZC3HAV1L, ZFP42, AQP4, ARL4C, CHST9, CXCL11, CYP1A1, CYTH1, DUSP6, EML6, FOSB, HIVEP2, HMGCR, KIF12, LOC100507195, LRP1, MAP3K8, MIR5010, MSMO1, NIPAL4, PELI2, PHLDA1, RARB, SLC16A6, SLCO2B1, SLCO4C1, SPRN, TENM1, TTPA, UNC93A, ZNF80, CCDC73, CPT2, CX3CL1, EFNA1, EPO, EXTL3-AS1, FADS1, FAM222A, FASN, FLNC, HGD, IGFBP3, MIR3661, MIR6087, OASL, PGD, RMRP, RNVU1-19, RPPH1, SFN, SLC38A4, SNAI2, TSLP, ZNF485, BZRAP1-AS1, CYBB, IL12RB1, LINC01057, PFN1P2, STAMBPL1, ABCA4, ABCA9, ABCG2, ACKR2, ACSL1, ACTA1, ACTBL2, ADAM19, ADGRF4, ADGRG6, ADM, ADRB2, AFAP1L1, AGRN, AHNAK2, AHSG, AKR1B1, ALDH1L1, ANGPTL8, ARID3C, ATP5E, AZGP1P1, BAIAP2L2, BIRC3, BMP2, BMP8B, C10orf128, C11orf96, C1orf106, C2orf82, CA12, CA2, CACHD1, CAPN8, CCL2, CCL20, CD109, CES4A, CES5A, CHST13, CHST3, CITED2, CLDN4, CLSTN1, CNN1, CNTLN, COL16A1, COL7A1, CPB2-AS1, CPT1A, CRACR2B, CREB5, CRY2, CRYAB, CTGF, CUX2, CXCL2, CYP21A2, CYP3A43, CYP3A7, CYP3A7-CYP3A51P, CYP7A1, DDIT4L, DHRS2, DNAH5, DNAJB4, DOK7, DSG1, DUSP13, DZIP1L, ECM2, EDN1, EML5, EMP2, EPHB2, EPPK1, ERRFI1, EVA1A, F2RL2, FAM151A, FAT1, FAT3, FCAMR, FDPS, FLNA, FLRT3, FLVCR2, FSTL3, GCNT2, GEM, GLIS3, GOLGA7B, GP1BA, GRIA3, GRIN3A, HAL, HAS3, HIPK2, HKDC1, HMGB2, HPGD, HRCT1, HSPA1A, ICOSLG, IDI1, IER3, IFI35, IFIT2, IGF1, IL17RB, IL7R, INHBA, INSIG1, ISG15, ITGA3, IVL, JDP2, KCNE1, KLHDC7B, KLHL13, KRT81, KSR2, LAMA5, LAMB3, LBH, LCN2, LECT2, LGALS1, LGALS4, LHFPL5, LIF, LINC00152, LINC00862, LINC00880, LINC01151, LMCD1, LOC101928118, LOC101929567, LOC283731, LPAL2, LRAT, LRRC8C, LRRN2, LTBP1, LTBP3, MAMLD1, MCAM, MGC12916, MILR1, MIR210HG, MMP12, MMP3, MN1, MOGAT1, MSC, MST1R, MVD, MYLIP, MYOF, MYRIP, NAGS, NCALD, NDRG4, NES, NETO2, NEURL3, NMB, NPAS2, NPR3, NR6A1, NUAK2, OSGIN1, PAPLN, PAQR7, PBXIP1, PCDH20, PCED1B, PDLIM7, PGM2, PIGR, PKHD1, PLPP3, PNLIPRP3, POR, PPARGC1A, PPL, PPM1L, PRR15, QPCT, RAB27B, RAB3IL1, RBP5, RCL1, RDH5, RHOF, RMI1, ROBO4, ROR1-AS1, ROS1, RSAD2, SCARA3, SCN8A, SEC14L4, SEMA7A, SESN3, SFRP4, SIPA1L2, SLC16A12, SLC22A10, SLC25A18, SLC5A11, SLC6A6, SLITRK3, SMCO3, SMG1P3, SMOC1, SORBS2, SORL1, SOX9, SPOCD1, SPX, SQLE, STC2, SUGCT, SYTL2, SYTL4, TAPT1-AS1, TAT, TFCP2L1, TGFBI, TMC7, TMEM37, TMPRSS2, TNC, TNFAIP2, TNFRSF19, TNFRSF21, TNS2, TP53INP1, TP63, TRPM8, TRPV3, TSKU, TSPAN2, TSPAN5, TSPAN7, UBASH3B, UNC5CL, VIM, ZMIZ1-AS1, ZNF648, ZXDB, AADAC, ABLIM2, ACSL5, ACSS2, ADCY7, ADRA1B, ADRB1, AGTR1, AKR1C8P, ALOX5, ANGPTL1, ANGPTL7, ANXA13, ARL4D, ATG9B, ATP6V0D2, AVPR1A, BMF, C19orf71, C1QTNF1-AS1, C2orf54, C4orf19, C4orf32, C8orf4, C9orf72, CAMK2G, CD274, CDRT1, CNTD1, COL4A2, COL4A4, CPVL, CSRNP3, CXCL12, CYP1B1, CYP24A1, CYTH4, DCX, DGKG, DNAJB1, DNAJC12, DSG2-AS1, EGLN3, ENPP2, ENTPD7, EPHA4, ERICH5, ESRRG, FABP3, FADS2, FAM124A, FAM43A, FAM46C, FBXO30, FBXW10, FBXW4P1, FDFT1, FGF13, FGF19, FGF21, FGFR3, FGR, FMO1, GAL3ST1, GBP6, GLA, GLRA2, GNAO1, GPC6, GPCPD1, HCAR2, HCAR3, HDC, HIST3H2A, HMGCS2, HSPG2, IGF2BP3, IGFBP6, IGFN1, IL12RB2, IL23A, INHBE, KIAA0226L, KIAA0319, KIF5A, KIRREL, LAG3, LDLRAD4, LEFTY1, LGALS9, LILRA5, LINC00266-3, LINC00704, LOC100505635, LOC101927630, LOC101929448, LOC101930114, LOC105371795, LOC105372833, LOC105373051, LOC254028, LOC284344, LOC344887, LPA, LRFN2, LRP8, LRRIQ3, MAATS1, MAB2IL2, MAP1B, MAP6, MCHR1, MIR4645, MMP19, MOGAT2, MPV17L, MSX1, MYH3, MYH4, NDUFA6-AS1, NECTIN4, NEFM, NEXN, NOS2, NUDT18, NUGGC, P2RY1, P4HA3, PCSK5, PFDN4, PGAM4, PKD1L2, PKD2L1, PKLR, PLAU, PLCH1, PMEPA1, PRAMEF2, PRDM7, PREX1, PROM1, RAPGEF4, RGCC, SEC14L6, SEC31B, SERPINB9, SGK2, SH3TC2, SHC4, SIX4, SLC16A14, SLC17A2, SLC29A4, SLC2A10, SLC51B, SLC6A20, SMAD5-AS1, SMIM3, SNORD33, SPOCK1, STK39, STRA6, SUSD1, TAC1, TCP10L, TFPI2, TGFB1I1, TGFB3, TLE2, TLE6, TLR10, TMEM105, TMEM156, TMEM25, TMEM97, TOX3, TPM2, TRAF3IP3, TREM1, TREML3P, TREML4, TRIM31, TUSC3, UBE2D1, UCHL1, UGCG, UGT1A3, UGT1A5, VENTX, VLDLR-AS1, VWA3B, ZFAND2A, ZNF192P1, ZNF543, and ZNF703.

Simvastatin was observed to significantly (p-value≤0.05) modulate expression of ABLIM3, AKAP12, AMACR, ARL5B, CPS1, CYP2C9, ENO2, EPAS1, F13B, GDF15, HAO2, HMGCS1, KANK1, KCNJ8, LYPD1, MMP24, MT1E, MT1HL1, MT1X, NDRG1, PFKFB4, PID1, PIK3CD, PPFIA4, PROX1, PRSS23, RTP3, S100A6, SLC2A2, SLC6A1, SPP1, STOM, SULT2A1, TDO2, THBS1, TIPARP, TMCC1, TUBA4A, UBALD2, UGT2B4, KCP, MAP2K6, MTMR11, PCSK9, RGS9, RNF186, SNX22, USP12, ABHD2, ACTA2, ALDH8A1, ALPK2, ANGPTL4, APOA4, BCAS1, COL1A1, CYP8B1, FAM134B, FAM162A, FAM47E, FOSL1, GADD45B, HMOX1, IGFALS, IGSF23, IL22RA1, KCNK5, KDM3A, KLF2, KLF5, KLF9, KLHL31, LAMC2, LINC00261, LINC01314, LOXL4, LRRC37A3, MAFF, MATN2, METRN, MOGAT3, MYL9, MYOM1, P4HA1, PALMD, PDGFB, PDIA4, PLIN4, PLK2, RAB3B, RND1, SDS, SLC7A11, SOCS2, TGFB2, THRSP, YPEL2, CHST9, CXCL11, CYTH1, DUSP6, FOSB, HMGCR, LOC100507195, MSMO1, PHLDA1, RARB, SLCO2B1, SLCO4C1, TENM1, TTPA, UNC93A, ZNF80, FADS1, FASN, HGD, IGFBP3, OASL, PGD, SFN, SLC38A4, ACSL1, AHNAK2, ALDH1L1, ARID3C, ATP5E, BMP2, BMP8B, C11orf96, C1orf106, C2orf82, CHST13, CHST3, CLDN4, CLSTN1, COL16A1, CREB5, CRYAB, CUX2, CYP7A1, DNAH5, DSG1, EDN1, EPPK1, ERRFI1, FAM151A, FDPS, FLNA, FLVCR2, GRIA3, HPGD, HRCT1, HSPA1A, IDI1, IER3, IFIT2, IGF1, IL17RB, INSIG1, IVL, KLHDC7B, KSR2, LBH, LGALS1, LGALS4, LIF, LINC01151, LMCD1, MVD, MYOF, NUAK2, PAPLN, PDLIM7, PIGR, PKHD1, RAB27B, RBP5, RHOF, SCN8A, SEMA7A, SIPA1L2, SMC03, SORBS2, SPOCD1, SQLE, STC2, TMC7, TMPRSS2, TNC, TNS2, TSKU, UBASH3B, UNC5CL, VIM, ZXDB, ACSS2, C4orf32, CYP1B1, CYP24A1, DNAJB1, FABP3, FADS2, FDFT1, FGFR3, FMO1, GLRA2, GNAO1, GPC6, HMGCS2, IL23A, LPA, LRP8, MAP1B, MMP19, MOGAT2, MPV17L, NDUFA6-AS1, NECTIN4, NUGGC, PCSK5, PKD2L1, PMEPA1, RGCC, SGK2, SLC17A2, TMEM97, TPM2, UBE2D1, UGCG, ZFAND2A, ZNF703, ANGPTL2, HSP90AA1, IGFBP1, ITGA5, NDUFA4L2, RASD1, SCGN, SLC38A2, TMEM138, ADCY5, RNF122, CBLB, CRYM, FKBP5, GPER1, MAFB, NEB, PMAIP1, SLC16A3, TBX15, ZNF608, APBB3, CAMKMT, GALNT12, LPCAT3, MEI4, SLC16A7, SOGA3, CYB561A3, EGOT, IRF7, RAET1E, ABCG1, ACAT2, ACSM2A, ACSM2B, B3GNT5, CPA4, CRIP3, DUOX2, DYNLL1, ESR1, FCRLA, HBEGF, HMCN1, HOGA1, HSPA1B, IRF4, KCNJ3, KIAA1462, KLF15, LAMB1, LINC01554, LINC01559, MEGF6, NFASC, PIK3C2G, PPP1R14C, PPP1R3C, RHCG, RHOB, RND3, S100A2, SEC14L2, SLC16A13, SPRR1A, SPRR3, SYPL2, TMEM217, TOX2, TSC22D3, TTYH3, TUBA1A, TXNIP, UPP2, ACAD11, ACOT1, AHRR, AK7, ANXA3, ARHGDIB, ATP2B2, BAG3, BCAT2, C1orf116, CACNA1D, CAPN13, CDHR1, COL4A1, DBH-AS1, DEDD2, DPY19L2, EME2, FAM198A, GBAP1, GNAZ, HSD17B7, HSPA6, KANK4, KIF3C, LBHD1, LOC100190940, LOC100506258, LOC100507002, LOC101928865, LOC105376575, LOC728040, MED31, MGAM, MRO, NCKAP5, NPTX2, PIGZ, PLCH2, PLOD1, S1PR1, SH2D3C, SLC25A42, SMIM10L2B, SMO, SNORD97, TEAD2, TENM2, TNFSF11, USP2, and VWF.

DmPGE2 was observed to significantly (p-value≤0.05) modulate expression of A1BG-AS1, ACAD11, ADGRG6, AGRN, AHSG, AKAP12, ALPK2, ANGPTL4, ANKRD30BL, APOA1-AS, ATAD3C, ATP6V1C2, C5orf45, CASKIN1, CCDC152, CDH12, CLVS1, CYP3A5, DNAJC12, DSG1, DSG2-AS1, DUOX2, ELOVL2, ERRFI1, ETNPPL, FADS1, FAM13A, FAM69C, FAT1, FBXO41, FDFT1, FDPS, FGF2, G6PC, GADD45B, GALNT12, GDF15, GDNF, GHR, GLIS3, GREM2, HAO2, HES4, HHAT, HIPK2, HKDC1, HSD17B7, HSPA1B, ID2, IFIT2, IGFBP1, IGLL5, IL23A, ISM1, ITGB6, KCNQ10T1, KCP, KIAA0895L, KIAA1462, KLHL13, KRT81, LAMC2, LINC00102, LINC00886, LINC01012, LOC104968399, LOC284454, LRRC39, LYPD1, MAMDC4, MCM7, MIR6723, MLK7-AS1, MMP12, MT1E, MT1HL1, MT1X, MYH3, NRAP, OAS1, OAS3, PER1, PEX5L, PHLDA1, PID1, PROX1, PSD3, RASD1, RASSF2, REEP1, RHOF, RND3, RPL13AP6, SCARNA7, SLC2A2, SLC37A4, SLC38A4, SLC7A2, SNORD15B, SNORD33, SNX22, SPAG4, TBC1D8, TMPRSS2, TPM2, TUBA4A, UCN, UGT2B4, VENTX, VIM, VWCE, XAF1, and ZNF485.

ATRA was observed to significantly (p-value≤0.05) modulate expression of A1BG-AS1, AADAC, ABALON, ABCA6, ABCA9, ABCB11, ABCC11, ABCG1, ABCG2, ABLIM3, ACAA2, ACSM2A, ACTG2, ADAM19, ADGRG6, ADGRV1, ADRB2, AGRN, AHNAK2, AHSG, AKR1B15, AKR1D1, ALAS1, ALDH8A1, ALPK2, AMACR, ANGPTL8, ANKRD29, ANKRD30BL, ANO1, ANXA13, AOC4P, APOA1-AS, APOBEC3A, APOBEC3B, AQP4, ARHGEF26-AS1, ARHGEF3, ARL14, ARRDC3, ARSI, ASB14, ATAD3C, ATOH8, ATP5E, ATP5EP2, ATP6V0E2-AS1, ATP6V1C2, BCAS1, BCL2A1, BIRC3, BMF, BNIP3, BSN, C10orf54, C18orf32, C1orf226, C1QTNF1-AS1, C2orf54, C2orf91, C4orf19, C4orf32, C5orf45, C8orf4, CA12, CACHD1, CACNA1D, CACYBP, CADPS2, CAMK2G, CAMKMT, CASKIN1, CAT, CBLB, CBR3, CBWD3, CCDC152, CCDC28B, CCDC73, CCDC85B, CCL2, CCL5, CD109, CD274, CDH12, CDKL1, CDKL5, CEBPB-AS1, CECR1, CELSR3, CEP57, CFAP73, CHST13, CHST9, CIITA, CLEC2D, CLEC7A, CLSTN1, CLVS1, CNN1, COL7A1, COLCA2, CPS1, CREB3L3, CREB5, CRIP3, CRYAB, CRYM, CSNK1E, CSPG4, CTAGE8, CX3CL1, CXCL2, CYP1A1, CYP1A2, CYP21A2, CYP24A1, CYP26A1, CYP3A5, CYP7A1, CYP8B1, DAND5, DCLK1, DGKH, DHRS2, DNAH1, DNAJB1, DNAJB4, DNAJC12, DSG2-AS1, DUSP6, DYNLL1, DZIP1L, ECH1, ECM2, EDARADD, EDN1, EFNA1, EFR3B, EMP2, ENO3, ENPP2, ENTPD1, EPAS1, EPB41L1, ERI3-IT1, ERN1, ERRFI1, ESRG, ESRRG, ETNPPL, EVA1A, F13B, FADS1, FAM13A, FAM162A, FAM198A, FAM219A, FAM227B, FAM25A, FAM43A, FAM47E, FASN, FBXO41, FCAMR, FGF19, FGF2, FGFR3, FHL3, FLJ43879, FLNC, FLRT3, FLVCR2, FMO1, FOSB, FSTL3, FTX, GOS2, GALNT12, GALNT15, GBP4, GDF15, GEM, GNAZ, GOLGA6L3, GPC6, GPM6A, GPRIN3, GRIA3, GSG1, GSTP1, GYS2, HAO2, HAS3, HBEGF, HELZ2, HHAT, HIPK2, HIST3H2A, HIST3H2BB, HIVEP2, HK2, HKDC1, HLA-G, HOGA1, HPGD, HSP90AA1, HSPA1A, HSPA1B, HSPA5, HSPB1, HSPB8, HSPH1, ID2, IDI1, IER3, IFIT1, IFT80, IGF1, IGFBP3, IGFBP6, IL12RB1, IL17RB, IL18, INHBB, ISG15, ITGA3, ITGA5, ITGA7, ITGB6, ITIH4-AS1, IVL, KCNB1, KCNJ11, KCNJ8, KCNK5, KCNMB4, KCNQ1OT1, KIAA0754, KIR3DX1, KLF15, KLHL13, KRT42P, KRT81, LAMA3, LAMA4, LAMA5, LAMB4, LAMC2, LECT2, LGALS1, LGALS4, LGALS9, LHFPL5, LIF, LILRA5, LINC00102, LINC00261, LINC00504, LINC00661, LINC00862, LINC00880, LINC01012, LINC01057, LINC01314, LINC01347, LINC01587, LMCD1, LOC100128494, LOC100133286, LOC100506675, LOC100507002, LOC100507389, LOC101927809, LOC101928118, LOC101928514, LOC101929148, LOC104968399, LOC153910, LOC284454, LOC401554, LOC440311, LOC728752, LOC729348, LOXL4, LPA, LPIN2, LRAT, LRFN2, LRP1, LRP8, LRRC8C, LTBP3, LYPD1, MAGEA10, MANF, MAP1LC3C, MAP3K8, MCAM, MIR1282, MIR3661, MIR4435-2HG, MIR4523, MIR6087, MIR663A, MIR663AHG, MIR6723, MIR8075, MKNK1-AS1, MLK7-AS1, MLLT1, MLLT11, MMP19, MMP3, MOGAT1, MOGAT2, MPHOSPH6, MPP2, MPV17L, MSC, MT1E, MT1HL1, MT1X, MTMR11, MTRNR2L4, MXD3, MXRA8, MYRIP, NAMPT, NBPF10, NBPF20, NBPF25P, NCF2, NCR3LG1, NEB, NEURL3, NEXN, NFATC4, NIPAL4, NLGN2, NMB, NPAS2, NPTXR, NR6A1, NRAP, NRG4, NRP2, NUCB2, NUDT18, OAS1, OR2A7, OSGIN1, OXTR, PALMD, PCDH9, PCDHGA8, PCK1, PDIA4, PDK4, PDLIM7, PEG10, PELI2, PER1, PFDN4, PGM2, PID1, PIGR, PITRM1-AS1, PKHD1, PKLR, PLA2G6, PLCXD2, PLIN2, PLPP3, PLXND1, PPFIA4, PPL, PPM1L, PPP1R3C, PREX1, PROX1, PRR15, PRSS23, PSD3, PTPRU, PTPRVP, PWAR5, RAB27B, RAB3B, RAET1E, RARB, RBP5, RCL1, RDH12, RGS9, RMRP, RNVU1-19, RPL17, RPL22L1, RPLP0P2, RPPH1, RPSAP9, RRAD, RSAD2, RTP3, S100A6, S1PR1, SCDS, SCGN, SCPEP1, SCUBE1, SDS, SEC14L2, SEMA7A, SERPINA2, SERPINB9, SERPINE2, SFN, SGK2, SH3TC2, SLC22A10, SLC22A25, SLC23A2, SLC29A4, SLC2A2, SLC38A2, SLC44A5, SLC4A8, SLC7A11, SLC7A2, SLC7A5, SLCO2B1, SLITRK4, SMC2-AS1, SMG1P1, SMG1P3, SMPD3, SNHG9, SNORD12, SNORD12B, SNORD12C, SNORD49A, SNX22, SOAT2, SOCS2, SORBS1, SORBS2, SORL1, SOX9, SPARCL1, SPOCD1, SPP1, SQLE, STIP1, STOM, SYPL2, SYTL3, SYTL4, TBC1D8, TCAF2, TDO2, TEAD1, TEKT5, TENM1, TGFB1I1, TH, THBS1, TMEM164, TMEM217, TMEM45A, TMEM47, TMPPE, TMSB4X, TNFAIP2, TNFRSF21, TNFSF10, TNRC6C-AS1, TNS2, TNS3, TPM2, TRAM2, TRIM31, TSKU, TSLP, TSPAN5, TSPO2, TTYH3, TUBA4A, TUBA4B, TXNDC5, UBD, UBE2D1, UBXN7, UCHL1, UGT1A4, UGT2B4, ULBP2, UNC5CL, UPK3B, USP2, VENTX, VIM, VIM-AS1, ZBTB37, ZC3HAV1L, ZFP42, ZMYND8, ZNF221, ZNF292, ZNF385C, ZNF702P, ZNF793, ZNF827, and ZXDB.

Isoniazid was observed to significantly (p-value≤0.05) modulate expression of A1BG-AS1, ABALON, ABCA10, ABCA9, ABCB11, ABCC11, ABHD16B, ABHD2, ABLIM3, ACAA2, ACTA1, ACTA2, ADM, AKAP12, AKR1B15, ALAS1, ALPK2, AMACR, ANGPTL2, ANGPTL8, ANXA13, APOA1-AS, APOA4, APOBEC3B, ARG1, ARHGEF26-AS1, ARL5B, ARRDC3, ATP5E, ATP6V1C2, BCAT2, BIRC3, BNIP3, C2orf91, C5orf45, CA2, CACTIN-AS1, CASKIN1, CBLB, CBR3, CCDC152, CCDC85B, CCL5, CD274, CD4, CDKL1, CEBPB-AS1, CHST13, CHST9, CLEC7A, CLVS1, CNN1, COX16, CRYM, CSPG4, CTAGE8, CXCL8, CYB561A3, CYP2C9, CYP3A5, CYTH1, DGKH, DNAH11, DOK7, DUSP6, ECH1, EDARADD, EFNA1, ENO2, ENO3, EPO, EPPK1, ETNPPL, FAM134B, FAM13A, FAM162A, FAM69C, FCAMR, FGFR3, FLNA, GBP4, GBP5, GCNT2, GNAO1, GREM2, HAO2, HGD, HHAT, HIVEP2, HK2, HMGCR, HMGCS1, HMOX1, HSD3B2, HSPA1A, HSPA1B, HSPB1, HSPB8, HSPH1, ID2, IDI2-AS1, IFIT2, IGFALS, IGFBP1, IGFBP5, IGLL5, IL18, IL23A, IL6R, INSIG1, ISM1, ITGA3, ITGA7, ITGB6, ITIH4-AS1, KANK1, KCNJ8, KCNK5, KCNQ1OT1, KDM3A, KIAA0754, KIF12, KISS1R, KRT7, KRT80, KRT81, LAMA5, LAMC2, LDHA, LGALS1, LHFPL5, LINC00102, LINC00504, LOC100128494, LOC100129931, LOC100133286, LOC100506321, LOC101927973, LOC101928504, LOC101928525, LOC101928865, LOC101929415, LOC101929516, LOC104968399, LOC440300, LOXL4, LPCAT3, LPIN2, LRAT, LRRC24, LRRIQ3, LTBP3, LUCAT1, LYPD1, MAFB, MAP3K8, MATN2, MCAM, MED31, METRN, MIR1282, MIR3661, MIR6087, MIR6723, MLK7-AS1, MMP24, MSC, MSMO1, MT1E, MT1HL1, MT1X, MTMR11, MTRNR2L4, MXD3, MXRA8, MYL9, NAV2-AS2, NBPF20, NCF2, NCR3LG1, NDRG1, NDUFA4L2, NFASC, NLRP11, NME2, NRAP, NTN1, NUAK2, NUCB1-AS1, OSGIN1, OTUD3, P4HA1, PAM, PAPLN, PDIA4, PDK1, PFKFB3, PHLDA1, PID1, PIK3C2G, PITRM1-AS1, PLCG1-AS1, PLIN4, POR, POTEE, PPFIA4, PPL, PRKRIP1, PRSS23, PSORS1C3, PWAR5, RAB3B, RARB, RGCC, RGS9, RHCG, RIMKLA, RLF, RMI1, RMRP, RND3, RNF122, RNVU1-19, ROR1-AS1, RORA, ROS1, RPL13AP6, RPL17, RPPH1, RRAD, RTP3, S100A6, S1PR1, SDS, SERPINE2, SFN, SLC1A2, SLC25A20, SLC25A42, SLC2A2, SLC37A4, SLC7A11, SLC7A2, SLCO2B1, SLCO4C1, SMARCA5-AS1, SNHG9, SNORD27, SNX22, SOCS2, SORBS1, SPAG4, SPRN, SPRY1, SPRY2, SQLE, TBC1D8, TBX15, TCAF2, TCP10L, TFRC, THBS1, TIPARP, TMCC1, TMEM138, TMEM45A, TMEM47, TMSB4X, TNC, TNFAIP3, TNFSF10, TNFSF11, TP53INP1, TRO, TSKU, TSLP, TSPEAR-AS1, TUBA4A, TUSC3, UBXN7, UGDH-AS1, USP12, VIM, VIM-AS1, VLDLR, VWCE, XAF1, YJEFN3, YPEL2, ZACN, ZBTB37, ZFP42, ZNF192P1, ZNF292, ZNF485, ZNF608, and ZNF702P.

Acetaminophen was observed to significantly (p-value≤0.05) modulate expression of ABLIM3, ACAA2, ACAD11, AHSG, AKR1B15, ALPK2, ARL14, ATAD3C, ATP5EP2, C18orf32, CBR3, CLEC2D, CSRNP3, CTDSP1, CXCL14, CYBB, CYP1A2, CYP2C9, DNAJC12, EDARADD, ERRFI1, EXTL3-AS1, FAM218A, FAM219A, FAM69C, FASN, FBXO41, FGF2, FSTL3, GADD45B, GDF15, GPR85, GREM2, HES4, HSD17B7, HSP90AA1, HSPA1A, HSPA1B, HSPA5, HSPA6, IFT80, IL7R, KCP, KIAA0895L, KIAA1462, KRT42P, LAMC2, LOC100506675, LOC728752, MIR6087, MIR663AHG, MT1E, MT1HL1, MT1X, MTRNR2L4, NEUROD1, NEUROD2, NLGN2, NLRP11, NR1D1, OSGIN1, PDLIM7, PEG10, PFDN4, PID1, PIGR, PLOD1, POU2AF1, RASD1, RHOB, RNVU1-19, RPL17, RPL22L1, RPLP0P2, RPSAP9, S100A6, S1PR1, SLC37A4, SLC4A8, SLITRK4, SNHG9, SNORA50C, SNORD56, TBC1D8, TMEM238, TMEM97, TMSB4X, TNFAIP2, TREH, TRIM69, TSLP, TTPA, TXNDC5, UBALD2, UBE2D1, UGT2B4, USP2, VENTX, and ZNF702P.

Ritonavir was observed to significantly (p-value≤0.05) modulate expression of ABALON, ABCA4, ABCA6, ABCA7, ABCA9, ABCB11, ABCG1, ABLIM2, ABLIM3, ACAA2, ACAD11, ACADVL, ACAT2, ACOT1, ACSL1, ACSL5, ACSM2A, ACSS2, ACTA1, ACTA2, ACTG2, ADCY10P1, ADCY7, ADGRG2, ADGRG6, ADGRV1, ADM, ADRB2, AFAP1L1, AGRN, AGTR1, AHNAK2, AHRR, AHSG, AKAP12, AKR1B1, AKR1B15, AKR1C8P, AKR1D1, ALAS1, ALDH1L1, ALDH8A1, ALOX5, ALPK2, AMACR, ANGPTL1, ANGPTL2, ANGPTL4, ANGPTL8, ANKRD23, ANO1, ANXA13, APOA1-AS, APOA4, APOBEC3A, AQP4, ARC, ARHGAP28, ARHGEF26, ARHGEF26-AS1, ARL14, ARL4C, ARL4D, ARL5B, ARRDC3, ATAD3C, ATG9B, ATOH8, ATP5E, ATP5EP2, ATP6V0D2, ATP6V1C2, AVPR1A, AZGP1P1, AZU1, B3GNT5, B4GALNT1, BAG3, BAHCC1, BCL2, BEX4, BIK, BIRC3, BMP2, BMP3, BMP6, BMP8B, BRF2, BSPRY, C10orf11, C10orf128, C10orf35, C10orf54, C11orf96, C19orf71, C1orf105, C1orf116, C1orf226, C1orf53, C2orf54, C2orf82, C2orf91, C4orf32, C5orf45, C8orf4, C9orf152, C9orf173-AS1, C9orf24, C9orf72, CA12, CA2, CACHD1, CACNA1D, CACYBP, CADPS2, CAMK2G, CAPN8, CASKIN1, CASP16P, CAT, CBLB, CBLN3, CBR3, CCBL1, CCDC102B, CCDC114, CCDC152, CCDC189, CCDC28B, CCDC73, CCDC85B, CCL2, CCL26, CCL5, CCNE2, CCR1, CD274, CD300LB, CDC6, CDKN1C, CEBPD, CELSR2, CELSR3, CFAP47, CHI3L2, CHST13, CHST9, CIITA, CLDN16, CLDN2, CLDN4, CLEC2D, CLRN3, CLSTN3, CLVS1, CNN1, CNTLN, CNTN2, COL12A1, COL4A1, COL4A4, COL7A1, CPB2-AS1, CPEB1, CPS1, CPT1A, CRACR2B, CREB3L3, CREB5, CRY2, CRYAB, CRYM, CSNK1E, CSPG4, CSRNP3, CTAGE8, CTDSP1, CUX2, CX3CL1, CXCL1, CXCL11, CXCL14, CXCL2, CXCL3, CXCL8, CYP1A1, CYP1A2, CYP21A2, CYP24A1, CYP26A1, CYP2C19, CYP2C9, CYP3A4, CYP3A43, CYP3A5, CYP3A7, CYP3A7-CYP3A51P, CYP7A1, CYP8B1, CYTH1, DAND5, DBH-AS1, DCX, DDIT4L, DEDD2, DEFB132, DGKG, DNAH12, DNAH5, DNAH7, DNAI1, DNAJA4, DNAJB1, DNAJB4, DNAJB5-AS1, DNAJC12, DNM1, DNMT3L, DSC1, DSG1, DSG2-AS1, DUOX2, DUOXA2, DUSP13, DUSP6, ECH1, ECM2, EDARADD, EDN1, EFCAB5, EFNA1, EFNA3, EGOT, EIF4EBP3, ELMOD1, ELOVL2, EML6, EMP2, ENO3, ENTPD7, EPAS1, EPB41L1, EPHA4, EPHB2, EPPK1, EPSTI1, ERICH5, ERN1, ETNK2, ETNPPL, EVA1A, EVPLL, F2RL2, FADS1, FADS2, FAM105A, FAM124A, FAM129A, FAM13A, FAM151A, FAM162A, FAM167A, FAM171B, FAM198A, FAM219A, FAM222A, FAM227B, FAM25A, FAM26F, FAM47E, FAM49A, FAM65B, FAM83D, FASN, FAT1, FBXO30, FBXO41, FBXW4P1, FDFT1, FDPS, FER1L4, FEZ1, FGF19, FGF2, FGFR3, FGR, FKBP5, FLNA, FLNC, FLRT3, FLVCR2, FMO1, FOS, FOSB, FOSL1, FSTL3, FYB, G0S2, G6PC, GADD45B, GAL3ST1, GALE, GALNT12, GALNT15, GAREM1, GBAP1, GBGT1, GBP6, GCNT2, GDF15, GHR, GIPR, GLA, GNAZ, GOLGA7B, GPM6A, GPRASP1, GRIA3, GSDMC, GXYLT2, HAGHL, HAL, HAO2, HAS3, HAVCR1, HBEGF, HCAR3, HELZ2, HES2, HGD, HHAT, HIPK2, HIVEP2, HKDC1, HMCN1, HMGCS1, HOMER1, HPGD, HRAT92, HRCT1, HSDL2, HSP90AA1, HSPA12A, HSPA1A, HSPA1B, HSPA1L, HSPA2, HSPA4L, HSPA5, HSPA6, HSPB8, HSPH1, HYAL3, ICOSLG, ID2, IDI1, IER3, IFI27, IFI44L, IFIT1, IFT80, IGF1, IGFALS, IGFBP1, IGFBP3, IGFBP6, IGFN1, IL10RA, IL12RB1, IL12RB2, IL17RB, IL18, IL36B, IL6R, IL7R, INHBE, INSIG1, IQSEC3, IRS2, ISG15, ITGA3, ITGB1BP2, ITGB6, JAKMIP3, JDP2, KCNJ11, KCNJ8, KCNQ1OT1, KIAA0125, KIAA0226L, KIAA0319, KIAA0754, KIF12, KIF14, KIF1A, KIRREL, KLF15, KLF2, KLF5, KLF9, KLHDC7B, KLHL13, KLHL3, KLHL31, KLHL32, KLK4, KRT42P, KRT7, KRT80, KRT81, KSR2, LAG3, LAMA3, LAMB3, LAMC2, LBH, LCN2, LDB2, LDLRAD4, LECT2, LEFTY1, LGALS1, LGALS4, LGALS9C, LIF, LILRA5, LINC00102, LINC00261, LINC00324, LINC00504, LINC00862, LINC00880, LINC01018, LINC01057, LINC01151, LINC01296, LINC01314, LINC01554, LOC100128494, LOC100130111, LOC100379224, LOC100506675, LOC100507195, LOC101927686, LOC101927809, LOC101927843, LOC101927973, LOC101928118, LOC101928371, LOC101928595, LOC101928837, LOC101928865, LOC101929148, LOC101929415, LOC101929427, LOC101929448, LOC101929516, LOC101929567, LOC101930114, LOC102724153, LOC104968399, LOC105371795, LOC105373051, LOC153910, LOC284344, LOC284454, LOC285629, LOC344887, LOC440300, LOC728040, LOC728752, LOC729348, LPAL2, LPCAT3, LRP8, LRRC19, LRRC24, LRRC37A3, LRRC74B, LRRIQ3, LRRN2, LTBP1, LTBP3, LYPD1, MAATS1, MAB2IL2, MAFF, MAMLD1, MANF, MAOA, MAP1B, MAP2, MAP3K8, MAP4K2, MATN2, MED31, MEGF6, MIR1282, MIR5010, MIR6087, MIR663AHG, MIR6723, MKNK1-AS1, MLK7-AS1, MLLT11, MMP12, MMP19, MMP3, MMP7, MN1, MOGAT1, MOGAT2, MOGAT3, MPPED1, MPV17L, MSC, MSMO1, MST1R, MSX1, MT1E, MT1HL1, MT1X, MTMR11, MTRNR2L3, MTRNR2L4, MVD, MXD3, MYCL, MYH4, MYL9, MYO7B, MYRIP, NAMPT, NAP1L2, NBPF10, NBPF20, NCALD, NCKAP5, NDUFA4L2, NEB, NEFM, NES, NEURL3, NEUROD1, NFATC4, NFE2L3, NLGN2, NMB, NPAS2, NPTX2, NR6A1, NRAP, NRBP2, NRG4, NRIP3, NTN1, NUAK2, NUCB2, NUDT18, NUGGC, NWD2, NYAP1, OAS1, OAS2, OAS3, OASL, OR4F13P, OSGIN1, OTUD3, OXTR, P2RY1, P4HA3, PALMD, PAPLN, PARD6G-AS1, PCAT5, PCED1B, PCK1, PDGFB, PDGFRB, PDGFRL, PDIA4, PDK4, PDLIM7, PDZK1IP1, PEG10, PELI2, PELI3, PER1, PFDN4, PFKFB3, PGD, PGF, PGM2, PHLDA1, PID1, PIK3C2G, PIK3CD, PITRM1-AS1, PKD1L2, PKHD1, PKLR, PLA2G6, PLAU, PLK2, PLPP3, PLPPR1, PLXDC1, PLXNA4, PLXND1, PNLIPRP3, PNPLA5, POC1A, POR, PPARGC1A, PPM1L, PPP1R3C, PRAMEF2, PRDM7, PREX1, PRG4, PRKD1, PROKR1, PROX1, PRR15, PRR23C, PRSS23, PSD3, PTPRB, PTPRU, RAB17, RAB39A, RAB3B, RAB3IL1, RARB, RBM11, RBP5, RCL1, RDH12, RDH5, REEP1, RGS2, RGS9, RHCG, RHOB, RHOF, RLF, RMI1, RND1, RND3, RNF122, RNVU1-19, ROBO4, ROM1, ROR1-AS1, RORA, ROS1, RPL13AP6, RPL17, RPL22L1, RPLP0P2, RPSAP9, RRAD, RTP3, RUFY4, S100A2, S100A3, S100A6, S1PR1, SALL1, SCARA3, SCART1, SCPEP1, SCUBE1, SDHAF4, SDS, SEC14L2, SEC14L4, SEMA7A, SERPINB7, SERPINB9, SESN3, SFRP4, SGK2, SH2D3C, SH3TC2, SKAP1, SLC16A13, SLC17A1, SLC17A2, SLC18A2, SLC22A10, SLC23A2, SLC25A18, SLC25A20, SLC25A30, SLC2A10, SLC2A2, SLC30A2, SLC30A8, SLC37A4, SLC38A2, SLC51B, SLC5A11, SLC6A1, SLC6A10P, SLC6A6, SLC7A11, SLC7A5, SLCO2B1, SLCO4C1, SLITRK3, SLITRK4, SMAD5-AS1, SMAD6, SMCO3, SMIM3, SMO, SMOC1, SNAI1, SNAI2, SNHG9, SNORD100, SNORD16, SNORD33, SNORD43, SNORD56, SNX22, SNX29, SOAT2, SOCS2, SOCS2-AS1, SOGA3, SORBS2, SORL1, SOX9, SPARCL1, SPINT1, SPNS2, SPOCD1, SPOCK1, SPP1, SPRR1A, SPRR3, SPTBN4, SQLE, SRPX2, SRRM3, STAMBPL1, STIP1, STK39, STOM, STOX2, SUGCT, SULT2A1, SYNGR1, SYPL2, SYTL2, SYTL5, TACSTD2, TAT, TBC1D8, TCP10L, TDO2, TEAD1, TEAD2, TENM4, TEX14, TFCP2L1, TGFB2, TGFBI, TGM3, THBS1, THRSP, TIPARP, TLE2, TMC7, TMEM135, TMEM138, TMEM156, TMEM164, TMEM25, TMEM45A, TMEM47, TMEM97, TMPPE, TMPRSS2, TMSB4X, TMTC1, TNC, TNFRSF19, TNFRSF21, TNFRSF25, TNFSF10, TNFSF11, TNFSF9, TNRC6C-AS1, TNS3, TP53INP1, TP63, TPM2, TPRG1, TPRG1-AS1, TRAF3IP3, TRAF5, TRAM2, TREML3P, TRIM10, TRIM31, TRIM55, TRPM8, TRPV3, TSC22D3, TSKU, TSLP, TSPAN15, TSPAN2, TSPAN5, TSPAN7, TSPEAR-AS1, TSPO2, TTPA, TUBA4B, TUSC3, TXNIP, TYRO3, UBALD2, UBASH3B, UBD, UBE2D1, UBR5-AS1, UBXN7, UCHL1, UGCG, UGDH-AS1, UGT1A3, UGT1A4, UGT2B4, UNC5CL, UNC93A, UPP2, UQCRHL, USP2, VEGFB, VIM, VIM-AS1, VLDLR, VLDLR-AS1, VSNL1, VWA3B, XAF1, ZBTB16, ZC3HAV1L, ZFAND2A, ZFP42, ZHX1-C8orf76, ZMYND8, ZNF485, ZNF702P, ZNF703, ZNF837, and ZXDB.

SGI-1776 was observed to significantly (p-value≤0.05) modulate expression of AASS, ABCA6, ABHD2, ABLIM3, ACAA2, ACACB, ACAD11, ACSM2A, ACSM2B, ACTA1, ACTA2, ACTG2, ADCY5, ADGRG6, ADGRV1, ADM, ADRA1A, AHNAK2, AHSG, AKAP12, AKR1B15, AKR1C8P, AKR1D1, ALAS1, ALDH1L1, ALDH3B1, ALDH8A1, ALPK2, AMACR, ANGPTL4, ANGPTL8, ANKRD33B, ANXA1, ANXA13, ANXA3, AOC4P, APOA1-AS, APOA4, APOBEC3B, AQP4, AQP7, ARHGEF26-AS1, ARID3C, ARL14, ARL4A, ASB9, ATOH8, ATP5E, ATP6V0D2, ATP6V1C2, BCAS1, BCL2, BMF, BMP2, BNIP3, BRF2, BSPRY, C10orf11, C11orf96, C19orf71, C2CD4A, C2orf91, C5orf45, C6orf222, C8orf4, CA12, CACNA1D, CACYBP, CADPS2, CAMKMT, CAPN13, CAPN8, CASKIN1, CAT, CBLB, CBLN3, CCDC152, CCL2, CCL20, CCNE2, CD109, CD163, CD36, CDC6, CDC7, CDKL1, CEACAM6, CEBPD, CECR1, CES4A, CFAP44, CHST9, CITED2, CLEC2D, CLVS1, COL10A1, COL16A1, COL7A1, COTL1, CPA4, CPB2-AS1, CPS1, CPT1A, CPVL, CREB3L3, CREB5, CRIP3, CRY2, CRYM, CSRNP3, CTDSP1, CX3CL1, CXCL1, CXCL10, CXCL11, CXCL14, CXCL6, CXCL8, CYP17A1, CYP1A1, CYP1A2, CYP1B1, CYP26A1, CYP3A4, CYP3A5, CYP7A1, CYTH1, DCDC5, DEDD2, DERL3, DHRS2, DIRAS2, DNAH5, DNAH7, DNAJB1, DNAJB4, DSG1, DUOXA2, DUSP6, DYNLL1, ECH1, ECM2, EFNA1, EFR3B, EGR2, ELOVL2, ENO2, ENO3, ENTPD7, EPB41L1, EPPK1, ERN1, ERRFI1, ERV3-1, ESR1, ETFDH, ETNPPL, EVA1B, F13B, FADS1, FADS2, FAM110A, FAM13A, FAM162A, FAM198A, FAM47E, FASN, FBXO30, FBXO41, FCAMR, FCRLA, FER1L4, FGF14, FGF2, FGFR3, FGR, FLNA, FLNC, FLRT3, FLT4, FLVCR2, FMO1, FOSB, FSIP1, FSTL3, FYB, G6PC, GADD45B, GALE, GALNT15, GAREM1, GBP4, GDF15, GHR, GNAO1, GPC6, GPCPD1, GPRIN3, HES2, HGD, HHAT, HIST1H2BG, HIVEP2, HKDC1, HMCN1, HMGCR, HMGCS2, HMGN5, HMOX1, HOGA1, HOMER1, HPGD, HSDL2, HSP90AA1, HSPA1A, HSPA1B, HSPA4L, HSPA5, HSPB1, HSPB8, HSPH1, ICOSLG, IER3, IFI35, IFIT2, IFT80, IGF1, IGFBP1, IGFBP3, IGFBP6, IL12RB1, IL17RB, IL18, IL1RL1, IL22RA1, IL6R, ILDR2, INHBB, INHBE, INSIG1, IRF5, IRS2, ITGA3, ITGA7, ITGB1BP2, ITGB6, KANK1, KCNJ11, KCNJ8, KCNK5, KCNQ1OT1, KDELR3, KIAA0895L, KIF3C, KIR3DX1, KLF2, KLF5, KLF9, KLHDC7B, KLHL13, KLHL31, KLK4, KRT42P, KRT80, KRT81, KSR2, LAMB1, LAMB3, LAMC2, LCN2, LDB2, LDHA, LECT2, LGALS1, LGALS4, LGALS8-AS1, LIF, LINC00102, LINC00261, LINC00504, LINC00862, LINC00880, LINC00933, LINC01018, LINC01057, LINC01136, LINC01151, LINC01537, LINC01587, LL22NC03-63E9.3, LMCD1, LOC100506675, LOC100507002, LOC100507487, LOC101927973, LOC101928504, LOC101928539, LOC101928858, LOC101929148, LOC101929415, LOC104968399, LOC105369486, LOC105371795, LOC105372833, LOC105376575, LOC153910, LOC440300, LOXL4, LPCAT3, LPIN2, LRP1, LRRC39, LRRIQ3, LTBP1, LUCAT1, LYPD1, MAFF, MANF, MAOA, MAP2K6, MAP3K8, MAPT, MATN2, MCM7, MEI4, MGAM, MGC12916, MIR1282, MIR663AHG, MIR6723, MIR8075, MLLT11, MMP19, MMP24, MMP3, MMP9, MOGAT2, MOGAT3, MPHOSPH6, MRO, MS4A2, MST1R, MT1E, MT1HL1, MT1JP, MT1X, MTRNR2L3, MTRNR2L4, MVD, MYCL, MYH3, MYL9, MYOM1, NBPF20, NCF2, NCKAP5, NCR3LG1, NDRG4, NDUFA4L2, NEB, NEUROD1, NEXN, NFASC, NFATC4, NIPAL4, NLRP11, NPBWR1, NPHP1, NR1D1, NRAV, NRBP2, NUCB2, OAS1, OSGIN1, PADI1, PALMD, PAM, PBX1, PCDH9, PCK1, PCSK5, PCSK9, PDIA4, PDK1, PDK4, PDLIM7, PEG10, PELI2, PELI3, PER1, PFDN4, PFKFB3, PID1, PIGR, PIP5KL1, PKHD1, PLA2G6, PLAU, PLIN2, PLIN4, PLOD1, PLPP3, PMEPA1, PNPLA5, PRG4, PROCR, PROM1, PROX1, PRR23C, PRSS23, QPCT, RAB27B, RAB3B, RAB9B, RAET1E, RAPGEF4, RARB, RASD1, RASL12, RBP5, RCL1, RDH5, REEP1, RGS9, RHOF, RIMKLA, RLF, RMI1, RND1, RND3, RNF122, ROBO4, RORA, ROS1, RPL17, RPLP0P2, RPSAP9, RRAD, RSAD2, RTP3, S100A2, S100A6, S100A9, SALL1, SCARA3, SCGN, SCN8A, SERPINB9, SESN3, SFN, SGK2, SIPA1L2, SLAMF7, SLC16A12, SLC16A13, SLC16A7, SLC17A1, SLC17A2, SLC23A2, SLC25A20, SLC25A42, SLC2A10, SLC2A2, SLC30A2, SLC44A5, SLC45A2, SLC4A8, SLC6A1, SLC7A2, SLC7A5, SLCO2B1, SLITRK4, SMAP2, SMG1P1, SMG1P3, SMIM5, SNAI2, SNHG9, SNORA25, SNORA71C, SNORD16, SNORD33, SOCS2, SOCS2-AS1, SOGA3, SORBS1, SORBS2, SORL1, SPINT1, SPNS2, SPOCD1, SPP1, SPRY1, SPRY2, SRPX2, STAMBPL1, STOX2, SUGCT, SULT2A1, SYDE2, SYTL2, SYTL5, TAAR3, TBC1D8, TBX15, TDO2, TEAD1, TEAD2, TECTB, TENM1, TEX41, TFPI2, TFRC, TGFB2, TGFBI, THRSP, TIPARP, TLR10, TLR9, TMEM45A, TMEM51, TMEM97, TMPRSS2, TMSB4X, TMTC1, TNC, TNFAIP2, TNFAIP3, TNFSF10, TNIK, TNRC6C-AS1, TNS3, TP53INP1, TP63, TRAF5, TRAM2, TRERF1, TRIM31, TRIM55, TRPM8, TSC22D3, TSKU, TSLP, TSPAN5, TSPEAR-AS1, TSPO2, TTYH3, TUBA4A, TUBA4B, UBALD2, UBXN7, UGCG, UGT2B4, UNC5CL, UNC93A, UQCRHL, USP12, VENTX, VIM, VLDLR, VWCE, YPEL2, ZBTB16, ZBTB37, ZC3HAV1L, ZFAND2A, ZNF292, ZNF295-AS1, ZNF543, ZNF608, ZNF702P, ZSWIM5, and ZXDB.

Nefazodone was observed to significantly (p-value≤0.05) modulate expression of AADAC, AASS, ABCB11, ACAD11, ACMSD, ACTA1, ACTA2, ADGRV1, ADM, ADRB2, AGRN, AHNAK2, AHRR, AHSG, AKAP12, AKR1B15, AKR1C8P, AKR1D1, ALPK2, AMACR, ANGPTL4, ANGPTL8, ANO9, ANXA13, ARC, ARG1, ARHGDIB, ARHGEF26, ARID3C, ARL14, ARL4C, ARL4D, ASB3, ATP5E, ATP6V0D2, B4GALNT1, BAG3, BAHCC1, BMP2, BNIP3, BRF2, C10orf35, C19orf71, C1orf116, C1QTNF1-AS1, C2orf82, C4orf19, C9orf72, CACYBP, CASKIN1, CBLB, CCL2, CCL5, CCNE2, CDKL1, CDKL5, CHST9, CLSTN3, CLVS2, CNN1, COL4A1, COL7A1, CPA4, CREB5, CRY2, CRYAB, CRYM, CX3CL1, CXCL8, CYB561A3, CYP1A1, CYP26A1, CYP3A4, CYP3A5, DCX, DDIT4L, DEDD2, DERL3, DNAH1, DNAH5, DNAJA4, DNAJB1, DNAJB4, DOCK10, DSG1, DUOX2, DUOXA2, DUSP13, EFNA1, EFNA3, EGOT, ELOVL2, EME2, ENO2, ENO3, EPO, EPPK1, ERN1, ESR1, EVA1A, FAM105A, FAM134B, FAM13A, FAM151A, FAM219A, FAM46C, FAM83D, FAT1, FAXC, FCAMR, FGF2, FGFR3, FKBP5, FLNA, FLVCR2, FOS, FOSB, G6PC, GADD45B, GALE, GAREM1, GCNT2, GHR, GLA, GPC6, GREM1, GREM2, HELZ2, HK2, HKDC1, HMCN1, HMGB2, HMOX1, HOMER1, HSP90AA1, HSPA12A, HSPA1A, HSPA1B, HSPA1L, HSPA4L, HSPA5, HSPA6, HSPH1, ID1, ID2, IFI44L, IFIT1, IGF1, IGFALS, IGFBP3, IL6R, IL7R, IRS2, ITGA3, ITGA7, JDP2, KANK1, KDM3A, KIAA0319, KIAA0754, KLF9, KLHDC7B, KLHL31, KRT80, KRT81, LAMB1, LAMB3, LAMC2, LAPTM5, LGALS4, LIF, LINC00261, LINC00862, LINC01314, LINC01347, LINC01554, LOC100507195, LOC101928595, LOC101929427, LOC101929448, LOC153910, LOC154761, LPCAT3, LPIN2, LTBP1, LYPD1, MAFF, MAMLD1, MAP3K8, MATN2, MDH1B, MED31, MGAM, MICALCL, MIR210HG, MLLT11, MMP1, MMP24, MOGAT2, MSC, MST1R, MT1E, MT1HL1, MT1X, MTMR11, MVD, MYCL, MYL9, MYRIP, NAP1L2, NCKAP5, NCR3LG1, NDRG1, NEB, NFASC, NPEPL1, NPTX2, NRBP2, NRG4, NUAK2, NUDT18, OAS1, OAS2, OAS3, OPRM1, OXTR, P4HA1, PAPLN, PCK1, PDF, PDGFB, PDGFRB, PDK4, PEG10, PELI3, PER1, PFDN4, PFKFB4, PHLDA1, PID1, PIGR, PLIN2, PLK2, PMAIP1, PNLIPRP3, PPFIA4, PPP1R3C, PPP1R3G, PRG4, PRKRIP1, PRSS23, PSD3, RAB27B, RAB3B, RARB, RASD1, RBP5, RCL1, RDH5, RGCC, RGS2, RLF, RMI1, RMRP, RND1, RND3, RNF122, RNVU1-19, ROM1, RPL13AP6, RPPH1, RTP3, S100A2, S100A6, S1PR1, SCARA3, SCN8A, SCPEP1, SH3GLB2, SLC17A2, SLC1A2, SLC23A2, SLC2A10, SLC2A2, SLC30A2, SLC38A2, SLC6A1, SLCO2B1, SLITRK3, SNORA25, SOCS2, SOCS2-AS1, SORL1, SOX9, SPOCD1, SPRY1, SPRY2, STIP1, SYNGR1, SYPL2, TACSTD2, TCAF2, TGFB2, TGFBI, THBS1, TIPARP, TMCC1, TMTC1, TNC, TNFAIP2, TNFRSF21, TP53INP1, TPRG1-AS1, TRIM31, TSC22D3, TTYH3, UCHL1, UNC5CL, UNC93A, VIM, VLDLR, YPEL2, ZFAND2A, ZNF485, and ZXDB.

Bms833923 was observed to significantly (p-value≤0.05) modulate expression of A1BG-AS1, ACKR4, ACSL1, ADAM19, ADCY5, AKR1B15, AKR1D1, ALAS1, ALPK2, ANGPTL8, ARHGEF26-AS1, ARHGEF3, ARL4A, ARRDC3, ATAD3C, ATOH8, ATP6V1C2, C10orf128, C12orf50, C19orf71, C1orf105, C2orf82, C5orf45, CADPS2, CAMK2B, CCDC152, CCL5, CD274, CEBPD, CLDN4, CLVS1, COL7A1, CPS1, CREB3L3, CTAGE8, CX3CL1, CXCL9, CYP1A1, CYP26A1, CYP7A1, DCX, DNAJB1, DNAJC12, DSG1, DUOX2, ECM2, EFNA1, EPHA4, EXTL3-AS1, FADS1, FAM222A, FASN, FAT1, FDFT1, FGF2, FLJ31356, FLNC, FLRT3, FOS, FOSB, FOSL1, FSTL3, G6PC, GADD45B, GBP4, GDF15, GREM1, HES4, HSPA1A, HSPA1B, HSPA5, HSPA6, HSPB8, ID2, IFI27, IFIT2, IFT80, IGSF23, IL18, IL23A, IL6R, IRF7, IVL, JAKMIP3, KCNK5, KLHL13, KRT81, LAMB3, LAMC2, LGALS4, LINC00102, LINC00261, LINC01136, LOC101929148, LOC101929516, LOC104968399, LOC105376575, LRRIQ3, LTBP1, MANF, MAP2K6, MATN2, MCM7, MIR1282, MIR6723, MKNK1-AS1, MLK7-AS1, MOG, MOGAT2, MT1HL1, MTMR11, MYH3, MYLIP, NAMPT, NR6A1, NRAP, NUCB2, OSGIN1, OXTR, PALMD, PCK1, PDGFB, PDIA4, PDK4, PFKFB3, PHLDA1, PLIN2, PLIN4, PLK2, PLOD1, PROX1, PRSS23, PTPRG-AS1, RARB, RASD1, RBP5, RND3, RPL13AP6, RSAD2, S100A2, S100A6, S100A9, SCD5, SDS, SH3TC2, SLC16A6, SLC2A10, SLC2A2, SLC6A1, SLC7A2, SLCO2B1, SNAI1, SNORD27, SNORD33, SORBS2, SOX9, THBS1, TNFAIP2, TRIM31, TRIM69, TTPA, UBD, UCN, UGT2B4, XAF1, ZFP42, ZHX1-C8orf76, and ZNF292.

Benzbromarone was observed to significantly (p-value≤0.05) modulate expression of A1BG-AS1, AADAC, ABALON, ABCA9, ABCB11, ABCC11, ABCG1, ABHD16B, ABHD2, ABLIM2, ACAA2, ACADVL, ACAT2, ACMSD, ACOT1, ACSL1, ACSL5, ACTA1, ACTA2, ACTBL2, ADCY10P1, ADGRG6, ADGRV1, ADM, ADRA1B, ADRB2, AGAP1-IT1, AGRN, AHNAK2, AHSG, AKAP12, AKR1B15, AKR1C8P, AKR1D1, ALAS1, ALDH3B1, ALDH8A1, ALPK2, AMACR, ANGPTL2, ANGPTL4, ANGPTL7, ANGPTL8, ANKRD30B, ANKRD33B, ANKRD35, ANKRD37, ANO1, ANXA13, ANXA3, APLN, APOA1-AS, APOA4, APOBEC3B, AQP4, AQP7, ARG1, ARHGDIB, ARHGEF3, ARID3C, ARL14, ARL2, ARL4A, ARL4C, ARL5B, ARRDC3, ARTS, ASB9, ASCL1, ATOH8, ATP5E, ATP6V1C2, AVPR1A, B3GNT5, B4GALNT1, BAHCC1, BCAT2, BCL2, BIRC3, BMF, BMP2, BMP8B, BSN, C10orf11, C10orf54, C19orf71, C1orf116, C1orf162, C1orf226, C2orf54, C2orf82, C4orf19, C4orf32, C4orf47, C5orf45, C9orf153, C9orf72, CA12, CA2, CACTIN-AS1, CADPS2, CAMK2G, CAMKK1, CAMKMT, CAPN13, CASKIN1, CBLB, CCBL1, CCDC103, CCDC152, CCDC85B, CCL2, CCL20, CCL5, CCNE2, CD109, CD163, CD274, CD4, CD83, CDC6, CDCA2, CDKL1, CEBPB-AS1, CEBPD, CECR1, CES4A, CFAP73, CHAD, CHST13, CHST9, CIDEC, CLDN4, CLSTN1, CLSTN3, CLVS1, CNN1, CNTLN, COL16A1, COL28A1, COL4A1, COL4A2, COX16, CPA4, CPS1, CPVL, CRACR2B, CREB5, CRIP3, CRY2, CRYAB, CRYM, CSE1L-AS1, CSPG4, CTDSP1, CTGF, CX3CL1, CXCL1, CXCL10, CXCL11, CXCL2, CXCL3, CYB561A3, CYP1A1, CYP1A2, CYP26A1, CYP2C19, CYP2C9, CYP3A4, CYP3A43, CYP3A5, CYP3A7, CYP4A22, CYP7A1, DAND5, DBH-AS1, DCX, DDIT4L, DERL3, DGKG, DGKH, DNAH1, DNAH5, DNAJB1, DNAJB4, DUOX2, DUOXA2, DUSP6, DYNLL1, ECH1, ECM2, EDARADD, EFNA1, EFNA3, EFNB2, ELOVL2, EME2, EML5, EML6, ENO2, ENO3, ENTPD7, EPAS1, EPHA4, EPHB2, EPO, EPPK1, EPSTI1, ERN1, EVA1A, EVA1B, F13B, FADS1, FADS2, FAM105A, FAM124A, FAM134B, FAM13A, FAM151A, FAM162A, FAM198A, FAM222A, FAM25A, FAM46C, FAM47E, FAM69C, FAT1, FBXO30, FBXW10, FCAMR, FDFT1, FDPS, FES, FGF13, FGF2, FGF21, FGFR3, FGL2, FKBP5, FLNA, FLNC, FLRT3, FLVCR2, FMO1, FOS, FOSB, FOSL1, FZD7, G0S2, G6PC, GADD45B, GALE, GALNT15, GBAP1, GBP4, GDF9, GEM, GHR, GJD3, GLRA2, GNAO1, GPC6, GPER1, GPR183, GPRIN3, GREM1, GREM2, GRIA3, HAL, HAO2, HAS3, HAVCR1, HELZ2, HIPK2, HIST1H3D, HIVEP2, HK2, HKDC1, HMCN1, HMGB2, HMGCR, HMGCS1, HMGCS2, HMOX1, HOGA1, HOMER1, HORMAD1, HSDL2, HSPA5, HSPB1, HSPB8, HSPH1, HTR3A, HYAL3, ICOSLG, ID1, ID2, IFI35, IFI44L, IFIT1, IFIT2, IFITM1, IFT80, IGF1, IGFALS, IGFBP1, IGSF23, IL17RB, IL18, IL22RA1, IL23A, IL36G, IL6R, ILDR2, INHBA, INHBA-AS1, INHBB, INHBE, INSIG1, INTS6-AS1, IRS2, ISG15, ITGA3, ITGA5, ITGA7, ITGB6, ITIH4-AS1, ITPKA, JAKMIP3, JMJD1C-AS1, KANK1, KCNJ8, KCNK5, KCNQ10T1, KDELR3, KDM3A, KIAA0754, KIAA1462, KIF12, KIF14, KLF15, KLF5, KLF9, KLHDC7B, KLHL31, KRT19, KRT7, KRT80, KRT81, KSR2, LAG3, LAMA3, LAMA5, LAMB1, LAMB3, LAMB4, LAMC2, LAPTM5, LDHA, LECT2, LGALS1, LGALS4, LINC00102, LINC00152, LINC00261, LINC00266-3, LINC00269, LINC00313, LINC00324, LINC00477, LINC00504, LINC00535, LINC00536, LINC00704, LINC00862, LINC01012, LINC01018, LINC01314, LINC01554, LINC01587, LOC100128494, LOC100129931, LOC100133286, LOC100506085, LOC100506321, LOC100507002, LOC100996291, LOC101927189, LOC101927587, LOC101928445, LOC101928504, LOC101928725, LOC101929415, LOC101929448, LOC101929516, LOC102724919, LOC104968399, LOC105371795, LOC105372833, LOC105373051, LOC153910, LOC154761, LOC344887, LOC440300, LOC440311, LOC644656, LOC728040, LOC729348, LOXL4, LPAL2, LPCAT3, LPIN2, LRP4, LRRC37A3, LRRN2, LTBP1, LTBP2, LTBP3, LUCAT1, LYPD1, MAFB, MAFF, MANF, MAP3K8, MAP4K2, MATN1, MATN2, MCAM, MCHR1, MCM7, METRN, MGAM, MGC12916, MIR1282, MIR210HG, MIR3661, MIR4435-2HG, MIR6087, MIR6723, MIR6753, MKNK1-AS1, MLK7-AS1, MMP24, MN1, MOGAT1, MOGAT2, MOGAT3, MPV17L, MRO, MSC, MST1R, MT1E, MT1HL1, MT1X, MTRNR2L4, MVD, MXD3, MXRA8, MYB, MYH3, MYL9, MYLIP, MYO15A, MYOF, MYOM1, MYRIP, NAV2-AS2, NBPF10, NBPF20, NCF2, NCKAP5, NCR3LG1, NDRG1, NEB, NEURL3, NEXN, NFASC, NFATC4, NIPAL4, NLRP12, NMB, NME5, NPAS2, NPB, NPEPL1, NPPB, NPR3, NPTX2, NR1D1, NR6A1, NRAP, NRARP, NRBP2, NTN1, NUAK2, NUCB1-AS1, NUCB2, NUDT18, OAS1, OAS2, OAS3, OASL, OTUD3, OXTR, P3H2, P4HA1, PABPC3, PALMD, PAM, PAPLN, PBX1, PBXIP1, PCDH9, PCK1, PDGFB, PDIA4, PDK1, PDK4, PDLIM7, PEG10, PELI2, PER1, PFKFB3, PFKFB4, PFN1P2, PHKG1, PHLDA1, PID1, PIGR, PIGZ, PIK3CD, PITRM1-AS1, PKHD1, PKLR, PKP3, PLCG1-AS1, PLIN2, PLIN4, PLK2, PLOD1, PLPP3, PMAIP1, PNLIPRP3, PNPLA5, PNPLA7, POTEE, PPFIA4, PPL, PPP1R3C, PPP1R3G, PRG4, PROCR, PROX1, PRR15, PRR23C, PRSS23, PSD3, PSORS1C3, PTRH1, PWAR5, PYCARD, RAB27B, RAB3B, RAB6B, RAB9B, RARB, RASD1, RASD2, RASIP1, RBP5, RCL1, RDH12, RDH5, RGCC, RGS2, RGS4, RHCG, RHOF, RLF, RMI1, RMRP, RND1, RND3, RNF122, RNF186, RNVU1-19, RORA, RPL13AP6, RPL17, RPL22L1, RPL23P8, RPLP0P2, RPPH1, RPSAP9, RTP3, S100A6, S1PR1, SCAMP5, SCARA3, SCD5, SCGN, SCIN, SCN8A, SCPEP1, SELM, SERPINB7, SERPINB9, SERPINE2, SESN3, SFN, SGSM1, SH3GLB2, SIPA1L2, SLC16A12, SLC16A13, SLC16A7, SLC17A1, SLC17A2, SLC18A2, SLC1A2, SLC23A2, SLC25A20, SLC25A30, SLC25A42, SLC2A10, SLC2A2, SLC45A2, SLC4A8, SLC5A11, SLC6A1, SLC6A10P, SLC7A11, SLC7A2, SLC7A5, SLC8A2, SLC9B1, SLCO2B1, SLCO4C1, SLITRK3, SMAP2, SMARCA5- AS1, SMC2-AS1, SMG1P1, SMG1P3, SMO, SMOC1, SNCG, SNHG9, SNORA19, SNORA50C, SNORD17, SNORD27, SNORD49A, SNX22, SOCS2, SOCS2-AS1, SOGA3, SORBS1, SORBS2, SORL1, SOX9, SPNS2, SPOCD1, SPP1, SPRN, SPRR1A, SPRR3, SPRY1, SPRY2, SPTBN4, SPX, SQLE, SRPX2, STAMBPL1, STIP1, STMN1, STOM, STOX2, SUCNR1, SUGCT, SULT2A1, SUSD1, SUSD3, SYDE2, SYPL2, SYTL5, TACSTD2, TAT, TBC1D8, TBX15, TBX6, TCAF2, TCP10L, TDO2, TEAD1, TEAD2, TENM1, TFCP2L1, TG, TGFB1I1, TGFB2, TGFBI, THBS1, THRSP, TIPARP, TLR10, TLR9, TMCC1, TMEM138, TMEM238, TMEM45A, TMPRSS2, TMSB4X, TNC, TNFAIP2, TNFAIP3, TNFSF10, TNFSF11, TNS2, TNS3, TOX3, TP53INP1, TPM2, TRAF3IP3, TRAF5, TRAM2, TRIM31, TRIM55, TRIM69, TRPC1, TRPV3, TSC22D3, TSLP, TSPAN15, TSPAN5, TSPO2, TTPA, TTYH3, TUBA4A, TUBA4B, TXNDC5, UBALD2, UBASH3B, UBD, UBE2D1, UBXN11, UBXN7, UCHL1, UGCG, UGDH-AS1, UGT1A4, UGT2B4, ULBP2, UNC5CL, USP12, VCAN, VENTX, VIM, VIM-AS1, VLDLR, VSIG1, VSIG4, VWCE, WTAPP1, YPEL1, YPEL2, ZC3HAV1L, ZFAND2A, ZMYND8, ZNF292, ZNF341-AS1, ZNF485, ZNF543, ZNF608, ZNF703, ZNF793, ZNF836, ZNF837, and ZXDB.

Propylthiouracil was observed to significantly (p-value≤0.05) modulate expression of A1BG-AS1, ABALON, ACAA2, APOA1-AS, ARL4A, ATAD3C, ATP5E, C18orf32, C5orf45, CASKIN1, CCDC152, CCL2, CPS1, CXCL8, CYB561A3, CYP8B1, ECH1, EIF4EBP3, ETNPPL, HAO2, ITIH4-AS1, JCHAIN, KCNQ1OT1, KLHDC7B, KLHL31, LAMB4, LGALS4, LINC00102, LOC101928504, LOC728752, MIR1282, MIR6723, MOG, MSMO1, MT1E, MT1HL1, MT1X, NMB, PFDN4, PITRM1-AS1, PTGES3L, RBP5, RND3, SAA3P, SCARNA2, SCARNA7, SCD5, SLC38A2, SLCO2B1, SNHG9, SNORD27, SNORD97, SNX22, SNX29, SOX9, TMSB4X, and VCAN.

Rapamycin was observed to significantly (p-value≤0.05) modulate expression of A1BG-AS1, ABALON, ABCA7, ABCB11, ABCG2, ABHD2, ABLIM3, ACAA2, ACACB, ACAD11, ACAT2, ACKR2, ACSL5, ACTA1, ACTG2, ADAM19, ADAMTS10, ADCY10P1, ADCY7, ADGRF2, ADGRG2, ADGRG6, ADGRV1, ADM, ADRB2, AGAP1-IT1, AHNAK2, AHSG, AKAP12, AKR1B15, AKR1C8P, AKR1D1, ALAS1, ALDH1L1, ALDH8A1, ALPK2, AMACR, ANGPTL4, ANGPTL7, ANGPTL8, ANKRD23, ANKRD36, ANO1, ANXA1, APOA1-AS, APOA4, AQP4, ARC, ARHGEF26, ARHGEF26-AS1, ARID3C, ARL14, ARL4A, ARL4C, ARL4D, ARL5B, ARRDC3, ASB14, ATAD3C, ATG9B, ATOH8, ATP5E, ATP5EP2, ATP6V1C2, AZGP1P1, B3GNT5, B4GALNT1, BAG3, BCAS1, BCAT2, BCL2, BEX4, BIK, BMF, BMP2, BMP8B, BNIP3, BRF2, BSN, BZRAP1-AS1, C10orf11, C10orf128, C10orf35, C10orf54, C11orf96, C18orf32, C1orf116, C1orf226, C1QTNF1-AS1, C2CD4A, C2orf82, C2orf91, C4orf19, C4orf32, C5orf45, C5orf49, C6orf222, C9orf152, C9orf72, CA12, CACHD1, CACYBP, CADPS2, CAMK1G, CAMK2G, CAPN13, CAPN8, CASKIN1, CAT, CBLB, CBR3, CBWD3, CCBL1, CCDC102A, CCDC152, CCDC28B, CCDC73, CCDC85B, CCL2, CCL26, CCL5, CCNE2, CCR1, CD109, CD28, CD300LB, CD83, CDC6, CDC7, CDKL1, CDKL5, CDKN1C, CEBPD, CELSR2, CENPW, CERCAM, CES4A, CES5A, CFAP47, CHST9, CITED2, CLDN2, CLDN4, CLEC2D, CLEC7A, CLSTN3, CLVS1, CNN1, CNTLN, COL1A1, COL4A1, COL4A4, COL7A1, CPA4, CPT1A, CREB3L3, CREB5, CREG2, CRIP3, CRY2, CRYAB, CRYM, CSPG4, CTGF, CUX2, CX3CL1, CXCL1, CXCL10, CXCL11, CXCL14, CXCL2, CXCL8, CYB561A3, CYP1A1, CYP1A2, CYP1B1, CYP26A1, CYP2C19, CYP2C9, CYP3A4, CYP3A43, CYP3A5, CYP7A1, CYP8B1, CYTH1, DAND5, DBH-AS1, DCX, DDIT4L, DEDD2, DEFB132, DERL3, DGKH, DNAH1, DNAH11, DNAH5, DNAH7, DNAJA4, DNAJB1, DNAJB4, DNAJC12, DNAJC22, DNM1, DOCK10, DUOXA2, DUSP13, DUSP6, DYNLL1, ECH1, ECM2, EFNA1, EFNA3, EIF4EBP3, ELOVL2, EMP2, ENO3, ENTPD1, ENTPD7, EPAS1, EPSTI1, ERC2-IT1, ERICH5, ERN1, ERRFI1, ETFDH, ETNK2, ETNPPL, EVPLL, EXTL3-AS1, F13B, F2RL2, FADS1, FADS2, FAM105A, FAM134B, FAM13A, FAM151A, FAM162A, FAM219A, FAM26F, FAM83D, FASN, FAT1, FBXO30, FBXO41, FCAMR, FCRLA, FDFT1, FDPS, FEZ1, FGF2, FKBP5, FLJ43879, FLNA, FLNC, FLVCR2, FMO1, FOS, FOSB, FOSL1, FSIP1, FSTL3, FTX, GOS2, G6PC, GADD45B, GALE, GALNT15, GAREM1, GBP4, GBPS, GCNT2, GDF7, GHR, GIPR, GLA, GLIS3, GNG4, GOLGA6L3, GOLGA7B, GP1BA, GPC6, GPM6A, GPR183, GPRASP1, GPRIN3, GREM1, GRIA3, GSG1, HAL, HBEGF, HCN2, HES4, HEYL, HIF3A, HIPK2, HIST1H4K, HIVEP2, HKDC1, HMGN5, HSD3B2, HSDL2, HSP90AA1, HSPA12A, HSPA1A, HSPA1B, HSPA1L, HSPA2, HSPA4L, HSPA6, HSPB8, HSPG2, HSPH1, ID1, ID2, IDI1, IER3, IFI35, IFI44L, IFIT1, IFT80, IGF1, IGF2BP3, IGFALS, IGFBP1, IGFBP3, IGSF23, IL12RB1, IL17RB, IL21R-AS1, IL22RA1, IL6R, IL7R, ILDR2, INHBA, INHBB, INHBE, INSIG1, IP6K3, IRF4, IRS2, ISG15, ITGA3, ITGA5, ITIH4-AS1, ITPKA, JDP2, KANK1, KCNJ11, KCNMB4, KCNQ1OT1, KDM3A, KIAA0319, KIAA1462, KIF12, KIF14, KIR3DX1, KLF2, KLF9, KLHDC7B, KLHL3, KLHL31, KLK4, KRT42P, LAMA3, LAMA5, LAMB1, LAMB3, LAMB4, LCN2, LDHA, LECT2, LGALS1, LGALS4, LGALS9, LIF, LILRA5, LINC00102, LINC00261, LINC00313, LINC00504, LINC00704, LINC00862, LINC00886, LINC01012, LINC01016, LINC01018, LINC01057, LINC01087, LINC01098, LINC01136, LINC01151, LINC01314, LINC01347, LINC01531, LINC01554, LIX1, LMCD1, LOC100128494, LOC100129931, LOC100506675, LOC100507195, LOC101927843, LOC101927973, LOC101928118, LOC101928514, LOC101928539, LOC101928597, LOC101928855, LOC101929148, LOC101929224, LOC101929227, LOC101929415, LOC101929516, LOC102724153, LOC104968399, LOC105369332, LOC153910, LOC283575, LOC283585, LOC283731, LOC349160, LOC401554, LOC440300, LOC728752, LOC729348, LOXL4, LPAL2, LPCAT3, LPIN2, LRP1, LRP8, LRRC24, LRRC74B, LRRIQ3, LRRN2, LTBP1, LYPD1, MAB2IL2, MAFB, MAFF, MAGEA10, MAMLD1, MANF, MAOA, MAP3K8, MATN2, MCM7, MED31, MEI4, METRN, MIR1282, MIR3661, MIR6087, MIR6723, MIR6732, MIR6845, MIR8075, MKNK1-AS1, MLK7-AS1, MLLT1, MLLT11, MMP12, MMP3, MMP7, MMP9, MOG, MOGAT2, MPHOSPH6, MPPED1, MS4A7, MT1E, MT1HL1, MT1X, MTRNR2L4, MXD3, MXRA8, MYCL, MYOF, MYOM1, MYRIP, NAMPT, NAP1L2, NBPF10, NBPF20, NBPF25P, NCALD, NCF2, NCKAP5, NCR3LG1, NDRG4, NEB, NEURL3, NFATC4, NIPAL4, NLGN2, NLRP6, NMB, NPR1, NPR3, NPTN-IT1, NPTX2, NR1D1, NRAP, NRBP2, NRG4, NRIP3, NTN1, NUCB1-AS1, NUCB2, NUGGC, NWD2, NYAP1, OAS1, OAS2, OAS3, OLFM2, OSGIN1, OXTR, PALMD, PAM, PAPLN, PARD6G-AS1, PBXIP1, PCA3, PCAT5, PCBP2-OT1, PCK1, PCSK5, PDGFRB, PDIA4, PDK4, PEG10, PELI2, PELI3, PER1, PFDN4, PFKFB3, PGD, PGF, PHLDA1, PID1, PIGR, PIK3CD, PIP5KL1, PITRM1-AS1, PKHD1, PLA2G6, PLCXD2, PLIN4, PLK2, PLOD1, PLPPR1, PLXND1, PNLIPRP3, POR, POU2AF1, PPARGC1A, PPL, PPM1L, PPP1R3C, PPP1R3G, PREX1, PRG4, PRICKLE2-AS3, PROX1, PRR15, PRR23C, PRSS23, PSORS1C3, PTGES3L, PTK2B, PTPRU, PWAR5, PYCARD, QPCT, RAB17, RAB27B, RAB3IL1, RASD1, RAVER2, RBP5, RCL1, RDH12, RFPL2, RGS2, RGS9, RHOB, RHOF, RLF, RMI1, RMRP, RND1, RND3, RNVU1-19, ROBO4, RORA, ROS1, RPL17, RPL22L1, RPLP0P2, RPPH1, RPSAP9, RRAD, RSAD2, RTP3, S100A2, S100A3, S100A6, S100A9, S1PR1, SALL1, SCARA3, SCD5, SCML4, SCPEP1, SCUBE1, SEC14L2, SEC14L3, SEC14L4, SERHL2, SERPINB7, SERPINB9, SERPINE2, SESN3, SGSM1, SH2D3C, SH3TC2, SIPA1L2, SLC16A13, SLC17A2, SLC1A2, SLC22A25, SLC23A2, SLC25A20, SLC29A4, SLC2A10, SLC2A2, SLC30A2, SLC30A8, SLC38A2, SLC44A5, SLC45A2, SLC4A5, SLC5A5, SLC6A1, SLC6A10P, SLC6A6, SLC7A11, SLC7A2, SLC7A5, SLCO2B1, SLITRK3, SLITRK4, SMG1P1, SMG1P3, SMO, SMOC1, SNAI1, SNHG9, SNORA71C, SNORA72, SNORD33, SNORD45B, SNX22, SNX29, SOCS2, SOCS2-AS1, SPARCL1, SPNS2, SPOCD1, SPP1, SPRR3, SPRY1, SPRY2, SPTBN4, SPX, SQLE, SRPX2, SSUH2, ST8SIA4, STAMBPL1, STC2, STIP1, STOM, STOX2, SUGCT, SULT2A1, SYDE2, SYNGR1, SYPL2, SYTL2, SYTL5, TBC1D8, TDO2, TEAD1, TEAD4, TET1, TEX41, TFCP2L1, TFPI2, TFRC, TGFBI, TGFBR3, THRSP, TIPARP, TLE2, TMC6, TMEM135, TMEM164, TMEM25, TMEM37, TMEM45A, TMEM97, TMPPE, TMPRSS2, TMSB4X, TMTC1, TNFAIP3, TNFSF10, TNRC6C-AS1, TNS2, TNS3, TP53INP1, TPRG1-AS1, TRABD2B, TRAF5, TRAM2, TRIM55, TRPM8, TSC22D3, TSKU, TSLP, TSPAN15, TTPA, TUBA4A, TXNIP, TYRO3, UBALD2, UBD, UBXN7, UCHL1, UGCG, UGDH-AS1, UGT1A4, UGT2B4, UNC5CL, UNC93A, UPP2, UQCRHL, USP12, USP2, VCAN, VENTX, VIM, VIM-AS1, VSIG1, WDR97, XAF1, YPEL2, ZBTB16, ZBTB37, ZC3H12B, ZDHHC1, ZFAND2A, ZFP42, ZHX1-C8orf76, ZMYND8, ZNF154, ZNF543, ZNF608, ZNF702P, ZNF793, ZNF827, ZNF837, and ZXDB.

BIO was observed to significantly (p-value≤0.05) modulate expression of AADAC, ABALON, ABCA10, ABCA9, ABCB11, ABCG2, ABLIM3, ACAA2, ACACB, ACAD11, ACADVL, ACAT2, ACSL5, ACSM2A, ACSM2B, ACTA1, ACTA2, ACTG2, ADAM3A, ADAMTS10, ADGRF4, ADGRG6, ADGRV1, ADM, ADRA1B, AFAP1L1, AGAP1-IT1, AHNAK2, AHSG, AK8, AKAP12, AKR1D1, ALAS1, ALDH1A3, ALDH3A1, ALOX5, ALPK2, AMACR, ANGPTL7, ANKRD29, ANKRD30BL, ANKRD33B, ANKRD36, ANO1, ANXA1, ANXA13, ANXA3, AOC4P, AP3B2, APBB3, APOA1-AS, APOBEC3B, AQP4, ARHGAP44, ARHGDIB, ARHGEF26-AS1, ARL14, ARL4C, ARL4D, ARL5B, ARTS, ASB9, ATAD3C, ATG9B, ATOH8, ATP10B, ATP5E, ATP5EP2, ATP6V1C2, AZGP1P1, B4GALNT1, BCAS1, BEST1, BIK, BIRC3, BMF, BMP2, BMP6, BRF2, BSN, C10orf11, C10orf128, C11orf96, C18orf32, C1orf105, C1QTNF1-AS1, C2orf54, C2orf82, C2orf91, C4orf19, C5orf45, C8orf4, C9orf72, CA12, CA2, CADPS2, CAMK1G, CAPN13, CAPN8, CASKIN1, CAT, CBLN3, CCBL1, CCDC152, CCDC28B, CCDC65, CCDC85B, CCL20, CD274, CDC6, CDCA2, CDKL5, CELSR3, CENPW, CEP57, CHAD, CHST13, CHST9, CITED2, CLDN4, CLEC2D, CLRN3, CLSTN1, CLSTN3, CLVS1, CNN1, CNTLN, COL16A1, COL1A1, COL28A1, COL4A1, COL7A1, CORO2B, COTL1, CPA4, CPT1A, CRACR2B, CREB3L3, CREB5, CRIP3, CRTAM, CRY2, CRYAB, CRYM, CSPG4, CUX2, CX3CL1, CXCL1, CXCL10, CXCL11, CXCL14, CXCL3, CXCL8, CXXC4, CYB561A3, CYGB, CYP17A1, CYP1A1, CYP1A2, CYP1B1, CYP1B1-AS1, CYP26A1, CYP2C19, CYP2C9, CYP3A5, CYP7A1, CYSRT1, CYTH1, DAAM2, DBH-AS1, DDIT4L, DEDD2, DGKG, DGKH, DHRS2, DNAH1, DNAH5, DNAJC12, DNAJC22, DNMT3L, DOCK10, DOK7, DPPA4, DTX1, DUSP6, DYNLL1, ECH1, ECM2, EDN1, EFNA1, EFR3B, EGFR-AS1, EML6, ENO3, ENTPD1, ENTPD7, EPAS1, EPB41L1, EPHB2, ERICH5, ERN1, ESRRG, ETNK2, ETNPPL, F2RL2, FADS1, FADS2, FAM129A, FAM134B, FAM162A, FAM198A, FAM219A, FAM25A, FAM46C, FAM47E, FAM65B, FASN, FAT1, FBXO30, FCRLA, FGF21, FGFR3, FHL3, FIBIN, FLJ31356, FLJ43879, FLNA, FLNC, FLRT3, FLVCR2, FMO1, FOSB, FOSL1, FSTL3, FTX, GOS2, G6PC, GAD1, GADD45B, GAREM1, GAS1, GBPS, GCNT2, GDF15, GEM, GHR, GLA, GLIS3, GNAZ, GOLGA7B, GPC6, GPER1, GPM6A, GPRIN3, GREM1, GREM2, GRIA3, GRMS-AS1, HAO2, HAS3, HCAR2, HCAR3, HELZ2, HES2, HES4, HGD, HHAT, HIF3A, HIPK2, HJURP, HK2, HKDC1, HMCN2, HMOX1, HOGA1, HPGD, HS3ST2, HSDL2, HSPA1A, HSPA5, HSPB1, HSPB8, ICOSLG, ID2, IFIT1, IFIT2, IFT80, IGF1, IGFALS, IGFBP1, IGFBP3, IGFBP5, IGFBP6, IGFL1, IGLL5, IGSF23, IL12RB1, IL17RB, IL18, IL1RL1, IL36B, IL6R, IL7R, INSIG1, IP6K3, IRF4, IRF5, IRF7, IRS2, ISM1, ITGA3, ITGA5, ITGA7, ITGB6, ITGB8, ITPKA, JDP2, KANK1, KANK4, KCNJ8, KCNK5, KCNMB4, KCNQ1OT1, KCP, KDELR3, KDM3A, KIAA0754, KIAA0895L, KIF12, KLF5, KLHDC7B, KLHL13, KLHL31, KLK4, KRT42P, KRT7, KRT80, KRT81, KSR2, LAG3, LAMA3, LAMA4, LAMA5, LAMB1, LAMB3, LAMB4, LAMC2, LBH, LCN2, LDLRAD4, LECT2, LGALS1, LGALS4, LGALS8-AS1, LGALS9, LGI3, LGR5, LILRA5, LINC00152, LINC00261, LINC00313, LINC00504, LINC00862, LINC00864, LINC00880, LINC00881, LINC00886, LINC00923, LINC01012, LINC01018, LINC01057, LINC01087, LINC01151, LINC01296, LINC01314, LINC01347, LIX1, LMCD1, LOC100129931, LOC100130111, LOC100506675, LOC100507002, LOC101927686, LOC101927973, LOC101928725, LOC101928855, LOC101929148, LOC101929227, LOC101929516, LOC101929631, LOC102724652, LOC104968399, LOC105369332, LOC105371795, LOC105372833, LOC283575, LOC284454, LOC285629, LOC344887, LOC349160, LOC401554, LOC644656, LOC728752, LOC729348, LOXL4, LRAT, LRCOL1, LRP8, LRRC25, LRRC37A3, LRRC74B, LRRC8C, LRRN2, LTBP1, LTBP3, LYPD1, MAFF, MAMLD1, MANF, MAOA, MAP1B, MAP2K6, MAP3K5, MATN2, MB21D2, MEGF6, MEOX2, METRN, MGAM, MGC12916, MIR1282, MIR4435-2HG, MIR6087, MIR663AHG, MIR6723, MIR8075, MLLT1, MLLT11, MMP1, MMP12, MMP19, MMP24, MOG, MPPED1, MPV17L, MST1R, MT1E, MT1HL1, MT1X, MTRNR2L4, MUC13, MXD3, MYCL, MYH3, MYL9, MYOF, MYOM1, MYPN, MYRIP, NAALADL2, NAGS, NAV3, NBPF20, NCCRP1, NCR3LG1, NDRG4, NES, NEURL3, NEUROD1, NEUROD2, NEXN, NFASC, NFATC4, NKAIN1, NMB, NPAS2, NPR1, NPTX2, NRAP, NRAV, NUAK2, NUCB2, OAS1, OAS2, OAS3, OASL, OLFM2, OSGIN1, OXTR, P4HA3, PALMD, PAM, PAPLN, PBX1, PBX1P1, PCDHA9, PCED1B, PCP4L1, PCSK5, PDGFB, PDGFRB, PDIA4, PDK4, PDLIM7, PDZK11P1, PEG10, PELI2, PFDN4, PGD, PHLDA1, PID1, PIK3CD, PITRM1-AS1, PLA2G7, PLCG1-AS1, PLCXD2, PLIN2, PLIN4, PLK2, PLOD1, PLPP3, PLXND1, PMEPA1, PNPLA7, POR, PPL, PPM1L, PPP1R3C, PRDM7, PRDM8, PROX1, PRR15, PRSS23, PTPRG-AS1, PTTG1, PWAR5, PYCARD, QPCT, RAB27B, RAB38, RAB3B, RARB, RASD1, RASD2, RASSF2, RBP5, RCL1, RGS9, RHCG, RHOF, RLF, RMRP, RND1, RND3, RNF122, RNVU1-19, ROBO4, ROR1-AS1, RORA, ROS1, RPL17, RPL22L1, RPLP0P2, RPPH1, RPSAP9, RRAD, RSAD2, RTP3, RUNX2, S100A2, S100A3, S100A6, S100A9, S1PR1, SALL1, SARDH, SCARA3, SCGN, SCPEP1, SCUBE1, SEC14L2, SEC14L3, SEC14L4, SECTM1, SEMA3E, SEMA6A, SEMA7A, SERPINB2, SERPINB9, SERPINE2, SESN3, SFN, SFRP4, SGCA, SIPA1L2, SKAP1, SLC16A12, SLC16A14, SLC17A2, SLC18A2, SLC22A10, SLC23A2, SLC25A18, SLC25A30, SLC25A42, SLC2A2, SLC30A8, SLC38A2, SLC38A4, SLC45A2, SLC4A8, SLC6A20, SLC6A6, SLC7A11, SLC7A2, SLC7A5, SLCO2B1, SLITRK4, SMAD5-AS1, SMAD6, SMAP2, SMIM3, SMOC1, SNHG9, SNORA50C, SNORD12B, SNX22, SOAT2, SOGA3, SOX9, SPARCL1, SPINT1, SPNS2, SPTBN4, SPX, STC2, STK39, STOM, SUGCT, SUSD1, SYPL2, SYTL2, SYTL4, SYTL5, TAAR3, TAC1, TAT, TBC1D8, TCAF2, TDO2, TEKT5, TFPI2, TGFB2, TGFBI, TGFBR3, TGM3, THBS1, TIPARP, TMC7, TMCC1, TMEM100, TMEM138, TMEM156, TMEM164, TMEM25, TMEM37, TMEM45A, TMEM51, TMPRSS2, TMSB4X, TNC, TNFAIP2, TNFRSF21, TNIK, TNRC6C-AS1, TNS2, TNS3, TP53INP1, TP63, TPM2, TRAM2, TREML3P, TRIM55, TSKU, TSLP, TSPAN15, TSPAN2, TSPAN5, TSPAN7, TSPO2, TTYH3, TUBA4A, UBASH3B, UBE2QL1, UBXN7, UCHL1, UGCG, UGT1A3, UGT1A4, UGT2B4, UNC5CL, UNC93A, UPP2, UQCRHL, USP12, VDR, VEGFB, VIM, VIM-AS1, VLDLR, VSNL1, VWCE, WSCD1, ZBTB37, ZC3HAV1L, ZFAND2A, ZFP42, ZMIZ1-AS1, ZNF221, ZNF292, ZNF295-AS1, ZNF608, ZNF702P, ZNF793, and ZNF827.

Adapin (doxepin) was observed to significantly (p-value≤0.05) modulate expression of ABCG2, ACAA2, ACTA2, AGRN, AHNAK2, ALPK2, ANXA1, ARL4C, ATP5E, ATP6V0D2, BSN, C19orf71, C8orf4, CA12, CASKIN1, CCL2, CD274, CDC6, COL7A1, CREB5, CX3CL1, CYBB, CYP1A1, CYP1A2, CYP3A4, CYP3A5, DERL3, DNAJC12, DSG1, EGOT, EML6, EPHA4, ETNPPL, FADS1, FADS2, FAM47E, FGF21, FLNC, HAO2, HSPA5, IFT80, IGFALS, IGFBP3, IGFBP5, IL36B, IL6R, KCP, KLHDC7B, KRT7, KRT81, KSR2, LAMC2, LECT2, LGALS1, LINC00261, LMCD1, LOC101928865, LOX, LOXL4, LRRC8C, MAMDC4, MANF, MAP6, MMP24, MT1HL1, NPEPL1, NUAK2, NUCB2, OXTR, PALMD, PDIA4, PIGR, POR, PPFIA4, PROX1, PRSS23, RAB3B, RBP5, RCL1, RRAD, S100A6, SERPINE2, SLC4A8, SYTL2, TCP10L, TDO2, TGFBI, TLR10, TMSB4X, TUBA4A, UNC5CL, and VIM.

Ibuprofen was observed to significantly (p-value≤0.05) modulate expression of AASS, ABCA9, ABCB11, ABCB9, ABLIM3, ACAA2, ACACB, ACAT2, ACSL5, ACSS2, ACTA1, ACTG2, AGRN, AHSG, AKR1B15, AMACR, ANKRD30BL, ARG1, ARHGEF26-AS1, ARL14, ARL5B, ARTS, ATAD3C, ATP5E, ATP5EP2, BIRC3, BTBD19, C11orf96, C1QTNF1-AS1, C2orf82, C2orf91, CADPS2, CBLB, CBR3, CCDC85B, CCL2, CCL20, CD274, CEBPD, CHST13, CLEC2D, CRI P3, CRTAM, CTAGE8, CXCL14, CXCL2, CYB561A3, CYP1A2, CYP2C19, CYP2C9, CYP3A5, CYP8B1, DGKH, DNAJB1, DNAJC12, DOK7, DZIP1, ECH1, EFR3B, ENO3, EPHB2, EPPK1, ERRFI1, FAM134B, FAM219A, FASN, FBXL8, FGFR3, FLRT3, G0S2, GNAO1, GPM6A, HAO2, HES2, HES4, HKDC1, HMGCR, HMGCS1, HSPA5, HSPB1, HSPB8, HYAL3, ICA1L, IER3, IFI35, IFIT1, IFIT2, IFT80, IGFALS, IGLL5, IL18, IP6K3, IRF7, ITGA7, ITGB6, ITPKA, KCNQ1OT1, KCNV1, KCP, KIAA0754, KLF2, KLF5, KRT42P, KRT7, KRT81, KSR2, LAMA5, LAMB3, LAMC2, LGALS1, LINC00102, LINC00504, LINC00886, LINC01057, LINC01296, LMCD1, LOC100130111, LOC100506675, LOC101927973, LOC101929148, LOC102724652, LOC349160, LOC728752, LOXL4, LPA, LRRC19, LTBP3, MANF, MATN2, METRN, MIR6087, MIR663A, MIR663AHG, MLLT1, MMP12, MMP3, MOG, MSC, MSMO1, MT1E, MT1HL1, MT1X, MTRNR2L4, NBPF20, NCF2, NCR3LG1, NEURL3, NEUROD1, NMB, NPHP4, NUAK2, NUDT3, OAS3, OXTR, PAPLN, PCK1, PDF, PDIA4, PDLIM7, PEG10, PFDN4, PGAM4, PID1, PIGR, PLPP3, PLXDC1, POR, PPM1L, PRG4, PRR15, PRR23C, PRSS23, PYCARD, PYGO1, RAB3B, RARB, REEP1, RHCG, RHOF, RIMKLA, RLF, RMI1, RMRP, RND3, RNVU1-19, ROR1-AS1, RPL17, RPLP0P2, RPPH1, RPSAP9, RRAD, RSAD2, S100A6, S1PR1, SDS, SEMA7A, SERPINA2, SFN, SH3TC2, SKAP1, SLC1A2, SLC22A25, SLC2A10, SLC36A2, SLC37A4, SLC7A11, SLITRK4, SNHG9, SPAG4, SPOCD1, TBC1D8, TEAD1, TGFBI, TMEM97, TMSB4X, TNC, TNFAIP3, TP53INP1, TSPAN5, TUBA4A, TXNDC5, UGCG, UGT2B4, UNC5CL, UQCRHL, USP12, VENTX, VIM, VLDLR, WBSCR27, XAF1, ZNF292, and ZNF702P.

Rosiglitazone maleate was observed to significantly (p-value≤0.05) modulate expression of AADAC, AASS, ABCC11, ACAA2, ACADVL, ACKR4, ACSL1, ACSL5, ACTBL2, ADCY5, ADGRG2, AKR1B1, AKR1C8P, ALAS1, ALDH8A1, ANGPTL4, ANKRD30BL, ANXA1, ANXA3, AQP7, ARL4A, ASPA, B3GNT5, BMF, C11orf86, C1orf116, CAT, CCL2, CCL5, CHST9, CIDEC, CNN1, COL12A1, COL15A1, COTL1, CPA4, CPT1A, CPT2, CREB3L3, CRYAB, CXCL11, CYP24A1, CYP2C19, CYP3A4, CYP3A5, CYP7A1, DHRS2, DNAH5, DRP2, DYNLL1, ECH1, EDN1, ELOVL2, EME2, ERICH5, ERV3-1, ESR1, ETFDH, ETNPPL, F13B, F2RL2, FABP3, FABP4, FAM222A, FAM69C, FGF13, FGF21, FGR, FLNA, FLRT3, GAL3ST1, GALNT15, GBAP1, GHR, GNAO1, HMGCS2, HSDL2, HSPB8, IFI44L, IFIT2, IFT80, IGF1, IGF2BP3, IGLL5, IL18, IL1RL1, IL22RA1, ILDR2, INHBB, IRF7, ITGA7, JAKMIP3, KANK1, KCNJ3, KCNJ8, KCNK3, KIAA1462, KIF3C, KLF15, KRT7, KRT80, LAMA3, LAMA4, LEFTY1, LGALS4, LINC00261, LINC00313, LINC00862, LINC01554, LMCD1, LOC100128494, LOC100507002, LOC101927755, LOC101929448, LOC145694, LPCAT3, LRRC31, MAMLD1, MAP3K8, MCAM, MGAM, MGARP, MIR6087, MIR663A, MIR663AHG, MIR663B, MIR6843, MLK7-AS1, MMP12, MMP19, MMP24, MMP3, MN1, MRGPRF, MYOF, MYRIP, NCF2, NCKAP5, NDRG1, NEB, NEURL3, NEXN, NFASC, NRAV, OAS2, OASL, OXTR, PCDH9, PCK1, PDK4, PFKFB4, PKD2L1, PKHD1, PLIN2, PLIN4, PLK2, POR, PPFIA4, PRAMEF2, PRSS23, RAPGEF4, RASD2, RDH12, RHOF, RMRP, RNF122, RNF186, RNVU1-19, RPPH1, RRAD, SCARA3, SCARNA5, SCARNA7, SECTM1, SEMA7A, SERPINB7, SGK2, SLC16A13, SLC16A7, SLC22A25, SLC25A18, SLC25A20, SLC25A42, SLC2A2, SLC6A20, SLC9B1, SNORD15B, SNORD45B, SOCS2, SOCS2-AS1, SORBS2, SPINT1, SPINT2, SPOCD1, SPX, SRPX2, STC2, SUCNR1, SULT2A1, SYNGR1, SYTL2, SYTL4, TBX15, TCP10L, TEKT5, TGFB2, TGFBR3, TH, THRSP, TMEM135, TMEM37, TNFRSF21, TNIK, TRIM31, TRIM55, TSKU, TSPAN5, TTPA, TXNIP, TYRO3, UNC93A, VLDLR, VLDLR-AS1, VSIG4, VWA3B, WSCD1, ZNF341-AS1, ZNF485, and ZNF703.

Amiodarone was observed to significantly (p-value≤0.05) modulate expression of A1BG-AS1, ABALON, ABCB11, ABCC11, ABCG2, ABHD2, ABLIM3, ACAA2, ACAD11, ACADVL, ACSL1, ACSL5, ACTA2, ADM, AKAP12, AKR1B15, AKR1D1, ALDH1L1, ALPK2, APOA1-AS, APOBEC3B, AQP4, AQP7, ARG1, ARHGEF26-AS1, ARL14, ARRDC3, ASB14, ATP5E, ATP6V1C2, BAG3, BIRC3, BMF, C18orf32, C19orf71, C2CD4A, C2orf82, C5orf45, C8orf4, CA12, CA2, CACYBP, CADPS2, CASKIN1, CBLB, CBR3, CCDC152, CCL5, CD109, CD274, CDKL1, CEBPB-AS1, CEBPD, CHST9, CLDN4, CLVS1, CNN1, CNTD1, COL7A1, CPA4, CPT1A, CREB3L3, CREB5, CRY2, CRYAB, CSPG4, CTBP1-AS, CUX2, CX3CL1, CYP1A1, CYP1A2, CYP26A1, CYP2C19, CYP2C9, CYP3A4, CYP3A5, CYP8B1, CYTH1, DAND5, DCX, DGKH, DHRS2, DNAH1, DNAJB1, DNAJB4, DYNLL1, EDARADD, EFNA1, EMP2, ENO3, EPHA4, EPO, EPPK1, ERRFI1, ETNPPL, EVA1A, FABP3, FADS1, FAM110A, FAM13A, FAM47E, FASN, FGF2, FGFR3, FKBP5, FLJ43879, FLNA, FLRT3, FLVCR2, FMO1, FOSB, FSTL3, G6PC, GADD45B, GCNT2, GDF15, GHR, GXYLT2, HBEGF, HES4, HHAT, HIPK2, HIVEP2, HKDC1, HLA-G, HSP90AA1, HSPA1A, HSPA1B, HSPA4L, HSPA5, HSPA6, HSPB1, HSPB8, HSPH1, ICOSLG, IFT80, IGF1, IGFBP1, IGFBP3, IGLL5, IL17RB, IL18, IL22RA1, IL6R, INHBA, INHBE, INSIG1, IRF7, ITGA7, ITGB6, ITIH4-AS1, JCHAIN, KCNJ8, KCNK5, KCNQ1OT1, KCP, KDELR3, KIAA0754, KLF9, KLHDC7B, KLHL13, KRT7, KRT80, KRT81, KSR2, LAMB1, LAMC2, LAPTM5, LECT2, LGALS1, LGALS4, LGALS8-AS1, LINC00261, LINC00504, LINC00862, LINC01018, LOC100128494, LOC100133286, LOC100289473, LOC101928525, LOC101929415, LOC101929516, LOC104968399, LOC105372833, LOC153910, LOC728752, LOC729348, LOXL4, LPA, LPIN2, LRRIQ3, LTBP1, LTBP3, LYPD1, MAFF, MANF, MATN2, MCM7, MIR1282, MIR6087, MIR663AHG, MIR6723, MLK7-AS1, MLLT11, MMP24, MMP3, MOGAT2, MPHOSPH6, MPV17L, MT1E, MT1HL1, MT1X, MTRNR2L4, MXD3, MXRA8, MYL9, MYLIP, NAP1L2, NBPF20, NCALD, NCF2, NCR3LG1, NEB, NR1D1, NRAP, NRAV, NRBP2, NUCB2, NUDT18, OSGIN1, OTUD3, OXTR, PABPC3, PALMD, PAPLN, PCK1, PCSK9, PDIA4, PDK4, PER1, PFN1P2, PID1, PITRM1-AS1, PLA2G6, PLIN4, PLK2, PLPP3, POR, POU2AF1, PPFIA4, PRG4, PROX1, PRR15, PRSS23, PSORS1C3, PTPRU, RAB27B, RARB, RBP5, REEP1, RHCG, RHOB, RMRP, RNVU1-19, RORA, RPL13AP6, RPL17, RPPH1, RRAD, RTP3, S100A6, S100A9, SALL1, SDS, SEMA6A, SEMA7A, SEPT4-AS1, SERPINE2, SESN3, SFN, SIPA1L2, SLC16A7, SLC22A25, SLC23A2, SLC2A2, SLC38A2, SLC4A8, SLC7A11, SLCO2B1, SMG1P1, SNHG9, SNORD17, SNORD27, SNX22, SOCS1, SOCS2, SOX9, SPOCD1, SPP1, SPRN, SPRY2, SPTBN4, SSUH2, STAMBPL1, STIP1, SUGCT, SYNGR1, SYTL2, TBC1D8, TCAF2, TGFB2, TGFBI, THBS1, THRSP, TIPARP, TMEM45A, TMPRSS2, TMSB4X, TNC, TNFAIP2, TNFAIP3, TNFRSF21, TNS2, TNS3, TP53INP1, TPRG1, TPRG1-AS1, TRAM2, TRIM31, TSKU, TSLP, TTYH3, TUBA4A, TUBA4B, TXNDC5, UBASH3B, UCHL1, UGCG, UGDH-AS1, UGT1A4, UPP2, USP12, VENTX, VIM, VIM-AS1, VWCE, ZC3HAV1L, ZNF292, ZNF485, and ZNF793.

Bms189453 was observed to significantly (p-value≤0.05) modulate expression of ABALON, ABCC11, ABCG1, ABCG2, ABLIM3, ACAA2, ACAD11, ACSS2, ADRB2, AHNAK2, AKR1D1, ALAS1, ALPK2, AMACR, ANGPTL8, ANKRD29, ANO1, APOA1-AS, ARID3C, ARRDC3, BMF, BMP8B, C5orf45, CADPS2, CASKIN1, CBLB, CBR3, CCDC152, CDKL1, CLVS1, COL4A1, CPS1, CREB3L3, CREB5, CTAGE8, CYP26A1, CYP3A5, CYP7A1, CYP8B1, DGKH, DNAH1, DNAJB1, DNAJC12, DTX1, DUSP6, EMP2, ENO3, EPO, EPPK1, ETNPPL, F13B, FADS1, FAM134B, FAM13A, FAM162A, FAM49A, FAM69C, FASN, FAT1, FCAMR, FDFT1, FDPS, FER1L4, FGF2, FGFR3, FLNA, FLRT3, FLVCR2, FSTL3, GAL3ST1, GALNT12, GBAP1, GDF15, GREM2, GYS2, HAO2, HIST3H2A, HMCN1, HMGCR, HMGCS1, HSD17B7, HSPA5, HSPA6, HSPB1, HSPB8, IFI44L, IGF1, IGFBP3, IGLL5, IL17RB, IL18, ILDR2, INHBA, INSIG1, ITGA3, KCNJ8, KCNQ1OT1, KCP, KLF2, KRT81, LAMA3, LAMC2, LDHA, LGALS1, LGALS9, LINC00102, LINC00261, LINC00504, LINC01012, LINC01587, LOC100128494, LOC100507195, LOC101929148, LOC104968399, LOC441666, LOC81691, LPA, LRAT, LRRIQ3, LTBP3, LUCAT1, LYPD1, MAMDC4, MANF, MAP2K6, MAP3K8, MCM7, MILR1, MIR663AHG, MIR6723, MLK7-AS1, MMP24, MOGAT1, MOGAT2, MSMO1, MT1HL1, NCF2, NDUFA6-AS1, NEB, NRAP, OAS1, OAS3, OTUD3, PALMD, PCDH9, PDIA4, PEG10, PFKFB3, PID1, PIGR, PKHD1, PLIN2, PLIN4, PPFIA4, PROX1, PRR15, PRSS23, PTPRU, RAB27B, RAB3B, RAET1E, RAPGEF4, RARB, RBP5, RHCG, RMRP, RRAD, RTP3, S100A6, S1PR1, SCARA3, SCN8A, SDS, SESN3, SFN, SGK2, SH2D3C, SH3TC2, SLC23A2, SLC2A10, SLC2A2, SLC4A8, SLC7A2, SLCO2B1, SNORD27, SNX22, SOAT2, SORL1, SOX9, SPAG4, STOM, TBC1D8, TCP10L, TDO2, TEKT5, TGFBI, TGFBR3, TLR10, TNFAIP2, TNFRSF21, TRIM31, TTYH3, TUBA4A, UBD, UGT2B4, USP12, USP2, VENTX, VIM, VSIG1, XAF1, and ZNF292.

Dorsomorphin was observed to significantly (p-value≤0.05) modulate expression of ABCC11, ABLIM3, ACAA2, ACACB, ACADVL, ACKR4, ACSL5, ACSM2A, ACSS2, ACTA1, ACTG2, ADGRG6, ADGRV1, ADM, AHNAK2, AKAP12, AKR1D1, ALPK2, AMACR, ANGPTL4, ANGPTL8, ANKRD30BL, ANO1, APOA1-AS, APOA4, APOBEC3B, AQP7, ARG1, ARID3C, ARL14, ASB9, ATAD3C, ATP6V1C2, AZGP1P1, BAG3, BCAT2, BMP8B, BNIP3, BRF2, C11orf96, C2CD4A, C2orf82, C5orf45, CA2, CADPS2, CAPN13, CASKIN1, CBLB, CBLN3, CCDC152, CCL2, CCL20, CD109, CDKL1, CEACAM6, CEBPD, CHST9, CLDN4, CLEC2D, CLSTN1, CLVS1, CNN1, COL12A1, COL16A1, COL4A1, CREB3L3, CREB5, CRIP3, CRY2, CRYAB, CX3CL1, CXCL1, CXCL2, CXCL3, CXCL8, CYB561A3, CYP1A1, CYP1A2, CYP1B1, CYP26A1, CYP2C19, CYP2C9, CYP3A5, CYP7A1, CYP8B1, CYTH1, DAAM2, DDIT4L, DGKH, DHRS2, DNAJB4, DOK3, DOK7, DUOX2, DUOXA2, DUSP6, EFNA1, EFNA3, EGOT, ELOVL2, ENO2, ENO3, EPAS1, EPO, EPPK1, ETNPPL, EVA1A, F13B, F2RL2, FADS1, FAM134B, FAM13A, FAM198A, FAM219A, FAM25A, FAM47E, FAM69C, FASN, FAT1, FCAMR, FDPS, FER1L4, FES, FGF2, FGL2, FKBP5, FLNA, FLNC, FLRT3, FLVCR2, FMO1, FOS, FOSB, FSTL3, G6PC, GADD45B, GALNT15, GAREM1, GBAP1, GBP4, GDF15, GHR, GLA, GLIS3, GNAO1, GREM2, HAL, HAO2, HBEGF, HES4, HK2, HSPA1A, HSPA1B, HSPB1, HSPB8, HSPH1, ICOSLG, IDI1, IFIT2, IFT80, IGF1, IGFBP1, IGFBP3, IGLL5, IGSF23, IL12RB1, IL17RB, IL18, IL22RA1, IL6R, ILDR2, INHBE, INSIG1, IRF7, ITGA3, ITGA5, ITGA7, ITGB6, KANK1, KCNE1, KCNJ8, KCNK5, KCNQ1OT1, KCNV1, KDM3A, KLF9, KLHDC7B, KLHL13, KLHL31, KRT42P, KRT7, KRT80, KRT81, KSR2, LAMA5, LAMB1, LAMC2, LDHA, LDLRAD4, LECT2, LINC00862, LINC01012, LINC01237, LOC101928725, LOC101929148, LOC104968399, LOXL4, LPA, LPCAT3, LPIN2, LRAT, LRRC37A3, LYPD1, MAMDC4, MAP2K6, MAP3K8, MATN2, MCM7, MEI4, MGC12916, MIR1282, MIR4435-2HG, MIR6087, MIR663A, MIR663AHG, MIR6723, MLK7-AS1, MMP24, MMP3, MOGAT1, MT1HL1, MT1X, MVD, MXRA8, MYH3, MYL9, NAGS, NAMPT, NCF2, NDRG1, NDUFA4L2, NEURL3, NEXN, NFASC, NPAS2, NR1D1, NRAP, NRBP2, NUAK2, NUDT3, NUGGC, OAS3, OSGIN1, OTUD3, OXTR, P4HA1, PALMD, PAPLN, PBX1, PCA3, PCDH9, PCK1, PCSK9, PDIA4, PDK1, PDK4, PEG10, PER1, PFKFB3, PFKFB4, PID1, PIGR, PITRM1-AS1, PLIN4, PLK2, PLPP3, PLXND1, POR, POTEM, PPFIA4, PPL, PPP1R3C, PRG4, PROX1, PRR15, PRSS23, PSORS1C3, QPCT, RAB17, RAB27B, RAB3B, RARB, RASD1, RBP5, RCL1, RDH5, RHOF, RLF, RMRP, RND1, RND3, RNVU1-19, RORA, RPL17, RPL22L1, RPPH1, RRAD, RTP3, S100A2, S100A6, SALL1, SCARA3, SCARNA2, SCN8A, SDS, SEC14L2, SEC14L4, SEMA7A, SEPT4-AS1, SERPINB9, SERPINE2, SESN3, SFN, SFRP4, SH3GLB2, SLC16A3, SLC16A7, SLC1A2, SLC2A2, SLC37A4, SLC45A2, SLC6A1, SLC7A11, SLC7A2, SLCO2B1, SNHG9, SNX22, SOCS2, SORL1, SOX9, SPAG4, SPP1, SPRY2, SPTBN4, STAMBPL1, STIP1, STOM, SULT2A1, SYPL2, TBC1D8, TCAF2, TDO2, TECTB, TFRC, TGFB2, TGFBI, TGFBR3, THBS1, TIPARP, TMC7, TMCC1, TMEM138, TMEM45A, TMPRSS2, TMSB4X, TNFAIP2, TNFAIP3, TNFRSF21, TNFSF10, TNS3, TP53INP1, TRIM10, TRIM31, TSC22D3, TSKU, TSPAN5, TSPEAR-AS1, TTPA, TUBA4A, TUBA4B, UCN, UGDH-AS1, UGT1A4, UGT2B4, USP12, VCAN, VIM, VIM-AS1, VWCE, XAF1, YPEL1, ZFAND2A, ZFP42, and ZNF485.

BMP2 was observed to significantly (p-value≤0.05) modulate expression of ABCA10, ABCC11, ACSL5, ADCY5, ADGRG6, ADM, AHNAK2, AHSG, ALAS1, ALDH8A1, ALPK2, AMACR, ANGPTL4, ANGPTL8, ANKRD35, ANO1, APOA1-AS, APOA4, AQP4, ARG1, ARID3C, ARRDC3, ASB9, ATAD3C, ATOH8, BCAT2, BMP2, BNIP3, CA9, CADPS2, CBLN3, CCDC152, CCL20, CD274, CDKL1, CEACAM22P, CEBPD, CHST9, CITED2, CLDN2, CLEC2D, CLSTN1, CREB3L3, CRYM, CX3CL1, CXCL2, CXCL3, CXCL6, CYB561A3, CYP1A1, CYP1A2, CYP26A1, CYP2C9, CYP3A5, CYP8B1, DOCK10, DUOX2, DUOXA2, DUSP6, EDN1, EFNA1, EFNA3, EFR3B, EGFR-AS1, ELOVL2, ENO2, ENO3, EPO, EPPK1, EVA1A, FADS1, FAM134B, FAM13A, FAM162A, FAM219A, FAM69C, FAT1, FCAMR, FDFT1, FER1L4, FGF2, FLNA, FOSB, GADD45B, GALE, GBP4, GDF15, GHR, GNAO1, GREM2, HK2, HKDC1, HMGCR, HMGCS1, HMOX1, HSD17B7, HSPA5, HSPB8, ID2, IDI1, IDO2, IFI35, IFIT2, IGFBP1, IGFBP3, IGLL5, IL18, ILDR2, INHBB, INHBE, INSIG1, ITGA3, ITGA5, ITGA7, ITGB6, KANK1, KCNK5, KCP, KDM3A, KIF12, KRT7, KRT80, KSR2, LAMA3, LAMB1, LAMC2, LAPTM5, LCN2, LDHA, LHFPL5, LINC00102, LINC00661, LINC00862, LINC01012, LINC01098, LINC01314, LINC01554, LINC01587, LOC100507389, LOC101928725, LOC104968399, LOC153910, LOC344887, LOXL4, LPA, LPCAT3, LRRC19, LRRIQ3, LYPD1, MAP1LC3B2, MAP3K8, MATN2, MCAM, MCM7, METRN, MGC12916, MIR210HG, MIR6723, MMP24, MN1, MOGAT3, MSC, MSMO1, MT1HL1, MT1X, MXD3, MYB, MYH3, NAGS, NCF2, NDRG1, NDUFA4L2, NR1D1, OAS1, OXTR, P4HA1, PAPLN, PBX1, PCK1, PCSK9, PDIA4, PDK1, PEG10, PER1, PFKFB4, PHLDA1, PID1, PIGR, PLIN4, PLPP3, PPFIA4, PPP1R3C, PPP1R3G, PROX1, PRSS23, PSORS1C3, RAB17, RAB27B, RAB3B, RARB, RASD1, RGS9, RHCG, RIMKLA, RND1, RND3, RNF122, RNF186, RPL13AP6, RPL22L1, S100A2, S100A6, S1PR1, SEPT4-AS1, SERPINE2, SESN3, SH2D3C, SLC16A3, SLC16A6, SLC1A2, SLC25A20, SLC2A10, SLC2A2, SLC37A4, SNORD27, SPAG4, SPNS2, SPOCD1, SPRY1, SPRY2, SPTBN4, TBC1D8, TBX15, TCAF2, TCP10L, TFRC, TGFB2, TH, TIPARP, TLR10, TMC7, TMCC1, TMEM138, TMEM217, TMEM45A, TNC, TNFAIP2, TNFAIP3, TNFSF10, TRIM31, TSKU, TSPAN5, TTPA, TUBA4A, UGCG, UGT2B4, UNC93A, UPP2, VCAN, VIM, VLDLR, VWA3B, VWCE, XAF1, ZFAND2A, ZNF295-AS1, and ZNF608.

Bms777607 was observed to significantly (p-value≤0.05) modulate expression of ABCB9, ABCC11, ACAD11, ACADVL, ACAT2, ACMSD, ACSL1, ACSL5, ACTA2, ADCY10P1, ADGRG6, ADM, ADRB2, AGAP1-IT1, AGRN, AHNAK2, AHRR, AKR1D1, ALDH3B1, ALPK2, AMACR, ANGPTL4, ANGPTL8, ANKRD30BL, ANKRD37, ANO1, ANXA3, AOC4P, AP3B2, APBB3, APOA4, AQP4, AQP7, ARG1, ARHGEF3, ARID3C, ARL4A, ARL4C, ARL4D, ARRDC3, ASB9, ATAD3C, ATOH8, ATP5E, ATP6V0D2, ATP6V1C2, AVPR1A, AZGP1P1, B3GNT5, BAHCC1, BCAR4, BCAT2, BMF, BMP2, BMP8B, C10orf11, C11orf96, C12orf50, C19orf71, C1orf116, C1orf162, C2orf54, C2orf82, C5orf45, CA12, CACHD1, CADPS2, CAMKK1, CAMKMT, CAPN13, CASKIN1, CBLB, CCDC103, CCDC152, CCDC183, CCL2, CCL20, CCNE2, CD109, CD274, CD4, CDC6, CDCA2, CDKL1, CDKN1C, CEBPD, CEP57, CES4A, CHST9, CITED2, CNN1, CNTD1, COL7A1, COLCA2, CPA4, CREB3L3, CREB5, CRIP3, CRY2, CRYAB, CRYM, CTGF, CX3CL1, CXCL11, CXCL2, CXCL3, CYP1A1, CYP1A2, CYP26A1, CYP2C19, CYP2C9, CYP3A4, CYP3A5, CYP4A22, CYP7A1, DCX, DDIT4L, DNAH5, DNAJB4, DNAJC12, DUSP6, ECM2, EFNA1, EFNA3, EFNB2, EGOT, ELOVL2, EML6, ENO3, EPAS1, EPHA4, EPHB2, EPO, EPPK1, ETNK2, EVA1A, F13B, FADS1, FADS2, FAM105A, FAM110A, FAM13A, FAM151A, FAM162A, FAM222A, FAM25A, FAM46C, FAM47E, FAM69C, FBXW10, FCAMR, FGF13, FGF2, FHL3, FKBP5, FLJ31104, FLNA, FMO1, FOS, FOSB, FOSL1, FSTL3, G0S2, G6PC, GADD45B, GALNT15, GDF15, GHR, GLIS3, GNAO1, GPC6, GPER1, GREM1, GREM2, GSTP1, HAL, HAO2, HAS3, HAVCR1, HCG9, HES4, HESS, HHAT, HIST1H2BG, HIST1H3D, HIVEP2, HK2, HMCN1, HMGB2, HMGCS2, HMOX1, HOMER1, HSDL2, HSPA2, HSPA5, HSPB1, HSPB8, HSPH1, ICOSLG, ID1, ID2, IDI1, IDI2, IDO2, IFI35, IFIT1, IFIT2, IFT80, IGF1, IL18, IL1RL1, IL22RA1, IL23A, IL6R, ILDR2, INHBB, INTS6-AS1, IRF7, IRS2, ITGA3, ITGA5, ITGA7, ITGB6, JAKMIP3, KANK1, KANK4, KCNE1, KCNJ8, KCNK5, KDELR3, KDF1, KDM3A, KIAA0226L, KIAA1462, KIF3C, KLF15, KLF2, KLF5, KLHDC7B, KLHL31, KLK4, KRT80, KRT81, KSR2, LAMA3, LAMA5, LAMB1, LAMB3, LAMC2, LAPTM5, LDLRAD4, LGALS1, LGALS4, LGALS8-AS1, LGALS9, LGR5, LHFPL5, LINC00261, LINC00324, LINC00862, LINC00864, LINC00880, LINC01018, LINC01314, LINC01554, LINC01587, LOC101928445, LOC101928597, LOC101929448, LOC102724450, LOC102724652, LOC105372833, LOC105376575, LOC153910, LOC344887, LOC644656, LOXL4, LPA, LRCOL1, LRRC37A3, LRRC4, LRRC66, LRRIQ3, LTBP1, LUADT1, LYPD1, MAFB, MAFF, MANF, MAP3K8, MAP7D2, MATN2, MCM7, METRN, METRNL, MGAM, MGC12916, MICALCL, MIR210HG, MIR4435-2HG, MIR663A, MIR663AHG, MIR6723, MMP24, MMP3, MOG, MPEG1, MPPED1, MRO, MSC, MST1R, MT1E, MT1HL1, MUC13, MXD3, MYCL, MYH3, MYL9, MYLIP, MYO15A, MYOF, MYOM1, MYRIP, NAMPT, NCF2, NCKAP5, NDRG1, NECAB2, NETO2, NEURL3, NFASC, NIPAL4, NLRP6, NPTX2, NR1D1, NR6A1, NTF3, NUAK2, NUCB2, NUDT18, OAS1, OAS2, OAS3, OSGIN1, OTUD3, OXTR, P4HA1, PABPC3, PAPLN, PBX1, PCK1, PDGFRB, PDIA4, PDK4, PEG10, PELI2, PER1, PFKFB3, PFKFB4, PHKG1, PHLDA1, PID1, PLIN2, PLOD1, PLPP3, PMAIP1, POR, PPFIA4, PPL, PPM1L, PPP1R3C, PPP1R3G, PROCR, PROX1, PRR15, PRSS23, PSORS1C3, RAB17, RAB27B, RAB3IL1, RARB, RASD1, RBP5, RCL1, RCN3, RDH12, RDH5, RGCC, RGS2, RHCG, RHOF, RLF, RND1, RND3, RNF122, RNF186, RPL13AP6, RPL17, RPL22L1, RSAD2, RTP3, S100A2, S100A6, SALL1, SCARA3, SCGN, SCN8A, SCPEP1, SEC14L2, SECTM1, SEMA3E, SEMA6A, SERPINE2, SFN, SFRP4, SH2D6, SH3GLB2, SIX4, SLC16A12, SLC16A13, SLC16A7, SLC17A2, SLC2A10, SLC2A2, SLC37A4, SLC38A4, SLC45A2, SLC4A8, SLC6A1, SLC6A10P, SLC6A6, SLC8A2, SLC9A5, SLC9B1, SLCO2B1, SLED1, SLITRK3, SMPD3, SNAI1, SNORA25, SNORD27, SOCS2, SOCS2-AS1, SORBS2, SOX9, SPAG4, SPINT1, SPP1, SPRY1, SPRY2, SPTBN4, STMN1, STOM, STOX2, SUCNR1, SYPL2, TAT, TBC1D8, TBX15, TCAF2, TEX26, TGFB2, TGFBI, TIPARP, TLE2, TLR10, TMC7, TMCC1, TMPRSS2, TMSB4X, TNC, TNFAIP2, TNFAIP3, TNFSF10, TOX3, TP53INP1, TRAF3IP3, TRAF5, TRIM31, TRIM69, TRPM8, TSC22D3, TSLP, TSPAN5, TTPA, TUBA4A, TUBA4B, UBALD2, UBE2D1, UCHL1, UGCG, UGT1A4, UGT2B4, USP12, VCAN, VIM, VIM-AS1, VSIG4, VWA3B, VWCE, YPEL1, YPEL2, ZFP42, ZNF292, ZNF543, ZNF703, ZNF793, ZNF827, and ZNF837.

Captopril was observed to significantly (p-value≤0.05) modulate expression of ADAMTS10, AHNAK2, AKAP12, AKR1B15, AMACR, APOBEC3B, AQP7, ARHGEF26-AS1, ARL4A, CDH12, CEBPB-AS1, CHST13, CSE1L-AS1, ELOVL2, ENO3, EPO, ETNPPL, FADS1, FDFT1, FGF2, GALNT12, GREM2, HMGCS1, ID2, IFI27, INTS6-AS1, KCNJ3, KCNJ8, LAMA5, LAMC2, LOC101929148, LOXL4, MAP1LC3B2, MMP3, MSMO1, MT1E, MT1HL1, MT1X, PCK1, PIGR, PPFIA4, PROX1, PRSS23, RARB, RASD1, RND3, SCARNA7, SESN3, SH2D3C, SLC2A2, SLC4A8, SNORD27, SNORD33, SNORD49A, SPNS2, TMEM135, TMEM37, TSKU, TUBA4A, UGT2B4, and USP12.

Atenolol was observed to significantly (p-value≤0.05) modulate expression of ABCC11, ADM, ALPK2, ARG1, C11orf96, CCL20, CEBPD, CHST9, CXCL10, CXCL2, CXCL3, CYP1A1, EFNA1, EFNA3, ELOVL2, EPO, ESR1, FADS1, FAM110A, FAM162A, FAM69C, FAT1, FLNA, GALNT12, GDF15, GNAO1, HAVCR1, HMCN1, HMOX1, HSPB8, IFT80, IGF1, INSIG1, ITGA3, ITGB6, JCHAIN, KCNJ3, KCNK5, KSR2, LAMA5, LAMC2, LHFPL5, LOC105376575, LOXL4, LPCAT3, MATN2, MIR6087, MIR663A, MIR663AHG, MMP24, MRO, MSMO1, MT1E, MT1HL1, MT1X, MYOF, OXTR, PAPLN, PCK1, PEG10, PKHD1, PROX1, RAET1E, RASD1, RMRP, RNF122, RNVU1-19, RPPH1, SCN8A, SLC16A7, SLC2A2, SLC6A1, SPRY2, TCP10L, TFRC, TGFB2, THBS1, TLR10, TMEM45A, TNC, TTPA, WBSCR27, ZFAND2A, ZHX1-C8orf76, ZNF485, and ZNF793.

Nitrofurantoin was observed to significantly (p-value≤0.05) modulate expression of A1BG-AS1, AASS, ABALON, ABCA6, ABCB11, ABCB9, ABCC11, ABCG1, ABHD2, ABLIM3, ACAA2, ACACB, ACADVL, ACAT2, ACSL1, ACSL5, ACSM2A, ACSM2B, ACSS2, ACTA1, ACTA2, ADAMTS4, ADCY1, ADCY7, ADGRF4, ADGRG2, ADGRG6, ADGRV1, ADM, ADRA1B, ADRB2, AGRN, AGTR1, AHRR, AHSG, AKAP12, AKR1B15, AKR1C8P, ALAS1, ALDH1L1, ALDH8A1, ALOX5, ALPK2, AMACR, ANGPTL2, ANO9, ANXA1, ANXA13, ANXA3, APOA1-AS, APOA4, APOBEC3B, AQP4, ARC, ARG1, ARHGEF26-AS1, ARL14, ARL4C, ARL4D, ARRDC3, ARTS, ASB9, ATAD3C, ATP5E, ATP6V0D2, ATP6V0E2-AS1, ATP6V1C2, ATP8B4, B3GNT5, BAG3, BMF, BMP8B, BNIP3, BRF2, C10orf11, C10orf54, C11orf96, C19orf71, C1orf106, C1orf116, C1QTNF1-AS1, C2CD4A, C2orf82, C4orf19, C5orf45, C5orf52, C6orf222, C9orf72, CA9, CACHD1, CACYBP, CADPS2, CAMKMT, CAPN13, CASKIN1, CASP16P, CBR3, CCBL1, CCDC152, CCL2, CCL20, CCL5, CD274, CD300LB, CDC6, CDC7, CDH12, CDH3, CDKL1, CDKL5, CDR2L, CEBPB-AS1, CEBPD, CECR1, CELSR2, CES4A, CFAP73, CHAD, CHST13, CHST3, CHST9, CIDEC, CITED2, CLDN4, CLIP2, CLRN3, CLSTN1, CLSTN3, CLVS1, CNTD1, CNTN2, COL10A1, COL16A1, COL1A1, COL7A1, COLCA2, COTL1, CPA4, CPT2, CREB3L3, CREB5, CRTAM, CRYAB, CRYM, CSPG4, CTDSP1, CX3CL1, CXCL1, CXCL2, CXCL3, CXCL8, CYB561A3, CYP1A1, CYP1B1, CYP26A1, CYP2C9, CYP3A4, CYP8B1, DDIT4L, DEFB132, DERL3, DGKG, DGKH, DNAH1, DNAH7, DNAJA4, DNAJB1, DNAJB4, DNAJC12, DOK3, DOK7, DSG1, DSG2-AS1, DUOX2, DUOXA2, DUSP13, DUSP6, DYNLL1, ECH1, ECM2, EDARADD, EDN1, EEF1A2, EFNA1, ELOVL2, EME2, EML5, EML6, EMP2, ENO2, ENO3, EPAS1, EPHB2, ERN1, ERRFI1, ESRRG, ETNPPL, EVPLL, EXTL3-AS1, FABP3, FADS1, FADS2, FAM105A, FAM124A, FAM13A, FAM162A, FAM171B, FAM222A, FAM26F, FAM47E, FAM83D, FAT3, FBXO30, FDPS, FER1L4, FGF2, FGFR3, FHL3, FLJ38576, FLNA, FLNC, FLRT3, FMO1, FOS, FOSB, FOSL1, FOXQ1, FSTL3, FTX, G6PC, GADD45B, GAL3ST1, GALNT12, GALNT15, GBP4, GBP6, GDF15, GEM, GGT3P, GHR, GLA, GNAO1, GNAZ, GPC6, GPCPD1, GPER1, GPRIN3, GREM1, HAO2, HAS3, HCAR2, HCAR3, HELZ2, HES4, HESX1, HIPK2, HIST1H2BG, HMCN1, HMGB2, HMGCR, HMGCS1, HMGCS2, HMOX1, HOMER1, HRCT1, HS1BP3-IT1, HSD17B7, HSP90AA1, HSPA1A, HSPA1L, HSPA5, HSPA6, HSPH1, HYAL3, ICA1L, ID1, ID2, IDI1, IDI2-AS1, IFI35, IFIT1, IFT80, IGF1, IGF2BP3, IGFALS, IGFBP1, IGFBP3, IGFBP5, IGLL5, IGSF23, IGSF9, IL17RB, IL18, IL1RL1, IL22RA1, IL7R, INCA1, INHBA, INHBA-AS1, INHBB, INHBE, INSIG1, INTS6-AS1, IRF7, IRS2, ISG15, ITGA3, ITGAM, ITGB1BP2, ITGB6, ITIH4-AS1, ITPKA, KANK4, KCNE1, KCNJ11, KCNJ8, KCNQ1OT1, KCP, KIAA0754, KIAA1462, KIF12, KIF14, KIF3C, KLF5, KLF9, KLHDC7B, KRT42P, KRT7, KRT80, KRT81, KSR2, LAG3, LAMA3, LAMB1, LAMB3, LAMB4, LAYN, LCN2, LDB3, LDHA, LDLRAD4, LECT2, LGALS1, LGALS4, LGALS8-AS1, LGALS9, LINC00102, LINC00261, LINC00504, LINC00536, LINC00661, LINC00704, LINC00862, LINC00880, LINC00987, LINC01018, LINC01136, LINC01151, LINC01314, LINC01347, LINC01559, LINC01587, LINGO1, LMCD1, LOC100128239, LOC100128494, LOC100129931, LOC100130111, LOC100133286, LOC100287042, LOC100507195, LOC100507389, LOC100996291, LOC101927973, LOC101928504, LOC101928525, LOC101929516, LOC104968399, LOC105369332, LOC105371795, LOC105372833, LOC105373051, LOC105376575, LOC105377763, LOC145694, LOC153910, LOC344887, LOC401554, LOC440300, LOC81691, LOX, LOXL4, LPCAT3, LPIN2, LRAT, LRP1, LRP8, LRRC24, LRRC31, LRRIQ3, LTBP1, LUCAT1, LYPD1, MACC1, MAFB, MAFF, MAMDC4, MANF, MAP1B, MAP1LC3B2, MAP2, MAP3K5, MAP3K8, MATN1-AS1, MATN2, MCHR1, MCM7, MEGF6, METRN, MGAM, MGC12916, MIR1282, MIR210HG, MIR3661, MIR4435-2HG, M1R5010, MIR6087, MIR663AHG, MIR6723, MLF1, MLLT11, MMP1, MMP12, MMP24, MMP3, MN1, MOG, MOGAT1, MOGAT2, MOGAT3, MPPED1, MPV17L, MSC, MSMO1, MST1R, MT1E, MT1HL1, MT1X, MTMR11, MTRNR2L4, MUC13, MVD, MXD3, MXRA8, MYCL, MYL9, MYOM1, MYPN, MYRIP, NAP1L2, NBPF10, NBPF20, NBPF25P, NCF2, NCKAP5, NCR3LG1, NDRG1, NDRG4, NDUFA4L2, NEB, NEURL3, NFASC, NFATC4, NIPAL4, NLGN2, NLRP6, NPAS2, NPHP4, NPR3, NPTN-IT1, NR1D1, NRAP, NRAV, NT5M, NUAK2, NUCB1-AS1, NUCB2, NUGGC, NXPH4, OAS1, OAS3, OASL, OSGIN1, OTUD3, OXTR, PABPC3, PADI1, PALMD, PAM, PAPLN, PAQR7, PBX1P1, PCA3, PCBP2-OT1, PCK1, PCSK5, PDE4B, PDGFB, PDGFRB, PDIA4, PDK1, PDK4, PDLIM7, PDZK11P1, PELI2, PELI3, PER1, PFDN4, PFKFB3, PFKFB4, PFN1P2, PGAM4, PGD, PGM2, PID1, PIK3C2G, PITRM1-AS1, PKD2L1, PKHD1, PLCG1-AS1, PLCH2, PLIN2, PLIN4, PLOD1, PLPP3, PLXNA4, PNPLA7, POLQ, POTEM, POU2AF1, PPFIA4, PPL, PPP1R27, PPP1R3C, PRG4, PRKRIP1, PRODH, PROM1, PROX1, PRR15, PRR23C, PRSS23, PSD3, PSORS1C3, PWAR5, PYCARD, RAB17, RAB3B, RAET1E, RAPGEF4, RARB, RASD1, RASL12, RBP5, RCL1, RCN3, RDH5, RGCC, RGS2, RGS4, RHOB, RLF, RMI1, RMRP, RND1, RND3, RNVU1-19, ROBO4, ROR1-AS1, ROR2, RORA, ROS1, RPL13AP3, RPL13AP6, RPL17, RPL22L1, RPPH1, RRAD, RSAD2, RTP3, S1PR1, SCARNA17, SCART1, SCD5, SCGN, SCML4, SCN8A, SDS, SERPINA2, SERPINB9, SERPINE2, SESN3, SFN, SFRP4, SGK2, SHC4, SLAMF7, SLC16A14, SLC16A3, SLC16A6, SLC17A1, SLC17A2, SLC18A2, SLC1A2, SLC22A25, SLC25A42, SLC29A4, SLC2A10, SLC2A2, SLC37A4, SLC38A2, SLC38A4, SLC45A2, SLC4A8, SLC6A1, SLC6A20, SLC7A11, SLC7A2, SLC7A5, SLCO2B1, SLED1, SLITRK3, SMARCA5-AS1, SMG1P1, SMG1P3, SMIM5, SMOC1, SNAI1, SNHG9, SNORA71C, SNORD100, SNORD12, SNORD12B, SNORD27, SNORD33, SNORD49A, SNX22, SOCS2, SORBS1, SORBS2, SORL1, SOX9, SP2-AS1, SPAG4, SPINT1, SPNS2, SPOCD1, SPP1, SPRN, SPRR3, SPRY1, SPRY2, SPTBN5, SPX, SQLE, SRPX2, SSUH2, STIP1, STOM, SUGCT, SULT2A1, SUSD1, SYDE2, SYTL2, TAC1, TBC1D8, TBX15, TDO2, TEAD1, TEAD2, TECTB, TFPI2, TFRC, TGFB2, TGFBI, THBS1, THRSP, TIPARP, TMED8, TMEM37, TMEM45A, TMEM47, TMEM97, TMSB4X, TMTC1, TNC, TNFAIP3, TNFRSF19, TNFSF10, TNFSF11, TNS2, TP53INP1, TRAF5, TREML3P, TRIM31, TRIM55, TSLP, TSPAN5, TSPAN7, TSPEAR-AS1, TTLL3, TTPA, TTYH3, TUBA1A, TUBA4A, TUBA4B, TXNIP, UBASH3B, UBE2D1, UBXN7, UCHL1, UGCG, UGDH-AS1, UGT1A4, UGT2B4, UNC5CL, UNC93A, UPP2, USP12, VDR, VEGFB, VIM, VIM-AS1, VLDLR, VWA3B, VWCE, WDR54, WTAPP1, XAF1, YJEFN3, YPEL1, ZACN, ZBTB37, ZFAND2A, ZFP42, ZMIZ1-AS1, ZMYND8, ZNF292, ZNF365, ZNF703, and ZNF837.

MK-0752 inhibitor was observed to significantly (p-value≤0.05) modulate expression of ABALON, ABCA10, ABCA6, ABCA9, ABCC11, ABCG1, ABLIM3, ACADVL, ACKR2, ACSL1, ACSL5, ACTA1, ACTA2, ADAM19, ADGRG6, ADGRV1, ADM, ADRA1A, ADRB2, AFAP1L1, AGRN, AGTR1, AHNAK2, AHSG, AKAP12, AKR1B1, AKR1C8P, ALAS1, ALDH8A1, ALPK2, AMACR, ANGPTL4, ANGPTL7, ANGPTL8, ANKRD30BL, ANKRD33B, ANKRD37, ANO1, ANXA13, APOA4, AQP4, ARG1, ARHGEF26-AS1, ARHGEF3, ARL14, ARL4A, ARL4C, ARL4D, ASB9, ATG9B, ATOH8, ATP6V0A4, ATP6V0D2, ATP6V1C2, AVPR1A, AZGP1P1, B4GALNT1, BAG3, BAHCC1, BCAS1, BIRC3, BMP2, BMP8B, BNIP3, C2orf54, C4orf32, C4orf47, C5orf45, C5orf49, C6orf222, C8orf4, C9orf72, CA12, CA2, CA9, CACHD1, CADPS2, CAMK2G, CAPN13, CAPN8, CCDC152, CCL2, CCL20, CCL5, CD274, CDCA2, CES4A, CHST13, CHST9, CITED2, CLDN2, CLDN4, CLSTN1, CLSTN3, CNN1, CNTLN, COL4A1, COL4A2, CPB2-AS1, CPS1, CREB3L3, CRY2, CRYAB, CTGF, CX3CL1, CXCL1, CXCL10, CXCL11, CXCL12, CXCL8, CYP1A1, CYP21A2, CYP24A1, CYP26A1, CYP2C19, CYP2C9, CYP3A4, CYP3A43, CYP3A5, CYP3A7, CYP3A7-CYP3A51P, CYP7A1, CYP8B1, CYTH1, DCX, DDIT4L, DEDD2, DERL3, DNAH5, DNAH7, DNAJB1, DNAJB4, DNAJC12, DOCK10, DOK3, DOK7, DSG1, DSG2-AS1, DUOX2, DUOXA2, ECH1, ECM2, EDN1, EFNA1, EFNA3, ELOVL2, EML6, ENO2, ENO3, ENPP2, EPAS1, EPHA4, EPHB2, EPPK1, EPSTI1, ESRRG, ETNPPL, EVA1A, EVPLL, F13B, F2RL2, FABP3, FADS1, FADS2, FAM13A, FAM151A, FAM198A, FAM219A, FAM49A, FAM69C, FASN, FBXO30, FBXW10, FBXW4P1, FCAMR, FER1L4, FES, FGF13, FGF19, FGFR3, FGR, FKBP5, FLNA, FLNC, FLRT3, FLVCR2, FMO1, FOS, FOSB, FOSL1, FYB, G6PC, GADD45B, GAL3ST1, GALNT12, GBP4, GCNT2, GDF15, GHR, GLA, GNAO1, GOLGA6L10, GREM2, GRIA3, HAL, HAO2, HAVCR1, HCAR2, HCAR3, HELZ2, HIST3H2A, HIVEP2, HK2, HKDC1, HMCN1, HMGB2, HMOX1, HPGD, HSD17B7, HSPA12A, HSPA1A, HSPA1B, HSPA5, HSPA6, HSPB1, HSPB8, ID2, IFI35, IFI44L, IFIT1, IFT80, IGF1, IGFALS, IGFN1, IL17RB, IL18, IL22RA1, IL23A, IL6R, IL7R, ILDR2, INSIG1, ITGA3, ITGA7, ITGAM, ITGB6, IVL, KANK1, KCNJ3, KCNJ8, KCNK5, KCP, KDM3A, KIAA0226L, KIAA0319, KLF5, KLF9, KLHL31, KRT7, KRT80, KRT81, KSR2, LAMA5, LAMB3, LAMC2, LBH, LDHA, LDLRAD4, LECT2, LEFTY1, LGALS4, LIF, LINC00261, LINC00504, LINC00535, LINC00704, LINC00862, LINC01057, LINC01151, LINC01314, LINC01554, LOC100507389, LOC101927686, LOC101928837, LOC101929415, LOC101929427, LOC101929448, LOC101929567, LOC104968399, LOC153910, LOC283731, LOC284344, LOC284454, LOC344887, LOC388942, LOC440300, LOC728040, LOXL4, LPA, LPAL2, LPIN2, LRAT, LRFN2, LRRN2, LTBP1, LUCAT1, LYPD1, MAB2IL2, MAFF, MAMLD1, MAP3K8, MCAM, MCHR1, MCM7, MED31, METRN, MGAM, MGC12916, MIR1282, MIR210HG, MIR663AHG, MIR6723, MLK7-AS1, MMP19, MMP24, MMP3, MMP7, MN1, MOGAT1, MOGAT2, MPPED1, MPV17L, MSC, MSMO1, MST1R, MT1E, MT1HL1, MTMR11, MVD, MYH3, MYH4, MYL9, MYOF, MYOM1, MYRIP, NCALD, NCKAP5, NDRG1, NDRG4, NEB, NECAB2, NEFM, NPTX2, NR1D1, NR6A1, NRAP, NUAK2, NUDT18, NUGGC, OAS1, OAS2, OAS3, OSGIN1, OTUD3, OXTR, P4HA1, PALMD, PAM, PAPLN, PCDH9, PCED1B, PCK1, PCSK9, PDE4D, PDGFB, PDGFRB, PDK1, PDK4, PDLIM7, PEG10, PELI2, PER1, PFDN4, PFKFB3, PFKFB4, PGD, PGM2, PHLDA1, PID1, PITRM1-AS1, PKD2L1, PKLR, PLAU, PLIN2, PLIN4, PLK2, PNLIPRP3, POR, PPARGC1A, PPFIA4, PPP1R3G, PRAMEF2, PRDM7, PREX1, PROM1, PROX1, PRR15, PRSS23, PSORS1C3, RAB17, RAET1E, RAPGEF4, RARB, RASD1, RASD2, RASSF2, RBP5, RCN3, RGS2, RHCG, RHOF, RIMKLA, RLF, RMI1, RMRP, RND3, RNF122, RNF186, ROR1-AS1, RORA, ROS1, RRAD, RTP3, S100A3, S100A6, SCARA3, SCGN, SCN8A, SDS, SEC14L4, SEC31B, SEMA7A, SERPINB9, SESN3, SFN, SFRP4, SGK2, SH3TC2, SHC4, SLC16A3, SLC16A5, SLC16A7, SLC17A1, SLC17A2, SLC1A2, SLC22A10, SLC25A18, SLC25A20, SLC29A4, SLC2A2, SLC38A4, SLC45A2, SLC51B, SLC6A1, SLC6A20, SLC6A6, SLC7A11, SLC7A2, SLC7A5, SLCO2B1, SLED1, SMIM3, SMOC1, SNAI1, SNX22, SOCS2, SORBS1, SORBS2, SORL1, SOX9, SPAG4, SPARCL1, SPOCK1, SPRY2, SPTBN4, SPX, SSUH2, STAMBPL1, STC2, STK39, STOM, STRA6, SUSD1, SYPL2, SYTL2, SYTL5, TBC1D8, TCAF2, TCP10L, TDO2, TEKT5, TENM2, TFCP2L1, TGFB2, TGFBI, TGFBR3, THBS1, THRSP, TIPARP, TLE6, TMC7, TMCC1, TMEM156, TMEM45A, TMEM97, TMPRSS2, TMSB4X, TNC, TNFRSF19, TNFRSF21, TNS2, TOX3, TP63, TPRG1, TRAF3IP3, TREM1, TREML3P, TRIM31, TRPM8, TRPV3, TSKU, TSPAN15, TSPAN2, TSPAN5, TSPAN7, TSPEAR-AS1, TTPA, TUBA4A, TUBA4B, UBALD2, UBASH3B, UBE2D1, UCHL1, UGCG, UGT1A3, UGT1A4, UGT1A5, UGT2B4, UNC5CL, UNC93A, USP12, VENTX, VIM, VIM-AS1, VLDLR, VLDLR-AS1, VWA3B, VWCE, XAF1, YPEL1, ZMIZ1-AS1, ZMYND8, ZNF192P1, ZNF292, ZNF341-AS1, ZNF485, ZNF703, and ZNF793.

N-Acetylpurinomycin was observed to significantly (p-value≤0.05) modulate expression of AASS, ABALON, ADGRV1, AKAP12, APOA1-AS, AQP7, ARHGEF3, ATP6V1C2, BMF, C5orf45, CASKIN1, CBR3, CCDC152, CD274, CDKL1, CLVS1, CTAGE8, CX3CL1, CYP3A5, DCLK1, DNAJC12, DSG1, DUOX2, DUOXA2, DUSP6, EDARADD, ENO3, ETNPPL, FADS1, FAM134B, FAM13A, FAM65B, FAM69C, FGF2, FLNC, FLRT3, G0S2, GDF15, GDF9, HAO2, HES4, HHAT, HKDC1, HMGCS1, HS1BP3-IT1, HSPA1B, HSPB1, ICA1L, IDI2-AS1, IGFBP1, KCNQ1OT1, KIAA1462, KLHL13, KSR2, LAMB3, LAMC2, LAPTM5, LGALS1, LGALS4, LINC00102, LINC00504, LINC01057, LINC01296, LMCD1, LOC100128494, LOC101929415, LOC104968399, LOC729348, LOXL4, LUCAT1, MAP1LC3B2, MIR1282, MIR6723, MMP3, MT1E, MT1HL1, MXD3, NCR3LG1, NRAP, PEG10, PFN1P2, PHLDA1, PID1, PITRM1-AS1, POTEM, PRKRIP1, RAB3B, RAET1E, RHOF, RLF, RMI1, RND1, RND3, RPL13AP6, RRAD, S100A6, SCD5, SLC16A6, SLC25A42, SLC2A2, SLC7A2, SNORD12B, SNORD27, SNX22, SORBS1, SORT1, SPINT1, SQLE, TBC1D8, TIPARP, TMSB4X, TNFAIP3, TRIM69, TUBA4A, TUBA4B, UBD, UGDH-AS1, UGT2B4, VENTX, and ZNF793.

LY 364947 was observed to significantly (p-value≤0.05) modulate expression of AGRN, AKAP12, ANXA3, ATAD3C, ATP6V1C2, BMF, C18orf32, C4orf19, C5orf45, CASKIN1, CAT, CCDC152, CLDN4, CLVS1, CNTD1, DNAJC12, DSG1, ELOVL2, ENO3, FAM13A, FAM49A, FAM69C, FASN, FGF2, FSTL3, GDF15, HAO2, HES4, HKDC1, HPGD, HSPB1, IFI27, IFIT2, IGFBP1, IGLL5, KRT7, LAMB4, LINC00102, LINC01012, LINC01057, LOC104968399, LRRIQ3, MAP1LC3B2, MIR6723, MT1E, MT1HL1, MXD3, NCF2, NRAP, OAS1, OAS2, OAS3, PCK1, PEG10, PIGR, PLIN2, POR, PRR23C, RAET1E, RDH12, RHOF, RND3, S100A6, SLC25A42, SLC38A2, SLC4A8, SNX22, SOX9, TLR10, TNFAIP2, TNFAIP3, TP53INP1, TRIM69, TSLP, TUBA4A, UBE2D1, UCN, UGT2B4, VIM, and XAF1.

Danazol was observed to significantly (p-value≤0.05) modulate expression of ACAA2, ACADVL, ACSM2A, ACTA1, ACTA2, ACTG2, ADGRG6, ADGRV1, ADM, AGRN, AHNAK2, AHSG, AKAP12, AKR1C8P, AKR1D1, ALDH8A1, ALPK2, AMACR, ANGPTL4, ANGPTL8, ANXA1, AQP4, ARG1, ARL14, ARL5B, ATP5E, B4GALNT1, BAG3, BCAT2, BMP2, BMP8B, BNIP3, C1orf116, C8orf4, CA12, CA2, CACYBP, CADPS2, CAPN13, CASKIN1, CBLB, CCL2, CCL20, CD109, CDKL1, CEBPD, CHST9, CLSTN3, COL7A1, CPA4, CREB5, CRY2, CUX2, CXCL3, CYBB, CYP1A1, CYP21A1P, CYP21A2, CYP26A1, CYP2C9, CYP3A4, CYP3A5, CYP7A1, DEDD2, DEFB132, DNAH7, DNAJB1, ECM2, EFNA1, EFNA3, ELOVL2, ENO2, ENO3, EPHA4, EPO, EPPK1, ERRFI1, EVA1A, F13B, FADS1, FAM134B, FAM13A, FAM219A, FAM49A, FAT1, FBXO41, FGF2, FGFR3, FLNA, FLRT3, FLVCR2, FOSB, FOSL1, FSTL3, G6PC, GADD45B, GCNT2, GDF15, GHR, GLA, GNAO1, GOLGA6L3, GPC6, HAO2, HK2, HKDC1, HMCN1, HMCN2, HMOX1, HSP90AA1, HSPA1A, HSPA1B, HSPA6, HSPB1, HSPB8, HSPH1, ID1, ID2, IFIT2, IFT80, IGF1, IGFALS, IGFBP3,1L17RB, IL18, IL6R, IL7R, INHBE, ISM1, ITGA3, ITGA7, ITGB6, KANK1, KCNJ8, KCNK5, KCP, KDM3A, KIAA0319, KLF9, KLHL31, KRT7, KRT81, LAMA3, LAMB1, LAMC2, LCN2, LDHA, LGALS4, LIF, LINC00102, LINC00261, LINC00862, LINC00880, LINC00881, LINC01314, LINC01587, LOC100507195, LOC153910, LPIN2, LRAT, LTBP1, LYPD1, MAFF, MANEA-AS1, MATN2, MGAM, MMP24, MN1, MOGAT2, MSC, MSMO1, MST1R, MT1E, MT1HL1, MTMR11, MXD3, MYL9, MYOF, MYOM1, NAMPT, NCF2, NDRG1, NEB, NES, NR1D1, NRBP2, NUAK2, OSGIN1, P4HA1, PALMD, PAPLN, PCDH9, PCK1, PDK1, PDK4, PDLIM7, PEG10, PELI3, PER1, PFKFB3, PFKFB4, PID1, PLIN4, PLK2, POTEE, PPFIA4, PPP1R3C, PPP1R3G, PROX1, PRR15, PRSS23, PSD3, PSORS1C3, PTPRU, RAB27B, RAB3B, RAET1E, RARB, RASD1, RBP5, RGS2, RHCG, RMI1, RRAD, RTP3, S100A6, SCN8A, SDS, SEC14L2, SEMA7A, SERPINB9, SERPINE2, SESN3, SHC4, SLC16A7, SLC17A1, SLC1A2, SLC25A20, SLC2A2, SLC38A4, SLC6A1, SLC7A11, SLC7A2, SLCO2B1, SMOC1, SNAI2, SOCS2, SORBS1, SORBS2, SORL1, SP2-AS1, SPNS2, SPOCD1, SPRY2, SPTBN4, STIP1, SULT2A1, SYPL2, SYTL2, TCAF2, TDO2, TGFB2, TGFBI, THBS1, TIPARP, TMCC1, TMEM135, TMEM138, TMEM45A, TMPRSS2, TMSB4X, TNC, TNFRSF21, TNS2, TP53INP1, TP63, TPRG1-AS1, TRAM2, TRPM8, TSKU, TSLP, TSPAN5, TUBA4A, TUBA4B, UBALD2, UCHL1, UCN, UNC5CL, USP12, VIM, VLDLR, VWCE, YPEL1, ZFAND2A, ZHX1-C8orf76, ZNF543, ZNF80, and ZXDB.

Sulpiride was observed to significantly (p-value≤0.05) modulate expression of ACADVL, ADGRG6, AGRN, AKAP12, ALPK2, ANGPTL4, ANKRD30BL, APOA4, APOBEC3B, BLACE, C1orf105, CASKIN1, EPPK1, FADS1, FAM162A, FAM69C, FAT1, FAXC, FDFT1, GBAP1, GDF15, GLRA2, HAO2, HES4, HSPA1A, HSPA6, ID2, IFT80, IGFBP1, IL18, IRF7, KCNV1, KLHL13, LINC00102, LINC00261, LOC101928401, LPCAT3, MAMDC4, MAP3K5, MIR663AHG, MMP24, MT1E, MT1HL1, MT1X, NCR3LG1, OAS3, P4HA1, PCK1, PMEPA1, PROX1, RAET1E, RBP5, RNVU1-19, SCAMP5, SCARNA2, SCGN, SERPINB7, SLC6A1, SLCO2B1, SNORD15B, SYNGR1, TCAF2, TLR10, TMEM164, TMEM47, TP53INP1, TTLL6, UBAP1L, VIM, ZFP42, and ZNF543.

B. RNA-Seq Result Validation

Genes ACADVL, ACAT2, ACSL1, ACSL5, AGPAT2, AGTR1, AHSG, ALAS1, AMACR, ARG1, CA12, CA2, CIDEC, COL1A1, CPS1, CPT1A, CPT2, CYP21A2, CYP2C19, CYP3A4, ECH1, ENO3, ETFDH, FABP1, G6PC, GHR, GLA, HGD, HMGCR, HMGCS2, HMOX1, HOGA1, HPGD, IFT27, LDHA, LPIN2, MOGAT2, NCF2, PCSK9, POR, RMRP, SLC25A20, SLC2A2, SLC37A4, SLC51B, SLC6A6, SULT2A1, TAT, TDO2, TMEM135, TNFSF11, and VLDLR were chosen for validation, because the genes are associated with diseases of the liver and were observed to result in an at least 2-fold change in expression in the RNA-seq pilot results. Table 31 provides a key for the reference numbers assigned to each of the 39 compounds.

The RNA of the cells was purified as described in Example 1, and gene expression changes were analyzed in duplicates using RNA-seq. The log 2 of fold changes in gene expression after administration of the compounds in Table 31 above measured by RNA-seq for disease associated genes are shown in Table 33.

TABLE 33
Log2 fold changes in gene expression

| | Compounds (MT-) | | | | | | | | | | |
|---|---|---|---|---|---|---|---|---|---|---|---|
| Gene | 51 | 723 | 487 | 505 | 361 | 277 | 835 | 193 | 610 | 117 | 182 |
| ACADVL | −0.05 | 0 | 0.02 | 0.05 | 1.38 | 0.04 | −0.02 | 0.02 | 0.05 | −0.02 | 0.07 |
| ACAT2 | 1.22 | −0.03 | −0.08 | −0.26 | −0.07 | 0.03 | 0.03 | 0.09 | 0.14 | 0 | −0.33 |
| ACSL1 | 0.39 | 0.04 | −0.01 | 0.06 | 1.55 | 0.08 | 0.01 | 0 | 0.03 | 0.01 | 0.29 |
| ACSL5 | 0.1 | 0.03 | 0.01 | −0.05 | 1.04 | 0 | 0.06 | 0.04 | 0.05 | 0 | −0.04 |
| AGPAT2 | 0.18 | −0.05 | −0.03 | −0.14 | 1.08 | −0.05 | −0.11 | 0.02 | 0.21 | 0.03 | −0.1 |
| AGTR1 | 0.12 | 0.03 | 0 | 0.25 | 0.16 | 0.04 | 0.01 | 0.01 | 0.06 | −0.04 | 0.02 |
| AHSG | −0.14 | −0.07 | 0.36 | −0.49 | −0.31 | −0.05 | 0.03 | 0.09 | 0.22 | 0.09 | −0.54 |
| ALAS1 | 0.01 | −0.01 | 0.04 | 0.21 | 0.62 | 0.01 | −0.13 | −0.06 | −0.02 | −0.12 | 0.38 |
| AMACR | −0.32 | −0.11 | −0.48 | −0.37 | −0.29 | −0.09 | −0.18 | −0.13 | −0.17 | −0.21 | −0.4 |
| ARG1 | 0.07 | 0.07 | 0.29 | 0.35 | 0.23 | 0.02 | 0.08 | 0.14 | 0.23 | 0.14 | 0.06 |
| CA12 | 0.1 | 0.29 | 0.18 | 0.16 | 0.43 | −0.02 | 0.01 | 0.03 | 0.07 | 0.03 | 0.43 |
| CA2 | −0.18 | −0.08 | −0.32 | −0.13 | 0.12 | 0.01 | −0.07 | −0.06 | 0.06 | −0.09 | −0.39 |
| CIDEC | −0.24 | 0.09 | −0.15 | −0.32 | 1.21 | −0.18 | −0.19 | −0.03 | −0.14 | −0.27 | 0.27 |
| COL1A1 | 0.55 | −0.14 | −0.12 | 0.02 | 0.1 | 0.09 | 0.03 | 0.05 | −0.02 | 0.08 | 0.37 |
| CPS1 | −0.37 | −0.04 | 0.03 | 0.02 | −0.26 | −0.03 | −0.08 | −0.06 | −0.13 | −0.1 | −0.05 |
| CPT1A | 0.02 | 0.06 | 0.05 | −0.01 | 1.43 | −0.1 | −0.04 | −0.08 | 0.06 | −0.05 | 0.21 |
| CPT2 | −0.15 | −0.09 | −0.01 | −0.13 | 1.07 | −0.03 | −0.09 | −0.09 | −0.08 | −0.15 | −0.07 |
| CYP21A2 | 0.15 | 0.08 | −0.17 | −0.18 | 0.66 | 0.16 | −0.04 | 0 | 0.18 | 0.12 | 0.33 |
| CYP2C19 | 0.1 | 0.18 | −0.03 | −0.03 | 0.61 | 0.04 | 0.07 | 0.05 | 0.02 | −0.03 | 0.22 |
| CYP3A4 | 0.03 | 0.14 | 0.11 | 0.4 | 1.02 | 0 | 0.02 | 0.06 | 0.18 | 0 | 0.46 |
| ECH1 | −0.08 | −0.04 | −0.01 | −0.22 | 1.38 | −0.03 | −0.03 | 0.02 | 0.14 | −0.05 | −0.27 |
| ENO3 | 0.1 | −0.21 | −0.37 | −0.37 | 0.25 | 0.03 | −0.26 | −0.11 | −0.24 | −0.23 | −0.12 |
| ETFDH | 0.04 | 0.05 | −0.02 | 0.09 | 1.09 | 0.06 | −0.07 | 0.05 | 0.05 | −0.05 | −0.12 |
| FABP1 | −0.04 | −0.08 | −0.02 | 0.02 | 1.58 | −0.05 | 0.02 | 0.13 | 0.21 | −0.02 | 0.04 |
| G6PC | 0.13 | 0.03 | 0.34 | 0.3 | 0.05 | −0.06 | −0.05 | −0.03 | 0.03 | 0.07 | 0.7 |
| GHR | −0.01 | −0.05 | −0.32 | −0.24 | −0.54 | −0.03 | −0.15 | −0.14 | −0.17 | −0.13 | −0.39 |
| GLA | −0.01 | 0.01 | 0.13 | 0.27 | 0.09 | 0 | −0.03 | −0.06 | 0.02 | −0.02 | 0.3 |
| HGD | −0.18 | 0 | 0.04 | 0.16 | −0.05 | 0.03 | −0.01 | −0.01 | −0.03 | −0.07 | 0.22 |
| HMGCR | 1.26 | 0.02 | 0.05 | 0.11 | 0 | 0.05 | 0.09 | 0.12 | 0.13 | 0.11 | 0.02 |
| HMGCS2 | −0.24 | −0.04 | 0.02 | 0.08 | 1.24 | −0.04 | −0.03 | 0.03 | 0.15 | −0.01 | 0.05 |
| HMOX1 | 0.21 | 0.22 | 0.31 | 0.55 | 0.6 | 0.03 | 0.09 | 0.22 | 0.38 | 0.14 | 0.24 |
| HOGA1 | −0.38 | 0.03 | 0.06 | −0.25 | −0.17 | −0.09 | −0.14 | 0.06 | 0.16 | −0.22 | −0.54 |
| HPGD | 0.35 | 0.05 | −0.09 | 0.22 | 0.32 | −0.03 | −0.01 | 0.06 | −0.03 | −0.13 | 0.08 |
| IFT27 | −0.15 | 0 | −0.02 | 0.09 | 0.38 | −0.12 | 0.07 | 0.05 | 0.25 | −0.08 | −0.11 |
| LDHA | 0.04 | −0.09 | 0.16 | 0.25 | 0.19 | 0.07 | 0.16 | 0.09 | 0.26 | 0.13 | 0.11 |
| LPIN2 | 0.14 | 0.03 | 0.19 | 0.37 | −0.11 | 0 | 0.1 | 0.05 | 0.14 | 0.04 | 0.55 |
| MOGAT2 | −0.51 | 0.01 | −0.18 | −0.51 | 0.11 | −0.11 | −0.06 | −0.08 | −0.13 | 0.01 | −0.47 |
| NCF2 | 0.19 | −0.21 | −0.34 | −0.11 | −0.62 | −0.11 | −0.24 | −0.28 | −0.18 | −0.32 | −0.01 |
| PCSK9 | 1.07 | 0.11 | 0.16 | −0.18 | 0.4 | 0 | 0.08 | 0.16 | 0.35 | 0.14 | 0.21 |
| POR | −0.04 | 0.1 | 0.02 | 0.17 | 1.62 | 0.02 | −0.11 | −0.04 | 0.04 | −0.04 | 0.36 |
| RMRP | 1.5 | −0.14 | 0.5 | 1.02 | 2.18 | 1.55 | 1.96 | 1.27 | 0.18 | −0.37 | −1.74 |
| SLC25A20 | −0.24 | −0.1 | −0.37 | −0.09 | 1.22 | −0.21 | −0.23 | −0.19 | −0.07 | −0.21 | −0.25 |
| SLC2A2 | −0.52 | −0.04 | −0.44 | −0.24 | −0.67 | −0.15 | −0.38 | −0.22 | −0.43 | −0.35 | −0.46 |
| SLC37A4 | −0.23 | −0.17 | 0.01 | −0.07 | 0.07 | 0.09 | 0.02 | 0.1 | 0.27 | 0.03 | −0.02 |
| SLC51B | 0.14 | −0.26 | 0.2 | 0.35 | 0.63 | 0.1 | −0.06 | 0.14 | −0.12 | −0.19 | −0.1 |
| SLC6A6 | 0 | 0.17 | 0 | −0.3 | 0.03 | 0.03 | −0.19 | −0.08 | −0.18 | −0.14 | −0.12 |
| SULT2A1 | −0.45 | 0.03 | −0.2 | −0.03 | 1.1 | −0.16 | −0.11 | −0.05 | −0.05 | −0.16 | −0.15 |
| TAT | 0 | −0.1 | −0.07 | 0.06 | 0.28 | −0.09 | 0 | −0.01 | 0.11 | 0.04 | −0.04 |
| TDO2 | −0.57 | −0.14 | −0.12 | 0 | −0.09 | −0.04 | −0.02 | 0.02 | −0.06 | −0.16 | 0 |
| TMEM135 | 0.09 | 0.05 | −0.17 | 0.08 | 1.02 | −0.08 | −0.19 | −0.02 | 0.03 | −0.03 | −0.03 |
| TNFSF11 | 1.09 | −0.11 | 0.03 | 0.01 | 0.02 | 0.09 | −0.05 | 0.14 | 0.2 | −0.09 | 0.03 |
| VLDLR | −0.03 | 0.01 | 0.2 | 0.28 | 1.41 | 0.13 | 0.06 | 0.06 | 0.17 | 0.14 | 0.31 |
| | Compounds (MT-) | | | | | | | | | | |
| Gene | 913 | 641 | 152 | 861 | 741 | 189 | 901 | 220 | 312 | 922 | 372 |
| ACADVL | −0.02 | 0.02 | −0.26 | 0.11 | −0.04 | −0.06 | −0.09 | 0.15 | −0.13 | −0.07 | 0.07 |
| ACAT2 | −0.22 | −0.04 | 0.12 | −0.18 | −0.05 | −0.01 | 0.06 | −0.44 | 0.08 | −0.01 | −0.48 |
| ACSL1 | −0.04 | 0.26 | −0.24 | 0.6 | 0 | −0.03 | 0.71 | 0.27 | 0.03 | −0.05 | 0.34 |
| ACSL5 | −0.08 | 0.18 | −0.21 | −0.01 | −0.05 | −0.01 | 0.65 | 0.39 | 0.05 | −0.1 | 0.19 |
| AGPAT2 | −0.19 | −0.05 | −0.73 | −0.2 | −0.11 | −0.15 | −0.46 | −0.53 | −0.22 | −0.01 | −0.51 |
| AGTR1 | −0.01 | −0.03 | 0.37 | −0.05 | −0.04 | −0.11 | 1.1 | 0.05 | −0.03 | 0.02 | 0.96 |
| AHSG | −0.37 | −0.2 | −1.08 | −0.29 | 0.03 | 0.07 | −0.57 | −0.63 | 0.04 | 0.15 | −1.23 |
| ALAS1 | 0 | 0.03 | 0.31 | −0.25 | −0.11 | −0.17 | 1.45 | 0.23 | −0.23 | 0.03 | 1.87 |
| AMACR | −0.37 | −0.22 | −0.81 | −1.4 | −0.24 | −0.4 | −0.62 | −1.05 | −0.32 | −0.16 | −0.81 |
| ARG1 | −0.04 | 0.04 | −0.72 | 1.48 | 0.01 | 0.2 | 0.63 | 0.27 | 0.16 | −0.05 | 0.25 |
| CA12 | 0.14 | 0.36 | 0.01 | 0.2 | −0.02 | 0.07 | 1.53 | 0.46 | 0.03 | 0.05 | 1.15 |
| CA2 | −0.25 | −0.22 | −0.03 | −1.05 | −0.18 | −0.25 | 0.74 | −0.32 | −0.24 | −0.12 | 0.75 |
| CIDEC | −0.35 | −0.35 | −1.22 | 1.02 | −0.24 | −0.44 | −0.36 | −0.71 | −0.44 | −0.25 | −0.04 |
| COL1A1 | 0.35 | −0.05 | −0.97 | −0.57 | 0.14 | 0.3 | −0.61 | −0.08 | 0.07 | 0 | −0.09 |
| CPS1 | −0.21 | −0.04 | −0.11 | 2.14 | −0.12 | −0.14 | −0.25 | 0.24 | −0.04 | −0.02 | −0.12 |
| CPT1A | 0.01 | 0.2 | −0.13 | 0.27 | 0.04 | −0.05 | 0.22 | 0.07 | 0.02 | 0.07 | 0.32 |
| CPT2 | −0.15 | 0 | −0.34 | 0.14 | −0.22 | −0.2 | −0.23 | −0.25 | −0.11 | −0.03 | −0.23 |
| CYP21A2 | 0.16 | 0.16 | −0.02 | −1.35 | 0.04 | 0.28 | 0.72 | 0.12 | −0.02 | 0.11 | 0.5 |
| CYP2C19 | 0.09 | 0.16 | −0.01 | 0.3 | 0.04 | 0.18 | 1.75 | 0.57 | 0.07 | 0.08 | 0.69 |
| CYP3A4 | 0.06 | 0.33 | 0.13 | 1.98 | −0.08 | 0.03 | 3.21 | 0.87 | 0.03 | −0.09 | 2.6 |
| ECH1 | −0.21 | −0.08 | −0.45 | −0.29 | −0.14 | −0.3 | −0.03 | −0.29 | −0.3 | −0.17 | −0.34 |
| ENO3 | −0.33 | −0.48 | −0.67 | −1.24 | −0.17 | −0.35 | −1.13 | −0.53 | −0.31 | −0.18 | −1.09 |
| ETFDH | −0.14 | −0.06 | −0.07 | −0.04 | −0.12 | −0.13 | −0.09 | −0.08 | −0.07 | −0.15 | −0.04 |
| FABP1 | −0.13 | −0.07 | 0.05 | −0.02 | −0.22 | −0.17 | −0.02 | 0.27 | −0.19 | −0.06 | −0.11 |
| G6PC | 0.32 | 0.5 | −0.63 | 1.02 | 0.14 | 0.13 | 0.27 | 0.22 | 0.11 | −0.1 | 0.46 |
| GHR | −0.19 | −0.18 | −0.67 | −0.42 | −0.13 | −0.32 | −0.81 | −0.6 | −0.14 | 0.01 | −0.94 |
| GLA | −0.04 | −0.06 | 0.14 | −0.25 | −0.06 | −0.1 | 0.52 | −0.11 | −0.16 | −0.02 | 1.15 |
| HGD | −0.11 | 0.04 | −0.08 | 1.23 | −0.16 | −0.16 | 0.33 | −0.08 | −0.19 | 0 | 0.41 |
| HMGCR | 0.02 | 0.08 | 0.47 | 0 | 0.05 | 0.12 | 0.2 | 0.17 | 0.13 | −0.02 | 0.12 |
| HMGCS2 | 0.05 | 0.03 | 0.26 | 0.07 | −0.03 | 0.01 | 0.11 | 0.8 | 0.01 | 0.03 | 0 |
| HMOX1 | 0.1 | −0.06 | 1.66 | −0.44 | 0.05 | 0.29 | 0.35 | 0.76 | 0.08 | −0.08 | 0.12 |
| HOGA1 | −0.16 | 0.1 | 0.05 | 1 | 0 | −0.15 | 0.19 | −0.42 | −0.11 | −0.12 | 0.01 |
| HPGD | −0.1 | −0.08 | −0.09 | 2.07 | −0.06 | −0.17 | −0.52 | 0.05 | 0 | −0.24 | −0.59 |
| IFT27 | −0.16 | −0.14 | −0.34 | −0.3 | −0.01 | −0.16 | −0.16 | −1.05 | −0.34 | 0.03 | −0.1 |
| LDHA | −0.03 | −0.06 | −1.49 | 0.39 | 0.08 | 0.2 | 0.19 | −0.05 | 0.22 | −0.04 | −0.34 |
| LPIN2 | 0.16 | 0.43 | −0.31 | 1.93 | 0.17 | 0.2 | −0.26 | 0.5 | 0.21 | −0.03 | 0.15 |
| MOGAT2 | −0.13 | −0.11 | −0.72 | 0.04 | 0.13 | 0 | −0.35 | −0.17 | 0 | −0.03 | −1.14 |
| NCF2 | −0.27 | −0.56 | −0.5 | −1.71 | −0.19 | −0.26 | 0.24 | −0.94 | −0.26 | −0.11 | 0.1 |
| PCSK9 | 0.2 | 0.24 | −0.02 | 0.11 | 0.17 | 0.28 | 0.59 | 0.04 | 0.12 | 0.07 | −0.37 |
| POR | −0.07 | 0.16 | −0.17 | 0.87 | −0.14 | −0.05 | 1.13 | −0.03 | −0.28 | 0.01 | 1.44 |
| RMRP | −0.98 | −1.59 | −0.63 | −1.43 | −1.45 | 1.05 | −1.29 | −1.74 | −2.27 | −0.73 | 0.11 |
| SLC25A20 | −0.42 | −0.11 | −0.29 | −0.08 | −0.31 | −0.37 | −0.46 | −0.36 | −0.38 | −0.02 | −0.42 |
| SLC2A2 | −0.45 | −0.31 | −0.55 | 0.41 | −0.22 | −0.43 | −0.99 | −0.74 | −0.24 | −0.18 | −0.85 |
| SLC37A4 | −0.01 | 0.02 | −0.8 | 1.16 | 0.09 | 0.15 | −0.06 | −0.04 | 0.12 | 0.13 | −0.4 |
| SLC51B | −0.02 | 0.36 | −0.22 | 0.08 | −0.08 | −0.14 | 1.02 | 0.4 | −0.22 | −0.2 | 0.96 |
| SLC6A6 | −0.11 | 0.09 | −0.34 | −0.8 | −0.35 | −0.18 | 1.28 | −0.44 | −0.26 | −0.09 | 0.47 |
| SULT2A1 | −0.3 | −0.12 | −0.25 | 0.39 | −0.18 | −0.25 | 0.38 | −0.45 | −0.19 | −0.18 | 0.21 |
| TAT | 0.01 | −0.15 | −0.27 | 2.37 | 0.09 | 0.1 | −0.3 | 0.32 | 0.05 | −0.08 | −0.42 |
| TDO2 | −0.31 | −0.03 | −0.72 | 1.05 | −0.14 | −0.29 | −0.42 | −0.24 | −0.07 | −0.16 | −0.71 |
| TMEM135 | −0.17 | −0.02 | −0.15 | −0.08 | −0.16 | −0.16 | 0.14 | −0.13 | 0.04 | −0.15 | −0.35 |
| TNFSF11 | 0.1 | −0.15 | 0.59 | −0.27 | 0.04 | 0.02 | 0.06 | 0.43 | 0.31 | 0.01 | −0.5 |
| VLDLR | 0.05 | 0.06 | −0.53 | 0.55 | 0.09 | 0.16 | 1.69 | 0.54 | 0.14 | −0.07 | 1.23 |

In the validation, p-values for each change in expression were 0.05 and were all considered statistically significant. Bms833923 (MT-636), dmPGE2 (MT-937), N-Acetylpurinomycin (MT-993), and LY 364947 (MT-740) were not observed to cause a greater than 2-fold change in expression in any gene.

### Example 3. Validating Gene Expression Results

qRT-PCR was performed on samples of primary human hepatocytes from a second donor. The hepatocytes were stimulated with simvastatin, rosiglitazone maleate, sulpiride, imatinib, amiodarone, nitrofurantoin, prednisone, penicillamine (D-), rifampicin, isoniazid, ritonavir, ibuprofen, propylthiouracil, BIO, xav939, or MK-0752, which were observed to cause at least a 2-fold change in the expression level of at least one DAG in Table 29 for 58 genes associated with liver disease. Benzbromarone was observed to be cytotoxic, and was not further analyzed after the pilot RNA-seq. The 58 liver disease associated genes analyzed were ACADVL, ACAT2, ACSL1, ACSL5, AGPAT2, AGTR1, AHSG, ALAS1, AMACR, ANGPTL2, ARG1, CA12, CA2, CIDEC, COL1A1, CPS1, CPT1A, CPT2, CYP21A2, CYP2C19, CYP3A4, CYP7A1, ECH1, ENO3, ETFDH, FABP1, FABP6, G6PC, GHR, GLA, GYS2, HGD, HMGCR, HMGCS2, HMOX1, HOGA1, HPGD, IFT27, LDHA, LPIN2, MOGAT2, NCF2, PCSK9, POR, RMRP, SLC25A20, SLC2A2, SLC37A4, SLC51B, SLC6A20, SLC6A6, SOAT2, SULT2A1, TAT, TDO2, TMEM135, TNFSF11, and VLDLR as shown in Example 2.

Table 34 compares results of qRT-PCR for genes that were observed to have an at least 2-fold change in expression in the RNA-seq results in Table 33.

TABLE 34
Comparison of fold change in gene expression measured by RNA-seq and qRT-PCR

| Cmpd | | | RNA-seq fold Δ | Expected fold | Cmpd | | | RNA-seq fold Δ | Expected fold |
|---|---|---|---|---|---|---|---|---|---|
| (MT-) | Gene | Fold Δ | (Log2) | Δ | (MT-) | Gene | Fold Δ | (Log2) | A |
| 51 | ACAT2 | 2.87 | 1.22 | 2.33 | 861 | HPGD | 1.28 | 2.07 | 4.20 |
| 51 | CYP7A1 | 0.85 | −1.42 | 0.37 | 861 | NCF2 | 0.76 | −1.71 | 0.31 |
| 51 | HMGCR | 4.02 | 1.26 | 2.39 | 861 | RMRP | 0.79 | −1.43 | 0.37 |
| 51 | PCSK9 | 5.76 | 1.07 | 2.10 | 861 | SLC37A4 | 1.99 | 1.16 | 2.23 |
| 51 | TNFS11 | 1.01 | 1.09 | 2.13 | 861 | TAT | 9.55 | 2.37 | 5.17 |
| 361 | ACADVL | 2.05 | 1.38 | 2.60 | 861 | TDO2 | 2.66 | 1.05 | 2.07 |
| 361 | ACSL1 | 4.06 | 1.55 | 2.93 | 861 | ANGPTL2 | 1.00 | −1.22 | 0.43 |
| 361 | CIDEC | 0.77 | 1.21 | 2.31 | 861 | LPIN2 | 1.45 | 1.93 | 3.81 |
| 361 | CPT1A | 2.19 | 1.43 | 2.69 | 741 | RMRP | 0.71 | −1.45 | 0.37 |
| 361 | CPT2 | 13.5 | 1.07 | 2.10 | 901 | AGTR1 | 0.78 | 1.10 | 2.14 |
| 361 | CYP3A4 | 2.13 | 1.02 | 2.03 | 901 | ALAS1 | 1.23 | 1.45 | 2.73 |
| 361 | ECH1 | 1.41 | 1.38 | 2.60 | 901 | CA12 | 1.44 | 1.53 | 2.89 |
| 361 | ETFDH | 1.64 | 1.09 | 2.13 | 901 | CYP2C19 | 0.77 | 1.75 | 3.36 |
| 361 | FABP1 | 1.92 | 1.58 | 2.99 | 901 | CYP3A4 | 2.83 | 3.21 | 9.25 |
| 361 | HMGCS2 | 1.14 | 1.24 | 2.36 | 901 | CYP7A1 | 0.32 | −1.27 | 0.41 |
| 361 | POR | 3.17 | 1.62 | 3.07 | 901 | ENO3 | 0.96 | −1.13 | 0.46 |
| 361 | SLC25A20 | 1.17 | 1.22 | 2.33 | 901 | POR | 1.11 | 1.13 | 2.19 |
| 361 | SULT2A1 | 5.09 | 1.10 | 2.14 | 901 | SLC51B | 3.28 | 1.02 | 2.03 |
| 361 | TMEM135 | 2.06 | 1.02 | 2.03 | 901 | SLC6A20 | 0.89 | 1.03 | 2.04 |
| 361 | VLDLR | 4.12 | 1.41 | 2.66 | 901 | SLC6A6 | 0.91 | 1.28 | 2.43 |
| 361 | AGPAT2 | 0.23 | 1.08 | 2.11 | 901 | VLDLR | 1.54 | 1.69 | 3.23 |
| 361 | ACSL5 | 1.70 | 1.04 | 2.06 | 312 | RMRP | 0.43 | −2.27 | 0.21 |
| 277 | RMRP | 2.55 | 1.55 | 2.93 | 372 | ALAS1 | 1.53 | 1.87 | 3.66 |
| 277 | CYP7A1 | 0.65 | −1.24 | 0.42 | 372 | CA12 | 2.00 | 1.15 | 2.22 |
| 277 | RMRP | 2.35 | −1.74 | 0.30 | 372 | CYP3A4 | 2.08 | 2.60 | 6.06 |
| 641 | RMRP | 1.21 | −1.59 | 0.33 | 372 | CYP7A1 | 0.34 | −1.60 | 0.33 |
| 152 | CIDEC | 0.50 | −1.22 | 0.43 | 372 | ENO3 | 0.91 | −1.09 | 0.47 |
| 152 | HMOX1 | 6.16 | 1.66 | 3.16 | 372 | GLA | 1.00 | 1.15 | 2.22 |
| 152 | LDHA | 0.82 | −1.49 | 0.36 | 372 | MOGAT2 | 0.28 | −1.14 | 0.45 |
| 152 | AHSG | 1.28 | −1.08 | 0.47 | 372 | POR | 1.52 | 1.44 | 2.71 |
| 152 | ANGPTL2 | 1.16 | −1.36 | 0.39 | 372 | VLDLR | 2.40 | 1.23 | 2.35 |
| 861 | AMACR | 0.78 | −1.40 | 0.38 | 372 | AHSG | 1.18 | −1.23 | 0.43 |
| 861 | ARG1 | 2.88 | 1.48 | 2.79 | 662 | RMRP | 1.18 | −1.04 | 0.49 |
| 861 | CA2 | 0.93 | −1.05 | 0.48 | 127 | RMRP | 0.88 | 2.44 | 5.43 |
| 861 | CIDEC | 2.15 | 1.02 | 2.03 | 209 | SLC6A6 | 2.02 | 1.45 | 2.73 |
| 861 | CSP1 | 1.52 | 2.14 | 4.41 | 713 | RMRP | 1.28 | 1.57 | 2.97 |
| 861 | CYP21A1 | 0.93 | −1.35 | 0.39 | 308 | ALAS1 | 2.30 | 1.27 | 2.41 |
| 861 | CYP3A4 | 2.46 | 1.98 | 3.94 | 308 | CYP2C19 | 1.17 | 1.00 | 2.00 |
| 861 | ENO3 | 0.98 | −1.24 | 0.42 | 308 | CYP3A4 | 2.26 | 2.65 | 6.28 |
| 861 | G6PC | 1.69 | 1.02 | 2.03 | 308 | CYP7A1 | 0.42 | −1.21 | 0.43 |
| 861 | GYS2 | 1.64 | 2.21 | 4.63 | 308 | GHR | 0.70 | −1.07 | 0.48 |
| 861 | HGD | 1.41 | 1.23 | 2.35 | 308 | POR | 3.01 | 1.44 | 2.71 |
| 861 | HOGA1 | 2.77 | 1.00 | 2.00 | 308 | SLC2A2 | 0.58 | −1.02 | 0.49 |

| | | | | |
|---|---|---|---|---|
| 308 | VLDLR | 2.48 | 1.47 | 2.77 |

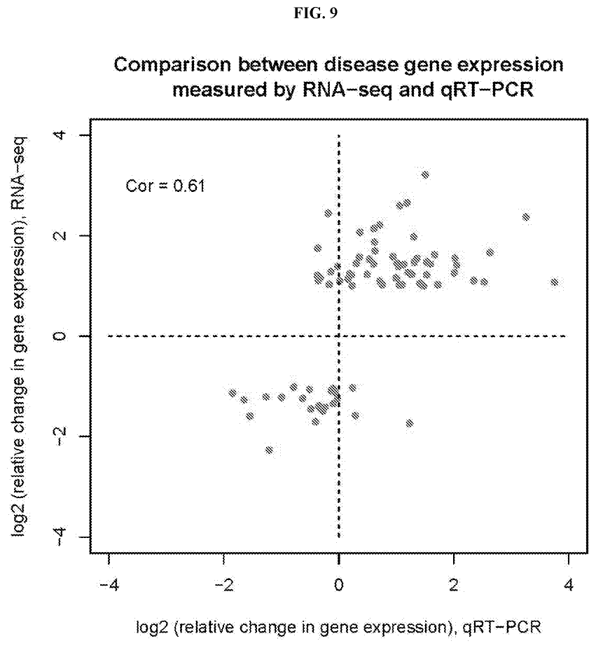

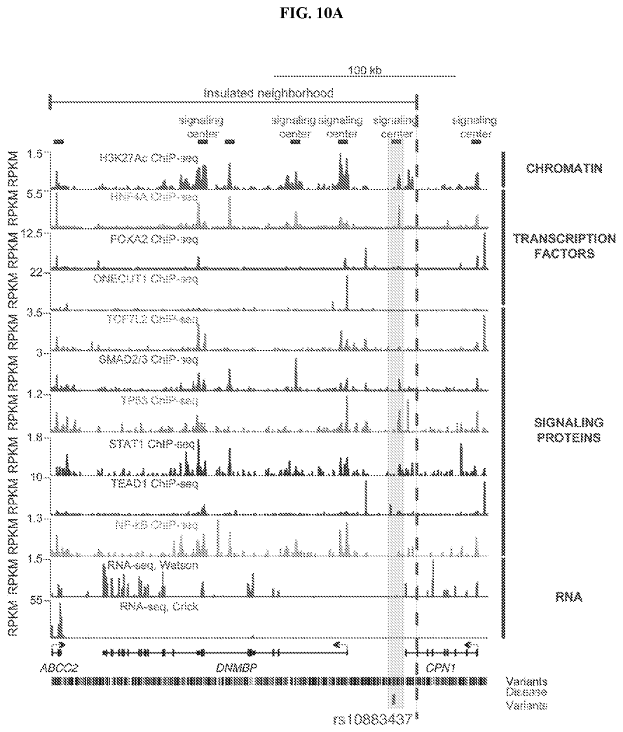

FIG. 9 shows a comparison between DAG expression measured by RNA-seq and qRT-PCR. The scatterplot showed a correlation of 0.61 among the values.

### Example 4. Perturbing Genomic Signaling Centers (GSCs) of Hepatocytes with Failed Phase III Compounds

Forty-five compounds were identified that faded in clinical evaluation due to lack of efficacy as additional perturbation stimuli for hepatocytes. Table 35 shows the targets and associated pathway for each Phase III failure compound.

TABLE 35
Analyzed Phase III failure compounds

| ID | Compound | Target | Pathway |
|---|---|---|---|
| 41 | Enzastaurin | PKC | Epigenetics TGF-beta/Smad |
| 42 | DMXAA | Unclear | Tumor necrosis |
| 43 | BSI-201 | PARP | Cell Cycle/DNA Damage Epigenetics |
| 44 | Darapladib | Phospholipase | Others |
| 45 | Selumetinib | MEK | MAPK/ERK Pathway |
| 46 | Peramivir (trihydrate) | Influenza Virus | Anti-infecticn |
| 47 | Palifosfamide | DNA alkylator/crosslinker | Cell Cycle/DNA Damage |
| 48 | Evacetrapib | CETP | Others |
| 49 | Cediranib | VEGFR | Protein Tyrosine Kinase/RTK |
| 50 | R788 (fostamatinib, disodium | Syk | Protein Tyrosine Kinase/RTK |
| | hexahydrate) | | |
| 51 | Torcetrapib | CETP | Others |
| 52 | Tivozanib | VEGFR | Protein Tyrosine Kinase/RTK |
| 53 | 17-AAG (Tanespimycin) | HSP | Cell Cycle/DNA Damage Metabolic |
| | | | Enzyme/Protease |
| 54 | Zibotentan | Endothelin Receptor | GPCR/G protein |
| 55 | Semagacestat | γ-secretase | Neuronal Signaling Stem Cells/Wnt |
| 56 | Dalcetrapib | CETP | Others |
| 57 | Latrepirdine (dihydrochleride) | AMPK | Epigenetics PI3K/Akt/mTOR |
| 58 | CMX001 (Brincidofovir) | CMV | Anti-infecticn |
| 59 | Vicriviroc (maleate) | CCR | GPCR/G protein Immunology/Inflammation |
| 60 | Temsirolimus | mTOR | PI3K/Akt/mTOR |
| 61 | Preladenant | Adenosine Receptor | GPCR/G protein |
| 62 | EVP-6124 (hydrochloride) | nAChR | Membrane Transporter/Ion Channel |
| | (encenicline) | | |
| 63 | Bitopertin | GlyT1 | Membrane Transporter/Ion Channel |
| 64 | Latrepirdine (dihydrochloride) | AMPK | Epigenetics PI3K/Akt/mTOR |
| 65 | Vanoxerine (dihydrochloride) | Dopamine Reuptake Inhibitor | Neuronal Signaling |
| 66 | CO-1686 (Rociletinib) | EGFR | JAK/STAT Signaling Protein |
| | | | Tyrosine Kinase/RTK |
| 67 | Laropiprant (tredaptive) | Prostaglandin Receptor | GPCR/G protein |
| 68 | Bardoxolone | Keap1-Nrf2 | NF-κB |
| 69 | VX-661 (tezacaptor) | CFTR | Membrane Transporter/Ion Channel |
| 70 | INNO-206 (aldoxorubicin) | Topoisomerase | Cell Cycle/DNA Damage |
| 71 | LY404039 (pomaglumetad | mGluR | GPCR/G protein |
| | methionil (mGlu2/3)) | | |
| 72 | Perifosine (KRX-0401) | AKT | Akt |
| 73 | Cabozantinib (XL184, BMS-907351) | VEGFR2, MET, Ret, Kit, | MET |
| | | Flt-1/3/4, Tie2, and AXL | |
| 74 | Dacomitinib (PF299804, PF299) | EGFR, ErbB2, ErbB4 | AKT/ERK, HER |
| 75 | Pacritinib (SB1518) | FLT3, JAK2, TYK2, JAK3, JAK1 | JAK-STAT |
| 76 | TH-302 (Evofosfamide) | hypoxic regions | Unclear |
| 77 | α-PHP | Unclear | Unclear |
| 78 | LY 2140023 (Pomaglumetad | mGlu₂ & mGlu₃ | Gαi/o protein-dependent |
| | methionil-LY404039) | | |
| 79 | TP-434 (Eravacycline) | Antibiotic resistance mechanisms | Tetracycline-specific efflux |
| 80 | TC-5214 (S-(+)-MecaMylaMine | Nicotinic acetylcholine receptors | Base excision repair and |
| | Hydrochloride) | | homologous recombination repair |
| 81 | Rolofylline (KW-3902) | A1 adenosine receptor | Unclear |
| 82 | Amigal (Deoxygalactonojirimycin | a-galactosidase | Stress signaling |
| | hydrochloride) | | |
| 83 | NOV-002 (oxidized L-Glutathione) | gamma-glutamyl-transpeptidase (GGT) | Glutathione pathway |
| 84 | bms-986094 (inx-189) | NS5B | Unclear |
| 85 | TC-5214 (R-Mecamylamine | Nicotinic receptors | Base excision repair and |
| | hydrochloride) | | homologous recombination repair |

A. RNA-Seq Results

RNA-seq was performed to determine the effects of the compounds on hepatocytes. RNA-seq results were observed to have a 0.99 Pearson correlation for the replicates for each compound having at least 20 million reads each. The mapping ratio was greater than 80% for these reads. Table 36 contains the RNA-seq results for these compounds.

TABLE 36
RNA-seq results for Phase III failure compounds

| Gene | 41 | 43 | 44 | 45 | 46 | 47 | 49 | 50 | 51 | 52 | 53 |
|---|---|---|---|---|---|---|---|---|---|---|---|
| A1BG | 0.13 | −0.21 | 0.01 | 0.00 | 0.02 | −0.13 | 0.15 | 0.01 | −0.38 | 0.51 | −0.10 |
| AARS2 | 0.03 | 0.04 | −0.22 | 0.03 | 0.27 | 0.26 | −0.35 | 0.16 | 0.09 | −0.79 | 0.38 |
| AASS | −0.71 | 0.00 | −0.16 | −0.20 | −0.12 | −0.02 | −0.64 | 0.56 | 0.00 | −1.09 | 0.67 |
| ABAT | −0.94 | −0.22 | −0.63 | −0.26 | −0.09 | −0.13 | −1.60 | −0.28 | 0.30 | −1.69 | −0.05 |
| ABCA1 | −0.31 | −0.05 | −0.69 | 0.21 | −0.04 | 0.07 | −1.68 | 0.15 | 0.73 | −1.14 | 0.71 |
| ABCB11 | −1.34 | 0.49 | −0.47 | 0.41 | 0.20 | 0.04 | −1.44 | 0.21 | 0.51 | −1.28 | 3.63 |
| ABCB4 | −0.92 | −0.15 | −0.13 | −0.02 | 0.01 | −0.22 | −1.21 | 0.29 | 0.38 | −0.14 | 0.90 |
| ABCC2 | −0.06 | 0.34 | −0.08 | 0.08 | 0.02 | −0.02 | −0.57 | 0.31 | 0.60 | −1.42 | 1.54 |
| ABCC3 | −0.02 | 0.30 | −0.11 | 0.29 | 0.32 | 0.18 | −0.68 | −0.32 | 0.23 | −1.31 | −0.57 |
| ABCC6 | −0.86 | 0.07 | −0.13 | 0.69 | 0.47 | 0.18 | −0.59 | 1.23 | 0.36 | −0.60 | 0.52 |
| ABCD1 | −0.68 | −0.12 | −0.45 | 0.15 | 0.25 | 0.26 | −0.75 | −0.10 | 0.12 | −1.52 | −0.16 |
| ABCD3 | −0.96 | −0.21 | −0.73 | −0.26 | −0.28 | −0.23 | −0.83 | 0.17 | 0.01 | −1.27 | 0.74 |
| ABCG5 | −0.76 | 0.14 | −0.07 | 0.04 | 0.09 | 0.03 | −0.49 | 0.59 | −0.02 | −0.77 | −0.46 |
| ABCG8 | −1.08 | −0.02 | −0.49 | −0.09 | 0.06 | 0.02 | −0.93 | 0.96 | −0.02 | −1.36 | 0.68 |
| ABHD5 | 0.25 | −0.15 | 0.39 | 0.13 | −0.22 | −0.12 | 0.97 | −0.35 | 0.09 | 1.29 | 1.17 |
| ACACA | 0.61 | 0.11 | 0.48 | −0.15 | 0.04 | 0.07 | −0.50 | 0.42 | 0.58 | −0.95 | 0.97 |
| ACACB | −0.80 | 0.06 | 0.30 | 0.43 | 0.36 | 0.02 | 0.06 | 1.01 | 0.12 | 0.62 | 1.54 |
| ACAD9 | 0.04 | −0.13 | −0.09 | −0.03 | 0.00 | −0.05 | −0.28 | 0.24 | 0.05 | −1.12 | −0.22 |
| ACADSB | −0.67 | −0.21 | −0.54 | −0.33 | −0.29 | −0.14 | −0.74 | 0.74 | 0.27 | −0.38 | 1.23 |
| ACADVL | −0.14 | 0.22 | 0.10 | 0.38 | 0.30 | 0.19 | −0.03 | 0.13 | −0.44 | −0.87 | −1.05 |
| ACOT8 | −0.46 | −0.21 | −0.10 | −0.20 | −0.13 | −0.19 | 0.11 | −0.09 | −0.50 | −0.03 | −0.43 |
| ACOX2 | −1.42 | −0.30 | −0.48 | −0.46 | −0.03 | −0.29 | −1.23 | 0.52 | −0.29 | −0.35 | 0.95 |
| ACOX3 | −0.13 | −0.10 | −0.60 | 0.31 | −0.01 | −0.04 | −0.22 | −0.58 | 0.14 | −0.39 | −0.81 |
| ACSF3 | 0.02 | −0.04 | 0.00 | −0.04 | 0.08 | 0.01 | −0.06 | 0.13 | −0.22 | −0.16 | −0.12 |
| ACSL1 | 0.30 | −0.10 | 0.45 | 0.24 | −0.03 | 0.00 | −0.03 | 0.76 | 0.33 | −0.03 | 1.10 |
| ACSL3 | −0.04 | −0.13 | 0.24 | −0.12 | −0.34 | −0.18 | −0.12 | 0.23 | 0.16 | 0.01 | 1.02 |
| ACTG2 | −1.22 | −1.12 | −1.36 | −1.48 | −1.66 | −1.64 | 1.03 | 1.20 | 2.16 | 0.67 | 1.83 |
| ACTN1 | −0.08 | 0.03 | −0.16 | 0.23 | −0.03 | 0.01 | −0.27 | −0.57 | 0.51 | −0.86 | −0.55 |
| ADA | −0.56 | −0.17 | −0.07 | −0.18 | 0.16 | −0.02 | −0.38 | −0.70 | −0.03 | −0.30 | −0.13 |
| ADCK3 | −1.19 | −0.07 | −0.42 | −0.25 | −0.02 | −0.03 | −0.37 | 0.71 | 0.00 | −0.48 | 1.04 |
| ADK | −0.07 | −0.33 | −0.44 | −0.42 | −0.46 | −0.35 | −0.32 | −0.28 | −0.38 | −0.22 | 0.06 |
| ADSL | −0.34 | −0.28 | −0.49 | −0.46 | −0.36 | −0.25 | −0.27 | −0.39 | −0.24 | −0.67 | −0.56 |
| AFP | −0.72 | −0.26 | −1.26 | −0.08 | −0.31 | −0.29 | 0.11 | −1.06 | −0.95 | −1.03 | 0.26 |
| AGK | 0.05 | −0.11 | −0.45 | −0.22 | −0.24 | −0.21 | −0.54 | −0.14 | 0.04 | −0.88 | 0.15 |
| AGL | −0.25 | −0.04 | −0.28 | −0.11 | −0.20 | −0.04 | −0.42 | 0.57 | 0.21 | −0.58 | 0.49 |
| AGPAT2 | −0.33 | −0.08 | 0.33 | 0.52 | 0.36 | 0.33 | 0.50 | −0.55 | −0.55 | 0.27 | −0.98 |
| AGPS | −0.81 | −0.21 | −0.38 | −0.33 | −0.31 | −0.19 | −0.86 | −0.19 | 0.32 | −1.03 | 0.20 |
| AGT | −0.05 | −0.24 | −0.20 | 0.03 | −0.08 | −0.13 | −0.34 | 0.27 | −0.30 | 0.03 | 0.08 |
| AGTR1 | −0.76 | −0.22 | −0.18 | −0.55 | −0.40 | −0.67 | −0.32 | 0.25 | −0.45 | −0.47 | −0.03 |
| AGXT | −0.27 | −0.06 | −0.38 | 0.22 | 0.44 | 0.30 | −0.23 | 0.45 | −0.25 | 0.04 | 0.27 |
| AHI1 | 0.25 | 0.33 | 0.12 | 0.01 | −0.18 | 0.06 | −0.17 | 0.39 | 0.19 | 0.24 | 0.39 |
| AHSG | −0.31 | −0.31 | −0.23 | −0.20 | 0.03 | −0.17 | −0.40 | 0.14 | −0.25 | 0.09 | 0.07 |
| AK1 | −0.71 | −0.22 | −0.25 | 0.01 | −0.06 | −0.02 | 0.26 | −0.42 | −0.58 | −0.10 | −1.12 |
| AKR1C1 | −1.80 | −0.12 | −0.30 | −0.09 | −0.24 | −0.16 | −0.29 | −0.46 | −0.53 | −0.90 | 0.55 |
| AKR1C2 | −1.87 | −0.05 | −0.22 | 0.17 | 0.07 | 0.05 | −0.53 | −0.30 | −0.34 | −1.14 | 0.87 |
| AKR1C4 | −2.28 | −0.34 | −1.37 | −0.30 | −0.41 | −0.56 | −0.89 | 0.84 | −0.68 | −1.22 | 0.94 |
| AKR1D1 | 1.94 | −0.11 | 1.24 | 0.62 | −0.04 | −0.12 | 0.36 | 1.67 | 0.14 | 0.98 | 2.91 |
| ALAD | −1.41 | −0.24 | −0.73 | 0.24 | 0.07 | −0.09 | −0.68 | 1.41 | 0.17 | −0.88 | 2.04 |
| ALAS1 | −0.37 | −0.03 | −0.27 | −0.07 | −0.13 | −0.04 | −0.19 | 0.60 | 0.30 | −0.82 | 2.49 |
| ALDH1B1 | −0.26 | −0.12 | −0.27 | 0.17 | 0.10 | 0.06 | 0.42 | 0.38 | 0.32 | −0.05 | 0.18 |
| ALDH2 | −0.39 | −0.20 | −0.21 | 0.10 | 0.07 | −0.01 | −0.11 | 0.26 | −0.28 | 0.18 | 0.22 |
| ALDH6A1 | −0.94 | −0.44 | −0.43 | −0.51 | −0.48 | −0.48 | −0.50 | 0.56 | −0.02 | 0.14 | 0.80 |
| ALDOA | 0.06 | 0.01 | 0.02 | 0.02 | 0.01 | 0.09 | −0.02 | −0.05 | −0.41 | −1.36 | −0.23 |
| ALDOB | 0.22 | −0.24 | −0.14 | 0.29 | −0.08 | −0.20 | −0.15 | 3.35 | −0.01 | 0.36 | 1.99 |
| ALG12 | −0.21 | −0.07 | −0.03 | −0.03 | 0.02 | 0.00 | −0.48 | 0.38 | −0.06 | −1.02 | 0.22 |
| ALMS1 | −0.07 | 0.03 | 0.18 | 0.02 | −0.05 | −0.11 | −0.42 | 0.03 | 0.80 | −0.65 | 0.99 |
| ALPL | −1.25 | 0.11 | 0.28 | 0.40 | −0.08 | −0.07 | −0.12 | 0.83 | −0.14 | −0.04 | 1.83 |
| AMACR | 0.90 | −0.32 | 0.06 | 0.18 | −0.17 | −0.11 | 0.84 | 0.47 | −0.05 | 0.00 | 0.36 |
| AMPD3 | −0.35 | 0.19 | 0.42 | 0.22 | 0.13 | −0.01 | −0.52 | −0.90 | 0.28 | −0.18 | 1.15 |
| AMT | −0.42 | −0.04 | −0.35 | 0.29 | 0.24 | 0.13 | −0.23 | −0.28 | −0.11 | 0.50 | 0.31 |
| ANGPTL3 | −2.31 | −0.14 | −1.20 | −0.67 | −0.42 | −0.28 | −1.40 | 0.24 | −0.46 | −1.56 | 0.06 |
| ANKS4B | −0.71 | −0.24 | −0.52 | −0.06 | −0.06 | −0.16 | −0.31 | 0.49 | −0.02 | −0.68 | 1.12 |
| ANKS6 | 0.10 | 0.04 | 0.11 | −0.02 | 0.15 | 0.12 | 0.09 | 0.03 | 0.19 | −0.21 | −0.15 |
| ANPEP | −0.52 | −0.09 | 0.11 | 0.04 | 0.07 | −0.12 | −0.85 | 0.49 | −0.19 | −1.04 | −0.06 |
| AOC2 | 0.11 | 0.36 | 0.28 | 0.28 | 0.42 | 0.34 | −0.47 | 0.64 | 0.53 | −0.20 | 0.24 |
| AP3B1 | −0.31 | −0.21 | −0.13 | −0.21 | −0.40 | −0.31 | −0.75 | −0.01 | 0.04 | −1.15 | 0.27 |
| APC | −0.11 | 0.04 | 0.11 | −0.04 | −0.20 | −0.05 | −0.11 | 0.35 | 0.47 | −0.32 | 0.58 |
| APOA1 | −0.02 | −0.26 | −0.16 | −0.05 | −0.02 | −0.13 | 0.41 | −0.18 | −0.67 | 0.73 | −0.47 |
| APOA2 | −0.48 | −0.52 | −0.33 | −0.45 | −0.52 | −0.53 | 0.16 | −0.31 | −0.76 | 0.31 | −0.64 |
| APOA5 | −0.04 | −0.17 | −0.19 | 0.13 | 0.13 | 0.00 | −0.08 | 0.97 | −0.36 | −0.09 | 0.07 |
| APOB | −0.19 | 0.03 | 0.54 | −0.10 | −0.10 | −0.23 | −0.99 | 1.87 | 0.90 | −0.40 | 2.13 |
| APOC2 | −0.56 | −0.28 | −0.28 | −0.12 | −0.17 | −0.25 | −0.17 | 0.14 | −0.85 | 0.11 | −0.59 |
| APOC3 | −0.15 | −0.42 | −0.41 | −0.10 | −0.06 | −0.18 | 0.16 | 0.49 | −0.56 | 0.50 | −0.21 |
| APOE | −0.02 | −0.24 | 0.00 | 0.04 | 0.04 | −0.13 | 0.42 | 0.11 | −0.78 | 0.68 | −0.32 |
| APRT | 0.06 | −0.04 | −0.23 | 0.06 | 0.23 | 0.26 | 0.19 | −0.41 | −0.55 | 0.00 | −1.15 |
| AQP7 | −1.99 | −0.17 | 0.16 | 0.14 | −0.41 | −0.35 | 0.82 | 1.66 | 0.64 | 0.50 | 1.89 |
| AQP9 | 0.08 | −0.22 | 0.41 | −0.06 | −0.29 | −0.25 | 0.06 | 0.21 | 0.23 | 0.56 | 0.36 |
| ARG1 | −0.26 | −0.20 | −0.64 | 0.17 | 0.02 | −0.13 | −0.34 | 3.76 | −0.20 | 0.74 | 1.93 |
| ARG2 | −0.79 | −0.10 | 0.33 | −0.64 | −0.34 | −0.35 | −0.69 | −0.23 | −0.03 | 0.47 | 0.98 |
| ARL13B | −0.40 | −0.06 | −0.10 | −0.19 | −0.24 | −0.10 | −0.21 | −0.40 | 0.01 | 0.16 | −0.51 |
| ARSA | 0.15 | 0.30 | 0.33 | 0.44 | 0.49 | 0.41 | 0.11 | 0.02 | −0.18 | −0.57 | −1.20 |
| ARSB | −0.15 | −0.11 | 0.09 | −0.35 | 0.03 | 0.05 | −0.93 | −0.13 | 0.16 | −1.00 | 0.05 |
| ASL | −0.20 | 0.01 | 0.27 | 0.01 | 0.25 | 0.17 | 0.03 | −0.02 | −0.27 | 0.18 | −0.43 |
| ASNS | 0.71 | −0.26 | 0.50 | 0.11 | −0.22 | −0.11 | 0.59 | −0.58 | 0.01 | 1.24 | 1.26 |
| ASPA | −1.73 | −0.02 | −0.59 | 0.56 | −0.10 | −0.25 | 0.67 | 1.23 | 0.05 | 0.69 | 0.76 |
| ASXL1 | −0.41 | 0.15 | −0.08 | 0.08 | 0.09 | 0.11 | −0.33 | −0.08 | 0.08 | −0.79 | 0.61 |
| ATG7 | −0.05 | −0.04 | −0.11 | −0.03 | −0.05 | 0.03 | −0.27 | −0.11 | −0.05 | −0.54 | −0.10 |
| ATIC | −0.17 | −0.15 | −0.48 | −0.39 | −0.26 | −0.18 | −0.46 | −0.25 | −0.19 | −0.83 | −0.42 |
| ATM | 0.35 | 0.17 | 0.12 | 0.13 | 0.16 | 0.15 | −0.30 | 0.52 | 0.66 | −0.13 | 0.54 |
| ATP7A | 0.13 | 0.02 | −0.02 | 0.05 | −0.27 | 0.00 | −0.20 | 0.10 | 0.35 | −0.61 | 0.45 |
| ATP7B | −0.16 | −0.04 | −0.19 | 0.01 | 0.00 | −0.02 | −0.83 | 0.66 | 0.43 | −1.57 | 0.52 |
| ATP8B1 | −0.33 | −0.13 | −0.03 | −0.16 | −0.26 | −0.18 | −0.32 | −0.63 | 0.51 | −0.59 | 0.28 |
| B2M | −0.26 | −0.51 | −0.16 | −0.36 | −0.52 | −0.48 | 0.04 | −0.13 | −0.32 | 0.13 | −0.56 |
| B9D2 | −0.61 | 0.25 | −0.32 | −0.16 | 0.35 | 0.39 | 0.51 | 0.04 | −0.17 | −0.21 | 0.27 |
| BAAT | 0.38 | −0.20 | 0.22 | 0.02 | −0.23 | −0.12 | 0.29 | 1.23 | 0.03 | −0.63 | 1.09 |
| BBOX1 | 0.24 | −0.42 | −0.66 | −0.15 | −0.71 | −0.57 | −0.62 | −0.52 | −0.82 | −0.31 | 1.23 |
| BBS10 | −0.41 | −0.24 | −0.41 | −0.24 | −0.36 | −0.16 | −0.88 | 0.09 | 0.27 | −0.81 | 0.28 |
| BCHE | −0.54 | −0.37 | −0.64 | −0.38 | −0.32 | −0.38 | −1.14 | 0.37 | −0.14 | −0.60 | 0.82 |
| BCKDHB | −0.29 | −0.11 | −0.57 | −0.13 | −0.10 | −0.03 | −0.47 | 0.43 | −0.12 | −0.42 | 0.08 |
| BCKDK | −0.10 | 0.01 | −0.13 | −0.20 | −0.14 | −0.05 | −0.35 | 0.20 | −0.17 | −1.15 | −0.42 |
| BCO1 | 0.19 | −0.20 | −0.24 | −0.14 | −0.07 | −0.05 | −0.71 | 0.33 | −0.43 | −1.83 | 0.69 |
| BCR | −0.26 | −0.10 | 0.03 | −0.13 | −0.02 | −0.02 | −0.45 | −0.25 | −0.01 | −0.96 | −0.12 |
| BLNK | 0.15 | −0.01 | 0.58 | 0.01 | −0.02 | −0.05 | 0.51 | 0.94 | 0.07 | 0.96 | 0.72 |
| BLOC1S3 | 0.20 | −0.12 | 0.30 | 0.17 | 0.01 | 0.11 | 0.00 | 0.45 | 0.09 | 0.04 | 0.27 |
| BMPR1A | −0.20 | −0.20 | −0.22 | −0.01 | −0.14 | −0.25 | −0.29 | 0.17 | 0.52 | −0.61 | 0.61 |
| BRAF | 0.09 | 0.00 | 0.05 | 0.07 | −0.13 | −0.25 | −0.36 | 0.19 | 0.39 | −0.45 | 1.19 |
| BSCL2 | −1.67 | −1.20 | −1.17 | −1.11 | −1.57 | −1.59 | 0.41 | 0.14 | −2.88 | 0.60 | −3.35 |
| BTD | −1.04 | −0.20 | −0.47 | −0.28 | −0.14 | −0.29 | −0.61 | 0.50 | 0.08 | −0.51 | 1.00 |
| C10orf10 | −1.11 | −0.30 | 0.08 | −0.01 | 0.08 | 0.00 | −0.19 | 0.17 | −0.33 | −0.70 | −0.55 |
| C10orf11 | 0.13 | −0.13 | 0.04 | 0.10 | −0.18 | −0.37 | 0.27 | −0.54 | −0.22 | 0.64 | −0.19 |
| C15orf41 | −0.17 | −0.39 | −0.35 | −0.38 | −0.25 | −0.26 | −0.16 | −0.11 | 0.16 | −0.44 | 0.62 |
| C4A | 0.43 | 0.22 | 0.27 | 0.72 | 0.79 | 0.40 | −1.01 | 0.58 | 0.68 | −1.15 | 0.09 |
| C9 | −0.58 | −0.42 | −0.46 | −0.44 | −0.57 | −0.55 | −0.71 | 0.24 | −0.01 | −0.63 | 0.34 |
| CA12 | −0.32 | −0.03 | 0.76 | 0.05 | −0.29 | −0.24 | 0.50 | −0.03 | 0.29 | 0.01 | 2.47 |
| CA2 | 1.28 | −0.29 | 0.46 | 0.04 | −0.12 | −0.18 | 1.36 | 1.51 | 0.28 | 0.82 | 1.63 |
| CA5A | −0.19 | −0.04 | −0.20 | 0.07 | 0.03 | 0.03 | −0.04 | 1.68 | −0.10 | −0.37 | 1.86 |
| CAV1 | −1.59 | −0.29 | −0.91 | −0.18 | −0.36 | −0.26 | −0.40 | −0.62 | 0.19 | −0.36 | −0.27 |
| CCND1 | 0.55 | 0.07 | −0.46 | −0.89 | −0.01 | 0.07 | −2.40 | −1.92 | 0.40 | −1.69 | −2.47 |
| CD320 | −0.15 | −0.14 | −0.13 | −0.27 | −0.01 | 0.09 | −0.15 | −0.66 | −0.62 | −0.41 | −1.02 |
| CD46 | −0.11 | −0.16 | 0.09 | −0.36 | −0.29 | −0.22 | −0.21 | 0.26 | 0.16 | 0.28 | 0.45 |
| CD81 | −0.10 | −0.24 | −0.16 | −0.16 | −0.08 | −0.17 | −0.06 | −0.24 | −0.41 | −0.05 | −0.42 |
| CDAN1 | −0.06 | 0.03 | 0.06 | 0.05 | 0.23 | 0.22 | −0.37 | 0.04 | 0.43 | −0.88 | 0.36 |
| CDH1 | −1.52 | −0.15 | −0.34 | −0.13 | 0.05 | −0.23 | −0.87 | 0.60 | 0.34 | −0.22 | 0.67 |
| CEP164 | 0.27 | 0.30 | 0.13 | 0.31 | 0.39 | 0.32 | −0.09 | −0.10 | 0.03 | −0.74 | −0.77 |
| CEP19 | −0.44 | −0.08 | −0.17 | −0.38 | −0.09 | −0.18 | −0.36 | −0.30 | −0.31 | −0.17 | −0.96 |
| CEP290 | 0.50 | 0.08 | −0.15 | −0.18 | −0.30 | −0.14 | −0.02 | −0.36 | −0.07 | 0.31 | 0.11 |
| CEP83 | 0.74 | −0.05 | −0.18 | −0.23 | −0.20 | −0.26 | −0.10 | 0.29 | 0.20 | −0.27 | −0.07 |
| CFB | −0.12 | −0.22 | 0.09 | −0.01 | −0.13 | −0.26 | −0.47 | 0.30 | −0.08 | −0.15 | 0.05 |
| CFHR2 | −0.65 | −0.53 | −0.70 | −0.30 | −0.38 | −0.35 | −0.41 | 0.35 | −0.57 | 0.17 | 0.14 |
| CHRM3 | 0.93 | 0.16 | 0.31 | −0.37 | −0.12 | −0.22 | −0.35 | 0.13 | −0.27 | −0.94 | 0.76 |
| CIDEC | −1.60 | −0.27 | −0.87 | 0.45 | −0.45 | 0.05 | −0.44 | −0.66 | −0.44 | −1.36 | 0.12 |
| CLDN1 | −1.33 | −0.17 | −0.72 | −0.62 | −0.36 | −0.33 | −1.35 | −0.58 | −0.06 | −1.39 | −1.24 |
| COG1 | −0.20 | 0.03 | −0.15 | −0.14 | −0.10 | −0.08 | −0.60 | 0.08 | 0.06 | −1.16 | 0.26 |
| COG5 | −0.13 | −0.02 | −0.19 | −0.25 | −0.36 | −0.14 | −0.41 | −0.05 | 0.19 | −0.38 | 0.44 |
| COG6 | −0.43 | −0.22 | −0.13 | −0.42 | −0.54 | −0.34 | −0.32 | 0.32 | 0.25 | 0.09 | 1.17 |
| COL1A1 | −0.30 | 0.04 | 0.63 | 1.13 | 0.33 | 0.17 | −0.59 | −0.31 | 0.07 | −1.24 | 0.13 |
| COX6A1 | −0.13 | −0.31 | −0.13 | 0.18 | 0.39 | 0.18 | 0.12 | −0.26 | −0.83 | 0.24 | −0.78 |
| CP | −0.08 | 0.00 | 0.92 | −0.06 | −0.09 | −0.32 | −0.76 | 0.74 | 0.46 | −0.02 | 1.25 |
| CPS1 | 0.00 | −0.26 | −0.80 | 0.21 | 0.01 | −0.23 | −0.88 | 5.91 | 0.23 | −0.46 | 3.68 |
| CPT1A | −1.00 | 0.15 | 0.21 | 0.12 | −0.10 | −0.21 | 0.08 | 0.15 | −0.41 | −0.82 | −1.07 |
| CPT2 | −0.31 | −0.13 | −0.30 | −0.10 | −0.19 | −0.14 | −0.16 | 0.47 | −0.28 | −0.46 | −0.31 |
| CROT | −1.01 | −0.23 | −0.73 | −0.09 | −0.25 | −0.11 | −0.63 | −0.09 | −0.01 | −0.70 | 0.14 |
| CSPP1 | 0.38 | 0.21 | 0.30 | 0.04 | −0.06 | 0.03 | −0.13 | −0.09 | −0.17 | −0.16 | 0.08 |
| CTC1 | 0.47 | 0.08 | 0.14 | 0.01 | 0.10 | 0.08 | −0.12 | 0.51 | 0.53 | −0.59 | −0.17 |
| CTDSP2 | −1.09 | −0.21 | 0.11 | 0.08 | 0.00 | −0.07 | −0.45 | 0.46 | 0.20 | −0.52 | 0.81 |
| CTH | 0.54 | −0.07 | 0.71 | 0.24 | 0.06 | 0.03 | 0.61 | 0.64 | −0.16 | 1.68 | 2.18 |
| CTNS | 0.65 | 0.15 | 0.59 | −0.17 | −0.25 | −0.14 | 0.52 | 0.21 | 0.14 | 0.03 | −0.32 |
| CTSF | −0.60 | −0.08 | 0.26 | 0.15 | 0.21 | 0.09 | 0.15 | 0.05 | −0.28 | 0.50 | 0.31 |
| CTSK | −0.08 | 0.09 | 0.31 | 0.14 | 0.28 | 0.10 | 0.25 | 0.66 | 0.38 | −0.60 | −0.60 |
| CYB5A | −0.08 | −0.14 | −0.35 | 0.08 | 0.01 | −0.04 | 0.07 | 0.95 | 0.13 | 0.37 | 0.84 |
| CYB5R3 | −0.31 | −0.15 | 0.08 | 0.07 | 0.00 | 0.05 | −0.38 | 0.23 | 0.00 | −1.02 | −0.08 |
| CYP27A1 | −0.56 | −0.28 | −0.17 | −0.03 | −0.21 | −0.30 | −0.23 | 0.71 | −0.02 | 0.13 | 0.29 |
| CYP2B6 | 0.42 | 0.07 | −0.12 | 0.38 | 0.07 | 0.03 | −0.46 | 0.55 | 0.36 | 0.16 | 1.32 |
| CYP2C19 | 0.47 | 0.30 | −0.01 | 0.76 | 0.25 | 0.12 | −0.80 | 0.61 | 0.61 | −1.36 | 0.34 |
| CYP2D6 | −0.28 | −0.15 | −0.21 | 0.03 | 0.24 | 0.10 | −0.12 | 0.31 | −0.16 | 0.21 | 0.06 |
| CYP39A1 | −1.39 | −0.20 | −0.23 | −0.27 | −0.20 | −0.43 | −0.61 | 1.11 | 0.03 | 0.94 | 1.38 |
| CYP3A4 | 0.22 | −0.46 | −0.47 | 0.08 | −0.53 | −0.49 | −0.75 | 3.59 | 1.87 | 0.50 | 2.50 |
| CYP7B1 | 0.06 | 0.03 | −0.03 | −0.15 | −0.16 | −0.21 | −0.93 | 0.59 | 0.26 | −1.02 | 0.43 |
| DAO | −0.27 | −0.34 | 0.04 | −0.34 | 0.00 | −0.04 | −0.61 | 0.69 | −0.33 | −0.04 | 0.33 |
| DBT | −0.39 | −0.21 | −0.22 | −0.18 | −0.24 | −0.13 | −0.39 | 0.41 | 0.34 | 0.00 | 1.12 |
| DCDC2 | −0.58 | −0.59 | −1.41 | −0.56 | −0.04 | −0.18 | −1.44 | −0.45 | 0.27 | −2.42 | −0.55 |
| DCXR | −0.48 | −0.32 | −0.59 | −0.29 | −0.20 | −0.24 | 0.06 | 0.06 | −0.63 | 0.36 | −0.31 |
| DDC | −0.14 | −0.22 | −0.97 | −0.28 | −0.41 | −0.24 | −1.20 | 0.24 | −0.52 | −0.99 | 0.41 |
| DDO | 0.14 | −0.22 | −0.36 | −0.08 | 0.12 | −0.04 | −0.55 | 0.41 | −0.12 | −1.46 | 0.40 |
| DENR | −0.25 | −0.18 | −0.37 | −0.37 | −0.36 | −0.18 | −0.16 | −0.59 | −0.24 | −0.33 | −0.35 |
| DHCR24 | −0.48 | −0.14 | 0.32 | −0.42 | −0.16 | −0.20 | −0.51 | −0.05 | 0.24 | −0.94 | 1.34 |
| DHCR7 | 0.60 | 0.01 | 1.00 | −0.02 | 0.04 | 0.05 | 0.49 | 0.07 | 0.15 | 0.23 | 1.21 |
| DHODH | 0.36 | 0.01 | −0.35 | −0.03 | 0.27 | 0.20 | −0.38 | −0.04 | −0.20 | −0.52 | −0.39 |
| DIS3L2 | −0.05 | −0.13 | −0.37 | −0.22 | −0.14 | −0.14 | −0.59 | 0.10 | 0.27 | −1.20 | 0.49 |
| DKC1 | 0.17 | −0.04 | −0.40 | −0.29 | −0.11 | −0.05 | −0.27 | −0.45 | −0.21 | −0.39 | −0.57 |
| DLAT | 0.17 | −0.09 | −0.10 | −0.02 | −0.15 | −0.01 | 0.15 | −0.13 | −0.01 | −0.64 | −0.08 |
| DLD | −0.67 | −0.23 | −0.35 | −0.25 | −0.35 | −0.20 | −0.48 | 0.04 | −0.01 | −1.04 | 0.00 |
| DMGDH | −0.58 | −0.15 | −0.59 | −0.13 | −0.11 | −0.16 | −0.80 | 0.68 | 0.20 | −0.39 | 1.01 |
| DNAH5 | −0.76 | 0.11 | −0.06 | −0.11 | −0.05 | −0.12 | −0.46 | −0.59 | 0.53 | −0.02 | −0.41 |
| DNM1L | −0.54 | −0.15 | −0.26 | −0.20 | −0.28 | −0.18 | −0.37 | 0.00 | 0.19 | −1.03 | 0.24 |
| DOLK | −0.05 | −0.03 | 0.04 | −0.05 | 0.09 | 0.06 | −0.22 | 0.06 | −0.09 | −0.83 | −0.66 |
| DPM3 | −0.02 | −0.33 | 0.02 | −0.05 | −0.11 | −0.18 | 0.91 | −0.35 | −0.65 | 1.21 | −0.43 |
| DPYD | −0.66 | −0.17 | −0.29 | −0.08 | −0.21 | −0.22 | −0.89 | 0.63 | 0.43 | −0.45 | 1.00 |
| DPYS | −1.05 | −0.12 | −0.65 | −0.10 | −0.01 | −0.18 | −1.15 | 0.87 | −0.15 | −0.68 | 0.89 |
| EARS2 | 0.28 | 0.12 | −0.24 | 0.04 | 0.04 | 0.15 | −0.56 | −0.01 | 0.34 | −0.90 | 1.07 |
| EDA | 0.59 | 0.00 | 0.72 | −0.28 | −0.03 | 0.01 | 0.24 | 0.37 | 0.72 | 0.92 | 1.35 |
| EHHADH | −0.36 | −0.21 | −0.50 | −0.02 | −0.10 | −0.14 | −0.60 | 1.17 | 0.11 | −0.55 | 0.67 |
| ELAC2 | −0.29 | −0.08 | −0.29 | −0.25 | −0.15 | −0.09 | −0.36 | 0.06 | −0.18 | −1.27 | −0.29 |
| ENO3 | −0.53 | −0.03 | −0.11 | 0.64 | 0.30 | 0.20 | −0.63 | −0.61 | −0.44 | −0.94 | −1.63 |
| ENPP1 | 0.16 | −0.13 | −0.16 | 0.06 | −0.28 | −0.33 | −0.21 | 0.83 | 0.31 | 0.00 | 1.38 |
| EPG5 | 0.33 | 0.05 | 0.65 | 0.11 | 0.13 | 0.09 | 0.12 | 0.38 | 0.63 | −0.40 | 1.37 |
| EPHX1 | −0.34 | −0.10 | −0.36 | −0.03 | −0.26 | −0.30 | −0.54 | 0.55 | −0.07 | −0.62 | 1.45 |
| ERCC4 | 0.13 | −0.01 | 0.06 | −0.13 | −0.23 | −0.11 | −0.08 | −0.05 | 0.21 | 0.02 | 0.02 |
| ERCC6 | −0.23 | 0.01 | 0.07 | 0.12 | 0.05 | 0.07 | −0.41 | 0.38 | 0.42 | −0.49 | 1.24 |
| ETFB | −0.63 | −0.11 | −0.24 | 0.02 | 0.23 | 0.13 | 0.04 | −0.35 | −0.56 | 0.16 | −0.56 |
| ETHE1 | 0.54 | −0.18 | 0.10 | 0.12 | 0.31 | 0.03 | −0.19 | 0.15 | −0.34 | 0.14 | −0.86 |
| F2 | −0.23 | −0.18 | 0.14 | 0.22 | 0.11 | −0.02 | −0.48 | −0.07 | −0.15 | −0.30 | 0.01 |
| F2RL1 | −0.16 | −0.47 | 0.00 | −0.79 | −0.53 | −0.49 | −0.29 | −0.87 | −0.34 | −0.03 | −0.23 |
| F5 | −0.13 | 0.01 | 0.10 | 0.06 | −0.08 | −0.16 | −1.17 | 0.83 | 0.36 | −0.68 | 1.01 |
| F9 | −0.62 | −0.33 | −0.46 | −0.40 | −0.53 | −0.40 | −0.77 | 0.32 | 0.11 | −0.51 | 0.54 |
| FABP1 | −0.85 | −0.36 | −0.59 | 0.48 | 0.73 | 0.44 | 0.31 | −0.14 | −0.84 | 0.71 | −0.51 |
| FADD | −0.35 | −0.16 | −0.16 | −0.27 | −0.14 | −0.16 | −0.13 | −0.69 | −0.49 | −0.29 | −1.26 |
| FAM111A | 0.44 | 0.05 | 0.02 | 0.05 | −0.08 | −0.03 | 0.17 | 0.54 | 0.47 | −0.20 | 0.55 |
| FAM111B | 0.70 | 0.78 | −0.81 | −1.74 | 0.11 | 1.01 | −2.18 | −1.87 | −0.29 | −2.22 | −2.06 |
| FAN1 | 0.28 | 0.07 | −0.25 | −0.20 | −0.06 | 0.08 | −0.32 | 0.46 | 0.47 | −0.43 | 0.02 |
| FARS2 | −0.30 | −0.13 | −0.16 | −0.28 | −0.22 | −0.16 | −0.29 | −0.29 | −0.30 | −0.41 | −0.44 |
| FASLG | 0.02 | 0.55 | 0.02 | 0.45 | 1.15 | 0.91 | −0.64 | −1.35 | 0.07 | −1.93 | 1.73 |
| FGA | −1.25 | −0.40 | −0.74 | −0.34 | −0.25 | −0.37 | −1.10 | 2.03 | −0.25 | −1.19 | 1.39 |
| FGD1 | −0.89 | −0.16 | −0.39 | −0.18 | −0.25 | −0.14 | −0.21 | −0.60 | 0.10 | −1.11 | 1.43 |
| FGFR2 | −2.50 | −0.50 | −0.53 | −0.45 | −0.13 | −0.50 | −1.14 | 0.76 | 0.14 | 0.08 | 1.21 |
| FIG4 | 0.00 | −0.18 | 0.19 | −0.21 | −0.30 | −0.26 | 0.05 | 0.09 | 0.13 | −0.59 | 0.69 |
| FIS1 | −0.10 | −0.09 | −0.01 | 0.14 | 0.44 | 0.35 | 0.14 | −0.33 | −0.59 | −0.16 | −0.68 |
| FTCD | 0.20 | 0.22 | 0.40 | 0.31 | 0.58 | 0.30 | 0.29 | −0.25 | −0.26 | −0.24 | −0.18 |
| FTH1 | −0.08 | 0.00 | 0.59 | 0.00 | −0.04 | −0.02 | 0.58 | −0.29 | −0.76 | 0.39 | 0.09 |
| FUCA1 | −0.42 | −0.31 | −0.29 | −0.30 | −0.23 | −0.27 | −0.53 | 0.26 | −0.29 | −0.59 | 0.22 |
| FXN | −0.56 | −0.24 | −0.61 | −0.70 | −0.69 | −0.54 | −0.41 | −0.27 | −0.48 | −0.02 | −0.38 |
| G6PC | −1.03 | −0.02 | 0.44 | 0.50 | 0.23 | −0.33 | −0.35 | 4.61 | 0.35 | 0.14 | 3.65 |
| G6PC3 | −0.04 | 0.17 | −0.32 | −0.08 | 0.06 | 0.04 | −0.29 | −0.48 | −0.32 | −0.99 | −1.43 |
| G6PD | 0.63 | 0.31 | 0.29 | 0.07 | 0.08 | −0.04 | −0.38 | 0.01 | −0.26 | −1.72 | −0.71 |
| GALK1 | −0.18 | 0.02 | 0.11 | 0.00 | 0.18 | 0.15 | 0.08 | −0.33 | −0.51 | 0.43 | −1.11 |
| GALK2 | −0.16 | −0.18 | 0.02 | −0.17 | −0.13 | −0.21 | 0.33 | 0.26 | −0.24 | 0.30 | −0.06 |
| GALT | −0.21 | 0.07 | 0.35 | 0.11 | 0.15 | −0.09 | 0.36 | 0.75 | −0.25 | −0.36 | −0.36 |
| GATA6 | −0.49 | −0.15 | −0.10 | 0.31 | −0.08 | −0.01 | −0.20 | −0.60 | −0.30 | −0.65 | −1.04 |
| GATM | −0.91 | −0.33 | −0.72 | 0.09 | −0.37 | −0.33 | −0.46 | 0.19 | 0.03 | −0.42 | 0.15 |
| GBA | 0.14 | −0.11 | 0.36 | −0.11 | −0.19 | −0.18 | −0.36 | 0.03 | −0.01 | −1.35 | −0.10 |
| GBE1 | −0.47 | −0.05 | 0.19 | 0.01 | −0.20 | −0.09 | 0.62 | −0.38 | −0.05 | −0.21 | 0.98 |
| GCH1 | −0.65 | −0.14 | −0.14 | −0.33 | −0.27 | −0.19 | −0.10 | 0.85 | −0.07 | −0.15 | −0.57 |
| GCKR | 0.42 | 0.32 | 0.74 | −0.03 | 0.29 | 0.21 | −0.40 | 0.04 | −0.09 | −0.70 | 1.55 |
| GCLC | −0.60 | −0.13 | −0.17 | −0.02 | −0.18 | −0.12 | 0.26 | 0.14 | −0.06 | −0.22 | 1.20 |
| GOSH | −0.07 | −0.27 | −0.75 | −0.25 | −0.17 | 0.14 | −0.32 | −0.60 | −0.65 | −0.14 | −0.86 |
| GGCX | −0.67 | −0.06 | −0.31 | 0.07 | 0.10 | 0.00 | −1.01 | 0.58 | 0.13 | −1.08 | −0.02 |
| GHR | −1.85 | −0.41 | −0.90 | −0.20 | −0.27 | −0.40 | −1.33 | 0.58 | 0.07 | −1.23 | 0.41 |
| GJB2 | −0.88 | −0.11 | −0.28 | −0.29 | −0.37 | −0.34 | −0.26 | 0.25 | −0.03 | −0.63 | −0.67 |
| GLDC | −0.31 | −0.11 | −0.76 | −0.30 | 0.02 | −0.20 | −1.67 | −0.33 | −0.42 | −2.07 | −0.84 |
| GLIS3 | −0.57 | 0.03 | −0.19 | 0.00 | −0.13 | −0.05 | −0.52 | −0.49 | 0.50 | 0.06 | 1.10 |
| GLS2 | −0.29 | −0.07 | −0.29 | −0.39 | −0.24 | −0.23 | −0.51 | 0.28 | −0.16 | −0.28 | 0.29 |
| GLYCTK | −0.78 | 0.01 | −0.20 | 0.22 | 0.32 | 0.18 | −0.29 | 0.73 | −0.20 | 0.54 | 0.79 |
| GNE | −0.38 | −0.13 | 0.16 | −0.06 | −0.21 | −0.10 | 0.10 | 0.54 | 0.12 | −0.30 | 1.29 |
| GNPTAB | −0.68 | −0.30 | −0.33 | −0.40 | −0.49 | −0.39 | −0.67 | 0.39 | 0.46 | −0.78 | 0.78 |
| GPC3 | 0.27 | −0.07 | −0.01 | −0.42 | −0.45 | −0.40 | −0.46 | 0.25 | −0.02 | 0.04 | 0.04 |
| GPD1 | 0.74 | −0.04 | −0.60 | 0.29 | 0.37 | 0.37 | −0.80 | 0.33 | −0.11 | −1.12 | −0.49 |
| GPHN | 0.53 | 0.02 | −0.04 | −0.10 | −0.14 | −0.08 | −0.41 | 0.50 | 0.25 | −0.43 | 0.59 |
| GTPBP3 | 0.19 | 0.22 | 0.06 | 0.23 | 0.32 | 0.29 | −0.20 | 0.13 | 0.03 | −0.59 | −0.97 |
| GUSB | −0.23 | −0.29 | −0.12 | −0.38 | −0.26 | −0.38 | −0.27 | −0.01 | −0.27 | −0.01 | −0.34 |
| GYG1 | −0.31 | −0.28 | −0.35 | −0.40 | −0.32 | −0.28 | −0.03 | −0.42 | −0.35 | −0.67 | −0.45 |
| GYS1 | 0.48 | 0.09 | −0.03 | 0.03 | 0.05 | 0.08 | −0.02 | −0.21 | 0.09 | −1.87 | −0.46 |
| GYS2 | −0.17 | −0.20 | 0.22 | 0.19 | −0.03 | −0.07 | −0.89 | 1.58 | −0.51 | −0.11 | 1.21 |
| H19 | −0.18 | −0.23 | −0.29 | −0.15 | −0.21 | −0.19 | −0.20 | 0.35 | −0.15 | −0.15 | 0.75 |
| H6PD | −1.53 | −0.01 | −0.40 | 0.44 | 0.18 | −0.06 | −0.81 | 0.32 | 0.45 | −0.62 | −1.20 |
| HAL | −2.10 | −0.41 | −0.62 | −0.71 | −0.09 | −0.42 | −1.21 | 0.81 | −0.14 | −0.16 | −0.24 |
| HAX1 | 0.05 | −0.26 | −0.01 | −0.17 | −0.12 | −0.14 | 0.19 | −0.13 | −0.33 | −0.07 | −0.09 |
| HBA1 | 1.42 | 0.00 | 0.00 | 1.10 | 1.07 | 0.57 | 0.89 | −0.98 | −0.94 | 0.98 | −1.43 |
| HBB | 1.60 | −0.56 | 0.56 | 0.21 | 0.07 | 1.13 | 1.94 | 0.56 | 0.48 | 1.33 | 0.75 |
| HGD | −0.76 | 0.04 | −0.90 | 0.03 | −0.10 | −0.14 | −0.60 | 2.00 | −0.17 | −1.31 | 1.09 |
| HLA-B | −0.24 | −0.33 | −0.03 | 0.03 | 0.05 | −0.09 | −0.12 | −0.45 | −0.39 | −0.27 | −1.01 |
| HMBS | −0.30 | −0.06 | −0.27 | −0.19 | −0.09 | −0.02 | −0.06 | 0.02 | −0.48 | −0.34 | −0.58 |
| HMGCR | 0.35 | −0.03 | 1.05 | −0.08 | −0.11 | −0.18 | 0.59 | 0.06 | 0.12 | 0.31 | 2.15 |
| HMGCS2 | −2.07 | −0.40 | −0.19 | 0.70 | −0.21 | −0.49 | 0.71 | 3.80 | −0.67 | 0.24 | 1.75 |
| HMOX1 | 0.49 | −0.04 | −0.11 | 0.65 | 0.22 | −0.56 | 0.45 | −0.38 | −0.04 | −0.12 | 2.76 |
| HNF1A | −0.27 | 0.17 | −0.10 | 0.20 | 0.33 | 0.19 | −0.11 | 0.28 | −0.16 | −0.30 | 0.44 |
| HNF1B | −1.15 | −0.03 | −0.05 | −0.12 | −0.02 | −0.21 | −0.64 | −0.34 | −0.12 | −0.60 | −0.26 |
| HNF4A | −0.51 | −0.04 | −0.14 | 0.55 | 0.30 | 0.23 | −0.66 | 0.50 | 0.19 | −0.60 | 0.93 |
| HOGA1 | −0.21 | 0.27 | 0.08 | 0.15 | 0.80 | 0.38 | −0.63 | −0.04 | 0.27 | −0.72 | 1.16 |
| HPD | −0.37 | −0.02 | −0.18 | −0.05 | 0.10 | 0.05 | −0.77 | −0.10 | −0.35 | −0.74 | 0.50 |
| HPGD | −1.62 | −0.47 | −0.76 | −0.17 | −0.69 | −0.53 | −0.52 | 0.86 | −0.40 | −0.54 | −0.26 |
| HPS4 | 0.55 | 0.06 | 0.26 | −0.07 | −0.04 | −0.08 | 0.12 | 0.12 | 0.29 | 0.03 | 0.35 |
| HPS5 | 0.02 | 0.05 | 0.12 | −0.15 | −0.12 | −0.02 | −0.54 | 0.51 | 0.35 | −0.72 | 0.65 |
| HPS6 | −0.52 | −0.03 | −0.01 | 0.09 | 0.31 | 0.26 | −0.30 | −0.25 | −0.41 | −0.94 | −1.21 |
| HPX | −0.12 | −0.27 | −0.10 | −0.07 | −0.17 | −0.23 | −0.21 | 0.14 | −0.30 | 0.18 | −0.02 |
| HRAS | −0.26 | −0.22 | −0.12 | −0.24 | −0.15 | −0.08 | 0.63 | −0.45 | −0.56 | 0.21 | −1.10 |
| HRSP12 | −0.86 | −0.52 | −0.83 | −0.42 | −0.47 | −0.37 | −0.37 | 0.39 | −0.54 | 0.32 | −0.05 |
| HSD17B10 | −0.26 | −0.25 | −0.29 | −0.20 | −0.09 | −0.10 | 0.22 | −0.54 | −0.70 | −0.09 | −0.91 |
| HSD3B7 | 0.20 | −0.11 | 0.30 | −0.11 | −0.05 | −0.04 | 0.05 | −0.18 | −0.03 | −0.35 | −1.24 |
| HTT | −0.02 | 0.02 | −0.04 | 0.01 | 0.02 | 0.05 | −0.55 | 0.16 | 0.39 | −1.06 | 0.18 |
| HYAL1 | −0.04 | −0.14 | −0.17 | −0.27 | −0.20 | −0.25 | −0.29 | 0.36 | −0.55 | −1.00 | −0.52 |
| HYLS1 | −0.58 | −0.12 | −0.34 | −0.39 | −0.60 | −0.30 | −0.23 | −0.24 | −0.33 | −0.80 | 0.10 |
| ID1 | −2.97 | −0.59 | −1.26 | −0.69 | 0.13 | −0.28 | −2.79 | −2.43 | −1.77 | −4.50 | −5.82 |
| IDH1 | −0.52 | −0.14 | 0.27 | −0.16 | −0.29 | −0.28 | 0.32 | 0.29 | −0.09 | 0.86 | 1.43 |
| IDH2 | −1.22 | −0.32 | −0.54 | 0.09 | −0.11 | −0.21 | −0.60 | −0.30 | −0.23 | −0.74 | −0.29 |
| IDUA | 0.56 | 0.19 | 0.34 | 0.14 | 0.23 | 0.15 | 0.44 | −0.10 | −0.32 | 0.20 | −1.63 |
| IFIH1 | −0.52 | −0.21 | −0.45 | −0.12 | −0.40 | −0.32 | −0.59 | 0.01 | 0.13 | −0.50 | −0.56 |
| IFNAR2 | −1.04 | −0.29 | −0.45 | −0.17 | −0.51 | −0.42 | −0.75 | −0.43 | 0.19 | −0.84 | −1.19 |
| IFT122 | −0.32 | 0.10 | −0.36 | −0.12 | −0.15 | −0.11 | −0.72 | −0.10 | −0.12 | −1.01 | −0.64 |
| IFT140 | 0.00 | 0.43 | 0.10 | 0.34 | 0.44 | 0.33 | −0.30 | 0.44 | 0.29 | −0.77 | −0.22 |
| IFT172 | −0.21 | 0.14 | −0.12 | 0.23 | 0.36 | 0.13 | −0.29 | 0.39 | 0.26 | −0.19 | 0.51 |
| IFT27 | −0.56 | −0.27 | −0.35 | 0.02 | −0.11 | −0.18 | −0.22 | −0.19 | −0.54 | 0.19 | −0.71 |
| IFT80 | −0.78 | 0.04 | −0.19 | −0.09 | 0.00 | 0.06 | −0.43 | 0.09 | 0.31 | −1.43 | 0.14 |
| IGFALS | −0.06 | −0.06 | −0.34 | 0.31 | 0.33 | 0.23 | −0.13 | 0.03 | 0.02 | 0.15 | −1.44 |
| IKBKG | 0.11 | 0.24 | 0.02 | 0.33 | 0.48 | 0.40 | −0.19 | −0.65 | 0.00 | −0.80 | 1.04 |
| IL1RN | −0.94 | 0.06 | 1.46 | −0.16 | −0.20 | −0.24 | −0.75 | −0.34 | −0.08 | 1.00 | 0.22 |
| IL2RG | 0.13 | −0.28 | −0.69 | −0.88 | −0.08 | −0.39 | −0.34 | −0.32 | −0.13 | −0.78 | 0.15 |
| INSR | −0.54 | −0.06 | 0.62 | 0.13 | 0.01 | −0.14 | 0.13 | 0.52 | 0.51 | 0.16 | 1.53 |
| INVS | 0.04 | −0.01 | −0.13 | 0.00 | −0.15 | −0.20 | −0.40 | 0.10 | 0.24 | −0.69 | 0.60 |
| ISYNA1 | −0.21 | 0.20 | 0.44 | 0.19 | 0.20 | 0.22 | −0.26 | −0.19 | −0.39 | −1.04 | −0.32 |
| ITGB3 | −0.64 | 0.13 | 0.52 | 0.48 | 0.02 | 0.03 | 0.10 | −1.16 | 0.95 | −0.60 | 0.77 |
| IYD | −1.08 | −0.12 | −0.25 | 0.02 | −0.15 | −0.05 | −0.56 | 0.46 | 0.48 | −0.18 | 1.41 |
| JAG1 | 0.89 | 0.14 | 0.13 | 0.26 | 0.14 | 0.07 | 0.00 | −0.86 | 0.68 | 0.27 | 0.33 |
| JAK2 | 0.01 | 0.02 | 0.24 | −0.40 | −0.26 | −0.06 | −0.01 | 0.22 | 0.54 | 0.38 | 0.31 |
| KCTD11 | −0.21 | 0.08 | 0.07 | 0.40 | 0.53 | 0.41 | −0.60 | −0.16 | −0.11 | −1.26 | −1.21 |
| KCTD7 | 0.84 | 0.01 | 0.41 | 0.27 | 0.26 | 0.20 | 0.11 | 0.60 | 0.70 | 0.10 | 1.22 |
| KHK | −0.09 | −0.22 | −0.44 | 0.01 | 0.13 | 0.03 | −0.13 | 0.44 | −0.38 | −0.18 | −0.33 |
| KIAA0586 | −0.01 | −0.06 | −0.17 | −0.06 | −0.32 | −0.11 | −0.75 | 0.06 | 0.60 | −1.05 | −0.06 |
| KMT2D | 0.44 | 0.45 | 0.42 | 0.46 | 0.57 | 0.55 | −0.35 | 0.27 | 1.06 | −0.82 | 1.28 |
| KPTN | −0.48 | −0.07 | −0.07 | 0.12 | 0.03 | 0.27 | 0.28 | 0.04 | −0.38 | −0.50 | −0.37 |
| KRIT1 | −0.04 | −0.04 | 0.01 | −0.16 | −0.32 | −0.22 | −0.17 | 0.11 | 0.41 | −0.26 | 0.49 |
| KRT18 | −1.11 | −0.03 | −0.13 | −0.14 | 0.14 | 0.11 | −0.82 | −0.93 | −0.36 | −1.64 | −0.86 |
| KYNU | −1.31 | 0.10 | 0.17 | −0.54 | −0.28 | −0.08 | −0.44 | −0.75 | −0.37 | −1.45 | 0.03 |
| LAMB2 | −0.50 | 0.02 | 0.06 | 0.11 | 0.14 | 0.05 | −0.44 | −0.04 | 0.06 | −1.40 | −0.78 |
| LCAT | −0.38 | −0.02 | −0.28 | 0.06 | 0.18 | 0.01 | −0.50 | −0.06 | −0.37 | −0.79 | −0.55 |
| LDHB | 0.24 | 0.12 | −0.18 | −0.30 | −0.23 | −0.02 | −0.79 | −0.73 | −0.65 | −1.06 | 0.89 |
| LDLR | 0.11 | 0.22 | 2.08 | 0.00 | 0.23 | 0.04 | 1.18 | −0.09 | 0.30 | 0.74 | 1.45 |
| LDLRAP1 | −0.60 | 0.02 | −0.07 | −0.06 | 0.03 | 0.06 | −0.44 | −0.14 | −0.21 | −0.86 | −0.28 |
| LIPC | −1.95 | −0.23 | −0.87 | −0.46 | −0.23 | −0.34 | −1.77 | −0.03 | −0.13 | −1.85 | −0.55 |
| LIPT1 | −0.53 | −0.39 | −0.56 | −0.69 | −0.63 | −0.42 | −0.13 | −0.04 | −0.54 | 0.18 | −0.47 |
| LMNB2 | 0.11 | 0.10 | −0.21 | 0.18 | 0.36 | 0.37 | −0.52 | −0.40 | 0.11 | −1.06 | −0.36 |
| LPA | −0.19 | −0.04 | −0.04 | −0.20 | 0.04 | −0.35 | −0.37 | 0.20 | −0.25 | −0.08 | 0.19 |
| LPIN1 | 0.64 | 0.04 | 0.99 | 0.04 | −0.08 | −0.03 | 1.16 | −0.08 | 0.22 | 0.94 | 1.44 |
| LPIN2 | −0.95 | −0.01 | 0.59 | 0.28 | 0.08 | −0.03 | −0.01 | 1.74 | 0.49 | 0.25 | 0.01 |
| LRBA | −0.17 | −0.07 | −0.12 | −0.20 | −0.14 | −0.17 | −0.85 | 0.51 | 0.75 | −0.88 | 0.75 |
| LRP5 | −0.10 | 0.04 | −0.15 | 0.08 | 0.14 | 0.04 | −0.64 | 0.06 | 0.43 | −0.96 | 0.46 |
| LYRM4 | −0.40 | −0.37 | −0.29 | −0.38 | −0.40 | −0.39 | −0.03 | −0.60 | −0.55 | −0.19 | −0.40 |
| LYST | 0.84 | 0.18 | 0.54 | 0.09 | −0.05 | 0.08 | 0.02 | 0.12 | 0.64 | −0.21 | 0.36 |
| MAN1A1 | −0.16 | −0.28 | −0.15 | −0.26 | −0.36 | −0.31 | −0.52 | 0.60 | 0.14 | −0.79 | 0.56 |
| MAN2B1 | −0.06 | −0.01 | −0.02 | 0.06 | 0.14 | 0.08 | −0.49 | −0.01 | −0.04 | −1.04 | −0.35 |
| MAOB | −0.74 | −0.25 | −0.46 | 0.03 | −0.07 | −0.13 | −0.47 | 0.54 | 0.17 | −0.30 | 0.74 |
| MAT1A | 0.40 | 0.12 | 0.27 | 0.40 | 0.14 | 0.05 | 0.41 | 0.58 | 0.08 | 0.70 | 0.84 |
| MBL2 | −1.09 | −0.20 | −1.05 | −0.16 | −0.25 | −0.31 | −1.30 | 1.22 | 0.14 | −0.65 | 1.47 |
| MCCC1 | 0.23 | −0.13 | 0.13 | −0.29 | −0.27 | −0.20 | −0.28 | 0.08 | 0.11 | −0.75 | 0.44 |
| MCCC2 | −0.40 | −0.18 | −0.40 | −0.35 | −0.31 | −0.22 | −0.77 | 0.18 | 0.18 | −1.20 | 0.25 |
| MCM6 | −0.34 | −0.17 | −0.29 | −0.47 | −0.34 | −0.17 | −0.77 | −0.26 | −0.06 | −0.77 | −0.09 |
| MEN1 | −0.06 | 0.14 | 0.11 | 0.04 | 0.18 | 0.21 | −0.45 | 0.00 | 0.15 | −1.14 | −0.09 |
| MET | −0.53 | −0.15 | 0.03 | −0.67 | −0.27 | −0.24 | −1.00 | −0.47 | 0.38 | −0.72 | −0.01 |
| MGAT2 | −0.41 | −0.17 | −0.21 | 0.06 | −0.09 | −0.05 | −0.13 | 0.29 | −0.07 | 0.16 | −1.16 |
| MKS1 | −0.16 | −0.15 | −0.22 | −0.02 | 0.23 | 0.08 | −0.21 | −0.24 | −0.06 | −0.57 | −0.33 |
| MLH1 | −0.22 | −0.16 | −0.19 | −0.14 | −0.18 | −0.05 | −0.18 | 0.15 | −0.06 | −0.64 | 0.43 |
| MMAA | −1.07 | −0.08 | −0.32 | −0.21 | −0.25 | −0.15 | −0.50 | 0.05 | −0.15 | −0.70 | −0.40 |
| MMAB | 0.81 | −0.16 | 0.20 | −0.09 | −0.09 | −0.01 | 0.90 | −0.02 | −0.41 | 1.11 | 0.85 |
| MMADHC | −0.93 | −0.32 | −0.50 | −0.47 | −0.38 | −0.26 | −0.18 | 0.01 | −0.23 | −0.40 | 0.34 |
| MOCS1 | 0.23 | 0.01 | 0.23 | −0.30 | 0.03 | 0.09 | −0.08 | 0.36 | −0.05 | −0.51 | 1.01 |
| MOGAT2 | −0.69 | 0.28 | −0.61 | 0.85 | 0.66 | 0.41 | −1.21 | 0.31 | 0.60 | −1.01 | 0.29 |
| MOGS | 0.15 | 0.06 | 0.11 | 0.07 | 0.33 | 0.27 | −0.60 | −0.10 | −0.09 | −1.05 | −0.60 |
| MPZ | −0.09 | 0.00 | 0.07 | −0.05 | 0.37 | 0.18 | −0.56 | −0.86 | −0.24 | −1.00 | 1.76 |
| MRPL3 | −0.23 | −0.23 | −0.53 | −0.52 | −0.47 | −0.31 | −0.62 | −0.23 | −0.27 | −0.99 | −0.09 |
| MTHFR | 0.23 | 0.25 | 0.65 | 0.31 | 0.37 | 0.40 | 0.49 | 0.49 | 0.20 | 0.03 | 0.45 |
| MTM1 | −0.26 | −0.12 | 0.12 | −0.06 | −0.29 | −0.09 | −0.02 | 0.08 | 0.08 | −0.29 | 0.41 |
| MTO1 | 0.09 | −0.04 | −0.09 | −0.07 | −0.13 | −0.04 | 0.04 | 0.20 | −0.07 | −0.24 | 0.36 |
| MTR | 0.20 | 0.16 | 0.16 | 0.07 | 0.10 | 0.08 | −0.39 | 0.45 | 0.67 | −0.48 | 0.87 |
| MTRR | −0.27 | 0.01 | −0.21 | −0.33 | −0.43 | −0.17 | −0.47 | 0.00 | 0.08 | −0.78 | 0.21 |
| MTTP | −1.98 | −0.24 | −0.64 | 0.43 | −0.18 | −0.23 | −1.42 | 0.15 | −0.17 | −1.15 | 0.43 |
| MVK | 1.16 | −0.07 | 0.79 | 0.07 | 0.07 | 0.10 | 1.22 | 0.10 | −0.08 | 0.45 | 1.56 |
| MYH9 | 0.18 | 0.09 | −0.08 | 0.33 | 0.20 | 0.18 | −0.33 | −0.33 | 0.58 | −0.73 | −0.08 |
| MYO5B | 0.02 | −0.06 | 0.59 | −0.05 | −0.08 | −0.06 | −0.29 | −0.43 | 0.33 | −0.05 | 0.62 |
| NAGS | 1.90 | 0.26 | 1.08 | 0.21 | 0.31 | 0.21 | 0.01 | 0.20 | −0.32 | 0.70 | 0.26 |
| NARS2 | −0.36 | −0.20 | −0.42 | −0.41 | −0.42 | −0.27 | −0.49 | 0.00 | −0.03 | −0.50 | −0.19 |
| NAT2 | −0.49 | −0.29 | −0.41 | −0.29 | −0.40 | −0.32 | −0.48 | 0.08 | −0.59 | −0.81 | −0.27 |
| NBAS | −0.17 | −0.13 | −0.22 | −0.16 | −0.22 | −0.15 | −0.59 | 0.29 | 0.46 | −0.63 | 0.73 |
| NCF2 | −0.41 | 0.02 | 0.36 | 0.22 | −0.20 | 0.13 | 0.09 | −3.37 | −0.18 | −0.57 | 0.26 |
| NEK1 | 0.03 | −0.16 | 0.14 | −0.06 | −0.22 | −0.15 | 0.04 | −0.17 | 0.16 | 0.22 | 0.17 |
| NEK8 | 0.38 | 0.23 | 0.30 | 0.03 | 0.47 | 0.26 | 0.00 | 0.46 | 0.19 | −0.62 | 0.11 |
| NEU1 | 0.91 | −0.31 | 0.86 | −0.03 | −0.25 | −0.22 | 0.59 | 0.08 | −0.20 | −0.05 | −0.17 |
| NFKB2 | 0.10 | 0.11 | −0.05 | 0.25 | 0.30 | 0.35 | −0.12 | 0.55 | −0.02 | −0.94 | −2.33 |
| NFKBIA | −0.26 | −0.18 | −0.16 | −0.11 | −0.19 | −0.22 | −0.30 | 0.19 | −0.28 | −0.35 | −3.28 |
| NFU1 | −0.73 | −0.27 | −0.28 | −0.40 | −0.52 | −0.32 | −0.03 | −0.28 | −0.43 | 0.00 | −0.48 |
| NGLY1 | −0.23 | −0.23 | −0.15 | −0.30 | −0.40 | −0.21 | −0.15 | −0.11 | −0.15 | −0.34 | 0.06 |
| NHP2 | 0.25 | 0.17 | 0.13 | 0.31 | 0.65 | 0.57 | 0.09 | −0.46 | −0.58 | −0.27 | −0.90 |
| NME1 | 0.01 | −0.23 | −0.48 | −0.31 | 0.00 | 0.01 | −0.15 | −0.68 | −0.57 | −0.49 | −0.91 |
| NOD2 | 1.83 | 0.14 | 1.20 | 0.50 | 0.30 | 0.12 | 0.63 | −1.05 | 0.28 | 0.45 | −1.01 |
| NOP10 | 0.12 | −0.09 | 0.18 | −0.07 | 0.03 | 0.11 | −0.03 | −0.53 | −0.73 | −0.33 | −1.02 |
| NOTCH1 | 0.24 | 0.28 | 0.27 | 0.26 | 0.23 | 0.28 | −0.36 | −0.17 | 0.43 | −0.48 | 0.43 |
| NOTCH2 | −0.38 | 0.02 | 0.33 | 0.23 | 0.01 | 0.05 | −0.75 | −0.18 | 0.74 | −1.02 | 0.31 |
| NPC1 | 0.82 | 0.03 | 1.25 | 0.02 | −0.09 | −0.09 | 0.01 | 0.19 | 0.35 | −0.25 | 1.02 |
| NR1H4 | 0.95 | −0.25 | 1.61 | 0.02 | 0.00 | −0.19 | 0.99 | 1.02 | 0.02 | 1.01 | 1.37 |
| NUBPL | −0.36 | −0.30 | −0.54 | −0.32 | −0.11 | −0.17 | −0.64 | 0.01 | 0.16 | −0.35 | 0.25 |
| NUDT12 | −0.02 | −0.22 | −0.15 | −0.32 | −0.32 | −0.31 | 0.20 | 0.31 | 0.03 | 0.11 | 0.36 |
| NUDT7 | −1.48 | −0.56 | −0.69 | −0.44 | −0.70 | −0.65 | −0.47 | 0.65 | −0.47 | 0.05 | −1.30 |
| OAT | −0.86 | −0.20 | −0.27 | −0.49 | −0.40 | −0.33 | −0.06 | 0.65 | −0.21 | −0.04 | −0.12 |
| OPA3 | 0.01 | −0.11 | 0.06 | 0.13 | 0.14 | 0.04 | −0.08 | 0.30 | 0.12 | −0.61 | −0.04 |
| OTC | −0.94 | −0.50 | −0.84 | −0.22 | −0.33 | −0.40 | −0.62 | 0.47 | −0.26 | 0.27 | 0.59 |
| PAF1 | −0.48 | −0.12 | −0.04 | −0.06 | −0.04 | 0.01 | 0.01 | −0.30 | −0.45 | −0.86 | −0.09 |
| PAH | 1.19 | −0.15 | 0.65 | −0.07 | −0.22 | −0.30 | 0.47 | 2.09 | 0.17 | −0.23 | 1.89 |
| PANK2 | −0.05 | −0.12 | 0.04 | −0.03 | −0.22 | −0.09 | −0.16 | −0.06 | −0.18 | −0.21 | −0.21 |
| PAOX | −0.55 | −0.49 | −0.01 | −0.01 | −0.04 | −0.17 | −0.11 | 0.01 | −0.38 | −0.59 | −1.45 |
| PC | −0.34 | 0.11 | 0.13 | −0.01 | 0.27 | 0.17 | −0.71 | 0.51 | 0.12 | −0.87 | 0.77 |
| PCCA | −0.12 | −0.37 | 0.20 | −0.34 | −0.23 | −0.33 | −0.22 | 0.01 | 0.08 | 0.18 | 0.23 |
| PCK2 | 0.48 | −0.09 | 0.48 | 0.36 | 0.18 | 0.06 | 0.61 | −0.02 | −0.23 | 1.10 | −0.48 |
| PCSK9 | 0.90 | 0.08 | 1.41 | 0.28 | 0.46 | 0.14 | 0.99 | 0.29 | 0.00 | 0.75 | 2.33 |
| PCYT1A | −0.01 | 0.05 | −0.05 | −0.02 | −0.07 | 0.05 | −0.33 | −0.16 | 0.30 | −1.43 | 0.53 |
| PDGFRA | −1.50 | −0.29 | −0.23 | −0.20 | −0.06 | −0.27 | −1.74 | −0.81 | −0.10 | −1.29 | −1.12 |
| PDP1 | 0.23 | −0.25 | 0.52 | −0.15 | −0.30 | −0.11 | 0.12 | −0.34 | 0.53 | 0.04 | −0.25 |
| PEPD | 0.33 | −0.20 | 0.48 | 0.02 | 0.03 | −0.03 | 0.13 | 0.66 | −0.14 | −0.35 | −0.15 |
| PEX11A | −0.32 | −0.15 | −0.28 | −0.03 | −0.27 | −0.31 | 0.18 | 0.43 | −0.41 | −0.17 | 0.50 |
| PEX12 | 0.11 | −0.22 | −0.20 | −0.29 | −0.20 | −0.20 | 0.01 | 0.13 | 0.05 | 0.13 | 0.33 |
| PEX14 | 0.08 | −0.02 | 0.00 | −0.03 | 0.19 | 0.13 | −0.33 | 0.04 | −0.13 | −0.69 | 0.38 |
| PFKM | 0.00 | 0.23 | −0.23 | 0.14 | 0.45 | 0.52 | −1.25 | 0.25 | −0.23 | −1.66 | 0.12 |
| PGM1 | −1.00 | −0.16 | −0.16 | −0.09 | −0.24 | −0.21 | −0.48 | 0.47 | −0.13 | −0.64 | 0.18 |
| PHGDH | 0.64 | −0.05 | 0.39 | 0.52 | 0.38 | 0.16 | 0.62 | 0.06 | −0.11 | 1.54 | 1.33 |
| PHKA1 | 0.80 | −0.01 | 0.47 | −0.04 | −0.18 | −0.12 | 0.03 | 0.48 | 0.10 | −0.16 | 1.27 |
| PHKA2 | −0.34 | 0.15 | −0.10 | 0.05 | 0.11 | 0.03 | −0.41 | 0.42 | 0.13 | −0.57 | 0.30 |
| PHKB | −0.47 | −0.22 | −0.14 | −0.26 | −0.39 | −0.31 | −0.25 | −0.11 | 0.24 | −0.45 | 0.52 |
| PHYH | −0.65 | −0.31 | −0.16 | −0.27 | −0.26 | −0.25 | −0.19 | 0.21 | −0.32 | 0.12 | 0.08 |
| PIEZO1 | −0.15 | 0.15 | −0.14 | 0.58 | 0.30 | 0.24 | −0.53 | −0.77 | 0.41 | −0.76 | −1.07 |
| PIGA | −0.72 | −0.16 | 0.52 | −0.26 | −0.37 | −0.18 | 0.50 | −0.10 | −0.11 | 1.36 | 0.14 |
| PIGM | −0.06 | −0.07 | −0.39 | −0.29 | −0.35 | −0.22 | −0.27 | −0.06 | −0.08 | −0.80 | −0.83 |
| PKD1 | 0.73 | 0.15 | 0.49 | 0.57 | 0.61 | 0.39 | 0.15 | 0.25 | 0.39 | 0.05 | 0.32 |
| PKD2 | 0.02 | 0.37 | 0.29 | 0.22 | 0.28 | 0.28 | −0.62 | −0.01 | 0.49 | −1.14 | 0.71 |
| PKHD1 | −2.34 | 0.19 | −0.92 | −0.23 | 0.34 | 0.13 | −1.31 | −0.57 | 0.74 | −1.45 | 0.49 |
| PKLR | −0.17 | −0.16 | −0.20 | 0.52 | 0.35 | 0.18 | −0.59 | 1.28 | −0.07 | 0.11 | 0.65 |
| PLA2G6 | −1.06 | 0.51 | 0.42 | 0.63 | 0.59 | 0.39 | −0.40 | 0.32 | 0.07 | −0.67 | 0.25 |
| PLEKHM1 | 1.11 | 0.04 | 1.28 | −0.07 | 0.10 | 0.12 | 0.70 | −0.15 | 0.01 | −0.28 | 0.46 |
| PMVK | −0.10 | −0.16 | −0.21 | 0.06 | 0.39 | 0.32 | 0.48 | −0.33 | −0.63 | 0.53 | −1.31 |
| PNP | −0.13 | −0.23 | −0.17 | −0.39 | −0.40 | −0.27 | −0.44 | −0.35 | −0.18 | −1.35 | −0.53 |
| PNPLA2 | −1.42 | 0.05 | 0.07 | 0.33 | 0.02 | −0.06 | −0.11 | 0.16 | −0.20 | −0.71 | −0.32 |
| POLD1 | 0.17 | 0.08 | −0.12 | −0.05 | 0.30 | 0.28 | −0.29 | 0.09 | −0.13 | −1.16 | −0.37 |
| POLG | −0.13 | −0.07 | −0.03 | −0.03 | 0.10 | 0.10 | −0.43 | 0.15 | 0.16 | −1.19 | 0.06 |
| POLG2 | 0.40 | 0.18 | −0.14 | 0.04 | 0.22 | 0.27 | 0.18 | −0.08 | −0.33 | 0.02 | −0.78 |
| POR | 0.97 | 0.10 | 0.43 | 0.40 | 0.16 | 0.03 | 0.31 | 1.21 | 0.28 | 0.50 | 0.97 |
| PPAPDC2 | −0.42 | −0.13 | −0.29 | −0.26 | −0.14 | −0.09 | −0.29 | −0.05 | 0.10 | −0.85 | −0.56 |
| PPARA | −0.10 | 0.03 | 0.22 | −0.06 | −0.14 | −0.11 | −0.21 | 0.68 | 0.61 | 0.25 | 2.22 |
| PPARG | −1.02 | −0.26 | 0.06 | −0.84 | −0.47 | −0.55 | −0.56 | −1.28 | −0.55 | 0.12 | −0.62 |
| PPM1K | −0.38 | −0.05 | −0.25 | −0.34 | −0.24 | −0.18 | −0.30 | −0.12 | −0.14 | −0.51 | 0.51 |
| PRKCD | −0.05 | −0.12 | 0.35 | 0.27 | 0.06 | 0.07 | −0.02 | −0.04 | 0.01 | −0.74 | −0.21 |
| PRODH | −0.25 | −0.39 | −0.28 | −0.19 | −0.14 | −0.29 | −0.15 | 4.62 | 0.61 | 0.60 | 0.07 |
| PROS1 | −0.44 | −0.24 | −0.01 | −0.07 | −0.20 | −0.18 | −0.77 | 0.16 | 0.17 | −1.07 | 0.33 |
| PROZ | −0.77 | −0.19 | 0.10 | 0.30 | 0.10 | −0.02 | −0.15 | 0.61 | 0.11 | −0.39 | 0.15 |
| PSAT1 | 0.47 | −0.19 | 0.10 | −0.09 | −0.18 | −0.15 | 0.69 | −0.50 | −0.08 | 1.32 | 1.24 |
| PSMB8 | −0.36 | −0.30 | −0.49 | −0.08 | 0.08 | −0.03 | 0.06 | −0.60 | −0.52 | 0.34 | −1.21 |
| PSPH | 0.40 | −0.23 | 0.16 | −0.21 | −0.12 | −0.17 | 0.25 | 0.04 | 0.09 | 0.54 | 0.17 |
| PTCH1 | −1.05 | 0.17 | 0.28 | 0.04 | 0.20 | −0.09 | −0.78 | −0.30 | 0.53 | −0.94 | 0.06 |
| PTRF | −0.89 | −0.06 | −0.21 | 0.03 | 0.25 | 0.29 | −0.01 | −0.83 | −0.08 | −1.22 | −1.09 |
| PTRH2 | −0.07 | −0.23 | −0.32 | −0.55 | −0.47 | −0.35 | −0.09 | −0.60 | −0.58 | −0.48 | −0.93 |
| PUS1 | 0.57 | 0.11 | 0.09 | 0.05 | 0.29 | 0.21 | 0.16 | −0.42 | −0.32 | −0.05 | −1.50 |
| PXMP2 | −0.33 | −0.32 | −0.33 | −0.29 | −0.29 | −0.29 | −0.29 | 0.64 | −0.45 | 0.01 | −0.25 |
| PXMP4 | −0.36 | −0.13 | −0.10 | −0.18 | −0.05 | −0.03 | 0.23 | 0.84 | 0.27 | 0.42 | 1.27 |
| RAI1 | 0.19 | 0.27 | 0.21 | 0.23 | 0.38 | 0.44 | −0.46 | −0.50 | 0.18 | −0.75 | −0.03 |
| RANBP2 | −0.07 | −0.04 | −0.11 | −0.11 | −0.16 | −0.05 | −0.58 | 0.20 | 0.42 | −0.94 | 0.38 |
| RASA1 | −0.42 | −0.15 | −0.12 | −0.21 | −0.26 | −0.23 | −0.14 | −0.04 | 0.28 | 0.12 | 0.73 |
| RBCK1 | 0.94 | 0.11 | 1.08 | 0.27 | 0.26 | 0.18 | 0.65 | −0.31 | −0.05 | 0.59 | 0.37 |
| RBM10 | −0.13 | 0.05 | −0.04 | 0.04 | 0.11 | 0.12 | −0.16 | 0.00 | −0.21 | −1.06 | 0.05 |
| REN | 1.05 | 0.64 | 0.13 | −0.30 | 0.46 | 0.61 | −1.03 | −1.57 | −0.44 | −1.03 | −0.93 |
| RMND1 | −0.11 | −0.27 | −0.36 | −0.58 | −0.47 | −0.28 | −0.40 | 0.28 | −0.07 | −0.25 | 0.11 |
| RMRP | −3.71 | −2.97 | −2.28 | −2.09 | −1.76 | −2.32 | 1.54 | −0.60 | −0.89 | 1.80 | −0.58 |
| RNASEH2A | −0.23 | 0.05 | 0.10 | −0.21 | −0.19 | 0.09 | 0.25 | −0.34 | −0.66 | 0.22 | −0.88 |
| RORC | 1.29 | −0.02 | 0.13 | 0.71 | 0.58 | 0.16 | 0.47 | 2.25 | 0.31 | 1.38 | 3.17 |
| RPGRIP1L | 0.19 | 0.06 | −0.54 | −0.19 | −0.12 | −0.27 | −0.78 | −0.47 | −0.06 | −0.68 | −0.03 |
| RRM2B | −0.08 | −0.28 | −0.49 | −0.30 | −0.34 | −0.21 | −0.75 | 0.45 | 0.48 | −0.32 | 1.19 |
| RSAD1 | 0.26 | −0.02 | −0.13 | −0.06 | 0.11 | 0.12 | 0.13 | 0.22 | −0.11 | 0.01 | −0.23 |
| SAMHD1 | −0.34 | −0.24 | −0.48 | −0.29 | −0.43 | −0.34 | −1.05 | −0.22 | 0.25 | −1.92 | −0.05 |
| SC5D | 0.23 | −0.29 | 0.37 | −0.23 | −0.56 | −0.37 | 0.51 | 0.22 | −0.01 | 0.47 | 1.56 |
| SCARB1 | −0.02 | −0.03 | −0.07 | 0.08 | 0.11 | 0.05 | −0.30 | −0.40 | −0.17 | −0.67 | −0.35 |
| SCN9A | −1.60 | 0.10 | −0.42 | 0.14 | −0.09 | −0.07 | −0.39 | −0.97 | 0.25 | −0.32 | −0.22 |
| SDCCAG8 | 0.08 | −0.06 | −0.06 | −0.25 | −0.10 | −0.12 | −0.30 | 0.11 | −0.03 | −0.50 | 0.11 |
| SEPN1 | −0.51 | −0.11 | −0.10 | −0.10 | −0.02 | 0.00 | −0.85 | −0.18 | 0.28 | −1.37 | −0.07 |
| SERAC1 | −0.28 | −0.22 | −0.02 | −0.29 | −0.34 | −0.07 | −0.14 | −0.05 | 0.29 | −0.31 | 0.26 |
| SERPINA1 | 0.21 | −0.17 | −0.05 | 0.01 | −0.16 | −0.19 | −0.19 | 0.31 | −0.19 | 0.12 | −0.05 |
| SERPING1 | 0.06 | −0.16 | 0.21 | −0.11 | −0.27 | −0.31 | −0.29 | 0.27 | −0.11 | 0.14 | 0.28 |
| SERPINI1 | −0.50 | −0.41 | −0.42 | −0.36 | −0.37 | −0.28 | −0.73 | 0.67 | −0.19 | −0.06 | 0.22 |
| SETBP1 | 0.08 | 0.06 | −0.10 | 0.22 | 0.05 | 0.11 | −0.30 | −0.46 | −0.14 | −0.38 | 0.54 |
| SFXN4 | 0.11 | −0.19 | −0.38 | −0.33 | −0.17 | −0.14 | −0.01 | 0.01 | −0.46 | 0.25 | −0.14 |
| SLC10A1 | −0.60 | −0.31 | −0.68 | 0.00 | −0.14 | −0.40 | −0.88 | 0.97 | −0.03 | −0.16 | 1.13 |
| SLC10A3 | 0.28 | 0.33 | 0.47 | 0.23 | 0.57 | 0.53 | −0.50 | −0.64 | 0.11 | −0.82 | −1.25 |
| SLC10A5 | −0.77 | 0.19 | −0.54 | 0.63 | 0.46 | −0.06 | −0.90 | 1.23 | 0.18 | −1.06 | 2.20 |
| SLC10A7 | −0.46 | −0.38 | −0.22 | −0.35 | −0.54 | −0.46 | −0.52 | −0.12 | 0.23 | −0.12 | 0.55 |
| SLC11A2 | 0.16 | −0.02 | 0.39 | −0.26 | 0.04 | −0.01 | −0.59 | −0.54 | 0.32 | −0.44 | 0.65 |
| SLC19A1 | 0.09 | 0.05 | −0.26 | −0.42 | 0.05 | −0.10 | −0.45 | −0.37 | −0.21 | −0.33 | −0.71 |
| SLC19A2 | 0.28 | 0.00 | 0.54 | 0.22 | −0.17 | −0.05 | −0.03 | −0.01 | 0.32 | −0.15 | 0.62 |
| SLC19A3 | −0.67 | −0.03 | −0.22 | 0.00 | −0.16 | −0.26 | −0.68 | 0.18 | −0.31 | −0.16 | 0.68 |
| SLC22A1 | −0.34 | −0.32 | −0.28 | −0.07 | −0.12 | −0.18 | −0.13 | 0.16 | −0.22 | 0.24 | 0.08 |
| SLC22A5 | 0.26 | 0.05 | −0.23 | 0.32 | −0.16 | 0.03 | 0.06 | −0.46 | −0.06 | −0.38 | −0.72 |
| SLC25A1 | −0.20 | 0.08 | 0.24 | 0.11 | 0.33 | 0.36 | −0.09 | −0.04 | −0.33 | −0.61 | −0.29 |
| SLC25A10 | −0.01 | −0.16 | −0.48 | −0.12 | 0.12 | 0.09 | −0.40 | 0.24 | −0.31 | −0.64 | −0.45 |
| SLC25A12 | 0.10 | 0.23 | −0.22 | −0.11 | 0.27 | 0.10 | −0.41 | −0.27 | −0.27 | −0.97 | −0.06 |
| SLC25A13 | 0.11 | −0.13 | 0.20 | −0.15 | −0.17 | −0.06 | −0.29 | 0.22 | 0.25 | −0.53 | 0.72 |
| SLC25A15 | −0.31 | 0.02 | −0.28 | −0.21 | 0.03 | 0.04 | −0.47 | 1.61 | −0.09 | −0.18 | 0.58 |
| SLC25A19 | −0.01 | −0.15 | 0.03 | −0.35 | −0.19 | −0.02 | 0.26 | 0.07 | −0.43 | −0.47 | −0.70 |
| SLC25A20 | −0.96 | −0.16 | −0.62 | −0.28 | −0.32 | −0.21 | −0.32 | 0.62 | −0.35 | −1.32 | −0.52 |
| SLC25A29 | −0.13 | 0.09 | 0.92 | 0.25 | 0.65 | 0.39 | −0.11 | −0.40 | −0.13 | −0.54 | −1.07 |
| SLC25A32 | 0.18 | −0.05 | −0.21 | −0.44 | −0.32 | −0.19 | −0.13 | −0.06 | −0.10 | −0.47 | −0.50 |
| SLC27A2 | −0.77 | −0.25 | −0.90 | 0.24 | −0.36 | −0.29 | −1.10 | −0.76 | −0.20 | −1.67 | −0.79 |
| SLC27A5 | −0.39 | −0.15 | −0.21 | 0.02 | 0.13 | 0.08 | −0.24 | 0.13 | −0.23 | 0.03 | −0.04 |
| SLC29A3 | −1.50 | −0.27 | −0.39 | −0.01 | −0.02 | −0.12 | −0.87 | −0.38 | −0.06 | −1.19 | 0.25 |
| SLC2A1 | 0.55 | 0.07 | 0.35 | 0.22 | 0.08 | 0.12 | 0.30 | −0.87 | 0.07 | −0.31 | 0.11 |
| SLC2A2 | −1.36 | −0.27 | −0.87 | −1.17 | −0.24 | −0.30 | −3.21 | −0.72 | −0.08 | −3.04 | −0.83 |
| SLC2A9 | 0.11 | −0.08 | 0.44 | 0.09 | 0.16 | 0.47 | −0.07 | 0.69 | 0.54 | 0.66 | 1.37 |
| SLC33A1 | 0.12 | −0.10 | 0.18 | −0.16 | −0.02 | −0.03 | −0.31 | 0.21 | 0.22 | 0.27 | 0.11 |
| SLC35C1 | −0.09 | 0.00 | 0.28 | −0.18 | 0.07 | 0.04 | −0.40 | −0.12 | −0.03 | −1.16 | −0.75 |
| SLC39A4 | 0.35 | 0.26 | 0.41 | 0.06 | 0.32 | 0.24 | −0.01 | −0.40 | −0.86 | −0.13 | −1.05 |
| SLC40A1 | −0.68 | −0.10 | −0.02 | 0.31 | −0.18 | −0.28 | 0.01 | 0.38 | 0.08 | −0.33 | 3.16 |
| SLC46A1 | 0.10 | 0.06 | −0.40 | 0.12 | 0.17 | 0.12 | −0.59 | 0.47 | 0.25 | −0.60 | 0.39 |
| SLC4A4 | 0.05 | −0.03 | −0.18 | −0.43 | −0.30 | −0.25 | −0.88 | 0.06 | 0.43 | −1.02 | 0.92 |
| SLC51A | −1.09 | −0.04 | −0.44 | −0.25 | −0.03 | −0.18 | −0.60 | −0.47 | −0.73 | −0.72 | 0.23 |
| SLC51B | −0.66 | −0.71 | 0.29 | −0.35 | 0.08 | −0.20 | 0.45 | 0.45 | −0.50 | 0.86 | 1.63 |
| SLC6A20 | 0.06 | −0.43 | 0.00 | −0.16 | −0.44 | −0.21 | −0.23 | 0.77 | 0.14 | −1.50 | 0.01 |
| SLC6A6 | 0.08 | 0.19 | 0.52 | 0.17 | −0.29 | 0.11 | 0.29 | −1.63 | 0.23 | −1.49 | 1.47 |
| SLC6A8 | 0.16 | −0.05 | 0.18 | 0.35 | 0.27 | 0.22 | 0.02 | −0.29 | −0.28 | −0.95 | −0.17 |
| SLC7A7 | −0.75 | 0.40 | −0.20 | 0.88 | 0.03 | 0.30 | 0.26 | −1.19 | −0.24 | −0.88 | −1.16 |
| SLCO1B1 | −1.50 | −0.12 | −0.83 | 0.10 | −0.23 | −0.26 | −1.01 | 0.33 | 0.03 | −1.43 | 1.42 |
| SLCO4C1 | 0.08 | −0.01 | 0.73 | 0.09 | −0.05 | −0.38 | 0.24 | 1.18 | −0.12 | 0.39 | 2.16 |
| SMAD4 | −0.48 | −0.15 | −0.12 | −0.09 | −0.17 | −0.15 | −0.03 | 0.10 | 0.05 | 0.23 | 0.49 |
| SMARCB1 | −0.73 | −0.19 | −0.28 | −0.20 | −0.19 | −0.11 | −0.64 | −0.28 | −0.31 | −0.93 | −0.22 |
| SNX10 | −0.75 | −0.21 | 0.26 | −0.57 | −0.57 | −0.43 | −0.26 | −0.06 | 0.11 | 0.06 | −0.20 |
| SORT1 | −0.93 | −0.19 | −0.12 | −0.03 | −0.12 | −0.19 | −0.67 | 0.17 | 0.27 | −1.36 | 0.36 |
| SPP2 | −0.38 | −0.21 | −0.47 | −0.19 | −0.17 | −0.12 | −0.32 | −0.09 | −0.61 | 0.38 | −0.12 |
| SPRTN | 0.00 | −0.02 | −0.33 | −0.18 | −0.18 | 0.06 | −0.35 | −0.04 | 0.09 | −0.59 | −0.07 |
| ST3GAL5 | −0.54 | −0.05 | 0.08 | −0.50 | −0.11 | −0.25 | −0.88 | 0.25 | −0.12 | −0.40 | −0.61 |
| STAT1 | −0.96 | −0.21 | −0.51 | −0.37 | −0.45 | −0.34 | −0.94 | −0.77 | 0.15 | −1.44 | −0.38 |
| STEAP3 | −0.13 | −0.09 | −0.14 | −0.05 | 0.03 | 0.01 | −0.96 | −0.37 | 0.15 | −1.00 | −0.38 |
| STIM1 | −0.35 | −0.13 | 0.02 | −0.12 | −0.07 | −0.11 | −0.61 | 0.20 | 0.05 | −0.67 | 0.60 |
| SULT2A1 | −0.21 | −0.09 | 0.59 | 0.62 | −0.07 | −0.28 | 1.85 | 2.57 | 0.32 | 2.33 | 2.98 |
| SUOX | −0.90 | −0.12 | −0.54 | −0.26 | −0.01 | −0.13 | −0.62 | 0.42 | −0.21 | −1.40 | −0.03 |
| TARS2 | −0.32 | −0.07 | −0.28 | −0.14 | −0.08 | −0.07 | −0.29 | −0.09 | −0.05 | −0.83 | −0.16 |
| TAT | −1.21 | 0.02 | −0.52 | 0.16 | 0.16 | 0.04 | −0.81 | 4.01 | −0.09 | −0.49 | 2.69 |
| TCIRG1 | −0.09 | 0.14 | 0.09 | 0.57 | 0.39 | 0.19 | 0.07 | −0.13 | −0.09 | −0.37 | −1.03 |
| TCTN3 | −0.05 | −0.22 | −0.43 | −0.45 | −0.34 | −0.30 | −0.67 | 0.09 | −0.22 | −1.17 | 0.07 |
| TDO2 | −1.40 | −0.35 | −0.15 | −0.38 | −0.14 | −0.44 | −1.56 | 0.92 | −0.23 | −1.42 | −0.37 |
| TF | −0.34 | −0.25 | −0.17 | −0.07 | −0.09 | −0.17 | −0.46 | 0.29 | −0.19 | −0.03 | 0.18 |
| TFR2 | 0.06 | −0.09 | 0.04 | 0.06 | 0.07 | −0.08 | −0.71 | 0.74 | 0.24 | −0.64 | 0.71 |
| TGFB1 | −0.99 | −0.15 | 0.27 | −0.57 | −0.16 | −0.16 | −1.01 | −1.15 | −0.25 | −2.69 | −0.86 |
| THPO | 0.22 | 0.23 | 0.11 | −0.23 | 0.03 | 0.06 | −0.66 | −0.01 | −0.37 | −2.04 | −1.39 |
| THRB | −1.03 | −0.06 | −0.11 | −0.13 | −0.18 | −0.18 | −0.01 | 0.22 | 0.08 | 0.46 | 0.63 |
| TK2 | −0.29 | −0.09 | 0.00 | −0.20 | 0.15 | 0.06 | −0.89 | −0.07 | 0.41 | −0.66 | −0.89 |
| TMEM135 | −0.06 | −0.16 | 0.19 | −0.25 | −0.41 | −0.27 | 0.30 | 0.69 | 0.01 | 0.49 | 0.81 |
| TMEM165 | −0.19 | −0.15 | −0.24 | −0.21 | −0.17 | −0.13 | −0.46 | −0.15 | −0.05 | −1.22 | −0.56 |
| TMEM67 | 0.11 | 0.14 | 0.08 | −0.29 | 0.04 | −0.01 | −0.84 | −0.69 | 0.24 | −1.17 | −0.09 |
| TMLHE | −0.37 | −0.14 | −0.26 | 0.00 | −0.50 | −0.46 | −0.75 | 0.38 | 0.14 | −0.81 | 0.64 |
| TMPRSS6 | 0.04 | 0.01 | 0.03 | 0.43 | 0.34 | 0.07 | −0.25 | 0.32 | −0.16 | 0.08 | −0.04 |
| TNFRSF1A | −0.42 | −0.06 | 0.04 | −0.17 | −0.13 | −0.12 | −0.50 | −0.30 | −0.14 | −1.11 | 0.41 |
| TNFSF11 | −1.97 | −0.07 | 0.11 | −0.79 | −0.28 | −0.24 | −2.52 | −1.44 | −0.07 | −1.50 | −2.99 |
| TP53 | −0.40 | −0.07 | −0.34 | −0.07 | 0.04 | −0.01 | −0.70 | −0.03 | 0.27 | −0.68 | 0.35 |
| TTC21B | −0.76 | −0.14 | −0.21 | −0.28 | −0.28 | −0.29 | −0.43 | 0.15 | 0.12 | −0.58 | 0.87 |
| TTPA | −1.19 | −0.23 | −0.97 | −0.14 | −0.20 | −0.23 | −1.18 | 0.92 | 0.00 | −0.53 | 0.80 |
| TTR | −0.44 | −0.31 | −0.18 | −0.03 | −0.23 | −0.32 | −0.25 | 0.14 | −0.59 | −0.01 | −0.36 |
| TYMP | −0.08 | −0.09 | 0.12 | 0.38 | 0.37 | 0.27 | 0.60 | −0.20 | −0.65 | 0.33 | −1.81 |
| UBR1 | −0.04 | 0.00 | −0.15 | −0.11 | −0.15 | −0.01 | −0.70 | 0.20 | 0.51 | −1.04 | 0.80 |
| UGT1A1 | 0.08 | −0.35 | −0.68 | −0.42 | −0.38 | −0.41 | −1.71 | 1.35 | 0.80 | −1.38 | 0.30 |
| UGT2B28 | −0.44 | 0.25 | −0.14 | 0.52 | 0.41 | 0.45 | −1.21 | 0.05 | −0.71 | −1.78 | 0.19 |
| UNC13D | 0.15 | 0.25 | 0.24 | 0.71 | 0.44 | 0.38 | 0.47 | −0.13 | −0.07 | −0.13 | −0.71 |
| UPB1 | −0.12 | 0.09 | −0.25 | 0.12 | 0.19 | −0.04 | 0.40 | 0.69 | −0.04 | −0.41 | 1.31 |
| UQCRB | −0.34 | −0.41 | −0.29 | −0.19 | −0.18 | −0.17 | −0.09 | −0.18 | −0.83 | −0.05 | −0.64 |
| UROC1 | −0.74 | −0.47 | −0.37 | −0.23 | −0.26 | −0.17 | −0.76 | 0.31 | −0.02 | −0.70 | −0.03 |
| UTRN | −0.86 | −0.12 | −0.21 | −0.07 | −0.06 | −0.08 | −0.99 | 0.20 | 1.12 | −0.64 | 1.18 |
| VIPAS39 | −0.03 | −0.05 | −0.07 | −0.02 | −0.08 | −0.03 | −0.32 | 0.01 | 0.11 | −0.60 | 0.74 |
| VLDLR | 0.19 | −0.22 | 0.37 | 0.16 | −0.30 | −0.27 | 0.12 | −0.34 | 0.60 | 0.39 | 1.44 |
| VPS13A | 0.12 | 0.31 | 0.28 | 0.24 | 0.08 | 0.11 | −0.22 | 0.33 | 0.82 | −0.35 | 0.13 |
| VPS33B | 0.26 | 0.08 | −0.15 | 0.03 | 0.13 | 0.11 | −0.31 | −0.06 | 0.04 | −1.01 | 0.07 |
| WAS | 0.12 | −0.30 | −0.36 | 0.16 | 0.03 | −0.29 | −0.57 | 0.87 | −0.05 | −0.24 | 0.11 |
| WDR35 | 0.05 | 0.20 | −0.28 | −0.29 | −0.03 | −0.06 | −0.71 | 0.08 | 0.35 | −1.06 | −0.02 |
| XDH | 0.42 | −0.22 | 0.39 | 0.12 | 0.17 | −0.08 | 0.05 | 1.10 | 0.58 | 1.31 | 1.49 |
| YARS2 | −0.30 | −0.25 | −0.38 | −0.40 | −0.36 | −0.30 | −0.66 | −0.53 | −0.30 | −0.90 | −0.40 |
| Gene | 54 | 55 | 61 | 62 | 65 | 66 | 70 | 76 | 79 | 83 | 84 |
| A1BG | −0.89 | 0.01 | 0.12 | 0.21 | 0.33 | 0.48 | 0.84 | 0.06 | 0.51 | −0.47 | −0.1 |
| AARS2 | 0.01 | −0.01 | −0.65 | −0.36 | 0.25 | −0.15 | −1.37 | 0.14 | −0.21 | −0.11 | 0.13 |
| AASS | 0.26 | −0.07 | −0.86 | −0.15 | −0.5 | −0.93 | −0.6 | −0.14 | −0.59 | 0.06 | −0.77 |
| ABAT | 0.38 | −0.12 | −1.25 | −0.5 | −0.44 | −1.12 | −0.97 | −0.22 | −1.07 | 0.02 | −1.42 |
| ABCA1 | 0.94 | 0.05 | −0.23 | −0.41 | −0.59 | −0.97 | 0.25 | −0.12 | −0.36 | −0.08 | −0.43 |
| ABCB11 | 0.89 | 0.13 | −0.32 | −0.25 | 0 | 0.3 | −0.27 | 0.21 | −0.77 | 0.13 | −0.76 |
| ABCB4 | 0.65 | 0.09 | 0.41 | −0.09 | −0.11 | 0.16 | −0.12 | −0.35 | −0.22 | 0.19 | −0.43 |
| ABCC2 | 0.67 | 0.56 | −0.58 | −0.1 | 0.25 | 0.2 | −0.54 | 0.15 | −0.51 | −0.03 | −1.34 |
| ABCC3 | 0.40 | 0.03 | 0.14 | −0.29 | 0.09 | −0.03 | −0.57 | −0.31 | −0.06 | −0.05 | −0.96 |
| ABCC6 | 0.29 | 0.12 | 0.26 | −0.04 | 0.02 | 0.49 | −0.79 | −0.48 | −0.16 | −0.01 | −1.45 |
| ABCD1 | 0.12 | −0.10 | −0.74 | −0.11 | 0.12 | −0.13 | 1.15 | 0.23 | −0.21 | −0.48 | 0.08 |
| ABCD3 | 0.09 | 0.07 | −0.39 | −0.44 | −0.41 | −0.56 | −0.42 | −0.04 | −0.12 | 0.04 | −0.4 |
| ABCG5 | 0.13 | 0.15 | 0.27 | −0.24 | 0.06 | 0.56 | −0.59 | −0.29 | −0.03 | 0.19 | −0.11 |
| ABCG8 | 0.24 | 0.29 | −0.37 | −0.4 | −0.15 | −0.21 | 0.36 | −0.07 | −0.46 | 0.04 | −0.11 |
| ABHD5 | 0.05 | 0.05 | 1.08 | 0.5 | 0.26 | 1.41 | 0.4 | 0.14 | 1.56 | 0.12 | 0.25 |
| ACACA | 0.70 | 0.03 | −0.1 | 0.24 | −0.38 | −0.5 | −1.13 | −0.23 | −0.2 | −0.17 | −2.09 |
| ACACB | 0.57 | 0.17 | 0.28 | 0.49 | −0.1 | 0.31 | −1.47 | −0.42 | −0.36 | 0.06 | −1.86 |
| ACAD9 | 0.01 | −0.03 | −0.5 | −0.16 | 0.07 | −0.27 | −0.35 | 0.07 | −0.09 | −0.13 | −0.83 |
| ACADSB | 0.26 | 0.07 | −0.71 | −0.14 | −0.31 | −0.21 | 0.39 | 0.14 | −0.23 | 0.05 | −0.04 |
| ACADVL | −0.33 | −0.09 | −0.35 | −0.15 | 0.14 | 0.41 | 0.31 | −0.11 | −0.19 | −0.06 | 0.11 |
| ACOT8 | −1.01 | 0.03 | −0.27 | 0.05 | 0.13 | −0.28 | −0.04 | 0.14 | 0.45 | −0.12 | −0.33 |
| ACOX2 | 0.06 | 0.12 | −0.48 | −0.13 | 0.05 | −0.18 | −0.89 | −0.22 | −0.41 | 0.26 | −0.97 |
| ACOX3 | −0.10 | −0.20 | 0.2 | −0.44 | −0.08 | 0.07 | −1.17 | −0.15 | 0.01 | −0.15 | −1.75 |
| ACSF3 | −0.47 | −0.02 | −0.32 | 0 | 0.14 | 0.16 | −1.03 | 0.07 | 0.29 | −0.22 | −0.6 |
| ACSL1 | 0.30 | 0.24 | 0.11 | 0.81 | 0.16 | 0.36 | 0.06 | 0 | 0.11 | 0.03 | −0.12 |
| ACSL3 | 0.09 | 0.02 | 0.44 | 0.44 | 0.01 | −0.18 | 0.95 | 0.27 | 0.15 | −0.06 | 0.16 |
| ACTG2 | 0.15 | 0.28 | −0.53 | −0.31 | −0.72 | −1.5 | 0.31 | −0.17 | −0.43 | −0.07 | −0.13 |
| ACTN1 | 0.44 | −0.21 | 0.03 | −0.36 | −0.05 | −0.29 | −0.44 | −0.29 | −0.28 | −0.21 | −2.21 |
| ADA | −0.29 | −0.03 | −0.24 | −0.48 | −0.02 | −0.7 | 0.46 | 0.2 | −0.36 | −0.16 | −0.72 |
| ADCK3 | −0.21 | 0.12 | −0.36 | −0.45 | 0.05 | 0.17 | 0.18 | 0.15 | −0.2 | −0.11 | 0.18 |
| ADK | −0.52 | −0.07 | −0.45 | −0.18 | −0.18 | −0.5 | 0.41 | −0.07 | 0.45 | 0.04 | −1.7 |
| ADSL | −0.41 | −0.01 | −0.49 | −0.27 | −0.09 | −0.53 | −0.79 | 0.19 | 0.22 | −0.05 | −0.96 |
| AFP | −0.98 | −0.28 | −0.77 | −0.31 | −0.14 | −0.98 | −1.03 | 0.43 | −0.12 | −0.23 | −0.16 |
| AGK | 0.17 | 0.06 | −0.47 | −0.23 | 0.07 | −0.36 | −1.12 | 0.11 | 0.06 | 0.03 | −0.43 |
| AGL | 0.36 | −0.05 | −0.67 | −0.29 | −0.47 | −0.46 | −0.48 | 0 | −0.27 | −0.05 | −0.97 |
| AGPAT2 | −0.97 | −0.09 | 0.04 | 0.53 | 0.39 | 0.28 | 1.3 | 0.13 | 0.45 | −0.59 | −1.09 |
| AGPS | 0.43 | −0.04 | −0.15 | −0.33 | −0.45 | −0.42 | −0.83 | 0.07 | 0.1 | −0.06 | −1.61 |
| AGT | −0.44 | −0.05 | −0.13 | 0 | 0.06 | 0.08 | 0.72 | 0.09 | 0.32 | 0.03 | 0.27 |
| AGTR1 | −0.13 | −0.08 | 0.32 | 0.01 | −0.28 | −0.38 | −0.58 | −0.2 | 0.18 | 0.18 | −0.07 |
| AGXT | −0.66 | 0.04 | −0.55 | −0.24 | 0.1 | −0.04 | 0.21 | 0.02 | 0.2 | −0.28 | −0.66 |
| AHI1 | 0.36 | 0.12 | 0.17 | −0.33 | −0.3 | 0 | −0.81 | −0.14 | −0.01 | 0.3 | −0.61 |
| AHSG | −0.42 | 0.06 | −0.27 | −0.06 | −0.06 | 0.43 | 0.17 | −0.28 | 0.53 | 0.29 | −0.32 |
| AK1 | −0.92 | −0.04 | −0.43 | −0.09 | 0.1 | −0.48 | 0.96 | 0.31 | 0.37 | −0.09 | 0.62 |
| AKR1C1 | −0.83 | 0.02 | −0.13 | 0.06 | 0.15 | 0.17 | −0.52 | 0.49 | 0.35 | −0.07 | −0.96 |
| AKR1C2 | −0.41 | 0.04 | −0.33 | 0.09 | 0.44 | 0.25 | −0.75 | 0.51 | 0.13 | −0.06 | −1.2 |
| AKR1C4 | −0.38 | 0.38 | −0.39 | −0.68 | −0.21 | 0.04 | −1.13 | −0.11 | 0.24 | 0.34 | −1.68 |
| AKR1D1 | −0.10 | 0.35 | 0.07 | 1.24 | 0.48 | 1.48 | 0.5 | −0.03 | 1.16 | 0.39 | 0.17 |
| ALAD | 0.28 | 0.05 | −0.5 | −0.27 | −0.03 | 0.58 | −1.12 | −0.24 | −0.66 | 0.12 | −0.69 |
| ALAS1 | 0.04 | 0.68 | 0.02 | −0.11 | 0.29 | −0.37 | −1.01 | 0.01 | −0.04 | 0.02 | 0.11 |
| ALDH1B1 | −0.06 | −0.07 | −0.02 | −0.23 | 0.13 | −0.07 | −1.28 | −0.08 | −0.06 | −0.08 | −0.86 |
| ALDH2 | −0.60 | 0.05 | 0.06 | 0.04 | 0.09 | 0.55 | 0.62 | 0.08 | 0.54 | −0.08 | −0.04 |
| ALDH6A1 | 0.01 | 0.12 | −0.17 | −0.2 | −0.23 | −0.04 | 0.47 | 0.03 | −0.02 | 0.05 | −0.08 |
| ALDOA | −0.54 | −0.12 | −0.71 | 0.11 | 0.13 | −0.29 | −0.1 | 0.23 | −0.37 | −0.16 | −0.48 |
| ALDOB | 0.00 | 0.05 | 0.48 | 0.08 | 0.02 | 0.89 | −0.23 | −0.1 | 0.18 | 0.27 | −0.27 |
| ALG12 | −0.09 | 0.14 | −0.4 | −0.01 | 0.22 | 0.12 | 0.02 | −0.01 | −0.29 | −0.2 | −0.76 |
| ALMS1 | 0.98 | 0.16 | 0.46 | −0.15 | 0 | 0.29 | −1.13 | −0.05 | −0.54 | 0.06 | −1.35 |
| ALPL | −0.16 | 0.27 | 0.48 | 0.58 | 0.61 | 1.5 | −0.4 | −0.27 | 0.14 | −0.18 | −1.05 |
| AMACR | −0.02 | −0.16 | −0.71 | 0.43 | −0.09 | −0.35 | −0.43 | 0.12 | −0.35 | −0.08 | 0.28 |
| AMPD3 | 0.18 | 0.06 | −0.1 | 0 | 0.35 | 0.54 | 1.1 | 0 | −0.23 | −0.16 | 0.57 |
| AMT | −0.52 | 0.13 | −0.5 | 0.1 | 0.08 | −0.38 | 0.39 | 0.04 | 0.03 | 0 | −0.61 |
| ANGPTL3 | −0.15 | −0.18 | −0.79 | −0.65 | −0.77 | −0.71 | −0.73 | 0.15 | 0.13 | 0.19 | −0.73 |
| ANKS4B | 0.10 | 0.10 | −0.52 | 0.32 | 0.08 | 0.11 | −2.67 | 0.04 | −0.24 | 0.02 | −0.39 |
| ANKS6 | 0.13 | 0.05 | −0.18 | 0.06 | 0.28 | 0.08 | −0.66 | 0.05 | −0.01 | −0.09 | −0.13 |
| ANPEP | 0.18 | 0.05 | −0.61 | 0.11 | 0.1 | 0.05 | −0.09 | −0.11 | −0.4 | 0.09 | −0.47 |
| AOC2 | 0.62 | 0.00 | 0.04 | −0.18 | −0.23 | 0.09 | 0.27 | 0.03 | 0.15 | −0.05 | 0.32 |
| AP3B1 | 0.23 | 0.00 | 0 | −0.09 | −0.15 | −0.34 | −0.65 | −0.12 | −0.13 | 0.15 | −1.36 |
| APC | 0.84 | −0.06 | −0.2 | −0.1 | −0.34 | −0.25 | −0.37 | 0.05 | −0.51 | −0.02 | −0.04 |
| APOA1 | −1.33 | −0.08 | −0.06 | 0.09 | 0.18 | 0.25 | 0.86 | 0.1 | 0.63 | −0.34 | −0.05 |
| APOA2 | −1.19 | −0.02 | −0.44 | 0.18 | 0.12 | −0.2 | 0.61 | 0.16 | 0.46 | 0.11 | 0.31 |
| APOA5 | −0.90 | −0.09 | −0.4 | 0.14 | 0.32 | 0.32 | 0.86 | 0.11 | 0.43 | −0.4 | −0.18 |
| APOB | 1.25 | 0.30 | 0.02 | 0.23 | −0.21 | 0.65 | −0.24 | −0.45 | −0.64 | 0.08 | −0.78 |
| APOC2 | −0.91 | 0.05 | −0.4 | 0 | 0.03 | −0.37 | 0.37 | 0.08 | 0.4 | 0.21 | 0 |
| APOC3 | −0.89 | 0.04 | −0.16 | 0.08 | 0.22 | 0.08 | 0.7 | 0.22 | 0.55 | 0.11 | 0.42 |
| APOE | −1.21 | 0.01 | −0.1 | 0.17 | 0.41 | 0.46 | 0.72 | 0.05 | 0.65 | −0.89 | −0.56 |
| APRT | −1.19 | −0.06 | −0.39 | −0.25 | 0.08 | −0.28 | 0.04 | 0.17 | 0.45 | −0.35 | −0.42 |
| AQP7 | 0.49 | 0.03 | −0.07 | 0.37 | 0.29 | 2.12 | 0.36 | −0.53 | −0.42 | −0.02 | −0.35 |
| AQP9 | 0.09 | 0.11 | 0.01 | 0.74 | 0.42 | 0.19 | 0.33 | −0.04 | −0.2 | 0.09 | −0.05 |
| ARG1 | −0.13 | 0.09 | −0.03 | −0.47 | −0.29 | 0.35 | −0.28 | −0.28 | −1.05 | 0.45 | −1.7 |
| ARG2 | −0.10 | −0.41 | −0.15 | 0.39 | −0.14 | 0.49 | 3.36 | 0.15 | 0.7 | −0.31 | 0.62 |
| ARL13B | −0.37 | 0.00 | 0.14 | −0.14 | −0.17 | −0.34 | −0.68 | 0.18 | 0.31 | 0.2 | −0.67 |
| ARSA | −0.40 | −0.08 | −0.14 | 0.27 | 0.34 | 0.24 | 1.26 | −0.18 | −0.02 | −0.38 | 0.27 |
| ARSB | 0.35 | 0.07 | −0.34 | 0.08 | 0.21 | −0.14 | 0.4 | −0.3 | −0.15 | −0.25 | −0.25 |
| ASL | −0.66 | −0.06 | −0.3 | 0.24 | 0.23 | 0 | −0.25 | 0 | 0.32 | −0.16 | −0.24 |
| ASNS | 0.04 | 0.22 | 1.81 | 0.96 | 0.89 | 2.23 | −0.25 | 0.29 | 3.2 | 0.28 | 1.61 |
| ASPA | 0.72 | −0.33 | 0.63 | −0.99 | −0.08 | 1.18 | 0.81 | −0.05 | −0.01 | 0.14 | 1.55 |
| ASXL1 | 0.28 | −0.09 | −0.08 | −0.33 | −0.13 | 0.09 | −0.58 | −0.11 | −0.31 | −0.1 | −1.04 |
| ATG7 | −0.25 | 0.10 | −0.04 | −0.25 | −0.05 | −0.16 | −0.26 | −0.12 | 0.35 | −0.02 | −1.54 |
| ATIC | −0.16 | 0.01 | −0.46 | −0.48 | −0.13 | −0.69 | −1.49 | 0.08 | 0.09 | 0.06 | −0.74 |
| ATM | 0.91 | 0.09 | 0.3 | −0.16 | −0.21 | −0.07 | −1.12 | −0.19 | −0.28 | 0.1 | −0.5 |
| ATP7A | 0.44 | 0.12 | −0.33 | −0.09 | −0.29 | −0.36 | −0.65 | −0.07 | −0.55 | −0.04 | −0.03 |
| ATP7B | 0.48 | 0.04 | −0.58 | −0.11 | −0.06 | −0.19 | −0.23 | −0.05 | −0.43 | −0.07 | −0.8 |
| ATP8B1 | 0.46 | −0.05 | −0.48 | 0.05 | −0.16 | −0.49 | −0.61 | −0.04 | −0.48 | −0.19 | −2.3 |
| B2M | −0.49 | −0.06 | 0.09 | 0.16 | −0.07 | −0.08 | 0.38 | 0.16 | 0.56 | 0.15 | 0.23 |
| B9D2 | −0.43 | 0.10 | −0.56 | −0.55 | 0.29 | −0.29 | 0.04 | −0.2 | 0.16 | −0.49 | −0.83 |
| BAAT | 0.17 | 0.02 | −0.58 | 0.61 | 0.01 | 0.22 | −1.32 | 0.12 | −0.52 | 0.13 | −1.93 |
| BBOX1 | −0.45 | −0.39 | −0.56 | −0.14 | 0.01 | −0.45 | −1.19 | 0.35 | −0.06 | 0 | −0.77 |
| BBS10 | 0.26 | −0.12 | −0.21 | −0.33 | −0.41 | −0.28 | −0.06 | 0.24 | 0.05 | 0.02 | 0.59 |
| BCHE | 0.02 | −0.07 | −0.63 | −0.41 | −0.5 | −0.79 | −0.69 | −0.07 | 0.12 | 0.13 | −0.67 |
| BCKDHB | −0.16 | 0.07 | −0.78 | −0.31 | −0.06 | −0.38 | 0 | −0.17 | 0.04 | 0.05 | −1.21 |
| BCKDK | −0.21 | 0.00 | −0.48 | −0.23 | 0.02 | −0.45 | −0.91 | 0.04 | −0.23 | −0.37 | −0.42 |
| BCO1 | −0.38 | −0.09 | −1.07 | 0.24 | 0.08 | −0.53 | −0.28 | −0.28 | −0.13 | −0.08 | 0.72 |
| BCR | 0.14 | −0.04 | −0.62 | −0.06 | 0.04 | −0.26 | −0.83 | −0.01 | −0.17 | −0.3 | −1.54 |
| BLNK | 0.09 | −0.03 | 0.53 | 0.39 | 0.18 | 0.75 | 1.32 | −0.2 | −0.18 | 0.06 | 1.31 |
| BLOC1S3 | 0.15 | −0.02 | 0.3 | 0.46 | 0.46 | 0.76 | −0.26 | 0.04 | 0.32 | −0.06 | 0.01 |
| BMPR1A | 0.51 | 0.01 | 0.11 | 0.09 | 0 | 0.07 | −0.33 | −0.05 | −0.06 | −0.02 | −0.9 |
| BRAF | 0.82 | 0.30 | 0.46 | 0.04 | 0.2 | 0.32 | −0.34 | −0.01 | 0.03 | 0.14 | −0.44 |
| BSCL2 | −3.20 | 0.05 | −0.26 | −0.54 | −0.45 | −0.58 | 0.84 | −0.03 | 1.5 | 0.24 | 0.07 |
| BTD | 0.19 | −0.02 | −0.16 | −0.08 | −0.16 | 0.25 | −0.48 | −0.16 | −0.25 | 0.08 | −0.53 |
| C10orf10 | −0.49 | 0.01 | 0.14 | −0.31 | 0.02 | 0.81 | 1.26 | −0.2 | −0.02 | −0.01 | 0.23 |
| C10orf11 | −0.62 | −0.27 | −0.32 | 0.1 | 0.18 | 0.1 | −1.44 | −0.71 | 0.56 | 0.14 | −2.08 |
| C15orf41 | 0.50 | −0.28 | −0.17 | −0.2 | 0.13 | 0.03 | −1.28 | 0.08 | 0.12 | −0.45 | −1.68 |
| C4A | 0.20 | 0.16 | 0.16 | 0.24 | 0.05 | 0.16 | 0.4 | −0.36 | −0.38 | −0.2 | 0.29 |
| C9 | −0.13 | −0.11 | −0.38 | −0.11 | −0.2 | −0.21 | 1.1 | 0.16 | 0.21 | 0.15 | 0.55 |
| CA12 | 0.06 | 0.80 | 0.43 | 0.58 | 0.77 | 0.59 | 0.43 | 0.32 | 0.2 | 0.02 | 0.58 |
| CA2 | −0.44 | 0.59 | 0.76 | 0.6 | 0.53 | 0.48 | 1.36 | 0.11 | 0.71 | 0.15 | 0.9 |
| CA5A | 0.23 | −0.22 | −0.56 | 0.1 | 0.24 | −0.02 | −0.64 | −0.1 | 0.32 | 0.04 | −1.34 |
| CAV1 | 0.15 | −0.21 | −0.02 | −0.81 | −0.47 | −0.47 | 0.02 | 0.13 | 0.34 | 0.02 | −0.74 |
| CCND1 | 0.25 | −0.14 | −0.89 | −0.9 | −0.33 | −2.24 | 0.26 | 0.16 | −0.7 | −0.25 | −0.78 |
| CD320 | −0.95 | −0.04 | −0.95 | −0.04 | 0.17 | −0.58 | −0.9 | 0.17 | 0.3 | −0.29 | −0.96 |
| CD46 | 0.05 | −0.05 | 0.34 | 0.24 | −0.14 | −0.02 | 1.34 | 0.08 | 0.23 | 0.15 | 0.43 |
| CD81 | −0.76 | −0.02 | −0.26 | 0.27 | 0.3 | 0.02 | 0.46 | 0.09 | 0.17 | −0.11 | 0.03 |
| CDAN1 | 0.10 | −0.07 | −0.32 | −0.04 | 0.25 | 0.02 | −0.23 | 0.11 | −0.27 | −0.08 | 0.38 |
| CDH1 | 0.50 | 0.01 | −0.29 | −0.12 | −0.29 | −0.25 | −0.12 | −0.56 | −0.59 | 0.06 | −1.79 |
| CEP164 | 0.05 | −0.01 | −0.48 | −0.12 | 0.12 | −0.32 | 1.34 | −0.03 | −0.21 | −0.1 | 0.23 |
| CEP19 | −0.43 | −0.22 | −0.54 | −0.06 | −0.07 | −0.43 | 0.44 | −0.02 | 0.1 | −0.07 | −0.28 |
| CEP290 | 0.01 | 0.06 | 0.41 | −0.26 | −0.14 | −0.17 | −1.26 | 0.16 | 0.55 | 0.44 | 0.21 |
| CEP83 | 0.33 | −0.04 | 0.05 | −0.3 | −0.12 | −0.13 | −1.24 | −0.06 | −0.15 | 0.11 | −0.39 |
| CFB | −0.28 | −0.07 | 0.26 | 0.1 | 0.02 | 0.17 | 0.72 | 0.01 | 0.23 | 0.1 | 0.29 |
| CFHR2 | −0.93 | 0.01 | −0.22 | −0.08 | −0.46 | −0.16 | 1.2 | 0.13 | 0.4 | 0.35 | 0.7 |
| CHRM3 | 0.20 | 0.08 | 0.06 | 0.28 | −0.06 | −0.14 | −0.08 | −0.58 | −0.27 | −0.17 | −2.02 |
| CIDEC | −0.40 | 0.28 | −1.38 | −0.13 | 0.1 | 0.24 | −0.39 | −0.06 | −0.79 | 0.08 | −0.6 |
| CLDN1 | 0.16 | −0.05 | −0.26 | −0.46 | −0.37 | −0.71 | −0.93 | −0.05 | 0.09 | −0.01 | −0.83 |
| COG1 | 0.25 | 0.17 | −0.08 | −0.19 | 0.06 | −0.28 | 0.38 | 0.12 | −0.37 | −0.19 | 0.5 |
| COG5 | 0.35 | −0.06 | 0.11 | −0.14 | −0.22 | −0.03 | −0.45 | −0.12 | 0.24 | −0.02 | −1.94 |
| COG6 | 0.23 | −0.01 | −0.09 | 0.1 | −0.08 | 0.08 | −0.46 | 0.18 | 0.29 | −0.09 | −0.28 |
| COL1A1 | 0.34 | −0.34 | 0.18 | 0.41 | 0.09 | 0.48 | −0.72 | −0.09 | −0.43 | −0.35 | 0.45 |
| COX6A1 | −1.30 | −0.13 | −0.08 | 0.02 | −0.01 | −0.07 | 0.16 | 0.29 | 0.62 | −0.05 | 0.27 |
| CP | 0.80 | 0.11 | 0.77 | 0.26 | −0.27 | 0.16 | 0.53 | −0.16 | −0.27 | 0.36 | 0.19 |
| CPS1 | 0.69 | 0.09 | −0.89 | 0.52 | −0.63 | 0.84 | −0.28 | −0.56 | −0.75 | 0.2 | −0.79 |
| CPT1A | −0.10 | −0.18 | 0.11 | 0.19 | 0.13 | 1.06 | 0.16 | −0.03 | −0.4 | 0.08 | −1.18 |
| CPT2 | −0.15 | 0.10 | −0.16 | −0.2 | 0.05 | 0.33 | 1.26 | 0.28 | −0.1 | 0.07 | 1.3 |
| CROT | −0.21 | −0.04 | −0.49 | −0.4 | −0.4 | −0.69 | 0.55 | 0.19 | −0.33 | −0.02 | 0.23 |
| CSPP1 | −0.22 | −0.07 | 0.24 | −0.14 | −0.05 | 0.17 | −0.7 | −0.09 | 0.17 | 0.35 | −0.45 |
| CTC1 | 0.46 | −0.08 | −0.01 | 0.03 | 0.2 | 0.2 | −0.9 | 0.04 | −0.2 | −0.04 | 0.65 |
| CTDSP2 | 0.44 | 0.10 | 0.52 | −0.14 | −0.07 | 0.56 | −0.18 | −0.06 | 0.03 | 0 | −0.36 |
| CTH | −0.17 | 0.23 | 1.17 | 0.67 | 0.6 | 1.87 | 0.66 | 0.15 | 2.52 | 0.11 | 1.32 |
| CTNS | 0.16 | 0.08 | −0.11 | 0.69 | 0.65 | 0.42 | −1.18 | 0.1 | −0.12 | −0.16 | −0.39 |
| CTSF | −0.63 | 0.10 | 0.25 | 0.19 | 0.3 | 0.68 | 0.6 | 0.07 | 0.3 | −0.1 | 0.01 |
| CTSK | 0.47 | −0.15 | 0.31 | 0.08 | 0.04 | −0.49 | 0.02 | −0.13 | 0 | 0.32 | 1.01 |
| CYB5A | −0.56 | 0.73 | −0.12 | −0.09 | 0.34 | 0.43 | −0.16 | −0.01 | 0.21 | 0.09 | −0.54 |
| CYB5R3 | 0.02 | 0.04 | −0.21 | 0.1 | 0.13 | 0.05 | 0.14 | 0.05 | 0.07 | −0.25 | −0.57 |
| CYP27A1 | −0.43 | 0.04 | −0.18 | 0.14 | 0.28 | 0.51 | 0.51 | −0.03 | 0.12 | 0.02 | −0.12 |
| CYP2B6 | 0.34 | 0.48 | 0.25 | −0.08 | 0.12 | 0.58 | 0.41 | −0.04 | 0.09 | 0.24 | 0.2 |
| CYP2C19 | 1.62 | 0.24 | 0.54 | 0.1 | −0.31 | 0.2 | −1 | −0.79 | −0.14 | 0.27 | −1.4 |
| CYP2D6 | −0.62 | 0.11 | −0.06 | 0.16 | 0.22 | 0.47 | 0.79 | −0.01 | 0.24 | −0.3 | 0.27 |
| CYP39A1 | −0.08 | 0.43 | −0.07 | 0.08 | −0.11 | 0.62 | −0.24 | −0.23 | 0 | 0.06 | −0.62 |
| CYP3A4 | 0.01 | 3.12 | 0.07 | 0.11 | 0.77 | 2.11 | 1.58 | 0.18 | 0.49 | 0.18 | 0.96 |
| CYP7B1 | 0.26 | 0.05 | −0.46 | −0.3 | −0.34 | 0.28 | −0.02 | −0.27 | −0.44 | −0.17 | −1.05 |
| DAO | −0.01 | −0.04 | −1.06 | −0.11 | 0.13 | −1.2 | 0.23 | −0.06 | −0.72 | 0.02 | −0.79 |
| DBT | 0.28 | 0.04 | −0.13 | −0.16 | −0.32 | −0.43 | −0.33 | −0.01 | 0 | 0.18 | −0.27 |
| DCDC2 | 0.20 | 0.01 | −1.43 | −1.04 | −0.6 | −2.5 | −1.25 | −0.57 | −0.51 | 0.25 | −2.14 |
| DCXR | −1.16 | −0.01 | −0.74 | 0.01 | 0.16 | −0.23 | 0.96 | 0.23 | 0.37 | −0.35 | −0.21 |
| DDC | −0.51 | 0.11 | −1.21 | −0.6 | −0.25 | −0.66 | −0.43 | 0.14 | 0.14 | −0.04 | −0.74 |
| DDO | 0.06 | −0.03 | −0.47 | 0.15 | 0.12 | −0.59 | −0.11 | −0.21 | −0.06 | 0.21 | 0.12 |
| DENR | −0.42 | −0.05 | −0.26 | −0.08 | −0.13 | −0.46 | −0.22 | 0.13 | 0.35 | 0.07 | −0.2 |
| DHCR24 | 0.26 | 0.12 | −0.12 | 0.17 | −0.14 | −0.22 | −0.36 | −0.04 | −0.25 | −0.09 | −1.07 |
| DHCR7 | 0.15 | −0.01 | 0.08 | 1.4 | 0.32 | −0.42 | −0.23 | 0.02 | −0.18 | −0.11 | −0.04 |
| DHODH | −0.39 | 0.00 | −0.3 | −0.14 | 0.22 | −0.12 | −1.41 | −0.06 | 0.16 | 0.1 | −0.64 |
| DIS3L2 | 0.23 | −0.11 | −0.39 | −0.37 | −0.14 | −0.44 | −0.92 | −0.36 | −0.23 | −0.05 | −2.21 |
| DKC1 | −0.28 | −0.03 | −0.37 | −0.23 | −0.05 | −0.42 | −1.33 | 0.26 | 0.58 | 0.05 | −0.34 |
| DLAT | 0.02 | −0.09 | 0.01 | 0.16 | −0.21 | −0.77 | −1.1 | 0.07 | 0.04 | −0.1 | −0.67 |
| DLD | −0.10 | 0.00 | −0.33 | −0.21 | −0.3 | −0.52 | −0.51 | 0.08 | 0.04 | 0.07 | −0.63 |
| DMGDH | 0.17 | 0.15 | −0.21 | −0.14 | −0.15 | −0.16 | 0.24 | 0.2 | −0.02 | 0.16 | −0.14 |
| DNAH5 | 0.70 | −0.03 | 0.66 | −0.29 | −0.1 | 0.34 | −1 | −0.23 | −0.39 | 0.05 | −1.27 |
| DNM1L | 0.30 | −0.08 | −0.19 | −0.13 | −0.12 | −0.41 | −0.49 | 0.22 | −0.06 | 0 | −0.51 |
| DOLK | −0.21 | −0.04 | −0.2 | 0.09 | 0.33 | −0.26 | 0.19 | 0.1 | −0.06 | −0.16 | 0.71 |
| DPM3 | −1.37 | −0.06 | −0.1 | 0.29 | 0.68 | 0.19 | 0.16 | 0.24 | 0.52 | −0.42 | −0.19 |
| DPYD | 0.34 | 0.17 | −0.24 | −0.11 | −0.43 | −0.18 | −0.61 | −0.96 | 0 | 0.02 | −2.58 |
| DPYS | 0.12 | 0.23 | −0.95 | −0.39 | −0.27 | −0.23 | −0.34 | −0.13 | −0.21 | 0.06 | −1.22 |
| EARS2 | 0.29 | 0.09 | −0.5 | −0.29 | −0.01 | −0.42 | −0.63 | 0.03 | −0.26 | −0.08 | −0.77 |
| EDA | 0.29 | −0.02 | 0.1 | 0.61 | 0.3 | 0.48 | 0.91 | −0.77 | −0.33 | −0.18 | −1.29 |
| EHHADH | 0.23 | 0.05 | −0.49 | −0.25 | −0.3 | −0.31 | −0.33 | −0.09 | −0.43 | 0.17 | −0.33 |
| ELAC2 | −0.14 | 0.02 | −0.53 | −0.35 | −0.13 | −0.4 | −0.76 | 0.21 | −0.08 | −0.17 | −0.41 |
| ENO3 | −0.48 | −0.27 | −0.1 | 0.02 | −0.12 | −0.85 | 1.32 | 0.05 | −0.16 | −0.01 | −0.09 |
| ENPP1 | 0.67 | 0.01 | 0.15 | 0.02 | −0.21 | 0.17 | −0.45 | −0.1 | 0.16 | 0.18 | −0.49 |
| EPG5 | 0.72 | 0.20 | 0.27 | 0.24 | 0 | 0.24 | −0.41 | −0.12 | −0.43 | −0.03 | −0.69 |
| EPHX1 | −0.33 | 0.35 | −0.14 | −0.26 | 0.16 | 0.26 | 0.78 | 0.12 | 0 | 0.04 | 0.08 |
| ERCC4 | 0.36 | −0.05 | −0.13 | −0.05 | −0.18 | −0.2 | −0.31 | −0.02 | 0.01 | 0.02 | 0.4 |
| ERCC6 | 0.61 | 0.16 | 0.24 | −0.2 | −0.09 | 0.08 | 0.09 | 0.04 | −0.12 | 0 | 0.03 |
| ETFB | −1.19 | −0.07 | −0.38 | −0.02 | 0.14 | 0.03 | −0.1 | 0.14 | 0.42 | −0.11 | −0.55 |
| ETHE1 | −0.57 | −0.15 | −0.03 | 0.02 | 0.31 | 0.18 | 0.03 | 0.02 | 0.42 | −0.13 | −0.36 |
| F2 | −0.40 | −0.03 | 0.13 | 0.27 | 0.31 | 0.29 | 0.3 | −0.03 | 0.24 | −0.05 | −0.09 |
| F2RL1 | −0.05 | 0.01 | −0.14 | 0.35 | 0.01 | 0.29 | 0.55 | 0.2 | 0.24 | −0.11 | 1.17 |
| F5 | 0.68 | 0.09 | −0.04 | −0.35 | −0.44 | 0.35 | 0.21 | −0.24 | −0.52 | 0.12 | −0.05 |
| F9 | 0.08 | 0.05 | −0.14 | −0.12 | −0.17 | −0.25 | 0.92 | 0.16 | 0.17 | 0.04 | 0.29 |
| FABP1 | −0.93 | −0.25 | 0.07 | 0.06 | −0.05 | −0.17 | 0.4 | −0.33 | 0.58 | 0.25 | −0.95 |
| FADD | −0.53 | −0.08 | −0.45 | −0.08 | 0.13 | −0.29 | −0.23 | 0.17 | 0.43 | −0.32 | −0.41 |
| FAM111A | 0.49 | 0.07 | 0.1 | 0.12 | −0.05 | −0.05 | −0.68 | −0.14 | −0.26 | 0.18 | 0.93 |
| FAM111B | 0.47 | 0.03 | −2.15 | −1.27 | −0.44 | −2.02 | −0.13 | 1.04 | −1.74 | 0.01 | −1.24 |
| FAN1 | 0.43 | 0.05 | −0.03 | −0.37 | −0.14 | −0.36 | −1.14 | 0.11 | 0.14 | 0.06 | 0.32 |
| FARS2 | −0.55 | −0.14 | −0.42 | −0.21 | −0.04 | −0.34 | −0.84 | −0.7 | 0.06 | −0.06 | −3 |
| FASLG | 0.35 | −0.21 | −1.32 | −0.55 | −0.66 | −1.44 | −1.48 | −0.31 | −1.19 | −0.16 | −1.15 |
| FGA | −0.10 | 0.16 | −0.32 | 0.07 | −0.37 | 0.12 | 0.87 | −0.24 | −0.25 | 0.15 | 0.46 |
| FGD1 | 0.29 | −0.07 | −0.46 | −0.4 | 0.14 | −0.27 | −0.22 | 0.24 | −0.63 | −0.42 | −1.37 |
| FGFR2 | 0.75 | 0.07 | −0.13 | −0.97 | −0.42 | −0.22 | −0.16 | −1.3 | 0.15 | 0.36 | −2.48 |
| FIG4 | 0.08 | −0.12 | −0.07 | 0.29 | 0.3 | 0.06 | −0.45 | −0.09 | 0 | 0.29 | −0.69 |
| FIS1 | −1.07 | −0.06 | −0.35 | −0.06 | 0.25 | −0.18 | −0.27 | 0.19 | 0.52 | 0.01 | −0.22 |
| FTCD | −0.35 | 0.31 | −0.56 | 0.18 | 0.43 | 0.32 | −0.68 | −0.37 | −0.05 | −0.28 | −1.66 |
| FTH1 | −0.84 | 0.06 | 0.44 | 0.45 | 0.7 | 0.48 | −0.17 | 0.34 | 0.6 | −0.33 | −0.49 |
| FUCA1 | −0.37 | −0.11 | −0.13 | 0.02 | 0.07 | 0.24 | 1.44 | 0.04 | 0.01 | 0.06 | 0.43 |
| FXN | −0.10 | 0.23 | −0.22 | −0.33 | 0.12 | −0.02 | −1.56 | −0.11 | −0.17 | −0.37 | −1.04 |
| G6PC | 0.69 | 0.12 | 0.6 | 1.05 | 0.01 | 3.27 | 0.32 | −0.55 | −0.17 | −0.01 | −0.03 |
| G6PC3 | −0.60 | 0.02 | −0.52 | −0.17 | 0.29 | −0.25 | −0.89 | 0.11 | −0.07 | −0.21 | −0.38 |
| G6PD | −0.23 | 0.13 | −0.41 | 0.7 | 0.59 | 0.13 | 1.51 | 0.55 | −0.05 | −0.09 | 1.7 |
| GALK1 | −0.94 | −0.03 | −0.61 | 0.05 | 0.34 | −0.37 | −0.27 | 0.19 | 0.4 | −0.45 | −0.7 |
| GALK2 | −0.51 | 0.00 | −0.14 | 0.08 | −0.11 | −0.25 | 0.04 | 0.04 | 0.22 | −0.06 | −1.47 |
| GALT | −0.13 | 0.01 | −0.58 | 0.37 | 0.26 | 0.41 | −0.02 | −0.13 | −0.21 | 0 | −0.43 |
| GATA6 | −0.26 | −0.07 | 0 | −0.01 | −0.02 | 0.11 | −0.1 | −0.12 | 0.17 | −0.71 | −0.77 |
| GATM | 0.12 | −0.15 | −0.52 | −0.21 | −0.37 | −0.46 | −0.66 | −0.13 | −0.33 | −0.01 | −1.11 |
| GBA | 0.04 | 0.02 | −0.23 | 0.33 | 0.27 | −0.11 | −0.06 | 0.02 | −0.07 | 0.02 | −0.38 |
| GBE1 | −0.07 | −0.15 | −0.11 | 0.06 | −0.08 | 0.2 | −0.62 | −0.01 | 0.39 | −0.06 | −2.25 |
| GCH1 | −0.12 | −0.06 | −0.19 | 0.02 | −0.25 | −0.38 | 1.49 | 0.04 | 0.23 | −0.12 | 0.43 |
| GCKR | 0.17 | 0.23 | −0.06 | 0.43 | 0.32 | −0.08 | −1.15 | −0.15 | −0.19 | 0.14 | −0.16 |
| GCLC | 0.11 | −0.04 | −0.02 | 0.07 | −0.14 | 0.14 | 0.3 | 0.18 | 0.13 | −0.06 | −0.18 |
| GOSH | −1.35 | 0.46 | −1.21 | −0.26 | 0.44 | −0.4 | −0.39 | 0.18 | 0.22 | −1.23 | −1.49 |
| GGCX | 0.17 | 0.19 | 0.04 | −0.27 | −0.22 | −0.26 | 0.29 | −0.22 | −0.1 | 0.08 | −0.17 |
| GHR | 0.73 | 0.10 | 0.05 | −0.23 | −0.6 | −0.7 | −0.71 | −0.53 | −0.67 | 0.29 | −2.23 |
| GJB2 | −0.07 | 0.03 | −0.28 | 0 | 0.1 | 0.06 | 0.44 | 0.02 | 0.04 | 0 | −0.9 |
| GLDC | −0.17 | −0.04 | −1.74 | −0.51 | −0.33 | −1.49 | 0.19 | −0.09 | −0.7 | 0.06 | −1.1 |
| GLIS3 | 0.79 | −0.01 | 0.25 | −0.55 | −0.41 | 0.13 | −1.26 | −0.45 | 0.04 | −0.18 | −2.76 |
| GLS2 | −0.27 | −0.02 | −0.47 | −0.25 | −0.06 | −0.58 | 2.8 | 0.34 | 0 | −0.01 | 1.76 |
| GLYCTK | −0.44 | −0.01 | 0.05 | 0.06 | 0.43 | 0.67 | −0.45 | −0.35 | 0.13 | −0.24 | −1.52 |
| GNE | 0.19 | −0.03 | 0.53 | 0.41 | −0.11 | 0.06 | −0.52 | 0.19 | −0.03 | 0.02 | −0.04 |
| GNPTAB | 0.69 | −0.09 | −0.01 | −0.03 | −0.13 | −0.31 | 0.01 | 0.03 | −0.63 | 0.1 | −0.07 |
| GPC3 | −0.26 | 0.38 | 0.47 | 0.12 | 0.06 | 0 | 1.26 | −0.14 | 0.22 | 0.23 | 0.77 |
| GPD1 | −0.27 | −0.19 | −1.54 | −0.2 | 0 | −1.08 | −0.82 | 0.17 | −0.18 | 0.01 | −0.44 |
| GPHN | 0.30 | 0.06 | −0.14 | −0.11 | 0.06 | 0.05 | −0.43 | −0.66 | −0.11 | −0.01 | −2.77 |
| GTPBP3 | 0.06 | −0.07 | −0.65 | −0.14 | 0.07 | 0.04 | −1.15 | 0.01 | 0 | −0.59 | −0.26 |
| GUSB | −0.55 | −0.06 | 0.08 | 0.17 | 0.23 | 0.29 | 0.34 | −0.05 | 0.23 | 0.02 | 0.11 |
| GYG1 | −0.38 | −0.08 | 0.13 | 0 | 0.02 | −0.16 | 0.33 | 0.2 | 0.09 | 0.02 | 0.25 |
| GYS1 | 0.31 | −0.01 | −0.68 | −0.14 | 0.12 | −0.4 | −0.29 | −0.01 | −0.66 | −0.11 | −0.66 |
| GYS2 | 0.10 | −0.21 | −0.25 | 0.32 | −0.13 | 0.73 | 1.46 | −0.28 | −0.25 | −0.09 | 0.02 |
| H19 | −0.22 | 0.25 | −0.34 | −0.07 | 0.34 | −0.21 | 0.79 | 0.25 | 0.3 | −0.33 | −0.17 |
| H6PD | 0.35 | −0.02 | 0.17 | −0.29 | −0.01 | 0.38 | −1.02 | −0.41 | −0.52 | 0 | −1.09 |
| HAL | 0.20 | 0.32 | −0.24 | −0.35 | −0.17 | 0.27 | 1.58 | −0.47 | −0.39 | 0.39 | 0.4 |
| HAX1 | −0.51 | 0.00 | 0.13 | 0.19 | 0.15 | 0.2 | −0.67 | 0.28 | 1.11 | −0.01 | 0.37 |
| HBA1 | −1.50 | 1.05 | 1.89 | −0.09 | 0.04 | 0.84 | −0.27 | −0.51 | −0.14 | −0.75 | −0.49 |
| HBB | −0.64 | 2.16 | 1.45 | 1 | −0.54 | 1.65 | 1.03 | −0.82 | 0.32 | 0.93 | 1.54 |
| HGD | 0.00 | 0.05 | −1.1 | −0.45 | −0.18 | −0.1 | −0.83 | 0.09 | −0.18 | 0.04 | −0.74 |
| HLA-B | −0.68 | −0.08 | −0.1 | 0.06 | 0.18 | 0.02 | 0.5 | −0.03 | 0.42 | −0.19 | 0.03 |
| HMBS | −0.71 | −0.11 | −0.71 | −0.2 | 0 | −0.46 | −1.06 | 0.16 | 0.37 | −0.09 | −0.92 |
| HMGCR | 0.16 | −0.03 | 0.35 | 1.24 | −0.29 | −0.37 | 0.1 | 0.13 | −0.29 | 0.04 | 0.37 |
| HMGCS2 | 0.02 | 0.26 | −1.02 | 0.55 | −0.28 | 3.01 | 0.71 | −0.58 | −1.26 | 0.41 | −1.16 |
| HMOX1 | −0.30 | −0.04 | 0.01 | 0.81 | 1.28 | 0.6 | −0.96 | 0.53 | 0.74 | −0.16 | −1.07 |
| HNF1A | −0.09 | 0.13 | −0.58 | −0.19 | 0.11 | 0.15 | −1.07 | 0 | −0.15 | −0.26 | −0.99 |
| HNF1B | 0.41 | −0.08 | −0.14 | −0.51 | −0.15 | 0.01 | −0.61 | −0.29 | −0.54 | −0.03 | −1.34 |
| HNF4A | 0.31 | −0.05 | −0.02 | 0.14 | −0.01 | 0.34 | −1.28 | −0.16 | −0.45 | −0.04 | −1.65 |
| HOGA1 | 0.00 | 0.27 | −1.24 | −0.26 | 0.01 | −0.56 | −0.39 | −0.49 | −0.48 | −0.03 | −1.87 |
| HPD | −0.36 | 0.01 | −0.32 | −0.23 | 0.06 | −0.42 | 0.13 | 0.14 | −0.14 | 0 | −0.59 |
| HPGD | −0.15 | −0.23 | −0.93 | −0.09 | −0.7 | −0.58 | −0.28 | −0.08 | −0.44 | −0.03 | −0.84 |
| HPS4 | 0.24 | −0.10 | −0.03 | 0.21 | 0.26 | 0.4 | −0.71 | 0.04 | 0.15 | 0.13 | 0.35 |
| HPS5 | 0.32 | −0.06 | −0.53 | 0.06 | −0.18 | 0.33 | 0.13 | 0.14 | −0.04 | −0.01 | 0.11 |
| HPS6 | −0.60 | 0.03 | −0.61 | 0.06 | 0.23 | 0.03 | 0.12 | 0.26 | −0.12 | −0.29 | 0.24 |
| HPX | −0.65 | −0.02 | 0.16 | 0.13 | 0.2 | 0.32 | 0.79 | 0.09 | 0.48 | 0.05 | 0.29 |
| HRAS | −1.09 | −0.18 | −0.1 | 0.36 | 0.31 | 0.17 | 1.72 | 0.3 | 0.58 | −0.17 | 0.67 |
| HRSP12 | −0.87 | −0.06 | −0.39 | −0.24 | −0.3 | −0.57 | 0.6 | 0.31 | 0.54 | 0.09 | 0.1 |
| HSD17B10 | −1.14 | −0.02 | −0.4 | −0.04 | 0.15 | −0.34 | 0.25 | 0.17 | 0.36 | −0.01 | −0.32 |
| HSD3B7 | −0.26 | −0.05 | −1.05 | 0.36 | 0.26 | −0.32 | −0.86 | 0.12 | −0.12 | −0.31 | −0.75 |
| HTT | 0.52 | 0.03 | −0.66 | −0.22 | −0.21 | −0.43 | −0.24 | −0.13 | −0.59 | −0.18 | −1.44 |
| HYAL1 | −0.38 | −0.03 | −0.31 | 0.26 | 0.06 | −0.59 | 0.68 | 0.03 | −0.11 | 0.04 | −0.03 |
| HYLS1 | −0.02 | −0.23 | −0.41 | −0.27 | −0.16 | −0.52 | −0.68 | 0.45 | 0.44 | −0.01 | 0.53 |
| ID1 | −0.99 | −0.12 | −2.21 | −0.49 | −0.69 | −4.39 | −0.52 | −0.37 | −1.8 | −0.19 | −3.64 |
| IDH1 | −0.11 | 0.15 | 0.19 | 0.79 | 0.19 | 0.56 | −0.37 | 0.11 | 0.61 | 0.12 | −0.49 |
| IDH2 | −0.31 | 0.02 | −0.13 | −0.2 | 0 | −0.38 | 0.55 | 0 | −0.2 | −0.04 | −0.68 |
| IDUA | −0.38 | 0.09 | −0.07 | −0.01 | 0.34 | 0.28 | 0.77 | −0.25 | 0.16 | −0.74 | −0.46 |
| IFIH1 | 0.05 | −0.07 | −0.16 | −0.21 | −0.06 | −0.12 | 0.21 | 0.08 | 0.1 | −0.04 | 0.03 |
| IFNAR2 | 0.18 | −0.01 | 0.19 | −0.21 | 0.01 | −0.33 | −1.17 | −0.15 | −0.18 | 0.02 | −1.15 |
| IFT122 | 0.01 | 0.02 | −0.11 | −0.53 | −0.16 | −0.22 | −1.33 | −0.16 | −0.41 | 0.02 | −1.03 |
| IFT140 | 0.40 | −0.06 | 0.17 | −0.16 | 0.27 | 0.53 | −0.1 | −0.35 | −0.19 | 0 | −1.16 |
| IFT172 | 0.43 | 0.11 | 0.03 | −0.09 | 0.16 | 0.28 | −1.65 | −0.55 | −0.48 | 0.12 | −0.84 |
| IFT27 | −0.99 | −0.02 | −0.25 | 0.11 | 0.27 | −0.14 | −0.54 | 0.13 | 0.53 | 0.01 | −0.01 |
| IFT80 | 0.46 | −0.06 | −0.05 | 0.01 | −0.41 | −0.71 | −0.15 | −0.13 | −0.26 | 0.19 | −0.95 |
| IGFALS | −0.36 | −0.02 | −0.78 | −0.2 | 0.11 | −0.36 | −0.05 | −0.04 | −0.37 | −1.04 | −1.57 |
| IKBKG | −0.26 | −0.09 | −0.53 | 0.12 | 0.3 | 0.06 | −0.44 | 0.26 | −0.02 | −0.22 | −1.45 |
| IL1RN | 0.27 | 0.02 | 0.58 | 0.68 | 0.21 | 1.53 | −0.59 | −0.16 | 0.51 | −0.06 | −1.41 |
| IL2RG | 0.38 | −0.04 | −0.41 | 0.45 | 0.6 | 0.14 | −1 | −0.06 | 0.27 | −0.24 | 0.52 |
| INSR | 0.56 | 0.11 | 0.38 | 0.63 | 0.28 | 1.16 | −0.67 | −0.18 | −0.29 | 0.04 | −1.23 |
| INVS | 0.54 | 0.12 | 0.04 | −0.05 | −0.03 | 0.05 | −0.5 | −0.27 | −0.3 | 0.05 | −1.08 |
| ISYNA1 | −0.41 | −0.10 | −0.3 | −0.04 | −0.17 | 0.22 | 2.78 | 0.17 | 0.29 | −0.48 | 0.16 |
| ITGB3 | 0.85 | −0.40 | 0.29 | 0.38 | 0.27 | 0.7 | −0.86 | −0.14 | 0.14 | −0.23 | −1.94 |
| IYD | 0.71 | 0.09 | −0.67 | −0.25 | −0.02 | −0.25 | −0.8 | −0.11 | −0.25 | 0.19 | −0.59 |
| JAG1 | 0.41 | −0.23 | −0.11 | −0.2 | −0.1 | −0.55 | 0.68 | −0.16 | −0.34 | −0.15 | −0.06 |
| JAK2 | 0.54 | −0.11 | 0.23 | 0.24 | −0.14 | 0.09 | −0.15 | 0.1 | −0.13 | 0 | 0.15 |
| KCTD11 | −0.27 | −0.04 | −0.04 | −0.29 | −0.21 | −0.33 | 1.23 | 0.1 | −0.24 | −0.14 | 0.68 |
| KCTD7 | 0.47 | −0.19 | 0.12 | 0.41 | 0.11 | 0.27 | 0.63 | 0.28 | −0.2 | 0.02 | 0.76 |
| KHK | −0.69 | −0.09 | −1.25 | 0.08 | 0.03 | −0.62 | −0.28 | 0.05 | −0.08 | −0.02 | −0.99 |
| KIAA0586 | 0.87 | 0.02 | 0.02 | −0.2 | −0.2 | −0.23 | −0.56 | 0.03 | −0.35 | 0.03 | −0.49 |
| KMT2D | 1.26 | 0.06 | −0.33 | −0.15 | −0.05 | 0.24 | 0.01 | 0.05 | −0.66 | −0.31 | −0.32 |
| KPTN | −0.53 | −0.16 | −0.57 | 0.08 | 0.22 | 0.2 | 0.39 | 0.13 | 0.24 | −0.1 | −0.41 |
| KRIT1 | 0.61 | 0.08 | 0.18 | −0.14 | −0.1 | −0.1 | −0.43 | 0.02 | 0.04 | 0.2 | 0.08 |
| KRT18 | −0.57 | −0.28 | −0.53 | −0.3 | −0.47 | −0.74 | −1.04 | 0 | −0.05 | −0.2 | −0.99 |
| KYNU | −0.39 | −0.03 | 0.09 | 0.32 | 0.16 | −0.31 | 0.07 | 0.46 | 0.28 | 0.1 | −1.33 |
| LAMB2 | 0.00 | −0.07 | −0.57 | −0.04 | 0.01 | 0.07 | −0.6 | −0.11 | −0.4 | −0.19 | −0.54 |
| LCAT | −0.33 | −0.14 | −0.24 | −0.19 | 0.11 | −0.14 | 0.84 | −0.04 | 0.05 | −0.08 | 0.07 |
| LDHB | −0.90 | 0.37 | −0.98 | −0.5 | −0.34 | −1.09 | −0.86 | 0.49 | 0.42 | −0.15 | −0.42 |
| LDLR | 0.29 | 0.02 | 1.03 | 1.31 | 0.24 | 0.44 | −0.07 | −0.34 | 0.24 | −0.04 | −0.84 |
| LDLRAP1 | −0.15 | −0.01 | −0.35 | −0.06 | 0.2 | −0.13 | 0.55 | 0.23 | −0.13 | −0.05 | −0.06 |
| LIPC | −0.18 | −0.02 | −0.76 | −0.59 | −0.29 | −0.61 | −1.61 | −0.44 | −0.11 | −0.03 | −2.13 |
| LIPT1 | −0.54 | 0.20 | 0.36 | −0.12 | −0.12 | −0.06 | −0.01 | 0.26 | 0.49 | 0.34 | 0.9 |
| LMNB2 | −0.04 | −0.08 | −0.64 | −0.32 | 0.14 | −0.54 | −0.77 | 0.04 | −0.24 | −0.48 | −0.87 |
| LPA | −0.23 | −0.06 | 0.41 | 0.17 | 0.07 | −0.08 | 0.63 | −0.17 | 0.17 | 0.14 | 0.01 |
| LPIN1 | 0.21 | 0.07 | 0.23 | 1.25 | 0.22 | 0.08 | −0.24 | 0.01 | −0.11 | 0.09 | −0.35 |
| LPIN2 | 0.43 | 0.10 | 0.35 | 0.3 | 0.1 | 1.34 | −0.58 | −0.12 | 0.04 | 0.2 | −0.23 |
| LRBA | 0.89 | −0.04 | −0.21 | −0.21 | −0.32 | −0.3 | −1.11 | −0.69 | −0.44 | −0.07 | −2.98 |
| LRP5 | 0.28 | 0.03 | −0.54 | −0.14 | 0.15 | −0.03 | −0.53 | −0.26 | −0.35 | −0.39 | −1.72 |
| LYRM4 | −0.70 | −0.07 | 0.02 | 0.04 | 0.19 | −0.11 | −0.3 | 0.13 | 0.62 | 0.02 | −1.07 |
| LYST | 0.65 | −0.16 | −0.13 | 0.13 | −0.14 | −0.25 | 0 | −0.01 | −0.33 | −0.18 | −0.84 |
| MAN1A1 | 0.34 | 0.05 | −0.38 | 0.1 | −0.26 | 0.02 | −0.21 | −0.04 | −0.03 | −0.18 | −1.28 |
| MAN2B1 | −0.19 | −0.05 | −0.31 | −0.15 | 0.13 | −0.15 | −0.02 | 0 | −0.13 | −0.24 | −0.38 |
| MAOB | 0.15 | −0.02 | −0.14 | −0.28 | −0.16 | −0.2 | −0.14 | −0.17 | −0.09 | 0.1 | −1.27 |
| MAT1A | −0.16 | 0.12 | −0.82 | 0.15 | 0.3 | 0.72 | −1.09 | 0.07 | 0.16 | 0.02 | −1.39 |
| MBL2 | 0.24 | 0.22 | −0.91 | −0.6 | −0.47 | −1.06 | 0.4 | −0.29 | −0.71 | 0.23 | −0.11 |
| MCCC1 | 0.03 | 0.09 | −0.44 | −0.08 | 0.01 | −0.1 | −0.27 | −0.09 | 0.07 | 0.02 | −1.13 |
| MCCC2 | 0.26 | 0.09 | −0.48 | −0.36 | −0.2 | −0.45 | −0.45 | 0.07 | −0.15 | 0.02 | −0.43 |
| MCM6 | −0.12 | −0.11 | −0.84 | −0.22 | −0.05 | −0.55 | −1.06 | 0.32 | −0.3 | 0.02 | −0.84 |
| MEN1 | 0.21 | −0.03 | −0.46 | −0.14 | 0.15 | 0 | −0.18 | 0.09 | −0.21 | −0.25 | −0.01 |
| MET | 0.51 | 0.08 | −0.19 | −0.1 | −0.2 | −0.53 | −0.62 | 0 | −0.28 | 0.02 | −0.72 |
| MGAT2 | −0.34 | 0.00 | 0.14 | 0.03 | 0.09 | 0.12 | 0.07 | 0.11 | 0.14 | −0.23 | 0.27 |
| MKS1 | −0.15 | 0.06 | −0.32 | −0.24 | 0.17 | −0.26 | −1.9 | 0.04 | −0.09 | −0.11 | 0.1 |
| MLH1 | −0.13 | 0.05 | 0 | −0.03 | −0.06 | −0.39 | 0.33 | 0.01 | 0.25 | 0.12 | −0.24 |
| MMAA | −0.06 | 0.09 | −0.12 | −0.38 | −0.35 | −0.34 | −0.86 | 0.06 | −0.06 | 0.16 | −0.52 |
| MMAB | −0.69 | 0.04 | 0 | 0.85 | 0.27 | −0.35 | −0.52 | 0.02 | 0.2 | 0.07 | −0.68 |
| MMADHC | −0.34 | −0.06 | −0.08 | −0.13 | −0.24 | −0.23 | −0.78 | 0.22 | 0.42 | 0.12 | −0.12 |
| MOCS1 | −0.09 | 0.14 | −0.39 | 0.03 | 0.13 | 0.02 | −0.68 | 0.26 | 0.18 | 0.02 | 0.23 |
| MOGAT2 | 0.86 | −0.07 | −0.93 | −0.53 | −0.04 | −0.53 | −0.66 | −0.21 | −0.98 | 0 | −0.79 |
| MOGS | −0.23 | −0.04 | −0.19 | 0.04 | 0.24 | −0.12 | −0.48 | −0.01 | −0.15 | −0.14 | −0.2 |
| MPZ | −0.09 | 0.03 | −0.67 | 0 | 0.41 | −0.04 | −1.66 | 0.26 | −0.16 | 0.15 | −0.65 |
| MRPL3 | −0.10 | 0.00 | −0.28 | −0.29 | −0.06 | −0.6 | −1.32 | 0.38 | 0.36 | −0.03 | −0.68 |
| MTHFR | 0.16 | −0.04 | 0.34 | 0.61 | 0.35 | 0.59 | 0.87 | 0.04 | 0.22 | −0.12 | 1.05 |
| MTM1 | 0.15 | −0.11 | −0.02 | 0.04 | −0.13 | 0 | −0.02 | 0.06 | −0.08 | 0.09 | 0.09 |
| MTO1 | 0.00 | 0.05 | −0.1 | 0.17 | −0.03 | 0.07 | 0.22 | 0.07 | 0.43 | 0.08 | 0.19 |
| MTR | 0.75 | 0.15 | 0.23 | −0.16 | −0.08 | 0.25 | −0.87 | −0.12 | −0.39 | −0.02 | −0.79 |
| MTRR | 0.34 | −0.12 | −0.24 | −0.24 | −0.2 | −0.42 | −0.11 | 0.16 | 0.25 | 0.02 | −0.06 |
| MTTP | 0.29 | −0.04 | −0.17 | −0.04 | −0.29 | −0.16 | −1.29 | −0.15 | −0.54 | 0.17 | −1.04 |
| MVK | −0.58 | −0.01 | −0.27 | 1.44 | 0.29 | −0.28 | −0.94 | 0.05 | −0.02 | −0.01 | −1.29 |
| MYH9 | 0.45 | −0.09 | 0.17 | −0.28 | 0.02 | 0.16 | −0.82 | −0.36 | −0.1 | −0.17 | −2.29 |
| MYO5B | 0.43 | −0.06 | 0.25 | 0.39 | 0.2 | 0.08 | −0.18 | −0.09 | −0.01 | −0.08 | −1.08 |
| NAGS | −0.20 | 0.01 | 0.1 | 0.68 | 0.46 | 0.94 | 1.38 | 0.33 | 1.53 | −0.41 | −0.27 |
| NARS2 | −0.20 | 0.03 | −0.49 | −0.28 | −0.14 | −0.37 | −1.15 | −0.02 | 0.32 | −0.01 | −1.34 |
| NAT2 | −0.55 | 0.04 | −0.33 | −0.23 | −0.1 | −0.59 | −1.21 | 0.12 | 0.14 | 0.04 | 0.25 |
| NBAS | 0.41 | 0.01 | −0.07 | −0.25 | −0.22 | −0.21 | −0.79 | −0.34 | −0.32 | −0.07 | −2.46 |
| NCF2 | −0.18 | −0.11 | 0.85 | 0.51 | 0.47 | 0.17 | −1.74 | 0.79 | 0.59 | −0.04 | −1.06 |
| NEK1 | 0.01 | −0.04 | 0.14 | 0.19 | 0.1 | 0.15 | −0.67 | 0.11 | 0.29 | 0.18 | −0.75 |
| NEK8 | 0.35 | 0.08 | −0.22 | 0.2 | 0.29 | 0.3 | 1.71 | −0.15 | −0.04 | 0.04 | 0.41 |
| NEU1 | −0.55 | 0.03 | −0.35 | 1.01 | 0.51 | 0.18 | 0.72 | 0.25 | 0.04 | −0.11 | 0.62 |
| NFKB2 | −0.25 | 0.02 | −0.34 | −0.32 | 0.12 | 0.09 | −0.09 | 0.07 | −0.03 | −0.25 | −0.28 |
| NFKBIA | −0.38 | 0.08 | −0.37 | −0.57 | −0.06 | −0.27 | −1.35 | −0.2 | −0.22 | −0.08 | −0.62 |
| NFU1 | −0.61 | 0.00 | −0.11 | −0.05 | −0.04 | −0.45 | −0.12 | 0.1 | 0.5 | 0.2 | −0.37 |
| NGLY1 | −0.25 | −0.05 | −0.05 | −0.06 | −0.07 | −0.26 | −0.9 | 0.16 | 0.21 | −0.01 | −0.31 |
| NHP2 | −1.09 | −0.01 | −0.35 | −0.2 | 0.21 | −0.31 | −0.57 | 0.14 | 0.51 | −0.06 | −0.46 |
| NME1 | −1.01 | −0.15 | −0.83 | −0.19 | 0.06 | −0.81 | −0.18 | 0.31 | 0.59 | −0.07 | −0.17 |
| NOD2 | 0.33 | −0.10 | 0.31 | 0.15 | 0.33 | 0.16 | −0.58 | 0.07 | 0.2 | 0.12 | 0.12 |
| NOP10 | −0.83 | 0.04 | −0.32 | 0.1 | 0.2 | −0.37 | −0.58 | 0.24 | 0.43 | 0.03 | 0.26 |
| NOTCH1 | 0.55 | 0.03 | −0.31 | −0.06 | 0.16 | 0.15 | 1.73 | 0.12 | −0.35 | −0.74 | −0.27 |
| NOTCH2 | 0.92 | −0.06 | −0.11 | 0.06 | −0.03 | 0.03 | −0.61 | −0.14 | −0.67 | −0.14 | −1.28 |
| NPC1 | 0.40 | 0.01 | 0.46 | 0.89 | 0.3 | 0.47 | 0.15 | −0.1 | −0.1 | 0.03 | 0.16 |
| NR1H4 | 0.03 | 0.17 | −0.24 | 1.18 | 0.22 | 0.17 | −2.63 | −0.38 | 0.69 | 0.12 | −1.31 |
| NUBPL | 0.18 | 0.00 | −0.18 | −0.09 | −0.06 | −0.34 | −0.86 | −0.32 | 0.19 | 0.11 | −1.36 |
| NUDT12 | −0.03 | −0.01 | −0.25 | 0.19 | −0.17 | −0.08 | −1.1 | 0.17 | 0.13 | 0.15 | −0.01 |
| NUDT7 | −0.68 | 0.11 | −0.04 | −0.22 | −0.23 | 0.42 | −1.85 | −0.12 | 0.04 | 0.16 | −0.68 |
| OAT | −0.47 | 0.02 | 0.22 | 0 | 0.1 | 0.12 | 1.52 | 0.32 | 0.34 | −0.17 | 0.52 |
| OPA3 | 0.02 | −0.01 | −0.36 | −0.05 | 0.03 | −0.07 | −0.73 | −0.05 | −0.05 | −0.03 | −0.42 |
| OTC | −0.07 | −0.13 | −1.22 | −0.1 | −0.18 | −0.6 | 0.7 | 0.08 | −0.09 | 0.14 | −0.33 |
| PAF1 | −0.34 | −0.04 | −0.23 | −0.03 | 0.09 | −0.1 | −0.27 | 0.16 | −0.05 | −0.08 | 0.17 |
| PAH | 0.44 | 0.47 | −0.23 | 0.58 | 0.05 | 0.14 | −0.66 | −0.3 | 0.23 | 0.29 | −1.03 |
| PANK2 | −0.30 | 0.00 | 0.02 | −0.01 | −0.22 | −0.23 | 1.01 | 0.31 | 0.31 | 0.14 | 0.45 |
| PAOX | −0.38 | 0.04 | −0.37 | 0.07 | 0.16 | 0.07 | 0.54 | −0.18 | −0.3 | −0.54 | −1.01 |
| PC | 0.03 | 0.04 | −0.55 | 0.29 | 0.17 | 0.17 | 0.16 | −0.19 | −0.02 | −0.25 | −1.3 |
| PCCA | −0.09 | 0.11 | 0.19 | 0.36 | 0.13 | 0.13 | −0.09 | −0.34 | 0.37 | 0.05 | −1.26 |
| PCK2 | −0.68 | 0.06 | 0.58 | 0.51 | 0.46 | 1.32 | 0.57 | 0.06 | 1.77 | −0.06 | 0.68 |
| PCSK9 | 0.03 | −0.10 | 0.18 | 1.71 | 0.26 | −0.45 | 0.4 | −0.21 | −0.57 | −0.38 | −0.93 |
| PCYT1A | 0.37 | −0.05 | −0.25 | −0.19 | 0.09 | −0.06 | −0.42 | 0.16 | −0.18 | 0.01 | −0.25 |
| PDGFRA | 0.59 | −0.13 | 0.23 | −0.45 | −0.43 | −1.53 | 0.82 | 0.07 | −0.96 | 0.21 | −0.51 |
| PDP1 | 0.32 | −0.19 | −0.01 | 0.51 | −0.04 | −0.13 | 0.36 | −0.19 | −0.16 | −0.04 | −0.06 |
| PEPD | −0.47 | 0.02 | −0.7 | 0.48 | 0.32 | 0.04 | 0.01 | −0.02 | 0.17 | −0.29 | −1.36 |
| PEX11A | −0.15 | 0.00 | 0.24 | −0.13 | 0.02 | 0.39 | −0.79 | 0.24 | 0.06 | 0.15 | 0.28 |
| PEX12 | −0.09 | −0.06 | 0.3 | 0.03 | 0.11 | 0.04 | −1.18 | −0.12 | 0.34 | 0.04 | 0.5 |
| PEX14 | −0.23 | −0.03 | −0.21 | −0.32 | 0.15 | −0.16 | −0.85 | −0.23 | 0.27 | −0.32 | −1.12 |
| PFKM | −0.23 | 0.01 | −0.79 | −0.26 | 0.02 | −0.54 | 1.07 | 0.07 | −0.2 | −0.07 | −0.15 |
| PGM1 | −0.17 | 0.01 | 0.06 | −0.05 | 0.01 | 0.28 | −0.37 | 0.01 | 0.11 | −0.04 | −1.09 |
| PHGDH | −0.36 | 0.16 | 0.69 | 0.64 | 0.67 | 1.84 | −0.22 | 0.14 | 2.13 | −0.02 | 0.65 |
| PHKA1 | 0.02 | 0.02 | −0.43 | 0.34 | 0.42 | −0.07 | −0.14 | 0.26 | −0.06 | 0.13 | 0.4 |
| PHKA2 | 0.41 | 0.00 | −0.52 | −0.34 | −0.03 | −0.17 | −0.77 | −0.17 | −0.77 | 0.02 | −1.02 |
| PHKB | 0.23 | 0.09 | 0.21 | −0.07 | −0.06 | −0.11 | −0.83 | −0.05 | 0.01 | 0.01 | −1.4 |
| PHYH | −0.56 | −0.04 | −0.33 | 0.15 | −0.05 | −0.06 | 0.79 | 0.15 | 0.21 | 0.08 | 0.42 |
| PIEZO1 | 0.34 | −0.14 | 0.01 | −0.38 | 0.08 | 0.17 | −0.44 | −0.34 | −0.4 | −0.59 | −1.54 |
| PIGA | −0.07 | 0.21 | 0.98 | 0.24 | −0.01 | 0.23 | −0.47 | 0.11 | 0.59 | 0.13 | 0.51 |
| PIGM | −0.09 | −0.07 | −0.28 | −0.04 | 0.04 | −0.38 | −0.41 | 0.14 | 0.17 | 0.06 | 0.3 |
| PKD1 | 0.35 | 0.15 | 0.02 | 0.45 | 0.39 | 1.12 | 0 | −0.11 | 0.84 | −0.73 | −0.66 |
| PKD2 | 0.68 | −0.03 | 0.09 | −0.18 | −0.13 | 0.01 | −0.28 | −0.12 | −0.35 | −0.05 | 0.21 |
| PKHD1 | 1.14 | −0.20 | −0.65 | −1.1 | −0.79 | −0.21 | −1.8 | −1.08 | −1.08 | 0.06 | −2.91 |
| PKLR | −0.15 | −0.09 | −0.85 | 0.19 | 0.01 | −0.63 | −0.7 | −0.44 | −0.86 | 0.36 | −1.78 |
| PLA2G6 | 0.35 | 0.29 | 0.53 | 0.18 | 0.31 | 0.95 | 0.68 | −0.33 | 0.06 | 0.02 | −0.21 |
| PLEKHM1 | −0.03 | 0.08 | −0.47 | 0.77 | 0.52 | 0.39 | −0.08 | 0.33 | −0.03 | −0.11 | 0.1 |
| PMVK | −1.29 | 0.01 | −0.27 | 0.19 | 0.33 | −0.24 | −0.11 | 0.18 | 0.58 | −0.09 | −0.26 |
| PNP | −0.24 | −0.12 | −0.28 | 0.03 | 0.05 | −0.45 | 2.1 | 0.34 | 0.03 | −0.11 | 1.12 |
| PNPLA2 | −0.26 | 0.10 | 0.45 | −0.09 | 0.19 | 1.04 | 0.78 | 0 | 0.55 | −0.54 | −0.84 |
| POLD1 | −0.25 | −0.02 | −0.76 | −0.14 | 0.24 | −0.11 | −1.37 | 0.01 | −0.17 | −0.59 | −1.5 |
| POLG | 0.11 | −0.10 | −0.51 | −0.1 | 0.03 | −0.21 | 0.3 | 0.24 | −0.13 | −0.27 | 0.18 |
| POLG2 | −0.35 | 0.02 | −0.17 | −0.12 | 0.19 | 0.04 | 1.45 | −0.03 | −0.15 | 0.02 | 0.46 |
| POR | −0.48 | 0.82 | 0.28 | 0.46 | 0.82 | 1.32 | 0.36 | 0.08 | 0.31 | −0.24 | −0.32 |
| PPAPDC2 | 0.01 | 0.08 | −0.01 | 0.05 | −0.01 | −0.42 | 0.42 | 0.12 | 0.07 | −0.12 | 0.78 |
| PPARA | 0.80 | 0.07 | −0.12 | 0.33 | 0.09 | 0.44 | −0.47 | −0.01 | −0.35 | −0.06 | −0.8 |
| PPARG | −0.35 | −0.08 | 0.31 | 0.24 | 0.16 | −0.14 | −0.29 | −0.11 | 0.52 | 0.01 | −0.85 |
| PPM1K | −0.28 | −0.19 | −0.47 | 0.26 | 0.13 | −0.47 | 0.16 | 0.15 | 0 | 0.14 | −0.19 |
| PRKCD | 0.07 | 0.04 | −0.17 | −0.16 | 0.03 | 0.14 | 1.15 | 0.1 | −0.44 | −0.08 | 0.17 |
| PRODH | 0.19 | −0.41 | −1.33 | 0.22 | 0.2 | −0.2 | 3.9 | −0.01 | −0.57 | −0.12 | 2.13 |
| PROS1 | 0.17 | −0.14 | −0.3 | 0.07 | −0.19 | −0.33 | −0.04 | −0.09 | −0.22 | 0.05 | −0.65 |
| PROZ | −0.21 | 0.01 | −0.15 | 0.39 | 0.28 | 0.29 | 0.41 | −0.33 | −0.23 | 0.1 | 0.11 |
| PSAT1 | −0.01 | 0.15 | 0.7 | 0.52 | 0.26 | 1.13 | 0 | 0.28 | 2.35 | 0.15 | 0.14 |
| PSMB8 | −1.16 | −0.13 | −0.62 | −0.11 | 0.02 | −0.58 | 0.05 | 0.18 | 0.55 | 0.01 | 0.14 |
| PSPH | −0.08 | −0.07 | −0.44 | 0.56 | 0.05 | −0.23 | −0.62 | 0.09 | 0.89 | −0.06 | −0.55 |
| PTCH1 | 0.64 | −0.07 | −0.14 | 0.04 | 0.28 | 0.47 | 0.69 | 0.08 | −0.5 | 0.06 | −0.05 |
| PTRF | −0.21 | −0.25 | −0.58 | −0.32 | −0.15 | −0.65 | 0.32 | 0.26 | −0.07 | −0.4 | −0.67 |
| PTRH2 | −0.74 | 0.00 | −0.32 | −0.12 | 0.15 | −0.53 | −0.71 | 0.34 | 0.37 | 0.05 | 0.08 |
| PUS1 | −0.50 | −0.11 | −0.37 | −0.1 | 0.16 | −0.14 | −0.68 | 0.01 | 0.42 | −0.25 | −0.23 |
| PXMP2 | −0.64 | 0.12 | −0.23 | 0.09 | 0.19 | 0.13 | 0.16 | 0.03 | 0.36 | −0.12 | −0.43 |
| PXMP4 | 0.15 | −0.04 | −0.04 | 0.13 | 0.22 | 0.1 | −0.44 | 0.02 | −0.17 | 0.05 | −0.46 |
| RAI1 | 0.27 | −0.01 | −0.62 | −0.2 | 0.12 | −0.34 | −0.55 | −0.1 | −0.27 | −0.49 | −1.67 |
| RANBP2 | 0.53 | −0.01 | −0.06 | −0.2 | −0.29 | −0.31 | −0.43 | 0.16 | −0.28 | 0.02 | −0.06 |
| RASA1 | 0.34 | 0.00 | 0.42 | −0.11 | −0.13 | 0.07 | −0.17 | −0.14 | 0.12 | 0.02 | −0.5 |
| RBCK1 | −0.07 | 0.06 | 0.38 | 1.03 | 0.64 | 0.8 | −0.02 | 0 | 1.14 | −0.32 | 0.32 |
| RBM10 | −0.16 | 0.02 | −0.24 | −0.15 | 0.13 | 0.07 | −0.69 | 0.12 | −0.02 | −0.23 | −0.24 |
| REN | −0.46 | 0.07 | −0.26 | −0.39 | 0 | −0.91 | 1.98 | 0.36 | −0.37 | 0.02 | 0.57 |
| RMND1 | −0.03 | −0.06 | −0.26 | −0.14 | −0.07 | −0.5 | −1.31 | 0.23 | 0.03 | 0.19 | −0.35 |
| RMRP | −1.72 | −0.21 | −0.35 | 0.06 | 0.54 | 0.54 | 2.53 | 1.51 | −0.02 | −0.5 | −0.79 |
| RNASEH2A | −1.07 | 0.02 | −0.58 | 0.14 | 0.54 | 0.13 | −0.86 | 0.36 | 0.31 | −0.08 | −0.42 |
| RORC | 0.54 | 0.45 | 1.09 | −0.1 | 0.4 | 1.08 | −1.98 | −0.17 | 0.01 | 0.32 | −0.84 |
| RPGRIP1L | 0.41 | 0.11 | −0.03 | −0.52 | −0.2 | −0.55 | −1.64 | 0.12 | −0.23 | 0.17 | −0.44 |
| RRM2B | 0.33 | −0.01 | −0.22 | −0.3 | −0.53 | −0.67 | 1.48 | 0.26 | 0.2 | 0.02 | 0.79 |
| RSAD1 | −0.45 | 0.03 | −0.24 | −0.03 | 0.1 | 0.07 | −1.11 | 0.28 | 0.24 | −0.09 | 0.57 |
| SAMHD1 | 0.58 | −0.05 | −0.58 | −0.26 | −0.29 | −0.96 | 0.27 | 0.13 | −0.44 | −0.13 | −0.24 |
| SC5D | 0.08 | 0.02 | 0.5 | 1.03 | 0.05 | 0 | 0.1 | 0.15 | 0.21 | 0.09 | 0.89 |
| SCARB1 | −0.16 | 0.05 | −0.47 | −0.03 | 0.12 | −0.31 | 0.23 | −0.07 | −0.2 | −0.21 | −1.09 |
| SCN9A | 0.48 | −0.25 | 0.1 | −0.3 | −0.56 | −0.47 | −1.75 | −0.11 | −0.43 | −0.03 | −1.59 |
| SDCCAG8 | −0.03 | 0.11 | 0.27 | −0.11 | −0.12 | 0.02 | −0.55 | −0.19 | 0.35 | 0 | −1.32 |
| SEPN1 | 0.40 | 0.10 | −0.25 | −0.14 | 0.26 | −0.23 | −0.01 | −0.06 | −0.4 | −0.09 | −0.15 |
| SERAC1 | 0.19 | 0.17 | 0.01 | 0.06 | −0.1 | −0.2 | −0.39 | 0.01 | −0.21 | −0.1 | −0.66 |
| SERPINA1 | −0.30 | −0.01 | 0.41 | −0.06 | −0.03 | −0.03 | 0.63 | 0.05 | 0.31 | 0.17 | 0.31 |
| SERPING1 | −0.08 | 0.09 | 0.67 | −0.03 | 0.12 | 0.45 | 0.53 | −0.05 | 0.25 | 0.18 | 0.35 |
| SERPINI1 | −0.18 | −0.16 | 0.02 | 0.06 | −0.33 | −0.01 | 0.65 | 0.22 | 0.03 | 0.05 | 1.24 |
| SETBP1 | 0.46 | 0.02 | 0.23 | −0.23 | −0.1 | −0.11 | −0.08 | −0.32 | −0.44 | −0.17 | −1.3 |
| SFXN4 | −0.71 | 0.07 | −0.03 | −0.09 | 0.09 | −0.02 | −1.39 | 0.08 | 0.57 | 0.02 | −0.66 |
| SLC10A1 | 0.04 | 0.08 | −0.36 | −0.19 | −0.05 | −0.18 | 0.28 | −0.3 | −0.43 | 0.17 | −0.35 |
| SLC10A3 | −0.11 | 0.02 | −0.31 | −0.05 | 0.11 | −0.04 | 0.56 | 0.2 | 0 | −0.22 | 0.27 |
| SLC10A5 | 0.69 | −0.24 | −0.73 | −0.45 | −0.66 | 0.83 | −1.78 | −0.3 | −1.06 | 0.21 | −0.97 |
| SLC10A7 | 0.32 | −0.02 | 0.29 | −0.08 | −0.13 | 0 | −1.11 | −0.27 | 0.03 | −0.05 | −1.39 |
| SLC11A2 | 0.33 | 0.04 | 0.31 | 0.26 | 0.08 | 0.15 | −1.12 | −0.19 | −0.19 | 0.08 | −0.51 |
| SLC19A1 | −0.13 | 0.27 | −0.72 | −0.29 | 0.2 | −0.09 | −2.23 | 0.06 | 0.33 | −0.52 | −1.57 |
| SLC19A2 | 0.19 | −0.07 | 0.49 | 0.55 | 0.09 | 0.58 | 0.29 | 0.31 | 0.12 | −0.02 | 1.03 |
| SLC19A3 | −0.14 | 0.15 | −0.34 | −0.29 | 0 | −0.4 | −1.47 | −0.32 | −0.47 | 0.14 | −0.66 |
| SLC22A1 | −0.57 | 0.03 | −0.16 | −0.04 | 0.13 | 0.06 | 0.72 | 0.16 | 0.29 | −0.02 | 0.18 |
| SLC22A5 | 0.01 | −0.02 | −0.21 | 0.06 | 0.12 | −0.08 | −0.75 | 0.12 | 0 | −0.14 | −0.01 |
| SLC25A1 | −0.53 | −0.01 | −0.65 | 0.38 | 0.16 | −0.3 | 0.04 | 0.03 | −0.08 | −0.38 | −1.11 |
| SLC25A10 | −0.56 | 0.20 | −0.98 | −0.39 | 0.09 | −0.73 | −1.29 | 0.07 | −0.06 | −0.37 | −1.97 |
| SLC25A12 | −0.04 | −0.61 | −0.39 | −0.12 | 0.29 | −0.38 | −1.52 | −0.29 | 0.39 | 0.26 | −1.06 |
| SLC25A13 | 0.26 | 0.04 | −0.14 | 0.17 | 0.11 | −0.03 | −0.86 | −0.04 | −0.01 | 0.08 | −1.47 |
| SLC25A15 | −0.15 | 0.06 | −0.25 | 0.1 | −0.01 | −0.17 | −0.03 | 0.34 | 0.09 | 0.05 | 1.09 |
| SLC25A19 | −0.55 | 0.06 | −0.44 | −0.25 | −0.01 | −0.17 | 0.81 | 0.62 | 0.4 | −0.28 | 0.83 |
| SLC25A20 | −0.18 | −0.04 | −0.39 | −0.22 | −0.1 | 0.1 | 0.49 | 0.17 | 0.13 | 0.03 | −0.16 |
| SLC25A29 | −0.23 | 0.29 | −0.23 | 0.3 | 0.22 | 0.02 | 0.25 | −0.36 | −0.24 | −0.28 | −1.45 |
| SLC25A32 | −0.02 | −0.05 | −0.17 | −0.26 | −0.09 | −0.46 | −0.66 | 0.21 | 0.42 | −0.13 | −0.34 |
| SLC27A2 | −0.34 | 0.14 | 0.19 | −0.27 | −0.21 | −0.31 | −0.26 | −0.08 | 0.08 | −0.23 | −0.86 |
| SLC27A5 | −0.81 | 0.12 | −0.34 | 0.11 | 0.25 | 0.28 | 0.93 | 0.19 | 0.51 | −0.31 | 0.03 |
| SLC29A3 | 0.05 | 0.14 | −0.04 | −0.17 | 0.07 | −0.36 | −0.56 | −0.16 | −0.53 | 0.02 | −0.33 |
| SLC2A1 | −0.24 | −0.24 | −0.38 | 0.43 | 0.56 | −0.04 | 0.76 | 0.27 | 0.33 | −0.26 | −0.04 |
| SLC2A2 | 0.40 | 0.06 | −1.68 | −0.19 | −0.62 | −1.8 | −1.18 | −0.35 | −0.66 | 0.11 | −2.2 |
| SLC2A9 | 0.31 | 0.30 | −0.21 | 0.27 | 0.24 | 0.19 | 0.96 | −0.13 | 0.31 | −0.03 | −0.39 |
| SLC33A1 | 0.29 | 0.17 | 0.48 | 0.12 | 0.02 | −0.04 | −0.39 | 0.08 | 0.09 | −0.13 | −0.09 |
| SLC35C1 | −0.08 | 0.04 | −0.14 | 0.42 | 0.17 | 0.02 | −0.61 | 0.05 | 0 | −0.15 | −0.28 |
| SLC39A4 | −1.07 | 0.13 | −0.04 | 0.18 | 0.31 | −0.12 | −0.35 | 0.05 | 0.61 | −0.73 | −1.11 |
| SLC40A1 | 0.36 | 0.08 | 0.13 | 0.82 | 0.39 | 0.8 | −0.78 | 0.47 | −0.2 | 0.11 | −0.45 |
| SLC46A1 | 0.06 | 0.08 | −0.53 | −0.23 | 0.08 | −0.1 | −1.13 | 0.01 | −0.18 | −0.17 | −0.41 |
| SLC4A4 | 0.70 | 0.13 | −0.57 | −0.09 | −0.08 | −0.78 | −1.07 | −0.36 | −0.69 | 0 | −1.69 |
| SLC51A | −0.61 | −0.03 | −0.57 | −0.29 | 0.08 | −0.22 | −0.71 | 0.02 | −0.38 | 0.03 | −0.68 |
| SLC51B | −0.60 | 0.66 | −0.08 | 0.02 | 0.29 | −0.29 | 0.29 | 0.02 | −0.3 | 0.26 | 1.7 |
| SLC6A20 | −0.12 | 0.52 | −1.18 | 0.31 | 0.4 | −0.48 | 0.14 | −0.13 | −0.32 | 0.1 | −0.9 |
| SLC6A6 | −0.14 | 0.07 | −1.6 | 0.34 | 0.1 | −0.56 | −0.83 | 0.42 | −0.79 | −0.36 | −0.33 |
| SLC6A8 | −0.46 | −0.01 | −0.16 | 0.41 | 0.26 | 0.1 | 3.89 | 0.35 | −0.11 | −0.23 | 1 |
| SLC7A7 | −0.41 | −0.18 | −0.37 | −0.24 | −0.01 | −0.04 | −0.27 | −0.39 | −0.01 | −0.5 | −0.86 |
| SLCO1B1 | 0.09 | 0.10 | −0.1 | −0.19 | −0.33 | −0.03 | −1.05 | 0.19 | −0.18 | 0.06 | −1.71 |
| SLCO4C1 | 0.41 | 0.22 | 0.12 | 0.72 | −0.39 | −0.5 | −0.41 | −0.2 | −0.56 | 0.13 | −0.04 |
| SMAD4 | 0.10 | −0.06 | 0.05 | −0.02 | −0.2 | 0.01 | −0.4 | 0.04 | 0.03 | −0.01 | −0.19 |
| SMARCB1 | −0.36 | 0.06 | −0.58 | −0.3 | 0.11 | −0.37 | −1.3 | 0.06 | −0.03 | −0.07 | −0.83 |
| SNX10 | 0.19 | −0.08 | −0.04 | 0.21 | −0.36 | −0.19 | −1.26 | 0 | −0.02 | 0.11 | −1.48 |
| SORT1 | 0.42 | −0.13 | −0.15 | 0.13 | −0.15 | −0.32 | −0.4 | −0.09 | −0.43 | −0.09 | −1.05 |
| SPP2 | −0.92 | −0.24 | −0.24 | 0.01 | −0.12 | −0.26 | 1.13 | 0.23 | 0.58 | 0.11 | 0.4 |
| SPRTN | −0.04 | −0.10 | −0.42 | −0.21 | −0.17 | −0.55 | 0.59 | 0.26 | 0.01 | −0.12 | 0.52 |
| ST3GAL5 | −0.10 | 0.01 | −0.51 | 0.14 | −0.19 | −0.84 | −0.64 | −0.29 | −0.26 | −0.02 | −0.95 |
| STAT1 | 0.24 | −0.14 | −0.52 | −0.19 | −0.31 | −1.13 | 0.11 | 0.18 | −0.38 | −0.05 | −0.5 |
| STEAP3 | 0.12 | −0.07 | −0.22 | −0.21 | 0.03 | −0.56 | −0.16 | −0.01 | −0.42 | −0.13 | −0.66 |
| STIM1 | 0.13 | 0.02 | −0.22 | 0.13 | 0.1 | 0.17 | 0.52 | −0.19 | −0.04 | −0.11 | −1.09 |
| SULT2A1 | −0.10 | 0.44 | 0.29 | 0.85 | 0.65 | 1.88 | −0.14 | −0.35 | −0.13 | 0.39 | −1.07 |
| SUOX | −0.13 | 0.09 | −0.42 | −0.37 | −0.18 | −0.7 | −1.25 | −0.03 | −0.56 | 0.12 | −0.25 |
| TARS2 | −0.28 | 0.18 | −0.29 | −0.14 | 0.2 | −0.04 | −1.61 | 0.08 | 0.01 | 0.1 | −0.29 |
| TAT | 0.29 | 0.07 | 0.11 | −0.55 | −0.56 | 1.5 | −0.2 | −0.42 | 1.81 | 0.23 | −0.54 |
| TCIRG1 | −0.37 | −0.05 | −0.38 | 0.12 | 0.29 | 0.16 | −0.44 | −0.23 | −0.18 | −0.5 | −0.53 |
| TCTN3 | −0.07 | 0.04 | −0.24 | −0.27 | −0.04 | −0.28 | −0.15 | 0.03 | −0.01 | 0.13 | 0.18 |
| TDO2 | 0.08 | 0.09 | −1.49 | 0.89 | −0.58 | −1.61 | −0.77 | −0.78 | −0.6 | 0.36 | −2.8 |
| TF | −0.47 | −0.04 | 0.09 | 0.11 | 0.02 | 0.17 | 0.94 | 0.09 | 0.25 | 0.11 | 0.45 |
| TFR2 | 0.12 | −0.02 | −0.04 | 0 | 0.21 | 0.44 | 0.66 | 0.04 | 0.07 | −0.27 | −0.11 |
| TGFB1 | −0.17 | −0.22 | −0.2 | −0.37 | −0.25 | −0.88 | −0.06 | −0.21 | −0.24 | −0.35 | −1.52 |
| THPO | 0.07 | 0.09 | −0.46 | −0.06 | −0.03 | −0.48 | −0.03 | −0.03 | −0.03 | 0.09 | −0.3 |
| THRB | 0.43 | 0.00 | −0.11 | −0.18 | −0.28 | 0.34 | −0.27 | −0.2 | 0.44 | 0.11 | −1.33 |
| TK2 | 0.03 | 0.01 | −0.04 | 0.03 | 0.12 | −0.14 | −0.98 | −0.25 | −0.12 | 0.06 | −1.04 |
| TMEM135 | 0.11 | −0.10 | 0.25 | 0.38 | −0.13 | 0.04 | −0.62 | −0.33 | 0.07 | 0.16 | −1.74 |
| TMEM165 | −0.11 | −0.06 | −0.24 | −0.08 | −0.14 | −0.43 | 0.09 | 0.14 | 0.1 | −0.3 | −0.41 |
| TMEM67 | 0.04 | −0.18 | 0.18 | −0.29 | −0.01 | −0.51 | −1.04 | −0.1 | −0.14 | 0.08 | −0.44 |
| TMLHE | 0.33 | −0.06 | −0.25 | −0.38 | −0.03 | −0.12 | −0.51 | 0.01 | 0.04 | −0.09 | −1.31 |
| TMPRSS6 | −0.28 | 0.04 | −0.21 | −0.07 | 0.1 | 0.07 | 0.06 | −0.12 | 0.01 | −0.19 | −0.65 |
| TNFRSF1A | −0.16 | 0.05 | −0.5 | 0.04 | 0.25 | 0.05 | −0.17 | 0.12 | 0 | −0.16 | −0.44 |
| TNFSF11 | −0.46 | −0.06 | −0.63 | −0.39 | −0.54 | −0.9 | −1.71 | −0.08 | −0.57 | −0.34 | −1.42 |
| TP53 | 0.29 | 0.02 | −0.09 | −0.39 | −0.02 | −0.06 | −0.19 | 0.07 | 0.07 | 0.05 | 0.16 |
| TTC21B | 0.23 | −0.15 | 0.12 | −0.18 | −0.14 | 0.13 | 0.1 | 0.07 | −0.21 | 0.04 | −0.08 |
| TTPA | 0.32 | 0.12 | −0.6 | −0.51 | −0.46 | −0.39 | −1.46 | −0.23 | −0.69 | 0.16 | −1.43 |
| TTR | −0.78 | 0.00 | −0.16 | 0.04 | 0.19 | 0.05 | 0.55 | 0.12 | 0.41 | 0.15 | 0.25 |
| TYMP | −1.13 | −0.04 | −0.05 | 0.2 | 0.37 | 0.23 | −0.17 | 0.06 | 0.64 | −0.86 | −1.39 |
| UBR1 | 0.59 | 0.03 | −0.1 | −0.29 | −0.38 | −0.38 | −0.04 | 0.01 | −0.15 | −0.07 | −0.93 |
| UGT1A1 | 0.12 | 1.13 | −0.2 | −0.77 | −0.06 | 0.11 | −0.12 | −0.16 | −0.27 | 0.15 | −0.17 |
| UGT2B28 | −0.35 | 0.14 | −0.99 | −0.24 | 0.09 | −0.48 | 1.38 | 0.45 | −0.27 | 0.35 | 0.69 |
| UNC13D | −0.02 | 0.13 | 0.4 | −0.14 | 0.05 | 0.51 | −0.29 | −0.33 | −0.02 | −0.11 | 0.32 |
| UPB1 | −0.20 | 0.03 | −0.04 | 0.11 | 0.33 | 0.14 | 0.5 | −0.14 | −0.07 | 0.23 | −0.74 |
| UQCRB | −1.05 | 0.06 | −0.4 | −0.03 | 0.17 | −0.42 | 0.16 | 0.26 | 0.61 | 0.21 | 0.33 |
| UROC1 | −0.46 | 0.02 | −0.6 | −0.11 | −0.25 | −0.61 | 0.57 | 0.05 | 0.05 | −0.04 | −0.16 |
| UTRN | 1.01 | 0.28 | −0.11 | −0.29 | −0.35 | −0.29 | −0.58 | −0.64 | −0.51 | −0.09 | −3.47 |
| VIPAS39 | 0.15 | −0.01 | 0.05 | −0.07 | 0.05 | 0.1 | −0.64 | 0.16 | −0.05 | 0.14 | −0.04 |
| VLDLR | 0.11 | 0.59 | 0.88 | 0.57 | 0.62 | 0.68 | −0.75 | 0.06 | 1.33 | −0.08 | −0.47 |
| VPS13A | 1.19 | −0.03 | 0.21 | −0.08 | −0.28 | 0.09 | −0.65 | −0.28 | −0.49 | −0.04 | −1.79 |
| VPS33B | −0.21 | −0.25 | −0.86 | −0.1 | 0.18 | −0.44 | −0.4 | 0.06 | −0.05 | −0.11 | −0.11 |
| WAS | −0.09 | 0.13 | 0.08 | −0.22 | 0.19 | 0.26 | 0.4 | 0.21 | 0.18 | 0.48 | 1.27 |
| WDR35 | 0.62 | 0.06 | −0.28 | −0.46 | −0.19 | −0.23 | −1.05 | −0.06 | −0.12 | 0.01 | −0.7 |
| XDH | 0.49 | 0.46 | 1.07 | 0.53 | 0.28 | 0.66 | −0.01 | −0.25 | −0.13 | 0.19 | −0.19 |
| YARS2 | −0.34 | −0.16 | −0.56 | −0.18 | 0 | −0.42 | −0.36 | 0.23 | 0.17 | −0.21 | −0.08 |

Enzastaurin (41) was observed to significantly (q≤0.05) modulate expression of the following genes: ABCB11, ABCG8, ACOX2, ACTG2, ADCK3, AKR1C1, AKR1C2, AKR1C4, AKR1D1, ALAD, ALPL, ANGPTL3, AQP7, ASPA, BTD, C10orf10, CA2, CAV1, CDH1, CIDEC, CLDN1, CPT1A, CROT, CTDSP2, CYP39A1, DPYS, FGA, FGFR2, G6PC, GHR, H6PD, HAL, HMGCS2, HNF1B, HPGD, ID1, IDH2, IFNAR2, IYD, KRT18, KYNU, LIPC, MBL2, MMM, MTTP, MVK, NAGS, NOD2, NUDT7, PAH, PDGFRA, PGM1, PKHD1, PLA2G6, PLEKHM1, PNPLA2, PPARG, PTCH1, RMRP, RORC, SCN9A, SLC29A3, SLC2A2, SLC51A, SLCO1B1, TAT, TDO2, THRB, and TNFSF11.

BSI-201 (43) and peramivir (trihydrate) (46) were observed to significantly (q≤0.05) modulate expression of RMRP. Selumetinib (45) was observed to significantly (q≤0.05) modulate expression of the following genes: RMRP, SLC2A2, COL1A1, and FAM111B. Palifosfamide (47) was observed to significantly (q≤0.05) modulate expression of RMRP and FAM111B.

R788 (fostamatinib, disodium hexahydrate) (50) was observed to significantly (q≤0.05) modulate expression of the following genes: ABCC6, ACACB, AFP, AKR1D1, ALAD, ALDOB, APOB, AQP7, ARG1, ASPA, BMT, CA2, CA5A, CCND1, CPS1, CYP39A1, CYP3A4, EHHADH, FGA, G6PC, GYS2, HGD, HMGCS2, ID1, ITGB3, LPIN2, MBL2, NCF2, NOD2, NR1H4, PAH, PKLR, POR, PPARG, PRODH, RORC, SLC10A5, SLC25A15, SLC6A6, SLC7A7, SLCO4C1, SULT2A1, TAT, TGFB1, TNFSF11, and XDH. Torcetrapib (51) was observed to significantly (q≤0.05) modulate expression of the following genes: ACTG2, CYP3A4, ID1, KMT2D, and UTRN.

Tivozanib (52) was observed to significantly (q≤0.05) modulate expression of the following genes: AASS, ABAT, ABCA1, ABCB11, ABCC2, ABCC3, ABCD1, ABCD3, ABCG8, ABHD5, ACAD9, AFP, AGPS, AKR1C2, AKR1C4, ALDOA, ALG12, ANGPTL3, ANPEP, AP3B1, ARSB, ASNS, ATP7B, BCKDK, BCO1, C4A, CCND1, CIDEC, CLDN1, COL1A1, CTH, CYB5R3, CYP2C19, CYP7B1, DCDC2, DDO, DIS3L2, DLD, DNM1L, DPM3, ELAC2, FGA, FGD1, GBA, GGCX, GHR, GLDC, GPD1, GYS1, HGD, HTT, HYAL1, ID1, IL1RN, KCTD11, KRT18, KYNU, LAMB2, LDHB, LIPC, LMNB2, MAN2B1, MCCC2, MEN1, MMAB, MOGAT2, MOGS, MPZ, MTTP, NOTCH2, NR1H4, PCK2, PCYT1A, PDGFRA, PHGDH, PIGA, PKD2, PNP, POLD1, POLG, PROS1, PSAT1, PTRF, RBM10, RMRP, RORC, SAMHD1, SEPN1, SLC25A20, SLC27A2, SLC29A3, SLC2A2, SLC35C1, SLC4A4, SLC6A20, SLC6A6, SLCO1B1, SORT1, STAT1, STEAP3, SULT2A1, SUOX, TCTN3, TDO2, TGFB1, THPO, TMEM165, TMEM67, TNFRSF1A, TNFSF11, UBR1, UGT2B28, VPS33B, WDR35, and XDH.

17-MG (Tanespimycin) (53) was observed to significantly (q≤0.05) modulate expression of the following genes: ABCB11, ABCC2, ABHD5, ACACB, ACADSB, ACADVL, ACSL1, ACSL3, ADCK3, AK1, AKR1D1, ALAD, ALAS1, ALDOB, ALPL, AMPD3, ANKS4B, APOB, APRT, AQP7, ARG1, ARSA, ASNS, BMT, BBOX1, BRAF, CA12, CA2, CA5A, CCND1, CD320, CLDN1, COG6, CP, CPS1, CPT1A, CTH, CYP2B6, CYP39A1, CYP3A4, DBT, DHCR24, DHCR7, DMGDH, DPYD, EARS2, EDA, ENO3, ENPP1, EPGS, EPHX1, ERCC6, F5, FADD, FASLG, FGA, FGD1, FGFR2, G6PC, G6PC3, GALK1, GATA6, GCKR, GCLC, GLIS3, GNE, GYS2, H6PD, HGD, HLA-B, HMGCR, HMGCS2, HMOX1, HOGA1, HPS6, HRAS, HSD3B7, ID1, IDH1, IDUA, IFNAR2, IGFALS, IKBKG, INSR, IYD, KCTD11, KCTD7, KMT2D, LDLR, LPIN1, MBL2, MGAT2, MOCS1, MPZ, MVK, NFKB2, NFKBIA, NOD2, NOP10, NR1H4, NUDT7, PAH, PAOX, PCSK9, PDGFRA, PHGDH, PHKA1, PIEZO1, PMVK, PPARA, PSAT1, PSMB8, PTRF, PUS1, PXMP4, RORC, RRM2B, SC5D, SLC10A1, SLC10A3, SLC10A5, SLC25A29, SLC2A9, SLC39A4, SLC40A1, SLC6A6, SLC7A7, SLCO1B1, SLCO4C1, SULT2A1, TAT, TCIRG1, THPO, TNFSF11, TYMP, UPB1, UTRN, VLDLR, XDH

Zibotentan (54) was observed to significantly (q≤0.05) modulate expression of the following genes: ACOT8, APOA1, APOA2, APOB, APOE, APRT, COX6A1, CYP2C19, DCXR, DPM3, ETFB, FIS1, GCSH, HRAS, HSD17B10, KMT2D, NHP2, NME1, PKHD1, PMVK, PSMB8, RMRP, RNASEH2A, SLC39A4, TYMP, UQCRB, UTRN, and VPS13A. Semagacestat (55) was observed to significantly (q≤0.05) modulate expression of CYP3A4. Dalcetrapib (56) was observed to significantly (q≤0.05) modulate expression of CYP3A4 and GOSH. Preladenant (61) was observed to significantly (q≤0.05) modulate expression of the following genes: ABAT, ABHD5, ASNS, BCO1, CIDEC, CTH, DAO, DCDC2, DDC, FASLG, GCSH, GLDC, GPD1, HGD, HMGCS2, HOGA1, HSD3B7, ID1, KHK, LDLR, OTC, PRODH, RORC, SLC2A2, SLC6A6, TDO2, and XDH.

EVP-6124 (hydrochloride) (encenicline) (62) was observed to significantly (q≤0.05) modulate expression of the following genes: AKR1D1, DCDC2, DHCR7, G6PC, HMGCR, LDLR, LPIN1, MVK, NEU1, NR1H4, PCSK9, PKHD1, RBCK1, and SC5D. Vanoxerine (dihydrochloride) (65) was observed to significantly (q≤0.05) modulate expression of HMOX1. CO-1686 (Rociletinib) (66) was observed to significantly (q≤0.05) modulate expression of the following genes: ABAT, ABHD5, AKR1D1, ALPL, AQP7, ASNS, ASPA, CCND1, CPT1A, CTH, CYP3A4, DAO, DCDC2, G6PC, GLDC, GPD1, HBB, HMGCS2, ID1, IL1RN, INSR, LDHB, LPIN2, MBL2, PCK2, PDGFRA, PHGDH, PKD1, PNPLA2, POR, PSAT1, RORC, SLC2A2, STAT1, SULT2A1, TAT, and TDO2.

INNO-206 (aldoxorubicin) (70) was observed to significantly (q≤0.05) modulate expression of the following genes: AARS2, ABCD1, ACACA, ACACB, ACOX3, ACSF3, AFP, AGK, AGPAT2, AKR1C4, ALAD, ALAS1, ALDH1B1, ALMS1, AMPD3, ANKS4B, ARG2, ARSA, ATIC, ATM, BMT, BBOX1, BLNK, C10orf10, C10orf11, C15orf41, C9, CA2, CD46, CEP164, CEP290, CEP83, CFHR2, CPT2, CTNS, CYP2C19, CYP3A4, DCDC2, DHODH, DKC1, DLAT, DNAH5, ENO3, FAN1, FUCA1, FXN, G6PD, GCH1, GCKR, GLIS3, GLS2, GPC3, GTPBP3, GYS2, H6PD, HAL, HBB, HMBS, HNF1A, HNF4A, HRAS, IFNAR2, IFT122, IFT172, IL2RG, ISYNA1, KCTD11, LIPC, LRBA, MAT1A, MCM6, MKS1, MPZ, MRPL3, MTTP, NAGS, NARS2, NAT2, NCF2, NEK8, NFKBIA, NOTCH1, NR1H4, NUDT12, NUDT7, OAT, PANK2, PEX12, PFKM, PKHD1, PNP, POLD1, POLG2, PRKCD, PRODH, REN, RMND1, RMRP, RORC, RPGRIP1L, RRM2B, RSAD1, SCN9A, SFXN4, SLC10A5, SLC10A7, SLC11A2, SLC19A1, SLC19A3, SLC25A10, SLC25A12, SLC2A2, SLC46A1, SLC4A4, SLC6A8, SLCO1B1, SMARCB1, SNX10, SPP2, SUOX, TARS2, TMEM67, TNFSF11, TTPA, UGT2B28, and WDR35.

Pacritinib (SB1518) (75) was observed to significantly (q≤0.05) modulate expression of the following genes: A1BG, AARS2, ABCA1, ABCD1, ABCG5, ABCG8, ACADSB, ACOX2, ACTG2, ADA, ADSL, AGL, AGT, AGTR1, AGXT, AHI1, AHSG, AKR1D1, ALDH2, ALDH6A1, ALDOA, ALDOB, ALMS1, AMACR, AMPD3, AMT, ANKS4B, ANKS6, AOC2, AP3B1, APC, APOA1, APOA2, APOA5, APOC2, APOC3, APOE, AQP9, ARG1, ARL13B, ASL, ASPA, ATP7A, ATP7B, B2M, B9D2, BMT, BBS10, BCO1, BCR, BLOC1S3, BMPR1A, BRAF, BSCL2, C10orf10, C9, CA2, CCND1, CD81, CDAN1, CEP19, CEP290, CEP83, CFB, CFHR2, CHRM3, COG1, COGS, COG6, COL1A1, COX6A1, CP, CPT1A, CROT, CSPP1, CTC1, CTH, CTSF, CTSK, CYB5A, CYP27A1, CYP2B6, CYP2C19, CYP2D6, CYP3A4, CYP7B1, DCDC2, DCXR, DDO, DENR, DIS3L2, DLAT, DMGDH, DNM1L, DOLK, DPM3, ERCC4, ERCC6, ETHE1, F2, F9, FABP1, FADD, FAM111A, FAN1, FGA, FGD1, FGFR2, FIG4, FTH1, FXN, G6PC, G6PC3, G6PD, GALT, GATA6, GCH1, GHR, GJB2, GLDC, GNE, GNPTAB, GPC3, GPD1, GPHN, GTPBP3, GUSB, GYG1, GYS1, GYS2, H19, HBA1, HBB, HMBS, HMGCR, HNF1A, HNF1B, HNF4A, HPD, HPS4, HPS5, HPS6, HPX, HRAS, HRSP12, HYLS1, ID1, IFIH1, IFT140, IFT27, IFT80, ISYNA1, JAG1, JAK2, KCTD11, KIAA0586, KPTN, KRIT1, LCAT, LDLR, LDLRAP1, LIPC, LIPT1, LPA, LRBA, LYST, MEN1, MKS1, MLH1, MMM, MMADHC, MOCS1, MOGAT2, MOGS, MTM1, MTO1, MTR, MTRR, NAGS, NAT2, NCF2, NEK1, NFU1, NGLY1, NR1H4, NUDT7, OPA3, OTC, PAF1, PAOX, PCK2, PCSK9, PCYT1A, PDGFRA, PDP1, PEX11A, PHKA1, PHYH, PIGM, PKD2, PKHD1, PPAPDC2, PPARG, PPM1K, PRKCD, PROZ, PSPH, PTCH1, PTRH2, PXMP2, PXMP4, RAH, RANBP2, RASA1, RMND1, RMRP, RPGRIP1L, SCN9A, SERAC1, SERPINA1, SERPING1, SLC10A1, SLC10A3, SLC10A7, SLC19A1, SLC19A3, SLC22A1, SLC22A5, SLC25A12, SLC25A19, SLC25A32, SLC27A5, SLC29A3, SLC2A1, SLC2A2, SLC2A9, SLC33A1, SLC6A20, SMAD4, SMARCB1, SPP2, SPRTN, ST3GAL5, SUOX, TARS2, TAT, TDO2, TF, TFR2, THPO, TK2, TMPRSS6, TNFRSF1A, TNFSF11, TP53, TTC21B, TTR, UBR1, UGT1A1, UNC13D, UROC1, UTRN, VIPAS39, VLDLR, VPS13A, VPS33B, WDR35, XDH, and YARS2.

TH-302 (Evofosfamide) (76) was observed to significantly (q≤0.05) modulate expression of the following genes: FAM111B, FGFR2, PKHD1, and RMRP. TP-434 (Eravacycline) (79) was observed to significantly (q≤0.05) modulate expression of the following genes: ABAT, ABHD5, AKR1D1, ASNS, CTH, HAX1, HMGCS2, ID1, NAGS, PCK2, PHGDH, PKHD1, PSAT1, RBCK1, SLC10A5, TAT, and VLDLR. NOV-002 (oxidized L-Glutathione) (83) was observed to significantly (q≤0.05) modulate expression of GOSH and IGFALS.

Bms-986094 (inx-189) (84) was observed to significantly (q≤0.05) modulate expression of the following genes: ABAT, ABCC2, ABCC6, ACACA, ACACB, ACOX3, ACTN1, ADK, AGPAT2, AGPS, AKR1C2, AKR1C4, ALMS1, ALPL, AP3B1, ARG1, ASNS, ASPA, ASXL1, ATG7, ATP8B1, BMT, BCKDHB, BCR, BLNK, C10orf11, C15orf41, CA5A, CDH1, CHRM3, COG5, CPT1A, CPT2, CTH, CTSK, CYP2C19, CYP7B1, DCDC2, DHCR24, DIS3L2, DNAH5, DPYD, DPYS, EDA, F2RL1, FARS2, FGD1, FGFR2, FTCD, FXN, G6PD, GALK2, GATM, GBE1, GCSH, GHR, GLDC, GLIS3, GLS2, GLYCTK, GPHN, H6PD, HBB, HMGCS2, HMOX1, HNF1B, HNF4A, HOGA1, HTT, ID1, IFNAR2, IFT122, IFT140, IGFALS, IKBKG, IL1RN, INSR, INVS, ITGB3, KYNU, LIPC, LRBA, LRP5, LYRM4, MAN1A1, MAOB, MAT1A, MCCC1, MTHFR, MTTP, MVK, MYH9, MYO5B, NARS2, NBAS, NCF2, NOTCH2, NR1H4, NUBPL, PAH, PAOX, PC, PCCA, PEPD, PEX14, PGM1, PHKA2, PHKB, PIEZO1, PKHD1, PKLR, PNP, POLD1, PRODH, RAI1, SCARB1, SCN9A, SDCCAG8, SERPINI1, SETBP1, SLC10A7, SLC19A1, SLC19A2, SLC25A1, SLC25A10, SLC25A12, SLC25A13, SLC25A15, SLC25A29, SLC2A2, SLC39A4, SLC4A4, SLC51B, SLC6A8, SLCO1B1, SNX10, SORT1, STIM1, SULT2A1, TDO2, TGFB1, THRB, TK2, TMEM135, TMLHE, TNFSF11, TTPA, TYMP, UTRN, VPS13A, and WAS. TC-5214 (R-Mecamylamine hydrochloride) (85) was observed to significantly (q≤0.05) modulate expression of GCSH, which had a log 2 fold change in expression of −1.31.

The following compounds were observed to have no effect on any disease gene expressional) DMXAA (42), which targets DT-diaphorase, a potent tumor blood vessel-disrupting agent; 2) dalcetrapib (56), which targets cholesteryl ester transfer protein increasing plasma HDL; 3) latrepirdine (57), which is a potent activator of AMPK that promotes the removal of α-synuclein; 4) CMX001 (58), which targets certain herpesviruses, adenoviruses, and orthopoxviruses; and 5) temsirolimus (60), which targets mTOR to suppress proliferation of a broad panel of tumor cells.

In fact, DMXAA (42), Evacetrapib (48), Latrepirdine (dihydrochloride) (57), CMX001 (Brincidofovir) (58), Vicriviroc (maleate) (59), Temsirolimus (60), Bitopertin (63), Latrepirdine (dihydrochloride) (64), Laropiprant (tredaptive) (67), Bardoxolone (68), VX-661 (tezacaptor) (69), LY404039 (pomaglumetad methionil (mGlu2/3) (71), Perifosine (KRX-0401) (72), Cabozantinib (XL184, BMS-907351) (73), Dacomitinib (PF299804, PF299) (74), a-PHP (77), LY 2140023 (Pomaglumetad methionil-LY404039) (78), TC-5214 (S-(+)-MecaMylaMine Hydrochloride) (80), Rolofylline (KW-3902) (81), and Amigal (Deoxygalactonojirimycin hydrochloride) (82) were not observed to significantly modulate expression (p≤0.05) of any of the genes analyzed.

B. Conclusions

Darapladib (44) was observed to significantly (q≤0.05) modulate expression of the following genes: RMRP, AKR1C4, AKR1D1, ANGPTL3, ID1, MBL2, NAGS, NOD2, PLEKHM1, AFP, DCDC2, DHCR7, HMGCR, IL1RN, LDLR, NPC1, NR1H4, PCSK9, and RBCK1. Darapladib was used as a positive control. Darapladib inhibits Lp-PLA2 and was considered as a possible add-on treatment for atherosclerosis. In a clinical trial, darapladib failed to reduce the risk of coronary heart disease death, myocardial infarction, and urgent coronary revascularization. The RNA-seq results herein show that the compound up-regulated genes: LDLR, PCSK9, and HMGCR (Hypercholesterolemia). HMGCR is a target of statins to reduce LDL concentration in plasma. The upregulation of HMGCR may explain the compound's lack of efficacy. ANGPTL3 was observed to be down-regulated, which is known to reduce plasma LDL levels.

Cediranib (49) was observed to significantly (q≤0.05) modulate expression of the following genes: RMRP, SLC2A2, ANGPTL3, ID1, MBL2, DCDC2, LDLR, ABCB11, ACOX2, CA2, CLDN1, DPYS, FGA, FGFR2, GHR, HAL, LIPC, MTTP, MVK, PDGFRA, SLCO1B1, TDO2, TNFSF11, TTPA, ABAT, ABCA1, ABCB4, BCHE, CCND1, DDC, F5, GLDC, LPIN1, MET, SAMHD1, SLC27A2, and SULT2A1.

Cediranib is known to inhibit VEGFR2, which inhibits VEGF-stimulated proliferation of tumor cells. In a clinical trial, the compound faded to meet its primary endpoint and survival of glioblastoma patients was not extended. Cediranib was observed to down-regulate Factor V levels in the RNA-seq results herein although VEGFR2 is not expressed in hepatocytes.

Factor V Leiden Thrombophilia is an autosomal dominant disease affecting between 3-8% of the Caucasian population, who carry one copy of the mutation. The presence of one copy of the Factor V Leiden mutation increases disease risk two-fold. About 1 in 5,000 mostly Caucasian people have two copies of the mutation, which raises the risk of disease 21-fold. Results herein show that Cediranib may be potentially effective to treat Factor V Leiden Thrombophilia.

Five compounds were observed to have an effect on expression of too many disease genes: 1) enzastaurin (41), which targets PKC resulting in a pro-apoptotic and anti-proliferative effect on tumor cells; 2) fostamatinib (50), which targets SYK resulting in an anti-proliferative effect on blood tumor cells; 3) tivozanib (52), which targets VGFR1/2/3 to block tumor growth in xenograft models; 4) cediranib (49), which targets VGFR2 resulting in an anti-proliferative effect on tumor cells; and 5) 17-MG (53), which targets HSP90 resulting in a pro-apoptotic and anti-proliferative effect on tumor cells.

### Example 5. Significant Effects of Phase III Failure Compounds on Gene Expression

In the RNA-seq results of Table 36, 15 compounds did not cause significant changes in gene expression. Significance was determined by the criteria of FPKM≥0.5, a log 2(fold-change)≥1, and a q-value≥0.05. Table 37 shows the number of expression significantly genes resulting from administration of the 45 Phase III failures chosen for analysis herein.

TABLE 37
Number (#) of genes having significantly changed expression

| Compound | # of genes | Compound | # of genes | Compound | # of genes |
|---|---|---|---|---|---|
| Enzstaurin | 971 | Zibotentan | 505 | TH-302 | 28 |
| BSI-201 | 7 | Semagacestat | 9 | TP-434 | 232 |
| Darapladib | 264 | Dalcetrapib | 43 | NOV-002 | 54 |
| Selumetinib | 60 | Latrepirdine | 12 | bms-986094 | 2079 |
| Peramivir | 23 | Temsirolimus | 34 | TC-5214 | 30 |
| Palifosfamide | 15 | Preladenant | 317 | | |
| Cediranib | 349 | EVP-6124 | 141 | | |
| Fostamatinib | 560 | Vanoxerine | 17 | | |
| Torcetrapib | 78 | CO-1686 | 391 | | |
| Tivozanib | 1510 | INNO-206 | 1944 | | |
| 17-AAG | 1558 | Pacritinib | 4114 | | |

DMXAA (42), evacetrapib, CMX001 (brincidofovir), vicriviroc (maleate), bitopertin, latrepirdine (dihydrochloride), laropiprant (tredaptive), VX-661 (tezacaptor), LY404039 (pomaglumetad methionil (mGlu2/3)), cabozantinib (XL184, BMS-907351), a-PHP, LY 2140023 (Pomaglumetad methionil-LY404039), TC-5214 (S-(+)-MecaMylaMine Hydrochloride), rolofylline (KW-3902), Amigal (Deoxygalactonojirimycin hydrochloride), and TC-5214 (R-Mecamylamine hydrochloride) were not observed to cause any significant changes in gene expression.

Table 38 shows the number of genes having significantly changed expression for each compound in the pilot RNA-seq study described in Example 2. Significance is defined as an FPKM≥0.5 a log 2(fold change)≥1, and a q-value of ≤0.05.

TABLE 38
Number (#) of genes having significantly changed expression

| Compound | # of genes | Compound | # of genes | Compound | # of genes |
|---|---|---|---|---|---|
| Simvastatin | 26 | Prednisone | 247 | Ibuprofen | 18 |
| Danazol | 5 | Penicillamine(D-) | 9 | Propylthiouracil | 8 |
| Nefazodone | 18 | Disopyramide | 7 | Rapamycin | 67 |
| Rosiglitazone maleate | 78 | Rifampicin | 90 | BIO | 82 |
| Sulpiride | 8 | Benzbromarone | 119 | ATRA | 30 |
| Captopril | 12 | Isoniazid | 14 | Xav939 | 8 |
| Imatinib | 31 | Acetaminophen | 3 | Bms189453 | 1 |
| Papaverine | 12 | Ritonavir | 183 | Dorsomorphin | 15 |
| Amiodarone | 19 | SGI-1776 | 64 | BMP2 | 5 |
| Nitrofurantoin | 107 | Valproate | 4 | Bms777607 | 19 |
| | | | | Bms833923 | 66 |

About 64% of the Phase III failure compounds were observed to significantly affect gene expression compared to about 80% of the first 39 compounds analyzed in the pilot RNA-seq analysis. The RNA-seq also showed that more genes (7597 vs. 703) were significantly affected by the Phase III failure compounds than the pilot compounds. Several of the Phase III failure compounds significantly affected more than 1000 genes, such as HSP90 inhibitor, FLT3 and JAK2 inhibitor, and NS5B RNA polymerase inhibitor. Five hundred forty-three of 1150 liver disease genes showed significant changes in gene expression in response to at least one Phase III failure compound.

### Example 6. Analyzing the Frequency of Identifying Features of Enhancers

Enhancers have previously been defined by the following annotations: H3K27ac+ and/or H3K4me1+(Active), H3Kme1+/H3K27ac−(Poised), p300, Brd4, super enhancers, and a cluster of master transcription factors (e.g., HNF3b, HNF4a, HNF6, HNF4, ATFS, 002, SMC, and YY1). The counts of these features in hepatocytes as determined by ChIP-seq are shown in Table 39.

TABLE 39
Feature counts in hepatocytes

| Type | Total | Non-promoter | Type | Total | Non-promoter |
|---|---|---|---|---|---|
| SP clusters | 102,345 | 85,990 | p300 | 9,217 | 7,770 |
| H3K27ac | 37,733 | 24,474 | Brd4 | 49,315 | 37,377 |
| H3K27ac+/H3K4me1+ | 9,997 | 8,442 | SuperEnhancers | 461 | 215 |
| H3K27ac−/H3K4me1+ | 21,353 | 21,153 | masterTF clusters | 206,548 | 188,516 |

As shown above, the majority of enhancers were observed in non-promoter regions indicating the importance of the three-dimensional architecture of the genome to interactions between enhancers and promoters.

### Example 7. Identifying Overlap Between Signaling Proteins and Enhancers

Signaling proteins bind to an enhancer to after the three-dimensional architecture of the genome and mediate gene looping resulting in the interaction between enhancers and promoters.

Table 40 shows the ChIP-seq results in hepatocytes showing naïve overlap of signaling proteins and enhancers.

TABLE 40
Naïve overlap between signaling proteins and enhancers

| Number of distinct | Overlap | Enhancers without | |
|---|---|---|---|
| signaling proteins | sites | signaling proteins | Annotation |
| 69,034 | 24,466 | 3,636 | H3k27ac |
| 88,163 | 9,456 | 437 | H3k27ac+/ |
| | | | H3K4me1+ |
| 91,307 | 9,751 | 9,953 | H3k27ac−/ |
| | | | H3Kme1+ |
| 93,883 | 8,421 | 294 | p300 |
| 56,949 | 39,053 | 8,093 | Brd4 |
| 98,171 | 461 | 0 | Super enhancers |
| 27,176 | 68,168 | 134,155 | Master |
| | | | transcription |
| | | | factor |
| | | | clusters |

Nearly all regions with H3k27ac, H3k27ac+/H3k4me1+, and p300 were also observed to have bound signaling proteins. No clear bias was observed for regions with H3K27ac-/H3K4me1+. Regions bound with Brd4 often were observed to also have at least one signaling protein. AM regions with super enhancers were observed to also have signaling proteins. Signaling proteins were observed to generally bind regions containing master transcription factor (TF) clusters. Insulated neighborhoods (INs) that did not contain a gene were also identified. The results herein show that an occupancy-dependent signaling center (ODSC) is defined by a region of the genome bound by signaling proteins and a H3K27 chemical modification, and independently includes at least one of a bromodomain-containing protein (Brd), a transcriptional coactivator (e.g. p300), and master transcription factors bound to the region.

### Example 8. Determining Genomic Position and Composition of GSCs

A multilayered approach is used herein to identify locations or the “footprint” of GSCs. The linear proximity of genes and enhancers is not always instructive to determine the 3D conformation of the genomic signaling centers.

ChIP-seq was used to determine the genomic position and composition of GSCs. Antibodies specific to 57 targets, including transcription factors, signaling proteins, and chromatin modifications, were selected for validation in HepG2 cells using ChIP-seq. These validated antibodies were used in ChIP-seq for hepatocytes to create a two-dimensional (2D) map. These antibody targets are shown in Table 41.

TABLE 41
ChIP-seq targets for primary human hepatocytes

| Chromatin Markers | Transcription factors | | Signaling proteins | |
|---|---|---|---|---|
| H3K4me3 | ATF5 | RNA Pol II | STAT1 - JAK/STAT | NR3C1 (glucocorticoid receptor, GR) |
| H3K27Ac | FOXA1 | ONECUT2 | STAT3 - JAK/STAT | ATR - DNA damage response |
| H3K4me1 | HNF4A | PROX1 | p53 - p53, mTOR, AMPK | ESR1 (estrogen receptor, ERa) |
| H3K27me3 | NROB2 | MLXIPL | TEAD 1/2 - Hippo | NR1H3 (liver X receptor, LXRa) |
| p300 | FOXA2 | YY1 | NF-κB (RelA/p65) - NF-κB | NR1H4 (farnesoid X receptor, FXR) |
| BRD4 | CUX2 | CTCF | CREB1 - MAPK | AHR (aryl hydrocarbon receptor) |
| SMC1 | HHEX | HNF6 (ONECUT1) | NR5A2 (LRH-1) | WWTR1 - Hippo |
| | ZGPAT | HNF1A | c-JUN - TLR, MAPK | HIF1a (hypoxia inducible factor) |
| | NR113 | C-MYC | c-FOS - TLR, MAPK | TCF7L2 (TCF4) - WNT |
| | | | ELK1 - MAPK | NR1I2 (pregnane X receptor, PXR) |
| | | | SMAD2/3 - TGFβ | RBPJK - NOTCH |
| | | | SMAD4 -TGFβ | SREBP1 - sterol |
| | | | SMAD1/5/8 - TGFβ | SREBP2 - sterol |
| | | | MTOR - mTOR | STAT5 - JAK/STAT |
| | | | RARalpha - RA pathway | RXR - RA pathway |
| | | | NR1I1 (VDR) | YAP1 - Hippo |

In the signaling proteins column, the associated canonical pathway is included after the “-”.

A. Binding Profiles and Gene Expression in Non-Perturbed Cell Systems

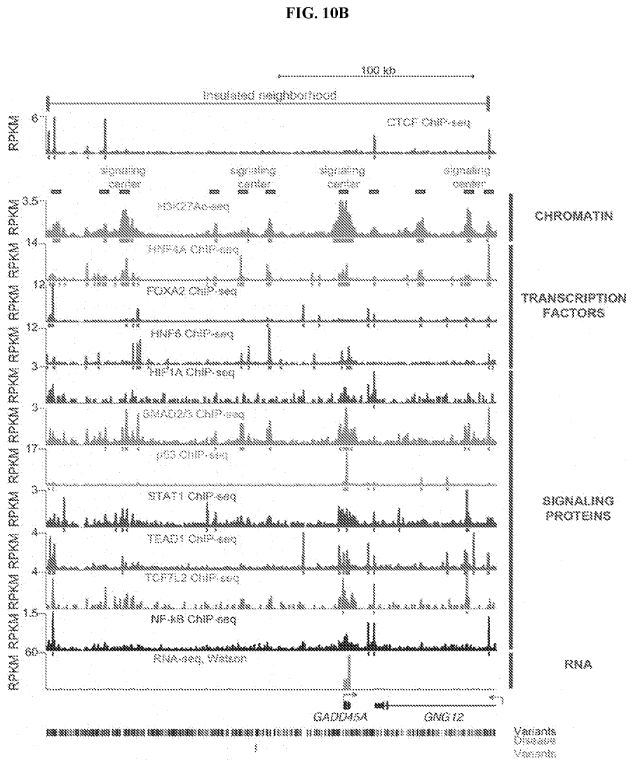

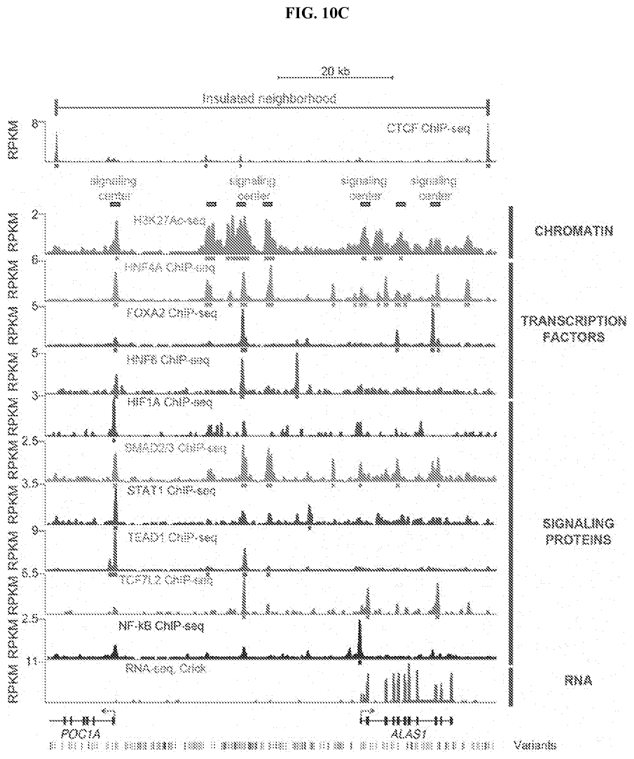

ChIP-seq and RNA-seq were performed as described in Example 1 on primary human hepatocytes. Results are shown in FIG. 10A for an insulated neighborhood containing the DNMBP gene. Transcription start sites for two neighboring genes ABCC2 and CPN1 were located outside of the insulated neighborhood boundary. Therefore, these two genes did not belong to the same insulated neighborhood as DNMBP. The RNA-seq signal peaks on the gene tracks reflect expression levels for the three genes.

Positions of the six GSCs within the IN were determined partially based on presence of H3K27Ac chromatin modification. Transcription factor HNF4A and signaling proteins SMAD2/3, TP53, STAT1, TEAD1, and NF-kB were all observed to bind the highlighted genomic signaling center adjacent to the CPN1 gene. SNP disease variant rs10883437 was also observed to be located in the highlighted GSC.

Eleven GSCs in the insulated neighborhood containing GADD45A gene were occupied cumulatively by transcription factors HNF4A, FOXA2, and HNF6, and signaling proteins HIF1A, SMAD2/3, TP53, STAT1, TEAD1, TCF7L2, and NF-kB, as shown in FIG. 10B. Positions of the GSCs within the neighborhood were determined partially based on presence of H3K27Ac chromatin modification. RNA-seq signal on the tracks reflects expression levels for the GADD45A gene. Known disease variants are shown below the RNA-seq tracks in FIG. 10B.

FIG. 10C shows an insulated neighborhood containing the POC1A gene as well as liver disease gene ALAS1. The seven genomic signaling centers in the neighborhood were occupied cumulatively by transcription factors HNF4A, FOXA2, and HNF6, and signaling proteins HIF1A, SMAD2/3, STAT1, TEAD1, TCF7L2, and NF-κB. The RNA-seq signal on the gene tracks reflects expression levels for the ALAS1 gene.

B. Binding Profiles and Gene Expression in Perturbed Cell Systems

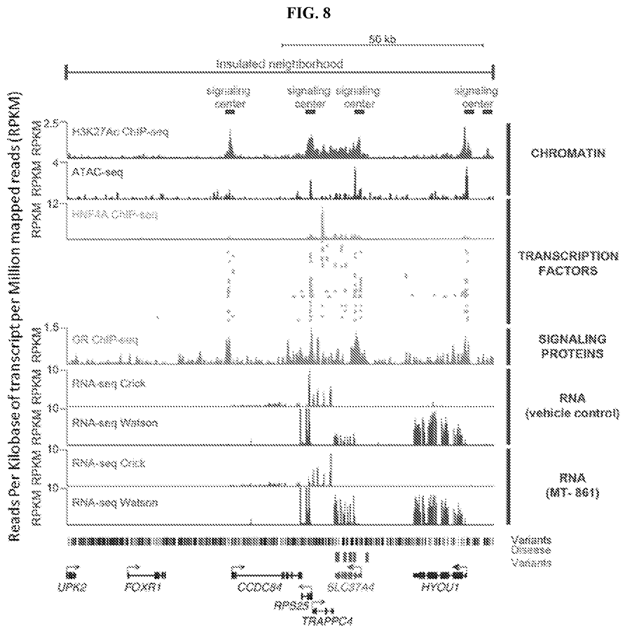

As shown in Table 33, prednisone, an immunosuppressant, was observed to upregulate the SLC37A4 gene, which is associated with glycogen storage disease 1b. ChIP-seq and RNA-seq results are shown in FIG. 8 for an insulated neighborhood containing FOXR1, CCDC84, RPS95, TRAPPC4, SLC37A4, and HYOU1 genes. The five genomic signaling centers in the neighborhood were observed to be occupied cumulatively by transcription factor HNF4A and signaling protein Glucocorticoid Receptor (GR). Positions of the GSCs within the neighborhood were determined based on presence of H3K27Ac chromatin modification and an ATAC-seq. RNA-seq signal on the tracks reflects changes in expression levels for all the genes in the IN.

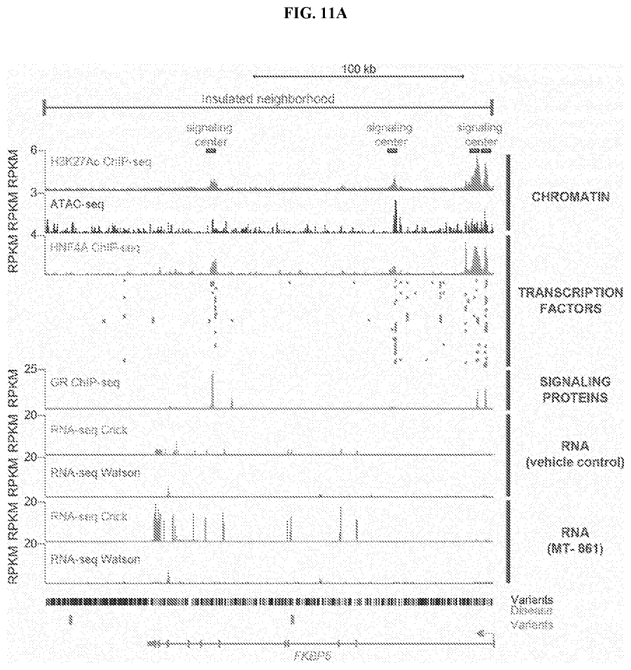

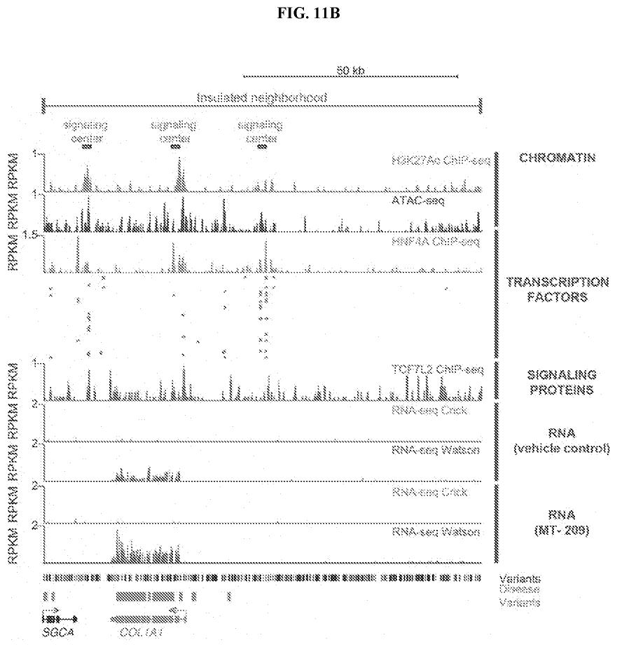

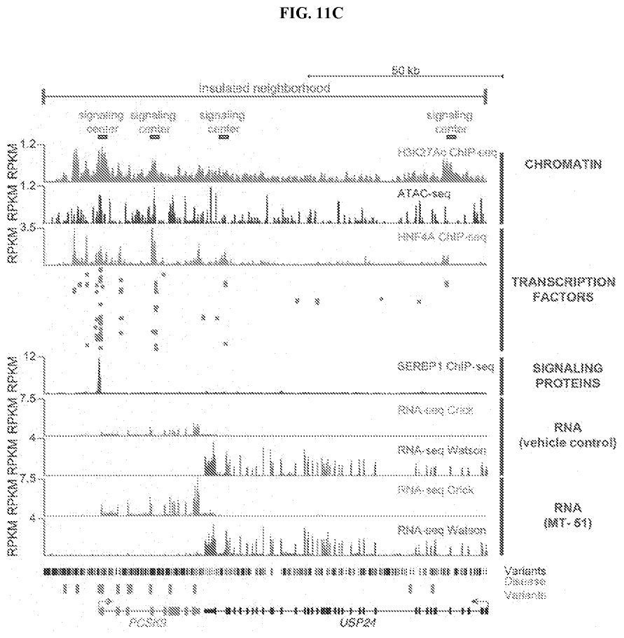

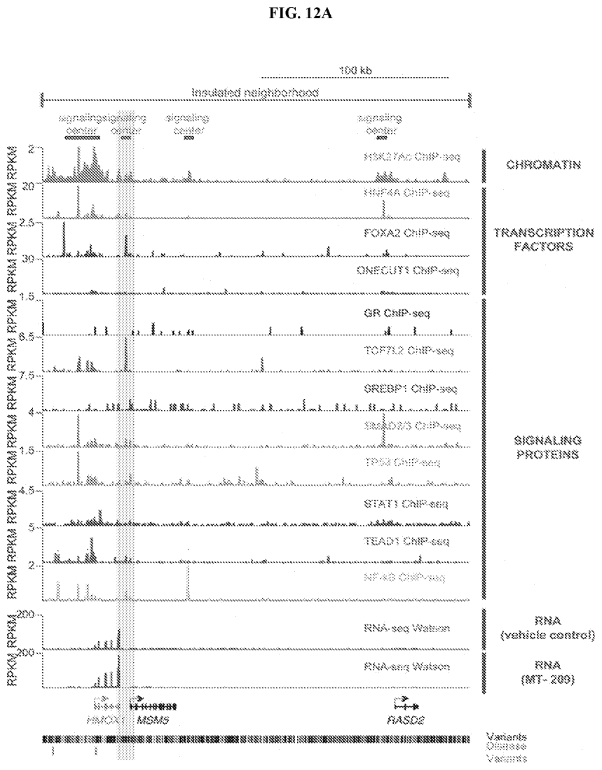

The RNA-seq results in FIG. 8 and in Table 33 confirm that SLC37A4 was significantly upregulated in cells treated with prednisone compared to cells treated with a vehicle control. Known disease variants are shown below the RNA-seq tracks in FIG. 8. The steroid drug, prednisone, was also observed to also upregulate FKPP5, a gene associated with immunosuppression, in a different IN than the neighborhood including SLC37A4, as shown in FIG. 11A. Known disease variants are shown below the RNA-seq tracks in FIG. 11A.

FIG. 11B also shows that BIO, a Wnt pathway agonist, upregulated expression of the COL1A1 gene implicated in liver fibrosis. The three GSCs in the IN containing COL1A1 are occupied cumulatively by transcription factor HNF4A and signaling protein TCF7L2. Positions of the three GSCs within the IN were determined partially based on presence of H3K27Ac chromatin modification and ATAC-seq signal. RNA-seq signal on the gene tracks reflects expression levels for the COL1A1 gene. The RNA-seq results shown on the gene tracks in FIG. 11B and in Table 33 confirm that COL1A1 was significantly up-regulated in cells treated with BIO in comparison to cells treated with vehicle control. Known disease variants are shown below the RNA-seq tracks in FIG. 11B.

Simvastatin, an inhibitor of HMG-CoA reductase and a key enzyme in cholesterol biosynthesis pathway, up-regulated PCSK9, a gene implicated in familial hypercholesterolemia. The four GSCs in the IN containing the PCSK9 and USP24 genes are occupied cumulatively by transcription factor HNF4A and signaling protein SREBP1. Positions of the four GSCs within the IN were determined partially based on presence of H3K27Ac chromatin modification and ATAC-seq signal. RNA-seq signal on the gene tracks reflects expression levels for PCSK9 and USP24 genes. The RNA-seq results shown on the gene tracks in FIG. 11C and in Table 33 confirm that PCSK9 was significantly up-regulated in cells treated with simvastatin in comparison to cells treated with a vehicle control. Known disease variants are shown below the RNA-seq tracks in FIG. 11C.

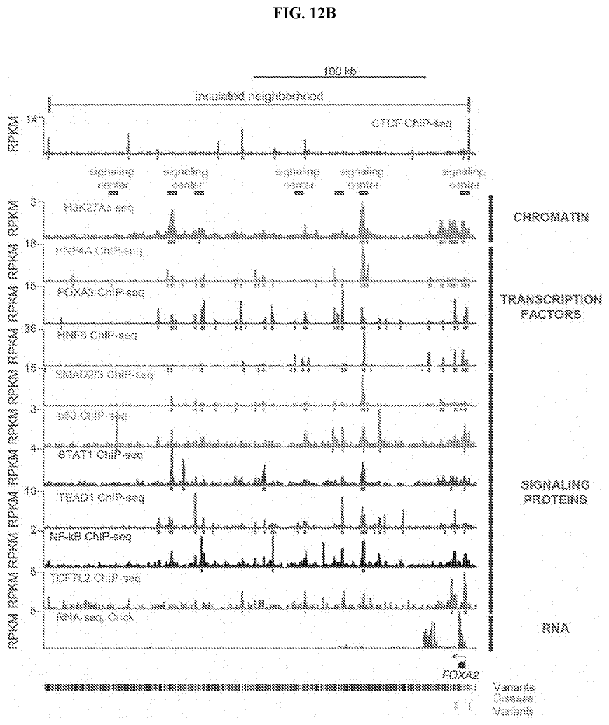

Table 33 shows that HMOX1, a gene implicated in liver fibrosis, was upregulated by BIO (MT-209), a Wnt signaling pathway agonist. FIG. 12A shows an insulated neighborhood containing HMOX1, MSM5, and RASD2 genes. The four GSCs in the IN are occupied cumulatively by transcription factors HNF4A, FOXA2, and ONECUT1, and signaling proteins GR, TCF7L2, SREBP1, SMAD2/3, TP53, STAT1, TEAD1, and NF-kB. Positions of the GSCs within the IN were determined partially based on presence of H3K27Ac chromatin modification. RNA-seq signal on the tracks reflects expression levels for the three genes. Known disease variants are shown below the RNA-seq tracks in FIG. 12A.

FIG. 12B shows an IN containing the FOXA2 gene. The seven GSCs in the IN are occupied cumulatively by transcription factors HNF4A, FOXA2, and HNF6 (ONECUT1), and genomic signaling proteins SMAD2/3, TP53, STAT1, TEAD1, NF-kB, and TCF7L2. Positions of the GSCs within the neighborhood were determined partially based on presence of H3K27Ac chromatin modification. RNA-seq signal on the tracks reflects expression levels for FOXA2. Known disease variants are shown below the RNA-seq tracks in FIG. 12B. Transcription factors HNF4A, FOXA2, and HNF6 (ONECUT1) are master transcription factors in human hepatocytes.

### Example 9. Predicting Expression Changes in Response to Compound

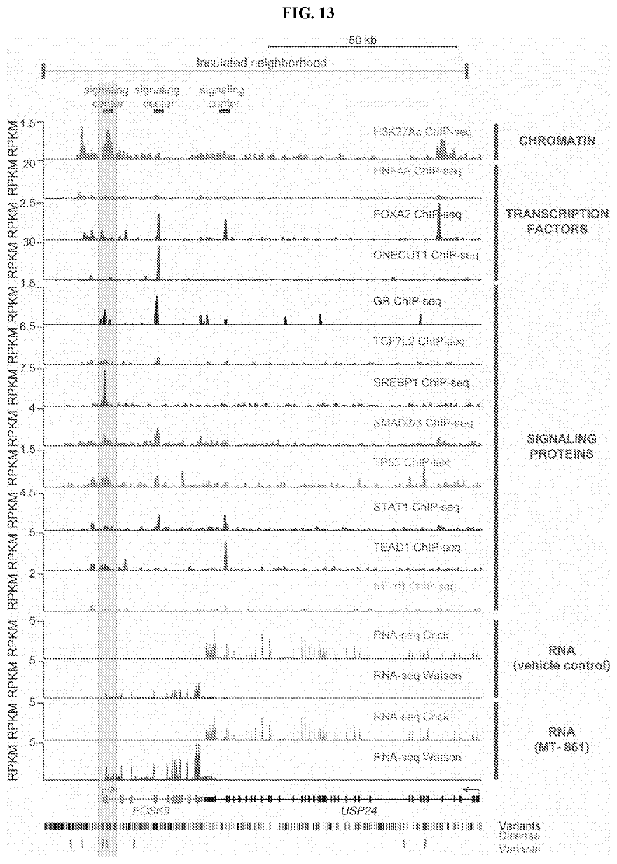

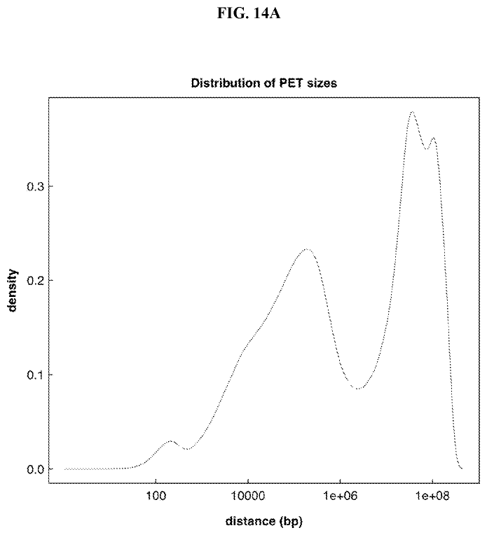

FIG. 13 shows the binding profile of the IN containing PCSK9 when samples were treated with prednisone (MT-861). SREBP1, associated with a sterol synthesis pathway, was observed to bind the GSC of PCSK9 and correspond to expression of PCSK9. PCSK9 was observed to be upregulated in Table 33 in response to simvastatin, which is a cholesterol biosynthesis signaling agonist. Therefore, the expression changes of PCSK9 in response to simvastatin may be predicted partially from the relationships between the ChIP-seq binding profile and the RNA-seq results in Table 33.

Assigning predictive values to the relationships among the experimental data herein depends on whether identifying the TF(s) or signaling protein(s) binding to a GSC is sufficient to predict changes in transcriptional output when the corresponding signaling pathway is perturbed. There is a signaling code, e.g., a set of rules beyond simply occupancy that determines transcriptional output.

### Example 10. Quality Control for ChIP-Seq

Quantitative metrics are used to evaluate genomic signaling center/gene expression correlations. Hypotheses to apply to positive controls include: whether GSCs contain target TF motifs, other TF motifs that are found in the GSCs, spatial relationship of TF occupancy, IN nesting, and combinations of SCs/motifs within IN. Temporal response, dose response, linkage weighting, and permutation complexity are also considered.

The results shown in the RNA-seq gene tracks were analyzed to determine whether the antibodies that were chosen to be specific to each of the ChIP-seq targets binds a region of the genome with a known binding motif. GSCs containing the associated motif were observed to be predictive for signaling. Table 42 provides metrics to measure the quality of the ChIP-seq results.

TABLE 42
Quality control (QC) for ChIP-seq targets

| | Peak | Most enriched | | Fraction of target | Motif | Quality |
|---|---|---|---|---|---|---|
| Target | number | motif | p-value | sequences w/Motif | rank | control |
| HNF4A | 88711 | HNF4A | 1E−6572 | 21.25% | 1 | Positive |
| FOXA2 | 73594 | FOXA2 | 1E−10960 | 45.02% | 1 | Positive |
| HNF1A | 13089 | HNF1A | 1E−738 | 7.86% | 1 | Positive |
| TCF7L2 | 8785 | TCF7L2 | 1E−1023 | 22.81% | 1 | Positive |
| NFκB-p65 | 42000 | NF-kB | 1E−2784 | 22.09% | 1 | Positive |
| SREBP1 | 180 | SREBP1 | 1E−18 | 19.13% | 1 | Positive |
| SMAD2/3 | 27571 | HNF4A | 1E−1305 | 16.22% | 1 | Positive |
| GRp20 | 5766 | CTCF | 1E−207 | 8.43% | 1 | Negative |
| ZGPAT | 36642 | FOXA1 | 1E−956 | 17.10% | 1 | Negative |
| CREB1 | 250035 | FOXA2 | 1E−850 | 20.53% | 1 | Negative |

### Example 11. Locating Occupancy-Dependent Signaling Centers (ODSCs) on Each Chromosome

Due to the diffuse binding patterns of signaling proteins, results were further limited to multi-footprint regions. ChIP-seq was performed to identify determine factors that may be used to identify an ODSC, such as a threshold number of signaling proteins, bound master transcription factors, and chromatin modifications. Master transcription factors for hepatocytes are defined in D'Alessio et al., Stem Cell Reports, Vol. 5; 763-775 (2015).

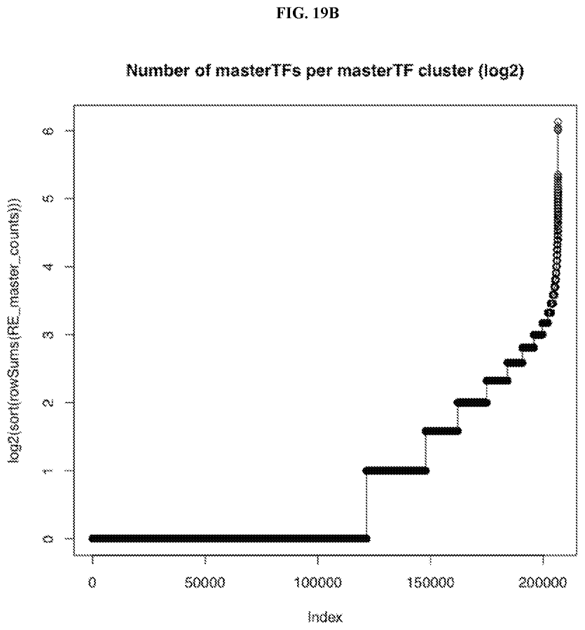

Signaling proteins and master transcription factors were grouped into dusters based on whether the called ChIP-seq peak overlap in linear genomic space. FIG. 19A shows a density plot of the log number of signaling protein binding events in each signaling protein cluster. Approximately half of clusters contain only one signaling protein binding event. FIG. 19B shows a density plot of the log number of master transcription factor binding event in each master transcription factor cluster in hepatocytes. More than half of all dusters was observed to contain only one master transcription factor binding event. From the results in FIG. 19A and FIG. 19B, a threshold value of at least 2 signaling proteins and at least 2 master transcription factors per each cluster was selected to define an occupancy dependent signaling center. ChIP-seq results for H3K27ac, Brd4, and p300 were combined with the results in FIG. 19A and FIG. 19B to further establish criteria for an ODSC.

Criteria were established that a region having at least one of H3K27ac or Brd4 or p300 or at least 2 master transcription factors and at least 2 signaling proteins ((H3K27ac or Brd4 or p300 or at least 2 master transcription factors)+at least 2 signaling proteins) is an ODSC. Using this definition, 38,656 ODSCs were identified. The locations of the ODSCs on Chromosome 1 are shown in Table 43. It is noted that the unique identifiers from ENSEMBL for the “Overlapping Gene(s)” have been modified to remove the first five leading zeros (0) of the identifier after the ENSG label (ENSG00000). The “Start” and “End” boundaries are in reference to nucleic acid positions in the hg19 genome of the UCSC Genome Browser Gateway.

TABLE 43
ODSC Locations on Chromosome 1

| SEQ ID NO. | Start | End | Overlapping Gene(s) |
|---|---|---|---|
| 32627 | 713857 | 714284 | 237491, 228327 |
| 32628 | 859081 | 859882 | |
| 32629 | 893725 | 894868 | 188976 |
| 32630 | 901414 | 902636 | 187583 |
| 32631 | 934902 | 937696 | 188290 |
| 32632 | 940118 | 942798 | |
| 32633 | 948209 | 949702 | 187608, 224969 |
| 32634 | 954620 | 956823 | 188157 |
| 32635 | 959883 | 960817 | 188157 |
| 32636 | 994398 | 995462 | 217801 |
| 32637 | 998954 | 1000393 | 217801 |
| 32638 | 1014613 | 1016031 | |
| 32639 | 1051393 | 1052182 | 131591 |
| 32640 | 1059974 | 1060984 | |
| 32641 | 1099803 | 1101365 | |
| 32642 | 1166019 | 1168675 | 176022, 078808 |
| 32643 | 1174938 | 1175933 | |
| 32644 | 1207524 | 1210311 | 160087 |
| 32645 | 1216361 | 1218351 | 162572 |
| 32646 | 1239959 | 1240566 | 131584 |
| 32647 | 1242087 | 1245155 | 169972, 131584 |
| 32648 | 1259153 | 1261364 | 224051, 127054 |
| 32649 | 1278643 | 1285594 | 107404 |
| 32650 | 1309507 | 1311757 | 175756 |
| 32651 | 1333596 | 1336815 | 224870, 221978 |
| 32652 | 1337276 | 1338146 | 242485, 224870 |
| 32653 | 1341543 | 1343821 | 242485 |
| 32654 | 1364978 | 1366597 | 225285 |
| 32655 | 1366604 | 1369554 | 225285 |
| 32656 | 1373362 | 1374320 | 179403 |
| 32657 | 1406328 | 1407232 | 160072 |
| 32658 | 1446485 | 1447630 | 197785 |
| 32659 | 1489904 | 1490911 | 160075 |
| 32660 | 1509070 | 1511629 | 215014, 160075 |
| 32661 | 1550584 | 1552276 | 197530, 272106 |
| 32662 | 1610112 | 1610664 | 189339 |
| 32663 | 1655573 | 1656167 | 227775, 008128, 268575 |
| 32664 | 1684695 | 1685510 | 008130 |
| 32665 | 1688757 | 1691274 | 008130 |
| 32666 | 1695074 | 1696041 | 008130 |
| 32667 | 1708474 | 1711165 | 008130 |
| 32668 | 1713795 | 1714807 | |
| 32669 | 1820929 | 1823640 | 231050, 078369 |
| 32670 | 1839462 | 1841569 | |
| 32671 | 1859400 | 1859915 | 142609 |
| 32672 | 1975926 | 1976713 | |
| 32673 | 1977878 | 1979421 | |
| 32674 | 1981106 | 1983066 | 067606, 226969 |
| 32675 | 2120804 | 2122810 | 269554, 162585 |
| 32676 | 2125554 | 2127163 | 162585 |
| 32677 | 2138288 | 2140518 | 162585 |
| 32678 | 2157992 | 2159433 | |
| 32679 | 2160223 | 2162241 | 157933 |
| 32680 | 2162469 | 2164678 | 157933 |
| 32681 | 2165082 | 2166440 | 157933 |
| 32682 | 2179076 | 2180445 | 157933 |
| 32683 | 2181472 | 2183525 | 157933 |
| 32684 | 2186944 | 2187790 | 157933 |
| 32685 | 2201505 | 2202588 | 157933 |
| 32686 | 2245704 | 2246958 | |
| 32687 | 2322267 | 2324538 | 157916, 116151 |
| 32688 | 2342905 | 2346387 | 157911 |
| 32689 | 2406408 | 2407027 | 149527 |
| 32690 | 2425699 | 2426060 | 229393, 149527 |
| 32691 | 2456557 | 2459220 | 157881 |
| 32692 | 2459508 | 2461364 | 197921 |
| 32693 | 2477557 | 2480991 | 272449 |
| 32694 | 2487017 | 2489200 | 157873, 238164 |
| 32695 | 2507231 | 2508062 | |
| 32696 | 2509578 | 2510609 | |
| 32697 | 2516493 | 2518599 | 157870 |
| 32698 | 2573477 | 2574898 | 215912 |
| 32699 | 2986219 | 2986762 | 142611 |
| 32700 | 3209528 | 3210155 | 142611 |
| 32701 | 3236643 | 3237647 | 142611 |
| 32702 | 3370576 | 3372262 | 130762 |
| 32703 | 3376396 | 3377249 | 130762 |
| 32704 | 3396394 | 3397583 | 130762 |
| 32705 | 3445753 | 3446391 | 162591 |
| 32706 | 3540401 | 3542579 | 158109, 238260 |
| 32707 | 3565899 | 3566958 | 116213 |
| 32708 | 3585220 | 3585777 | 227589, 078900 |
| 32709 | 3656537 | 3657040 | 227372 |
| 32710 | 3663146 | 3664121 | 227372 |
| 32711 | 3689174 | 3689791 | 235169 |
| 32712 | 3711928 | 3713827 | 130764 |
| 32713 | 3772894 | 3774861 | 169598, 116198 |
| 32714 | 3815920 | 3818154 | 236423, 198912 |
| 32715 | 4143549 | 4144569 | |
| 32716 | 4440619 | 4441092 | |
| 32717 | 5490837 | 5491374 | |
| 32718 | 5975830 | 5976457 | 131697 |
| 32719 | 5993639 | 5994358 | 131697 |
| 32720 | 6051538 | 6052630 | 131697, 069424 |
| 32721 | 6085988 | 6086471 | 069424 |
| 32722 | 6258636 | 6260608 | 116251 |
| 32723 | 6265111 | 6267058 | 158286, 226944, 116251 |
| 32724 | 6295039 | 6296332 | 225077, 116237 |
| 32725 | 6305086 | 6305721 | 173673 |
| 32726 | 6319432 | 6320436 | 158292 |
| 32727 | 6413250 | 6414827 | 097021 |
| 32728 | 6416318 | 6416806 | 097021 |
| 32729 | 6418789 | 6420993 | 097021 |
| 32730 | 6452636 | 6454818 | 271746, 097021 |
| 32731 | 6510907 | 6511710 | 187017 |
| 32732 | 6524255 | 6524926 | 215788 |
| 32733 | 6532074 | 6532809 | 171680 |
| 32734 | 6550203 | 6551906 | 171680 |
| 32735 | 6613708 | 6614984 | 162408 |
| 32736 | 6639121 | 6641206 | 204859, 173662 |
| 32737 | 6641999 | 6643107 | 204859 |
| 32738 | 6660162 | 6665440 | 162413 |
| 32739 | 6672646 | 6675958 | 116273, 162413 |
| 32740 | 6684927 | 6686117 | 041988 |
| 32741 | 6760651 | 6762692 | 007923 |
| 32742 | 6784559 | 6785120 | 228750 |
| 32743 | 6822095 | 6822990 | |
| 32744 | 6843552 | 6846644 | 171735, 237436 |
| 32745 | 6867631 | 6868607 | 171735 |
| 32746 | 6919307 | 6921013 | 171735 |
| 32747 | 7157903 | 7159085 | 171735 |
| 32748 | 7208581 | 7210426 | 171735 |
| 32749 | 7218993 | 7219774 | 171735 |
| 32750 | 7325087 | 7328694 | 171735 |
| 32751 | 7406455 | 7407752 | 171735 |
| 32752 | 7630241 | 7630855 | 171735 |
| 32753 | 7632711 | 7633509 | 171735 |
| 32754 | 7634118 | 7635164 | 171735 |
| 32755 | 7740204 | 7741216 | 171735 |
| 32756 | 7830913 | 7832475 | 049245 |
| 32757 | 7843743 | 7845272 | 049246 |
| 32758 | 8000619 | 8001301 | 049249 |
| 32759 | 8020585 | 8022813 | 116288 |
| 32760 | 8063335 | 8063885 | |
| 32761 | 8063915 | 8067250 | 116285 |
| 32762 | 8067874 | 8068391 | 116285 |
| 32763 | 8069705 | 8071594 | 116285 |
| 32764 | 8077372 | 8090318 | 238290, 116285 |
| 32765 | 8099140 | 8101568 | 238290 |
| 32766 | 8120825 | 8123294 | 238290 |
| 32767 | 8130301 | 8132101 | 238290 |
| 32768 | 8149435 | 8159312 | 238290 |
| 32769 | 8176000 | 8178054 | 238290 |
| 32770 | 8180456 | 8182472 | 238290 |
| 32771 | 8201116 | 8202191 | |
| 32772 | 8228962 | 8232922 | |
| 32773 | 8233833 | 8235332 | |
| 32774 | 8241522 | 8245276 | |
| 32775 | 8248458 | 8251154 | 229305 |
| 32776 | 8253640 | 8255849 | |
| 32777 | 8256053 | 8258915 | |
| 32778 | 8259998 | 8264186 | |
| 32779 | 8271233 | 8272850 | 227634 |
| 32780 | 8276508 | 8278363 | 233645 |
| 32781 | 8284669 | 8285851 | |
| 32782 | 8298106 | 8300290 | |
| 32783 | 8307103 | 8308643 | |
| 32784 | 8317066 | 8321901 | |
| 32785 | 8323050 | 8323689 | |
| 32786 | 8456020 | 8458074 | 142599 |
| 32787 | 8467998 | 8468691 | 142599 |
| 32788 | 8480118 | 8481361 | 142599 |
| 32789 | 8482342 | 8485254 | 232912, 142599 |
| 32790 | 8557458 | 8558186 | 142599 |
| 32791 | 8585054 | 8586425 | 142599 |
| 32792 | 8623558 | 8624829 | 142599 |
| 32793 | 8631260 | 8632640 | 142599 |
| 32794 | 8685692 | 8687291 | 142599 |
| 32795 | 8703267 | 8704607 | 142599 |
| 32796 | 8728136 | 8728701 | 142599 |
| 32797 | 8730330 | 8731182 | 142599 |
| 32798 | 8762105 | 8763757 | 142599 |
| 32799 | 8771984 | 8773420 | 142599 |
| 32800 | 8801981 | 8802615 | 142599 |
| 32801 | 8814962 | 8815494 | 142599 |
| 32802 | 8876418 | 8878755 | 142599 |
| 32803 | 8908688 | 8909356 | |
| 32804 | 8929733 | 8932388 | 074800 |
| 32805 | 8932818 | 8934618 | 074800 |
| 32806 | 8937076 | 8941437 | 230679, 074800, 236269 |
| 32807 | 8950027 | 8950658 | |
| 32808 | 8959839 | 8960606 | |
| 32809 | 8962777 | 8964460 | |
| 32810 | 9170654 | 9171274 | 180758 |
| 32811 | 9172422 | 9174108 | 180758 |
| 32812 | 9187639 | 9189801 | 180758 |
| 32813 | 9221802 | 9225824 | |
| 32814 | 9241243 | 9243591 | 234546, 228526 |
| 32815 | 9253872 | 9254924 | 228526 |
| 32816 | 9256288 | 9257339 | 228526 |
| 32817 | 9292849 | 9296301 | 049239 |
| 32818 | 9297627 | 9303507 | 049239 |
| 32819 | 9307965 | 9308820 | 049239 |
| 32820 | 9309209 | 9310926 | 049239 |
| 32821 | 9335562 | 9336657 | |
| 32822 | 9348952 | 9351363 | |
| 32823 | 9351400 | 9355734 | 171621 |
| 32824 | 9360014 | 9360804 | 171621 |
| 32825 | 9461435 | 9462756 | |
| 32826 | 9488312 | 9489871 | |
| 32827 | 9493900 | 9503319 | 252956 |
| 32828 | 9506472 | 9507947 | |
| 32829 | 9512550 | 9513121 | |
| 32830 | 9519859 | 9520529 | |
| 32831 | 9525115 | 9528127 | |
| 32832 | 9552002 | 9552788 | |
| 32833 | 9560878 | 9561969 | 235263 |
| 32834 | 9562807 | 9564048 | 235263 |
| 32835 | 9597781 | 9598720 | |
| 32836 | 9599088 | 9600628 | 171612 |
| 32837 | 9610103 | 9611162 | 171612 |
| 32838 | 9613593 | 9616588 | 171612 |
| 32839 | 9648436 | 9649981 | 188807 |
| 32840 | 9746741 | 9748896 | 231789, 171608 |
| 32841 | 9748973 | 9750336 | 171608 |
| 32842 | 9883263 | 9884131 | 171603 |
| 32843 | 9884898 | 9885957 | |
| 32844 | 9928117 | 9928711 | 178585 |
| 32845 | 9960451 | 9961038 | 178585 |
| 32846 | 9969389 | 9971505 | 178585 |
| 32847 | 10002279 | 10004326 | 228150, 173614, 162441 |
| 32848 | 10092253 | 10094085 | 130939 |
| 32849 | 10125150 | 10126008 | 130939 |
| 32850 | 10269529 | 10272664 | 054523 |
| 32851 | 10352078 | 10352665 | 054523 |
| 32852 | 10446959 | 10449280 | |
| 32853 | 10458455 | 10460868 | 142657 |
| 32854 | 10490112 | 10491351 | 251503, 175279, 271989 |
| 32855 | 10519084 | 10519785 | 203469, 160049 |
| 32856 | 10531506 | 10533000 | 142655, 160049 |
| 32857 | 10534619 | 10536169 | 142655 |
| 32858 | 10564262 | 10564830 | 142655 |
| 32859 | 10569235 | 10572405 | 142655 |
| 32860 | 10587512 | 10590811 | 142655 |
| 32861 | 10614249 | 10614731 | 142655 |
| 32862 | 10615563 | 10616648 | 142655 |
| 32863 | 10658281 | 10660554 | 142655 |
| 32864 | 10688626 | 10689378 | 142655 |
| 32865 | 10725888 | 10726401 | 130940 |
| 32866 | 10753406 | 10754713 | 130940 |
| 32867 | 10791105 | 10791415 | 130940 |
| 32868 | 10856096 | 10857015 | 130940 |
| 32869 | 10927032 | 10927798 | |
| 32870 | 11072145 | 11073609 | 120948 |
| 32871 | 11105644 | 11107700 | 009724 |
| 32872 | 11118713 | 11121250 | 116649 |
| 32873 | 11158844 | 11160668 | 230337, 171824 |
| 32874 | 11194179 | 11194843 | 198793 |
| 32875 | 11211705 | 11213642 | 253086, 198793 |
| 32876 | 11217265 | 11219652 | 198793 |
| 32877 | 11236295 | 11237129 | 198793 |
| 32878 | 11321625 | 11323626 | 198793 |
| 32879 | 11327210 | 11328430 | |
| 32880 | 11332701 | 11334539 | 120942 |
| 32881 | 11708453 | 11711096 | 116661 |
| 32882 | 11714220 | 11715445 | 132879, 116661 |
| 32883 | 11723935 | 11725355 | 116663 |
| 32884 | 11731201 | 11732070 | 116663 |
| 32885 | 11734855 | 11737322 | 116670 |
| 32886 | 11740476 | 11742013 | 116670 |
| 32887 | 11795901 | 11798581 | 177674 |
| 32888 | 11837451 | 11838376 | 177553, 215910 |
| 32889 | 11860769 | 11861251 | 177000 |
| 32890 | 11864394 | 11867634 | 011021, 177000 |
| 32891 | 11899536 | 11901111 | 242349, 011021 |
| 32892 | 11902102 | 11903251 | 011021, 242349 |
| 32893 | 11967495 | 11970502 | 201801, 199347 |
| 32894 | 11984452 | 11985361 | 116685 |
| 32895 | 11987826 | 11988820 | |
| 32896 | 11993950 | 11995476 | 083444 |
| 32897 | 12039320 | 12041545 | 116688 |
| 32898 | 12049224 | 12053091 | 116688 |
| 32899 | 12079100 | 12080535 | 116691 |
| 32900 | 12190466 | 12191316 | 120949 |
| 32901 | 12206678 | 12207767 | |
| 32902 | 12208096 | 12208919 | |
| 32903 | 12219675 | 12220453 | |
| 32904 | 12221327 | 12222281 | |
| 32905 | 12226710 | 12228198 | 028137 |
| 32906 | 12230116 | 12230760 | 028137 |
| 32907 | 12240230 | 12241339 | 028137 |
| 32908 | 12244067 | 12247040 | 028137 |
| 32909 | 12266564 | 12267768 | 028137 |
| 32910 | 12280834 | 12281554 | 225196, 252969 |
| 32911 | 12289729 | 12291849 | 048707 |
| 32912 | 12406417 | 12407486 | 048707 |
| 32913 | 12415258 | 12419170 | 048707 |
| 32914 | 12446195 | 12446796 | 048707 |
| 32915 | 12465338 | 12466172 | 048707 |
| 32916 | 12492909 | 12494639 | 048707 |
| 32917 | 12497006 | 12498957 | 048707 |
| 32918 | 12513098 | 12514840 | 048707 |
| 32919 | 12537705 | 12538907 | 048707 |
| 32920 | 12584815 | 12586642 | |
| 32921 | 12587208 | 12589842 | 229484 |
| 32922 | 12609670 | 12610292 | |
| 32923 | 12650986 | 12652358 | 162496 |
| 32924 | 12653593 | 12658285 | 162496 |
| 32925 | 12660261 | 12665352 | 162496 |
| 32926 | 12667960 | 12670336 | 162496 |
| 32927 | 12673974 | 12680593 | 272482, 162496 |
| 32928 | 13977734 | 13978281 | |
| 32929 | 14074906 | 14076929 | 116731 |
| 32930 | 14084013 | 14084520 | 116731 |
| 32931 | 14125327 | 14125734 | 116731 |
| 32932 | 14125963 | 14126500 | 116731 |
| 32933 | 15478765 | 15479423 | 171729, 175147 |
| 32934 | 15528381 | 15528988 | 171729 |
| 32935 | 15678669 | 15680002 | 142621 |
| 32936 | 15692730 | 15693489 | 142621 |
| 32937 | 15735742 | 15737587 | 142634, 228140 |
| 32938 | 15747496 | 15748460 | 142634 |
| 32939 | 15850024 | 15852020 | 132906 |
| 32940 | 15852724 | 15854096 | 116138, 132906 |
| 32941 | 15885642 | 15886461 | 116138 |
| 32942 | 15901287 | 15901774 | 116771, 116138 |
| 32943 | 15910558 | 15912163 | 116771, 116138 |
| 32944 | 15929674 | 15930760 | 237301 |
| 32945 | 15942981 | 15945997 | 197312 |
| 32946 | 16009315 | 16011944 | 271742, 264048, 116786 |
| 32947 | 16044763 | 16045426 | 116786 |
| 32948 | 16061720 | 16063736 | 162461, 237938 |
| 32949 | 16068629 | 16069679 | 162460, 224459 |
| 32950 | 16105950 | 16108296 | 162458 |
| 32951 | 16121485 | 16125021 | |
| 32952 | 16125125 | 16128137 | |
| 32953 | 16158212 | 16164396 | 179743 |
| 32954 | 16173248 | 16177312 | 065526, 179743 |
| 32955 | 16211610 | 16213389 | 065526 |
| 32956 | 16301749 | 16302926 | 116809 |
| 32957 | 16309118 | 16309836 | |
| 32958 | 16338751 | 16340132 | |
| 32959 | 16393147 | 16394262 | 185519 |
| 32960 | 16439349 | 16440036 | |
| 32961 | 16469783 | 16474039 | 142627 |
| 32962 | 16479943 | 16483681 | 227959, 142627 |
| 32963 | 16490616 | 16492011 | |
| 32964 | 16501674 | 16503536 | |
| 32965 | 16504009 | 16506220 | |
| 32966 | 16507294 | 16511947 | |
| 32967 | 16553183 | 16553827 | 237276 |
| 32968 | 16574413 | 16575703 | 037637 |
| 32969 | 16579824 | 16581964 | 037637 |
| 32970 | 16597285 | 16597868 | 037637 |
| 32971 | 16676059 | 16676940 | 037637 |
| 32972 | 16677183 | 16679788 | 055070, 037637 |
| 32973 | 16693036 | 16695098 | 055070 |
| 32974 | 16766827 | 16768149 | 157191 |
| 32975 | 16839993 | 16841033 | 206652 |
| 32976 | 16921681 | 16922179 | 219481 |
| 32977 | 16934316 | 16935754 | 219481 |
| 32978 | 16938943 | 16940490 | 219481 |
| 32979 | 16970438 | 16971442 | 215908 |
| 32980 | 17023367 | 17024335 | 268869 |
| 32981 | 17035156 | 17035897 | 268869 |
| 32982 | 17215253 | 17216535 | 235241, 238142, 058453 |
| 32983 | 17222412 | 17223908 | 207005, 058453 |
| 32984 | 17230140 | 17232387 | 272426, 058453 |
| 32985 | 17240350 | 17241303 | 058453 |
| 32986 | 17287025 | 17287735 | 058453 |
| 32987 | 17337490 | 17338632 | 159363 |
| 32988 | 17379588 | 17381086 | 117118 |
| 32989 | 17389891 | 17390794 | |
| 32990 | 17407866 | 17408345 | 117115 |
| 32991 | 17424590 | 17425480 | 117115 |
| 32992 | 17559353 | 17560895 | 142623 |
| 32993 | 17563328 | 17564572 | 142623 |
| 32994 | 17763652 | 17767407 | 179051 |
| 32995 | 17767420 | 17768428 | |
| 32996 | 17842924 | 17843864 | |
| 32997 | 17853487 | 17854510 | |
| 32998 | 17864412 | 17865127 | |
| 32999 | 17865157 | 17867452 | 074964 |
| 33000 | 17878018 | 17879138 | 074964 |
| 33001 | 17884907 | 17887194 | 074964 |
| 33002 | 17887236 | 17888504 | 074964 |
| 33003 | 17890417 | 17891723 | 074964 |
| 33004 | 17895449 | 17897391 | 074964 |
| 33005 | 17902147 | 17903200 | 074964 |
| 33006 | 17905984 | 17910546 | 074964 |
| 33007 | 17911583 | 17913143 | 074964 |
| 33008 | 17913757 | 17915419 | 074964 |
| 33009 | 17918081 | 17921040 | 074964 |
| 33010 | 17950626 | 17951894 | 074964 |
| 33011 | 17958857 | 17960073 | 074964 |
| 33012 | 17960147 | 17960696 | 074964 |
| 33013 | 18320340 | 18320965 | |
| 33014 | 18454461 | 18455585 | 117154 |
| 33015 | 18509027 | 18510277 | 117154 |
| 33016 | 18790674 | 18791608 | |
| 33017 | 18805872 | 18808372 | 179023 |
| 33018 | 19208866 | 19209387 | 255275, 159423 |
| 33019 | 19212538 | 19213176 | 255275, 159423 |
| 33020 | 19217094 | 19218106 | 255275, 159423 |
| 33021 | 19221039 | 19222181 | 255275, 159423 |
| 33022 | 19225887 | 19229978 | 159423, 255275 |
| 33023 | 19248509 | 19249350 | 169991 |
| 33024 | 19274929 | 19276643 | 169991 |
| 33025 | 19278156 | 19278858 | 169991 |
| 33026 | 19280153 | 19280562 | 169991 |
| 33027 | 19281487 | 19283398 | 169991 |
| 33028 | 19335875 | 19339458 | |
| 33029 | 19339968 | 19340999 | |
| 33030 | 19355739 | 19356302 | |
| 33031 | 19371653 | 19373248 | |
| 33032 | 19391929 | 19395488 | |
| 33033 | 19405827 | 19408172 | 127481 |
| 33034 | 19412436 | 19414948 | 127481 |
| 33035 | 19528657 | 19529341 | 127481 |
| 33036 | 19535294 | 19537871 | 230424, 127481 |
| 33037 | 19577073 | 19579111 | 053372, 127463 |
| 33038 | 19597956 | 19601577 | 211454 |
| 33039 | 19613744 | 19615980 | 162482 |
| 33040 | 19637770 | 19639918 | 040487, 053371 |
| 33041 | 19664568 | 19665536 | 077549 |
| 33042 | 19751865 | 19752716 | 077549 |
| 33043 | 19756677 | 19757139 | 077549 |
| 33044 | 19763139 | 19763750 | 077549 |
| 33045 | 19766926 | 19767296 | 077549 |
| 33046 | 19772307 | 19772907 | 077549 |
| 33047 | 19777171 | 19777700 | 077549 |
| 33048 | 19810501 | 19813484 | 077549 |
| 33049 | 19831280 | 19831850 | |
| 33050 | 19922515 | 19924980 | 270136, 173436, 235185 |
| 33051 | 19949768 | 19950591 | 270136, 173436 |
| 33052 | 20061577 | 20063588 | 162542 |
| 33053 | 20080717 | 20081395 | 162542 |
| 33054 | 20125457 | 20127390 | 162542 |
| 33055 | 20141462 | 20143161 | 178828, 235434 |
| 33056 | 20208516 | 20209761 | 169914 |
| 33057 | 20314090 | 20314633 | |
| 33058 | 20478284 | 20481908 | 227066 |
| 33059 | 20509514 | 20511052 | 225986 |
| 33060 | 20512002 | 20513307 | 162543, 225986 |
| 33061 | 20537916 | 20538411 | |
| 33062 | 20539174 | 20539883 | |
| 33063 | 20802338 | 20803780 | |
| 33064 | 20803839 | 20805139 | |
| 33065 | 20806081 | 20808696 | |
| 33066 | 20810115 | 20813903 | 162545 |
| 33067 | 20833286 | 20835486 | 090432 |
| 33068 | 20913425 | 20917814 | 158825 |
| 33069 | 20927163 | 20932186 | 158825 |
| 33070 | 20939056 | 20941609 | 158825 |
| 33071 | 20959273 | 20961711 | 158828 |
| 33072 | 20987036 | 20988931 | 244038 |
| 33073 | 21022669 | 21024027 | 117245 |
| 33074 | 21057818 | 21061162 | 233069, 189410 |
| 33075 | 21064337 | 21066823 | |
| 33076 | 21112011 | 21114971 | 127483 |
| 33077 | 21140142 | 21141078 | 075151 |
| 33078 | 21173886 | 21174640 | 075151 |
| 33079 | 21223041 | 21224059 | 075151 |
| 33080 | 21347891 | 21349987 | 075151 |
| 33081 | 21385472 | 21386180 | 075151 |
| 33082 | 21441240 | 21442261 | 075151 |
| 33083 | 21449761 | 21450459 | 075151 |
| 33084 | 21500925 | 21504341 | 266867, 075151 |
| 33085 | 21538546 | 21539041 | |
| 33086 | 21578291 | 21579607 | 117298 |
| 33087 | 21587477 | 21590075 | 117298 |
| 33088 | 21594709 | 21595752 | 117298 |
| 33089 | 21616082 | 21617514 | 117298 |
| 33090 | 21625387 | 21626418 | 231105, 117298 |
| 33091 | 21650351 | 21653173 | 117298 |
| 33092 | 21655419 | 21656836 | 117298 |
| 33093 | 21663928 | 21666321 | 117298 |
| 33094 | 21670639 | 21672925 | 117298 |
| 33095 | 21864052 | 21865113 | 162551 |
| 33096 | 21872198 | 21873506 | 162551 |
| 33097 | 21876406 | 21878561 | 162551 |
| 33098 | 21913268 | 21913887 | 233431 |
| 33099 | 21948136 | 21950675 | 076864 |
| 33100 | 21964951 | 21966715 | 076864 |
| 33101 | 21995251 | 21996699 | 076864 |
| 33102 | 22091057 | 22091855 | 090686 |
| 33103 | 22108320 | 22110620 | 090686 |
| 33104 | 22263629 | 22264521 | 142798 |
| 33105 | 22265613 | 22266691 | |
| 33106 | 22351090 | 22352884 | 218510, 228397 |
| 33107 | 22374887 | 22375329 | |
| 33108 | 22378871 | 22380741 | 070831 |
| 33109 | 22453833 | 22454182 | 162552 |
| 33110 | 22555716 | 22556444 | |
| 33111 | 22586012 | 22586868 | |
| 33112 | 22665576 | 22666282 | |
| 33113 | 22674596 | 22675399 | |
| 33114 | 22739573 | 22741670 | |
| 33115 | 22773528 | 22774571 | |
| 33116 | 22777582 | 22779291 | 184677 |
| 33117 | 22799124 | 22799577 | 184677 |
| 33118 | 22829695 | 22830966 | 184677 |
| 33119 | 23036990 | 23037378 | 133216 |
| 33120 | 23071976 | 23074349 | 133216 |
| 33121 | 23075761 | 23078489 | 133216 |
| 33122 | 23302348 | 23303710 | |
| 33123 | 23344942 | 23347124 | 240553, 004487 |
| 33124 | 23425730 | 23429824 | 169641 |
| 33125 | 23475500 | 23476167 | 169641 |
| 33126 | 23493695 | 23495570 | 169641 |
| 33127 | 23503601 | 23504801 | 169641 |
| 33128 | 23514033 | 23514648 | |
| 33129 | 23608825 | 23611147 | 261326 |
| 33130 | 23669080 | 23672170 | 125944 |
| 33131 | 23693776 | 23696292 | 249087, 125945 |
| 33132 | 23703397 | 23704380 | |
| 33133 | 23730222 | 23730977 | 204219 |
| 33134 | 23749891 | 23751935 | 204219 |
| 33135 | 23770411 | 23771292 | 088280 |
| 33136 | 23801248 | 23802086 | 088280 |
| 33137 | 23805019 | 23806606 | 088280 |
| 33138 | 23809624 | 23811278 | 088280 |
| 33139 | 23866184 | 23866790 | |
| 33140 | 23880590 | 23883579 | |
| 33141 | 23885723 | 23886497 | 117318 |
| 33142 | 23888677 | 23889084 | |
| 33143 | 23902982 | 23904583 | |
| 33144 | 23999584 | 24000640 | 229239 |
| 33145 | 24017907 | 24019603 | 142676 |
| 33146 | 24047009 | 24047615 | |
| 33147 | 24053389 | 24054469 | |
| 33148 | 24054950 | 24055905 | |
| 33149 | 24058688 | 24059365 | |
| 33150 | 24061378 | 24062204 | |
| 33151 | 24069139 | 24071843 | 011007 |
| 33152 | 24104435 | 24105961 | 057757, 236810 |
| 33153 | 24116729 | 24118702 | 011009 |
| 33154 | 24125233 | 24128666 | 117305, 117308 |
| 33155 | 24136116 | 24136818 | 117305 |
| 33156 | 24150987 | 24152338 | 117305 |
| 33157 | 24165035 | 24165778 | 117305 |
| 33158 | 24193988 | 24195532 | 179163 |
| 33159 | 24246403 | 24247648 | 188822 |
| 33160 | 24277903 | 24278625 | 188822 |
| 33161 | 24285004 | 24287510 | 189266, 188822 |
| 33162 | 24305904 | 24307619 | 188529 |
| 33163 | 24345147 | 24345845 | |
| 33164 | 24373595 | 24374089 | 225315 |
| 33165 | 24446223 | 24447271 | 142677 |
| 33166 | 24454109 | 24454819 | 142677 |
| 33167 | 24469077 | 24469998 | 142677 |
| 33168 | 24476365 | 24477993 | |
| 33169 | 24481383 | 24482099 | 185436 |
| 33170 | 24513197 | 24514561 | 185436 |
| 33171 | 24562430 | 24564250 | |
| 33172 | 24619940 | 24622465 | |
| 33173 | 24647767 | 24649477 | 232298, 158055 |
| 33174 | 24652537 | 24652990 | 158055 |
| 33175 | 24717545 | 24719306 | 001460 |
| 33176 | 24739542 | 24740725 | 001460 |
| 33177 | 24741558 | 24743783 | 001461, 001460 |
| 33178 | 24828336 | 24830042 | 117602 |
| 33179 | 24866313 | 24868748 | 117602, 264443 |
| 33180 | 24881804 | 24883031 | 184454, 264443 |
| 33181 | 24893815 | 24895518 | 184454 |
| 33182 | 24967752 | 24970881 | 133226 |
| 33183 | 25031649 | 25032613 | |
| 33184 | 25044062 | 25045932 | |
| 33185 | 25062187 | 25063305 | |
| 33186 | 25071202 | 25073790 | 169504 |
| 33187 | 25093669 | 25094364 | 169504 |
| 33188 | 25095529 | 25097843 | 169504 |
| 33189 | 25106206 | 25107509 | 169504 |
| 33190 | 25110197 | 25110919 | 169504 |
| 33191 | 25112477 | 25113118 | 169504 |
| 33192 | 25174321 | 25175634 | |
| 33193 | 25205225 | 25205660 | |
| 33194 | 25494416 | 25495563 | |
| 33195 | 25557951 | 25559976 | 117614 |
| 33196 | 25565653 | 25567487 | |
| 33197 | 25572325 | 25574912 | 272432, 117616 |
| 33198 | 25664697 | 25666232 | 117616, 183726 |
| 33199 | 25751462 | 25753033 | 188672 |
| 33200 | 25755874 | 25758829 | 204178, 188672 |
| 33201 | 25785113 | 25785979 | 204178 |
| 33202 | 25869715 | 25871298 | 157978 |
| 33203 | 25897924 | 25898719 | |
| 33204 | 25899375 | 25900873 | |
| 33205 | 25927252 | 25927990 | |
| 33206 | 25942529 | 25943849 | |
| 33207 | 25998278 | 25999097 | 117643 |
| 33208 | 26008264 | 26009119 | 117643 |
| 33209 | 26021219 | 26022662 | 117643 |
| 33210 | 26099763 | 26100949 | 117643 |
| 33211 | 26125941 | 26127630 | 162430 |
| 33212 | 26145745 | 26148030 | 223474, 228172, 255054, |
| | | | 117640 |
| 33213 | 26193652 | 26194448 | 182749 |
| 33214 | 26199475 | 26200055 | |
| 33215 | 26201361 | 26202759 | |
| 33216 | 26216034 | 26217528 | 117632 |
| 33217 | 26231248 | 26232388 | 117632 |
| 33218 | 26232448 | 26233479 | 264021, 117632 |
| 33219 | 26249409 | 26250384 | |
| 33220 | 26323114 | 26325364 | 158006 |
| 33221 | 26361832 | 26363874 | 158014, 158008 |
| 33222 | 26398631 | 26399442 | |
| 33223 | 26401717 | 26403212 | |
| 33224 | 26406432 | 26407211 | |
| 33225 | 26437038 | 26439046 | 175087 |
| 33226 | 26440421 | 26441667 | 175087 |
| 33227 | 26495914 | 26497428 | 142684, 236782 |
| 33228 | 26551433 | 26552693 | 236155 |
| 33229 | 26559950 | 26561856 | 130695 |
| 33230 | 26602286 | 26607691 | 142669, 130695 |
| 33231 | 26629227 | 26629843 | 158062 |
| 33232 | 26657449 | 26658866 | 176092 |
| 33233 | 26662811 | 26663693 | 176092 |
| 33234 | 26676513 | 26679089 | 176092 |
| 33235 | 26685035 | 26686206 | |
| 33236 | 26734992 | 26735489 | |
| 33237 | 26758054 | 26759722 | 117682 |
| 33238 | 26798641 | 26800271 | 198830 |
| 33239 | 26826312 | 26828388 | |
| 33240 | 26855622 | 26858144 | 117676 |
| 33241 | 26945555 | 26948843 | |
| 33242 | 27014272 | 27014691 | |
| 33243 | 27018449 | 27025554 | 260063, 117713 |
| 33244 | 27031924 | 27033797 | 117713 |
| 33245 | 27049896 | 27050597 | 117713 |
| 33246 | 27066350 | 27067176 | 117713 |
| 33247 | 27097400 | 27100380 | 117713 |
| 33248 | 27110369 | 27111102 | |
| 33249 | 27112214 | 27116338 | 060642 |
| 33250 | 27125199 | 27128125 | |
| 33251 | 27135913 | 27137095 | |
| 33252 | 27152371 | 27154420 | 204160 |
| 33253 | 27158059 | 27158734 | 204160 |
| 33254 | 27174236 | 27177929 | 204160 |
| 33255 | 27178573 | 27181019 | 204160 |
| 33256 | 27189638 | 27193077 | 175793 |
| 33257 | 27215601 | 27217632 | 198746, 142751 |
| 33258 | 27225428 | 27227750 | 090273, 198746 |
| 33259 | 27236727 | 27242639 | 131910, 090273 |
| 33260 | 27247709 | 27250478 | 090273 |
| 33261 | 27264319 | 27265544 | 090273 |
| 33262 | 27286136 | 27287131 | 175707 |
| 33263 | 27316915 | 27318754 | |
| 33264 | 27319319 | 27321532 | 253368 |
| 33265 | 27323715 | 27327709 | 253368 |
| 33266 | 27328140 | 27328979 | |
| 33267 | 27329243 | 27329725 | |
| 33268 | 27437258 | 27439278 | 090020 |
| 33269 | 27449093 | 27449628 | 090020 |
| 33270 | 27480362 | 27482703 | 090020 |
| 33271 | 27517864 | 27519663 | |
| 33272 | 27560702 | 27562313 | 142784, 224311 |
| 33273 | 27648350 | 27649621 | 186501 |
| 33274 | 27691909 | 27693652 | 142733 |
| 33275 | 27814959 | 27817607 | 158195 |
| 33276 | 27845717 | 27846840 | |
| 33277 | 27868191 | 27870286 | 126705 |
| 33278 | 27871661 | 27872939 | 126705 |
| 33279 | 27875525 | 27876510 | 126705 |
| 33280 | 27889615 | 27891813 | 126705 |
| 33281 | 27901733 | 27902830 | 126705 |
| 33282 | 27929370 | 27930987 | 126705 |
| 33283 | 27934973 | 27936004 | |
| 33284 | 27995694 | 27996348 | 225886, 126709 |
| 33285 | 27998489 | 27999545 | 126709 |
| 33286 | 28051828 | 28053337 | 009780 |
| 33287 | 28098440 | 28100748 | 269971, 117758 |
| 33288 | 28143467 | 28144278 | 117758 |
| 33289 | 28156626 | 28158523 | 117751 |
| 33290 | 28240054 | 28241688 | 117748 |
| 33291 | 28278593 | 28279085 | 227050, 130768 |
| 33292 | 28285869 | 28287138 | 158156, 227050 |
| 33293 | 28413967 | 28415602 | 158161 |
| 33294 | 28481297 | 28481812 | 169403 |
| 33295 | 28558672 | 28559756 | 126698 |
| 33296 | 28561886 | 28563980 | 130770 |
| 33297 | 28572846 | 28574141 | 271398, 130770 |
| 33298 | 28574400 | 28577249 | |
| 33299 | 28584578 | 28588391 | 130766 |
| 33300 | 28608613 | 28610042 | 130766 |
| 33301 | 28616981 | 28618034 | |
| 33302 | 28622296 | 28624265 | |
| 33303 | 28639612 | 28641022 | |
| 33304 | 28654705 | 28656748 | 130772 |
| 33305 | 28668015 | 28668655 | |
| 33306 | 28669795 | 28670305 | |
| 33307 | 28680060 | 28681610 | |
| 33308 | 28695394 | 28697309 | 204138 |
| 33309 | 28763777 | 28764970 | 204138 |
| 33310 | 28831518 | 28838009 | 200087, 242125, 180198 |
| 33311 | 28838633 | 28839460 | 180198 |
| 33312 | 28843975 | 28845724 | 238821, 180198 |
| 33313 | 28878908 | 28880566 | 180098 |
| 33314 | 28907436 | 28908858 | 197989 |
| 33315 | 28968602 | 28970650 | 229388, 120656 |
| 33316 | 28974205 | 28975997 | 270103, 229388 |
| 33317 | 28994511 | 28996716 | 162419 |
| 33318 | 29062332 | 29064822 | 198492 |
| 33319 | 29210734 | 29215180 | 159023 |
| 33320 | 29240759 | 29241443 | 159023 |
| 33321 | 29292605 | 29293774 | 159023 |
| 33322 | 29297788 | 29301366 | 159023 |
| 33323 | 29309895 | 29311195 | 159023 |
| 33324 | 29432390 | 29433085 | 159023 |
| 33325 | 29447782 | 29448896 | 253304 |
| 33326 | 29448920 | 29449654 | 253304 |
| 33327 | 29449777 | 29451658 | 253304 |
| 33328 | 29471610 | 29473782 | 225750 |
| 33329 | 29492680 | 29493233 | 116350 |
| 33330 | 29506780 | 29509660 | 116350 |
| 33331 | 29556599 | 29558183 | 116353 |
| 33332 | 29562660 | 29565306 | 060656 |
| 33333 | 29584906 | 29586712 | 060656 |
| 33334 | 30041889 | 30044380 | |
| 33335 | 30445163 | 30448318 | |
| 33336 | 30457217 | 30458323 | |
| 33337 | 30481566 | 30482621 | |
| 33338 | 30994218 | 30997194 | |
| 33339 | 30998090 | 31000060 | |
| 33340 | 31191353 | 31192706 | 186056, 162510 |
| 33341 | 31217966 | 31219924 | 162511 |
| 33342 | 31294402 | 31295464 | |
| 33343 | 31296149 | 31297579 | 223382 |
| 33344 | 31439517 | 31440077 | 134644 |
| 33345 | 31537003 | 31539079 | 134644 |
| 33346 | 31584065 | 31584570 | |
| 33347 | 31627363 | 31628755 | |
| 33348 | 31635456 | 31636021 | |
| 33349 | 31643371 | 31644208 | |
| 33350 | 31669420 | 31669951 | 084628 |
| 33351 | 31768799 | 31770589 | 121766, 060688 |
| 33352 | 31841244 | 31842156 | 121769 |
| 33353 | 31885641 | 31892495 | 168528 |
| 33354 | 31894093 | 31896220 | 168528 |
| 33355 | 31916613 | 31919025 | |
| 33356 | 31921833 | 31923233 | |
| 33357 | 31943183 | 31945623 | |
| 33358 | 31956106 | 31956704 | |
| 33359 | 31974283 | 31975668 | |
| 33360 | 31976671 | 31978259 | |
| 33361 | 31987724 | 31990619 | 223907 |
| 33362 | 32013774 | 32015945 | |
| 33363 | 32039378 | 32040136 | 229167 |
| 33364 | 32040911 | 32042049 | 229167 |
| 33365 | 32053246 | 32053878 | 142910 |
| 33366 | 32109286 | 32111520 | 264078, 235790, 162517 |
| 33367 | 32166147 | 32167475 | 084636 |
| 33368 | 32169966 | 32171119 | |
| 33369 | 32376523 | 32377109 | 269967, 184007 |
| 33370 | 32402201 | 32406152 | 184007 |
| 33371 | 32419623 | 32422496 | |
| 33372 | 32478747 | 32480737 | 121774 |
| 33373 | 32537399 | 32539207 | 121775, 203325 |
| 33374 | 32559776 | 32561772 | 121775 |
| 33375 | 32569748 | 32570171 | |
| 33376 | 32573174 | 32575075 | 025800 |
| 33377 | 32599060 | 32599517 | 025800 |
| 33378 | 32624799 | 32625180 | 025800 |
| 33379 | 32643657 | 32646642 | 084652 |
| 33380 | 32687049 | 32689128 | 084623, 160055 |
| 33381 | 32705884 | 32707410 | 220785 |
| 33382 | 32756756 | 32758696 | 116478 |
| 33383 | 32786691 | 32788817 | 116478 |
| 33384 | 32816471 | 32818263 | 162526 |
| 33385 | 32827119 | 32828239 | 162526, 225828 |
| 33386 | 32858694 | 32861303 | 160058 |
| 33387 | 32871302 | 32873883 | |
| 33388 | 32965643 | 32966835 | 254553 |
| 33389 | 33004755 | 33005739 | 160062, 254553 |
| 33390 | 33115320 | 33117935 | 162521, 176261 |
| 33391 | 33177710 | 33178296 | |
| 33392 | 33182073 | 33182689 | |
| 33393 | 33202206 | 33203664 | |
| 33394 | 33206477 | 33209157 | 162522 |
| 33395 | 33223923 | 33228279 | 162522 |
| 33396 | 33254103 | 33255379 | 134684 |
| 33397 | 33279644 | 33280379 | 134684 |
| 33398 | 33281587 | 33284301 | 116497, 134684 |
| 33399 | 33329874 | 33330690 | 160097 |
| 33400 | 33366325 | 33367759 | 121900 |
| 33401 | 33377777 | 33378934 | |
| 33402 | 33385432 | 33387141 | |
| 33403 | 33398992 | 33400196 | |
| 33404 | 33401231 | 33403101 | 116514 |
| 33405 | 33413851 | 33414957 | 116514 |
| 33406 | 33429199 | 33431029 | 116514 |
| 33407 | 33446392 | 33447622 | |
| 33408 | 33454341 | 33455282 | 236065 |
| 33409 | 33462173 | 33464002 | 236065 |
| 33410 | 33476606 | 33477231 | 004455, 236065 |
| 33411 | 33487287 | 33488949 | 004455, 236065 |
| 33412 | 33492728 | 33493130 | 004455, 236065 |
| 33413 | 33501587 | 33503040 | 004455 |
| 33414 | 33513467 | 33514211 | 004455 |
| 33415 | 33520649 | 33521857 | 004455 |
| 33416 | 33546566 | 33547484 | 142920, 004455 |
| 33417 | 33591843 | 33594044 | |
| 33418 | 33722063 | 33724863 | 160094 |
| 33419 | 33761023 | 33762920 | 160094 |
| 33420 | 33803536 | 33809027 | 134686 |
| 33421 | 33810558 | 33816566 | 233246, 134686 |
| 33422 | 33895724 | 33896825 | 134686 |
| 33423 | 33899300 | 33899751 | |
| 33424 | 33938048 | 33938981 | 121903 |
| 33425 | 34367963 | 34370018 | 121904 |
| 33426 | 34664711 | 34666607 | 142698 |
| 33427 | 35228277 | 35229713 | 189433, 163866, 255811 |
| 33428 | 35246533 | 35247547 | 188910, 163866, 255811 |
| 33429 | 35290847 | 35291692 | 163866 |
| 33430 | 35317156 | 35317711 | 230163, 163866 |
| 33431 | 35324208 | 35326260 | 163866 |
| 33432 | 35330860 | 35332796 | 116544 |
| 33433 | 35449583 | 35451613 | 243749, 241014, 163867, |
| | | | 271741 |
| 33434 | 35544328 | 35545924 | 197056 |
| 33435 | 35627618 | 35628527 | |
| 33436 | 35644100 | 35645835 | 116560 |
| 33437 | 35657260 | 35659142 | 116560 |
| 33438 | 35733759 | 35735395 | 146463 |
| 33439 | 35810907 | 35812086 | 146463 |
| 33440 | 35820284 | 35820783 | 146463 |
| 33441 | 35913301 | 35914512 | 142687 |
| 33442 | 35919393 | 35920969 | 142687 |
| 33443 | 36020840 | 36024292 | 020129, 142687 |
| 33444 | 36102624 | 36104180 | 126067 |
| 33445 | 36105810 | 36108426 | 126067 |
| 33446 | 36183630 | 36185346 | 142686 |
| 33447 | 36234747 | 36235212 | 092853 |
| 33448 | 36273200 | 36275399 | 134698 |
| 33449 | 36348575 | 36349628 | 092847 |
| 33450 | 36395315 | 36397764 | 271914, 126070 |
| 33451 | 36499574 | 36501014 | 126070 |
| 33452 | 36553669 | 36555959 | 116863, 092850 |
| 33453 | 36614074 | 36617554 | 054116 |
| 33454 | 36620560 | 36623181 | 116871 |
| 33455 | 36668709 | 36670410 | |
| 33456 | 36688845 | 36691175 | 054118 |
| 33457 | 36705558 | 36706294 | 054118 |
| 33458 | 36709378 | 36710511 | 054118 |
| 33459 | 36711037 | 36711813 | 054118 |
| 33460 | 36713183 | 36714819 | 054118 |
| 33461 | 36734644 | 36735864 | 054118 |
| 33462 | 36744678 | 36745420 | 054118 |
| 33463 | 36756353 | 36756861 | 054118 |
| 33464 | 36765450 | 36767378 | 054118 |
| 33465 | 36771885 | 36772907 | 214193 |
| 33466 | 36788458 | 36789795 | 116883, 142694, 214193 |
| 33467 | 36839353 | 36840790 | 196182 |
| 33468 | 36849999 | 36854135 | 196182 |
| 33469 | 36862687 | 36863833 | 181817 |
| 33470 | 36915773 | 36917011 | 116885 |
| 33471 | 36928871 | 36930871 | 116898 |
| 33472 | 36951924 | 36953061 | |
| 33473 | 37936892 | 37937841 | 233621 |
| 33474 | 37939664 | 37946412 | 163874, 233621 |
| 33475 | 37951665 | 37953213 | |
| 33476 | 37964582 | 37965144 | 163875 |
| 33477 | 37979431 | 37981438 | 163875 |
| 33478 | 37998087 | 38003657 | 163877 |
| 33479 | 38019041 | 38020183 | 163877 |
| 33480 | 38060643 | 38061779 | 134697 |
| 33481 | 38155176 | 38156461 | 116922 |
| 33482 | 38157274 | 38158368 | 134690, 116922 |
| 33483 | 38259327 | 38260167 | 185090 |
| 33484 | 38272660 | 38275073 | 196449, 197982 |
| 33485 | 38324254 | 38326435 | 230955, 204084, 188786 |
| 33486 | 38327706 | 38329057 | 204084 |
| 33487 | 38405596 | 38406657 | 204084 |
| 33488 | 38411022 | 38413517 | 204084 |
| 33489 | 38454680 | 38456364 | 183431 |
| 33490 | 38464936 | 38466947 | 183386 |
| 33491 | 38470520 | 38471487 | 183386 |
| 33492 | 38477777 | 38479232 | 183520 |
| 33493 | 38582917 | 38583965 | 224592 |
| 33494 | 38604136 | 38605157 | |
| 33495 | 38712241 | 38713325 | |
| 33496 | 38752613 | 38753731 | |
| 33497 | 39324447 | 39326523 | 228436, 116954 |
| 33498 | 39338356 | 39339597 | 214114, 228436, 131233 |
| 33499 | 39424483 | 39425104 | 235549 |
| 33500 | 39427131 | 39427687 | |
| 33501 | 39451013 | 39452788 | |
| 33502 | 39455911 | 39458574 | 174574 |
| 33503 | 39491269 | 39492837 | 168653 |
| 33504 | 39569935 | 39573137 | 127603 |
| 33505 | 39576111 | 39576671 | 127603 |
| 33506 | 39582428 | 39583430 | 127603 |
| 33507 | 39597754 | 39599504 | 127603 |
| 33508 | 39608381 | 39608774 | 127603 |
| 33509 | 39618630 | 39621563 | 222378, 127603 |
| 33510 | 39622923 | 39626236 | 127603 |
| 33511 | 39633408 | 39634054 | 127603 |
| 33512 | 39642066 | 39646728 | 127603 |
| 33513 | 39648354 | 39653539 | 127603 |
| 33514 | 39656809 | 39659890 | 127603 |
| 33515 | 39686448 | 39687297 | 127603 |
| 33516 | 39836130 | 39837471 | 127603 |
| 33517 | 39853123 | 39853592 | 127603 |
| 33518 | 39857187 | 39859005 | 127603 |
| 33519 | 39860090 | 39861625 | 127603 |
| 33520 | 39872777 | 39875372 | 127603 |
| 33521 | 39916365 | 39917996 | 127603 |
| 33522 | 40032139 | 40033258 | 201457, 090621, 228060 |
| 33523 | 40040976 | 40042851 | 090621 |
| 33524 | 40204122 | 40205446 | 084072 |
| 33525 | 40264635 | 40266752 | |
| 33526 | 40290881 | 40291789 | |
| 33527 | 40317741 | 40318652 | 043514 |
| 33528 | 40348363 | 40349494 | 043514 |
| 33529 | 40364679 | 40365135 | 236546, 116990 |
| 33530 | 40366238 | 40368364 | 116990 |
| 33531 | 40391892 | 40394095 | |
| 33532 | 40394215 | 40395525 | |
| 33533 | 40399187 | 40401067 | |
| 33534 | 40419984 | 40422559 | 168389 |
| 33535 | 40425261 | 40426538 | 168389 |
| 33536 | 40504515 | 40507624 | 131236 |
| 33537 | 40562289 | 40563100 | 131238 |
| 33538 | 40626505 | 40628697 | 117000 |
| 33539 | 40722414 | 40724716 | 084073, 259943 |
| 33540 | 40733384 | 40734158 | 084073 |
| 33541 | 40839180 | 40840637 | 084070 |
| 33542 | 40845442 | 40845995 | 084070 |
| 33543 | 40854866 | 40856409 | 084070 |
| 33544 | 40887223 | 40888016 | 084070 |
| 33545 | 40915342 | 40916407 | 187801 |
| 33546 | 40942344 | 40943671 | 187815 |
| 33547 | 40973931 | 40975114 | 164002, 238287 |
| 33548 | 40996466 | 40997952 | 117010 |
| 33549 | 41004993 | 41005685 | 117010 |
| 33550 | 41112019 | 41112506 | 117016 |
| 33551 | 41156961 | 41158962 | 066136, 272145 |
| 33552 | 41174669 | 41178574 | 066136 |
| 33553 | 41181212 | 41181747 | 066136 |
| 33554 | 41201417 | 41202358 | 066136 |
| 33555 | 41204809 | 41206650 | 066136 |
| 33556 | 41214452 | 41215920 | 066136 |
| 33557 | 41220473 | 41221244 | 066136 |
| 33558 | 41224142 | 41224813 | 066136 |
| 33559 | 41249140 | 41249717 | 117013 |
| 33560 | 41324705 | 41328820 | 179862 |
| 33561 | 41333923 | 41334564 | 229528 |
| 33562 | 41340721 | 41342137 | 229528 |
| 33563 | 41342509 | 41344366 | |
| 33564 | 41361975 | 41362824 | |
| 33565 | 41384987 | 41385667 | |
| 33566 | 41395740 | 41396276 | |
| 33567 | 41405109 | 41406090 | 228940 |
| 33568 | 41409500 | 41411926 | |
| 33569 | 41413982 | 41415172 | |
| 33570 | 41432876 | 41434177 | |
| 33571 | 41444427 | 41447283 | 171793 |
| 33572 | 41450931 | 41455223 | 171793 |
| 33573 | 41509761 | 41510764 | 010803 |
| 33574 | 41573808 | 41576838 | 010803 |
| 33575 | 41588774 | 41589212 | 010803 |
| 33576 | 41614086 | 41624778 | 010803 |
| 33577 | 41703111 | 41708677 | 235358, 010803 |
| 33578 | 41826676 | 41827079 | |
| 33579 | 41897909 | 41899030 | |
| 33580 | 41933744 | 41934288 | |
| 33581 | 41941036 | 41943251 | |
| 33582 | 41947428 | 41948927 | 127129 |
| 33583 | 41960985 | 41963166 | |
| 33584 | 41964332 | 41965250 | |
| 33585 | 41979941 | 41980428 | 127124 |
| 33586 | 41981679 | 41982424 | 127124 |
| 33587 | 42083690 | 42084128 | 127124 |
| 33588 | 42132868 | 42133357 | 127124 |
| 33589 | 42204601 | 42205244 | 127124 |
| 33590 | 42238494 | 42239413 | 127124 |
| 33591 | 42356348 | 42357669 | 127124 |
| 33592 | 42383609 | 42384614 | 127124 |
| 33593 | 42392658 | 42393346 | 127124 |
| 33594 | 42401633 | 42402393 | 127124 |
| 33595 | 42470362 | 42470984 | 127124 |
| 33596 | 42500754 | 42502463 | 127124 |
| 33597 | 42512255 | 42512845 | |
| 33598 | 42546925 | 42547807 | |
| 33599 | 42612326 | 42613147 | |
| 33600 | 42617400 | 42618935 | |
| 33601 | 42678993 | 42680829 | 198815 |
| 33602 | 42790556 | 42791302 | 198815 |
| 33603 | 42799562 | 42802389 | 227527, 198815 |
| 33604 | 42805610 | 42806277 | |
| 33605 | 42833782 | 42834620 | |
| 33606 | 42897040 | 42897613 | 066185 |
| 33607 | 42919328 | 42921036 | 066185 |
| 33608 | 42921059 | 42923190 | 127125, 066185 |
| 33609 | 42994933 | 42996024 | 186409 |
| 33610 | 43123147 | 43124691 | 171960 |
| 33611 | 43146557 | 43149480 | 065978, 234917 |
| 33612 | 43205797 | 43206635 | 164007 |
| 33613 | 43231368 | 43233705 | 164008, 117385 |
| 33614 | 43282093 | 43283374 | 164010, 177868 |
| 33615 | 43311369 | 43313299 | 164011, 228192 |
| 33616 | 43401423 | 43405988 | 117394 |
| 33617 | 43406150 | 43408716 | 117394 |
| 33618 | 43423364 | 43425418 | 227533, 117394 |
| 33619 | 43700444 | 43702310 | 117395, 243710 |
| 33620 | 43707043 | 43708178 | 117395, 243710 |
| 33621 | 43712291 | 43714331 | 117395, 243710 |
| 33622 | 43715653 | 43716585 | 117395, 243710 |
| 33623 | 43722092 | 43723822 | 117395 |
| 33624 | 43732884 | 43733626 | 117395 |
| 33625 | 43742052 | 43743756 | |
| 33626 | 43778036 | 43778793 | 066056 |
| 33627 | 43810826 | 43811596 | 117400 |
| 33628 | 43813726 | 43815452 | 117400 |
| 33629 | 43823639 | 43824775 | 117399, 234694 |
| 33630 | 43832047 | 43835046 | 066322 |
| 33631 | 43837793 | 43840170 | |
| 33632 | 43854516 | 43856045 | 198198, 229431, 159479 |
| 33633 | 43913529 | 43915135 | 229372, 198198 |
| 33634 | 43917939 | 43920777 | 229348, 178922, 198198 |
| 33635 | 43937328 | 43940373 | |
| 33636 | 43940996 | 43943572 | |
| 33637 | 43959345 | 43960153 | |
| 33638 | 43987924 | 43989842 | |
| 33639 | 43995673 | 43998399 | 142949 |
| 33640 | 44002892 | 44003788 | 142949 |
| 33641 | 44010894 | 44012451 | 142949 |
| 33642 | 44013125 | 44015368 | 142949 |
| 33643 | 44015412 | 44016561 | 142949 |
| 33644 | 44018131 | 44018997 | 142949 |
| 33645 | 44113719 | 44116824 | 066135 |
| 33646 | 44172104 | 44174346 | 236200, 126091 |
| 33647 | 44248578 | 44250300 | 126091 |
| 33648 | 44395259 | 44396344 | 126091 |
| 33649 | 44411337 | 44413648 | 117408, 237950 |
| 33650 | 44435479 | 44436591 | 132768 |
| 33651 | 44439727 | 44441906 | 117410 |
| 33652 | 44443789 | 44446566 | 117411, 117410 |
| 33653 | 44456421 | 44457992 | 159214, 196517, 117411 |
| 33654 | 44495030 | 44497466 | 196517 |
| 33655 | 44508983 | 44510791 | 230615 |
| 33656 | 44532628 | 44533995 | 230615 |
| 33657 | 44678372 | 44679890 | 178028 |
| 33658 | 44683140 | 44684187 | 178028 |
| 33659 | 44748943 | 44750072 | 117419 |
| 33660 | 44759343 | 44760006 | 117419 |
| 33661 | 44762857 | 44764185 | 117419 |
| 33662 | 44792490 | 44794058 | 117419 |
| 33663 | 44820156 | 44821455 | 117419 |
| 33664 | 45096758 | 45098931 | 187147 |
| 33665 | 45111067 | 45111946 | 187147, 126106 |
| 33666 | 45125805 | 45126366 | 126106 |
| 33667 | 45138972 | 45140794 | 198520, 126106 |
| 33668 | 45142164 | 45143296 | 198520 |
| 33669 | 45143990 | 45144816 | 198520 |
| 33670 | 45147029 | 45148588 | 198520 |
| 33671 | 45195784 | 45197304 | 200169 |
| 33672 | 45204842 | 45205586 | 142945 |
| 33673 | 45240262 | 45242517 | 264294, 200913, 142937, |
| | | | 225721 |
| 33674 | 45264961 | 45266916 | 173846 |
| 33675 | 45271830 | 45275601 | 222009, 188396 |
| 33676 | 45278310 | 45280010 | 222009 |
| 33677 | 45284734 | 45286458 | 117425, 202444 |
| 33678 | 45412333 | 45413224 | 070785 |
| 33679 | 45450885 | 45452682 | 070785 |
| 33680 | 45476193 | 45479281 | 126088, 126107 |
| 33681 | 45600109 | 45600437 | 162415 |
| 33682 | 45637596 | 45638304 | 162415 |
| 33683 | 45768578 | 45770689 | 162415 |
| 33684 | 45801380 | 45802093 | 132781 |
| 33685 | 45804775 | 45807020 | 132773, 132781 |
| 33686 | 45813536 | 45815141 | 070759 |
| 33687 | 45868893 | 45869488 | 070759 |
| 33688 | 45955550 | 45957402 | 070759 |
| 33689 | 45965301 | 45966980 | 132763, 236624 |
| 33690 | 45967458 | 45968301 | 132763 |
| 33691 | 45986358 | 45988902 | 117450 |
| 33692 | 46007954 | 46009030 | |
| 33693 | 46009252 | 46009987 | |
| 33694 | 46010589 | 46011775 | |
| 33695 | 46015608 | 46017572 | 117448 |
| 33696 | 46022559 | 46023781 | 117448 |
| 33697 | 46049114 | 46051188 | 132780 |
| 33698 | 46123657 | 46124255 | 159592 |
| 33699 | 46139453 | 46140583 | 159592 |
| 33700 | 46150081 | 46155060 | 159596, 159592 |
| 33701 | 46215311 | 46217138 | 197429 |
| 33702 | 46267758 | 46270816 | 086015 |
| 33703 | 46336348 | 46338497 | 086015 |
| 33704 | 46357125 | 46359109 | 086015 |
| 33705 | 46419864 | 46420703 | 086015 |
| 33706 | 46440394 | 46441139 | 086015 |
| 33707 | 46444459 | 46445427 | 086015 |
| 33708 | 46527620 | 46529124 | 117461 |
| 33709 | 46549748 | 46551288 | 117461 |
| 33710 | 46597389 | 46599487 | 117461 |
| 33711 | 46648201 | 46650208 | 117472 |
| 33712 | 46662970 | 46664991 | 085998 |
| 33713 | 46712707 | 46713142 | |
| 33714 | 46766528 | 46770163 | 173660, 132128 |
| 33715 | 46805432 | 46807579 | 117481 |
| 33716 | 46859465 | 46860872 | 117480 |
| 33717 | 46867073 | 46868175 | 117480 |
| 33718 | 46907969 | 46908957 | 232022 |
| 33719 | 46992323 | 46994236 | |
| 33720 | 47069051 | 47070320 | 079277 |
| 33721 | 47081584 | 47082850 | 142961, 079277 |
| 33722 | 47133038 | 47134751 | 186118, 123472, 159658 |
| 33723 | 47183842 | 47185286 | 159658 |
| 33724 | 47201370 | 47201879 | |
| 33725 | 47407742 | 47408188 | |
| 33726 | 47413712 | 47415303 | |
| 33727 | 47478393 | 47478919 | 186377 |
| 33728 | 47593942 | 47595097 | 225506 |
| 33729 | 47644427 | 47645483 | 224805, 225506 |
| 33730 | 47655051 | 47659097 | 162366 |
| 33731 | 47779474 | 47780811 | 123473 |
| 33732 | 47798417 | 47801202 | 162368 |
| 33733 | 47902069 | 47904174 | 186564 |
| 33734 | 47942693 | 47944710 | |
| 33735 | 47973426 | 47974943 | |
| 33736 | 47988433 | 47989128 | |
| 33737 | 48024146 | 48025387 | |
| 33738 | 48025689 | 48026705 | |
| 33739 | 48026785 | 48028036 | |
| 33740 | 48035298 | 48035930 | |
| 33741 | 48162543 | 48163235 | 227947 |
| 33742 | 48174674 | 48176828 | |
| 33743 | 48194037 | 48194813 | |
| 33744 | 48197941 | 48199070 | |
| 33745 | 48341493 | 48342438 | 269113 |
| 33746 | 48359683 | 48361599 | 269113 |
| 33747 | 48378743 | 48379788 | 269113 |
| 33748 | 48406863 | 48409106 | 269113 |
| 33749 | 48428422 | 48430859 | 269113 |
| 33750 | 48434716 | 48436154 | 269113 |
| 33751 | 48462222 | 48463743 | 269113 |
| 33752 | 48472669 | 48473290 | |
| 33753 | 48688060 | 48688883 | 117834 |
| 33754 | 49137234 | 49137779 | 186094 |
| 33755 | 49383294 | 49384416 | 186094 |
| 33756 | 50646621 | 50647359 | 162374 |
| 33757 | 50680215 | 50681260 | 233407 |
| 33758 | 50940497 | 50941037 | 185104 |
| 33759 | 50992946 | 50993604 | 185104 |
| 33760 | 51294958 | 51295615 | 185104 |
| 33761 | 51424790 | 51426875 | 123080, 185104 |
| 33762 | 51433627 | 51436665 | 123080 |
| 33763 | 51442114 | 51446332 | |
| 33764 | 51681211 | 51683159 | 203356 |
| 33765 | 51700906 | 51704450 | 123091 |
| 33766 | 51749616 | 51750455 | |
| 33767 | 51779295 | 51781277 | 085831 |
| 33768 | 51795781 | 51797596 | 085831, 261664 |
| 33769 | 51983426 | 51985406 | 227070, 085832 |
| 33770 | 52026167 | 52027068 | 227070 |
| 33771 | 52029414 | 52030465 | 227742 |
| 33772 | 52034586 | 52036028 | 227742 |
| 33773 | 52082435 | 52083771 | 117859 |
| 33774 | 52085915 | 52089714 | 117859 |
| 33775 | 52101963 | 52104011 | 117859 |
| 33776 | 52154983 | 52156831 | 117859 |
| 33777 | 52158216 | 52160166 | 117859 |
| 33778 | 52192161 | 52192917 | 117859 |
| 33779 | 52194478 | 52196273 | 117859 |
| 33780 | 52343448 | 52345683 | 078618 |
| 33781 | 52433476 | 52436584 | 169213 |
| 33782 | 52442563 | 52443375 | 169213 |
| 33783 | 52498332 | 52499882 | 223390, 198841, 117862 |
| 33784 | 52520076 | 52522887 | 134717, 117862 |
| 33785 | 52606567 | 52608801 | 157077 |
| 33786 | 52810820 | 52811465 | 154222, 157077 |
| 33787 | 52829952 | 52832090 | 154222 |
| 33788 | 52869387 | 52870997 | 134748, 085840 |
| 33789 | 53017658 | 53019878 | 134744 |
| 33790 | 53067630 | 53068922 | 116157 |
| 33791 | 53117083 | 53118533 | 182183 |
| 33792 | 53124357 | 53124846 | 182183 |
| 33793 | 53130874 | 53131826 | 182183 |
| 33794 | 53163176 | 53164857 | 162377 |
| 33795 | 53166225 | 53167048 | |
| 33796 | 53168042 | 53169759 | |
| 33797 | 53172713 | 53174910 | 233664 |
| 33798 | 53191948 | 53193450 | 162378 |
| 33799 | 53280091 | 53280571 | 162378 |
| 33800 | 53308176 | 53309398 | 203995 |
| 33801 | 53371475 | 53373747 | 121310 |
| 33802 | 53378417 | 53379612 | 121310 |
| 33803 | 53381620 | 53382838 | 121310 |
| 33804 | 53384416 | 53387852 | 230953, 121310 |
| 33805 | 53391017 | 53391919 | 231866, 121310 |
| 33806 | 53392256 | 53394138 | 116171, 121310 |
| 33807 | 53490577 | 53491693 | 116171 |
| 33808 | 53554784 | 53555946 | 162383 |
| 33809 | 53577820 | 53578539 | 162383 |
| 33810 | 53611381 | 53612579 | |
| 33811 | 53631030 | 53631584 | |
| 33812 | 53642188 | 53642932 | |
| 33813 | 53647625 | 53649573 | |
| 33814 | 53655889 | 53656559 | |
| 33815 | 53661004 | 53664523 | 157184 |
| 33816 | 53684950 | 53687204 | 259818, 162384 |
| 33817 | 53703372 | 53704849 | 226754, 162385 |
| 33818 | 54302828 | 54304763 | 058804 |
| 33819 | 54348571 | 54349943 | 058799 |
| 33820 | 54354746 | 54356629 | 058799 |
| 33821 | 54359375 | 54360252 | 211452 |
| 33822 | 54360363 | 54360729 | 211452 |
| 33823 | 54362390 | 54363302 | 211452 |
| 33824 | 54364301 | 54366024 | 211452 |
| 33825 | 54372950 | 54373408 | 211452 |
| 33826 | 54375346 | 54376137 | 211452 |
| 33827 | 54410557 | 54412809 | 116212, 081870 |
| 33828 | 54422263 | 54423581 | 116212 |
| 33829 | 54432950 | 54434212 | 116212 |
| 33830 | 54446795 | 54447395 | |
| 33831 | 54447629 | 54449012 | |
| 33832 | 54481799 | 54482681 | 203985 |
| 33833 | 54517617 | 54519990 | 264208, 116205, 116209 |
| 33834 | 54546094 | 54546920 | 116205 |
| 33835 | 54547141 | 54547749 | 116205 |
| 33836 | 54586791 | 54587678 | |
| 33837 | 54589938 | 54590788 | |
| 33838 | 54665044 | 54667021 | 215883, 256407, 116221 |
| 33839 | 54737807 | 54741898 | 157216 |
| 33840 | 54742588 | 54745037 | 157216 |
| 33841 | 54813305 | 54814345 | 157216 |
| 33842 | 54830675 | 54831110 | 157216 |
| 33843 | 54842389 | 54843315 | 157216 |
| 33844 | 54849053 | 54849400 | 157216 |
| 33845 | 54870868 | 54871793 | 157216 |
| 33846 | 54871907 | 54874671 | 157216 |
| 33847 | 54890606 | 54891223 | |
| 33848 | 54949481 | 54950020 | |
| 33849 | 54953072 | 54955501 | |
| 33850 | 55046857 | 55047773 | 162390 |
| 33851 | 55088999 | 55089683 | 162391, 162390, 230728 |
| 33852 | 55117175 | 55117680 | 271723, 184313 |
| 33853 | 55180824 | 55183133 | 243725, 271723 |
| 33854 | 55259198 | 55261863 | 237453, 006555 |
| 33855 | 55263736 | 55265715 | 006555 |
| 33856 | 55265788 | 55267532 | 006555 |
| 33857 | 55269133 | 55271782 | 162398 |
| 33858 | 55321173 | 55324551 | 116133 |
| 33859 | 55330116 | 55331855 | 116133 |
| 33860 | 55332033 | 55333845 | 116133 |
| 33861 | 55334508 | 55335557 | 116133 |
| 33862 | 55339836 | 55342233 | 116133 |
| 33863 | 55347871 | 55354650 | 233203, 242396, 116133 |
| 33864 | 55384950 | 55386398 | |
| 33865 | 55388405 | 55389172 | |
| 33866 | 55400221 | 55401390 | |
| 33867 | 55415788 | 55416932 | |
| 33868 | 55501256 | 55502060 | |
| 33869 | 55504441 | 55507101 | 169174 |
| 33870 | 55518288 | 55519788 | 169174 |
| 33871 | 55530277 | 55531106 | 169174 |
| 33872 | 55593664 | 55595282 | 162402 |
| 33873 | 55670529 | 55671153 | 162402 |
| 33874 | 55679483 | 55682070 | 162402 |
| 33875 | 55698793 | 55699922 | 231090 |
| 33876 | 55893681 | 55896270 | |
| 33877 | 55908573 | 55912104 | |
| 33878 | 56149551 | 56150384 | 234810 |
| 33879 | 56183273 | 56185919 | 234810 |
| 33880 | 56279206 | 56279864 | |
| 33881 | 56750413 | 56751394 | |
| 33882 | 56794769 | 56799124 | |
| 33883 | 56840098 | 56841348 | |
| 33884 | 56874106 | 56878551 | |
| 33885 | 56878801 | 56882730 | 223956 |
| 33886 | 56897743 | 56898803 | |
| 33887 | 56910496 | 56911493 | |
| 33888 | 56911594 | 56916400 | |
| 33889 | 56916790 | 56919711 | |
| 33890 | 56921966 | 56922941 | |
| 33891 | 56926839 | 56927594 | |
| 33892 | 56935628 | 56936569 | |
| 33893 | 56938410 | 56940408 | |
| 33894 | 56941201 | 56945713 | |
| 33895 | 56955887 | 56957436 | |
| 33896 | 56972444 | 56975271 | 162407 |
| 33897 | 57009317 | 57010194 | 162407 |
| 33898 | 57011033 | 57012057 | 162407 |
| 33899 | 57026961 | 57027763 | 162407 |
| 33900 | 57029024 | 57029718 | 162407 |
| 33901 | 57031437 | 57032574 | 162407 |
| 33902 | 57035122 | 57035848 | 162407 |
| 33903 | 57042521 | 57045828 | 162407 |
| 33904 | 57056262 | 57056586 | 162407 |
| 33905 | 57153769 | 57155235 | 162409 |
| 33906 | 57221339 | 57222070 | 187889 |
| 33907 | 57238032 | 57241015 | 187889 |
| 33908 | 57242203 | 57242554 | 187889 |
| 33909 | 57245886 | 57246470 | 187889 |
| 33910 | 57253790 | 57254719 | 187889 |
| 33911 | 57262886 | 57264684 | 187889 |
| 33912 | 57276586 | 57277510 | 187889 |
| 33913 | 57282199 | 57285698 | 187889 |
| 33914 | 57297444 | 57299550 | |
| 33915 | 57319981 | 57323141 | 157131 |
| 33916 | 57362554 | 57364095 | 157131 |
| 33917 | 57367302 | 57368055 | 157131 |
| 33918 | 57372997 | 57375118 | 157131 |
| 33919 | 57375550 | 57378433 | 157131 |
| 33920 | 57384894 | 57386705 | |
| 33921 | 57387223 | 57388137 | |
| 33922 | 57390331 | 57391821 | |
| 33923 | 57423741 | 57425197 | 021852 |
| 33924 | 57428588 | 57435763 | 236341, 221591, 021852 |
| 33925 | 57701067 | 57702033 | 173406 |
| 33926 | 57719511 | 57720265 | 173406 |
| 33927 | 57744098 | 57744897 | 173406 |
| 33928 | 57744975 | 57749121 | 173406 |
| 33929 | 58860866 | 58861775 | 173406 |
| 33930 | 58898888 | 58899516 | 162600, 173406 |
| 33931 | 58911844 | 58913549 | 162600, 173406 |
| 33932 | 58922139 | 58923363 | 162600, 173406 |
| 33933 | 59011053 | 59012874 | 162600, 173406 |
| 33934 | 59039165 | 59040015 | |
| 33935 | 59049552 | 59051546 | |
| 33936 | 59057719 | 59059192 | |
| 33937 | 59125757 | 59126261 | 162601 |
| 33938 | 59164461 | 59166023 | 162601 |
| 33939 | 59180737 | 59181088 | |
| 33940 | 59218108 | 59222482 | |
| 33941 | 59227489 | 59231044 | 264081 |
| 33942 | 59244624 | 59252001 | 177606, 234807 |
| 33943 | 59276833 | 59277308 | 234807 |
| 33944 | 59280080 | 59282883 | 234807 |
| 33945 | 59346371 | 59350070 | 232453, 234807 |
| 33946 | 59350790 | 59351542 | 232453, 234807 |
| 33947 | 59364736 | 59365320 | 234807 |
| 33948 | 59368675 | 59370440 | |
| 33949 | 59430628 | 59431345 | |
| 33950 | 59433905 | 59434874 | |
| 33951 | 59436586 | 59437975 | |
| 33952 | 59448075 | 59448812 | |
| 33953 | 59480924 | 59482021 | |
| 33954 | 59485237 | 59486783 | 230812 |
| 33955 | 59509334 | 59509940 | 230812 |
| 33956 | 59517734 | 59518315 | |
| 33957 | 59602148 | 59603262 | 224609 |
| 33958 | 59608009 | 59609544 | 224609 |
| 33959 | 59611897 | 59613192 | 224609 |
| 33960 | 59616238 | 59617800 | 224609 |
| 33961 | 59669542 | 59670314 | |
| 33962 | 59680288 | 59681167 | |
| 33963 | 59684167 | 59684719 | |
| 33964 | 59758138 | 59759298 | |
| 33965 | 59761199 | 59765232 | 172456 |
| 33966 | 59781406 | 59783818 | 172456 |
| 33967 | 59858412 | 59860020 | 172456 |
| 33968 | 59888462 | 59889711 | 172456 |
| 33969 | 59891563 | 59894201 | 172456 |
| 33970 | 59896199 | 59900273 | 172456 |
| 33971 | 59945563 | 59946623 | 172456 |
| 33972 | 59964221 | 59967052 | 172456 |
| 33973 | 59967873 | 59968523 | 172456 |
| 33974 | 59972355 | 59973232 | 172456 |
| 33975 | 59982417 | 59982903 | 172456 |
| 33976 | 60008379 | 60009869 | 172456 |
| 33977 | 60015322 | 60016581 | 172456 |
| 33978 | 60137560 | 60140982 | 172456 |
| 33979 | 60162840 | 60163370 | 172456 |
| 33980 | 60195320 | 60197978 | 172456 |
| 33981 | 60279856 | 60281409 | 134709 |
| 33982 | 60322126 | 60323037 | 134709 |
| 33983 | 60374104 | 60374937 | 134716 |
| 33984 | 60391768 | 60393317 | 134716 |
| 33985 | 60401107 | 60403353 | |
| 33986 | 60730964 | 60732469 | |
| 33987 | 60739937 | 60741133 | |
| 33988 | 60746986 | 60747720 | |
| 33989 | 61038114 | 61040502 | 226476 |
| 33990 | 61273762 | 61274972 | 231252 |
| 33991 | 61294484 | 61295104 | |
| 33992 | 61332260 | 61333121 | 162599 |
| 33993 | 61508361 | 61509655 | 162599 |
| 33994 | 61515384 | 61517364 | 162599 |
| 33995 | 61519239 | 61520207 | 162599 |
| 33996 | 61522428 | 61523889 | 162599 |
| 33997 | 61524156 | 61524566 | 162599 |
| 33998 | 61542152 | 61544107 | 162599 |
| 33999 | 61545248 | 61545979 | 162599 |
| 34000 | 61547029 | 61550704 | 263380, 162599 |
| 34001 | 61580053 | 61580634 | 162599 |
| 34002 | 61584153 | 61585493 | 162599 |
| 34003 | 61585699 | 61587802 | 162599 |
| 34004 | 61587856 | 61588664 | 162599 |
| 34005 | 61604856 | 61605427 | 162599 |
| 34006 | 61606075 | 61609223 | 162599 |
| 34007 | 61617528 | 61619042 | 162599 |
| 34008 | 61648984 | 61651067 | 162599 |
| 34009 | 61668344 | 61669624 | 162599 |
| 34010 | 61678270 | 61682626 | 162599 |
| 34011 | 61718452 | 61719594 | 237853, 162599 |
| 34012 | 61766490 | 61768414 | 162599 |
| 34013 | 61802814 | 61805100 | 162599 |
| 34014 | 61918268 | 61919546 | 162599 |
| 34015 | 62190005 | 62191979 | 162604 |
| 34016 | 62207308 | 62209364 | 271200, 132849 |
| 34017 | 62302672 | 62307084 | 132849 |
| 34018 | 62438732 | 62440752 | 132849 |
| 34019 | 62558730 | 62559418 | 132849 |
| 34020 | 62664141 | 62664875 | 240563 |
| 34021 | 62707080 | 62707718 | 132854 |
| 34022 | 62783774 | 62785301 | 132854 |
| 34023 | 62900959 | 62903750 | 162607 |
| 34024 | 63051117 | 63051565 | 116641 |
| 34025 | 63059416 | 63060144 | 116641 |
| 34026 | 63062187 | 63065725 | 132855, 116641 |
| 34027 | 63087412 | 63088244 | 116641 |
| 34028 | 63153011 | 63154962 | 235545, 116641 |
| 34029 | 63155046 | 63157338 | 235545 |
| 34030 | 63199204 | 63202035 | |
| 34031 | 63249159 | 63250045 | 125703 |
| 34032 | 63782525 | 63783287 | 224209 |
| 34033 | 63832705 | 63834009 | 088035 |
| 34034 | 63988522 | 63989446 | 203965, 142856 |
| 34035 | 64057979 | 64060536 | 079739, 142856 |
| 34036 | 64065658 | 64066334 | 079739 |
| 34037 | 64076405 | 64077341 | 079739 |
| 34038 | 64078273 | 64078766 | 079739 |
| 34039 | 64106393 | 64107397 | 079739 |
| 34040 | 64127812 | 64129789 | |
| 34041 | 64139800 | 64141070 | |
| 34042 | 64239352 | 64240394 | 185483 |
| 34043 | 64636930 | 64638846 | 223949, 185483 |
| 34044 | 64886651 | 64887885 | |
| 34045 | 64935309 | 64936632 | 158966 |
| 34046 | 64944498 | 64944991 | 158966 |
| 34047 | 65001727 | 65003125 | 158966 |
| 34048 | 65362144 | 65365911 | 162434 |
| 34049 | 65375376 | 65378906 | 162434 |
| 34050 | 65380450 | 65384557 | 234784, 162434 |
| 34051 | 65398164 | 65401178 | 162434 |
| 34052 | 65401398 | 65402381 | 162434 |
| 34053 | 65402579 | 65403148 | 162434 |
| 34054 | 65430541 | 65432972 | 162434 |
| 34055 | 65452808 | 65453946 | 226891 |
| 34056 | 65455136 | 65455580 | 226891 |
| 34057 | 65467421 | 65469711 | 272506, 226891 |
| 34058 | 65505107 | 65505720 | |
| 34059 | 65531678 | 65535015 | 231485 |
| 34060 | 65555849 | 65556476 | |
| 34061 | 65612726 | 65615445 | 162433 |
| 34062 | 65624014 | 65626309 | 162433 |
| 34063 | 65634552 | 65635321 | 162433 |
| 34064 | 65635340 | 65636783 | 162433 |
| 34065 | 65665969 | 65666725 | 162433 |
| 34066 | 65719439 | 65720977 | 116675 |
| 34067 | 65861542 | 65862463 | 116675 |
| 34068 | 65873479 | 65874058 | 116675 |
| 34069 | 65883295 | 65884059 | |
| 34070 | 65886083 | 65892396 | 213625, 116678 |
| 34071 | 65936350 | 65938096 | 116678 |
| 34072 | 65941847 | 65942552 | 116678 |
| 34073 | 65978004 | 65980428 | 116678 |
| 34074 | 65990724 | 65992308 | 116678 |
| 34075 | 66062473 | 66062871 | 116678 |
| 34076 | 66153021 | 66153944 | |
| 34077 | 66612648 | 66613380 | 184588 |
| 34078 | 66635143 | 66635516 | 184588 |
| 34079 | 66736205 | 66738496 | 184588 |
| 34080 | 66758555 | 66761347 | 184588 |
| 34081 | 66765692 | 66767681 | 184588 |
| 34082 | 66814706 | 66815977 | 184588 |
| 34083 | 66878171 | 66878782 | |
| 34084 | 66985698 | 66986121 | |
| 34085 | 67008027 | 67009763 | 118473 |
| 34086 | 67390087 | 67391807 | 198160, 152763 |
| 34087 | 67395332 | 67397556 | 198160 |
| 34088 | 67409160 | 67411994 | 198160 |
| 34089 | 67518355 | 67520557 | 116704 |
| 34090 | 67635519 | 67635996 | 162594, 203963 |
| 34091 | 67833082 | 67833991 | 081985 |
| 34092 | 67894443 | 67897738 | 142864 |
| 34093 | 67962004 | 67962368 | |
| 34094 | 67987493 | 67988886 | |
| 34095 | 67997846 | 67999704 | 235200 |
| 34096 | 68000055 | 68004001 | |
| 34097 | 68005257 | 68006822 | 207504 |
| 34098 | 68017744 | 68018925 | |
| 34099 | 68026354 | 68028657 | 238139 |
| 34100 | 68035073 | 68039934 | |
| 34101 | 68040411 | 68042609 | |
| 34102 | 68043931 | 68044959 | |
| 34103 | 68079965 | 68080817 | |
| 34104 | 68083996 | 68085366 | |
| 34105 | 68095076 | 68095884 | |
| 34106 | 68096114 | 68096860 | |
| 34107 | 68097204 | 68099413 | |
| 34108 | 68110946 | 68113554 | |
| 34109 | 68147455 | 68158355 | 116717 |
| 34110 | 68165845 | 68167026 | |
| 34111 | 68183069 | 68184128 | 172380 |
| 34112 | 68189405 | 68192279 | 172380 |
| 34113 | 68213354 | 68216948 | 172380 |
| 34114 | 68224782 | 68227054 | 172380 |
| 34115 | 68279426 | 68280908 | 172380 |
| 34116 | 68284399 | 68285244 | 172380 |
| 34117 | 68295130 | 68299766 | 232284, 172380 |
| 34118 | 68304116 | 68307343 | 232284 |
| 34119 | 68315645 | 68317102 | 232284 |
| 34120 | 68355420 | 68356480 | 232284 |
| 34121 | 68611162 | 68612159 | 116729, 232284 |
| 34122 | 68639090 | 68639574 | 116729, 232284 |
| 34123 | 68639754 | 68640117 | 116729, 232284 |
| 34124 | 68662498 | 68665497 | 116729, 232284 |
| 34125 | 68670290 | 68672270 | 116729 |
| 34126 | 68696686 | 68698673 | 116729 |
| 34127 | 68735325 | 68735953 | |
| 34128 | 68808235 | 68808847 | |
| 34129 | 68835915 | 68836731 | |
| 34130 | 68849189 | 68851571 | |
| 34131 | 69637732 | 69638425 | 223883 |
| 34132 | 69734658 | 69739256 | |
| 34133 | 70621600 | 70622649 | 066557 |
| 34134 | 70670392 | 70672614 | 116754, 066557 |
| 34135 | 70686641 | 70688244 | 228988, 116754 |
| 34136 | 70785323 | 70785867 | 118454 |
| 34137 | 70804560 | 70805024 | 118454 |
| 34138 | 70818887 | 70821133 | 197568, 118454 |
| 34139 | 70875653 | 70879511 | 116761 |
| 34140 | 70969810 | 70970775 | |
| 34141 | 71545683 | 71547739 | 229956, 132485 |
| 34142 | 72383953 | 72384930 | 172260 |
| 34143 | 72748579 | 72751282 | |
| 34144 | 72832033 | 72832553 | |
| 34145 | 72875704 | 72876509 | |
| 34146 | 74663373 | 74664390 | 259030, 254685, 116783, |
| | | | 162620 |
| 34147 | 75194747 | 75199828 | 162623, 116791 |
| 34148 | 75214249 | 75215315 | 162623 |
| 34149 | 76189399 | 76192238 | 117054, 178193 |
| 34150 | 76251032 | 76253074 | 206620, 137955, 117054 |
| 34151 | 76256723 | 76257126 | 137955 |
| 34152 | 76320562 | 76321052 | 057468 |
| 34153 | 77139413 | 77140718 | |
| 34154 | 77496623 | 77497617 | 117069 |
| 34155 | 77586617 | 77587672 | 142892 |
| 34156 | 77599692 | 77600487 | 142892 |
| 34157 | 77684407 | 77685538 | 142892 |
| 34158 | 78001898 | 78003511 | 154027 |
| 34159 | 78004340 | 78006095 | 154027 |
| 34160 | 78147239 | 78150197 | 036549 |
| 34161 | 78222956 | 78223849 | 077254 |
| 34162 | 78224183 | 78225709 | 077254 |
| 34163 | 78227588 | 78228109 | |
| 34164 | 78241924 | 78242309 | |
| 34165 | 78244711 | 78246121 | 180488, 222849 |
| 34166 | 78289451 | 78290304 | 180488 |
| 34167 | 78353743 | 78355095 | 162614, 235927 |
| 34168 | 78365254 | 78365792 | 162614 |
| 34169 | 78443678 | 78446017 | 162616, 137960, 162613 |
| 34170 | 78469502 | 78472228 | 273338, 162616, 137960 |
| 34171 | 78510499 | 78513695 | 137960 |
| 34172 | 78534359 | 78535246 | 137960 |
| 34173 | 78535469 | 78537548 | 137960 |
| 34174 | 78544604 | 78545740 | 137960 |
| 34175 | 78554624 | 78555283 | 251958, 137960 |
| 34176 | 78566539 | 78568125 | 137960 |
| 34177 | 78607708 | 78611384 | |
| 34178 | 78687517 | 78690060 | |
| 34179 | 78693128 | 78693899 | |
| 34180 | 78750001 | 78750783 | 237413 |
| 34181 | 78769252 | 78769832 | 122420, 237413 |
| 34182 | 78804664 | 78807491 | 122420, 237413 |
| 34183 | 78937168 | 78940781 | 122420 |
| 34184 | 78956441 | 78957759 | 122420 |
| 34185 | 79115272 | 79116202 | 137965 |
| 34186 | 80578961 | 80579590 | |
| 34187 | 81121602 | 81123851 | |
| 34188 | 81348029 | 81349092 | |
| 34189 | 81607778 | 81608774 | |
| 34190 | 81639617 | 81641815 | |
| 34191 | 81644833 | 81645555 | |
| 34192 | 82048081 | 82048666 | 117114 |
| 34193 | 82265581 | 82269907 | 117114 |
| 34194 | 82273820 | 82275119 | 117114 |
| 34195 | 82564674 | 82565496 | |
| 34196 | 82787359 | 82787903 | |
| 34197 | 82929980 | 82931053 | |
| 34198 | 82981262 | 82982183 | |
| 34199 | 83577876 | 83578857 | 230817 |
| 34200 | 83966261 | 83967212 | |
| 34201 | 83974256 | 83975390 | |
| 34202 | 84158008 | 84160351 | 233008 |
| 34203 | 84284065 | 84285164 | 233008 |
| 34204 | 84325947 | 84327060 | 233008 |
| 34205 | 84366343 | 84368611 | 137941 |
| 34206 | 84450462 | 84451392 | 137941 |
| 34207 | 84464193 | 84465412 | 137941 |
| 34208 | 84542636 | 84544507 | 142875, 271576 |
| 34209 | 84555071 | 84557318 | 142875 |
| 34210 | 84568250 | 84570102 | 142875 |
| 34211 | 84719204 | 84720236 | |
| 34212 | 84726278 | 84727118 | |
| 34213 | 84763900 | 84765056 | 203943 |
| 34214 | 84831109 | 84832364 | 240520, 203943 |
| 34215 | 84944592 | 84946115 | 117133 |
| 34216 | 84958256 | 84958961 | 117133 |
| 34217 | 84970213 | 84973146 | 122432, 174021 |
| 34218 | 85037916 | 85040430 | 117151 |
| 34219 | 85086195 | 85087153 | |
| 34220 | 85155366 | 85156928 | 117155 |
| 34221 | 85163590 | 85164936 | |
| 34222 | 85170511 | 85171688 | |
| 34223 | 85184551 | 85187578 | |
| 34224 | 85189982 | 85190674 | |
| 34225 | 85299710 | 85301310 | 171517 |
| 34226 | 85305133 | 85305836 | 171517 |
| 34227 | 85353066 | 85353654 | 171517 |
| 34228 | 85600818 | 85601210 | |
| 34229 | 85603310 | 85603938 | |
| 34230 | 85665421 | 85668593 | 097096 |
| 34231 | 85724410 | 85725871 | 162642 |
| 34232 | 85740444 | 85742820 | 223653, 142867 |
| 34233 | 85778849 | 85781091 | 223653 |
| 34234 | 85785895 | 85789675 | 153904, 223653 |
| 34235 | 85790191 | 85796691 | 153904, 223653 |
| 34236 | 85804687 | 85805866 | 153904, 223653 |
| 34237 | 85835016 | 85836118 | 153904, 223653 |
| 34238 | 85836513 | 85837323 | 153904, 223653 |
| 34239 | 85838336 | 85838992 | 153904, 223653 |
| 34240 | 85839772 | 85840328 | 153904, 223653 |
| 34241 | 85851337 | 85852411 | 153904, 223653 |
| 34242 | 85868610 | 85869219 | 153904, 223653 |
| 34243 | 85872180 | 85874086 | 153904, 223653 |
| 34244 | 85903813 | 85904622 | 153904, 223653 |
| 34245 | 85925939 | 85926831 | 153904 |
| 34246 | 85928802 | 85931595 | 153904 |
| 34247 | 86010267 | 86011033 | 153904 |
| 34248 | 86041580 | 86044820 | 272691, 153904 |
| 34249 | 86045017 | 86052212 | 142871 |
| 34250 | 86064530 | 86065150 | 230285 |
| 34251 | 86070743 | 86075290 | |
| 34252 | 86076511 | 86079624 | |
| 34253 | 86088614 | 86089192 | |
| 34254 | 86093988 | 86095687 | |
| 34255 | 86173038 | 86174890 | 117174 |
| 34256 | 87169161 | 87171753 | 261737, 097033 |
| 34257 | 87238373 | 87241338 | |
| 34258 | 87253994 | 87255313 | |
| 34259 | 87258357 | 87259375 | |
| 34260 | 87277219 | 87277679 | |
| 34261 | 87289379 | 87289969 | |
| 34262 | 87325590 | 87327688 | |
| 34263 | 87379016 | 87381954 | 153936, 183291 |
| 34264 | 87403849 | 87404685 | 153936 |
| 34265 | 87597371 | 87598228 | 153936, 267561, 267272 |
| 34266 | 87691329 | 87692534 | |
| 34267 | 87793202 | 87795996 | 143013 |
| 34268 | 87796513 | 87798502 | 143013 |
| 34269 | 87800186 | 87801725 | 143013 |
| 34270 | 87828158 | 87829017 | 227290 |
| 34271 | 88149049 | 88150721 | |
| 34272 | 88186266 | 88186778 | |
| 34273 | 88260265 | 88262613 | |
| 34274 | 88394223 | 88394829 | |
| 34275 | 88466563 | 88467707 | |
| 34276 | 88569680 | 88570889 | |
| 34277 | 88578744 | 88579582 | |
| 34278 | 88640536 | 88642503 | |
| 34279 | 88645255 | 88647068 | |
| 34280 | 88877938 | 88878444 | |
| 34281 | 89112775 | 89113884 | 237505 |
| 34282 | 89148807 | 89151290 | 065243, 237505 |
| 34283 | 89356385 | 89358320 | 137947 |
| 34284 | 89412899 | 89413587 | 137944 |
| 34285 | 89457002 | 89459817 | 213516, 137944 |
| 34286 | 89487306 | 89489390 | 117226 |
| 34287 | 89510555 | 89511743 | |
| 34288 | 89529670 | 89531986 | 117228 |
| 34289 | 89535367 | 89536338 | |
| 34290 | 89591059 | 89592739 | 162645 |
| 34291 | 89594332 | 89595608 | 162645 |
| 34292 | 89638129 | 89639310 | 213512 |
| 34293 | 89640787 | 89643031 | 213512 |
| 34294 | 89664049 | 89665072 | 162654 |
| 34295 | 89739888 | 89740953 | |
| 34296 | 89775364 | 89777939 | |
| 34297 | 89989268 | 89991348 | 197147 |
| 34298 | 90018010 | 90019144 | 197147 |
| 34299 | 90286015 | 90289038 | 171492, 272931, 271949 |
| 34300 | 90292120 | 90292629 | 271949, 171492 |
| 34301 | 90301638 | 90303116 | 271949, 171492 |
| 34302 | 90308213 | 90310078 | 271949, 171492 |
| 34303 | 90330772 | 90332795 | 271949, 171492 |
| 34304 | 90336512 | 90338717 | 271949, 171492 |
| 34305 | 90372003 | 90372994 | 271949, 171492 |
| 34306 | 90415486 | 90416385 | |
| 34307 | 90459712 | 90461699 | 162664, 228175 |
| 34308 | 90590619 | 90591277 | |
| 34309 | 91244981 | 91245635 | |
| 34310 | 91315846 | 91317389 | 225446, 233593 |
| 34311 | 91384597 | 91385311 | 122482 |
| 34312 | 91480719 | 91482302 | 122482 |
| 34313 | 91486144 | 91488648 | 122482 |
| 34314 | 91530718 | 91531692 | |
| 34315 | 91586367 | 91587617 | |
| 34316 | 91852644 | 91853268 | 162669 |
| 34317 | 91966226 | 91967128 | 097046 |
| 34318 | 92018650 | 92019149 | |
| 34319 | 92190822 | 92192000 | 069702 |
| 34320 | 92196202 | 92198133 | 069702 |
| 34321 | 92200616 | 92201379 | 069702 |
| 34322 | 92202102 | 92203018 | 069702 |
| 34323 | 92222946 | 92224290 | 069702 |
| 34324 | 92230778 | 92231202 | 069702 |
| 34325 | 92241596 | 92242253 | 069702 |
| 34326 | 92293071 | 92294051 | 069702 |
| 34327 | 92309923 | 92312051 | 069702 |
| 34328 | 92351112 | 92352239 | 069702 |
| 34329 | 92504277 | 92504820 | 172031 |
| 34330 | 92763740 | 92765398 | 122484, 174842 |
| 34331 | 92970452 | 92971515 | |
| 34332 | 93002262 | 93004262 | 067208 |
| 34333 | 93249413 | 93251285 | 067208 |
| 34334 | 93270160 | 93271296 | |
| 34335 | 93297225 | 93299020 | 122406 |
| 34336 | 93352199 | 93353117 | 154511 |
| 34337 | 93387299 | 93388889 | 154511 |
| 34338 | 93400296 | 93401811 | 154511 |
| 34339 | 93415881 | 93417889 | 154511 |
| 34340 | 93425399 | 93428446 | 154511 |
| 34341 | 93481575 | 93482185 | |
| 34342 | 93543813 | 93545921 | 143033 |
| 34343 | 93643699 | 93644653 | 117500 |
| 34344 | 93644931 | 93646506 | 122483, 117500 |
| 34345 | 93720559 | 93721337 | 122483 |
| 34346 | 93723090 | 93723696 | 122483 |
| 34347 | 93740465 | 93742199 | 122483, 223745 |
| 34348 | 93789591 | 93790798 | 223745 |
| 34349 | 93810386 | 93813177 | 117505, 223745 |
| 34350 | 93912636 | 93915228 | 137942 |
| 34351 | 93964931 | 93965526 | 137942 |
| 34352 | 94049845 | 94051533 | 137936 |
| 34353 | 94056979 | 94058928 | 224093, 137936 |
| 34354 | 94078819 | 94084481 | 137936 |
| 34355 | 94087453 | 94088608 | 137936 |
| 34356 | 94129807 | 94130820 | 137936 |
| 34357 | 94146201 | 94148207 | 137936 |
| 34358 | 94166133 | 94172495 | 137936 |
| 34359 | 94224016 | 94225461 | 137936, 230439 |
| 34360 | 94234299 | 94234936 | 137936, 230439 |
| 34361 | 94278323 | 94280047 | 137936 |
| 34362 | 94311144 | 94314165 | 211575, 260464, 137936 |
| 34363 | 94343866 | 94345790 | 067334 |
| 34364 | 94370812 | 94371796 | 023909 |
| 34365 | 94373267 | 94376299 | 023909 |
| 34366 | 94412118 | 94413273 | |
| 34367 | 94423725 | 94424246 | |
| 34368 | 94427852 | 94432643 | |
| 34369 | 94445221 | 94446535 | |
| 34370 | 94480108 | 94481661 | 198691 |
| 34371 | 94487932 | 94489097 | 198691 |
| 34372 | 94511014 | 94511737 | 198691 |
| 34373 | 94526721 | 94529321 | 198691 |
| 34374 | 94532966 | 94535478 | 198691 |
| 34375 | 94591366 | 94591917 | |
| 34376 | 94674747 | 94675628 | 137962 |
| 34377 | 94700955 | 94703520 | 137962 |
| 34378 | 94711866 | 94714141 | 231363, 137962 |
| 34379 | 94723642 | 94726934 | 137962 |
| 34380 | 94764298 | 94767164 | |
| 34381 | 94775296 | 94777182 | |
| 34382 | 94781097 | 94783752 | |
| 34383 | 94789734 | 94792569 | 236098 |
| 34384 | 94795352 | 94795961 | |
| 34385 | 94815357 | 94816376 | |
| 34386 | 94820805 | 94821695 | |
| 34387 | 94845057 | 94845717 | |
| 34388 | 94847753 | 94848114 | |
| 34389 | 94848911 | 94851513 | |
| 34390 | 94882946 | 94885262 | 117528 |
| 34391 | 94891191 | 94891996 | 117528 |
| 34392 | 94904096 | 94904955 | 117528 |
| 34393 | 95006559 | 95008310 | 117525 |
| 34394 | 95123321 | 95124378 | 224081 |
| 34395 | 95285257 | 95288002 | 143036, 224081 |
| 34396 | 95319767 | 95320841 | 237416, 143036 |
| 34397 | 95390188 | 95394365 | 235501, 117519 |
| 34398 | 95407185 | 95409811 | 235501 |
| 34399 | 95415209 | 95416402 | 235501 |
| 34400 | 95416672 | 95417610 | 235501 |
| 34401 | 95421245 | 95422182 | 235501 |
| 34402 | 95427118 | 95429224 | 235501 |
| 34403 | 95479350 | 95480035 | 172339 |
| 34404 | 95508777 | 95509636 | 172339 |
| 34405 | 95515345 | 95516028 | 172339 |
| 34406 | 95532570 | 95533124 | 230427, 172339 |
| 34407 | 95534148 | 95535410 | 172339 |
| 34408 | 95537385 | 95539434 | 172339 |
| 34409 | 95552213 | 95553850 | |
| 34410 | 95561206 | 95562542 | |
| 34411 | 95581265 | 95584196 | 271092, 152078 |
| 34412 | 95587361 | 95588369 | 231992, 271092, 152078 |
| 34413 | 95599074 | 95599870 | 231992, 271092, 152078 |
| 34414 | 95633259 | 95636050 | 271092, 152078, 226026 |
| 34415 | 95663176 | 95663930 | 271092, 226026 |
| 34416 | 95698078 | 95700651 | 122481, 226026, 271092 |
| 34417 | 95722668 | 95724109 | 228852 |
| 34418 | 96775358 | 96775773 | 231987 |
| 34419 | 96827415 | 96829638 | 231987 |
| 34420 | 96890819 | 96891487 | |
| 34421 | 96937779 | 96938445 | |
| 34422 | 97024840 | 97026941 | |
| 34423 | 97187002 | 97188519 | 117569 |
| 34424 | 97237825 | 97238387 | 117569 |
| 34425 | 97255767 | 97256802 | 117569 |
| 34426 | 97331129 | 97332083 | |
| 34427 | 97428439 | 97429082 | |
| 34428 | 97430205 | 97431109 | |
| 34429 | 97655833 | 97656301 | 232878, 188641 |
| 34430 | 97717548 | 97718195 | 232878, 188641 |
| 34431 | 97742696 | 97744596 | 232878, 188641 |
| 34432 | 97803225 | 97803519 | 188641 |
| 34433 | 97829791 | 97830941 | 188641 |
| 34434 | 97849889 | 97850162 | 188641 |
| 34435 | 97966246 | 97966934 | 188641 |
| 34436 | 97967685 | 97968440 | 188641 |
| 34437 | 98384496 | 98387121 | 188641 |
| 34438 | 98395448 | 98396625 | |
| 34439 | 98513747 | 98514259 | 225206 |
| 34440 | 98519044 | 98520272 | |
| 34441 | 98552204 | 98552778 | |
| 34442 | 98895382 | 98896058 | |
| 34443 | 99126519 | 99128413 | 162627 |
| 34444 | 99142726 | 99143476 | 162627 |
| 34445 | 99575487 | 99576864 | 232825 |
| 34446 | 99635702 | 99637437 | |
| 34447 | 100002971 | 100004999 | |
| 34448 | 100030279 | 100033189 | |
| 34449 | 100065139 | 100065685 | |
| 34450 | 100069042 | 100070094 | |
| 34451 | 100110640 | 100114303 | 099260 |
| 34452 | 100115377 | 100116594 | 099260 |
| 34453 | 100128093 | 100128587 | 099260 |
| 34454 | 100137919 | 100138858 | 099260 |
| 34455 | 100151089 | 100151774 | 099260 |
| 34456 | 100192381 | 100192918 | 156869 |
| 34457 | 100204057 | 100205267 | 156869 |
| 34458 | 100213305 | 100215476 | 156869 |
| 34459 | 100224785 | 100225985 | 156869 |
| 34460 | 100314798 | 100316531 | 162688 |
| 34461 | 100428375 | 100429418 | |
| 34462 | 100434608 | 100436367 | 117620, 228084 |
| 34463 | 100502376 | 100505229 | 156875 |
| 34464 | 100597938 | 100599346 | 122435, 156876 |
| 34465 | 100653391 | 100654485 | 137992 |
| 34466 | 100714545 | 100716083 | 230287, 137992 |
| 34467 | 100731242 | 100732844 | 137996, 224616 |
| 34468 | 100816428 | 100818918 | 079335 |
| 34469 | 100918206 | 100918745 | 079335 |
| 34470 | 100951137 | 100951927 | 228086, 079335 |
| 34471 | 100980681 | 100982048 | 079335 |
| 34472 | 101004458 | 101005846 | 181656 |
| 34473 | 101048451 | 101049000 | |
| 34474 | 101050436 | 101050983 | |
| 34475 | 101087520 | 101088640 | |
| 34476 | 101123192 | 101124910 | |
| 34477 | 101359907 | 101362608 | 273204, 162695, 162694 |
| 34478 | 101490392 | 101492514 | 233184, 117543 |
| 34479 | 101571300 | 101571899 | |
| 34480 | 101573243 | 101574138 | |
| 34481 | 101615166 | 101617273 | |
| 34482 | 101658737 | 101660246 | |
| 34483 | 101665988 | 101666556 | |
| 34484 | 101701018 | 101704764 | 170989, 225938 |
| 34485 | 101723104 | 101726859 | |
| 34486 | 101730039 | 101730664 | |
| 34487 | 101836708 | 101837132 | 231671 |
| 34488 | 104068111 | 104069472 | 185946 |
| 34489 | 109126793 | 109127461 | 162636 |
| 34490 | 109234033 | 109236331 | 134186 |
| 34491 | 109288826 | 109290384 | 116266 |
| 34492 | 109371680 | 109372830 | 162641 |
| 34493 | 109505161 | 109506383 | 121940, 162641 |
| 34494 | 109583859 | 109584896 | 085433 |
| 34495 | 109617668 | 109619335 | 197780 |
| 34496 | 109632647 | 109635043 | 273382, 215717 |
| 34497 | 109642425 | 109643856 | 270066 |
| 34498 | 109647393 | 109647814 | |
| 34499 | 109723721 | 109724696 | 116299 |
| 34500 | 109739760 | 109741377 | 116299 |
| 34501 | 109756130 | 109758527 | 031698 |
| 34502 | 109806038 | 109806867 | 143126 |
| 34503 | 109816459 | 109819013 | 143126 |
| 34504 | 109825780 | 109826854 | 134222 |
| 34505 | 109913186 | 109914351 | 134243 |
| 34506 | 109934979 | 109936261 | 134243 |
| 34507 | 109939781 | 109940724 | 134243 |
| 34508 | 109967423 | 109970257 | 143106 |
| 34509 | 109980670 | 109981996 | |
| 34510 | 110008787 | 110010144 | 143028 |
| 34511 | 110026295 | 110027259 | 162650 |
| 34512 | 110036340 | 110037335 | 174151 |
| 34513 | 110051839 | 110052593 | 181754 |
| 34514 | 110088798 | 110091951 | 065135, 156097 |
| 34515 | 110161174 | 110163936 | 116337 |
| 34516 | 110171002 | 110171472 | 228703, 116337 |
| 34517 | 110185749 | 110186695 | |
| 34518 | 110198093 | 110199362 | 168765 |
| 34519 | 110306281 | 110306993 | 198758, 241720, 134201 |
| 34520 | 110366286 | 110367157 | |
| 34521 | 110415882 | 110416686 | |
| 34522 | 110419414 | 110420634 | |
| 34523 | 110435018 | 110435560 | |
| 34524 | 110438355 | 110438871 | 261055 |
| 34525 | 110443417 | 110444893 | |
| 34526 | 110446208 | 110447499 | |
| 34527 | 110452538 | 110456548 | 184371 |
| 34528 | 110459240 | 110461846 | 184371 |
| 34529 | 110468922 | 110472703 | 184371 |
| 34530 | 110473493 | 110474821 | 184371 |
| 34531 | 110476009 | 110479206 | |
| 34532 | 110490299 | 110491352 | |
| 34533 | 110501773 | 110503583 | |
| 34534 | 110503888 | 110504583 | |
| 34535 | 110518082 | 110519342 | |
| 34536 | 110520855 | 110522264 | |
| 34537 | 110526017 | 110528685 | 168710 |
| 34538 | 110545923 | 110547525 | 168710 |
| 34539 | 110576418 | 110578447 | 143093 |
| 34540 | 110591701 | 110592283 | 143093 |
| 34541 | 110814684 | 110815172 | 200536, 116396 |
| 34542 | 110878945 | 110883286 | 162775, 227963 |
| 34543 | 110898940 | 110900163 | |
| 34544 | 110935651 | 110937647 | 224699 |
| 34545 | 110949438 | 110951322 | 134248, 224699 |
| 34546 | 110976405 | 110977367 | |
| 34547 | 111019516 | 111020896 | |
| 34548 | 111505558 | 111507260 | 273010, 121931 |
| 34549 | 111587746 | 111588270 | |
| 34550 | 111681775 | 111683922 | 134255, 156171 |
| 34551 | 111684257 | 111685599 | 134255 |
| 34552 | 111733428 | 111734427 | 162777 |
| 34553 | 111735284 | 111737046 | 162777 |
| 34554 | 111745357 | 111747665 | 162777, 064886 |
| 34555 | 111770983 | 111771409 | 064886 |
| 34556 | 111888638 | 111889202 | 173947 |
| 34557 | 111906459 | 111908703 | 234020 |
| 34558 | 111948675 | 111949230 | |
| 34559 | 111960193 | 111960815 | 085465 |
| 34560 | 111965456 | 111966079 | 085465 |
| 34561 | 111990795 | 111993163 | 116459, 116455 |
| 34562 | 112050145 | 112052711 | 121933 |
| 34563 | 112077720 | 112079134 | 121933 |
| 34564 | 112081036 | 112081785 | 121933 |
| 34565 | 112084051 | 112085211 | 116473, 121933 |
| 34566 | 112102313 | 112102845 | 121933, 116473 |
| 34567 | 112103971 | 112104462 | 121933, 116473 |
| 34568 | 112106152 | 112107269 | 121933, 116473 |
| 34569 | 112135234 | 112136527 | 116473 |
| 34570 | 112161728 | 112163841 | 116473 |
| 34571 | 112246904 | 112247808 | 197852, 116473 |
| 34572 | 112280976 | 112282822 | 227811, 197852 |
| 34573 | 112297468 | 112299590 | 064703, 197852 |
| 34574 | 112405047 | 112406116 | 171385 |
| 34575 | 112420268 | 112423314 | 171385 |
| 34576 | 112423616 | 112424570 | 171385 |
| 34577 | 112429264 | 112431018 | 171385 |
| 34578 | 112518128 | 112520254 | 171385 |
| 34579 | 112530978 | 112534470 | 231437, 171385 |
| 34580 | 112902883 | 112903318 | 231246 |
| 34581 | 112933386 | 112934218 | |
| 34582 | 112937967 | 112939621 | 143079 |
| 34583 | 113086086 | 113087918 | 007341 |
| 34584 | 113160654 | 113163612 | 116489, 007341 |
| 34585 | 113216107 | 113218589 | 155363 |
| 34586 | 113240953 | 113242191 | 155363 |
| 34587 | 113242373 | 113244451 | 155366, 155363 |
| 34588 | 113246640 | 113250420 | 155366, 271810 |
| 34589 | 113257454 | 113258253 | 155367 |
| 34590 | 113477475 | 113480119 | 155380 |
| 34591 | 113497450 | 113499664 | 226419, 155380 |
| 34592 | 113536680 | 113537899 | 226419 |
| 34593 | 113569607 | 113570505 | 238198, 236066 |
| 34594 | 113572061 | 113572846 | 238198, 236066 |
| 34595 | 113574002 | 113574450 | 238198, 236066 |
| 34596 | 113614618 | 113616908 | 198799, 238198 |
| 34597 | 113797821 | 113798479 | |
| 34598 | 113834716 | 113835692 | |
| 34599 | 113930859 | 113934754 | 081026 |
| 34600 | 113936282 | 113937737 | 081026 |
| 34601 | 113998820 | 114001116 | 081026 |
| 34602 | 114022668 | 114023402 | 081026 |
| 34603 | 114076639 | 114077316 | 081026 |
| 34604 | 114217138 | 114217747 | 081026 |
| 34605 | 114219710 | 114220993 | 081026 |
| 34606 | 114300754 | 114303034 | 116793 |
| 34607 | 114347302 | 114348018 | 081019 |
| 34608 | 114353713 | 114355477 | 231128, 081019 |
| 34609 | 114446635 | 114448660 | 118655, 134262 |
| 34610 | 114470772 | 114473585 | 163349, 235527 |
| 34611 | 114888397 | 114889713 | |
| 34612 | 114977245 | 114978403 | 197323 |
| 34613 | 114995900 | 114996641 | 197323 |
| 34614 | 115052616 | 115054945 | 197323 |
| 34615 | 115123400 | 115125180 | 116752 |
| 34616 | 115129820 | 115132098 | 175984 |
| 34617 | 115211536 | 115213850 | 175984 |
| 34618 | 115258441 | 115260196 | 009307, 213281 |
| 34619 | 115262876 | 115263818 | 009307 |
| 34620 | 115298911 | 115301696 | 009307 |
| 34621 | 115322441 | 115323634 | 052723 |
| 34622 | 115676385 | 115680384 | |
| 34623 | 115681354 | 115682039 | |
| 34624 | 115710823 | 115712999 | |
| 34625 | 116087759 | 116089602 | |
| 34626 | 116184251 | 116185612 | 173218 |
| 34627 | 116270347 | 116270866 | 118729 |
| 34628 | 116423777 | 116424380 | |
| 34629 | 116518482 | 116521214 | 163393 |
| 34630 | 116652074 | 116654778 | 173212 |
| 34631 | 116665322 | 116667247 | 173212 |
| 34632 | 116681263 | 116682217 | |
| 34633 | 116694522 | 116695189 | |
| 34634 | 116695440 | 116696742 | |
| 34635 | 116721511 | 116722713 | |
| 34636 | 116810885 | 116811292 | |
| 34637 | 116815681 | 116816597 | |
| 34638 | 116831731 | 116832407 | |
| 34639 | 116876501 | 116877696 | |
| 34640 | 116914951 | 116917087 | 269279, 163399 |
| 34641 | 116917995 | 116923944 | 269279, 163399 |
| 34642 | 116931444 | 116932116 | 163399 |
| 34643 | 116960290 | 116961924 | 203865 |
| 34644 | 117031472 | 117032889 | |
| 34645 | 117035918 | 117039638 | 224950 |
| 34646 | 117046540 | 117047567 | |
| 34647 | 117051193 | 117052718 | |
| 34648 | 117080987 | 117081731 | 116815 |
| 34649 | 117112517 | 117114125 | 116815 |
| 34650 | 117190935 | 117191371 | 143061 |
| 34651 | 117209535 | 117210732 | 143061 |
| 34652 | 117229696 | 117230751 | |
| 34653 | 117320369 | 117321352 | |
| 34654 | 117396172 | 117397257 | |
| 34655 | 117452212 | 117453783 | 134247 |
| 34656 | 117463501 | 117464842 | 134247 |
| 34657 | 117539389 | 117540386 | |
| 34658 | 117602815 | 117603799 | 116830 |
| 34659 | 117704492 | 117705308 | 134258 |
| 34660 | 117714200 | 117715064 | 134258 |
| 34661 | 117908501 | 117911409 | 198162 |
| 34662 | 118087032 | 118088106 | |
| 34663 | 118134779 | 118135297 | |
| 34664 | 118147949 | 118149848 | 183508, 236866 |
| 34665 | 118172549 | 118173598 | |
| 34666 | 118277576 | 118278659 | |
| 34667 | 118301170 | 118301968 | |
| 34668 | 118360273 | 118360998 | |
| 34669 | 118368308 | 118369856 | |
| 34670 | 118389612 | 118390478 | |
| 34671 | 118471528 | 118473590 | 065183, 196505 |
| 34672 | 118653936 | 118654478 | 155761 |
| 34673 | 118659171 | 118659947 | 155761 |
| 34674 | 118660700 | 118661940 | 155761 |
| 34675 | 119357788 | 119359086 | |
| 34676 | 119455086 | 119458036 | 092607 |
| 34677 | 119502285 | 119507736 | 092607 |
| 34678 | 119682413 | 119684050 | 231365, 116874 |
| 34679 | 119808916 | 119809447 | 227712, 231365 |
| 34680 | 119821809 | 119823143 | 227712 |
| 34681 | 119868884 | 119871007 | 232650, 227712 |
| 34682 | 119883084 | 119884944 | 232650 |
| 34683 | 119915038 | 119918045 | 116882 |
| 34684 | 119921443 | 119924564 | 116882 |
| 34685 | 120081765 | 120086662 | 249798 |
| 34686 | 120173422 | 120174981 | 143067 |
| 34687 | 120187784 | 120191829 | 143067 |
| 34688 | 120217365 | 120219613 | 092621 |
| 34689 | 120254471 | 120256584 | 092621 |
| 34690 | 120271555 | 120272213 | 092621 |
| 34691 | 120308179 | 120309804 | 134240 |
| 34692 | 120311291 | 120311916 | 134240 |
| 34693 | 120316177 | 120316954 | |
| 34694 | 120321872 | 120323365 | |
| 34695 | 120326670 | 120327526 | |
| 34696 | 120332813 | 120333879 | |
| 34697 | 120358582 | 120359870 | |
| 34698 | 120435827 | 120438076 | 134249 |
| 34699 | 120445576 | 120446737 | |
| 34700 | 120449306 | 120450389 | |
| 34701 | 120500524 | 120501336 | 134250 |
| 34702 | 120516438 | 120517062 | 134250 |
| 34703 | 120529045 | 120531186 | 134250 |
| 34704 | 121252978 | 121253816 | |
| 34705 | 121260449 | 121261746 | 231752, 272583 |
| 34706 | 121263474 | 121264295 | 231752 |
| 34707 | 121484147 | 121485604 | |
| 34708 | 144009590 | 144011508 | 196369 |
| 34709 | 144533439 | 144534491 | 201699 |
| 34710 | 144921448 | 144922181 | 178104 |
| 34711 | 144930257 | 144933431 | 178104 |
| 34712 | 144987908 | 144990573 | 268172, 178104 |
| 34713 | 145039048 | 145040193 | 178104 |
| 34714 | 145042645 | 145043285 | 178104 |
| 34715 | 145096033 | 145097470 | 223380 |
| 34716 | 145113361 | 145115560 | 223380 |
| 34717 | 145382146 | 145383564 | 201558, 233396 |
| 34718 | 145390014 | 145391215 | |
| 34719 | 145396492 | 145398218 | |
| 34720 | 145412767 | 145415819 | 168509 |
| 34721 | 145434994 | 145435730 | |
| 34722 | 145437412 | 145439452 | 117289 |
| 34723 | 145448432 | 145449253 | |
| 34724 | 145462105 | 145462620 | 121851 |
| 34725 | 145469883 | 145471430 | 181039, 121851 |
| 34726 | 145473701 | 145474629 | 181039 |
| 34727 | 145476852 | 145478358 | 152022, 234222 |
| 34728 | 145506731 | 145508704 | 131795, 234222 |
| 34729 | 145515080 | 145517378 | 131779, 211451 |
| 34730 | 145541892 | 145543925 | 244619, 143127 |
| 34731 | 145575103 | 145576674 | 131788 |
| 34732 | 145588444 | 145589843 | 186364 |
| 34733 | 145609985 | 145611642 | 121848, 186141 |
| 34734 | 145649109 | 145654057 | 121848 |
| 34735 | 145668933 | 145669780 | 121848 |
| 34736 | 145703988 | 145705204 | 117281 |
| 34737 | 145713137 | 145714600 | 117281 |
| 34738 | 145719772 | 145720801 | |
| 34739 | 145722886 | 145724054 | |
| 34740 | 145726481 | 145729712 | 174827 |
| 34741 | 145742271 | 145745724 | 174827 |
| 34742 | 146555569 | 146557394 | 227242 |
| 34743 | 146581278 | 146581897 | 227242 |
| 34744 | 146582395 | 146583116 | 227242 |
| 34745 | 146637961 | 146645595 | 273071, 237188, 131791 |
| 34746 | 146696344 | 146697476 | 273071, 131781 |
| 34747 | 146708803 | 146710455 | 273071, 131781 |
| 34748 | 146713147 | 146715431 | 131778, 131781, 273071 |
| 34749 | 146744971 | 146745826 | 131778 |
| 34750 | 146763205 | 146765887 | 131778 |
| 34751 | 146944333 | 146945511 | |
| 34752 | 146951315 | 146952684 | |
| 34753 | 146986828 | 146988084 | 234610 |
| 34754 | 147010216 | 147011388 | |
| 34755 | 147011875 | 147013925 | 116128 |
| 34756 | 147070862 | 147072861 | 116128 |
| 34757 | 147141252 | 147143055 | 162836 |
| 34758 | 147280079 | 147280691 | |
| 34759 | 147322930 | 147323862 | |
| 34760 | 147735471 | 147736325 | 199879 |
| 34761 | 147806323 | 147807486 | 202408, 224481 |
| 34762 | 148241254 | 148241864 | 207501 |
| 34763 | 149214772 | 149215898 | 252826 |
| 34764 | 149222762 | 149225490 | 206737 |
| 34765 | 149230178 | 149231488 | 232721 |
| 34766 | 149235393 | 149235937 | |
| 34767 | 149856459 | 149860750 | 178096, 184270, 184260, |
| | | | 184678 |
| 34768 | 149870866 | 149872259 | 178096 |
| 34769 | 149898948 | 149900818 | 014914, 143368 |
| 34770 | 149910720 | 149911982 | 163113 |
| 34771 | 149950265 | 149950976 | 163113 |
| 34772 | 149953009 | 149953385 | 163113 |
| 34773 | 149967875 | 149968493 | 163113 |
| 34774 | 149979639 | 149983507 | 163113 |
| 34775 | 149992420 | 149994850 | |
| 34776 | 150038421 | 150040513 | 136631 |
| 34777 | 150121423 | 150123120 | 023902 |
| 34778 | 150145162 | 150146210 | |
| 34779 | 150157732 | 150160036 | |
| 34780 | 150163840 | 150165016 | |
| 34781 | 150179515 | 150180082 | |
| 34782 | 150185526 | 150186380 | |
| 34783 | 150206605 | 150208743 | 143401 |
| 34784 | 150227682 | 150229838 | 118298, 271845 |
| 34785 | 150239979 | 150242206 | 118292, 117362 |
| 34786 | 150250599 | 150251189 | 118292 |
| 34787 | 150252298 | 150255225 | 159208, 118292 |
| 34788 | 150265566 | 150267132 | 187145 |
| 34789 | 150293026 | 150295087 | 117360 |
| 34790 | 150335341 | 150339421 | 163125 |
| 34791 | 150359601 | 150360226 | 163125 |
| 34792 | 150381874 | 150383832 | 163125 |
| 34793 | 150445936 | 150446701 | 163125 |
| 34794 | 150458952 | 150460683 | 143374 |
| 34795 | 150487451 | 150488657 | 228126 |
| 34796 | 150504412 | 150505670 | |
| 34797 | 150508453 | 150508961 | |
| 34798 | 150516111 | 150517368 | |
| 34799 | 150520887 | 150522954 | 225996, 143382, 237781 |
| 34800 | 150532206 | 150537516 | 203804, 143382 |
| 34801 | 150539288 | 150541783 | 264584, 264508 |
| 34802 | 150546592 | 150553239 | 143384 |
| 34803 | 150573225 | 150573698 | 143420 |
| 34804 | 150575493 | 150578408 | 143420 |
| 34805 | 150583622 | 150587468 | 143420 |
| 34806 | 150593897 | 150594453 | 143420 |
| 34807 | 150600624 | 150603080 | 143420 |
| 34808 | 150669206 | 150670538 | 143452, 143457 |
| 34809 | 150736980 | 150738648 | 163131 |
| 34810 | 150793160 | 150793645 | 143437 |
| 34811 | 150824637 | 150825188 | 143437 |
| 34812 | 150847126 | 150850266 | 143437 |
| 34813 | 150853871 | 150854807 | 224800 |
| 34814 | 150894860 | 150895486 | |
| 34815 | 150898069 | 150899751 | 143379 |
| 34816 | 150943178 | 150948252 | 231073, 143418 |
| 34817 | 150949217 | 150950715 | |
| 34818 | 150951313 | 150952451 | |
| 34819 | 150953416 | 150955063 | 143412 |
| 34820 | 150957642 | 150958127 | 143412 |
| 34821 | 150980298 | 150981941 | 143363, 143409 |
| 34822 | 150985195 | 150986262 | 143363 |
| 34823 | 151019976 | 151021642 | 143443, 163141 |
| 34824 | 151030855 | 151033477 | 197622, 213190 |
| 34825 | 151035106 | 151036127 | 197622, 213190 |
| 34826 | 151042371 | 151044298 | 143458, 197622 |
| 34827 | 151137215 | 151139510 | 163155, 163156 |
| 34828 | 151161474 | 151163319 | 163159 |
| 34829 | 151167594 | 151169249 | 163159 |
| 34830 | 151169802 | 151173100 | 143398 |
| 34831 | 151226646 | 151228365 | 159352 |
| 34832 | 151237300 | 151239516 | 159352 |
| 34833 | 151246165 | 151246982 | |
| 34834 | 151253488 | 151256172 | 143373, 232671 |
| 34835 | 151281467 | 151285468 | 143393 |
| 34836 | 151285707 | 151287291 | 143393 |
| 34837 | 151298807 | 151301335 | 273481, 143393 |
| 34838 | 151318964 | 151320335 | 237976, 143390 |
| 34839 | 151340554 | 151341551 | 143416 |
| 34840 | 151343711 | 151344493 | 143416 |
| 34841 | 151344536 | 151345430 | 143416 |
| 34842 | 151371458 | 151373221 | 159377 |
| 34843 | 151430380 | 151432747 | 143442 |
| 34844 | 151445299 | 151446608 | |
| 34845 | 151466797 | 151468266 | |
| 34846 | 151481989 | 151483175 | 143375 |
| 34847 | 151483445 | 151487459 | 143375 |
| 34848 | 151488488 | 151489194 | 143375 |
| 34849 | 151511416 | 151514284 | 232536, 143367 |
| 34850 | 151518772 | 151521773 | 232536, 143367 |
| 34851 | 151540868 | 151543339 | 143367 |
| 34852 | 151568376 | 151570365 | |
| 34853 | 151578959 | 151579775 | |
| 34854 | 151580842 | 151581392 | |
| 34855 | 151583638 | 151585478 | 250734, 143376 |
| 34856 | 151611100 | 151612329 | 143376 |
| 34857 | 151734981 | 151737015 | 249602, 143450, 143436 |
| 34858 | 151762438 | 151764301 | 203288, 182134 |
| 34859 | 151791426 | 151793801 | 143365 |
| 34860 | 151803011 | 151804682 | 143365 |
| 34861 | 151804890 | 151805586 | |
| 34862 | 151810314 | 151811971 | 225556 |
| 34863 | 151859260 | 151859871 | 159445 |
| 34864 | 151881171 | 151882994 | 159445 |
| 34865 | 151891054 | 151892152 | |
| 34866 | 151913184 | 151914207 | |
| 34867 | 151918178 | 151919223 | |
| 34868 | 151960241 | 151967952 | 229021, 197747 |
| 34869 | 151970406 | 151972509 | 229021 |
| 34870 | 151973923 | 151974682 | 229021 |
| 34871 | 152008410 | 152009846 | 163191, 229021 |
| 34872 | 152019544 | 152021601 | 163191 |
| 34873 | 152161005 | 152162929 | 237975 |
| 34874 | 152489325 | 152490903 | |
| 34875 | 152901043 | 152901653 | |
| 34876 | 153329981 | 153330514 | 163220 |
| 34877 | 153462772 | 153463205 | |
| 34878 | 153478689 | 153480442 | |
| 34879 | 153506290 | 153509204 | 197956, 238279 |
| 34880 | 153521576 | 153522347 | 188015, 196154 |
| 34881 | 153537577 | 153542044 | 196754 |
| 34882 | 153579753 | 153586406 | 188643 |
| 34883 | 153588437 | 153590503 | 189334 |
| 34884 | 153605831 | 153607498 | 160679, 271853, 189171, |
| | | | 272030 |
| 34885 | 153630619 | 153631890 | 143553 |
| 34886 | 153642596 | 153644707 | 143621 |
| 34887 | 153671605 | 153672129 | |
| 34888 | 153699652 | 153701589 | 143624 |
| 34889 | 153713394 | 153716699 | 143624 |
| 34890 | 153740563 | 153742102 | 143624 |
| 34891 | 153747549 | 153748647 | 143554 |
| 34892 | 153749751 | 153750461 | 143554 |
| 34893 | 153752186 | 153753023 | 143554 |
| 34894 | 153755385 | 153756606 | |
| 34895 | 153868658 | 153869480 | 143614 |
| 34896 | 153894202 | 153896906 | 143614 |
| 34897 | 153917772 | 153919673 | 198837 |
| 34898 | 153929908 | 153931286 | 160741 |
| 34899 | 153934531 | 153937168 | 143570 |
| 34900 | 153939092 | 153940594 | 143578, 273026, 143570 |
| 34901 | 153948997 | 153951221 | 272654, 143543 |
| 34902 | 153962693 | 153964537 | 177954 |
| 34903 | 153978636 | 153979036 | 143552 |
| 34904 | 154154399 | 154156220 | 143549 |
| 34905 | 154191871 | 154194784 | 143569, 143612 |
| 34906 | 154209471 | 154210870 | 143569 |
| 34907 | 154244406 | 154245870 | 143575 |
| 34908 | 154306502 | 154307985 | 143515 |
| 34909 | 154324525 | 154326512 | |
| 34910 | 154376532 | 154383565 | 160712, 228013 |
| 34911 | 154388849 | 154397310 | 160712 |
| 34912 | 154399260 | 154401607 | 160712 |
| 34913 | 154411918 | 154412964 | 160712 |
| 34914 | 154425862 | 154427049 | 160712 |
| 34915 | 154473924 | 154474839 | 163239, 169291 |
| 34916 | 154520407 | 154521197 | 160714, 163239 |
| 34917 | 154530267 | 154532513 | 160714 |
| 34918 | 154579387 | 154581549 | 160710 |
| 34919 | 154599113 | 154601142 | 160710 |
| 34920 | 154670261 | 154671599 | 143603 |
| 34921 | 154757292 | 154758021 | 143603 |
| 34922 | 154849140 | 154851474 | |
| 34923 | 154903964 | 154905310 | 163344 |
| 34924 | 154908309 | 154910859 | 270361, 163344 |
| 34925 | 154917158 | 154917689 | 163346 |
| 34926 | 154933453 | 154935329 | 160691, 271380, 163348 |
| 34927 | 154942999 | 154949064 | 264349, 173207, 160691 |
| 34928 | 154955516 | 154956884 | 160688 |
| 34929 | 154971594 | 154979316 | 160685 |
| 34930 | 154989096 | 154990446 | 160685 |
| 34931 | 154997600 | 154998031 | 163354 |
| 34932 | 154999032 | 154999548 | 163354 |
| 34933 | 155016983 | 155017779 | 232093, 163357 |
| 34934 | 155022149 | 155024983 | 143537, 163357 |
| 34935 | 155035895 | 155036682 | 251246, 143590, 243364 |
| 34936 | 155056531 | 155057706 | 251246, 143590 |
| 34937 | 155058044 | 155058970 | 251246, 143590 |
| 34938 | 155077895 | 155078981 | |
| 34939 | 155079059 | 155081398 | |
| 34940 | 155096809 | 155104848 | 169242 |
| 34941 | 155105619 | 155109122 | 169241, 169242 |
| 34942 | 155111826 | 155113390 | 179085 |
| 34943 | 155144783 | 155146066 | 163462, 163463, 273088 |
| 34944 | 155162834 | 155163790 | |
| 34945 | 155177723 | 155179769 | 173171, 169231 |
| 34946 | 155194368 | 155195058 | 160766 |
| 34947 | 155196178 | 155197375 | 160766 |
| 34948 | 155213528 | 155214940 | 177628 |
| 34949 | 155224426 | 155226054 | 116521, 160767 |
| 34950 | 155230836 | 155232934 | 176444, 116521 |
| 34951 | 155242363 | 155243717 | 176444 |
| 34952 | 155246987 | 155248546 | 143630, 176444 |
| 34953 | 155269795 | 155271361 | 143627 |
| 34954 | 155277747 | 155279923 | 160752 |
| 34955 | 155292676 | 155295293 | 225855, 160753 |
| 34956 | 155435684 | 155437247 | 116539 |
| 34957 | 155468867 | 155470032 | 116539 |
| 34958 | 155531684 | 155534073 | 235919, 116539 |
| 34959 | 155618303 | 155618926 | 246203, 125459, 203761 |
| 34960 | 155657606 | 155659497 | 132676, 163374, 125459, |
| | | | 203761 |
| 34961 | 155668109 | 155668731 | 132676, 125459, 203761 |
| 34962 | 155826151 | 155827340 | 116580 |
| 34963 | 155879980 | 155881456 | 143622 |
| 34964 | 155903169 | 155905223 | 132680 |
| 34965 | 155947256 | 155949235 | 273002, 116584 |
| 34966 | 155951289 | 155954718 | 273002, 116584 |
| 34967 | 155958576 | 155961941 | 224276, 116584 |
| 34968 | 155989841 | 155991142 | 163479 |
| 34969 | 156022768 | 156025496 | 116586, 160803 |
| 34970 | 156039369 | 156040853 | 132698 |
| 34971 | 156045653 | 156047183 | 222611, 254726 |
| 34972 | 156051724 | 156053314 | 160789, 254726 |
| 34973 | 156055792 | 156056553 | 160789 |
| 34974 | 156075677 | 156077191 | 160789 |
| 34975 | 156083069 | 156086575 | 160789 |
| 34976 | 156088085 | 156088745 | 160789 |
| 34977 | 156091800 | 156094500 | 160789 |
| 34978 | 156098894 | 156100679 | 160789 |
| 34979 | 156134755 | 156135679 | 196189 |
| 34980 | 156153708 | 156154401 | |
| 34981 | 156162830 | 156164929 | 160785 |
| 34982 | 156182365 | 156184155 | 260238, 160783, 160785 |
| 34983 | 156186117 | 156186767 | 260238, 160783 |
| 34984 | 156187594 | 156188315 | 260238, 160783 |
| 34985 | 156232081 | 156233728 | 198952 |
| 34986 | 156246822 | 156247216 | 198952 |
| 34987 | 156251526 | 156253424 | 163472, 198952 |
| 34988 | 156260838 | 156262433 | 198715, 163472 |
| 34989 | 156264593 | 156265790 | 198715 |
| 34990 | 156271336 | 156272076 | |
| 34991 | 156307124 | 156309212 | 163468, 163467 |
| 34992 | 156363793 | 156364299 | 237390 |
| 34993 | 156425705 | 156428443 | 236947 |
| 34994 | 156441216 | 156441903 | 116604 |
| 34995 | 156465449 | 156468033 | 116604 |
| 34996 | 156468207 | 156471943 | 116604 |
| 34997 | 156472230 | 156475879 | |
| 34998 | 156560978 | 156562417 | 163382 |
| 34999 | 156570411 | 156572061 | 160818 |
| 35000 | 156629684 | 156631612 | 229953 |
| 35001 | 156647636 | 156648241 | |
| 35002 | 156697258 | 156699440 | 143303, 143319 |
| 35003 | 156709689 | 156711679 | 143314 |
| 35004 | 156715747 | 156722609 | 143294, 143321 |
| 35005 | 156736201 | 156738476 | 143321, 143294 |
| 35006 | 156821835 | 156822782 | 198400, 027644 |
| 35007 | 156882679 | 156883101 | 187800 |
| 35008 | 157014647 | 157016580 | 132694 |
| 35009 | 157107141 | 157109495 | 117036 |
| 35010 | 157124501 | 157125247 | |
| 35011 | 157210080 | 157211141 | |
| 35012 | 157281939 | 157282529 | |
| 35013 | 157282880 | 157283641 | |
| 35014 | 157409048 | 157409683 | |
| 35015 | 157411454 | 157412563 | |
| 35016 | 157931610 | 157933434 | |
| 35017 | 157934736 | 157936964 | |
| 35018 | 157989025 | 157990188 | 183853 |
| 35019 | 158118972 | 158119891 | |
| 35020 | 158147533 | 158148355 | 227295 |
| 35021 | 158150248 | 158151424 | 158473 |
| 35022 | 158155479 | 158156324 | |
| 35023 | 158496896 | 158497467 | |
| 35024 | 158969273 | 158970576 | 163565 |
| 35025 | 158979235 | 158980664 | 163565 |
| 35026 | 159198643 | 159199940 | |
| 35027 | 159251724 | 159253103 | |
| 35028 | 159411887 | 159413544 | 228560 |
| 35029 | 159478420 | 159481901 | |
| 35030 | 159526226 | 159527606 | 231100 |
| 35031 | 159528463 | 159528983 | |
| 35032 | 159550384 | 159551754 | 271890 |
| 35033 | 159557039 | 159561531 | 132703 |
| 35034 | 159566240 | 159569468 | 158731 |
| 35035 | 159674972 | 159676518 | 223603 |
| 35036 | 159681287 | 159684912 | 132693 |
| 35037 | 159721199 | 159723966 | |
| 35038 | 159725360 | 159726742 | |
| 35039 | 159749983 | 159751731 | 158716 |
| 35040 | 159885895 | 159886721 | |
| 35041 | 159891312 | 159896739 | 158710 |
| 35042 | 159927724 | 159928218 | |
| 35043 | 159940397 | 159941691 | 224259 |
| 35044 | 159961148 | 159962130 | |
| 35045 | 160000679 | 160002176 | 143315 |
| 35046 | 160063684 | 160064365 | 162729 |
| 35047 | 160067427 | 160069605 | 162729 |
| 35048 | 160093783 | 160095936 | 018625 |
| 35049 | 160174052 | 160174653 | 227741 |
| 35050 | 160174856 | 160176613 | 162734, 265381, 227741 |
| 35051 | 160216735 | 160217379 | 132716, 258465 |
| 35052 | 160230895 | 160233479 | 228606, 132716, 258465 |
| 35053 | 160253719 | 160255255 | 132716, 258465, 162735 |
| 35054 | 160309905 | 160310289 | 122218 |
| 35055 | 160312157 | 160314196 | 162736, 122218 |
| 35056 | 160508411 | 160511118 | 066294, 234425 |
| 35057 | 160607498 | 160608322 | 117090 |
| 35058 | 160608536 | 160609265 | 117090 |
| 35059 | 160931051 | 160931601 | |
| 35060 | 160959524 | 160960539 | |
| 35061 | 160978659 | 160979289 | 158769, 270149 |
| 35062 | 160982650 | 160984667 | 158769, 270149 |
| 35063 | 160989632 | 160992442 | 158769, 270149 |
| 35064 | 161008282 | 161008886 | 215845, 270149 |
| 35065 | 161014628 | 161016712 | 158773 |
| 35066 | 161052479 | 161054158 | 143217 |
| 35067 | 161067211 | 161068956 | 162755 |
| 35068 | 161086674 | 161089943 | 158793, 143256 |
| 35069 | 161101416 | 161103747 | 158796 |
| 35070 | 161123184 | 161124560 | 224985, 143222 |
| 35071 | 161128663 | 161130182 | 143258, 224985 |
| 35072 | 161135593 | 161137152 | 143224 |
| 35073 | 161146412 | 161148307 | 158850, 143224 |
| 35074 | 161162252 | 161164380 | 158859 |
| 35075 | 161170558 | 161173455 | 158864 |
| 35076 | 161186025 | 161190832 | 268387, 158869 |
| 35077 | 161191705 | 161198525 | 263548, 158882, 158874, |
| | | | 268387 |
| 35078 | 161207376 | 161208456 | 143257 |
| 35079 | 161214518 | 161215213 | |
| 35080 | 161283264 | 161285496 | 143252 |
| 35081 | 161351752 | 161353170 | |
| 35082 | 161358731 | 161360344 | |
| 35083 | 161368426 | 161370733 | |
| 35084 | 161409714 | 161410802 | |
| 35085 | 161500580 | 161501461 | 273112 |
| 35086 | 161509785 | 161510412 | 273112 |
| 35087 | 161646568 | 161647188 | 072694 |
| 35088 | 161675752 | 161677028 | 132185 |
| 35089 | 161695688 | 161697882 | 162746 |
| 35090 | 161719177 | 161720315 | 081721 |
| 35091 | 161734954 | 161737302 | 226889, 118217 |
| 35092 | 161785384 | 161786550 | 118217 |
| 35093 | 161879922 | 161880820 | 118217 |
| 35094 | 161882668 | 161885263 | 118217 |
| 35095 | 161993513 | 161994565 | 162745 |
| 35096 | 162038835 | 162041070 | 198929 |
| 35097 | 162044092 | 162044768 | 198929 |
| 35098 | 162050580 | 162051538 | 198929 |
| 35099 | 162061761 | 162062525 | 198929 |
| 35100 | 162101025 | 162102265 | 198929 |
| 35101 | 162106029 | 162107939 | 198929 |
| 35102 | 162110750 | 162112461 | 198929 |
| 35103 | 162121987 | 162122987 | 198929 |
| 35104 | 162161296 | 162161712 | 198929 |
| 35105 | 162164175 | 162164811 | 198929 |
| 35106 | 162166168 | 162167866 | 198929 |
| 35107 | 162176912 | 162177469 | 198929 |
| 35108 | 162183024 | 162183768 | 198929 |
| 35109 | 162204190 | 162205787 | 198929 |
| 35110 | 162210231 | 162210784 | 198929 |
| 35111 | 162217007 | 162217559 | 198929 |
| 35112 | 162262010 | 162262791 | 198929 |
| 35113 | 162275736 | 162276223 | 198929 |
| 35114 | 162330001 | 162331713 | 198929 |
| 35115 | 162333399 | 162337075 | 239887, 254706, 198929 |
| 35116 | 162340673 | 162345339 | 171722, 198929, 239887, |
| | | | 254706 |
| 35117 | 162350156 | 162352887 | 254706, 198929, 239887 |
| 35118 | 162357762 | 162360111 | |
| 35119 | 162467194 | 162468460 | 152332 |
| 35120 | 162530236 | 162532816 | 117143, 259788 |
| 35121 | 162566337 | 162567256 | 117143 |
| 35122 | 162582290 | 162583056 | |
| 35123 | 162647872 | 162648972 | 162733 |
| 35124 | 162650791 | 162652492 | 162733 |
| 35125 | 162656895 | 162658624 | 162733 |
| 35126 | 162710302 | 162711207 | 162733 |
| 35127 | 163290632 | 163292238 | 143248, 232995, 143228 |
| 35128 | 164604833 | 164607367 | 185630 |
| 35129 | 164717027 | 164718609 | 185630 |
| 35130 | 165121310 | 165121967 | |
| 35131 | 165203356 | 165203843 | 162761 |
| 35132 | 165204426 | 165205759 | 162761 |
| 35133 | 165227069 | 165227612 | 162761 |
| 35134 | 165228157 | 165228889 | 162761 |
| 35135 | 165256695 | 165257349 | 162761 |
| 35136 | 165257443 | 165258639 | 162761 |
| 35137 | 165395770 | 165396612 | 143171 |
| 35138 | 165566563 | 165568467 | 236206 |
| 35139 | 165599135 | 165601778 | 143198 |
| 35140 | 165605830 | 165607837 | 143198 |
| 35141 | 165614761 | 165615638 | 143198 |
| 35142 | 165665184 | 165666994 | 143149 |
| 35143 | 165667062 | 165668273 | 215838, 143149 |
| 35144 | 165716642 | 165717389 | 143183 |
| 35145 | 165737345 | 165739387 | 224358, 143183 |
| 35146 | 165741726 | 165743106 | 143183, 224358 |
| 35147 | 165796165 | 165799043 | 143179, 143183 |
| 35148 | 165825542 | 165828896 | 143179 |
| 35149 | 165840538 | 165841216 | 143179 |
| 35150 | 165852343 | 165852703 | 143179 |
| 35151 | 165867843 | 165868514 | 143179, 236364 |
| 35152 | 165879895 | 165881438 | 143179 |
| 35153 | 166055179 | 166055917 | 188859, 203307 |
| 35154 | 166080641 | 166081260 | 188859 |
| 35155 | 166135144 | 166136788 | 188859 |
| 35156 | 166439352 | 166440181 | |
| 35157 | 166457000 | 166459973 | 234142 |
| 35158 | 166783819 | 166784350 | |
| 35159 | 166807449 | 166809731 | 143157 |
| 35160 | 166844517 | 166845989 | 152382 |
| 35161 | 166944210 | 166945506 | 143195 |
| 35162 | 166949791 | 166950621 | |
| 35163 | 166980757 | 166981159 | 143194 |
| 35164 | 167013021 | 167014089 | |
| 35165 | 167188743 | 167192179 | 143190, 272205 |
| 35166 | 167236920 | 167237659 | 143190 |
| 35167 | 167266172 | 167267185 | 143190 |
| 35168 | 167355927 | 167357294 | 143190 |
| 35169 | 167423649 | 167425713 | 198821 |
| 35170 | 167501507 | 167502064 | 143162 |
| 35171 | 167508031 | 167509106 | 143162 |
| 35172 | 167511488 | 167512309 | 143162 |
| 35173 | 167517019 | 167518721 | 143162 |
| 35174 | 167522187 | 167523399 | 143162 |
| 35175 | 167569220 | 167569899 | |
| 35176 | 167682692 | 167685846 | |
| 35177 | 167690067 | 167692519 | 197965 |
| 35178 | 167721422 | 167722732 | 197965 |
| 35179 | 167766133 | 167766824 | |
| 35180 | 167772416 | 167773784 | |
| 35181 | 167789092 | 167789944 | 250762, 232194, 143199 |
| 35182 | 167868062 | 167869062 | 143199 |
| 35183 | 167873808 | 167875141 | 143199 |
| 35184 | 167879240 | 167880044 | 143199 |
| 35185 | 167882950 | 167883908 | 143199 |
| 35186 | 167885832 | 167886974 | 143158 |
| 35187 | 167895619 | 167896382 | 143158 |
| 35188 | 167903053 | 167903372 | 143158 |
| 35189 | 167903661 | 167907150 | 143164, 143158 |
| 35190 | 167963449 | 167964574 | 143164 |
| 35191 | 168147630 | 168149727 | 143155 |
| 35192 | 168194474 | 168196182 | 213064 |
| 35193 | 168199112 | 168201347 | 213064 |
| 35194 | 168204497 | 168205756 | 213064 |
| 35195 | 168337145 | 168337958 | |
| 35196 | 168348174 | 168348893 | |
| 35197 | 168464407 | 168465313 | 237658 |
| 35198 | 168605320 | 168607071 | |
| 35199 | 168662343 | 168662841 | |
| 35200 | 168768988 | 168771067 | |
| 35201 | 168803461 | 168804117 | |
| 35202 | 168889515 | 168890336 | 203601 |
| 35203 | 168892574 | 168893389 | 203601 |
| 35204 | 169074350 | 169084218 | 143153 |
| 35205 | 169084961 | 169086263 | 143153 |
| 35206 | 169114014 | 169114921 | 143156 |
| 35207 | 169116784 | 169118534 | 143156 |
| 35208 | 169203933 | 169205370 | 143156 |
| 35209 | 169208533 | 169210918 | 143156 |
| 35210 | 169336815 | 169338160 | 117475, 143156 |
| 35211 | 169452609 | 169456916 | 213062, 117479 |
| 35212 | 169463753 | 169465876 | |
| 35213 | 169478334 | 169482286 | |
| 35214 | 169497853 | 169499426 | 198734 |
| 35215 | 169515128 | 169516010 | 198734 |
| 35216 | 169539044 | 169540209 | 198734 |
| 35217 | 169540623 | 169542428 | 198734 |
| 35218 | 169552754 | 169556670 | 198734 |
| 35219 | 169557045 | 169558601 | 174175 |
| 35220 | 169693083 | 169693867 | 000460, 007908 |
| 35221 | 169714218 | 169715077 | 000460, 007908 |
| 35222 | 169750140 | 169750952 | 000460 |
| 35223 | 169763020 | 169764635 | 171806, 000460 |
| 35224 | 169861711 | 169864154 | 000457 |
| 35225 | 170036285 | 170037253 | 075945 |
| 35226 | 170042983 | 170044347 | 075945 |
| 35227 | 170500399 | 170502057 | 120370, 231407 |
| 35228 | 170506987 | 170508592 | 120370 |
| 35229 | 171051813 | 171052811 | 226552 |
| 35230 | 171053031 | 171054382 | 226552 |
| 35231 | 171059353 | 171061559 | 007933 |
| 35232 | 171110690 | 171112241 | 117507 |
| 35233 | 171129054 | 171130976 | 117507 |
| 35234 | 171164674 | 171165701 | 094963 |
| 35235 | 171179432 | 171180040 | 094963, 225243 |
| 35236 | 171225835 | 171226809 | 010932 |
| 35237 | 171282580 | 171284416 | 076258 |
| 35238 | 171312685 | 171314988 | 225704, 268062 |
| 35239 | 171320823 | 171321721 | |
| 35240 | 171453968 | 171455696 | 117523 |
| 35241 | 171614021 | 171614583 | 034971 |
| 35242 | 171710421 | 171713197 | 117533 |
| 35243 | 171786197 | 171787258 | |
| 35244 | 171974832 | 171975858 | 197959 |
| 35245 | 172326856 | 172328644 | 197959 |
| 35246 | 172328678 | 172329303 | 197959 |
| 35247 | 172384932 | 172388270 | 197959, 135845 |
| 35248 | 172412235 | 172414113 | 135845, 180999 |
| 35249 | 172417051 | 172420895 | 180999 |
| 35250 | 172421130 | 172422994 | 180999 |
| 35251 | 172434404 | 172438263 | 180999 |
| 35252 | 172438665 | 172440124 | |
| 35253 | 172445069 | 172446136 | |
| 35254 | 172467791 | 172471156 | |
| 35255 | 172500649 | 172503519 | 094975 |
| 35256 | 172591677 | 172592091 | |
| 35257 | 172685366 | 172689197 | |
| 35258 | 172714757 | 172715975 | |
| 35259 | 172792592 | 172793124 | 224228 |
| 35260 | 172886834 | 172887928 | 224228 |
| 35261 | 172901812 | 172904079 | 224228 |
| 35262 | 172920792 | 172921366 | 224228 |
| 35263 | 172944767 | 172946290 | 224228 |
| 35264 | 173021633 | 173022417 | 224228 |
| 35265 | 173091544 | 173093296 | |
| 35266 | 173096324 | 173097909 | |
| 35267 | 173159002 | 173161089 | 117586 |
| 35268 | 173398504 | 173399583 | 203739 |
| 35269 | 173421043 | 173421599 | 203739 |
| 35270 | 173442660 | 173443711 | |
| 35271 | 173444370 | 173448185 | 117592 |
| 35272 | 173472554 | 173473912 | 162753 |
| 35273 | 173476510 | 173477385 | 162753 |
| 35274 | 173492271 | 173493338 | 162753 |
| 35275 | 173496177 | 173496858 | 162753 |
| 35276 | 173683569 | 173685408 | 076321 |
| 35277 | 173722581 | 173723590 | 076321 |
| 35278 | 173793001 | 173794814 | 117593, 120334 |
| 35279 | 173835692 | 173838474 | 185278, 234741 |
| 35280 | 173883389 | 173884190 | 117601 |
| 35281 | 173885562 | 173887360 | 117601 |
| 35282 | 173894231 | 173896411 | |
| 35283 | 173990260 | 173992474 | 224977, 135870 |
| 35284 | 174127894 | 174129781 | 152061, 227373 |
| 35285 | 174236275 | 174236873 | 152061 |
| 35286 | 174695079 | 174696275 | 152061 |
| 35287 | 174781611 | 174782381 | 152061 |
| 35288 | 174922825 | 174923552 | 229531, 152061 |
| 35289 | 174958890 | 174960485 | 152061 |
| 35290 | 174967413 | 174970220 | 116161 |
| 35291 | 174991668 | 174993415 | 120333, 206659 |
| 35292 | 174998184 | 174999581 | |
| 35293 | 175065139 | 175065838 | 120332 |
| 35294 | 175160949 | 175162708 | 235750 |
| 35295 | 175177983 | 175178840 | |
| 35296 | 175604123 | 175604772 | 116147 |
| 35297 | 176175223 | 176177691 | 236021, 143207 |
| 35298 | 176483644 | 176485661 | 116183 |
| 35299 | 176485815 | 176487773 | 116183 |
| 35300 | 176491447 | 176492287 | 116183 |
| 35301 | 176499827 | 176501010 | 116183 |
| 35302 | 176501678 | 176502673 | 116183 |
| 35303 | 176528760 | 176529605 | 116183 |
| 35304 | 176736776 | 176737547 | 116183 |
| 35305 | 177882099 | 177882645 | |
| 35306 | 177930268 | 177930842 | 254154, 120341 |
| 35307 | 177937286 | 177940176 | 254154, 120341 |
| 35308 | 177992337 | 177992875 | 242193, 254154 |
| 35309 | 178003479 | 178004337 | 242193, 254154 |
| 35310 | 178006448 | 178008093 | 242193, 254154 |
| 35311 | 178029860 | 178030750 | |
| 35312 | 178062251 | 178064489 | 075391, 224687 |
| 35313 | 178070500 | 178071725 | 075391 |
| 35314 | 178094185 | 178098019 | 075391 |
| 35315 | 178193178 | 178194474 | 075391 |
| 35316 | 178206870 | 178209929 | 075391 |
| 35317 | 178386482 | 178387637 | 075391 |
| 35318 | 178497911 | 178500348 | 240021 |
| 35319 | 178501099 | 178502193 | 240021 |
| 35320 | 178505073 | 178509052 | 240021 |
| 35321 | 178509517 | 178512851 | 213057, 184909, 240021 |
| 35322 | 178613212 | 178613793 | |
| 35323 | 178621723 | 178623201 | |
| 35324 | 178624806 | 178625805 | |
| 35325 | 178643574 | 178644339 | |
| 35326 | 178652255 | 178655031 | |
| 35327 | 178693523 | 178696956 | 116191, 273062 |
| 35328 | 178708393 | 178708883 | 116191 |
| 35329 | 178836602 | 178838745 | 116191, 116194 |
| 35330 | 178840688 | 178841378 | 116191 |
| 35331 | 178919537 | 178920539 | |
| 35332 | 178971527 | 178972662 | |
| 35333 | 178994657 | 178997163 | 116199 |
| 35334 | 179000458 | 179001597 | 116199 |
| 35335 | 179050342 | 179052384 | 186283 |
| 35336 | 179058868 | 179059574 | 186283 |
| 35337 | 179060502 | 179061880 | 186283 |
| 35338 | 179095317 | 179095962 | 143322 |
| 35339 | 179097567 | 179099259 | 143322 |
| 35340 | 179120650 | 179121699 | 143322 |
| 35341 | 179143856 | 179145304 | 143322 |
| 35342 | 179197946 | 179199468 | 143322 |
| 35343 | 179262502 | 179264047 | 057252 |
| 35344 | 179334290 | 179335852 | 162779 |
| 35345 | 179842467 | 179843292 | 169905 |
| 35346 | 179844576 | 179847523 | 169905 |
| 35347 | 179850178 | 179853106 | 143337, 272906 |
| 35348 | 179922973 | 179924918 | 135837, 260360 |
| 35349 | 179996981 | 179997745 | 135837 |
| 35350 | 180123103 | 180124165 | 116260 |
| 35351 | 180125953 | 180127310 | 116260 |
| 35352 | 180221473 | 180222432 | 121454 |
| 35353 | 180260091 | 180261240 | 135847 |
| 35354 | 180278376 | 180279762 | 135847 |
| 35355 | 180470751 | 180472893 | 135847 |
| 35356 | 180484847 | 180485975 | |
| 35357 | 180490612 | 180492274 | |
| 35358 | 180496339 | 180496950 | |
| 35359 | 180499223 | 180500228 | |
| 35360 | 180567603 | 180568312 | |
| 35361 | 180600953 | 180602339 | 143324 |
| 35362 | 180831253 | 180831813 | 143324 |
| 35363 | 180922578 | 180924588 | 232586, 243155 |
| 35364 | 180990906 | 180992387 | 135823 |
| 35365 | 181013437 | 181014320 | 153029 |
| 35366 | 181057307 | 181060304 | 272198, 162783 |
| 35367 | 181061905 | 181062595 | 272198 |
| 35368 | 181073641 | 181074812 | |
| 35369 | 181080844 | 181082104 | |
| 35370 | 181095534 | 181096709 | |
| 35371 | 181102749 | 181105640 | |
| 35372 | 181109735 | 181112160 | |
| 35373 | 181224485 | 181225182 | |
| 35374 | 182089605 | 182090397 | |
| 35375 | 182257553 | 182261500 | 228918 |
| 35376 | 182262091 | 182262917 | 228918 |
| 35377 | 182358524 | 182361970 | 135821 |
| 35378 | 182366969 | 182367340 | 203730 |
| 35379 | 182379443 | 182380163 | 203729 |
| 35380 | 182428975 | 182429462 | 121446 |
| 35381 | 182544430 | 182545319 | 135828 |
| 35382 | 182557393 | 182558749 | 135828 |
| 35383 | 182573211 | 182573920 | 143333 |
| 35384 | 182583786 | 182584939 | 261504 |
| 35385 | 182758115 | 182759623 | 135838 |
| 35386 | 182808204 | 182809604 | 135829 |
| 35387 | 182931315 | 182932663 | |
| 35388 | 182988222 | 182989467 | |
| 35389 | 182990792 | 182995981 | 135862 |
| 35390 | 182997847 | 183000031 | 135862 |
| 35391 | 183029585 | 183030294 | 135862 |
| 35392 | 183038898 | 183040014 | 135862 |
| 35393 | 183049514 | 183049901 | 135862 |
| 35394 | 183106067 | 183110139 | 224468, 135862 |
| 35395 | 183148854 | 183150681 | |
| 35396 | 183154546 | 183156289 | 058085 |
| 35397 | 183174241 | 183174845 | 058085 |
| 35398 | 183178182 | 183179457 | 058085 |
| 35399 | 183287269 | 183288049 | 157064 |
| 35400 | 183435955 | 183438028 | 232860 |
| 35401 | 183438475 | 183442792 | 116698, 232860 |
| 35402 | 183483404 | 183484792 | 116698 |
| 35403 | 183537642 | 183538736 | 116701, 116698 |
| 35404 | 183569205 | 183569719 | |
| 35405 | 183572212 | 183572804 | |
| 35406 | 183601097 | 183606463 | 143344, 162704 |
| 35407 | 183609056 | 183610089 | 143344 |
| 35408 | 183645556 | 183646280 | 143344 |
| 35409 | 183667911 | 183669791 | 143344 |
| 35410 | 183703542 | 183704190 | 143344 |
| 35411 | 183722275 | 183723006 | 143344 |
| 35412 | 183773897 | 183774503 | 143344 |
| 35413 | 183850585 | 183851257 | 143344 |
| 35414 | 183861407 | 183862012 | 143344 |
| 35415 | 183884573 | 183885590 | 143344 |
| 35416 | 184006251 | 184007067 | 198756 |
| 35417 | 184019600 | 184021744 | 198860 |
| 35418 | 184377210 | 184377706 | 230470, 116667 |
| 35419 | 184460546 | 184462011 | 116667 |
| 35420 | 184462938 | 184463576 | 116667 |
| 35421 | 184559191 | 184560103 | 116667 |
| 35422 | 184591440 | 184592113 | 116667 |
| 35423 | 184594878 | 184597312 | 116667 |
| 35424 | 184598525 | 184599798 | |
| 35425 | 184601221 | 184603881 | |
| 35426 | 184721188 | 184721939 | 116406 |
| 35427 | 184722730 | 184725172 | 116406 |
| 35428 | 184761181 | 184762859 | 135842 |
| 35429 | 184942201 | 184943976 | 135842 |
| 35430 | 185013924 | 185015573 | 121481 |
| 35431 | 185124951 | 185127078 | 116668, 121486 |
| 35432 | 185284426 | 185287328 | 273004, 116679 |
| 35433 | 185446929 | 185447361 | |
| 35434 | 185503879 | 185504370 | |
| 35435 | 185554812 | 185555452 | 228309 |
| 35436 | 185614999 | 185615881 | |
| 35437 | 185662709 | 185663897 | |
| 35438 | 185702882 | 185703552 | |
| 35439 | 185849412 | 185850891 | 143341 |
| 35440 | 185903031 | 185904512 | 143341 |
| 35441 | 186002921 | 186003434 | 143341 |
| 35442 | 186028422 | 186029806 | 143341 |
| 35443 | 186264690 | 186266017 | 116690 |
| 35444 | 186267943 | 186268460 | 116690 |
| 35445 | 186343673 | 186345814 | 157181, 047410 |
| 35446 | 187902325 | 187903440 | |
| 35447 | 192454725 | 192456159 | |
| 35448 | 192599763 | 192600504 | |
| 35449 | 192669293 | 192672248 | |
| 35450 | 192777115 | 192778494 | 116741 |
| 35451 | 193027498 | 193030260 | 116747, 116750 |
| 35452 | 193073250 | 193075583 | 023572 |
| 35453 | 193080153 | 193081816 | |
| 35454 | 193082984 | 193085908 | |
| 35455 | 193090156 | 193092446 | 134371 |
| 35456 | 193323958 | 193325144 | 232077 |
| 35457 | 193377813 | 193378695 | |
| 35458 | 193386688 | 193388464 | |
| 35459 | 193441791 | 193442712 | |
| 35460 | 193527447 | 193528316 | |
| 35461 | 193598840 | 193600145 | |
| 35462 | 193623596 | 193625067 | |
| 35463 | 193995410 | 193996096 | |
| 35464 | 195297224 | 195297681 | |
| 35465 | 196329836 | 196330907 | 162687 |
| 35466 | 196369822 | 196371011 | 162687 |
| 35467 | 196406535 | 196408348 | 162687 |
| 35468 | 196414716 | 196415382 | 162687 |
| 35469 | 196526660 | 196528790 | 162687 |
| 35470 | 196553567 | 196554441 | 162687 |
| 35471 | 196576952 | 196579166 | 162687 |
| 35472 | 196620614 | 196621706 | 000971 |
| 35473 | 196621875 | 196625154 | 000971 |
| 35474 | 196686565 | 196687507 | 000971 |
| 35475 | 196846746 | 196847527 | 134365, 080910 |
| 35476 | 196856283 | 196857561 | 134365, 080910 |
| 35477 | 196944870 | 196948784 | 134389 |
| 35478 | 196960922 | 196962132 | 134389 |
| 35479 | 197035226 | 197037011 | 143278 |
| 35480 | 197099783 | 197100531 | 066279 |
| 35481 | 197115278 | 197115917 | 066279 |
| 35482 | 197142636 | 197144358 | 177888 |
| 35483 | 197169496 | 197171709 | 134376, 177888 |
| 35484 | 197526295 | 197527009 | 213047 |
| 35485 | 197589605 | 197590435 | 213047 |
| 35486 | 197644408 | 197645598 | 213047 |
| 35487 | 197743555 | 197745987 | 213047 |
| 35488 | 197853282 | 197853703 | |
| 35489 | 197870863 | 197872491 | 203724 |
| 35490 | 197923906 | 197925449 | |
| 35491 | 197963396 | 197964817 | |
| 35492 | 197977225 | 197977971 | |
| 35493 | 197999571 | 198000602 | |
| 35494 | 198056865 | 198057964 | |
| 35495 | 198125124 | 198127465 | 151414 |
| 35496 | 198127526 | 198128972 | 151414 |
| 35497 | 198130965 | 198131794 | 151414 |
| 35498 | 198849764 | 198850901 | 229989 |
| 35499 | 199417021 | 199418874 | |
| 35500 | 199615270 | 199616586 | |
| 35501 | 199653653 | 199655043 | |
| 35502 | 199673292 | 199673821 | |
| 35503 | 199720461 | 199722092 | 231984 |
| 35504 | 199727432 | 199729259 | |
| 35505 | 199763244 | 199763772 | |
| 35506 | 199820217 | 199821355 | |
| 35507 | 199834176 | 199835445 | |
| 35508 | 199945514 | 199946537 | |
| 35509 | 199990523 | 199992661 | |
| 35510 | 199996113 | 199999132 | 116833 |
| 35511 | 200007762 | 200012660 | 116833 |
| 35512 | 200045208 | 200045872 | 116833 |
| 35513 | 200059356 | 200059961 | 116833 |
| 35514 | 200087742 | 200088507 | 116833 |
| 35515 | 200099719 | 200101222 | 116833 |
| 35516 | 200119692 | 200120301 | 116833 |
| 35517 | 200148703 | 200150460 | |
| 35518 | 200200354 | 200202140 | |
| 35519 | 200244505 | 200246005 | |
| 35520 | 200265071 | 200266121 | |
| 35521 | 200270458 | 200272573 | |
| 35522 | 200282874 | 200286575 | 273093 |
| 35523 | 200305390 | 200306054 | |
| 35524 | 200320905 | 200321705 | 203721 |
| 35525 | 200334488 | 200336293 | 203721 |
| 35526 | 200336755 | 200339646 | 203721 |
| 35527 | 200340051 | 200344736 | 203721 |
| 35528 | 200344853 | 200347069 | |
| 35529 | 200347232 | 200350779 | |
| 35530 | 200377695 | 200386691 | 162702 |
| 35531 | 200394307 | 200394821 | |
| 35532 | 200411665 | 200412294 | |
| 35533 | 200446639 | 200448049 | 230623 |
| 35534 | 200448706 | 200449546 | |
| 35535 | 200451874 | 200454159 | |
| 35536 | 200608630 | 200610688 | 118197 |
| 35537 | 200637814 | 200640297 | 260088, 118197 |
| 35538 | 200707454 | 200709856 | 118200 |
| 35539 | 200863811 | 200868505 | 163362 |
| 35540 | 200892353 | 200893239 | 233217 |
| 35541 | 200988232 | 200989316 | 116852 |
| 35542 | 200997046 | 200998174 | 229191 |
| 35543 | 201083131 | 201084387 | 232237 |
| 35544 | 201122610 | 201124313 | 116857 |
| 35545 | 201138612 | 201141458 | 116857 |
| 35546 | 201144036 | 201144796 | |
| 35547 | 201153782 | 201154482 | |
| 35548 | 201278280 | 201279001 | 081277 |
| 35549 | 201364834 | 201369518 | 159166 |
| 35550 | 201372747 | 201375112 | 159173 |
| 35551 | 201429070 | 201431464 | |
| 35552 | 201436974 | 201438938 | 174307 |
| 35553 | 201449361 | 201450278 | |
| 35554 | 201450927 | 201452322 | |
| 35555 | 201457447 | 201459366 | 159176 |
| 35556 | 201469738 | 201477501 | 224536, 159176 |
| 35557 | 201481835 | 201482541 | 224536 |
| 35558 | 201507841 | 201508643 | |
| 35559 | 201681703 | 201684261 | 231871, 134369 |
| 35560 | 201797921 | 201799172 | 198700, 231871 |
| 35561 | 201817138 | 201820240 | 198700 |
| 35562 | 201854319 | 201855095 | |
| 35563 | 201857438 | 201858633 | 198892 |
| 35564 | 201864933 | 201865464 | 223774 |
| 35565 | 201923663 | 201925627 | 134375 |
| 35566 | 201951583 | 201952917 | 176393 |
| 35567 | 201975053 | 201975541 | 176393, 234678 |
| 35568 | 201978469 | 201980861 | 249007, 234678, 163435 |
| 35569 | 201985025 | 201985742 | 163435 |
| 35570 | 201986487 | 201988795 | |
| 35571 | 201991629 | 201992693 | |
| 35572 | 202002975 | 202004815 | |
| 35573 | 202006788 | 202007363 | |
| 35574 | 202012396 | 202012787 | |
| 35575 | 202014891 | 202017221 | |
| 35576 | 202025541 | 202028296 | |
| 35577 | 202075567 | 202077851 | |
| 35578 | 202081612 | 202083294 | |
| 35579 | 202087106 | 202088572 | |
| 35580 | 202111813 | 202114501 | 143862 |
| 35581 | 202124262 | 202124909 | 143851 |
| 35582 | 202317402 | 202318788 | 077157 |
| 35583 | 202522736 | 202523199 | 077157 |
| 35584 | 202541580 | 202542534 | 077157 |
| 35585 | 202548311 | 202549791 | 077157 |
| 35586 | 202554669 | 202560409 | 143858, 077157 |
| 35587 | 202757657 | 202758452 | 117139 |
| 35588 | 202775947 | 202777969 | 117139 |
| 35589 | 202779274 | 202780507 | 228288, 260021 |
| 35590 | 202829800 | 202831525 | 234996, 214796 |
| 35591 | 202857166 | 202858532 | 183155 |
| 35592 | 202895228 | 202897018 | 117153 |
| 35593 | 202926624 | 202928141 | 159346 |
| 35594 | 202935647 | 202937392 | 159348 |
| 35595 | 202975208 | 202977466 | 163444 |
| 35596 | 203071189 | 203071732 | 163485 |
| 35597 | 203155693 | 203156681 | 133048 |
| 35598 | 203213868 | 203214365 | 133063 |
| 35599 | 203273764 | 203276272 | 159388, 233791 |
| 35600 | 203504612 | 203508605 | |
| 35601 | 203526508 | 203529057 | |
| 35602 | 203619304 | 203620144 | 058668 |
| 35603 | 203725742 | 203726186 | |
| 35604 | 203738398 | 203739273 | 122188 |
| 35605 | 203764376 | 203766187 | 058673, 257315 |
| 35606 | 203827090 | 203827576 | |
| 35607 | 203829172 | 203832007 | 182004 |
| 35608 | 204042005 | 204045105 | 143842 |
| 35609 | 204049275 | 204050110 | 143842 |
| 35610 | 204053244 | 204053903 | 143842 |
| 35611 | 204061457 | 204063339 | 143842 |
| 35612 | 204095807 | 204097242 | 143842 |
| 35613 | 204097811 | 204100884 | 143845, 261065 |
| 35614 | 204113555 | 204114847 | 143845 |
| 35615 | 204115739 | 204116601 | 143845 |
| 35616 | 204119031 | 204122002 | 143845 |
| 35617 | 204140664 | 204142064 | |
| 35618 | 204182171 | 204183923 | 174567 |
| 35619 | 204194862 | 204195953 | 143850 |
| 35620 | 204223049 | 204223777 | 143850 |
| 35621 | 204235427 | 204236174 | 143850 |
| 35622 | 204255934 | 204257845 | 143850 |
| 35623 | 204263376 | 204263984 | 143850 |
| 35624 | 204264509 | 204265441 | 143850 |
| 35625 | 204345762 | 204348625 | 226330, 143850 |
| 35626 | 204379235 | 204381694 | 158615, 226330 |
| 35627 | 204385380 | 204389940 | 226330 |
| 35628 | 204391461 | 204392839 | 133056, 226330 |
| 35629 | 204394819 | 204396175 | 226330, 133056 |
| 35630 | 204404501 | 204405613 | 226330, 133056 |
| 35631 | 204408870 | 204409683 | 133056 |
| 35632 | 204419581 | 204420770 | 133056 |
| 35633 | 204462478 | 204464433 | 133056 |
| 35634 | 204475240 | 204476732 | |
| 35635 | 204484827 | 204486581 | 198625 |
| 35636 | 204534577 | 204535594 | 198625 |
| 35637 | 204560060 | 204561910 | |
| 35638 | 204572254 | 204572689 | 240710 |
| 35639 | 204592326 | 204592914 | 170382 |
| 35640 | 204653778 | 204655048 | 170382 |
| 35641 | 204677478 | 204678249 | |
| 35642 | 204685716 | 204686407 | |
| 35643 | 204694351 | 204696608 | |
| 35644 | 204775877 | 204776900 | |
| 35645 | 204777082 | 204777754 | |
| 35646 | 204851430 | 204853491 | 163531 |
| 35647 | 204854188 | 204858827 | 163531 |
| 35648 | 204892363 | 204894186 | 163531 |
| 35649 | 204963691 | 204964901 | 163531 |
| 35650 | 204972297 | 204973268 | 163531 |
| 35651 | 205006480 | 205008045 | |
| 35652 | 205089914 | 205092263 | 117222 |
| 35653 | 205113768 | 205114474 | 133059 |
| 35654 | 205119586 | 205120287 | 133059 |
| 35655 | 205160333 | 205161459 | 133059 |
| 35656 | 205179698 | 205181180 | 133059 |
| 35657 | 205241845 | 205244947 | 133069 |
| 35658 | 205252864 | 205254914 | |
| 35659 | 205262251 | 205268627 | |
| 35660 | 205270005 | 205271789 | 163545 |
| 35661 | 205285695 | 205291565 | 163545 |
| 35662 | 205293552 | 205295019 | |
| 35663 | 205461264 | 205461862 | |
| 35664 | 205473346 | 205474602 | 117266 |
| 35665 | 205480811 | 205482185 | 117266 |
| 35666 | 205599365 | 205602150 | 158711 |
| 35667 | 205632701 | 205634780 | 158715 |
| 35668 | 205638590 | 205639179 | 158715 |
| 35669 | 205639230 | 205640548 | 158715 |
| 35670 | 205640909 | 205642676 | 158715 |
| 35671 | 205647681 | 205649906 | 158715 |
| 35672 | 205677896 | 205678545 | |
| 35673 | 205683258 | 205683786 | 268313, 069275 |
| 35674 | 205695498 | 205696229 | 069275 |
| 35675 | 205698086 | 205699326 | 069275 |
| 35676 | 205717566 | 205720235 | 069275 |
| 35677 | 205743617 | 205744760 | 117280 |
| 35678 | 205781506 | 205783146 | 133065 |
| 35679 | 205914081 | 205914618 | |
| 35680 | 206228144 | 206228617 | 198049 |
| 35681 | 206259116 | 206259642 | 196533 |
| 35682 | 206289999 | 206290775 | 240754 |
| 35683 | 206292083 | 206293181 | 240754 |
| 35684 | 206311207 | 206311837 | |
| 35685 | 206643459 | 206644737 | 143466 |
| 35686 | 206654423 | 206654981 | 143466 |
| 35687 | 206669561 | 206670644 | 143466, 162888 |
| 35688 | 206671944 | 206672683 | |
| 35689 | 206674330 | 206675214 | |
| 35690 | 206677005 | 206677900 | 261000 |
| 35691 | 206679036 | 206682698 | 136653 |
| 35692 | 206690947 | 206692072 | 136653 |
| 35693 | 206699335 | 206700308 | 136653 |
| 35694 | 206713547 | 206714244 | 136653 |
| 35695 | 206728552 | 206730768 | 136653 |
| 35696 | 206751598 | 206754447 | 143486, 136653 |
| 35697 | 206784694 | 206786226 | 143486 |
| 35698 | 206806623 | 206809814 | 237605, 143479 |
| 35699 | 206832125 | 206833464 | 143479 |
| 35700 | 206840191 | 206840919 | 143479 |
| 35701 | 206857961 | 206860270 | 162889 |
| 35702 | 206864907 | 206867541 | 162889 |
| 35703 | 206872631 | 206874448 | 162889 |
| 35704 | 206882149 | 206883989 | 162889 |
| 35705 | 206887526 | 206888460 | 162889 |
| 35706 | 206890798 | 206891191 | 162889 |
| 35707 | 206896561 | 206898200 | 162889 |
| 35708 | 207062927 | 207065104 | |
| 35709 | 207115133 | 207115778 | 162896 |
| 35710 | 207122461 | 207123278 | |
| 35711 | 207124572 | 207126203 | |
| 35712 | 207128584 | 207129480 | |
| 35713 | 207142105 | 207142907 | 162897 |
| 35714 | 207143431 | 207144621 | 162897 |
| 35715 | 207198292 | 207199912 | 182795 |
| 35716 | 207223158 | 207227386 | 180667, 123836 |
| 35717 | 207261585 | 207265299 | 123843 |
| 35718 | 207276698 | 207277957 | 123838 |
| 35719 | 207301722 | 207302397 | 123838 |
| 35720 | 207378399 | 207379277 | |
| 35721 | 207412826 | 207415471 | |
| 35722 | 207494055 | 207496133 | 196352 |
| 35723 | 207777059 | 207777681 | 203710, 236911 |
| 35724 | 207880213 | 207881336 | 197721 |
| 35725 | 207923919 | 207926458 | 117335 |
| 35726 | 207979154 | 207980045 | 203709 |
| 35727 | 207985117 | 207986376 | 203709 |
| 35728 | 207996074 | 207998378 | 203709 |
| 35729 | 207998630 | 208001346 | 203709 |
| 35730 | 208041737 | 208043029 | 203709 |
| 35731 | 208063396 | 208064317 | 174059 |
| 35732 | 208136015 | 208137873 | |
| 35733 | 208232387 | 208233426 | 076356 |
| 35734 | 208275071 | 208275962 | 076356 |
| 35735 | 208289270 | 208289733 | 076356 |
| 35736 | 208293052 | 208294396 | 076356 |
| 35737 | 208308812 | 208309234 | 076356 |
| 35738 | 208331938 | 208332793 | 076356 |
| 35739 | 208345260 | 208346558 | 076356 |
| 35740 | 208347306 | 208352461 | 076356 |
| 35741 | 208356143 | 208356793 | 076356 |
| 35742 | 208357560 | 208358908 | 076356 |
| 35743 | 208374350 | 208375190 | 076356 |
| 35744 | 208409280 | 208409926 | 076356 |
| 35745 | 208412100 | 208413023 | 076356 |
| 35746 | 208413026 | 208413873 | 076356 |
| 35747 | 208416000 | 208419145 | 076356 |
| 35748 | 208425456 | 208426612 | |
| 35749 | 208465428 | 208466891 | |
| 35750 | 208505220 | 208506046 | |
| 35751 | 208649616 | 208650579 | |
| 35752 | 209247962 | 209248681 | |
| 35753 | 209269178 | 209270010 | |
| 35754 | 209341287 | 209341983 | |
| 35755 | 209571746 | 209573181 | |
| 35756 | 209576676 | 209577721 | |
| 35757 | 209701347 | 209702018 | 224260 |
| 35758 | 209779300 | 209784635 | 008118 |
| 35759 | 209800392 | 209801903 | 196878 |
| 35760 | 209821485 | 209831883 | 196878 |
| 35761 | 209835658 | 209838112 | 227591 |
| 35762 | 209843955 | 209844648 | 227591 |
| 35763 | 209845539 | 209849696 | 123689, 227591 |
| 35764 | 209877702 | 209879251 | 227591, 117594 |
| 35765 | 209893456 | 209895846 | 227591, 117594 |
| 35766 | 209897321 | 209898298 | 227591, 117594 |
| 35767 | 209914360 | 209917580 | |
| 35768 | 209920506 | 209921718 | |
| 35769 | 209957238 | 209958828 | 162757 |
| 35770 | 209960698 | 209961511 | 232222, 117595 |
| 35771 | 209974368 | 209976115 | 117595 |
| 35772 | 209981412 | 209981924 | |
| 35773 | 210000913 | 210002121 | 117597 |
| 35774 | 210011341 | 210014784 | 117597 |
| 35775 | 210501755 | 210503137 | 054392 |
| 35776 | 210520139 | 210521393 | 054392 |
| 35777 | 210532355 | 210533071 | 054392 |
| 35778 | 210545239 | 210548338 | 200972, 054392 |
| 35779 | 210618536 | 210619223 | 054392 |
| 35780 | 210770362 | 210772069 | 054392 |
| 35781 | 210879317 | 210880241 | 143473 |
| 35782 | 211072975 | 211073590 | 143473 |
| 35783 | 211431499 | 211434619 | 117625 |
| 35784 | 211478218 | 211479328 | 117625 |
| 35785 | 211499604 | 211500844 | 082512 |
| 35786 | 211555674 | 211557207 | 153363 |
| 35787 | 211643238 | 211643816 | |
| 35788 | 211687908 | 211689527 | |
| 35789 | 211689626 | 211690306 | |
| 35790 | 211694610 | 211695313 | |
| 35791 | 211747716 | 211748804 | 170385 |
| 35792 | 211749344 | 211754053 | 170385 |
| 35793 | 211769508 | 211770218 | |
| 35794 | 211781294 | 211782106 | |
| 35795 | 211782435 | 211783279 | |
| 35796 | 211786616 | 211787296 | |
| 35797 | 211805767 | 211807393 | |
| 35798 | 211809301 | 211810620 | 228792 |
| 35799 | 211812787 | 211814082 | 227764, 228792 |
| 35800 | 211816609 | 211817781 | 227764 |
| 35801 | 211820743 | 211824693 | 227764 |
| 35802 | 211848878 | 211849615 | 231057, 117650 |
| 35803 | 212002238 | 212004866 | 229258, 123684 |
| 35804 | 212059103 | 212060124 | |
| 35805 | 212060266 | 212061986 | |
| 35806 | 212076344 | 212078000 | |
| 35807 | 212081957 | 212084700 | |
| 35808 | 212090345 | 212094997 | |
| 35809 | 212101334 | 212103307 | |
| 35810 | 212117497 | 212118669 | 143493 |
| 35811 | 212124681 | 212125901 | 143493 |
| 35812 | 212158625 | 212159495 | 143493 |
| 35813 | 212185222 | 212186093 | 143493 |
| 35814 | 212203668 | 212204769 | 143493 |
| 35815 | 212207667 | 212209459 | 143476, 143493 |
| 35816 | 212332161 | 212332988 | |
| 35817 | 212401744 | 212402712 | 226251 |
| 35818 | 212410739 | 212412476 | 226251 |
| 35819 | 212414960 | 212416874 | |
| 35820 | 212427919 | 212430927 | |
| 35821 | 212433869 | 212435490 | |
| 35822 | 212458052 | 212460566 | 066027 |
| 35823 | 212489622 | 212490323 | 066027, 234915 |
| 35824 | 212587383 | 212588338 | 065600 |
| 35825 | 212605840 | 212607045 | 117691 |
| 35826 | 212658059 | 212658633 | |
| 35827 | 212661607 | 212663048 | |
| 35828 | 212691399 | 212692415 | |
| 35829 | 212730906 | 212733337 | 260805 |
| 35830 | 212768353 | 212771661 | 162772 |
| 35831 | 212779475 | 212783748 | 162772 |
| 35832 | 212838412 | 212839342 | 237980 |
| 35833 | 212872531 | 212873592 | 123685 |
| 35834 | 212894698 | 212895501 | |
| 35835 | 212964250 | 212966284 | 203705, 117697 |
| 35836 | 212989794 | 212990709 | 203705 |
| 35837 | 213006516 | 213007695 | 185523 |
| 35838 | 213030701 | 213032424 | 162769, 198468 |
| 35839 | 213104090 | 213104996 | |
| 35840 | 213188073 | 213189767 | 174606 |
| 35841 | 213223831 | 213225462 | 136643 |
| 35842 | 213296384 | 213297437 | 136643 |
| 35843 | 213374734 | 213375401 | 136643 |
| 35844 | 213449143 | 213450786 | |
| 35845 | 213457932 | 213458374 | |
| 35846 | 213545053 | 213546112 | |
| 35847 | 213743011 | 213744344 | |
| 35848 | 213898445 | 213898826 | |
| 35849 | 213902086 | 213902938 | |
| 35850 | 213932907 | 213933674 | 228255 |
| 35851 | 214031724 | 214032386 | 230461 |
| 35852 | 214107772 | 214108526 | 230461 |
| 35853 | 214129400 | 214130225 | 230461 |
| 35854 | 214131647 | 214132266 | 230461 |
| 35855 | 214149903 | 214151404 | 230461 |
| 35856 | 214155571 | 214164335 | 117707, 230461 |
| 35857 | 214200157 | 214204569 | 272167, 117707 |
| 35858 | 214215851 | 214219842 | |
| 35859 | 214222542 | 214225821 | |
| 35860 | 214241672 | 214242784 | |
| 35861 | 214248666 | 214249503 | |
| 35862 | 214295954 | 214297276 | |
| 35863 | 214299847 | 214300730 | |
| 35864 | 214385974 | 214388658 | |
| 35865 | 214389309 | 214391531 | |
| 35866 | 214453453 | 214455703 | 143499 |
| 35867 | 214458068 | 214458532 | 143499 |
| 35868 | 214485666 | 214486775 | 143499 |
| 35869 | 214488839 | 214491469 | 143499 |
| 35870 | 214611969 | 214613912 | 152104 |
| 35871 | 214724081 | 214726048 | 152104 |
| 35872 | 215038058 | 215038605 | |
| 35873 | 215196644 | 215197310 | 082482 |
| 35874 | 215716418 | 215717157 | |
| 35875 | 215739848 | 215742083 | 136636 |
| 35876 | 215756448 | 215757191 | 136636 |
| 35877 | 215804112 | 215805346 | 042781 |
| 35878 | 215808871 | 215811818 | 042781 |
| 35879 | 215813719 | 215815280 | 042781 |
| 35880 | 215816945 | 215818054 | 042781 |
| 35881 | 215833253 | 215834040 | 042781 |
| 35882 | 215834276 | 215834984 | 042781 |
| 35883 | 216038332 | 216038908 | 042781 |
| 35884 | 216593380 | 216594442 | 042781 |
| 35885 | 216672744 | 216673591 | |
| 35886 | 216773627 | 216775106 | 196482 |
| 35887 | 217803770 | 217805104 | 162814, 092978 |
| 35888 | 218457677 | 218459498 | 067533 |
| 35889 | 218518025 | 218522843 | 092969, 232480 |
| 35890 | 218552776 | 218554808 | 092969 |
| 35891 | 218570701 | 218571898 | 092969 |
| 35892 | 218632807 | 218633620 | |
| 35893 | 218646720 | 218655390 | |
| 35894 | 218660074 | 218664136 | |
| 35895 | 218667272 | 218673672 | |
| 35896 | 218809554 | 218810609 | |
| 35897 | 218819685 | 218820271 | |
| 35898 | 218878064 | 218881935 | |
| 35899 | 218883683 | 218886798 | |
| 35900 | 218908366 | 218909221 | |
| 35901 | 219038846 | 219039543 | |
| 35902 | 219247235 | 219249075 | |
| 35903 | 219260413 | 219266158 | 228063 |
| 35904 | 219346421 | 219351143 | 143353, 228063 |
| 35905 | 219352485 | 219353152 | 143353 |
| 35906 | 219360226 | 219364477 | 143353 |
| 35907 | 219410095 | 219411555 | |
| 35908 | 219483417 | 219484100 | |
| 35909 | 220090347 | 220092325 | 196660 |
| 35910 | 220099066 | 220102954 | 196660 |
| 35911 | 220113592 | 220115862 | 196660 |
| 35912 | 220128016 | 220129014 | 196660 |
| 35913 | 220166821 | 220167455 | 136628 |
| 35914 | 220180383 | 220181207 | 136628 |
| 35915 | 220218834 | 220220455 | 136628 |
| 35916 | 220262474 | 220263747 | 162813 |
| 35917 | 220266643 | 220268218 | 067704 |
| 35918 | 220444683 | 220446503 | 118873 |
| 35919 | 220473769 | 220474238 | |
| 35920 | 220854383 | 220855459 | |
| 35921 | 220863263 | 220864930 | 162817 |
| 35922 | 220871919 | 220872577 | 162817 |
| 35923 | 220879136 | 220879586 | |
| 35924 | 220905400 | 220908897 | |
| 35925 | 220920704 | 220922879 | 117791 |
| 35926 | 220924164 | 220925380 | 117791 |
| 35927 | 220935093 | 220935844 | 117791 |
| 35928 | 220940311 | 220942065 | 117791 |
| 35929 | 220959338 | 220961086 | 186205 |
| 35930 | 220961220 | 220961943 | 186205 |
| 35931 | 220963068 | 220963493 | 186205 |
| 35932 | 220982295 | 220984693 | 186205 |
| 35933 | 220997760 | 220998457 | |
| 35934 | 221000785 | 221001998 | |
| 35935 | 221045388 | 221046191 | 257551 |
| 35936 | 221051553 | 221055158 | 136630, 257551 |
| 35937 | 221079274 | 221080988 | |
| 35938 | 221094091 | 221095370 | |
| 35939 | 221133215 | 221137997 | |
| 35940 | 221139139 | 221141225 | |
| 35941 | 221214096 | 221214817 | |
| 35942 | 221290331 | 221292459 | |
| 35943 | 221390155 | 221390833 | |
| 35944 | 221403568 | 221404500 | |
| 35945 | 221488487 | 221490847 | |
| 35946 | 221508513 | 221510081 | 234754 |
| 35947 | 221521034 | 221521508 | |
| 35948 | 221539852 | 221540514 | |
| 35949 | 221611999 | 221614602 | |
| 35950 | 221661093 | 221661613 | |
| 35951 | 221721698 | 221723324 | 227585 |
| 35952 | 221734965 | 221737818 | |
| 35953 | 221740673 | 221741956 | |
| 35954 | 221776109 | 221776690 | |
| 35955 | 221793043 | 221795621 | |
| 35956 | 221840852 | 221841678 | |
| 35957 | 221849099 | 221850201 | |
| 35958 | 221870917 | 221873431 | |
| 35959 | 221881417 | 221882991 | 143507 |
| 35960 | 221888370 | 221892555 | 143507 |
| 35961 | 221912221 | 221917722 | 143507 |
| 35962 | 221947929 | 221949158 | |
| 35963 | 221996429 | 221999146 | |
| 35964 | 222012249 | 222015164 | 227925 |
| 35965 | 222018340 | 222020686 | |
| 35966 | 222059196 | 222061673 | 238042 |
| 35967 | 222110902 | 222111291 | 238042 |
| 35968 | 222134482 | 222136068 | 238042 |
| 35969 | 222138065 | 222139382 | 238042 |
| 35970 | 222199421 | 222200135 | |
| 35971 | 222212255 | 222212727 | |
| 35972 | 222231208 | 222232085 | 232679 |
| 35973 | 222234602 | 222236505 | 232679 |
| 35974 | 222244555 | 222245314 | |
| 35975 | 222278645 | 222279258 | 236230 |
| 35976 | 222628332 | 222629381 | |
| 35977 | 222638097 | 222640015 | |
| 35978 | 222694219 | 222696780 | 143512 |
| 35979 | 222729329 | 222730254 | |
| 35980 | 222754850 | 222756086 | 143498 |
| 35981 | 222762321 | 222764246 | 225265, 143498 |
| 35982 | 222790280 | 222792335 | 154305 |
| 35983 | 222816508 | 222818464 | 154305 |
| 35984 | 222884478 | 222887493 | 162819, 186063 |
| 35985 | 222950803 | 222951763 | |
| 35986 | 222987416 | 222989408 | 228106, 154309 |
| 35987 | 223101372 | 223102062 | 154309 |
| 35988 | 223173743 | 223174729 | 154309 |
| 35989 | 223180339 | 223181164 | |
| 35990 | 223184459 | 223185058 | |
| 35991 | 223213369 | 223213957 | |
| 35992 | 223218885 | 223220003 | |
| 35993 | 223221196 | 223221884 | |
| 35994 | 223254141 | 223255287 | |
| 35995 | 223350916 | 223352096 | |
| 35996 | 223353525 | 223355443 | 226601 |
| 35997 | 223421509 | 223422899 | 143502 |
| 35998 | 223423166 | 223424061 | 143502 |
| 35999 | 223485427 | 223486230 | 143502 |
| 36000 | 223537029 | 223537984 | 143502 |
| 36001 | 223575061 | 223576734 | |
| 36002 | 223590722 | 223591437 | |
| 36003 | 223808618 | 223811154 | 203697 |
| 36004 | 223822800 | 223823361 | 203697 |
| 36005 | 223826956 | 223830955 | 203697 |
| 36006 | 223840987 | 223844095 | 203697 |
| 36007 | 223894678 | 223896366 | 162909 |
| 36008 | 223899513 | 223902065 | 162909 |
| 36009 | 223908520 | 223909800 | 162909 |
| 36010 | 224002303 | 224004896 | 143514 |
| 36011 | 224031619 | 224035105 | 143514 |
| 36012 | 224072821 | 224076871 | |
| 36013 | 224253091 | 224253526 | |
| 36014 | 224294118 | 224295234 | 252484 |
| 36015 | 224301423 | 224303131 | 143756 |
| 36016 | 224356445 | 224357216 | |
| 36017 | 224370216 | 224372218 | 143753 |
| 36018 | 224400145 | 224400682 | 232628 |
| 36019 | 224517029 | 224519031 | 143748 |
| 36020 | 224544388 | 224545749 | 143771 |
| 36021 | 224618187 | 224619550 | 162923 |
| 36022 | 224620745 | 224623598 | 143786, 162923 |
| 36023 | 224626439 | 224627132 | 143786 |
| 36024 | 224644151 | 224645678 | 143786 |
| 36025 | 224690673 | 224692060 | 143786 |
| 36026 | 224693040 | 224694414 | 143786 |
| 36027 | 224700268 | 224700857 | 143786 |
| 36028 | 224895331 | 224896533 | 143786 |
| 36029 | 224917885 | 224922110 | 229400, 143786 |
| 36030 | 224969380 | 224970137 | |
| 36031 | 225116874 | 225118306 | 185842 |
| 36032 | 225600316 | 225601248 | 203691, 143815 |
| 36033 | 225614709 | 225617132 | 143815 |
| 36034 | 225627973 | 225630272 | |
| 36035 | 225632508 | 225633371 | |
| 36036 | 225641675 | 225643046 | 234476 |
| 36037 | 225695499 | 225697982 | 154380 |
| 36038 | 225839463 | 225842422 | 154380 |
| 36039 | 225842699 | 225843986 | |
| 36040 | 225858756 | 225859850 | |
| 36041 | 225878849 | 225880078 | |
| 36042 | 225880729 | 225881314 | |
| 36043 | 225881758 | 225882515 | |
| 36044 | 225922769 | 225925513 | 226349, 227496 |
| 36045 | 225934018 | 225935351 | 227496 |
| 36046 | 225942506 | 225943546 | |
| 36047 | 225964969 | 225966648 | 143742 |
| 36048 | 225997559 | 225999716 | 143819 |
| 36049 | 226001030 | 226001784 | 143819 |
| 36050 | 226004039 | 226006777 | 143819 |
| 36051 | 226008245 | 226009058 | 143819 |
| 36052 | 226012402 | 226013275 | 143819 |
| 36053 | 226014052 | 226014805 | 143819 |
| 36054 | 226065780 | 226067651 | 196187 |
| 36055 | 226068761 | 226071222 | 196187 |
| 36056 | 226073702 | 226074546 | 255835, 243709 |
| 36057 | 226077348 | 226079388 | 255835, 243709 |
| 36058 | 226084631 | 226085329 | 255835, 243709 |
| 36059 | 226111000 | 226112851 | 143811, 255835 |
| 36060 | 226186008 | 226187655 | 143751 |
| 36061 | 226216507 | 226217815 | |
| 36062 | 226221883 | 226222876 | |
| 36063 | 226233876 | 226234398 | |
| 36064 | 226249078 | 226251609 | 163041, 272562 |
| 36065 | 226270471 | 226271916 | 225518 |
| 36066 | 226297274 | 226298738 | |
| 36067 | 226308976 | 226310937 | |
| 36068 | 226315180 | 226316151 | |
| 36069 | 226322435 | 226323715 | |
| 36070 | 226373215 | 226375212 | 182827 |
| 36071 | 226495992 | 226497421 | 183814 |
| 36072 | 226593626 | 226596472 | 143799 |
| 36073 | 226675481 | 226676538 | |
| 36074 | 226729701 | 226730872 | |
| 36075 | 226890567 | 226891983 | 143772 |
| 36076 | 226898720 | 226899349 | 143772 |
| 36077 | 227051492 | 227055035 | |
| 36078 | 227055110 | 227055814 | |
| 36079 | 227057451 | 227060120 | 143801 |
| 36080 | 227064336 | 227065868 | 143801 |
| 36081 | 227069815 | 227071195 | 143801 |
| 36082 | 227125904 | 227129171 | 163050 |
| 36083 | 227132191 | 227135116 | 163050 |
| 36084 | 227152399 | 227153081 | 163050 |
| 36085 | 227155298 | 227155807 | 163050 |
| 36086 | 227161508 | 227163003 | 163050 |
| 36087 | 227177763 | 227178393 | 143776 |
| 36088 | 227430733 | 227432300 | 143776 |
| 36089 | 227503323 | 227507075 | 143776 |
| 36090 | 227510223 | 227511378 | |
| 36091 | 227531644 | 227532211 | |
| 36092 | 227729304 | 227730930 | |
| 36093 | 227750966 | 227752052 | 181450 |
| 36094 | 227915710 | 227917109 | 143740 |
| 36095 | 227922057 | 227924273 | 081692, 143740 |
| 36096 | 228122345 | 228123720 | 143816 |
| 36097 | 228269124 | 228272228 | 143761 |
| 36098 | 228290236 | 228291268 | 143793 |
| 36099 | 228295855 | 228298159 | 162910 |
| 36100 | 228318614 | 228319189 | |
| 36101 | 228327329 | 228329586 | 143774 |
| 36102 | 228352868 | 228354994 | 181873, 203684 |
| 36103 | 228593197 | 228595233 | 231563, 154370 |
| 36104 | 228611040 | 228611707 | |
| 36105 | 228639906 | 228640883 | |
| 36106 | 228673730 | 228676996 | 168159 |
| 36107 | 228859395 | 228860292 | |
| 36108 | 228870235 | 228874002 | 116574 |
| 36109 | 228918394 | 228919787 | |
| 36110 | 228939153 | 228939651 | |
| 36111 | 228945422 | 228946236 | |
| 36112 | 228956952 | 228957863 | |
| 36113 | 228966728 | 228967455 | |
| 36114 | 228972108 | 228975716 | |
| 36115 | 228977339 | 228978841 | |
| 36116 | 228996377 | 228997814 | |
| 36117 | 229002190 | 229002694 | |
| 36118 | 229038607 | 229039278 | |
| 36119 | 229070448 | 229072350 | |
| 36120 | 229099164 | 229101016 | |
| 36121 | 229162784 | 229163883 | |
| 36122 | 229234466 | 229235345 | |
| 36123 | 229238443 | 229239272 | |
| 36124 | 229244367 | 229245349 | |
| 36125 | 229248521 | 229249418 | |
| 36126 | 229252961 | 229254014 | |
| 36127 | 229260487 | 229260932 | |
| 36128 | 229265631 | 229266502 | |
| 36129 | 229267142 | 229268145 | |
| 36130 | 229283685 | 229284501 | |
| 36131 | 229326729 | 229329116 | |
| 36132 | 229355874 | 229358363 | |
| 36133 | 229358740 | 229359801 | 233920 |
| 36134 | 229360129 | 229361586 | 233920 |
| 36135 | 229365046 | 229365992 | |
| 36136 | 229405550 | 229408246 | 240524, 168118, 177788 |
| 36137 | 229409361 | 229409994 | 168118 |
| 36138 | 229415720 | 229416182 | 168118 |
| 36139 | 229431410 | 229433917 | 168118 |
| 36140 | 229433988 | 229435281 | 168118 |
| 36141 | 229446507 | 229447202 | |
| 36142 | 229477678 | 229479099 | 154429 |
| 36143 | 229643414 | 229645802 | 223635, 069248 |
| 36144 | 229693164 | 229695442 | 135776 |
| 36145 | 229761563 | 229762807 | 135763, 135801 |
| 36146 | 230023008 | 230025537 | |
| 36147 | 230040690 | 230042026 | |
| 36148 | 230146628 | 230148348 | |
| 36149 | 230202043 | 230206711 | 143641 |
| 36150 | 230208289 | 230210218 | 143641 |
| 36151 | 230218475 | 230222445 | 143641 |
| 36152 | 230225721 | 230228084 | 143641 |
| 36153 | 230239254 | 230242976 | 143641 |
| 36154 | 230248331 | 230250793 | 143641 |
| 36155 | 230256663 | 230257153 | 143641 |
| 36156 | 230266961 | 230267566 | 143641 |
| 36157 | 230269506 | 230270058 | 143641 |
| 36158 | 230289700 | 230290133 | 143641 |
| 36159 | 230294178 | 230295868 | 143641 |
| 36160 | 230296590 | 230297837 | 143641 |
| 36161 | 230320999 | 230322403 | 143641 |
| 36162 | 230462338 | 230464039 | 177614 |
| 36163 | 230485998 | 230486994 | 177614 |
| 36164 | 230487203 | 230487790 | 177614 |
| 36165 | 230516575 | 230518853 | 177614 |
| 36166 | 230561674 | 230562683 | |
| 36167 | 230777885 | 230779277 | 135775 |
| 36168 | 230815761 | 230816893 | 135775 |
| 36169 | 230843537 | 230845244 | 135744 |
| 36170 | 230846777 | 230850791 | 135744, 244137 |
| 36171 | 230860961 | 230861771 | 244137 |
| 36172 | 230864163 | 230865200 | 244137 |
| 36173 | 230886510 | 230887837 | 135773, 244137 |
| 36174 | 230960157 | 230962428 | 225656 |
| 36175 | 230984507 | 230986492 | 119280 |
| 36176 | 231002331 | 231005273 | 119280 |
| 36177 | 231113504 | 231115598 | 173409, 143643 |
| 36178 | 231174992 | 231176242 | 182118 |
| 36179 | 231189131 | 231189983 | |
| 36180 | 231297666 | 231298375 | 119283 |
| 36181 | 231330553 | 231331817 | 119283 |
| 36182 | 231346884 | 231347937 | 119283 |
| 36183 | 231355322 | 231356138 | 119283 |
| 36184 | 231368779 | 231370608 | 143633 |
| 36185 | 231371751 | 231372448 | 143633 |
| 36186 | 231375911 | 231377991 | 116906, 143633 |
| 36187 | 231472504 | 231474927 | 010072, 116903 |
| 36188 | 231518865 | 231522940 | 135766 |
| 36189 | 231528103 | 231532499 | 135766 |
| 36190 | 231555705 | 231559994 | 135766 |
| 36191 | 231663437 | 231665307 | 116918, 270106, 233461 |
| 36192 | 232673859 | 232674747 | 116991 |
| 36193 | 232764763 | 232766944 | |
| 36194 | 232814035 | 232814659 | |
| 36195 | 232817930 | 232818382 | |
| 36196 | 232822870 | 232823555 | |
| 36197 | 232839712 | 232841780 | |
| 36198 | 232844944 | 232845715 | |
| 36199 | 232900155 | 232903657 | |
| 36200 | 232940346 | 232942039 | 212916 |
| 36201 | 232980354 | 232981226 | |
| 36202 | 233004463 | 233005260 | |
| 36203 | 233085382 | 233087079 | 135778 |
| 36204 | 233396721 | 233398173 | 135749 |
| 36205 | 233462713 | 233464501 | 143674 |
| 36206 | 233495144 | 233495751 | 143674 |
| 36207 | 233748972 | 233751738 | 135750 |
| 36208 | 233789864 | 233793345 | 135750 |
| 36209 | 233808200 | 233810427 | 135750 |
| 36210 | 233856470 | 233858522 | |
| 3621 1 | 234044679 | 234045781 | 183780 |
| 36212 | 234321317 | 234322606 | 183780 |
| 36213 | 234456273 | 234456708 | 183780 |
| 36214 | 234508280 | 234510298 | 231663, 168275, 273367 |
| 36215 | 234594078 | 234594627 | 059588 |
| 36216 | 234613323 | 234615270 | 059588 |
| 36217 | 234634194 | 234638140 | |
| 36218 | 234638321 | 234639395 | |
| 36219 | 234640698 | 234641954 | |
| 36220 | 234644980 | 234646438 | |
| 36221 | 234652817 | 234654006 | |
| 36222 | 234657963 | 234659932 | |
| 36223 | 234666776 | 234671169 | 231768 |
| 36224 | 234671722 | 234675308 | |
| 36225 | 234676488 | 234682402 | |
| 36226 | 234699533 | 234702021 | 230404 |
| 36227 | 234734176 | 234750505 | 168264, 228830 |
| 36228 | 234752036 | 234757534 | |
| 36229 | 234767275 | 234767991 | 224939 |
| 36230 | 234774036 | 234776210 | |
| 36231 | 234778583 | 234779948 | |
| 36232 | 234792170 | 234794843 | 228044 |
| 36233 | 234819590 | 234820606 | 230628 |
| 36234 | 234851211 | 234861800 | 227630, 224037 |
| 36235 | 234908056 | 234908606 | |
| 36236 | 234971587 | 234972339 | |
| 36237 | 235009650 | 235013253 | |
| 36238 | 235033732 | 235036583 | |
| 36239 | 235057045 | 235057953 | |
| 36240 | 235060677 | 235062609 | |
| 36241 | 235068624 | 235069723 | |
| 36242 | 235089984 | 235101564 | 238005, 237520 |
| 36243 | 235104835 | 235107659 | 238005 |
| 36244 | 235108315 | 235117588 | 258082 |
| 36245 | 235121995 | 235124340 | |
| 36246 | 235132028 | 235135011 | |
| 36247 | 235152849 | 235155319 | |
| 36248 | 235241889 | 235244005 | |
| 36249 | 235246066 | 235247946 | |
| 36250 | 235291088 | 235292664 | 207181, 173726 |
| 36251 | 235323701 | 235325301 | 188739, 054267 |
| 36252 | 235489881 | 235493167 | 152904, 054267 |
| 36253 | 235530348 | 235531793 | 116957 |
| 36254 | 235565167 | 235567139 | 116957 |
| 36255 | 235571900 | 235573212 | 116957 |
| 36256 | 235574941 | 235575493 | 116957 |
| 36257 | 235592434 | 235593043 | 116957 |
| 36258 | 235666303 | 235669133 | 162885 |
| 36259 | 235871335 | 235873292 | 143669 |
| 36260 | 235876786 | 235878180 | 143669 |
| 36261 | 235883768 | 235884538 | 143669 |
| 36262 | 235945824 | 235946957 | 143669 |
| 36263 | 236028683 | 236031553 | 143669 |
| 36264 | 236046102 | 236047303 | 143669 |
| 36265 | 236055622 | 236057807 | |
| 36266 | 236063886 | 236064673 | |
| 36267 | 236071527 | 236075150 | |
| 36268 | 236080034 | 236084688 | |
| 36269 | 236095370 | 236096237 | |
| 36270 | 236105437 | 236106080 | 237845 |
| 36271 | 236108850 | 236110307 | |
| 36272 | 236147695 | 236148237 | 116962 |
| 36273 | 236179105 | 236180498 | 116962 |
| 36274 | 236181392 | 236182762 | 116962 |
| 36275 | 236202715 | 236203414 | 116962 |
| 36276 | 236221135 | 236221957 | 116962 |
| 36277 | 236221970 | 236223008 | 116962 |
| 36278 | 236226671 | 236229268 | 116962 |
| 36279 | 236305630 | 236306424 | 077585 |
| 36280 | 236332049 | 236332643 | 077585 |
| 36281 | 236339676 | 236340741 | 077585 |
| 36282 | 236444464 | 236445733 | 086619 |
| 36283 | 236496482 | 236496933 | |
| 36284 | 236562780 | 236563292 | 186197 |
| 36285 | 236653101 | 236654710 | |
| 36286 | 236665750 | 236667064 | |
| 36287 | 236670515 | 236671043 | |
| 36288 | 236686129 | 236688731 | 223776, 116977 |
| 36289 | 236694915 | 236696086 | 116977 |
| 36290 | 236766961 | 236768527 | 119285 |
| 36291 | 236924706 | 236925216 | 077522 |
| 36292 | 236957929 | 236960261 | 116984 |
| 36293 | 237025017 | 237026579 | 116984 |
| 36294 | 237068281 | 237069142 | |
| 36295 | 237841116 | 237841873 | 198626 |
| 36296 | 237914305 | 237914931 | 198626 |
| 36297 | 237957040 | 237957775 | 198626 |
| 36298 | 238027514 | 238028543 | 237250 |
| 36299 | 238038482 | 238039221 | 237250 |
| 36300 | 238508203 | 238510064 | |
| 36301 | 238533728 | 238534349 | |
| 36302 | 239327903 | 239328629 | |
| 36303 | 239342147 | 239342912 | |
| 36304 | 239391626 | 239392546 | |
| 36305 | 239396797 | 239397472 | |
| 36306 | 239410475 | 239411128 | 259776 |
| 36307 | 239549400 | 239550415 | 133019 |
| 36308 | 239721864 | 239722512 | 133019 |
| 36309 | 239820406 | 239821098 | 133019 |
| 36310 | 239975824 | 239976292 | 133019 |
| 36311 | 239979041 | 239980289 | 133019 |
| 36312 | 240061229 | 240061910 | 234601, 133019 |
| 36313 | 240075787 | 240078717 | 133019 |
| 36314 | 240637297 | 240637764 | 268365, 155816 |
| 36315 | 240662081 | 240663011 | 180875 |
| 36316 | 240685098 | 240686612 | 180875 |
| 36317 | 240724809 | 240726238 | 180875 |
| 36318 | 240739068 | 240740681 | 180875 |
| 36319 | 240759189 | 240761482 | 180875 |
| 36320 | 240774204 | 240777095 | 180875 |
| 36321 | 240813044 | 240813816 | |
| 36322 | 241090994 | 241091653 | 182901 |
| 36323 | 241319659 | 241320952 | 182901 |
| 36324 | 241354839 | 241355837 | 182901 |
| 36325 | 241645257 | 241647285 | |
| 36326 | 241681811 | 241684082 | 091483 |
| 36327 | 241694373 | 241697227 | 117009 |
| 36328 | 241721557 | 241722242 | 117009 |
| 36329 | 241723499 | 241724440 | 117009 |
| 36330 | 241746170 | 241746875 | 117009 |
| 36331 | 241753711 | 241757477 | 054277, 117009 |
| 36332 | 241766914 | 241767962 | 054277 |
| 36333 | 241773289 | 241773907 | 054277 |
| 36334 | 241774155 | 241777154 | 054277 |
| 36335 | 241777431 | 241779896 | 054277 |
| 36336 | 241802515 | 241806326 | 054277 |
| 36337 | 242010850 | 242011761 | 174371 |
| 36338 | 242161628 | 242162284 | 197769 |
| 36339 | 242998827 | 243000192 | |
| 36340 | 243416411 | 243420302 | 054282, 143702 |
| 36341 | 243443591 | 243444394 | 054282 |
| 36342 | 243517660 | 243518364 | 054282 |
| 36343 | 243616886 | 243618548 | 054282 |
| 36344 | 243618781 | 243620143 | 054282 |
| 36345 | 243646314 | 243647804 | 054282 |
| 36346 | 243669019 | 243669849 | 117020 |
| 36347 | 243894506 | 243895245 | 117020, 232184 |
| 36348 | 243902787 | 243903775 | 117020, 232184 |
| 36349 | 243905999 | 243907127 | 117020 |
| 36350 | 244011755 | 244012657 | 117020 |
| 36351 | 244013187 | 244014669 | 117020 |
| 36352 | 244080718 | 244081130 | 226828 |
| 36353 | 244107340 | 244108077 | 226828 |
| 36354 | 244210011 | 244211871 | 226828 |
| 36355 | 244212036 | 244214048 | |
| 36356 | 244227910 | 244229806 | 268146 |
| 36357 | 244230982 | 244231712 | 268146 |
| 36358 | 244252480 | 244253661 | 229960 |
| 36359 | 244255049 | 244256857 | 229960 |
| 36360 | 244257785 | 244259299 | |
| 36361 | 244267015 | 244268630 | 244066 |
| 36362 | 244371038 | 244371748 | |
| 36363 | 244394163 | 244395785 | |
| 36364 | 244435392 | 244438329 | |
| 36365 | 244466103 | 244467239 | |
| 36366 | 244485934 | 244486798 | |
| 36367 | 244613991 | 244616070 | 035687 |
| 36368 | 244624002 | 244625680 | 179397 |
| 36369 | 244815777 | 244817643 | 121644 |
| 36370 | 244822953 | 244823384 | 121644 |
| 36371 | 244893293 | 244894365 | |
| 36372 | 244955417 | 244956081 | |
| 36373 | 244957586 | 244958361 | |
| 36374 | 244964459 | 244964970 | |
| 36375 | 244996740 | 245000396 | 203667 |
| 36376 | 245025296 | 245028953 | 273175, 153187 |
| 36377 | 245132116 | 245135680 | 203666, 272195 |
| 36378 | 245158721 | 245159415 | 203666 |
| 36379 | 245205664 | 245206536 | 203666 |
| 36380 | 245223850 | 245224612 | 203666 |
| 36381 | 245270188 | 245271141 | 203666 |
| 36382 | 245316197 | 245318354 | 162849 |
| 36383 | 245421519 | 245422245 | 162849 |
| 36384 | 246116640 | 246118164 | 185420 |
| 36385 | 246302027 | 246304203 | 185420 |
| 36386 | 246669919 | 246670743 | 185420 |
| 36387 | 246728595 | 246730825 | 162852, 162851 |
| 36388 | 246745687 | 246747446 | 162852 |
| 36389 | 246771906 | 246772609 | 162852 |
| 36390 | 246858943 | 246860227 | |
| 36391 | 246861862 | 246862907 | |
| 36392 | 246870694 | 246871518 | |
| 36393 | 246872523 | 246873781 | |
| 36394 | 246886977 | 246890874 | 143653 |
| 36395 | 246932296 | 246933124 | |
| 36396 | 246990493 | 246991316 | 223519 |
| 36397 | 247093871 | 247095623 | 153207 |
| 36398 | 247240918 | 247242597 | 135747 |
| 36399 | 247266719 | 247268043 | 188295 |
| 36400 | 247274158 | 247275992 | 221953 |
| 36401 | 247276016 | 247277253 | |
| 36402 | 247373315 | 247374383 | 232347, 227671 |
| 36403 | 247493916 | 247496168 | 162714 |
| 36404 | 249114016 | 249123703 | 264500, 175137 |
| 36405 | 249131667 | 249133959 | 171161 |
| 36406 | 249143360 | 249143985 | 171161 |
| 36407 | 249151482 | 249154032 | 227237, 171163 |
| 36408 | 249166861 | 249169025 | |
| 36409 | 249199674 | 249201418 | 185220 |

The locations of ODSCs on Chromosome 2 are shown in Table 44.

TABLE 44
ODSC Locations on Chromosome 2

| SEQ ID NO. | Start | End | Overlapping Gene(s) |
|---|---|---|---|
| 36410 | 40436 | 40982 | 184731 |
| 36411 | 44533 | 48253 | 184731 |
| 36412 | 260531 | 261378 | 035115 |
| 36413 | 263772 | 265744 | 143727, 035115 |
| 36414 | 284937 | 285343 | 189292 |
| 36415 | 676878 | 678178 | 233296, 151353 |
| 36416 | 1553628 | 1554465 | 228613 |
| 36417 | 1821058 | 1821679 | 186487 |
| 36418 | 2000877 | 2001737 | 186487 |
| 36419 | 3306277 | 3307667 | 032389 |
| 36420 | 3328288 | 3329113 | 032389 |
| 36421 | 3380996 | 3382646 | 032389 |
| 36422 | 3382843 | 3385123 | 171853 |
| 36423 | 3469464 | 3470825 | 171853 |
| 36424 | 3520335 | 3524138 | 235078, 182551 |
| 36425 | 3526708 | 3529222 | 235078 |
| 36426 | 3582750 | 3585261 | 242282, 255767 |
| 36427 | 3604728 | 3606996 | 234171, 171865 |
| 36428 | 3622201 | 3624163 | 171863 |
| 36429 | 3632553 | 3633963 | |
| 36430 | 3699169 | 3700128 | |
| 36431 | 6326454 | 6327482 | |
| 36432 | 6897184 | 6897830 | 205837 |
| 36433 | 7005225 | 7006998 | 134321, 134326 |
| 36434 | 7017068 | 7018489 | 134321 |
| 36435 | 7057269 | 7058255 | 151692, 228203 |
| 36436 | 7171781 | 7172322 | 151692 |
| 36437 | 7932177 | 7932916 | |
| 36438 | 8144352 | 8145500 | 235665 |
| 36439 | 8241797 | 8243991 | 236790, 235665 |
| 36440 | 8258587 | 8263004 | 236790, 235665 |
| 36441 | 8265567 | 8267354 | 236790, 235665 |
| 36442 | 8278177 | 8283702 | 229740, 236790, 235665 |
| 36443 | 8286244 | 8287133 | 236790, 235665 |
| 36444 | 8287759 | 8289534 | 236790, 235665 |
| 36445 | 8333612 | 8334742 | 236790, 235665 |
| 36446 | 8378898 | 8379655 | 236790, 235665 |
| 36447 | 8382617 | 8383592 | 236790, 235665 |
| 36448 | 8389158 | 8389889 | 236790, 235665 |
| 36449 | 8445106 | 8446132 | 236790 |
| 36450 | 8446427 | 8447503 | 236790 |
| 36451 | 8459288 | 8459924 | 236790 |
| 36452 | 8504322 | 8505484 | 236790 |
| 36453 | 8543943 | 8544488 | |
| 36454 | 8551550 | 8554520 | |
| 36455 | 8587717 | 8588281 | |
| 36456 | 8597639 | 8598234 | |
| 36457 | 8598767 | 8599377 | |
| 36458 | 8615325 | 8618033 | |
| 36459 | 8633187 | 8634027 | |
| 36460 | 8665552 | 8666749 | |
| 36461 | 8674980 | 8675895 | |
| 36462 | 8693541 | 8694833 | 231435 |
| 36463 | 8737491 | 8738787 | |
| 36464 | 8749218 | 8750429 | 231083 |
| 36465 | 8764206 | 8766416 | |
| 36466 | 8766743 | 8768270 | |
| 36467 | 8773007 | 8775348 | |
| 36468 | 8796221 | 8797994 | |
| 36469 | 8798499 | 8800764 | |
| 36470 | 8802562 | 8803085 | |
| 36471 | 8815614 | 8821874 | 115738, 235092 |
| 36472 | 8822030 | 8824115 | 115738 |
| 36473 | 8824409 | 8826816 | 115738 |
| 36474 | 8961403 | 8961839 | 134313 |
| 36475 | 8976715 | 8977985 | 134313 |
| 36476 | 9282583 | 9283417 | 240980 |
| 36477 | 9285126 | 9285823 | 240980 |
| 36478 | 9346167 | 9348337 | 151693 |
| 36479 | 9374080 | 9376362 | 151693 |
| 36480 | 9449259 | 9452301 | 151693 |
| 36481 | 9474767 | 9477517 | 151693 |
| 36482 | 9562335 | 9564730 | 119203, 119185 |
| 36483 | 9613938 | 9615854 | 134330 |
| 36484 | 9691962 | 9693315 | 151694 |
| 36485 | 9694927 | 9697089 | 271855, 151694 |
| 36486 | 9768533 | 9771632 | 134308 |
| 36487 | 9843554 | 9844390 | 244260 |
| 36488 | 9976899 | 9977607 | |
| 36489 | 9982825 | 9984510 | 115750 |
| 36490 | 10084306 | 10085021 | |
| 36491 | 10085271 | 10086192 | 134317 |
| 36492 | 10090566 | 10092663 | 134317 |
| 36493 | 10115860 | 10116585 | 134317 |
| 36494 | 10124887 | 10125741 | 134317 |
| 36495 | 10149465 | 10151118 | |
| 36496 | 10159091 | 10159812 | |
| 36497 | 10175279 | 10177557 | |
| 36498 | 10182252 | 10186224 | 172059 |
| 36499 | 10260208 | 10261684 | 272524 |
| 36500 | 10400087 | 10400964 | |
| 36501 | 10410682 | 10411498 | |
| 36502 | 10425386 | 10427154 | |
| 36503 | 10437114 | 10439200 | |
| 36504 | 10440070 | 10444995 | 115756 |
| 36505 | 10470234 | 10471280 | 115756 |
| 36506 | 10508496 | 10509851 | 115756 |
| 36507 | 10542265 | 10543602 | 115756 |
| 36508 | 10544800 | 10549721 | 115756 |
| 36509 | 10554061 | 10554873 | 115756 |
| 36510 | 10587332 | 10591030 | 257135, 115758 |
| 36511 | 10592091 | 10592829 | 217258 |
| 36512 | 10714686 | 10718088 | 115761 |
| 36513 | 10730806 | 10732013 | 234818, 115761 |
| 36514 | 10799953 | 10800470 | 115761 |
| 36515 | 10829110 | 10830959 | 243819, 115761 |
| 36516 | 10923431 | 10924018 | 143870, 143882 |
| 36517 | 10951430 | 10953527 | 143870 |
| 36518 | 10970299 | 10970794 | 143870 |
| 36519 | 11076166 | 11077708 | |
| 36520 | 11272623 | 11273356 | 150873, 145063 |
| 36521 | 11294437 | 11296317 | 162976 |
| 36522 | 11380143 | 11380609 | 134318 |
| 36523 | 11450217 | 11450802 | 134318 |
| 36524 | 11482579 | 11486066 | 134318 |
| 36525 | 11488225 | 11488819 | 134318 |
| 36526 | 11492744 | 11493450 | |
| 36527 | 11497318 | 11498100 | 230952 |
| 36528 | 11510645 | 11512438 | |
| 36529 | 11525416 | 11527522 | |
| 36530 | 11527929 | 11528877 | 231403 |
| 36531 | 11529663 | 11530858 | 231403 |
| 36532 | 11536297 | 11537039 | 224177 |
| 36533 | 11551765 | 11552721 | |
| 36534 | 11605229 | 11607035 | 269074, 169016 |
| 36535 | 11622432 | 11624165 | |
| 36536 | 11638263 | 11639854 | |
| 36537 | 11640574 | 11641534 | |
| 36538 | 11645814 | 11647308 | |
| 36539 | 11679480 | 11680330 | 196208 |
| 36540 | 11816249 | 11816671 | |
| 36541 | 11832957 | 11833800 | 134324 |
| 36542 | 11850285 | 11850941 | 134324 |
| 36543 | 11855966 | 11857793 | 134324 |
| 36544 | 11879111 | 11879541 | 134324 |
| 36545 | 11882567 | 11885593 | 230790, 134324 |
| 36546 | 11885721 | 11887779 | 134324 |
| 36547 | 11969305 | 11971148 | |
| 36548 | 12012682 | 12013470 | 224184 |
| 36549 | 12146733 | 12147944 | 224184 |
| 36550 | 12596384 | 12597882 | 224184 |
| 36551 | 12618716 | 12619345 | 224184 |
| 36552 | 12855898 | 12858159 | 071575, 261117 |
| 36553 | 12865503 | 12866197 | 071575 |
| 36554 | 12958700 | 12959678 | |
| 36555 | 13275382 | 13276489 | |
| 36556 | 14774451 | 14776007 | 265629, 162981 |
| 36557 | 15700242 | 15702243 | 151779 |
| 36558 | 15731201 | 15733170 | 079785 |
| 36559 | 15814028 | 15814874 | 224194 |
| 36560 | 15870408 | 15871231 | 231031 |
| 36561 | 15906042 | 15907013 | |
| 36562 | 15916513 | 15917089 | |
| 36563 | 15920249 | 15923844 | |
| 36564 | 16014549 | 16016114 | |
| 36565 | 16079548 | 16081118 | 134323, 233718 |
| 36566 | 16145994 | 16147775 | 226764 |
| 36567 | 16155632 | 16156089 | 226764 |
| 36568 | 16237063 | 16237479 | 236989 |
| 36569 | 16240051 | 16240690 | 236989 |
| 36570 | 16244212 | 16244885 | 236989 |
| 36571 | 16254924 | 16255680 | |
| 36572 | 16606842 | 16607745 | 224400 |
| 36573 | 16794650 | 16795921 | 197872 |
| 36574 | 16918786 | 16919882 | 223536 |
| 36575 | 16921099 | 16922054 | 223536 |
| 36576 | 16938105 | 16939072 | 223536 |
| 36577 | 16944377 | 16945211 | 223536 |
| 36578 | 16956186 | 16957468 | |
| 36579 | 17223989 | 17225052 | |
| 36580 | 17389740 | 17390475 | |
| 36581 | 17536548 | 17537858 | |
| 36582 | 17660868 | 17661552 | |
| 36583 | 17699080 | 17700277 | 214842 |
| 36584 | 17719656 | 17720792 | 163032 |
| 36585 | 17721134 | 17723347 | 163032 |
| 36586 | 17827294 | 17827788 | 163032 |
| 36587 | 17934122 | 17936169 | 178295, 163029 |
| 36588 | 17936172 | 17939972 | 178295, 163029 |
| 36589 | 17944618 | 17945681 | 178295, 163029 |
| 36590 | 17946541 | 17947211 | 178295, 163029 |
| 36591 | 18059458 | 18059846 | 170745 |
| 36592 | 18480580 | 18481421 | 170745 |
| 36593 | 18490578 | 18491551 | 170745 |
| 36594 | 18526851 | 18528147 | 170745 |
| 36595 | 18699479 | 18702024 | |
| 36596 | 18740539 | 18742542 | 240857, 250741, 185013 |
| 36597 | 18777919 | 18779276 | |
| 36598 | 18804062 | 18806416 | |
| 36599 | 18811320 | 18812565 | |
| 36600 | 18933186 | 18934292 | |
| 36601 | 18981536 | 18982312 | |
| 36602 | 19005382 | 19006334 | |
| 36603 | 19009880 | 19014246 | |
| 36604 | 19014304 | 19016566 | |
| 36605 | 19107846 | 19109439 | |
| 36606 | 19151332 | 19153594 | |
| 36607 | 19171751 | 19173899 | 236204 |
| 36608 | 19248467 | 19249080 | 236204 |
| 36609 | 19258630 | 19259250 | 236204 |
| 36610 | 19340222 | 19341351 | 236204 |
| 36611 | 19518770 | 19519375 | 236204 |
| 36612 | 19546929 | 19548697 | 266738 |
| 36613 | 19561840 | 19563685 | |
| 36614 | 19687403 | 19688487 | 237992 |
| 36615 | 19704106 | 19706049 | |
| 36616 | 19737838 | 19739637 | |
| 36617 | 19740133 | 19741536 | |
| 36618 | 19743364 | 19744102 | |
| 36619 | 19810655 | 19812781 | |
| 36620 | 19849427 | 19849978 | |
| 36621 | 19873022 | 19873501 | |
| 36622 | 19985443 | 19987461 | |
| 36623 | 20055219 | 20057266 | |
| 36624 | 20099750 | 20102851 | 271991, 183891 |
| 36625 | 20133074 | 20133764 | 118965 |
| 36626 | 20189399 | 20190298 | 227210, 118965 |
| 36627 | 20227014 | 20227509 | |
| 36628 | 20249864 | 20252962 | 223734, 068697 |
| 36629 | 20328793 | 20329991 | |
| 36630 | 20335383 | 20335960 | |
| 36631 | 20340364 | 20342286 | |
| 36632 | 20356614 | 20357419 | |
| 36633 | 20378302 | 20381254 | |
| 36634 | 20381293 | 20387280 | |
| 36635 | 20394939 | 20397250 | |
| 36636 | 20405412 | 20411095 | 115884 |
| 36637 | 20411514 | 20418688 | 115884 |
| 36638 | 20420308 | 20426473 | 115884 |
| 36639 | 20491579 | 20492116 | 055917 |
| 36640 | 20548552 | 20551919 | 055917 |
| 36641 | 20555726 | 20556397 | |
| 36642 | 20598829 | 20601193 | |
| 36643 | 20620509 | 20622697 | |
| 36644 | 20629109 | 20632344 | |
| 36645 | 20636652 | 20640904 | |
| 36646 | 20642477 | 20654264 | 228950, 143878 |
| 36647 | 20674495 | 20675798 | |
| 36648 | 20724143 | 20726043 | |
| 36649 | 20735168 | 20735715 | |
| 36650 | 20735904 | 20740987 | |
| 36651 | 20776820 | 20785707 | 118960 |
| 36652 | 20791443 | 20800252 | 231948, 118960 |
| 36653 | 20810712 | 20811557 | 118960 |
| 36654 | 20813289 | 20818700 | 118960 |
| 36655 | 20833569 | 20834500 | 118960 |
| 36656 | 20834950 | 20837391 | 118960 |
| 36657 | 20840989 | 20842370 | 118960 |
| 36658 | 20849778 | 20851903 | 118960 |
| 36659 | 20865428 | 20868112 | 143869 |
| 36660 | 20875152 | 20877601 | 260396 |
| 36661 | 20889357 | 20890107 | 118961 |
| 36662 | 20927566 | 20928669 | 118961 |
| 36663 | 20945542 | 20947233 | 118961 |
| 36664 | 20957166 | 20957999 | 118961 |
| 36665 | 20996007 | 20996674 | 118961 |
| 36666 | 21021569 | 21023503 | 118961 |
| 36667 | 21261454 | 21262662 | 084674 |
| 36668 | 21263528 | 21268445 | 084674 |
| 36669 | 21269727 | 21271442 | |
| 36670 | 21274596 | 21275876 | |
| 36671 | 21289476 | 21290566 | |
| 36672 | 21292338 | 21294805 | |
| 36673 | 21315223 | 21316189 | |
| 36674 | 21319052 | 21321242 | |
| 36675 | 21553278 | 21554468 | 231204, 233005 |
| 36676 | 21618975 | 21619584 | 231204, 233005 |
| 36677 | 21645451 | 21646239 | 231204, 233005 |
| 36678 | 21690624 | 21691317 | 231204, 233005 |
| 36679 | 22278416 | 22279979 | 231200 |
| 36680 | 23289164 | 23290186 | 232451 |
| 36681 | 23291931 | 23292672 | 232451 |
| 36682 | 23341504 | 23342305 | 232451 |
| 36683 | 23344690 | 23346585 | 232451 |
| 36684 | 23349994 | 23351285 | 232451 |
| 36685 | 23354319 | 23355561 | 232451 |
| 36686 | 23355788 | 23356609 | 232451 |
| 36687 | 23368266 | 23369254 | 232451 |
| 36688 | 23420586 | 23424767 | 232451 |
| 36689 | 23429190 | 23432884 | |
| 36690 | 23469557 | 23474193 | |
| 36691 | 23490812 | 23492788 | |
| 36692 | 23507912 | 23510557 | |
| 36693 | 23580691 | 23584938 | 223634 |
| 36694 | 23602142 | 23605353 | 235497 |
| 36695 | 23607287 | 23610164 | 119771 |
| 36696 | 23624801 | 23625551 | 119771 |
| 36697 | 23703246 | 23704415 | 119771 |
| 36698 | 23968473 | 23969108 | |
| 36699 | 24007746 | 24008386 | 119778 |
| 36700 | 24011706 | 24012747 | 119778 |
| 36701 | 24024300 | 24024599 | 119778 |
| 36702 | 24148549 | 24151085 | 173960, 119778 |
| 36703 | 24162785 | 24164228 | 173960 |
| 36704 | 24232356 | 24233857 | 205639 |
| 36705 | 24238142 | 24240293 | 205639 |
| 36706 | 24268836 | 24269295 | 205639, 163026 |
| 36707 | 24269595 | 24270415 | 205639, 163026 |
| 36708 | 24272244 | 24273467 | 119782, 163026, 205639 |
| 36709 | 24298349 | 24299713 | 219626, 115128 |
| 36710 | 24306083 | 24308475 | 219626, 115129 |
| 36711 | 24345639 | 24347197 | 176732, 219626 |
| 36712 | 24490890 | 24493266 | 198399 |
| 36713 | 24526818 | 24527404 | 198399 |
| 36714 | 24540291 | 24541009 | 198399 |
| 36715 | 24562554 | 24563203 | 198399 |
| 36716 | 24582080 | 24584348 | 198399 |
| 36717 | 24713003 | 24715488 | 084676 |
| 36718 | 24944141 | 24944959 | 084676 |
| 36719 | 25015241 | 25016735 | 138092, 184924 |
| 36720 | 25193686 | 25196254 | 224165, 115137 |
| 36721 | 25473920 | 25476076 | 119772 |
| 36722 | 25562599 | 25565728 | 119772 |
| 36723 | 25584759 | 25585586 | |
| 36724 | 25599521 | 25600082 | 138101 |
| 36725 | 25841277 | 25842036 | 138101 |
| 36726 | 25895000 | 25897557 | 138101 |
| 36727 | 26009756 | 26010472 | 143970 |
| 36728 | 26051965 | 26053356 | 143970 |
| 36729 | 26100162 | 26102404 | 143970 |
| 36730 | 26220071 | 26221531 | |
| 36731 | 26222039 | 26223234 | |
| 36732 | 26223278 | 26223892 | |
| 36733 | 26231298 | 26232295 | |
| 36734 | 26234209 | 26236001 | |
| 36735 | 26240097 | 26241809 | |
| 36736 | 26255756 | 26258896 | 084733 |
| 36737 | 26266985 | 26267684 | 084733 |
| 36738 | 26303116 | 26303713 | 084733 |
| 36739 | 26401577 | 26402935 | 157833 |
| 36740 | 26463515 | 26464291 | 084754 |
| 36741 | 26466555 | 26468912 | 084754, 138029 |
| 36742 | 26496512 | 26497582 | 138029 |
| 36743 | 26567184 | 26569938 | 173567, 138018 |
| 36744 | 26586171 | 26586808 | 138018 |
| 36745 | 26634337 | 26635841 | 157856 |
| 36746 | 26678469 | 26680331 | 115155, 157856 |
| 36747 | 26684785 | 26686732 | 115155 |
| 36748 | 26714400 | 26714946 | 115155 |
| 36749 | 26943107 | 26945369 | 171303 |
| 36750 | 26946868 | 26947595 | 171303 |
| 36751 | 26980394 | 26982338 | |
| 36752 | 26986123 | 26989062 | 115163, 213699 |
| 36753 | 26990702 | 26991262 | 115163, 213699 |
| 36754 | 26993392 | 26996472 | 115163, 213699 |
| 36755 | 27193133 | 27195824 | 084764, 230286 |
| 36756 | 27199043 | 27199686 | 084764, 230286 |
| 36757 | 27214113 | 27215212 | 084764, 230286, 205500 |
| 36758 | 27231295 | 27234851 | 230286, 084764, 205500 |
| 36759 | 27234944 | 27240259 | 205500, 084764 |
| 36760 | 27240848 | 27241531 | 084764 |
| 36761 | 27243116 | 27243783 | 084764 |
| 36762 | 27255038 | 27256698 | 119777 |
| 36763 | 27272354 | 27275301 | 231636, 084693 |
| 36764 | 27293294 | 27295690 | 228474, 084693 |
| 36765 | 27303897 | 27304708 | 138080 |
| 36766 | 27308338 | 27309900 | 138030, 138080 |
| 36767 | 27315539 | 27317450 | 138030 |
| 36768 | 27318475 | 27318994 | 138030 |
| 36769 | 27348869 | 27349806 | 143994 |
| 36770 | 27384473 | 27386092 | |
| 36771 | 27403207 | 27404014 | |
| 36772 | 27408199 | 27409100 | |
| 36773 | 27417625 | 27418281 | |
| 36774 | 27433888 | 27436300 | 138085, 138074 |
| 36775 | 27440049 | 27441156 | 084774 |
| 36776 | 27515623 | 27516966 | 138100 |
| 36777 | 27543749 | 27547029 | 115204 |
| 36778 | 27578671 | 27580694 | 234072, 115207 |
| 36779 | 27592161 | 27594789 | 115234, 115211 |
| 36780 | 27602341 | 27604650 | 115241, 163795 |
| 36781 | 27631413 | 27633310 | 115241 |
| 36782 | 27650559 | 27652681 | 115216 |
| 36783 | 27712298 | 27712924 | 138002 |
| 36784 | 27716034 | 27720834 | 084734, 115226 |
| 36785 | 27723900 | 27724388 | 084734 |
| 36786 | 27805280 | 27807090 | 259080, 243943, 221843 |
| 36787 | 27850746 | 27851367 | 198522, 176714, 259080, |
| | | | 243943 |
| 36788 | 27851687 | 27852935 | 176714, 198522, 259080, |
| | | | 243943 |
| 36789 | 27885464 | 27887584 | 163798, 119760 |
| 36790 | 27903426 | 27907616 | 163798 |
| 36791 | 27927659 | 27930610 | 205334 |
| 36792 | 27932819 | 27933690 | 205334 |
| 36793 | 27933694 | 27934472 | 205334 |
| 36794 | 27942243 | 27945525 | |
| 36795 | 27946115 | 27948893 | |
| 36796 | 27957573 | 27962441 | |
| 36797 | 27974948 | 27978352 | |
| 36798 | 27983734 | 27984799 | |
| 36799 | 27985782 | 27986832 | |
| 36800 | 27993604 | 27995679 | 243147 |
| 36801 | 28021062 | 28023228 | 171174, 243147 |
| 36802 | 28076843 | 28079672 | 171174, 243147 |
| 36803 | 28112304 | 28115025 | 158019, 171174, 243147 |
| 36804 | 28139151 | 28139582 | 158019, 243147 |
| 36805 | 28154336 | 28156328 | 158019, 243147 |
| 36806 | 28176671 | 28179141 | 158019, 243147 |
| 36807 | 28200351 | 28201152 | 158019, 243147 |
| 36808 | 28215783 | 28216455 | 158019 |
| 36809 | 28357022 | 28357751 | 158019 |
| 36810 | 28455402 | 28457411 | 158019 |
| 36811 | 28491844 | 28492610 | 158019 |
| 36812 | 28497649 | 28500043 | 158019 |
| 36813 | 28556478 | 28558056 | 158019 |
| 36814 | 28561212 | 28561664 | 158019 |
| 36815 | 28564691 | 28568768 | |
| 36816 | 28578200 | 28579849 | |
| 36817 | 28612568 | 28620126 | 075426, 270640, 229951 |
| 36818 | 28631082 | 28631842 | 075426 |
| 36819 | 28657410 | 28658336 | |
| 36820 | 28658424 | 28659278 | |
| 36821 | 28664860 | 28665867 | |
| 36822 | 28668543 | 28669769 | |
| 36823 | 28789470 | 28790580 | 163803 |
| 36824 | 28805129 | 28809222 | 163803 |
| 36825 | 28843853 | 28845250 | 163803 |
| 36826 | 28856129 | 28858903 | 230730, 163803 |
| 36827 | 28862518 | 28864162 | 230730, 163803 |
| 36828 | 28865900 | 28867172 | 163803, 230730 |
| 36829 | 28884439 | 28885730 | 230730 |
| 36830 | 28890924 | 28891510 | |
| 36831 | 28905875 | 28906633 | |
| 36832 | 28931786 | 28932433 | 226833 |
| 36833 | 28939048 | 28940648 | 226833 |
| 36834 | 28958761 | 28959335 | 226833 |
| 36835 | 28973044 | 28976655 | 213639 |
| 36836 | 29092139 | 29094023 | 171103 |
| 36837 | 29117122 | 29118919 | 163811 |
| 36838 | 29174664 | 29175817 | |
| 36839 | 29345742 | 29347386 | 115295 |
| 36840 | 29352344 | 29353699 | 115295 |
| 36841 | 29741914 | 29742751 | 171094 |
| 36842 | 29946034 | 29947065 | 171094 |
| 36843 | 29947932 | 29949436 | 171094 |
| 36844 | 30369259 | 30371032 | 119801 |
| 36845 | 30378585 | 30379571 | 119801 |
| 36846 | 30456987 | 30457720 | 213626 |
| 36847 | 30496958 | 30498552 | 213626 |
| 36848 | 30669820 | 30671013 | 172954 |
| 36849 | 30672525 | 30673455 | 172954 |
| 36850 | 30944493 | 30945371 | |
| 36851 | 31305966 | 31306866 | 158089 |
| 36852 | 31546453 | 31548118 | |
| 36853 | 31601141 | 31601885 | 158125 |
| 36854 | 31628853 | 31630117 | 158125 |
| 36855 | 31635831 | 31636505 | 158125 |
| 36856 | 31760776 | 31761191 | 228563, 049319 |
| 36857 | 31772207 | 31773068 | 228563, 049319 |
| 36858 | 31781132 | 31787598 | 228563, 049319 |
| 36859 | 31803287 | 31804214 | 049319 |
| 36860 | 31891858 | 31892372 | |
| 36861 | 31928316 | 31931082 | |
| 36862 | 31934869 | 31935673 | |
| 36863 | 31953347 | 31955518 | |
| 36864 | 32036721 | 32037838 | |
| 36865 | 32217024 | 32217561 | 162959, 162961 |
| 36866 | 32233973 | 32235290 | 162959, 162961 |
| 36867 | 32235522 | 32236952 | 162959, 162961 |
| 36868 | 32264381 | 32265566 | 162961 |
| 36869 | 32288085 | 32289821 | 021574 |
| 36870 | 32360471 | 32361242 | 021574 |
| 36871 | 32389901 | 32391954 | 152683, 272716 |
| 36872 | 32502102 | 32503962 | 119820 |
| 36873 | 32554138 | 32554760 | |
| 36874 | 32581106 | 32583337 | 115760 |
| 36875 | 32652824 | 32654028 | 115760 |
| 36876 | 32678674 | 32679171 | 115760 |
| 36877 | 32852747 | 32854029 | 018699 |
| 36878 | 33030199 | 33030802 | 018699 |
| 36879 | 33089608 | 33091785 | 230876 |
| 36880 | 33141117 | 33141656 | 230876 |
| 36881 | 33171153 | 33173369 | 049323 |
| 36882 | 33225882 | 33226532 | 049323 |
| 36883 | 33226797 | 33227579 | 049323 |
| 36884 | 33229984 | 33231538 | 049323 |
| 36885 | 33238814 | 33241735 | 049323 |
| 36886 | 33266750 | 33269225 | 049323 |
| 36887 | 33275892 | 33278152 | 049323 |
| 36888 | 33293068 | 33296901 | 049323 |
| 36889 | 33319227 | 33319956 | 049323 |
| 36890 | 33358093 | 33360443 | 049323 |
| 36891 | 33422511 | 33423096 | 049323 |
| 36892 | 33435078 | 33438096 | 049323 |
| 36893 | 33441512 | 33442711 | 049323 |
| 36894 | 33444081 | 33446082 | 049323 |
| 36895 | 33464965 | 33466171 | 049323 |
| 36896 | 33516221 | 33517108 | 049323 |
| 36897 | 33650126 | 33652123 | |
| 36898 | 33682759 | 33683971 | 152689 |
| 36899 | 33700193 | 33703661 | 152689 |
| 36900 | 33709287 | 33709761 | 152689 |
| 36901 | 33777779 | 33778556 | 152689 |
| 36902 | 33823389 | 33824899 | 119812 |
| 36903 | 34134969 | 34135500 | 203386 |
| 36904 | 34467138 | 34468081 | 203386 |
| 36905 | 34930208 | 34931774 | 228262 |
| 36906 | 35076569 | 35077437 | 226994 |
| 36907 | 36713196 | 36714511 | 150938 |
| 36908 | 36729070 | 36729516 | 150938 |
| 36909 | 36788038 | 36791299 | 171055 |
| 36910 | 36824585 | 36825552 | 171055 |
| 36911 | 36877150 | 36877845 | |
| 36912 | 37048189 | 37048746 | |
| 36913 | 37192901 | 37195151 | 115808 |
| 36914 | 37310787 | 37311888 | 152133, 008869 |
| 36915 | 37330621 | 37331486 | 055332 |
| 36916 | 37383277 | 37385031 | 055332 |
| 36917 | 37398608 | 37399108 | 138068 |
| 36918 | 37423190 | 37424744 | 218739, 138068 |
| 36919 | 37457629 | 37459626 | 003509, 115816 |
| 36920 | 37551059 | 37553257 | 232028, 115825 |
| 36921 | 37616867 | 37618631 | |
| 36922 | 37699163 | 37700509 | |
| 36923 | 37750098 | 37752057 | |
| 36924 | 37897950 | 37900177 | 163171 |
| 36925 | 38013541 | 38015055 | |
| 36926 | 38145737 | 38146417 | |
| 36927 | 38151336 | 38153651 | 115841 |
| 36928 | 38285031 | 38285561 | 235848, 115841 |
| 36929 | 38303060 | 38305158 | 138061, 232973 |
| 36930 | 38325341 | 38326137 | 138061, 232973 |
| 36931 | 38336079 | 38338333 | 138061, 232973 |
| 36932 | 38373040 | 38374963 | 199603, 232973 |
| 36933 | 38375252 | 38376630 | 232973 |
| 36934 | 38401606 | 38402432 | 232973 |
| 36935 | 38443031 | 38443720 | 227292 |
| 36936 | 38602619 | 38605340 | 119787 |
| 36937 | 38633316 | 38635070 | 231367 |
| 36938 | 38635520 | 38636051 | 231367 |
| 36939 | 38659811 | 38661882 | 235009, 231367 |
| 36940 | 38723900 | 38725173 | 231367 |
| 36941 | 38762760 | 38764166 | 271443 |
| 36942 | 38828805 | 38831055 | 143889, 235586 |
| 36943 | 38892793 | 38894701 | 143891 |
| 36944 | 38912265 | 38913317 | 143891 |
| 36945 | 38917371 | 38918424 | 143891 |
| 36946 | 38958730 | 38959328 | 143891 |
| 36947 | 38961985 | 38963171 | 143891 |
| 36948 | 38977204 | 38979016 | 152147, 115875 |
| 36949 | 38998723 | 38999896 | 152147 |
| 36950 | 39004634 | 39006133 | 152147 |
| 36951 | 39011626 | 39012306 | 152147 |
| 36952 | 39020423 | 39022196 | |
| 36953 | 39102266 | 39103663 | 188010, 163214 |
| 36954 | 39229009 | 39230047 | 115904 |
| 36955 | 39346696 | 39349317 | 115904 |
| 36956 | 39350731 | 39352629 | 115904 |
| 36957 | 39368756 | 39369388 | |
| 36958 | 39663068 | 39666015 | 231312, 011566 |
| 36959 | 39694911 | 39695391 | 231312 |
| 36960 | 39696807 | 39697420 | 231312 |
| 36961 | 39718614 | 39720405 | 231312 |
| 36962 | 39730895 | 39734661 | 231312 |
| 36963 | 39738825 | 39741256 | 231312 |
| 36964 | 39749173 | 39749685 | 231312 |
| 36965 | 39777536 | 39778424 | 231312 |
| 36966 | 39795492 | 39796191 | 231312 |
| 36967 | 39798221 | 39798814 | 231312 |
| 36968 | 39835646 | 39836695 | 231312 |
| 36969 | 39869290 | 39869904 | 231312 |
| 36970 | 40005287 | 40006850 | 138050 |
| 36971 | 42067778 | 42068830 | |
| 36972 | 42073851 | 42076334 | |
| 36973 | 42086301 | 42088384 | |
| 36974 | 42121720 | 42123349 | |
| 36975 | 42134846 | 42135380 | |
| 36976 | 42192756 | 42194112 | |
| 36977 | 42274284 | 42276450 | 162878 |
| 36978 | 42319024 | 42320481 | |
| 36979 | 42328153 | 42330678 | |
| 36980 | 42343595 | 42344614 | |
| 36981 | 42345046 | 42345878 | |
| 36982 | 42359403 | 42361107 | |
| 36983 | 42368683 | 42370192 | |
| 36984 | 42374486 | 42375527 | 224875 |
| 36985 | 42396091 | 42397875 | 143924, 224875 |
| 36986 | 42418283 | 42419379 | 143924 |
| 36987 | 42422536 | 42423257 | 143924 |
| 36988 | 42568700 | 42570983 | 115944 |
| 36989 | 42586646 | 42589558 | 115944 |
| 36990 | 42795250 | 42796944 | 057935 |
| 36991 | 43014374 | 43015686 | 162882 |
| 36992 | 43018655 | 43022024 | 162882 |
| 36993 | 43022260 | 43024064 | |
| 36994 | 43036308 | 43038690 | |
| 36995 | 43081108 | 43081893 | |
| 36996 | 43151211 | 43156096 | |
| 36997 | 43184227 | 43185679 | |
| 36998 | 43201972 | 43202837 | |
| 36999 | 43206229 | 43207417 | |
| 37000 | 43232290 | 43233406 | 224739 |
| 37001 | 43279297 | 43279934 | |
| 37002 | 43382487 | 43383670 | |
| 37003 | 43384516 | 43387501 | |
| 37004 | 43405078 | 43407062 | 115970 |
| 37005 | 43420504 | 43422688 | 115970 |
| 37006 | 43445502 | 43457102 | 152518, 234936, 115970 |
| 37007 | 43503159 | 43504618 | 115970 |
| 37008 | 43519254 | 43522496 | 115970 |
| 37009 | 43532165 | 43532635 | 115970 |
| 37010 | 43538371 | 43538956 | 115970 |
| 37011 | 43569277 | 43570053 | 115970 |
| 37012 | 43653379 | 43654836 | 115970 |
| 37013 | 43682357 | 43687032 | 115970 |
| 37014 | 43720830 | 43721779 | 115970 |
| 37015 | 43742473 | 43743684 | 115970 |
| 37016 | 43746259 | 43747722 | 115970 |
| 37017 | 43765501 | 43769285 | 115970 |
| 37018 | 43822187 | 43824209 | 115970 |
| 37019 | 43861651 | 43862324 | |
| 37020 | 44001015 | 44001914 | 138036 |
| 37021 | 44033180 | 44034083 | 138036 |
| 37022 | 44035215 | 44036044 | 138036 |
| 37023 | 44045761 | 44046724 | 138075 |
| 37024 | 44051586 | 44052840 | 138075 |
| 37025 | 44061627 | 44062179 | 138075 |
| 37026 | 44064916 | 44067928 | 143921, 138075 |
| 37027 | 44071213 | 44072170 | 143921 |
| 37028 | 44074104 | 44074763 | 143921 |
| 37029 | 44222187 | 44223967 | 138095 |
| 37030 | 44283090 | 44285758 | |
| 37031 | 44287783 | 44289784 | |
| 37032 | 44302304 | 44303639 | |
| 37033 | 44313749 | 44315418 | |
| 37034 | 44394431 | 44398870 | 138032, 273106 |
| 37035 | 44471052 | 44473072 | 138032 |
| 37036 | 44505551 | 44506035 | 138079 |
| 37037 | 44513915 | 44515386 | 138079 |
| 37038 | 44517346 | 44519977 | 138079 |
| 37039 | 44587662 | 44590117 | 143919, 138078 |
| 37040 | 44750706 | 44752986 | 143919 |
| 37041 | 44770843 | 44772078 | 143919 |
| 37042 | 44843623 | 44844452 | 143919 |
| 37043 | 44865643 | 44866226 | 143919 |
| 37044 | 44986713 | 44987406 | 143919 |
| 37045 | 45013341 | 45013878 | |
| 37046 | 45260254 | 45260912 | |
| 37047 | 45346183 | 45346720 | |
| 37048 | 45465140 | 45465826 | 205054 |
| 37049 | 45483336 | 45484936 | 205054 |
| 37050 | 45500390 | 45505178 | 205054 |
| 37051 | 45557810 | 45558922 | |
| 37052 | 45595888 | 45596458 | |
| 37053 | 45686035 | 45686793 | 068784 |
| 37054 | 45696311 | 45697414 | 068784 |
| 37055 | 45837618 | 45839448 | 068784 |
| 37056 | 45870019 | 45871773 | |
| 37057 | 45876269 | 45880024 | 171132 |
| 37058 | 46005582 | 46006311 | 171132 |
| 37059 | 46042541 | 46043965 | 171132 |
| 37060 | 46048179 | 46051460 | 171132 |
| 37061 | 46099037 | 46099895 | 171132 |
| 37062 | 46139122 | 46139632 | 171132 |
| 37063 | 46162618 | 46164047 | 171132 |
| 37064 | 46164773 | 46166908 | 171132 |
| 37065 | 46186510 | 46192049 | 171132 |
| 37066 | 46196141 | 46196904 | 171132 |
| 37067 | 46218210 | 46221915 | 171132 |
| 37068 | 46226895 | 46237092 | 233829, 171132 |
| 37069 | 46258916 | 46261680 | 171132 |
| 37070 | 46264536 | 46265991 | 171132 |
| 37071 | 46267044 | 46270018 | 171132 |
| 37072 | 46344374 | 46345370 | 171132 |
| 37073 | 46369707 | 46370881 | 171132 |
| 37074 | 46451868 | 46453731 | |
| 37075 | 46456098 | 46457965 | |
| 37076 | 46510636 | 46511295 | |
| 37077 | 46523409 | 46528468 | 116016 |
| 37078 | 46532504 | 46535533 | 116016 |
| 37079 | 46540973 | 46543625 | 116016 |
| 37080 | 46551414 | 46555631 | 116016 |
| 37081 | 46556037 | 46564151 | 116016 |
| 37082 | 46567022 | 46567667 | 116016 |
| 37083 | 46635354 | 46637460 | |
| 37084 | 46669104 | 46669896 | 187600 |
| 37085 | 46715052 | 46715793 | |
| 37086 | 46726501 | 46727799 | 253515, 250565 |
| 37087 | 46767586 | 46770019 | 119729, 250565 |
| 37088 | 46770185 | 46771663 | 119729 |
| 37089 | 46843124 | 46845037 | 119878, 151665 |
| 37090 | 46875194 | 46875667 | |
| 37091 | 46924510 | 46927517 | 171150 |
| 37092 | 46943818 | 46944985 | 171150 |
| 37093 | 46997009 | 46998212 | |
| 37094 | 47073951 | 47075272 | 239332 |
| 37095 | 47093602 | 47094421 | |
| 37096 | 47103947 | 47104663 | |
| 37097 | 47115940 | 47116662 | |
| 37098 | 47136636 | 47138020 | 180398 |
| 37099 | 47140524 | 47143906 | 068724, 180398 |
| 37100 | 47167318 | 47169835 | 180398, 068724 |
| 37101 | 47181679 | 47184837 | 272814, 068724 |
| 37102 | 47191979 | 47193092 | 068724 |
| 37103 | 47194009 | 47196353 | 068724 |
| 37104 | 47196799 | 47199091 | 068724 |
| 37105 | 47212568 | 47213143 | 068724 |
| 37106 | 47214001 | 47215781 | 068724 |
| 37107 | 47256992 | 47257675 | 068724 |
| 37108 | 47260032 | 47263530 | 233845, 068724 |
| 37109 | 47267516 | 47268709 | 233845, 068724 |
| 37110 | 47269454 | 47272007 | 068724 |
| 37111 | 47292445 | 47293731 | 273269, 239605, 068724 |
| 37112 | 47296222 | 47297606 | 239605, 068724, 273269, |
| | | | 225187 |
| 37113 | 47302466 | 47307789 | 068724, 239605, 273269 |
| 37114 | 47336687 | 47337271 | 239605, 273269 |
| 37115 | 47370593 | 47373867 | 239605, 273269 |
| 37116 | 47399137 | 47404937 | 273269, 143933 |
| 37117 | 47416464 | 47418040 | |
| 37118 | 47418409 | 47422919 | 234690 |
| 37119 | 47435536 | 47436523 | 234690 |
| 37120 | 47438239 | 47439385 | 234690 |
| 37121 | 47442767 | 47443672 | 234690 |
| 37122 | 47446149 | 47446846 | 234690 |
| 37123 | 47451919 | 47454263 | 226087, 234690 |
| 37124 | 47492017 | 47492894 | 234690 |
| 37125 | 47496080 | 47498144 | 234690 |
| 37126 | 47498583 | 47500701 | 234690 |
| 37127 | 47994361 | 47995621 | 116062 |
| 37128 | 47998238 | 47999044 | 116062 |
| 37129 | 48009171 | 48010857 | 116062 |
| 37130 | 48131084 | 48135049 | 233230, 138081 |
| 37131 | 48333104 | 48335250 | 230773 |
| 37132 | 48338140 | 48342935 | 230773 |
| 37133 | 48374893 | 48375997 | 230773 |
| 37134 | 48445300 | 48445708 | 230773 |
| 37135 | 48446300 | 48448208 | 230773 |
| 37136 | 48448869 | 48449610 | 230773 |
| 37137 | 48473946 | 48477802 | |
| 37138 | 48505342 | 48506126 | |
| 37139 | 48540620 | 48543697 | 170802 |
| 37140 | 48570843 | 48574319 | 170802 |
| 37141 | 48580185 | 48580768 | 170802 |
| 37142 | 48641242 | 48641793 | |
| 37143 | 48641829 | 48643773 | |
| 37144 | 48645103 | 48648464 | |
| 37145 | 48666819 | 48668895 | 272663, 162869 |
| 37146 | 48735268 | 48736362 | 162869 |
| 37147 | 51000328 | 51001262 | 179915 |
| 37148 | 51240074 | 51240893 | 179915 |
| 37149 | 51464614 | 51465377 | 231918 |
| 37150 | 51470783 | 51472168 | 231918 |
| 37151 | 52623696 | 52625007 | 231918 |
| 37152 | 52727579 | 52728495 | 236837 |
| 37153 | 53146833 | 53147359 | |
| 37154 | 53160499 | 53163269 | |
| 37155 | 53675493 | 53676323 | |
| 37156 | 53725320 | 53725857 | |
| 37157 | 53994010 | 53995816 | 143942, 115239, 270898 |
| 37158 | 54013206 | 54015101 | 068912, 115239, 270898 |
| 37159 | 54053593 | 54054476 | 115239, 270898 |
| 37160 | 54078081 | 54078787 | 115239, 270898 |
| 37161 | 54195415 | 54199220 | 170634, 068878 |
| 37162 | 54214366 | 54215055 | 170634 |
| 37163 | 54341741 | 54343790 | 170634 |
| 37164 | 54370798 | 54371327 | 170634 |
| 37165 | 54439508 | 54440047 | 170634 |
| 37166 | 54556728 | 54558083 | 177994 |
| 37167 | 54681679 | 54685178 | 115306 |
| 37168 | 54698497 | 54699122 | 115306 |
| 37169 | 54719408 | 54719959 | 115306 |
| 37170 | 54759191 | 54761694 | 228108, 115306 |
| 37171 | 54771077 | 54774646 | 234943, 115306 |
| 37172 | 54781967 | 54789648 | 115306 |
| 37173 | 54792263 | 54793095 | 115306 |
| 37174 | 54795790 | 54803416 | 115306 |
| 37175 | 54803600 | 54804601 | 115306 |
| 37176 | 54816102 | 54818319 | 115306 |
| 37177 | 54822928 | 54830941 | 115306 |
| 37178 | 54840188 | 54841509 | 115306 |
| 37179 | 54856267 | 54863355 | 115306 |
| 37180 | 54867930 | 54869760 | 115306 |
| 37181 | 54892524 | 54894564 | 115306 |
| 37182 | 54896782 | 54897596 | 115306 |
| 37183 | 54902775 | 54903376 | |
| 37184 | 54935685 | 54937238 | |
| 37185 | 54949665 | 54951154 | 214595 |
| 37186 | 54977024 | 54977605 | 214595 |
| 37187 | 55085174 | 55085929 | 214595 |
| 37188 | 55107065 | 55107739 | 214595 |
| 37189 | 55130355 | 55132886 | 214595 |
| 37190 | 55135565 | 55136520 | 214595 |
| 37191 | 55275817 | 55279952 | 115310 |
| 37192 | 55360343 | 55362005 | |
| 37193 | 55458728 | 55460837 | 143947, 162994 |
| 37194 | 55495390 | 55497245 | 085760 |
| 37195 | 55508588 | 55510290 | 162997 |
| 37196 | 55645748 | 55647851 | 115355 |
| 37197 | 55746146 | 55747623 | 163001 |
| 37198 | 55782973 | 55783561 | 138041 |
| 37199 | 55836709 | 55837855 | 138041 |
| 37200 | 55843025 | 55846267 | 272606, 138041 |
| 37201 | 55920261 | 55921421 | 138035 |
| 37202 | 56066204 | 56067015 | |
| 37203 | 56102564 | 56103397 | 115380 |
| 37204 | 56112719 | 56113218 | 115380 |
| 37205 | 56674892 | 56677265 | |
| 37206 | 56701381 | 56702340 | |
| 37207 | 56749609 | 56750513 | |
| 37208 | 56990704 | 56991226 | |
| 37209 | 57540696 | 57541333 | |
| 37210 | 58108406 | 58110485 | |
| 37211 | 58273079 | 58274919 | 028116 |
| 37212 | 58467781 | 58468895 | 273063, 115392 |
| 37213 | 58856307 | 58857690 | 233723 |
| 37214 | 60542940 | 60544519 | |
| 37215 | 60592571 | 60593145 | 223929 |
| 37216 | 60642806 | 60644734 | |
| 37217 | 60776797 | 60777903 | 119866 |
| 37218 | 60983098 | 60984374 | 115421 |
| 37219 | 61107449 | 61109800 | 162924, 228414 |
| 37220 | 61153063 | 61153640 | 162924, 267520 |
| 37221 | 61242879 | 61246037 | 162928, 162927 |
| 37222 | 61292781 | 61294018 | 162929 |
| 37223 | 61371856 | 61372765 | 237651, 212978, 162929 |
| 37224 | 61403758 | 61405682 | 173209 |
| 37225 | 61437350 | 61438460 | 115464 |
| 37226 | 61658161 | 61658723 | 115464 |
| 37227 | 61666305 | 61666786 | 115464 |
| 37228 | 61670309 | 61671758 | 115464 |
| 37229 | 61696946 | 61699414 | 270820, 115464 |
| 37230 | 61723712 | 61724335 | 082898 |
| 37231 | 61762765 | 61766613 | 082898 |
| 37232 | 61847522 | 61848310 | |
| 37233 | 61856441 | 61857666 | |
| 37234 | 61885884 | 61887333 | |
| 37235 | 61897142 | 61899633 | |
| 37236 | 61917116 | 61917745 | |
| 37237 | 61921418 | 61922236 | |
| 37238 | 61934905 | 61935665 | |
| 37239 | 61990372 | 61992330 | |
| 37240 | 62080692 | 62082717 | 170264 |
| 37241 | 62114390 | 62116438 | 173163, 115484 |
| 37242 | 62131809 | 62133562 | 173163 |
| 37243 | 62241150 | 62241711 | 173163 |
| 37244 | 62422266 | 62425128 | 170340 |
| 37245 | 62443776 | 62444262 | 170340 |
| 37246 | 62519165 | 62519762 | |
| 37247 | 62520564 | 62521396 | |
| 37248 | 62677441 | 62678683 | |
| 37249 | 62683244 | 62686042 | |
| 37250 | 62687732 | 62690670 | 228541 |
| 37251 | 62694093 | 62695608 | |
| 37252 | 62712361 | 62713907 | |
| 37253 | 62722793 | 62723552 | |
| 37254 | 62728164 | 62728705 | 186889 |
| 37255 | 62732103 | 62734494 | 186889 |
| 37256 | 62795179 | 62799019 | |
| 37257 | 62803357 | 62807800 | |
| 37258 | 62822874 | 62824079 | 226622 |
| 37259 | 62880552 | 62882785 | 226622 |
| 37260 | 62925449 | 62926712 | 115504 |
| 37261 | 62931365 | 62934135 | 115504 |
| 37262 | 63016630 | 63017616 | 115504 |
| 37263 | 63277674 | 63278188 | 115507 |
| 37264 | 63814824 | 63817204 | 014641, 143951 |
| 37265 | 63885303 | 63886126 | 143951 |
| 37266 | 63889491 | 63891128 | 143951 |
| 37267 | 63900868 | 63901687 | 143951 |
| 37268 | 63922665 | 63927335 | 221085, 143951 |
| 37269 | 63939478 | 63940362 | 143951 |
| 37270 | 63950755 | 63951465 | 143951 |
| 37271 | 63956483 | 63957387 | 143951 |
| 37272 | 63961113 | 63966057 | 143951 |
| 37273 | 63967867 | 63969533 | 143951 |
| 37274 | 63982965 | 63983841 | 143951 |
| 37275 | 64015257 | 64020342 | 143951 |
| 37276 | 64046483 | 64049162 | 143951 |
| 37277 | 64053137 | 64056074 | 143951 |
| 37278 | 64066482 | 64071042 | 169764 |
| 37279 | 64080613 | 64081368 | 169764 |
| 37280 | 64089186 | 64093128 | 169764 |
| 37281 | 64098023 | 64099454 | 169764 |
| 37282 | 64220217 | 64220634 | 143952 |
| 37283 | 64244862 | 64248093 | 143952 |
| 37284 | 64250904 | 64252386 | |
| 37285 | 64289969 | 64290561 | |
| 37286 | 64370490 | 64371830 | 197329, 225889 |
| 37287 | 64384902 | 64385503 | 225889 |
| 37288 | 64419958 | 64420295 | 225889, 230923 |
| 37289 | 64470090 | 64471428 | 225889 |
| 37290 | 64487691 | 64490504 | |
| 37291 | 64499830 | 64501796 | |
| 37292 | 64617866 | 64620093 | |
| 37293 | 64627240 | 64627731 | 223935 |
| 37294 | 64630420 | 64632290 | 223935 |
| 37295 | 64632735 | 64634098 | 223935 |
| 37296 | 64680152 | 64682444 | 119862, 223935 |
| 37297 | 64749663 | 64752796 | 119844, 260101 |
| 37298 | 64819580 | 64823628 | 119844 |
| 37299 | 64832835 | 64834671 | 233694 |
| 37300 | 64835889 | 64837410 | 233694 |
| 37301 | 64861390 | 64861938 | 179833 |
| 37302 | 64873228 | 64874144 | 226756, 179833 |
| 37303 | 64878572 | 64883510 | 179833 |
| 37304 | 64902813 | 64903556 | 179833 |
| 37305 | 64912352 | 64913981 | 179833 |
| 37306 | 64915830 | 64916774 | 179833 |
| 37307 | 64924059 | 64927550 | 179833 |
| 37308 | 64956995 | 64959755 | 179833 |
| 37309 | 64976559 | 64979583 | 179833 |
| 37310 | 64993721 | 64996222 | |
| 37311 | 65058221 | 65059763 | |
| 37312 | 65086193 | 65087494 | 234572 |
| 37313 | 65091697 | 65093252 | |
| 37314 | 65115982 | 65116453 | |
| 37315 | 65125882 | 65127204 | |
| 37316 | 65149648 | 65150585 | |
| 37317 | 65164538 | 65165671 | |
| 37318 | 65170671 | 65171421 | |
| 37319 | 65201793 | 65203038 | |
| 37320 | 65214760 | 65217510 | 115902 |
| 37321 | 65233637 | 65234203 | 115902 |
| 37322 | 65255361 | 65256192 | |
| 37323 | 65282874 | 65284505 | 011523 |
| 37324 | 65291888 | 65292671 | 011523 |
| 37325 | 65296791 | 65298687 | 138069, 011523 |
| 37326 | 65307857 | 65308838 | 011523, 138069 |
| 37327 | 65355541 | 65358587 | 138069 |
| 37328 | 65397682 | 65398196 | |
| 37329 | 65453710 | 65456185 | 138071 |
| 37330 | 65527250 | 65528068 | |
| 37331 | 65543070 | 65544303 | 198369 |
| 37332 | 65546022 | 65548604 | 198369 |
| 37333 | 65590251 | 65591834 | 198369 |
| 37334 | 65600049 | 65601362 | 232693, 198369 |
| 37335 | 65604612 | 65605600 | 198369, 232693 |
| 37336 | 65606610 | 65607649 | 198369, 232693 |
| 37337 | 65609706 | 65611102 | 198369 |
| 37338 | 65617173 | 65617700 | 198369 |
| 37339 | 65622873 | 65624600 | 198369 |
| 37340 | 65629432 | 65630306 | 198369 |
| 37341 | 65644026 | 65644600 | 198369 |
| 37342 | 65656957 | 65660499 | 198369 |
| 37343 | 65662478 | 65665219 | 204929 |
| 37344 | 65754616 | 65755802 | 204929 |
| 37345 | 65772344 | 65773701 | 243029, 204929 |
| 37346 | 65803235 | 65805279 | 204929 |
| 37347 | 65831143 | 65832448 | 235725, 204929 |
| 37348 | 66149806 | 66150528 | 204929 |
| 37349 | 66659824 | 66663516 | 143995, 226819 |
| 37350 | 66721768 | 66724212 | 143995 |
| 37351 | 66758210 | 66758718 | 143995 |
| 37352 | 66788886 | 66789964 | 143995 |
| 37353 | 66790517 | 66791095 | 143995 |
| 37354 | 66801613 | 66803093 | 232046 |
| 37355 | 67264787 | 67265336 | |
| 37356 | 67267076 | 67267977 | 223859 |
| 37357 | 67616185 | 67617277 | |
| 37358 | 67624184 | 67625509 | 143971 |
| 37359 | 67627630 | 67628931 | 143971 |
| 37360 | 68289249 | 68290910 | 197223 |
| 37361 | 68342925 | 68343773 | |
| 37362 | 68348144 | 68348716 | |
| 37363 | 68383738 | 68385924 | 115946, 243667, 273398 |
| 37364 | 68477708 | 68480937 | 273064, 221823, 273398 |
| 37365 | 68653631 | 68656000 | |
| 37366 | 68658163 | 68659280 | |
| 37367 | 68667652 | 68669305 | |
| 37368 | 68694019 | 68695523 | 169621, 204923 |
| 37369 | 68821184 | 68822759 | 169621 |
| 37370 | 69056280 | 69056756 | |
| 37371 | 69134402 | 69136870 | |
| 37372 | 69144032 | 69145092 | |
| 37373 | 69147231 | 69148461 | |
| 37374 | 69163250 | 69163969 | |
| 37375 | 69289287 | 69290112 | 169604 |
| 37376 | 69404899 | 69405801 | 169604 |
| 37377 | 69518286 | 69518932 | |
| 37378 | 69613302 | 69615182 | 198380 |
| 37379 | 69663425 | 69665479 | 169599 |
| 37380 | 69727130 | 69727572 | 115977 |
| 37381 | 69765849 | 69767000 | 115977 |
| 37382 | 69808635 | 69809417 | 115977 |
| 37383 | 69815722 | 69816522 | 115977 |
| 37384 | 69869072 | 69872244 | 196975, 115977 |
| 37385 | 69900939 | 69901850 | 115977, 196975 |
| 37386 | 69902465 | 69902918 | 196975 |
| 37387 | 69922268 | 69923218 | 196975 |
| 37388 | 69967774 | 69970217 | 196975 |
| 37389 | 69983118 | 69983886 | 196975 |
| 37390 | 69995969 | 69998247 | 196975 |
| 37391 | 70005864 | 70009869 | 196975 |
| 37392 | 70010819 | 70012113 | 196975 |
| 37393 | 70016213 | 70016746 | 196975 |
| 37394 | 70024979 | 70025627 | 196975 |
| 37395 | 70042733 | 70044671 | 196975 |
| 37396 | 70055728 | 70057911 | 087338 |
| 37397 | 70120338 | 70122251 | 124380 |
| 37398 | 70140655 | 70143293 | 059728 |
| 37399 | 70145007 | 70145883 | 059728 |
| 37400 | 70205598 | 70206207 | 179818 |
| 37401 | 70207426 | 70207874 | 179818 |
| 37402 | 70294818 | 70297314 | 179818 |
| 37403 | 70309882 | 70317146 | 169564, 233060, 179818 |
| 37404 | 70324815 | 70325866 | 226505 |
| 37405 | 70334907 | 70336459 | |
| 37406 | 70350876 | 70353030 | 231327 |
| 37407 | 70362860 | 70363774 | |
| 37408 | 70367691 | 70371154 | |
| 37409 | 70415102 | 70416563 | 115998 |
| 37410 | 70416675 | 70418580 | 115998 |
| 37411 | 70474231 | 70476796 | 116001, 115998 |
| 37412 | 70485047 | 70486091 | 116005 |
| 37413 | 70496667 | 70497317 | 116005 |
| 37414 | 70519692 | 70521617 | 143977 |
| 37415 | 70528071 | 70529494 | 233849, 035141 |
| 37416 | 70530908 | 70531397 | |
| 37417 | 70741771 | 70742345 | 163235 |
| 37418 | 70751349 | 70752693 | 163235 |
| 37419 | 70779817 | 70782080 | 163235 |
| 37420 | 71221018 | 71222931 | 228384, 144043, 258881 |
| 37421 | 71294382 | 71296393 | 272735, 124357 |
| 37422 | 71356521 | 71358544 | 124383, 124370 |
| 37423 | 71452986 | 71454833 | 124374 |
| 37424 | 71503645 | 71504270 | 075292 |
| 37425 | 71504525 | 71505229 | 075292 |
| 37426 | 71558437 | 71559956 | 075292 |
| 37427 | 71563836 | 71564559 | 075292 |
| 37428 | 71605694 | 71606725 | 075292 |
| 37429 | 71612027 | 71612539 | 075292 |
| 37430 | 71615108 | 71615798 | 075292 |
| 37431 | 71679713 | 71681745 | 135636 |
| 37432 | 71683031 | 71684208 | 135636 |
| 37433 | 71693279 | 71694520 | 135636 |
| 37434 | 71704413 | 71705056 | 135636 |
| 37435 | 71757896 | 71759296 | 135636 |
| 37436 | 71782794 | 71783826 | 135636 |
| 37437 | 71842195 | 71843091 | 135636 |
| 37438 | 71943849 | 71944836 | |
| 37439 | 72011455 | 72012586 | |
| 37440 | 72057612 | 72059218 | |
| 37441 | 72107752 | 72109188 | |
| 37442 | 72109803 | 72111983 | |
| 37443 | 72161634 | 72162826 | |
| 37444 | 72167344 | 72168519 | |
| 37445 | 72175630 | 72176682 | |
| 37446 | 72177334 | 72178891 | |
| 37447 | 72179424 | 72180426 | |
| 37448 | 72304584 | 72305503 | |
| 37449 | 72365383 | 72366030 | 003137 |
| 37450 | 72463048 | 72464050 | 144036 |
| 37451 | 72482552 | 72483178 | 144036 |
| 37452 | 72775021 | 72775664 | 144036 |
| 37453 | 72921871 | 72922667 | 144036 |
| 37454 | 72949735 | 72951420 | 144036 |
| 37455 | 72982526 | 72985026 | 144036 |
| 37456 | 73005498 | 73006543 | 144036 |
| 37457 | 73052221 | 73053503 | 144036 |
| 37458 | 73113746 | 73115430 | 116096 |
| 37459 | 73142994 | 73143936 | 135638 |
| 37460 | 73206858 | 73210455 | 144040 |
| 37461 | 73212884 | 73213833 | 144040 |
| 37462 | 73226524 | 73229541 | 144040 |
| 37463 | 73257964 | 73260286 | 144040 |
| 37464 | 73260438 | 73261477 | 144040 |
| 37465 | 73296423 | 73299743 | 144040 |
| 37466 | 73316442 | 73317198 | 135631 |
| 37467 | 73338309 | 73341022 | 272702, 135631 |
| 37468 | 73402691 | 73405145 | |
| 37469 | 73441064 | 73442301 | 135632 |
| 37470 | 73459887 | 73462704 | 135624, 135617 |
| 37471 | 73612573 | 73614143 | 116127 |
| 37472 | 73671190 | 73671779 | 116127 |
| 37473 | 73852519 | 73853343 | |
| 37474 | 73869554 | 73870251 | |
| 37475 | 73912868 | 73913779 | |
| 37476 | 73963754 | 73965410 | 144034 |
| 37477 | 74006251 | 74007580 | 144048 |
| 37478 | 74086818 | 74087728 | 124356 |
| 37479 | 74153545 | 74155351 | 114956 |
| 37480 | 74207874 | 74209341 | 237883 |
| 37481 | 74210477 | 74213572 | 235499 |
| 37482 | 74374099 | 74375420 | 225439, 163170 |
| 37483 | 74405061 | 74406551 | 114978 |
| 37484 | 74494214 | 74494749 | 264324, 188687 |
| 37485 | 74610218 | 74610815 | 204843 |
| 37486 | 74618112 | 74619840 | 204843, 237737 |
| 37487 | 74651827 | 74652331 | 005448 |
| 37488 | 74656405 | 74657810 | 114993 |
| 37489 | 74667134 | 74669955 | 114993 |
| 37490 | 74671209 | 74671853 | |
| 37491 | 74674223 | 74675435 | |
| 37492 | 74681572 | 74682862 | 115274 |
| 37493 | 74684156 | 74686544 | 239779, 115274 |
| 37494 | 74691360 | 74693145 | 115275 |
| 37495 | 74698539 | 74700631 | 204822, 135637 |
| 37496 | 74709044 | 74711013 | 115282, 135637 |
| 37497 | 74715286 | 74716739 | 115282 |
| 37498 | 74718984 | 74719655 | 115282 |
| 37499 | 74728886 | 74731939 | 257702, 179528, 272183 |
| 37500 | 74733669 | 74735708 | 115289 |
| 37501 | 74755691 | 74758049 | 115317, 115307 |
| 37502 | 74775776 | 74776464 | 115325, 115318 |
| 37503 | 75185125 | 75186332 | 115350 |
| 37504 | 75743314 | 75744575 | 115363 |
| 37505 | 75744623 | 75747253 | 115363 |
| 37506 | 75748500 | 75749090 | 115363 |
| 37507 | 75749575 | 75751281 | 231172, 115363 |
| 37508 | 75787078 | 75788980 | 115363 |
| 37509 | 75792085 | 75793897 | 115363 |
| 37510 | 75867602 | 75868902 | |
| 37511 | 75873235 | 75875162 | 115364 |
| 37512 | 75935943 | 75938799 | 005436 |
| 37513 | 76092149 | 76092672 | |
| 37514 | 76744571 | 76745049 | |
| 37515 | 77792930 | 77795366 | 176204 |
| 37516 | 78336343 | 78336822 | 229494 |
| 37517 | 83016194 | 83018583 | |
| 37518 | 83478086 | 83479004 | |
| 37519 | 83509286 | 83510184 | |
| 37520 | 83704936 | 83705767 | |
| 37521 | 83750038 | 83750457 | 232548 |
| 37522 | 84520835 | 84521945 | |
| 37523 | 84685523 | 84687510 | 163541 |
| 37524 | 84859038 | 84859551 | 115423 |
| 37525 | 85107609 | 85108568 | 186854 |
| 37526 | 85131862 | 85133836 | 034510, 186854 |
| 37527 | 85146299 | 85148148 | |
| 37528 | 85152716 | 85153546 | |
| 37529 | 85172693 | 85174008 | |
| 37530 | 85184624 | 85187380 | |
| 37531 | 85189732 | 85192401 | |
| 37532 | 85196122 | 85200935 | 176407 |
| 37533 | 85359825 | 85361708 | 152284 |
| 37534 | 85370361 | 85371392 | 152284 |
| 37535 | 85496864 | 85497935 | 152284 |
| 37536 | 85521445 | 85522192 | 152284 |
| 37537 | 85533836 | 85534825 | 152284 |
| 37538 | 85551429 | 85552598 | 152291 |
| 37539 | 85552630 | 85556051 | 152291 |
| 37540 | 85580577 | 85582790 | 115459, 042445 |
| 37541 | 85644901 | 85647358 | 152292, 042493 |
| 37542 | 85647446 | 85650070 | 152292 |
| 37543 | 85652618 | 85653264 | 152292 |
| 37544 | 85663465 | 85666512 | 152292 |
| 37545 | 85671753 | 85674936 | |
| 37546 | 85694851 | 85696687 | |
| 37547 | 85697697 | 85698640 | |
| 37548 | 85702952 | 85703795 | |
| 37549 | 85726110 | 85726811 | |
| 37550 | 85726974 | 85727713 | |
| 37551 | 85743643 | 85744624 | |
| 37552 | 85764534 | 85767951 | 168906 |
| 37553 | 85772389 | 85775242 | 168906, 115486 |
| 37554 | 85786493 | 85789521 | 118640, 115486 |
| 37555 | 85804369 | 85806088 | 118640 |
| 37556 | 85810647 | 85813316 | 168899 |
| 37557 | 85821748 | 85823960 | 168894 |
| 37558 | 85828680 | 85830792 | 168883, 168890 |
| 37559 | 85838198 | 85839681 | 168887, 168883 |
| 37560 | 85842988 | 85844036 | 168883 |
| 37561 | 85889640 | 85890281 | 168878 |
| 37562 | 85907236 | 85909295 | |
| 37563 | 85931769 | 85939156 | |
| 37564 | 85939250 | 85941798 | |
| 37565 | 85955608 | 85956791 | |
| 37566 | 85963588 | 85966084 | |
| 37567 | 85967711 | 85972238 | |
| 37568 | 85980780 | 85983466 | 168874 |
| 37569 | 85983792 | 85984674 | 168874 |
| 37570 | 85997494 | 86000672 | 168874 |
| 37571 | 86006570 | 86007201 | 168874 |
| 37572 | 86019662 | 86022632 | |
| 37573 | 86025844 | 86027307 | |
| 37574 | 86033334 | 86034407 | |
| 37575 | 86114672 | 86118086 | 232504, 115525 |
| 37576 | 86138897 | 86140386 | |
| 37577 | 86140892 | 86142449 | |
| 37578 | 86162547 | 86165052 | |
| 37579 | 86172038 | 86172995 | |
| 37580 | 86176025 | 86177088 | |
| 37581 | 86178747 | 86180322 | |
| 37582 | 86244946 | 86245610 | |
| 37583 | 86249993 | 86250537 | 068654 |
| 37584 | 86288840 | 86290086 | 068654 |
| 37585 | 86299360 | 86300051 | 068654 |
| 37586 | 86306914 | 86308148 | 068654 |
| 37587 | 86332522 | 86334390 | 132300, 068654 |
| 37588 | 86421725 | 86423603 | 273080, 132305 |
| 37589 | 86425993 | 86427519 | 132313 |
| 37590 | 86620709 | 86621510 | |
| 37591 | 86623273 | 86624020 | |
| 37592 | 86667354 | 86669866 | 115548 |
| 37593 | 86673158 | 86673637 | 115548 |
| 37594 | 86703935 | 86704942 | 115548 |
| 37595 | 86789279 | 86791458 | 249884, 228363, 115561 |
| 37596 | 86842701 | 86843095 | 249884, 228363, 239305, |
| | | | 115561 |
| 37597 | 86849008 | 86851794 | 239305, 249884, 115561 |
| 37598 | 86854934 | 86856001 | 249884, 115561 |
| 37599 | 86858714 | 86860834 | 249884, 115561 |
| 37600 | 86864290 | 86864864 | 249884, 115561 |
| 37601 | 86876121 | 86877059 | 249884, 115561 |
| 37602 | 86898340 | 86899853 | 249884, 115561 |
| 37603 | 86931331 | 86932372 | 249884, 115561 |
| 37604 | 87034664 | 87036006 | 153563 |
| 37605 | 88315149 | 88317006 | 222724 |
| 37606 | 88353950 | 88356231 | 172086 |
| 37607 | 88399809 | 88400515 | 115593 |
| 37608 | 88426674 | 88428680 | 163586 |
| 37609 | 88429613 | 88430623 | |
| 37610 | 88436015 | 88436911 | |
| 37611 | 88438154 | 88439848 | |
| 37612 | 88443989 | 88445625 | |
| 37613 | 88469350 | 88471221 | 144115 |
| 37614 | 88478380 | 88479527 | 144115 |
| 37615 | 88620971 | 88621576 | |
| 37616 | 88899212 | 88901088 | 172071 |
| 37617 | 88926017 | 88928379 | 234028, 172071 |
| 37618 | 88957886 | 88959566 | |
| 37619 | 88965764 | 88967519 | |
| 37620 | 88968068 | 88969593 | |
| 37621 | 88990342 | 88992111 | 153574 |
| 37622 | 91669804 | 91671231 | |
| 37623 | 95786687 | 95788126 | 144029 |
| 37624 | 95830508 | 95832466 | 163067, 144026 |
| 37625 | 95872749 | 95873938 | 233757 |
| 37626 | 95933488 | 95934108 | |
| 37627 | 95971210 | 95972257 | 115041 |
| 37628 | 96011165 | 96012391 | 115041 |
| 37629 | 96055844 | 96057193 | |
| 37630 | 96057371 | 96058078 | |
| 37631 | 96067800 | 96069214 | 115042 |
| 37632 | 96191931 | 96193317 | 272913, 229689 |
| 37633 | 96343108 | 96343709 | |
| 37634 | 96347482 | 96350074 | |
| 37635 | 96360796 | 96362078 | |
| 37636 | 96810353 | 96811444 | 228873, 158050 |
| 37637 | 96872718 | 96875366 | 204685, 084090 |
| 37638 | 96917331 | 96917810 | 135956 |
| 37639 | 96925076 | 96927864 | 135956 |
| 37640 | 96930542 | 96932628 | 144021, 135956 |
| 37641 | 96970370 | 96972310 | 144028 |
| 37642 | 96986842 | 96987867 | 230747 |
| 37643 | 97007580 | 97008696 | 121152 |
| 37644 | 97075262 | 97076163 | |
| 37645 | 97088947 | 97089943 | |
| 37646 | 97093024 | 97094244 | |
| 37647 | 97151174 | 97152594 | |
| 37648 | 97170474 | 97172045 | 163121 |
| 37649 | 97173686 | 97174306 | 163121 |
| 37650 | 97197599 | 97198079 | 235480 |
| 37651 | 97202287 | 97203602 | 196843 |
| 37652 | 97215616 | 97217827 | 196843 |
| 37653 | 97221879 | 97224727 | |
| 37654 | 97267261 | 97268698 | 114982 |
| 37655 | 97270734 | 97275539 | 114982 |
| 37656 | 97281838 | 97285938 | 114982 |
| 37657 | 97296212 | 97298836 | 114982 |
| 37658 | 97301511 | 97305017 | 114982 |
| 37659 | 97330519 | 97331310 | 249715 |
| 37660 | 97426153 | 97427584 | 158158 |
| 37661 | 97452571 | 97453370 | 158158 |
| 37662 | 97453626 | 97454949 | 158158 |
| 37663 | 97480965 | 97483091 | 168763 |
| 37664 | 97484662 | 97485551 | 168763 |
| 37665 | 97498361 | 97499171 | 168763, 163126 |
| 37666 | 97520444 | 97520992 | 213337, 163126 |
| 37667 | 97522774 | 97525138 | 213337, 163126 |
| 37668 | 97527263 | 97528542 | 168758 |
| 37669 | 97533431 | 97536379 | 168758 |
| 37670 | 98261540 | 98263339 | 135940 |
| 37671 | 98278673 | 98281604 | 201806, 228486, 115073 |
| 37672 | 98461598 | 98462743 | 075568 |
| 37673 | 98485641 | 98488180 | 075568 |
| 37674 | 98552051 | 98552503 | 075568 |
| 37675 | 98611066 | 98613192 | 075568 |
| 37676 | 98701196 | 98701847 | |
| 37677 | 99060864 | 99062471 | 040933 |
| 37678 | 99073078 | 99074722 | 040933 |
| 37679 | 99224131 | 99226606 | 115446, 183513 |
| 37680 | 99317513 | 99319517 | 071073 |
| 37681 | 99321087 | 99321975 | 071073 |
| 37682 | 99346564 | 99348904 | 071073 |
| 37683 | 99420028 | 99421076 | 196872 |
| 37684 | 99459010 | 99459737 | 196872 |
| 37685 | 99756835 | 99758350 | 273045, 241962, 135951 |
| 37686 | 99770068 | 99772680 | 273155, 144182, 135951, |
| | | | 241962 |
| 37687 | 99796667 | 99798806 | 185414, 158411, 241962, |
| | | | 273155 |
| 37688 | 99951664 | 99955113 | 158417, 115514 |
| 37689 | 100019492 | 100020465 | 135945 |
| 37690 | 100104889 | 100107315 | 135945 |
| 37691 | 100252777 | 100253603 | 144218 |
| 37692 | 100466531 | 100468026 | 144218 |
| 37693 | 100468072 | 100470223 | 144218 |
| 37694 | 100515227 | 100516614 | 144218 |
| 37695 | 100629533 | 100631487 | 144218 |
| 37696 | 100684127 | 100685162 | 144218 |
| 37697 | 100685690 | 100687090 | 144218 |
| 37698 | 100721247 | 100722268 | 230393, 144218 |
| 37699 | 100740680 | 100742054 | 144218 |
| 37700 | 100830057 | 100831736 | 232084 |
| 37701 | 100901988 | 100903760 | 170500 |
| 37702 | 100907570 | 100908911 | 170500 |
| 37703 | 100937870 | 100939048 | 170500 |
| 37704 | 101178713 | 101180377 | 115539 |
| 37705 | 101204483 | 101204986 | |
| 37706 | 101349688 | 101350959 | |
| 37707 | 101358398 | 101359797 | |
| 37708 | 101360919 | 101362783 | |
| 37709 | 101368405 | 101372291 | |
| 37710 | 101396563 | 101397622 | |
| 37711 | 101407783 | 101408796 | |
| 37712 | 101430753 | 101431651 | |
| 37713 | 101434629 | 101438262 | 170485 |
| 37714 | 101438370 | 101439867 | 232034, 170485 |
| 37715 | 101449275 | 101450087 | 232034, 170485 |
| 37716 | 101459366 | 101460373 | 232034, 170485 |
| 37717 | 101491516 | 101493100 | 170485 |
| 37718 | 101499040 | 101501053 | 170485 |
| 37719 | 101539928 | 101542477 | 170485 |
| 37720 | 101557094 | 101558241 | 170485 |
| 37721 | 101574399 | 101574920 | 170485 |
| 37722 | 101614588 | 101615385 | 223947 |
| 37723 | 101617778 | 101620030 | 071082, 223947 |
| 37724 | 101680411 | 101682131 | 204634 |
| 37725 | 101703225 | 101704005 | 204634 |
| 37726 | 101705796 | 101706320 | 204634 |
| 37727 | 101722933 | 101723561 | 204634 |
| 37728 | 101726816 | 101727664 | 204634 |
| 37729 | 101730030 | 101730842 | 204634 |
| 37730 | 101733313 | 101734274 | 204634 |
| 37731 | 101735359 | 101738469 | 204634 |
| 37732 | 101739738 | 101743244 | 204634 |
| 37733 | 101751843 | 101752549 | 204634 |
| 37734 | 101766056 | 101769782 | 272902, 204634 |
| 37735 | 101792401 | 101793038 | 204634 |
| 37736 | 101846581 | 101849357 | 204634 |
| 37737 | 101868209 | 101870509 | 158435, 204634 |
| 37738 | 101903740 | 101904211 | 163162 |
| 37739 | 101922729 | 101926022 | 264857, 163162 |
| 37740 | 102085953 | 102087039 | 196460 |
| 37741 | 102132774 | 102135999 | |
| 37742 | 102183059 | 102183833 | |
| 37743 | 102212486 | 102213418 | |
| 37744 | 102213572 | 102215166 | |
| 37745 | 102241655 | 102242474 | |
| 37746 | 102246592 | 102247280 | |
| 37747 | 102305720 | 102306584 | |
| 37748 | 102312957 | 102317246 | 071054 |
| 37749 | 102351330 | 102352140 | 071054 |
| 37750 | 102352264 | 102355612 | 071054 |
| 37751 | 102388344 | 102388771 | 071054 |
| 37752 | 102405003 | 102406418 | 071054 |
| 37753 | 102419592 | 102420136 | 071054 |
| 37754 | 102420171 | 102420989 | 071054 |
| 37755 | 102432168 | 102432969 | 071054 |
| 37756 | 102443609 | 102444606 | 071054 |
| 37757 | 102473715 | 102476060 | 071054 |
| 37758 | 102505306 | 102506637 | 071054 |
| 37759 | 102577006 | 102579308 | |
| 37760 | 102603398 | 102604040 | |
| 37761 | 102614577 | 102619448 | 115590 |
| 37762 | 102643007 | 102645453 | 115590 |
| 37763 | 102647104 | 102648223 | |
| 37764 | 102665846 | 102667812 | |
| 37765 | 102672029 | 102673287 | |
| 37766 | 102673420 | 102675342 | |
| 37767 | 102678926 | 102682863 | 115594 |
| 37768 | 102685462 | 102687684 | 115594 |
| 37769 | 102711347 | 102712410 | 115594 |
| 37770 | 102715711 | 102718992 | 115594 |
| 37771 | 102720216 | 102727957 | 115594 |
| 37772 | 102742967 | 102745641 | 115594 |
| 37773 | 102758354 | 102759990 | 115594 |
| 37774 | 102771501 | 102773083 | 115594 |
| 37775 | 102803204 | 102804973 | 115598 |
| 37776 | 102806423 | 102807341 | 115598 |
| 37777 | 102808640 | 102810010 | 115598 |
| 37778 | 102843148 | 102845132 | 115598 |
| 37779 | 102856649 | 102857803 | |
| 37780 | 102867412 | 102868475 | |
| 37781 | 102874187 | 102876010 | |
| 37782 | 102971931 | 102973267 | 115604 |
| 37783 | 103352029 | 103353604 | 170417, 135953 |
| 37784 | 103417669 | 103419206 | 170417 |
| 37785 | 104389790 | 104391328 | |
| 37786 | 104404618 | 104405388 | |
| 37787 | 104405408 | 104406635 | |
| 37788 | 105029920 | 105030858 | 225765, 227157 |
| 37789 | 105036842 | 105037432 | |
| 37790 | 105042179 | 105043919 | |
| 37791 | 105110197 | 105110732 | 235597, 234781 |
| 37792 | 105556021 | 105556639 | 224509 |
| 37793 | 105653876 | 105655401 | 135972, 224509 |
| 37794 | 105945054 | 105947197 | 135966, 235319 |
| 37795 | 105952923 | 105955012 | 135974, 272994 |
| 37796 | 106012503 | 106013832 | 115641 |
| 37797 | 106014126 | 106016168 | 115641 |
| 37798 | 106019872 | 106021649 | 115641 |
| 37799 | 106025976 | 106027504 | 115641 |
| 37800 | 106037442 | 106038039 | 115641 |
| 37801 | 106070986 | 106073099 | |
| 37802 | 106076541 | 106077417 | |
| 37803 | 106106206 | 106107079 | |
| 37804 | 106107193 | 106108180 | |
| 37805 | 106134160 | 106134953 | |
| 37806 | 106328442 | 106330629 | |
| 37807 | 106361327 | 106362246 | 071051 |
| 37808 | 106543314 | 106544023 | |
| 37809 | 106646942 | 106648069 | |
| 37810 | 106743420 | 106744477 | 115652 |
| 37811 | 106809119 | 106811431 | 115652 |
| 37812 | 106885831 | 106887462 | |
| 37813 | 106921006 | 106921737 | 233939 |
| 37814 | 107196096 | 107196721 | |
| 37815 | 108815905 | 108816685 | 225328 |
| 37816 | 108883670 | 108884876 | |
| 37817 | 109039702 | 109040355 | 271284 |
| 37818 | 109064610 | 109066672 | 135968 |
| 37819 | 109108719 | 109109303 | 135968 |
| 37820 | 109149469 | 109152044 | 169756, 214184 |
| 37821 | 109252738 | 109254160 | 169756 |
| 37822 | 109335041 | 109336477 | 153201 |
| 37823 | 109402762 | 109403632 | 163006 |
| 37824 | 109521050 | 109522390 | 135960 |
| 37825 | 109573511 | 109574243 | 135960 |
| 37826 | 109637952 | 109638731 | |
| 37827 | 109647505 | 109648634 | |
| 37828 | 109834588 | 109835811 | 172985 |
| 37829 | 109858610 | 109860200 | 172985 |
| 37830 | 110069796 | 110070418 | 172985 |
| 37831 | 110314647 | 110320949 | 186522 |
| 37832 | 110362245 | 110362669 | 230696, 186522 |
| 37833 | 110370274 | 110374122 | 198142, 186522 |
| 37834 | 110387621 | 110390521 | |
| 37835 | 110393560 | 110394396 | |
| 37836 | 110395392 | 110396347 | |
| 37837 | 110401773 | 110402971 | |
| 37838 | 110448076 | 110449597 | |
| 37839 | 110451533 | 110452378 | |
| 37840 | 110459171 | 110461128 | |
| 37841 | 110962273 | 110962909 | 144061 |
| 37842 | 110969244 | 110970450 | 175701 |
| 37843 | 111474465 | 111475647 | |
| 37844 | 111477839 | 111478647 | |
| 37845 | 111789197 | 111791828 | 153093 |
| 37846 | 111877373 | 111881395 | 153094 |
| 37847 | 111917645 | 111920051 | 153094 |
| 37848 | 111933234 | 111937834 | |
| 37849 | 111942271 | 111943991 | |
| 37850 | 111979292 | 111979712 | 172965 |
| 37851 | 111984813 | 111985939 | 172965 |
| 37852 | 112279646 | 112281157 | |
| 37853 | 112374861 | 112376790 | |
| 37854 | 112416319 | 112418463 | |
| 37855 | 112428848 | 112429689 | |
| 37856 | 112430920 | 112431626 | |
| 37857 | 112455436 | 112457704 | |
| 37858 | 112463382 | 112464943 | |
| 37859 | 112473501 | 112473993 | |
| 37860 | 112640949 | 112642684 | 153107 |
| 37861 | 112655352 | 112657045 | 153208 |
| 37862 | 112666243 | 112666946 | 153208 |
| 37863 | 112670106 | 112670677 | 153208 |
| 37864 | 112677112 | 112678802 | 153208 |
| 37865 | 112688420 | 112688847 | 153208 |
| 37866 | 112703487 | 112704878 | 153208 |
| 37867 | 112730356 | 112731712 | 153208 |
| 37868 | 112812610 | 112814257 | 153214 |
| 37869 | 113011633 | 113012836 | 144161 |
| 37870 | 113016800 | 113017558 | |
| 37871 | 113032234 | 113034760 | 188177 |
| 37872 | 113072051 | 113073016 | 188177 |
| 37873 | 113073934 | 113075991 | 188177 |
| 37874 | 113238486 | 113241603 | 114999 |
| 37875 | 113298972 | 113301002 | 125630, 114999 |
| 37876 | 113336816 | 113337301 | 207383 |
| 37877 | 113341033 | 113343294 | 125611 |
| 37878 | 113357509 | 113358430 | 243389 |
| 37879 | 113363712 | 113364863 | 243389 |
| 37880 | 113379866 | 113380499 | |
| 37881 | 113401029 | 113404936 | 144136, 237753 |
| 37882 | 113438169 | 113438907 | |
| 37883 | 113448339 | 113451176 | |
| 37884 | 113494398 | 113495829 | 169607, 144130 |
| 37885 | 113547783 | 113548297 | |
| 37886 | 113550892 | 113552985 | |
| 37887 | 113568937 | 113570297 | |
| 37888 | 113575526 | 113576279 | |
| 37889 | 113581668 | 113582679 | |
| 37890 | 113591896 | 113592441 | 125538 |
| 37891 | 113696518 | 113697717 | |
| 37892 | 113715942 | 113717049 | |
| 37893 | 113845751 | 113846745 | |
| 37894 | 113881447 | 113886278 | 136689 |
| 37895 | 113913658 | 113917649 | 125637 |
| 37896 | 113921930 | 113923255 | 125637 |
| 37897 | 113924247 | 113926425 | 125637 |
| 37898 | 113942902 | 113943584 | 125637 |
| 37899 | 113955529 | 113958506 | 125637 |
| 37900 | 113963853 | 113965013 | 125637 |
| 37901 | 113975067 | 113977255 | 125618, 189223 |
| 37902 | 113996776 | 114000607 | 258395, 125618, 189223 |
| 37903 | 114002675 | 114003770 | 125618, 189223 |
| 37904 | 114006556 | 114007696 | 125618, 189223 |
| 37905 | 114038954 | 114042861 | |
| 37906 | 114081555 | 114088909 | |
| 37907 | 114093523 | 114098138 | |
| 37908 | 114127715 | 114128582 | |
| 37909 | 114464682 | 114465139 | 115084 |
| 37910 | 114512991 | 114515084 | 115084 |
| 37911 | 114646471 | 114649628 | 115091, 270019 |
| 37912 | 114651808 | 114652551 | 115091 |
| 37913 | 114917705 | 114918567 | |
| 37914 | 117207217 | 117209567 | |
| 37915 | 118571847 | 118573456 | 088205 |
| 37916 | 118593383 | 118593980 | 236255 |
| 37917 | 118613945 | 118614478 | |
| 37918 | 118674753 | 118675528 | 125633, 125631 |
| 37919 | 118737338 | 118739671 | 125633 |
| 37920 | 118770344 | 118772774 | 125633 |
| 37921 | 118842189 | 118843758 | |
| 37922 | 118845016 | 118848149 | 125629 |
| 37923 | 118869453 | 118872123 | |
| 37924 | 118879428 | 118882645 | |
| 37925 | 118903301 | 118905220 | 226856 |
| 37926 | 118936948 | 118937486 | 226856 |
| 37927 | 118942538 | 118944793 | 226856 |
| 37928 | 119076703 | 119077413 | |
| 37929 | 119079112 | 119079732 | |
| 37930 | 119187597 | 119189445 | |
| 37931 | 119612866 | 119613723 | |
| 37932 | 119625623 | 119626518 | |
| 37933 | 119636096 | 119636655 | |
| 37934 | 119951939 | 119953197 | |
| 37935 | 119975294 | 119976854 | |
| 37936 | 119979103 | 119979722 | |
| 37937 | 119979896 | 119983453 | 115107 |
| 37938 | 119997534 | 119999238 | 115107 |
| 37939 | 120013332 | 120013961 | 115107 |
| 37940 | 120040917 | 120042550 | |
| 37941 | 120123412 | 120125841 | 155368, 186132 |
| 37942 | 120144805 | 120145725 | |
| 37943 | 120160137 | 120160954 | |
| 37944 | 120183307 | 120185124 | |
| 37945 | 120188414 | 120193683 | 171227 |
| 37946 | 120239734 | 120241912 | 231013, 080293 |
| 37947 | 120242243 | 120250985 | 231013, 080293 |
| 37948 | 120301496 | 120303273 | 163075 |
| 37949 | 120310727 | 120312306 | 163075 |
| 37950 | 120336923 | 120338125 | 163075 |
| 37951 | 120435290 | 120437729 | 144120 |
| 37952 | 120478601 | 120479867 | |
| 37953 | 120501710 | 120502443 | |
| 37954 | 120514211 | 120514999 | |
| 37955 | 120516570 | 120518731 | 088179 |
| 37956 | 120566530 | 120567672 | 088179 |
| 37957 | 120708463 | 120709067 | 088179 |
| 37958 | 120770225 | 120771805 | 115109 |
| 37959 | 120824098 | 120825209 | 115109 |
| 37960 | 120979900 | 120981841 | 226479 |
| 37961 | 121010224 | 121011623 | 144118 |
| 37962 | 121100598 | 121103220 | |
| 37963 | 121156695 | 121157339 | |
| 37964 | 121199410 | 121200195 | |
| 37965 | 121226913 | 121229195 | |
| 37966 | 121230093 | 121232598 | |
| 37967 | 121238536 | 121239987 | |
| 37968 | 121242629 | 121243312 | |
| 37969 | 121244180 | 121245057 | |
| 37970 | 121261161 | 121262284 | |
| 37971 | 121273885 | 121275605 | |
| 37972 | 121282179 | 121283480 | |
| 37973 | 121297382 | 121299038 | |
| 37974 | 121306074 | 121306765 | |
| 37975 | 121310115 | 121311129 | |
| 37976 | 121383336 | 121383956 | |
| 37977 | 121426702 | 121427211 | |
| 37978 | 121671999 | 121673692 | 074047 |
| 37979 | 121691380 | 121694115 | 074047 |
| 37980 | 121832728 | 121833269 | |
| 37981 | 121968202 | 121969104 | |
| 37982 | 122040332 | 122040979 | 115112 |
| 37983 | 122118153 | 122118539 | 074054 |
| 37984 | 122120548 | 122122356 | 074054 |
| 37985 | 122175846 | 122176676 | 074054 |
| 37986 | 122246468 | 122247348 | 074054 |
| 37987 | 122287285 | 122289053 | 265451, 264229, 074054 |
| 37988 | 122289312 | 122289897 | 265451, 074054 |
| 37989 | 122333259 | 122333891 | 074054 |
| 37990 | 122405379 | 122408724 | 236859, 074054 |
| 37991 | 122456925 | 122457858 | 236859 |
| 37992 | 122493683 | 122494887 | 211460, 155438 |
| 37993 | 122512286 | 122514262 | 211460 |
| 37994 | 122524171 | 122524860 | 211460 |
| 37995 | 122536775 | 122537171 | |
| 37996 | 122567107 | 122567696 | |
| 37997 | 122993756 | 122994459 | |
| 37998 | 123123920 | 123124409 | |
| 37999 | 123190658 | 123191352 | |
| 38000 | 123331569 | 123332247 | |
| 38001 | 123391661 | 123393011 | |
| 38002 | 123408250 | 123412011 | |
| 38003 | 123454706 | 123455986 | |
| 38004 | 123462469 | 123467392 | |
| 38005 | 123467481 | 123468060 | |
| 38006 | 123505652 | 123506895 | |
| 38007 | 125091297 | 125091840 | 155052 |
| 38008 | 125101241 | 125102226 | 155052 |
| 38009 | 125323862 | 125324596 | 155052 |
| 38010 | 125843492 | 125844834 | |
| 38011 | 126808543 | 126809349 | |
| 38012 | 127205919 | 127207022 | |
| 38013 | 127413274 | 127414119 | 136732 |
| 38014 | 127534344 | 127535476 | |
| 38015 | 127728912 | 127730045 | |
| 38016 | 127754157 | 127755216 | |
| 38017 | 127810988 | 127812210 | 136717 |
| 38018 | 127818055 | 127818547 | 136717 |
| 38019 | 127821576 | 127823483 | 136717 |
| 38020 | 127825133 | 127826205 | 136717 |
| 38021 | 127841327 | 127842050 | 136717 |
| 38022 | 127862962 | 127865538 | 136717 |
| 38023 | 127872976 | 127873832 | |
| 38024 | 127886163 | 127886682 | |
| 38025 | 127951086 | 127952740 | 186684 |
| 38026 | 127984031 | 127984822 | |
| 38027 | 127988641 | 127989507 | |
| 38028 | 128050950 | 128052485 | 163161 |
| 38029 | 128083906 | 128084564 | 169967 |
| 38030 | 128104492 | 128105232 | 169967 |
| 38031 | 128115434 | 128116088 | 169967 |
| 38032 | 128143600 | 128146541 | 169967 |
| 38033 | 128155302 | 128156197 | 236682 |
| 38034 | 128164742 | 128167052 | |
| 38035 | 128167482 | 128170235 | |
| 38036 | 128170902 | 128174800 | |
| 38037 | 128175659 | 128178362 | 115718 |
| 38038 | 128195344 | 128196561 | 163166 |
| 38039 | 128211678 | 128213691 | 163166 |
| 38040 | 128266041 | 128266698 | 163166 |
| 38041 | 128282673 | 128285638 | 163166 |
| 38042 | 128421472 | 128423697 | 072163 |
| 38043 | 128431728 | 128433946 | 072163 |
| 38044 | 128457554 | 128460746 | 173349, 136709 |
| 38045 | 128492866 | 128494177 | 136709 |
| 38046 | 128495363 | 128495955 | 136709 |
| 38047 | 128567766 | 128569886 | 136709 |
| 38048 | 128614697 | 128616202 | 144231 |
| 38049 | 128784076 | 128786250 | 136715 |
| 38050 | 128847977 | 128849592 | 136731 |
| 38051 | 128944422 | 128945498 | 136731 |
| 38052 | 128990229 | 128991566 | |
| 38053 | 129063510 | 129064344 | 136720 |
| 38054 | 129066904 | 129067746 | 136720 |
| 38055 | 129079292 | 129080630 | |
| 38056 | 129083163 | 129083723 | |
| 38057 | 129292300 | 129292816 | |
| 38058 | 129350891 | 129352291 | |
| 38059 | 129352398 | 129353227 | |
| 38060 | 129827311 | 129827925 | 238277 |
| 38061 | 129832020 | 129832574 | |
| 38062 | 130634406 | 130635673 | 229536 |
| 38063 | 130938543 | 130940050 | 152082, 136699 |
| 38064 | 131099221 | 131101293 | 136718, 136710 |
| 38065 | 131112551 | 131114518 | 072135 |
| 38066 | 131148718 | 131150112 | |
| 38067 | 131554941 | 131556080 | |
| 38068 | 131774648 | 131775162 | 136002 |
| 38069 | 131834221 | 131835440 | 152102 |
| 38070 | 131849965 | 131852956 | 152102 |
| 38071 | 131861327 | 131864413 | 115762 |
| 38072 | 132249715 | 132251398 | 152117, 173272 |
| 38073 | 133011740 | 133012179 | 163046 |
| 38074 | 133012570 | 133013079 | 163046 |
| 38075 | 133104545 | 133105434 | 272769 |
| 38076 | 133172625 | 133175256 | 183840 |
| 38077 | 133181872 | 133183537 | 183840 |
| 38078 | 133300485 | 133301414 | 183840 |
| 38079 | 133317830 | 133319966 | 183840 |
| 38080 | 133378660 | 133379766 | 183840 |
| 38081 | 133389321 | 133390556 | 183840 |
| 38082 | 133427352 | 133429502 | 176771, 150551 |
| 38083 | 133915485 | 133916535 | 176771 |
| 38084 | 133951418 | 133953166 | 176771 |
| 38085 | 133956126 | 133956825 | 176771 |
| 38086 | 133957402 | 133959695 | 176771 |
| 38087 | 134023027 | 134024533 | 176771, 226953 |
| 38088 | 134104231 | 134104762 | 176771 |
| 38089 | 134178727 | 134179780 | 176771 |
| 38090 | 134252678 | 134253285 | 176771 |
| 38091 | 134260825 | 134261399 | 176771 |
| 38092 | 134262515 | 134263100 | 176771 |
| 38093 | 134323909 | 134326680 | 176771 |
| 38094 | 134333655 | 134335205 | |
| 38095 | 134336277 | 134337426 | |
| 38096 | 134356475 | 134357556 | |
| 38097 | 134424003 | 134425858 | |
| 38098 | 134429977 | 134433088 | |
| 38099 | 134475982 | 134476803 | |
| 38100 | 134523110 | 134523971 | |
| 38101 | 134534544 | 134535248 | |
| 38102 | 134877147 | 134879366 | 152127 |
| 38103 | 134881428 | 134882626 | 152127 |
| 38104 | 134888956 | 134889755 | 152127 |
| 38105 | 134897127 | 134898466 | 152127 |
| 38106 | 134945948 | 134947220 | 152127 |
| 38107 | 135159848 | 135160385 | 152127 |
| 38108 | 135188945 | 135191680 | 152127 |
| 38109 | 135208084 | 135209072 | 152127 |
| 38110 | 135441009 | 135441890 | 152128 |
| 38111 | 135580894 | 135582237 | 224043 |
| 38112 | 135593776 | 135598556 | 153086, 224043 |
| 38113 | 135618816 | 135621706 | 224043, 153086 |
| 38114 | 135622043 | 135625315 | 224043, 153086 |
| 38115 | 135627622 | 135628991 | 224043, 153086 |
| 38116 | 135674583 | 135677659 | 082258, 224043 |
| 38117 | 135690300 | 135692695 | 082258 |
| 38118 | 135809305 | 135811072 | 115839 |
| 38119 | 135849075 | 135849774 | 115839 |
| 38120 | 136287556 | 136290332 | 048991, 121988 |
| 38121 | 136392312 | 136393414 | 048991 |
| 38122 | 136498666 | 136501038 | 144224 |
| 38123 | 136532806 | 136533362 | 144224 |
| 38124 | 136633223 | 136634197 | 076003 |
| 38125 | 136705578 | 136707528 | 115866 |
| 38126 | 136741538 | 136744014 | 231890, 115866 |
| 38127 | 137667294 | 137667811 | 144229 |
| 38128 | 138686189 | 138687424 | |
| 38129 | 138698904 | 138701320 | |
| 38130 | 138709370 | 138711292 | |
| 38131 | 138720823 | 138724825 | 150540 |
| 38132 | 138835768 | 138836117 | |
| 38133 | 138838735 | 138840206 | |
| 38134 | 138867824 | 138868999 | |
| 38135 | 139024245 | 139026199 | |
| 38136 | 139032065 | 139035020 | |
| 38137 | 139113627 | 139114168 | |
| 38138 | 139152739 | 139154812 | |
| 38139 | 139177871 | 139180418 | 228043 |
| 38140 | 139200881 | 139201323 | 228043 |
| 38141 | 139258293 | 139260633 | 144228, 228043 |
| 38142 | 139272395 | 139273352 | 144228 |
| 38143 | 139273630 | 139274813 | 144228 |
| 38144 | 139339389 | 139340367 | |
| 38145 | 142087208 | 142089092 | 168702 |
| 38146 | 142468074 | 142468637 | 168702 |
| 38147 | 143172867 | 143173965 | |
| 38148 | 143229829 | 143230439 | |
| 38149 | 143344125 | 143345065 | |
| 38150 | 143503108 | 143503834 | |
| 38151 | 143629944 | 143639059 | 115919 |
| 38152 | 143708866 | 143709628 | 115919 |
| 38153 | 144955413 | 144956347 | 121964 |
| 38154 | 144987758 | 144988490 | 121964 |
| 38155 | 145077633 | 145078503 | 121964 |
| 38156 | 145089031 | 145090949 | 121964 |
| 38157 | 145115265 | 145116924 | |
| 38158 | 145128549 | 145129360 | |
| 38159 | 145161508 | 145161986 | 169554 |
| 38160 | 146512535 | 146513464 | |
| 38161 | 146518083 | 146519375 | |
| 38162 | 146557633 | 146558413 | |
| 38163 | 146971338 | 146973022 | |
| 38164 | 147020015 | 147020718 | |
| 38165 | 147844307 | 147844784 | |
| 38166 | 148139643 | 148140614 | |
| 38167 | 148173065 | 148174664 | |
| 38168 | 148182384 | 148183711 | |
| 38169 | 148187477 | 148188540 | |
| 38170 | 148191213 | 148191945 | |
| 38171 | 148257026 | 148258009 | |
| 38172 | 148334886 | 148335312 | |
| 38173 | 148601066 | 148603572 | 121989 |
| 38174 | 148684539 | 148685248 | 121989 |
| 38175 | 148777402 | 148779699 | 204406, 115947 |
| 38176 | 148887960 | 148888559 | 204406 |
| 38177 | 149013371 | 149014304 | 204406 |
| 38178 | 149017007 | 149017654 | 204406 |
| 38179 | 149030461 | 149031327 | 204406 |
| 38180 | 149266620 | 149267575 | 204406 |
| 38181 | 149401654 | 149403682 | 135999 |
| 38182 | 150186150 | 150187523 | 187123 |
| 38183 | 150276381 | 150277497 | 187123 |
| 38184 | 150278803 | 150279827 | 187123 |
| 38185 | 150418701 | 150419512 | |
| 38186 | 150443060 | 150445012 | 231969, 168288 |
| 38187 | 150507708 | 150508301 | 231969 |
| 38188 | 150510144 | 150511053 | 231969 |
| 38189 | 150733813 | 150735110 | |
| 38190 | 150741712 | 150742498 | |
| 38191 | 150842415 | 150843112 | |
| 38192 | 150845061 | 150847217 | |
| 38193 | 150850837 | 150851383 | |
| 38194 | 150857754 | 150858845 | |
| 38195 | 150954416 | 150955255 | |
| 38196 | 150981310 | 150983712 | |
| 38197 | 150989842 | 150992652 | |
| 38198 | 151021155 | 151027020 | |
| 38199 | 151035101 | 151035718 | 230645 |
| 38200 | 151044649 | 151046150 | 230645 |
| 38201 | 151071072 | 151071663 | 230645 |
| 38202 | 151111951 | 151112778 | 231420, 230645 |
| 38203 | 151139300 | 151140028 | 230645 |
| 38204 | 151152919 | 151154125 | 230645 |
| 38205 | 151175138 | 151177610 | |
| 38206 | 151177638 | 151178408 | |
| 38207 | 151214044 | 151215009 | |
| 38208 | 151227578 | 151228431 | |
| 38209 | 151232987 | 151233625 | |
| 38210 | 151267245 | 151268032 | |
| 38211 | 151328697 | 151329371 | 115963 |
| 38212 | 151332730 | 151334479 | 115963 |
| 38213 | 151335659 | 151337304 | 115963 |
| 38214 | 151340747 | 151344657 | 115963 |
| 38215 | 151347909 | 151349593 | 115963 |
| 38216 | 151385115 | 151386558 | 115963 |
| 38217 | 151408778 | 151410347 | 236049 |
| 38218 | 151412167 | 151412971 | 236049 |
| 38219 | 151443149 | 151445701 | |
| 38220 | 151446100 | 151447565 | |
| 38221 | 151454818 | 151455365 | 232359 |
| 38222 | 151457655 | 151459034 | 232359 |
| 38223 | 151469273 | 151470223 | 232359, 224048 |
| 38224 | 151474134 | 151475776 | 232359, 224048 |
| 38225 | 151475888 | 151479324 | 232359, 224048 |
| 38226 | 151486911 | 151488205 | 224048 |
| 38227 | 151503469 | 151504218 | |
| 38228 | 151542872 | 151543493 | |
| 38229 | 151767740 | 151768968 | |
| 38230 | 151905961 | 151907992 | |
| 38231 | 152116933 | 152118812 | 184898 |
| 38232 | 152120638 | 152121228 | |
| 38233 | 152145541 | 152147475 | 123609 |
| 38234 | 152148593 | 152149298 | |
| 38235 | 152266069 | 152267493 | 080345 |
| 38236 | 152486783 | 152487355 | 183091 |
| 38237 | 152494727 | 152495587 | 183091 |
| 38238 | 152683982 | 152685560 | 162980 |
| 38239 | 153028090 | 153028738 | 115145 |
| 38240 | 153031333 | 153033350 | 115145 |
| 38241 | 153191221 | 153192862 | 157827 |
| 38242 | 153261634 | 153262194 | 157827 |
| 38243 | 153278021 | 153279087 | 157827 |
| 38244 | 153288552 | 153289210 | 157827 |
| 38245 | 153302090 | 153302976 | 157827 |
| 38246 | 153550714 | 153551522 | 196504 |
| 38247 | 153572827 | 153575958 | 177917, 196504 |
| 38248 | 155306254 | 155307210 | 224675, 144278 |
| 38249 | 155457310 | 155458118 | |
| 38250 | 155553892 | 155554792 | 235949 |
| 38251 | 155555149 | 155556515 | 162989 |
| 38252 | 156832175 | 156832757 | |
| 38253 | 157176272 | 157177313 | |
| 38254 | 157188366 | 157189050 | 153234 |
| 38255 | 157189065 | 157192597 | 153234 |
| 38256 | 157197545 | 157199910 | 153234 |
| 38257 | 157255449 | 157258141 | |
| 38258 | 157262000 | 157262921 | |
| 38259 | 157291473 | 157293520 | 115159 |
| 38260 | 157515791 | 157516824 | |
| 38261 | 157659329 | 157660561 | |
| 38262 | 158039127 | 158040208 | |
| 38263 | 158102413 | 158104145 | |
| 38264 | 158186070 | 158186897 | |
| 38265 | 158223973 | 158224753 | |
| 38266 | 158336598 | 158337520 | 115165 |
| 38267 | 158345169 | 158345898 | 115165 |
| 38268 | 158484857 | 158486157 | 123612 |
| 38269 | 158589040 | 158589535 | 234584 |
| 38270 | 158598222 | 158599198 | 115170 |
| 38271 | 158602724 | 158603587 | 115170 |
| 38272 | 158603975 | 158604941 | 115170 |
| 38273 | 158620698 | 158621567 | 115170 |
| 38274 | 158640569 | 158641849 | 115170 |
| 38275 | 158678036 | 158679343 | 115170 |
| 38276 | 158686607 | 158689336 | 115170 |
| 38277 | 158692942 | 158696161 | 115170 |
| 38278 | 158696590 | 158698417 | 115170 |
| 38279 | 158698554 | 158699493 | 115170 |
| 38280 | 158730750 | 158733985 | 007001, 115170 |
| 38281 | 158737232 | 158737933 | 007001 |
| 38282 | 158788031 | 158788551 | 007001 |
| 38283 | 158794364 | 158795680 | 007001 |
| 38284 | 158796425 | 158797051 | 007001 |
| 38285 | 158899437 | 158900160 | 007001 |
| 38286 | 158913203 | 158913921 | 007001 |
| 38287 | 158955452 | 158958366 | 007001 |
| 38288 | 158984634 | 158985745 | 007001, 237327 |
| 38289 | 158986866 | 158989152 | 007001, 237327 |
| 38290 | 159047542 | 159048642 | 227480, 153237 |
| 38291 | 159250777 | 159252655 | 153237 |
| 38292 | 159312826 | 159315434 | 144283, 153237 |
| 38293 | 159315987 | 159316597 | 144283 |
| 38294 | 159353939 | 159354402 | 144283 |
| 38295 | 159395625 | 159396167 | 144283 |
| 38296 | 159475264 | 159476495 | 144283 |
| 38297 | 159552267 | 159552809 | 204380, 228586 |
| 38298 | 159684957 | 159685606 | 163331 |
| 38299 | 159810268 | 159811321 | |
| 38300 | 159824266 | 159826747 | 115183 |
| 38301 | 159851361 | 159852177 | 115183 |
| 38302 | 159954909 | 159956352 | 115183 |
| 38303 | 160059036 | 160059603 | 115183 |
| 38304 | 160142399 | 160144265 | 196151 |
| 38305 | 160362740 | 160363750 | 123636 |
| 38306 | 160470589 | 160474019 | 224152, 123636 |
| 38307 | 160474152 | 160474993 | |
| 38308 | 160567944 | 160570615 | 136536, 226266 |
| 38309 | 160653624 | 160655512 | 241399, 248672, 054219 |
| 38310 | 161060986 | 161067886 | 115221 |
| 38311 | 161070442 | 161071810 | 115221 |
| 38312 | 161077514 | 161090484 | 115221 |
| 38313 | 161097731 | 161099398 | 115221 |
| 38314 | 161107992 | 161111148 | 115221 |
| 38315 | 161124357 | 161128073 | 115221 |
| 38316 | 161262739 | 161264442 | 263948, 153250 |
| 38317 | 161289016 | 161289654 | 153250 |
| 38318 | 161348559 | 161350703 | 153250 |
| 38319 | 161826446 | 161827099 | |
| 38320 | 161886880 | 161887441 | |
| 38321 | 161979163 | 161980676 | 224467 |
| 38322 | 162015804 | 162017936 | 224467, 136560 |
| 38323 | 162100629 | 162102397 | 227403, 235724 |
| 38324 | 162107208 | 162107990 | 235724 |
| 38325 | 162113520 | 162114755 | |
| 38326 | 162164151 | 162166368 | 115233 |
| 38327 | 162878650 | 162879505 | 197635 |
| 38328 | 162883983 | 162884813 | 197635 |
| 38329 | 162885685 | 162886533 | 197635 |
| 38330 | 162906879 | 162907559 | 197635 |
| 38331 | 162929499 | 162932761 | 230918, 197635 |
| 38332 | 162941017 | 162943844 | |
| 38333 | 162944619 | 162950101 | 224674, 233397 |
| 38334 | 162956909 | 162957571 | |
| 38335 | 162974955 | 162975638 | |
| 38336 | 163041045 | 163041982 | 078098 |
| 38337 | 163174067 | 163176188 | 115271, 115267 |
| 38338 | 163200197 | 163201355 | 115271 |
| 38339 | 163430868 | 163432531 | 184611 |
| 38340 | 163436415 | 163436884 | 184611 |
| 38341 | 163438686 | 163439770 | 184611 |
| 38342 | 163442649 | 163443180 | 184611 |
| 38343 | 163464077 | 163465383 | 184611 |
| 38344 | 163544762 | 163545412 | 184611 |
| 38345 | 163566660 | 163567649 | 184611 |
| 38346 | 164066909 | 164067667 | |
| 38347 | 164521637 | 164525249 | 182263 |
| 38348 | 164564584 | 164565521 | 182263 |
| 38349 | 164591226 | 164593782 | 182263 |
| 38350 | 164630646 | 164631938 | 237844 |
| 38351 | 164636464 | 164637017 | 237844 |
| 38352 | 164651087 | 164651783 | 237844 |
| 38353 | 165370857 | 165371757 | 115290 |
| 38354 | 165423702 | 165424537 | 115290 |
| 38355 | 165477233 | 165479803 | 115290 |
| 38356 | 165511284 | 165512489 | 082438 |
| 38357 | 165516959 | 165518341 | 082438 |
| 38358 | 165550634 | 165551498 | 082438 |
| 38359 | 165567071 | 165569020 | 082438 |
| 38360 | 165596376 | 165597107 | 082438 |
| 38361 | 165622601 | 165623109 | 082438 |
| 38362 | 165638718 | 165639212 | 082438 |
| 38363 | 165667243 | 165667726 | 082438 |
| 38364 | 165695514 | 165699241 | 233255, 082438 |
| 38365 | 165732149 | 165732824 | |
| 38366 | 165734997 | 165735795 | |
| 38367 | 165743771 | 165744593 | |
| 38368 | 165753048 | 165754272 | 169507 |
| 38369 | 165776238 | 165778152 | 169507 |
| 38370 | 165793912 | 165795043 | 169507 |
| 38371 | 165864887 | 165867096 | 236283 |
| 38372 | 165912813 | 165913537 | 236283 |
| 38373 | 166430037 | 166431546 | 178662 |
| 38374 | 166809294 | 166810995 | 123607 |
| 38375 | 167086094 | 167087801 | 169432, 236107 |
| 38376 | 167227702 | 167233505 | 169432 |
| 38377 | 167324485 | 167325295 | 136546 |
| 38378 | 168604733 | 168606557 | |
| 38379 | 168617955 | 168618632 | |
| 38380 | 168621187 | 168621958 | |
| 38381 | 168671436 | 168672484 | 235335 |
| 38382 | 168683400 | 168684699 | 172318, 235335 |
| 38383 | 169066845 | 169069746 | 198648 |
| 38384 | 169206616 | 169207334 | |
| 38385 | 169261125 | 169261863 | |
| 38386 | 169311939 | 169313969 | 172292 |
| 38387 | 169334536 | 169335339 | 172292 |
| 38388 | 169415361 | 169416226 | 172292 |
| 38389 | 169637021 | 169637788 | 227617 |
| 38390 | 169706054 | 169706472 | 163072, 152253 |
| 38391 | 169746760 | 169747330 | 152253 |
| 38392 | 169769846 | 169770534 | 152253 |
| 38393 | 169817992 | 169818678 | 073734 |
| 38394 | 169848418 | 169850129 | 073734 |
| 38395 | 169867378 | 169872010 | 073734 |
| 38396 | 169884564 | 169888811 | 073734 |
| 38397 | 169925073 | 169926594 | 073737 |
| 38398 | 169928153 | 169931909 | 073737 |
| 38399 | 170114528 | 170115046 | 081479 |
| 38400 | 170220462 | 170221418 | |
| 38401 | 170242860 | 170243768 | |
| 38402 | 170335635 | 170336684 | 163093, 251569 |
| 38403 | 170429512 | 170430707 | 138399 |
| 38404 | 170440148 | 170442866 | 138398 |
| 38405 | 170550462 | 170551763 | 144362, 213160, 154479 |
| 38406 | 170571714 | 170572560 | 213160 |
| 38407 | 170584929 | 170585399 | 213160 |
| 38408 | 170589617 | 170591496 | 213160 |
| 38409 | 170618056 | 170618549 | 213160 |
| 38410 | 170624649 | 170625749 | 231550, 213160 |
| 38411 | 170655073 | 170656457 | 138385 |
| 38412 | 170680612 | 170681796 | 138382 |
| 38413 | 170683713 | 170685016 | 144357 |
| 38414 | 170708480 | 170709174 | 144357 |
| 38415 | 170886394 | 170886987 | 144357 |
| 38416 | 171281752 | 171282423 | 071909 |
| 38417 | 171311895 | 171313125 | 071909 |
| 38418 | 171321611 | 171322534 | 071909 |
| 38419 | 171341556 | 171342394 | 071909 |
| 38420 | 171381469 | 171382674 | 071909 |
| 38421 | 171386603 | 171387560 | 071909 |
| 38422 | 171410643 | 171411447 | 071909 |
| 38423 | 171570309 | 171572466 | 204335, 222033 |
| 38424 | 171600255 | 171601392 | 234350 |
| 38425 | 171626670 | 171628143 | 239467, 234350 |
| 38426 | 171729512 | 171730342 | |
| 38427 | 171751497 | 171752431 | |
| 38428 | 171784652 | 171787254 | 115806 |
| 38429 | 171829185 | 171831140 | |
| 38430 | 172015486 | 172018470 | 198586 |
| 38431 | 172022686 | 172023535 | 198586 |
| 38432 | 172128224 | 172129070 | |
| 38433 | 172239597 | 172240135 | 123600 |
| 38434 | 172289331 | 172291669 | 115827, 123600 |
| 38435 | 172379938 | 172380344 | 071967 |
| 38436 | 172543678 | 172545186 | 077380 |
| 38437 | 172585625 | 172586614 | 077380 |
| 38438 | 172694081 | 172694804 | 115840 |
| 38439 | 172750392 | 172751243 | 115840 |
| 38440 | 172778489 | 172779786 | 128708, 115840 |
| 38441 | 172864229 | 172865928 | 172878, 115840 |
| 38442 | 172872967 | 172874073 | 172878 |
| 38443 | 172916804 | 172917543 | 172878 |
| 38444 | 173117806 | 173118944 | |
| 38445 | 173203752 | 173206258 | |
| 38446 | 173213088 | 173214131 | |
| 38447 | 173215649 | 173216880 | |
| 38448 | 173238957 | 173240396 | |
| 38449 | 173244878 | 173246190 | |
| 38450 | 173291674 | 173294982 | 091409, 226963 |
| 38451 | 173301643 | 173304417 | 091409 |
| 38452 | 173306762 | 173307771 | 091409 |
| 38453 | 173329812 | 173330897 | 232788, 091409 |
| 38454 | 173419840 | 173421998 | 152256, 225205 |
| 38455 | 173536241 | 173539087 | 230104 |
| 38456 | 173590437 | 173591908 | 228016 |
| 38457 | 173599997 | 173601295 | 091428, 228016 |
| 38458 | 173721042 | 173721747 | 091428 |
| 38459 | 173786120 | 173786913 | 091428 |
| 38460 | 173792368 | 173793205 | 091428 |
| 38461 | 173831377 | 173834144 | 091428 |
| 38462 | 173849594 | 173851529 | 091428 |
| 38463 | 173860456 | 173861461 | 091428 |
| 38464 | 173907659 | 173909068 | 091428 |
| 38465 | 173912467 | 173916269 | 091428 |
| 38466 | 173916313 | 173919943 | 091428 |
| 38467 | 173922702 | 173924203 | |
| 38468 | 173924330 | 173925377 | |
| 38469 | 173939242 | 173944244 | 091436 |
| 38470 | 173951157 | 173951681 | 091436 |
| 38471 | 173965650 | 173967354 | 091436 |
| 38472 | 174024416 | 174026683 | 091436 |
| 38473 | 174095738 | 174096698 | 091436, 238133 |
| 38474 | 174292446 | 174293699 | |
| 38475 | 174409308 | 174410466 | |
| 38476 | 174715671 | 174716687 | |
| 38477 | 174719164 | 174720178 | |
| 38478 | 174827333 | 174831990 | 172845 |
| 38479 | 174850883 | 174852162 | |
| 38480 | 174855658 | 174857563 | |
| 38481 | 174868263 | 174870222 | |
| 38482 | 174904020 | 174905194 | |
| 38483 | 175039045 | 175040048 | 138430 |
| 38484 | 175112451 | 175114412 | 138430 |
| 38485 | 175259515 | 175261338 | 144306, 138433 |
| 38486 | 175307302 | 175308001 | 163328 |
| 38487 | 175350792 | 175352978 | 226853, 163328 |
| 38488 | 175383137 | 175383539 | |
| 38489 | 175425826 | 175426636 | 115935, 236449 |
| 38490 | 175449843 | 175450336 | 237798, 115935, 236449 |
| 38491 | 175481618 | 175482253 | 115935, 236449 |
| 38492 | 175628015 | 175628563 | 138435, 236449 |
| 38493 | 175832365 | 175832971 | 128656 |
| 38494 | 175895096 | 175896162 | |
| 38495 | 176031828 | 176033295 | 229750, 215973, 115966 |
| 38496 | 176045259 | 176047171 | 154518 |
| 38497 | 176464607 | 176465597 | |
| 38498 | 176470350 | 176471897 | |
| 38499 | 176495468 | 176496174 | |
| 38500 | 176650778 | 176651542 | |
| 38501 | 176654800 | 176655447 | |
| 38502 | 176865848 | 176867856 | 144320 |
| 38503 | 176961237 | 176962764 | |
| 38504 | 177133726 | 177135168 | 128654 |
| 38505 | 177248573 | 177249416 | |
| 38506 | 177447573 | 177448138 | |
| 38507 | 177453943 | 177454595 | |
| 38508 | 177678893 | 177679892 | 230552 |
| 38509 | 177943102 | 177943584 | 236501 |
| 38510 | 178029112 | 178031816 | 236501, 229337 |
| 38511 | 178032384 | 178038088 | 229337 |
| 38512 | 178051768 | 178053952 | 229337 |
| 38513 | 178075544 | 178078728 | 263721, 170144, 229337 |
| 38514 | 178106547 | 178108373 | 116044 |
| 38515 | 178115867 | 178118111 | 116044 |
| 38516 | 178127633 | 178130984 | 222043, 116044 |
| 38517 | 178132818 | 178133580 | 116044 |
| 38518 | 178137924 | 178138794 | 116044 |
| 38519 | 178146043 | 178147320 | 116044 |
| 38520 | 178181149 | 178182983 | 213963, 116044 |
| 38521 | 178187211 | 178188257 | 213963, 116044 |
| 38522 | 178189705 | 178190921 | 213963, 116044 |
| 38523 | 178202247 | 178204074 | 213963, 116044 |
| 38524 | 178256680 | 178258725 | 018510, 213963, 116044 |
| 38525 | 178261368 | 178261764 | 018510 |
| 38526 | 178262330 | 178263496 | 018510 |
| 38527 | 178265226 | 178265646 | 018510 |
| 38528 | 178417266 | 178418444 | 196659 |
| 38529 | 178483186 | 178484328 | 197557, 237655 |
| 38530 | 178556777 | 178559069 | 128655 |
| 38531 | 178588946 | 178589898 | 128655 |
| 38532 | 178648663 | 178650152 | 128655 |
| 38533 | 178720172 | 178720702 | 128655 |
| 38534 | 178738300 | 178739492 | 128655 |
| 38535 | 178753260 | 178753716 | 128655 |
| 38536 | 178853395 | 178854743 | 236664, 128655 |
| 38537 | 178935424 | 178936183 | 128655 |
| 38538 | 178936756 | 178938563 | 128655 |
| 38539 | 178976794 | 178978361 | 155636 |
| 38540 | 179058106 | 179060371 | 079156 |
| 38541 | 179106923 | 179108104 | 079156 |
| 38542 | 179136741 | 179137308 | 079156 |
| 38543 | 179163451 | 179163879 | 079156 |
| 38544 | 179314774 | 179316900 | 204311, 180228 |
| 38545 | 179333976 | 179334844 | 079150 |
| 38546 | 179342484 | 179344151 | 079150 |
| 38547 | 179344707 | 179345376 | 116095 |
| 38548 | 179387540 | 179388578 | 237298 |
| 38549 | 179971189 | 179972099 | 187231 |
| 38550 | 179972173 | 179973281 | 187231 |
| 38551 | 180103683 | 180104322 | 187231 |
| 38552 | 180127681 | 180128142 | 187231 |
| 38553 | 180128150 | 180130284 | 187231 |
| 38554 | 180136745 | 180137983 | |
| 38555 | 180324666 | 180326537 | 144331 |
| 38556 | 180386599 | 180387051 | 144331 |
| 38557 | 180422776 | 180423759 | 144331 |
| 38558 | 180438278 | 180439510 | 144331 |
| 38559 | 180444291 | 180445700 | 144331 |
| 38560 | 180701540 | 180703374 | 144331 |
| 38561 | 180724980 | 180726667 | 221240, 144331 |
| 38562 | 180781950 | 180782482 | |
| 38563 | 180871032 | 180872433 | 163510 |
| 38564 | 181383265 | 181387357 | |
| 38565 | 181509427 | 181509907 | 225258 |
| 38566 | 181519966 | 181522914 | 225258 |
| 38567 | 181524418 | 181525451 | 225258 |
| 38568 | 181540614 | 181543771 | 225258 |
| 38569 | 181587269 | 181587682 | |
| 38570 | 181606779 | 181608590 | |
| 38571 | 181612161 | 181613035 | |
| 38572 | 181625901 | 181626404 | |
| 38573 | 181843960 | 181846994 | 236153, 170035 |
| 38574 | 182655228 | 182656548 | |
| 38575 | 182755873 | 182758395 | 138434, 260742 |
| 38576 | 182774526 | 182775356 | 138434 |
| 38577 | 182808484 | 182809564 | |
| 38578 | 182818584 | 182820770 | 150722 |
| 38579 | 182903333 | 182903943 | 150722 |
| 38580 | 182931328 | 182932508 | 150722 |
| 38581 | 182941757 | 182942967 | 150722 |
| 38582 | 183579741 | 183581796 | 077232 |
| 38583 | 183755512 | 183756712 | |
| 38584 | 183776450 | 183777322 | 061676 |
| 38585 | 183779777 | 183781534 | 061676 |
| 38586 | 183901173 | 183903891 | 061676 |
| 38587 | 183942709 | 183944342 | 162999 |
| 38588 | 183988309 | 183990322 | 163002 |
| 38589 | 185126097 | 185127316 | |
| 38590 | 186447444 | 186449087 | |
| 38591 | 186686007 | 186686686 | 188738 |
| 38592 | 187243541 | 187244234 | |
| 38593 | 187349936 | 187352276 | 065548 |
| 38594 | 187398142 | 187399021 | |
| 38595 | 187408014 | 187411347 | |
| 38596 | 187434867 | 187436044 | |
| 38597 | 187439204 | 187441924 | |
| 38598 | 187453477 | 187459004 | 138448 |
| 38599 | 188296444 | 188297069 | 224063, 064989 |
| 38600 | 188364857 | 188367455 | 224063, 003436 |
| 38601 | 188391911 | 188392399 | 224063, 003436 |
| 38602 | 188393852 | 188396460 | 224063, 003436 |
| 38603 | 188404521 | 188420213 | 224063, 003436 |
| 38604 | 188712005 | 188712747 | 231689 |
| 38605 | 188847187 | 188847780 | 231689 |
| 38606 | 188850671 | 188851065 | 231689 |
| 38607 | 189299649 | 189300565 | 144366 |
| 38608 | 189914882 | 189915643 | 204262 |
| 38609 | 189969565 | 189970359 | 204262 |
| 38610 | 189974391 | 189975297 | 204262 |
| 38611 | 190014685 | 190015175 | 204262 |
| 38612 | 190028476 | 190029848 | 204262 |
| 38613 | 190042421 | 190043783 | 204262 |
| 38614 | 190234275 | 190234785 | |
| 38615 | 190305109 | 190307191 | 115368 |
| 38616 | 190331266 | 190332837 | 115368 |
| 38617 | 190386244 | 190388368 | |
| 38618 | 190391929 | 190393463 | |
| 38619 | 190397754 | 190401177 | |
| 38620 | 190406327 | 190407635 | |
| 38621 | 190413110 | 190414792 | |
| 38622 | 190416135 | 190418713 | |
| 38623 | 190419445 | 190421094 | |
| 38624 | 190435215 | 190435825 | 138449 |
| 38625 | 190439388 | 190440625 | 138449 |
| 38626 | 190443969 | 190446643 | 138449 |
| 38627 | 190476933 | 190478928 | |
| 38628 | 190525552 | 190527182 | 138381 |
| 38629 | 190538844 | 190540327 | 151687 |
| 38630 | 190626719 | 190628601 | 273240, 253559, 128694 |
| 38631 | 190647680 | 190650141 | 064933, 128699 |
| 38632 | 190934671 | 190935533 | 187699 |
| 38633 | 191044577 | 191046166 | 187699 |
| 38634 | 191131896 | 191132912 | 198130 |
| 38635 | 191143251 | 191145356 | 198130 |
| 38636 | 191183395 | 191185292 | 198130 |
| 38637 | 191197969 | 191200277 | 198130 |
| 38638 | 191207641 | 191209593 | 151689, 198130 |
| 38639 | 191217345 | 191218193 | 151689 |
| 38640 | 191272792 | 191273671 | 151690 |
| 38641 | 191398189 | 191400288 | 233654, 189362 |
| 38642 | 191501050 | 191501587 | |
| 38643 | 191512490 | 191515865 | 138386 |
| 38644 | 191580565 | 191581626 | |
| 38645 | 191583325 | 191584064 | |
| 38646 | 191673793 | 191674966 | |
| 38647 | 191677518 | 191678032 | |
| 38648 | 191744913 | 191747259 | 235852, 115419 |
| 38649 | 191877775 | 191879727 | 115415 |
| 38650 | 191884797 | 191886879 | 231858, 115415 |
| 38651 | 191906373 | 191906866 | 138378 |
| 38652 | 191914477 | 191915321 | 138378 |
| 38653 | 192015102 | 192015907 | 138378 |
| 38654 | 192029748 | 192038300 | |
| 38655 | 192041083 | 192046599 | |
| 38656 | 192047389 | 192050109 | |
| 38657 | 192051649 | 192052822 | |
| 38658 | 192066468 | 192068422 | |
| 38659 | 192077901 | 192078498 | |
| 38660 | 192080226 | 192080821 | |
| 38661 | 192081716 | 192085480 | |
| 38662 | 192109046 | 192113079 | 128641, 227542 |
| 38663 | 192127283 | 192128324 | 128641 |
| 38664 | 192129594 | 192131470 | 128641 |
| 38665 | 192175713 | 192177452 | 128641 |
| 38666 | 192223039 | 192223898 | 128641 |
| 38667 | 192241067 | 192243019 | 128641 |
| 38668 | 192247471 | 192248092 | 128641 |
| 38669 | 192261388 | 192261943 | 128641 |
| 38670 | 192282540 | 192283414 | 128641 |
| 38671 | 192435733 | 192436583 | |
| 38672 | 192446892 | 192447610 | |
| 38673 | 192458175 | 192459273 | |
| 38674 | 192500510 | 192504100 | |
| 38675 | 192520916 | 192522027 | |
| 38676 | 192531119 | 192532196 | |
| 38677 | 192541345 | 192546194 | 173559 |
| 38678 | 192553419 | 192556621 | 173559 |
| 38679 | 192623491 | 192624073 | |
| 38680 | 192731074 | 192732843 | 233766 |
| 38681 | 193967609 | 193968239 | |
| 38682 | 194292527 | 194294117 | |
| 38683 | 194331828 | 194332969 | |
| 38684 | 194385182 | 194386752 | |
| 38685 | 196408921 | 196409947 | |
| 38686 | 196440543 | 196441334 | 196950 |
| 38687 | 196520708 | 196523627 | 196950 |
| 38688 | 197035351 | 197036627 | 081320 |
| 38689 | 197456832 | 197458475 | 138411 |
| 38690 | 197503815 | 197504756 | 144395 |
| 38691 | 197583294 | 197584678 | 144395 |
| 38692 | 197594003 | 197594759 | 144395 |
| 38693 | 197613348 | 197613975 | 144395 |
| 38694 | 197663587 | 197665034 | 119041 |
| 38695 | 197790884 | 197792310 | 197121 |
| 38696 | 197971636 | 197974461 | 065413 |
| 38697 | 198022451 | 198026107 | 065413 |
| 38698 | 198043981 | 198046039 | 065413 |
| 38699 | 198046921 | 198049139 | 065413 |
| 38700 | 198049246 | 198050825 | 065413 |
| 38701 | 198151673 | 198152438 | 065413, 236977 |
| 38702 | 198171138 | 198171721 | 065413 |
| 38703 | 198174365 | 198176370 | 225979, 065413 |
| 38704 | 198298744 | 198301262 | 115524 |
| 38705 | 198317204 | 198319706 | 115520 |
| 38706 | 198363319 | 198366320 | 270757, 115541, 144381 |
| 38707 | 198379674 | 198381643 | 115540, 144381, 270757 |
| 38708 | 198436110 | 198437454 | 162944 |
| 38709 | 198440829 | 198442031 | 162944 |
| 38710 | 198477949 | 198478763 | 162944 |
| 38711 | 198569301 | 198571371 | 247626, 222017 |
| 38712 | 198668971 | 198670073 | 115896 |
| 38713 | 199173220 | 199174971 | 115896, 236653 |
| 38714 | 199480078 | 199481232 | 225421 |
| 38715 | 200320201 | 200321181 | 119042 |
| 38716 | 200321754 | 200326199 | 225953, 119042 |
| 38717 | 200389541 | 200390319 | |
| 38718 | 200603228 | 200604077 | |
| 38719 | 200714829 | 200716809 | 226124 |
| 38720 | 200775610 | 200776948 | 178074, 232732 |
| 38721 | 200819173 | 200821406 | 162972, 162971, 178074 |
| 38722 | 200836716 | 200837414 | 162972 |
| 38723 | 200841863 | 200845403 | 162972 |
| 38724 | 200914693 | 200916784 | |
| 38725 | 200941209 | 200943073 | |
| 38726 | 200968751 | 200969280 | |
| 38727 | 200992496 | 200993671 | |
| 38728 | 200993794 | 200997963 | |
| 38729 | 201020853 | 201021685 | |
| 38730 | 201023990 | 201024563 | |
| 38731 | 201081915 | 201082517 | |
| 38732 | 201083686 | 201086745 | |
| 38733 | 201123548 | 201124780 | |
| 38734 | 201136932 | 201137639 | |
| 38735 | 201153367 | 201154038 | |
| 38736 | 201165331 | 201168858 | |
| 38737 | 201169803 | 201176234 | 196141 |
| 38738 | 201183562 | 201186286 | 196141 |
| 38739 | 201186567 | 201187509 | 196141 |
| 38740 | 201217288 | 201217977 | 196141 |
| 38741 | 201231779 | 201232663 | 196141 |
| 38742 | 201238088 | 201239320 | 196141 |
| 38743 | 201240729 | 201243611 | 196141 |
| 38744 | 201269248 | 201272180 | 196141 |
| 38745 | 201272285 | 201274009 | 196141 |
| 38746 | 201320046 | 201320961 | 196141 |
| 38747 | 201326592 | 201327355 | 196141 |
| 38748 | 201337497 | 201338368 | 196141 |
| 38749 | 201343859 | 201345694 | 196141 |
| 38750 | 201346032 | 201346533 | 196141 |
| 38751 | 201373762 | 201375483 | 163535, 155729 |
| 38752 | 201390542 | 201391261 | 163535 |
| 38753 | 201450035 | 201453812 | 138356 |
| 38754 | 201457069 | 201457771 | 138356 |
| 38755 | 201467431 | 201470267 | 138356 |
| 38756 | 201472942 | 201475653 | 138356 |
| 38757 | 201477757 | 201484559 | 138356 |
| 38758 | 201487849 | 201491331 | 138356 |
| 38759 | 201513386 | 201514134 | 138356 |
| 38760 | 201542506 | 201543524 | 244301 |
| 38761 | 201567252 | 201568880 | 244301, 243478 |
| 38762 | 201577494 | 201579047 | 237166, 244301, 243478 |
| 38763 | 201611881 | 201615119 | 243478 |
| 38764 | 201619134 | 201622662 | 243478 |
| 38765 | 201629629 | 201631622 | 243478 |
| 38766 | 201640954 | 201644284 | 243478 |
| 38767 | 201651014 | 201652141 | 243478, 230408 |
| 38768 | 201661884 | 201662592 | 230408 |
| 38769 | 201674903 | 201678121 | 082153, 230408 |
| 38770 | 201727284 | 201730409 | 252759, 013441 |
| 38771 | 201731491 | 201732535 | |
| 38772 | 201752830 | 201755091 | 196290, 240344 |
| 38773 | 201808968 | 201810482 | 115942 |
| 38774 | 201827339 | 201828927 | 183308, 115942 |
| 38775 | 201923389 | 201924420 | 155744 |
| 38776 | 201933925 | 201934547 | 155744 |
| 38777 | 201935414 | 201937575 | 119013, 155744 |
| 38778 | 201980325 | 201984840 | 003402 |
| 38779 | 201989725 | 201991026 | 003402 |
| 38780 | 201991092 | 201995142 | 003402 |
| 38781 | 202008904 | 202009640 | 003402, 226312 |
| 38782 | 202012585 | 202018520 | 003402, 226312 |
| 38783 | 202047212 | 202048154 | 003400 |
| 38784 | 202096594 | 202097444 | |
| 38785 | 202097949 | 202099210 | 064012 |
| 38786 | 202122430 | 202123957 | 064012 |
| 38787 | 202271001 | 202272359 | 082146, 115993 |
| 38788 | 202315452 | 202317835 | 115993, 082146 |
| 38789 | 202332327 | 202332907 | 082146 |
| 38790 | 202506799 | 202508207 | 155755 |
| 38791 | 202644503 | 202646939 | 003393 |
| 38792 | 203050374 | 203051260 | 182329 |
| 38793 | 203052281 | 203052836 | 182329 |
| 38794 | 203102373 | 203103874 | 116030 |
| 38795 | 203130112 | 203131471 | 055044 |
| 38796 | 203142608 | 203143579 | 212309, 055044 |
| 38797 | 203239686 | 203243422 | 204217, 273456 |
| 38798 | 203315440 | 203315961 | 204217 |
| 38799 | 203499219 | 203501221 | 138439 |
| 38800 | 203551676 | 203552808 | 138439 |
| 38801 | 203583260 | 203583731 | 138439 |
| 38802 | 203735902 | 203736634 | 163596 |
| 38803 | 203775408 | 203777469 | 138380, 138442 |
| 38804 | 203878354 | 203880370 | 144426, 138442 |
| 38805 | 203880899 | 203881862 | 144426 |
| 38806 | 203886977 | 203888159 | 144426 |
| 38807 | 204103186 | 204104841 | 119004 |
| 38808 | 204192035 | 204193929 | 138443, 256458 |
| 38809 | 204398233 | 204401287 | 173166 |
| 38810 | 204416435 | 204417067 | |
| 38811 | 204443328 | 204444988 | |
| 38812 | 204469867 | 204470664 | |
| 38813 | 204491773 | 204492560 | |
| 38814 | 204495716 | 204496344 | |
| 38815 | 204496788 | 204498121 | |
| 38816 | 204501411 | 204503051 | |
| 38817 | 204512340 | 204513525 | |
| 38818 | 204514424 | 204516682 | |
| 38819 | 204521020 | 204521915 | |
| 38820 | 204547339 | 204549258 | |
| 38821 | 204675912 | 204678023 | |
| 38822 | 205051795 | 205052883 | |
| 38823 | 205063862 | 205064498 | |
| 38824 | 205179676 | 205180402 | |
| 38825 | 205272363 | 205273001 | |
| 38826 | 205343810 | 205345083 | 237843 |
| 38827 | 205398244 | 205398940 | |
| 38828 | 205407704 | 205408504 | |
| 38829 | 205409549 | 205411070 | 116117 |
| 38830 | 205475520 | 205477157 | 116117 |
| 38831 | 205519760 | 205520215 | 116117 |
| 38832 | 205528890 | 205530613 | 116117 |
| 38833 | 205532362 | 205535634 | 116117 |
| 38834 | 205556500 | 205557133 | 116117 |
| 38835 | 205659757 | 205660493 | 116117 |
| 38836 | 205765143 | 205766295 | 116117 |
| 38837 | 205889637 | 205890388 | 116117 |
| 38838 | 205896629 | 205897356 | 116117 |
| 38839 | 206374905 | 206375536 | 116117 |
| 38840 | 206591155 | 206592547 | 118257 |
| 38841 | 206595280 | 206597054 | 118257 |
| 38842 | 206598056 | 206599696 | 118257 |
| 38843 | 206608472 | 206609892 | 118257 |
| 38844 | 206613235 | 206614469 | 118257 |
| 38845 | 206619441 | 206620128 | 118257 |
| 38846 | 206670770 | 206671619 | |
| 38847 | 206793959 | 206795259 | |
| 38848 | 206839272 | 206840877 | |
| 38849 | 206896733 | 206897356 | 114933 |
| 38850 | 206949009 | 206951686 | 227946, 114933 |
| 38851 | 206979417 | 206980461 | 023228, 231955 |
| 38852 | 207022844 | 207025627 | 114942, 023228 |
| 38853 | 207138975 | 207140456 | 204186 |
| 38854 | 207629260 | 207631208 | 118246, 138400 |
| 38855 | 207832819 | 207834015 | 144410 |
| 38856 | 207993540 | 207994283 | 118263 |
| 38857 | 207997984 | 207999309 | 118263 |
| 38858 | 208021020 | 208021654 | 118263 |
| 38859 | 208026566 | 208032712 | 118263 |
| 38860 | 208198969 | 208199831 | 223725 |
| 38861 | 208280757 | 208281394 | 223725 |
| 38862 | 208393483 | 208396032 | 118260, 223725 |
| 38863 | 208421455 | 208422180 | 118260 |
| 38864 | 208488638 | 208491506 | 144401 |
| 38865 | 208497218 | 208498360 | |
| 38866 | 208541762 | 208542374 | |
| 38867 | 208547973 | 208549905 | |
| 38868 | 208574890 | 208577322 | 163249 |
| 38869 | 208598929 | 208600308 | 163249 |
| 38870 | 208629963 | 208630968 | 163251 |
| 38871 | 208630992 | 208636516 | 163251 |
| 38872 | 208639852 | 208640909 | |
| 38873 | 208646295 | 208647865 | |
| 38874 | 208656682 | 208657819 | |
| 38875 | 208661192 | 208662372 | |
| 38876 | 208663862 | 208664864 | |
| 38877 | 208665622 | 208666909 | |
| 38878 | 208667026 | 208667846 | |
| 38879 | 208669852 | 208672028 | |
| 38880 | 208674763 | 208677505 | |
| 38881 | 208683756 | 208685737 | |
| 38882 | 208692941 | 208695060 | 178385 |
| 38883 | 208697627 | 208698386 | 178385 |
| 38884 | 208701193 | 208701723 | 178385 |
| 38885 | 208709421 | 208714951 | 178385 |
| 38886 | 208729787 | 208730370 | 178385 |
| 38887 | 208732241 | 208736670 | 225111, 178385 |
| 38888 | 208743743 | 208745031 | 178385 |
| 38889 | 208762082 | 208763286 | 178385 |
| 38890 | 208790000 | 208792153 | 178385 |
| 38891 | 208794291 | 208796328 | 178385 |
| 38892 | 208798596 | 208801314 | 178385 |
| 38893 | 208802534 | 208803132 | 178385 |
| 38894 | 208804069 | 208804587 | 178385 |
| 38895 | 208857128 | 208858297 | 178385 |
| 38896 | 208878274 | 208879101 | 178385 |
| 38897 | 208880121 | 208880713 | 178385 |
| 38898 | 208889315 | 208891056 | 178385 |
| 38899 | 209111693 | 209112308 | 138413 |
| 38900 | 209114991 | 209116052 | 138413 |
| 38901 | 209116722 | 209120642 | 231908, 138413 |
| 38902 | 209130381 | 209131908 | 115020, 138413 |
| 38903 | 209213149 | 209213941 | 115020 |
| 38904 | 209219325 | 209220440 | 115020 |
| 38905 | 209223454 | 209224074 | 115020 |
| 38906 | 209233495 | 209236752 | 265454, 144407 |
| 38907 | 210232754 | 210234662 | |
| 38908 | 210247685 | 210248809 | |
| 38909 | 210256177 | 210257950 | |
| 38910 | 210288256 | 210291870 | 078018 |
| 38911 | 210338387 | 210339138 | 078018 |
| 38912 | 210359267 | 210362544 | 078018 |
| 38913 | 210866842 | 210868339 | 197713 |
| 38914 | 211031875 | 211032421 | 144445 |
| 38915 | 211033567 | 211036678 | 263530, 231294, 144445 |
| 38916 | 211054224 | 211055473 | 231294, 115361 |
| 38917 | 211058605 | 211059638 | 231294, 115361 |
| 38918 | 211086265 | 211086959 | 231294, 115361 |
| 38919 | 211088818 | 211091746 | 115361, 231294 |
| 38920 | 211095120 | 211096246 | |
| 38921 | 211340835 | 211341669 | 115365 |
| 38922 | 211403850 | 211406532 | 021826 |
| 38923 | 211411198 | 211412025 | 021826 |
| 38924 | 211412565 | 211414628 | 021826 |
| 38925 | 211420998 | 211428863 | 021826 |
| 38926 | 211526954 | 211527787 | 021826 |
| 38927 | 212272988 | 212273676 | 178568 |
| 38928 | 213316347 | 213317272 | 178568 |
| 38929 | 213776627 | 213777663 | 273118, 223373 |
| 38930 | 213779157 | 213780516 | 273118, 223373 |
| 38931 | 213787931 | 213788502 | 273118 |
| 38932 | 213793230 | 213794217 | 273118 |
| 38933 | 213804663 | 213805360 | 273118 |
| 38934 | 213930617 | 213931166 | 030419 |
| 38935 | 213950674 | 213951678 | 030419 |
| 38936 | 214013894 | 214014710 | 030419 |
| 38937 | 214015015 | 214018064 | 270659, 030419 |
| 38938 | 214018123 | 214018901 | 270659 |
| 38939 | 214028459 | 214029506 | 236193 |
| 38940 | 214051936 | 214053563 | |
| 38941 | 214102339 | 214104451 | |
| 38942 | 214148535 | 214149615 | 144451, 196096 |
| 38943 | 214170981 | 214171916 | 144451 |
| 38944 | 214180068 | 214182162 | 144451 |
| 38945 | 215458410 | 215459001 | 197585 |
| 38946 | 215673715 | 215675167 | 229267, 138376 |
| 38947 | 216176094 | 216177833 | 138363 |
| 38948 | 216275514 | 216276251 | 115414 |
| 38949 | 216278451 | 216284939 | 115414 |
| 38950 | 216285115 | 216285701 | 115414 |
| 38951 | 216296940 | 216302334 | 230695, 115414 |
| 38952 | 216325003 | 216326087 | 225166 |
| 38953 | 216369169 | 216371895 | |
| 38954 | 216545400 | 216546825 | 235770, 237525 |
| 38955 | 216556947 | 216558924 | 235770, 237525 |
| 38956 | 216591735 | 216593702 | 235770 |
| 38957 | 216611200 | 216616327 | 235770 |
| 38958 | 216657927 | 216659449 | 235770 |
| 38959 | 216691068 | 216692220 | 235770 |
| 38960 | 216693248 | 216693664 | 235770 |
| 38961 | 216693847 | 216695806 | 235770 |
| 38962 | 216695892 | 216698081 | 235770 |
| 38963 | 216759019 | 216761087 | |
| 38964 | 216764106 | 216766578 | |
| 38965 | 216767957 | 216769825 | |
| 38966 | 216817466 | 216818525 | 118242 |
| 38967 | 216838665 | 216839737 | 118242 |
| 38968 | 216845990 | 216848821 | 118242 |
| 38969 | 216876473 | 216878681 | 115425, 118242 |
| 38970 | 216921418 | 216923229 | 115425 |
| 38971 | 216936130 | 216936660 | 115425 |
| 38972 | 216942801 | 216944079 | 115425 |
| 38973 | 216945706 | 216947206 | 163449, 115425 |
| 38974 | 216973516 | 216975199 | 079246 |
| 38975 | 216979424 | 216980488 | 079246 |
| 38976 | 216994982 | 216995711 | 079246 |
| 38977 | 217052421 | 217054069 | 079246 |
| 38978 | 217276618 | 217277961 | 138375, 235042 |
| 38979 | 217362532 | 217364912 | 197756, 232485 |
| 38980 | 217387778 | 217388406 | 197756 |
| 38981 | 217403160 | 217405165 | 197756 |
| 38982 | 217435544 | 217438539 | 197756 |
| 38983 | 217480247 | 217481457 | |
| 38984 | 217484249 | 217485149 | |
| 38985 | 217496903 | 217499782 | 115457 |
| 38986 | 217501412 | 217502274 | 115457 |
| 38987 | 217515483 | 217516625 | 115457 |
| 38988 | 217517174 | 217518136 | 115457 |
| 38989 | 217578266 | 217579792 | 236886 |
| 38990 | 217694113 | 217694813 | 236886 |
| 38991 | 217874764 | 217875405 | |
| 38992 | 218195787 | 218197111 | 231672, 233143 |
| 38993 | 218203071 | 218203649 | 231672 |
| 38994 | 218394880 | 218396096 | 231672 |
| 38995 | 218486376 | 218487776 | 231672 |
| 38996 | 218515086 | 218515912 | 231672 |
| 38997 | 218549876 | 218550393 | 231672 |
| 38998 | 218556521 | 218557401 | 231672 |
| 38999 | 218761948 | 218762458 | 079308 |
| 39000 | 218762619 | 218769920 | 079308 |
| 39001 | 218780181 | 218788255 | 079308 |
| 39002 | 218792145 | 218796545 | 079308 |
| 39003 | 218797442 | 218803047 | 079308 |
| 39004 | 218806185 | 218809288 | 079308 |
| 39005 | 218843040 | 218844117 | 223923, 079308 |
| 39006 | 218852408 | 218853738 | 079308, 223923 |
| 39007 | 219080846 | 219083197 | 163466 |
| 39008 | 219133870 | 219135905 | 127838, 127837 |
| 39009 | 219148842 | 219149500 | 127838, 135926 |
| 39010 | 219149733 | 219152359 | 127838, 135926 |
| 39011 | 219154000 | 219159086 | 135926, 127838 |
| 39012 | 219186697 | 219189699 | 127838 |
| 39013 | 219231770 | 219233222 | 225062, 158428 |
| 39014 | 219260204 | 219266383 | 273361, 144579, 018280 |
| 39015 | 219270368 | 219272303 | 144579 |
| 39016 | 219283496 | 219284555 | 127831 |
| 39017 | 219309275 | 219309984 | 127831 |
| 39018 | 219313549 | 219314347 | 127831 |
| 39019 | 219401536 | 219402288 | 135913 |
| 39020 | 219432540 | 219434917 | 144580, 135913 |
| 39021 | 219523260 | 219525105 | 074582, 115568 |
| 39022 | 219536134 | 219537589 | 163482, 163481 |
| 39023 | 219575167 | 219576544 | 135912 |
| 39024 | 219596122 | 219599784 | 135912 |
| 39025 | 219619967 | 219620931 | 135912 |
| 39026 | 219645517 | 219647869 | 135929 |
| 39027 | 219670672 | 219671548 | 135929 |
| 39028 | 219757411 | 219758000 | 135925 |
| 39029 | 219760947 | 219761584 | 135925 |
| 39030 | 219773310 | 219774061 | 228135 |
| 39031 | 219844583 | 219845149 | |
| 39032 | 219866575 | 219868186 | 224090, 181378 |
| 39033 | 219922570 | 219924169 | 264755, 163501 |
| 39034 | 219925281 | 219926052 | |
| 39035 | 219976206 | 219977059 | 213901, 187736 |
| 39036 | 219981455 | 219982076 | 213901, 187736 |
| 39037 | 219989087 | 219992527 | 213901, 187736 |
| 39038 | 220014341 | 220014916 | 213901, 187736 |
| 39039 | 220040259 | 220043787 | 144567, 115649 |
| 39040 | 220070518 | 220073444 | 158552 |
| 39041 | 220082362 | 220083849 | 115657, 198925 |
| 39042 | 220093236 | 220095526 | 163516, 198925 |
| 39043 | 220109388 | 220111273 | 115661, 163521 |
| 39044 | 220113274 | 220115379 | 127824, 115661 |
| 39045 | 220117540 | 220119848 | 243910, 127824 |
| 39046 | 220133957 | 220135442 | 243910, 127824 |
| 39047 | 220136924 | 220140269 | 127824 |
| 39048 | 220142142 | 220145414 | 135924, 127824 |
| 39049 | 220251715 | 220253810 | 229525, 123992 |
| 39050 | 220263396 | 220264169 | 229525, 123992 |
| 39051 | 220271286 | 220272549 | |
| 39052 | 220362601 | 220364229 | 144591, 072195, 268603, |
| | | | 227432 |
| 39053 | 220407352 | 220408961 | 188760, 123989 |
| 39054 | 220435236 | 220436840 | 124006, 123999 |
| 39055 | 220461413 | 220463652 | 144589 |
| 39056 | 221141785 | 221143066 | 239498 |
| 39057 | 221415753 | 221416429 | 236451 |
| 39058 | 222437952 | 222438744 | 272944, 116106 |
| 39059 | 223519066 | 223521906 | 116120 |
| 39060 | 223534858 | 223537385 | 124003 |
| 39061 | 223571615 | 223573561 | 124003 |
| 39062 | 223617952 | 223618620 | |
| 39063 | 223660741 | 223661884 | |
| 39064 | 223702263 | 223702979 | |
| 39065 | 223707201 | 223707673 | |
| 39066 | 223724484 | 223727774 | 123983 |
| 39067 | 223728816 | 223729609 | 123983 |
| 39068 | 223743498 | 223743978 | 123983 |
| 39069 | 223836304 | 223836884 | |
| 39070 | 224330527 | 224331005 | |
| 39071 | 224466874 | 224467344 | 171951 |
| 39072 | 224590263 | 224590710 | |
| 39073 | 224599896 | 224601294 | |
| 39074 | 224623783 | 224626032 | 152056 |
| 39075 | 224663822 | 224664804 | 152056 |
| 39076 | 224679150 | 224680243 | 152056 |
| 39077 | 224701623 | 224702937 | 152056 |
| 39078 | 224720661 | 224721141 | 085449 |
| 39079 | 224724933 | 224727179 | 085449 |
| 39080 | 224759632 | 224760745 | 085449 |
| 39081 | 224772893 | 224774807 | 085449 |
| 39082 | 224781752 | 224782282 | 085449 |
| 39083 | 224794129 | 224796569 | 085449 |
| 39084 | 224805768 | 224807054 | 085449 |
| 39085 | 224807710 | 224810357 | 085449 |
| 39086 | 224821152 | 224823209 | 135900 |
| 39087 | 224859652 | 224861346 | 135919 |
| 39088 | 224873102 | 224874352 | 135919 |
| 39089 | 224887190 | 224888508 | 135919 |
| 39090 | 225015157 | 225016579 | |
| 39091 | 225136732 | 225137593 | |
| 39092 | 225243816 | 225245530 | 124019 |
| 39093 | 225306554 | 225307975 | |
| 39094 | 225313652 | 225314928 | |
| 39095 | 225349804 | 225350570 | 036257 |
| 39096 | 225448662 | 225450601 | 036257 |
| 39097 | 225748863 | 225749400 | 135905 |
| 39098 | 225881741 | 225882628 | 135905 |
| 39099 | 225886768 | 225887480 | 135905 |
| 39100 | 225965642 | 225966157 | |
| 39101 | 226758407 | 226760412 | |
| 39102 | 226859471 | 226860099 | |
| 39103 | 226895688 | 226896452 | |
| 39104 | 226951852 | 226953873 | |
| 39105 | 226978908 | 226981159 | |
| 39106 | 226982590 | 226984825 | |
| 39107 | 227024406 | 227025078 | |
| 39108 | 227032895 | 227033959 | |
| 39109 | 227275663 | 227277736 | |
| 39110 | 227292702 | 227294140 | |
| 39111 | 227304498 | 227306265 | |
| 39112 | 227309338 | 227310722 | |
| 39113 | 227311616 | 227314047 | |
| 39114 | 227342474 | 227343815 | |
| 39115 | 227354515 | 227355252 | |
| 39116 | 227379416 | 227382232 | |
| 39117 | 227388044 | 227391406 | |
| 39118 | 227404467 | 227405893 | |
| 39119 | 227443223 | 227444130 | |
| 39120 | 227445556 | 227446402 | |
| 39121 | 227447601 | 227450463 | |
| 39122 | 227475806 | 227477194 | |
| 39123 | 227483398 | 227484269 | |
| 39124 | 227521782 | 227522928 | |
| 39125 | 227525070 | 227525710 | |
| 39126 | 227597200 | 227599257 | 169047 |
| 39127 | 227602698 | 227604540 | 169047 |
| 39128 | 227618704 | 227621343 | 169047 |
| 39129 | 227624621 | 227626102 | 169047 |
| 39130 | 227654693 | 227666967 | 272622, 169047 |
| 39131 | 227699886 | 227702040 | 144468 |
| 39132 | 227703279 | 227703982 | 144468 |
| 39133 | 227821359 | 227821886 | 144468 |
| 39134 | 227860136 | 227860820 | 144468 |
| 39135 | 227997619 | 227998189 | 081052 |
| 39136 | 228029112 | 228030181 | 169031 |
| 39137 | 228048724 | 228049339 | 169031 |
| 39138 | 228189061 | 228191176 | 168958, 236432 |
| 39139 | 228267426 | 228268212 | |
| 39140 | 228270723 | 228272194 | |
| 39141 | 228280243 | 228280790 | |
| 39142 | 228295074 | 228296207 | |
| 39143 | 228314284 | 228317125 | |
| 39144 | 228330783 | 228332368 | |
| 39145 | 228336474 | 228338727 | 173744, 266382 |
| 39146 | 228368095 | 228368943 | 173744 |
| 39147 | 228517344 | 228521339 | |
| 39148 | 228567688 | 228569320 | 135917 |
| 39149 | 228581928 | 228583344 | 135917 |
| 39150 | 228625785 | 228627316 | |
| 39151 | 228630411 | 228631682 | |
| 39152 | 228660864 | 228661586 | |
| 39153 | 228677518 | 228681036 | 115009 |
| 39154 | 228681257 | 228687036 | 115009 |
| 39155 | 228726982 | 228729269 | |
| 39156 | 228746401 | 228747568 | 123977 |
| 39157 | 229552565 | 229553983 | |
| 39158 | 229703413 | 229707174 | |
| 39159 | 229922330 | 229923316 | 153823 |
| 39160 | 229954646 | 229955884 | 153823 |
| 39161 | 229994454 | 229995112 | 153823 |
| 39162 | 230024276 | 230026035 | 153823 |
| 39163 | 230027992 | 230029944 | 153823 |
| 39164 | 230045303 | 230045826 | 153823 |
| 39165 | 230130350 | 230130912 | 153823 |
| 39166 | 230133365 | 230136404 | 153823 |
| 39167 | 230137807 | 230138967 | |
| 39168 | 230272882 | 230273662 | 187957 |
| 39169 | 230309640 | 230310122 | 187957 |
| 39170 | 230784562 | 230788587 | 153832, 153827 |
| 39171 | 230845364 | 230845934 | 153832 |
| 39172 | 230931662 | 230933398 | 163053 |
| 39173 | 231083937 | 231085309 | 079263, 135899 |
| 39174 | 231191151 | 231192748 | 185404, 079263 |
| 39175 | 231275857 | 231277284 | |
| 39176 | 231280268 | 231281987 | 067066 |
| 39177 | 231288426 | 231289230 | 067066 |
| 39178 | 231289940 | 231290650 | 067066 |
| 39179 | 231367256 | 231368284 | 067066 |
| 39180 | 231368421 | 231369202 | 067066 |
| 39181 | 231376854 | 231378818 | 067066 |
| 39182 | 231383494 | 231387149 | 235419, 067066 |
| 39183 | 231570512 | 231571629 | |
| 39184 | 231577186 | 231579398 | 135932 |
| 39185 | 231712575 | 231713049 | |
| 39186 | 231729282 | 231730366 | 135916 |
| 39187 | 231851420 | 231852396 | 238062 |
| 39188 | 231898528 | 231899532 | |
| 39189 | 231902048 | 231903363 | 204128 |
| 39190 | 231913165 | 231914284 | 204128 |
| 39191 | 231914349 | 231916224 | 204128 |
| 39192 | 231916529 | 231918062 | 261829 |
| 39193 | 231921005 | 231922830 | 173692 |
| 39194 | 231932574 | 231933781 | 173692 |
| 39195 | 231988039 | 231988660 | 135914, 173692 |
| 39196 | 231997923 | 231998839 | 201574, 173692 |
| 39197 | 232046397 | 232046977 | |
| 39198 | 232054705 | 232056053 | |
| 39199 | 232091898 | 232092786 | 135931 |
| 39200 | 232264632 | 232265576 | 156966 |
| 39201 | 232276471 | 232277692 | |
| 39202 | 232278589 | 232279336 | |
| 39203 | 232327936 | 232330478 | 115053 |
| 39204 | 232378423 | 232379954 | 181798 |
| 39205 | 232468752 | 232469841 | |
| 39206 | 232476563 | 232479836 | |
| 39207 | 232526445 | 232527826 | |
| 39208 | 232545041 | 232546865 | |
| 39209 | 232566061 | 232567221 | |
| 39210 | 232570438 | 232576464 | 187514, 269363 |
| 39211 | 232578373 | 232578989 | |
| 39212 | 232645224 | 232646870 | 144524, 156973 |
| 39213 | 232650442 | 232651869 | 156973, 144524 |
| 39214 | 232655747 | 232656373 | 144524 |
| 39215 | 232822515 | 232823097 | |
| 39216 | 232825586 | 232827255 | 144535 |
| 39217 | 232839577 | 232840154 | 144535 |
| 39218 | 233100799 | 233101478 | 144535 |
| 39219 | 233104278 | 233105539 | 144535 |
| 39220 | 233172474 | 233173080 | 144535 |
| 39221 | 233308530 | 233309675 | 223788 |
| 39222 | 233311779 | 233313364 | 223788 |
| 39223 | 233414430 | 233416197 | 135930, 266620, 221944 |
| 39224 | 233453434 | 233454433 | 237126 |
| 39225 | 233457397 | 233458795 | 237126 |
| 39226 | 233470479 | 233471336 | 115468, 237126 |
| 39227 | 233561435 | 233563040 | 204120 |
| 39228 | 233646317 | 233647106 | 204120 |
| 39229 | 233702374 | 233703816 | 204120 |
| 39230 | 233710273 | 233711596 | 204120 |
| 39231 | 233717842 | 233726831 | 182600, 204120 |
| 39232 | 233738247 | 233743355 | 182600 |
| 39233 | 233765963 | 233767153 | 066248 |
| 39234 | 233851888 | 233854292 | 066248 |
| 39235 | 233876225 | 233878637 | 222001, 066248 |
| 39236 | 234159586 | 234162942 | 085978 |
| 39237 | 234257368 | 234258595 | |
| 39238 | 234259156 | 234259942 | 259793 |
| 39239 | 234261614 | 234263474 | 077044, 243637, 259793 |
| 39240 | 234296202 | 234297272 | 077044, 243637 |
| 39241 | 234321096 | 234324648 | 077044 |
| 39242 | 234394379 | 234397921 | 085982 |
| 39243 | 234451037 | 234451980 | 085982 |
| 39244 | 234473917 | 234475751 | 085982 |
| 39245 | 234516698 | 234518562 | |
| 39246 | 234572580 | 234572993 | 242515, 241635 |
| 39247 | 234577157 | 234583671 | 241119, 242515, 241635 |
| 39248 | 234598972 | 234599472 | 242515, 241119, 244122, |
| | | | 241635 |
| 39249 | 234600892 | 234609587 | 224814, 242515, 241119, |
| | | | 167165, 244122, 241635 |
| 39250 | 234613775 | 234616170 | 242515, 241119, 224814, |
| | | | 167165, 244122, 241635 |
| 39251 | 234636840 | 234637732 | 242515, 241119, 167165, |
| | | | 244474, 244122, 241635, |
| | | | 240224 |
| 39252 | 234653937 | 234654850 | 242515, 243135, 241119, |
| | | | 167165, 244474, 244122, |
| | | | 241635, 240224 |
| 39253 | 234665173 | 234665956 | 242515, 243135, 241119, |
| | | | 167165, 244474, 244122, |
| | | | 241635, 240224 |
| 39254 | 234668546 | 234674043 | 242366, 242515, 243135, |
| | | | 241119, 167165, 244474, |
| | | | 244122, 241635, 240224 |
| 39255 | 234726522 | 234727486 | 185038 |
| 39256 | 234731736 | 234732444 | 185038 |
| 39257 | 234776659 | 234777252 | 224287 |
| 39258 | 234874798 | 234875497 | 144481 |
| 39259 | 234881528 | 234892314 | 144481 |
| 39260 | 234898108 | 234899369 | 144481 |
| 39261 | 234990395 | 234991667 | |
| 39262 | 234999861 | 235001192 | |
| 39263 | 235111365 | 235113300 | |
| 39264 | 235197465 | 235198573 | |
| 39265 | 235271491 | 235272085 | |
| 39266 | 235330634 | 235331621 | |
| 39267 | 235404825 | 235405816 | 188042 |
| 39268 | 235464390 | 235466311 | |
| 39269 | 235582438 | 235584278 | |
| 39270 | 235584717 | 235585361 | |
| 39271 | 235586407 | 235586904 | |
| 39272 | 235587699 | 235589672 | |
| 39273 | 235596780 | 235598466 | |
| 39274 | 235602958 | 235603678 | |
| 39275 | 235675409 | 235680845 | |
| 39276 | 235731573 | 235733791 | |
| 39277 | 235747745 | 235749909 | 235726 |
| 39278 | 235751484 | 235753064 | 235726 |
| 39279 | 235753642 | 235755420 | 235726 |
| 39280 | 235767094 | 235769022 | 235726 |
| 39281 | 235770551 | 235774267 | 235726 |
| 39282 | 235779510 | 235780796 | 235726 |
| 39283 | 235787652 | 235791697 | 235726 |
| 39284 | 235859709 | 235864333 | 130147 |
| 39285 | 235887051 | 235893757 | 130147 |
| 39286 | 235904733 | 235911715 | 130147 |
| 39287 | 235916767 | 235918242 | 130147 |
| 39288 | 235929107 | 235932130 | 130147 |
| 39289 | 235933126 | 235937680 | 130147 |
| 39290 | 235957017 | 235960947 | 130147 |
| 39291 | 236007352 | 236008134 | |
| 39292 | 236290816 | 236292576 | |
| 39293 | 236302882 | 236303789 | |
| 39294 | 236401879 | 236403263 | 157985 |
| 39295 | 236403372 | 236404273 | 157985 |
| 39296 | 236419588 | 236420371 | 157985 |
| 39297 | 236423160 | 236423830 | 157985 |
| 39298 | 236577956 | 236578462 | 157985 |
| 39299 | 236617089 | 236619248 | 157985 |
| 39300 | 237476158 | 237477775 | 144476 |
| 39301 | 237477791 | 237478912 | 144476 |
| 39302 | 237637031 | 237638295 | |
| 39303 | 237639989 | 237641580 | |
| 39304 | 237654317 | 237654944 | 232328 |
| 39305 | 237666880 | 237667574 | |
| 39306 | 237690684 | 237692369 | |
| 39307 | 237737053 | 237738109 | |
| 39308 | 237741937 | 237742766 | |
| 39309 | 237744628 | 237745193 | |
| 39310 | 237881046 | 237881927 | |
| 39311 | 237897045 | 237898140 | |
| 39312 | 237932948 | 237936606 | |
| 39313 | 237984410 | 237985294 | 227252 |
| 39314 | 237993389 | 237995376 | 198612, 227252 |
| 39315 | 238036301 | 238037350 | |
| 39316 | 238383237 | 238384719 | |
| 39317 | 238390733 | 238392397 | |
| 39318 | 238395532 | 238396812 | 115648 |
| 39319 | 238410285 | 238411948 | 115648 |
| 39320 | 238421437 | 238422839 | 115648 |
| 39321 | 238448866 | 238450589 | 115648 |
| 39322 | 238459347 | 238460459 | 115648 |
| 39323 | 238490221 | 238491713 | 124839 |
| 39324 | 238497824 | 238500247 | 234949, 124839 |
| 39325 | 238503556 | 238504324 | 234949, 124839 |
| 39326 | 238535413 | 238537669 | 124831 |
| 39327 | 238560767 | 238561294 | 124831 |
| 39328 | 238578113 | 238579299 | 124831 |
| 39329 | 238591775 | 238592644 | 124831 |
| 39330 | 238599801 | 238603092 | 124831 |
| 39331 | 238640214 | 238643287 | 124831 |
| 39332 | 238650408 | 238654133 | 124831 |
| 39333 | 238674948 | 238675263 | 124831 |
| 39334 | 238769386 | 238770387 | 132329 |
| 39335 | 238796981 | 238797468 | 132329 |
| 39336 | 238875061 | 238876817 | 258984, 184182 |
| 39337 | 238968550 | 238970500 | 132330, 258984 |
| 39338 | 238972001 | 238973530 | 258984, 132330 |
| 39339 | 238985921 | 238986599 | 258984, 132330 |
| 39340 | 238988337 | 238989082 | 258984, 132330 |
| 39341 | 239015405 | 239016577 | 144488 |
| 39342 | 239045125 | 239046408 | |
| 39343 | 239052233 | 239052695 | 168427 |
| 39344 | 239076729 | 239079887 | 132323, 178752 |
| 39345 | 239110801 | 239113104 | 132323 |
| 39346 | 239148318 | 239148842 | 225057, 144485 |
| 39347 | 239165032 | 239165797 | 132326 |
| 39348 | 239175732 | 239177281 | 132326 |
| 39349 | 239182470 | 239183761 | 132326 |
| 39350 | 239193085 | 239198489 | 132326 |
| 39351 | 239203003 | 239203891 | |
| 39352 | 239206234 | 239206762 | |
| 39353 | 239208467 | 239209553 | 232306 |
| 39354 | 239214585 | 239215733 | |
| 39355 | 239228832 | 239230212 | 204104 |
| 39356 | 239279258 | 239279908 | 204104 |
| 39357 | 239334331 | 239336949 | 229915, 065802 |
| 39358 | 239346988 | 239351349 | 065802 |
| 39359 | 239505773 | 239506329 | |
| 39360 | 239592871 | 239593573 | |
| 39361 | 239772741 | 239773599 | 233608 |
| 39362 | 240143474 | 240144439 | 068024 |
| 39363 | 240145145 | 240146241 | 068024 |
| 39364 | 240148328 | 240149729 | 068024 |
| 39365 | 240160331 | 240161177 | 068024 |
| 39366 | 240179016 | 240179740 | 068024 |
| 39367 | 240217729 | 240219729 | 068024 |
| 39368 | 240240655 | 240241588 | 068024 |
| 39369 | 240249964 | 240252772 | 068024 |
| 39370 | 240321551 | 240322590 | 068024 |
| 39371 | 240322719 | 240324279 | 222020, 068024 |
| 39372 | 240366663 | 240367346 | |
| 39373 | 240963492 | 240965586 | 130414 |
| 39374 | 241074527 | 241076544 | 172428 |
| 39375 | 241171668 | 241173782 | 227479 |
| 39376 | 241176504 | 241178756 | 227479 |
| 39377 | 241189407 | 241192390 | 227479 |
| 39378 | 241195012 | 241195920 | 227479 |
| 39379 | 241308029 | 241309110 | |
| 39380 | 241309281 | 241310133 | |
| 39381 | 241362128 | 241362783 | |
| 39382 | 241372729 | 241376974 | 063660 |
| 39383 | 241499516 | 241501427 | 188542, 144504 |
| 39384 | 241505170 | 241508951 | 142327, 144504 |
| 39385 | 241518469 | 241520400 | 142327 |
| 39386 | 241524604 | 241527009 | 142330, 260942 |
| 39387 | 241732978 | 241734316 | 130294 |
| 39388 | 241795656 | 241798224 | |
| 39389 | 241807375 | 241809915 | 172482 |
| 39390 | 241813094 | 241814621 | 172482 |
| 39391 | 241815528 | 241816531 | 172482 |
| 39392 | 241828354 | 241829910 | 172478 |
| 39393 | 241925402 | 241927307 | 223991, 226321 |
| 39394 | 241936242 | 241938940 | 162804 |
| 39395 | 242032578 | 242033557 | 122085, 162804 |
| 39396 | 242041293 | 242042811 | 122085 |
| 39397 | 242063221 | 242063694 | 115687 |
| 39398 | 242088401 | 242090650 | 115685, 115687 |
| 39399 | 242156596 | 242157384 | 146205 |
| 39400 | 242157449 | 242158321 | 146205 |
| 39401 | 242194148 | 242195447 | 115677 |
| 39402 | 242210491 | 242213018 | 115677 |
| 39403 | 242224574 | 242225377 | 115677 |
| 39404 | 242253494 | 242256585 | 168385, 115677 |
| 39405 | 242290748 | 242291506 | 223374, 168385 |
| 39406 | 242295410 | 242296648 | 006607 |
| 39407 | 242446564 | 242449194 | 115694 |
| 39408 | 242491698 | 242492656 | 234235 |
| 39409 | 242497790 | 242500801 | 176720, 234235 |
| 39410 | 242549031 | 242550521 | 176946 |
| 39411 | 242575146 | 242577827 | 168397, 176946 |
| 39412 | 242620156 | 242620887 | 168393 |
| 39413 | 242625727 | 242627929 | 168393 |
| 39414 | 242640493 | 242642772 | 168395 |
| 39415 | 242673717 | 242675226 | 180902, 215692 |
| 39416 | 242675363 | 242676024 | 180902 |
| 39417 | 242680359 | 242680882 | 180902 |
| 39418 | 242709302 | 242710417 | |
| 39419 | 242749500 | 242751404 | 204099, 224272 |
| 39420 | 242759943 | 242760674 | |
| 39421 | 242786220 | 242786976 | 235151 |
| 39422 | 242823511 | 242825304 | 233806 |
| 39423 | 242837825 | 242838824 | 233806, 216921 |
| 39424 | 242877501 | 242878156 | 233806 |
| 39425 | 243022993 | 243023388 | 232002 |

The locations of the ODSCs on Chromosome 3 are shown in Table 45.

TABLE 45
ODSC Locations on Chromosome 3

| SEQ ID NO. | Start | End | Overlapping Gene(s) |
|---|---|---|---|
| 39426 | 104708 | 105397 | |
| 39427 | 534265 | 535267 | |
| 39428 | 2893003 | 2894049 | 144619 |
| 39429 | 2903732 | 2905038 | 144619 |
| 39430 | 2911343 | 2915351 | 144619 |
| 39431 | 2937901 | 2938645 | 144619 |
| 39432 | 3167665 | 3169753 | 072756, 091181 |
| 39433 | 3220318 | 3221774 | 113851 |
| 39434 | 4344667 | 4346000 | 170364, 144455 |
| 39435 | 4397099 | 4397758 | 144455 |
| 39436 | 4507863 | 4509571 | 144455 |
| 39437 | 4533776 | 4535667 | 150995, 231249 |
| 39438 | 4575146 | 4576114 | 150995 |
| 39439 | 4607309 | 4608903 | 150995 |
| 39440 | 4761779 | 4763010 | 150995 |
| 39441 | 4764714 | 4767012 | 150995 |
| 39442 | 4770687 | 4771591 | 150995 |
| 39443 | 4782642 | 4784478 | 150995 |
| 39444 | 4790342 | 4794859 | 235947, 150995 |
| 39445 | 4865949 | 4868938 | 235978, 150995 |
| 39446 | 4897738 | 4898539 | 235978 |
| 39447 | 4908049 | 4911242 | 235978 |
| 39448 | 4985242 | 4985640 | 235831 |
| 39449 | 5017643 | 5029840 | 134107, 235831 |
| 39450 | 5034911 | 5038220 | |
| 39451 | 5057429 | 5060621 | |
| 39452 | 5062791 | 5066643 | |
| 39453 | 5067367 | 5069163 | |
| 39454 | 5163632 | 5166019 | 134108 |
| 39455 | 5178105 | 5180623 | 134108 |
| 39456 | 5228228 | 5231461 | 268509, 134109, 233912 |
| 39457 | 5291992 | 5292970 | |
| 39458 | 6969563 | 6970795 | 196277 |
| 39459 | 7545917 | 7547201 | 196277 |
| 39460 | 7660219 | 7660788 | 196277 |
| 39461 | 7682450 | 7683079 | 196277 |
| 39462 | 7688348 | 7689185 | 196277 |
| 39463 | 7692053 | 7692605 | 196277 |
| 39464 | 7781994 | 7782838 | 196277 |
| 39465 | 8018338 | 8020140 | 227110 |
| 39466 | 8483805 | 8487568 | 227110 |
| 39467 | 8501638 | 8503396 | 227110 |
| 39468 | 8523830 | 8527174 | 227110 |
| 39469 | 8529940 | 8533296 | 227110 |
| 39470 | 8538506 | 8541384 | 227110 |
| 39471 | 8541661 | 8544001 | 071282, 227110 |
| 39472 | 8544107 | 8545918 | 227110, 071282 |
| 39473 | 8570808 | 8572841 | 227110, 071282 |
| 39474 | 8573312 | 8574821 | 227110, 071282 |
| 39475 | 8671156 | 8672119 | 125046 |
| 39476 | 8705110 | 8705569 | 125046 |
| 39477 | 8910249 | 8912464 | 070950 |
| 39478 | 8920913 | 8921864 | 070950 |
| 39479 | 8977449 | 8980747 | 070950 |
| 39480 | 9004843 | 9007746 | 070950 |
| 39481 | 9394780 | 9395644 | 196220, 206573, 254485 |
| 39482 | 9403872 | 9405811 | 134077, 196220, 254485, |
| | | | 206573 |
| 39483 | 9436991 | 9441959 | 168137, 206573 |
| 39484 | 9444451 | 9444883 | 168137 |
| 39485 | 9463891 | 9465483 | 168137 |
| 39486 | 9690646 | 9691929 | 163719 |
| 39487 | 9711672 | 9712303 | 163719 |
| 39488 | 9723097 | 9723903 | 163719 |
| 39489 | 9772830 | 9774538 | 156983 |
| 39490 | 9791107 | 9793120 | 114026 |
| 39491 | 9810922 | 9812917 | 134072, 114026 |
| 39492 | 9833342 | 9835743 | 241553, 250151, 171148 |
| 39493 | 9851012 | 9852479 | 214021, 250151 |
| 39494 | 9884852 | 9886524 | 156990, 214021 |
| 39495 | 9903981 | 9905119 | |
| 39496 | 9919866 | 9921753 | 187288 |
| 39497 | 9925595 | 9927208 | |
| 39498 | 9931491 | 9933768 | 171135 |
| 39499 | 9935033 | 9936286 | 171135 |
| 39500 | 9938597 | 9941024 | |
| 39501 | 9942682 | 9943212 | |
| 39502 | 9950771 | 9951764 | 163701 |
| 39503 | 9954011 | 9954631 | 163701 |
| 39504 | 9955722 | 9960551 | 163702, 163701 |
| 39505 | 9974095 | 9976493 | 163703, 163702 |
| 39506 | 9987864 | 9989844 | 230082, 163704 |
| 39507 | 10017600 | 10020235 | 125037 |
| 39508 | 10027569 | 10028760 | 180385, 125037 |
| 39509 | 10052151 | 10052991 | 206567, 125037 |
| 39510 | 10156923 | 10158301 | 254999 |
| 39511 | 10183228 | 10184357 | 134086 |
| 39512 | 10206294 | 10207454 | 134070 |
| 39513 | 10214635 | 10216622 | 134070 |
| 39514 | 10230590 | 10231809 | 134070 |
| 39515 | 10232741 | 10238850 | 134070 |
| 39516 | 10239650 | 10241811 | 134070 |
| 39517 | 10243046 | 10249386 | 134070 |
| 39518 | 10263567 | 10265790 | 134070 |
| 39519 | 10266334 | 10267245 | 134070 |
| 39520 | 10289808 | 10291287 | 272410, 157014 |
| 39521 | 10337793 | 10338784 | 157020 |
| 39522 | 10361762 | 10362998 | 157020 |
| 39523 | 10512175 | 10512744 | 157087 |
| 39524 | 10532286 | 10532912 | 157087 |
| 39525 | 10535871 | 10538144 | 157087 |
| 39526 | 10545764 | 10548273 | 157087 |
| 39527 | 10550037 | 10550876 | 157087 |
| 39528 | 10640614 | 10641790 | 157087 |
| 39529 | 10710761 | 10711900 | 157087 |
| 39530 | 10712892 | 10713488 | 157087 |
| 39531 | 10730292 | 10731015 | 157087 |
| 39532 | 11023706 | 11024293 | |
| 39533 | 11024459 | 11025447 | |
| 39534 | 11029545 | 11030304 | |
| 39535 | 11033740 | 11034840 | 157103 |
| 39536 | 11040106 | 11040863 | 157103 |
| 39537 | 11042679 | 11044771 | 157103 |
| 39538 | 11063201 | 11064182 | 157103 |
| 39539 | 11313801 | 11315013 | 197548 |
| 39540 | 11329845 | 11332398 | 226621, 197548 |
| 39541 | 11487288 | 11487821 | 197548 |
| 39542 | 11539884 | 11540948 | 197548 |
| 39543 | 11683148 | 11685948 | 144560 |
| 39544 | 11701652 | 11702929 | 144560 |
| 39545 | 11732435 | 11733153 | 144560 |
| 39546 | 11760025 | 11762821 | 244030, 144560 |
| 39547 | 11811963 | 11812956 | |
| 39548 | 11887156 | 11888834 | 144559 |
| 39549 | 12179343 | 12180043 | 157152 |
| 39550 | 12197323 | 12197808 | 157152, 157150 |
| 39551 | 12234217 | 12236794 | |
| 39552 | 12266134 | 12266798 | |
| 39553 | 12308685 | 12309859 | |
| 39554 | 12328613 | 12330682 | 132170 |
| 39555 | 12386541 | 12387147 | 132170 |
| 39556 | 12391572 | 12393462 | 132170 |
| 39557 | 12395198 | 12395712 | 132170 |
| 39558 | 12412892 | 12413634 | 132170 |
| 39559 | 12435385 | 12436044 | 132170 |
| 39560 | 12446409 | 12448261 | 132170 |
| 39561 | 12450313 | 12452965 | 132170 |
| 39562 | 12504884 | 12505610 | |
| 39563 | 12525577 | 12527106 | 154743 |
| 39564 | 12594992 | 12595783 | 225526 |
| 39565 | 12597766 | 12599764 | 075975, 225526 |
| 39566 | 12633278 | 12634222 | 132155 |
| 39567 | 12688798 | 12690412 | 132155 |
| 39568 | 12704075 | 12706791 | 132155 |
| 39569 | 12712007 | 12712922 | |
| 39570 | 12804718 | 12805333 | 088726 |
| 39571 | 12881936 | 12883785 | 144713, 207496, 144712 |
| 39572 | 13006137 | 13006933 | 144711 |
| 39573 | 13008380 | 13009659 | 144711 |
| 39574 | 13035817 | 13036960 | 144711 |
| 39575 | 13152925 | 13154530 | |
| 39576 | 13257278 | 13258481 | |
| 39577 | 13258698 | 13262198 | |
| 39578 | 13292659 | 13293657 | |
| 39579 | 13303327 | 13306611 | |
| 39580 | 13318001 | 13318699 | |
| 39581 | 13318900 | 13320007 | |
| 39582 | 13323109 | 13324626 | |
| 39583 | 13405511 | 13406121 | 132182 |
| 39584 | 13454825 | 13455995 | 132182 |
| 39585 | 13460464 | 13462531 | 132182 |
| 39586 | 13512279 | 13512903 | |
| 39587 | 13520729 | 13521839 | 163517, 244502 |
| 39588 | 13526359 | 13527306 | 163517 |
| 39589 | 14160028 | 14160817 | 163528 |
| 39590 | 14164811 | 14167591 | 170876, 163528 |
| 39591 | 14178368 | 14180258 | 170876, 268279 |
| 39592 | 14185107 | 14187293 | 228242, 154767, 170876, |
| | | | 268279 |
| 39593 | 14219211 | 14221043 | 170860, 154767 |
| 39594 | 14243057 | 14244605 | |
| 39595 | 14244702 | 14245633 | |
| 39596 | 14245779 | 14246868 | |
| 39597 | 14294652 | 14295249 | |
| 39598 | 14377753 | 14378490 | |
| 39599 | 14432480 | 14433031 | |
| 39600 | 14629357 | 14630006 | |
| 39601 | 14658905 | 14659491 | |
| 39602 | 14692300 | 14694186 | 154781 |
| 39603 | 14719449 | 14720033 | 131379 |
| 39604 | 14832642 | 14833471 | |
| 39605 | 14843659 | 14846319 | 260902 |
| 39606 | 14856023 | 14856529 | |
| 39607 | 14918370 | 14918946 | 154783 |
| 39608 | 14941504 | 14942413 | 154783 |
| 39609 | 14942667 | 14943411 | 154783 |
| 39610 | 14984347 | 14985028 | 225733 |
| 39611 | 14987874 | 14990576 | 177463, 225733 |
| 39612 | 15031551 | 15032402 | 177463 |
| 39613 | 15090825 | 15091650 | 177463, 131368 |
| 39614 | 15105735 | 15107270 | 131368 |
| 39615 | 15124792 | 15125278 | 131381 |
| 39616 | 15139951 | 15141402 | 131381 |
| 39617 | 15143600 | 15144723 | |
| 39618 | 15148819 | 15150056 | |
| 39619 | 15212466 | 15213169 | 230524 |
| 39620 | 15245456 | 15246019 | 230524 |
| 39621 | 15246769 | 15248984 | 131375, 230524 |
| 39622 | 15340563 | 15343706 | 131370 |
| 39623 | 15361827 | 15362796 | 131370 |
| 39624 | 15364432 | 15365103 | 131370 |
| 39625 | 15366692 | 15367246 | 131370 |
| 39626 | 15367295 | 15368583 | 131370 |
| 39627 | 15372990 | 15374350 | 131370 |
| 39628 | 15428480 | 15429448 | 206562 |
| 39629 | 15450626 | 15451362 | 206562 |
| 39630 | 15468233 | 15470481 | 144597, 206562 |
| 39631 | 15632759 | 15634088 | 131373 |
| 39632 | 15642360 | 15644444 | 169814, 131373 |
| 39633 | 15647589 | 15648673 | 169814 |
| 39634 | 15649274 | 15649888 | 169814 |
| 39635 | 15651392 | 15652302 | 169814 |
| 39636 | 15671574 | 15673633 | 169814 |
| 39637 | 15675486 | 15676157 | 169814 |
| 39638 | 15793610 | 15796497 | 206560 |
| 39639 | 15809208 | 15812020 | 206560 |
| 39640 | 15822575 | 15825069 | 206560 |
| 39641 | 15900065 | 15902610 | 206560 |
| 39642 | 15935181 | 15935729 | |
| 39643 | 15944264 | 15945980 | |
| 39644 | 16096462 | 16098326 | |
| 39645 | 16099744 | 16103622 | |
| 39646 | 16109255 | 16111501 | |
| 39647 | 16131455 | 16132279 | |
| 39648 | 16142239 | 16145633 | |
| 39649 | 16215927 | 16216356 | 131386 |
| 39650 | 16280361 | 16282829 | |
| 39651 | 16305206 | 16307503 | 154814, 154813 |
| 39652 | 16345448 | 16347629 | 154814 |
| 39653 | 16365669 | 16376319 | 154814, 131378 |
| 39654 | 16379735 | 16380929 | 272498, 154814, 131378 |
| 39655 | 16468410 | 16469366 | 131378 |
| 39656 | 16479126 | 16479406 | 131378 |
| 39657 | 16515669 | 16516238 | 131378 |
| 39658 | 16531441 | 16532133 | 131378 |
| 39659 | 16552397 | 16555934 | 131378 |
| 39660 | 16575480 | 16575947 | |
| 39661 | 16655600 | 16656711 | 092345 |
| 39662 | 16876767 | 16877892 | 154822 |
| 39663 | 16879871 | 16880631 | 154822 |
| 39664 | 16905910 | 16906692 | 154822 |
| 39665 | 16925380 | 16927762 | 154822 |
| 39666 | 16948112 | 16949039 | 154822 |
| 39667 | 16958085 | 16961042 | 154822 |
| 39668 | 17034680 | 17035188 | 154822 |
| 39669 | 17055397 | 17056924 | 154822 |
| 39670 | 17068722 | 17070027 | 154822 |
| 39671 | 17080019 | 17082706 | 154822 |
| 39672 | 17216383 | 17217695 | 131374 |
| 39673 | 17414109 | 17414761 | 131374 |
| 39674 | 17426031 | 17426757 | 131374 |
| 39675 | 17462123 | 17462802 | 131374 |
| 39676 | 17519953 | 17520773 | 131374 |
| 39677 | 17679844 | 17682243 | 131374 |
| 39678 | 17782352 | 17785124 | 131374 |
| 39679 | 17790899 | 17791883 | 131374 |
| 39680 | 17794746 | 17795490 | 131374 |
| 39681 | 18464614 | 18468522 | 182568, 131374 |
| 39682 | 18486108 | 18486972 | 228956, 131374, 182568 |
| 39683 | 18510868 | 18511896 | 228956 |
| 39684 | 19258182 | 19258668 | 183960 |
| 39685 | 19943440 | 19943957 | 163576 |
| 39686 | 19986942 | 19989842 | 144566, 163576 |
| 39687 | 20080502 | 20083260 | 114166 |
| 39688 | 20095893 | 20100075 | 114166 |
| 39689 | 20102414 | 20103362 | 114166 |
| 39690 | 20109915 | 20110544 | 114166 |
| 39691 | 20122447 | 20123182 | 114166 |
| 39692 | 20150378 | 20150747 | 114166 |
| 39693 | 20227355 | 20228742 | 129810, 237485 |
| 39694 | 20277655 | 20278625 | |
| 39695 | 20902246 | 20904039 | |
| 39696 | 21662531 | 21663496 | 151789 |
| 39697 | 21686090 | 21688231 | 151789 |
| 39698 | 21713585 | 21715940 | 151789 |
| 39699 | 21746456 | 21748091 | 151789 |
| 39700 | 21759353 | 21760349 | 151789 |
| 39701 | 21942948 | 21943427 | 151789 |
| 39702 | 21961616 | 21962278 | 151789 |
| 39703 | 21996149 | 21997495 | 151789, 223351 |
| 39704 | 22147468 | 22148451 | 151789 |
| 39705 | 22162387 | 22163290 | 151789 |
| 39706 | 22200829 | 22201234 | 151789 |
| 39707 | 23203183 | 23204135 | |
| 39708 | 23213482 | 23213990 | |
| 39709 | 23243765 | 23245865 | 182247, 233153 |
| 39710 | 23708843 | 23710591 | |
| 39711 | 23726970 | 23727999 | |
| 39712 | 23780550 | 23783035 | |
| 39713 | 23846738 | 23848814 | 170142, 223791 |
| 39714 | 23851580 | 23853120 | 170142 |
| 39715 | 23918659 | 23919284 | 170142 |
| 39716 | 23956368 | 23959923 | 174748, 197885 |
| 39717 | 23968902 | 23969501 | 197885 |
| 39718 | 23985893 | 23989413 | 174738, 197885 |
| 39719 | 24005018 | 24005417 | 174738 |
| 39720 | 24009971 | 24010519 | 174738 |
| 39721 | 24088962 | 24089848 | |
| 39722 | 24141791 | 24143320 | 224074 |
| 39723 | 24160705 | 24161128 | 151090 |
| 39724 | 24178495 | 24180087 | 151090 |
| 39725 | 24186506 | 24187834 | 151090 |
| 39726 | 24222528 | 24223246 | 151090 |
| 39727 | 24276589 | 24277274 | 151090 |
| 39728 | 24313652 | 24316191 | 151090 |
| 39729 | 24340997 | 24342112 | 151090 |
| 39730 | 24357572 | 24359285 | 151090 |
| 39731 | 24360053 | 24360963 | 151090 |
| 39732 | 24393120 | 24394087 | 151090 |
| 39733 | 24404623 | 24405372 | 151090 |
| 39734 | 24406316 | 24407306 | 151090 |
| 39735 | 24414642 | 24415177 | 151090 |
| 39736 | 24417339 | 24419793 | 151090 |
| 39737 | 24491410 | 24494497 | 151090 |
| 39738 | 24517461 | 24517884 | 151090 |
| 39739 | 24519894 | 24520531 | 151090 |
| 39740 | 24535047 | 24538556 | 228791, 272554, 151090 |
| 39741 | 24562446 | 24564243 | 265028 |
| 39742 | 24586982 | 24588467 | |
| 39743 | 24589190 | 24590437 | |
| 39744 | 24591770 | 24592737 | |
| 39745 | 24639689 | 24641245 | |
| 39746 | 24745865 | 24746856 | 237838 |
| 39747 | 25463205 | 25463677 | 077092 |
| 39748 | 25468470 | 25470848 | 077092 |
| 39749 | 25499382 | 25500154 | 077092 |
| 39750 | 25661606 | 25662462 | 077097 |
| 39751 | 25704416 | 25706878 | 264219, 077097 |
| 39752 | 25823930 | 25825916 | 151093, 151092 |
| 39753 | 25830153 | 25832548 | 151092, 151093 |
| 39754 | 26479104 | 26481541 | |
| 39755 | 26493737 | 26494301 | |
| 39756 | 26755699 | 26756669 | |
| 39757 | 27180036 | 27180482 | 163491 |
| 39758 | 27319809 | 27321074 | 163491 |
| 39759 | 27401704 | 27402675 | 163491 |
| 39760 | 27467491 | 27468226 | 033867 |
| 39761 | 27472536 | 27473084 | 033867 |
| 39762 | 27490791 | 27491828 | 033867 |
| 39763 | 27524602 | 27526528 | 033867 |
| 39764 | 27562645 | 27563485 | |
| 39765 | 27574279 | 27578083 | |
| 39766 | 27585092 | 27587571 | |
| 39767 | 27626326 | 27626904 | |
| 39768 | 27664525 | 27665246 | |
| 39769 | 27722413 | 27723223 | |
| 39770 | 28282225 | 28284036 | 187118 |
| 39771 | 28389018 | 28391387 | 206559, 163512 |
| 39772 | 28709087 | 28710112 | 228214 |
| 39773 | 28768174 | 28768817 | 228214 |
| 39774 | 28785274 | 28786005 | 228214 |
| 39775 | 29332560 | 29333383 | 144642 |
| 39776 | 29566675 | 29567612 | 144642 |
| 39777 | 29734471 | 29738793 | 144642 |
| 39778 | 29797985 | 29802082 | 144642 |
| 39779 | 30268957 | 30270260 | |
| 39780 | 30275360 | 30276418 | |
| 39781 | 30300150 | 30300908 | |
| 39782 | 30327337 | 30328398 | |
| 39783 | 30553094 | 30553596 | |
| 39784 | 30559395 | 30560721 | |
| 39785 | 30569170 | 30569674 | |
| 39786 | 30580259 | 30581606 | |
| 39787 | 30619073 | 30621093 | |
| 39788 | 30625186 | 30625939 | |
| 39789 | 30638109 | 30639947 | |
| 39790 | 30646577 | 30649396 | 163513 |
| 39791 | 30666609 | 30669295 | 224618, 163513 |
| 39792 | 30685997 | 30686436 | 163513 |
| 39793 | 30699706 | 30701959 | 163513 |
| 39794 | 30761419 | 30764074 | |
| 39795 | 30811884 | 30812408 | 144644 |
| 39796 | 31288926 | 31290185 | |
| 39797 | 31316435 | 31317392 | |
| 39798 | 31418549 | 31419004 | |
| 39799 | 31545655 | 31546228 | |
| 39800 | 31572695 | 31575691 | 163527 |
| 39801 | 31633759 | 31635083 | 163527 |
| 39802 | 32086354 | 32087580 | 252398, 144645 |
| 39803 | 32146014 | 32148940 | 152642 |
| 39804 | 32218514 | 32219872 | |
| 39805 | 32227370 | 32228041 | |
| 39806 | 32257096 | 32258379 | |
| 39807 | 32279055 | 32282211 | 170293, 261572 |
| 39808 | 32290121 | 32291923 | 170293 |
| 39809 | 32390260 | 32391379 | 170293 |
| 39810 | 32432901 | 32434384 | 153551 |
| 39811 | 32442558 | 32443728 | 153551 |
| 39812 | 32511115 | 32511731 | 153551 |
| 39813 | 32535232 | 32536181 | 091317 |
| 39814 | 32543234 | 32544576 | 091317 |
| 39815 | 32611080 | 32613402 | 144635 |
| 39816 | 32671693 | 32672297 | |
| 39817 | 32726277 | 32727516 | 182973 |
| 39818 | 32822292 | 32823406 | |
| 39819 | 32881260 | 32882090 | 206557 |
| 39820 | 33077110 | 33077721 | 170266 |
| 39821 | 33137453 | 33139678 | 170266, 188167 |
| 39822 | 33155188 | 33156232 | 170275 |
| 39823 | 33222678 | 33223211 | 173705 |
| 39824 | 33228316 | 33228805 | 173705 |
| 39825 | 33480631 | 33484104 | 153560 |
| 39826 | 33523641 | 33524392 | |
| 39827 | 33758180 | 33760334 | 163539 |
| 39828 | 33832345 | 33833707 | |
| 39829 | 33839218 | 33841300 | 170248 |
| 39830 | 34052819 | 34054514 | |
| 39831 | 35053182 | 35054264 | |
| 39832 | 35083565 | 35084579 | |
| 39833 | 35226228 | 35226716 | |
| 39834 | 35304761 | 35305337 | |
| 39835 | 35565818 | 35566969 | |
| 39836 | 35569762 | 35571291 | |
| 39837 | 35657761 | 35658545 | |
| 39838 | 35680372 | 35682062 | 172995 |
| 39839 | 35701226 | 35702377 | 172995 |
| 39840 | 35704710 | 35705555 | 172995 |
| 39841 | 35711907 | 35712422 | 172995 |
| 39842 | 35715980 | 35717580 | 172995 |
| 39843 | 35822968 | 35824327 | 172995 |
| 39844 | 35841777 | 35843122 | |
| 39845 | 35985923 | 35987802 | |
| 39846 | 36298542 | 36299179 | |
| 39847 | 36404245 | 36404896 | |
| 39848 | 36934386 | 36935145 | 168016 |
| 39849 | 36949015 | 36951105 | 168016 |
| 39850 | 36953841 | 36954381 | 168016 |
| 39851 | 36960949 | 36962442 | 168016 |
| 39852 | 36973588 | 36976422 | 168016 |
| 39853 | 36984900 | 36986821 | 168016 |
| 39854 | 36995943 | 36996841 | |
| 39855 | 36997879 | 37000699 | |
| 39856 | 37033705 | 37036145 | 076242, 178567 |
| 39857 | 37056136 | 37056534 | 076242 |
| 39858 | 37070495 | 37070956 | 076242 |
| 39859 | 37168736 | 37169273 | 093167 |
| 39860 | 37187147 | 37187506 | 093167 |
| 39861 | 37215212 | 37219233 | 093167 |
| 39862 | 37254449 | 37255061 | |
| 39863 | 37262841 | 37263399 | |
| 39864 | 37270988 | 37271746 | |
| 39865 | 37283847 | 37286817 | 144674, 270194, 176354 |
| 39866 | 37314206 | 37314814 | 144674 |
| 39867 | 37379837 | 37380355 | 144674 |
| 39868 | 37458501 | 37459568 | 198590 |
| 39869 | 37648598 | 37651843 | 144668 |
| 39870 | 37710429 | 37711042 | 144668 |
| 39871 | 37892862 | 37894002 | 235257 |
| 39872 | 37901454 | 37903531 | 144677, 235257 |
| 39873 | 37964030 | 37965022 | 144677 |
| 39874 | 37977688 | 37978219 | 144677 |
| 39875 | 37987375 | 37987883 | 144677 |
| 39876 | 38003484 | 38006779 | 144677 |
| 39877 | 38071745 | 38073758 | |
| 39878 | 38127962 | 38128390 | 008226 |
| 39879 | 38158847 | 38159957 | 008226, 060971 |
| 39880 | 38166358 | 38167561 | 201965, 060971 |
| 39881 | 38169140 | 38169743 | 060971 |
| 39882 | 38173855 | 38176111 | 060971 |
| 39883 | 38177642 | 38181047 | 172936, 060971 |
| 39884 | 38205712 | 38208070 | 172939 |
| 39885 | 38288785 | 38289301 | 172939 |
| 39886 | 38387682 | 38389831 | 093217 |
| 39887 | 38494722 | 38497545 | 114739, 229589 |
| 39888 | 38510147 | 38510838 | 114739 |
| 39889 | 38512531 | 38513457 | 114739 |
| 39890 | 38537521 | 38538544 | 157036 |
| 39891 | 39092631 | 39094389 | 114742 |
| 39892 | 39147851 | 39149751 | 168026, 114745 |
| 39893 | 39160458 | 39161199 | 168026 |
| 39894 | 39167247 | 39167759 | 168026 |
| 39895 | 39187419 | 39190279 | 144655 |
| 39896 | 39191168 | 39196412 | 144655 |
| 39897 | 39207903 | 39209074 | |
| 39898 | 39218340 | 39220238 | |
| 39899 | 39221529 | 39222653 | |
| 39900 | 39328596 | 39330291 | 270367 |
| 39901 | 39333046 | 39334871 | |
| 39902 | 39358420 | 39360869 | |
| 39903 | 39420428 | 39421300 | |
| 39904 | 39424164 | 39426160 | 144659 |
| 39905 | 39447447 | 39449289 | 168028 |
| 39906 | 39573051 | 39573907 | |
| 39907 | 39680487 | 39681327 | |
| 39908 | 39789393 | 39790808 | |
| 39909 | 39793163 | 39794440 | |
| 39910 | 39822982 | 39823535 | |
| 39911 | 39836314 | 39836945 | |
| 39912 | 39850096 | 39852275 | 170011 |
| 39913 | 39941589 | 39942534 | 170011 |
| 39914 | 39951628 | 39953838 | 170011 |
| 39915 | 40128697 | 40129708 | 170011 |
| 39916 | 40308979 | 40310474 | |
| 39917 | 40350253 | 40352279 | 114784 |
| 39918 | 40493555 | 40495401 | 223797 |
| 39919 | 40497976 | 40499837 | 188846 |
| 39920 | 40517770 | 40519325 | 177873 |
| 39921 | 40545863 | 40548108 | 177842 |
| 39922 | 40565434 | 40567317 | 172888 |
| 39923 | 40625131 | 40625701 | |
| 39924 | 40841723 | 40842399 | 231873 |
| 39925 | 40919097 | 40920181 | |
| 39926 | 40952720 | 40954534 | |
| 39927 | 40979989 | 40981688 | |
| 39928 | 41012539 | 41014413 | 233096 |
| 39929 | 41143315 | 41144058 | |
| 39930 | 41239878 | 41242810 | 168036 |
| 39931 | 41281934 | 41282850 | 168036 |
| 39932 | 41309313 | 41310108 | 168038 |
| 39933 | 41316017 | 41317000 | 168038 |
| 39934 | 41668614 | 41669704 | 168038 |
| 39935 | 41722940 | 41723509 | 168038 |
| 39936 | 41729277 | 41730924 | 252409, 168038 |
| 39937 | 41735246 | 41735731 | 168038 |
| 39938 | 42002744 | 42004090 | 168038 |
| 39939 | 42054309 | 42058043 | 182606 |
| 39940 | 42060645 | 42062825 | 182606 |
| 39941 | 42066949 | 42067765 | 182606 |
| 39942 | 42073997 | 42074823 | 182606 |
| 39943 | 42076620 | 42079280 | 182606 |
| 39944 | 42086947 | 42087859 | 182606 |
| 39945 | 42096723 | 42097151 | 182606 |
| 39946 | 42103115 | 42103878 | 182606 |
| 39947 | 42121113 | 42122567 | 182606 |
| 39948 | 42142450 | 42143382 | 182606 |
| 39949 | 42156574 | 42157732 | 182606 |
| 39950 | 42162255 | 42162874 | 182606 |
| 39951 | 42170364 | 42172717 | 182606 |
| 39952 | 42220594 | 42221533 | 182606 |
| 39953 | 42271104 | 42272972 | |
| 39954 | 42585957 | 42587025 | |
| 39955 | 42622681 | 42624370 | 008324, 093183 |
| 39956 | 42632107 | 42633110 | 008324, 093183 |
| 39957 | 42641114 | 42643467 | 114857, 230084, 093183 |
| 39958 | 42673511 | 42674067 | 114857, 230084 |
| 39959 | 42694921 | 42696993 | 114853, 230084 |
| 39960 | 42725414 | 42725840 | |
| 39961 | 42797042 | 42798602 | 244607 |
| 39962 | 42819463 | 42823370 | 181061 |
| 39963 | 42842067 | 42847588 | 235288, 144648, 181061 |
| 39964 | 42856302 | 42857546 | 273328, 235288, 273291, |
| | | | 144648, 240747 |
| 39965 | 42860630 | 42861844 | 273328, 235288, 273291, |
| | | | 144648, 240747 |
| 39966 | 42864234 | 42865841 | 273328, 235288, 273291, |
| | | | 144648, 240747 |
| 39967 | 42867275 | 42873390 | 273328, 235288, 273291, |
| | | | 144648, 240747 |
| 39968 | 42884956 | 42886491 | 273328, 235288, 273291, |
| | | | 144648, 240747 |
| 39969 | 42892555 | 42895116 | 235288, 273328, 273291, |
| | | | 144648, 240747 |
| 39970 | 42895207 | 42898493 | 180432, 273328, 273291, |
| | | | 144648, 240747 |
| 39971 | 42912792 | 42918943 | 180432, 273328, 273291, |
| | | | 144648, 240747 |
| 39972 | 42947122 | 42948626 | 182983, 273328, 273291, |
| | | | 240747 |
| 39973 | 43020357 | 43021660 | 144649, 273291 |
| 39974 | 43042049 | 43043488 | 273291, 144649 |
| 39975 | 43060502 | 43061651 | 273291, 144649 |
| 39976 | 43146912 | 43148058 | 144647 |
| 39977 | 43196957 | 43197762 | |
| 39978 | 43219987 | 43223518 | |
| 39979 | 43249113 | 43249582 | |
| 39980 | 43251206 | 43252662 | 222331 |
| 39981 | 43254631 | 43256096 | |
| 39982 | 43327478 | 43329580 | 163788 |
| 39983 | 43329757 | 43330763 | 163788 |
| 39984 | 43339687 | 43340505 | 163788 |
| 39985 | 43359321 | 43359966 | 163788 |
| 39986 | 43395345 | 43395935 | 163788 |
| 39987 | 43496480 | 43497222 | 160746 |
| 39988 | 43564042 | 43565966 | 160746 |
| 39989 | 43572510 | 43574578 | 160746 |
| 39990 | 43662615 | 43664642 | 160746 |
| 39991 | 43666071 | 43666722 | 160746 |
| 39992 | 43670502 | 43674587 | 160746 |
| 39993 | 43679988 | 43681058 | 160746 |
| 39994 | 43682247 | 43684288 | 160746 |
| 39995 | 43730772 | 43733566 | 011198, 160746 |
| 39996 | 43750513 | 43750986 | 011198 |
| 39997 | 43778088 | 43779224 | |
| 39998 | 43779538 | 43780251 | |
| 39999 | 43793726 | 43797010 | |
| 40000 | 43798664 | 43802018 | |
| 40001 | 43810156 | 43813430 | |
| 40002 | 43925227 | 43926504 | |
| 40003 | 44158732 | 44159365 | 261786 |
| 40004 | 44378802 | 44381612 | 271937, 179152 |
| 40005 | 44397111 | 44397626 | 179152 |
| 40006 | 44400451 | 44401312 | 179152 |
| 40007 | 44480808 | 44482466 | 185219 |
| 40008 | 44518534 | 44519762 | 185219 |
| 40009 | 44551627 | 44553098 | 178917 |
| 40010 | 44666043 | 44667292 | 233509, 236869, 186448 |
| 40011 | 44689936 | 44690957 | 169981, 186448, 236869 |
| 40012 | 44802409 | 44803580 | 163808, 163807 |
| 40013 | 44902695 | 44904656 | 169964, 207783, 163808 |
| 40014 | 44927753 | 44929326 | 163810 |
| 40015 | 44996481 | 44996944 | 163812 |
| 40016 | 45016599 | 45018658 | 075914, 163812 |
| 40017 | 45038016 | 45038627 | 075914 |
| 40018 | 45043597 | 45044194 | 163815, 075914 |
| 40019 | 45241398 | 45242495 | |
| 40020 | 45265357 | 45266008 | 249992 |
| 40021 | 45266460 | 45268163 | 178550, 249992 |
| 40022 | 45429701 | 45430869 | 011376 |
| 40023 | 45521257 | 45522330 | 011376 |
| 40024 | 45528826 | 45529497 | 232455, 011376 |
| 40025 | 45550716 | 45551558 | 232455, 011376 |
| 40026 | 45579517 | 45580613 | 011376 |
| 40027 | 45585262 | 45590466 | 011376 |
| 40028 | 45592737 | 45596202 | |
| 40029 | 45624529 | 45624960 | 144791 |
| 40030 | 45634455 | 45638144 | 144791 |
| 40031 | 45640835 | 45641924 | 144791 |
| 40032 | 45650277 | 45650754 | 144791 |
| 40033 | 45658728 | 45660153 | 144791 |
| 40034 | 45676999 | 45678246 | 144791 |
| 40035 | 45680278 | 45681069 | 144791 |
| 40036 | 45688506 | 45689013 | 144791 |
| 40037 | 45698172 | 45698790 | 144791 |
| 40038 | 45700796 | 45701967 | 144791 |
| 40039 | 45709442 | 45709955 | 144791 |
| 40040 | 45718038 | 45719142 | 144791 |
| 40041 | 45729456 | 45731896 | 211456, 230530 |
| 40042 | 45803506 | 45804369 | 163817 |
| 40043 | 45858933 | 45861118 | |
| 40044 | 45862435 | 45863055 | |
| 40045 | 45867442 | 45868778 | 163818 |
| 40046 | 45883131 | 45884027 | 163818 |
| 40047 | 45902245 | 45903266 | 163818 |
| 40048 | 45994900 | 45995607 | 163820 |
| 40049 | 46026475 | 46031711 | 163820 |
| 40050 | 46035316 | 46037741 | 163820 |
| 40051 | 46048095 | 46048679 | |
| 40052 | 46150516 | 46151144 | |
| 40053 | 46152124 | 46152518 | |
| 40054 | 46155354 | 46157161 | |
| 40055 | 46530069 | 46530886 | |
| 40056 | 46531008 | 46533788 | |
| 40057 | 46537120 | 46543050 | 163825 |
| 40058 | 46599495 | 46601385 | 268324, 163827 |
| 40059 | 46606919 | 46608574 | 163827 |
| 40060 | 46620170 | 46621155 | 241186, 163827 |
| 40061 | 46624866 | 46625323 | 241186 |
| 40062 | 46626500 | 46628916 | 241186 |
| 40063 | 46674192 | 46674795 | |
| 40064 | 46733249 | 46736009 | 178038 |
| 40065 | 46736943 | 46738313 | |
| 40066 | 46886203 | 46888539 | |
| 40067 | 46908191 | 46909542 | 160808 |
| 40068 | 46910402 | 46910913 | 160808 |
| 40069 | 46971614 | 46974148 | 160799 |
| 40070 | 46975308 | 46978524 | 160799 |
| 40071 | 46984152 | 46987212 | 160799 |
| 40072 | 46987324 | 46991393 | 160799 |
| 40073 | 47009777 | 47010913 | 160799 |
| 40074 | 47011860 | 47012904 | 160799 |
| 40075 | 47017240 | 47018858 | 160799 |
| 40076 | 47020270 | 47022205 | 160796, 160799 |
| 40077 | 47057533 | 47058170 | 181555 |
| 40078 | 47138286 | 47139221 | 181555 |
| 40079 | 47181911 | 47183090 | 181555 |
| 40080 | 47204190 | 47206898 | 227398, 181555 |
| 40081 | 47291731 | 47292859 | 239128, 088727 |
| 40082 | 47323056 | 47325159 | 114648, 088727 |
| 40083 | 47420776 | 47423812 | 076201, 260236 |
| 40084 | 47426508 | 47427051 | 076201 |
| 40085 | 47462359 | 47464409 | 114650 |
| 40086 | 47483135 | 47486358 | 114650 |
| 40087 | 47516210 | 47518247 | 114650 |
| 40088 | 47553991 | 47555852 | 163832 |
| 40089 | 47652992 | 47654618 | 173473 |
| 40090 | 47821704 | 47824501 | 173473 |
| 40091 | 47843343 | 47845652 | 132153 |
| 40092 | 47865612 | 47867449 | 132153 |
| 40093 | 47952169 | 47952826 | 047849 |
| 40094 | 48025607 | 48026016 | 047849 |
| 40095 | 48026876 | 48027380 | 047849 |
| 40096 | 48054736 | 48055544 | 047849 |
| 40097 | 48060119 | 48060752 | 047849 |
| 40098 | 48080469 | 48082013 | 270285, 047849 |
| 40099 | 48128090 | 48131101 | 047849 |
| 40100 | 48282238 | 48283394 | 164048 |
| 40101 | 48342101 | 48344075 | 172113 |
| 40102 | 48469787 | 48472113 | 164050 |
| 40103 | 48481167 | 48482701 | 232112, 244380, 164051 |
| 40104 | 48487445 | 48489001 | 164053, 244380 |
| 40105 | 48506545 | 48508533 | 164053, 213689 |
| 40106 | 48509444 | 48510389 | 164054 |
| 40107 | 48513630 | 48515411 | 164054 |
| 40108 | 48541143 | 48542381 | 164054 |
| 40109 | 48594086 | 48594792 | 114268 |
| 40110 | 48646139 | 48647425 | 010256 |
| 40111 | 48672382 | 48673298 | 225697 |
| 40112 | 48700233 | 48701833 | 213672, 228350, 008300 |
| 40113 | 48722548 | 48723754 | 213672 |
| 40114 | 48753795 | 48755087 | 068745 |
| 40115 | 48776702 | 48778566 | 068745 |
| 40116 | 48792009 | 48792525 | 114302 |
| 40117 | 48883958 | 48886108 | 224424, 114302 |
| 40118 | 48901648 | 48902077 | 178537 |
| 40119 | 48934258 | 48936921 | 178537 |
| 40120 | 48955495 | 48957693 | 177479, 221883 |
| 40121 | 49026583 | 49028905 | 178467, 223343 |
| 40122 | 49043771 | 49045619 | 178252, 178467 |
| 40123 | 49051365 | 49051920 | 178252 |
| 40124 | 49055226 | 49056517 | 178149 |
| 40125 | 49057669 | 49060545 | 207605, 178057, 178149 |
| 40126 | 49066100 | 49067046 | 272434, 178035 |
| 40127 | 49130309 | 49132622 | 198218 |
| 40128 | 49141236 | 49142536 | 172053 |
| 40129 | 49157019 | 49159112 | 172037, 172046 |
| 40130 | 49169362 | 49171767 | 172037 |
| 40131 | 49202541 | 49205031 | 177352 |
| 40132 | 49208267 | 49209677 | 185909 |
| 40133 | 49255858 | 49256473 | 173421 |
| 40134 | 49313903 | 49315271 | 114316, 188315 |
| 40135 | 49376516 | 49378272 | 114316 |
| 40136 | 49388312 | 49390491 | |
| 40137 | 49394768 | 49397047 | 067560, 233276 |
| 40138 | 49447747 | 49451385 | 145022, 067560 |
| 40139 | 49456121 | 49457122 | 145020 |
| 40140 | 49457671 | 49460832 | 145029, 235261, 145020 |
| 40141 | 49464988 | 49467451 | 145029 |
| 40142 | 49493721 | 49494331 | |
| 40143 | 49498814 | 49499861 | |
| 40144 | 49506360 | 49508815 | 173402 |
| 40145 | 49535163 | 49539990 | 173402 |
| 40146 | 49557205 | 49557825 | 173402 |
| 40147 | 49572217 | 49573066 | 173402 |
| 40148 | 49573129 | 49574730 | |
| 40149 | 49591089 | 49592592 | 164061, 226913 |
| 40150 | 49703892 | 49704532 | 164061 |
| 40151 | 49705814 | 49706740 | 164061 |
| 40152 | 49710388 | 49712741 | 164062 |
| 40153 | 49760371 | 49762504 | 176095, 176020, 173540 |
| 40154 | 49787661 | 49788743 | 176095 |
| 40155 | 49850519 | 49851999 | 182179 |
| 40156 | 49937248 | 49939861 | 230698, 164078 |
| 40157 | 49942822 | 49944174 | 228008 |
| 40158 | 49965934 | 49967899 | 164077 |
| 40159 | 49977189 | 49978880 | 004534 |
| 40160 | 50125910 | 50127362 | 003756, 004534 |
| 40161 | 50192310 | 50192966 | 001617, 235016 |
| 40162 | 50208811 | 50209714 | 001617 |
| 40163 | 50214711 | 50215203 | 001617 |
| 40164 | 50231632 | 50234153 | 114349 |
| 40165 | 50238907 | 50239665 | |
| 40166 | 50242406 | 50244011 | 188338 |
| 40167 | 50247392 | 50249825 | 188338 |
| 40168 | 50264385 | 50265459 | 114353 |
| 40169 | 50273070 | 50276592 | 114353 |
| 40170 | 50280475 | 50281309 | 114353 |
| 40171 | 50290991 | 50292691 | 114353 |
| 40172 | 50297270 | 50298695 | 230454 |
| 40173 | 50310239 | 50311248 | 012171 |
| 40174 | 50327950 | 50330989 | 186792, 214706 |
| 40175 | 50335973 | 50338995 | 114378, 243477, 186792 |
| 40176 | 50339312 | 50341414 | 114378 |
| 40177 | 50357790 | 50359903 | 068001, 114383 |
| 40178 | 50364481 | 50365907 | 114383 |
| 40179 | 50372951 | 50376005 | 068028 |
| 40180 | 50387824 | 50389502 | 114395, 272104, 114388 |
| 40181 | 50395716 | 50397215 | 114395, 126062, 272104 |
| 40182 | 50604752 | 50605698 | 088543 |
| 40183 | 50606288 | 50607863 | 114735, 088543 |
| 40184 | 50620916 | 50622919 | 114735 |
| 40185 | 50623492 | 50624524 | |
| 40186 | 50624894 | 50626926 | |
| 40187 | 50637542 | 50640589 | |
| 40188 | 50641578 | 50644611 | 114737 |
| 40189 | 50648009 | 50650381 | 114738, 114737 |
| 40190 | 50653297 | 50655456 | 114738 |
| 40191 | 50661810 | 50662493 | 114738 |
| 40192 | 51377577 | 51378513 | 088538 |
| 40193 | 51421143 | 51424464 | 145050, 088538 |
| 40194 | 51428227 | 51430079 | 179837 |
| 40195 | 51532999 | 51534647 | 145041 |
| 40196 | 51569591 | 51570662 | |
| 40197 | 51571822 | 51574151 | |
| 40198 | 51576572 | 51577326 | 164080 |
| 40199 | 51578039 | 51578465 | 164080 |
| 40200 | 51584148 | 51584714 | 164080 |
| 40201 | 51655249 | 51656052 | 164080 |
| 40202 | 51704987 | 51706236 | 164081 |
| 40203 | 51753053 | 51753663 | |
| 40204 | 51975053 | 51977881 | 041880, 114767 |
| 40205 | 51989700 | 51990886 | 180929 |
| 40206 | 52000844 | 52002655 | 114779, 090097, 272762, |
| | | | 254782 |
| 40207 | 52007312 | 52009713 | 243989, 114786, 272762, |
| | | | 254782, 114779, 248487 |
| 40208 | 52015050 | 52019158 | 114779, 248487, 243989, |
| | | | 114786 |
| 40209 | 52019817 | 52020627 | 243989, 114786 |
| 40210 | 52028600 | 52030179 | 162244 |
| 40211 | 52090449 | 52091800 | 164086 |
| 40212 | 52121731 | 52122772 | 164087 |
| 40213 | 52132853 | 52133722 | 164087 |
| 40214 | 52188162 | 52189740 | 164087 |
| 40215 | 52204556 | 52206913 | |
| 40216 | 52208201 | 52213167 | |
| 40217 | 52215044 | 52217407 | |
| 40218 | 52226874 | 52227552 | 242849 |
| 40219 | 52229834 | 52230479 | |
| 40220 | 52231606 | 52233672 | 023330 |
| 40221 | 52238328 | 52239311 | 023330 |
| 40222 | 52239706 | 52240239 | 023330 |
| 40223 | 52244188 | 52245226 | 023330 |
| 40224 | 52245302 | 52246096 | 023330 |
| 40225 | 52250613 | 52251650 | |
| 40226 | 52270258 | 52274012 | 243224, 247596, 239732 |
| 40227 | 52279308 | 52280395 | 164088 |
| 40228 | 52311372 | 52313823 | 164091 |
| 40229 | 52315887 | 52317719 | 164091 |
| 40230 | 52320321 | 52322701 | 242797, 168237, 164091 |
| 40231 | 52323201 | 52326373 | 242797, 168237 |
| 40232 | 52347353 | 52348420 | |
| 40233 | 52357719 | 52358644 | 114841 |
| 40234 | 52443119 | 52445676 | 010318, 163930 |
| 40235 | 52488825 | 52490586 | 010322 |
| 40236 | 52505312 | 52506496 | 010322 |
| 40237 | 52570025 | 52572382 | 168273 |
| 40238 | 52640713 | 52641156 | 163939 |
| 40239 | 52715226 | 52716878 | 163938, 163939 |
| 40240 | 52719199 | 52721000 | 163939, 163938 |
| 40241 | 52738446 | 52741062 | 114902, 016864 |
| 40242 | 52758128 | 52759136 | 114904 |
| 40243 | 52804288 | 52805700 | 114904 |
| 40244 | 52809186 | 52810748 | |
| 40245 | 52811102 | 52814624 | 055957 |
| 40246 | 52827246 | 52829930 | 162267 |
| 40247 | 52847745 | 52848411 | 243696, 055955 |
| 40248 | 52860708 | 52862853 | 243696, 055955 |
| 40249 | 52863220 | 52866834 | 055955, 243696 |
| 40250 | 52878255 | 52880054 | 248592, 213533 |
| 40251 | 52927607 | 52928187 | 248592, 213533 |
| 40252 | 52938036 | 52939035 | 163935 |
| 40253 | 53068876 | 53069635 | 272305, 163935 |
| 40254 | 53072725 | 53073691 | 272305, 163935 |
| 40255 | 53074194 | 53074843 | 272305, 163935 |
| 40256 | 53078364 | 53080948 | 163935, 272305 |
| 40257 | 53106436 | 53107773 | 272305 |
| 40258 | 53118304 | 53119493 | 272305 |
| 40259 | 53126720 | 53127581 | 272305, 163933 |
| 40260 | 53163501 | 53165678 | 163933 |
| 40261 | 53184002 | 53185197 | |
| 40262 | 53193266 | 53195907 | 163932 |
| 40263 | 53253794 | 53254336 | |
| 40264 | 53269261 | 53271369 | 163931 |
| 40265 | 53271959 | 53273118 | 163931 |
| 40266 | 53288271 | 53290541 | 163931 |
| 40267 | 53303189 | 53305177 | |
| 40268 | 53337753 | 53338627 | 162290 |
| 40269 | 53376080 | 53376917 | 162290 |
| 40270 | 53380254 | 53382478 | 162290 |
| 40271 | 53456918 | 53457718 | |
| 40272 | 53498181 | 53499669 | |
| 40273 | 53511984 | 53513612 | |
| 40274 | 53528021 | 53528712 | 157388 |
| 40275 | 53532813 | 53533875 | 157388 |
| 40276 | 53539555 | 53541564 | 157388 |
| 40277 | 53578332 | 53578878 | 157388 |
| 40278 | 53667195 | 53667880 | 157388 |
| 40279 | 53741851 | 53742572 | 157388 |
| 40280 | 53839829 | 53841519 | 157388 |
| 40281 | 53875269 | 53875726 | 016391 |
| 40282 | 53876038 | 53881224 | 056736, 016391 |
| 40283 | 53914779 | 53916692 | 222888, 113812 |
| 40284 | 53919132 | 53920064 | 113811 |
| 40285 | 53924688 | 53927001 | 113811 |
| 40286 | 54356269 | 54356943 | 157445 |
| 40287 | 54390289 | 54390723 | 157445 |
| 40288 | 54393175 | 54393755 | 157445 |
| 40289 | 55436793 | 55437868 | |
| 40290 | 55479617 | 55479987 | |
| 40291 | 55518922 | 55520793 | 114251 |
| 40292 | 55520807 | 55522640 | 244586, 114251 |
| 40293 | 55531179 | 55531981 | 242317 |
| 40294 | 55560318 | 55561029 | 187672 |
| 40295 | 55631210 | 55632267 | 187672 |
| 40296 | 55659825 | 55660310 | 187672 |
| 40297 | 56389427 | 56390875 | 187672 |
| 40298 | 56590376 | 56592086 | 180376 |
| 40299 | 56701860 | 56702673 | 163946 |
| 40300 | 56715903 | 56717691 | 163946 |
| 40301 | 56792492 | 56793225 | 163947 |
| 40302 | 56835576 | 56836879 | 163947 |
| 40303 | 57260795 | 57263208 | 157500 |
| 40304 | 57436119 | 57437176 | 174844 |
| 40305 | 57540843 | 57543325 | 174840 |
| 40306 | 57581823 | 57584643 | 272146, 168374 |
| 40307 | 57677812 | 57679339 | 241933, 174839 |
| 40308 | 57691819 | 57692809 | |
| 40309 | 57737384 | 57738946 | |
| 40310 | 57740930 | 57744108 | 163681 |
| 40311 | 57783427 | 57784431 | 163681 |
| 40312 | 57789170 | 57789985 | 163681 |
| 40313 | 57812530 | 57813691 | 163681 |
| 40314 | 57935346 | 57939389 | |
| 40315 | 57955080 | 57956527 | |
| 40316 | 57959391 | 57961698 | |
| 40317 | 57962192 | 57964807 | |
| 40318 | 57968051 | 57970703 | |
| 40319 | 57993346 | 57997660 | 136068 |
| 40320 | 58011792 | 58012707 | 136068 |
| 40321 | 58025785 | 58026247 | 136068 |
| 40322 | 58033426 | 58040177 | 136068 |
| 40323 | 58043116 | 58047549 | 136068 |
| 40324 | 58050939 | 58053313 | 136068 |
| 40325 | 58053394 | 58054279 | 136068 |
| 40326 | 58056728 | 58057973 | 136068 |
| 40327 | 58087166 | 58089255 | 136068 |
| 40328 | 58096973 | 58097999 | 136068 |
| 40329 | 58103077 | 58103981 | 136068 |
| 40330 | 58120040 | 58120656 | 136068 |
| 40331 | 58129114 | 58129909 | 136068 |
| 40332 | 58140289 | 58140921 | 136068 |
| 40333 | 58141308 | 58142136 | 136068 |
| 40334 | 58146527 | 58151428 | 244161, 136068 |
| 40335 | 58153755 | 58154820 | 244161, 136068 |
| 40336 | 58159637 | 58160426 | |
| 40337 | 58160729 | 58162245 | |
| 40338 | 58166685 | 58167378 | |
| 40339 | 58173275 | 58175822 | |
| 40340 | 58178788 | 58180080 | 163687 |
| 40341 | 58190710 | 58194291 | 163687 |
| 40342 | 58199667 | 58201536 | 163687 |
| 40343 | 58202815 | 58203705 | |
| 40344 | 58216565 | 58217436 | |
| 40345 | 58222385 | 58224517 | 163686 |
| 40346 | 58227098 | 58227851 | 163686 |
| 40347 | 58231557 | 58232922 | 163686 |
| 40348 | 58235093 | 58236252 | 163686 |
| 40349 | 58257430 | 58259057 | 163686 |
| 40350 | 58291664 | 58293274 | 163684 |
| 40351 | 58310906 | 58311769 | |
| 40352 | 58318294 | 58319770 | 168297 |
| 40353 | 58319807 | 58322243 | 168297 |
| 40354 | 58374190 | 58374916 | 168297 |
| 40355 | 58418442 | 58421559 | 168291 |
| 40356 | 58449356 | 58449840 | |
| 40357 | 58456254 | 58458147 | |
| 40358 | 58460527 | 58461352 | |
| 40359 | 58472154 | 58474170 | |
| 40360 | 58477404 | 58478891 | 168301 |
| 40361 | 58505385 | 58506309 | 168306 |
| 40362 | 58507710 | 58509242 | 168306 |
| 40363 | 58512309 | 58512854 | 168306 |
| 40364 | 58518350 | 58519262 | 168306 |
| 40365 | 58521332 | 58523937 | 168306 |
| 40366 | 58549186 | 58551197 | 168309 |
| 40367 | 58653899 | 58654651 | |
| 40368 | 59002591 | 59003863 | 242428, 163689 |
| 40369 | 59026555 | 59027082 | 163689 |
| 40370 | 59034935 | 59036283 | 163689 |
| 40371 | 59305670 | 59306434 | |
| 40372 | 59320240 | 59320949 | |
| 40373 | 59650003 | 59650631 | |
| 40374 | 59842959 | 59843954 | 189283 |
| 40375 | 60071361 | 60072682 | 189283 |
| 40376 | 60406796 | 60408398 | 189283 |
| 40377 | 60584299 | 60584895 | 189283 |
| 40378 | 60826815 | 60827528 | 189283 |
| 40379 | 61141326 | 61141917 | 189283 |
| 40380 | 61159332 | 61159868 | 189283 |
| 40381 | 61180259 | 61181278 | 189283 |
| 40382 | 61207083 | 61208133 | 189283 |
| 40383 | 61235435 | 61236189 | 189283 |
| 40384 | 61236267 | 61237691 | 189283 |
| 40385 | 61249768 | 61250616 | |
| 40386 | 61424009 | 61425031 | |
| 40387 | 61433789 | 61434654 | |
| 40388 | 61503953 | 61504843 | |
| 40389 | 61545743 | 61549119 | 144724 |
| 40390 | 61597790 | 61598628 | 144724 |
| 40391 | 61612902 | 61613968 | 144724 |
| 40392 | 61620251 | 61620875 | 144724 |
| 40393 | 61622743 | 61623902 | 144724 |
| 40394 | 61671976 | 61672613 | 144724 |
| 40395 | 61693687 | 61694254 | 144724 |
| 40396 | 61833557 | 61834497 | 144724 |
| 40397 | 61862901 | 61864175 | 144724 |
| 40398 | 61921424 | 61922432 | 144724 |
| 40399 | 62214098 | 62214702 | 144724 |
| 40400 | 62304432 | 62305329 | 114405, 241472 |
| 40401 | 63848173 | 63851091 | 163635, 163634 |
| 40402 | 63872420 | 63873046 | 163635 |
| 40403 | 63896651 | 63899480 | 163635 |
| 40404 | 63953361 | 63954959 | 163635 |
| 40405 | 63962269 | 63962704 | 163635 |
| 40406 | 64008141 | 64009626 | 163636 |
| 40407 | 64172115 | 64175464 | 226017, 163637 |
| 40408 | 64236345 | 64237619 | 163637 |
| 40409 | 64251905 | 64254079 | 163637 |
| 40410 | 64519202 | 64520124 | 163638 |
| 40411 | 64537256 | 64538354 | 163638 |
| 40412 | 64539813 | 64540372 | 163638 |
| 40413 | 64598636 | 64599520 | 163638 |
| 40414 | 64613383 | 64614043 | 163638 |
| 40415 | 64651551 | 64652289 | 163638 |
| 40416 | 64670552 | 64673177 | 241684, 163638 |
| 40417 | 64673404 | 64674858 | 163638, 241684 |
| 40418 | 64901344 | 64902504 | 241684 |
| 40419 | 64962625 | 64963865 | 241684 |
| 40420 | 65003946 | 65005905 | |
| 40421 | 65131599 | 65132196 | |
| 40422 | 65276949 | 65277624 | |
| 40423 | 65388243 | 65388775 | 151276 |
| 40424 | 65391557 | 65392618 | 151276 |
| 40425 | 65591659 | 65592240 | 151276 |
| 40426 | 65645344 | 65646339 | 151276 |
| 40427 | 65656613 | 65657043 | 151276 |
| 40428 | 65699712 | 65700281 | 151276 |
| 40429 | 65715057 | 65715694 | 151276 |
| 40430 | 65821039 | 65822173 | 151276 |
| 40431 | 65840093 | 65841051 | 151276 |
| 40432 | 65841358 | 65841787 | 151276 |
| 40433 | 65857018 | 65857492 | 151276 |
| 40434 | 65913661 | 65914531 | 272610, 151276 |
| 40435 | 65931357 | 65932662 | 272610, 151276 |
| 40436 | 65938591 | 65940904 | 272610, 151276 |
| 40437 | 65941151 | 65942336 | 151276 |
| 40438 | 65949329 | 65950923 | 151276 |
| 40439 | 65983411 | 65984112 | 151276 |
| 40440 | 65985049 | 65986147 | 151276 |
| 40441 | 65995942 | 65997340 | 151276 |
| 40442 | 66021764 | 66026104 | 151276 |
| 40443 | 66038840 | 66039966 | |
| 40444 | 66059262 | 66059915 | |
| 40445 | 66101164 | 66102510 | |
| 40446 | 66270817 | 66272450 | 144741 |
| 40447 | 66426774 | 66427718 | 144741 |
| 40448 | 66478370 | 66480192 | 144749 |
| 40449 | 66492494 | 66493271 | 144749 |
| 40450 | 66500808 | 66501464 | 144749 |
| 40451 | 66522620 | 66523845 | 144749 |
| 40452 | 66531075 | 66532484 | 144749 |
| 40453 | 66533196 | 66534218 | 144749 |
| 40454 | 66550198 | 66552395 | 144749 |
| 40455 | 66555073 | 66556200 | |
| 40456 | 66644346 | 66644876 | |
| 40457 | 66648522 | 66649378 | |
| 40458 | 66762469 | 66763151 | |
| 40459 | 66858740 | 66860525 | |
| 40460 | 66870235 | 66870853 | |
| 40461 | 66872498 | 66874227 | |
| 40462 | 66902404 | 66903213 | |
| 40463 | 67048441 | 67049573 | 163376 |
| 40464 | 67094493 | 67095375 | |
| 40465 | 67342834 | 67344355 | |
| 40466 | 67345922 | 67347210 | 241884 |
| 40467 | 67452313 | 67452853 | 172340 |
| 40468 | 67566835 | 67567426 | 172340 |
| 40469 | 67590264 | 67591089 | 172340 |
| 40470 | 67643987 | 67644799 | 172340 |
| 40471 | 67674309 | 67675986 | 172340 |
| 40472 | 67684770 | 67686937 | 172340 |
| 40473 | 67694917 | 67701017 | 172340 |
| 40474 | 67702382 | 67705877 | 241316, 172340 |
| 40475 | 67833460 | 67834732 | 241316 |
| 40476 | 67903141 | 67907349 | 241316 |
| 40477 | 67907518 | 67910500 | 241316 |
| 40478 | 67923575 | 67928819 | 241316 |
| 40479 | 67987845 | 67988540 | 241316 |
| 40480 | 67990602 | 67991963 | 241316 |
| 40481 | 68082924 | 68083477 | 183662 |
| 40482 | 68108677 | 68109844 | 183662 |
| 40483 | 69061591 | 69063792 | 244513, 163378 |
| 40484 | 69100380 | 69101892 | 144747, 244513 |
| 40485 | 69128512 | 69130003 | 144744 |
| 40486 | 69131919 | 69132508 | |
| 40487 | 69132998 | 69136219 | 144746 |
| 40488 | 69226952 | 69230276 | 114541 |
| 40489 | 69230769 | 69231761 | 114541 |
| 40490 | 69248318 | 69250576 | 114541 |
| 40491 | 69261502 | 69263011 | 114541 |
| 40492 | 69291488 | 69292933 | 114541 |
| 40493 | 69295653 | 69296178 | 114541 |
| 40494 | 69297306 | 69298271 | 114541 |
| 40495 | 69298402 | 69299651 | 114541 |
| 40496 | 69305001 | 69305626 | 114541 |
| 40497 | 69355180 | 69356290 | 114541 |
| 40498 | 69788062 | 69789368 | 187098 |
| 40499 | 69818508 | 69820852 | 241665, 187098 |
| 40500 | 69836409 | 69837371 | 187098 |
| 40501 | 70313136 | 70314135 | 242120 |
| 40502 | 70931142 | 70931674 | |
| 40503 | 71031751 | 71032371 | 114861 |
| 40504 | 71060465 | 71061386 | 114861 |
| 40505 | 71074642 | 71077121 | 114861 |
| 40506 | 71109683 | 71110119 | 114861 |
| 40507 | 71111593 | 71116530 | 114861 |
| 40508 | 71125060 | 71126999 | 114861 |
| 40509 | 71132700 | 71133323 | 114861 |
| 40510 | 71135558 | 71138854 | 114861 |
| 40511 | 71156787 | 71157677 | 114861 |
| 40512 | 71159604 | 71162689 | 114861 |
| 40513 | 71177440 | 71181251 | 114861 |
| 40514 | 71188798 | 71192871 | 114861 |
| 40515 | 71201701 | 71202461 | 114861 |
| 40516 | 71212444 | 71213800 | 114861 |
| 40517 | 71221878 | 71222713 | 114861 |
| 40518 | 71257479 | 71260412 | 114861 |
| 40519 | 71295061 | 71296834 | 114861 |
| 40520 | 71303554 | 71304340 | 114861 |
| 40521 | 71311452 | 71312658 | 114861 |
| 40522 | 71421243 | 71422176 | 114861 |
| 40523 | 71428911 | 71429699 | 114861 |
| 40524 | 71477333 | 71478907 | 114861 |
| 40525 | 71544405 | 71546239 | 114861 |
| 40526 | 71552185 | 71554713 | 114861 |
| 40527 | 71596823 | 71597463 | 114861 |
| 40528 | 71630133 | 71634169 | 270562, 114861 |
| 40529 | 71744044 | 71745024 | 163412 |
| 40530 | 71773628 | 71774995 | 163412 |
| 40531 | 72103824 | 72104750 | 241163 |
| 40532 | 72149796 | 72150440 | 241163 |
| 40533 | 72172178 | 72174201 | 241163 |
| 40534 | 72176312 | 72177666 | 241163 |
| 40535 | 72193731 | 72195459 | 241163 |
| 40536 | 72199636 | 72201091 | 243083, 241163 |
| 40537 | 72201504 | 72206292 | 243083, 241163 |
| 40538 | 72238530 | 72239957 | 241163 |
| 40539 | 72248685 | 72250077 | 241163 |
| 40540 | 72318426 | 72320432 | |
| 40541 | 72323438 | 72324248 | |
| 40542 | 72355829 | 72357031 | |
| 40543 | 72386551 | 72389434 | |
| 40544 | 72495234 | 72497181 | 163602 |
| 40545 | 72510816 | 72512515 | |
| 40546 | 72531482 | 72532933 | |
| 40547 | 72616454 | 72617713 | |
| 40548 | 72639622 | 72640904 | |
| 40549 | 72642847 | 72643527 | |
| 40550 | 72674303 | 72675311 | |
| 40551 | 72710164 | 72710809 | |
| 40552 | 72839976 | 72840744 | 144736 |
| 40553 | 72869624 | 72871900 | 144736 |
| 40554 | 72896519 | 72897740 | 144736 |
| 40555 | 72956508 | 72957782 | 172986 |
| 40556 | 73002630 | 73003198 | 172986 |
| 40557 | 73016685 | 73017141 | 172986 |
| 40558 | 73021805 | 73022992 | 172986 |
| 40559 | 73045185 | 73047256 | 163605, 172986 |
| 40560 | 73078362 | 73079120 | 163605 |
| 40561 | 73948381 | 73949175 | 242741 |
| 40562 | 74115357 | 74116246 | |
| 40563 | 74457310 | 74459941 | 113805 |
| 40564 | 74461730 | 74464070 | 113805 |
| 40565 | 74484607 | 74485708 | 113805 |
| 40566 | 74595441 | 74596298 | |
| 40567 | 74631990 | 74632538 | |
| 40568 | 74639908 | 74640276 | |
| 40569 | 74648639 | 74649232 | |
| 40570 | 74662159 | 74664686 | |
| 40571 | 74683919 | 74684415 | |
| 40572 | 74692231 | 74693558 | |
| 40573 | 76180543 | 76182241 | 185008 |
| 40574 | 76922061 | 76923473 | 185008 |
| 40575 | 76991970 | 76992722 | 185008 |
| 40576 | 78556142 | 78556989 | |
| 40577 | 78887664 | 78888361 | 169855 |
| 40578 | 78892969 | 78894176 | 169855 |
| 40579 | 78897400 | 78898984 | 169855 |
| 40580 | 78960570 | 78961031 | 169855 |
| 40581 | 78967503 | 78969019 | 169855 |
| 40582 | 79295899 | 79297571 | 169855 |
| 40583 | 79366628 | 79367717 | 169855 |
| 40584 | 79415126 | 79417293 | 169855 |
| 40585 | 79454329 | 79454986 | 169855 |
| 40586 | 79462382 | 79464491 | 169855 |
| 40587 | 81042121 | 81044495 | 243694 |
| 40588 | 81046329 | 81047824 | 243694 |
| 40589 | 81154134 | 81155853 | |
| 40590 | 81298579 | 81299527 | 241593 |
| 40591 | 81491649 | 81494633 | |
| 40592 | 81499605 | 81503438 | |
| 40593 | 81579290 | 81581470 | 114480 |
| 40594 | 81765885 | 81766926 | 114480 |
| 40595 | 81777679 | 81778343 | 114480 |
| 40596 | 81783374 | 81785847 | 114480 |
| 40597 | 81791285 | 81795205 | 114480 |
| 40598 | 81808156 | 81815109 | 242190, 114480 |
| 40599 | 81865644 | 81866534 | |
| 40600 | 81872788 | 81874743 | |
| 40601 | 81918403 | 81918971 | 242009 |
| 40602 | 85055915 | 85057013 | 175161 |
| 40603 | 86842569 | 86844052 | |
| 40604 | 87137745 | 87139238 | |
| 40605 | 87254857 | 87256359 | |
| 40606 | 87275486 | 87278089 | 083937 |
| 40607 | 87317255 | 87317842 | 064835 |
| 40608 | 87553025 | 87553579 | |
| 40609 | 88079213 | 88081391 | |
| 40610 | 88106983 | 88109499 | 229729, 163320 |
| 40611 | 88198279 | 88200112 | 179021, 163320 |
| 40612 | 90183835 | 90186548 | |
| 40613 | 93632102 | 93633396 | 184500 |
| 40614 | 93679989 | 93680956 | 184500 |
| 40615 | 93688172 | 93689189 | 184500 |
| 40616 | 93691370 | 93693377 | 184500 |
| 40617 | 93698750 | 93699526 | 169379 |
| 40618 | 93780989 | 93782783 | 178694, 178700 |
| 40619 | 94159864 | 94160487 | |
| 40620 | 94243052 | 94244281 | |
| 40621 | 95020667 | 95021739 | |
| 40622 | 95985187 | 95986151 | |
| 40623 | 97482913 | 97484043 | 113966 |
| 40624 | 97540173 | 97542007 | 233280 |
| 40625 | 97620239 | 97621175 | 080200 |
| 40626 | 97636588 | 97637256 | 080200 |
| 40627 | 97671065 | 97671578 | 170854 |
| 40628 | 97676362 | 97677047 | 170854 |
| 40629 | 97689910 | 97691692 | 170854 |
| 40630 | 97700615 | 97701584 | |
| 40631 | 97712798 | 97713897 | 183185 |
| 40632 | 98240423 | 98242177 | 248839, 080822, 080819 |
| 40633 | 98293780 | 98294348 | 080819 |
| 40634 | 98311492 | 98312968 | 080819 |
| 40635 | 98411550 | 98412778 | |
| 40636 | 98450404 | 98453554 | 064225, 239445 |
| 40637 | 98460178 | 98461152 | 064225 |
| 40638 | 98468241 | 98470383 | 064225 |
| 40639 | 98496020 | 98496606 | 064225 |
| 40640 | 98502659 | 98505985 | 064225 |
| 40641 | 98529724 | 98530438 | 064225, 057019 |
| 40642 | 98610938 | 98611969 | 057019 |
| 40643 | 98619757 | 98621493 | 239462, 057019 |
| 40644 | 98632517 | 98634084 | 239462 |
| 40645 | 98641551 | 98642115 | 239462 |
| 40646 | 99535712 | 99537717 | 184220 |
| 40647 | 99565189 | 99565928 | 168386, 184220 |
| 40648 | 99593282 | 99597185 | 168386, 184220 |
| 40649 | 99619503 | 99620978 | 168386, 184220 |
| 40650 | 99627095 | 99629911 | 168386, 184220 |
| 40651 | 99638326 | 99640887 | 168386, 184220 |
| 40652 | 99654232 | 99654745 | 168386, 184220 |
| 40653 | 99720517 | 99723199 | 168386, 184220 |
| 40654 | 99759122 | 99762141 | 168386, 184220 |
| 40655 | 99816733 | 99817611 | 168386, 184220 |
| 40656 | 99822446 | 99823353 | 168386, 184220 |
| 40657 | 99827431 | 99827957 | 168386, 184220 |
| 40658 | 99978706 | 99981197 | 036054 |
| 40659 | 100035321 | 100036201 | 036054 |
| 40660 | 100052856 | 100054961 | 114021 |
| 40661 | 100070948 | 100073562 | 114021 |
| 40662 | 100077114 | 100078330 | |
| 40663 | 100118966 | 100121056 | 206535, 154174 |
| 40664 | 100173395 | 100174199 | 206535 |
| 40665 | 100210439 | 100212682 | 181458 |
| 40666 | 100214542 | 100216984 | 181458 |
| 40667 | 100239377 | 100242170 | 181458 |
| 40668 | 100253449 | 100255976 | 181458 |
| 40669 | 100264223 | 100266053 | 181458 |
| 40670 | 100272731 | 100273486 | 181458 |
| 40671 | 100279979 | 100281158 | 181458 |
| 40672 | 100281953 | 100282660 | 181458 |
| 40673 | 100308780 | 100311171 | |
| 40674 | 100321140 | 100323065 | |
| 40675 | 100332602 | 100333427 | 144820 |
| 40676 | 100353414 | 100354743 | 144820 |
| 40677 | 100356010 | 100356586 | 144820 |
| 40678 | 100368314 | 100370739 | 144820 |
| 40679 | 100427627 | 100430018 | 114354 |
| 40680 | 100436452 | 100437411 | 114354 |
| 40681 | 100444751 | 100445381 | 114354 |
| 40682 | 100475186 | 100476028 | 154175 |
| 40683 | 100754899 | 100755808 | |
| 40684 | 100789078 | 100790084 | |
| 40685 | 100796397 | 100797062 | |
| 40686 | 101230969 | 101232929 | 138468 |
| 40687 | 101280228 | 101281454 | 174173 |
| 40688 | 101291841 | 101294559 | 081154 |
| 40689 | 101394584 | 101396749 | 256628, 066422 |
| 40690 | 101403939 | 101406793 | 114391 |
| 40691 | 101442954 | 101444786 | 182504 |
| 40692 | 101545853 | 101548520 | 144802, 144815 |
| 40693 | 101566488 | 101571601 | 144802 |
| 40694 | 101586177 | 101588103 | |
| 40695 | 101597245 | 101599001 | |
| 40696 | 101608607 | 101611310 | |
| 40697 | 101620029 | 101622845 | |
| 40698 | 101647589 | 101648734 | |
| 40699 | 101831554 | 101833528 | 170044 |
| 40700 | 101843050 | 101845216 | 170044 |
| 40701 | 101848446 | 101849950 | 170044 |
| 40702 | 101875214 | 101875935 | 170044 |
| 40703 | 101881823 | 101882398 | 170044 |
| 40704 | 101892979 | 101894095 | 170044 |
| 40705 | 101898198 | 101902062 | 170044 |
| 40706 | 101913602 | 101915487 | 170044 |
| 40707 | 102389610 | 102390527 | 241754 |
| 40708 | 102391785 | 102393420 | 241754 |
| 40709 | 102475383 | 102475969 | |
| 40710 | 102778224 | 102779282 | |
| 40711 | 102796605 | 102797302 | |
| 40712 | 102804233 | 102804903 | |
| 40713 | 103695305 | 103696203 | |
| 40714 | 103845418 | 103847204 | |
| 40715 | 103847317 | 103848176 | |
| 40716 | 105083909 | 105089770 | 170017 |
| 40717 | 105105381 | 105106791 | 170017 |
| 40718 | 105147199 | 105148849 | 170017 |
| 40719 | 105167287 | 105168078 | 170017 |
| 40720 | 105221087 | 105223006 | 170017 |
| 40721 | 105257240 | 105257884 | 170017 |
| 40722 | 105375326 | 105375960 | 114423 |
| 40723 | 105509340 | 105512800 | 114423 |
| 40724 | 105525174 | 105526178 | 114423 |
| 40725 | 105539754 | 105543928 | 114423 |
| 40726 | 105546793 | 105547409 | 114423 |
| 40727 | 105552507 | 105556490 | 114423 |
| 40728 | 105586607 | 105589135 | 114423 |
| 40729 | 105681185 | 105682513 | |
| 40730 | 105716551 | 105717343 | |
| 40731 | 105724800 | 105725798 | |
| 40732 | 105802024 | 105802467 | |
| 40733 | 107139966 | 107140471 | |
| 40734 | 107148657 | 107149100 | |
| 40735 | 107149499 | 107151484 | 273125 |
| 40736 | 107241067 | 107245110 | 114439 |
| 40737 | 107317759 | 107319018 | 114439 |
| 40738 | 107705753 | 107706735 | |
| 40739 | 107718056 | 107718638 | |
| 40740 | 107724919 | 107725833 | |
| 40741 | 107808966 | 107810862 | 196776 |
| 40742 | 107940477 | 107941825 | 114446 |
| 40743 | 108273104 | 108274722 | 163507 |
| 40744 | 108307897 | 108309698 | 198919, 163507 |
| 40745 | 108800600 | 108801376 | 114487 |
| 40746 | 108844527 | 108845425 | |
| 40747 | 108850060 | 108853724 | |
| 40748 | 108873639 | 108875371 | |
| 40749 | 108880211 | 108881133 | |
| 40750 | 109057011 | 109058645 | |
| 40751 | 109080891 | 109083632 | 241257 |
| 40752 | 109362357 | 109362832 | |
| 40753 | 110062054 | 110065091 | |
| 40754 | 110115877 | 110116473 | |
| 40755 | 110131969 | 110134658 | |
| 40756 | 110138801 | 110139645 | |
| 40757 | 110295235 | 110296881 | |
| 40758 | 110342137 | 110342908 | |
| 40759 | 110373169 | 110374618 | |
| 40760 | 110529604 | 110530415 | |
| 40761 | 110789399 | 110792205 | 242242, 177707 |
| 40762 | 110870253 | 110870857 | 177707 |
| 40763 | 110955832 | 110956818 | 177707 |
| 40764 | 111304047 | 111304695 | 153283 |
| 40765 | 111393026 | 111394358 | 240891 |
| 40766 | 111394615 | 111396631 | 240766, 240891 |
| 40767 | 111439390 | 111439966 | 240891 |
| 40768 | 111450974 | 111459659 | 144824, 240891 |
| 40769 | 111481933 | 111484839 | 240891, 144824 |
| 40770 | 111486711 | 111488720 | 240891, 144824 |
| 40771 | 111501393 | 111502521 | 240891, 144824 |
| 40772 | 111519593 | 111523500 | 240891, 144824 |
| 40773 | 111525947 | 111528219 | 240891, 144824 |
| 40774 | 111577309 | 111580895 | 144824 |
| 40775 | 111581608 | 111585104 | 144824 |
| 40776 | 111593114 | 111593853 | 144824 |
| 40777 | 111620846 | 111624814 | 144824 |
| 40778 | 111640953 | 111643883 | 144824 |
| 40779 | 111697335 | 111698714 | 144827 |
| 40780 | 111792793 | 111795017 | 176040 |
| 40781 | 111803659 | 111807200 | 114529 |
| 40782 | 111815533 | 111816849 | 114529 |
| 40783 | 111820068 | 111821866 | 114529 |
| 40784 | 112279382 | 112281714 | 138459, 144848 |
| 40785 | 112709458 | 112710915 | 163607, 272844 |
| 40786 | 112737369 | 112739289 | 240057, 163608 |
| 40787 | 112950427 | 112951233 | 144857 |
| 40788 | 113005640 | 113007416 | 206530, 144857 |
| 40789 | 113224449 | 113225566 | 163611 |
| 40790 | 113232888 | 113234746 | 163611 |
| 40791 | 113251120 | 113251530 | 072858 |
| 40792 | 113345563 | 113346240 | 072858 |
| 40793 | 113381711 | 113383188 | 176542 |
| 40794 | 113404834 | 113405417 | 176542 |
| 40795 | 113414263 | 113416751 | 176542 |
| 40796 | 113417718 | 113418261 | |
| 40797 | 113463868 | 113467039 | 242659, 114573, 121579 |
| 40798 | 113502289 | 113502754 | 114573 |
| 40799 | 113553741 | 113554579 | 178075 |
| 40800 | 113557372 | 113558614 | 178075 |
| 40801 | 113566063 | 113567352 | 178075 |
| 40802 | 113592308 | 113593970 | 178075 |
| 40803 | 113612189 | 113613008 | 178075 |
| 40804 | 113666248 | 113667764 | 184307, 273394 |
| 40805 | 113687787 | 113688237 | 163617 |
| 40806 | 113774608 | 113776683 | 163617, 151576 |
| 40807 | 113871239 | 113872199 | 151577 |
| 40808 | 114084718 | 114085228 | 241560, 181722 |
| 40809 | 114130786 | 114131743 | 181722 |
| 40810 | 114137487 | 114138434 | 181722 |
| 40811 | 114185121 | 114186191 | 242290, 181722 |
| 40812 | 114214681 | 114216593 | 242290, 181722 |
| 40813 | 114223120 | 114226023 | 242290, 181722 |
| 40814 | 114263500 | 114263974 | 181722 |
| 40815 | 114287776 | 114288678 | 181722 |
| 40816 | 114342454 | 114345029 | 181722 |
| 40817 | 114367023 | 114370630 | 181722 |
| 40818 | 114399093 | 114399566 | 181722 |
| 40819 | 114424447 | 114427165 | 181722 |
| 40820 | 114436790 | 114438915 | 181722 |
| 40821 | 114459293 | 114460750 | 181722 |
| 40822 | 114462431 | 114466282 | 181722 |
| 40823 | 114475354 | 114478631 | 181722 |
| 40824 | 114482704 | 114483555 | 181722 |
| 40825 | 114528449 | 114529302 | 181722 |
| 40826 | 114565125 | 114567366 | 181722 |
| 40827 | 114580066 | 114581360 | 181722 |
| 40828 | 114582262 | 114583252 | 181722 |
| 40829 | 114610980 | 114612589 | 181722 |
| 40830 | 114617654 | 114620328 | 181722 |
| 40831 | 114621418 | 114622406 | 181722 |
| 40832 | 114660232 | 114661089 | 181722 |
| 40833 | 114796123 | 114797426 | 181722 |
| 40834 | 114817660 | 114819457 | 242767, 181722 |
| 40835 | 114859740 | 114860557 | 181722 |
| 40836 | 114864713 | 114867715 | 181722 |
| 40837 | 114957974 | 114959061 | |
| 40838 | 115376787 | 115378218 | 241596, 172020 |
| 40839 | 115502290 | 115503829 | |
| 40840 | 115507698 | 115512653 | |
| 40841 | 116936528 | 116937176 | 185565 |
| 40842 | 116937345 | 116938108 | 185565 |
| 40843 | 116970939 | 116972201 | 185565 |
| 40844 | 116980506 | 116981494 | 185565 |
| 40845 | 116996973 | 116998710 | 185565 |
| 40846 | 117572480 | 117573162 | 185565, 239268 |
| 40847 | 117881602 | 117882037 | 243276 |
| 40848 | 118023060 | 118024205 | 243276 |
| 40849 | 118119738 | 118120181 | 243276 |
| 40850 | 118461420 | 118462056 | |
| 40851 | 118536283 | 118537835 | |
| 40852 | 118958587 | 118960783 | 121578, 240254 |
| 40853 | 119012669 | 119014035 | 031081 |
| 40854 | 119041237 | 119042898 | 241155, 031081 |
| 40855 | 119051356 | 119052003 | 031081 |
| 40856 | 119181472 | 119183367 | 176142 |
| 40857 | 119186956 | 119188722 | 163389, 176142 |
| 40858 | 119216359 | 119218261 | 272662, 113845 |
| 40859 | 119297836 | 119299572 | 272967, 144843 |
| 40860 | 119356518 | 119358770 | 121577 |
| 40861 | 119364369 | 119365348 | 121577 |
| 40862 | 119394893 | 119397097 | 138495 |
| 40863 | 119418575 | 119419807 | |
| 40864 | 119420977 | 119422185 | 183833 |
| 40865 | 119422751 | 119423387 | 183833 |
| 40866 | 119435652 | 119436545 | 183833 |
| 40867 | 119499919 | 119502138 | 144852 |
| 40868 | 119512352 | 119516124 | 144852 |
| 40869 | 119544545 | 119545399 | 082701 |
| 40870 | 119646037 | 119646445 | 082701 |
| 40871 | 119811901 | 119815597 | 242622, 082701 |
| 40872 | 119821210 | 119821947 | 242622 |
| 40873 | 119863073 | 119863668 | |
| 40874 | 119878826 | 119879396 | |
| 40875 | 119895843 | 119896685 | 175697 |
| 40876 | 119921758 | 119922263 | 175697 |
| 40877 | 119993466 | 119994037 | 175697 |
| 40878 | 120019719 | 120020502 | |
| 40879 | 120067164 | 120069995 | 240661, 163428 |
| 40880 | 120135746 | 120136942 | 163430 |
| 40881 | 120137670 | 120138264 | 163430 |
| 40882 | 120167432 | 120170123 | 163430 |
| 40883 | 120215189 | 120218296 | |
| 40884 | 120268562 | 120270325 | |
| 40885 | 120270671 | 120272141 | |
| 40886 | 120272397 | 120279015 | |
| 40887 | 120314744 | 120315937 | 065518 |
| 40888 | 120355581 | 120356258 | 113924 |
| 40889 | 120379927 | 120380668 | 113924 |
| 40890 | 120396903 | 120402460 | 113924 |
| 40891 | 120424627 | 120425161 | 144840 |
| 40892 | 120460601 | 120462768 | 153767, 144840 |
| 40893 | 121150819 | 121151479 | 051341 |
| 40894 | 121433929 | 121434398 | 173230 |
| 40895 | 121467449 | 121469510 | 173230 |
| 40896 | 121552854 | 121554079 | 145088, 173226 |
| 40897 | 121715980 | 121716855 | 145103 |
| 40898 | 121717069 | 121717593 | 145103 |
| 40899 | 121937880 | 121938757 | 036828 |
| 40900 | 121974611 | 121975367 | 036828 |
| 40901 | 122071950 | 122072361 | |
| 40902 | 122075267 | 122075884 | |
| 40903 | 122082439 | 122086388 | 160124 |
| 40904 | 122100959 | 122103917 | 114023, 160124 |
| 40905 | 122124725 | 122125672 | 114023 |
| 40906 | 122174548 | 122175668 | 114030 |
| 40907 | 122180438 | 122181406 | 114030 |
| 40908 | 122229613 | 122230206 | 114030 |
| 40909 | 122232343 | 122234112 | 114030 |
| 40910 | 122271910 | 122272628 | 138496 |
| 40911 | 122273952 | 122275261 | 138496 |
| 40912 | 122281616 | 122284229 | 163840, 138496 |
| 40913 | 122291699 | 122292544 | 163840 |
| 40914 | 122328218 | 122329510 | 173200 |
| 40915 | 122387955 | 122389195 | |
| 40916 | 122397756 | 122400945 | 173193 |
| 40917 | 122408424 | 122409269 | 173193 |
| 40918 | 122464122 | 122464623 | 169087 |
| 40919 | 122511996 | 122514764 | 138463, 169087 |
| 40920 | 122661543 | 122662265 | 082684 |
| 40921 | 122755205 | 122755971 | |
| 40922 | 122763783 | 122764766 | |
| 40923 | 122785208 | 122788258 | 065485 |
| 40924 | 122789330 | 122789734 | 065485 |
| 40925 | 122802942 | 122804950 | 065485 |
| 40926 | 122808295 | 122809174 | 065485 |
| 40927 | 122821158 | 122822678 | 065485 |
| 40928 | 122822798 | 122823809 | 065485 |
| 40929 | 122841378 | 122842247 | 065485 |
| 40930 | 122847959 | 122849764 | 065485 |
| 40931 | 122862366 | 122862964 | 065485 |
| 40932 | 122875869 | 122877021 | 065485 |
| 40933 | 122919968 | 122921814 | 121542, 065485 |
| 40934 | 123129924 | 123130830 | 173175 |
| 40935 | 123302822 | 123305288 | 239523, 206527 |
| 40936 | 123321097 | 123321504 | 239523 |
| 40937 | 123322386 | 123325983 | 239523 |
| 40938 | 123370845 | 123373249 | 065534 |
| 40939 | 123404904 | 123409161 | 250174, 065534 |
| 40940 | 123450671 | 123454331 | 065534 |
| 40941 | 123463913 | 123464750 | 065534 |
| 40942 | 123468015 | 123471777 | 065534 |
| 40943 | 123473070 | 123477900 | 065534 |
| 40944 | 123484554 | 123486183 | 065534 |
| 40945 | 123507340 | 123508413 | 065534 |
| 40946 | 123516464 | 123519636 | 065534 |
| 40947 | 123526603 | 123527346 | 065534 |
| 40948 | 123602466 | 123603631 | 065534 |
| 40949 | 123679234 | 123680408 | 175455 |
| 40950 | 123751452 | 123752236 | |
| 40951 | 123882191 | 123883019 | 160145 |
| 40952 | 123966603 | 123968444 | 160145 |
| 40953 | 124081179 | 124084106 | 160145 |
| 40954 | 124100310 | 124100806 | 160145 |
| 40955 | 124114324 | 124115339 | 160145 |
| 40956 | 124248702 | 124249438 | 160145 |
| 40957 | 124270989 | 124271752 | 160145 |
| 40958 | 124303098 | 124304306 | 160145 |
| 40959 | 124311367 | 124312370 | 160145 |
| 40960 | 124317923 | 124320535 | 160145 |
| 40961 | 124325905 | 124326419 | 160145 |
| 40962 | 124338654 | 124340298 | 160145 |
| 40963 | 124353650 | 124354829 | 160145 |
| 40964 | 124373213 | 124374049 | 160145 |
| 40965 | 124383549 | 124384152 | 160145 |
| 40966 | 124388049 | 124388754 | 160145 |
| 40967 | 124417804 | 124420233 | 160145 |
| 40968 | 124448364 | 124450221 | 114491 |
| 40969 | 124492813 | 124494029 | 082781 |
| 40970 | 124499716 | 124500570 | 244286, 082781 |
| 40971 | 124529687 | 124535291 | 082781 |
| 40972 | 124551889 | 124558207 | 082781 |
| 40973 | 124577102 | 124580200 | 082781 |
| 40974 | 124596089 | 124599418 | 082781 |
| 40975 | 124604160 | 124607019 | 082781 |
| 40976 | 124710732 | 124711362 | 173706 |
| 40977 | 124720605 | 124721259 | 173706 |
| 40978 | 124773212 | 124777352 | 252642, 173706 |
| 40979 | 124781008 | 124783145 | |
| 40980 | 124919327 | 124920123 | 221955 |
| 40981 | 124930239 | 124932746 | 221955 |
| 40982 | 125010901 | 125011387 | 163848 |
| 40983 | 125029338 | 125030087 | 229048, 163848 |
| 40984 | 125092736 | 125095610 | 163848 |
| 40985 | 125100403 | 125101494 | |
| 40986 | 125156009 | 125157785 | |
| 40987 | 125238142 | 125239642 | 114520 |
| 40988 | 125312867 | 125315790 | 144909 |
| 40989 | 125340555 | 125341779 | |
| 40990 | 125342829 | 125343859 | |
| 40991 | 125362988 | 125363648 | |
| 40992 | 125366129 | 125366865 | |
| 40993 | 125381430 | 125382080 | |
| 40994 | 125802161 | 125803751 | 250012, 114544 |
| 40995 | 125888773 | 125889845 | 144908 |
| 40996 | 125897549 | 125900763 | 246022, 144908 |
| 40997 | 125902846 | 125904460 | 246022, 144908 |
| 40998 | 125914965 | 125917944 | 144908, 246022 |
| 40999 | 125927295 | 125928392 | 246022 |
| 41000 | 125931453 | 125932744 | |
| 41001 | 125978066 | 125980735 | |
| 41002 | 125984972 | 125987486 | 250934 |
| 41003 | 125988494 | 125990229 | 250934 |
| 41004 | 126005796 | 126007462 | 250934 |
| 41005 | 126019524 | 126020677 | |
| 41006 | 126058923 | 126059474 | |
| 41007 | 126059907 | 126061212 | |
| 41008 | 126069753 | 126077475 | 163884 |
| 41009 | 126085138 | 126087297 | |
| 41010 | 126107575 | 126109000 | |
| 41011 | 126168251 | 126169088 | 070476 |
| 41012 | 126177987 | 126178427 | 070476 |
| 41013 | 126185560 | 126186233 | 070476 |
| 41014 | 126186449 | 126188233 | 070476 |
| 41015 | 126190844 | 126195907 | 070476 |
| 41016 | 126235420 | 126236924 | 159650 |
| 41017 | 126240988 | 126241606 | |
| 41018 | 126242053 | 126244245 | 180767 |
| 41019 | 126246992 | 126250204 | 180767, 180697 |
| 41020 | 126255913 | 126257547 | 180767, 180697 |
| 41021 | 126259259 | 126260276 | 180767, 180697 |
| 41022 | 126372887 | 126374244 | 197763 |
| 41023 | 126422653 | 126423523 | 159685 |
| 41024 | 126700813 | 126702037 | |
| 41025 | 126860520 | 126861206 | |
| 41026 | 127261198 | 127261865 | |
| 41027 | 127262858 | 127264003 | |
| 41028 | 127265270 | 127267172 | |
| 41029 | 127307948 | 127311162 | 163870 |
| 41030 | 127317103 | 127317596 | 073111 |
| 41031 | 127331787 | 127332792 | 073111 |
| 41032 | 127390680 | 127392529 | 114626, 114631 |
| 41033 | 127420588 | 127421560 | 074416 |
| 41034 | 127464903 | 127469519 | 074416 |
| 41035 | 127485010 | 127488793 | 074416 |
| 41036 | 127489446 | 127490067 | 074416 |
| 41037 | 127498378 | 127499610 | 074416 |
| 41038 | 127503471 | 127504900 | 074416 |
| 41039 | 127520494 | 127524226 | 074416 |
| 41040 | 127532977 | 127542784 | 074416 |
| 41041 | 127550491 | 127551204 | |
| 41042 | 127553969 | 127554644 | |
| 41043 | 127680434 | 127681185 | 187715 |
| 41044 | 127728891 | 127729966 | |
| 41045 | 127769519 | 127773452 | 058262 |
| 41046 | 127794102 | 127795614 | 239608, 175792 |
| 41047 | 127798145 | 127801647 | 175792 |
| 41048 | 127802946 | 127803857 | 175792 |
| 41049 | 127812843 | 127814614 | 175792 |
| 41050 | 127817993 | 127818545 | 175792 |
| 41051 | 127841831 | 127843564 | 175792 |
| 41052 | 127870173 | 127871331 | 175792 |
| 41053 | 127871993 | 127872959 | 132394, 175792 |
| 41054 | 127886410 | 127887512 | 132394 |
| 41055 | 128043519 | 128044062 | 132394 |
| 41056 | 128104059 | 128104763 | 132394 |
| 41057 | 128324100 | 128324566 | |
| 41058 | 128326424 | 128327609 | |
| 41059 | 128336311 | 128337997 | |
| 41060 | 128367687 | 128370346 | 163902 |
| 41061 | 128398750 | 128400774 | 163902 |
| 41062 | 128426632 | 128427311 | |
| 41063 | 128444232 | 128446683 | 075785 |
| 41064 | 128596967 | 128599494 | 177646 |
| 41065 | 128639342 | 128640269 | 187695, 114656 |
| 41066 | 128772092 | 128772870 | |
| 41067 | 128795942 | 128796577 | |
| 41068 | 128834997 | 128835978 | 261796, 172780 |
| 41069 | 128837684 | 128841023 | 261796, 172780 |
| 41070 | 128841902 | 128843007 | 273174, 261796 |
| 41071 | 128849873 | 128850861 | 261796, 240682 |
| 41072 | 128879117 | 128882177 | 261796, 240682 |
| 41073 | 128900380 | 128904157 | 169714 |
| 41074 | 128913912 | 128916070 | |
| 41075 | 128930329 | 128931741 | |
| 41076 | 128942792 | 128944829 | |
| 41077 | 128963776 | 128965037 | |
| 41078 | 128967415 | 128969532 | 181789 |
| 41079 | 128976423 | 128976890 | 181789 |
| 41080 | 128997009 | 128999145 | 183624 |
| 41081 | 129000738 | 129002161 | 183624 |
| 41082 | 129028818 | 129029295 | |
| 41083 | 129032562 | 129033339 | |
| 41084 | 129033678 | 129036043 | 206417, 184897 |
| 41085 | 129117481 | 129119363 | 251474 |
| 41086 | 129120929 | 129121594 | 172771 |
| 41087 | 129131202 | 129134462 | 172771 |
| 41088 | 129150843 | 129151478 | 129071 |
| 41089 | 129152078 | 129153144 | 129071 |
| 41090 | 129158008 | 129160137 | 163913, 129071 |
| 41091 | 129160666 | 129161137 | 163913 |
| 41092 | 129180210 | 129181792 | 163913 |
| 41093 | 129253129 | 129254173 | 163914 |
| 41094 | 129344020 | 129347399 | |
| 41095 | 129370321 | 129371685 | 172765 |
| 41096 | 129374350 | 129377928 | 172765 |
| 41097 | 129381676 | 129384297 | 172765 |
| 41098 | 129405690 | 129406186 | 172765 |
| 41099 | 129441284 | 129443798 | 172765 |
| 41100 | 129611273 | 129613409 | 271270, 172765 |
| 41101 | 130421746 | 130422290 | 196455 |
| 41102 | 130445811 | 130446441 | 196455 |
| 41103 | 130451368 | 130452934 | 196455 |
| 41104 | 130464804 | 130466170 | 196455 |
| 41105 | 130466615 | 130467732 | |
| 41106 | 130611657 | 130614166 | 017260 |
| 41107 | 130745026 | 130746340 | 114670, 034533 |
| 41108 | 131068566 | 131069775 | 114670, 250608 |
| 41109 | 131080213 | 131081743 | 246082, 250608 |
| 41110 | 131100089 | 131101653 | 198585, 250608 |
| 41111 | 131220746 | 131222312 | 114686 |
| 41112 | 131262567 | 131263034 | 196353 |
| 41113 | 132135184 | 132137959 | 138246 |
| 41114 | 132140406 | 132141178 | 138246 |
| 41115 | 132355583 | 132357129 | 240303, 113971 |
| 41116 | 132376473 | 132380204 | 240303, 113971, 081307 |
| 41117 | 132440109 | 132441466 | 248724, 113971 |
| 41118 | 132441584 | 132442301 | 248724 |
| 41119 | 133100312 | 133100976 | 144868 |
| 41120 | 133162831 | 133163406 | 170819, 249993 |
| 41121 | 133187836 | 133189014 | 170819 |
| 41122 | 133228171 | 133228781 | |
| 41123 | 133254789 | 133255843 | |
| 41124 | 133259432 | 133260786 | |
| 41125 | 133265669 | 133267646 | |
| 41126 | 133286902 | 133290289 | |
| 41127 | 133291245 | 133294670 | 091527 |
| 41128 | 133380073 | 133381607 | 242337, 163781 |
| 41129 | 133387532 | 133388137 | 242337 |
| 41130 | 133388783 | 133390157 | 242337 |
| 41131 | 133394429 | 133395083 | 242337 |
| 41132 | 133449561 | 133450716 | 242337 |
| 41133 | 133451767 | 133452640 | 242337 |
| 41134 | 133461059 | 133462264 | 242337 |
| 41135 | 133464344 | 133466353 | 091513, 242337 |
| 41136 | 133488273 | 133488787 | 091513 |
| 41137 | 133524170 | 133525690 | 144867 |
| 41138 | 133540560 | 133541213 | 144867 |
| 41139 | 133956256 | 133957016 | 163785 |
| 41140 | 133961172 | 133962378 | 163785 |
| 41141 | 133968411 | 133970089 | 163785 |
| 41142 | 134026914 | 134027904 | |
| 41143 | 134028568 | 134029360 | |
| 41144 | 134029630 | 134034536 | 240006 |
| 41145 | 134044486 | 134047722 | |
| 41146 | 134053091 | 134054138 | |
| 41147 | 134066171 | 134068242 | 260633 |
| 41148 | 134070910 | 134074049 | 214289 |
| 41149 | 134091478 | 134094882 | 114019 |
| 41150 | 134203410 | 134205888 | 182923, 129055 |
| 41151 | 134236043 | 134237433 | 182923 |
| 41152 | 134513564 | 134514473 | 154928 |
| 41153 | 135684389 | 135685712 | 073711 |
| 41154 | 135860084 | 135862218 | 073711 |
| 41155 | 135912688 | 135916728 | 174579 |
| 41156 | 135956845 | 135957540 | |
| 41157 | 135968836 | 135970719 | 114054 |
| 41158 | 136112175 | 136113195 | 118007 |
| 41159 | 136295362 | 136296014 | 118007 |
| 41160 | 136343233 | 136345103 | 118007 |
| 41161 | 136430452 | 136431035 | 118007 |
| 41162 | 136468721 | 136472752 | 261758, 118007 |
| 41163 | 136505185 | 136506776 | |
| 41164 | 136537138 | 136539282 | 168917 |
| 41165 | 136580061 | 136582831 | 158092, 239213 |
| 41166 | 136612036 | 136613907 | 158092 |
| 41167 | 136656731 | 136657614 | 158092 |
| 41168 | 136673995 | 136675879 | 174564 |
| 41169 | 136732314 | 136733925 | |
| 41170 | 136738915 | 136739540 | |
| 41171 | 136800119 | 136801051 | |
| 41172 | 137833919 | 137834884 | 158163 |
| 41173 | 137893135 | 137894221 | 138231 |
| 41174 | 137905726 | 137907921 | 114098 |
| 41175 | 138047857 | 138048924 | 181322 |
| 41176 | 138063475 | 138064002 | |
| 41177 | 138066474 | 138068120 | 158186 |
| 41178 | 138100261 | 138101476 | 158186 |
| 41179 | 138108581 | 138110275 | 158186 |
| 41180 | 138117837 | 138118447 | 158186 |
| 41181 | 138312187 | 138313988 | 114107 |
| 41182 | 138326984 | 138328688 | 158234 |
| 41183 | 138405576 | 138406077 | 051382 |
| 41184 | 138407603 | 138408248 | 051382 |
| 41185 | 138413203 | 138413701 | 051382 |
| 41186 | 138493184 | 138493835 | 051382 |
| 41187 | 138542185 | 138543741 | 051382 |
| 41188 | 138552259 | 138554791 | 051382 |
| 41189 | 138580796 | 138583199 | |
| 41190 | 138633580 | 138635501 | |
| 41191 | 138640500 | 138641098 | |
| 41192 | 138824594 | 138825270 | 175110, 232416 |
| 41193 | 138840257 | 138841543 | 175110, 232416 |
| 41194 | 138845756 | 138846546 | 175110 |
| 41195 | 138892206 | 138893173 | 175110 |
| 41196 | 138969882 | 138970940 | 175110 |
| 41197 | 138985934 | 138987417 | 175110 |
| 41198 | 139016470 | 139017098 | 175110 |
| 41199 | 139021179 | 139021495 | 175110 |
| 41200 | 139062415 | 139063876 | 175110 |
| 41201 | 139107580 | 139109943 | 248932, 184432 |
| 41202 | 139133120 | 139133928 | 248932 |
| 41203 | 139191767 | 139192440 | 248932, 114113 |
| 41204 | 139257640 | 139259229 | 114115, 248932 |
| 41205 | 139396181 | 139397075 | 163864 |
| 41206 | 139625169 | 139625663 | |
| 41207 | 139904787 | 139906314 | 158258 |
| 41208 | 140675576 | 140676442 | 114120 |
| 41209 | 140949788 | 140951566 | 155893 |
| 41210 | 140954395 | 140955348 | 155893 |
| 41211 | 141011556 | 141012112 | 249417, 155893 |
| 41212 | 141012733 | 141014146 | 155893, 249417 |
| 41213 | 141027312 | 141027956 | 249417 |
| 41214 | 141049005 | 141052976 | 249417, 177311 |
| 41215 | 141079507 | 141080542 | 249417, 177311 |
| 41216 | 141085270 | 141088567 | 249417, 177311 |
| 41217 | 141120147 | 141122217 | 177311 |
| 41218 | 141125031 | 141126951 | 177311 |
| 41219 | 141128330 | 141132563 | 177311 |
| 41220 | 141150229 | 141151225 | 177311 |
| 41221 | 141204106 | 141207208 | 155903 |
| 41222 | 141456631 | 141458819 | 114125 |
| 41223 | 141594221 | 141596458 | 069849 |
| 41224 | 141678359 | 141679521 | 114126 |
| 41225 | 141806483 | 141807834 | 114126 |
| 41226 | 141867212 | 141868689 | 114126 |
| 41227 | 141932150 | 141932498 | 175066 |
| 41228 | 141943521 | 141944958 | 175066 |
| 41229 | 141998529 | 141999419 | |
| 41230 | 142037758 | 142038506 | 114127 |
| 41231 | 142118962 | 142120031 | 114127 |
| 41232 | 142165673 | 142167795 | 114127 |
| 41233 | 142178674 | 142179206 | 175054 |
| 41234 | 142296503 | 142298605 | 175054 |
| 41235 | 142305514 | 142306292 | |
| 41236 | 142313885 | 142315986 | 120756 |
| 41237 | 142364060 | 142365285 | 120756 |
| 41238 | 142366626 | 142367125 | 120756 |
| 41239 | 142382694 | 142384079 | 120756 |
| 41240 | 142565543 | 142566205 | 163710 |
| 41241 | 142571733 | 142573087 | 163710 |
| 41242 | 142606957 | 142608513 | 163710 |
| 41243 | 142623210 | 142624401 | |
| 41244 | 142658506 | 142661860 | 243818 |
| 41245 | 142680872 | 142683738 | 163714, 241570, 188582 |
| 41246 | 142695127 | 142696820 | 163714, 241570 |
| 41247 | 142718841 | 142721991 | 268129, 241570, 163714 |
| 41248 | 142783465 | 142784105 | |
| 41249 | 142788922 | 142790505 | |
| 41250 | 143024949 | 143025766 | 181804 |
| 41251 | 143026254 | 143027157 | 181804 |
| 41252 | 143327720 | 143328559 | 181804 |
| 41253 | 143372085 | 143372888 | 181804 |
| 41254 | 143689424 | 143692859 | 181744 |
| 41255 | 143774376 | 143774899 | |
| 41256 | 143823460 | 143824908 | |
| 41257 | 143880547 | 143881645 | |
| 41258 | 143948069 | 143948561 | |
| 41259 | 145236288 | 145236959 | |
| 41260 | 145810038 | 145810470 | 152952, 243415 |
| 41261 | 145811720 | 145812339 | 152952, 243415 |
| 41262 | 145833246 | 145834219 | 152952 |
| 41263 | 145843700 | 145844308 | 152952 |
| 41264 | 145875499 | 145885782 | 152952 |
| 41265 | 145938996 | 145941633 | 114698 |
| 41266 | 145946694 | 145947361 | 114698 |
| 41267 | 145968169 | 145969678 | 114698 |
| 41268 | 145995114 | 145996109 | |
| 41269 | 146025787 | 146027007 | |
| 41270 | 146030948 | 146032209 | |
| 41271 | 146224233 | 146225346 | |
| 41272 | 146228571 | 146229965 | |
| 41273 | 146231179 | 146231924 | |
| 41274 | 146257140 | 146258540 | 188313 |
| 41275 | 146261085 | 146263314 | 188313 |
| 41276 | 146372838 | 146373622 | |
| 41277 | 146902320 | 146903587 | 243620 |
| 41278 | 147109851 | 147113139 | 152977, 174963 |
| 41279 | 148318206 | 148320991 | |
| 41280 | 148414960 | 148417166 | 144891 |
| 41281 | 148427199 | 148428013 | 144891 |
| 41282 | 148444859 | 148445654 | 144891 |
| 41283 | 148453027 | 148456132 | 144891 |
| 41284 | 148628738 | 148629442 | 240521 |
| 41285 | 148661141 | 148661863 | 240521 |
| 41286 | 148692484 | 148693417 | |
| 41287 | 148708970 | 148710471 | 163754 |
| 41288 | 148803627 | 148804752 | 239718, 071794 |
| 41289 | 148846505 | 148848399 | 163755 |
| 41290 | 148895988 | 148899733 | 047457 |
| 41291 | 148931902 | 148937339 | 047457 |
| 41292 | 148937575 | 148945091 | 047457 |
| 41293 | 148953531 | 148954276 | 240216 |
| 41294 | 148961394 | 148966641 | 240216 |
| 41295 | 149015505 | 149018933 | 244468 |
| 41296 | 149028337 | 149028966 | 244468 |
| 41297 | 149032685 | 149035160 | 244468 |
| 41298 | 149056948 | 149059611 | |
| 41299 | 149069318 | 149071021 | |
| 41300 | 149084168 | 149085356 | |
| 41301 | 149090915 | 149092772 | 169908 |
| 41302 | 149092940 | 149096919 | 240541, 169908 |
| 41303 | 149103660 | 149107440 | 240541 |
| 41304 | 149110961 | 149111897 | |
| 41305 | 149117927 | 149122890 | |
| 41306 | 149185746 | 149187475 | |
| 41307 | 149189609 | 149190877 | |
| 41308 | 149191067 | 149196622 | 169903 |
| 41309 | 149254143 | 149255199 | 018408 |
| 41310 | 149260097 | 149260848 | 018408 |
| 41311 | 149265099 | 149266927 | 018408 |
| 41312 | 149269083 | 149269749 | 018408 |
| 41313 | 149298462 | 149300763 | 018408 |
| 41314 | 149339175 | 149339813 | 018408 |
| 41315 | 149374560 | 149377303 | 241313, 018408 |
| 41316 | 149469532 | 149470735 | 114744 |
| 41317 | 149501157 | 149502442 | 206199 |
| 41318 | 149529518 | 149532057 | 082996, 206199 |
| 41319 | 149561252 | 149562475 | 206199, 082996 |
| 41320 | 149686534 | 149689748 | 268175, 070087 |
| 41321 | 149898033 | 149898766 | 243944 |
| 41322 | 149901529 | 149902810 | 243944 |
| 41323 | 149955252 | 149956010 | |
| 41324 | 149968762 | 149970858 | |
| 41325 | 149972460 | 149973828 | |
| 41326 | 149977252 | 149977764 | |
| 41327 | 149985053 | 149988113 | 243550 |
| 41328 | 149996299 | 149997245 | 243550 |
| 41329 | 150034674 | 150036320 | |
| 41330 | 150037425 | 150038781 | |
| 41331 | 150062365 | 150063926 | |
| 41332 | 150065387 | 150067600 | |
| 41333 | 150080278 | 150081417 | |
| 41334 | 150086528 | 150091192 | |
| 41335 | 150093091 | 150094935 | |
| 41336 | 150124580 | 150130131 | 196428 |
| 41337 | 150130168 | 150131067 | 196428 |
| 41338 | 150157403 | 150158044 | 196428 |
| 41339 | 150186826 | 150188502 | |
| 41340 | 150215736 | 150216250 | |
| 41341 | 150262600 | 150265843 | 144895, 120742 |
| 41342 | 150317437 | 150318683 | 120742 |
| 41343 | 150320178 | 150322427 | 198843, 120742 |
| 41344 | 150420853 | 150422317 | 240137, 163645 |
| 41345 | 150453967 | 150458144 | |
| 41346 | 150458465 | 150463301 | 181788 |
| 41347 | 150477725 | 150482516 | 244265, 181788 |
| 41348 | 150483637 | 150484606 | |
| 41349 | 150487276 | 150490203 | |
| 41350 | 150491284 | 150491878 | |
| 41351 | 150802791 | 150803677 | 144893 |
| 41352 | 150863624 | 150864597 | 144893 |
| 41353 | 150915036 | 150917602 | 174946, 144893 |
| 41354 | 150927941 | 150929813 | 144893 |
| 41355 | 151022728 | 151024081 | 138271, 144893 |
| 41356 | 151151658 | 151152728 | 152580, 144893 |
| 41357 | 151347590 | 151350388 | |
| 41358 | 151483415 | 151484763 | 242908 |
| 41359 | 151487611 | 151489188 | 240602, 242908 |
| 41360 | 151531296 | 151534856 | 114771, 242908 |
| 41361 | 151554661 | 151555870 | 242908 |
| 41362 | 151576610 | 151578869 | 242908 |
| 41363 | 151582354 | 151583109 | 242908 |
| 41364 | 151693607 | 151694884 | |
| 41365 | 151976133 | 151977277 | 152601 |
| 41366 | 151985774 | 151988889 | 229619, 152601 |
| 41367 | 152016173 | 152022044 | 152601 |
| 41368 | 152024637 | 152026475 | 152601 |
| 41369 | 152032500 | 152033239 | 152601 |
| 41370 | 152045900 | 152047644 | 152601 |
| 41371 | 152184483 | 152185092 | 243305 |
| 41372 | 152363572 | 152364273 | |
| 41373 | 152367766 | 152369246 | 244545 |
| 41374 | 152675096 | 152675700 | |
| 41375 | 152678373 | 152679551 | |
| 41376 | 152855477 | 152857747 | |
| 41377 | 152857917 | 152859058 | |
| 41378 | 152877415 | 152881395 | 181467, 244268 |
| 41379 | 153317172 | 153318074 | 238755 |
| 41380 | 153838809 | 153842059 | 243069, 114790 |
| 41381 | 153843240 | 153844031 | 114790 |
| 41382 | 153846745 | 153848044 | 114790 |
| 41383 | 153852800 | 153854011 | 114790 |
| 41384 | 153857532 | 153858314 | 114790 |
| 41385 | 153960380 | 153962662 | 114790 |
| 41386 | 153968968 | 153970156 | 114790 |
| 41387 | 154041165 | 154043044 | 174953 |
| 41388 | 154797391 | 154798901 | 196549 |
| 41389 | 154867131 | 154867817 | 196549 |
| 41390 | 154949513 | 154952118 | |
| 41391 | 155020116 | 155021134 | |
| 41392 | 155289688 | 155290361 | 114805 |
| 41393 | 155323070 | 155324394 | 114805 |
| 41394 | 155455424 | 155456046 | 114805 |
| 41395 | 155466601 | 155467213 | |
| 41396 | 155523268 | 155524393 | 174928 |
| 41397 | 155570765 | 155573449 | 169359 |
| 41398 | 155587488 | 155589610 | 163655 |
| 41399 | 155663905 | 155664402 | |
| 41400 | 156252177 | 156253701 | 169282 |
| 41401 | 156271758 | 156273949 | 114850 |
| 41402 | 156304471 | 156305257 | |
| 41403 | 156316126 | 156316708 | |
| 41404 | 156323250 | 156324899 | |
| 41405 | 156345844 | 156346565 | |
| 41406 | 156382921 | 156383483 | |
| 41407 | 156385743 | 156387208 | |
| 41408 | 156391288 | 156394108 | 243926, 163659 |
| 41409 | 156530155 | 156531497 | 240875 |
| 41410 | 156533149 | 156537244 | 240875 |
| 41411 | 156542998 | 156545505 | 197980 |
| 41412 | 156787475 | 156788953 | |
| 41413 | 156804168 | 156808490 | 241135, 241544, 243629 |
| 41414 | 156837192 | 156841779 | 243629 |
| 41415 | 156844091 | 156855628 | |
| 41416 | 156876276 | 156879630 | 163660 |
| 41417 | 156891470 | 156894354 | 222675, 243176 |
| 41418 | 157216847 | 157217637 | 197415 |
| 41419 | 157827390 | 157828847 | 174891 |
| 41420 | 157885920 | 157886307 | 174891 |
| 41421 | 158138491 | 158139223 | 174891 |
| 41422 | 158189293 | 158190094 | 174891 |
| 41423 | 158361928 | 158363188 | 168827 |
| 41424 | 158429560 | 158430224 | 118849 |
| 41425 | 158443086 | 158444069 | 118849 |
| 41426 | 158449568 | 158450922 | 240207, 118855, 118849 |
| 41427 | 158518529 | 158520816 | 272247, 118855 |
| 41428 | 158884621 | 158885419 | 214216, 250588 |
| 41429 | 158904707 | 158905966 | 214216, 250588 |
| 41430 | 159168341 | 159169770 | 250588 |
| 41431 | 159338641 | 159339019 | 250588 |
| 41432 | 159339742 | 159340323 | 250588 |
| 41433 | 159352614 | 159353184 | 250588 |
| 41434 | 159386712 | 159387305 | 250588 |
| 41435 | 159479181 | 159484677 | 241211, 250588 |
| 41436 | 159500696 | 159501244 | 250588 |
| 41437 | 159504344 | 159506614 | 250588 |
| 41438 | 159506766 | 159508536 | 250588 |
| 41439 | 159516561 | 159517702 | 250588 |
| 41440 | 159535967 | 159536946 | 250588 |
| 41441 | 159540936 | 159543581 | 250588 |
| 41442 | 159546006 | 159547976 | 250588 |
| 41443 | 159570485 | 159571578 | 151967, 250588 |
| 41444 | 159571589 | 159573809 | 151967, 250588 |
| 41445 | 159589349 | 159591675 | 151967, 250588 |
| 41446 | 159631285 | 159632227 | 244040 |
| 41447 | 159645830 | 159648024 | 244040 |
| 41448 | 159681908 | 159682524 | 244040 |
| 41449 | 159706085 | 159707356 | 168811, 244040 |
| 41450 | 159770044 | 159771280 | 244040 |
| 41451 | 159824786 | 159825919 | 244040 |
| 41452 | 159846665 | 159848535 | 244040 |
| 41453 | 159928611 | 159929175 | 244040 |
| 41454 | 160008677 | 160009878 | 068885, 248710 |
| 41455 | 160090463 | 160094128 | 068885, 248710 |
| 41456 | 160116299 | 160119801 | 113810, 068885, 248710 |
| 41457 | 160281905 | 160284962 | 229320, 186432 |
| 41458 | 160317819 | 160318256 | |
| 41459 | 160334539 | 160336629 | |
| 41460 | 160340093 | 160340952 | |
| 41461 | 160383230 | 160384429 | |
| 41462 | 160384912 | 160385790 | |
| 41463 | 160389850 | 160392072 | |
| 41464 | 160394021 | 160396876 | 179674 |
| 41465 | 160415638 | 160416215 | |
| 41466 | 160472081 | 160474418 | 163590, 260572 |
| 41467 | 160513699 | 160515638 | 163590 |
| 41468 | 160518593 | 160519997 | 163590 |
| 41469 | 160525711 | 160527104 | 163590 |
| 41470 | 160527922 | 160528843 | 163590 |
| 41471 | 160653102 | 160653651 | 163590 |
| 41472 | 160665832 | 160666632 | 163590 |
| 41473 | 160759887 | 160760624 | 163590 |
| 41474 | 160822311 | 160823343 | 169251, 169255 |
| 41475 | 160881046 | 160881535 | 169251 |
| 41476 | 160888737 | 160889332 | 169251 |
| 41477 | 160935070 | 160935798 | 169251 |
| 41478 | 160938254 | 160940397 | 169251 |
| 41479 | 160942428 | 160943072 | 169251 |
| 41480 | 160982234 | 160983489 | |
| 41481 | 161124529 | 161125259 | |
| 41482 | 161132427 | 161134790 | |
| 41483 | 161138402 | 161139415 | |
| 41484 | 161161740 | 161162537 | 240567 |
| 41485 | 161193522 | 161194368 | |
| 41486 | 161510954 | 161511514 | |
| 41487 | 161538825 | 161539733 | 240354 |
| 41488 | 163291074 | 163291568 | |
| 41489 | 164203761 | 164204293 | |
| 41490 | 164216642 | 164217167 | |
| 41491 | 164702317 | 164703483 | 090402 |
| 41492 | 164850948 | 164851814 | |
| 41493 | 164930042 | 164930872 | 244128 |
| 41494 | 165241667 | 165242074 | 244128 |
| 41495 | 165353719 | 165354944 | 244128 |
| 41496 | 165355626 | 165356418 | 244128 |
| 41497 | 165360182 | 165362025 | 244128 |
| 41498 | 165467363 | 165468331 | |
| 41499 | 165470478 | 165471869 | |
| 41500 | 165500895 | 165501786 | 114200 |
| 41501 | 165522965 | 165524599 | 114200 |
| 41502 | 165554059 | 165555477 | 114200 |
| 41503 | 166547465 | 166548351 | |
| 41504 | 167021627 | 167022323 | 169064 |
| 41505 | 167097756 | 167098455 | 169064 |
| 41506 | 167434624 | 167435305 | 114209 |
| 41507 | 167451718 | 167453775 | 163536, 114209 |
| 41508 | 167766817 | 167767557 | 173905 |
| 41509 | 167812219 | 167814771 | 173905 |
| 41510 | 167835455 | 167836234 | |
| 41511 | 167904237 | 167905833 | |
| 41512 | 167967130 | 167968079 | 206120 |
| 41513 | 168234701 | 168236176 | 206120 |
| 41514 | 168744338 | 168745352 | |
| 41515 | 169480463 | 169481360 | |
| 41516 | 169481789 | 169483646 | 270141 |
| 41517 | 169486947 | 169488151 | 269889, 184378 |
| 41518 | 169489326 | 169492121 | 085274, 269889 |
| 41519 | 169587335 | 169588237 | 114248 |
| 41520 | 169631973 | 169632614 | 187033 |
| 41521 | 169683840 | 169685885 | 008952, 239219 |
| 41522 | 169697140 | 169698376 | 008952, 240373 |
| 41523 | 169716456 | 169717461 | |
| 41524 | 169755327 | 169756245 | 173890 |
| 41525 | 169898158 | 169900067 | 173889 |
| 41526 | 169939404 | 169941133 | 163558 |
| 41527 | 169975961 | 169977181 | 163558 |
| 41528 | 170027858 | 170028885 | |
| 41529 | 170047977 | 170049768 | |
| 41530 | 170073398 | 170077536 | 136603 |
| 41531 | 170183135 | 170184537 | 013293, 013297 |
| 41532 | 170211198 | 170212426 | 013293, 013297 |
| 41533 | 170458762 | 170460353 | 239739, 013297 |
| 41534 | 170462342 | 170464489 | 013297 |
| 41535 | 170468691 | 170470599 | 013297 |
| 41536 | 170477219 | 170478977 | 013297 |
| 41537 | 170482513 | 170485522 | 013297 |
| 41538 | 170498241 | 170499348 | 013297 |
| 41539 | 170529020 | 170530244 | 013297 |
| 41540 | 170586961 | 170588474 | 163584 |
| 41541 | 170715516 | 170720768 | 163581 |
| 41542 | 170731467 | 170732444 | 163581 |
| 41543 | 170736013 | 170736772 | 163581 |
| 41544 | 170740100 | 170748119 | 163581 |
| 41545 | 170761970 | 170764156 | |
| 41546 | 170806467 | 170807843 | 154310 |
| 41547 | 170810068 | 170811053 | 154310 |
| 41548 | 170888636 | 170889503 | 154310 |
| 41549 | 171004336 | 171004804 | 154310 |
| 41550 | 171051770 | 171052769 | 154310 |
| 41551 | 171071080 | 171075172 | 154310 |
| 41552 | 171076401 | 171077411 | 154310 |
| 41553 | 171108239 | 171108814 | 154310 |
| 41554 | 171135475 | 171136178 | 154310 |
| 41555 | 171176932 | 171179153 | 154310 |
| 41556 | 171358040 | 171358717 | 075651 |
| 41557 | 171389360 | 171390291 | 075651 |
| 41558 | 171393909 | 171395119 | 075651 |
| 41559 | 171398552 | 171399384 | 075651 |
| 41560 | 171517151 | 171517902 | 183657, 075651 |
| 41561 | 171519528 | 171521116 | 183657, 075651 |
| 41562 | 171526768 | 171529700 | 183657, 075651 |
| 41563 | 171542939 | 171543710 | |
| 41564 | 171555878 | 171556999 | |
| 41565 | 171596564 | 171597037 | 234717, 186329 |
| 41566 | 171600911 | 171602438 | 234717, 186329 |
| 41567 | 171677315 | 171677890 | |
| 41568 | 171683927 | 171686665 | |
| 41569 | 171689744 | 171690126 | |
| 41570 | 171690751 | 171691356 | |
| 41571 | 171752832 | 171753523 | |
| 41572 | 171756966 | 171760117 | 075420 |
| 41573 | 171779372 | 171780095 | 075420 |
| 41574 | 171799511 | 171801130 | 075420 |
| 41575 | 171814698 | 171815346 | 075420 |
| 41576 | 171818634 | 171819408 | 075420 |
| 41577 | 171845633 | 171850321 | 075420 |
| 41578 | 171851400 | 171854735 | 075420 |
| 41579 | 171855326 | 171861272 | 075420 |
| 41580 | 171873061 | 171874744 | 075420 |
| 41581 | 171887045 | 171887615 | 075420 |
| 41582 | 171897143 | 171898843 | 075420 |
| 41583 | 171918834 | 171920242 | 075420 |
| 41584 | 171920324 | 171922209 | 075420 |
| 41585 | 171927339 | 171928261 | 075420 |
| 41586 | 171973494 | 171974481 | 075420 |
| 41587 | 171978718 | 171979403 | 075420 |
| 41588 | 171997995 | 171998789 | 075420 |
| 41589 | 172022489 | 172025014 | 075420 |
| 41590 | 172041573 | 172043199 | 075420 |
| 41591 | 172050149 | 172050759 | 075420 |
| 41592 | 172065637 | 172066725 | 075420 |
| 41593 | 172194198 | 172195536 | |
| 41594 | 172233979 | 172235858 | 121858 |
| 41595 | 172240841 | 172241638 | 121858 |
| 41596 | 172258773 | 172260242 | |
| 41597 | 172266656 | 172267358 | |
| 41598 | 172271113 | 172272127 | |
| 41599 | 172275065 | 172276493 | |
| 41600 | 172277859 | 172278432 | |
| 41601 | 172279557 | 172281802 | 223387 |
| 41602 | 172303992 | 172304696 | 223387 |
| 41603 | 172312076 | 172314650 | 223387 |
| 41604 | 172318615 | 172320145 | |
| 41605 | 172408668 | 172410383 | 144959 |
| 41606 | 172427742 | 172430342 | 144959 |
| 41607 | 172466258 | 172469368 | 114346 |
| 41608 | 172574842 | 172575574 | |
| 41609 | 174126773 | 174128170 | |
| 41610 | 174157594 | 174159779 | 177694 |
| 41611 | 174249123 | 174249979 | 177694 |
| 41612 | 174527984 | 174528528 | 177694 |
| 41613 | 174576999 | 174578148 | 177694 |
| 41614 | 174809342 | 174809843 | 230292, 177694 |
| 41615 | 174828683 | 174829434 | 230292, 177694 |
| 41616 | 174896627 | 174897668 | 177694 |
| 41617 | 175024833 | 175026478 | 177694 |
| 41618 | 175056503 | 175057602 | 177694 |
| 41619 | 175074529 | 175075422 | 177694 |
| 41620 | 175148724 | 175150026 | 177694 |
| 41621 | 175243929 | 175245137 | 177694 |
| 41622 | 175650278 | 175650836 | |
| 41623 | 176144068 | 176144569 | |
| 41624 | 176590132 | 176591055 | |
| 41625 | 176644843 | 176645780 | |
| 41626 | 176671071 | 176672022 | |
| 41627 | 176913077 | 176917157 | 177565 |
| 41628 | 176928680 | 176930608 | |
| 41629 | 177003308 | 177004620 | |
| 41630 | 177017703 | 177018589 | 203645 |
| 41631 | 177055393 | 177057008 | 226782 |
| 41632 | 177062859 | 177064492 | 226782 |
| 41633 | 177074684 | 177078619 | |
| 41634 | 177161160 | 177161787 | 228221 |
| 41635 | 177166974 | 177168197 | 228221 |
| 41636 | 177169989 | 177170619 | 228221 |
| 41637 | 177195391 | 177197767 | 228221 |
| 41638 | 177217343 | 177218430 | 228221 |
| 41639 | 177241473 | 177242417 | 228221 |
| 41640 | 177291883 | 177292700 | 228221 |
| 41641 | 177314647 | 177316231 | 228221 |
| 41642 | 177324594 | 177325445 | 228221 |
| 41643 | 177332658 | 177333390 | 228221 |
| 41644 | 177336853 | 177337384 | 228221 |
| 41645 | 177527491 | 177529036 | |
| 41646 | 177530787 | 177532142 | |
| 41647 | 177533742 | 177536000 | 231574 |
| 41648 | 177541440 | 177543236 | 231574 |
| 41649 | 177543905 | 177549490 | 231574 |
| 41650 | 177622767 | 177623322 | |
| 41651 | 177623829 | 177624719 | |
| 41652 | 177690943 | 177691892 | |
| 41653 | 178085335 | 178086150 | 223930, 197584 |
| 41654 | 178786715 | 178790450 | 172667 |
| 41655 | 178864849 | 178867749 | 121879, 229102 |
| 41656 | 178968721 | 178969440 | 171121 |
| 41657 | 178978820 | 178979434 | 240429, 171121 |
| 41658 | 179039346 | 179042177 | 121864 |
| 41659 | 179064316 | 179066486 | 171109 |
| 41660 | 179280250 | 179281765 | 136518 |
| 41661 | 179321458 | 179323460 | 136521, 136522 |
| 41662 | 179361589 | 179362750 | |
| 41663 | 179370252 | 179372432 | 058056 |
| 41664 | 179450486 | 179453394 | 058056 |
| 41665 | 179457611 | 179458186 | 058056 |
| 41666 | 179529336 | 179530214 | 114757 |
| 41667 | 180319178 | 180320803 | 145075, 163728, 239774 |
| 41668 | 180629754 | 180631735 | 114416 |
| 41669 | 180706736 | 180708057 | 242808, 205981 |
| 41670 | 181444583 | 181446682 | 242808 |
| 41671 | 181500027 | 181500822 | 242808 |
| 41672 | 181930069 | 181930958 | |
| 41673 | 181959870 | 181960623 | |
| 41674 | 181962171 | 181962929 | |
| 41675 | 182323435 | 182325176 | |
| 41676 | 182399994 | 182401827 | |
| 41677 | 182428828 | 182430062 | |
| 41678 | 182448071 | 182448651 | |
| 41679 | 182478358 | 182479115 | |
| 41680 | 182510343 | 182513075 | 058063, 240063 |
| 41681 | 182607097 | 182608344 | 058063 |
| 41682 | 182612938 | 182614822 | 058063 |
| 41683 | 182639300 | 182639931 | 058063, 273261 |
| 41684 | 182688373 | 182689025 | 043093 |
| 41685 | 182697160 | 182699368 | 043093 |
| 41686 | 182756540 | 182757273 | 078070 |
| 41687 | 182816079 | 182817799 | 078070 |
| 41688 | 182825564 | 182826435 | 078070 |
| 41689 | 182880066 | 182880792 | 078081 |
| 41690 | 182929156 | 182929892 | 053524 |
| 41691 | 182970789 | 182972930 | 176597, 053524 |
| 41692 | 182982849 | 182984724 | 176597, 053524 |
| 41693 | 182995805 | 182997022 | 176597, 053524 |
| 41694 | 183002327 | 183005885 | 176597, 053524 |
| 41695 | 183009356 | 183009981 | 176597, 053524 |
| 41696 | 183012906 | 183013438 | 176597, 053524 |
| 41697 | 183024166 | 183026157 | 053524 |
| 41698 | 183087399 | 183090074 | 053524 |
| 41699 | 183165061 | 183166180 | 240024 |
| 41700 | 183185971 | 183187707 | |
| 41701 | 183200916 | 183201911 | |
| 41702 | 183270382 | 183271194 | 172578 |
| 41703 | 183344551 | 183345061 | |
| 41704 | 183352683 | 183355522 | 114796 |
| 41705 | 183371350 | 183372012 | 114796 |
| 41706 | 183415068 | 183416614 | 163872 |
| 41707 | 183463170 | 183463769 | 163872 |
| 41708 | 183601580 | 183603170 | 234371, 175193 |
| 41709 | 183702757 | 183703532 | 114770 |
| 41710 | 183705816 | 183706485 | 114770 |
| 41711 | 183735025 | 183736455 | 114770 |
| 41712 | 183851337 | 183852301 | 272721, 273181 |
| 41713 | 183852431 | 183854234 | 145191, 272721 |
| 41714 | 183872542 | 183875137 | 161202, 145191 |
| 41715 | 183892224 | 183893541 | 161203, 145191 |
| 41716 | 183902809 | 183905145 | 161204, 145191 |
| 41717 | 183959407 | 183960105 | 214160, 145198, 145191 |
| 41718 | 183965784 | 183968409 | 145194, 214160, 145191 |
| 41719 | 184016512 | 184018462 | 145191, 175166 |
| 41720 | 184031964 | 184034443 | 114867, 145191 |
| 41721 | 184037493 | 184038784 | 145191, 114867 |
| 41722 | 184049468 | 184055114 | 175182, 114867, 145191 |
| 41723 | 184069592 | 184070641 | 145191, 114859 |
| 41724 | 184078857 | 184081902 | 163882, 114859, 145191 |
| 41725 | 184094892 | 184098672 | 090539, 090534, 145191 |
| 41726 | 184298756 | 184300216 | 182580, 145191 |
| 41727 | 184363080 | 184363612 | 145191 |
| 41728 | 184428720 | 184430243 | 177383 |
| 41729 | 184529646 | 184530681 | 156931 |
| 41730 | 184933646 | 184934721 | 113790 |
| 41731 | 184943148 | 184944356 | 113790 |
| 41732 | 184968010 | 184972316 | 264614, 113790 |
| 41733 | 184999957 | 185001592 | 073803 |
| 41734 | 185033932 | 185036921 | 073803 |
| 41735 | 185045853 | 185048975 | 073803 |
| 41736 | 185049602 | 185051372 | 073803 |
| 41737 | 185053516 | 185054130 | 073803 |
| 41738 | 185054233 | 185056015 | 073803 |
| 41739 | 185079230 | 185080776 | 073803 |
| 41740 | 185138478 | 185138979 | 073803 |
| 41741 | 185140984 | 185142078 | 073803 |
| 41742 | 185206886 | 185208036 | 073803, 163900 |
| 41743 | 185215837 | 185217213 | 163900 |
| 41744 | 185255110 | 185255576 | 163898 |
| 41745 | 185276012 | 185276694 | |
| 41746 | 185303438 | 185304840 | 163904 |
| 41747 | 185375693 | 185377377 | 073792 |
| 41748 | 185514270 | 185514900 | 073792 |
| 41749 | 185617985 | 185618387 | |
| 41750 | 185653772 | 185656234 | 136527 |
| 41751 | 185826611 | 185828251 | 244405, 058866 |
| 41752 | 185959375 | 185960806 | 058866 |
| 41753 | 186130741 | 186132788 | |
| 41754 | 186258046 | 186258948 | 213139 |
| 41755 | 186284072 | 186286207 | 090520, 113838 |
| 41756 | 186287875 | 186289739 | 113838, 090520 |
| 41757 | 186306178 | 186309027 | 090520 |
| 41758 | 186311258 | 186313502 | 090520 |
| 41759 | 186315644 | 186319005 | |
| 41760 | 186322241 | 186325215 | |
| 41761 | 186330260 | 186334332 | 145192 |
| 41762 | 186334509 | 186335406 | 145192 |
| 41763 | 186351187 | 186351715 | |
| 41764 | 186352001 | 186352507 | |
| 41765 | 186357802 | 186362605 | 236524, 090512 |
| 41766 | 186383288 | 186384649 | 236524, 113905 |
| 41767 | 186434470 | 186435502 | 197099, 113889 |
| 41768 | 186436004 | 186440696 | 197099, 113889 |
| 41769 | 186459866 | 186464636 | 113889, 197099 |
| 41770 | 186470526 | 186472666 | 197099 |
| 41771 | 186478148 | 186478992 | 226652, 197099 |
| 41772 | 186489930 | 186491345 | 230951 |
| 41773 | 186500030 | 186503106 | 156976, 238942, 263826 |
| 41774 | 186503238 | 186503649 | 156976 |
| 41775 | 186507609 | 186509413 | 163918, 156976 |
| 41776 | 186523508 | 186525272 | 163918 |
| 41777 | 186648235 | 186650182 | 073849 |
| 41778 | 186665646 | 186666155 | 073849 |
| 41779 | 186692450 | 186693135 | 073849 |
| 41780 | 186694520 | 186695821 | 073849 |
| 41781 | 186708236 | 186710010 | 073849 |
| 41782 | 186710266 | 186711117 | 073849 |
| 41783 | 186756200 | 186763054 | 073849 |
| 41784 | 186780216 | 186782796 | 073849 |
| 41785 | 186819350 | 186820339 | |
| 41786 | 186847713 | 186848958 | 163923 |
| 41787 | 186857041 | 186857798 | 163923 |
| 41788 | 186891403 | 186892488 | 163923 |
| 41789 | 186904476 | 186905044 | |
| 41790 | 186981640 | 186983206 | 127241 |
| 41791 | 187003219 | 187007868 | 127241 |
| 41792 | 187008611 | 187010491 | 127241 |
| 41793 | 187036345 | 187038093 | |
| 41794 | 187453158 | 187464888 | 223401, 113916 |
| 41795 | 187467557 | 187468913 | |
| 41796 | 187512947 | 187513854 | |
| 41797 | 187517398 | 187518253 | 224049 |
| 41798 | 187526785 | 187527445 | |
| 41799 | 187557246 | 187557644 | |
| 41800 | 187570473 | 187571568 | |
| 41801 | 187596546 | 187597327 | |
| 41802 | 187664838 | 187665336 | |
| 41803 | 187671894 | 187674070 | |
| 41804 | 187681496 | 187683065 | 224187 |
| 41805 | 187685845 | 187687859 | 224187 |
| 41806 | 187720208 | 187721543 | 236412, 225058 |
| 41807 | 187753882 | 187755520 | |
| 41808 | 187778855 | 187779504 | |
| 41809 | 187782273 | 187782962 | |
| 41810 | 187870295 | 187873683 | 145012, 270959 |
| 41811 | 187875432 | 187876520 | 145012 |
| 41812 | 187881904 | 187882632 | 145012 |
| 41813 | 187888319 | 187889385 | 145012 |
| 41814 | 187923767 | 187924677 | 145012 |
| 41815 | 187929599 | 187931178 | 145012 |
| 41816 | 187937400 | 187940323 | 145012 |
| 41817 | 187951037 | 187952114 | 145012 |
| 41818 | 187980089 | 187985314 | 145012 |
| 41819 | 187987891 | 187988968 | 145012 |
| 41820 | 188003053 | 188004538 | 145012 |
| 41821 | 188008070 | 188009144 | 145012 |
| 41822 | 188011758 | 188016474 | 145012 |
| 41823 | 188034090 | 188034677 | 145012 |
| 41824 | 188065902 | 188068233 | 145012 |
| 41825 | 188080188 | 188080671 | 145012 |
| 41826 | 188114589 | 188116325 | 145012 |
| 41827 | 188186283 | 188187595 | 145012 |
| 41828 | 188236159 | 188237216 | 145012 |
| 41829 | 188296829 | 188297230 | 145012 |
| 41830 | 188300683 | 188303162 | 145012 |
| 41831 | 188341864 | 188342693 | 145012 |
| 41832 | 188400082 | 188401776 | 145012 |
| 41833 | 188401795 | 188402554 | 145012 |
| 41834 | 188406428 | 188407482 | 207651, 145012 |
| 41835 | 188429951 | 188430751 | 145012 |
| 41836 | 188437701 | 188439666 | 145012 |
| 41837 | 188457652 | 188458412 | 145012 |
| 41838 | 188460581 | 188462707 | 145012 |
| 41839 | 188463475 | 188465146 | 145012 |
| 41840 | 188490564 | 188491229 | 145012 |
| 41841 | 188498123 | 188498932 | 145012 |
| 41842 | 188598068 | 188600847 | 145012 |
| 41843 | 188632299 | 188633464 | |
| 41844 | 188650163 | 188650815 | |
| 41845 | 188662480 | 188670031 | 188001, 234076 |
| 41846 | 188816411 | 188817638 | 188001 |
| 41847 | 188840933 | 188841783 | 188001 |
| 41848 | 189042323 | 189043180 | 188001 |
| 41849 | 189073000 | 189073763 | |
| 41850 | 189339655 | 189340230 | |
| 41851 | 189347290 | 189349111 | |
| 41852 | 189408187 | 189409084 | 073282 |
| 41853 | 189427372 | 189431342 | 073282 |
| 41854 | 189566457 | 189566995 | 073282 |
| 41855 | 189577943 | 189578810 | 073282 |
| 41856 | 189580504 | 189581197 | 073282 |
| 41857 | 189616305 | 189616746 | |
| 41858 | 189699307 | 189699875 | 090530 |
| 41859 | 189729559 | 189731187 | 090530 |
| 41860 | 189793557 | 189795730 | 090530 |
| 41861 | 189934869 | 189935329 | |
| 41862 | 189946685 | 189951336 | |
| 41863 | 189998854 | 189999644 | |
| 41864 | 190016364 | 190018922 | |
| 41865 | 190019324 | 190027834 | 163347 |
| 41866 | 190034597 | 190041873 | 113946, 163347 |
| 41867 | 190047879 | 190049643 | 113946 |
| 41868 | 190053692 | 190054958 | 113946 |
| 41869 | 190061953 | 190065522 | 113946 |
| 41870 | 190083331 | 190084863 | 113946 |
| 41871 | 190095640 | 190096894 | 113946 |
| 41872 | 190098627 | 190101555 | 113946 |
| 41873 | 190222453 | 190223649 | |
| 41874 | 190230972 | 190233126 | 196083 |
| 41875 | 190244820 | 190245614 | 196083 |
| 41876 | 190265914 | 190266784 | 196083 |
| 41877 | 190283465 | 190286229 | 196083 |
| 41878 | 190302345 | 190305377 | 196083 |
| 41879 | 190308412 | 190311258 | 196083 |
| 41880 | 190332270 | 190333209 | 196083 |
| 41881 | 190359019 | 190359878 | 196083 |
| 41882 | 190418563 | 190419667 | |
| 41883 | 190525400 | 190526005 | |
| 41884 | 190529421 | 190530853 | |
| 41885 | 190543084 | 190544129 | |
| 41886 | 190579963 | 190581564 | 205835 |
| 41887 | 190583054 | 190583928 | 205835 |
| 41888 | 190783040 | 190785465 | |
| 41889 | 190978282 | 190979352 | 188729 |
| 41890 | 191037920 | 191038544 | 188958 |
| 41891 | 191046337 | 191050629 | 152492, 188958 |
| 41892 | 191055367 | 191056794 | 152492 |
| 41893 | 191073766 | 191075571 | 152492 |
| 41894 | 191147452 | 191150382 | |
| 41895 | 191445060 | 191445565 | |
| 41896 | 191458720 | 191460651 | |
| 41897 | 191493216 | 191494539 | |
| 41898 | 191512923 | 191513398 | |
| 41899 | 192001544 | 192003423 | 114279 |
| 41900 | 192293541 | 192294317 | 114279 |
| 41901 | 192569554 | 192570247 | 180611 |
| 41902 | 192646413 | 192647239 | |
| 41903 | 193310192 | 193312168 | 198836, 236297, 127249 |
| 41904 | 193380271 | 193381477 | 198836 |
| 41905 | 193450001 | 193451186 | |
| 41906 | 193460493 | 193463567 | |
| 41907 | 193551792 | 193553042 | |
| 41908 | 193589837 | 193590572 | |
| 41909 | 193720620 | 193723380 | 230102, 214146, 238043 |
| 41910 | 193749258 | 193750599 | 230102 |
| 41911 | 193752067 | 193752985 | 230102 |
| 41912 | 193764913 | 193765702 | 230102 |
| 41913 | 193775390 | 193777189 | 230102 |
| 41914 | 193787474 | 193789754 | 230102 |
| 41915 | 193793093 | 193794743 | |
| 41916 | 193806990 | 193808278 | |
| 41917 | 193814600 | 193816239 | 229964 |
| 41918 | 193818469 | 193820059 | 229964 |
| 41919 | 193829881 | 193831369 | |
| 41920 | 193839597 | 193840316 | |
| 41921 | 193847262 | 193848109 | |
| 41922 | 193848445 | 193849671 | 232874 |
| 41923 | 193850685 | 193856384 | 114315 |
| 41924 | 193856686 | 193860814 | |
| 41925 | 193867956 | 193868760 | |
| 41926 | 193921784 | 193922211 | 225742 |
| 41927 | 193964852 | 193966416 | 238097, 225742 |
| 41928 | 193967808 | 193968748 | 225742, 238097 |
| 41929 | 193987041 | 193989811 | |
| 41930 | 193994347 | 193996077 | 228271 |
| 41931 | 194014064 | 194015690 | 214145 |
| 41932 | 194032623 | 194033976 | |
| 41933 | 194051203 | 194052388 | |
| 41934 | 194052911 | 194055038 | |
| 41935 | 194067302 | 194067941 | 178772 |
| 41936 | 194070928 | 194072606 | 178772 |
| 41937 | 194167431 | 194168444 | 133657 |
| 41938 | 194205666 | 194210779 | 233058, 133657 |
| 41939 | 194214354 | 194216321 | 133657, 233058 |
| 41940 | 194216774 | 194217574 | 244675, 133657, 233058 |
| 41941 | 194238864 | 194240079 | 233058 |
| 41942 | 194241964 | 194244442 | |
| 41943 | 194256959 | 194259174 | |
| 41944 | 194292061 | 194297414 | |
| 41945 | 194303925 | 194305944 | 231770 |
| 41946 | 194341761 | 194342342 | 145014 |
| 41947 | 194353401 | 194354696 | 229334, 145014 |
| 41948 | 194391987 | 194394109 | 041802 |
| 41949 | 194403033 | 194403978 | |
| 41950 | 194489649 | 194490267 | 237222 |
| 41951 | 194538127 | 194538724 | |
| 41952 | 194590646 | 194593754 | |
| 41953 | 194696612 | 194697129 | |
| 41954 | 194752038 | 194752883 | |
| 41955 | 194754033 | 194755177 | |
| 41956 | 194825918 | 194827708 | 173950 |
| 41957 | 194907995 | 194909392 | 173950 |
| 41958 | 194979543 | 194981370 | 173950 |
| 41959 | 194991196 | 194992953 | 173950 |
| 41960 | 195162164 | 195163909 | 114331 |
| 41961 | 195269308 | 195270537 | 184203 |
| 41962 | 195442007 | 195444638 | 242086 |
| 41963 | 195542219 | 195542652 | |
| 41964 | 195573771 | 195575144 | 223783 |
| 41965 | 195577586 | 195578282 | 223783 |
| 41966 | 195579110 | 195579699 | 223783 |
| 41967 | 195616354 | 195617231 | 061938 |
| 41968 | 195636211 | 195636928 | 224614, 061938 |
| 41969 | 195641234 | 195642357 | |
| 41970 | 195807359 | 195809864 | 072274 |
| 41971 | 195862674 | 195863752 | |
| 41972 | 195941669 | 195942563 | 163959, 161217 |
| 41973 | 195943066 | 195944235 | 163959, 161217 |
| 41974 | 196006077 | 196007385 | 272741, 161217 |
| 41975 | 196013637 | 196015601 | 161217, 272741 |
| 41976 | 196044254 | 196045792 | 235897, 272741, 213123, |
| | | | 273331 |
| 41977 | 196158146 | 196159834 | 225822, 163960 |
| 41978 | 196227666 | 196231109 | 163961 |
| 41979 | 196294541 | 196296510 | 174013, 185798 |
| 41980 | 196358573 | 196360261 | 232065 |
| 41981 | 196438665 | 196440044 | 174007, 163964 |
| 41982 | 196466323 | 196469025 | 180370 |
| 41983 | 196500498 | 196502226 | 180370 |
| 41984 | 196519327 | 196520725 | 180370 |
| 41985 | 196594146 | 196595712 | 119231 |
| 41986 | 196668111 | 196670493 | 270170, 225578, 114503 |
| 41987 | 196695306 | 196697175 | 119227 |
| 41988 | 196729257 | 196731084 | 228109, 163975 |
| 41989 | 196755855 | 196759541 | 163975 |
| 41990 | 196997302 | 196997927 | 075711 |
| 41991 | 197023864 | 197026983 | 227375, 075711 |
| 41992 | 197033226 | 197033735 | |
| 41993 | 197036459 | 197037194 | |
| 41994 | 197079145 | 197079697 | |
| 41995 | 197082388 | 197083059 | |
| 41996 | 197182458 | 197184372 | 229912, 235126 |
| 41997 | 197192794 | 197194046 | 237167 |
| 41998 | 197194845 | 197196727 | |
| 41999 | 197198178 | 197199560 | |
| 42000 | 197205145 | 197208180 | |
| 42001 | 197214646 | 197215763 | |
| 42002 | 197243313 | 197244498 | 161267 |
| 42003 | 197246971 | 197249245 | 161267 |
| 42004 | 197286377 | 197287007 | 161267 |
| 42005 | 197299754 | 197300794 | 161267 |
| 42006 | 197316830 | 197317913 | 214135 |
| 42007 | 197324169 | 197324891 | 214135 |
| 42008 | 197330765 | 197331537 | 214135 |
| 42009 | 197444926 | 197446451 | 145016 |
| 42010 | 197462818 | 197464731 | 122068, 145016 |
| 42011 | 197476311 | 197478464 | 145016, 122068 |
| 42012 | 197517521 | 197519395 | 186001 |
| 42013 | 197521978 | 197522557 | 186001 |
| 42014 | 197546944 | 197548788 | 186001 |
| 42015 | 197574616 | 197575462 | 186001 |
| 42016 | 197676391 | 197678298 | 182899, 114473 |
| 42017 | 197686076 | 197688410 | 185621, 114473 |
| 42018 | 197807337 | 197808161 | 226435 |

The locations of ODSCs on Chromosome 4 are shown in Table 46.

TABLE 46
ODSC Locations on Chromosome 4

| SEQ ID NO. | Start | End | Overlapping Gene(s) |
|---|---|---|---|
| 42019 | 52932 | 54026 | 197701 |
| 42020 | 124147 | 125159 | 250312, 256143 |
| 42021 | 330409 | 332349 | 131127, 248564, 251535 |
| 42022 | 492167 | 494088 | 174227, 182903 |
| 42023 | 667076 | 669403 | 215375, 169020 |
| 42024 | 680594 | 682475 | 169026 |
| 42025 | 698953 | 700552 | 185619 |
| 42026 | 774238 | 776809 | 249592 |
| 42027 | 782425 | 783348 | 168993 |
| 42028 | 924828 | 927091 | 127419, 178950 |
| 42029 | 978048 | 979536 | 145217, 145214 |
| 42030 | 980058 | 982297 | 127415, 145214, 145217 |
| 42031 | 985178 | 988341 | 145217, 127415 |
| 42032 | 1004462 | 1005463 | 127418 |
| 42033 | 1005477 | 1006620 | 127418 |
| 42034 | 1037205 | 1038781 | |
| 42035 | 1066486 | 1067025 | 178222 |
| 42036 | 1161003 | 1161620 | 273179, 159674 |
| 42037 | 1165448 | 1168170 | 159674 |
| 42038 | 1170641 | 1171832 | 159674 |
| 42039 | 1185273 | 1186907 | 159674 |
| 42040 | 1197993 | 1199462 | 159674 |
| 42041 | 1234944 | 1235902 | 159692 |
| 42042 | 1240418 | 1244389 | 196810, 159692 |
| 42043 | 1283160 | 1284956 | 090316 |
| 42044 | 1330243 | 1332616 | 090316 |
| 42045 | 1339631 | 1342484 | 163945 |
| 42046 | 1580405 | 1582247 | 244459 |
| 42047 | 1685129 | 1686579 | 207009, 174137 |
| 42048 | 1712723 | 1715367 | 270195, 163950 |
| 42049 | 1719693 | 1720943 | 168936 |
| 42050 | 1721548 | 1723507 | 013810, 168936 |
| 42051 | 1768768 | 1769840 | |
| 42052 | 1794829 | 1796190 | 068078 |
| 42053 | 1796494 | 1798492 | 068078 |
| 42054 | 1817863 | 1818556 | 168924 |
| 42055 | 1856645 | 1858927 | 168924 |
| 42056 | 1871918 | 1874341 | 109685 |
| 42057 | 1880024 | 1880740 | 109685 |
| 42058 | 1881765 | 1882588 | 109685 |
| 42059 | 2009589 | 2012559 | 185049 |
| 42060 | 2085494 | 2085918 | 130997 |
| 42061 | 2242899 | 2244308 | 130997, 214367 |
| 42062 | 2262238 | 2264975 | 123933 |
| 42063 | 2335044 | 2335533 | 159733 |
| 42064 | 2364910 | 2365592 | 159733 |
| 42065 | 2469609 | 2472316 | 063978 |
| 42066 | 2536748 | 2539653 | 063978 |
| 42067 | 2585163 | 2585643 | 063978 |
| 42068 | 2699475 | 2700289 | 125386 |
| 42069 | 2746736 | 2749512 | 168884 |
| 42070 | 2756968 | 2758330 | 168884 |
| 42071 | 2792384 | 2797377 | 087266 |
| 42072 | 2804651 | 2806967 | 087266 |
| 42073 | 2811241 | 2811845 | 087266 |
| 42074 | 2844278 | 2846599 | 087274 |
| 42075 | 2859857 | 2860806 | 087274 |
| 42076 | 2871365 | 2872151 | 087274 |
| 42077 | 2911225 | 2911852 | 087274 |
| 42078 | 2935277 | 2939571 | 249673, 109736 |
| 42079 | 2948395 | 2950276 | 249673, 087269 |
| 42080 | 2964115 | 2966409 | 125388, 087269 |
| 42081 | 3042746 | 3044068 | |
| 42082 | 3075102 | 3075966 | 251075 |
| 42083 | 3076089 | 3077533 | 197386, 251075 |
| 42084 | 3108201 | 3108742 | 197386 |
| 42085 | 3180551 | 3182047 | 197386 |
| 42086 | 3204645 | 3205654 | 197386 |
| 42087 | 3209187 | 3209798 | 197386 |
| 42088 | 3293301 | 3295768 | 159788 |
| 42089 | 3355490 | 3357612 | 159788 |
| 42090 | 3385847 | 3388827 | 159788 |
| 42091 | 3409572 | 3410867 | 159788 |
| 42092 | 3437447 | 3439926 | 159788 |
| 42093 | 3443154 | 3445828 | 109758 |
| 42094 | 3463109 | 3465650 | 175920 |
| 42095 | 3533183 | 3534851 | 163956 |
| 42096 | 3786173 | 3786981 | |
| 42097 | 3787856 | 3788471 | |
| 42098 | 3820629 | 3821512 | |
| 42099 | 3828052 | 3828632 | |
| 42100 | 3828865 | 3830257 | |
| 42101 | 4249018 | 4251086 | 132406 |
| 42102 | 4261081 | 4262445 | |
| 42103 | 4291832 | 4292925 | 168826, 145220 |
| 42104 | 4432203 | 4433274 | 168818 |
| 42105 | 4498689 | 4499226 | 168818 |
| 42106 | 4542703 | 4544766 | 247708, 168818 |
| 42107 | 4576533 | 4577693 | 247708 |
| 42108 | 5039335 | 5040379 | |
| 42109 | 5748525 | 5749536 | 072840 |
| 42110 | 5759556 | 5761657 | 072840, 072832 |
| 42111 | 6073723 | 6074483 | 152969 |
| 42112 | 6271140 | 6272956 | 109501 |
| 42113 | 6576381 | 6577383 | 013288 |
| 42114 | 6577994 | 6578888 | 013288 |
| 42115 | 6641560 | 6643768 | 179010 |
| 42116 | 6675007 | 6676675 | 170846, 251580 |
| 42117 | 6690765 | 6691707 | 246526 |
| 42118 | 6697234 | 6697971 | 163993 |
| 42119 | 6710228 | 6712515 | 178988 |
| 42120 | 6717436 | 6719196 | 186222 |
| 42121 | 6733700 | 6736138 | |
| 42122 | 6748647 | 6750048 | |
| 42123 | 6750662 | 6753199 | |
| 42124 | 6753215 | 6754022 | |
| 42125 | 6754272 | 6755650 | |
| 42126 | 6756234 | 6757459 | |
| 42127 | 6775832 | 6776272 | |
| 42128 | 6783543 | 6785724 | 170871 |
| 42129 | 6841755 | 6842303 | 170871 |
| 42130 | 6887170 | 6888399 | |
| 42131 | 6893070 | 6896946 | |
| 42132 | 6910041 | 6913586 | 132405 |
| 42133 | 6918693 | 6919909 | 132405 |
| 42134 | 6928550 | 6929230 | 132405 |
| 42135 | 6949289 | 6949871 | 132405 |
| 42136 | 6987636 | 6990243 | 132405 |
| 42137 | 7009588 | 7010124 | 132405 |
| 42138 | 7016292 | 7017163 | 132405 |
| 42139 | 7030744 | 7031359 | 132405 |
| 42140 | 7036264 | 7037055 | 245748 |
| 42141 | 7038021 | 7038371 | 245748 |
| 42142 | 7044373 | 7046444 | 173013, 245748, 173011 |
| 42143 | 7068526 | 7071119 | 109519 |
| 42144 | 7104762 | 7106183 | 245468 |
| 42145 | 7126786 | 7127404 | |
| 42146 | 7207751 | 7209444 | 184985 |
| 42147 | 7211355 | 7211999 | 184985 |
| 42148 | 7219036 | 7220845 | 184985 |
| 42149 | 7516929 | 7517865 | 184985 |
| 42150 | 7548340 | 7549839 | 184985 |
| 42151 | 7559113 | 7560795 | 184985 |
| 42152 | 7561397 | 7562636 | 184985 |
| 42153 | 7565410 | 7567270 | 184985 |
| 42154 | 7632217 | 7632969 | 184985 |
| 42155 | 7640628 | 7641154 | 184985 |
| 42156 | 7649754 | 7650868 | 184985 |
| 42157 | 7751327 | 7751880 | |
| 42158 | 7807134 | 7807941 | 196526 |
| 42159 | 8114723 | 8115808 | 163995 |
| 42160 | 8145905 | 8147766 | 163995 |
| 42161 | 8159981 | 8161158 | 163995 |
| 42162 | 8200603 | 8202427 | 125089 |
| 42163 | 8216735 | 8217202 | 125089 |
| 42164 | 8229319 | 8231334 | 125089 |
| 42165 | 8239653 | 8240972 | 125089 |
| 42166 | 8304661 | 8306280 | 170801 |
| 42167 | 8421028 | 8422237 | 087008 |
| 42168 | 8428924 | 8430478 | 087008 |
| 42169 | 8440902 | 8443601 | 087008, 155275 |
| 42170 | 8535615 | 8536429 | |
| 42171 | 8538257 | 8541647 | |
| 42172 | 10000892 | 10004586 | 109667 |
| 42173 | 10007678 | 10010255 | 250413, 109667 |
| 42174 | 10018459 | 10019370 | 109667 |
| 42175 | 10021264 | 10022002 | 109667 |
| 42176 | 10022215 | 10023667 | 109667 |
| 42177 | 10033688 | 10035279 | 109667 |
| 42178 | 10046902 | 10047560 | 109667 |
| 42179 | 10106987 | 10109140 | 071127 |
| 42180 | 10116858 | 10119783 | 223086, 071127 |
| 42181 | 10163692 | 10164744 | |
| 42182 | 10187342 | 10188225 | |
| 42183 | 10206134 | 10207384 | |
| 42184 | 12853753 | 12854905 | |
| 42185 | 12934974 | 12936106 | |
| 42186 | 12949957 | 12952744 | 250098 |
| 42187 | 13485249 | 13486586 | 157869 |
| 42188 | 13627957 | 13630452 | 266240, 038219 |
| 42189 | 13679540 | 13680931 | 250634 |
| 42190 | 13702464 | 13704874 | 250634 |
| 42191 | 13705438 | 13708671 | 250634 |
| 42192 | 13857767 | 13858655 | 250634 |
| 42193 | 13908263 | 13910030 | 250634 |
| 42194 | 13921405 | 13923274 | 250634 |
| 42195 | 13992344 | 13993348 | |
| 42196 | 14182425 | 14183408 | 249988 |
| 42197 | 14831366 | 14831878 | 248360 |
| 42198 | 14854749 | 14855854 | 248360 |
| 42199 | 14857513 | 14858614 | 248360 |
| 42200 | 14890365 | 14891109 | |
| 42201 | 14893756 | 14894763 | |
| 42202 | 14896446 | 14897132 | |
| 42203 | 14910094 | 14911173 | |
| 42204 | 14911767 | 14912888 | 247624 |
| 42205 | 14917671 | 14919682 | 247624 |
| 42206 | 14959032 | 14960638 | 247624 |
| 42207 | 15002040 | 15007737 | 249252, 137449, 247624 |
| 42208 | 15078675 | 15081345 | 249252 |
| 42209 | 15102604 | 15103258 | 249252 |
| 42210 | 15107507 | 15109030 | 249252 |
| 42211 | 15111073 | 15112299 | 249252 |
| 42212 | 15133117 | 15133599 | 249252 |
| 42213 | 15150700 | 15152473 | 249252 |
| 42214 | 15480503 | 15481772 | 048342 |
| 42215 | 15553392 | 15554080 | 048342 |
| 42216 | 15655830 | 15658438 | 118564 |
| 42217 | 15678580 | 15679805 | 118564 |
| 42218 | 15682657 | 15684423 | 237765, 118564 |
| 42219 | 15691740 | 15693635 | 237765 |
| 42220 | 15742283 | 15743025 | |
| 42221 | 15780196 | 15781687 | 004468 |
| 42222 | 15906885 | 15908744 | |
| 42223 | 15921748 | 15923921 | |
| 42224 | 16027674 | 16029762 | 007062 |
| 42225 | 16034676 | 16037604 | 007062 |
| 42226 | 16224495 | 16225235 | 169762 |
| 42227 | 16225915 | 16229761 | 263327, 169762 |
| 42228 | 16251071 | 16253355 | 263327 |
| 42229 | 16254212 | 16256438 | 263327 |
| 42230 | 16723095 | 16724101 | 169744 |
| 42231 | 16825758 | 16826717 | 169744 |
| 42232 | 16837176 | 16838547 | 169744 |
| 42233 | 16839819 | 16840607 | 169744 |
| 42234 | 17331681 | 17332365 | |
| 42235 | 17491211 | 17500524 | 151552 |
| 42236 | 17512306 | 17518324 | 249581, 151552 |
| 42237 | 17550557 | 17551688 | |
| 42238 | 17560234 | 17561642 | |
| 42239 | 17578385 | 17580106 | 002549 |
| 42240 | 17615164 | 17616979 | 118579, 249502 |
| 42241 | 17811628 | 17813049 | 109805, 163257 |
| 42242 | 18022325 | 18024662 | 178177 |
| 42243 | 18690969 | 18691476 | |
| 42244 | 20590698 | 20591575 | 145147 |
| 42245 | 20647769 | 20648481 | |
| 42246 | 20701583 | 20702396 | 163138 |
| 42247 | 22239582 | 22240699 | 250039 |
| 42248 | 22437190 | 22437771 | 152990 |
| 42249 | 22476597 | 22477000 | 152990 |
| 42250 | 22501779 | 22503457 | 152990 |
| 42251 | 22515444 | 22518499 | 152990 |
| 42252 | 22694185 | 22696193 | 249948 |
| 42253 | 22697445 | 22699230 | 249948 |
| 42254 | 22713636 | 22714873 | 249948 |
| 42255 | 22741479 | 22742074 | 249948 |
| 42256 | 23374783 | 23376785 | |
| 42257 | 23384463 | 23384955 | |
| 42258 | 23631590 | 23632265 | 250137 |
| 42259 | 23820909 | 23821502 | 109819 |
| 42260 | 23875753 | 23876705 | 109819 |
| 42261 | 23879164 | 23880025 | 109819 |
| 42262 | 23882668 | 23884006 | 109819 |
| 42263 | 23889465 | 23893673 | 109819 |
| 42264 | 23905619 | 23906246 | 109819 |
| 42265 | 23923597 | 23925188 | |
| 42266 | 23931710 | 23933876 | |
| 42267 | 23936385 | 23937085 | |
| 42268 | 23982982 | 23983384 | |
| 42269 | 24009172 | 24010147 | |
| 42270 | 24056888 | 24057948 | |
| 42271 | 24068786 | 24070496 | |
| 42272 | 24075151 | 24076326 | |
| 42273 | 24106270 | 24107884 | |
| 42274 | 24138981 | 24140340 | |
| 42275 | 24161970 | 24162690 | |
| 42276 | 24193736 | 24194467 | |
| 42277 | 24204048 | 24204685 | |
| 42278 | 24210335 | 24213739 | |
| 42279 | 24219678 | 24222051 | |
| 42280 | 24225290 | 24227136 | |
| 42281 | 24229559 | 24230965 | |
| 42282 | 24231315 | 24233552 | |
| 42283 | 24236061 | 24238706 | |
| 42284 | 24279956 | 24282672 | |
| 42285 | 24313858 | 24314682 | |
| 42286 | 24382342 | 24384571 | |
| 42287 | 24430296 | 24431096 | |
| 42288 | 24474228 | 24475107 | |
| 42289 | 24519568 | 24520643 | 109606 |
| 42290 | 24558542 | 24559042 | 109606 |
| 42291 | 24584982 | 24586955 | 109606 |
| 42292 | 24644286 | 24645257 | |
| 42293 | 24659672 | 24660344 | 249256 |
| 42294 | 24796500 | 24797180 | 109610 |
| 42295 | 24913767 | 24915188 | 181982 |
| 42296 | 25155063 | 25157386 | 109618 |
| 42297 | 25160624 | 25163644 | 038210, 109618 |
| 42298 | 25180217 | 25180916 | 038210 |
| 42299 | 25214314 | 25215993 | 038210 |
| 42300 | 25222954 | 25223419 | 038210 |
| 42301 | 25235017 | 25237566 | 038210 |
| 42302 | 25253727 | 25255865 | 038210 |
| 42303 | 25260771 | 25261239 | 038210 |
| 42304 | 25313925 | 25315259 | 168228 |
| 42305 | 25378129 | 25379659 | 053900 |
| 42306 | 25426098 | 25426912 | |
| 42307 | 25789139 | 25790305 | 091490 |
| 42308 | 25815188 | 25815782 | 091490 |
| 42309 | 25863485 | 25865458 | 091490, 250317 |
| 42310 | 25915556 | 25916893 | 250317 |
| 42311 | 26320399 | 26323492 | 168214 |
| 42312 | 26414270 | 26415036 | 168214 |
| 42313 | 26584768 | 26586426 | 109680 |
| 42314 | 26755124 | 26755697 | 109680 |
| 42315 | 26788971 | 26790749 | |
| 42316 | 26822837 | 26824754 | |
| 42317 | 26858291 | 26859978 | 109689 |
| 42318 | 26861148 | 26863869 | 240005, 109689 |
| 42319 | 26880142 | 26881140 | 109689 |
| 42320 | 27007534 | 27008269 | 109689 |
| 42321 | 27644739 | 27645103 | |
| 42322 | 28289088 | 28290371 | |
| 42323 | 28315473 | 28316445 | |
| 42324 | 29306180 | 29306683 | |
| 42325 | 32477325 | 32477757 | |
| 42326 | 32842067 | 32843420 | |
| 42327 | 32866368 | 32867001 | |
| 42328 | 33075148 | 33075943 | |
| 42329 | 33950397 | 33950963 | 250723, 250954 |
| 42330 | 34182226 | 34184389 | 248685 |
| 42331 | 34201870 | 34202687 | 248685 |
| 42332 | 34733855 | 34735510 | |
| 42333 | 36038674 | 36039222 | 047365 |
| 42334 | 36244512 | 36246937 | 247193, 047365 |
| 42335 | 36350043 | 36350823 | 251438 |
| 42336 | 36364511 | 36365084 | 251438 |
| 42337 | 37431799 | 37433455 | 174145 |
| 42338 | 37442552 | 37444366 | 174145 |
| 42339 | 37454478 | 37456666 | 154274 |
| 42340 | 37457045 | 37457958 | 154274 |
| 42341 | 37529538 | 37530504 | 154274 |
| 42342 | 37630410 | 37631242 | 181826 |
| 42343 | 37642934 | 37644138 | 181826 |
| 42344 | 37646292 | 37648169 | 181826 |
| 42345 | 37649294 | 37650798 | 181826 |
| 42346 | 37664174 | 37665541 | 181826 |
| 42347 | 37666213 | 37666844 | 181826 |
| 42348 | 37667698 | 37669262 | 181826 |
| 42349 | 37684070 | 37685328 | 181826 |
| 42350 | 37686693 | 37688846 | 181826 |
| 42351 | 37734937 | 37735598 | |
| 42352 | 37759738 | 37760284 | |
| 42353 | 37814018 | 37814786 | |
| 42354 | 37823672 | 37824349 | |
| 42355 | 37827711 | 37829340 | 169299 |
| 42356 | 37874901 | 37875508 | |
| 42357 | 37891843 | 37893578 | 065882 |
| 42358 | 37972071 | 37972773 | 065882 |
| 42359 | 37977891 | 37980480 | 065882 |
| 42360 | 38076862 | 38077537 | 065882 |
| 42361 | 38089338 | 38090687 | 065882 |
| 42362 | 38129950 | 38131601 | 065882 |
| 42363 | 38132793 | 38136257 | 065882 |
| 42364 | 38142326 | 38143055 | |
| 42365 | 38146490 | 38148713 | |
| 42366 | 38161525 | 38165031 | |
| 42367 | 38171108 | 38172109 | |
| 42368 | 38189669 | 38191673 | |
| 42369 | 38207811 | 38208538 | |
| 42370 | 38224730 | 38227292 | |
| 42371 | 38238594 | 38239247 | |
| 42372 | 38280844 | 38281876 | |
| 42373 | 38309858 | 38311582 | |
| 42374 | 38315155 | 38316851 | |
| 42375 | 38319703 | 38322525 | |
| 42376 | 38385262 | 38385822 | 248227 |
| 42377 | 38386643 | 38389379 | 248227 |
| 42378 | 38492456 | 38493155 | 249534 |
| 42379 | 38576281 | 38577232 | |
| 42380 | 38602902 | 38603581 | |
| 42381 | 38607329 | 38608181 | |
| 42382 | 38664218 | 38667834 | 109787, 196355, 231160 |
| 42383 | 38668011 | 38669989 | 109787 |
| 42384 | 38674594 | 38677668 | 109787 |
| 42385 | 38789114 | 38791250 | |
| 42386 | 38866963 | 38867666 | |
| 42387 | 38868506 | 38870633 | 207944, 197712 |
| 42388 | 38873918 | 38875333 | 197712 |
| 42389 | 38890711 | 38893548 | 197712 |
| 42390 | 38898641 | 38900005 | 197712 |
| 42391 | 38905455 | 38906532 | 197712 |
| 42392 | 38931843 | 38932580 | 197712 |
| 42393 | 38949900 | 38953441 | |
| 42394 | 38966823 | 38970237 | 121895 |
| 42395 | 38991671 | 38992418 | 121895 |
| 42396 | 39033874 | 39034718 | 121895 |
| 42397 | 39044066 | 39048073 | 109790 |
| 42398 | 39183372 | 39184714 | 157796 |
| 42399 | 39233248 | 39234229 | 157796 |
| 42400 | 39326221 | 39327389 | 035928 |
| 42401 | 39339015 | 39340310 | 035928 |
| 42402 | 39342350 | 39343388 | 035928 |
| 42403 | 39350009 | 39351407 | 035928 |
| 42404 | 39367097 | 39369476 | 035928 |
| 42405 | 39408189 | 39409159 | 134962 |
| 42406 | 39411190 | 39412984 | 134962 |
| 42407 | 39413802 | 39416021 | 134962 |
| 42408 | 39417254 | 39417936 | 134962 |
| 42409 | 39435527 | 39436210 | 134962 |
| 42410 | 39451969 | 39452723 | 134962 |
| 42411 | 39459340 | 39461881 | 121897, 163682 |
| 42412 | 39470839 | 39471881 | 121897 |
| 42413 | 39497088 | 39498001 | |
| 42414 | 39515664 | 39518254 | 109814 |
| 42415 | 39527170 | 39531706 | 249348, 109814 |
| 42416 | 39543732 | 39544676 | 249348 |
| 42417 | 39566593 | 39568632 | 163683, 249348 |
| 42418 | 39569925 | 39571536 | 163683, 249348 |
| 42419 | 39610662 | 39611881 | 163683 |
| 42420 | 39622098 | 39624778 | 252796, 163683 |
| 42421 | 39638059 | 39642186 | 255458, 163683 |
| 42422 | 39649871 | 39650206 | 255458 |
| 42423 | 39651024 | 39652405 | 255458 |
| 42424 | 39666028 | 39667267 | 255458 |
| 42425 | 39698528 | 39700962 | 078140 |
| 42426 | 39935194 | 39935724 | 121892 |
| 42427 | 39972046 | 39972740 | 121892 |
| 42428 | 39977901 | 39980104 | 121892 |
| 42429 | 40047558 | 40048528 | 205794 |
| 42430 | 40056954 | 40060333 | 078177, 205794 |
| 42431 | 40256855 | 40258024 | |
| 42432 | 40318931 | 40319631 | 249241 |
| 42433 | 40369336 | 40370452 | |
| 42434 | 40370919 | 40372524 | |
| 42435 | 40377005 | 40377571 | |
| 42436 | 40397584 | 40398452 | |
| 42437 | 40419845 | 40422256 | |
| 42438 | 40448758 | 40449816 | 163694 |
| 42439 | 40456774 | 40458319 | 163694 |
| 42440 | 40459474 | 40461623 | 163694 |
| 42441 | 40475154 | 40476922 | 163694 |
| 42442 | 40488483 | 40489116 | 163694 |
| 42443 | 40493660 | 40494705 | 242197, 163694 |
| 42444 | 40506734 | 40507458 | 163694 |
| 42445 | 40515785 | 40518289 | 163694 |
| 42446 | 40527386 | 40528998 | 163694 |
| 42447 | 40548317 | 40549747 | 163694 |
| 42448 | 40552780 | 40554135 | 163694 |
| 42449 | 40560761 | 40562858 | 163694 |
| 42450 | 40578335 | 40580998 | 163694 |
| 42451 | 40593968 | 40594420 | 163694 |
| 42452 | 40596746 | 40597216 | 163694 |
| 42453 | 40622846 | 40624701 | 163694 |
| 42454 | 40629890 | 40633589 | 163694 |
| 42455 | 40664772 | 40665687 | |
| 42456 | 40689639 | 40691036 | |
| 42457 | 40985978 | 40986954 | 163697 |
| 42458 | 40991769 | 40994357 | 201736, 163697 |
| 42459 | 41079611 | 41080129 | 163697 |
| 42460 | 41112079 | 41112568 | 163697 |
| 42461 | 41122049 | 41123174 | 163697 |
| 42462 | 41134146 | 41134838 | 163697 |
| 42463 | 41144188 | 41148026 | 163697 |
| 42464 | 41148169 | 41150719 | 163697 |
| 42465 | 41177760 | 41179882 | 163697 |
| 42466 | 41206526 | 41207366 | 163697 |
| 42467 | 41214266 | 41217291 | 163697 |
| 42468 | 41217706 | 41219727 | 163697 |
| 42469 | 41320061 | 41323423 | |
| 42470 | 41360383 | 41364178 | 064042 |
| 42471 | 41428542 | 41429006 | 064042 |
| 42472 | 41446251 | 41447068 | 064042 |
| 42473 | 41477324 | 41478167 | 064042 |
| 42474 | 41489903 | 41490308 | 064042 |
| 42475 | 41650326 | 41651000 | 064042 |
| 42476 | 41867260 | 41868182 | |
| 42477 | 41885827 | 41886524 | 249771 |
| 42478 | 41911237 | 41911571 | |
| 42479 | 41935503 | 41938423 | 109133 |
| 42480 | 41940953 | 41941505 | 109133 |
| 42481 | 41992291 | 41993655 | 014824 |
| 42482 | 42658250 | 42660002 | 260519, 124406 |
| 42483 | 44679938 | 44681468 | 151806, 177752 |
| 42484 | 44727840 | 44729062 | 163281 |
| 42485 | 45878217 | 45878863 | |
| 42486 | 46427539 | 46428431 | 151834, 249330 |
| 42487 | 46447742 | 46448437 | 151834 |
| 42488 | 47465084 | 47465982 | 169019 |
| 42489 | 47486836 | 47488622 | 145246 |
| 42490 | 47526936 | 47528082 | 145246 |
| 42491 | 47533249 | 47533975 | 145246 |
| 42492 | 47705600 | 47707390 | 145244 |
| 42493 | 47717803 | 47718767 | 145244 |
| 42494 | 47838338 | 47840117 | 145244 |
| 42495 | 47915143 | 47917191 | 163293, 170448 |
| 42496 | 48017904 | 48019927 | 198515, 163293 |
| 42497 | 48033853 | 48035705 | 163293 |
| 42498 | 48049440 | 48050351 | |
| 42499 | 48095572 | 48096584 | 074966 |
| 42500 | 48110096 | 48112100 | 253050, 074966 |
| 42501 | 48129188 | 48130820 | 074966 |
| 42502 | 48175110 | 48176275 | 135605 |
| 42503 | 48212582 | 48213392 | 135605 |
| 42504 | 48256263 | 48257009 | 135605 |
| 42505 | 48270950 | 48272974 | 135605 |
| 42506 | 48333097 | 48334625 | |
| 42507 | 48342044 | 48344873 | 109171 |
| 42508 | 48377597 | 48378310 | 109171 |
| 42509 | 48636433 | 48637824 | 075539 |
| 42510 | 48780887 | 48783238 | 075539 |
| 42511 | 48832701 | 48834375 | 109180 |
| 42512 | 48906592 | 48909668 | 145247 |
| 42513 | 48911649 | 48912151 | |
| 42514 | 48941706 | 48942460 | |
| 42515 | 52708693 | 52711415 | 109184 |
| 42516 | 52718643 | 52720360 | 109184 |
| 42517 | 52745920 | 52747372 | 109184 |
| 42518 | 52761831 | 52762464 | 109184 |
| 42519 | 52774953 | 52776272 | 109184 |
| 42520 | 52903117 | 52905213 | 163069 |
| 42521 | 52917320 | 52919005 | 163071 |
| 42522 | 53524502 | 53526662 | 248866, 109189 |
| 42523 | 53578148 | 53579987 | 212588, 226950, 264585 |
| 42524 | 53588038 | 53589104 | 226887 |
| 42525 | 53779989 | 53780911 | 184178 |
| 42526 | 53801336 | 53801869 | 184178 |
| 42527 | 53884554 | 53887446 | 184178 |
| 42528 | 53988367 | 53989020 | 184178 |
| 42529 | 54054092 | 54055008 | 184178 |
| 42530 | 54188675 | 54189313 | 184178 |
| 42531 | 54231104 | 54232943 | 184178 |
| 42532 | 54243370 | 54244777 | 145216 |
| 42533 | 54321847 | 54322703 | 145216 |
| 42534 | 54325516 | 54326459 | 145216, 072201 |
| 42535 | 54345127 | 54346171 | 145216, 072201 |
| 42536 | 54372911 | 54375079 | 145216, 072201, 250930 |
| 42537 | 54510682 | 54511846 | 145216, 072201 |
| 42538 | 54519487 | 54520995 | 145216, 072201 |
| 42539 | 54568367 | 54569846 | 145216, 249341 |
| 42540 | 54647114 | 54648880 | 145216 |
| 42541 | 54699165 | 54700108 | 145216 |
| 42542 | 54706839 | 54707318 | 145216 |
| 42543 | 54774519 | 54775583 | 145216, 249706 |
| 42544 | 54794953 | 54795418 | 145216 |
| 42545 | 54802007 | 54802329 | 145216 |
| 42546 | 54928551 | 54932140 | 109220, 145216 |
| 42547 | 54947591 | 54949546 | 145216 |
| 42548 | 54950570 | 54952727 | 145216 |
| 42549 | 54956706 | 54958891 | 145216 |
| 42550 | 55031245 | 55032447 | 145216 |
| 42551 | 55083472 | 55084450 | 145216 |
| 42552 | 55093613 | 55094724 | 145216 |
| 42553 | 55099020 | 55101276 | 145216, 134853 |
| 42554 | 55162639 | 55163389 | 134853 |
| 42555 | 56211939 | 56213783 | 128039 |
| 42556 | 56261356 | 56263702 | 134851, 249700 |
| 42557 | 56334878 | 56335409 | 134852 |
| 42558 | 56411349 | 56414170 | 272969, 134852 |
| 42559 | 56719715 | 56720704 | 090989 |
| 42560 | 56731688 | 56733611 | 090989 |
| 42561 | 56814637 | 56815845 | 174799 |
| 42562 | 57252755 | 57254340 | 269921, 157426 |
| 42563 | 57300780 | 57301704 | 128059 |
| 42564 | 57302004 | 57303708 | 128050 |
| 42565 | 57332250 | 57334911 | 174780 |
| 42566 | 57666090 | 57666734 | |
| 42567 | 57773880 | 57776182 | 084093 |
| 42568 | 57842570 | 57844206 | 047315, 084092 |
| 42569 | 57844459 | 57845801 | 084092, 047315 |
| 42570 | 57848061 | 57848360 | 047315 |
| 42571 | 60418690 | 60419691 | 249392 |
| 42572 | 65879788 | 65880537 | |
| 42573 | 66282430 | 66283019 | 145242 |
| 42574 | 68410292 | 68411858 | 145241 |
| 42575 | 68566046 | 68568535 | 248049, 033178 |
| 42576 | 68579723 | 68580405 | 248049 |
| 42577 | 68590821 | 68591321 | 270292, 248049 |
| 42578 | 69214300 | 69216040 | 083896 |
| 42579 | 69447087 | 69448644 | |
| 42580 | 69463038 | 69463592 | |
| 42581 | 69583173 | 69585589 | |
| 42582 | 69598351 | 69600133 | |
| 42583 | 69616350 | 69617368 | |
| 42584 | 69662506 | 69663773 | |
| 42585 | 69729735 | 69731827 | |
| 42586 | 69743819 | 69744272 | 251529 |
| 42587 | 69769763 | 69775087 | 249985, 272626 |
| 42588 | 69813689 | 69818318 | 135220 |
| 42589 | 69828636 | 69829795 | |
| 42590 | 69898463 | 69899905 | 250919 |
| 42591 | 69922618 | 69925076 | 171234 |
| 42592 | 69932302 | 69933700 | 248763, 171234 |
| 42593 | 69934309 | 69935826 | 248763, 171234 |
| 42594 | 69938373 | 69941701 | 171234 |
| 42595 | 69960454 | 69969050 | 171234 |
| 42596 | 70018813 | 70019459 | |
| 42597 | 70032145 | 70036193 | |
| 42598 | 70324079 | 70324723 | |
| 42599 | 70359014 | 70363062 | 156096 |
| 42600 | 70383322 | 70385062 | 248886, 156096 |
| 42601 | 70410376 | 70412907 | |
| 42602 | 70506041 | 70506455 | 173610, 270386 |
| 42603 | 70579162 | 70580214 | |
| 42604 | 70591733 | 70593588 | 173597 |
| 42605 | 70620314 | 70622220 | 173597 |
| 42606 | 70624676 | 70625563 | 173597 |
| 42607 | 70698042 | 70698803 | 109193 |
| 42608 | 70726241 | 70727019 | |
| 42609 | 71048304 | 71049001 | |
| 42610 | 71450012 | 71450686 | |
| 42611 | 71497012 | 71499071 | 132464 |
| 42612 | 71553100 | 71555343 | 132467 |
| 42613 | 71569341 | 71572666 | 272986, 018189 |
| 42614 | 71578788 | 71581182 | 018189 |
| 42615 | 71704030 | 71705857 | 132463 |
| 42616 | 71767327 | 71769050 | 173542 |
| 42617 | 71858610 | 71860077 | 173542, 156136 |
| 42618 | 72051858 | 72054231 | 080493 |
| 42619 | 72074519 | 72075940 | 080493 |
| 42620 | 72076644 | 72078461 | 080493 |
| 42621 | 72081806 | 72082994 | 080493 |
| 42622 | 72094869 | 72098156 | 080493 |
| 42623 | 72105024 | 72106346 | 080493 |
| 42624 | 72169545 | 72171243 | 080493 |
| 42625 | 72282792 | 72283323 | 080493 |
| 42626 | 72296014 | 72296843 | 080493 |
| 42627 | 72312277 | 72312874 | 080493 |
| 42628 | 72321992 | 72323045 | 080493 |
| 42629 | 72352285 | 72353593 | 080493 |
| 42630 | 72415386 | 72417005 | 080493 |
| 42631 | 72425233 | 72426998 | 080493 |
| 42632 | 72464784 | 72466133 | |
| 42633 | 72473279 | 72475098 | |
| 42634 | 72635043 | 72636831 | 145321 |
| 42635 | 72636981 | 72640276 | 145321 |
| 42636 | 72647343 | 72649129 | 145321 |
| 42637 | 72650516 | 72651184 | 145321 |
| 42638 | 72651410 | 72652402 | 145321 |
| 42639 | 72773306 | 72773944 | |
| 42640 | 72950290 | 72951360 | 056291 |
| 42641 | 72978128 | 72978739 | 056291 |
| 42642 | 73177443 | 73178531 | 156140 |
| 42643 | 73185473 | 73186627 | 156140 |
| 42644 | 73724123 | 73724630 | |
| 42645 | 73761700 | 73762825 | |
| 42646 | 73785474 | 73786221 | |
| 42647 | 73790242 | 73791002 | |
| 42648 | 73933621 | 73936586 | 163626 |
| 42649 | 73969239 | 73971080 | 132466 |
| 42650 | 74122826 | 74126029 | 250220, 132466 |
| 42651 | 74140361 | 74141571 | 250220 |
| 42652 | 74208238 | 74212494 | |
| 42653 | 74240435 | 74242151 | |
| 42654 | 74244526 | 74246312 | |
| 42655 | 74254010 | 74255128 | |
| 42656 | 74262030 | 74267311 | 163631 |
| 42657 | 74269172 | 74276140 | 163631 |
| 42658 | 74281291 | 74282232 | 163631 |
| 42659 | 74287982 | 74292165 | |
| 42660 | 74315704 | 74316928 | 081051 |
| 42661 | 74318653 | 74320190 | 081051 |
| 42662 | 74346543 | 74349313 | 079557 |
| 42663 | 74365354 | 74366865 | 079557 |
| 42664 | 74373188 | 74373742 | |
| 42665 | 74399313 | 74399991 | 250436 |
| 42666 | 74454641 | 74456938 | 169435 |
| 42667 | 74464431 | 74465531 | 169435 |
| 42668 | 74468672 | 74470357 | 169435 |
| 42669 | 74474163 | 74475840 | 169435 |
| 42670 | 74542583 | 74543106 | |
| 42671 | 74569377 | 74571328 | |
| 42672 | 74599859 | 74601025 | |
| 42673 | 74605647 | 74608263 | 169429 |
| 42674 | 74609260 | 74610377 | 169429 |
| 42675 | 74641289 | 74642651 | |
| 42676 | 74698445 | 74699126 | |
| 42677 | 74702130 | 74703558 | 124875 |
| 42678 | 74713798 | 74714772 | 250550, 124875 |
| 42679 | 74721112 | 74723024 | |
| 42680 | 74904133 | 74905196 | 163734 |
| 42681 | 74961041 | 74966651 | 081041 |
| 42682 | 74969674 | 74970882 | |
| 42683 | 74973900 | 74976815 | |
| 42684 | 74982415 | 74983766 | 163738 |
| 42685 | 74988148 | 74989650 | 163738 |
| 42686 | 75022771 | 75025124 | 269559, 163738 |
| 42687 | 75404394 | 75405613 | |
| 42688 | 75410467 | 75411622 | |
| 42689 | 75533324 | 75534301 | |
| 42690 | 75549067 | 75551940 | |
| 42691 | 75552636 | 75553209 | |
| 42692 | 75557115 | 75558391 | |
| 42693 | 75577998 | 75581343 | |
| 42694 | 75584743 | 75586607 | |
| 42695 | 75718914 | 75720219 | 174808 |
| 42696 | 76438575 | 76440668 | 174796, 163743 |
| 42697 | 76467969 | 76468472 | 174796 |
| 42698 | 76596874 | 76599634 | 138757 |
| 42699 | 76627984 | 76628747 | 138757 |
| 42700 | 76648266 | 76651402 | 138768, 138757 |
| 42701 | 76764989 | 76766003 | |
| 42702 | 76816880 | 76817487 | 156194 |
| 42703 | 76840892 | 76841563 | 138744 |
| 42704 | 76843709 | 76844238 | 138744 |
| 42705 | 76860967 | 76862468 | 198301, 138744 |
| 42706 | 76910771 | 76912478 | 198301, 245928 |
| 42707 | 76927460 | 76929083 | 138755 |
| 42708 | 76932713 | 76933720 | 156219 |
| 42709 | 76934404 | 76935210 | 156219 |
| 42710 | 76944490 | 76945291 | 169245, 156219 |
| 42711 | 76949166 | 76950173 | 156219 |
| 42712 | 76956920 | 76957650 | 169248, 156219 |
| 42713 | 77004444 | 77004961 | 156219 |
| 42714 | 77068382 | 77070463 | 138750 |
| 42715 | 77114396 | 77118083 | 138760 |
| 42716 | 77121391 | 77122784 | 138760 |
| 42717 | 77128930 | 77130496 | 138760 |
| 42718 | 77132705 | 77136020 | 189157, 138760 |
| 42719 | 77139809 | 77141416 | 189157 |
| 42720 | 77156756 | 77157641 | 189157 |
| 42721 | 77171983 | 77173476 | 272414, 118804, 189157 |
| 42722 | 77193195 | 77203111 | 272414, 118804, 189157 |
| 42723 | 77213778 | 77214287 | 272414, 118804 |
| 42724 | 77221669 | 77224629 | 272414, 118804 |
| 42725 | 77224695 | 77230350 | 272414, 118804 |
| 42726 | 77232635 | 77234628 | 163749, 118804 |
| 42727 | 77295007 | 77296146 | 163749 |
| 42728 | 77297651 | 77299656 | 163749 |
| 42729 | 77307929 | 77308553 | 163749 |
| 42730 | 77308982 | 77310215 | 163749 |
| 42731 | 77341593 | 77343334 | 163749 |
| 42732 | 77370620 | 77372613 | 138771 |
| 42733 | 77406393 | 77409266 | 138771 |
| 42734 | 77415005 | 77417856 | 138771 |
| 42735 | 77418924 | 77421862 | 138771 |
| 42736 | 77432358 | 77434395 | 138771 |
| 42737 | 77446735 | 77448458 | 138771 |
| 42738 | 77456842 | 77457824 | 138771 |
| 42739 | 77467336 | 77468732 | 138771 |
| 42740 | 77469693 | 77470417 | 138771 |
| 42741 | 77483077 | 77485503 | 138771 |
| 42742 | 77489885 | 77493577 | 138771 |
| 42743 | 77506305 | 77508160 | 138771 |
| 42744 | 77510679 | 77511938 | 138771 |
| 42745 | 77521522 | 77522169 | 138771 |
| 42746 | 77558059 | 77560355 | 231335, 138771 |
| 42747 | 77585757 | 77587376 | 138771 |
| 42748 | 77609790 | 77612364 | 138771 |
| 42749 | 77612774 | 77614289 | 138771 |
| 42750 | 77617328 | 77618028 | 138771 |
| 42751 | 77623267 | 77629612 | 138771 |
| 42752 | 77646124 | 77646647 | 138771 |
| 42753 | 77702576 | 77703906 | 138771, 224218 |
| 42754 | 77730847 | 77731536 | |
| 42755 | 77751458 | 77753882 | |
| 42756 | 77771004 | 77771724 | |
| 42757 | 77777055 | 77778828 | |
| 42758 | 77818036 | 77820724 | 186212 |
| 42759 | 77823467 | 77824513 | |
| 42760 | 77852449 | 77853378 | |
| 42761 | 77864275 | 77864751 | |
| 42762 | 77869374 | 77872532 | 138758 |
| 42763 | 77883709 | 77884237 | 138758 |
| 42764 | 77905726 | 77906734 | 138758 |
| 42765 | 77944659 | 77945454 | 138758 |
| 42766 | 77964893 | 77965281 | |
| 42767 | 77995706 | 77998233 | 118816 |
| 42768 | 78077854 | 78079790 | 138764 |
| 42769 | 78459305 | 78460715 | 156234 |
| 42770 | 78485201 | 78486464 | 156234 |
| 42771 | 78500617 | 78501193 | 156234 |
| 42772 | 78552315 | 78553002 | |
| 42773 | 78571344 | 78572600 | |
| 42774 | 78619008 | 78619944 | |
| 42775 | 78737610 | 78738245 | 138767 |
| 42776 | 78738450 | 78743091 | 138767 |
| 42777 | 78782852 | 78784855 | 169288 |
| 42778 | 78842678 | 78843269 | 169288 |
| 42779 | 79490325 | 79490941 | 138772 |
| 42780 | 79566292 | 79568020 | 251442 |
| 42781 | 79570651 | 79572531 | 251442 |
| 42782 | 79582408 | 79583625 | 251442 |
| 42783 | 79695855 | 79698590 | 138756, 260278 |
| 42784 | 79783025 | 79784148 | 138756 |
| 42785 | 79859719 | 79861080 | 163291 |
| 42786 | 80214988 | 80215824 | 249307, 156269 |
| 42787 | 80926964 | 80927657 | 163297 |
| 42788 | 80992923 | 80994605 | 163297 |
| 42789 | 81973125 | 81974075 | 152785 |
| 42790 | 82085061 | 82086416 | 251059, 138669 |
| 42791 | 82388123 | 82394235 | 138670 |
| 42792 | 82414762 | 82415934 | 138670 |
| 42793 | 82484815 | 82488609 | 138670 |
| 42794 | 82497794 | 82500959 | 138670 |
| 42795 | 82517272 | 82517774 | 138670 |
| 42796 | 82540735 | 82545128 | 138670, 251331 |
| 42797 | 82545337 | 82547855 | 138670, 251331 |
| 42798 | 82548165 | 82550795 | 138670, 251331 |
| 42799 | 82552014 | 82554621 | 138670, 251331 |
| 42800 | 82555428 | 82557710 | 138670, 251331 |
| 42801 | 82571461 | 82573691 | 138670, 251331 |
| 42802 | 82644344 | 82646911 | 138670, 251331 |
| 42803 | 82659079 | 82660459 | 138670, 251331 |
| 42804 | 82760735 | 82761272 | 138670, 251331 |
| 42805 | 83293818 | 83296704 | 272677, 138668 |
| 42806 | 83349855 | 83352665 | 145293, 152795 |
| 42807 | 83440087 | 83440597 | 249242 |
| 42808 | 83452728 | 83453564 | 249242 |
| 42809 | 83482271 | 83483858 | 249242 |
| 42810 | 83755071 | 83755467 | 138674 |
| 42811 | 83810744 | 83812792 | 138674 |
| 42812 | 83820599 | 83823132 | 168152, 251022, 138674 |
| 42813 | 83930948 | 83932629 | 189308 |
| 42814 | 83933272 | 83935380 | 189308 |
| 42815 | 83955351 | 83957191 | 138663 |
| 42816 | 84030658 | 84032061 | 145287 |
| 42817 | 84176639 | 84178643 | |
| 42818 | 84205281 | 84206659 | 173085 |
| 42819 | 84278347 | 84278758 | |
| 42820 | 84375971 | 84378193 | 163319, 163312 |
| 42821 | 84405620 | 84406974 | 163322 |
| 42822 | 84456307 | 84458479 | 138678 |
| 42823 | 84464700 | 84465249 | 138678 |
| 42824 | 84468641 | 84470733 | 138678 |
| 42825 | 84482471 | 84483101 | 138678 |
| 42826 | 84495372 | 84495920 | 138678 |
| 42827 | 84503387 | 84504095 | 138678 |
| 42828 | 84539375 | 84540633 | |
| 42829 | 85734913 | 85736717 | 163625 |
| 42830 | 85872871 | 85873665 | 163625 |
| 42831 | 85886678 | 85888910 | 180769, 163625 |
| 42832 | 86396051 | 86396605 | 138639 |
| 42833 | 86430999 | 86431538 | 138639 |
| 42834 | 86699318 | 86700311 | 138639 |
| 42835 | 86707756 | 86708775 | 138639 |
| 42836 | 86747205 | 86747897 | 138639 |
| 42837 | 86748356 | 86750049 | 138639 |
| 42838 | 86849891 | 86851783 | 138639 |
| 42839 | 87812479 | 87814160 | 163633 |
| 42840 | 87839652 | 87840450 | 163633 |
| 42841 | 87845424 | 87846400 | 163633 |
| 42842 | 87847592 | 87850700 | 163633 |
| 42843 | 87855067 | 87858978 | 172493, 163633 |
| 42844 | 87862764 | 87863947 | 172493 |
| 42845 | 87884720 | 87885214 | 172493 |
| 42846 | 87894883 | 87895327 | 172493 |
| 42847 | 87898624 | 87899368 | 172493 |
| 42848 | 87915021 | 87916000 | 172493 |
| 42849 | 87927255 | 87929483 | 172493 |
| 42850 | 87933651 | 87934239 | 172493 |
| 42851 | 87954351 | 87955420 | 172493 |
| 42852 | 87962240 | 87962682 | 172493 |
| 42853 | 88013635 | 88016271 | 172493 |
| 42854 | 88029103 | 88029902 | 172493 |
| 42855 | 88038208 | 88038972 | 172493 |
| 42856 | 88041252 | 88043833 | 172493 |
| 42857 | 88086874 | 88087702 | 145332 |
| 42858 | 88109289 | 88110729 | 145332 |
| 42859 | 88140608 | 88142294 | 145332 |
| 42860 | 88156346 | 88157795 | 145332 |
| 42861 | 88199277 | 88200099 | |
| 42862 | 88238275 | 88239191 | 255723, 170509 |
| 42863 | 88277791 | 88279087 | 198189 |
| 42864 | 88310543 | 88313366 | 198189 |
| 42865 | 88329713 | 88331200 | |
| 42866 | 88337749 | 88338794 | |
| 42867 | 88340048 | 88345207 | 170502 |
| 42868 | 88411947 | 88412699 | 152583 |
| 42869 | 88413596 | 88414626 | 152583 |
| 42870 | 88510443 | 88511226 | 249001 |
| 42871 | 88786044 | 88788230 | |
| 42872 | 88831602 | 88833104 | |
| 42873 | 88833192 | 88834477 | |
| 42874 | 88883060 | 88883962 | |
| 42875 | 88895924 | 88896573 | |
| 42876 | 88928532 | 88929832 | 118762 |
| 42877 | 88938947 | 88939456 | 118762 |
| 42878 | 88983723 | 88984464 | 118762 |
| 42879 | 89027491 | 89030082 | 118777 |
| 42880 | 89031904 | 89032548 | 118777 |
| 42881 | 89079467 | 89080759 | 118777 |
| 42882 | 89083619 | 89084270 | 118777 |
| 42883 | 89204401 | 89206440 | 246375, 163644 |
| 42884 | 89299764 | 89300821 | 138642 |
| 42885 | 89377725 | 89378770 | 138646 |
| 42886 | 89387734 | 89388328 | 138646 |
| 42887 | 89443704 | 89445720 | 145337, 138641 |
| 42888 | 89512483 | 89514727 | 138641 |
| 42889 | 89532264 | 89535269 | 138641 |
| 42890 | 89543452 | 89545162 | 138641 |
| 42891 | 89626912 | 89628252 | 138641 |
| 42892 | 89654150 | 89657747 | 138640 |
| 42893 | 89657768 | 89659036 | 138640 |
| 42894 | 89673718 | 89676421 | 138640 |
| 42895 | 89686892 | 89687495 | 138640 |
| 42896 | 89713025 | 89714423 | 138640 |
| 42897 | 89730501 | 89731477 | 138640 |
| 42898 | 89744112 | 89745225 | 138640 |
| 42899 | 89930235 | 89930686 | 138640 |
| 42900 | 90031652 | 90033893 | 271359, 138640 |
| 42901 | 90034444 | 90035669 | 180346 |
| 42902 | 90043564 | 90045260 | |
| 42903 | 90117723 | 90118455 | |
| 42904 | 90194751 | 90196239 | 185477 |
| 42905 | 90196514 | 90197282 | 185477 |
| 42906 | 90204259 | 90204897 | 185477 |
| 42907 | 90214240 | 90215044 | 185477 |
| 42908 | 90217262 | 90218351 | 185477 |
| 42909 | 90219842 | 90220813 | 185477 |
| 42910 | 92835235 | 92835891 | |
| 42911 | 93889790 | 93892088 | 152208 |
| 42912 | 93909462 | 93910009 | 152208 |
| 42913 | 93913051 | 93913598 | 152208 |
| 42914 | 95128319 | 95129827 | 163104, 246541 |
| 42915 | 95219932 | 95220538 | 163106 |
| 42916 | 95330918 | 95332903 | |
| 42917 | 95333082 | 95334693 | |
| 42918 | 95340944 | 95341546 | |
| 42919 | 95372220 | 95377292 | 163110 |
| 42920 | 95393267 | 95395266 | 163110 |
| 42921 | 95427823 | 95430028 | 163110 |
| 42922 | 95436097 | 95439127 | 163110 |
| 42923 | 95468795 | 95470721 | 163110 |
| 42924 | 95485347 | 95485889 | 163110 |
| 42925 | 95562245 | 95564476 | 163110 |
| 42926 | 98396516 | 98397112 | 251620, 163116, 254044 |
| 42927 | 98414829 | 98415814 | 163116, 254044 |
| 42928 | 98417111 | 98418712 | 163116, 254044 |
| 42929 | 98419967 | 98420642 | 163116, 254044 |
| 42930 | 98787953 | 98788612 | 163116 |
| 42931 | 99063824 | 99065356 | 163116 |
| 42932 | 99087283 | 99088391 | |
| 42933 | 99174316 | 99175228 | 214559 |
| 42934 | 99180712 | 99184180 | 138698, 214559 |
| 42935 | 99547370 | 99547764 | 168785 |
| 42936 | 99564181 | 99564805 | 168785 |
| 42937 | 99699241 | 99699868 | |
| 42938 | 99849024 | 99852122 | 238449, 263923, 151247 |
| 42939 | 99858963 | 99859932 | 263923 |
| 42940 | 99915781 | 99918051 | 164024, 263923 |
| 42941 | 100007986 | 100010985 | 246090, 197894 |
| 42942 | 100064823 | 100066014 | 198099, 246090 |
| 42943 | 100077417 | 100079157 | 198099, 246090 |
| 42944 | 100125952 | 100127162 | 172955, 246090 |
| 42945 | 100139766 | 100141323 | 172955, 246090 |
| 42946 | 100212018 | 100212833 | 187758, 246090 |
| 42947 | 100244644 | 100245568 | |
| 42948 | 100273702 | 100274573 | 248144 |
| 42949 | 100301698 | 100306467 | |
| 42950 | 100323726 | 100327122 | |
| 42951 | 100334153 | 100334828 | 196344 |
| 42952 | 100335354 | 100336088 | 196344 |
| 42953 | 100380476 | 100381376 | |
| 42954 | 100382652 | 100383486 | |
| 42955 | 100483879 | 100485523 | 138823, 145331 |
| 42956 | 100493044 | 100494519 | 138823 |
| 42957 | 100494832 | 100497831 | 138823 |
| 42958 | 100507337 | 100509828 | 138823 |
| 42959 | 100533334 | 100534353 | 138823, 248676 |
| 42960 | 100563888 | 100565566 | 248713 |
| 42961 | 100600125 | 100601604 | |
| 42962 | 100643312 | 100645655 | |
| 42963 | 100684746 | 100685532 | |
| 42964 | 100814525 | 100815900 | 109270 |
| 42965 | 100866642 | 100868671 | 164031 |
| 42966 | 100869946 | 100871169 | 245322, 164032 |
| 42967 | 100871186 | 100872492 | 164032, 245322 |
| 42968 | 101744611 | 101745210 | 164035 |
| 42969 | 101760722 | 101761437 | 164035 |
| 42970 | 101816293 | 101817517 | |
| 42971 | 101850126 | 101850937 | |
| 42972 | 101898624 | 101899344 | |
| 42973 | 101907030 | 101908236 | |
| 42974 | 101957821 | 101959647 | 138814 |
| 42975 | 102090003 | 102090996 | 138814 |
| 42976 | 102104655 | 102105316 | 138814 |
| 42977 | 102112293 | 102113210 | 138814 |
| 42978 | 102120098 | 102120726 | 138814 |
| 42979 | 102132207 | 102132728 | 138814 |
| 42980 | 102206589 | 102207190 | 138814 |
| 42981 | 102266749 | 102270322 | 254531, 138814 |
| 42982 | 102276921 | 102279356 | |
| 42983 | 102314218 | 102314872 | |
| 42984 | 103264758 | 103267242 | 138821 |
| 42985 | 103277662 | 103278512 | 138821 |
| 42986 | 103348451 | 103349785 | 138821, 248161 |
| 42987 | 103350280 | 103351232 | 138821, 248161 |
| 42988 | 103359507 | 103361862 | 248161 |
| 42989 | 103362899 | 103364429 | 248161 |
| 42990 | 103421224 | 103423804 | 260651, 109320 |
| 42991 | 103540487 | 103543948 | |
| 42992 | 103608754 | 103610093 | 109323 |
| 42993 | 103633894 | 103635369 | 109323 |
| 42994 | 103637798 | 103639813 | 109323 |
| 42995 | 103681020 | 103682872 | 109323 |
| 42996 | 103699887 | 103700382 | |
| 42997 | 103701481 | 103702564 | |
| 42998 | 103745420 | 103750715 | 246560, 109332 |
| 42999 | 103789140 | 103791490 | 145354, 109332 |
| 43000 | 103799418 | 103800978 | 145354 |
| 43001 | 103939792 | 103941714 | 164038, 164037 |
| 43002 | 103988489 | 103990291 | 164038 |
| 43003 | 103995886 | 103998727 | 164038 |
| 43004 | 104018293 | 104019419 | 164039 |
| 43005 | 104020005 | 104021443 | 164039 |
| 43006 | 104119284 | 104120138 | 138778 |
| 43007 | 104343470 | 104344905 | 248740 |
| 43008 | 104345882 | 104347098 | 248740 |
| 43009 | 105049059 | 105050891 | |
| 43010 | 105411527 | 105413821 | 245384, 272082, 168772 |
| 43011 | 105414957 | 105416818 | 168772, 245384 |
| 43012 | 105516904 | 105517774 | 245384 |
| 43013 | 105648158 | 105650590 | 248242 |
| 43014 | 105668404 | 105669444 | 248242 |
| 43015 | 105836423 | 105837439 | 248242, 248373 |
| 43016 | 106067152 | 106069428 | 168769 |
| 43017 | 106079953 | 106080451 | 168769 |
| 43018 | 106100439 | 106101797 | 168769 |
| 43019 | 106107818 | 106108474 | 168769 |
| 43020 | 106338539 | 106339314 | 138777 |
| 43021 | 106343376 | 106343837 | 138777 |
| 43022 | 106392662 | 106395598 | 138777 |
| 43023 | 106507014 | 106508345 | 236699 |
| 43024 | 106514434 | 106516789 | 236699 |
| 43025 | 106519211 | 106520311 | 236699 |
| 43026 | 106526456 | 106527209 | 236699 |
| 43027 | 106541805 | 106542579 | 236699 |
| 43028 | 106545079 | 106546396 | 236699 |
| 43029 | 106591735 | 106592354 | 236699 |
| 43030 | 106628790 | 106630422 | 138780, 236699, 138785 |
| 43031 | 107208329 | 107209108 | 145348 |
| 43032 | 107236215 | 107238873 | 164022, 145348 |
| 43033 | 107286703 | 107287411 | 250298 |
| 43034 | 107342755 | 107344145 | |
| 43035 | 108104142 | 108104920 | 155011 |
| 43036 | 108554182 | 108554817 | 138801 |
| 43037 | 108604975 | 108605433 | 138801 |
| 43038 | 108610685 | 108611749 | 138801 |
| 43039 | 108640734 | 108642034 | 138801 |
| 43040 | 108744670 | 108748127 | 164023 |
| 43041 | 108759022 | 108760442 | 164023 |
| 43042 | 108774091 | 108775083 | 164023 |
| 43043 | 108783859 | 108786051 | 245293, 164023 |
| 43044 | 108816617 | 108818549 | 245293, 164023 |
| 43045 | 108851875 | 108853492 | 155016, 245293 |
| 43046 | 108909303 | 108912251 | 138796 |
| 43047 | 108928954 | 108931362 | 138796 |
| 43048 | 109540983 | 109543044 | 109475, 234492 |
| 43049 | 109615100 | 109616036 | |
| 43050 | 109651625 | 109652331 | |
| 43051 | 109682853 | 109686236 | 164089 |
| 43052 | 109686926 | 109687877 | |
| 43053 | 109695813 | 109696563 | |
| 43054 | 109712321 | 109712961 | |
| 43055 | 109715131 | 109715880 | |
| 43056 | 109749812 | 109750400 | 188517 |
| 43057 | 110353965 | 110357429 | 138802, 247950 |
| 43058 | 110481081 | 110482699 | 005059 |
| 43059 | 110623511 | 110625292 | 138794 |
| 43060 | 110649845 | 110652090 | 123739 |
| 43061 | 110722003 | 110724069 | 205403 |
| 43062 | 110736448 | 110737911 | 109534, 272795 |
| 43063 | 110866217 | 110866663 | 138798 |
| 43064 | 110929503 | 110930248 | 138798 |
| 43065 | 110975679 | 110977583 | 170522 |
| 43066 | 111027386 | 111029055 | 170522 |
| 43067 | 111052915 | 111055099 | 170522 |
| 43068 | 111079938 | 111081729 | 170522 |
| 43069 | 111083613 | 111087720 | 170522 |
| 43070 | 111114712 | 111116082 | 170522 |
| 43071 | 111116551 | 111121139 | 170522 |
| 43072 | 111170862 | 111171552 | |
| 43073 | 111268787 | 111269833 | |
| 43074 | 111269952 | 111270694 | |
| 43075 | 111315909 | 111316462 | 138792 |
| 43076 | 111370903 | 111371678 | 138792 |
| 43077 | 111396985 | 111398358 | 138792 |
| 43078 | 111408905 | 111409392 | 138792 |
| 43079 | 111436821 | 111437983 | 250511, 138792 |
| 43080 | 111454599 | 111455649 | 138792 |
| 43081 | 111461613 | 111462947 | 138792 |
| 43082 | 111790976 | 111791513 | |
| 43083 | 113004055 | 113005728 | |
| 43084 | 113033454 | 113035308 | |
| 43085 | 113061593 | 113062110 | |
| 43086 | 113066382 | 113068182 | 174749 |
| 43087 | 113151554 | 113154083 | 138660, 260526 |
| 43088 | 113171473 | 113172561 | 138660 |
| 43089 | 113177963 | 113178732 | 138660 |
| 43090 | 113205929 | 113207797 | 073331, 145365 |
| 43091 | 113218123 | 113219188 | 073331 |
| 43092 | 113557968 | 113559298 | 174720, 138658 |
| 43093 | 113990249 | 113991386 | 145362, 248152 |
| 43094 | 114030173 | 114031023 | 145362 |
| 43095 | 114062915 | 114063657 | 145362 |
| 43096 | 114066784 | 114067622 | 145362 |
| 43097 | 114067829 | 114069415 | 145362 |
| 43098 | 114325044 | 114326741 | |
| 43099 | 114327250 | 114328507 | |
| 43100 | 114331786 | 114333184 | |
| 43101 | 114354340 | 114356390 | |
| 43102 | 114366344 | 114368557 | |
| 43103 | 114388145 | 114391757 | 145349 |
| 43104 | 114420822 | 114421246 | 145349 |
| 43105 | 114468567 | 114469282 | 145349 |
| 43106 | 114495629 | 114496104 | 145349 |
| 43107 | 114561908 | 114562785 | 145349 |
| 43108 | 114566826 | 114567558 | 145349 |
| 43109 | 114580009 | 114580767 | 145349 |
| 43110 | 114587829 | 114588450 | 145349 |
| 43111 | 114680487 | 114684985 | 145349 |
| 43112 | 114705439 | 114706499 | |
| 43113 | 114822709 | 114823598 | 180801 |
| 43114 | 114879483 | 114884241 | 180801 |
| 43115 | 114892492 | 114893311 | 180801 |
| 43116 | 114896626 | 114897477 | 180801 |
| 43117 | 114899619 | 114901774 | 180801 |
| 43118 | 114926838 | 114927804 | |
| 43119 | 114940795 | 114941335 | |
| 43120 | 114978234 | 114979597 | |
| 43121 | 115208294 | 115208867 | |
| 43122 | 116765042 | 116766142 | |
| 43123 | 117783663 | 117784255 | |
| 43124 | 119198047 | 119204554 | 269893, 164099 |
| 43125 | 119506598 | 119508266 | |
| 43126 | 119605892 | 119607356 | 145388 |
| 43127 | 119639729 | 119640402 | |
| 43128 | 119678376 | 119679566 | 150961 |
| 43129 | 119731710 | 119733576 | 150961 |
| 43130 | 119741950 | 119745101 | 150961 |
| 43131 | 119747508 | 119749331 | 150961 |
| 43132 | 119755942 | 119758504 | 150961 |
| 43133 | 119778886 | 119779395 | |
| 43134 | 119805840 | 119806379 | |
| 43135 | 119838636 | 119840709 | 172403 |
| 43136 | 119889594 | 119890903 | 172403 |
| 43137 | 119969512 | 119970210 | 172403 |
| 43138 | 120001198 | 120002894 | |
| 43139 | 120023820 | 120025482 | |
| 43140 | 120132368 | 120134942 | 145390, 178636 |
| 43141 | 120219931 | 120222578 | 164096 |
| 43142 | 120399635 | 120400453 | 245958 |
| 43143 | 120438595 | 120439949 | 245958, 138735 |
| 43144 | 120445604 | 120447746 | 245958, 138735 |
| 43145 | 120548160 | 120549454 | 138735 |
| 43146 | 120549676 | 120550875 | 138735 |
| 43147 | 120569479 | 120570068 | |
| 43148 | 120604640 | 120605212 | |
| 43149 | 120628281 | 120629141 | |
| 43150 | 120650128 | 120652019 | |
| 43151 | 120652201 | 120652722 | |
| 43152 | 120987836 | 120989545 | 250938, 164109 |
| 43153 | 121955196 | 121955646 | |
| 43154 | 122610149 | 122613815 | 164111 |
| 43155 | 122615897 | 122619722 | 164111 |
| 43156 | 122632227 | 122633533 | |
| 43157 | 122721650 | 122722805 | 123737 |
| 43158 | 122744679 | 122745537 | 145386 |
| 43159 | 123072837 | 123074460 | 138688 |
| 43160 | 123653307 | 123654803 | 181004, 224786 |
| 43161 | 123684936 | 123689036 | |
| 43162 | 123702699 | 123709693 | |
| 43163 | 123717511 | 123720436 | |
| 43164 | 123740427 | 123742259 | |
| 43165 | 123747406 | 123750001 | 138685, 253069 |
| 43166 | 123763648 | 123764552 | 138685 |
| 43167 | 123794943 | 123796418 | 138685 |
| 43168 | 123819402 | 123820007 | 170917 |
| 43169 | 123830683 | 123831254 | 170917 |
| 43170 | 123841120 | 123844852 | 145375, 170917 |
| 43171 | 123985532 | 123986074 | 145375 |
| 43172 | 124272659 | 124274084 | |
| 43173 | 124317543 | 124321221 | 164056 |
| 43174 | 124333999 | 124335079 | |
| 43175 | 124399881 | 124401391 | |
| 43176 | 124466452 | 124467912 | |
| 43177 | 124475674 | 124477805 | |
| 43178 | 124520030 | 124521634 | |
| 43179 | 125451205 | 125452396 | 261083 |
| 43180 | 125632584 | 125634940 | 151458 |
| 43181 | 126235301 | 126237206 | |
| 43182 | 126242671 | 126246030 | 196159 |
| 43183 | 126289627 | 126290146 | 196159 |
| 43184 | 126310618 | 126311832 | 196159 |
| 43185 | 126640791 | 126641678 | 250149 |
| 43186 | 128454400 | 128454910 | |
| 43187 | 128553650 | 128554756 | 164066 |
| 43188 | 128630068 | 128630591 | 164066 |
| 43189 | 128702648 | 128705460 | 164070 |
| 43190 | 128801623 | 128802975 | 142731 |
| 43191 | 128885664 | 128887428 | 164074, 164073 |
| 43192 | 128896199 | 128897047 | 164074 |
| 43193 | 128981123 | 128984208 | 138709 |
| 43194 | 128986696 | 128987305 | 138709 |
| 43195 | 129083157 | 129086044 | 138709 |
| 43196 | 129161746 | 129163760 | |
| 43197 | 129205452 | 129210652 | 164040 |
| 43198 | 129219977 | 129220643 | 251432 |
| 43199 | 129231259 | 129232014 | 251432 |
| 43200 | 129240921 | 129241421 | 251432 |
| 43201 | 129270808 | 129271486 | 251432 |
| 43202 | 129307982 | 129308705 | 251432 |
| 43203 | 129348906 | 129349646 | 251432 |
| 43204 | 129374733 | 129376910 | 251432 |
| 43205 | 129437765 | 129440172 | 251432 |
| 43206 | 129441788 | 129443404 | |
| 43207 | 129448715 | 129451205 | |
| 43208 | 129472301 | 129476746 | 273077 |
| 43209 | 129491013 | 129496530 | 248187 |
| 43210 | 129505053 | 129506109 | 248802 |
| 43211 | 129521813 | 129523865 | 248802 |
| 43212 | 129556273 | 129557273 | |
| 43213 | 129703201 | 129705049 | |
| 43214 | 129714608 | 129715799 | |
| 43215 | 129717829 | 129719209 | |
| 43216 | 129730124 | 129734355 | 077684 |
| 43217 | 129744083 | 129744916 | 077684 |
| 43218 | 130013896 | 130015071 | 151470, 151466 |
| 43219 | 130017002 | 130017878 | 151470 |
| 43220 | 130110573 | 130111783 | |
| 43221 | 130114029 | 130115254 | |
| 43222 | 130159836 | 130160837 | |
| 43223 | 130263323 | 130265812 | |
| 43224 | 130993935 | 130994954 | |
| 43225 | 137846969 | 137849468 | 248869 |
| 43226 | 139120390 | 139121572 | 151012 |
| 43227 | 139818807 | 139819568 | 250195 |
| 43228 | 139860849 | 139861832 | 250195 |
| 43229 | 139874945 | 139875738 | 250195 |
| 43230 | 139888421 | 139889057 | 250195 |
| 43231 | 139902941 | 139903833 | 250195 |
| 43232 | 139934549 | 139938373 | 151014 |
| 43233 | 139938597 | 139939418 | 264953, 151014 |
| 43234 | 139948796 | 139949989 | 109381, 151014 |
| 43235 | 140003261 | 140006500 | 109381 |
| 43236 | 140096805 | 140100030 | 109381 |
| 43237 | 140163838 | 140165588 | |
| 43238 | 140187511 | 140188434 | 109390, 137463 |
| 43239 | 140215897 | 140217369 | 109390 |
| 43240 | 140221294 | 140223659 | 164134, 109390 |
| 43241 | 140373975 | 140376033 | 172007, 273247 |
| 43242 | 140377016 | 140378973 | 172007 |
| 43243 | 140417668 | 140418522 | 145391 |
| 43244 | 140476063 | 140480786 | 272717, 145391 |
| 43245 | 140513743 | 140514910 | 145391 |
| 43246 | 140520791 | 140521509 | 145391 |
| 43247 | 140561920 | 140563040 | |
| 43248 | 140577610 | 140579373 | |
| 43249 | 140586341 | 140588754 | 085871 |
| 43250 | 140597618 | 140598813 | 085871 |
| 43251 | 140609511 | 140611187 | 085871 |
| 43252 | 140613043 | 140614483 | 085871 |
| 43253 | 140655178 | 140657529 | 196782, 085871 |
| 43254 | 140660868 | 140662264 | 085871, 196782 |
| 43255 | 140719489 | 140720206 | 196782 |
| 43256 | 140735450 | 140736303 | 196782 |
| 43257 | 140762520 | 140763666 | 196782 |
| 43258 | 140828129 | 140828952 | 196782 |
| 43259 | 140896798 | 140897602 | 196782 |
| 43260 | 140939871 | 140943305 | 196782 |
| 43261 | 140958469 | 140958790 | 196782 |
| 43262 | 140982126 | 140983193 | 196782 |
| 43263 | 141071085 | 141076168 | 196782 |
| 43264 | 141148360 | 141148883 | |
| 43265 | 141149699 | 141150944 | |
| 43266 | 141159081 | 141160392 | |
| 43267 | 141172089 | 141174805 | |
| 43268 | 141176720 | 141179346 | 153130 |
| 43269 | 141283857 | 141284646 | 196951, 153130 |
| 43270 | 141293837 | 141295722 | 196951, 153130 |
| 43271 | 141347637 | 141349662 | 153132 |
| 43272 | 141444587 | 141446191 | 179387 |
| 43273 | 141556694 | 141557251 | 109436 |
| 43274 | 141562240 | 141563523 | 249661, 109436 |
| 43275 | 141566737 | 141567864 | 109436 |
| 43276 | 141587971 | 141588705 | 109436 |
| 43277 | 141645418 | 141646594 | 109436 |
| 43278 | 141647517 | 141648831 | 109436 |
| 43279 | 141649096 | 141649850 | 109436 |
| 43280 | 141663546 | 141664113 | 109436 |
| 43281 | 141676582 | 141678799 | 273472, 109436 |
| 43282 | 141927551 | 141927959 | 170153 |
| 43283 | 142116238 | 142116863 | 170153 |
| 43284 | 142141702 | 142142985 | 109445 |
| 43285 | 142254852 | 142255478 | |
| 43286 | 142268837 | 142269787 | |
| 43287 | 142271016 | 142272002 | |
| 43288 | 142557180 | 142558842 | 164136 |
| 43289 | 142563173 | 142565728 | 164136 |
| 43290 | 142599626 | 142600910 | 164136 |
| 43291 | 142634979 | 142635685 | 164136 |
| 43292 | 142770504 | 142771374 | |
| 43293 | 142774435 | 142775007 | |
| 43294 | 143186472 | 143187694 | 109452 |
| 43295 | 143516335 | 143517221 | 109452, 249806 |
| 43296 | 144104762 | 144108628 | 170185, 250326 |
| 43297 | 144146134 | 144146867 | |
| 43298 | 144256550 | 144259866 | 109458, 178972 |
| 43299 | 144276687 | 144277340 | 109458 |
| 43300 | 144307914 | 144308437 | 109458 |
| 43301 | 144310607 | 144311646 | 109458 |
| 43302 | 144433822 | 144436306 | 153147, 245112 |
| 43303 | 146018454 | 146020693 | 164163, 164162 |
| 43304 | 146098360 | 146102623 | 164164 |
| 43305 | 146260196 | 146261045 | 272727 |
| 43306 | 146338776 | 146339441 | |
| 43307 | 146341291 | 146342710 | |
| 43308 | 146360407 | 146361144 | |
| 43309 | 146361265 | 146362058 | |
| 43310 | 146402185 | 146404779 | 170365 |
| 43311 | 146418706 | 146419326 | 250582, 170365 |
| 43312 | 146428549 | 146429897 | 170365 |
| 43313 | 146466309 | 146467470 | 170365 |
| 43314 | 146539306 | 146542189 | 151611 |
| 43315 | 146561388 | 146561827 | 151611 |
| 43316 | 146584218 | 146584713 | |
| 43317 | 146653452 | 146658237 | 237136 |
| 43318 | 146706521 | 146707344 | 151612 |
| 43319 | 146724995 | 146727422 | 151612 |
| 43320 | 146728789 | 146730515 | 151612 |
| 43321 | 146857374 | 146858601 | 151612 |
| 43322 | 146858966 | 146860334 | 151612 |
| 43323 | 147096507 | 147097824 | 164167 |
| 43324 | 147300819 | 147301278 | 120519 |
| 43325 | 147416339 | 147417130 | 120519 |
| 43326 | 147441844 | 147444278 | 120519 |
| 43327 | 148604314 | 148606680 | 164169 |
| 43328 | 148652463 | 148654454 | 071205 |
| 43329 | 148657514 | 148658027 | 071205 |
| 43330 | 148752415 | 148754010 | 071205 |
| 43331 | 148785713 | 148786207 | 071205 |
| 43332 | 148850981 | 148851451 | 071205 |
| 43333 | 149124209 | 149125079 | 250354, 151623 |
| 43334 | 149174833 | 149175620 | 151623 |
| 43335 | 149191695 | 149192431 | 151623 |
| 43336 | 149362574 | 149364315 | 151623 |
| 43337 | 149364676 | 149367045 | 151623 |
| 43338 | 149770765 | 149772220 | |
| 43339 | 149775236 | 149775935 | |
| 43340 | 150325170 | 150325836 | 234828 |
| 43341 | 150330082 | 150330975 | 234828 |
| 43342 | 150414241 | 150415510 | 234828 |
| 43343 | 150452814 | 150453437 | 234828 |
| 43344 | 150918985 | 150919773 | |
| 43345 | 151040424 | 151041192 | 170390 |
| 43346 | 151223620 | 151224139 | 198589 |
| 43347 | 151422083 | 151422538 | 198589 |
| 43348 | 151435789 | 151436637 | 198589 |
| 43349 | 151438898 | 151440140 | 198589 |
| 43350 | 151504118 | 151505589 | 181541, 198589 |
| 43351 | 151644040 | 151644659 | 198589 |
| 43352 | 151689168 | 151691864 | 198589 |
| 43353 | 151936108 | 151938221 | 198589 |
| 43354 | 152020043 | 152021961 | 145425 |
| 43355 | 152105668 | 152106697 | 109686 |
| 43356 | 152126214 | 152126716 | 109686 |
| 43357 | 152146784 | 152147758 | 109686 |
| 43358 | 152169411 | 152170148 | 109686 |
| 43359 | 152172975 | 152173575 | 109686 |
| 43360 | 152234249 | 152238431 | 109686 |
| 43361 | 152244732 | 152247615 | 109686 |
| 43362 | 152329662 | 152331233 | 164142, 251611 |
| 43363 | 152338612 | 152340833 | 164142 |
| 43364 | 152355520 | 152356231 | 164142 |
| 43365 | 152405695 | 152407354 | 164142 |
| 43366 | 152665509 | 152666411 | 059691 |
| 43367 | 152681249 | 152682605 | 059691 |
| 43368 | 153277468 | 153278064 | 109670 |
| 43369 | 153402069 | 153402472 | 109670 |
| 43370 | 153406230 | 153407646 | 109670 |
| 43371 | 153407996 | 153408838 | 109670 |
| 43372 | 153455693 | 153458929 | 268471, 109670 |
| 43373 | 153500225 | 153500773 | |
| 43374 | 153600531 | 153601338 | 170006 |
| 43375 | 153700440 | 153701986 | 164144, 169989 |
| 43376 | 153732003 | 153732387 | 164144 |
| 43377 | 153770769 | 153772905 | 164144 |
| 43378 | 153878126 | 153879228 | 137460 |
| 43379 | 153988397 | 153989221 | 249022 |
| 43380 | 154036096 | 154037614 | |
| 43381 | 154064010 | 154065429 | |
| 43382 | 154143344 | 154144969 | 109654 |
| 43383 | 154154784 | 154155456 | 109654 |
| 43384 | 154170129 | 154170815 | 109654 |
| 43385 | 154190694 | 154191166 | 109654 |
| 43386 | 154195709 | 154196681 | 109654 |
| 43387 | 154265405 | 154267236 | 121211 |
| 43388 | 154353859 | 154355373 | |
| 43389 | 154386580 | 154389011 | 121210 |
| 43390 | 154392308 | 154393429 | 121210 |
| 43391 | 154407106 | 154408430 | 121210 |
| 43392 | 154418266 | 154418867 | 121210 |
| 43393 | 154434004 | 154435706 | 121210 |
| 43394 | 154439005 | 154440721 | 121210 |
| 43395 | 154444260 | 154445634 | 121210 |
| 43396 | 154450623 | 154451475 | 121210 |
| 43397 | 154453525 | 154454145 | 121210 |
| 43398 | 154485392 | 154486796 | 121210 |
| 43399 | 154487012 | 154488695 | 121210 |
| 43400 | 154575964 | 154577667 | 250771 |
| 43401 | 154582633 | 154583080 | 250771 |
| 43402 | 154597751 | 154598645 | 250771 |
| 43403 | 154605068 | 154606075 | |
| 43404 | 154965170 | 154965856 | |
| 43405 | 155337802 | 155338500 | 197410 |
| 43406 | 155470744 | 155472392 | 171566 |
| 43407 | 155480434 | 155487531 | 171564 |
| 43408 | 155490032 | 155490946 | 171564 |
| 43409 | 155493910 | 155495488 | |
| 43410 | 155498132 | 155499506 | |
| 43411 | 155506370 | 155513558 | 171560 |
| 43412 | 155516880 | 155517570 | |
| 43413 | 155517845 | 155520050 | |
| 43414 | 155532118 | 155534803 | 171557 |
| 43415 | 155538542 | 155540364 | |
| 43416 | 155546936 | 155547510 | |
| 43417 | 155547833 | 155549103 | 121207 |
| 43418 | 155579888 | 155580627 | 121207 |
| 43419 | 155580719 | 155581922 | 121207 |
| 43420 | 155604908 | 155605743 | 121207 |
| 43421 | 156587839 | 156589101 | 164116 |
| 43422 | 156624314 | 156626082 | 164116 |
| 43423 | 156679807 | 156680658 | 061918 |
| 43424 | 156699154 | 156700286 | 061918 |
| 43425 | 156770230 | 156770726 | 256394 |
| 43426 | 156775410 | 156776694 | 151790, 256394 |
| 43427 | 156802492 | 156803541 | 151790 |
| 43428 | 156804867 | 156805511 | 151790 |
| 43429 | 156809856 | 156811308 | 151790 |
| 43430 | 156824557 | 156825087 | 151790 |
| 43431 | 156826362 | 156828987 | 151790 |
| 43432 | 156845396 | 156846275 | 256043 |
| 43433 | 156873210 | 156876882 | 256043 |
| 43434 | 157111017 | 157113603 | |
| 43435 | 157250573 | 157252131 | |
| 43436 | 157691739 | 157697499 | 145431 |
| 43437 | 157700882 | 157701401 | 145431 |
| 43438 | 157851816 | 157852901 | 145431 |
| 43439 | 157866555 | 157869244 | 145431 |
| 43440 | 157869335 | 157871985 | 145431 |
| 43441 | 157872045 | 157874057 | 145431 |
| 43442 | 157889403 | 157894177 | 250622, 145431 |
| 43443 | 157897078 | 157897834 | |
| 43444 | 157919711 | 157921280 | |
| 43445 | 157924364 | 157926681 | |
| 43446 | 159131014 | 159132923 | 164124 |
| 43447 | 159220529 | 159221047 | |
| 43448 | 159592354 | 159594419 | 171503, 205208 |
| 43449 | 159643244 | 159645310 | 171497 |
| 43450 | 159689068 | 159691203 | 052795 |
| 43451 | 159703455 | 159704864 | 052795 |
| 43452 | 159713546 | 159715076 | 052795 |
| 43453 | 159716013 | 159723495 | 052795 |
| 43454 | 159730546 | 159733223 | 052795 |
| 43455 | 159735306 | 159736128 | 052795 |
| 43456 | 159744331 | 159748936 | 052795 |
| 43457 | 159779118 | 159780456 | 052795 |
| 43458 | 159823748 | 159824290 | 164123, 052795 |
| 43459 | 159866717 | 159867358 | 164123 |
| 43460 | 159916011 | 159917303 | 164123 |
| 43461 | 159917581 | 159918893 | 164123 |
| 43462 | 159931190 | 159932818 | 164123 |
| 43463 | 159935859 | 159936585 | 164123 |
| 43464 | 159967862 | 159968932 | |
| 43465 | 159969051 | 159970011 | |
| 43466 | 159981194 | 159981665 | |
| 43467 | 159992356 | 159993371 | |
| 43468 | 160023272 | 160026585 | 109756 |
| 43469 | 160062267 | 160062832 | 109756 |
| 43470 | 160092421 | 160092831 | 109756 |
| 43471 | 160140858 | 160141803 | 109756 |
| 43472 | 160145472 | 160146127 | 109756 |
| 43473 | 160148878 | 160149492 | 109756 |
| 43474 | 160169690 | 160170660 | 109756 |
| 43475 | 160179690 | 160180969 | 109756 |
| 43476 | 160216653 | 160218822 | 216089, 109756 |
| 43477 | 160219134 | 160220088 | 109756 |
| 43478 | 160419547 | 160420693 | |
| 43479 | 160782023 | 160782611 | |
| 43480 | 162103870 | 162104687 | |
| 43481 | 163603645 | 163607052 | |
| 43482 | 164086913 | 164088753 | 145414 |
| 43483 | 164415001 | 164417048 | 198498 |
| 43484 | 165383018 | 165383966 | |
| 43485 | 166033170 | 166034764 | 170088 |
| 43486 | 166079691 | 166080526 | 170088 |
| 43487 | 166128042 | 166130002 | 109466, 170088 |
| 43488 | 166143135 | 166144122 | 109466 |
| 43489 | 166246222 | 166250770 | 052802 |
| 43490 | 166288218 | 166288581 | 109472 |
| 43491 | 166290601 | 166291148 | 109472 |
| 43492 | 166299919 | 166300715 | 109472 |
| 43493 | 166423494 | 166425005 | |
| 43494 | 166427086 | 166428012 | |
| 43495 | 167942431 | 167945753 | 196104 |
| 43496 | 167945839 | 167947788 | 196104 |
| 43497 | 167952033 | 167956674 | 196104 |
| 43498 | 168117410 | 168118474 | 196104 |
| 43499 | 169085479 | 169087281 | 109511 |
| 43500 | 169239029 | 169240388 | 137628 |
| 43501 | 169360878 | 169362683 | 181381 |
| 43502 | 169372071 | 169373498 | 181381 |
| 43503 | 169400865 | 169403523 | 181381 |
| 43504 | 169407531 | 169409615 | 181381 |
| 43505 | 169419336 | 169419818 | 181381, 129116 |
| 43506 | 169422397 | 169423122 | 181381, 129116 |
| 43507 | 169510031 | 169510948 | 129116 |
| 43508 | 169533433 | 169534367 | 129116 |
| 43509 | 169552558 | 169553508 | 129116 |
| 43510 | 169600512 | 169601341 | 129116 |
| 43511 | 169620212 | 169620968 | 129116 |
| 43512 | 169716475 | 169719566 | 129116 |
| 43513 | 169736827 | 169739163 | 129116 |
| 43514 | 169752378 | 169755302 | 249609, 129116 |
| 43515 | 169798751 | 169800056 | 129116, 145439 |
| 43516 | 169840688 | 169841493 | 129116, 145439 |
| 43517 | 169926280 | 169927452 | 201818, 145439 |
| 43518 | 169929399 | 169932262 | 251445, 145439 |
| 43519 | 169935424 | 169937591 | |
| 43520 | 169961603 | 169962143 | |
| 43521 | 169972832 | 169973516 | |
| 43522 | 169983411 | 169984189 | |
| 43523 | 170025052 | 170025712 | 154447 |
| 43524 | 170067640 | 170068820 | 154447 |
| 43525 | 170164756 | 170165384 | 154447 |
| 43526 | 170170170 | 170176406 | 154447 |
| 43527 | 170188097 | 170189417 | 154447 |
| 43528 | 170189510 | 170193268 | 154447 |
| 43529 | 170230379 | 170231042 | |
| 43530 | 170235424 | 170237174 | |
| 43531 | 170338170 | 170339528 | 137601 |
| 43532 | 170358918 | 170359904 | 137601 |
| 43533 | 170532543 | 170534363 | 109572, 137601 |
| 43534 | 170540583 | 170542908 | 109572 |
| 43535 | 170571265 | 170572063 | 109572 |
| 43536 | 170580937 | 170581935 | 109572 |
| 43537 | 170662642 | 170663067 | 056050 |
| 43538 | 170678753 | 170679314 | 056050 |
| 43539 | 170827063 | 170828124 | |
| 43540 | 170828731 | 170829229 | |
| 43541 | 170836592 | 170837121 | |
| 43542 | 170889503 | 170891444 | 248319 |
| 43543 | 170896190 | 170897639 | 248319 |
| 43544 | 170946568 | 170949110 | 198948 |
| 43545 | 171010383 | 171011784 | 109576 |
| 43546 | 173645667 | 173648004 | 174473 |
| 43547 | 173903074 | 173904482 | 174473 |
| 43548 | 174254525 | 174256672 | 164104 |
| 43549 | 174290921 | 174292484 | 164105, 272870 |
| 43550 | 175181222 | 175182614 | 164117 |
| 43551 | 175204067 | 175206297 | 164118, 164117 |
| 43552 | 175381302 | 175383955 | |
| 43553 | 175411417 | 175412317 | 164120 |
| 43554 | 175442690 | 175444714 | 164120 |
| 43555 | 175459114 | 175459988 | 251584, 250596 |
| 43556 | 175476813 | 175478247 | |
| 43557 | 175788698 | 175789736 | 168594 |
| 43558 | 176191277 | 176192897 | |
| 43559 | 176194988 | 176196398 | |
| 43560 | 176458788 | 176459374 | |
| 43561 | 176460216 | 176463253 | |
| 43562 | 177240428 | 177242592 | 129128, 248980 |
| 43563 | 178332928 | 178333687 | |
| 43564 | 178362216 | 178364713 | 038002 |
| 43565 | 178608146 | 178608756 | 251416 |
| 43566 | 179243624 | 179244475 | |
| 43567 | 179331918 | 179332749 | |
| 43568 | 179334364 | 179335640 | |
| 43569 | 183133428 | 183134090 | 218336 |
| 43570 | 183145292 | 183145754 | 218336 |
| 43571 | 183267825 | 183268602 | 218336 |
| 43572 | 183369716 | 183370458 | 218336 |
| 43573 | 183727441 | 183728063 | |
| 43574 | 183818763 | 183820073 | 129187 |
| 43575 | 183837356 | 183839633 | 129187 |
| 43576 | 183843543 | 183844322 | |
| 43577 | 183995431 | 183996357 | |
| 43578 | 184001447 | 184002793 | |
| 43579 | 184015699 | 184016540 | 272784 |
| 43580 | 184018550 | 184021732 | 151718, 251359 |
| 43581 | 184023080 | 184023740 | 151718 |
| 43582 | 184028432 | 184029056 | 151718 |
| 43583 | 184031539 | 184033303 | 151718 |
| 43584 | 184039190 | 184040737 | 151718 |
| 43585 | 184070844 | 184071687 | 151718 |
| 43586 | 184075858 | 184076621 | 151718 |
| 43587 | 184091934 | 184093712 | 151718 |
| 43588 | 184181504 | 184182183 | 151718 |
| 43589 | 184254894 | 184256058 | |
| 43590 | 184319231 | 184320228 | |
| 43591 | 184364416 | 184367020 | 168564 |
| 43592 | 184389144 | 184390914 | |
| 43593 | 184424522 | 184427813 | 168556, 232648 |
| 43594 | 184492405 | 184493035 | |
| 43595 | 184538335 | 184540972 | |
| 43596 | 184557508 | 184559438 | |
| 43597 | 184579510 | 184581277 | 168538, 182552 |
| 43598 | 184621355 | 184621957 | 168538 |
| 43599 | 184642174 | 184643306 | |
| 43600 | 184643519 | 184644380 | |
| 43601 | 184826119 | 184828681 | 173320 |
| 43602 | 184848039 | 184848522 | 173320 |
| 43603 | 184861029 | 184861897 | 173320 |
| 43604 | 184909229 | 184909877 | 248206, 173320 |
| 43605 | 184985749 | 184986538 | |
| 43606 | 184987893 | 184988729 | |
| 43607 | 185025335 | 185026464 | 164303 |
| 43608 | 185088910 | 185090146 | 164303 |
| 43609 | 185123795 | 185124154 | 164303 |
| 43610 | 185153156 | 185154601 | |
| 43611 | 185185209 | 185185806 | |
| 43612 | 185208507 | 185209437 | |
| 43613 | 185268522 | 185270835 | 180712 |
| 43614 | 185302548 | 185306785 | 249096 |
| 43615 | 185321450 | 185322939 | 168310 |
| 43616 | 185338173 | 185339450 | 168310 |
| 43617 | 185353736 | 185355632 | 168310 |
| 43618 | 185366025 | 185366489 | 168310 |
| 43619 | 185392865 | 185396974 | 270426, 271646, 168310 |
| 43620 | 185400194 | 185401267 | |
| 43621 | 185457874 | 185460078 | 271538 |
| 43622 | 185462436 | 185464047 | |
| 43623 | 185468094 | 185469175 | |
| 43624 | 185568876 | 185571514 | 164306, 164305 |
| 43625 | 185587613 | 185588402 | 164306 |
| 43626 | 185654655 | 185655891 | 151725 |
| 43627 | 185666853 | 185669040 | |
| 43628 | 185696924 | 185697717 | 151726 |
| 43629 | 185713280 | 185716705 | 151726 |
| 43630 | 185722758 | 185725691 | 151726 |
| 43631 | 185726337 | 185727824 | 151726 |
| 43632 | 185728983 | 185729923 | 151726 |
| 43633 | 185732648 | 185735463 | 251139, 151726 |
| 43634 | 185735620 | 185737557 | 151726, 251139 |
| 43635 | 185740644 | 185741756 | 151726, 251139 |
| 43636 | 185745291 | 185748453 | 151726 |
| 43637 | 185805537 | 185806503 | |
| 43638 | 185817760 | 185821657 | 249173 |
| 43639 | 185881441 | 185882137 | |
| 43640 | 186029578 | 186030203 | |
| 43641 | 186033057 | 186033850 | |
| 43642 | 186036990 | 186037639 | |
| 43643 | 186063368 | 186064955 | 151729 |
| 43644 | 186124315 | 186125512 | 109762, 164323 |
| 43645 | 186316354 | 186318600 | 186352, 109771 |
| 43646 | 186346186 | 186347886 | 205129, 109775 |
| 43647 | 186392400 | 186392981 | 249679, 168491 |
| 43648 | 186424884 | 186426631 | 154553 |
| 43649 | 186428998 | 186429670 | 154553 |
| 43650 | 186434576 | 186435771 | 154553 |
| 43651 | 186512759 | 186513369 | 233110, 154556 |
| 43652 | 186572348 | 186572817 | 154556 |
| 43653 | 186602642 | 186605205 | 154556 |
| 43654 | 186620737 | 186622644 | 154556 |
| 43655 | 186628077 | 186635971 | 207497, 154556 |
| 43656 | 186639241 | 186644335 | 154556 |
| 43657 | 186658671 | 186660506 | 154556 |
| 43658 | 186662576 | 186664422 | 154556 |
| 43659 | 186693384 | 186697748 | 154556 |
| 43660 | 186721403 | 186722096 | 154556 |
| 43661 | 186729571 | 186735374 | 154556 |
| 43662 | 186738315 | 186739182 | 154556 |
| 43663 | 186751306 | 186755536 | 154556 |
| 43664 | 186757202 | 186759734 | 154556 |
| 43665 | 186760060 | 186761096 | 154556 |
| 43666 | 186786506 | 186787925 | 154556 |
| 43667 | 186795415 | 186796981 | 154556 |
| 43668 | 186823683 | 186824345 | 154556 |
| 43669 | 186950375 | 186951284 | |
| 43670 | 186988939 | 186991145 | 164342 |
| 43671 | 187025568 | 187026974 | 109794 |
| 43672 | 187064872 | 187066984 | 109794 |
| 43673 | 187082304 | 187082771 | 109794 |
| 43674 | 187087865 | 187088805 | 109794 |
| 43675 | 187111109 | 187114243 | 269302, 145476 |
| 43676 | 187147868 | 187150386 | 164344 |
| 43677 | 187180052 | 187184264 | |
| 43678 | 187186621 | 187189007 | 088926 |
| 43679 | 187564331 | 187566753 | 083857 |
| 43680 | 187587343 | 187589313 | 083857 |
| 43681 | 187606012 | 187607770 | 083857 |
| 43682 | 187644000 | 187651466 | 083857 |
| 43683 | 187654807 | 187658242 | |
| 43684 | 187676803 | 187677558 | |
| 43685 | 187686230 | 187687816 | |
| 43686 | 187691312 | 187692891 | |
| 43687 | 187711845 | 187712390 | |
| 43688 | 187726027 | 187727974 | |
| 43689 | 187734353 | 187735044 | |
| 43690 | 187750814 | 187751419 | |
| 43691 | 187821726 | 187822785 | 249539 |
| 43692 | 187822839 | 187826649 | |
| 43693 | 187831366 | 187832294 | |
| 43694 | 187857310 | 187858668 | |
| 43695 | 190861142 | 190862789 | 109536, 245685, 145332 |

The locations of ODSCs on Chromosome 5 are shown in Table 47.

TABLE 47
ODSC Locations on Chromosome 5

| SEQ ID NO. | Start | End | Overlapping Gene(s) |
|---|---|---|---|
| 43696 | 217722 | 219541 | 073578, 164366 |
| 43697 | 263509 | 264250 | |
| 43698 | 271008 | 272507 | 249915, 248925 |
| 43699 | 442207 | 444390 | 180104, 221990 |
| 43700 | 472050 | 474287 | 225138, 066230, 188242, |
| | | | 180104 |
| 43701 | 611711 | 613154 | 112877, 249650 |
| 43702 | 671373 | 672378 | 171368 |
| 43703 | 693299 | 693931 | 171368 |
| 43704 | 891753 | 893177 | 071539, 028310 |
| 43705 | 956830 | 958485 | |
| 43706 | 958573 | 959632 | |
| 43707 | 1004370 | 1005439 | 272347, 221244 |
| 43708 | 1089400 | 1090973 | 113504 |
| 43709 | 1098849 | 1099852 | 113504 |
| 43710 | 1101622 | 1104904 | 113504 |
| 43711 | 1105505 | 1112420 | 113504 |
| 43712 | 1119383 | 1120121 | |
| 43713 | 1129093 | 1130278 | |
| 43714 | 1140324 | 1141137 | |
| 43715 | 1174439 | 1174910 | 249201 |
| 43716 | 1181539 | 1182392 | |
| 43717 | 1182625 | 1183697 | |
| 43718 | 1185758 | 1186454 | |
| 43719 | 1315577 | 1316721 | |
| 43720 | 1337109 | 1337720 | 049656 |
| 43721 | 1344207 | 1346188 | 049656 |
| 43722 | 1385719 | 1386808 | |
| 43723 | 1515298 | 1518255 | 153395 |
| 43724 | 1522207 | 1525076 | 153395 |
| 43725 | 1535845 | 1537631 | |
| 43726 | 1568232 | 1568828 | 185986 |
| 43727 | 1594137 | 1595076 | 271119, 185986 |
| 43728 | 1633469 | 1634775 | 188002 |
| 43729 | 1799006 | 1802354 | 145494, 171421 |
| 43730 | 5063541 | 5064303 | 215231 |
| 43731 | 5135560 | 5136738 | 250579 |
| 43732 | 5363049 | 5363454 | |
| 43733 | 5421274 | 5423779 | 164151 |
| 43734 | 5795151 | 5795668 | |
| 43735 | 6158280 | 6161038 | |
| 43736 | 6162109 | 6163285 | |
| 43737 | 6274839 | 6275661 | |
| 43738 | 6280395 | 6281296 | |
| 43739 | 6377579 | 6378992 | 133398 |
| 43740 | 6470741 | 6471221 | 215218 |
| 43741 | 6472935 | 6473617 | 215218 |
| 43742 | 6495503 | 6497171 | |
| 43743 | 6508939 | 6509587 | |
| 43744 | 6583004 | 6583780 | 250056 |
| 43745 | 6590103 | 6591332 | |
| 43746 | 6591654 | 6594631 | |
| 43747 | 6595406 | 6596064 | |
| 43748 | 6632203 | 6635562 | 145545, 037474 |
| 43749 | 6636206 | 6637081 | 145545 |
| 43750 | 6659454 | 6661332 | 145545 |
| 43751 | 6711641 | 6713776 | |
| 43752 | 6714394 | 6715033 | 112941 |
| 43753 | 6736063 | 6738279 | 112941 |
| 43754 | 6759290 | 6760298 | |
| 43755 | 6765666 | 6766204 | 251365 |
| 43756 | 6766644 | 6767334 | 251365 |
| 43757 | 7851538 | 7852164 | 215217, 124275 |
| 43758 | 7868412 | 7870191 | 124279, 124275 |
| 43759 | 7877887 | 7879680 | 124275 |
| 43760 | 7887008 | 7887663 | 124275 |
| 43761 | 8456598 | 8458734 | 245729, 249159, 250037, |
| | | | 247516 |
| 43762 | 9059844 | 9060568 | 112902 |
| 43763 | 9197718 | 9198648 | 112902 |
| 43764 | 9376736 | 9377303 | 248537, 112902 |
| 43765 | 9380272 | 9380642 | 248537, 112902 |
| 43766 | 9389024 | 9389515 | 248537, 112902 |
| 43767 | 9402103 | 9402595 | 248537, 112902 |
| 43768 | 9408600 | 9410321 | 248537, 112902 |
| 43769 | 9523408 | 9524097 | 112902 |
| 43770 | 9546199 | 9547039 | 250786 |
| 43771 | 10249368 | 10251740 | 150753, 150756, 248968 |
| 43772 | 10263008 | 10264175 | 150753 |
| 43773 | 10265911 | 10268652 | 150753, 271980 |
| 43774 | 10272603 | 10273454 | |
| 43775 | 10288345 | 10290621 | 164237 |
| 43776 | 10306915 | 10308477 | 164237 |
| 43777 | 10310092 | 10311128 | |
| 43778 | 10331211 | 10332051 | |
| 43779 | 10333006 | 10334546 | |
| 43780 | 10352004 | 10355008 | 145495, 259802 |
| 43781 | 10460712 | 10461026 | 145491 |
| 43782 | 10461909 | 10462702 | 145491 |
| 43783 | 10550816 | 10553196 | |
| 43784 | 10563433 | 10564740 | 164236 |
| 43785 | 10595261 | 10596423 | 164236 |
| 43786 | 10601071 | 10602959 | 164236 |
| 43787 | 10611192 | 10611953 | 164236 |
| 43788 | 10627857 | 10628423 | 250106, 164236 |
| 43789 | 10663192 | 10664317 | |
| 43790 | 10671093 | 10672251 | |
| 43791 | 10691836 | 10692835 | 112977 |
| 43792 | 10701683 | 10703681 | 112977 |
| 43793 | 10712908 | 10713713 | 112977 |
| 43794 | 10719949 | 10720488 | 112977 |
| 43795 | 10733031 | 10733939 | 112977 |
| 43796 | 10746900 | 10749279 | 112977 |
| 43797 | 10759810 | 10762316 | 272324, 112977 |
| 43798 | 10833133 | 10833589 | |
| 43799 | 10962085 | 10962796 | |
| 43800 | 11324922 | 11325405 | 169862 |
| 43801 | 11733513 | 11734045 | 169862 |
| 43802 | 13566363 | 13568096 | |
| 43803 | 13640084 | 13640729 | |
| 43804 | 13695104 | 13695858 | 039139 |
| 43805 | 13910603 | 13911680 | 039139 |
| 43806 | 13985648 | 13986909 | |
| 43807 | 14008582 | 14010088 | |
| 43808 | 14010902 | 14012710 | |
| 43809 | 14034802 | 14040305 | |
| 43810 | 14105140 | 14111465 | |
| 43811 | 14141664 | 14150037 | 038382 |
| 43812 | 14156687 | 14157645 | 038382 |
| 43813 | 14165345 | 14166202 | 038382 |
| 43814 | 14203749 | 14206081 | 038382 |
| 43815 | 14207539 | 14210749 | 038382 |
| 43816 | 14212359 | 14214674 | 038382 |
| 43817 | 14217079 | 14218248 | 038382 |
| 43818 | 14233789 | 14234956 | 038382 |
| 43819 | 14263507 | 14272185 | 038382 |
| 43820 | 14569998 | 14570745 | |
| 43821 | 14660038 | 14660774 | |
| 43822 | 14663415 | 14666900 | 154124, 261360 |
| 43823 | 14667422 | 14669225 | 154124 |
| 43824 | 14669967 | 14673190 | 154124 |
| 43825 | 14684253 | 14686279 | 154124 |
| 43826 | 14727369 | 14729168 | 154122 |
| 43827 | 14760386 | 14761413 | 154122 |
| 43828 | 14809235 | 14811806 | 154122 |
| 43829 | 14816343 | 14818236 | 154122 |
| 43830 | 14824032 | 14825500 | 154122 |
| 43831 | 14829711 | 14831191 | 154122 |
| 43832 | 14870899 | 14873682 | 272057, 154122 |
| 43833 | 15008210 | 15010656 | |
| 43834 | 15014717 | 15015491 | |
| 43835 | 15018536 | 15019184 | |
| 43836 | 15031232 | 15031937 | |
| 43837 | 15366588 | 15367702 | |
| 43838 | 15376723 | 15377421 | |
| 43839 | 15474038 | 15474932 | |
| 43840 | 15499679 | 15501024 | 183580 |
| 43841 | 15510776 | 15511413 | 183580 |
| 43842 | 15992362 | 15994040 | |
| 43843 | 16454733 | 16457110 | 173545 |
| 43844 | 16461858 | 16462317 | 173545 |
| 43845 | 16464769 | 16468863 | 173545 |
| 43846 | 16507365 | 16510282 | 154153 |
| 43847 | 16520659 | 16522781 | 154153 |
| 43848 | 16538095 | 16541765 | 154153 |
| 43849 | 16546562 | 16547674 | 154153 |
| 43850 | 16585145 | 16585744 | 154153 |
| 43851 | 16588141 | 16589441 | 154153 |
| 43852 | 16593001 | 16594226 | 154153 |
| 43853 | 18179204 | 18180175 | |
| 43854 | 18745274 | 18746870 | 248693 |
| 43855 | 18792111 | 18792640 | |
| 43856 | 21152653 | 21153367 | |
| 43857 | 21813838 | 21814644 | 154162 |
| 43858 | 23012589 | 23014120 | 249404 |
| 43859 | 23461573 | 23462389 | |
| 43860 | 23466202 | 23467410 | |
| 43861 | 23631251 | 23631767 | |
| 43862 | 24510159 | 24511428 | 040731 |
| 43863 | 25881475 | 25882171 | |
| 43864 | 25963642 | 25965731 | |
| 43865 | 25970896 | 25971633 | |
| 43866 | 26388437 | 26389581 | 249099 |
| 43867 | 30504328 | 30506616 | |
| 43868 | 30538913 | 30539922 | |
| 43869 | 30605605 | 30606323 | |
| 43870 | 31027042 | 31029043 | |
| 43871 | 31532020 | 31533190 | 082213, 113360 |
| 43872 | 31604660 | 31605400 | |
| 43873 | 32067800 | 32068732 | 133401 |
| 43874 | 32093206 | 32093798 | 133401 |
| 43875 | 32172463 | 32175724 | 272086, 113384 |
| 43876 | 32193496 | 32194210 | |
| 43877 | 32200171 | 32202953 | |
| 43878 | 32210919 | 32211825 | |
| 43879 | 32224038 | 32224669 | |
| 43880 | 32225439 | 32226160 | |
| 43881 | 32232337 | 32234749 | 150712 |
| 43882 | 32249640 | 32250331 | 150712 |
| 43883 | 32302769 | 32303316 | 150712 |
| 43884 | 32312033 | 32313786 | 150712 |
| 43885 | 32443169 | 32445117 | 056097 |
| 43886 | 32531443 | 32532812 | 113387 |
| 43887 | 32566336 | 32567160 | 113387 |
| 43888 | 32574359 | 32576175 | 113387 |
| 43889 | 32584943 | 32587092 | 113387 |
| 43890 | 32599840 | 32600580 | 113387 |
| 43891 | 32627726 | 32629288 | |
| 43892 | 32630758 | 32631994 | |
| 43893 | 32639999 | 32641153 | |
| 43894 | 32642200 | 32643428 | |
| 43895 | 32686929 | 32688405 | |
| 43896 | 32709592 | 32711588 | 113389 |
| 43897 | 32766856 | 32769684 | 113389 |
| 43898 | 32935344 | 32935931 | 250697 |
| 43899 | 33146317 | 33149286 | 250697 |
| 43900 | 33205130 | 33206662 | 250697 |
| 43901 | 33223439 | 33224649 | 250697 |
| 43902 | 33229196 | 33229932 | 251443, 250697 |
| 43903 | 33439695 | 33442559 | 113407, 249572 |
| 43904 | 33582398 | 33582976 | 151388 |
| 43905 | 34001292 | 34002096 | 273294, 242110 |
| 43906 | 34006935 | 34009790 | 242110, 273294 |
| 43907 | 34025908 | 34026993 | 273294, 082196 |
| 43908 | 34039448 | 34041704 | 273294, 082196 |
| 43909 | 34045154 | 34047110 | 273294 |
| 43910 | 34062075 | 34062497 | 273294 |
| 43911 | 34483996 | 34490045 | |
| 43912 | 34498431 | 34500279 | |
| 43913 | 34500282 | 34504490 | |
| 43914 | 34511132 | 34511554 | |
| 43915 | 34519155 | 34520335 | |
| 43916 | 34525261 | 34526578 | |
| 43917 | 34585057 | 34589857 | |
| 43918 | 34598157 | 34600094 | |
| 43919 | 34655199 | 34658151 | 250234, 039560 |
| 43920 | 34685681 | 34687784 | 039560 |
| 43921 | 34721380 | 34721964 | 039560 |
| 43922 | 34731363 | 34732524 | 039560 |
| 43923 | 34736526 | 34737330 | 039560 |
| 43924 | 34756786 | 34757700 | 039560 |
| 43925 | 34838777 | 34840059 | 205838, 272323 |
| 43926 | 34914736 | 34916710 | 113460, 113456 |
| 43927 | 34929226 | 34931322 | 168724 |
| 43928 | 34957224 | 34957966 | 168724 |
| 43929 | 35026233 | 35026905 | 113492 |
| 43930 | 35046865 | 35048886 | 113494, 113492 |
| 43931 | 35050393 | 35051456 | 269027, 113494 |
| 43932 | 35059896 | 35061682 | 113494 |
| 43933 | 35062815 | 35064009 | 113494 |
| 43934 | 35081226 | 35081665 | 250886, 113494 |
| 43935 | 35088136 | 35088669 | 113494 |
| 43936 | 35129364 | 35130606 | 113494 |
| 43937 | 35197699 | 35198666 | 113494 |
| 43938 | 35229191 | 35230971 | 113494 |
| 43939 | 35237680 | 35238396 | |
| 43940 | 35416307 | 35417333 | |
| 43941 | 35457885 | 35459589 | |
| 43942 | 35479014 | 35480536 | |
| 43943 | 35617529 | 35618676 | 152582 |
| 43944 | 35741049 | 35741835 | 248969, 152582 |
| 43945 | 35990365 | 35991989 | 145626 |
| 43946 | 35999210 | 36000432 | 145626 |
| 43947 | 36151072 | 36152433 | 145604, 164187 |
| 43948 | 36180108 | 36181987 | 145604 |
| 43949 | 36207101 | 36207453 | 152620 |
| 43950 | 36227878 | 36228545 | 152620 |
| 43951 | 36239317 | 36240400 | 152620 |
| 43952 | 36240655 | 36244185 | 152620 |
| 43953 | 36271998 | 36272892 | 164188 |
| 43954 | 36305537 | 36306443 | |
| 43955 | 36324176 | 36325102 | |
| 43956 | 36363057 | 36363790 | |
| 43957 | 36373463 | 36374634 | |
| 43958 | 36403056 | 36404076 | |
| 43959 | 36417728 | 36418937 | |
| 43960 | 36441860 | 36443309 | |
| 43961 | 36528797 | 36529175 | |
| 43962 | 36657572 | 36658703 | 079215 |
| 43963 | 36739405 | 36741707 | |
| 43964 | 36743942 | 36745447 | |
| 43965 | 36875895 | 36878302 | 164190 |
| 43966 | 37064420 | 37065143 | 164190 |
| 43967 | 37248771 | 37250033 | 197603 |
| 43968 | 37370345 | 37371957 | 113569 |
| 43969 | 37494167 | 37495937 | 082068 |
| 43970 | 37662505 | 37663330 | 082068 |
| 43971 | 37667279 | 37670099 | 082068 |
| 43972 | 37695491 | 37697260 | 082068 |
| 43973 | 37717992 | 37718611 | 082068 |
| 43974 | 37722308 | 37723984 | 082068 |
| 43975 | 37769227 | 37770394 | |
| 43976 | 37799852 | 37800771 | |
| 43977 | 37839601 | 37840984 | 248587, 168621 |
| 43978 | 37847029 | 37849370 | 248587 |
| 43979 | 38069338 | 38070468 | 250003 |
| 43980 | 38463692 | 38465004 | 164318, 251257 |
| 43981 | 38556843 | 38557554 | 244968, 113594 |
| 43982 | 38830535 | 38831080 | 249740 |
| 43983 | 38845271 | 38847382 | 145623, 249740 |
| 43984 | 38871051 | 38873697 | 145623 |
| 43985 | 39013642 | 39017189 | 164327 |
| 43986 | 39072976 | 39077537 | 164327 |
| 43987 | 39100895 | 39103501 | |
| 43988 | 39255363 | 39258516 | 082074 |
| 43989 | 39353622 | 39354508 | 113600 |
| 43990 | 39360027 | 39364181 | 113600 |
| 43991 | 39368164 | 39370188 | 113600 |
| 43992 | 39370455 | 39371807 | 153071, 113600 |
| 43993 | 39373776 | 39374926 | 153071, 113600 |
| 43994 | 39398943 | 39401259 | 153071, 113600 |
| 43995 | 39424296 | 39425476 | 113600, 153071 |
| 43996 | 39436973 | 39438294 | 153071 |
| 43997 | 39624440 | 39625984 | |
| 43998 | 39638994 | 39640516 | |
| 43999 | 39648669 | 39649363 | |
| 44000 | 40547760 | 40549080 | |
| 44001 | 40679407 | 40680274 | 171522 |
| 44002 | 40717326 | 40717912 | 113638 |
| 44003 | 40733840 | 40736953 | 113638 |
| 44004 | 40755091 | 40756356 | 113638 |
| 44005 | 40783973 | 40784951 | 132356 |
| 44006 | 40797306 | 40799147 | 132356 |
| 44007 | 40833860 | 40836971 | 145592 |
| 44008 | 40988620 | 40992639 | |
| 44009 | 41156793 | 41157359 | 039537 |
| 44010 | 41193572 | 41195841 | 039537 |
| 44011 | 41201306 | 41201920 | 039537 |
| 44012 | 41638540 | 41639520 | |
| 44013 | 41903796 | 41905551 | 205765 |
| 44014 | 41924668 | 41926310 | 151876 |
| 44015 | 42108345 | 42109234 | |
| 44016 | 42423104 | 42425392 | 112964 |
| 44017 | 42500270 | 42500927 | 112964 |
| 44018 | 42502945 | 42503637 | 112964 |
| 44019 | 42515593 | 42517842 | 112964 |
| 44020 | 42543317 | 42546016 | 112964 |
| 44021 | 42547172 | 42558476 | 112964 |
| 44022 | 42561301 | 42562725 | 112964 |
| 44023 | 42570282 | 42574072 | 112964 |
| 44024 | 42579346 | 42579802 | 112964 |
| 44025 | 42582674 | 42584339 | 112964 |
| 44026 | 42590631 | 42592503 | 112964 |
| 44027 | 42596299 | 42599660 | 112964 |
| 44028 | 42606230 | 42607170 | 112964 |
| 44029 | 42614370 | 42615031 | 112964 |
| 44030 | 42620535 | 42621299 | 112964 |
| 44031 | 42641101 | 42642145 | 112964 |
| 44032 | 42644918 | 42646225 | 112964 |
| 44033 | 42655403 | 42657176 | 112964 |
| 44034 | 42689387 | 42689894 | 112964 |
| 44035 | 42807964 | 42808656 | 250722 |
| 44036 | 42810829 | 42813501 | 250722 |
| 44037 | 42840331 | 42840951 | 250722 |
| 44038 | 42854631 | 42856468 | 250722 |
| 44039 | 42874496 | 42875598 | 250722 |
| 44040 | 42950751 | 42951703 | |
| 44041 | 43041509 | 43043479 | 215068, 177721, 177738 |
| 44042 | 43064849 | 43068180 | 172262, 177738 |
| 44043 | 43105321 | 43106597 | 172262 |
| 44044 | 43120329 | 43122687 | 172262 |
| 44045 | 43312405 | 43315797 | 112972 |
| 44046 | 43396661 | 43397594 | 151882 |
| 44047 | 43421716 | 43423052 | |
| 44048 | 43425578 | 43429564 | |
| 44049 | 43432399 | 43433136 | |
| 44050 | 43482501 | 43485451 | 249492, 151881 |
| 44051 | 43514922 | 43516199 | 248240, 172244, 248554 |
| 44052 | 43555902 | 43557957 | 172239 |
| 44053 | 43581882 | 43582991 | 248092 |
| 44054 | 43601479 | 43604678 | 112992, 248092 |
| 44055 | 43615422 | 43616314 | 112992 |
| 44056 | 43616638 | 43617030 | 112992 |
| 44057 | 43619314 | 43621084 | 112992 |
| 44058 | 43691109 | 43691857 | 112992 |
| 44059 | 43772961 | 43774016 | |
| 44060 | 43973241 | 43973934 | |
| 44061 | 43997416 | 43998543 | |
| 44062 | 44000840 | 44003200 | |
| 44063 | 44808559 | 44810719 | 112996, 251141 |
| 44064 | 45491480 | 45491924 | 164588 |
| 44065 | 49962076 | 49964192 | 151883 |
| 44066 | 50013432 | 50013867 | 151883 |
| 44067 | 50521489 | 50522545 | |
| 44068 | 51320039 | 51320877 | 248789 |
| 44069 | 52079106 | 52079655 | 248898 |
| 44070 | 52082116 | 52085227 | 152684, 213949, 248898 |
| 44071 | 52090311 | 52090986 | 152684, 213949 |
| 44072 | 52094703 | 52097712 | 152684, 213949 |
| 44073 | 52120122 | 52120736 | 213949 |
| 44074 | 52165968 | 52167166 | 213949 |
| 44075 | 52184132 | 52184897 | 213949 |
| 44076 | 52187998 | 52189699 | 213949 |
| 44077 | 52210743 | 52211673 | 213949 |
| 44078 | 52218116 | 52218787 | 213949 |
| 44079 | 52282635 | 52286368 | 164171, 249899 |
| 44080 | 52325518 | 52328790 | 164171 |
| 44081 | 52404817 | 52406501 | 247796, 164172 |
| 44082 | 52460252 | 52461106 | |
| 44083 | 52728805 | 52729624 | |
| 44084 | 52770989 | 52779562 | 134363 |
| 44085 | 52785693 | 52787725 | |
| 44086 | 52856083 | 52857232 | 164258 |
| 44087 | 53303898 | 53304694 | 185305 |
| 44088 | 53340873 | 53342033 | 185305 |
| 44089 | 53515967 | 53517388 | 185305 |
| 44090 | 53605461 | 53607089 | 185305 |
| 44091 | 53812874 | 53814983 | 178996 |
| 44092 | 53843185 | 53844311 | |
| 44093 | 53863664 | 53864322 | |
| 44094 | 53911783 | 53912319 | |
| 44095 | 53915259 | 53917505 | |
| 44096 | 53923480 | 53924707 | |
| 44097 | 53946829 | 53948374 | |
| 44098 | 53959575 | 53960135 | |
| 44099 | 54038163 | 54038951 | |
| 44100 | 54056843 | 54057610 | |
| 44101 | 54130143 | 54130767 | 232517 |
| 44102 | 54133478 | 54135256 | 232517 |
| 44103 | 54439133 | 54440061 | 164287 |
| 44104 | 54522723 | 54523662 | 234602 |
| 44105 | 54602375 | 54604701 | 039123, 067248 |
| 44106 | 54686189 | 54686731 | 039123 |
| 44107 | 54733269 | 54735394 | 067113 |
| 44108 | 54766999 | 54767753 | 067113 |
| 44109 | 54829853 | 54831824 | 067113 |
| 44110 | 54873107 | 54873740 | |
| 44111 | 54892471 | 54894287 | |
| 44112 | 54894886 | 54898111 | |
| 44113 | 54899767 | 54901053 | |
| 44114 | 54915820 | 54916529 | |
| 44115 | 54924992 | 54926396 | 177058 |
| 44116 | 54928221 | 54933555 | 177058 |
| 44117 | 54986885 | 54988153 | 177058 |
| 44118 | 55007173 | 55008970 | 177058 |
| 44119 | 55163770 | 55165142 | 164509 |
| 44120 | 55195038 | 55195638 | 164509 |
| 44121 | 55227208 | 55228544 | |
| 44122 | 55228840 | 55231261 | 134352 |
| 44123 | 55288581 | 55294517 | 227908, 134352 |
| 44124 | 55298231 | 55300087 | 227908 |
| 44125 | 55320387 | 55321987 | |
| 44126 | 55324938 | 55326373 | |
| 44127 | 55339279 | 55340107 | |
| 44128 | 55354037 | 55355405 | 249236 |
| 44129 | 55384866 | 55385694 | |
| 44130 | 55392743 | 55394698 | |
| 44131 | 55412299 | 55413152 | 164512 |
| 44132 | 55418793 | 55420328 | 164512 |
| 44133 | 55438331 | 55439011 | 164512 |
| 44134 | 55496335 | 55497528 | 164512 |
| 44135 | 55498226 | 55498758 | 164512 |
| 44136 | 55506880 | 55508360 | 164512 |
| 44137 | 55575141 | 55576825 | |
| 44138 | 55596669 | 55597278 | |
| 44139 | 55620552 | 55621341 | |
| 44140 | 55623548 | 55626192 | |
| 44141 | 55646112 | 55647005 | |
| 44142 | 55651368 | 55652708 | |
| 44143 | 55653933 | 55655909 | |
| 44144 | 55776398 | 55777340 | 248727 |
| 44145 | 55803101 | 55803835 | |
| 44146 | 55818580 | 55819490 | 225940 |
| 44147 | 55932196 | 55934826 | |
| 44148 | 56109986 | 56113042 | 095015 |
| 44149 | 56154587 | 56155330 | 237705, 095015 |
| 44150 | 56204690 | 56206763 | 155542, 225230 |
| 44151 | 56213288 | 56214252 | 155542 |
| 44152 | 56246174 | 56246777 | 155545 |
| 44153 | 56246817 | 56248618 | 155545 |
| 44154 | 56327897 | 56328502 | |
| 44155 | 56328956 | 56329372 | |
| 44156 | 56398037 | 56399616 | |
| 44157 | 56468180 | 56471760 | 062194 |
| 44158 | 56505167 | 56505974 | 062194 |
| 44159 | 56515165 | 56515660 | 062194 |
| 44160 | 56593895 | 56595210 | |
| 44161 | 56597903 | 56598510 | |
| 44162 | 56623051 | 56624744 | |
| 44163 | 56630401 | 56633270 | |
| 44164 | 56703505 | 56705156 | 250961 |
| 44165 | 56707753 | 56708465 | 250961 |
| 44166 | 56731131 | 56732588 | 250961 |
| 44167 | 56790218 | 56791381 | 250961 |
| 44168 | 56945996 | 56950099 | |
| 44169 | 56980279 | 56981346 | |
| 44170 | 56988067 | 56995621 | |
| 44171 | 57011055 | 57015240 | |
| 44172 | 57030081 | 57034604 | |
| 44173 | 57070981 | 57076003 | |
| 44174 | 57111148 | 57113772 | |
| 44175 | 57130550 | 57132330 | |
| 44176 | 57132404 | 57135609 | |
| 44177 | 57335812 | 57336316 | |
| 44178 | 57479155 | 57488044 | |
| 44179 | 57553728 | 57556566 | |
| 44180 | 57742271 | 57758989 | 145632 |
| 44181 | 57841595 | 57842460 | 250127 |
| 44182 | 57878372 | 57879136 | 152932 |
| 44183 | 57925490 | 57926293 | 152932 |
| 44184 | 58055414 | 58056189 | 152932, 248475 |
| 44185 | 58145705 | 58146597 | 152932, 248733 |
| 44186 | 58334897 | 58336044 | 247345, 113448 |
| 44187 | 58423466 | 58423906 | 113448 |
| 44188 | 58592091 | 58593979 | 113448 |
| 44189 | 58711609 | 58712865 | 113448 |
| 44190 | 58718038 | 58718828 | 113448 |
| 44191 | 58725783 | 58726695 | 113448 |
| 44192 | 58728976 | 58729437 | 113448 |
| 44193 | 58742223 | 58742700 | 113448 |
| 44194 | 58762089 | 58762570 | 113448 |
| 44195 | 58778834 | 58780993 | 113448 |
| 44196 | 58781118 | 58781903 | 113448 |
| 44197 | 58838771 | 58840357 | 113448 |
| 44198 | 58844475 | 58845565 | 113448 |
| 44199 | 58858657 | 58859319 | 113448 |
| 44200 | 58878832 | 58879636 | 113448 |
| 44201 | 58957603 | 58958521 | 113448 |
| 44202 | 59051864 | 59053004 | 113448 |
| 44203 | 59055186 | 59056207 | 113448 |
| 44204 | 59062016 | 59062723 | 113448 |
| 44205 | 59063492 | 59065104 | 113448 |
| 44206 | 59088882 | 59089341 | 113448 |
| 44207 | 59968442 | 59969146 | 035499 |
| 44208 | 59995368 | 59997121 | 035499 |
| 44209 | 60106324 | 60107011 | 164181 |
| 44210 | 60240092 | 60242111 | 164182, 049167 |
| 44211 | 60457297 | 60459797 | 251279, 188725 |
| 44212 | 60596923 | 60598823 | |
| 44213 | 60609812 | 60610319 | |
| 44214 | 60623917 | 60625243 | |
| 44215 | 60625753 | 60628521 | 130449 |
| 44216 | 60628569 | 60630401 | 130449 |
| 44217 | 60642817 | 60643383 | 251682, 130449 |
| 44218 | 60704138 | 60706110 | 130449 |
| 44219 | 60895495 | 60896109 | |
| 44220 | 60921247 | 60922321 | |
| 44221 | 60963249 | 60963911 | 178722, 248529 |
| 44222 | 61031619 | 61032100 | 178722 |
| 44223 | 61086354 | 61087350 | |
| 44224 | 61212945 | 61213347 | |
| 44225 | 61215567 | 61216293 | |
| 44226 | 61601329 | 61603462 | 068796 |
| 44227 | 61698734 | 61700287 | 086200, 086189, 068796 |
| 44228 | 61708310 | 61709252 | 086200, 068796 |
| 44229 | 61884435 | 61885044 | 086200 |
| 44230 | 62037651 | 62038674 | |
| 44231 | 62233216 | 62235142 | |
| 44232 | 62348575 | 62349841 | |
| 44233 | 62351165 | 62351865 | |
| 44234 | 62426804 | 62428310 | |
| 44235 | 62449539 | 62451606 | |
| 44236 | 62460217 | 62461757 | |
| 44237 | 62538811 | 62539460 | |
| 44238 | 62557131 | 62559257 | |
| 44239 | 63461027 | 63462413 | 164197 |
| 44240 | 63506500 | 63507303 | 164197 |
| 44241 | 64061712 | 64062362 | 153006 |
| 44242 | 64063410 | 64065515 | 153015, 153006 |
| 44243 | 64235521 | 64236428 | 153015 |
| 44244 | 64330033 | 64332397 | |
| 44245 | 64398187 | 64399721 | |
| 44246 | 64407774 | 64408344 | |
| 44247 | 64660571 | 64661315 | 049192 |
| 44248 | 64689732 | 64690419 | 049192 |
| 44249 | 64775615 | 64776784 | 049192 |
| 44250 | 64777367 | 64780052 | 049192 |
| 44251 | 64858526 | 64860007 | 113593, 123219 |
| 44252 | 64906933 | 64908695 | 113595 |
| 44253 | 64919334 | 64922001 | 113597, 253251, 113595 |
| 44254 | 65017819 | 65019378 | 123213, 197860 |
| 44255 | 65220490 | 65223895 | 112851, 269961 |
| 44256 | 65439063 | 65441816 | 253744, 153914 |
| 44257 | 65543034 | 65543442 | |
| 44258 | 65891612 | 65892678 | 069020 |
| 44259 | 65910753 | 65911349 | 069020 |
| 44260 | 65985034 | 65987350 | 069020 |
| 44261 | 66023057 | 66024104 | 069020 |
| 44262 | 66037056 | 66037649 | 069020 |
| 44263 | 66144723 | 66146165 | 069020 |
| 44264 | 66180410 | 66181143 | 069020 |
| 44265 | 66186554 | 66188768 | 069020 |
| 44266 | 66254816 | 66256060 | 069020 |
| 44267 | 66256194 | 66256684 | 069020 |
| 44268 | 66259013 | 66260052 | 069020 |
| 44269 | 66263389 | 66264384 | 069020 |
| 44270 | 66283667 | 66284304 | 069020 |
| 44271 | 66299029 | 66301649 | 229666, 069020 |
| 44272 | 66310203 | 66312126 | 069020 |
| 44273 | 66339429 | 66340352 | 069020 |
| 44274 | 66381308 | 66381887 | 069020 |
| 44275 | 66390236 | 66391817 | 069020 |
| 44276 | 66510551 | 66512484 | |
| 44277 | 66530085 | 66532218 | |
| 44278 | 66562687 | 66565747 | 251206 |
| 44279 | 66668791 | 66670222 | |
| 44280 | 66844104 | 66845432 | 249364 |
| 44281 | 66876284 | 66877735 | 249364 |
| 44282 | 66901613 | 66902966 | 249364 |
| 44283 | 67047663 | 67048458 | 250421, 249364 |
| 44284 | 67065807 | 67067609 | 250421, 249364 |
| 44285 | 67088528 | 67092543 | 250421, 249364 |
| 44286 | 67095544 | 67096130 | 250421, 249364 |
| 44287 | 67110332 | 67113876 | 250421, 249894 |
| 44288 | 67266988 | 67268204 | |
| 44289 | 67293850 | 67295729 | |
| 44290 | 67342185 | 67342773 | |
| 44291 | 67376159 | 67376894 | |
| 44292 | 67429210 | 67431605 | |
| 44293 | 67435752 | 67438159 | |
| 44294 | 67462640 | 67463600 | |
| 44295 | 67465108 | 67468657 | |
| 44296 | 67509981 | 67513258 | 145675 |
| 44297 | 67515041 | 67515733 | 145675 |
| 44298 | 67520824 | 67522286 | 145675 |
| 44299 | 67535061 | 67536187 | 145675 |
| 44300 | 67559256 | 67561991 | 145675 |
| 44301 | 67581911 | 67586910 | 145675 |
| 44302 | 67614386 | 67617222 | |
| 44303 | 67628567 | 67630541 | |
| 44304 | 67638609 | 67641091 | |
| 44305 | 67672817 | 67673252 | |
| 44306 | 67691462 | 67693199 | |
| 44307 | 67700858 | 67701262 | |
| 44308 | 67715340 | 67716133 | |
| 44309 | 67744969 | 67745857 | |
| 44310 | 67752022 | 67753066 | |
| 44311 | 67801254 | 67802588 | |
| 44312 | 67829709 | 67831245 | |
| 44313 | 67853002 | 67853928 | |
| 44314 | 67858083 | 67858602 | |
| 44315 | 67888835 | 67890169 | |
| 44316 | 67968670 | 67970130 | |
| 44317 | 67976295 | 67976874 | |
| 44318 | 67977657 | 67980323 | |
| 44319 | 68025487 | 68027701 | |
| 44320 | 68035875 | 68037159 | |
| 44321 | 68039406 | 68043588 | |
| 44322 | 68048868 | 68049541 | |
| 44323 | 68053358 | 68054480 | |
| 44324 | 68220042 | 68220797 | 249335 |
| 44325 | 68343957 | 68345658 | |
| 44326 | 68374732 | 68376184 | |
| 44327 | 68389449 | 68390978 | 145740 |
| 44328 | 68462335 | 68463047 | 134057 |
| 44329 | 68484742 | 68485981 | 153044 |
| 44330 | 68502310 | 68503012 | 153044 |
| 44331 | 68513365 | 68514375 | 134056 |
| 44332 | 68530185 | 68531588 | 134058 |
| 44333 | 68534673 | 68535015 | 134058 |
| 44334 | 68627383 | 68629325 | 183323 |
| 44335 | 68663953 | 68666430 | 152942, 085231 |
| 44336 | 68710534 | 68712267 | 152939, 152942 |
| 44337 | 68739346 | 68741085 | 152939 |
| 44338 | 68747011 | 68748802 | |
| 44339 | 68749843 | 68751261 | |
| 44340 | 68787561 | 68790255 | 197822, 253203, 249295 |
| 44341 | 68796874 | 68799847 | 249295, 197822, 253203 |
| 44342 | 68812246 | 68814823 | 197822, 253203 |
| 44343 | 70751151 | 70752603 | 145734 |
| 44344 | 70777899 | 70780509 | 145734 |
| 44345 | 70882420 | 70884068 | 131844 |
| 44346 | 71146563 | 71147125 | |
| 44347 | 71218328 | 71219353 | |
| 44348 | 71226925 | 71230016 | |
| 44349 | 71603048 | 71604731 | 113048 |
| 44350 | 71615281 | 71616873 | 049883, 113048 |
| 44351 | 71641938 | 71642736 | 049883 |
| 44352 | 71658403 | 71659744 | 272093 |
| 44353 | 71683840 | 71684415 | |
| 44354 | 71748124 | 71749299 | 178175 |
| 44355 | 72111368 | 72113971 | 083312, 249085 |
| 44356 | 72143671 | 72145256 | 083312 |
| 44357 | 72233780 | 72234594 | |
| 44358 | 72250465 | 72253212 | 157107, 272081 |
| 44359 | 72414581 | 72417272 | 157111 |
| 44360 | 72511712 | 72512413 | 249743 |
| 44361 | 72793395 | 72795502 | 145741, 272525 |
| 44362 | 72860306 | 72862621 | 164338, 164331 |
| 44363 | 72910905 | 72911356 | |
| 44364 | 72921028 | 72923366 | 214944 |
| 44365 | 72949721 | 72951463 | 214944 |
| 44366 | 72968378 | 72970731 | 214944 |
| 44367 | 72973754 | 72975094 | 214944 |
| 44368 | 73024264 | 73025601 | 214944 |
| 44369 | 73058658 | 73061373 | 214944 |
| 44370 | 73091862 | 73093218 | 214944 |
| 44371 | 73118763 | 73119980 | 214944 |
| 44372 | 73157345 | 73157794 | 214944 |
| 44373 | 73197033 | 73197894 | 214944 |
| 44374 | 73213155 | 73213577 | 214944 |
| 44375 | 73584265 | 73585589 | |
| 44376 | 73611148 | 73612979 | |
| 44377 | 73720081 | 73720740 | 248673 |
| 44378 | 73789148 | 73789842 | 248673 |
| 44379 | 73795395 | 73796822 | 248673 |
| 44380 | 73833435 | 73834612 | |
| 44381 | 73871504 | 73873488 | |
| 44382 | 73876606 | 73877828 | |
| 44383 | 73890187 | 73891890 | |
| 44384 | 73894730 | 73895903 | |
| 44385 | 73927791 | 73928482 | 171617 |
| 44386 | 73935537 | 73938676 | 049860, 171617 |
| 44387 | 73980735 | 73982049 | 049860 |
| 44388 | 73983204 | 73983546 | 049860 |
| 44389 | 74056979 | 74058003 | 164347 |
| 44390 | 74058376 | 74059315 | 164347 |
| 44391 | 74062032 | 74063830 | 164346, 164347 |
| 44392 | 74147142 | 74148866 | 198780 |
| 44393 | 74161187 | 74162948 | 271714, 198780 |
| 44394 | 74191498 | 74192130 | |
| 44395 | 74212136 | 74213234 | |
| 44396 | 74213370 | 74214645 | 249856 |
| 44397 | 74230480 | 74231777 | 249856 |
| 44398 | 74242078 | 74243133 | 249856 |
| 44399 | 74244306 | 74246074 | 249856 |
| 44400 | 74249526 | 74251007 | 249856 |
| 44401 | 74253187 | 74254549 | 249856 |
| 44402 | 74339838 | 74340833 | |
| 44403 | 74346934 | 74347800 | 250889 |
| 44404 | 74348048 | 74350784 | 250889 |
| 44405 | 74391217 | 74391523 | 145700 |
| 44406 | 74531727 | 74533293 | 145700 |
| 44407 | 74620867 | 74621518 | 247372 |
| 44408 | 74631421 | 74634121 | 113161, 247372 |
| 44409 | 74659444 | 74660005 | 271815 |
| 44410 | 74806116 | 74808302 | 122008, 113163 |
| 44411 | 74906959 | 74908008 | 189045 |
| 44412 | 74916276 | 74917100 | 189045 |
| 44413 | 74918137 | 74919025 | 189045 |
| 44414 | 74919415 | 74920330 | 189045 |
| 44415 | 74964629 | 74965693 | 189045 |
| 44416 | 74976500 | 74977543 | 152359 |
| 44417 | 75012248 | 75013510 | 152359 |
| 44418 | 75016651 | 75017628 | |
| 44419 | 75565780 | 75566429 | 122012 |
| 44420 | 75569188 | 75569874 | 122012 |
| 44421 | 75599076 | 75600841 | 250348, 122012 |
| 44422 | 75627411 | 75628864 | 266045, 122012 |
| 44423 | 75667659 | 75669251 | |
| 44424 | 75681909 | 75683068 | |
| 44425 | 75685513 | 75686084 | |
| 44426 | 75697853 | 75701185 | 145703 |
| 44427 | 75717339 | 75718572 | 145703 |
| 44428 | 75748344 | 75748914 | 145703 |
| 44429 | 75753553 | 75754757 | 145703 |
| 44430 | 75766821 | 75769352 | 145703 |
| 44431 | 75788674 | 75790572 | 145703 |
| 44432 | 75799788 | 75800257 | 145703 |
| 44433 | 75823441 | 75824647 | 145703 |
| 44434 | 75832513 | 75833738 | 145703 |
| 44435 | 75997020 | 75997540 | 225407, 145703 |
| 44436 | 76011606 | 76012355 | 181104, 225407 |
| 44437 | 76080962 | 76082132 | |
| 44438 | 76082907 | 76083609 | |
| 44439 | 76085459 | 76087689 | |
| 44440 | 76114416 | 76116717 | 164251 |
| 44441 | 76126737 | 76127375 | 164251 |
| 44442 | 76318497 | 76319320 | |
| 44443 | 76325780 | 76327295 | 164252 |
| 44444 | 76382044 | 76383997 | 250802, 132846 |
| 44445 | 76399211 | 76399757 | 250802 |
| 44446 | 76686325 | 76687370 | 113231 |
| 44447 | 76721696 | 76723481 | 164253, 113231 |
| 44448 | 76786992 | 76788675 | 164253 |
| 44449 | 77053886 | 77054777 | 171530 |
| 44450 | 77065992 | 77066915 | 171530 |
| 44451 | 77070702 | 77074329 | 171530 |
| 44452 | 77147476 | 77148266 | 171530 |
| 44453 | 77175695 | 77176251 | |
| 44454 | 77305882 | 77306642 | 132842 |
| 44455 | 77589743 | 77590986 | 132842 |
| 44456 | 77655348 | 77657487 | 085365, 245556 |
| 44457 | 77716330 | 77716824 | 085365 |
| 44458 | 77800520 | 77801122 | 145685 |
| 44459 | 77805811 | 77806609 | 145685 |
| 44460 | 77814008 | 77815759 | 145685 |
| 44461 | 77825506 | 77826767 | 145685 |
| 44462 | 77886883 | 77888164 | 145685 |
| 44463 | 77890073 | 77891445 | 145685 |
| 44464 | 77943832 | 77945325 | 145685 |
| 44465 | 78023168 | 78024576 | 145685 |
| 44466 | 78091563 | 78092110 | 113273 |
| 44467 | 78277076 | 78277878 | 113273 |
| 44468 | 78279965 | 78282650 | 113273 |
| 44469 | 78326429 | 78328863 | 132837 |
| 44470 | 78346237 | 78346846 | 132837 |
| 44471 | 78364395 | 78367613 | 132840, 132837 |
| 44472 | 78369762 | 78374182 | 132840, 132837 |
| 44473 | 78376423 | 78378971 | 132840, 132837 |
| 44474 | 78389432 | 78390208 | 132837 |
| 44475 | 78407074 | 78407875 | 145692, 132837 |
| 44476 | 78439499 | 78440269 | 132837 |
| 44477 | 78489443 | 78489933 | 132837 |
| 44478 | 78530565 | 78533664 | 152409, 132837 |
| 44479 | 78614781 | 78615254 | 152409 |
| 44480 | 78808983 | 78811089 | 152413 |
| 44481 | 78907412 | 78909421 | 164329 |
| 44482 | 78949047 | 78950398 | 164329 |
| 44483 | 78985309 | 78986310 | 164309 |
| 44484 | 79049512 | 79052519 | 164309 |
| 44485 | 79286058 | 79287630 | 113296, 177034 |
| 44486 | 79477144 | 79478112 | 164300 |
| 44487 | 79478493 | 79481362 | 164300 |
| 44488 | 79488357 | 79489366 | 164300 |
| 44489 | 79500483 | 79501378 | 164300 |
| 44490 | 79503543 | 79504467 | 164300 |
| 44491 | 79510730 | 79512716 | 164300 |
| 44492 | 79524738 | 79528107 | 164300 |
| 44493 | 79542219 | 79544512 | 164300 |
| 44494 | 79547258 | 79547877 | 164300 |
| 44495 | 79548626 | 79553482 | 164300 |
| 44496 | 79702636 | 79704805 | 039319 |
| 44497 | 79783526 | 79784753 | 152380, 249042 |
| 44498 | 79919418 | 79920259 | |
| 44499 | 79949611 | 79951807 | 113318, 228716 |
| 44500 | 79963871 | 79965094 | 113318 |
| 44501 | 80042954 | 80043339 | 113318 |
| 44502 | 80180750 | 80182083 | |
| 44503 | 80262716 | 80264761 | 113319 |
| 44504 | 80564481 | 80565613 | 247572 |
| 44505 | 80596407 | 80598274 | 131732, 247572 |
| 44506 | 80605759 | 80606911 | 131732 |
| 44507 | 80611092 | 80611802 | |
| 44508 | 80646363 | 80646993 | 172497 |
| 44509 | 80648584 | 80650801 | 172497 |
| 44510 | 80658836 | 80659930 | 172497 |
| 44511 | 80688951 | 80691373 | 172497 |
| 44512 | 80717517 | 80719911 | 145687 |
| 44513 | 80922224 | 80922823 | 145687 |
| 44514 | 81031863 | 81032350 | 145687 |
| 44515 | 81046288 | 81048370 | 145687 |
| 44516 | 81051082 | 81051711 | |
| 44517 | 81069195 | 81070220 | |
| 44518 | 81074265 | 81076066 | |
| 44519 | 81147052 | 81148553 | 249483 |
| 44520 | 81266726 | 81269040 | 152348 |
| 44521 | 81368782 | 81369545 | 248192, 152348 |
| 44522 | 81401243 | 81401741 | 152348 |
| 44523 | 81443104 | 81444401 | 152348 |
| 44524 | 81458631 | 81462432 | 152348 |
| 44525 | 81572924 | 81574811 | 186468 |
| 44526 | 81661390 | 81662181 | 205464 |
| 44527 | 81682716 | 81683539 | 205464 |
| 44528 | 81844682 | 81845838 | |
| 44529 | 81880235 | 81880920 | |
| 44530 | 81918254 | 81919482 | |
| 44531 | 81930553 | 81932113 | |
| 44532 | 82372296 | 82374049 | 152422, 174695 |
| 44533 | 82859179 | 82860393 | 249835, 038427 |
| 44534 | 82872363 | 82873708 | 249835, 038427 |
| 44535 | 83122860 | 83123379 | |
| 44536 | 84588462 | 84589823 | |
| 44537 | 84691070 | 84691861 | |
| 44538 | 85012015 | 85013293 | |
| 44539 | 85756240 | 85757660 | |
| 44540 | 85913071 | 85914820 | 127184 |
| 44541 | 86563174 | 86564771 | 145715 |
| 44542 | 86688257 | 86690193 | 134480 |
| 44543 | 86707427 | 86708949 | 134480 |
| 44544 | 87124298 | 87125360 | |
| 44545 | 87195135 | 87195957 | |
| 44546 | 87436706 | 87437671 | |
| 44547 | 87563788 | 87565763 | 247828, 164180 |
| 44548 | 87733484 | 87734647 | 271904, 250156 |
| 44549 | 87763983 | 87764982 | 271904 |
| 44550 | 87765940 | 87767151 | 271904 |
| 44551 | 87770079 | 87770893 | 271904 |
| 44552 | 87797921 | 87799099 | |
| 44553 | 87812197 | 87812739 | 245526 |
| 44554 | 88610671 | 88611863 | 248309 |
| 44555 | 89633333 | 89634051 | |
| 44556 | 89704538 | 89706819 | 255647, 153140 |
| 44557 | 89768822 | 89771060 | 176055, 113356 |
| 44558 | 89824379 | 89826339 | 164199, 176018 |
| 44559 | 90161724 | 90164528 | 164199 |
| 44560 | 90172138 | 90172700 | 164199 |
| 44561 | 90184276 | 90185389 | 164199 |
| 44562 | 90193321 | 90195872 | 164199 |
| 44563 | 90210577 | 90211946 | 164199 |
| 44564 | 90225736 | 90228425 | 164199 |
| 44565 | 90234920 | 90236208 | 164199 |
| 44566 | 90302457 | 90304559 | 164199 |
| 44567 | 90313788 | 90315142 | 164199 |
| 44568 | 90323255 | 90325000 | 164199 |
| 44569 | 90325542 | 90328349 | 249514, 164199 |
| 44570 | 90344895 | 90345993 | 164199 |
| 44571 | 90353709 | 90355676 | 164199 |
| 44572 | 90397337 | 90398144 | 164199 |
| 44573 | 90428271 | 90429606 | 164199 |
| 44574 | 90431971 | 90432610 | 164199 |
| 44575 | 90442094 | 90442478 | 164199 |
| 44576 | 90458412 | 90459188 | 164199 |
| 44577 | 90531020 | 90531769 | |
| 44578 | 90574976 | 90577695 | 234292 |
| 44579 | 90606700 | 90610598 | 271762, 248323 |
| 44580 | 90674576 | 90681204 | 113369 |
| 44581 | 90731950 | 90732571 | |
| 44582 | 91688495 | 91690536 | 250049 |
| 44583 | 91705303 | 91708887 | 250049 |
| 44584 | 91887992 | 91888995 | 249776 |
| 44585 | 91976630 | 91977736 | 249776 |
| 44586 | 92867905 | 92868901 | 237187 |
| 44587 | 92916797 | 92917464 | 237187 |
| 44588 | 92918368 | 92919259 | 175745, 237187 |
| 44589 | 92973148 | 92974068 | 113391 |
| 44590 | 93446417 | 93448075 | 113391 |
| 44591 | 93552903 | 93554662 | 185261 |
| 44592 | 93574308 | 93578592 | 185261 |
| 44593 | 93767989 | 93770024 | 185261 |
| 44594 | 93953586 | 93954887 | 133302, 270133, 185261 |
| 44595 | 94304045 | 94304642 | 175471 |
| 44596 | 94889816 | 94891586 | 164291, 198677 |
| 44597 | 94931189 | 94931643 | 164291 |
| 44598 | 94955972 | 94957623 | 178015 |
| 44599 | 94976462 | 94978581 | |
| 44600 | 94982139 | 94983425 | 175449 |
| 44601 | 95004608 | 95006188 | 145757, 175449 |
| 44602 | 95006212 | 95007191 | 145757, 175449 |
| 44603 | 95066355 | 95068379 | 250240, 164292 |
| 44604 | 95106781 | 95107435 | 173221, 164292 |
| 44605 | 95120136 | 95120896 | 173221, 164292 |
| 44606 | 95138891 | 95140406 | 173221, 164292 |
| 44607 | 95145060 | 95145750 | 173221, 164292 |
| 44608 | 95145879 | 95147010 | 173221, 164292 |
| 44609 | 95157037 | 95160988 | 173221, 164292 |
| 44610 | 95162547 | 95163882 | |
| 44611 | 95170002 | 95172773 | 251000, 251409 |
| 44612 | 95176644 | 95179938 | 251409 |
| 44613 | 95185522 | 95186782 | 272021, 251409 |
| 44614 | 95191896 | 95199865 | 250362, 236882 |
| 44615 | 95208161 | 95209931 | 250362 |
| 44616 | 95215622 | 95216542 | |
| 44617 | 95260348 | 95260898 | 118985 |
| 44618 | 95293485 | 95300384 | 251314, 118985 |
| 44619 | 95304601 | 95306323 | 250955, 251314 |
| 44620 | 95322230 | 95324306 | 251314 |
| 44621 | 95336789 | 95337488 | 251314 |
| 44622 | 95359173 | 95359967 | 251314 |
| 44623 | 95372188 | 95373773 | 251314 |
| 44624 | 95413472 | 95417818 | 207578, 250551, 251314 |
| 44625 | 95419805 | 95421844 | 250551, 251314 |
| 44626 | 95427696 | 95431240 | 250551, 251314 |
| 44627 | 95446242 | 95447550 | 250551, 251314 |
| 44628 | 95449140 | 95451549 | 250551, 251314 |
| 44629 | 95453101 | 95455079 | 250551, 251314 |
| 44630 | 95458926 | 95462382 | 250551, 251314 |
| 44631 | 95534516 | 95536448 | 250551, 251314 |
| 44632 | 95549830 | 95551109 | 249746, 250551, 251314 |
| 44633 | 95562480 | 95564010 | 251314 |
| 44634 | 95580849 | 95581877 | 251314 |
| 44635 | 95590605 | 95591205 | 250158, 251314 |
| 44636 | 95593394 | 95596258 | 250158, 251314 |
| 44637 | 95601548 | 95602392 | 250158, 251314 |
| 44638 | 95627519 | 95629478 | 251314 |
| 44639 | 95632769 | 95634762 | 251314 |
| 44640 | 95651931 | 95652847 | 251314 |
| 44641 | 95667323 | 95669709 | 251314 |
| 44642 | 95670956 | 95677118 | 251314 |
| 44643 | 95683463 | 95684080 | 251314 |
| 44644 | 95996992 | 95999311 | 153113 |
| 44645 | 96000361 | 96000801 | 153113 |
| 44646 | 96004316 | 96004954 | 153113 |
| 44647 | 96006054 | 96007005 | 153113 |
| 44648 | 96026864 | 96028893 | 153113 |
| 44649 | 96035919 | 96040200 | 153113 |
| 44650 | 96050321 | 96051320 | 153113 |
| 44651 | 96123747 | 96124618 | 164307 |
| 44652 | 96137403 | 96137859 | 164307 |
| 44653 | 96142441 | 96144513 | 164307 |
| 44654 | 96190814 | 96191617 | 247121 |
| 44655 | 96211440 | 96213024 | 164308, 247121 |
| 44656 | 96269601 | 96273000 | 113441, 247121 |
| 44657 | 96298820 | 96299536 | 113441 |
| 44658 | 96518010 | 96520218 | 251513, 058729, 251606 |
| 44659 | 98104589 | 98106749 | 246763, 174136 |
| 44660 | 98108642 | 98110617 | 246763, 174136 |
| 44661 | 98115089 | 98115776 | 174136 |
| 44662 | 98208296 | 98208910 | 153922 |
| 44663 | 98263536 | 98266118 | 248489 |
| 44664 | 98360782 | 98361927 | |
| 44665 | 98367899 | 98369973 | |
| 44666 | 101631898 | 101632460 | 173930 |
| 44667 | 102090166 | 102091974 | 145730 |
| 44668 | 102124405 | 102125119 | 145730 |
| 44669 | 102131534 | 102132591 | 145730 |
| 44670 | 102454144 | 102456935 | 145725, 145723 |
| 44671 | 102473991 | 102474651 | 145725 |
| 44672 | 102594024 | 102595424 | 181751 |
| 44673 | 102858983 | 102859395 | 226926 |
| 44674 | 102897237 | 102899251 | 112874 |
| 44675 | 102904410 | 102906304 | |
| 44676 | 102937796 | 102938647 | |
| 44677 | 102959715 | 102960755 | |
| 44678 | 103521396 | 103522288 | |
| 44679 | 106877734 | 106879223 | 184349 |
| 44680 | 107338450 | 107339372 | 145743 |
| 44681 | 107715895 | 107719028 | 145743 |
| 44682 | 107991640 | 107992525 | |
| 44683 | 107996699 | 107998001 | |
| 44684 | 108063257 | 108064722 | 272523 |
| 44685 | 108082981 | 108085325 | 151422 |
| 44686 | 108654054 | 108654905 | 249476 |
| 44687 | 108743782 | 108747535 | 198961 |
| 44688 | 109024165 | 109027613 | 112893 |
| 44689 | 109044506 | 109045512 | 112893 |
| 44690 | 109078152 | 109081001 | 112893 |
| 44691 | 109140971 | 109142097 | 112893 |
| 44692 | 109236501 | 109238018 | |
| 44693 | 109323361 | 109324082 | |
| 44694 | 109324616 | 109325328 | |
| 44695 | 109325898 | 109327080 | |
| 44696 | 109335711 | 109337384 | |
| 44697 | 109419561 | 109420110 | |
| 44698 | 109422122 | 109423888 | |
| 44699 | 109440988 | 109441825 | |
| 44700 | 109442126 | 109443321 | |
| 44701 | 109445396 | 109445872 | |
| 44702 | 109682297 | 109683300 | 186952 |
| 44703 | 110074102 | 110075709 | 186952, 164209 |
| 44704 | 110245521 | 110246296 | |
| 44705 | 110251336 | 110253399 | |
| 44706 | 110303022 | 110305692 | |
| 44707 | 110339112 | 110339813 | 248428 |
| 44708 | 110408284 | 110411567 | 145777 |
| 44709 | 110427629 | 110429481 | 253613, 134987 |
| 44710 | 110847108 | 110849500 | 246859, 164211 |
| 44711 | 110970841 | 110972016 | 246859 |
| 44712 | 110990550 | 110991544 | 246859 |
| 44713 | 111016281 | 111017349 | 134986, 246859 |
| 44714 | 111073311 | 111074483 | 134986, 246859 |
| 44715 | 111077979 | 111079066 | 134986 |
| 44716 | 111082556 | 111085179 | 134986 |
| 44717 | 111092379 | 111094231 | 134986 |
| 44718 | 111145514 | 111146180 | 134986 |
| 44719 | 111495952 | 111497684 | 224032, 238363, 129595 |
| 44720 | 111564079 | 111565148 | 129595, 251076 |
| 44721 | 111674135 | 111675018 | 129595 |
| 44722 | 111753439 | 111754350 | 129595 |
| 44723 | 111868881 | 111869793 | |
| 44724 | 112042786 | 112044284 | 134982 |
| 44725 | 112097798 | 112098297 | 134982 |
| 44726 | 112196172 | 112198198 | 272869, 153037, 258864 |
| 44727 | 112248979 | 112249588 | 129625 |
| 44728 | 112256027 | 112258356 | 129625 |
| 44729 | 112293955 | 112294640 | |
| 44730 | 112311824 | 112313478 | 172795 |
| 44731 | 112417334 | 112418523 | 171444 |
| 44732 | 112476745 | 112477481 | 171444 |
| 44733 | 112497833 | 112500337 | 171444 |
| 44734 | 112512100 | 112513388 | 171444 |
| 44735 | 112529542 | 112530456 | 171444 |
| 44736 | 112535131 | 112535786 | 171444 |
| 44737 | 112537416 | 112540985 | 171444 |
| 44738 | 112541058 | 112542024 | 171444 |
| 44739 | 112553408 | 112554522 | 171444 |
| 44740 | 112599742 | 112600386 | 171444 |
| 44741 | 112628805 | 112631172 | 171444 |
| 44742 | 112648526 | 112649286 | 171444 |
| 44743 | 112655358 | 112657369 | 171444 |
| 44744 | 112665595 | 112667201 | 171444, 232633 |
| 44745 | 112824077 | 112824785 | 171444 |
| 44746 | 112824915 | 112825437 | |
| 44747 | 112848820 | 112850592 | 047188 |
| 44748 | 112855876 | 112856764 | 047188 |
| 44749 | 113696520 | 113699170 | 080709 |
| 44750 | 113747038 | 113747527 | 080709 |
| 44751 | 114386310 | 114387062 | |
| 44752 | 114404033 | 114405478 | |
| 44753 | 114504638 | 114506264 | 152503 |
| 44754 | 114530095 | 114531224 | |
| 44755 | 114597582 | 114599548 | 271797, 164219 |
| 44756 | 114631520 | 114632555 | 164221 |
| 44757 | 114659689 | 114660248 | |
| 44758 | 114673364 | 114674295 | |
| 44759 | 114678700 | 114680870 | |
| 44760 | 114683358 | 114686761 | |
| 44761 | 114711069 | 114712462 | |
| 44762 | 114761903 | 114762673 | |
| 44763 | 114786083 | 114786930 | |
| 44764 | 114794973 | 114798413 | |
| 44765 | 114877791 | 114881043 | 145780 |
| 44766 | 114891509 | 114892556 | |
| 44767 | 114892603 | 114896947 | |
| 44768 | 114937203 | 114938449 | 249249, 243414, 251201 |
| 44769 | 114960846 | 114962662 | 243414, 251201, 134970 |
| 44770 | 114969863 | 114971116 | |
| 44771 | 115044172 | 115046406 | |
| 44772 | 115074227 | 115075233 | |
| 44773 | 115084016 | 115085535 | |
| 44774 | 115132227 | 115132727 | |
| 44775 | 115133661 | 115134574 | |
| 44776 | 115150972 | 115153287 | 129596 |
| 44777 | 115176308 | 115178716 | 177879, 145782 |
| 44778 | 115419900 | 115421791 | 145781, 271918 |
| 44779 | 115608170 | 115608877 | 145781 |
| 44780 | 115609147 | 115609578 | 145781 |
| 44781 | 115706433 | 115707859 | 145781 |
| 44782 | 115860532 | 115861029 | 092421 |
| 44783 | 118323106 | 118324723 | 169570 |
| 44784 | 118344570 | 118345929 | 250678, 249494 |
| 44785 | 118371332 | 118372548 | 249494 |
| 44786 | 118372721 | 118373873 | 172869, 249494 |
| 44787 | 118390481 | 118391544 | 172869, 249494 |
| 44788 | 118405375 | 118407999 | 249494, 172869 |
| 44789 | 118554460 | 118555603 | 172869 |
| 44790 | 118603581 | 118605425 | 145779 |
| 44791 | 118609771 | 118611439 | 145779 |
| 44792 | 118637617 | 118640394 | 145779 |
| 44793 | 118658722 | 118661144 | 145779 |
| 44794 | 118670490 | 118671122 | 145779 |
| 44795 | 118691352 | 118691976 | 145779 |
| 44796 | 118732720 | 118736098 | 145779 |
| 44797 | 118787711 | 118789359 | 133835 |
| 44798 | 119474656 | 119475374 | |
| 44799 | 121296906 | 121298756 | 151304 |
| 44800 | 121498493 | 121501915 | 164185, 250803 |
| 44801 | 121814208 | 121816259 | 250328 |
| 44802 | 122110322 | 122111710 | 205302 |
| 44803 | 122180174 | 122182339 | 064652 |
| 44804 | 122371251 | 122373370 | 249996, 168938 |
| 44805 | 122718005 | 122718376 | 168944 |
| 44806 | 122758013 | 122760392 | 168944 |
| 44807 | 122764292 | 122766096 | |
| 44808 | 122791863 | 122792394 | |
| 44809 | 122795854 | 122796456 | |
| 44810 | 122841254 | 122841762 | |
| 44811 | 122846942 | 122849300 | 151292 |
| 44812 | 123862746 | 123863829 | 248296 |
| 44813 | 123916719 | 123917381 | |
| 44814 | 123932333 | 123933227 | |
| 44815 | 123934557 | 123935853 | |
| 44816 | 123991399 | 123992237 | 168916 |
| 44817 | 123996122 | 123998729 | 168916 |
| 44818 | 124019418 | 124020199 | 168916 |
| 44819 | 124071908 | 124073086 | 168916 |
| 44820 | 124078498 | 124080737 | 168916 |
| 44821 | 124081311 | 124085495 | 168916 |
| 44822 | 124119313 | 124119813 | |
| 44823 | 124223215 | 124224300 | |
| 44824 | 124242241 | 124243075 | |
| 44825 | 124268248 | 124269197 | |
| 44826 | 124346800 | 124347751 | |
| 44827 | 124376481 | 124377402 | 250411 |
| 44828 | 124379189 | 124380874 | |
| 44829 | 124385736 | 124386455 | |
| 44830 | 124613113 | 124613786 | |
| 44831 | 124652759 | 124653784 | |
| 44832 | 124744242 | 124745515 | |
| 44833 | 124746181 | 124747398 | |
| 44834 | 125337737 | 125338641 | |
| 44835 | 125693242 | 125694171 | |
| 44836 | 125698859 | 125702139 | 155324 |
| 44837 | 125705122 | 125708091 | 155324 |
| 44838 | 125758491 | 125759704 | 155324 |
| 44839 | 125805432 | 125806213 | 155324, 250602 |
| 44840 | 125807097 | 125808613 | 155324, 250602 |
| 44841 | 125878926 | 125880297 | 164904 |
| 44842 | 125930450 | 125931465 | 164904 |
| 44843 | 125935976 | 125937652 | 164902 |
| 44844 | 125959579 | 125960799 | 164902 |
| 44845 | 125984714 | 125986620 | |
| 44846 | 126111815 | 126113949 | 113368, 251072 |
| 44847 | 126365651 | 126367085 | 173926 |
| 44848 | 126408328 | 126409788 | 164241 |
| 44849 | 126852590 | 126854394 | 164244 |
| 44850 | 127270314 | 127271154 | 230561 |
| 44851 | 127314404 | 127315135 | 245937 |
| 44852 | 127417914 | 127420590 | 064651, 245937 |
| 44853 | 127534623 | 127535270 | |
| 44854 | 128429817 | 128431558 | 066583 |
| 44855 | 128434215 | 128434784 | 066583 |
| 44856 | 128437357 | 128438125 | 066583 |
| 44857 | 128461370 | 128462735 | |
| 44858 | 129103475 | 129104048 | 251680 |
| 44859 | 129239454 | 129241551 | 198108, 251680 |
| 44860 | 129241681 | 129242694 | 198108 |
| 44861 | 130498740 | 130501355 | 169567 |
| 44862 | 130505691 | 130507346 | 186687, 169567 |
| 44863 | 130598680 | 130601552 | 158985 |
| 44864 | 130616995 | 130618296 | 158985 |
| 44865 | 130639168 | 130639583 | 158985 |
| 44866 | 130714749 | 130715831 | 158985 |
| 44867 | 130729056 | 130730812 | 158985 |
| 44868 | 130742717 | 130743423 | |
| 44869 | 130834098 | 130835135 | 158987, 273217 |
| 44870 | 130969979 | 130972539 | 158987, 273217 |
| 44871 | 131007425 | 131008418 | 273217, 217128 |
| 44872 | 131020075 | 131020858 | 273217, 217128 |
| 44873 | 131131381 | 131133917 | 273217, 217128 |
| 44874 | 131332304 | 131332918 | 164398 |
| 44875 | 131429471 | 131430888 | |
| 44876 | 131438295 | 131439929 | |
| 44877 | 131562583 | 131564344 | 072682 |
| 44878 | 131628441 | 131630311 | 197208, 072682 |
| 44879 | 131698320 | 131701513 | 263597, 233006 |
| 44880 | 131703683 | 131706853 | 197375, 233006 |
| 44881 | 131722144 | 131724569 | 197375 |
| 44882 | 131745483 | 131748071 | 197536 |
| 44883 | 131750098 | 131751366 | 197536 |
| 44884 | 131752527 | 131753146 | 197536 |
| 44885 | 131760487 | 131763701 | 238160, 197536 |
| 44886 | 131770241 | 131771567 | 197536 |
| 44887 | 131791697 | 131797263 | 197536 |
| 44888 | 131798951 | 131800775 | 197536 |
| 44889 | 131802294 | 131803558 | 197536 |
| 44890 | 131810225 | 131812975 | 197536 |
| 44891 | 131816423 | 131817626 | 125347 |
| 44892 | 131824278 | 131827582 | 125347 |
| 44893 | 131830749 | 131838806 | |
| 44894 | 131891886 | 131894047 | 113525, 113522 |
| 44895 | 131897293 | 131897952 | 113522 |
| 44896 | 132072615 | 132073580 | 131437 |
| 44897 | 132112102 | 132113694 | 164402 |
| 44898 | 132142681 | 132143559 | 164402 |
| 44899 | 132155577 | 132156810 | |
| 44900 | 132164790 | 132167390 | 164403 |
| 44901 | 132172746 | 132177622 | |
| 44902 | 132188096 | 132191000 | |
| 44903 | 132201157 | 132203509 | 164405, 164404 |
| 44904 | 132206400 | 132212503 | 164406, 072364 |
| 44905 | 132241610 | 132242451 | 072364 |
| 44906 | 132297467 | 132300418 | 272023, 072364 |
| 44907 | 132342011 | 132342998 | 155329 |
| 44908 | 132351833 | 132353195 | 155329 |
| 44909 | 132361203 | 132362577 | 155329 |
| 44910 | 132384568 | 132385934 | |
| 44911 | 132385988 | 132388936 | 170606 |
| 44912 | 132556739 | 132557376 | 248245, 053108 |
| 44913 | 132560573 | 132562535 | 053108 |
| 44914 | 132567671 | 132568588 | 053108 |
| 44915 | 132620699 | 132621806 | 053108 |
| 44916 | 132759296 | 132760020 | 053108 |
| 44917 | 132792508 | 132793329 | 053108 |
| 44918 | 132818876 | 132819909 | 053108 |
| 44919 | 132841873 | 132843074 | 053108 |
| 44920 | 132845186 | 132845728 | 053108 |
| 44921 | 132946553 | 132947235 | 053108 |
| 44922 | 133078354 | 133078811 | |
| 44923 | 133152874 | 133155110 | |
| 44924 | 133156669 | 133157569 | |
| 44925 | 133160203 | 133160978 | |
| 44926 | 133288111 | 133289819 | |
| 44927 | 133303351 | 133306457 | 113583 |
| 44928 | 133338445 | 133342312 | 271737, 213585 |
| 44929 | 133353640 | 133356355 | |
| 44930 | 133428480 | 133429104 | |
| 44931 | 133430316 | 133430912 | |
| 44932 | 133434123 | 133435443 | |
| 44933 | 133450097 | 133450882 | 081059 |
| 44934 | 133488959 | 133489819 | 113558 |
| 44935 | 133511196 | 133513688 | 113558, 272772 |
| 44936 | 133560538 | 133563174 | 266751, 270177, 113575, |
| | | | 272772, 006837, 273345 |
| 44937 | 133582898 | 133584151 | 006837, 273345 |
| 44938 | 133588345 | 133588999 | 006837, 273345 |
| 44939 | 133635077 | 133635951 | 006837, 273345 |
| 44940 | 133702156 | 133703709 | 006837, 273345 |
| 44941 | 133705897 | 133708582 | 119048, 006837, 273345 |
| 44942 | 133746530 | 133748743 | 237190 |
| 44943 | 133751570 | 133752739 | |
| 44944 | 133770566 | 133775490 | 250994, 250564 |
| 44945 | 133800634 | 133803242 | 250994 |
| 44946 | 133838351 | 133840459 | |
| 44947 | 133841405 | 133843560 | 251169 |
| 44948 | 133844184 | 133848884 | 251169 |
| 44949 | 133859415 | 133863962 | 043143 |
| 44950 | 133889881 | 133891173 | 043143 |
| 44951 | 133926484 | 133929034 | |
| 44952 | 133944253 | 133945609 | 152700 |
| 44953 | 133961951 | 133962624 | 152700 |
| 44954 | 133967625 | 133969194 | 152700 |
| 44955 | 133974164 | 133975006 | 152700 |
| 44956 | 133983094 | 133986189 | 113615, 152700 |
| 44957 | 134073199 | 134075394 | 164615 |
| 44958 | 134093927 | 134095257 | 145833 |
| 44959 | 134142424 | 134143173 | 145833 |
| 44960 | 134172708 | 134174763 | 145833 |
| 44961 | 134177896 | 134179148 | 145833 |
| 44962 | 134180709 | 134183624 | 181904, 145833 |
| 44963 | 134209293 | 134211130 | 113621 |
| 44964 | 134239732 | 134241820 | 132570 |
| 44965 | 134349916 | 134350449 | |
| 44966 | 134646016 | 134646958 | 224186 |
| 44967 | 134733704 | 134735822 | 270021, 113648 |
| 44968 | 135288189 | 135291053 | 145826 |
| 44969 | 135308390 | 135310764 | |
| 44970 | 135312401 | 135314311 | |
| 44971 | 135351697 | 135357772 | |
| 44972 | 135364100 | 135366326 | 120708 |
| 44973 | 135371500 | 135371927 | 120708 |
| 44974 | 135387634 | 135391191 | 120708 |
| 44975 | 135391661 | 135396343 | 120708 |
| 44976 | 135401967 | 135403577 | |
| 44977 | 135415858 | 135418929 | 270123 |
| 44978 | 135428360 | 135432842 | |
| 44979 | 135458354 | 135460324 | |
| 44980 | 135467537 | 135469737 | 113658, 164621 |
| 44981 | 135485505 | 135486052 | 113658 |
| 44982 | 135763189 | 135763578 | |
| 44983 | 136957249 | 136958935 | 146021 |
| 44984 | 137001937 | 137002635 | 146021 |
| 44985 | 137002804 | 137004710 | 146021 |
| 44986 | 137028858 | 137029578 | 146021 |
| 44987 | 137069427 | 137070221 | 146021 |
| 44988 | 137088178 | 137091278 | 177733 |
| 44989 | 137094276 | 137095080 | |
| 44990 | 137169019 | 137169565 | 250159 |
| 44991 | 137335073 | 137337214 | 031003 |
| 44992 | 137367888 | 137369948 | 246323, 031003 |
| 44993 | 137513605 | 137514987 | 112984, 112983 |
| 44994 | 137548245 | 137549462 | 094880 |
| 44995 | 137558421 | 137559010 | |
| 44996 | 137577321 | 137578122 | |
| 44997 | 137606795 | 137607525 | 146013 |
| 44998 | 137670471 | 137671017 | 158402, 120709 |
| 44999 | 137672501 | 137674958 | 158402, 120709 |
| 45000 | 137688059 | 137689691 | 120733 |
| 45001 | 137798992 | 137802572 | 120738 |
| 45002 | 137805775 | 137807024 | |
| 45003 | 137827373 | 137829640 | |
| 45004 | 137840380 | 137840956 | |
| 45005 | 137877435 | 137880613 | 120705 |
| 45006 | 137909630 | 137912318 | 113013 |
| 45007 | 137919332 | 137920578 | |
| 45008 | 138061374 | 138062181 | 044115 |
| 45009 | 138066592 | 138067252 | 044115 |
| 45010 | 138087943 | 138090400 | 253404, 044115 |
| 45011 | 138263399 | 138264157 | 044115 |
| 45012 | 138350141 | 138351218 | 249593, 120725 |
| 45013 | 138396381 | 138398821 | 120725 |
| 45014 | 138400088 | 138400757 | 120725 |
| 45015 | 138411113 | 138411789 | 120725 |
| 45016 | 138477165 | 138478202 | 120725 |
| 45017 | 138523594 | 138524663 | 120725 |
| 45018 | 138532951 | 138534514 | 120725 |
| 45019 | 138608986 | 138610681 | 015479, 120725 |
| 45020 | 138628947 | 138631149 | 120725, 015479 |
| 45021 | 138709089 | 138709946 | 170482 |
| 45022 | 138718109 | 138719383 | 170482 |
| 45023 | 138739275 | 138740476 | 170469 |
| 45024 | 138774354 | 138775494 | 170464 |
| 45025 | 138896923 | 138898309 | |
| 45026 | 138940061 | 138942571 | 131508 |
| 45027 | 139016563 | 139019044 | |
| 45028 | 139022248 | 139024323 | 272255 |
| 45029 | 139027131 | 139030523 | 250635, 171604 |
| 45030 | 139030588 | 139033900 | 171604 |
| 45031 | 139034530 | 139036310 | 171604 |
| 45032 | 139038464 | 139040686 | 171604 |
| 45033 | 139043749 | 139045628 | 171604 |
| 45034 | 139047330 | 139048872 | 171604 |
| 45035 | 139054401 | 139055606 | 171604 |
| 45036 | 139063967 | 139065374 | 249526 |
| 45037 | 139066175 | 139067764 | 249526 |
| 45038 | 139080358 | 139081199 | 249526 |
| 45039 | 139088496 | 139089178 | 249526 |
| 45040 | 139104329 | 139105192 | 249526 |
| 45041 | 139126567 | 139127971 | |
| 45042 | 139166944 | 139168329 | |
| 45043 | 139225319 | 139226405 | 158458 |
| 45044 | 139409287 | 139410063 | 158458 |
| 45045 | 139486391 | 139488032 | 185129, 245146 |
| 45046 | 139492624 | 139494708 | 185129 |
| 45047 | 139525224 | 139525778 | |
| 45048 | 139536273 | 139537320 | 254363 |
| 45049 | 139553965 | 139556377 | 120306 |
| 45050 | 139598167 | 139602982 | 120306 |
| 45051 | 139604856 | 139605444 | 120306 |
| 45052 | 139620374 | 139621384 | 120306 |
| 45053 | 139640771 | 139642401 | 113068, 120306 |
| 45054 | 139674339 | 139675596 | 113068 |
| 45055 | 139676996 | 139678807 | 113068 |
| 45056 | 139681704 | 139683684 | 113068 |
| 45057 | 139702684 | 139703370 | |
| 45058 | 139724208 | 139725134 | 113070 |
| 45059 | 139725336 | 139726701 | 113070 |
| 45060 | 139737010 | 139737718 | 253965 |
| 45061 | 139742707 | 139743327 | 113073 |
| 45062 | 139779854 | 139782471 | 254996, 131503, 249637 |
| 45063 | 139788428 | 139788972 | 254996, 131503 |
| 45064 | 139847617 | 139848187 | 254996, 131503 |
| 45065 | 139859354 | 139860381 | 254996, 131503 |
| 45066 | 139877316 | 139877995 | 254996, 131503 |
| 45067 | 139926003 | 139928559 | 243056, 254996, 213523, |
| | | | 131503 |
| 45068 | 139936054 | 139938176 | 113108, 213523 |
| 45069 | 139939691 | 139940350 | 113108 |
| 45070 | 139943089 | 139945782 | 176087, 113108 |
| 45071 | 139953524 | 139954367 | 113108 |
| 45072 | 139975087 | 139977482 | |
| 45073 | 139981509 | 139983724 | |
| 45074 | 139986414 | 139987072 | |
| 45075 | 140011038 | 140013256 | 170458 |
| 45076 | 140018430 | 140019909 | 113119, 131495 |
| 45077 | 140022383 | 140022820 | 131495, 113119 |
| 45078 | 140026462 | 140027920 | 264052, 113141, 131495 |
| 45079 | 140043678 | 140045197 | 120314 |
| 45080 | 140070003 | 140072132 | 112855, 170445 |
| 45081 | 140079635 | 140081085 | 146007 |
| 45082 | 140090045 | 140091278 | 199990 |
| 45083 | 140105305 | 140106133 | 202515 |
| 45084 | 140356049 | 140358641 | 204967, 204965, 204963, |
| | | | 204962, 204961, 204969, |
| | | | 081842, 204970, 239389, |
| | | | 255408, 243232, 249158, |
| | | | 251664, 248383, 250120 |
| 45085 | 140699667 | 140701067 | 178913, 255729 |
| 45086 | 140855250 | 140856280 | 240184, 253537, 081853, |
| | | | 254221, 262209, 253873, |
| | | | 204956, 261934, 253485, |
| | | | 253159, 253731, 253846, |
| | | | 254122, 254245, 253767, |
| | | | 262576, 253305, 253953, |
| | | | 253910 |
| 45087 | 140900585 | 140901768 | 131504 |
| 45088 | 140904146 | 140905901 | 131504 |
| 45089 | 140939586 | 140941027 | 246422, 131504 |
| 45090 | 140943632 | 140944743 | 246422, 131504 |
| 45091 | 140969383 | 140970516 | 131504 |
| 45092 | 140983778 | 140984234 | 131504 |
| 45093 | 140996733 | 140999262 | 228737, 131504 |
| 45094 | 141015370 | 141017569 | 164620, 171720 |
| 45095 | 141081598 | 141082833 | |
| 45096 | 141220838 | 141221651 | |
| 45097 | 141227748 | 141231005 | |
| 45098 | 141253372 | 141254928 | 156453 |
| 45099 | 141255042 | 141256929 | 156453 |
| 45100 | 141257186 | 141259223 | 156453 |
| 45101 | 141298599 | 141299684 | |
| 45102 | 141302435 | 141304026 | 081791 |
| 45103 | 141347819 | 141350234 | 254099, 113555, 013561 |
| 45104 | 141365661 | 141366035 | 013561 |
| 45105 | 141376981 | 141377730 | 113552 |
| 45106 | 141391584 | 141396033 | 113552 |
| 45107 | 141487623 | 141489669 | 131507 |
| 45108 | 141512266 | 141513652 | 131507 |
| 45109 | 141625462 | 141626435 | |
| 45110 | 141629382 | 141630246 | |
| 45111 | 141698642 | 141700242 | 187678 |
| 45112 | 141702099 | 141704989 | 231185, 187678 |
| 45113 | 141735710 | 141737708 | 231185 |
| 45114 | 141739977 | 141741023 | 231185 |
| 45115 | 141742176 | 141742861 | 231185 |
| 45116 | 141879109 | 141879897 | 231185 |
| 45117 | 141890787 | 141893023 | 231185 |
| 45118 | 141901055 | 141901920 | 231185 |
| 45119 | 141909096 | 141910406 | 231185 |
| 45120 | 142059558 | 142061126 | 113578 |
| 45121 | 142092247 | 142093379 | |
| 45122 | 142096456 | 142097697 | |
| 45123 | 142149191 | 142150972 | 145819 |
| 45124 | 142187037 | 142187515 | 145819 |
| 45125 | 142197325 | 142198155 | 145819 |
| 45126 | 142200825 | 142202165 | 145819 |
| 45127 | 142254002 | 142254674 | 145819 |
| 45128 | 142300185 | 142301057 | 145819 |
| 45129 | 142338970 | 142342319 | 145819 |
| 45130 | 142361248 | 142362352 | 145819 |
| 45131 | 142430896 | 142431716 | 145819 |
| 45132 | 142485649 | 142486165 | 145819 |
| 45133 | 142487887 | 142488823 | 145819 |
| 45134 | 142520142 | 142520929 | 145819 |
| 45135 | 142545820 | 142547008 | 145819 |
| 45136 | 142562057 | 142563533 | 145819 |
| 45137 | 142579405 | 142580900 | 145819 |
| 45138 | 142599133 | 142599741 | 145819 |
| 45139 | 142608149 | 142610482 | 145819 |
| 45140 | 142610515 | 142612110 | |
| 45141 | 142612692 | 142615752 | |
| 45142 | 142619061 | 142620738 | |
| 45143 | 142623389 | 142624472 | |
| 45144 | 142624799 | 142626529 | |
| 45145 | 142628054 | 142630495 | |
| 45146 | 142659125 | 142659682 | 113580 |
| 45147 | 142696544 | 142697195 | 113580 |
| 45148 | 142727118 | 142727846 | 113580 |
| 45149 | 142735280 | 142736012 | 113580 |
| 45150 | 142756506 | 142757088 | 113580 |
| 45151 | 142779844 | 142780791 | 113580 |
| 45152 | 142781245 | 142785933 | 113580 |
| 45153 | 142791374 | 142792640 | 113580 |
| 45154 | 142797476 | 142798615 | 113580 |
| 45155 | 142811605 | 142815476 | 113580 |
| 45156 | 142861388 | 142862274 | |
| 45157 | 142870965 | 142872284 | 249429 |
| 45158 | 142873247 | 142876374 | 249429 |
| 45159 | 142885736 | 142888168 | 249429 |
| 45160 | 142893167 | 142893780 | 249429 |
| 45161 | 142913721 | 142914429 | |
| 45162 | 142915427 | 142916916 | |
| 45163 | 142923147 | 142923891 | |
| 45164 | 142933091 | 142935284 | |
| 45165 | 142939954 | 142943080 | 251205 |
| 45166 | 142949470 | 142950540 | |
| 45167 | 142973658 | 142977094 | |
| 45168 | 142979884 | 142981402 | |
| 45169 | 143008787 | 143010349 | 249881 |
| 45170 | 143032718 | 143033308 | 249881 |
| 45171 | 143053403 | 143054078 | 249881 |
| 45172 | 143059266 | 143059846 | 266478, 249881 |
| 45173 | 143062343 | 143063606 | 249881 |
| 45174 | 143129320 | 143129932 | 249881 |
| 45175 | 143549359 | 143550591 | 183775, 145817 |
| 45176 | 143830588 | 143832186 | 183775 |
| 45177 | 145213835 | 145215926 | 186314 |
| 45178 | 145304570 | 145305784 | |
| 45179 | 145315473 | 145318697 | 156463 |
| 45180 | 145323718 | 145327441 | 156463 |
| 45181 | 145345425 | 145346064 | 156463 |
| 45182 | 145353932 | 145357548 | 156463 |
| 45183 | 145357774 | 145360124 | 156463 |
| 45184 | 145360904 | 145362704 | 156463 |
| 45185 | 145561384 | 145562807 | 133706 |
| 45186 | 145581753 | 145584247 | 091009 |
| 45187 | 145593095 | 145593413 | 091009 |
| 45188 | 145662994 | 145664132 | 091009 |
| 45189 | 145826250 | 145828267 | 113649 |
| 45190 | 146130406 | 146131264 | 156475 |
| 45191 | 146547960 | 146550775 | |
| 45192 | 146554919 | 146555839 | |
| 45193 | 146557069 | 146557826 | |
| 45194 | 146833103 | 146833969 | 113657 |
| 45195 | 146992788 | 146993381 | 176049 |
| 45196 | 147024456 | 147025562 | 176049 |
| 45197 | 147050763 | 147051653 | 176049 |
| 45198 | 147152712 | 147153669 | 176049 |
| 45199 | 147180992 | 147181411 | |
| 45200 | 147214642 | 147216928 | |
| 45201 | 147217519 | 147218826 | |
| 45202 | 147285713 | 147286644 | 178776 |
| 45203 | 147365622 | 147366218 | |
| 45204 | 147391208 | 147391628 | |
| 45205 | 147525365 | 147527902 | |
| 45206 | 147762358 | 147764508 | 145868, 247199 |
| 45207 | 148061028 | 148061804 | |
| 45208 | 148205158 | 148206856 | 169252 |
| 45209 | 148220957 | 148222050 | |
| 45210 | 148294195 | 148295466 | |
| 45211 | 148340858 | 148342035 | 169247 |
| 45212 | 148344663 | 148346768 | 169247 |
| 45213 | 148346815 | 148348363 | 169247 |
| 45214 | 148363247 | 148364910 | 169247 |
| 45215 | 148381454 | 148382270 | 169247 |
| 45216 | 148384503 | 148385266 | 169247 |
| 45217 | 148398331 | 148399610 | 169247 |
| 45218 | 148416403 | 148417123 | 169247 |
| 45219 | 148493226 | 148493816 | |
| 45220 | 148509699 | 148511073 | |
| 45221 | 148511699 | 148517208 | |
| 45222 | 148520229 | 148524730 | 173210 |
| 45223 | 148528250 | 148537024 | 173210 |
| 45224 | 148538646 | 148543378 | 173210 |
| 45225 | 148553527 | 148554943 | 248647, 173210 |
| 45226 | 148574567 | 148576113 | 248647, 173210 |
| 45227 | 148582676 | 148586823 | 248647, 173210 |
| 45228 | 148606697 | 148609974 | 248647, 173210, 253406 |
| 45229 | 148641694 | 148642165 | 248647, 253406 |
| 45230 | 148650681 | 148651913 | 157510, 248647, 253406 |
| 45231 | 148724175 | 148725866 | 164284 |
| 45232 | 148862311 | 148862818 | |
| 45233 | 148865282 | 148866670 | |
| 45234 | 148928604 | 148932015 | 183111, 113712 |
| 45235 | 148960765 | 148961302 | 183111 |
| 45236 | 149019937 | 149021292 | |
| 45237 | 149102493 | 149104985 | |
| 45238 | 149109274 | 149113290 | 155846, 199047 |
| 45239 | 149126760 | 149127917 | 155846 |
| 45240 | 149128139 | 149130031 | 155846 |
| 45241 | 149177743 | 149179235 | 155846 |
| 45242 | 149207717 | 149208494 | 155846 |
| 45243 | 149237032 | 149237425 | |
| 45244 | 149339330 | 149341209 | 155850 |
| 45245 | 149379331 | 149381715 | 113716, 164296 |
| 45246 | 149516130 | 149520670 | 113721 |
| 45247 | 149521143 | 149523868 | 113721 |
| 45248 | 149564312 | 149565453 | |
| 45249 | 149643462 | 149644228 | 070808 |
| 45250 | 149696122 | 149697472 | 183876 |
| 45251 | 149736631 | 149738482 | 070814 |
| 45252 | 149791904 | 149792849 | 019582 |
| 45253 | 149807358 | 149808634 | |
| 45254 | 149827479 | 149829941 | 164587 |
| 45255 | 149838017 | 149839651 | |
| 45256 | 149843734 | 149844609 | |
| 45257 | 149851894 | 149853124 | |
| 45258 | 149864396 | 149869485 | 070614, 254333 |
| 45259 | 149871120 | 149872644 | 070614 |
| 45260 | 149889796 | 149890763 | 070614 |
| 45261 | 149891156 | 149892528 | 070614 |
| 45262 | 149894201 | 149896146 | 070614 |
| 45263 | 149896757 | 149898308 | 070614 |
| 45264 | 149900140 | 149903771 | 070614 |
| 45265 | 149919343 | 149923544 | 239191, 070614 |
| 45266 | 149927617 | 149928145 | 070614 |
| 45267 | 149938761 | 149941061 | |
| 45268 | 149961536 | 149962721 | |
| 45269 | 149979440 | 149980379 | |
| 45270 | 149989257 | 149990082 | 253852, 171992 |
| 45271 | 149993645 | 149994303 | 253852, 171992 |
| 45272 | 149994717 | 149996274 | 253852, 171992 |
| 45273 | 150003958 | 150005168 | 171992 |
| 45274 | 150017001 | 150023214 | 171992 |
| 45275 | 150024231 | 150025043 | 171992 |
| 45276 | 150035533 | 150036809 | 171992 |
| 45277 | 150036902 | 150039370 | 171992 |
| 45278 | 150079727 | 150081381 | 086589 |
| 45279 | 150137183 | 150139382 | 132912 |
| 45280 | 150142115 | 150143552 | |
| 45281 | 150383962 | 150385083 | |
| 45282 | 150396155 | 150396686 | |
| 45283 | 150399632 | 150401455 | 211445 |
| 45284 | 150414321 | 150423040 | 145901 |
| 45285 | 150438830 | 150440743 | 145901 |
| 45286 | 150441531 | 150444281 | 145901 |
| 45287 | 150446959 | 150449066 | 145901 |
| 45288 | 150452051 | 150452651 | 145901 |
| 45289 | 150455096 | 150456775 | 145901 |
| 45290 | 150456993 | 150466193 | 145901 |
| 45291 | 150472166 | 150476157 | 145901 |
| 45292 | 150479658 | 150480224 | |
| 45293 | 150480302 | 150482110 | 197043 |
| 45294 | 150483602 | 150485104 | 197043 |
| 45295 | 150487613 | 150488410 | 197043 |
| 45296 | 150535513 | 150537592 | 197043 |
| 45297 | 150544010 | 150544804 | |
| 45298 | 150588124 | 150589095 | 198624 |
| 45299 | 150602946 | 150604125 | 198624, 196743 |
| 45300 | 150614147 | 150614825 | 196743 |
| 45301 | 150629694 | 150631169 | 196743 |
| 45302 | 150631664 | 150632947 | 196743 |
| 45303 | 150649595 | 150650923 | 196743 |
| 45304 | 150826592 | 150828088 | 123643 |
| 45305 | 150997422 | 150998093 | |
| 45306 | 151134563 | 151135529 | 177556, 253921 |
| 45307 | 151137084 | 151138581 | 177556, 253921 |
| 45308 | 151149569 | 151152904 | 145907, 177556 |
| 45309 | 152286546 | 152287905 | 249484 |
| 45310 | 152336757 | 152337198 | 249484 |
| 45311 | 152373532 | 152374024 | 249484 |
| 45312 | 152825445 | 152826281 | |
| 45313 | 152934913 | 152935858 | 155511 |
| 45314 | 153417622 | 153419483 | 037749, 055147 |
| 45315 | 153456984 | 153458218 | 037749 |
| 45316 | 153568978 | 153570983 | 164574, 037749 |
| 45317 | 153665198 | 153667245 | 164574 |
| 45318 | 153667590 | 153668362 | 164574 |
| 45319 | 153747210 | 153748695 | 164574, 245275 |
| 45320 | 153824205 | 153826784 | 164576, 245275 |
| 45321 | 154024082 | 154024934 | |
| 45322 | 154026317 | 154028066 | |
| 45323 | 154028181 | 154028676 | |
| 45324 | 154032788 | 154034385 | |
| 45325 | 154061726 | 154063611 | |
| 45326 | 154080651 | 154081176 | |
| 45327 | 154081303 | 154081815 | |
| 45328 | 154088424 | 154089013 | |
| 45329 | 154092685 | 154093783 | 155506 |
| 45330 | 154093858 | 154094674 | 155506 |
| 45331 | 154119992 | 154120753 | 155506 |
| 45332 | 154132962 | 154138191 | 155506 |
| 45333 | 154158389 | 154159222 | 155506 |
| 45334 | 154195346 | 154196061 | 155506 |
| 45335 | 154213544 | 154215207 | 170271 |
| 45336 | 154216888 | 154217607 | 170271 |
| 45337 | 154229916 | 154230549 | 170271 |
| 45338 | 154236558 | 154239274 | 155508, 170271 |
| 45339 | 154316877 | 154318150 | 082516 |
| 45340 | 154319958 | 154321747 | 082515 |
| 45341 | 154798487 | 154799304 | |
| 45342 | 154835301 | 154836171 | |
| 45343 | 154839351 | 154840660 | |
| 45344 | 155613388 | 155614709 | 170624 |
| 45345 | 155794445 | 155795275 | 170624 |
| 45346 | 156472545 | 156473770 | 113249 |
| 45347 | 156476573 | 156477429 | 113249 |
| 45348 | 156485252 | 156487610 | 113249 |
| 45349 | 156526812 | 156527859 | 135077 |
| 45350 | 156568898 | 156571138 | 113263, 135077, 155868 |
| 45351 | 156692562 | 156694424 | 055163, 253653 |
| 45352 | 156713641 | 156714777 | 055163 |
| 45353 | 156716258 | 156719412 | 055163 |
| 45354 | 156727439 | 156728331 | 055163 |
| 45355 | 156906151 | 156907491 | 135074 |
| 45356 | 156911852 | 156912772 | 135074 |
| 45357 | 156937925 | 156939851 | 135074 |
| 45358 | 156947847 | 156949472 | 135074 |
| 45359 | 156974405 | 156978008 | 135074 |
| 45360 | 156981267 | 156982701 | 135074 |
| 45361 | 156986207 | 156986786 | 135074 |
| 45362 | 156990333 | 156992002 | 135074 |
| 45363 | 157001746 | 157003302 | 135074 |
| 45364 | 157158053 | 157158951 | 113272 |
| 45365 | 157170166 | 157171845 | 155858 |
| 45366 | 157229755 | 157230279 | 113282 |
| 45367 | 157249358 | 157249932 | 113282 |
| 45368 | 157272851 | 157273337 | 113282 |
| 45369 | 157284696 | 157286503 | 113282 |
| 45370 | 157297056 | 157299651 | |
| 45371 | 157353223 | 157354099 | |
| 45372 | 157369527 | 157370047 | |
| 45373 | 157375634 | 157376981 | |
| 45374 | 157477852 | 157478606 | |
| 45375 | 157511116 | 157512074 | |
| 45376 | 157580116 | 157581904 | |
| 45377 | 157878233 | 157878972 | 254350 |
| 45378 | 157883582 | 157884712 | |
| 45379 | 157888149 | 157888900 | |
| 45380 | 157890082 | 157891453 | |
| 45381 | 157897623 | 157899305 | |
| 45382 | 157970793 | 157971405 | |
| 45383 | 158081296 | 158081957 | |
| 45384 | 158122310 | 158123616 | 164330 |
| 45385 | 158157841 | 158158564 | 164330 |
| 45386 | 158180915 | 158181772 | 164330 |
| 45387 | 158202340 | 158202924 | 164330 |
| 45388 | 158369695 | 158370844 | 164330 |
| 45389 | 158402807 | 158403472 | 164330 |
| 45390 | 158461114 | 158462148 | 164330 |
| 45391 | 158634604 | 158637075 | 253256, 145860 |
| 45392 | 158689306 | 158691114 | 164332 |
| 45393 | 158890796 | 158891478 | 253298 |
| 45394 | 158937284 | 158939816 | |
| 45395 | 158945016 | 158945765 | |
| 45396 | 159291867 | 159293409 | 253311 |
| 45397 | 159300701 | 159301910 | |
| 45398 | 159337429 | 159340646 | |
| 45399 | 159342704 | 159344183 | 170214 |
| 45400 | 159361042 | 159361530 | 170214 |
| 45401 | 159363660 | 159364091 | 170214 |
| 45402 | 159374627 | 159375700 | 170214 |
| 45403 | 159401993 | 159403058 | |
| 45404 | 159435676 | 159436855 | 113312 |
| 45405 | 159545581 | 159547100 | 170234 |
| 45406 | 159742924 | 159743501 | 135083 |
| 45407 | 159845313 | 159847002 | 164609 |
| 45408 | 159894497 | 159895383 | 253522 |
| 45409 | 162806401 | 162807055 | 250061 |
| 45410 | 162861318 | 162861860 | 250061 |
| 45411 | 162863100 | 162866010 | 113328, 250061 |
| 45412 | 162886220 | 162888056 | 072571, 170584 |
| 45413 | 162916131 | 162917512 | 251018, 072571 |
| 45414 | 162930786 | 162934035 | 038274 |
| 45415 | 162936176 | 162936915 | 038274 |
| 45416 | 163076275 | 163076906 | |
| 45417 | 163187319 | 163187983 | |
| 45418 | 163365665 | 163366010 | |
| 45419 | 163785781 | 163786750 | 241956 |
| 45420 | 165054722 | 165055348 | 253693 |
| 45421 | 166143827 | 166144527 | |
| 45422 | 166146773 | 166147764 | |
| 45423 | 166149295 | 166151822 | |
| 45424 | 166721597 | 166722727 | 145934 |
| 45425 | 166805850 | 166806830 | 145934 |
| 45426 | 166869667 | 166870418 | 145934 |
| 45427 | 167080547 | 167081303 | 254187, 145934 |
| 45428 | 167126516 | 167127232 | 145934 |
| 45429 | 167412308 | 167412952 | 145934 |
| 45430 | 167563150 | 167563857 | 253925, 145934 |
| 45431 | 167590701 | 167591275 | 253925, 145934 |
| 45432 | 167696235 | 167697331 | |
| 45433 | 167714695 | 167720689 | 113645 |
| 45434 | 167722440 | 167723048 | 113645 |
| 45435 | 167728696 | 167731475 | 113645 |
| 45436 | 167731983 | 167733216 | 113645 |
| 45437 | 167735040 | 167736979 | 113645 |
| 45438 | 167739158 | 167740359 | 113645 |
| 45439 | 167740366 | 167745665 | 113645 |
| 45440 | 167751460 | 167753039 | 113645 |
| 45441 | 167755759 | 167756766 | 113645 |
| 45442 | 167776934 | 167777658 | 113645 |
| 45443 | 167780533 | 167781053 | 113645 |
| 45444 | 167789080 | 167790319 | 113645 |
| 45445 | 167791926 | 167793512 | 113645 |
| 45446 | 167912962 | 167914839 | 113643 |
| 45447 | 168004969 | 168007737 | 120137 |
| 45448 | 168018676 | 168019526 | |
| 45449 | 168027283 | 168028220 | |
| 45450 | 168037951 | 168038983 | |
| 45451 | 168051717 | 168052621 | |
| 45452 | 168073993 | 168074412 | |
| 45453 | 168078372 | 168079579 | |
| 45454 | 168108576 | 168109204 | 184347 |
| 45455 | 168304437 | 168305141 | 184347 |
| 45456 | 168416742 | 168417194 | 184347 |
| 45457 | 169009878 | 169011742 | 040275 |
| 45458 | 169577879 | 169578861 | |
| 45459 | 170288226 | 170289862 | 204764 |
| 45460 | 170292250 | 170293179 | 204764 |
| 45461 | 170813543 | 170816194 | 264249, 181163 |
| 45462 | 171164435 | 171164983 | |
| 45463 | 171372007 | 171373223 | 072803 |
| 45464 | 171380249 | 171382748 | 072803 |
| 45465 | 171383060 | 171384230 | 072803 |
| 45466 | 171399671 | 171400150 | 072803 |
| 45467 | 171423847 | 171424477 | 072803 |
| 45468 | 171431653 | 171434880 | 072803 |
| 45469 | 171591246 | 171591950 | 072786 |
| 45470 | 171614020 | 171616232 | 072786 |
| 45471 | 171709459 | 171711970 | 168246 |
| 45472 | 171781510 | 171782537 | 174705 |
| 45473 | 171787916 | 171790217 | 174705 |
| 45474 | 171809872 | 171810830 | 174705 |
| 45475 | 171845204 | 171845708 | 174705 |
| 45476 | 171847068 | 171848019 | 174705 |
| 45477 | 171870304 | 171870989 | 174705 |
| 45478 | 171880506 | 171882074 | 174705 |
| 45479 | 172086715 | 172088214 | 214357 |
| 45480 | 172141005 | 172144739 | |
| 45481 | 172158178 | 172159521 | |
| 45482 | 172190177 | 172210527 | 206741, 120129, 253736, |
| | | | 213386 |
| 45483 | 172215145 | 172217495 | |
| 45484 | 172220715 | 172222020 | |
| 45485 | 172224107 | 172230603 | |
| 45486 | 172232710 | 172235748 | |
| 45487 | 172257197 | 172265009 | 113719 |
| 45488 | 172266648 | 172267382 | 113719 |
| 45489 | 172274718 | 172290262 | 113719 |
| 45490 | 172293839 | 172299592 | 113719 |
| 45491 | 172312390 | 172317015 | 113719 |
| 45492 | 172317672 | 172334295 | 113719 |
| 45493 | 172337012 | 172337435 | 113719 |
| 45494 | 172337563 | 172339400 | 113719 |
| 45495 | 172340072 | 172340937 | 113719 |
| 45496 | 172357536 | 172360359 | 113719 |
| 45497 | 172380186 | 172384946 | 254295, 204758 |
| 45498 | 172385007 | 172387827 | 037241, 204758 |
| 45499 | 172410359 | 172412095 | 113732 |
| 45500 | 172443175 | 172444259 | 113732 |
| 45501 | 172482745 | 172485133 | 164463 |
| 45502 | 172520528 | 172521776 | 164463 |
| 45503 | 172571105 | 172572260 | 113734, 253172 |
| 45504 | 172592178 | 172592854 | |
| 45505 | 172754011 | 172757438 | 113739 |
| 45506 | 172768969 | 172770261 | |
| 45507 | 172877752 | 172883528 | 253968 |
| 45508 | 172899161 | 172900441 | 253141 |
| 45509 | 172924736 | 172927501 | |
| 45510 | 173042593 | 173044324 | 145919 |
| 45511 | 173054965 | 173055587 | |
| 45512 | 173097109 | 173099117 | |
| 45513 | 173115548 | 173117952 | 253428 |
| 45514 | 173146345 | 173147229 | 253686 |
| 45515 | 173148490 | 173149077 | 253686 |
| 45516 | 173189809 | 173196472 | |
| 45517 | 173197774 | 173199085 | |
| 45518 | 173208161 | 173210400 | |
| 45519 | 173215163 | 173219421 | 254211 |
| 45520 | 173229568 | 173230555 | 254211 |
| 45521 | 173233293 | 173234261 | 254211 |
| 45522 | 173250314 | 173252535 | |
| 45523 | 173252834 | 173254483 | |
| 45524 | 173309035 | 173311981 | |
| 45525 | 173314294 | 173320211 | 113742 |
| 45526 | 173359231 | 173360560 | 113742 |
| 45527 | 173380305 | 173381279 | 113742 |
| 45528 | 173395819 | 173397692 | |
| 45529 | 173399409 | 173401187 | 185056 |
| 45530 | 173616514 | 173617161 | 170091 |
| 45531 | 174441598 | 174442617 | |
| 45532 | 174873398 | 174873793 | |
| 45533 | 174894814 | 174895628 | |
| 45534 | 174901927 | 174903245 | |
| 45535 | 174904181 | 174906925 | 164466 |
| 45536 | 174999868 | 175000476 | |
| 45537 | 175281056 | 175282496 | 145920 |
| 45538 | 175294508 | 175295977 | 145920 |
| 45539 | 175316342 | 175317837 | |
| 45540 | 175348900 | 175350164 | 051596, 250820 |
| 45541 | 175779013 | 175779715 | 122203, 250909 |
| 45542 | 175787779 | 175789243 | 122203 |
| 45543 | 175814430 | 175817089 | 146066, 048162, 175414 |
| 45544 | 175842298 | 175844117 | 175416 |
| 45545 | 175873861 | 175876392 | 113194 |
| 45546 | 175893231 | 175893839 | 113194 |
| 45547 | 175928370 | 175928810 | 113194 |
| 45548 | 175960762 | 175965762 | 146083 |
| 45549 | 175969363 | 175971439 | 074276 |
| 45550 | 175972208 | 175973656 | 074276 |
| 45551 | 176073759 | 176075145 | 048140 |
| 45552 | 176432677 | 176434376 | 087206 |
| 45553 | 176448638 | 176450721 | 113761, 087206 |
| 45554 | 176513861 | 176515178 | 160867 |
| 45555 | 176559174 | 176562462 | 165671 |
| 45556 | 176727270 | 176732238 | 169230, 169228, 213347 |
| 45557 | 176752825 | 176753579 | |
| 45558 | 176762253 | 176762717 | 169223 |
| 45559 | 176777857 | 176779694 | 169223 |
| 45560 | 176781060 | 176783040 | |
| 45561 | 176784640 | 176785785 | 169220 |
| 45562 | 176793397 | 176794803 | 169220 |
| 45563 | 176795437 | 176796072 | 169220 |
| 45564 | 176797709 | 176798753 | 169220 |
| 45565 | 176800830 | 176801262 | |
| 45566 | 176804964 | 176806960 | 131183 |
| 45567 | 176835775 | 176836929 | 131187, 198055 |
| 45568 | 176852379 | 176854715 | 198055 |
| 45569 | 176873507 | 176875561 | 246334, 131188 |
| 45570 | 176881303 | 176883762 | 113758, 131188 |
| 45571 | 176921042 | 176921851 | 196923 |
| 45572 | 176924148 | 176925234 | 196923, 248996 |
| 45573 | 176942939 | 176944880 | 183258 |
| 45574 | 176964014 | 176965806 | 146067 |
| 45575 | 176972156 | 176974308 | 146067 |
| 45576 | 176979794 | 176982162 | 272459, 146067 |
| 45577 | 177018956 | 177020053 | 184840 |
| 45578 | 177026765 | 177028099 | 027847 |
| 45579 | 177557178 | 177559049 | 145916 |
| 45580 | 177579792 | 177581246 | 145912 |
| 45581 | 177630869 | 177634010 | 197451 |
| 45582 | 177640023 | 177641379 | 175309 |
| 45583 | 177654704 | 177656052 | 175309 |
| 45584 | 177657379 | 177660498 | 175309 |
| 45585 | 177663593 | 177664193 | |
| 45586 | 177757784 | 177760775 | 050767 |
| 45587 | 177762256 | 177763264 | 050767 |
| 45588 | 177764568 | 177766173 | 050767 |
| 45589 | 177887826 | 177888471 | 050767 |
| 45590 | 178052518 | 178054934 | 113240 |
| 45591 | 178156956 | 178158211 | 169131 |
| 45592 | 178286476 | 178287773 | 178338 |
| 45593 | 178322681 | 178323349 | 198939 |
| 45594 | 178450608 | 178451089 | 234284 |
| 45595 | 178487026 | 178488092 | 177932 |
| 45596 | 178976434 | 178978899 | 176783 |
| 45597 | 179049380 | 179052231 | 169045 |
| 45598 | 179104741 | 179106437 | 225051, 204659, 127022 |
| 45599 | 179107084 | 179108969 | 204659, 225051, 127022 |
| 45600 | 179124643 | 179127839 | 127022 |
| 45601 | 179158986 | 179160946 | 161021 |
| 45602 | 179181634 | 179182567 | 161021 |
| 45603 | 179222498 | 179223935 | 161021, 213316 |
| 45604 | 179228784 | 179234841 | 161011, 161013 |
| 45605 | 179240127 | 179240685 | 161011 |
| 45606 | 179243418 | 179253296 | 161011 |
| 45607 | 179255772 | 179256750 | 161011 |
| 45608 | 179259139 | 179261477 | 161010, 161011 |
| 45609 | 179263317 | 179265291 | 161011, 161010 |
| 45610 | 179265675 | 179267122 | 161010 |
| 45611 | 179267202 | 179269471 | 161010 |
| 45612 | 179285120 | 179286790 | 245317, 161010 |
| 45613 | 179333699 | 179335294 | 197226 |
| 45614 | 179386869 | 179387605 | 113269 |
| 45615 | 179453329 | 179453794 | 113269 |
| 45616 | 179461497 | 179461981 | 113269 |
| 45617 | 179497608 | 179499735 | 113269 |
| 45618 | 179502612 | 179503148 | |
| 45619 | 179516952 | 179517897 | |
| 45620 | 179718143 | 179719930 | 050748 |
| 45621 | 179891277 | 179892044 | |
| 45622 | 179893924 | 179894924 | |
| 45623 | 179921000 | 179922338 | 113300 |
| 45624 | 180228751 | 180230565 | 131446 |
| 45625 | 180234142 | 180236280 | 131446 |
| 45626 | 180236390 | 180238857 | 131446 |
| 45627 | 180256923 | 180259307 | 245060 |
| 45628 | 180287472 | 180289122 | 196670 |
| 45629 | 180600421 | 180601358 | |
| 45630 | 180614397 | 180616286 | |
| 45631 | 180617899 | 180619921 | 250222, 248473 |
| 45632 | 180633521 | 180635333 | |
| 45633 | 180644399 | 180645960 | |
| 45634 | 180648453 | 180651527 | 146063, 264732 |
| 45635 | 180668668 | 180675346 | 264549, 272296, 233937, |
| | | | 204628 |
| 45636 | 180686719 | 180689449 | 248275, 183718, 233937 |

The locations of ODSCs on Chromosome 6 are shown in Table 48.

TABLE 48
ODSC Locations on Chromosome 6

| SEQ ID NO. | Start | End | Overlapping Gene(s) |
|---|---|---|---|
| 45637 | 290777 | 293934 | 112679 |
| 45638 | 385835 | 388940 | |
| 45639 | 421959 | 424457 | |
| 45640 | 692265 | 693619 | 112685 |
| 45641 | 710419 | 711806 | 271727, 272463 |
| 45642 | 743716 | 744149 | 271727 |
| 45643 | 834860 | 838369 | |
| 45644 | 900181 | 902028 | |
| 45645 | 1183156 | 1183839 | |
| 45646 | 1228105 | 1228503 | |
| 45647 | 1311068 | 1312615 | |
| 45648 | 1322253 | 1323587 | |
| 45649 | 1324770 | 1326588 | |
| 45650 | 1334851 | 1337085 | |
| 45651 | 1481445 | 1481945 | |
| 45652 | 1609792 | 1610750 | 54598 |
| 45653 | 1679195 | 1679857 | 112699 |
| 45654 | 1797955 | 1798473 | 112699 |
| 45655 | 1820283 | 1820883 | 112699 |
| 45656 | 1832940 | 1833654 | 112699 |
| 45657 | 2016732 | 2017596 | 112699 |
| 45658 | 2225701 | 2226434 | 112699 |
| 45659 | 2245314 | 2247071 | 250903, 112699 |
| 45660 | 2515152 | 2515821 | |
| 45661 | 2634009 | 2635156 | 164385 |
| 45662 | 2715943 | 2716508 | 145949 |
| 45663 | 2765035 | 2767401 | 124535 |
| 45664 | 2791236 | 2791999 | |
| 45665 | 2838860 | 2842941 | 21355 |
| 45666 | 2846342 | 2847873 | |
| 45667 | 2850887 | 2851508 | |
| 45668 | 2853208 | 2855990 | 266750, 230438 |
| 45669 | 2866543 | 2867300 | 230438 |
| 45670 | 2875081 | 2878206 | 230438 |
| 45671 | 2902376 | 2903857 | 170542 |
| 45672 | 2952683 | 2953298 | 124570 |
| 45673 | 2970824 | 2972649 | 124570 |
| 45674 | 2980034 | 2981055 | |
| 45675 | 2988032 | 2991540 | 124588, 224846, 244041 |
| 45676 | 2998837 | 3001142 | 224846, 124588 |
| 45677 | 3001301 | 3002526 | 124588 |
| 45678 | 3053377 | 3054549 | |
| 45679 | 3063891 | 3064850 | 137275 |
| 45680 | 3068185 | 3070242 | 272277, 137275 |
| 45681 | 3075690 | 3076317 | 137275 |
| 45682 | 3118140 | 3120024 | 137274 |
| 45683 | 3126452 | 3127061 | 137274 |
| 45684 | 3135114 | 3136570 | 137274 |
| 45685 | 3136773 | 3140432 | 228170, 137274 |
| 45686 | 3156747 | 3158799 | 137267 |
| 45687 | 3162280 | 3164003 | |
| 45688 | 3231266 | 3232344 | 180822, 137285 |
| 45689 | 3258931 | 3260573 | 180822 |
| 45690 | 3295659 | 3296845 | 180822, 137266 |
| 45691 | 3324659 | 3325140 | 137266 |
| 45692 | 3334546 | 3335687 | 137266 |
| 45693 | 3340531 | 3341983 | 137266 |
| 45694 | 3356711 | 3357699 | 137266 |
| 45695 | 3363416 | 3365271 | 137266 |
| 45696 | 3366724 | 3368230 | 137266 |
| 45697 | 3372440 | 3374436 | 137266 |
| 45698 | 3376667 | 3377599 | 137266 |
| 45699 | 3377769 | 3378754 | 137266 |
| 45700 | 3380315 | 3384189 | 137266 |
| 45701 | 3389482 | 3391241 | 137266 |
| 45702 | 3403134 | 3404234 | 137266 |
| 45703 | 3405635 | 3407656 | 137266 |
| 45704 | 3409844 | 3411587 | 137266 |
| 45705 | 3418324 | 3421518 | 137266 |
| 45706 | 3454492 | 3458368 | 137266 |
| 45707 | 3517544 | 3518699 | |
| 45708 | 3522119 | 3524353 | |
| 45709 | 3591938 | 3593103 | |
| 45710 | 3742445 | 3744779 | 168994 |
| 45711 | 3747471 | 3753937 | 270504, 168994 |
| 45712 | 3787452 | 3791144 | |
| 45713 | 3796715 | 3799690 | |
| 45714 | 3803168 | 3804909 | |
| 45715 | 3808435 | 3809700 | |
| 45716 | 3825042 | 3827708 | |
| 45717 | 3834851 | 3836612 | 238158 |
| 45718 | 3839089 | 3845037 | 238158 |
| 45719 | 3847602 | 3850624 | 145945, 238158 |
| 45720 | 3869989 | 3871301 | |
| 45721 | 3871745 | 3873657 | |
| 45722 | 3884142 | 3885200 | |
| 45723 | 3909110 | 3915154 | 218472, 260604 |
| 45724 | 3919222 | 3919919 | |
| 45725 | 4007893 | 4009114 | |
| 45726 | 4017544 | 4019200 | 272248, 230648 |
| 45727 | 4020752 | 4022689 | 1 12739, 230648 |
| 45728 | 4058270 | 4060735 | 1 12739, 145975 |
| 45729 | 4066546 | 4067157 | 145975 |
| 45730 | 4125172 | 4127266 | 185689, 198721 |
| 45731 | 4130677 | 4131354 | 185689, 198721 |
| 45732 | 4133987 | 4136849 | 234817, 254821, 198721 |
| 45733 | 4212729 | 4213317 | |
| 45734 | 4271063 | 4271662 | |
| 45735 | 4358677 | 4360291 | |
| 45736 | 4647512 | 4648227 | |
| 45737 | 4775528 | 4777756 | 236336, 153046 |
| 45738 | 4810200 | 4811096 | 153046 |
| 45739 | 4848674 | 4849407 | 153046 |
| 45740 | 4941886 | 4944395 | 153046 |
| 45741 | 5003685 | 5005132 | 124787 |
| 45742 | 5059383 | 5060069 | |
| 45743 | 5061355 | 5061910 | |
| 45744 | 5068191 | 5068917 | |
| 45745 | 5084598 | 5085636 | |
| 45746 | 5115330 | 5115887 | 214113 |
| 45747 | 5118810 | 5119709 | 214113 |
| 45748 | 5121615 | 5122295 | 214113 |
| 45749 | 5190695 | 5191328 | 214113 |
| 45750 | 5205934 | 5206644 | 214113 |
| 45751 | 5260113 | 5262578 | 145982, 214113 |
| 45752 | 5333801 | 5334411 | 145982 |
| 45753 | 5456766 | 5458849 | 269985, 145982 |
| 45754 | 5892431 | 5893251 | |
| 45755 | 6152211 | 6152942 | 124491 |
| 45756 | 6409831 | 6410280 | 216863 |
| 45757 | 6686904 | 6687898 | |
| 45758 | 6711965 | 6714009 | |
| 45759 | 6723922 | 6726651 | |
| 45760 | 6748575 | 6749405 | |
| 45761 | 6810017 | 6812823 | |
| 45762 | 6823683 | 6824686 | |
| 45763 | 6851048 | 6851845 | |
| 45764 | 6856360 | 6857929 | |
| 45765 | 6956707 | 6958758 | |
| 45766 | 6975612 | 6976082 | |
| 45767 | 6998350 | 6998770 | |
| 45768 | 7041681 | 7043869 | |
| 45769 | 7051026 | 7053280 | |
| 45770 | 7058029 | 7059507 | |
| 45771 | 7106306 | 7110177 | 124782 |
| 45772 | 7121683 | 7122349 | 124782 |
| 45773 | 7129207 | 7130877 | 124782 |
| 45774 | 7139758 | 7143507 | 124782 |
| 45775 | 7159768 | 7160950 | 124782 |
| 45776 | 7161032 | 7162012 | 124782 |
| 45777 | 7165803 | 7167680 | 124782 |
| 45778 | 7170980 | 7172227 | 124782 |
| 45779 | 7201047 | 7201840 | 124782 |
| 45780 | 7214311 | 7218167 | 124782 |
| 45781 | 7235856 | 7237303 | 124782 |
| 45782 | 7238921 | 7240238 | 124782 |
| 45783 | 7260795 | 7262795 | |
| 45784 | 7267679 | 7268719 | 124783 |
| 45785 | 7270872 | 7271612 | 124783 |
| 45786 | 7312152 | 7314117 | 124783 |
| 45787 | 7389138 | 7391063 | 124784, 164304 |
| 45788 | 7463920 | 7464478 | |
| 45789 | 7505447 | 7506573 | 219993 |
| 45790 | 7541072 | 7544723 | 096696, 261189 |
| 45791 | 7548549 | 7549402 | 96696 |
| 45792 | 7550423 | 7551326 | 96696 |
| 45793 | 7561177 | 7562405 | 96696 |
| 45794 | 7581275 | 7582162 | 96696 |
| 45795 | 7590050 | 7591146 | 168566 |
| 45796 | 7697307 | 7699277 | |
| 45797 | 7701692 | 7702821 | |
| 45798 | 7899170 | 7899890 | 239264, 259040 |
| 45799 | 7909266 | 7911879 | 239264, 259040 |
| 45800 | 7975093 | 7976159 | 239264, 259040 |
| 45801 | 8047176 | 8048860 | 265818, 259040, 188428 |
| 45802 | 8063559 | 8065882 | 259040, 188428, 265818 |
| 45803 | 8079953 | 8081628 | 124802, 265818 |
| 45804 | 8082619 | 8086073 | 124802, 265818 |
| 45805 | 8101806 | 8103123 | 124802, 265818 |
| 45806 | 8434886 | 8436495 | 124786 |
| 45807 | 8555739 | 8558335 | |
| 45808 | 8633752 | 8634698 | |
| 45809 | 8639998 | 8640968 | |
| 45810 | 8652067 | 8653690 | 251164 |
| 45811 | 8655701 | 8657183 | |
| 45812 | 8664283 | 8665593 | |
| 45813 | 8707689 | 8708981 | |
| 45814 | 8743258 | 8744070 | |
| 45815 | 8905759 | 8907330 | |
| 45816 | 8908639 | 8909310 | |
| 45817 | 9139985 | 9142285 | 225775 |
| 45818 | 9704995 | 9705681 | 181355 |
| 45819 | 10033140 | 10034105 | 181355 |
| 45820 | 10403872 | 10405584 | 137203 |
| 45821 | 10426175 | 10427355 | 228478 |
| 45822 | 10441187 | 10441986 | 229401 |
| 45823 | 10510552 | 10512040 | 237685, 111846 |
| 45824 | 10527943 | 10530253 | 111846 |
| 45825 | 10693666 | 10696181 | 111845, 137434 |
| 45826 | 10722786 | 10723639 | 111843 |
| 45827 | 10734867 | 10735798 | |
| 45828 | 10747304 | 10748687 | 137210, 272162, 153157, |
| | | | 272097 |
| 45829 | 10758839 | 10760828 | 137210, 272162, 153157 |
| 45830 | 10801017 | 10801986 | 111837, 137210, 272162, |
| | | | 153157 |
| 45831 | 10838031 | 10839305 | 111837, 137210, 272162, |
| | | | 153157 |
| 45832 | 11011428 | 11013166 | 197977 |
| 45833 | 11043560 | 11045090 | 230314, 197977 |
| 45834 | 11054582 | 11056374 | 230314 |
| 45835 | 11066809 | 11068096 | 230314 |
| 45836 | 11093037 | 11095971 | 224531 |
| 45837 | 11098193 | 11098657 | 224531 |
| 45838 | 11143269 | 11145223 | |
| 45839 | 11187066 | 11189382 | 111859, 247925 |
| 45840 | 11206334 | 11211888 | 111859, 247925 |
| 45841 | 11226181 | 11226702 | 111859, 247925 |
| 45842 | 11228906 | 11234295 | 111859, 247925 |
| 45843 | 11236460 | 11237127 | 111859, 247925 |
| 45844 | 11262499 | 11264932 | 111859 |
| 45845 | 11275747 | 11276313 | 111859 |
| 45846 | 11317869 | 11319100 | 111859 |
| 45847 | 11341288 | 11347234 | 111859 |
| 45848 | 11350145 | 11352060 | 111859 |
| 45849 | 11362262 | 11369768 | 111859 |
| 45850 | 11392715 | 11394308 | |
| 45851 | 11398152 | 11399747 | |
| 45852 | 11411880 | 11415849 | |
| 45853 | 11423588 | 11425253 | 233656 |
| 45854 | 11425461 | 11427031 | 233656 |
| 45855 | 11428280 | 11430289 | 233656 |
| 45856 | 11431639 | 11436580 | 233656 |
| 45857 | 11472958 | 11474282 | 233656 |
| 45858 | 11535870 | 11536418 | |
| 45859 | 11537042 | 11539072 | 205269 |
| 45860 | 11585573 | 11587243 | |
| 45861 | 11592304 | 11593463 | |
| 45862 | 11598658 | 11599627 | |
| 45863 | 11684982 | 11686675 | |
| 45864 | 11761885 | 11762625 | 111863 |
| 45865 | 11764942 | 11769938 | 111863 |
| 45866 | 11785650 | 11786244 | 111863 |
| 45867 | 11794624 | 11796302 | 111863 |
| 45868 | 11803301 | 11804088 | 111863 |
| 45869 | 11810073 | 11810891 | 234427 |
| 45870 | 11853524 | 11856266 | |
| 45871 | 11867876 | 11869380 | |
| 45872 | 11869835 | 11870536 | |
| 45873 | 12000771 | 12002628 | |
| 45874 | 12007761 | 12013994 | 229896, 095951 |
| 45875 | 12045029 | 12045669 | 95951 |
| 45876 | 12119196 | 12119846 | 95951 |
| 45877 | 12289466 | 12293087 | 78401 |
| 45878 | 12295996 | 12297545 | 78401 |
| 45879 | 12322964 | 12325871 | |
| 45880 | 12331224 | 12333020 | |
| 45881 | 12335912 | 12337033 | |
| 45882 | 12339644 | 12341129 | |
| 45883 | 12342811 | 12344653 | |
| 45884 | 12348615 | 12349851 | |
| 45885 | 12363638 | 12367068 | |
| 45886 | 12368197 | 12369012 | |
| 45887 | 12369542 | 12372693 | |
| 45888 | 12383390 | 12384044 | |
| 45889 | 12472388 | 12475430 | |
| 45890 | 12483485 | 12485252 | |
| 45891 | 12491443 | 12493335 | |
| 45892 | 12538828 | 12539898 | |
| 45893 | 12573019 | 12574248 | |
| 45894 | 12592532 | 12593942 | |
| 45895 | 12958181 | 12958506 | 112137 |
| 45896 | 13256704 | 13257238 | 112137 |
| 45897 | 13269332 | 13269932 | 112137, 145979, 215022 |
| 45898 | 13289841 | 13290775 | 272379, 145979, 215022 |
| 45899 | 13327748 | 13329718 | 145979 |
| 45900 | 13383512 | 13384311 | 145990 |
| 45901 | 13406252 | 13409216 | 145990 |
| 45902 | 13412539 | 13415403 | 145990 |
| 45903 | 13417821 | 13418662 | 145990 |
| 45904 | 13456688 | 13458289 | 145990 |
| 45905 | 13461923 | 13462605 | 145990 |
| 45906 | 13474530 | 13475633 | 187461, 145990 |
| 45907 | 13477045 | 13478069 | 187461, 145990 |
| 45908 | 13479493 | 13482507 | 187461, 145990 |
| 45909 | 13483733 | 13488481 | 237786, 187461, 145990 |
| 45910 | 13548489 | 13549562 | |
| 45911 | 13558718 | 13560029 | |
| 45912 | 13561671 | 13563015 | |
| 45913 | 13566579 | 13567154 | |
| 45914 | 13573229 | 13575995 | 124523 |
| 45915 | 13614218 | 13616392 | 225921, 261071, 268059, |
| | | | 124523 |
| 45916 | 13678048 | 13678979 | 10017 |
| 45917 | 13710195 | 13713076 | 10017 |
| 45918 | 13811460 | 13812556 | 50393 |
| 45919 | 13813499 | 13815592 | 50393 |
| 45920 | 13863821 | 13864526 | |
| 45921 | 14035140 | 14036495 | |
| 45922 | 14074979 | 14076295 | |
| 45923 | 14117503 | 14118329 | 112149 |
| 45924 | 14148099 | 14148540 | |
| 45925 | 14210649 | 14212651 | |
| 45926 | 14311193 | 14311702 | |
| 45927 | 14366739 | 14367609 | |
| 45928 | 14477081 | 14477963 | |
| 45929 | 14560311 | 14560943 | |
| 45930 | 14758523 | 14759873 | |
| 45931 | 14761993 | 14764315 | |
| 45932 | 14889315 | 14890231 | |
| 45933 | 15026967 | 15027636 | 234261 |
| 45934 | 15036910 | 15037553 | 234261 |
| 45935 | 15133152 | 15133593 | |
| 45936 | 15244754 | 15247779 | 008083, 271888 |
| 45937 | 15247954 | 15249549 | 235488, 008083 |
| 45938 | 15256093 | 15256732 | 8083 |
| 45939 | 15266547 | 15267308 | 8083 |
| 45940 | 15274899 | 15275678 | 8083 |
| 45941 | 15282063 | 15282516 | 8083 |
| 45942 | 15284947 | 15285508 | 8083 |
| 45943 | 15330848 | 15331790 | 8083 |
| 45944 | 15396926 | 15397824 | 8083 |
| 45945 | 15431772 | 15432389 | 8083 |
| 45946 | 15504733 | 15505586 | 8083 |
| 45947 | 15662458 | 15663434 | 47579 |
| 45948 | 15755879 | 15756471 | |
| 45949 | 15761086 | 15761854 | |
| 45950 | 15763607 | 15764841 | |
| 45951 | 15897351 | 15897970 | |
| 45952 | 16128817 | 16130133 | 7944 |
| 45953 | 16239159 | 16240206 | 137198 |
| 45954 | 16244732 | 16245822 | 137198 |
| 45955 | 16333739 | 16334198 | 124788 |
| 45956 | 16334895 | 16335910 | 124788 |
| 45957 | 16345558 | 16346230 | 124788 |
| 45958 | 16356085 | 16356780 | 124788 |
| 45959 | 16472003 | 16473079 | 124788 |
| 45960 | 16539126 | 16540229 | 124788 |
| 45961 | 16582379 | 16584455 | 124788 |
| 45962 | 16585548 | 16587367 | 124788 |
| 45963 | 16676916 | 16678186 | 124788 |
| 45964 | 16681792 | 16682211 | 124788 |
| 45965 | 16683275 | 16685206 | 124788 |
| 45966 | 16685339 | 16686018 | 124788 |
| 45967 | 16698521 | 16700908 | 124788 |
| 45968 | 16712332 | 16714165 | 124788 |
| 45969 | 16759243 | 16765312 | 229931, 272341, 124788 |
| 45970 | 16769276 | 16770229 | |
| 45971 | 16819309 | 16820323 | |
| 45972 | 16821685 | 16823066 | |
| 45973 | 16823227 | 16824602 | |
| 45974 | 16910987 | 16911815 | |
| 45975 | 16985948 | 16987187 | |
| 45976 | 16999690 | 17000341 | |
| 45977 | 17042988 | 17043573 | |
| 45978 | 17101897 | 17102501 | 230873 |
| 45979 | 17115974 | 17116509 | 230873 |
| 45980 | 17159457 | 17159965 | |
| 45981 | 17179596 | 17180410 | |
| 45982 | 17278084 | 17279415 | |
| 45983 | 17280863 | 17282536 | 112183 |
| 45984 | 17310553 | 17313377 | |
| 45985 | 17354702 | 17355756 | |
| 45986 | 17392814 | 17395461 | 112186 |
| 45987 | 17397955 | 17398464 | 112186 |
| 45988 | 17405313 | 17406181 | 112186 |
| 45989 | 17472639 | 17473640 | 112186 |
| 45990 | 17492490 | 17493000 | 112186 |
| 45991 | 17584065 | 17584511 | |
| 45992 | 17599815 | 17602955 | 137414 |
| 45993 | 17603178 | 17603644 | 137414 |
| 45994 | 17703038 | 17703993 | 124789 |
| 45995 | 17704595 | 17707919 | 272269, 124789 |
| 45996 | 17864037 | 17867243 | 137177 |
| 45997 | 17868035 | 17868525 | 137177 |
| 45998 | 17905441 | 17909060 | 137177 |
| 45999 | 17929979 | 17931520 | 137177 |
| 46000 | 17986539 | 17989022 | 137177 |
| 46001 | 18034917 | 18035468 | |
| 46002 | 18043265 | 18043935 | |
| 46003 | 18056961 | 18057670 | |
| 46004 | 18081021 | 18082322 | |
| 46005 | 18118422 | 18119154 | |
| 46006 | 18122052 | 18123389 | 187566 |
| 46007 | 18146859 | 18149149 | 137364 |
| 46008 | 18152505 | 18153981 | 137364 |
| 46009 | 18154215 | 18156742 | 165097, 137364 |
| 46010 | 18263101 | 18265836 | 124795 |
| 46011 | 18386462 | 18390589 | 137393 |
| 46012 | 18405745 | 18406830 | 137393 |
| 46013 | 18432027 | 18433527 | 137393 |
| 46014 | 18468344 | 18468850 | 137393 |
| 46015 | 18471267 | 18471882 | |
| 46016 | 18501788 | 18502587 | |
| 46017 | 18584880 | 18585880 | |
| 46018 | 18598892 | 18599587 | |
| 46019 | 18632617 | 18633432 | |
| 46020 | 19804699 | 19806106 | 228412, 226786 |
| 46021 | 20126856 | 20127779 | 172197 |
| 46022 | 20129498 | 20131720 | 172197 |
| 46023 | 20168912 | 20169929 | 172197 |
| 46024 | 20181165 | 20181572 | 172197 |
| 46025 | 20200754 | 20203627 | 172197 |
| 46026 | 20212096 | 20213858 | 227803, 172197 |
| 46027 | 20400731 | 20405168 | 112242 |
| 46028 | 20424779 | 20425340 | 112242 |
| 46029 | 20442622 | 20443454 | 112242 |
| 46030 | 20447759 | 20448469 | 112242 |
| 46031 | 20448925 | 20449548 | 112242 |
| 46032 | 20534075 | 20535570 | 145996 |
| 46033 | 20542981 | 20543659 | 145996 |
| 46034 | 20631156 | 20631571 | 145996 |
| 46035 | 20773955 | 20774485 | 233848, 145996 |
| 46036 | 20865990 | 20868237 | 145996 |
| 46037 | 20875570 | 20879092 | 145996 |
| 46038 | 20952870 | 20953387 | 145996 |
| 46039 | 21022472 | 21023873 | 145996 |
| 46040 | 21043831 | 21047007 | 145996 |
| 46041 | 21061961 | 21062998 | 145996 |
| 46042 | 21103809 | 21104812 | 145996 |
| 46043 | 21109765 | 21110747 | 145996 |
| 46044 | 21110894 | 21112170 | 145996 |
| 46045 | 21138177 | 21139825 | 145996 |
| 46046 | 21141223 | 21144458 | 145996 |
| 46047 | 21149596 | 21150323 | 145996 |
| 46048 | 21306114 | 21306463 | |
| 46049 | 21587016 | 21590463 | |
| 46050 | 21593595 | 21597645 | 124766 |
| 46051 | 21731673 | 21732516 | 272168 |
| 46052 | 21738485 | 21739360 | 272168 |
| 46053 | 22236017 | 22238015 | |
| 46054 | 22247977 | 22250438 | |
| 46055 | 22250600 | 22252489 | |
| 46056 | 22366340 | 22367068 | |
| 46057 | 22467731 | 22468355 | |
| 46058 | 22827189 | 22829856 | 233358 |
| 46059 | 22880908 | 22882084 | 233358 |
| 46060 | 22888187 | 22889129 | 233358 |
| 46061 | 23414137 | 23414864 | |
| 46062 | 23435674 | 23437042 | |
| 46063 | 23560064 | 23560830 | |
| 46064 | 23584279 | 23584977 | |
| 46065 | 23890485 | 23891447 | |
| 46066 | 24283061 | 24284160 | 146038 |
| 46067 | 24341082 | 24342145 | 146038 |
| 46068 | 24382082 | 24384080 | |
| 46069 | 24385732 | 24386379 | |
| 46070 | 24402913 | 24404243 | 124532 |
| 46071 | 24488621 | 24490562 | 112293 |
| 46072 | 24494497 | 24496151 | 112294, 112293 |
| 46073 | 24504588 | 24505993 | 112294 |
| 46074 | 24665938 | 24669772 | 112304, 111802 |
| 46075 | 24672924 | 24673538 | 112304 |
| 46076 | 24718802 | 24722779 | 112308 |
| 46077 | 24743320 | 24744475 | 224164 |
| 46078 | 24774680 | 24776278 | 112312 |
| 46079 | 25006592 | 25007776 | 111913 |
| 46080 | 25083882 | 25084819 | 168405 |
| 46081 | 25138278 | 25140049 | 168405 |
| 46082 | 25278744 | 25281350 | 79691 |
| 46083 | 25366945 | 25367583 | 79691 |
| 46084 | 25369742 | 25370434 | 79691 |
| 46085 | 25659781 | 25661941 | 79689 |
| 46086 | 25755515 | 25757722 | 146039 |
| 46087 | 25770179 | 25770681 | 146039 |
| 46088 | 25831691 | 25832540 | 124568 |
| 46089 | 25874346 | 25874713 | 124564 |
| 46090 | 25875016 | 25877292 | 124564 |
| 46091 | 25880784 | 25881558 | 124564 |
| 46092 | 25881576 | 25882968 | 242387, 124564 |
| 46093 | 25883570 | 25884826 | |
| 46094 | 25899359 | 25901603 | |
| 46095 | 25903681 | 25904201 | |
| 46096 | 25910360 | 25910999 | |
| 46097 | 25927384 | 25932433 | 112337 |
| 46098 | 25968862 | 25969867 | 112343 |
| 46099 | 25970725 | 25972666 | 112343 |
| 46100 | 25991799 | 25993711 | 272462, 272558 |
| 46101 | 26016207 | 26018144 | 224447, 124610 |
| 46102 | 26020500 | 26022717 | 196176, 198366 |
| 46103 | 26025237 | 26028046 | 124529 |
| 46104 | 26031948 | 26032751 | 124693 |
| 46105 | 26033464 | 26034473 | 137259 |
| 46106 | 26043897 | 26045529 | 234816 |
| 46107 | 26054476 | 26057647 | 187837 |
| 46108 | 26066011 | 26066676 | |
| 46109 | 26087213 | 26088946 | 10704 |
| 46110 | 26102877 | 26105826 | 197061 |
| 46111 | 26119984 | 26126480 | 180573, 180596 |
| 46112 | 26155578 | 26160079 | 168298, 158373 |
| 46113 | 26188090 | 26189665 | 188987 |
| 46114 | 26204250 | 26206821 | 198518 |
| 46115 | 26216483 | 26217982 | 168274, 187990 |
| 46116 | 26284662 | 26287250 | 158406 |
| 46117 | 26305299 | 26306083 | |
| 46118 | 26311018 | 26312374 | |
| 46119 | 26312546 | 26314117 | |
| 46120 | 26322674 | 26323729 | |
| 46121 | 26330247 | 26330938 | |
| 46122 | 26331415 | 26332652 | |
| 46123 | 26351150 | 26352782 | |
| 46124 | 26383186 | 26384174 | 124508 |
| 46125 | 26388185 | 26389165 | 124508 |
| 46126 | 26421497 | 26422335 | 124549 |
| 46127 | 26432888 | 26433412 | |
| 46128 | 26440307 | 26441198 | 111801 |
| 46129 | 26457302 | 26458890 | 112763 |
| 46130 | 26515064 | 26515659 | |
| 46131 | 26520326 | 26521869 | |
| 46132 | 26532736 | 26533855 | |
| 46133 | 26536839 | 26539571 | 182952 |
| 46134 | 26544504 | 26545156 | 182952 |
| 46135 | 26553414 | 26555886 | |
| 46136 | 26556511 | 26557453 | |
| 46137 | 26571675 | 26573250 | |
| 46138 | 26596050 | 26598257 | 146109 |
| 46139 | 26986833 | 26988547 | 238621, 224843 |
| 46140 | 27112850 | 27114377 | 197903 |
| 46141 | 27125173 | 27126421 | |
| 46142 | 27129603 | 27130512 | |
| 46143 | 27144626 | 27146454 | |
| 46144 | 27155629 | 27156994 | |
| 46145 | 27247710 | 27248359 | |
| 46146 | 27356262 | 27357020 | 124613 |
| 46147 | 27440094 | 27441485 | 96654 |
| 46148 | 27446251 | 27447802 | |
| 46149 | 27463318 | 27464061 | |
| 46150 | 27470445 | 27471860 | |
| 46151 | 27473156 | 27473969 | |
| 46152 | 27486042 | 27487645 | |
| 46153 | 27559338 | 27559913 | |
| 46154 | 27560101 | 27560938 | |
| 46155 | 27636089 | 27636879 | |
| 46156 | 27652012 | 27652767 | |
| 46157 | 27655704 | 27656548 | |
| 46158 | 27719927 | 27721539 | |
| 46159 | 27758775 | 27761117 | |
| 46160 | 27763342 | 27764097 | |
| 46161 | 27792054 | 27792743 | 197238 |
| 46162 | 27797460 | 27799234 | 197914 |
| 46163 | 27804016 | 27807035 | 184348, 233822 |
| 46164 | 27807290 | 27807822 | 233822 |
| 46165 | 27839638 | 27841787 | 198558, 182572 |
| 46166 | 27862844 | 27863777 | |
| 46167 | 27869962 | 27871576 | |
| 46168 | 27884994 | 27885601 | |
| 46169 | 28047857 | 28049557 | 197279, 272009 |
| 46170 | 28071990 | 28073354 | |
| 46171 | 28103822 | 28105846 | 261839, 269293 |
| 46172 | 28109233 | 28110546 | 198315 |
| 46173 | 28186136 | 28187245 | 176933 |
| 46174 | 28192242 | 28193932 | 137185 |
| 46175 | 28219321 | 28221094 | 187626 |
| 46176 | 28234292 | 28236308 | 197062 |
| 46177 | 28248920 | 28249698 | 137338 |
| 46178 | 28302453 | 28305166 | 235109 |
| 46179 | 28317183 | 28318708 | 189298, 235109 |
| 46180 | 28366809 | 28368011 | 158691 |
| 46181 | 28410842 | 28411596 | 187987 |
| 46182 | 28510329 | 28511236 | |
| 46183 | 28521101 | 28523158 | |
| 46184 | 28554195 | 28555839 | 246350, 232040 |
| 46185 | 28574641 | 28575390 | 232040 |
| 46186 | 28696808 | 28697734 | |
| 46187 | 28784710 | 28785265 | |
| 46188 | 28805116 | 28807044 | 225173 |
| 46189 | 28830904 | 28832308 | 225595 |
| 46190 | 28848861 | 28849509 | |
| 46191 | 28862858 | 28865715 | 224157 |
| 46192 | 28889179 | 28892650 | 204713 |
| 46193 | 28907852 | 28909797 | |
| 46194 | 28920738 | 28922023 | |
| 46195 | 28948785 | 28951107 | |
| 46196 | 28952725 | 28954178 | 227214 |
| 46197 | 29184196 | 29184786 | |
| 46198 | 29200386 | 29201027 | 232505 |
| 46199 | 29495690 | 29496696 | |
| 46200 | 29526918 | 29528162 | 213886, 204681 |
| 46201 | 29528642 | 29529576 | 204681 |
| 46202 | 29617101 | 29618170 | |
| 46203 | 29690729 | 29691589 | 225864, 204642 |
| 46204 | 29716320 | 29717197 | 214922, 273340 |
| 46205 | 29719763 | 29721340 | 272236 |
| 46206 | 29732622 | 29733047 | |
| 46207 | 29759378 | 29760923 | 176998, 181126 |
| 46208 | 29812247 | 29813738 | |
| 46209 | 29854512 | 29856389 | 230521, 206341 |
| 46210 | 29875291 | 29876680 | 235963 |
| 46211 | 29877583 | 29878224 | |
| 46212 | 29892054 | 29895167 | 230795, 227262 |
| 46213 | 29909463 | 29911195 | 227766, 206503 |
| 46214 | 29931929 | 29936268 | |
| 46215 | 29943841 | 29945597 | 204625 |
| 46216 | 29973833 | 29974516 | 204622, 237669, 204623 |
| 46217 | 30001057 | 30002341 | 204623 |
| 46218 | 30012274 | 30012729 | 204623 |
| 46219 | 30027933 | 30030189 | 204623, 066379 |
| 46220 | 30033936 | 30035774 | 204619 |
| 46221 | 30069635 | 30070485 | |
| 46222 | 30079987 | 30081958 | 204616, 231226 |
| 46223 | 30098430 | 30099293 | |
| 46224 | 30103302 | 30104153 | 204614 |
| 46225 | 30122414 | 30125097 | 204613 |
| 46226 | 30130109 | 30132491 | 204610 |
| 46227 | 30168109 | 30169433 | 234127 |
| 46228 | 30173388 | 30175908 | 234127 |
| 46229 | 30179599 | 30182678 | 234127 |
| 46230 | 30293390 | 30295735 | 204599, 231074, 270604 |
| 46231 | 30312384 | 30313660 | 241370, 248167 |
| 46232 | 30433842 | 30434648 | 229068 |
| 46233 | 30456655 | 30458187 | 204592 |
| 46234 | 30483435 | 30484776 | 235781 |
| 46235 | 30495113 | 30498192 | |
| 46236 | 30501161 | 30502215 | |
| 46237 | 30523186 | 30526000 | 204576, 204590 |
| 46238 | 30530567 | 30531149 | 204576 |
| 46239 | 30538111 | 30540191 | 204574 |
| 46240 | 30582077 | 30586770 | 204568, 222894, 204569 |
| 46241 | 30594301 | 30595540 | 137343 |
| 46242 | 30614670 | 30616239 | 204564 |
| 46243 | 30640018 | 30641217 | 204560 |
| 46244 | 30646579 | 30648314 | 146112 |
| 46245 | 30649757 | 30652015 | 146112 |
| 46246 | 30653777 | 30656497 | 137404, 146112 |
| 46247 | 30663017 | 30663541 | |
| 46248 | 30677161 | 30677692 | 137337, 224328 |
| 46249 | 30684406 | 30685931 | 137337 |
| 46250 | 30687035 | 30689971 | 196230 |
| 46251 | 30707548 | 30712792 | 272273, 137331, 137312 |
| 46252 | 30726522 | 30727919 | |
| 46253 | 30730935 | 30732529 | |
| 46254 | 30736227 | 30739232 | 228022 |
| 46255 | 30748588 | 30751039 | 228022 |
| 46256 | 30795492 | 30796792 | 214894 |
| 46257 | 30874956 | 30876630 | 137411, 213780 |
| 46258 | 30880894 | 30883565 | 213780, 137411 |
| 46259 | 30894291 | 30895001 | |
| 46260 | 30910262 | 30910890 | 168631 |
| 46261 | 30922495 | 30923020 | 233529 |
| 46262 | 30928648 | 30929145 | |
| 46263 | 31090382 | 31091184 | 204540 |
| 46264 | 31125180 | 31126498 | 137310, 204536 |
| 46265 | 31164692 | 31166631 | 206344, 272501 |
| 46266 | 31169803 | 31173077 | 206344 |
| 46267 | 31239158 | 31240406 | 204525 |
| 46268 | 31324159 | 31325332 | 271581, 234745 |
| 46269 | 31334506 | 31335112 | 228432 |
| 46270 | 31367283 | 31371708 | 199332, 206337, 204520 |
| 46271 | 31430031 | 31431521 | 233902, 206337 |
| 46272 | 31508695 | 31510773 | 234006, 265236, 198563, |
| | | | 254870 |
| 46273 | 31514363 | 31516046 | 204498, 254870, 213760 |
| 46274 | 31528009 | 31528666 | |
| 46275 | 31545874 | 31546924 | 232810 |
| 46276 | 31548536 | 31549590 | 227507 |
| 46277 | 31587651 | 31589814 | 204469 |
| 46278 | 31619145 | 31621688 | 204444, 204463 |
| 46279 | 31627121 | 31628761 | 227198, 204439 |
| 46280 | 31631850 | 31634848 | 204435, 263020, 204438 |
| 46281 | 31648423 | 31649488 | 204428 |
| 46282 | 31650429 | 31651200 | 204428 |
| 46283 | 31658022 | 31658542 | 204422, 204427 |
| 46284 | 31669965 | 31671943 | 204427, 204422 |
| 46285 | 31696954 | 31698177 | 213722 |
| 46286 | 31701734 | 31704610 | 213719 |
| 46287 | 31705721 | 31708045 | 204410, 255152, 213719 |
| 46288 | 31743746 | 31745456 | 204394, 204396 |
| 46289 | 31762122 | 31764168 | 204394 |
| 46290 | 31773813 | 31775212 | 204392 |
| 46291 | 31781335 | 31783691 | 204389, 204390 |
| 46292 | 31788850 | 31791103 | |
| 46293 | 31794257 | 31795880 | 204388 |
| 46294 | 31801657 | 31804983 | 204387, 201823, 201754 |
| 46295 | 31829784 | 31831066 | 204385, 204386 |
| 46296 | 31864538 | 31866387 | 166278, 204371 |
| 46297 | 31869310 | 31871024 | 204366, 166278 |
| 46298 | 31878974 | 31879526 | 166278 |
| 46299 | 31894482 | 31897503 | 244255, 243649, 166278 |
| 46300 | 31907916 | 31908501 | 244255, 243649, 166278 |
| 46301 | 31913093 | 31917033 | 166278, 244255, 243649 |
| 46302 | 31925812 | 31927755 | 204351, 204356 |
| 46303 | 31938329 | 31941479 | 204344, 204348 |
| 46304 | 31946687 | 31948004 | 204344 |
| 46305 | 32094837 | 32096410 | 213676 |
| 46306 | 32097369 | 32098455 | 204315 |
| 46307 | 32121464 | 32122783 | 258388, 204314, 221988 |
| 46308 | 32142924 | 32144770 | 204310 |
| 46309 | 32144779 | 32146995 | 204308, 204310 |
| 46310 | 32156910 | 32159252 | 213654, 204304 |
| 46311 | 32162709 | 32164861 | 213654, 204301 |
| 46312 | 32394977 | 32395465 | |
| 46313 | 32764657 | 32765395 | |
| 46314 | 32805371 | 32806906 | 250264, 204267 |
| 46315 | 32811000 | 32812689 | 240065, 204261, 204264 |
| 46316 | 32820369 | 32822870 | 168394, 240065 |
| 46317 | 32828767 | 32829686 | |
| 46318 | 32861527 | 32863224 | |
| 46319 | 32920750 | 32921402 | 248993, 204257 |
| 46320 | 32934739 | 32942568 | 263756, 204256, 223837, |
| | | | 204257 |
| 46321 | 33020891 | 33021511 | |
| 46322 | 33129026 | 33130070 | |
| 46323 | 33162964 | 33163822 | 204231 |
| 46324 | 33167177 | 33169462 | 202441, 112473, 204231 |
| 46325 | 33172170 | 33173615 | 204228, 112473 |
| 46326 | 33174957 | 33176912 | 204227, 199036 |
| 46327 | 33215944 | 33218213 | 232940, 223501 |
| 46328 | 33238621 | 33241035 | 231500, 223501 |
| 46329 | 33256017 | 33258425 | 204220, 227057 |
| 46330 | 33264568 | 33268334 | 231925, 237441, 204220 |
| 46331 | 33280158 | 33282673 | 236104, 231925 |
| 46332 | 33285206 | 33285890 | 236104 |
| 46333 | 33287248 | 33289395 | 204209 |
| 46334 | 33289617 | 33291568 | 204209 |
| 46335 | 33358312 | 33359635 | 237649 |
| 46336 | 33377350 | 33379521 | 112511, 237649 |
| 46337 | 33384872 | 33387098 | 112514 |
| 46338 | 33392681 | 33394217 | 197283 |
| 46339 | 33398066 | 33398608 | 197283 |
| 46340 | 33421202 | 33422942 | 213588, 197283 |
| 46341 | 33538470 | 33539694 | |
| 46342 | 33547072 | 33548444 | 30110 |
| 46343 | 33556828 | 33559726 | 197251 |
| 46344 | 33640388 | 33641586 | 96433 |
| 46345 | 33684077 | 33685507 | |
| 46346 | 33688071 | 33689149 | |
| 46347 | 33706480 | 33709168 | 161896 |
| 46348 | 33711513 | 33715620 | 161896 |
| 46349 | 33717959 | 33719400 | |
| 46350 | 33728628 | 33730539 | |
| 46351 | 33755207 | 33757749 | 161904 |
| 46352 | 33807709 | 33808529 | |
| 46353 | 33874112 | 33876266 | 233183 |
| 46354 | 34163926 | 34164766 | |
| 46355 | 34191495 | 34192842 | |
| 46356 | 34202920 | 34206403 | 137309 |
| 46357 | 34210307 | 34212720 | 137309 |
| 46358 | 34214690 | 34217440 | 186577 |
| 46359 | 34273901 | 34274524 | 272325, 270800 |
| 46360 | 34358718 | 34361200 | 272325, 270800 |
| 46361 | 34382224 | 34383541 | 270800 |
| 46362 | 34392886 | 34394262 | 270800, 124614 |
| 46363 | 34430173 | 34430714 | |
| 46364 | 34494584 | 34495524 | 124507 |
| 46365 | 34570121 | 34571000 | 196821 |
| 46366 | 34577263 | 34578861 | 196821 |
| 46367 | 34623797 | 34626353 | 196821 |
| 46368 | 34626510 | 34627431 | 196821 |
| 46369 | 34631490 | 34633093 | 196821 |
| 46370 | 34635066 | 34635815 | 196821 |
| 46371 | 34640594 | 34642853 | 196821 |
| 46372 | 34648192 | 34649810 | 196821 |
| 46373 | 34662006 | 34665627 | 272288, 196821 |
| 46374 | 34707389 | 34709265 | |
| 46375 | 34709822 | 34710453 | |
| 46376 | 34710919 | 34711743 | |
| 46377 | 34724160 | 34726116 | 124562 |
| 46378 | 34758683 | 34761680 | 65060 |
| 46379 | 34763575 | 34764202 | 65060 |
| 46380 | 34778503 | 34779969 | 65060 |
| 46381 | 34854988 | 34856503 | 64995 |
| 46382 | 34856680 | 34858006 | 64999 |
| 46383 | 34869405 | 34870416 | 64999 |
| 46384 | 34889872 | 34890458 | 64999 |
| 46385 | 34921025 | 34922434 | 64999 |
| 46386 | 34984016 | 34985719 | 64999 |
| 46387 | 35129499 | 35130689 | |
| 46388 | 35132564 | 35134793 | |
| 46389 | 35152434 | 35154065 | |
| 46390 | 35211000 | 35212097 | 146197 |
| 46391 | 35226629 | 35228833 | 65029 |
| 46392 | 35230343 | 35230716 | 65029 |
| 46393 | 35232349 | 35233770 | 65029 |
| 46394 | 35309721 | 35312052 | 112033 |
| 46395 | 35326906 | 35329648 | 112033 |
| 46396 | 35335226 | 35340166 | 112033 |
| 46397 | 35358443 | 35362485 | 112033 |
| 46398 | 35367525 | 35371539 | 112033 |
| 46399 | 35382385 | 35384899 | 112033 |
| 46400 | 35419970 | 35420884 | 112039 |
| 46401 | 35435644 | 35437531 | 198755 |
| 46402 | 35463057 | 35465959 | 112041, 007866 |
| 46403 | 35568636 | 35571977 | 96060 |
| 46404 | 35655294 | 35657327 | 96060 |
| 46405 | 35689599 | 35690967 | 96060 |
| 46406 | 35691041 | 35692476 | 96060 |
| 46407 | 35692631 | 35698086 | 96060 |
| 46408 | 35698492 | 35701071 | |
| 46409 | 35707348 | 35708258 | 157343 |
| 46410 | 35799147 | 35799798 | 197753 |
| 46411 | 35886841 | 35889638 | 96063 |
| 46412 | 35940952 | 35942458 | 112053 |
| 46413 | 35995055 | 35997704 | 112062 |
| 46414 | 36032544 | 36033098 | 112062 |
| 46415 | 36043346 | 36043929 | 112062 |
| 46416 | 36061862 | 36064774 | 112062 |
| 46417 | 36163445 | 36165249 | 096070, 246982 |
| 46418 | 36354108 | 36355002 | 224666, 010030 |
| 46419 | 36381808 | 36383016 | 179165 |
| 46420 | 36390656 | 36391589 | 179165 |
| 46421 | 36408388 | 36412004 | 112078, 179165 |
| 46422 | 36482167 | 36483136 | 112079 |
| 46423 | 36490778 | 36492318 | 112079 |
| 46424 | 36514071 | 36515504 | 112079 |
| 46425 | 36545197 | 36545632 | |
| 46426 | 36560889 | 36563666 | 112081 |
| 46427 | 36576787 | 36577399 | |
| 46428 | 36584068 | 36585468 | |
| 46429 | 36591066 | 36592445 | |
| 46430 | 36609164 | 36609742 | |
| 46431 | 36610125 | 36612257 | |
| 46432 | 36633812 | 36635946 | |
| 46433 | 36643507 | 36649261 | 124762 |
| 46434 | 36649355 | 36652641 | 124762 |
| 46435 | 36655916 | 36657009 | |
| 46436 | 36797624 | 36798265 | 124772 |
| 46437 | 36841326 | 36843270 | 137168, 198663 |
| 46438 | 36853176 | 36854572 | 198663 |
| 46439 | 36881541 | 36882510 | 198663 |
| 46440 | 36887670 | 36888434 | 198663 |
| 46441 | 36952626 | 36954927 | 137409 |
| 46442 | 36990998 | 36991894 | 146192 |
| 46443 | 37001073 | 37002072 | |
| 46444 | 37023619 | 37028306 | |
| 46445 | 37028963 | 37030040 | |
| 46446 | 37050667 | 37052065 | |
| 46447 | 37069633 | 37072067 | |
| 46448 | 37095487 | 37097311 | |
| 46449 | 37097556 | 37100015 | |
| 46450 | 37101337 | 37103574 | |
| 46451 | 37111518 | 37113203 | |
| 46452 | 37114317 | 37114976 | |
| 46453 | 37115839 | 37116594 | |
| 46454 | 37123030 | 37123864 | |
| 46455 | 37136427 | 37140551 | 137193 |
| 46456 | 37172101 | 37172733 | |
| 46457 | 37207126 | 37213726 | 172738 |
| 46458 | 37225192 | 37227743 | 065491, 172738 |
| 46459 | 37251549 | 37252057 | 65491 |
| 46460 | 37400224 | 37401928 | 137200 |
| 46461 | 37441127 | 37441766 | 137200 |
| 46462 | 37466836 | 37467802 | 198937 |
| 46463 | 37483582 | 37485309 | 204110 |
| 46464 | 37587890 | 37588974 | |
| 46465 | 37664510 | 37666417 | 112139 |
| 46466 | 37685507 | 37686172 | |
| 46467 | 37709585 | 37710370 | |
| 46468 | 37729596 | 37730314 | |
| 46469 | 37758827 | 37759745 | |
| 46470 | 37762485 | 37762974 | |
| 46471 | 37766430 | 37768000 | |
| 46472 | 37785685 | 37789006 | 156639, 225945 |
| 46473 | 37856872 | 37858035 | 156639 |
| 46474 | 38046299 | 38047447 | 156639 |
| 46475 | 38062694 | 38063276 | 156639 |
| 46476 | 38099241 | 38099877 | 156639 |
| 46477 | 38229646 | 38230395 | 183826 |
| 46478 | 38372051 | 38373284 | 183826 |
| 46479 | 38606754 | 38608374 | 183826 |
| 46480 | 38669798 | 38671317 | 124767 |
| 46481 | 38813701 | 38815008 | 124721 |
| 46482 | 38874887 | 38875542 | 124721 |
| 46483 | 38877003 | 38877702 | 124721 |
| 46484 | 38942336 | 38942845 | 124721 |
| 46485 | 38943289 | 38943911 | 124721 |
| 46486 | 39082102 | 39083675 | 112167 |
| 46487 | 39152683 | 39153820 | |
| 46488 | 39173717 | 39174684 | 164626 |
| 46489 | 39181726 | 39183797 | 164626 |
| 46490 | 39192021 | 39194160 | 164626 |
| 46491 | 39196623 | 39197715 | 164626 |
| 46492 | 39246734 | 39249952 | |
| 46493 | 39261410 | 39262911 | |
| 46494 | 39342911 | 39343634 | 164627 |
| 46495 | 39674544 | 39677186 | 164627 |
| 46496 | 39692466 | 39693721 | 164627 |
| 46497 | 39887837 | 39888287 | 124615 |
| 46498 | 39901195 | 39902434 | 124615 |
| 46499 | 39906623 | 39907382 | |
| 46500 | 39915493 | 39916588 | |
| 46501 | 39948698 | 39949732 | |
| 46502 | 40493707 | 40494370 | 156564 |
| 46503 | 40495364 | 40496318 | 156564 |
| 46504 | 40555159 | 40556047 | 156564 |
| 46505 | 40682346 | 40683238 | 237947 |
| 46506 | 40689837 | 40691146 | |
| 46507 | 40786654 | 40787955 | |
| 46508 | 40874326 | 40875688 | |
| 46509 | 40924841 | 40931776 | |
| 46510 | 40963671 | 40966785 | |
| 46511 | 40967124 | 40967787 | |
| 46512 | 40980670 | 40981998 | |
| 46513 | 41004198 | 41009544 | 124602, 124596 |
| 46514 | 41013563 | 41014269 | 124596 |
| 46515 | 41031447 | 41034055 | 124701, 124596 |
| 46516 | 41038755 | 41042106 | 001167, 124596 |
| 46517 | 41336490 | 41337151 | |
| 46518 | 41395111 | 41396299 | |
| 46519 | 41417278 | 41418461 | |
| 46520 | 41513197 | 41515965 | 137166, 234753 |
| 46521 | 41526060 | 41526588 | 137166 |
| 46522 | 41536216 | 41536877 | 137166 |
| 46523 | 41545699 | 41547239 | 137166 |
| 46524 | 41552512 | 41553233 | 137166 |
| 46525 | 41570254 | 41571447 | |
| 46526 | 41649657 | 41650406 | |
| 46527 | 41680251 | 41681747 | 112561 |
| 46528 | 41701744 | 41703038 | 231 102, 112561 |
| 46529 | 41703130 | 41703572 | 112561 |
| 46530 | 41746922 | 41749105 | 124593, 137218 |
| 46531 | 41753879 | 41756122 | 214736, 137218, 124593 |
| 46532 | 41862406 | 41864355 | 164663 |
| 46533 | 41885126 | 41886218 | 207371, 124641 |
| 46534 | 41887591 | 41889995 | 112578, 124641 |
| 46535 | 41908883 | 41909982 | 112576 |
| 46536 | 42012976 | 42013859 | 112576 |
| 46537 | 42015675 | 42017002 | 112576 |
| 46538 | 42017720 | 42019003 | 137413, 112576 |
| 46539 | 42055912 | 42056319 | |
| 46540 | 42084888 | 42086953 | 188112 |
| 46541 | 42134058 | 42135185 | 48545 |
| 46542 | 42166854 | 42169412 | |
| 46543 | 42171457 | 42173803 | |
| 46544 | 42178363 | 42179001 | 48544 |
| 46545 | 42184635 | 42186051 | 48544 |
| 46546 | 42191751 | 42196056 | 124496 |
| 46547 | 42531267 | 42533225 | 24048 |
| 46548 | 42688490 | 42689130 | 112619 |
| 46549 | 42712718 | 42715771 | 124659 |
| 46550 | 42748757 | 42752921 | 112624 |
| 46551 | 42812271 | 42813769 | 112624 |
| 46552 | 42844864 | 42846284 | |
| 46553 | 42846371 | 42848654 | 146223 |
| 46554 | 42857647 | 42859014 | 221821, 146223 |
| 46555 | 42896172 | 42898139 | 137161 |
| 46556 | 42907141 | 42907717 | |
| 46557 | 42938536 | 42939673 | 124587 |
| 46558 | 42945617 | 42947598 | 124587 |
| 46559 | 42980762 | 42981842 | 124733 |
| 46560 | 42988468 | 42990048 | 124541, 124702 |
| 46561 | 43026461 | 43028808 | 112651, 137171 |
| 46562 | 43137765 | 43141067 | 112658 |
| 46563 | 43141092 | 43142747 | 112658 |
| 46564 | 43149180 | 43150559 | 112659, 112658 |
| 46565 | 43196323 | 43197514 | 112667 |
| 46566 | 43214795 | 43215609 | 146216 |
| 46567 | 43234705 | 43239223 | 146216 |
| 46568 | 43241572 | 43247558 | 146216 |
| 46569 | 43254682 | 43255998 | 146216 |
| 46570 | 43258169 | 43259307 | |
| 46571 | 43265162 | 43267915 | 146215, 137204 |
| 46572 | 43273648 | 43274645 | 146215 |
| 46573 | 43275403 | 43277250 | 146215, 171467 |
| 46574 | 43296148 | 43297272 | 220614, 171467 |
| 46575 | 43336062 | 43337953 | 171467 |
| 46576 | 43394897 | 43396416 | 124574 |
| 46577 | 43404236 | 43404920 | 124574 |
| 46578 | 43445010 | 43446235 | 137221 |
| 46579 | 43449043 | 43450106 | 137221 |
| 46580 | 43477835 | 43478757 | 204052, 171453 |
| 46581 | 43483749 | 43485752 | 137207, 171453 |
| 46582 | 43542770 | 43544776 | 170734, 124571 |
| 46583 | 43593552 | 43598505 | 124688, 172432 |
| 46584 | 43603001 | 43604588 | 124688 |
| 46585 | 43654583 | 43655835 | 96080 |
| 46586 | 43736904 | 43742962 | 112715, 272114 |
| 46587 | 43765531 | 43769996 | |
| 46588 | 43770502 | 43775854 | |
| 46589 | 43778818 | 43779456 | |
| 46590 | 43790967 | 43792762 | |
| 46591 | 43805912 | 43806992 | |
| 46592 | 43823777 | 43824718 | |
| 46593 | 43878450 | 43886912 | |
| 46594 | 43958494 | 43959093 | |
| 46595 | 43960636 | 43961268 | |
| 46596 | 43969662 | 43970696 | 181577, 237686 |
| 46597 | 44010104 | 44012212 | 237686 |
| 46598 | 44026031 | 44027992 | 237686 |
| 46599 | 44040368 | 44041328 | 237686 |
| 46600 | 44062173 | 44063250 | |
| 46601 | 44093858 | 44096792 | 137216, 180992 |
| 46602 | 44184963 | 44186206 | 262179 |
| 46603 | 44186708 | 44188130 | 112759 |
| 46604 | 44188924 | 44192419 | 112759 |
| 46605 | 44196195 | 44197724 | 112759 |
| 46606 | 44201671 | 44204385 | 112759 |
| 46607 | 44204391 | 44207687 | |
| 46608 | 44213148 | 44216585 | 96384 |
| 46609 | 44223774 | 44226151 | 146232, 157593 |
| 46610 | 44230543 | 44233706 | 146232 |
| 46611 | 44280345 | 44281561 | 124608, 272442 |
| 46612 | 44349398 | 44349857 | |
| 46613 | 44354082 | 44356195 | 96401 |
| 46614 | 44364629 | 44365195 | 96401 |
| 46615 | 44383833 | 44384304 | 96401 |
| 46616 | 44432302 | 44432761 | |
| 46617 | 44457144 | 44459483 | |
| 46618 | 44460734 | 44463474 | |
| 46619 | 44705745 | 44706922 | |
| 46620 | 44888540 | 44889939 | 196284 |
| 46621 | 44907990 | 44908715 | 196284 |
| 46622 | 44927794 | 44929529 | 196284 |
| 46623 | 44933446 | 44935589 | 196284 |
| 46624 | 45344281 | 45346884 | 196284, 124813 |
| 46625 | 45412889 | 45414443 | 124813 |
| 46626 | 45516345 | 45517179 | 124813 |
| 46627 | 45742471 | 45743416 | |
| 46628 | 45811264 | 45813815 | |
| 46629 | 45932907 | 45934177 | 112782 |
| 46630 | 46012853 | 46013495 | 112782 |
| 46631 | 46020927 | 46021728 | 112782 |
| 46632 | 46029793 | 46030726 | 112782 |
| 46633 | 46097046 | 46098509 | 001561, 231769 |
| 46634 | 46346572 | 46347265 | 172348 |
| 46635 | 46359900 | 46361598 | 172348 |
| 46636 | 46616503 | 46617706 | 146233 |
| 46637 | 46618680 | 46619299 | 146233 |
| 46638 | 46619810 | 46621687 | 153291, 146233 |
| 46639 | 46789063 | 46790045 | 112818 |
| 46640 | 46893305 | 46894286 | 69122 |
| 46641 | 47014889 | 47015776 | |
| 46642 | 47079316 | 47080150 | |
| 46643 | 47093829 | 47095335 | |
| 46644 | 47102253 | 47104028 | |
| 46645 | 47114690 | 47115599 | |
| 46646 | 47128809 | 47129309 | |
| 46647 | 47135383 | 47137142 | |
| 46648 | 47155214 | 47157194 | |
| 46649 | 47276257 | 47278286 | 146072 |
| 46650 | 47330250 | 47332109 | |
| 46651 | 47371181 | 47373499 | |
| 46652 | 47381544 | 47383286 | |
| 46653 | 47386700 | 47388952 | |
| 46654 | 47427495 | 47428649 | |
| 46655 | 47431751 | 47432428 | |
| 46656 | 47444329 | 47447152 | 198087, 270761 |
| 46657 | 47709377 | 47710144 | |
| 46658 | 48733853 | 48734731 | |
| 46659 | 49430035 | 49431322 | 031691, 146085 |
| 46660 | 49480992 | 49481811 | 203972 |
| 46661 | 49490498 | 49491486 | 203972 |
| 46662 | 49491555 | 49492081 | 203972 |
| 46663 | 49508196 | 49510164 | |
| 46664 | 49518011 | 49522134 | 197261 |
| 46665 | 49523284 | 49524780 | 197261 |
| 46666 | 49734253 | 49735123 | |
| 46667 | 50059322 | 50059818 | |
| 46668 | 50060275 | 50062296 | 226733 |
| 46669 | 51065895 | 51066785 | |
| 46670 | 51179737 | 51180229 | |
| 46671 | 51543623 | 51544713 | 170927 |
| 46672 | 51569682 | 51570410 | 170927 |
| 46673 | 51602264 | 51603045 | 170927 |
| 46674 | 51928276 | 51928989 | 170927 |
| 46675 | 51941768 | 51943386 | 170927 |
| 46676 | 51951857 | 51952894 | 170927 |
| 46677 | 51989276 | 51990531 | |
| 46678 | 52136454 | 52137162 | 112118 |
| 46679 | 52149113 | 52150388 | 112118 |
| 46680 | 52216985 | 52217815 | |
| 46681 | 52236375 | 52236774 | 170915 |
| 46682 | 52253883 | 52256009 | 170915 |
| 46683 | 52284263 | 52286516 | 96093 |
| 46684 | 52288541 | 52289654 | 96093 |
| 46685 | 52291312 | 52292141 | 96093 |
| 46686 | 52368497 | 52369481 | 065308, 096093 |
| 46687 | 52378696 | 52380340 | 065308, 096093 |
| 46688 | 52382237 | 52383860 | 065308, 096093 |
| 46689 | 52386385 | 52388356 | 096093, 065308 |
| 46690 | 52406846 | 52410764 | 65308 |
| 46691 | 52415444 | 52417593 | 65308 |
| 46692 | 52430721 | 52433190 | 65308 |
| 46693 | 52440521 | 52443052 | 225791, 065308 |
| 46694 | 52453277 | 52454499 | |
| 46695 | 52454714 | 52455774 | |
| 46696 | 52475558 | 52476233 | |
| 46697 | 52527865 | 52530177 | 216775 |
| 46698 | 52530264 | 52531281 | 216775 |
| 46699 | 52535134 | 52536786 | 96092 |
| 46700 | 52588425 | 52591982 | |
| 46701 | 52599501 | 52600512 | |
| 46702 | 52606609 | 52607869 | 237674 |
| 46703 | 52609862 | 52613095 | |
| 46704 | 52652178 | 52655871 | |
| 46705 | 52667682 | 52668242 | 243955 |
| 46706 | 52684862 | 52685597 | |
| 46707 | 52793941 | 52795236 | |
| 46708 | 52858847 | 52861058 | 202198, 170899 |
| 46709 | 52906801 | 52907588 | 112144 |
| 46710 | 52920702 | 52921670 | 112146, 112144 |
| 46711 | 52925487 | 52927488 | 112144, 112146 |
| 46712 | 52984290 | 52985274 | |
| 46713 | 53032832 | 53034375 | |
| 46714 | 53051376 | 53052185 | |
| 46715 | 53161784 | 53162418 | 12660 |
| 46716 | 53164032 | 53164599 | 12660 |
| 46717 | 53165455 | 53167754 | 12660 |
| 46718 | 53171286 | 53172755 | 12660 |
| 46719 | 53173861 | 53174378 | 12660 |
| 46720 | 53212365 | 53214802 | 12660 |
| 46721 | 53223162 | 53226009 | |
| 46722 | 53241243 | 53242934 | |
| 46723 | 53276056 | 53277192 | |
| 46724 | 53296286 | 53299222 | |
| 46725 | 53301652 | 53303256 | |
| 46726 | 53304137 | 53305675 | |
| 46727 | 53329192 | 53331417 | |
| 46728 | 53360631 | 53362067 | |
| 46729 | 53395026 | 53395598 | 1084 |
| 46730 | 53408237 | 53410266 | 1084 |
| 46731 | 53411892 | 53413882 | 1084 |
| 46732 | 53430655 | 53431145 | 231683, 001084 |
| 46733 | 53478027 | 53482850 | 231683, 001084 |
| 46734 | 53494638 | 53495172 | 235899 |
| 46735 | 53498016 | 53500708 | |
| 46736 | 53515207 | 53517694 | 124743 |
| 46737 | 53537340 | 53539609 | |
| 46738 | 53599262 | 53599810 | |
| 46739 | 53605681 | 53606205 | 227885 |
| 46740 | 53657765 | 53658904 | 228614 |
| 46741 | 53658930 | 53660335 | 137269, 228614 |
| 46742 | 53719264 | 53720358 | 137269 |
| 46743 | 53791402 | 53792087 | |
| 46744 | 53793753 | 53794631 | |
| 46745 | 53862922 | 53864857 | 236996, 146147 |
| 46746 | 53874782 | 53876968 | 146147 |
| 46747 | 53902190 | 53902962 | 146147, 235050 |
| 46748 | 53962533 | 53963011 | 146147 |
| 46749 | 53999733 | 54001391 | 146147 |
| 46750 | 54062803 | 54063425 | 146147 |
| 46751 | 54284180 | 54285570 | |
| 46752 | 54880940 | 54881656 | |
| 46753 | 56252189 | 56253664 | 124749 |
| 46754 | 56263776 | 56265900 | |
| 46755 | 56305332 | 56306129 | |
| 46756 | 56340692 | 56341193 | 151914 |
| 46757 | 56346217 | 56349781 | 151914 |
| 46758 | 56375630 | 56376237 | 151914 |
| 46759 | 56381927 | 56382672 | 151914 |
| 46760 | 56405774 | 56406616 | 151914 |
| 46761 | 56406892 | 56408074 | 151914 |
| 46762 | 56506725 | 56508946 | 151914 |
| 46763 | 56513328 | 56515992 | 151914 |
| 46764 | 56547240 | 56550317 | 151914 |
| 46765 | 56550513 | 56559868 | 151914 |
| 46766 | 56577358 | 56580859 | 151914 |
| 46767 | 56583711 | 56584663 | 151914 |
| 46768 | 56593240 | 56596568 | 151914 |
| 46769 | 56604996 | 56607504 | 151914 |
| 46770 | 56628565 | 56629497 | 151914 |
| 46771 | 56636114 | 56636875 | 151914 |
| 46772 | 56672137 | 56672552 | 151914 |
| 46773 | 56727803 | 56729292 | 231441, 151914 |
| 46774 | 56740302 | 56741527 | 151914 |
| 46775 | 56803223 | 56803808 | 151914 |
| 46776 | 56910997 | 56911691 | 168116 |
| 46777 | 56954511 | 56955153 | 112200 |
| 46778 | 57036455 | 57039388 | 112208, 226803 |
| 46779 | 57085879 | 57087683 | 112210 |
| 46780 | 57181289 | 57183085 | 146143 |
| 46781 | 57365048 | 57365871 | 146143 |
| 46782 | 58776533 | 58779338 | |
| 46783 | 64056947 | 64058540 | |
| 46784 | 64061848 | 64063988 | |
| 46785 | 64092727 | 64095869 | |
| 46786 | 64101305 | 64101929 | |
| 46787 | 64120437 | 64122005 | |
| 46788 | 64128548 | 64129073 | |
| 46789 | 64156976 | 64159255 | |
| 46790 | 64161453 | 64164600 | |
| 46791 | 64167442 | 64170601 | |
| 46792 | 64175003 | 64177604 | |
| 46793 | 64185138 | 64186848 | |
| 46794 | 64187293 | 64188726 | |
| 46795 | 64214474 | 64215559 | |
| 46796 | 64219197 | 64221129 | |
| 46797 | 64222474 | 64228107 | |
| 46798 | 64228282 | 64230089 | |
| 46799 | 64231631 | 64233665 | 112245 |
| 46800 | 64236827 | 64238764 | 112245 |
| 46801 | 64246847 | 64248154 | 112245 |
| 46802 | 64270607 | 64271863 | 112245 |
| 46803 | 64273747 | 64276721 | 112245 |
| 46804 | 64280230 | 64286491 | 266680, 112245 |
| 46805 | 64299709 | 64300533 | |
| 46806 | 64307574 | 64309026 | |
| 46807 | 64345420 | 64347524 | 118482 |
| 46808 | 64359248 | 64360580 | 118482 |
| 46809 | 64373335 | 64374158 | 118482 |
| 46810 | 64384408 | 64385584 | 118482 |
| 46811 | 64386801 | 64387928 | 118482 |
| 46812 | 64514141 | 64517517 | 236345, 188107 |
| 46813 | 64646062 | 64646732 | 188107 |
| 46814 | 70441909 | 70442269 | 168216 |
| 46815 | 70490200 | 70491598 | 168216 |
| 46816 | 70505687 | 70507307 | 168216 |
| 46817 | 70535510 | 70536268 | |
| 46818 | 70576472 | 70577553 | 82293 |
| 46819 | 71083749 | 71085901 | |
| 46820 | 71106651 | 71110076 | 237643 |
| 46821 | 71122297 | 71124439 | 082269, 224349 |
| 46822 | 71276420 | 71277506 | 154079 |
| 46823 | 71376912 | 71379049 | 112305 |
| 46824 | 71434023 | 71434657 | 112305 |
| 46825 | 71548277 | 71550067 | 112305 |
| 46826 | 71558829 | 71560165 | 112305 |
| 46827 | 71997811 | 71999467 | 119900, 232295 |
| 46828 | 72009539 | 72010741 | 119900, 269966, 232295 |
| 46829 | 72042570 | 72045185 | 269966 |
| 46830 | 72094907 | 72095634 | 269966, 233237 |
| 46831 | 72103069 | 72104353 | 269966, 233237 |
| 46832 | 72113310 | 72116805 | 207827, 269966, 233237 |
| 46833 | 72122934 | 72124703 | 269966, 233237 |
| 46834 | 72128960 | 72131298 | 233237 |
| 46835 | 72209284 | 72209842 | |
| 46836 | 72211505 | 72212305 | |
| 46837 | 73123472 | 73124371 | |
| 46838 | 74161096 | 74162300 | 164430 |
| 46839 | 74170714 | 74172846 | 135297 |
| 46840 | 74222760 | 74223738 | |
| 46841 | 74224190 | 74227316 | 156508 |
| 46842 | 74228731 | 74234437 | 229862, 156508 |
| 46843 | 74258291 | 74258834 | 229862 |
| 46844 | 74275804 | 74278369 | 229862 |
| 46845 | 74288709 | 74291327 | |
| 46846 | 74295031 | 74296733 | |
| 46847 | 74362770 | 74365482 | 119899 |
| 46848 | 74413843 | 74414445 | 156535 |
| 46849 | 74483975 | 74484825 | 156535 |
| 46850 | 74508164 | 74509566 | 156535 |
| 46851 | 74510716 | 74514629 | 156535 |
| 46852 | 74753766 | 74755054 | |
| 46853 | 75243375 | 75245320 | 272243, 223786 |
| 46854 | 75808402 | 75808869 | 111799 |
| 46855 | 75892723 | 75893296 | 111799 |
| 46856 | 75933637 | 75934612 | |
| 46857 | 75952607 | 75954412 | 112695 |
| 46858 | 75993331 | 75995697 | 225793, 112697 |
| 46859 | 76233952 | 76234647 | |
| 46860 | 76310576 | 76313229 | 112701 |
| 46861 | 76445260 | 76446392 | |
| 46862 | 76458030 | 76459907 | 196586 |
| 46863 | 76463110 | 76464212 | 196586 |
| 46864 | 77499506 | 77500805 | |
| 46865 | 77595056 | 77596104 | |
| 46866 | 77727212 | 77727758 | |
| 46867 | 77782485 | 77783447 | |
| 46868 | 78544201 | 78544827 | 269964 |
| 46869 | 79093665 | 79094164 | |
| 46870 | 79099535 | 79100860 | |
| 46871 | 79519887 | 79521580 | 229495 |
| 46872 | 79576749 | 79577875 | 146243 |
| 46873 | 79786275 | 79787284 | 146247 |
| 46874 | 79787376 | 79789289 | 146247 |
| 46875 | 79868429 | 79869260 | |
| 46876 | 79880555 | 79881272 | |
| 46877 | 79924882 | 79925340 | 118418 |
| 46878 | 79943110 | 79944616 | 270362, 118418 |
| 46879 | 80246544 | 80247833 | 135338 |
| 46880 | 80279824 | 80281074 | |
| 46881 | 80314198 | 80314984 | |
| 46882 | 80339958 | 80341861 | 198478 |
| 46883 | 80342308 | 80342839 | 198478 |
| 46884 | 80386465 | 80387446 | 198478 |
| 46885 | 80487323 | 80488495 | |
| 46886 | 80581885 | 80583087 | 233835 |
| 46887 | 80584308 | 80585040 | 233835 |
| 46888 | 80587741 | 80588541 | |
| 46889 | 80713449 | 80714706 | 112742 |
| 46890 | 80815679 | 80817157 | 83123 |
| 46891 | 80872766 | 80876065 | 83123 |
| 46892 | 80907179 | 80907815 | 83123 |
| 46893 | 80964323 | 80964864 | 83123 |
| 46894 | 81166791 | 81167437 | 233967 |
| 46895 | 81236995 | 81238357 | |
| 46896 | 81250608 | 81251749 | |
| 46897 | 81317427 | 81318108 | |
| 46898 | 81332366 | 81332858 | |
| 46899 | 81581592 | 81582428 | |
| 46900 | 81974503 | 81975772 | |
| 46901 | 82201083 | 82201898 | 112773 |
| 46902 | 82270719 | 82273267 | 112773 |
| 46903 | 82346386 | 82347381 | 112773 |
| 46904 | 82406135 | 82406683 | 112773 |
| 46905 | 82409878 | 82410664 | 112773 |
| 46906 | 82437412 | 82438226 | 112773, 232031 |
| 46907 | 82453219 | 82453836 | 112773 |
| 46908 | 82459924 | 82464351 | 112773 |
| 46909 | 82468875 | 82470353 | |
| 46910 | 82496342 | 82499464 | |
| 46911 | 82526010 | 82526685 | |
| 46912 | 82530195 | 82531522 | |
| 46913 | 82541004 | 82541407 | |
| 46914 | 82607339 | 82608410 | 226453 |
| 46915 | 82618567 | 82619694 | 226453 |
| 46916 | 82642760 | 82644554 | 226453 |
| 46917 | 82645936 | 82650255 | |
| 46918 | 82691141 | 82692067 | |
| 46919 | 82726977 | 82727465 | |
| 46920 | 82732062 | 82733019 | |
| 46921 | 82853273 | 82854570 | |
| 46922 | 82956132 | 82959119 | 5700 |
| 46923 | 83774778 | 83775857 | 118420 |
| 46924 | 83776994 | 83778588 | 83097 |
| 46925 | 83892260 | 83894385 | 13375 |
| 46926 | 83902223 | 83904316 | 013392, 013375 |
| 46927 | 83908766 | 83910131 | |
| 46928 | 84112888 | 84113241 | 65833 |
| 46929 | 84139851 | 84141502 | 65833 |
| 46930 | 84190083 | 84190588 | |
| 46931 | 84568848 | 84570158 | 65615 |
| 46932 | 84881681 | 84882754 | 135315 |
| 46933 | 84936756 | 84938134 | 135315 |
| 46934 | 84979334 | 84980798 | |
| 46935 | 85125850 | 85126359 | |
| 46936 | 85126931 | 85127390 | |
| 46937 | 85158910 | 85159537 | 231776 |
| 46938 | 85483717 | 85485042 | |
| 46939 | 85577098 | 85578303 | |
| 46940 | 86069922 | 86072203 | |
| 46941 | 86114410 | 86116032 | |
| 46942 | 86159143 | 86162461 | 135318 |
| 46943 | 86177408 | 86179798 | 135318 |
| 46944 | 86225745 | 86226355 | 135317 |
| 46945 | 86289103 | 86290976 | 271793, 135317 |
| 46946 | 86302644 | 86304652 | 135317, 271793 |
| 46947 | 86322616 | 86323338 | 271793, 135316 |
| 46948 | 86351194 | 86354727 | 135316 |
| 46949 | 86386946 | 86389412 | 203875 |
| 46950 | 86456997 | 86457971 | |
| 46951 | 87861148 | 87862550 | 188994, 272008 |
| 46952 | 87864228 | 87866335 | 272008, 188994 |
| 46953 | 88031982 | 88033087 | 226524, 111850, 164411 |
| 46954 | 88117473 | 88118638 | 213204, 272514 |
| 46955 | 88182022 | 88183827 | 213204, 164414 |
| 46956 | 88298786 | 88300675 | 135336, 146282 |
| 46957 | 88369735 | 88370326 | 135336 |
| 46958 | 88381045 | 88382287 | |
| 46959 | 88409863 | 88412649 | 135334 |
| 46960 | 88432052 | 88433778 | |
| 46961 | 88439909 | 88442868 | |
| 46962 | 88498681 | 88499763 | |
| 46963 | 88530108 | 88530520 | |
| 46964 | 89011924 | 89012642 | 234426 |
| 46965 | 89069556 | 89071003 | 234426 |
| 46966 | 89371650 | 89373044 | 111880 |
| 46967 | 89419466 | 89420161 | 111880 |
| 46968 | 89524444 | 89525743 | 111880 |
| 46969 | 89550499 | 89551135 | 111880 |
| 46970 | 89672406 | 89673782 | 111880 |
| 46971 | 89694966 | 89696265 | |
| 46972 | 89731487 | 89734368 | |
| 46973 | 89743057 | 89747725 | |
| 46974 | 89759823 | 89761095 | |
| 46975 | 89776934 | 89777996 | |
| 46976 | 89780490 | 89781280 | |
| 46977 | 89788733 | 89794286 | 146278, 271931 |
| 46978 | 89795893 | 89797685 | |
| 46979 | 89809687 | 89810469 | 154548 |
| 46980 | 89826795 | 89827886 | 154548 |
| 46981 | 89829980 | 89830446 | |
| 46982 | 89849978 | 89850644 | |
| 46983 | 89855275 | 89857028 | 146281 |
| 46984 | 90061291 | 90063294 | 198833 |
| 46985 | 90121233 | 90122796 | 25039 |
| 46986 | 90142510 | 90144198 | 135299 |
| 46987 | 90153238 | 90154263 | 135299 |
| 46988 | 90252527 | 90253097 | 135299 |
| 46989 | 90277176 | 90278092 | 083099, 135299 |
| 46990 | 90347399 | 90348943 | 83099 |
| 46991 | 90417847 | 90418786 | 112159 |
| 46992 | 90486412 | 90487060 | 112159 |
| 46993 | 90490655 | 90491474 | 112159 |
| 46994 | 90528532 | 90530329 | 112159 |
| 46995 | 90539146 | 90539982 | 118412 |
| 46996 | 90650065 | 90651604 | 112182 |
| 46997 | 90661341 | 90661928 | 226455, 112182 |
| 46998 | 90665930 | 90666446 | 112182 |
| 46999 | 90680273 | 90681272 | 112182 |
| 47000 | 90797726 | 90798256 | 112182 |
| 47001 | 90806727 | 90809104 | 112182 |
| 47002 | 90813120 | 90813589 | 112182 |
| 47003 | 90858773 | 90859450 | 112182 |
| 47004 | 90877241 | 90878942 | 112182 |
| 47005 | 90896374 | 90898034 | 112182 |
| 47006 | 90909610 | 90910062 | 112182 |
| 47007 | 90913230 | 90913885 | 112182 |
| 47008 | 90927261 | 90929797 | 112182 |
| 47009 | 90931576 | 90933273 | 112182 |
| 47010 | 90948180 | 90949983 | 112182 |
| 47011 | 90994073 | 90995801 | 112182 |
| 47012 | 90998114 | 90998707 | 112182 |
| 47013 | 91001185 | 91002099 | 112182 |
| 47014 | 91003113 | 91006948 | 112182 |
| 47015 | 91067431 | 91068535 | 260271 |
| 47016 | 91077642 | 91080060 | |
| 47017 | 91107295 | 91108193 | |
| 47018 | 91115648 | 91117158 | |
| 47019 | 91118458 | 91119442 | |
| 47020 | 91120785 | 91121465 | |
| 47021 | 91185448 | 91185918 | |
| 47022 | 91212313 | 91213344 | |
| 47023 | 91295949 | 91297331 | 135341 |
| 47024 | 92522291 | 92523055 | |
| 47025 | 93520533 | 93520967 | |
| 47026 | 94628658 | 94629593 | |
| 47027 | 96022594 | 96023366 | 261366 |
| 47028 | 96024803 | 96026413 | 172469, 261366 |
| 47029 | 96263240 | 96263758 | |
| 47030 | 96968817 | 96970880 | 014123, 233797 |
| 47031 | 97344856 | 97347409 | 123545 |
| 47032 | 97371536 | 97373072 | 186231 |
| 47033 | 98395713 | 98396289 | 271860 |
| 47034 | 99284502 | 99285357 | 184486 |
| 47035 | 99394727 | 99396252 | 112234 |
| 47036 | 99841188 | 99842493 | 132423 |
| 47037 | 99872424 | 99874323 | 228506, 132424 |
| 47038 | 99880703 | 99881123 | 123552 |
| 47039 | 99962545 | 99964490 | 123552 |
| 47040 | 99968608 | 99969996 | 123552, 228439 |
| 47041 | 100015220 | 100016976 | 272017, 112237 |
| 47042 | 100546844 | 100547762 | |
| 47043 | 100623706 | 100624319 | |
| 47044 | 100682338 | 100683115 | |
| 47045 | 100731094 | 100731731 | |
| 47046 | 100747759 | 100748798 | |
| 47047 | 100903066 | 100903924 | 112246 |
| 47048 | 100930482 | 100931041 | |
| 47049 | 100932161 | 100932894 | |
| 47050 | 100950371 | 100953448 | |
| 47051 | 101065848 | 101066494 | 112249 |
| 47052 | 101075450 | 101076095 | 112249 |
| 47053 | 101327992 | 101329970 | 260000, 112249 |
| 47054 | 101349207 | 101350243 | |
| 47055 | 101448264 | 101448693 | |
| 47056 | 102208915 | 102209696 | 164418 |
| 47057 | 102355194 | 102356029 | 164418 |
| 47058 | 103127666 | 103128179 | |
| 47059 | 103792483 | 103793197 | |
| 47060 | 104040723 | 104041165 | |
| 47061 | 105158747 | 105159745 | |
| 47062 | 105773909 | 105774453 | 85377 |
| 47063 | 105849544 | 105851762 | 85377 |
| 47064 | 105880577 | 105881206 | |
| 47065 | 106240939 | 106244595 | |
| 47066 | 106253429 | 106254914 | |
| 47067 | 106340659 | 106341133 | |
| 47068 | 106343981 | 106345003 | |
| 47069 | 106416415 | 106416898 | |
| 47070 | 106442524 | 106443466 | |
| 47071 | 106708936 | 106710261 | 57663 |
| 47072 | 106757754 | 106758326 | 57663 |
| 47073 | 106772423 | 106774516 | 57663 |
| 47074 | 106959450 | 106961323 | 112297 |
| 47075 | 106972631 | 106973895 | 112297 |
| 47076 | 106993614 | 106995031 | 112297 |
| 47077 | 107039212 | 107039728 | 130347 |
| 47078 | 107076496 | 107078589 | 130348, 130347 |
| 47079 | 107139850 | 107140903 | |
| 47080 | 107149702 | 107151716 | |
| 47081 | 107172594 | 107173391 | 235142 |
| 47082 | 107178535 | 107179895 | 235142 |
| 47083 | 107187056 | 107188843 | 235142 |
| 47084 | 107211672 | 107214179 | 235142 |
| 47085 | 107229895 | 107236466 | 207577, 235142 |
| 47086 | 107245821 | 107247907 | |
| 47087 | 107349091 | 107350323 | 130349 |
| 47088 | 107434997 | 107437528 | 178409 |
| 47089 | 107551129 | 107552357 | 220237, 164494 |
| 47090 | 107585441 | 107586063 | 164494 |
| 47091 | 107652700 | 107653453 | 164494 |
| 47092 | 107779645 | 107781896 | 164494 |
| 47093 | 107811092 | 107812126 | 112320 |
| 47094 | 107827297 | 107827663 | 112320 |
| 47095 | 107859599 | 107862852 | 112320 |
| 47096 | 107945370 | 107946301 | 112320 |
| 47097 | 107996088 | 107997007 | |
| 47098 | 108132746 | 108134219 | 146285 |
| 47099 | 108136959 | 108138782 | 146285 |
| 47100 | 108139238 | 108140385 | 146285 |
| 47101 | 108143440 | 108146035 | 146285 |
| 47102 | 108273763 | 108274356 | 25796 |
| 47103 | 108277961 | 108279910 | 272476, 025796 |
| 47104 | 108395043 | 108396849 | 81087 |
| 47105 | 108448937 | 108449742 | 225174, 081087 |
| 47106 | 108454598 | 108455742 | 225174, 081087 |
| 47107 | 108514376 | 108514728 | |
| 47108 | 108521828 | 108522476 | |
| 47109 | 108524332 | 108525344 | |
| 47110 | 108562514 | 108563094 | 112335 |
| 47111 | 108580969 | 108583541 | 112335 |
| 47112 | 108615152 | 108617407 | 135537 |
| 47113 | 108685202 | 108685717 | 135537 |
| 47114 | 108784007 | 108784669 | 135537 |
| 47115 | 108876493 | 108877104 | |
| 47116 | 108878390 | 108882319 | 118689 |
| 47117 | 108882697 | 108884752 | 118689 |
| 47118 | 108885208 | 108887875 | 118689 |
| 47119 | 108899683 | 108900282 | 118689 |
| 47120 | 108902324 | 108902870 | 118689 |
| 47121 | 108915714 | 108916262 | 118689 |
| 47122 | 108944326 | 108945844 | 118689 |
| 47123 | 108949192 | 108949795 | 118689 |
| 47124 | 109010171 | 109011193 | |
| 47125 | 109085037 | 109086246 | 203801 |
| 47126 | 109114418 | 109115199 | |
| 47127 | 109115949 | 109117004 | |
| 47128 | 109118915 | 109119882 | |
| 47129 | 109171427 | 109172622 | 118690 |
| 47130 | 109217161 | 109219591 | 118690 |
| 47131 | 109292141 | 109293342 | 118690 |
| 47132 | 109329108 | 109331716 | 80546 |
| 47133 | 109380331 | 109380753 | 199944, 080546 |
| 47134 | 109414695 | 109417425 | 183137, 080546 |
| 47135 | 109438255 | 109439263 | 183137 |
| 47136 | 109441934 | 109442987 | 183137 |
| 47137 | 109573331 | 109573978 | 243587 |
| 47138 | 109628825 | 109630379 | 203799 |
| 47139 | 109639265 | 109640146 | 203799 |
| 47140 | 109657404 | 109658541 | 203799 |
| 47141 | 109702355 | 109704885 | 260273, 135535 |
| 47142 | 109705402 | 109706040 | |
| 47143 | 109761420 | 109763116 | 135587, 185250 |
| 47144 | 109776192 | 109778955 | 135596 |
| 47145 | 109803728 | 109804846 | 112365 |
| 47146 | 110008253 | 110008930 | 155085 |
| 47147 | 110011820 | 110013008 | 112367, 155085 |
| 47148 | 110111346 | 110111901 | 112367 |
| 47149 | 110114129 | 110115338 | 112367 |
| 47150 | 110174800 | 110175618 | |
| 47151 | 110417931 | 110418490 | |
| 47152 | 110499689 | 110502514 | 168438, 112290 |
| 47153 | 110650470 | 110651765 | 53328 |
| 47154 | 110678623 | 110679911 | 53328 |
| 47155 | 110735809 | 110737276 | 203797 |
| 47156 | 110740297 | 110741151 | |
| 47157 | 110830752 | 110831599 | |
| 47158 | 110837960 | 110839283 | |
| 47159 | 110858201 | 110858914 | |
| 47160 | 110876144 | 110876782 | |
| 47161 | 110885595 | 110888605 | |
| 47162 | 111002333 | 111004128 | 155111 |
| 47163 | 111025053 | 111025676 | 155111 |
| 47164 | 111059925 | 111060794 | 155111 |
| 47165 | 111135563 | 111137270 | 155111 |
| 47166 | 111143262 | 111144121 | |
| 47167 | 111147444 | 111148586 | |
| 47168 | 111194995 | 111198807 | 123505 |
| 47169 | 111272907 | 111274693 | |
| 47170 | 111279015 | 111281071 | 155115 |
| 47171 | 111302484 | 111304316 | 197498 |
| 47172 | 111351115 | 111352155 | |
| 47173 | 111408169 | 111409245 | 112394 |
| 47174 | 111426578 | 111427262 | 112394 |
| 47175 | 111497328 | 111498312 | 112394 |
| 47176 | 111508867 | 111509447 | 112394 |
| 47177 | 111551521 | 111552175 | 231046, 112394 |
| 47178 | 111580006 | 111581315 | 173214, 231046 |
| 47179 | 111599924 | 111600342 | 230177 |
| 47180 | 111628467 | 111628960 | 9413 |
| 47181 | 111677724 | 111678142 | 9413 |
| 47182 | 111784169 | 111785470 | 9413 |
| 47183 | 111802518 | 111806617 | 231889, 009413 |
| 47184 | 111872846 | 111873989 | 231889 |
| 47185 | 111875904 | 111876875 | 231889 |
| 47186 | 111880208 | 111881328 | 231889, 056972 |
| 47187 | 111909949 | 111913406 | 231889, 056972 |
| 47188 | 111918776 | 111919786 | 231889, 056972 |
| 47189 | 111923143 | 111924429 | 255389, 056972 |
| 47190 | 111925454 | 111927760 | 56972 |
| 47191 | 111931596 | 111932509 | |
| 47192 | 111963501 | 111964245 | |
| 47193 | 111978081 | 111979393 | |
| 47194 | 112004267 | 112006149 | 10810 |
| 47195 | 112007408 | 112008322 | 10810 |
| 47196 | 112013094 | 112014674 | 10810 |
| 47197 | 112339333 | 112340557 | |
| 47198 | 112351916 | 112352715 | |
| 47199 | 112364039 | 112365363 | |
| 47200 | 112407731 | 112409283 | 203778, 074935 |
| 47201 | 112519812 | 112520383 | 112769 |
| 47202 | 112523848 | 112527525 | 112769 |
| 47203 | 112535872 | 112536864 | 112769 |
| 47204 | 112539627 | 112540428 | 112769, 270661 |
| 47205 | 112543052 | 112544109 | 112769 |
| 47206 | 112574663 | 112576088 | 226440, 112769 |
| 47207 | 112652168 | 112653093 | 226440 |
| 47208 | 113672295 | 113675007 | |
| 47209 | 113754631 | 113755262 | |
| 47210 | 113950758 | 113954871 | 230943 |
| 47211 | 113955002 | 113957202 | 230943 |
| 47212 | 113957320 | 113958339 | 230943 |
| 47213 | 113963294 | 113965060 | 230943 |
| 47214 | 113970300 | 113971203 | 230943 |
| 47215 | 113974640 | 113976477 | |
| 47216 | 114011813 | 114012285 | |
| 47217 | 114039748 | 114041840 | |
| 47218 | 114084020 | 114085616 | |
| 47219 | 114175868 | 114176525 | |
| 47220 | 114177097 | 114182026 | 155130 |
| 47221 | 114193851 | 114195201 | 227502 |
| 47222 | 114291206 | 114293202 | 196591, 228624 |
| 47223 | 115934420 | 115935155 | |
| 47224 | 116088079 | 116088646 | |
| 47225 | 116104727 | 116105205 | |
| 47226 | 116142908 | 116143472 | |
| 47227 | 116151514 | 116152817 | |
| 47228 | 116268842 | 116269659 | 111816 |
| 47229 | 116290167 | 116291721 | 111816 |
| 47230 | 116331744 | 116335233 | 111816 |
| 47231 | 116355420 | 116356488 | 111816 |
| 47232 | 116356665 | 116358473 | 111816 |
| 47233 | 116362074 | 116362558 | 111816 |
| 47234 | 116380605 | 116383124 | 111816 |
| 47235 | 116393506 | 116394065 | |
| 47236 | 116408479 | 116408948 | |
| 47237 | 116421291 | 116422774 | 178425 |
| 47238 | 116531283 | 116532690 | 178425 |
| 47239 | 116574153 | 116577112 | 111817, 237021, 187189 |
| 47240 | 116599798 | 116601636 | 240050, 189241, 111817 |
| 47241 | 116691882 | 116693039 | 111817 |
| 47242 | 116891540 | 116893776 | 111832 |
| 47243 | 116988859 | 116990129 | 153975 |
| 47244 | 117269568 | 117270167 | |
| 47245 | 117275945 | 117276446 | |
| 47246 | 117708507 | 117709038 | 047932, 047936 |
| 47247 | 117710440 | 117711551 | 047932, 047936 |
| 47248 | 117712459 | 117713830 | 047932, 047936 |
| 47249 | 117761518 | 117763525 | 47932 |
| 47250 | 117802930 | 117805576 | 047932, 164465 |
| 47251 | 117836197 | 117837108 | 047932, 164465 |
| 47252 | 117865323 | 117866982 | 047932, 164465 |
| 47253 | 117922836 | 117924546 | 47932 |
| 47254 | 117995799 | 117996845 | 153989 |
| 47255 | 118894862 | 118896193 | 111860 |
| 47256 | 118971241 | 118973175 | 111860 |
| 47257 | 119029996 | 119030925 | 111860 |
| 47258 | 119213937 | 119218102 | 111875, 111877 |
| 47259 | 119254737 | 119256679 | 253194, 111877 |
| 47260 | 119356005 | 119356616 | 111879 |
| 47261 | 119392604 | 119393392 | 111879 |
| 47262 | 119398647 | 119400790 | 111879 |
| 47263 | 119450171 | 119452011 | 111879 |
| 47264 | 119467753 | 119470565 | 111879 |
| 47265 | 119491450 | 119492927 | |
| 47266 | 119493780 | 119494443 | |
| 47267 | 119530430 | 119531193 | 111885 |
| 47268 | 119558414 | 119558870 | 111885 |
| 47269 | 119580190 | 119580636 | 111885 |
| 47270 | 119582333 | 119582983 | 111885 |
| 47271 | 119628342 | 119629823 | 111885 |
| 47272 | 119667170 | 119672597 | 111885 |
| 47273 | 119908612 | 119909118 | |
| 47274 | 119949294 | 119949961 | |
| 47275 | 120532723 | 120533909 | |
| 47276 | 121313368 | 121314024 | |
| 47277 | 121314982 | 121316152 | |
| 47278 | 121655248 | 121656811 | 146350 |
| 47279 | 121751460 | 121751991 | |
| 47280 | 122720257 | 122721684 | 25156 |
| 47281 | 122791770 | 122793243 | 135549, 111897 |
| 47282 | 122930927 | 122932400 | 135549 |
| 47283 | 123092027 | 123092762 | |
| 47284 | 123109660 | 123113871 | 172594 |
| 47285 | 123118519 | 123120748 | 172594 |
| 47286 | 123376771 | 123379441 | 146352 |
| 47287 | 123411013 | 123414322 | |
| 47288 | 124277529 | 124278422 | 188580 |
| 47289 | 124283419 | 124284036 | 188580 |
| 47290 | 124692731 | 124693311 | 188580 |
| 47291 | 125282200 | 125286304 | 146373, 236548 |
| 47292 | 125295802 | 125296603 | 146373 |
| 47293 | 125298058 | 125298774 | 146373 |
| 47294 | 125316882 | 125317441 | 146373 |
| 47295 | 125378923 | 125379549 | 146373 |
| 47296 | 125420414 | 125421361 | |
| 47297 | 125443707 | 125446003 | 111907 |
| 47298 | 125474574 | 125476422 | 111907 |
| 47299 | 125490149 | 125490627 | 111907 |
| 47300 | 125495729 | 125496234 | 111907 |
| 47301 | 125518682 | 125519773 | 111907 |
| 47302 | 125522296 | 125525111 | 111907 |
| 47303 | 125621872 | 125623597 | 111906 |
| 47304 | 125628375 | 125629052 | |
| 47305 | 125632977 | 125634299 | |
| 47306 | 125832781 | 125833587 | |
| 47307 | 125906513 | 125907708 | 237742 |
| 47308 | 126067431 | 126071603 | 135547, 237742 |
| 47309 | 126101107 | 126103218 | 111912 |
| 47310 | 126110978 | 126113204 | 111912 |
| 47311 | 126220248 | 126222433 | 111912 |
| 47312 | 126227651 | 126228212 | 111912 |
| 47313 | 126239044 | 126241326 | 111912 |
| 47314 | 126252082 | 126252750 | 111912 |
| 47315 | 126263785 | 126264645 | |
| 47316 | 126264965 | 126268062 | |
| 47317 | 126277610 | 126279582 | 111911 |
| 47318 | 126306616 | 126308976 | 66651 |
| 47319 | 126309921 | 126311992 | 66651 |
| 47320 | 126316206 | 126316744 | 66651 |
| 47321 | 126361193 | 126363049 | |
| 47322 | 126375331 | 126376107 | |
| 47323 | 126501677 | 126505144 | 231842 |
| 47324 | 126658309 | 126658924 | |
| 47325 | 126700889 | 126702102 | |
| 47326 | 126858520 | 126860864 | |
| 47327 | 127422615 | 127424012 | |
| 47328 | 127536600 | 127537697 | |
| 47329 | 127587039 | 127589792 | 118518 |
| 47330 | 127663232 | 127665544 | 93144 |
| 47331 | 127686986 | 127688443 | |
| 47332 | 127741549 | 127742067 | |
| 47333 | 127779116 | 127780224 | 214338, 189367, 255330 |
| 47334 | 127802176 | 127803970 | 214338, 255330 |
| 47335 | 127804158 | 127806802 | 214338, 255330 |
| 47336 | 127843785 | 127844303 | 184530 |
| 47337 | 128326101 | 128326674 | 152894 |
| 47338 | 128383719 | 128386140 | 152894, 227945 |
| 47339 | 128445768 | 128446325 | 152894 |
| 47340 | 128529868 | 128530470 | 152894 |
| 47341 | 128660613 | 128661992 | 152894 |
| 47342 | 128679318 | 128680578 | 152894 |
| 47343 | 128686676 | 128687206 | 152894 |
| 47344 | 128785099 | 128785633 | 152894 |
| 47345 | 128799455 | 128800304 | 152894 |
| 47346 | 128816903 | 128817566 | 152894 |
| 47347 | 128819768 | 128822487 | 224733, 152894 |
| 47348 | 128825043 | 128827625 | 227112, 152894 |
| 47349 | 128828090 | 128834319 | 152894 |
| 47350 | 128839424 | 128842903 | 152894 |
| 47351 | 128976410 | 128978638 | |
| 47352 | 129814488 | 129815332 | 196569 |
| 47353 | 129896352 | 129896849 | |
| 47354 | 129962343 | 129962934 | 146376 |
| 47355 | 129992888 | 129995861 | 146376 |
| 47356 | 130068943 | 130070648 | 211996 |
| 47357 | 130116149 | 130116927 | |
| 47358 | 130341108 | 130341941 | 198945 |
| 47359 | 130341986 | 130342622 | 198945 |
| 47360 | 130364453 | 130365028 | 198945 |
| 47361 | 130499816 | 130500235 | 164483 |
| 47362 | 130628937 | 130629535 | 164483 |
| 47363 | 131136426 | 131137298 | |
| 47364 | 131147851 | 131150465 | 256162 |
| 47365 | 131171912 | 131172510 | 79819 |
| 47366 | 131174288 | 131176042 | 79819 |
| 47367 | 131184299 | 131185339 | 79819 |
| 47368 | 131359909 | 131360718 | 79819 |
| 47369 | 131383311 | 131385206 | 79819 |
| 47370 | 131422637 | 131424945 | |
| 47371 | 131428177 | 131428821 | |
| 47372 | 131450816 | 131451622 | |
| 47373 | 131455646 | 131457738 | 118507 |
| 47374 | 131537062 | 131538211 | 118507 |
| 47375 | 131602387 | 131604283 | 118507 |
| 47376 | 131682683 | 131684226 | |
| 47377 | 131688114 | 131690213 | |
| 47378 | 131715241 | 131717776 | |
| 47379 | 131868889 | 131870305 | |
| 47380 | 131872915 | 131874210 | |
| 47381 | 131894931 | 131897415 | 112282, 118520 |
| 47382 | 131948448 | 131949829 | 154269, 112282 |
| 47383 | 131972825 | 131973312 | 154269 |
| 47384 | 132087974 | 132088672 | |
| 47385 | 132128565 | 132129907 | 197594 |
| 47386 | 132140228 | 132141352 | 219532, 197594 |
| 47387 | 132142965 | 132143928 | 197594 |
| 47388 | 132146930 | 132147592 | 237115, 197594 |
| 47389 | 132157388 | 132158145 | 197594 |
| 47390 | 132165181 | 132166616 | 197594 |
| 47391 | 132166845 | 132168138 | 197594 |
| 47392 | 132169738 | 132171370 | 197594 |
| 47393 | 132215586 | 132216789 | 197594 |
| 47394 | 132258673 | 132260377 | |
| 47395 | 132270594 | 132273459 | 227220, 118523 |
| 47396 | 132279435 | 132281140 | 227220 |
| 47397 | 132301741 | 132302828 | 227220 |
| 47398 | 132436708 | 132437436 | |
| 47399 | 132654236 | 132656442 | 79931 |
| 47400 | 132833442 | 132835384 | 79950 |
| 47401 | 132916903 | 132918201 | |
| 47402 | 132919550 | 132920133 | |
| 47403 | 132928587 | 132929359 | |
| 47404 | 132933720 | 132934906 | |
| 47405 | 133007584 | 133009173 | 112299 |
| 47406 | 133014418 | 133015333 | 112299 |
| 47407 | 133032873 | 133036001 | 112299 |
| 47408 | 133036944 | 133037876 | |
| 47409 | 133040239 | 133041575 | |
| 47410 | 133050373 | 133053049 | 93134 |
| 47411 | 133054655 | 133058580 | 93134 |
| 47412 | 133062329 | 133063080 | |
| 47413 | 133071962 | 133072419 | 112303 |
| 47414 | 133084804 | 133086155 | 271488 |
| 47415 | 133118824 | 133120684 | 146409 |
| 47416 | 133133412 | 133137109 | 206754, 112306 |
| 47417 | 133258511 | 133259151 | |
| 47418 | 133284004 | 133286046 | |
| 47419 | 133755199 | 133757198 | 223542, 112319 |
| 47420 | 134126909 | 134127945 | 227954 |
| 47421 | 134135831 | 134136376 | 227954 |
| 47422 | 134260646 | 134261717 | |
| 47423 | 134268451 | 134269764 | |
| 47424 | 134272869 | 134275183 | 28839 |
| 47425 | 134299990 | 134301154 | 28839 |
| 47426 | 134312144 | 134312738 | 146411 |
| 47427 | 134347639 | 134348337 | 146411 |
| 47428 | 134370197 | 134370719 | 146411 |
| 47429 | 134373001 | 134374038 | 146411 |
| 47430 | 134378332 | 134379254 | |
| 47431 | 134379725 | 134382069 | |
| 47432 | 134482151 | 134482711 | |
| 47433 | 134494775 | 134498031 | 118515 |
| 47434 | 134498828 | 134500253 | 118515 |
| 47435 | 134520916 | 134521579 | 118515 |
| 47436 | 134567925 | 134569002 | 118515 |
| 47437 | 134570910 | 134571623 | 118515 |
| 47438 | 134616865 | 134617663 | 118515 |
| 47439 | 134693779 | 134694902 | |
| 47440 | 134816754 | 134817512 | 236700, 231971 |
| 47441 | 134823567 | 134825215 | 236700, 231971 |
| 47442 | 134838214 | 134838985 | 231971 |
| 47443 | 134850915 | 134851592 | 232310 |
| 47444 | 134872431 | 134873098 | 232310 |
| 47445 | 134951494 | 134952434 | 232310 |
| 47446 | 134979179 | 134980180 | 232310 |
| 47447 | 134992624 | 134993646 | |
| 47448 | 135108150 | 135109053 | |
| 47449 | 135143108 | 135144683 | |
| 47450 | 135147966 | 135148823 | |
| 47451 | 135152027 | 135152554 | |
| 47452 | 135161259 | 135162141 | |
| 47453 | 135170946 | 135171634 | |
| 47454 | 135184168 | 135186194 | |
| 47455 | 135229123 | 135231457 | |
| 47456 | 135254825 | 135255960 | 118514 |
| 47457 | 135269630 | 135271526 | 118514 |
| 47458 | 135304907 | 135305865 | 112339 |
| 47459 | 135375161 | 135376877 | 232876, 112339 |
| 47460 | 135409696 | 135410684 | 112339 |
| 47461 | 135443425 | 135444066 | |
| 47462 | 135466038 | 135467167 | |
| 47463 | 135572543 | 135573439 | |
| 47464 | 135818121 | 135820272 | 231028, 135541 |
| 47465 | 135844170 | 135844946 | 231028 |
| 47466 | 135948250 | 135948989 | 231028 |
| 47467 | 136002179 | 136003091 | 231028 |
| 47468 | 136043336 | 136044824 | |
| 47469 | 136134891 | 136136035 | |
| 47470 | 136151116 | 136152042 | |
| 47471 | 136172555 | 136173908 | 171408 |
| 47472 | 136225708 | 136226355 | 171408 |
| 47473 | 136230768 | 136231493 | 171408 |
| 47474 | 136243094 | 136244135 | 171408 |
| 47475 | 136256498 | 136258795 | 171408 |
| 47476 | 136388369 | 136389569 | 171408, 227844 |
| 47477 | 136479457 | 136480058 | 171408, 237596 |
| 47478 | 136494893 | 136495924 | 171408, 237596 |
| 47479 | 136546628 | 136548773 | 237596 |
| 47480 | 136571028 | 136571992 | 146410 |
| 47481 | 136609346 | 136612411 | 29363 |
| 47482 | 136810342 | 136811192 | 135525 |
| 47483 | 136828579 | 136831129 | 135525 |
| 47484 | 136831222 | 136834063 | 135525 |
| 47485 | 136845388 | 136847039 | 135525 |
| 47486 | 136870561 | 136872381 | 272189, 135525 |
| 47487 | 136928078 | 136928990 | 197442 |
| 47488 | 136948078 | 136948944 | 197442 |
| 47489 | 136964864 | 136965956 | 234263, 197442 |
| 47490 | 136990263 | 136991112 | 197442 |
| 47491 | 137058736 | 137060237 | 197442 |
| 47492 | 137095286 | 137096670 | 197442 |
| 47493 | 137112453 | 137114973 | 197442 |
| 47494 | 137143234 | 137144500 | 112357 |
| 47495 | 137437964 | 137438346 | |
| 47496 | 137440082 | 137441299 | |
| 47497 | 137443038 | 137444942 | |
| 47498 | 137466539 | 137467065 | 164485 |
| 47499 | 137469211 | 137470469 | 164485 |
| 47500 | 137537181 | 137541427 | 27697 |
| 47501 | 137554907 | 137555780 | |
| 47502 | 137557828 | 137559738 | |
| 47503 | 137562058 | 137563544 | |
| 47504 | 137568565 | 137569585 | |
| 47505 | 137582226 | 137583060 | |
| 47506 | 137630264 | 137631033 | |
| 47507 | 137673009 | 137675582 | |
| 47508 | 137736226 | 137737159 | |
| 47509 | 137980968 | 137982093 | |
| 47510 | 138001921 | 138003269 | |
| 47511 | 138010196 | 138011146 | |
| 47512 | 138018470 | 138019239 | |
| 47513 | 138028528 | 138029552 | |
| 47514 | 138030268 | 138032150 | |
| 47515 | 138043121 | 138046624 | |
| 47516 | 138049506 | 138050765 | |
| 47517 | 138053082 | 138053773 | 234956 |
| 47518 | 138055952 | 138056291 | 234956 |
| 47519 | 138059165 | 138060188 | 234956 |
| 47520 | 138128553 | 138130888 | |
| 47521 | 138131481 | 138133744 | |
| 47522 | 138133793 | 138135243 | |
| 47523 | 138186139 | 138190234 | 118503, 237499 |
| 47524 | 138192987 | 138197136 | 118503 |
| 47525 | 138222286 | 138223181 | |
| 47526 | 138225586 | 138226805 | |
| 47527 | 138407564 | 138410332 | 112378 |
| 47528 | 138426293 | 138429576 | 112378 |
| 47529 | 138482409 | 138483522 | 112379 |
| 47530 | 138618951 | 138619509 | 112379 |
| 47531 | 138692203 | 138693358 | |
| 47532 | 138708603 | 138709198 | |
| 47533 | 138724208 | 138726741 | 051620, 215878 |
| 47534 | 138732389 | 138732934 | 51620 |
| 47535 | 138742499 | 138743322 | 135540, 051620 |
| 47536 | 138760592 | 138761123 | 135540 |
| 47537 | 138818724 | 138821058 | 135540 |
| 47538 | 138821183 | 138821692 | 135540 |
| 47539 | 138824322 | 138826492 | 135540 |
| 47540 | 138828455 | 138829928 | 135540 |
| 47541 | 138862545 | 138863031 | 135540 |
| 47542 | 138864655 | 138865210 | 135540 |
| 47543 | 138870867 | 138872132 | 135540 |
| 47544 | 138876516 | 138877195 | 135540 |
| 47545 | 138881317 | 138882023 | 135540 |
| 47546 | 138913724 | 138915860 | 135540 |
| 47547 | 138918292 | 138919423 | 135540 |
| 47548 | 138964150 | 138964858 | 135540 |
| 47549 | 138982562 | 138984519 | 135540 |
| 47550 | 139012179 | 139015188 | 225177, 135540 |
| 47551 | 139045081 | 139045751 | |
| 47552 | 139094209 | 139095466 | 24862 |
| 47553 | 139278500 | 139279087 | 135597 |
| 47554 | 139308067 | 139310769 | 135597 |
| 47555 | 139315554 | 139316346 | |
| 47556 | 139317198 | 139318392 | |
| 47557 | 139336038 | 139336757 | |
| 47558 | 139348191 | 139351616 | 146386 |
| 47559 | 139456628 | 139457547 | 112406 |
| 47560 | 139629908 | 139630760 | |
| 47561 | 139657592 | 139658259 | |
| 47562 | 139690095 | 139693067 | |
| 47563 | 139693186 | 139698674 | 164442 |
| 47564 | 139699079 | 139700879 | |
| 47565 | 139709454 | 139709978 | |
| 47566 | 139801337 | 139802040 | |
| 47567 | 139806792 | 139807717 | |
| 47568 | 139811414 | 139812262 | |
| 47569 | 139908522 | 139909752 | |
| 47570 | 139918061 | 139920031 | |
| 47571 | 139947634 | 139948198 | |
| 47572 | 139975108 | 139977669 | |
| 47573 | 139997734 | 139998511 | |
| 47574 | 140000423 | 140001216 | |
| 47575 | 140007285 | 140008074 | |
| 47576 | 140019676 | 140021520 | |
| 47577 | 140029663 | 140030420 | |
| 47578 | 140051410 | 140052063 | |
| 47579 | 140063647 | 140065912 | |
| 47580 | 140069646 | 140070425 | |
| 47581 | 140074421 | 140075573 | |
| 47582 | 140075730 | 140077035 | |
| 47583 | 140083641 | 140084653 | |
| 47584 | 140084866 | 140087703 | |
| 47585 | 140154586 | 140155247 | |
| 47586 | 140174351 | 140175250 | |
| 47587 | 140204054 | 140205056 | |
| 47588 | 140224798 | 140226140 | |
| 47589 | 140231017 | 140231741 | |
| 47590 | 140263853 | 140265553 | |
| 47591 | 140276476 | 140277486 | |
| 47592 | 140298866 | 140299512 | |
| 47593 | 140332536 | 140333445 | |
| 47594 | 140364481 | 140366101 | |
| 47595 | 140370519 | 140374964 | |
| 47596 | 140392267 | 140393375 | 236013 |
| 47597 | 140426755 | 140427499 | |
| 47598 | 140639259 | 140640967 | |
| 47599 | 140663644 | 140666464 | |
| 47600 | 140701061 | 140702464 | |
| 47601 | 140766419 | 140767727 | |
| 47602 | 140781352 | 140782172 | |
| 47603 | 140783083 | 140787069 | |
| 47604 | 140857499 | 140860736 | |
| 47605 | 141046613 | 141047281 | |
| 47606 | 141242472 | 141243194 | |
| 47607 | 141347381 | 141348233 | |
| 47608 | 141586495 | 141587904 | |
| 47609 | 141647499 | 141648757 | |
| 47610 | 141701820 | 141703227 | |
| 47611 | 141870089 | 141870627 | |
| 47612 | 141873011 | 141874873 | |
| 47613 | 141882947 | 141884963 | |
| 47614 | 142195957 | 142196633 | |
| 47615 | 142198394 | 142199041 | |
| 47616 | 142277292 | 142278482 | |
| 47617 | 142289670 | 142290487 | |
| 47618 | 142467851 | 142469174 | 9844 |
| 47619 | 142579803 | 142581380 | 237494 |
| 47620 | 142600160 | 142601637 | |
| 47621 | 142606193 | 142607267 | |
| 47622 | 142618294 | 142620060 | |
| 47623 | 142621036 | 142625655 | 112414 |
| 47624 | 142656162 | 142657186 | 112414 |
| 47625 | 142693486 | 142698086 | 112414 |
| 47626 | 142726953 | 142727895 | 112414 |
| 47627 | 142733828 | 142735725 | 112414 |
| 47628 | 142891032 | 142892032 | 236366 |
| 47629 | 142955543 | 142957035 | 236366 |
| 47630 | 143016217 | 143018654 | |
| 47631 | 143122561 | 143124500 | 10818 |
| 47632 | 143140567 | 143144415 | 10818 |
| 47633 | 143158655 | 143161633 | 10818 |
| 47634 | 143165862 | 143169597 | 10818 |
| 47635 | 143170919 | 143173782 | 10818 |
| 47636 | 143174836 | 143175464 | 10818 |
| 47637 | 143192493 | 143194425 | 10818 |
| 47638 | 143205873 | 143207042 | 10818 |
| 47639 | 143223668 | 143224178 | 10818 |
| 47640 | 143246601 | 143249415 | 10818 |
| 47641 | 143264708 | 143269708 | 224460, 010818 |
| 47642 | 143380234 | 143383697 | 146416 |
| 47643 | 143428212 | 143429455 | 146416 |
| 47644 | 143481130 | 143482345 | 146416 |
| 47645 | 143544599 | 143546246 | 146416 |
| 47646 | 143588881 | 143589408 | 146416 |
| 47647 | 143717775 | 143719608 | |
| 47648 | 143729386 | 143730924 | |
| 47649 | 143770832 | 143773117 | 034693, 223203, 189007 |
| 47650 | 143831959 | 143833522 | 1036 |
| 47651 | 143856243 | 143856762 | |
| 47652 | 143857671 | 143859138 | 112419 |
| 47653 | 143903166 | 143903672 | 112419 |
| 47654 | 143997072 | 143997562 | 112419 |
| 47655 | 143998672 | 144000380 | 112419 |
| 47656 | 144066280 | 144067137 | 112419 |
| 47657 | 144163639 | 144165700 | 135521, 257065 |
| 47658 | 144384702 | 144386272 | 118495 |
| 47659 | 144415605 | 144417657 | 169976 |
| 47660 | 144470982 | 144473010 | 135604 |
| 47661 | 144479315 | 144479777 | 135604 |
| 47662 | 144536532 | 144538180 | |
| 47663 | 144584849 | 144587879 | |
| 47664 | 144605083 | 144608702 | 152818 |
| 47665 | 144618867 | 144619869 | 152818 |
| 47666 | 144670684 | 144671338 | 152818 |
| 47667 | 144696320 | 144697138 | 152818 |
| 47668 | 144736144 | 144738096 | 152818 |
| 47669 | 144869585 | 144870262 | 152818 |
| 47670 | 145241386 | 145243280 | |
| 47671 | 145336615 | 145337856 | |
| 47672 | 145340780 | 145342541 | |
| 47673 | 145346205 | 145348927 | |
| 47674 | 145355934 | 145356363 | |
| 47675 | 145372678 | 145373347 | |
| 47676 | 145469309 | 145469926 | |
| 47677 | 145588019 | 145588845 | |
| 47678 | 145693531 | 145694554 | |
| 47679 | 145803586 | 145805732 | |
| 47680 | 146055387 | 146057642 | 270638, 112425 |
| 47681 | 146070656 | 146071284 | |
| 47682 | 146134376 | 146137516 | 235652, 118496 |
| 47683 | 146224675 | 146225303 | 146414 |
| 47684 | 146269431 | 146269945 | 146414 |
| 47685 | 146284456 | 146285991 | 146414 |
| 47686 | 146864009 | 146868598 | 118508 |
| 47687 | 147178465 | 147179289 | 233452 |
| 47688 | 147181647 | 147182364 | 227748, 233452 |
| 47689 | 147189769 | 147190365 | 233452 |
| 47690 | 147271283 | 147272143 | 272397, 233452 |
| 47691 | 147523617 | 147526230 | 164506, 233452 |
| 47692 | 147827467 | 147832674 | 203727 |
| 47693 | 147851149 | 147852916 | 203727 |
| 47694 | 148295349 | 148295956 | |
| 47695 | 148449205 | 148449658 | |
| 47696 | 148512854 | 148513502 | |
| 47697 | 148663207 | 148664829 | 111961 |
| 47698 | 148762037 | 148763330 | 111961 |
| 47699 | 149352793 | 149355036 | 236591, 111962 |
| 47700 | 149451307 | 149452114 | |
| 47701 | 149553660 | 149554980 | 055208, 228408 |
| 47702 | 149637618 | 149641357 | 55208 |
| 47703 | 149655361 | 149659149 | 55208 |
| 47704 | 149775356 | 149776098 | 178199 |
| 47705 | 149806135 | 149806901 | 178199 |
| 47706 | 149866219 | 149867620 | 131013 |
| 47707 | 149886612 | 149888497 | 55211 |
| 47708 | 149968907 | 149970205 | 186625 |
| 47709 | 150037741 | 150040113 | 131023 |
| 47710 | 150067205 | 150068034 | 120253 |
| 47711 | 150070575 | 150071616 | 120265, 120253 |
| 47712 | 150073740 | 150074198 | 120265 |
| 47713 | 150160904 | 150162335 | 120256 |
| 47714 | 150168050 | 150169152 | 120256 |
| 47715 | 150183672 | 150186562 | 268592, 120256 |
| 47716 | 150189096 | 150190152 | 268592 |
| 47717 | 150206155 | 150209820 | 223701, 164520, 268592 |
| 47718 | 150216420 | 150219382 | 164520, 223701, 268592 |
| 47719 | 150325837 | 150326754 | 218358 |
| 47720 | 150346392 | 150347081 | 155918 |
| 47721 | 150389606 | 150390654 | 131019 |
| 47722 | 150689710 | 150691039 | 9765 |
| 47723 | 150698383 | 150699168 | 9765 |
| 47724 | 150712338 | 150715430 | 9765 |
| 47725 | 150724763 | 150725445 | 9765 |
| 47726 | 150730463 | 150732986 | |
| 47727 | 150733140 | 150733747 | |
| 47728 | 150738558 | 150741024 | |
| 47729 | 150778080 | 150779048 | |
| 47730 | 150812307 | 150813228 | |
| 47731 | 150856407 | 150857345 | |
| 47732 | 150894034 | 150894910 | |
| 47733 | 150920102 | 150922340 | 120278, 232891 |
| 47734 | 150949406 | 150950446 | 120278 |
| 47735 | 150952907 | 150953927 | 120278 |
| 47736 | 150961462 | 150963504 | 120278 |
| 47737 | 150989450 | 150990700 | 120278 |
| 47738 | 151007548 | 151009336 | 120278 |
| 47739 | 151032596 | 151034061 | 120278 |
| 47740 | 151177546 | 151179419 | |
| 47741 | 151186132 | 151188263 | 120254 |
| 47742 | 151355971 | 151358923 | 120254 |
| 47743 | 151361131 | 151363863 | 120254 |
| 47744 | 151363934 | 151367781 | 120254 |
| 47745 | 151373078 | 151374035 | 120254 |
| 47746 | 151376786 | 151378530 | 232290, 120254 |
| 47747 | 151379582 | 151383262 | 120254 |
| 47748 | 151388036 | 151392987 | 120254 |
| 47749 | 151406286 | 151406759 | 120254 |
| 47750 | 151416941 | 151417729 | 223598, 120254 |
| 47751 | 151439369 | 151440395 | 223598 |
| 47752 | 151457312 | 151458025 | 223598 |
| 47753 | 151644635 | 151648043 | 131016 |
| 47754 | 151710214 | 151713695 | 181472 |
| 47755 | 151767109 | 151768287 | 155906 |
| 47756 | 151772214 | 151774582 | 146476, 155906 |
| 47757 | 151815013 | 151816047 | 120262 |
| 47758 | 151935861 | 151939400 | 207422, 120262 |
| 47759 | 151948166 | 151950132 | |
| 47760 | 151950918 | 151951994 | |
| 47761 | 151954542 | 151955474 | |
| 47762 | 151960772 | 151961311 | |
| 47763 | 151961874 | 151962856 | |
| 47764 | 151972680 | 151974269 | |
| 47765 | 151983041 | 151988087 | 91831 |
| 47766 | 151988163 | 151991707 | 91831 |
| 47767 | 152020636 | 152021391 | 91831 |
| 47768 | 152022525 | 152024209 | 91831 |
| 47769 | 152037462 | 152038183 | 91831 |
| 47770 | 152078372 | 152079467 | 91831 |
| 47771 | 152105660 | 152105993 | 91831 |
| 47772 | 152222209 | 152223158 | 91831 |
| 47773 | 152234937 | 152239775 | 91831 |
| 47774 | 152366993 | 152367621 | 91831 |
| 47775 | 152375386 | 152376162 | 91831 |
| 47776 | 152415363 | 152417334 | 91831 |
| 47777 | 152431869 | 152432942 | 91831 |
| 47778 | 152616473 | 152617719 | 131018 |
| 47779 | 152622307 | 152623708 | 131018 |
| 47780 | 152634431 | 152636337 | 131018 |
| 47781 | 152701026 | 152703135 | 234577, 131018 |
| 47782 | 152896644 | 152897658 | 131018 |
| 47783 | 153067361 | 153067853 | |
| 47784 | 153304181 | 153306289 | 227627, 112029 |
| 47785 | 153322865 | 153324977 | 112031 |
| 47786 | 153487664 | 153488774 | |
| 47787 | 154485494 | 154486004 | 112038, 074706 |
| 47788 | 154613798 | 154614625 | 74706 |
| 47789 | 154688962 | 154689439 | |
| 47790 | 154708372 | 154709116 | 153721 |
| 47791 | 154731357 | 154732451 | 153721 |
| 47792 | 154751456 | 154752375 | 153721 |
| 47793 | 154760929 | 154761998 | 153721 |
| 47794 | 154762118 | 154763522 | 153721 |
| 47795 | 154778748 | 154780131 | 153721 |
| 47796 | 154780671 | 154782721 | 153721 |
| 47797 | 154795621 | 154796976 | 153721 |
| 47798 | 154808267 | 154810505 | 153721 |
| 47799 | 154828359 | 154833334 | 153721 |
| 47800 | 154858557 | 154860149 | |
| 47801 | 155053823 | 155055962 | 213079 |
| 47802 | 155337118 | 155337573 | 146426 |
| 47803 | 155345653 | 155346183 | 146426 |
| 47804 | 155347224 | 155347974 | 146426 |
| 47805 | 155634863 | 155637206 | 29639 |
| 47806 | 156720701 | 156721305 | |
| 47807 | 156742892 | 156743573 | |
| 47808 | 157007283 | 157009921 | |
| 47809 | 157040347 | 157042946 | |
| 47810 | 157073714 | 157074413 | |
| 47811 | 157097503 | 157101930 | 271899, 049618, 270487, |
| | | | 271551 |
| 47812 | 157112368 | 157112980 | 49618 |
| 47813 | 157115782 | 157116842 | 49618 |
| 47814 | 157143845 | 157144403 | 49618 |
| 47815 | 157149034 | 157149931 | 49618 |
| 47816 | 157210427 | 157211079 | 49618 |
| 47817 | 157270123 | 157270915 | 49618 |
| 47818 | 157276625 | 157277540 | 49618 |
| 47819 | 157284634 | 157285876 | 49618 |
| 47820 | 157341541 | 157343768 | 49618 |
| 47821 | 157417346 | 157418219 | 49618 |
| 47822 | 157502705 | 157503238 | 49618 |
| 47823 | 157508125 | 157509158 | 49618 |
| 47824 | 157695588 | 157696715 | |
| 47825 | 157723624 | 157724534 | 215712 |
| 47826 | 157726180 | 157726792 | 215712 |
| 47827 | 157743545 | 157745835 | 271040, 215712 |
| 47828 | 157745962 | 157746755 | |
| 47829 | 157747503 | 157748426 | |
| 47830 | 157800963 | 157803964 | 175048 |
| 47831 | 157845367 | 157846046 | 175048 |
| 47832 | 157849649 | 157851202 | 175048 |
| 47833 | 157855048 | 157856592 | 175048 |
| 47834 | 157881774 | 157882600 | 175048 |
| 47835 | 157935747 | 157936470 | 175048 |
| 47836 | 157942517 | 157942980 | 175048 |
| 47837 | 157950929 | 157953813 | 175048 |
| 47838 | 157956028 | 157957864 | 175048 |
| 47839 | 157960334 | 157961820 | 175048 |
| 47840 | 157977568 | 157978618 | 175048 |
| 47841 | 158000300 | 158001863 | 175048, 219448 |
| 47842 | 158007662 | 158008666 | 175048 |
| 47843 | 158033141 | 158035439 | 175048 |
| 47844 | 158103831 | 158104284 | |
| 47845 | 158184236 | 158185007 | |
| 47846 | 158207921 | 158208749 | |
| 47847 | 158243269 | 158245952 | 130340 |
| 47848 | 158374591 | 158376531 | |
| 47849 | 158401732 | 158404196 | 78269 |
| 47850 | 158414454 | 158414889 | 78269 |
| 47851 | 158429107 | 158429519 | 78269 |
| 47852 | 158446922 | 158448559 | 78269 |
| 47853 | 158454476 | 158456076 | 78269 |
| 47854 | 158460410 | 158464053 | 78269 |
| 47855 | 158464647 | 158465594 | 78269 |
| 47856 | 158480681 | 158483150 | 78269 |
| 47857 | 158488984 | 158490431 | 78269 |
| 47858 | 158588476 | 158590121 | 272047, 122335 |
| 47859 | 158604437 | 158605181 | 272047 |
| 47860 | 158626147 | 158626916 | |
| 47861 | 158652259 | 158654576 | |
| 47862 | 158743610 | 158745214 | 130338 |
| 47863 | 158980405 | 158982316 | 146433 |
| 47864 | 158984487 | 158984905 | 146433 |
| 47865 | 159052436 | 159054520 | 146433 |
| 47866 | 159064653 | 159066334 | 146425 |
| 47867 | 159105731 | 159106465 | 164674 |
| 47868 | 159124736 | 159126027 | 164674 |
| 47869 | 159158346 | 159159693 | 164674 |
| 47870 | 159220062 | 159220793 | 92820 |
| 47871 | 159238996 | 159241192 | 233893, 092820 |
| 47872 | 159242346 | 159242759 | |
| 47873 | 159250663 | 159251527 | |
| 47874 | 159379300 | 159380182 | |
| 47875 | 159415922 | 159416810 | 271913, 130363 |
| 47876 | 159419990 | 159422033 | 130363, 271913 |
| 47877 | 159676324 | 159677514 | 164694 |
| 47878 | 159835620 | 159836207 | |
| 47879 | 160015115 | 160016734 | |
| 47880 | 160097644 | 160099292 | 112096 |
| 47881 | 160099549 | 160100323 | 112096 |
| 47882 | 160100382 | 160101318 | 112096 |
| 47883 | 160101914 | 160104304 | 112096 |
| 47884 | 160104503 | 160105269 | 112096 |
| 47885 | 160105647 | 160106311 | 112096 |
| 47886 | 160106564 | 160107406 | 112096 |
| 47887 | 160109034 | 160110945 | 112096 |
| 47888 | 160111127 | 160116482 | 112096 |
| 47889 | 160119936 | 160124060 | 112096 |
| 47890 | 160135394 | 160140401 | 112096 |
| 47891 | 160146773 | 160149880 | 146457, 112096 |
| 47892 | 160181718 | 160184392 | 112096, 120437 |
| 47893 | 160209279 | 160212625 | 112110, 120438 |
| 47894 | 160228213 | 160228858 | 146453 |
| 47895 | 160321361 | 160322139 | 236823 |
| 47896 | 160335513 | 160336000 | |
| 47897 | 160337633 | 160338924 | |
| 47898 | 160363332 | 160365125 | |
| 47899 | 160388860 | 160391855 | 197081 |
| 47900 | 160395053 | 160395833 | 197081 |
| 47901 | 160401973 | 160403753 | 197081 |
| 47902 | 160451162 | 160451584 | 197081 |
| 47903 | 160462402 | 160465526 | 197081 |
| 47904 | 160479500 | 160480219 | 197081 |
| 47905 | 160486106 | 160486824 | 197081 |
| 47906 | 160513610 | 160514486 | 213073, 197081 |
| 47907 | 160542478 | 160546643 | 175003 |
| 47908 | 160558123 | 160558891 | 175003 |
| 47909 | 160558989 | 160561248 | 175003 |
| 47910 | 160561924 | 160563930 | 175003 |
| 47911 | 160577799 | 160578397 | 175003 |
| 47912 | 160581306 | 160582007 | |
| 47913 | 160584260 | 160585440 | 216516 |
| 47914 | 160619478 | 160620224 | 112499 |
| 47915 | 160658100 | 160661394 | 112499 |
| 47916 | 160668490 | 160670760 | 112499 |
| 47917 | 160673698 | 160677950 | 112499 |
| 47918 | 160768285 | 160772057 | 146477 |
| 47919 | 160798483 | 160802694 | 146477 |
| 47920 | 160806890 | 160814175 | 146477 |
| 47921 | 160819597 | 160822007 | 146477 |
| 47922 | 160848056 | 160848726 | 146477 |
| 47923 | 160928324 | 160928965 | 213071 |
| 47924 | 160940831 | 160942131 | |
| 47925 | 161339223 | 161341350 | 224371 |
| 47926 | 161411884 | 161414174 | 085511, 272841 |
| 47927 | 161583695 | 161584227 | 26652 |
| 47928 | 161855844 | 161856434 | 185345 |
| 47929 | 162058651 | 162059161 | 185345 |
| 47930 | 162171468 | 162172837 | 185345 |
| 47931 | 162342726 | 162346341 | 185345 |
| 47932 | 162479043 | 162480344 | 185345 |
| 47933 | 162519830 | 162521598 | 185345 |
| 47934 | 162671154 | 162671768 | 185345 |
| 47935 | 162794121 | 162794768 | 185345 |
| 47936 | 163095352 | 163095892 | 185345 |
| 47937 | 163147720 | 163149206 | 112530, 185345 |
| 47938 | 163275060 | 163275874 | 112530 |
| 47939 | 163277778 | 163278577 | 112530 |
| 47940 | 163303593 | 163304836 | 112530 |
| 47941 | 163309071 | 163310074 | 112530 |
| 47942 | 163601428 | 163603355 | 225683, 112530 |
| 47943 | 163617148 | 163618109 | 112530 |
| 47944 | 163745227 | 163746876 | |
| 47945 | 163826450 | 163827666 | |
| 47946 | 163829512 | 163830092 | |
| 47947 | 163833327 | 163838417 | 112531, 270419 |
| 47948 | 163878830 | 163879306 | 112531 |
| 47949 | 163950251 | 163951228 | 112531 |
| 47950 | 163954336 | 163954972 | 112531 |
| 47951 | 163960109 | 163960754 | 112531 |
| 47952 | 164004639 | 164005142 | |
| 47953 | 164166770 | 164167653 | 235538 |
| 47954 | 164519722 | 164520403 | |
| 47955 | 164527998 | 164531247 | 230627 |
| 47956 | 166254417 | 166255279 | 112541 |
| 47957 | 166679356 | 166680415 | |
| 47958 | 166700253 | 166701143 | |
| 47959 | 166715105 | 166716035 | |
| 47960 | 166720984 | 166722343 | 176381 |
| 47961 | 166743959 | 166744589 | 198818 |
| 47962 | 166754930 | 166757303 | 198818 |
| 47963 | 166760791 | 166762466 | |
| 47964 | 166773670 | 166775081 | |
| 47965 | 166779050 | 166779961 | 60762 |
| 47966 | 166793203 | 166793865 | 60762 |
| 47967 | 166794434 | 166797779 | 261420, 060762 |
| 47968 | 166800974 | 166801946 | |
| 47969 | 166969708 | 166971439 | 71242 |
| 47970 | 167033408 | 167034003 | 71242 |
| 47971 | 167039662 | 167041973 | 71242 |
| 47972 | 167091929 | 167092462 | 71242 |
| 47973 | 167368963 | 167370137 | 249141, 026297 |
| 47974 | 167412417 | 167414217 | 213066, 272980, 227598 |
| 47975 | 167444781 | 167445911 | 213066, 272980 |
| 47976 | 167624379 | 167624874 | |
| 47977 | 167682227 | 167683187 | |
| 47978 | 167690563 | 167691292 | 112494 |
| 47979 | 167698525 | 167699855 | 112494 |
| 47980 | 167721575 | 167723241 | 112494 |
| 47981 | 167754853 | 167756208 | 120440 |
| 47982 | 167764109 | 167765260 | 120440 |
| 47983 | 167834767 | 167835312 | |
| 47984 | 167904835 | 167905301 | |
| 47985 | 167907531 | 167910139 | |
| 47986 | 167914093 | 167914793 | |
| 47987 | 168061705 | 168062842 | |
| 47988 | 168068345 | 168069838 | 272549 |
| 47989 | 168071181 | 168072102 | 272549 |
| 47990 | 168074579 | 168075091 | 272549 |
| 47991 | 168076346 | 168077484 | 272549 |
| 47992 | 168078951 | 168079709 | 272549 |
| 47993 | 168082570 | 168084903 | 203688 |
| 47994 | 168134516 | 168135215 | |
| 47995 | 168135603 | 168136695 | |
| 47996 | 168151495 | 168152081 | |
| 47997 | 168161622 | 168162607 | |
| 47998 | 168162831 | 168163523 | |
| 47999 | 168179099 | 168179719 | |
| 48000 | 168191018 | 168193136 | 146521 |
| 48001 | 168196687 | 168198322 | 146521, 269155 |
| 48002 | 168206239 | 168207638 | |
| 48003 | 168225747 | 168227570 | 198221 |
| 48004 | 168227715 | 168228994 | 130396 |
| 48005 | 168250346 | 168251523 | 130396 |
| 48006 | 168332542 | 168335472 | 130396 |
| 48007 | 168338429 | 168339453 | 130396 |
| 48008 | 168357270 | 168358577 | 130396 |
| 48009 | 168363426 | 168367120 | 130396 |
| 48010 | 168408049 | 168409279 | 125337 |
| 48011 | 168424607 | 168425793 | 125337 |
| 48012 | 168517562 | 168518330 | |
| 48013 | 168596757 | 168597964 | 234768 |
| 48014 | 168600835 | 168602511 | 234768 |
| 48015 | 168647920 | 168649020 | 234647 |
| 48016 | 168661855 | 168663324 | 234647 |
| 48017 | 168664774 | 168666269 | |
| 48018 | 168674857 | 168675624 | |
| 48019 | 168702331 | 168703118 | 224417, 164488 |
| 48020 | 168726976 | 168727482 | |
| 48021 | 169284577 | 169285225 | |
| 48022 | 169612858 | 169613646 | 226445 |
| 48023 | 169692140 | 169693112 | |
| 48024 | 169868951 | 169869716 | 184465 |
| 48025 | 169873118 | 169874206 | 184465 |
| 48026 | 169929187 | 169930805 | 184465 |
| 48027 | 170101036 | 170103595 | 185127, 184465 |
| 48028 | 170122660 | 170125759 | 232640, 227704, 130024 |
| 48029 | 170150681 | 170153050 | 130023, 184786 |
| 48030 | 170189288 | 170191409 | 231690, 229214 |
| 48031 | 170199604 | 170201923 | 231690 |
| 48032 | 170231542 | 170233522 | |
| 48033 | 170364977 | 170365309 | |
| 48034 | 170444918 | 170446022 | |
| 48035 | 170459371 | 170461497 | |
| 48036 | 170493513 | 170495758 | 273100 |
| 48037 | 170534616 | 170535973 | |
| 48038 | 170567966 | 170568849 | 230960 |
| 48039 | 170574613 | 170575517 | |
| 48040 | 170615076 | 170616922 | 112584 |
| 48041 | 170653986 | 170654584 | 112584 |
| 48042 | 170861299 | 170864485 | 112592, 008018 |
| 48043 | 170892799 | 170894592 | 71994 |

The locations of ODSCs on Chromosome 7 are shown in Table 49.

TABLE 49
ODSC Locations on Chromosome 7

| SEQ ID NO. | Start | End | Overlapping Gene(s) |
|---|---|---|---|
| 48044 | 141973 | 143586 | 242474 |
| 48045 | 191831 | 195429 | 177706, 240093 |
| 48046 | 214225 | 215251 | 177706 |
| 48047 | 358806 | 359344 | |
| 48048 | 366736 | 367568 | |
| 48049 | 372422 | 373497 | |
| 48050 | 381500 | 382737 | |
| 48051 | 514155 | 514874 | |
| 48052 | 543086 | 544975 | 197461 |
| 48053 | 556044 | 557918 | 197461 |
| 48054 | 557999 | 559308 | 197461 |
| 48055 | 765545 | 767792 | 164818, 188191 |
| 48056 | 771763 | 772602 | 164818 |
| 48057 | 852122 | 853937 | |
| 48058 | 855770 | 857604 | 164828 |
| 48059 | 861114 | 861794 | 164828 |
| 48060 | 895567 | 896137 | 164828 |
| 48061 | 900441 | 901536 | 164828 |
| 48062 | 905776 | 907802 | 164828 |
| 48063 | 915181 | 918045 | 239857, 164828 |
| 48064 | 1013643 | 1015463 | 240230 |
| 48065 | 1032429 | 1033316 | |
| 48066 | 1067191 | 1068851 | 225146, 146540 |
| 48067 | 1082865 | 1085347 | 164849, 225146, 146540 |
| 48068 | 1126076 | 1129006 | 146540, 164850 |
| 48069 | 1131946 | 1133974 | 164850, 146540 |
| 48070 | 1135211 | 1136779 | 146540 |
| 48071 | 1141134 | 1142301 | 146540 |
| 48072 | 1177177 | 1178262 | 146540 |
| 48073 | 1195023 | 1200900 | 229043, 178381 |
| 48074 | 1215448 | 1217048 | |
| 48075 | 1497786 | 1499368 | 164877 |
| 48076 | 1499437 | 1501972 | 225981 |
| 48077 | 1506249 | 1506829 | 273230 |
| 48078 | 1542884 | 1544484 | 164880 |
| 48079 | 1546434 | 1546999 | |
| 48080 | 1556218 | 1556673 | |
| 48081 | 1559662 | 1560808 | |
| 48082 | 1569976 | 1574620 | 198517 |
| 48083 | 1576082 | 1578019 | 198517 |
| 48084 | 1581828 | 1583263 | 164855, 198517 |
| 48085 | 1594060 | 1596487 | 164855 |
| 48086 | 1608915 | 1610787 | 230487, 157778 |
| 48087 | 1704068 | 1705291 | |
| 48088 | 1705359 | 1705869 | |
| 48089 | 1706951 | 1708714 | |
| 48090 | 1709562 | 1710738 | |
| 48091 | 1753032 | 1754463 | 225968 |
| 48092 | 1760611 | 1761226 | 225968 |
| 48093 | 1881835 | 1882326 | 002822, 176349 |
| 48094 | 1911745 | 1912868 | 002822 |
| 48095 | 1935159 | 1936787 | 002822 |
| 48096 | 1962782 | 1963768 | 002822 |
| 48097 | 1968154 | 1968656 | 002822 |
| 48098 | 2271750 | 2273138 | 002822 |
| 48099 | 2281001 | 2282208 | 106268, 122687 |
| 48100 | 2353106 | 2354635 | 106266 |
| 48101 | 2393055 | 2395784 | 106263, 106266 |
| 48102 | 2442538 | 2444310 | 136213 |
| 48103 | 2594560 | 2595738 | 106009 |
| 48104 | 2598190 | 2599539 | 106012 |
| 48105 | 2669268 | 2670610 | |
| 48106 | 2671382 | 2673470 | 136295 |
| 48107 | 2675346 | 2676657 | 136295 |
| 48108 | 2676708 | 2679334 | 136295 |
| 48109 | 2680515 | 2681811 | 136295 |
| 48110 | 2701582 | 2704288 | 136295 |
| 48111 | 2733318 | 2734173 | 174945 |
| 48112 | 2796875 | 2798741 | 174945, 146535 |
| 48113 | 2850231 | 2851886 | 146535 |
| 48114 | 2858544 | 2862456 | 146535 |
| 48115 | 2882898 | 2884525 | 146535 |
| 48116 | 2903070 | 2904183 | |
| 48117 | 3340213 | 3341104 | 146555, 236708 |
| 48118 | 4228311 | 4229804 | 146555 |
| 48119 | 4318786 | 4319468 | |
| 48120 | 4671211 | 4671832 | |
| 48121 | 4681149 | 4682681 | |
| 48122 | 4721401 | 4724280 | 164916 |
| 48123 | 4755069 | 4755382 | 164916 |
| 48124 | 4764176 | 4766022 | 164916 |
| 48125 | 4778193 | 4779711 | 164916 |
| 48126 | 4784377 | 4786316 | 164916 |
| 48127 | 4813975 | 4816392 | 242802 |
| 48128 | 5013483 | 5014333 | 196204 |
| 48129 | 5085036 | 5086056 | 146587, 272968 |
| 48130 | 5216561 | 5217161 | |
| 48131 | 5229427 | 5230734 | 157954 |
| 48132 | 5258586 | 5258995 | 157954 |
| 48133 | 5279704 | 5280836 | |
| 48134 | 5436028 | 5437883 | 182095 |
| 48135 | 5450851 | 5451214 | 182095 |
| 48136 | 5458068 | 5470621 | 272953, 241269, 188365, |
| | | | 273084, 234432, 182095 |
| 48137 | 5526371 | 5526995 | 155034 |
| 48138 | 5534094 | 5536072 | 207973, 155034 |
| 48139 | 5552550 | 5554807 | 155034 |
| 48140 | 5560877 | 5561414 | |
| 48141 | 5562625 | 5563377 | |
| 48142 | 5564773 | 5566981 | 075624 |
| 48143 | 5568084 | 5572198 | 075624 |
| 48144 | 5592298 | 5592875 | 075624 |
| 48145 | 5594268 | 5598157 | 272719, 075624 |
| 48146 | 5598194 | 5599009 | 075624 |
| 48147 | 5730846 | 5734390 | 011275 |
| 48148 | 5740559 | 5741283 | 011275 |
| 48149 | 5820479 | 5821541 | 011275 |
| 48150 | 5861982 | 5862993 | 235944 |
| 48151 | 5908501 | 5909033 | |
| 48152 | 6048009 | 6049716 | 106305, 122512 |
| 48153 | 6051989 | 6053000 | 106305 |
| 48154 | 6095001 | 6095472 | 086232 |
| 48155 | 6097258 | 6099042 | 086232 |
| 48156 | 6116813 | 6117445 | |
| 48157 | 6120146 | 6121611 | 231704 |
| 48158 | 6143635 | 6146400 | 106346 |
| 48159 | 6311524 | 6312708 | 008256 |
| 48160 | 6322686 | 6323437 | |
| 48161 | 6345997 | 6346618 | |
| 48162 | 6388050 | 6389014 | 178397 |
| 48163 | 6413693 | 6415813 | 136238 |
| 48164 | 6416270 | 6417474 | 136238 |
| 48165 | 6424129 | 6426116 | 136238 |
| 48166 | 6458907 | 6459443 | 164535 |
| 48167 | 6483006 | 6483803 | 164535 |
| 48168 | 6486581 | 6488424 | 136240, 164535 |
| 48169 | 6521822 | 6524291 | 269781, 136240, 164535 |
| 48170 | 6616736 | 6618612 | 136247, 232581 |
| 48171 | 6629267 | 6630404 | 146576 |
| 48172 | 6676032 | 6677962 | 205903 |
| 48173 | 6745474 | 6747131 | 164631, 228010 |
| 48174 | 6749316 | 6749960 | 187953 |
| 48175 | 6768997 | 6770176 | 187953 |
| 48176 | 7138973 | 7139665 | |
| 48177 | 7215959 | 7217726 | 244167, 106392 |
| 48178 | 7220791 | 7223488 | 106392 |
| 48179 | 7477889 | 7479785 | 215018 |
| 48180 | 7529131 | 7533743 | 215018 |
| 48181 | 7603239 | 7604255 | 272894 |
| 48182 | 7605861 | 7607493 | 164654, 272894 |
| 48183 | 7679285 | 7681520 | 219545, 106399 |
| 48184 | 7734830 | 7737628 | 219545, 106399 |
| 48185 | 7747278 | 7748095 | 219545, 106399 |
| 48186 | 7790222 | 7791857 | 219545 |
| 48187 | 7800694 | 7802597 | 219545 |
| 48188 | 7838671 | 7839411 | 219545 |
| 48189 | 7847336 | 7848278 | 219545 |
| 48190 | 7888473 | 7889024 | 219545 |
| 48191 | 7900751 | 7901367 | 219545 |
| 48192 | 8007945 | 8010082 | 106415, 233108, 219545 |
| 48193 | 8035927 | 8036796 | 219545, 106415 |
| 48194 | 8180727 | 8181185 | 003147 |
| 48195 | 8229750 | 8230964 | 003147 |
| 48196 | 8300478 | 8302502 | 229970, 244239, 003147 |
| 48197 | 8962315 | 8963146 | |
| 48198 | 10276853 | 10277388 | |
| 48199 | 10694505 | 10695125 | |
| 48200 | 10978894 | 10981118 | 271185, 189043 |
| 48201 | 11012464 | 11014445 | 106443 |
| 48202 | 11121599 | 11123189 | 106443 |
| 48203 | 11232750 | 11234602 | 230333 |
| 48204 | 11277166 | 11277977 | 230333 |
| 48205 | 11287571 | 11288292 | 230333 |
| 48206 | 11475594 | 11476666 | 230333, 005108 |
| 48207 | 11501268 | 11502099 | 230333, 005108 |
| 48208 | 11609812 | 11610830 | 005108 |
| 48209 | 11660405 | 11661164 | 005108 |
| 48210 | 11745598 | 11747597 | 005108 |
| 48211 | 11870200 | 11872037 | 005108 |
| 48212 | 12249692 | 12252143 | 106460 |
| 48213 | 12714400 | 12714995 | |
| 48214 | 12725562 | 12727735 | 122644, 271253 |
| 48215 | 12759038 | 12760186 | |
| 48216 | 12962673 | 12964641 | |
| 48217 | 12981854 | 12982680 | |
| 48218 | 13017766 | 13019661 | |
| 48219 | 13028874 | 13029427 | |
| 48220 | 13056510 | 13057403 | |
| 48221 | 13952573 | 13953049 | 006468 |
| 48222 | 14028253 | 14029816 | 006468 |
| 48223 | 14030322 | 14030946 | 006468 |
| 48224 | 14231404 | 14231971 | 233167, 136267 |
| 48225 | 14245916 | 14248234 | 136267 |
| 48226 | 14257458 | 14259560 | 136267 |
| 48227 | 14276059 | 14278345 | 136267 |
| 48228 | 14291794 | 14292641 | 136267 |
| 48229 | 14342926 | 14343613 | 136267 |
| 48230 | 14375782 | 14376498 | 136267 |
| 48231 | 14535183 | 14535964 | 136267 |
| 48232 | 14675103 | 14676143 | 136267 |
| 48233 | 14753623 | 14755116 | 136267 |
| 48234 | 15039046 | 15040267 | |
| 48235 | 15344473 | 15345532 | 187546 |
| 48236 | 15415657 | 15417493 | 187546 |
| 48237 | 15593542 | 15595761 | 187546 |
| 48238 | 15596225 | 15598102 | 187546 |
| 48239 | 15600346 | 15602119 | 187546 |
| 48240 | 15620059 | 15621672 | |
| 48241 | 15724781 | 15726123 | 106511 |
| 48242 | 16065773 | 16066520 | |
| 48243 | 16459721 | 16462181 | 214960 |
| 48244 | 16684112 | 16686795 | 136261, 106524 |
| 48245 | 16700334 | 16701112 | 136261 |
| 48246 | 16792846 | 16793717 | 106537 |
| 48247 | 16967534 | 16968134 | |
| 48248 | 17017861 | 17019156 | |
| 48249 | 17029369 | 17030101 | |
| 48250 | 17060687 | 17062038 | |
| 48251 | 17066818 | 17067493 | |
| 48252 | 17125984 | 17126371 | |
| 48253 | 17137707 | 17140272 | |
| 48254 | 17169862 | 17170920 | |
| 48255 | 17181710 | 17185304 | |
| 48256 | 17224412 | 17224899 | |
| 48257 | 17251630 | 17252444 | |
| 48258 | 17278897 | 17280310 | |
| 48259 | 17314783 | 17316558 | |
| 48260 | 17337028 | 17339985 | 106546, 237773 |
| 48261 | 17343819 | 17345498 | 106546 |
| 48262 | 17346007 | 17348400 | 106546 |
| 48263 | 17410459 | 17412458 | |
| 48264 | 17442429 | 17443101 | 236318 |
| 48265 | 17445320 | 17447397 | 236318 |
| 48266 | 17447665 | 17448875 | 236318 |
| 48267 | 17484152 | 17484754 | 236039, 236318 |
| 48268 | 17545616 | 17546311 | 226598 |
| 48269 | 17562963 | 17565009 | 226598 |
| 48270 | 17582027 | 17582723 | 226598 |
| 48271 | 17639249 | 17640344 | |
| 48272 | 17658075 | 17658791 | |
| 48273 | 17672606 | 17673029 | |
| 48274 | 17719454 | 17721053 | 228675 |
| 48275 | 17854874 | 17855389 | 071189 |
| 48276 | 17978625 | 17981145 | 071189 |
| 48277 | 18002813 | 18003393 | |
| 48278 | 18547894 | 18549786 | 048052 |
| 48279 | 19747769 | 19749044 | 105849 |
| 48280 | 19997706 | 19998637 | 243004 |
| 48281 | 20009030 | 20010479 | 243004 |
| 48282 | 20777050 | 20778926 | 004846 |
| 48283 | 20837348 | 20838347 | |
| 48284 | 21104309 | 21105255 | |
| 48285 | 21290771 | 21291751 | |
| 48286 | 21405845 | 21406224 | |
| 48287 | 21447579 | 21448211 | |
| 48288 | 21466430 | 21468524 | 105866 |
| 48289 | 21605189 | 21606219 | 105877 |
| 48290 | 21618814 | 21620873 | 105877 |
| 48291 | 21650445 | 21651449 | 105877 |
| 48292 | 21656048 | 21658906 | 105877 |
| 48293 | 21678988 | 21679511 | 105877 |
| 48294 | 21797776 | 21798392 | 105877 |
| 48295 | 21984096 | 21986302 | 164649 |
| 48296 | 22000393 | 22000858 | |
| 48297 | 22121963 | 22123819 | |
| 48298 | 22255710 | 22256358 | 136237 |
| 48299 | 22280639 | 22281343 | 136237 |
| 48300 | 22379284 | 22381575 | 136237 |
| 48301 | 22383111 | 22387562 | 136237 |
| 48302 | 22390089 | 22391866 | 136237 |
| 48303 | 22395580 | 22398220 | 136237 |
| 48304 | 22398308 | 22399606 | |
| 48305 | 22400891 | 22401905 | |
| 48306 | 22417662 | 22418802 | |
| 48307 | 22616845 | 22617779 | 105889, 225541 |
| 48308 | 22825856 | 22826526 | |
| 48309 | 22861391 | 22863344 | 196683 |
| 48310 | 22893394 | 22895436 | 228649 |
| 48311 | 22899111 | 22899916 | 228649 |
| 48312 | 22904787 | 22905455 | 226329 |
| 48313 | 23052424 | 23054146 | 122591 |
| 48314 | 23144526 | 23146390 | 122550, 230658 |
| 48315 | 23220896 | 23222492 | 136243, 214871 |
| 48316 | 23312566 | 23313669 | 136235 |
| 48317 | 23337392 | 23339996 | 156928 |
| 48318 | 23372663 | 23374752 | 136231 |
| 48319 | 23396428 | 23397997 | 136231 |
| 48320 | 23570328 | 23572314 | 164548 |
| 48321 | 23636489 | 23638325 | 169193 |
| 48322 | 23662062 | 23662516 | 227436, 169193 |
| 48323 | 23719463 | 23721193 | 234286, 188732 |
| 48324 | 23799604 | 23800176 | 196335 |
| 48325 | 23996227 | 23997199 | |
| 48326 | 24114284 | 24115325 | |
| 48327 | 24495379 | 24497736 | |
| 48328 | 24612068 | 24614405 | 105926 |
| 48329 | 24708082 | 24709799 | 105926 |
| 48330 | 24796343 | 24797935 | 105928 |
| 48331 | 24826479 | 24827051 | |
| 48332 | 24859740 | 24861557 | 070882 |
| 48333 | 24865662 | 24866433 | 070882 |
| 48334 | 24944442 | 24944911 | 070882 |
| 48335 | 25018973 | 25021433 | 070882 |
| 48336 | 25064484 | 25065968 | |
| 48337 | 25067549 | 25069154 | |
| 48338 | 25163762 | 25165954 | 172115 |
| 48339 | 25219269 | 25220575 | 153790 |
| 48340 | 25353846 | 25355228 | |
| 48341 | 25469171 | 25470027 | |
| 48342 | 25607996 | 25608882 | |
| 48343 | 25637395 | 25638132 | 223561 |
| 48344 | 25888726 | 25890146 | |
| 48345 | 25989285 | 25992903 | 199085 |
| 48346 | 26195321 | 26196477 | 050344 |
| 48347 | 26198635 | 26199303 | 050344 |
| 48348 | 26237587 | 26242554 | 122565, 122566 |
| 48349 | 26330967 | 26332295 | 086300 |
| 48350 | 26344470 | 26345842 | 086300 |
| 48351 | 26346139 | 26347199 | 086300 |
| 48352 | 26382796 | 26383749 | 086300 |
| 48353 | 26385077 | 26389246 | 086300 |
| 48354 | 26406337 | 26407705 | 086300 |
| 48355 | 26725948 | 26726654 | 005020 |
| 48356 | 26738226 | 26740347 | 005020 |
| 48357 | 26745116 | 26745889 | 005020 |
| 48358 | 26903475 | 26905142 | 005020 |
| 48359 | 27161759 | 27162471 | 253552, 105997 |
| 48360 | 27249867 | 27250769 | |
| 48361 | 27317098 | 27317841 | |
| 48362 | 27385873 | 27386928 | |
| 48363 | 27403666 | 27405039 | 224322 |
| 48364 | 27458899 | 27459853 | |
| 48365 | 27644752 | 27647303 | 106049 |
| 48366 | 27656342 | 27658132 | 106049 |
| 48367 | 27680206 | 27682489 | 106049 |
| 48368 | 27701155 | 27704183 | 106049 |
| 48369 | 27716678 | 27719282 | |
| 48370 | 27774701 | 27775287 | 229893 |
| 48371 | 27779056 | 27781003 | 229893, 106052 |
| 48372 | 27784219 | 27785353 | 106052 |
| 48373 | 27913380 | 27914301 | 153814 |
| 48374 | 27936898 | 27937697 | 153814 |
| 48375 | 28044899 | 28045693 | 153814 |
| 48376 | 28073866 | 28074811 | 153814 |
| 48377 | 28075700 | 28078153 | 153814 |
| 48378 | 28123249 | 28123685 | 153814 |
| 48379 | 28188720 | 28189924 | 153814 |
| 48380 | 28195328 | 28196110 | 153814 |
| 48381 | 28208454 | 28209366 | 153814 |
| 48382 | 28218574 | 28220749 | 234336, 153814 |
| 48383 | 28269522 | 28270334 | 234336 |
| 48384 | 28447423 | 28449926 | 146592 |
| 48385 | 28725127 | 28726292 | 146592 |
| 48386 | 28894897 | 28897323 | |
| 48387 | 29139602 | 29140701 | 106066 |
| 48388 | 29233416 | 29235684 | 106066, 106069 |
| 48389 | 29243899 | 29244922 | 106069, 235669 |
| 48390 | 29253577 | 29255012 | 106069 |
| 48391 | 29267969 | 29268863 | 106069 |
| 48392 | 29270351 | 29271152 | 106069 |
| 48393 | 29286653 | 29287830 | 106069 |
| 48394 | 29288273 | 29289455 | 106069 |
| 48395 | 29294358 | 29297196 | 106069 |
| 48396 | 29304313 | 29305104 | 106069 |
| 48397 | 29310261 | 29311670 | 106069 |
| 48398 | 29313554 | 29314805 | 106069 |
| 48399 | 29326344 | 29327406 | 106069 |
| 48400 | 29331233 | 29332551 | 106069 |
| 48401 | 29367518 | 29369344 | 106069 |
| 48402 | 29369643 | 29370452 | 106069 |
| 48403 | 29414994 | 29416768 | 106069 |
| 48404 | 29452343 | 29453210 | 106069 |
| 48405 | 29473343 | 29475690 | 106069 |
| 48406 | 29487372 | 29488355 | 106069 |
| 48407 | 29488956 | 29490270 | 106069 |
| 48408 | 29502948 | 29503840 | 106069 |
| 48409 | 29510575 | 29512354 | 106069 |
| 48410 | 29522740 | 29523592 | 106069 |
| 48411 | 29527507 | 29529334 | 106069 |
| 48412 | 29533887 | 29534411 | 106069 |
| 48413 | 29535782 | 29536733 | 106069 |
| 48414 | 29845588 | 29847678 | 122574 |
| 48415 | 29904778 | 29905746 | 122574 |
| 48416 | 30027347 | 30027983 | 136193 |
| 48417 | 30065424 | 30069155 | 106086, 106080 |
| 48418 | 30085754 | 30086438 | 106086 |
| 48419 | 30107635 | 30108225 | 106086 |
| 48420 | 30130988 | 30131461 | 106086 |
| 48421 | 30161074 | 30161813 | 106086 |
| 48422 | 30174177 | 30175943 | 180354 |
| 48423 | 30208789 | 30212352 | |
| 48424 | 30278285 | 30279097 | |
| 48425 | 30292655 | 30293459 | |
| 48426 | 30293641 | 30296088 | |
| 48427 | 30302317 | 30303341 | |
| 48428 | 30323626 | 30325261 | 180233 |
| 48429 | 30325362 | 30325932 | 180233 |
| 48430 | 30417846 | 30419187 | 180233 |
| 48431 | 30452438 | 30453187 | |
| 48432 | 30517735 | 30519424 | 106100 |
| 48433 | 30543722 | 30545487 | 006625 |
| 48434 | 30632964 | 30636493 | 106105, 196295 |
| 48435 | 30809691 | 30811962 | 106125, 254959 |
| 48436 | 30836837 | 30837309 | 254959, 229263, 106125 |
| 48437 | 30906408 | 30907668 | 240583, 254959, 250424, |
| | | | 106125 |
| 48438 | 30935605 | 30936013 | 240583, 250424 |
| 48439 | 30936912 | 30937477 | 240583, 250424 |
| 48440 | 31014230 | 31014898 | 106128 |
| 48441 | 31357586 | 31358774 | |
| 48442 | 31375209 | 31376854 | |
| 48443 | 31504194 | 31504557 | |
| 48444 | 32529272 | 32530865 | 106355 |
| 48445 | 32534230 | 32536080 | 105778, 106355 |
| 48446 | 32555306 | 32555998 | 105778 |
| 48447 | 32707919 | 32708487 | 229358, 105778 |
| 48448 | 32709410 | 32710632 | 229358, 105778 |
| 48449 | 32929785 | 32933045 | 105778, 170852 |
| 48450 | 32981432 | 32983146 | 205763, 105778 |
| 48451 | 32996662 | 32998123 | 122642, 105778 |
| 48452 | 33073641 | 33075286 | 105778, 122643 |
| 48453 | 33079002 | 33081017 | 122643 |
| 48454 | 33083516 | 33084410 | 122643 |
| 48455 | 33101477 | 33103427 | 122643 |
| 48456 | 33148110 | 33149372 | 164610 |
| 48457 | 33168252 | 33169419 | 228015, 122507 |
| 48458 | 33716472 | 33719633 | |
| 48459 | 33972332 | 33974447 | 164619 |
| 48460 | 34037289 | 34038921 | 164619 |
| 48461 | 34075427 | 34076565 | 164619 |
| 48462 | 34103163 | 34105602 | 164619 |
| 48463 | 34127634 | 34128298 | 164619 |
| 48464 | 34147511 | 34148111 | 164619 |
| 48465 | 34149459 | 34150004 | 164619 |
| 48466 | 34787208 | 34789405 | 197085, 187258 |
| 48467 | 34798959 | 34799619 | 197085, 187258 |
| 48468 | 34978679 | 34979147 | 173852 |
| 48469 | 35054947 | 35055767 | 173852 |
| 48470 | 35075108 | 35077955 | 173852 |
| 48471 | 35440815 | 35441682 | |
| 48472 | 35575690 | 35577539 | |
| 48473 | 35596168 | 35597360 | |
| 48474 | 35686255 | 35688630 | 122557 |
| 48475 | 35733526 | 35735630 | 271122, 122557 |
| 48476 | 35736592 | 35737510 | 271122 |
| 48477 | 35772177 | 35772911 | 227544 |
| 48478 | 35775605 | 35776800 | |
| 48479 | 35839379 | 35842292 | 122545, 228878 |
| 48480 | 35870798 | 35871286 | 122545 |
| 48481 | 35891716 | 35892175 | 122545 |
| 48482 | 36027280 | 36028625 | |
| 48483 | 36033261 | 36033924 | |
| 48484 | 36119035 | 36119851 | 205745 |
| 48485 | 36154974 | 36159436 | |
| 48486 | 36167468 | 36169735 | |
| 48487 | 36191111 | 36195151 | 122547 |
| 48488 | 36195386 | 36196422 | 122547 |
| 48489 | 36196681 | 36198572 | 122547 |
| 48490 | 36201578 | 36202011 | 122547 |
| 48491 | 36204631 | 36205158 | 122547 |
| 48492 | 36245085 | 36248753 | 122547 |
| 48493 | 36272080 | 36273518 | 122547 |
| 48494 | 36312043 | 36313852 | 122547 |
| 48495 | 36320400 | 36321903 | 122547 |
| 48496 | 36329135 | 36332772 | 122547 |
| 48497 | 36342772 | 36344012 | |
| 48498 | 36406029 | 36407231 | 164542 |
| 48499 | 36428982 | 36430331 | 011426, 164542 |
| 48500 | 36500277 | 36501076 | |
| 48501 | 36510526 | 36511489 | 237400 |
| 48502 | 36527237 | 36527845 | 237400 |
| 48503 | 36566302 | 36567313 | 136250 |
| 48504 | 36640541 | 36641362 | 136250 |
| 48505 | 36644053 | 36645334 | 136250 |
| 48506 | 36690974 | 36691674 | 136250 |
| 48507 | 36697924 | 36699223 | 136250 |
| 48508 | 36725622 | 36726623 | 136250 |
| 48509 | 36749977 | 36751411 | 136250 |
| 48510 | 36782370 | 36782892 | |
| 48511 | 36784819 | 36790624 | 229424 |
| 48512 | 36799644 | 36802161 | 229424 |
| 48513 | 36820906 | 36821503 | 235728 |
| 48514 | 36834054 | 36837272 | |
| 48515 | 36860011 | 36861214 | |
| 48516 | 36918545 | 36919151 | 155849 |
| 48517 | 36936063 | 36937490 | 155849 |
| 48518 | 36937510 | 36938248 | 155849 |
| 48519 | 36989249 | 36989728 | 155849 |
| 48520 | 37010536 | 37012215 | 155849 |
| 48521 | 37026214 | 37027200 | 155849 |
| 48522 | 37055148 | 37056110 | 155849 |
| 48523 | 37070464 | 37072893 | 155849 |
| 48524 | 37113832 | 37114405 | 155849 |
| 48525 | 37129444 | 37130551 | 155849 |
| 48526 | 37231340 | 37231846 | 155849 |
| 48527 | 37336990 | 37337557 | 155849 |
| 48528 | 37359833 | 37360959 | 155849 |
| 48529 | 37393545 | 37394914 | 155849 |
| 48530 | 37407648 | 37408620 | 155849 |
| 48531 | 37447616 | 37449310 | 155849 |
| 48532 | 37452752 | 37456125 | 155849 |
| 48533 | 37461435 | 37461919 | 155849 |
| 48534 | 37487112 | 37489246 | 155849 |
| 48535 | 37527529 | 37530176 | |
| 48536 | 37940752 | 37942636 | 086289 |
| 48537 | 37955907 | 37957087 | 106483, 086289 |
| 48538 | 37959845 | 37961893 | 106483, 086289 |
| 48539 | 37982239 | 37983794 | 106483, 086289 |
| 48540 | 38217332 | 38218626 | 010270 |
| 48541 | 38623778 | 38624608 | 078053 |
| 48542 | 38742654 | 38743052 | |
| 48543 | 38783831 | 38785438 | 006715 |
| 48544 | 38872823 | 38873362 | 006715 |
| 48545 | 38902921 | 38905423 | 006715 |
| 48546 | 38912716 | 38913828 | 006715 |
| 48547 | 38947961 | 38949066 | 006715 |
| 48548 | 38981947 | 38982981 | |
| 48549 | 39445786 | 39446480 | 224122, 106536 |
| 48550 | 39453506 | 39454453 | 106536 |
| 48551 | 39504367 | 39505395 | 106536 |
| 48552 | 39605061 | 39606650 | 241127, 227172 |
| 48553 | 39662113 | 39664789 | 006451 |
| 48554 | 39667473 | 39667919 | 006451 |
| 48555 | 39772521 | 39773800 | 188185 |
| 48556 | 39935910 | 39936573 | |
| 48557 | 39959066 | 39959906 | |
| 48558 | 39970425 | 39971286 | |
| 48559 | 39987798 | 39992319 | 065883, 259826 |
| 48560 | 40133502 | 40133924 | 065883 |
| 48561 | 40134685 | 40135611 | 065883 |
| 48562 | 40145261 | 40145995 | |
| 48563 | 40161469 | 40162682 | |
| 48564 | 40172772 | 40175441 | 175600, 168303 |
| 48565 | 40409036 | 40410466 | 175600 |
| 48566 | 40411430 | 40412743 | 175600 |
| 48567 | 40442375 | 40444103 | 175600 |
| 48568 | 40453965 | 40455246 | 175600 |
| 48569 | 40475752 | 40478733 | 175600 |
| 48570 | 40508751 | 40511064 | 175600 |
| 48571 | 40794762 | 40795450 | 175600 |
| 48572 | 40799639 | 40801281 | 175600 |
| 48573 | 41025154 | 41027234 | |
| 48574 | 41042359 | 41048086 | |
| 48575 | 41094524 | 41095175 | |
| 48576 | 41142678 | 41143707 | 224017 |
| 48577 | 41146295 | 41149098 | 224017 |
| 48578 | 41149272 | 41150496 | 224017 |
| 48579 | 41355904 | 41356490 | |
| 48580 | 41454345 | 41456257 | |
| 48581 | 41476579 | 41478227 | |
| 48582 | 41486811 | 41489861 | |
| 48583 | 41498947 | 41500166 | |
| 48584 | 41503456 | 41504211 | |
| 48585 | 41504512 | 41507028 | |
| 48586 | 41548172 | 41549057 | |
| 48587 | 41730419 | 41732207 | 236310, 122641 |
| 48588 | 41732305 | 41733701 | 224116, 236310, 122641 |
| 48589 | 41734565 | 41737105 | 224116, 236310, 122641 |
| 48590 | 41742378 | 41743034 | 122641, 224116, 236310 |
| 48591 | 41743553 | 41750351 | 236310, 224116 |
| 48592 | 41750840 | 41751837 | 224116 |
| 48593 | 41981729 | 41983542 | |
| 48594 | 42896592 | 42896987 | |
| 48595 | 42927239 | 42929364 | |
| 48596 | 42950634 | 42952189 | 136197, 256646 |
| 48597 | 42970285 | 42973055 | 106591, 256646, 106588 |
| 48598 | 43522214 | 43522993 | 002746 |
| 48599 | 43526246 | 43527547 | 002746 |
| 48600 | 43528235 | 43530105 | 002746 |
| 48601 | 43541506 | 43541972 | 002746 |
| 48602 | 43622138 | 43624424 | 164543 |
| 48603 | 43679149 | 43680117 | 106603 |
| 48604 | 43686494 | 43688764 | 106603 |
| 48605 | 43697955 | 43700309 | 106603 |
| 48606 | 43713246 | 43715128 | 106603 |
| 48607 | 43732568 | 43734279 | 106603 |
| 48608 | 43768298 | 43770076 | 106603 |
| 48609 | 43804027 | 43804967 | 106605 |
| 48610 | 43821549 | 43822484 | 106605 |
| 48611 | 43873736 | 43874932 | |
| 48612 | 43878342 | 43878758 | |
| 48613 | 43908227 | 43909817 | 062582, 270617 |
| 48614 | 43912358 | 43913436 | 270617 |
| 48615 | 43943257 | 43946838 | 270617, 106608 |
| 48616 | 43965318 | 43966960 | 078967, 106608 |
| 48617 | 43973538 | 43974045 | 078967 |
| 48618 | 43977894 | 43978975 | 078967 |
| 48619 | 44083313 | 44085407 | 136279 |
| 48620 | 44115469 | 44116314 | 122678 |
| 48621 | 44121390 | 44122805 | 122678 |
| 48622 | 44161881 | 44163670 | 106628 |
| 48623 | 44196781 | 44197468 | 106633 |
| 48624 | 44201584 | 44202289 | 106633 |
| 48625 | 44240222 | 44241684 | 106636 |
| 48626 | 44362217 | 44362829 | 058404 |
| 48627 | 44512327 | 44513356 | 015676 |
| 48628 | 44516732 | 44517454 | 015676 |
| 48629 | 44529290 | 44531088 | 015676 |
| 48630 | 44577567 | 44578683 | 015520 |
| 48631 | 44578974 | 44584420 | 015520 |
| 48632 | 44587102 | 44588103 | |
| 48633 | 44601503 | 44602526 | |
| 48634 | 44602836 | 44603461 | |
| 48635 | 44612350 | 44614338 | 136271 |
| 48636 | 44620776 | 44622883 | 158604 |
| 48637 | 44645294 | 44647010 | 105953 |
| 48638 | 44661889 | 44662818 | 105953 |
| 48639 | 44673132 | 44674665 | 105953 |
| 48640 | 44677723 | 44679996 | 105953 |
| 48641 | 44717501 | 44719360 | 105953 |
| 48642 | 44787231 | 44789585 | 122515 |
| 48643 | 44804109 | 44804985 | 122515 |
| 48644 | 44809296 | 44809918 | 122515 |
| 48645 | 44835079 | 44837870 | 196262 |
| 48646 | 44844426 | 44845090 | 196262 |
| 48647 | 44850552 | 44851271 | 196262 |
| 48648 | 44886709 | 44888739 | 234183, 105968 |
| 48649 | 44905955 | 44907733 | |
| 48650 | 44923685 | 44926391 | 272768, 146676 |
| 48651 | 44933255 | 44933928 | |
| 48652 | 45025339 | 45027013 | 232956 |
| 48653 | 45038764 | 45040776 | 136280 |
| 48654 | 45054862 | 45055395 | 136280 |
| 48655 | 45150130 | 45152050 | 136270 |
| 48656 | 45728350 | 45730587 | 164742 |
| 48657 | 45730639 | 45731373 | 164742 |
| 48658 | 45734514 | 45735525 | 164742 |
| 48659 | 45741100 | 45742126 | 164742 |
| 48660 | 45919904 | 45923573 | |
| 48661 | 45923673 | 45925892 | |
| 48662 | 45926219 | 45929298 | 146678 |
| 48663 | 45960584 | 45961227 | 146674 |
| 48664 | 45961680 | 45964680 | |
| 48665 | 46016872 | 46019059 | 237471 |
| 48666 | 46063866 | 46064576 | |
| 48667 | 46177109 | 46179184 | |
| 48668 | 46182983 | 46186748 | 224547 |
| 48669 | 46191892 | 46196188 | |
| 48670 | 46320123 | 46322817 | 229459 |
| 48671 | 46531993 | 46532542 | |
| 48672 | 46563816 | 46566357 | |
| 48673 | 46583241 | 46586778 | |
| 48674 | 46588889 | 46590634 | |
| 48675 | 46694042 | 46695274 | |
| 48676 | 46719075 | 46720076 | 233539 |
| 48677 | 46734424 | 46735485 | 233539 |
| 48678 | 46744738 | 46745722 | 233539 |
| 48679 | 46753147 | 46754802 | 233539 |
| 48680 | 46755035 | 46755727 | 233539 |
| 48681 | 46802868 | 46804713 | |
| 48682 | 46971786 | 46973283 | 232072 |
| 48683 | 47186794 | 47187329 | |
| 48684 | 47221430 | 47221984 | |
| 48685 | 47293312 | 47294423 | |
| 48686 | 47305686 | 47306976 | |
| 48687 | 47322193 | 47323351 | 136205 |
| 48688 | 47445007 | 47446291 | 136205 |
| 48689 | 47485888 | 47487144 | 136205 |
| 48690 | 47491119 | 47495785 | 136205 |
| 48691 | 47496754 | 47498782 | 136205 |
| 48692 | 47500678 | 47501469 | 136205 |
| 48693 | 47509683 | 47511348 | 136205 |
| 48694 | 47513649 | 47515655 | 136205 |
| 48695 | 47520456 | 47522754 | 136205 |
| 48696 | 47524536 | 47525792 | 136205 |
| 48697 | 47536666 | 47539590 | 136205 |
| 48698 | 47539624 | 47540142 | 136205 |
| 48699 | 47542781 | 47543936 | 136205 |
| 48700 | 47547032 | 47550435 | 136205 |
| 48701 | 47550832 | 47554597 | 136205 |
| 48702 | 47556502 | 47561690 | 136205 |
| 48703 | 47569450 | 47570123 | 136205 |
| 48704 | 47575874 | 47577576 | 136205 |
| 48705 | 47578228 | 47579599 | 136205 |
| 48706 | 47610195 | 47614790 | 136205 |
| 48707 | 47615381 | 47616433 | 136205 |
| 48708 | 47619531 | 47622283 | 136205 |
| 48709 | 47639318 | 47642913 | |
| 48710 | 47643460 | 47647190 | |
| 48711 | 47649392 | 47651214 | |
| 48712 | 47674813 | 47675715 | |
| 48713 | 47730627 | 47731091 | |
| 48714 | 47801673 | 47803990 | 146666, 136273 |
| 48715 | 47822911 | 47824508 | 158683, 136273 |
| 48716 | 47825827 | 47826880 | 158683, 136273 |
| 48717 | 47844474 | 47845904 | 158683, 136275, 136273 |
| 48718 | 47885488 | 47886790 | 158683, 136273 |
| 48719 | 48018247 | 48019613 | 136273 |
| 48720 | 48031140 | 48032007 | 164744 |
| 48721 | 48123843 | 48129821 | 183696 |
| 48722 | 48134535 | 48135137 | 183696 |
| 48723 | 48149635 | 48150325 | |
| 48724 | 48179428 | 48180530 | |
| 48725 | 48183636 | 48184508 | |
| 48726 | 50517370 | 50518963 | 132436 |
| 48727 | 50523906 | 50524599 | |
| 48728 | 50541752 | 50543487 | 132437 |
| 48729 | 50569393 | 50571373 | 132437 |
| 48730 | 50598039 | 50600461 | 226122, 132437 |
| 48731 | 50601995 | 50603500 | 132437, 226122 |
| 48732 | 50623874 | 50624632 | 132437 |
| 48733 | 50628373 | 50629100 | 132437 |
| 48734 | 50631790 | 50633655 | 132437 |
| 48735 | 50682293 | 50683542 | 106070 |
| 48736 | 50700435 | 50701133 | 106070 |
| 48737 | 50724952 | 50725703 | 106070 |
| 48738 | 50734879 | 50736097 | 106070 |
| 48739 | 50744546 | 50745823 | 106070 |
| 48740 | 50749607 | 50750390 | 106070 |
| 48741 | 50751632 | 50752605 | 106070 |
| 48742 | 50764346 | 50765266 | 106070 |
| 48743 | 50838828 | 50839657 | 106070 |
| 48744 | 50845662 | 50846549 | 106070 |
| 48745 | 50859209 | 50862114 | 106070 |
| 48746 | 50880407 | 50881069 | |
| 48747 | 50933051 | 50933609 | |
| 48748 | 51190528 | 51191757 | 106078 |
| 48749 | 51207420 | 51208008 | 106078 |
| 48750 | 51292967 | 51295721 | 106078 |
| 48751 | 51316790 | 51317825 | 106078 |
| 48752 | 51318934 | 51319560 | 106078 |
| 48753 | 51331134 | 51331815 | 106078 |
| 48754 | 51332909 | 51334023 | 106078 |
| 48755 | 51382369 | 51385806 | 106078 |
| 48756 | 51386733 | 51387665 | |
| 48757 | 51391724 | 51394107 | |
| 48758 | 51395800 | 51397063 | |
| 48759 | 51407780 | 51408552 | |
| 48760 | 51411938 | 51413610 | |
| 48761 | 51424421 | 51425948 | |
| 48762 | 52085679 | 52086515 | |
| 48763 | 52214394 | 52215177 | |
| 48764 | 53486489 | 53487287 | |
| 48765 | 54825720 | 54828240 | 234707, 132432 |
| 48766 | 54856190 | 54857242 | 234707 |
| 48767 | 54907177 | 54907855 | |
| 48768 | 54946867 | 54949231 | |
| 48769 | 54955840 | 54956902 | |
| 48770 | 54971539 | 54972851 | |
| 48771 | 54975967 | 54976976 | |
| 48772 | 54985070 | 54986264 | |
| 48773 | 55000349 | 55001486 | 218586 |
| 48774 | 55034820 | 55036185 | |
| 48775 | 55055259 | 55056567 | |
| 48776 | 55085826 | 55090796 | 146648 |
| 48777 | 55098055 | 55102126 | 146648 |
| 48778 | 55115454 | 55116875 | 146648 |
| 48779 | 55123398 | 55125814 | 146648 |
| 48780 | 55131687 | 55135140 | 146648 |
| 48781 | 55140614 | 55143730 | 146648 |
| 48782 | 55144911 | 55146019 | 146648 |
| 48783 | 55159268 | 55160108 | 146648 |
| 48784 | 55166614 | 55167505 | 146648 |
| 48785 | 55175593 | 55176256 | 146648 |
| 48786 | 55186139 | 55190132 | 146648 |
| 48787 | 55196902 | 55203132 | 146648 |
| 48788 | 55207929 | 55209164 | 146648 |
| 48789 | 55246035 | 55247055 | 146648 |
| 48790 | 55321806 | 55323941 | 146648 |
| 48791 | 55432621 | 55435236 | 132434 |
| 48792 | 55479210 | 55480057 | 132434 |
| 48793 | 55482816 | 55483305 | 132434 |
| 48794 | 55600261 | 55603155 | 154978 |
| 48795 | 55613605 | 55615419 | 154978 |
| 48796 | 55638486 | 55641270 | 223475, 154978 |
| 48797 | 55641346 | 55642638 | 223475 |
| 48798 | 55661260 | 55661896 | 231394 |
| 48799 | 55954720 | 55955653 | 178665, 249773 |
| 48800 | 56019309 | 56020316 | 239789, 146729, 249773 |
| 48801 | 56031825 | 56033296 | 146729 |
| 48802 | 56064628 | 56065174 | 146729 |
| 48803 | 56067611 | 56068264 | 146729 |
| 48804 | 56074205 | 56075207 | |
| 48805 | 56096754 | 56097760 | 146733 |
| 48806 | 56101263 | 56102216 | 146733 |
| 48807 | 56118130 | 56120478 | 146731, 146733 |
| 48808 | 56131163 | 56133154 | 129103, 146731 |
| 48809 | 56141748 | 56142517 | 129103 |
| 48810 | 56147072 | 56148053 | 129103 |
| 48811 | 56173210 | 56174903 | 106153 |
| 48812 | 57410498 | 57411426 | |
| 48813 | 57411465 | 57412514 | |
| 48814 | 57437606 | 57439026 | |
| 48815 | 57927486 | 57929063 | |
| 48816 | 61968767 | 61969587 | |
| 48817 | 63360698 | 63361422 | 226581, 236299 |
| 48818 | 63385439 | 63387480 | 265017, 227910 |
| 48819 | 63767431 | 63768681 | 234444 |
| 48820 | 63774126 | 63775173 | 234444 |
| 48821 | 64022587 | 64024025 | 173041 |
| 48822 | 64059382 | 64060257 | |
| 48823 | 64088097 | 64089473 | |
| 48824 | 64804133 | 64804868 | |
| 48825 | 64838597 | 64839409 | 146757 |
| 48826 | 65215440 | 65216886 | 228409 |
| 48827 | 65337687 | 65338599 | 196715 |
| 48828 | 65372393 | 65373255 | 196715 |
| 48829 | 65389981 | 65390377 | 196715 |
| 48830 | 65411887 | 65412283 | 196715 |
| 48831 | 65417055 | 65417688 | 196715 |
| 48832 | 65420481 | 65421332 | 196715 |
| 48833 | 65425538 | 65426697 | 169919 |
| 48834 | 65426986 | 65427515 | 169919 |
| 48835 | 65446392 | 65447961 | 169919 |
| 48836 | 65508544 | 65509736 | |
| 48837 | 65515833 | 65516452 | |
| 48838 | 65540539 | 65542501 | 126522 |
| 48839 | 65564500 | 65565903 | 249319 |
| 48840 | 65578646 | 65581051 | 241258, 249319 |
| 48841 | 65590175 | 65591547 | 249319, 241258, 234185 |
| 48842 | 65606589 | 65609559 | 249319, 241258, 234185 |
| 48843 | 65610700 | 65614869 | 249319, 241258, 234185 |
| 48844 | 65615163 | 65616671 | 249319, 241258, 234185 |
| 48845 | 65617530 | 65618491 | 241258, 234185 |
| 48846 | 65627411 | 65628915 | 234185 |
| 48847 | 65648149 | 65650292 | |
| 48848 | 65652313 | 65653471 | |
| 48849 | 65654753 | 65656023 | |
| 48850 | 65669647 | 65671667 | 169902 |
| 48851 | 65688984 | 65689810 | 169902 |
| 48852 | 65711510 | 65713637 | 169902 |
| 48853 | 65732137 | 65732872 | 169902 |
| 48854 | 65747718 | 65749574 | 169902 |
| 48855 | 65761266 | 65762632 | 169902 |
| 48856 | 65772262 | 65773867 | 169902 |
| 48857 | 65957873 | 65959413 | 237310, 223473 |
| 48858 | 66000379 | 66001662 | 229180, 234500 |
| 48859 | 66056333 | 66057808 | 232559 |
| 48860 | 66092675 | 66094881 | 243335 |
| 48861 | 66118628 | 66120536 | 226824, 243335 |
| 48862 | 66146338 | 66147932 | 154710, 243335 |
| 48863 | 66204899 | 66206631 | 272831, 154710, 243335 |
| 48864 | 66309151 | 66310812 | 230583 |
| 48865 | 66385637 | 66387196 | 106609 |
| 48866 | 66459739 | 66460915 | 198874, 126524 |
| 48867 | 66767032 | 66768448 | 106610, 067601 |
| 48868 | 67157039 | 67157929 | 233423 |
| 48869 | 67203889 | 67204666 | |
| 48870 | 67229294 | 67229888 | |
| 48871 | 67319455 | 67320430 | |
| 48872 | 67641965 | 67642495 | 226829 |
| 48873 | 68713090 | 68714050 | 225718 |
| 48874 | 68715891 | 68716400 | 225718 |
| 48875 | 68807136 | 68807883 | 225718 |
| 48876 | 68877430 | 68878064 | 225718 |
| 48877 | 69061794 | 69064573 | 158321, 234215 |
| 48878 | 69265052 | 69265882 | 158321 |
| 48879 | 69335841 | 69337875 | 158321 |
| 48880 | 69839795 | 69840776 | 158321 |
| 48881 | 69927934 | 69928987 | 158321 |
| 48882 | 69939601 | 69943141 | 158321 |
| 48883 | 69984923 | 69986634 | 158321 |
| 48884 | 70033277 | 70037965 | 158321 |
| 48885 | 70060726 | 70061829 | 158321 |
| 48886 | 70061841 | 70062763 | 158321 |
| 48887 | 70063897 | 70064585 | 158321 |
| 48888 | 70067013 | 70068126 | 158321 |
| 48889 | 70079439 | 70080087 | 158321 |
| 48890 | 70133345 | 70134244 | 158321 |
| 48891 | 70146454 | 70148044 | 158321 |
| 48892 | 70158487 | 70161863 | 158321 |
| 48893 | 70177862 | 70178424 | 158321 |
| 48894 | 70186166 | 70187005 | 158321 |
| 48895 | 70246362 | 70246944 | 158321 |
| 48896 | 72385301 | 72386080 | 196313 |
| 48897 | 72394777 | 72396474 | 272843, 196313 |
| 48898 | 72722178 | 72723169 | 130305 |
| 48899 | 72771858 | 72772785 | 077800 |
| 48900 | 72935243 | 72937323 | 009954 |
| 48901 | 72970939 | 72972926 | 106635 |
| 48902 | 72973753 | 72975314 | |
| 48903 | 72991886 | 72993337 | 106638 |
| 48904 | 73001895 | 73002774 | |
| 48905 | 73026856 | 73029180 | 009950 |
| 48906 | 73036572 | 73039025 | 009950 |
| 48907 | 73050666 | 73051107 | |
| 48908 | 73061477 | 73063179 | |
| 48909 | 73078575 | 73080848 | |
| 48910 | 73081601 | 73082981 | 176428 |
| 48911 | 73096529 | 73098760 | 071462, 176410 |
| 48912 | 73135714 | 73136489 | |
| 48913 | 73152125 | 73154209 | 106077 |
| 48914 | 73156509 | 73157839 | |
| 48915 | 73184098 | 73185130 | 165215 |
| 48916 | 73188502 | 73189009 | |
| 48917 | 73256066 | 73257386 | 165171 |
| 48918 | 73265001 | 73266635 | |
| 48919 | 73269658 | 73270571 | |
| 48920 | 73284280 | 73286322 | |
| 48921 | 73294337 | 73295283 | |
| 48922 | 73393244 | 73393862 | |
| 48923 | 73475901 | 73476934 | 232415, 049540 |
| 48924 | 73587754 | 73589897 | 106682 |
| 48925 | 73634591 | 73636213 | 086730 |
| 48926 | 73654682 | 73655299 | 049541 |
| 48927 | 73668129 | 73669572 | 049541 |
| 48928 | 73683970 | 73684574 | |
| 48929 | 73693793 | 73694596 | |
| 48930 | 73699005 | 73699678 | |
| 48931 | 73703378 | 73704374 | 106665 |
| 48932 | 73794444 | 73795603 | 106665 |
| 48933 | 73867148 | 73870150 | 006704 |
| 48934 | 73891417 | 73896283 | 006704 |
| 48935 | 73896444 | 73897591 | 006704 |
| 48936 | 73897855 | 73898862 | 006704 |
| 48937 | 73915130 | 73916190 | 006704 |
| 48938 | 73983037 | 73983694 | 006704 |
| 48939 | 73993936 | 73996075 | 006704 |
| 48940 | 74020129 | 74021742 | 273069 |
| 48941 | 74024559 | 74025121 | |
| 48942 | 74041774 | 74042833 | |
| 48943 | 74070154 | 74073421 | 077809 |
| 48944 | 74074126 | 74075732 | 077809 |
| 48945 | 74106555 | 74107021 | 232729, 077809 |
| 48946 | 74488551 | 74489924 | 174374 |
| 48947 | 75156743 | 75157856 | 127957 |
| 48948 | 75240551 | 75241676 | 127946 |
| 48949 | 75267511 | 75269017 | 127946 |
| 48950 | 75278707 | 75279754 | 127946 |
| 48951 | 75305193 | 75305842 | 127946 |
| 48952 | 75321115 | 75321831 | 127946 |
| 48953 | 75367650 | 75369387 | 127946 |
| 48954 | 75471974 | 75472662 | 005486 |
| 48955 | 75507826 | 75509466 | 005486 |
| 48956 | 75534942 | 75537538 | 127948 |
| 48957 | 75543249 | 75552946 | 265020, 235090, 127948 |
| 48958 | 75554592 | 75557166 | 127948 |
| 48959 | 75566794 | 75568881 | 127948 |
| 48960 | 75572603 | 75575818 | 201643, 127948 |
| 48961 | 75580754 | 75581310 | 127948 |
| 48962 | 75585241 | 75586345 | 127948 |
| 48963 | 75587660 | 75589164 | 127948 |
| 48964 | 75609101 | 75610583 | 127948 |
| 48965 | 75618394 | 75619698 | 189077 |
| 48966 | 75620342 | 75620716 | 189077 |
| 48967 | 75622460 | 75624923 | 189077 |
| 48968 | 75666501 | 75667276 | 127952 |
| 48969 | 75676319 | 75678757 | 146701, 127952 |
| 48970 | 75686240 | 75687597 | 146701 |
| 48971 | 75698511 | 75698882 | |
| 48972 | 75735435 | 75737206 | 227038 |
| 48973 | 75795830 | 75796678 | |
| 48974 | 75807306 | 75807998 | |
| 48975 | 75920221 | 75921988 | |
| 48976 | 75923479 | 75924629 | |
| 48977 | 75929454 | 75930035 | |
| 48978 | 75931227 | 75935519 | 106211 |
| 48979 | 75935583 | 75937256 | |
| 48980 | 75939585 | 75940491 | |
| 48981 | 75946501 | 75948488 | |
| 48982 | 75962978 | 75963557 | 170027 |
| 48983 | 75986737 | 75989455 | 170027 |
| 48984 | 76008585 | 76010370 | |
| 48985 | 76021771 | 76023692 | 146700 |
| 48986 | 76637225 | 76637689 | 186704, 265479, 243566 |
| 48987 | 76873701 | 76875731 | 250990, 135205 |
| 48988 | 76945388 | 76946325 | 135205, 186088 |
| 48989 | 77012487 | 77013452 | 186088 |
| 48990 | 77021824 | 77022899 | 186088 |
| 48991 | 77037863 | 77041016 | 186088 |
| 48992 | 77044315 | 77046513 | 273341, 186088 |
| 48993 | 77165373 | 77168406 | 127947 |
| 48994 | 77314427 | 77315042 | 214293 |
| 48995 | 77320897 | 77322310 | 214293 |
| 48996 | 77324877 | 77327165 | 187257, 214293 |
| 48997 | 77395026 | 77396476 | 187257 |
| 48998 | 77426842 | 77428976 | 006576, 135211 |
| 48999 | 77647876 | 77650083 | 187391 |
| 49000 | 77755063 | 77755712 | 187391 |
| 49001 | 78066481 | 78067056 | 187391 |
| 49002 | 78840479 | 78841455 | 187391 |
| 49003 | 78937318 | 78938039 | 187391 |
| 49004 | 78946769 | 78947638 | 187391 |
| 49005 | 79049666 | 79050555 | 187391 |
| 49006 | 79063464 | 79064645 | 187391 |
| 49007 | 79080969 | 79084219 | 234456, 187391 |
| 49008 | 79090807 | 79091447 | 234456 |
| 49009 | 79763070 | 79766324 | 127955 |
| 49010 | 79776911 | 79777663 | 127955 |
| 49011 | 79779939 | 79781165 | 127955 |
| 49012 | 79839678 | 79840233 | 127955 |
| 49013 | 79847928 | 79848498 | 127955 |
| 49014 | 79851161 | 79851702 | |
| 49015 | 80014517 | 80015766 | 135218 |
| 49016 | 80078898 | 80080692 | 135218 |
| 49017 | 80240841 | 80241646 | 135218 |
| 49018 | 80263810 | 80264196 | 135218 |
| 49019 | 80547874 | 80548810 | 075223 |
| 49020 | 80570594 | 80572232 | |
| 49021 | 81232998 | 81235314 | 233491 |
| 49022 | 83097254 | 83098097 | 170381 |
| 49023 | 85158306 | 85158953 | |
| 49024 | 85183474 | 85184363 | |
| 49025 | 86475294 | 86476803 | 198822 |
| 49026 | 86780809 | 86783126 | 135164, 224046 |
| 49027 | 86848577 | 86849896 | 135185 |
| 49028 | 86865939 | 86866653 | |
| 49029 | 86940013 | 86940661 | |
| 49030 | 86973593 | 86975704 | 005469, 182165 |
| 49031 | 87042525 | 87045542 | 005471 |
| 49032 | 87092958 | 87093863 | 005471 |
| 49033 | 87102139 | 87105869 | 005471 |
| 49034 | 87108979 | 87112014 | 005471 |
| 49035 | 87124588 | 87126329 | |
| 49036 | 87126579 | 87127438 | |
| 49037 | 87157845 | 87158971 | 085563 |
| 49038 | 87177772 | 87178332 | 085563 |
| 49039 | 87193290 | 87194830 | 085563 |
| 49040 | 87197553 | 87201555 | 085563 |
| 49041 | 87202744 | 87205783 | 085563 |
| 49042 | 87228383 | 87230767 | 085563 |
| 49043 | 87237545 | 87238660 | 085563 |
| 49044 | 87257139 | 87258874 | 085563, 105784 |
| 49045 | 87504819 | 87506670 | 006634, 075303 |
| 49046 | 87674493 | 87675646 | 008277 |
| 49047 | 87848562 | 87850211 | 228113, 075142 |
| 49048 | 87855527 | 87857132 | 075142, 228113 |
| 49049 | 87918762 | 87919372 | 127954, 228113 |
| 49050 | 87940686 | 87941993 | |
| 49051 | 87968399 | 87969092 | |
| 49052 | 87987179 | 87988239 | |
| 49053 | 88435560 | 88436312 | 182348 |
| 49054 | 89792707 | 89793423 | 227646, 164647 |
| 49055 | 89802142 | 89803114 | 227646, 157214 |
| 49056 | 89807375 | 89810288 | 227646, 157214 |
| 49057 | 89824101 | 89825291 | 227646, 157214 |
| 49058 | 89840071 | 89841784 | 227646, 157214 |
| 49059 | 89845484 | 89846591 | 157214 |
| 49060 | 89873947 | 89875136 | 105792 |
| 49061 | 89895727 | 89897506 | 234459, 105792 |
| 49062 | 89900860 | 89901453 | 105792 |
| 49063 | 89974604 | 89977103 | 105793 |
| 49064 | 90031957 | 90035409 | 273299, 157224 |
| 49065 | 90038704 | 90039733 | 273299, 157224 |
| 49066 | 90040189 | 90041868 | 273299, 157224 |
| 49067 | 90193200 | 90194547 | 058091 |
| 49068 | 90221318 | 90222112 | 058091, 223969 |
| 49069 | 90224159 | 90226922 | 223969, 058091 |
| 49070 | 90254518 | 90255909 | 058091 |
| 49071 | 90346607 | 90347555 | 058091 |
| 49072 | 90350064 | 90350991 | 058091 |
| 49073 | 90395545 | 90398098 | 058091 |
| 49074 | 90445434 | 90446763 | 058091 |
| 49075 | 90523457 | 90523791 | 058091 |
| 49076 | 90893444 | 90894497 | 157240 |
| 49077 | 91479724 | 91480174 | 127989 |
| 49078 | 91509151 | 91510700 | 127989 |
| 49079 | 91569374 | 91571045 | 127914 |
| 49080 | 91653900 | 91655703 | 127914 |
| 49081 | 91671005 | 91673912 | 127914 |
| 49082 | 91705803 | 91706357 | 127914 |
| 49083 | 91762686 | 91765156 | 188693, 001630, 240720 |
| 49084 | 91808154 | 91808804 | 188693, 240720 |
| 49085 | 91874352 | 91876371 | 001629, 001631 |
| 49086 | 92053492 | 92054314 | 230927 |
| 49087 | 92076300 | 92078662 | 157259 |
| 49088 | 92156731 | 92158858 | 127993, 127980 |
| 49089 | 92218664 | 92219942 | 234545 |
| 49090 | 92261847 | 92265694 | 105810 |
| 49091 | 92284639 | 92285275 | 105810 |
| 49092 | 92324521 | 92325813 | 105810 |
| 49093 | 92380856 | 92382884 | 105810 |
| 49094 | 92392684 | 92393445 | 105810 |
| 49095 | 92394449 | 92398533 | 105810 |
| 49096 | 92410315 | 92411194 | 105810 |
| 49097 | 92426698 | 92427488 | 105810 |
| 49098 | 92438226 | 92439590 | 105810 |
| 49099 | 92460920 | 92466486 | 237819, 105810 |
| 49100 | 92860759 | 92862442 | 004766 |
| 49101 | 93207807 | 93208572 | |
| 49102 | 93609982 | 93610994 | 236861, 105829 |
| 49103 | 93632463 | 93634012 | 105829, 236861 |
| 49104 | 93930156 | 93931095 | |
| 49105 | 94138349 | 94140410 | 127995 |
| 49106 | 94208880 | 94210924 | |
| 49107 | 94213222 | 94214410 | |
| 49108 | 94223957 | 94224880 | 127990 |
| 49109 | 94249948 | 94251137 | 127990 |
| 49110 | 94284382 | 94286339 | 242265, 127990 |
| 49111 | 94325905 | 94327990 | |
| 49112 | 94536530 | 94538002 | 158528 |
| 49113 | 94696275 | 94696977 | 158528 |
| 49114 | 94870125 | 94870679 | 158528 |
| 49115 | 94946901 | 94951137 | 005421 |
| 49116 | 94951703 | 94954533 | 005421 |
| 49117 | 94964274 | 94965062 | 005421 |
| 49118 | 95024746 | 95026738 | 105852, 005421 |
| 49119 | 95060072 | 95060665 | 105854 |
| 49120 | 95063247 | 95065215 | 105854 |
| 49121 | 95068987 | 95071353 | |
| 49122 | 95174209 | 95175376 | |
| 49123 | 95175860 | 95176466 | |
| 49124 | 95178627 | 95180448 | |
| 49125 | 95189310 | 95190570 | |
| 49126 | 95224415 | 95226413 | 231170, 004799 |
| 49127 | 95237375 | 95239468 | 231170 |
| 49128 | 95755723 | 95756221 | 004864 |
| 49129 | 95818394 | 95818980 | 004864 |
| 49130 | 95848653 | 95849165 | 208025, 004864 |
| 49131 | 95877444 | 95878348 | 004864 |
| 49132 | 95895167 | 95896983 | 004864 |
| 49133 | 95932185 | 95933153 | 004864 |
| 49134 | 95937440 | 95938461 | 004864 |
| 49135 | 95950008 | 95952194 | 004864 |
| 49136 | 95988616 | 95989752 | |
| 49137 | 96030365 | 96031015 | |
| 49138 | 96043887 | 96045490 | |
| 49139 | 96131909 | 96133349 | 197851, 127922 |
| 49140 | 96137279 | 96138047 | 127922 |
| 49141 | 96163156 | 96164362 | 127922 |
| 49142 | 96175735 | 96179257 | 127922 |
| 49143 | 96228538 | 96229485 | 127922 |
| 49144 | 96234335 | 96235102 | 127922 |
| 49145 | 96338190 | 96339774 | 127922 |
| 49146 | 96347792 | 96348279 | |
| 49147 | 96429350 | 96430681 | |
| 49148 | 96455382 | 96455836 | |
| 49149 | 96462910 | 96463559 | |
| 49150 | 96578966 | 96579620 | |
| 49151 | 96746440 | 96747883 | 196636 |
| 49152 | 97600801 | 97602347 | 243554 |
| 49153 | 97650748 | 97651753 | |
| 49154 | 97714719 | 97716852 | |
| 49155 | 97735027 | 97737450 | 164715 |
| 49156 | 97739841 | 97740671 | 164715 |
| 49157 | 97746243 | 97750696 | 164715 |
| 49158 | 97760057 | 97764612 | 164715 |
| 49159 | 97797758 | 97798381 | 164715 |
| 49160 | 97803534 | 97804184 | 164715 |
| 49161 | 97840465 | 97841668 | 180535 |
| 49162 | 97880937 | 97882411 | 164713, 205356 |
| 49163 | 97909895 | 97917071 | 164713 |
| 49164 | 97929811 | 97930823 | 006453, 164713 |
| 49165 | 97977518 | 97978749 | 006453 |
| 49166 | 97983178 | 97984482 | 006453 |
| 49167 | 97986546 | 97987798 | 006453 |
| 49168 | 97989629 | 97990425 | 006453 |
| 49169 | 98000153 | 98001349 | 006453 |
| 49170 | 98021476 | 98022153 | 006453 |
| 49171 | 98023681 | 98024174 | 006453 |
| 49172 | 98027801 | 98031086 | 006453 |
| 49173 | 98045882 | 98049933 | |
| 49174 | 98050213 | 98050759 | |
| 49175 | 98248999 | 98249691 | 106236 |
| 49176 | 98475655 | 98477625 | 196367 |
| 49177 | 98519555 | 98521313 | 196367 |
| 49178 | 98648094 | 98649463 | 198742 |
| 49179 | 98664870 | 98665376 | 198742 |
| 49180 | 98688735 | 98691012 | 198742 |
| 49181 | 98692910 | 98697590 | 198742 |
| 49182 | 98738602 | 98740599 | 198742 |
| 49183 | 98740604 | 98742533 | 198742 |
| 49184 | 98759905 | 98760486 | |
| 49185 | 98767691 | 98768979 | |
| 49186 | 98787981 | 98788778 | 185467 |
| 49187 | 98797693 | 98799868 | 185467 |
| 49188 | 98804291 | 98810812 | 185467 |
| 49189 | 98821375 | 98823921 | |
| 49190 | 98922680 | 98924567 | 241685 |
| 49191 | 98969965 | 98970866 | 241685 |
| 49192 | 98971691 | 98973599 | 130429, 241685 |
| 49193 | 98978227 | 98979092 | 241685, 130429 |
| 49194 | 98979163 | 98979784 | 241685, 130429 |
| 49195 | 99004977 | 99007638 | 106245, 106244 |
| 49196 | 99035771 | 99037325 | 160917, 248919, 106246 |
| 49197 | 99062545 | 99064471 | 241468, 248919, 106246 |
| 49198 | 99069299 | 99069793 | |
| 49199 | 99070043 | 99071190 | 198556 |
| 49200 | 99096901 | 99098176 | 160908, 198556 |
| 49201 | 99101885 | 99103642 | 196652 |
| 49202 | 99148743 | 99150039 | 221909 |
| 49203 | 99155066 | 99157419 | 197343, 272647, 221909 |
| 49204 | 99159052 | 99161429 | 197343, 272647 |
| 49205 | 99213745 | 99215339 | 197037 |
| 49206 | 99249023 | 99251842 | 106258 |
| 49207 | 99267336 | 99267798 | 106258 |
| 49208 | 99273494 | 99276415 | 106258 |
| 49209 | 99276534 | 99278236 | 106258 |
| 49210 | 99282894 | 99283457 | |
| 49211 | 99291036 | 99293214 | |
| 49212 | 99327450 | 99327997 | 160870 |
| 49213 | 99337234 | 99341414 | |
| 49214 | 99387534 | 99393679 | |
| 49215 | 99421612 | 99428402 | 021461 |
| 49216 | 99441445 | 99441880 | 021461 |
| 49217 | 99457662 | 99458716 | 021461 |
| 49218 | 99516073 | 99517934 | 146833 |
| 49219 | 99550406 | 99553807 | |
| 49220 | 99571494 | 99572037 | 160862 |
| 49221 | 99572042 | 99574919 | 160862 |
| 49222 | 99594624 | 99596161 | |
| 49223 | 99601140 | 99602471 | |
| 49224 | 99612927 | 99615469 | 106261 |
| 49225 | 99620297 | 99620702 | 106261 |
| 49226 | 99621361 | 99622146 | 106261 |
| 49227 | 99634153 | 99635309 | 106261 |
| 49228 | 99642283 | 99642792 | |
| 49229 | 99646590 | 99648533 | 166529 |
| 49230 | 99662880 | 99663808 | 166526 |
| 49231 | 99678395 | 99680747 | 166526 |
| 49232 | 99685853 | 99687451 | 168090 |
| 49233 | 99697561 | 99699795 | 221838, 166508 |
| 49234 | 99723727 | 99726385 | 214309, 242798 |
| 49235 | 99727888 | 99729079 | 235077 |
| 49236 | 99746274 | 99747305 | 188186 |
| 49237 | 99754857 | 99756648 | 146826 |
| 49238 | 100025232 | 100028524 | 146834, 078487 |
| 49239 | 100032621 | 100034477 | 160813, 240211, 241357 |
| 49240 | 100041560 | 100043123 | |
| 49241 | 100047769 | 100048787 | |
| 49242 | 100075868 | 100077406 | 166925 |
| 49243 | 100080562 | 100081595 | 166924, 266372 |
| 49244 | 100132354 | 100132870 | |
| 49245 | 100136193 | 100138431 | 106351 |
| 49246 | 100141500 | 100146176 | 106351 |
| 49247 | 100164960 | 100166399 | 106351 |
| 49248 | 100166859 | 100169562 | 184414 |
| 49249 | 100182566 | 100184647 | 077454, 106336 |
| 49250 | 100199670 | 100199974 | 106333, 224729 |
| 49251 | 100201601 | 100202630 | 224729, 106333 |
| 49252 | 100202882 | 100203670 | 106333 |
| 49253 | 100209442 | 100210859 | 106330 |
| 49254 | 100219133 | 100220062 | 106327 |
| 49255 | 100237976 | 100240400 | 106327 |
| 49256 | 100270678 | 100274493 | 172354 |
| 49257 | 100290766 | 100292800 | |
| 49258 | 100302813 | 100304931 | 172336 |
| 49259 | 100324901 | 100325529 | |
| 49260 | 100424288 | 100425866 | 146828, 196411 |
| 49261 | 100429092 | 100429852 | 146828 |
| 49262 | 100434392 | 100435465 | 236305, 146828 |
| 49263 | 100449378 | 100450706 | 236305, 146828 |
| 49264 | 100464172 | 100466703 | 087077, 146828 |
| 49265 | 100471669 | 100473584 | 087087 |
| 49266 | 100486227 | 100488934 | 176125, 087085, 087087 |
| 49267 | 100492153 | 100495511 | 087085 |
| 49268 | 100510097 | 100511148 | |
| 49269 | 100511458 | 100513490 | |
| 49270 | 100514009 | 100514474 | |
| 49271 | 100546687 | 100547883 | 169894, 228273 |
| 49272 | 100608846 | 100609854 | 169894, 225946 |
| 49273 | 100660317 | 100661101 | 227053, 205277 |
| 49274 | 100727723 | 100729848 | 169871 |
| 49275 | 100742169 | 100748981 | |
| 49276 | 100751510 | 100752743 | |
| 49277 | 100759640 | 100762399 | |
| 49278 | 100764305 | 100776491 | 106366 |
| 49279 | 100776580 | 100778350 | 106366 |
| 49280 | 100778507 | 100779492 | 106366 |
| 49281 | 100780178 | 100785240 | 106366 |
| 49282 | 100787105 | 100787977 | |
| 49283 | 100797501 | 100798826 | 106367 |
| 49284 | 100843774 | 100844772 | 106384 |
| 49285 | 100859450 | 100862442 | 106400, 106397 |
| 49286 | 100863367 | 100864277 | 106400 |
| 49287 | 100880859 | 100881391 | 106404 |
| 49288 | 100886908 | 100888662 | 214253 |
| 49289 | 100894832 | 100896081 | 214253 |
| 49290 | 100964407 | 100966117 | 128581 |
| 49291 | 100995472 | 100996369 | |
| 49292 | 101006219 | 101007667 | 160963 |
| 49293 | 101029781 | 101030461 | 160963 |
| 49294 | 101178621 | 101181125 | 160963 |
| 49295 | 101228134 | 101228626 | |
| 49296 | 101331164 | 101332205 | |
| 49297 | 101335030 | 101336089 | |
| 49298 | 101360852 | 101361337 | |
| 49299 | 101376713 | 101377580 | |
| 49300 | 101397690 | 101398307 | |
| 49301 | 101457326 | 101459869 | 257923 |
| 49302 | 101459873 | 101461630 | 257923 |
| 49303 | 101482013 | 101482560 | 257923 |
| 49304 | 101499674 | 101500708 | 257923 |
| 49305 | 101501998 | 101503777 | 257923 |
| 49306 | 101524823 | 101527302 | 257923 |
| 49307 | 101555306 | 101555978 | 257923 |
| 49308 | 101578444 | 101580804 | 257923 |
| 49309 | 101584645 | 101586444 | 257923 |
| 49310 | 101595506 | 101597480 | 257923 |
| 49311 | 101667526 | 101668783 | 257923 |
| 49312 | 101764706 | 101766233 | 257923 |
| 49313 | 101875713 | 101876564 | 257923 |
| 49314 | 101877011 | 101877971 | 257923 |
| 49315 | 101901119 | 101902151 | 257923 |
| 49316 | 101929427 | 101929948 | 160999 |
| 49317 | 102035908 | 102037914 | 128563 |
| 49318 | 102104581 | 102106150 | 161036, 160993 |
| 49319 | 102110149 | 102111370 | 161036 |
| 49320 | 102452373 | 102452938 | |
| 49321 | 102513759 | 102517537 | 161040 |
| 49322 | 102631594 | 102634370 | 161040 |
| 49323 | 102714466 | 102716575 | 170632, 161040 |
| 49324 | 102753600 | 102754019 | 161048 |
| 49325 | 102788659 | 102790796 | 161048 |
| 49326 | 102937472 | 102938759 | 105819 |
| 49327 | 102983941 | 102985939 | 161057, 105821 |
| 49328 | 102987481 | 102988824 | 161057 |
| 49329 | 103028980 | 103030090 | 170615 |
| 49330 | 103300170 | 103304697 | 189056 |
| 49331 | 103449896 | 103451230 | 189056 |
| 49332 | 103576962 | 103577483 | 189056 |
| 49333 | 103630328 | 103631055 | |
| 49334 | 103636758 | 103637934 | |
| 49335 | 103650306 | 103651401 | |
| 49336 | 103847716 | 103849245 | 164815 |
| 49337 | 104563239 | 104563924 | 225329 |
| 49338 | 104612046 | 104612632 | 228393 |
| 49339 | 104623221 | 104625793 | 228393 |
| 49340 | 104629371 | 104630980 | 228393 |
| 49341 | 104651598 | 104657038 | 005483, 239569, 228393 |
| 49342 | 104685247 | 104685663 | 005483 |
| 49343 | 104686848 | 104687343 | 005483 |
| 49344 | 104771876 | 104772849 | 135250 |
| 49345 | 104776661 | 104777021 | 135250 |
| 49346 | 104818957 | 104819982 | 135250 |
| 49347 | 105028345 | 105030062 | 135250 |
| 49348 | 105079542 | 105080528 | 091127 |
| 49349 | 105161388 | 105163191 | 091127 |
| 49350 | 105172264 | 105173643 | 135249 |
| 49351 | 105221585 | 105222360 | 185055 |
| 49352 | 105288088 | 105291016 | 146776 |
| 49353 | 105305254 | 105305685 | 146776 |
| 49354 | 105313614 | 105315645 | 146776 |
| 49355 | 105316134 | 105316877 | 146776 |
| 49356 | 105317091 | 105318142 | 146776 |
| 49357 | 105318440 | 105320365 | 146776 |
| 49358 | 105320969 | 105321458 | 146776 |
| 49359 | 105358708 | 105359741 | 146776 |
| 49360 | 105410320 | 105411182 | 146776 |
| 49361 | 105434859 | 105436615 | 146776 |
| 49362 | 105442784 | 105444121 | 146776 |
| 49363 | 105451007 | 105452788 | 146776 |
| 49364 | 105466553 | 105467274 | 146776 |
| 49365 | 105470042 | 105471873 | 146776 |
| 49366 | 105516034 | 105518760 | 128536, 146776 |
| 49367 | 105637529 | 105638095 | 128536 |
| 49368 | 105649702 | 105650420 | 128536 |
| 49369 | 105705086 | 105706756 | |
| 49370 | 105710273 | 105711174 | |
| 49371 | 105751380 | 105753615 | 008282 |
| 49372 | 105780098 | 105780804 | |
| 49373 | 105816913 | 105817667 | |
| 49374 | 105818692 | 105821427 | |
| 49375 | 105836649 | 105837608 | |
| 49376 | 105847184 | 105848110 | |
| 49377 | 105856131 | 105858586 | |
| 49378 | 105879718 | 105880431 | |
| 49379 | 105896488 | 105897206 | 105835 |
| 49380 | 105923548 | 105926953 | 273320, 105835 |
| 49381 | 105953299 | 105955058 | |
| 49382 | 105966715 | 105968992 | |
| 49383 | 105969544 | 105971452 | |
| 49384 | 106018152 | 106018987 | 243797 |
| 49385 | 106056879 | 106059660 | 243797 |
| 49386 | 106064305 | 106067222 | 243797 |
| 49387 | 106088824 | 106090479 | 243797 |
| 49388 | 106096406 | 106097370 | 243797 |
| 49389 | 106124788 | 106128063 | 243797 |
| 49390 | 106144586 | 106144986 | 243797 |
| 49391 | 106170659 | 106171018 | 243797 |
| 49392 | 106182489 | 106183815 | 243797 |
| 49393 | 106208316 | 106210218 | 243797 |
| 49394 | 106251158 | 106253336 | 243797 |
| 49395 | 106261157 | 106262746 | 243797 |
| 49396 | 106263040 | 106265465 | 243797 |
| 49397 | 106280038 | 106281819 | 243797 |
| 49398 | 106298700 | 106302295 | 177820, 253276, 243797 |
| 49399 | 106358319 | 106359146 | 243797 |
| 49400 | 106410596 | 106411221 | 243797 |
| 49401 | 106413070 | 106414279 | |
| 49402 | 106762744 | 106763402 | 005249, 243193 |
| 49403 | 106807493 | 106810787 | 105856 |
| 49404 | 107200363 | 107201005 | 164597 |
| 49405 | 107203430 | 107205700 | 105865, 164597 |
| 49406 | 107219768 | 107221625 | 272854, 075790 |
| 49407 | 107383121 | 107385484 | 105879, 241764 |
| 49408 | 107476697 | 107477229 | |
| 49409 | 107500132 | 107501106 | |
| 49410 | 107501281 | 107502215 | |
| 49411 | 107530792 | 107532806 | 091140 |
| 49412 | 107589196 | 107590293 | 091136 |
| 49413 | 107607120 | 107608372 | 091136 |
| 49414 | 107612162 | 107613665 | 091136 |
| 49415 | 107644710 | 107645285 | |
| 49416 | 107652682 | 107653700 | |
| 49417 | 107851669 | 107852694 | 091129 |
| 49418 | 107885357 | 107888055 | 091129 |
| 49419 | 107939564 | 107940170 | 091129 |
| 49420 | 107998790 | 107999732 | 266112, 091 129 |
| 49421 | 108165680 | 108167547 | 135241 |
| 49422 | 108208909 | 108212143 | 128590, 177683, 135241 |
| 49423 | 108248968 | 108249887 | 225647 |
| 49424 | 108249991 | 108250556 | 225647 |
| 49425 | 108253501 | 108255276 | 225647 |
| 49426 | 108293790 | 108294604 | |
| 49427 | 109724241 | 109725044 | |
| 49428 | 110162259 | 110163364 | 226965 |
| 49429 | 110174032 | 110175377 | 226965 |
| 49430 | 110748130 | 110749191 | 184903, 173114 |
| 49431 | 110753142 | 110754878 | 184903, 173114 |
| 49432 | 110832424 | 110833293 | 184903 |
| 49433 | 110872215 | 110873318 | 184903 |
| 49434 | 111020651 | 111021678 | 184903 |
| 49435 | 111064600 | 111065480 | 184903 |
| 49436 | 111138991 | 111139541 | 184903 |
| 49437 | 111156990 | 111158155 | 184903 |
| 49438 | 111177158 | 111177601 | 184903 |
| 49439 | 111201246 | 111202888 | 184903 |
| 49440 | 111405903 | 111406509 | 128512 |
| 49441 | 111443063 | 111443917 | 128512 |
| 49442 | 111593480 | 111594182 | 222346, 128512 |
| 49443 | 111649123 | 111649653 | 128512 |
| 49444 | 111653084 | 111653775 | 128512 |
| 49445 | 111692770 | 111693477 | 128512 |
| 49446 | 111695765 | 111697866 | 128512 |
| 49447 | 111719826 | 111721834 | 128512 |
| 49448 | 111726229 | 111727222 | 128512 |
| 49449 | 111752855 | 111753436 | 128512 |
| 49450 | 111769841 | 111770420 | 128512 |
| 49451 | 111802223 | 111803010 | 128512 |
| 49452 | 111833821 | 111834754 | 128512 |
| 49453 | 111838866 | 111840577 | 128512 |
| 49454 | 111843829 | 111847891 | 198839, 128512 |
| 49455 | 111851397 | 111852317 | 198839 |
| 49456 | 111863274 | 111863840 | 198839 |
| 49457 | 111872720 | 111873827 | 198839 |
| 49458 | 111878259 | 111879965 | 198839 |
| 49459 | 112031783 | 112032875 | 226851 |
| 49460 | 112089287 | 112092724 | 006652 |
| 49461 | 112093508 | 112094570 | 006652 |
| 49462 | 112102197 | 112102688 | 006652 |
| 49463 | 112429328 | 112430891 | 146802 |
| 49464 | 112579234 | 112580949 | 164603 |
| 49465 | 112758253 | 112759198 | 214194, 225457 |
| 49466 | 113721781 | 113728530 | 128573 |
| 49467 | 113749259 | 113750005 | 128573 |
| 49468 | 113798287 | 113798741 | 128573 |
| 49469 | 113822770 | 113823964 | 128573 |
| 49470 | 113882842 | 113884105 | 128573 |
| 49471 | 113895498 | 113896003 | 128573 |
| 49472 | 114045433 | 114045941 | 128573 |
| 49473 | 114054180 | 114057078 | 224595, 128573 |
| 49474 | 114561536 | 114563811 | 135272 |
| 49475 | 114619273 | 114620618 | 135272 |
| 49476 | 114645769 | 114646762 | 135272 |
| 49477 | 114653351 | 114654468 | 135272 |
| 49478 | 114695945 | 114696457 | |
| 49479 | 114726543 | 114727967 | 233607, 225535 |
| 49480 | 114728867 | 114729438 | 233607, 225535 |
| 49481 | 114739866 | 114740447 | 233607, 225535 |
| 49482 | 114750443 | 114751222 | 233607, 225535 |
| 49483 | 114753600 | 114754344 | 233607, 225535 |
| 49484 | 114785421 | 114786237 | 233607 |
| 49485 | 115318032 | 115318929 | |
| 49486 | 115809068 | 115809909 | |
| 49487 | 115850177 | 115852080 | 135269 |
| 49488 | 115858020 | 115858756 | 135269 |
| 49489 | 115879261 | 115880100 | 243243, 237813, 135269 |
| 49490 | 115901543 | 115902570 | 243243, 237813 |
| 49491 | 115910975 | 115914476 | 243243, 237813 |
| 49492 | 115923661 | 115924640 | 237870, 243243, 237813 |
| 49493 | 115955253 | 115958604 | 243243, 105971, 237813 |
| 49494 | 115976988 | 115981179 | 105971, 237813 |
| 49495 | 115993610 | 115997560 | 105971, 237813 |
| 49496 | 116034658 | 116035939 | 105971, 237813 |
| 49497 | 116066441 | 116067041 | 105971, 237813 |
| 49498 | 116081494 | 116084390 | 105971, 237813 |
| 49499 | 116104816 | 116107115 | 105971, 237813 |
| 49500 | 116138828 | 116143775 | 237813, 105971 |
| 49501 | 116150939 | 116153451 | |
| 49502 | 116164141 | 116167368 | 105974 |
| 49503 | 116176128 | 116176689 | 105974 |
| 49504 | 116268892 | 116271979 | |
| 49505 | 116272887 | 116274693 | |
| 49506 | 116292664 | 116293872 | |
| 49507 | 116310720 | 116315527 | 105976 |
| 49508 | 116319611 | 116320062 | 105976 |
| 49509 | 116326883 | 116329277 | 105976 |
| 49510 | 116345906 | 116347899 | 105976 |
| 49511 | 116350579 | 116351787 | 105976 |
| 49512 | 116356573 | 116357683 | 105976 |
| 49513 | 116377098 | 116377897 | 105976 |
| 49514 | 116381143 | 116382481 | 105976 |
| 49515 | 116411259 | 116414140 | 105976 |
| 49516 | 116415819 | 116418123 | 105976 |
| 49517 | 116420678 | 116421457 | 105976 |
| 49518 | 116442824 | 116443266 | |
| 49519 | 116444188 | 116446402 | |
| 49520 | 116449430 | 116453326 | 198898 |
| 49521 | 116502265 | 116503916 | 198898 |
| 49522 | 116592660 | 116595162 | 004866, 214188, 227199 |
| 49523 | 116655427 | 116656027 | 004866, 214188 |
| 49524 | 116687639 | 116688117 | 004866, 214188 |
| 49525 | 116716107 | 116717293 | 004866, 214188, 226367 |
| 49526 | 116764396 | 116765341 | 004866, 226367 |
| 49527 | 116792475 | 116793379 | 004866 |
| 49528 | 116797243 | 116798431 | 004866 |
| 49529 | 116809684 | 116811187 | 004866 |
| 49530 | 117129782 | 117130660 | 001626 |
| 49531 | 117207909 | 117208326 | 001626 |
| 49532 | 117221770 | 117222869 | 001626 |
| 49533 | 117245661 | 117246272 | 083622, 001626 |
| 49534 | 117512773 | 117513983 | 077063 |
| 49535 | 117577401 | 117578548 | |
| 49536 | 117823097 | 117825039 | 128534 |
| 49537 | 118981805 | 118982512 | |
| 49538 | 119154374 | 119154897 | |
| 49539 | 120382231 | 120384176 | 184408 |
| 49540 | 120496919 | 120498789 | 106025 |
| 49541 | 120590318 | 120592114 | 071243 |
| 49542 | 120627529 | 120631429 | 106034 |
| 49543 | 120644163 | 120644876 | 106034 |
| 49544 | 120709381 | 120709874 | 106034 |
| 49545 | 120719313 | 120720429 | 106034 |
| 49546 | 120722416 | 120726995 | 212628, 106034 |
| 49547 | 120777817 | 120780543 | 106034 |
| 49548 | 120797113 | 120798074 | 106034 |
| 49549 | 120945004 | 120945561 | |
| 49550 | 120983200 | 120984519 | |
| 49551 | 121034545 | 121035251 | 196937 |
| 49552 | 121035492 | 121037317 | 196937 |
| 49553 | 121037959 | 121038680 | 213310 |
| 49554 | 121354312 | 121355359 | |
| 49555 | 121783252 | 121785314 | 008311 |
| 49556 | 121838960 | 121839783 | |
| 49557 | 121857076 | 121857643 | 234418 |
| 49558 | 121944504 | 121945258 | 128610, 230316 |
| 49559 | 121956770 | 121957620 | |
| 49560 | 122155302 | 122155910 | 081803 |
| 49561 | 122525803 | 122527247 | 081803 |
| 49562 | 122830369 | 122832402 | 081800 |
| 49563 | 123174453 | 123175351 | 164675 |
| 49564 | 123196922 | 123198452 | 128609 |
| 49565 | 123238425 | 123239563 | 146809, 230442 |
| 49566 | 123262380 | 123263471 | 146809, 230442 |
| 49567 | 123283527 | 123284793 | |
| 49568 | 123309460 | 123310041 | |
| 49569 | 123314811 | 123315539 | |
| 49570 | 123319533 | 123320074 | |
| 49571 | 123320938 | 123322127 | 106299 |
| 49572 | 123387324 | 123390234 | 272686, 106299 |
| 49573 | 123409334 | 123409881 | |
| 49574 | 123410525 | 123410920 | |
| 49575 | 123437520 | 123438597 | |
| 49576 | 123516412 | 123517209 | 106302 |
| 49577 | 124129084 | 124131082 | |
| 49578 | 124175938 | 124176933 | |
| 49579 | 124357990 | 124358729 | |
| 49580 | 124360249 | 124360694 | |
| 49581 | 124361797 | 124363579 | |
| 49582 | 124364271 | 124365534 | |
| 49583 | 124402447 | 124403865 | 170775 |
| 49584 | 124403907 | 124405094 | 170775 |
| 49585 | 124405139 | 124406430 | 170775 |
| 49586 | 124569168 | 124570537 | 224897, 128513 |
| 49587 | 126214042 | 126215233 | 179603 |
| 49588 | 126297507 | 126298307 | 179603 |
| 49589 | 126577077 | 126578141 | 179603 |
| 49590 | 126580374 | 126581865 | 179603 |
| 49591 | 126675174 | 126675948 | 179603 |
| 49592 | 126681755 | 126682515 | 179603 |
| 49593 | 126891248 | 126894362 | 179603 |
| 49594 | 126987571 | 126988397 | 048405 |
| 49595 | 127031276 | 127034212 | 048405 |
| 49596 | 127224570 | 127226369 | 179562 |
| 49597 | 127227738 | 127229304 | 004059, 179562 |
| 49598 | 127233160 | 127234170 | 179562, 106328 |
| 49599 | 127234346 | 127235073 | 106328 |
| 49600 | 127290929 | 127293191 | 197157, 240790 |
| 49601 | 127347551 | 127348016 | 197157 |
| 49602 | 127399270 | 127400999 | 197157 |
| 49603 | 127547493 | 127548220 | 197157 |
| 49604 | 127640494 | 127641381 | 197157 |
| 49605 | 127645299 | 127646456 | 197157 |
| 49606 | 127775230 | 127776170 | |
| 49607 | 127950471 | 127950906 | 106344 |
| 49608 | 127983103 | 127984417 | 238750, 106344 |
| 49609 | 128023637 | 128024198 | |
| 49610 | 128094293 | 128096261 | 240758, 135245, 273184 |
| 49611 | 128170949 | 128171918 | 242588, 273270 |
| 49612 | 128378241 | 128380752 | 128595 |
| 49613 | 128399101 | 128400363 | 128595 |
| 49614 | 128469590 | 128470507 | 128591 |
| 49615 | 128502507 | 128504275 | 128524, 242902, 135253 |
| 49616 | 128524165 | 128524708 | 135253 |
| 49617 | 128577616 | 128579197 | 128604 |
| 49618 | 128602861 | 128603833 | 064419 |
| 49619 | 128693587 | 128695912 | 064419 |
| 49620 | 128740379 | 128741455 | |
| 49621 | 128742428 | 128743338 | |
| 49622 | 128784314 | 128785168 | 158457 |
| 49623 | 128828128 | 128829614 | 128602 |
| 49624 | 128864081 | 128866181 | 158467 |
| 49625 | 129027236 | 129027892 | 158467 |
| 49626 | 129141993 | 129142920 | 240204 |
| 49627 | 129170425 | 129172188 | |
| 49628 | 129209308 | 129210226 | |
| 49629 | 129248980 | 129252901 | 106459, 273329 |
| 49630 | 129419589 | 129420500 | |
| 49631 | 129464610 | 129466643 | |
| 49632 | 129469283 | 129472572 | 186591 |
| 49633 | 129491146 | 129494848 | 186591 |
| 49634 | 129516752 | 129518471 | 186591 |
| 49635 | 129526008 | 129526865 | 186591 |
| 49636 | 129546076 | 129547713 | 186591 |
| 49637 | 129588554 | 129593423 | 244036, 186591 |
| 49638 | 129598275 | 129599644 | 244036 |
| 49639 | 129648137 | 129651193 | 244036 |
| 49640 | 129690325 | 129691735 | 091732 |
| 49641 | 129703416 | 129704862 | |
| 49642 | 129705482 | 129706068 | |
| 49643 | 129709017 | 129711428 | 128607 |
| 49644 | 129844369 | 129845902 | 240571, 146842 |
| 49645 | 130080349 | 130081523 | 106477 |
| 49646 | 130125310 | 130127091 | 106484, 272701 |
| 49647 | 130352783 | 130354085 | 213265, 158623 |
| 49648 | 130568983 | 130570742 | 226380, 233559 |
| 49649 | 130570833 | 130573546 | 226380, 233559 |
| 49650 | 130574903 | 130576741 | 226380, 233559 |
| 49651 | 130577262 | 130580047 | 226380, 233559 |
| 49652 | 130583530 | 130591919 | 226380, 233559 |
| 49653 | 130593399 | 130604714 | 226380, 233559 |
| 49654 | 130606884 | 130611284 | 233559 |
| 49655 | 130614920 | 130615582 | 271204 |
| 49656 | 130623710 | 130629483 | 231721, 273319 |
| 49657 | 130632330 | 130633037 | 231721 |
| 49658 | 130679051 | 130679793 | 231721 |
| 49659 | 130680909 | 130682047 | 231721 |
| 49660 | 130696769 | 130697804 | 231721 |
| 49661 | 130741844 | 130742662 | 231721 |
| 49662 | 130746541 | 130746929 | 231721 |
| 49663 | 130772879 | 130774357 | 231721 |
| 49664 | 130790114 | 130796602 | 128585, 231721 |
| 49665 | 130848590 | 130849621 | 128585 |
| 49666 | 130872134 | 130873839 | 128585 |
| 49667 | 130903882 | 130904999 | 128585 |
| 49668 | 130910198 | 130911850 | 128585 |
| 49669 | 130922882 | 130923994 | 128585 |
| 49670 | 130933619 | 130934477 | 128585 |
| 49671 | 131011585 | 131014414 | 236753, 128585 |
| 49672 | 131085271 | 131086298 | 128585 |
| 49673 | 131103302 | 131103881 | 128585 |
| 49674 | 131198094 | 131198641 | 128567 |
| 49675 | 132351274 | 132351843 | 183470 |
| 49676 | 132765769 | 132767458 | 106554 |
| 49677 | 132937571 | 132938736 | 131558 |
| 49678 | 133455175 | 133456404 | 131558 |
| 49679 | 133859482 | 133859970 | 155530 |
| 49680 | 134000326 | 134002258 | 205060 |
| 49681 | 134038133 | 134038991 | |
| 49682 | 134040337 | 134041338 | |
| 49683 | 134046652 | 134053148 | |
| 49684 | 134053372 | 134054727 | |
| 49685 | 134106837 | 134107917 | 273297 |
| 49686 | 134117214 | 134118040 | |
| 49687 | 134247383 | 134248332 | 227471 |
| 49688 | 134277931 | 134279570 | |
| 49689 | 134327242 | 134328590 | |
| 49690 | 134330514 | 134332405 | 172331 |
| 49691 | 134387739 | 134389527 | |
| 49692 | 134528821 | 134530744 | 122786 |
| 49693 | 134570440 | 134572430 | 122786 |
| 49694 | 134575095 | 134579060 | 122786 |
| 49695 | 134583826 | 134588628 | 122786 |
| 49696 | 134594332 | 134596448 | 122786 |
| 49697 | 134625874 | 134627128 | 122786 |
| 49698 | 134670847 | 134672153 | 146856 |
| 49699 | 134676350 | 134677925 | 146856 |
| 49700 | 134831629 | 134834068 | 229974, 146859, 146856, |
| | | | 122783 |
| 49701 | 134844427 | 134845895 | 146859, 122783 |
| 49702 | 134853090 | 134857402 | 272941, 122783 |
| 49703 | 134895396 | 134897174 | 105875 |
| 49704 | 134902488 | 134903270 | |
| 49705 | 134938134 | 134939726 | 146857 |
| 49706 | 135126497 | 135126910 | 080802 |
| 49707 | 135193142 | 135195536 | 080802 |
| 49708 | 135242084 | 135243610 | 155561 |
| 49709 | 135346519 | 135348342 | 243317 |
| 49710 | 135458222 | 135459612 | |
| 49711 | 135661224 | 135662600 | 105887 |
| 49712 | 136928434 | 136929111 | 105894 |
| 49713 | 137524994 | 137525587 | 157680 |
| 49714 | 137633116 | 137634867 | 182158 |
| 49715 | 137684771 | 137687511 | 122787, 182158 |
| 49716 | 137714934 | 137716432 | 122787 |
| 49717 | 137734200 | 137735121 | 122787 |
| 49718 | 137749478 | 137750407 | 122787 |
| 49719 | 137751714 | 137753063 | 122787 |
| 49720 | 137758704 | 137759506 | 122787 |
| 49721 | 137770911 | 137772499 | 122787 |
| 49722 | 137773951 | 137775085 | 122787 |
| 49723 | 137780943 | 137781606 | 122787 |
| 49724 | 137782030 | 137782544 | 122787 |
| 49725 | 137843320 | 137844069 | |
| 49726 | 137919215 | 137920642 | |
| 49727 | 137922574 | 137923232 | |
| 49728 | 137927807 | 137928880 | |
| 49729 | 137929510 | 137930932 | |
| 49730 | 138000898 | 138001889 | |
| 49731 | 138073355 | 138074461 | |
| 49732 | 138140259 | 138140717 | |
| 49733 | 138144217 | 138146448 | 122779 |
| 49734 | 138162922 | 138163832 | 122779 |
| 49735 | 138197537 | 138198143 | 122779 |
| 49736 | 138247866 | 138251550 | 122779 |
| 49737 | 138412178 | 138412935 | 105929 |
| 49738 | 138437159 | 138437952 | 105929 |
| 49739 | 138575057 | 138576247 | 122778 |
| 49740 | 138645768 | 138646313 | 122778 |
| 49741 | 138665679 | 138666704 | 122778 |
| 49742 | 138760549 | 138761295 | 105939 |
| 49743 | 138792504 | 138795450 | 105939 |
| 49744 | 138802787 | 138803540 | |
| 49745 | 138818110 | 138818642 | 105948 |
| 49746 | 138915177 | 138918004 | 157741 |
| 49747 | 138926759 | 138929062 | 157741 |
| 49748 | 139024470 | 139027415 | 269955, 146963, 164898 |
| 49749 | 139043241 | 139046157 | 273391, 269955, 146963 |
| 49750 | 139156574 | 139157259 | 188883 |
| 49751 | 139325997 | 139326834 | 064393 |
| 49752 | 139333738 | 139334714 | 064393 |
| 49753 | 139343548 | 139344127 | 064393 |
| 49754 | 139344521 | 139345677 | 064393 |
| 49755 | 139356657 | 139361196 | 064393 |
| 49756 | 139364010 | 139368811 | 064393 |
| 49757 | 139375836 | 139376656 | 064393 |
| 49758 | 139423048 | 139424192 | 064393 |
| 49759 | 139424694 | 139427777 | 064393 |
| 49760 | 139429895 | 139439212 | 064393 |
| 49761 | 139439420 | 139442116 | 064393 |
| 49762 | 139443093 | 139445965 | 064393 |
| 49763 | 139452205 | 139454217 | 064393 |
| 49764 | 139457544 | 139458513 | 064393 |
| 49765 | 139471125 | 139472566 | 064393 |
| 49766 | 139472747 | 139479097 | 059377, 064393 |
| 49767 | 139575663 | 139576418 | 059377 |
| 49768 | 139614937 | 139617766 | 059377 |
| 49769 | 139748693 | 139750514 | 059378 |
| 49770 | 139759028 | 139763718 | 059378 |
| 49771 | 139873946 | 139874461 | 006459 |
| 49772 | 139874889 | 139878014 | 260231, 006459 |
| 49773 | 140097498 | 140098643 | 157800 |
| 49774 | 140103492 | 140104188 | 146955, 157800 |
| 49775 | 140177918 | 140179635 | 133606 |
| 49776 | 140266939 | 140267835 | 146966 |
| 49777 | 140327657 | 140328520 | 146966 |
| 49778 | 140372390 | 140373647 | 133597, 146966 |
| 49779 | 140396029 | 140398084 | 240889, 133597, 090266 |
| 49780 | 140612916 | 140613361 | 157764 |
| 49781 | 140622877 | 140625454 | 157764 |
| 49782 | 140675579 | 140676335 | |
| 49783 | 140713976 | 140715198 | 090263 |
| 49784 | 140741772 | 140742935 | |
| 49785 | 140744647 | 140746119 | |
| 49786 | 141062304 | 141062870 | 261115 |
| 49787 | 141069015 | 141069728 | 261115 |
| 49788 | 141162669 | 141163316 | 261115 |
| 49789 | 141250585 | 141251868 | 006530, 261570 |
| 49790 | 141285150 | 141286807 | 006530 |
| 49791 | 141362099 | 141364147 | 270157, 257093 |
| 49792 | 141385323 | 141386068 | 257093 |
| 49793 | 141395380 | 141396396 | 257093 |
| 49794 | 141401155 | 141402575 | 257093 |
| 49795 | 141437027 | 141439184 | 106028, 228775 |
| 49796 | 141675487 | 141676581 | 257335 |
| 49797 | 141729836 | 141730938 | 257335 |
| 49798 | 141787490 | 141788451 | 257335 |
| 49799 | 142190915 | 142191482 | 211739 |
| 49800 | 142551779 | 142552903 | 106123 |
| 49801 | 142606758 | 142607764 | 127412 |
| 49802 | 142936870 | 142938220 | |
| 49803 | 142945671 | 142946748 | 197448 |
| 49804 | 142955799 | 142956595 | 231840, 197448 |
| 49805 | 142956675 | 142957399 | 231840, 197448 |
| 49806 | 142960149 | 142961676 | 231840, 197448 |
| 49807 | 142981546 | 142983031 | 231840, 178826 |
| 49808 | 142984850 | 142986437 | 106144, 178826 |
| 49809 | 143015561 | 143016346 | 188037 |
| 49810 | 143076525 | 143080214 | 232533, 159840 |
| 49811 | 143088363 | 143089230 | 146904 |
| 49812 | 143090228 | 143091199 | 146904 |
| 49813 | 143092218 | 143094589 | 146904 |
| 49814 | 143131649 | 143132238 | 229153 |
| 49815 | 143133161 | 143135176 | 234066, 229153 |
| 49816 | 143184462 | 143185260 | 229153 |
| 49817 | 143188974 | 143189534 | 229153 |
| 49818 | 143209772 | 143210411 | 229153 |
| 49819 | 143578962 | 143580435 | 198420 |
| 49820 | 143596721 | 143599736 | 198420 |
| 49821 | 143603949 | 143604387 | |
| 49822 | 143675875 | 143678888 | 270729, 273377, 243583 |
| 49823 | 144531816 | 144533796 | 196511 |
| 49824 | 147850510 | 147851214 | 174469 |
| 49825 | 148024296 | 148025086 | 174469 |
| 49826 | 148036273 | 148036991 | 174469 |
| 49827 | 148077555 | 148079556 | 174469 |
| 49828 | 148125601 | 148126054 | |
| 49829 | 148139989 | 148140680 | |
| 49830 | 148144296 | 148145629 | |
| 49831 | 148177868 | 148178900 | |
| 49832 | 148224606 | 148225376 | |
| 49833 | 148253694 | 148254717 | |
| 49834 | 148263000 | 148264592 | |
| 49835 | 148312088 | 148313040 | 170279 |
| 49836 | 148317210 | 148319244 | |
| 49837 | 148319695 | 148321488 | |
| 49838 | 148394843 | 148398180 | 055130, 273314 |
| 49839 | 148580118 | 148582745 | 106462 |
| 49840 | 148637084 | 148639626 | 252310 |
| 49841 | 148723664 | 148726288 | 155660 |
| 49842 | 148762035 | 148763389 | |
| 49843 | 148787327 | 148789049 | 197362 |
| 49844 | 148822325 | 148824737 | 240877, 197024, 204947 |
| 49845 | 148843321 | 148846074 | 197024 |
| 49846 | 148857421 | 148859026 | 197024 |
| 49847 | 148892157 | 148894231 | 170265 |
| 49848 | 148936174 | 148937816 | 170260 |
| 49849 | 148958278 | 148960230 | 204946 |
| 49850 | 149119015 | 149121034 | 261842 |
| 49851 | 149156197 | 149158731 | 196453 |
| 49852 | 149193660 | 149195426 | 181220 |
| 49853 | 149320777 | 149322680 | 133624 |
| 49854 | 149411481 | 149412689 | 133619 |
| 49855 | 149416994 | 149417980 | 133619 |
| 49856 | 149468454 | 149469414 | 181444 |
| 49857 | 149535109 | 149536278 | 106479 |
| 49858 | 149569450 | 149571439 | 171130, 204934 |
| 49859 | 149582095 | 149583373 | |
| 49860 | 150013211 | 150013761 | 106526 |
| 49861 | 150017439 | 150018554 | 106526 |
| 49862 | 150018619 | 150022771 | 127399, 106526 |
| 49863 | 150036960 | 150039286 | 261305, 106538 |
| 49864 | 150052260 | 150053159 | |
| 49865 | 150064442 | 150068135 | 214022, 196456, 240449 |
| 49866 | 150074783 | 150077256 | 239377, 196456 |
| 49867 | 150085271 | 150086819 | 196456 |
| 49868 | 150101293 | 150103073 | 196456 |
| 49869 | 150103622 | 150104815 | 196456 |
| 49870 | 150382277 | 150383531 | 106560 |
| 49871 | 150424235 | 150425174 | 196329 |
| 49872 | 150435750 | 150436561 | 196329 |
| 49873 | 150474969 | 150476461 | 241134 |
| 49874 | 150476793 | 150478801 | 241134 |
| 49875 | 150480528 | 150483535 | 241134 |
| 49876 | 150484573 | 150486531 | 241134 |
| 49877 | 150495642 | 150498979 | 002933, 106565 |
| 49878 | 150522967 | 150523886 | 002726 |
| 49879 | 150725052 | 150726511 | 243433, 197150 |
| 49880 | 150755041 | 150757925 | 164885, 164889 |
| 49881 | 150759325 | 150760811 | 164889 |
| 49882 | 150777023 | 150781136 | 164897, 164896 |
| 49883 | 150782668 | 150787072 | 133612 |
| 49884 | 150923384 | 150924973 | 033050 |
| 49885 | 150929061 | 150931204 | 033100 |
| 49886 | 150945166 | 150946586 | 243018, 082014 |
| 49887 | 151038655 | 151039835 | 013374 |
| 49888 | 151042836 | 151043863 | 013374 |
| 49889 | 151046331 | 151047742 | 013374 |
| 49890 | 151101117 | 151101875 | 187260 |
| 49891 | 151112745 | 151113296 | |
| 49892 | 151145196 | 151146203 | |
| 49893 | 151156325 | 151158085 | |
| 49894 | 151188770 | 151189359 | 106615 |
| 49895 | 151191772 | 151192794 | 106615 |
| 49896 | 151201026 | 151201779 | 106615 |
| 49897 | 151214723 | 151218480 | 106615 |
| 49898 | 151249422 | 151250015 | |
| 49899 | 151296857 | 151297454 | 106617 |
| 49900 | 151328026 | 151330455 | 106617 |
| 49901 | 151337123 | 151338498 | 106617 |
| 49902 | 151344726 | 151345684 | 106617 |
| 49903 | 151347274 | 151349582 | 106617 |
| 49904 | 151383940 | 151385992 | 106617 |
| 49905 | 151391502 | 151394351 | 106617 |
| 49906 | 151402709 | 151403432 | 106617 |
| 49907 | 151407403 | 151410011 | 106617 |
| 49908 | 151414324 | 151415318 | 106617 |
| 49909 | 151433391 | 151434048 | 106617 |
| 49910 | 151439579 | 151447152 | 106617 |
| 49911 | 151451042 | 151454069 | 106617 |
| 49912 | 151486451 | 151487156 | 106617 |
| 49913 | 151489669 | 151500288 | 106617 |
| 49914 | 151502665 | 151504779 | 242048, 106617 |
| 49915 | 151505023 | 151506295 | 242048, 106617 |
| 49916 | 151515199 | 151517550 | 106617 |
| 49917 | 151517727 | 151521720 | 106617 |
| 49918 | 151540417 | 151543337 | 106617 |
| 49919 | 151547950 | 151549383 | 106617 |
| 49920 | 151551446 | 151551939 | 106617 |
| 49921 | 151564682 | 151568965 | 106617 |
| 49922 | 151571910 | 151575749 | 239911, 106617 |
| 49923 | 151594124 | 151595078 | |
| 49924 | 151614302 | 151616304 | |
| 49925 | 151620083 | 151624103 | |
| 49926 | 151722021 | 151723767 | 178234 |
| 49927 | 151751893 | 151752407 | 178234 |
| 49928 | 152038200 | 152038967 | 055609 |
| 49929 | 152133268 | 152133665 | |
| 49930 | 152133758 | 152134195 | 241735 |
| 49931 | 152160041 | 152162317 | 261455 |
| 49932 | 152219642 | 152220130 | |
| 49933 | 152373173 | 152373916 | 196584 |
| 49934 | 152456317 | 152459389 | 133627 |
| 49935 | 152596392 | 152597490 | |
| 49936 | 154636548 | 154637232 | 236408, 130226 |
| 49937 | 154705182 | 154705911 | |
| 49938 | 154719202 | 154721203 | 214106 |
| 49939 | 154751482 | 154752488 | 157212 |
| 49940 | 154793534 | 154796656 | 273344, 157212 |
| 49941 | 154861607 | 154862107 | 157219, 220575 |
| 49942 | 155011569 | 155012107 | |
| 49943 | 155013566 | 155014125 | |
| 49944 | 155025228 | 155028700 | |
| 49945 | 155043138 | 155046621 | |
| 49946 | 155048665 | 155050371 | |
| 49947 | 155061298 | 155062468 | |
| 49948 | 155067604 | 155069711 | |
| 49949 | 155082035 | 155083992 | |
| 49950 | 155087395 | 155091681 | 273117, 186480 |
| 49951 | 155094667 | 155095386 | 186480 |
| 49952 | 155098368 | 155099807 | 186480 |
| 49953 | 155136990 | 155138268 | |
| 49954 | 155288716 | 155289309 | 146910 |
| 49955 | 155339435 | 155340741 | |
| 49956 | 155387538 | 155388261 | |
| 49957 | 155389750 | 155391054 | |
| 49958 | 155410897 | 155413394 | 216895 |
| 49959 | 155436085 | 155438295 | 184863, 216895 |
| 49960 | 155444297 | 155447173 | 184863 |
| 49961 | 155474487 | 155475881 | 184863 |
| 49962 | 155508035 | 155508653 | 184863 |
| 49963 | 155599745 | 155603194 | 164690 |
| 49964 | 155604284 | 155604643 | 164690 |
| 49965 | 155651319 | 155652250 | |
| 49966 | 155653045 | 155655254 | |
| 49967 | 155709409 | 155712962 | |
| 49968 | 155742860 | 155746002 | |
| 49969 | 155902644 | 155903067 | |
| 49970 | 155939588 | 155940496 | |
| 49971 | 156160568 | 156161513 | |
| 49972 | 156172187 | 156173801 | |
| 49973 | 156249263 | 156249851 | |
| 49974 | 156265893 | 156267282 | 182648 |
| 49975 | 156268392 | 156268948 | 182648 |
| 49976 | 156307753 | 156310332 | 182648 |
| 49977 | 156319484 | 156321025 | 182648 |
| 49978 | 156337561 | 156338201 | 182648 |
| 49979 | 156401488 | 156402209 | 182648 |
| 49980 | 156407172 | 156409928 | 182648 |
| 49981 | 156432425 | 156434452 | 105982, 182648, 244291 |
| 49982 | 156517672 | 156518279 | 105983 |
| 49983 | 156582693 | 156583425 | 105983 |
| 49984 | 156684635 | 156686984 | 105983 |
| 49985 | 156741957 | 156743621 | 146909 |
| 49986 | 156930940 | 156933075 | 009335 |
| 49987 | 157046533 | 157046945 | 009335 |
| 49988 | 157063863 | 157066657 | |
| 49989 | 157128629 | 157131037 | 105993 |
| 49990 | 157646923 | 157647403 | 233038, 155093 |
| 49991 | 158056129 | 158057348 | 155093 |
| 49992 | 158441497 | 158442523 | 146918 |
| 49993 | 158497078 | 158498460 | 146918 |
| 49994 | 158594647 | 158596142 | 117868 |
| 49995 | 158609900 | 158612651 | 117868 |
| 49996 | 158620713 | 158623525 | 117868 |
| 49997 | 158648895 | 158649774 | 126870 |
| 49998 | 158656003 | 158657401 | 126870 |

The locations of ODSCs on Chromosome 8 are shown in Table 50.

TABLE 50
ODSC Locations on Chromosome 8

| SEQ ID NO. | Start | End | Overlapping Gene(s) |
|---|---|---|---|
| 49999 | 438143 | 439346 | |
| 50000 | 440302 | 440785 | 180190 |
| 50001 | 493920 | 496229 | 180190 |
| 50002 | 680314 | 682098 | 104714 |
| 50003 | 1649097 | 1649926 | 198010 |
| 50004 | 1710756 | 1711662 | 253982, 182372 |
| 50005 | 1711748 | 1713471 | 253982, 182372 |
| 50006 | 1842978 | 1843536 | 104728 |
| 50007 | 1894890 | 1895931 | 104728 |
| 50008 | 1907611 | 1909034 | |
| 50009 | 1921211 | 1922997 | 176595, 270988, 253764, |
| | | | 253696 |
| 50010 | 1948980 | 1950324 | 176595 |
| 50011 | 3844944 | 3845731 | 183117 |
| 50012 | 3958218 | 3959577 | 183117 |
| 50013 | 3961140 | 3961933 | 183117 |
| 50014 | 4188716 | 4189746 | 183117 |
| 50015 | 4195700 | 4196519 | 183117 |
| 50016 | 4680206 | 4680774 | 183117 |
| 50017 | 4783679 | 4784667 | 183117 |
| 50018 | 6263133 | 6265190 | 147316, 246089 |
| 50019 | 6373722 | 6374441 | 147316, 091879 |
| 50020 | 6565450 | 6567203 | 155189, 249898 |
| 50021 | 6698084 | 6699240 | 245857 |
| 50022 | 6734556 | 6736547 | 164825, 245857 |
| 50023 | 6757809 | 6759232 | |
| 50024 | 6766578 | 6768179 | |
| 50025 | 8100670 | 8101213 | 253981, 173295 |
| 50026 | 8126010 | 8128427 | |
| 50027 | 8145784 | 8147286 | |
| 50028 | 8153057 | 8154162 | |
| 50029 | 8168099 | 8168710 | |
| 50030 | 8242680 | 8244972 | 182319 |
| 50031 | 8284813 | 8287277 | |
| 50032 | 8313195 | 8314189 | |
| 50033 | 8324239 | 8325581 | |
| 50034 | 8388078 | 8388859 | |
| 50035 | 8456880 | 8459318 | |
| 50036 | 8537633 | 8538288 | |
| 50037 | 8558611 | 8560312 | 176305, 253958 |
| 50038 | 8608060 | 8609041 | 254367 |
| 50039 | 8621186 | 8622052 | 254367 |
| 50040 | 8629674 | 8631070 | 254367 |
| 50041 | 8632633 | 8633168 | 254367 |
| 50042 | 8662948 | 8664713 | 147324 |
| 50043 | 8700856 | 8702737 | 147324 |
| 50044 | 8729491 | 8730144 | 147324 |
| 50045 | 8745491 | 8747033 | 147324 |
| 50046 | 8749409 | 8752101 | 147324 |
| 50047 | 8774864 | 8775727 | |
| 50048 | 8852120 | 8852973 | |
| 50049 | 8855990 | 8856459 | |
| 50050 | 8859495 | 8861275 | 104626 |
| 50051 | 8864041 | 8864622 | 104626 |
| 50052 | 8869256 | 8870696 | 104626 |
| 50053 | 8880622 | 8881309 | 104626 |
| 50054 | 8931780 | 8933530 | 104626 |
| 50055 | 8953152 | 8954227 | 104626 |
| 50056 | 8991473 | 8994493 | 173281 |
| 50057 | 9004959 | 9006782 | 173281 |
| 50058 | 9006819 | 9010941 | 253426, 173281 |
| 50059 | 9045364 | 9045985 | |
| 50060 | 9104886 | 9106855 | |
| 50061 | 9169508 | 9170295 | 254235 |
| 50062 | 9174382 | 9178903 | 254235 |
| 50063 | 9181040 | 9185480 | 254235 |
| 50064 | 9195211 | 9196281 | 254235 |
| 50065 | 9204048 | 9205641 | 254235 |
| 50066 | 9211456 | 9212647 | 254235, 253735 |
| 50067 | 9304691 | 9309367 | |
| 50068 | 9369613 | 9371001 | |
| 50069 | 9413132 | 9414400 | 173273, 272267 |
| 50070 | 9638745 | 9640538 | 173273 |
| 50071 | 9903545 | 9904622 | |
| 50072 | 9911346 | 9913370 | 175806, 260093 |
| 50073 | 9944555 | 9945586 | 221320, 175806 |
| 50074 | 9995898 | 9997088 | 175806 |
| 50075 | 10008793 | 10011336 | 175806 |
| 50076 | 10122545 | 10123395 | 175806 |
| 50077 | 10167934 | 10168771 | 175806 |
| 50078 | 10191938 | 10192822 | 175806 |
| 50079 | 10253022 | 10253405 | 175806 |
| 50080 | 10275235 | 10275715 | 175806 |
| 50081 | 10427434 | 10428283 | |
| 50082 | 10437066 | 10437951 | |
| 50083 | 10487848 | 10488584 | 183638 |
| 50084 | 10510454 | 10510914 | 183638 |
| 50085 | 10538243 | 10538945 | 183638, 171060 |
| 50086 | 10548329 | 10549359 | 183638, 171060 |
| 50087 | 10617263 | 10618893 | 258724, 248896, 171056 |
| 50088 | 10656086 | 10659393 | 258724, 254093, 171056 |
| 50089 | 10673311 | 10674551 | 258724, 254093, 171056 |
| 50090 | 10696195 | 10697759 | 258724, 254093, 171056 |
| 50091 | 11141168 | 11143108 | 104643 |
| 50092 | 11314391 | 11316393 | 154319 |
| 50093 | 11323617 | 11324676 | 154319 |
| 50094 | 11331480 | 11332965 | 154319 |
| 50095 | 11471858 | 11472805 | |
| 50096 | 11485901 | 11487121 | |
| 50097 | 11488941 | 11489478 | |
| 50098 | 11499397 | 11500077 | |
| 50099 | 11520116 | 11520573 | |
| 50100 | 11528673 | 11529261 | |
| 50101 | 11560081 | 11563241 | 136574 |
| 50102 | 11568474 | 11568964 | 136574 |
| 50103 | 11574646 | 11577198 | 136574 |
| 50104 | 11587709 | 11588692 | 136574 |
| 50105 | 11599722 | 11600578 | 136574 |
| 50106 | 11626903 | 11628013 | 154328 |
| 50107 | 11657448 | 11662117 | 255046, 079459 |
| 50108 | 11664842 | 11666851 | 079459 |
| 50109 | 11670884 | 11672099 | 079459 |
| 50110 | 11709991 | 11711915 | 164733 |
| 50111 | 11719901 | 11726520 | 164733 |
| 50112 | 11753956 | 11756439 | 255144 |
| 50113 | 12583079 | 12584941 | 266206, 154359 |
| 50114 | 12610863 | 12614092 | 154359 |
| 50115 | 12808501 | 12809651 | 250305 |
| 50116 | 12850887 | 12851290 | 250305 |
| 50117 | 12885477 | 12886178 | 250305 |
| 50118 | 12988758 | 12989731 | 164741 |
| 50119 | 12989807 | 12991831 | 164741 |
| 50120 | 13015356 | 13017652 | 164741 |
| 50121 | 13050229 | 13052702 | 164741 |
| 50122 | 13067535 | 13068242 | 164741 |
| 50123 | 13078087 | 13079057 | 164741 |
| 50124 | 13088623 | 13089005 | 164741 |
| 50125 | 13097042 | 13097556 | 164741 |
| 50126 | 13103788 | 13106128 | 164741 |
| 50127 | 13123155 | 13123804 | 164741 |
| 50128 | 13127818 | 13129743 | 164741 |
| 50129 | 13132177 | 13134989 | 164741 |
| 50130 | 13187208 | 13188493 | 164741 |
| 50131 | 13210203 | 13212247 | 253257, 164741 |
| 50132 | 13214728 | 13215422 | 164741 |
| 50133 | 13223697 | 13225223 | 164741 |
| 50134 | 13229691 | 13231158 | 164741 |
| 50135 | 13315626 | 13316602 | 164741 |
| 50136 | 15513242 | 15514505 | 104723 |
| 50137 | 16683446 | 16684009 | 253496 |
| 50138 | 16688951 | 16690151 | 253496 |
| 50139 | 16883634 | 16885988 | 155970 |
| 50140 | 16889427 | 16890844 | 155970 |
| 50141 | 16906694 | 16907417 | 155970 |
| 50142 | 16908706 | 16909580 | 155970 |
| 50143 | 17012901 | 17014996 | 104219 |
| 50144 | 17058114 | 17058703 | 104219 |
| 50145 | 17082393 | 17085455 | 271948 |
| 50146 | 17103737 | 17105511 | 155975, 198791 |
| 50147 | 17178618 | 17179652 | 003987 |
| 50148 | 17185494 | 17187326 | 003987 |
| 50149 | 17191603 | 17196284 | 003987 |
| 50150 | 17198432 | 17199148 | 003987 |
| 50151 | 17204690 | 17209709 | 003987 |
| 50152 | 17323038 | 17325616 | |
| 50153 | 17347132 | 17347777 | |
| 50154 | 17350996 | 17357361 | 003989 |
| 50155 | 17364038 | 17364487 | 003989 |
| 50156 | 17382261 | 17384368 | 003989 |
| 50157 | 17433958 | 17435379 | 104213 |
| 50158 | 17465631 | 17466163 | 104213 |
| 50159 | 17471714 | 17472841 | 104213 |
| 50160 | 17517987 | 17520105 | 129422 |
| 50161 | 17520190 | 17520949 | 129422 |
| 50162 | 17523173 | 17523767 | 129422 |
| 50163 | 17532237 | 17532837 | 129422 |
| 50164 | 17533314 | 17534439 | 129422 |
| 50165 | 17623417 | 17625919 | 129422 |
| 50166 | 17644106 | 17645172 | 129422 |
| 50167 | 17645801 | 17647381 | 129422 |
| 50168 | 17652072 | 17653015 | 129422 |
| 50169 | 17657476 | 17663904 | 253944, 129422 |
| 50170 | 17676577 | 17679263 | 253671, 253944 |
| 50171 | 17706843 | 17708938 | 253944 |
| 50172 | 17752564 | 17754680 | 104760 |
| 50173 | 17779247 | 17782077 | 078674 |
| 50174 | 17833903 | 17834373 | 078674 |
| 50175 | 17940553 | 17943181 | 245281, 104763 |
| 50176 | 18027415 | 18029871 | 171428 |
| 50177 | 18041879 | 18043392 | 171428 |
| 50178 | 18237978 | 18238803 | |
| 50179 | 18244318 | 18245555 | 271654 |
| 50180 | 18248449 | 18249383 | 156006 |
| 50181 | 18500390 | 18501354 | 156011 |
| 50182 | 18518040 | 18519074 | 156011 |
| 50183 | 18523189 | 18524581 | 156011 |
| 50184 | 18574824 | 18579146 | 187229, 156011 |
| 50185 | 18611102 | 18611598 | 156011 |
| 50186 | 18662065 | 18662502 | 156011 |
| 50187 | 18703864 | 18704990 | 156011 |
| 50188 | 18745500 | 18746439 | 156011 |
| 50189 | 18811662 | 18813239 | 156011 |
| 50190 | 18813323 | 18816261 | 156011 |
| 50191 | 18824941 | 18825657 | 156011 |
| 50192 | 18828539 | 18829023 | 156011 |
| 50193 | 18834787 | 18837355 | 201157, 156011 |
| 50194 | 18865460 | 18866175 | 156011 |
| 50195 | 18867249 | 18869101 | 156011 |
| 50196 | 18869281 | 18872547 | 156011 |
| 50197 | 18924783 | 18925677 | 156011 |
| 50198 | 18950683 | 18952164 | 253557, 254242 |
| 50199 | 18984128 | 18986521 | 253557 |
| 50200 | 19041461 | 19042107 | 253557 |
| 50201 | 19124752 | 19125791 | |
| 50202 | 19128617 | 19129722 | |
| 50203 | 19131092 | 19131806 | |
| 50204 | 19135591 | 19136931 | |
| 50205 | 19148802 | 19149693 | |
| 50206 | 19169902 | 19172285 | 104611 |
| 50207 | 19175798 | 19177159 | 104611 |
| 50208 | 19177879 | 19178706 | 104611 |
| 50209 | 19226193 | 19227290 | 104611 |
| 50210 | 19246468 | 19247434 | 104611 |
| 50211 | 19247745 | 19248646 | 104611 |
| 50212 | 19518393 | 19519242 | 147408 |
| 50213 | 19520917 | 19521695 | 147408 |
| 50214 | 19522299 | 19523493 | 147408 |
| 50215 | 19535236 | 19536751 | 253270, 147408 |
| 50216 | 19674185 | 19676021 | 104613 |
| 50217 | 20054472 | 20056296 | 147416 |
| 50218 | 20058267 | 20058687 | 147416 |
| 50219 | 20079767 | 20080414 | 147416 |
| 50220 | 20125599 | 20126864 | 061337 |
| 50221 | 21349384 | 21350454 | |
| 50222 | 21423880 | 21424430 | |
| 50223 | 21541427 | 21543059 | |
| 50224 | 21776105 | 21778243 | 130227 |
| 50225 | 21867071 | 21868682 | |
| 50226 | 21905698 | 21907463 | 158856, 158815 |
| 50227 | 21910965 | 21912042 | 158856 |
| 50228 | 21916334 | 21917089 | 158856 |
| 50229 | 21917898 | 21918406 | 158856 |
| 50230 | 21923620 | 21924867 | 158856 |
| 50231 | 21946104 | 21948224 | 158863 |
| 50232 | 21965898 | 21968229 | 173566 |
| 50233 | 21998256 | 21999694 | 168476 |
| 50234 | 22000905 | 22001651 | |
| 50235 | 22021851 | 22023708 | 168487, 168484 |
| 50236 | 22027193 | 22028222 | 177725, 168487 |
| 50237 | 22036030 | 22037039 | 168487 |
| 50238 | 22042101 | 22042676 | 168487 |
| 50239 | 22051584 | 22052357 | 168487 |
| 50240 | 22062993 | 22063956 | 168487 |
| 50241 | 22077495 | 22078219 | 168490 |
| 50242 | 22085118 | 22085722 | 168490 |
| 50243 | 22100930 | 22103675 | 208037, 168495 |
| 50244 | 22131558 | 22132092 | 254064 |
| 50245 | 22144622 | 22146160 | 197181 |
| 50246 | 22213555 | 22217607 | 197181 |
| 50247 | 22221534 | 22226198 | 104635 |
| 50248 | 22227001 | 22228957 | 104635 |
| 50249 | 22240306 | 22242393 | 104635 |
| 50250 | 22242415 | 22243370 | 104635 |
| 50251 | 22244470 | 22247811 | 241026, 104635 |
| 50252 | 22248313 | 22251362 | 104635 |
| 50253 | 22268170 | 22270262 | 104635 |
| 50254 | 22297448 | 22300807 | 120910 |
| 50255 | 22373781 | 22374432 | 120910 |
| 50256 | 22413798 | 22414646 | 120896 |
| 50257 | 22417222 | 22418784 | 120896 |
| 50258 | 22421982 | 22424126 | 254230, 120896 |
| 50259 | 22436347 | 22441056 | 120913 |
| 50260 | 22443862 | 22444894 | 120913 |
| 50261 | 22445876 | 22447538 | 248235, 120913 |
| 50262 | 22452444 | 22453223 | 248235, 120913 |
| 50263 | 22453792 | 22456784 | 120913, 248235 |
| 50264 | 22461442 | 22463554 | 158941, 241852 |
| 50265 | 22493965 | 22494807 | 147439 |
| 50266 | 22525682 | 22527402 | 147439 |
| 50267 | 22542985 | 22543814 | |
| 50268 | 22772263 | 22774452 | 134020 |
| 50269 | 22851210 | 22853470 | 008853, 134020, 245025 |
| 50270 | 22856611 | 22857917 | 134020, 008853, 245025 |
| 50271 | 22871345 | 22872627 | 008853, 245025 |
| 50272 | 22921207 | 22923541 | 120889 |
| 50273 | 22924622 | 22927509 | 246130, 120889 |
| 50274 | 22931190 | 22933002 | 253616, 246130 |
| 50275 | 23019043 | 23019732 | 173530 |
| 50276 | 23019910 | 23021769 | 173530 |
| 50277 | 23038761 | 23040636 | |
| 50278 | 23075186 | 23076116 | 104689 |
| 50279 | 23081657 | 23083238 | 250714, 246582, 104689 |
| 50280 | 23103384 | 23104705 | 147457 |
| 50281 | 23145198 | 23146494 | 104679 |
| 50282 | 23211403 | 23211967 | 134013, 253837 |
| 50283 | 23314578 | 23315543 | 197217 |
| 50284 | 23364084 | 23364822 | |
| 50285 | 23385945 | 23387848 | 147454 |
| 50286 | 23395823 | 23397035 | 147454 |
| 50287 | 23405118 | 23405714 | 147454 |
| 50288 | 23407861 | 23409355 | 147454 |
| 50289 | 23410959 | 23412560 | 268085, 147454 |
| 50290 | 23417791 | 23419970 | 268085, 147454 |
| 50291 | 23450861 | 23451807 | |
| 50292 | 23539134 | 23540789 | 167034 |
| 50293 | 23618322 | 23620623 | 253471 |
| 50294 | 23896577 | 23897251 | |
| 50295 | 24206389 | 24207234 | 253535, 042980 |
| 50296 | 24228650 | 24229211 | 253535 |
| 50297 | 24244990 | 24245760 | 253535, 134028 |
| 50298 | 24387010 | 24388601 | 253535 |
| 50299 | 24424969 | 24426105 | 253535 |
| 50300 | 24430381 | 24431270 | 253535 |
| 50301 | 24432312 | 24435005 | 253535 |
| 50302 | 24579351 | 24580783 | 253535 |
| 50303 | 24856473 | 24858571 | 253832 |
| 50304 | 25041062 | 25046664 | 147459 |
| 50305 | 25052349 | 25053699 | 147459 |
| 50306 | 25055895 | 25057573 | 147459 |
| 50307 | 25071817 | 25073412 | 147459 |
| 50308 | 25087519 | 25088082 | 147459 |
| 50309 | 25165473 | 25166935 | 147459 |
| 50310 | 25175894 | 25178622 | 147459 |
| 50311 | 25186114 | 25186847 | 147459 |
| 50312 | 25275242 | 25276274 | 147459 |
| 50313 | 25314491 | 25317015 | 184661, 104756 |
| 50314 | 25991048 | 25992219 | |
| 50315 | 26007396 | 26010095 | |
| 50316 | 26013780 | 26015574 | |
| 50317 | 26148466 | 26151070 | 221914 |
| 50318 | 26152358 | 26153172 | 221914 |
| 50319 | 26153486 | 26154066 | 221914 |
| 50320 | 26186893 | 26187722 | 221914 |
| 50321 | 26239666 | 26242067 | 104765, 228451 |
| 50322 | 26305828 | 26308220 | 104765 |
| 50323 | 26308622 | 26309151 | 104765 |
| 50324 | 26315271 | 26315826 | 104765 |
| 50325 | 26339170 | 26340074 | 104765 |
| 50326 | 26428506 | 26430983 | 092964 |
| 50327 | 26433741 | 26436414 | 092964 |
| 50328 | 26438166 | 26438779 | 092964 |
| 50329 | 26438947 | 26439694 | 092964 |
| 50330 | 26472151 | 26472807 | 092964 |
| 50331 | 26473067 | 26474241 | 092964 |
| 50332 | 26493306 | 26493744 | 092964 |
| 50333 | 26505703 | 26506846 | 092964 |
| 50334 | 26557049 | 26559746 | |
| 50335 | 26563551 | 26564230 | |
| 50336 | 26611777 | 26612606 | 120907 |
| 50337 | 26621547 | 26622222 | 120907 |
| 50338 | 26696767 | 26699422 | 120907 |
| 50339 | 26699609 | 26701476 | 120907 |
| 50340 | 26718368 | 26718957 | 120907 |
| 50341 | 26721364 | 26725066 | 120907 |
| 50342 | 26935762 | 26937321 | |
| 50343 | 26941962 | 26943320 | |
| 50344 | 26946096 | 26948333 | |
| 50345 | 26948360 | 26949226 | |
| 50346 | 27029574 | 27030205 | |
| 50347 | 27107048 | 27108416 | 015592 |
| 50348 | 27131370 | 27132961 | |
| 50349 | 27137427 | 27140958 | |
| 50350 | 27155928 | 27158408 | 104228 |
| 50351 | 27165501 | 27170283 | 120899, 104228 |
| 50352 | 27180038 | 27182297 | 120899 |
| 50353 | 27190269 | 27193788 | 120899 |
| 50354 | 27197907 | 27198522 | 120899 |
| 50355 | 27218633 | 27221635 | 120899 |
| 50356 | 27297834 | 27298512 | 120899 |
| 50357 | 27316564 | 27317297 | 120903, 120899 |
| 50358 | 27345527 | 27346881 | |
| 50359 | 27347145 | 27349793 | 120915 |
| 50360 | 27440599 | 27443193 | 234770 |
| 50361 | 27452006 | 27454101 | |
| 50362 | 27454541 | 27455128 | 120885 |
| 50363 | 27460372 | 27461389 | 120885 |
| 50364 | 27467701 | 27475343 | 120885 |
| 50365 | 27477789 | 27478477 | |
| 50366 | 27490946 | 27491758 | 168077 |
| 50367 | 27559230 | 27561364 | 265075 |
| 50368 | 27607634 | 27608551 | 147419, 253875 |
| 50369 | 27629052 | 27631009 | 171320, 147419 |
| 50370 | 27631351 | 27632251 | 171320 |
| 50371 | 27695197 | 27695758 | 168078 |
| 50372 | 27729956 | 27730536 | 168079 |
| 50373 | 27859217 | 27859751 | |
| 50374 | 27927134 | 27927984 | 189233 |
| 50375 | 27929107 | 27930734 | 189233 |
| 50376 | 27934469 | 27936068 | 189233 |
| 50377 | 27949877 | 27951479 | 134014 |
| 50378 | 28112148 | 28113154 | 253690 |
| 50379 | 28198451 | 28199425 | 168081 |
| 50380 | 28222155 | 28225666 | 186918, 214050 |
| 50381 | 28226555 | 28227855 | 186918, 214050 |
| 50382 | 28230726 | 28231743 | 186918, 214050 |
| 50383 | 28235928 | 28237764 | 186918, 214050 |
| 50384 | 28238962 | 28240532 | 186918, 214050 |
| 50385 | 28241747 | 28244991 | 186918, 214050 |
| 50386 | 28259188 | 28260509 | 186918, 214050 |
| 50387 | 28268559 | 28270376 | 214050 |
| 50388 | 28347455 | 28348630 | 214050 |
| 50389 | 28351086 | 28352476 | 104290 |
| 50390 | 28479163 | 28480914 | 012232 |
| 50391 | 28482408 | 28482970 | 012232 |
| 50392 | 28514884 | 28516090 | 012232 |
| 50393 | 28526659 | 28528508 | 012232 |
| 50394 | 28558177 | 28561386 | 246339, 012232 |
| 50395 | 28584794 | 28585653 | 012232 |
| 50396 | 28657483 | 28658205 | 104299 |
| 50397 | 28746504 | 28749397 | 147421, 104299 |
| 50398 | 28843287 | 28843699 | 147421 |
| 50399 | 28845356 | 28846145 | 147421 |
| 50400 | 28860479 | 28860983 | 147421 |
| 50401 | 28874675 | 28875630 | 147421 |
| 50402 | 28974061 | 28974995 | 197892, 254129 |
| 50403 | 28982321 | 28984921 | 197892, 254129 |
| 50404 | 29027322 | 29028547 | 197892 |
| 50405 | 29082608 | 29083672 | 197892 |
| 50406 | 29087499 | 29088443 | 197892 |
| 50407 | 29119289 | 29121131 | 197892 |
| 50408 | 29140372 | 29141074 | |
| 50409 | 29208166 | 29209259 | 120875 |
| 50410 | 29316597 | 29317495 | |
| 50411 | 29355575 | 29356197 | |
| 50412 | 29594785 | 29596429 | 251191 |
| 50413 | 29614216 | 29614785 | 253490 |
| 50414 | 29624574 | 29625644 | 253490 |
| 50415 | 29626425 | 29629815 | 253490 |
| 50416 | 29630261 | 29632039 | 253490 |
| 50417 | 29832303 | 29833823 | |
| 50418 | 29876982 | 29878148 | |
| 50419 | 29909973 | 29910443 | |
| 50420 | 29939562 | 29941053 | 272256, 133872 |
| 50421 | 29946743 | 29947094 | |
| 50422 | 29952088 | 29954036 | 104660 |
| 50423 | 29957092 | 29957817 | 104660 |
| 50424 | 29970439 | 29971056 | 104660 |
| 50425 | 29994275 | 29995256 | 177669, 104660 |
| 50426 | 30012679 | 30015516 | 271869, 104671, 104660 |
| 50427 | 30085002 | 30085948 | |
| 50428 | 30086856 | 30087420 | |
| 50429 | 30109775 | 30111106 | |
| 50430 | 30111358 | 30113493 | |
| 50431 | 30113537 | 30115617 | |
| 50432 | 30140274 | 30140882 | |
| 50433 | 30169130 | 30169997 | |
| 50434 | 30171892 | 30172486 | |
| 50435 | 30172810 | 30173639 | |
| 50436 | 30239559 | 30244905 | 157110, 254109 |
| 50437 | 30248523 | 30249214 | 157110 |
| 50438 | 30271459 | 30272717 | 157110 |
| 50439 | 30314256 | 30315111 | 157110 |
| 50440 | 30357862 | 30362339 | 157110 |
| 50441 | 30366728 | 30367188 | 157110 |
| 50442 | 30385761 | 30386326 | 157110 |
| 50443 | 30388834 | 30390028 | 157110 |
| 50444 | 30404683 | 30405279 | 157110 |
| 50445 | 30407924 | 30409151 | 157110 |
| 50446 | 30428747 | 30429601 | 157110 |
| 50447 | 30440008 | 30440550 | 197265 |
| 50448 | 30458119 | 30458854 | 197265 |
| 50449 | 30473839 | 30474643 | 197265 |
| 50450 | 30514455 | 30516257 | 197265 |
| 50451 | 30568265 | 30568932 | 104687 |
| 50452 | 30583248 | 30586159 | 104687 |
| 50453 | 30600725 | 30602881 | 104691 |
| 50454 | 30659924 | 30663263 | 104695 |
| 50455 | 30664186 | 30665922 | 104695 |
| 50456 | 30668714 | 30670736 | 104695 |
| 50457 | 30747840 | 30748837 | 133863 |
| 50458 | 30890912 | 30892161 | 165392, 172733 |
| 50459 | 30932993 | 30933874 | 165392 |
| 50460 | 31976801 | 31977754 | 157168, 253974 |
| 50461 | 32176347 | 32176753 | 157168 |
| 50462 | 32191618 | 32192262 | 157168 |
| 50463 | 32284164 | 32284765 | 157168 |
| 50464 | 32290118 | 32290874 | 157168 |
| 50465 | 32403727 | 32407391 | 157168 |
| 50466 | 32409730 | 32410561 | 157168 |
| 50467 | 32410979 | 32412795 | 157168 |
| 50468 | 32572661 | 32573072 | 157168 |
| 50469 | 32582377 | 32583012 | 157168 |
| 50470 | 32610496 | 32611371 | 157168 |
| 50471 | 32732399 | 32733725 | |
| 50472 | 32741496 | 32744069 | |
| 50473 | 32830238 | 32830985 | |
| 50474 | 32863034 | 32864067 | 247134 |
| 50475 | 32891929 | 32893758 | 247134 |
| 50476 | 32947229 | 32950845 | |
| 50477 | 32955214 | 32957765 | |
| 50478 | 32961109 | 32964700 | |
| 50479 | 32970301 | 32971291 | |
| 50480 | 33099926 | 33102675 | |
| 50481 | 33171365 | 33173485 | |
| 50482 | 33193166 | 33194282 | |
| 50483 | 33209747 | 33212203 | |
| 50484 | 33265766 | 33266274 | 172728 |
| 50485 | 33328954 | 33331451 | 129696, 172728 |
| 50486 | 33341573 | 33343737 | 198042, 129696 |
| 50487 | 33369396 | 33372492 | 239039, 129696 |
| 50488 | 33423615 | 33424866 | 133874 |
| 50489 | 34783054 | 34783664 | |
| 50490 | 34870499 | 34872598 | |
| 50491 | 34879377 | 34881890 | |
| 50492 | 34899894 | 34901881 | |
| 50493 | 34963839 | 34964971 | |
| 50494 | 35344271 | 35345673 | 156687 |
| 50495 | 37002153 | 37002687 | |
| 50496 | 37311908 | 37312558 | 253161 |
| 50497 | 37373380 | 37374411 | 253414, 253161 |
| 50498 | 37451685 | 37452322 | |
| 50499 | 37548705 | 37549303 | |
| 50500 | 37552103 | 37554324 | 183779 |
| 50501 | 37593568 | 37595591 | 147475, 183154 |
| 50502 | 37619317 | 37621084 | 147471 |
| 50503 | 37638032 | 37640331 | |
| 50504 | 37706527 | 37707942 | 104221 |
| 50505 | 37756200 | 37757442 | 156675 |
| 50506 | 37760625 | 37762666 | |
| 50507 | 37773333 | 37774109 | |
| 50508 | 37853592 | 37854590 | |
| 50509 | 37887093 | 37890773 | 187840 |
| 50510 | 37894477 | 37894904 | 187840 |
| 50511 | 37901074 | 37901913 | 187840 |
| 50512 | 37914446 | 37916902 | 187840 |
| 50513 | 37924505 | 37925691 | |
| 50514 | 37961859 | 37963846 | 129691 |
| 50515 | 38033064 | 38035698 | 156735, 175324 |
| 50516 | 38035727 | 38037180 | 156735 |
| 50517 | 38088267 | 38090454 | 085788 |
| 50518 | 38125216 | 38127055 | 147535, 085788 |
| 50519 | 38135650 | 38136170 | 147548 |
| 50520 | 38171880 | 38172428 | 147548 |
| 50521 | 38238917 | 38241219 | 272092, 147548 |
| 50522 | 38243462 | 38244890 | 165046 |
| 50523 | 38259012 | 38259985 | 254981, 165046 |
| 50524 | 38264073 | 38265526 | 165046 |
| 50525 | 38266185 | 38267320 | 165046, 272159 |
| 50526 | 38383873 | 38387230 | 196166 |
| 50527 | 38578442 | 38579362 | |
| 50528 | 38585415 | 38586674 | 147526 |
| 50529 | 38591663 | 38593819 | 147526 |
| 50530 | 38598722 | 38600350 | 147526 |
| 50531 | 38613985 | 38615452 | 147526 |
| 50532 | 38624239 | 38624808 | 147526 |
| 50533 | 38644094 | 38645575 | 147526 |
| 50534 | 38650536 | 38651840 | 147526 |
| 50535 | 38749327 | 38750036 | |
| 50536 | 38757989 | 38759742 | 169499 |
| 50537 | 38781891 | 38784086 | 169499 |
| 50538 | 38792810 | 38796382 | 169499 |
| 50539 | 38853149 | 38857649 | 168615, 169490 |
| 50540 | 38894830 | 38897042 | 168615 |
| 50541 | 38899363 | 38900074 | 168615 |
| 50542 | 39809178 | 39810031 | 188676, 253939 |
| 50543 | 39899094 | 39899817 | |
| 50544 | 39911659 | 39913657 | |
| 50545 | 39916763 | 39918131 | |
| 50546 | 39976305 | 39977462 | 253665 |
| 50547 | 39977583 | 39978361 | 253665 |
| 50548 | 40009767 | 40014985 | 176907 |
| 50549 | 40015813 | 40017398 | |
| 50550 | 40019797 | 40020354 | 254143 |
| 50551 | 40028866 | 40033861 | 254143 |
| 50552 | 40230506 | 40231485 | 253354 |
| 50553 | 40304727 | 40305612 | |
| 50554 | 41347111 | 41349054 | 147533 |
| 50555 | 41365410 | 41366432 | 147533 |
| 50556 | 41386085 | 41387445 | 147536 |
| 50557 | 41419278 | 41420786 | 253133 |
| 50558 | 41426837 | 41427670 | 253133 |
| 50559 | 41434881 | 41436774 | 253133, 158669 |
| 50560 | 41461602 | 41463017 | 158669 |
| 50561 | 41467496 | 41468126 | 264578, 158669 |
| 50562 | 41542856 | 41543265 | 029534 |
| 50563 | 41685635 | 41686853 | 260588, 029534 |
| 50564 | 41862693 | 41863275 | 083168 |
| 50565 | 41908227 | 41910618 | 083168 |
| 50566 | 41934830 | 41936076 | |
| 50567 | 42009507 | 42011290 | 070718, 261449 |
| 50568 | 42127886 | 42130065 | 104365, 253408 |
| 50569 | 42173488 | 42175869 | 104365 |
| 50570 | 42183108 | 42184842 | 104365 |
| 50571 | 42195316 | 42196785 | 070501 |
| 50572 | 42223763 | 42224637 | 070501 |
| 50573 | 42248573 | 42250308 | 078668 |
| 50574 | 42330429 | 42331437 | 168575 |
| 50575 | 42333573 | 42335407 | 168575 |
| 50576 | 42342025 | 42342910 | 254131, 168575 |
| 50577 | 42354112 | 42355215 | 168575 |
| 50578 | 42365042 | 42365498 | 168575 |
| 50579 | 42372782 | 42374396 | 168575 |
| 50580 | 42381001 | 42381548 | 168575 |
| 50581 | 42395531 | 42400693 | 176209, 168575 |
| 50582 | 42450944 | 42451681 | |
| 50583 | 42669339 | 42669753 | |
| 50584 | 42697175 | 42698901 | 131931 |
| 50585 | 42750475 | 42753332 | 168172, 266044, 120925 |
| 50586 | 42769793 | 42770738 | 168172 |
| 50587 | 42910510 | 42912896 | 168522, 254673 |
| 50588 | 42947731 | 42949583 | 185900 |
| 50589 | 42979829 | 42980451 | |
| 50590 | 42994680 | 42997440 | 165102 |
| 50591 | 43046348 | 43046885 | 165102 |
| 50592 | 48100067 | 48101125 | 254348, 248347 |
| 50593 | 48172772 | 48174137 | 164808 |
| 50594 | 48227482 | 48228436 | 164808 |
| 50595 | 48252791 | 48253886 | 164808 |
| 50596 | 48264908 | 48267541 | 164808 |
| 50597 | 48271060 | 48272353 | 164808 |
| 50598 | 48281730 | 48282744 | 164808 |
| 50599 | 48343712 | 48344560 | 164808 |
| 50600 | 48384596 | 48385860 | 164808 |
| 50601 | 48410611 | 48412295 | 164808 |
| 50602 | 48424261 | 48427722 | 253330, 164808 |
| 50603 | 48434807 | 48438524 | 164808 |
| 50604 | 48438568 | 48439601 | 164808 |
| 50605 | 48531245 | 48532615 | 164808 |
| 50606 | 48556371 | 48557352 | 164808 |
| 50607 | 48564426 | 48566606 | 164808 |
| 50608 | 48570795 | 48573635 | 164808 |
| 50609 | 48580802 | 48582127 | 164808 |
| 50610 | 48646839 | 48651915 | 221869, 164808 |
| 50611 | 48872021 | 48873118 | 104738, 253729 |
| 50612 | 48873262 | 48874281 | 104738 |
| 50613 | 48905306 | 48906010 | |
| 50614 | 48920198 | 48921894 | 169139 |
| 50615 | 48979624 | 48980211 | |
| 50616 | 48987440 | 48988279 | |
| 50617 | 49059195 | 49060269 | |
| 50618 | 49062029 | 49064871 | |
| 50619 | 49087859 | 49089314 | |
| 50620 | 49308655 | 49309313 | |
| 50621 | 49406294 | 49406746 | |
| 50622 | 49426338 | 49428007 | |
| 50623 | 49549268 | 49550780 | 253608 |
| 50624 | 49561802 | 49562755 | 253608 |
| 50625 | 49647910 | 49648879 | |
| 50626 | 49686135 | 49686975 | |
| 50627 | 49700065 | 49702901 | |
| 50628 | 49728395 | 49729119 | |
| 50629 | 49730210 | 49732887 | |
| 50630 | 49811669 | 49812603 | |
| 50631 | 49814417 | 49815885 | |
| 50632 | 49830992 | 49835125 | 019549 |
| 50633 | 50754725 | 50755344 | |
| 50634 | 50822049 | 50822947 | 147481 |
| 50635 | 50869495 | 50870087 | 147481 |
| 50636 | 50892525 | 50894248 | 147481 |
| 50637 | 50924247 | 50924706 | 147481 |
| 50638 | 51052153 | 51052764 | 147481 |
| 50639 | 51350892 | 51351580 | 147481 |
| 50640 | 51483711 | 51484303 | 147481 |
| 50641 | 51578359 | 51579458 | 147481 |
| 50642 | 51593741 | 51597044 | 147481 |
| 50643 | 52809643 | 52813340 | 228801, 168300 |
| 50644 | 52874409 | 52875725 | 253844 |
| 50645 | 53293960 | 53294445 | 147488 |
| 50646 | 53302348 | 53303967 | 147488 |
| 50647 | 53541464 | 53542416 | 023287 |
| 50648 | 53606441 | 53609131 | 023287 |
| 50649 | 53625598 | 53628316 | 023287 |
| 50650 | 53676234 | 53677737 | |
| 50651 | 53732553 | 53734009 | |
| 50652 | 53752308 | 53752884 | |
| 50653 | 54570132 | 54570904 | |
| 50654 | 54640071 | 54640779 | 047249 |
| 50655 | 54644730 | 54645548 | 047249 |
| 50656 | 54754612 | 54756685 | 047249 |
| 50657 | 54857248 | 54858543 | 147509 |
| 50658 | 54888420 | 54889038 | 187735 |
| 50659 | 54932105 | 54935676 | 187735 |
| 50660 | 55013068 | 55015171 | 120992 |
| 50661 | 55046627 | 55049051 | 137547 |
| 50662 | 55058293 | 55058857 | 137547 |
| 50663 | 55078227 | 55079111 | |
| 50664 | 55084024 | 55084749 | |
| 50665 | 55290792 | 55291256 | |
| 50666 | 55305131 | 55305727 | |
| 50667 | 55334836 | 55335408 | |
| 50668 | 56684593 | 56687209 | 137574, 167904 |
| 50669 | 56791774 | 56793503 | 254087 |
| 50670 | 56805501 | 56806338 | 254325, 254087 |
| 50671 | 56850664 | 56851485 | 254087 |
| 50672 | 56857558 | 56859123 | 254087 |
| 50673 | 56859789 | 56861987 | 254087 |
| 50674 | 56900529 | 56902710 | 254087 |
| 50675 | 56985956 | 56987900 | 238650, 253603, 008988 |
| 50676 | 57123256 | 57124972 | 170791, 181690 |
| 50677 | 57904797 | 57907547 | 104331 |
| 50678 | 58456688 | 58457483 | 253821 |
| 50679 | 58462871 | 58463399 | 253821 |
| 50680 | 58475917 | 58478025 | 253821 |
| 50681 | 58492442 | 58495000 | 253821 |
| 50682 | 58500766 | 58501467 | 253821 |
| 50683 | 58563023 | 58563729 | 253322 |
| 50684 | 58615387 | 58617051 | 253322 |
| 50685 | 58652726 | 58654718 | 253322 |
| 50686 | 58714610 | 58716003 | 253322 |
| 50687 | 58716491 | 58717573 | 253322 |
| 50688 | 58767807 | 58768560 | |
| 50689 | 58773119 | 58774098 | |
| 50690 | 58900703 | 58901544 | |
| 50691 | 58906798 | 58908596 | 169122 |
| 50692 | 58928356 | 58930729 | 169122 |
| 50693 | 58944047 | 58945110 | 169122, 253523 |
| 50694 | 58969990 | 58971286 | 169122 |
| 50695 | 58978329 | 58981815 | 169122 |
| 50696 | 58991339 | 58992447 | 169122 |
| 50697 | 58999256 | 59000674 | 169122 |
| 50698 | 59290708 | 59293305 | |
| 50699 | 59323490 | 59324944 | 215114 |
| 50700 | 59333844 | 59334699 | 215114 |
| 50701 | 59385190 | 59385646 | |
| 50702 | 59390301 | 59391549 | |
| 50703 | 59392792 | 59393363 | |
| 50704 | 59398029 | 59399093 | |
| 50705 | 59465443 | 59467231 | 137575 |
| 50706 | 59469905 | 59470527 | 137575 |
| 50707 | 59476214 | 59476818 | 137575 |
| 50708 | 59571162 | 59573230 | 035681 |
| 50709 | 59754514 | 59755259 | 198846 |
| 50710 | 59942805 | 59943666 | 198846 |
| 50711 | 59946406 | 59947057 | 198846 |
| 50712 | 60973616 | 60974548 | 254775 |
| 50713 | 61393263 | 61394101 | 251396 |
| 50714 | 61405733 | 61406127 | 251396 |
| 50715 | 61428572 | 61431024 | 104388, 251396 |
| 50716 | 61453417 | 61453949 | 104388 |
| 50717 | 61563590 | 61565447 | 254869 |
| 50718 | 61565974 | 61566778 | 254869 |
| 50719 | 61590119 | 61593773 | 171316 |
| 50720 | 61625969 | 61627859 | 171316 |
| 50721 | 61644518 | 61645169 | 171316 |
| 50722 | 61656336 | 61657135 | 171316 |
| 50723 | 61708271 | 61709143 | 171316 |
| 50724 | 61858087 | 61858642 | 254777 |
| 50725 | 61861562 | 61863008 | 254777 |
| 50726 | 61864820 | 61865856 | 254777 |
| 50727 | 61873377 | 61874704 | 254777 |
| 50728 | 61896092 | 61896683 | |
| 50729 | 61897537 | 61898424 | |
| 50730 | 61935080 | 61936885 | |
| 50731 | 62011028 | 62012689 | 177182 |
| 50732 | 62220128 | 62225823 | 177182 |
| 50733 | 62381150 | 62381919 | 177182 |
| 50734 | 62408601 | 62409159 | 177182 |
| 50735 | 62623242 | 62628485 | 264408, 198363 |
| 50736 | 62633288 | 62635209 | |
| 50737 | 62671705 | 62674199 | 251127 |
| 50738 | 62678298 | 62680890 | 251127 |
| 50739 | 62695083 | 62696431 | |
| 50740 | 62698420 | 62699403 | 254119 |
| 50741 | 63009571 | 63010358 | |
| 50742 | 63435710 | 63436845 | 185942 |
| 50743 | 63441724 | 63443155 | 185942 |
| 50744 | 63920220 | 63923688 | |
| 50745 | 63923988 | 63925009 | |
| 50746 | 63927817 | 63929060 | 137563 |
| 50747 | 63936995 | 63939331 | 253121, 137563 |
| 50748 | 63950917 | 63952736 | 137563 |
| 50749 | 63996572 | 63999516 | 137561 |
| 50750 | 64004792 | 64005747 | |
| 50751 | 64080023 | 64082846 | 185728, 270673 |
| 50752 | 64112263 | 64113052 | 185728 |
| 50753 | 64132381 | 64133842 | |
| 50754 | 64534134 | 64535028 | |
| 50755 | 65351930 | 65352766 | |
| 50756 | 65610391 | 65611685 | 172817 |
| 50757 | 65668473 | 65670629 | 172817 |
| 50758 | 65710640 | 65712213 | 172817 |
| 50759 | 65876832 | 65877999 | |
| 50760 | 66064015 | 66065635 | |
| 50761 | 66095225 | 66096191 | |
| 50762 | 66097802 | 66098443 | |
| 50763 | 66545562 | 66548038 | 104442 |
| 50764 | 66555611 | 66557847 | 066855 |
| 50765 | 66629257 | 66630301 | 205268, 066855 |
| 50766 | 66685261 | 66685771 | 205268 |
| 50767 | 66718593 | 66719473 | 205268 |
| 50768 | 66752973 | 66755557 | 272192, 205268 |
| 50769 | 66938638 | 66939253 | 147570 |
| 50770 | 67002756 | 67004916 | 147570 |
| 50771 | 67024343 | 67028046 | 253190 |
| 50772 | 67035174 | 67038079 | |
| 50773 | 67042003 | 67042781 | 147573 |
| 50774 | 67064253 | 67064637 | 147573 |
| 50775 | 67088687 | 67090106 | 147571 |
| 50776 | 67227877 | 67228840 | |
| 50777 | 67340134 | 67342999 | 147576, 179041, 246145 |
| 50778 | 67343885 | 67345985 | 147576 |
| 50779 | 67374362 | 67377029 | 147576, 169085 |
| 50780 | 67378848 | 67380797 | 147576, 169085 |
| 50781 | 67382332 | 67385042 | 147576, 169085 |
| 50782 | 67385415 | 67386632 | 169085 |
| 50783 | 67419086 | 67420294 | 169085 |
| 50784 | 67424310 | 67425198 | 169085 |
| 50785 | 67432098 | 67432651 | |
| 50786 | 67433822 | 67435918 | |
| 50787 | 67446776 | 67449173 | |
| 50788 | 67525058 | 67526868 | 185697 |
| 50789 | 67578507 | 67580687 | 213865, 270024, 175073 |
| 50790 | 67624158 | 67625740 | 104205, 270024 |
| 50791 | 67687051 | 67687929 | 270024, 104205 |
| 50792 | 67836400 | 67838410 | 245910 |
| 50793 | 67973358 | 67975610 | 104218, 121022 |
| 50794 | 67976255 | 67977624 | 121022, 104218 |
| 50795 | 68084997 | 68085520 | 104218 |
| 50796 | 68145601 | 68147311 | 066777 |
| 50797 | 68254872 | 68256937 | 271966, 066777 |
| 50798 | 69560596 | 69561042 | 165084 |
| 50799 | 69643617 | 69644904 | 165084 |
| 50800 | 69645155 | 69647509 | 165084 |
| 50801 | 70032715 | 70033402 | |
| 50802 | 70506613 | 70507374 | 137573 |
| 50803 | 70517040 | 70518338 | 137573 |
| 50804 | 70637853 | 70638523 | 137571 |
| 50805 | 70745123 | 70747444 | 246528, 137571 |
| 50806 | 71000509 | 71001367 | |
| 50807 | 71018775 | 71019595 | |
| 50808 | 71060074 | 71061080 | 140396 |
| 50809 | 71064564 | 71065335 | 140396 |
| 50810 | 71092793 | 71093341 | 140396 |
| 50811 | 71105783 | 71107439 | 140396 |
| 50812 | 71242687 | 71245961 | 140396 |
| 50813 | 71313792 | 71317218 | 140396 |
| 50814 | 71369201 | 71369976 | |
| 50815 | 71518694 | 71525850 | 246366, 067167 |
| 50816 | 71577820 | 71581842 | 221947, 147592 |
| 50817 | 71682842 | 71686517 | 221947 |
| 50818 | 71959185 | 71959861 | |
| 50819 | 71971036 | 71971911 | |
| 50820 | 72023930 | 72024888 | |
| 50821 | 72042384 | 72043862 | |
| 50822 | 72741991 | 72746484 | 264576, 235531 |
| 50823 | 72754238 | 72755918 | 178860, 235531 |
| 50824 | 73315787 | 73316306 | |
| 50825 | 73920780 | 73921937 | 147601 |
| 50826 | 74086313 | 74087027 | |
| 50827 | 74204786 | 74208729 | 121039, 147604 |
| 50828 | 74209010 | 74209870 | 121039 |
| 50829 | 74253311 | 74253749 | 250295 |
| 50830 | 74270489 | 74271977 | 253339 |
| 50831 | 74276345 | 74277671 | |
| 50832 | 74320388 | 74320972 | |
| 50833 | 74332116 | 74332719 | 040341, 253302 |
| 50834 | 74456738 | 74457642 | 040341 |
| 50835 | 74473084 | 74473807 | 040341 |
| 50836 | 74658018 | 74660111 | 040341, 258677 |
| 50837 | 74789759 | 74792911 | 104343, 258677 |
| 50838 | 74883215 | 74885858 | 175606, 154582 |
| 50839 | 74888024 | 74889402 | 175606 |
| 50840 | 75080145 | 75080889 | |
| 50841 | 75086138 | 75086613 | |
| 50842 | 75179124 | 75180366 | 104369 |
| 50843 | 75189355 | 75189982 | 104369 |
| 50844 | 75262340 | 75263206 | 104381 |
| 50845 | 75318330 | 75318858 | 104381 |
| 50846 | 75547693 | 75548332 | 254349 |
| 50847 | 75572196 | 75573000 | 254349 |
| 50848 | 75648396 | 75649087 | 254349 |
| 50849 | 75825623 | 75826238 | |
| 50850 | 76178788 | 76179726 | 249395 |
| 50851 | 76242498 | 76243544 | |
| 50852 | 76305871 | 76306814 | |
| 50853 | 76307530 | 76309017 | |
| 50854 | 76316038 | 76317524 | |
| 50855 | 76318341 | 76321753 | 164749 |
| 50856 | 76370515 | 76371434 | 164749 |
| 50857 | 76417312 | 76418341 | 164749 |
| 50858 | 77585016 | 77587527 | 253661 |
| 50859 | 77589344 | 77590998 | 253661 |
| 50860 | 77592566 | 77596618 | 091656, 253661 |
| 50861 | 77631427 | 77631837 | 091656 |
| 50862 | 77724336 | 77724780 | 091656 |
| 50863 | 77890260 | 77891199 | |
| 50864 | 77911456 | 77913580 | 164751 |
| 50865 | 78311201 | 78312735 | 254366 |
| 50866 | 78319440 | 78320022 | 254366 |
| 50867 | 78519537 | 78520092 | |
| 50868 | 79715734 | 79717868 | 104432 |
| 50869 | 79746648 | 79747069 | |
| 50870 | 79761319 | 79763113 | |
| 50871 | 79932892 | 79933854 | |
| 50872 | 79975751 | 79976387 | |
| 50873 | 79985530 | 79986771 | |
| 50874 | 80679090 | 80681799 | 249328, 272138, 164683 |
| 50875 | 80695679 | 80697035 | 249328, 272138 |
| 50876 | 80705384 | 80707391 | 249328, 272138 |
| 50877 | 80742173 | 80743130 | 249328 |
| 50878 | 80802495 | 80804597 | |
| 50879 | 80941126 | 80943005 | 147586, 076554 |
| 50880 | 80995678 | 80998544 | 076554 |
| 50881 | 81003823 | 81004338 | 076554 |
| 50882 | 81019417 | 81020045 | 076554 |
| 50883 | 81061676 | 81062231 | 076554 |
| 50884 | 81082698 | 81084208 | 076554 |
| 50885 | 81119110 | 81119635 | 076554 |
| 50886 | 81152581 | 81153764 | 265588 |
| 50887 | 81168964 | 81170976 | |
| 50888 | 81210277 | 81210876 | 253238 |
| 50889 | 81227221 | 81227794 | 253238 |
| 50890 | 81278977 | 81280025 | |
| 50891 | 81347595 | 81348734 | |
| 50892 | 81397360 | 81400705 | 205189, 251867 |
| 50893 | 81477754 | 81479369 | |
| 50894 | 81488947 | 81492477 | |
| 50895 | 81589753 | 81590346 | 164684 |
| 50896 | 81639533 | 81640330 | 164684 |
| 50897 | 81786529 | 81787366 | 164684 |
| 50898 | 81820411 | 81821738 | |
| 50899 | 81830143 | 81831613 | |
| 50900 | 81837424 | 81839994 | |
| 50901 | 81849541 | 81850115 | |
| 50902 | 81876877 | 81878310 | |
| 50903 | 81903876 | 81904761 | 076641 |
| 50904 | 81940707 | 81942505 | 076641 |
| 50905 | 81945425 | 81946712 | 076641 |
| 50906 | 81962149 | 81964184 | 076641 |
| 50907 | 82023352 | 82024759 | 076641 |
| 50908 | 82069120 | 82070040 | 253214 |
| 50909 | 82105327 | 82106875 | |
| 50910 | 82123700 | 82124756 | |
| 50911 | 82178637 | 82179164 | |
| 50912 | 82192083 | 82193165 | 164687, 254027 |
| 50913 | 82469733 | 82470657 | |
| 50914 | 82597772 | 82598796 | 133731 |
| 50915 | 82606112 | 82608224 | 253598 |
| 50916 | 82632297 | 82634098 | 104231 |
| 50917 | 82643879 | 82645682 | 164695, 104231 |
| 50918 | 82753754 | 82755372 | 104497 |
| 50919 | 82770617 | 82771506 | |
| 50920 | 85169677 | 85170112 | 184672 |
| 50921 | 85201176 | 85201845 | 184672 |
| 50922 | 85206641 | 85207287 | 184672 |
| 50923 | 85391345 | 85393710 | 184672 |
| 50924 | 85514109 | 85514538 | 184672 |
| 50925 | 86019178 | 86019982 | 133739 |
| 50926 | 86088881 | 86090832 | 254208, 133740, 260493 |
| 50927 | 86100497 | 86101112 | 133740 |
| 50928 | 86131989 | 86134634 | 185015, 176731 |
| 50929 | 86157043 | 86158775 | 257962, 258256, 185015 |
| 50930 | 86322770 | 86323355 | 164879 |
| 50931 | 86375300 | 86377044 | 104267, 253549 |
| 50932 | 86447235 | 86448760 | 254380 |
| 50933 | 86458274 | 86460206 | |
| 50934 | 86848073 | 86848662 | |
| 50935 | 86937107 | 86938216 | 253154 |
| 50936 | 87130531 | 87131653 | 147614, 253675 |
| 50937 | 87267595 | 87268748 | 164893 |
| 50938 | 87302173 | 87303441 | 164893 |
| 50939 | 87331914 | 87334196 | 164893 |
| 50940 | 87353901 | 87357001 | 123124, 254231 |
| 50941 | 87426442 | 87427167 | 123124 |
| 50942 | 87496094 | 87496731 | 176623 |
| 50943 | 87519990 | 87521405 | 085719, 176623 |
| 50944 | 87526230 | 87527660 | 176623, 085719 |
| 50945 | 87715477 | 87716028 | 170289 |
| 50946 | 87840111 | 87841198 | |
| 50947 | 90732590 | 90733588 | 251136 |
| 50948 | 90741094 | 90741651 | 251136 |
| 50949 | 90742011 | 90742710 | 251136 |
| 50950 | 90769104 | 90772458 | 104312, 251136 |
| 50951 | 90808413 | 90814832 | |
| 50952 | 90894450 | 90895363 | |
| 50953 | 90898055 | 90899016 | |
| 50954 | 90900915 | 90902774 | |
| 50955 | 90913674 | 90916017 | 164823 |
| 50956 | 90995505 | 90997217 | 104320 |
| 50957 | 91012387 | 91014993 | 104325, 104320 |
| 50958 | 91020533 | 91021555 | 104325 |
| 50959 | 91024787 | 91025350 | 104325 |
| 50960 | 91479190 | 91480770 | 253394 |
| 50961 | 91589949 | 91590781 | 254180 |
| 50962 | 91655486 | 91656211 | 180694 |
| 50963 | 91656777 | 91659080 | 246792, 180694 |
| 50964 | 91842204 | 91843106 | 254251, 123119 |
| 50965 | 91889161 | 91889694 | 123119 |
| 50966 | 91899871 | 91901821 | 123119 |
| 50967 | 92052922 | 92053472 | 155099 |
| 50968 | 92081546 | 92083298 | 155100, 253738 |
| 50969 | 93976601 | 93978835 | 253197, 205133 |
| 50970 | 94039144 | 94039934 | |
| 50971 | 94341301 | 94343465 | 246662 |
| 50972 | 94364861 | 94365963 | 246662 |
| 50973 | 94547130 | 94548995 | 246662 |
| 50974 | 94576662 | 94577940 | 246662 |
| 50975 | 94590403 | 94593018 | 246662 |
| 50976 | 94593281 | 94595609 | 246662 |
| 50977 | 94605985 | 94607682 | 246662 |
| 50978 | 94667587 | 94668197 | 246662 |
| 50979 | 94678150 | 94679228 | 246662 |
| 50980 | 94706025 | 94706783 | 246662 |
| 50981 | 94710694 | 94711944 | 188343, 246662 |
| 50982 | 94712285 | 94713502 | 246662, 188343 |
| 50983 | 94751915 | 94753522 | 253848, 212998, 183808 |
| 50984 | 94760820 | 94761377 | |
| 50985 | 94766555 | 94767633 | 164953 |
| 50986 | 94886009 | 94886698 | 164951 |
| 50987 | 94928520 | 94929927 | 264448, 164951 |
| 50988 | 94948088 | 94948695 | |
| 50989 | 95231170 | 95232173 | |
| 50990 | 95252742 | 95255729 | |
| 50991 | 95270492 | 95275422 | 164949 |
| 50992 | 95429671 | 95430403 | 265817, 197275 |
| 50993 | 95534659 | 95535226 | 164944 |
| 50994 | 95564781 | 95566944 | 253704, 164944 |
| 50995 | 95731284 | 95733179 | 156162 |
| 50996 | 95835303 | 95836449 | 164941 |
| 50997 | 95907306 | 95908250 | 156170, 175305 |
| 50998 | 95908485 | 95909317 | 175305, 156170 |
| 50999 | 95917416 | 95919211 | 238791, 156170 |
| 51000 | 95958933 | 95964907 | 253878, 164938, 156170 |
| 51001 | 95971117 | 95974774 | 156170 |
| 51002 | 95975235 | 95976516 | 156170 |
| 51003 | 95979397 | 95982073 | 156170 |
| 51004 | 96000229 | 96000885 | 156170 |
| 51005 | 96022787 | 96025487 | 156170 |
| 51006 | 96032600 | 96033521 | 156170 |
| 51007 | 96036229 | 96038494 | 156170 |
| 51008 | 96076560 | 96080331 | 245080, 156170 |
| 51009 | 96081058 | 96081579 | 156170, 245080 |
| 51010 | 96083502 | 96084416 | 254248, 156170, 245080 |
| 51011 | 96086641 | 96087710 | 156170, 254248 |
| 51012 | 96111381 | 96115365 | 156170 |
| 51013 | 96124197 | 96125969 | 156170 |
| 51014 | 96145534 | 96147548 | 175895 |
| 51015 | 96181712 | 96182793 | |
| 51016 | 96186744 | 96187709 | |
| 51017 | 96212389 | 96213101 | |
| 51018 | 96237446 | 96238805 | 253773 |
| 51019 | 96280517 | 96282951 | 156172, 253773 |
| 51020 | 96690559 | 96691379 | 223297, 253773 |
| 51021 | 96705293 | 96706117 | 253773 |
| 51022 | 96714310 | 96715571 | 253773 |
| 51023 | 96804942 | 96806084 | 253773 |
| 51024 | 97246845 | 97248117 | 254224, 156467 |
| 51025 | 97272881 | 97275246 | 156471, 156469 |
| 51026 | 97280374 | 97281324 | 156471 |
| 51027 | 97351353 | 97352015 | |
| 51028 | 97353750 | 97354212 | |
| 51029 | 97365152 | 97365887 | |
| 51030 | 97455804 | 97457022 | |
| 51031 | 97460763 | 97461802 | |
| 51032 | 97505351 | 97509508 | 169439 |
| 51033 | 97526162 | 97527302 | 169439 |
| 51034 | 97535938 | 97537135 | 169439 |
| 51035 | 97582300 | 97583966 | 169439 |
| 51036 | 97601027 | 97604902 | 169439 |
| 51037 | 97612252 | 97613628 | 169439 |
| 51038 | 97629136 | 97629781 | |
| 51039 | 97630255 | 97630885 | |
| 51040 | 97656646 | 97659052 | 104324 |
| 51041 | 97659385 | 97659750 | 104324 |
| 51042 | 97743456 | 97744573 | 104324 |
| 51043 | 97801488 | 97806195 | 104324 |
| 51044 | 97841105 | 97842712 | 104324 |
| 51045 | 97845127 | 97845979 | 104324 |
| 51046 | 97853699 | 97854166 | 104324 |
| 51047 | 97861340 | 97862000 | 104324 |
| 51048 | 97864398 | 97866267 | 104324 |
| 51049 | 97940570 | 97941172 | 104324 |
| 51050 | 98073564 | 98075017 | 104324 |
| 51051 | 98076265 | 98078861 | 104324 |
| 51052 | 98102280 | 98103952 | 104324 |
| 51053 | 98126900 | 98128058 | 104324 |
| 51054 | 98130292 | 98131261 | 104324 |
| 51055 | 98147004 | 98147497 | 104324 |
| 51056 | 98160181 | 98161440 | 104324 |
| 51057 | 98175400 | 98176620 | |
| 51058 | 98289871 | 98290262 | 180543 |
| 51059 | 98336615 | 98337546 | |
| 51060 | 98375619 | 98376372 | |
| 51061 | 98556122 | 98557652 | |
| 51062 | 98564898 | 98566782 | |
| 51063 | 98572108 | 98572848 | |
| 51064 | 98630490 | 98630948 | |
| 51065 | 98655583 | 98658640 | 147649 |
| 51066 | 98787233 | 98788660 | 104341 |
| 51067 | 98830356 | 98831102 | 104341 |
| 51068 | 98861418 | 98862237 | 104341 |
| 51069 | 98880600 | 98882505 | 132561 |
| 51070 | 98917259 | 98918331 | 132561 |
| 51071 | 98927173 | 98928456 | 132561 |
| 51072 | 98929378 | 98931809 | 132561 |
| 51073 | 98937483 | 98939396 | 132561 |
| 51074 | 98940545 | 98942338 | 132561 |
| 51075 | 98945558 | 98946673 | 132561 |
| 51076 | 99056426 | 99059150 | 156482 |
| 51077 | 99080370 | 99082514 | 177459 |
| 51078 | 99083491 | 99084005 | 177459 |
| 51079 | 99103407 | 99104149 | 177459 |
| 51080 | 99108632 | 99109452 | |
| 51081 | 99120292 | 99120851 | 132541 |
| 51082 | 99124447 | 99126153 | 132541 |
| 51083 | 99128704 | 99130758 | 104356, 132541 |
| 51084 | 99305308 | 99307155 | 104361 |
| 51085 | 99309866 | 99310541 | |
| 51086 | 99449433 | 99450261 | 104375, 272321 |
| 51087 | 99737898 | 99738633 | 104375 |
| 51088 | 99837229 | 99838105 | 104375 |
| 51089 | 100024480 | 100026384 | 132549, 253948 |
| 51090 | 100318202 | 100319371 | 132549 |
| 51091 | 100332838 | 100333526 | 132549 |
| 51092 | 100510805 | 100511388 | 132549 |
| 51093 | 100649483 | 100649950 | 132549 |
| 51094 | 100694653 | 100695091 | 132549 |
| 51095 | 100709890 | 100710327 | 235683, 132549 |
| 51096 | 100717471 | 100719382 | 132549 |
| 51097 | 100786940 | 100787638 | 132549 |
| 51098 | 100886135 | 100886709 | 164919, 132549 |
| 51099 | 100904599 | 100906292 | 164919 |
| 51100 | 101162201 | 101163774 | 147669 |
| 51101 | 101169892 | 101171019 | 104450 |
| 51102 | 101175555 | 101176312 | 104450 |
| 51103 | 101224933 | 101226123 | 104450 |
| 51104 | 101314971 | 101316557 | 034677 |
| 51105 | 101319533 | 101323330 | 034677 |
| 51106 | 101371398 | 101374166 | |
| 51107 | 101456072 | 101456522 | |
| 51108 | 101469356 | 101474447 | |
| 51109 | 101474498 | 101478880 | |
| 51110 | 101478929 | 101481410 | |
| 51111 | 101483702 | 101489859 | 253666 |
| 51112 | 101493911 | 101495789 | 253666 |
| 51113 | 101498908 | 101501218 | 253666 |
| 51114 | 101501731 | 101505310 | 254364, 253666 |
| 51115 | 101555986 | 101556675 | 186106 |
| 51116 | 101570835 | 101572452 | 186106 |
| 51117 | 101675119 | 101676368 | 174226 |
| 51118 | 101732637 | 101735612 | 070756 |
| 51119 | 101836772 | 101837833 | |
| 51120 | 101880562 | 101882398 | |
| 51121 | 101911618 | 101913578 | |
| 51122 | 101961938 | 101966526 | 164924 |
| 51123 | 102063443 | 102064650 | 248599 |
| 51124 | 102110654 | 102111106 | |
| 51125 | 102137881 | 102140090 | 253395 |
| 51126 | 102166619 | 102168524 | |
| 51127 | 102170003 | 102172903 | |
| 51128 | 102180423 | 102180921 | 261087 |
| 51129 | 102216031 | 102220451 | 271882, 120963 |
| 51130 | 102299590 | 102300712 | 253282 |
| 51131 | 102371257 | 102372069 | |
| 51132 | 102372511 | 102373929 | |
| 51133 | 102387493 | 102389061 | |
| 51134 | 102462429 | 102468053 | 254084 |
| 51135 | 102478880 | 102480342 | 254024 |
| 51136 | 102594906 | 102597589 | 083307 |
| 51137 | 102598695 | 102600938 | 083307 |
| 51138 | 102647426 | 102648415 | 083307 |
| 51139 | 102769040 | 102770021 | 104490 |
| 51140 | 102979783 | 102980328 | 104490 |
| 51141 | 103248086 | 103252359 | 246263, 048392 |
| 51142 | 103343265 | 103343881 | 104517 |
| 51143 | 103415771 | 103416171 | 104517 |
| 51144 | 103422917 | 103425739 | 104517 |
| 51145 | 103596096 | 103598394 | |
| 51146 | 103612628 | 103614321 | |
| 51147 | 103625749 | 103626415 | |
| 51148 | 103640658 | 103641447 | |
| 51149 | 103665517 | 103670009 | 155090 |
| 51150 | 103697571 | 103698640 | |
| 51151 | 103714553 | 103715357 | |
| 51152 | 103781982 | 103783176 | |
| 51153 | 103788559 | 103789470 | |
| 51154 | 103794641 | 103795839 | |
| 51155 | 103799680 | 103803436 | |
| 51156 | 103809526 | 103810565 | |
| 51157 | 103816470 | 103821863 | 253669 |
| 51158 | 103822003 | 103823885 | 253669 |
| 51159 | 103874630 | 103877848 | 253320, 155096 |
| 51160 | 103906382 | 103907337 | 253320 |
| 51161 | 103914911 | 103916481 | 253320 |
| 51162 | 103918010 | 103919707 | 253320 |
| 51163 | 103921753 | 103923277 | 253320 |
| 51164 | 103929545 | 103930541 | 253320 |
| 51165 | 103930616 | 103933818 | 253320 |
| 51166 | 103933965 | 103934618 | 253320 |
| 51167 | 104032497 | 104034264 | 155097, 254236 |
| 51168 | 104081992 | 104082776 | 155097 |
| 51169 | 104219486 | 104222162 | 164929, 247081 |
| 51170 | 104282345 | 104283440 | 247081 |
| 51171 | 104310652 | 104311728 | 164930, 247081 |
| 51172 | 104426313 | 104428695 | 164934, 164933 |
| 51173 | 105270465 | 105271178 | |
| 51174 | 105289496 | 105292095 | |
| 51175 | 105335366 | 105335948 | |
| 51176 | 105337590 | 105338714 | |
| 51177 | 105385354 | 105386235 | 147647 |
| 51178 | 105471236 | 105473246 | 147647 |
| 51179 | 105475773 | 105476689 | 147647 |
| 51180 | 105477776 | 105479621 | 147647 |
| 51181 | 105600800 | 105601867 | 147650 |
| 51182 | 106908885 | 106909813 | 251003 |
| 51183 | 107135195 | 107135970 | |
| 51184 | 107618852 | 107619683 | 253582, 164830 |
| 51185 | 107629798 | 107631472 | 253582, 164830 |
| 51186 | 107635967 | 107638281 | 253582, 164830 |
| 51187 | 107640731 | 107641843 | 253582, 164830 |
| 51188 | 107669413 | 107671172 | 253582, 164830 |
| 51189 | 107875925 | 107877430 | |
| 51190 | 109151805 | 109152427 | |
| 51191 | 109218876 | 109219464 | 104408 |
| 51192 | 109259697 | 109261418 | 104408 |
| 51193 | 109454813 | 109456429 | 104412 |
| 51194 | 109596191 | 109597718 | 253949 |
| 51195 | 109799232 | 109800008 | 164841 |
| 51196 | 109817215 | 109818216 | |
| 51197 | 109873229 | 109874042 | |
| 51198 | 109892795 | 109893610 | |
| 51199 | 110240410 | 110242713 | |
| 51200 | 110345363 | 110347096 | 120533, 120526 |
| 51201 | 110551789 | 110553643 | 147654 |
| 51202 | 110594058 | 110595068 | 147642 |
| 51203 | 110605152 | 110608429 | 147642 |
| 51204 | 110608707 | 110611036 | 147642 |
| 51205 | 110613010 | 110616331 | 147642 |
| 51206 | 110618280 | 110620504 | 147642 |
| 51207 | 110651788 | 110653157 | 147642 |
| 51208 | 110655196 | 110658440 | 248050, 147642 |
| 51209 | 111173118 | 111174247 | |
| 51210 | 111860245 | 111861549 | |
| 51211 | 113469712 | 113470912 | 164796 |
| 51212 | 113471927 | 113472616 | 164796 |
| 51213 | 115981949 | 115983686 | |
| 51214 | 116680345 | 116682027 | 104447 |
| 51215 | 116786506 | 116787609 | 104447 |
| 51216 | 116973803 | 116974894 | 249917 |
| 51217 | 116981545 | 116982279 | 249917 |
| 51218 | 117006747 | 117007358 | 249917 |
| 51219 | 117068749 | 117070988 | 249917 |
| 51220 | 117124692 | 117126355 | 249917 |
| 51221 | 117185719 | 117186414 | 249917 |
| 51222 | 117767134 | 117769025 | 147677 |
| 51223 | 117777651 | 117779506 | 147679, 147677 |
| 51224 | 117885699 | 117887400 | 253327, 264875, 164754 |
| 51225 | 118316229 | 118316816 | |
| 51226 | 118532515 | 118533788 | 164758 |
| 51227 | 118622533 | 118623177 | |
| 51228 | 118627097 | 118628021 | |
| 51229 | 118630974 | 118632502 | |
| 51230 | 118662333 | 118663029 | |
| 51231 | 118675484 | 118676264 | |
| 51232 | 118681365 | 118685499 | |
| 51233 | 118724632 | 118726129 | |
| 51234 | 118878463 | 118880847 | 182197 |
| 51235 | 118897752 | 118899146 | 182197 |
| 51236 | 118918948 | 118919432 | 182197 |
| 51237 | 118922292 | 118922703 | 182197 |
| 51238 | 118938539 | 118941552 | 182197 |
| 51239 | 118946266 | 118947278 | 182197 |
| 51240 | 118949371 | 118950230 | 182197 |
| 51241 | 118958042 | 118960754 | 182197 |
| 51242 | 118989579 | 118991560 | 182197 |
| 51243 | 118992173 | 118993944 | 182197 |
| 51244 | 119000597 | 119002683 | 182197 |
| 51245 | 119012434 | 119013261 | 182197 |
| 51246 | 119016843 | 119018157 | 182197 |
| 51247 | 119020569 | 119025548 | 182197 |
| 51248 | 119029059 | 119032259 | 182197 |
| 51249 | 119037278 | 119037869 | 182197 |
| 51250 | 119041729 | 119042942 | 182197 |
| 51251 | 119067927 | 119069604 | 182197 |
| 51252 | 119115686 | 119116777 | 182197 |
| 51253 | 119119454 | 119125470 | 182197 |
| 51254 | 119202154 | 119204112 | 177570 |
| 51255 | 119207868 | 119208818 | 177570 |
| 51256 | 119356061 | 119357275 | 225885, 177570 |
| 51257 | 119368324 | 119369376 | 225885, 177570 |
| 51258 | 119391683 | 119392716 | 225885, 177570 |
| 51259 | 119393623 | 119396738 | 225885, 177570 |
| 51260 | 119398423 | 119399513 | 225885, 177570 |
| 51261 | 119402550 | 119403273 | 225885, 177570 |
| 51262 | 119451677 | 119452453 | 177570 |
| 51263 | 119480641 | 119481695 | 177570 |
| 51264 | 119516777 | 119518318 | 177570 |
| 51265 | 119518600 | 119519626 | 177570 |
| 51266 | 119529024 | 119529903 | 177570 |
| 51267 | 119558737 | 119559429 | 177570 |
| 51268 | 119633670 | 119634766 | 177570 |
| 51269 | 119915833 | 119917034 | |
| 51270 | 119963625 | 119964998 | 164761 |
| 51271 | 119974054 | 119974660 | |
| 51272 | 120065996 | 120066818 | 184374 |
| 51273 | 120188915 | 120190387 | 147676 |
| 51274 | 120217085 | 120217748 | 147676 |
| 51275 | 120220369 | 120222345 | 147676 |
| 51276 | 120254001 | 120254575 | 147676, 253972 |
| 51277 | 120647818 | 120648488 | 136960 |
| 51278 | 120650922 | 120651861 | 136960 |
| 51279 | 120662326 | 120664089 | 136960 |
| 51280 | 120667057 | 120667865 | 136960 |
| 51281 | 120684272 | 120686292 | 136960 |
| 51282 | 120712975 | 120713407 | |
| 51283 | 120734018 | 120734853 | |
| 51284 | 120779674 | 120780406 | 064313 |
| 51285 | 120844293 | 120845935 | 064313 |
| 51286 | 120856917 | 120857749 | 136982 |
| 51287 | 120867555 | 120869021 | 136982 |
| 51288 | 120885323 | 120887463 | 155792, 245330 |
| 51289 | 120907808 | 120908939 | 155792 |
| 51290 | 120924385 | 120925172 | 155792 |
| 51291 | 120927516 | 120928423 | 155792 |
| 51292 | 120931183 | 120933357 | 155792 |
| 51293 | 121072865 | 121073318 | 187955 |
| 51294 | 121079517 | 121081279 | 187955 |
| 51295 | 121082058 | 121082611 | 187955 |
| 51296 | 121094622 | 121095923 | 187955 |
| 51297 | 121096706 | 121097607 | 187955 |
| 51298 | 121415917 | 121417280 | 172172 |
| 51299 | 121456441 | 121458059 | 172167, 172172 |
| 51300 | 121597486 | 121598143 | 172164 |
| 51301 | 121631365 | 121632458 | 172164 |
| 51302 | 121667612 | 121669413 | 172164 |
| 51303 | 121681052 | 121681526 | 172164 |
| 51304 | 121682682 | 121683680 | 172164 |
| 51305 | 121757451 | 121757864 | 172164 |
| 51306 | 121771528 | 121773070 | 172164 |
| 51307 | 121790150 | 121790900 | 172164 |
| 51308 | 121791677 | 121793697 | 172164 |
| 51309 | 121797912 | 121798343 | 172164 |
| 51310 | 121820590 | 121825449 | 272502, 172164 |
| 51311 | 121827302 | 121828058 | |
| 51312 | 121838589 | 121840998 | |
| 51313 | 121851288 | 121851974 | |
| 51314 | 121857665 | 121859142 | |
| 51315 | 121873510 | 121874076 | |
| 51316 | 122109552 | 122110285 | 253619 |
| 51317 | 122117490 | 122118392 | 253619 |
| 51318 | 122176292 | 122176783 | |
| 51319 | 122252669 | 122253232 | |
| 51320 | 122755168 | 122755677 | |
| 51321 | 122782400 | 122782953 | |
| 51322 | 122940240 | 122941187 | |
| 51323 | 122970320 | 122971355 | 254303 |
| 51324 | 123093897 | 123094948 | 254303 |
| 51325 | 123312336 | 123313044 | |
| 51326 | 123564572 | 123565750 | |
| 51327 | 123580027 | 123582247 | |
| 51328 | 123599941 | 123600297 | |
| 51329 | 123607957 | 123608600 | |
| 51330 | 123671367 | 123671951 | |
| 51331 | 123701324 | 123703762 | 253819 |
| 51332 | 123704888 | 123707329 | 253819 |
| 51333 | 123733869 | 123734535 | |
| 51334 | 123745703 | 123746482 | |
| 51335 | 123769103 | 123769808 | |
| 51336 | 123770304 | 123771098 | |
| 51337 | 123771984 | 123773470 | |
| 51338 | 123791928 | 123795902 | 272043, 178764, 272384 |
| 51339 | 123829100 | 123829657 | 178764 |
| 51340 | 123899496 | 123900948 | 178764 |
| 51341 | 123971144 | 123973417 | 178764 |
| 51342 | 123981694 | 123983378 | 178764 |
| 51343 | 124000327 | 124002014 | |
| 51344 | 124049664 | 124051815 | 136986 |
| 51345 | 124053401 | 124055426 | 156787, 136986 |
| 51346 | 124068973 | 124071316 | 156787 |
| 51347 | 124084470 | 124085714 | 156787 |
| 51348 | 124168765 | 124170533 | |
| 51349 | 124172467 | 124174036 | |
| 51350 | 124179308 | 124180411 | |
| 51351 | 124217006 | 124218020 | 147689 |
| 51352 | 124228766 | 124230353 | |
| 51353 | 124252540 | 124253877 | 189376, 259305 |
| 51354 | 124284483 | 124288094 | 165156 |
| 51355 | 124386387 | 124387502 | 156802 |
| 51356 | 124407641 | 124409266 | 156802 |
| 51357 | 124428198 | 124429655 | 156795, 156802 |
| 51358 | 124465512 | 124466471 | 156795 |
| 51359 | 124528772 | 124530179 | 156804 |
| 51360 | 124539812 | 124541516 | 156804 |
| 51361 | 124552360 | 124553925 | 156804 |
| 51362 | 124593114 | 124593927 | |
| 51363 | 124623951 | 124627045 | 253286 |
| 51364 | 124637433 | 124637839 | 253286 |
| 51365 | 124641370 | 124644005 | 253286 |
| 51366 | 124647404 | 124647875 | 253286 |
| 51367 | 124682872 | 124684302 | |
| 51368 | 124692019 | 124693191 | 104537 |
| 51369 | 124731921 | 124733057 | 104537 |
| 51370 | 124751729 | 124752191 | |
| 51371 | 124780126 | 124782346 | 176853 |
| 51372 | 124907047 | 124907619 | 214814 |
| 51373 | 124934010 | 124935193 | 214814 |
| 51374 | 125029803 | 125030467 | 214814, 181171 |
| 51375 | 125115988 | 125116953 | 214814, 253868 |
| 51376 | 125212204 | 125213011 | 214803 |
| 51377 | 125214071 | 125214996 | 214803 |
| 51378 | 125281833 | 125284804 | 253227 |
| 51379 | 125383517 | 125385779 | 164983 |
| 51380 | 125485078 | 125489027 | 170881, 245149 |
| 51381 | 125550176 | 125552209 | 147684, 147687 |
| 51382 | 125575509 | 125576668 | 147684, 170873 |
| 51383 | 125589935 | 125590503 | 170873 |
| 51384 | 125612789 | 125614429 | 170873 |
| 51385 | 125628354 | 125633202 | 170873 |
| 51386 | 125640309 | 125644481 | 170873 |
| 51387 | 125661204 | 125662237 | 170873 |
| 51388 | 125685569 | 125686343 | 170873 |
| 51389 | 125704560 | 125705271 | 170873 |
| 51390 | 125726225 | 125728443 | 170873 |
| 51391 | 125731963 | 125733741 | 170873 |
| 51392 | 125733978 | 125735618 | 170873 |
| 51393 | 125735709 | 125741481 | 170873 |
| 51394 | 125752800 | 125753522 | |
| 51395 | 125755270 | 125756150 | |
| 51396 | 125763472 | 125764177 | |
| 51397 | 125783593 | 125785274 | |
| 51398 | 125878740 | 125879256 | 249816 |
| 51399 | 125971483 | 125972365 | |
| 51400 | 125973559 | 125974148 | |
| 51401 | 125985043 | 125986400 | 180938 |
| 51402 | 126009501 | 126012091 | 104549, 253513 |
| 51403 | 126043106 | 126045090 | 164961 |
| 51404 | 126103012 | 126104842 | 156831, 164961 |
| 51405 | 126122344 | 126124282 | 156831 |
| 51406 | 126130376 | 126132418 | 156831 |
| 51407 | 126137164 | 126139266 | 156831 |
| 51408 | 126140213 | 126143503 | 156831 |
| 51409 | 126163808 | 126164997 | 156831 |
| 51410 | 126179118 | 126182765 | 156831 |
| 51411 | 126185942 | 126188212 | 156831 |
| 51412 | 126193734 | 126194701 | 156831 |
| 51413 | 126203886 | 126204765 | 156831 |
| 51414 | 126230804 | 126234027 | 156831 |
| 51415 | 126236849 | 126238417 | 156831 |
| 51416 | 126244784 | 126245563 | 156831 |
| 51417 | 126283048 | 126284258 | 242170, 156831 |
| 51418 | 126285059 | 126286917 | 156831 |
| 51419 | 126290205 | 126291132 | 156831 |
| 51420 | 126314283 | 126315233 | 156831 |
| 51421 | 126321485 | 126324200 | 156831 |
| 51422 | 126347106 | 126349068 | 156831 |
| 51423 | 126353780 | 126355761 | 156831 |
| 51424 | 126374316 | 126376009 | 156831 |
| 51425 | 126383421 | 126384592 | |
| 51426 | 126398306 | 126399143 | |
| 51427 | 126422319 | 126423530 | |
| 51428 | 126424167 | 126425134 | |
| 51429 | 126436277 | 126437005 | |
| 51430 | 126437969 | 126438490 | |
| 51431 | 126441038 | 126448270 | 173334 |
| 51432 | 126463653 | 126464947 | |
| 51433 | 126474873 | 126476832 | |
| 51434 | 126497709 | 126498434 | 253111 |
| 51435 | 126503400 | 126504330 | 253111 |
| 51436 | 126506419 | 126508071 | 253111 |
| 51437 | 126509442 | 126511924 | 253111 |
| 51438 | 126513371 | 126515045 | 253111 |
| 51439 | 126523548 | 126527428 | 253111 |
| 51440 | 126536840 | 126542286 | 253111 |
| 51441 | 126556119 | 126560004 | |
| 51442 | 126574636 | 126576310 | |
| 51443 | 126587168 | 126589825 | |
| 51444 | 126590192 | 126592825 | |
| 51445 | 126605873 | 126606855 | |
| 51446 | 126649039 | 126651954 | |
| 51447 | 126653492 | 126654591 | |
| 51448 | 126654830 | 126655704 | |
| 51449 | 126656003 | 126657777 | |
| 51450 | 126675106 | 126677762 | |
| 51451 | 126697812 | 126700233 | |
| 51452 | 126708891 | 126711345 | |
| 51453 | 126798052 | 126799355 | |
| 51454 | 127466024 | 127466845 | |
| 51455 | 127543473 | 127544944 | |
| 51456 | 127568429 | 127571712 | 254286, 254010, 168672 |
| 51457 | 127583616 | 127585002 | 254286 |
| 51458 | 127766608 | 127767523 | |
| 51459 | 128047042 | 128048831 | |
| 51460 | 128064460 | 128065351 | |
| 51461 | 128088847 | 128089969 | 253264 |
| 51462 | 128217639 | 128220074 | |
| 51463 | 128221405 | 128233441 | 247844 |
| 51464 | 128233994 | 128238580 | |
| 51465 | 128319579 | 128320272 | 246228 |
| 51466 | 128745975 | 128752288 | 136997, 249375 |
| 51467 | 128752562 | 128757128 | 136997 |
| 51468 | 128805876 | 128808803 | 249859 |
| 51469 | 128845132 | 128845724 | 249859 |
| 51470 | 128863081 | 128868060 | 249859 |
| 51471 | 128901965 | 128904955 | 249859 |
| 51472 | 128905896 | 128907749 | 249859 |
| 51473 | 128910419 | 128912298 | 249859 |
| 51474 | 128930667 | 128931806 | 249859 |
| 51475 | 128961690 | 128963335 | 249859 |
| 51476 | 128979405 | 128984128 | 249859 |
| 51477 | 129043359 | 129043748 | 249859 |
| 51478 | 129046466 | 129048407 | 249859 |
| 51479 | 129082151 | 129083671 | 249859 |
| 51480 | 129099483 | 129100115 | 249859 |
| 51481 | 129103031 | 129103869 | 249859 |
| 51482 | 129120865 | 129121662 | |
| 51483 | 129129590 | 129130248 | |
| 51484 | 129138746 | 129141096 | |
| 51485 | 129153606 | 129155345 | |
| 51486 | 129163754 | 129166741 | |
| 51487 | 129168268 | 129171705 | |
| 51488 | 129178300 | 129183762 | |
| 51489 | 129184044 | 129193096 | |
| 51490 | 129193534 | 129198852 | |
| 51491 | 129204963 | 129206704 | |
| 51492 | 129207359 | 129211548 | |
| 51493 | 129270335 | 129271751 | |
| 51494 | 129338533 | 129340005 | |
| 51495 | 129439660 | 129441653 | 254275 |
| 51496 | 129441862 | 129442613 | 254275 |
| 51497 | 129511708 | 129512907 | 254275 |
| 51498 | 129551255 | 129554024 | 254275 |
| 51499 | 129565584 | 129570030 | 254275 |
| 51500 | 129594687 | 129598094 | |
| 51501 | 129602965 | 129604360 | |
| 51502 | 129638247 | 129639168 | |
| 51503 | 129664885 | 129666085 | |
| 51504 | 129746342 | 129747103 | |
| 51505 | 129806212 | 129807168 | |
| 51506 | 130562134 | 130563054 | 229140 |
| 51507 | 130694086 | 130694771 | 254223 |
| 51508 | 130696739 | 130698888 | |
| 51509 | 130722729 | 130723308 | |
| 51510 | 130764930 | 130766416 | 147697 |
| 51511 | 130890663 | 130891203 | 153310 |
| 51512 | 130950991 | 130953168 | 253720, 153310 |
| 51513 | 130993731 | 130995074 | 153310 |
| 51514 | 130996496 | 130997006 | 153310 |
| 51515 | 130997549 | 130998208 | 153310 |
| 51516 | 131008439 | 131009506 | 153310 |
| 51517 | 131027012 | 131029391 | 153310 |
| 51518 | 131074095 | 131075110 | 153317 |
| 51519 | 131226331 | 131226797 | 153317 |
| 51520 | 131305342 | 131306305 | 153317 |
| 51521 | 131369532 | 131371210 | 153317 |
| 51522 | 131440167 | 131440573 | 153317 |
| 51523 | 131453978 | 131456984 | 153317 |
| 51524 | 131495351 | 131495954 | |
| 51525 | 131526454 | 131527078 | |
| 51526 | 131687616 | 131688266 | |
| 51527 | 131772073 | 131776735 | |
| 51528 | 131790569 | 131791209 | |
| 51529 | 132531152 | 132533140 | |
| 51530 | 132594302 | 132595622 | |
| 51531 | 132723783 | 132724638 | |
| 51532 | 132828015 | 132829043 | |
| 51533 | 132852662 | 132858567 | |
| 51534 | 132868252 | 132872187 | |
| 51535 | 132915324 | 132919320 | 132294 |
| 51536 | 132928071 | 132929320 | 132294 |
| 51537 | 133687159 | 133688317 | 129295 |
| 51538 | 133786809 | 133788596 | 129292 |
| 51539 | 133857742 | 133858434 | 129292 |
| 51540 | 134142921 | 134143762 | 042832 |
| 51541 | 134194589 | 134195390 | |
| 51542 | 134228570 | 134229382 | 104415 |
| 51543 | 134230021 | 134230610 | 104415 |
| 51544 | 134259405 | 134260670 | 104419 |
| 51545 | 134261607 | 134262483 | 104419 |
| 51546 | 134305487 | 134311199 | 104419 |
| 51547 | 134331821 | 134334576 | |
| 51548 | 134340701 | 134342166 | |
| 51549 | 134352897 | 134354387 | |
| 51550 | 134356168 | 134356861 | |
| 51551 | 134362419 | 134363269 | |
| 51552 | 134371891 | 134373143 | |
| 51553 | 134380512 | 134381967 | |
| 51554 | 134386879 | 134389638 | |
| 51555 | 134418791 | 134419567 | |
| 51556 | 134440630 | 134441866 | |
| 51557 | 134460593 | 134462262 | |
| 51558 | 134467767 | 134468387 | 008513 |
| 51559 | 134523488 | 134524043 | 008513 |
| 51560 | 134528949 | 134529678 | 008513 |
| 51561 | 134552159 | 134552993 | 008513 |
| 51562 | 134554500 | 134555221 | 008513 |
| 51563 | 134557917 | 134558783 | 008513 |
| 51564 | 134574611 | 134575593 | 008513 |
| 51565 | 134580197 | 134588110 | 261220, 008513 |
| 51566 | 134591575 | 134594061 | |
| 51567 | 134595138 | 134596694 | |
| 51568 | 135033348 | 135033985 | |
| 51569 | 135528212 | 135528779 | 066827 |
| 51570 | 135540188 | 135540736 | 066827 |
| 51571 | 135626287 | 135626852 | 066827 |
| 51572 | 135642723 | 135644399 | 066827 |
| 51573 | 135652989 | 135653496 | 066827 |
| 51574 | 135697427 | 135698479 | 066827 |
| 51575 | 135724100 | 135726306 | 066827 |
| 51576 | 135732121 | 135733247 | |
| 51577 | 135752622 | 135753429 | |
| 51578 | 135766030 | 135766816 | |
| 51579 | 135777497 | 135778740 | |
| 51580 | 135789814 | 135793645 | |
| 51581 | 135806231 | 135808101 | 259820 |
| 51582 | 135814026 | 135815281 | |
| 51583 | 135842779 | 135845898 | 254028 |
| 51584 | 135852759 | 135853835 | 253433 |
| 51585 | 136468060 | 136471443 | 254372, 131773 |
| 51586 | 136477945 | 136479197 | 131773 |
| 51587 | 136499738 | 136500329 | 131773 |
| 51588 | 136527585 | 136528994 | 131773 |
| 51589 | 136550392 | 136552410 | 131773 |
| 51590 | 136579551 | 136580367 | 131773 |
| 51591 | 136591722 | 136594481 | 131773 |
| 51592 | 136595375 | 136597133 | 131773 |
| 51593 | 136613962 | 136614733 | 131773 |
| 51594 | 137801541 | 137802501 | 254101 |
| 51595 | 137806517 | 137807475 | 254101 |
| 51596 | 137980276 | 137980811 | 254101 |
| 51597 | 138546328 | 138547573 | |
| 51598 | 138590879 | 138591426 | |
| 51599 | 139279664 | 139280637 | 147724 |
| 51600 | 139360898 | 139361719 | 147724 |
| 51601 | 139613797 | 139614991 | 169436 |
| 51602 | 140208701 | 140209485 | |
| 51603 | 140718002 | 140718544 | |
| 51604 | 140867045 | 140868018 | 167632 |
| 51605 | 140980434 | 140981815 | 167632 |
| 51606 | 141163690 | 141164840 | 167632 |
| 51607 | 141466930 | 141469025 | 167632 |
| 51608 | 141474133 | 141475721 | |
| 51609 | 141520279 | 141522969 | 104472 |
| 51610 | 141538335 | 141539252 | 259758 |
| 51611 | 141643959 | 141646614 | 123908 |
| 51612 | 141669335 | 141670073 | 169398 |
| 51613 | 141783731 | 141784642 | 169398 |
| 51614 | 141800262 | 141801842 | 169398 |
| 51615 | 141890033 | 141890738 | 169398 |
| 51616 | 141983060 | 141983751 | 169398 |
| 51617 | 142010103 | 142013174 | 169398 |
| 51618 | 142163737 | 142164672 | 105339 |
| 51619 | 142205963 | 142206457 | |
| 51620 | 142238392 | 142239119 | 022567 |
| 51621 | 142300295 | 142300929 | 254197, 022567 |
| 51622 | 142635166 | 142635564 | |
| 51623 | 142735906 | 142736477 | |
| 51624 | 142868667 | 142869404 | |
| 51625 | 142869866 | 142870604 | |
| 51626 | 143110293 | 143111834 | |
| 51627 | 143190294 | 143191268 | |
| 51628 | 143395210 | 143396092 | 171045 |
| 51629 | 143480515 | 143481682 | 171045 |
| 51630 | 143483433 | 143485493 | 261693, 171045 |
| 51631 | 143750139 | 143752223 | 167653, 234616 |
| 51632 | 143807201 | 143809351 | 130193, 253806, 253741 |
| 51633 | 143819980 | 143821751 | |
| 51634 | 143844099 | 143844839 | 253196 |
| 51635 | 143858188 | 143859716 | 180155 |
| 51636 | 144033482 | 144034435 | |
| 51637 | 144094374 | 144096007 | 247317 |
| 51638 | 144097314 | 144097860 | 247317 |
| 51639 | 144099593 | 144100639 | 247317, 160932 |
| 51640 | 144328255 | 144330285 | 264668, 181638 |
| 51641 | 144348559 | 144350561 | 250571, 264668 |
| 51642 | 144358478 | 144359480 | 264668, 250571 |
| 51643 | 144362358 | 144364172 | 253716 |
| 51644 | 144372365 | 144373932 | 272172, 185730 |
| 51645 | 144416247 | 144418239 | 184428 |
| 51646 | 144450075 | 144451162 | 158106, 254389 |
| 51647 | 144471156 | 144473052 | |
| 51648 | 144536644 | 144537180 | 014164 |
| 51649 | 144609422 | 144610786 | 014164 |
| 51650 | 144622880 | 144624560 | 254859, 254144, 014164 |
| 51651 | 144629292 | 144629901 | 254859 |
| 51652 | 144634121 | 144636483 | 104518 |
| 51653 | 144639941 | 144641571 | 104518 |
| 51654 | 144646914 | 144647708 | |
| 51655 | 144659278 | 144661548 | 255050, 147813 |
| 51656 | 144678713 | 144680875 | 179886, 104529 |
| 51657 | 144690422 | 144692245 | 104524 |
| 51658 | 144698795 | 144700884 | 104522 |
| 51659 | 144717361 | 144719151 | 183309 |
| 51660 | 144766241 | 144767474 | 181135 |
| 51661 | 144814425 | 144816705 | 265660, 203499, 180921 |
| 51662 | 144896712 | 144898728 | 179950, 180900 |
| 51663 | 144906012 | 144907221 | 179950 |
| 51664 | 144910064 | 144913412 | 179950 |
| 51665 | 144914686 | 144919186 | 255343, 185189 |
| 51666 | 144921857 | 144923497 | 185189 |
| 51667 | 144952534 | 144953068 | 227184 |
| 51668 | 144973272 | 144974961 | |
| 51669 | 145010450 | 145012539 | 178209 |
| 51670 | 145016134 | 145018592 | 178209 |
| 51671 | 145026094 | 145027452 | 178209 |
| 51672 | 145030425 | 145031572 | 178209 |
| 51673 | 145043118 | 145048020 | 178209 |
| 51674 | 145048832 | 145049620 | 178209 |
| 51675 | 145050189 | 145051715 | 178685, 178209 |
| 51676 | 145059577 | 145061480 | 178685 |
| 51677 | 145063992 | 145066088 | 178719, 178685 |
| 51678 | 145076847 | 145077963 | 178685 |
| 51679 | 145114670 | 145116390 | 178814 |
| 51680 | 145117658 | 145119021 | 178814 |
| 51681 | 145132551 | 145134588 | 178896, 255224 |
| 51682 | 145136690 | 145138422 | 197858 |
| 51683 | 145149632 | 145151406 | 179091 |
| 51684 | 145157672 | 145160462 | 179632, 179526 |
| 51685 | 145514350 | 145516409 | 185122, 170727 |
| 51686 | 145549411 | 145551162 | 185000 |
| 51687 | 145581133 | 145583283 | 182325, 185803 |
| 51688 | 145596674 | 145598721 | 173137 |
| 51689 | 145633792 | 145635635 | 272115, 147804, 071894 |
| 51690 | 145652641 | 145655984 | 160949, 160948 |
| 51691 | 145669565 | 145670705 | 160949 |
| 51692 | 145680849 | 145683821 | 187954 |
| 51693 | 145686820 | 145689081 | 187954 |
| 51694 | 145690031 | 145691215 | 187954, 254578 |
| 51695 | 145691573 | 145692690 | 167702 |
| 51696 | 145702722 | 145704614 | 160972 |
| 51697 | 145726078 | 145730851 | 167701, 160972, 255182 |
| 51698 | 145732710 | 145735524 | 167700 |
| 51699 | 145743068 | 145744162 | 160959, 160957 |
| 51700 | 145747179 | 145748957 | 254402, 160959 |
| 51701 | 145753756 | 145755164 | 147799, 213563 |
| 51702 | 145910690 | 145912152 | 147799 |
| 51703 | 145979914 | 145981777 | 198169 |
| 51704 | 146011562 | 146013553 | 196378 |
| 51705 | 146016614 | 146018891 | 161016 |
| 51706 | 146023639 | 146025294 | 197363 |
| 51707 | 146051954 | 146053832 | 147789 |
| 51708 | 146077356 | 146079302 | 263640, 170619, 196150 |
| 51709 | 146125424 | 146128778 | 196150 |
| 51710 | 146175485 | 146176666 | 170631 |
| 51711 | 146227660 | 146229315 | 255559, 196922 |
| 51712 | 146277168 | 146277535 | |
| 51713 | 146277601 | 146278878 | 182307 |

The locations of ODSCs on Chromosome 9 are shown in Table 51.

TABLE 51
ODSC Locations on Chromosome 9

| SEQ ID NO. | Start | End | Overlapping Gene(s) |
|---|---|---|---|
| 51714 | 463634 | 464365 | 107099, 227155 |
| 51715 | 489027 | 490094 | 107104, 228115, 227155 |
| 51716 | 682175 | 688135 | 227914, 107104 |
| 51717 | 692770 | 698825 | 107104 |
| 51718 | 703884 | 704660 | 107104 |
| 51719 | 705442 | 707829 | 107104 |
| 51720 | 708017 | 710686 | 107104 |
| 51721 | 737681 | 739554 | 107104 |
| 51722 | 742700 | 743456 | 107104 |
| 51723 | 744399 | 745577 | 107104 |
| 51724 | 745679 | 746331 | 107104 |
| 51725 | 755639 | 756483 | |
| 51726 | 761439 | 761902 | |
| 51727 | 1172904 | 1173799 | |
| 51728 | 1200685 | 1201924 | |
| 51729 | 1232189 | 1233005 | |
| 51730 | 1978884 | 1980868 | |
| 51731 | 2013845 | 2019070 | 080503 |
| 51732 | 2025213 | 2025997 | 080503 |
| 51733 | 2414436 | 2414956 | |
| 51734 | 2427059 | 2428148 | 236404 |
| 51735 | 2465701 | 2467367 | 236404 |
| 51736 | 2559223 | 2559935 | 236404 |
| 51737 | 2568151 | 2569162 | 236404 |
| 51738 | 2570286 | 2571452 | 236404 |
| 51739 | 2602721 | 2603105 | 236404 |
| 51740 | 2621292 | 2624520 | 147852, 236404 |
| 51741 | 2720150 | 2720819 | 080608, 168263 |
| 51742 | 2727643 | 2728678 | 168263, 080608 |
| 51743 | 2802450 | 2802967 | 080608 |
| 51744 | 2808901 | 2811141 | 080608 |
| 51745 | 2842803 | 2845282 | 080608 |
| 51746 | 3359275 | 3360762 | 080298 |
| 51747 | 3508284 | 3509466 | 080298 |
| 51748 | 3525093 | 3528105 | 232104, 080298 |
| 51749 | 3959934 | 3963851 | 107249 |
| 51750 | 3992814 | 3993299 | 107249 |
| 51751 | 4065997 | 4067063 | 107249 |
| 51752 | 4068067 | 4069028 | 107249 |
| 51753 | 4126200 | 4127207 | 107249 |
| 51754 | 4127894 | 4128373 | 107249 |
| 51755 | 4136124 | 4139235 | 107249 |
| 51756 | 4141220 | 4141789 | 107249 |
| 51757 | 4143498 | 4148639 | 107249 |
| 51758 | 4154395 | 4156907 | 107249 |
| 51759 | 4174566 | 4175796 | 107249 |
| 51760 | 4235597 | 4237033 | 107249 |
| 51761 | 4257940 | 4259977 | 107249 |
| 51762 | 4261548 | 4263839 | 107249 |
| 51763 | 4292245 | 4294524 | 107249 |
| 51764 | 4296158 | 4300509 | 228322, 107249 |
| 51765 | 4301092 | 4303368 | 107249, 228322 |
| 51766 | 4304559 | 4307578 | 228322, 107249 |
| 51767 | 4318244 | 4319492 | 107249 |
| 51768 | 4334250 | 4335064 | 107249 |
| 51769 | 4339087 | 4339771 | 107249 |
| 51770 | 4339894 | 4340687 | 107249 |
| 51771 | 4417496 | 4418592 | |
| 51772 | 4420214 | 4420879 | |
| 51773 | 4430444 | 4431380 | |
| 51774 | 4489188 | 4492512 | 106688 |
| 51775 | 4503955 | 4504715 | 106688 |
| 51776 | 4507140 | 4511556 | 106688 |
| 51777 | 4549126 | 4551604 | 106688 |
| 51778 | 4553876 | 4554545 | 106688, 106686 |
| 51779 | 4578964 | 4579553 | 106688, 106686 |
| 51780 | 4584297 | 4585126 | 106688, 106686 |
| 51781 | 4596061 | 4597009 | 106686 |
| 51782 | 4657734 | 4658268 | 106686 |
| 51783 | 4661187 | 4664329 | 205808, 106686 |
| 51784 | 4665301 | 4667214 | 106686 |
| 51785 | 4677199 | 4681870 | 106993, 273061 |
| 51786 | 4699853 | 4700468 | 106993 |
| 51787 | 4738823 | 4742566 | 147853 |
| 51788 | 4742949 | 4744007 | |
| 51789 | 4792276 | 4794334 | 120158 |
| 51790 | 4828552 | 4835870 | 120158 |
| 51791 | 4835914 | 4843690 | 120158 |
| 51792 | 4861788 | 4862552 | 120158 |
| 51793 | 4983824 | 4986708 | 096968 |
| 51794 | 5436013 | 5438536 | 107020 |
| 51795 | 5449546 | 5451348 | 120217 |
| 51796 | 5608663 | 5609396 | |
| 51797 | 5628152 | 5630583 | 107036 |
| 51798 | 5633204 | 5633581 | 107036 |
| 51799 | 5783958 | 5784408 | 099219 |
| 51800 | 5815359 | 5815856 | 099219 |
| 51801 | 5817826 | 5818397 | 099219 |
| 51802 | 5832011 | 5833312 | 099219 |
| 51803 | 5838594 | 5840214 | |
| 51804 | 5879506 | 5879989 | |
| 51805 | 5893756 | 5894382 | 183354, 120215 |
| 51806 | 6007333 | 6008877 | 263575, 183354 |
| 51807 | 6014729 | 6016051 | 137040 |
| 51808 | 6080089 | 6080753 | |
| 51809 | 6141930 | 6144043 | |
| 51810 | 6271353 | 6272811 | |
| 51811 | 6412579 | 6414747 | 147854 |
| 51812 | 6590696 | 6591251 | 178445 |
| 51813 | 6628731 | 6629217 | 178445 |
| 51814 | 6640018 | 6642363 | 178445 |
| 51815 | 6645578 | 6646929 | 236924, 178445 |
| 51816 | 6663919 | 6665454 | 236924 |
| 51817 | 6676154 | 6676944 | |
| 51818 | 6680135 | 6682478 | |
| 51819 | 6703257 | 6704561 | 229611 |
| 51820 | 6715115 | 6717176 | 225489 |
| 51821 | 6756766 | 6759819 | 107077 |
| 51822 | 6787345 | 6789272 | 107077 |
| 51823 | 6902341 | 6902932 | 224972, 107077 |
| 51824 | 6931151 | 6933045 | 107077, 224972 |
| 51825 | 6943371 | 6945354 | 107077, 224972 |
| 51826 | 7153598 | 7153966 | 107077 |
| 51827 | 7185457 | 7186040 | |
| 51828 | 7799110 | 7800158 | 137038 |
| 51829 | 8600867 | 8601754 | 153707 |
| 51830 | 8660165 | 8660827 | 153707 |
| 51831 | 8821487 | 8823986 | 153707 |
| 51832 | 8856862 | 8858343 | 225706, 153707 |
| 51833 | 8870509 | 8871418 | 153707 |
| 51834 | 8958019 | 8959162 | 153707, 234021 |
| 51835 | 8959822 | 8963232 | 234021, 153707 |
| 51836 | 9054505 | 9055077 | 153707 |
| 51837 | 9752531 | 9753221 | 153707 |
| 51838 | 10146239 | 10147260 | 153707 |
| 51839 | 10250802 | 10252678 | 153707 |
| 51840 | 12759683 | 12760207 | 235448 |
| 51841 | 12774511 | 12779191 | 153714, 235448 |
| 51842 | 12783092 | 12786511 | 153714, 235448 |
| 51843 | 12793888 | 12794867 | 153714, 235448 |
| 51844 | 12796748 | 12799218 | 153714, 235448 |
| 51845 | 12811377 | 12820051 | 235448, 153714 |
| 51846 | 13033120 | 13035318 | |
| 51847 | 13102635 | 13103177 | |
| 51848 | 13140755 | 13141966 | 107186 |
| 51849 | 13149412 | 13149936 | 107186 |
| 51850 | 13174534 | 13176512 | 107186 |
| 51851 | 13189475 | 13190656 | 107186 |
| 51852 | 13203105 | 13203913 | 107186 |
| 51853 | 13278547 | 13280119 | 234740, 107186 |
| 51854 | 14178647 | 14181641 | 147862 |
| 51855 | 14198795 | 14199636 | 147862 |
| 51856 | 14202907 | 14204282 | 234982, 147862 |
| 51857 | 14204821 | 14206780 | 234982, 147862 |
| 51858 | 14211349 | 14214422 | 147862 |
| 51859 | 14220976 | 14222683 | 147862 |
| 51860 | 14238018 | 14238988 | 147862 |
| 51861 | 14250793 | 14252131 | 147862 |
| 51862 | 14291975 | 14292773 | 147862 |
| 51863 | 14294473 | 14295612 | 147862 |
| 51864 | 14301627 | 14303215 | 147862 |
| 51865 | 14311895 | 14316800 | 147862 |
| 51866 | 14319977 | 14321486 | 225472, 147862 |
| 51867 | 14321532 | 14323434 | 225472, 147862 |
| 51868 | 14372705 | 14373285 | 147862 |
| 51869 | 14692553 | 14694055 | 175893 |
| 51870 | 15295427 | 15297257 | 155158 |
| 51871 | 15305969 | 15307699 | 155158 |
| 51872 | 15329665 | 15330936 | |
| 51873 | 15333134 | 15333953 | |
| 51874 | 15421990 | 15424280 | 164975 |
| 51875 | 15510108 | 15511568 | 164985 |
| 51876 | 15551983 | 15553261 | 164989 |
| 51877 | 15974866 | 15976238 | 164989 |
| 51878 | 16133573 | 16134409 | |
| 51879 | 16213600 | 16214497 | 205549 |
| 51880 | 16241215 | 16241758 | 205549 |
| 51881 | 16245637 | 16248527 | 205549 |
| 51882 | 16266619 | 16268447 | 205549 |
| 51883 | 16275535 | 16276917 | 205549 |
| 51884 | 16335355 | 16338736 | |
| 51885 | 16385558 | 16386848 | |
| 51886 | 16412283 | 16415569 | 173068 |
| 51887 | 16444869 | 16446689 | 173068 |
| 51888 | 16653476 | 16654333 | 173068 |
| 51889 | 17134788 | 17136128 | 044459 |
| 51890 | 17138357 | 17139086 | 044459 |
| 51891 | 17230349 | 17231251 | 044459 |
| 51892 | 17366890 | 17367466 | 044459 |
| 51893 | 17422933 | 17424041 | 044459 |
| 51894 | 17436222 | 17437404 | 044459 |
| 51895 | 18002358 | 18003732 | |
| 51896 | 18207984 | 18208643 | |
| 51897 | 18452005 | 18453000 | |
| 51898 | 18456269 | 18457255 | |
| 51899 | 18605643 | 18606301 | 178031 |
| 51900 | 19048408 | 19050449 | 155876 |
| 51901 | 19102088 | 19103734 | 147874 |
| 51902 | 19113564 | 19115354 | 147872 |
| 51903 | 19118559 | 19119789 | 147872 |
| 51904 | 19125756 | 19128467 | 147872 |
| 51905 | 19141930 | 19143496 | 147872 |
| 51906 | 19151274 | 19152642 | |
| 51907 | 19153213 | 19156953 | |
| 51908 | 19160827 | 19163309 | |
| 51909 | 19173382 | 19174738 | |
| 51910 | 19229675 | 19231956 | 137145 |
| 51911 | 19378975 | 19381227 | 137154 |
| 51912 | 19925831 | 19926621 | 261402 |
| 51913 | 20550698 | 20551805 | 171843 |
| 51914 | 20611669 | 20612517 | 171843 |
| 51915 | 20621615 | 20622693 | 171843 |
| 51916 | 20624523 | 20625237 | |
| 51917 | 20683886 | 20685754 | 227071, 188352 |
| 51918 | 20835323 | 20836282 | 188352 |
| 51919 | 21292833 | 21295824 | |
| 51920 | 21334295 | 21335985 | 198642 |
| 51921 | 21591564 | 21592714 | |
| 51922 | 21801564 | 21803931 | 264545, 099810 |
| 51923 | 21812416 | 21814588 | 264545, 099810 |
| 51924 | 21831548 | 21834057 | 264545, 099810 |
| 51925 | 21835605 | 21837441 | 264545, 099810 |
| 51926 | 21852660 | 21855896 | 264545, 099810 |
| 51927 | 21856172 | 21857261 | 264545, 099810 |
| 51928 | 21872144 | 21874376 | 264545, 099810 |
| 51929 | 21964953 | 21965951 | 264545 |
| 51930 | 21994038 | 21996126 | 266446, 240498, 147889, |
| | | | 264545 |
| 51931 | 22007983 | 22009913 | 147883, 264545, 240498 |
| 51932 | 22030215 | 22030694 | 264545, 240498 |
| 51933 | 22079420 | 22080877 | 240498 |
| 51934 | 22084095 | 22085262 | 240498 |
| 51935 | 22088302 | 22089279 | 240498 |
| 51936 | 22098382 | 22099594 | 240498 |
| 51937 | 22101885 | 22104819 | 240498 |
| 51938 | 22106495 | 22109534 | 240498 |
| 51939 | 22158883 | 22159415 | |
| 51940 | 22237597 | 22239397 | |
| 51941 | 22342539 | 22343150 | |
| 51942 | 22446461 | 22449448 | 176399 |
| 51943 | 22453574 | 22454388 | 176399 |
| 51944 | 22462789 | 22463865 | |
| 51945 | 22530936 | 22531503 | |
| 51946 | 22683072 | 22684666 | 234840 |
| 51947 | 23058232 | 23058657 | |
| 51948 | 23172943 | 23173652 | |
| 51949 | 23309001 | 23310152 | |
| 51950 | 23318958 | 23319589 | |
| 51951 | 23326040 | 23327773 | |
| 51952 | 23341315 | 23344118 | |
| 51953 | 23431331 | 23432253 | |
| 51954 | 23438345 | 23439072 | |
| 51955 | 23537149 | 23537739 | |
| 51956 | 25207289 | 25207887 | |
| 51957 | 25676847 | 25679073 | 198680 |
| 51958 | 26869766 | 26870783 | 120159 |
| 51959 | 26891272 | 26893662 | 120159 |
| 51960 | 26946084 | 26948540 | 096872, 137055 |
| 51961 | 26955065 | 26957166 | 234676, 096872 |
| 51962 | 27379536 | 27380843 | 120162 |
| 51963 | 27436064 | 27436947 | 120162 |
| 51964 | 27462268 | 27462980 | 120162 |
| 51965 | 27527979 | 27530071 | 120162 |
| 51966 | 27536889 | 27537560 | |
| 51967 | 27538754 | 27541433 | |
| 51968 | 27565885 | 27566628 | 147894 |
| 51969 | 27571923 | 27574624 | 147894 |
| 51970 | 27851667 | 27852313 | |
| 51971 | 27864642 | 27865938 | |
| 51972 | 27869507 | 27871257 | |
| 51973 | 28779194 | 28779746 | |
| 51974 | 30628868 | 30629627 | |
| 51975 | 31136295 | 31138683 | |
| 51976 | 31303597 | 31305040 | |
| 51977 | 31824811 | 31825429 | |
| 51978 | 32304357 | 32305359 | |
| 51979 | 32383765 | 32385523 | 122729 |
| 51980 | 32393806 | 32397075 | 122729 |
| 51981 | 32401458 | 32401876 | 122729 |
| 51982 | 32425385 | 32427937 | 122729 |
| 51983 | 32486673 | 32487898 | 107201 |
| 51984 | 32525141 | 32526905 | 107201 |
| 51985 | 32550638 | 32552912 | 235453, 197579 |
| 51986 | 32572166 | 32573546 | 165264 |
| 51987 | 32741740 | 32742320 | |
| 51988 | 32954462 | 32955451 | |
| 51989 | 33000764 | 33001987 | 137074 |
| 51990 | 33024044 | 33026295 | 086061, 137074 |
| 51991 | 33029103 | 33030156 | 086061 |
| 51992 | 33043669 | 33045227 | 122692 |
| 51993 | 33075459 | 33077395 | 122692 |
| 51994 | 33081116 | 33081833 | |
| 51995 | 33101091 | 33102742 | |
| 51996 | 33106720 | 33107678 | 086062 |
| 51997 | 33139399 | 33140594 | 086062 |
| 51998 | 33156691 | 33157346 | 086062 |
| 51999 | 33158220 | 33168489 | 233554, 086062 |
| 52000 | 33170354 | 33173767 | 233554 |
| 52001 | 33185600 | 33187503 | |
| 52002 | 33263407 | 33265844 | 086065, 107262 |
| 52003 | 33289180 | 33291832 | 086102 |
| 52004 | 33293535 | 33294814 | 086102 |
| 52005 | 33300312 | 33301531 | 086102 |
| 52006 | 33304932 | 33306721 | 086102 |
| 52007 | 33320263 | 33321375 | 086102 |
| 52008 | 33327985 | 33331050 | 086102 |
| 52009 | 33391193 | 33391988 | 165269 |
| 52010 | 33415417 | 33416100 | |
| 52011 | 33424980 | 33425605 | |
| 52012 | 33442778 | 33443948 | 165272 |
| 52013 | 33445252 | 33448579 | 165272 |
| 52014 | 33458484 | 33459073 | |
| 52015 | 33473064 | 33474326 | 165271 |
| 52016 | 33510585 | 33511466 | 226823 |
| 52017 | 33813564 | 33819508 | 107341, 235481 |
| 52018 | 33931961 | 33932475 | 137073 |
| 52019 | 34035682 | 34036134 | 137073 |
| 52020 | 34047677 | 34050008 | 222259, 137073 |
| 52021 | 34072309 | 34073604 | |
| 52022 | 34125415 | 34127633 | 198876 |
| 52023 | 34178110 | 34181097 | 165006 |
| 52024 | 34185053 | 34187157 | 165006 |
| 52025 | 34226219 | 34227076 | 165006 |
| 52026 | 34329171 | 34330360 | 164978, 186638 |
| 52027 | 34343060 | 34344069 | 164978 |
| 52028 | 34352149 | 34352749 | |
| 52029 | 34374831 | 34377661 | 164976 |
| 52030 | 34380365 | 34381153 | 164972 |
| 52031 | 34403438 | 34404261 | 164970 |
| 52032 | 34457447 | 34458731 | 164970, 122735 |
| 52033 | 34543824 | 34544706 | |
| 52034 | 34606498 | 34607533 | |
| 52035 | 34611063 | 34613299 | 164967 |
| 52036 | 34630058 | 34631331 | |
| 52037 | 34636574 | 34638784 | 213930, 147955 |
| 52038 | 34641244 | 34641883 | 213930 |
| 52039 | 34644391 | 34645351 | 213930 |
| 52040 | 34646061 | 34648353 | 258728, 213930 |
| 52041 | 34650886 | 34652559 | 213930, 258728, 137070 |
| 52042 | 34659366 | 34660081 | 137070 |
| 52043 | 34664205 | 34666700 | 230074, 261215, 187186 |
| 52044 | 34688475 | 34689080 | |
| 52045 | 34701125 | 34702659 | |
| 52046 | 34717843 | 34718768 | |
| 52047 | 34961711 | 34962232 | 122733 |
| 52048 | 34988292 | 34990988 | 137094 |
| 52049 | 34991591 | 34996157 | 137094 |
| 52050 | 34996382 | 34997952 | 137094 |
| 52051 | 34998017 | 34998447 | 137094 |
| 52052 | 35025971 | 35027925 | |
| 52053 | 35041670 | 35043500 | 269510, 174038 |
| 52054 | 35070732 | 35073273 | 165280 |
| 52055 | 35079776 | 35080684 | 221829 |
| 52056 | 35095585 | 35097011 | 234181, 165282 |
| 52057 | 35101837 | 35103744 | 165283 |
| 52058 | 35110042 | 35113269 | 005238 |
| 52059 | 35114340 | 35116509 | 005238 |
| 52060 | 35161596 | 35163299 | 198722 |
| 52061 | 35229352 | 35230298 | 198722 |
| 52062 | 35257733 | 35259346 | 198722 |
| 52063 | 35470184 | 35471625 | 179766 |
| 52064 | 35488677 | 35493328 | 198853 |
| 52065 | 35509283 | 35513232 | 198853 |
| 52066 | 35515069 | 35527990 | 198853 |
| 52067 | 35529273 | 35529901 | 198853 |
| 52068 | 35603262 | 35606166 | 107140 |
| 52069 | 35646095 | 35647610 | 227933, 137101 |
| 52070 | 35656923 | 35659444 | 159884, 269900, 137135 |
| 52071 | 35668973 | 35670124 | 137135 |
| 52072 | 35688562 | 35689198 | 198467 |
| 52073 | 35690302 | 35691612 | 198467 |
| 52074 | 35727216 | 35729250 | 137076 |
| 52075 | 35730613 | 35733473 | 107175, 137076 |
| 52076 | 35746347 | 35747271 | 070610 |
| 52077 | 35747903 | 35750813 | 107185, 070610 |
| 52078 | 35756236 | 35758105 | 228843, 215183, 107185 |
| 52079 | 35790144 | 35800225 | 159899, 227388 |
| 52080 | 35801146 | 35801779 | 159899 |
| 52081 | 35811923 | 35812559 | 137098 |
| 52082 | 35813353 | 35815639 | 137103, 137133 |
| 52083 | 35828471 | 35830226 | 204930, 137103 |
| 52084 | 35833806 | 35836345 | 137103 |
| 52085 | 35905237 | 35909408 | 196196 |
| 52086 | 35910610 | 35911459 | 235387 |
| 52087 | 35923001 | 35925758 | |
| 52088 | 35929028 | 35929709 | |
| 52089 | 36008669 | 36009906 | |
| 52090 | 36036436 | 36037949 | 122707 |
| 52091 | 36090160 | 36092360 | 122707 |
| 52092 | 36092997 | 36093911 | 122707 |
| 52093 | 36101412 | 36103743 | 122707 |
| 52094 | 36166247 | 36167001 | |
| 52095 | 36189761 | 36192059 | 122705 |
| 52096 | 36257787 | 36259486 | 122705, 159921 |
| 52097 | 36319105 | 36320732 | |
| 52098 | 36331226 | 36332411 | |
| 52099 | 36399702 | 36401745 | 137075 |
| 52100 | 36401849 | 36402408 | 137075 |
| 52101 | 36419457 | 36420463 | 137075 |
| 52102 | 36486389 | 36488861 | 137075 |
| 52103 | 36493513 | 36494391 | |
| 52104 | 36572058 | 36573367 | 165304 |
| 52105 | 37079050 | 37080799 | 233137, 260100 |
| 52106 | 37119764 | 37121399 | 147905 |
| 52107 | 37414778 | 37415896 | |
| 52108 | 37416702 | 37419358 | |
| 52109 | 37421906 | 37423568 | 137106 |
| 52110 | 37429218 | 37430276 | 137106 |
| 52111 | 37430769 | 37431538 | 137106 |
| 52112 | 37464155 | 37467236 | 168795 |
| 52113 | 37471764 | 37473287 | |
| 52114 | 37485014 | 37487612 | 137054 |
| 52115 | 37507912 | 37509664 | 234160 |
| 52116 | 37575564 | 37576979 | 147912, 256966 |
| 52117 | 37591403 | 37592946 | 175768, 256966, 255872 |
| 52118 | 37602672 | 37603378 | 255872 |
| 52119 | 37753371 | 37754806 | 165275, 255872 |
| 52120 | 37784120 | 37785717 | 255872, 107371 |
| 52121 | 37800080 | 37802112 | 122741, 107371, 255872 |
| 52122 | 37902667 | 37905161 | 122696, 255872 |
| 52123 | 37907949 | 37909814 | 255872 |
| 52124 | 37913411 | 37914734 | 255872 |
| 52125 | 37921994 | 37923290 | 255872, 107338 |
| 52126 | 37951116 | 37955269 | 255872, 107338 |
| 52127 | 37963890 | 37964951 | 255872, 107338 |
| 52128 | 37966327 | 37970197 | 255872, 107338 |
| 52129 | 38007455 | 38010347 | 255872, 107338 |
| 52130 | 38015011 | 38019951 | 255872, 107338 |
| 52131 | 38023479 | 38024663 | 255872, 107338 |
| 52132 | 38025398 | 38027180 | 255872, 107338 |
| 52133 | 38031494 | 38033129 | 255872, 107338 |
| 52134 | 38035168 | 38037764 | 255872, 107338 |
| 52135 | 38045741 | 38049554 | 255872, 107338 |
| 52136 | 38057366 | 38060460 | 255872, 107338 |
| 52137 | 38062317 | 38063756 | 255872, 107338 |
| 52138 | 38065401 | 38070130 | 255872, 107338 |
| 52139 | 38226248 | 38228237 | |
| 52140 | 38380439 | 38383561 | |
| 52141 | 38385778 | 38387299 | |
| 52142 | 38388015 | 38389878 | |
| 52143 | 38392011 | 38393914 | 137124 |
| 52144 | 38434868 | 38435323 | |
| 52145 | 38436468 | 38438493 | |
| 52146 | 38442132 | 38444133 | |
| 52147 | 38619714 | 38621459 | 204860, 180071 |
| 52148 | 38682153 | 38683198 | |
| 52149 | 38713305 | 38714037 | |
| 52150 | 68455223 | 68455928 | 215548 |
| 52151 | 71115616 | 71115946 | 154330 |
| 52152 | 71123311 | 71123848 | 154330 |
| 52153 | 71130286 | 71130843 | 154330 |
| 52154 | 71145444 | 71146181 | 154330 |
| 52155 | 71394106 | 71396098 | 187866, 107242 |
| 52156 | 71649947 | 71652100 | 165060 |
| 52157 | 71693345 | 71694065 | 165060 |
| 52158 | 71708393 | 71709053 | 165060 |
| 52159 | 71735145 | 71738102 | 119139 |
| 52160 | 71768514 | 71769553 | 119139 |
| 52161 | 71770180 | 71770857 | 119139 |
| 52162 | 71783746 | 71784280 | 119139 |
| 52163 | 71788047 | 71796624 | 119139 |
| 52164 | 71991278 | 71991989 | 135063 |
| 52165 | 72083277 | 72083662 | 107282 |
| 52166 | 72084723 | 72086849 | 107282 |
| 52167 | 72188389 | 72189748 | 107282 |
| 52168 | 72194750 | 72196383 | 107282 |
| 52169 | 72247575 | 72248116 | 107282 |
| 52170 | 72271193 | 72273386 | 107282 |
| 52171 | 72285796 | 72288607 | 261447, 107282 |
| 52172 | 72362667 | 72364471 | 188647 |
| 52173 | 72373267 | 72375742 | 188647 |
| 52174 | 72472766 | 72473251 | 204711 |
| 52175 | 72500220 | 72501088 | 204711 |
| 52176 | 72509168 | 72510952 | 204711 |
| 52177 | 72560921 | 72562642 | |
| 52178 | 72872429 | 72875032 | 198887, 268364 |
| 52179 | 73021677 | 73022505 | 119138 |
| 52180 | 73024828 | 73030548 | 119138 |
| 52181 | 73032824 | 73037112 | |
| 52182 | 73084593 | 73086629 | |
| 52183 | 73089299 | 73097099 | |
| 52184 | 73098423 | 73101074 | |
| 52185 | 73539595 | 73540396 | 083067 |
| 52186 | 73657332 | 73657993 | 083067 |
| 52187 | 73660713 | 73661080 | 083067 |
| 52188 | 73711135 | 73712172 | 083067 |
| 52189 | 73893990 | 73894929 | 083067 |
| 52190 | 74202012 | 74204049 | |
| 52191 | 74223683 | 74224687 | |
| 52192 | 74242375 | 74242935 | |
| 52193 | 74292069 | 74297314 | |
| 52194 | 74307128 | 74307671 | 135048 |
| 52195 | 74330903 | 74331806 | 135048 |
| 52196 | 74340741 | 74341954 | 135048 |
| 52197 | 74373037 | 74374831 | 135048 |
| 52198 | 74381295 | 74385615 | 135048 |
| 52199 | 74392922 | 74393773 | 135048 |
| 52200 | 74407427 | 74409457 | 135048 |
| 52201 | 74524981 | 74527554 | 155621, 107362 |
| 52202 | 74655958 | 74659646 | |
| 52203 | 74742219 | 74749337 | 119125 |
| 52204 | 74755004 | 74756121 | 119125 |
| 52205 | 74763235 | 74766540 | 119125 |
| 52206 | 74768825 | 74771879 | 119125 |
| 52207 | 74828830 | 74829297 | 119125 |
| 52208 | 74908817 | 74910957 | |
| 52209 | 74912198 | 74913190 | |
| 52210 | 74913838 | 74914542 | |
| 52211 | 74976899 | 74981990 | 107372 |
| 52212 | 75010983 | 75012140 | |
| 52213 | 75075075 | 75076892 | |
| 52214 | 75087789 | 75089681 | |
| 52215 | 75092850 | 75093690 | |
| 52216 | 75168682 | 75170026 | 165091 |
| 52217 | 75171668 | 75173470 | 165091 |
| 52218 | 75191692 | 75193915 | 165091 |
| 52219 | 75201042 | 75202093 | 165091 |
| 52220 | 75471903 | 75474873 | |
| 52221 | 75540122 | 75541193 | 165092 |
| 52222 | 75548951 | 75550034 | 165092 |
| 52223 | 75561702 | 75568874 | 165092 |
| 52224 | 75593242 | 75594143 | 165092 |
| 52225 | 75596436 | 75600562 | 165092 |
| 52226 | 75611010 | 75613558 | 165092 |
| 52227 | 75637541 | 75638632 | 165092 |
| 52228 | 75652507 | 75654146 | 165092 |
| 52229 | 75665032 | 75667864 | 165092, 228024 |
| 52230 | 75667942 | 75669390 | 165092, 228024 |
| 52231 | 75688113 | 75690100 | 165092 |
| 52232 | 75698148 | 75698835 | |
| 52233 | 75764378 | 75764796 | |
| 52234 | 75885961 | 75886474 | |
| 52235 | 76160409 | 76160934 | |
| 52236 | 76298539 | 76300037 | |
| 52237 | 76639959 | 76641268 | |
| 52238 | 76722521 | 76723069 | |
| 52239 | 77375295 | 77375668 | 119121 |
| 52240 | 77566496 | 77568161 | 203321, 135045 |
| 52241 | 77641888 | 77643869 | 156017 |
| 52242 | 77701489 | 77704407 | 134996, 106733 |
| 52243 | 77796636 | 77797122 | |
| 52244 | 77844352 | 77845882 | |
| 52245 | 77850249 | 77851076 | |
| 52246 | 77882480 | 77883438 | |
| 52247 | 78504587 | 78507648 | 099139 |
| 52248 | 78518768 | 78519910 | 099139 |
| 52249 | 78522110 | 78523692 | 099139 |
| 52250 | 78565591 | 78566533 | 099139 |
| 52251 | 78567988 | 78568724 | 099139 |
| 52252 | 78589224 | 78589653 | 099139 |
| 52253 | 78611403 | 78612442 | 099139 |
| 52254 | 78712213 | 78713013 | 099139 |
| 52255 | 78870789 | 78871509 | 099139 |
| 52256 | 78959974 | 78960486 | 099139 |
| 52257 | 78979951 | 78980379 | |
| 52258 | 78986840 | 78990854 | |
| 52259 | 79008323 | 79010434 | 135002 |
| 52260 | 79017981 | 79019514 | |
| 52261 | 79186458 | 79186959 | 241781 |
| 52262 | 79785774 | 79786664 | |
| 52263 | 79791563 | 79794320 | 197969, 232998 |
| 52264 | 80081510 | 80082520 | 231373, 156049 |
| 52265 | 80127619 | 80128315 | 231373, 156049 |
| 52266 | 80161223 | 80161812 | 156049 |
| 52267 | 80263024 | 80265131 | 156049 |
| 52268 | 80278452 | 80279230 | |
| 52269 | 80292809 | 80293902 | |
| 52270 | 80294070 | 80295466 | |
| 52271 | 80322145 | 80324065 | |
| 52272 | 80421584 | 80424821 | 156052 |
| 52273 | 80576256 | 80576898 | 156052 |
| 52274 | 80600715 | 80601406 | 156052 |
| 52275 | 80618714 | 80620075 | 156052 |
| 52276 | 80637737 | 80638556 | 156052 |
| 52277 | 80644993 | 80648191 | 156052 |
| 52278 | 80748679 | 80749204 | |
| 52279 | 80836149 | 80836806 | |
| 52280 | 80850159 | 80851802 | 148019 |
| 52281 | 80893267 | 80894060 | 148019 |
| 52282 | 80910832 | 80913653 | 135069 |
| 52283 | 81021568 | 81022393 | |
| 52284 | 82186348 | 82186992 | 106829 |
| 52285 | 82187030 | 82189188 | 106829 |
| 52286 | 82285435 | 82286061 | 106829 |
| 52287 | 83167757 | 83168291 | |
| 52288 | 83740273 | 83741346 | |
| 52289 | 83920529 | 83921463 | |
| 52290 | 83929035 | 83930123 | |
| 52291 | 84003710 | 84005991 | |
| 52292 | 84008534 | 84010301 | |
| 52293 | 84136543 | 84137585 | |
| 52294 | 84137658 | 84138715 | |
| 52295 | 84144236 | 84145654 | |
| 52296 | 84148709 | 84152770 | |
| 52297 | 84164248 | 84165518 | |
| 52298 | 84172872 | 84175472 | |
| 52299 | 84176895 | 84179022 | |
| 52300 | 84179295 | 84180858 | |
| 52301 | 84202032 | 84204655 | 196781 |
| 52302 | 84241259 | 84241908 | 196781 |
| 52303 | 84243021 | 84244850 | 196781 |
| 52304 | 84270928 | 84272281 | 196781 |
| 52305 | 84300801 | 84303380 | 196781 |
| 52306 | 84304224 | 84305728 | 233926 |
| 52307 | 84351909 | 84354026 | 233926 |
| 52308 | 84402239 | 84403477 | |
| 52309 | 84434691 | 84435639 | |
| 52310 | 84457432 | 84458170 | |
| 52311 | 84634641 | 84637940 | |
| 52312 | 84928304 | 84929209 | 228430 |
| 52313 | 84946575 | 84948040 | 228430 |
| 52314 | 84982751 | 84985309 | 228430 |
| 52315 | 85024883 | 85026582 | 228430 |
| 52316 | 85069374 | 85070648 | 232749, 228430 |
| 52317 | 85103347 | 85104226 | 228430 |
| 52318 | 85173227 | 85174661 | 228430 |
| 52319 | 85175210 | 85175824 | 228430 |
| 52320 | 85212104 | 85213299 | |
| 52321 | 85219582 | 85220807 | |
| 52322 | 85221164 | 85222144 | |
| 52323 | 85549458 | 85551288 | |
| 52324 | 85676178 | 85678196 | 165105 |
| 52325 | 85734377 | 85735742 | |
| 52326 | 85744454 | 85749575 | |
| 52327 | 85761104 | 85761693 | |
| 52328 | 85769069 | 85770209 | |
| 52329 | 85834065 | 85835079 | 229109 |
| 52330 | 86046544 | 86047748 | 172159 |
| 52331 | 86153082 | 86154001 | 172159 |
| 52332 | 86226399 | 86227137 | |
| 52333 | 86235593 | 86236646 | |
| 52334 | 86237281 | 86239427 | 148057 |
| 52335 | 86244394 | 86245382 | 148057 |
| 52336 | 86252275 | 86252663 | 148057 |
| 52337 | 86321502 | 86324354 | 254473, 135018 |
| 52338 | 86500445 | 86500885 | 165115 |
| 52339 | 86535128 | 86536894 | 165115 |
| 52340 | 86548512 | 86550506 | |
| 52341 | 86570221 | 86572511 | 165118 |
| 52342 | 86576968 | 86577845 | |
| 52343 | 86593400 | 86596793 | 178966, 165119 |
| 52344 | 86601138 | 86602687 | 178966 |
| 52345 | 86607141 | 86607982 | 202523, 178966 |
| 52346 | 86911003 | 86911605 | 197506 |
| 52347 | 86986311 | 86987045 | |
| 52348 | 87096685 | 87097289 | |
| 52349 | 87127023 | 87128958 | |
| 52350 | 88355652 | 88357895 | 135049 |
| 52351 | 88555152 | 88557395 | 135040 |
| 52352 | 88677665 | 88678699 | 135052 |
| 52353 | 88708587 | 88709656 | 135052 |
| 52354 | 88713706 | 88715291 | 135052 |
| 52355 | 88721729 | 88722266 | |
| 52356 | 88742128 | 88742863 | |
| 52357 | 88822471 | 88822970 | |
| 52358 | 88896166 | 88898733 | 135070 |
| 52359 | 88954343 | 88957482 | 083223 |
| 52360 | 88963033 | 88965994 | 083223 |
| 52361 | 88966325 | 88970453 | 083223 |
| 52362 | 88983337 | 88985458 | |
| 52363 | 89011317 | 89012842 | |
| 52364 | 89018605 | 89023479 | |
| 52365 | 89067001 | 89067688 | |
| 52366 | 89561179 | 89562815 | 180447 |
| 52367 | 89763197 | 89763534 | |
| 52368 | 89989535 | 89990982 | |
| 52369 | 89995961 | 89996565 | |
| 52370 | 90010865 | 90011540 | |
| 52371 | 90037633 | 90039879 | 228373 |
| 52372 | 90055662 | 90056894 | |
| 52373 | 90094562 | 90095323 | |
| 52374 | 90112233 | 90115364 | 196730 |
| 52375 | 90120464 | 90121691 | 196730 |
| 52376 | 90128976 | 90129439 | 196730 |
| 52377 | 90178792 | 90180105 | 196730 |
| 52378 | 90183824 | 90185743 | 196730 |
| 52379 | 90206742 | 90208246 | 196730 |
| 52380 | 90227995 | 90229200 | 196730 |
| 52381 | 90255324 | 90255825 | 196730 |
| 52382 | 90299346 | 90300012 | 196730 |
| 52383 | 90318258 | 90319922 | 196730 |
| 52384 | 90326096 | 90329394 | |
| 52385 | 90329684 | 90330186 | |
| 52386 | 90340516 | 90342105 | 135047 |
| 52387 | 90407642 | 90408709 | |
| 52388 | 90569032 | 90569736 | |
| 52389 | 90574788 | 90576083 | |
| 52390 | 90588798 | 90590052 | 156345 |
| 52391 | 90690693 | 90692515 | |
| 52392 | 90704897 | 90705861 | |
| 52393 | 91001990 | 91004524 | 106723 |
| 52394 | 91532170 | 91532718 | |
| 52395 | 91750359 | 91751055 | 148082 |
| 52396 | 91794366 | 91795818 | |
| 52397 | 91924595 | 91926336 | 123975 |
| 52398 | 91932154 | 91934197 | 187742 |
| 52399 | 91938132 | 91938865 | 187742 |
| 52400 | 91965886 | 91966741 | 187742 |
| 52401 | 92001552 | 92002435 | 187764 |
| 52402 | 92002929 | 92003552 | 187764 |
| 52403 | 92134062 | 92134885 | |
| 52404 | 92170467 | 92172319 | |
| 52405 | 92212948 | 92214485 | |
| 52406 | 92217808 | 92222265 | 130222 |
| 52407 | 92262786 | 92264425 | |
| 52408 | 92268862 | 92269607 | |
| 52409 | 92270064 | 92271436 | |
| 52410 | 92276424 | 92277570 | |
| 52411 | 92282613 | 92283385 | |
| 52412 | 92284175 | 92284783 | |
| 52413 | 92290891 | 92291615 | |
| 52414 | 92300387 | 92302382 | |
| 52415 | 92311537 | 92312571 | |
| 52416 | 92314260 | 92315253 | |
| 52417 | 92458147 | 92459128 | |
| 52418 | 92479442 | 92480717 | |
| 52419 | 92494863 | 92495733 | |
| 52420 | 92685996 | 92686759 | |
| 52421 | 92755732 | 92758782 | |
| 52422 | 92761475 | 92763778 | |
| 52423 | 92775034 | 92775643 | |
| 52424 | 92787609 | 92788560 | 227555 |
| 52425 | 92817070 | 92817485 | |
| 52426 | 92826202 | 92828072 | |
| 52427 | 92869256 | 92869822 | |
| 52428 | 92931779 | 92932404 | |
| 52429 | 93093879 | 93095464 | 231107 |
| 52430 | 93204129 | 93204560 | |
| 52431 | 93220501 | 93221404 | |
| 52432 | 93370017 | 93370961 | |
| 52433 | 93683406 | 93683962 | |
| 52434 | 93687985 | 93688464 | |
| 52435 | 93752609 | 93755678 | |
| 52436 | 93761530 | 93764542 | 260454 |
| 52437 | 93799229 | 93801004 | |
| 52438 | 93821993 | 93823730 | |
| 52439 | 93833762 | 93834863 | |
| 52440 | 93862139 | 93863919 | |
| 52441 | 93944579 | 93945085 | 229694 |
| 52442 | 93954462 | 93957194 | |
| 52443 | 93957214 | 93960863 | |
| 52444 | 93969418 | 93972085 | 273381 |
| 52445 | 93972318 | 93975141 | 273381 |
| 52446 | 94065037 | 94065453 | 148090 |
| 52447 | 94122857 | 94124654 | 148090 |
| 52448 | 94157209 | 94157973 | |
| 52449 | 94178592 | 94187862 | 165030 |
| 52450 | 94876879 | 94878337 | 090054 |
| 52451 | 94909363 | 94910426 | 225511 |
| 52452 | 94959033 | 94959682 | |
| 52453 | 95003951 | 95005565 | 196305 |
| 52454 | 95054505 | 95057221 | 239183, 196305 |
| 52455 | 95086594 | 95088684 | 188312, 198000 |
| 52456 | 95146789 | 95149272 | 188312, 106809 |
| 52457 | 95229315 | 95229944 | 188312, 106819 |
| 52458 | 95268229 | 95269698 | 106823, 188312 |
| 52459 | 95296957 | 95301339 | 225551, 106823, 188312 |
| 52460 | 95378059 | 95379847 | 188312, 127080 |
| 52461 | 95386890 | 95387417 | 127080 |
| 52462 | 95431196 | 95433519 | 127080 |
| 52463 | 95468546 | 95469106 | |
| 52464 | 95526185 | 95528168 | 185963 |
| 52465 | 95811264 | 95812378 | |
| 52466 | 95820325 | 95821736 | 157303 |
| 52467 | 95822892 | 95823761 | 157303 |
| 52468 | 95853659 | 95854409 | |
| 52469 | 95857053 | 95859751 | 223446, 165233 |
| 52470 | 95877895 | 95879426 | |
| 52471 | 95881556 | 95883431 | |
| 52472 | 95894530 | 95897627 | 131669 |
| 52473 | 96074979 | 96075780 | 165238 |
| 52474 | 96212815 | 96216279 | 048828, 188938 |
| 52475 | 96228870 | 96230157 | 048828 |
| 52476 | 96328525 | 96330134 | |
| 52477 | 96337592 | 96340671 | 197724 |
| 52478 | 96792382 | 96793864 | 158079 |
| 52479 | 96927836 | 96930292 | 269946 |
| 52480 | 97021358 | 97022337 | 175787 |
| 52481 | 97316302 | 97317596 | 231806 |
| 52482 | 97370236 | 97371284 | 165140 |
| 52483 | 97393677 | 97394402 | 165140 |
| 52484 | 97400455 | 97402903 | 165140 |
| 52485 | 97428024 | 97430321 | |
| 52486 | 97430790 | 97432017 | |
| 52487 | 97454627 | 97455524 | |
| 52488 | 97488521 | 97489932 | 148120 |
| 52489 | 97505586 | 97506367 | 148120 |
| 52490 | 97542663 | 97543287 | 148120 |
| 52491 | 97545034 | 97546432 | 148120 |
| 52492 | 97574600 | 97575052 | 148120 |
| 52493 | 97589597 | 97590656 | 148120 |
| 52494 | 97595995 | 97596779 | 148120 |
| 52495 | 97616058 | 97617193 | 148120 |
| 52496 | 97622820 | 97625378 | 148120 |
| 52497 | 97628472 | 97632294 | 148120 |
| 52498 | 97661968 | 97663652 | 236095, 148120 |
| 52499 | 97674358 | 97677523 | 148120 |
| 52500 | 97683585 | 97685859 | 148120 |
| 52501 | 97699373 | 97700323 | 148120 |
| 52502 | 97702698 | 97704763 | 148120 |
| 52503 | 97707049 | 97709092 | 148120 |
| 52504 | 97712970 | 97713933 | 148120 |
| 52505 | 97733060 | 97739083 | 148120 |
| 52506 | 97739217 | 97740308 | 148120 |
| 52507 | 97745572 | 97746315 | 148120 |
| 52508 | 97751818 | 97752948 | 148120 |
| 52509 | 97753151 | 97759450 | 148120 |
| 52510 | 97764890 | 97772175 | 148120 |
| 52511 | 97779696 | 97781699 | 148120 |
| 52512 | 97784847 | 97789110 | 148120 |
| 52513 | 97790181 | 97791095 | 148120 |
| 52514 | 97810459 | 97811465 | 148120 |
| 52515 | 97826482 | 97828663 | 148120 |
| 52516 | 97852330 | 97853064 | |
| 52517 | 97861579 | 97863747 | 158169 |
| 52518 | 97896314 | 97897408 | 158169 |
| 52519 | 97904569 | 97907365 | 251884, 158169 |
| 52520 | 97909631 | 97910486 | 158169 |
| 52521 | 97959597 | 97961002 | 158169 |
| 52522 | 98078975 | 98080432 | 158169 |
| 52523 | 98188225 | 98189976 | 237857 |
| 52524 | 98248318 | 98249085 | 185920 |
| 52525 | 98267936 | 98270840 | 271155, 185920 |
| 52526 | 98272931 | 98274105 | 185920 |
| 52527 | 98636833 | 98639286 | 182150, 175611 |
| 52528 | 98770740 | 98771623 | 182150 |
| 52529 | 98810262 | 98812176 | |
| 52530 | 99101202 | 99103052 | 130958 |
| 52531 | 99106232 | 99107418 | 130958 |
| 52532 | 99139641 | 99140372 | 130958 |
| 52533 | 99144650 | 99147467 | 130958 |
| 52534 | 99153190 | 99154872 | 165244 |
| 52535 | 99178790 | 99183951 | 165244 |
| 52536 | 99212206 | 99213733 | 130956 |
| 52537 | 99253023 | 99254793 | 130956, 081377 |
| 52538 | 99328255 | 99330146 | 081377 |
| 52539 | 99336741 | 99337691 | 081377 |
| 52540 | 99365843 | 99366753 | 081377 |
| 52541 | 99373054 | 99375471 | 081377 |
| 52542 | 99380365 | 99382259 | 081377 |
| 52543 | 99416584 | 99418297 | 158122 |
| 52544 | 99540035 | 99540681 | 081386 |
| 52545 | 99616025 | 99617177 | 196597 |
| 52546 | 99618552 | 99619676 | 196597 |
| 52547 | 99636394 | 99638591 | 196597 |
| 52548 | 99824615 | 99825250 | |
| 52549 | 100144327 | 100145604 | |
| 52550 | 100146839 | 100147441 | |
| 52551 | 100149081 | 100149901 | 228957 |
| 52552 | 100161177 | 100161849 | |
| 52553 | 100166901 | 100167459 | |
| 52554 | 100168760 | 100169365 | |
| 52555 | 100173853 | 100175159 | 196116 |
| 52556 | 100240525 | 100241228 | 196116 |
| 52557 | 100263312 | 100264865 | 136842 |
| 52558 | 100265970 | 100267676 | 136842 |
| 52559 | 100273028 | 100274181 | 136842 |
| 52560 | 100276768 | 100277323 | 228174, 136842 |
| 52561 | 100319843 | 100320497 | 136842 |
| 52562 | 100389784 | 100390485 | 136925 |
| 52563 | 100394111 | 100396914 | 136937, 231521, 136925 |
| 52564 | 100458506 | 100460450 | 136936 |
| 52565 | 100465290 | 100467059 | |
| 52566 | 100503317 | 100504098 | |
| 52567 | 100521172 | 100522881 | |
| 52568 | 100551308 | 100552244 | |
| 52569 | 100565490 | 100566208 | |
| 52570 | 100639020 | 100640035 | |
| 52571 | 100683811 | 100686457 | 136932 |
| 52572 | 100744454 | 100748079 | 136938 |
| 52573 | 100818065 | 100820158 | 095380 |
| 52574 | 100837071 | 100838900 | 095380, 106785 |
| 52575 | 100843041 | 100844316 | 095380, 106785 |
| 52576 | 100845362 | 100845860 | 106785 |
| 52577 | 100859352 | 100860551 | 106785 |
| 52578 | 100880247 | 100882149 | 106785 |
| 52579 | 100904149 | 100905767 | 106789 |
| 52580 | 100908197 | 100908882 | 106789 |
| 52581 | 100945248 | 100946077 | 106789 |
| 52582 | 100954133 | 100955960 | 106789 |
| 52583 | 100979048 | 100980052 | 095383 |
| 52584 | 100980779 | 100982909 | 095383 |
| 52585 | 100985183 | 100986008 | 095383 |
| 52586 | 100987330 | 100993802 | 095383 |
| 52587 | 101017250 | 101019417 | 095383 |
| 52588 | 101077687 | 101078438 | 136928 |
| 52589 | 101242180 | 101243095 | 136928 |
| 52590 | 101253076 | 101254474 | 136928 |
| 52591 | 101537013 | 101537820 | 165138 |
| 52592 | 101553352 | 101553862 | 165138 |
| 52593 | 101558015 | 101559577 | 165138 |
| 52594 | 101864704 | 101868638 | 106799 |
| 52595 | 101983142 | 101985712 | 106803, 119523 |
| 52596 | 102021021 | 102022447 | |
| 52597 | 102112100 | 102113743 | |
| 52598 | 102115523 | 102116718 | |
| 52599 | 102117942 | 102120604 | 271086 |
| 52600 | 102195708 | 102196971 | |
| 52601 | 102581161 | 102584570 | 119508, 237461 |
| 52602 | 102668187 | 102670004 | 136874, 255145 |
| 52603 | 102692717 | 102694598 | 136874 |
| 52604 | 102728919 | 102729517 | 136874 |
| 52605 | 102860391 | 102862962 | 119509, 023318 |
| 52606 | 102873384 | 102873991 | 119509 |
| 52607 | 103114081 | 103115556 | 136891 |
| 52608 | 103187990 | 103193111 | 066697 |
| 52609 | 103790538 | 103791579 | 148123 |
| 52610 | 103824806 | 103825728 | 148123 |
| 52611 | 103924443 | 103925105 | 148123 |
| 52612 | 103963335 | 103964424 | 148123 |
| 52613 | 104017259 | 104017896 | 148123 |
| 52614 | 104077392 | 104078620 | 148123 |
| 52615 | 104085404 | 104090251 | 148123 |
| 52616 | 104094395 | 104095446 | |
| 52617 | 104112483 | 104113107 | |
| 52618 | 104115372 | 104118410 | |
| 52619 | 104137567 | 104138964 | 234502, 136881 |
| 52620 | 104143303 | 104147605 | 136881 |
| 52621 | 104159708 | 104162078 | 136870, 136897 |
| 52622 | 104183911 | 104184522 | 136872 |
| 52623 | 104190080 | 104190905 | 136872 |
| 52624 | 104193805 | 104198703 | 136872 |
| 52625 | 104199212 | 104201693 | |
| 52626 | 104202553 | 104208709 | |
| 52627 | 104211155 | 104212092 | 270982 |
| 52628 | 104248224 | 104250187 | 165152 |
| 52629 | 104295192 | 104296996 | 155827, 165152 |
| 52630 | 104401491 | 104403090 | 198785 |
| 52631 | 104467323 | 104467987 | 198785 |
| 52632 | 104468422 | 104469658 | 198785 |
| 52633 | 104500314 | 104501326 | 198785 |
| 52634 | 104832184 | 104832867 | |
| 52635 | 104904653 | 104905242 | |
| 52636 | 105629769 | 105630384 | |
| 52637 | 106836895 | 106837675 | |
| 52638 | 106855558 | 106857209 | 136824 |
| 52639 | 107032497 | 107033411 | 242111 |
| 52640 | 107509297 | 107511284 | 136783 |
| 52641 | 107526027 | 107526967 | 165028 |
| 52642 | 107572530 | 107573831 | 165029 |
| 52643 | 107579092 | 107580362 | 165029 |
| 52644 | 107603797 | 107604382 | 165029 |
| 52645 | 107618485 | 107619319 | 165029 |
| 52646 | 107625352 | 107626093 | 165029 |
| 52647 | 107626205 | 107627706 | 165029 |
| 52648 | 107630578 | 107632057 | 165029 |
| 52649 | 107633414 | 107636929 | 165029 |
| 52650 | 107651578 | 107652742 | 165029 |
| 52651 | 107661469 | 107662893 | 165029 |
| 52652 | 107665288 | 107672612 | 165029 |
| 52653 | 107688066 | 107690931 | 226334, 165029 |
| 52654 | 107730360 | 107731436 | |
| 52655 | 107752984 | 107754795 | 230013 |
| 52656 | 107814797 | 107817488 | |
| 52657 | 107824120 | 107825398 | |
| 52658 | 107825483 | 107828144 | |
| 52659 | 107913544 | 107914038 | |
| 52660 | 107949975 | 107950493 | |
| 52661 | 107953427 | 107954794 | |
| 52662 | 107967951 | 107970312 | |
| 52663 | 108000588 | 108001269 | |
| 52664 | 108006046 | 108008330 | 070214 |
| 52665 | 108040955 | 108041559 | 070214 |
| 52666 | 108042311 | 108044632 | 070214 |
| 52667 | 108052898 | 108054721 | 070214 |
| 52668 | 108058488 | 108060135 | 070214 |
| 52669 | 108080830 | 108082550 | 070214 |
| 52670 | 108180580 | 108181427 | 070214 |
| 52671 | 108209910 | 108211202 | 106701 |
| 52672 | 108320206 | 108321141 | 106692, 228317 |
| 52673 | 108418222 | 108418974 | |
| 52674 | 108441442 | 108442760 | |
| 52675 | 108456525 | 108457977 | 095209 |
| 52676 | 108801470 | 108802164 | |
| 52677 | 109162396 | 109163913 | 234323 |
| 52678 | 109559201 | 109560637 | |
| 52679 | 109561253 | 109561885 | |
| 52680 | 109630556 | 109631732 | 148143 |
| 52681 | 109654920 | 109656848 | 148143 |
| 52682 | 109678471 | 109680116 | 148143 |
| 52683 | 109682840 | 109684271 | 148143 |
| 52684 | 109693789 | 109696233 | 148143 |
| 52685 | 109899786 | 109901346 | |
| 52686 | 109937008 | 109938439 | |
| 52687 | 109938710 | 109941975 | |
| 52688 | 109949474 | 109953789 | |
| 52689 | 109975519 | 109976492 | |
| 52690 | 109976888 | 109978131 | |
| 52691 | 110005789 | 110006719 | |
| 52692 | 110010729 | 110012481 | |
| 52693 | 110022259 | 110023451 | |
| 52694 | 110030776 | 110033383 | |
| 52695 | 110036813 | 110037655 | |
| 52696 | 110043241 | 110048860 | 119318 |
| 52697 | 110058187 | 110058915 | 119318 |
| 52698 | 110103138 | 110103924 | |
| 52699 | 110151456 | 110152547 | |
| 52700 | 110187157 | 110187932 | 226825 |
| 52701 | 110242668 | 110243192 | |
| 52702 | 110249684 | 110253153 | 136826 |
| 52703 | 110266270 | 110267674 | |
| 52704 | 110309030 | 110311847 | |
| 52705 | 110331138 | 110332253 | |
| 52706 | 110349308 | 110350840 | |
| 52707 | 110407160 | 110408158 | |
| 52708 | 110469760 | 110470818 | |
| 52709 | 110473568 | 110474359 | |
| 52710 | 110494976 | 110496303 | |
| 52711 | 110511564 | 110515865 | |
| 52712 | 110516811 | 110518156 | |
| 52713 | 110661480 | 110662104 | |
| 52714 | 110751397 | 110752220 | |
| 52715 | 110800873 | 110805058 | 213557 |
| 52716 | 110855851 | 110857632 | |
| 52717 | 110870720 | 110871900 | |
| 52718 | 111229375 | 111230958 | |
| 52719 | 111231824 | 111232490 | |
| 52720 | 111235366 | 111236544 | |
| 52721 | 111314693 | 111315277 | |
| 52722 | 111342239 | 111342944 | |
| 52723 | 111509197 | 111509768 | |
| 52724 | 111631151 | 111632236 | 070061 |
| 52725 | 111647657 | 111649508 | 070061 |
| 52726 | 111695238 | 111697564 | 119328, 070061 |
| 52727 | 111763518 | 111764113 | 119326 |
| 52728 | 111773925 | 111776379 | 119326 |
| 52729 | 111797273 | 111800019 | 106771 |
| 52730 | 111876276 | 111877430 | 106771 |
| 52731 | 111879521 | 111882723 | 106771 |
| 52732 | 112009670 | 112011069 | 095203 |
| 52733 | 112012639 | 112018133 | 095203 |
| 52734 | 112039029 | 112040507 | 095203 |
| 52735 | 112069536 | 112070210 | 095203 |
| 52736 | 112075528 | 112076775 | 095203 |
| 52737 | 112078265 | 112079205 | 095203 |
| 52738 | 112081212 | 112082953 | 095203 |
| 52739 | 112083019 | 112084186 | 095203 |
| 52740 | 112202845 | 112204073 | 070159 |
| 52741 | 112214544 | 112216739 | 070159 |
| 52742 | 112237769 | 112242053 | 070159 |
| 52743 | 112259722 | 112261487 | 070159 |
| 52744 | 112261574 | 112262886 | |
| 52745 | 112268342 | 112268824 | |
| 52746 | 112268926 | 112270721 | |
| 52747 | 112280151 | 112280802 | |
| 52748 | 112365071 | 112365842 | |
| 52749 | 112395035 | 112395547 | |
| 52750 | 112402322 | 112403472 | 243444 |
| 52751 | 112461302 | 112461837 | 243444 |
| 52752 | 112467723 | 112468522 | 243444 |
| 52753 | 112495857 | 112496648 | 243444 |
| 52754 | 112502018 | 112502592 | 243444 |
| 52755 | 112528003 | 112529006 | 243444, 232939 |
| 52756 | 112541557 | 112544644 | 157654, 241978, 243444 |
| 52757 | 112553152 | 112558579 | 157654, 243444, 241978 |
| 52758 | 112568754 | 112569663 | 157654, 243444, 241978 |
| 52759 | 112573977 | 112581324 | 157654, 243444, 241978 |
| 52760 | 112664513 | 112666506 | 157654, 243444, 241978 |
| 52761 | 112669239 | 112670947 | 157654, 243444, 241978 |
| 52762 | 112672649 | 112674423 | 157654, 243444, 241978 |
| 52763 | 112683794 | 112686299 | 157654, 243444, 241978 |
| 52764 | 112687510 | 112689267 | 157654, 243444, 241978 |
| 52765 | 112736163 | 112736876 | 157654, 241978 |
| 52766 | 112748361 | 112749608 | 157654, 241978 |
| 52767 | 112753708 | 112756258 | 157654, 241978 |
| 52768 | 112777866 | 112779586 | 157654, 241978 |
| 52769 | 112788591 | 112796825 | 157654, 241978 |
| 52770 | 112801504 | 112804345 | 157654, 241978 |
| 52771 | 112805807 | 112807439 | 157654, 241978 |
| 52772 | 112811544 | 112815355 | 157654, 241978 |
| 52773 | 112821422 | 112822344 | 157654, 241978 |
| 52774 | 112835661 | 112836340 | 157654, 241978 |
| 52775 | 112877666 | 112879388 | 157654, 241978 |
| 52776 | 112885170 | 112889908 | 157654, 241978 |
| 52777 | 112902798 | 112905490 | 157654, 241978 |
| 52778 | 112956319 | 112957003 | 188959 |
| 52779 | 112975615 | 112976638 | |
| 52780 | 112982825 | 112983704 | |
| 52781 | 113004715 | 113005360 | |
| 52782 | 113016787 | 113020710 | 136810 |
| 52783 | 113040308 | 113040714 | |
| 52784 | 113318112 | 113318748 | 165124 |
| 52785 | 114243989 | 114247384 | 136813 |
| 52786 | 114320409 | 114322500 | 173258, 106853 |
| 52787 | 114323506 | 114324506 | 173258, 106853 |
| 52788 | 114347768 | 114349126 | 106853 |
| 52789 | 114351049 | 114351924 | 106853 |
| 52790 | 114359226 | 114365829 | 106853, 204173 |
| 52791 | 114366460 | 114368416 | 204173 |
| 52792 | 114375399 | 114376361 | 204173 |
| 52793 | 114384111 | 114385845 | 204173 |
| 52794 | 114388930 | 114390353 | 204173 |
| 52795 | 114392635 | 114395210 | 244115, 059769, 204173 |
| 52796 | 114395355 | 114396274 | 244115, 059769 |
| 52797 | 114397018 | 114397751 | 244115, 059769 |
| 52798 | 114408662 | 114409101 | 244115, 059769 |
| 52799 | 114423469 | 114424900 | 242616, 244115, 059769 |
| 52800 | 114440307 | 114441175 | |
| 52801 | 114560917 | 114561544 | |
| 52802 | 114658140 | 114661241 | 148154 |
| 52803 | 114679314 | 114680614 | 148154 |
| 52804 | 114721065 | 114724781 | |
| 52805 | 114726920 | 114730195 | |
| 52806 | 114773783 | 114776247 | |
| 52807 | 114782407 | 114784993 | |
| 52808 | 114792130 | 114792889 | |
| 52809 | 114795434 | 114796398 | 259953 |
| 52810 | 114803595 | 114805400 | 106868 |
| 52811 | 114811446 | 114813491 | 106868 |
| 52812 | 114820994 | 114824760 | 106868 |
| 52813 | 114827125 | 114830049 | 106868 |
| 52814 | 114830924 | 114833342 | 106868 |
| 52815 | 114836653 | 114837358 | 106868 |
| 52816 | 114880952 | 114882879 | 106868 |
| 52817 | 114888140 | 114888853 | 106868 |
| 52818 | 114936739 | 114938350 | 106868 |
| 52819 | 115033837 | 115034934 | 243911, 119314 |
| 52820 | 115061646 | 115062673 | 119314 |
| 52821 | 115094563 | 115097459 | 119314 |
| 52822 | 115099164 | 115100628 | |
| 52823 | 115103336 | 115104866 | |
| 52824 | 115109925 | 115112351 | |
| 52825 | 115140218 | 115143476 | 119471 |
| 52826 | 115248322 | 115250337 | 165185, 230185 |
| 52827 | 115293603 | 115295060 | 165185 |
| 52828 | 115318268 | 115319135 | 165185 |
| 52829 | 115332873 | 115333737 | 165185 |
| 52830 | 115479364 | 115480831 | 148153 |
| 52831 | 115494054 | 115494970 | |
| 52832 | 115512131 | 115514318 | 148158, 226609 |
| 52833 | 115579354 | 115580132 | 148158 |
| 52834 | 115598602 | 115599356 | 148158 |
| 52835 | 115818339 | 115819873 | 136866 |
| 52836 | 115899674 | 115900508 | |
| 52837 | 115912829 | 115914562 | 136867 |
| 52838 | 115914824 | 115915492 | 136867 |
| 52839 | 115948864 | 115951711 | 119321 |
| 52840 | 115982521 | 115985697 | 136868, 119321 |
| 52841 | 116000568 | 116001720 | 136868 |
| 52842 | 116024073 | 116025248 | 176386, 136868 |
| 52843 | 116036637 | 116038905 | 136875, 176386 |
| 52844 | 116102306 | 116102981 | 148225 |
| 52845 | 116131643 | 116132344 | 119411 |
| 52846 | 116137235 | 116140155 | 119431 |
| 52847 | 116144201 | 116144827 | |
| 52848 | 116149795 | 116150555 | 148218 |
| 52849 | 116151006 | 116151622 | 148218 |
| 52850 | 116157374 | 116158162 | 148218 |
| 52851 | 116160192 | 116161402 | 148218 |
| 52852 | 116163095 | 116164097 | 148218 |
| 52853 | 116171626 | 116174005 | 157653, 148229 |
| 52854 | 116217019 | 116218421 | 138835 |
| 52855 | 116225763 | 116226542 | 138835 |
| 52856 | 116266707 | 116268171 | 138835 |
| 52857 | 116273126 | 116273966 | 138835 |
| 52858 | 116281706 | 116283020 | 138835 |
| 52859 | 116310131 | 116310931 | 138835 |
| 52860 | 116321160 | 116321908 | 138835 |
| 52861 | 116333324 | 116333904 | 138835, 237073 |
| 52862 | 116342525 | 116344677 | 138835, 237073 |
| 52863 | 116355893 | 116358299 | 138835 |
| 52864 | 116364305 | 116365421 | |
| 52865 | 116371616 | 116372874 | |
| 52866 | 116377189 | 116377714 | |
| 52867 | 116381560 | 116384163 | 233817 |
| 52868 | 116384630 | 116386727 | 233817 |
| 52869 | 116412219 | 116413459 | 227482 |
| 52870 | 116413584 | 116415816 | 227482 |
| 52871 | 116417548 | 116417965 | 227482 |
| 52872 | 116444404 | 116445052 | 227482 |
| 52873 | 116468092 | 116469038 | |
| 52874 | 116474071 | 116474796 | |
| 52875 | 116615514 | 116616081 | |
| 52876 | 116637713 | 116640079 | 157657 |
| 52877 | 116668229 | 116670087 | 157657 |
| 52878 | 116672378 | 116672922 | 157657 |
| 52879 | 116727968 | 116729415 | 157657 |
| 52880 | 116751308 | 116752172 | 157657 |
| 52881 | 116754280 | 116755404 | 157657 |
| 52882 | 116838990 | 116841774 | 106927 |
| 52883 | 116842048 | 116845249 | |
| 52884 | 116860984 | 116862428 | 136883 |
| 52885 | 116872416 | 116873052 | |
| 52886 | 116880370 | 116881284 | |
| 52887 | 116915842 | 116916606 | |
| 52888 | 116917249 | 116919318 | 196739 |
| 52889 | 116925457 | 116927161 | 196739 |
| 52890 | 116936408 | 116939588 | 196739 |
| 52891 | 116939937 | 116940475 | 196739 |
| 52892 | 116985286 | 116987118 | 196739 |
| 52893 | 117050360 | 117051228 | 196739 |
| 52894 | 117069586 | 117071594 | 196739 |
| 52895 | 117076548 | 117079657 | |
| 52896 | 117079699 | 117081148 | |
| 52897 | 117090061 | 117091952 | |
| 52898 | 117110754 | 117112395 | 106948 |
| 52899 | 117126317 | 117127437 | 106948 |
| 52900 | 117128731 | 117129305 | 106948 |
| 52901 | 117147199 | 117148991 | 106948 |
| 52902 | 117154632 | 117155320 | 106948 |
| 52903 | 117159814 | 117160983 | 235119 |
| 52904 | 117255642 | 117256510 | 095397 |
| 52905 | 117267048 | 117268223 | 095397 |
| 52906 | 117348967 | 117352222 | 136888 |
| 52907 | 117353373 | 117354636 | 136888 |
| 52908 | 117368097 | 117369567 | |
| 52909 | 117372914 | 117374755 | 157693 |
| 52910 | 117384175 | 117384665 | 157693 |
| 52911 | 117483767 | 117484618 | |
| 52912 | 117540863 | 117542236 | |
| 52913 | 117646850 | 117648146 | |
| 52914 | 117655036 | 117658666 | 106952 |
| 52915 | 117670054 | 117671669 | 106952 |
| 52916 | 117673198 | 117673949 | 106952 |
| 52917 | 117715951 | 117716653 | |
| 52918 | 117764755 | 117765433 | |
| 52919 | 117867447 | 117870461 | 041982 |
| 52920 | 117918791 | 117920266 | 173077 |
| 52921 | 117921204 | 117921794 | 173077 |
| 52922 | 117971161 | 117977381 | 173077 |
| 52923 | 117996020 | 117996993 | 173077 |
| 52924 | 118135138 | 118136987 | 173077 |
| 52925 | 118257124 | 118258216 | |
| 52926 | 118353736 | 118354355 | |
| 52927 | 118412181 | 118412777 | |
| 52928 | 118703666 | 118709591 | |
| 52929 | 118758672 | 118759219 | |
| 52930 | 118764594 | 118765392 | |
| 52931 | 118937853 | 118938467 | 182752 |
| 52932 | 118964590 | 118965006 | 182752 |
| 52933 | 118966246 | 118967096 | 182752 |
| 52934 | 119308656 | 119314453 | 148219, 229105 |
| 52935 | 119328556 | 119331698 | 230894, 148219 |
| 52936 | 119336254 | 119337061 | 148219, 230894 |
| 52937 | 119448663 | 119450613 | 119401, 148219 |
| 52938 | 120249662 | 120250830 | |
| 52939 | 120253314 | 120254388 | |
| 52940 | 120406381 | 120407604 | |
| 52941 | 120424585 | 120425419 | |
| 52942 | 120434553 | 120435130 | |
| 52943 | 120482740 | 120483866 | |
| 52944 | 121067363 | 121070421 | |
| 52945 | 121080054 | 121082353 | |
| 52946 | 121092669 | 121100866 | |
| 52947 | 121216609 | 121218737 | |
| 52948 | 121339036 | 121340124 | |
| 52949 | 121357871 | 121359566 | |
| 52950 | 121402836 | 121404148 | |
| 52951 | 121405182 | 121406298 | |
| 52952 | 121512633 | 121515891 | |
| 52953 | 121755591 | 121756126 | |
| 52954 | 121765956 | 121767881 | |
| 52955 | 122318253 | 122318893 | |
| 52956 | 123128753 | 123129459 | |
| 52957 | 123145851 | 123147779 | |
| 52958 | 123148318 | 123149684 | |
| 52959 | 123149798 | 123150406 | |
| 52960 | 123152870 | 123153717 | 136861 |
| 52961 | 123155518 | 123157149 | 136861 |
| 52962 | 123165389 | 123166505 | 136861 |
| 52963 | 123242958 | 123243914 | 136861 |
| 52964 | 123284330 | 123285100 | 136861 |
| 52965 | 123300315 | 123301194 | 136861 |
| 52966 | 123333090 | 123334776 | 136861 |
| 52967 | 123341537 | 123343233 | 136861 |
| 52968 | 123346039 | 123346733 | |
| 52969 | 123379526 | 123380866 | 106780 |
| 52970 | 123402551 | 123403132 | 106780 |
| 52971 | 123409159 | 123410014 | 106780 |
| 52972 | 123446037 | 123447551 | 106780 |
| 52973 | 123474503 | 123478201 | 106780 |
| 52974 | 123488209 | 123488701 | |
| 52975 | 123491868 | 123494790 | |
| 52976 | 123495101 | 123496368 | |
| 52977 | 123497007 | 123498477 | |
| 52978 | 123502392 | 123502780 | |
| 52979 | 123505200 | 123505661 | |
| 52980 | 123540851 | 123541577 | 119402 |
| 52981 | 123545814 | 123546470 | 119402 |
| 52982 | 123554742 | 123556601 | 214654, 119402 |
| 52983 | 123604395 | 123606028 | 095261, 226752 |
| 52984 | 123638136 | 123640120 | 119403 |
| 52985 | 123655373 | 123657594 | |
| 52986 | 123658177 | 123660009 | |
| 52987 | 123675828 | 123676403 | 056558 |
| 52988 | 123685402 | 123692260 | 056558 |
| 52989 | 123693015 | 123695144 | |
| 52990 | 123697536 | 123699319 | |
| 52991 | 123704378 | 123705304 | |
| 52992 | 123744937 | 123745754 | 106804 |
| 52993 | 123788493 | 123789725 | 106804 |
| 52994 | 123812145 | 123813129 | 106804 |
| 52995 | 123813957 | 123814685 | |
| 52996 | 123815369 | 123816273 | |
| 52997 | 123817668 | 123818482 | |
| 52998 | 123836405 | 123837835 | 119397 |
| 52999 | 123883700 | 123884327 | 119397 |
| 53000 | 123962894 | 123964886 | 119396 |
| 53001 | 124043514 | 124044861 | 148180, 239593, 235865 |
| 53002 | 124048658 | 124049925 | 148180 |
| 53003 | 124079759 | 124080553 | 148180 |
| 53004 | 124082198 | 124086347 | 148180 |
| 53005 | 124086798 | 124090176 | 148180 |
| 53006 | 124124938 | 124125693 | 148175 |
| 53007 | 124129689 | 124133229 | 148175 |
| 53008 | 124134468 | 124136528 | |
| 53009 | 124261901 | 124262331 | 204136 |
| 53010 | 124360516 | 124362886 | 136848 |
| 53011 | 124369683 | 124370246 | 136848 |
| 53012 | 124371945 | 124372864 | 136848 |
| 53013 | 124374507 | 124375379 | 136848 |
| 53014 | 124384325 | 124385482 | 136848 |
| 53015 | 124400067 | 124400494 | 136848 |
| 53016 | 124412499 | 124414332 | 136848 |
| 53017 | 124416826 | 124417384 | 136848 |
| 53018 | 124505668 | 124507582 | 136848 |
| 53019 | 124535854 | 124536388 | 136848 |
| 53020 | 124604756 | 124605293 | 175764 |
| 53021 | 124623675 | 124624217 | 175764 |
| 53022 | 124734748 | 124740159 | 175764 |
| 53023 | 124760745 | 124761700 | 175764 |
| 53024 | 124763369 | 124764962 | 175764 |
| 53025 | 124854501 | 124856045 | 175764 |
| 53026 | 124921322 | 124922251 | 185681, 119421 |
| 53027 | 124952721 | 124953441 | 185681 |
| 53028 | 125025836 | 125028548 | 148187, 119446 |
| 53029 | 125087401 | 125087980 | 269979 |
| 53030 | 125108520 | 125109977 | |
| 53031 | 125540085 | 125541090 | |
| 53032 | 125590103 | 125591453 | 136940 |
| 53033 | 125666586 | 125668073 | 056586 |
| 53034 | 125674602 | 125676458 | 186130 |
| 53035 | 125693009 | 125694685 | 171448 |
| 53036 | 125703001 | 125704562 | 011454 |
| 53037 | 125774397 | 125774806 | 011454 |
| 53038 | 125811971 | 125812547 | 011454 |
| 53039 | 125821059 | 125821500 | 011454 |
| 53040 | 125849483 | 125850186 | 011454 |
| 53041 | 125861658 | 125863365 | 011454 |
| 53042 | 125864817 | 125865633 | 011454 |
| 53043 | 125871361 | 125872535 | 236901, 165209 |
| 53044 | 126029755 | 126031038 | 165209 |
| 53045 | 126086946 | 126087587 | |
| 53046 | 126227196 | 126228139 | 119522 |
| 53047 | 126229766 | 126230458 | 119522 |
| 53048 | 126232065 | 126233145 | 119522 |
| 53049 | 126241158 | 126242316 | 119522 |
| 53050 | 126246014 | 126247094 | 119522 |
| 53051 | 126300125 | 126300618 | 119522 |
| 53052 | 126304460 | 126305203 | 119522 |
| 53053 | 126369669 | 126370374 | 119522 |
| 53054 | 126413530 | 126414057 | 119522 |
| 53055 | 126523962 | 126524879 | 119522 |
| 53056 | 126629599 | 126631298 | 119522 |
| 53057 | 126683890 | 126685453 | 119522 |
| 53058 | 126691111 | 126692862 | 119522 |
| 53059 | 126695581 | 126697058 | |
| 53060 | 126758926 | 126759370 | |
| 53061 | 126802956 | 126803911 | |
| 53062 | 126807338 | 126808410 | |
| 53063 | 126839939 | 126841967 | |
| 53064 | 126906975 | 126907712 | |
| 53065 | 126979503 | 126981861 | |
| 53066 | 126982501 | 126984938 | |
| 53067 | 126985170 | 126986269 | |
| 53068 | 127019586 | 127024700 | 119408, 235204 |
| 53069 | 127025967 | 127027058 | 119408, 227200 |
| 53070 | 127029017 | 127030585 | 119408 |
| 53071 | 127033154 | 127033931 | 119408 |
| 53072 | 127041780 | 127043004 | 119408 |
| 53073 | 127044326 | 127050649 | 119408 |
| 53074 | 127059332 | 127060315 | 119408 |
| 53075 | 127071904 | 127072882 | 119408 |
| 53076 | 127147933 | 127149343 | 136930 |
| 53077 | 127152916 | 127153621 | 136930 |
| 53078 | 127176500 | 127178162 | 136930 |
| 53079 | 127272879 | 127273533 | |
| 53080 | 127313749 | 127315071 | 148200 |
| 53081 | 127315209 | 127317063 | 148200 |
| 53082 | 127357444 | 127358901 | 148200 |
| 53083 | 127379634 | 127381393 | 148200 |
| 53084 | 127420336 | 127423092 | 224020, 148200 |
| 53085 | 127486779 | 127487879 | 148200 |
| 53086 | 127531212 | 127534872 | 236643, 148200 |
| 53087 | 127539218 | 127540120 | 185585 |
| 53088 | 127580354 | 127581546 | |
| 53089 | 127614955 | 127615964 | 136918 |
| 53090 | 127619276 | 127620003 | 136918 |
| 53091 | 127622541 | 127624988 | 136950, 136942 |
| 53092 | 127630107 | 127633199 | 136950 |
| 53093 | 127702433 | 127704860 | 136935 |
| 53094 | 127709946 | 127710625 | 136935, 173611 |
| 53095 | 127712123 | 127713188 | 173611 |
| 53096 | 127904992 | 127906329 | 173611 |
| 53097 | 127912585 | 127913475 | 232630, 119414 |
| 53098 | 127951133 | 127953050 | 119414 |
| 53099 | 127962025 | 127963783 | 136933 |
| 53100 | 128002364 | 128005308 | 239705, 044574 |
| 53101 | 128005495 | 128006475 | 239705 |
| 53102 | 128023467 | 128025306 | 165219 |
| 53103 | 128034862 | 128035447 | 165219 |
| 53104 | 128059113 | 128059853 | 165219 |
| 53105 | 128091278 | 128091882 | 165219 |
| 53106 | 128170117 | 128171351 | |
| 53107 | 128190140 | 128191090 | |
| 53108 | 128222280 | 128224308 | 119487 |
| 53109 | 128230757 | 128231618 | 119487 |
| 53110 | 128237709 | 128238304 | 119487 |
| 53111 | 128246922 | 128249444 | 119487 |
| 53112 | 128255047 | 128257665 | 119487 |
| 53113 | 128275227 | 128277000 | 243845, 119487 |
| 53114 | 128299578 | 128301563 | 119487 |
| 53115 | 128341217 | 128341984 | 119487 |
| 53116 | 128347617 | 128348662 | 119487 |
| 53117 | 128393517 | 128396793 | 119487 |
| 53118 | 128436522 | 128438717 | 119487 |
| 53119 | 128468263 | 128470310 | 119487 |
| 53120 | 128508425 | 128511473 | 167081, 229582 |
| 53121 | 128558038 | 128558973 | 167081 |
| 53122 | 128908258 | 128909780 | 232413 |
| 53123 | 128912791 | 128914463 | 232413 |
| 53124 | 129089131 | 129090184 | 196814 |
| 53125 | 129096450 | 129099398 | 196814 |
| 53126 | 129157610 | 129158546 | 196814 |
| 53127 | 129183146 | 129183852 | 196814 |
| 53128 | 129553714 | 129554803 | |
| 53129 | 129564541 | 129565420 | |
| 53130 | 129566979 | 129569289 | 169155 |
| 53131 | 129622269 | 129623826 | 177125 |
| 53132 | 129647351 | 129647962 | 177125 |
| 53133 | 129920857 | 129923327 | 136828 |
| 53134 | 129957527 | 129958262 | 136828 |
| 53135 | 129986768 | 129987225 | 136895 |
| 53136 | 129987278 | 129988447 | 136895 |
| 53137 | 130007007 | 130008826 | 136895 |
| 53138 | 130065983 | 130066487 | 136895 |
| 53139 | 130069334 | 130072397 | 136895 |
| 53140 | 130073429 | 130074900 | 136895 |
| 53141 | 130098420 | 130100231 | 136895 |
| 53142 | 130158803 | 130161462 | 136856 |
| 53143 | 130185575 | 130187792 | 196152 |
| 53144 | 130207610 | 130208072 | 196152 |
| 53145 | 130212347 | 130215392 | 148356, 197958 |
| 53146 | 130307513 | 130308096 | 136830 |
| 53147 | 130330290 | 130331457 | 136830 |
| 53148 | 130331503 | 130332316 | 136830 |
| 53149 | 130399677 | 130400255 | 136854 |
| 53150 | 130461339 | 130461838 | 187024 |
| 53151 | 130477190 | 130478290 | 160401, 187024 |
| 53152 | 130496627 | 130498001 | 160404 |
| 53153 | 130502219 | 130503582 | 095370 |
| 53154 | 130546992 | 130549538 | 266070, 136807 |
| 53155 | 130550397 | 130552328 | 136807 |
| 53156 | 130555118 | 130557331 | 136877 |
| 53157 | 130557802 | 130559399 | 136877 |
| 53158 | 130563213 | 130563695 | 136877 |
| 53159 | 130564381 | 130567156 | 136877 |
| 53160 | 130590463 | 130591209 | 106991 |
| 53161 | 130600142 | 130602022 | 106991 |
| 53162 | 130639127 | 130640850 | 106992, 257524 |
| 53163 | 130659376 | 130659850 | 160408 |
| 53164 | 130660667 | 130662193 | 160408 |
| 53165 | 130678610 | 130679491 | 136840 |
| 53166 | 130680410 | 130682200 | |
| 53167 | 130699343 | 130701080 | 136908, 227218 |
| 53168 | 130714202 | 130715177 | 167106 |
| 53169 | 130716129 | 130718964 | 167106 |
| 53170 | 130724591 | 130732445 | 167106 |
| 53171 | 130734389 | 130736728 | 167106 |
| 53172 | 130739226 | 130744515 | 167106 |
| 53173 | 130748585 | 130752221 | |
| 53174 | 130752698 | 130753859 | |
| 53175 | 130763478 | 130764544 | |
| 53176 | 130797179 | 130797926 | |
| 53177 | 130817663 | 130820625 | 230848 |
| 53178 | 130828530 | 130832053 | 148339, 171169 |
| 53179 | 130834396 | 130835168 | 148339 |
| 53180 | 130835201 | 130836012 | 148339 |
| 53181 | 130859802 | 130867308 | 148339 |
| 53182 | 130877016 | 130878415 | 234771 |
| 53183 | 130889399 | 130891398 | 232850, 148334 |
| 53184 | 130911051 | 130912750 | 148346 |
| 53185 | 130953117 | 130955562 | 148337 |
| 53186 | 131037409 | 131039618 | 175854, 167110 |
| 53187 | 131057477 | 131059892 | |
| 53188 | 131083729 | 131086369 | 167113, 167112 |
| 53189 | 131101660 | 131104047 | 167114 |
| 53190 | 131132403 | 131134741 | 167118 |
| 53191 | 131218310 | 131219176 | 136811 |
| 53192 | 131266170 | 131267775 | 119392 |
| 53193 | 131285083 | 131286390 | 119392 |
| 53194 | 131313830 | 131316120 | 197694, 228395 |
| 53195 | 131317113 | 131317960 | 197694 |
| 53196 | 131319372 | 131319994 | 197694 |
| 53197 | 131320761 | 131321646 | 197694 |
| 53198 | 131369725 | 131370494 | 197694 |
| 53199 | 131396836 | 131399043 | 119333 |
| 53200 | 131418469 | 131419859 | 119333 |
| 53201 | 131445445 | 131447458 | 119335 |
| 53202 | 131450445 | 131453186 | 119335 |
| 53203 | 131464566 | 131465567 | 160447 |
| 53204 | 131486042 | 131487163 | 223478, 160446 |
| 53205 | 131524573 | 131525928 | 160445 |
| 53206 | 131527267 | 131528202 | 160445 |
| 53207 | 131532683 | 131535159 | 160445 |
| 53208 | 131548948 | 131550334 | 107021 |
| 53209 | 131580318 | 131581778 | 167136 |
| 53210 | 131591098 | 131592230 | 198917 |
| 53211 | 131624771 | 131625325 | 171097 |
| 53212 | 131636543 | 131638939 | 171097 |
| 53213 | 131639362 | 131646070 | 136802, 171097 |
| 53214 | 131654070 | 131655758 | 136802 |
| 53215 | 131673117 | 131676829 | 136802 |
| 53216 | 131677645 | 131680259 | 136802 |
| 53217 | 131681191 | 131683585 | 175287 |
| 53218 | 131683627 | 131685418 | 175287 |
| 53219 | 131708833 | 131711018 | 095319, 175283, 251184 |
| 53220 | 131789747 | 131790614 | 148341 |
| 53221 | 131798299 | 131799443 | 148343 |
| 53222 | 131826653 | 131827308 | 148343 |
| 53223 | 131831146 | 131832562 | 148343 |
| 53224 | 131838307 | 131838754 | |
| 53225 | 131842812 | 131844340 | 167130 |
| 53226 | 131851492 | 131852077 | 167130 |
| 53227 | 131871768 | 131874696 | 119383, 095321 |
| 53228 | 131900076 | 131904552 | 119383 |
| 53229 | 131908013 | 131908712 | 119383 |
| 53230 | 131935881 | 131941142 | 188483, 204055, 268050 |
| 53231 | 132077169 | 132078798 | |
| 53232 | 132085566 | 132086975 | 179082 |
| 53233 | 132089756 | 132090486 | |
| 53234 | 132095580 | 132097742 | 233901 |
| 53235 | 132144959 | 132146529 | |
| 53236 | 132174800 | 132177542 | |
| 53237 | 132199444 | 132199950 | 230676 |
| 53238 | 132221384 | 132223814 | |
| 53239 | 132238960 | 132240093 | |
| 53240 | 132243747 | 132247264 | 204054, 261334 |
| 53241 | 132247658 | 132254151 | 204054 |
| 53242 | 132255078 | 132256211 | 204054 |
| 53243 | 132256243 | 132260737 | 204054 |
| 53244 | 132269086 | 132269672 | 239353, 204054 |
| 53245 | 132320821 | 132321707 | |
| 53246 | 132323011 | 132324541 | |
| 53247 | 132330559 | 132331916 | |
| 53248 | 132359138 | 132360277 | |
| 53249 | 132371629 | 132373922 | 148335 |
| 53250 | 132388118 | 132390316 | 148335 |
| 53251 | 132403225 | 132405045 | 234789, 148331 |
| 53252 | 132499753 | 132500846 | 148344, 255824 |
| 53253 | 132565049 | 132566398 | 136816 |
| 53254 | 132573042 | 132574176 | 136816 |
| 53255 | 132585267 | 132587051 | 136827 |
| 53256 | 132596693 | 132598744 | 136878, 136819 |
| 53257 | 132647215 | 132648701 | |
| 53258 | 132743236 | 132744485 | 187239 |
| 53259 | 132772390 | 132773978 | 187239 |
| 53260 | 132778872 | 132779693 | 187239 |
| 53261 | 132784778 | 132788393 | 187239 |
| 53262 | 132789565 | 132791509 | 187239 |
| 53263 | 132793161 | 132793914 | 187239 |
| 53264 | 132803932 | 132806599 | 187239 |
| 53265 | 132815356 | 132817183 | 148358 |
| 53266 | 132879376 | 132881195 | 148358 |
| 53267 | 133004909 | 133005889 | |
| 53268 | 133025574 | 133026920 | |
| 53269 | 133307634 | 133309916 | 148357 |
| 53270 | 133319424 | 133323267 | 130707 |
| 53271 | 133325514 | 133327933 | 130707 |
| 53272 | 133333915 | 133335729 | 130707 |
| 53273 | 133341616 | 133342658 | 130707 |
| 53274 | 133373627 | 133376459 | 130707 |
| 53275 | 133402304 | 133402886 | |
| 53276 | 133406299 | 133406738 | |
| 53277 | 133453620 | 133456047 | 107164 |
| 53278 | 133472482 | 133474114 | 107164 |
| 53279 | 133556055 | 133557857 | 130711 |
| 53280 | 133568249 | 133569900 | 130713 |
| 53281 | 133587731 | 133589457 | 097007 |
| 53282 | 133615274 | 133616524 | 097007 |
| 53283 | 133616880 | 133618450 | 097007 |
| 53284 | 133709409 | 133711668 | 097007 |
| 53285 | 133722053 | 133723602 | 097007 |
| 53286 | 133733682 | 133734118 | 097007 |
| 53287 | 133747947 | 133749008 | 097007 |
| 53288 | 133803319 | 133804874 | 130720 |
| 53289 | 134000022 | 134001953 | 126883 |
| 53290 | 134192489 | 134192972 | |
| 53291 | 134225151 | 134225904 | |
| 53292 | 134233148 | 134233722 | |
| 53293 | 134258528 | 134259086 | |
| 53294 | 134268303 | 134270824 | 130723 |
| 53295 | 134277620 | 134278755 | 130723 |
| 53296 | 134297635 | 134298784 | 130723 |
| 53297 | 134322618 | 134323567 | 130723 |
| 53298 | 134378133 | 134379406 | 130714 |
| 53299 | 134404979 | 134407380 | 130717 |
| 53300 | 134420516 | 134421319 | |
| 53301 | 134466323 | 134467316 | 107263 |
| 53302 | 134481086 | 134482298 | 107263 |
| 53303 | 134483519 | 134484470 | 107263 |
| 53304 | 134510045 | 134511546 | 107263 |
| 53305 | 134525118 | 134527418 | 107263 |
| 53306 | 134529998 | 134531113 | 107263 |
| 53307 | 134533179 | 134534702 | 107263 |
| 53308 | 134567422 | 134568477 | 107263 |
| 53309 | 134580920 | 134584528 | 107263 |
| 53310 | 134593169 | 134593953 | 107263 |
| 53311 | 134606277 | 134612204 | 107263 |
| 53312 | 134614110 | 134615954 | 107263 |
| 53313 | 134954187 | 134955587 | 160563 |
| 53314 | 135039804 | 135040964 | 196358 |
| 53315 | 135089308 | 135090040 | 196358 |
| 53316 | 135144075 | 135146755 | 107290 |
| 53317 | 135229146 | 135231085 | 107290 |
| 53318 | 135281821 | 135282521 | 125482 |
| 53319 | 135544415 | 135546986 | 125484, 125485 |
| 53320 | 135644340 | 135646238 | 224992, 165695 |
| 53321 | 135753447 | 135754401 | 165695, 165698 |
| 53322 | 135818962 | 135821027 | 165702, 165699 |
| 53323 | 135821790 | 135822493 | 233800, 165702 |
| 53324 | 135904883 | 135907078 | 148308 |
| 53325 | 135988080 | 135990196 | 160271 |
| 53326 | 135995761 | 135997041 | 160271 |
| 53327 | 136001444 | 136003767 | 160271 |
| 53328 | 136008915 | 136010468 | 160271 |
| 53329 | 136019059 | 136020749 | 160271 |
| 53330 | 136023786 | 136025331 | 160271 |
| 53331 | 136202186 | 136203923 | 148296 |
| 53332 | 136213304 | 136216260 | 148303, 206611, 148297 |
| 53333 | 136222383 | 136224388 | 148291, 148290 |
| 53334 | 136241441 | 136243952 | 198870, 148248 |
| 53335 | 136282354 | 136283993 | 148300, 160323 |
| 53336 | 136313461 | 136314156 | 160323 |
| 53337 | 136324752 | 136326667 | 160325 |
| 53338 | 136343351 | 136344232 | 160326 |
| 53339 | 136371460 | 136372760 | |
| 53340 | 136374535 | 136375371 | |
| 53341 | 136385540 | 136387149 | 187616 |
| 53342 | 136523415 | 136524522 | 123454 |
| 53343 | 136579913 | 136580437 | 123453 |
| 53344 | 136602932 | 136603965 | 123453 |
| 53345 | 136604146 | 136605505 | 123453 |
| 53346 | 136633840 | 136634665 | 160293 |
| 53347 | 136654211 | 136655125 | 160293 |
| 53348 | 136683297 | 136684238 | 160293 |
| 53349 | 136719990 | 136722374 | 160293 |
| 53350 | 136744459 | 136745353 | 160293 |
| 53351 | 136745412 | 136748842 | 160293 |
| 53352 | 136749086 | 136751484 | 160293 |
| 53353 | 136751734 | 136755909 | 160293 |
| 53354 | 136757172 | 136759005 | 160293 |
| 53355 | 136779788 | 136780632 | 160293 |
| 53356 | 136838758 | 136840391 | 160293 |
| 53357 | 136843474 | 136845776 | 160293 |
| 53358 | 136855505 | 136860559 | 160293 |
| 53359 | 136875682 | 136876257 | |
| 53360 | 136890064 | 136891193 | 235106 |
| 53361 | 136893735 | 136894348 | 235106 |
| 53362 | 136914047 | 136914808 | 169925 |
| 53363 | 136920161 | 136922222 | 169925, 235138 |
| 53364 | 136930701 | 136933694 | 169925 |
| 53365 | 136987994 | 136988449 | |
| 53366 | 136995883 | 136996622 | |
| 53367 | 136999724 | 137002466 | 196363, 273249 |
| 53368 | 137028007 | 137030869 | 221676 |
| 53369 | 137040268 | 137041359 | |
| 53370 | 137102617 | 137103528 | |
| 53371 | 137110601 | 137114482 | |
| 53372 | 137117936 | 137118433 | |
| 53373 | 137150356 | 137151554 | |
| 53374 | 137155981 | 137157713 | |
| 53375 | 137159458 | 137160371 | |
| 53376 | 137174706 | 137175647 | |
| 53377 | 137214502 | 137218192 | 186350 |
| 53378 | 137218426 | 137222799 | 186350 |
| 53379 | 137224695 | 137226042 | 186350 |
| 53380 | 137226164 | 137228794 | 186350 |
| 53381 | 137252748 | 137254170 | 186350 |
| 53382 | 137256401 | 137257160 | 186350 |
| 53383 | 137261576 | 137273529 | 263897, 186350 |
| 53384 | 137274031 | 137275287 | 186350 |
| 53385 | 137310016 | 137310883 | 186350 |
| 53386 | 137326532 | 137327544 | 186350 |
| 53387 | 137334076 | 137335801 | |
| 53388 | 138370652 | 138372828 | 196422, 225361 |
| 53389 | 138380969 | 138384224 | |
| 53390 | 138391485 | 138393728 | 122140, 160345 |
| 53391 | 138797954 | 138800596 | 238058, 130559 |
| 53392 | 138851988 | 138853467 | 130560 |
| 53393 | 138884063 | 138885436 | |
| 53394 | 138885821 | 138886493 | |
| 53395 | 138899429 | 138901311 | 148411 |
| 53396 | 138985794 | 138987891 | 148411 |
| 53397 | 139000704 | 139002029 | 260193 |
| 53398 | 139008993 | 139012279 | 238227 |
| 53399 | 139136458 | 139138057 | 165661 |
| 53400 | 139257537 | 139258610 | 213221, 187796 |
| 53401 | 139290310 | 139290976 | 165684 |
| 53402 | 139293429 | 139295042 | |
| 53403 | 139303940 | 139306349 | 165688, 165689 |
| 53404 | 139312371 | 139313207 | 165688 |
| 53405 | 139332967 | 139335086 | 148396, 148384 |
| 53406 | 139335800 | 139337093 | 148396 |
| 53407 | 139376696 | 139380503 | 196366 |
| 53408 | 139414532 | 139414938 | 148400 |
| 53409 | 139439029 | 139442037 | 265181, 237886, 148400 |
| 53410 | 139444312 | 139446251 | 237886 |
| 53411 | 139452333 | 139453291 | |
| 53412 | 139461676 | 139462880 | |
| 53413 | 139470459 | 139471230 | |
| 53414 | 139513264 | 139514959 | |
| 53415 | 139519444 | 139522213 | |
| 53416 | 139560014 | 139560731 | 172889 |
| 53417 | 139580376 | 139582974 | 169692 |
| 53418 | 139584449 | 139585829 | |
| 53419 | 139587496 | 139591173 | 221693 |
| 53420 | 139591403 | 139593658 | |
| 53421 | 139621110 | 139623924 | 233016 |
| 53422 | 139685297 | 139686627 | 272896, 244187 |
| 53423 | 139694307 | 139695149 | 272679, 272896, 213213, |
| | | | 273066 |
| 53424 | 139701625 | 139703482 | 196642, 213213, 228544, |
| | | | 272679, 273066 |
| 53425 | 139735627 | 139736067 | 196642 |
| 53426 | 139742116 | 139744999 | 054148 |
| 53427 | 139752807 | 139754767 | 177943 |
| 53428 | 139758930 | 139761596 | 107223 |
| 53429 | 139776064 | 139778265 | 127191 |
| 53430 | 139779996 | 139782175 | 266507, 127191 |
| 53431 | 139794925 | 139798398 | 127191 |
| 53432 | 139828504 | 139829426 | |
| 53433 | 139831888 | 139833250 | 260190 |
| 53434 | 139833886 | 139835628 | 159069 |
| 53435 | 139837161 | 139840880 | 176919, 159069 |
| 53436 | 139841053 | 139843031 | 176919 |
| 53437 | 139843294 | 139845516 | 184925 |
| 53438 | 139886336 | 139887951 | 148362 |
| 53439 | 139916513 | 139918452 | 107331 |
| 53440 | 139949539 | 139950743 | |
| 53441 | 139979843 | 139982343 | 268996, 177239 |
| 53442 | 140008526 | 140009547 | 176978 |
| 53443 | 140023467 | 140025009 | |
| 53444 | 140082151 | 140084474 | 176101, 176248 |
| 53445 | 140091844 | 140093626 | 176058 |
| 53446 | 140094782 | 140095905 | 176058 |
| 53447 | 140099125 | 140100896 | 188566, 187713 |
| 53448 | 140115856 | 140116967 | 212864 |
| 53449 | 140117711 | 140118889 | |
| 53450 | 140134362 | 140137102 | 188229 |
| 53451 | 140148416 | 140149245 | |
| 53452 | 140149348 | 140150770 | 188986 |
| 53453 | 140316384 | 140319051 | 188747, 187609 |
| 53454 | 140349063 | 140350106 | 165802 |
| 53455 | 140352805 | 140354115 | 165802 |
| 53456 | 140376482 | 140377468 | 130653 |
| 53457 | 140444525 | 140445093 | 130653 |
| 53458 | 140445504 | 140447339 | 182154 |
| 53459 | 140472525 | 140474174 | 148399 |
| 53460 | 140483926 | 140485659 | 165724 |
| 53461 | 140499399 | 140499952 | |
| 53462 | 140500019 | 140501631 | 197070 |
| 53463 | 140505887 | 140508041 | 197070 |
| 53464 | 140511930 | 140514901 | 181090, 203993 |
| 53465 | 140564571 | 140565818 | 181090 |
| 53466 | 140583918 | 140585010 | 181090 |

The locations of ODSCs on Chromosome 10 are shown in Table 52.

TABLE 52
ODSC Locations on Chromosome 10

| SEQ ID NO. | Start | End | Overlapping Gene(s) |
|---|---|---|---|
| 53467 | 179204 | 182776 | 015171 |
| 53468 | 353978 | 355174 | 151240 |
| 53469 | 384000 | 384894 | 151240 |
| 53470 | 418654 | 420218 | 151240 |
| 53471 | 500831 | 501304 | 151240 |
| 53472 | 639283 | 640713 | 151240 |
| 53473 | 695044 | 697425 | 180525, 151240 |
| 53474 | 734126 | 736298 | 151240 |
| 53475 | 758001 | 758859 | |
| 53476 | 976120 | 978786 | 107929 |
| 53477 | 1016190 | 1016993 | |
| 53478 | 1033383 | 1035278 | 107937, 205740 |
| 53479 | 1093950 | 1096338 | 047056, 067064 |
| 53480 | 1101485 | 1103610 | 047056 |
| 53481 | 2121201 | 2122610 | 226694 |
| 53482 | 3087701 | 3088575 | |
| 53483 | 3109593 | 3111560 | 067057 |
| 53484 | 3132949 | 3134817 | 067057 |
| 53485 | 3136017 | 3139219 | 067057 |
| 53486 | 3144414 | 3146277 | 067057 |
| 53487 | 3148711 | 3150931 | 067057 |
| 53488 | 3153767 | 3154494 | 067057 |
| 53489 | 3213699 | 3215270 | 107959 |
| 53490 | 3239047 | 3240945 | |
| 53491 | 3407834 | 3408286 | |
| 53492 | 3506858 | 3508080 | |
| 53493 | 3510275 | 3512007 | |
| 53494 | 3710202 | 3711395 | |
| 53495 | 3762503 | 3763796 | |
| 53496 | 3768718 | 3771200 | |
| 53497 | 3780331 | 3784068 | |
| 53498 | 3790063 | 3791714 | |
| 53499 | 3814413 | 3830769 | 067082 |
| 53500 | 3844282 | 3856632 | |
| 53501 | 3889583 | 3896832 | |
| 53502 | 3909236 | 3912169 | |
| 53503 | 3928092 | 3930902 | |
| 53504 | 3931548 | 3933296 | |
| 53505 | 3944105 | 3945541 | |
| 53506 | 4283861 | 4286820 | 233117 |
| 53507 | 4501622 | 4502275 | |
| 53508 | 4583311 | 4584301 | |
| 53509 | 4700124 | 4702333 | 225269, 231298 |
| 53510 | 4702468 | 4707546 | 225269, 231298 |
| 53511 | 4718017 | 4721584 | 231298 |
| 53512 | 4763937 | 4765070 | |
| 53513 | 4767084 | 4769209 | |
| 53514 | 4893301 | 4894989 | |
| 53515 | 4944118 | 4945417 | 187134 |
| 53516 | 4957669 | 4958943 | 187134 |
| 53517 | 4981357 | 4992252 | 187134 |
| 53518 | 4998541 | 4999786 | 187134 |
| 53519 | 5005999 | 5008949 | 187134 |
| 53520 | 5023611 | 5028567 | 187134 |
| 53521 | 5028622 | 5029139 | |
| 53522 | 5051060 | 5052318 | 151632 |
| 53523 | 5057070 | 5063514 | 151632 |
| 53524 | 5074993 | 5076676 | |
| 53525 | 5082333 | 5085465 | 196139 |
| 53526 | 5085687 | 5086593 | 196139 |
| 53527 | 5111861 | 5115407 | 196139 |
| 53528 | 5134969 | 5140627 | 239142, 196139 |
| 53529 | 5145528 | 5146349 | 196139 |
| 53530 | 5146741 | 5148617 | 196139 |
| 53531 | 5149468 | 5152037 | 196139 |
| 53532 | 5188839 | 5191795 | |
| 53533 | 5224245 | 5227657 | 196326 |
| 53534 | 5229785 | 5230962 | 196326 |
| 53535 | 5237022 | 5241925 | 251909, 198610, 196326 |
| 53536 | 5258570 | 5259236 | 198610 |
| 53537 | 5275987 | 5276499 | 231039 |
| 53538 | 5285355 | 5285927 | 231039 |
| 53539 | 5323830 | 5327618 | 215267 |
| 53540 | 5333157 | 5334766 | |
| 53541 | 5454064 | 5455943 | 173848 |
| 53542 | 5487985 | 5492310 | 173848 |
| 53543 | 5560019 | 5560605 | 205488 |
| 53544 | 5570261 | 5570942 | |
| 53545 | 5571404 | 5572203 | |
| 53546 | 5588711 | 5591367 | |
| 53547 | 5616542 | 5617087 | |
| 53548 | 5650647 | 5651115 | 231483 |
| 53549 | 5651378 | 5653611 | 231483 |
| 53550 | 5668045 | 5668975 | |
| 53551 | 5673114 | 5675565 | 240577 |
| 53552 | 5686660 | 5688658 | 196372 |
| 53553 | 5693858 | 5694667 | 196372 |
| 53554 | 5701562 | 5702880 | 196372 |
| 53555 | 5707298 | 5709029 | 196372 |
| 53556 | 5710261 | 5710789 | |
| 53557 | 5725948 | 5728035 | 108021 |
| 53558 | 5734351 | 5735506 | 108021 |
| 53559 | 5793626 | 5795246 | 108021 |
| 53560 | 5854073 | 5856694 | 272764, 057608 |
| 53561 | 5924776 | 5925646 | 134461 |
| 53562 | 5930980 | 5932956 | 134452, 134461 |
| 53563 | 5972151 | 5972672 | 134452 |
| 53564 | 5992149 | 5993557 | 134470 |
| 53565 | 6007080 | 6007693 | 134470 |
| 53566 | 6009900 | 6011359 | 134470 |
| 53567 | 6013009 | 6014332 | 134470 |
| 53568 | 6017781 | 6020557 | 134470 |
| 53569 | 6050618 | 6051854 | |
| 53570 | 6092424 | 6093492 | 134460 |
| 53571 | 6095579 | 6096622 | 134460 |
| 53572 | 6130494 | 6132425 | 134453 |
| 53573 | 6156230 | 6157277 | 134453 |
| 53574 | 6186118 | 6187948 | 170525 |
| 53575 | 6205383 | 6206790 | 170525 |
| 53576 | 6233914 | 6234745 | 170525 |
| 53577 | 6244401 | 6246742 | 213994, 170525 |
| 53578 | 6316566 | 6318291 | |
| 53579 | 6320508 | 6322997 | 215244 |
| 53580 | 6339594 | 6342959 | 215244 |
| 53581 | 6407219 | 6407924 | |
| 53582 | 6443289 | 6444229 | |
| 53583 | 6622021 | 6623123 | 237943, 065675 |
| 53584 | 7737842 | 7740549 | |
| 53585 | 7741056 | 7743791 | |
| 53586 | 7743875 | 7750898 | 151655 |
| 53587 | 7757071 | 7757870 | 151655 |
| 53588 | 7829144 | 7831421 | 165629, 151657 |
| 53589 | 7859721 | 7861351 | 165632 |
| 53590 | 7950652 | 7951350 | 165632 |
| 53591 | 8092187 | 8093310 | 197308 |
| 53592 | 8223282 | 8224088 | |
| 53593 | 8225116 | 8225738 | |
| 53594 | 8407950 | 8409077 | |
| 53595 | 8953915 | 8954637 | 234752 |
| 53596 | 8955596 | 8956092 | 234752 |
| 53597 | 11564639 | 11565448 | 148429 |
| 53598 | 11575156 | 11575858 | 148429 |
| 53599 | 11580695 | 11582379 | 148429 |
| 53600 | 11593285 | 11595299 | 148429 |
| 53601 | 11636763 | 11637642 | 148429 |
| 53602 | 11644849 | 11645379 | 148429 |
| 53603 | 11651928 | 11654466 | 271360, 148429 |
| 53604 | 11681953 | 11683032 | |
| 53605 | 11739608 | 11740494 | |
| 53606 | 11740927 | 11741922 | |
| 53607 | 11757531 | 11759119 | |
| 53608 | 11783924 | 11786072 | 134463 |
| 53609 | 11798814 | 11800441 | 134463 |
| 53610 | 11824713 | 11825458 | |
| 53611 | 11835351 | 11837072 | |
| 53612 | 11863395 | 11868740 | 148426 |
| 53613 | 11872358 | 11875030 | 148426 |
| 53614 | 11883128 | 11883922 | 148426 |
| 53615 | 11888394 | 11888951 | 148426 |
| 53616 | 11906371 | 11907272 | 148426, 225778 |
| 53617 | 11916075 | 11919871 | 225778 |
| 53618 | 11921950 | 11925633 | 225778 |
| 53619 | 11938633 | 11942027 | |
| 53620 | 11942074 | 11944748 | |
| 53621 | 12000965 | 12001389 | 151461 |
| 53622 | 12083701 | 12086211 | 151461 |
| 53623 | 12087372 | 12088094 | |
| 53624 | 12094943 | 12096542 | |
| 53625 | 12110350 | 12114941 | 181192 |
| 53626 | 12170797 | 12172255 | 065665 |
| 53627 | 12198309 | 12199448 | 065665 |
| 53628 | 12234539 | 12236064 | 165609 |
| 53629 | 12236167 | 12239114 | 151465, 165609 |
| 53630 | 12307460 | 12308161 | |
| 53631 | 12341349 | 12342677 | |
| 53632 | 12364347 | 12365037 | |
| 53633 | 12370841 | 12371632 | |
| 53634 | 12374959 | 12376801 | |
| 53635 | 12390321 | 12393219 | 183049 |
| 53636 | 12446719 | 12448594 | 183049 |
| 53637 | 12487235 | 12488133 | 183049 |
| 53638 | 12498411 | 12499189 | 252118, 183049 |
| 53639 | 12512810 | 12513687 | 183049 |
| 53640 | 12572829 | 12573922 | 183049 |
| 53641 | 12647364 | 12648686 | 183049 |
| 53642 | 12661588 | 12662254 | 183049 |
| 53643 | 12670743 | 12672302 | 183049 |
| 53644 | 12672543 | 12674760 | 183049 |
| 53645 | 12691946 | 12692515 | 183049 |
| 53646 | 12700475 | 12700953 | 183049 |
| 53647 | 12741000 | 12742293 | 183049 |
| 53648 | 12742460 | 12743088 | 183049 |
| 53649 | 12744902 | 12750513 | 183049 |
| 53650 | 12759798 | 12763904 | 183049 |
| 53651 | 12886603 | 12887687 | |
| 53652 | 12908356 | 12908774 | |
| 53653 | 12914683 | 12915628 | |
| 53654 | 13035551 | 13036154 | 151468 |
| 53655 | 13141048 | 13144853 | 123240, 151468 |
| 53656 | 13149310 | 13150416 | 123240 |
| 53657 | 13190127 | 13190850 | |
| 53658 | 13202899 | 13203752 | 065328 |
| 53659 | 13215478 | 13216115 | 065328 |
| 53660 | 13312607 | 13314636 | |
| 53661 | 13324911 | 13325955 | 107537 |
| 53662 | 13340843 | 13342907 | 107537 |
| 53663 | 13343004 | 13344933 | 107537 |
| 53664 | 13388290 | 13391548 | 086475 |
| 53665 | 13491397 | 13493251 | 165626 |
| 53666 | 13557418 | 13557835 | 165626 |
| 53667 | 13568760 | 13572156 | 227175, 165626 |
| 53668 | 13628567 | 13629633 | 165630 |
| 53669 | 13720463 | 13720982 | 151474 |
| 53670 | 13721490 | 13722258 | 151474 |
| 53671 | 13743117 | 13744067 | 151474 |
| 53672 | 13745344 | 13745819 | 151474 |
| 53673 | 13748623 | 13750013 | 151474 |
| 53674 | 13759708 | 13760581 | 151474 |
| 53675 | 13807403 | 13808132 | 151474 |
| 53676 | 14026427 | 14027114 | 151474 |
| 53677 | 14090688 | 14092234 | 151474 |
| 53678 | 14116259 | 14116927 | 235410, 151474 |
| 53679 | 14574717 | 14575544 | 065809 |
| 53680 | 14577582 | 14578806 | 065809 |
| 53681 | 14585040 | 14585886 | 065809 |
| 53682 | 14592147 | 14593089 | 065809 |
| 53683 | 14597400 | 14599492 | 065809 |
| 53684 | 14604568 | 14607285 | 065809 |
| 53685 | 14611609 | 14612516 | 065809 |
| 53686 | 14612808 | 14614572 | 065809 |
| 53687 | 14619679 | 14621933 | 065809 |
| 53688 | 14628803 | 14631134 | 065809 |
| 53689 | 14633871 | 14634431 | 065809 |
| 53690 | 14643845 | 14644974 | 065809 |
| 53691 | 14645088 | 14648802 | 065809 |
| 53692 | 14653619 | 14654881 | 065809 |
| 53693 | 14738442 | 14739269 | 065809 |
| 53694 | 14778240 | 14779382 | 065809 |
| 53695 | 14790476 | 14792156 | 065809 |
| 53696 | 14824457 | 14825432 | |
| 53697 | 14879262 | 14881501 | 187522, 185267 |
| 53698 | 14919686 | 14921917 | 272853, 152455 |
| 53699 | 14995661 | 14996648 | 152457 |
| 53700 | 15000918 | 15002750 | 197889 |
| 53701 | 15138533 | 15140469 | 152464, 176236 |
| 53702 | 15150207 | 15151278 | 152464, 152465 |
| 53703 | 15178785 | 15180013 | 152464, 152465 |
| 53704 | 15193843 | 15194931 | 152465 |
| 53705 | 15209313 | 15210880 | 152465 |
| 53706 | 15218200 | 15218823 | |
| 53707 | 15222654 | 15223247 | |
| 53708 | 15241446 | 15245122 | |
| 53709 | 15376075 | 15376707 | 148468 |
| 53710 | 15377359 | 15378128 | 148468 |
| 53711 | 15412083 | 15413755 | 148468 |
| 53712 | 15859402 | 15860034 | 148481 |
| 53713 | 15878763 | 15879257 | 148481 |
| 53714 | 15900741 | 15902992 | 148481 |
| 53715 | 16422935 | 16423741 | |
| 53716 | 16478785 | 16479760 | 165983 |
| 53717 | 16482175 | 16483226 | 165983 |
| 53718 | 16858487 | 16860329 | 148484 |
| 53719 | 17103772 | 17106253 | 107611 |
| 53720 | 17157652 | 17158159 | 107611 |
| 53721 | 17242581 | 17244234 | 107614 |
| 53722 | 17270253 | 17271267 | 026025, 229124 |
| 53723 | 17658309 | 17659758 | 165996 |
| 53724 | 17684764 | 17688464 | 136738, 260589 |
| 53725 | 18550587 | 18551541 | 165995 |
| 53726 | 18601566 | 18602099 | 165995 |
| 53727 | 18614782 | 18615605 | 165995 |
| 53728 | 18899064 | 18901712 | 241058 |
| 53729 | 18904445 | 18905662 | 241058 |
| 53730 | 18909794 | 18910399 | 241058 |
| 53731 | 18916084 | 18916872 | 241058 |
| 53732 | 18925967 | 18929693 | 241058 |
| 53733 | 18938323 | 18941420 | 152487, 241058, 271786 |
| 53734 | 18946046 | 18949977 | 165997, 152487 |
| 53735 | 18956908 | 18957813 | 165997 |
| 53736 | 18970701 | 18972513 | |
| 53737 | 18988748 | 18989571 | |
| 53738 | 19674669 | 19675339 | 204740 |
| 53739 | 19926861 | 19928734 | 204740 |
| 53740 | 19945141 | 19946395 | 204740 |
| 53741 | 19947182 | 19948314 | 204740 |
| 53742 | 20126000 | 20126845 | 120594 |
| 53743 | 20161529 | 20162176 | 120594 |
| 53744 | 20211180 | 20212214 | 120594 |
| 53745 | 20217912 | 20219458 | 120594 |
| 53746 | 20220028 | 20222088 | 120594 |
| 53747 | 20292582 | 20293306 | 120594 |
| 53748 | 21274754 | 21275584 | 078114 |
| 53749 | 21277062 | 21277950 | 078114 |
| 53750 | 21350769 | 21351309 | 078114 |
| 53751 | 21381970 | 21382890 | 078114 |
| 53752 | 21386335 | 21387620 | 078114 |
| 53753 | 21461207 | 21463880 | 231920, 078114 |
| 53754 | 21473002 | 21473742 | |
| 53755 | 21483173 | 21484787 | |
| 53756 | 21581559 | 21582304 | |
| 53757 | 21625119 | 21626631 | |
| 53758 | 21652772 | 21654167 | 230109 |
| 53759 | 21687460 | 21688220 | |
| 53760 | 21783237 | 21786675 | 222071, 204682 |
| 53761 | 21794587 | 21795251 | |
| 53762 | 21798138 | 21801647 | |
| 53763 | 21805354 | 21806740 | 180592 |
| 53764 | 21807469 | 21808210 | 180592 |
| 53765 | 21808803 | 21809761 | 180592 |
| 53766 | 21813836 | 21817321 | 180592 |
| 53767 | 21821862 | 21824774 | 078403 |
| 53768 | 21873743 | 21874170 | 078403 |
| 53769 | 21876956 | 21877509 | 078403 |
| 53770 | 22215553 | 22216437 | 136770 |
| 53771 | 22291306 | 22293333 | 136770 |
| 53772 | 22517734 | 22518825 | |
| 53773 | 22604420 | 22606341 | 269897, 148444 |
| 53774 | 22608729 | 22611651 | 168283, 148444, 269897 |
| 53775 | 22613899 | 22615014 | 269897, 168283 |
| 53776 | 22629038 | 22630561 | 272366 |
| 53777 | 22716036 | 22717503 | 077327 |
| 53778 | 22722401 | 22724076 | 077327 |
| 53779 | 22725105 | 22727372 | 077327 |
| 53780 | 22755460 | 22756308 | |
| 53781 | 22757401 | 22758221 | |
| 53782 | 22843295 | 22844127 | 150867 |
| 53783 | 22912929 | 22913792 | 150867 |
| 53784 | 22944445 | 22945105 | 150867 |
| 53785 | 22969626 | 22973390 | 150867 |
| 53786 | 23000886 | 23003877 | 150867 |
| 53787 | 23007475 | 23008127 | |
| 53788 | 23068273 | 23068858 | |
| 53789 | 23354157 | 23355061 | |
| 53790 | 23384097 | 23385517 | 148450 |
| 53791 | 23388569 | 23389435 | 148450 |
| 53792 | 23626940 | 23627675 | 179133 |
| 53793 | 23727473 | 23729483 | 165312 |
| 53794 | 23794388 | 23795440 | |
| 53795 | 23800804 | 23802331 | |
| 53796 | 24496333 | 24498679 | 120549 |
| 53797 | 24581656 | 24582771 | 120549 |
| 53798 | 24716186 | 24718373 | 120549 |
| 53799 | 24721585 | 24722325 | 120549 |
| 53800 | 24751934 | 24756345 | 120549 |
| 53801 | 24771928 | 24773123 | 120549 |
| 53802 | 24931700 | 24932654 | 107863 |
| 53803 | 24995343 | 24997651 | 107863 |
| 53804 | 25011160 | 25014445 | 107863 |
| 53805 | 25051113 | 25052546 | |
| 53806 | 25190809 | 25191670 | 099256 |
| 53807 | 25240859 | 25242567 | 273107, 099256 |
| 53808 | 25246444 | 25247961 | |
| 53809 | 25251243 | 25253182 | |
| 53810 | 25282169 | 25282736 | 151023 |
| 53811 | 25305155 | 25306731 | 185875 |
| 53812 | 25347976 | 25354021 | |
| 53813 | 25367011 | 25370493 | |
| 53814 | 26623731 | 26624932 | |
| 53815 | 26654094 | 26655246 | |
| 53816 | 26726037 | 26730396 | 077420 |
| 53817 | 26756123 | 26758686 | 077420 |
| 53818 | 26771852 | 26773394 | 077420 |
| 53819 | 26773496 | 26778492 | 077420 |
| 53820 | 26780132 | 26781516 | 077420 |
| 53821 | 26844744 | 26845352 | 077420 |
| 53822 | 26860379 | 26861597 | |
| 53823 | 26900922 | 26901848 | |
| 53824 | 26910696 | 26912036 | |
| 53825 | 26985501 | 26987569 | 148459 |
| 53826 | 27022861 | 27023547 | 148459 |
| 53827 | 27069240 | 27071629 | 136754 |
| 53828 | 27147844 | 27150590 | 136754 |
| 53829 | 27388603 | 27390132 | 107890 |
| 53830 | 27442273 | 27444910 | 120539, 136758 |
| 53831 | 27528730 | 27532842 | 262412, 107897 |
| 53832 | 27541289 | 27542254 | 230445 |
| 53833 | 27554756 | 27555357 | 238021 |
| 53834 | 27608269 | 27609500 | |
| 53835 | 27672704 | 27673399 | |
| 53836 | 27792713 | 27794472 | 099246 |
| 53837 | 28030459 | 28032804 | 150051 |
| 53838 | 28400291 | 28401455 | 150054 |
| 53839 | 28408232 | 28410249 | 150054 |
| 53840 | 28459919 | 28461382 | 150054 |
| 53841 | 28504780 | 28505221 | 150054 |
| 53842 | 28591190 | 28592441 | 150054 |
| 53843 | 28654734 | 28655280 | |
| 53844 | 28820620 | 28823832 | 095787, 254635 |
| 53845 | 28858546 | 28859160 | 095787 |
| 53846 | 28876206 | 28877323 | 095787 |
| 53847 | 28881742 | 28882211 | 095787 |
| 53848 | 28922612 | 28923161 | |
| 53849 | 28965453 | 28968434 | 095739 |
| 53850 | 29023857 | 29024538 | |
| 53851 | 29043523 | 29044357 | |
| 53852 | 29058057 | 29058639 | |
| 53853 | 29086030 | 29087469 | 232624 |
| 53854 | 29096425 | 29097766 | 232624, 271220 |
| 53855 | 29127630 | 29129030 | |
| 53856 | 29162199 | 29162955 | 204365 |
| 53857 | 29227932 | 29229818 | |
| 53858 | 29271721 | 29272244 | |
| 53859 | 29273116 | 29273759 | |
| 53860 | 29280420 | 29281323 | |
| 53861 | 29393317 | 29394966 | |
| 53862 | 29418714 | 29419846 | |
| 53863 | 29429580 | 29431381 | |
| 53864 | 29431529 | 29432017 | |
| 53865 | 29438777 | 29439462 | |
| 53866 | 29452406 | 29453200 | |
| 53867 | 29528706 | 29529373 | |
| 53868 | 29555516 | 29556132 | |
| 53869 | 29606011 | 29609330 | 120563 |
| 53870 | 29611392 | 29612699 | |
| 53871 | 29612793 | 29613990 | |
| 53872 | 29615220 | 29617123 | |
| 53873 | 29617736 | 29618809 | |
| 53874 | 29663712 | 29664450 | |
| 53875 | 29697823 | 29699596 | 224597 |
| 53876 | 29907498 | 29908927 | 197321 |
| 53877 | 29912776 | 29913130 | 197321 |
| 53878 | 29937053 | 29938203 | 197321 |
| 53879 | 29947541 | 29949277 | 197321 |
| 53880 | 30002537 | 30003740 | 197321 |
| 53881 | 30023327 | 30026269 | 197321 |
| 53882 | 30050694 | 30051265 | |
| 53883 | 30091969 | 30096708 | |
| 53884 | 30099553 | 30101912 | |
| 53885 | 30119607 | 30120636 | |
| 53886 | 30129305 | 30130629 | |
| 53887 | 30130725 | 30131278 | |
| 53888 | 30142784 | 30143354 | |
| 53889 | 30215410 | 30216399 | |
| 53890 | 30235063 | 30239942 | |
| 53891 | 30243993 | 30247572 | |
| 53892 | 30288315 | 30291866 | |
| 53893 | 30449270 | 30449827 | |
| 53894 | 30471306 | 30472183 | |
| 53895 | 30474854 | 30476387 | |
| 53896 | 30636894 | 30639054 | 107951 |
| 53897 | 30721767 | 30724161 | 107968 |
| 53898 | 30781904 | 30784845 | |
| 53899 | 30792566 | 30793468 | |
| 53900 | 30799450 | 30800358 | |
| 53901 | 30826566 | 30827639 | |
| 53902 | 30830553 | 30832684 | |
| 53903 | 30842401 | 30843914 | 228800 |
| 53904 | 30846516 | 30847469 | |
| 53905 | 30872393 | 30873037 | |
| 53906 | 30876157 | 30877141 | |
| 53907 | 30894088 | 30895949 | |
| 53908 | 30900665 | 30901467 | 151033 |
| 53909 | 30905907 | 30906384 | 151033 |
| 53910 | 30927006 | 30927441 | |
| 53911 | 31072984 | 31074658 | |
| 53912 | 31269746 | 31270770 | 183621 |
| 53913 | 31272675 | 31275687 | 183621 |
| 53914 | 31279749 | 31281336 | 183621 |
| 53915 | 31287569 | 31290354 | 183621 |
| 53916 | 31319795 | 31321764 | 183621 |
| 53917 | 31422367 | 31424253 | |
| 53918 | 31531751 | 31532259 | 237036, 272381 |
| 53919 | 31606546 | 31610795 | 148516, 237036 |
| 53920 | 31632490 | 31633095 | 148516 |
| 53921 | 31641957 | 31643407 | 148516 |
| 53922 | 32043406 | 32045202 | 222412 |
| 53923 | 32046831 | 32049989 | |
| 53924 | 32153666 | 32154336 | 165322 |
| 53925 | 32170001 | 32171068 | 165322 |
| 53926 | 32177018 | 32177994 | 165322 |
| 53927 | 32216258 | 32220139 | 165322 |
| 53928 | 32228319 | 32234224 | |
| 53929 | 32236059 | 32237232 | |
| 53930 | 32343814 | 32345806 | 206660, 170759 |
| 53931 | 32400930 | 32401376 | |
| 53932 | 32583640 | 32584608 | 120616 |
| 53933 | 32634856 | 32637063 | 233825, 222309, 229327, |
| | | | 120616 |
| 53934 | 32665444 | 32668998 | 120616 |
| 53935 | 32734262 | 32736341 | 216937 |
| 53936 | 33076983 | 33077839 | 150076 |
| 53937 | 33243721 | 33248812 | 229656, 150093 |
| 53938 | 33260330 | 33260985 | 229656, 150093 |
| 53939 | 33268686 | 33275966 | 229656, 150093 |
| 53940 | 33292729 | 33296006 | 150093, 229656 |
| 53941 | 33296925 | 33300462 | 229656 |
| 53942 | 33417348 | 33420395 | |
| 53943 | 33422200 | 33427009 | |
| 53944 | 33431042 | 33433626 | |
| 53945 | 33433769 | 33435590 | |
| 53946 | 33443549 | 33447403 | |
| 53947 | 33482069 | 33483610 | 099250 |
| 53948 | 33484280 | 33486686 | 099250 |
| 53949 | 33526648 | 33527854 | 099250 |
| 53950 | 33551782 | 33555021 | 099250 |
| 53951 | 33561343 | 33563586 | 099250 |
| 53952 | 33564971 | 33566096 | 099250 |
| 53953 | 33579850 | 33580680 | 099250 |
| 53954 | 33613453 | 33614380 | 099250 |
| 53955 | 33622190 | 33626678 | 099250 |
| 53956 | 33765750 | 33766765 | |
| 53957 | 33796637 | 33797342 | |
| 53958 | 33798064 | 33798562 | |
| 53959 | 33860453 | 33860952 | |
| 53960 | 33901020 | 33902021 | |
| 53961 | 34087963 | 34089060 | |
| 53962 | 34090345 | 34091338 | |
| 53963 | 34165231 | 34166528 | |
| 53964 | 34188600 | 34189578 | |
| 53965 | 34409995 | 34411348 | 148498 |
| 53966 | 34413072 | 34414192 | 148498 |
| 53967 | 34473881 | 34474439 | 148498 |
| 53968 | 34569261 | 34569982 | 148498 |
| 53969 | 34579586 | 34580158 | 148498 |
| 53970 | 34652847 | 34654378 | 148498 |
| 53971 | 34826289 | 34827650 | 148498 |
| 53972 | 34840152 | 34840753 | 148498 |
| 53973 | 34986332 | 34989121 | 148498 |
| 53974 | 34998476 | 35001278 | 148498 |
| 53975 | 35002689 | 35003807 | 148498 |
| 53976 | 35006009 | 35006719 | 148498 |
| 53977 | 35016366 | 35017100 | 148498 |
| 53978 | 35020674 | 35023246 | 148498 |
| 53979 | 35025709 | 35027155 | 148498 |
| 53980 | 35035046 | 35035634 | 148498 |
| 53981 | 35046602 | 35048632 | 148498 |
| 53982 | 35102737 | 35105049 | 226386, 148498 |
| 53983 | 35283396 | 35284065 | |
| 53984 | 35324439 | 35324928 | 108094 |
| 53985 | 35378507 | 35380057 | 108094 |
| 53986 | 35414932 | 35417270 | 095794, 230534 |
| 53987 | 35483934 | 35485416 | 095794 |
| 53988 | 35497503 | 35497839 | 095794 |
| 53989 | 35500011 | 35500913 | 095794 |
| 53990 | 35505284 | 35505924 | |
| 53991 | 35508682 | 35510178 | 233200 |
| 53992 | 35535015 | 35535651 | |
| 53993 | 35556023 | 35557122 | 108100 |
| 53994 | 35619940 | 35622851 | 271335, 108100 |
| 53995 | 35622974 | 35626874 | 271335, 108100 |
| 53996 | 35653514 | 35654439 | 108100 |
| 53997 | 35708993 | 35712344 | 108100 |
| 53998 | 35712400 | 35715051 | 108100 |
| 53999 | 35736055 | 35738050 | 108100 |
| 54000 | 35740424 | 35741053 | 108100 |
| 54001 | 35742751 | 35743790 | 108100 |
| 54002 | 35751227 | 35751983 | 108100 |
| 54003 | 35784377 | 35784795 | 108100 |
| 54004 | 35831031 | 35831584 | 108100 |
| 54005 | 35930076 | 35931922 | 264780, 177283 |
| 54006 | 35974197 | 35974888 | |
| 54007 | 36053757 | 36054731 | |
| 54008 | 36056663 | 36057294 | |
| 54009 | 36398184 | 36399763 | |
| 54010 | 36403457 | 36404677 | |
| 54011 | 36970578 | 36971059 | |
| 54012 | 38145333 | 38147650 | 236514, 198105 |
| 54013 | 38149282 | 38150450 | |
| 54014 | 38264543 | 38266042 | 226447, 175395 |
| 54015 | 38299167 | 38300484 | 189180 |
| 54016 | 38382773 | 38384424 | 075407 |
| 54017 | 42384976 | 42385764 | |
| 54018 | 43047858 | 43048782 | 234420 |
| 54019 | 43125672 | 43126671 | 272373, 196693 |
| 54020 | 43132537 | 43134564 | 272373, 196693 |
| 54021 | 43277305 | 43279029 | 165733 |
| 54022 | 43447307 | 43448263 | |
| 54023 | 43521483 | 43522544 | |
| 54024 | 43549211 | 43549910 | |
| 54025 | 43555697 | 43556269 | |
| 54026 | 43600412 | 43601678 | 165731 |
| 54027 | 43632666 | 43635090 | 169826, 273008 |
| 54028 | 43757147 | 43757689 | 198915 |
| 54029 | 43800041 | 43800701 | |
| 54030 | 43804331 | 43804933 | |
| 54031 | 43878615 | 43879302 | |
| 54032 | 43890850 | 43893657 | 169813 |
| 54033 | 43894582 | 43895301 | 169813 |
| 54034 | 43901573 | 43905997 | 169813 |
| 54035 | 43915316 | 43917311 | 230555 |
| 54036 | 43931297 | 43932833 | 243660 |
| 54037 | 43950238 | 43951915 | 243660 |
| 54038 | 44069377 | 44070313 | 196793 |
| 54039 | 44101586 | 44102826 | 198298, 232004 |
| 54040 | 44143525 | 44144704 | 169740, 223910 |
| 54041 | 44184861 | 44186000 | |
| 54042 | 44357515 | 44358491 | 226808 |
| 54043 | 44358650 | 44360167 | 226808 |
| 54044 | 44503524 | 44504644 | |
| 54045 | 44690795 | 44691716 | |
| 54046 | 44732195 | 44732776 | |
| 54047 | 44886006 | 44886517 | |
| 54048 | 45360163 | 45360898 | 224812 |
| 54049 | 45406485 | 45407181 | 187783, 224812, 234504 |
| 54050 | 45422579 | 45423186 | 224812, 187783 |
| 54051 | 45430611 | 45431842 | 224812, 187783 |
| 54052 | 45435151 | 45435921 | 224812, 223462 |
| 54053 | 45453492 | 45455947 | 107551, 273363, 224812 |
| 54054 | 45461298 | 45462360 | 107551 |
| 54055 | 45469665 | 45471978 | 107551, 165507 |
| 54056 | 45472200 | 45478393 | 165507, 107551 |
| 54057 | 45485437 | 45486227 | 107551 |
| 54058 | 45490898 | 45492872 | 107551 |
| 54059 | 45494924 | 45497783 | 165512, 226937, 165511 |
| 54060 | 45914539 | 45915304 | 012779 |
| 54061 | 45946591 | 45947505 | 231964 |
| 54062 | 45965616 | 45966554 | 165406 |
| 54063 | 45966867 | 45967685 | 165406 |
| 54064 | 46089360 | 46090679 | 165406 |
| 54065 | 46122832 | 46123368 | 172671 |
| 54066 | 46167339 | 46169342 | 172671 |
| 54067 | 47008991 | 47009734 | |
| 54068 | 47074222 | 47074664 | 223477 |
| 54069 | 47092053 | 47093880 | 223477 |
| 54070 | 48354393 | 48355185 | 165388 |
| 54071 | 48398413 | 48398842 | |
| 54072 | 49514176 | 49515789 | 107643 |
| 54073 | 49517664 | 49518491 | 107643 |
| 54074 | 49571508 | 49574879 | 107643 |
| 54075 | 49732253 | 49732650 | 128805 |
| 54076 | 49863057 | 49864804 | 128805 |
| 54077 | 50236598 | 50237182 | 165633 |
| 54078 | 50257624 | 50258216 | 165633 |
| 54079 | 50279213 | 50279737 | 165633 |
| 54080 | 50284438 | 50285803 | 165633 |
| 54081 | 50287979 | 50290297 | 165633 |
| 54082 | 50315104 | 50315704 | 165633 |
| 54083 | 50322759 | 50323997 | 165633 |
| 54084 | 50384703 | 50386746 | 204161 |
| 54085 | 50397958 | 50398450 | |
| 54086 | 50570175 | 50571496 | |
| 54087 | 50746347 | 50748034 | 243251, 258838, 225830 |
| 54088 | 50967873 | 50968441 | 197444 |
| 54089 | 50968610 | 50970709 | 197444 |
| 54090 | 51044699 | 51046986 | 227345 |
| 54091 | 51561706 | 51562647 | 138294 |
| 54092 | 51563064 | 51567588 | 138293 |
| 54093 | 51571900 | 51574039 | 138293 |
| 54094 | 51574295 | 51576825 | 138293 |
| 54095 | 51580376 | 51581454 | 138293 |
| 54096 | 51592828 | 51593682 | 138297 |
| 54097 | 52009763 | 52010778 | |
| 54098 | 52145329 | 52145752 | 198964 |
| 54099 | 52151962 | 52156461 | 269700, 198964 |
| 54100 | 52156560 | 52157164 | 198964 |
| 54101 | 52177186 | 52178907 | 198964 |
| 54102 | 52257402 | 52258381 | 198964 |
| 54103 | 52270796 | 52272506 | 198964 |
| 54104 | 52274551 | 52275823 | 198964 |
| 54105 | 52298203 | 52299344 | 198964 |
| 54106 | 52309595 | 52310440 | 198964 |
| 54107 | 52323604 | 52324329 | 198964 |
| 54108 | 52325667 | 52326465 | 198964 |
| 54109 | 52331434 | 52332543 | 198964 |
| 54110 | 52342269 | 52343157 | 198964 |
| 54111 | 52352434 | 52354724 | 198964 |
| 54112 | 52364026 | 52365738 | 198964 |
| 54113 | 52366239 | 52366747 | 198964 |
| 54114 | 52381550 | 52386123 | 226200, 198964 |
| 54115 | 52499069 | 52500375 | 204147 |
| 54116 | 52567729 | 52569884 | 204147, 148584 |
| 54117 | 52611199 | 52612581 | 148584 |
| 54118 | 52614673 | 52616537 | 148584 |
| 54119 | 52641593 | 52646063 | 148584 |
| 54120 | 52653383 | 52653795 | |
| 54121 | 53458637 | 53460116 | 177613, 185532 |
| 54122 | 53972340 | 53973542 | 185532 |
| 54123 | 54527615 | 54528731 | 165471 |
| 54124 | 54530457 | 54532984 | 165471 |
| 54125 | 54533924 | 54535657 | |
| 54126 | 54643732 | 54644493 | |
| 54127 | 54766399 | 54767561 | |
| 54128 | 55120973 | 55121525 | |
| 54129 | 55163413 | 55163914 | |
| 54130 | 55646259 | 55647020 | 150275 |
| 54131 | 58120656 | 58121520 | 122952 |
| 54132 | 59388202 | 59389691 | |
| 54133 | 59453951 | 59457903 | |
| 54134 | 59463166 | 59464043 | |
| 54135 | 59536708 | 59537313 | |
| 54136 | 59624181 | 59625707 | |
| 54137 | 59719294 | 59719871 | |
| 54138 | 59752139 | 59752889 | |
| 54139 | 59782758 | 59783872 | |
| 54140 | 59788344 | 59789201 | |
| 54141 | 59874987 | 59875733 | |
| 54142 | 60025486 | 60028003 | 151151 |
| 54143 | 60028259 | 60030059 | 122873 |
| 54144 | 60040482 | 60041676 | 122873 |
| 54145 | 60082126 | 60082997 | |
| 54146 | 60094546 | 60095854 | 072401 |
| 54147 | 60144158 | 60145937 | 108064 |
| 54148 | 60227160 | 60229421 | |
| 54149 | 60412566 | 60413341 | 122870 |
| 54150 | 60421873 | 60422752 | 122870 |
| 54151 | 60514898 | 60516773 | 122870 |
| 54152 | 60572925 | 60574118 | 122870 |
| 54153 | 60577086 | 60578116 | 122870 |
| 54154 | 60580903 | 60582044 | 122870 |
| 54155 | 60583569 | 60584339 | 122870 |
| 54156 | 60615207 | 60617473 | |
| 54157 | 60654703 | 60655278 | |
| 54158 | 60656043 | 60656765 | |
| 54159 | 60702394 | 60703856 | |
| 54160 | 60727150 | 60727934 | |
| 54161 | 60753290 | 60755520 | |
| 54162 | 60758569 | 60761292 | 237949 |
| 54163 | 60765675 | 60768801 | |
| 54164 | 60796210 | 60797604 | |
| 54165 | 60821657 | 60822204 | |
| 54166 | 60833229 | 60833805 | |
| 54167 | 60935941 | 60937550 | 165443 |
| 54168 | 61075246 | 61075706 | 148541 |
| 54169 | 61081710 | 61082247 | 148541 |
| 54170 | 61082450 | 61083390 | 148541 |
| 54171 | 61096289 | 61097463 | 148541 |
| 54172 | 61121401 | 61123065 | 148541 |
| 54173 | 61370729 | 61371142 | 235140 |
| 54174 | 61631755 | 61634988 | 108091 |
| 54175 | 61664743 | 61668557 | 108091 |
| 54176 | 61709296 | 61709814 | |
| 54177 | 61724847 | 61725697 | |
| 54178 | 61731392 | 61732326 | |
| 54179 | 61848606 | 61849593 | 151150 |
| 54180 | 61856383 | 61857437 | 151150 |
| 54181 | 61907484 | 61908546 | 151150 |
| 54182 | 61931025 | 61931866 | 151150 |
| 54183 | 61946059 | 61946976 | 151150 |
| 54184 | 62042403 | 62043153 | 151150 |
| 54185 | 62144170 | 62145498 | 151150 |
| 54186 | 62162791 | 62163364 | 151150 |
| 54187 | 62182436 | 62183189 | 151150 |
| 54188 | 62185150 | 62187298 | 151150 |
| 54189 | 62331629 | 62333028 | 151150 |
| 54190 | 62387353 | 62388546 | 151150 |
| 54191 | 62537655 | 62538392 | 170312 |
| 54192 | 62698058 | 62698418 | 072422 |
| 54193 | 62702772 | 62705167 | 072422 |
| 54194 | 62715646 | 62716366 | 072422 |
| 54195 | 62723024 | 62725658 | 072422 |
| 54196 | 62760628 | 62761934 | 072422 |
| 54197 | 62822541 | 62823238 | |
| 54198 | 63594673 | 63598110 | |
| 54199 | 63656604 | 63657870 | |
| 54200 | 63659230 | 63665145 | 150347 |
| 54201 | 63702489 | 63703932 | 150347 |
| 54202 | 63723866 | 63725256 | 150347 |
| 54203 | 63777900 | 63778708 | 150347 |
| 54204 | 63781399 | 63782993 | 150347 |
| 54205 | 63797174 | 63799037 | 150347 |
| 54206 | 63805022 | 63806551 | 150347 |
| 54207 | 63807897 | 63811216 | 150347 |
| 54208 | 63836168 | 63837115 | 150347 |
| 54209 | 63990236 | 63992257 | 182010 |
| 54210 | 64192091 | 64194075 | 138311 |
| 54211 | 64319851 | 64320507 | 138311 |
| 54212 | 64322789 | 64325739 | 138311 |
| 54213 | 64563569 | 64565943 | 181915, 238280 |
| 54214 | 64576604 | 64578155 | 122877 |
| 54215 | 64873708 | 64875232 | |
| 54216 | 64875343 | 64876533 | |
| 54217 | 64892404 | 64894838 | 148572 |
| 54218 | 64896969 | 64897564 | 148572 |
| 54219 | 64995980 | 64996568 | 171988 |
| 54220 | 65025703 | 65029971 | 171988 |
| 54221 | 65190010 | 65190532 | 171988 |
| 54222 | 65224117 | 65226652 | 272767, 171988 |
| 54223 | 65280398 | 65282451 | 165476 |
| 54224 | 65388118 | 65391610 | |
| 54225 | 65459276 | 65461206 | 228566 |
| 54226 | 65461213 | 65462503 | 228566 |
| 54227 | 65466935 | 65468933 | 228566 |
| 54228 | 65469955 | 65473830 | 228566 |
| 54229 | 65478222 | 65480341 | 228566 |
| 54230 | 65497771 | 65498939 | 228566 |
| 54231 | 65814098 | 65814603 | |
| 54232 | 65835274 | 65835888 | |
| 54233 | 66129542 | 66130906 | |
| 54234 | 66792235 | 66793719 | |
| 54235 | 66870875 | 66871147 | |
| 54236 | 67260861 | 67261486 | |
| 54237 | 67278205 | 67279012 | |
| 54238 | 67709617 | 67710261 | 183230 |
| 54239 | 68043588 | 68044163 | 183230 |
| 54240 | 68080951 | 68082884 | 183230 |
| 54241 | 69048238 | 69048910 | 183230 |
| 54242 | 69523595 | 69525409 | |
| 54243 | 69559984 | 69560724 | 108176 |
| 54244 | 69563036 | 69563771 | 108176 |
| 54245 | 69579439 | 69580588 | 108176 |
| 54246 | 69587299 | 69588052 | 108176 |
| 54247 | 69608965 | 69610365 | 272892 |
| 54248 | 69643521 | 69645243 | 096717 |
| 54249 | 69649218 | 69649904 | 096717 |
| 54250 | 69719560 | 69720046 | 148634 |
| 54251 | 69833148 | 69835497 | 148634 |
| 54252 | 69845674 | 69848899 | |
| 54253 | 69856281 | 69857958 | |
| 54254 | 69872163 | 69873323 | 138347 |
| 54255 | 69894496 | 69897624 | 138347 |
| 54256 | 69910476 | 69916708 | 138347 |
| 54257 | 69923366 | 69924127 | 138347 |
| 54258 | 69990638 | 69991708 | 179774 |
| 54259 | 70061497 | 70062373 | 108187 |
| 54260 | 70063232 | 70064393 | 108187 |
| 54261 | 70065512 | 70066384 | 108187 |
| 54262 | 70087861 | 70088579 | 108187 |
| 54263 | 70090366 | 70093494 | 096746, 108187 |
| 54264 | 70165924 | 70167501 | 204130 |
| 54265 | 70286114 | 70287925 | 122912 |
| 54266 | 70301872 | 70303229 | |
| 54267 | 70319742 | 70320841 | 138336 |
| 54268 | 70359586 | 70360744 | 138336 |
| 54269 | 70407351 | 70408815 | 138336 |
| 54270 | 70480317 | 70482076 | 060339 |
| 54271 | 70659962 | 70661976 | 107625 |
| 54272 | 70714973 | 70717510 | 165732 |
| 54273 | 70748401 | 70749171 | 198954 |
| 54274 | 70882831 | 70884882 | 122958 |
| 54275 | 70920480 | 70921397 | 233163, 122958 |
| 54276 | 70938823 | 70941411 | 156502 |
| 54277 | 70962020 | 70962639 | 156502 |
| 54278 | 70975733 | 70978113 | 229261 |
| 54279 | 70979204 | 70980817 | 156510, 229261 |
| 54280 | 70981958 | 70982976 | 156510, 229261 |
| 54281 | 71074768 | 71075162 | 156515 |
| 54282 | 71168182 | 71169399 | 075073 |
| 54283 | 71389669 | 71390620 | 171224 |
| 54284 | 71443800 | 71447124 | 235645 |
| 54285 | 71455357 | 71456257 | |
| 54286 | 71528966 | 71529538 | |
| 54287 | 71650116 | 71650720 | 197467 |
| 54288 | 71812106 | 71814325 | 099284 |
| 54289 | 71845458 | 71846214 | 099284 |
| 54290 | 71850475 | 71851151 | 099284 |
| 54291 | 71868647 | 71869865 | 042286, 099284 |
| 54292 | 71890605 | 71893428 | 042286 |
| 54293 | 71904777 | 71907175 | 079332, 156521 |
| 54294 | 71914951 | 71915712 | 079332 |
| 54295 | 71929341 | 71931107 | 079332 |
| 54296 | 71991393 | 71994227 | 180817 |
| 54297 | 72082288 | 72084666 | 172731 |
| 54298 | 72090943 | 72092583 | 172731 |
| 54299 | 72112016 | 72112850 | 172731 |
| 54300 | 72141758 | 72142734 | 172731 |
| 54301 | 72147059 | 72147840 | |
| 54302 | 72163651 | 72165724 | 148730 |
| 54303 | 72190250 | 72190764 | |
| 54304 | 72475476 | 72476175 | 138316 |
| 54305 | 72575283 | 72576716 | 166224 |
| 54306 | 72595626 | 72596138 | 166224 |
| 54307 | 72613315 | 72614269 | 166224 |
| 54308 | 72645511 | 72646207 | 166228 |
| 54309 | 72647070 | 72648595 | 166228 |
| 54310 | 72976994 | 72977742 | 107731, 237512 |
| 54311 | 73006990 | 73007725 | 107731 |
| 54312 | 73025235 | 73028470 | 107731 |
| 54313 | 73052302 | 73053286 | 107731 |
| 54314 | 73067255 | 73069973 | |
| 54315 | 73078545 | 73080329 | 198246 |
| 54316 | 73093847 | 73094660 | 198246 |
| 54317 | 73096264 | 73098299 | 198246 |
| 54318 | 73098469 | 73101449 | 198246 |
| 54319 | 73102018 | 73104397 | 198246 |
| 54320 | 73106881 | 73108800 | 198246 |
| 54321 | 73136529 | 73137861 | |
| 54322 | 73233470 | 73234131 | 107736 |
| 54323 | 73356971 | 73358236 | 107736 |
| 54324 | 73372875 | 73373809 | 107736 |
| 54325 | 73394802 | 73395785 | 107736 |
| 54326 | 73397379 | 73399176 | 107736 |
| 54327 | 73524461 | 73528101 | 107738, 107736 |
| 54328 | 73559652 | 73560328 | 107736 |
| 54329 | 73605728 | 73606150 | 197746 |
| 54330 | 73609902 | 73611834 | 197746 |
| 54331 | 73619384 | 73621535 | |
| 54332 | 73630802 | 73631642 | |
| 54333 | 73647921 | 73650267 | |
| 54334 | 73734210 | 73737063 | 122863 |
| 54335 | 73828532 | 73829250 | 107742 |
| 54336 | 73855885 | 73859286 | 138303 |
| 54337 | 73867025 | 73867794 | 138303 |
| 54338 | 73975071 | 73977058 | 166295, 138303 |
| 54339 | 73989110 | 73989828 | 166295 |
| 54340 | 74007938 | 74008937 | |
| 54341 | 74014336 | 74015659 | |
| 54342 | 74019287 | 74021743 | |
| 54343 | 74023009 | 74023859 | |
| 54344 | 74031633 | 74038245 | 168209, 269926 |
| 54345 | 74057030 | 74058826 | |
| 54346 | 74066739 | 74071232 | |
| 54347 | 74074607 | 74077490 | |
| 54348 | 74077760 | 74083444 | |
| 54349 | 74087385 | 74097413 | 148719 |
| 54350 | 74112726 | 74115381 | 148719 |
| 54351 | 74262352 | 74263506 | 226163, 107745 |
| 54352 | 74283387 | 74284833 | 107745 |
| 54353 | 74384930 | 74386078 | 107745 |
| 54354 | 74397066 | 74397646 | |
| 54355 | 74398221 | 74399121 | |
| 54356 | 74447677 | 74448637 | |
| 54357 | 74451648 | 74452960 | 156026 |
| 54358 | 74500204 | 74500722 | 156026 |
| 54359 | 74712001 | 74715371 | 138308 |
| 54360 | 74848131 | 74848550 | 122884 |
| 54361 | 74854601 | 74857299 | 122884 |
| 54362 | 74873320 | 74873696 | 166321 |
| 54363 | 74877079 | 74878828 | 166321 |
| 54364 | 74891665 | 74892577 | 122882 |
| 54365 | 74895633 | 74897238 | 122882 |
| 54366 | 74926832 | 74928799 | 138286, 122882 |
| 54367 | 75006333 | 75007970 | 236756, 182180, 213551 |
| 54368 | 75011387 | 75013490 | 227540, 182180, 236756 |
| 54369 | 75118124 | 75118902 | 156042 |
| 54370 | 75172504 | 75174721 | 138279 |
| 54371 | 75254267 | 75256397 | 221817, 107758 |
| 54372 | 75335970 | 75336409 | 166348 |
| 54373 | 75337435 | 75338062 | 166348 |
| 54374 | 75346103 | 75346887 | 166348 |
| 54375 | 75385476 | 75386641 | 268584, 166348 |
| 54376 | 75408619 | 75409116 | 166317 |
| 54377 | 75489646 | 75490754 | 250959, 242338, 271816, |
| | | | 242288 |
| 54378 | 75502929 | 75505006 | 176986 |
| 54379 | 75510543 | 75511038 | 176986 |
| 54380 | 75531443 | 75533128 | 196968, 267964, 176986 |
| 54381 | 75541062 | 75542629 | 172586 |
| 54382 | 75544887 | 75546831 | 214655 |
| 54383 | 75570468 | 75571838 | 166507, 272916 |
| 54384 | 75576661 | 75577413 | 148660 |
| 54385 | 75579045 | 75580105 | 148660 |
| 54386 | 75599946 | 75600890 | 148660 |
| 54387 | 75613828 | 75614746 | 148660 |
| 54388 | 75633503 | 75635015 | 148660 |
| 54389 | 75647193 | 75647975 | |
| 54390 | 75757429 | 75759888 | 035403 |
| 54391 | 75777975 | 75778470 | 035403, 225761 |
| 54392 | 75786287 | 75787123 | 035403, 225761 |
| 54393 | 75815682 | 75817080 | 035403 |
| 54394 | 75909696 | 75912527 | 156110, 185009 |
| 54395 | 75935339 | 75937857 | 156110 |
| 54396 | 75949499 | 75950813 | 156110 |
| 54397 | 76308288 | 76309175 | 156110 |
| 54398 | 76584740 | 76587111 | 156650 |
| 54399 | 76600225 | 76601086 | 156650 |
| 54400 | 76676768 | 76678168 | 156650 |
| 54401 | 76683232 | 76685187 | 156650 |
| 54402 | 76735998 | 76736786 | 156650 |
| 54403 | 76870789 | 76872611 | 156671 |
| 54404 | 76897196 | 76898249 | 156671 |
| 54405 | 76951522 | 76952334 | |
| 54406 | 76960520 | 76963782 | |
| 54407 | 76969110 | 76972212 | 165637 |
| 54408 | 76994049 | 76996268 | 165644 |
| 54409 | 77054256 | 77055411 | 226051, 270087 |
| 54410 | 77156013 | 77157268 | 270087 |
| 54411 | 77159511 | 77164051 | 237149, 165655, 270087 |
| 54412 | 77168295 | 77169711 | 273248, 272692, 237149 |
| 54413 | 77190726 | 77192369 | 236842 |
| 54414 | 77197924 | 77198574 | |
| 54415 | 77352349 | 77353298 | |
| 54416 | 77365703 | 77366998 | 148655 |
| 54417 | 77423092 | 77425064 | 148655 |
| 54418 | 77440816 | 77441699 | 148655 |
| 54419 | 77492836 | 77493506 | 148655 |
| 54420 | 77515226 | 77515787 | 148655 |
| 54421 | 77530768 | 77534574 | 148655 |
| 54422 | 77547587 | 77549619 | 148655 |
| 54423 | 77718991 | 77719319 | 148655 |
| 54424 | 77783790 | 77787211 | 148655 |
| 54425 | 77792480 | 77794470 | 148655 |
| 54426 | 77795751 | 77797896 | 148655 |
| 54427 | 77810390 | 77811505 | 148655 |
| 54428 | 78044111 | 78045522 | 148655 |
| 54429 | 78083671 | 78084387 | 148655 |
| 54430 | 78087865 | 78088549 | 148655 |
| 54431 | 78095652 | 78096799 | 148655 |
| 54432 | 78153883 | 78154998 | 148655 |
| 54433 | 78191587 | 78193683 | 148655 |
| 54434 | 78251656 | 78252855 | 148655 |
| 54435 | 78670368 | 78671494 | 156113, 236467 |
| 54436 | 78832781 | 78833528 | 156113 |
| 54437 | 78838016 | 78839220 | 156113 |
| 54438 | 78921542 | 78925884 | 156113 |
| 54439 | 79006407 | 79007384 | 156113 |
| 54440 | 79009649 | 79010413 | 156113 |
| 54441 | 79047432 | 79048070 | 156113 |
| 54442 | 79055427 | 79057678 | 156113 |
| 54443 | 79093263 | 79094468 | 156113 |
| 54444 | 79109687 | 79111642 | 156113 |
| 54445 | 79132045 | 79132801 | 225652, 156113 |
| 54446 | 79136751 | 79137358 | 156113 |
| 54447 | 79165438 | 79166323 | 156113 |
| 54448 | 79190878 | 79191386 | 156113 |
| 54449 | 79239490 | 79240394 | 156113 |
| 54450 | 79245537 | 79246540 | 156113 |
| 54451 | 79257774 | 79259019 | 156113 |
| 54452 | 79260962 | 79262765 | 156113 |
| 54453 | 79290846 | 79291894 | 156113 |
| 54454 | 79292751 | 79294168 | 156113 |
| 54455 | 79314581 | 79316010 | 156113 |
| 54456 | 79323589 | 79325164 | 156113 |
| 54457 | 79339994 | 79341035 | 156113 |
| 54458 | 79361023 | 79361824 | 156113 |
| 54459 | 79397132 | 79398036 | 156113 |
| 54460 | 79497008 | 79498900 | |
| 54461 | 79641327 | 79641910 | 151208 |
| 54462 | 79658051 | 79659141 | 151208 |
| 54463 | 79661698 | 79662334 | 151208 |
| 54464 | 79667712 | 79671990 | 151208 |
| 54465 | 79680329 | 79681160 | 151208 |
| 54466 | 79685224 | 79687389 | 233871, 151208 |
| 54467 | 79748745 | 79749597 | 148606 |
| 54468 | 79788305 | 79790214 | 148606 |
| 54469 | 79793064 | 79794988 | 138326 |
| 54470 | 79806996 | 79807575 | 138326 |
| 54471 | 79925102 | 79925862 | |
| 54472 | 80014136 | 80019037 | 230417 |
| 54473 | 80062164 | 80063490 | 227136, 230417 |
| 54474 | 80198547 | 80199048 | 230417 |
| 54475 | 80205841 | 80207933 | 230417 |
| 54476 | 80216264 | 80220510 | 230417 |
| 54477 | 80272969 | 80273532 | 230417 |
| 54478 | 80288432 | 80289436 | 230417 |
| 54479 | 80298791 | 80299867 | 230417 |
| 54480 | 80311028 | 80311488 | 230417 |
| 54481 | 80383511 | 80385101 | 230417 |
| 54482 | 80388882 | 80390287 | 230417 |
| 54483 | 80502178 | 80503220 | |
| 54484 | 80515624 | 80516999 | |
| 54485 | 80534262 | 80535794 | |
| 54486 | 80538457 | 80539937 | |
| 54487 | 80575185 | 80576432 | |
| 54488 | 80731244 | 80735229 | 224596 |
| 54489 | 80736462 | 80738623 | 224596 |
| 54490 | 80757451 | 80758429 | 224596 |
| 54491 | 80818599 | 80820231 | 224596 |
| 54492 | 80826045 | 80831826 | 108175, 224596 |
| 54493 | 80833551 | 80834022 | 108175 |
| 54494 | 80853143 | 80854548 | 108175 |
| 54495 | 80856853 | 80857630 | 108175 |
| 54496 | 80871115 | 80873916 | 108175 |
| 54497 | 80886759 | 80887424 | 108175 |
| 54498 | 80908618 | 80911855 | 108175 |
| 54499 | 80913474 | 80913997 | 108175 |
| 54500 | 80915058 | 80919178 | 108175 |
| 54501 | 80922147 | 80923068 | 108175 |
| 54502 | 80943254 | 80944420 | 108175 |
| 54503 | 81005089 | 81008206 | 108175 |
| 54504 | 81033360 | 81034316 | 108175 |
| 54505 | 81057908 | 81060544 | 108175 |
| 54506 | 81079906 | 81081990 | |
| 54507 | 81090391 | 81092497 | |
| 54508 | 81094618 | 81096279 | |
| 54509 | 81106897 | 81109475 | 108179 |
| 54510 | 81138495 | 81143058 | 235426, 165424 |
| 54511 | 81158308 | 81159646 | 235426, 165424 |
| 54512 | 81168746 | 81169239 | 235426, 165424 |
| 54513 | 81183804 | 81184841 | 165424 |
| 54514 | 81203280 | 81206998 | 165424 |
| 54515 | 81232673 | 81233172 | |
| 54516 | 81388706 | 81389659 | 226676 |
| 54517 | 81664013 | 81665289 | 242600 |
| 54518 | 81686600 | 81687491 | 242600 |
| 54519 | 81741223 | 81742526 | 235924, 133661 |
| 54520 | 81837824 | 81839744 | 133678, 230091 |
| 54521 | 81955727 | 81957354 | 122359 |
| 54522 | 81962707 | 81968255 | 237523, 122359 |
| 54523 | 82029145 | 82029528 | |
| 54524 | 82031291 | 82034173 | 151224 |
| 54525 | 82040152 | 82044009 | 151224 |
| 54526 | 82047777 | 82051563 | 151224 |
| 54527 | 82052326 | 82052896 | |
| 54528 | 82081684 | 82085400 | |
| 54529 | 82093562 | 82094217 | 235858 |
| 54530 | 82166566 | 82169102 | 122378 |
| 54531 | 82175471 | 82176190 | 122378 |
| 54532 | 82212267 | 82215475 | 108219 |
| 54533 | 82215664 | 82216428 | 108219 |
| 54534 | 82236592 | 82238713 | 108219 |
| 54535 | 82254716 | 82255359 | 108219 |
| 54536 | 82258287 | 82260028 | 108219 |
| 54537 | 82261242 | 82261945 | 108219 |
| 54538 | 82263898 | 82266707 | 108219 |
| 54539 | 82295090 | 82296759 | 226659 |
| 54540 | 82311621 | 82312463 | 178217 |
| 54541 | 82660518 | 82660961 | |
| 54542 | 82977746 | 82978577 | |
| 54543 | 83359863 | 83360288 | |
| 54544 | 84013941 | 84014401 | 185737 |
| 54545 | 84026250 | 84026914 | 185737 |
| 54546 | 85898361 | 85900699 | 165678, 271933 |
| 54547 | 86087371 | 86089494 | 107771 |
| 54548 | 86175749 | 86176239 | 107771 |
| 54549 | 86527654 | 86528537 | |
| 54550 | 86584860 | 86585521 | |
| 54551 | 86620334 | 86621117 | |
| 54552 | 86905638 | 86906174 | |
| 54553 | 86952778 | 86954117 | 237267 |
| 54554 | 87039703 | 87040819 | |
| 54555 | 87044257 | 87045279 | |
| 54556 | 87316778 | 87317310 | |
| 54557 | 87362131 | 87363913 | 182771 |
| 54558 | 87400773 | 87405613 | 270002, 182771 |
| 54559 | 87501121 | 87502073 | 182771 |
| 54560 | 87647660 | 87648302 | 182771 |
| 54561 | 87752876 | 87754711 | 182771 |
| 54562 | 87755050 | 87756010 | 182771 |
| 54563 | 87922615 | 87924788 | 182771 |
| 54564 | 88120925 | 88121792 | 182771 |
| 54565 | 88125327 | 88127384 | 182771 |
| 54566 | 88159279 | 88163533 | |
| 54567 | 88168947 | 88171949 | |
| 54568 | 88195737 | 88203691 | 062650 |
| 54569 | 88280051 | 88283038 | 227896, 062650 |
| 54570 | 88294608 | 88296594 | |
| 54571 | 88352074 | 88353321 | |
| 54572 | 88353985 | 88354872 | |
| 54573 | 88361667 | 88362494 | |
| 54574 | 88470253 | 88472553 | 122367 |
| 54575 | 88514881 | 88518154 | 107779, 272631 |
| 54576 | 88534619 | 88535326 | 107779 |
| 54577 | 88691594 | 88692079 | 107779 |
| 54578 | 88730336 | 88732033 | 271880, 273413, 272734, |
| | | | 148671 |
| 54579 | 88852126 | 88855977 | 122376, 148672 |
| 54580 | 88902331 | 88902875 | 122376 |
| 54581 | 89260339 | 89260970 | |
| 54582 | 89263784 | 89266030 | 107789 |
| 54583 | 89338197 | 89339738 | |
| 54584 | 89345383 | 89346482 | |
| 54585 | 89350665 | 89351949 | 223761 |
| 54586 | 89358380 | 89358901 | 223761 |
| 54587 | 89378262 | 89379122 | 196566, 225913 |
| 54588 | 89394987 | 89396805 | 196566, 225913 |
| 54589 | 89405934 | 89407058 | 196566, 225913 |
| 54590 | 89415931 | 89417438 | 196566, 225913 |
| 54591 | 89418900 | 89421412 | 198682, 196566, 225913 |
| 54592 | 89445502 | 89446513 | 198682 |
| 54593 | 89460902 | 89463918 | 198682 |
| 54594 | 89576849 | 89579078 | 223820, 138138 |
| 54595 | 89601881 | 89603972 | 223820 |
| 54596 | 89621155 | 89624999 | 171862, 227268 |
| 54597 | 89712263 | 89712817 | 171862 |
| 54598 | 89715334 | 89716535 | 171862 |
| 54599 | 89719684 | 89720882 | 171862 |
| 54600 | 89732809 | 89734100 | |
| 54601 | 89735834 | 89736538 | |
| 54602 | 89787680 | 89789159 | |
| 54603 | 89821894 | 89824075 | |
| 54604 | 89824257 | 89824908 | |
| 54605 | 89839826 | 89840469 | |
| 54606 | 89855071 | 89856554 | |
| 54607 | 89869733 | 89871100 | |
| 54608 | 89878095 | 89879382 | |
| 54609 | 90002767 | 90004768 | |
| 54610 | 90013637 | 90014492 | |
| 54611 | 90145096 | 90147789 | 184719 |
| 54612 | 90188031 | 90188816 | 184719 |
| 54613 | 90234207 | 90234671 | 184719 |
| 54614 | 90248465 | 90249552 | 184719 |
| 54615 | 90342088 | 90343740 | 184719 |
| 54616 | 90378157 | 90378605 | 237205 |
| 54617 | 90552212 | 90553024 | |
| 54618 | 90587935 | 90588828 | 152766 |
| 54619 | 90599151 | 90601845 | 152766 |
| 54620 | 90608327 | 90608717 | 152766 |
| 54621 | 90639394 | 90641490 | 138134 |
| 54622 | 90694479 | 90695820 | 107796, 138134, 180139 |
| 54623 | 90709628 | 90712491 | 138134, 107796 |
| 54624 | 90734022 | 90735769 | 138134, 107796 |
| 54625 | 90738715 | 90739595 | 107796 |
| 54626 | 90748971 | 90752076 | 026103, 107796 |
| 54627 | 90923377 | 90923829 | |
| 54628 | 91001265 | 91002463 | 107798 |
| 54629 | 91010609 | 91012192 | 107798 |
| 54630 | 91029284 | 91030676 | 107798 |
| 54631 | 91037030 | 91039546 | 107798 |
| 54632 | 91060766 | 91063142 | 119922, 107798 |
| 54633 | 91086378 | 91088713 | 119917, 107798 |
| 54634 | 91091560 | 91093562 | 119917, 107798 |
| 54635 | 91105838 | 91106993 | 107798 |
| 54636 | 91110553 | 91112956 | 107798 |
| 54637 | 91133027 | 91135136 | 107798 |
| 54638 | 91151594 | 91152474 | 185745, 107798 |
| 54639 | 91173432 | 91175757 | 152778, 107798 |
| 54640 | 91184660 | 91186203 | |
| 54641 | 91191460 | 91192174 | 152779 |
| 54642 | 91228156 | 91228793 | 152779 |
| 54643 | 91234017 | 91242854 | 152779 |
| 54644 | 91275327 | 91276734 | 152779 |
| 54645 | 91295015 | 91296177 | 152779 |
| 54646 | 91312148 | 91315198 | 152779 |
| 54647 | 91350872 | 91352434 | 152782 |
| 54648 | 91361438 | 91362525 | 152782 |
| 54649 | 91392416 | 91393041 | 152782 |
| 54650 | 91403352 | 91406065 | 232936, 249962, 225836, |
| | | | 152782 |
| 54651 | 91415321 | 91415719 | |
| 54652 | 91459115 | 91459864 | 235100 |
| 54653 | 91460546 | 91461619 | 138182 |
| 54654 | 91681102 | 91681716 | 226159 |
| 54655 | 92630902 | 92632560 | 148688 |
| 54656 | 92647267 | 92648583 | 148688 |
| 54657 | 92669379 | 92670759 | |
| 54658 | 92671215 | 92682484 | 148677 |
| 54659 | 92719055 | 92721808 | |
| 54660 | 92765756 | 92766603 | 225519 |
| 54661 | 92802845 | 92803425 | |
| 54662 | 92921365 | 92924743 | |
| 54663 | 92958262 | 92958894 | |
| 54664 | 92979195 | 92981438 | 180628 |
| 54665 | 93057832 | 93058689 | |
| 54666 | 93108374 | 93112006 | |
| 54667 | 93113809 | 93114396 | |
| 54668 | 93168583 | 93171018 | 165338 |
| 54669 | 93271483 | 93272265 | 165338 |
| 54670 | 93285612 | 93287275 | |
| 54671 | 93315861 | 93317773 | |
| 54672 | 93349990 | 93351485 | |
| 54673 | 93363387 | 93364402 | |
| 54674 | 93367491 | 93369621 | |
| 54675 | 93388835 | 93394450 | 119938 |
| 54676 | 93401370 | 93404044 | |
| 54677 | 93411748 | 93412698 | |
| 54678 | 93449348 | 93451593 | |
| 54679 | 93460068 | 93461251 | |
| 54680 | 93465639 | 93467718 | |
| 54681 | 93469062 | 93469561 | |
| 54682 | 93547630 | 93548907 | 228701 |
| 54683 | 93556739 | 93559733 | 107854, 228701 |
| 54684 | 93651052 | 93651758 | |
| 54685 | 93660844 | 93662158 | |
| 54686 | 93667982 | 93669571 | 272817, 174721 |
| 54687 | 93682797 | 93684676 | 095564 |
| 54688 | 93786718 | 93787347 | 095564 |
| 54689 | 93860491 | 93862016 | 107864 |
| 54690 | 93863138 | 93863972 | 107864 |
| 54691 | 93881440 | 93883377 | 107864 |
| 54692 | 93883801 | 93885348 | 107864 |
| 54693 | 93949303 | 93949963 | 107864 |
| 54694 | 93982187 | 93983537 | 107864 |
| 54695 | 93997926 | 94001239 | 107864 |
| 54696 | 94049981 | 94052676 | 198060, 107864 |
| 54697 | 94087324 | 94090165 | 198060 |
| 54698 | 94332397 | 94335047 | 119912 |
| 54699 | 94350331 | 94353405 | 138160 |
| 54700 | 94404156 | 94404637 | 138160 |
| 54701 | 94448459 | 94452635 | 152804 |
| 54702 | 94454211 | 94458472 | 152804 |
| 54703 | 94458897 | 94460192 | |
| 54704 | 94607068 | 94609150 | 138190 |
| 54705 | 94651783 | 94652801 | 138190 |
| 54706 | 94731672 | 94732400 | 138190 |
| 54707 | 94791654 | 94793246 | 138190 |
| 54708 | 94831145 | 94832594 | |
| 54709 | 94837545 | 94838401 | 095596 |
| 54710 | 94865792 | 94866859 | 232805 |
| 54711 | 94941059 | 94941617 | |
| 54712 | 94942415 | 94943137 | |
| 54713 | 94958401 | 94959894 | |
| 54714 | 94980365 | 94980796 | |
| 54715 | 94985398 | 94986768 | |
| 54716 | 94995097 | 94996787 | |
| 54717 | 95146413 | 95147120 | 138119 |
| 54718 | 95225156 | 95226956 | 138119 |
| 54719 | 95230025 | 95231907 | 138119 |
| 54720 | 95241298 | 95242957 | 138119 |
| 54721 | 95325901 | 95326629 | 186188 |
| 54722 | 95338779 | 95339202 | 186188 |
| 54723 | 95348591 | 95350742 | 186188 |
| 54724 | 95357381 | 95364078 | 138207, 186188 |
| 54725 | 95412384 | 95413433 | 095464 |
| 54726 | 95431365 | 95433120 | 148690 |
| 54727 | 95461444 | 95463412 | 148690 |
| 54728 | 95532671 | 95533966 | 108231 |
| 54729 | 95534963 | 95535834 | 108231 |
| 54730 | 95652912 | 95654772 | 176273 |
| 54731 | 95676294 | 95677252 | 176273 |
| 54732 | 95751370 | 95752346 | |
| 54733 | 95753448 | 95754007 | 138193 |
| 54734 | 95766591 | 95767330 | 138193 |
| 54735 | 95824925 | 95825752 | 138193 |
| 54736 | 95835960 | 95836628 | 138193 |
| 54737 | 95882351 | 95884240 | 138193 |
| 54738 | 95892906 | 95893609 | 138193 |
| 54739 | 95972263 | 95974002 | 138193 |
| 54740 | 95992418 | 95993208 | 138193 |
| 54741 | 96075651 | 96076415 | 173145, 138193 |
| 54742 | 96121788 | 96123475 | 173145 |
| 54743 | 96161803 | 96164059 | 108239 |
| 54744 | 96304673 | 96306290 | 119969 |
| 54745 | 96430144 | 96448598 | 108242, 165841 |
| 54746 | 96492049 | 96493086 | 108242, 165841 |
| 54747 | 96493937 | 96495647 | 108242, 165841 |
| 54748 | 96507466 | 96508207 | 165841 |
| 54749 | 96661443 | 96663547 | |
| 54750 | 96695987 | 96697032 | |
| 54751 | 96750259 | 96753021 | |
| 54752 | 96794300 | 96796315 | |
| 54753 | 96825307 | 96826185 | 138115 |
| 54754 | 96828926 | 96829901 | 138115 |
| 54755 | 96876456 | 96877579 | |
| 54756 | 96892550 | 96893240 | |
| 54757 | 96942324 | 96943897 | 225533 |
| 54758 | 96989305 | 96991139 | 234026 |
| 54759 | 97002512 | 97003180 | 107438 |
| 54760 | 97019500 | 97021742 | 107438 |
| 54761 | 97025956 | 97027490 | 107438 |
| 54762 | 97032498 | 97034787 | 107438 |
| 54763 | 97037591 | 97042346 | 107438 |
| 54764 | 97046303 | 97047712 | 107438 |
| 54765 | 97047931 | 97051403 | 107438 |
| 54766 | 97054478 | 97055595 | |
| 54767 | 97068287 | 97069098 | |
| 54768 | 97089584 | 97091409 | 095637 |
| 54769 | 97109944 | 97110645 | 095637 |
| 54770 | 97147982 | 97148804 | 095637 |
| 54771 | 97195452 | 97196236 | 095637 |
| 54772 | 97200414 | 97201562 | 095637 |
| 54773 | 97204439 | 97206242 | 095637 |
| 54774 | 97216382 | 97218818 | 095637 |
| 54775 | 97233204 | 97236675 | 095637 |
| 54776 | 97238106 | 97241019 | 095637 |
| 54777 | 97241865 | 97243290 | 095637 |
| 54778 | 97244704 | 97245920 | 095637 |
| 54779 | 97261009 | 97263299 | 095637 |
| 54780 | 97272617 | 97275241 | 095637 |
| 54781 | 97276408 | 97278755 | 095637 |
| 54782 | 97288660 | 97289823 | 095637 |
| 54783 | 97312235 | 97313500 | 095637 |
| 54784 | 97320146 | 97321759 | 095637 |
| 54785 | 97415497 | 97416960 | 059573 |
| 54786 | 97452829 | 97454406 | 119977 |
| 54787 | 97666803 | 97668089 | 173088, 226688, 270099 |
| 54788 | 97802869 | 97804379 | 107443, 226688 |
| 54789 | 97848978 | 97850673 | 226688 |
| 54790 | 97884072 | 97885109 | |
| 54791 | 97889084 | 97891053 | 177853 |
| 54792 | 97995027 | 97995558 | 095585 |
| 54793 | 98003918 | 98004584 | 095585 |
| 54794 | 98029378 | 98032716 | 095585 |
| 54795 | 98035602 | 98036153 | |
| 54796 | 98046943 | 98048400 | |
| 54797 | 98273250 | 98273969 | 095587 |
| 54798 | 98345293 | 98348171 | 077147 |
| 54799 | 98397239 | 98398154 | 155629 |
| 54800 | 98423450 | 98424001 | 155629 |
| 54801 | 98432729 | 98434636 | 155629 |
| 54802 | 98435187 | 98437630 | 155629 |
| 54803 | 98454892 | 98455614 | 155629 |
| 54804 | 98478840 | 98481453 | 155629 |
| 54805 | 98590146 | 98594315 | 196233 |
| 54806 | 98595228 | 98596625 | 196233 |
| 54807 | 98600647 | 98601040 | 196233 |
| 54808 | 98602652 | 98603574 | 196233 |
| 54809 | 98610239 | 98611395 | 196233 |
| 54810 | 98623176 | 98624394 | 196233 |
| 54811 | 98757778 | 98758684 | 269891, 187122 |
| 54812 | 98783461 | 98784585 | 269891, 187122 |
| 54813 | 99051885 | 99052741 | 269891, 213390 |
| 54814 | 99057227 | 99058350 | |
| 54815 | 99067570 | 99068560 | |
| 54816 | 99078359 | 99081587 | 165879 |
| 54817 | 99087203 | 99089414 | |
| 54818 | 99091759 | 99095253 | 181274, 225850 |
| 54819 | 99185013 | 99188023 | 171314, 224474 |
| 54820 | 99204930 | 99206578 | 171307, 171311 |
| 54821 | 99226451 | 99226861 | 155229 |
| 54822 | 99257308 | 99259913 | 165886, 155229 |
| 54823 | 99331596 | 99333338 | 165887 |
| 54824 | 99333857 | 99335532 | 165887 |
| 54825 | 99343611 | 99345658 | 241935, 155252, 249967, |
| | | | 165887 |
| 54826 | 99354452 | 99358142 | 241935, 155252, 249967 |
| 54827 | 99392401 | 99393822 | 171160, 155252, 249967 |
| 54828 | 99400130 | 99402156 | 155252, 249967 |
| 54829 | 99407306 | 99407706 | 155252, 249967 |
| 54830 | 99423941 | 99424777 | 155252, 249967 |
| 54831 | 99446071 | 99448413 | 119986 |
| 54832 | 99473148 | 99474802 | 155254 |
| 54833 | 99478360 | 99478877 | |
| 54834 | 99496332 | 99498035 | 155256 |
| 54835 | 99621672 | 99622137 | 155265 |
| 54836 | 99628906 | 99629683 | 155265, 269783, 095713 |
| 54837 | 99641789 | 99643173 | 095713 |
| 54838 | 99893792 | 99896200 | 166024 |
| 54839 | 99930289 | 99930660 | 166024 |
| 54840 | 100022086 | 100023149 | 138131 |
| 54841 | 100027247 | 100030536 | 138131 |
| 54842 | 100032757 | 100033224 | |
| 54843 | 100034294 | 100034769 | |
| 54844 | 100082883 | 100083481 | |
| 54845 | 100178764 | 100181452 | 107521 |
| 54846 | 100193651 | 100195356 | 107521 |
| 54847 | 100205473 | 100207496 | 107521 |
| 54848 | 100209861 | 100210973 | |
| 54849 | 100219991 | 100221823 | 172987 |
| 54850 | 100226905 | 100228142 | 172987 |
| 54851 | 100229287 | 100229834 | 172987 |
| 54852 | 100443686 | 100444570 | 172987 |
| 54853 | 101051507 | 101052386 | |
| 54854 | 101062856 | 101066259 | |
| 54855 | 101098727 | 101099360 | 119946 |
| 54856 | 101132386 | 101132772 | 119946 |
| 54857 | 101135142 | 101135599 | 119946 |
| 54858 | 101155388 | 101156449 | |
| 54859 | 101171287 | 101172280 | 120053 |
| 54860 | 101180886 | 101181735 | 120053 |
| 54861 | 101188936 | 101191643 | 224934, 120053 |
| 54862 | 101378800 | 101382263 | 260475, 155287 |
| 54863 | 101407186 | 101408011 | |
| 54864 | 101418229 | 101421694 | 198018 |
| 54865 | 101426752 | 101427823 | 198018 |
| 54866 | 101432968 | 101433951 | 198018 |
| 54867 | 101490968 | 101493229 | 014919, 119929 |
| 54868 | 101513854 | 101515521 | 119929 |
| 54869 | 101534996 | 101546403 | 023839 |
| 54870 | 101546909 | 101549417 | 227351, 023839 |
| 54871 | 101609066 | 101609777 | 023839 |
| 54872 | 101686179 | 101688480 | 227695, 107554 |
| 54873 | 101689704 | 101692008 | 107554, 227695 |
| 54874 | 101704388 | 101705519 | 107554, 227695 |
| 54875 | 101741084 | 101742004 | 107554 |
| 54876 | 101765309 | 101767597 | 107554 |
| 54877 | 101768438 | 101770633 | 107554 |
| 54878 | 101803042 | 101804308 | 120054 |
| 54879 | 101860186 | 101862088 | |
| 54880 | 101906613 | 101909427 | |
| 54881 | 101923401 | 101924505 | 107566 |
| 54882 | 101938886 | 101940096 | 107566 |
| 54883 | 101944771 | 101946778 | 107566 |
| 54884 | 101988364 | 101989747 | 227492, 213341 |
| 54885 | 102026497 | 102028214 | 095485 |
| 54886 | 102044862 | 102047640 | 196072 |
| 54887 | 102050608 | 102052597 | 107593 |
| 54888 | 102100070 | 102102566 | 231188 |
| 54889 | 102104929 | 102107998 | 099194, 231188 |
| 54890 | 102118491 | 102120083 | 099194 |
| 54891 | 102124511 | 102125345 | 099194 |
| 54892 | 102130198 | 102131845 | |
| 54893 | 102132221 | 102135334 | 235823 |
| 54894 | 102152016 | 102152659 | |
| 54895 | 102241835 | 102242762 | 075290 |
| 54896 | 102278475 | 102279592 | 075826, 255339, 166136 |
| 54897 | 102288519 | 102290221 | 166135, 075826, 255339, |
| | | | 166136 |
| 54898 | 102295212 | 102296147 | 166135 |
| 54899 | 102490285 | 102491438 | |
| 54900 | 102635406 | 102635944 | |
| 54901 | 102672157 | 102673332 | 119906, 272572 |
| 54902 | 102689980 | 102690776 | 119906 |
| 54903 | 102728569 | 102730336 | 095539, 055950 |
| 54904 | 102730810 | 102731585 | 095539, 055950 |
| 54905 | 102746275 | 102748375 | 107815, 055950 |
| 54906 | 102756444 | 102759813 | 107816 |
| 54907 | 102791553 | 102792889 | 107819 |
| 54908 | 102802311 | 102803591 | |
| 54909 | 102820272 | 102821454 | 273162 |
| 54910 | 102825727 | 102828053 | 107821 |
| 54911 | 102872601 | 102873529 | 236311 |
| 54912 | 102880434 | 102881700 | 236311 |
| 54913 | 102973317 | 102974187 | |
| 54914 | 103051353 | 103052295 | |
| 54915 | 103112972 | 103114810 | 166167 |
| 54916 | 103123629 | 103124523 | 166167 |
| 54917 | 103126866 | 103128289 | 166167 |
| 54918 | 103346921 | 103348506 | 166169, 166171 |
| 54919 | 103453264 | 103455320 | 107829 |
| 54920 | 103576469 | 103579442 | 226009, 198408 |
| 54921 | 103657249 | 103657763 | 120029 |
| 54922 | 103755441 | 103755862 | 120029 |
| 54923 | 103769361 | 103770034 | 120029 |
| 54924 | 103814967 | 103816572 | 120029 |
| 54925 | 103824560 | 103826453 | 166189 |
| 54926 | 103873756 | 103875042 | 198728 |
| 54927 | 103879349 | 103880618 | 198728 |
| 54928 | 103891962 | 103894129 | 148840 |
| 54929 | 103910862 | 103913438 | 166197 |
| 54930 | 104004650 | 104006563 | 107862 |
| 54931 | 104043523 | 104044088 | 107862 |
| 54932 | 104070291 | 104071071 | 107862 |
| 54933 | 104153192 | 104156787 | 077150 |
| 54934 | 104158997 | 104159708 | 077150 |
| 54935 | 104180372 | 104182973 | 059915, 107872 |
| 54936 | 104191364 | 104192702 | 107874 |
| 54937 | 104194351 | 104195010 | |
| 54938 | 104195116 | 104196662 | 202569 |
| 54939 | 104209466 | 104211149 | 269609, 120055 |
| 54940 | 104212416 | 104213169 | 269609 |
| 54941 | 104220578 | 104221812 | 138111, 269609 |
| 54942 | 104260968 | 104262519 | 138107 |
| 54943 | 104263271 | 104264771 | 107882 |
| 54944 | 104356820 | 104357928 | 107882 |
| 54945 | 104386807 | 104388521 | 107882 |
| 54946 | 104401236 | 104407084 | 272933, 171206 |
| 54947 | 104473288 | 104474065 | 138175 |
| 54948 | 104474074 | 104474821 | 156398, 138175 |
| 54949 | 104502739 | 104505107 | 166272, 156398 |
| 54950 | 104529041 | 104531077 | 166272 |
| 54951 | 104534708 | 104537553 | 166272 |
| 54952 | 104539252 | 104541210 | 166272 |
| 54953 | 104541455 | 104544167 | 166272 |
| 54954 | 104561254 | 104561836 | 166272 |
| 54955 | 104573799 | 104574310 | 166272 |
| 54956 | 104609121 | 104610566 | |
| 54957 | 104613400 | 104616010 | 166275, 270316 |
| 54958 | 104629112 | 104630146 | 214435, 270316 |
| 54959 | 104675861 | 104679413 | 148842 |
| 54960 | 104748362 | 104749305 | 148842 |
| 54961 | 104780885 | 104782056 | 148842 |
| 54962 | 104880791 | 104881643 | 076685 |
| 54963 | 104905703 | 104906228 | 076685 |
| 54964 | 104951730 | 104953403 | 076685 |
| 54965 | 104956654 | 104957441 | |
| 54966 | 104990362 | 104991564 | |
| 54967 | 105001056 | 105001822 | |
| 54968 | 105109968 | 105110596 | 156374 |
| 54969 | 105127290 | 105128585 | 148835 |
| 54970 | 105155160 | 105157256 | 148843, 173915 |
| 54971 | 105173344 | 105173747 | 148843 |
| 54972 | 105408571 | 105409523 | 107957 |
| 54973 | 105411443 | 105413062 | 107957 |
| 54974 | 105419121 | 105422173 | 107957 |
| 54975 | 105553780 | 105554497 | 107957 |
| 54976 | 105571458 | 105572742 | 107957 |
| 54977 | 105615615 | 105616511 | |
| 54978 | 105667967 | 105668948 | 107960 |
| 54979 | 105677176 | 105678741 | 107960 |
| 54980 | 105726028 | 105727992 | 065613 |
| 54981 | 105852705 | 105854916 | |
| 54982 | 105858579 | 105859343 | |
| 54983 | 105869064 | 105870569 | |
| 54984 | 105878966 | 105882682 | 156384 |
| 54985 | 105957009 | 105958022 | 197748 |
| 54986 | 105991583 | 105992932 | 197748 |
| 54987 | 106012535 | 106017304 | 148834 |
| 54988 | 106028484 | 106029293 | 065621 |
| 54989 | 106096489 | 106100265 | 148841 |
| 54990 | 106108887 | 106111206 | |
| 54991 | 106334329 | 106335070 | |
| 54992 | 106567733 | 106570528 | 156395 |
| 54993 | 107734373 | 107735715 | |
| 54994 | 107746237 | 107747106 | |
| 54995 | 107752644 | 107754651 | |
| 54996 | 107770903 | 107771508 | |
| 54997 | 107870964 | 107871462 | |
| 54998 | 108657067 | 108658099 | 108018 |
| 54999 | 108690750 | 108691759 | 108018 |
| 55000 | 108730947 | 108731839 | 108018 |
| 55001 | 108813881 | 108814827 | 108018 |
| 55002 | 108815412 | 108816447 | 108018 |
| 55003 | 108854786 | 108855447 | 108018 |
| 55004 | 109731543 | 109732158 | 229981 |
| 55005 | 110118540 | 110119127 | |
| 55006 | 110221644 | 110222202 | |
| 55007 | 110319781 | 110322624 | 203434 |
| 55008 | 110337596 | 110338614 | |
| 55009 | 110341669 | 110344122 | |
| 55010 | 110357516 | 110359575 | |
| 55011 | 110389345 | 110390259 | |
| 55012 | 111674587 | 111675452 | 108039 |
| 55013 | 111681929 | 111683695 | 108039 |
| 55014 | 111766469 | 111768633 | 148700 |
| 55015 | 111896625 | 111897365 | |
| 55016 | 111900769 | 111901566 | |
| 55017 | 111967183 | 111967849 | 119950, 228417 |
| 55018 | 111968610 | 111971698 | 119950 |
| 55019 | 111981490 | 111982181 | 119950 |
| 55020 | 111985281 | 111987238 | 119950 |
| 55021 | 112028681 | 112029998 | 119950 |
| 55022 | 112063210 | 112066460 | 119953 |
| 55023 | 112118860 | 112119370 | |
| 55024 | 112123265 | 112124281 | |
| 55025 | 112154503 | 112160704 | |
| 55026 | 112169607 | 112172195 | |
| 55027 | 112172336 | 112173100 | |
| 55028 | 112215880 | 112216770 | 273143 |
| 55029 | 112219390 | 112221035 | 273143 |
| 55030 | 112255967 | 112259674 | 138166 |
| 55031 | 112260297 | 112261616 | 138166 |
| 55032 | 112263547 | 112264698 | 138166 |
| 55033 | 112326740 | 112328458 | 108055 |
| 55034 | 112431690 | 112432761 | 203867 |
| 55035 | 112484548 | 112485374 | 203867 |
| 55036 | 112495367 | 112495878 | 203867 |
| 55037 | 112533223 | 112535638 | 203867 |
| 55038 | 112542335 | 112542912 | 203867 |
| 55039 | 112552333 | 112553814 | 203867 |
| 55040 | 112605289 | 112606330 | |
| 55041 | 112611336 | 112613126 | |
| 55042 | 112616925 | 112618320 | |
| 55043 | 112630817 | 112633356 | 150593, 232470, 203497 |
| 55044 | 112645658 | 112646154 | 150593 |
| 55045 | 112677543 | 112680654 | 108061, 214413 |
| 55046 | 112722833 | 112723391 | 108061 |
| 55047 | 113235457 | 113235943 | |
| 55048 | 113316219 | 113317492 | |
| 55049 | 113731772 | 113732325 | |
| 55050 | 113884375 | 113885268 | |
| 55051 | 113936910 | 113937668 | 119927 |
| 55052 | 113972165 | 113976507 | 119927 |
| 55053 | 113982063 | 113991805 | |
| 55054 | 114001451 | 114005984 | |
| 55055 | 114020234 | 114022833 | |
| 55056 | 114024945 | 114026105 | |
| 55057 | 114031040 | 114032759 | |
| 55058 | 114033716 | 114037400 | |
| 55059 | 114037878 | 114041334 | |
| 55060 | 114055317 | 114058686 | 119913 |
| 55061 | 114060642 | 114065821 | 119913 |
| 55062 | 114092171 | 114092895 | 243316 |
| 55063 | 114107599 | 114108416 | 243316 |
| 55064 | 114111433 | 114112268 | 243316 |
| 55065 | 114119666 | 114123649 | |
| 55066 | 114123779 | 114125628 | |
| 55067 | 114133128 | 114137209 | 197142 |
| 55068 | 114138272 | 114141131 | 197142 |
| 55069 | 114142889 | 114143633 | 197142 |
| 55070 | 114154075 | 114154762 | 197142 |
| 55071 | 114205677 | 114207788 | 151532, 023041 |
| 55072 | 114398383 | 114399363 | 151532 |
| 55073 | 114475457 | 114477647 | 151532 |
| 55074 | 114487675 | 114489081 | 151532 |
| 55075 | 114499211 | 114500072 | 151532 |
| 55076 | 114501559 | 114502297 | 151532 |
| 55077 | 114510327 | 114511137 | 151532 |
| 55078 | 114563187 | 114565313 | 151532 |
| 55079 | 114571442 | 114572051 | 151532 |
| 55080 | 114574782 | 114576820 | 151532 |
| 55081 | 114580193 | 114582457 | |
| 55082 | 114599527 | 114600318 | |
| 55083 | 114602170 | 114603107 | |
| 55084 | 114612064 | 114612518 | |
| 55085 | 114635258 | 114636070 | |
| 55086 | 114655818 | 114656796 | 225292 |
| 55087 | 114657848 | 114660013 | 225292 |
| 55088 | 114673443 | 114676442 | |
| 55089 | 114708422 | 114713964 | 233547, 148737 |
| 55090 | 114715065 | 114716361 | 148737 |
| 55091 | 114766989 | 114767489 | 148737 |
| 55092 | 114778326 | 114778808 | 148737 |
| 55093 | 114780483 | 114782153 | 148737 |
| 55094 | 114790902 | 114791374 | 148737 |
| 55095 | 114795193 | 114796284 | 148737 |
| 55096 | 114801671 | 114802773 | 148737 |
| 55097 | 114832269 | 114835160 | 148737 |
| 55098 | 114845926 | 114848600 | 148737 |
| 55099 | 114852580 | 114854245 | 148737 |
| 55100 | 114858959 | 114859776 | 148737 |
| 55101 | 114866601 | 114867192 | 148737 |
| 55102 | 114871627 | 114874061 | 148737 |
| 55103 | 114882408 | 114883378 | 148737 |
| 55104 | 114885811 | 114886923 | 148737 |
| 55105 | 114890710 | 114893678 | 148737 |
| 55106 | 115085793 | 115086987 | |
| 55107 | 115144662 | 115146652 | |
| 55108 | 115193317 | 115196098 | |
| 55109 | 115206544 | 115206975 | |
| 55110 | 115215684 | 115216214 | |
| 55111 | 115239026 | 115241438 | |
| 55112 | 115264197 | 115264590 | |
| 55113 | 115283490 | 115284833 | |
| 55114 | 115297642 | 115302335 | |
| 55115 | 115309435 | 115316007 | 148702 |
| 55116 | 115316733 | 115318660 | 148702 |
| 55117 | 115320921 | 115322260 | 148702 |
| 55118 | 115322439 | 115323672 | 148702 |
| 55119 | 115326003 | 115327096 | 148702 |
| 55120 | 115345578 | 115346188 | 148702 |
| 55121 | 115390619 | 115391324 | 197893 |
| 55122 | 115404008 | 115404612 | 197893 |
| 55123 | 115438472 | 115441036 | 165806 |
| 55124 | 115506035 | 115506927 | |
| 55125 | 115612895 | 115615483 | 196865, 198924 |
| 55126 | 115693686 | 115694252 | |
| 55127 | 115697332 | 115698023 | |
| 55128 | 115700762 | 115701380 | |
| 55129 | 115704734 | 115705286 | |
| 55130 | 115717849 | 115720885 | |
| 55131 | 115823986 | 115824594 | |
| 55132 | 115910569 | 115911666 | 165813 |
| 55133 | 115932942 | 115934917 | 238742, 165813 |
| 55134 | 116149083 | 116149633 | 169129 |
| 55135 | 116230984 | 116231712 | 099204 |
| 55136 | 116246406 | 116248551 | 099204 |
| 55137 | 116264691 | 116265352 | 099204 |
| 55138 | 116277853 | 116280652 | 099204 |
| 55139 | 116285294 | 116287001 | 099204 |
| 55140 | 116308366 | 116308878 | 099204 |
| 55141 | 116316304 | 116317377 | 099204 |
| 55142 | 116334791 | 116338343 | 099204 |
| 55143 | 116363811 | 116368185 | 099204 |
| 55144 | 116371340 | 116373833 | 099204 |
| 55145 | 116383466 | 116385998 | 099204 |
| 55146 | 116388653 | 116395565 | 099204 |
| 55147 | 116406635 | 116411450 | 099204 |
| 55148 | 116411972 | 116413108 | 099204 |
| 55149 | 116414482 | 116415135 | 099204 |
| 55150 | 116424944 | 116425908 | 099204 |
| 55151 | 116438367 | 116439038 | 099204 |
| 55152 | 116440935 | 116446280 | 099204 |
| 55153 | 116478845 | 116479653 | |
| 55154 | 116483785 | 116484535 | |
| 55155 | 116513652 | 116515384 | |
| 55156 | 116526835 | 116528757 | 228484 |
| 55157 | 116580785 | 116583422 | 151553 |
| 55158 | 116585047 | 116586034 | 151553 |
| 55159 | 116615614 | 116617439 | 151553 |
| 55160 | 116697735 | 116699117 | 165832 |
| 55161 | 117325749 | 117326864 | 107518 |
| 55162 | 117705436 | 117709389 | 107518 |
| 55163 | 117764743 | 117765309 | |
| 55164 | 117797651 | 117798220 | |
| 55165 | 117804030 | 117804688 | |
| 55166 | 117805411 | 117805843 | |
| 55167 | 117814981 | 117817727 | 151892 |
| 55168 | 117839313 | 117841049 | 151892 |
| 55169 | 117842251 | 117842745 | 151892 |
| 55170 | 117943267 | 117943722 | 151892 |
| 55171 | 117953543 | 117954350 | 151892 |
| 55172 | 117966452 | 117967272 | 151892 |
| 55173 | 117970407 | 117971656 | 151892 |
| 55174 | 118030618 | 118034172 | 151892 |
| 55175 | 118230359 | 118231021 | 203837 |
| 55176 | 118501564 | 118502876 | 165868 |
| 55177 | 118530722 | 118531421 | |
| 55178 | 118608247 | 118609392 | 188316, 225302 |
| 55179 | 118626120 | 118627465 | 188316 |
| 55180 | 118640376 | 118641007 | 188316 |
| 55181 | 118647167 | 118649585 | 188316, 187164 |
| 55182 | 118762686 | 118765811 | 187164 |
| 55183 | 118766344 | 118767155 | 187164 |
| 55184 | 118927479 | 118928114 | 234474 |
| 55185 | 118934121 | 118934900 | |
| 55186 | 118946346 | 118946740 | |
| 55187 | 118975950 | 118977256 | |
| 55188 | 119123006 | 119126126 | 165650 |
| 55189 | 119131936 | 119132543 | 165650 |
| 55190 | 119133262 | 119135865 | 165650 |
| 55191 | 119151116 | 119152152 | |
| 55192 | 119163715 | 119165075 | |
| 55193 | 119173382 | 119174321 | |
| 55194 | 119198841 | 119199303 | 258114 |
| 55195 | 119255730 | 119256626 | 229847 |
| 55196 | 119805468 | 119807244 | 177640, 107560 |
| 55197 | 120070195 | 120070890 | 165669 |
| 55198 | 120081077 | 120082219 | 165669 |
| 55199 | 120101034 | 120102227 | 165669 |
| 55200 | 120183187 | 120183809 | |
| 55201 | 120489917 | 120490855 | 151893 |
| 55202 | 120501296 | 120502321 | 151893 |
| 55203 | 120503761 | 120504236 | 151893 |
| 55204 | 120513503 | 120514905 | 151893 |
| 55205 | 120522813 | 120523703 | |
| 55206 | 120538078 | 120538671 | |
| 55207 | 120553026 | 120553623 | |
| 55208 | 120630044 | 120630905 | |
| 55209 | 120699909 | 120700392 | |
| 55210 | 120718360 | 120719246 | |
| 55211 | 120766203 | 120766965 | |
| 55212 | 120784571 | 120785731 | |
| 55213 | 120788755 | 120789691 | 188613 |
| 55214 | 120789785 | 120790510 | 188613 |
| 55215 | 120838839 | 120841404 | 107581 |
| 55216 | 120863453 | 120864703 | 119979 |
| 55217 | 120872582 | 120874440 | 119979 |
| 55218 | 120879362 | 120881201 | 119979 |
| 55219 | 120895015 | 120895376 | 119979 |
| 55220 | 120923975 | 120926080 | 183605 |
| 55221 | 120937172 | 120938834 | 165672 |
| 55222 | 120965291 | 120968154 | 228485, 198873 |
| 55223 | 121000450 | 121001169 | 198873 |
| 55224 | 121002719 | 121003530 | 198873 |
| 55225 | 121011299 | 121014669 | 198873 |
| 55226 | 121030224 | 121031920 | 198873 |
| 55227 | 121075108 | 121077529 | 198873 |
| 55228 | 121100446 | 121101299 | 198873 |
| 55229 | 121121467 | 121122103 | 198873 |
| 55230 | 121209798 | 121211444 | 198873 |
| 55231 | 121354531 | 121357012 | 151923 |
| 55232 | 121410047 | 121412339 | 151929 |
| 55233 | 121417696 | 121418919 | 151929 |
| 55234 | 121425243 | 121425808 | 151929 |
| 55235 | 121484575 | 121486438 | 198825 |
| 55236 | 121502575 | 121503339 | 198825 |
| 55237 | 121631245 | 121633816 | 197771 |
| 55238 | 121651286 | 121653023 | 107651, 197771 |
| 55239 | 122610057 | 122611994 | 120008, 227165 |
| 55240 | 122823146 | 122823934 | |
| 55241 | 122851703 | 122852376 | |
| 55242 | 122954298 | 122955568 | |
| 55243 | 122976048 | 122977084 | |
| 55244 | 123062588 | 123063273 | |
| 55245 | 123084157 | 123085811 | |
| 55246 | 123177423 | 123178583 | |
| 55247 | 123290294 | 123290972 | 066468 |
| 55248 | 123295730 | 123296322 | 066468 |
| 55249 | 123301902 | 123302806 | 066468 |
| 55250 | 123367958 | 123368796 | |
| 55251 | 123670118 | 123671281 | 107669 |
| 55252 | 123674162 | 123674714 | 107669 |
| 55253 | 123685653 | 123688840 | 226864, 107669 |
| 55254 | 123695074 | 123695749 | 226864 |
| 55255 | 123733782 | 123735319 | 107672 |
| 55256 | 123749572 | 123750291 | 138162 |
| 55257 | 123754453 | 123755190 | 138162 |
| 55258 | 123772884 | 123773413 | 138162 |
| 55259 | 123778159 | 123779637 | 138162 |
| 55260 | 123803616 | 123804916 | 138162 |
| 55261 | 123822049 | 123823276 | 138162 |
| 55262 | 123871205 | 123873985 | 138162 |
| 55263 | 123886751 | 123887208 | 138162 |
| 55264 | 123899716 | 123902339 | 138162 |
| 55265 | 123903618 | 123904800 | 138162 |
| 55266 | 123905737 | 123907692 | 138162 |
| 55267 | 123907924 | 123908881 | 138162 |
| 55268 | 123922265 | 123922873 | 138162 |
| 55269 | 123935672 | 123936229 | 138162 |
| 55270 | 123941905 | 123944134 | 138162 |
| 55271 | 124060150 | 124061804 | 138152 |
| 55272 | 124066233 | 124068923 | 138152 |
| 55273 | 124069263 | 124071449 | 138152 |
| 55274 | 124103940 | 124104924 | |
| 55275 | 124132508 | 124135962 | 107679 |
| 55276 | 124220167 | 124222705 | 166033 |
| 55277 | 124227758 | 124229721 | 166033 |
| 55278 | 124263957 | 124264552 | 166033 |
| 55279 | 124620347 | 124622027 | 138161, 213185 |
| 55280 | 124712878 | 124714212 | 179988, 119965 |
| 55281 | 124716611 | 124717454 | 179988 |
| 55282 | 124739225 | 124740523 | 179988 |
| 55283 | 124743297 | 124743829 | 179988 |
| 55284 | 124767326 | 124769911 | 196177, 095574 |
| 55285 | 124779480 | 124780458 | 196177 |
| 55286 | 124798816 | 124799304 | 196177 |
| 55287 | 124888289 | 124890044 | |
| 55288 | 124898868 | 124899410 | |
| 55289 | 124913390 | 124914960 | 154473 |
| 55290 | 124930236 | 124930882 | |
| 55291 | 124961928 | 124964681 | |
| 55292 | 125137285 | 125138035 | 230131 |
| 55293 | 125733781 | 125734319 | |
| 55294 | 126042781 | 126044061 | |
| 55295 | 126077549 | 126079075 | |
| 55296 | 126106619 | 126107910 | 065154 |
| 55297 | 126113882 | 126114542 | |
| 55298 | 126123327 | 126124161 | |
| 55299 | 126149756 | 126150930 | 107902 |
| 55300 | 126158918 | 126160094 | 107902 |
| 55301 | 126163964 | 126165346 | 107902 |
| 55302 | 126285162 | 126286219 | 107902 |
| 55303 | 126320437 | 126321182 | 258539, 189319 |
| 55304 | 126396566 | 126397380 | 258539, 233334, 189319 |
| 55305 | 126424718 | 126425994 | 258539, 189319 |
| 55306 | 126428837 | 126430322 | 258539, 189319 |
| 55307 | 126431645 | 126433751 | 189319, 258539 |
| 55308 | 126479516 | 126481289 | 258539, 203791 |
| 55309 | 126489975 | 126491373 | 165660 |
| 55310 | 126551965 | 126554650 | |
| 55311 | 126591489 | 126592215 | |
| 55312 | 126594535 | 126595824 | |
| 55313 | 126596192 | 126596806 | |
| 55314 | 126597661 | 126598637 | |
| 55315 | 126604159 | 126607715 | 249456 |
| 55316 | 126609802 | 126610372 | 249456 |
| 55317 | 126612835 | 126613802 | 249456 |
| 55318 | 126641842 | 126642473 | 019995 |
| 55319 | 126676398 | 126676719 | 175029, 019995 |
| 55320 | 126772786 | 126773893 | 175029 |
| 55321 | 127407259 | 127409163 | 107938, 234134, 224023 |
| 55322 | 127511063 | 127513210 | 107949, 188690 |
| 55323 | 127583926 | 127585315 | 203780, 089876 |
| 55324 | 127600483 | 127600984 | 203780 |
| 55325 | 127661635 | 127662305 | 233409, 203780 |
| 55326 | 128423212 | 128423894 | |
| 55327 | 128593318 | 128595142 | 150760 |
| 55328 | 128606749 | 128607347 | 150760 |
| 55329 | 128770645 | 128771725 | 150760 |
| 55330 | 128949864 | 128951116 | 150760, 188916 |
| 55331 | 129025675 | 129027050 | 150760 |
| 55332 | 129098273 | 129098912 | 150760 |
| 55333 | 129664459 | 129665487 | |
| 55334 | 129684576 | 129685634 | 180745 |
| 55335 | 129687692 | 129688775 | 180745 |
| 55336 | 129690324 | 129691564 | 180745 |
| 55337 | 129704874 | 129705710 | 132334 |
| 55338 | 129835434 | 129835932 | 132334 |
| 55339 | 129923429 | 129925051 | 148773 |
| 55340 | 129948034 | 129948808 | |
| 55341 | 129954618 | 129956278 | |
| 55342 | 129970952 | 129973194 | |
| 55343 | 130008352 | 130009409 | |
| 55344 | 130725615 | 130726136 | |
| 55345 | 130894083 | 130896211 | |
| 55346 | 130900444 | 130902867 | |
| 55347 | 131184548 | 131185449 | |
| 55348 | 131264450 | 131267020 | 170430 |
| 55349 | 131268352 | 131269183 | 170430 |
| 55350 | 131298799 | 131299834 | 170430 |
| 55351 | 131329041 | 131331230 | 170430 |
| 55352 | 131358524 | 131359155 | 170430 |
| 55353 | 131439990 | 131441778 | 170430 |
| 55354 | 131507936 | 131509247 | 170430 |
| 55355 | 131553181 | 131553958 | 170430 |
| 55356 | 131555329 | 131556144 | 170430 |
| 55357 | 131651779 | 131652253 | 108001 |
| 55358 | 131908029 | 131909814 | 237489 |
| 55359 | 131933689 | 131937165 | 108010 |
| 55360 | 132098336 | 132099570 | |
| 55361 | 133274265 | 133277448 | |
| 55362 | 133462992 | 133463742 | |
| 55363 | 133646686 | 133647319 | |
| 55364 | 133792994 | 133796605 | 176171 |
| 55365 | 133809943 | 133810778 | |
| 55366 | 133810919 | 133811925 | |
| 55367 | 133836453 | 133837583 | |
| 55368 | 133878748 | 133879687 | |
| 55369 | 133996381 | 133997104 | 188385, 235010 |
| 55370 | 134133522 | 134134584 | 165752 |
| 55371 | 134144570 | 134146446 | 148814, 165752 |
| 55372 | 134208962 | 134212051 | 171813 |
| 55373 | 134330062 | 134331674 | 226900 |
| 55374 | 134331722 | 134333166 | |
| 55375 | 134349814 | 134352621 | 068383 |
| 55376 | 134403239 | 134405978 | 068383 |
| 55377 | 134489397 | 134490257 | 068383 |
| 55378 | 134778046 | 134778503 | 232903 |
| 55379 | 134974902 | 134976710 | 171798 |
| 55380 | 135074764 | 135075694 | |
| 55381 | 135091496 | 135092295 | |
| 55382 | 135121275 | 135122947 | 198546, 130640 |
| 55383 | 135131951 | 135132571 | 198546 |
| 55384 | 135160508 | 135162385 | 165828, 226699, 198546 |
| 55385 | 135163762 | 135165108 | 226699, 198546, 165828 |
| 55386 | 135166692 | 135167581 | |
| 55387 | 135170032 | 135171771 | 148803 |
| 55388 | 135172886 | 135174014 | |
| 55389 | 135185973 | 135187689 | 127884 |
| 55390 | 135191399 | 135193600 | 268882, 148832 |
| 55391 | 135207440 | 135208709 | 148824, 254536 |
| 55392 | 135325190 | 135326058 | 214279, 203772 |
| 55393 | 135333514 | 135334795 | 130649, 214279, 203772 |
| 55394 | 135340153 | 135342787 | 268925, 130649, 203772 |

The locations of ODSCs on Chromosome 11 are shown in Table 53.

TABLE 53
ODSC Locations on Chromosome 11

| SEQ ID NO. | Start | End | Overlapping Gene(s) |
|---|---|---|---|
| 55395 | 206069 | 211090 | 177963, 177951 |
| 55396 | 235591 | 238075 | 185627, 142082 |
| 55397 | 279756 | 280758 | 174885 |
| 55398 | 288256 | 289889 | 142102, 255026 |
| 55399 | 314950 | 315462 | 185885, 185201 |
| 55400 | 315755 | 316896 | |
| 55401 | 326671 | 327921 | 255328, 142089 |
| 55402 | 352704 | 354405 | |
| 55403 | 354776 | 356700 | |
| 55404 | 406150 | 407547 | 185187 |
| 55405 | 416552 | 417342 | 185187 |
| 55406 | 447959 | 449057 | 174915 |
| 55407 | 449832 | 451235 | 174915 |
| 55408 | 469243 | 470600 | 174915 |
| 55409 | 505767 | 507532 | 023191 |
| 55410 | 518436 | 519084 | |
| 55411 | 534474 | 535782 | 174775 |
| 55412 | 559642 | 562256 | 099849, 185522, 254815 |
| 55413 | 567781 | 570051 | 199038, 247095 |
| 55414 | 575563 | 577692 | 070047 |
| 55415 | 577991 | 578918 | 070047 |
| 55416 | 579954 | 580900 | 070047 |
| 55417 | 586339 | 586801 | 070047 |
| 55418 | 587261 | 588989 | 070047 |
| 55419 | 606180 | 606785 | 070047 |
| 55420 | 612037 | 613488 | 185507, 070047 |
| 55421 | 614411 | 616207 | 185507 |
| 55422 | 623875 | 625657 | 099834 |
| 55423 | 647571 | 648145 | 177030 |
| 55424 | 655059 | 655824 | 177030 |
| 55425 | 657593 | 658200 | 177030 |
| 55426 | 679439 | 680154 | 177030 |
| 55427 | 693944 | 696813 | 177042, 177106, 177030 |
| 55428 | 704169 | 708995 | 269915, 177030, 177042, |
| | | | 177106 |
| 55429 | 712449 | 713461 | 269915, 177106 |
| 55430 | 746337 | 748767 | 177156 |
| 55431 | 755346 | 755947 | 177156 |
| 55432 | 776243 | 779235 | 255284, 177225 |
| 55433 | 794616 | 798592 | 177542 |
| 55434 | 808466 | 811032 | 177600, 177595 |
| 55435 | 818537 | 821906 | 177666 |
| 55436 | 825210 | 825930 | 177666, 255108 |
| 55437 | 832145 | 835522 | 177697, 255108 |
| 55438 | 841762 | 845458 | 214063, 177700 |
| 55439 | 910001 | 912450 | 177830 |
| 55440 | 914008 | 915931 | 177830 |
| 55441 | 925091 | 926924 | 183020 |
| 55442 | 952257 | 952901 | 183020 |
| 55443 | 959305 | 960038 | 183020 |
| 55444 | 1073057 | 1074003 | |
| 55445 | 1144683 | 1146887 | |
| 55446 | 1146998 | 1148789 | |
| 55447 | 1283279 | 1284084 | 117983 |
| 55448 | 1318158 | 1320936 | 078902 |
| 55449 | 1323618 | 1327094 | 078902 |
| 55450 | 1328807 | 1331952 | 255153, 078902 |
| 55451 | 1579377 | 1581299 | 184545 |
| 55452 | 1591981 | 1594744 | 233930, 184545 |
| 55453 | 1594851 | 1596366 | 233930 |
| 55454 | 1673752 | 1674845 | |
| 55455 | 1688814 | 1689614 | 205866, 256528 |
| 55456 | 1695074 | 1698335 | |
| 55457 | 1699854 | 1700588 | |
| 55458 | 1711684 | 1712854 | |
| 55459 | 1714183 | 1715690 | |
| 55460 | 1780914 | 1782587 | 235027, 117984 |
| 55461 | 1782932 | 1786069 | 229512, 265587, 117984, |
| | | | 235027 |
| 55462 | 1851854 | 1852585 | 149043 |
| 55463 | 1853426 | 1854292 | 149043 |
| 55464 | 1920267 | 1920877 | |
| 55465 | 1968047 | 1970075 | 214026 |
| 55466 | 1973845 | 1975334 | 214026 |
| 55467 | 2177587 | 2178956 | 129965 |
| 55468 | 2179285 | 2180904 | 129965 |
| 55469 | 2187125 | 2188424 | 180176 |
| 55470 | 2346249 | 2347678 | |
| 55471 | 2390627 | 2391788 | 238184 |
| 55472 | 2398529 | 2400002 | 238184, 110651 |
| 55473 | 2401978 | 2403039 | 110651 |
| 55474 | 2419803 | 2423031 | 184281 |
| 55475 | 2720010 | 2722904 | 269821, 053918 |
| 55476 | 2800216 | 2800720 | 053918 |
| 55477 | 2881471 | 2882433 | 229414 |
| 55478 | 2922603 | 2925406 | 254827, 110628 |
| 55479 | 2949518 | 2952416 | 181649 |
| 55480 | 2961210 | 2963238 | |
| 55481 | 3011431 | 3014153 | 205531, 183562 |
| 55482 | 3062761 | 3065787 | 110619 |
| 55483 | 3070744 | 3071714 | 110619 |
| 55484 | 3077416 | 3079283 | 110619 |
| 55485 | 3399617 | 3400953 | 005801 |
| 55486 | 3663301 | 3664147 | 167311 |
| 55487 | 3695407 | 3696386 | 110713 |
| 55488 | 3802533 | 3803153 | 238304, 110713 |
| 55489 | 3817837 | 3819916 | 148985, 110713 |
| 55490 | 3829034 | 3830851 | 148985 |
| 55491 | 3861351 | 3863340 | 177105 |
| 55492 | 3875877 | 3878393 | 263421, 229368, 228661, |
| | | | 167323 |
| 55493 | 3886580 | 3888000 | 167323 |
| 55494 | 3888071 | 3889497 | 167323 |
| 55495 | 3900706 | 3902758 | 167323 |
| 55496 | 3925353 | 3927847 | 167323 |
| 55497 | 3934807 | 3935656 | 167323 |
| 55498 | 3936958 | 3937743 | 167323 |
| 55499 | 3950475 | 3951315 | 167323 |
| 55500 | 4065104 | 4065987 | 167323 |
| 55501 | 4114940 | 4117491 | 167325 |
| 55502 | 4156028 | 4156623 | 167325 |
| 55503 | 4414123 | 4415222 | 132109 |
| 55504 | 4501734 | 4503185 | |
| 55505 | 4567819 | 4568735 | |
| 55506 | 4628899 | 4629985 | 167333 |
| 55507 | 5617331 | 5618648 | 121236, 258588, 239920, |
| | | | 196565 |
| 55508 | 5641972 | 5642919 | 239920, 196565, 258659, |
| | | | 258588 |
| 55509 | 5643910 | 5644487 | 239920, 196565, 258659, |
| | | | 258588 |
| 55510 | 5645304 | 5646409 | 239920, 196565, 258659, |
| | | | 258588 |
| 55511 | 5705231 | 5707068 | 132256 |
| 55512 | 5710552 | 5712513 | 132274, 132256 |
| 55513 | 6250700 | 6251759 | 051009 |
| 55514 | 6255074 | 6256789 | 132259, 051009 |
| 55515 | 6341070 | 6341928 | 170955 |
| 55516 | 6386554 | 6387558 | |
| 55517 | 6409038 | 6410030 | |
| 55518 | 6410123 | 6412362 | 166311 |
| 55519 | 6439779 | 6440968 | 166313 |
| 55520 | 6460109 | 6465028 | 110169 |
| 55521 | 6494374 | 6496046 | 110171 |
| 55522 | 6501637 | 6503869 | 132286, 265264, 132254 |
| 55523 | 6623839 | 6626294 | 166333, 254400, 132275 |
| 55524 | 6627405 | 6628931 | 166337, 166333 |
| 55525 | 6632335 | 6634840 | 166340, 166337 |
| 55526 | 6659000 | 6659595 | 166341 |
| 55527 | 7040778 | 7042320 | 158077, 149050 |
| 55528 | 7272848 | 7274583 | 170743 |
| 55529 | 7278061 | 7278618 | 170743 |
| 55530 | 7281940 | 7283852 | 170743 |
| 55531 | 7470541 | 7471412 | 251364, 170743 |
| 55532 | 7532587 | 7536031 | 166387, 251364, 183801 |
| 55533 | 7545477 | 7547343 | 166387 |
| 55534 | 7551871 | 7552796 | 166387 |
| 55535 | 7572431 | 7573440 | 166387 |
| 55536 | 7592751 | 7594010 | 254864, 166387 |
| 55537 | 7594276 | 7595613 | 166387 |
| 55538 | 7597022 | 7597974 | 166387 |
| 55539 | 7613261 | 7615368 | 166387 |
| 55540 | 7621316 | 7622011 | 166387 |
| 55541 | 7626644 | 7627250 | 166387 |
| 55542 | 7633283 | 7635487 | 166387 |
| 55543 | 7652239 | 7652875 | 166387 |
| 55544 | 7669279 | 7670177 | 166387 |
| 55545 | 7693806 | 7695512 | 166394 |
| 55546 | 7715782 | 7716473 | 183378 |
| 55547 | 8008021 | 8009286 | 175390 |
| 55548 | 8089765 | 8090970 | 248332, 166402 |
| 55549 | 8350300 | 8350862 | |
| 55550 | 8692577 | 8694256 | 166436 |
| 55551 | 8696353 | 8697162 | |
| 55552 | 8702983 | 8706781 | 166441, 200983 |
| 55553 | 8709437 | 8711638 | 166441 |
| 55554 | 8730475 | 8731439 | 166441, 166444 |
| 55555 | 8738436 | 8743521 | 166444 |
| 55556 | 8753178 | 8755831 | 166444 |
| 55557 | 8778376 | 8779412 | 166444 |
| 55558 | 8786524 | 8791685 | 255159, 166444 |
| 55559 | 8815576 | 8816898 | 166444, 255159 |
| 55560 | 8932548 | 8933462 | 166452 |
| 55561 | 8984840 | 8987215 | 254860, 175348 |
| 55562 | 9159275 | 9160933 | 184014, 175356 |
| 55563 | 9285264 | 9287598 | 184014 |
| 55564 | 9335076 | 9337135 | 166471 |
| 55565 | 9384790 | 9386933 | |
| 55566 | 9405799 | 9407424 | 205339 |
| 55567 | 9480968 | 9484164 | 166478, 268403 |
| 55568 | 9585972 | 9591959 | |
| 55569 | 9594078 | 9598629 | 166483 |
| 55570 | 9616042 | 9616621 | |
| 55571 | 9632305 | 9633179 | |
| 55572 | 9633272 | 9636873 | |
| 55573 | 9684860 | 9686467 | 133789 |
| 55574 | 9761673 | 9762266 | 133789 |
| 55575 | 9779030 | 9782299 | 246273, 245522 |
| 55576 | 9927914 | 9928621 | 255476, 133812 |
| 55577 | 9965253 | 9965739 | 133812 |
| 55578 | 10065487 | 10066869 | 133812 |
| 55579 | 10139535 | 10140185 | 133812 |
| 55580 | 10303507 | 10304226 | 133812 |
| 55581 | 10306654 | 10307480 | 133812 |
| 55582 | 10314417 | 10316702 | 133812 |
| 55583 | 10323555 | 10332048 | 254554, 133805, 148926 |
| 55584 | 10344025 | 10345639 | 133805 |
| 55585 | 10347456 | 10349231 | 133805 |
| 55586 | 10369834 | 10370937 | 133805 |
| 55587 | 10372643 | 10374534 | 133805 |
| 55588 | 10387688 | 10389070 | 133805 |
| 55589 | 10415696 | 10416751 | 133805 |
| 55590 | 10427481 | 10430175 | 133805 |
| 55591 | 10471591 | 10473146 | 133805 |
| 55592 | 10509979 | 10513253 | 133805 |
| 55593 | 10561589 | 10563205 | 177112, 110315 |
| 55594 | 10571807 | 10572749 | 177112 |
| 55595 | 10678905 | 10679486 | 072952 |
| 55596 | 10680698 | 10681810 | 072952 |
| 55597 | 10756731 | 10758587 | |
| 55598 | 10771790 | 10774035 | 198730 |
| 55599 | 10792838 | 10793461 | 198730 |
| 55600 | 10828739 | 10831759 | 246308, 110321 |
| 55601 | 10878176 | 10880890 | 247271, 236287 |
| 55602 | 10907005 | 10907869 | 247271, 250041 |
| 55603 | 10914819 | 10916690 | 247271, 250041 |
| 55604 | 10946587 | 10950414 | |
| 55605 | 10951783 | 10953662 | |
| 55606 | 11169516 | 11170370 | 254401 |
| 55607 | 11334171 | 11336716 | 110328 |
| 55608 | 11458022 | 11458853 | 110328 |
| 55609 | 11462604 | 11463855 | 110328 |
| 55610 | 11470779 | 11474647 | 110328 |
| 55611 | 11601174 | 11602760 | 110328 |
| 55612 | 11641422 | 11644214 | 110328 |
| 55613 | 11834168 | 11836722 | |
| 55614 | 11862278 | 11865997 | 170242 |
| 55615 | 11967486 | 11971446 | 170242 |
| 55616 | 11994444 | 11995439 | 050165 |
| 55617 | 11997725 | 11999781 | 050165 |
| 55618 | 12013225 | 12014817 | 050165 |
| 55619 | 12051429 | 12052252 | |
| 55620 | 12063713 | 12067118 | 254486 |
| 55621 | 12068128 | 12073220 | 254486 |
| 55622 | 12075275 | 12075793 | 254486 |
| 55623 | 12086674 | 12090344 | 254991 |
| 55624 | 12092330 | 12093762 | 254991 |
| 55625 | 12101004 | 12102591 | |
| 55626 | 12106591 | 12110300 | 255400 |
| 55627 | 12110503 | 12114178 | 255400 |
| 55628 | 12127172 | 12127722 | 133816 |
| 55629 | 12131180 | 12133402 | 133816 |
| 55630 | 12187674 | 12189824 | 133816 |
| 55631 | 12203973 | 12204690 | 133816 |
| 55632 | 12274746 | 12276153 | 133816 |
| 55633 | 12308324 | 12310518 | 133808 |
| 55634 | 12398396 | 12400477 | 197702 |
| 55635 | 12694823 | 12698004 | 187079 |
| 55636 | 12713107 | 12717783 | 187079 |
| 55637 | 12727374 | 12727976 | 187079 |
| 55638 | 12790942 | 12792471 | 187079 |
| 55639 | 12823899 | 12824995 | 187079 |
| 55640 | 12873312 | 12876838 | 187079 |
| 55641 | 12889784 | 12890382 | 187079 |
| 55642 | 12893331 | 12893953 | 187079 |
| 55643 | 13029413 | 13030082 | 254670 |
| 55644 | 13161530 | 13163031 | |
| 55645 | 13204525 | 13205477 | |
| 55646 | 13229209 | 13230233 | |
| 55647 | 13298266 | 13300718 | 133794 |
| 55648 | 13336480 | 13337604 | 133794 |
| 55649 | 13350535 | 13351754 | 133794 |
| 55650 | 13354196 | 13357464 | 133794 |
| 55651 | 13358917 | 13361788 | 133794 |
| 55652 | 13472573 | 13473195 | 148925 |
| 55653 | 13482892 | 13485936 | 148925 |
| 55654 | 13497300 | 13498306 | |
| 55655 | 13986662 | 13987587 | 152268 |
| 55656 | 14037147 | 14037703 | 152268 |
| 55657 | 14051306 | 14051893 | 152268 |
| 55658 | 14135610 | 14136369 | 152268 |
| 55659 | 14211372 | 14212023 | 152268 |
| 55660 | 14279013 | 14280679 | 152268 |
| 55661 | 14309505 | 14310767 | 133818 |
| 55662 | 14357194 | 14359729 | 133818 |
| 55663 | 14378557 | 14381339 | 133818 |
| 55664 | 14394834 | 14397557 | |
| 55665 | 14397846 | 14399174 | |
| 55666 | 14401905 | 14403776 | |
| 55667 | 14409049 | 14411281 | |
| 55668 | 14432994 | 14434212 | |
| 55669 | 14442474 | 14446683 | |
| 55670 | 14464838 | 14471335 | 129083 |
| 55671 | 14520503 | 14522435 | 129083, 129084, 256206 |
| 55672 | 14540115 | 14542630 | 256206, 129084 |
| 55673 | 14562636 | 14563333 | 129084 |
| 55674 | 14604440 | 14606875 | 129084 |
| 55675 | 14639137 | 14640168 | 129084 |
| 55676 | 14640969 | 14642327 | 129084 |
| 55677 | 14664131 | 14666914 | 152270, 129084 |
| 55678 | 14912207 | 14914239 | 186104 |
| 55679 | 14925802 | 14927405 | 175868 |
| 55680 | 14996207 | 14998036 | 175868 |
| 55681 | 15096063 | 15096933 | 175868 |
| 55682 | 15141283 | 15142030 | 188487 |
| 55683 | 15144285 | 15146650 | 188487 |
| 55684 | 15215630 | 15218716 | 188487 |
| 55685 | 15221038 | 15221832 | 188487 |
| 55686 | 15503151 | 15504011 | 253072 |
| 55687 | 15515292 | 15516404 | |
| 55688 | 15518811 | 15519617 | |
| 55689 | 15671278 | 15672729 | 254695 |
| 55690 | 15683010 | 15689337 | 254695 |
| 55691 | 15694689 | 15695317 | 254695 |
| 55692 | 15696569 | 15698309 | 254695 |
| 55693 | 15709742 | 15710483 | 254695 |
| 55694 | 15725622 | 15726039 | 254695, 254645 |
| 55695 | 15728928 | 15730109 | 254645 |
| 55696 | 15968787 | 15970568 | |
| 55697 | 15989679 | 15991849 | 110693 |
| 55698 | 16115271 | 16116886 | 110693 |
| 55699 | 16135798 | 16137623 | 110693 |
| 55700 | 16175031 | 16180686 | 110693 |
| 55701 | 16203481 | 16209187 | 110693 |
| 55702 | 16356855 | 16357707 | 110693 |
| 55703 | 16625456 | 16629584 | 110693 |
| 55704 | 16759146 | 16761370 | 110693, 110696 |
| 55705 | 16796260 | 16797659 | |
| 55706 | 16806912 | 16811122 | 166689 |
| 55707 | 16812049 | 16813771 | 166689 |
| 55708 | 16825660 | 16826578 | 166689 |
| 55709 | 16836614 | 16837120 | 166689 |
| 55710 | 16839786 | 16840307 | 166689 |
| 55711 | 16865603 | 16866533 | 166689 |
| 55712 | 16902832 | 16903533 | 166689 |
| 55713 | 17017539 | 17018169 | 166689 |
| 55714 | 17018901 | 17019880 | 166689 |
| 55715 | 17033256 | 17037102 | 184669, 166689 |
| 55716 | 17098054 | 17100199 | 011405, 110700 |
| 55717 | 17218676 | 17219738 | 011405 |
| 55718 | 17228558 | 17230503 | 070081, 011405 |
| 55719 | 17297706 | 17299205 | 070081 |
| 55720 | 17372818 | 17374028 | 188211 |
| 55721 | 17374166 | 17376137 | 188211 |
| 55722 | 17410133 | 17411594 | 187486 |
| 55723 | 17426342 | 17428597 | 006071 |
| 55724 | 17435155 | 17435798 | 006071 |
| 55725 | 17704783 | 17706133 | |
| 55726 | 17772033 | 17773005 | 129159 |
| 55727 | 17791436 | 17792064 | 129159 |
| 55728 | 17975846 | 17976656 | 129158 |
| 55729 | 18033723 | 18035683 | 129158, 255448 |
| 55730 | 18126948 | 18127901 | 166788 |
| 55731 | 18245354 | 18246213 | |
| 55732 | 18256431 | 18259604 | 148965, 255071 |
| 55733 | 18270108 | 18270753 | 255071, 134339 |
| 55734 | 18280443 | 18281789 | |
| 55735 | 18287050 | 18287890 | 173432 |
| 55736 | 18330650 | 18333049 | 110756 |
| 55737 | 18342608 | 18344977 | 110768, 110756 |
| 55738 | 18404194 | 18406745 | |
| 55739 | 18415177 | 18417942 | 134333 |
| 55740 | 18478779 | 18479441 | 166800 |
| 55741 | 18547249 | 18549587 | 074319 |
| 55742 | 18609382 | 18611306 | 151116 |
| 55743 | 18654902 | 18657033 | 179119 |
| 55744 | 18719783 | 18721151 | 151117 |
| 55745 | 19049414 | 19050185 | |
| 55746 | 19138541 | 19139379 | 177054 |
| 55747 | 19262272 | 19262702 | 255308, 129173 |
| 55748 | 19297727 | 19299946 | 255308 |
| 55749 | 19367122 | 19368587 | |
| 55750 | 19386399 | 19393258 | 166833 |
| 55751 | 19393670 | 19397918 | 166833 |
| 55752 | 19400175 | 19403936 | 255270, 166833 |
| 55753 | 19411051 | 19411896 | 166833 |
| 55754 | 19419767 | 19421209 | 166833 |
| 55755 | 19440451 | 19442426 | 166833 |
| 55756 | 19449954 | 19451343 | 166833 |
| 55757 | 19459253 | 19460426 | 166833 |
| 55758 | 19463264 | 19464851 | 166833 |
| 55759 | 19468326 | 19469734 | 166833 |
| 55760 | 19528958 | 19530857 | 166833 |
| 55761 | 19531739 | 19534590 | 254622, 166833 |
| 55762 | 19544969 | 19548066 | 166833 |
| 55763 | 19559277 | 19560244 | 166833 |
| 55764 | 19576674 | 19577404 | 166833 |
| 55765 | 19617314 | 19619047 | 166833 |
| 55766 | 19680852 | 19682729 | 166833 |
| 55767 | 19689150 | 19691349 | 166833 |
| 55768 | 19700872 | 19702746 | 166833 |
| 55769 | 19704813 | 19706945 | 166833 |
| 55770 | 19718904 | 19721431 | 166833 |
| 55771 | 19732662 | 19738442 | 270607, 166833 |
| 55772 | 19744835 | 19746230 | 166833 |
| 55773 | 19746676 | 19747887 | 166833 |
| 55774 | 19788280 | 19788942 | 166833 |
| 55775 | 19797652 | 19799592 | 166833 |
| 55776 | 19811350 | 19811882 | 166833 |
| 55777 | 19822207 | 19823402 | 166833 |
| 55778 | 19842837 | 19843556 | 166833 |
| 55779 | 19850695 | 19851406 | 166833 |
| 55780 | 19885910 | 19886591 | 166833 |
| 55781 | 19911751 | 19912365 | 166833 |
| 55782 | 19974596 | 19975980 | 166833 |
| 55783 | 19976284 | 19976894 | 166833 |
| 55784 | 19997995 | 19998558 | 166833 |
| 55785 | 19999440 | 20003559 | 254542, 166833 |
| 55786 | 20015882 | 20018322 | 166833 |
| 55787 | 20021505 | 20026674 | 166833 |
| 55788 | 20029978 | 20035972 | 166833 |
| 55789 | 20041617 | 20042760 | 166833 |
| 55790 | 20043173 | 20047017 | 166833 |
| 55791 | 20054595 | 20059627 | 166833 |
| 55792 | 20062937 | 20064125 | 166833 |
| 55793 | 20078653 | 20079825 | 166833 |
| 55794 | 20081158 | 20081757 | 166833 |
| 55795 | 20132953 | 20135162 | 166833 |
| 55796 | 20384505 | 20386631 | 109854 |
| 55797 | 20408297 | 20410577 | 185238 |
| 55798 | 20539659 | 20540342 | |
| 55799 | 21930263 | 21931749 | |
| 55800 | 22097982 | 22098555 | |
| 55801 | 22213770 | 22215531 | 171714 |
| 55802 | 22645958 | 22648247 | 229387, 148935, 183161 |
| 55803 | 22656329 | 22657061 | 148935 |
| 55804 | 22687638 | 22689070 | 148935 |
| 55805 | 22695308 | 22699657 | 148935 |
| 55806 | 22701227 | 22703969 | 148935 |
| 55807 | 22723986 | 22725466 | 148935 |
| 55808 | 22850715 | 22851942 | 246225, 198168 |
| 55809 | 22971552 | 22972198 | |
| 55810 | 22997693 | 22998407 | |
| 55811 | 23032400 | 23032922 | |
| 55812 | 23134348 | 23134952 | |
| 55813 | 25086318 | 25088300 | 187398 |
| 55814 | 25115227 | 25116293 | |
| 55815 | 26164682 | 26166450 | |
| 55816 | 26208664 | 26210730 | 255243 |
| 55817 | 26931030 | 26931490 | |
| 55818 | 26966256 | 26970769 | |
| 55819 | 26971375 | 26972856 | |
| 55820 | 27013266 | 27014466 | |
| 55821 | 27015392 | 27016564 | 176971 |
| 55822 | 27046741 | 27047463 | |
| 55823 | 27076362 | 27077775 | 129151, 254560 |
| 55824 | 27079986 | 27080403 | 129151, 254560 |
| 55825 | 27109756 | 27110525 | 129151, 254560 |
| 55826 | 27115524 | 27116304 | 129151, 254560 |
| 55827 | 27119018 | 27119826 | 129151, 254560 |
| 55828 | 27222857 | 27223933 | 254560 |
| 55829 | 27229926 | 27231663 | 254560 |
| 55830 | 27303649 | 27304751 | |
| 55831 | 27313081 | 27314573 | |
| 55832 | 27333594 | 27334301 | |
| 55833 | 27395712 | 27396347 | 205213 |
| 55834 | 27402418 | 27403185 | 205213 |
| 55835 | 27413138 | 27414375 | 205213 |
| 55836 | 27416186 | 27418739 | 205213 |
| 55837 | 27422796 | 27424605 | 205213 |
| 55838 | 27442598 | 27443959 | 205213 |
| 55839 | 27444405 | 27445237 | 205213 |
| 55840 | 27477028 | 27478259 | 205213 |
| 55841 | 27489046 | 27495152 | 254862, 205213 |
| 55842 | 27527215 | 27529323 | 245573, 148943 |
| 55843 | 27535586 | 27536668 | 245573 |
| 55844 | 28131514 | 28132623 | 169519 |
| 55845 | 28167582 | 28168221 | 169519 |
| 55846 | 28190091 | 28190996 | 169519 |
| 55847 | 28778350 | 28779635 | 249867 |
| 55848 | 29282292 | 29283870 | 254526 |
| 55849 | 29303513 | 29304704 | |
| 55850 | 29314486 | 29316402 | |
| 55851 | 29317500 | 29319273 | |
| 55852 | 29342441 | 29344137 | |
| 55853 | 29837769 | 29838633 | 254734 |
| 55854 | 30344173 | 30345748 | 152219 |
| 55855 | 30566844 | 30567754 | 066382 |
| 55856 | 30607331 | 30608222 | 066382, 254489 |
| 55857 | 30822310 | 30823782 | |
| 55858 | 31005976 | 31006707 | 170959 |
| 55859 | 31012017 | 31015157 | 170959 |
| 55860 | 31390880 | 31392095 | 170946, 170959 |
| 55861 | 31530249 | 31532621 | 109911, 148950 |
| 55862 | 31667060 | 31668305 | 109911, 228061 |
| 55863 | 31676943 | 31677479 | 109911, 228061 |
| 55864 | 31924774 | 31925593 | 049449 |
| 55865 | 32109445 | 32110367 | 049449 |
| 55866 | 32111916 | 32113870 | 049449 |
| 55867 | 32604853 | 32606144 | 149100 |
| 55868 | 32720024 | 32720594 | 186714 |
| 55869 | 32728049 | 32729810 | 186714 |
| 55870 | 32850601 | 32852499 | 135378 |
| 55871 | 32913041 | 32916189 | 060749 |
| 55872 | 33032886 | 33033893 | |
| 55873 | 33035263 | 33035708 | |
| 55874 | 33036724 | 33040111 | 121690 |
| 55875 | 33060328 | 33062255 | 176148 |
| 55876 | 33081672 | 33084480 | 176148 |
| 55877 | 33181333 | 33183367 | 247151, 176102 |
| 55878 | 33278029 | 33281121 | 110422 |
| 55879 | 33499402 | 33500013 | |
| 55880 | 33709775 | 33715533 | 255202 |
| 55881 | 33715808 | 33717688 | 255202 |
| 55882 | 33720446 | 33726158 | 205177, 085063 |
| 55883 | 33729473 | 33730020 | 085063 |
| 55884 | 33756047 | 33758904 | 085063 |
| 55885 | 33795052 | 33796548 | 254508, 110429 |
| 55886 | 33801443 | 33802219 | |
| 55887 | 34049052 | 34050563 | |
| 55888 | 34057786 | 34058456 | |
| 55889 | 34072112 | 34075893 | 135387 |
| 55890 | 34126179 | 34128518 | 135372 |
| 55891 | 34195889 | 34196704 | 166016 |
| 55892 | 34214608 | 34215082 | 166016 |
| 55893 | 34230018 | 34230903 | 166016 |
| 55894 | 34234118 | 34238126 | 166016 |
| 55895 | 34255408 | 34256410 | 166016 |
| 55896 | 34259610 | 34260560 | 166016 |
| 55897 | 34263227 | 34266745 | 166016 |
| 55898 | 34284904 | 34286489 | 166016 |
| 55899 | 34296144 | 34297009 | 166016 |
| 55900 | 34329471 | 34331906 | 166016 |
| 55901 | 34337610 | 34341533 | 166016 |
| 55902 | 34366165 | 34368420 | 166016 |
| 55903 | 34371394 | 34371954 | 166016 |
| 55904 | 34375760 | 34377038 | 166016 |
| 55905 | 34377557 | 34381561 | 166016 |
| 55906 | 34392182 | 34393421 | |
| 55907 | 34424884 | 34425504 | |
| 55908 | 34427381 | 34428450 | |
| 55909 | 34429855 | 34430664 | |
| 55910 | 34436688 | 34437813 | |
| 55911 | 34448303 | 34449853 | |
| 55912 | 34459983 | 34461942 | 121691 |
| 55913 | 34522990 | 34524153 | 135374 |
| 55914 | 34621962 | 34622853 | |
| 55915 | 34652859 | 34654577 | 135373 |
| 55916 | 34661173 | 34662458 | 135373 |
| 55917 | 34674662 | 34677851 | 135373 |
| 55918 | 34702551 | 34703328 | |
| 55919 | 34706719 | 34707506 | |
| 55920 | 34804712 | 34806853 | |
| 55921 | 34931373 | 34932018 | 149089 |
| 55922 | 34936691 | 34939311 | 110435, 149089 |
| 55923 | 34951045 | 34951966 | 110435 |
| 55924 | 35257456 | 35258039 | |
| 55925 | 35324546 | 35327421 | 110436 |
| 55926 | 35357794 | 35362532 | 110436 |
| 55927 | 35379326 | 35380241 | 110436 |
| 55928 | 35380603 | 35382386 | 110436 |
| 55929 | 35385053 | 35386448 | 110436 |
| 55930 | 35394506 | 35395670 | 110436 |
| 55931 | 35412351 | 35414233 | 110436 |
| 55932 | 35639302 | 35642281 | 179431 |
| 55933 | 35650795 | 35652755 | |
| 55934 | 35683023 | 35685960 | 166326 |
| 55935 | 35756110 | 35756810 | 166326 |
| 55936 | 35771805 | 35772351 | 166326 |
| 55937 | 35819813 | 35821100 | 166326 |
| 55938 | 35948418 | 35949816 | |
| 55939 | 35964993 | 35966218 | 179241 |
| 55940 | 36169561 | 36173395 | 179241 |
| 55941 | 36287254 | 36289108 | |
| 55942 | 36310210 | 36311524 | 110442 |
| 55943 | 36521353 | 36522868 | 175104 |
| 55944 | 36530177 | 36532657 | 166349, 175104 |
| 55945 | 36615723 | 36616807 | 166352, 175097 |
| 55946 | 37269571 | 37270149 | |
| 55947 | 37343999 | 37344902 | |
| 55948 | 37521351 | 37527919 | |
| 55949 | 40349866 | 40351895 | 148948 |
| 55950 | 40381689 | 40382290 | 148948 |
| 55951 | 40408528 | 40410966 | 148948 |
| 55952 | 40424676 | 40425962 | 148948 |
| 55953 | 40456733 | 40457484 | 148948 |
| 55954 | 40535507 | 40536089 | 148948 |
| 55955 | 40711326 | 40711804 | 148948 |
| 55956 | 40737803 | 40738912 | 148948 |
| 55957 | 41120993 | 41122048 | 148948 |
| 55958 | 41143128 | 41143668 | 148948 |
| 55959 | 41399042 | 41399999 | 148948 |
| 55960 | 41404420 | 41405021 | 148948 |
| 55961 | 43333111 | 43334462 | 166181 |
| 55962 | 43379814 | 43381767 | 052841, 254907 |
| 55963 | 43424749 | 43426526 | 052841 |
| 55964 | 43665091 | 43666694 | 149084 |
| 55965 | 43701725 | 43703726 | 149084 |
| 55966 | 43852182 | 43853624 | 149084, 246250 |
| 55967 | 43890457 | 43891018 | 246250 |
| 55968 | 43901972 | 43903250 | 166199, 246250 |
| 55969 | 43941267 | 43943520 | 254409, 244926, 166199 |
| 55970 | 44006071 | 44007062 | 254409 |
| 55971 | 44008817 | 44009600 | 254409 |
| 55972 | 44087017 | 44088667 | 110455 |
| 55973 | 44116991 | 44118564 | 151348 |
| 55974 | 44265119 | 44265807 | 151348 |
| 55975 | 44326688 | 44327803 | 052850 |
| 55976 | 44585586 | 44587656 | 085117 |
| 55977 | 44589691 | 44591300 | 085117 |
| 55978 | 44605914 | 44606948 | 085117 |
| 55979 | 44655831 | 44657002 | |
| 55980 | 44730314 | 44731346 | |
| 55981 | 45000316 | 45000952 | |
| 55982 | 45001324 | 45002083 | |
| 55983 | 45110665 | 45111289 | |
| 55984 | 45167363 | 45169562 | 019485 |
| 55985 | 45201436 | 45203217 | 019485 |
| 55986 | 45412876 | 45413366 | 255041 |
| 55987 | 45415280 | 45416387 | 255041 |
| 55988 | 45757230 | 45759365 | 255426, 254519 |
| 55989 | 45792633 | 45793998 | 205106 |
| 55990 | 45798579 | 45799543 | |
| 55991 | 45812917 | 45813703 | |
| 55992 | 45820124 | 45821393 | |
| 55993 | 45822899 | 45824909 | |
| 55994 | 45825199 | 45827901 | 181830 |
| 55995 | 45864639 | 45865988 | |
| 55996 | 45868437 | 45871027 | 121671 |
| 55997 | 45873381 | 45874010 | 121671 |
| 55998 | 45879744 | 45882302 | 121671 |
| 55999 | 45906637 | 45907588 | 121653 |
| 56000 | 45921115 | 45921930 | 121653 |
| 56001 | 45934858 | 45935581 | 121680 |
| 56002 | 45938412 | 45940500 | 121680 |
| 56003 | 46140727 | 46144902 | 135365 |
| 56004 | 46263842 | 46265733 | |
| 56005 | 46270057 | 46271249 | |
| 56006 | 46295209 | 46296062 | 255007 |
| 56007 | 46368807 | 46370059 | 149091 |
| 56008 | 46401931 | 46403483 | 110492, 149091 |
| 56009 | 46532881 | 46533369 | 110497 |
| 56010 | 46574630 | 46576943 | 110497 |
| 56011 | 46614524 | 46616434 | 110497 |
| 56012 | 46637915 | 46640258 | 175224, 180423 |
| 56013 | 46655185 | 46655832 | 175224 |
| 56014 | 46695738 | 46698027 | 175224 |
| 56015 | 46704731 | 46705114 | 175220 |
| 56016 | 46717296 | 46718260 | 175220 |
| 56017 | 46721198 | 46723662 | 175213, 175220 |
| 56018 | 46731859 | 46733888 | |
| 56019 | 46734038 | 46737319 | |
| 56020 | 46739221 | 46743624 | 180210 |
| 56021 | 46766470 | 46767209 | 175216 |
| 56022 | 46847764 | 46848639 | 175216 |
| 56023 | 46867131 | 46868054 | 247675, 175216 |
| 56024 | 46957961 | 46958949 | 149179 |
| 56025 | 47026166 | 47026774 | 149179 |
| 56026 | 47120858 | 47121462 | 149179 |
| 56027 | 47197373 | 47199950 | 165912, 149182 |
| 56028 | 47206099 | 47209702 | 165912 |
| 56029 | 47210451 | 47211576 | |
| 56030 | 47235577 | 47238469 | 134574 |
| 56031 | 47254291 | 47257566 | 134574 |
| 56032 | 47262681 | 47265502 | 134575 |
| 56033 | 47265997 | 47267869 | 134575 |
| 56034 | 47269289 | 47271484 | 025434, 134575 |
| 56035 | 47276418 | 47277153 | 025434 |
| 56036 | 47278541 | 47280472 | 025434 |
| 56037 | 47290403 | 47292301 | 110514, 256746 |
| 56038 | 47328938 | 47329301 | 110514 |
| 56039 | 47376236 | 47377358 | 066336 |
| 56040 | 47428726 | 47431508 | 255197, 165915 |
| 56041 | 47446859 | 47449332 | 165916 |
| 56042 | 47452186 | 47453751 | |
| 56043 | 47455556 | 47456788 | |
| 56044 | 47520073 | 47520981 | 149187 |
| 56045 | 47544839 | 47545841 | 149187 |
| 56046 | 47569063 | 47569655 | 149187 |
| 56047 | 47573334 | 47575297 | 149187 |
| 56048 | 47586313 | 47588023 | 213619, 110536, 149187 |
| 56049 | 47594232 | 47594714 | 123444, 213619, 110536 |
| 56050 | 47599693 | 47601501 | 123444, 231880, 213619 |
| 56051 | 47663073 | 47664669 | 109919 |
| 56052 | 47735932 | 47737072 | 165923 |
| 56053 | 47745453 | 47746367 | 109920 |
| 56054 | 47787093 | 47790280 | 109920 |
| 56055 | 47869003 | 47870748 | 030066 |
| 56056 | 47945656 | 47946475 | |
| 56057 | 47958885 | 47960311 | |
| 56058 | 47963601 | 47964316 | |
| 56059 | 47967959 | 47969223 | |
| 56060 | 48000873 | 48004080 | 149177 |
| 56061 | 48010896 | 48013900 | 149177 |
| 56062 | 48014802 | 48015515 | 149177 |
| 56063 | 48018469 | 48021627 | 149177 |
| 56064 | 48034698 | 48035505 | 149177 |
| 56065 | 48082806 | 48083419 | 149177 |
| 56066 | 48084869 | 48086198 | 149177 |
| 56067 | 48102281 | 48103702 | 149177 |
| 56068 | 48114841 | 48116500 | 149177 |
| 56069 | 48122308 | 48124416 | 149177 |
| 56070 | 48125144 | 48125959 | 149177 |
| 56071 | 49229342 | 49230307 | 086205 |
| 56072 | 56391960 | 56393021 | |
| 56073 | 56934528 | 56935183 | |
| 56074 | 57007199 | 57007927 | |
| 56075 | 57044554 | 57045108 | |
| 56076 | 57045157 | 57046251 | |
| 56077 | 57056316 | 57057195 | |
| 56078 | 57060276 | 57060924 | |
| 56079 | 57073703 | 57074364 | 149115 |
| 56080 | 57077397 | 57079947 | 149115 |
| 56081 | 57088577 | 57090581 | 149115 |
| 56082 | 57091093 | 57094052 | 149136, 254662, 149115 |
| 56083 | 57102400 | 57104070 | 149136 |
| 56084 | 57117021 | 57117742 | 109991 |
| 56085 | 57191483 | 57196165 | 254979, 134802 |
| 56086 | 57201938 | 57202782 | |
| 56087 | 57205871 | 57206687 | |
| 56088 | 57209096 | 57209723 | |
| 56089 | 57220827 | 57221745 | |
| 56090 | 57224605 | 57228910 | 186907 |
| 56091 | 57243182 | 57244931 | 255301, 186907 |
| 56092 | 57247463 | 57248815 | |
| 56093 | 57264709 | 57266487 | 149150 |
| 56094 | 57269320 | 57270828 | 149150 |
| 56095 | 57281372 | 57283917 | 149150 |
| 56096 | 57297467 | 57299238 | 134809 |
| 56097 | 57333300 | 57336194 | 156587 |
| 56098 | 57336530 | 57337250 | |
| 56099 | 57363913 | 57368252 | 149131 |
| 56100 | 57383831 | 57384596 | |
| 56101 | 57385679 | 57386917 | |
| 56102 | 57405477 | 57406620 | 254602 |
| 56103 | 57406935 | 57407783 | 254602 |
| 56104 | 57424575 | 57425819 | 172409 |
| 56105 | 57433801 | 57436657 | 156599 |
| 56106 | 57447219 | 57447683 | 156599 |
| 56107 | 57479191 | 57480976 | 213593, 254462, 156603 |
| 56108 | 57508382 | 57510130 | 254732, 211450, 213593, |
| | | | 254462 |
| 56109 | 57528542 | 57531132 | 254732, 198561, 254462 |
| 56110 | 57544804 | 57547116 | 254732, 198561, 254462 |
| 56111 | 57560268 | 57561211 | 254732, 198561 |
| 56112 | 57566264 | 57567358 | 198561 |
| 56113 | 57568006 | 57568718 | 198561 |
| 56114 | 57826134 | 57826961 | 255146, 186509 |
| 56115 | 58340507 | 58348230 | 255073, 186660, 110031 |
| 56116 | 58394576 | 58396240 | |
| 56117 | 58421420 | 58422687 | 149124 |
| 56118 | 58426942 | 58427314 | 149124 |
| 56119 | 58498935 | 58499970 | 149124 |
| 56120 | 58529699 | 58531067 | |
| 56121 | 58555170 | 58556380 | |
| 56122 | 58647876 | 58648792 | 254717, 156689 |
| 56123 | 58673351 | 58674281 | 166840, 224130 |
| 56124 | 58690583 | 58691242 | 166840, 255523 |
| 56125 | 58693568 | 58696991 | 255523, 166840 |
| 56126 | 58706576 | 58708513 | 255240, 166840 |
| 56127 | 58708983 | 58709921 | 255240, 166840 |
| 56128 | 58712875 | 58714234 | 255240, 166840 |
| 56129 | 58766183 | 58767995 | 254786, 255240, 166840 |
| 56130 | 58780245 | 58780933 | 255240, 166840 |
| 56131 | 58792338 | 58793287 | 255240, 166840 |
| 56132 | 58909711 | 58910996 | 166801, 245571, 255381 |
| 56133 | 58938784 | 58944931 | 110042 |
| 56134 | 58958352 | 58959668 | 110042 |
| 56135 | 58968675 | 58969298 | 110042 |
| 56136 | 59304349 | 59305867 | |
| 56137 | 59317607 | 59319618 | |
| 56138 | 59323266 | 59324489 | |
| 56139 | 59324532 | 59325292 | |
| 56140 | 59327436 | 59328560 | |
| 56141 | 59333132 | 59334242 | 255008 |
| 56142 | 59378876 | 59380070 | 110048 |
| 56143 | 59381736 | 59384614 | 255139, 110048 |
| 56144 | 59410844 | 59411510 | 166889 |
| 56145 | 59435088 | 59438209 | 255355, 166889 |
| 56146 | 59441662 | 59442293 | 255355 |
| 56147 | 59443461 | 59444120 | 255355 |
| 56148 | 59521316 | 59524351 | 254477, 166900 |
| 56149 | 59543840 | 59546344 | 166900 |
| 56150 | 59554306 | 59554965 | 166900 |
| 56151 | 59577536 | 59578685 | 166902 |
| 56152 | 59936512 | 59937547 | |
| 56153 | 60425834 | 60426435 | 181995 |
| 56154 | 60608777 | 60610974 | 256813, 110104, 255959 |
| 56155 | 60619332 | 60620691 | 183134 |
| 56156 | 60639028 | 60640234 | 149506 |
| 56157 | 60672408 | 60674484 | 257052, 110107 |
| 56158 | 60685651 | 60686528 | 256196, 110108 |
| 56159 | 60691481 | 60692820 | 006118, 256196 |
| 56160 | 60718645 | 60720526 | 110446 |
| 56161 | 60764057 | 60764814 | 013725 |
| 56162 | 60919770 | 60921740 | 167987 |
| 56163 | 60925576 | 60930010 | 167987 |
| 56164 | 61061252 | 61063063 | 167992 |
| 56165 | 61065059 | 61065716 | |
| 56166 | 61099558 | 61103332 | 149476, 167986 |
| 56167 | 61114883 | 61116571 | 162144, 149476 |
| 56168 | 61128418 | 61130519 | 149483, 162144 |
| 56169 | 61136711 | 61137723 | 149483 |
| 56170 | 61146115 | 61146600 | |
| 56171 | 61159219 | 61160886 | 187049 |
| 56172 | 61196326 | 61198387 | 256591, 167985, 149532 |
| 56173 | 61213559 | 61214119 | 256591, 167985 |
| 56174 | 61256140 | 61256872 | 162148 |
| 56175 | 61296740 | 61298368 | 011347 |
| 56176 | 61298398 | 61298810 | 011347 |
| 56177 | 61311226 | 61311960 | 011347 |
| 56178 | 61343465 | 61344168 | 011347 |
| 56179 | 61347729 | 61349205 | 011347 |
| 56180 | 61519706 | 61521606 | 124920, 124915 |
| 56181 | 61522841 | 61525823 | 124915, 124920 |
| 56182 | 61532320 | 61535006 | 124920 |
| 56183 | 61558932 | 61561119 | 207601, 134824, 168496, |
| | | | 134825 |
| 56184 | 61582054 | 61585212 | 222326, 149485, 134824 |
| 56185 | 61587026 | 61589393 | 149485, 134824 |
| 56186 | 61594251 | 61597413 | 149485, 134824 |
| 56187 | 61655292 | 61656241 | 221968 |
| 56188 | 61657938 | 61659739 | 221968 |
| 56189 | 61722310 | 61723725 | 167995 |
| 56190 | 61728791 | 61732223 | 167995, 167996 |
| 56191 | 61732480 | 61745414 | 269089, 167995, 167996 |
| 56192 | 61748060 | 61749236 | |
| 56193 | 61762508 | 61762953 | |
| 56194 | 61767949 | 61769707 | |
| 56195 | 61792338 | 61793924 | |
| 56196 | 62165487 | 62166531 | |
| 56197 | 62192741 | 62193330 | 255446 |
| 56198 | 62200102 | 62200838 | |
| 56199 | 62216446 | 62217514 | 124942 |
| 56200 | 62309646 | 62315889 | 257058, 124942 |
| 56201 | 62319901 | 62322803 | 124942 |
| 56202 | 62327782 | 62328596 | 254772, 255508 |
| 56203 | 62340190 | 62342953 | 149016, 254772, 255508 |
| 56204 | 62354449 | 62356566 | 149016, 255508 |
| 56205 | 62358085 | 62360300 | 149016, 255508 |
| 56206 | 62368005 | 62369514 | 149480 |
| 56207 | 62379153 | 62380855 | 149489, 149499 |
| 56208 | 62388491 | 62390768 | 149541 |
| 56209 | 62408149 | 62408629 | 089597 |
| 56210 | 62413107 | 62414556 | 185085, 089597 |
| 56211 | 62419854 | 62421401 | 185085, 255432 |
| 56212 | 62431876 | 62434268 | 206597, 214756, 255432, |
| | | | 162194 |
| 56213 | 62438382 | 62440103 | 162194, 204922 |
| 56214 | 62445689 | 62447178 | 162191 |
| 56215 | 62476143 | 62478087 | 162188, 168000, 234857 |
| 56216 | 62479956 | 62482462 | 214753, 234857 |
| 56217 | 62493277 | 62494869 | 234857, 214753 |
| 56218 | 62495315 | 62496944 | 162222 |
| 56219 | 62528833 | 62529831 | 168002 |
| 56220 | 62538160 | 62539621 | 267811, 168569, 162227 |
| 56221 | 62553872 | 62555850 | 185475, 269176, 162227, |
| | | | 168569 |
| 56222 | 62558420 | 62559718 | 162231, 168569 |
| 56223 | 62571974 | 62573420 | 162231 |
| 56224 | 62597955 | 62600856 | 133316, 256690, 162236 |
| 56225 | 62606164 | 62610524 | 222328, 133316 |
| 56226 | 62622419 | 62624527 | 168003, 255717 |
| 56227 | 62647346 | 62651205 | 168003 |
| 56228 | 63008125 | 63009384 | 184999 |
| 56229 | 63013047 | 63015874 | 184999 |
| 56230 | 63017363 | 63018455 | 184999 |
| 56231 | 63056836 | 63058043 | 184999 |
| 56232 | 63136617 | 63138322 | 149742, 184999 |
| 56233 | 63171841 | 63172990 | 149742 |
| 56234 | 63272330 | 63272883 | |
| 56235 | 63277761 | 63278302 | 133317 |
| 56236 | 63303818 | 63305236 | 133321 |
| 56237 | 63334442 | 63335904 | |
| 56238 | 63341944 | 63342982 | 176485 |
| 56239 | 63376379 | 63377760 | 176485 |
| 56240 | 63380751 | 63382378 | 176485 |
| 56241 | 63437366 | 63439553 | 184743 |
| 56242 | 63448584 | 63450238 | 133318 |
| 56243 | 63530103 | 63533148 | 188070 |
| 56244 | 63535043 | 63537038 | 255651, 188070 |
| 56245 | 63580515 | 63581652 | 168005 |
| 56246 | 63605845 | 63607862 | 072518 |
| 56247 | 63612650 | 63613198 | 072518 |
| 56248 | 63626084 | 63626574 | 072518 |
| 56249 | 63639391 | 63640127 | 072518 |
| 56250 | 63655672 | 63657281 | 072518 |
| 56251 | 63685356 | 63685916 | |
| 56252 | 63706154 | 63707324 | 110583 |
| 56253 | 63741748 | 63742881 | 256100, 176340 |
| 56254 | 63753425 | 63754875 | 256100, 167770 |
| 56255 | 63852519 | 63853298 | 133315, 256481, 256824 |
| 56256 | 63932281 | 63934507 | 133315 |
| 56257 | 63952143 | 63954583 | 168439 |
| 56258 | 63992888 | 63994547 | 149761, 149743 |
| 56259 | 63997672 | 63998809 | 110011, 149761, 256116 |
| 56260 | 64004851 | 64005462 | 173511 |
| 56261 | 64007506 | 64010378 | 173486 |
| 56262 | 64012336 | 64015500 | 256940, 257086, 173457 |
| 56263 | 64018499 | 64019221 | 149782 |
| 56264 | 64026901 | 64027667 | 149782 |
| 56265 | 64035850 | 64038566 | 002330, 173264, 149782 |
| 56266 | 64038626 | 64039766 | 002330, 173264 |
| 56267 | 64040751 | 64041454 | 002330, 173264 |
| 56268 | 64051456 | 64053463 | 002330, 173264 |
| 56269 | 64068038 | 64068870 | 219435, 257069 |
| 56270 | 64071489 | 64074877 | 173153, 219435, 257069 |
| 56271 | 64077366 | 64077884 | 173153 |
| 56272 | 64084358 | 64087132 | 126432, 173113 |
| 56273 | 64099089 | 64100426 | |
| 56274 | 64102413 | 64103198 | |
| 56275 | 64122499 | 64123439 | 168071 |
| 56276 | 64125428 | 64127434 | 162302 |
| 56277 | 64321994 | 64323515 | 168065 |
| 56278 | 64333762 | 64334400 | 168065 |
| 56279 | 64545140 | 64547429 | 269038, 168066 |
| 56280 | 64577320 | 64578745 | 133895 |
| 56281 | 64616076 | 64617169 | |
| 56282 | 64621456 | 64625738 | 110047 |
| 56283 | 64630924 | 64633431 | 110047 |
| 56284 | 64636278 | 64639028 | 110047 |
| 56285 | 64640034 | 64640821 | 110047 |
| 56286 | 64644314 | 64647546 | 110047 |
| 56287 | 64655067 | 64657204 | 110047 |
| 56288 | 64658199 | 64659504 | 207648, 229719, 267804 |
| 56289 | 64659741 | 64662210 | 110046, 229719 |
| 56290 | 64682895 | 64685053 | 068971, 110046 |
| 56291 | 64691334 | 64692918 | 068971 |
| 56292 | 64763776 | 64765096 | 168062 |
| 56293 | 64780842 | 64782640 | 213465, 273003 |
| 56294 | 64794574 | 64795657 | 110025, 273003 |
| 56295 | 64808234 | 64809673 | 168061 |
| 56296 | 64836658 | 64837441 | 146670 |
| 56297 | 64851154 | 64852660 | 162300, 187066, 146670 |
| 56298 | 64863133 | 64864899 | 149823 |
| 56299 | 64869402 | 64870305 | 149823 |
| 56300 | 64877678 | 64878406 | 254501, 149823 |
| 56301 | 64879068 | 64881633 | 149809, 254501, 149823 |
| 56302 | 64883566 | 64885651 | 174276, 255173, 149809 |
| 56303 | 64888547 | 64890419 | 149792, 162298, 149806 |
| 56304 | 64900780 | 64903544 | 162298 |
| 56305 | 64909391 | 64910059 | |
| 56306 | 64918034 | 64919191 | |
| 56307 | 64932623 | 64934799 | |
| 56308 | 64948074 | 64950456 | 254614, 014216 |
| 56309 | 64993004 | 64993659 | 197847 |
| 56310 | 65028465 | 65030081 | 014138 |
| 56311 | 65042445 | 65043783 | 014138 |
| 56312 | 65047091 | 65047707 | 014138 |
| 56313 | 65081778 | 65083568 | 149798 |
| 56314 | 65100972 | 65102402 | 133884 |
| 56315 | 65125100 | 65125944 | |
| 56316 | 65137929 | 65139199 | 255478 |
| 56317 | 65147969 | 65151198 | 162241 |
| 56318 | 65153186 | 65155043 | 126391 |
| 56319 | 65183756 | 65201046 | 245532 |
| 56320 | 65201104 | 65206569 | 245532 |
| 56321 | 65208647 | 65210098 | 245532 |
| 56322 | 65210458 | 65215211 | 245532 |
| 56323 | 65215554 | 65217160 | |
| 56324 | 65221974 | 65222952 | 173727 |
| 56325 | 65243496 | 65247365 | |
| 56326 | 65258707 | 65262521 | |
| 56327 | 65263934 | 65275567 | 270117, 251562 |
| 56328 | 65292279 | 65293857 | 142186 |
| 56329 | 65307470 | 65308805 | 168056 |
| 56330 | 65312979 | 65314375 | 168056 |
| 56331 | 65323135 | 65323731 | 168056 |
| 56332 | 65324533 | 65326909 | 168056 |
| 56333 | 65336406 | 65338935 | 260233, 173465 |
| 56334 | 65339274 | 65341044 | 176973, 173465 |
| 56335 | 65341472 | 65342476 | 176973 |
| 56336 | 65342483 | 65344122 | 173442 |
| 56337 | 65344572 | 65345186 | 173442 |
| 56338 | 65380496 | 65384261 | 197136, 173327 |
| 56339 | 65414211 | 65415422 | 213445 |
| 56340 | 65419656 | 65421464 | 173039 |
| 56341 | 65428949 | 65431598 | 173039 |
| 56342 | 65478682 | 65480404 | 172977 |
| 56343 | 65487411 | 65488522 | 172922 |
| 56344 | 65547315 | 65548827 | 175827, 254470 |
| 56345 | 65585002 | 65586298 | |
| 56346 | 65623921 | 65629446 | 172732, 172803, 172757 |
| 56347 | 65640823 | 65641528 | 172638 |
| 56348 | 65655158 | 65656946 | 172500 |
| 56349 | 65657721 | 65659143 | 175602 |
| 56350 | 65665684 | 65668293 | 175592 |
| 56351 | 65669606 | 65671096 | |
| 56352 | 65679959 | 65681239 | |
| 56353 | 65682009 | 65684938 | 175573 |
| 56354 | 65685292 | 65688116 | 175550, 175573 |
| 56355 | 65728201 | 65730339 | 175467 |
| 56356 | 65768993 | 65770775 | 175334, 175376 |
| 56357 | 65818811 | 65820836 | 238752, 087365 |
| 56358 | 65822890 | 65823636 | 087365 |
| 56359 | 65836253 | 65838579 | 175115, 087365, 255038 |
| 56360 | 65847514 | 65848298 | 175115 |
| 56361 | 65880050 | 65881146 | 175115 |
| 56362 | 65886138 | 65887881 | 175115 |
| 56363 | 65903563 | 65905616 | 175115 |
| 56364 | 65909561 | 65910972 | 175115 |
| 56365 | 65989842 | 65990582 | 175115 |
| 56366 | 66024618 | 66026200 | 174996 |
| 56367 | 66034957 | 66037123 | 174903, 254762, 174996 |
| 56368 | 66045032 | 66045788 | 174871, 245156 |
| 56369 | 66055583 | 66057504 | 174851 |
| 56370 | 66103907 | 66104911 | 174744, 174791, 254756 |
| 56371 | 66111629 | 66113031 | 174684, 174744 |
| 56372 | 66115242 | 66116221 | 255468 |
| 56373 | 66118284 | 66119373 | 255468 |
| 56374 | 66188964 | 66189561 | 174576 |
| 56375 | 66205300 | 66206894 | 174547 |
| 56376 | 66246347 | 66248540 | 254986, 255517 |
| 56377 | 66277717 | 66278702 | 174483, 256349 |
| 56378 | 66312706 | 66313962 | 248746, 174165 |
| 56379 | 66360064 | 66361181 | 173992, 182791 |
| 56380 | 66374179 | 66375561 | |
| 56381 | 66383111 | 66385600 | 248643, 239306, 173933 |
| 56382 | 66405137 | 66407181 | 248643, 173933 |
| 56383 | 66418398 | 66419214 | 173933 |
| 56384 | 66444698 | 66446368 | 173914 |
| 56385 | 66477189 | 66477690 | 173898 |
| 56386 | 66485888 | 66486351 | 173898 |
| 56387 | 66494088 | 66496525 | 173898 |
| 56388 | 66511742 | 66513208 | 173715 |
| 56389 | 66609567 | 66611817 | 173653, 173715 |
| 56390 | 66658784 | 66661135 | 173599 |
| 56391 | 66669242 | 66670025 | 173599 |
| 56392 | 66672918 | 66676103 | 173599 |
| 56393 | 66702235 | 66702709 | 173599 |
| 56394 | 66703852 | 66704312 | 173599 |
| 56395 | 66717444 | 66718992 | 173599 |
| 56396 | 66724076 | 66726490 | 173599 |
| 56397 | 66734730 | 66739863 | |
| 56398 | 66770057 | 66771020 | |
| 56399 | 66789671 | 66790776 | 173227 |
| 56400 | 66820068 | 66825778 | 173156 |
| 56401 | 66826820 | 66828752 | 173156 |
| 56402 | 66881940 | 66883193 | |
| 56403 | 66884914 | 66888360 | 173120 |
| 56404 | 66957863 | 66958172 | 173120 |
| 56405 | 67007023 | 67009276 | 173120 |
| 56406 | 67015721 | 67016748 | 173120 |
| 56407 | 67033070 | 67035556 | 173020 |
| 56408 | 67055160 | 67056646 | 172932 |
| 56409 | 67069749 | 67071937 | 172830, 172932 |
| 56410 | 67118610 | 67119136 | 256514, 175482 |
| 56411 | 67119767 | 67124840 | 175482, 256514 |
| 56412 | 67139230 | 67143292 | 175505, 256514 |
| 56413 | 67158596 | 67159816 | 172613 |
| 56414 | 67168346 | 67169592 | 172531 |
| 56415 | 67187948 | 67188759 | 172531, 172508 |
| 56416 | 67195016 | 67196976 | 175634 |
| 56417 | 67210015 | 67211510 | 172725 |
| 56418 | 67231071 | 67233294 | 172663 |
| 56419 | 67235550 | 67237275 | 172663 |
| 56420 | 67249911 | 67251055 | 110711 |
| 56421 | 67271929 | 67274120 | 167797, 110697 |
| 56422 | 67274494 | 67276508 | 167797 |
| 56423 | 67330448 | 67332261 | |
| 56424 | 67346474 | 67347644 | |
| 56425 | 67373535 | 67375358 | 167792, 255119, 184224 |
| 56426 | 67396159 | 67398764 | 167799 |
| 56427 | 67399450 | 67400544 | 167800 |
| 56428 | 67417403 | 67419217 | 227834, 132744 |
| 56429 | 67770784 | 67771835 | 110057 |
| 56430 | 67777209 | 67778751 | 006534 |
| 56431 | 67797628 | 67799078 | 110717, 255306 |
| 56432 | 67800217 | 67802212 | 266737, 110717 |
| 56433 | 67803633 | 67808820 | 110719, 110717 |
| 56434 | 67866821 | 67867515 | 110721 |
| 56435 | 67887467 | 67891655 | 255236, 110721 |
| 56436 | 67894192 | 67896690 | 255236 |
| 56437 | 67905055 | 67905978 | |
| 56438 | 67911866 | 67915489 | |
| 56439 | 67979962 | 67982471 | 110066 |
| 56440 | 68024782 | 68026179 | |
| 56441 | 68036877 | 68044996 | 171067 |
| 56442 | 68064220 | 68067056 | |
| 56443 | 68069987 | 68070992 | |
| 56444 | 68079671 | 68083960 | 162337 |
| 56445 | 68088699 | 68090217 | 162337 |
| 56446 | 68093966 | 68094771 | 162337 |
| 56447 | 68111338 | 68111963 | 162337 |
| 56448 | 68121587 | 68122486 | 162337 |
| 56449 | 68144686 | 68145577 | 162337 |
| 56450 | 68153675 | 68154662 | 162337 |
| 56451 | 68154862 | 68155756 | 162337 |
| 56452 | 68223119 | 68223971 | |
| 56453 | 68227241 | 68229723 | 110075 |
| 56454 | 68236275 | 68237059 | 110075 |
| 56455 | 68265178 | 68265630 | 110075 |
| 56456 | 68309452 | 68310751 | 110075 |
| 56457 | 68313871 | 68314447 | 110075 |
| 56458 | 68518414 | 68519226 | 132749 |
| 56459 | 68584657 | 68585487 | 110090 |
| 56460 | 68605774 | 68607422 | 110090 |
| 56461 | 68607433 | 68609493 | 110090 |
| 56462 | 68610687 | 68611874 | 110090 |
| 56463 | 68612744 | 68613487 | |
| 56464 | 68624352 | 68626004 | |
| 56465 | 68645094 | 68645996 | |
| 56466 | 68670591 | 68672507 | 132740, 197345 |
| 56467 | 68768204 | 68768639 | 261625 |
| 56468 | 68774958 | 68776652 | 172935 |
| 56469 | 68815917 | 68817867 | 162341 |
| 56470 | 68822292 | 68823464 | 162341 |
| 56471 | 68882336 | 68882955 | |
| 56472 | 68890045 | 68891486 | |
| 56473 | 68898875 | 68899772 | |
| 56474 | 68933770 | 68934604 | 261070 |
| 56475 | 69041938 | 69042601 | |
| 56476 | 69063984 | 69067010 | 172927 |
| 56477 | 69067035 | 69070469 | 172927 |
| 56478 | 69073206 | 69074080 | 172927 |
| 56479 | 69165382 | 69169442 | 172927 |
| 56480 | 69171504 | 69172199 | 172927 |
| 56481 | 69174288 | 69176400 | 172927 |
| 56482 | 69242009 | 69242675 | 255980 |
| 56483 | 69245234 | 69245847 | |
| 56484 | 69251669 | 69252592 | |
| 56485 | 69284905 | 69285619 | |
| 56486 | 69294009 | 69296284 | 255774 |
| 56487 | 69299081 | 69301125 | |
| 56488 | 69311275 | 69312574 | |
| 56489 | 69355554 | 69357155 | |
| 56490 | 69451179 | 69458744 | 110092 |
| 56491 | 69469238 | 69470155 | 110092, 149716 |
| 56492 | 69489132 | 69490529 | 149716 |
| 56493 | 69502625 | 69505317 | |
| 56494 | 69515244 | 69516129 | 162344 |
| 56495 | 69681622 | 69684568 | |
| 56496 | 69705723 | 69708289 | |
| 56497 | 69714820 | 69715799 | |
| 56498 | 69720421 | 69721662 | |
| 56499 | 69793819 | 69797473 | |
| 56500 | 69814479 | 69817801 | |
| 56501 | 69824887 | 69826475 | |
| 56502 | 69831415 | 69833254 | 255191 |
| 56503 | 69845656 | 69846429 | 255191 |
| 56504 | 69875312 | 69876356 | |
| 56505 | 69880765 | 69881491 | |
| 56506 | 69923951 | 69925323 | 131620 |
| 56507 | 69926857 | 69927353 | 131620 |
| 56508 | 69932046 | 69932938 | 131620 |
| 56509 | 69973992 | 69975712 | 255143, 131620 |
| 56510 | 70005619 | 70006489 | 131620 |
| 56511 | 70048931 | 70051253 | 168040 |
| 56512 | 70058270 | 70059076 | |
| 56513 | 70066254 | 70066617 | |
| 56514 | 70115866 | 70117889 | 131626 |
| 56515 | 70172324 | 70173002 | 131626, 254604, 255539 |
| 56516 | 70243743 | 70246318 | 085733, 246889 |
| 56517 | 70327662 | 70329626 | 162105 |
| 56518 | 70502419 | 70502886 | 162105 |
| 56519 | 70890677 | 70891507 | 162105 |
| 56520 | 70913277 | 70914231 | 162105 |
| 56521 | 70918335 | 70919163 | 162105 |
| 56522 | 70952005 | 70953238 | 162105 |
| 56523 | 70961452 | 70965364 | 162105 |
| 56524 | 70997916 | 70999001 | |
| 56525 | 71009442 | 71011185 | |
| 56526 | 71017314 | 71017829 | |
| 56527 | 71143698 | 71145336 | 172893 |
| 56528 | 71158118 | 71160683 | 254682, 172893 |
| 56529 | 71163016 | 71165144 | 172890, 172893, 254682 |
| 56530 | 71200254 | 71200854 | 172890 |
| 56531 | 71236219 | 71238243 | |
| 56532 | 71638737 | 71641102 | 137522, 254469 |
| 56533 | 71655117 | 71660566 | 137522 |
| 56534 | 71723705 | 71724485 | 137497 |
| 56535 | 71751219 | 71753577 | 137497 |
| 56536 | 71780129 | 71781832 | 137497 |
| 56537 | 71790955 | 71792640 | 184154, 137497 |
| 56538 | 71802137 | 71802748 | 149357, 184154 |
| 56539 | 71813118 | 71815290 | 238768, 149357, 184154 |
| 56540 | 71934002 | 71936725 | 165458 |
| 56541 | 72144587 | 72145873 | 162129 |
| 56542 | 72186381 | 72187056 | |
| 56543 | 72353373 | 72355596 | 255808, 186642 |
| 56544 | 72431812 | 72434286 | 186635 |
| 56545 | 72459697 | 72460674 | 186635 |
| 56546 | 72462852 | 72464289 | 186635 |
| 56547 | 72477254 | 72477770 | 186635, 214530 |
| 56548 | 72486861 | 72489728 | 186635, 214530 |
| 56549 | 72489841 | 72493780 | 186635, 214530 |
| 56550 | 72498204 | 72499006 | 186635, 214530 |
| 56551 | 72500456 | 72501217 | 186635, 214530 |
| 56552 | 72503496 | 72505480 | 186635, 214530 |
| 56553 | 72518196 | 72518966 | |
| 56554 | 72523826 | 72526239 | 168010 |
| 56555 | 72538970 | 72539803 | 168010 |
| 56556 | 72661975 | 72662918 | 137478 |
| 56557 | 72710504 | 72711246 | 137478 |
| 56558 | 72766721 | 72767230 | 137478 |
| 56559 | 72850683 | 72854962 | 137478 |
| 56560 | 72858585 | 72859839 | |
| 56561 | 72884672 | 72885390 | |
| 56562 | 72886609 | 72887192 | |
| 56563 | 72909436 | 72910182 | |
| 56564 | 72916567 | 72917231 | |
| 56565 | 72928224 | 72930245 | 175591 |
| 56566 | 72999729 | 73000963 | 171631 |
| 56567 | 73087085 | 73088557 | 054967 |
| 56568 | 73204864 | 73205786 | 054965 |
| 56569 | 73265739 | 73266598 | 054965 |
| 56570 | 73308430 | 73310318 | 054965 |
| 56571 | 73470674 | 73473139 | 256034, 175582 |
| 56572 | 73489617 | 73491881 | |
| 56573 | 73497933 | 73499904 | 175581 |
| 56574 | 73529961 | 73530503 | 175581 |
| 56575 | 73576976 | 73578733 | |
| 56576 | 73586678 | 73588842 | 175575, 181924 |
| 56577 | 73618224 | 73618743 | 175575 |
| 56578 | 73625224 | 73625831 | 175575 |
| 56579 | 73729331 | 73730773 | 168014 |
| 56580 | 73795855 | 73796511 | 168014 |
| 56581 | 73849552 | 73850479 | 168014 |
| 56582 | 73881320 | 73883533 | 214517, 168014 |
| 56583 | 73988341 | 73991214 | 149380 |
| 56584 | 74108689 | 74110302 | 264402, 165434, 254631 |
| 56585 | 74129171 | 74130084 | 254631 |
| 56586 | 74204184 | 74205045 | 077514, 254837, 175536 |
| 56587 | 74303275 | 74304409 | 077514 |
| 56588 | 74320858 | 74321278 | 077514 |
| 56589 | 74373615 | 74374745 | 077514 |
| 56590 | 74382418 | 74385477 | |
| 56591 | 74407316 | 74409781 | 185162, 054938 |
| 56592 | 74459490 | 74461172 | 166439 |
| 56593 | 74465767 | 74466567 | 166439 |
| 56594 | 74606819 | 74607798 | 166435, 241170 |
| 56595 | 74659400 | 74661534 | 118363, 166435 |
| 56596 | 74695103 | 74695635 | |
| 56597 | 74699323 | 74700914 | 162139 |
| 56598 | 74811178 | 74811883 | 137491, 255395 |
| 56599 | 74862533 | 74863903 | 137491 |
| 56600 | 74865682 | 74867777 | 137491 |
| 56601 | 74869631 | 74872173 | 137491 |
| 56602 | 74872296 | 74874390 | 137491 |
| 56603 | 74875078 | 74876475 | 137491 |
| 56604 | 74879258 | 74880150 | 137491 |
| 56605 | 74901577 | 74902960 | 137491 |
| 56606 | 74905941 | 74907213 | 137491 |
| 56607 | 74914855 | 74916870 | 137491 |
| 56608 | 74952227 | 74953221 | 261594 |
| 56609 | 75053811 | 75054508 | 137486 |
| 56610 | 75061997 | 75063160 | 137486 |
| 56611 | 75083237 | 75083817 | |
| 56612 | 75110318 | 75111900 | 206941, 149273 |
| 56613 | 75133046 | 75134288 | 149243, 149273 |
| 56614 | 75193815 | 75194501 | 158555 |
| 56615 | 75236211 | 75237473 | 158555 |
| 56616 | 75272587 | 75275067 | 149257 |
| 56617 | 75293705 | 75295145 | 255326 |
| 56618 | 75298758 | 75299826 | 171533, 255326 |
| 56619 | 75408935 | 75409777 | |
| 56620 | 75429247 | 75431582 | 166391 |
| 56621 | 75432541 | 75434011 | 166391 |
| 56622 | 75434151 | 75435323 | 166391 |
| 56623 | 75443944 | 75446849 | 166391 |
| 56624 | 75448151 | 75449149 | |
| 56625 | 75454638 | 75455864 | |
| 56626 | 75473822 | 75474439 | 247867, 062282 |
| 56627 | 75478716 | 75481074 | 247867, 062282 |
| 56628 | 75482644 | 75483509 | 062282 |
| 56629 | 75483818 | 75485664 | 062282 |
| 56630 | 75493210 | 75495647 | 062282 |
| 56631 | 75504034 | 75504600 | 062282 |
| 56632 | 75506335 | 75507522 | 062282 |
| 56633 | 75523770 | 75525034 | 255507 |
| 56634 | 75525040 | 75527310 | 198382, 255507 |
| 56635 | 75677825 | 75678477 | 198382 |
| 56636 | 75919355 | 75922219 | 255362, 085741 |
| 56637 | 75938751 | 75940224 | |
| 56638 | 75946251 | 75947682 | |
| 56639 | 75976572 | 75978436 | |
| 56640 | 75979993 | 75981483 | |
| 56641 | 76090960 | 76092851 | 179240, 137492 |
| 56642 | 76096875 | 76097530 | 179240 |
| 56643 | 76154769 | 76156915 | 158636, 255135 |
| 56644 | 76476715 | 76485913 | 254632 |
| 56645 | 76491911 | 76500793 | 182704, 255100 |
| 56646 | 76507182 | 76508823 | 182704 |
| 56647 | 76509974 | 76511109 | |
| 56648 | 76511200 | 76513306 | 261578 |
| 56649 | 76571409 | 76572782 | 078124 |
| 56650 | 76771909 | 76772838 | |
| 56651 | 76776831 | 76778457 | 149260 |
| 56652 | 76797311 | 76798697 | 149260 |
| 56653 | 76806475 | 76807913 | 149260 |
| 56654 | 76816927 | 76820023 | 149260 |
| 56655 | 76837983 | 76840237 | 137474 |
| 56656 | 76840807 | 76842805 | 137474 |
| 56657 | 76847197 | 76848505 | 137474 |
| 56658 | 76855583 | 76856578 | 137474 |
| 56659 | 76889860 | 76890583 | 137474 |
| 56660 | 76953918 | 76955318 | 178795 |
| 56661 | 76963670 | 76965040 | 178795 |
| 56662 | 77170112 | 77170907 | 149269 |
| 56663 | 77183879 | 77186233 | 268635, 149269 |
| 56664 | 77270329 | 77272751 | 074201 |
| 56665 | 77299760 | 77302277 | 178301, 074201 |
| 56666 | 77312233 | 77314830 | 178301, 074201 |
| 56667 | 77341752 | 77342498 | 074201 |
| 56668 | 77347838 | 77349647 | 074201 |
| 56669 | 77530458 | 77532994 | 087884, 048649 |
| 56670 | 77705422 | 77706473 | 149262 |
| 56671 | 77749586 | 77751724 | 259112, 151364 |
| 56672 | 77756654 | 77757705 | 151364, 259112 |
| 56673 | 77768216 | 77769172 | 259112 |
| 56674 | 77771810 | 77773364 | 259112 |
| 56675 | 77774288 | 77775706 | 151365, 259112 |
| 56676 | 77789744 | 77791649 | 259112, 151366 |
| 56677 | 77801600 | 77802151 | |
| 56678 | 77849705 | 77851556 | 246174, 159063 |
| 56679 | 77867186 | 77868217 | 246174 |
| 56680 | 77879064 | 77882543 | 188997, 246174 |
| 56681 | 77890722 | 77891146 | 188997 |
| 56682 | 77896007 | 77897313 | 188997 |
| 56683 | 77898019 | 77900572 | 118369, 188997 |
| 56684 | 77988333 | 77989137 | 033327 |
| 56685 | 78000772 | 78002251 | 033327 |
| 56686 | 78022621 | 78023559 | 033327 |
| 56687 | 78117418 | 78118287 | 254420, 033327 |
| 56688 | 78127193 | 78130160 | 033327, 254420 |
| 56689 | 78130266 | 78130922 | 254420 |
| 56690 | 78260957 | 78261616 | 255084, 137513 |
| 56691 | 78285147 | 78286105 | 137513 |
| 56692 | 78618482 | 78619244 | 149256 |
| 56693 | 78717745 | 78718372 | 149256 |
| 56694 | 78755697 | 78757367 | 149256 |
| 56695 | 78760517 | 78761491 | 149256 |
| 56696 | 78797918 | 78799049 | 149256 |
| 56697 | 79199288 | 79199718 | |
| 56698 | 82610761 | 82612851 | 165490, 137509 |
| 56699 | 82762716 | 82763293 | 137502 |
| 56700 | 82781385 | 82784495 | 246067, 255503, 137502 |
| 56701 | 82803224 | 82804703 | 246067, 255503 |
| 56702 | 82866726 | 82870185 | 165494 |
| 56703 | 82903290 | 82905575 | 137494, 247137 |
| 56704 | 82968873 | 82969302 | 137494 |
| 56705 | 82995312 | 82997958 | 255234, 137500 |
| 56706 | 83219677 | 83220526 | 150672 |
| 56707 | 83359759 | 83361277 | 255311, 150672 |
| 56708 | 83371858 | 83374327 | 255311, 150672 |
| 56709 | 83402411 | 83402880 | 255311, 150672 |
| 56710 | 83412786 | 83413937 | 255311, 150672 |
| 56711 | 83421574 | 83422844 | 255311, 150672 |
| 56712 | 83424626 | 83425404 | 255311, 150672 |
| 56713 | 83428444 | 83428948 | 255311, 150672 |
| 56714 | 83434892 | 83436016 | 255311, 150672 |
| 56715 | 83562759 | 83565879 | 150672 |
| 56716 | 83575113 | 83577721 | 150672 |
| 56717 | 83632651 | 83633316 | 150672 |
| 56718 | 83767582 | 83768111 | 150672 |
| 56719 | 83955418 | 83955969 | 150672 |
| 56720 | 83960311 | 83962718 | 150672 |
| 56721 | 84269085 | 84270021 | 150672 |
| 56722 | 85194828 | 85195402 | 150672 |
| 56723 | 85337468 | 85340605 | 171204, 150672 |
| 56724 | 85358274 | 85359951 | 171202 |
| 56725 | 85374176 | 85377234 | 137504 |
| 56726 | 85434139 | 85434565 | 137501 |
| 56727 | 85555229 | 85556382 | |
| 56728 | 85565471 | 85566489 | 150676, 215504 |
| 56729 | 85658903 | 85659582 | |
| 56730 | 85758214 | 85758689 | 073921 |
| 56731 | 85776893 | 85781894 | 073921 |
| 56732 | 85799732 | 85800435 | |
| 56733 | 85812131 | 85813650 | |
| 56734 | 85826549 | 85829580 | |
| 56735 | 85884428 | 85888575 | |
| 56736 | 85955019 | 85957315 | 074266 |
| 56737 | 86002401 | 86003444 | |
| 56738 | 86013010 | 86014428 | 149196 |
| 56739 | 86166992 | 86172289 | 254733, 151376 |
| 56740 | 86194470 | 86197044 | 254733, 151376 |
| 56741 | 86210580 | 86212094 | 254733, 151376 |
| 56742 | 86217892 | 86223244 | 254733, 151376 |
| 56743 | 86233338 | 86236808 | 254733, 151376 |
| 56744 | 86262005 | 86264069 | 254733, 151376 |
| 56745 | 86288728 | 86290872 | 254733, 151376 |
| 56746 | 86302737 | 86306261 | 254733, 151376 |
| 56747 | 86317516 | 86319881 | 254733, 151376 |
| 56748 | 86381859 | 86384638 | 151376 |
| 56749 | 86406472 | 86407844 | |
| 56750 | 86428830 | 86429619 | |
| 56751 | 86430159 | 86433412 | |
| 56752 | 86436282 | 86437942 | |
| 56753 | 86448904 | 86455713 | 255250 |
| 56754 | 86510770 | 86514191 | 150687 |
| 56755 | 86522277 | 86523904 | 150687 |
| 56756 | 86650527 | 86653673 | 150687 |
| 56757 | 86665403 | 86667956 | 246523, 174804 |
| 56758 | 86694111 | 86694577 | 246523 |
| 56759 | 86747842 | 86749986 | 166575 |
| 56760 | 86792195 | 86793151 | 166575 |
| 56761 | 86808403 | 86809438 | 166575 |
| 56762 | 86884263 | 86885139 | 166575 |
| 56763 | 87063666 | 87064470 | |
| 56764 | 87130247 | 87130999 | |
| 56765 | 87148551 | 87149637 | |
| 56766 | 87835686 | 87839197 | 255241 |
| 56767 | 88070122 | 88071808 | 109861 |
| 56768 | 88547175 | 88547699 | 168959 |
| 56769 | 88560091 | 88561297 | 168959 |
| 56770 | 89955191 | 89957119 | 110172 |
| 56771 | 90019710 | 90020220 | 261645 |
| 56772 | 90455841 | 90456505 | 261645 |
| 56773 | 91129405 | 91130505 | |
| 56774 | 91406614 | 91407560 | |
| 56775 | 91655125 | 91655678 | |
| 56776 | 91679441 | 91680035 | |
| 56777 | 91699532 | 91700681 | |
| 56778 | 91705890 | 91706513 | |
| 56779 | 91714226 | 91718491 | |
| 56780 | 91768544 | 91769565 | |
| 56781 | 91874135 | 91874744 | |
| 56782 | 91999495 | 92000769 | |
| 56783 | 92001012 | 92003531 | |
| 56784 | 92089857 | 92092393 | 165323 |
| 56785 | 92757896 | 92760520 | |
| 56786 | 92767081 | 92768189 | |
| 56787 | 92787116 | 92789327 | |
| 56788 | 92930126 | 92931601 | 180773 |
| 56789 | 93220140 | 93220798 | 166002 |
| 56790 | 93225609 | 93226951 | 166002 |
| 56791 | 93237172 | 93238216 | 166002 |
| 56792 | 93258056 | 93259233 | 166002 |
| 56793 | 93259238 | 93260335 | 166002 |
| 56794 | 93270140 | 93271880 | 166002 |
| 56795 | 93274921 | 93277388 | 166002 |
| 56796 | 93285786 | 93286794 | |
| 56797 | 93300496 | 93301518 | |
| 56798 | 93303703 | 93304785 | |
| 56799 | 93394451 | 93395875 | 166004 |
| 56800 | 93472456 | 93476249 | 182919, 166012 |
| 56801 | 93517213 | 93518440 | 042429, 166012 |
| 56802 | 93849311 | 93849997 | |
| 56803 | 93861041 | 93865123 | 110218 |
| 56804 | 93867537 | 93869523 | 110218 |
| 56805 | 93883470 | 93887457 | 110218 |
| 56806 | 93887477 | 93888090 | 110218 |
| 56807 | 93926443 | 93927615 | |
| 56808 | 93929370 | 93930563 | |
| 56809 | 93934084 | 93934705 | |
| 56810 | 94181651 | 94183220 | 020922 |
| 56811 | 94226214 | 94227917 | 168876, 020922 |
| 56812 | 94276426 | 94278373 | 196371, 134627 |
| 56813 | 94316826 | 94317916 | 255929, 134627 |
| 56814 | 94335390 | 94336512 | 255929, 134627 |
| 56815 | 94527123 | 94530224 | 166025 |
| 56816 | 94546585 | 94548178 | 166025 |
| 56817 | 94565733 | 94566407 | 166025 |
| 56818 | 94578615 | 94579300 | 166025 |
| 56819 | 94799523 | 94802133 | 180771, 271885 |
| 56820 | 94822409 | 94823800 | 149218 |
| 56821 | 94883318 | 94885482 | 245552 |
| 56822 | 94962977 | 94966346 | 149212, 245552 |
| 56823 | 94999739 | 95000525 | |
| 56824 | 95002308 | 95003144 | |
| 56825 | 95015187 | 95016203 | |
| 56826 | 95070566 | 95072975 | |
| 56827 | 95091564 | 95093124 | |
| 56828 | 95097263 | 95098317 | |
| 56829 | 95111287 | 95111718 | |
| 56830 | 95255958 | 95256588 | |
| 56831 | 95374584 | 95375120 | |
| 56832 | 95413630 | 95414379 | |
| 56833 | 95521971 | 95525089 | 166037, 077458 |
| 56834 | 95528462 | 95529484 | 166037 |
| 56835 | 95656462 | 95658622 | 087053 |
| 56836 | 95831934 | 95832328 | 184384 |
| 56837 | 96075441 | 96076918 | 184384 |
| 56838 | 96121937 | 96124079 | 183340, 149231 |
| 56839 | 98706060 | 98706470 | |
| 56840 | 99127586 | 99128475 | 149972 |
| 56841 | 100456374 | 100456905 | |
| 56842 | 100501357 | 100502609 | |
| 56843 | 100503402 | 100504435 | |
| 56844 | 100538069 | 100538635 | |
| 56845 | 100557199 | 100559337 | 165895, 248027 |
| 56846 | 100590242 | 100591040 | 165895 |
| 56847 | 100610235 | 100610826 | 165895 |
| 56848 | 100645261 | 100648875 | 165895 |
| 56849 | 100655984 | 100656983 | 165895 |
| 56850 | 100659488 | 100661187 | 165895 |
| 56851 | 100701883 | 100704584 | 165895 |
| 56852 | 100719484 | 100720895 | 165895 |
| 56853 | 100721921 | 100723056 | 165895 |
| 56854 | 100746534 | 100748543 | 165895 |
| 56855 | 100773276 | 100774535 | 165895 |
| 56856 | 100851936 | 100852500 | 165895 |
| 56857 | 101124282 | 101125520 | |
| 56858 | 101736644 | 101739155 | 137672 |
| 56859 | 101877472 | 101879095 | |
| 56860 | 101980223 | 101983864 | 137693, 260008 |
| 56861 | 101986001 | 101986709 | 137693 |
| 56862 | 102041818 | 102043651 | 137693 |
| 56863 | 102048374 | 102050146 | 137693 |
| 56864 | 102112785 | 102113248 | |
| 56865 | 102138824 | 102140380 | |
| 56866 | 102150232 | 102150881 | |
| 56867 | 102176190 | 102177929 | 270868 |
| 56868 | 102183010 | 102185335 | 212466 |
| 56869 | 102186643 | 102189762 | 023445 |
| 56870 | 102215806 | 102219310 | 110330 |
| 56871 | 102321802 | 102324518 | 255337, 152558 |
| 56872 | 102327020 | 102327617 | 152558, 255337 |
| 56873 | 102329134 | 102329742 | 152558, 255337 |
| 56874 | 102331578 | 102334038 | 255337, 152558 |
| 56875 | 102339481 | 102340418 | 255482, 152558 |
| 56876 | 102346368 | 102348325 | 255482 |
| 56877 | 102355463 | 102357357 | 255482 |
| 56878 | 102364229 | 102366562 | 255482 |
| 56879 | 102423146 | 102423610 | |
| 56880 | 102469158 | 102471726 | 137674 |
| 56881 | 102473687 | 102474254 | 137674 |
| 56882 | 102511182 | 102512418 | |
| 56883 | 102702056 | 102703200 | 255282 |
| 56884 | 102800543 | 102801176 | |
| 56885 | 102914585 | 102915275 | |
| 56886 | 102961978 | 102963764 | 137692 |
| 56887 | 102979655 | 102980582 | 187240 |
| 56888 | 104827163 | 104827755 | 196954 |
| 56889 | 104838312 | 104840402 | 196954 |
| 56890 | 104875387 | 104876305 | 137757 |
| 56891 | 104905573 | 104906464 | 137752 |
| 56892 | 104917814 | 104918492 | 137752, 204397 |
| 56893 | 105892016 | 105893689 | 170903 |
| 56894 | 105946848 | 105949302 | 182359, 149313 |
| 56895 | 107028061 | 107028983 | |
| 56896 | 107067311 | 107068321 | |
| 56897 | 107327689 | 107329072 | 152404 |
| 56898 | 107420005 | 107420912 | 137760 |
| 56899 | 107435607 | 107437062 | 137760 |
| 56900 | 107480179 | 107481204 | 110675 |
| 56901 | 107662238 | 107662885 | 110660 |
| 56902 | 107879293 | 107880932 | 166266 |
| 56903 | 107940653 | 107941110 | 166266 |
| 56904 | 107985690 | 107986859 | 255467 |
| 56905 | 107991296 | 107995011 | 075239, 255467 |
| 56906 | 107995146 | 107996168 | 075239 |
| 56907 | 108092277 | 108095064 | 149311, 149308 |
| 56908 | 108104298 | 108104788 | 149311 |
| 56909 | 108337114 | 108338808 | 166323 |
| 56910 | 108345438 | 108346422 | 178202 |
| 56911 | 108367675 | 108369782 | 178202 |
| 56912 | 108382359 | 108383154 | 110723 |
| 56913 | 108438164 | 108439532 | 110723 |
| 56914 | 108463141 | 108464951 | 110723 |
| 56915 | 108517785 | 108519730 | |
| 56916 | 108535244 | 108536622 | 178105 |
| 56917 | 108646016 | 108646436 | 178105 |
| 56918 | 108956314 | 108957185 | |
| 56919 | 109816623 | 109818048 | |
| 56920 | 109962583 | 109965260 | 149289 |
| 56921 | 109996319 | 109996937 | 149289 |
| 56922 | 110066172 | 110069103 | 137710 |
| 56923 | 110122469 | 110123131 | 137710 |
| 56924 | 110165828 | 110169592 | 137710 |
| 56925 | 110215652 | 110217510 | |
| 56926 | 110225536 | 110229537 | 254416 |
| 56927 | 110233190 | 110234614 | 254416 |
| 56928 | 110243274 | 110244580 | 254416 |
| 56929 | 110247221 | 110248748 | 254416 |
| 56930 | 110300023 | 110302777 | 137714 |
| 56931 | 110582203 | 110584329 | 137727 |
| 56932 | 110633816 | 110635019 | |
| 56933 | 110860390 | 110860847 | |
| 56934 | 111101159 | 111101959 | |
| 56935 | 111169368 | 111170943 | 214290, 196167 |
| 56936 | 111172105 | 111172974 | 196167, 214290 |
| 56937 | 111174049 | 111174672 | 196167, 214290 |
| 56938 | 111178568 | 111179601 | 214290 |
| 56939 | 111191393 | 111192189 | |
| 56940 | 111205081 | 111206498 | |
| 56941 | 111229965 | 111232411 | 110777 |
| 56942 | 111232494 | 111232887 | 110777 |
| 56943 | 111233426 | 111234170 | 110777 |
| 56944 | 111472581 | 111476271 | 170145 |
| 56945 | 111477198 | 111478329 | 170145 |
| 56946 | 111494101 | 111494859 | 170145 |
| 56947 | 111503858 | 111504489 | 170145 |
| 56948 | 111547030 | 111549207 | 170145 |
| 56949 | 111558378 | 111559777 | 170145 |
| 56950 | 111591727 | 111592415 | 170145 |
| 56951 | 111596625 | 111599057 | 137713, 170145 |
| 56952 | 111599656 | 111610690 | 170145, 137713 |
| 56953 | 111616048 | 111616814 | 137713 |
| 56954 | 111635547 | 111638145 | 137713 |
| 56955 | 111645538 | 111646546 | 254990 |
| 56956 | 111647431 | 111649499 | 254990 |
| 56957 | 111656510 | 111657331 | 258529, 086848 |
| 56958 | 111658652 | 111659545 | 086848, 258529 |
| 56959 | 111660773 | 111661433 | 086848, 258529 |
| 56960 | 111664430 | 111668951 | 086848, 258529 |
| 56961 | 111669972 | 111672727 | 086848, 258529 |
| 56962 | 111677069 | 111677576 | 086848, 258529 |
| 56963 | 111694178 | 111694904 | 254450, 086848, 258529 |
| 56964 | 111710596 | 111711559 | 086848, 258529 |
| 56965 | 111741391 | 111742846 | 086848, 258529 |
| 56966 | 111748792 | 111751011 | 137720, 258529, 255561 |
| 56967 | 111782287 | 111783251 | 170276, 109846 |
| 56968 | 111789082 | 111790187 | 170276, 149300, 109846, |
| | | | 254445 |
| 56969 | 111796803 | 111797978 | 150764, 149300, 254445 |
| 56970 | 111801156 | 111801506 | 150764 |
| 56971 | 111805808 | 111806517 | 150764 |
| 56972 | 111807250 | 111808483 | 150764 |
| 56973 | 111831036 | 111833089 | 150764 |
| 56974 | 111847539 | 111848701 | 150764 |
| 56975 | 111852635 | 111854509 | 150764 |
| 56976 | 111858663 | 111860532 | 150764 |
| 56977 | 111867154 | 111868531 | 150764 |
| 56978 | 111883366 | 111884067 | 150764 |
| 56979 | 111886510 | 111890195 | 255334, 150764 |
| 56980 | 111894798 | 111897248 | 150768 |
| 56981 | 111944461 | 111946027 | 150776, 150773 |
| 56982 | 111956312 | 111958483 | 255292, 204370, 150779 |
| 56983 | 111971185 | 111971751 | 255292, 204370 |
| 56984 | 112026783 | 112032298 | 255292, 150782 |
| 56985 | 112032378 | 112035763 | 150782, 255292 |
| 56986 | 112036380 | 112038263 | 150783, 254638, 255292 |
| 56987 | 112045479 | 112048791 | 197580, 272790, 255292 |
| 56988 | 112084375 | 112085226 | 197580 |
| 56989 | 112087040 | 112088074 | 197580 |
| 56990 | 112091352 | 112093132 | 197580 |
| 56991 | 112096336 | 112098287 | 150787 |
| 56992 | 112137803 | 112138371 | 213234, 150787 |
| 56993 | 112139450 | 112140516 | 150787 |
| 56994 | 112150456 | 112151895 | 250303 |
| 56995 | 112160142 | 112162148 | 250303 |
| 56996 | 112172802 | 112173581 | 250303 |
| 56997 | 112191390 | 112193604 | 250303 |
| 56998 | 112234169 | 112234747 | |
| 56999 | 112434972 | 112435867 | 254968 |
| 57000 | 113184794 | 113186045 | 149292, 255129 |
| 57001 | 113643588 | 113645058 | 086827 |
| 57002 | 113648524 | 113649449 | |
| 57003 | 113652304 | 113653418 | |
| 57004 | 113745375 | 113747155 | 048028 |
| 57005 | 113888422 | 113888854 | |
| 57006 | 113906683 | 113908218 | |
| 57007 | 113911317 | 113911968 | |
| 57008 | 113928168 | 113933081 | 109906 |
| 57009 | 113934720 | 113935714 | 109906 |
| 57010 | 113942385 | 113943347 | 109906 |
| 57011 | 113949036 | 113949772 | 109906 |
| 57012 | 113950010 | 113950625 | 109906 |
| 57013 | 113950892 | 113952732 | 109906 |
| 57014 | 113952854 | 113954125 | 109906 |
| 57015 | 113956687 | 113957238 | 109906 |
| 57016 | 113969307 | 113970682 | 109906 |
| 57017 | 113981068 | 113982969 | 109906 |
| 57018 | 113992348 | 113993310 | 109906 |
| 57019 | 114005453 | 114006720 | 109906 |
| 57020 | 114017985 | 114018978 | 109906 |
| 57021 | 114019549 | 114022960 | 109906 |
| 57022 | 114023322 | 114026895 | 109906 |
| 57023 | 114028593 | 114039972 | 109906 |
| 57024 | 114040081 | 114052088 | 109906 |
| 57025 | 114066882 | 114070002 | 109906 |
| 57026 | 114079920 | 114081079 | 109906 |
| 57027 | 114127924 | 114129490 | 166741, 256947 |
| 57028 | 114136121 | 114136834 | 256947, 166741 |
| 57029 | 114148195 | 114149502 | 256947, 166741 |
| 57030 | 114161509 | 114164438 | 256947, 166741 |
| 57031 | 114165058 | 114169688 | 256947, 166741 |
| 57032 | 114174273 | 114175351 | 256947, 166741 |
| 57033 | 114177577 | 114180482 | 256947, 166741 |
| 57034 | 114186272 | 114187287 | 256947 |
| 57035 | 114216295 | 114216959 | 256947 |
| 57036 | 114269766 | 114272667 | 076053, 255663, 180425 |
| 57037 | 114309902 | 114311325 | 076043, 255663 |
| 57038 | 115096395 | 115098095 | 182985 |
| 57039 | 115150830 | 115151580 | 182985 |
| 57040 | 115247890 | 115249650 | 255580, 182985 |
| 57041 | 115309997 | 115310490 | 182985 |
| 57042 | 115341940 | 115343070 | 182985 |
| 57043 | 115358940 | 115360731 | 182985 |
| 57044 | 115372598 | 115376539 | 182985 |
| 57045 | 115393837 | 115395219 | |
| 57046 | 115419121 | 115420879 | |
| 57047 | 115462790 | 115463717 | 255689 |
| 57048 | 115481556 | 115482390 | |
| 57049 | 115482876 | 115483755 | |
| 57050 | 115493625 | 115494219 | |
| 57051 | 115497069 | 115497757 | |
| 57052 | 115526795 | 115528271 | |
| 57053 | 115529518 | 115530713 | 256717 |
| 57054 | 115787332 | 115787851 | |
| 57055 | 115789432 | 115791268 | |
| 57056 | 115791548 | 115792646 | |
| 57057 | 115834624 | 115835242 | |
| 57058 | 116282763 | 116283972 | |
| 57059 | 116290787 | 116291617 | |
| 57060 | 116342273 | 116343019 | |
| 57061 | 116433751 | 116434951 | |
| 57062 | 116447024 | 116448373 | |
| 57063 | 116505987 | 116506453 | |
| 57064 | 116531644 | 116532443 | |
| 57065 | 116558102 | 116558710 | |
| 57066 | 116642277 | 116644853 | 226645, 137656 |
| 57067 | 116657761 | 116659639 | 109917 |
| 57068 | 116661838 | 116664084 | 110243 |
| 57069 | 116678100 | 116681582 | |
| 57070 | 116683324 | 116685801 | 236267 |
| 57071 | 116695684 | 116698971 | |
| 57072 | 116699023 | 116701866 | 110245 |
| 57073 | 116702757 | 116705255 | 110245 |
| 57074 | 116705688 | 116707284 | 235910, 118137 |
| 57075 | 116707334 | 116710443 | 118137, 235910 |
| 57076 | 116734801 | 116735801 | 160584 |
| 57077 | 116799702 | 116800763 | 160584 |
| 57078 | 116857084 | 116857935 | 160584 |
| 57079 | 116860493 | 116861419 | 160584 |
| 57080 | 116890312 | 116892861 | 160584 |
| 57081 | 116915597 | 116916385 | 160584 |
| 57082 | 116916629 | 116917124 | 160584 |
| 57083 | 116941961 | 116942795 | 160584 |
| 57084 | 116943103 | 116944347 | 160584 |
| 57085 | 116947611 | 116949066 | 160584 |
| 57086 | 116966516 | 116967742 | 160584 |
| 57087 | 116967776 | 116970617 | 224077, 160584 |
| 57088 | 117001559 | 117002165 | |
| 57089 | 117014127 | 117016349 | 168092, 254678 |
| 57090 | 117049195 | 117050821 | 149577 |
| 57091 | 117101693 | 117104348 | 167257, 160613 |
| 57092 | 117169797 | 117171515 | 186318 |
| 57093 | 117186146 | 117187713 | 250699, 186318, 110274 |
| 57094 | 117198149 | 117199522 | 250699, 110274 |
| 57095 | 117687639 | 117689940 | 254844, 177103, 137731 |
| 57096 | 118015814 | 118016823 | 177098 |
| 57097 | 118045111 | 118046487 | 149575 |
| 57098 | 118064465 | 118066558 | 160593 |
| 57099 | 118073042 | 118073875 | 160593 |
| 57100 | 118082761 | 118083623 | 160593 |
| 57101 | 118117328 | 118118445 | 160588 |
| 57102 | 118121786 | 118123565 | 160588 |
| 57103 | 118134223 | 118135385 | 149573 |
| 57104 | 118185584 | 118186360 | 198851 |
| 57105 | 118192112 | 118192665 | |
| 57106 | 118229694 | 118231319 | 110344 |
| 57107 | 118271116 | 118273357 | 167283, 254873 |
| 57108 | 118304889 | 118306235 | 255384 |
| 57109 | 118306282 | 118308325 | 118058 |
| 57110 | 118397136 | 118398334 | 172425, 118058, 255435 |
| 57111 | 118400869 | 118402217 | 149582, 255435, 172425 |
| 57112 | 118404204 | 118405121 | 149582 |
| 57113 | 118436405 | 118437069 | 118096 |
| 57114 | 118442048 | 118444288 | 095139, 118096 |
| 57115 | 118478186 | 118479584 | 019144 |
| 57116 | 118540691 | 118541222 | 118094 |
| 57117 | 118549698 | 118550936 | 118094 |
| 57118 | 118560354 | 118561445 | 255239 |
| 57119 | 118612944 | 118613490 | 255422 |
| 57120 | 118618752 | 118619335 | 255422 |
| 57121 | 118628529 | 118629248 | 110367 |
| 57122 | 118660178 | 118663913 | 110367 |
| 57123 | 118691106 | 118691913 | |
| 57124 | 118709534 | 118710167 | |
| 57125 | 118712408 | 118713031 | |
| 57126 | 118740370 | 118742028 | |
| 57127 | 118779967 | 118785226 | 264211, 186174 |
| 57128 | 118786177 | 118797145 | 110375, 186174 |
| 57129 | 118797568 | 118801435 | 110375 |
| 57130 | 118888063 | 118891201 | 266398, 196655, 118181 |
| 57131 | 118891634 | 118892963 | 196655 |
| 57132 | 118892998 | 118893641 | 196655 |
| 57133 | 118898573 | 118901939 | 137700 |
| 57134 | 118927013 | 118928573 | 149428 |
| 57135 | 118955096 | 118956128 | 256269 |
| 57136 | 118964637 | 118966567 | 188486 |
| 57137 | 118971523 | 118973709 | 172375, 172269 |
| 57138 | 118977619 | 118979015 | 172269, 172375 |
| 57139 | 118991378 | 118992988 | 172273 |
| 57140 | 119039050 | 119040699 | 160703 |
| 57141 | 119076203 | 119077953 | 110395 |
| 57142 | 119186159 | 119186823 | 076706 |
| 57143 | 119204810 | 119206802 | 173456 |
| 57144 | 119210257 | 119211585 | 223953, 235718, 259159 |
| 57145 | 119231192 | 119233680 | 036672 |
| 57146 | 119233699 | 119236020 | 036672 |
| 57147 | 119238393 | 119242287 | 036672 |
| 57148 | 119246183 | 119247595 | 036672, 254740 |
| 57149 | 119251429 | 119252811 | 245248, 036672, 254740 |
| 57150 | 119351864 | 119352600 | 245248 |
| 57151 | 119404518 | 119405511 | |
| 57152 | 119486205 | 119486893 | 254561 |
| 57153 | 119569744 | 119570497 | 110400 |
| 57154 | 119598439 | 119601196 | 254854, 110400 |
| 57155 | 119630263 | 119631050 | |
| 57156 | 119635010 | 119635553 | |
| 57157 | 119777588 | 119778359 | |
| 57158 | 119995520 | 119996216 | 137699 |
| 57159 | 120039151 | 120040411 | 176984, 137699 |
| 57160 | 120053484 | 120054145 | 137699 |
| 57161 | 120054776 | 120058802 | 137699 |
| 57162 | 120063393 | 120064546 | |
| 57163 | 120078357 | 120084091 | 184232 |
| 57164 | 120086055 | 120087390 | 184232 |
| 57165 | 120165648 | 120166118 | 137709 |
| 57166 | 120170355 | 120171233 | 137709 |
| 57167 | 120195570 | 120196970 | 181264 |
| 57168 | 120201602 | 120202838 | 181264 |
| 57169 | 120205442 | 120209440 | 196914 |
| 57170 | 120262006 | 120262653 | 196914 |
| 57171 | 120268749 | 120269244 | 196914 |
| 57172 | 120270844 | 120272596 | 196914 |
| 57173 | 120302407 | 120303351 | 196914 |
| 57174 | 120312314 | 120313236 | 196914 |
| 57175 | 120352776 | 120353228 | 196914 |
| 57176 | 120418515 | 120419068 | 149403 |
| 57177 | 120479516 | 120479955 | 149403 |
| 57178 | 120482884 | 120484330 | 149403 |
| 57179 | 120545211 | 120545730 | 149403 |
| 57180 | 120797145 | 120797788 | 149403 |
| 57181 | 120823244 | 120824145 | 149403 |
| 57182 | 120856585 | 120857429 | 149403 |
| 57183 | 120894510 | 120895735 | 154114 |
| 57184 | 120902808 | 120903934 | 154114 |
| 57185 | 120906025 | 120906456 | 154114 |
| 57186 | 120962041 | 120963396 | |
| 57187 | 121101948 | 121103089 | |
| 57188 | 121162236 | 121164613 | 109929 |
| 57189 | 121227291 | 121229087 | |
| 57190 | 121318947 | 121320022 | 246790 |
| 57191 | 121321761 | 121325342 | 137642, 246790 |
| 57192 | 121331247 | 121332980 | 137642 |
| 57193 | 121333653 | 121336060 | 137642 |
| 57194 | 121336302 | 121339224 | 137642 |
| 57195 | 121340231 | 121343423 | 137642 |
| 57196 | 121346602 | 121347538 | 137642 |
| 57197 | 121423349 | 121426403 | 137642 |
| 57198 | 121435121 | 121436521 | 137642 |
| 57199 | 121445816 | 121447470 | 137642 |
| 57200 | 121456365 | 121458695 | 137642 |
| 57201 | 121465652 | 121467461 | 137642 |
| 57202 | 121525583 | 121527917 | |
| 57203 | 121802243 | 121803771 | |
| 57204 | 121806439 | 121808209 | |
| 57205 | 122007197 | 122008748 | |
| 57206 | 122010847 | 122012434 | |
| 57207 | 122032758 | 122033729 | 255090 |
| 57208 | 122057068 | 122058226 | 255090 |
| 57209 | 122058539 | 122061224 | 255090 |
| 57210 | 122077705 | 122079250 | 255090 |
| 57211 | 122079340 | 122082006 | 255090 |
| 57212 | 122238453 | 122239404 | 255090 |
| 57213 | 122253807 | 122254270 | 255090 |
| 57214 | 122310270 | 122312477 | |
| 57215 | 122451197 | 122452244 | |
| 57216 | 122512933 | 122514513 | |
| 57217 | 122526243 | 122527334 | 154127 |
| 57218 | 122931787 | 122935009 | 109971 |
| 57219 | 122954075 | 122954817 | 166250 |
| 57220 | 123611414 | 123613068 | 166261 |
| 57221 | 123669109 | 123669700 | |
| 57222 | 123985802 | 123986438 | 110002 |
| 57223 | 124272271 | 124272728 | |
| 57224 | 124491980 | 124493634 | 154144 |
| 57225 | 124542880 | 124544289 | 064199, 110013 |
| 57226 | 124616021 | 124617375 | 019102, 154146, 255045 |
| 57227 | 124639249 | 124640622 | 120458 |
| 57228 | 124669277 | 124671425 | 245498, 120458 |
| 57229 | 124706290 | 124706661 | |
| 57230 | 124745568 | 124747119 | 154134 |
| 57231 | 124752056 | 124753252 | |
| 57232 | 124799069 | 124799575 | 165478 |
| 57233 | 124803960 | 124805501 | 165478 |
| 57234 | 124823118 | 124824461 | 149548 |
| 57235 | 124932136 | 124933572 | 134955 |
| 57236 | 124980802 | 124982314 | 150433 |
| 57237 | 124993052 | 124994510 | 187686 |
| 57238 | 125438627 | 125440648 | 149547, 254671 |
| 57239 | 125461131 | 125463889 | 134910, 254671 |
| 57240 | 125495023 | 125496085 | 149554, 134910 |
| 57241 | 125757161 | 125758349 | 198331 |
| 57242 | 125771312 | 125773745 | 109832, 255027, 110060 |
| 57243 | 125837698 | 125838818 | 064309 |
| 57244 | 125853547 | 125854519 | 064309 |
| 57245 | 125896526 | 125897208 | 064309 |
| 57246 | 125930692 | 125933915 | 064309 |
| 57247 | 125949621 | 125951200 | |
| 57248 | 125961792 | 125962355 | |
| 57249 | 125983273 | 125984475 | |
| 57250 | 125985562 | 125986252 | |
| 57251 | 126017497 | 126018765 | |
| 57252 | 126060604 | 126061146 | |
| 57253 | 126066501 | 126068546 | |
| 57254 | 126072265 | 126073317 | 165526 |
| 57255 | 126080703 | 126082655 | 197798, 165526 |
| 57256 | 126137245 | 126139848 | 110074, 182934 |
| 57257 | 126146405 | 126148389 | 243607, 110074 |
| 57258 | 126152023 | 126154288 | 150455 |
| 57259 | 126173262 | 126174508 | 110063, 255062 |
| 57260 | 126195860 | 126199567 | 110063 |
| 57261 | 126204509 | 126205825 | 110063 |
| 57262 | 126225201 | 126227499 | 110080 |
| 57263 | 126496001 | 126496954 | 149571 |
| 57264 | 126524680 | 126526165 | 254607, 149571 |
| 57265 | 126975998 | 126976616 | |
| 57266 | 127510820 | 127511461 | |
| 57267 | 127518592 | 127519651 | |
| 57268 | 127521072 | 127522458 | |
| 57269 | 127891122 | 127892176 | |
| 57270 | 127895681 | 127898080 | |
| 57271 | 127898135 | 127900347 | |
| 57272 | 127952475 | 127953004 | |
| 57273 | 128005262 | 128006036 | |
| 57274 | 128027445 | 128028393 | |
| 57275 | 128031699 | 128032339 | |
| 57276 | 128034084 | 128034723 | |
| 57277 | 128075167 | 128081562 | 272575 |
| 57278 | 128206036 | 128206527 | |
| 57279 | 128229467 | 128229921 | |
| 57280 | 128317556 | 128320166 | |
| 57281 | 128321375 | 128324439 | |
| 57282 | 128325325 | 128328939 | 134954 |
| 57283 | 128336584 | 128338688 | 134954 |
| 57284 | 128342085 | 128342560 | 134954 |
| 57285 | 128358148 | 128359562 | 134954 |
| 57286 | 128360761 | 128363339 | 134954 |
| 57287 | 128365639 | 128368402 | 134954 |
| 57288 | 128376088 | 128379580 | 134954 |
| 57289 | 128390728 | 128394486 | 134954 |
| 57290 | 128401849 | 128402653 | 134954 |
| 57291 | 128405126 | 128406228 | 134954 |
| 57292 | 128418075 | 128419260 | 134954 |
| 57293 | 128449658 | 128450211 | 134954 |
| 57294 | 128493902 | 128495005 | |
| 57295 | 128577900 | 128579874 | 151702 |
| 57296 | 128764227 | 128765294 | 120457 |
| 57297 | 128985467 | 128986041 | 134909 |
| 57298 | 129013030 | 129014216 | 134909 |
| 57299 | 129145282 | 129146217 | 134909 |
| 57300 | 129148291 | 129150431 | 134909 |
| 57301 | 129154667 | 129155027 | |
| 57302 | 129172241 | 129173624 | |
| 57303 | 129178000 | 129178915 | |
| 57304 | 129193449 | 129196070 | |
| 57305 | 129233600 | 129234595 | |
| 57306 | 129309819 | 129310601 | 043039 |
| 57307 | 129572912 | 129573562 | |
| 57308 | 129601133 | 129601696 | |
| 57309 | 129723227 | 129724155 | 151715 |
| 57310 | 129731597 | 129732693 | |
| 57311 | 129764549 | 129765716 | 170322 |
| 57312 | 129829665 | 129831123 | 170325 |
| 57313 | 129835986 | 129836707 | 170325 |
| 57314 | 129868753 | 129874332 | 233220, 170325 |
| 57315 | 129938212 | 129941187 | 084234, 267981 |
| 57316 | 129993269 | 129995105 | 084234 |
| 57317 | 130013803 | 130014744 | 084234 |
| 57318 | 130016104 | 130016948 | |
| 57319 | 130033329 | 130034299 | 149418 |
| 57320 | 130048124 | 130050619 | 149418 |
| 57321 | 130050635 | 130052390 | 149418 |
| 57322 | 130183388 | 130185665 | 175773, 196323 |
| 57323 | 130188102 | 130189059 | 175773 |
| 57324 | 130266803 | 130267380 | |
| 57325 | 130304257 | 130305501 | |
| 57326 | 130505663 | 130507409 | |
| 57327 | 130522783 | 130523253 | |
| 57328 | 130523654 | 130524321 | |
| 57329 | 130731636 | 130732560 | 254842 |
| 57330 | 130734800 | 130736668 | 255455, 254842 |
| 57331 | 130751487 | 130753987 | 120451 |
| 57332 | 130760809 | 130762327 | 120451 |
| 57333 | 130766414 | 130768761 | 120451 |
| 57334 | 130785457 | 130786849 | 120451 |
| 57335 | 132574604 | 132575163 | 183715 |
| 57336 | 132585845 | 132586390 | 183715 |
| 57337 | 132764197 | 132765074 | 183715 |
| 57338 | 132767670 | 132768608 | 183715 |
| 57339 | 132795552 | 132796507 | 183715 |
| 57340 | 132922330 | 132923110 | 183715 |
| 57341 | 132933740 | 132934952 | 183715 |
| 57342 | 132952553 | 132953147 | 183715 |
| 57343 | 133390267 | 133391442 | 183715 |
| 57344 | 134093669 | 134095643 | 151502, 151503 |
| 57345 | 134120558 | 134120949 | 151500 |
| 57346 | 134122308 | 134124260 | 151498, 151500 |
| 57347 | 134267300 | 134268449 | 109956 |
| 57348 | 134611734 | 134612545 | 251226 |

The locations of ODSCs on Chromosome 12 are shown in Table 54.

TABLE 54
ODSC Locations on Chromosome 12

| SEQ ID NO. | Start | End | Overlapping Gene(s) |
|---|---|---|---|
| 57349 | 275696 | 276481 | 256540, 120645 |
| 57350 | 285451 | 287055 | 256540, 120645 |
| 57351 | 319281 | 320162 | 111181 |
| 57352 | 321727 | 323997 | 111181 |
| 57353 | 345453 | 347461 | 010379 |
| 57354 | 366764 | 367921 | 010379 |
| 57355 | 397701 | 398366 | 073614 |
| 57356 | 497326 | 499532 | 120647, 073614 |
| 57357 | 596921 | 597425 | 139044 |
| 57358 | 678828 | 681409 | 171840 |
| 57359 | 684631 | 686167 | 171840 |
| 57360 | 692251 | 693283 | 171840 |
| 57361 | 694436 | 695357 | 256020, 171840 |
| 57362 | 718850 | 720182 | 171840 |
| 57363 | 736778 | 737971 | 171840 |
| 57364 | 740792 | 743419 | 171840, 177406 |
| 57365 | 751227 | 756411 | 171840, 177406 |
| 57366 | 781991 | 782602 | |
| 57367 | 860104 | 863937 | 060237 |
| 57368 | 887767 | 889783 | 060237 |
| 57369 | 915047 | 915934 | 060237 |
| 57370 | 956430 | 958126 | 060237 |
| 57371 | 1057293 | 1059466 | 002016 |
| 57372 | 1098378 | 1101473 | 082805, 250132, 002016 |
| 57373 | 1123291 | 1123760 | 082805 |
| 57374 | 1163233 | 1164129 | 082805 |
| 57375 | 1239368 | 1239843 | 082805 |
| 57376 | 1240876 | 1241455 | 082805 |
| 57377 | 1628367 | 1628865 | |
| 57378 | 1702736 | 1704617 | 171823, 111186 |
| 57379 | 1714315 | 1715958 | 111186 |
| 57380 | 1738425 | 1739660 | 111186 |
| 57381 | 1768645 | 1771565 | 266043 |
| 57382 | 1775325 | 1780195 | |
| 57383 | 1799235 | 1801387 | 006831 |
| 57384 | 1869095 | 1871028 | 006831 |
| 57385 | 1893282 | 1894348 | 006831 |
| 57386 | 1904733 | 1906897 | 151062 |
| 57387 | 1913720 | 1914184 | 151062 |
| 57388 | 2101881 | 2104428 | 151065, 151067 |
| 57389 | 2112497 | 2114448 | 203593, 151065, 151067 |
| 57390 | 2272393 | 2273215 | 151067 |
| 57391 | 2277390 | 2279728 | 151067 |
| 57392 | 2375895 | 2376656 | 151067 |
| 57393 | 2903097 | 2905358 | 004478, 256030 |
| 57394 | 2921334 | 2923069 | 111203, 258325 |
| 57395 | 2958721 | 2959531 | 206044, 111203 |
| 57396 | 2985738 | 2987222 | 078246, 111206, 171792 |
| 57397 | 2999251 | 3001272 | 078246 |
| 57398 | 3067461 | 3069556 | 197905 |
| 57399 | 3185424 | 3188192 | 011105 |
| 57400 | 3192338 | 3196306 | 011105 |
| 57401 | 3206081 | 3207877 | 011105 |
| 57402 | 3213596 | 3214532 | 011105 |
| 57403 | 3350687 | 3351520 | 011105 |
| 57404 | 3408989 | 3410363 | 250770 |
| 57405 | 3866331 | 3867048 | 130038 |
| 57406 | 3901769 | 3902417 | 111224 |
| 57407 | 3981404 | 3983373 | 111224, 256862 |
| 57408 | 4067371 | 4069985 | |
| 57409 | 4124081 | 4125496 | |
| 57410 | 4139739 | 4141633 | |
| 57411 | 4385774 | 4386538 | 118971 |
| 57412 | 4429509 | 4431395 | 078237 |
| 57413 | 4647018 | 4648274 | 111247, 047621 |
| 57414 | 4713765 | 4714665 | 272921, 010219 |
| 57415 | 4757430 | 4759341 | 139180, 111254, 272921 |
| 57416 | 5282402 | 5283363 | |
| 57417 | 5541487 | 5543066 | 185652 |
| 57418 | 5605872 | 5608109 | 185652 |
| 57419 | 6167657 | 6168836 | 110799 |
| 57420 | 6179569 | 6180143 | 110799 |
| 57421 | 6274681 | 6275141 | |
| 57422 | 6289255 | 6290462 | |
| 57423 | 6291136 | 6293515 | |
| 57424 | 6308653 | 6310929 | 010278 |
| 57425 | 6325040 | 6325886 | 010278 |
| 57426 | 6376081 | 6376830 | |
| 57427 | 6386915 | 6390733 | |
| 57428 | 6401606 | 6402518 | |
| 57429 | 6419121 | 6420354 | 008323 |
| 57430 | 6443692 | 6447191 | 067182 |
| 57431 | 6448730 | 6451978 | 067182 |
| 57432 | 6454356 | 6455813 | |
| 57433 | 6469095 | 6470282 | 111319 |
| 57434 | 6470852 | 6471387 | 111319 |
| 57435 | 6472140 | 6473713 | 111319 |
| 57436 | 6478997 | 6479603 | 111319 |
| 57437 | 6485252 | 6486296 | 111319, 111321 |
| 57438 | 6492122 | 6494860 | 111321 |
| 57439 | 6560039 | 6562472 | 139192, 215039, 139193 |
| 57440 | 6579013 | 6581000 | 139190 |
| 57441 | 6601636 | 6603213 | 010292, 111639 |
| 57442 | 6640922 | 6645522 | 111640, 255966, 010292 |
| 57443 | 6650957 | 6652480 | 010295 |
| 57444 | 6676558 | 6678125 | 111641 |
| 57445 | 6713781 | 6714215 | 111642 |
| 57446 | 6714863 | 6716810 | 111642 |
| 57447 | 6722646 | 6724356 | |
| 57448 | 6771614 | 6773006 | 219410, 111653 |
| 57449 | 6797296 | 6799679 | 126746 |
| 57450 | 6832386 | 6834227 | 111652, 270068 |
| 57451 | 6861436 | 6863842 | 089693 |
| 57452 | 6874216 | 6877886 | 159335, 089693 |
| 57453 | 6880663 | 6881287 | |
| 57454 | 6884963 | 6885524 | 089692 |
| 57455 | 6887452 | 6889491 | 089692 |
| 57456 | 6960698 | 6962659 | 111667, 111665 |
| 57457 | 6975865 | 6977877 | 111669 |
| 57458 | 6981230 | 6983226 | 010626, 240370, 111671 |
| 57459 | 6989264 | 6990170 | 010626, 111671, 240370 |
| 57460 | 6999378 | 7001254 | 010626 |
| 57461 | 7034255 | 7035843 | 111676 |
| 57462 | 7036221 | 7038622 | 111676 |
| 57463 | 7046110 | 7047666 | 111676 |
| 57464 | 7052401 | 7056173 | 111679, 272173, 238923, |
| | | | 111678 |
| 57465 | 7069912 | 7071960 | 111679 |
| 57466 | 7078707 | 7081084 | 215021, 126749 |
| 57467 | 7089743 | 7090905 | 126749, 111684 |
| 57468 | 7103986 | 7104529 | 126749, 111684, 182326 |
| 57469 | 7106647 | 7107284 | 111684, 182326 |
| 57470 | 7124756 | 7126753 | 111684, 182326 |
| 57471 | 7166988 | 7170689 | 182326 |
| 57472 | 7175406 | 7177019 | 182326 |
| 57473 | 7178773 | 7180314 | |
| 57474 | 7186290 | 7187699 | 159403 |
| 57475 | 7242116 | 7246055 | 139178, 159403 |
| 57476 | 7260255 | 7262209 | 205885, 139178 |
| 57477 | 7274889 | 7277246 | 139194, 205885 |
| 57478 | 7280095 | 7284246 | 256967, 139182, 139194 |
| 57479 | 7304069 | 7304485 | 139182 |
| 57480 | 7341053 | 7343163 | 139197, 255572 |
| 57481 | 7526976 | 7527742 | 177675 |
| 57482 | 7585873 | 7586322 | 177675 |
| 57483 | 7644905 | 7647361 | 177575 |
| 57484 | 7664928 | 7665858 | |
| 57485 | 7741268 | 7741888 | |
| 57486 | 8043176 | 8044420 | 173262 |
| 57487 | 8059599 | 8060159 | |
| 57488 | 8178444 | 8180235 | |
| 57489 | 8184591 | 8186355 | 065970 |
| 57490 | 8233769 | 8235677 | 089818 |
| 57491 | 8850177 | 8851409 | 166532 |
| 57492 | 9101169 | 9103481 | 139187, 003056 |
| 57493 | 9215999 | 9218238 | 245105, 214851 |
| 57494 | 9225576 | 9226114 | 175899 |
| 57495 | 9267513 | 9269115 | 175899 |
| 57496 | 9279518 | 9281534 | 211542, 256937 |
| 57497 | 9284389 | 9286053 | |
| 57498 | 9297393 | 9297791 | |
| 57499 | 9373671 | 9374723 | |
| 57500 | 9396183 | 9398179 | 256069 |
| 57501 | 9741262 | 9741944 | |
| 57502 | 10021856 | 10023283 | 110852, 255882 |
| 57503 | 10078187 | 10078942 | 188393 |
| 57504 | 10305701 | 10307034 | |
| 57505 | 10320275 | 10320719 | 173391 |
| 57506 | 10344445 | 10345058 | |
| 57507 | 10364093 | 10368476 | 255958, 139112 |
| 57508 | 10372836 | 10373363 | 139112 |
| 57509 | 10516576 | 10518548 | 245648 |
| 57510 | 10765227 | 10767153 | 111196 |
| 57511 | 10892710 | 10893166 | |
| 57512 | 10902497 | 10903347 | 256888 |
| 57513 | 11019887 | 11020691 | 111215 |
| 57514 | 11044394 | 11045110 | 111215 |
| 57515 | 11092899 | 11093799 | 212127, 111215 |
| 57516 | 11121335 | 11121852 | 212127, 111215 |
| 57517 | 11323293 | 11325343 | 256537, 212127, 256712, |
| | | | 111215 |
| 57518 | 11334721 | 11335710 | |
| 57519 | 11760224 | 11761292 | |
| 57520 | 11775623 | 11776789 | |
| 57521 | 11801242 | 11804364 | 139083 |
| 57522 | 11857804 | 11858395 | 139083 |
| 57523 | 11921458 | 11921951 | 139083 |
| 57524 | 11974812 | 11976022 | 139083 |
| 57525 | 11986336 | 11987772 | 139083 |
| 57526 | 12037616 | 12039379 | 139083 |
| 57527 | 12185377 | 12186965 | |
| 57528 | 12223970 | 12224605 | 121380 |
| 57529 | 12240829 | 12241921 | 121380 |
| 57530 | 12245414 | 12246458 | 121380 |
| 57531 | 12359550 | 12360352 | 121380, 070018 |
| 57532 | 12365458 | 12366347 | 070018 |
| 57533 | 12368716 | 12370416 | 070018 |
| 57534 | 12374226 | 12374779 | 070018 |
| 57535 | 12418513 | 12420920 | 070018 |
| 57536 | 12461314 | 12462097 | |
| 57537 | 12466703 | 12467523 | |
| 57538 | 12472402 | 12473794 | |
| 57539 | 12502473 | 12504525 | 111261 |
| 57540 | 12509161 | 12511900 | 165714, 205791 |
| 57541 | 12529435 | 12530573 | 165714 |
| 57542 | 12549737 | 12554417 | 165714 |
| 57543 | 12555113 | 12560609 | 165714 |
| 57544 | 12569841 | 12570344 | 165714 |
| 57545 | 12590101 | 12599464 | 165714 |
| 57546 | 12601481 | 12605045 | 271370, 165714 |
| 57547 | 12638787 | 12641597 | 255670, 111266 |
| 57548 | 12641696 | 12643057 | 255670, 111266 |
| 57549 | 12658268 | 12659848 | 111266 |
| 57550 | 12710534 | 12717292 | 111266 |
| 57551 | 12737826 | 12739011 | |
| 57552 | 12745785 | 12746205 | |
| 57553 | 12763878 | 12765752 | 111269 |
| 57554 | 12848923 | 12851108 | 183150 |
| 57555 | 12857287 | 12858042 | |
| 57556 | 12868764 | 12872552 | 111276 |
| 57557 | 12876408 | 12879272 | 178878, 256658 |
| 57558 | 12932400 | 12932992 | 178878 |
| 57559 | 12939083 | 12941076 | 178878 |
| 57560 | 12956811 | 12957789 | 178878 |
| 57561 | 12965898 | 12967056 | 213782, 178878 |
| 57562 | 12972388 | 12973091 | 213782, 178878 |
| 57563 | 12975955 | 12976742 | 213782, 178878 |
| 57564 | 13043229 | 13045206 | 013588 |
| 57565 | 13052423 | 13055432 | 013588 |
| 57566 | 13152194 | 13154914 | 255621, 183935, 013583 |
| 57567 | 13197090 | 13197939 | 084444 |
| 57568 | 13250443 | 13252498 | 084444, 111305 |
| 57569 | 13339269 | 13340932 | |
| 57570 | 13348253 | 13350308 | 134531 |
| 57571 | 13375332 | 13377030 | |
| 57572 | 13394562 | 13395431 | |
| 57573 | 13408505 | 13409357 | |
| 57574 | 13430968 | 13431498 | |
| 57575 | 13543185 | 13543824 | |
| 57576 | 13589331 | 13590385 | |
| 57577 | 13595687 | 13597437 | |
| 57578 | 14094394 | 14095199 | 273079 |
| 57579 | 14103124 | 14104301 | 273079 |
| 57580 | 14106482 | 14107490 | 273079 |
| 57581 | 14373793 | 14374424 | 256084 |
| 57582 | 14392167 | 14393313 | |
| 57583 | 14409295 | 14411753 | |
| 57584 | 14518129 | 14520861 | 171681, 244573 |
| 57585 | 14537081 | 14538979 | 171681 |
| 57586 | 14594921 | 14595768 | 171681 |
| 57587 | 14713793 | 14714521 | 121316 |
| 57588 | 14720107 | 14721383 | 256751, 121316 |
| 57589 | 14803552 | 14804170 | 070019 |
| 57590 | 14921779 | 14924447 | 197837 |
| 57591 | 14926573 | 14928865 | 246705 |
| 57592 | 14955530 | 14957657 | 179256, 182993, 084463 |
| 57593 | 14986256 | 14997344 | 256339, 111339, 182993 |
| 57594 | 15780710 | 15783362 | 151491 |
| 57595 | 15865040 | 15866933 | 151491 |
| 57596 | 15885533 | 15885958 | 151491 |
| 57597 | 15910811 | 15911861 | 151491 |
| 57598 | 15940830 | 15943647 | 151491 |
| 57599 | 15948588 | 15951865 | 151491 |
| 57600 | 15972647 | 15973354 | 256953, 151491 |
| 57601 | 15976340 | 15977008 | 256953, 151491 |
| 57602 | 15983006 | 15984000 | 151491 |
| 57603 | 16015609 | 16016620 | 151491 |
| 57604 | 16034361 | 16036518 | 023734, 151491 |
| 57605 | 16063427 | 16065489 | 023697 |
| 57606 | 16080699 | 16082583 | 023697 |
| 57607 | 16084228 | 16085386 | 023697 |
| 57608 | 16108779 | 16109810 | 023697 |
| 57609 | 16114897 | 16117475 | 023697 |
| 57610 | 16131072 | 16133448 | 023697 |
| 57611 | 16183593 | 16184170 | 023697 |
| 57612 | 16391571 | 16392758 | 188991 |
| 57613 | 16433355 | 16434921 | |
| 57614 | 16496787 | 16498923 | |
| 57615 | 16499148 | 16504564 | 008394 |
| 57616 | 16645621 | 16646976 | 008394 |
| 57617 | 17092544 | 17093401 | |
| 57618 | 18081648 | 18082529 | |
| 57619 | 18261334 | 18262113 | 111404 |
| 57620 | 18298863 | 18299769 | 111404 |
| 57621 | 18301665 | 18302588 | 111404 |
| 57622 | 18318140 | 18319142 | 111404 |
| 57623 | 18392028 | 18393018 | 111404 |
| 57624 | 18395066 | 18398678 | 111404 |
| 57625 | 18412531 | 18413687 | 139144, 111404 |
| 57626 | 18472647 | 18473464 | 111404, 139144 |
| 57627 | 18535749 | 18537221 | 139144 |
| 57628 | 18555855 | 18556789 | 139144 |
| 57629 | 18581236 | 18582041 | 139144 |
| 57630 | 18632097 | 18632603 | 139144 |
| 57631 | 18699339 | 18699802 | 139144 |
| 57632 | 18900329 | 18901570 | |
| 57633 | 18906668 | 18907542 | |
| 57634 | 19218908 | 19220057 | |
| 57635 | 19281452 | 19285163 | 052126 |
| 57636 | 19339423 | 19341073 | 052126 |
| 57637 | 19373725 | 19374852 | 052126 |
| 57638 | 19426151 | 19427455 | 052126 |
| 57639 | 19534189 | 19535944 | |
| 57640 | 19584024 | 19585719 | 139154 |
| 57641 | 19592005 | 19593956 | 139154 |
| 57642 | 19682232 | 19684669 | 139154 |
| 57643 | 19799349 | 19800031 | 139154 |
| 57644 | 20098802 | 20099773 | 255910 |
| 57645 | 20122747 | 20123606 | 255910 |
| 57646 | 20141204 | 20141896 | 255910 |
| 57647 | 20150545 | 20153779 | 255910 |
| 57648 | 20164044 | 20164547 | |
| 57649 | 20341868 | 20342706 | |
| 57650 | 20618709 | 20619299 | 172572 |
| 57651 | 20704205 | 20704675 | 256663, 172572 |
| 57652 | 20782308 | 20783199 | 172572 |
| 57653 | 20983344 | 20983943 | 257046, 111700, 205754 |
| 57654 | 21036938 | 21037997 | 257046, 111700, 205754 |
| 57655 | 21282025 | 21284836 | 134538, 257062 |
| 57656 | 21308507 | 21309447 | 134538, 257062 |
| 57657 | 21503818 | 21504562 | 084453 |
| 57658 | 21589826 | 21591678 | 121350 |
| 57659 | 21653598 | 21655549 | 111711, 004700 |
| 57660 | 21679709 | 21681200 | 134548 |
| 57661 | 21699558 | 21700228 | 111713 |
| 57662 | 21712742 | 21714682 | 111713 |
| 57663 | 21718650 | 21720360 | 111713 |
| 57664 | 21751118 | 21752999 | 111713 |
| 57665 | 21757077 | 21758296 | 111713 |
| 57666 | 21909618 | 21911199 | 111716, 256615 |
| 57667 | 21925780 | 21928936 | 121361 |
| 57668 | 21941076 | 21941900 | |
| 57669 | 21952936 | 21953492 | 069431 |
| 57670 | 21967187 | 21968018 | 069431 |
| 57671 | 21971265 | 21973794 | 069431 |
| 57672 | 21975578 | 21977071 | 069431 |
| 57673 | 21981102 | 21982262 | 069431, 257022 |
| 57674 | 22032198 | 22033767 | 069431, 257022 |
| 57675 | 22046171 | 22048470 | 069431 |
| 57676 | 22093384 | 22095765 | 069431 |
| 57677 | 22198889 | 22200965 | 111726 |
| 57678 | 22696131 | 22698070 | 111731 |
| 57679 | 22751638 | 22752127 | |
| 57680 | 22777276 | 22780010 | 139163, 257023 |
| 57681 | 22882167 | 22883682 | 256995 |
| 57682 | 22896960 | 22897671 | 256995 |
| 57683 | 23127829 | 23128262 | 256995 |
| 57684 | 23646501 | 23646988 | |
| 57685 | 23678412 | 23678976 | |
| 57686 | 23923994 | 23925455 | 134532 |
| 57687 | 24102192 | 24104747 | 134532 |
| 57688 | 24190586 | 24192773 | |
| 57689 | 24197533 | 24199601 | |
| 57690 | 24219600 | 24220280 | |
| 57691 | 24222332 | 24223186 | |
| 57692 | 24224685 | 24225246 | |
| 57693 | 24231114 | 24232327 | |
| 57694 | 24233235 | 24234374 | |
| 57695 | 24354147 | 24354986 | |
| 57696 | 24421524 | 24422222 | 255864 |
| 57697 | 24512878 | 24514436 | 255864 |
| 57698 | 24580531 | 24581324 | 255864 |
| 57699 | 24649273 | 24650338 | 255864 |
| 57700 | 24651852 | 24654054 | 255864 |
| 57701 | 24687805 | 24689182 | 255864 |
| 57702 | 24690906 | 24691840 | 255864 |
| 57703 | 24703833 | 24704910 | 255864 |
| 57704 | 24713580 | 24717336 | 255864 |
| 57705 | 24814441 | 24815198 | |
| 57706 | 25347912 | 25349243 | 205707, 118307 |
| 57707 | 25361388 | 25362288 | 205707, 133703 |
| 57708 | 25395689 | 25396940 | 133703 |
| 57709 | 25402822 | 25404732 | 133703 |
| 57710 | 25410496 | 25411218 | |
| 57711 | 25425701 | 25426663 | |
| 57712 | 25428756 | 25430192 | |
| 57713 | 25439475 | 25440349 | |
| 57714 | 25480833 | 25481799 | |
| 57715 | 25486595 | 25487612 | |
| 57716 | 25537386 | 25540857 | |
| 57717 | 25648412 | 25649276 | 152936 |
| 57718 | 25674445 | 25675549 | 152936 |
| 57719 | 25768007 | 25768379 | 152936 |
| 57720 | 26110183 | 26113209 | 123094, 246695 |
| 57721 | 26145190 | 26145833 | 123094 |
| 57722 | 26150455 | 26151099 | 123094 |
| 57723 | 26208044 | 26209719 | 123094 |
| 57724 | 26249922 | 26250870 | |
| 57725 | 26266241 | 26268280 | |
| 57726 | 26277358 | 26280143 | 256894, 123095, 123096 |
| 57727 | 26305309 | 26307936 | 123096 |
| 57728 | 26427359 | 26428569 | 255750, 123096, 256234 |
| 57729 | 26451609 | 26452677 | 123096, 255750, 256234 |
| 57730 | 26470411 | 26472904 | 255968, 255750, 255858, |
| | | | 256234 |
| 57731 | 26514839 | 26515367 | 123104, 255968 |
| 57732 | 26522448 | 26523575 | 123104, 255968 |
| 57733 | 26608749 | 26609591 | 123104 |
| 57734 | 26646578 | 26647131 | 123104 |
| 57735 | 26676532 | 26677119 | 123104 |
| 57736 | 26782061 | 26785101 | 123104, 234428 |
| 57737 | 26789453 | 26790795 | 123104, 234428 |
| 57738 | 26800365 | 26802514 | 234428, 123104 |
| 57739 | 26821747 | 26822275 | 123104 |
| 57740 | 26876697 | 26877416 | 123104 |
| 57741 | 26889106 | 26890573 | 123104 |
| 57742 | 26897855 | 26898390 | 123104 |
| 57743 | 26903821 | 26904541 | 123104 |
| 57744 | 26921610 | 26922665 | 123104 |
| 57745 | 26939888 | 26940744 | 123104 |
| 57746 | 26983700 | 26984553 | 123104 |
| 57747 | 26984681 | 26987181 | 123104 |
| 57748 | 27006377 | 27007349 | |
| 57749 | 27090263 | 27092559 | 111790, 064102 |
| 57750 | 27164968 | 27168268 | 064115 |
| 57751 | 27174924 | 27176370 | 152944 |
| 57752 | 27315772 | 27317024 | |
| 57753 | 27329362 | 27335434 | |
| 57754 | 27341948 | 27348223 | |
| 57755 | 27360381 | 27361580 | |
| 57756 | 27367213 | 27368049 | |
| 57757 | 27396467 | 27399902 | 211455 |
| 57758 | 27430597 | 27431251 | 211455 |
| 57759 | 27484020 | 27489224 | 029153 |
| 57760 | 27490177 | 27491591 | 029153 |
| 57761 | 27495704 | 27497848 | 029153 |
| 57762 | 27675970 | 27678479 | 110841 |
| 57763 | 27700085 | 27704040 | 248100, 110841 |
| 57764 | 27849888 | 27850841 | 174236, 256377 |
| 57765 | 27862606 | 27864729 | 061794, 256377 |
| 57766 | 27893476 | 27895199 | 061794 |
| 57767 | 27900412 | 27902243 | 061794 |
| 57768 | 27907692 | 27908160 | 061794 |
| 57769 | 27932334 | 27934270 | 087448, 256747 |
| 57770 | 28019574 | 28020093 | |
| 57771 | 28342591 | 28345049 | 247934, 123106 |
| 57772 | 28355274 | 28356201 | 123106 |
| 57773 | 28492900 | 28493854 | 123106 |
| 57774 | 28722071 | 28723840 | 123106 |
| 57775 | 28727396 | 28729160 | 123106 |
| 57776 | 28931884 | 28933733 | |
| 57777 | 29394733 | 29395562 | 064763 |
| 57778 | 29472962 | 29474186 | 064763 |
| 57779 | 29533006 | 29535130 | 087502 |
| 57780 | 29581079 | 29582308 | 257599, 187950 |
| 57781 | 29591709 | 29592751 | 257599, 187950 |
| 57782 | 29735572 | 29736266 | 133687 |
| 57783 | 29792667 | 29794415 | 133687 |
| 57784 | 29935222 | 29937582 | 133687 |
| 57785 | 29949537 | 29950147 | |
| 57786 | 30178154 | 30178616 | |
| 57787 | 30178917 | 30180353 | |
| 57788 | 30570493 | 30572549 | |
| 57789 | 30847915 | 30849512 | 133704 |
| 57790 | 30907119 | 30909037 | 246331, 110888 |
| 57791 | 30948543 | 30949505 | 235884 |
| 57792 | 30975063 | 30976477 | |
| 57793 | 31381931 | 31383077 | |
| 57794 | 31476325 | 31479575 | 177340, 139146 |
| 57795 | 31629334 | 31629739 | 170456 |
| 57796 | 31644505 | 31645135 | 170456 |
| 57797 | 31741914 | 31745059 | 255867, 170456 |
| 57798 | 31805776 | 31806783 | 139160 |
| 57799 | 31811705 | 31813475 | 139160 |
| 57800 | 31816433 | 31817664 | 139160 |
| 57801 | 31821138 | 31821666 | 139160 |
| 57802 | 31880957 | 31883206 | 151743 |
| 57803 | 31885593 | 31886449 | |
| 57804 | 31899547 | 31900229 | |
| 57805 | 31901730 | 31902953 | |
| 57806 | 31925961 | 31927129 | |
| 57807 | 31967987 | 31968589 | |
| 57808 | 32006203 | 32007520 | 256372 |
| 57809 | 32013287 | 32014817 | |
| 57810 | 32021948 | 32022536 | |
| 57811 | 32101662 | 32102401 | 242314 |
| 57812 | 32111165 | 32113270 | 174718 |
| 57813 | 32259162 | 32260656 | 151746, 257530 |
| 57814 | 32268588 | 32269191 | 151746 |
| 57815 | 32424076 | 32424820 | 151746 |
| 57816 | 32551833 | 32554033 | 139132 |
| 57817 | 32588897 | 32590936 | 139132 |
| 57818 | 32597327 | 32601298 | 139132 |
| 57819 | 32606807 | 32607863 | 139132 |
| 57820 | 32655063 | 32656357 | 139132 |
| 57821 | 32686672 | 32687601 | 139132 |
| 57822 | 32694902 | 32696679 | 139132 |
| 57823 | 32701469 | 32702180 | 139132 |
| 57824 | 32830610 | 32831392 | |
| 57825 | 32831839 | 32833405 | 087470 |
| 57826 | 32907336 | 32909716 | 139131 |
| 57827 | 33018400 | 33019581 | 057294 |
| 57828 | 33030364 | 33030987 | 057294 |
| 57829 | 33044690 | 33045298 | 057294 |
| 57830 | 33046382 | 33046814 | 057294 |
| 57831 | 33048315 | 33051008 | 057294 |
| 57832 | 33054303 | 33055425 | |
| 57833 | 33059244 | 33059844 | |
| 57834 | 34371099 | 34372058 | 255652 |
| 57835 | 38518185 | 38518881 | |
| 57836 | 38532009 | 38532741 | |
| 57837 | 38710035 | 38710927 | 175548 |
| 57838 | 39273796 | 39274660 | 139117 |
| 57839 | 39277533 | 39277976 | 139117 |
| 57840 | 39279576 | 39280421 | 139117 |
| 57841 | 39283205 | 39284396 | 139117 |
| 57842 | 39285552 | 39287258 | 139117 |
| 57843 | 39298537 | 39301149 | 257718, 139117 |
| 57844 | 39630389 | 39631291 | |
| 57845 | 39734807 | 39735665 | 139116 |
| 57846 | 39742037 | 39743441 | 139116 |
| 57847 | 39813796 | 39814494 | 139116 |
| 57848 | 39820537 | 39821602 | 139116 |
| 57849 | 39835192 | 39838603 | 252974, 139116 |
| 57850 | 39884229 | 39885117 | |
| 57851 | 39986004 | 39986781 | 173208 |
| 57852 | 40040671 | 40041240 | 180116 |
| 57853 | 40218148 | 40219113 | 180116, 151229 |
| 57854 | 40224410 | 40226838 | 180116, 151229 |
| 57855 | 40395808 | 40397071 | 151229 |
| 57856 | 40498751 | 40500210 | 151229 |
| 57857 | 40543665 | 40544132 | |
| 57858 | 40617588 | 40619606 | 225342, 188906 |
| 57859 | 42103131 | 42104038 | |
| 57860 | 42528786 | 42529433 | 151233 |
| 57861 | 42537517 | 42539363 | 151233 |
| 57862 | 42611499 | 42611849 | 015153 |
| 57863 | 42628551 | 42629667 | 015153 |
| 57864 | 42630757 | 42632806 | 134283, 015153 |
| 57865 | 42719111 | 42720800 | 139168, 134283 |
| 57866 | 42877691 | 42878345 | 139174 |
| 57867 | 44152125 | 44153616 | 198001, 129317 |
| 57868 | 44199085 | 44201189 | 151239 |
| 57869 | 44229269 | 44230777 | 139173 |
| 57870 | 44690179 | 44690956 | 139173 |
| 57871 | 45342236 | 45342895 | |
| 57872 | 45586011 | 45586499 | 134297 |
| 57873 | 45594023 | 45594810 | 257738, 134297 |
| 57874 | 45597429 | 45598076 | 134297 |
| 57875 | 45598562 | 45599093 | 134297 |
| 57876 | 45608598 | 45611900 | 177119, 134297 |
| 57877 | 45624878 | 45630209 | 177119 |
| 57878 | 45653534 | 45654799 | 177119 |
| 57879 | 45662454 | 45663540 | 177119 |
| 57880 | 45675984 | 45676720 | 177119 |
| 57881 | 45677325 | 45678794 | 177119 |
| 57882 | 45689910 | 45690419 | 177119 |
| 57883 | 45722912 | 45723469 | 177119 |
| 57884 | 45736846 | 45737556 | 177119 |
| 57885 | 45800977 | 45802645 | 177119 |
| 57886 | 45809064 | 45810197 | 177119 |
| 57887 | 45815354 | 45815875 | 177119 |
| 57888 | 46041456 | 46042461 | |
| 57889 | 46054023 | 46054541 | |
| 57890 | 46102009 | 46103394 | |
| 57891 | 46119401 | 46125309 | 273015, 189079 |
| 57892 | 46360159 | 46360834 | 139218 |
| 57893 | 46382875 | 46386528 | 139218 |
| 57894 | 46573434 | 46573819 | |
| 57895 | 46660701 | 46664051 | 111371 |
| 57896 | 46749646 | 46752788 | 134294 |
| 57897 | 46758417 | 46768980 | 258096, 134294 |
| 57898 | 46775857 | 46782168 | 257496, 257261 |
| 57899 | 46782593 | 46783393 | 257261 |
| 57900 | 46804354 | 46804751 | 257261 |
| 57901 | 46823410 | 46825191 | 257261 |
| 57902 | 46825206 | 46826506 | 257261 |
| 57903 | 46835453 | 46840824 | 257261 |
| 57904 | 46849470 | 46850378 | 257261 |
| 57905 | 46860938 | 46863685 | |
| 57906 | 46865805 | 46868444 | |
| 57907 | 46877059 | 46878218 | |
| 57908 | 46884885 | 46889256 | |
| 57909 | 46947040 | 46952279 | 272369 |
| 57910 | 46955969 | 46957146 | 272369 |
| 57911 | 46970748 | 46971877 | 272369 |
| 57912 | 47010326 | 47011430 | 272369 |
| 57913 | 47058030 | 47059218 | |
| 57914 | 47164112 | 47165826 | 139209 |
| 57915 | 47167709 | 47168384 | 139209 |
| 57916 | 47171377 | 47171879 | 139209 |
| 57917 | 47193955 | 47194564 | 139209 |
| 57918 | 47200652 | 47203910 | 139209 |
| 57919 | 47219261 | 47220204 | 139209 |
| 57920 | 47233610 | 47234271 | |
| 57921 | 47315135 | 47316372 | |
| 57922 | 47331514 | 47332780 | |
| 57923 | 47336382 | 47336965 | |
| 57924 | 47351456 | 47353803 | |
| 57925 | 47357512 | 47359532 | |
| 57926 | 47398290 | 47399856 | |
| 57927 | 47400241 | 47401499 | |
| 57928 | 47411560 | 47412130 | |
| 57929 | 47417424 | 47418151 | |
| 57930 | 47606012 | 47606743 | 247774, 179715 |
| 57931 | 47614429 | 47615842 | 179715 |
| 57932 | 47616289 | 47618338 | 179715 |
| 57933 | 47622536 | 47623049 | 179715 |
| 57934 | 47641106 | 47641648 | 257925 |
| 57935 | 47670253 | 47671211 | 257925 |
| 57936 | 47704710 | 47705250 | |
| 57937 | 47706479 | 47707269 | |
| 57938 | 47710268 | 47711470 | |
| 57939 | 47740085 | 47741168 | |
| 57940 | 47770431 | 47773176 | |
| 57941 | 48099067 | 48101176 | 257433, 005175 |
| 57942 | 48108965 | 48109825 | 257433, 111405 |
| 57943 | 48152075 | 48153697 | 079337, 211584 |
| 57944 | 48166611 | 48168213 | 211584 |
| 57945 | 48212801 | 48215496 | 061273 |
| 57946 | 48245878 | 48246377 | 111424 |
| 57947 | 48298291 | 48299478 | 111424 |
| 57948 | 48498710 | 48500885 | 152556, 079387 |
| 57949 | 48743796 | 48745620 | 257735, 167528 |
| 57950 | 49075107 | 49076515 | 139620 |
| 57951 | 49109358 | 49111588 | 129315 |
| 57952 | 49181637 | 49183897 | 257660, 174233 |
| 57953 | 49207870 | 49209722 | 167535 |
| 57954 | 49245131 | 49246966 | 174243 |
| 57955 | 49257242 | 49261784 | 172602 |
| 57956 | 49272330 | 49272829 | |
| 57957 | 49275548 | 49278474 | |
| 57958 | 49318159 | 49319885 | 255863, 139537, 134285, |
| | | | 272822 |
| 57959 | 49349934 | 49351654 | 134287, 272822 |
| 57960 | 49390589 | 49390951 | 181418 |
| 57961 | 49411318 | 49413797 | 167548, 257913, 181929 |
| 57962 | 49452673 | 49455481 | 167548 |
| 57963 | 49503467 | 49505084 | 139636 |
| 57964 | 49523433 | 49525742 | 123416, 258017 |
| 57965 | 49657855 | 49660127 | 258232, 258101, 167553 |
| 57966 | 49660856 | 49663330 | 258232, 167553 |
| 57967 | 49716414 | 49717107 | 135451, 258334 |
| 57968 | 49738504 | 49742770 | 178401 |
| 57969 | 49759878 | 49761861 | 123352 |
| 57970 | 49875863 | 49877026 | 123352 |
| 57971 | 49908882 | 49909513 | 123352 |
| 57972 | 49960908 | 49962843 | 110844, 187778 |
| 57973 | 49976592 | 49977103 | 135436, 110844 |
| 57974 | 50016008 | 50017830 | 110844 |
| 57975 | 50124274 | 50124950 | 139644 |
| 57976 | 50134759 | 50137358 | 139644 |
| 57977 | 50143500 | 50145887 | 139644 |
| 57978 | 50194153 | 50195327 | 167566 |
| 57979 | 50221376 | 50223163 | 258057, 167566 |
| 57980 | 50235909 | 50238143 | 186666 |
| 57981 | 50356673 | 50357354 | 257588, 161798 |
| 57982 | 50410812 | 50411459 | 161800 |
| 57983 | 50418755 | 50419980 | 161800 |
| 57984 | 50450849 | 50451600 | 110881 |
| 57985 | 50468847 | 50470030 | 110881 |
| 57986 | 50477966 | 50479809 | 066117 |
| 57987 | 50496279 | 50496975 | |
| 57988 | 50497578 | 50498142 | 167588 |
| 57989 | 50502958 | 50507079 | 178449, 272368, 167588 |
| 57990 | 50560293 | 50561931 | 139624 |
| 57991 | 50618096 | 50618526 | 050405, 257256 |
| 57992 | 50640125 | 50641550 | 050405 |
| 57993 | 50642599 | 50643747 | 050405 |
| 57994 | 50664900 | 50666297 | 050405 |
| 57995 | 50676701 | 50678191 | 050405 |
| 57996 | 50697664 | 50698804 | 203426, 203427 |
| 57997 | 50705355 | 50706503 | 196082, 203425 |
| 57998 | 50719544 | 50721261 | 185958 |
| 57999 | 50747728 | 50749170 | 185958 |
| 58000 | 50783247 | 50785044 | 185958 |
| 58001 | 50793824 | 50797572 | 161813 |
| 58002 | 50898348 | 50901328 | 066084 |
| 58003 | 50919822 | 50921492 | 066084 |
| 58004 | 50924983 | 50929141 | 066084 |
| 58005 | 50941638 | 50944692 | 066084 |
| 58006 | 50950604 | 50951647 | 066084 |
| 58007 | 50966053 | 50966403 | 066084 |
| 58008 | 51000392 | 51000958 | 066084 |
| 58009 | 51007894 | 51008809 | 066084 |
| 58010 | 51052716 | 51053979 | 066084 |
| 58011 | 51156778 | 51159478 | 123268 |
| 58012 | 51179413 | 51181681 | 123268 |
| 58013 | 51316253 | 51324257 | 185432 |
| 58014 | 51324458 | 51325371 | 185432 |
| 58015 | 51325475 | 51329588 | 185432 |
| 58016 | 51374195 | 51375055 | 110911 |
| 58017 | 51378251 | 51380067 | 110911 |
| 58018 | 51417392 | 51420570 | 110911 |
| 58019 | 51441043 | 51443218 | 050426 |
| 58020 | 51459273 | 51459863 | 110925 |
| 58021 | 51476567 | 51477743 | 110925 |
| 58022 | 51565889 | 51567604 | 135457 |
| 58023 | 51610711 | 51613113 | 184271 |
| 58024 | 51632304 | 51633636 | 183283 |
| 58025 | 51634690 | 51635196 | 183283 |
| 58026 | 51663108 | 51665668 | 183283, 170545 |
| 58027 | 51764577 | 51766852 | 139629 |
| 58028 | 51984011 | 51985431 | 196876 |
| 58029 | 52014883 | 52015422 | 196876 |
| 58030 | 52019464 | 52020430 | 196876 |
| 58031 | 52056167 | 52057997 | 196876 |
| 58032 | 52065844 | 52066560 | 196876 |
| 58033 | 52076770 | 52078294 | 196876 |
| 58034 | 52152951 | 52154051 | 196876 |
| 58035 | 52166636 | 52168584 | 196876 |
| 58036 | 52171211 | 52172297 | 196876 |
| 58037 | 52207342 | 52209305 | |
| 58038 | 52212654 | 52213151 | 261586 |
| 58039 | 52241582 | 52243745 | 259887 |
| 58040 | 52249553 | 52250441 | |
| 58041 | 52257103 | 52258067 | |
| 58042 | 52277281 | 52281076 | |
| 58043 | 52314689 | 52317507 | 139567 |
| 58044 | 52344852 | 52346966 | 135503 |
| 58045 | 52348141 | 52349342 | 135503 |
| 58046 | 52365223 | 52368389 | 135503 |
| 58047 | 52369287 | 52371558 | 135503 |
| 58048 | 52416626 | 52417903 | 123358 |
| 58049 | 52419129 | 52420175 | 123358 |
| 58050 | 52429977 | 52431624 | 123358 |
| 58051 | 52443854 | 52445644 | 123358 |
| 58052 | 52463177 | 52464731 | 123395 |
| 58053 | 52487035 | 52487704 | 257989, 257542 |
| 58054 | 52539515 | 52543363 | |
| 58055 | 52557483 | 52558254 | |
| 58056 | 52561880 | 52562490 | |
| 58057 | 52584175 | 52587634 | 167767 |
| 58058 | 52595892 | 52598027 | |
| 58059 | 52609126 | 52610300 | 258279 |
| 58060 | 52624263 | 52625211 | |
| 58061 | 52637279 | 52638829 | 135480 |
| 58062 | 52640730 | 52644311 | 135477, 170442, 257671, |
| | | | 135480 |
| 58063 | 52671152 | 52676084 | 257830, 170442 |
| 58064 | 52721133 | 52721771 | |
| 58065 | 52729164 | 52730105 | |
| 58066 | 52836146 | 52836885 | |
| 58067 | 52976736 | 52977416 | |
| 58068 | 53273128 | 53274298 | |
| 58069 | 53277831 | 53279349 | 241146 |
| 58070 | 53288824 | 53289816 | |
| 58071 | 53296728 | 53299676 | 170421 |
| 58072 | 53300850 | 53302624 | 170421 |
| 58073 | 53318269 | 53322009 | 170421 |
| 58074 | 53338810 | 53344634 | 111057, 170421 |
| 58075 | 53345007 | 53345895 | 265039, 111057 |
| 58076 | 53386330 | 53387791 | |
| 58077 | 53399011 | 53401810 | 063046 |
| 58078 | 53439790 | 53446722 | 111077, 257337 |
| 58079 | 53446730 | 53448483 | 257337, 111077 |
| 58080 | 53455089 | 53455923 | 111077 |
| 58081 | 53473039 | 53474065 | 167778 |
| 58082 | 53491230 | 53491846 | 167779 |
| 58083 | 53493025 | 53493721 | 167779 |
| 58084 | 53538937 | 53539959 | |
| 58085 | 53551585 | 53552133 | 139631 |
| 58086 | 53563104 | 53564604 | 139631 |
| 58087 | 53565242 | 53565804 | 139631 |
| 58088 | 53568431 | 53569707 | 139631 |
| 58089 | 53573317 | 53575483 | 139651, 139631 |
| 58090 | 53591026 | 53591942 | 139626 |
| 58091 | 53614488 | 53616128 | 172819 |
| 58092 | 53644988 | 53646814 | 182544 |
| 58093 | 53692932 | 53694718 | 139637, 257605, 123349 |
| 58094 | 53714810 | 53715782 | 094914 |
| 58095 | 53717961 | 53719897 | 094914 |
| 58096 | 53738454 | 53739363 | 170374 |
| 58097 | 53764019 | 53766791 | |
| 58098 | 53772196 | 53776053 | 185591 |
| 58099 | 53815133 | 53815850 | |
| 58100 | 53820598 | 53823207 | 135409 |
| 58101 | 53834454 | 53836268 | 257379, 197111, 205352 |
| 58102 | 53845271 | 53847307 | 257379, 197111 |
| 58103 | 53886418 | 53887380 | 139625 |
| 58104 | 53893647 | 53895727 | 139546, 270175, 139625 |
| 58105 | 53931764 | 53932520 | 267281, 170653 |
| 58106 | 53955302 | 53956354 | 267281, 170653 |
| 58107 | 54018997 | 54020667 | 267281, 170653 |
| 58108 | 54052224 | 54053806 | 135390 |
| 58109 | 54068750 | 54071253 | 135390 |
| 58110 | 54119527 | 54122253 | 012822 |
| 58111 | 54201695 | 54202515 | |
| 58112 | 54218909 | 54219622 | |
| 58113 | 54261296 | 54261759 | |
| 58114 | 54582218 | 54583233 | 123415 |
| 58115 | 54652369 | 54654048 | 094916 |
| 58116 | 54672627 | 54675882 | 135486, 094916, 257596, |
| | | | 258344 |
| 58117 | 54717995 | 54719944 | 111481, 258344 |
| 58118 | 54746651 | 54747905 | 258086, 258137 |
| 58119 | 54752048 | 54755031 | 258086, 258137 |
| 58120 | 54772650 | 54773055 | 258086, 258137, 161642 |
| 58121 | 54773354 | 54773919 | 258086, 258137, 161642 |
| 58122 | 54804536 | 54809984 | 258086, 161638, 258137 |
| 58123 | 54810308 | 54814182 | 161638, 258086, 258137 |
| 58124 | 54980960 | 54981557 | 135447 |
| 58125 | 54981840 | 54982779 | 135447 |
| 58126 | 55078539 | 55079510 | 257634 |
| 58127 | 55082166 | 55082883 | |
| 58128 | 55285704 | 55286421 | |
| 58129 | 55326643 | 55330313 | |
| 58130 | 55644687 | 55645174 | |
| 58131 | 56013295 | 56014159 | |
| 58132 | 56039887 | 56040613 | 272837, 258921 |
| 58133 | 56074252 | 56077226 | 170439 |
| 58134 | 56103171 | 56103911 | 135424 |
| 58135 | 56105339 | 56108075 | 135424 |
| 58136 | 56108916 | 56112852 | 135441, 258311, 135424 |
| 58137 | 56112999 | 56115511 | 135437, 135441, 258311 |
| 58138 | 56120647 | 56124199 | 258056, 135404 |
| 58139 | 56210524 | 56212788 | 123353, 205323, 257390 |
| 58140 | 56223089 | 56224722 | 182796, 135392 |
| 58141 | 56240877 | 56242009 | |
| 58142 | 56302311 | 56303479 | 170473 |
| 58143 | 56312641 | 56313289 | 170473 |
| 58144 | 56320084 | 56322426 | 065357, 170473 |
| 58145 | 56333779 | 56334471 | 065357 |
| 58146 | 56359799 | 56361123 | 123374, 258554, 185664 |
| 58147 | 56365891 | 56366306 | 123374, 185664 |
| 58148 | 56366321 | 56369162 | 111540, 123374, 185664 |
| 58149 | 56389808 | 56391759 | 139531 |
| 58150 | 56400616 | 56401877 | 123411 |
| 58151 | 56434816 | 56436947 | 197728, 257449 |
| 58152 | 56472606 | 56476830 | 065361 |
| 58153 | 56497636 | 56499757 | 170515, 257553, 257411 |
| 58154 | 56509273 | 56511430 | 229117 |
| 58155 | 56511595 | 56514155 | 135482, 258345, 139641, |
| | | | 258317, 229117 |
| 58156 | 56514754 | 56515653 | 135482, 139641, 258317 |
| 58157 | 56519765 | 56520421 | 139641, 258317 |
| 58158 | 56521491 | 56523444 | 258317, 139641 |
| 58159 | 56544339 | 56544864 | 257809 |
| 58160 | 56545339 | 56546704 | 196465, 257809 |
| 58161 | 56550981 | 56553832 | 092841, 257809, 196465 |
| 58162 | 56582388 | 56584857 | 258199, 139613 |
| 58163 | 56614675 | 56616775 | 139579, 181852 |
| 58164 | 56617715 | 56618945 | 139579 |
| 58165 | 56623634 | 56624883 | 139540, 139579 |
| 58166 | 56651411 | 56652997 | 139645 |
| 58167 | 56655453 | 56656089 | |
| 58168 | 56660048 | 56661881 | 258260, 135469 |
| 58169 | 56674411 | 56675158 | 062485 |
| 58170 | 56685145 | 56685806 | 144785, 062485 |
| 58171 | 56692955 | 56695394 | 257303, 062485, 144785 |
| 58172 | 56695511 | 56696663 | 144785, 257303 |
| 58173 | 56708431 | 56710981 | 135473, 144785, 257727, |
| | | | 257303 |
| 58174 | 56726391 | 56728968 | 135473 |
| 58175 | 56753461 | 56755955 | 175336, 170581 |
| 58176 | 56756126 | 56756924 | 175336 |
| 58177 | 56857949 | 56858474 | 135517 |
| 58178 | 56861657 | 56863701 | 176422, 135517 |
| 58179 | 56870673 | 56871476 | 135423 |
| 58180 | 56881138 | 56882472 | 135423 |
| 58181 | 56920156 | 56921308 | 076067 |
| 58182 | 57022624 | 57024614 | 076108 |
| 58183 | 57028387 | 57031299 | 076108 |
| 58184 | 57038787 | 57040687 | 207031, 110955 |
| 58185 | 57080243 | 57083326 | 110958 |
| 58186 | 57117585 | 57120162 | 196531 |
| 58187 | 57145556 | 57146444 | 025423, 198056 |
| 58188 | 57152522 | 57154073 | 025423 |
| 58189 | 57156762 | 57158975 | 025423 |
| 58190 | 57162133 | 57162867 | 025423 |
| 58191 | 57195217 | 57196703 | |
| 58192 | 57197319 | 57197780 | |
| 58193 | 57233570 | 57235633 | 258679 |
| 58194 | 57270335 | 57272538 | |
| 58195 | 57277335 | 57279415 | |
| 58196 | 57291512 | 57292331 | |
| 58197 | 57347929 | 57351951 | 139547 |
| 58198 | 57384838 | 57385705 | |
| 58199 | 57395024 | 57396003 | 166860 |
| 58200 | 57399189 | 57400732 | 166860 |
| 58201 | 57402530 | 57403164 | |
| 58202 | 57480304 | 57484050 | 166886, 166881 |
| 58203 | 57495508 | 57496917 | 166888 |
| 58204 | 57503893 | 57506506 | 166888 |
| 58205 | 57520323 | 57525617 | 123384, 166888 |
| 58206 | 57525655 | 57528297 | 166888, 123384 |
| 58207 | 57531398 | 57533387 | 123384 |
| 58208 | 57542282 | 57544184 | 123384 |
| 58209 | 57622469 | 57625420 | 182199 |
| 58210 | 57632405 | 57634548 | 185633 |
| 58211 | 57638758 | 57639682 | 185482 |
| 58212 | 57652542 | 57656296 | 179912, 258830 |
| 58213 | 57672415 | 57675266 | 179912, 258830 |
| 58214 | 57761973 | 57763078 | 179912 |
| 58215 | 57822937 | 57825864 | 258001, 179912 |
| 58216 | 57827487 | 57829809 | 175189, 258001 |
| 58217 | 57832356 | 57834125 | 175189 |
| 58218 | 57843832 | 57847187 | 139269, 175189 |
| 58219 | 57847761 | 57856876 | 111087, 139269 |
| 58220 | 57881215 | 57883040 | 123329, 166986 |
| 58221 | 57912563 | 57917640 | 166987, 208028, 175197 |
| 58222 | 57939653 | 57941340 | 175203 |
| 58223 | 57984247 | 57985662 | 166908 |
| 58224 | 58026130 | 58027144 | 135454 |
| 58225 | 58138009 | 58139691 | 135452 |
| 58226 | 58144936 | 58146567 | 135446 |
| 58227 | 58165296 | 58167273 | 257921, 037897, 123427 |
| 58228 | 58176253 | 58177451 | 123297, 123427, 257921 |
| 58229 | 58187615 | 58188697 | 123297 |
| 58230 | 58238316 | 58241172 | 175215 |
| 58231 | 58258546 | 58259891 | |
| 58232 | 58289546 | 58291175 | 245651 |
| 58233 | 58298870 | 58299589 | |
| 58234 | 58334483 | 58335899 | 166896 |
| 58235 | 58703725 | 58705572 | |
| 58236 | 59157563 | 59160662 | 258231, 257259 |
| 58237 | 59270836 | 59271529 | 139263 |
| 58238 | 59280793 | 59282345 | 139263 |
| 58239 | 59310765 | 59311682 | 139263 |
| 58240 | 59312677 | 59314744 | 257443, 139263 |
| 58241 | 59318465 | 59319527 | 257443 |
| 58242 | 59434863 | 59435797 | 257443 |
| 58243 | 59448573 | 59450180 | 257443 |
| 58244 | 59989289 | 59991814 | 118596 |
| 58245 | 60044343 | 60046699 | 118596 |
| 58246 | 60091916 | 60092993 | 118596 |
| 58247 | 60093352 | 60094163 | 118596 |
| 58248 | 60108460 | 60109858 | 118596 |
| 58249 | 60117943 | 60118573 | 118596 |
| 58250 | 60252262 | 60252816 | |
| 58251 | 60844723 | 60845947 | |
| 58252 | 61239403 | 61240193 | |
| 58253 | 61243339 | 61244157 | |
| 58254 | 61676552 | 61677449 | |
| 58255 | 61695003 | 61696492 | |
| 58256 | 62287882 | 62288543 | 198673 |
| 58257 | 62296144 | 62296635 | 198673 |
| 58258 | 62308875 | 62309832 | 198673 |
| 58259 | 62652687 | 62655155 | 135655, 198673 |
| 58260 | 62685553 | 62686065 | 135655 |
| 58261 | 62859962 | 62861822 | 061987 |
| 58262 | 62926912 | 62928020 | 061987 |
| 58263 | 62995505 | 62998303 | 257354, 199179, 221949 |
| 58264 | 63025393 | 63026469 | |
| 58265 | 63118689 | 63119080 | 111110 |
| 58266 | 63154250 | 63155126 | 111110 |
| 58267 | 63240916 | 63243275 | 111110 |
| 58268 | 63259968 | 63261308 | 111110 |
| 58269 | 63327261 | 63329250 | 111110 |
| 58270 | 63543283 | 63545230 | 166148 |
| 58271 | 63545593 | 63546356 | |
| 58272 | 63600260 | 63601094 | |
| 58273 | 64173321 | 64174733 | 118600, 249753 |
| 58274 | 64199532 | 64200380 | 118600 |
| 58275 | 64236933 | 64240553 | 196935 |
| 58276 | 64255635 | 64257699 | 196935 |
| 58277 | 64283018 | 64284730 | 196935 |
| 58278 | 64294403 | 64296316 | 196935 |
| 58279 | 64310247 | 64313097 | 196935 |
| 58280 | 64349024 | 64350741 | 196935 |
| 58281 | 64481379 | 64484073 | 255886, 196935 |
| 58282 | 64570737 | 64577180 | |
| 58283 | 64614767 | 64616809 | 243024, 174206 |
| 58284 | 64797259 | 64799679 | 184575 |
| 58285 | 64844956 | 64847685 | 183735 |
| 58286 | 64855341 | 64856131 | 183735 |
| 58287 | 65003432 | 65006095 | 153179 |
| 58288 | 65012812 | 65013352 | 153179 |
| 58289 | 65018467 | 65021790 | 153179 |
| 58290 | 65027229 | 65029071 | 153179 |
| 58291 | 65047176 | 65051174 | 256314, 153179 |
| 58292 | 65063122 | 65063950 | 153179 |
| 58293 | 65073210 | 65074808 | 153179 |
| 58294 | 65140444 | 65141445 | 135677 |
| 58295 | 65152368 | 65154316 | 215159, 135677 |
| 58296 | 65174059 | 65174950 | 111490 |
| 58297 | 65189771 | 65191506 | 111490 |
| 58298 | 65204389 | 65205184 | 111490 |
| 58299 | 65562619 | 65565245 | 174106 |
| 58300 | 65575819 | 65576573 | 174106 |
| 58301 | 65728589 | 65729769 | 174099 |
| 58302 | 65803726 | 65805067 | 174099 |
| 58303 | 65889012 | 65889403 | 250748 |
| 58304 | 65903730 | 65904916 | 250748 |
| 58305 | 66442808 | 66443384 | |
| 58306 | 66523706 | 66525346 | 239335, 139233, 228144, |
| | | | 155957 |
| 58307 | 66538765 | 66539360 | 228144, 155957 |
| 58308 | 66562517 | 66564401 | 228144, 155957 |
| 58309 | 66695679 | 66696948 | 127311 |
| 58310 | 67661750 | 67664572 | 111530 |
| 58311 | 67668828 | 67669877 | 111530 |
| 58312 | 67804085 | 67805008 | |
| 58313 | 67828359 | 67829241 | |
| 58314 | 67986474 | 67987133 | |
| 58315 | 68041041 | 68044458 | 127334 |
| 58316 | 68104271 | 68104939 | 255970 |
| 58317 | 68110482 | 68111128 | 255970 |
| 58318 | 68140900 | 68143858 | |
| 58319 | 68221702 | 68222219 | |
| 58320 | 68726071 | 68726742 | 251301, 111554 |
| 58321 | 68758531 | 68759599 | 251301 |
| 58322 | 68799052 | 68799598 | 251301 |
| 58323 | 68799771 | 68800521 | 251301 |
| 58324 | 68803709 | 68804326 | 251301 |
| 58325 | 68833696 | 68834355 | 251301 |
| 58326 | 68924718 | 68925258 | |
| 58327 | 68925886 | 68927304 | |
| 58328 | 69003417 | 69006918 | 127314 |
| 58329 | 69079471 | 69081744 | 111581, 247363, 241825 |
| 58330 | 69139234 | 69140994 | 175782 |
| 58331 | 69178722 | 69183639 | 175782 |
| 58332 | 69198150 | 69204039 | 256678, 135679 |
| 58333 | 69229194 | 69229807 | 135679 |
| 58334 | 69244010 | 69245039 | 135678 |
| 58335 | 69291125 | 69293984 | 135678 |
| 58336 | 69325679 | 69327614 | 135678 |
| 58337 | 69332490 | 69333797 | 135678 |
| 58338 | 69366151 | 69366954 | |
| 58339 | 69369695 | 69371405 | |
| 58340 | 69417383 | 69419616 | |
| 58341 | 69439895 | 69441056 | |
| 58342 | 69446128 | 69450033 | |
| 58343 | 69460197 | 69461582 | |
| 58344 | 69461598 | 69463247 | |
| 58345 | 69495164 | 69495893 | |
| 58346 | 69632869 | 69634715 | 111605 |
| 58347 | 69677920 | 69678500 | 257818 |
| 58348 | 69724367 | 69726322 | |
| 58349 | 69726588 | 69727212 | |
| 58350 | 69799201 | 69799697 | |
| 58351 | 69809472 | 69810678 | |
| 58352 | 69811226 | 69812271 | |
| 58353 | 69863716 | 69865426 | 166225 |
| 58354 | 69867820 | 69868324 | 166225 |
| 58355 | 69868958 | 69869652 | 166225 |
| 58356 | 69886900 | 69887389 | 166225 |
| 58357 | 69978840 | 69980224 | 166226 |
| 58358 | 70049246 | 70050128 | 127325 |
| 58359 | 70087865 | 70088519 | 127325 |
| 58360 | 70132195 | 70133947 | 127328, 247131 |
| 58361 | 70234287 | 70235334 | 166268, 258052 |
| 58362 | 70381307 | 70382142 | 257815 |
| 58363 | 70446669 | 70447319 | 257815 |
| 58364 | 70635734 | 70638508 | 111596, 257815 |
| 58365 | 71105523 | 71106079 | 153233 |
| 58366 | 71439712 | 71440381 | 257454 |
| 58367 | 71551545 | 71554316 | 127324 |
| 58368 | 71556257 | 71558269 | 127324 |
| 58369 | 71791152 | 71791913 | 127324 |
| 58370 | 71832681 | 71833170 | 127324 |
| 58371 | 71834351 | 71835230 | 139292, 127324 |
| 58372 | 71946736 | 71947400 | 139292 |
| 58373 | 72055836 | 72059026 | 173451, 133858 |
| 58374 | 72075868 | 72076805 | 258064 |
| 58375 | 72079151 | 72080507 | 139291, 258064 |
| 58376 | 72120135 | 72121053 | |
| 58377 | 72148169 | 72150296 | 080371 |
| 58378 | 72232765 | 72234864 | 121749 |
| 58379 | 72340990 | 72341927 | 139287 |
| 58380 | 72348253 | 72349408 | 139287 |
| 58381 | 72350813 | 72353360 | 139287 |
| 58382 | 72668767 | 72669269 | 072657 |
| 58383 | 72905188 | 72906099 | 072657 |
| 58384 | 73470460 | 73471674 | |
| 58385 | 73505171 | 73506875 | |
| 58386 | 73618732 | 73621731 | |
| 58387 | 73746489 | 73747039 | |
| 58388 | 73777336 | 73778487 | |
| 58389 | 73924600 | 73925921 | |
| 58390 | 74930939 | 74932734 | 257386, 253719 |
| 58391 | 75283259 | 75284363 | |
| 58392 | 75357091 | 75358516 | |
| 58393 | 75377634 | 75378024 | |
| 58394 | 75723580 | 75724583 | 180881 |
| 58395 | 75727940 | 75728573 | 173401, 180881 |
| 58396 | 75783681 | 75785963 | 180481, 180881 |
| 58397 | 75873062 | 75876519 | 139278 |
| 58398 | 75904501 | 75906045 | 111615 |
| 58399 | 76044351 | 76045534 | 258077 |
| 58400 | 76049545 | 76051454 | 258077 |
| 58401 | 76059618 | 76061257 | 258077 |
| 58402 | 76118435 | 76119659 | 258077 |
| 58403 | 76129544 | 76130237 | 258077 |
| 58404 | 76144391 | 76146239 | 258077 |
| 58405 | 76178180 | 76178837 | 258077 |
| 58406 | 76255576 | 76258424 | 258077 |
| 58407 | 76302621 | 76303465 | 258077 |
| 58408 | 76327639 | 76329116 | 258077 |
| 58409 | 76337204 | 76339794 | 258077 |
| 58410 | 76344947 | 76348384 | 258077 |
| 58411 | 76369723 | 76374141 | 258077 |
| 58412 | 76375991 | 76377742 | 258077 |
| 58413 | 76379809 | 76380644 | |
| 58414 | 76383940 | 76384732 | |
| 58415 | 76422278 | 76427512 | 257839, 257453, 139289 |
| 58416 | 76477083 | 76479563 | 187109 |
| 58417 | 76659820 | 76661304 | 257219 |
| 58418 | 76670171 | 76670994 | 257219 |
| 58419 | 76678925 | 76679591 | 257219 |
| 58420 | 76693072 | 76694465 | 257219 |
| 58421 | 76741087 | 76742999 | 179941 |
| 58422 | 76849315 | 76849973 | 091039 |
| 58423 | 76952485 | 76954681 | 091039 |
| 58424 | 76977081 | 76978228 | 257526 |
| 58425 | 77157125 | 77159013 | 186908 |
| 58426 | 77271845 | 77273606 | 257910, 175183 |
| 58427 | 77459351 | 77460197 | 165891 |
| 58428 | 78235983 | 78236852 | 067798 |
| 58429 | 78287115 | 78288751 | 067798 |
| 58430 | 78333067 | 78334820 | 067798 |
| 58431 | 78683872 | 78685780 | |
| 58432 | 78685996 | 78688542 | |
| 58433 | 79160912 | 79161672 | |
| 58434 | 79813600 | 79815523 | 067715, 257894 |
| 58435 | 79816719 | 79819323 | 067715, 257894 |
| 58436 | 79939035 | 79944600 | 257474 |
| 58437 | 79948832 | 79949931 | |
| 58438 | 79952760 | 79955035 | 257956 |
| 58439 | 79959586 | 79961176 | |
| 58440 | 80028746 | 80029577 | 177425 |
| 58441 | 80078763 | 80087042 | 258048, 177425 |
| 58442 | 80327550 | 80330035 | 257557, 058272 |
| 58443 | 80580347 | 80580950 | |
| 58444 | 80866586 | 80867433 | 139304 |
| 58445 | 81090643 | 81091758 | |
| 58446 | 81095786 | 81098759 | |
| 58447 | 81170544 | 81173374 | |
| 58448 | 81231073 | 81231719 | 111052 |
| 58449 | 81231774 | 81233104 | 111052 |
| 58450 | 81288421 | 81290356 | 111052 |
| 58451 | 81330032 | 81333041 | 111058, 111052 |
| 58452 | 81471031 | 81474255 | 111058 |
| 58453 | 81479658 | 81480565 | 111058 |
| 58454 | 81514332 | 81515312 | 111058, 258026 |
| 58455 | 81534735 | 81535391 | 111058 |
| 58456 | 81536679 | 81537391 | 111058 |
| 58457 | 81540473 | 81541078 | 111058 |
| 58458 | 81602870 | 81604215 | 111058 |
| 58459 | 82751162 | 82753148 | 127720, 133773 |
| 58460 | 82961473 | 82962264 | |
| 58461 | 83002212 | 83003647 | |
| 58462 | 83044230 | 83044770 | |
| 58463 | 83079363 | 83082121 | 179104 |
| 58464 | 83869718 | 83871591 | |
| 58465 | 87105334 | 87107289 | 258185, 182050 |
| 58466 | 87833642 | 87834423 | |
| 58467 | 87993130 | 87993742 | |
| 58468 | 88055451 | 88056202 | |
| 58469 | 88080402 | 88081713 | |
| 58470 | 88122592 | 88123463 | |
| 58471 | 88149152 | 88150441 | |
| 58472 | 88172520 | 88174422 | |
| 58473 | 88195322 | 88197163 | 257912 |
| 58474 | 88205512 | 88210760 | 257940 |
| 58475 | 88212035 | 88212861 | |
| 58476 | 88214085 | 88215966 | 258179 |
| 58477 | 88220711 | 88222451 | |
| 58478 | 88428737 | 88430485 | 133641 |
| 58479 | 88477007 | 88477877 | 198707 |
| 58480 | 88534986 | 88536913 | 139324, 198707 |
| 58481 | 88641049 | 88641477 | |
| 58482 | 88699163 | 88699879 | |
| 58483 | 88813979 | 88814765 | |
| 58484 | 88973407 | 88974624 | 049130 |
| 58485 | 89140975 | 89141723 | |
| 58486 | 89145690 | 89146586 | |
| 58487 | 89169491 | 89170232 | |
| 58488 | 89319620 | 89320451 | |
| 58489 | 89466183 | 89467881 | 257156 |
| 58490 | 89550575 | 89551946 | 257156 |
| 58491 | 89553704 | 89557931 | 257156 |
| 58492 | 89573740 | 89574210 | 257156 |
| 58493 | 89736651 | 89739188 | |
| 58494 | 89743301 | 89750464 | 139318 |
| 58495 | 89750958 | 89751786 | |
| 58496 | 89766333 | 89770004 | |
| 58497 | 89772666 | 89773732 | |
| 58498 | 89776318 | 89779106 | |
| 58499 | 89779525 | 89780807 | |
| 58500 | 89782671 | 89785922 | |
| 58501 | 89844737 | 89845117 | 139323 |
| 58502 | 89917798 | 89920339 | 270344, 259075, 257594, |
| | | | 139323 |
| 58503 | 90100808 | 90104318 | 271614, 070961 |
| 58504 | 91195522 | 91196107 | |
| 58505 | 91245461 | 91246461 | |
| 58506 | 92171420 | 92173183 | |
| 58507 | 92210864 | 92213283 | |
| 58508 | 92320487 | 92321147 | |
| 58509 | 92428353 | 92429422 | 257242 |
| 58510 | 92456946 | 92460243 | 257242 |
| 58511 | 92463334 | 92465655 | 257242 |
| 58512 | 92529668 | 92532163 | 257242 |
| 58513 | 92536882 | 92540705 | 245904, 133639 |
| 58514 | 92582265 | 92583039 | 245904 |
| 58515 | 92619902 | 92620595 | |
| 58516 | 92833074 | 92833839 | 266923 |
| 58517 | 92895689 | 92901021 | |
| 58518 | 92909813 | 92910626 | |
| 58519 | 92923140 | 92925668 | 258262 |
| 58520 | 92932414 | 92934401 | |
| 58521 | 92939595 | 92942522 | |
| 58522 | 92943865 | 92948669 | |
| 58523 | 92954024 | 92956603 | |
| 58524 | 92961856 | 92969741 | |
| 58525 | 92986150 | 92993014 | |
| 58526 | 92996165 | 92999040 | |
| 58527 | 93017880 | 93019096 | |
| 58528 | 93096479 | 93097586 | 214215 |
| 58529 | 93292380 | 93294844 | 102189 |
| 58530 | 93320957 | 93324516 | 102189 |
| 58531 | 93339483 | 93340174 | |
| 58532 | 93364135 | 93367074 | |
| 58533 | 93367188 | 93368440 | |
| 58534 | 93409552 | 93410350 | 257345, 257322 |
| 58535 | 93481060 | 93481804 | 257322 |
| 58536 | 93515932 | 93516956 | 257322 |
| 58537 | 93520876 | 93522137 | 257322 |
| 58538 | 93528230 | 93529679 | 257322 |
| 58539 | 93591148 | 93592366 | 257322 |
| 58540 | 93637819 | 93638449 | |
| 58541 | 93707802 | 93708931 | 257512 |
| 58542 | 93770759 | 93774136 | 173598, 257252 |
| 58543 | 93834525 | 93836731 | 177889 |
| 58544 | 93860754 | 93862249 | 198015 |
| 58545 | 93878106 | 93878944 | 198015 |
| 58546 | 93920178 | 93921013 | |
| 58547 | 93935443 | 93936035 | |
| 58548 | 93957237 | 93958749 | 246985 |
| 58549 | 93963180 | 93968079 | 120833, 246985 |
| 58550 | 94017803 | 94020173 | |
| 58551 | 94056873 | 94057770 | |
| 58552 | 94070294 | 94074123 | 169372 |
| 58553 | 94075497 | 94077936 | 169372 |
| 58554 | 94094676 | 94096955 | 169372 |
| 58555 | 94099846 | 94102803 | 258274, 169372 |
| 58556 | 94120866 | 94122126 | 169372, 258274 |
| 58557 | 94133296 | 94137076 | 169372 |
| 58558 | 94152768 | 94154288 | 169372 |
| 58559 | 94159856 | 94160653 | 169372 |
| 58560 | 94177383 | 94180830 | 169372 |
| 58561 | 94207398 | 94208704 | 169372 |
| 58562 | 94494949 | 94496633 | |
| 58563 | 94545062 | 94545955 | 136040 |
| 58564 | 94546000 | 94546923 | 136040 |
| 58565 | 94576701 | 94578059 | 136040, 258035 |
| 58566 | 94593855 | 94595436 | 136040 |
| 58567 | 94670279 | 94671569 | 258365, 258172, 136040 |
| 58568 | 94852917 | 94854720 | 173588 |
| 58569 | 94894295 | 94895149 | |
| 58570 | 94928161 | 94929692 | |
| 58571 | 94965171 | 94965805 | 057704 |
| 58572 | 94980366 | 94981011 | 057704 |
| 58573 | 95008741 | 95010534 | 057704 |
| 58574 | 95014715 | 95016767 | 057704 |
| 58575 | 95037312 | 95038751 | 057704 |
| 58576 | 95042381 | 95044600 | 057704 |
| 58577 | 95083933 | 95084599 | |
| 58578 | 95230502 | 95231390 | |
| 58579 | 95396563 | 95397995 | 184752 |
| 58580 | 95466070 | 95467706 | 120798 |
| 58581 | 95479253 | 95479930 | 180263 |
| 58582 | 95490337 | 95492623 | 180263 |
| 58583 | 95508105 | 95513649 | 180263 |
| 58584 | 95563029 | 95563534 | 180263 |
| 58585 | 95592546 | 95593578 | 180263 |
| 58586 | 95610231 | 95613085 | 028203, 180263 |
| 58587 | 95633028 | 95633484 | 028203 |
| 58588 | 95711722 | 95712808 | |
| 58589 | 95790071 | 95791125 | 257583 |
| 58590 | 95839559 | 95841046 | |
| 58591 | 95866693 | 95868917 | 111142 |
| 58592 | 95998794 | 95999289 | |
| 58593 | 96001703 | 96003081 | |
| 58594 | 96005229 | 96006201 | |
| 58595 | 96017151 | 96017802 | |
| 58596 | 96026116 | 96029500 | |
| 58597 | 96046974 | 96048418 | 257150 |
| 58598 | 96155693 | 96156315 | 074527 |
| 58599 | 96182317 | 96185621 | 074527 |
| 58600 | 96189884 | 96191000 | 258343 |
| 58601 | 96195346 | 96201421 | 258292, 258343 |
| 58602 | 96222794 | 96223591 | 258343 |
| 58603 | 96231799 | 96232335 | 258343 |
| 58604 | 96252429 | 96253842 | 139343, 258343 |
| 58605 | 96334252 | 96335015 | 165972 |
| 58606 | 96335735 | 96338196 | 139344, 165972 |
| 58607 | 96359883 | 96361054 | 139344 |
| 58608 | 96369954 | 96370910 | 084110 |
| 58609 | 96388880 | 96390569 | 257878, 084110 |
| 58610 | 96391510 | 96392894 | 257878 |
| 58611 | 96428596 | 96430211 | 111144 |
| 58612 | 96587436 | 96590055 | 111145 |
| 58613 | 96591200 | 96593764 | 111145 |
| 58614 | 96619803 | 96620415 | 111145 |
| 58615 | 96622856 | 96623420 | 111145 |
| 58616 | 96656898 | 96657699 | 111145 |
| 58617 | 96792393 | 96795948 | 059758 |
| 58618 | 96883099 | 96883779 | 188596 |
| 58619 | 96894763 | 96895827 | 188596 |
| 58620 | 97113339 | 97114557 | 188596 |
| 58621 | 97300571 | 97301627 | 139350 |
| 58622 | 97961050 | 97964066 | |
| 58623 | 97965651 | 97966738 | |
| 58624 | 97969089 | 97970101 | |
| 58625 | 98107797 | 98108342 | 257501 |
| 58626 | 98293026 | 98294410 | |
| 58627 | 98306647 | 98307135 | |
| 58628 | 98884313 | 98885542 | 245017 |
| 58629 | 98886141 | 98886821 | 245017 |
| 58630 | 98893126 | 98895577 | 245017 |
| 58631 | 98896694 | 98898362 | 245017 |
| 58632 | 98908555 | 98910599 | 120802, 257167 |
| 58633 | 98967532 | 98969340 | |
| 58634 | 98972206 | 98973158 | |
| 58635 | 98986452 | 98989329 | 075415 |
| 58636 | 99037844 | 99040333 | 120868, 166130 |
| 58637 | 100353173 | 100353887 | 185046 |
| 58638 | 100534955 | 100536965 | 257696, 111647 |
| 58639 | 100548541 | 100549151 | |
| 58640 | 100593038 | 100595284 | 257489, 075089 |
| 58641 | 100607059 | 100607715 | 075089, 166153 |
| 58642 | 100645641 | 100646217 | 166153 |
| 58643 | 100660183 | 100662088 | 136021, 166153 |
| 58644 | 100673318 | 100674064 | 136021 |
| 58645 | 100820378 | 100821696 | |
| 58646 | 100829766 | 100831759 | |
| 58647 | 100838628 | 100839802 | |
| 58648 | 100867296 | 100867929 | 012504 |
| 58649 | 100868322 | 100871032 | 012504 |
| 58650 | 100895646 | 100898197 | 012504 |
| 58651 | 100898480 | 100899078 | 012504 |
| 58652 | 100922299 | 100923756 | 012504 |
| 58653 | 100949308 | 100950197 | 012504 |
| 58654 | 101062626 | 101063178 | |
| 58655 | 101673626 | 101674854 | 120800 |
| 58656 | 101800271 | 101802745 | 257543, 120805 |
| 58657 | 101993640 | 101994387 | 196091 |
| 58658 | 101997834 | 101998680 | 196091 |
| 58659 | 102011920 | 102012824 | 196091 |
| 58660 | 102090245 | 102093109 | 111666 |
| 58661 | 102106885 | 102107902 | 111666 |
| 58662 | 102163635 | 102164070 | 111670 |
| 58663 | 102223472 | 102225130 | 111670 |
| 58664 | 102270296 | 102272676 | 136048 |
| 58665 | 102454907 | 102456038 | 120860 |
| 58666 | 102513088 | 102514519 | 185480, 075188 |
| 58667 | 102566181 | 102566960 | 185480 |
| 58668 | 102736301 | 102736748 | |
| 58669 | 102737096 | 102737897 | |
| 58670 | 102801072 | 102803066 | 017427 |
| 58671 | 102803847 | 102805029 | 017427 |
| 58672 | 102810535 | 102811752 | 017427 |
| 58673 | 102848067 | 102849565 | 017427 |
| 58674 | 102856406 | 102857925 | 017427 |
| 58675 | 102868123 | 102868827 | 017427 |
| 58676 | 102872064 | 102875931 | 017427 |
| 58677 | 102877590 | 102878897 | |
| 58678 | 102904954 | 102906078 | |
| 58679 | 102932886 | 102934309 | |
| 58680 | 102960819 | 102961372 | |
| 58681 | 103046309 | 103046923 | |
| 58682 | 103201876 | 103202825 | |
| 58683 | 103239877 | 103242338 | 171759 |
| 58684 | 103243270 | 103244849 | 171759 |
| 58685 | 103291462 | 103295377 | 171759 |
| 58686 | 103295485 | 103299895 | 171759 |
| 58687 | 103304271 | 103306347 | 171759 |
| 58688 | 103306909 | 103312308 | 171759 |
| 58689 | 103313944 | 103315465 | 171759 |
| 58690 | 103343652 | 103345561 | 171759 |
| 58691 | 103349930 | 103350561 | 171759 |
| 58692 | 103350968 | 103352098 | 139352, 171759 |
| 58693 | 103358540 | 103358989 | |
| 58694 | 103470852 | 103471817 | |
| 58695 | 103487069 | 103488143 | 257703 |
| 58696 | 103508428 | 103509057 | 257703 |
| 58697 | 103521987 | 103523782 | 257703 |
| 58698 | 103555295 | 103555958 | 257703, 257860 |
| 58699 | 103565873 | 103567188 | 257703 |
| 58700 | 103569177 | 103569899 | 257703 |
| 58701 | 103663506 | 103664323 | 179088 |
| 58702 | 103666691 | 103668291 | 179088 |
| 58703 | 103697187 | 103697831 | 179088 |
| 58704 | 103953594 | 103956056 | 257762 |
| 58705 | 103968987 | 103969688 | |
| 58706 | 104066163 | 104067110 | 136011 |
| 58707 | 104178406 | 104180002 | 111696 |
| 58708 | 104193424 | 104194791 | 111696 |
| 58709 | 104198665 | 104199537 | 111696 |
| 58710 | 104234195 | 104236112 | 257327, 111696 |
| 58711 | 104248909 | 104249586 | 214198 |
| 58712 | 104322702 | 104325106 | 265072, 166598, 214198 |
| 58713 | 104349888 | 104351782 | 204954 |
| 58714 | 104358539 | 104361224 | 139372, 204954 |
| 58715 | 104362700 | 104363789 | 139372 |
| 58716 | 104457228 | 104459513 | 111727, 120820 |
| 58717 | 104508254 | 104510227 | |
| 58718 | 104514443 | 104515535 | 120837 |
| 58719 | 104525346 | 104526031 | 120837 |
| 58720 | 104530537 | 104532487 | 120837 |
| 58721 | 104571041 | 104572126 | 257193 |
| 58722 | 104574808 | 104577934 | |
| 58723 | 104580766 | 104582447 | |
| 58724 | 104609213 | 104611171 | 198431 |
| 58725 | 104611941 | 104615171 | 198431 |
| 58726 | 104669261 | 104671846 | 198431, 257732 |
| 58727 | 104672491 | 104675419 | 257732, 198431 |
| 58728 | 104678753 | 104685173 | 198431 |
| 58729 | 104685423 | 104687316 | 198431 |
| 58730 | 104693739 | 104694221 | 198431 |
| 58731 | 104748906 | 104750975 | |
| 58732 | 104802296 | 104802764 | |
| 58733 | 105063479 | 105067031 | 171310 |
| 58734 | 105289192 | 105290830 | 136052 |
| 58735 | 105297752 | 105298299 | 136052 |
| 58736 | 105322919 | 105325690 | 136052 |
| 58737 | 105337867 | 105338989 | 136052 |
| 58738 | 105342312 | 105342858 | 136052 |
| 58739 | 105350984 | 105354284 | 136052 |
| 58740 | 105379735 | 105380928 | 151131 |
| 58741 | 105408189 | 105409136 | 151131 |
| 58742 | 105500596 | 105502712 | 136051, 257999 |
| 58743 | 105514335 | 105514763 | 136051 |
| 58744 | 105588671 | 105589409 | 136044 |
| 58745 | 105606047 | 105606958 | 136044 |
| 58746 | 105608027 | 105609602 | 136044 |
| 58747 | 105628974 | 105630755 | 235162, 136044 |
| 58748 | 105647059 | 105648486 | 235162 |
| 58749 | 105671757 | 105672491 | 235162 |
| 58750 | 105686150 | 105687516 | 235162 |
| 58751 | 105710755 | 105713570 | 257642, 235162 |
| 58752 | 105746576 | 105747197 | 235162 |
| 58753 | 105793336 | 105794342 | |
| 58754 | 106199085 | 106200597 | |
| 58755 | 106415878 | 106417494 | |
| 58756 | 106489515 | 106491261 | 074590 |
| 58757 | 106492091 | 106493376 | 074590 |
| 58758 | 106513529 | 106516577 | 074590 |
| 58759 | 106519034 | 106521688 | 074590 |
| 58760 | 106525377 | 106530618 | 074590 |
| 58761 | 106530846 | 106534500 | 074590 |
| 58762 | 106589202 | 106589835 | |
| 58763 | 106620528 | 106621866 | |
| 58764 | 106640279 | 106642775 | 258355, 136026 |
| 58765 | 106667877 | 106669036 | 136026 |
| 58766 | 106675581 | 106676866 | 238609, 136026 |
| 58767 | 106694363 | 106697644 | 166046, 136026 |
| 58768 | 106700812 | 106701484 | 166046 |
| 58769 | 106736329 | 106737025 | 166046 |
| 58770 | 106751082 | 106753114 | 013503 |
| 58771 | 106763065 | 106764200 | 013503 |
| 58772 | 106811857 | 106813383 | 013503 |
| 58773 | 107025715 | 107028223 | 257545, 111783 |
| 58774 | 107111087 | 107112121 | 257545, 111783, 257918 |
| 58775 | 107158326 | 107159013 | 257545 |
| 58776 | 107159697 | 107164215 | 257545 |
| 58777 | 107164414 | 107166392 | 257545 |
| 58778 | 107166489 | 107169347 | 111785, 257545 |
| 58779 | 107233918 | 107234670 | 111785 |
| 58780 | 107348753 | 107351111 | 151135, 260329 |
| 58781 | 107369786 | 107370847 | 151135 |
| 58782 | 107380129 | 107381461 | 120832 |
| 58783 | 107420070 | 107423061 | 008405 |
| 58784 | 107485410 | 107488826 | 257548, 008405 |
| 58785 | 107495464 | 107496036 | 257548 |
| 58786 | 107737272 | 107740035 | 151136 |
| 58787 | 107741623 | 107746649 | 151136 |
| 58788 | 107773378 | 107774561 | 151136 |
| 58789 | 107908660 | 107909826 | 151136 |
| 58790 | 107914210 | 107915304 | 151136 |
| 58791 | 108025362 | 108026242 | 151136 |
| 58792 | 108078210 | 108080551 | 136045 |
| 58793 | 108152540 | 108155514 | 110851, 258136 |
| 58794 | 108908783 | 108910519 | 198855 |
| 58795 | 108953521 | 108957778 | 136003, 075856 |
| 58796 | 108959681 | 108960780 | 136003 |
| 58797 | 109005604 | 109006207 | |
| 58798 | 109037558 | 109038401 | |
| 58799 | 109058019 | 109059896 | 110880 |
| 58800 | 109059935 | 109060487 | 110880 |
| 58801 | 109084336 | 109086720 | 110880 |
| 58802 | 109122535 | 109126856 | 110880 |
| 58803 | 109127451 | 109129260 | |
| 58804 | 109167577 | 109168324 | |
| 58805 | 109226336 | 109228040 | 084112 |
| 58806 | 109231614 | 109233437 | 084112 |
| 58807 | 109249448 | 109252180 | 084112 |
| 58808 | 109273136 | 109274373 | 110887 |
| 58809 | 109275639 | 109276663 | 110887 |
| 58810 | 109280110 | 109281651 | 110887 |
| 58811 | 109490196 | 109492061 | 256262, 135093 |
| 58812 | 109503100 | 109503776 | 135093 |
| 58813 | 109522083 | 109522822 | 135093 |
| 58814 | 109524785 | 109525603 | 135093 |
| 58815 | 109530251 | 109531657 | 189046 |
| 58816 | 109535623 | 109536346 | 076248 |
| 58817 | 109548586 | 109550216 | 256139, 076248 |
| 58818 | 109553188 | 109555006 | 076555, 256139 |
| 58819 | 109568401 | 109570173 | 076555 |
| 58820 | 109571850 | 109572594 | 076555 |
| 58821 | 109581893 | 109584577 | 076555 |
| 58822 | 109585413 | 109587005 | 076555 |
| 58823 | 109589149 | 109589806 | 076555 |
| 58824 | 109591492 | 109592715 | 076555 |
| 58825 | 109629188 | 109630545 | 076555 |
| 58826 | 109634318 | 109639678 | 076555 |
| 58827 | 109685783 | 109686711 | 076555 |
| 58828 | 109715351 | 109716926 | 139445 |
| 58829 | 109740463 | 109741062 | 139445 |
| 58830 | 109900476 | 109901348 | 110906 |
| 58831 | 109914145 | 109916405 | 151148, 110906 |
| 58832 | 109994956 | 109995443 | 139428 |
| 58833 | 110009446 | 110010051 | 139428 |
| 58834 | 110010290 | 110012070 | 110921, 139428 |
| 58835 | 110016146 | 110016687 | 110921 |
| 58836 | 110024047 | 110025239 | 110921 |
| 58837 | 110026131 | 110027483 | 110921 |
| 58838 | 110035308 | 110036269 | |
| 58839 | 110053441 | 110054220 | |
| 58840 | 110144430 | 110145657 | |
| 58841 | 110148228 | 110149024 | |
| 58842 | 110151252 | 110153352 | 139438 |
| 58843 | 110158915 | 110159772 | 139438 |
| 58844 | 110159789 | 110160477 | 139438 |
| 58845 | 110160968 | 110161770 | 139438 |
| 58846 | 110260337 | 110261805 | 111199 |
| 58847 | 110268085 | 110268688 | 111199 |
| 58848 | 110302030 | 110303135 | 139433 |
| 58849 | 110317239 | 110319075 | 249094, 139433 |
| 58850 | 110337347 | 110339123 | 139437 |
| 58851 | 110386149 | 110387634 | 139436, 139437 |
| 58852 | 110388512 | 110390560 | 139436, 139437 |
| 58853 | 110433107 | 110434827 | 139436 |
| 58854 | 110436440 | 110438498 | 076513 |
| 58855 | 110448688 | 110449313 | 076513 |
| 58856 | 110449687 | 110450481 | 076513 |
| 58857 | 110460979 | 110462973 | 076513 |
| 58858 | 110475857 | 110478112 | 076513, 174456 |
| 58859 | 110479388 | 110481328 | 174456 |
| 58860 | 110485444 | 110486960 | 174456 |
| 58861 | 110528524 | 110529532 | |
| 58862 | 110545666 | 110548339 | |
| 58863 | 110561809 | 110563003 | 122970 |
| 58864 | 110683201 | 110686556 | |
| 58865 | 110717986 | 110720799 | 174437 |
| 58866 | 110750448 | 110751335 | 174437 |
| 58867 | 110840426 | 110842509 | 196510, 258210 |
| 58868 | 110887006 | 110888974 | 111229 |
| 58869 | 110905286 | 110907480 | 204856, 111231 |
| 58870 | 110938986 | 110940461 | 151164, 111237 |
| 58871 | 110971325 | 110971868 | 196850 |
| 58872 | 110974502 | 110976215 | 196850 |
| 58873 | 110981176 | 110981909 | 196850 |
| 58874 | 110989756 | 110990510 | 196850 |
| 58875 | 111012502 | 111013736 | 196850 |
| 58876 | 111015326 | 111017309 | 196850 |
| 58877 | 111019174 | 111022218 | 196850 |
| 58878 | 111028593 | 111030140 | |
| 58879 | 111051487 | 111052401 | 204852 |
| 58880 | 111179261 | 111183073 | 186298 |
| 58881 | 111325561 | 111326247 | 173093 |
| 58882 | 111372090 | 111372776 | |
| 58883 | 111500385 | 111502159 | 111249 |
| 58884 | 111504219 | 111506240 | 111249 |
| 58885 | 111511699 | 111513607 | 221259, 111249 |
| 58886 | 111513969 | 111515781 | 111249 |
| 58887 | 111539615 | 111540516 | 111249 |
| 58888 | 111545086 | 111546252 | 111249 |
| 58889 | 111800650 | 111802442 | 198324 |
| 58890 | 111804997 | 111807250 | 257595, 198324 |
| 58891 | 111834749 | 111835851 | 257595 |
| 58892 | 111841914 | 111845326 | 111252 |
| 58893 | 111850938 | 111852121 | 111252 |
| 58894 | 111863220 | 111864093 | 111252 |
| 58895 | 111867941 | 111870855 | 111252 |
| 58896 | 111872042 | 111873033 | 111252 |
| 58897 | 112034924 | 112038834 | 258099, 204842 |
| 58898 | 112053931 | 112054539 | |
| 58899 | 112074044 | 112074703 | |
| 58900 | 112122769 | 112124812 | 111271, 089234 |
| 58901 | 112190903 | 112191524 | 111271 |
| 58902 | 112203955 | 112206432 | 111275, 257767 |
| 58903 | 112210843 | 112213955 | 257767, 111275 |
| 58904 | 112214563 | 112216366 | 257767, 111275 |
| 58905 | 112218355 | 112219495 | 257767, 111275 |
| 58906 | 112225837 | 112226642 | 257767, 111275 |
| 58907 | 112228734 | 112231122 | 257767, 111275 |
| 58908 | 112279476 | 112281047 | 089022, 234608 |
| 58909 | 112439673 | 112440286 | 198270 |
| 58910 | 112443692 | 112444309 | 198270 |
| 58911 | 112449959 | 112452408 | 089248, 198270 |
| 58912 | 112533903 | 112535365 | 111300 |
| 58913 | 112545070 | 112547432 | 111300 |
| 58914 | 112562872 | 112564531 | 135148 |
| 58915 | 112568063 | 112568938 | 135148 |
| 58916 | 112689846 | 112690449 | 173064 |
| 58917 | 112723260 | 112724194 | 173064 |
| 58918 | 112756861 | 112757716 | 173064 |
| 58919 | 112819015 | 112820617 | 173064 |
| 58920 | 112846279 | 112847941 | 089009 |
| 58921 | 112855899 | 112858124 | 179295, 089009 |
| 58922 | 112903716 | 112904472 | 179295 |
| 58923 | 112908244 | 112909385 | 179295 |
| 58924 | 113037585 | 113038188 | 089169 |
| 58925 | 113276436 | 113277987 | 089169 |
| 58926 | 113343870 | 113345907 | 089127, 257452 |
| 58927 | 113362471 | 113363248 | 089127, 257452 |
| 58928 | 113375563 | 113377038 | 111331, 257452 |
| 58929 | 113415405 | 113417507 | 111335, 257452 |
| 58930 | 113494327 | 113495463 | 135144 |
| 58931 | 113500220 | 113500849 | 135144 |
| 58932 | 113501624 | 113502677 | 135144 |
| 58933 | 113508881 | 113510539 | 135144 |
| 58934 | 113535870 | 113536783 | 111344 |
| 58935 | 113622530 | 113625097 | 257286, 139405, 123064 |
| 58936 | 113631594 | 113632395 | |
| 58937 | 113658870 | 113661989 | 166578, 186815 |
| 58938 | 113667009 | 113670738 | 186815 |
| 58939 | 113673883 | 113675165 | 186815 |
| 58940 | 113678575 | 113680926 | 186815 |
| 58941 | 113683552 | 113685278 | 186815 |
| 58942 | 113697109 | 113697854 | 186815 |
| 58943 | 113709168 | 113710491 | 186815 |
| 58944 | 113771945 | 113773517 | 089060 |
| 58945 | 113795591 | 113797493 | 151176, 089060 |
| 58946 | 113840126 | 113842953 | 135094 |
| 58947 | 113850698 | 113852959 | 135094 |
| 58948 | 113853023 | 113857321 | 135094 |
| 58949 | 113858056 | 113861457 | 139410, 135094 |
| 58950 | 114203580 | 114204526 | 249550 |
| 58951 | 114207962 | 114208821 | 249550 |
| 58952 | 114403175 | 114405019 | 122965 |
| 58953 | 114702749 | 114703398 | |
| 58954 | 114917145 | 114918698 | |
| 58955 | 115049128 | 115050041 | |
| 58956 | 115105502 | 115108023 | |
| 58957 | 115119020 | 115123464 | 135111 |
| 58958 | 115129572 | 115131084 | |
| 58959 | 115174262 | 115174922 | 257817 |
| 58960 | 115237118 | 115238170 | |
| 58961 | 115263972 | 115264640 | |
| 58962 | 115267369 | 115268008 | |
| 58963 | 115355771 | 115357828 | |
| 58964 | 115362879 | 115363601 | |
| 58965 | 115370525 | 115372369 | |
| 58966 | 115447579 | 115448886 | |
| 58967 | 115515911 | 115516794 | 257958 |
| 58968 | 115525925 | 115528014 | |
| 58969 | 115531805 | 115532442 | |
| 58970 | 115573556 | 115574215 | |
| 58971 | 115664983 | 115665507 | |
| 58972 | 115699432 | 115700490 | |
| 58973 | 115751452 | 115752736 | |
| 58974 | 115762987 | 115764461 | |
| 58975 | 115775311 | 115776599 | |
| 58976 | 115778195 | 115780286 | |
| 58977 | 115791267 | 115793089 | |
| 58978 | 115812424 | 115813569 | |
| 58979 | 115861126 | 115862111 | |
| 58980 | 115889450 | 115890152 | |
| 58981 | 116381008 | 116382418 | |
| 58982 | 116391113 | 116391805 | |
| 58983 | 116491289 | 116492173 | 123066 |
| 58984 | 116561258 | 116561788 | 123066 |
| 58985 | 116570834 | 116571838 | 123066 |
| 58986 | 116713837 | 116716326 | 123066 |
| 58987 | 116806329 | 116806630 | 258346 |
| 58988 | 116823423 | 116824041 | 258346 |
| 58989 | 116842673 | 116843856 | |
| 58990 | 116844310 | 116844883 | |
| 58991 | 116874489 | 116875137 | |
| 58992 | 116962605 | 116963803 | |
| 58993 | 117074590 | 117075135 | |
| 58994 | 117096221 | 117096981 | |
| 58995 | 117100214 | 117101240 | 257883 |
| 58996 | 117101308 | 117101767 | 257883 |
| 58997 | 117114434 | 117115170 | 257883 |
| 58998 | 117130663 | 117131661 | 257883 |
| 58999 | 117173950 | 117174517 | 111412 |
| 59000 | 117174761 | 117176201 | 135119, 111412 |
| 59001 | 117256220 | 117257659 | 135119 |
| 59002 | 117348486 | 117349574 | 174989 |
| 59003 | 117536251 | 117537290 | 258285, 088992 |
| 59004 | 117567897 | 117569108 | 258285 |
| 59005 | 117580242 | 117582186 | 135108 |
| 59006 | 117596041 | 117597287 | 135108 |
| 59007 | 117626692 | 117628683 | 135108 |
| 59008 | 117845825 | 117846358 | 089250 |
| 59009 | 117904249 | 117905616 | 171435 |
| 59010 | 117922965 | 117925994 | 171435 |
| 59011 | 117932433 | 117933282 | 171435 |
| 59012 | 117935758 | 117936282 | 171435 |
| 59013 | 117956164 | 117957666 | 171435 |
| 59014 | 118453673 | 118455158 | 111445 |
| 59015 | 118489432 | 118492161 | 176871 |
| 59016 | 118497014 | 118500955 | 176871 |
| 59017 | 118539689 | 118542367 | 176834 |
| 59018 | 118558439 | 118559247 | 176834 |
| 59019 | 118561959 | 118562913 | 176834 |
| 59020 | 118573264 | 118575598 | 089220, 176834 |
| 59021 | 118576495 | 118577428 | 089220 |
| 59022 | 118809182 | 118815411 | 111707, 270482, 135090 |
| 59023 | 119616219 | 119618763 | 152137 |
| 59024 | 120026849 | 120028173 | 248636 |
| 59025 | 120031194 | 120033381 | 224982, 248636 |
| 59026 | 120105275 | 120106888 | 111725, 248636 |
| 59027 | 120118853 | 120119495 | 111725 |
| 59028 | 120153185 | 120155289 | 122966 |
| 59029 | 120426227 | 120427330 | |
| 59030 | 120427563 | 120427977 | 135127 |
| 59031 | 120478921 | 120479821 | 135127 |
| 59032 | 120493054 | 120493897 | 135127 |
| 59033 | 120524048 | 120525430 | 135127 |
| 59034 | 120553908 | 120557210 | 111737 |
| 59035 | 120557260 | 120557873 | |
| 59036 | 120619778 | 120621332 | 089154 |
| 59037 | 120631446 | 120633516 | 089154 |
| 59038 | 120637242 | 120640154 | 255857, 089157 |
| 59039 | 120665371 | 120671227 | 089159 |
| 59040 | 120671781 | 120674018 | 089159 |
| 59041 | 120702519 | 120704594 | 089159 |
| 59042 | 120728366 | 120731659 | 200795, 202538 |
| 59043 | 120739949 | 120740556 | 089163 |
| 59044 | 120754317 | 120755862 | |
| 59045 | 120759141 | 120759574 | |
| 59046 | 120763180 | 120764038 | 170890 |
| 59047 | 120883124 | 120885045 | 257218, 170855, 111780 |
| 59048 | 120906312 | 120908349 | 088986, 111786 |
| 59049 | 120933383 | 120935048 | 248008, 088986 |
| 59050 | 120966046 | 120967986 | 110871 |
| 59051 | 120971855 | 120973350 | 110871, 022840 |
| 59052 | 120975919 | 120977520 | 022840 |
| 59053 | 120987739 | 120988625 | 022840 |
| 59054 | 121018742 | 121019649 | 167272 |
| 59055 | 121124184 | 121127179 | 110917 |
| 59056 | 121129862 | 121130683 | 110917 |
| 59057 | 121147801 | 121149307 | 175970 |
| 59058 | 121163458 | 121164394 | 122971 |
| 59059 | 121263094 | 121263578 | 157837 |
| 59060 | 121340597 | 121342763 | 157837 |
| 59061 | 121382317 | 121383023 | |
| 59062 | 121398343 | 121400903 | |
| 59063 | 121415345 | 121419826 | 135100, 241388 |
| 59064 | 121421449 | 121422921 | 135100 |
| 59065 | 121462728 | 121464834 | 135114 |
| 59066 | 121476277 | 121477354 | 135114 |
| 59067 | 121554520 | 121555185 | |
| 59068 | 121561596 | 121563183 | |
| 59069 | 121564016 | 121564499 | |
| 59070 | 121566538 | 121567516 | |
| 59071 | 121581721 | 121582895 | 089041 |
| 59072 | 121628197 | 121629440 | |
| 59073 | 121647237 | 121649131 | 135124 |
| 59074 | 121651547 | 121653700 | 135124 |
| 59075 | 121665012 | 121665472 | 135124 |
| 59076 | 121722283 | 121722907 | 110931 |
| 59077 | 121733244 | 121735810 | 110931 |
| 59078 | 121789149 | 121791138 | 089053 |
| 59079 | 121814587 | 121815122 | 089053 |
| 59080 | 121837019 | 121839039 | 170633, 089053, 258435 |
| 59081 | 121884557 | 121885033 | 089094 |
| 59082 | 121904309 | 121905508 | 089094 |
| 59083 | 121954126 | 121954929 | 089094 |
| 59084 | 121956116 | 121957081 | 089094 |
| 59085 | 121973734 | 121974590 | 089094 |
| 59086 | 121974730 | 121976923 | 089094 |
| 59087 | 121988726 | 121989294 | 089094 |
| 59088 | 122016971 | 122018737 | 256742, 089094 |
| 59089 | 122063873 | 122065894 | 182500 |
| 59090 | 122124467 | 122125421 | |
| 59091 | 122147543 | 122148126 | |
| 59092 | 122149517 | 122151604 | 188735 |
| 59093 | 122191649 | 122192336 | 188735 |
| 59094 | 122221537 | 122223438 | 139725 |
| 59095 | 122223780 | 122225763 | 139725 |
| 59096 | 122227617 | 122244061 | 272849, 212694, 139718, |
| | | | 139725 |
| 59097 | 122249571 | 122251476 | 139718 |
| 59098 | 122293952 | 122298852 | 256811, 158104 |
| 59099 | 122313969 | 122314984 | |
| 59100 | 122325531 | 122327544 | 110801, 256950 |
| 59101 | 122362530 | 122362993 | 158023 |
| 59102 | 122459422 | 122462678 | 110987 |
| 59103 | 122478400 | 122478985 | 110987 |
| 59104 | 122488677 | 122490613 | 110987 |
| 59105 | 122500371 | 122501895 | 256546 |
| 59106 | 122509818 | 122510989 | |
| 59107 | 122515607 | 122520478 | 175727 |
| 59108 | 122582142 | 122583790 | 175727 |
| 59109 | 122598620 | 122599622 | 175727 |
| 59110 | 122609763 | 122612289 | 175727 |
| 59111 | 122617305 | 122618081 | 175727 |
| 59112 | 122661520 | 122662198 | 158113 |
| 59113 | 122709742 | 122711270 | 184047, 256861 |
| 59114 | 122720256 | 122720704 | 139719, 256861 |
| 59115 | 122750261 | 122751763 | 139719, 256861 |
| 59116 | 122850777 | 122853747 | 130779 |
| 59117 | 122879125 | 122880281 | 257097, 130779 |
| 59118 | 122882655 | 122886343 | 257097, 130779 |
| 59119 | 122889517 | 122892434 | 130779 |
| 59120 | 122903620 | 122908535 | 130779 |
| 59121 | 122948030 | 122949687 | |
| 59122 | 122973359 | 122974426 | 033030 |
| 59123 | 122984323 | 122985911 | 033030 |
| 59124 | 123010362 | 123012136 | 184445, 111011 |
| 59125 | 123163017 | 123164839 | 196917 |
| 59126 | 123210334 | 123210998 | 270134, 196917 |
| 59127 | 123236297 | 123238487 | 139726 |
| 59128 | 123310201 | 123311477 | 130783 |
| 59129 | 123318793 | 123323516 | 130787 |
| 59130 | 123324273 | 123328027 | 130787 |
| 59131 | 123344575 | 123345898 | 130787 |
| 59132 | 123347326 | 123348434 | 130787 |
| 59133 | 123349593 | 123353425 | 256152, 139722 |
| 59134 | 123371401 | 123374095 | 139722 |
| 59135 | 123379280 | 123381217 | 139722 |
| 59136 | 123382640 | 123383914 | |
| 59137 | 123401827 | 123402605 | |
| 59138 | 123429112 | 123432678 | 150967 |
| 59139 | 123458364 | 123461607 | 256028, 111325, 150967 |
| 59140 | 123464424 | 123466673 | 182196, 150967, 111325, |
| | | | 256028 |
| 59141 | 123469015 | 123470002 | 090975 |
| 59142 | 123479571 | 123481468 | 090975 |
| 59143 | 123503057 | 123504874 | 090975 |
| 59144 | 123533705 | 123535822 | 090975 |
| 59145 | 123540431 | 123541008 | 090975 |
| 59146 | 123546382 | 123549836 | 090975 |
| 59147 | 123553797 | 123555636 | 090975 |
| 59148 | 123569398 | 123571150 | 090975 |
| 59149 | 123572965 | 123576363 | 090975 |
| 59150 | 123576737 | 123577279 | 090975 |
| 59151 | 123578049 | 123579887 | 090975 |
| 59152 | 123610395 | 123612377 | 090975 |
| 59153 | 123623826 | 123628215 | 090975 |
| 59154 | 123633852 | 123636708 | 090975 |
| 59155 | 123648814 | 123650686 | 051825 |
| 59156 | 123716547 | 123719193 | 130921, 051825 |
| 59157 | 123754944 | 123757146 | 111328 |
| 59158 | 123777897 | 123778516 | 139697 |
| 59159 | 123847742 | 123850638 | 256092, 139697 |
| 59160 | 123867953 | 123868751 | 183955 |
| 59161 | 123872962 | 123875156 | 183955 |
| 59162 | 123920440 | 123921612 | 150977 |
| 59163 | 123942129 | 123943791 | 184209 |
| 59164 | 124017385 | 124018224 | 188026 |
| 59165 | 124018293 | 124019006 | |
| 59166 | 124055824 | 124056332 | |
| 59167 | 124068537 | 124070085 | 086598, 247373 |
| 59168 | 124080287 | 124080805 | 086598 |
| 59169 | 124085839 | 124087623 | 111364 |
| 59170 | 124117380 | 124119552 | 111358, 111361 |
| 59171 | 124196409 | 124197990 | 185344 |
| 59172 | 124433389 | 124434674 | 119242 |
| 59173 | 124456141 | 124458882 | 178882, 179195, 119242, |
| | | | 270095 |
| 59174 | 124475883 | 124476551 | 178882, 179195 |
| 59175 | 124492623 | 124493235 | 178882, 179195 |
| 59176 | 124495920 | 124496861 | 178882, 179195 |
| 59177 | 124672875 | 124673494 | 178882 |
| 59178 | 124844210 | 124847048 | 196498 |
| 59179 | 124853438 | 124855392 | 196498 |
| 59180 | 124866139 | 124867004 | 196498 |
| 59181 | 124873290 | 124875271 | 196498 |
| 59182 | 124906815 | 124908365 | 196498 |
| 59183 | 124975236 | 124976163 | 196498 |
| 59184 | 125001619 | 125005502 | 196498 |
| 59185 | 125022391 | 125023523 | 196498 |
| 59186 | 125050862 | 125052821 | 196498 |
| 59187 | 125163920 | 125167408 | |
| 59188 | 125205253 | 125205634 | |
| 59189 | 125210947 | 125212787 | |
| 59190 | 125214878 | 125215496 | |
| 59191 | 125230184 | 125232496 | |
| 59192 | 125234575 | 125235059 | |
| 59193 | 125239634 | 125240252 | |
| 59194 | 125258344 | 125260385 | |
| 59195 | 125261647 | 125262947 | 073060 |
| 59196 | 125288672 | 125289495 | 073060 |
| 59197 | 125305575 | 125307304 | 073060 |
| 59198 | 125311918 | 125315054 | 073060 |
| 59199 | 125315798 | 125316953 | 073060 |
| 59200 | 125322366 | 125323615 | 073060 |
| 59201 | 125336390 | 125339225 | 073060 |
| 59202 | 125347136 | 125349361 | 073060 |
| 59203 | 125351297 | 125352823 | 073060 |
| 59204 | 125360047 | 125360668 | 073060 |
| 59205 | 125384541 | 125385875 | |
| 59206 | 125388963 | 125389807 | |
| 59207 | 125393641 | 125396453 | 150991 |
| 59208 | 125398072 | 125403346 | 265345, 150991 |
| 59209 | 125411613 | 125412901 | |
| 59210 | 125422053 | 125425803 | |
| 59211 | 125472876 | 125473965 | 150990 |
| 59212 | 125478215 | 125479484 | 184992 |
| 59213 | 125549437 | 125550880 | 081760 |
| 59214 | 125871351 | 125872349 | 139364 |
| 59215 | 126118001 | 126118887 | 139364 |
| 59216 | 126119366 | 126119747 | 139364 |
| 59217 | 126345852 | 126346780 | |
| 59218 | 126466071 | 126466948 | 249267 |
| 59219 | 126466994 | 126469195 | 256276, 249267 |
| 59220 | 126480943 | 126481511 | |
| 59221 | 126518175 | 126518906 | |
| 59222 | 126751892 | 126752970 | |
| 59223 | 126796711 | 126797444 | |
| 59224 | 127066700 | 127067724 | |
| 59225 | 127091973 | 127093481 | |
| 59226 | 127256489 | 127257856 | 256128 |
| 59227 | 127420889 | 127421884 | 249345 |
| 59228 | 127491367 | 127492131 | 249345 |
| 59229 | 127493389 | 127494165 | 249345 |
| 59230 | 127567335 | 127568755 | |
| 59231 | 127609618 | 127616661 | |
| 59232 | 127649856 | 127651164 | 239776 |
| 59233 | 127714307 | 127716912 | |
| 59234 | 127722667 | 127727340 | |
| 59235 | 127727992 | 127731519 | |
| 59236 | 129237305 | 129240940 | |
| 59237 | 129298081 | 129298923 | 139370 |
| 59238 | 129301697 | 129303425 | 139370 |
| 59239 | 129307292 | 129309725 | 139370 |
| 59240 | 129337535 | 129339247 | 151948 |
| 59241 | 129345226 | 129346715 | 151948 |
| 59242 | 129355431 | 129356518 | 151948 |
| 59243 | 129389368 | 129391590 | 151948 |
| 59244 | 129394051 | 129394924 | 151948 |
| 59245 | 129405019 | 129405514 | 151948 |
| 59246 | 129423371 | 129423879 | 151948 |
| 59247 | 130015501 | 130016104 | 151952 |
| 59248 | 131322725 | 131324213 | 111450 |
| 59249 | 131355392 | 131357766 | 132341 |
| 59250 | 131438264 | 131439395 | 111452 |
| 59251 | 131455992 | 131457391 | 111452 |
| 59252 | 131495894 | 131497819 | 111452 |
| 59253 | 131574726 | 131576084 | 111452 |
| 59254 | 132194544 | 132196730 | 061936 |
| 59255 | 132344619 | 132346984 | 256955 |
| 59256 | 132354457 | 132354915 | |
| 59257 | 132378436 | 132381738 | 177169 |
| 59258 | 132396829 | 132397373 | 177169 |
| 59259 | 132412868 | 132415300 | 177192, 255992 |
| 59260 | 132434039 | 132435767 | 183495 |
| 59261 | 132568352 | 132570099 | 185684 |
| 59262 | 132627712 | 132629944 | 184967, 185163 |
| 59263 | 132646963 | 132647700 | |
| 59264 | 132673828 | 132675257 | 256312, 256576 |
| 59265 | 133066972 | 133068157 | 112787 |
| 59266 | 133136260 | 133137359 | 112787 |
| 59267 | 133184236 | 133186042 | 204583 |
| 59268 | 133248690 | 133249973 | 177084 |
| 59269 | 133263261 | 133264962 | 256632, 176894, 177084 |
| 59270 | 133265511 | 133266804 | 256632, 176894 |
| 59271 | 133286352 | 133288521 | 247077, 256632, 176894 |
| 59272 | 133335898 | 133339533 | 176915 |
| 59273 | 133404306 | 133406080 | 090615, 072609 |
| 59274 | 133442798 | 133443471 | 072609 |
| 59275 | 133463305 | 133465268 | 236617, 072609 |
| 59276 | 133531552 | 133533508 | 072609, 196458 |
| 59277 | 133562494 | 133564039 | 198393 |
| 59278 | 133613537 | 133614922 | 198040, 246394 |
| 59279 | 133656480 | 133658370 | 196387 |
| 59280 | 133706009 | 133708043 | 256825, 090612, 256223, |
| | | | 214029 |
| 59281 | 133757677 | 133758944 | 256825, 090612 |

The locations of ODSCs on Chromosome 13 are shown in Table 55.

TABLE 55
ODSC Locations on Chromosome 13

| SEQ ID NO. | Start | End | Overlapping Gene(s) |
|---|---|---|---|
| 59282 | 19568043 | 19568589 | 226057 |
| 59283 | 19569173 | 19571163 | 226057 |
| 59284 | 20170967 | 20171518 | |
| 59285 | 20192993 | 20193730 | |
| 59286 | 20206816 | 20208494 | 196199 |
| 59287 | 20259026 | 20259911 | 121390 |
| 59288 | 20355765 | 20357498 | 121390 |
| 59289 | 20419843 | 20421018 | 132950 |
| 59290 | 20436157 | 20439326 | 132950 |
| 59291 | 20531412 | 20534763 | 121741 |
| 59292 | 20692288 | 20693569 | |
| 59293 | 20694082 | 20695610 | |
| 59294 | 20701488 | 20703198 | |
| 59295 | 20765607 | 20767686 | 165474 |
| 59296 | 21066639 | 21067241 | 165475 |
| 59297 | 21069093 | 21070771 | 165475 |
| 59298 | 21077170 | 21078394 | 165475 |
| 59299 | 21094046 | 21094611 | 165475 |
| 59300 | 21098734 | 21101475 | 165475 |
| 59301 | 21131208 | 21135180 | |
| 59302 | 21139512 | 21142972 | 032742 |
| 59303 | 21154448 | 21154883 | 032742 |
| 59304 | 21278021 | 21278536 | 172458 |
| 59305 | 21346859 | 21348519 | 150456 |
| 59306 | 21387629 | 21390067 | 132953 |
| 59307 | 21475775 | 21477678 | 132953 |
| 59308 | 21513894 | 21514699 | 233780 |
| 59309 | 21527492 | 21528050 | |
| 59310 | 21596327 | 21597017 | 150457 |
| 59311 | 21606680 | 21609025 | 150457 |
| 59312 | 21634110 | 21636410 | 150457 |
| 59313 | 21643653 | 21644285 | |
| 59314 | 21649599 | 21650656 | |
| 59315 | 21713733 | 21715681 | 252128, 150459 |
| 59316 | 21750384 | 21751776 | 173141, 165480 |
| 59317 | 21976144 | 21977535 | 180776 |
| 59318 | 22009565 | 22010384 | 180776 |
| 59319 | 22032458 | 22033790 | 180776 |
| 59320 | 22115061 | 22115974 | 165487 |
| 59321 | 22177482 | 22178702 | 165487 |
| 59322 | 22681551 | 22682152 | |
| 59323 | 23948794 | 23949907 | 151835 |
| 59324 | 24001275 | 24002261 | 229558, 151835 |
| 59325 | 24040098 | 24041495 | 232977 |
| 59326 | 24172755 | 24174915 | 127863 |
| 59327 | 24194716 | 24195960 | 127863 |
| 59328 | 24353046 | 24353574 | 027001 |
| 59329 | 24462727 | 24463915 | 205861, 027001 |
| 59330 | 24605041 | 24608990 | 273167, 228741, 182957 |
| 59331 | 24627635 | 24628442 | 273167, 182957 |
| 59332 | 24637990 | 24639251 | 273167, 182957 |
| 59333 | 24648570 | 24650792 | 273167, 182957 |
| 59334 | 24671169 | 24671786 | 273167, 182957 |
| 59335 | 24680675 | 24680993 | 273167, 182957 |
| 59336 | 24721281 | 24724447 | 273167, 182957 |
| 59337 | 24734042 | 24736525 | 273167, 182957 |
| 59338 | 24742442 | 24744079 | 273167, 182957 |
| 59339 | 24756385 | 24760340 | 273167, 182957 |
| 59340 | 24782460 | 24782973 | 273167, 182957 |
| 59341 | 24786422 | 24787514 | 273167, 182957 |
| 59342 | 24789686 | 24790444 | 273167, 182957 |
| 59343 | 24791557 | 24792826 | 273167, 182957 |
| 59344 | 24792980 | 24793500 | 273167, 182957 |
| 59345 | 24793660 | 24796209 | 273167, 182957 |
| 59346 | 24844418 | 24845422 | 273167, 182957 |
| 59347 | 24880623 | 24881208 | 273167, 182957 |
| 59348 | 25085497 | 25087616 | 102699, 205822 |
| 59349 | 25496556 | 25498163 | 151849 |
| 59350 | 25499651 | 25500354 | 253771 |
| 59351 | 25860677 | 25861981 | 139505 |
| 59352 | 25874768 | 25876770 | 139496, 260509 |
| 59353 | 26759698 | 26761818 | 127870 |
| 59354 | 26795090 | 26797295 | 127870 |
| 59355 | 26827535 | 26829644 | 132964 |
| 59356 | 27131609 | 27132845 | 132970 |
| 59357 | 27195854 | 27196639 | 237001, 132970 |
| 59358 | 27530534 | 27531255 | |
| 59359 | 27535955 | 27536931 | |
| 59360 | 27556479 | 27558374 | |
| 59361 | 27561582 | 27562975 | |
| 59362 | 27565111 | 27566324 | |
| 59363 | 27592585 | 27593245 | |
| 59364 | 27703826 | 27706469 | 152484 |
| 59365 | 27744472 | 27747238 | 230641, 152484 |
| 59366 | 27756282 | 27759677 | 230641 |
| 59367 | 27762110 | 27762867 | |
| 59368 | 27768382 | 27770291 | |
| 59369 | 27773778 | 27777168 | |
| 59370 | 27824574 | 27827199 | 122026 |
| 59371 | 27844557 | 27845543 | 122035 |
| 59372 | 27941557 | 27942380 | |
| 59373 | 27953898 | 27954686 | |
| 59374 | 27976801 | 27980850 | |
| 59375 | 27997916 | 28001370 | 122034 |
| 59376 | 28023222 | 28026017 | 122033 |
| 59377 | 28029233 | 28030310 | |
| 59378 | 28051199 | 28052323 | |
| 59379 | 28123203 | 28124464 | 139517 |
| 59380 | 28173908 | 28174628 | 139517 |
| 59381 | 28181155 | 28182108 | 139517 |
| 59382 | 28191103 | 28196958 | 186184, 139517 |
| 59383 | 28215741 | 28216811 | 186184 |
| 59384 | 28235062 | 28235901 | 186184 |
| 59385 | 28403955 | 28404663 | |
| 59386 | 28442731 | 28443687 | 247381 |
| 59387 | 28526862 | 28529363 | 260704 |
| 59388 | 28534185 | 28535501 | |
| 59389 | 28597292 | 28598021 | 122025 |
| 59390 | 28633283 | 28633839 | 122025 |
| 59391 | 28711515 | 28713850 | 152520, 261485 |
| 59392 | 28735196 | 28736434 | 152520 |
| 59393 | 29212028 | 29212456 | |
| 59394 | 29232285 | 29234635 | 132963 |
| 59395 | 29291681 | 29293866 | 139508 |
| 59396 | 29303664 | 29304008 | |
| 59397 | 29321918 | 29322569 | |
| 59398 | 29327342 | 29328585 | |
| 59399 | 29393259 | 29395039 | |
| 59400 | 29417699 | 29418255 | |
| 59401 | 29557637 | 29559628 | |
| 59402 | 29697529 | 29698547 | 132938 |
| 59403 | 29709474 | 29711896 | 132938 |
| 59404 | 29713903 | 29715270 | 132938 |
| 59405 | 30168305 | 30170110 | 139514 |
| 59406 | 30250706 | 30251170 | |
| 59407 | 30421507 | 30422483 | 122042 |
| 59408 | 30422576 | 30425192 | 122042 |
| 59409 | 30479877 | 30480341 | |
| 59410 | 30492302 | 30495976 | 224405 |
| 59411 | 30496116 | 30500118 | 224405 |
| 59412 | 30514896 | 30515296 | 122043 |
| 59413 | 30528529 | 30529661 | |
| 59414 | 30682089 | 30683835 | 224511 |
| 59415 | 30686785 | 30690173 | |
| 59416 | 30699724 | 30700176 | |
| 59417 | 30715724 | 30717937 | |
| 59418 | 30880460 | 30882065 | 102781 |
| 59419 | 30908339 | 30909746 | |
| 59420 | 30912348 | 30913729 | |
| 59421 | 30913865 | 30917252 | 238121 |
| 59422 | 30945017 | 30946835 | 238121 |
| 59423 | 30957029 | 30957766 | |
| 59424 | 30962839 | 30964394 | |
| 59425 | 30968629 | 30971134 | |
| 59426 | 30972391 | 30973906 | |
| 59427 | 30977739 | 30979137 | |
| 59428 | 30982279 | 30983558 | |
| 59429 | 30995141 | 30997167 | 225039 |
| 59430 | 31003545 | 31006468 | |
| 59431 | 31015387 | 31015917 | |
| 59432 | 31037441 | 31040496 | 189403 |
| 59433 | 31146424 | 31147509 | 189403 |
| 59434 | 31150472 | 31151993 | 189403 |
| 59435 | 31189880 | 31194695 | 132952, 189403 |
| 59436 | 31247528 | 31249065 | |
| 59437 | 31366643 | 31367337 | |
| 59438 | 31376908 | 31378014 | 237879 |
| 59439 | 31734666 | 31737630 | 120694 |
| 59440 | 31746342 | 31747333 | |
| 59441 | 31773655 | 31775870 | 187676 |
| 59442 | 32280228 | 32280763 | |
| 59443 | 32500515 | 32501779 | 229715 |
| 59444 | 32501932 | 32502557 | 229715 |
| 59445 | 32519382 | 32520419 | 229715 |
| 59446 | 32556400 | 32557125 | |
| 59447 | 32605186 | 32606242 | 073910, 237637 |
| 59448 | 32716891 | 32717510 | 073910 |
| 59449 | 32827871 | 32831061 | 073910 |
| 59450 | 32834913 | 32836037 | 073910 |
| 59451 | 32872511 | 32873467 | 230633 |
| 59452 | 32888885 | 32890759 | 139618, 189167 |
| 59453 | 32983113 | 32984737 | 139597 |
| 59454 | 33000220 | 33003832 | 139597, 270008 |
| 59455 | 33027242 | 33028449 | 244754 |
| 59456 | 33089272 | 33089878 | 244754 |
| 59457 | 33111716 | 33113929 | 244754 |
| 59458 | 33159698 | 33162033 | 083642 |
| 59459 | 33412920 | 33413602 | |
| 59460 | 33543350 | 33544315 | |
| 59461 | 33627528 | 33628504 | 133116 |
| 59462 | 33698954 | 33701903 | 133121 |
| 59463 | 33702876 | 33703611 | 133121 |
| 59464 | 33808707 | 33810860 | 133121 |
| 59465 | 33824504 | 33826082 | 133121 |
| 59466 | 33832661 | 33833376 | 133121 |
| 59467 | 33834424 | 33839269 | 133121 |
| 59468 | 33845199 | 33849643 | 236581, 133121 |
| 59469 | 33851966 | 33861039 | 236581, 133121 |
| 59470 | 34067665 | 34069490 | 230490 |
| 59471 | 34098172 | 34098993 | 230490 |
| 59472 | 34115768 | 34118701 | 230490 |
| 59473 | 34134655 | 34135304 | 230490 |
| 59474 | 34184245 | 34186297 | 234535, 230490 |
| 59475 | 34202760 | 34203471 | 230490 |
| 59476 | 34212133 | 34218431 | 230490 |
| 59477 | 34249941 | 34251240 | 230490 |
| 59478 | 34252499 | 34254621 | |
| 59479 | 34296903 | 34298462 | |
| 59480 | 34302390 | 34303308 | |
| 59481 | 34391532 | 34392903 | 133119 |
| 59482 | 34444310 | 34444846 | 133119 |
| 59483 | 34445427 | 34446485 | 133119 |
| 59484 | 34492470 | 34493232 | 133119 |
| 59485 | 34542703 | 34543961 | |
| 59486 | 34585608 | 34586375 | |
| 59487 | 34593683 | 34594890 | |
| 59488 | 34775359 | 34776729 | |
| 59489 | 34781247 | 34782997 | |
| 59490 | 35923822 | 35924666 | 172915 |
| 59491 | 36500780 | 36502861 | 133083 |
| 59492 | 36919077 | 36921229 | 120664, 133104 |
| 59493 | 37393167 | 37394149 | 133111 |
| 59494 | 37572527 | 37576240 | 120699, 120697 |
| 59495 | 37632858 | 37634211 | 102710 |
| 59496 | 38922355 | 38925041 | 120686 |
| 59497 | 39337745 | 39339073 | 150893 |
| 59498 | 39422320 | 39423565 | 150893 |
| 59499 | 39452900 | 39454306 | 150893 |
| 59500 | 39611165 | 39613713 | 188811, 120685 |
| 59501 | 40176859 | 40178179 | 183722 |
| 59502 | 40229304 | 40230643 | 133103 |
| 59503 | 40637928 | 40638910 | |
| 59504 | 40755450 | 40756089 | 230710 |
| 59505 | 40795360 | 40795912 | |
| 59506 | 40810109 | 40810993 | |
| 59507 | 40910103 | 40911647 | |
| 59508 | 40917859 | 40919496 | 230058 |
| 59509 | 40919552 | 40921030 | 230058 |
| 59510 | 40976141 | 40977363 | |
| 59511 | 41015162 | 41016126 | |
| 59512 | 41050315 | 41051320 | 215483 |
| 59513 | 41051614 | 41053368 | 215483 |
| 59514 | 41076115 | 41079754 | |
| 59515 | 41082110 | 41082935 | |
| 59516 | 41091680 | 41093962 | |
| 59517 | 41110497 | 41111993 | 269120 |
| 59518 | 41114251 | 41115249 | |
| 59519 | 41137453 | 41137850 | 150907 |
| 59520 | 41138167 | 41139191 | 150907 |
| 59521 | 41225940 | 41226482 | 150907 |
| 59522 | 41231637 | 41232825 | 150907 |
| 59523 | 41234116 | 41234797 | 150907 |
| 59524 | 41236723 | 41241887 | 150907 |
| 59525 | 41344512 | 41346175 | 102738 |
| 59526 | 41360225 | 41361482 | |
| 59527 | 41362637 | 41365595 | 102743 |
| 59528 | 41368332 | 41369863 | 102743 |
| 59529 | 41372386 | 41373626 | 102743 |
| 59530 | 41375368 | 41376840 | 102743 |
| 59531 | 41581860 | 41583058 | 120690 |
| 59532 | 41591272 | 41594076 | 120690 |
| 59533 | 41598993 | 41600552 | 120690 |
| 59534 | 41626281 | 41628893 | 120690 |
| 59535 | 41633092 | 41637217 | 120688, 120690 |
| 59536 | 41638702 | 41639765 | 120688 |
| 59537 | 41706481 | 41707356 | 165572 |
| 59538 | 41767973 | 41769012 | 120696 |
| 59539 | 41793070 | 41793994 | 120662 |
| 59540 | 41837097 | 41838180 | 120662 |
| 59541 | 41884919 | 41887008 | 172766 |
| 59542 | 42151464 | 42152190 | 102763 |
| 59543 | 42162240 | 42165094 | 102763 |
| 59544 | 42180746 | 42181229 | 102763 |
| 59545 | 42196201 | 42197101 | 102763 |
| 59546 | 42213859 | 42214758 | 102763 |
| 59547 | 42219479 | 42219978 | 102763 |
| 59548 | 42268116 | 42271981 | 102763 |
| 59549 | 42293442 | 42296037 | 102763 |
| 59550 | 42376532 | 42377958 | 102763 |
| 59551 | 42401799 | 42403027 | 102763 |
| 59552 | 42516829 | 42517863 | 102763 |
| 59553 | 42529707 | 42530264 | 102763 |
| 59554 | 42533373 | 42536746 | 102763 |
| 59555 | 42556088 | 42556947 | |
| 59556 | 42601852 | 42603373 | |
| 59557 | 42613608 | 42616341 | 102780 |
| 59558 | 42621529 | 42623747 | 102780 |
| 59559 | 42679196 | 42680842 | 102780 |
| 59560 | 42845621 | 42847309 | 023516 |
| 59561 | 42893958 | 42894972 | 023516 |
| 59562 | 42920938 | 42921401 | |
| 59563 | 43061103 | 43061756 | |
| 59564 | 43097445 | 43100875 | |
| 59565 | 43127265 | 43128503 | |
| 59566 | 43168689 | 43169602 | 120659 |
| 59567 | 43406455 | 43407338 | |
| 59568 | 43559477 | 43560407 | 133106 |
| 59569 | 43565560 | 43567418 | 133106 |
| 59570 | 43597007 | 43598558 | 120675 |
| 59571 | 43628711 | 43630150 | 120675 |
| 59572 | 43661912 | 43663018 | 120675 |
| 59573 | 43843151 | 43843868 | 120658 |
| 59574 | 43881916 | 43882611 | 120658 |
| 59575 | 43894155 | 43894710 | 120658 |
| 59576 | 44422363 | 44422847 | 151773 |
| 59577 | 44452511 | 44454849 | 179630, 151773 |
| 59578 | 44677896 | 44678511 | |
| 59579 | 44681461 | 44683854 | 226519 |
| 59580 | 44714971 | 44718709 | 139656, 227258 |
| 59581 | 44765446 | 44766305 | 227258 |
| 59582 | 44792730 | 44793502 | 227258 |
| 59583 | 44794100 | 44794837 | 227258 |
| 59584 | 44805514 | 44806737 | 227258 |
| 59585 | 44855593 | 44856241 | |
| 59586 | 44873188 | 44877164 | |
| 59587 | 44879762 | 44881567 | |
| 59588 | 44885982 | 44888815 | |
| 59589 | 44896982 | 44897689 | |
| 59590 | 44938024 | 44939427 | |
| 59591 | 44947198 | 44948906 | 151778 |
| 59592 | 44970955 | 44971929 | 151778 |
| 59593 | 44977618 | 44980207 | 237361 |
| 59594 | 45007951 | 45012801 | 225727, 102804 |
| 59595 | 45022246 | 45023226 | 225727, 102804 |
| 59596 | 45024272 | 45024946 | 225727, 102804 |
| 59597 | 45097212 | 45097879 | 102804 |
| 59598 | 45150787 | 45152731 | 102804 |
| 59599 | 45389600 | 45390169 | |
| 59600 | 45491527 | 45493558 | |
| 59601 | 45562757 | 45564539 | 133114, 083635, 223732 |
| 59602 | 45694127 | 45695959 | 188342 |
| 59603 | 45742349 | 45742775 | 188342 |
| 59604 | 45780175 | 45782419 | 188342 |
| 59605 | 45792681 | 45795083 | 188342 |
| 59606 | 45810141 | 45810608 | 188342 |
| 59607 | 45890167 | 45891238 | |
| 59608 | 45909659 | 45910341 | |
| 59609 | 45914403 | 45916804 | 170919, 273149, 133112 |
| 59610 | 45988414 | 45989528 | 174032 |
| 59611 | 45991009 | 45993083 | 251015, 174032 |
| 59612 | 46006871 | 46008266 | |
| 59613 | 46014297 | 46016627 | |
| 59614 | 46021124 | 46021980 | |
| 59615 | 46038102 | 46040039 | 136152 |
| 59616 | 46101290 | 46102452 | 136152 |
| 59617 | 46381499 | 46382111 | 215475 |
| 59618 | 46605856 | 46606486 | 123200 |
| 59619 | 46626133 | 46627255 | 235903, 123200 |
| 59620 | 46670952 | 46672923 | 080618, 235903 |
| 59621 | 46674228 | 46676675 | 080618, 235903 |
| 59622 | 46678440 | 46679562 | 080618, 235903 |
| 59623 | 46694319 | 46694783 | |
| 59624 | 46695824 | 46698060 | |
| 59625 | 46700458 | 46702161 | 136167 |
| 59626 | 46716738 | 46717895 | 136167 |
| 59627 | 46869207 | 46871959 | 261097 |
| 59628 | 47126422 | 47129578 | 136141 |
| 59629 | 47149744 | 47151026 | 136141 |
| 59630 | 47174129 | 47175680 | 136141 |
| 59631 | 47180876 | 47182694 | 136141 |
| 59632 | 47207279 | 47207993 | 136141 |
| 59633 | 47229774 | 47231278 | 136141 |
| 59634 | 47233558 | 47234244 | 136141 |
| 59635 | 47234444 | 47235515 | 136141 |
| 59636 | 47249330 | 47250743 | 136141 |
| 59637 | 47253285 | 47254634 | 136141 |
| 59638 | 47258642 | 47259147 | 136141 |
| 59639 | 47261669 | 47262569 | 136141 |
| 59640 | 47333716 | 47334643 | |
| 59641 | 47369916 | 47373641 | 139684 |
| 59642 | 47420620 | 47421344 | 102468 |
| 59643 | 47468134 | 47468686 | 102468 |
| 59644 | 47789674 | 47790987 | |
| 59645 | 48564686 | 48565671 | 136143 |
| 59646 | 48574595 | 48576219 | 227848, 136143 |
| 59647 | 48583277 | 48584006 | 136143 |
| 59648 | 48611475 | 48612893 | 136159, 136143 |
| 59649 | 48668314 | 48669583 | 136146 |
| 59650 | 48775120 | 48775565 | |
| 59651 | 48796460 | 48797357 | |
| 59652 | 48806682 | 48810247 | 136156 |
| 59653 | 48836319 | 48838395 | 136156 |
| 59654 | 48876860 | 48879058 | 139687, 231473 |
| 59655 | 49105811 | 49107824 | 136161 |
| 59656 | 49121815 | 49122341 | |
| 59657 | 49322681 | 49324887 | |
| 59658 | 49356731 | 49357877 | |
| 59659 | 49382468 | 49383302 | |
| 59660 | 49476491 | 49477110 | |
| 59661 | 49482558 | 49483419 | |
| 59662 | 49549050 | 49552332 | 102531 |
| 59663 | 49671194 | 49673869 | 102531 |
| 59664 | 49683917 | 49685446 | 102531 |
| 59665 | 49788604 | 49789727 | |
| 59666 | 49821055 | 49823044 | 102543 |
| 59667 | 49887473 | 49889148 | 102547 |
| 59668 | 49900032 | 49901440 | 102547 |
| 59669 | 49930016 | 49931132 | 102547 |
| 59670 | 50017175 | 50020042 | 136169, 102547, 215462 |
| 59671 | 50039181 | 50039586 | 136169 |
| 59672 | 50040154 | 50041223 | 136169 |
| 59673 | 50069431 | 50071141 | 136147 |
| 59674 | 50158681 | 50162452 | 136144 |
| 59675 | 50198921 | 50200738 | |
| 59676 | 50264215 | 50266485 | 123179 |
| 59677 | 50285454 | 50286023 | 102753 |
| 59678 | 50307542 | 50308140 | 102753 |
| 59679 | 50365126 | 50367503 | 102753 |
| 59680 | 50384072 | 50386570 | |
| 59681 | 50431059 | 50435439 | |
| 59682 | 50509342 | 50511411 | 123178 |
| 59683 | 50512680 | 50514002 | |
| 59684 | 50569929 | 50572415 | 204977, 264864 |
| 59685 | 50654498 | 50657391 | 176124, 231607 |
| 59686 | 50697117 | 50700917 | 231607, 176124 |
| 59687 | 50734977 | 50735758 | 176124 |
| 59688 | 50796429 | 50797816 | 176124 |
| 59689 | 50804008 | 50805029 | 176124 |
| 59690 | 50818196 | 50820428 | 176124 |
| 59691 | 50841888 | 50842615 | 176124 |
| 59692 | 50893472 | 50895461 | 176124 |
| 59693 | 50897696 | 50898220 | 176124 |
| 59694 | 50931000 | 50933336 | 176124 |
| 59695 | 50939659 | 50941100 | 176124 |
| 59696 | 50960565 | 50961911 | 176124 |
| 59697 | 50964799 | 50965553 | 176124 |
| 59698 | 51086326 | 51088041 | 176124 |
| 59699 | 51092331 | 51093390 | 176124 |
| 59700 | 51095693 | 51096503 | 229323, 176124 |
| 59701 | 51101937 | 51105300 | 176124 |
| 59702 | 51483530 | 51485482 | 136104, 233672 |
| 59703 | 51708643 | 51709332 | 226792 |
| 59704 | 51725383 | 51726068 | 226792 |
| 59705 | 52024627 | 52029150 | 236778, 224892, 102786 |
| 59706 | 52050098 | 52054200 | 236778 |
| 59707 | 52092272 | 52096266 | 236778 |
| 59708 | 52096782 | 52100883 | 236778 |
| 59709 | 52157542 | 52159839 | 139668 |
| 59710 | 52164487 | 52165540 | 139668 |
| 59711 | 52210233 | 52211443 | 139668 |
| 59712 | 52217180 | 52218633 | 139668 |
| 59713 | 52242554 | 52243846 | 139668 |
| 59714 | 52300224 | 52303530 | 139668 |
| 59715 | 52303564 | 52305597 | 139668 |
| 59716 | 52338135 | 52339036 | |
| 59717 | 52356339 | 52357008 | 102796 |
| 59718 | 52367029 | 52368697 | 102796 |
| 59719 | 52375438 | 52379259 | 231856, 102796 |
| 59720 | 52388947 | 52389901 | |
| 59721 | 52419122 | 52420115 | |
| 59722 | 52552955 | 52553571 | 123191 |
| 59723 | 52569585 | 52570331 | 123191 |
| 59724 | 52571537 | 52572651 | 123191 |
| 59725 | 52585088 | 52587272 | 253710, 123191 |
| 59726 | 52599257 | 52600273 | 253710, 253797 |
| 59727 | 52701422 | 52703642 | 197168 |
| 59728 | 52732564 | 52734508 | 136098 |
| 59729 | 52768296 | 52769994 | 243406, 217576 |
| 59730 | 52979620 | 52980719 | 136114 |
| 59731 | 52985651 | 52986770 | 136100 |
| 59732 | 52988541 | 52989863 | 136100 |
| 59733 | 53023508 | 53025192 | 136100 |
| 59734 | 53029033 | 53030271 | 136108 |
| 59735 | 53191024 | 53191947 | 250299, 139675 |
| 59736 | 53225556 | 53227869 | 165416 |
| 59737 | 53405001 | 53405427 | |
| 59738 | 53542146 | 53542654 | |
| 59739 | 57619855 | 57620980 | |
| 59740 | 57697531 | 57698193 | |
| 59741 | 57995398 | 57995874 | |
| 59742 | 58198776 | 58199431 | |
| 59743 | 60265006 | 60266128 | 139734 |
| 59744 | 60738008 | 60739209 | 139734 |
| 59745 | 60969867 | 60972592 | 083544 |
| 59746 | 61146509 | 61147097 | 083544 |
| 59747 | 61989075 | 61989793 | 197991 |
| 59748 | 62239426 | 62240042 | |
| 59749 | 62369652 | 62370141 | |
| 59750 | 67483845 | 67485156 | 184226, 228842 |
| 59751 | 67567548 | 67569807 | 233840, 184226 |
| 59752 | 67650518 | 67652020 | 184226 |
| 59753 | 67657928 | 67658867 | 184226 |
| 59754 | 67675541 | 67677406 | 184226 |
| 59755 | 67692070 | 67696646 | 184226 |
| 59756 | 67711153 | 67715219 | 184226 |
| 59757 | 67721417 | 67723000 | 184226 |
| 59758 | 67795786 | 67798873 | 184226 |
| 59759 | 67804384 | 67805726 | 184226 |
| 59760 | 67957492 | 67957961 | |
| 59761 | 68620789 | 68621450 | |
| 59762 | 68631145 | 68631716 | |
| 59763 | 70965611 | 70966328 | |
| 59764 | 71031425 | 71032084 | |
| 59765 | 71864454 | 71866355 | |
| 59766 | 73301095 | 73301755 | 204899 |
| 59767 | 73354962 | 73357148 | 083535, 083520 |
| 59768 | 73366086 | 73367280 | 083535 |
| 59769 | 73425145 | 73425898 | 220981, 083535 |
| 59770 | 73632644 | 73634922 | 102554 |
| 59771 | 73664033 | 73665353 | |
| 59772 | 73768313 | 73769427 | |
| 59773 | 73910764 | 73912085 | |
| 59774 | 73912502 | 73912990 | |
| 59775 | 74181926 | 74182710 | 224853 |
| 59776 | 74246459 | 74247190 | |
| 59777 | 74339435 | 74340891 | 118922 |
| 59778 | 74383643 | 74384108 | 118922 |
| 59779 | 74546511 | 74547153 | 118922 |
| 59780 | 74648867 | 74649334 | 118922 |
| 59781 | 74702143 | 74702794 | 118922 |
| 59782 | 74706351 | 74710885 | 118922 |
| 59783 | 74727317 | 74727767 | |
| 59784 | 74736296 | 74737144 | |
| 59785 | 74742075 | 74743007 | |
| 59786 | 74761363 | 74762352 | |
| 59787 | 74795795 | 74796748 | |
| 59788 | 74801288 | 74802465 | |
| 59789 | 74860743 | 74863151 | |
| 59790 | 75739649 | 75740390 | |
| 59791 | 75903790 | 75904308 | 136111 |
| 59792 | 75913579 | 75914574 | 136111 |
| 59793 | 75989477 | 75990540 | 136111 |
| 59794 | 76014122 | 76014987 | 136111 |
| 59795 | 76026504 | 76027279 | 136111 |
| 59796 | 76032810 | 76033751 | 136111 |
| 59797 | 76054775 | 76057130 | 136111 |
| 59798 | 76063888 | 76064542 | |
| 59799 | 76110954 | 76112642 | 188243 |
| 59800 | 76118913 | 76119608 | 188243 |
| 59801 | 76123277 | 76124738 | 118939, 261553, 188243 |
| 59802 | 76209848 | 76210807 | 261105, 136153, 261553 |
| 59803 | 76334300 | 76337573 | 136153, 261553 |
| 59804 | 76344202 | 76348625 | 136153, 261553 |
| 59805 | 76366523 | 76367745 | 136153, 261553 |
| 59806 | 76368717 | 76369240 | 136153, 261553 |
| 59807 | 76410182 | 76411065 | 136153 |
| 59808 | 77565424 | 77567397 | 005812, 102805 |
| 59809 | 77600193 | 77602216 | 005812 |
| 59810 | 77717355 | 77718108 | 005810 |
| 59811 | 77899614 | 77901682 | 005810 |
| 59812 | 77902875 | 77904612 | |
| 59813 | 77930208 | 77930845 | |
| 59814 | 77960946 | 77961929 | |
| 59815 | 78051914 | 78053017 | |
| 59816 | 78266732 | 78267421 | |
| 59817 | 78271378 | 78273311 | 139737, 266325 |
| 59818 | 79010213 | 79010948 | 234377 |
| 59819 | 79232380 | 79233608 | 152193 |
| 59820 | 79483945 | 79484982 | 230584 |
| 59821 | 79856574 | 79857671 | |
| 59822 | 79979066 | 79981522 | 227354, 139746 |
| 59823 | 80054644 | 80056583 | 102471, 232132 |
| 59824 | 80059058 | 80059896 | 102471 |
| 59825 | 80108101 | 80108618 | 102471 |
| 59826 | 80411574 | 80412458 | |
| 59827 | 80604482 | 80605252 | |
| 59828 | 80648186 | 80649531 | |
| 59829 | 80706734 | 80707667 | |
| 59830 | 80833175 | 80833784 | |
| 59831 | 80910473 | 80910849 | 136158 |
| 59832 | 80912223 | 80917718 | 136158 |
| 59833 | 80946115 | 80946723 | |
| 59834 | 80953088 | 80954020 | |
| 59835 | 81018297 | 81019323 | |
| 59836 | 81037671 | 81038333 | |
| 59837 | 81042561 | 81046406 | |
| 59838 | 81131530 | 81132975 | |
| 59839 | 82865865 | 82866538 | |
| 59840 | 90052848 | 90056452 | |
| 59841 | 90165490 | 90168754 | |
| 59842 | 91999570 | 92002132 | 215417 |
| 59843 | 92013681 | 92014753 | |
| 59844 | 92787274 | 92788431 | 179399 |
| 59845 | 93821335 | 93823393 | |
| 59846 | 93876475 | 93883343 | 183098 |
| 59847 | 93883794 | 93885145 | 183098 |
| 59848 | 93885918 | 93886920 | 183098 |
| 59849 | 93900493 | 93902998 | 183098 |
| 59850 | 93912673 | 93913565 | 183098 |
| 59851 | 93981005 | 93981804 | 183098 |
| 59852 | 94118564 | 94119603 | 183098 |
| 59853 | 94187360 | 94187939 | 183098 |
| 59854 | 94254972 | 94256325 | 183098 |
| 59855 | 94410225 | 94411909 | 183098 |
| 59856 | 94418979 | 94421036 | 183098 |
| 59857 | 94427177 | 94429676 | 183098 |
| 59858 | 94498904 | 94499892 | 183098 |
| 59859 | 94608175 | 94609485 | 183098 |
| 59860 | 94615357 | 94617091 | 183098 |
| 59861 | 94764411 | 94764995 | 183098 |
| 59862 | 94771468 | 94772329 | 183098 |
| 59863 | 94780153 | 94781979 | 183098 |
| 59864 | 94806624 | 94807087 | 236520, 183098 |
| 59865 | 94809764 | 94810614 | 236520, 183098 |
| 59866 | 94901762 | 94902389 | 183098 |
| 59867 | 94943050 | 94945878 | 183098 |
| 59868 | 94955573 | 94958728 | 183098 |
| 59869 | 94962204 | 94963676 | 183098 |
| 59870 | 94981221 | 94983066 | 183098 |
| 59871 | 94994689 | 94995922 | 183098 |
| 59872 | 94996291 | 94998389 | 183098 |
| 59873 | 95200636 | 95202729 | |
| 59874 | 95237944 | 95238476 | 088451 |
| 59875 | 95246996 | 95248844 | 088451 |
| 59876 | 95251944 | 95252595 | |
| 59877 | 95253709 | 95254881 | 152749 |
| 59878 | 95432660 | 95433281 | |
| 59879 | 95775607 | 95776819 | 125257 |
| 59880 | 95948601 | 95949339 | 125257 |
| 59881 | 95949615 | 95950415 | 125257 |
| 59882 | 95952856 | 95954301 | 125257 |
| 59883 | 95966601 | 95967290 | |
| 59884 | 96030828 | 96031188 | |
| 59885 | 96187188 | 96188447 | 134873 |
| 59886 | 96255796 | 96257026 | 134874 |
| 59887 | 96327809 | 96330695 | 102580, 247400 |
| 59888 | 96366442 | 96367787 | 251901, 102580 |
| 59889 | 96704904 | 96706275 | 102595 |
| 59890 | 97761868 | 97762737 | |
| 59891 | 97793509 | 97794348 | |
| 59892 | 97794598 | 97795303 | |
| 59893 | 97874187 | 97878002 | 139793 |
| 59894 | 97881664 | 97883621 | 139793 |
| 59895 | 97901238 | 97901898 | 139793 |
| 59896 | 97906686 | 97914778 | 139793 |
| 59897 | 97926315 | 97929733 | 139793 |
| 59898 | 97940279 | 97941337 | 139793 |
| 59899 | 97941909 | 97942502 | 139793 |
| 59900 | 97948155 | 97949941 | 139793 |
| 59901 | 97958880 | 97961888 | 139793 |
| 59902 | 97980320 | 97980814 | 139793 |
| 59903 | 98021256 | 98022296 | 139793 |
| 59904 | 98084884 | 98086879 | 125249 |
| 59905 | 98627433 | 98630117 | 065150 |
| 59906 | 98678440 | 98679690 | |
| 59907 | 98793949 | 98797721 | 152767 |
| 59908 | 98813514 | 98814198 | 152767 |
| 59909 | 98823449 | 98827154 | 152767 |
| 59910 | 98841982 | 98843767 | 152767 |
| 59911 | 98847172 | 98847892 | 152767 |
| 59912 | 98854037 | 98855027 | 152767 |
| 59913 | 98858490 | 98860358 | 152767 |
| 59914 | 98880714 | 98881703 | 152767 |
| 59915 | 98905263 | 98905850 | 152767 |
| 59916 | 98907689 | 98908484 | 152767 |
| 59917 | 98918157 | 98918995 | 152767 |
| 59918 | 98927541 | 98928500 | 152767 |
| 59919 | 98945275 | 98946471 | 152767 |
| 59920 | 98960237 | 98960836 | 152767 |
| 59921 | 98976301 | 98976818 | 152767 |
| 59922 | 99106499 | 99107487 | 102572 |
| 59923 | 99118750 | 99120385 | 102572 |
| 59924 | 99125311 | 99126717 | 102572 |
| 59925 | 99128118 | 99129913 | 102572 |
| 59926 | 99134634 | 99137632 | 102572 |
| 59927 | 99155204 | 99156667 | 102572 |
| 59928 | 99158331 | 99159960 | 102572 |
| 59929 | 99187620 | 99188583 | 102572 |
| 59930 | 99221326 | 99222513 | 102572 |
| 59931 | 99227493 | 99230910 | 224418, 102572 |
| 59932 | 99251526 | 99252374 | |
| 59933 | 99305429 | 99306092 | |
| 59934 | 99316153 | 99318988 | |
| 59935 | 99324590 | 99326664 | |
| 59936 | 99398859 | 99399920 | 088386 |
| 59937 | 99403656 | 99405568 | 088386 |
| 59938 | 99407910 | 99408442 | |
| 59939 | 99412955 | 99414450 | |
| 59940 | 99427890 | 99428795 | |
| 59941 | 99454764 | 99455277 | 088387 |
| 59942 | 99461473 | 99462068 | 088387 |
| 59943 | 99473135 | 99476106 | 088387 |
| 59944 | 99476267 | 99477077 | 088387 |
| 59945 | 99479792 | 99482745 | 088387 |
| 59946 | 99636304 | 99636867 | 088387 |
| 59947 | 99683091 | 99684876 | 088387 |
| 59948 | 99685971 | 99687013 | 088387 |
| 59949 | 99718820 | 99720822 | 088387 |
| 59950 | 99737829 | 99740680 | 260992, 088387 |
| 59951 | 99852030 | 99854723 | 134882, 228889 |
| 59952 | 99933984 | 99934491 | 134882 |
| 59953 | 99937925 | 99938477 | 134882 |
| 59954 | 99991952 | 99992657 | 134882 |
| 59955 | 100119888 | 100120446 | |
| 59956 | 100150182 | 100151993 | 203441 |
| 59957 | 100152854 | 100154837 | 125304, 203441 |
| 59958 | 100178743 | 100179176 | 125304 |
| 59959 | 100230246 | 100231359 | 234168 |
| 59960 | 100257761 | 100259846 | 125246 |
| 59961 | 100302760 | 100303723 | 125246 |
| 59962 | 100306694 | 100309132 | 125246 |
| 59963 | 100309860 | 100311521 | 125246 |
| 59964 | 100403760 | 100404523 | 125246 |
| 59965 | 100408375 | 100409067 | 125246 |
| 59966 | 100461899 | 100466388 | 125246 |
| 59967 | 100560272 | 100561258 | |
| 59968 | 100632424 | 100633834 | |
| 59969 | 100634130 | 100634732 | 043355 |
| 59970 | 100740711 | 100742865 | 175198 |
| 59971 | 100750375 | 100753818 | 175198 |
| 59972 | 100764363 | 100765037 | 175198 |
| 59973 | 100849741 | 100850743 | 270632, 175198 |
| 59974 | 100882338 | 100883065 | 175198 |
| 59975 | 101033860 | 101034603 | 175198 |
| 59976 | 101044074 | 101046160 | 175198 |
| 59977 | 101086142 | 101086907 | 175198 |
| 59978 | 101088234 | 101088836 | 175198 |
| 59979 | 101092353 | 101093959 | 175198 |
| 59980 | 101095310 | 101096126 | 175198 |
| 59981 | 101168835 | 101169940 | 175198 |
| 59982 | 101175626 | 101176712 | 175198 |
| 59983 | 101177305 | 101178272 | 175198 |
| 59984 | 101185700 | 101186298 | 134864 |
| 59985 | 101196058 | 101197911 | 224356, 134864 |
| 59986 | 101213995 | 101214507 | 224356, 134864 |
| 59987 | 101240235 | 101241422 | 134864 |
| 59988 | 101326027 | 101328088 | 125247 |
| 59989 | 101386234 | 101387075 | 233009 |
| 59990 | 101527325 | 101529040 | 233009 |
| 59991 | 101677902 | 101680256 | 233009 |
| 59992 | 102166788 | 102167605 | 198542 |
| 59993 | 102222100 | 102223418 | 198542 |
| 59994 | 102242462 | 102243412 | 198542 |
| 59995 | 102278768 | 102280512 | 198542 |
| 59996 | 102300738 | 102303026 | 198542 |
| 59997 | 102359005 | 102359745 | 198542 |
| 59998 | 102390480 | 102391154 | 102466 |
| 59999 | 102391427 | 102393108 | 102466 |
| 60000 | 102398819 | 102399951 | 102466 |
| 60001 | 102425367 | 102425921 | 102466 |
| 60002 | 102527978 | 102529093 | 102466 |
| 60003 | 102788803 | 102790078 | 102466 |
| 60004 | 102835348 | 102837320 | 102466 |
| 60005 | 102842573 | 102846106 | 102466 |
| 60006 | 102850452 | 102851889 | 102466 |
| 60007 | 102885826 | 102886538 | 102466 |
| 60008 | 102887724 | 102889299 | 102466 |
| 60009 | 102937402 | 102939646 | 102466 |
| 60010 | 102943620 | 102944735 | 243319, 102466 |
| 60011 | 102946933 | 102948474 | 243319, 102466 |
| 60012 | 103046041 | 103048071 | 272143, 243319, 102466 |
| 60013 | 103052166 | 103053825 | 270581, 102466 |
| 60014 | 103244215 | 103245056 | |
| 60015 | 103247996 | 103250471 | 134900 |
| 60016 | 103377131 | 103377751 | |
| 60017 | 103422692 | 103423326 | 151287 |
| 60018 | 103424752 | 103426999 | 151287 |
| 60019 | 103441028 | 103441660 | 134901 |
| 60020 | 103450809 | 103453582 | 134897, 134901 |
| 60021 | 103482852 | 103483480 | 270181, 134897 |
| 60022 | 103491543 | 103492165 | 270181, 134897 |
| 60023 | 103497395 | 103500390 | 270181, 134899 |
| 60024 | 103515783 | 103516382 | 270181, 134899 |
| 60025 | 103529258 | 103530178 | |
| 60026 | 103549697 | 103550821 | |
| 60027 | 103552459 | 103553156 | |
| 60028 | 103554996 | 103555615 | |
| 60029 | 103560094 | 103561235 | |
| 60030 | 104169566 | 104170430 | |
| 60031 | 104183535 | 104184173 | |
| 60032 | 104316214 | 104316783 | |
| 60033 | 104383679 | 104384715 | |
| 60034 | 104535121 | 104536793 | |
| 60035 | 104967608 | 104969080 | |
| 60036 | 105605820 | 105607100 | |
| 60037 | 107187980 | 107188554 | |
| 60038 | 107219011 | 107220861 | 134884 |
| 60039 | 107569391 | 107570825 | |
| 60040 | 107638230 | 107640175 | |
| 60041 | 108806362 | 108808381 | |
| 60042 | 108869815 | 108871775 | 139826, 174405 |
| 60043 | 108956415 | 108957298 | 102524 |
| 60044 | 109566636 | 109570299 | 041515 |
| 60045 | 109597003 | 109597713 | 041515 |
| 60046 | 109609748 | 109610494 | 041515 |
| 60047 | 109613691 | 109614526 | 041515 |
| 60048 | 109632314 | 109633130 | 041515 |
| 60049 | 109895585 | 109896754 | |
| 60050 | 109908562 | 109911558 | |
| 60051 | 109926360 | 109927470 | |
| 60052 | 109961762 | 109962955 | 236053 |
| 60053 | 110041254 | 110043301 | |
| 60054 | 110045290 | 110045823 | |
| 60055 | 110047487 | 110050118 | |
| 60056 | 110052563 | 110053557 | 229792 |
| 60057 | 110055308 | 110055838 | |
| 60058 | 110060565 | 110061998 | |
| 60059 | 110238999 | 110239936 | |
| 60060 | 110245046 | 110245965 | |
| 60061 | 110246780 | 110247396 | |
| 60062 | 110337053 | 110338768 | |
| 60063 | 110339639 | 110341055 | |
| 60064 | 110356592 | 110358480 | |
| 60065 | 110367483 | 110369156 | |
| 60066 | 110372852 | 110373875 | |
| 60067 | 110430176 | 110431017 | 185950 |
| 60068 | 110433100 | 110434768 | 185950 |
| 60069 | 110436165 | 110440419 | 185950 |
| 60070 | 110455130 | 110456088 | |
| 60071 | 110468310 | 110469350 | |
| 60072 | 110488859 | 110489989 | |
| 60073 | 110519979 | 110523617 | |
| 60074 | 110525910 | 110528852 | |
| 60075 | 110650946 | 110651638 | |
| 60076 | 110658627 | 110659318 | |
| 60077 | 110669712 | 110670477 | |
| 60078 | 110699490 | 110700427 | |
| 60079 | 110713633 | 110717356 | |
| 60080 | 110746387 | 110748623 | |
| 60081 | 110778586 | 110779222 | |
| 60082 | 110789765 | 110791840 | |
| 60083 | 110849091 | 110849823 | 187498 |
| 60084 | 110999793 | 111000392 | 134871 |
| 60085 | 111048284 | 111050698 | 134871 |
| 60086 | 111063935 | 111066012 | 134871 |
| 60087 | 111158666 | 111160911 | 232814, 134871 |
| 60088 | 111172451 | 111174218 | |
| 60089 | 111176122 | 111180572 | 139832 |
| 60090 | 111193183 | 111194339 | 139832 |
| 60091 | 111208058 | 111208627 | 139832 |
| 60092 | 111211563 | 111214728 | 139832 |
| 60093 | 111227794 | 111228954 | |
| 60094 | 111228968 | 111229573 | |
| 60095 | 111267098 | 111270325 | 213995 |
| 60096 | 111357085 | 111359098 | 134905 |
| 60097 | 111363787 | 111366675 | 153487, 134905 |
| 60098 | 111367435 | 111369003 | 153487 |
| 60099 | 111461990 | 111462866 | 259831 |
| 60100 | 111522243 | 111523044 | |
| 60101 | 111564655 | 111569115 | 088448 |
| 60102 | 111617526 | 111618110 | |
| 60103 | 111719555 | 111720633 | |
| 60104 | 111805071 | 111807318 | 102606 |
| 60105 | 112154416 | 112155400 | |
| 60106 | 112186157 | 112187074 | |
| 60107 | 112194766 | 112196024 | |
| 60108 | 112220375 | 112221223 | |
| 60109 | 112266831 | 112267433 | 215881, 204398 |
| 60110 | 112324321 | 112325127 | 204398 |
| 60111 | 112330483 | 112331371 | |
| 60112 | 112867336 | 112868735 | |
| 60113 | 112884185 | 112885364 | |
| 60114 | 112902333 | 112903278 | |
| 60115 | 113127862 | 113128648 | |
| 60116 | 113130948 | 113131897 | |
| 60117 | 113241547 | 113243518 | 126216 |
| 60118 | 113260094 | 113261716 | |
| 60119 | 113298077 | 113298746 | |
| 60120 | 113342950 | 113345707 | 068650 |
| 60121 | 113352022 | 113353056 | 068650 |
| 60122 | 113365687 | 113367319 | 068650 |
| 60123 | 113376687 | 113377582 | 068650 |
| 60124 | 113547631 | 113548829 | 126217 |
| 60125 | 113581257 | 113581929 | 126217 |
| 60126 | 113583043 | 113583861 | 126217 |
| 60127 | 113622502 | 113624112 | 235280, 126217 |
| 60128 | 113638834 | 113640200 | 126217 |
| 60129 | 113759485 | 113760913 | 057593 |
| 60130 | 113776493 | 113778083 | 126218 |
| 60131 | 113791822 | 113792708 | 126218 |
| 60132 | 113803983 | 113805431 | |
| 60133 | 113805994 | 113808478 | |
| 60134 | 113812181 | 113814212 | 126231 |
| 60135 | 113841413 | 113843642 | 126226 |
| 60136 | 113861927 | 113864924 | 139842, 126226 |
| 60137 | 113873390 | 113874973 | 139842 |
| 60138 | 113950624 | 113953137 | 185896 |
| 60139 | 113953953 | 113954791 | 185896 |
| 60140 | 114017329 | 114018648 | 139835 |
| 60141 | 114047782 | 114048781 | |
| 60142 | 114144293 | 114146420 | 150403, 150401 |
| 60143 | 114147014 | 114149149 | 150403 |
| 60144 | 114239068 | 114239881 | 198176 |
| 60145 | 114301504 | 114302271 | |
| 60146 | 114534862 | 114536675 | 233695, 183087 |
| 60147 | 114539100 | 114541508 | 233695, 183087 |
| 60148 | 114541703 | 114544123 | 233695, 183087 |
| 60149 | 114547715 | 114549347 | 183087 |
| 60150 | 114565550 | 114567667 | 272695, 183087 |
| 60151 | 114739688 | 114741430 | |
| 60152 | 114746223 | 114746753 | |
| 60153 | 114829449 | 114831738 | 185989 |
| 60154 | 114880362 | 114881832 | 185989 |
| 60155 | 114884100 | 114885127 | 185989 |
| 60156 | 114917067 | 114917714 | |
| 60157 | 114993383 | 114994759 | |
| 60158 | 115000013 | 115001466 | 130177 |
| 60159 | 115045618 | 115048411 | 169062 |
| 60160 | 115078971 | 115080844 | 198824 |

The locations of ODSCs on Chromosome 14 are shown in Table 56.

TABLE 56
ODSC Locations on Chromosome 14

| SEQ ID NO. | Start | End | Overlapping Gene(s) |
|---|---|---|---|
| 60161 | 20773699 | 20774374 | 258459, 136319 |
| 60162 | 20794997 | 20796059 | 100814 |
| 60163 | 20799180 | 20799839 | 100814 |
| 60164 | 20800355 | 20802464 | 100814 |
| 60165 | 20810085 | 20812806 | 259001, 254846, 129484 |
| 60166 | 20868232 | 20870407 | 129566 |
| 60167 | 20876808 | 20877855 | 129566 |
| 60168 | 20879581 | 20882904 | 129566 |
| 60169 | 20922063 | 20924709 | 100823, 092094 |
| 60170 | 20928865 | 20930439 | 165782 |
| 60171 | 20936778 | 20939295 | 198805 |
| 60172 | 20940592 | 20941281 | 198805 |
| 60173 | 21077176 | 21078957 | 259060, 173464 |
| 60174 | 21081110 | 21082337 | |
| 60175 | 21100917 | 21101736 | |
| 60176 | 21130847 | 21131752 | |
| 60177 | 21148997 | 21157418 | 259171, 214274, 258818 |
| 60178 | 21158906 | 21159788 | 259171, 214274, 258818 |
| 60179 | 21315907 | 21318003 | |
| 60180 | 21438975 | 21440041 | |
| 60181 | 21457155 | 21458953 | 165792 |
| 60182 | 21482783 | 21483779 | |
| 60183 | 21491934 | 21494118 | 258604, 179636, 165795 |
| 60184 | 21506622 | 21508823 | 165795 |
| 60185 | 21509178 | 21512916 | 165799, 165795 |
| 60186 | 21515433 | 21516020 | 165795 |
| 60187 | 21522070 | 21523208 | 165795 |
| 60188 | 21528581 | 21532438 | 165795 |
| 60189 | 21537593 | 21540523 | 165801, 165795 |
| 60190 | 21552966 | 21554141 | 165801 |
| 60191 | 21565120 | 21567334 | 232070, 165804 |
| 60192 | 21571407 | 21573669 | 232070, 165804 |
| 60193 | 21731679 | 21732991 | 092199 |
| 60194 | 21735735 | 21738762 | 092199 |
| 60195 | 21776190 | 21777991 | 092200 |
| 60196 | 21850223 | 21850699 | 092201 |
| 60197 | 21851090 | 21852853 | 260830, 092201 |
| 60198 | 21904615 | 21906387 | 100888 |
| 60199 | 21923080 | 21925290 | 100888 |
| 60200 | 21944583 | 21946299 | 092203, 129472 |
| 60201 | 21978405 | 21980441 | 165819 |
| 60202 | 21994087 | 21994856 | 165821 |
| 60203 | 22025376 | 22026535 | |
| 60204 | 22383067 | 22386581 | 211791 |
| 60205 | 22641133 | 22643142 | |
| 60206 | 22801682 | 22803752 | 256221 |
| 60207 | 22838233 | 22839960 | |
| 60208 | 22859119 | 22859961 | 251002 |
| 60209 | 22896709 | 22897251 | 251002 |
| 60210 | 23039154 | 23039911 | 129562 |
| 60211 | 23041460 | 23041984 | 129562 |
| 60212 | 23056931 | 23059076 | 129562 |
| 60213 | 23066809 | 23069006 | 100439, 228313 |
| 60214 | 23082686 | 23083840 | |
| 60215 | 23122172 | 23122755 | |
| 60216 | 23137275 | 23138963 | |
| 60217 | 23235008 | 23236890 | 155463, 258458 |
| 60218 | 23290932 | 23292853 | 215306, 155465 |
| 60219 | 23293857 | 23294590 | 155465 |
| 60220 | 23298308 | 23300027 | 172590, 155465 |
| 60221 | 23305573 | 23307317 | 157227 |
| 60222 | 23321913 | 23323101 | 212335 |
| 60223 | 23339702 | 23342799 | 197324 |
| 60224 | 23372246 | 23373373 | 100461 |
| 60225 | 23386439 | 23388860 | 237054, 100461 |
| 60226 | 23397607 | 23400266 | 257285, 100462 |
| 60227 | 23424912 | 23426636 | 265037, 092036, 259132 |
| 60228 | 23435718 | 23436218 | 259132 |
| 60229 | 23443227 | 23444403 | 259132, 129474 |
| 60230 | 23445153 | 23452913 | 258457, 259132, 129474 |
| 60231 | 23475280 | 23477292 | 100802 |
| 60232 | 23478682 | 23480143 | 100802 |
| 60233 | 23503144 | 23505319 | 100804 |
| 60234 | 23526614 | 23527705 | 139880 |
| 60235 | 23531025 | 23532269 | 100813 |
| 60236 | 23539559 | 23541138 | 100813 |
| 60237 | 23547015 | 23548833 | 100813 |
| 60238 | 23563585 | 23565713 | 179933, 100813 |
| 60239 | 23714376 | 23714961 | 215277 |
| 60240 | 23754361 | 23756360 | 215271 |
| 60241 | 23769678 | 23771085 | 129473, 235194 |
| 60242 | 23775212 | 23776925 | 258643, 129473 |
| 60243 | 23789419 | 23792103 | 100836, 268702, 258643 |
| 60244 | 23937867 | 23939748 | 129460 |
| 60245 | 24019784 | 24021538 | 136367, 157306 |
| 60246 | 24024732 | 24026644 | 259431, 136367, 157306 |
| 60247 | 24070791 | 24072480 | |
| 60248 | 24077533 | 24079826 | |
| 60249 | 24082566 | 24083155 | |
| 60250 | 24405232 | 24406224 | |
| 60251 | 24422520 | 24423763 | 157326, 215256 |
| 60252 | 24505172 | 24506356 | 225766 |
| 60253 | 24520839 | 24521449 | 186648 |
| 60254 | 24530348 | 24531278 | 186648 |
| 60255 | 24533262 | 24533830 | 186648 |
| 60256 | 24543621 | 24545401 | 100884 |
| 60257 | 24550102 | 24551283 | 129535 |
| 60258 | 24561950 | 24565516 | 100889, 129535 |
| 60259 | 24577844 | 24578514 | 100889, 129535 |
| 60260 | 24582378 | 24585225 | 100897, 129535 |
| 60261 | 24604550 | 24606371 | 092010 |
| 60262 | 24610076 | 24611262 | 100908 |
| 60263 | 24615004 | 24616409 | 092098, 100911 |
| 60264 | 24616440 | 24617625 | 100911, 092098 |
| 60265 | 24629908 | 24631696 | 213928, 259529 |
| 60266 | 24648902 | 24649902 | 196497, 259522, 100918 |
| 60267 | 24657209 | 24658438 | 254692, 100926, 196497, |
| | | | 259522 |
| 60268 | 24663905 | 24666286 | 254692, 100926 |
| 60269 | 24681812 | 24683454 | 213920, 255526, 254692, |
| | | | 254505, 100926, 260669 |
| 60270 | 24684538 | 24685542 | 213920, 260669, 255526 |
| 60271 | 24700460 | 24702873 | 100938, 129559, 255526 |
| 60272 | 24710954 | 24713232 | 092330 |
| 60273 | 24740005 | 24741592 | 100949 |
| 60274 | 24768036 | 24769904 | 196943, 157379 |
| 60275 | 24776099 | 24778151 | 196943, 213906, 136305 |
| 60276 | 24779815 | 24780574 | 213906, 136305, 272658 |
| 60277 | 24801162 | 24802259 | 258973, 129467 |
| 60278 | 24802711 | 24803580 | 258973, 129467 |
| 60279 | 24812919 | 24813868 | |
| 60280 | 24822359 | 24823987 | |
| 60281 | 24855881 | 24856415 | |
| 60282 | 24872532 | 24872968 | 205978 |
| 60283 | 24887429 | 24888284 | 205978 |
| 60284 | 24894621 | 24895278 | |
| 60285 | 24895811 | 24896639 | 139899 |
| 60286 | 24898275 | 24900763 | 100441, 139899 |
| 60287 | 24910874 | 24913042 | 100445 |
| 60288 | 25278333 | 25278693 | |
| 60289 | 25279210 | 25280236 | 168952 |
| 60290 | 25284678 | 25288355 | 168952 |
| 60291 | 25307731 | 25310104 | 168952 |
| 60292 | 25330294 | 25331011 | 168952 |
| 60293 | 25666886 | 25667478 | |
| 60294 | 26750586 | 26751350 | |
| 60295 | 27233989 | 27236139 | 257842 |
| 60296 | 27244286 | 27246510 | 257845, 257842 |
| 60297 | 27277444 | 27278086 | 257845 |
| 60298 | 27309649 | 27312713 | 258081 |
| 60299 | 29226324 | 29226767 | 257126 |
| 60300 | 29497958 | 29498977 | 258028 |
| 60301 | 30054134 | 30054586 | 184304 |
| 60302 | 30315787 | 30316500 | 184304 |
| 60303 | 30692445 | 30693649 | 248975 |
| 60304 | 30858260 | 30859310 | |
| 60305 | 31027601 | 31028887 | 092140, 257636 |
| 60306 | 31090428 | 31092453 | 092108 |
| 60307 | 31317699 | 31318472 | |
| 60308 | 31463857 | 31464583 | 196792 |
| 60309 | 31494134 | 31496771 | 100478, 196792 |
| 60310 | 31500212 | 31503808 | 100478 |
| 60311 | 31528630 | 31529335 | 100478 |
| 60312 | 31559073 | 31561536 | 100478 |
| 60313 | 31634239 | 31635408 | 092148 |
| 60314 | 31664194 | 31664761 | 092148 |
| 60315 | 31675009 | 31678350 | 092148 |
| 60316 | 31697430 | 31700168 | |
| 60317 | 31726970 | 31727928 | 199291, 257831 |
| 60318 | 31730667 | 31732472 | 257831 |
| 60319 | 31732714 | 31733803 | 257831 |
| 60320 | 31734916 | 31735530 | 257831 |
| 60321 | 31830157 | 31831143 | 129493, 203546 |
| 60322 | 31888657 | 31890467 | 250365, 129493, 203546 |
| 60323 | 31925774 | 31928013 | 129480, 203546 |
| 60324 | 32030252 | 32031643 | 257155, 151413 |
| 60325 | 32324353 | 32324986 | 151413 |
| 60326 | 32349590 | 32351612 | |
| 60327 | 32403780 | 32408031 | |
| 60328 | 32413063 | 32414667 | 258474 |
| 60329 | 32434672 | 32437717 | |
| 60330 | 32462023 | 32463400 | |
| 60331 | 32544598 | 32548662 | 258655, 100852 |
| 60332 | 32669927 | 32673674 | 201654, 202337 |
| 60333 | 32689957 | 32690553 | |
| 60334 | 32698439 | 32700891 | |
| 60335 | 32725571 | 32727220 | |
| 60336 | 32728653 | 32729374 | |
| 60337 | 32772981 | 32773623 | |
| 60338 | 32776831 | 32778011 | |
| 60339 | 32848544 | 32849026 | 151320, 258580 |
| 60340 | 32937150 | 32939081 | 151320 |
| 60341 | 32941011 | 32943184 | 151320 |
| 60342 | 32976252 | 32977831 | 151320 |
| 60343 | 32978430 | 32979608 | 151320 |
| 60344 | 32982677 | 32984500 | 151320 |
| 60345 | 33027643 | 33030081 | 151320 |
| 60346 | 33104143 | 33104891 | 151320 |
| 60347 | 33365889 | 33367265 | |
| 60348 | 33407293 | 33409237 | 151322 |
| 60349 | 34407678 | 34408175 | 129521 |
| 60350 | 34458288 | 34459754 | 129521 |
| 60351 | 34504488 | 34505869 | 129521 |
| 60352 | 34512626 | 34513443 | 258897, 129521 |
| 60353 | 34529036 | 34530243 | 129521 |
| 60354 | 34550326 | 34551803 | 129521 |
| 60355 | 34719354 | 34720659 | 129521 |
| 60356 | 34726598 | 34728100 | 129521 |
| 60357 | 34766413 | 34767836 | 129521 |
| 60358 | 34893322 | 34896455 | 129521 |
| 60359 | 34930031 | 34932353 | 165389, 129521 |
| 60360 | 35007580 | 35009289 | 129518 |
| 60361 | 35098376 | 35099532 | 129515 |
| 60362 | 35156527 | 35157261 | |
| 60363 | 35168597 | 35169834 | |
| 60364 | 35182132 | 35184778 | 165410 |
| 60365 | 35187063 | 35189001 | |
| 60366 | 35312053 | 35313006 | 198604 |
| 60367 | 35342688 | 35345260 | 258738, 198604 |
| 60368 | 35449822 | 35453146 | 100883, 258704 |
| 60369 | 35514501 | 35516853 | 151327 |
| 60370 | 35519324 | 35520462 | 151327 |
| 60371 | 35547102 | 35547695 | 151327 |
| 60372 | 35586278 | 35586840 | 092020 |
| 60373 | 35590131 | 35592522 | 100890, 258790, 092020 |
| 60374 | 35625073 | 35625992 | 100890, 258790 |
| 60375 | 35628455 | 35631262 | 100890, 258790 |
| 60376 | 35682190 | 35685103 | 100890, 258790 |
| 60377 | 35756597 | 35757424 | 100902, 258790 |
| 60378 | 35760988 | 35762604 | 100902, 258790 |
| 60379 | 35804431 | 35807634 | |
| 60380 | 35810078 | 35812065 | |
| 60381 | 35821594 | 35823560 | |
| 60382 | 35824835 | 35826277 | |
| 60383 | 35833860 | 35836715 | |
| 60384 | 35843304 | 35843997 | |
| 60385 | 35866329 | 35868038 | |
| 60386 | 35868054 | 35875546 | 100906 |
| 60387 | 36277853 | 36278783 | 266264, 174373 |
| 60388 | 36295129 | 36296552 | 100916, 258938 |
| 60389 | 36345079 | 36349262 | 100916 |
| 60390 | 36523399 | 36524365 | 257720, 258342 |
| 60391 | 36526924 | 36527324 | 258342 |
| 60392 | 36537845 | 36539348 | |
| 60393 | 36560149 | 36560890 | 257585 |
| 60394 | 36769270 | 36770160 | 151332 |
| 60395 | 36786796 | 36787748 | 151332 |
| 60396 | 36788457 | 36790380 | 151332 |
| 60397 | 36899200 | 36900625 | |
| 60398 | 36923740 | 36924390 | |
| 60399 | 37585991 | 37586952 | 183032 |
| 60400 | 37640978 | 37642295 | 258708, 183032 |
| 60401 | 37666259 | 37667859 | 151338 |
| 60402 | 37758747 | 37759444 | 151338 |
| 60403 | 37798389 | 37799015 | 151338 |
| 60404 | 37935155 | 37936352 | 151338 |
| 60405 | 37949783 | 37950917 | 151338 |
| 60406 | 38015185 | 38016747 | 151338 |
| 60407 | 38035457 | 38036834 | 259087, 258414 |
| 60408 | 38048768 | 38050564 | |
| 60409 | 38051968 | 38054001 | |
| 60410 | 38054922 | 38066016 | 129514, 139865 |
| 60411 | 38068337 | 38069608 | 129514, 139865 |
| 60412 | 38071669 | 38072767 | 139865 |
| 60413 | 38166164 | 38167293 | 139865 |
| 60414 | 38260345 | 38261258 | 139865 |
| 60415 | 38334075 | 38334901 | 139865 |
| 60416 | 38343692 | 38344613 | 139865 |
| 60417 | 38676693 | 38679009 | 139874 |
| 60418 | 38759722 | 38760328 | 259072 |
| 60419 | 38860253 | 38861270 | |
| 60420 | 39337623 | 39338440 | 259070 |
| 60421 | 39492511 | 39493855 | |
| 60422 | 39519537 | 39520075 | 100934 |
| 60423 | 39570937 | 39573436 | 258651, 100934 |
| 60424 | 39638179 | 39640391 | 182400 |
| 60425 | 39643281 | 39645882 | 259083, 100941 |
| 60426 | 39702750 | 39703960 | 258941, 150526 |
| 60427 | 39734247 | 39737391 | 258940, 150527, 258941 |
| 60428 | 39845782 | 39846664 | 150527 |
| 60429 | 39900179 | 39902785 | 165355 |
| 60430 | 41704459 | 41705240 | |
| 60431 | 43641975 | 43642941 | |
| 60432 | 44247726 | 44248951 | |
| 60433 | 44450464 | 44451241 | |
| 60434 | 44478814 | 44480278 | 258969 |
| 60435 | 44897299 | 44898261 | |
| 60436 | 45365808 | 45367545 | 179476, 258633 |
| 60437 | 45430147 | 45432164 | 198718, 179454 |
| 60438 | 45552733 | 45554258 | 185246, 249163 |
| 60439 | 45602759 | 45605529 | 187790, 100442 |
| 60440 | 45721236 | 45723892 | 129534 |
| 60441 | 46956796 | 46958156 | 258700 |
| 60442 | 47959436 | 47960912 | 272781, 139915 |
| 60443 | 48802283 | 48803563 | |
| 60444 | 49432208 | 49433092 | |
| 60445 | 49946052 | 49946602 | |
| 60446 | 50016539 | 50017846 | |
| 60447 | 50051856 | 50054034 | 258486, 213741 |
| 60448 | 50064292 | 50066598 | 165501, 253459, 213741 |
| 60449 | 50086223 | 50091553 | 258377, 168282, 165502 |
| 60450 | 50099678 | 50102546 | 165506 |
| 60451 | 50107452 | 50109142 | |
| 60452 | 50154424 | 50155395 | 100479 |
| 60453 | 50159476 | 50160692 | 197776 |
| 60454 | 50233556 | 50237783 | 165516 |
| 60455 | 50318512 | 50320822 | 266037, 165525 |
| 60456 | 50327792 | 50331185 | 265150 |
| 60457 | 50334114 | 50335824 | |
| 60458 | 50358441 | 50361179 | 165527 |
| 60459 | 50431705 | 50434469 | |
| 60460 | 50438613 | 50440121 | |
| 60461 | 50452569 | 50455965 | 214900 |
| 60462 | 50458311 | 50460816 | 214900 |
| 60463 | 50465819 | 50471293 | 214900 |
| 60464 | 50484634 | 50486681 | 258929 |
| 60465 | 50504427 | 50507962 | 273065, 258929 |
| 60466 | 50510278 | 50513227 | 273307, 273065 |
| 60467 | 50526729 | 50529991 | 273307, 273065 |
| 60468 | 50582580 | 50584115 | 100485, 100483 |
| 60469 | 50691755 | 50693029 | 100485 |
| 60470 | 50696953 | 50699429 | 100485 |
| 60471 | 50770455 | 50770942 | 087299 |
| 60472 | 50778080 | 50780036 | 125375, 087299 |
| 60473 | 50808953 | 50809997 | 100490 |
| 60474 | 50856416 | 50857006 | 100490 |
| 60475 | 50862861 | 50864111 | 258857, 100490 |
| 60476 | 50908234 | 50909136 | 012983 |
| 60477 | 50913281 | 50914638 | 012983 |
| 60478 | 50996379 | 51000192 | 198513, 012983 |
| 60479 | 51133540 | 51135956 | 151748 |
| 60480 | 51283146 | 51283599 | 100503 |
| 60481 | 51296695 | 51298479 | 100503 |
| 60482 | 51386117 | 51386600 | 100504 |
| 60483 | 51401992 | 51402964 | 100504 |
| 60484 | 51403912 | 51404538 | 100504 |
| 60485 | 51409455 | 51412436 | 100504 |
| 60486 | 51421688 | 51422901 | |
| 60487 | 51470412 | 51474656 | 100505 |
| 60488 | 51495279 | 51497079 | 100505 |
| 60489 | 51543960 | 51545115 | 100505 |
| 60490 | 51561815 | 51563101 | 100505 |
| 60491 | 51613915 | 51614388 | |
| 60492 | 51706712 | 51707682 | 139921 |
| 60493 | 51759715 | 51760653 | |
| 60494 | 51808087 | 51809976 | 258479 |
| 60495 | 51897127 | 51900204 | |
| 60496 | 51908632 | 51910546 | |
| 60497 | 51914473 | 51916887 | |
| 60498 | 51999216 | 52002617 | 139926, 258537 |
| 60499 | 52005022 | 52008361 | 139926, 258537 |
| 60500 | 52011933 | 52015689 | 139926, 258537 |
| 60501 | 52018783 | 52023164 | 139926, 258537 |
| 60502 | 52083541 | 52084293 | 139926 |
| 60503 | 52104594 | 52105779 | 139926 |
| 60504 | 52117084 | 52120488 | 139926 |
| 60505 | 52455727 | 52457054 | 087302 |
| 60506 | 52474308 | 52474909 | 087303 |
| 60507 | 52924497 | 52926020 | 087301 |
| 60508 | 53018822 | 53020957 | 180998, 087301 |
| 60509 | 53160689 | 53163472 | 197930 |
| 60510 | 53166996 | 53168557 | |
| 60511 | 53172921 | 53174940 | 100519 |
| 60512 | 53196587 | 53197880 | 198252 |
| 60513 | 53256837 | 53259440 | 100522 |
| 60514 | 53288241 | 53289088 | |
| 60515 | 53372426 | 53374232 | 073712 |
| 60516 | 53392848 | 53393389 | 073712 |
| 60517 | 53414532 | 53415275 | 073712 |
| 60518 | 53416220 | 53418769 | 073712 |
| 60519 | 53465234 | 53465890 | |
| 60520 | 53498733 | 53499600 | |
| 60521 | 53509105 | 53509734 | |
| 60522 | 53618587 | 53620717 | 258731, 266552, 100523 |
| 60523 | 54080125 | 54081456 | 225680, 237356 |
| 60524 | 54181936 | 54182505 | |
| 60525 | 54309313 | 54310638 | 235269 |
| 60526 | 54317216 | 54319138 | 235269 |
| 60527 | 54321497 | 54322655 | |
| 60528 | 54347870 | 54348606 | |
| 60529 | 54357228 | 54357951 | |
| 60530 | 54413307 | 54413943 | |
| 60531 | 54419246 | 54421067 | 125378 |
| 60532 | 54446706 | 54447267 | |
| 60533 | 54600450 | 54601103 | |
| 60534 | 54604748 | 54605901 | |
| 60535 | 54663941 | 54664646 | |
| 60536 | 54820764 | 54821249 | |
| 60537 | 54831073 | 54832359 | |
| 60538 | 54863377 | 54863910 | 100526 |
| 60539 | 54906378 | 54908798 | 100528 |
| 60540 | 54913232 | 54914631 | |
| 60541 | 54954761 | 54956540 | 197045 |
| 60542 | 54975998 | 54977384 | 100532 |
| 60543 | 55030604 | 55035484 | 020577 |
| 60544 | 55036549 | 55038512 | 020577 |
| 60545 | 55044598 | 55045169 | 020577 |
| 60546 | 55069982 | 55070506 | 020577 |
| 60547 | 55116674 | 55121548 | 020577 |
| 60548 | 55150909 | 55151348 | 020577 |
| 60549 | 55152315 | 55154664 | 020577 |
| 60550 | 55185559 | 55186870 | 020577 |
| 60551 | 55196854 | 55197763 | 020577 |
| 60552 | 55239366 | 55240494 | 020577 |
| 60553 | 55250140 | 55252429 | 020577 |
| 60554 | 55301448 | 55302527 | |
| 60555 | 55304156 | 55307852 | |
| 60556 | 55311126 | 55311652 | 131979 |
| 60557 | 55347927 | 55349115 | 131979 |
| 60558 | 55367042 | 55370271 | 131979 |
| 60559 | 55493131 | 55494732 | 180008, 198554 |
| 60560 | 55517488 | 55519810 | 168175 |
| 60561 | 55569776 | 55571138 | |
| 60562 | 55596062 | 55597286 | 131981 |
| 60563 | 55737608 | 55739239 | 178974, 258413 |
| 60564 | 55768284 | 55768875 | 178974 |
| 60565 | 55847614 | 55848825 | 126775 |
| 60566 | 55877480 | 55879459 | 126775 |
| 60567 | 56045856 | 56048345 | 186615, 126777 |
| 60568 | 56065750 | 56067879 | 126777 |
| 60569 | 56072331 | 56073127 | 126777 |
| 60570 | 56269648 | 56270799 | |
| 60571 | 56273138 | 56274710 | |
| 60572 | 56298179 | 56299502 | |
| 60573 | 56584607 | 56586509 | 139946 |
| 60574 | 56806610 | 56807280 | 259719 |
| 60575 | 57045959 | 57047652 | 070269 |
| 60576 | 57050431 | 57051149 | 070269 |
| 60577 | 57116583 | 57117463 | 070269, 259133 |
| 60578 | 57524114 | 57525094 | 248550 |
| 60579 | 57526846 | 57527684 | 248550 |
| 60580 | 57734565 | 57736775 | 053770, 070367 |
| 60581 | 57856331 | 57858867 | 139977 |
| 60582 | 57868391 | 57869940 | 139977 |
| 60583 | 57953634 | 57955401 | 100557 |
| 60584 | 57958088 | 57961818 | 100557 |
| 60585 | 57967835 | 57968226 | |
| 60586 | 58548688 | 58550008 | 139971 |
| 60587 | 58637144 | 58638122 | 139971 |
| 60588 | 58666376 | 58667793 | 131966, 139971 |
| 60589 | 58705335 | 58707312 | 139971 |
| 60590 | 58710233 | 58713025 | 100567, 139971 |
| 60591 | 58738936 | 58739907 | 257621, 139971 |
| 60592 | 58747291 | 58750896 | 257621, 139971 |
| 60593 | 58754006 | 58755697 | 268466, 257621, 139971 |
| 60594 | 58762937 | 58766789 | 032219, 257621, 139971 |
| 60595 | 58861936 | 58863321 | 196860, 258658 |
| 60596 | 58893012 | 58895590 | 100578, 258378, 100575 |
| 60597 | 59130320 | 59131068 | |
| 60598 | 59212689 | 59213455 | |
| 60599 | 59219482 | 59220121 | |
| 60600 | 59275441 | 59275928 | |
| 60601 | 59296200 | 59296908 | 258583 |
| 60602 | 59603894 | 59604492 | |
| 60603 | 59654564 | 59656596 | 100592 |
| 60604 | 59682011 | 59682759 | 100592 |
| 60605 | 59741548 | 59746247 | 100592 |
| 60606 | 59747657 | 59751243 | 100592 |
| 60607 | 59763316 | 59764486 | 100592 |
| 60608 | 59781696 | 59783136 | 100592 |
| 60609 | 59800396 | 59800881 | 100592 |
| 60610 | 59824248 | 59825337 | 100592 |
| 60611 | 59827542 | 59828290 | 100592 |
| 60612 | 59873249 | 59874333 | |
| 60613 | 59947875 | 59952198 | 050130, 126790, 258782 |
| 60614 | 60174471 | 60175607 | 139970 |
| 60615 | 60254578 | 60255637 | 139970 |
| 60616 | 60315094 | 60316688 | 139970 |
| 60617 | 60386112 | 60387509 | 261120, 131951 |
| 60618 | 60470500 | 60471124 | 131951, 258553 |
| 60619 | 60557673 | 60560075 | 126773, 258553 |
| 60620 | 60628382 | 60628949 | 100612, 126773 |
| 60621 | 60630379 | 60632651 | 100612, 126773 |
| 60622 | 60645818 | 60646547 | 258563 |
| 60623 | 60714384 | 60717412 | 254718, 100614 |
| 60624 | 61091773 | 61092887 | |
| 60625 | 61123371 | 61124741 | 126778 |
| 60626 | 61157977 | 61158469 | |
| 60627 | 61189152 | 61189515 | 100625 |
| 60628 | 61201169 | 61202221 | 020426 |
| 60629 | 61286185 | 61287020 | 020426 |
| 60630 | 61446932 | 61448932 | 139974, 126814, 258892 |
| 60631 | 61568868 | 61571772 | |
| 60632 | 61644335 | 61646975 | |
| 60633 | 61655684 | 61656303 | 027075 |
| 60634 | 61727305 | 61728333 | 027075 |
| 60635 | 61744422 | 61745806 | 027075, 182107 |
| 60636 | 61746891 | 61748440 | 027075, 182107 |
| 60637 | 61754810 | 61755503 | 027075 |
| 60638 | 61788087 | 61788819 | 027075, 247287 |
| 60639 | 61833901 | 61834389 | 027075 |
| 60640 | 61905247 | 61906438 | 027075 |
| 60641 | 61927125 | 61929047 | 027075 |
| 60642 | 61931965 | 61934593 | 027075 |
| 60643 | 61937309 | 61940552 | 027075 |
| 60644 | 61941841 | 61945330 | 027075 |
| 60645 | 61946145 | 61947368 | 027075 |
| 60646 | 61955891 | 61959085 | 027075 |
| 60647 | 61984080 | 61985398 | 027075 |
| 60648 | 61990404 | 61994647 | 027075 |
| 60649 | 62002484 | 62006044 | 258926, 258989, 027075 |
| 60650 | 62009729 | 62010742 | 258989, 027075, 258926 |
| 60651 | 62018050 | 62023124 | 250548, 258989 |
| 60652 | 62026406 | 62032236 | 258989, 250548 |
| 60653 | 62040141 | 62041888 | 258989, 232774 |
| 60654 | 62161243 | 62165843 | 100644, 258777 |
| 60655 | 62219724 | 62223157 | 258964 |
| 60656 | 63158171 | 63160190 | |
| 60657 | 63162885 | 63163933 | |
| 60658 | 63902398 | 63903301 | 154001 |
| 60659 | 63904609 | 63905362 | 154001 |
| 60660 | 63934850 | 63935462 | 154001 |
| 60661 | 63937654 | 63938964 | 154001 |
| 60662 | 64008780 | 64011223 | 261242, 154001 |
| 60663 | 64107585 | 64109448 | 140006 |
| 60664 | 64193094 | 64195585 | 126821 |
| 60665 | 64318986 | 64320970 | 054654 |
| 60666 | 64330886 | 64331567 | 054654 |
| 60667 | 64360609 | 64361135 | 054654 |
| 60668 | 64389689 | 64390308 | 054654 |
| 60669 | 64536202 | 64537574 | 054654 |
| 60670 | 64576210 | 64577157 | 054654, 140009 |
| 60671 | 64579448 | 64581299 | 054654, 140009 |
| 60672 | 64583670 | 64586205 | 054654, 140009 |
| 60673 | 64589571 | 64590713 | 054654, 140009 |
| 60674 | 64804693 | 64806164 | 140009, 214770 |
| 60675 | 64853894 | 64856334 | 100714 |
| 60676 | 64867403 | 64868118 | 100714 |
| 60677 | 64868534 | 64869176 | 100714 |
| 60678 | 64871713 | 64872443 | 100714 |
| 60679 | 64876798 | 64877726 | 100714 |
| 60680 | 64904826 | 64905428 | 100714, 258824 |
| 60681 | 64967628 | 64968957 | 089775 |
| 60682 | 64969236 | 64972749 | 126804, 089775 |
| 60683 | 65005833 | 65008132 | 259116, 126803 |
| 60684 | 65078179 | 65079144 | |
| 60685 | 65106154 | 65106712 | |
| 60686 | 65114958 | 65117095 | |
| 60687 | 65120536 | 65122200 | |
| 60688 | 65123493 | 65124236 | |
| 60689 | 65170130 | 65176387 | 126822 |
| 60690 | 65177360 | 65178195 | 126822 |
| 60691 | 65184114 | 65189082 | 126822 |
| 60692 | 65190443 | 65194270 | 126822 |
| 60693 | 65380917 | 65382179 | 125954, 257365, 258289 |
| 60694 | 65404090 | 65406315 | 176153, 125954, 257365, |
| | | | 258289 |
| 60695 | 65408724 | 65409904 | 176153, 125954, 257365, |
| | | | 258289 |
| 60696 | 65414787 | 65415631 | 125954, 257365, 139998 |
| 60697 | 65437995 | 65439750 | 139998, 125954, 257365 |
| 60698 | 65453293 | 65454799 | 125954, 257365 |
| 60699 | 65501635 | 65502307 | 125952, 125954, 257365 |
| 60700 | 65568079 | 65570562 | 125952 |
| 60701 | 65646531 | 65647771 | |
| 60702 | 65878137 | 65880319 | 033170 |
| 60703 | 66845501 | 66846815 | 258561 |
| 60704 | 66949802 | 66950669 | 258561 |
| 60705 | 66962048 | 66964167 | 258561, 196553 |
| 60706 | 66967378 | 66968796 | 258561 |
| 60707 | 66973534 | 66976451 | 171723, 258561 |
| 60708 | 67080002 | 67080623 | 171723 |
| 60709 | 67224579 | 67225327 | 171723 |
| 60710 | 67238818 | 67239274 | 171723 |
| 60711 | 67397797 | 67400727 | 171723 |
| 60712 | 67706571 | 67709961 | 072415 |
| 60713 | 67729848 | 67730685 | 072415 |
| 60714 | 67825385 | 67828347 | 134001, 100554 |
| 60715 | 67878131 | 67879500 | 100558 |
| 60716 | 67895138 | 67895963 | |
| 60717 | 67999170 | 68000600 | 054690, 198133 |
| 60718 | 68066167 | 68067611 | 100564 |
| 60719 | 68086084 | 68086777 | 081181 |
| 60720 | 68140207 | 68142446 | 100568, 258466 |
| 60721 | 68161449 | 68162708 | 072042 |
| 60722 | 68195495 | 68196450 | 139988, 072121 |
| 60723 | 68204884 | 68206512 | 072121 |
| 60724 | 68282387 | 68284195 | 072121 |
| 60725 | 68400675 | 68401294 | 182185 |
| 60726 | 68407053 | 68412254 | 182185 |
| 60727 | 68571031 | 68571762 | 182185 |
| 60728 | 68603839 | 68609130 | 182185 |
| 60729 | 68630621 | 68633021 | 182185 |
| 60730 | 68636691 | 68641660 | 182185 |
| 60731 | 68647858 | 68649382 | 182185 |
| 60732 | 68655060 | 68655591 | 182185 |
| 60733 | 68656878 | 68662330 | 182185 |
| 60734 | 68664768 | 68667015 | 182185 |
| 60735 | 68676870 | 68678967 | 182185 |
| 60736 | 68695558 | 68701184 | 182185 |
| 60737 | 68712270 | 68718126 | 182185 |
| 60738 | 68773278 | 68775570 | 182185 |
| 60739 | 68924184 | 68951144 | 182185 |
| 60740 | 68951255 | 68955699 | 182185 |
| 60741 | 68984944 | 68991157 | 182185 |
| 60742 | 68992054 | 68995500 | 182185 |
| 60743 | 69010487 | 69016149 | 182185 |
| 60744 | 69016606 | 69017685 | 182185 |
| 60745 | 69026124 | 69033369 | 182185 |
| 60746 | 69036037 | 69038238 | 182185 |
| 60747 | 69042774 | 69045745 | 182185 |
| 60748 | 69086115 | 69088085 | 182185 |
| 60749 | 69091486 | 69098406 | 259038, 182185 |
| 60750 | 69138755 | 69140088 | 182185 |
| 60751 | 69146338 | 69150796 | 258623, 182185 |
| 60752 | 69189923 | 69191134 | 182185 |
| 60753 | 69245306 | 69246177 | |
| 60754 | 69249255 | 69263886 | 185650 |
| 60755 | 69273744 | 69274362 | |
| 60756 | 69280735 | 69284862 | |
| 60757 | 69285813 | 69293152 | |
| 60758 | 69350354 | 69353020 | 072110 |
| 60759 | 69390176 | 69392161 | 072110 |
| 60760 | 69402327 | 69409000 | 258531, 072110 |
| 60761 | 69411984 | 69413875 | 072110 |
| 60762 | 69417308 | 69419680 | 072110 |
| 60763 | 69442283 | 69447320 | 259062, 072110 |
| 60764 | 69468798 | 69469189 | |
| 60765 | 69472429 | 69472951 | |
| 60766 | 69504354 | 69506770 | |
| 60767 | 69509548 | 69510534 | |
| 60768 | 69516259 | 69516936 | |
| 60769 | 69544154 | 69548999 | 139990 |
| 60770 | 69595906 | 69596506 | 139990 |
| 60771 | 69599679 | 69600730 | 139990 |
| 60772 | 69617585 | 69620961 | 139990 |
| 60773 | 69657616 | 69659195 | 081177, 258957 |
| 60774 | 69727797 | 69728842 | 100626 |
| 60775 | 69864096 | 69866309 | 029364, 100632 |
| 60776 | 70003454 | 70004008 | |
| 60777 | 70006401 | 70007162 | |
| 60778 | 70046521 | 70047763 | |
| 60779 | 70072555 | 70074119 | |
| 60780 | 70077343 | 70079959 | 100647 |
| 60781 | 70104065 | 70105160 | 100647 |
| 60782 | 70109463 | 70111352 | 100647 |
| 60783 | 70114568 | 70116236 | 100647 |
| 60784 | 70158977 | 70159898 | 100647 |
| 60785 | 70193217 | 70194271 | 100650 |
| 60786 | 70207294 | 70208317 | 100650 |
| 60787 | 70216647 | 70217406 | 100650 |
| 60788 | 70232920 | 70235270 | 100650 |
| 60789 | 70246617 | 70247304 | 100652 |
| 60790 | 70258887 | 70264991 | 100652 |
| 60791 | 70275696 | 70276467 | |
| 60792 | 70312445 | 70313027 | |
| 60793 | 70316577 | 70318099 | |
| 60794 | 70343204 | 70344859 | 198732 |
| 60795 | 70345620 | 70347597 | 198732 |
| 60796 | 70352724 | 70357049 | 242071, 198732 |
| 60797 | 70403962 | 70404938 | 198732 |
| 60798 | 70408639 | 70409591 | 198732 |
| 60799 | 70471522 | 70472688 | 198732 |
| 60800 | 70499124 | 70499617 | |
| 60801 | 70524171 | 70524785 | 100678 |
| 60802 | 70527660 | 70528435 | 100678 |
| 60803 | 70784066 | 70785823 | |
| 60804 | 70825289 | 70827071 | 133983, 258644 |
| 60805 | 70882530 | 70884241 | 258644, 213463 |
| 60806 | 70915229 | 70916346 | 257759 |
| 60807 | 71066803 | 71068367 | 133997, 259115 |
| 60808 | 71098478 | 71099188 | 259115, 245466 |
| 60809 | 71107582 | 71109580 | 133985, 259115, 245466 |
| 60810 | 71128920 | 71133716 | 133985 |
| 60811 | 71183192 | 71184026 | |
| 60812 | 71219247 | 71223358 | 006432 |
| 60813 | 71246357 | 71246865 | 006432 |
| 60814 | 71275214 | 71277235 | 259153, 006432 |
| 60815 | 71288219 | 71289306 | |
| 60816 | 71293929 | 71295249 | |
| 60817 | 71373166 | 71375772 | 100731 |
| 60818 | 71583026 | 71587156 | |
| 60819 | 71610136 | 71611218 | |
| 60820 | 71786687 | 71789037 | 197555, 259146 |
| 60821 | 71802850 | 71804073 | 197555 |
| 60822 | 71838579 | 71839755 | 197555 |
| 60823 | 72021517 | 72023905 | 197555 |
| 60824 | 72930019 | 72930535 | 182732 |
| 60825 | 73034991 | 73035945 | 258871 |
| 60826 | 73049327 | 73050869 | 258871 |
| 60827 | 73079834 | 73080427 | |
| 60828 | 73131472 | 73133679 | 205683 |
| 60829 | 73267836 | 73268439 | 205683 |
| 60830 | 73278956 | 73280152 | 205683 |
| 60831 | 73295024 | 73295985 | 205683 |
| 60832 | 73316657 | 73317684 | 205683 |
| 60833 | 73359339 | 73362202 | 205683 |
| 60834 | 73392638 | 73394234 | 119599 |
| 60835 | 73492925 | 73494299 | 165861 |
| 60836 | 73524177 | 73526377 | 119707, 258813 |
| 60837 | 73570750 | 73571628 | 119707 |
| 60838 | 73602924 | 73604392 | 080815 |
| 60839 | 73628169 | 73628758 | 080815 |
| 60840 | 73646939 | 73647545 | 080815 |
| 60841 | 73676308 | 73677551 | 080815 |
| 60842 | 73694442 | 73696327 | |
| 60843 | 73736624 | 73737379 | 100767 |
| 60844 | 73763011 | 73763630 | 133961 |
| 60845 | 73894742 | 73895620 | 133961 |
| 60846 | 73923805 | 73926208 | 133961 |
| 60847 | 73928125 | 73930617 | 251393, 133961 |
| 60848 | 73957318 | 73958700 | 255242, 170468, 187105 |
| 60849 | 73992637 | 73993933 | 258695, 187105 |
| 60850 | 74034904 | 74037341 | 119673 |
| 60851 | 74058136 | 74059443 | 177465 |
| 60852 | 74078920 | 74080359 | 205669 |
| 60853 | 74099941 | 74101471 | 258603 |
| 60854 | 74110740 | 74111805 | 119661 |
| 60855 | 74129102 | 74129561 | 119661 |
| 60856 | 74179723 | 74181391 | 176903 |
| 60857 | 74189535 | 74192113 | 156030 |
| 60858 | 74195227 | 74195934 | 156030 |
| 60859 | 74207599 | 74208914 | 156030 |
| 60860 | 74220096 | 74222732 | 156030 |
| 60861 | 74224317 | 74230002 | 264741, 156030 |
| 60862 | 74230992 | 74232189 | 156030 |
| 60863 | 74238466 | 74239350 | 156030 |
| 60864 | 74249748 | 74250458 | 156030 |
| 60865 | 74251664 | 74255363 | 259065, 156030 |
| 60866 | 74261251 | 74263748 | 259065 |
| 60867 | 74291810 | 74292739 | 258586 |
| 60868 | 74303257 | 74303888 | |
| 60869 | 74317494 | 74319911 | 258653, 140043 |
| 60870 | 74352473 | 74354708 | 119725, 140043, 258653 |
| 60871 | 74409731 | 74410178 | 156050 |
| 60872 | 74442744 | 74443348 | 187097 |
| 60873 | 74455318 | 74455722 | 187097 |
| 60874 | 74461648 | 74464684 | 187097 |
| 60875 | 74480841 | 74481484 | 187097 |
| 60876 | 74484583 | 74486768 | 119636, 187097 |
| 60877 | 74529280 | 74529898 | 259114, 119711, 119636 |
| 60878 | 74550373 | 74552268 | 205659, 119711 |
| 60879 | 74600236 | 74601624 | 205659 |
| 60880 | 74606832 | 74607661 | 205659 |
| 60881 | 74608131 | 74609835 | 205659 |
| 60882 | 74684304 | 74684962 | |
| 60883 | 74723881 | 74725055 | 119614 |
| 60884 | 74768791 | 74770212 | 133980, 119688 |
| 60885 | 74905107 | 74905599 | |
| 60886 | 74959344 | 74961137 | 165898, 119655 |
| 60887 | 75078726 | 75079685 | 119681 |
| 60888 | 75084900 | 75085830 | |
| 60889 | 75179080 | 75180576 | 119616, 119682, 214670 |
| 60890 | 75229064 | 75231261 | 119596 |
| 60891 | 75301245 | 75301865 | 119596 |
| 60892 | 75347613 | 75349849 | 119689 |
| 60893 | 75364985 | 75366056 | 119689 |
| 60894 | 75372110 | 75372962 | 198208 |
| 60895 | 75389134 | 75390217 | 198208 |
| 60896 | 75411812 | 75412879 | 119630 |
| 60897 | 75469095 | 75470558 | 119718 |
| 60898 | 75507893 | 75509637 | 119684 |
| 60899 | 75517169 | 75518495 | 119684 |
| 60900 | 75534878 | 75537243 | 119640, 119703 |
| 60901 | 75592533 | 75595131 | 259138, 119638 |
| 60902 | 75625780 | 75626315 | 170348 |
| 60903 | 75642437 | 75643973 | 170348 |
| 60904 | 75683088 | 75683575 | |
| 60905 | 75720860 | 75722024 | 258740 |
| 60906 | 75743395 | 75744356 | |
| 60907 | 75744731 | 75745881 | 170345 |
| 60908 | 75760245 | 75763272 | 259687, 119660 |
| 60909 | 75866366 | 75867816 | |
| 60910 | 75885986 | 75886504 | |
| 60911 | 75893595 | 75896193 | 140044, 259319 |
| 60912 | 75907359 | 75908172 | 140044 |
| 60913 | 75916227 | 75917883 | 140044 |
| 60914 | 75918964 | 75920270 | 140044 |
| 60915 | 75985945 | 75990815 | 156127 |
| 60916 | 75997354 | 75999327 | 156127 |
| 60917 | 75999773 | 76004883 | 156127 |
| 60918 | 76005962 | 76007341 | 156127 |
| 60919 | 76008266 | 76011118 | 156127 |
| 60920 | 76013904 | 76015966 | |
| 60921 | 76024408 | 76029410 | |
| 60922 | 76043640 | 76047144 | 119686, 224721 |
| 60923 | 76086702 | 76087953 | 119686 |
| 60924 | 76089526 | 76090278 | 119686 |
| 60925 | 76092548 | 76094549 | 119686 |
| 60926 | 76112311 | 76118535 | 133935, 119685, 119686 |
| 60927 | 76126698 | 76128556 | 133935, 119685, 119686 |
| 60928 | 76177371 | 76179515 | 119685 |
| 60929 | 76183893 | 76184721 | 119685 |
| 60930 | 76317668 | 76318337 | 119685 |
| 60931 | 76447744 | 76449417 | 119699, 119650 |
| 60932 | 76450870 | 76451595 | 119650 |
| 60933 | 76451824 | 76453336 | 119650 |
| 60934 | 76454891 | 76456568 | 119650 |
| 60935 | 76617824 | 76619104 | 089916 |
| 60936 | 76669788 | 76670477 | 089916 |
| 60937 | 76733631 | 76734718 | |
| 60938 | 77106640 | 77108097 | 259124 |
| 60939 | 77153560 | 77154827 | 259124 |
| 60940 | 77277806 | 77279973 | 013523 |
| 60941 | 77298937 | 77299662 | 100565 |
| 60942 | 77384219 | 77386820 | |
| 60943 | 77411322 | 77412361 | |
| 60944 | 77412690 | 77415034 | |
| 60945 | 77419489 | 77420920 | |
| 60946 | 77421968 | 77424129 | |
| 60947 | 77427588 | 77429238 | 258602 |
| 60948 | 77470338 | 77471567 | |
| 60949 | 77474199 | 77474872 | |
| 60950 | 77489456 | 77496085 | 119669 |
| 60951 | 77498458 | 77501096 | |
| 60952 | 77505838 | 77512326 | 246548 |
| 60953 | 77542446 | 77543183 | 258819, 258473 |
| 60954 | 77563047 | 77566200 | 259164, 198894 |
| 60955 | 77768342 | 77771645 | 009830 |
| 60956 | 77773335 | 77774059 | 009830 |
| 60957 | 77786517 | 77787933 | 100577, 009830 |
| 60958 | 77842404 | 77844117 | 100583, 100580 |
| 60959 | 77923121 | 77925586 | 100591, 151445 |
| 60960 | 78082030 | 78083725 | 100596 |
| 60961 | 78147769 | 78148987 | 100601 |
| 60962 | 78173428 | 78175452 | 119705, 100601 |
| 60963 | 78226541 | 78228622 | 197734, 268208, 100603, |
| | | | 119705 |
| 60964 | 78261804 | 78263024 | |
| 60965 | 78265958 | 78267391 | 063761 |
| 60966 | 78283492 | 78284106 | 063761 |
| 60967 | 79879128 | 79879818 | 021645 |
| 60968 | 80291290 | 80292309 | 021645 |
| 60969 | 80307293 | 80309351 | 021645 |
| 60970 | 80469355 | 80470455 | |
| 60971 | 81397119 | 81397628 | 100629 |
| 60972 | 81407509 | 81408816 | 100629 |
| 60973 | 81420997 | 81422013 | 165409, 100629 |
| 60974 | 81462376 | 81463214 | 165409 |
| 60975 | 81593633 | 81594598 | 258999, 165409 |
| 60976 | 81603601 | 81604723 | 258999, 165409 |
| 60977 | 81627333 | 81632082 | 258999 |
| 60978 | 81635715 | 81637619 | 258999 |
| 60979 | 81685441 | 81688817 | 165417 |
| 60980 | 81769370 | 81771184 | 140022 |
| 60981 | 81864606 | 81865492 | 140022 |
| 60982 | 81901690 | 81902262 | 140022 |
| 60983 | 81977656 | 81978275 | 071537 |
| 60984 | 81982066 | 81986598 | 071537 |
| 60985 | 81999183 | 82001759 | 071537 |
| 60986 | 82003516 | 82003967 | |
| 60987 | 82121776 | 82123044 | |
| 60988 | 82285686 | 82286196 | 259035 |
| 60989 | 82452181 | 82452976 | 259035 |
| 60990 | 86345151 | 86345685 | |
| 60991 | 86400534 | 86401336 | 258733 |
| 60992 | 86469842 | 86471072 | 258733 |
| 60993 | 87174846 | 87176186 | |
| 60994 | 87243534 | 87245393 | |
| 60995 | 87248301 | 87248857 | |
| 60996 | 87264228 | 87266136 | |
| 60997 | 87363514 | 87364796 | |
| 60998 | 88458581 | 88460295 | 054983 |
| 60999 | 88509850 | 88512661 | 258867 |
| 61000 | 88654880 | 88656297 | 100433 |
| 61001 | 88766673 | 88767713 | 100433 |
| 61002 | 88851379 | 88852846 | 042317 |
| 61003 | 88855009 | 88855574 | 042317 |
| 61004 | 88983114 | 88983785 | 070778 |
| 61005 | 88988160 | 88988552 | 070778 |
| 61006 | 89001507 | 89002013 | 070778 |
| 61007 | 89016938 | 89018124 | 258789, 070778 |
| 61008 | 89020300 | 89022166 | 070778 |
| 61009 | 89029147 | 89030280 | 100722 |
| 61010 | 89079717 | 89081465 | 100722, 165521 |
| 61011 | 89148349 | 89149364 | 165521 |
| 61012 | 89257938 | 89259663 | 165521 |
| 61013 | 89290395 | 89291792 | 165533 |
| 61014 | 89295034 | 89296820 | 165533 |
| 61015 | 89450164 | 89450741 | |
| 61016 | 89460144 | 89460862 | |
| 61017 | 89461186 | 89463721 | |
| 61018 | 89465621 | 89468407 | |
| 61019 | 89493126 | 89494342 | |
| 61020 | 89506639 | 89508109 | |
| 61021 | 89627748 | 89628404 | 053254 |
| 61022 | 89637333 | 89637988 | 053254 |
| 61023 | 89680334 | 89681104 | 053254 |
| 61024 | 89714002 | 89714530 | 053254 |
| 61025 | 89796414 | 89797311 | 053254 |
| 61026 | 89804214 | 89804652 | 053254 |
| 61027 | 89806054 | 89807152 | 053254 |
| 61028 | 89817674 | 89818726 | 258752, 053254 |
| 61029 | 89834676 | 89835982 | 053254 |
| 61030 | 89844421 | 89844999 | 053254 |
| 61031 | 89874320 | 89875510 | 258380, 053254 |
| 61032 | 89881867 | 89885133 | 258920, 053254, 259053 |
| 61033 | 89894715 | 89897872 | 053254, 259053 |
| 61034 | 89924974 | 89925748 | 053254, 259053 |
| 61035 | 89940249 | 89941210 | 053254, 259053 |
| 61036 | 89959742 | 89960722 | 053254, 259053 |
| 61037 | 89966511 | 89967110 | 053254, 259053 |
| 61038 | 89973207 | 89975150 | 053254, 259053 |
| 61039 | 90010915 | 90011587 | 053254, 259053 |
| 61040 | 90083841 | 90086241 | 053254, 259053 |
| 61041 | 90107684 | 90108836 | 259053, 258792 |
| 61042 | 90125499 | 90127427 | 259053 |
| 61043 | 90167842 | 90168528 | 259053 |
| 61044 | 90212240 | 90212933 | 259053 |
| 61045 | 90377055 | 90377988 | 259053, 140025 |
| 61046 | 90390523 | 90391042 | 259053, 140025 |
| 61047 | 90420631 | 90423142 | 042088, 259053, 140025 |
| 61048 | 90553799 | 90554358 | 152315 |
| 61049 | 90722509 | 90723936 | 100764 |
| 61050 | 90744502 | 90745380 | 119720 |
| 61051 | 90797172 | 90798864 | 119720 |
| 61052 | 90835572 | 90836192 | |
| 61053 | 90848096 | 90851024 | |
| 61054 | 90862541 | 90865033 | 198668 |
| 61055 | 90875352 | 90875970 | |
| 61056 | 91112228 | 91112859 | 259163, 165914 |
| 61057 | 91163698 | 91164531 | 165914, 258716 |
| 61058 | 91224408 | 91225827 | 165914 |
| 61059 | 91246720 | 91247832 | 165914 |
| 61060 | 91274006 | 91274875 | 165914 |
| 61061 | 91278289 | 91278809 | 165914 |
| 61062 | 91281385 | 91281975 | 165914 |
| 61063 | 91287068 | 91287785 | |
| 61064 | 91294279 | 91295513 | 258884 |
| 61065 | 91337876 | 91338568 | 100784 |
| 61066 | 91436399 | 91436898 | 100784 |
| 61067 | 91488578 | 91489598 | 100784 |
| 61068 | 91526011 | 91527620 | 133943, 100784 |
| 61069 | 91564918 | 91565762 | 133943 |
| 61070 | 91579974 | 91582168 | 133943 |
| 61071 | 91615716 | 91616310 | 133943 |
| 61072 | 91622594 | 91623110 | 133943 |
| 61073 | 91630887 | 91631549 | 133943 |
| 61074 | 91883782 | 91885548 | 258798, 015133 |
| 61075 | 91974439 | 91978066 | 100796 |
| 61076 | 91985768 | 91986549 | |
| 61077 | 92040261 | 92041501 | 268657 |
| 61078 | 92221879 | 92222662 | 133962 |
| 61079 | 92246567 | 92247450 | 133962, 165929 |
| 61080 | 92354922 | 92356622 | 140092 |
| 61081 | 92363565 | 92364174 | 140092 |
| 61082 | 92424132 | 92424624 | |
| 61083 | 92462855 | 92464632 | 100815 |
| 61084 | 92476910 | 92477413 | 100815 |
| 61085 | 92505325 | 92507432 | 100815 |
| 61086 | 92571840 | 92573526 | 066427 |
| 61087 | 92587471 | 92589481 | 165934, 183648 |
| 61088 | 92938206 | 92938784 | 140090 |
| 61089 | 92938949 | 92940880 | 140090 |
| 61090 | 92947134 | 92948042 | 140090 |
| 61091 | 92969464 | 92971962 | |
| 61092 | 92977226 | 92978318 | |
| 61093 | 92979402 | 92981869 | 100599 |
| 61094 | 92995572 | 92996526 | 100599 |
| 61095 | 93004709 | 93007531 | 100599 |
| 61096 | 93160283 | 93160825 | |
| 61097 | 93190669 | 93191545 | 100600 |
| 61098 | 93213926 | 93215423 | 100600 |
| 61099 | 93259543 | 93261662 | 066455 |
| 61100 | 93264294 | 93265160 | 066455 |
| 61101 | 93344116 | 93344986 | |
| 61102 | 93439493 | 93441653 | 100605 |
| 61103 | 93450540 | 93453922 | 100605 |
| 61104 | 93459097 | 93460224 | 100605 |
| 61105 | 93483533 | 93485003 | 100605 |
| 61106 | 93501525 | 93504342 | 100605 |
| 61107 | 93508175 | 93511955 | 100605 |
| 61108 | 93521908 | 93524319 | 100605 |
| 61109 | 93579060 | 93580354 | 100605 |
| 61110 | 93580377 | 93583276 | 100605 |
| 61111 | 93598692 | 93599349 | |
| 61112 | 93601676 | 93602767 | |
| 61113 | 93617342 | 93617953 | |
| 61114 | 93650191 | 93652457 | 259066, 153485, 165943 |
| 61115 | 93672777 | 93674347 | 012963, 170270, 259066 |
| 61116 | 93798278 | 93800568 | 133958, 011114 |
| 61117 | 94424717 | 94427371 | 100628 |
| 61118 | 94492497 | 94493463 | 089723 |
| 61119 | 94743485 | 94744045 | 119698 |
| 61120 | 94753112 | 94754028 | 140093 |
| 61121 | 94754507 | 94760995 | 140093 |
| 61122 | 94761265 | 94763275 | |
| 61123 | 94772064 | 94772901 | 170099 |
| 61124 | 94778480 | 94781502 | 170099 |
| 61125 | 94783497 | 94784599 | 170099 |
| 61126 | 94786274 | 94790123 | 170099 |
| 61127 | 94796777 | 94798152 | |
| 61128 | 94803581 | 94804197 | |
| 61129 | 94806745 | 94810014 | |
| 61130 | 94811607 | 94812502 | |
| 61131 | 94821041 | 94822431 | |
| 61132 | 94830349 | 94830964 | 258597 |
| 61133 | 94842479 | 94844918 | 197249 |
| 61134 | 94845980 | 94847954 | 197249 |
| 61135 | 94850584 | 94859245 | 197249 |
| 61136 | 94859295 | 94862853 | |
| 61137 | 94877531 | 94878473 | |
| 61138 | 94918826 | 94919406 | 186910, 256357 |
| 61139 | 94929289 | 94930027 | 170054, 256357 |
| 61140 | 94947037 | 94948663 | |
| 61141 | 94952195 | 94953424 | |
| 61142 | 94967985 | 94968985 | 165953 |
| 61143 | 95025951 | 95028700 | 100665, 188488 |
| 61144 | 95035754 | 95036453 | 100665, 188488 |
| 61145 | 95047432 | 95048995 | 188488 |
| 61146 | 95049106 | 95049665 | 188488 |
| 61147 | 95059671 | 95061536 | 273259 |
| 61148 | 95063417 | 95069047 | 273259 |
| 61149 | 95075739 | 95089142 | 196136, 273259 |
| 61150 | 95146209 | 95148172 | |
| 61151 | 95622310 | 95625011 | 235706, 100697 |
| 61152 | 95647303 | 95648104 | |
| 61153 | 95649391 | 95650497 | 165959 |
| 61154 | 95688492 | 95689953 | 165959 |
| 61155 | 95703353 | 95704367 | 165959 |
| 61156 | 95714586 | 95716176 | 165959 |
| 61157 | 95727655 | 95728926 | 165959 |
| 61158 | 95730401 | 95732949 | 165959 |
| 61159 | 95757371 | 95759007 | 165959 |
| 61160 | 95760815 | 95762337 | 165959 |
| 61161 | 95766408 | 95768243 | 165959 |
| 61162 | 95773268 | 95774736 | 165959 |
| 61163 | 95775377 | 95776344 | 165959 |
| 61164 | 95779628 | 95782246 | 165959 |
| 61165 | 95784437 | 95787055 | 165959 |
| 61166 | 95805319 | 95808039 | |
| 61167 | 95831052 | 95832156 | |
| 61168 | 95865127 | 95865840 | |
| 61169 | 95900431 | 95900996 | 176438 |
| 61170 | 95910279 | 95911066 | 176438 |
| 61171 | 95949023 | 95949935 | |
| 61172 | 95955426 | 95956065 | |
| 61173 | 95973567 | 95974636 | |
| 61174 | 95982257 | 95983848 | 258572 |
| 61175 | 95989036 | 95994324 | |
| 61176 | 95999711 | 96002945 | 182512, 252481, 247092 |
| 61177 | 96003548 | 96004404 | 182512 |
| 61178 | 96013829 | 96014524 | |
| 61179 | 96016447 | 96017211 | |
| 61180 | 96561504 | 96563123 | |
| 61181 | 96566685 | 96567580 | |
| 61182 | 96570272 | 96572941 | |
| 61183 | 96633660 | 96634217 | |
| 61184 | 96660883 | 96662394 | |
| 61185 | 96670880 | 96671415 | 258691, 168398 |
| 61186 | 96734292 | 96736200 | 100739, 258793 |
| 61187 | 96739368 | 96739809 | |
| 61188 | 96741613 | 96742493 | 258729 |
| 61189 | 96750328 | 96751321 | 066739 |
| 61190 | 96768471 | 96769047 | 066739 |
| 61191 | 96775392 | 96776125 | 066739 |
| 61192 | 96828309 | 96830923 | 100744, 066739 |
| 61193 | 96857356 | 96859359 | 140057 |
| 61194 | 96928065 | 96929271 | 140057 |
| 61195 | 96934581 | 96935607 | 140057 |
| 61196 | 96967202 | 96969963 | 090060, 260806 |
| 61197 | 96978200 | 96979617 | 090060 |
| 61198 | 97004233 | 97005767 | 090060 |
| 61199 | 97263356 | 97264322 | 100749 |
| 61200 | 97736411 | 97737384 | |
| 61201 | 99782280 | 99783455 | |
| 61202 | 99851086 | 99851854 | |
| 61203 | 99946182 | 99948571 | 090061, 183576 |
| 61204 | 99984563 | 99985819 | 090061, 205476 |
| 61205 | 100018248 | 100019799 | 205476 |
| 61206 | 100020524 | 100022003 | 205476 |
| 61207 | 100027464 | 100028083 | 205476 |
| 61208 | 100031218 | 100032126 | 205476 |
| 61209 | 100035424 | 100039267 | 205476 |
| 61210 | 100040347 | 100042346 | 205476 |
| 61211 | 100056985 | 100057543 | 205476 |
| 61212 | 100060758 | 100061924 | 205476 |
| 61213 | 100062933 | 100063633 | 205476 |
| 61214 | 100067575 | 100069333 | 205476 |
| 61215 | 100070536 | 100072291 | 247970 |
| 61216 | 100137999 | 100138607 | 182218 |
| 61217 | 100148966 | 100150958 | 036530 |
| 61218 | 100222433 | 100223024 | 066629 |
| 61219 | 100250660 | 100251244 | 066629 |
| 61220 | 100259426 | 100260316 | 066629 |
| 61221 | 100410158 | 100411239 | |
| 61222 | 100411996 | 100412764 | |
| 61223 | 100414046 | 100414430 | |
| 61224 | 100437436 | 100439086 | 196405 |
| 61225 | 100484717 | 100485413 | 196405 |
| 61226 | 100495551 | 100499529 | 196405 |
| 61227 | 100508554 | 100509284 | 196405 |
| 61228 | 100658454 | 100660340 | |
| 61229 | 100670614 | 100671333 | |
| 61230 | 100679938 | 100681385 | 258982 |
| 61231 | 100703892 | 100707685 | 100811, 258982 |
| 61232 | 100751435 | 100752617 | 259052 |
| 61233 | 100771778 | 100773753 | 197119 |
| 61234 | 100786669 | 100787251 | |
| 61235 | 100789063 | 100792859 | 263519, 140107 |
| 61236 | 100806717 | 100809053 | 258521, 258666, 140105 |
| 61237 | 100841394 | 100843607 | 176473, 140105 |
| 61238 | 100844027 | 100844604 | 176473 |
| 61239 | 100855796 | 100856346 | 176473 |
| 61240 | 100863834 | 100865186 | 176473 |
| 61241 | 100872234 | 100873321 | 258620, 176473 |
| 61242 | 100904832 | 100907395 | 176473 |
| 61243 | 101000343 | 101001039 | |
| 61244 | 101083222 | 101083681 | |
| 61245 | 101114688 | 101116268 | |
| 61246 | 101131408 | 101132393 | 196273 |
| 61247 | 101800669 | 101802119 | 258460 |
| 61248 | 101871564 | 101872361 | 259023 |
| 61249 | 101878949 | 101879492 | |
| 61250 | 101910310 | 101910875 | 259082 |
| 61251 | 101941895 | 101943975 | |
| 61252 | 101946438 | 101948122 | |
| 61253 | 102197435 | 102198788 | 258404, 258512 |
| 61254 | 102228033 | 102228953 | 078304 |
| 61255 | 102275376 | 102277619 | 259088, 078304 |
| 61256 | 102413594 | 102416113 | 271780 |
| 61257 | 102430572 | 102432444 | 197102 |
| 61258 | 102438064 | 102440009 | 197102 |
| 61259 | 102446242 | 102447658 | 197102 |
| 61260 | 102500618 | 102500964 | 197102 |
| 61261 | 102552356 | 102554882 | 080824 |
| 61262 | 102605370 | 102607343 | 140153, 080824 |
| 61263 | 102656925 | 102657874 | 140153 |
| 61264 | 102746003 | 102746531 | 080823 |
| 61265 | 102770769 | 102771843 | 080823 |
| 61266 | 102780070 | 102780967 | |
| 61267 | 102782867 | 102784354 | 022976 |
| 61268 | 102785333 | 102787273 | 022976 |
| 61269 | 102828317 | 102830436 | 196663, 100865 |
| 61270 | 102946125 | 102946726 | 196663 |
| 61271 | 102974784 | 102976221 | 156381 |
| 61272 | 102982738 | 102984124 | |
| 61273 | 102990547 | 102992142 | |
| 61274 | 103020862 | 103022245 | 259230 |
| 61275 | 103057536 | 103060400 | 089902 |
| 61276 | 103092196 | 103092709 | 089902 |
| 61277 | 103121071 | 103122202 | 089902 |
| 61278 | 103149346 | 103151310 | 089902 |
| 61279 | 103162651 | 103163312 | 089902 |
| 61280 | 103242448 | 103245308 | 131323 |
| 61281 | 103261344 | 103261971 | 131323 |
| 61282 | 103315691 | 103316417 | 131323 |
| 61283 | 103337140 | 103338567 | 131323 |
| 61284 | 103339765 | 103340508 | 131323 |
| 61285 | 103359669 | 103360353 | 131323 |
| 61286 | 103388605 | 103389918 | 166126 |
| 61287 | 103406562 | 103406993 | 198752 |
| 61288 | 103470420 | 103471094 | 198752 |
| 61289 | 103517012 | 103517734 | 198752 |
| 61290 | 103522479 | 103524917 | 198752 |
| 61291 | 103540405 | 103542539 | |
| 61292 | 103557460 | 103558920 | |
| 61293 | 103560265 | 103562009 | 259444 |
| 61294 | 103564526 | 103567272 | 205436, 259444 |
| 61295 | 103569729 | 103572540 | 205436 |
| 61296 | 103573535 | 103579591 | 205436 |
| 61297 | 103581936 | 103582679 | |
| 61298 | 103587456 | 103590508 | 185215, 259717 |
| 61299 | 103621284 | 103621825 | |
| 61300 | 103627198 | 103627988 | |
| 61301 | 103664230 | 103666124 | |
| 61302 | 103671284 | 103676209 | 270705 |
| 61303 | 103679269 | 103680245 | |
| 61304 | 103709710 | 103712897 | |
| 61305 | 103726119 | 103727518 | |
| 61306 | 103750540 | 103751810 | |
| 61307 | 103762374 | 103762865 | |
| 61308 | 103780862 | 103782777 | |
| 61309 | 103796001 | 103796794 | |
| 61310 | 103798548 | 103802065 | 100664, 259775 |
| 61311 | 103851302 | 103853658 | 075413 |
| 61312 | 103994459 | 103996435 | 166166, 260285 |
| 61313 | 104018308 | 104019161 | |
| 61314 | 104024354 | 104024926 | 166170, 258851 |
| 61315 | 104027831 | 104029886 | 126214, 256053, 256500, |
| | | | 166170, 258851 |
| 61316 | 104043009 | 104043443 | 126214, 256053, 256500 |
| 61317 | 104094111 | 104096949 | 126214, 256500 |
| 61318 | 104181465 | 104182977 | 100711, 126215 |
| 61319 | 104211659 | 104212196 | 088808 |
| 61320 | 104312915 | 104315265 | 258735, 088808 |
| 61321 | 104338046 | 104339488 | 258534 |
| 61322 | 104345571 | 104348552 | 258534 |
| 61323 | 104354459 | 104354981 | |
| 61324 | 104355428 | 104359099 | |
| 61325 | 104360153 | 104361576 | |
| 61326 | 104363256 | 104364158 | |
| 61327 | 104387075 | 104388099 | 156411 |
| 61328 | 104544163 | 104544816 | |
| 61329 | 104554196 | 104555198 | 166183 |
| 61330 | 104582611 | 104583940 | 207568 |
| 61331 | 104747189 | 104747705 | 258913 |
| 61332 | 105120370 | 105120806 | 258736 |
| 61333 | 105146053 | 105148490 | 256050 |
| 61334 | 105153825 | 105157270 | 203485, 258858 |
| 61335 | 105160402 | 105161593 | 203485 |
| 61336 | 105189799 | 105192143 | 185100 |
| 61337 | 105192537 | 105193836 | 185100 |
| 61338 | 105198086 | 105200680 | 185100 |
| 61339 | 105200901 | 105203543 | 185100 |
| 61340 | 105215336 | 105216252 | |
| 61341 | 105218643 | 105220220 | 184990 |
| 61342 | 105255518 | 105256301 | 142208 |
| 61343 | 105258296 | 105258929 | 142208 |
| 61344 | 105260734 | 105263112 | 142208 |
| 61345 | 105266381 | 105268027 | 179627 |
| 61346 | 105281428 | 105283472 | |
| 61347 | 105292379 | 105294305 | |
| 61348 | 105330163 | 105333114 | 099814 |
| 61349 | 105338876 | 105339610 | 099814 |
| 61350 | 105399527 | 105400103 | 166428 |
| 61351 | 105442297 | 105443031 | 185567 |
| 61352 | 105444055 | 105444772 | 185567 |
| 61353 | 105446304 | 105447172 | |
| 61354 | 105451713 | 105453196 | 140104 |
| 61355 | 105486513 | 105488757 | 170779 |
| 61356 | 105511908 | 105512574 | |
| 61357 | 105553005 | 105555135 | |
| 61358 | 105556724 | 105560493 | 257556 |
| 61359 | 105646876 | 105647862 | 257622, 183828 |
| 61360 | 105670779 | 105671406 | |
| 61361 | 105714471 | 105715537 | 184887, 185024 |
| 61362 | 105765990 | 105768026 | 179364, 185024 |
| 61363 | 105779957 | 105781954 | 185024, 179364 |
| 61364 | 105804286 | 105804869 | 179364 |
| 61365 | 105863845 | 105865836 | 226174, 179364 |
| 61366 | 105881278 | 105881669 | 226174 |
| 61367 | 105885357 | 105887384 | 182979, 251602, 226174 |
| 61368 | 105897853 | 105898561 | 182979, 226174 |
| 61369 | 105899480 | 105900125 | 182979, 226174 |
| 61370 | 105941124 | 105943206 | 182809 |
| 61371 | 105947123 | 105948556 | |
| 61372 | 105955842 | 105958939 | 185347, 257341 |
| 61373 | 105994203 | 105995295 | 184986 |
| 61374 | 107251818 | 107253961 | |

The locations of ODSCs on Chromosome 15 are shown in Table 57.

TABLE 57
ODSC Locations on Chromosome 15

| SEQ ID NO. | Start | End | Overlapping Gene(s) |
|---|---|---|---|
| 61375 | 22832929 | 22834332 | 153575 |
| 61376 | 22891634 | 22894164 | 068793 |
| 61377 | 23033218 | 23034934 | 140157 |
| 61378 | 23085523 | 23087423 | 170113 |
| 61379 | 25200014 | 25202025 | 214265, 273173, 128739 |
| 61380 | 25682717 | 25685233 | 114062 |
| 61381 | 26327001 | 26328372 | |
| 61382 | 28282768 | 28284124 | 104044 |
| 61383 | 28327727 | 28328668 | 104044 |
| 61384 | 29561142 | 29562895 | 185115, 104059 |
| 61385 | 29731187 | 29732041 | 104059 |
| 61386 | 29762035 | 29764109 | 104059 |
| 61387 | 29961475 | 29968684 | 256802 |
| 61388 | 29982987 | 29983903 | |
| 61389 | 29989375 | 29991564 | |
| 61390 | 30004276 | 30005448 | 104067 |
| 61391 | 30027102 | 30029665 | 104067 |
| 61392 | 30048749 | 30049483 | 104067 |
| 61393 | 30112909 | 30115930 | 259523, 104067 |
| 61394 | 30216857 | 30218480 | 104067 |
| 61395 | 30234319 | 30235186 | 104067 |
| 61396 | 30260266 | 30261513 | 104067 |
| 61397 | 30330904 | 30331895 | 259647 |
| 61398 | 30357272 | 30358119 | 229389 |
| 61399 | 31195534 | 31197064 | 198690 |
| 61400 | 31248382 | 31248888 | 212526, 166912 |
| 61401 | 31281049 | 31285071 | 166912 |
| 61402 | 31361492 | 31362029 | 134160 |
| 61403 | 31391440 | 31392189 | 134160 |
| 61404 | 31449441 | 31450576 | 134160 |
| 61405 | 31451112 | 31452678 | 134160 |
| 61406 | 31455875 | 31457090 | |
| 61407 | 31506794 | 31509502 | 259448 |
| 61408 | 31528346 | 31529163 | |
| 61409 | 31554721 | 31558493 | |
| 61410 | 31571525 | 31572728 | |
| 61411 | 31588389 | 31590108 | |
| 61412 | 31617259 | 31621668 | 169926 |
| 61413 | 31636468 | 31637487 | 169926 |
| 61414 | 31637947 | 31638825 | 169926 |
| 61415 | 31645366 | 31646576 | 169926 |
| 61416 | 31657197 | 31659130 | 169926 |
| 61417 | 31683560 | 31684779 | 169926 |
| 61418 | 31689027 | 31689930 | 169926 |
| 61419 | 31732522 | 31733888 | |
| 61420 | 31735828 | 31736695 | |
| 61421 | 31779838 | 31781618 | 169918 |
| 61422 | 32951506 | 32952168 | 166922, 241818 |
| 61423 | 32968879 | 32970733 | 166922 |
| 61424 | 32973820 | 32977127 | 166922 |
| 61425 | 33492428 | 33493300 | |
| 61426 | 33493447 | 33494462 | |
| 61427 | 33585668 | 33586424 | |
| 61428 | 34329992 | 34331994 | 169857, 184984 |
| 61429 | 34393156 | 34394954 | 182405, 134153 |
| 61430 | 34446992 | 34448032 | 134152 |
| 61431 | 34501430 | 34502792 | 134152 |
| 61432 | 34516063 | 34518035 | 128463 |
| 61433 | 34629462 | 34630629 | 140199 |
| 61434 | 34634545 | 34635662 | 184507, 182117 |
| 61435 | 34658949 | 34660702 | 176454 |
| 61436 | 34806252 | 34807666 | 175265 |
| 61437 | 34874875 | 34876996 | 175265, 215252 |
| 61438 | 34880048 | 34880940 | 175265, 215252 |
| 61439 | 35011887 | 35012458 | |
| 61440 | 35260908 | 35262364 | 021776 |
| 61441 | 35279044 | 35281444 | 198146 |
| 61442 | 35400635 | 35401295 | 259336 |
| 61443 | 35410784 | 35411634 | 259336 |
| 61444 | 35433752 | 35434459 | 259336 |
| 61445 | 35501831 | 35502929 | 259516 |
| 61446 | 35584744 | 35586601 | 134146 |
| 61447 | 35593195 | 35593873 | 134146 |
| 61448 | 35596899 | 35600079 | 134146 |
| 61449 | 35600540 | 35602971 | 134146 |
| 61450 | 35604499 | 35604958 | 134146 |
| 61451 | 35742474 | 35743263 | 134146 |
| 61452 | 35837019 | 35839344 | 248079, 134146 |
| 61453 | 35848995 | 35850688 | 248079 |
| 61454 | 35922406 | 35923494 | 248079 |
| 61455 | 35989703 | 35990365 | 248079 |
| 61456 | 36045451 | 36046549 | 248079 |
| 61457 | 36166425 | 36166855 | |
| 61458 | 36469916 | 36470694 | 259639 |
| 61459 | 36720137 | 36721563 | |
| 61460 | 36798913 | 36799447 | |
| 61461 | 36871283 | 36872530 | 186073 |
| 61462 | 37076410 | 37078067 | 186073 |
| 61463 | 37090488 | 37090966 | 223518, 186073 |
| 61464 | 37097467 | 37098261 | 186073, 223518 |
| 61465 | 37100231 | 37101549 | 186073, 223518 |
| 61466 | 37106257 | 37106788 | 223518 |
| 61467 | 37106837 | 37107283 | 223518 |
| 61468 | 37301494 | 37302250 | 134138 |
| 61469 | 37390546 | 37394483 | 259460, 134138 |
| 61470 | 37508545 | 37512270 | |
| 61471 | 38050441 | 38051509 | |
| 61472 | 38056484 | 38057879 | |
| 61473 | 38068962 | 38069872 | |
| 61474 | 38079888 | 38083438 | |
| 61475 | 38116796 | 38117947 | |
| 61476 | 38152964 | 38153682 | |
| 61477 | 38164486 | 38165925 | |
| 61478 | 38177657 | 38178479 | |
| 61479 | 38192440 | 38194272 | |
| 61480 | 38342433 | 38343164 | 259225 |
| 61481 | 38364772 | 38365964 | 236914 |
| 61482 | 38543436 | 38545675 | 166068 |
| 61483 | 38746031 | 38747176 | 171262 |
| 61484 | 38856133 | 38857075 | 172575 |
| 61485 | 39160459 | 39161440 | 259345 |
| 61486 | 39166567 | 39167703 | 259345 |
| 61487 | 39169580 | 39170165 | 259345 |
| 61488 | 39172072 | 39172359 | 259345 |
| 61489 | 39186536 | 39187151 | 259345 |
| 61490 | 39419212 | 39421566 | 259345 |
| 61491 | 39445845 | 39446840 | 259345 |
| 61492 | 39481031 | 39481474 | 259345, 259450 |
| 61493 | 39565525 | 39566843 | 259345 |
| 61494 | 39645299 | 39646070 | 259345 |
| 61495 | 39650323 | 39653927 | 259345 |
| 61496 | 39655022 | 39657306 | 259345 |
| 61497 | 39871623 | 39874302 | 137801 |
| 61498 | 40074796 | 40075848 | 150667, 259580, 261136 |
| 61499 | 40079539 | 40080157 | 259580 |
| 61500 | 40096025 | 40096670 | 166073 |
| 61501 | 40120705 | 40121213 | 166073 |
| 61502 | 40152344 | 40153408 | 166073 |
| 61503 | 40177509 | 40177953 | 166073 |
| 61504 | 40210672 | 40214000 | 246863, 166073 |
| 61505 | 40225115 | 40227285 | 128829 |
| 61506 | 40227600 | 40228778 | 128829 |
| 61507 | 40268582 | 40269301 | 128829 |
| 61508 | 40294121 | 40296411 | 128829 |
| 61509 | 40302861 | 40303295 | 128829 |
| 61510 | 40330133 | 40333615 | 248508, 140319 |
| 61511 | 40347136 | 40347880 | 248508 |
| 61512 | 40361033 | 40362325 | |
| 61513 | 40380516 | 40381749 | 259409, 104081 |
| 61514 | 40384459 | 40385497 | 104081 |
| 61515 | 40390013 | 40394020 | 104081 |
| 61516 | 40394573 | 40396409 | 104081 |
| 61517 | 40400722 | 40401587 | 104081 |
| 61518 | 40452672 | 40454103 | 156970 |
| 61519 | 40574103 | 40575255 | 230778, 137841 |
| 61520 | 40583350 | 40583681 | 137841 |
| 61521 | 40615447 | 40616914 | |
| 61522 | 40630759 | 40633513 | 188549 |
| 61523 | 40659122 | 40659557 | 140323 |
| 61524 | 40659926 | 40660784 | 140323 |
| 61525 | 40671929 | 40672569 | |
| 61526 | 40674366 | 40675953 | 128944 |
| 61527 | 40704098 | 40705406 | 128928 |
| 61528 | 40732498 | 40733116 | 140320 |
| 61529 | 40733152 | 40734806 | 140320 |
| 61530 | 40762901 | 40764243 | 169105 |
| 61531 | 40785053 | 40786576 | 259536 |
| 61532 | 40803228 | 40804774 | 259536 |
| 61533 | 40841943 | 40842526 | 128891, 259536 |
| 61534 | 40856164 | 40857475 | 128891 |
| 61535 | 40861111 | 40862516 | 166133 |
| 61536 | 40885725 | 40886858 | 137812 |
| 61537 | 40941971 | 40943291 | 137812 |
| 61538 | 40986267 | 40987547 | 051180, 245849 |
| 61539 | 41034936 | 41039116 | 137824 |
| 61540 | 41046297 | 41048138 | 137824 |
| 61541 | 41055383 | 41057950 | 137880 |
| 61542 | 41063246 | 41063843 | 188277, 104129 |
| 61543 | 41085302 | 41086565 | 104129 |
| 61544 | 41098617 | 41100466 | 166140, 104129 |
| 61545 | 41137761 | 41139755 | 166145 |
| 61546 | 41185718 | 41187508 | 104142 |
| 61547 | 41198156 | 41199672 | 251161 |
| 61548 | 41201654 | 41202712 | |
| 61549 | 41232897 | 41234953 | |
| 61550 | 41241880 | 41242742 | |
| 61551 | 41244284 | 41246430 | 128965 |
| 61552 | 41293364 | 41294417 | 128908, 259617 |
| 61553 | 41335394 | 41336131 | 128908 |
| 61554 | 41406826 | 41409736 | 128908 |
| 61555 | 41453043 | 41453965 | |
| 61556 | 41522561 | 41524466 | 187446, 178997 |
| 61557 | 41525502 | 41525998 | 187446 |
| 61558 | 41533360 | 41534533 | 187446 |
| 61559 | 41541884 | 41542488 | 187446 |
| 61560 | 41547803 | 41549969 | 187446 |
| 61561 | 41550545 | 41552799 | 187446 |
| 61562 | 41568794 | 41569558 | 187446 |
| 61563 | 41571257 | 41571713 | 187446 |
| 61564 | 41575160 | 41577524 | 247556 |
| 61565 | 41623884 | 41625384 | 137804, 104147 |
| 61566 | 41660558 | 41660992 | 137804 |
| 61567 | 41693626 | 41695418 | 137806 |
| 61568 | 41708335 | 41710542 | 137815 |
| 61569 | 41787400 | 41789178 | 137825 |
| 61570 | 41799989 | 41800670 | 062524 |
| 61571 | 41835538 | 41836717 | 103932 |
| 61572 | 41850849 | 41852220 | 092445 |
| 61573 | 41912813 | 41914578 | 174197 |
| 61574 | 41953355 | 41954407 | 174197 |
| 61575 | 42065990 | 42067609 | 137802 |
| 61576 | 42082583 | 42083643 | 137802 |
| 61577 | 42091218 | 42091948 | 137802 |
| 61578 | 42119677 | 42121032 | 168970, 243789, 243708, |
| | | | 137802, 250379 |
| 61579 | 42176534 | 42177191 | 137877 |
| 61580 | 42218478 | 42219590 | 259883, 103966 |
| 61581 | 42220005 | 42223320 | 259883, 103966 |
| 61582 | 42235562 | 42237160 | 103966 |
| 61583 | 42251733 | 42255067 | 103966 |
| 61584 | 42261903 | 42262574 | 103966 |
| 61585 | 42263679 | 42265494 | 246740, 103966 |
| 61586 | 42300567 | 42304948 | 257797, 188089 |
| 61587 | 42348607 | 42351085 | |
| 61588 | 42352964 | 42353482 | |
| 61589 | 42376400 | 42377683 | 159337 |
| 61590 | 42499675 | 42500724 | 261002, 166887 |
| 61591 | 42564391 | 42568259 | 214013, 103978 |
| 61592 | 42578316 | 42579500 | 214013 |
| 61593 | 42698240 | 42698798 | 258461, 092529 |
| 61594 | 42782363 | 42784204 | 092531, 103994 |
| 61595 | 42786622 | 42788542 | 092531 |
| 61596 | 42791373 | 42791892 | 092531 |
| 61597 | 42840245 | 42841732 | 137814, 180979 |
| 61598 | 42867099 | 42868288 | 159433 |
| 61599 | 42883582 | 42884171 | 159433 |
| 61600 | 42999270 | 42999678 | 159433 |
| 61601 | 43011040 | 43011697 | 159433 |
| 61602 | 43028489 | 43030203 | 140326 |
| 61603 | 43212129 | 43213872 | 128881 |
| 61604 | 43378109 | 43378557 | 159459 |
| 61605 | 43397501 | 43399027 | 166947, 159459 |
| 61606 | 43424937 | 43426836 | 137842, 166947 |
| 61607 | 43477378 | 43478858 | 166946, 166947 |
| 61608 | 43566327 | 43566652 | |
| 61609 | 43621507 | 43623627 | 168803, 168806 |
| 61610 | 43662433 | 43664100 | 140265, 137822 |
| 61611 | 43732977 | 43733653 | 067369 |
| 61612 | 43784601 | 43786061 | 067369 |
| 61613 | 43802357 | 43803354 | 166963, 067369 |
| 61614 | 44037957 | 44039465 | 167004, 205771 |
| 61615 | 44041768 | 44042499 | 167004 |
| 61616 | 44069264 | 44070395 | 140264, 128886, 262560 |
| 61617 | 44083407 | 44085817 | 140264, 262560 |
| 61618 | 44091916 | 44093575 | 184716, 140264, 242028 |
| 61619 | 44115749 | 44117129 | 140259 |
| 61620 | 44579859 | 44581313 | 166734 |
| 61621 | 44718930 | 44720880 | 137770, 259595 |
| 61622 | 44827931 | 44830569 | 104131, 179523 |
| 61623 | 44954287 | 44956678 | 104133 |
| 61624 | 45002805 | 45005722 | 166710, 229474 |
| 61625 | 45018832 | 45022402 | 185880 |
| 61626 | 45075822 | 45077043 | |
| 61627 | 45103580 | 45105079 | |
| 61628 | 45174667 | 45175180 | 259479 |
| 61629 | 45315118 | 45316620 | 140263 |
| 61630 | 45375366 | 45376022 | |
| 61631 | 45383275 | 45383814 | |
| 61632 | 45423520 | 45424364 | 137857 |
| 61633 | 45454519 | 45455945 | 137857, 259539 |
| 61634 | 45458420 | 45460013 | 138606, 259539 |
| 61635 | 45478735 | 45480278 | 138606, 259519 |
| 61636 | 45490464 | 45493801 | 259932, 260035, 138606, |
| | | | 259519 |
| 61637 | 45570710 | 45572403 | 259520 |
| 61638 | 45635018 | 45638107 | |
| 61639 | 45659407 | 45660130 | 171766 |
| 61640 | 45667699 | 45668163 | 171766 |
| 61641 | 45669333 | 45672007 | 171766 |
| 61642 | 45678544 | 45680216 | 171766 |
| 61643 | 45693336 | 45696728 | 171763, 171766 |
| 61644 | 45722246 | 45723179 | 166920, 259342 |
| 61645 | 45736827 | 45738696 | 252285, 166920 |
| 61646 | 45740018 | 45742059 | 259354, 166920 |
| 61647 | 45748464 | 45749227 | 259354 |
| 61648 | 45813775 | 45815392 | 104154, 179362 |
| 61649 | 45878248 | 45880833 | 260170, 104164, 179362 |
| 61650 | 45926264 | 45935452 | 260170, 137767 |
| 61651 | 45937579 | 45938228 | 260170, 137767 |
| 61652 | 45940093 | 45941948 | 260170, 137767 |
| 61653 | 45951213 | 45953636 | 260170, 137767 |
| 61654 | 45996459 | 45998555 | 259200 |
| 61655 | 46211983 | 46212734 | 259200 |
| 61656 | 47416530 | 47417177 | |
| 61657 | 47585969 | 47586402 | 137872 |
| 61658 | 48104302 | 48105219 | 259572 |
| 61659 | 48622925 | 48624657 | 128951, 259488 |
| 61660 | 48937768 | 48938655 | 259705, 166147 |
| 61661 | 48975103 | 48975636 | |
| 61662 | 49073549 | 49074297 | 103995 |
| 61663 | 49076980 | 49078042 | 103995 |
| 61664 | 49086631 | 49087586 | 103995 |
| 61665 | 49147778 | 49148553 | 185634 |
| 61666 | 49169728 | 49171100 | 255302, 235883, 185634 |
| 61667 | 49267795 | 49268505 | |
| 61668 | 49337690 | 49339440 | 138593 |
| 61669 | 49446635 | 49448600 | 156958, 259467, 166200 |
| 61670 | 49462040 | 49463356 | 156958 |
| 61671 | 49784320 | 49785578 | 166262 |
| 61672 | 49912305 | 49914407 | 104047, 166262 |
| 61673 | 50147958 | 50148469 | |
| 61674 | 50238846 | 50239410 | 104043 |
| 61675 | 50336979 | 50337664 | 104043 |
| 61676 | 50426189 | 50429200 | 104043 |
| 61677 | 50436893 | 50439316 | 104043 |
| 61678 | 50455027 | 50459765 | 104043 |
| 61679 | 50460336 | 50461223 | 104043 |
| 61680 | 50473263 | 50476237 | 140284, 104043 |
| 61681 | 50502617 | 50503458 | 140284 |
| 61682 | 50518028 | 50518595 | 140284 |
| 61683 | 50519069 | 50519719 | 140284 |
| 61684 | 50530106 | 50530884 | |
| 61685 | 50539638 | 50540460 | 140287 |
| 61686 | 50540860 | 50541712 | 140287 |
| 61687 | 50644634 | 50648467 | 244879, 104064 |
| 61688 | 50715758 | 50717268 | 138592 |
| 61689 | 50977691 | 50979972 | 092439 |
| 61690 | 50989375 | 50990053 | |
| 61691 | 51010438 | 51011473 | 138600 |
| 61692 | 51056631 | 51058710 | 138600 |
| 61693 | 51059447 | 51060411 | |
| 61694 | 51070165 | 51070788 | |
| 61695 | 51149376 | 51150744 | |
| 61696 | 51165787 | 51166628 | |
| 61697 | 51176576 | 51178682 | |
| 61698 | 51199052 | 51201804 | 081014 |
| 61699 | 51321864 | 51323925 | |
| 61700 | 51494818 | 51498135 | 259240 |
| 61701 | 51580574 | 51581295 | 259240, 137869 |
| 61702 | 51785847 | 51786921 | 104093, 259678 |
| 61703 | 51868670 | 51869619 | 104093 |
| 61704 | 51914090 | 51916081 | 104093 |
| 61705 | 52018475 | 52019074 | 140280 |
| 61706 | 52023359 | 52024338 | 140280 |
| 61707 | 52028818 | 52030579 | 140280 |
| 61708 | 52043831 | 52044399 | 128872 |
| 61709 | 52108692 | 52109270 | |
| 61710 | 52121524 | 52123042 | 138594 |
| 61711 | 52135067 | 52136211 | 138594 |
| 61712 | 52160848 | 52161806 | 138594 |
| 61713 | 52166432 | 52167868 | 138594 |
| 61714 | 52199117 | 52200159 | 272337, 138594 |
| 61715 | 52263114 | 52264880 | 166477, 069956 |
| 61716 | 52273439 | 52275030 | 069956 |
| 61717 | 52310494 | 52314713 | 259438, 069956 |
| 61718 | 52344669 | 52345630 | 069956 |
| 61719 | 52360373 | 52362714 | 259712 |
| 61720 | 52368288 | 52371628 | 259712 |
| 61721 | 52385868 | 52386599 | 259712 |
| 61722 | 52403759 | 52405390 | 137875 |
| 61723 | 52407861 | 52409152 | 259327 |
| 61724 | 52425695 | 52428204 | 259709, 069966 |
| 61725 | 52429059 | 52430393 | 259709, 069966 |
| 61726 | 52448113 | 52449247 | 069966 |
| 61727 | 52470716 | 52472641 | 259577, 069966 |
| 61728 | 52491384 | 52491953 | 128833, 259577 |
| 61729 | 52579877 | 52581559 | 128833 |
| 61730 | 52586999 | 52588280 | 128833 |
| 61731 | 52820797 | 52822294 | 197535 |
| 61732 | 52858938 | 52861687 | 128989 |
| 61733 | 52866637 | 52867364 | |
| 61734 | 52969784 | 52972084 | 047346 |
| 61735 | 53001077 | 53002860 | 047346 |
| 61736 | 53038056 | 53039130 | |
| 61737 | 53072595 | 53073262 | 169856 |
| 61738 | 53075250 | 53079994 | 169856 |
| 61739 | 53080037 | 53083778 | 169856 |
| 61740 | 53089817 | 53091689 | |
| 61741 | 53091758 | 53092988 | |
| 61742 | 53094370 | 53099774 | 259203 |
| 61743 | 53325833 | 53328266 | |
| 61744 | 53351067 | 53352468 | |
| 61745 | 53364167 | 53364847 | |
| 61746 | 53489054 | 53490464 | |
| 61747 | 53738482 | 53742429 | |
| 61748 | 53744464 | 53748696 | |
| 61749 | 53825628 | 53827329 | 166415 |
| 61750 | 53851443 | 53852216 | 166415 |
| 61751 | 53872481 | 53872975 | 166415 |
| 61752 | 53917428 | 53918083 | 166415 |
| 61753 | 53967754 | 53968932 | 166415 |
| 61754 | 54013473 | 54014384 | 166415 |
| 61755 | 54051771 | 54052225 | 166415 |
| 61756 | 54202076 | 54202808 | |
| 61757 | 55487978 | 55490098 | 137876 |
| 61758 | 55513151 | 55513846 | 069974 |
| 61759 | 55541973 | 55543056 | 069974 |
| 61760 | 55548526 | 55549468 | 069974 |
| 61761 | 55571749 | 55572751 | 069974 |
| 61762 | 55580893 | 55583140 | 069974 |
| 61763 | 55610521 | 55612152 | 069943, 225973, 069974 |
| 61764 | 55694273 | 55696868 | 261771, 260916 |
| 61765 | 55699439 | 55701225 | 261652, 260916, 261771 |
| 61766 | 56160801 | 56161958 | 069869 |
| 61767 | 56243855 | 56245422 | 069869 |
| 61768 | 56284979 | 56286888 | 069869 |
| 61769 | 56331385 | 56333936 | |
| 61770 | 56534968 | 56538667 | 151575, 181827 |
| 61771 | 56657170 | 56658071 | 151575 |
| 61772 | 56945884 | 56947214 | 137871 |
| 61773 | 57024430 | 57026500 | 137871 |
| 61774 | 57064989 | 57066460 | 137871 |
| 61775 | 57074350 | 57075183 | 137871 |
| 61776 | 57123832 | 57125141 | 137871 |
| 61777 | 57163302 | 57163874 | 137871 |
| 61778 | 57178457 | 57180560 | 137871 |
| 61779 | 57209696 | 57212738 | 140262, 137871 |
| 61780 | 57257628 | 57258150 | 140262 |
| 61781 | 57307266 | 57309144 | 140262 |
| 61782 | 57410004 | 57410979 | 140262 |
| 61783 | 57598323 | 57599326 | 247982 |
| 61784 | 57668291 | 57670113 | 128849 |
| 61785 | 57670801 | 57672432 | 128849 |
| 61786 | 57672859 | 57673576 | 128849 |
| 61787 | 57697885 | 57699203 | 128849 |
| 61788 | 57710596 | 57712029 | 128849 |
| 61789 | 57718823 | 57720429 | 128849 |
| 61790 | 57729308 | 57730640 | 128849 |
| 61791 | 57732876 | 57733555 | 128849 |
| 61792 | 57768887 | 57771583 | 261489, 128849 |
| 61793 | 57777073 | 57779415 | 128849 |
| 61794 | 57789134 | 57789798 | 128849 |
| 61795 | 57851903 | 57852694 | |
| 61796 | 57911579 | 57912534 | 137878, 255529, 263155 |
| 61797 | 57959207 | 57960099 | 137878, 255529, 263155 |
| 61798 | 57998007 | 58000111 | 137878, 255529 |
| 61799 | 58170503 | 58171374 | |
| 61800 | 58213094 | 58214202 | |
| 61801 | 58214885 | 58216297 | |
| 61802 | 58409738 | 58410439 | 128918 |
| 61803 | 58428318 | 58435468 | 103569, 128918 |
| 61804 | 58437677 | 58441922 | 128918, 103569 |
| 61805 | 58524483 | 58526710 | 128918 |
| 61806 | 58540003 | 58540824 | 128918 |
| 61807 | 58574350 | 58574924 | 128918 |
| 61808 | 58575507 | 58578910 | 128918 |
| 61809 | 58598218 | 58600963 | 128918 |
| 61810 | 58621066 | 58626675 | 128918 |
| 61811 | 58693814 | 58694917 | 128918 |
| 61812 | 58712074 | 58713837 | 128918, 166035 |
| 61813 | 58715083 | 58718427 | 128918, 166035 |
| 61814 | 58722354 | 58726769 | 128918, 166035 |
| 61815 | 58729820 | 58735964 | 128918, 259293, 166035 |
| 61816 | 58736074 | 58738236 | 128918, 259293, 166035 |
| 61817 | 58749874 | 58752345 | 128918, 259293, 166035 |
| 61818 | 58756959 | 58769860 | 128918, 259293, 166035 |
| 61819 | 58816443 | 58817056 | 166035 |
| 61820 | 58819058 | 58822147 | 166035 |
| 61821 | 58837783 | 58840831 | 166035 |
| 61822 | 58865327 | 58865834 | |
| 61823 | 59040475 | 59042537 | 137845 |
| 61824 | 59062232 | 59064667 | 128923, 245975 |
| 61825 | 59224085 | 59226955 | 137776, 157450 |
| 61826 | 59279033 | 59281146 | 157450 |
| 61827 | 59327137 | 59327982 | 157450 |
| 61828 | 59457315 | 59459199 | 157483 |
| 61829 | 59547451 | 59549983 | 157483 |
| 61830 | 59563638 | 59567304 | 157483 |
| 61831 | 59575818 | 59576658 | 157483 |
| 61832 | 59586493 | 59588464 | 157483 |
| 61833 | 59631266 | 59632035 | 157483 |
| 61834 | 59641693 | 59643286 | 157483, 259735 |
| 61835 | 59662877 | 59666160 | 157470, 157483 |
| 61836 | 59702355 | 59703280 | 157470 |
| 61837 | 59903346 | 59904701 | 140297 |
| 61838 | 59948884 | 59949995 | 140307, 227161 |
| 61839 | 59980210 | 59982460 | 259238, 140299 |
| 61840 | 59989805 | 59990712 | |
| 61841 | 60030653 | 60032862 | |
| 61842 | 60298292 | 60299268 | 171956 |
| 61843 | 60383211 | 60384029 | 259223 |
| 61844 | 60385653 | 60386367 | 259223 |
| 61845 | 60419596 | 60420623 | |
| 61846 | 60631997 | 60633300 | |
| 61847 | 60663478 | 60664079 | 182718 |
| 61848 | 60682948 | 60691308 | 182718 |
| 61849 | 60694751 | 60695441 | 182718 |
| 61850 | 60699003 | 60701725 | |
| 61851 | 60761998 | 60762616 | 128915 |
| 61852 | 60769746 | 60772283 | 245534, 128915 |
| 61853 | 60773000 | 60773392 | 245534 |
| 61854 | 60848031 | 60848478 | 245534, 069667 |
| 61855 | 60860352 | 60861196 | 245534, 069667 |
| 61856 | 60867480 | 60868632 | 245534, 069667 |
| 61857 | 60874451 | 60875090 | 245534, 069667 |
| 61858 | 60882631 | 60885742 | 245534, 069667 |
| 61859 | 60886006 | 60887082 | 245534, 069667 |
| 61860 | 60973376 | 60976161 | 259482, 069667 |
| 61861 | 61027061 | 61028385 | 069667 |
| 61862 | 61032034 | 61033866 | 069667 |
| 61863 | 61043877 | 61046073 | 069667 |
| 61864 | 61090837 | 61091918 | 069667 |
| 61865 | 61191377 | 61192621 | 069667 |
| 61866 | 61205090 | 61206942 | 069667 |
| 61867 | 61208184 | 61210423 | 069667 |
| 61868 | 61341903 | 61343340 | 069667 |
| 61869 | 61361546 | 61363333 | 069667 |
| 61870 | 61387724 | 61388718 | 069667 |
| 61871 | 61389166 | 61389763 | 069667 |
| 61872 | 61519052 | 61522427 | 069667 |
| 61873 | 61618522 | 61619937 | 259616 |
| 61874 | 61666648 | 61667562 | 259616 |
| 61875 | 61674990 | 61675963 | 259616 |
| 61876 | 61686115 | 61686801 | 259616 |
| 61877 | 61694345 | 61695078 | 259616 |
| 61878 | 61695648 | 61697345 | 259616 |
| 61879 | 61703990 | 61704757 | 259616 |
| 61880 | 61713585 | 61715335 | 259616 |
| 61881 | 61771716 | 61772376 | 259616 |
| 61882 | 62199146 | 62200615 | 129003 |
| 61883 | 62351676 | 62353252 | 259251, 129003 |
| 61884 | 62401618 | 62406555 | |
| 61885 | 62407766 | 62408789 | |
| 61886 | 62410596 | 62412930 | |
| 61887 | 62415652 | 62417627 | |
| 61888 | 62427408 | 62428510 | |
| 61889 | 62580397 | 62581275 | |
| 61890 | 62594375 | 62595135 | |
| 61891 | 62681866 | 62684883 | 171914 |
| 61892 | 62797655 | 62799388 | 171914 |
| 61893 | 62862141 | 62862953 | 171914 |
| 61894 | 62895752 | 62896330 | 171914 |
| 61895 | 63049383 | 63051693 | 171914 |
| 61896 | 63074060 | 63075195 | 171914 |
| 61897 | 63104308 | 63105082 | 171914 |
| 61898 | 63127071 | 63128735 | 259370, 171914 |
| 61899 | 63133986 | 63134543 | 259370, 171914 |
| 61900 | 63142063 | 63143822 | 259370 |
| 61901 | 63144327 | 63146904 | 259370 |
| 61902 | 63177472 | 63178278 | |
| 61903 | 63178305 | 63183289 | |
| 61904 | 63186340 | 63193103 | 259727 |
| 61905 | 63227128 | 63228691 | |
| 61906 | 63248634 | 63249903 | |
| 61907 | 63249970 | 63251343 | |
| 61908 | 63264584 | 63265883 | |
| 61909 | 63268125 | 63268990 | |
| 61910 | 63279082 | 63279690 | |
| 61911 | 63287027 | 63287563 | |
| 61912 | 63302482 | 63304795 | |
| 61913 | 63306521 | 63309272 | |
| 61914 | 63333077 | 63344600 | 259498, 140416 |
| 61915 | 63344836 | 63347810 | 140416 |
| 61916 | 63399959 | 63402437 | 259672 |
| 61917 | 63413242 | 63414990 | 103642 |
| 61918 | 63447920 | 63450725 | 185088 |
| 61919 | 63481100 | 63482638 | 166128 |
| 61920 | 63490767 | 63491369 | 166128 |
| 61921 | 63503954 | 63505920 | 166128 |
| 61922 | 63509573 | 63512023 | 166128 |
| 61923 | 63513379 | 63514624 | 166128 |
| 61924 | 63521204 | 63522459 | 166128 |
| 61925 | 63539165 | 63539774 | 166128 |
| 61926 | 63568676 | 63570457 | 138613 |
| 61927 | 63640561 | 63641161 | 074410 |
| 61928 | 63663809 | 63664994 | 074410 |
| 61929 | 63673539 | 63674477 | 074410 |
| 61930 | 63736733 | 63737340 | |
| 61931 | 63741522 | 63743083 | |
| 61932 | 63765064 | 63769482 | |
| 61933 | 63789814 | 63790668 | |
| 61934 | 63792436 | 63798148 | 140455 |
| 61935 | 63801650 | 63803562 | 140455 |
| 61936 | 63806298 | 63807358 | 140455 |
| 61937 | 63815295 | 63816019 | 140455 |
| 61938 | 63891567 | 63894565 | 259248, 197361 |
| 61939 | 64007943 | 64008450 | 103657 |
| 61940 | 64009856 | 64012689 | 103657 |
| 61941 | 64020340 | 64021247 | 103657 |
| 61942 | 64125178 | 64127035 | 103657 |
| 61943 | 64174749 | 64175474 | |
| 61944 | 64180234 | 64180980 | |
| 61945 | 64188300 | 64189767 | |
| 61946 | 64259842 | 64260785 | 035664 |
| 61947 | 64261647 | 64267005 | 035664 |
| 61948 | 64296699 | 64297613 | 035664 |
| 61949 | 64307819 | 64312441 | 035664 |
| 61950 | 64319613 | 64320303 | 035664 |
| 61951 | 64337777 | 64339301 | 035664 |
| 61952 | 64342564 | 64343211 | 035664 |
| 61953 | 64362346 | 64365964 | 166797, 035664 |
| 61954 | 64384404 | 64386943 | 028528, 166797 |
| 61955 | 64387923 | 64389759 | 028528 |
| 61956 | 64397305 | 64397832 | 028528 |
| 61957 | 64403676 | 64405980 | 201071, 028528 |
| 61958 | 64454037 | 64456525 | 166794 |
| 61959 | 64509064 | 64509738 | 169118, 259316 |
| 61960 | 64539638 | 64540770 | 169118, 259316 |
| 61961 | 64541679 | 64542095 | 169118, 259316 |
| 61962 | 64647545 | 64649135 | 169118, 259316 |
| 61963 | 64679335 | 64680769 | 103671, 166803 |
| 61964 | 64735438 | 64737342 | 206781, 103671 |
| 61965 | 64752348 | 64754616 | 180357 |
| 61966 | 64828377 | 64828809 | 180357 |
| 61967 | 64943599 | 64944276 | 180357 |
| 61968 | 64985130 | 64986016 | 180304 |
| 61969 | 64994554 | 64996358 | 180304, 259635 |
| 61970 | 64996572 | 64997854 | 259635 |
| 61971 | 65023081 | 65025320 | |
| 61972 | 65066839 | 65068687 | 166831 |
| 61973 | 65101969 | 65102682 | |
| 61974 | 65133463 | 65134552 | 249240, 241839 |
| 61975 | 65230289 | 65230877 | 166839 |
| 61976 | 65273995 | 65274640 | 090487 |
| 61977 | 65281104 | 65282638 | 090487 |
| 61978 | 65321113 | 65322312 | 103707 |
| 61979 | 65325116 | 65326707 | |
| 61980 | 65336455 | 65338373 | 186198 |
| 61981 | 65346557 | 65348744 | 103710 |
| 61982 | 65348775 | 65350339 | 103710 |
| 61983 | 65376362 | 65377636 | |
| 61984 | 65378623 | 65382371 | |
| 61985 | 65425073 | 65426587 | 090470 |
| 61986 | 65465979 | 65466529 | 166855 |
| 61987 | 65475840 | 65478650 | 166855 |
| 61988 | 65567288 | 65568958 | 138617 |
| 61989 | 65578006 | 65579510 | 138617 |
| 61990 | 65587600 | 65589095 | 199568, 138617 |
| 61991 | 65595564 | 65597988 | 200156 |
| 61992 | 65604602 | 65605335 | |
| 61993 | 65677711 | 65678526 | 103742 |
| 61994 | 65808867 | 65810295 | 074603 |
| 61995 | 65822342 | 65823733 | 074696 |
| 61996 | 65824319 | 65825625 | 074696 |
| 61997 | 65902636 | 65904915 | 074621, 138614 |
| 61998 | 65947306 | 65947988 | 074621 |
| 61999 | 65958528 | 65959484 | 174485 |
| 62000 | 66079875 | 66080501 | 103769, 174485 |
| 62001 | 66082818 | 66085519 | 174485, 103769 |
| 62002 | 66088823 | 66089393 | 103769 |
| 62003 | 66093533 | 66094193 | 103769 |
| 62004 | 66124656 | 66126428 | 103769 |
| 62005 | 66161139 | 66163897 | 103769 |
| 62006 | 66585085 | 66588031 | 166938, 261318 |
| 62007 | 66648230 | 66649384 | 075131 |
| 62008 | 66662072 | 66663127 | 075131 |
| 62009 | 66678320 | 66680569 | 169032, 075131 |
| 62010 | 66746445 | 66746846 | 169032 |
| 62011 | 66764562 | 66765839 | 169032 |
| 62012 | 66773030 | 66776287 | 169032 |
| 62013 | 66781290 | 66782107 | 261351, 269999, 169032 |
| 62014 | 66789139 | 66790900 | 266589, 174444, 174446 |
| 62015 | 66795492 | 66798266 | 200623, 174442, 174444 |
| 62016 | 66882728 | 66883336 | 259471 |
| 62017 | 66903416 | 66904106 | 259471 |
| 62018 | 66909280 | 66910680 | 259471 |
| 62019 | 66942102 | 66943369 | 259471 |
| 62020 | 66944437 | 66945059 | 259471 |
| 62021 | 66992796 | 66996242 | 137834 |
| 62022 | 67041721 | 67045049 | 137834 |
| 62023 | 67057947 | 67059131 | 137834 |
| 62024 | 67071534 | 67072634 | 137834 |
| 62025 | 67095006 | 67095976 | |
| 62026 | 67133004 | 67136098 | |
| 62027 | 67143223 | 67144078 | |
| 62028 | 67175476 | 67176539 | |
| 62029 | 67219631 | 67220046 | 259437 |
| 62030 | 67223212 | 67225008 | 259289, 259437 |
| 62031 | 67237840 | 67239579 | 259289 |
| 62032 | 67286158 | 67286912 | 259347 |
| 62033 | 67315984 | 67318718 | 259347 |
| 62034 | 67336440 | 67337233 | 259347 |
| 62035 | 67339812 | 67342918 | 259347 |
| 62036 | 67349565 | 67352463 | 259347 |
| 62037 | 67354329 | 67361477 | 166949 |
| 62038 | 67374251 | 67374979 | 166949 |
| 62039 | 67396664 | 67403001 | 166949 |
| 62040 | 67406975 | 67411037 | 166949 |
| 62041 | 67417965 | 67421285 | 166949 |
| 62042 | 67426458 | 67428689 | 166949 |
| 62043 | 67438484 | 67443295 | 259202, 166949 |
| 62044 | 67460042 | 67460973 | 166949 |
| 62045 | 67466489 | 67468574 | 166949 |
| 62046 | 67545798 | 67547930 | 103599, 103591 |
| 62047 | 67650817 | 67652270 | 103599 |
| 62048 | 67812575 | 67814973 | 189227, 259673 |
| 62049 | 67833871 | 67836251 | 137764, 270964 |
| 62050 | 67842537 | 67843332 | 137764 |
| 62051 | 68131180 | 68131750 | 245719, 259410 |
| 62052 | 68131833 | 68133323 | 206625, 259410 |
| 62053 | 68176533 | 68177951 | |
| 62054 | 68207219 | 68208629 | |
| 62055 | 68334435 | 68335347 | |
| 62056 | 68346281 | 68348284 | 033800 |
| 62057 | 68398718 | 68399399 | 033800 |
| 62058 | 68497362 | 68498103 | 260007, 129007 |
| 62059 | 68521041 | 68522940 | 264779, 260007, 128973 |
| 62060 | 68548560 | 68550304 | 128973 |
| 62061 | 68563349 | 68571913 | 169018, 260657 |
| 62062 | 68577557 | 68578098 | 169018 |
| 62063 | 68592503 | 68593401 | 261425 |
| 62064 | 68601322 | 68602086 | 137809 |
| 62065 | 68692341 | 68692848 | 137809 |
| 62066 | 68834706 | 68836391 | |
| 62067 | 69085288 | 69086171 | 140350 |
| 62068 | 69108319 | 69114232 | 258484, 140350 |
| 62069 | 69366332 | 69367549 | 212766 |
| 62070 | 69418122 | 69419159 | |
| 62071 | 69420745 | 69421855 | |
| 62072 | 69452327 | 69454354 | 266374, 138604 |
| 62073 | 69498249 | 69498974 | 138604 |
| 62074 | 69516625 | 69517326 | 138604 |
| 62075 | 69597588 | 69598394 | 137819 |
| 62076 | 69706680 | 69707799 | 259426, 137807 |
| 62077 | 69743710 | 69746346 | 137818 |
| 62078 | 69754515 | 69756080 | 245750 |
| 62079 | 69758258 | 69758758 | 245750 |
| 62080 | 69845749 | 69847739 | 245750 |
| 62081 | 69848454 | 69851400 | 245750 |
| 62082 | 69853006 | 69855482 | 245750 |
| 62083 | 69871099 | 69873677 | |
| 62084 | 69884061 | 69884819 | 259641 |
| 62085 | 69886572 | 69887665 | 259641 |
| 62086 | 69920010 | 69920713 | 259641 |
| 62087 | 70357266 | 70357937 | 140332 |
| 62088 | 70371753 | 70372643 | 207965, 140332 |
| 62089 | 70387768 | 70394285 | 140332 |
| 62090 | 70413905 | 70415258 | |
| 62091 | 70419354 | 70420794 | |
| 62092 | 70432394 | 70433900 | |
| 62093 | 70451500 | 70453758 | |
| 62094 | 70457164 | 70458695 | |
| 62095 | 70487469 | 70490698 | 259252 |
| 62096 | 70563018 | 70564224 | |
| 62097 | 70577150 | 70577590 | |
| 62098 | 70641117 | 70641872 | |
| 62099 | 70669160 | 70670019 | |
| 62100 | 70725350 | 70727532 | |
| 62101 | 70752263 | 70753071 | |
| 62102 | 70766609 | 70768289 | |
| 62103 | 70800147 | 70800726 | |
| 62104 | 70877024 | 70878366 | |
| 62105 | 70909306 | 70910287 | |
| 62106 | 71006315 | 71007701 | 137831 |
| 62107 | 71007813 | 71009207 | 137831 |
| 62108 | 71033285 | 71034593 | 137831 |
| 62109 | 71054276 | 71056705 | 137831 |
| 62110 | 71058083 | 71058793 | |
| 62111 | 71063010 | 71063566 | 259624 |
| 62112 | 71066907 | 71068807 | 259624 |
| 62113 | 71079329 | 71082164 | |
| 62114 | 71085783 | 71088200 | |
| 62115 | 71092252 | 71092804 | 235420 |
| 62116 | 71099145 | 71100431 | |
| 62117 | 71118376 | 71119800 | |
| 62118 | 71122885 | 71123734 | 267539 |
| 62119 | 71127632 | 71128308 | 166173 |
| 62120 | 71145301 | 71147342 | 137821, 166173 |
| 62121 | 71148223 | 71150173 | 137821 |
| 62122 | 71167143 | 71168376 | 137821 |
| 62123 | 71183899 | 71185507 | 129028, 137821 |
| 62124 | 71371670 | 71372287 | |
| 62125 | 71406462 | 71409503 | 225362, 187720 |
| 62126 | 71428658 | 71429826 | 187720 |
| 62127 | 71435596 | 71436761 | 187720 |
| 62128 | 71437756 | 71440852 | 259964, 187720 |
| 62129 | 71441772 | 71442873 | 259964, 187720 |
| 62130 | 71447023 | 71447596 | 259964, 187720 |
| 62131 | 71529018 | 71533159 | 187720 |
| 62132 | 71543925 | 71545175 | 187720 |
| 62133 | 71570359 | 71573743 | 187720 |
| 62134 | 71585794 | 71589374 | 187720 |
| 62135 | 71612102 | 71612832 | 187720 |
| 62136 | 71620037 | 71622569 | 187720 |
| 62137 | 71640746 | 71641690 | 260586, 187720 |
| 62138 | 71658751 | 71659707 | 187720 |
| 62139 | 71672108 | 71673279 | 187720 |
| 62140 | 71736317 | 71738217 | 187720 |
| 62141 | 71739681 | 71741664 | 187720 |
| 62142 | 71793688 | 71794099 | 187720 |
| 62143 | 71901461 | 71903097 | 187720 |
| 62144 | 71946230 | 71947628 | 187720 |
| 62145 | 71948627 | 71950282 | 187720 |
| 62146 | 71951067 | 71953907 | 187720 |
| 62147 | 71969057 | 71970677 | 187720 |
| 62148 | 71983952 | 71984584 | 187720 |
| 62149 | 71999760 | 72000371 | 187720 |
| 62150 | 72024867 | 72025567 | 187720 |
| 62151 | 72038938 | 72041933 | 187720 |
| 62152 | 72044056 | 72046624 | 187720 |
| 62153 | 72053032 | 72055022 | 187720 |
| 62154 | 72084878 | 72085242 | 031544 |
| 62155 | 72402652 | 72403241 | 066933 |
| 62156 | 72409519 | 72411433 | 066933, 166192 |
| 62157 | 72502976 | 72503534 | 067225 |
| 62158 | 72522017 | 72523099 | 067225 |
| 62159 | 72523154 | 72524522 | 067225 |
| 62160 | 72564257 | 72565487 | 137817, 273025 |
| 62161 | 72667480 | 72669455 | 260339, 260729, 213614 |
| 62162 | 72754089 | 72755041 | 261423 |
| 62163 | 72765314 | 72768111 | 166233, 261423 |
| 62164 | 72963053 | 72963746 | |
| 62165 | 72977618 | 72979663 | 140463, 175202 |
| 62166 | 73074808 | 73077431 | 260898, 159322 |
| 62167 | 73089074 | 73089916 | 260898 |
| 62168 | 73250868 | 73251417 | |
| 62169 | 73343369 | 73345961 | 067141 |
| 62170 | 73457568 | 73459196 | 067141 |
| 62171 | 73585856 | 73586778 | 067141 |
| 62172 | 73680016 | 73681249 | |
| 62173 | 73832436 | 73832923 | 183324 |
| 62174 | 73892273 | 73894387 | 156642 |
| 62175 | 73921082 | 73922936 | 156642 |
| 62176 | 73924650 | 73927445 | 156642 |
| 62177 | 73928752 | 73929474 | |
| 62178 | 73975781 | 73979406 | 103855 |
| 62179 | 74273955 | 74274643 | |
| 62180 | 74279657 | 74282227 | 067221 |
| 62181 | 74283461 | 74284906 | 067221 |
| 62182 | 74286659 | 74288142 | 140464, 067221 |
| 62183 | 74302665 | 74304547 | 140464 |
| 62184 | 74492563 | 74493833 | 137868 |
| 62185 | 74532288 | 74532813 | 140481 |
| 62186 | 74657650 | 74658790 | 261821, 140459 |
| 62187 | 74687340 | 74690730 | |
| 62188 | 74710627 | 74711833 | 138623 |
| 62189 | 74725292 | 74726314 | 138623 |
| 62190 | 74735342 | 74736402 | |
| 62191 | 74736952 | 74737667 | |
| 62192 | 74752627 | 74754059 | 247240, 138629 |
| 62193 | 74817356 | 74818766 | |
| 62194 | 74832536 | 74834412 | 179361 |
| 62195 | 74838260 | 74838985 | 179361 |
| 62196 | 74890421 | 74891600 | 179335, 179361 |
| 62197 | 74906978 | 74909226 | 260919, 179335 |
| 62198 | 74943491 | 74944062 | 179151 |
| 62199 | 74956074 | 74957324 | 179151 |
| 62200 | 74987446 | 74988765 | 179151 |
| 62201 | 75017506 | 75019817 | 140465 |
| 62202 | 75022723 | 75024543 | |
| 62203 | 75042007 | 75042715 | 140505 |
| 62204 | 75044725 | 75045362 | 140505 |
| 62205 | 75072068 | 75072745 | |
| 62206 | 75073152 | 75075506 | 103653 |
| 62207 | 75091825 | 75093101 | 103653 |
| 62208 | 75108944 | 75110083 | 261606, 140506 |
| 62209 | 75135122 | 75136429 | 140497, 140474 |
| 62210 | 75165111 | 75166432 | 140497 |
| 62211 | 75182112 | 75183260 | 178802 |
| 62212 | 75183345 | 75184310 | 178802 |
| 62213 | 75198125 | 75199940 | 178761 |
| 62214 | 75229258 | 75231115 | 178741 |
| 62215 | 75248503 | 75249812 | 198794, 178718 |
| 62216 | 75250836 | 75251448 | 198794 |
| 62217 | 75276089 | 75276653 | 198794 |
| 62218 | 75287544 | 75288735 | 198794 |
| 62219 | 75315379 | 75316898 | 138621, 260483 |
| 62220 | 75321501 | 75322653 | 138621 |
| 62221 | 75493749 | 75496088 | 167173 |
| 62222 | 75497028 | 75497596 | 167173 |
| 62223 | 75628088 | 75629138 | 140365 |
| 62224 | 75639270 | 75641063 | 140398 |
| 62225 | 75660010 | 75662029 | 260274, 169375, 140400 |
| 62226 | 75742799 | 75748912 | 169375 |
| 62227 | 75752721 | 75753622 | |
| 62228 | 75824721 | 75825265 | 169410, 260269 |
| 62229 | 75869799 | 75872471 | 169410, 260269 |
| 62230 | 75917266 | 75919123 | 169371, 260235 |
| 62231 | 75931198 | 75933416 | 177971, 260206 |
| 62232 | 75939025 | 75941305 | 173548, 177971 |
| 62233 | 75943501 | 75944263 | 173548 |
| 62234 | 75953415 | 75954215 | 173548 |
| 62235 | 75956043 | 75956925 | |
| 62236 | 76010723 | 76011080 | |
| 62237 | 76013104 | 76013748 | |
| 62238 | 76016050 | 76016783 | 182950 |
| 62239 | 76050544 | 76051619 | 260288 |
| 62240 | 76067578 | 76069670 | 187812 |
| 62241 | 76134936 | 76136814 | 140367 |
| 62242 | 76195467 | 76197200 | 167196 |
| 62243 | 76601918 | 76606725 | 140374 |
| 62244 | 76646198 | 76646567 | 140386 |
| 62245 | 76869040 | 76869584 | 140386 |
| 62246 | 77196580 | 77198412 | 140386 |
| 62247 | 77223531 | 77224796 | 117906 |
| 62248 | 77355400 | 77358169 | 140391 |
| 62249 | 77361778 | 77363662 | 140391 |
| 62250 | 77710653 | 77714618 | 140382, 173517 |
| 62251 | 77715931 | 77718537 | 140382 |
| 62252 | 77756634 | 77757380 | 140382 |
| 62253 | 77789027 | 77789681 | |
| 62254 | 77861159 | 77862474 | 259420 |
| 62255 | 78236284 | 78237214 | 214646 |
| 62256 | 78283823 | 78285003 | 261143, 167202 |
| 62257 | 78295822 | 78298145 | 167202 |
| 62258 | 78298970 | 78301274 | 167202 |
| 62259 | 78302337 | 78302935 | 167202 |
| 62260 | 78304277 | 78305753 | 167202 |
| 62261 | 78309704 | 78312050 | 167202 |
| 62262 | 78348294 | 78350268 | 167202 |
| 62263 | 78356485 | 78358873 | 167202 |
| 62264 | 78367617 | 78370330 | 183476, 167202 |
| 62265 | 78423494 | 78424658 | 166411, 136425 |
| 62266 | 78440851 | 78442680 | 166411 |
| 62267 | 78508897 | 78509627 | 103740 |
| 62268 | 78584026 | 78584916 | 140395 |
| 62269 | 78591013 | 78592652 | 140395 |
| 62270 | 78666508 | 78667918 | |
| 62271 | 78689202 | 78689876 | |
| 62272 | 78729428 | 78731805 | 136381 |
| 62273 | 78799454 | 78800697 | 188266 |
| 62274 | 78832033 | 78834167 | 041357 |
| 62275 | 78943240 | 78943985 | 117971 |
| 62276 | 79151899 | 79153294 | 185787 |
| 62277 | 79164509 | 79167024 | 185787 |
| 62278 | 79197337 | 79199882 | |
| 62279 | 79223097 | 79224514 | 103811 |
| 62280 | 79236528 | 79237679 | 103811 |
| 62281 | 79242284 | 79245077 | |
| 62282 | 79262216 | 79263142 | 058335 |
| 62283 | 79927042 | 79930021 | |
| 62284 | 79931376 | 79934064 | |
| 62285 | 79937816 | 79938768 | |
| 62286 | 79947200 | 79949261 | |
| 62287 | 79949964 | 79951246 | |
| 62288 | 80061251 | 80061952 | |
| 62289 | 80138632 | 80139774 | 136371, 259332 |
| 62290 | 80145745 | 80146426 | 136371, 259332 |
| 62291 | 80164373 | 80167121 | 136371, 259332 |
| 62292 | 80174003 | 80174508 | 136371, 259332 |
| 62293 | 80178071 | 80179134 | 136371, 259332 |
| 62294 | 80187586 | 80191703 | 180953, 136371, 259332 |
| 62295 | 80214689 | 80217083 | 259642, 257028, 180953, |
| | | | 259332 |
| 62296 | 80248433 | 80249340 | |
| 62297 | 80252641 | 80253758 | 140379 |
| 62298 | 80257866 | 80259348 | 140379 |
| 62299 | 80265172 | 80265704 | |
| 62300 | 80351261 | 80353234 | 086666 |
| 62301 | 80364430 | 80365127 | 086666 |
| 62302 | 80444147 | 80447236 | 103876 |
| 62303 | 80451905 | 80456170 | 103876 |
| 62304 | 80463852 | 80465242 | 103876 |
| 62305 | 80465501 | 80467739 | 103876 |
| 62306 | 80471358 | 80472622 | 103876 |
| 62307 | 80487241 | 80487759 | |
| 62308 | 80499777 | 80500933 | 259417 |
| 62309 | 80501616 | 80503837 | 259417 |
| 62310 | 80521458 | 80522100 | 259417 |
| 62311 | 80543220 | 80545070 | 259417 |
| 62312 | 80552137 | 80553297 | |
| 62313 | 80711400 | 80712451 | 172379 |
| 62314 | 80966354 | 80967298 | |
| 62315 | 80986712 | 80988274 | 136379 |
| 62316 | 80988353 | 80989163 | 136379 |
| 62317 | 81068653 | 81069362 | |
| 62318 | 81142649 | 81144579 | 103888 |
| 62319 | 81149248 | 81149819 | 103888 |
| 62320 | 81281123 | 81282998 | 117899 |
| 62321 | 81292023 | 81295115 | 140406 |
| 62322 | 81330774 | 81332253 | 156206 |
| 62323 | 81405400 | 81406015 | 156206 |
| 62324 | 81565873 | 81566698 | 172349 |
| 62325 | 81567074 | 81568786 | 172349 |
| 62326 | 81677537 | 81678355 | |
| 62327 | 81691056 | 81691992 | |
| 62328 | 82337729 | 82339874 | 183496 |
| 62329 | 82415105 | 82415480 | |
| 62330 | 82419855 | 82421576 | |
| 62331 | 82438521 | 82439818 | 140598 |
| 62332 | 82555669 | 82556929 | 188659 |
| 62333 | 83378025 | 83378673 | 103723 |
| 62334 | 83418106 | 83420137 | 250988, 259442 |
| 62335 | 83477966 | 83480091 | 156232 |
| 62336 | 83496753 | 83497244 | 156232 |
| 62337 | 83501201 | 83502564 | 156232 |
| 62338 | 83525172 | 83525861 | 103942 |
| 62339 | 83587248 | 83588725 | 103942 |
| 62340 | 83604549 | 83606998 | 259805, 103942 |
| 62341 | 83610402 | 83612530 | 259805, 103942 |
| 62342 | 83620092 | 83622408 | 259805, 103942 |
| 62343 | 83627109 | 83627899 | 259805, 103942 |
| 62344 | 83653923 | 83655945 | 169612, 103942, 259805 |
| 62345 | 83679554 | 83681262 | 169609, 259805 |
| 62346 | 83734838 | 83736624 | 263643, 064726, 260351, |
| | | | 259805 |
| 62347 | 84321835 | 84324400 | 156218 |
| 62348 | 84325432 | 84326930 | 156218 |
| 62349 | 84366362 | 84368034 | 156218 |
| 62350 | 84401215 | 84403155 | 156218 |
| 62351 | 84513060 | 84514467 | 156218 |
| 62352 | 84516840 | 84517549 | 156218 |
| 62353 | 84599352 | 84600046 | 156218 |
| 62354 | 85112998 | 85114668 | 259728, 189136 |
| 62355 | 85143082 | 85145163 | 176371 |
| 62356 | 85149329 | 85151087 | 176371 |
| 62357 | 85174306 | 85175749 | 176700, 254414 |
| 62358 | 85176469 | 85177677 | 254779, 254414, 176700 |
| 62359 | 85194440 | 85195022 | 177082 |
| 62360 | 85196668 | 85198553 | 197696, 177082 |
| 62361 | 85200832 | 85202223 | 197696 |
| 62362 | 85214757 | 85215553 | 140612 |
| 62363 | 85228268 | 85229080 | 140612 |
| 62364 | 85258449 | 85260749 | 140612 |
| 62365 | 85290468 | 85293292 | 166716 |
| 62366 | 85302020 | 85302714 | 166716 |
| 62367 | 85305900 | 85307214 | 166716 |
| 62368 | 85342659 | 85344270 | 166716 |
| 62369 | 85359767 | 85363136 | 136383 |
| 62370 | 85367321 | 85368045 | 136383 |
| 62371 | 85377868 | 85379055 | 136383 |
| 62372 | 85382073 | 85386233 | 136383 |
| 62373 | 85388141 | 85389381 | 136383 |
| 62374 | 85426561 | 85428709 | 156222 |
| 62375 | 85429052 | 85430479 | 156222 |
| 62376 | 85453725 | 85457255 | 156222 |
| 62377 | 85457394 | 85459013 | 156222 |
| 62378 | 85466973 | 85468307 | 156222 |
| 62379 | 85470871 | 85471826 | 156222 |
| 62380 | 85472931 | 85473824 | 156222 |
| 62381 | 85523347 | 85526929 | 073417 |
| 62382 | 85534983 | 85536696 | 073417 |
| 62383 | 85550999 | 85551627 | 073417 |
| 62384 | 85553771 | 85554838 | 073417 |
| 62385 | 85567740 | 85568932 | 073417 |
| 62386 | 85839760 | 85840539 | 218052 |
| 62387 | 85873882 | 85875464 | |
| 62388 | 85878547 | 85879230 | |
| 62389 | 85879801 | 85880933 | |
| 62390 | 85896841 | 85898412 | |
| 62391 | 85900566 | 85901759 | |
| 62392 | 85922687 | 85924971 | 170776 |
| 62393 | 85950585 | 85951745 | 170776 |
| 62394 | 85955343 | 85958009 | 170776 |
| 62395 | 85986951 | 85987640 | 170776 |
| 62396 | 86046594 | 86047425 | 170776 |
| 62397 | 86106012 | 86107534 | 170776 |
| 62398 | 86122253 | 86127725 | 259375, 170776 |
| 62399 | 86131205 | 86134290 | 170776 |
| 62400 | 86150097 | 86151482 | 170776 |
| 62401 | 86192551 | 86193162 | 259367, 170776 |
| 62402 | 86211023 | 86211713 | 259367, 170776 |
| 62403 | 86215732 | 86216896 | 170776 |
| 62404 | 86250137 | 86253028 | 170776 |
| 62405 | 86265438 | 86266090 | 170776 |
| 62406 | 86295811 | 86296279 | 259762 |
| 62407 | 86305531 | 86306398 | 183655 |
| 62408 | 86313268 | 86316393 | 259195, 221634, 183655 |
| 62409 | 86323801 | 86324830 | 183655 |
| 62410 | 86328246 | 86330413 | 183655 |
| 62411 | 86336620 | 86338740 | 183655 |
| 62412 | 86343389 | 86344074 | |
| 62413 | 86380020 | 86381037 | |
| 62414 | 86383219 | 86384387 | |
| 62415 | 86515861 | 86517180 | |
| 62416 | 86553531 | 86554200 | |
| 62417 | 87315645 | 87316324 | 166748 |
| 62418 | 89008646 | 89011907 | 181991, 259494, 173867 |
| 62419 | 89089122 | 89090462 | 140543 |
| 62420 | 89158135 | 89159487 | |
| 62421 | 89164141 | 89168101 | 181026 |
| 62422 | 89180942 | 89183712 | 172183 |
| 62423 | 89191462 | 89192859 | 172183 |
| 62424 | 89447797 | 89448460 | 140545 |
| 62425 | 89600573 | 89601274 | 260123 |
| 62426 | 89630306 | 89637803 | 140526, 261407 |
| 62427 | 89643205 | 89650682 | 140526 |
| 62428 | 89654309 | 89668137 | 239151, 140526 |
| 62429 | 89668234 | 89673911 | 140526 |
| 62430 | 89675568 | 89677246 | 140526 |
| 62431 | 89679250 | 89684270 | 259699, 140526 |
| 62432 | 89684856 | 89685831 | 140526 |
| 62433 | 89708722 | 89712680 | 140526 |
| 62434 | 89786012 | 89787621 | 140525 |
| 62435 | 89864622 | 89865218 | 140521 |
| 62436 | 89877003 | 89879177 | 261441, 140521 |
| 62437 | 89899577 | 89900341 | |
| 62438 | 89951743 | 89952733 | |
| 62439 | 89979763 | 89980328 | |
| 62440 | 90002251 | 90003754 | 140519 |
| 62441 | 90005437 | 90006750 | 140519 |
| 62442 | 90041471 | 90042633 | |
| 62443 | 90217631 | 90218514 | 166819 |
| 62444 | 90223471 | 90224359 | 166821 |
| 62445 | 90233301 | 90234133 | 140527, 166821 |
| 62446 | 90293249 | 90294615 | 166823 |
| 62447 | 90324352 | 90326015 | |
| 62448 | 90328093 | 90329358 | 166825 |
| 62449 | 90337566 | 90339153 | 166825 |
| 62450 | 90346980 | 90347817 | 166825 |
| 62451 | 90348324 | 90350600 | 166825 |
| 62452 | 90354988 | 90356291 | 166825 |
| 62453 | 90357040 | 90358662 | 166825 |
| 62454 | 90371161 | 90372219 | |
| 62455 | 90388057 | 90389789 | 157823, 250021 |
| 62456 | 90400069 | 90402498 | 157823, 250021 |
| 62457 | 90436151 | 90438067 | 157823, 250021 |
| 62458 | 90455451 | 90456533 | 242498, 250021 |
| 62459 | 90541257 | 90542040 | |
| 62460 | 90544056 | 90546227 | 140548 |
| 62461 | 90546231 | 90548912 | 140548 |
| 62462 | 90561645 | 90562422 | 140548 |
| 62463 | 90571771 | 90572961 | 140548 |
| 62464 | 90578256 | 90579535 | 140548 |
| 62465 | 90579942 | 90582422 | 140548 |
| 62466 | 90618700 | 90619586 | 140548, 259291 |
| 62467 | 90633075 | 90634908 | 182054 |
| 62468 | 90640891 | 90641674 | 182054 |
| 62469 | 90643688 | 90646241 | 259685, 182054 |
| 62470 | 90649641 | 90650510 | 259685 |
| 62471 | 90669350 | 90670427 | 259685 |
| 62472 | 90686281 | 90687111 | |
| 62473 | 90692278 | 90693505 | |
| 62474 | 90696744 | 90699321 | |
| 62475 | 90700676 | 90702893 | |
| 62476 | 90708198 | 90710080 | 240470, 185033 |
| 62477 | 90717471 | 90718787 | 185033 |
| 62478 | 90727079 | 90728964 | 185033 |
| 62479 | 90734989 | 90735855 | 185033 |
| 62480 | 90744253 | 90745546 | 185033 |
| 62481 | 90773288 | 90773858 | 185043 |
| 62482 | 90775756 | 90778195 | 183208, 185043 |
| 62483 | 90805091 | 90805583 | 213471, 261147 |
| 62484 | 90807437 | 90809826 | 182768, 213471, 261147 |
| 62485 | 90863162 | 90863778 | |
| 62486 | 90886365 | 90887060 | |
| 62487 | 90895152 | 90896330 | 196391 |
| 62488 | 90930937 | 90932449 | 140575 |
| 62489 | 90945373 | 90945949 | 140575 |
| 62490 | 91072488 | 91074382 | 140577 |
| 62491 | 91100670 | 91102071 | 140577 |
| 62492 | 91103256 | 91104062 | 140577 |
| 62493 | 91128951 | 91129697 | 140577 |
| 62494 | 91135396 | 91136114 | 140577 |
| 62495 | 91137413 | 91138249 | 140577 |
| 62496 | 91208046 | 91209617 | 245479, 259736 |
| 62497 | 91259984 | 91260630 | 197299, 259736 |
| 62498 | 91402333 | 91405045 | |
| 62499 | 91405910 | 91407974 | |
| 62500 | 91412881 | 91417992 | 140564 |
| 62501 | 91445015 | 91447137 | 196547 |
| 62502 | 91472454 | 91474714 | 140553, 184508 |
| 62503 | 91474874 | 91476027 | 224441, 184508, 140553 |
| 62504 | 91478368 | 91479488 | 140553 |
| 62505 | 91536842 | 91538350 | 198901 |
| 62506 | 91564992 | 91566415 | 214432, 184056 |
| 62507 | 91950420 | 91951195 | |
| 62508 | 91951461 | 91952715 | 258551 |
| 62509 | 91999492 | 92000095 | 258551 |
| 62510 | 92039764 | 92040939 | 258551 |
| 62511 | 92078501 | 92082402 | 258551 |
| 62512 | 92237387 | 92238672 | |
| 62513 | 92395759 | 92397228 | 176463 |
| 62514 | 92485830 | 92486707 | 176463 |
| 62515 | 92660343 | 92660955 | 176463 |
| 62516 | 92707797 | 92708700 | 258761, 176463, 260661 |
| 62517 | 92713741 | 92714944 | 258761, 176463, 260661 |
| 62518 | 93160129 | 93161734 | 185442, 271763 |
| 62519 | 93208395 | 93209013 | 185442 |
| 62520 | 93209979 | 93211059 | 185442 |
| 62521 | 93224309 | 93224887 | 185442 |
| 62522 | 93225877 | 93226491 | 185442 |
| 62523 | 93346344 | 93349376 | 258922, 185442 |
| 62524 | 93350831 | 93355811 | 258922, 185442 |
| 62525 | 93374268 | 93375947 | |
| 62526 | 93424625 | 93429020 | 173575, 272888 |
| 62527 | 93442787 | 93453130 | 264173, 173575 |
| 62528 | 93457003 | 93458602 | 173575 |
| 62529 | 93460704 | 93462219 | 173575 |
| 62530 | 93463082 | 93466362 | 173575 |
| 62531 | 93571147 | 93572559 | 173575 |
| 62532 | 94773595 | 94774903 | 140563 |
| 62533 | 94791106 | 94792307 | 140563 |
| 62534 | 95075379 | 95076380 | |
| 62535 | 96100398 | 96101887 | |
| 62536 | 96163798 | 96164702 | |
| 62537 | 96196667 | 96197298 | |
| 62538 | 96207594 | 96208213 | |
| 62539 | 96223078 | 96223702 | |
| 62540 | 96227466 | 96228829 | |
| 62541 | 96569415 | 96569946 | |
| 62542 | 96596219 | 96597748 | 259702 |
| 62543 | 96705324 | 96706253 | 247809 |
| 62544 | 96713949 | 96716095 | 259359, 247809 |
| 62545 | 96724465 | 96725704 | 247809 |
| 62546 | 96726288 | 96728257 | 247809 |
| 62547 | 96752528 | 96753324 | 247809 |
| 62548 | 96804795 | 96805482 | 247809 |
| 62549 | 96813573 | 96814393 | 247809 |
| 62550 | 96864161 | 96866036 | 247809 |
| 62551 | 96872891 | 96877488 | 222651, 185551 |
| 62552 | 96883277 | 96884975 | 185551 |
| 62553 | 96894609 | 96896022 | |
| 62554 | 96897099 | 96898627 | 259275 |
| 62555 | 96899964 | 96901227 | 259275 |
| 62556 | 96971672 | 96972593 | |
| 62557 | 97432964 | 97433751 | |
| 62558 | 98234450 | 98235593 | |
| 62559 | 98503354 | 98506508 | 140450 |
| 62560 | 98521380 | 98523424 | |
| 62561 | 98524830 | 98525472 | |
| 62562 | 98527210 | 98527931 | 259478 |
| 62563 | 98541263 | 98542375 | |
| 62564 | 98545876 | 98547390 | |
| 62565 | 98552745 | 98554254 | |
| 62566 | 98561873 | 98562832 | |
| 62567 | 98571451 | 98572415 | |
| 62568 | 98578117 | 98579402 | |
| 62569 | 98971457 | 98972322 | |
| 62570 | 99191185 | 99191982 | |
| 62571 | 99602281 | 99602765 | 259475 |
| 62572 | 99619801 | 99620705 | 259475 |
| 62573 | 99671313 | 99672635 | 261054, 182253 |
| 62574 | 99683425 | 99684439 | 261616, 103852 |
| 62575 | 99694213 | 99695041 | 103852 |
| 62576 | 99788803 | 99790119 | 103852 |
| 62577 | 99790123 | 99792433 | 168904, 103852 |
| 62578 | 99797349 | 99798065 | 259706, 264771, 168904 |
| 62579 | 99845990 | 99847668 | 168904 |
| 62580 | 99855747 | 99856389 | 168904 |
| 62581 | 99860386 | 99861635 | 168904 |
| 62582 | 99866515 | 99869356 | 168904 |
| 62583 | 99924875 | 99926666 | 168904 |
| 62584 | 100104872 | 100107940 | 068305 |
| 62585 | 100132109 | 100132661 | 068305 |
| 62586 | 100154319 | 100155458 | 068305 |
| 62587 | 100161981 | 100162545 | 068305 |
| 62588 | 100168455 | 100169334 | 068305 |
| 62589 | 100242795 | 100245245 | 068305 |
| 62590 | 100246914 | 100249723 | 068305 |
| 62591 | 100272419 | 100274474 | 183060 |
| 62592 | 100289228 | 100290395 | |
| 62593 | 100671490 | 100672541 | 140470 |
| 62594 | 100752007 | 100753073 | 140470 |
| 62595 | 100756095 | 100757319 | 140470 |
| 62596 | 100781217 | 100783148 | 140470 |
| 62597 | 100793198 | 100793858 | 140470 |
| 62598 | 100824394 | 100825443 | 140470 |
| 62599 | 100881314 | 100882863 | 140470 |
| 62600 | 100885438 | 100888216 | 189419 |
| 62601 | 100888885 | 100892516 | 189419 |
| 62602 | 100912919 | 100914201 | 259430 |
| 62603 | 101008344 | 101008959 | 154227 |
| 62604 | 101056922 | 101058035 | 154227 |
| 62605 | 101099543 | 101099982 | 140471, 259540 |
| 62606 | 101141336 | 101143685 | 183475, 140471 |
| 62607 | 101255142 | 101256864 | 259579 |
| 62608 | 101260390 | 101263668 | 259579 |
| 62609 | 101548379 | 101549010 | 154237 |
| 62610 | 101791863 | 101793094 | 131873 |
| 62611 | 101816537 | 101818070 | 131871 |
| 62612 | 101834197 | 101836779 | 259172, 131876 |
| 62613 | 101864526 | 101866092 | 140479 |
| 62614 | 101976993 | 101981174 | 140479 |
| 62615 | 101983551 | 101988637 | 140479 |
| 62616 | 101990544 | 101992374 | 140479 |
| 62617 | 101995007 | 101997239 | 140479 |
| 62618 | 102017913 | 102018428 | 140479 |
| 62619 | 102028109 | 102030709 | 140479 |
| 62620 | 102040091 | 102042223 | 140479 |
| 62621 | 102066959 | 102069468 | |
| 62622 | 102153357 | 102155174 | |
| 62623 | 102156024 | 102157138 | |
| 62624 | 102191133 | 102193699 | 184277 |
| 62625 | 102263812 | 102264929 | 185418 |

The locations of ODSCs on Chromosome 16 are shown in Table 58.

TABLE 58
ODSC Locations on Chromosome 16

| SEQ ID NO. | Start | End | Overlapping Gene(s) |
|---|---|---|---|
| 62626 | 102786 | 103908 | 161981, 161980 |
| 62627 | 121815 | 123353 | 007384 |
| 62628 | 127253 | 129287 | 103152 |
| 62629 | 148787 | 149545 | 103148 |
| 62630 | 163041 | 163900 | 103148 |
| 62631 | 187604 | 189118 | 103148 |
| 62632 | 231253 | 232080 | |
| 62633 | 278554 | 279784 | 007392 |
| 62634 | 284321 | 285684 | 167930 |
| 62635 | 377005 | 378231 | 103126 |
| 62636 | 401534 | 402586 | 103126 |
| 62637 | 420488 | 421349 | 129925, 086504 |
| 62638 | 430905 | 432847 | 236829, 129925 |
| 62639 | 446902 | 447787 | 103202 |
| 62640 | 451394 | 453082 | 242612, 103202 |
| 62641 | 455026 | 456688 | 242612, 103202 |
| 62642 | 473670 | 476976 | 090565 |
| 62643 | 551939 | 553769 | 090565 |
| 62644 | 576001 | 579567 | 103326 |
| 62645 | 583460 | 584689 | 103326 |
| 62646 | 585074 | 586508 | 266124, 103326 |
| 62647 | 592911 | 594033 | 266235, 103326 |
| 62648 | 619236 | 621676 | 257108, 007541 |
| 62649 | 637638 | 641287 | 197562 |
| 62650 | 641845 | 643923 | 197562 |
| 62651 | 685579 | 687101 | 130731 |
| 62652 | 691148 | 693569 | 172366, 197727 |
| 62653 | 695213 | 700217 | 161996, 228201, 172366 |
| 62654 | 717530 | 719029 | 140983, 161996 |
| 62655 | 725474 | 726203 | 103269 |
| 62656 | 730061 | 731375 | 103266 |
| 62657 | 733343 | 735074 | 127580, 161999 |
| 62658 | 739462 | 740693 | 127580, 261659 |
| 62659 | 762519 | 763162 | |
| 62660 | 765014 | 766551 | 103260 |
| 62661 | 769858 | 771683 | 103254 |
| 62662 | 789828 | 791520 | 103245 |
| 62663 | 837720 | 838678 | 127586, 007376 |
| 62664 | 856942 | 858587 | 167945 |
| 62665 | 1019966 | 1021159 | 103227 |
| 62666 | 1128441 | 1128959 | 261713, 162009 |
| 62667 | 1202419 | 1203010 | |
| 62668 | 1203802 | 1204996 | 196557 |
| 62669 | 1209790 | 1210863 | 259910, 196557 |
| 62670 | 1224402 | 1225365 | 196557 |
| 62671 | 1231837 | 1233192 | 196557 |
| 62672 | 1234398 | 1234951 | 196557 |
| 62673 | 1340777 | 1342029 | |
| 62674 | 1346821 | 1350515 | 260710 |
| 62675 | 1358249 | 1361242 | 103275 |
| 62676 | 1393018 | 1393689 | 007516 |
| 62677 | 1400966 | 1402945 | 090581, 007520 |
| 62678 | 1427903 | 1430349 | 265529, 059145 |
| 62679 | 1463582 | 1464905 | 059145 |
| 62680 | 1469739 | 1471885 | 174109 |
| 62681 | 1524118 | 1526025 | 103249 |
| 62682 | 1542487 | 1544275 | 100726 |
| 62683 | 1660007 | 1661620 | 260989, 187535 |
| 62684 | 1661853 | 1663302 | 007545, 187535 |
| 62685 | 1663634 | 1665420 | 007545 |
| 62686 | 1700888 | 1702265 | 261732, 007545 |
| 62687 | 1727908 | 1729664 | 206053, 007545, 261732 |
| 62688 | 1737244 | 1743051 | 206053 |
| 62689 | 1755245 | 1757307 | 138834 |
| 62690 | 1772101 | 1772571 | 138834 |
| 62691 | 1820402 | 1824237 | 197774, 074071, 103024 |
| 62692 | 1831038 | 1833845 | 095906, 197774, 162032 |
| 62693 | 1843192 | 1845230 | 099769, 162032 |
| 62694 | 1875250 | 1879718 | 180185, 063854 |
| 62695 | 1880021 | 1880915 | 180185 |
| 62696 | 1919686 | 1921584 | 162039 |
| 62697 | 1979153 | 1980211 | |
| 62698 | 1990166 | 1993684 | 198736 |
| 62699 | 2009096 | 2010443 | 140990 |
| 62700 | 2013711 | 2016047 | 255198, 255513, 140988 |
| 62701 | 2021040 | 2023041 | 183751 |
| 62702 | 2033466 | 2035207 | 127554, 261790, 196408 |
| 62703 | 2058141 | 2061005 | 183971, 167962 |
| 62704 | 2076711 | 2078694 | 065054 |
| 62705 | 2082506 | 2084433 | 065054 |
| 62706 | 2084653 | 2086701 | 065054 |
| 62707 | 2088043 | 2091576 | 065057, 065054 |
| 62708 | 2097086 | 2098908 | 103197, 065057 |
| 62709 | 2197344 | 2200212 | 167964 |
| 62710 | 2202512 | 2208950 | 131653, 206630, 260260, |
| | | | 167964 |
| 62711 | 2210543 | 2212222 | 131653 |
| 62712 | 2254813 | 2256606 | 252958, 167965 |
| 62713 | 2263668 | 2265935 | 184207 |
| 62714 | 2273018 | 2274496 | 167967 |
| 62715 | 2276259 | 2276917 | 167967 |
| 62716 | 2300085 | 2302628 | 167969 |
| 62717 | 2317604 | 2319033 | 207715, 260778, 205937 |
| 62718 | 2389544 | 2391397 | 167972, 238098 |
| 62719 | 2438760 | 2439392 | 238098 |
| 62720 | 2479204 | 2479920 | 162063 |
| 62721 | 2517779 | 2519617 | 259895 |
| 62722 | 2521136 | 2522754 | 162068 |
| 62723 | 2523715 | 2526217 | 162065, 162068 |
| 62724 | 2547079 | 2547889 | 260272, 162065 |
| 62725 | 2563432 | 2565504 | 259784, 185883, 260272 |
| 62726 | 2570056 | 2571271 | 162066, 185883, 260272, |
| | | | 259784 |
| 62727 | 2581230 | 2583702 | 266232, 162066, 205923 |
| 62728 | 2586990 | 2588303 | 140992 |
| 62729 | 2652953 | 2654331 | 215154, 140992 |
| 62730 | 2723165 | 2724095 | 260565 |
| 62731 | 2731588 | 2733359 | 167977 |
| 62732 | 2768783 | 2769886 | 172382 |
| 62733 | 2770535 | 2771931 | 172382 |
| 62734 | 2801359 | 2804068 | 167978, 205913 |
| 62735 | 2825855 | 2828648 | 103363 |
| 62736 | 2885171 | 2887169 | 162078 |
| 62737 | 2917496 | 2918873 | 263280 |
| 62738 | 2932869 | 2933990 | 162076 |
| 62739 | 2954070 | 2955817 | |
| 62740 | 2961613 | 2962983 | 059122 |
| 62741 | 3027278 | 3027895 | 127564 |
| 62742 | 3030407 | 3031376 | 127564 |
| 62743 | 3053711 | 3057933 | 272079, 262362 |
| 62744 | 3069962 | 3074692 | 131652, 103145, 184697, |
| | | | 006327 |
| 62745 | 3108074 | 3110694 | 261971, 008516 |
| 62746 | 3114363 | 3116944 | 008517 |
| 62747 | 3117009 | 3118263 | 008517 |
| 62748 | 3149430 | 3150900 | |
| 62749 | 3155681 | 3157026 | 263011 |
| 62750 | 3162046 | 3163570 | 122386, 263214 |
| 62751 | 3183771 | 3185856 | 263072, 085644 |
| 62752 | 3193496 | 3194424 | 228146 |
| 62753 | 3199653 | 3201337 | 228146 |
| 62754 | 3202337 | 3203375 | |
| 62755 | 3206942 | 3209451 | 261889 |
| 62756 | 3221830 | 3222641 | |
| 62757 | 3284212 | 3285731 | 010539 |
| 62758 | 3309040 | 3309789 | |
| 62759 | 3313279 | 3315154 | 006194 |
| 62760 | 3332111 | 3334446 | 006194 |
| 62761 | 3354580 | 3356431 | 162086, 140993 |
| 62762 | 3414640 | 3415529 | 262621 |
| 62763 | 3450123 | 3452125 | 103343, 140987, 262621 |
| 62764 | 3492981 | 3494371 | 122390, 167981, 262621 |
| 62765 | 3506924 | 3509666 | 263212, 122390, 262621 |
| 62766 | 3550640 | 3551723 | 103351, 263212 |
| 62767 | 3660844 | 3662454 | 213918, 188827 |
| 62768 | 3696152 | 3696816 | 213918 |
| 62769 | 3744737 | 3747335 | 126602 |
| 62770 | 3766511 | 3768577 | 126602 |
| 62771 | 3907223 | 3907860 | 005339 |
| 62772 | 3929184 | 3931704 | 005339 |
| 62773 | 4009203 | 4009704 | 162104 |
| 62774 | 4010048 | 4011254 | 162104 |
| 62775 | 4061865 | 4062892 | 162104 |
| 62776 | 4064850 | 4065893 | 162104 |
| 62777 | 4069295 | 4069788 | 162104 |
| 62778 | 4103071 | 4104808 | 162104 |
| 62779 | 4109825 | 4110781 | 162104 |
| 62780 | 4114508 | 4115800 | 162104 |
| 62781 | 4141342 | 4142159 | 162104 |
| 62782 | 4143964 | 4144607 | 162104 |
| 62783 | 4165258 | 4167093 | 162104 |
| 62784 | 4189871 | 4192021 | |
| 62785 | 4232774 | 4234880 | 263105 |
| 62786 | 4262361 | 4266268 | 185739 |
| 62787 | 4303119 | 4304447 | 262468 |
| 62788 | 4321049 | 4324219 | 090447 |
| 62789 | 4340183 | 4341265 | |
| 62790 | 4356574 | 4358274 | |
| 62791 | 4363814 | 4368478 | 126603 |
| 62792 | 4377915 | 4378779 | 262686, 126603 |
| 62793 | 4378843 | 4379322 | 126603 |
| 62794 | 4395672 | 4396545 | 103426, 217930 |
| 62795 | 4400320 | 4402234 | 103426, 217930 |
| 62796 | 4420856 | 4428142 | 168140, 103426, 262246 |
| 62797 | 4465165 | 4467140 | 103426, 262246 |
| 62798 | 4474519 | 4477061 | 103423, 262246 |
| 62799 | 4524459 | 4524963 | 103415, 153406 |
| 62800 | 4526061 | 4527341 | 153406, 103415 |
| 62801 | 4538036 | 4538683 | 153406, 103415 |
| 62802 | 4586609 | 4589162 | 089486 |
| 62803 | 4663490 | 4665586 | 153443 |
| 62804 | 4665922 | 4667426 | 102858 |
| 62805 | 4668675 | 4669752 | 102858 |
| 62806 | 4670439 | 4671071 | 102858 |
| 62807 | 4674119 | 4676719 | 102858 |
| 62808 | 4703636 | 4705197 | 102858 |
| 62809 | 4713186 | 4715649 | 102858 |
| 62810 | 4724958 | 4726354 | 102858 |
| 62811 | 4728775 | 4731290 | 102858 |
| 62812 | 4733640 | 4735233 | 102858 |
| 62813 | 4738107 | 4738598 | 102858 |
| 62814 | 4742756 | 4744423 | 168101 |
| 62815 | 4752178 | 4752898 | 168096 |
| 62816 | 4783657 | 4784604 | 166246, 168096, 266994 |
| 62817 | 4816398 | 4817641 | 103199 |
| 62818 | 4818085 | 4819406 | |
| 62819 | 4896297 | 4898593 | 118900, 140632 |
| 62820 | 4912530 | 4913071 | 118900 |
| 62821 | 4956092 | 4956986 | 118898 |
| 62822 | 4974792 | 4975548 | 118898 |
| 62823 | 4985097 | 4985891 | 118898 |
| 62824 | 4985896 | 4988104 | 118898 |
| 62825 | 4994172 | 4996518 | 118898 |
| 62826 | 5007377 | 5008541 | 103184, 118898 |
| 62827 | 5082628 | 5084678 | 033011, 103174, 267072 |
| 62828 | 5121096 | 5122908 | 033011 |
| 62829 | 8223044 | 8223695 | |
| 62830 | 8420565 | 8421108 | 259819 |
| 62831 | 8715173 | 8716774 | 067365 |
| 62832 | 8717487 | 8718052 | 067365 |
| 62833 | 8767595 | 8770101 | 183044 |
| 62834 | 8772416 | 8773984 | 183044 |
| 62835 | 8823940 | 8824844 | 183044 |
| 62836 | 8833981 | 8835319 | 183044 |
| 62837 | 8845642 | 8846827 | 183044 |
| 62838 | 8890580 | 8893074 | 184857, 140650 |
| 62839 | 8943735 | 8944942 | 260276 |
| 62840 | 8953858 | 8954540 | 260276, 153048 |
| 62841 | 8955901 | 8958457 | 153048 |
| 62842 | 8961280 | 8963341 | 261481, 153048 |
| 62843 | 8972044 | 8972890 | |
| 62844 | 9055676 | 9057370 | 260979, 187555 |
| 62845 | 9057516 | 9058570 | 187555, 260979 |
| 62846 | 9080864 | 9081704 | |
| 62847 | 9140726 | 9142841 | |
| 62848 | 9184027 | 9187549 | 182831 |
| 62849 | 9192204 | 9193586 | 182831 |
| 62850 | 9206047 | 9206818 | 182831, 263244 |
| 62851 | 9208515 | 9209675 | 182831 |
| 62852 | 9217704 | 9218495 | |
| 62853 | 9221208 | 9222372 | |
| 62854 | 9334991 | 9335441 | |
| 62855 | 9352998 | 9354863 | |
| 62856 | 9401913 | 9402879 | |
| 62857 | 9444523 | 9446575 | |
| 62858 | 9458081 | 9460653 | 261617 |
| 62859 | 9472445 | 9473317 | 261617 |
| 62860 | 9516429 | 9517167 | |
| 62861 | 9532695 | 9533279 | |
| 62862 | 9635362 | 9636843 | |
| 62863 | 9638880 | 9640584 | |
| 62864 | 9741868 | 9742632 | |
| 62865 | 10215102 | 10216090 | 183454 |
| 62866 | 10350551 | 10351501 | |
| 62867 | 10359612 | 10360290 | |
| 62868 | 10363482 | 10364658 | |
| 62869 | 10406449 | 10407599 | |
| 62870 | 10427053 | 10430069 | 166669 |
| 62871 | 10434986 | 10437050 | 166669 |
| 62872 | 10460268 | 10461172 | 166669 |
| 62873 | 10461227 | 10462122 | 166669 |
| 62874 | 10479433 | 10481018 | 166669 |
| 62875 | 10603044 | 10603951 | |
| 62876 | 10608191 | 10609330 | 260468 |
| 62877 | 10613377 | 10615142 | 260468 |
| 62878 | 10630523 | 10631182 | 213853 |
| 62879 | 10652055 | 10653473 | 213853 |
| 62880 | 10673648 | 10674766 | 213853 |
| 62881 | 10695443 | 10696179 | |
| 62882 | 10710189 | 10711298 | |
| 62883 | 10740470 | 10740945 | 153060 |
| 62884 | 10741450 | 10742225 | 153060 |
| 62885 | 10823508 | 10823912 | |
| 62886 | 10830301 | 10833008 | |
| 62887 | 10837099 | 10838391 | 103274 |
| 62888 | 10921371 | 10923650 | |
| 62889 | 10972500 | 10973520 | 262151, 179583 |
| 62890 | 11009499 | 11010409 | 179583 |
| 62891 | 11012437 | 11013066 | 179583 |
| 62892 | 11014582 | 11016782 | 179583 |
| 62893 | 11017011 | 11018152 | 179583 |
| 62894 | 11032166 | 11033501 | 263013, 182108 |
| 62895 | 11034466 | 11035518 | 262222, 182108 |
| 62896 | 11038086 | 11039266 | 038532 |
| 62897 | 11059172 | 11059889 | 038532 |
| 62898 | 11073656 | 11074386 | 038532 |
| 62899 | 11085509 | 11086085 | 038532 |
| 62900 | 11117460 | 11119128 | 038532 |
| 62901 | 11161351 | 11162055 | 038532, 262020 |
| 62902 | 11173181 | 11173759 | 038532 |
| 62903 | 11198604 | 11199704 | 038532 |
| 62904 | 11226610 | 11227561 | 038532 |
| 62905 | 11298692 | 11300942 | 263033 |
| 62906 | 11349218 | 11351383 | 185338, 175643 |
| 62907 | 11352498 | 11353724 | 175643 |
| 62908 | 11373223 | 11373853 | 175643 |
| 62909 | 11438880 | 11439911 | 263080, 175643 |
| 62910 | 11456939 | 11457647 | 188897 |
| 62911 | 11588671 | 11590407 | 188897 |
| 62912 | 11590671 | 11592295 | 188897 |
| 62913 | 11627850 | 11630356 | |
| 62914 | 11644410 | 11645328 | 189067 |
| 62915 | 11647886 | 11648533 | 189067 |
| 62916 | 11679585 | 11681628 | 189067 |
| 62917 | 11705790 | 11707128 | 189067 |
| 62918 | 11733261 | 11735667 | |
| 62919 | 11762242 | 11763074 | 184602 |
| 62920 | 11769208 | 11770250 | 184602 |
| 62921 | 11794022 | 11795073 | 153066 |
| 62922 | 11835177 | 11838188 | 262420, 153066 |
| 62923 | 11841992 | 11843041 | |
| 62924 | 11876737 | 11878205 | 122299 |
| 62925 | 11889852 | 11892711 | 122299 |
| 62926 | 11927867 | 11929276 | 171490 |
| 62927 | 11944183 | 11946444 | 263307, 171490 |
| 62928 | 11961190 | 11961722 | 263307 |
| 62929 | 11963377 | 11964318 | 103342, 263307 |
| 62930 | 12008257 | 12010982 | 266163, 103342 |
| 62931 | 12069494 | 12071845 | 048471, 260488, 234719 |
| 62932 | 12118288 | 12118847 | 048471 |
| 62933 | 12156503 | 12157117 | 048471 |
| 62934 | 12191214 | 12191929 | 048471 |
| 62935 | 12235577 | 12236937 | 048471 |
| 62936 | 12280178 | 12283239 | 048471 |
| 62937 | 12304638 | 12306765 | 048471 |
| 62938 | 12351761 | 12352501 | 048471 |
| 62939 | 12408894 | 12410060 | 048471 |
| 62940 | 12500712 | 12501735 | 048471 |
| 62941 | 12625198 | 12626640 | 048471 |
| 62942 | 12664025 | 12664637 | 048471, 259899 |
| 62943 | 12703187 | 12710151 | 259899 |
| 62944 | 12736865 | 12742238 | |
| 62945 | 12742338 | 12748076 | |
| 62946 | 12846793 | 12849253 | 260378, 103381 |
| 62947 | 12850932 | 12852233 | 260378, 103381 |
| 62948 | 12864635 | 12865939 | 103381 |
| 62949 | 12896330 | 12898153 | 103381 |
| 62950 | 13177875 | 13178700 | 237515 |
| 62951 | 13202737 | 13206097 | 237515 |
| 62952 | 13338966 | 13341815 | 262801 |
| 62953 | 13363142 | 13364429 | 262801 |
| 62954 | 13400627 | 13401873 | 262801 |
| 62955 | 13557395 | 13558319 | 262801 |
| 62956 | 13887885 | 13888590 | |
| 62957 | 13888854 | 13889605 | |
| 62958 | 13959842 | 13960535 | |
| 62959 | 14003393 | 14005519 | |
| 62960 | 14007087 | 14007947 | |
| 62961 | 14029817 | 14032490 | 175595 |
| 62962 | 14050694 | 14052749 | |
| 62963 | 14088172 | 14089520 | |
| 62964 | 14107205 | 14108307 | 262097 |
| 62965 | 14144161 | 14145289 | |
| 62966 | 14164324 | 14166222 | 186260 |
| 62967 | 14181736 | 14182961 | 186260 |
| 62968 | 14199725 | 14200794 | 186260 |
| 62969 | 14206005 | 14206616 | 186260 |
| 62970 | 14245188 | 14245994 | 186260 |
| 62971 | 14266150 | 14266832 | 186260 |
| 62972 | 14334057 | 14334868 | 186260 |
| 62973 | 14378722 | 14380650 | |
| 62974 | 14394638 | 14398598 | 207639, 262454 |
| 62975 | 14400386 | 14402620 | 262454 |
| 62976 | 14403994 | 14404833 | 262454 |
| 62977 | 14431279 | 14432006 | |
| 62978 | 14438715 | 14440097 | |
| 62979 | 14493255 | 14494304 | |
| 62980 | 14579755 | 14582244 | 140694 |
| 62981 | 14591954 | 14592905 | 140694 |
| 62982 | 14594301 | 14601083 | 140694 |
| 62983 | 14601476 | 14606538 | 140694 |
| 62984 | 14620209 | 14623822 | 140694 |
| 62985 | 14638823 | 14640277 | 140694 |
| 62986 | 14642820 | 14644174 | 140694 |
| 62987 | 14650106 | 14650702 | 140694 |
| 62988 | 14653347 | 14654832 | 140694 |
| 62989 | 14656147 | 14659332 | 140694 |
| 62990 | 14670190 | 14670934 | 140694 |
| 62991 | 14720808 | 14721406 | 140694 |
| 62992 | 14723299 | 14724514 | 140694 |
| 62993 | 14725689 | 14728345 | 103429, 140694 |
| 62994 | 15145851 | 15146391 | 179889, 157045 |
| 62995 | 15148918 | 15150516 | 157045, 179889 |
| 62996 | 15489587 | 15490096 | 156968, 261130 |
| 62997 | 15492198 | 15493476 | 156968, 261130 |
| 62998 | 15498644 | 15499149 | 156968, 261130 |
| 62999 | 15528272 | 15529985 | 166780, 261130 |
| 63000 | 15537681 | 15538396 | 166780, 261130 |
| 63001 | 15549496 | 15549950 | 166780, 261130 |
| 63002 | 15575747 | 15576818 | 166780, 261130 |
| 63003 | 15582562 | 15584865 | 166780, 261130 |
| 63004 | 15587009 | 15589100 | 166780, 261130 |
| 63005 | 15669813 | 15672407 | 166780 |
| 63006 | 15684035 | 15685180 | 166780 |
| 63007 | 15701124 | 15703015 | 257769, 166783, 166780 |
| 63008 | 15722722 | 15723442 | 166783 |
| 63009 | 15735760 | 15738522 | 272213, 072864, 166783 |
| 63010 | 15743776 | 15744781 | 072864 |
| 63011 | 15766446 | 15767236 | 072864 |
| 63012 | 15981410 | 15983037 | 133393 |
| 63013 | 15993498 | 15994210 | |
| 63014 | 16090239 | 16091106 | 103222 |
| 63015 | 16108533 | 16109141 | 103222 |
| 63016 | 16113269 | 16113790 | 103222 |
| 63017 | 16115605 | 16117137 | 103222 |
| 63018 | 16119446 | 16120008 | 103222 |
| 63019 | 16153607 | 16154389 | 103222 |
| 63020 | 16237536 | 16238778 | |
| 63021 | 16243232 | 16244702 | 091262 |
| 63022 | 16951593 | 16952313 | |
| 63023 | 17265014 | 17265905 | 103489 |
| 63024 | 17266716 | 17268098 | 103489 |
| 63025 | 17862124 | 17862945 | |
| 63026 | 18010428 | 18012283 | |
| 63027 | 18040035 | 18042268 | 259929 |
| 63028 | 18800577 | 18802097 | 134419, 260342 |
| 63029 | 18811827 | 18813550 | 260342, 170540 |
| 63030 | 18957076 | 18958076 | |
| 63031 | 18994922 | 18996026 | 170537 |
| 63032 | 19008931 | 19010553 | 170537 |
| 63033 | 19078532 | 19079990 | 167186, 261465 |
| 63034 | 19097597 | 19098351 | 260430 |
| 63035 | 19118900 | 19119427 | |
| 63036 | 19122114 | 19123005 | |
| 63037 | 19124372 | 19126425 | 205730, 261427 |
| 63038 | 19134485 | 19135515 | 261427 |
| 63039 | 19178802 | 19180480 | 103528, 261427 |
| 63040 | 19193639 | 19196836 | 103528 |
| 63041 | 19201672 | 19202789 | 103528 |
| 63042 | 19214410 | 19215952 | 103528 |
| 63043 | 19246953 | 19249056 | 103528 |
| 63044 | 19295007 | 19296241 | |
| 63045 | 19341207 | 19343037 | |
| 63046 | 19532661 | 19533647 | 006007 |
| 63047 | 19535064 | 19536207 | 103540 |
| 63048 | 19553330 | 19553801 | 103540 |
| 63049 | 19566026 | 19567935 | 103544 |
| 63050 | 19711491 | 19712499 | 103544 |
| 63051 | 19728604 | 19730230 | 103550, 174628 |
| 63052 | 19818831 | 19824563 | 174628 |
| 63053 | 19828044 | 19828959 | 174628 |
| 63054 | 19847074 | 19848162 | 174628 |
| 63055 | 19894512 | 19898081 | 167191 |
| 63056 | 20396952 | 20398248 | 263237, 169340 |
| 63057 | 20420525 | 20422704 | 183549 |
| 63058 | 20449233 | 20449915 | 183549 |
| 63059 | 20622589 | 20623490 | 005187 |
| 63060 | 20684745 | 20685528 | 005187, 166743 |
| 63061 | 20751890 | 20753608 | 066654, 005187 |
| 63062 | 20774804 | 20775578 | 260510, 005187 |
| 63063 | 20778781 | 20779868 | 005187 |
| 63064 | 20817041 | 20818841 | 005189, 196678 |
| 63065 | 20862041 | 20863825 | 196678 |
| 63066 | 20876295 | 20878965 | 188215, 196678 |
| 63067 | 20889735 | 20891435 | 188215, 196678 |
| 63068 | 20894833 | 20895377 | 188215, 196678 |
| 63069 | 20908533 | 20913251 | 102897, 188215, 196678 |
| 63070 | 20916997 | 20918758 | 102897 |
| 63071 | 20922076 | 20923716 | 102897 |
| 63072 | 20929969 | 20930606 | 102897 |
| 63073 | 21071235 | 21071812 | 158486 |
| 63074 | 21199713 | 21200387 | |
| 63075 | 21244462 | 21246183 | 175311 |
| 63076 | 21271732 | 21273870 | 103316 |
| 63077 | 21275008 | 21277890 | 103316 |
| 63078 | 21289294 | 21290141 | 103316 |
| 63079 | 21311681 | 21313653 | 189149, 103316 |
| 63080 | 21609747 | 21612074 | 197006 |
| 63081 | 21831446 | 21832146 | 248124 |
| 63082 | 21963610 | 21965650 | 140740 |
| 63083 | 22012198 | 22012883 | 155714 |
| 63084 | 22018457 | 22020524 | 185716 |
| 63085 | 22022292 | 22022896 | 185716 |
| 63086 | 22025699 | 22026351 | 185716 |
| 63087 | 22037769 | 22038436 | 185716 |
| 63088 | 22199421 | 22200431 | 183921 |
| 63089 | 22201174 | 22204555 | 183921 |
| 63090 | 22206661 | 22207891 | |
| 63091 | 22216639 | 22219016 | 103319 |
| 63092 | 22243797 | 22244918 | 103319 |
| 63093 | 22308158 | 22310168 | 058600 |
| 63094 | 22364395 | 22365951 | 140743 |
| 63095 | 22379791 | 22381344 | 260635, 140743 |
| 63096 | 22382366 | 22383935 | 140743 |
| 63097 | 22384839 | 22386437 | 260790, 140743 |
| 63098 | 23115158 | 23117065 | 103404 |
| 63099 | 23156348 | 23158657 | 103404 |
| 63100 | 23159139 | 23161797 | 103404 |
| 63101 | 23463385 | 23464894 | 260136, 168434 |
| 63102 | 23480737 | 23481778 | 103365 |
| 63103 | 23508425 | 23509495 | 103365 |
| 63104 | 23515871 | 23516609 | 103365 |
| 63105 | 23521070 | 23522070 | 103365 |
| 63106 | 23549347 | 23551083 | 103356 |
| 63107 | 23567896 | 23570056 | 103353, 103356 |
| 63108 | 23606384 | 23607928 | 004779 |
| 63109 | 23651718 | 23653566 | 166847, 083093 |
| 63110 | 24545471 | 24546510 | |
| 63111 | 24550264 | 24553104 | 122257 |
| 63112 | 24657206 | 24658685 | |
| 63113 | 24679749 | 24681279 | 224310 |
| 63114 | 24711457 | 24713719 | |
| 63115 | 24717053 | 24718334 | |
| 63116 | 24740091 | 24742403 | 090905 |
| 63117 | 24742581 | 24743963 | 090905 |
| 63118 | 24747359 | 24749510 | 090905 |
| 63119 | 24855226 | 24856396 | |
| 63120 | 24986107 | 24988338 | 140750 |
| 63121 | 24988504 | 24989770 | 140750 |
| 63122 | 24990966 | 24992606 | 140750 |
| 63123 | 24997798 | 24998992 | 140750 |
| 63124 | 24999045 | 25001246 | 140750 |
| 63125 | 25004848 | 25007140 | 140750 |
| 63126 | 25017557 | 25018529 | 140750 |
| 63127 | 25023860 | 25024398 | 140750 |
| 63128 | 25025114 | 25027296 | 140750 |
| 63129 | 25117270 | 25119257 | 260448 |
| 63130 | 25120810 | 25121632 | 260448 |
| 63131 | 25121749 | 25123842 | 205629, 260448 |
| 63132 | 25188717 | 25190198 | 205629 |
| 63133 | 25268549 | 25270362 | 155592 |
| 63134 | 25644651 | 25645298 | |
| 63135 | 26541738 | 26542479 | |
| 63136 | 27190727 | 27191264 | |
| 63137 | 27200379 | 27201268 | |
| 63138 | 27214423 | 27216303 | 155666 |
| 63139 | 27219481 | 27220855 | 155666 |
| 63140 | 27224817 | 27229663 | 259940, 155666 |
| 63141 | 27233478 | 27234852 | |
| 63142 | 27264249 | 27265180 | 169189 |
| 63143 | 27269606 | 27270071 | 169189 |
| 63144 | 27272824 | 27273464 | 169189 |
| 63145 | 27279260 | 27280954 | 245888, 169189 |
| 63146 | 27284513 | 27286074 | 245888 |
| 63147 | 27324507 | 27328188 | 077238 |
| 63148 | 27333809 | 27335015 | 077238 |
| 63149 | 27337539 | 27339355 | 077238 |
| 63150 | 27375017 | 27377369 | 077238 |
| 63151 | 27560427 | 27562178 | 047578, 077235 |
| 63152 | 27589198 | 27591254 | 047578 |
| 63153 | 28192231 | 28193378 | 169180 |
| 63154 | 28222157 | 28224069 | 169180 |
| 63155 | 28288831 | 28289935 | |
| 63156 | 28303201 | 28303893 | 188322, 246465 |
| 63157 | 28502079 | 28504253 | 261832, 188603 |
| 63158 | 28535182 | 28536924 | |
| 63159 | 28537255 | 28538726 | |
| 63160 | 28543082 | 28544136 | |
| 63161 | 28548314 | 28551046 | 176046 |
| 63162 | 28552183 | 28552752 | |
| 63163 | 28564913 | 28565765 | 176476 |
| 63164 | 28607704 | 28608676 | 197165 |
| 63165 | 28620841 | 28621867 | 196502 |
| 63166 | 28833024 | 28836691 | 168488, 260796 |
| 63167 | 28856783 | 28858811 | 178188, 178952 |
| 63168 | 28874116 | 28876331 | 261766, 178188 |
| 63169 | 28936079 | 28937741 | 177548 |
| 63170 | 28956710 | 28957402 | |
| 63171 | 28961745 | 28963487 | 176953 |
| 63172 | 28985148 | 28987415 | 260367, 169682, 261067 |
| 63173 | 29125984 | 29127061 | 103472 |
| 63174 | 29689832 | 29691586 | 103485 |
| 63175 | 29705504 | 29706562 | 103485 |
| 63176 | 29769214 | 29769806 | |
| 63177 | 29776481 | 29777446 | |
| 63178 | 29801045 | 29802747 | 079616 |
| 63179 | 29815797 | 29817620 | 103495, 079616 |
| 63180 | 29817694 | 29819094 | 259952, 103495 |
| 63181 | 29822345 | 29823727 | 167371, 103495, 238045, |
| | | | 261962 |
| 63182 | 29826776 | 29828894 | 263136, 185928, 167371, |
| | | | 261962 |
| 63183 | 29831572 | 29834124 | 013364, 261962, 261690, |
| | | | 185928 |
| 63184 | 29840357 | 29841436 | 013364, 185928 |
| 63185 | 29872158 | 29873137 | 103502 |
| 63186 | 29873579 | 29875321 | 214725, 103502 |
| 63187 | 29936959 | 29938496 | 247735, 174943 |
| 63188 | 29972860 | 29974453 | 149932 |
| 63189 | 29984765 | 29986240 | 149930 |
| 63190 | 30006535 | 30008304 | 169592, 149929 |
| 63191 | 30074803 | 30078835 | 149925 |
| 63192 | 30086444 | 30089906 | 149923 |
| 63193 | 30106781 | 30108429 | 250616, 090238 |
| 63194 | 30133475 | 30135318 | 102882 |
| 63195 | 30135557 | 30136020 | |
| 63196 | 30363233 | 30364076 | 169217 |
| 63197 | 30365524 | 30368639 | 169221, 260219, 169217 |
| 63198 | 30379570 | 30382433 | 180209, 169221 |
| 63199 | 30388729 | 30390042 | 180096, 180035, 270466, |
| | | | 180209 |
| 63200 | 30405857 | 30407578 | 180096, 180035 |
| 63201 | 30411735 | 30412315 | |
| 63202 | 30418082 | 30419329 | 179965 |
| 63203 | 30420677 | 30421341 | 179965 |
| 63204 | 30428407 | 30430327 | 179965 |
| 63205 | 30440857 | 30442098 | 179958, 179965 |
| 63206 | 30454772 | 30458547 | 179918 |
| 63207 | 30458695 | 30459560 | |
| 63208 | 30500040 | 30500827 | 261346, 005844 |
| 63209 | 30533395 | 30534033 | 005844 |
| 63210 | 30536950 | 30538921 | 169955, 261459, 169957 |
| 63211 | 30545089 | 30547600 | 235560, 260869, 169955, |
| | | | 261459 |
| 63212 | 30568898 | 30570335 | 169951, 260869 |
| 63213 | 30578440 | 30579021 | |
| 63214 | 30582074 | 30583824 | 239791, 229809 |
| 63215 | 30596015 | 30597859 | 260167, 197162 |
| 63216 | 30614713 | 30616333 | 260167, 156853 |
| 63217 | 30620381 | 30622149 | 156853 |
| 63218 | 30644759 | 30646838 | |
| 63219 | 30661072 | 30663583 | 156858 |
| 63220 | 30668184 | 30672098 | 156860 |
| 63221 | 30708713 | 30711389 | 261840, 080603 |
| 63222 | 30724837 | 30725382 | 080603 |
| 63223 | 30751604 | 30754047 | 260899, 080603 |
| 63224 | 30756102 | 30757007 | 260899 |
| 63225 | 30758701 | 30760304 | 156873, 260899 |
| 63226 | 30786200 | 30787826 | 103549 |
| 63227 | 30797598 | 30799397 | 102870 |
| 63228 | 30816187 | 30818267 | |
| 63229 | 30825038 | 30826025 | |
| 63230 | 30855666 | 30856882 | 099385 |
| 63231 | 30885601 | 30887958 | 260083, 262721, 265991, |
| | | | 099385 |
| 63232 | 30904608 | 30905960 | 211591, 260083, 262721, |
| | | | 099385 |
| 63233 | 30912937 | 30914310 | 150281 |
| 63234 | 30932889 | 30935718 | 099364, 260852 |
| 63235 | 30959597 | 30962513 | 261487, 175938, 099364 |
| 63236 | 30967723 | 30970095 | 099381, 261487, 175938 |
| 63237 | 30996102 | 30998387 | 099377, 268863, 099381 |
| 63238 | 31005766 | 31011309 | 099365 |
| 63239 | 31043752 | 31045877 | 103496 |
| 63240 | 31063258 | 31064604 | |
| 63241 | 31083745 | 31086647 | 167395, 167394 |
| 63242 | 31104441 | 31106383 | 255439, 167397 |
| 63243 | 31116935 | 31117876 | 103507 |
| 63244 | 31119325 | 31120560 | 252809, 103507 |
| 63245 | 31121899 | 31122393 | 103507 |
| 63246 | 31128341 | 31130211 | 262766, 103510 |
| 63247 | 31146016 | 31147231 | 052344 |
| 63248 | 31152664 | 31154265 | 178226 |
| 63249 | 31190542 | 31192333 | 089280 |
| 63250 | 31213403 | 31214415 | 261359, 103490 |
| 63251 | 31276135 | 31277891 | 169896 |
| 63252 | 31280766 | 31281375 | 169896 |
| 63253 | 31396867 | 31398369 | |
| 63254 | 31438754 | 31440166 | 156885 |
| 63255 | 31453679 | 31454792 | 176723 |
| 63256 | 31463587 | 31464376 | 261474 |
| 63257 | 31464618 | 31465456 | 261474 |
| 63258 | 31468853 | 31471607 | 140691, 260267 |
| 63259 | 31473268 | 31474282 | 140691 |
| 63260 | 31487794 | 31489704 | 140682 |
| 63261 | 31512786 | 31513581 | 140688 |
| 63262 | 31518673 | 31520614 | 260625, 140688 |
| 63263 | 31711556 | 31712968 | 131797, 260568 |
| 63264 | 31724315 | 31725630 | 197302 |
| 63265 | 31884546 | 31887591 | 185947 |
| 63266 | 31887914 | 31890356 | 185947 |
| 63267 | 33963004 | 33963580 | |
| 63268 | 33963714 | 33964328 | |
| 63269 | 33964674 | 33965179 | |
| 63270 | 34793790 | 34794710 | |
| 63271 | 34795747 | 34796492 | |
| 63272 | 46722346 | 46723369 | 069329 |
| 63273 | 46745113 | 46745518 | 140795 |
| 63274 | 46823393 | 46824358 | 260782, 140795 |
| 63275 | 46843891 | 46844195 | 155330 |
| 63276 | 46863097 | 46865644 | 155330 |
| 63277 | 46873403 | 46874672 | |
| 63278 | 46897359 | 46898869 | |
| 63279 | 46910264 | 46911095 | |
| 63280 | 46916106 | 46920665 | 166123 |
| 63281 | 46952641 | 46953500 | 166123 |
| 63282 | 46954334 | 46955046 | 166123 |
| 63283 | 46982283 | 46983770 | |
| 63284 | 47006431 | 47009008 | 261173, 069345 |
| 63285 | 47010240 | 47011547 | 261173 |
| 63286 | 47071205 | 47072489 | |
| 63287 | 47493838 | 47496307 | 102893, 129636 |
| 63288 | 47533427 | 47533962 | 102893 |
| 63289 | 47667900 | 47668401 | 102893 |
| 63290 | 48192208 | 48192736 | |
| 63291 | 48198559 | 48200516 | 261538 |
| 63292 | 48237871 | 48241548 | 121270 |
| 63293 | 48268130 | 48270561 | 121270 |
| 63294 | 48277006 | 48279830 | 102910, 121270 |
| 63295 | 48309964 | 48310455 | 102910 |
| 63296 | 48333377 | 48335307 | 102910 |
| 63297 | 48399561 | 48400856 | 196470 |
| 63298 | 48416754 | 48420290 | 196470 |
| 63299 | 48439132 | 48440418 | 196470 |
| 63300 | 48443435 | 48444658 | 196470 |
| 63301 | 48468747 | 48469318 | 196470 |
| 63302 | 48513829 | 48514388 | |
| 63303 | 48526042 | 48526488 | 239013 |
| 63304 | 48533141 | 48534394 | |
| 63305 | 48558283 | 48559093 | |
| 63306 | 48559750 | 48561308 | |
| 63307 | 48593428 | 48595065 | 261267, 102921 |
| 63308 | 48595979 | 48596476 | 261267, 102921 |
| 63309 | 48625851 | 48626345 | 102921 |
| 63310 | 48642016 | 48644906 | 102921 |
| 63311 | 48657008 | 48658147 | 260086 |
| 63312 | 49682684 | 49684103 | 102935 |
| 63313 | 49889030 | 49892310 | 102935 |
| 63314 | 49917006 | 49918828 | |
| 63315 | 50058472 | 50060021 | 205423 |
| 63316 | 50069271 | 50069960 | 205423 |
| 63317 | 50099668 | 50100920 | 155393, 259843 |
| 63318 | 50186079 | 50189559 | 121274 |
| 63319 | 50218499 | 50220041 | 121274 |
| 63320 | 50227536 | 50228206 | 121274 |
| 63321 | 50236557 | 50237784 | 121274 |
| 63322 | 50278629 | 50280991 | 121281 |
| 63323 | 50401747 | 50403409 | 261393, 166164 |
| 63324 | 50564279 | 50564996 | |
| 63325 | 50581096 | 50582844 | 140807 |
| 63326 | 50625565 | 50626556 | 140807 |
| 63327 | 50650714 | 50651755 | 140807 |
| 63328 | 50698753 | 50699729 | |
| 63329 | 50729437 | 50732569 | 167207 |
| 63330 | 50732817 | 50734854 | 167207 |
| 63331 | 50774084 | 50780011 | 083799, 264947, 260929, |
| | | | 261644 |
| 63332 | 50839891 | 50840903 | 261703 |
| 63333 | 50876259 | 50877475 | |
| 63334 | 50877869 | 50879076 | |
| 63335 | 50913695 | 50914448 | 263082 |
| 63336 | 50958465 | 50961052 | |
| 63337 | 51091562 | 51092754 | |
| 63338 | 51128472 | 51130967 | |
| 63339 | 51141509 | 51142067 | 261282 |
| 63340 | 51145810 | 51147027 | |
| 63341 | 51153377 | 51155284 | |
| 63342 | 51181693 | 51192261 | 261238, 103449 |
| 63343 | 51244923 | 51245870 | |
| 63344 | 51277431 | 51278890 | 261047 |
| 63345 | 51393195 | 51394020 | |
| 63346 | 51415844 | 51416979 | |
| 63347 | 51435459 | 51440082 | |
| 63348 | 51440433 | 51444491 | |
| 63349 | 51464912 | 51466523 | |
| 63350 | 51479030 | 51479701 | |
| 63351 | 51556658 | 51559739 | |
| 63352 | 52013188 | 52014505 | |
| 63353 | 52028408 | 52028823 | |
| 63354 | 52084042 | 52084921 | 261190 |
| 63355 | 52225361 | 52226192 | |
| 63356 | 52227254 | 52227858 | |
| 63357 | 52330481 | 52331632 | |
| 63358 | 52380607 | 52381124 | |
| 63359 | 52417387 | 52418503 | |
| 63360 | 52441229 | 52444441 | |
| 63361 | 52522600 | 52523234 | 103460 |
| 63362 | 52526454 | 52527425 | 103460 |
| 63363 | 52530084 | 52530827 | 103460 |
| 63364 | 52578589 | 52581351 | 103460 |
| 63365 | 52586461 | 52587477 | 249231 |
| 63366 | 52598739 | 52599462 | 249231 |
| 63367 | 52611932 | 52612856 | 249231 |
| 63368 | 52644988 | 52647131 | 260963, 249231 |
| 63369 | 52776472 | 52777049 | |
| 63370 | 52846268 | 52847514 | |
| 63371 | 53081916 | 53083179 | 261550 |
| 63372 | 53085600 | 53091010 | 177200, 261550 |
| 63373 | 53095700 | 53096313 | 177200 |
| 63374 | 53114271 | 53115543 | 177200 |
| 63375 | 53118847 | 53119920 | 177200 |
| 63376 | 53123220 | 53126238 | 177200 |
| 63377 | 53127052 | 53128591 | 177200 |
| 63378 | 53132348 | 53135132 | 177200 |
| 63379 | 53145264 | 53146010 | 177200 |
| 63380 | 53163130 | 53166748 | 177200 |
| 63381 | 53467917 | 53470521 | 103479 |
| 63382 | 53532668 | 53533365 | 166971 |
| 63383 | 53536134 | 53539163 | 166971 |
| 63384 | 53543464 | 53546481 | |
| 63385 | 53737386 | 53738366 | 140718, 103494 |
| 63386 | 53795792 | 53796513 | 140718 |
| 63387 | 53809433 | 53810650 | 140718 |
| 63388 | 53814996 | 53815723 | 140718 |
| 63389 | 53855408 | 53857118 | 140718 |
| 63390 | 54208923 | 54211075 | |
| 63391 | 54214365 | 54215842 | |
| 63392 | 54300194 | 54300889 | 259517 |
| 63393 | 54454997 | 54455430 | |
| 63394 | 54477578 | 54478272 | |
| 63395 | 54662695 | 54663308 | |
| 63396 | 54691155 | 54692178 | |
| 63397 | 55656910 | 55657497 | |
| 63398 | 55670662 | 55671366 | |
| 63399 | 55794066 | 55794919 | 228695 |
| 63400 | 55866084 | 55867688 | 198848 |
| 63401 | 55869128 | 55870860 | |
| 63402 | 55905206 | 55906302 | 159398 |
| 63403 | 55983279 | 55983796 | 159398 |
| 63404 | 56223980 | 56226595 | 087258, 261439, 246379 |
| 63405 | 56228013 | 56228792 | 261439, 087258 |
| 63406 | 56244775 | 56247387 | 087258 |
| 63407 | 56267303 | 56268843 | 087258 |
| 63408 | 56279098 | 56280841 | 265281, 087258 |
| 63409 | 56297099 | 56298640 | 087258 |
| 63410 | 56303047 | 56304695 | 087258 |
| 63411 | 56328677 | 56329522 | 087258 |
| 63412 | 56390018 | 56392055 | 087258 |
| 63413 | 56404507 | 56405216 | 159461 |
| 63414 | 56410364 | 56410955 | 159461 |
| 63415 | 56416216 | 56418035 | 159461 |
| 63416 | 56441402 | 56444033 | 260621, 159461 |
| 63417 | 56445496 | 56446042 | 260621, 159461 |
| 63418 | 56458178 | 56459893 | 159461 |
| 63419 | 56484547 | 56486728 | 087263, 167005 |
| 63420 | 56553209 | 56554146 | 125124 |
| 63421 | 56573991 | 56574468 | |
| 63422 | 56585615 | 56586193 | |
| 63423 | 56638714 | 56649675 | 260823, 125148 |
| 63424 | 56658351 | 56661605 | 169715 |
| 63425 | 56665850 | 56667317 | 205364, 268317 |
| 63426 | 56671681 | 56672761 | 205362 |
| 63427 | 56691174 | 56692468 | 198417 |
| 63428 | 56701207 | 56705215 | 205358, 125144 |
| 63429 | 56712894 | 56714214 | |
| 63430 | 56715046 | 56717916 | 259827, 187193 |
| 63431 | 56718118 | 56719550 | 259827 |
| 63432 | 56763556 | 56764928 | 102900, 261302 |
| 63433 | 56834106 | 56835306 | 102900 |
| 63434 | 56890476 | 56891443 | |
| 63435 | 56919630 | 56920213 | 070915 |
| 63436 | 56964821 | 56967532 | 051108 |
| 63437 | 56979712 | 56980738 | |
| 63438 | 56998511 | 56999253 | 087237 |
| 63439 | 57001656 | 57002022 | 087237 |
| 63440 | 57022530 | 57024491 | 140853 |
| 63441 | 57033086 | 57035420 | 140853 |
| 63442 | 57094810 | 57096138 | 140853 |
| 63443 | 57118017 | 57118822 | |
| 63444 | 57125947 | 57127550 | 140848, 260148 |
| 63445 | 57212714 | 57213840 | 172775 |
| 63446 | 57218639 | 57222181 | 159579, 172775 |
| 63447 | 57226692 | 57229707 | 159579 |
| 63448 | 57272449 | 57273507 | 159579 |
| 63449 | 57278492 | 57280093 | 102931 |
| 63450 | 57285346 | 57287770 | 102931, 260946 |
| 63451 | 57318249 | 57319108 | 102934 |
| 63452 | 57332889 | 57335276 | |
| 63453 | 57336646 | 57340296 | |
| 63454 | 57347732 | 57349590 | |
| 63455 | 57349900 | 57351698 | |
| 63456 | 57405619 | 57408581 | 006210 |
| 63457 | 57413261 | 57414218 | 006210 |
| 63458 | 57450497 | 57452280 | |
| 63459 | 57480337 | 57482220 | 088682, 005194 |
| 63460 | 57496192 | 57497546 | 102978, 260345 |
| 63461 | 57503904 | 57507239 | 125170, 102978 |
| 63462 | 57513352 | 57515035 | 125170 |
| 63463 | 57519279 | 57521162 | 125170 |
| 63464 | 57534511 | 57535247 | |
| 63465 | 57569622 | 57571009 | 135736 |
| 63466 | 57624535 | 57625720 | 159618 |
| 63467 | 57640089 | 57641408 | |
| 63468 | 57661330 | 57663092 | 261633, 205336 |
| 63469 | 57700400 | 57702511 | 182885 |
| 63470 | 57711265 | 57712018 | 182885 |
| 63471 | 57768191 | 57771174 | 140854 |
| 63472 | 57815204 | 57817188 | 140859 |
| 63473 | 57830550 | 57832769 | 187185, 140859 |
| 63474 | 57834660 | 57836685 | 140859, 187185 |
| 63475 | 57840936 | 57842367 | 140859, 187185 |
| 63476 | 57843086 | 57845347 | 140859, 187185 |
| 63477 | 57858821 | 57859618 | 140859 |
| 63478 | 57859636 | 57862195 | 140859 |
| 63479 | 57863405 | 57864139 | 140859 |
| 63480 | 57864678 | 57865207 | 140859 |
| 63481 | 57896001 | 57898294 | 140859 |
| 63482 | 57904495 | 57906740 | |
| 63483 | 57922962 | 57925485 | 070729 |
| 63484 | 57925741 | 57930265 | 070729 |
| 63485 | 58033474 | 58035993 | 166188, 103005 |
| 63486 | 58056071 | 58057264 | |
| 63487 | 58058063 | 58061792 | 102996 |
| 63488 | 58064316 | 58065910 | 102996 |
| 63489 | 58066524 | 58067421 | 102996 |
| 63490 | 58154834 | 58156198 | 070761 |
| 63491 | 58161836 | 58164114 | 260927, 070761 |
| 63492 | 58230882 | 58231851 | 260867, 070770 |
| 63493 | 58231936 | 58232482 | 260867 |
| 63494 | 58255911 | 58257091 | |
| 63495 | 58283665 | 58284222 | 103021 |
| 63496 | 58362855 | 58363444 | 181938 |
| 63497 | 58370470 | 58371288 | 181938 |
| 63498 | 58426078 | 58426819 | 181938 |
| 63499 | 58492269 | 58495363 | 260186 |
| 63500 | 58548519 | 58550291 | 103037 |
| 63501 | 58638114 | 58638530 | 125107 |
| 63502 | 58662397 | 58664761 | 125107 |
| 63503 | 58667960 | 58668971 | |
| 63504 | 58717201 | 58719409 | 103042 |
| 63505 | 58754674 | 58755519 | 125166 |
| 63506 | 58764579 | 58765109 | 125166 |
| 63507 | 58766416 | 58768597 | 245768, 125166 |
| 63508 | 58881683 | 58882564 | 261638, 245768 |
| 63509 | 59244719 | 59245422 | |
| 63510 | 61629340 | 61630091 | |
| 63511 | 63370879 | 63371639 | 260658 |
| 63512 | 64194882 | 64195548 | |
| 63513 | 64216814 | 64217571 | |
| 63514 | 65104574 | 65105328 | 140937 |
| 63515 | 65910533 | 65911017 | |
| 63516 | 65930434 | 65931284 | |
| 63517 | 65943258 | 65943714 | |
| 63518 | 65956775 | 65957360 | |
| 63519 | 65979174 | 65980050 | |
| 63520 | 65999425 | 65999975 | |
| 63521 | 66231726 | 66232325 | |
| 63522 | 66304218 | 66304813 | |
| 63523 | 66535995 | 66537897 | 260851 |
| 63524 | 66543061 | 66548294 | 260755, 166548, 260851 |
| 63525 | 66549002 | 66552569 | 260851, 166548 |
| 63526 | 66582696 | 66584564 | 261519, 166548 |
| 63527 | 66585501 | 66586850 | 217555, 254788, 166548 |
| 63528 | 66729571 | 66730931 | 183723 |
| 63529 | 66774075 | 66776396 | 135720 |
| 63530 | 66784386 | 66786353 | 246777, 135720 |
| 63531 | 66864061 | 66865332 | 159593 |
| 63532 | 66913699 | 66915332 | 172840 |
| 63533 | 66932702 | 66935022 | |
| 63534 | 66958758 | 66963140 | 166592 |
| 63535 | 66963831 | 66965144 | |
| 63536 | 66967028 | 66971473 | 172831, 166595 |
| 63537 | 66995708 | 66996510 | 172828, 265408 |
| 63538 | 66997743 | 66998439 | 172828 |
| 63539 | 67029782 | 67031682 | 172824 |
| 63540 | 67056464 | 67057225 | |
| 63541 | 67062171 | 67064347 | 067955 |
| 63542 | 67142909 | 67145557 | 125149 |
| 63543 | 67147843 | 67148358 | 125149 |
| 63544 | 67193010 | 67195071 | 135722, 102871 |
| 63545 | 67198398 | 67198934 | 135722, 102878, 265690 |
| 63546 | 67203524 | 67205505 | 140939, 102878 |
| 63547 | 67207394 | 67209063 | 140939 |
| 63548 | 67217373 | 67218389 | 179044, 196123 |
| 63549 | 67225710 | 67227238 | 205250 |
| 63550 | 67232831 | 67233289 | 102890 |
| 63551 | 67260131 | 67261872 | 168701, 237102, 125122 |
| 63552 | 67449645 | 67450694 | 159714 |
| 63553 | 67464090 | 67466072 | 176387, 261320 |
| 63554 | 67477009 | 67477611 | 159720 |
| 63555 | 67479280 | 67480529 | 159720 |
| 63556 | 67504165 | 67504638 | 159720 |
| 63557 | 67513952 | 67515881 | 270049, 159720 |
| 63558 | 67554892 | 67555799 | 039523 |
| 63559 | 67561321 | 67565033 | 039523 |
| 63560 | 67571047 | 67572664 | 261396, 039523 |
| 63561 | 67580582 | 67581341 | 039523 |
| 63562 | 67595247 | 67598140 | 259804, 102974, 261386 |
| 63563 | 67675551 | 67676587 | |
| 63564 | 67693404 | 67694967 | 102981, 102977 |
| 63565 | 67699859 | 67702015 | 159761, 124074 |
| 63566 | 67706178 | 67709123 | 141098 |
| 63567 | 67751967 | 67755185 | 141098 |
| 63568 | 67758924 | 67761568 | 141084 |
| 63569 | 67803493 | 67808812 | 141084 |
| 63570 | 67813059 | 67815345 | 141084 |
| 63571 | 67838577 | 67841314 | 102904, 141084 |
| 63572 | 67866961 | 67868104 | 102901 |
| 63573 | 67875247 | 67877281 | 168286, 102901 |
| 63574 | 67879869 | 67881870 | 102898, 102901 |
| 63575 | 67891436 | 67892140 | 102898 |
| 63576 | 67906477 | 67907820 | 038358 |
| 63577 | 67926655 | 67928761 | 159792 |
| 63578 | 67949840 | 67952695 | 159792 |
| 63579 | 67968855 | 67971586 | 261884, 205220 |
| 63580 | 67976728 | 67977661 | 124067, 267660, 213398 |
| 63581 | 67987936 | 67988823 | 124067 |
| 63582 | 67995293 | 67996054 | 124067 |
| 63583 | 67999270 | 68001116 | 124067 |
| 63584 | 68001213 | 68003430 | 124067 |
| 63585 | 68026974 | 68028045 | 167261, 167264 |
| 63586 | 68055707 | 68058207 | 182810, 167264 |
| 63587 | 68113425 | 68114317 | 261864 |
| 63588 | 68118037 | 68120900 | 072736, 261864 |
| 63589 | 68125376 | 68126247 | 072736, 261864 |
| 63590 | 68130811 | 68131567 | 072736, 261864 |
| 63591 | 68267916 | 68272482 | 261469, 103067 |
| 63592 | 68273577 | 68274162 | |
| 63593 | 68278309 | 68279784 | 103066 |
| 63594 | 68297440 | 68300120 | 103064 |
| 63595 | 68343537 | 68345975 | 132600, 103061 |
| 63596 | 68365764 | 68366783 | 132600 |
| 63597 | 68430148 | 68431037 | 103056 |
| 63598 | 68440352 | 68441229 | 103056 |
| 63599 | 68442117 | 68442869 | 103056 |
| 63600 | 68460124 | 68460791 | 103056 |
| 63601 | 68482040 | 68482880 | 103056 |
| 63602 | 68503486 | 68504601 | |
| 63603 | 68507367 | 68508034 | |
| 63604 | 68515469 | 68515991 | |
| 63605 | 68554451 | 68555052 | |
| 63606 | 68738115 | 68738706 | 062038 |
| 63607 | 68766005 | 68766939 | |
| 63608 | 68771092 | 68772889 | 039068 |
| 63609 | 68773758 | 68777444 | 200558, 039068 |
| 63610 | 68778004 | 68779314 | 039068 |
| 63611 | 68789234 | 68791088 | 039068 |
| 63612 | 68794149 | 68796293 | 039068 |
| 63613 | 68812190 | 68814208 | 039068 |
| 63614 | 68820371 | 68821317 | 039068 |
| 63615 | 68823978 | 68825409 | 039068 |
| 63616 | 68826060 | 68827961 | 039068 |
| 63617 | 68877135 | 68878706 | 103047 |
| 63618 | 68907486 | 68908350 | 103047 |
| 63619 | 68926002 | 68926516 | 103047 |
| 63620 | 68933861 | 68934355 | 103047 |
| 63621 | 68935858 | 68937203 | 103047 |
| 63622 | 68942801 | 68943597 | 103047 |
| 63623 | 68959532 | 68960143 | 103047 |
| 63624 | 68970541 | 68971365 | 103047, 266801, 260999 |
| 63625 | 68982183 | 68982815 | 103047 |
| 63626 | 69025624 | 69029625 | 103047 |
| 63627 | 69050828 | 69051802 | 103047 |
| 63628 | 69065632 | 69066247 | 103047 |
| 63629 | 69073572 | 69075309 | 103047 |
| 63630 | 69090806 | 69092968 | 103047 |
| 63631 | 69107291 | 69107655 | 103047 |
| 63632 | 69122914 | 69123938 | |
| 63633 | 69139094 | 69140793 | 103044 |
| 63634 | 69165402 | 69167391 | 168802, 141076 |
| 63635 | 69217619 | 69218116 | 141076 |
| 63636 | 69220368 | 69222364 | 168807, 141076 |
| 63637 | 69251277 | 69252166 | 141076, 168807 |
| 63638 | 69255876 | 69256566 | 141076, 168807 |
| 63639 | 69332608 | 69333126 | 168807 |
| 63640 | 69344577 | 69346541 | 132612, 260914 |
| 63641 | 69349183 | 69350910 | 132612, 260914 |
| 63642 | 69359847 | 69361027 | 213380 |
| 63643 | 69362662 | 69365342 | 258429, 213380, 272617 |
| 63644 | 69372685 | 69374816 | 132603, 213380, 260371, |
| | | | 259900 |
| 63645 | 69385066 | 69385973 | 259900, 157315, 260371 |
| 63646 | 69418703 | 69420199 | 132604 |
| 63647 | 69429790 | 69431063 | 132604 |
| 63648 | 69457968 | 69459326 | 103018 |
| 63649 | 69482411 | 69483310 | 103018 |
| 63650 | 69564419 | 69565068 | |
| 63651 | 69595763 | 69596251 | |
| 63652 | 69598694 | 69601665 | 102908, 223109 |
| 63653 | 69787978 | 69789266 | 141101 |
| 63654 | 69795501 | 69797417 | 198373 |
| 63655 | 69856075 | 69856648 | 198373 |
| 63656 | 69864603 | 69865391 | 198373 |
| 63657 | 69892619 | 69893366 | 198373 |
| 63658 | 69975358 | 69976159 | 198373 |
| 63659 | 70185716 | 70187472 | 090857 |
| 63660 | 70284809 | 70286189 | 223496 |
| 63661 | 70322138 | 70324425 | 157349, 090861 |
| 63662 | 70332465 | 70334136 | 260537, 157349 |
| 63663 | 70379744 | 70381862 | 168872, 261777, 260537 |
| 63664 | 70413825 | 70416261 | 260111, 157350 |
| 63665 | 70444462 | 70445010 | 157350 |
| 63666 | 70461291 | 70463543 | 157350 |
| 63667 | 70472013 | 70473946 | 157350 |
| 63668 | 70487836 | 70489244 | 157353 |
| 63669 | 70546035 | 70547386 | 103051 |
| 63670 | 70556761 | 70558632 | 189091, 103051 |
| 63671 | 70715389 | 70717817 | 132613 |
| 63672 | 70718613 | 70720323 | 132613 |
| 63673 | 70727325 | 70728960 | 103043 |
| 63674 | 70729146 | 70730047 | 103043 |
| 63675 | 70735602 | 70736418 | 103043 |
| 63676 | 70743043 | 70744218 | 103043 |
| 63677 | 70745130 | 70745703 | 103043 |
| 63678 | 70748318 | 70750789 | 103043 |
| 63679 | 70757860 | 70760583 | 103043 |
| 63680 | 70771379 | 70772935 | 103043 |
| 63681 | 70779205 | 70783512 | 260156, 103043 |
| 63682 | 70784904 | 70786255 | 103043 |
| 63683 | 70793983 | 70795290 | 103043, 214353 |
| 63684 | 70834220 | 70835377 | 103043 |
| 63685 | 71322203 | 71324090 | 180917 |
| 63686 | 71371914 | 71372829 | |
| 63687 | 71385064 | 71385737 | |
| 63688 | 71460217 | 71461189 | 261348 |
| 63689 | 71495032 | 71496804 | 247324, 261611, 167377 |
| 63690 | 71507765 | 71510027 | 261611, 157429 |
| 63691 | 71517873 | 71518825 | 261611, 157429 |
| 63692 | 71548795 | 71549510 | 260520, 157429 |
| 63693 | 71589298 | 71589856 | 157429 |
| 63694 | 71591995 | 71592618 | 157429 |
| 63695 | 71597742 | 71601044 | 260886, 198650, 157429 |
| 63696 | 71605188 | 71608192 | 260886, 198650 |
| 63697 | 71609105 | 71611339 | 198650, 260886 |
| 63698 | 71614927 | 71616598 | |
| 63699 | 71618571 | 71620188 | |
| 63700 | 71623985 | 71629036 | |
| 63701 | 71634415 | 71635419 | |
| 63702 | 71642049 | 71642703 | |
| 63703 | 71659775 | 71660787 | 140832, 260593 |
| 63704 | 71700201 | 71701786 | 040199 |
| 63705 | 71743023 | 71743928 | 040199 |
| 63706 | 71756548 | 71758333 | 261513, 040199 |
| 63707 | 71760542 | 71761393 | 260612 |
| 63708 | 71815562 | 71816150 | 166747 |
| 63709 | 71841385 | 71844260 | 166747 |
| 63710 | 71878896 | 71880938 | 224470, 182149 |
| 63711 | 71892558 | 71893112 | 224470, 182149 |
| 63712 | 71917308 | 71918801 | 224470, 102984, 182149 |
| 63713 | 71927937 | 71930509 | 102984, 182149 |
| 63714 | 71961219 | 71961693 | 182149 |
| 63715 | 72042054 | 72043495 | 102967 |
| 63716 | 72050811 | 72051460 | 102967 |
| 63717 | 72057112 | 72059274 | 102967 |
| 63718 | 72061535 | 72062443 | |
| 63719 | 72075769 | 72077449 | |
| 63720 | 72084703 | 72085473 | 140830 |
| 63721 | 72087592 | 72088547 | 257017, 261701, 140830 |
| 63722 | 72126642 | 72128605 | 140829, 140830 |
| 63723 | 72845042 | 72845685 | 140836 |
| 63724 | 72870063 | 72870747 | 140836 |
| 63725 | 72881559 | 72883883 | 140836 |
| 63726 | 72910405 | 72912861 | 140836 |
| 63727 | 72979898 | 72982733 | 140836 |
| 63728 | 73008160 | 73008748 | 140836 |
| 63729 | 73012079 | 73012609 | 140836 |
| 63730 | 73049460 | 73050248 | 140836 |
| 63731 | 73053354 | 73055538 | 140836 |
| 63732 | 73061473 | 73062270 | 140836 |
| 63733 | 73081113 | 73083171 | 140836 |
| 63734 | 73086566 | 73089201 | 140836 |
| 63735 | 73089214 | 73093567 | 140836 |
| 63736 | 73103738 | 73105722 | |
| 63737 | 73236080 | 73236954 | |
| 63738 | 73238391 | 73239463 | |
| 63739 | 73265673 | 73267084 | 261227 |
| 63740 | 73299220 | 73300117 | |
| 63741 | 73385053 | 73385458 | |
| 63742 | 74107327 | 74107795 | 261404 |
| 63743 | 74141591 | 74142174 | 261404 |
| 63744 | 74187249 | 74188188 | 261404 |
| 63745 | 74324967 | 74326718 | 261404 |
| 63746 | 74329365 | 74331796 | 103035, 261404 |
| 63747 | 74604198 | 74606310 | 090863 |
| 63748 | 74639765 | 74641843 | 090863 |
| 63749 | 74660902 | 74662935 | 168411 |
| 63750 | 74669285 | 74669630 | 168411 |
| 63751 | 74700070 | 74701274 | 168411 |
| 63752 | 74733785 | 74735504 | 168404 |
| 63753 | 74743195 | 74743817 | |
| 63754 | 74776827 | 74778713 | 103089 |
| 63755 | 74957759 | 74958339 | 103091 |
| 63756 | 75018090 | 75019388 | 103091 |
| 63757 | 75025312 | 75026721 | 103091 |
| 63758 | 75031895 | 75034124 | 186187, 103091 |
| 63759 | 75045043 | 75046405 | 186187 |
| 63760 | 75082369 | 75083115 | 186187 |
| 63761 | 75087540 | 75088638 | 186187 |
| 63762 | 75103814 | 75104726 | 186187 |
| 63763 | 75142264 | 75145721 | 247033, 186187 |
| 63764 | 75149665 | 75150945 | 166816 |
| 63765 | 75182206 | 75183206 | 184517 |
| 63766 | 75267521 | 75268715 | 050820 |
| 63767 | 75277964 | 75280704 | 050820 |
| 63768 | 75283866 | 75286002 | 050820 |
| 63769 | 75295835 | 75300822 | 050820 |
| 63770 | 75301624 | 75302258 | 050820 |
| 63771 | 75322965 | 75323513 | |
| 63772 | 75338209 | 75341344 | 153774 |
| 63773 | 75351663 | 75352432 | 153774 |
| 63774 | 75385122 | 75385666 | 153774 |
| 63775 | 75410308 | 75411015 | 153774 |
| 63776 | 75466460 | 75468474 | 153774, 261717 |
| 63777 | 75497647 | 75499058 | 261717, 166822 |
| 63778 | 75550415 | 75551612 | |
| 63779 | 75561752 | 75562292 | |
| 63780 | 75584081 | 75584689 | 259992, 205084 |
| 63781 | 75599728 | 75601106 | 034713, 259992 |
| 63782 | 75656067 | 75657543 | 065457 |
| 63783 | 75680698 | 75683500 | 166848, 065427 |
| 63784 | 76255781 | 76256816 | |
| 63785 | 76462464 | 76463244 | 152910 |
| 63786 | 76562625 | 76567205 | 152910 |
| 63787 | 76608026 | 76609292 | 261833 |
| 63788 | 76913225 | 76913945 | 259995 |
| 63789 | 76931400 | 76932576 | 259995 |
| 63790 | 76933243 | 76936157 | 259995 |
| 63791 | 76944761 | 76945366 | 259995 |
| 63792 | 76959853 | 76960915 | 259995 |
| 63793 | 76980618 | 76981660 | |
| 63794 | 76998167 | 76998996 | |
| 63795 | 77003724 | 77005072 | |
| 63796 | 77223773 | 77226075 | 103111 |
| 63797 | 77745412 | 77746450 | |
| 63798 | 77755817 | 77757581 | 140876 |
| 63799 | 77783308 | 77783797 | |
| 63800 | 77797652 | 77798845 | |
| 63801 | 77808773 | 77812650 | |
| 63802 | 77813695 | 77815734 | |
| 63803 | 78132898 | 78134126 | 186153 |
| 63804 | 78274391 | 78274911 | 260969, 186153 |
| 63805 | 78276079 | 78276597 | 186153 |
| 63806 | 78286255 | 78287134 | 186153 |
| 63807 | 78318386 | 78319433 | 186153 |
| 63808 | 78326629 | 78327570 | 186153 |
| 63809 | 78477183 | 78478312 | 186153 |
| 63810 | 78517119 | 78518077 | 186153 |
| 63811 | 78539165 | 78540791 | 260733, 186153 |
| 63812 | 78630762 | 78631424 | 186153 |
| 63813 | 78717628 | 78718621 | 186153 |
| 63814 | 78739528 | 78741029 | 186153 |
| 63815 | 78850505 | 78851313 | 186153 |
| 63816 | 79021314 | 79021901 | 186153 |
| 63817 | 79071494 | 79072625 | 186153 |
| 63818 | 79286367 | 79287548 | |
| 63819 | 79328384 | 79328928 | |
| 63820 | 79575864 | 79576599 | |
| 63821 | 79598197 | 79599228 | |
| 63822 | 79631468 | 79633038 | 178573 |
| 63823 | 79633288 | 79637102 | 178573 |
| 63824 | 79757117 | 79758242 | 260876, 261390 |
| 63825 | 79803992 | 79804871 | 261390, 260876 |
| 63826 | 79931132 | 79932342 | |
| 63827 | 80004264 | 80004867 | |
| 63828 | 80057534 | 80058510 | |
| 63829 | 80060810 | 80061570 | |
| 63830 | 80207389 | 80209327 | 259867 |
| 63831 | 80231483 | 80231977 | 259867 |
| 63832 | 80299427 | 80300161 | 259867 |
| 63833 | 80334207 | 80335211 | 259867 |
| 63834 | 80409786 | 80410348 | 259867 |
| 63835 | 80438749 | 80439227 | 259867 |
| 63836 | 80441090 | 80441778 | 259867 |
| 63837 | 80493096 | 80493735 | 259867 |
| 63838 | 80574036 | 80575158 | 168589, 259867 |
| 63839 | 80577871 | 80578643 | 168589, 259867 |
| 63840 | 80751493 | 80752197 | 166446 |
| 63841 | 80793043 | 80794190 | 166446 |
| 63842 | 80837094 | 80837923 | 166446 |
| 63843 | 80837931 | 80838318 | 166446 |
| 63844 | 80965395 | 80967074 | |
| 63845 | 81014642 | 81015189 | 103121 |
| 63846 | 81026040 | 81027486 | 103121 |
| 63847 | 81030699 | 81031603 | 103121 |
| 63848 | 81039693 | 81042002 | 166451, 103121 |
| 63849 | 81068906 | 81070801 | 166454, 260213 |
| 63850 | 81110264 | 81111494 | 245059, 166455, 260643 |
| 63851 | 81129065 | 81130659 | 140905 |
| 63852 | 81156559 | 81157683 | 166473 |
| 63853 | 81207969 | 81209783 | 166473 |
| 63854 | 81232057 | 81232714 | 166473 |
| 63855 | 81271806 | 81272689 | 135697 |
| 63856 | 81348076 | 81349815 | 261609 |
| 63857 | 81434575 | 81435408 | |
| 63858 | 81438878 | 81439908 | |
| 63859 | 81477388 | 81482637 | 153815 |
| 63860 | 81485355 | 81486550 | 153815 |
| 63861 | 81489080 | 81492542 | 153815 |
| 63862 | 81496263 | 81496772 | 153815 |
| 63863 | 81498544 | 81501231 | 153815 |
| 63864 | 81506734 | 81508056 | 153815 |
| 63865 | 81512447 | 81512961 | 153815 |
| 63866 | 81525098 | 81528561 | 153815 |
| 63867 | 81546511 | 81547949 | 153815 |
| 63868 | 81556949 | 81557552 | 153815 |
| 63869 | 81566167 | 81566984 | 153815 |
| 63870 | 81567459 | 81568117 | 153815 |
| 63871 | 81582483 | 81583744 | 153815 |
| 63872 | 81584511 | 81587694 | 153815 |
| 63873 | 81601524 | 81602534 | 153815 |
| 63874 | 81616177 | 81617275 | 153815 |
| 63875 | 81658512 | 81659920 | 153815 |
| 63876 | 81670000 | 81670347 | 153815 |
| 63877 | 81763696 | 81764231 | |
| 63878 | 81812200 | 81813728 | 197943 |
| 63879 | 81907368 | 81908059 | 197943 |
| 63880 | 81921990 | 81924406 | 197943 |
| 63881 | 81990012 | 81990599 | 197943 |
| 63882 | 82030966 | 82031773 | 184860 |
| 63883 | 82044249 | 82045720 | 184860 |
| 63884 | 82060319 | 82061888 | |
| 63885 | 82064574 | 82066030 | |
| 63886 | 82066267 | 82070085 | 086696 |
| 63887 | 82077325 | 82078555 | 261235, 086696 |
| 63888 | 82084175 | 82084992 | 086696, 261235 |
| 63889 | 82088912 | 82090569 | 086696, 261235 |
| 63890 | 82093368 | 82094306 | 086696, 261235 |
| 63891 | 82095187 | 82098259 | 086696, 261235 |
| 63892 | 82115542 | 82118196 | 086696, 261235 |
| 63893 | 82121931 | 82122645 | 086696, 261235 |
| 63894 | 82169417 | 82169920 | 261235 |
| 63895 | 82203135 | 82204772 | 261029, 135698 |
| 63896 | 82685911 | 82689467 | 140945 |
| 63897 | 82794787 | 82796026 | 140945 |
| 63898 | 83170861 | 83171576 | 140945 |
| 63899 | 83841350 | 83842681 | 230989, 260228 |
| 63900 | 83859565 | 83860248 | |
| 63901 | 83932066 | 83933947 | 103150 |
| 63902 | 83960156 | 83961697 | 260300 |
| 63903 | 83976446 | 83977282 | 260300 |
| 63904 | 83979877 | 83981965 | 140961, 260300 |
| 63905 | 83985909 | 83989491 | 140961 |
| 63906 | 83990095 | 83990891 | 140961 |
| 63907 | 84149280 | 84151585 | 260018, 140943 |
| 63908 | 84177829 | 84179096 | 154099, 103160 |
| 63909 | 84208580 | 84212910 | 103168, 154099 |
| 63910 | 84219912 | 84221068 | 103168 |
| 63911 | 84537136 | 84539179 | 140950 |
| 63912 | 84676550 | 84677120 | |
| 63913 | 84681519 | 84683449 | 135686 |
| 63914 | 84732788 | 84734869 | 103194 |
| 63915 | 84763122 | 84764439 | 103194 |
| 63916 | 84773183 | 84775754 | 103194 |
| 63917 | 84865994 | 84867157 | 103196 |
| 63918 | 84884108 | 84886509 | 103196 |
| 63919 | 84927170 | 84928269 | 103196 |
| 63920 | 84946817 | 84947701 | 103196 |
| 63921 | 84966804 | 84968487 | 260340 |
| 63922 | 85027263 | 85029472 | 153786 |
| 63923 | 85031722 | 85035181 | 153786 |
| 63924 | 85037768 | 85038495 | 153786 |
| 63925 | 85044022 | 85046157 | 153786 |
| 63926 | 85060361 | 85062547 | 135709 |
| 63927 | 85148436 | 85149269 | |
| 63928 | 85182460 | 85183878 | |
| 63929 | 85185331 | 85185842 | |
| 63930 | 85192732 | 85194590 | |
| 63931 | 85200240 | 85202168 | |
| 63932 | 85205743 | 85208816 | 262601 |
| 63933 | 85215302 | 85215873 | 262601 |
| 63934 | 85233037 | 85235152 | |
| 63935 | 85246902 | 85247524 | |
| 63936 | 85253447 | 85254485 | |
| 63937 | 85319120 | 85321728 | 179219 |
| 63938 | 85322423 | 85323178 | |
| 63939 | 85339685 | 85340151 | 266307 |
| 63940 | 85341106 | 85341738 | |
| 63941 | 85355084 | 85356390 | |
| 63942 | 85368100 | 85369507 | |
| 63943 | 85372330 | 85373310 | |
| 63944 | 85384618 | 85384976 | |
| 63945 | 85392009 | 85396177 | 261567 |
| 63946 | 85409647 | 85410587 | |
| 63947 | 85423639 | 85424813 | |
| 63948 | 85449830 | 85450281 | |
| 63949 | 85455885 | 85456838 | |
| 63950 | 85482246 | 85483421 | |
| 63951 | 85494468 | 85495066 | |
| 63952 | 85499471 | 85499915 | |
| 63953 | 85525368 | 85526466 | |
| 63954 | 85547603 | 85550442 | |
| 63955 | 85556846 | 85559879 | |
| 63956 | 85560467 | 85561633 | |
| 63957 | 85568767 | 85570104 | |
| 63958 | 85571218 | 85572319 | |
| 63959 | 85573122 | 85573764 | |
| 63960 | 85586390 | 85592326 | |
| 63961 | 85596621 | 85598466 | |
| 63962 | 85643799 | 85649691 | 131149 |
| 63963 | 85669424 | 85670344 | 131149 |
| 63964 | 85675040 | 85676077 | 131149 |
| 63965 | 85684789 | 85685498 | 131149 |
| 63966 | 85707529 | 85708189 | 131149 |
| 63967 | 85714733 | 85715397 | 131153 |
| 63968 | 85751319 | 85752037 | 154102 |
| 63969 | 85753570 | 85754372 | 154102 |
| 63970 | 85815271 | 85816019 | 252311, 131148 |
| 63971 | 85831979 | 85834473 | 131143, 131148 |
| 63972 | 85854729 | 85855628 | |
| 63973 | 85881156 | 85882387 | |
| 63974 | 85883680 | 85884855 | |
| 63975 | 85921636 | 85923362 | |
| 63976 | 85924481 | 85925432 | |
| 63977 | 85932005 | 85933207 | 140968 |
| 63978 | 85935396 | 85935961 | 140968 |
| 63979 | 85949577 | 85950683 | 140968 |
| 63980 | 85973087 | 85974538 | |
| 63981 | 86007997 | 86009781 | |
| 63982 | 86553462 | 86554218 | 270020 |
| 63983 | 86588028 | 86589789 | 103248 |
| 63984 | 86697606 | 86699268 | 261161 |
| 63985 | 87202954 | 87203679 | 260456 |
| 63986 | 87350323 | 87352616 | 270006, 260456, 131152 |
| 63987 | 87372653 | 87373212 | 103264 |
| 63988 | 87408939 | 87409814 | 103264 |
| 63989 | 87412776 | 87413309 | 103264 |
| 63990 | 87415781 | 87418685 | 140941, 103264 |
| 63991 | 87421199 | 87423670 | 103264, 140941 |
| 63992 | 87424419 | 87427503 | 103264, 140941 |
| 63993 | 87489994 | 87491706 | 140948 |
| 63994 | 87494932 | 87495969 | 140948 |
| 63995 | 87497313 | 87499904 | 140948 |
| 63996 | 87517768 | 87518982 | 140948 |
| 63997 | 87526246 | 87527068 | 260750 |
| 63998 | 87575124 | 87575782 | |
| 63999 | 87588225 | 87589689 | |
| 64000 | 87668992 | 87670445 | 154118 |
| 64001 | 87798637 | 87800592 | 104731 |
| 64002 | 87811127 | 87813365 | 260498 |
| 64003 | 87818347 | 87818953 | 260177 |
| 64004 | 87822382 | 87825693 | 260177 |
| 64005 | 87870258 | 87877164 | 260466, 103257 |
| 64006 | 87878775 | 87880557 | 103257 |
| 64007 | 87880959 | 87881889 | 103257 |
| 64008 | 87884071 | 87886240 | 103257 |
| 64009 | 87886294 | 87889323 | 103257 |
| 64010 | 87981457 | 87986435 | 172530 |
| 64011 | 88016191 | 88016786 | 172530 |
| 64012 | 88454579 | 88455139 | |
| 64013 | 88519074 | 88519507 | |
| 64014 | 88519511 | 88523109 | 179588 |
| 64015 | 88539598 | 88541145 | 179588 |
| 64016 | 88553882 | 88555332 | 179588 |
| 64017 | 88564923 | 88565749 | 179588 |
| 64018 | 88566194 | 88567116 | 179588 |
| 64019 | 88570856 | 88572559 | 179588 |
| 64020 | 88635841 | 88638089 | 158545 |
| 64021 | 88684574 | 88685532 | 158545 |
| 64022 | 88728635 | 88730363 | 260630, 167508 |
| 64023 | 88731408 | 88731937 | 260630 |
| 64024 | 88752587 | 88753499 | 185669, 260630 |
| 64025 | 88771816 | 88773799 | 174177, 158717 |
| 64026 | 88825521 | 88826323 | 103335 |
| 64027 | 88829623 | 88830985 | 103335 |
| 64028 | 88831098 | 88832004 | 103335 |
| 64029 | 88848916 | 88852290 | 103335 |
| 64030 | 88853709 | 88854594 | |
| 64031 | 88869343 | 88870297 | 167513 |
| 64032 | 88876787 | 88879018 | 198931 |
| 64033 | 88922721 | 88924528 | 141012, 167515 |
| 64034 | 88946555 | 88948314 | 259881, 129993 |
| 64035 | 88949308 | 88949902 | 259881, 129993 |
| 64036 | 89159770 | 89161193 | 176715 |
| 64037 | 89184702 | 89185110 | 176715 |
| 64038 | 89282823 | 89284875 | 170100, 259877 |
| 64039 | 89298438 | 89298994 | |
| 64040 | 89307052 | 89308579 | |
| 64041 | 89313251 | 89314059 | |
| 64042 | 89322779 | 89324503 | 260637 |
| 64043 | 89385259 | 89387910 | 261253, 167522 |
| 64044 | 89404909 | 89405925 | 167522 |
| 64045 | 89411671 | 89412707 | 167522 |
| 64046 | 89430245 | 89431870 | 167522 |
| 64047 | 89461413 | 89462113 | 167522, 261574 |
| 64048 | 89477055 | 89477652 | 167522, 261574 |
| 64049 | 89484604 | 89485503 | 167522 |
| 64050 | 89508175 | 89510271 | 252887, 167522 |
| 64051 | 89540181 | 89540976 | 167522 |
| 64052 | 89544935 | 89545998 | 167522 |
| 64053 | 89555011 | 89558198 | 197912, 167522 |
| 64054 | 89568338 | 89569540 | 261118, 197912 |
| 64055 | 89571196 | 89573065 | 197912 |
| 64056 | 89573273 | 89575695 | 197912 |
| 64057 | 89625765 | 89628468 | 167526, 200084 |
| 64058 | 89631871 | 89632770 | |
| 64059 | 89648361 | 89649444 | 178773 |
| 64060 | 89723181 | 89725216 | 167523, 131165 |
| 64061 | 89752787 | 89754726 | 260259, 185324 |
| 64062 | 89766284 | 89768167 | 158792 |
| 64063 | 89772287 | 89772804 | |
| 64064 | 89786321 | 89789443 | 158805, 075399 |
| 64065 | 89830587 | 89832210 | 187741 |
| 64066 | 89883190 | 89883491 | |
| 64067 | 89893827 | 89895455 | 204991 |
| 64068 | 89903550 | 89904633 | 204991 |
| 64069 | 89939247 | 89940918 | 141002 |
| 64070 | 90014900 | 90015269 | 140995 |
| 64071 | 90037922 | 90040124 | 223959, 261317, 177946 |
| 64072 | 90062001 | 90063580 | 223959 |
| 64073 | 90085208 | 90087059 | 141013, 003249 |
| 64074 | 90088515 | 90089861 | 141013 |
| 64075 | 90112828 | 90114419 | 222019 |
| 64076 | 90124016 | 90124777 | 126856 |

The locations of ODSCs on Chromosome 17 are shown in Table 59.

TABLE 59
ODSC Locations on Chromosome 17

| SEQ ID NO. | Start | End | Overlapping Gene(s) |
|---|---|---|---|
| 64077 | 49580 | 50460 | |
| 64078 | 82884 | 83707 | 181031 |
| 64079 | 188555 | 189161 | 181031 |
| 64080 | 198044 | 199730 | 181031 |
| 64081 | 200373 | 204093 | 262294, 181031 |
| 64082 | 234802 | 236593 | 181031 |
| 64083 | 243321 | 244183 | |
| 64084 | 462502 | 465025 | 141252 |
| 64085 | 531544 | 532857 | 141252 |
| 64086 | 617238 | 619348 | 141252 |
| 64087 | 635293 | 636590 | 167695 |
| 64088 | 654637 | 656349 | 231784, 179409 |
| 64089 | 684461 | 686603 | 171861, 167699 |
| 64090 | 899650 | 901668 | 177370 |
| 64091 | 990708 | 991489 | 159842 |
| 64092 | 1012101 | 1012994 | 159842 |
| 64093 | 1162364 | 1163371 | |
| 64094 | 1170952 | 1172233 | |
| 64095 | 1293571 | 1294074 | 108953 |
| 64096 | 1301828 | 1303927 | 108953 |
| 64097 | 1315952 | 1317049 | |
| 64098 | 1357906 | 1361474 | 167193 |
| 64099 | 1363346 | 1364002 | 167193 |
| 64100 | 1371026 | 1371672 | 197879 |
| 64101 | 1372239 | 1372898 | 197879 |
| 64102 | 1387998 | 1390068 | 197879 |
| 64103 | 1391118 | 1392484 | 197879 |
| 64104 | 1393735 | 1395658 | 197879 |
| 64105 | 1418882 | 1420487 | 236618, 132376 |
| 64106 | 1448877 | 1449301 | 174238 |
| 64107 | 1449455 | 1450122 | 174238 |
| 64108 | 1464819 | 1466412 | 174238 |
| 64109 | 1480422 | 1481276 | 167703 |
| 64110 | 1493050 | 1494009 | 167703 |
| 64111 | 1531508 | 1533553 | 167703 |
| 64112 | 1551888 | 1554671 | 174231, 167705 |
| 64113 | 1572612 | 1573307 | 174231 |
| 64114 | 1587317 | 1588600 | 174231 |
| 64115 | 1612581 | 1614624 | 185561 |
| 64116 | 1616804 | 1622249 | 167716, 186594 |
| 64117 | 1627158 | 1629007 | 167716 |
| 64118 | 1638852 | 1641845 | 262791, 167716 |
| 64119 | 1647623 | 1648851 | 167711 |
| 64120 | 1664958 | 1668215 | 132386 |
| 64121 | 1673249 | 1673802 | 132386 |
| 64122 | 1676225 | 1677011 | 132386 |
| 64123 | 1686170 | 1687842 | 186532 |
| 64124 | 1689722 | 1691841 | 186532 |
| 64125 | 1713587 | 1714643 | 186532 |
| 64126 | 1732380 | 1733642 | 132383, 265504, 186532 |
| 64127 | 1755295 | 1756094 | 132383 |
| 64128 | 1813221 | 1813840 | |
| 64129 | 1849015 | 1849698 | 185924 |
| 64130 | 1898339 | 1902598 | 262445, 185924 |
| 64131 | 1927018 | 1929781 | 185924 |
| 64132 | 1933105 | 1934304 | 108963 |
| 64133 | 1944770 | 1946380 | 262664, 262533, 108963 |
| 64134 | 1952982 | 1954037 | 267195, 267200 |
| 64135 | 1998070 | 1998870 | 070366, 262810 |
| 64136 | 2060995 | 2061713 | 070366 |
| 64137 | 2075088 | 2076692 | 070366 |
| 64138 | 2077354 | 2078683 | 070366 |
| 64139 | 2080660 | 2081098 | 070366 |
| 64140 | 2115420 | 2118644 | 236838, 070366 |
| 64141 | 2125888 | 2127397 | 070366 |
| 64142 | 2205896 | 2207610 | 167720, 070366 |
| 64143 | 2212785 | 2213501 | 167720 |
| 64144 | 2238858 | 2241595 | 141258, 167721 |
| 64145 | 2251047 | 2251595 | 141258 |
| 64146 | 2286735 | 2287445 | 070444 |
| 64147 | 2295867 | 2297517 | 070444 |
| 64148 | 2301748 | 2305911 | 070444 |
| 64149 | 2388830 | 2389667 | 127804 |
| 64150 | 2413957 | 2415635 | 127804 |
| 64151 | 2448634 | 2449288 | |
| 64152 | 2496117 | 2498499 | 007168 |
| 64153 | 2553957 | 2554658 | 007168 |
| 64154 | 2612302 | 2616138 | 262050, 132361 |
| 64155 | 2907236 | 2908457 | 132359 |
| 64156 | 3377161 | 3378212 | 108381, 141255 |
| 64157 | 3378613 | 3380053 | 108381, 141255 |
| 64158 | 3415224 | 3417145 | 141255, 167723 |
| 64159 | 3465921 | 3466942 | |
| 64160 | 3537843 | 3538479 | 262304, 197417 |
| 64161 | 3538584 | 3540719 | 040531, 262304, 197417 |
| 64162 | 3550281 | 3551536 | 040531 |
| 64163 | 3562940 | 3564015 | 040531 |
| 64164 | 3570346 | 3573087 | 127774, 213977, 257950 |
| 64165 | 3626216 | 3627709 | 177602, 083457 |
| 64166 | 3705065 | 3708526 | 262194 |
| 64167 | 3748636 | 3749842 | 074356 |
| 64168 | 3879736 | 3880504 | 263312 |
| 64169 | 4045030 | 4048226 | 167740, 074755 |
| 64170 | 4050996 | 4051416 | 167740 |
| 64171 | 4112874 | 4113348 | 185722 |
| 64172 | 4123993 | 4124904 | 185722 |
| 64173 | 4166135 | 4167590 | 185722 |
| 64174 | 4213572 | 4214114 | 132388 |
| 64175 | 4268286 | 4270560 | 132388 |
| 64176 | 4357490 | 4357965 | 182557 |
| 64177 | 4385686 | 4387563 | 262823, 182557 |
| 64178 | 4399708 | 4402542 | 183018, 229782 |
| 64179 | 4424323 | 4424834 | 183018 |
| 64180 | 4437218 | 4438173 | 183018 |
| 64181 | 4457400 | 4459572 | 132382 |
| 64182 | 4476817 | 4477489 | |
| 64183 | 4568129 | 4568860 | |
| 64184 | 4582712 | 4583403 | 141456, 235085 |
| 64185 | 4606674 | 4608082 | 244184, 141456 |
| 64186 | 4612880 | 4614726 | 141480 |
| 64187 | 4615023 | 4616384 | 141480 |
| 64188 | 4622670 | 4625910 | 141480 |
| 64189 | 4633555 | 4635457 | 161920, 261898 |
| 64190 | 4639193 | 4640190 | 161921 |
| 64191 | 4642023 | 4643435 | 141497, 161921 |
| 64192 | 4662898 | 4663811 | |
| 64193 | 4674589 | 4676121 | 142484 |
| 64194 | 4690520 | 4694076 | 182327 |
| 64195 | 4698635 | 4700292 | 142507 |
| 64196 | 4709742 | 4711178 | 129219, 262165 |
| 64197 | 4736270 | 4737690 | 141503 |
| 64198 | 4801639 | 4803595 | 205710, 108556 |
| 64199 | 4811589 | 4813041 | |
| 64200 | 4817006 | 4817808 | |
| 64201 | 4822809 | 4824827 | |
| 64202 | 4842082 | 4845100 | 108523, 108528 |
| 64203 | 4847569 | 4854768 | 108515, 108518, 108523 |
| 64204 | 4869493 | 4874339 | 262429, 108509, 091640 |
| 64205 | 4889739 | 4891886 | 262227, 262678, 196388, |
| | | | 203562, 108509 |
| 64206 | 4900130 | 4902920 | 129250, 196388 |
| 64207 | 4934742 | 4935368 | |
| 64208 | 5014358 | 5015992 | 234327, 167840 |
| 64209 | 5094363 | 5095731 | 261879, 180626 |
| 64210 | 5124054 | 5125468 | 161929, 261879 |
| 64211 | 5185047 | 5186315 | 029725 |
| 64212 | 5217110 | 5217590 | 029725 |
| 64213 | 5308103 | 5309380 | 108559 |
| 64214 | 5321841 | 5324346 | 129197, 108559 |
| 64215 | 5341258 | 5342980 | 108561 |
| 64216 | 5371252 | 5372922 | 262099, 005100 |
| 64217 | 5388440 | 5390864 | 167842, 072849 |
| 64218 | 5419032 | 5420592 | 091592 |
| 64219 | 5723478 | 5725050 | 179314 |
| 64220 | 5740228 | 5740720 | 179314 |
| 64221 | 6542701 | 6545273 | 129235, 198920 |
| 64222 | 6554435 | 6556045 | 256806, 212734, 108590 |
| 64223 | 6603200 | 6604583 | 141485 |
| 64224 | 6613378 | 6617758 | 141485 |
| 64225 | 6619893 | 6620453 | |
| 64226 | 6654795 | 6657507 | |
| 64227 | 6658465 | 6660478 | 132530 |
| 64228 | 6679028 | 6679857 | 177294 |
| 64229 | 6699083 | 6702236 | 177294, 167858 |
| 64230 | 6725813 | 6726985 | 167858 |
| 64231 | 6756780 | 6758881 | 262943 |
| 64232 | 6770994 | 6772874 | 262943 |
| 64233 | 6810495 | 6810960 | 262943, 215067 |
| 64234 | 6830254 | 6831439 | 262503, 262943, 215067 |
| 64235 | 6914980 | 6919180 | 267532, 219200, 269871, |
| | | | 161939, 258315, 215067, |
| | | | 267047 |
| 64236 | 6922581 | 6924267 | 267532, 267047 |
| 64237 | 6924950 | 6926163 | |
| 64238 | 6938114 | 6940631 | 174327 |
| 64239 | 6940839 | 6942847 | 174327 |
| 64240 | 6945677 | 6947609 | 174326 |
| 64241 | 6963662 | 6964218 | |
| 64242 | 7001224 | 7001853 | |
| 64243 | 7016850 | 7019919 | 161944 |
| 64244 | 7035815 | 7036548 | |
| 64245 | 7038709 | 7040230 | |
| 64246 | 7059353 | 7060347 | |
| 64247 | 7067998 | 7068842 | |
| 64248 | 7070796 | 7071863 | |
| 64249 | 7080134 | 7083492 | 141505 |
| 64250 | 7122538 | 7124903 | 132535, 072778 |
| 64251 | 7136992 | 7138849 | 040633, 004975 |
| 64252 | 7140830 | 7143501 | 170296, 040633 |
| 64253 | 7144361 | 7147160 | 175826, 170296, 262526 |
| 64254 | 7153662 | 7156704 | 170291, 175826, 262302 |
| 64255 | 7161825 | 7165839 | 181885, 170291, 262302 |
| 64256 | 7166257 | 7167075 | 181885 |
| 64257 | 7183731 | 7185657 | 181856 |
| 64258 | 7197866 | 7198419 | 006047 |
| 64259 | 7199521 | 7200169 | |
| 64260 | 7209604 | 7212596 | 132507 |
| 64261 | 7217732 | 7219734 | 215041, 132522, 261915 |
| 64262 | 7231875 | 7233261 | 215041 |
| 64263 | 7253198 | 7256146 | 213859, 263171, 072818 |
| 64264 | 7283715 | 7284739 | 174292 |
| 64265 | 7297446 | 7298950 | 262481, 187838 |
| 64266 | 7306336 | 7308401 | 169992, 262481, 187838, |
| | | | 205544 |
| 64267 | 7338126 | 7340938 | 181284, 262624, 262880 |
| 64268 | 7343132 | 7344588 | 263301, 161958, 262880, |
| | | | 272884 |
| 64269 | 7348100 | 7349427 | 170175, 161958, 272884 |
| 64270 | 7359041 | 7360067 | 170175 |
| 64271 | 7370001 | 7370843 | 174282 |
| 64272 | 7382003 | 7383716 | 174282 |
| 64273 | 7386489 | 7388598 | 181222, 174282 |
| 64274 | 7461176 | 7462373 | 161955, 248871, 239697 |
| 64275 | 7463713 | 7466229 | 161956, 248871, 161955, |
| | | | 239697 |
| 64276 | 7475110 | 7477870 | 161960, 264772, 161956, |
| | | | 265500 |
| 64277 | 7482049 | 7484041 | 129226, 161960, 264772 |
| 64278 | 7485898 | 7488258 | 129255, 233223 |
| 64279 | 7516938 | 7519005 | 268503, 129214, 129245 |
| 64280 | 7529790 | 7531626 | 141504, 129214 |
| 64281 | 7534675 | 7535573 | 129214 |
| 64282 | 7588622 | 7592473 | 141499, 141510, 262251 |
| 64283 | 7620430 | 7620946 | 183914 |
| 64284 | 7736615 | 7742978 | 183914 |
| 64285 | 7743425 | 7749215 | 132510 |
| 64286 | 7759442 | 7762270 | 183011, 182224 |
| 64287 | 7788134 | 7789333 | 183011, 170004 |
| 64288 | 7818544 | 7819652 | 179859 |
| 64289 | 7834462 | 7835920 | 170037, 170043 |
| 64290 | 7893245 | 7893944 | |
| 64291 | 7919552 | 7921071 | 132518 |
| 64292 | 7922870 | 7923453 | 132518 |
| 64293 | 7935192 | 7935616 | |
| 64294 | 7962246 | 7963066 | |
| 64295 | 8020674 | 8024637 | 179111, 179148 |
| 64296 | 8028766 | 8029465 | |
| 64297 | 8054356 | 8060998 | 179094, 263620 |
| 64298 | 8065055 | 8067974 | 220205, 263620 |
| 64299 | 8076182 | 8077631 | 200463, 179029 |
| 64300 | 8079551 | 8080426 | 266824, 179029 |
| 64301 | 8089188 | 8091547 | 266638 |
| 64302 | 8092413 | 8093884 | 196544 |
| 64303 | 8113852 | 8114923 | 178999 |
| 64304 | 8123678 | 8128035 | 178977 |
| 64305 | 8140344 | 8141027 | 178971 |
| 64306 | 8150663 | 8153646 | 178921, 178971 |
| 64307 | 8191588 | 8192615 | 108961, 125434 |
| 64308 | 8198206 | 8199569 | 125434 |
| 64309 | 8279037 | 8280286 | 184619, 265749, 263809 |
| 64310 | 8285392 | 8287687 | 161970, 263809 |
| 64311 | 8288043 | 8288762 | |
| 64312 | 8323230 | 8324388 | 166579 |
| 64313 | 8333793 | 8335218 | 166579 |
| 64314 | 8338361 | 8340507 | 166579 |
| 64315 | 8380857 | 8381536 | 133026, 166579 |
| 64316 | 8435212 | 8436003 | 133026 |
| 64317 | 8445799 | 8447417 | 133026 |
| 64318 | 8533917 | 8535347 | 133026 |
| 64319 | 8615645 | 8616449 | |
| 64320 | 8648036 | 8649737 | 161973 |
| 64321 | 8924281 | 8926123 | 065320 |
| 64322 | 8959940 | 8961165 | 065320 |
| 64323 | 8964092 | 8965256 | 065320 |
| 64324 | 8983672 | 8985363 | 065320 |
| 64325 | 9007653 | 9008491 | 065320 |
| 64326 | 9073455 | 9074951 | 262966, 065320 |
| 64327 | 9109711 | 9110914 | 065320 |
| 64328 | 9120422 | 9121797 | 065320 |
| 64329 | 9143018 | 9144778 | 065320 |
| 64330 | 9201098 | 9202097 | 170310, 263708 |
| 64331 | 9263612 | 9265041 | 170310 |
| 64332 | 9292306 | 9292887 | 170310 |
| 64333 | 9478260 | 9479452 | 170310 |
| 64334 | 9547594 | 9549605 | 154914, 265349 |
| 64335 | 9551005 | 9552222 | 154914 |
| 64336 | 9580059 | 9581521 | 154914 |
| 64337 | 9745425 | 9746201 | 065325 |
| 64338 | 9989432 | 9989980 | 007237 |
| 64339 | 10523941 | 10524593 | 214970, 272975 |
| 64340 | 10599851 | 10602451 | 187824, 170222, 133028 |
| 64341 | 10627665 | 10629128 | 187824 |
| 64342 | 10631907 | 10635009 | 263400, 187824 |
| 64343 | 10640127 | 10641589 | 263400 |
| 64344 | 10647608 | 10648209 | 263400, 264016 |
| 64345 | 10659100 | 10661087 | 263400, 264016 |
| 64346 | 10702744 | 10703259 | 263400, 263429 |
| 64347 | 10747873 | 10749087 | |
| 64348 | 11511624 | 11512563 | 007174 |
| 64349 | 11608098 | 11608988 | 007174 |
| 64350 | 11899763 | 11901645 | 154957 |
| 64351 | 11923909 | 11925355 | 065559, 241185 |
| 64352 | 11926831 | 11928650 | 065559 |
| 64353 | 12886975 | 12888730 | 006740 |
| 64354 | 12893218 | 12894274 | 263707, 006740 |
| 64355 | 12920125 | 12922368 | 006744 |
| 64356 | 13307354 | 13308155 | |
| 64357 | 13309041 | 13309789 | |
| 64358 | 13313991 | 13315028 | |
| 64359 | 13318838 | 13319889 | |
| 64360 | 13323256 | 13324678 | |
| 64361 | 13628276 | 13629409 | |
| 64362 | 13660108 | 13660534 | 236088 |
| 64363 | 13679389 | 13679960 | 236088 |
| 64364 | 13717084 | 13717662 | 236088 |
| 64365 | 13909271 | 13910267 | 236088 |
| 64366 | 13914615 | 13915416 | 236088 |
| 64367 | 13956034 | 13957373 | 236088 |
| 64368 | 13972092 | 13973700 | 006695, 236088 |
| 64369 | 14108554 | 14110370 | 006695 |
| 64370 | 14111849 | 14113798 | 006695 |
| 64371 | 14200333 | 14201334 | 262786 |
| 64372 | 14202256 | 14208339 | 266709, 125430 |
| 64373 | 14210146 | 14213000 | 125430 |
| 64374 | 14224224 | 14225337 | 125430 |
| 64375 | 14228814 | 14229585 | 125430 |
| 64376 | 14233566 | 14235059 | 125430 |
| 64377 | 14275517 | 14279697 | 230647 |
| 64378 | 14373651 | 14374817 | |
| 64379 | 14639638 | 14643339 | |
| 64380 | 14659711 | 14660547 | |
| 64381 | 14663434 | 14664757 | |
| 64382 | 14778976 | 14779717 | 238212 |
| 64383 | 15336370 | 15337147 | |
| 64384 | 15337480 | 15338246 | |
| 64385 | 15339599 | 15340665 | 239704, 259024 |
| 64386 | 15553163 | 15553797 | 221926, 251537 |
| 64387 | 15901738 | 15904099 | 011295, 214941 |
| 64388 | 15930372 | 15932603 | 141027, 011295 |
| 64389 | 15940756 | 15941964 | 141027, 011295 |
| 64390 | 15943281 | 15944788 | 227782, 141027, 011295 |
| 64391 | 16117854 | 16119912 | 141027 |
| 64392 | 16120097 | 16121360 | 108474, 141027 |
| 64393 | 16158474 | 16159445 | 108474 |
| 64394 | 16188416 | 16191748 | 108474 |
| 64395 | 16199857 | 16202299 | 108474 |
| 64396 | 16216427 | 16217274 | 108474 |
| 64397 | 16255474 | 16257270 | 166582 |
| 64398 | 16281129 | 16282874 | |
| 64399 | 16283706 | 16285252 | 170315 |
| 64400 | 16310233 | 16310836 | |
| 64401 | 16341230 | 16344058 | 266651, 175061 |
| 64402 | 16438607 | 16440689 | |
| 64403 | 16496966 | 16497431 | |
| 64404 | 16556335 | 16557393 | 264765, 197566 |
| 64405 | 16944070 | 16945831 | |
| 64406 | 16945874 | 16947453 | 133030 |
| 64407 | 16954799 | 16956042 | 133030 |
| 64408 | 16980797 | 16983785 | 225442, 133030 |
| 64409 | 16990126 | 16991885 | 133030 |
| 64410 | 17054349 | 17056270 | 133030 |
| 64411 | 17058598 | 17060695 | 133030 |
| 64412 | 17108571 | 17110346 | 179598, 264187, 133030 |
| 64413 | 17138775 | 17141593 | 266498, 154803, 264187 |
| 64414 | 17183858 | 17185005 | 141030 |
| 64415 | 17206393 | 17207313 | 205309 |
| 64416 | 17290760 | 17291467 | |
| 64417 | 17308749 | 17312860 | |
| 64418 | 17318837 | 17323247 | |
| 64419 | 17343073 | 17344971 | |
| 64420 | 17346443 | 17351296 | |
| 64421 | 17380043 | 17383632 | 141026 |
| 64422 | 17395854 | 17400718 | 108551, 141026 |
| 64423 | 17432428 | 17433315 | 133027 |
| 64424 | 17438806 | 17442997 | 133027 |
| 64425 | 17462589 | 17466105 | 133027 |
| 64426 | 17471682 | 17472668 | 133027 |
| 64427 | 17472961 | 17476192 | 133027 |
| 64428 | 17483600 | 17486294 | 133027 |
| 64429 | 17493714 | 17496113 | 264666, 133027 |
| 64430 | 17584250 | 17587055 | 108557 |
| 64431 | 17596474 | 17598971 | 108557 |
| 64432 | 17614064 | 17614809 | 108557 |
| 64433 | 17713174 | 17714497 | 072310, 108557 |
| 64434 | 17722910 | 17724383 | 072310 |
| 64435 | 17726506 | 17729444 | 072310 |
| 64436 | 17733252 | 17734931 | 072310 |
| 64437 | 17738210 | 17742059 | 072310 |
| 64438 | 17742540 | 17744864 | |
| 64439 | 17763176 | 17764021 | 175662 |
| 64440 | 17780347 | 17781754 | 175662 |
| 64441 | 17803734 | 17804891 | 175662 |
| 64442 | 17824156 | 17826648 | 175662 |
| 64443 | 17838848 | 17839565 | 175662 |
| 64444 | 17875065 | 17876353 | 171962, 175662 |
| 64445 | 17886414 | 17886972 | 171953, 171962 |
| 64446 | 17941645 | 17944788 | 141034, 171953 |
| 64447 | 17990555 | 17992311 | 108591 |
| 64448 | 18018798 | 18019977 | 091536 |
| 64449 | 18020392 | 18021716 | 091536 |
| 64450 | 18063037 | 18063687 | 091536 |
| 64451 | 18084625 | 18085038 | 266677 |
| 64452 | 18085326 | 18089507 | 091542, 266677 |
| 64453 | 18106202 | 18107512 | 091542 |
| 64454 | 18128838 | 18129746 | 131899 |
| 64455 | 18160487 | 18162690 | 177731 |
| 64456 | 18163667 | 18165613 | 177427 |
| 64457 | 18217564 | 18219551 | 176994, 177302 |
| 64458 | 18259819 | 18261491 | 176974 |
| 64459 | 18265037 | 18267123 | 176974 |
| 64460 | 18761059 | 18762416 | 141127 |
| 64461 | 18770260 | 18771182 | 141127 |
| 64462 | 18822952 | 18825810 | 141127 |
| 64463 | 18883322 | 18886793 | 188522, 154025 |
| 64464 | 18890406 | 18894153 | 188522, 154025 |
| 64465 | 18903102 | 18905084 | 188522, 154025 |
| 64466 | 18905375 | 18909713 | 188522, 154025 |
| 64467 | 19140563 | 19141952 | 072134 |
| 64468 | 19241002 | 19242270 | 108641 |
| 64469 | 19248581 | 19251448 | 108641 |
| 64470 | 19264906 | 19266734 | 108641 |
| 64471 | 19280237 | 19282650 | 166484, 108641 |
| 64472 | 19410670 | 19411932 | 142494 |
| 64473 | 19417073 | 19418236 | 142494 |
| 64474 | 19429141 | 19430321 | 142494 |
| 64475 | 19436722 | 19438407 | 142494 |
| 64476 | 19441522 | 19442802 | 142494 |
| 64477 | 19453030 | 19453627 | 142494 |
| 64478 | 19548600 | 19553292 | 072210, 264932 |
| 64479 | 19553763 | 19555796 | 072210 |
| 64480 | 19555858 | 19556479 | 072210 |
| 64481 | 19559429 | 19560232 | 072210 |
| 64482 | 19560463 | 19561014 | 072210 |
| 64483 | 19563483 | 19564370 | 072210 |
| 64484 | 19574781 | 19576314 | 072210 |
| 64485 | 19607176 | 19607898 | 180638 |
| 64486 | 19621468 | 19622245 | 180638 |
| 64487 | 19622417 | 19622993 | 262681 |
| 64488 | 19623996 | 19624630 | 262681 |
| 64489 | 19630338 | 19632081 | |
| 64490 | 19651247 | 19652203 | 108602 |
| 64491 | 19655421 | 19657610 | |
| 64492 | 19669603 | 19670683 | |
| 64493 | 19769842 | 19771580 | 083290 |
| 64494 | 19806352 | 19809348 | 108599 |
| 64495 | 19843941 | 19844618 | 108599 |
| 64496 | 19858982 | 19859579 | 108599 |
| 64497 | 19861264 | 19861805 | 108599 |
| 64498 | 19880042 | 19882033 | 108599 |
| 64499 | 20059291 | 20060407 | 128487 |
| 64500 | 20811489 | 20812770 | 233098 |
| 64501 | 20932297 | 20933559 | 124422 |
| 64502 | 20944686 | 20947451 | 124422 |
| 64503 | 21029691 | 21031379 | 109016 |
| 64504 | 21064928 | 21066082 | 109016 |
| 64505 | 21116588 | 21118915 | 235530, 178307 |
| 64506 | 21155728 | 21157177 | 154035 |
| 64507 | 21178779 | 21179701 | |
| 64508 | 21187494 | 21193707 | 034152 |
| 64509 | 21219687 | 21220619 | |
| 64510 | 21414327 | 21415516 | |
| 64511 | 25571280 | 25572830 | |
| 64512 | 25574096 | 25575113 | |
| 64513 | 25592270 | 25592821 | |
| 64514 | 25620303 | 25622890 | 109046, 263583 |
| 64515 | 25658779 | 25661275 | 266313 |
| 64516 | 25666870 | 25667665 | 266313 |
| 64517 | 25675606 | 25676677 | |
| 64518 | 25679533 | 25681929 | 265683 |
| 64519 | 25689411 | 25690423 | |
| 64520 | 25782778 | 25784010 | 141068 |
| 64521 | 25787059 | 25787841 | 141068 |
| 64522 | 25821093 | 25822673 | 141068 |
| 64523 | 25867338 | 25868091 | 141068 |
| 64524 | 25878577 | 25880505 | 141068 |
| 64525 | 25885471 | 25886690 | 141068 |
| 64526 | 25935193 | 25935727 | 141068 |
| 64527 | 26127444 | 26128115 | 007171, 266202 |
| 64528 | 26132352 | 26133369 | 266202 |
| 64529 | 26172302 | 26173273 | 266202 |
| 64530 | 26187848 | 26190049 | 266202 |
| 64531 | 26219671 | 26220800 | 266202, 232859 |
| 64532 | 26368368 | 26369736 | 087095 |
| 64533 | 26384803 | 26385386 | 087095 |
| 64534 | 26460459 | 26461128 | 087095 |
| 64535 | 26578346 | 26579092 | |
| 64536 | 26585259 | 26586131 | 264486 |
| 64537 | 26609526 | 26610738 | 265480 |
| 64538 | 26651202 | 26651728 | 109084 |
| 64539 | 26661833 | 26664282 | 109079, 109083 |
| 64540 | 26672194 | 26675418 | 004142, 109079 |
| 64541 | 26683346 | 26685527 | 244045, 258924, 004142 |
| 64542 | 26694870 | 26698995 | 273171, 244045, 109072, |
| | | | 004139, 258924 |
| 64543 | 26725020 | 26726177 | 076351, 004139 |
| 64544 | 26731515 | 26733625 | 265254, 076351 |
| 64545 | 26880449 | 26881251 | 258472, 087111 |
| 64546 | 26897742 | 26898995 | 087111, 258472 |
| 64547 | 26971002 | 26973079 | 007202 |
| 64548 | 26988214 | 26990321 | 109111, 132581 |
| 64549 | 27024477 | 27026501 | 109111 |
| 64550 | 27037480 | 27038643 | 167525 |
| 64551 | 27046307 | 27048326 | 198242, 238423 |
| 64552 | 27051904 | 27056518 | 267729, 160602, 160606 |
| 64553 | 27069869 | 27074344 | 265474, 076604, 265073, |
| | | | 160602 |
| 64554 | 27134885 | 27135369 | 173065 |
| 64555 | 27139108 | 27139950 | 173065 |
| 64556 | 27168857 | 27170065 | 173065 |
| 64557 | 27181419 | 27182728 | 132591, 173065 |
| 64558 | 27223378 | 27225678 | 167536, 266642, 132589 |
| 64559 | 27229213 | 27230488 | 167536 |
| 64560 | 27235761 | 27236255 | 109118 |
| 64561 | 27264339 | 27264775 | 109118, 265845 |
| 64562 | 27275393 | 27279831 | 179761, 109118 |
| 64563 | 27318656 | 27319443 | 063015, 179761 |
| 64564 | 27319874 | 27321424 | 063015, 179761 |
| 64565 | 27369427 | 27371156 | 179761 |
| 64566 | 27372100 | 27373888 | 179761 |
| 64567 | 27434937 | 27436371 | 196535 |
| 64568 | 27459070 | 27461162 | 196535 |
| 64569 | 27475169 | 27477460 | 196535, 263709 |
| 64570 | 27480837 | 27484010 | 263709, 196535 |
| 64571 | 27484230 | 27484877 | 196535 |
| 64572 | 27485576 | 27486181 | 196535 |
| 64573 | 27493447 | 27493915 | 196535 |
| 64574 | 27502997 | 27508193 | 196535 |
| 64575 | 27564809 | 27566223 | |
| 64576 | 27567055 | 27568647 | |
| 64577 | 27618314 | 27622200 | 108256 |
| 64578 | 27716846 | 27718824 | 160551, 263719, 264808, |
| | | | 266111 |
| 64579 | 27777008 | 27777960 | 160551 |
| 64580 | 27882710 | 27883357 | |
| 64581 | 27884058 | 27884924 | |
| 64582 | 27892667 | 27896477 | 167543, 168792, 264031 |
| 64583 | 27899286 | 27900284 | 167543, 264031 |
| 64584 | 27901596 | 27902501 | 108262, 264031 |
| 64585 | 27915678 | 27917347 | 198720, 108262, 264031 |
| 64586 | 27940261 | 27941254 | 264031, 198720 |
| 64587 | 27966105 | 27966900 | 263370, 141298, 264031 |
| 64588 | 28071569 | 28072238 | 141298 |
| 64589 | 28087897 | 28088726 | 263657, 141298 |
| 64590 | 28246132 | 28246822 | 141298 |
| 64591 | 28255851 | 28258846 | 176927, 141298 |
| 64592 | 28439987 | 28440861 | 265394 |
| 64593 | 28443081 | 28444856 | 266919, 199071, 265394, |
| | | | 126653 |
| 64594 | 28583664 | 28584258 | 108578 |
| 64595 | 28589991 | 28592593 | 108578 |
| 64596 | 28663240 | 28664152 | |
| 64597 | 28671204 | 28672436 | 207132 |
| 64598 | 28705204 | 28707302 | 108582 |
| 64599 | 28777217 | 28778226 | 108582 |
| 64600 | 28803842 | 28805286 | 108587 |
| 64601 | 28926917 | 28928361 | 248121, 214719 |
| 64602 | 29150977 | 29152516 | 176390 |
| 64603 | 29158528 | 29159390 | 176208 |
| 64604 | 29232454 | 29234132 | 184060, 172171 |
| 64605 | 29298017 | 29298623 | 181481 |
| 64606 | 29379180 | 29380181 | 265798, 266865 |
| 64607 | 29394545 | 29395572 | 266865 |
| 64608 | 29421447 | 29423241 | 196712, 264107 |
| 64609 | 29596635 | 29597249 | 196712 |
| 64610 | 29600951 | 29602500 | 126861, 196712 |
| 64611 | 29752059 | 29753128 | 131242 |
| 64612 | 29802205 | 29803270 | 131242 |
| 64613 | 29814626 | 29816027 | 131242 |
| 64614 | 29816567 | 29817366 | 131242 |
| 64615 | 29826830 | 29827526 | 131242 |
| 64616 | 29875734 | 29877758 | |
| 64617 | 29885566 | 29890173 | 207614 |
| 64618 | 29891443 | 29893076 | |
| 64619 | 29894613 | 29896149 | |
| 64620 | 29897947 | 29899423 | 228768 |
| 64621 | 29907127 | 29909248 | |
| 64622 | 29922799 | 29925019 | 266877 |
| 64623 | 29925194 | 29926842 | 266877 |
| 64624 | 29958529 | 29959634 | 266877 |
| 64625 | 30081026 | 30082101 | |
| 64626 | 30130280 | 30132955 | |
| 64627 | 30134845 | 30135299 | |
| 64628 | 30168847 | 30170588 | |
| 64629 | 30185311 | 30186516 | 172301 |
| 64630 | 30227915 | 30229290 | 108651 |
| 64631 | 30334159 | 30335197 | 185158 |
| 64632 | 30450815 | 30452908 | |
| 64633 | 30468226 | 30470624 | 126858, 214708 |
| 64634 | 30667864 | 30669667 | 108666, 265794 |
| 64635 | 30676069 | 30678949 | 010244, 207928, 108666, |
| | | | 265794 |
| 64636 | 30771126 | 30773248 | 108671 |
| 64637 | 30813114 | 30814004 | 176749 |
| 64638 | 31121360 | 31122320 | 176658 |
| 64639 | 31149389 | 31150058 | 176658 |
| 64640 | 31203217 | 31204789 | 226377, 176658 |
| 64641 | 31254504 | 31255781 | 006042 |
| 64642 | 31841281 | 31841847 | 108684 |
| 64643 | 32561769 | 32563952 | |
| 64644 | 32575155 | 32577232 | |
| 64645 | 32578829 | 32580590 | |
| 64646 | 32581645 | 32583331 | 108691, 268024 |
| 64647 | 33287546 | 33289576 | 198783, 132141 |
| 64648 | 33306562 | 33308910 | 005156, 132141 |
| 64649 | 33348823 | 33350183 | 267618, 092871 |
| 64650 | 33389321 | 33392427 | 267618, 092871 |
| 64651 | 33401395 | 33403089 | 267618, 092871 |
| 64652 | 33408145 | 33410587 | 267618, 092871 |
| 64653 | 33412499 | 33417337 | 092871, 267618 |
| 64654 | 33445977 | 33447234 | 267618, 185379 |
| 64655 | 33468164 | 33469588 | 073536 |
| 64656 | 33477942 | 33478535 | 141161 |
| 64657 | 33568609 | 33573429 | 166750, 266947 |
| 64658 | 33581205 | 33581885 | 166750 |
| 64659 | 33759717 | 33760981 | 172123 |
| 64660 | 33894574 | 33895885 | 267321 |
| 64661 | 33904704 | 33906578 | 006125, 108733 |
| 64662 | 33913480 | 33915391 | 006125 |
| 64663 | 34039596 | 34040747 | 006125 |
| 64664 | 34043808 | 34045071 | 006125 |
| 64665 | 34110348 | 34111156 | 129270 |
| 64666 | 34135099 | 34137477 | 172660, 267578 |
| 64667 | 34216938 | 34217886 | |
| 64668 | 34239649 | 34241305 | 266972 |
| 64669 | 34242140 | 34242600 | 266972 |
| 64670 | 34299007 | 34301885 | |
| 64671 | 34302366 | 34303354 | |
| 64672 | 34304527 | 34309604 | 161573 |
| 64673 | 34327094 | 34329624 | 267566, 161574, 267596, |
| | | | 213494 |
| 64674 | 34333827 | 34334536 | 267566 |
| 64675 | 34842095 | 34843302 | 108278 |
| 64676 | 34889947 | 34892162 | 184886, 141140 |
| 64677 | 34899653 | 34902676 | 005955 |
| 64678 | 34940114 | 34940795 | 005955 |
| 64679 | 34947720 | 34949450 | 108272 |
| 64680 | 34957182 | 34959317 | 129282, 108272 |
| 64681 | 35305456 | 35307076 | 108270 |
| 64682 | 35381400 | 35382200 | 108270 |
| 64683 | 35388704 | 35389494 | 108270 |
| 64684 | 35424715 | 35425588 | |
| 64685 | 35464147 | 35464919 | 132142 |
| 64686 | 35504184 | 35504920 | 132142 |
| 64687 | 35596537 | 35597596 | 132142 |
| 64688 | 35715143 | 35717417 | 132142 |
| 64689 | 35759164 | 35759940 | 132142 |
| 64690 | 35766023 | 35768089 | 108264, 267613, 132142 |
| 64691 | 35836378 | 35837491 | 108264 |
| 64692 | 35847890 | 35851162 | 161326 |
| 64693 | 35861989 | 35862693 | 161326 |
| 64694 | 35968587 | 35970323 | 141141, 267542, 006114 |
| 64695 | 35989228 | 35989744 | 141141 |
| 64696 | 36003968 | 36004370 | 267668 |
| 64697 | 36031086 | 36033310 | 267668 |
| 64698 | 36035233 | 36036616 | 267668 |
| 64699 | 36043345 | 36044013 | 267668 |
| 64700 | 36057538 | 36060049 | 108753 |
| 64701 | 36068027 | 36069385 | 108753 |
| 64702 | 36072461 | 36074252 | 108753 |
| 64703 | 36076278 | 36079226 | 108753 |
| 64704 | 36101493 | 36106240 | 259549, 108753 |
| 64705 | 36183945 | 36184926 | |
| 64706 | 36506967 | 36508836 | 174111 |
| 64707 | 36713286 | 36714859 | 017373 |
| 64708 | 36740461 | 36742072 | 017373 |
| 64709 | 36761733 | 36763947 | 017373 |
| 64710 | 36769978 | 36770822 | |
| 64711 | 36830344 | 36831756 | 179294 |
| 64712 | 36832692 | 36833784 | |
| 64713 | 36849944 | 36850816 | |
| 64714 | 36856387 | 36862677 | 108292, 265930, 261005, |
| | | | 266040 |
| 64715 | 36886068 | 36887903 | 230055 |
| 64716 | 36903658 | 36906506 | 227450, 056661 |
| 64717 | 36907229 | 36909841 | 108294 |
| 64718 | 36954800 | 36956612 | 141720 |
| 64719 | 36980695 | 36982720 | 263791, 108296 |
| 64720 | 37008848 | 37010753 | 199293, 125691 |
| 64721 | 37023585 | 37024399 | |
| 64722 | 37024945 | 37029510 | 002834 |
| 64723 | 37029821 | 37030418 | 002834 |
| 64724 | 37035885 | 37037145 | 002834 |
| 64725 | 37041485 | 37042231 | 002834 |
| 64726 | 37053337 | 37055355 | 002834 |
| 64727 | 37056820 | 37057650 | 002834 |
| 64728 | 37308758 | 37311045 | 161381 |
| 64729 | 37353377 | 37354286 | 067191 |
| 64730 | 37355656 | 37357845 | 108298 |
| 64731 | 37394664 | 37395289 | 265460 |
| 64732 | 37404538 | 37405224 | 265460 |
| 64733 | 37412360 | 37413586 | |
| 64734 | 37481664 | 37482219 | 108306 |
| 64735 | 37556425 | 37558861 | 266469, 108306 |
| 64736 | 37606691 | 37608450 | 125686 |
| 64737 | 37609820 | 37610580 | |
| 64738 | 37617210 | 37619909 | 167258 |
| 64739 | 37773428 | 37774818 | |
| 64740 | 37792549 | 37794418 | 131748, 131771 |
| 64741 | 37843179 | 37845295 | 141736, 161395 |
| 64742 | 37851407 | 37852373 | 161395, 141736 |
| 64743 | 37855878 | 37857478 | 141736 |
| 64744 | 37858946 | 37860387 | 141736 |
| 64745 | 37861385 | 37862763 | 141736 |
| 64746 | 37885741 | 37887215 | 141741, 141736 |
| 64747 | 37893455 | 37897671 | 141738 |
| 64748 | 37905942 | 37907261 | |
| 64749 | 37909730 | 37911748 | |
| 64750 | 37984401 | 37985216 | 161405 |
| 64751 | 38020042 | 38020838 | 161405 |
| 64752 | 38060735 | 38061614 | 073605 |
| 64753 | 38077689 | 38087342 | 264968, 172057 |
| 64754 | 38089809 | 38091206 | 264968 |
| 64755 | 38095068 | 38097321 | 264968 |
| 64756 | 38099658 | 38101263 | 204913 |
| 64757 | 38106813 | 38107565 | |
| 64758 | 38108446 | 38110274 | |
| 64759 | 38136104 | 38138680 | 108344 |
| 64760 | 38188488 | 38189338 | 008838 |
| 64761 | 38194997 | 38195538 | 008838 |
| 64762 | 38218811 | 38220023 | 126351 |
| 64763 | 38231615 | 38232421 | 126351 |
| 64764 | 38253700 | 38258628 | 126368 |
| 64765 | 38263690 | 38265500 | |
| 64766 | 38266152 | 38271847 | |
| 64767 | 38277173 | 38280228 | 188895 |
| 64768 | 38295384 | 38297726 | 108349 |
| 64769 | 38333481 | 38334556 | 108352 |
| 64770 | 38375057 | 38376302 | 171475 |
| 64771 | 38407501 | 38408212 | 171475 |
| 64772 | 38443611 | 38444273 | 094804 |
| 64773 | 38465441 | 38466145 | 131759 |
| 64774 | 38467863 | 38472231 | 131759 |
| 64775 | 38472441 | 38475175 | 131759 |
| 64776 | 38482295 | 38483532 | 131759 |
| 64777 | 38486263 | 38489120 | 131759 |
| 64778 | 38497224 | 38503496 | 265666, 131759 |
| 64779 | 38507299 | 38510221 | 131759 |
| 64780 | 38518729 | 38520186 | 183153, 266208 |
| 64781 | 38574043 | 38574716 | 131747 |
| 64782 | 38598941 | 38602522 | 141753 |
| 64783 | 38602759 | 38605976 | 141753 |
| 64784 | 38606784 | 38610926 | 141753 |
| 64785 | 38632055 | 38632849 | 131746 |
| 64786 | 38666235 | 38666848 | |
| 64787 | 38674312 | 38675904 | 266088 |
| 64788 | 38689208 | 38690230 | |
| 64789 | 38693916 | 38695886 | |
| 64790 | 38698193 | 38700325 | |
| 64791 | 38703593 | 38709334 | |
| 64792 | 38754131 | 38755581 | |
| 64793 | 38803283 | 38805534 | 073584, 264058 |
| 64794 | 38974493 | 38976301 | 167920, 186395 |
| 64795 | 39010373 | 39010882 | 265359 |
| 64796 | 39056966 | 39060290 | |
| 64797 | 39070497 | 39073072 | |
| 64798 | 39108605 | 39112064 | 234477 |
| 64799 | 39112114 | 39114810 | 196859, 234477 |
| 64800 | 39572020 | 39573011 | 234859 |
| 64801 | 39801635 | 39802550 | 173801 |
| 64802 | 39821521 | 39823825 | 173801 |
| 64803 | 39834522 | 39835745 | 173801 |
| 64804 | 39843629 | 39846845 | 173812, 173801 |
| 64805 | 39853505 | 39853989 | 173801 |
| 64806 | 39928112 | 39930710 | 173801 |
| 64807 | 39933449 | 39934902 | 173801 |
| 64808 | 39941461 | 39943229 | 173801 |
| 64809 | 39967291 | 39968457 | 141696 |
| 64810 | 39991726 | 39992792 | 161594, 141698 |
| 64811 | 40020742 | 40022072 | 178502 |
| 64812 | 40025560 | 40026470 | 131473 |
| 64813 | 40050888 | 40052424 | 131473 |
| 64814 | 40064274 | 40064846 | 131473 |
| 64815 | 40070331 | 40071185 | 131473 |
| 64816 | 40074088 | 40076755 | 131473 |
| 64817 | 40086032 | 40086938 | 204815, 131473 |
| 64818 | 40094041 | 40094910 | 204815 |
| 64819 | 40117619 | 40119857 | 173786, 204815 |
| 64820 | 40124594 | 40125510 | 173786 |
| 64821 | 40141546 | 40142228 | 168259 |
| 64822 | 40154486 | 40155748 | 168259 |
| 64823 | 40168429 | 40170605 | 168259, 168256 |
| 64824 | 40171867 | 40172848 | 168259, 168256 |
| 64825 | 40175199 | 40177435 | 168256 |
| 64826 | 40201809 | 40203254 | 187595 |
| 64827 | 40274488 | 40275560 | 197723, 267261 |
| 64828 | 40287060 | 40288885 | 108774, 267261 |
| 64829 | 40305489 | 40307607 | 267658, 108774, 267261 |
| 64830 | 40335642 | 40336580 | 161610 |
| 64831 | 40400439 | 40401146 | 173757 |
| 64832 | 40421377 | 40421888 | 267758, 173757 |
| 64833 | 40427400 | 40429495 | 173757 |
| 64834 | 40439756 | 40441183 | 126561 |
| 64835 | 40463524 | 40464625 | 126561 |
| 64836 | 40476472 | 40478263 | 168610 |
| 64837 | 40480598 | 40481989 | 168610 |
| 64838 | 40530897 | 40531410 | 168610 |
| 64839 | 40538841 | 40541606 | 168610 |
| 64840 | 40549945 | 40551107 | 221020 |
| 64841 | 40573674 | 40575439 | 177469 |
| 64842 | 40580296 | 40580944 | |
| 64843 | 40610227 | 40612659 | 033627 |
| 64844 | 40615297 | 40616406 | 033627 |
| 64845 | 40636373 | 40636820 | 033627 |
| 64846 | 40670036 | 40673231 | 033627 |
| 64847 | 40679192 | 40680287 | |
| 64848 | 40681746 | 40682477 | |
| 64849 | 40682512 | 40683311 | |
| 64850 | 40683662 | 40684469 | |
| 64851 | 40687353 | 40689759 | 266929, 108784 |
| 64852 | 40692681 | 40694245 | 266929, 108784 |
| 64853 | 40696607 | 40697433 | 266929 |
| 64854 | 40705752 | 40707635 | 266962, 108786, 266929 |
| 64855 | 40713341 | 40716086 | 068120, 266929 |
| 64856 | 40718685 | 40720364 | 108788 |
| 64857 | 40729381 | 40730925 | 131470 |
| 64858 | 40760119 | 40762194 | 131462, 141699 |
| 64859 | 40810850 | 40812151 | 037042 |
| 64860 | 40818779 | 40820547 | 068137, 037042 |
| 64861 | 40827109 | 40832240 | 267042, 184451, 068137 |
| 64862 | 40895821 | 40897332 | 108799 |
| 64863 | 40924868 | 40926878 | 131475 |
| 64864 | 40949394 | 40951005 | 176563, 183978 |
| 64865 | 40961282 | 40961797 | 176563 |
| 64866 | 40975364 | 40976688 | 131467, 126581 |
| 64867 | 40984404 | 40986649 | 126581, 131467 |
| 64868 | 41001240 | 41002266 | 131480 |
| 64869 | 41007242 | 41007654 | 131471 |
| 64870 | 41016082 | 41017021 | |
| 64871 | 41017474 | 41019467 | 260105 |
| 64872 | 41030184 | 41030686 | 213373 |
| 64873 | 41038020 | 41039465 | 213373 |
| 64874 | 41045782 | 41047776 | 213373 |
| 64875 | 41052206 | 41054470 | 131482 |
| 64876 | 41055638 | 41056528 | 131482 |
| 64877 | 41070659 | 41071528 | |
| 64878 | 41080324 | 41081745 | |
| 64879 | 41115771 | 41117237 | 266967, 108825 |
| 64880 | 41132259 | 41134035 | 198863, 108825, 267060 |
| 64881 | 41149765 | 41151947 | 131469, 200127 |
| 64882 | 41158518 | 41159414 | 068079 |
| 64883 | 41173034 | 41174591 | 108828 |
| 64884 | 41176288 | 41177369 | 108830, 108828 |
| 64885 | 41192627 | 41193121 | |
| 64886 | 41220557 | 41221742 | 012048 |
| 64887 | 41236333 | 41237264 | 012048 |
| 64888 | 41277296 | 41278675 | 198496, 012048 |
| 64889 | 41321665 | 41324662 | 188554, 267340, 267002 |
| 64890 | 41392211 | 41393790 | |
| 64891 | 41437620 | 41439346 | |
| 64892 | 41444905 | 41447694 | 188825 |
| 64893 | 41465830 | 41466503 | 188825 |
| 64894 | 41476020 | 41477722 | 175906 |
| 64895 | 41478113 | 41480347 | 175906 |
| 64896 | 41486903 | 41487618 | |
| 64897 | 41506479 | 41507283 | |
| 64898 | 41521485 | 41522752 | 267151 |
| 64899 | 41526727 | 41527651 | 267151 |
| 64900 | 41533457 | 41535825 | |
| 64901 | 41543219 | 41544277 | |
| 64902 | 41553000 | 41553631 | 237888 |
| 64903 | 41557080 | 41557805 | |
| 64904 | 41560086 | 41562308 | 067596 |
| 64905 | 41621680 | 41622537 | 267747, 067596, 175832 |
| 64906 | 41622673 | 41623679 | 267747, 175832 |
| 64907 | 41797379 | 41799483 | 267253 |
| 64908 | 41801306 | 41801781 | |
| 64909 | 41849219 | 41851105 | 108861 |
| 64910 | 41854934 | 41857100 | 108861 |
| 64911 | 41892058 | 41892458 | 161647 |
| 64912 | 41909495 | 41910858 | 161647 |
| 64913 | 41916776 | 41917847 | |
| 64914 | 42071928 | 42072667 | 131096 |
| 64915 | 42078134 | 42080805 | 131096 |
| 64916 | 42081229 | 42084626 | 161653, 131096 |
| 64917 | 42091216 | 42092741 | 091947 |
| 64918 | 42143114 | 42144835 | 161654 |
| 64919 | 42147219 | 42149096 | 141349 |
| 64920 | 42174552 | 42175594 | 108840 |
| 64921 | 42185420 | 42187062 | 108840 |
| 64922 | 42187624 | 42188970 | 108840 |
| 64923 | 42200091 | 42201968 | 108840 |
| 64924 | 42214916 | 42215943 | |
| 64925 | 42218877 | 42219962 | 125319 |
| 64926 | 42263262 | 42265271 | 168591, 267080 |
| 64927 | 42275589 | 42278644 | 267394, 087152 |
| 64928 | 42295203 | 42300387 | 260793, 108312 |
| 64929 | 42300624 | 42301699 | 260793 |
| 64930 | 42401162 | 42403245 | 013306 |
| 64931 | 42404223 | 42404652 | |
| 64932 | 42408485 | 42409358 | 267211 |
| 64933 | 42422166 | 42423448 | 030582 |
| 64934 | 42579791 | 42581842 | 186566 |
| 64935 | 42664630 | 42665296 | |
| 64936 | 42766196 | 42768102 | 180329, 180336 |
| 64937 | 42785072 | 42786220 | 161692 |
| 64938 | 42900082 | 42900621 | 182963 |
| 64939 | 42976077 | 42977486 | 167131, 214447, 108883 |
| 64940 | 42993235 | 42994788 | 131095 |
| 64941 | 43017392 | 43018538 | 186185 |
| 64942 | 43056843 | 43060060 | 267788 |
| 64943 | 43093270 | 43094623 | |
| 64944 | 43095957 | 43097080 | |
| 64945 | 43098580 | 43099998 | |
| 64946 | 43127793 | 43129155 | 136448, 172992 |
| 64947 | 43137409 | 43139601 | 172992, 136448 |
| 64948 | 43169362 | 43169804 | 136448 |
| 64949 | 43170085 | 43171721 | 136448 |
| 64950 | 43206729 | 43207760 | 161714 |
| 64951 | 43209072 | 43210895 | 181513, 161714 |
| 64952 | 43212581 | 43214051 | 181513 |
| 64953 | 43214828 | 43215870 | 181513 |
| 64954 | 43221176 | 43222774 | 181513 |
| 64955 | 43223656 | 43228586 | 186834, 224505 |
| 64956 | 43237369 | 43240238 | 168517, 224505 |
| 64957 | 43247505 | 43250812 | 267288 |
| 64958 | 43274721 | 43275779 | 267121 |
| 64959 | 43278965 | 43279764 | 267121 |
| 64960 | 43324227 | 43325895 | 267278, 184922, 233483 |
| 64961 | 43343142 | 43343578 | 006062, 267278 |
| 64962 | 43350526 | 43352150 | 006062 |
| 64963 | 43354499 | 43355482 | 006062 |
| 64964 | 43361345 | 43362119 | 006062 |
| 64965 | 43367208 | 43367820 | 006062 |
| 64966 | 43369784 | 43372409 | 006062 |
| 64967 | 43383274 | 43386148 | 006062 |
| 64968 | 43387964 | 43391863 | 006062 |
| 64969 | 43393058 | 43394906 | 006062 |
| 64970 | 43448077 | 43450343 | 267446 |
| 64971 | 43502436 | 43503541 | 159314 |
| 64972 | 43782696 | 43783242 | 120088, 263715 |
| 64973 | 43971045 | 43972070 | 186868, 264589 |
| 64974 | 44004438 | 44005083 | 186868 |
| 64975 | 44019390 | 44020501 | 186868 |
| 64976 | 44267357 | 44272735 | 214401, 120071 |
| 64977 | 44279645 | 44280464 | 120071 |
| 64978 | 44343918 | 44345365 | 261575 |
| 64979 | 44835897 | 44836781 | |
| 64980 | 44847799 | 44850784 | 108379 |
| 64981 | 44913070 | 44914304 | 158955 |
| 64982 | 44928533 | 44929124 | 158955 |
| 64983 | 44999848 | 45001512 | 262633, 108433, 261886 |
| 64984 | 45061551 | 45062652 | 262879, 262633, 108433, |
| | | | 263142 |
| 64985 | 45144030 | 45145240 | 262879 |
| 64986 | 45176937 | 45177898 | 262879 |
| 64987 | 45265720 | 45267712 | 004897 |
| 64988 | 45301971 | 45303612 | |
| 64989 | 45306464 | 45309374 | |
| 64990 | 45329368 | 45330420 | |
| 64991 | 45330596 | 45332376 | 259753, 259207 |
| 64992 | 45342348 | 45345399 | 259753, 259207 |
| 64993 | 45382430 | 45383653 | 263293, 259753, 259207 |
| 64994 | 45391723 | 45395185 | 263293, 259753, 259207 |
| 64995 | 45400050 | 45402039 | 178852, 263293, 259753, |
| | | | 259207 |
| 64996 | 45697611 | 45698455 | 141279, 263766 |
| 64997 | 45726085 | 45728885 | 108424, 263766 |
| 64998 | 45770947 | 45772394 | 198933 |
| 64999 | 45908167 | 45909104 | 141294, 159111 |
| 65000 | 45917701 | 45919770 | 141295 |
| 65001 | 45939713 | 45940904 | 264243 |
| 65002 | 45972264 | 45974735 | 234494, 167182, 264920 |
| 65003 | 46018091 | 46020252 | 108439, 234494 |
| 65004 | 46022529 | 46024792 | 263798, 264019, 108439 |
| 65005 | 46025484 | 46027731 | 264019, 108439, 263798 |
| 65006 | 46029386 | 46029962 | 263798, 167183 |
| 65007 | 46101092 | 46104354 | 005243 |
| 65008 | 46114098 | 46115481 | 207947, 005243 |
| 65009 | 46124291 | 46127753 | 082641, 263412 |
| 65010 | 46127799 | 46129725 | 082641 |
| 65011 | 46130004 | 46131678 | 082641 |
| 65012 | 46145671 | 46146530 | |
| 65013 | 46177959 | 46179162 | 108468 |
| 65014 | 46184221 | 46185936 | 002919 |
| 65015 | 46204601 | 46205422 | |
| 65016 | 46230921 | 46232092 | 141293 |
| 65017 | 46238719 | 46241862 | 141293 |
| 65018 | 46342179 | 46343692 | 141293 |
| 65019 | 46662465 | 46663108 | 120093, 233101 |
| 65020 | 46868246 | 46869992 | 170703 |
| 65021 | 46871925 | 46874082 | 170703 |
| 65022 | 46876545 | 46877282 | 170703 |
| 65023 | 46882156 | 46882529 | 170703 |
| 65024 | 46907989 | 46909067 | 136436 |
| 65025 | 46948586 | 46949929 | |
| 65026 | 46968789 | 46971191 | 159199, 248278 |
| 65027 | 46975358 | 46975659 | 248278 |
| 65028 | 46985009 | 46987590 | 159202, 248278 |
| 65029 | 46992177 | 46993621 | 159202 |
| 65030 | 47021340 | 47022924 | 159210 |
| 65031 | 47049602 | 47050229 | |
| 65032 | 47183982 | 47184643 | |
| 65033 | 47268942 | 47271183 | |
| 65034 | 47436912 | 47440013 | 248714, 198740 |
| 65035 | 47491472 | 47492758 | 167085, 262039 |
| 65036 | 47591873 | 47593458 | 064300, 249906 |
| 65037 | 47628742 | 47629187 | 249906, 250310 |
| 65038 | 47645001 | 47646204 | 249906, 250310 |
| 65039 | 47646923 | 47647947 | 249906, 250310 |
| 65040 | 47688402 | 47688974 | 121067 |
| 65041 | 47723179 | 47724845 | 121067 |
| 65042 | 47754109 | 47755897 | 121067 |
| 65043 | 47784284 | 47786086 | 250751, 121073 |
| 65044 | 47807346 | 47808665 | 121104 |
| 65045 | 47822512 | 47823315 | 121104 |
| 65046 | 47840510 | 47841548 | 121104 |
| 65047 | 47841552 | 47841963 | 121104 |
| 65048 | 47865076 | 47866740 | 136504, 121104 |
| 65049 | 48105577 | 48106580 | |
| 65050 | 48132863 | 48133400 | 005884, 246640 |
| 65051 | 48172347 | 48173450 | 005882 |
| 65052 | 48178553 | 48180205 | 250282, 005882 |
| 65053 | 48222065 | 48222771 | 108819, 236472 |
| 65054 | 48224087 | 48229927 | 108819 |
| 65055 | 48254866 | 48256327 | 188662 |
| 65056 | 48276750 | 48278820 | 108821 |
| 65057 | 48295976 | 48296561 | |
| 65058 | 48349567 | 48351468 | 167105 |
| 65059 | 48358925 | 48359663 | 251179 |
| 65060 | 48366642 | 48368601 | |
| 65061 | 48422418 | 48424289 | 015532 |
| 65062 | 48448477 | 48450907 | 154920, 108826 |
| 65063 | 48473521 | 48475260 | 253102, 108829 |
| 65064 | 48502485 | 48504121 | 167107 |
| 65065 | 48506051 | 48507050 | 167107 |
| 65066 | 48517574 | 48518188 | 167107 |
| 65067 | 48536146 | 48536908 | 167107 |
| 65068 | 48543656 | 48544865 | 167107, 136457 |
| 65069 | 48553767 | 48554247 | |
| 65070 | 48555224 | 48557128 | 136444 |
| 65071 | 48559730 | 48560782 | 136444 |
| 65072 | 48592485 | 48593118 | 136449 |
| 65073 | 48624114 | 48625376 | 006282 |
| 65074 | 48705123 | 48705916 | 251239 |
| 65075 | 48711302 | 48720341 | 108846 |
| 65076 | 48737262 | 48741910 | 108846 |
| 65077 | 48744601 | 48746728 | 108846 |
| 65078 | 48747982 | 48748915 | 108846 |
| 65079 | 48750065 | 48751086 | 108846 |
| 65080 | 48752749 | 48753700 | 108846 |
| 65081 | 48761723 | 48765527 | 108846 |
| 65082 | 48768635 | 48773515 | 262967, 154945, 108846 |
| 65083 | 48783982 | 48786737 | 154945 |
| 65084 | 48796167 | 48798028 | 108848 |
| 65085 | 48842703 | 48844300 | 167117 |
| 65086 | 48863141 | 48863861 | |
| 65087 | 48882866 | 48884063 | |
| 65088 | 48929123 | 48931298 | |
| 65089 | 48940369 | 48941255 | 141232 |
| 65090 | 48941608 | 48945987 | 229980, 141232 |
| 65091 | 48961301 | 48962306 | 229980 |
| 65092 | 48966679 | 48967739 | 229980 |
| 65093 | 48968037 | 48969412 | 229980 |
| 65094 | 48981464 | 48983984 | 229980 |
| 65095 | 48986425 | 48988411 | 262006, 229980 |
| 65096 | 48992257 | 48993893 | |
| 65097 | 48996922 | 48997496 | |
| 65098 | 49008111 | 49011263 | |
| 65099 | 49012436 | 49013794 | 247011 |
| 65100 | 49017069 | 49018032 | 247011 |
| 65101 | 49020614 | 49022821 | 251665, 247011 |
| 65102 | 49026365 | 49028687 | |
| 65103 | 49171132 | 49171607 | 008294 |
| 65104 | 49175315 | 49176151 | 238815, 008294 |
| 65105 | 49180332 | 49180918 | 008294 |
| 65106 | 49189879 | 49190467 | 008294 |
| 65107 | 49195474 | 49196122 | 008294 |
| 65108 | 49196799 | 49200402 | 008294 |
| 65109 | 49229770 | 49232240 | 239672, 011052, 243678 |
| 65110 | 49242821 | 49245301 | 011052, 243678 |
| 65111 | 49309241 | 49310069 | 011258 |
| 65112 | 49336417 | 49338668 | 011260, 011258 |
| 65113 | 49500713 | 49501632 | |
| 65114 | 49512858 | 49513688 | 267452 |
| 65115 | 49636755 | 49637849 | |
| 65116 | 49722180 | 49723103 | 154975 |
| 65117 | 49780355 | 49781574 | 154975 |
| 65118 | 49784607 | 49785420 | 154975 |
| 65119 | 49787160 | 49788340 | 154975 |
| 65120 | 50310643 | 50311987 | |
| 65121 | 50321754 | 50322554 | |
| 65122 | 50496394 | 50497258 | 263317 |
| 65123 | 50901794 | 50902278 | |
| 65124 | 51539619 | 51540103 | |
| 65125 | 52001781 | 52002521 | |
| 65126 | 52958249 | 52960197 | |
| 65127 | 52977603 | 52979833 | 141198 |
| 65128 | 52980591 | 52981294 | 141198 |
| 65129 | 53045004 | 53047247 | 166263, 166260 |
| 65130 | 53140382 | 53140746 | 166263 |
| 65131 | 53234351 | 53234908 | 166263 |
| 65132 | 53315167 | 53316753 | |
| 65133 | 53341734 | 53343778 | 108924 |
| 65134 | 53344330 | 53346862 | 108924 |
| 65135 | 53350020 | 53352533 | 263096, 108924 |
| 65136 | 53353662 | 53354471 | 108924 |
| 65137 | 53360175 | 53365671 | 108924 |
| 65138 | 53379390 | 53380071 | 108924 |
| 65139 | 53484726 | 53486089 | 108960 |
| 65140 | 53487961 | 53489382 | 108960 |
| 65141 | 53497486 | 53499600 | 108960 |
| 65142 | 53509760 | 53512411 | |
| 65143 | 53522032 | 53523251 | |
| 65144 | 53523512 | 53524669 | |
| 65145 | 53529036 | 53530919 | |
| 65146 | 53532118 | 53533318 | 262395 |
| 65147 | 53535560 | 53537573 | 262395 |
| 65148 | 53572505 | 53574356 | 261873 |
| 65149 | 53608147 | 53609278 | 236319 |
| 65150 | 53617858 | 53618599 | 236319 |
| 65151 | 53660959 | 53662141 | |
| 65152 | 53727237 | 53728008 | |
| 65153 | 53737662 | 53738429 | |
| 65154 | 53770506 | 53771361 | |
| 65155 | 53803660 | 53804479 | 166292 |
| 65156 | 53807712 | 53810617 | 166292 |
| 65157 | 53823789 | 53824394 | |
| 65158 | 53824519 | 53825018 | |
| 65159 | 53828176 | 53829526 | 141179 |
| 65160 | 53839317 | 53840377 | 141179 |
| 65161 | 53891406 | 53892271 | 141179 |
| 65162 | 54185949 | 54186549 | |
| 65163 | 54219703 | 54220353 | 153930 |
| 65164 | 54225584 | 54226524 | 153930 |
| 65165 | 54226911 | 54229144 | 153930 |
| 65166 | 54896907 | 54897735 | 214226 |
| 65167 | 54900038 | 54900625 | 214226 |
| 65168 | 54910175 | 54912159 | 153933, 214226 |
| 65169 | 54945311 | 54945834 | 153933 |
| 65170 | 54987050 | 54988257 | 121060 |
| 65171 | 54989019 | 54994139 | 121060 |
| 65172 | 55015052 | 55015618 | 121058 |
| 65173 | 55037678 | 55038724 | 121058 |
| 65174 | 55055182 | 55055758 | 121064 |
| 65175 | 55112503 | 55114096 | |
| 65176 | 55131364 | 55132090 | |
| 65177 | 55150913 | 55151581 | |
| 65178 | 55161685 | 55164249 | 121057, 263004 |
| 65179 | 55185692 | 55187379 | 121057 |
| 65180 | 55332919 | 55334964 | 153944 |
| 65181 | 55356032 | 55356594 | 153944 |
| 65182 | 55447466 | 55448093 | 153944 |
| 65183 | 55449494 | 55450935 | 153944 |
| 65184 | 55454500 | 55455016 | 153944 |
| 65185 | 55535685 | 55537180 | 153944 |
| 65186 | 55593484 | 55597255 | 153944 |
| 65187 | 55599691 | 55603592 | 266100, 153944 |
| 65188 | 55608632 | 55610008 | 153944 |
| 65189 | 55910338 | 55911280 | 265542 |
| 65190 | 55926436 | 55928360 | 181610 |
| 65191 | 55969702 | 55971357 | 180891 |
| 65192 | 55979910 | 55981030 | 180891 |
| 65193 | 56024268 | 56024884 | 180891 |
| 65194 | 56025956 | 56027037 | 180891 |
| 65195 | 56031747 | 56033206 | 180891 |
| 65196 | 56052332 | 56052990 | 136451 |
| 65197 | 56064161 | 56066628 | 264112, 266086, 136451 |
| 65198 | 56083222 | 56085428 | 136450 |
| 65199 | 56159927 | 56162422 | 264364, 266290 |
| 65200 | 56295817 | 56297528 | 167419, 011143 |
| 65201 | 56428740 | 56430885 | 108375, 213246, 265148 |
| 65202 | 56441827 | 56442850 | 265148, 108375 |
| 65203 | 56458246 | 56459005 | 265148, 108375 |
| 65204 | 56461273 | 56462137 | 265148, 108375 |
| 65205 | 56468629 | 56469309 | 265148, 108375 |
| 65206 | 56490277 | 56492429 | 265148, 108375 |
| 65207 | 56494057 | 56495535 | 108375 |
| 65208 | 56583731 | 56586083 | 108389 |
| 65209 | 56591258 | 56592938 | 108389 |
| 65210 | 56594179 | 56597371 | 264672, 108389 |
| 65211 | 56612072 | 56613490 | 108387, 264672 |
| 65212 | 56654000 | 56655330 | 121101 |
| 65213 | 56707941 | 56710224 | 212195, 121101 |
| 65214 | 56716884 | 56718138 | 121101 |
| 65215 | 56736045 | 56737197 | 202077, 121101 |
| 65216 | 56769174 | 56771135 | 108384, 121101 |
| 65217 | 56818392 | 56818988 | |
| 65218 | 56832864 | 56833655 | 175175 |
| 65219 | 56883209 | 56884107 | 175175 |
| 65220 | 56990624 | 56992062 | 175175 |
| 65221 | 57068498 | 57071592 | 108395 |
| 65222 | 57078835 | 57079372 | 108395 |
| 65223 | 57081509 | 57082780 | 108395 |
| 65224 | 57086074 | 57086464 | 108395 |
| 65225 | 57183519 | 57184770 | 224738, 108395 |
| 65226 | 57231888 | 57233032 | 068489, 182628 |
| 65227 | 57287044 | 57288264 | 265303, 167447 |
| 65228 | 57297726 | 57298950 | 153982 |
| 65229 | 57374047 | 57375108 | |
| 65230 | 57403745 | 57411077 | 175155 |
| 65231 | 57434180 | 57436226 | 175155 |
| 65232 | 57442271 | 57446467 | 263857, 175155 |
| 65233 | 57448814 | 57449776 | 175155 |
| 65234 | 57498029 | 57503607 | |
| 65235 | 57503718 | 57513481 | 265313 |
| 65236 | 57516655 | 57519569 | 265313 |
| 65237 | 57542244 | 57543568 | 265313 |
| 65238 | 57591574 | 57592286 | 265313 |
| 65239 | 57598616 | 57599411 | 265313 |
| 65240 | 57642522 | 57643730 | 108406 |
| 65241 | 57694516 | 57698725 | 141367 |
| 65242 | 57783831 | 57786560 | 062716, 141378 |
| 65243 | 57803027 | 57803493 | 062716 |
| 65244 | 57806672 | 57807091 | 062716 |
| 65245 | 57827120 | 57835454 | 062716 |
| 65246 | 57859885 | 57865585 | 267637, 062716 |
| 65247 | 57904938 | 57909520 | 062716 |
| 65248 | 57910249 | 57910847 | 062716 |
| 65249 | 57913102 | 57919310 | 199004, 062716 |
| 65250 | 57919360 | 57927419 | 201524, 062716 |
| 65251 | 57928579 | 57932570 | |
| 65252 | 57932684 | 57934608 | |
| 65253 | 57969304 | 57971504 | 108443, 108423 |
| 65254 | 58041025 | 58042949 | 267302, 189050, 267318, |
| | | | 267104 |
| 65255 | 58114320 | 58114978 | |
| 65256 | 58155430 | 58156594 | 068097 |
| 65257 | 58219966 | 58220993 | |
| 65258 | 58379616 | 58380484 | 170832 |
| 65259 | 58402779 | 58403907 | 170832 |
| 65260 | 58433092 | 58433880 | 170832 |
| 65261 | 58450029 | 58450829 | 170832 |
| 65262 | 58467541 | 58470822 | 141371, 170832 |
| 65263 | 58498479 | 58499585 | 170832, 141371 |
| 65264 | 58602370 | 58604543 | 259349, 062725 |
| 65265 | 58637220 | 58637825 | |
| 65266 | 58676678 | 58679800 | 170836 |
| 65267 | 58682149 | 58682807 | 170836 |
| 65268 | 58719178 | 58719633 | 170836 |
| 65269 | 58754520 | 58755984 | 141376 |
| 65270 | 58807294 | 58808186 | 141376 |
| 65271 | 59142344 | 59143495 | 267207, 141376 |
| 65272 | 59153590 | 59154674 | 141376 |
| 65273 | 59278483 | 59279124 | 141376 |
| 65274 | 59413213 | 59414207 | 141376 |
| 65275 | 59487994 | 59490741 | 187013, 267280 |
| 65276 | 59566492 | 59567300 | |
| 65277 | 59940198 | 59941805 | 136492 |
| 65278 | 60004271 | 60005722 | 108506 |
| 65279 | 60107577 | 60107984 | 108510 |
| 65280 | 60141597 | 60143927 | 108510 |
| 65281 | 60167415 | 60168121 | |
| 65282 | 60169914 | 60172079 | |
| 65283 | 60178517 | 60179469 | |
| 65284 | 60266004 | 60266546 | |
| 65285 | 60555634 | 60556309 | 146872 |
| 65286 | 60729511 | 60730823 | 011028 |
| 65287 | 60780463 | 60781982 | 265702, 173838 |
| 65288 | 60896824 | 60897717 | 265000 |
| 65289 | 61043001 | 61044514 | 266411, 263931 |
| 65290 | 61522732 | 61524665 | 008283 |
| 65291 | 61626741 | 61628929 | 136485 |
| 65292 | 61677388 | 61679885 | 136463 |
| 65293 | 61698779 | 61701092 | 198909 |
| 65294 | 61758723 | 61759214 | 198909 |
| 65295 | 61818023 | 61820311 | 266173, 125695 |
| 65296 | 61849838 | 61852820 | 198231, 108588 |
| 65297 | 61903559 | 61906026 | 087191, 108592 |
| 65298 | 61915879 | 61916294 | 108604 |
| 65299 | 61918759 | 61921229 | 108604 |
| 65300 | 61926302 | 61926911 | 240280 |
| 65301 | 62133686 | 62134454 | 178607 |
| 65302 | 62134775 | 62136345 | 178607 |
| 65303 | 62151195 | 62153623 | 178607 |
| 65304 | 62158431 | 62163553 | 178607 |
| 65305 | 62191997 | 62194462 | 178607 |
| 65306 | 62205273 | 62207834 | 178607 |
| 65307 | 62221947 | 62226578 | 199753, 266402, 136478 |
| 65308 | 62227311 | 62228296 | 136478 |
| 65309 | 62233238 | 62234003 | 136478 |
| 65310 | 62255168 | 62256055 | 136478 |
| 65311 | 62263821 | 62265326 | 136478 |
| 65312 | 62337258 | 62337628 | 136478 |
| 65313 | 62339214 | 62341397 | 136478 |
| 65314 | 62492424 | 62494626 | 256525 |
| 65315 | 62501039 | 62503934 | 258890, 108654 |
| 65316 | 62530933 | 62531510 | 258890 |
| 65317 | 62657610 | 62658841 | 108854 |
| 65318 | 62700237 | 62701396 | |
| 65319 | 62701980 | 62704173 | |
| 65320 | 62737135 | 62737626 | |
| 65321 | 62970463 | 62972894 | 214174 |
| 65322 | 62988376 | 62989304 | |
| 65323 | 63009953 | 63010352 | 120063 |
| 65324 | 63049893 | 63055229 | 120063 |
| 65325 | 63057517 | 63061876 | |
| 65326 | 63070754 | 63071470 | |
| 65327 | 63083724 | 63084287 | |
| 65328 | 63087853 | 63089674 | |
| 65329 | 63095006 | 63097746 | 263470 |
| 65330 | 63118437 | 63120454 | |
| 65331 | 63126313 | 63131019 | |
| 65332 | 63132519 | 63133730 | 108370 |
| 65333 | 63150966 | 63151699 | 108370 |
| 65334 | 63186864 | 63191636 | 108370 |
| 65335 | 63542683 | 63544020 | 266076, 168646 |
| 65336 | 63555854 | 63558351 | 168646, 266076 |
| 65337 | 63746690 | 63747260 | 154240 |
| 65338 | 63779896 | 63780589 | 154240 |
| 65339 | 63781101 | 63781811 | 154240 |
| 65340 | 64223897 | 64224891 | 091583 |
| 65341 | 64225116 | 64226561 | 091583 |
| 65342 | 64228931 | 64229740 | 091583 |
| 65343 | 64231421 | 64232124 | 091583 |
| 65344 | 64271728 | 64273146 | |
| 65345 | 64297711 | 64301301 | 154229 |
| 65346 | 64320708 | 64321166 | 154229 |
| 65347 | 64337559 | 64338128 | 154229 |
| 65348 | 64339174 | 64340929 | 154229 |
| 65349 | 64344198 | 64346039 | 154229 |
| 65350 | 64411280 | 64412375 | 154229, 264630 |
| 65351 | 64413264 | 64413838 | 154229 |
| 65352 | 64417956 | 64418572 | 154229 |
| 65353 | 64447363 | 64448530 | 154229 |
| 65354 | 64455271 | 64457315 | 154229 |
| 65355 | 64461845 | 64465655 | 154229 |
| 65356 | 64499073 | 64499747 | 154229 |
| 65357 | 64502767 | 64506004 | 154229 |
| 65358 | 64513170 | 64514713 | 154229 |
| 65359 | 64657230 | 64658146 | 154229 |
| 65360 | 64900014 | 64901998 | |
| 65361 | 64947530 | 64949075 | |
| 65362 | 65091132 | 65092070 | 198265 |
| 65363 | 65146710 | 65148411 | 198265 |
| 65364 | 65174600 | 65175165 | 198265 |
| 65365 | 65240353 | 65243497 | 266473, 198265 |
| 65366 | 65256236 | 65257102 | |
| 65367 | 65276022 | 65276974 | |
| 65368 | 65285457 | 65286111 | |
| 65369 | 65360369 | 65363087 | 197170 |
| 65370 | 65373146 | 65375852 | 154217 |
| 65371 | 65386358 | 65387587 | 154217 |
| 65372 | 65397892 | 65399187 | 154217 |
| 65373 | 65416789 | 65419959 | 154217 |
| 65374 | 65483619 | 65484835 | 154217 |
| 65375 | 65524875 | 65525604 | 154217 |
| 65376 | 65713076 | 65715030 | 130935 |
| 65377 | 65775085 | 65775871 | |
| 65378 | 65820853 | 65822522 | 171634 |
| 65379 | 65988481 | 65991727 | 266176, 186665 |
| 65380 | 66015478 | 66017201 | |
| 65381 | 66031228 | 66032665 | 182481 |
| 65382 | 66097426 | 66098578 | 237854, 265055 |
| 65383 | 66201342 | 66202462 | |
| 65384 | 66243244 | 66245174 | 196704, 265100 |
| 65385 | 66287174 | 66288594 | 108932, 141337 |
| 65386 | 66373272 | 66375563 | 141337 |
| 65387 | 66378856 | 66381626 | 141337 |
| 65388 | 66387229 | 66388204 | 141337 |
| 65389 | 66452783 | 66454614 | 070540, 267009 |
| 65390 | 66470270 | 66470956 | 267009 |
| 65391 | 66507268 | 66509700 | 108946, 267009 |
| 65392 | 66527229 | 66528313 | 108946 |
| 65393 | 66575319 | 66577456 | 108950, 267461 |
| 65394 | 66580853 | 66581579 | 108950 |
| 65395 | 66581638 | 66582297 | 108950 |
| 65396 | 66591272 | 66593016 | 267659, 108950 |
| 65397 | 66593819 | 66598284 | 108950, 267659 |
| 65398 | 66609790 | 66610547 | 267659 |
| 65399 | 66657752 | 66658747 | 267659 |
| 65400 | 66755077 | 66756529 | 267659 |
| 65401 | 66788859 | 66791626 | 267250 |
| 65402 | 66815563 | 66816264 | |
| 65403 | 66816593 | 66818514 | |
| 65404 | 66820846 | 66821238 | |
| 65405 | 66839525 | 66840103 | |
| 65406 | 66934020 | 66934806 | 141338 |
| 65407 | 67043360 | 67043974 | 154258 |
| 65408 | 67117642 | 67119255 | 154262 |
| 65409 | 67126816 | 67128175 | 154262 |
| 65410 | 67136425 | 67139187 | 154262 |
| 65411 | 67264106 | 67265941 | 154265 |
| 65412 | 67321595 | 67324354 | 154265 |
| 65413 | 67386986 | 67387826 | |
| 65414 | 67602904 | 67604706 | 227517 |
| 65415 | 67757415 | 67759053 | 227517 |
| 65416 | 67759644 | 67761477 | 227517 |
| 65417 | 68683382 | 68684093 | |
| 65418 | 68859237 | 68860893 | |
| 65419 | 68867254 | 68868317 | |
| 65420 | 68934283 | 68935214 | |
| 65421 | 69038813 | 69039941 | |
| 65422 | 69081248 | 69081897 | |
| 65423 | 69159294 | 69160261 | 260785 |
| 65424 | 70026378 | 70027082 | 256124, 228639 |
| 65425 | 70072063 | 70072644 | 226101, 234899 |
| 65426 | 70075590 | 70076373 | 226101, 234899 |
| 65427 | 70088582 | 70089432 | 226101, 234899 |
| 65428 | 70115248 | 70116985 | 234899 |
| 65429 | 70122613 | 70123892 | 234899 |
| 65430 | 70132766 | 70133901 | 234899 |
| 65431 | 70147410 | 70148548 | 234899 |
| 65432 | 70179220 | 70179968 | 234899 |
| 65433 | 70334996 | 70335846 | 227036 |
| 65434 | 70417217 | 70419844 | 227036 |
| 65435 | 70441026 | 70441973 | 227036 |
| 65436 | 70679499 | 70681016 | 133195 |
| 65437 | 71025971 | 71027708 | 133195 |
| 65438 | 71088180 | 71089181 | 133195 |
| 65439 | 71187535 | 71189940 | 166685, 264860 |
| 65440 | 71197100 | 71198054 | 166685 |
| 65441 | 71227090 | 71229730 | 141219, 133193 |
| 65442 | 71284558 | 71285942 | 179604 |
| 65443 | 71286758 | 71290660 | 179604 |
| 65444 | 71294348 | 71295122 | 179604 |
| 65445 | 71300821 | 71302059 | 179604 |
| 65446 | 71303776 | 71305015 | 179604 |
| 65447 | 71305353 | 71309041 | 179604 |
| 65448 | 71653533 | 71654278 | |
| 65449 | 71714737 | 71715598 | |
| 65450 | 71743425 | 71744344 | 263574 |
| 65451 | 71765514 | 71767189 | 177338 |
| 65452 | 71791159 | 71795953 | 234721, 177338 |
| 65453 | 71796255 | 71796942 | 234721, 177338 |
| 65454 | 72188882 | 72189921 | |
| 65455 | 72199446 | 72201077 | 172809 |
| 65456 | 72206319 | 72206920 | 172809, 246731 |
| 65457 | 72208283 | 72209952 | 141540, 246731 |
| 65458 | 72369633 | 72370580 | |
| 65459 | 72375434 | 72376159 | |
| 65460 | 72422652 | 72424332 | 170412 |
| 65461 | 72425506 | 72430409 | 170412 |
| 65462 | 72438581 | 72441371 | 170412 |
| 65463 | 72442710 | 72444268 | 170412 |
| 65464 | 72449121 | 72451422 | |
| 65465 | 72732352 | 72734113 | 172794 |
| 65466 | 72744402 | 72748903 | 264624, 109062, 266036 |
| 65467 | 72753637 | 72757490 | 109062 |
| 65468 | 72759851 | 72760546 | 109062 |
| 65469 | 72762960 | 72767762 | 109065, 109062 |
| 65470 | 72769181 | 72770631 | 109065 |
| 65471 | 72771629 | 72773461 | 109066, 109065 |
| 65472 | 72868494 | 72870521 | 161513 |
| 65473 | 72889386 | 72890249 | 172782 |
| 65474 | 72977850 | 72978792 | |
| 65475 | 73007836 | 73009573 | 167862 |
| 65476 | 73029678 | 73032048 | 180901 |
| 65477 | 73042272 | 73044248 | 167863, 180901 |
| 65478 | 73072819 | 73076154 | 232724 |
| 65479 | 73079825 | 73080829 | |
| 65480 | 73081667 | 73082149 | |
| 65481 | 73092204 | 73092764 | 170190 |
| 65482 | 73105869 | 73106801 | 125449 |
| 65483 | 73119765 | 73121696 | 125449 |
| 65484 | 73126520 | 73128221 | 125458 |
| 65485 | 73148908 | 73150895 | 189159 |
| 65486 | 73177509 | 73179738 | 188612 |
| 65487 | 73200398 | 73202528 | 125450 |
| 65488 | 73256713 | 73258882 | 125445, 125447 |
| 65489 | 73265884 | 73268031 | 263843, 125457 |
| 65490 | 73284817 | 73286607 | 125454 |
| 65491 | 73304669 | 73306016 | |
| 65492 | 73389521 | 73390801 | 265342, 177885 |
| 65493 | 73400171 | 73402599 | 264511, 177885 |
| 65494 | 73422344 | 73422988 | |
| 65495 | 73451524 | 73453714 | 177728 |
| 65496 | 73510756 | 73513060 | 182173, 177303 |
| 65497 | 73521530 | 73522600 | 073350 |
| 65498 | 73552944 | 73554114 | 073350 |
| 65499 | 73557898 | 73558505 | 073350 |
| 65500 | 73584575 | 73586171 | 266714 |
| 65501 | 73606080 | 73607650 | 266714 |
| 65502 | 73641607 | 73643198 | 259120, 108469 |
| 65503 | 73662373 | 73664247 | 161526, 108469 |
| 65504 | 73749409 | 73751142 | 132470, 108479 |
| 65505 | 73760082 | 73761789 | 108479 |
| 65506 | 73765520 | 73766291 | |
| 65507 | 73772164 | 73773039 | 132475 |
| 65508 | 73774599 | 73776327 | 132475 |
| 65509 | 73779093 | 73782825 | 132478, 263565, 132475 |
| 65510 | 73840491 | 73841704 | 092929 |
| 65511 | 73846576 | 73848308 | 132471 |
| 65512 | 73848976 | 73852988 | 132471 |
| 65513 | 73868371 | 73869682 | |
| 65514 | 73871496 | 73875236 | 267801, 132481 |
| 65515 | 73891387 | 73893356 | 267342, 141569 |
| 65516 | 73900106 | 73902215 | 267426, 204316 |
| 65517 | 73936383 | 73937572 | 188878 |
| 65518 | 73971794 | 73973162 | 161533 |
| 65519 | 73974380 | 73976535 | 261408, 257949, 161533 |
| 65520 | 73977772 | 73978793 | 261408, 257949 |
| 65521 | 73989133 | 73989988 | 261408, 257949 |
| 65522 | 73992709 | 73995163 | 261408, 257949 |
| 65523 | 73995899 | 73996481 | 261408, 257949 |
| 65524 | 73999222 | 73999838 | 250506, 261408 |
| 65525 | 74067819 | 74069455 | 186919, 167881 |
| 65526 | 74098943 | 74100736 | 182473 |
| 65527 | 74143606 | 74144218 | 267128, 141576 |
| 65528 | 74178098 | 74178700 | 141576 |
| 65529 | 74192776 | 74193127 | 141576 |
| 65530 | 74200217 | 74200774 | 141576 |
| 65531 | 74236677 | 74237280 | |
| 65532 | 74247242 | 74249115 | |
| 65533 | 74254470 | 74256602 | |
| 65534 | 74258763 | 74259486 | |
| 65535 | 74260114 | 74263133 | 185262 |
| 65536 | 74264217 | 74264654 | 185262 |
| 65537 | 74347641 | 74350613 | 161542 |
| 65538 | 74365829 | 74369369 | 161542 |
| 65539 | 74378410 | 74382231 | 161542, 176170 |
| 65540 | 74442600 | 74444560 | 175931 |
| 65541 | 74448407 | 74449468 | 129673, 175931 |
| 65542 | 74492520 | 74492930 | 129667 |
| 65543 | 74493615 | 74497951 | 129667 |
| 65544 | 74520740 | 74521320 | |
| 65545 | 74546284 | 74548438 | 272386, 161544, 267543, |
| | | | 214140, 267546 |
| 65546 | 74552451 | 74555050 | 163597, 267546 |
| 65547 | 74561298 | 74562326 | 163597, 070731 |
| 65548 | 74581758 | 74582572 | 070731 |
| 65549 | 74584129 | 74584927 | |
| 65550 | 74592660 | 74593853 | |
| 65551 | 74721637 | 74723944 | 181038, 070495 |
| 65552 | 74731762 | 74734977 | 092931, 207556, 161547, |
| | | | 267168 |
| 65553 | 74998167 | 74998651 | |
| 65554 | 75084108 | 75085838 | 234912, 129657 |
| 65555 | 75117773 | 75119006 | 129657 |
| 65556 | 75135811 | 75137878 | 129657 |
| 65557 | 75141741 | 75142469 | 129657 |
| 65558 | 75142972 | 75143644 | 129657 |
| 65559 | 75251375 | 75251968 | |
| 65560 | 75276694 | 75279052 | 263718, 184640 |
| 65561 | 75280816 | 75285354 | 184640 |
| 65562 | 75302290 | 75304269 | 184640 |
| 65563 | 75311535 | 75313186 | 184640 |
| 65564 | 75318290 | 75322763 | 184640 |
| 65565 | 75323064 | 75324196 | 184640 |
| 65566 | 75329061 | 75330088 | 184640 |
| 65567 | 75350038 | 75351693 | 184640 |
| 65568 | 75379805 | 75381177 | 184640 |
| 65569 | 75381666 | 75383007 | 184640 |
| 65570 | 75383780 | 75386568 | 184640 |
| 65571 | 75386579 | 75389836 | 184640 |
| 65572 | 75416481 | 75421256 | 184640 |
| 65573 | 75422823 | 75424787 | 184640 |
| 65574 | 75425506 | 75427261 | 184640 |
| 65575 | 75445659 | 75448660 | 184640 |
| 65576 | 75449563 | 75451698 | 184640 |
| 65577 | 75832404 | 75833201 | |
| 65578 | 75953612 | 75955872 | |
| 65579 | 76147567 | 76148021 | 187997 |
| 65580 | 76163964 | 76165816 | 108639 |
| 65581 | 76169935 | 76172038 | 167900 |
| 65582 | 76180327 | 76181597 | 167900 |
| 65583 | 76182548 | 76184687 | 183077, 167900 |
| 65584 | 76210110 | 76210566 | 268310, 089685 |
| 65585 | 76249934 | 76250925 | 267676 |
| 65586 | 76261767 | 76262214 | 204277 |
| 65587 | 76287927 | 76288780 | |
| 65588 | 76309562 | 76313291 | 267737 |
| 65589 | 76326756 | 76327277 | 267737 |
| 65590 | 76337181 | 76339065 | 267737 |
| 65591 | 76347421 | 76350309 | |
| 65592 | 76353200 | 76354132 | 184557 |
| 65593 | 76355040 | 76357345 | 266970, 184557 |
| 65594 | 76371695 | 76372767 | |
| 65595 | 76373989 | 76375845 | 087157 |
| 65596 | 76417507 | 76418543 | 087157 |
| 65597 | 76553672 | 76555387 | 187775 |
| 65598 | 76587834 | 76589148 | |
| 65599 | 76702393 | 76703018 | 108669 |
| 65600 | 76704432 | 76705097 | 108669 |
| 65601 | 76712798 | 76714902 | 108669 |
| 65602 | 76730952 | 76734001 | 108669 |
| 65603 | 76742002 | 76745600 | 108669 |
| 65604 | 76747492 | 76748384 | 108669 |
| 65605 | 76775952 | 76779514 | 108669 |
| 65606 | 76785804 | 76786332 | 055483 |
| 65607 | 76835411 | 76838125 | 055483 |
| 65608 | 76929345 | 76929949 | |
| 65609 | 77004684 | 77006721 | 171302 |
| 65610 | 77022318 | 77024481 | 265096, 173918 |
| 65611 | 77070570 | 77071942 | 167280 |
| 65612 | 77220062 | 77221729 | 167281 |
| 65613 | 77268173 | 77269837 | 167281 |
| 65614 | 77704554 | 77705187 | 182156 |
| 65615 | 77706247 | 77708015 | 182156 |
| 65616 | 77770283 | 77772372 | 141570 |
| 65617 | 77786277 | 77787779 | |
| 65618 | 77805224 | 77807334 | 141582 |
| 65619 | 77809776 | 77814631 | 141582 |
| 65620 | 77817115 | 77819455 | |
| 65621 | 77871354 | 77871963 | |
| 65622 | 77892480 | 77893362 | 262188, 262585 |
| 65623 | 77905007 | 77905985 | |
| 65624 | 77960300 | 77963485 | 167291 |
| 65625 | 77963514 | 77964532 | 167291 |
| 65626 | 77964730 | 77968499 | 262172, 167291 |
| 65627 | 77981958 | 77982737 | 167291 |
| 65628 | 77992985 | 77993965 | 167291 |
| 65629 | 77997320 | 77998060 | 261978, 167291 |
| 65630 | 78002779 | 78005096 | 167291 |
| 65631 | 78008882 | 78011012 | 141519, 167291 |
| 65632 | 78042483 | 78043076 | 141519 |
| 65633 | 78044380 | 78045748 | 141519 |
| 65634 | 78074613 | 78076379 | 171298 |
| 65635 | 78104892 | 78105249 | |
| 65636 | 78120108 | 78122146 | 141543 |
| 65637 | 78169386 | 78170022 | 141527 |
| 65638 | 78193105 | 78194929 | 181045, 181523 |
| 65639 | 78228416 | 78229162 | |
| 65640 | 78233141 | 78236368 | 173821 |
| 65641 | 78236625 | 78238584 | 173821 |
| 65642 | 78388446 | 78389955 | 173818, 263069 |
| 65643 | 78427213 | 78429648 | 260369 |
| 65644 | 78518209 | 78519834 | 141564 |
| 65645 | 78700631 | 78701618 | 141564 |
| 65646 | 78717094 | 78717602 | 141564 |
| 65647 | 78806025 | 78807303 | 141564 |
| 65648 | 78962725 | 78963510 | |
| 65649 | 78964866 | 78966775 | 176108 |
| 65650 | 79008240 | 79010003 | 175866, 226137 |
| 65651 | 79010453 | 79012492 | 175866 |
| 65652 | 79017577 | 79018261 | 175866 |
| 65653 | 79018464 | 79019068 | 175866 |
| 65654 | 79039198 | 79040294 | 175866 |
| 65655 | 79042745 | 79043701 | 175866 |
| 65656 | 79055357 | 79057954 | 175866 |
| 65657 | 79058392 | 79059158 | 175866 |
| 65658 | 79070637 | 79072249 | 175866 |
| 65659 | 79075271 | 79076117 | 175866 |
| 65660 | 79196801 | 79197555 | |
| 65661 | 79211985 | 79214219 | 224877, 167302 |
| 65662 | 79218819 | 79219640 | 157637 |
| 65663 | 79230282 | 79230912 | 157637 |
| 65664 | 79267986 | 79270042 | 157637 |
| 65665 | 79277696 | 79278435 | 185168 |
| 65666 | 79282481 | 79283988 | 185168 |
| 65667 | 79300508 | 79301374 | 185332 |
| 65668 | 79302226 | 79305222 | 185332 |
| 65669 | 79313343 | 79314190 | |
| 65670 | 79314235 | 79320863 | |
| 65671 | 79359738 | 79362110 | 262877 |
| 65672 | 79366399 | 79368233 | |
| 65673 | 79368941 | 79371914 | 263053 |
| 65674 | 79372309 | 79374946 | 266392, 171282, 263053 |
| 65675 | 79396449 | 79397572 | 171282 |
| 65676 | 79451148 | 79451998 | |
| 65677 | 79454706 | 79456094 | |
| 65678 | 79475699 | 79477136 | 184009 |
| 65679 | 79478558 | 79482629 | 229848, 229947, 184009 |
| 65680 | 79486038 | 79487384 | 184009, 229848 |
| 65681 | 79516111 | 79516563 | 185504 |
| 65682 | 79518350 | 79520443 | 185504 |
| 65683 | 79523048 | 79523797 | |
| 65684 | 79533738 | 79534403 | 182446 |
| 65685 | 79562024 | 79562425 | 182446 |
| 65686 | 79602470 | 79604527 | 182612, 182446 |
| 65687 | 79632614 | 79634595 | 185298, 204237 |
| 65688 | 79649998 | 79652506 | 185359, 214087 |
| 65689 | 79669243 | 79671780 | 183048, 262814, 262660, |
| | | | 262049, 185359 |
| 65690 | 79677562 | 79681832 | 183048, 262660 |
| 65691 | 79689072 | 79690128 | |
| 65692 | 79694188 | 79695052 | |
| 65693 | 79700702 | 79701782 | |
| 65694 | 79702086 | 79703532 | |
| 65695 | 79762074 | 79763663 | 215644 |
| 65696 | 79779263 | 79780628 | 225663, 215621 |
| 65697 | 79790079 | 79792091 | 182676, 225663 |
| 65698 | 79798591 | 79799788 | |
| 65699 | 79800447 | 79801597 | 262831, 185624 |
| 65700 | 79812777 | 79813351 | 185624 |
| 65701 | 79816399 | 79820030 | 185624 |
| 65702 | 79820586 | 79821543 | |
| 65703 | 79823013 | 79830611 | 141522, 262413 |
| 65704 | 79842996 | 79844408 | |
| 65705 | 79848143 | 79850413 | 141552, 183684 |
| 65706 | 79860053 | 79861169 | 183979, 185813 |
| 65707 | 79867842 | 79869780 | 185813 |
| 65708 | 79875017 | 79876317 | 197063, 187531 |
| 65709 | 79880691 | 79882297 | 264769, 197063 |
| 65710 | 79882629 | 79883199 | 197063 |
| 65711 | 79883776 | 79886032 | 265688, 197063 |
| 65712 | 79895166 | 79895587 | 183010 |
| 65713 | 79911902 | 79912392 | 185269 |
| 65714 | 79916903 | 79918509 | 185269 |
| 65715 | 79925487 | 79926275 | |
| 65716 | 79934116 | 79937879 | 169696 |
| 65717 | 79948840 | 79950396 | 169696 |
| 65718 | 79958101 | 79959117 | 169696 |
| 65719 | 79979144 | 79980923 | 169689 |
| 65720 | 79983777 | 79984570 | 169683 |
| 65721 | 79988042 | 79989268 | 169683 |
| 65722 | 79993918 | 79996165 | 264569, 169738 |
| 65723 | 79997457 | 79998156 | |
| 65724 | 80008384 | 80011190 | 169727, 169733 |
| 65725 | 80022342 | 80024556 | 169718 |
| 65726 | 80053249 | 80054455 | 169710 |
| 65727 | 80054763 | 80063345 | 176155, 169710 |
| 65728 | 80063572 | 80066957 | 176155 |
| 65729 | 80068032 | 80068827 | 176155 |
| 65730 | 80070110 | 80071259 | 176155 |
| 65731 | 80075559 | 80076106 | 176155 |
| 65732 | 80091240 | 80092391 | 176155 |
| 65733 | 80169969 | 80171738 | 176155 |
| 65734 | 80173298 | 80173799 | 264548 |
| 65735 | 80174228 | 80176338 | 264548 |
| 65736 | 80188572 | 80190120 | 141526 |
| 65737 | 80199388 | 80200399 | 141526, 141551 |
| 65738 | 80223981 | 80227666 | 141551 |
| 65739 | 80230155 | 80231968 | 141551 |
| 65740 | 80249857 | 80251410 | 265692 |
| 65741 | 80253293 | 80253953 | |
| 65742 | 80255010 | 80256993 | |
| 65743 | 80283669 | 80284214 | 141574 |
| 65744 | 80285810 | 80287186 | 141574 |
| 65745 | 80292273 | 80293424 | |
| 65746 | 80328338 | 80328785 | |
| 65747 | 80341889 | 80343882 | |
| 65748 | 80370406 | 80371004 | 181396 |
| 65749 | 80375969 | 80376965 | 169660, 181396 |
| 65750 | 80402180 | 80402789 | 178927 |
| 65751 | 80407936 | 80409240 | 178927 |
| 65752 | 80415315 | 80417385 | 141562, 265458 |
| 65753 | 80440883 | 80441941 | 266236, 141562 |
| 65754 | 80451979 | 80452905 | |
| 65755 | 80454525 | 80456148 | |
| 65756 | 80460096 | 80460571 | |
| 65757 | 80465513 | 80466190 | |
| 65758 | 80475101 | 80475522 | |
| 65759 | 80476674 | 80478875 | 141568 |
| 65760 | 80483248 | 80483890 | 141568 |
| 65761 | 80486435 | 80487083 | 141568 |
| 65762 | 80509590 | 80510129 | 141568 |
| 65763 | 80526160 | 80527013 | 141568 |
| 65764 | 80535753 | 80536212 | 262652, 141568 |
| 65765 | 80605305 | 80606603 | 141580 |
| 65766 | 80642062 | 80643107 | 141542 |
| 65767 | 80644162 | 80646125 | 141542 |
| 65768 | 80655475 | 80656570 | 141542 |
| 65769 | 80656602 | 80657205 | 141542 |
| 65770 | 80674116 | 80675398 | 141560, 263063 |
| 65771 | 80692862 | 80694321 | 167363 |
| 65772 | 80695557 | 80696718 | 167363 |
| 65773 | 80698830 | 80700061 | 167363 |
| 65774 | 80709356 | 80710603 | 141556 |
| 65775 | 80729805 | 80730600 | 141556 |
| 65776 | 80794562 | 80795809 | 141579, 141556 |
| 65777 | 80796180 | 80796767 | 141579, 141556 |
| 65778 | 80846794 | 80847895 | 141556 |
| 65779 | 81008693 | 81010555 | 175711 |
| 65780 | 81036801 | 81037532 | |
| 65781 | 81066645 | 81067614 | |

The locations of ODSCs on Chromosome 18 are shown in Table 60.

TABLE 60
ODSC Locations on Chromosome 18

| SEQ ID NO. | Start | End | Overlapping Gene(s) |
|---|---|---|---|
| 65782 | 108036 | 108579 | |
| 65783 | 108968 | 109526 | 263006 |
| 65784 | 158009 | 159759 | 101557 |
| 65785 | 267057 | 269864 | 263884, 079134 |
| 65786 | 657215 | 658602 | 176890, 263727, 176912 |
| 65787 | 682794 | 683996 | 132199 |
| 65788 | 702604 | 703801 | 132199 |
| 65789 | 711037 | 713451 | 132199 |
| 65790 | 811218 | 813265 | 176105 |
| 65791 | 824012 | 824725 | |
| 65792 | 847822 | 849203 | |
| 65793 | 1269972 | 1270525 | 132204 |
| 65794 | 1391977 | 1392691 | 132204 |
| 65795 | 1513369 | 1514013 | 266602 |
| 65796 | 2570201 | 2571944 | 080986, 101574 |
| 65797 | 2592627 | 2593140 | 080986 |
| 65798 | 2654757 | 2657315 | 101596, 266405 |
| 65799 | 2939189 | 2940278 | 101577 |
| 65800 | 2968497 | 2969060 | 101577, 266397 |
| 65801 | 2969956 | 2971168 | 101577, 266397 |
| 65802 | 2978119 | 2987047 | 266397, 101577 |
| 65803 | 3002576 | 3003460 | 101577 |
| 65804 | 3012148 | 3014569 | 101577 |
| 65805 | 3037193 | 3038252 | |
| 65806 | 3051574 | 3052703 | |
| 65807 | 3060493 | 3061245 | |
| 65808 | 3117137 | 3121000 | 101605 |
| 65809 | 3152646 | 3154587 | 101605 |
| 65810 | 3155461 | 3155983 | 101605 |
| 65811 | 3194766 | 3195784 | 101605, 265399 |
| 65812 | 3207430 | 3208549 | 101605, 265399 |
| 65813 | 3209535 | 3210209 | 101605, 265399 |
| 65814 | 3217835 | 3220383 | 101605, 265399 |
| 65815 | 3233074 | 3234560 | 265399 |
| 65816 | 3244821 | 3253523 | 101608, 272688, 265399 |
| 65817 | 3256020 | 3259684 | 101608, 264235 |
| 65818 | 3260461 | 3264998 | 118680, 264235 |
| 65819 | 3265364 | 3266597 | 118680 |
| 65820 | 3269098 | 3269803 | 118680 |
| 65821 | 3368008 | 3368847 | |
| 65822 | 3384116 | 3385887 | |
| 65823 | 3446686 | 3457479 | 177426 |
| 65824 | 3459166 | 3459930 | |
| 65825 | 3592726 | 3598162 | 177337, 170579 |
| 65826 | 3601791 | 3606740 | 262001, 170579 |
| 65827 | 3618010 | 3618797 | 170579 |
| 65828 | 3621576 | 3626462 | 170579 |
| 65829 | 3634153 | 3634827 | 170579 |
| 65830 | 3648974 | 3651072 | 170579 |
| 65831 | 3652048 | 3653976 | 266401, 170579 |
| 65832 | 3665359 | 3666948 | 170579 |
| 65833 | 3710610 | 3711475 | 170579 |
| 65834 | 3771097 | 3772084 | 266513, 170579 |
| 65835 | 3816207 | 3818150 | 170579 |
| 65836 | 3966294 | 3967843 | 170579, 263878 |
| 65837 | 5236927 | 5239493 | 264575, 265091, 263753 |
| 65838 | 5253747 | 5254400 | |
| 65839 | 5294467 | 5297252 | 198081 |
| 65840 | 5908919 | 5910644 | 264449 |
| 65841 | 5966234 | 5967387 | 154655 |
| 65842 | 5998429 | 5998983 | 154655 |
| 65843 | 6052403 | 6052827 | 154655, 266846 |
| 65844 | 6159160 | 6160833 | 154655 |
| 65845 | 6209941 | 6211781 | 154655 |
| 65846 | 6215672 | 6216274 | 154655 |
| 65847 | 6281748 | 6282313 | 154655 |
| 65848 | 6315518 | 6316331 | 154655 |
| 65849 | 6355115 | 6356923 | 154655 |
| 65850 | 6370316 | 6371899 | 154655 |
| 65851 | 6373738 | 6377351 | 154655 |
| 65852 | 6405213 | 6407760 | 154655 |
| 65853 | 6413178 | 6415930 | 154655 |
| 65854 | 6482253 | 6483228 | |
| 65855 | 6900938 | 6902118 | 088756 |
| 65856 | 7566162 | 7569447 | 173482 |
| 65857 | 7576166 | 7578183 | 173482 |
| 65858 | 7617500 | 7618351 | 173482 |
| 65859 | 7625911 | 7628027 | 173482 |
| 65860 | 7637247 | 7637921 | 173482 |
| 65861 | 7657767 | 7658336 | 173482 |
| 65862 | 7754141 | 7756441 | 266767, 173482 |
| 65863 | 7812343 | 7812969 | 173482 |
| 65864 | 7829537 | 7830100 | 173482 |
| 65865 | 7870884 | 7873978 | 173482 |
| 65866 | 7876520 | 7879986 | 173482 |
| 65867 | 7898957 | 7899569 | 173482 |
| 65868 | 7914631 | 7915661 | 173482 |
| 65869 | 7920448 | 7924423 | 173482 |
| 65870 | 7945307 | 7947237 | 173482 |
| 65871 | 8115279 | 8118538 | 173482 |
| 65872 | 8124239 | 8125896 | 173482 |
| 65873 | 8176471 | 8178002 | 173482 |
| 65874 | 8283885 | 8284403 | 173482 |
| 65875 | 8288843 | 8289322 | 173482 |
| 65876 | 8298844 | 8299539 | 173482 |
| 65877 | 8301209 | 8301899 | 173482 |
| 65878 | 8339164 | 8343267 | 173482 |
| 65879 | 8413096 | 8413858 | 266613 |
| 65880 | 8608629 | 8610893 | 206418 |
| 65881 | 8704284 | 8707067 | 168502, 265962 |
| 65882 | 8707861 | 8708295 | 168502 |
| 65883 | 8730064 | 8730847 | 168502 |
| 65884 | 8799782 | 8801583 | 168502 |
| 65885 | 8858876 | 8859795 | |
| 65886 | 8889740 | 8892220 | |
| 65887 | 8959308 | 8962734 | |
| 65888 | 8989372 | 8991205 | |
| 65889 | 8998312 | 8999727 | |
| 65890 | 9054745 | 9055822 | |
| 65891 | 9067500 | 9068926 | |
| 65892 | 9073207 | 9074835 | |
| 65893 | 9091853 | 9093152 | |
| 65894 | 9093602 | 9095987 | |
| 65895 | 9101544 | 9105425 | 265257, 178127 |
| 65896 | 9116169 | 9119449 | 265257, 178127 |
| 65897 | 9135610 | 9138550 | 101745, 266053, 265257 |
| 65898 | 9153145 | 9153721 | 101745, 265257 |
| 65899 | 9333988 | 9335341 | 128791, 264964 |
| 65900 | 9474225 | 9476569 | 017797 |
| 65901 | 9557246 | 9557893 | 154845 |
| 65902 | 9608648 | 9609334 | 154845 |
| 65903 | 9613392 | 9615998 | 263627, 154845 |
| 65904 | 9643388 | 9644003 | |
| 65905 | 9672611 | 9674191 | |
| 65906 | 9779150 | 9780292 | 168461 |
| 65907 | 9869843 | 9870496 | |
| 65908 | 9898667 | 9899569 | |
| 65909 | 9913362 | 9915855 | 101558 |
| 65910 | 10525636 | 10526975 | 134265 |
| 65911 | 10927048 | 10928242 | 154864 |
| 65912 | 11003680 | 11006048 | 154864 |
| 65913 | 11118463 | 11122829 | 154864 |
| 65914 | 11125525 | 11126072 | 154864 |
| 65915 | 11146495 | 11147309 | 154864 |
| 65916 | 11148697 | 11150261 | |
| 65917 | 11273906 | 11274775 | |
| 65918 | 11283443 | 11284058 | |
| 65919 | 11323289 | 11324008 | |
| 65920 | 11500288 | 11501133 | 267252 |
| 65921 | 11688855 | 11689355 | 141404 |
| 65922 | 11849768 | 11852475 | 267165, 255112, 141404 |
| 65923 | 11856532 | 11858129 | 272703, 141404 |
| 65924 | 11872399 | 11873153 | 141404 |
| 65925 | 11885122 | 11885821 | 141404, 154889 |
| 65926 | 11887514 | 11888243 | 154889 |
| 65927 | 11907699 | 11910102 | 273141, 154889 |
| 65928 | 11946278 | 11948434 | |
| 65929 | 11979787 | 11982457 | 141401 |
| 65930 | 11994331 | 11995123 | 266955, 141401 |
| 65931 | 12008227 | 12008699 | 141401 |
| 65932 | 12015287 | 12016126 | 141401 |
| 65933 | 12017061 | 12017656 | 141401 |
| 65934 | 12026420 | 12027655 | 141401 |
| 65935 | 12038327 | 12039559 | |
| 65936 | 12255656 | 12256019 | 176194 |
| 65937 | 12307998 | 12309153 | 176014 |
| 65938 | 12336841 | 12337298 | 176014, 141385 |
| 65939 | 12375786 | 12378370 | 141385 |
| 65940 | 12657318 | 12658311 | 215527, 134278 |
| 65941 | 12702023 | 12703871 | 101624, 128789 |
| 65942 | 12837053 | 12839286 | 175354 |
| 65943 | 12882498 | 12884774 | 175354 |
| 65944 | 12946454 | 12949056 | 085415 |
| 65945 | 12990814 | 12992896 | 101639, 266969 |
| 65946 | 13136437 | 13139076 | |
| 65947 | 13174861 | 13177718 | |
| 65948 | 13179139 | 13180360 | |
| 65949 | 13215885 | 13219388 | 168675, 267239 |
| 65950 | 13312888 | 13313827 | 168675 |
| 65951 | 13337492 | 13338953 | 168675 |
| 65952 | 13417617 | 13419195 | 168675 |
| 65953 | 13433004 | 13434233 | 168675 |
| 65954 | 13434457 | 13438877 | 168675 |
| 65955 | 13445976 | 13448584 | 168675 |
| 65956 | 13465115 | 13465892 | 267694, 168675 |
| 65957 | 13499888 | 13503282 | 267366, 168675 |
| 65958 | 13512757 | 13513455 | 168675 |
| 65959 | 13539694 | 13542742 | 168675 |
| 65960 | 13725376 | 13727671 | 101654, 177150 |
| 65961 | 18518717 | 18519520 | |
| 65962 | 18632608 | 18634120 | 067900 |
| 65963 | 18690257 | 18692779 | 067900 |
| 65964 | 18763607 | 18764298 | |
| 65965 | 18821147 | 18823750 | 141449, 265984 |
| 65966 | 18844444 | 18845344 | 141449 |
| 65967 | 18909797 | 18910858 | 141449 |
| 65968 | 19018865 | 19021054 | 265751, 141449 |
| 65969 | 19032726 | 19034382 | 141449 |
| 65970 | 19036249 | 19037494 | 141449 |
| 65971 | 19117820 | 19118664 | 141446 |
| 65972 | 19129591 | 19132130 | 141446 |
| 65973 | 19179078 | 19181416 | 141446 |
| 65974 | 19217670 | 19219155 | |
| 65975 | 19248033 | 19250263 | 158201 |
| 65976 | 19251906 | 19254391 | 265656, 158201 |
| 65977 | 19277717 | 19278131 | 158201 |
| 65978 | 19282751 | 19285210 | 101752, 158201 |
| 65979 | 19320331 | 19322568 | 101752 |
| 65980 | 19545577 | 19546105 | |
| 65981 | 19560501 | 19561454 | 265758 |
| 65982 | 19600121 | 19600921 | 265758 |
| 65983 | 19601528 | 19601883 | 265758 |
| 65984 | 19611651 | 19613461 | 265758 |
| 65985 | 19637873 | 19638530 | |
| 65986 | 19664027 | 19665899 | |
| 65987 | 19667581 | 19668892 | 263588 |
| 65988 | 19686200 | 19687230 | |
| 65989 | 19693075 | 19693949 | |
| 65990 | 19744792 | 19751349 | 141448, 266010 |
| 65991 | 19790037 | 19790862 | |
| 65992 | 19802969 | 19804041 | 264825 |
| 65993 | 19865141 | 19866119 | |
| 65994 | 19866305 | 19866994 | |
| 65995 | 19925081 | 19929540 | 273348 |
| 65996 | 19979574 | 19980676 | |
| 65997 | 20002702 | 20005178 | |
| 65998 | 20007670 | 20009772 | |
| 65999 | 20012444 | 20013508 | |
| 66000 | 20045327 | 20049289 | |
| 66001 | 20067935 | 20070978 | |
| 66002 | 20104635 | 20105243 | |
| 66003 | 20121057 | 20122924 | |
| 66004 | 20130471 | 20133069 | |
| 66005 | 20135828 | 20137630 | |
| 66006 | 20138631 | 20140951 | |
| 66007 | 20142760 | 20143966 | |
| 66008 | 20159340 | 20161065 | |
| 66009 | 20164377 | 20166143 | |
| 66010 | 20183978 | 20185798 | |
| 66011 | 20217541 | 20219898 | |
| 66012 | 20253214 | 20253703 | |
| 66013 | 20253869 | 20255428 | |
| 66014 | 20262289 | 20265081 | |
| 66015 | 20283997 | 20284820 | 265943 |
| 66016 | 20289717 | 20292658 | 265943 |
| 66017 | 20411309 | 20412120 | 266850, 101773, 265943 |
| 66018 | 20452750 | 20453726 | 266850, 101773, 265943 |
| 66019 | 20494386 | 20495237 | 101773, 265943 |
| 66020 | 20512427 | 20514174 | 264817, 265943, 101773 |
| 66021 | 20553604 | 20555993 | 101773 |
| 66022 | 20557610 | 20558929 | 101773 |
| 66023 | 20694394 | 20696089 | |
| 66024 | 20713539 | 20717200 | 134508, 222999 |
| 66025 | 20721007 | 20723576 | 134508 |
| 66026 | 20769454 | 20770361 | 134508 |
| 66027 | 20816956 | 20819022 | 134508, 134490 |
| 66028 | 20839448 | 20840918 | 266495, 134508, 134490 |
| 66029 | 20863897 | 20865309 | 134490 |
| 66030 | 20871348 | 20872791 | 134490 |
| 66031 | 20891667 | 20893561 | 134490 |
| 66032 | 20935966 | 20939229 | 134490 |
| 66033 | 20957357 | 20959296 | 134490 |
| 66034 | 20959385 | 20960915 | 134490 |
| 66035 | 20981269 | 20982288 | 134490 |
| 66036 | 21001918 | 21003910 | 134490 |
| 66037 | 21031984 | 21034665 | 101782 |
| 66038 | 21040189 | 21041717 | 101782 |
| 66039 | 21066436 | 21067293 | 101782 |
| 66040 | 21082430 | 21085034 | 141452 |
| 66041 | 21094853 | 21096301 | 141452, 141458 |
| 66042 | 21108910 | 21110694 | 141452, 141458 |
| 66043 | 21126244 | 21126812 | 141458 |
| 66044 | 21164755 | 21167972 | 141458 |
| 66045 | 21197568 | 21200197 | 154065 |
| 66046 | 21205781 | 21208766 | 154065 |
| 66047 | 21209141 | 21210072 | 154065 |
| 66048 | 21241997 | 21243102 | 154065 |
| 66049 | 21264332 | 21264996 | |
| 66050 | 21269078 | 21270471 | 053747 |
| 66051 | 21275111 | 21275946 | 053747 |
| 66052 | 21297698 | 21301228 | 053747 |
| 66053 | 21404410 | 21405340 | 053747 |
| 66054 | 21427166 | 21427887 | 053747 |
| 66055 | 21437113 | 21438345 | 053747 |
| 66056 | 21439313 | 21440349 | 053747 |
| 66057 | 21462978 | 21464295 | 053747 |
| 66058 | 21561818 | 21563002 | 265752 |
| 66059 | 21593671 | 21596143 | 264745, 168234 |
| 66060 | 21630630 | 21632753 | 168234 |
| 66061 | 21643004 | 21643464 | 168234 |
| 66062 | 21691679 | 21696125 | 264924, 168234 |
| 66063 | 21718578 | 21719540 | 154040 |
| 66064 | 21775249 | 21775951 | 141447 |
| 66065 | 21791507 | 21792224 | 141447 |
| 66066 | 21811235 | 21812616 | 141447 |
| 66067 | 21851181 | 21852451 | 141447 |
| 66068 | 21977040 | 21978332 | 141447 |
| 66069 | 22005926 | 22008157 | 154059 |
| 66070 | 22126030 | 22126725 | |
| 66071 | 23669476 | 23671600 | 141380 |
| 66072 | 23965940 | 23966406 | 141384 |
| 66073 | 24127761 | 24129498 | 134504 |
| 66074 | 24155216 | 24155880 | 134504 |
| 66075 | 24159765 | 24160533 | 134504 |
| 66076 | 24235583 | 24237684 | 260372, 134504 |
| 66077 | 24322160 | 24323040 | 260372 |
| 66078 | 24324953 | 24328050 | 260372 |
| 66079 | 24332223 | 24332945 | 260372 |
| 66080 | 24333567 | 24334822 | 260372 |
| 66081 | 24336536 | 24338643 | 260372 |
| 66082 | 24372549 | 24373111 | 260372 |
| 66083 | 24443100 | 24443731 | 260372, 171885 |
| 66084 | 24454880 | 24456045 | 260372 |
| 66085 | 24637276 | 24638288 | 260372, 154080 |
| 66086 | 24715359 | 24717433 | 260372, 154080 |
| 66087 | 24721710 | 24724989 | 260372, 154080 |
| 66088 | 24764990 | 24765960 | 154080, 260372 |
| 66089 | 24813070 | 24813551 | 265374 |
| 66090 | 24958183 | 24958871 | 264151 |
| 66091 | 25201005 | 25201837 | |
| 66092 | 25236057 | 25236843 | |
| 66093 | 25241202 | 25242049 | |
| 66094 | 25329884 | 25330505 | |
| 66095 | 25576771 | 25577168 | 170558 |
| 66096 | 25599515 | 25600276 | 170558 |
| 66097 | 25653112 | 25654302 | 170558 |
| 66098 | 25666739 | 25667875 | 170558 |
| 66099 | 25670670 | 25672177 | 170558 |
| 66100 | 25690899 | 25696940 | 170558 |
| 66101 | 25706958 | 25708688 | 170558 |
| 66102 | 25743131 | 25743540 | 170558 |
| 66103 | 25747633 | 25748218 | 170558 |
| 66104 | 25753127 | 25759140 | 170558 |
| 66105 | 25763026 | 25764536 | |
| 66106 | 26743319 | 26745774 | |
| 66107 | 28591133 | 28592065 | 134762 |
| 66108 | 28622276 | 28622873 | 134762 |
| 66109 | 28680478 | 28682972 | 265888, 134755 |
| 66110 | 28813601 | 28814492 | |
| 66111 | 28821776 | 28822898 | |
| 66112 | 28823039 | 28825436 | |
| 66113 | 28827196 | 28828308 | |
| 66114 | 28847521 | 28849944 | |
| 66115 | 28854710 | 28855984 | |
| 66116 | 28882770 | 28883451 | |
| 66117 | 28904671 | 28905563 | 134760 |
| 66118 | 28979502 | 28982707 | 175065, 266729 |
| 66119 | 29077250 | 29079485 | 046604 |
| 66120 | 29085972 | 29086821 | 046604 |
| 66121 | 29091743 | 29093956 | 046604 |
| 66122 | 29161318 | 29162157 | |
| 66123 | 29171135 | 29173185 | 118271 |
| 66124 | 29258361 | 29260811 | 118276 |
| 66125 | 29264184 | 29265601 | 118276 |
| 66126 | 29303625 | 29304953 | 265158 |
| 66127 | 29318056 | 29320857 | |
| 66128 | 29353817 | 29354957 | |
| 66129 | 29372241 | 29373198 | |
| 66130 | 29521574 | 29524271 | 263823, 153339 |
| 66131 | 29534105 | 29534961 | |
| 66132 | 29562818 | 29563920 | |
| 66133 | 29598016 | 29600161 | 101695, 263917 |
| 66134 | 29671410 | 29673953 | 134758, 265008, 263917 |
| 66135 | 29675694 | 29676395 | 134758, 263917 |
| 66136 | 29787885 | 29788416 | 141441, 141434 |
| 66137 | 29793482 | 29794132 | 141441, 141434 |
| 66138 | 29858042 | 29859066 | 141441 |
| 66139 | 29881285 | 29882689 | 141441 |
| 66140 | 29897134 | 29898384 | 141441 |
| 66141 | 29929011 | 29931620 | 141441 |
| 66142 | 29938510 | 29939059 | 141441 |
| 66143 | 29945843 | 29948578 | 141441 |
| 66144 | 29951094 | 29954377 | 141441 |
| 66145 | 29957646 | 29959677 | 141441 |
| 66146 | 29962079 | 29964275 | 141441 |
| 66147 | 30006942 | 30007701 | 141441 |
| 66148 | 30049027 | 30051889 | 141441 |
| 66149 | 31157649 | 31158251 | 267642 |
| 66150 | 31158386 | 31159265 | 141431 |
| 66151 | 31335905 | 31336812 | |
| 66152 | 31400540 | 31402202 | |
| 66153 | 31446283 | 31447145 | 101746 |
| 66154 | 31449692 | 31452322 | 101746 |
| 66155 | 31608141 | 31608820 | 101746 |
| 66156 | 31618917 | 31619816 | 101746 |
| 66157 | 31701034 | 31701985 | 101746 |
| 66158 | 32072532 | 32074752 | 134769 |
| 66159 | 32094060 | 32096155 | 134769 |
| 66160 | 32206592 | 32207218 | 134769 |
| 66161 | 32239550 | 32240172 | 134769 |
| 66162 | 32306068 | 32306856 | 134769 |
| 66163 | 32507886 | 32510491 | |
| 66164 | 32527350 | 32528478 | |
| 66165 | 32556515 | 32559809 | 166974 |
| 66166 | 32620472 | 32622075 | 166974 |
| 66167 | 32639922 | 32640776 | 166974 |
| 66168 | 32642313 | 32642847 | 166974 |
| 66169 | 32664516 | 32665198 | 166974 |
| 66170 | 32810879 | 32811468 | |
| 66171 | 32819964 | 32822085 | 186812 |
| 66172 | 32869241 | 32871130 | 257267, 186814, 268573 |
| 66173 | 32923351 | 32926036 | 172466 |
| 66174 | 32956459 | 32957463 | 186496 |
| 66175 | 33037926 | 33039548 | 267583, 267140, 153391 |
| 66176 | 33057320 | 33063285 | 267140, 153391 |
| 66177 | 33076666 | 33078425 | 267140, 153391 |
| 66178 | 33160490 | 33162684 | 141429 |
| 66179 | 33551604 | 33553433 | 141428 |
| 66180 | 33625496 | 33626038 | 141425 |
| 66181 | 33646475 | 33647973 | 141425 |
| 66182 | 33708506 | 33710778 | 134759, 141424 |
| 66183 | 33732318 | 33734374 | 134759 |
| 66184 | 33735619 | 33738564 | 134759 |
| 66185 | 33755329 | 33756801 | 134759 |
| 66186 | 33766540 | 33769437 | 075643, 260552 |
| 66187 | 33877093 | 33877702 | 134775 |
| 66188 | 34407671 | 34409787 | 150477, 134779 |
| 66189 | 34879422 | 34880040 | 101489 |
| 66190 | 34943832 | 34944598 | 101489 |
| 66191 | 36520011 | 36522238 | |
| 66192 | 36531071 | 36532216 | |
| 66193 | 36560329 | 36560769 | |
| 66194 | 36986834 | 36987526 | 267374 |
| 66195 | 37102969 | 37104031 | 267374 |
| 66196 | 37525909 | 37527618 | |
| 66197 | 39159026 | 39159635 | 267313 |
| 66198 | 39674769 | 39675249 | |
| 66199 | 39750936 | 39751834 | 267586 |
| 66200 | 39772030 | 39773181 | 267586 |
| 66201 | 39918196 | 39921247 | 267586 |
| 66202 | 39991604 | 39998430 | 267586 |
| 66203 | 40006201 | 40007044 | 267586 |
| 66204 | 40024038 | 40027512 | 267586 |
| 66205 | 40064417 | 40065127 | 267586 |
| 66206 | 40097256 | 40098958 | 267586 |
| 66207 | 40104952 | 40106812 | 267586 |
| 66208 | 40158096 | 40159679 | 267586 |
| 66209 | 40162456 | 40164036 | 267586 |
| 66210 | 40215429 | 40216187 | 267586 |
| 66211 | 40818254 | 40818947 | |
| 66212 | 42231329 | 42231869 | |
| 66213 | 42258737 | 42261645 | 152217, 267414 |
| 66214 | 42276477 | 42277932 | 152217 |
| 66215 | 42287787 | 42290275 | 152217 |
| 66216 | 42304909 | 42306661 | 152217 |
| 66217 | 42312186 | 42314720 | 152217 |
| 66218 | 42351552 | 42352795 | 152217 |
| 66219 | 42368907 | 42370245 | 152217 |
| 66220 | 42378427 | 42379219 | 152217 |
| 66221 | 42387377 | 42388359 | 152217 |
| 66222 | 42399689 | 42403210 | 152217 |
| 66223 | 42432615 | 42433461 | 152217 |
| 66224 | 42464320 | 42464880 | 152217 |
| 66225 | 42465062 | 42465923 | 152217 |
| 66226 | 42466855 | 42468255 | 152217 |
| 66227 | 42495788 | 42498831 | 152217 |
| 66228 | 42506305 | 42508128 | 152217 |
| 66229 | 42517681 | 42520307 | 152217 |
| 66230 | 42640981 | 42641858 | 152217 |
| 66231 | 42748829 | 42750017 | 267101 |
| 66232 | 42853922 | 42854461 | 132874 |
| 66233 | 42900151 | 42901141 | 132874 |
| 66234 | 42902177 | 42902764 | 132874 |
| 66235 | 42904489 | 42905349 | 132874 |
| 66236 | 42964132 | 42964898 | 132874 |
| 66237 | 43231217 | 43231590 | 132874 |
| 66238 | 43355037 | 43355937 | |
| 66239 | 43372502 | 43374201 | |
| 66240 | 43381524 | 43384139 | |
| 66241 | 43418489 | 43419873 | 197046 |
| 66242 | 43436442 | 43436920 | 152223 |
| 66243 | 43515503 | 43516144 | 152223 |
| 66244 | 43530950 | 43531833 | 152223 |
| 66245 | 43541819 | 43543629 | 152223 |
| 66246 | 43546324 | 43547733 | 152223 |
| 66247 | 43553957 | 43554704 | |
| 66248 | 43607976 | 43608780 | 267293, 152229 |
| 66249 | 43631230 | 43633084 | 152229 |
| 66250 | 43648658 | 43650634 | 152229 |
| 66251 | 43650980 | 43653045 | 152229 |
| 66252 | 43677159 | 43679393 | 152234 |
| 66253 | 43683986 | 43685232 | 152240, 152234 |
| 66254 | 43753157 | 43755098 | 152242 |
| 66255 | 43772861 | 43773475 | 152242 |
| 66256 | 43779412 | 43779941 | 152242 |
| 66257 | 43913328 | 43914111 | 141622 |
| 66258 | 43914290 | 43914758 | 141622 |
| 66259 | 43923251 | 43924394 | 141622 |
| 66260 | 43998585 | 43999866 | 141622 |
| 66261 | 44496680 | 44498153 | 167216, 078043 |
| 66262 | 44675820 | 44678056 | 167220, 267228 |
| 66263 | 44701946 | 44703053 | 267228, 134049 |
| 66264 | 45298791 | 45299372 | |
| 66265 | 45456047 | 45458328 | 175387 |
| 66266 | 45528851 | 45530583 | |
| 66267 | 45534416 | 45535209 | |
| 66268 | 45657477 | 45657928 | 184828 |
| 66269 | 45662482 | 45663670 | 184828 |
| 66270 | 45678863 | 45681492 | 184828 |
| 66271 | 45687106 | 45688746 | 184828 |
| 66272 | 45710038 | 45711014 | 184828 |
| 66273 | 45718552 | 45719691 | 184828 |
| 66274 | 45938522 | 45939237 | |
| 66275 | 46014498 | 46015981 | |
| 66276 | 46064759 | 46068452 | 134030 |
| 66277 | 46073900 | 46075692 | 134030 |
| 66278 | 46078537 | 46078964 | 134030 |
| 66279 | 46079368 | 46080016 | 134030 |
| 66280 | 46080691 | 46082236 | 134030 |
| 66281 | 46089409 | 46089857 | 134030 |
| 66282 | 46092443 | 46094502 | 134030 |
| 66283 | 46101081 | 46103757 | 134030 |
| 66284 | 46107112 | 46108402 | 134030 |
| 66285 | 46116206 | 46118069 | 134030 |
| 66286 | 46161247 | 46161797 | 134030 |
| 66287 | 46193607 | 46194676 | 134030 |
| 66288 | 46201533 | 46203610 | 134030, 267762 |
| 66289 | 46307045 | 46309478 | 134030 |
| 66290 | 46326804 | 46329394 | 134030 |
| 66291 | 46452676 | 46453451 | 101665 |
| 66292 | 46455124 | 46457644 | 101665 |
| 66293 | 46467152 | 46471469 | 101665 |
| 66294 | 46472167 | 46479957 | 101665 |
| 66295 | 46501350 | 46503380 | |
| 66296 | 46516942 | 46517704 | |
| 66297 | 46598390 | 46599841 | 141627 |
| 66298 | 46780965 | 46781675 | 141627 |
| 66299 | 46985719 | 46987736 | 141627 |
| 66300 | 47012534 | 47014076 | 265496, 177576, 215472 |
| 66301 | 47017765 | 47019149 | 271982, 265681, 215472, |
| | | | 265496 |
| 66302 | 47040659 | 47041543 | |
| 66303 | 47074577 | 47074943 | |
| 66304 | 47086750 | 47091306 | 101670 |
| 66305 | 47135633 | 47136671 | |
| 66306 | 47151411 | 47152317 | |
| 66307 | 47154506 | 47158377 | |
| 66308 | 47164267 | 47167953 | |
| 66309 | 47176696 | 47178429 | 267444 |
| 66310 | 47208537 | 47209431 | |
| 66311 | 47211522 | 47212121 | |
| 66312 | 47254336 | 47255309 | |
| 66313 | 47283230 | 47283914 | |
| 66314 | 47328953 | 47329476 | 167315, 266997 |
| 66315 | 47338267 | 47341531 | 267322, 251992, 252139, |
| | | | 167315, 266997 |
| 66316 | 47358358 | 47366284 | 266997, 267322, 167306 |
| 66317 | 47492460 | 47494013 | 167306 |
| 66318 | 47519659 | 47521776 | 167306 |
| 66319 | 47550590 | 47551423 | 167306 |
| 66320 | 47602126 | 47602589 | 167306 |
| 66321 | 47605844 | 47606717 | 167306 |
| 66322 | 47625609 | 47627877 | 167306 |
| 66323 | 47641581 | 47642040 | 167306 |
| 66324 | 47653948 | 47655692 | 167306 |
| 66325 | 47680470 | 47681372 | 167306 |
| 66326 | 47719915 | 47721910 | 167306 |
| 66327 | 47794178 | 47795196 | 141644 |
| 66328 | 47806732 | 47808846 | 154832, 141644 |
| 66329 | 47813260 | 47815064 | 154832 |
| 66330 | 47834041 | 47835431 | 267695 |
| 66331 | 47837712 | 47838750 | 267695 |
| 66332 | 47900450 | 47901535 | 154839 |
| 66333 | 47937005 | 47937929 | |
| 66334 | 47951407 | 47952071 | |
| 66335 | 47984796 | 47985705 | 239780 |
| 66336 | 47995171 | 47996091 | |
| 66337 | 48086081 | 48086492 | 141639 |
| 66338 | 48087011 | 48087743 | 141639 |
| 66339 | 48141081 | 48141610 | 141639 |
| 66340 | 48345434 | 48346893 | 134042 |
| 66341 | 48362792 | 48363400 | |
| 66342 | 48379456 | 48381620 | |
| 66343 | 48404528 | 48406447 | 082212 |
| 66344 | 48493655 | 48495735 | 267699, 141646, 141642 |
| 66345 | 48498634 | 48500860 | 267699, 141646, 141642 |
| 66346 | 48512707 | 48513541 | 267699, 141646, 141642 |
| 66347 | 48555668 | 48558060 | 267699, 141646 |
| 66348 | 48563791 | 48566721 | 267699, 141646 |
| 66349 | 48660023 | 48660659 | |
| 66350 | 48676290 | 48677320 | |
| 66351 | 48721378 | 48724933 | 176624 |
| 66352 | 49417137 | 49417739 | |
| 66353 | 49434052 | 49435299 | |
| 66354 | 51322221 | 51322955 | |
| 66355 | 51517469 | 51518018 | |
| 66356 | 51674863 | 51676904 | |
| 66357 | 51749343 | 51751501 | 134046 |
| 66358 | 51780271 | 51781653 | |
| 66359 | 51795307 | 51797097 | 101751 |
| 66360 | 51914629 | 51915520 | |
| 66361 | 52274499 | 52275980 | |
| 66362 | 52280046 | 52281221 | |
| 66363 | 52433300 | 52435406 | 041353 |
| 66364 | 52442762 | 52444850 | 041353 |
| 66365 | 52468514 | 52470264 | 041353 |
| 66366 | 52495507 | 52496158 | 041353 |
| 66367 | 52540838 | 52541677 | 041353 |
| 66368 | 52612927 | 52614273 | 166510 |
| 66369 | 52625943 | 52627016 | 166510 |
| 66370 | 53695615 | 53696232 | 206129 |
| 66371 | 53696997 | 53697789 | 206129 |
| 66372 | 54069570 | 54070748 | |
| 66373 | 54238095 | 54238676 | |
| 66374 | 54282945 | 54283425 | 091164 |
| 66375 | 54304655 | 54306806 | 091164 |
| 66376 | 54318042 | 54319475 | 091157, 091164 |
| 66377 | 54557194 | 54559277 | 091157 |
| 66378 | 54653909 | 54654883 | 091157 |
| 66379 | 54717542 | 54718510 | |
| 66380 | 54799011 | 54799745 | |
| 66381 | 54808145 | 54810086 | 267789 |
| 66382 | 54877457 | 54878639 | |
| 66383 | 55089443 | 55090146 | |
| 66384 | 55094080 | 55098804 | |
| 66385 | 55099530 | 55106418 | 119547, 266636 |
| 66386 | 55112496 | 55114735 | 119547 |
| 66387 | 55136928 | 55137884 | 119547 |
| 66388 | 55200194 | 55202450 | |
| 66389 | 55252937 | 55254618 | 066926 |
| 66390 | 55288140 | 55289518 | 134440 |
| 66391 | 55296908 | 55298438 | 267040 |
| 66392 | 55360645 | 55362089 | 267787, 267040, 081923 |
| 66393 | 55379249 | 55380390 | 267787, 267040, 081923 |
| 66394 | 55404708 | 55406206 | 267787, 267040, 081923 |
| 66395 | 55408432 | 55410291 | 081923 |
| 66396 | 55447434 | 55448650 | 081923 |
| 66397 | 55455318 | 55457866 | 081923 |
| 66398 | 55464179 | 55464918 | 081923 |
| 66399 | 55469119 | 55472778 | 081923 |
| 66400 | 55473537 | 55476498 | |
| 66401 | 55478294 | 55482089 | |
| 66402 | 55486414 | 55488247 | |
| 66403 | 55500173 | 55501491 | |
| 66404 | 55513340 | 55513858 | |
| 66405 | 55567947 | 55570578 | |
| 66406 | 55592279 | 55593914 | |
| 66407 | 55594709 | 55595379 | |
| 66408 | 55641991 | 55642747 | |
| 66409 | 55680500 | 55681398 | |
| 66410 | 55710418 | 55714983 | 049759 |
| 66411 | 55721514 | 55721917 | 049759 |
| 66412 | 55724412 | 55725136 | 049759 |
| 66413 | 55753955 | 55756595 | 049759 |
| 66414 | 55757856 | 55758999 | 049759 |
| 66415 | 55763957 | 55764996 | 049759 |
| 66416 | 55770063 | 55771613 | 049759 |
| 66417 | 55784570 | 55786733 | 049759 |
| 66418 | 55795814 | 55796517 | 049759 |
| 66419 | 55888492 | 55894327 | 049759 |
| 66420 | 55897390 | 55898810 | 049759 |
| 66421 | 55898973 | 55900456 | 049759 |
| 66422 | 55901880 | 55902938 | 049759 |
| 66423 | 55906909 | 55907753 | 049759 |
| 66424 | 55951835 | 55955940 | 049759 |
| 66425 | 55980615 | 55983539 | 049759 |
| 66426 | 56028414 | 56032752 | 049759 |
| 66427 | 56065886 | 56066855 | 267396, 049759 |
| 66428 | 56073882 | 56077508 | |
| 66429 | 56079860 | 56086303 | 267675 |
| 66430 | 56110397 | 56123421 | 267391, 207778 |
| 66431 | 56193294 | 56195239 | 198796 |
| 66432 | 56205548 | 56206775 | 267257, 198796 |
| 66433 | 56207262 | 56210228 | 198796 |
| 66434 | 56217040 | 56217627 | 198796 |
| 66435 | 56218716 | 56220510 | 198796 |
| 66436 | 56233496 | 56238334 | 267579, 198796 |
| 66437 | 56243878 | 56247938 | 198796 |
| 66438 | 56259630 | 56260402 | 198796 |
| 66439 | 56267456 | 56270053 | 252284, 198796 |
| 66440 | 56338249 | 56339801 | 172175, 267226 |
| 66441 | 56349612 | 56350386 | 172175, 267476 |
| 66442 | 56391040 | 56391514 | 172175, 267476 |
| 66443 | 56451462 | 56453703 | |
| 66444 | 56455613 | 56457253 | |
| 66445 | 56529881 | 56531620 | 074657 |
| 66446 | 56619996 | 56620836 | 074657 |
| 66447 | 56658902 | 56661665 | |
| 66448 | 56689422 | 56690269 | 224367 |
| 66449 | 56806496 | 56808051 | 166562 |
| 66450 | 57025188 | 57026947 | 074695 |
| 66451 | 57182985 | 57184740 | 183287 |
| 66452 | 57548939 | 57549904 | |
| 66453 | 57566361 | 57567217 | 141682 |
| 66454 | 59316073 | 59316711 | 267175 |
| 66455 | 59324170 | 59324810 | 267175 |
| 66456 | 59357319 | 59358379 | 267175 |
| 66457 | 59399810 | 59402320 | 267175 |
| 66458 | 59402521 | 59403844 | 267175 |
| 66459 | 59405113 | 59406689 | 267175 |
| 66460 | 59479986 | 59480731 | 176641 |
| 66461 | 59490025 | 59490599 | 176641 |
| 66462 | 59521819 | 59522271 | 176641 |
| 66463 | 59532681 | 59533114 | 176641 |
| 66464 | 59559838 | 59562514 | 176641 |
| 66465 | 59563879 | 59564940 | |
| 66466 | 59565397 | 59566863 | |
| 66467 | 59626618 | 59627818 | |
| 66468 | 59658609 | 59661114 | |
| 66469 | 59740982 | 59741849 | 197563 |
| 66470 | 59853256 | 59855567 | 134444, 197563 |
| 66471 | 60087199 | 60088468 | |
| 66472 | 60092142 | 60093860 | |
| 66473 | 60103625 | 60106619 | |
| 66474 | 60117302 | 60120357 | |
| 66475 | 60127693 | 60128919 | |
| 66476 | 60153413 | 60154104 | |
| 66477 | 60172794 | 60174573 | |
| 66478 | 60176821 | 60177750 | |
| 66479 | 60188179 | 60190721 | 141664 |
| 66480 | 60190737 | 60194968 | 141664 |
| 66481 | 60206035 | 60207760 | 141664 |
| 66482 | 60299980 | 60300614 | |
| 66483 | 60301815 | 60302546 | |
| 66484 | 60381782 | 60385404 | 081913 |
| 66485 | 60408250 | 60409712 | 081913 |
| 66486 | 60433974 | 60435690 | 081913 |
| 66487 | 60445055 | 60450317 | 081913 |
| 66488 | 60463395 | 60464099 | 081913 |
| 66489 | 60467717 | 60468303 | 081913 |
| 66490 | 60478701 | 60479933 | 081913 |
| 66491 | 60506969 | 60508563 | 081913 |
| 66492 | 60666316 | 60667381 | |
| 66493 | 60702907 | 60703376 | |
| 66494 | 60767217 | 60767691 | |
| 66495 | 60814955 | 60816959 | 171791 |
| 66496 | 60822379 | 60823142 | 171791 |
| 66497 | 60828459 | 60829611 | 171791 |
| 66498 | 60904383 | 60906357 | 171791 |
| 66499 | 60908512 | 60909458 | 171791 |
| 66500 | 60986452 | 60988553 | 171791 |
| 66501 | 61002611 | 61004351 | 119537 |
| 66502 | 61016033 | 61016319 | 119537 |
| 66503 | 61032925 | 61035409 | 267390, 119537 |
| 66504 | 61073235 | 61073616 | 119541 |
| 66505 | 61074793 | 61075627 | 119541 |
| 66506 | 61077942 | 61078472 | 119541 |
| 66507 | 61088719 | 61090787 | 119541 |
| 66508 | 61546949 | 61547406 | 197632 |
| 66509 | 61631931 | 61632672 | 221887 |
| 66510 | 61636322 | 61638744 | 166401, 221887 |
| 66511 | 61641959 | 61642681 | 221887, 166401 |
| 66512 | 61648887 | 61649699 | 221887, 166401 |
| 66513 | 61921077 | 61923131 | 267134, 266952 |
| 66514 | 61939197 | 61940363 | 267134 |
| 66515 | 61954658 | 61956674 | 267134 |
| 66516 | 63601948 | 63602559 | |
| 66517 | 63608273 | 63609867 | |
| 66518 | 64391734 | 64392378 | |
| 66519 | 64584030 | 64584865 | |
| 66520 | 64771771 | 64772683 | |
| 66521 | 64800144 | 64800550 | |
| 66522 | 64812652 | 64813550 | |
| 66523 | 65183011 | 65184580 | 265533, 171451 |
| 66524 | 65192431 | 65192949 | 265533 |
| 66525 | 65266264 | 65268130 | 265533 |
| 66526 | 65288089 | 65289332 | 265533 |
| 66527 | 66381218 | 66382888 | 150636, 166479 |
| 66528 | 66651315 | 66652165 | 150636 |
| 66529 | 66675728 | 66676422 | 150636 |
| 66530 | 66738878 | 66739470 | |
| 66531 | 67067358 | 67068455 | 206052 |
| 66532 | 67848201 | 67848761 | 176225 |
| 66533 | 67872232 | 67873624 | 176225 |
| 66534 | 67947284 | 67948276 | |
| 66535 | 67949802 | 67950492 | |
| 66536 | 67955333 | 67958499 | 170677 |
| 66537 | 67989995 | 67990585 | 170677 |
| 66538 | 67999521 | 68005558 | 266304, 266258 |
| 66539 | 68030597 | 68031987 | |
| 66540 | 68043196 | 68045896 | |
| 66541 | 68053565 | 68055428 | |
| 66542 | 68086315 | 68088517 | |
| 66543 | 68088570 | 68091203 | |
| 66544 | 68097736 | 68099266 | 214255 |
| 66545 | 68131866 | 68132494 | |
| 66546 | 68156510 | 68158097 | |
| 66547 | 69665527 | 69666271 | |
| 66548 | 69782096 | 69782992 | |
| 66549 | 70985766 | 70986945 | |
| 66550 | 71007019 | 71008858 | 261780 |
| 66551 | 71814429 | 71816671 | 075336, 141665 |
| 66552 | 71863131 | 71864705 | |
| 66553 | 71864812 | 71865653 | |
| 66554 | 71883766 | 71885502 | 260569 |
| 66555 | 71886212 | 71887562 | 260569 |
| 66556 | 71892170 | 71894573 | 260569 |
| 66557 | 71895015 | 71899677 | 243856, 260569 |
| 66558 | 71899933 | 71903497 | 260569 |
| 66559 | 71907453 | 71910956 | 260569 |
| 66560 | 71914911 | 71918029 | |
| 66561 | 71918389 | 71920171 | |
| 66562 | 71932391 | 71936245 | 166347 |
| 66563 | 71945149 | 71945966 | 166347 |
| 66564 | 71948687 | 71951373 | 166347 |
| 66565 | 71953321 | 71960113 | 166347 |
| 66566 | 71966893 | 71967709 | |
| 66567 | 71983828 | 71985066 | 206043 |
| 66568 | 72034910 | 72035653 | |
| 66569 | 72035699 | 72037013 | |
| 66570 | 72146763 | 72148703 | |
| 66571 | 72162513 | 72164771 | 133313 |
| 66572 | 72165627 | 72167746 | 133313 |
| 66573 | 72180823 | 72181334 | 133313 |
| 66574 | 72183201 | 72185045 | 133313 |
| 66575 | 72208501 | 72210464 | 150656 |
| 66576 | 72263530 | 72266463 | 215421, 264247 |
| 66577 | 72418596 | 72419505 | 215421 |
| 66578 | 72427600 | 72428808 | 215421 |
| 66579 | 72508745 | 72510857 | 215421 |
| 66580 | 72514951 | 72517086 | 215421 |
| 66581 | 72517881 | 72519782 | 215421 |
| 66582 | 72551798 | 72553566 | 215421 |
| 66583 | 72662993 | 72663634 | 215421 |
| 66584 | 72919746 | 72922412 | 180011 |
| 66585 | 72924405 | 72924781 | 179981 |
| 66586 | 72925505 | 72926040 | 179981 |
| 66587 | 72926371 | 72928141 | 179981 |
| 66588 | 72938264 | 72939460 | 179981 |
| 66589 | 72953634 | 72954437 | 179981 |
| 66590 | 72958323 | 72960766 | 179981 |
| 66591 | 74060263 | 74061590 | |
| 66592 | 74171121 | 74173033 | 101493 |
| 66593 | 74187697 | 74190492 | 101493 |
| 66594 | 74202705 | 74208791 | 220032, 265778, 101493 |
| 66595 | 74275532 | 74277876 | 263812 |
| 66596 | 74284509 | 74286834 | 263812 |
| 66597 | 74328328 | 74328713 | 266256 |
| 66598 | 74330938 | 74332342 | 266256 |
| 66599 | 74333037 | 74334686 | 266256 |
| 66600 | 74532945 | 74535630 | 130856, 264278 |
| 66601 | 74556153 | 74558006 | 130856 |
| 66602 | 74647846 | 74648339 | 130856 |
| 66603 | 74665488 | 74665960 | 130856 |
| 66604 | 74667585 | 74668330 | 130856 |
| 66605 | 74693604 | 74694159 | 266844, 197971 |
| 66606 | 74766546 | 74768380 | 197971 |
| 66607 | 74795982 | 74797295 | 197971 |
| 66608 | 74815486 | 74816768 | 197971 |
| 66609 | 74825483 | 74828061 | 197971 |
| 66610 | 74838792 | 74840987 | 197971 |
| 66611 | 74842811 | 74845545 | 197971 |
| 66612 | 74919421 | 74919985 | 265844 |
| 66613 | 75033662 | 75034829 | |
| 66614 | 76828299 | 76830203 | 166377 |
| 66615 | 76915982 | 76917275 | 166377 |
| 66616 | 76964834 | 76965688 | 166377 |
| 66617 | 77012863 | 77013841 | 267628, 166377 |
| 66618 | 77153361 | 77155480 | |
| 66619 | 77159793 | 77160783 | 131196 |
| 66620 | 77267364 | 77268382 | 131196 |
| 66621 | 77437951 | 77440698 | 060069, 178412 |
| 66622 | 77693728 | 77695527 | 122490 |
| 66623 | 77696478 | 77697575 | 122490 |
| 66624 | 77700139 | 77703274 | 122490 |
| 66625 | 77707449 | 77708815 | 122490 |
| 66626 | 77710632 | 77712789 | 122490 |
| 66627 | 77724233 | 77725974 | 226742 |
| 66628 | 77747357 | 77749705 | 141759 |
| 66629 | 77770064 | 77771031 | 141759 |
| 66630 | 77792996 | 77794934 | 267127, 101546, 141759 |
| 66631 | 77866607 | 77868165 | 101544 |

The locations of ODSCs on Chromosome 19 are shown in Table 61.

TABLE 61
ODSC Locations on Chromosome 19

| SEQ ID NO. | Start | End | Overlapping Gene(s) |
|---|---|---|---|
| 66632 | 266938 | 267697 | |
| 66633 | 344195 | 345079 | 105556 |
| 66634 | 409944 | 410682 | |
| 66635 | 459720 | 462465 | 129946 |
| 66636 | 488196 | 490251 | 266933, 099866 |
| 66637 | 506162 | 508134 | 141933, 266933 |
| 66638 | 530430 | 534404 | 099804 |
| 66639 | 534860 | 536303 | 099804 |
| 66640 | 555753 | 556309 | |
| 66641 | 571039 | 574090 | 172270, 267751 |
| 66642 | 584191 | 584942 | |
| 66643 | 632252 | 633843 | 099821 |
| 66644 | 659012 | 664141 | 267666, 070423 |
| 66645 | 674070 | 682156 | 070404 |
| 66646 | 778795 | 779833 | |
| 66647 | 781916 | 783464 | 267530 |
| 66648 | 784382 | 784947 | 267530 |
| 66649 | 796323 | 799915 | 011304 |
| 66650 | 892026 | 893880 | 207507, 175221 |
| 66651 | 912381 | 914000 | 198858 |
| 66652 | 924628 | 927751 | 116017 |
| 66653 | 934271 | 935407 | 116017 |
| 66654 | 940121 | 941779 | 116017 |
| 66655 | 980379 | 980960 | |
| 66656 | 982566 | 983283 | 065268 |
| 66657 | 983541 | 985323 | 065268 |
| 66658 | 1019475 | 1021960 | 207357, 182087 |
| 66659 | 1025879 | 1027529 | 064666 |
| 66660 | 1093451 | 1095780 | 099817 |
| 66661 | 1097569 | 1100474 | |
| 66662 | 1101115 | 1105693 | 167468 |
| 66663 | 1113054 | 1114327 | 064932 |
| 66664 | 1154206 | 1155893 | 064932 |
| 66665 | 1168074 | 1169999 | 064932 |
| 66666 | 1172677 | 1174885 | 064932 |
| 66667 | 1176759 | 1178365 | |
| 66668 | 1180003 | 1182254 | |
| 66669 | 1182816 | 1184902 | |
| 66670 | 1205092 | 1207287 | 118046 |
| 66671 | 1207341 | 1208125 | 118046 |
| 66672 | 1240062 | 1240635 | |
| 66673 | 1241512 | 1242869 | 099624 |
| 66674 | 1244082 | 1246828 | 099624 |
| 66675 | 1247177 | 1255308 | 167470 |
| 66676 | 1258938 | 1263202 | 099622, 167470 |
| 66677 | 1264620 | 1267544 | 099622 |
| 66678 | 1269015 | 1270994 | 267493, 099622 |
| 66679 | 1274623 | 1276788 | 228300, 099622 |
| 66680 | 1282963 | 1285004 | |
| 66681 | 1304153 | 1305040 | 160953 |
| 66682 | 1313422 | 1314023 | 160953 |
| 66683 | 1324594 | 1325783 | 160953 |
| 66684 | 1353977 | 1356824 | 160953 |
| 66685 | 1373095 | 1374681 | 160953 |
| 66686 | 1382967 | 1384925 | 115286 |
| 66687 | 1395280 | 1396206 | 115286, 248015 |
| 66688 | 1399859 | 1401803 | 130005 |
| 66689 | 1406573 | 1409435 | 071626 |
| 66690 | 1415304 | 1416387 | 071626 |
| 66691 | 1437584 | 1439857 | 115268 |
| 66692 | 1469010 | 1469543 | 115266, 119559 |
| 66693 | 1478313 | 1479997 | 119559 |
| 66694 | 1490385 | 1493665 | 115255, 115257 |
| 66695 | 1500993 | 1501689 | |
| 66696 | 1512527 | 1514309 | 185761 |
| 66697 | 1587784 | 1588706 | 267059, 071655 |
| 66698 | 1591094 | 1593376 | 071655, 267059 |
| 66699 | 1604429 | 1606849 | 127540, 267059 |
| 66700 | 1652324 | 1653844 | 071564 |
| 66701 | 1747332 | 1748315 | 267073 |
| 66702 | 1811449 | 1812479 | 130270 |
| 66703 | 1812553 | 1813304 | |
| 66704 | 1826032 | 1827131 | 079313 |
| 66705 | 1832998 | 1834669 | 079313 |
| 66706 | 1837581 | 1838200 | 079313 |
| 66707 | 1847208 | 1849134 | 079313 |
| 66708 | 1857078 | 1858250 | 129911 |
| 66709 | 1861924 | 1865236 | 267141, 129911 |
| 66710 | 1870642 | 1871953 | |
| 66711 | 1875552 | 1877037 | 129968, 267232, 261526 |
| 66712 | 1884295 | 1885661 | 129968 |
| 66713 | 1904903 | 1906280 | 213638, 227500 |
| 66714 | 1928897 | 1930450 | |
| 66715 | 1939802 | 1943150 | 133275 |
| 66716 | 2014727 | 2016021 | 133243 |
| 66717 | 2031802 | 2033180 | 133243 |
| 66718 | 2041253 | 2043176 | 099875 |
| 66719 | 2047245 | 2052665 | 099875 |
| 66720 | 2057855 | 2062694 | |
| 66721 | 2087021 | 2087532 | 172081 |
| 66722 | 2095194 | 2096775 | 099840, 172081 |
| 66723 | 2149790 | 2151894 | 065000 |
| 66724 | 2163101 | 2165795 | 104885, 065000 |
| 66725 | 2167825 | 2170333 | 104885 |
| 66726 | 2234950 | 2237626 | 104897, 104886 |
| 66727 | 2268381 | 2271826 | 104904, 167476 |
| 66728 | 2273563 | 2274678 | 188305 |
| 66729 | 2327794 | 2329602 | 005206, 130332 |
| 66730 | 2394312 | 2395434 | 178297 |
| 66731 | 2395795 | 2397005 | 178297 |
| 66732 | 2426377 | 2428229 | 176619, 099800 |
| 66733 | 2455640 | 2457334 | 176619 |
| 66734 | 2458149 | 2458673 | |
| 66735 | 2462525 | 2463582 | |
| 66736 | 2474316 | 2480985 | 099860 |
| 66737 | 2488068 | 2489863 | |
| 66738 | 2490607 | 2492168 | |
| 66739 | 2494030 | 2496367 | |
| 66740 | 2506672 | 2507955 | |
| 66741 | 2524744 | 2525896 | 176533 |
| 66742 | 2528360 | 2530547 | 176533 |
| 66743 | 2531914 | 2536927 | 176533 |
| 66744 | 2539378 | 2548199 | 176533 |
| 66745 | 2557272 | 2558186 | 176533 |
| 66746 | 2575841 | 2580469 | 176533 |
| 66747 | 2587158 | 2589254 | 176533 |
| 66748 | 2591553 | 2592612 | 176533 |
| 66749 | 2598582 | 2599881 | 176533 |
| 66750 | 2602336 | 2603081 | 176533 |
| 66751 | 2610368 | 2611789 | 267412, 176533 |
| 66752 | 2624451 | 2632215 | 267214, 176533 |
| 66753 | 2632429 | 2637428 | 267214, 176533 |
| 66754 | 2739284 | 2740472 | 141873, 267001 |
| 66755 | 2775464 | 2776747 | 104969 |
| 66756 | 2781971 | 2783790 | 104969 |
| 66757 | 2784954 | 2786462 | 172009 |
| 66758 | 2790131 | 2791630 | 172009 |
| 66759 | 2818929 | 2820794 | 172006 |
| 66760 | 2840634 | 2842755 | 186300 |
| 66761 | 2900294 | 2901668 | 171970 |
| 66762 | 2944116 | 2945920 | 175691 |
| 66763 | 2949433 | 2951388 | |
| 66764 | 2977018 | 2978302 | 104953 |
| 66765 | 3034156 | 3037053 | 065717 |
| 66766 | 3061207 | 3063218 | 104964 |
| 66767 | 3093750 | 3095835 | 088256 |
| 66768 | 3184654 | 3187009 | 125912 |
| 66769 | 3334607 | 3338130 | |
| 66770 | 3338301 | 3339320 | |
| 66771 | 3343385 | 3344522 | |
| 66772 | 3345908 | 3347600 | |
| 66773 | 3349855 | 3350994 | |
| 66774 | 3358450 | 3362094 | 141905 |
| 66775 | 3365139 | 3371455 | 141905 |
| 66776 | 3373474 | 3376602 | 141905 |
| 66777 | 3384195 | 3385459 | 141905 |
| 66778 | 3386457 | 3389813 | 141905 |
| 66779 | 3397135 | 3399398 | 141905 |
| 66780 | 3402926 | 3405831 | 141905 |
| 66781 | 3407342 | 3414806 | 141905 |
| 66782 | 3415620 | 3416555 | 141905 |
| 66783 | 3428189 | 3429210 | 141905 |
| 66784 | 3429465 | 3431105 | 141905 |
| 66785 | 3431531 | 3432817 | 141905 |
| 66786 | 3450344 | 3453166 | 141905 |
| 66787 | 3458969 | 3460691 | 141905 |
| 66788 | 3467806 | 3468310 | 141905 |
| 66789 | 3499894 | 3501214 | 129932 |
| 66790 | 3505283 | 3509780 | 105325 |
| 66791 | 3516754 | 3517292 | 105325 |
| 66792 | 3544905 | 3545673 | 161091, 267436 |
| 66793 | 3552832 | 3553399 | 161091, 267436 |
| 66794 | 3556362 | 3558172 | 267231, 267436, 161091 |
| 66795 | 3572501 | 3573751 | 064961, 161091 |
| 66796 | 3580125 | 3580630 | |
| 66797 | 3625726 | 3627530 | 105298 |
| 66798 | 3644436 | 3645151 | 186111 |
| 66799 | 3657868 | 3658428 | 186111 |
| 66800 | 3699550 | 3700844 | 186111 |
| 66801 | 3708138 | 3708980 | 105289 |
| 66802 | 3710230 | 3710968 | 105289 |
| 66803 | 3760679 | 3763463 | 183617, 011132 |
| 66804 | 3894894 | 3895401 | 167654 |
| 66805 | 3967943 | 3971611 | 167657 |
| 66806 | 3983001 | 3986925 | 167658 |
| 66807 | 4006710 | 4008646 | 105229 |
| 66808 | 4051003 | 4053409 | 178951 |
| 66809 | 4057706 | 4062193 | 268670, 178951 |
| 66810 | 4062892 | 4068265 | 178951 |
| 66811 | 4077116 | 4078657 | |
| 66812 | 4122570 | 4124609 | 126934 |
| 66813 | 4130784 | 4131594 | |
| 66814 | 4134244 | 4134835 | |
| 66815 | 4136668 | 4137162 | |
| 66816 | 4144441 | 4146386 | |
| 66817 | 4147100 | 4147743 | |
| 66818 | 4149444 | 4150128 | |
| 66819 | 4152555 | 4153960 | 060566 |
| 66820 | 4181729 | 4183106 | 077463 |
| 66821 | 4186355 | 4187073 | 089847 |
| 66822 | 4246366 | 4247666 | 105248 |
| 66823 | 4266976 | 4269479 | 105248 |
| 66824 | 4313862 | 4314515 | 105255 |
| 66825 | 4337751 | 4339393 | 178078 |
| 66826 | 4342056 | 4344104 | 008382, 178078, 269425 |
| 66827 | 4345688 | 4346251 | 008382 |
| 66828 | 4373169 | 4375796 | 141985 |
| 66829 | 4376192 | 4380478 | 141985 |
| 66830 | 4387954 | 4392500 | 141985 |
| 66831 | 4399268 | 4400915 | 141985 |
| 66832 | 4402261 | 4403032 | 167670 |
| 66833 | 4448602 | 4449778 | 167671 |
| 66834 | 4456761 | 4458324 | 267011, 167671 |
| 66835 | 4471692 | 4473530 | 167674 |
| 66836 | 4516574 | 4519758 | 167676 |
| 66837 | 4521184 | 4522061 | |
| 66838 | 4537717 | 4538673 | 267385, 171236 |
| 66839 | 4539303 | 4541022 | 267385, 171236 |
| 66840 | 4547168 | 4548005 | 167680 |
| 66841 | 4596139 | 4596906 | |
| 66842 | 4633085 | 4634040 | |
| 66843 | 4639458 | 4640818 | 185361 |
| 66844 | 4654535 | 4655445 | 268565, 185361, 074842 |
| 66845 | 4669448 | 4670717 | 074842 |
| 66846 | 4693271 | 4697035 | 142002 |
| 66847 | 4704190 | 4711597 | 142002 |
| 66848 | 4712524 | 4716576 | 142002 |
| 66849 | 4721996 | 4724971 | 142002 |
| 66850 | 4790761 | 4792331 | 269604, 141965, 268536 |
| 66851 | 4811575 | 4813978 | |
| 66852 | 4815630 | 4816326 | 127666 |
| 66853 | 4828932 | 4829832 | 127666 |
| 66854 | 4830283 | 4832425 | 127666 |
| 66855 | 4842442 | 4843144 | 105355 |
| 66856 | 4866312 | 4868201 | 105355 |
| 66857 | 4870011 | 4870606 | |
| 66858 | 4904485 | 4905073 | 034063 |
| 66859 | 4968588 | 4970194 | 127663 |
| 66860 | 4972146 | 4973231 | 127663 |
| 66861 | 5001580 | 5002562 | 127663 |
| 66862 | 5054368 | 5055674 | 127663 |
| 66863 | 5104705 | 5105520 | 127663 |
| 66864 | 5484231 | 5485058 | |
| 66865 | 5485096 | 5485702 | |
| 66866 | 5621807 | 5624527 | 160633, 130254 |
| 66867 | 5679421 | 5681179 | 167733, 174917, 130255 |
| 66868 | 5689897 | 5691417 | 130255 |
| 66869 | 5717224 | 5721090 | 174898, 196365 |
| 66870 | 5773588 | 5774178 | 174898 |
| 66871 | 5790460 | 5791560 | 141994 |
| 66872 | 5800396 | 5801411 | |
| 66873 | 5805477 | 5806362 | |
| 66874 | 5826599 | 5827155 | 171119 |
| 66875 | 5827915 | 5829646 | 171119 |
| 66876 | 5837799 | 5838681 | 156413 |
| 66877 | 5870826 | 5871529 | 267740, 130383 |
| 66878 | 5872232 | 5872794 | 267740, 130383 |
| 66879 | 5880129 | 5881265 | 267740, 130383 |
| 66880 | 5902829 | 5905231 | 267314, 187650, 174886, |
| | | | 267740, 130383 |
| 66881 | 5913718 | 5914508 | 267571, 267314, 105519 |
| 66882 | 5953187 | 5955163 | 031823 |
| 66883 | 5961721 | 5963307 | 031823 |
| 66884 | 5967949 | 5968405 | 031823 |
| 66885 | 5977057 | 5979078 | 266983, 031823 |
| 66886 | 5987184 | 5987893 | 266983 |
| 66887 | 6056842 | 6057651 | 087903 |
| 66888 | 6110325 | 6111060 | 087903, 267299 |
| 66889 | 6199117 | 6200308 | 087903 |
| 66890 | 6201831 | 6202341 | |
| 66891 | 6233014 | 6233824 | 130382 |
| 66892 | 6259016 | 6259768 | 267563, 130382 |
| 66893 | 6267594 | 6275458 | 130382 |
| 66894 | 6278852 | 6280521 | 130382 |
| 66895 | 6361254 | 6362837 | 125656, 269802 |
| 66896 | 6371976 | 6373612 | 125652 |
| 66897 | 6423733 | 6424833 | 088247 |
| 66898 | 6424854 | 6426167 | 181240 |
| 66899 | 6458867 | 6461050 | 125648 |
| 66900 | 6463993 | 6465175 | 125648, 130545 |
| 66901 | 6516379 | 6517602 | |
| 66902 | 6669754 | 6670566 | 125735 |
| 66903 | 6719065 | 6722144 | 125730 |
| 66904 | 6722366 | 6723702 | 125730 |
| 66905 | 6725300 | 6726007 | 125730 |
| 66906 | 6729211 | 6730674 | 125734, 125730 |
| 66907 | 6735954 | 6740505 | 125733, 125734 |
| 66908 | 6752086 | 6753885 | 125731 |
| 66909 | 6800975 | 6801964 | 141968 |
| 66910 | 6862627 | 6863958 | |
| 66911 | 7069126 | 7070450 | 130544 |
| 66912 | 7134610 | 7135758 | 171105 |
| 66913 | 7145253 | 7146216 | 171105 |
| 66914 | 7196868 | 7197641 | 171105 |
| 66915 | 7197755 | 7200895 | 171105 |
| 66916 | 7202792 | 7203771 | 171105 |
| 66917 | 7224181 | 7224739 | 171105 |
| 66918 | 7256063 | 7259131 | 171105 |
| 66919 | 7287213 | 7288784 | 171105 |
| 66920 | 7292816 | 7295288 | 171105 |
| 66921 | 7296035 | 7296561 | |
| 66922 | 7459014 | 7460753 | 104880, 267852, 268861 |
| 66923 | 7463263 | 7464538 | 268861, 104880 |
| 66924 | 7553528 | 7554907 | 104883 |
| 66925 | 7559022 | 7560067 | 198723, 104883 |
| 66926 | 7580380 | 7581845 | 198816, 267952 |
| 66927 | 7586912 | 7588317 | 090674 |
| 66928 | 7598313 | 7601501 | 032444, 268614, 090674 |
| 66929 | 7660543 | 7662257 | 076826 |
| 66930 | 7683123 | 7684707 | 076924, 076826 |
| 66931 | 7693430 | 7695486 | 229833, 268400, 076924 |
| 66932 | 7701757 | 7702917 | 076944, 268204, 268400 |
| 66933 | 7716862 | 7717497 | |
| 66934 | 7745380 | 7747067 | 181029, 269711 |
| 66935 | 7864336 | 7864965 | |
| 66936 | 7894418 | 7895841 | 142459 |
| 66937 | 7917766 | 7919046 | 142459 |
| 66938 | 7953162 | 7954176 | 171017 |
| 66939 | 7967547 | 7970039 | 076984, 214248 |
| 66940 | 7981349 | 7982049 | 260001 |
| 66941 | 7983383 | 7985946 | 104976, 260500, 260001 |
| 66942 | 8007700 | 8009559 | 104980 |
| 66943 | 8043142 | 8044473 | 066044 |
| 66944 | 8051630 | 8052526 | 066044 |
| 66945 | 8069617 | 8071415 | 066044 |
| 66946 | 8086992 | 8087638 | |
| 66947 | 8150457 | 8151418 | 142449 |
| 66948 | 8219404 | 8220069 | |
| 66949 | 8273545 | 8275334 | 090661 |
| 66950 | 8289303 | 8289751 | 090661, 271717 |
| 66951 | 8304739 | 8306218 | 090661 |
| 66952 | 8310792 | 8311699 | 090661 |
| 66953 | 8315626 | 8316600 | 090661 |
| 66954 | 8333506 | 8334657 | |
| 66955 | 8370710 | 8373816 | 267855, 167774, 167775 |
| 66956 | 8385048 | 8387565 | 233927, 186994, 267855, |
| | | | 167774 |
| 66957 | 8418865 | 8419248 | |
| 66958 | 8419754 | 8421398 | |
| 66959 | 8424947 | 8425882 | |
| 66960 | 8426351 | 8429965 | 167772 |
| 66961 | 8431492 | 8432856 | 167772 |
| 66962 | 8438545 | 8439808 | 269386, 167772 |
| 66963 | 8452479 | 8455939 | 185236, 265390, 269386 |
| 66964 | 8459184 | 8460040 | 185236 |
| 66965 | 8476282 | 8477311 | |
| 66966 | 8477704 | 8478856 | 099785 |
| 66967 | 8486320 | 8487618 | 099785 |
| 66968 | 8487919 | 8489281 | 268931, 099785 |
| 66969 | 8509048 | 8512408 | 099783 |
| 66970 | 8523666 | 8525846 | 099783 |
| 66971 | 8570615 | 8571467 | |
| 66972 | 8577809 | 8579424 | 133250 |
| 66973 | 8590969 | 8592037 | 142347 |
| 66974 | 8675112 | 8676118 | 142303 |
| 66975 | 8739964 | 8741369 | 268480 |
| 66976 | 8747329 | 8748376 | 268480 |
| 66977 | 8749153 | 8750610 | |
| 66978 | 8942307 | 8943778 | 269400, 170948, 167785 |
| 66979 | 9250369 | 9251867 | 130803 |
| 66980 | 9419854 | 9421162 | 196110 |
| 66981 | 9434524 | 9435372 | 188629, 270011, 188321 |
| 66982 | 9545631 | 9546552 | 174652 |
| 66983 | 9648705 | 9649938 | 130818 |
| 66984 | 9694165 | 9695742 | 197961 |
| 66985 | 9731160 | 9733133 | 267106, 171469 |
| 66986 | 9784222 | 9786501 | 171466 |
| 66987 | 9878392 | 9880379 | 196605 |
| 66988 | 9895613 | 9897027 | 196605 |
| 66989 | 9903141 | 9903839 | 196605 |
| 66990 | 9928680 | 9930570 | 127452 |
| 66991 | 9937656 | 9939463 | 198258, 127452 |
| 66992 | 9945852 | 9946819 | 127445 |
| 66993 | 9963898 | 9964926 | 105088 |
| 66994 | 10024245 | 10025787 | 105088 |
| 66995 | 10046556 | 10047566 | 105088 |
| 66996 | 10112444 | 10113272 | 080573 |
| 66997 | 10137968 | 10138859 | |
| 66998 | 10147003 | 10148797 | 167798 |
| 66999 | 10179166 | 10180394 | 167798 |
| 67000 | 10181378 | 10182215 | 167798 |
| 67001 | 10196299 | 10197915 | 130813 |
| 67002 | 10200285 | 10201302 | 267387, 130813 |
| 67003 | 10201521 | 10202096 | 130813 |
| 67004 | 10216719 | 10218099 | 243207, 130810 |
| 67005 | 10223066 | 10224368 | 244165, 243207, 130810 |
| 67006 | 10224521 | 10226210 | 130811, 244165, 243207, |
| | | | 130810 |
| 67007 | 10229302 | 10230918 | 130811 |
| 67008 | 10330389 | 10330864 | 130816 |
| 67009 | 10340877 | 10342511 | 264266, 267534, 130816 |
| 67010 | 10346582 | 10347370 | |
| 67011 | 10362381 | 10363896 | 105364, 266978 |
| 67012 | 10371558 | 10372248 | 266978 |
| 67013 | 10375906 | 10376554 | 266978 |
| 67014 | 10380037 | 10383332 | 090339, 266978 |
| 67015 | 10425652 | 10427101 | 161847, 267303, 167807, |
| | | | 267673 |
| 67016 | 10443063 | 10444449 | 161847 |
| 67017 | 10444535 | 10446796 | 076662 |
| 67018 | 10456835 | 10457719 | |
| 67019 | 10463376 | 10464376 | 105397 |
| 67020 | 10490328 | 10492188 | 105397 |
| 67021 | 10513160 | 10515150 | 221566, 105401 |
| 67022 | 10526644 | 10527778 | 065989, 105401 |
| 67023 | 10609101 | 10609576 | 079999 |
| 67024 | 10612140 | 10614695 | 079999 |
| 67025 | 10654191 | 10656061 | 130734 |
| 67026 | 10675856 | 10677126 | 129347 |
| 67027 | 10763488 | 10766167 | 129351, 267100 |
| 67028 | 10788443 | 10788911 | 129351 |
| 67029 | 10811827 | 10812991 | 213339 |
| 67030 | 10827928 | 10829717 | 207972, 079805 |
| 67031 | 10855553 | 10856204 | 079805 |
| 67032 | 10859321 | 10860569 | 079805 |
| 67033 | 10874624 | 10875545 | 079805 |
| 67034 | 10880123 | 10881228 | 079805 |
| 67035 | 10945946 | 10947903 | 214212, 099203 |
| 67036 | 10981191 | 10983648 | 142453 |
| 67037 | 11009663 | 11010382 | 142453 |
| 67038 | 11038480 | 11040528 | 142444, 130733 |
| 67039 | 11049802 | 11051027 | |
| 67040 | 11057433 | 11058445 | |
| 67041 | 11070599 | 11072804 | 127616 |
| 67042 | 11108632 | 11109447 | 127616 |
| 67043 | 11134852 | 11136098 | 127616 |
| 67044 | 11146330 | 11147057 | 127616 |
| 67045 | 11194488 | 11195308 | |
| 67046 | 11195484 | 11196785 | |
| 67047 | 11198727 | 11204766 | 130164 |
| 67048 | 11204940 | 11207790 | 130164 |
| 67049 | 11224714 | 11225171 | 130164 |
| 67050 | 11248195 | 11250530 | 161888 |
| 67051 | 11252535 | 11254617 | 161888 |
| 67052 | 11262766 | 11264026 | 161888 |
| 67053 | 11266046 | 11267109 | 161888 |
| 67054 | 11268248 | 11268901 | |
| 67055 | 11270325 | 11270868 | |
| 67056 | 11294982 | 11295630 | 197256 |
| 67057 | 11300386 | 11300999 | 197256 |
| 67058 | 11307179 | 11308840 | 197256 |
| 67059 | 11346015 | 11348221 | 130173, 130158 |
| 67060 | 11349422 | 11351124 | 130158, 130173 |
| 67061 | 11372459 | 11373833 | 130158 |
| 67062 | 11375703 | 11377115 | |
| 67063 | 11404148 | 11404977 | |
| 67064 | 11434232 | 11435283 | 267174, 267576, 105514, |
| | | | 130167 |
| 67065 | 11454972 | 11455526 | 183401, 105518, 105514 |
| 67066 | 11456740 | 11457665 | 105518, 105514, 183401 |
| 67067 | 11465716 | 11466727 | 105520 |
| 67068 | 11484856 | 11486925 | 173928, 267277 |
| 67069 | 11489161 | 11489740 | 187266 |
| 67070 | 11529202 | 11530122 | 205517 |
| 67071 | 11545595 | 11547333 | 130175, 198003 |
| 67072 | 11593356 | 11593872 | 267477 |
| 67073 | 11596129 | 11596757 | 267477, 161914 |
| 67074 | 11601504 | 11602110 | 267477, 161914 |
| 67075 | 11615895 | 11616956 | 130159, 267477, 161914 |
| 67076 | 11639116 | 11640366 | 130159 |
| 67077 | 11642827 | 11643260 | |
| 67078 | 11646149 | 11647290 | |
| 67079 | 11669391 | 11670552 | 198551, 130165 |
| 67080 | 11707929 | 11709246 | 198551 |
| 67081 | 11734110 | 11735930 | |
| 67082 | 11750113 | 11751418 | 197332 |
| 67083 | 11848511 | 11850631 | 197933 |
| 67084 | 11877399 | 11879457 | 197044 |
| 67085 | 11908187 | 11908745 | 177599, 267646 |
| 67086 | 11924593 | 11925893 | 171295 |
| 67087 | 11998322 | 11999467 | 198429 |
| 67088 | 12035602 | 12036726 | 196757, 197054, 267179 |
| 67089 | 12098168 | 12099546 | 219665 |
| 67090 | 12145700 | 12147046 | 197647, 257355, 219665 |
| 67091 | 12175093 | 12176398 | 223547 |
| 67092 | 12250394 | 12251554 | 257591, 132010, 213297 |
| 67093 | 12273586 | 12274762 | 196646 |
| 67094 | 12404415 | 12406568 | 197857 |
| 67095 | 12425306 | 12425871 | |
| 67096 | 12440830 | 12444873 | 188868 |
| 67097 | 12511494 | 12512536 | 268744, 196466, 268870 |
| 67098 | 12551120 | 12552299 | 180855, 268870, 269755 |
| 67099 | 12595379 | 12596209 | 269755, 196826, 242852 |
| 67100 | 12605868 | 12607052 | 196826, 242852 |
| 67101 | 12661223 | 12663542 | 230310, 196826, 249709, |
| | | | 269693 |
| 67102 | 12707973 | 12708629 | 188033 |
| 67103 | 12721128 | 12722554 | 173875, 188033 |
| 67104 | 12776918 | 12777885 | 123154, 104774, 269590 |
| 67105 | 12779006 | 12781517 | 269590, 105583, 123154 |
| 67106 | 12791998 | 12793045 | 095059 |
| 67107 | 12806459 | 12808138 | 132004 |
| 67108 | 12831939 | 12835031 | 105576 |
| 67109 | 12843913 | 12845898 | 123144 |
| 67110 | 12847472 | 12849075 | 198356 |
| 67111 | 12868259 | 12868782 | 039987 |
| 67112 | 12884315 | 12884835 | 095066 |
| 67113 | 12885930 | 12886748 | 095066 |
| 67114 | 12888153 | 12890702 | 095066 |
| 67115 | 12892811 | 12896602 | 267212, 095066 |
| 67116 | 12898856 | 12905862 | 171223, 095066 |
| 67117 | 12911810 | 12913255 | 167815, 267062, 095066 |
| 67118 | 12991517 | 12992796 | 267735, 105612 |
| 67119 | 13001537 | 13002747 | 105607 |
| 67120 | 13043514 | 13045248 | 179115, 266975 |
| 67121 | 13048914 | 13050718 | 179218 |
| 67122 | 13056139 | 13058007 | 179262 |
| 67123 | 13067250 | 13068764 | 179271 |
| 67124 | 13105872 | 13106406 | |
| 67125 | 13106572 | 13107764 | 008441 |
| 67126 | 13112382 | 13113445 | 008441 |
| 67127 | 13133081 | 13135577 | 008441 |
| 67128 | 13198538 | 13199507 | 008441 |
| 67129 | 13206993 | 13207665 | 008441 |
| 67130 | 13213072 | 13213736 | 104903 |
| 67131 | 13226070 | 13228165 | 104907 |
| 67132 | 13228693 | 13230598 | 160877 |
| 67133 | 13239216 | 13239818 | 160877 |
| 67134 | 13245326 | 13245689 | 160877 |
| 67135 | 13260166 | 13267514 | 160888, 267598, 104915 |
| 67136 | 13273087 | 13275112 | |
| 67137 | 13277512 | 13279520 | |
| 67138 | 13282904 | 13283522 | |
| 67139 | 13301277 | 13302452 | |
| 67140 | 13306205 | 13307096 | |
| 67141 | 13371354 | 13372252 | 141837 |
| 67142 | 13507337 | 13507746 | 141837 |
| 67143 | 13833705 | 13834501 | |
| 67144 | 13838605 | 13839180 | |
| 67145 | 13841364 | 13843251 | 104957 |
| 67146 | 13858461 | 13859874 | 104957 |
| 67147 | 13875016 | 13876965 | 037757 |
| 67148 | 13885058 | 13886314 | 037757, 104979 |
| 67149 | 13904956 | 13908649 | 132003, 267582 |
| 67150 | 13943852 | 13944986 | |
| 67151 | 13952391 | 13954835 | |
| 67152 | 13957166 | 13959320 | |
| 67153 | 13960659 | 13963037 | |
| 67154 | 13963165 | 13964956 | |
| 67155 | 13972140 | 13973571 | 187556 |
| 67156 | 13990756 | 13992255 | 187556 |
| 67157 | 14016768 | 14018094 | 132024, 132016 |
| 67158 | 14062950 | 14064098 | 132017, 132000 |
| 67159 | 14115383 | 14118500 | 132005 |
| 67160 | 14142308 | 14143406 | 104998, 267670 |
| 67161 | 14171853 | 14173451 | |
| 67162 | 14182306 | 14186373 | 141854 |
| 67163 | 14190923 | 14193560 | 188032 |
| 67164 | 14201005 | 14202648 | 072062, 141858 |
| 67165 | 14228391 | 14230706 | 267783, 105011, 072062 |
| 67166 | 14247153 | 14249084 | 267169, 105011 |
| 67167 | 14528964 | 14530916 | 123136 |
| 67168 | 14543946 | 14544994 | 123143 |
| 67169 | 14606151 | 14607890 | 123159 |
| 67170 | 14613380 | 14613991 | |
| 67171 | 14617780 | 14619505 | |
| 67172 | 14620984 | 14622313 | |
| 67173 | 14623514 | 14624491 | |
| 67174 | 14626654 | 14631134 | 099797, 132002 |
| 67175 | 14639840 | 14641272 | 207707, 132002, 099797 |
| 67176 | 14655894 | 14656725 | 099797 |
| 67177 | 14682140 | 14683253 | 099795 |
| 67178 | 14800179 | 14801372 | 160961, 131355 |
| 67179 | 15217912 | 15218476 | 105137 |
| 67180 | 15235102 | 15236785 | 105135, 268564 |
| 67181 | 15359242 | 15360173 | 141867 |
| 67182 | 15391629 | 15392381 | 141867 |
| 67183 | 15407386 | 15409267 | 141867 |
| 67184 | 15439229 | 15440232 | 141867 |
| 67185 | 15441697 | 15443994 | 141867 |
| 67186 | 15468673 | 15469194 | 105127 |
| 67187 | 15478147 | 15478585 | 105127 |
| 67188 | 15488496 | 15491690 | 011243, 268189, 105127 |
| 67189 | 15522129 | 15522575 | 011243 |
| 67190 | 15528773 | 15530418 | 011243 |
| 67191 | 15542375 | 15544371 | 011451 |
| 67192 | 15560054 | 15561358 | 269782, 011451 |
| 67193 | 15563494 | 15565228 | 105122 |
| 67194 | 15579484 | 15580438 | 161031 |
| 67195 | 15589694 | 15591554 | 161031 |
| 67196 | 15616699 | 15617464 | |
| 67197 | 15625184 | 15626413 | 269765, 171954 |
| 67198 | 15744941 | 15747597 | |
| 67199 | 15750105 | 15756476 | 186529 |
| 67200 | 15783281 | 15784928 | 186204 |
| 67201 | 15791091 | 15792704 | 186204 |
| 67202 | 15946874 | 15948454 | 214049 |
| 67203 | 15979809 | 15980656 | |
| 67204 | 16007736 | 16009233 | 186115 |
| 67205 | 16037027 | 16037946 | 171903 |
| 67206 | 16044626 | 16046850 | 171903 |
| 67207 | 16112626 | 16113393 | |
| 67208 | 16186375 | 16190589 | 167460 |
| 67209 | 16205321 | 16206412 | 167460 |
| 67210 | 16222042 | 16223712 | 167461 |
| 67211 | 16226250 | 16227251 | 167461 |
| 67212 | 16230031 | 16230848 | 167461 |
| 67213 | 16274536 | 16275650 | 141977 |
| 67214 | 16295742 | 16297072 | 105058 |
| 67215 | 16307374 | 16309975 | 072958 |
| 67216 | 16310284 | 16311873 | 072958 |
| 67217 | 16389850 | 16390470 | |
| 67218 | 16403543 | 16405290 | 267275 |
| 67219 | 16435146 | 16436574 | 127528, 267275 |
| 67220 | 16441197 | 16441786 | |
| 67221 | 16473899 | 16474363 | 127527 |
| 67222 | 16572338 | 16574640 | 127527 |
| 67223 | 16574643 | 16575417 | 127527 |
| 67224 | 16580341 | 16583686 | 127527 |
| 67225 | 16603650 | 16604349 | 269058, 141979 |
| 67226 | 16652379 | 16654277 | 269399, 085872, 141979 |
| 67227 | 16682069 | 16684283 | 127526, 141979 |
| 67228 | 16718997 | 16719539 | 105085, 268790, 141979 |
| 67229 | 16722027 | 16723119 | 268087, 105085, 268790, |
| | | | 141979 |
| 67230 | 16737601 | 16740590 | 105085, 141979, 268087, |
| | | | 268790 |
| 67231 | 16770208 | 16773025 | 072954, 268790, 214046 |
| 67232 | 16786942 | 16788061 | 072954 |
| 67233 | 16795875 | 16796772 | 072954 |
| 67234 | 16939994 | 16941147 | 127511 |
| 67235 | 16996973 | 16997781 | |
| 67236 | 17185441 | 17187440 | 099331, 131351 |
| 67237 | 17208195 | 17208960 | 099331, 269066 |
| 67238 | 17218883 | 17219307 | 099331 |
| 67239 | 17244897 | 17245407 | 099331 |
| 67240 | 17325160 | 17326806 | 053501, 099331 |
| 67241 | 17336635 | 17338278 | 099330 |
| 67242 | 17350575 | 17352007 | 160113, 269095 |
| 67243 | 17352991 | 17358435 | 160113, 269095 |
| 67244 | 17377868 | 17379306 | 105393, 269307, 130307 |
| 67245 | 17413263 | 17414667 | 127220, 130312 |
| 67246 | 17415831 | 17417850 | 130312, 127220 |
| 67247 | 17420020 | 17421494 | 130311, 127220 |
| 67248 | 17422427 | 17423878 | 130311 |
| 67249 | 17444791 | 17446230 | 130299, 074855 |
| 67250 | 17447215 | 17448601 | 130299 |
| 67251 | 17451569 | 17452184 | 130299 |
| 67252 | 17515161 | 17518025 | 269640, 141971, 130303 |
| 67253 | 17530013 | 17531596 | 269053, 269481, 141971 |
| 67254 | 17618706 | 17619698 | 269439 |
| 67255 | 17621355 | 17623757 | 130313, 269439 |
| 67256 | 17666104 | 17667547 | 130309 |
| 67257 | 17829884 | 17831220 | 130479 |
| 67258 | 17901044 | 17901780 | |
| 67259 | 17907229 | 17908868 | 179913 |
| 67260 | 17970161 | 17972298 | 105640 |
| 67261 | 18042699 | 18045106 | 007080 |
| 67262 | 18058325 | 18059495 | |
| 67263 | 18111258 | 18113142 | 105643 |
| 67264 | 18117484 | 18120565 | 105643 |
| 67265 | 18123725 | 18125534 | 105643 |
| 67266 | 18130497 | 18133826 | 268032 |
| 67267 | 18140160 | 18141335 | 268032 |
| 67268 | 18155665 | 18156579 | |
| 67269 | 18156851 | 18157823 | |
| 67270 | 18161765 | 18163290 | |
| 67271 | 18214788 | 18216060 | 099308 |
| 67272 | 18219405 | 18221927 | 099308 |
| 67273 | 18228371 | 18229098 | 099308 |
| 67274 | 18246212 | 18247753 | 099308 |
| 67275 | 18262189 | 18265595 | 268173, 105647, 269145, |
| | | | 099308 |
| 67276 | 18284163 | 18285256 | 268173, 216490 |
| 67277 | 18302454 | 18304882 | 254858 |
| 67278 | 18314778 | 18316332 | 268650, 105649 |
| 67279 | 18334733 | 18335638 | 105650 |
| 67280 | 18384685 | 18386958 | 130518 |
| 67281 | 18389542 | 18393956 | 130522, 267959 |
| 67282 | 18401959 | 18404710 | |
| 67283 | 18411336 | 18412427 | |
| 67284 | 18414941 | 18416409 | |
| 67285 | 18432949 | 18434935 | 130520 |
| 67286 | 18439090 | 18440057 | 67995 |
| 67287 | 18451012 | 18452508 | 130517 |
| 67288 | 18468154 | 18468983 | 130517 |
| 67289 | 18473779 | 18477521 | 130517 |
| 67290 | 18479255 | 18479872 | 130517 |
| 67291 | 18483592 | 18486218 | 130513 |
| 67292 | 18487658 | 18492040 | 130513 |
| 67293 | 18493380 | 18494526 | 130513 |
| 67294 | 18495850 | 18498727 | 264175, 130513 |
| 67295 | 18499084 | 18500294 | 130513 |
| 67296 | 18506720 | 18507918 | 175489 |
| 67297 | 18509765 | 18510314 | |
| 67298 | 18529959 | 18531038 | 130511 |
| 67299 | 18557711 | 18558341 | 105656 |
| 67300 | 18575242 | 18575900 | 105656 |
| 67301 | 18579927 | 18581476 | 105656 |
| 67302 | 18589003 | 18590506 | 105656 |
| 67303 | 18606914 | 18607610 | 105656 |
| 67304 | 18614693 | 18616126 | 105656 |
| 67305 | 18625340 | 18626516 | 105656 |
| 67306 | 18630470 | 18633864 | 105656 |
| 67307 | 18648795 | 18649303 | 105701 |
| 67308 | 18652916 | 18655110 | 105701 |
| 67309 | 18682054 | 18684041 | 221983, 006016 |
| 67310 | 18698892 | 18701077 | 006015, 006016 |
| 67311 | 18747527 | 18748846 | 167487 |
| 67312 | 18793648 | 18794888 | 105662 |
| 67313 | 18942346 | 18944377 | 005007 |
| 67314 | 19009320 | 19009855 | |
| 67315 | 19029262 | 19031738 | 105671, 105669, 268193 |
| 67316 | 19051334 | 19052315 | 051128, 269019 |
| 67317 | 19143522 | 19145440 | 105676, 064607 |
| 67318 | 19151185 | 19151889 | 105676 |
| 67319 | 19162090 | 19164007 | 105676 |
| 67320 | 19173135 | 19175545 | 181035 |
| 67321 | 19206425 | 19210694 | 181035 |
| 67322 | 19221948 | 19223266 | 181035 |
| 67323 | 19248280 | 19249867 | 064545 |
| 67324 | 19302483 | 19304320 | 064490, 064489, 254901, |
| | | | 213999 |
| 67325 | 19313280 | 19314514 | 184162 |
| 67326 | 19339200 | 19339751 | 130287 |
| 67327 | 19383455 | 19384389 | 267629, 213996 |
| 67328 | 19385306 | 19386268 | |
| 67329 | 19430594 | 19432802 | 129933, 105705 |
| 67330 | 19495417 | 19497834 | 167491 |
| 67331 | 19513837 | 19515212 | 167491 |
| 67332 | 19515422 | 19518199 | 167491 |
| 67333 | 19534172 | 19534761 | 167491 |
| 67334 | 19545173 | 19545988 | 207821, 167491 |
| 67335 | 19598340 | 19601590 | 167491 |
| 67336 | 19626280 | 19627850 | 250067, 258674, 186010, |
| | | | 178093 |
| 67337 | 19773656 | 19775330 | 105726 |
| 67338 | 19779237 | 19780528 | 181896 |
| 67339 | 19886494 | 19888295 | 267419, 266904, 267581 |
| 67340 | 19976412 | 19978066 | 256771 |
| 67341 | 20149262 | 20150585 | 197124 |
| 67342 | 20162491 | 20163827 | |
| 67343 | 21511156 | 21512484 | 182141 |
| 67344 | 21579546 | 21580332 | 196268 |
| 67345 | 21675615 | 21676567 | 268658 |
| 67346 | 21688163 | 21689900 | 197013 |
| 67347 | 21769009 | 21770082 | 268555 |
| 67348 | 21776795 | 21777677 | 268240 |
| 67349 | 21949416 | 21950500 | 197020 |
| 67350 | 22300915 | 22301301 | |
| 67351 | 23444122 | 23445124 | 269107, 213971 |
| 67352 | 23445606 | 23447445 | 269107, 213971 |
| 67353 | 23455892 | 23457350 | 269107, 213971 |
| 67354 | 23577154 | 23578696 | 167232 |
| 67355 | 23869124 | 23870273 | 197372 |
| 67356 | 24065072 | 24066024 | |
| 67357 | 24070334 | 24070911 | |
| 67358 | 24126062 | 24126643 | 269289, 213967 |
| 67359 | 24169387 | 24169995 | |
| 67360 | 24174927 | 24175726 | |
| 67361 | 24215923 | 24217906 | 213096, 268362 |
| 67362 | 24236443 | 24237754 | 213096, 268362 |
| 67363 | 27738278 | 27738762 | |
| 67364 | 28283696 | 28285922 | 267575, 272635, 261824 |
| 67365 | 28971698 | 28973141 | 267243 |
| 67366 | 29010900 | 29011564 | 267243, 266893 |
| 67367 | 29060245 | 29061274 | 267243, 266893 |
| 67368 | 29457755 | 29458268 | 267339 |
| 67369 | 29702656 | 29704706 | 267498, 169021 |
| 67370 | 30096307 | 30098026 | 105171 |
| 67371 | 30105719 | 30106158 | 105171 |
| 67372 | 30136932 | 30137342 | |
| 67373 | 30155559 | 30158256 | 166289 |
| 67374 | 30159025 | 30160510 | 166289 |
| 67375 | 30161926 | 30162932 | 166289 |
| 67376 | 30167319 | 30168193 | |
| 67377 | 30169366 | 30170136 | |
| 67378 | 30195690 | 30196228 | 131943 |
| 67379 | 30196493 | 30197981 | 131943 |
| 67380 | 30198041 | 30200684 | 131943 |
| 67381 | 30205427 | 30207939 | 131943 |
| 67382 | 30295908 | 30297305 | |
| 67383 | 30301901 | 30303600 | 105173 |
| 67384 | 30432808 | 30434229 | 105176 |
| 67385 | 30527246 | 30528094 | |
| 67386 | 31152721 | 31154034 | 198597 |
| 67387 | 31154785 | 31156040 | 198597 |
| 67388 | 31209513 | 31211586 | |
| 67389 | 31841223 | 31842482 | 267662 |
| 67390 | 32715276 | 32716107 | |
| 67391 | 32836117 | 32837856 | 168813 |
| 67392 | 32854077 | 32854600 | 168813 |
| 67393 | 32863222 | 32865284 | 168813 |
| 67394 | 32895474 | 32897719 | 178904, 269093, 267213 |
| 67395 | 33071279 | 33073298 | 105185 |
| 67396 | 33165185 | 33166271 | 105186 |
| 67397 | 33182248 | 33183997 | 213965, 267475 |
| 67398 | 33187636 | 33188151 | 213965 |
| 67399 | 33192889 | 33193441 | 213965 |
| 67400 | 33360040 | 33361493 | 021488 |
| 67401 | 33462686 | 33463504 | 131944, 121289 |
| 67402 | 33529628 | 33533821 | 131941 |
| 67403 | 33543036 | 33543858 | 131941 |
| 67404 | 33554580 | 33556603 | 131941 |
| 67405 | 33568941 | 33569341 | |
| 67406 | 33571300 | 33572903 | 076650 |
| 67407 | 33605044 | 33605711 | 076650 |
| 67408 | 33666922 | 33669062 | 267054, 264355, 130881 |
| 67409 | 33685297 | 33686759 | 130881 |
| 67410 | 33731622 | 33733310 | |
| 67411 | 33738043 | 33740919 | |
| 67412 | 33764787 | 33765984 | |
| 67413 | 33766328 | 33767704 | |
| 67414 | 33768897 | 33770155 | |
| 67415 | 33776272 | 33777338 | 270212 |
| 67416 | 33781946 | 33785423 | |
| 67417 | 33788215 | 33796775 | 267727, 230259, 267296, |
| | | | 245848, 267130, 178863, |
| | | | 267580 |
| 67418 | 33807097 | 33808327 | |
| 67419 | 33816795 | 33817362 | |
| 67420 | 33839245 | 33841039 | |
| 67421 | 33863591 | 33868975 | 153879 |
| 67422 | 33882889 | 33886500 | 124299 |
| 67423 | 33887953 | 33888802 | 124299 |
| 67424 | 33890622 | 33892484 | 124299 |
| 67425 | 33893011 | 33898331 | 124299 |
| 67426 | 33928514 | 33929734 | 124299 |
| 67427 | 33989470 | 33991209 | 124299 |
| 67428 | 33992776 | 33993401 | 124299 |
| 67429 | 34010262 | 34010684 | 124299 |
| 67430 | 34011101 | 34013000 | 124299 |
| 67431 | 34285750 | 34288488 | 153885 |
| 67432 | 34294734 | 34296081 | 153885 |
| 67433 | 34391726 | 34392183 | |
| 67434 | 34396264 | 34397701 | |
| 67435 | 34624947 | 34626474 | |
| 67436 | 34662217 | 34665036 | 257103 |
| 67437 | 34672222 | 34673167 | 257103 |
| 67438 | 34682483 | 34682949 | 257103 |
| 67439 | 34723127 | 34725053 | |
| 67440 | 34744777 | 34746575 | 166398 |
| 67441 | 34777876 | 34778456 | 166398 |
| 67442 | 34796341 | 34798359 | 166398 |
| 67443 | 34819830 | 34820271 | 166398 |
| 67444 | 34828454 | 34829171 | 166398 |
| 67445 | 34849301 | 34851324 | 105220, 267219 |
| 67446 | 34855706 | 34856933 | 105220 |
| 67447 | 34894885 | 34895942 | 126249, 266953 |
| 67448 | 34918631 | 34920565 | 126261, 267024 |
| 67449 | 35146573 | 35146992 | 205209 |
| 67450 | 35157771 | 35159049 | 205209 |
| 67451 | 35167190 | 35169570 | 089335, 256383 |
| 67452 | 35223733 | 35226210 | 197841 |
| 67453 | 35417607 | 35418636 | 168661, 270876 |
| 67454 | 35454283 | 35455309 | 180884 |
| 67455 | 35466665 | 35467270 | |
| 67456 | 35485580 | 35486709 | 089351 |
| 67457 | 35490197 | 35492340 | 269303, 089351 |
| 67458 | 35496322 | 35498399 | 089351 |
| 67459 | 35521474 | 35522072 | 105711 |
| 67460 | 35530520 | 35534795 | 105707, 10571 1 |
| 67461 | 35539570 | 35540475 | 105707 |
| 67462 | 35571678 | 35572704 | 227392 |
| 67463 | 35581085 | 35582107 | 227392 |
| 67464 | 35626150 | 35627032 | 153902 |
| 67465 | 35627686 | 35628623 | 203403, 153902 |
| 67466 | 35629098 | 35631709 | 266964, 221857, 203403, |
| | | | 153902 |
| 67467 | 35645229 | 35646252 | 089327 |
| 67468 | 35661614 | 35662324 | |
| 67469 | 35686508 | 35688334 | |
| 67470 | 35738452 | 35740966 | 105699 |
| 67471 | 35750379 | 35750942 | 105699 |
| 67472 | 35757790 | 35761023 | 105698, 105699 |
| 67473 | 35809186 | 35810442 | 012124 |
| 67474 | 35911012 | 35911962 | |
| 67475 | 35950002 | 35950871 | |
| 67476 | 36035397 | 36037335 | 105677, 236144, 105679 |
| 67477 | 36103414 | 36103932 | 249115, 267626 |
| 67478 | 36119088 | 36120901 | 126254 |
| 67479 | 36133621 | 36134731 | 105672 |
| 67480 | 36139021 | 36140199 | 126267 |
| 67481 | 36151842 | 36152895 | |
| 67482 | 36206673 | 36210361 | 272333, 105663, 011590 |
| 67483 | 36231134 | 36232283 | 126246, 267120 |
| 67484 | 36235660 | 36237426 | 205155, 188223, 161265, |
| | | | 267120 |
| 67485 | 36238981 | 36240000 | 267796, 267439, 188223 |
| 67486 | 36247886 | 36249716 | 167595, 004776 |
| 67487 | 36251774 | 36252305 | 167595 |
| 67488 | 36302329 | 36304370 | 250799 |
| 67489 | 36390011 | 36391790 | 167604 |
| 67490 | 36422195 | 36423085 | |
| 67491 | 36427019 | 36427943 | 126243 |
| 67492 | 36433087 | 36433600 | 126243 |
| 67493 | 36485311 | 36487565 | 205138 |
| 67494 | 36490055 | 36490775 | |
| 67495 | 36504300 | 36505728 | 105270, 267698, 248101, |
| | | | 239382 |
| 67496 | 36521211 | 36521762 | 105270, 267698 |
| 67497 | 36532717 | 36533149 | 161277, 267698 |
| 67498 | 36544812 | 36545991 | 075702, 161277 |
| 67499 | 36604753 | 36607210 | 105254, 105258 |
| 67500 | 36618171 | 36619988 | |
| 67501 | 36627630 | 36628502 | |
| 67502 | 36629889 | 36632422 | 126247 |
| 67503 | 36704619 | 36706925 | 167635, 196357 |
| 67504 | 36868634 | 36870151 | 266973, 142065 |
| 67505 | 36979381 | 36981492 | 267309, 186017 |
| 67506 | 37018162 | 37020485 | 228629, 254004 |
| 67507 | 37063129 | 37065502 | 233527, 186020 |
| 67508 | 37095997 | 37096801 | 186020, 161298 |
| 67509 | 37157327 | 37158604 | 197808 |
| 67510 | 37177420 | 37179257 | 189042, 225975 |
| 67511 | 37263464 | 37264064 | 267260, 267041 |
| 67512 | 37328275 | 37330124 | 197863 |
| 67513 | 37340389 | 37342278 | 251247, 197863 |
| 67514 | 37568484 | 37570267 | 267345, 197050 |
| 67515 | 37663221 | 37664261 | 196967, 267360 |
| 67516 | 37708347 | 37709939 | 188283, 245680 |
| 67517 | 37808342 | 37809468 | 181666 |
| 67518 | 37825368 | 37826390 | 181666 |
| 67519 | 37861497 | 37862491 | 189164 |
| 67520 | 37957551 | 37958728 | 171827, 196437 |
| 67521 | 37959820 | 37960935 | 196437, 171827 |
| 67522 | 38084916 | 38086450 | 180479, 171817 |
| 67523 | 38145462 | 38147188 | 120784 |
| 67524 | 38269472 | 38270880 | 267422, 189144 |
| 67525 | 38396705 | 38398643 | 105738, 171804 |
| 67526 | 38422540 | 38425343 | 105738 |
| 67527 | 38426438 | 38427328 | 105738 |
| 67528 | 38431727 | 38432763 | 105738 |
| 67529 | 38433861 | 38434467 | 105738 |
| 67530 | 38482445 | 38482973 | 105738 |
| 67531 | 38741081 | 38741910 | 167641, 167642 |
| 67532 | 38790795 | 38792243 | 267748 |
| 67533 | 38805669 | 38807532 | 167645 |
| 67534 | 38826059 | 38827468 | 099338 |
| 67535 | 38864769 | 38866622 | 099341 |
| 67536 | 38893207 | 38894660 | 130244 |
| 67537 | 39034403 | 39035664 | 196218 |
| 67538 | 39108899 | 39110907 | 178982, 104814 |
| 67539 | 39127394 | 39127913 | 178982 |
| 67540 | 39137811 | 39145084 | 130402 |
| 67541 | 39145172 | 39146403 | 130402 |
| 67542 | 39146660 | 39151938 | 130402 |
| 67543 | 39157054 | 39158441 | 130402 |
| 67544 | 39161473 | 39162298 | 130402 |
| 67545 | 39169986 | 39177821 | 267375, 130402 |
| 67546 | 39179377 | 39180313 | 130402, 267375 |
| 67547 | 39191670 | 39192853 | 130402 |
| 67548 | 39198162 | 39198949 | 130402 |
| 67549 | 39202201 | 39203194 | 130402 |
| 67550 | 39203622 | 39205579 | 130402 |
| 67551 | 39226742 | 39227430 | 182472 |
| 67552 | 39249743 | 39250970 | 182472 |
| 67553 | 39251702 | 39255486 | 182472 |
| 67554 | 39303196 | 39304230 | 171747 |
| 67555 | 39320510 | 39323918 | 104823, 268083 |
| 67556 | 39325776 | 39327208 | 104824, 268083 |
| 67557 | 39338506 | 39344000 | 104824 |
| 67558 | 39355220 | 39356060 | |
| 67559 | 39359684 | 39361318 | 269050, 187994 |
| 67560 | 39389534 | 39392037 | 104825, 068903 |
| 67561 | 39395128 | 39396070 | 104825 |
| 67562 | 39420658 | 39422471 | 128626, 269547, 104835 |
| 67563 | 39442812 | 39443863 | 269190 |
| 67564 | 39465375 | 39467631 | 269190 |
| 67565 | 39521603 | 39523214 | 267992, 161243 |
| 67566 | 39602074 | 39603615 | 183760 |
| 67567 | 39615794 | 39617756 | 130669 |
| 67568 | 39684394 | 39685038 | |
| 67569 | 39767990 | 39768766 | |
| 67570 | 39831583 | 39834260 | 269246, 179134, 130755 |
| 67571 | 39867937 | 39869508 | 179134 |
| 67572 | 39880937 | 39882679 | 063322, 006712 |
| 67573 | 39887705 | 39890959 | 063322 |
| 67574 | 39891151 | 39895094 | 063322 |
| 67575 | 39896347 | 39904084 | 128016, 090924, 266559 |
| 67576 | 39925737 | 39927764 | 196235, 105193 |
| 67577 | 39935002 | 39937500 | 196235 |
| 67578 | 39971054 | 39972650 | 105197 |
| 67579 | 40022705 | 40023811 | 269792, 176401 |
| 67580 | 40029732 | 40031184 | 176396 |
| 67581 | 40048608 | 40049512 | |
| 67582 | 40168462 | 40169243 | |
| 67583 | 40323601 | 40325217 | 105202, 105204 |
| 67584 | 40336314 | 40337754 | 105202 |
| 67585 | 40449713 | 40450348 | |
| 67586 | 40475232 | 40478143 | 013275 |
| 67587 | 40502087 | 40503972 | 187187 |
| 67588 | 40561715 | 40562520 | 128000, 269749 |
| 67589 | 40595973 | 40597775 | 269296, 197782, 269749 |
| 67590 | 40695763 | 40698519 | 130758 |
| 67591 | 40731658 | 40733124 | 105219 |
| 67592 | 40762826 | 40763508 | 105221 |
| 67593 | 40786061 | 40787101 | 105221 |
| 67594 | 40789191 | 40792229 | 105221 |
| 67595 | 40853976 | 40855372 | 105223, 160392 |
| 67596 | 40926423 | 40928120 | 197019 |
| 67597 | 40930378 | 40932697 | 197019 |
| 67598 | 40938881 | 40940470 | |
| 67599 | 40948773 | 40950812 | 268366, 167565 |
| 67600 | 40967617 | 40968195 | 090013 |
| 67601 | 40969817 | 40973390 | 160460, 090013 |
| 67602 | 41029035 | 41029711 | 160460 |
| 67603 | 41081910 | 41083667 | 160410, 160460 |
| 67604 | 41109542 | 41110139 | 090006 |
| 67605 | 41115193 | 41116183 | 090006 |
| 67606 | 41168301 | 41170239 | |
| 67607 | 41171676 | 41173516 | 105245 |
| 67608 | 41195896 | 41197613 | 123815, 105245 |
| 67609 | 41220110 | 41225990 | 086544, 123815 |
| 67610 | 41255165 | 41258224 | 077312, 188493 |
| 67611 | 41259923 | 41261414 | 077312 |
| 67612 | 41283522 | 41284767 | 171570, 167578, 268975 |
| 67613 | 41303799 | 41306498 | 269858, 171570 |
| 67614 | 41328984 | 41330560 | 268797, 237118 |
| 67615 | 41337189 | 41338419 | 268797, 269843 |
| 67616 | 41402995 | 41403531 | 268797, 130612 |
| 67617 | 41426329 | 41428449 | |
| 67618 | 41485290 | 41486679 | |
| 67619 | 41488286 | 41488967 | |
| 67620 | 41495191 | 41496355 | |
| 67621 | 41496365 | 41498393 | 197408 |
| 67622 | 41500408 | 41501851 | 197408 |
| 67623 | 41506084 | 41506732 | 197408 |
| 67624 | 41645931 | 41646389 | |
| 67625 | 41681898 | 41682527 | |
| 67626 | 41730813 | 41733315 | 167601 |
| 67627 | 41767228 | 41772161 | 105323, 167601 |
| 67628 | 41814200 | 41814869 | 105329 |
| 67629 | 41815174 | 41817095 | 142039, 105329 |
| 67630 | 41859546 | 41860266 | 105329, 255730, 142046 |
| 67631 | 41869462 | 41870895 | 123810, 255730, 142046 |
| 67632 | 41902596 | 41904919 | 077348, 248098, 255730 |
| 67633 | 41915057 | 41916591 | 248098, 255730 |
| 67634 | 41945246 | 41946129 | 105341 |
| 67635 | 42363399 | 42365724 | 105372 |
| 67636 | 42386604 | 42389303 | 076928 |
| 67637 | 42462681 | 42464077 | 105404 |
| 67638 | 42578964 | 42581223 | 105732 |
| 67639 | 42720685 | 42723181 | 160570 |
| 67640 | 42723893 | 42725155 | 167625, 160570 |
| 67641 | 42745514 | 42749339 | 204957, 105723, 268643 |
| 67642 | 42756361 | 42760396 | 105722, 268643 |
| 67643 | 42772067 | 42776080 | 079432 |
| 67644 | 42783904 | 42785018 | 079432 |
| 67645 | 42787907 | 42788919 | 079432 |
| 67646 | 42800036 | 42800628 | |
| 67647 | 42829129 | 42830691 | 105429, 167619 |
| 67648 | 42882636 | 42883399 | 105429 |
| 67649 | 42891148 | 42891802 | 105427 |
| 67650 | 42913512 | 42914233 | 213904, 079435 |
| 67651 | 42937137 | 42938407 | 213904, 189377 |
| 67652 | 42953972 | 42955092 | 213904 |
| 67653 | 42959604 | 42960360 | 213904 |
| 67654 | 42984807 | 42985440 | 213904 |
| 67655 | 42998316 | 42999183 | 213904 |
| 67656 | 43001400 | 43002949 | 213904 |
| 67657 | 43032009 | 43034037 | 213904, 079385 |
| 67658 | 43704318 | 43706519 | 243137 |
| 67659 | 44002748 | 44003934 | 176531 |
| 67660 | 44007174 | 44009461 | 176531 |
| 67661 | 44029234 | 44031956 | 176472, 105755 |
| 67662 | 44036376 | 44037679 | 176472 |
| 67663 | 44098750 | 44101737 | 124444, 226763, 167378 |
| 67664 | 44122555 | 44124544 | 131116 |
| 67665 | 44257964 | 44260025 | 105771 |
| 67666 | 44331196 | 44332113 | 167637, 159871 |
| 67667 | 44438714 | 44439926 | 124459 |
| 67668 | 44454931 | 44455860 | 159905 |
| 67669 | 44487890 | 44488975 | 204920 |
| 67670 | 44506071 | 44508056 | 159882, 266921 |
| 67671 | 44529270 | 44530333 | 267022, 159885, 267144 |
| 67672 | 44555456 | 44556815 | 178386, 267022 |
| 67673 | 44597979 | 44599033 | 267680, 267163 |
| 67674 | 44616797 | 44618421 | 186019, 256294 |
| 67675 | 44645049 | 44646645 | 263002 |
| 67676 | 44668541 | 44669975 | 167380 |
| 67677 | 44711004 | 44712379 | 131115 |
| 67678 | 44716289 | 44717407 | 131115 |
| 67679 | 44808330 | 44809337 | 159917 |
| 67680 | 45004056 | 45005277 | 167384 |
| 67681 | 45091013 | 45093498 | 230666 |
| 67682 | 45095103 | 45096347 | 230666 |
| 67683 | 45106360 | 45107175 | 230666 |
| 67684 | 45115911 | 45127130 | 216588, 230666 |
| 67685 | 45128017 | 45131310 | 216588 |
| 67686 | 45143174 | 45144473 | 266903 |
| 67687 | 45144525 | 45148423 | 073008, 266903 |
| 67688 | 45151698 | 45152721 | 073008, 266903 |
| 67689 | 45157572 | 45158141 | 073008, 266903 |
| 67690 | 45180548 | 45180981 | 186567, 266903 |
| 67691 | 45192340 | 45193763 | 266903 |
| 67692 | 45194402 | 45196780 | 266903 |
| 67693 | 45197009 | 45199410 | 266903 |
| 67694 | 45221246 | 45222856 | 266903 |
| 67695 | 45225283 | 45230214 | 252200 |
| 67696 | 45239462 | 45247070 | |
| 67697 | 45247366 | 45254949 | 069399 |
| 67698 | 45255750 | 45258915 | 069399 |
| 67699 | 45268054 | 45269624 | 236807, 253027 |
| 67700 | 45276452 | 45281932 | 142273 |
| 67701 | 45299841 | 45300426 | 142273 |
| 67702 | 45310298 | 45310835 | |
| 67703 | 45311113 | 45312029 | |
| 67704 | 45347437 | 45351541 | 130202 |
| 67705 | 45393246 | 45395579 | 130204, 267282 |
| 67706 | 45406768 | 45408392 | 130204 |
| 67707 | 45408431 | 45410067 | 130203 |
| 67708 | 45413269 | 45413975 | |
| 67709 | 45414108 | 45414903 | |
| 67710 | 45416497 | 45419012 | 130208 |
| 67711 | 45427177 | 45428959 | |
| 67712 | 45433194 | 45434046 | 214855 |
| 67713 | 45438796 | 45439674 | |
| 67714 | 45442637 | 45443425 | |
| 67715 | 45445111 | 45446000 | 224916, 267467 |
| 67716 | 45446434 | 45451163 | 234906, 224916, 267467 |
| 67717 | 45457450 | 45460270 | 104853 |
| 67718 | 45504326 | 45505465 | 104856 |
| 67719 | 45542072 | 45542886 | 104859 |
| 67720 | 45548757 | 45549789 | 104859 |
| 67721 | 45578991 | 45580152 | 267348, 170684 |
| 67722 | 45581654 | 45583775 | 142252, 007047, 267348 |
| 67723 | 45594377 | 45597554 | 104866, 142252, 007047 |
| 67724 | 45600873 | 45601970 | 104866, 007047 |
| 67725 | 45680369 | 45683709 | 267545, 189114, 007255, |
| | | | 007047 |
| 67726 | 45753895 | 45755650 | 007047 |
| 67727 | 45776035 | 45777113 | 007047 |
| 67728 | 45782929 | 45784327 | 007047 |
| 67729 | 45792090 | 45792462 | 007047 |
| 67730 | 45793261 | 45794460 | 007047 |
| 67731 | 45797023 | 45798326 | 007047 |
| 67732 | 45873048 | 45874625 | 104884 |
| 67733 | 45879135 | 45880522 | |
| 67734 | 45901221 | 45902195 | 104881 |
| 67735 | 45907022 | 45910807 | 117877, 012061, 104881 |
| 67736 | 45926660 | 45929038 | 012061 |
| 67737 | 45942389 | 45944029 | 012061 |
| 67738 | 45946693 | 45948210 | 012061 |
| 67739 | 45953629 | 45955055 | 012061 |
| 67740 | 45956975 | 45960403 | 012061 |
| 67741 | 45970712 | 45971256 | 125740, 012061 |
| 67742 | 45971281 | 45972676 | 125740, 012061 |
| 67743 | 45979270 | 45982699 | 012061 |
| 67744 | 46009110 | 46013449 | 125753 |
| 67745 | 46017565 | 46018491 | 125753 |
| 67746 | 46021686 | 46022115 | 125753 |
| 67747 | 46037984 | 46038559 | 125741 |
| 67748 | 46071822 | 46073647 | 125741 |
| 67749 | 46079334 | 46081578 | 125741 |
| 67750 | 46086650 | 46089127 | 125741 |
| 67751 | 46110458 | 46111011 | 125746 |
| 67752 | 46141774 | 46143009 | 199066, 125746 |
| 67753 | 46144535 | 46146819 | 267757, 125746 |
| 67754 | 46194425 | 46196501 | 011478, 125743 |
| 67755 | 46219263 | 46221906 | 177051 |
| 67756 | 46223835 | 46224488 | 177051 |
| 67757 | 46233004 | 46234714 | 177051 |
| 67758 | 46270393 | 46274381 | 267395, 104936, 177045, |
| | | | 259605 |
| 67759 | 46284595 | 46285554 | 268434, 104936 |
| 67760 | 46295123 | 46296304 | 185800 |
| 67761 | 46365181 | 46366726 | 125755 |
| 67762 | 46366815 | 46368753 | 170608 |
| 67763 | 46376483 | 46377571 | 170608 |
| 67764 | 46379263 | 46383203 | |
| 67765 | 46384769 | 46387625 | 170604 |
| 67766 | 46388325 | 46390982 | 170604 |
| 67767 | 46405401 | 46406590 | 176182 |
| 67768 | 46498013 | 46499528 | 104983 |
| 67769 | 46634835 | 46637939 | 267922 |
| 67770 | 46692620 | 46694211 | 268621, 267922 |
| 67771 | 46696064 | 46697916 | 268621, 267922 |
| 67772 | 46849496 | 46851121 | 011485 |
| 67773 | 46857899 | 46860274 | 011485 |
| 67774 | 46906967 | 46908728 | |
| 67775 | 47003774 | 47004670 | 230510 |
| 67776 | 47049418 | 47050053 | 230510 |
| 67777 | 47103318 | 47107225 | 160014, 268423, 230510 |
| 67778 | 47108076 | 47108632 | 160014 |
| 67779 | 47141621 | 47142324 | |
| 67780 | 47216896 | 47218422 | 105287 |
| 67781 | 47219194 | 47221300 | 105287 |
| 67782 | 47248371 | 47249997 | 181027, 090372 |
| 67783 | 47353444 | 47354376 | 042753 |
| 67784 | 47359326 | 47360266 | |
| 67785 | 47362919 | 47366228 | |
| 67786 | 47378452 | 47379463 | |
| 67787 | 47388465 | 47389128 | |
| 67788 | 47392703 | 47395265 | |
| 67789 | 47427920 | 47429519 | 160007 |
| 67790 | 47435614 | 47436572 | 160007 |
| 67791 | 47446474 | 47447254 | 160007 |
| 67792 | 47448373 | 47449266 | 160007 |
| 67793 | 47450010 | 47450589 | 160007 |
| 67794 | 47453636 | 47455649 | 160007 |
| 67795 | 47478385 | 47480403 | 160007 |
| 67796 | 47487062 | 47489752 | 160007 |
| 67797 | 47496074 | 47496841 | 160007 |
| 67798 | 47516069 | 47516841 | |
| 67799 | 47538207 | 47539831 | 130751 |
| 67800 | 47551240 | 47552237 | 130748 |
| 67801 | 47561823 | 47562462 | |
| 67802 | 47582510 | 47583876 | 130749 |
| 67803 | 47598000 | 47600821 | 130749 |
| 67804 | 47608945 | 47612223 | 130749 |
| 67805 | 47613883 | 47617756 | 142230, 130749 |
| 67806 | 47633220 | 47634996 | 142230 |
| 67807 | 47728679 | 47732102 | 265134, 105327 |
| 67808 | 47732821 | 47736478 | 105327 |
| 67809 | 47743609 | 47744928 | |
| 67810 | 47746212 | 47748398 | |
| 67811 | 47758977 | 47761183 | 105321 |
| 67812 | 47792623 | 47793462 | 197405 |
| 67813 | 47808277 | 47809280 | 197405 |
| 67814 | 47817993 | 47819531 | 197405 |
| 67815 | 47852154 | 47853489 | 134815 |
| 67816 | 47879743 | 47880398 | 134815 |
| 67817 | 47986873 | 47988279 | 268061, 118162 |
| 67818 | 48015026 | 48019751 | 105402 |
| 67819 | 48025110 | 48025834 | 118156 |
| 67820 | 48101621 | 48102977 | |
| 67821 | 48103313 | 48105684 | |
| 67822 | 48110688 | 48114526 | 063169, 268746 |
| 67823 | 48121335 | 48123921 | 063169, 268746, 269806 |
| 67824 | 48150676 | 48152308 | 063169, 268746 |
| 67825 | 48162991 | 48168851 | 063169, 268746 |
| 67826 | 48245635 | 48247288 | 024422 |
| 67827 | 48248168 | 48249387 | 105373 |
| 67828 | 48260162 | 48261693 | 269656, 105373 |
| 67829 | 48281246 | 48282990 | 178980 |
| 67830 | 48291877 | 48292571 | |
| 67831 | 48387690 | 48390110 | 105398 |
| 67832 | 48394190 | 48397257 | |
| 67833 | 48397802 | 48400754 | |
| 67834 | 48610411 | 48611458 | 105499 |
| 67835 | 48613187 | 48614986 | 105499 |
| 67836 | 48673171 | 48674578 | 185453, 105486 |
| 67837 | 48763171 | 48763799 | 178150 |
| 67838 | 48824470 | 48826055 | 142227, 105479 |
| 67839 | 48828432 | 48829616 | 142227 |
| 67840 | 48833307 | 48834454 | 142227 |
| 67841 | 48866198 | 48867551 | 161558 |
| 67842 | 48893576 | 48895025 | 105438 |
| 67843 | 48948599 | 48950155 | 105447 |
| 67844 | 48971937 | 48973768 | 105443, 268530, 268465 |
| 67845 | 48990534 | 48992215 | 142235 |
| 67846 | 49061450 | 49062004 | 088002 |
| 67847 | 49087222 | 49087954 | 088002 |
| 67848 | 49091857 | 49092754 | 088002 |
| 67849 | 49121257 | 49123605 | 268093, 063176, 063177 |
| 67850 | 49127112 | 49127968 | 268093, 063176 |
| 67851 | 49130313 | 49131196 | 063176 |
| 67852 | 49136543 | 49137162 | 105516 |
| 67853 | 49137475 | 49140329 | 105516 |
| 67854 | 49140543 | 49141821 | 232871, 063180, 105516 |
| 67855 | 49217015 | 49218478 | 176909 |
| 67856 | 49257943 | 49259679 | 105550, 174951 |
| 67857 | 49272101 | 49274876 | |
| 67858 | 49274989 | 49276970 | |
| 67859 | 49298168 | 49299280 | 105552 |
| 67860 | 49309901 | 49311737 | 105552 |
| 67861 | 49313530 | 49314621 | 105552 |
| 67862 | 49315339 | 49316136 | |
| 67863 | 49319140 | 49320068 | 087076 |
| 67864 | 49322464 | 49323300 | 087076 |
| 67865 | 49329762 | 49330195 | 087076 |
| 67866 | 49370772 | 49372518 | 105559 |
| 67867 | 49375260 | 49379780 | 087074 |
| 67868 | 49402976 | 49404440 | 104805 |
| 67869 | 49410588 | 49412498 | 104805 |
| 67870 | 49457247 | 49459494 | 087088 |
| 67871 | 49463868 | 49472834 | 087086, 267898, 104812, |
| | | | 087088 |
| 67872 | 49481872 | 49482930 | 104812 |
| 67873 | 49495554 | 49497893 | 183207, 104812 |
| 67874 | 49588083 | 49589896 | 104852 |
| 67875 | 49621207 | 49622678 | 221916, 177380, 104863 |
| 67876 | 49648857 | 49649590 | 177380 |
| 67877 | 49652653 | 49654427 | 177380 |
| 67878 | 49660856 | 49661961 | 130529 |
| 67879 | 49713111 | 49714918 | 130529 |
| 67880 | 49842928 | 49843912 | 074219, 197813, 104894 |
| 67881 | 49945857 | 49947380 | |
| 67882 | 49954301 | 49955654 | 104872 |
| 67883 | 49977080 | 49978294 | 090554, 273189, 269469 |
| 67884 | 49989867 | 49992079 | 142541, 273189 |
| 67885 | 49998864 | 50000764 | 142534 |
| 67886 | 50016210 | 50017855 | 104870 |
| 67887 | 50083081 | 50085031 | 126460, 142546 |
| 67888 | 50093304 | 50095754 | 126464, 126460, 142546 |
| 67889 | 50142671 | 50146529 | 126461, 126458 |
| 67890 | 50168129 | 50170114 | 126453, 126456 |
| 67891 | 50179009 | 50182138 | 126457 |
| 67892 | 50268503 | 50271205 | 196961 |
| 67893 | 50274874 | 50275421 | 196961 |
| 67894 | 50316048 | 50317343 | 010361 |
| 67895 | 50353714 | 50355319 | 104960, 268006 |
| 67896 | 50370063 | 50371505 | 039650 |
| 67897 | 50372740 | 50373819 | 204673 |
| 67898 | 50378520 | 50381756 | 104946, 204673 |
| 67899 | 50425076 | 50428562 | 213024, 269179, 104951 |
| 67900 | 50430550 | 50433738 | 169136, 213024, 104951, |
| | | | 269179 |
| 67901 | 50435339 | 50436681 | 265438, 269179, 169136 |
| 67902 | 50507140 | 50508125 | 105053 |
| 67903 | 50526081 | 50526472 | 105053 |
| 67904 | 50527942 | 50529660 | 142528, 105053 |
| 67905 | 50650952 | 50651790 | |
| 67906 | 50708268 | 50709121 | 105357 |
| 67907 | 50737198 | 50738283 | 105357 |
| 67908 | 50744117 | 50744622 | 105357 |
| 67909 | 50828569 | 50829320 | 131398 |
| 67910 | 50831958 | 50833077 | 131408, 131398 |
| 67911 | 50836111 | 50837783 | 131401, 131398, 131408 |
| 67912 | 50860288 | 50861394 | 131408 |
| 67913 | 50871600 | 50873080 | 269392, 131408 |
| 67914 | 50879263 | 50880358 | 131408 |
| 67915 | 50979361 | 50980452 | 161671, 142530 |
| 67916 | 51013605 | 51015520 | 204653, 161677 |
| 67917 | 51016037 | 51017556 | 204653 |
| 67918 | 51306921 | 51308958 | 167747 |
| 67919 | 51610939 | 51611996 | 142544 |
| 67920 | 51841860 | 51842705 | 186806 |
| 67921 | 51842794 | 51844644 | 267905, 186806 |
| 67922 | 51850294 | 51851445 | 105379, 267984 |
| 67923 | 51853054 | 51855739 | 269403, 105379, 267984 |
| 67924 | 51859337 | 51860114 | 269403, 105379 |
| 67925 | 51865367 | 51872061 | 160318, 269403, 105379, |
| | | | 268520 |
| 67926 | 51897381 | 51897929 | 268889 |
| 67927 | 51977792 | 51979061 | |
| 67928 | 52073866 | 52075208 | 105497 |
| 67929 | 52097251 | 52098241 | 269388, 167765 |
| 67930 | 52407312 | 52408786 | 198093 |
| 67931 | 52429815 | 52431475 | 176024 |
| 67932 | 52489252 | 52490597 | 256683 |
| 67933 | 52511074 | 52512361 | 197619 |
| 67934 | 52530936 | 52532497 | 142556 |
| 67935 | 52551389 | 52553043 | 256087 |
| 67936 | 52597921 | 52599271 | 197608, 256087 |
| 67937 | 52641807 | 52643608 | 204611, 267827 |
| 67938 | 52673929 | 52675217 | 267927, 267827, 196267 |
| 67939 | 52692067 | 52694769 | 105568 |
| 67940 | 52772458 | 52774086 | 196214 |
| 67941 | 52799667 | 52801565 | 198464, 269102 |
| 67942 | 52900608 | 52901827 | 167555, 269834 |
| 67943 | 53030276 | 53031294 | 198482 |
| 67944 | 53605886 | 53607210 | 170949 |
| 67945 | 53700267 | 53700855 | 269051 |
| 67946 | 54367550 | 54368221 | 232324, 228323 |
| 67947 | 54370516 | 54373512 | 228323, 179820 |
| 67948 | 54605354 | 54606936 | 170906, 170909 |
| 67949 | 54617883 | 54619362 | 105618, 105619 |
| 67950 | 54640056 | 54642471 | 088038 |
| 67951 | 54662682 | 54663998 | 167608, 105617 |
| 67952 | 54666024 | 54666669 | 167608 |
| 67953 | 54693189 | 54695208 | 170892, 125505 |
| 67954 | 54703795 | 54705974 | 170889, 223660 |
| 67955 | 54706952 | 54707815 | 170889 |
| 67956 | 54711935 | 54712699 | 170889 |
| 67957 | 54712935 | 54714263 | 224579, 170889 |
| 67958 | 54959079 | 54961953 | 167615, 226696 |
| 67959 | 54972934 | 54976621 | 167617, 182909, 167615 |
| 67960 | 55573840 | 55574889 | 160439 |
| 67961 | 55627793 | 55630417 | 125503 |
| 67962 | 55717799 | 55719390 | 080031 |
| 67963 | 55720172 | 55721989 | 080031 |
| 67964 | 55727565 | 55728881 | 267649 |
| 67965 | 55739764 | 55740461 | 180089, 269808 |
| 67966 | 55768819 | 55771580 | 105063 |
| 67967 | 55790452 | 55791793 | 133265 |
| 67968 | 55838791 | 55839289 | 180061 |
| 67969 | 55845213 | 55845616 | 180061 |
| 67970 | 55849896 | 55852262 | 267531, 133247 |
| 67971 | 55894810 | 55898363 | 108107, 233493 |
| 67972 | 55918917 | 55920382 | 108106 |
| 67973 | 55971312 | 55973920 | 063241 |
| 67974 | 55978863 | 55979662 | |
| 67975 | 55986973 | 55988827 | 197483, 269859 |
| 67976 | 56056169 | 56056705 | 231274 |
| 67977 | 56064597 | 56065021 | |
| 67978 | 56091337 | 56093885 | 218891 |
| 67979 | 56110336 | 56113239 | 171443, 179943 |
| 67980 | 56116664 | 56117932 | 261221 |
| 67981 | 56135529 | 56136158 | 179922 |
| 67982 | 56145503 | 56147570 | 213015, 171425 |
| 67983 | 56151302 | 56155997 | 173581, 213015, 171425 |
| 67984 | 56158989 | 56160481 | 173581 |
| 67985 | 56164959 | 56167897 | 063244 |
| 67986 | 56186039 | 56187717 | 063245, 267523, 063244 |
| 67987 | 56194850 | 56195637 | 063245 |
| 67988 | 56582495 | 56582924 | 267522 |
| 67989 | 56596936 | 56597414 | |
| 67990 | 56616419 | 56617003 | 223060, 142409 |
| 67991 | 56621791 | 56622404 | 142409 |
| 67992 | 56630502 | 56633629 | 142409 |
| 67993 | 56648857 | 56650129 | 167685 |
| 67994 | 56651471 | 56654143 | 167685 |
| 67995 | 56710955 | 56712161 | |
| 67996 | 56825023 | 56827275 | 267549, 131848 |
| 67997 | 56879338 | 56880451 | 240225, 131848, 267606 |
| 67998 | 56904408 | 56905594 | 267454, 018869 |
| 67999 | 56914788 | 56916247 | 198440 |
| 68000 | 57078110 | 57079779 | 197016, 269696 |
| 68001 | 57348662 | 57351339 | 198300, 269699 |
| 68002 | 57702452 | 57703802 | 083844 |
| 68003 | 57751261 | 57753282 | 204524 |
| 68004 | 57790450 | 57792932 | 197714, 267871 |
| 68005 | 57831328 | 57832600 | 178229 |
| 68006 | 57862078 | 57863474 | 131845, 268678 |
| 68007 | 57874390 | 57875773 | 152433, 256060, 268133 |
| 68008 | 57900832 | 57902257 | 188785, 269533, 268133 |
| 68009 | 57922103 | 57923408 | 186272, 268533, 269533 |
| 68010 | 57945782 | 57947554 | 186230, 268533 |
| 68011 | 58089507 | 58090630 | 171649, 083817 |
| 68012 | 58110965 | 58111896 | 183647 |
| 68013 | 58125297 | 58126414 | 213762 |
| 68014 | 58144349 | 58145172 | 121417 |
| 68015 | 58192937 | 58194134 | 269026, 204519 |
| 68016 | 58257938 | 58259184 | 152443, 269026 |
| 68017 | 58280683 | 58282101 | 083828 |
| 68018 | 58314957 | 58315251 | 178935, 083828 |
| 68019 | 58325236 | 58326507 | 178935, 083828 |
| 68020 | 58330654 | 58331828 | 269343, 268750, 083828 |
| 68021 | 58513145 | 58514983 | 176593, 166704 |
| 68022 | 58661253 | 58662711 | 181894 |
| 68023 | 58665440 | 58666914 | 181894 |
| 68024 | 58693318 | 58695840 | 171606 |
| 68025 | 58739416 | 58740973 | 269545, 198131 |
| 68026 | 58789235 | 58791199 | 273439, 083842, 267216, |
| | | | 268516, 269545 |
| 68027 | 58816298 | 58817045 | 142396, 267216 |
| 68028 | 58837815 | 58839857 | 182318 |
| 68029 | 58861059 | 58862549 | 121410, 268230, 268895 |
| 68030 | 58863697 | 58865336 | 121410, 268230, 268895 |
| 68031 | 58872847 | 58874559 | 269054, 268230, 174586 |
| 68032 | 58883728 | 58884215 | 152475 |
| 68033 | 58891435 | 58892662 | 152475 |
| 68034 | 58896871 | 58899543 | 266640, 083845 |
| 68035 | 58912552 | 58913425 | 171574 |
| 68036 | 58918870 | 58921035 | 232098, 171574 |
| 68037 | 58962415 | 58963499 | 249471 |
| 68038 | 58977876 | 58979179 | 083812 |
| 68039 | 58986900 | 58988593 | 269106, 083838 |
| 68040 | 59010621 | 59011812 | 083807 |
| 68041 | 59021118 | 59023795 | 083807 |
| 68042 | 59024429 | 59026473 | 119574 |
| 68043 | 59030116 | 59032361 | 119574 |
| 68044 | 59054689 | 59057199 | 130726, 264910 |
| 68045 | 59065360 | 59067289 | 130725, 130724 |
| 68046 | 59068793 | 59071655 | 267858, 130725 |
| 68047 | 59073631 | 59074214 | 099326, 267858 |
| 68048 | 59083821 | 59085352 | 213753, 099326, 267858 |
| 68049 | 59086461 | 59087720 | 213753 |

The locations of ODSCs on Chromosome 20 are shown in Table 62.

TABLE 62
ODSC Locations on Chromosome 20

| SEQ ID NO. | Start | End | Overlapping Gene(s) |
|---|---|---|---|
| 68050 | 237954 | 238614 | 186458 |
| 68051 | 270691 | 271622 | 196476 |
| 68052 | 277620 | 280209 | 247315, 177764 |
| 68053 | 304385 | 307311 | 177732, 225377 |
| 68054 | 307405 | 308347 | 177732, 225377 |
| 68055 | 327794 | 329383 | 225377, 125841 |
| 68056 | 342603 | 345160 | |
| 68057 | 350233 | 354744 | |
| 68058 | 360096 | 364311 | 101255 |
| 68059 | 370831 | 371320 | 101255 |
| 68060 | 376080 | 378066 | 101255 |
| 68061 | 380578 | 384884 | |
| 68062 | 387017 | 391352 | 125826 |
| 68063 | 396003 | 396981 | 125826 |
| 68064 | 400093 | 401451 | 125826 |
| 68065 | 415099 | 415715 | |
| 68066 | 442003 | 443706 | 125875 |
| 68067 | 508762 | 510975 | 101266 |
| 68068 | 523217 | 525382 | 101266 |
| 68069 | 626716 | 627568 | 270299, 271303 |
| 68070 | 632305 | 635351 | 271303, 270299 |
| 68071 | 680707 | 681156 | |
| 68072 | 814025 | 814964 | 125898 |
| 68073 | 821659 | 822645 | 125898 |
| 68074 | 1086196 | 1087024 | |
| 68075 | 1098373 | 1100537 | 125818 |
| 68076 | 1103162 | 1104098 | 125818 |
| 68077 | 1130866 | 1132062 | 125818 |
| 68078 | 1165268 | 1166860 | 125895 |
| 68079 | 1305460 | 1310583 | 234684, 125775 |
| 68080 | 1372500 | 1374647 | 88832 |
| 68081 | 1380070 | 1380798 | |
| 68082 | 1381952 | 1382672 | |
| 68083 | 1396389 | 1398307 | |
| 68084 | 1445701 | 1447864 | 88833 |
| 68085 | 1449740 | 1451236 | 88833 |
| 68086 | 1757157 | 1758329 | 232528 |
| 68087 | 1789073 | 1794867 | 230839 |
| 68088 | 1796414 | 1797681 | 230839 |
| 68089 | 1807882 | 1810004 | |
| 68090 | 1835750 | 1836255 | |
| 68091 | 1854715 | 1855983 | |
| 68092 | 1861437 | 1862674 | |
| 68093 | 1871334 | 1871974 | |
| 68094 | 1874389 | 1877115 | 198053 |
| 68095 | 1900757 | 1901769 | 198053 |
| 68096 | 1916213 | 1917138 | 198053 |
| 68097 | 1919481 | 1920334 | 198053 |
| 68098 | 1923858 | 1926744 | |
| 68099 | 1927347 | 1928241 | 233896 |
| 68100 | 1946081 | 1946638 | 233896 |
| 68101 | 2047093 | 2047561 | |
| 68102 | 2061237 | 2062147 | |
| 68103 | 2082209 | 2084699 | 125834 |
| 68104 | 2217019 | 2218450 | |
| 68105 | 2241460 | 2242532 | |
| 68106 | 2263806 | 2266270 | |
| 68107 | 2279534 | 2283001 | 125780 |
| 68108 | 2284018 | 2284681 | 125780 |
| 68109 | 2332332 | 2333073 | |
| 68110 | 2450104 | 2452025 | 125835, 256566 |
| 68111 | 2488585 | 2490510 | 256566, 088876 |
| 68112 | 2504994 | 2506264 | 88876 |
| 68113 | 2631966 | 2634428 | 101361, 221062 |
| 68114 | 2644001 | 2645706 | 228293, 101365 |
| 68115 | 2731700 | 2732909 | 88881 |
| 68116 | 2820337 | 2822339 | 215305, 132635 |
| 68117 | 2853184 | 2855032 | 132670 |
| 68118 | 2927901 | 2928681 | 132670 |
| 68119 | 3025623 | 3027688 | 125901, 125787 |
| 68120 | 3047405 | 3048585 | |
| 68121 | 3048868 | 3050545 | |
| 68122 | 3051692 | 3053280 | 101405 |
| 68123 | 3061275 | 3062326 | |
| 68124 | 3098523 | 3098997 | 185019, 235958 |
| 68125 | 3139417 | 3141222 | 215251, 185019 |
| 68126 | 3153577 | 3155508 | 88899 |
| 68127 | 3183132 | 3185597 | 198171 |
| 68128 | 3189093 | 3191458 | 125877 |
| 68129 | 3336448 | 3337883 | 88854 |
| 68130 | 3387401 | 3389653 | 88854 |
| 68131 | 3439854 | 3440394 | |
| 68132 | 3450869 | 3453427 | 88812 |
| 68133 | 3453940 | 3455104 | 88812 |
| 68134 | 3459510 | 3461394 | 88812 |
| 68135 | 3490599 | 3491428 | 88812 |
| 68136 | 3502499 | 3503054 | 88812 |
| 68137 | 3522142 | 3523375 | 88812 |
| 68138 | 3524217 | 3525889 | 88812 |
| 68139 | 3527528 | 3529148 | 88812 |
| 68140 | 3531430 | 3534926 | 88812 |
| 68141 | 3609270 | 3609805 | 88812 |
| 68142 | 3613146 | 3613710 | 88812 |
| 68143 | 3748235 | 3749730 | 101220 |
| 68144 | 3766330 | 3767895 | 101224, 125817 |
| 68145 | 3775814 | 3777088 | 101224 |
| 68146 | 3778558 | 3779832 | 101224 |
| 68147 | 3783324 | 3784335 | 101224 |
| 68148 | 3786491 | 3787229 | 101224 |
| 68149 | 3787237 | 3789684 | |
| 68150 | 3791480 | 3793261 | |
| 68151 | 3800079 | 3802273 | 125843 |
| 68152 | 3826616 | 3828764 | 88888 |
| 68153 | 3868966 | 3871667 | 125779, 229539 |
| 68154 | 3913107 | 3914031 | 101236 |
| 68155 | 3988368 | 3989095 | 101236 |
| 68156 | 3995513 | 3996785 | 101236 |
| 68157 | 4065720 | 4066559 | |
| 68158 | 4111135 | 4112583 | 88826 |
| 68159 | 4114853 | 4117915 | 88826 |
| 68160 | 4128698 | 4130579 | 88826 |
| 68161 | 4151577 | 4153705 | 88826 |
| 68162 | 4666057 | 4668113 | 171867 |
| 68163 | 4897620 | 4898355 | 89057 |
| 68164 | 4905773 | 4908295 | 89057 |
| 68165 | 4941791 | 4943516 | 89057 |
| 68166 | 4947361 | 4949078 | 89057 |
| 68167 | 4953500 | 4955072 | 89057 |
| 68168 | 4980484 | 4982549 | 89057 |
| 68169 | 5045596 | 5047173 | |
| 68170 | 5053740 | 5054817 | |
| 68171 | 5092773 | 5094783 | 252367, 089063 |
| 68172 | 5099658 | 5101637 | 132646 |
| 68173 | 5106871 | 5108831 | 101290, 132646 |
| 68174 | 5130544 | 5131967 | 101290 |
| 68175 | 5133236 | 5134248 | 101290 |
| 68176 | 5136600 | 5138687 | 101290 |
| 68177 | 5164205 | 5165123 | 101290 |
| 68178 | 5192460 | 5193029 | |
| 68179 | 5590417 | 5592168 | 125772 |
| 68180 | 5699485 | 5700170 | |
| 68181 | 5705215 | 5705649 | |
| 68182 | 5730552 | 5732004 | 171984 |
| 68183 | 5759789 | 5760626 | 171984 |
| 68184 | 5784907 | 5785993 | 171984 |
| 68185 | 5930333 | 5931894 | 125885, 089195 |
| 68186 | 5986137 | 5989301 | 88766 |
| 68187 | 6009925 | 6010744 | 88766 |
| 68188 | 6054716 | 6057249 | 101311 |
| 68189 | 6061922 | 6062382 | 101311 |
| 68190 | 6103128 | 6104226 | 101311 |
| 68191 | 6115146 | 6115733 | |
| 68192 | 6142780 | 6143290 | |
| 68193 | 6144897 | 6146423 | |
| 68194 | 6446740 | 6447574 | 229876 |
| 68195 | 6571240 | 6572604 | |
| 68196 | 6592146 | 6595087 | |
| 68197 | 6604512 | 6605638 | |
| 68198 | 6612707 | 6613302 | |
| 68199 | 6677812 | 6679807 | |
| 68200 | 6682204 | 6687213 | |
| 68201 | 6687350 | 6693522 | |
| 68202 | 6720047 | 6720738 | |
| 68203 | 6747684 | 6750409 | 125845 |
| 68204 | 6809389 | 6810096 | |
| 68205 | 6956716 | 6957612 | |
| 68206 | 6972921 | 6976154 | |
| 68207 | 7127150 | 7129369 | 232271, 228888 |
| 68208 | 7131421 | 7133861 | 228888 |
| 68209 | 7244707 | 7245376 | |
| 68210 | 7368404 | 7368987 | |
| 68211 | 7521477 | 7522350 | |
| 68212 | 7565471 | 7566651 | |
| 68213 | 7846543 | 7848031 | |
| 68214 | 7860409 | 7861067 | |
| 68215 | 7903994 | 7907682 | 101323 |
| 68216 | 7916324 | 7921713 | 101323 |
| 68217 | 7960984 | 7962375 | 125827 |
| 68218 | 7968718 | 7969530 | 125827 |
| 68219 | 7999283 | 8000866 | 229766, 125827 |
| 68220 | 8112183 | 8113739 | 182621 |
| 68221 | 8125125 | 8126383 | 182621 |
| 68222 | 8135303 | 8138244 | 182621 |
| 68223 | 8736932 | 8737494 | 182621 |
| 68224 | 8790015 | 8790397 | 182621 |
| 68225 | 10015445 | 10016530 | 227906, 132623 |
| 68226 | 10073550 | 10074194 | 227906 |
| 68227 | 10090178 | 10090745 | 227906 |
| 68228 | 10091603 | 10092258 | 227906 |
| 68229 | 10152352 | 10153643 | 227906 |
| 68230 | 10155868 | 10156547 | 227906, 230506 |
| 68231 | 10166354 | 10167342 | 232448, 227906, 230506 |
| 68232 | 10197821 | 10199836 | 132639, 227906 |
| 68233 | 10308677 | 10310334 | 227906 |
| 68234 | 10340068 | 10340688 | 227906 |
| 68235 | 10347888 | 10352613 | 227906 |
| 68236 | 10383613 | 10384522 | 125863 |
| 68237 | 10398602 | 10401779 | 125863 |
| 68238 | 10413826 | 10416578 | 149346, 125863 |
| 68239 | 10428519 | 10430374 | 149346 |
| 68240 | 10444191 | 10444664 | 149346 |
| 68241 | 10518352 | 10519123 | 149346 |
| 68242 | 10520637 | 10522906 | 149346 |
| 68243 | 10536376 | 10537319 | 149346 |
| 68244 | 10546170 | 10547326 | 149346 |
| 68245 | 10571618 | 10574225 | 149346 |
| 68246 | 10585043 | 10585877 | 149346 |
| 68247 | 10652397 | 10656118 | 270792, 101384 |
| 68248 | 10689053 | 10690156 | 270792 |
| 68249 | 10691720 | 10692722 | 270792 |
| 68250 | 10836843 | 10837375 | 270792 |
| 68251 | 10837884 | 10838579 | 270792 |
| 68252 | 10934988 | 10935885 | 270792 |
| 68253 | 11176546 | 11177629 | |
| 68254 | 11188179 | 11189075 | |
| 68255 | 11197331 | 11198337 | |
| 68256 | 11247880 | 11248681 | 230990 |
| 68257 | 11656970 | 11657655 | |
| 68258 | 11666776 | 11669357 | 261411 |
| 68259 | 11871360 | 11872657 | 132640 |
| 68260 | 11963184 | 11963897 | |
| 68261 | 12296167 | 12296896 | 230437 |
| 68262 | 12307783 | 12308309 | |
| 68263 | 12897178 | 12897875 | 233048 |
| 68264 | 12958102 | 12958981 | |
| 68265 | 12989603 | 12991327 | 172296 |
| 68266 | 13009754 | 13011875 | 172296 |
| 68267 | 13026785 | 13027598 | 172296 |
| 68268 | 13200627 | 13203213 | 101230 |
| 68269 | 13211578 | 13212402 | 101230 |
| 68270 | 13218073 | 13219639 | 226263, 101230 |
| 68271 | 13223931 | 13225499 | 225956, 101230 |
| 68272 | 13225789 | 13226502 | 225956, 101230 |
| 68273 | 13247849 | 13248457 | 101230, 089123 |
| 68274 | 13252818 | 13253708 | 101230, 089123 |
| 68275 | 13271332 | 13272484 | 101230, 089123 |
| 68276 | 13287790 | 13288634 | 89123 |
| 68277 | 13311817 | 13312881 | 89123 |
| 68278 | 13315698 | 13320968 | 89123 |
| 68279 | 13398774 | 13399900 | 89123 |
| 68280 | 13403165 | 13405478 | 89123 |
| 68281 | 13433141 | 13434015 | 89123 |
| 68282 | 13435848 | 13436744 | 89123 |
| 68283 | 13472267 | 13473487 | 89123 |
| 68284 | 13505752 | 13506618 | 89123 |
| 68285 | 13618770 | 13620070 | 89123 |
| 68286 | 13638304 | 13639185 | |
| 68287 | 13661687 | 13662760 | |
| 68288 | 13674971 | 13675540 | |
| 68289 | 13764817 | 13766940 | 101247, 089048 |
| 68290 | 13768615 | 13769571 | 101247 |
| 68291 | 13780735 | 13781837 | 101247 |
| 68292 | 13975780 | 13977101 | 172264, 101251 |
| 68293 | 14123732 | 14124522 | 172264 |
| 68294 | 14314641 | 14318744 | 125848, 172264 |
| 68295 | 14322300 | 14322975 | 172264 |
| 68296 | 14386557 | 14388899 | 172264 |
| 68297 | 14390330 | 14393676 | 172264 |
| 68298 | 14561105 | 14561774 | 172264, 227927 |
| 68299 | 14589972 | 14590881 | 172264, 227927 |
| 68300 | 14653797 | 14654253 | 172264 |
| 68301 | 14692123 | 14694009 | 172264 |
| 68302 | 14697491 | 14698688 | 172264 |
| 68303 | 14859093 | 14861163 | 172264 |
| 68304 | 14936034 | 14937546 | 172264 |
| 68305 | 14958903 | 14961186 | 172264 |
| 68306 | 14964819 | 14968144 | 172264 |
| 68307 | 14972264 | 14973255 | 172264 |
| 68308 | 14973497 | 14974438 | 172264 |
| 68309 | 14978306 | 14979004 | 172264 |
| 68310 | 15301502 | 15302131 | 172264 |
| 68311 | 15322887 | 15325070 | 172264 |
| 68312 | 15466854 | 15468968 | 172264 |
| 68313 | 15634630 | 15635294 | 172264 |
| 68314 | 15652999 | 15654791 | 172264 |
| 68315 | 15910739 | 15912020 | 172264 |
| 68316 | 16079769 | 16080519 | |
| 68317 | 16081260 | 16082012 | |
| 68318 | 16103709 | 16107039 | |
| 68319 | 16349197 | 16349841 | 89177 |
| 68320 | 16439908 | 16440414 | 89177 |
| 68321 | 16455739 | 16457065 | 89177 |
| 68322 | 16508613 | 16509336 | 89177 |
| 68323 | 16521559 | 16522884 | 89177 |
| 68324 | 16552733 | 16554849 | 89177 |
| 68325 | 16555194 | 16556342 | |
| 68326 | 16560606 | 16561340 | |
| 68327 | 16588041 | 16589055 | |
| 68328 | 16709927 | 16711661 | 125870, 228809 |
| 68329 | 17511239 | 17512706 | 125864 |
| 68330 | 17522049 | 17522986 | 125864 |
| 68331 | 17538665 | 17540281 | 125864 |
| 68332 | 17543224 | 17544018 | 125864 |
| 68333 | 17548963 | 17552422 | 125868, 125864 |
| 68334 | 17607154 | 17607870 | 125844 |
| 68335 | 17657425 | 17658102 | 125844 |
| 68336 | 17660083 | 17663898 | 125844 |
| 68337 | 17691072 | 17691896 | 125888 |
| 68338 | 17702538 | 17703073 | 125888 |
| 68339 | 17730631 | 17731673 | |
| 68340 | 17776633 | 17777217 | |
| 68341 | 17839425 | 17840678 | |
| 68342 | 17844134 | 17844886 | |
| 68343 | 17855541 | 17856550 | |
| 68344 | 17876731 | 17878408 | |
| 68345 | 17891344 | 17892106 | |
| 68346 | 17905708 | 17906422 | |
| 68347 | 17947627 | 17951300 | 125871, 089006, 125850 |
| 68348 | 17966215 | 17966773 | 125850, 125871 |
| 68349 | 17982128 | 17982718 | 125850 |
| 68350 | 18117308 | 18120192 | 232838, 149474 |
| 68351 | 18124598 | 18126095 | 149474 |
| 68352 | 18268055 | 18270164 | 125846 |
| 68353 | 18447250 | 18448747 | 132664, 089091 |
| 68354 | 18477239 | 18478271 | 89050 |
| 68355 | 18484927 | 18485477 | 214612 |
| 68356 | 18487370 | 18489479 | 101310 |
| 68357 | 18521551 | 18525651 | 101310 |
| 68358 | 18547123 | 18548948 | 232388 |
| 68359 | 18565228 | 18566252 | |
| 68360 | 18568400 | 18569373 | 125821 |
| 68361 | 18774397 | 18775464 | 273148, 179935 |
| 68362 | 18837752 | 18840108 | |
| 68363 | 19673117 | 19673898 | 232675, 185052 |
| 68364 | 19737677 | 19740182 | 268628, 233895 |
| 68365 | 19740347 | 19740975 | 233895 |
| 68366 | 19783095 | 19783929 | 233895 |
| 68367 | 19865097 | 19867790 | 132669 |
| 68368 | 19944680 | 19945693 | 132669 |
| 68369 | 19990257 | 19992091 | |
| 68370 | 19997435 | 19998845 | 173418 |
| 68371 | 20032398 | 20033688 | 089101, 101343 |
| 68372 | 20437960 | 20438753 | 188559 |
| 68373 | 20461343 | 20462345 | 188559 |
| 68374 | 20473596 | 20474078 | 188559 |
| 68375 | 20499628 | 20500450 | 188559 |
| 68376 | 20679307 | 20680271 | 188559 |
| 68377 | 20691107 | 20694256 | 188559 |
| 68378 | 20712022 | 20713853 | |
| 68379 | 20714963 | 20716084 | |
| 68380 | 20723830 | 20725187 | |
| 68381 | 20736332 | 20737136 | |
| 68382 | 20739623 | 20740363 | |
| 68383 | 20760803 | 20761614 | |
| 68384 | 20762858 | 20763340 | |
| 68385 | 21105826 | 21106309 | |
| 68386 | 21106451 | 21107462 | 88970 |
| 68387 | 21182802 | 21183373 | 088970, 232712 |
| 68388 | 21183630 | 21184345 | 088970, 232712 |
| 68389 | 21282857 | 21284962 | 88930 |
| 68390 | 21485615 | 21487691 | |
| 68391 | 21514980 | 21515504 | |
| 68392 | 21544342 | 21546027 | |
| 68393 | 21961180 | 21964233 | |
| 68394 | 22391132 | 22394235 | 204684 |
| 68395 | 22399177 | 22399877 | 204684 |
| 68396 | 22406181 | 22407166 | |
| 68397 | 22409758 | 22411977 | |
| 68398 | 22432995 | 22434644 | |
| 68399 | 22441345 | 22441914 | |
| 68400 | 22443583 | 22444099 | |
| 68401 | 22445821 | 22447447 | |
| 68402 | 22451947 | 22452465 | |
| 68403 | 22469790 | 22471878 | |
| 68404 | 22487756 | 22488588 | |
| 68405 | 22492492 | 22493840 | |
| 68406 | 22502912 | 22508321 | |
| 68407 | 22548578 | 22553618 | 259974 |
| 68408 | 22555074 | 22561339 | 259974 |
| 68409 | 22562024 | 22566455 | 125798 |
| 68410 | 22567146 | 22568328 | 237396 |
| 68411 | 22660794 | 22661503 | 230400 |
| 68412 | 22672065 | 22672886 | |
| 68413 | 22755917 | 22757580 | |
| 68414 | 22775621 | 22778184 | |
| 68415 | 22911960 | 22912972 | |
| 68416 | 23329737 | 23332347 | 269407, 132661, 234832 |
| 68417 | 23338015 | 23339144 | 232645 |
| 68418 | 23341183 | 23344161 | 125812 |
| 68419 | 23400083 | 23402428 | 125814 |
| 68420 | 23416053 | 23417466 | |
| 68421 | 23426170 | 23426748 | |
| 68422 | 23602964 | 23605251 | |
| 68423 | 23615944 | 23616495 | 101439 |
| 68424 | 23617232 | 23618919 | 101439 |
| 68425 | 24015134 | 24015999 | |
| 68426 | 24082285 | 24085693 | |
| 68427 | 24125935 | 24127848 | |
| 68428 | 24129485 | 24131187 | |
| 68429 | 24208158 | 24208795 | |
| 68430 | 24216804 | 24217262 | |
| 68431 | 24735855 | 24736768 | |
| 68432 | 24933450 | 24934309 | 77984 |
| 68433 | 24967922 | 24970192 | 101474 |
| 68434 | 24972290 | 24974587 | 101474 |
| 68435 | 24985763 | 24986233 | |
| 68436 | 24992191 | 24992939 | 154930 |
| 68437 | 25020205 | 25021073 | 154930 |
| 68438 | 25025618 | 25026396 | 154930 |
| 68439 | 25035535 | 25036235 | 154930 |
| 68440 | 25036791 | 25039166 | 154930 |
| 68441 | 25045838 | 25046905 | |
| 68442 | 25057120 | 25058648 | 100987 |
| 68443 | 25175622 | 25177731 | 197586 |
| 68444 | 25203805 | 25204345 | 197586 |
| 68445 | 25206694 | 25209065 | 268302, 197586 |
| 68446 | 25213440 | 25214805 | |
| 68447 | 25216837 | 25219878 | |
| 68448 | 25227021 | 25229761 | 100994 |
| 68449 | 25236034 | 25236379 | 100994 |
| 68450 | 25236908 | 25237389 | 100994 |
| 68451 | 25289044 | 25292562 | 100997 |
| 68452 | 25318951 | 25319514 | 100997 |
| 68453 | 25370301 | 25372404 | 100997 |
| 68454 | 25387922 | 25388811 | 101003 |
| 68455 | 25487554 | 25489978 | 101004 |
| 68456 | 25519454 | 25521754 | 101004 |
| 68457 | 25522410 | 25523476 | 101004 |
| 68458 | 25565250 | 25566753 | 101004 |
| 68459 | 25603192 | 25605820 | 213742, 170191 |
| 68460 | 25676055 | 25677969 | 130684 |
| 68461 | 26188669 | 26189540 | 227195 |
| 68462 | 26189742 | 26190651 | 227195 |
| 68463 | 29611374 | 29612474 | 149531 |
| 68464 | 30048289 | 30048709 | |
| 68465 | 30101888 | 30103556 | 101294 |
| 68466 | 30147091 | 30149745 | 101294 |
| 68467 | 30159831 | 30161978 | 230613 |
| 68468 | 30191764 | 30195294 | 264395, 125968 |
| 68469 | 30195451 | 30196827 | |
| 68470 | 30198239 | 30199217 | |
| 68471 | 30200456 | 30201105 | |
| 68472 | 30249165 | 30250610 | |
| 68473 | 30251379 | 30252507 | 171552 |
| 68474 | 30262513 | 30263347 | 171552 |
| 68475 | 30265337 | 30267459 | 171552 |
| 68476 | 30281533 | 30290216 | 171552, 236559 |
| 68477 | 30291993 | 30295307 | 171552, 236559 |
| 68478 | 30295485 | 30296771 | 171552, 236559 |
| 68479 | 30298172 | 30302503 | 171552, 236559 |
| 68480 | 30306372 | 30312124 | 268457, 171552 |
| 68481 | 30326305 | 30327205 | 88325 |
| 68482 | 30467277 | 30468285 | 131044 |
| 68483 | 30538712 | 30540197 | 88356 |
| 68484 | 30575167 | 30576145 | 260903 |
| 68485 | 30696768 | 30698642 | 101337 |
| 68486 | 30742369 | 30743581 | 101337 |
| 68487 | 30794315 | 30796380 | 101346, 126003 |
| 68488 | 30804116 | 30805285 | 101346 |
| 68489 | 30815360 | 30816249 | 101346 |
| 68490 | 30823454 | 30824107 | 101346 |
| 68491 | 30864946 | 30866356 | 101350, 264308 |
| 68492 | 30877280 | 30878310 | 101350 |
| 68493 | 30913335 | 30915257 | 101350 |
| 68494 | 30942154 | 30947738 | 171456 |
| 68495 | 31025424 | 31026023 | 171456 |
| 68496 | 31026546 | 31028813 | 171456 |
| 68497 | 31052763 | 31054722 | 197183 |
| 68498 | 31060774 | 31062030 | 197183 |
| 68499 | 31070256 | 31073985 | 197183 |
| 68500 | 31103409 | 31104087 | 197183 |
| 68501 | 31266651 | 31267306 | |
| 68502 | 31273561 | 31274074 | |
| 68503 | 31296734 | 31297820 | 149600 |
| 68504 | 31318985 | 31320201 | 149600 |
| 68505 | 31330320 | 31332415 | 149600 |
| 68506 | 31340057 | 31340979 | |
| 68507 | 31349879 | 31351256 | 88305 |
| 68508 | 31364911 | 31366030 | 88305 |
| 68509 | 31407243 | 31408852 | 101367 |
| 68510 | 31415733 | 31416547 | 101367 |
| 68511 | 31715497 | 31716135 | |
| 68512 | 31766597 | 31766942 | 131050 |
| 68513 | 31960871 | 31962730 | 101391 |
| 68514 | 31988618 | 31989698 | 101391 |
| 68515 | 32030713 | 32032252 | 101400 |
| 68516 | 32076962 | 32078863 | 78699 |
| 68517 | 32142533 | 32143343 | 78699 |
| 68518 | 32149804 | 32150560 | 78699 |
| 68519 | 32250735 | 32252451 | 149609, 125967 |
| 68520 | 32260853 | 32263038 | 271803, 125967 |
| 68521 | 32307181 | 32308527 | 101417 |
| 68522 | 32319207 | 32320827 | 131061, 229188 |
| 68523 | 32321410 | 32322232 | 131061 |
| 68524 | 32365610 | 32367323 | 131061 |
| 68525 | 32376848 | 32377869 | 230753, 131061 |
| 68526 | 32398767 | 32401471 | 101421, 230753 |
| 68527 | 32580168 | 32582983 | 125970, 228265 |
| 68528 | 32670590 | 32671286 | 125970 |
| 68529 | 32698652 | 32701037 | 125977 |
| 68530 | 32803629 | 32804458 | 214185, 101440 |
| 68531 | 32849905 | 32851302 | 250917, 101440 |
| 68532 | 32889850 | 32891717 | 101444 |
| 68533 | 32898129 | 32900980 | 101444 |
| 68534 | 32902244 | 32902720 | |
| 68535 | 32918744 | 32919448 | |
| 68536 | 32950738 | 32952506 | 78747 |
| 68537 | 32954621 | 32955669 | 78747 |
| 68538 | 32978531 | 32979595 | 78747 |
| 68539 | 32990374 | 32991482 | 234933, 078747 |
| 68540 | 32995558 | 32996827 | 78747 |
| 68541 | 33103060 | 33105346 | 125971 |
| 68542 | 33112698 | 33114297 | 125971 |
| 68543 | 33126384 | 33127035 | 125971 |
| 68544 | 33141078 | 33141763 | 101460 |
| 68545 | 33145668 | 33147742 | 101460 |
| 68546 | 33209428 | 33209898 | 101464 |
| 68547 | 33225151 | 33225799 | 101464 |
| 68548 | 33227656 | 33228163 | 101464 |
| 68549 | 33264206 | 33265386 | 101464 |
| 68550 | 33291130 | 33293924 | 078804, 198646 |
| 68551 | 33296314 | 33298118 | 078804, 198646 |
| 68552 | 33412339 | 33414238 | 198646 |
| 68553 | 33460023 | 33460990 | 131067, 131069 |
| 68554 | 33461719 | 33465403 | 131069 |
| 68555 | 33483883 | 33484299 | 131069 |
| 68556 | 33491704 | 33492565 | 131069 |
| 68557 | 33495377 | 33496035 | 131069 |
| 68558 | 33502567 | 33503371 | 131069 |
| 68559 | 33510392 | 33511571 | 131069 |
| 68560 | 33535758 | 33536303 | 100983 |
| 68561 | 33542240 | 33544123 | 100983 |
| 68562 | 33582297 | 33582933 | 78814 |
| 68563 | 33596657 | 33597158 | 100991 |
| 68564 | 33679375 | 33681840 | 100991 |
| 68565 | 33733908 | 33735942 | 88298 |
| 68566 | 33761597 | 33763164 | 088298, 101000 |
| 68567 | 33767193 | 33768195 | 88298 |
| 68568 | 33795689 | 33796729 | 88298 |
| 68569 | 33845273 | 33845837 | 088298, 125966, 126005 |
| 68570 | 33850716 | 33852907 | 088298, 125966, 126005 |
| 68571 | 33856751 | 33857523 | 088298, 125966, 261582, |
| | | | 126005 |
| 68572 | 33863412 | 33866406 | 088298, 125966, 126005, |
| | | | 261582 |
| 68573 | 33871232 | 33875336 | 125998, 235214, 242372 |
| 68574 | 33891228 | 33898317 | 101019 |
| 68575 | 33898564 | 33901435 | 101019 |
| 68576 | 33904884 | 33909180 | 101019 |
| 68577 | 33913266 | 33917054 | 101019 |
| 68578 | 33917084 | 33917694 | 101019 |
| 68579 | 33929167 | 33933994 | 101019 |
| 68580 | 33975145 | 33975681 | 101019 |
| 68581 | 33998827 | 34000657 | 101019 |
| 68582 | 34034546 | 34035433 | 125965 |
| 68583 | 34042156 | 34043915 | 126001, 125965 |
| 68584 | 34129632 | 34130518 | 125991 |
| 68585 | 34203374 | 34205841 | 61656 |
| 68586 | 34251459 | 34253516 | 214078, 244462, 272897 |
| 68587 | 34282864 | 34283717 | 244005 |
| 68588 | 34286543 | 34288091 | 125995, 244005 |
| 68589 | 34327811 | 34332499 | 131051 |
| 68590 | 34359554 | 34361008 | 25293 |
| 68591 | 34535508 | 34535979 | 25293 |
| 68592 | 34540666 | 34543429 | 171222 |
| 68593 | 34638315 | 34639710 | 260032 |
| 68594 | 34652455 | 34653833 | |
| 68595 | 34673141 | 34674151 | 234139 |
| 68596 | 34823766 | 34825500 | 131043 |
| 68597 | 35082932 | 35083847 | 80845 |
| 68598 | 35088688 | 35092069 | 80845 |
| 68599 | 35093260 | 35094332 | 80845 |
| 68600 | 35096735 | 35097879 | 80845 |
| 68601 | 35109178 | 35111337 | 80845 |
| 68602 | 35126914 | 35127584 | 80845 |
| 68603 | 35184838 | 35185347 | 259716, 232907 |
| 68604 | 35200277 | 35204200 | 118707, 259399, 232907, |
| | | | 259716 |
| 68605 | 35233262 | 35237150 | 101084, 259399 |
| 68606 | 35373033 | 35375211 | 265441, 101079 |
| 68607 | 35382894 | 35383520 | 149636 |
| 68608 | 35401406 | 35403163 | 149636 |
| 68609 | 35436998 | 35438209 | 149639 |
| 68610 | 35463584 | 35464285 | 149639 |
| 68611 | 35575045 | 35575882 | 101347 |
| 68612 | 35578930 | 35580697 | 101347 |
| 68613 | 35583071 | 35584254 | |
| 68614 | 35723544 | 35724904 | 269846, 080839 |
| 68615 | 35806532 | 35808953 | 118705, 101353 |
| 68616 | 35830633 | 35831172 | 118705 |
| 68617 | 35917326 | 35919073 | 101363 |
| 68618 | 35922294 | 35924645 | 101363 |
| 68619 | 35972782 | 35974946 | 197122 |
| 68620 | 36008526 | 36009284 | 197122 |
| 68621 | 36010298 | 36010850 | 197122 |
| 68622 | 36027097 | 36028274 | 197122 |
| 68623 | 36147532 | 36148528 | 166619 |
| 68624 | 36154630 | 36157671 | 166619 |
| 68625 | 36306026 | 36307142 | 204117 |
| 68626 | 36321879 | 36323268 | 132792 |
| 68627 | 36356021 | 36356766 | 132792 |
| 68628 | 36426616 | 36427360 | 132792 |
| 68629 | 36530405 | 36532146 | 132821 |
| 68630 | 36614113 | 36616647 | 101407 |
| 68631 | 36660786 | 36663477 | 101413, 101407 |
| 68632 | 36677900 | 36679857 | 101413 |
| 68633 | 36687375 | 36689599 | 101413 |
| 68634 | 36695151 | 36696358 | 101413 |
| 68635 | 36711232 | 36711785 | 101413 |
| 68636 | 36721955 | 36724380 | |
| 68637 | 36736833 | 36739904 | |
| 68638 | 36753345 | 36755969 | |
| 68639 | 36770862 | 36774599 | 198959 |
| 68640 | 36777946 | 36783241 | 198959 |
| 68641 | 36790308 | 36791187 | 198959 |
| 68642 | 36792466 | 36794167 | 198959 |
| 68643 | 36795517 | 36798654 | |
| 68644 | 36820097 | 36820470 | |
| 68645 | 36874859 | 36875501 | 149633 |
| 68646 | 36919270 | 36920123 | 206249, 101425 |
| 68647 | 36958785 | 36959653 | 101425 |
| 68648 | 36970997 | 36973471 | |
| 68649 | 36973659 | 36976744 | 129988 |
| 68650 | 36981184 | 36984379 | 129988 |
| 68651 | 36986175 | 36987167 | 129988 |
| 68652 | 37023713 | 37024397 | |
| 68653 | 37062686 | 37064267 | 196756 |
| 68654 | 37074821 | 37076322 | 174365 |
| 68655 | 37076475 | 37077434 | 174365 |
| 68656 | 37088815 | 37089296 | |
| 68657 | 37100845 | 37102392 | 170471 |
| 68658 | 37228248 | 37228670 | |
| 68659 | 37376858 | 37377824 | 101442 |
| 68660 | 37393632 | 37394002 | 101442 |
| 68661 | 37502947 | 37503548 | 101445 |
| 68662 | 37541924 | 37543273 | 101445 |
| 68663 | 37554491 | 37555652 | 101447 |
| 68664 | 37590138 | 37591862 | 261431, 101452 |
| 68665 | 37613834 | 37614906 | 101452 |
| 68666 | 37677904 | 37679136 | |
| 68667 | 37746040 | 37746620 | |
| 68668 | 37818751 | 37820301 | |
| 68669 | 37836926 | 37837861 | |
| 68670 | 37893077 | 37893626 | |
| 68671 | 38260074 | 38260642 | |
| 68672 | 38506602 | 38507251 | |
| 68673 | 38516969 | 38517452 | |
| 68674 | 38519077 | 38519764 | |
| 68675 | 38611798 | 38613004 | |
| 68676 | 38614297 | 38616057 | |
| 68677 | 38617235 | 38618289 | |
| 68678 | 38636151 | 38639245 | 237767 |
| 68679 | 39135026 | 39135755 | |
| 68680 | 39140105 | 39140626 | |
| 68681 | 39179507 | 39180594 | |
| 68682 | 39283284 | 39284577 | |
| 68683 | 39296787 | 39298562 | |
| 68684 | 39310653 | 39311282 | |
| 68685 | 39315816 | 39316771 | 204103 |
| 68686 | 39317297 | 39319786 | 204103 |
| 68687 | 39656868 | 39659022 | 198900 |
| 68688 | 39667559 | 39668245 | 198900 |
| 68689 | 39706801 | 39707440 | 198900 |
| 68690 | 39739552 | 39740187 | 198900, 226648 |
| 68691 | 39765306 | 39767102 | 124181, 226648 |
| 68692 | 39876530 | 39878991 | 174306 |
| 68693 | 39915195 | 39916911 | 174306 |
| 68694 | 39924407 | 39925599 | 174306 |
| 68695 | 39944624 | 39947595 | 174306 |
| 68696 | 39968403 | 39970193 | 132793 |
| 68697 | 40141211 | 40141655 | 124177 |
| 68698 | 40205400 | 40206766 | 124177 |
| 68699 | 40245904 | 40247606 | 124177 |
| 68700 | 40321171 | 40323419 | |
| 68701 | 41530363 | 41531264 | 196090 |
| 68702 | 42085545 | 42088154 | 124193 |
| 68703 | 42167500 | 42168307 | 185513 |
| 68704 | 42178862 | 42179564 | 185513, 226143 |
| 68705 | 42184905 | 42185727 | |
| 68706 | 42185974 | 42189587 | 101049 |
| 68707 | 42218777 | 42219802 | 101052 |
| 68708 | 42281413 | 42282201 | 215467 |
| 68709 | 42285370 | 42287033 | |
| 68710 | 42528488 | 42529475 | |
| 68711 | 42587442 | 42588829 | 124191 |
| 68712 | 42591786 | 42592458 | 124191 |
| 68713 | 42815838 | 42817128 | 149596 |
| 68714 | 42832737 | 42833803 | 132823 |
| 68715 | 42837644 | 42840733 | 223891, 132823 |
| 68716 | 42930318 | 42931753 | 197296 |
| 68717 | 42938788 | 42940295 | 197296 |
| 68718 | 42983595 | 42985418 | 101076, 226812 |
| 68719 | 42997914 | 43000814 | 101076 |
| 68720 | 43017004 | 43018060 | 101076, 229005 |
| 68721 | 43021214 | 43022266 | 101076, 229005 |
| 68722 | 43022829 | 43024534 | 229005, 101076 |
| 68723 | 43028850 | 43032375 | 101076 |
| 68724 | 43037289 | 43040412 | 101076 |
| 68725 | 43047762 | 43048652 | 101076 |
| 68726 | 43050726 | 43051237 | 101076 |
| 68727 | 43062147 | 43063780 | 273283 |
| 68728 | 43064037 | 43065261 | |
| 68729 | 43071174 | 43071656 | |
| 68730 | 43076304 | 43077147 | |
| 68731 | 43078528 | 43080208 | 237907 |
| 68732 | 43089385 | 43091233 | 168746 |
| 68733 | 43092960 | 43097684 | 226243, 168746 |
| 68734 | 43099786 | 43100533 | |
| 68735 | 43103736 | 43106314 | 124120 |
| 68736 | 43108290 | 43109157 | 124120 |
| 68737 | 43110309 | 43110952 | 124120 |
| 68738 | 43123006 | 43127033 | 132824, 124120 |
| 68739 | 43149533 | 43151288 | 132824 |
| 68740 | 43159890 | 43161281 | 168734 |
| 68741 | 43223088 | 43224029 | 168734 |
| 68742 | 43237480 | 43239493 | 168734, 269605 |
| 68743 | 43270803 | 43271694 | 196839 |
| 68744 | 43279789 | 43281391 | 196839 |
| 68745 | 43313044 | 43314410 | 132832 |
| 68746 | 43321274 | 43322229 | 132832 |
| 68747 | 43513820 | 43515666 | 166913 |
| 68748 | 43539980 | 43540433 | 101104 |
| 68749 | 43588217 | 43589400 | 25772 |
| 68750 | 43594363 | 43595633 | 101109, 227477 |
| 68751 | 43706137 | 43706794 | 101109 |
| 68752 | 43882771 | 43884202 | 124107 |
| 68753 | 43951432 | 43952237 | |
| 68754 | 43952754 | 43954764 | 124145 |
| 68755 | 43957029 | 43959530 | 124145 |
| 68756 | 43963942 | 43977782 | 124145 |
| 68757 | 43983388 | 43984290 | |
| 68758 | 43985889 | 43992672 | 254806, 204070 |
| 68759 | 44001931 | 44002918 | 124251, 254806, 204070 |
| 68760 | 44034785 | 44035738 | 244274, 254806, 124251 |
| 68761 | 44044178 | 44045680 | 124155 |
| 68762 | 44064943 | 44066150 | |
| 68763 | 44077637 | 44079436 | |
| 68764 | 44138910 | 44139815 | |
| 68765 | 44399600 | 44400304 | 124116 |
| 68766 | 44420205 | 44421432 | 101457, 124116 |
| 68767 | 44461569 | 44463325 | 124104, 101470 |
| 68768 | 44485141 | 44486941 | 132801, 101473 |
| 68769 | 44509665 | 44510527 | 168612 |
| 68770 | 44518561 | 44521124 | 064601, 124257 |
| 68771 | 44533863 | 44535066 | 100979 |
| 68772 | 44562245 | 44564769 | 100982 |
| 68773 | 44571822 | 44576154 | 100982 |
| 68774 | 44599442 | 44601518 | 198026 |
| 68775 | 44717278 | 44718880 | 124160 |
| 68776 | 44746307 | 44747615 | 101017 |
| 68777 | 44889079 | 44889495 | 149654 |
| 68778 | 44991998 | 44993374 | 80189 |
| 68779 | 45028861 | 45029288 | 62598 |
| 68780 | 45034358 | 45035991 | 62598 |
| 68781 | 45040247 | 45040792 | 62598 |
| 68782 | 45259714 | 45261088 | 158296 |
| 68783 | 45269094 | 45270003 | 158296 |
| 68784 | 45295441 | 45296244 | 158296 |
| 68785 | 45299386 | 45300029 | 158296 |
| 68786 | 45312670 | 45313370 | 172315 |
| 68787 | 45316910 | 45319156 | 271784, 172315 |
| 68788 | 45331224 | 45332836 | |
| 68789 | 45333422 | 45334863 | |
| 68790 | 45337802 | 45339566 | 197496 |
| 68791 | 45343804 | 45344401 | 197496 |
| 68792 | 45443378 | 45444494 | |
| 68793 | 45451580 | 45452987 | |
| 68794 | 45587673 | 45588226 | 64655 |
| 68795 | 45661569 | 45662272 | 64655 |
| 68796 | 45664626 | 45665264 | 64655 |
| 68797 | 45824803 | 45825522 | |
| 68798 | 45829024 | 45829771 | |
| 68799 | 45902060 | 45904282 | 101040 |
| 68800 | 45943083 | 45949320 | 267882, 263770, 101040 |
| 68801 | 45949400 | 45950692 | 267882, 101040 |
| 68802 | 45956773 | 45958079 | 101040 |
| 68803 | 45959358 | 45961081 | 101040 |
| 68804 | 45961277 | 45964573 | 101040 |
| 68805 | 45966025 | 45967548 | 101040 |
| 68806 | 45969487 | 45970050 | 101040 |
| 68807 | 45980986 | 45981870 | 231119, 101040 |
| 68808 | 45983276 | 45989920 | 231119, 101040 |
| 68809 | 46012430 | 46013404 | |
| 68810 | 46092628 | 46094617 | |
| 68811 | 46121745 | 46122801 | 201742 |
| 68812 | 46129660 | 46131954 | 124151 |
| 68813 | 46333475 | 46334313 | 196562 |
| 68814 | 46339092 | 46340677 | 196562 |
| 68815 | 46341880 | 46343407 | 196562 |
| 68816 | 46353920 | 46354576 | 196562 |
| 68817 | 46390972 | 46392675 | 196562 |
| 68818 | 46394440 | 46395629 | 196562 |
| 68819 | 46397088 | 46397730 | 196562 |
| 68820 | 46413072 | 46415796 | 196562 |
| 68821 | 46911762 | 46912974 | |
| 68822 | 46913933 | 46916841 | |
| 68823 | 46917428 | 46918501 | |
| 68824 | 46923560 | 46925224 | |
| 68825 | 47298960 | 47299550 | 124126 |
| 68826 | 47314223 | 47315388 | 124126 |
| 68827 | 47339288 | 47340350 | 124126 |
| 68828 | 47407467 | 47408470 | 124126 |
| 68829 | 47444489 | 47445501 | |
| 68830 | 47448037 | 47448964 | |
| 68831 | 47466833 | 47467764 | |
| 68832 | 47470309 | 47470902 | |
| 68833 | 47536690 | 47537148 | |
| 68834 | 47537450 | 47540444 | 124198 |
| 68835 | 47547880 | 47549707 | 124198 |
| 68836 | 47599890 | 47600350 | 124198 |
| 68837 | 47625514 | 47626232 | 124198 |
| 68838 | 47661776 | 47664297 | 124207, 227431 |
| 68839 | 47795632 | 47796871 | 124214 |
| 68840 | 47801773 | 47802573 | 124214 |
| 68841 | 47803370 | 47805333 | 124214 |
| 68842 | 47835506 | 47836893 | 124228 |
| 68843 | 47892809 | 47896377 | 209042, 177410, 124201 |
| 68844 | 47904394 | 47904914 | 177410 |
| 68845 | 47973055 | 47973744 | |
| 68846 | 47976779 | 47977433 | |
| 68847 | 47998055 | 47998621 | 158445 |
| 68848 | 48003333 | 48004019 | 158445 |
| 68849 | 48060941 | 48062422 | 158445 |
| 68850 | 48077345 | 48078025 | 158445 |
| 68851 | 48091645 | 48092902 | 158445 |
| 68852 | 48095267 | 48095760 | 158445 |
| 68853 | 48095817 | 48096928 | 158445 |
| 68854 | 48100183 | 48101279 | |
| 68855 | 48107095 | 48107836 | |
| 68856 | 48199932 | 48201642 | |
| 68857 | 48221040 | 48221472 | |
| 68858 | 48231832 | 48233035 | |
| 68859 | 48272750 | 48273454 | 158470 |
| 68860 | 48290627 | 48294295 | 158470 |
| 68861 | 48304326 | 48305344 | 158470 |
| 68862 | 48306986 | 48309145 | 158470 |
| 68863 | 48313234 | 48314678 | 158470 |
| 68864 | 48325499 | 48326626 | 158470 |
| 68865 | 48328030 | 48330840 | 158470 |
| 68866 | 48364193 | 48365335 | |
| 68867 | 48384583 | 48385625 | |
| 68868 | 48388573 | 48389400 | |
| 68869 | 48391221 | 48393864 | |
| 68870 | 48428370 | 48430740 | 197818 |
| 68871 | 48439652 | 48440791 | 197818 |
| 68872 | 48441938 | 48442811 | 197818 |
| 68873 | 48448677 | 48449600 | 197818 |
| 68874 | 48482426 | 48483310 | 197818 |
| 68875 | 48512683 | 48513186 | |
| 68876 | 48530817 | 48534627 | 158480 |
| 68877 | 48545092 | 48545662 | |
| 68878 | 48551354 | 48556186 | 124226 |
| 68879 | 48586335 | 48586854 | |
| 68880 | 48592785 | 48594134 | |
| 68881 | 48598486 | 48599682 | 124216 |
| 68882 | 48645603 | 48647541 | |
| 68883 | 48728376 | 48730666 | 124208, 240849, 244687 |
| 68884 | 48737246 | 48738120 | 124208, 240849 |
| 68885 | 48742547 | 48743743 | 124208, 240849 |
| 68886 | 48753051 | 48756488 | 124208, 240849 |
| 68887 | 48759798 | 48761959 | 124208, 240849 |
| 68888 | 48768700 | 48771108 | 124208, 240849 |
| 68889 | 48781881 | 48783486 | 234698, 237595 |
| 68890 | 48788372 | 48789855 | 231742 |
| 68891 | 48802597 | 48813127 | 172216 |
| 68892 | 48837880 | 48838587 | |
| 68893 | 48864080 | 48865442 | |
| 68894 | 48865920 | 48866948 | |
| 68895 | 48889641 | 48890702 | 224397 |
| 68896 | 48894757 | 48896073 | 224397 |
| 68897 | 48899601 | 48902593 | |
| 68898 | 48907990 | 48911881 | 203999 |
| 68899 | 48919940 | 48927058 | 203999 |
| 68900 | 48928731 | 48930316 | 203999, 233077 |
| 68901 | 48937164 | 48938086 | 233077 |
| 68902 | 48948314 | 48948787 | |
| 68903 | 48988340 | 48989663 | |
| 68904 | 48995666 | 48996635 | |
| 68905 | 49005308 | 49007253 | |
| 68906 | 49011318 | 49012913 | |
| 68907 | 49014492 | 49018403 | |
| 68908 | 49020092 | 49021401 | |
| 68909 | 49028438 | 49029134 | |
| 68910 | 49031088 | 49033842 | |
| 68911 | 49037907 | 49041337 | |
| 68912 | 49055057 | 49058863 | |
| 68913 | 49067460 | 49068315 | |
| 68914 | 49125959 | 49128052 | 196396 |
| 68915 | 49179699 | 49180893 | 196396 |
| 68916 | 49193625 | 49194184 | 196396, 232043 |
| 68917 | 49241063 | 49241646 | 42062 |
| 68918 | 49261753 | 49262667 | 234693, 042062 |
| 68919 | 49271793 | 49272514 | 042062, 234693 |
| 68920 | 49275307 | 49275804 | 042062, 234693 |
| 68921 | 49279465 | 49280451 | 42062 |
| 68922 | 49286934 | 49287401 | 42062 |
| 68923 | 49292295 | 49292924 | 42062 |
| 68924 | 49296399 | 49297474 | 42062 |
| 68925 | 49323964 | 49324683 | |
| 68926 | 49344100 | 49346602 | |
| 68927 | 49347567 | 49349565 | 124171 |
| 68928 | 49360331 | 49361893 | 124171 |
| 68929 | 49362744 | 49363619 | 124171 |
| 68930 | 49377508 | 49381017 | |
| 68931 | 49406903 | 49408125 | |
| 68932 | 49410876 | 49412035 | 124243 |
| 68933 | 49472527 | 49473577 | 124243 |
| 68934 | 49545809 | 49549001 | 259456, 101126 |
| 68935 | 49573797 | 49576433 | 124217, 000419 |
| 68936 | 49595818 | 49599261 | |
| 68937 | 49600445 | 49601202 | |
| 68938 | 50028846 | 50030074 | 101096 |
| 68939 | 50046595 | 50047595 | 101096 |
| 68940 | 50061938 | 50065138 | 101096 |
| 68941 | 50073013 | 50073982 | 101096 |
| 68942 | 50157151 | 50159250 | 101096 |
| 68943 | 50179865 | 50180956 | |
| 68944 | 50211322 | 50212070 | |
| 68945 | 50276611 | 50279211 | 54793 |
| 68946 | 50360517 | 50361643 | 54793 |
| 68947 | 50364838 | 50370052 | 54793 |
| 68948 | 50377559 | 50377974 | 54793 |
| 68949 | 50383374 | 50385129 | 54793 |
| 68950 | 50416564 | 50418685 | 101115 |
| 68951 | 50600935 | 50602103 | |
| 68952 | 50672751 | 50673533 | 20256 |
| 68953 | 50807347 | 50809472 | 20256 |
| 68954 | 50847633 | 50848655 | 234948 |
| 68955 | 51442814 | 51443891 | |
| 68956 | 51468988 | 51469729 | |
| 68957 | 51475254 | 51476675 | 232286 |
| 68958 | 51539513 | 51540101 | |
| 68959 | 51541117 | 51541729 | |
| 68960 | 51558789 | 51564498 | |
| 68961 | 51581354 | 51583058 | |
| 68962 | 51587984 | 51592807 | 182463 |
| 68963 | 51608288 | 51609625 | 182463 |
| 68964 | 51693493 | 51700450 | 182463 |
| 68965 | 51703397 | 51704400 | 182463 |
| 68966 | 51778177 | 51781129 | 182463 |
| 68967 | 51833048 | 51834188 | 182463 |
| 68968 | 52010833 | 52011579 | 271774, 182463 |
| 68969 | 52195075 | 52197117 | 171940 |
| 68970 | 52198871 | 52199915 | 171940 |
| 68971 | 52208887 | 52210285 | 171940 |
| 68972 | 52210413 | 52210916 | 171940 |
| 68973 | 52210919 | 52212012 | 171940 |
| 68974 | 52237474 | 52241268 | 235415 |
| 68975 | 52258610 | 52260147 | |
| 68976 | 52268427 | 52270059 | |
| 68977 | 52293543 | 52296139 | |
| 68978 | 52315697 | 52316692 | |
| 68979 | 52327805 | 52329493 | |
| 68980 | 52353216 | 52357727 | |
| 68981 | 52379764 | 52381482 | |
| 68982 | 52389609 | 52392086 | |
| 68983 | 52417337 | 52417965 | 225563 |
| 68984 | 52421758 | 52422678 | |
| 68985 | 52436325 | 52437340 | |
| 68986 | 52445123 | 52447482 | |
| 68987 | 52482085 | 52484323 | |
| 68988 | 52494953 | 52495704 | |
| 68989 | 52530573 | 52533989 | |
| 68990 | 52542333 | 52543543 | |
| 68991 | 52683910 | 52684824 | 64787 |
| 68992 | 52686628 | 52687464 | 64787 |
| 68993 | 52701296 | 52701945 | |
| 68994 | 52719572 | 52721233 | |
| 68995 | 52723674 | 52725160 | |
| 68996 | 52731424 | 52733556 | |
| 68997 | 52738949 | 52741189 | |
| 68998 | 52748120 | 52751798 | |
| 68999 | 52757262 | 52757926 | |
| 69000 | 52824049 | 52825737 | 101132 |
| 69001 | 52837851 | 52838319 | 101132 |
| 69002 | 52961731 | 52962255 | |
| 69003 | 52980853 | 52981902 | |
| 69004 | 54087839 | 54088289 | |
| 69005 | 54252925 | 54253622 | |
| 69006 | 54254093 | 54254772 | |
| 69007 | 54457346 | 54458473 | |
| 69008 | 54576349 | 54577295 | 54803 |
| 69009 | 54933636 | 54935672 | 124098 |
| 69010 | 54966878 | 54968621 | 101138, 087586 |
| 69011 | 54986773 | 54987642 | 87589 |
| 69012 | 55043304 | 55044452 | 22277 |
| 69013 | 55129885 | 55130411 | |
| 69014 | 55362143 | 55364629 | |
| 69015 | 55373639 | 55374886 | |
| 69016 | 55705216 | 55705783 | |
| 69017 | 55925996 | 55927110 | 101146 |
| 69018 | 55963929 | 55965407 | 218018 |
| 69019 | 55965929 | 55967514 | 132819, 218018 |
| 69020 | 56003228 | 56004738 | |
| 69021 | 56010578 | 56011472 | |
| 69022 | 56014488 | 56014954 | |
| 69023 | 56015397 | 56017278 | |
| 69024 | 56017776 | 56018384 | |
| 69025 | 56031889 | 56032737 | |
| 69026 | 56069938 | 56070707 | |
| 69027 | 56127334 | 56128131 | |
| 69028 | 56130377 | 56131388 | |
| 69029 | 56133099 | 56135148 | |
| 69030 | 56135401 | 56136610 | 124253 |
| 69031 | 56161185 | 56161950 | |
| 69032 | 56164718 | 56165483 | |
| 69033 | 56323398 | 56324049 | |
| 69034 | 56472173 | 56473087 | |
| 69035 | 56826607 | 56827986 | 124224 |
| 69036 | 56881744 | 56882627 | 124224 |
| 69037 | 56884093 | 56886520 | 124209, 124224 |
| 69038 | 56949070 | 56949877 | |
| 69039 | 56963974 | 56965419 | 124164 |
| 69040 | 57007271 | 57008153 | 124164 |
| 69041 | 57011815 | 57012947 | 124164 |
| 69042 | 57224423 | 57227787 | 254995, 124222 |
| 69043 | 57267065 | 57269007 | 254995, 215440 |
| 69044 | 57463402 | 57464186 | 225806, 087460 |
| 69045 | 57466709 | 57468480 | 87460 |
| 69046 | 57555855 | 57557327 | 101158 |
| 69047 | 57570044 | 57572032 | 101160, 101158 |
| 69048 | 57579373 | 57582963 | 101160 |
| 69049 | 57606247 | 57608277 | 101166, 124172 |
| 69050 | 57616710 | 57618536 | 101166 |
| 69051 | 58515220 | 58516223 | 132825, 196227 |
| 69052 | 58839358 | 58839876 | 228340 |
| 69053 | 60577926 | 60578530 | 130699 |
| 69054 | 60602094 | 60602826 | 130699 |
| 69055 | 60638940 | 60639887 | 265306, 130699 |
| 69056 | 60640502 | 60642490 | 130699 |
| 69057 | 60663484 | 60663948 | |
| 69058 | 60696164 | 60698648 | 149657 |
| 69059 | 60709734 | 60711324 | 149657 |
| 69060 | 60716515 | 60719758 | 184402, 101182 |
| 69061 | 60721146 | 60721939 | 184402 |
| 69062 | 60752316 | 60752967 | 184402 |
| 69063 | 60753493 | 60755253 | 184402 |
| 69064 | 60757037 | 60759244 | 101181, 184402 |
| 69065 | 60762248 | 60763235 | 101181 |
| 69066 | 60812985 | 60814853 | 130703 |
| 69067 | 60871820 | 60873498 | |
| 69068 | 60876827 | 60879499 | 130706 |
| 69069 | 60924909 | 60925996 | 130702 |
| 69070 | 60927098 | 60931827 | 228812, 130702 |
| 69071 | 60931979 | 60932691 | 130702 |
| 69072 | 60933049 | 60933807 | 130702 |
| 69073 | 60939219 | 60940456 | 130702 |
| 69074 | 60941047 | 60946576 | 130702 |
| 69075 | 60951368 | 60952007 | |
| 69076 | 60952386 | 60953711 | |
| 69077 | 60960927 | 60963363 | 171858 |
| 69078 | 60981504 | 60983000 | 149679 |
| 69079 | 61282635 | 61284797 | 101187 |
| 69080 | 61404144 | 61405218 | |
| 69081 | 61407471 | 61408800 | |
| 69082 | 61426421 | 61427333 | |
| 69083 | 61427528 | 61430088 | 101189 |
| 69084 | 61435723 | 61438045 | 060491, 229873 |
| 69085 | 61462618 | 61463220 | 92758 |
| 69086 | 61553879 | 61558757 | 101191 |
| 69087 | 61560249 | 61560910 | 101191 |
| 69088 | 61568223 | 61570356 | 101193, 101191 |
| 69089 | 61583481 | 61585420 | 101194 |
| 69090 | 61586802 | 61587793 | 101194 |
| 69091 | 61733345 | 61734395 | 225978, 231133 |
| 69092 | 61846194 | 61848424 | 149658 |
| 69093 | 61903356 | 61905592 | 101199, 101198 |
| 69094 | 62151862 | 62152983 | 125534 |
| 69095 | 62168495 | 62170223 | 101213 |
| 69096 | 62204429 | 62205899 | 130589 |
| 69097 | 62257060 | 62260425 | 232442, 101216 |
| 69098 | 62289058 | 62289996 | 258366 |
| 69099 | 62337806 | 62340428 | 273154, 197114, 101246 |
| 69100 | 62359739 | 62360371 | 273154, 197114 |
| 69101 | 62365541 | 62366280 | 273154, 197114 |
| 69102 | 62366551 | 62367390 | 203896, 273154, 197114 |
| 69103 | 62368623 | 62369957 | 273047, 273154, 203896 |
| 69104 | 62370350 | 62371331 | 125520, 273154, 203896, |
| | | | 273047 |
| 69105 | 62371375 | 62373204 | 273047, 125520 |
| 69106 | 62494596 | 62497533 | 101150 |
| 69107 | 62526004 | 62528534 | 101152 |
| 69108 | 62586617 | 62589205 | 196700, 198276 |
| 69109 | 62599869 | 62601671 | 196700 |
| 69110 | 62609006 | 62609572 | 130590, 196700 |
| 69111 | 62609626 | 62611183 | 130590, 196700 |
| 69112 | 62611186 | 62613465 | 101161, 130590, 196700 |
| 69113 | 62669770 | 62670992 | 196421, 196700 |
| 69114 | 62693583 | 62698761 | 171703 |
| 69115 | 62710426 | 62711463 | 171700 |
| 69116 | 62853270 | 62854340 | 196132 |
| 69117 | 62867602 | 62868571 | 196132 |
| 69118 | 62869431 | 62870179 | 196132 |
| 69119 | 62873862 | 62874596 | |
| 69120 | 62886236 | 62888064 | 203880 |

The locations of ODSCs on Chromosome 21 are shown in Table 63.

TABLE 63
ODSC Locations on Chromosome 21

| SEQID NO. | Start | End | Overlapping Gene(s) |
|---|---|---|---|
| 69121 | 9825324 | 9827688 | 264063, 264462 |
| 69122 | 15754711 | 15756574 | 155304 |
| 69123 | 15964064 | 15964711 | 243440, 223662 |
| 69124 | 16072029 | 16072863 | |
| 69125 | 16110187 | 16110697 | |
| 69126 | 16235349 | 16238004 | 235609 |
| 69127 | 16248428 | 16253364 | 235609 |
| 69128 | 16259872 | 16262269 | |
| 69129 | 16430681 | 16432444 | 180530 |
| 69130 | 16433093 | 16439400 | 236471, 180530 |
| 69131 | 16493495 | 16495153 | |
| 69132 | 16512086 | 16514434 | |
| 69133 | 16536747 | 16537636 | |
| 69134 | 16567880 | 16568629 | |
| 69135 | 16582447 | 16584383 | |
| 69136 | 16594000 | 16597326 | |
| 69137 | 16696775 | 16697370 | |
| 69138 | 16788051 | 16788784 | |
| 69139 | 16813169 | 16814332 | |
| 69140 | 16814912 | 16818109 | |
| 69141 | 16827780 | 16828946 | |
| 69142 | 16886577 | 16887285 | |
| 69143 | 17031585 | 17032596 | |
| 69144 | 17039118 | 17039834 | |
| 69145 | 17101757 | 17103879 | 155313 |
| 69146 | 17113885 | 17114777 | 155313 |
| 69147 | 17133763 | 17134364 | 155313 |
| 69148 | 17452099 | 17453219 | 215386 |
| 69149 | 17565949 | 17569752 | 215386 |
| 69150 | 17583967 | 17584909 | 215386 |
| 69151 | 17590991 | 17591930 | 215386 |
| 69152 | 17652548 | 17653014 | 215386 |
| 69153 | 17674497 | 17676041 | 215386, 270071 |
| 69154 | 17780098 | 17780774 | 215386 |
| 69155 | 17791019 | 17797063 | 270139, 215386 |
| 69156 | 17894413 | 17895431 | 215386 |
| 69157 | 17904185 | 17910389 | 215386 |
| 69158 | 17935416 | 17937179 | 215386 |
| 69159 | 17941010 | 17941416 | 215386 |
| 69160 | 17958146 | 17961569 | 215386 |
| 69161 | 18012247 | 18013361 | 228798 |
| 69162 | 18016716 | 18017385 | 270093 |
| 69163 | 18791150 | 18792030 | |
| 69164 | 18823302 | 18824884 | |
| 69165 | 18833196 | 18836675 | |
| 69166 | 18839170 | 18840833 | |
| 69167 | 18844155 | 18845613 | |
| 69168 | 18847886 | 18848848 | |
| 69169 | 18869583 | 18870030 | |
| 69170 | 18882775 | 18887060 | 154639 |
| 69171 | 18895108 | 18895892 | 154639 |
| 69172 | 18914517 | 18915248 | 154639 |
| 69173 | 18982965 | 18985555 | 154640 |
| 69174 | 19165903 | 19166569 | 244676, 154642 |
| 69175 | 19190431 | 19192513 | 154642 |
| 69176 | 22658638 | 22659163 | 154654 |
| 69177 | 23433247 | 23436953 | 233215 |
| 69178 | 23683945 | 23684579 | 226043 |
| 69179 | 26374707 | 26375604 | 226983 |
| 69180 | 26829143 | 26830282 | |
| 69181 | 26978906 | 26981146 | 154719 |
| 69182 | 27105679 | 27109092 | 154727, 154723 |
| 69183 | 27251432 | 27252048 | |
| 69184 | 27288428 | 27289008 | 142192 |
| 69185 | 27298288 | 27298634 | 142192 |
| 69186 | 27306626 | 27307759 | 142192, 233783 |
| 69187 | 27316289 | 27316932 | 142192 |
| 69188 | 27321755 | 27322743 | 142192 |
| 69189 | 27440950 | 27442079 | 142192 |
| 69190 | 27496663 | 27497137 | 142192 |
| 69191 | 27497147 | 27497641 | 142192 |
| 69192 | 27512345 | 27513028 | 142192 |
| 69193 | 27532239 | 27532845 | 142192, 224541 |
| 69194 | 27541149 | 27544105 | 273492, 142192, 224541 |
| 69195 | 28214829 | 28216879 | 154734 |
| 69196 | 28217423 | 28218041 | 154734 |
| 69197 | 28292120 | 28293258 | 154736, 223563 |
| 69198 | 29627650 | 29630179 | |
| 69199 | 29747077 | 29749603 | |
| 69200 | 30143864 | 30145856 | |
| 69201 | 30237808 | 30238479 | |
| 69202 | 30256725 | 30258000 | 156239 |
| 69203 | 30331722 | 30332176 | 198862 |
| 69204 | 30364238 | 30365724 | 198862 |
| 69205 | 30374313 | 30375975 | |
| 69206 | 30390734 | 30392115 | 156253 |
| 69207 | 30396183 | 30398304 | 273254, 156256 |
| 69208 | 30444840 | 30446889 | 156261 |
| 69209 | 30462229 | 30465889 | 156265 |
| 69210 | 30544301 | 30545349 | 156265 |
| 69211 | 30546444 | 30548360 | 156265 |
| 69212 | 30553627 | 30556089 | 224649 |
| 69213 | 30556162 | 30561012 | 224649 |
| 69214 | 30562480 | 30564444 | |
| 69215 | 30566282 | 30569877 | 156273, 215533 |
| 69216 | 30631058 | 30631632 | 156273, 215533 |
| 69217 | 30670278 | 30673016 | 156273 |
| 69218 | 30719292 | 30720285 | 156273 |
| 69219 | 30906548 | 30907155 | 156273 |
| 69220 | 31097185 | 31097921 | 171189 |
| 69221 | 31159463 | 31160300 | 171189 |
| 69222 | 32671535 | 32672401 | 156299 |
| 69223 | 32726069 | 32728145 | 156299 |
| 69224 | 32788795 | 32789303 | 156299 |
| 69225 | 32997009 | 32997976 | |
| 69226 | 33030057 | 33033315 | 142168, 234509 |
| 69227 | 33082802 | 33083480 | 156304 |
| 69228 | 33102844 | 33105691 | 156304 |
| 69229 | 33272318 | 33273455 | 142149 |
| 69230 | 33355280 | 33356286 | 142149 |
| 69231 | 33651087 | 33652503 | 159055, 227256 |
| 69232 | 33670528 | 33671115 | 170262 |
| 69233 | 33758427 | 33759000 | 142207 |
| 69234 | 33764490 | 33766333 | 256073, 142207 |
| 69235 | 33783865 | 33785067 | 166979 |
| 69236 | 33965179 | 33966238 | 265590, 159079 |
| 69237 | 33983871 | 33985948 | 265590, 159079 |
| 69238 | 34099097 | 34101275 | 238197, 159082 |
| 69239 | 34142959 | 34145141 | 205930, 159086 |
| 69240 | 34383203 | 34384209 | 227757 |
| 69241 | 34569003 | 34570261 | |
| 69242 | 34573973 | 34577771 | |
| 69243 | 34579490 | 34580592 | |
| 69244 | 34587249 | 34589087 | |
| 69245 | 34597542 | 34600443 | |
| 69246 | 34601866 | 34604152 | 159110 |
| 69247 | 34638348 | 34639842 | 243646, 223799, 249624 |
| 69248 | 34658321 | 34659860 | 243646 |
| 69249 | 34696319 | 34698073 | 142166 |
| 69250 | 34733025 | 34734180 | |
| 69251 | 34752042 | 34753449 | |
| 69252 | 34754368 | 34756390 | |
| 69253 | 34774938 | 34778418 | 159128 |
| 69254 | 34785116 | 34785931 | 159128 |
| 69255 | 34806933 | 34807438 | 142188, 159128 |
| 69256 | 34823828 | 34825117 | 142188, 159128 |
| 69257 | 34851138 | 34853235 | 142188, 159128 |
| 69258 | 34863419 | 34864629 | 177692 |
| 69259 | 34913581 | 34917383 | 159140, 159131 |
| 69260 | 34959935 | 34962117 | 205758, 159147, 249209 |
| 69261 | 35013486 | 35017651 | 205726, 205758, 249209 |
| 69262 | 35027827 | 35028603 | 205726, 249209 |
| 69263 | 35040679 | 35041399 | 205726, 249209 |
| 69264 | 35103747 | 35104525 | 205726, 249209 |
| 69265 | 35133147 | 35136232 | 205726, 249209 |
| 69266 | 35155112 | 35156738 | 205726, 249209 |
| 69267 | 35171506 | 35172402 | 205726, 249209 |
| 69268 | 35173873 | 35174661 | 205726, 249209 |
| 69269 | 35287362 | 35288563 | 237945, 241837 |
| 69270 | 35319735 | 35320843 | 237945 |
| 69271 | 35340192 | 35342337 | 273102, 237945 |
| 69272 | 35352505 | 35353128 | |
| 69273 | 35412053 | 35412796 | |
| 69274 | 35444787 | 35447164 | 198743, 272962, 272657, |
| | | | 243927 |
| 69275 | 35467207 | 35467577 | 198743, 272962, 272657, |
| | | | 243927 |
| 69276 | 35528884 | 35531215 | 272657 |
| 69277 | 35574049 | 35575400 | 272657 |
| 69278 | 35733929 | 35734805 | |
| 69279 | 35746697 | 35748869 | 205670, 225555 |
| 69280 | 35883661 | 35884878 | 180509 |
| 69281 | 35892449 | 35901456 | 159200 |
| 69282 | 35908284 | 35910284 | 159200 |
| 69283 | 35915292 | 35916719 | 159200 |
| 69284 | 35916741 | 35918719 | 159200 |
| 69285 | 35944243 | 35944825 | 159200 |
| 69286 | 35948236 | 35948666 | 159200 |
| 69287 | 35986113 | 35988278 | 159200 |
| 69288 | 36100511 | 36101197 | 230978 |
| 69289 | 36109279 | 36110160 | 230978 |
| 69290 | 36166350 | 36175575 | 159216 |
| 69291 | 36178900 | 36182125 | 159216 |
| 69292 | 36184112 | 36186145 | 159216 |
| 69293 | 36189619 | 36191737 | 159216 |
| 69294 | 36205431 | 36212400 | 159216 |
| 69295 | 36215827 | 36217426 | 159216 |
| 69296 | 36237200 | 36238133 | 159216 |
| 69297 | 36249605 | 36250287 | 159216 |
| 69298 | 36253407 | 36254952 | 159216 |
| 69299 | 36257048 | 36263244 | 159216 |
| 69300 | 36280531 | 36281299 | 159216 |
| 69301 | 36282366 | 36283105 | 159216 |
| 69302 | 36298871 | 36299816 | 159216 |
| 69303 | 36423375 | 36424144 | 159216 |
| 69304 | 36435523 | 36436518 | 159216 |
| 69305 | 36508897 | 36509499 | 234703, 159216 |
| 69306 | 36569006 | 36571019 | 159216 |
| 69307 | 36598478 | 36599138 | 159216 |
| 69308 | 36628625 | 36630030 | 159216 |
| 69309 | 36674780 | 36675954 | 159216 |
| 69310 | 37049351 | 37049971 | 159216 |
| 69311 | 37431753 | 37433760 | 236677, 185917 |
| 69312 | 37441081 | 37443173 | 230212, 159228, 185917 |
| 69313 | 37445037 | 37446028 | 159228, 185917, 230212 |
| 69314 | 37452087 | 37453218 | 230212 |
| 69315 | 37459694 | 37460098 | 236119, 230212 |
| 69316 | 37484536 | 37486762 | 230212 |
| 69317 | 37499405 | 37500018 | |
| 69318 | 37506419 | 37507871 | 159231, 236830 |
| 69319 | 37507874 | 37508460 | 159231, 236830 |
| 69320 | 37513629 | 37514428 | 159231, 236830 |
| 69321 | 37528726 | 37530180 | 142197 |
| 69322 | 37560285 | 37561127 | 142197 |
| 69323 | 37573697 | 37577091 | 142197 |
| 69324 | 37581645 | 37582772 | 142197 |
| 69325 | 37607392 | 37608178 | 142197 |
| 69326 | 37632480 | 37633302 | 142197 |
| 69327 | 37691636 | 37694871 | 273199, 159256 |
| 69328 | 37697469 | 37698292 | 159256 |
| 69329 | 37808953 | 37810602 | 230479 |
| 69330 | 37833608 | 37835209 | 230479, 159261, 233818 |
| 69331 | 37843001 | 37845330 | 230479, 159261, 233818 |
| 69332 | 37848941 | 37854683 | 230479, 159261, 233818 |
| 69333 | 37871658 | 37874466 | 159261, 233818 |
| 69334 | 37918112 | 37918902 | 159261 |
| 69335 | 38145363 | 38145878 | 159267 |
| 69336 | 38219231 | 38220106 | 159267 |
| 69337 | 38230466 | 38234581 | 159267 |
| 69338 | 38300809 | 38302349 | 159267 |
| 69339 | 38337893 | 38339974 | 224790, 159267 |
| 69340 | 38352441 | 38353554 | 159267 |
| 69341 | 38361583 | 38363376 | 159267 |
| 69342 | 38444049 | 38447094 | 182670, 185808 |
| 69343 | 38526095 | 38526822 | 182670 |
| 69344 | 38592703 | 38594063 | 242553, 230366 |
| 69345 | 38623283 | 38623694 | 157538 |
| 69346 | 38629344 | 38631115 | 157538 |
| 69347 | 38638611 | 38641102 | 272948, 157538 |
| 69348 | 38709976 | 38712340 | |
| 69349 | 38737269 | 38742245 | 273210, 157540 |
| 69350 | 38802600 | 38803146 | 157540 |
| 69351 | 38824178 | 38824924 | 157540 |
| 69352 | 38835289 | 38836766 | 157540 |
| 69353 | 38935244 | 38938676 | |
| 69354 | 38999253 | 39000176 | 157542 |
| 69355 | 39003622 | 39004640 | 157542 |
| 69356 | 39030482 | 39032735 | 157542 |
| 69357 | 39046057 | 39046795 | 157542 |
| 69358 | 39076646 | 39077631 | 157542 |
| 69359 | 39159706 | 39160370 | 157542 |
| 69360 | 39201333 | 39202439 | 157542 |
| 69361 | 39216808 | 39218737 | 157542 |
| 69362 | 39221885 | 39222647 | 157542 |
| 69363 | 39511265 | 39513106 | 198054 |
| 69364 | 39543597 | 39544329 | 198054, 157551 |
| 69365 | 39599146 | 39600467 | 157551 |
| 69366 | 39664211 | 39666377 | 157551 |
| 69367 | 39684669 | 39685280 | |
| 69368 | 39706258 | 39707698 | |
| 69369 | 39714554 | 39716196 | |
| 69370 | 39877533 | 39878072 | 157554 |
| 69371 | 40105706 | 40106991 | |
| 69372 | 40138639 | 40140351 | |
| 69373 | 40157997 | 40161004 | |
| 69374 | 40164563 | 40171478 | |
| 69375 | 40174778 | 40185779 | 157557 |
| 69376 | 40192159 | 40193947 | 157557 |
| 69377 | 40195567 | 40197103 | 157557 |
| 69378 | 40202585 | 40204982 | |
| 69379 | 40218859 | 40220647 | 229986 |
| 69380 | 40224209 | 40226679 | |
| 69381 | 40250265 | 40253692 | 205622 |
| 69382 | 40269228 | 40270927 | 229925, 205622 |
| 69383 | 40274524 | 40276993 | 229925, 205622 |
| 69384 | 40278256 | 40285915 | 234035, 205622 |
| 69385 | 40285919 | 40287320 | 234035, 205622 |
| 69386 | 40290099 | 40291464 | 205622 |
| 69387 | 40329586 | 40333063 | |
| 69388 | 40365379 | 40366307 | 235888 |
| 69389 | 40392317 | 40394371 | |
| 69390 | 40394485 | 40395775 | |
| 69391 | 40405316 | 40407107 | |
| 69392 | 40466104 | 40468329 | |
| 69393 | 40554296 | 40556470 | 185658, 272991, 183527 |
| 69394 | 40684340 | 40686809 | 255568, 185658 |
| 69395 | 40719856 | 40722209 | 205581 |
| 69396 | 40756558 | 40758442 | 182093 |
| 69397 | 40816857 | 40818436 | 185437, 157578 |
| 69398 | 40899568 | 40900205 | 235012 |
| 69399 | 41879370 | 41880673 | 171587 |
| 69400 | 42505484 | 42506288 | |
| 69401 | 42512702 | 42513608 | 226496 |
| 69402 | 42513650 | 42514606 | 226496 |
| 69403 | 42791495 | 42793234 | 157601 |
| 69404 | 42797328 | 42799158 | 157601 |
| 69405 | 42839173 | 42839769 | 184012 |
| 69406 | 42849511 | 42851098 | 184012 |
| 69407 | 42851757 | 42852483 | 184012 |
| 69408 | 42872413 | 42874945 | 184012 |
| 69409 | 42878612 | 42881197 | 184012 |
| 69410 | 42882160 | 42883016 | 184012 |
| 69411 | 42897751 | 42899100 | 184012 |
| 69412 | 42933412 | 42935263 | 232806 |
| 69413 | 42950608 | 42955693 | 223400 |
| 69414 | 43032042 | 43033614 | |
| 69415 | 43040967 | 43041545 | |
| 69416 | 43068381 | 43069523 | |
| 69417 | 43073733 | 43074839 | |
| 69418 | 43080265 | 43081181 | |
| 69419 | 43083766 | 43085497 | |
| 69420 | 43097696 | 43099739 | 227702 |
| 69421 | 43126619 | 43127271 | |
| 69422 | 43135300 | 43136579 | 236384 |
| 69423 | 43170351 | 43171066 | 183421 |
| 69424 | 43178088 | 43180142 | 183421 |
| 69425 | 43181253 | 43182881 | 183421 |
| 69426 | 43184740 | 43190254 | 183421 |
| 69427 | 43298368 | 43300479 | 141956 |
| 69428 | 43345652 | 43347235 | 157617 |
| 69429 | 43352193 | 43353352 | 157617 |
| 69430 | 43373182 | 43374752 | 157617 |
| 69431 | 43378116 | 43379126 | |
| 69432 | 43428714 | 43431664 | 237232, 173276 |
| 69433 | 43457927 | 43458528 | |
| 69434 | 43459388 | 43460168 | |
| 69435 | 43478699 | 43479346 | |
| 69436 | 43517885 | 43518322 | 177398 |
| 69437 | 43534249 | 43534618 | 177398 |
| 69438 | 43619098 | 43620434 | 160179 |
| 69439 | 43638996 | 43640948 | 160179 |
| 69440 | 43647944 | 43648766 | 160179 |
| 69441 | 43653060 | 43655738 | 160179 |
| 69442 | 43757856 | 43758457 | |
| 69443 | 43806162 | 43806765 | 160183 |
| 69444 | 43853541 | 43854797 | 160185 |
| 69445 | 43869829 | 43870517 | |
| 69446 | 43882306 | 43883165 | |
| 69447 | 43884943 | 43886004 | |
| 69448 | 43889091 | 43890194 | |
| 69449 | 43893729 | 43895039 | 160188 |
| 69450 | 43933304 | 43935412 | 160190 |
| 69451 | 43946140 | 43947098 | 160190 |
| 69452 | 43947238 | 43948527 | 160190 |
| 69453 | 43955732 | 43956269 | 160190 |
| 69454 | 43959199 | 43960146 | 160190 |
| 69455 | 44035004 | 44035498 | 225431 |
| 69456 | 44044281 | 44045073 | |
| 69457 | 44050651 | 44051127 | |
| 69458 | 44073663 | 44074866 | 160191 |
| 69459 | 44139749 | 44140420 | 160191 |
| 69460 | 44153103 | 44153802 | 225731, 160191 |
| 69461 | 44154186 | 44155144 | 225731, 160191 |
| 69462 | 44157876 | 44159005 | 225731, 160191 |
| 69463 | 44298318 | 44300476 | 160194, 160193 |
| 69464 | 44312305 | 44314546 | 160194 |
| 69465 | 44360233 | 44360582 | |
| 69466 | 44393510 | 44396542 | 160199 |
| 69467 | 44465322 | 44468171 | |
| 69468 | 44484650 | 44485334 | 160200 |
| 69469 | 44487600 | 44488901 | 160200 |
| 69470 | 44492582 | 44494001 | 160200 |
| 69471 | 44494067 | 44497179 | 160200 |
| 69472 | 44526651 | 44528907 | 160201 |
| 69473 | 44535770 | 44536747 | 232777 |
| 69474 | 44558950 | 44559525 | |
| 69475 | 44566730 | 44567255 | |
| 69476 | 44582312 | 44583456 | 228120 |
| 69477 | 44588765 | 44594053 | 160202 |
| 69478 | 44596302 | 44598028 | |
| 69479 | 44630569 | 44632539 | |
| 69480 | 44693518 | 44694777 | |
| 69481 | 44707697 | 44708822 | |
| 69482 | 44752945 | 44753919 | |
| 69483 | 44762616 | 44763811 | |
| 69484 | 44823718 | 44824562 | |
| 69485 | 44826956 | 44827495 | |
| 69486 | 44843643 | 44851961 | 142178 |
| 69487 | 44858767 | 44863168 | |
| 69488 | 44865493 | 44868180 | 188660 |
| 69489 | 44881761 | 44887051 | 185186, 223975 |
| 69490 | 44895890 | 44899879 | 185186 |
| 69491 | 44912946 | 44920570 | |
| 69492 | 44921141 | 44925353 | |
| 69493 | 44929288 | 44941389 | |
| 69494 | 45004342 | 45007530 | 160207 |
| 69495 | 45029319 | 45030097 | 160207 |
| 69496 | 45059580 | 45060579 | 160207 |
| 69497 | 45063909 | 45066990 | 160207 |
| 69498 | 45078407 | 45080431 | 160208, 160207 |
| 69499 | 45138576 | 45140618 | 160209 |
| 69500 | 45145608 | 45146189 | 160209 |
| 69501 | 45146791 | 45149662 | 160209 |
| 69502 | 45157821 | 45158484 | 160209 |
| 69503 | 45177445 | 45178834 | 160209 |
| 69504 | 45195033 | 45196965 | 160213 |
| 69505 | 45202413 | 45203795 | 234030 |
| 69506 | 45208455 | 45210419 | 160214 |
| 69507 | 45231906 | 45232714 | 215458 |
| 69508 | 45283970 | 45288772 | 160216 |
| 69509 | 45300083 | 45300571 | 160216 |
| 69510 | 45303913 | 45304386 | 160216 |
| 69511 | 45331037 | 45332377 | 160216 |
| 69512 | 45343282 | 45345748 | 160216 |
| 69513 | 45358319 | 45359490 | 160216 |
| 69514 | 45431011 | 45433421 | 160218 |
| 69515 | 45526976 | 45527952 | 241945 |
| 69516 | 45552691 | 45556228 | 160221 |
| 69517 | 45557810 | 45558404 | 160221 |
| 69518 | 45589964 | 45591732 | 267913 |
| 69519 | 45591995 | 45593064 | 267913 |
| 69520 | 45595854 | 45596473 | 237604 |
| 69521 | 45604291 | 45605754 | |
| 69522 | 45659973 | 45664367 | 160223 |
| 69523 | 45667744 | 45671267 | 232010, 142182 |
| 69524 | 45717402 | 45718107 | 160224 |
| 69525 | 45719252 | 45721094 | 141959 |
| 69526 | 45758710 | 45760064 | 232969, 160226 |
| 69527 | 45773100 | 45774261 | 142185 |
| 69528 | 45858441 | 45859044 | 264452, 142185 |
| 69529 | 45875152 | 45876855 | 160233, 229356 |
| 69530 | 45877453 | 45878165 | 160233 |
| 69531 | 45897736 | 45899164 | |
| 69532 | 45902231 | 45904779 | |
| 69533 | 45904960 | 45905523 | 228709 |
| 69534 | 45925524 | 45926012 | 175894 |
| 69535 | 45937429 | 45937898 | 182912, 175894 |
| 69536 | 46156382 | 46156965 | |
| 69537 | 46198292 | 46199067 | 184787 |
| 69538 | 46205307 | 46206256 | 184787 |
| 69539 | 46220781 | 46222792 | 236519, 184787 |
| 69540 | 46237484 | 46239024 | 184900 |
| 69541 | 46288656 | 46295866 | 183255 |
| 69542 | 46297751 | 46299040 | |
| 69543 | 46305518 | 46305975 | 160255 |
| 69544 | 46330948 | 46332706 | 160255 |
| 69545 | 46333264 | 46334627 | 160255 |
| 69546 | 46351855 | 46353110 | 183250, 160255 |
| 69547 | 46359015 | 46360926 | 160256, 183250 |
| 69548 | 46369485 | 46370152 | 160256 |
| 69549 | 46387862 | 46388597 | 160256 |
| 69550 | 46493787 | 46495049 | 235374, 197381 |
| 69551 | 46707157 | 46708885 | 215447, 186866 |
| 69552 | 46728290 | 46729415 | |
| 69553 | 46738578 | 46739212 | |
| 69554 | 46748609 | 46751694 | |
| 69555 | 46783388 | 46786435 | |
| 69556 | 46824648 | 46826304 | 182871 |
| 69557 | 46830210 | 46831016 | 182871 |
| 69558 | 46868376 | 46869382 | 182871 |
| 69559 | 46873962 | 46877003 | 182871 |
| 69560 | 46879128 | 46881629 | 182871 |
| 69561 | 46888503 | 46890405 | 182871 |
| 69562 | 46890424 | 46893425 | 182871 |
| 69563 | 46928424 | 46929269 | 173638, 182871 |
| 69564 | 46937076 | 46938105 | 173638 |
| 69565 | 46944679 | 46945959 | 173638 |
| 69566 | 46974433 | 46975110 | |
| 69567 | 47238395 | 47239585 | 183570 |
| 69568 | 47392859 | 47394658 | |
| 69569 | 47401034 | 47401519 | |
| 69570 | 47544166 | 47545990 | 142173 |
| 69571 | 47553601 | 47554798 | |
| 69572 | 47572746 | 47576960 | 160282 |
| 69573 | 47608261 | 47609384 | 228404, 160285 |
| 69574 | 47610483 | 47611669 | 160285 |
| 69575 | 47646039 | 47650050 | 215424, 160285 |
| 69576 | 47705160 | 47707220 | 182362, 160294 |
| 69577 | 47743872 | 47745565 | 160299 |
| 69578 | 47813429 | 47814745 | 160299 |
| 69579 | 47877415 | 47879991 | 160305 |
| 69580 | 47897613 | 47898153 | 160305 |
| 69581 | 48054115 | 48056171 | 160310 |

The locations of ODSCs on Chromosome 22 are shown in Table 64.

TABLE 64
ODSC Locations on Chromosome 22

| SEQ ID NO. | Start | End | Overlapping Gene(s) |
|---|---|---|---|
| 69582 | 17565348 | 17566817 | 177663 |
| 69583 | 17633053 | 17634282 | 069998 |
| 69584 | 17639004 | 17641393 | 185837, 069998 |
| 69585 | 17651865 | 17653561 | |
| 69586 | 17681992 | 17682915 | 093072 |
| 69587 | 17709383 | 17710437 | |
| 69588 | 17710763 | 17711825 | |
| 69589 | 17812572 | 17813306 | |
| 69590 | 17819813 | 17820701 | |
| 69591 | 17848960 | 17851877 | 099954 |
| 69592 | 17861348 | 17861850 | 099954 |
| 69593 | 17886691 | 17888886 | 099954 |
| 69594 | 18042317 | 18043423 | 182902 |
| 69595 | 18049226 | 18052057 | 182902 |
| 69596 | 18063085 | 18063851 | 236754, 182902 |
| 69597 | 18110473 | 18112719 | 099968, 131100 |
| 69598 | 18115422 | 18117313 | 099968 |
| 69599 | 18120204 | 18122809 | 099968 |
| 69600 | 18188589 | 18190453 | 099968 |
| 69601 | 18242822 | 18244389 | 015475 |
| 69602 | 18251721 | 18253635 | 015475 |
| 69603 | 18256181 | 18257809 | 015475 |
| 69604 | 18259609 | 18261149 | 269220 |
| 69605 | 18263851 | 18265044 | |
| 69606 | 18269146 | 18270038 | |
| 69607 | 18319880 | 18320572 | 243156 |
| 69608 | 18320776 | 18321728 | 243156 |
| 69609 | 18323720 | 18325868 | 243156 |
| 69610 | 18334441 | 18334899 | 243156 |
| 69611 | 18339645 | 18340162 | 243156 |
| 69612 | 18372709 | 18374138 | 243156 |
| 69613 | 18405826 | 18406422 | 243156 |
| 69614 | 18421087 | 18421680 | 243156 |
| 69615 | 18454962 | 18456130 | 243156 |
| 69616 | 18483061 | 18484901 | 243156 |
| 69617 | 18495169 | 18495838 | 243156 |
| 69618 | 18505659 | 18508079 | 243156 |
| 69619 | 18518437 | 18519455 | 235295 |
| 69620 | 18520630 | 18521779 | 235295 |
| 69621 | 18525106 | 18525980 | |
| 69622 | 18526431 | 18528332 | |
| 69623 | 18538156 | 18542553 | |
| 69624 | 18542611 | 18546250 | |
| 69625 | 18549041 | 18552549 | |
| 69626 | 18558277 | 18562092 | 215193, 225335 |
| 69627 | 18567487 | 18568677 | 215193 |
| 69628 | 18573948 | 18575608 | 215193 |
| 69629 | 18592905 | 18593947 | 183785, 215193 |
| 69630 | 18632202 | 18633662 | 184979 |
| 69631 | 18892944 | 18894090 | 183628 |
| 69632 | 18957681 | 18958708 | 237517 |
| 69633 | 18967407 | 18967937 | 237517 |
| 69634 | 19057772 | 19058664 | 070413 |
| 69635 | 19096867 | 19097471 | 070413 |
| 69636 | 19107679 | 19110218 | 223461, 070413 |
| 69637 | 19131432 | 19132698 | 100056 |
| 69638 | 19158047 | 19160349 | 260924 |
| 69639 | 19161217 | 19163179 | 100075 |
| 69640 | 19164738 | 19167381 | 070371, 100075 |
| 69641 | 19258795 | 19260370 | 070371 |
| 69642 | 19278264 | 19280695 | 070371 |
| 69643 | 19323471 | 19324019 | 100084 |
| 69644 | 19417726 | 19421110 | 185608, 100084, 242259 |
| 69645 | 19434203 | 19436161 | 185065, 100084, 242259 |
| 69646 | 19465245 | 19467542 | 093009, 070010 |
| 69647 | 19531340 | 19532604 | |
| 69648 | 19555438 | 19557316 | |
| 69649 | 19585642 | 19586397 | |
| 69650 | 19591163 | 19596399 | |
| 69651 | 19811258 | 19812336 | 185838 |
| 69652 | 19828218 | 19829058 | 185838 |
| 69653 | 19835986 | 19836819 | 215012, 185838 |
| 69654 | 19841160 | 19844053 | 215012, 185838 |
| 69655 | 19928061 | 19931417 | 093010, 184470 |
| 69656 | 19940474 | 19941672 | 093010 |
| 69657 | 19946300 | 19951589 | 265087, 093010 |
| 69658 | 19957853 | 19959274 | 099889 |
| 69659 | 19970687 | 19972879 | 099889 |
| 69660 | 19973738 | 19974910 | 099889 |
| 69661 | 20007808 | 20010271 | 183597 |
| 69662 | 20067058 | 20069006 | 128191 |
| 69663 | 20080923 | 20081959 | 128191 |
| 69664 | 20103647 | 20106136 | 099899, 099901 |
| 69665 | 20116259 | 20117395 | 099904 |
| 69666 | 20118494 | 20120327 | 099904 |
| 69667 | 20190238 | 20192389 | 249923 |
| 69668 | 20198851 | 20199914 | |
| 69669 | 20209016 | 20210415 | |
| 69670 | 20223021 | 20223672 | |
| 69671 | 20286954 | 20287583 | 161132 |
| 69672 | 20307327 | 20308611 | 273139, 128185 |
| 69673 | 20747606 | 20748913 | 185252 |
| 69674 | 20848823 | 20851288 | 099917, 099910 |
| 69675 | 20857950 | 20859105 | 099917 |
| 69676 | 20861257 | 20864321 | 099917 |
| 69677 | 20878301 | 20878745 | 099917 |
| 69678 | 21127986 | 21128678 | 099937, 241973 |
| 69679 | 21130361 | 21131657 | 241973, 099937 |
| 69680 | 21131798 | 21137560 | 241973, 099937 |
| 69681 | 21144931 | 21145596 | 241973 |
| 69682 | 21161551 | 21162067 | 241973 |
| 69683 | 21212124 | 21214163 | 099940, 241973 |
| 69684 | 21221320 | 21222224 | 099940 |
| 69685 | 21234630 | 21236815 | 099940 |
| 69686 | 21237853 | 21238487 | 099940 |
| 69687 | 21239557 | 21240577 | 099940 |
| 69688 | 21258158 | 21259935 | |
| 69689 | 21270723 | 21272861 | 099942 |
| 69690 | 21292470 | 21293323 | 099942 |
| 69691 | 21305551 | 21307600 | 099942 |
| 69692 | 21307965 | 21308739 | 099942 |
| 69693 | 21311075 | 21312440 | 237476 |
| 69694 | 21335449 | 21337563 | 272829, 183773, 099949 |
| 69695 | 21355212 | 21357828 | 161149, 230513, 184436 |
| 69696 | 21811202 | 21812180 | |
| 69697 | 21921653 | 21923649 | 185651 |
| 69698 | 21983375 | 21984623 | 161179 |
| 69699 | 21995547 | 21997667 | 128228, 273342 |
| 69700 | 22019650 | 22021490 | 100023 |
| 69701 | 22072757 | 22073455 | 100027 |
| 69702 | 22089373 | 22090441 | 100027 |
| 69703 | 22191366 | 22192569 | 100030 |
| 69704 | 22220601 | 22222733 | 100030 |
| 69705 | 22291300 | 22293361 | 224086, 100034 |
| 69706 | 22306475 | 22307716 | 100034 |
| 69707 | 22315667 | 22316782 | 100038 |
| 69708 | 22336268 | 22337590 | 100038 |
| 69709 | 22344612 | 22345694 | 197549 |
| 69710 | 22428619 | 22430055 | 253242 |
| 69711 | 22440084 | 22441231 | 253823 |
| 69712 | 22442407 | 22443770 | |
| 69713 | 23033548 | 23038221 | 223999, 253822 |
| 69714 | 23043842 | 23048369 | 211661 |
| 69715 | 23094975 | 23096672 | |
| 69716 | 23302161 | 23303007 | |
| 69717 | 23521450 | 23523723 | 236794, 186716 |
| 69718 | 23546731 | 23547181 | 186716 |
| 69719 | 23547585 | 23548294 | 186716 |
| 69720 | 23558813 | 23559369 | 186716 |
| 69721 | 23593407 | 23594430 | 186716 |
| 69722 | 23598859 | 23599719 | 186716 |
| 69723 | 23606752 | 23607228 | 186716 |
| 69724 | 23632078 | 23633062 | 186716 |
| 69725 | 23699040 | 23699621 | |
| 69726 | 23863710 | 23864310 | |
| 69727 | 24042903 | 24043345 | 273000, 228315, 272578 |
| 69728 | 24058744 | 24059982 | 273000, 228315, 272578 |
| 69729 | 24093075 | 24094314 | 187792 |
| 69730 | 24107319 | 24108656 | 250479, 169314 |
| 69731 | 24108849 | 24111043 | 099953, 250479 |
| 69732 | 24114737 | 24115338 | 099953 |
| 69733 | 24128470 | 24130109 | 099956 |
| 69734 | 24191477 | 24192891 | |
| 69735 | 24235695 | 24237961 | 218537, 240972, 251357 |
| 69736 | 24255259 | 24256951 | |
| 69737 | 24407427 | 24408360 | 099991 |
| 69738 | 24439178 | 24439902 | 099991 |
| 69739 | 24463526 | 24464064 | 099991 |
| 69740 | 24551574 | 24552175 | 099991 |
| 69741 | 24552656 | 24553286 | 099991 |
| 69742 | 24602675 | 24605057 | |
| 69743 | 24666125 | 24668255 | 258555, 100014 |
| 69744 | 24684681 | 24686683 | 258555, 100014 |
| 69745 | 24687906 | 24688525 | 258555, 100014 |
| 69746 | 24699442 | 24700272 | 258555, 100014 |
| 69747 | 24718618 | 24720713 | 258555, 100014 |
| 69748 | 24797979 | 24799479 | 258555, 100014 |
| 69749 | 24877279 | 24879685 | 178803, 100024 |
| 69750 | 24882400 | 24882999 | 178803, 100024 |
| 69751 | 24889870 | 24892282 | 178803, 100024 |
| 69752 | 24895482 | 24897003 | 100024 |
| 69753 | 24898906 | 24900650 | 100024 |
| 69754 | 24901007 | 24901732 | 100024 |
| 69755 | 24902115 | 24903414 | 100024 |
| 69756 | 24906325 | 24907566 | 100024 |
| 69757 | 24909936 | 24910721 | 100024 |
| 69758 | 24912024 | 24912694 | 228923, 100024 |
| 69759 | 24920945 | 24921988 | 100024 |
| 69760 | 24931882 | 24932563 | |
| 69761 | 24938453 | 24940304 | 138867 |
| 69762 | 24940964 | 24941756 | 138867 |
| 69763 | 24944662 | 24948315 | 138867 |
| 69764 | 24948835 | 24953085 | 100028, 138867 |
| 69765 | 24960420 | 24960843 | 100028 |
| 69766 | 24967277 | 24968440 | 100028 |
| 69767 | 24972622 | 24973181 | 100028 |
| 69768 | 24974521 | 24975530 | 100028 |
| 69769 | 24976150 | 24976710 | 100028 |
| 69770 | 24988387 | 24990528 | 178026, 100028, 100031 |
| 69771 | 24997255 | 24998414 | 100028, 100031 |
| 69772 | 25215715 | 25216555 | 167037 |
| 69773 | 25289585 | 25291453 | 167037 |
| 69774 | 25347918 | 25350440 | 197077 |
| 69775 | 25361756 | 25362833 | 197077 |
| 69776 | 25363938 | 25364690 | 197077 |
| 69777 | 25365282 | 25366511 | 197077 |
| 69778 | 25369643 | 25370294 | 197077 |
| 69779 | 25385578 | 25386134 | 197077 |
| 69780 | 25387515 | 25388748 | 197077 |
| 69781 | 25506084 | 25510561 | 203280, 197077 |
| 69782 | 25515425 | 25517412 | 197077 |
| 69783 | 25526476 | 25528123 | 197077 |
| 69784 | 25532491 | 25534127 | 197077 |
| 69785 | 25544452 | 25544842 | 197077 |
| 69786 | 25560924 | 25566875 | 197077 |
| 69787 | 25578597 | 25579509 | 197077 |
| 69788 | 25579810 | 25580511 | 197077 |
| 69789 | 25791716 | 25792416 | 100068 |
| 69790 | 25796799 | 25797389 | 100068 |
| 69791 | 25798060 | 25802096 | 100068 |
| 69792 | 25843647 | 25845061 | 100058 |
| 69793 | 26059289 | 26060390 | 100077 |
| 69794 | 26061840 | 26063480 | 100077 |
| 69795 | 26063868 | 26064855 | 100077 |
| 69796 | 26143069 | 26145195 | 133454 |
| 69797 | 26155633 | 26156314 | 133454 |
| 69798 | 26752585 | 26753019 | 100095 |
| 69799 | 26878784 | 26880676 | 100104, 100099 |
| 69800 | 26907448 | 26909387 | 261188, 100109 |
| 69801 | 26913337 | 26915078 | |
| 69802 | 26923533 | 26925937 | 128294 |
| 69803 | 26945794 | 26947928 | 128294 |
| 69804 | 26952708 | 26953471 | 128294 |
| 69805 | 26957568 | 26958242 | 128294 |
| 69806 | 26984990 | 26986864 | 128294 |
| 69807 | 27068343 | 27069865 | 244625, 206028, 225783 |
| 69808 | 27283240 | 27283999 | 231369 |
| 69809 | 27290352 | 27291772 | |
| 69810 | 27440605 | 27442500 | |
| 69811 | 27502475 | 27510640 | |
| 69812 | 27510768 | 27514108 | |
| 69813 | 27525287 | 27525632 | |
| 69814 | 27529734 | 27532301 | |
| 69815 | 27801695 | 27802558 | |
| 69816 | 27870854 | 27873787 | |
| 69817 | 27883932 | 27886530 | |
| 69818 | 27930404 | 27931058 | |
| 69819 | 28043249 | 28044144 | |
| 69820 | 28197209 | 28198984 | 169184 |
| 69821 | 28206588 | 28207079 | |
| 69822 | 28254572 | 28256147 | 180957 |
| 69823 | 28261959 | 28263416 | 180957 |
| 69824 | 28265959 | 28267456 | 180957 |
| 69825 | 28282086 | 28283029 | 180957 |
| 69826 | 28288697 | 28289234 | 180957 |
| 69827 | 28314325 | 28316448 | 235954, 180957 |
| 69828 | 28327486 | 28330228 | 235954 |
| 69829 | 28345278 | 28345974 | 235954 |
| 69830 | 28357966 | 28359079 | 235954 |
| 69831 | 28386782 | 28387437 | 100154, 235954 |
| 69832 | 28791712 | 28792817 | 100154 |
| 69833 | 28831362 | 28832252 | 100154 |
| 69834 | 28837662 | 28839880 | 100154 |
| 69835 | 29075213 | 29075744 | 100154 |
| 69836 | 29107717 | 29108839 | 183765 |
| 69837 | 29137557 | 29138921 | 100209, 183765 |
| 69838 | 29143161 | 29143867 | 100209 |
| 69839 | 29168195 | 29170078 | 159873 |
| 69840 | 29194498 | 29199333 | 226471, 100219 |
| 69841 | 29207658 | 29209157 | 226471 |
| 69842 | 29209201 | 29210380 | 226471 |
| 69843 | 29213806 | 29216280 | 226471 |
| 69844 | 29217095 | 29218191 | 226471 |
| 69845 | 29219052 | 29219515 | 226471 |
| 69846 | 29225072 | 29226124 | 226471 |
| 69847 | 29260805 | 29261302 | |
| 69848 | 29278758 | 29280710 | 183579 |
| 69849 | 29307532 | 29308535 | 183579 |
| 69850 | 29319642 | 29320426 | 183579 |
| 69851 | 29321112 | 29321997 | 183579 |
| 69852 | 29327663 | 29328559 | 183579 |
| 69853 | 29372649 | 29375856 | 183579 |
| 69854 | 29377484 | 29378628 | 183579 |
| 69855 | 29395675 | 29396692 | 183579, 235786 |
| 69856 | 29417754 | 29418588 | 183579 |
| 69857 | 29418987 | 29419697 | 183579 |
| 69858 | 29425779 | 29429279 | 177993, 183579 |
| 69859 | 29440106 | 29441228 | 183579 |
| 69860 | 29468419 | 29470419 | 183762 |
| 69861 | 29477752 | 29478394 | 183762 |
| 69862 | 29524619 | 29526579 | 183762 |
| 69863 | 29612631 | 29613755 | 186998 |
| 69864 | 29627920 | 29629086 | 186998 |
| 69865 | 29641358 | 29642575 | 186998 |
| 69866 | 29662403 | 29666189 | 182944, 100263 |
| 69867 | 29701956 | 29703896 | 185340 |
| 69868 | 29732730 | 29733507 | 100280 |
| 69869 | 29740118 | 29740499 | 100280 |
| 69870 | 29778546 | 29778971 | 100280 |
| 69871 | 29781154 | 29782134 | 100280 |
| 69872 | 29783416 | 29784867 | 100280 |
| 69873 | 29788790 | 29789438 | 100280 |
| 69874 | 29790646 | 29791425 | 100280 |
| 69875 | 29895758 | 29896354 | |
| 69876 | 29949157 | 29950717 | 100296 |
| 69877 | 29970787 | 29971806 | 184117 |
| 69878 | 29975688 | 29977419 | 184117 |
| 69879 | 29983845 | 29984510 | |
| 69880 | 29999008 | 30000493 | 186575 |
| 69881 | 30016582 | 30020911 | 186575 |
| 69882 | 30162141 | 30164392 | 184076, 100319 |
| 69883 | 30174732 | 30175447 | |
| 69884 | 30176849 | 30178921 | |
| 69885 | 30191565 | 30192127 | 100325 |
| 69886 | 30194004 | 30196175 | 100325 |
| 69887 | 30207206 | 30207873 | 100325 |
| 69888 | 30208961 | 30210434 | 100325 |
| 69889 | 30211511 | 30212537 | 100325 |
| 69890 | 30216955 | 30217853 | 100325 |
| 69891 | 30233260 | 30235086 | 100325 |
| 69892 | 30237923 | 30239217 | |
| 69893 | 30278885 | 30280146 | 100330 |
| 69894 | 30303702 | 30304111 | 100330 |
| 69895 | 30309992 | 30311218 | 100330 |
| 69896 | 30313176 | 30313847 | 100330 |
| 69897 | 30432611 | 30433199 | 227117 |
| 69898 | 30433779 | 30435261 | 227117 |
| 69899 | 30439472 | 30441358 | 227117 |
| 69900 | 30475693 | 30477449 | 176635, 227117 |
| 69901 | 30482935 | 30484058 | 176635 |
| 69902 | 30555328 | 30556486 | 176635 |
| 69903 | 30583438 | 30585137 | 225676 |
| 69904 | 30585854 | 30588390 | 225676 |
| 69905 | 30591309 | 30593204 | 225676 |
| 69906 | 30597858 | 30598385 | 225676 |
| 69907 | 30600338 | 30603907 | 225676 |
| 69908 | 30608164 | 30609797 | |
| 69909 | 30641056 | 30645114 | 268812, 128342 |
| 69910 | 30646145 | 30647613 | |
| 69911 | 30651934 | 30652386 | |
| 69912 | 30684804 | 30685703 | 239282, 248751 |
| 69913 | 30705616 | 30710652 | 099992 |
| 69914 | 30710851 | 30711864 | 099992 |
| 69915 | 30712860 | 30714354 | 099992 |
| 69916 | 30722135 | 30723138 | 099992 |
| 69917 | 30751939 | 30753191 | 187860, 099995 |
| 69918 | 30782374 | 30783856 | 099999 |
| 69919 | 30786941 | 30788194 | 099999 |
| 69920 | 30788376 | 30789762 | 099999 |
| 69921 | 30792486 | 30795547 | 100003, 099999 |
| 69922 | 30800013 | 30800808 | 100003, 099999 |
| 69923 | 30804101 | 30805999 | 249590, 100003, 099999 |
| 69924 | 30811662 | 30813601 | 100003, 249590, 099999 |
| 69925 | 30814259 | 30815332 | 268538, 100003, 249590, |
| | | | 099999 |
| 69926 | 30816061 | 30822970 | 273350, 222915, 242114, |
| | | | 272689, 100003, 099999, |
| | | | 249590 |
| 69927 | 30840597 | 30841951 | |
| 69928 | 30853877 | 30854426 | 100012 |
| 69929 | 30882471 | 30883178 | 181123 |
| 69930 | 30893361 | 30893984 | 133488 |
| 69931 | 30901032 | 30902153 | 133488 |
| 69932 | 30986943 | 30988505 | 100029 |
| 69933 | 31002275 | 31003796 | 185339, 100029, 223831 |
| 69934 | 31030291 | 31031095 | |
| 69935 | 31062945 | 31064855 | 167065, 100036 |
| 69936 | 31089995 | 31090619 | 184792 |
| 69937 | 31317522 | 31318485 | 235989 |
| 69938 | 31364085 | 31366479 | 133422 |
| 69939 | 31450842 | 31452289 | |
| 69940 | 31480081 | 31481666 | 183963 |
| 69941 | 31518515 | 31519142 | 185133 |
| 69942 | 31555378 | 31557070 | 264141, 138942 |
| 69943 | 31568761 | 31570068 | 138942 |
| 69944 | 31572719 | 31573949 | 138942 |
| 69945 | 31607477 | 31609274 | 182541 |
| 69946 | 31615828 | 31616632 | 182541 |
| 69947 | 31617682 | 31618163 | 182541 |
| 69948 | 31625500 | 31627645 | 202019, 182541 |
| 69949 | 31636855 | 31638705 | 182541 |
| 69950 | 31639894 | 31640413 | 182541 |
| 69951 | 31649553 | 31650626 | 182541 |
| 69952 | 31686131 | 31688688 | 228839, 100100 |
| 69953 | 31691678 | 31692632 | 228839 |
| 69954 | 31696712 | 31697348 | 228839 |
| 69955 | 31709235 | 31709776 | 228839 |
| 69956 | 31741590 | 31744076 | 213888, 100105 |
| 69957 | 31793552 | 31796671 | 185721 |
| 69958 | 31884929 | 31887156 | 184708, 185721, 198089 |
| 69959 | 31891896 | 31893267 | 184708, 185721, 198089 |
| 69960 | 31984996 | 31985729 | 198089 |
| 69961 | 32025855 | 32027446 | 241878 |
| 69962 | 32041769 | 32043799 | 241878 |
| 69963 | 32045734 | 32047125 | 241878 |
| 69964 | 32049060 | 32049814 | 241878 |
| 69965 | 32055962 | 32059172 | 241878 |
| 69966 | 32128013 | 32129027 | 183530 |
| 69967 | 32145041 | 32146509 | 183530 |
| 69968 | 32148577 | 32151295 | 100150 |
| 69969 | 32210664 | 32211962 | 100150 |
| 69970 | 32224594 | 32227490 | 100150 |
| 69971 | 32339132 | 32342295 | 128245, 128254 |
| 69972 | 32365917 | 32367104 | 230866, 227519 |
| 69973 | 32369131 | 32370813 | 230866 |
| 69974 | 32415969 | 32416569 | |
| 69975 | 32474975 | 32475837 | 100170 |
| 69976 | 32533801 | 32534368 | |
| 69977 | 32806563 | 32809014 | 100220 |
| 69978 | 32817351 | 32818029 | 184459 |
| 69979 | 32822936 | 32823284 | 184459 |
| 69980 | 32869202 | 32872351 | 100225 |
| 69981 | 32935842 | 32936499 | 185666 |
| 69982 | 32953824 | 32955643 | 185666 |
| 69983 | 32975614 | 32976435 | 185666 |
| 69984 | 32979110 | 32980604 | 236054, 185666 |
| 69985 | 33014193 | 33015160 | 185666 |
| 69986 | 33015332 | 33016285 | 185666 |
| 69987 | 33019462 | 33020383 | 185666 |
| 69988 | 33025015 | 33025650 | 185666 |
| 69989 | 33039873 | 33041392 | 185666 |
| 69990 | 33091099 | 33092372 | 185666 |
| 69991 | 33146976 | 33147649 | 185666 |
| 69992 | 33150252 | 33151368 | 185666 |
| 69993 | 33175575 | 33176889 | 185666 |
| 69994 | 33197053 | 33198830 | 100234, 185666 |
| 69995 | 33328088 | 33328796 | 185666 |
| 69996 | 33363006 | 33364100 | 185666 |
| 69997 | 33375560 | 33376080 | 185666 |
| 69998 | 33667515 | 33668973 | 133424 |
| 69999 | 33733545 | 33734018 | 133424 |
| 70000 | 33750837 | 33751373 | 133424 |
| 70001 | 33775743 | 33776295 | 133424 |
| 70002 | 33777914 | 33779275 | 133424 |
| 70003 | 33789261 | 33790150 | 133424 |
| 70004 | 33935863 | 33936830 | 133424 |
| 70005 | 33959314 | 33961145 | 133424 |
| 70006 | 33973847 | 33975077 | 133424 |
| 70007 | 34052637 | 34053670 | 133424 |
| 70008 | 34122007 | 34122713 | 224973, 133424 |
| 70009 | 34153674 | 34154431 | 133424 |
| 70010 | 34181317 | 34182450 | 133424 |
| 70011 | 34260542 | 34261669 | 133424 |
| 70012 | 34262951 | 34263733 | 133424 |
| 70013 | 34270169 | 34271096 | 133424 |
| 70014 | 34315327 | 34316563 | 133424 |
| 70015 | 34316720 | 34318665 | 273082, 133424 |
| 70016 | 34364870 | 34365498 | |
| 70017 | 34396475 | 34398627 | |
| 70018 | 34511099 | 34512391 | 236052 |
| 70019 | 34632392 | 34632977 | |
| 70020 | 34690612 | 34691624 | |
| 70021 | 35253839 | 35254629 | |
| 70022 | 35653118 | 35654674 | 100281 |
| 70023 | 35694983 | 35698533 | 100284, 273176 |
| 70024 | 35706281 | 35712343 | 100284 |
| 70025 | 35724391 | 35724997 | 100284 |
| 70026 | 35726200 | 35727158 | 100284 |
| 70027 | 35757047 | 35757549 | |
| 70028 | 35767510 | 35769428 | |
| 70029 | 35772166 | 35778413 | 100292 |
| 70030 | 35793129 | 35794143 | |
| 70031 | 35795453 | 35796852 | 100297 |
| 70032 | 35826706 | 35827477 | |
| 70033 | 35931552 | 35932703 | |
| 70034 | 35935715 | 35936811 | |
| 70035 | 35985208 | 35985911 | |
| 70036 | 36023070 | 36024410 | 268818, 198125 |
| 70037 | 36025336 | 36026522 | 268818, 198125 |
| 70038 | 36038197 | 36038981 | |
| 70039 | 36042529 | 36043551 | |
| 70040 | 36043981 | 36046342 | 221963 |
| 70041 | 36073511 | 36074010 | |
| 70042 | 36106493 | 36107013 | |
| 70043 | 36138088 | 36139318 | 100320 |
| 70044 | 36213813 | 36214357 | 100320 |
| 70045 | 36214870 | 36216067 | 100320 |
| 70046 | 36220189 | 36220867 | 100320 |
| 70047 | 36230798 | 36233289 | 100320 |
| 70048 | 36289468 | 36290220 | 100320 |
| 70049 | 36317574 | 36319405 | 100320 |
| 70050 | 36345244 | 36345732 | 100320 |
| 70051 | 36422744 | 36425603 | 100320 |
| 70052 | 36426173 | 36427035 | |
| 70053 | 36461052 | 36462927 | |
| 70054 | 36519170 | 36520496 | |
| 70055 | 36539418 | 36540110 | 128284 |
| 70056 | 36551192 | 36554941 | 128284 |
| 70057 | 36555313 | 36557254 | 128284 |
| 70058 | 36558181 | 36560065 | 128284 |
| 70059 | 36560199 | 36563181 | 128284 |
| 70060 | 36633512 | 36634504 | 128335 |
| 70061 | 36634519 | 36636144 | 128335 |
| 70062 | 36648864 | 36650237 | 100342 |
| 70063 | 36659271 | 36659737 | 100342 |
| 70064 | 36673432 | 36674249 | |
| 70065 | 36675254 | 36675931 | |
| 70066 | 36681191 | 36681765 | 100345 |
| 70067 | 36724799 | 36726096 | 100345 |
| 70068 | 36726395 | 36728741 | 100345 |
| 70069 | 36746584 | 36749322 | 100345 |
| 70070 | 36756847 | 36758521 | 100345 |
| 70071 | 36758550 | 36760027 | 100345 |
| 70072 | 36760232 | 36761427 | 100345 |
| 70073 | 36764472 | 36765203 | 100345 |
| 70074 | 36765800 | 36766860 | 100345 |
| 70075 | 36770591 | 36771890 | 100345 |
| 70076 | 36779168 | 36779702 | 100345 |
| 70077 | 36780642 | 36785910 | 223695, 100345 |
| 70078 | 36790639 | 36792798 | 223695 |
| 70079 | 36792849 | 36794454 | |
| 70080 | 36805595 | 36806860 | |
| 70081 | 36815241 | 36816503 | |
| 70082 | 36835533 | 36836544 | |
| 70083 | 36847686 | 36850430 | 228719 |
| 70084 | 36850479 | 36852199 | 228719 |
| 70085 | 36855558 | 36864568 | 100348 |
| 70086 | 36876446 | 36878600 | 100348 |
| 70087 | 36924385 | 36926997 | 100353 |
| 70088 | 37055254 | 37057088 | 166862 |
| 70089 | 37171517 | 37172968 | 100360 |
| 70090 | 37177566 | 37178583 | |
| 70091 | 37269514 | 37270394 | 100365 |
| 70092 | 37408006 | 37408833 | 128311 |
| 70093 | 37413363 | 37413936 | 128311 |
| 70094 | 37414274 | 37416773 | 128309, 128311 |
| 70095 | 37419506 | 37421927 | 128309 |
| 70096 | 37426662 | 37431288 | |
| 70097 | 37439685 | 37440477 | |
| 70098 | 37504821 | 37506196 | 187045 |
| 70099 | 37508227 | 37508929 | |
| 70100 | 37522505 | 37523802 | 100385 |
| 70101 | 37563932 | 37564352 | 100385, 235237 |
| 70102 | 37574041 | 37575796 | 235237 |
| 70103 | 37591965 | 37596514 | 133466 |
| 70104 | 37702490 | 37704032 | 100055 |
| 70105 | 37716978 | 37717752 | |
| 70106 | 37719329 | 37721029 | |
| 70107 | 37722684 | 37723203 | |
| 70108 | 37850247 | 37851899 | |
| 70109 | 37896588 | 37900424 | 100065 |
| 70110 | 37914253 | 37915901 | 100065 |
| 70111 | 37937710 | 37938411 | |
| 70112 | 37941088 | 37943353 | |
| 70113 | 37953368 | 37954025 | |
| 70114 | 37955317 | 37960390 | 128283 |
| 70115 | 37962805 | 37964000 | 128283 |
| 70116 | 37965446 | 37968315 | 100079 |
| 70117 | 37970350 | 37971058 | 100079 |
| 70118 | 38002994 | 38006339 | 100083 |
| 70119 | 38021905 | 38022665 | 100083 |
| 70120 | 38043815 | 38044294 | 100092, 233360 |
| 70121 | 38054085 | 38056241 | 241360, 233360, 100092 |
| 70122 | 38070806 | 38073535 | 100097 |
| 70123 | 38080709 | 38081429 | 100101 |
| 70124 | 38082010 | 38083409 | 100101 |
| 70125 | 38127765 | 38128400 | 100106, 100101 |
| 70126 | 38141707 | 38143606 | 100106, 100101 |
| 70127 | 38147272 | 38149570 | 100106, 100101 |
| 70128 | 38154638 | 38155331 | 100106, 100101 |
| 70129 | 38198104 | 38204341 | 100116, 189060 |
| 70130 | 38212420 | 38213015 | 100116 |
| 70131 | 38213331 | 38214126 | |
| 70132 | 38227771 | 38228495 | 100124 |
| 70133 | 38239570 | 38241160 | 207945, 100124 |
| 70134 | 38244439 | 38246385 | 100129, 100124 |
| 70135 | 38292085 | 38293055 | |
| 70136 | 38300097 | 38303521 | 100139 |
| 70137 | 38329425 | 38330436 | 100139 |
| 70138 | 38349503 | 38350724 | 128346, 100142 |
| 70139 | 38377350 | 38378904 | 100142, 100146 |
| 70140 | 38452644 | 38454213 | 233739, 100151 |
| 70141 | 38503517 | 38504405 | 128298 |
| 70142 | 38558752 | 38561105 | 184381 |
| 70143 | 38573499 | 38578597 | 184381 |
| 70144 | 38583341 | 38583983 | 184381 |
| 70145 | 38587905 | 38589188 | 184381 |
| 70146 | 38589559 | 38590276 | 184381 |
| 70147 | 38597068 | 38600006 | 185022, 184381 |
| 70148 | 38609436 | 38610961 | 185022 |
| 70149 | 38613879 | 38616239 | 198792 |
| 70150 | 38668086 | 38669671 | 198792 |
| 70151 | 38705445 | 38706445 | 213923 |
| 70152 | 38711585 | 38714087 | 213923 |
| 70153 | 38797672 | 38798211 | |
| 70154 | 38818343 | 38820871 | 228620 |
| 70155 | 38863466 | 38864535 | 100196 |
| 70156 | 38886520 | 38887465 | 100201 |
| 70157 | 38900167 | 38903325 | 100201 |
| 70158 | 38966315 | 38967102 | |
| 70159 | 39002419 | 39002903 | 184949 |
| 70160 | 39010478 | 39011596 | 184949 |
| 70161 | 39051999 | 39053750 | 100211, 184949 |
| 70162 | 39077463 | 39078917 | 100216, 228274 |
| 70163 | 39095561 | 39097964 | 100221 |
| 70164 | 39100865 | 39103818 | 100226 |
| 70165 | 39129263 | 39130690 | 203583, 100226 |
| 70166 | 39143539 | 39144876 | 225450, 100242 |
| 70167 | 39149334 | 39153091 | 100242 |
| 70168 | 39189415 | 39190387 | 100246, 100242 |
| 70169 | 39267455 | 39269526 | 183741 |
| 70170 | 39320090 | 39320997 | 235209 |
| 70171 | 39377735 | 39379137 | 179750 |
| 70172 | 39379998 | 39381555 | 179750 |
| 70173 | 39492980 | 39493767 | 100298 |
| 70174 | 39541314 | 39542670 | 100307 |
| 70175 | 39547038 | 39549242 | 100307 |
| 70176 | 39570019 | 39573004 | |
| 70177 | 39635464 | 39636076 | 100311 |
| 70178 | 39638361 | 39641204 | 100311 |
| 70179 | 39647838 | 39652458 | |
| 70180 | 39685260 | 39689128 | 224794 |
| 70181 | 39699282 | 39699775 | |
| 70182 | 39699984 | 39700750 | |
| 70183 | 39704243 | 39706110 | |
| 70184 | 39706454 | 39707502 | |
| 70185 | 39712276 | 39712884 | 100316 |
| 70186 | 39713413 | 39716768 | 263764, 100316 |
| 70187 | 39795134 | 39796618 | 100324 |
| 70188 | 39897509 | 39899677 | 100335 |
| 70189 | 39914793 | 39917831 | 128272 |
| 70190 | 39918636 | 39919715 | 128272 |
| 70191 | 39925094 | 39925737 | 187051 |
| 70192 | 39927759 | 39929352 | 187051 |
| 70193 | 40364474 | 40365078 | 100351 |
| 70194 | 40386890 | 40387701 | |
| 70195 | 40392720 | 40393197 | 133477 |
| 70196 | 40394875 | 40396304 | 133477 |
| 70197 | 40439000 | 40442143 | 100354, 261202, 133477 |
| 70198 | 40503125 | 40504492 | 100354 |
| 70199 | 40551912 | 40552806 | 100354 |
| 70200 | 40572806 | 40576011 | 100354 |
| 70201 | 40577150 | 40578112 | 100354 |
| 70202 | 40578961 | 40580046 | 100354 |
| 70203 | 40581801 | 40582579 | 100354 |
| 70204 | 40600355 | 40600833 | 100354 |
| 70205 | 40653772 | 40655497 | 100354 |
| 70206 | 40671280 | 40673340 | 100354 |
| 70207 | 40741490 | 40743685 | 239900 |
| 70208 | 40766325 | 40767517 | 100359, 239900 |
| 70209 | 40892604 | 40896562 | 196588 |
| 70210 | 40903423 | 40910474 | 196588 |
| 70211 | 40914905 | 40916331 | 196588 |
| 70212 | 40923795 | 40924378 | 252124, 196588 |
| 70213 | 41031399 | 41033908 | 196588 |
| 70214 | 41040601 | 41042977 | |
| 70215 | 41074158 | 41075226 | 128285 |
| 70216 | 41078856 | 41079516 | |
| 70217 | 41107441 | 41108050 | |
| 70218 | 41188392 | 41188875 | 100372 |
| 70219 | 41196236 | 41197010 | 100372 |
| 70220 | 41214557 | 41215866 | 100372 |
| 70221 | 41222491 | 41223993 | 100380 |
| 70222 | 41251810 | 41253942 | 196236, 100380 |
| 70223 | 41346365 | 41348837 | 100387, 196236 |
| 70224 | 41417272 | 41419076 | |
| 70225 | 41486717 | 41489946 | 100393, 221160 |
| 70226 | 41592515 | 41593916 | 231993 |
| 70227 | 41619173 | 41620118 | 100395 |
| 70228 | 41630858 | 41631489 | 100399 |
| 70229 | 41633437 | 41634613 | 100399 |
| 70230 | 41643672 | 41644204 | 100401 |
| 70231 | 41680758 | 41683399 | 100401 |
| 70232 | 41683774 | 41685526 | 269104 |
| 70233 | 41686220 | 41687467 | |
| 70234 | 41696927 | 41699330 | 100403 |
| 70235 | 41704303 | 41705106 | 100403 |
| 70236 | 41739914 | 41740844 | 100403 |
| 70237 | 41743222 | 41744110 | 100403 |
| 70238 | 41748003 | 41748394 | 100403 |
| 70239 | 41755944 | 41756641 | 100403 |
| 70240 | 41762953 | 41763712 | 167074 |
| 70241 | 41775865 | 41778852 | 167074 |
| 70242 | 41805864 | 41806751 | |
| 70243 | 41808554 | 41811314 | |
| 70244 | 41813349 | 41814958 | |
| 70245 | 41840395 | 41845083 | 183864 |
| 70246 | 41863798 | 41866160 | 100412, 100410 |
| 70247 | 41874970 | 41876121 | 100412 |
| 70248 | 41895422 | 41896491 | 100412 |
| 70249 | 41939628 | 41940853 | 100413 |
| 70250 | 41944884 | 41946002 | |
| 70251 | 41953577 | 41954326 | |
| 70252 | 41961421 | 41962066 | 172346 |
| 70253 | 41983371 | 41984312 | 100417 |
| 70254 | 41984439 | 41986424 | 100417 |
| 70255 | 42009327 | 42009872 | 100418 |
| 70256 | 42014939 | 42018511 | 196419, 100418 |
| 70257 | 42062562 | 42063156 | |
| 70258 | 42083471 | 42085260 | 184208, 100138 |
| 70259 | 42195468 | 42197080 | 100147 |
| 70260 | 42227842 | 42230512 | 198911, 184068 |
| 70261 | 42230523 | 42231302 | 184068, 198911 |
| 70262 | 42237304 | 42237723 | 198911 |
| 70263 | 42281482 | 42282205 | 198911 |
| 70264 | 42298949 | 42299623 | 198911 |
| 70265 | 42394107 | 42395664 | 183066, 100167 |
| 70266 | 42466574 | 42467487 | 198951 |
| 70267 | 42474541 | 42476908 | 183172, 177096 |
| 70268 | 42485674 | 42487786 | 237037, 184983, 270083 |
| 70269 | 42525023 | 42525882 | 237037, 100197 |
| 70270 | 42568214 | 42570044 | 100207 |
| 70271 | 42665017 | 42666757 | 182057, 100207 |
| 70272 | 42709626 | 42710813 | 100207 |
| 70273 | 42752608 | 42754554 | |
| 70274 | 42756223 | 42756886 | |
| 70275 | 42764247 | 42765755 | 229891 |
| 70276 | 42833405 | 42834440 | 230107 |
| 70277 | 42839556 | 42840731 | 230107 |
| 70278 | 43009722 | 43012094 | 270022, 100227 |
| 70279 | 43037764 | 43038437 | 100243 |
| 70280 | 43041603 | 43043350 | 100243 |
| 70281 | 43044000 | 43046208 | 100243 |
| 70282 | 43048530 | 43049331 | |
| 70283 | 43069802 | 43070417 | |
| 70284 | 43084169 | 43084959 | |
| 70285 | 43093227 | 43094440 | 128274 |
| 70286 | 43150652 | 43151210 | |
| 70287 | 43200940 | 43202410 | 242247 |
| 70288 | 43251660 | 43254482 | 242247, 100266 |
| 70289 | 43258041 | 43258715 | 100266 |
| 70290 | 43276757 | 43279592 | 100266 |
| 70291 | 43280947 | 43282303 | 100266 |
| 70292 | 43282552 | 43284926 | 100266 |
| 70293 | 43301141 | 43302514 | 100266 |
| 70294 | 43309646 | 43310378 | 100266 |
| 70295 | 43314547 | 43319302 | 100266 |
| 70296 | 43325257 | 43327790 | 100266 |
| 70297 | 43334680 | 43337490 | 100266 |
| 70298 | 43354640 | 43357133 | 100266 |
| 70299 | 43365932 | 43366497 | 100266 |
| 70300 | 43389338 | 43392261 | 100266 |
| 70301 | 43399436 | 43401245 | 100266 |
| 70302 | 43409957 | 43411788 | 100266 |
| 70303 | 43474482 | 43474860 | 100271 |
| 70304 | 43521529 | 43523799 | 100290 |
| 70305 | 43538583 | 43539802 | 100294 |
| 70306 | 43547232 | 43548631 | 100300 |
| 70307 | 43581796 | 43583373 | 100304 |
| 70308 | 43583461 | 43584405 | |
| 70309 | 43629538 | 43630156 | 234892, 159307 |
| 70310 | 43631571 | 43635527 | 234892, 159307 |
| 70311 | 43659009 | 43661098 | 159307 |
| 70312 | 43689098 | 43691068 | 159307 |
| 70313 | 43741035 | 43742873 | |
| 70314 | 43774014 | 43774850 | |
| 70315 | 43786889 | 43787911 | |
| 70316 | 43792803 | 43793505 | |
| 70317 | 43795234 | 43797177 | 236117 |
| 70318 | 43831937 | 43832879 | 186732 |
| 70319 | 43869989 | 43871366 | 186732 |
| 70320 | 44207414 | 44209082 | 261251, 186976 |
| 70321 | 44219176 | 44220119 | |
| 70322 | 44278781 | 44279414 | 100341 |
| 70323 | 44292852 | 44295886 | |
| 70324 | 44314434 | 44318797 | |
| 70325 | 44318810 | 44323121 | 100344 |
| 70326 | 44323906 | 44324775 | 100344 |
| 70327 | 44350484 | 44353047 | 224856, 100347, 100344 |
| 70328 | 44419401 | 44420921 | 188677 |
| 70329 | 44433317 | 44434284 | 188677 |
| 70330 | 44893233 | 44894381 | 188636 |
| 70331 | 45080507 | 45081012 | 186654 |
| 70332 | 45093164 | 45093840 | 186654 |
| 70333 | 45097952 | 45099262 | 248405, 241484, 186654 |
| 70334 | 45109897 | 45111119 | 248405, 186654, 241484 |
| 70335 | 45131472 | 45132278 | 248405, 186654, 241484 |
| 70336 | 45554212 | 45556317 | 226328 |
| 70337 | 45558815 | 45560928 | 093000, 226328 |
| 70338 | 45636950 | 45637417 | |
| 70339 | 45640645 | 45644028 | |
| 70340 | 45644495 | 45645686 | |
| 70341 | 45659430 | 45660525 | 273287 |
| 70342 | 45705152 | 45706910 | 100376 |
| 70343 | 45809245 | 45809979 | 128408, 077935 |
| 70344 | 45879775 | 45880338 | |
| 70345 | 46001206 | 46001637 | 238120 |
| 70346 | 46065187 | 46066525 | |
| 70347 | 46066667 | 46069540 | 130638 |
| 70348 | 46083806 | 46085929 | 130638 |
| 70349 | 46103021 | 46107325 | 130638 |
| 70350 | 46115911 | 46118986 | 130638 |
| 70351 | 46121812 | 46122901 | 130638 |
| 70352 | 46138280 | 46140713 | 130638 |
| 70353 | 46180977 | 46181817 | 130638 |
| 70354 | 46194183 | 46195874 | 130638 |
| 70355 | 46213943 | 46215207 | 130638 |
| 70356 | 46255518 | 46256232 | |
| 70357 | 46402205 | 46403579 | |
| 70358 | 46408493 | 46411244 | 273145 |
| 70359 | 46422699 | 46424197 | |
| 70360 | 46428413 | 46428869 | |
| 70361 | 46431530 | 46433128 | |
| 70362 | 46439472 | 46441617 | 231711 |
| 70363 | 46448477 | 46450943 | 273289, 197182, 241990, |
| | | | 182257 |
| 70364 | 46464248 | 46470967 | 235159, 197182 |
| 70365 | 46474013 | 46474816 | 197182 |
| 70366 | 46480494 | 46482062 | 197182 |
| 70367 | 46483522 | 46486702 | 197182 |
| 70368 | 46499615 | 46504223 | 197182 |
| 70369 | 46512064 | 46513053 | |
| 70370 | 46521431 | 46523646 | |
| 70371 | 46526822 | 46527733 | |
| 70372 | 46527822 | 46529584 | |
| 70373 | 46542825 | 46543910 | |
| 70374 | 46544248 | 46548318 | 186951 |
| 70375 | 46558044 | 46559292 | 186951 |
| 70376 | 46589631 | 46590429 | 186951 |
| 70377 | 46591040 | 46592758 | 186951 |
| 70378 | 46620144 | 46620730 | 186951 |
| 70379 | 46645423 | 46647474 | 205643 |
| 70380 | 46663133 | 46666318 | 075234 |
| 70381 | 46691761 | 46692798 | 075218 |
| 70382 | 46730798 | 46732865 | 100416 |
| 70383 | 46766330 | 46767442 | 075275 |
| 70384 | 46772419 | 46776368 | 075275 |
| 70385 | 46849027 | 46849741 | 075275 |
| 70386 | 46942375 | 46944284 | 234869 |
| 70387 | 46959114 | 46960245 | |
| 70388 | 46970747 | 46974555 | 075240 |
| 70389 | 47034598 | 47035246 | 075240 |
| 70390 | 47044795 | 47046686 | 075240 |
| 70391 | 47047514 | 47049332 | 075240 |
| 70392 | 47121918 | 47123176 | 100422 |
| 70393 | 47126836 | 47128398 | 100422 |
| 70394 | 47133161 | 47135637 | 100422 |
| 70395 | 47136566 | 47137525 | |
| 70396 | 47140148 | 47141088 | |
| 70397 | 47144364 | 47147857 | |
| 70398 | 47148669 | 47151250 | |
| 70399 | 47154410 | 47155516 | |
| 70400 | 47156974 | 47159677 | 260708, 054611 |
| 70401 | 47169569 | 47170965 | 054611 |
| 70402 | 47194302 | 47195129 | 054611 |
| 70403 | 47210857 | 47212840 | 054611 |
| 70404 | 47259919 | 47260999 | 054611 |
| 70405 | 47329927 | 47332825 | 054611 |
| 70406 | 47380301 | 47382217 | 054611 |
| 70407 | 47490582 | 47491082 | 054611 |
| 70408 | 47524859 | 47526724 | 054611 |
| 70409 | 48765656 | 48766860 | |
| 70410 | 48792254 | 48793399 | |
| 70411 | 48801106 | 48802745 | |
| 70412 | 50206476 | 50206874 | 100425 |
| 70413 | 50219540 | 50222315 | 100425 |
| 70414 | 50246202 | 50248691 | 100426 |
| 70415 | 50311201 | 50313232 | 184164, 182858 |
| 70416 | 50327699 | 50329365 | |
| 70417 | 50329429 | 50330391 | |
| 70418 | 50344921 | 50346419 | |
| 70419 | 50347548 | 50348474 | |
| 70420 | 50348623 | 50350221 | |
| 70421 | 50352664 | 50357773 | 198355 |
| 70422 | 50359938 | 50365218 | |
| 70423 | 50585094 | 50585723 | 073146 |
| 70424 | 50607500 | 50608963 | |
| 70425 | 50623465 | 50625412 | 170638 |
| 70426 | 50628504 | 50630113 | 170638 |
| 70427 | 50633243 | 50633824 | 170638 |
| 70428 | 50638571 | 50641285 | 073169, 273253 |
| 70429 | 50642988 | 50645514 | 272836, 073169 |
| 70430 | 50682125 | 50684622 | 100429, 188130, 128159 |
| 70431 | 50688983 | 50689968 | 100429, 188130 |
| 70432 | 50695942 | 50697115 | 188130 |
| 70433 | 50720652 | 50721421 | 196576 |
| 70434 | 50731521 | 50733119 | 196576 |
| 70435 | 50735033 | 50736225 | 196576 |
| 70436 | 50737157 | 50739508 | 196576 |
| 70437 | 50740339 | 50741644 | 196576 |
| 70438 | 50741680 | 50746307 | 196576 |
| 70439 | 50764725 | 50766661 | 205593 |
| 70440 | 50780593 | 50782408 | 100239 |
| 70441 | 50825335 | 50826716 | 100239 |
| 70442 | 50837576 | 50839456 | 100239 |
| 70443 | 50912494 | 50914346 | 100241 |
| 70444 | 50919677 | 50921155 | 128165 |
| 70445 | 50945421 | 50947367 | 025770, 100258 |
| 70446 | 50962629 | 50966045 | 025708, 130489, 272821 |
| 70447 | 50967446 | 50971518 | 177989, 025708 |
| 70448 | 50976689 | 50979996 | |
| 70449 | 50982232 | 50983536 | |
| 70450 | 51000976 | 51002812 | 217442 |
| 70451 | 51007030 | 51008061 | 254413, 205560 |
| 70452 | 51020165 | 51022000 | 205559, 254413, 100288 |
| 70453 | 51051827 | 51052689 | 008735 |
| 70454 | 51058591 | 51059486 | |
| 70455 | 51065683 | 51067058 | 100299 |
| 70456 | 51080186 | 51080733 | |
| 70457 | 51137892 | 51139961 | 251322 |

The locations of ODSCs on Chromosome X are shown in Table 65.

TABLE 65
ODSC Locations on Chromosome X

| SEQ ID NO. | Start | End | Overlapping Gene(s) |
|---|---|---|---|
| 70458 | 2746382 | 2747751 | 056998 |
| 70459 | 2816393 | 2817384 | |
| 70460 | 2817590 | 2818055 | |
| 70461 | 2819243 | 2821856 | |
| 70462 | 2844877 | 2848178 | 006756 |
| 70463 | 2881311 | 2882884 | 157399 |
| 70464 | 2958859 | 2959466 | |
| 70465 | 3631165 | 3631891 | 183943 |
| 70466 | 3732223 | 3735203 | |
| 70467 | 4628424 | 4629310 | |
| 70468 | 6300318 | 6300802 | |
| 70469 | 6972280 | 6973289 | 130021 |
| 70470 | 6979451 | 6981288 | 130021 |
| 70471 | 7008477 | 7009862 | 130021 |
| 70472 | 7038381 | 7039024 | 130021 |
| 70473 | 7050265 | 7050844 | 130021 |
| 70474 | 7065023 | 7066570 | 264268, 130021 |
| 70475 | 7076569 | 7077645 | |
| 70476 | 7739755 | 7740806 | |
| 70477 | 7894262 | 7896568 | 006757 |
| 70478 | 8815984 | 8818110 | |
| 70479 | 8835609 | 8837623 | 229012 |
| 70480 | 8880962 | 8882514 | |
| 70481 | 9236828 | 9237721 | 228543 |
| 70482 | 9309589 | 9311158 | |
| 70483 | 9424438 | 9425166 | |
| 70484 | 9430792 | 9434494 | 101849 |
| 70485 | 9460953 | 9461363 | 101849 |
| 70486 | 9524749 | 9526228 | 101849 |
| 70487 | 9530693 | 9531873 | 101849 |
| 70488 | 9534101 | 9534694 | 101849 |
| 70489 | 9572477 | 9573330 | 101849 |
| 70490 | 9754382 | 9755732 | 146950 |
| 70491 | 9775819 | 9776348 | 146950 |
| 70492 | 9981985 | 9983582 | |
| 70493 | 9998522 | 10002971 | 225076, 047644 |
| 70494 | 10007803 | 10009317 | 047644 |
| 70495 | 10045196 | 10046195 | 047644 |
| 70496 | 10069156 | 10069915 | 047644 |
| 70497 | 10124074 | 10125902 | 073464 |
| 70498 | 10136338 | 10136942 | 073464 |
| 70499 | 10141152 | 10142098 | 073464 |
| 70500 | 10504353 | 10505853 | 101871 |
| 70501 | 10546950 | 10547674 | 101871 |
| 70502 | 10550227 | 10550746 | 101871 |
| 70503 | 10577497 | 10578958 | 101871 |
| 70504 | 10722998 | 10724809 | 101871 |
| 70505 | 10737165 | 10738897 | 101871 |
| 70506 | 10918644 | 10920720 | 234129 |
| 70507 | 10939848 | 10941643 | 234129 |
| 70508 | 10957473 | 10958176 | 234129 |
| 70509 | 11118358 | 11119325 | 234129 |
| 70510 | 11129160 | 11130232 | 004961, 234129 |
| 70511 | 11286105 | 11286639 | 047648 |
| 70512 | 11359351 | 11360335 | 047648 |
| 70513 | 11455517 | 11457335 | 047648 |
| 70514 | 11527766 | 11528411 | 047648 |
| 70515 | 11775239 | 11777508 | 005302 |
| 70516 | 12797586 | 12798148 | |
| 70517 | 12809135 | 12810163 | 101911 |
| 70518 | 12992348 | 12995026 | 205542 |
| 70519 | 13006756 | 13007871 | |
| 70520 | 13046562 | 13048993 | |
| 70521 | 13076958 | 13079511 | |
| 70522 | 13119084 | 13120349 | |
| 70523 | 13127708 | 13130076 | |
| 70524 | 13136677 | 13140063 | |
| 70525 | 13140793 | 13142757 | |
| 70526 | 13178928 | 13183922 | |
| 70527 | 13241791 | 13243079 | |
| 70528 | 13414506 | 13418203 | 226434 |
| 70529 | 13511518 | 13514026 | |
| 70530 | 13670359 | 13672130 | 176896 |
| 70531 | 13687209 | 13688191 | 176896 |
| 70532 | 13706823 | 13708406 | 123595 |
| 70533 | 13751897 | 13753749 | 046651, 196459 |
| 70534 | 14046841 | 14048861 | 046647 |
| 70535 | 14917722 | 14918459 | 130150 |
| 70536 | 15287614 | 15288756 | 102048 |
| 70537 | 15635759 | 15638339 | 225833 |
| 70538 | 15692776 | 15694583 | 186312 |
| 70539 | 15872064 | 15874034 | 182287 |
| 70540 | 16178237 | 16179326 | 238178 |
| 70541 | 16229881 | 16230912 | |
| 70542 | 16621971 | 16623567 | 047230 |
| 70543 | 16736788 | 16738841 | 169895 |
| 70544 | 16757682 | 16758201 | 169895 |
| 70545 | 16761740 | 16762444 | 169895 |
| 70546 | 16803763 | 16805428 | 086712 |
| 70547 | 16825568 | 16826572 | 086712 |
| 70548 | 16845160 | 16847245 | 086712 |
| 70549 | 16856372 | 16857265 | 086712 |
| 70550 | 16887962 | 16889691 | 102054 |
| 70551 | 16964820 | 16965824 | 169891 |
| 70552 | 17023721 | 17024458 | 169891 |
| 70553 | 17095583 | 17097070 | 169891 |
| 70554 | 17423029 | 17423851 | 188158 |
| 70555 | 17754630 | 17756362 | 047634 |
| 70556 | 17832747 | 17833325 | 131831 |
| 70557 | 17938033 | 17938647 | |
| 70558 | 17985784 | 17986438 | |
| 70559 | 18008349 | 18009324 | 225882 |
| 70560 | 18372090 | 18373374 | 102098 |
| 70561 | 18692508 | 18693950 | |
| 70562 | 18953996 | 18954546 | 044446 |
| 70563 | 18998895 | 18999973 | 044446 |
| 70564 | 19001801 | 19003815 | 044446 |
| 70565 | 19090473 | 19091090 | 173698 |
| 70566 | 19104317 | 19104709 | 173698 |
| 70567 | 19145592 | 19146419 | |
| 70568 | 19361714 | 19363021 | 131828 |
| 70569 | 19758912 | 19759291 | 147010 |
| 70570 | 19764913 | 19765676 | 147010 |
| 70571 | 19869986 | 19871042 | 147010 |
| 70572 | 19904355 | 19906929 | 147010 |
| 70573 | 19924412 | 19924884 | |
| 70574 | 20008742 | 20009611 | |
| 70575 | 20044139 | 20044997 | 184368 |
| 70576 | 20047560 | 20048504 | 184368 |
| 70577 | 20079781 | 20080527 | 184368 |
| 70578 | 20158869 | 20160447 | 173674 |
| 70579 | 20236812 | 20237983 | 177189 |
| 70580 | 20239899 | 20240652 | 177189 |
| 70581 | 20283344 | 20285953 | 177189 |
| 70582 | 20379199 | 20381240 | |
| 70583 | 20534254 | 20535270 | |
| 70584 | 20565420 | 20566464 | |
| 70585 | 21718932 | 21719535 | |
| 70586 | 21760105 | 21760808 | 091482 |
| 70587 | 21816089 | 21819627 | |
| 70588 | 21856955 | 21858700 | 012174 |
| 70589 | 21943801 | 21945649 | |
| 70590 | 21950162 | 21951739 | |
| 70591 | 21958055 | 21959860 | 102172, 271286 |
| 70592 | 22127190 | 22128592 | 102174 |
| 70593 | 23020914 | 23021860 | 233067 |
| 70594 | 23511688 | 23512554 | |
| 70595 | 23522119 | 23523233 | |
| 70596 | 23684903 | 23686410 | 123131 |
| 70597 | 23759927 | 23761839 | 123130 |
| 70598 | 23788214 | 23788656 | |
| 70599 | 23791024 | 23792195 | 233785 |
| 70600 | 23799039 | 23804619 | 130066, 202272, 233785 |
| 70601 | 23821956 | 23822931 | |
| 70602 | 23825268 | 23825956 | |
| 70603 | 23889846 | 23890520 | 184831 |
| 70604 | 23897579 | 23898294 | 184831 |
| 70605 | 23925453 | 23926621 | 165182, 184831 |
| 70606 | 24042098 | 24045054 | 174010 |
| 70607 | 24072093 | 24073937 | 130741 |
| 70608 | 24163707 | 24165004 | 234230 |
| 70609 | 24166190 | 24169278 | 005889, 234230 |
| 70610 | 24208462 | 24209092 | 005889 |
| 70611 | 24711499 | 24712537 | 101868 |
| 70612 | 24996622 | 24997656 | 101868 |
| 70613 | 25541616 | 25542784 | |
| 70614 | 25554303 | 25555004 | |
| 70615 | 28207137 | 28207698 | |
| 70616 | 28869746 | 28871072 | 169306 |
| 70617 | 29299051 | 29300184 | 169306 |
| 70618 | 29560821 | 29561419 | 169306 |
| 70619 | 29679638 | 29681655 | 169306 |
| 70620 | 30300640 | 30303129 | |
| 70621 | 30590081 | 30591164 | 120280 |
| 70622 | 30645833 | 30646357 | |
| 70623 | 30661349 | 30662131 | |
| 70624 | 30667473 | 30668809 | |
| 70625 | 30670387 | 30673691 | 198814 |
| 70626 | 30698669 | 30699298 | 198814 |
| 70627 | 30712049 | 30712872 | 198814 |
| 70628 | 30853418 | 30854077 | 231542, 157625 |
| 70629 | 30893227 | 30895764 | 157625 |
| 70630 | 30906591 | 30908052 | 157625 |
| 70631 | 30973673 | 30974328 | 157625 |
| 70632 | 30975694 | 30976277 | 157625 |
| 70633 | 31239076 | 31239851 | 198947 |
| 70634 | 31245138 | 31245809 | 198947 |
| 70635 | 31368219 | 31369045 | 198947 |
| 70636 | 31374497 | 31375054 | 198947 |
| 70637 | 31414794 | 31415802 | 198947 |
| 70638 | 31850034 | 31851343 | 198947 |
| 70639 | 31907128 | 31908050 | 198947 |
| 70640 | 31911739 | 31912831 | 198947 |
| 70641 | 34422500 | 34423218 | 233571 |
| 70642 | 34675137 | 34676044 | 147027 |
| 70643 | 34902801 | 34903647 | |
| 70644 | 34933285 | 34937254 | |
| 70645 | 35306447 | 35313282 | |
| 70646 | 36927578 | 36928128 | |
| 70647 | 36981539 | 36982351 | |
| 70648 | 37191876 | 37193613 | |
| 70649 | 37208446 | 37209674 | 130962, 250349 |
| 70650 | 37313551 | 37315268 | 130962, 250349 |
| 70651 | 37486849 | 37487453 | 250349, 147036 |
| 70652 | 37544647 | 37545448 | 047597, 250349 |
| 70653 | 37571067 | 37572035 | 250349, 047597 |
| 70654 | 37705991 | 37707124 | 165169, 250349 |
| 70655 | 37746496 | 37747655 | 250349 |
| 70656 | 37817467 | 37819070 | 250349 |
| 70657 | 37920530 | 37921092 | 250349, 147041 |
| 70658 | 37966093 | 37967126 | 250349, 147041 |
| 70659 | 38134316 | 38135626 | 250349, 156313 |
| 70660 | 38186428 | 38187238 | 156313, 250349 |
| 70661 | 38201755 | 38203444 | 250349 |
| 70662 | 38210446 | 38211003 | 250349 |
| 70663 | 38421169 | 38422566 | 250349, 156298 |
| 70664 | 38660113 | 38662165 | 165175, 238123 |
| 70665 | 38662245 | 38666876 | 165175, 238123 |
| 70666 | 38676167 | 38676735 | |
| 70667 | 38691237 | 38691830 | |
| 70668 | 38749932 | 38750677 | |
| 70669 | 38796572 | 38797755 | |
| 70670 | 38798283 | 38798826 | |
| 70671 | 38799693 | 38800338 | |
| 70672 | 38903043 | 38903608 | |
| 70673 | 39500942 | 39501946 | |
| 70674 | 39547841 | 39548685 | |
| 70675 | 39563695 | 39564905 | |
| 70676 | 39592007 | 39592797 | |
| 70677 | 39611799 | 39613405 | |
| 70678 | 39617797 | 39620047 | |
| 70679 | 39696018 | 39696824 | 261435, 263972 |
| 70680 | 39709207 | 39710253 | |
| 70681 | 39766208 | 39768617 | |
| 70682 | 39795583 | 39796909 | |
| 70683 | 39813037 | 39813787 | |
| 70684 | 39871161 | 39874489 | 223486 |
| 70685 | 39915530 | 39916187 | 183337 |
| 70686 | 39941828 | 39942342 | 183337 |
| 70687 | 39942526 | 39943203 | 183337 |
| 70688 | 39954135 | 39957787 | 183337 |
| 70689 | 39963587 | 39969628 | 183337 |
| 70690 | 39991016 | 39992464 | 183337 |
| 70691 | 39999858 | 40000724 | 183337 |
| 70692 | 40011405 | 40013895 | 183337 |
| 70693 | 40013969 | 40015484 | 183337 |
| 70694 | 40017011 | 40018300 | 183337 |
| 70695 | 40032542 | 40036982 | 183337 |
| 70696 | 40333287 | 40334091 | |
| 70697 | 40372237 | 40372787 | |
| 70698 | 40439885 | 40441074 | 182220 |
| 70699 | 40504035 | 40504725 | 185753 |
| 70700 | 40505596 | 40507139 | 185753 |
| 70701 | 40586665 | 40587386 | 180182 |
| 70702 | 40594106 | 40596103 | 234636, 180182 |
| 70703 | 40643591 | 40644713 | |
| 70704 | 40725457 | 40727087 | |
| 70705 | 40804064 | 40804995 | |
| 70706 | 40869118 | 40870514 | |
| 70707 | 40875945 | 40876784 | |
| 70708 | 40881266 | 40882879 | |
| 70709 | 40906735 | 40908174 | |
| 70710 | 40942968 | 40947025 | 124486 |
| 70711 | 41189635 | 41190279 | |
| 70712 | 41190465 | 41195050 | 215301 |
| 70713 | 41240561 | 41241428 | |
| 70714 | 41241703 | 41243883 | |
| 70715 | 41248082 | 41249247 | |
| 70716 | 41300955 | 41302740 | |
| 70717 | 41420169 | 41421054 | 147044 |
| 70718 | 41536483 | 41537093 | 147044 |
| 70719 | 41548920 | 41549600 | 171659, 147044 |
| 70720 | 41620100 | 41621673 | 147044 |
| 70721 | 41638132 | 41639098 | 147044 |
| 70722 | 41682176 | 41683280 | 147044 |
| 70723 | 41694731 | 41695111 | 147044 |
| 70724 | 41765706 | 41766554 | 147044 |
| 70725 | 41781561 | 41784005 | 147044 |
| 70726 | 41952975 | 41953960 | |
| 70727 | 41954804 | 41956024 | |
| 70728 | 42397450 | 42398124 | 231772 |
| 70729 | 42566957 | 42567944 | |
| 70730 | 43084649 | 43085981 | 224294 |
| 70731 | 43448667 | 43450310 | |
| 70732 | 43513594 | 43516193 | 189221 |
| 70733 | 43523395 | 43524836 | 189221 |
| 70734 | 43552711 | 43553943 | 189221 |
| 70735 | 43560689 | 43563595 | 189221 |
| 70736 | 43566599 | 43567796 | 189221 |
| 70737 | 43588164 | 43588663 | 189221 |
| 70738 | 43704792 | 43706284 | 069535 |
| 70739 | 43712462 | 43713419 | 069535 |
| 70740 | 43720054 | 43721952 | 069535 |
| 70741 | 43738225 | 43742142 | 069535 |
| 70742 | 44287794 | 44289198 | |
| 70743 | 44401237 | 44403028 | 069509 |
| 70744 | 44730633 | 44734492 | 147050 |
| 70745 | 44773643 | 44775284 | 147050 |
| 70746 | 44849088 | 44849543 | 147050 |
| 70747 | 44868607 | 44870940 | 147050 |
| 70748 | 45365227 | 45366986 | 229563 |
| 70749 | 45374272 | 45376013 | 229563 |
| 70750 | 45628275 | 45631714 | 270069 |
| 70751 | 45710779 | 45711567 | 231566 |
| 70752 | 45903946 | 45904379 | |
| 70753 | 46064389 | 46064937 | |
| 70754 | 46179668 | 46180378 | |
| 70755 | 46186184 | 46187763 | 236751 |
| 70756 | 46192997 | 46193402 | |
| 70757 | 46305740 | 46307534 | 147121 |
| 70758 | 46404037 | 46406009 | 230844, 251192 |
| 70759 | 46482903 | 46484743 | 065923 |
| 70760 | 46569454 | 46570993 | 065923 |
| 70761 | 46617698 | 46619578 | 065923 |
| 70762 | 46671065 | 46673088 | |
| 70763 | 46675347 | 46676019 | |
| 70764 | 46696074 | 46697150 | 102218 |
| 70765 | 46771269 | 46772906 | 102221 |
| 70766 | 46817386 | 46819173 | 102221 |
| 70767 | 46827956 | 46828542 | 102221 |
| 70768 | 46937352 | 46938677 | 130988 |
| 70769 | 46988331 | 46989620 | |
| 70770 | 47003851 | 47005324 | 182872, 147123 |
| 70771 | 47052482 | 47054341 | 130985 |
| 70772 | 47059732 | 47060873 | 130985 |
| 70773 | 47076930 | 47079299 | 102225 |
| 70774 | 47341786 | 47343519 | 196741, 147124 |
| 70775 | 47400402 | 47401147 | |
| 70776 | 47419649 | 47421147 | 078061 |
| 70777 | 47441455 | 47442087 | 102265, 008056 |
| 70778 | 47504876 | 47505488 | 126767 |
| 70779 | 47509392 | 47510844 | 126767 |
| 70780 | 47517929 | 47519036 | 267064, 126756 |
| 70781 | 48333661 | 48335370 | 068438 |
| 70782 | 48363877 | 48364520 | |
| 70783 | 48379735 | 48381153 | 147155 |
| 70784 | 48397464 | 48398796 | 068354 |
| 70785 | 48432002 | 48434173 | 102317, 204620 |
| 70786 | 48447805 | 48452016 | 101940 |
| 70787 | 48455267 | 48456739 | 101940 |
| 70788 | 48554054 | 48555725 | 101945 |
| 70789 | 48569686 | 48573335 | 232828 |
| 70790 | 48659418 | 48661990 | 094631 |
| 70791 | 48754423 | 48755684 | 102103, 126768 |
| 70792 | 48768411 | 48770102 | 102100 |
| 70793 | 48775662 | 48776745 | 102096 |
| 70794 | 48814085 | 48816910 | 223309, 068308 |
| 70795 | 48857985 | 48859388 | 068400 |
| 70796 | 48876967 | 48877786 | |
| 70797 | 48898825 | 48902126 | 068323 |
| 70798 | 48910794 | 48911901 | 147144 |
| 70799 | 48916291 | 48917673 | 147144 |
| 70800 | 48931436 | 48932006 | 243279, 250232, 196998 |
| 70801 | 48936597 | 48938369 | 250232, 196998 |
| 70802 | 48979297 | 48980622 | 068394 |
| 70803 | 49019988 | 49020819 | 017621 |
| 70804 | 49027805 | 49029250 | 102007 |
| 70805 | 49042180 | 49043362 | 012211 |
| 70806 | 49125139 | 49127450 | 049769 |
| 70807 | 49643109 | 49644205 | 234390 |
| 70808 | 49831943 | 49832527 | 171365 |
| 70809 | 50191496 | 50192008 | 204466 |
| 70810 | 50570312 | 50571621 | |
| 70811 | 50603361 | 50604482 | |
| 70812 | 51485972 | 51487197 | 189369 |
| 70813 | 51636472 | 51638173 | 179222 |
| 70814 | 52894420 | 52895166 | 171402 |
| 70815 | 52949302 | 52951076 | |
| 70816 | 52963643 | 52965148 | |
| 70817 | 53253063 | 53255310 | 126012 |
| 70818 | 53448629 | 53449847 | 158423, 072501 |
| 70819 | 53467308 | 53469105 | |
| 70820 | 53709750 | 53712025 | 086758 |
| 70821 | 53712990 | 53713627 | 086758 |
| 70822 | 54068980 | 54069769 | 172943 |
| 70823 | 54069780 | 54072450 | 172943 |
| 70824 | 54197944 | 54198368 | 184083 |
| 70825 | 54208706 | 54210137 | 184083 |
| 70826 | 54256573 | 54258092 | 196632 |
| 70827 | 54319043 | 54319604 | 196632 |
| 70828 | 54384264 | 54385360 | 196632 |
| 70829 | 54555387 | 54557430 | 130119 |
| 70830 | 54566690 | 54567128 | 130119 |
| 70831 | 54665460 | 54667597 | |
| 70832 | 54733598 | 54734383 | |
| 70833 | 54734405 | 54735577 | |
| 70834 | 54834106 | 54835693 | 102316 |
| 70835 | 55020056 | 55022789 | 158571 |
| 70836 | 55033253 | 55034950 | 169188 |
| 70837 | 55034991 | 55036044 | 158578, 169188 |
| 70838 | 55059223 | 55060614 | |
| 70839 | 55187077 | 55188615 | 182518 |
| 70840 | 55228275 | 55228898 | |
| 70841 | 55239806 | 55240290 | |
| 70842 | 55478124 | 55478963 | 187601 |
| 70843 | 55650806 | 55651320 | 189299 |
| 70844 | 56156954 | 56157591 | |
| 70845 | 56589393 | 56591122 | 188021 |
| 70846 | 56755443 | 56756297 | 204272 |
| 70847 | 56764567 | 56765784 | 204272 |
| 70848 | 56789858 | 56791664 | 204272 |
| 70849 | 56824397 | 56824963 | 204272 |
| 70850 | 57021383 | 57022794 | 204271 |
| 70851 | 57312808 | 57313900 | 165591 |
| 70852 | 57617643 | 57618425 | 198455 |
| 70853 | 57620687 | 57624828 | 198455 |
| 70854 | 57666417 | 57669326 | |
| 70855 | 57669458 | 57672075 | |
| 70856 | 57926911 | 57927973 | |
| 70857 | 62412150 | 62412963 | |
| 70858 | 62570157 | 62571824 | 186767, 235437, 233661 |
| 70859 | 62780271 | 62781443 | 235437 |
| 70860 | 62933055 | 62933914 | 131089 |
| 70861 | 62974112 | 62975419 | 131089 |
| 70862 | 63141399 | 63142557 | |
| 70863 | 63424048 | 63427061 | 184675 |
| 70864 | 63615062 | 63615921 | 102043 |
| 70865 | 64254162 | 64255615 | 126970 |
| 70866 | 64416585 | 64417967 | |
| 70867 | 64753895 | 64755184 | 001497 |
| 70868 | 64814996 | 64817027 | 147065 |
| 70869 | 64820666 | 64822571 | 147065 |
| 70870 | 64862222 | 64865774 | 147065 |
| 70871 | 64871888 | 64872606 | 147065 |
| 70872 | 64886462 | 64889834 | 147065 |
| 70873 | 64893289 | 64894787 | 147065 |
| 70874 | 64925621 | 64926682 | 147065 |
| 70875 | 65948533 | 65949243 | |
| 70876 | 66262994 | 66265312 | |
| 70877 | 66270470 | 66271221 | |
| 70878 | 66319765 | 66321013 | |
| 70879 | 66324937 | 66325734 | |
| 70880 | 66510397 | 66511016 | |
| 70881 | 66515495 | 66516180 | |
| 70882 | 66566460 | 66566885 | |
| 70883 | 66743591 | 66744227 | |
| 70884 | 66755134 | 66756960 | |
| 70885 | 66763012 | 66764592 | 169083 |
| 70886 | 66770484 | 66771661 | 169083 |
| 70887 | 66782853 | 66784288 | 169083 |
| 70888 | 66787849 | 66789537 | 169083 |
| 70889 | 66816585 | 66817544 | 169083 |
| 70890 | 66824894 | 66825907 | 169083 |
| 70891 | 66832023 | 66833023 | 169083 |
| 70892 | 66833417 | 66835169 | 169083 |
| 70893 | 66838974 | 66840216 | 169083 |
| 70894 | 66856273 | 66856825 | 169083 |
| 70895 | 66873851 | 66874882 | 169083 |
| 70896 | 66919932 | 66921096 | 169083 |
| 70897 | 67708331 | 67709157 | |
| 70898 | 67717783 | 67719713 | 181704 |
| 70899 | 67913237 | 67915688 | 130052 |
| 70900 | 67918774 | 67919281 | 130052 |
| 70901 | 68048403 | 68051206 | 090776 |
| 70902 | 68061176 | 68062371 | 090776 |
| 70903 | 68544563 | 68545285 | |
| 70904 | 69353060 | 69354418 | 089289 |
| 70905 | 69653524 | 69654509 | |
| 70906 | 70286942 | 70288949 | 147164 |
| 70907 | 70338137 | 70339269 | 184634 |
| 70908 | 70401149 | 70403034 | |
| 70909 | 70434545 | 70435789 | 169562 |
| 70910 | 70436989 | 70437807 | 169562 |
| 70911 | 70447378 | 70448569 | |
| 70912 | 70473669 | 70474628 | 147130 |
| 70913 | 70502629 | 70504248 | 147140 |
| 70914 | 70584751 | 70587379 | 147133 |
| 70915 | 70751433 | 70754520 | 147162, 147133 |
| 70916 | 71209566 | 71210452 | 204131 |
| 70917 | 71238153 | 71238844 | 204131 |
| 70918 | 71248517 | 71250758 | 204131 |
| 70919 | 71400715 | 71402392 | 102309 |
| 70920 | 71495706 | 71497347 | 198034, 102309 |
| 70921 | 71791955 | 71793326 | 147099 |
| 70922 | 71796322 | 71797338 | |
| 70923 | 71839666 | 71841758 | 067177 |
| 70924 | 73137033 | 73137554 | |
| 70925 | 73157423 | 73158061 | |
| 70926 | 73160575 | 73161141 | |
| 70927 | 73163110 | 73165142 | 225470 |
| 70928 | 73183485 | 73183835 | 230590, 225470, 271199 |
| 70929 | 73461645 | 73463075 | 265727, 271430, 230590 |
| 70930 | 73482020 | 73482822 | 230590 |
| 70931 | 73510138 | 73513520 | 230590 |
| 70932 | 73563694 | 73564287 | |
| 70933 | 73639727 | 73643780 | 147100 |
| 70934 | 73669650 | 73672369 | 147100 |
| 70935 | 73719798 | 73721013 | 147100 |
| 70936 | 73725990 | 73726730 | 147100 |
| 70937 | 73734097 | 73734722 | 147100 |
| 70938 | 73753692 | 73757351 | 147100 |
| 70939 | 73833074 | 73835120 | 131263 |
| 70940 | 74493547 | 74495004 | 094841 |
| 70941 | 76307738 | 76309253 | |
| 70942 | 76340075 | 76340588 | |
| 70943 | 77040877 | 77042016 | 085224 |
| 70944 | 77154465 | 77155821 | 131174 |
| 70945 | 77165673 | 77167060 | 165240, 248503 |
| 70946 | 77359134 | 77360932 | 102144 |
| 70947 | 77908081 | 77910027 | |
| 70948 | 80020622 | 80021132 | 165288 |
| 70949 | 80063115 | 80064206 | 165288 |
| 70950 | 80064576 | 80066434 | 165288 |
| 70951 | 80376383 | 80377954 | 198157 |
| 70952 | 80457032 | 80458071 | 131171, 198157 |
| 70953 | 81588799 | 81589892 | |
| 70954 | 82068562 | 82069216 | |
| 70955 | 83756712 | 83757748 | 259640, 165259 |
| 70956 | 83984447 | 83985148 | |
| 70957 | 84258675 | 84259737 | 155008 |
| 70958 | 84886964 | 84887434 | |
| 70959 | 85247490 | 85249056 | 188419 |
| 70960 | 85294786 | 85295585 | 188419 |
| 70961 | 85624550 | 85625749 | 126733 |
| 70962 | 87815342 | 87816337 | |
| 70963 | 87858979 | 87861203 | |
| 70964 | 93655280 | 93656539 | |
| 70965 | 95687357 | 95689347 | |
| 70966 | 95708028 | 95708740 | |
| 70967 | 95818129 | 95819749 | |
| 70968 | 95939315 | 95941389 | 147202 |
| 70969 | 96415198 | 96415976 | 147202 |
| 70970 | 96654221 | 96657221 | 147202 |
| 70971 | 96700747 | 96701229 | 147202, 236256 |
| 70972 | 96818448 | 96818884 | 147202, 236256 |
| 70973 | 96883662 | 96884995 | 236256 |
| 70974 | 98333191 | 98333812 | |
| 70975 | 98608184 | 98609012 | |
| 70976 | 99869676 | 99870672 | |
| 70977 | 99891037 | 99892113 | 000003 |
| 70978 | 99898695 | 99900194 | 102359 |
| 70979 | 99901248 | 99906431 | 102359 |
| 70980 | 100074844 | 100075766 | 101811 |
| 70981 | 100234932 | 100235967 | 174225 |
| 70982 | 100249403 | 100249982 | |
| 70983 | 100306309 | 100307036 | 188917, 225839 |
| 70984 | 100602109 | 100603981 | 126953 |
| 70985 | 100645597 | 100646930 | 241343, 257529 |
| 70986 | 100661918 | 100664275 | 126945, 102393, 257529 |
| 70987 | 100877647 | 100879118 | 102401, 228275 |
| 70988 | 101186253 | 101187582 | 166432 |
| 70989 | 101853781 | 101855126 | 271147, 270050, 125962 |
| 70990 | 101914449 | 101915138 | 270050, 271147 |
| 70991 | 102508971 | 102510344 | 180964 |
| 70992 | 102617203 | 102617915 | |
| 70993 | 102628572 | 102629144 | |
| 70994 | 102805282 | 102806011 | |
| 70995 | 102806819 | 102807855 | |
| 70996 | 102883510 | 102885201 | 172465, 196507 |
| 70997 | 102940270 | 102943237 | 231154, 123562 |
| 70998 | 103401081 | 103402560 | 176274 |
| 70999 | 103410494 | 103412126 | 123575 |
| 71000 | 103562826 | 103564342 | |
| 71001 | 103810349 | 103811053 | 189108 |
| 71002 | 103903749 | 103905179 | 189108 |
| 71003 | 103907270 | 103909355 | 189108 |
| 71004 | 103994453 | 103995335 | 189108 |
| 71005 | 104098810 | 104100276 | 189108 |
| 71006 | 104146503 | 104147256 | 189108 |
| 71007 | 104166658 | 104167351 | 189108 |
| 71008 | 104212927 | 104213239 | 189108 |
| 71009 | 104261891 | 104263059 | 189108 |
| 71010 | 105213450 | 105213884 | |
| 71011 | 105216783 | 105217658 | |
| 71012 | 105251481 | 105252615 | |
| 71013 | 105282435 | 105283026 | 123561 |
| 71014 | 105288872 | 105289622 | |
| 71015 | 105935595 | 105937574 | 133135 |
| 71016 | 105958877 | 105960036 | 133135 |
| 71017 | 105961740 | 105962424 | 133135 |
| 71018 | 105969229 | 105971147 | 133135 |
| 71019 | 106023469 | 106024997 | 133135 |
| 71020 | 106045368 | 106046506 | 133138 |
| 71021 | 106141661 | 106142287 | 133131 |
| 71022 | 106144434 | 106145553 | 147223, 133131, 165376 |
| 71023 | 106170454 | 106171776 | 133131, 165376 |
| 71024 | 106175239 | 106176345 | 133131 |
| 71025 | 106176399 | 106177383 | 133131 |
| 71026 | 106197531 | 106198729 | 133131 |
| 71027 | 106216195 | 106217146 | 133131 |
| 71028 | 106242586 | 106243960 | 133131 |
| 71029 | 106361192 | 106362328 | 089682 |
| 71030 | 106958707 | 106962085 | 157514 |
| 71031 | 106984906 | 106986630 | 157514 |
| 71032 | 106990617 | 106991450 | 157514 |
| 71033 | 107068791 | 107071351 | 080561 |
| 71034 | 107077467 | 107078411 | 080561 |
| 71035 | 107090188 | 107090794 | 080561 |
| 71036 | 107333368 | 107335729 | 101844, 101843 |
| 71037 | 107518904 | 107519375 | 197565 |
| 71038 | 107550514 | 107551199 | 197565 |
| 71039 | 108297508 | 108298018 | 226958 |
| 71040 | 108779464 | 108781496 | 101888 |
| 71041 | 108975480 | 108976985 | 068366 |
| 71042 | 108983005 | 108983990 | |
| 71043 | 109017788 | 109018413 | |
| 71044 | 109040355 | 109041241 | |
| 71045 | 109073587 | 109075079 | |
| 71046 | 109115217 | 109115889 | |
| 71047 | 109209284 | 109210030 | |
| 71048 | 109245267 | 109246944 | 157600 |
| 71049 | 109247226 | 109248214 | 157600 |
| 71050 | 109279485 | 109280112 | 157600 |
| 71051 | 109324567 | 109326451 | 265584, 157600 |
| 71052 | 109329986 | 109332194 | 157600 |
| 71053 | 109528748 | 109529645 | 101935 |
| 71054 | 109831213 | 109832592 | |
| 71055 | 110595447 | 110596519 | 077279 |
| 71056 | 110596568 | 110597834 | 077279 |
| 71057 | 110703687 | 110704608 | |
| 71058 | 110923965 | 110925586 | 101901 |
| 71059 | 111325848 | 111327619 | 072315 |
| 71060 | 112083387 | 112084174 | 126016 |
| 71061 | 114578998 | 114580843 | |
| 71062 | 114627809 | 114628301 | |
| 71063 | 114793455 | 114797366 | 102024, 271826 |
| 71064 | 115008230 | 115010037 | |
| 71065 | 115084491 | 115085759 | 235244 |
| 71066 | 115125897 | 115126843 | |
| 71067 | 116271788 | 116272691 | |
| 71068 | 116468950 | 116469684 | |
| 71069 | 116509787 | 116510826 | |
| 71070 | 116775865 | 116776689 | |
| 71071 | 116847233 | 116847895 | |
| 71072 | 117125671 | 117126099 | 003096 |
| 71073 | 117250431 | 117251504 | 239500, 003096 |
| 71074 | 117289989 | 117290803 | |
| 71075 | 117479008 | 117482247 | 131725 |
| 71076 | 117760266 | 117760806 | 147251 |
| 71077 | 117789570 | 117790210 | 147251 |
| 71078 | 117858574 | 117859296 | |
| 71079 | 117860265 | 117863004 | 131724 |
| 71080 | 117877435 | 117878369 | 131724 |
| 71081 | 117973053 | 117974300 | 203650 |
| 71082 | 118005379 | 118007317 | 203650 |
| 71083 | 118107761 | 118111563 | 175556 |
| 71084 | 118174887 | 118176455 | |
| 71085 | 118281416 | 118281962 | 250423 |
| 71086 | 118320762 | 118321678 | 227864 |
| 71087 | 118370086 | 118371509 | 101856 |
| 71088 | 118532530 | 118534118 | 077713 |
| 71089 | 118545850 | 118546295 | 077713 |
| 71090 | 118552901 | 118553978 | 077713 |
| 71091 | 118561063 | 118562160 | 077713 |
| 71092 | 118601254 | 118603613 | 005022, 224281 |
| 71093 | 118611375 | 118612856 | |
| 71094 | 118614862 | 118617099 | |
| 71095 | 118636463 | 118638352 | |
| 71096 | 118639685 | 118640497 | |
| 71097 | 118707753 | 118709378 | 077721 |
| 71098 | 118739406 | 118741300 | 186416 |
| 71099 | 118824701 | 118827495 | 125354 |
| 71100 | 118924781 | 118926593 | 198918 |
| 71101 | 118952565 | 118953159 | |
| 71102 | 118986313 | 118987626 | 125351 |
| 71103 | 119004432 | 119006700 | 125356, 125352 |
| 71104 | 119076508 | 119078031 | 101882 |
| 71105 | 119378511 | 119379555 | 233382 |
| 71106 | 119384449 | 119386280 | 177485 |
| 71107 | 119431219 | 119432393 | 125355 |
| 71108 | 119599773 | 119600892 | 005893 |
| 71109 | 119601870 | 119603486 | 005893 |
| 71110 | 119619317 | 119620406 | |
| 71111 | 119646152 | 119647053 | |
| 71112 | 119683155 | 119684909 | 158290 |
| 71113 | 119693965 | 119696183 | 158290 |
| 71114 | 119759199 | 119759658 | 171155 |
| 71115 | 119763244 | 119764349 | 171155 |
| 71116 | 119805491 | 119807089 | |
| 71117 | 119850873 | 119851559 | |
| 71118 | 119886411 | 119887228 | |
| 71119 | 119888591 | 119889688 | |
| 71120 | 120355273 | 120356025 | |
| 71121 | 121699757 | 121700229 | |
| 71122 | 122217711 | 122223839 | |
| 71123 | 122272032 | 122273218 | |
| 71124 | 122317280 | 122319331 | 125675 |
| 71125 | 122474364 | 122475154 | 125675 |
| 71126 | 122724694 | 122725639 | |
| 71127 | 122865596 | 122867269 | 125676 |
| 71128 | 122958503 | 122959676 | |
| 71129 | 122993152 | 122995913 | 101966 |
| 71130 | 123085545 | 123086184 | |
| 71131 | 123087172 | 123087681 | |
| 71132 | 123092866 | 123097152 | 101972, 232412 |
| 71133 | 123220097 | 123220744 | 101972 |
| 71134 | 123619049 | 123619922 | 009694 |
| 71135 | 123727835 | 123729043 | 009694 |
| 71136 | 124338675 | 124340336 | |
| 71137 | 124422475 | 124424248 | |
| 71138 | 127154736 | 127155120 | |
| 71139 | 128063235 | 128063700 | |
| 71140 | 128656470 | 128657783 | 102038 |
| 71141 | 128658987 | 128659565 | |
| 71142 | 128674007 | 128675376 | 122126 |
| 71143 | 128799852 | 128800840 | |
| 71144 | 128882022 | 128882700 | 122121 |
| 71145 | 128910845 | 128911989 | |
| 71146 | 128976505 | 128978147 | 188706 |
| 71147 | 129039498 | 129041074 | 156697, 235189 |
| 71148 | 129090896 | 129092121 | 235189 |
| 71149 | 129298923 | 129300551 | 156709 |
| 71150 | 129402144 | 129403261 | 056277 |
| 71151 | 129816483 | 129817138 | 165675 |
| 71152 | 129847838 | 129848754 | 165675 |
| 71153 | 129861916 | 129862712 | 165675 |
| 71154 | 130036615 | 130037562 | 165675 |
| 71155 | 130533271 | 130534350 | 147255 |
| 71156 | 130574735 | 130575835 | |
| 71157 | 130862216 | 130863916 | 213468 |
| 71158 | 130926088 | 130927824 | 213468 |
| 71159 | 130950703 | 130951638 | 213468 |
| 71160 | 130955726 | 130956963 | 213468 |
| 71161 | 131157053 | 131157818 | 134602 |
| 71162 | 131211666 | 131212363 | 165694 |
| 71163 | 131314722 | 131315899 | |
| 71164 | 131350510 | 131353913 | 232160, 123728 |
| 71165 | 131501888 | 131502786 | 232160 |
| 71166 | 131503519 | 131504202 | 076770, 232160 |
| 71167 | 131535940 | 131536487 | 076770, 232160 |
| 71168 | 131614280 | 131615534 | 076770 |
| 71169 | 131622300 | 131624083 | 076770 |
| 71170 | 131625115 | 131625715 | |
| 71171 | 131666643 | 131667665 | |
| 71172 | 131727467 | 131728961 | |
| 71173 | 133128476 | 133129098 | |
| 71174 | 133940869 | 133942108 | 156500 |
| 71175 | 134048323 | 134049449 | 101928 |
| 71176 | 134082080 | 134083826 | 236091 |
| 71177 | 134185709 | 134186818 | 203950 |
| 71178 | 134458303 | 134458902 | 186376 |
| 71179 | 134477134 | 134478803 | 173275, 186376 |
| 71180 | 134523229 | 134524774 | |
| 71181 | 135055244 | 135056522 | 169446 |
| 71182 | 135241712 | 135242479 | 022267 |
| 71183 | 135257731 | 135259528 | 022267 |
| 71184 | 135266758 | 135268599 | 022267 |
| 71185 | 135271346 | 135271957 | 022267 |
| 71186 | 135272690 | 135273944 | 022267 |
| 71187 | 135332578 | 135333866 | 129680 |
| 71188 | 135341675 | 135342709 | |
| 71189 | 135578472 | 135579939 | 102241 |
| 71190 | 135792380 | 135793510 | 129675 |
| 71191 | 135896827 | 135897476 | |
| 71192 | 136007174 | 136008278 | 234062 |
| 71193 | 136122257 | 136122621 | |
| 71194 | 138021257 | 138021596 | 129682 |
| 71195 | 138177081 | 138177677 | 129682 |
| 71196 | 138276392 | 138278019 | 129682 |
| 71197 | 138282099 | 138283475 | 129682 |
| 71198 | 138369187 | 138370078 | |
| 71199 | 138612437 | 138615535 | 101981 |
| 71200 | 138905684 | 138906679 | 101974 |
| 71201 | 139013159 | 139016838 | 101974 |
| 71202 | 142082505 | 142083599 | |
| 71203 | 142086665 | 142087986 | |
| 71204 | 146262073 | 146263106 | |
| 71205 | 146957072 | 146958010 | |
| 71206 | 146966234 | 146967035 | |
| 71207 | 146971868 | 146974025 | |
| 71208 | 146992290 | 146994617 | 102081, 268066 |
| 71209 | 147445082 | 147446055 | |
| 71210 | 148586222 | 148587136 | 241489, 010404 |
| 71211 | 148603981 | 148605333 | 238039, 241489, 010404 |
| 71212 | 148621893 | 148623171 | 197620, 241769 |
| 71213 | 148710134 | 148710913 | 155984 |
| 71214 | 148712489 | 148713498 | 155984 |
| 71215 | 149105920 | 149107184 | 235703, 197021 |
| 71216 | 149203225 | 149206126 | 235703 |
| 71217 | 149251395 | 149253989 | 235703 |
| 71218 | 149368184 | 149370223 | 235703 |
| 71219 | 149437763 | 149438233 | |
| 71220 | 149461733 | 149462355 | |
| 71221 | 149492527 | 149492937 | |
| 71222 | 149530141 | 149533479 | 013619 |
| 71223 | 149715374 | 149717503 | |
| 71224 | 149736478 | 149738048 | 171100 |
| 71225 | 149738879 | 149739395 | 171100 |
| 71226 | 149860830 | 149863225 | 063601 |
| 71227 | 149883564 | 149884149 | 063601 |
| 71228 | 150017237 | 150017901 | 102181 |
| 71229 | 150066406 | 150068355 | 102181 |
| 71230 | 150134485 | 150135598 | |
| 71231 | 150151609 | 150152630 | 029993 |
| 71232 | 150564324 | 150566484 | 160131 |
| 71233 | 151139047 | 151139756 | 102287 |
| 71234 | 151150153 | 151151167 | |
| 71235 | 151999227 | 152000762 | 147383, 147400 |
| 71236 | 152109893 | 152110797 | 147394 |
| 71237 | 152599416 | 152600648 | 063587 |
| 71238 | 152888027 | 152889105 | |
| 71239 | 152889582 | 152891892 | |
| 71240 | 152900140 | 152900776 | |
| 71241 | 152988238 | 152990998 | 101986, 185825 |
| 71242 | 152991170 | 152993207 | 101986 |
| 71243 | 153058562 | 153061113 | 180879, 067829 |
| 71244 | 153192716 | 153194081 | 089820 |
| 71245 | 153235598 | 153239144 | 177854, 172534 |
| 71246 | 153284305 | 153286374 | 211524, 184216 |
| 71247 | 153305290 | 153306294 | 169057 |
| 71248 | 153322048 | 153322899 | 169057 |
| 71249 | 153362234 | 153363508 | 169057 |
| 71250 | 153587117 | 153587606 | 196924 |
| 71251 | 153596572 | 153598952 | 196924 |
| 71252 | 153599594 | 153600347 | 196924 |
| 71253 | 153606154 | 153608913 | 102119 |
| 71254 | 153625175 | 153628584 | 147403 |
| 71255 | 153656782 | 153657881 | 071553, 197180 |
| 71256 | 153664990 | 153666301 | 203879 |
| 71257 | 153672242 | 153673648 | 071859 |
| 71258 | 153706541 | 153707910 | 196976 |
| 71259 | 153714085 | 153715191 | 102178 |
| 71260 | 153718138 | 153719591 | 126903 |
| 71261 | 153743501 | 153745581 | 071889 |
| 71262 | 153762780 | 153763625 | 160211 |
| 71263 | 153768622 | 153772015 | 073009, 160211 |
| 71264 | 153774808 | 153776489 | 160211, 073009 |
| 71265 | 153958545 | 153959377 | 160219 |
| 71266 | 153990511 | 153992387 | 130826 |
| 71267 | 154021261 | 154021903 | 130830 |
| 71268 | 154026361 | 154029350 | 130830 |
| 71269 | 154032824 | 154034306 | 130830 |
| 71270 | 154216172 | 154216749 | 185010 |
| 71271 | 154254244 | 154256341 | 165775, 185010 |
| 71272 | 154282524 | 154282963 | 165775 |
| 71273 | 154293429 | 154294209 | 214827, 182712 |
| 71274 | 154296375 | 154297004 | 214827, 182712 |
| 71275 | 154298355 | 154300593 | 185515, 182712, 214827 |
| 71276 | 154444083 | 154445697 | 155959 |
| 71277 | 154841523 | 154843055 | 185973 |

The locations of ODSCs on Chromosome Y are shown in Table 66.

TABLE 66
ODSC Locations on Chromosome Y

| SEQ ID NO. | Start | End | Overlapping Gene(s) |
|---|---|---|---|
| 71278 | 10036363 | 10036795 | 225840 |
| 71279 | 21154432 | 21155381 | 185275, 176728 |

The locations of ODSCs on the mitochondrial chromosome are shown in Table 67.

TABLE 67
ODSC Locations on the Mitochondrial Chromosome

| SEQ ID NO. | Start | End | Overlapping Gene(s) |
|---|---|---|---|
| 71280 | 2 | 4081 | 211459, 198888, 210077, |
| | | | 209082, 210082, 210049 |
| 71281 | 9228 | 16571 | 210191, 210196, 210195, |
| | | | 210194, 198886, 198840, |
| | | | 198727, 210174, 210176, |
| | | | 198786, 212907, 210184, |
| | | | 210164, 198695, 198938 |

Further, results show that about 90% of H3K27ac sites have a signaling protein bound to it. 4,279 sites having a threshold of at least 2 signaling proteins that did not also have at least 2 master transcription factors were observed. Additionally, 49,805 sites were identified having H3K27ac or BRD4 or p300 or at least 2 master transcription factors but not at least 2 signaling proteins. These results provide evidence that not all enhancers are part of an ODSC.

### Example 12. Hepatocyte Gene Expression in Insulated Neighborhoods (IN)

RNA-seq analysis (FPKM<1 and q-value≤0.05 and log 2fc≥1) of hepatocytes stimulated with the compounds of Table 68 was used to classify genes into 3 categories: 1) 10,164 non-expressed genes; 2) 5,904 static genes (expressed and unchanged by treatments), and 3) 7,790 dynamic (wide range of results from only a few compounds).

TABLE 68
Compounds for stimulation of hepatocytes

| ID | Compound Name | Target | Pathway | Action |
|---|---|---|---|---|
| 1 | Simvastatin | HMG-CoA reductase | Metabolic | Inhibitor |
| 2 | Adapin (doxepin) | H1 histamine, α-adrenoreceptors | Histamine receptor signaling | Antagonist |
| 4 | Danazol | ER, AR, Progesteron receptor | Estrogen signaling | Agonist |
| 5 | Nefazodone | HTR2A | Calcium signaling | Antagonist |
| 6 | Rosiglitazone maleate | PPARg | PPAR signaling | Agonist |
| 7 | Sulpiride | D2 dopamine | cAMP signaling | Antagonist |
| 8 | Captopril | MMP2 | Estrogen signaling | Inhibitor |
| 9 | atenolol | ADRB1 | Adrenergic signaling | Antagonist |
| 10 | Ranitidine | H2 histamine receptor | Histamine receptor signaling | Antagonist |
| 11 | Metformin | AMPK | Insulin & AMPK signaling | Activator |
| 12 | imatinib | RTK, Bcr-Abl | PDGFR, ABL signaling | Inhibitor |
| 13 | Papaverine | phosphodiesterase | AMPK signaling | Inhibitor |
| 14 | Amiodarone | Adrenergic receptor β, CYP | Adrenergic signaling | Antagonist |
| 15 | Nitrofurantoin | pyruvate-flavodoxin oxidoreductase | antibiotic | Activator |
| 16 | prednisone | GR | GR signaling | Agonist |
| 17 | Penicillamine(D-) | copper | copper chelation | Chelator |
| 18 | Disopyramide | SCN5A | Adrenergic signaling | Inhibitor |
| 19 | Rifampicin | PXR | PXR | Inhibitor |
| 20 | Benzbromarone | xanthine oxidase, CYP2C9 | uric acid formation | Inhibitor |
| 21 | isoniazid | CYP2C19, CYP3A4 | unknown | Inhibitor |
| 22 | Acetaminophen (paracetamol) | COX1/2 | COX | Inhibitor |
| 23 | Ritonavir | CYP3A4, Pol polyprotein | HIV Transcription | Inhibitor |
| 24 | SGI-1776 | PIM | JAK/STAT signaling | Inhibitor |
| 25 | Valproate | HDAC9, glucuronyl transferase, epoxide | unknown | Inhibitor |
| | | hydrolase | | |
| 26 | Ibuprofen | COX, PTGS2 | COX | Inhibitor |
| 27 | Propylthiouracil | thyroperoxidase | Thyroid hormone synthesis | Inhibitor |
| 28 | rapamycin | mTOR | mTOR signaling | Inhibitor |
| 29 | BIO | GSK-3 | WNT, TGF beta signaling | Inhibitor |
| 30 | ATRA | RXRb, RXRg, RARg | RAR signaling | Agonist |
| 31 | Xav939 | tankyrase | WNT & PARP pathway | Inhibitor |
| 32 | bms189453 | RARB | Nuclear Receptor transcription | Agonist |
| 33 | dorsomorphin | ALK | TGF beta signaling | Inhibitor |
| 34 | BMP2 | BMPR1A | TGF beta signaling | Agonist |
| 35 | BMS777607 | Met | Ras signaling | Inhibitor |
| 36 | bms833923 | SMO | Hedgehog signaling | Antagonist |
| 37 | dmPGE2 | EPR, PGDH | EP receptor signaling | Agonist |
| 38 | MK-0752 | y-secretase | NOTCH signaling | Inhibitor |
| 39 | N-Acetylpurinomycin | SnoN, SKI, SKIL | TGF beta signaling | Modulator |
| 40 | LY 364947 | TGF-β RI, TGFR-I, TβR-I, | TGF beta signaling | Inhibitor |
| | | ALK-5 | | |
| 41 | Enzastaurin | PKC | Epigenetics; TGF-beta/Smad | Inhibitor |
| 42 | DMXAA | Unclear | Tumor necrosis | Inhibitor |
| 43 | BSI-201 | PARP | Cell Cycle/DNA Damage; Epigenetics | Inhibitor |
| 44 | Darapladib | Phospholipase | Others | Inhibitor |
| 45 | Selumetinib | MEK | MAPK/ERK Pathway | Inhibitor |
| 46 | Peramivir (trihydrate) | Influenza Virus | Anti-infection | Inhibitor |
| 47 | Palifosfamide | DNA alkylator/crosslinker | Cell Cycle/DNA Damage | |
| 48 | Evacetrapib | CETP | Others | Inhibitor |
| 49 | Cediranib | VEGFR | Protein Tyrosine Kinase/RTK | Inhibitor |
| 50 | R788 (fostamatinib, disodium | Syk | Protein Tyrosine Kinase/RTK | Inhibitor |
| | hexahydrate) | | | |
| 51 | Torcetrapib | CETP | Others | Inhibitor |
| 52 | Tivozanib | VEGFR | Protein Tyrosine Kinase/RTK | Inhibitor |
| 53 | 17-AAG (Tanespimycin) | HSP | Cell Cycle/DNA Damage Metabolic | Inhibitor |
| | | | Enzyme/Protease | |
| 54 | Zibotentan | Endothelin Receptor | GPCR/G protein | Antagonist |
| 55 | Semagacestat | γ-secretase | Neuronal Signaling Stem Cells/Wnt | Inhibitor |
| 56 | Dalcetrapib | CETP | Others | Inhibitor |
| 57 | Latrepirdine (dihydrochloride) | AMPK | Epigenetics; PI3K/Akt/mTOR | Activator |
| 58 | CMX001 (Brincidofovir) | CMV | Anti-infection | NA |
| 59 | Vicriviroc (maleate) | CCR | GPCR/G protein; | Antagonist |
| | | | Immunology/Inflammation | |
| 60 | Temsirolimus | mTOR | PI3K/Akt/mTOR | Inhibitor |
| 61 | Preladenant | Adenosine Receptor | GPCR/G protein | Antagonist |
| 62 | EVP-6124 (hydrochloride) | nAChR | Membrane Transporter/Ion Channel | Activator |
| | (encenidine) | | | |
| 63 | Bitopertin | GlyT1 | Membrane Transporter/Ion Channel | Inhibitor |
| 64 | Latrepirdine | AMPK | Epigenetics; PI3K/Akt/mTOR | Inhibitor |
| 65 | Vanexerine (dihydrochleride) | Depamine Reuptake Inhibitor | Neuronal Signaling | Inhibitor |
| 66 | CO-1686 (Rociletinib) | EGFR | JAK/STAT Signaling Protein Tyrosine | Inhibitor |
| | | | Kinase/RTK | |
| 67 | Laropiprant (tredaptive) | Prostaglandin Recepter | GPCR/G protein | Antagonist |
| 68 | Bardoxolone | Keap1-Nrf2 | NF-κB | Activator |
| 69 | VX-661 (tezacaptor) | CFTR | Membrane transporter/ion channel | Corrector |
| 70 | INNO-206 (aldoxorubicin) | Topoisomerase | Cell Cycle/DNA Damage | NA |
| 71 | LY404039 (pomaglumetad | mGluR | GPCR/G protein | Inhibitor |
| | methionil (mGlu2/3)) | | | |
| 72 | Perifosine (KRX-0401) | AKT | PI3K/AKT | Inhibitor |
| 73 | Cabozantinib (XL184, BMS- | VEGFR2, MET, Ret, Kit, Flt-1/3/4, | MET | Inhibitor |
| | 907351) | Tie2, and AXL | | |
| 74 | Dacomitinib (PF299804, PF299) | EGFR, ErbB2, ErbB4 | AKT/ERK, HER | Inhibitor |
| 75 | Pacritinib (SB1518) | FLT3, JAK2, TYK2, JAK3, JAK1 | JAK-STAT | Inhibitor |
| 76 | TH-302 (Evofosfamide) | hypoxic regions | Unclear | NA |
| 77 | α-PHP | Unclear | Unclear | NA |
| 78 | LY 2140023 (Pomaglumetad | mGlu₂ & mGlu₃ | Gαi/o protein-dependent | Activator |
| | methionil-LY404039) | | | |
| 79 | TP-434 (Eravacycline) | Antibiotic resistance mechanisms | Tetracycline-specific efflux | Inhibitor |
| 80 | TC-5214 (S-(+)-MecaMylaMine | Nicotinic acetylcholine receptors | Base excision repair and homologous | Antagonist |
| | Hydrochloride) | | recombination repair | |
| 81 | Rolofylline (KW-3902) | A1 adenosine receptor | Unclear | Antagonist |
| 82 | Amigal (Deoxygalactonojirimycin | a-galactosidase | Stress signaling | Inhibitor |
| | hydrochloride) | | | |
| 83 | NOV-002 (oxidized L- | gamma-glutamyl-transpeptidase (GGT) | Glutathione pathway | NA |
| | Glutathione) | | | |
| 84 | bms-986094 (inx-189) | NS5B | Unclear | Inhibitor |
| 85 | TC-5214 (R-Mecamylamine | Nicotinic receptors | Base excision repair and homologous | Antagonist |
| | hydrochloride) | | recombination repair | |
| 86 | Ganaxolone | GBAA receptors | Unclear | Modulator |
| 87 | Irinotecan Hydrochloride | DNA Topol | Unclear | Inhibitor |
| | Trihydrate | | | |
| 88 | TFP | D2R, Calmpdulin | Calmpdulin | Inhibitor |
| 89 | Perphenazine | D2R, Calmpdulin | Calmpdulin | Inhibitor |
| 90 | A3-HCl | CKI, CKII, PKC, PKA | WNT, Hedgehog, PKC, PKA | Inhibitor |
| 91 | FICZ | Aryl hydrocarbon receptor | Aryl hydrocarbon receptor | Agonist |
| 92 | Pifithrin-α | p53 | p53 | Inhibitor |
| 93 | Deferoxamine mesylate | HIF | Hypoxia activated | Inhibitor |
| 94 | Insulin | InsR | IGF-1R/InsR | Activator |
| 95 | Phorbol 12,13-dibutyrate | PKC | PKC | Activator |
| 96 | RU 28318 | MR | Mineralcorticoid receptor | Antagonist |
| 97 | Bryostatin1 | PKC | PKC | Activator |
| 98 | DY 268 | FXR | FXR | Antagonist |
| 99 | GW 7647 | PPARα | PPAR | Agonist |
| 100 | CI-4AS-1 | AR | Androgen receptor | Agonist |
| 101 | T0901317 | LXR | LXR | Agonist |
| 102 | BMP2 | BMPR1A | TGF-B | Activator |
| 103 | 22S-Hydroxycholesterol | LXR | LXR | Inhibitor |
| 104 | CALP1 | Calmodulin | Calmodulin | Activator |
| 105 | CALP3 | Calmodulin | Calmodulin | Activator |
| 106 | Forskolin | Adenylyl cyclase | cAMP related | Activator |
| 107 | Dexamethasene | GR | Glucocorticoid receptor | Activator |
| 108 | IFN-y | IFNGR1/IFNGR2 | JAK/STAT | Activator |
| 109 | TGF-b | TGF-beta Receptor | TGF-B | Activator |
| 110 | TNF-a | TNF-R1/TNF-R2 | NF-kB, MAPK, Apoptosis | Activator |
| 111 | PDGF | Pan-PDGFR | PDGFR | Activator |
| 112 | IGF-1 | IGF-1R | IGF-1R/InsR | Activator |
| 113 | FGF | FGFR | FGFR | Activator |
| 114 | EGF | Pan-ErbB | EGFR | Activator |
| 115 | HGF/SF | c-Met | c-MET | Activator |
| 116 | TCS 359 | FLT3 | Protein Tyrosine Kinase/RTK | Inhibitor |
| 117 | Cobalt chloride | HIF1 | Hypoxia activated | Inducer |
| 118 | CH223191 | AhR | Aryl hydrocarbon receptor | Antagonist |
| 119 | Echinomycin | HIF | Hypoxia activated | Inhibitor |
| 120 | PAF C-16 | MEK | MAPK | Activator |
| 121 | Bexarotene | RXR | RXR | Agonist |
| 122 | CD 2665 | RAR | RAR | Antagonist |
| 123 | Pifithrin-μ | p53 | p53 | Inhibitor |
| 124 | EB1089 | VDR | Vitamin D Receptor | Agonist |
| 125 | BMP4 | TGF-beta | TGF-B | Activator |
| 126 | IWP-2 | Wnt | WNT | Inhibitor |
| 127 | RITA (NSC 652287) | p53 | p53 | Inhibitor |
| 128 | Calcitriol | VDR | Vitamin D Receptor | Agonist |
| 129 | ACEA | CB1 | Cannabinoid receptor | Agonist |
| 130 | Rimonabant | CB1 | Cannabinoid receptor | Antagonist |
| 131 | Otenabant | CB1 | Cannabinoid receptor | Antagonist |
| 132 | DLPC | LRH-1/NR5A2 | LHR-1 | Agonist |
| 133 | LRH-1 antagonist | LRH-1/NR5A3 | LHR-1 | Antagonist |
| 134 | Wnt3a | FRIZZLED | WNT | Activator |
| 135 | Activin | TGF-beta | TGF-B | Activator |
| 136 | Nodal | TGF-beta | TGF-B | Activator |
| 137 | Anti mullerian hormone | TGF-beta | TGF-B | Activator |
| 138 | GDF2 (BMP9) | TGF-beta | TGF-B | Activator |
| 139 | GDF10 (BMP3b) | TGF-beta | TGF-B | Activator |
| 140 | Oxoglaucine | PI3K/Akt | PI3K/AKT | Activator |
| 141 | BMS 195614 | RAR | RAR | Antagonist |
| 142 | LDN193189 | ALK2/3 | TGF-B | Inhibitor |
| 143 | Varenicline Tartrate | AchR | Acetylcholine receptor | Agonist |
| 144 | Histamine | Histamine receptor | Histamine receptor | Activator |
| 145 | Chloroquine phosphate | ATM/ATR | ATM/ATR | Activator |
| 146 | LJI308 | RSK1/2/3 | S6K | Inhibitor |
| 147 | GSK621 | AMPK | AMPK | Activator |
| 148 | STA-21 | STAT3 | JAK/STAT | Inhibitor |
| 149 | SMI-4a | Pim1 | PIM | Inhibitor |
| 150 | AMG 337 | c-Met | c-MET | Inhibitor |
| 151 | Wnt agonist 1 | Wnt | WNT | Activator |
| 152 | PRI-724 | Wnt | WNT | Inhibitor |
| 153 | ABT-263 | Pan-Bcl-2 | BCL2 | Inhibitor |
| 154 | Axitinib | Pan-VEGFR | VEGFR | Inhibitor |
| 155 | Afatinib | Pan-ErbB | EGFR | Inhibitor |
| 156 | Bosutinib | Src | Src | Inhibitor |
| 157 | Dasatinib | Bcr-Abl | ABL | Inhibitor |
| 158 | Masitinib | c-Kit | c-KIT | Inhibitor |
| 159 | Crizotinib | c-Met | c-MET | Inhibitor |
| 160 | PHA-665752 | c-Met | c-MET | Inhibitor |
| 161 | GSK1904529A | IGF-1R/InsR | IGF-1R/InsR | Inhibitor |
| 162 | GDC-0879 | Raf | MAPK | Inhibitor |
| 163 | LY294002 | Pan-PI3K | PI3K/AKT | Inhibitor |
| 164 | OSU-03012 | PDK-1 | PDK-1 | Inhibitor |
| 165 | JNJ-38877605 | c-Met | c-MET | Inhibitor |
| 166 | BMS-754807 | IGF-1R/InsR | IGF-1R/InsR | Inhibitor |
| 167 | TGX-221 | p110b | PI3K/AKT | Inhibitor |
| 168 | Regorafenib | Pan-VEGFR | VEGFR | Inhibitor |
| 169 | Thalidomide | AR | NF-kB | Antagonist |
| 170 | Amuvatinib | PDGFRA | PDGFR | Inhibitor |
| 171 | Etomidate | GABA | GABAergic receptor | Inhibitor |
| 172 | Glimepiride | Potassium channel | Potassium channel | Inhibitor |
| 173 | Omeprazole | Proton pump | Proton pump | Agonist |
| 174 | Tipifarnib | Ras | RAS | Inhibitor |
| 175 | SP600125 | Jnk | MAPK | Inhibitor |
| 176 | Quizartinib | FLT3 | FLT3 | Inhibitor |
| 177 | CP-673451 | Pan-PDGFR | PDGFR | Inhibitor |
| 178 | Pomalidomide | TNF-a | NF-kB | Inhibitor |
| 179 | KU-60019 | ATM kinase | DNA Damage | Inhibitor |
| 180 | BIRB 796 | p38 | MAPK | Inhibitor |
| 181 | RO4929097 | Gamma-secretase | NOTCH | Inhibitor |
| 182 | Hydrocortisone | GR | Glucocorticoid receptor | Agonist |
| 183 | AICAR | AMPK | AMPK | Activator |
| 184 | Amlodipine Besylate | Calcium channel | Calcium channel | Inhibitor |
| 185 | DPH | Bcr-Abl | ABL | Activator |
| 186 | Taladegib | Hedgehog/Smoothened | Hedgehog/Smoothened | Inhibitor |
| 187 | AZD1480 | JAK2 | JAK/STAT | Inhibitor |
| 188 | AST-1306 | Pan-ErbB | EGFR | Inhibitor |
| 189 | AZD8931 | Pan-ErbB | EGFR | Inhibitor |
| 190 | Momelotinib | Pan-Jak | JAK/STAT | Inhibitor |
| 191 | Cryptotanshinone | STAT3 | JAK/STAT | Inhibitor |
| 192 | Bethanechol chloride | AchR | Acetylcholine receptor | Activator |
| 193 | Clozapine | 5-HT | 5-HT | Antagonist |
| 194 | Dopamine | Dopamine | Dopamine receptor | Agonist |
| 195 | Phenformin | AMPK | AMPK | Activator |
| 196 | Mifepristone | GR | Glucocorticoid receptor | Antagonist |
| 197 | GW3965 | LXR | LXR | Agonist |
| 198 | WYE-125132 (WYE-132) | mTOR | mTOR | Inhibitor |
| 199 | Crenolanib | Pan-PDGFR | PDGFR | Inhibitor |
| 200 | PF-04691502 | Pan-Akt | PI3K/AKT | Inhibitor |
| 201 | GW4064 | FXR | FXR | Agonist |
| 202 | Sotrastaurin | PKC | PKC | Inhibitor |
| 203 | Ipatasertib | Pan-Akt | PI3K/AKT | Inhibitor |
| 204 | ARN-509 | AR | Androgen receptor | Inhibitor |
| 205 | T0070907 | PPARg | PPAR | Antagonist |
| 206 | GO6983 | PKC | PKC | Inhibitor |
| 207 | Epinephrine | Adrenergic | Adrenergic receptor | Agonist |
| 208 | Eletriptan | 5-HT | 5-HT | Agonist |
| 209 | Trifluoperazine | Dopamine | Dopamine receptor | Inhibitor |
| 210 | Fexofenadine | Histamine | Histamine receptor | Inhibitor |
| 211 | Corticosterone | MR | Mineralcorticoid receptor | Agonist |
| 212 | Tamibarotene | RAR | RAR | Agonist |
| 213 | Leucine | mTOR | mTOR | Activator |
| 214 | Glycopyrrolate | AchR | Acetylcholine receptor | Antagonist |
| 215 | Tiagabine | GABA | GABAergic receptor | Inhibitor |
| 216 | Fluoxymesterone | AR | Androgen receptor | Agonist |
| 217 | Tamsulosin hydrochloride | Adrenergic | Adrenergic receptor | Antagonist |
| 218 | Ceritinib | ALK | ALK | Inhibitor |
| 219 | GSK2334470 | PDK-1 | PDK-1 | Inhibitor |
| 220 | AZD1208 | Pan-PlM | PIM | Inhibitor |
| 221 | CGK733 | ATM/ATR | DNA Damage | Inhibitor |
| 222 | LDN-212854 | Pan-TGFB | TGF-B | Inhibitor |
| 223 | GZD824 Dimesylate | Bcr-Abl | ABL | Inhibitor |
| 224 | AZD2858 | Pan-GSK-3 | GSK-3 | Inhibitor |
| 225 | FRAX597 | PAK | PAK | Inhibitor |
| 226 | SC75741 | NF-kB | NF-kB | Inhibitor |
| 227 | SH-4-54 | Pan-STAT | JAK/STAT | Inhibitor |
| 228 | HS-173 | p110a | PI3K/AKT | Inhibitor |
| 229 | K02288 | Pan-TGFB | TGF-B | Inhibitor |
| 230 | EW-7197 | Pan-TGFB | TGF-B | Inhibitor |
| 231 | Decernotinib | Pan-Jak | JAK/STAT | Inhibitor |
| 232 | MI-773 | p53 | p53 | Inhibitor |
| 233 | PND-1186 | FAK | FAK | Activator |
| 234 | Kartogenin | SMAD4/5 | TGF-B | Activator |
| 235 | Picropodophyllin | IGF-1R | IGF-1R/InsR | Inhibitor |
| 236 | AZD6738 | ATR | ATM/ATR | Inhibitor |
| 237 | Smoothened Agonist | Hedgehog/Smoothened | Hedgehog/Smoothened | Agonist |
| 238 | Erlotinib | EGFR/ErbB1 | EGFR | Inhibitor |
| 239 | MHY1485 | mTOR | mTOR | Activator |
| 240 | SC79 | Pan-Akt | PI3K/AKT | Activator |
| 241 | meBIO | AhR | Aryl hydrocarbon receptpr | Agonist |
| 242 | Huperzine | AchE | Acetylcholine receptor | Inhibitor |
| 243 | BGJ398 | Pan-FGFR | FGFR | Inhibitor |
| 244 | Netarsudil | ROCK | ROCK | Inhibitor |
| 245 | Acetycholine | AchR | Acetylcholine receptor | Agonist |
| 246 | Purmprphamine | Hedgehog/Smoothened | Hedgehog/Smoothened | Agonist |
| 247 | LY2584702 | p70 S6K | S6K | Inhibitor |
| 248 | Dorsomorphin | AMPK | AMPK | Inhibitor |
| 249 | Glasdegib (PF-04449913) | Hedgehog/Smoothened | Hedgehog/Smoothened | Inhibitor |
| 250 | LDN193189 | Pan-TGFB | TGF-B | Inhibitor |
| 251 | Oligpmycin A | ATPase | ATP channel | Inhibitor |
| 252 | BAY 87-2243 | HIF1 | Hypoxia activated | Inhibitor |
| 253 | SIS3 | SMAD3 | TGF-B | Inhibitor |
| 254 | BDA-366 | Bcl-2 | BCL2 | Antagonist |
| 255 | XMU-MP-1 | MST1/2 | Hippo | Inhibitor |
| 256 | Semaxinib | Pan-VEGFR | VEGFR | Inhibitor |
| 257 | BAM7 | Bcl-2 | BCL2 | Activator |
| 258 | GDC-0994 | Erk | MAPK | Inhibitor |
| 259 | SKL2001 | Wnt | WNT | Agonist |
| 260 | Merestinib | c-Met | c-MET | Inhibitor |
| 261 | APS-2-79 | MEK | MAPK | Antagonist |
| 262 | NSC228155 | Pan-ErbB | EGFR | Activator |
| 263 | 740 Y-P | Pan-PI3K | PI3K/AKT | Activator |
| 264 | b-Estradipl | ER | ER | Activator |
| 265 | Glucose | GLUTs | metabolic/glycolysis | Activator |
| 266 | Transferrin | Transferrin Receptor | Iron transport | Activator |
| 267 | AM 580 | RAR | RAR | Activator |

AADAC, ABLIM3, ACAA2, ACADVL, ACAT2, ACKR2, ACMSD, ACSL1, ACSL5, ACSM2A, ACSS2, ACTA1, ACTA2, ADM, ADRB2, AFAP1L1, AGAP1-IT1, AGPAT2, AGRN, AGTR1, AHSG, AKAP12, AKR1C6P, AKR1C8P, ALAS1, ALDH1L1, ALDH8A1, ALPK2, AMACR, ANGPTL4, ANKRD1, ANKRD29, ANO1, ANP32AP1, ANXA1, ANXA3, APOA4, ARG1, ARHGEF26, ARL14, ARL2, ARL4A, ARL4D, ARRDC3, ARTS, ATP2B4, ATP5EP2, ATP6V0D2, B4GALNT1, BAG3, BCAS1, BCAT2, BCL2A1, BIRC3, BLOC1S1-RDH5, BMF, BMP2, BNIP3, BRF2, C10orf32-ASMT, C10orf35, C18orf32, C19orf73, C19orf80, C1orf162, C1orf220, C1orf226, C1orf53, C2orf54, C2orf82, C4orf19, C8orf4, CA12, CA2, CACHD1, CAMK2G, CAMKMT, CAPN13, CAT, CBLB, CCBL1, CCL2, CCL26, CCL5, CD109, CDC6, CEBPD, CES4A, CHAD, CHKB-AS1, CHST13, CHST9, CIDEC, CITED2, CLDN14, CLDN2, CLDN4, CLDND2, CLRN3, COL1A1, COX7A1, CPA4, CPS1, CPT1A, CPT2, CRACR2B, CREB3L3, CRIP3, CRYAB, CRYM, CSDC2, CTDSP1, CUX2, CX3CL1, CXCL1, CXCL10, CXCL11, CXCL2, CXCL3, CXCL8, CYB561A3, CYP1A1, CYP1A2, CYP1B1, CYP21A2, CYP2C19, CYP2C9, CYP3A4, CYP3A43, CYP3A5, CYP3A7, CYP8B1, CYTH1, DEDD2, DHRS2, DNAJB1, DNAJC12, DNAJC22, DRAIC, DUOX2, DUOXA2, EAF2, ECH1, ECM2, EDN1, EFNA1, ELOVL2, EMP2, ENO2, ENO3, ENTPD7, EPB41L1, EPSTI1, ERICH5, ERRFI1, ETFDH, ETNK2, ETNPPL, F2RL2, FABP1, FABP3, FADS1, FADS2, FAHD2B, FAM134B, FAM13A, FAM151A, FAM162A, FAM198A, FAM219A, FAM229A, FAM25A, FAM26F, FAM83D, FASN, FCAMR, FDPS, FGF2, FGF21, FGFR3, FHIT, FKBP1AP1, FKBP5, FLJ44635, FLNA, FLNC, FLRT3, FLVCR2, FMO1, FOSL1, FSTL3, FXYD2, G0S2, G6PC, GADD45B, GAL3ST1, GBP4, GDF15, GEM, GHR, GLA, GLYCTK-AS1, HAO2, HBEGF, HGD, HHAT, HIST1H2BG, HIST1H4E, HIST3H2A, HKDC1, HLA-G, HMGCR, HMGCS1, HMGCS2, HMOX1, HOGA1, HPGD, HRCT1, HS1BP3-IT1, HSD17B7, HSDL2, HSPA1A, HSPA1B, HSPA5, HSPA6, HSPB1, HSPB8, HSPH1, HYAL3, IPI1, IER3, IFI27, IFI35, IFIT1, IFIT2, IFITM1, IFT27, IGFBP1, IGFBP3, IGFBP6, IGSF23, IL17RB, IL18, IL23A, IL36B, INHBA, INHBE, INSIG1, IQCD, IRF5, ISM1-AS1, ITGA3, ITGA5, ITGA7, ITGB6, IVL, KANK1, KCNJ8, KLF15, KLF2, KLF9, KRT7, KRT80, KRT81, KSR2, LAMA3, LAMB1, LAMB3, LBH, LDHA, LDLRAD4, LECT2, LGALS1, LGALS4, LGALS9, LIF, LINC00152, LINC00261, LINC00704, LINC00880, LINC01023, LINC01151, LINC01214, LINC01336, LINC01554, LINC01587, LMCD1, LOC100133985, LOC100507002, LOC101927686, LOC101928505, LOC101929427, LOC101929613, LOC102467080, LOC102724450, LOC153910, LOC344887, LOC728040, LOXL4, LPAL2, LPIN2, LRCOL1, LRRN2, LTBP1, LUCAT1, LYPD1, LYPLAL1-AS1, LYZ, MAFB, MAFF, MANF, MAGA, MAP3K8, MATN2, MCAM, MCM7, METRN, MGC12916, MLLT11, MMP12, MMP24, MMP3, MOGAT2, MPPED1, MPV17L, MSC, MSHS-SAPCD1, MSMO1, MTMR11, MTRNR2L1, MTRNR2L10, MTRNR2L3, MTRNR2L6, MTRNR2L9, MUC3A, MVD, MXRA8, MYH16, MYL9, MYOF, MYOM1, NAP1L2, NBPF13P, NCF2, NCR3LG1, NDRG1, NEURL3, NFASC, NFATC4, NMB, NPAS2, NPHP3-ACAD11, NPPB, NR6A1, NRARP, NRAV, NRBP2, NTN1, NUAK2, NUCB2, OAS1, OAS2, OAS3, OASL, OSGIN1, OTUD3, OXTR, P4HA1, PALMD, PAM, PAPLN, PBXIP1, PCK1, PCSK9, PDGFB, PDGFRB, PDIA4, PDK1, PDK4, PDLIM7, PDZK1, PELI3, PER1, PFDN4, PFKFB4, PGM2, PHLDA1, PID1, PIGR, PLCXD1, PLIN2, PLIN4, PLK2, PLOD1, PNPLA5, POR, PPAP2B, PPL, PPM1L, PPP1R3C, PRG4, PRR15, PRSS23, PSD3, PSORS1C3, PTTG1, PWAR5, PYCARD, QPRT, RAB17, RAPGEF4, RARB, RASD1, RBP5, RCL1, RGCC, RGS2, RHCG, RHOB, RHOF, RMRP, RN7SK, RN7SL1, RN7SL2, RNA5S9, RND1, RNF139-AS1, RNU12, RNU2-2P, RNU4-1, RNU4-2, RNUSA-1, RNUSB-1, RNUSE-1, RNVU1-15, RORA, RPL17-C18orf32, RPL18A, RPL23P8, RPPH1, RPS15, RPSAP58, RRAD, RSAD2, RTP3, S100A2, S100A3, S100A6, SAA3P, SALL1, SCARA3, SCPEP1, SCUBE1, SDCBP2, SDS, SEC14L4, SELM, SEMA7A, SERF2-C150RF63, SERPINA12, SERPINB9, SERPINB9P1, SERPINE2, SESN3, SFN, SFRP4, SGK2, SKP1P2, SLC16A13, SLC16A3, SLC17A2, SLC1A2, SLC22A25, SLC25A20, SLC25A30, SLC25A42, SLC25A47, SLC2A2, SLC37A4, SLC38A2, SLC51B, SLC6A1, SLC6A6, SLC7A11, SLC7A5, SLCO2B1, SMAP2, SMIM1, SMN1, SMN2, SMOC1, SNAI2, SNCG, SNHG9, SNX29, SOCS2, SORBS1, SORBS2, SORL1, SOX9, SPAG4, SPNS2, SPOCD1, SPP1, SPRR1A, SPRR3, SPRY1, SPRY2, STAMBPL1, STK39, STOM, STX16-NPEPL1, SUGCT, SULT2A1, SUSD1, SUSD3, SYTL2, SYTL4, SYTL5, TAT, TAT-AS1, TBC1D8, TCP10L, TDO2, TEAD2, TGFB2, TGFBI, TGFBR3, THBS1, THRSP, TIPARP, TMC7, TMCC1, TMEM135, TMEM138, TMEM164, TMEM217, TMEM37, TMEM45A, TMEM97, TMSB4X, TNC, TNFAIP2, TNFAIP3, TNFRSF21, TNFSF11, TNS2, TOX3, TP53INP1, TPRG1-AS1, TRAF5, TREML3P, TRIM31, TRIM55, TRPM8, TSC22D3, TSKU, TSPAN5, TSPAN7, TSPEAR-AS1, TSPEAR-AS2, TTYH3, TUBA1A, TUBA4A, TXNIP, UBE2F-SCLY, UBXN11, UCHL1, UGCG, UNC5CL, UNC93A, USP18, USP2, VEGFB, VIM, VLDLR, VWCE, WDR54, WNT11, XAF1, YPEL2, ZBTB16, ZBTB37, ZC3HAV1L, ZMIZ1-AS1, ZMYND8, ZNF837, ZXDB, A1BG, A1CF, A2M, A4GALT, MAS, AAED1, AAK1, AAMDC, MRS, AARS2, AASDHPPT, AASS, MTF, ABAT, ABCA1, ABCA6, ABCA7, ABCA8, ABCB1, ABCB11, ABCB4, ABCC1, ABCC10, ABCC2, ABCC3, ABCC4, ABCC5, ABCC6, ABCC6P1, ABCC9, ABCD1, ABCD3, ABCE1, ABCF2, ABCG1, ABCC5, ABCG8, ABHD10, ABHD11, ABHD13, ABHD14B, ABHD15, ABHD17A, ABHD17B, ABHD17C, ABHD2, ABHD3, ABHD4, ABHD5, ABHD6, ABHD8, ABI1, ABI2, ABL1, ABL2, ABLIM1, ABLIM2, ABR, ABRACL, ABTB1, ABTB2, ACACA, ACACB, ACAD11, ACAD9, ACADSB, ACAP2, ACAP3, ACBD4, ACBD5, ACBD6, ACID, ACE2, ACER2, ACHE, ACKR3, ACLY, ACOT12, ACOT13, ACOT4, ACOT7, ACOT8, ACOX2, ACOX3, ACSF2, ACSF3, ACSL3, ACSM2B, ACSM3, ACSM5, ACSS1, ACTB, ACTG1, ACTN1, ACTN4, ACTR10, ACTR1A, ACTR2, ACTR3, ACTR5, ACTR6, ACTR8, ACTRT3, ACVR1, ACVR1B, ACVR2A, ADA, ADAM10, ADAM19, ADAM1A, ADAM8, ADAMTS1, ADAMTS10, ADAMTS17, ADAMTS9, ADAMTSL3, ADAMTSL4-AS1, ADAMTSL5, ADARB1, ADAT2, ADAT3, ADCK1, ADCK2, ADCK3, ADCK5, ADCY6, ADCY9, ADD3, ADGRG1, ADGRG6, ADGRL2, ADH1A, ADH1B, ADH1C, ADH4, ADH6, ADHFE1, ADI1, ADIPOR2, ADIRF, ADK, ADM2, ADPGK, ADPRH, ADPRHL2, ADRA1A, ADRA1B, ADSL, ADSS, ADSSL1, AEBP2, AEN, AFAP1, AFF1, AFF3, AFF4, AFG3L1P, AFM, AFMID, AFP, AGAP1, ACAP3, AGBL5, AGER, AGFG1, AGFG2, AGK, AGL, AGMO, AGO1, AGO4, AGPAT3, AGPAT9, AGPS, AGT, AGTPBP1, AGTRAP, AGXT, AGXT2, AHCYL2, AHDC1, AHI1, AHR, AHSA1, AIM1, AIM1L, AIMP2, AK1, AK2, AK4, AK6, AK9, AKAP1, AKAP11, AKAP13, AKAP2, AKAP7, AKAP9, AKNA, AKR1B1, AKR1B10, AKR1B15, AKR1C1, AKR1C2, AKR1C3, AKR1C4, AKR1D1, AKR7A3, AKT1, AKT1S1, AKT3, ALAD, ALCAM, ALDH16A1, ALDH18A1, ALDH1A1, ALDH1B1, ALDH2, ALDH3A1, ALDH3A2, ALDH4A1, ALDH5A1, ALDH6A1, ALDOA, ALDOB, ALG12, ALG14, ALKBH2, ALKBH3, ALKBH6, ALKBH7, ALKBH8, ALMS1, ALOX12-AS1, ALOXE3, ALPK1, ALPK3, ALPL, ALS2, AMBP, AMBRA1, AMDHD2, AMHR2, AMIGO1, AMMECR1, AMN, AMN1, AMOT, AMOTL1, AMOTL2, AMPD2, AMPD3, AMT, AMY2B, AMZ2, ANAPC1, ANAPC11, ANAPC4, ANG, ANGEL1, ANGEL2, ANGPTL3, ANKEF1, ANKFY1, ANKHD1, ANKMY1, ANKMY2, ANKRA2, ANKRD11, ANKRD13A, ANKRD13B, ANKRD13D, ANKRD16, ANKRD17, ANKRD2, ANKRD24, ANKRD26, ANKRD28, ANKRD30BL, ANKRD32, ANKRD33B, ANKRD37, ANKRD39, ANKRD42, ANKRD44, ANKRD50, ANKRD52, ANKRD54, ANKRD9, ANKS1A, ANKS4B, ANKS6, ANKZF1, ANO10, ANO5, ANO6, ANP32A, ANP32E, ANPEP, ANXA10, ANXA13, ANXA2, ANXA2P1, ANXA2P2, ANXA2P3, ANXA8L1, ANXA9, AOC2, AOC3, AOX1, AP1B1, AP1G1, AP1G2, AP1S3, AP2A1, AP2A2, AP3B1, AP3M1, AP3S2, AP4B1, AP4E1, AP5B1, AP5M1, AP5S1, APAF1, APBA1, APBB1, APBB1IP, APBB2, APBB3, APC, APCS, APEX2, APOA1, APOA2, APOA5, APOB, APOBEC3C, APOBEC3G, APOC1, APOC1 P1, APOC2, APOC3, APOC4, APOE, APOF, APOH, APOL1, APOL2, APOL3, APOL6, APOM, APPBP2, APPL2, APRT, AQP3, AQP4, AQP7, AQP7P3, AQP9, AQR, AR, ARAP2, AREL1, ARF3, ARF5, ARF6, ARFGAP3, ARFGEF1, ARFGEF2, ARG2, ARHGAP10, ARHGAP12, ARHGAP18, ARHGAP24, ARHGAP26, ARHGAP27, ARHGAP29, ARHGAP32, ARHGAP35, ARHGAP39, ARHGAP42, ARHGAP5-AS1, ARHGDIA, ARHGDIB, ARHGEF1, ARHGEF10L, ARHGEF11, ARHGEF12, ARHGEF16, ARHGEF17, ARHGEF18, ARHGEF2, ARHGEF28, ARHGEF40, ARHGEF5, ARID1B, ARID2, ARID4A, ARID5A, ARID5B, ARIH1, ARL10, ARL13B, ARL14EP, ARL15, ARL4C, ARL5B, ARL6, ARL6IP1, ARL6IP4, ARL6IP5, ARL6IP6, ARMC1, ARMC5, ARMC7, ARMC8, ARMC9, ARMCX2, ARMCX4, ARMCX5, ARMCX5-GPRASP2, ARMT1, ARNTL, ARNTL2, ARPC1B, ARPP19, ARRDC1, ARRDC2, ARRDC4, ARSA, ARSB, ARSJ, ARSK, ART4, ARVCF, AS3MT, ASAP1, ASAP2, ASAP3, ASB13, ASB16-AS1, ASB9, ASCC1, ASCC3, ASCL1, ASF1A, ASGR2, ASH1L, ASH2L, ASL, ASNS, ASNSD1, ASPA, ASPDH, ASPG, ASPH, ASRGL1, ASTN2, ASUN, ASXL1, ASXL2, ATAD2, ATAD2B, ATAD3A, ATAD3B, ATAT1, ATE1, ATF2, ATF3, ATF4, ATF5, ATF6, ATF6B, ATF7IP, ATF7IP2, ATG10, ATG13, ATG14, ATG16L2, ATG2A, ATG2B, ATG3, ATG4C, ATG4D, ATG7, ATG9A, ATHL1, ATIC, ATL2, ATM, ATMIN, ATN1, ATOH8, ATOX1, ATP10D, ATP11A, ATP11C, ATP13A1, ATP13A2, ATP13A3, ATP1A1, ATP1B1, ATP1B3, ATP2A2, ATP2B1, ATP2B2, ATP2C1, ATP5D, ATP5E, ATP5G3, ATP5H, ATP5I, ATP5L, ATP6V1E1, ATP6V1H, ATP7A, ATP7B, ATP8B1, ATP9A, ATP9B, ATPIF1, ATR, ATRIP, ATRN, ATRX, ATXN1, ATXN10, ATXN2, ATXN7, ATXN7L2, ATXN7L3, ATXN7L3B, AURKA, AURKAIP1, AURKC, AUTS2, AVEN, AVPI1, AXL, AZGP1, B2M, B3GALT6, B3GAT1, B3GAT3, B3GNT3, B3GNT9, B3GNTL1, B4GALT5, B4GALT6, B4GAT1, B9D2, BMT, BACH1, BACH2, BAD, BAG2, BAG5, BAHCC1, BAIAP2, BAIAP2-AS1, BAIAP2L1, BAMBI, BANP, BATF, BATF2, BAZ1A, BAZ1B, BBC3, BBOX1, BBS1, BBS10, BBS2, BBS5, BBS7, BBS9, BBX, BCAM, BCAR1, BCAR3, BCAS2, BCAS3, BCAS4, BCHE, BCKDHB, BCKDK, BCL2, BCL2L1, BCL2L10, BCL2L11, BCL3, BCL6, BCL7A, BCL7B, BCL7C, BCL9, BCL9L, BCO2, BCOR, BCORL1, BCR, BDH1, BDKRB2, BDP1, BEND3, BET1, BEX2, BEX5, BHLHA15, BHLHB9, BHLHE40, BHLHE41, BHMT, BICC1, BICD1, BIK, BIN1, BIRC6, BIVM, BIVM-ERCC5, BLCAP, BLMH, BLNK, BLOC1S1, BLOC1S3, BLOC1S4, BLZF1, BMP1, BMP2K, BMP4, BMPR1A, BMPR2, BMS1, BMS1P20, BNIP1, BNIP3L, BOD1, BOK, BOLA1, BOP1, BPTF, BRAF, BRAT1, BRD3, BRD4, BRD7, BRD8, BRE, BRF1, BRI3, BRI3BP, BRMS1, BRMS1L, BRPF3, BRWD3, BSCL2, BST1, BTAF1, BTBD11, BTBD19, BTBD2, BTBD3, BTBD9, BTD, BTG1, BTG2, BTG3, BTN2A2, BTN3A1, BTN3A2, BTN3A3, BTRC, BZW1, C10orf10, C10orf11, C10orf12, C10orf54, C10orf76, C11orf24, C11orf30, C11orf49, C11orf54, C11orf74, C11orf84, C11orf91, C11orf95, C11orf96, C11orf98, C12orf10, C12orf4, C12orf45, C12orf57, C12orf60, C12orf75, C12orf76, C14orf105, C14orf159, C14orf169, C14orf2, C14orf28, C14orf37, C14orf79, C14orf80, C14orf93, C15orf39, C15orf41, C15orf48, C15orf52, C15orf57, C16orf45, C16orf46, C16orf52, C16orf59, C16orf62, C16orf86, C16orf87, C16orf91, C16orf95, C17orf100, C17orf51, C17orf58, C17orf59, C17orf62, C17orf75, C17orf80, C17orf85, C17orf89, C17orf96, C17orf97, C18orf21, C18orf25, C19orf12, C19orf24, C19orf48, C19orf54, C19orf60, C19orf68, C19orf70, C1GALT1, C1QBP, C1QTNF3, C1QTNF3-AMACR, C1QTNF6, C1R, C1RL-AS1, C1S, C1orf105, C1orf112, C1orf115, C1orf122, C1orf159, C1orf168, C1orf228, C1orf35, C1orf54, C2, C20orf194, C20orf27, C20orf96, C21orf2, C21orf59, C21orf91, C22orf46, C2CD2, C2CD2L, C2CD3, C2CD5, C2orf42, C2orf72, C2orf74, C3, C3P1, C3orf18, C3orf33, C3orf52, C3orf58, C3orf62, C4A, C4BPA, C4BPB, C4orf27, C4orf46, C4orf48, C5, C5orf24, C5orf28, C5orf42, C5orf46, C5orf51, C5orf56, C5orf63, C6, C6orf106, C6orf136, C6orf141, C6orf203, C6orf226, C6orf47, C6orf48, C6orf52, C6orf62, C7orf25, C7orf26, C7orf43, C7orf49, C7orf50, C8A, C8B, C8G, C8orf37, C8orf46, C8orf58, C8orf59, C8orf82, C9, C9orf142, C9orf16, C9orf172, C9orf3, C9orf41, C9orf64, C9orf72, C9orf85, C9orf89, C9orf91, CA5A, CAAP1, CABIN1, CABLES1, CABLES2, CABYR, CACNA1D, CACNA1H, CACYBP, CAD, CADM1, CADM4, CADPS2, CAHM, CALCA, CALCOCO1, CALD1, CALM2, CAMK1D, CAMK2B, CAMK2D, CAMSAP2, CAND1, CANT1, CAP2, CAPG, CAPN1, CAPN15, CAPN3, CAPN5, CAPN7, CAPN8, CAPNS1, CAPRIN1, CAPRIN2, CAPZB, CARD10, CARD16, CARD6, CARD8, CARHSP1, CASC10, CASC19, CASD1, CASK, CASKIN2, CASP1, CASP3, CASP6, CASP9, CASZ1, CATSPER3, CAV1, CAV2, CBFA2T2, CBLC, CBR1, CBR3, CBR3-AS1, CBR4, CBX4, CBX5, CBX6, CBX7, CC2D1A, CC2D1B, CCAR2, CCAT1, CCDC101, CCDC102A, CCDC106, CCDC107, CCDC112, CCDC113, CCDC117, CCDC12, CCDC120, CCDC124, CCDC126, CCDC127, CCDC137, CCDC138, CCDC14, CCDC146, CCDC149, CCDC167, CCDC17, CCDC28A, CCDC28B, CCDC34, CCDC43, CCDC50, CCDC58, CCDC59, CCDC61, CCDC64, CCDC66, CCDC69, CCDC71L, CCDC85B, CCDC85C, CCDC86, CCDC88A, CCDC88B, CCDC91, CCDC92, CCDC94, CCHCR1, CCL15, CCL15-CCL14, CCL16, CCL20, CCM2, CCNB1, CCNB1IP1, CCND1, CCNE1, CCNF, CCNG1, CCNG2, CCNJ, CONY, CCP110, CCPG1, CCS, CCSAP, CCT2, CCT3, CD14, CD177, CD1D, CD24, CD320, CD44, CD46, CD55, CD58, CD68, CD81, CDA, CDAN1, CDC14B, CDC23, CDC25B, CDC26, CDC37, CDC37L1, CDC37L1-AS1, CDC40, CDC42BPA, CDC42EP1, CDC42EP2, CDC42EP3, CDC42EP4, CDC42SE2, CDC5L, CDC7, CDC73, CDCA7L, CDH1, CDH2, CDH24, CDH6, CDHR2, CDHR5, CDIP1, CDK12, CDK13, CDK14, CDK17, CDK18, CDK19, CDK20, CDK2AP1, CDK2AP2, CDK4, CDK5, CDK5RAP1, CDK5RAP2, CDK6, CDK8, CDKAL1, CDKL1, CDKL3, CDKN1A, CDKN2A, CDKN2AIPNL, CDKN2B, CDKN2C, CDKN2D, CDKN3, CDO1, CDON, CDPF1, CDRT4, CDYL2, CEACAM1, CEACAM19, CEBPA, CEBPA-AS1, CEBPB, CEBPG, CEBPZ, CECR2, CELF6, CELSR1, CELSR2, CENPB, CENPBD1, CENPC, CENPH, CENPO, CENPT, CENPV, CENPW, CEP112, CEP120, CEP131, CEP152, CEP162, CEP164, CEP170B, CEP192, CEP250, CEP290, CEP295, CEP350, CEP57, CEP57L1, CEP72, CEP78, CEP83, CEP85, CEP85L, CEP97, CERS4, CERS6, CES1P1, CES3, CETN3, CFAP44, CFAP97, CFB, CFHR1, CFHR2, CFHR3, CFHR4, CFHR5, CFI, CGGBP1, CGN, CGNL1, CH17-340M24.3, CHAC1, CHAF1A, CHAMP1, CHCHD1, CHCHD10, CHCHD2, CHCHD3, CHCHD5, CHCHD6, CHCHD7, CHD1, CHD2, CHD4, CHD6, CHDH, CHEK2, CHI3L1, CHI3L2, CHIC1, CHIC2, CHMP1B, CHMP3, CHMP4C, CHMP6, CHMP7, CHN1, CHN2, CHORDC1, CHP1, CHRD, CHRDL2, CHRNB1, CHST12, CHST14, CIB2, CIC, CIDEB, CIRBP, CIRH1A, CISD3, CISH, CITED4, CIZ1, CKAP2, CKAP4, CKAP5, CKB, CKLF, CKS1B, CKS2, CLASP1, CLASP2, CLCN2, CLCN4, CLCN5, CLCN6, CLDN1, CLDN12, CLDN16, CLDN23, CLDN3, CLDN7, CLDN9, CLEC16A, CLEC2B, CLGN, CLIC1, CLIP1, CLIP2, CLIP3, CLK1, CLK2, CLOCK, CLPB, CLPTM1, CLPX, CLSTN1, CLSTN3, CLTC, CLTCL1, CLUAP1, CLUHP3, CMAS, CMBL, CMC1, CMC2, CMIP, CMPK2, CMSS1, CMTM3, CMTM7, CMTM8, CMTR1, CMTR2, CNDP1, CNDP2, CNKSR1, CNKSR3, CNN1, CNN2, CNN3, CNNM1, CNNM2, CNNM3, CNOT1, CNOT10, CNOT11, CNOT2, CNOT6L, CNOT8, CNP, CNTLN, CNTN3, CNTNAP2, CNTRL, CNTROB, COA1, COA4, COA6, COAT, COBL, COBLL1, COG1, COG2, COG5, COG6, COL16A1, COL18A1, COL27A1, COL5A2, COL5A3, COL7A1, COLQ, COMMD1, COMMD10, COMMD2, COMMD3, COMMD3-BMI1, COMMD4, COMMD6, COMTD1, COPB1, COPG2, COPS4, COPZ2, COQ10A, COQ10B, COQ5, COQ7, CORO1C, CORO2A, COR07, COTL1, COX14, COX17, COX4I1, COX5A, COX5B, COX6A1, COX6A2, COX6B1, COX6C, COX7A2, COX7B, COX7C, COX8A, CP, CPB2, CPE, CPEB3, CPEB4, CPED1, CPM, CPN2, CPNE2, CPNE8, CPPED1, CPSF2, CPSF3, CPSF4, CPTP, CRADD, CRBN, CRCT1, CREB1, CREB3L2, CREB5, CREBBP, CREBRF, CREBZF, CREG1, CRIM1, CRIP1, CRIP2, CRIPAK, CRIPT, CRNDE, CRNKL1, CROCC, CROT, CRP, CRTC1, CRTC3, CRY2, CRYAA, CRYBB2P1, CRYBB3, CRYBG3, CRYGS, CS, CSE1L, CSF1, CSNK1G1, CSPG5, CSPP1, CSRNP2, CSRP1, CSRP2, CSRP2BP, CST3, CST6, CSTA, CSTB, CSTF1, CSTF2, CSTF2T, CTBP1, CTBP1-AS2, CTBP2, CTC-338M12.4, CTC1, CTCF, CTDSP2, CTGF, CTH, CTIF, CTNNA1, CTNNBL1, CTNS, CTPS1, CTPS2, CTSB, CTSF, CTSH, CTSK, CTSL, CTSS, CTTN, CTTNBP2NL, CTU1, CTXN1, CUEDC1, CUEDC2, CUL1, CUL2, CUL4B, CUL5, CUL7, CUL9, CUTC, CUX1, CWC27, CWF19L2, CXCL6, CXXC5, CXorf40A, CXorf57, CYB561D1, CYB561D2, CYB5A, CYB5D1, CYB5R3, CYB5RL, CYGB, CYP11A1, CYP21A1P, CYP27A1, CYP2A6, CYP2B6, CYP2C18, CYP2C8, CYP2D6, CYP2D7, CYP2E1, CYP2R1, CYP39A1, CYP4A11, CYP4F11, CYP4F12, CYP4F2, CYP4F22, CYP4F3, CYP51A1, CYP7B1, CYR61, CYTH3, DAAM1, DAB2, DAB2IP, DAG1, DANCR, DAO, DAPK1, DAPK2, DARS2, DAZAP1, DBF4B, DBH-AS1, DBI, DBN1, DBNDD1, DBP, DBR1, DBT, DCAF16, DCAF6, DCBLD1, DCBLD2, DCDC2, DCLRE1A, DCLRE1B, DCP2, DCPS, DCTN1, DCTPP1, DCUN1D1, DCUN1D2, DCUN1D4, DCXR, DDAH1, DDAH2, DDB2, DDC, DDHD1, DDI2, DDIT4, DDO, DDR1, DDT, DDX10, DDX11, DDX17, DDX20, DDX23, DDX39B, DDX3X, DDX42, DDX5, DDX52, DDX56, DDX58, DDX59, DDX6, DDX60, DEDD, DEFB1, DEFB135, DENND1A, DENND1B, DENND2C, DENND2D, DENND4A, DENND4B, DENND4C, DENND5B, DENND6A, DENR, DEPDC5, DEPDC7, DEPTOR, DESI2, DFFB, DFNA5, DFNB31, DGAT2, DGCR14, DGCR6L, DGKA, DGKD, DGKE, DGKH, DHCR24, DHCR7, DHODH, DHRS13, DHRS3, DHRS4-AS1, DHRS4L2, DHRS9, DHX15, DHX29, DHX30, DHX33, DHX36, DHX37, DHX40, DHX57, DHX8, DHX9, DIAPH1, DIAPH2, DIAPH3, DIEXF, DIMT1, DIO1, DIP2B, DIP2C, DIRC2, DIS3L, DIS3L2, DISP1, DIXDC1, DKC1, DKFZP586I1420, DKFZp779M0652, DKK3, DLAT, DLC1, DLD, DLG1, DLG5, DLGAP1-AS2, DLGAP4, DMD, DMGDH, DMKN, DMPK, DMRTA1, DMTN, DMXL1, DMXL2, DNAAF1, DNAAF2, DNAAF5, DNAH1, DNAH14, DNAJA1, DNAJA2, DNAJB12, DNAJB4, DNAJB5, DNAJB9, DNAJC11, DNAJC14, DNAJC17, DNAJC28, DNAJC3, DNAJC30, DNALI1, DNHD1, DNLZ, DNM1L, DNM2, DNMBP, DNMT1, DNMT3A, DNPH1, DNTTIP2, DOCK1, DOCK4, DOCKS, DOCK7, DOCK9, DOHH, DOK3, DOK7, DOLK, DOLPP1, DONSON, DOPEY1, DOPEY2, DPCD, DPF3, DPH1, DPH2, DPH5, DPH6, DPM2, DPM3, DPP3, DPP9, DPY19L1, DPY30, DPYD, DPYS, DPYSL2, DRAP1, DRG1, DROSHA, DSC2, DSCC1, DSE, DSEL, DSN1, DST, DSTN, DSTYK, DTD1, DTNA, DTNB, DTNBP1, DTWD1, DTWD2, DTX1, DTX3L, DTX4, DTYMK, DUS2, DUS3L, DUSP1, DUSP10, DUSP11, DUSP12, DUSP16, DUSP18, DUSP23, DUSP5, DUSP6, DUSP7, DUSP8, DVL1, DYM, DYNC1H1, DYNC1LI1, DYNC2LI1, DYNLL1, DYNLT1, DYRK1B, DYRK3, DYRK4, DYSF, DZIP3, E2F5, EARS2, EBAG9, EBP, EBPL, ECD, ECE1, ECE2, ECHDC2, ECI1, ECT2, EDA2R, EDARADD, EDC4, EDEM1, EDEM3, EDF1, EDN2, EDRF1, EEA1, EED, EEF1A2, EEF1D, EEF2K, EEFSEC, EEPD1, EFCAB11, EFCAB7, EFHD1, EFNA2, EFNA4, EFNB1, EFTUD2, EGFR, EGLN1, EGLN3, EGOT, EGR1, EHBP1, EHBP1L1, EHD1, EHD2, EHD4, EHHADH, EHMT1, EHMT2, EID2B, EIF2AK2, EIF2AK4, EIF2B3, EIF2B4, EIF2B5, EIF2S2, EIF3IP1, EIF3J-AS1, EIF4A1, EIF4A2, EIF4B, EIF4EBP1, EIF4G1, EIF4G3, EIF4H, EIF5B, ELAC2, ELF1, ELF2, ELF3, ELFN1, ELK1, ELK3, ELL, ELMO1, ELMSAN1, ELOF1, ELOVL1, ELOVL6, ELP3, ELP6, EMC1, EMC2, EME2, EMG1, EML2-AS1, EML3, EML4, EMP3, ENAH, ENAM, ENC1, ENDOD1, ENDOG, ENGASE, ENHO, ENO1, ENOX2, ENPEP, ENPP1, ENPP2, ENPP4, ENTHD2, ENTPD4, ENTPD5, ENTPD8, EP300, EP300-AS1, EP400NL, EPAS1, EPB41, EPB41L2, EPB41L4A, EPB41L4A-AS1, EPB41L5, EPC1, EPC2, EPG5, EPHA2, EPHB4, EPHB6, EPHX1, EPM2AIP1, EPOR, EPPK1, EPS15L1, EPS8, ERAL1, ERBB2, ERBB2IP, ERBB3, ERC1, ERCC2, ERCC3, ERCC4, ERCC5, ERCC6, ERCC6-PGBD3, ERCC6L2, ERF, ERGIC1, ERI2, ERI3, ERICH1, ERICH2, ERMP1, ERN1, ERO1B, ERP29, ESCO1, ESPN, ESRP2, ESRRA, ETAA1, ETFB, ETHE1, ETS1, ETV1, ETV3, ETV4, ETV5, ETV6, ETV7, EVA1A, EVA1B, EVC, EVI5, EVI5L, EVL, EWSR1, EXD2, EXO5, EXOC2, EXOC3-AS1, EXOC3L4, EXOC4, EXOC6, EXOC6B, EXOC7, EXOG, EXOSC10, EXOSC2, EXOSC3, EXOSC4, EXOSC5, EXOSC6, EXOSC8, EXOSC9, EXPH5, EXT1, EXTL2, EXTL3, EZH2, F12, F13B, F2, F2R, F2RL1, F3, F5, F7, F9, FAAH2, FAAP20, FAAP24, FADD, FADS3, FAF1, FAHD2CP, FAM102A, FAM102B, FAM104A, FAM109A, FAM110A, FAM110B, FAM110C, FAM111A, FAM114A1, FAM114A2, FAM117A, FAM117B, FAM118A, FAM122A, FAM126A, FAM129B, FAM133B, FAM135A, FAM13B, FAM13C, FAM160A2, FAM160B2, FAM168A, FAM168B, FAM169A, FAM171A1, FAM172A, FAM173A, FAM174A, FAM175A, FAM178A, FAM179B, FAM184A, FAM185A, FAM188A, FAM188B, FAM189B, FAM193A, FAM195A, FAM195B, FAM199X, FAM201A, FAM207A, FAM208A, FAM208B, FAM20B, FAM20C, FAM212A, FAM212B, FAM213B, FAM214A, FAM214B, FAM219B, FAM21A, FAM21C, FAM221A, FAM222A, FAM222B, FAM24B, FAM45A, FAM45B, FAM46A, FAM50A, FAM53C, FAM57A, FAM63A, FAM65A, FAM69B, FAM73B, FAM78B, FAM83G, FAM83H, FAM84B, FAM86B3P, FAM86C1, FAM86C2P, FAM86EP, FAM86JP, FAM89B, FAM8A1, FAM92A1, FAM95C, FAM96B, FAM98A, FAM98B, FAM99A, FAN1, FANCC, FANCF, FANCL, FARP1, FARS2, FARSA, FARSB, FASTKD1, FASTKD2, FASTKD5, FAT1, FAT4, FAU, FAXDC2, FBF1, FBL, FBLIM1, FBRS, FBRSL1, FBXL14, FBXL15, FBXL17, FBXL18, FBXL19, FBXL19-AS1, FBXL20, FBXL3, FBXL5, FBXL6, FBXL7, FBXO16, FBXO17, FBXO18, FBXO2, FBXO21, FBXO25, FBXO32, FBXO33, FBXO34, FBXO36, FBXO38, FBXO4, FBXO44, FBXO6, FBXW11, FBXW8, FCER1G, FCGRT, FCHSD1, FCHSD2, FDFT1, FDXACB1, FDXR, FER, FGA, FGB, FGD1, FGD4, FGD6, FGF13, FGF14, FGFR2, FGFR4, FGFRL1, FGG, FGL1, FHL3, FHOD1, FICD, FIG4, FIGN, FIGNL1, FILIP1L, FIP1L1, FIS1, FITM2, FIZ1, FJX1, FKBP14, FKBP15, FKBP2, FKBP4, FKBPL, FKRP, FKTN, FLAD1, FLG-AS1, FLI1, FLJ20021, FLJ42351, FLJ46906, FLNB, FLYWCH1, FLYWCH2, FMNL2, FMO4, FMO5, FMR1, FN1, FN3K, FNBP1, FNBP1L, FNDC3A, FNDC3B, FNDC5, FNIP1, FNIP2, FOCAD, FOLH1, FOS, FOSB, FOXA1, FOXA3, FOXD2-AS1, FOXN2, FOXN3, FOXO1, FOXO4, FOXP2, FOXP4, FOXRED2, FPGT, FRA10AC1, FRAT1, FRAT2, FRG1, FRK, FRMD4A, FRMD4B, FRMD6, FRMD8, FRRS1, FRY, FRYL, FSCN1, FST, FSTL1, FTCD, FTCDNL1, FTH1, FTO, FTSJ1, FTSJ3, FTX, FUBP1, FUCA1, FUK, FUNDC1, FUOM, FURIN, FUT11, FXN, FXR1, FXYD1, FXYD5, FYCO1, FZD8, G2E3, G3BP1, G6PC3, G6PD, GAB1, GAB2, GABARAPL1, GABPB1-AS1, GABRE, GADD45A, GADD45G, GADD45GIP1, GAL, GALC, GALK1, GALK2, GALM, GALNT10, GALNT15, GALNT18, GALNT2, GALNT4, GALT, GANAB, GANC, GAREM, GARNL3, GARS, GART, GAS2, GAS2L1, GAS5, GATA4, GATA6, GATA6-AS1, GATAD2A, GATB, GATM, GBA, GBA3, GBE1, GBF1, GBP2, GBP7, GC, GCFC2, GCGR, GCH1, GCHFR, GCKR, GCLC, GCLM, GCN1, GCNT2, GCNT3, GCNT4, GCSH, GDA, GDAP2, GDF7, GDNF, GDPD1, GDPD5, GEMIN2, GEMIN4, GEMIN5, GEMIN6, GEMIN7, GEN1, GFOD1, GFOD2, GFPT1, GFRA1, GGA2, GGA3, GGCX, GGPS1, GGT1, GGT7, GIMAP2, GINS2, GINS3, GIPC1, GIPC2, GIT1, GIT2, GJB1, GJB2, GJB3, GKAP1, GLB1L, GLCCI1, GLCE, GLDC, GLG1, GLIPR1, GLIPR2, GLIS3, GLMN, GLRB, GLRX, GLS, GLS2, GLT1D1, GLTP, GLTPD2, GLTSCR1, GLTSCR1L, GLUD2, GLYAT, GLYATL1, GLYCTK, GMCL1, GMDS, GMIP, GMNN, GMPPB, GMPR, GMPS, GNA12, GNA14, GNAI1, GNAO1, GNAQ, GNB1, GNB1L, GNE, GNG12, GNG7, GNL3, GNL3L, GNPDA1, GNPDA2, GNPNAT1, GNPTAB, GOLGB1, GOLIM4, GOLPH3, GOLPH3L, GON4L, GOPC, GORAB, GORASP2, GOSR1, GOT2, GPAM, GPATCH1, GPATCH2, GPC1, GPC6, GPCPD1, GPD1, GPD1L, GPD2, GPER1, GPHN, GPKOW, GPNMB, GPR135, GPR137B, GPR153, GPR155, GPR157, GPR176, GPR180, GPR39, GPRC5A, GPRC5B, GPRC5C, GPRIN1, GPT, GPT2, GPX1, GPX2, GPX7, GRAMD1A, GRAMD1C, GRAMD3, GRB10, GRB14, GRB7, GREB1L, GRHL1, GRID1, GRIPAP1, GRK5, GRTP1, GRWD1, GSAP, GSDMB, GSK3A, GSK3B, GSPT2, GSTA1, GSTA2, GSTM2, GSTM3, GSTM4, GST02, GSTP1, GTDC1, GTF2A1, GTF2B, GTF2E2, GTF2F2, GTF2H2C_2, GTF2I, GTF2IRD1, GTF3C1, GTF3C2, GTF3C3, GTF3C5, GTPBP2, GTPBP3, GTPBP8, GUCA1B, GUCA2B, GUCY1A3, GUCY1B3, GUK1, GUSB, GUSBP11, GXYLT1, GXYLT2, GYG1, GYG2, GYPC, GYS1, H19, H1F0, H1FX, H2AFJ, H2AFV, H2AFX, H2AFY2, H2AFZ, H3F3B, H3F3C, H6PD, HABP2, HACD1, HACD2, HACE1, HACL1, HAGHL, HAL, HAO1, HARBI1, HARS, HARS2, HAS3, HAUS1, HAUS3, HAUS4, HAUS7, HAUS8, HAVCR1, HAX1, HBB, HBP1, HCFC1, HCFC1R1, HCFC2, HCG18, HCG8, HCN3, HCP5, HDAC1, HDAC2, HDAC4, HDAC5, HDAC7, HDAC8, HDDC2, HDGFRP2, HDHD1, HDHD3, HDX, HEATR1, HEATR3, HEATR5A, HEATR5B, HEATR6, HECA, HECTD1, HECTD2, HECTD4, HEG1, HELLS, HELZ, HELZ2, HEMK1, HERC1, HERC2, HERC2P2, HERC3, HERC5, HERC6, HERPUD1, HERPUD2, HES1, HES2, HES4, HES6, HEXIM1, HEXIM2, HHEX, HIC2, HID1, HIF1A, HIF1AN, HIGD1A, HIGD2A, HILPDA, HINT1, HINT2, HIP1, HIP1R, HIPK2, HIRIP3, HIST1H1C, HIST1H1E, HIST1H2AC, HIST1H2AE, HIST1H2BC, HIST1H2BD, HIST1H2BJ, HIST1H2BK, HIST1H2BN, HIST1H3D, HIST1H3E, HIST1H3H, HIST1H4H, HIST2H2BE, HIST4H4, HIVEP1, HIVEP3, HLA-B, HLA-DMA, HLA-DRA, HLA-E, HLF, HLTF, HLX, HMBS, HMCES, HMG20A, HMGB2, HMGN3, HMGN4, HMGN5, HMGXB3, HMOX2, HN1, HNF1A, HNF1A-AS1, HNF1B, HNF4A, HNF4G, HNMT, HNRNPA1, HNRNPA1L2, HNRNPA1P33, HNRNPA2B1, HNRNPAB, HNRNPD, HNRNPDL, HNRNPH3, HNRNPL, HNRNPLL, HNRNPM, HNRNPR, HNRNPU, HNRNPU-AS1, HOMER3, HOMEZ, HOOK2, HOOK3, HP, HPCAL1, HPD, HPN, HPR, HPS4, HPS5, HPS6, HPX, HRAS, HRG, HRSP12, HS2ST1, HS3ST3B1, HS6ST1, HSD11B1, HSD11B2, HSD17B10, HSD17B13, HSD17B14, HSD17B2, HSD17B3, HSD17B6, HSD17B7P2, HSD3B7, HSF2, HSP90AA1, HSP90B1, HSP90B2P, HSPA13, HSPA4, HSPA4L, HSPA8, HSPB9, HSPBAP1, HSPD1, HSPG2, HTRA1, HTT, HULC, HUWE1, HYAL1, HYAL2, HYLS1, HYOU1, ICAM1, ICE2, ICK, ICMT, ICOSLG, ID1, ID2, ID3, IDH1, IDH1-AS1, IDH2, IDH3A, ID12-AS1, IDNK, IDUA, IER2, IER5, IER5L, IFF02, IFI16, IFI27L1, IFI27L2, IFI30, IFI44, IFI6, IFIH1, IFIT3, IFITM2, IFNAR2, IFNGR2, IFRD1, IFRD2, IFT122, IFT140, IFT172, IFT20, IFT46, IFT74, IFT80, IFT81, IFT88, IGF1, IGF2, IGF2R, IGFALS, IGFBP2, IGFBP4, IGFLR1, IGIP, IGSF3, IGSF9, IKBIP, IKBKAP, IKBKE, IKBKG, IL11, IL11RA, IL15, IL15RA, IL17RA, IL17RE, IL1R1, IL1R2, IL1RAP, IL1RL1, IL1RN, IL2ORB, IL27RA, IL32, IL4R, IL6R, IL7, ILDR2, ILF3, IMMP2L, IMP3, IMPDH1, IMPDH2, INADL, INAFM1, INAFM2, INCENP, INF2, ING1, ING2, ING3, ING4, INHBB, INHBC, INIP, INO80, INO80B, INO80C, INPP1, INPP4A, INPP5A, INPP5B, INPPL1, INS-IGF2, INSIG2, INSR, INTS3, INTS4, INTS5, INTS6, INTS6-AS1, INTS7, INTS8, INVS, IP6K3, IPO11, IPO13, IPO4, IPO5P1, IPO8, IPP, IPW, IQCB1, IQCE, IQCJ-SCHIP1, IQCK, IQGAP2, IQSEC1, IRAK2, IRAK4, IREB2, IRF1, IRF2, IRF2BP1, IRF2BPL, IRF3, IRF6, IRF7, IRF8, IRF9, IRS1, IRS2, ISCA1, ISCA2, ISCU, ISG15, ISG20, ISM1, ISOC2, ISYNA1, ITGA1, ITGA2, ITGA6, ITGAM, ITGAV, ITGB1, ITGB3, ITGB5, ITGB7, ITIH1, ITIH2, ITIH3, ITIH4, ITM2C, ITPA, ITPK1, ITPKA, ITPR1, ITPR2, ITPR3, ITPRIPL2, ITSN1, IVNS1ABP, IWS1, IYD, IZUMO4, JADE1, JADE2, JADE3, JAG1, JAK1, JAK2, JARID2, JAZF1, JDP2, JHDM1D-AS1, JMJD1C, JMJD4, JMY, JOSD1, JOSD2, JPX, JUNB, JUND, JUP, KALRN, KANK2, KANK4, KANSL1, KANSL1L, KANSL2, KANSL3, KAT2A, KAT2B, KAT5, KAT6A, KAT6B, KAT7, KATNBL1, KBTBD11, KBTBD6, KBTBD7, KBTBD8, KCCAT211, KCNAB3, KCNC3, KCND3, KCNK1, KCNMA1, KCNN2, KCNS3, KCNT2, KCTD1, KCTD10, KCTD11, KCTD13, KCTD15, KCTD17, KCTD20, KCTD5, KCTD7, KDELC2, KDF1, KDM1A, KDM2B, KDM3A, KDM4A, KDM4B, KDM4C, KDM5B, KDM5C, KDM6A, KDM7A, KDM8, KEAP1, KHDRBS3, KHK, KHNYN, KIAAOC20, KIAA0040, KIAA0100, KIAA0196, KIAA0232, KIAA0391, KIAA0513, KIAA0556, KIAA0586, KIAA0895, KIAA0895L, KIAA0922, KIAA1033, KIAA1107, KIAA1109, KIAA1147, KIAA1161, KIAA1279, KIAA1407, KIAA1467, KIAA1468, KIAA1549, KIAA1551, KIAA1586, KIAA1671, KIAA1804, KIAA1841, KIAA1919, KIAA1958, KIF12, KIF13A, KIF13B, KIF16B, KIF1B, KIF1C, KIF20B, KIF21A, KIF21B, KIF22, KIF3A, KIF3B, KIF5B, KIFC2, KIFC3, KISS1, KIZ, KLF10, KLF12, KLF16, KLF4, KLF5, KLF6, KLF7, KLHDC10, KLHDC2, KLHDC4, KLHDC7A, KLHDC8B, KLHDC9, KLHL12, KLHL13, KLHL15, KLHL21, KLHL22, KLHL23, KLHL24, KLHL25, KLHL26, KLHL28, KLHL29, KLHL3, KLHL36, KLHL42, KLHL7, KLHL8, KLHL9, KLKB1, KMO, KMT2C, KMT2D, KMT2E-AS1, KNDC1, KNG1, KNOP1, KNSTRN, KNTC1, KPNA2, KPNA3, KPNA7, KPNB1, KPTN, KRBA1, KRBA2, KREMEN1, KRIT1, KRT18, KRT19, KRT222, KRT8, KRTAP5-AS1, KSR1, KYNU, L3HYPDH, L3MBTL2, L3MBTL3, L3MBTL4, LACC1, LACE1, LACTB, LAD1, LAMA5, LAMB2, LAMC1, LAMC2, LAMTOR4, LANCL1, LANCL2, LAPTM5, LARGE, LARP6, LARP7, LARS2, LAS1L, LASP1, LATS1, LBHD1, LBP, LBX2, LBX2-AS1, LCAT, LCMT1, LCMT2, LCN12, LCN2, LCORL, LCP1, LDB1, LDHB, LDHD, LDLR, LDLRAD3, LDLRAP1, LDOC1L, LEAP2, LEMD3, LENG8, LENG8-AS1, LEO1, LETM1, LGALSL, LGR4, LHFPL2, LHX4-AS1, LIG3, LIMA1, LIMCH1, LIME1, LIMK1, LIMS1, LIMS2, LIN54, LIN7A, LIN7B, LIN7C, LINC-PINT, LINC00094, LINC00116, LINC00312, LINC00313, LINC00339, LINC00342, LINC00467, LINC00482, LINC00493, LINC00525, LINC00526, LINC00630, LINC00641, LINC00649, LINC00657, LINC00663, LINC00842, LINC00844, LINC00857, LINC00862, LINC00884, LINC00888, LINC00899, LINC00926, LINC00938, LINC00969, LINC01000, LINC01003, LINC01004, LINC01018, LINC01021, LINC01089, LINC01093, LINC01125, LINC01137, LINC01146, LINC01176, LINC01268, LINC01270, LINC01273, LINC01311, LINC01314, LINC01315, LINC01320, LINC01355, LINC01370, LINC01372, LINC01474, LINC01485, LINC01534, LINC01547, LINC01564, LINC01573, LINC01578, LINC01588, LINC01604, LIPC, LIPG, LIPH, LIPT1, LLGL2, LMAN1, LMBR1, LMBRD2, LMF2, LMLN, LMNB1, LMNB2, LMNTD2, LMO4, LMO7, LMTK2, LNPEP, LNX1, LOC100130744, LOC100131564, LOC100132111, LOC100132352, LOC100268168, LOC100270746, LOC100288152, LOC100288798, LOC100294145, LOC100419583, LOC100422737, LOC100505942, LOC100506022, LOC100506302, LOC100506476, LOC100506548, LOC100506603, LOC100506675, LOC100506746, LOC100506844, LOC100506860, LOC100506990, LOC100507389, LOC100507487, LOC100652758, LOC100996437, LOC101926944, LOC101927021, LOC101927045, LOC101927136, LOC101927311, LOC101927391, LOC101927481, LOC101927571, LOC101927746, LOC101927755, LOC101927934, LOC101928303, LOC101928370, LOC101928767, LOC101929147, LOC101929715, LOC102606465, LOC102723766, LOC102724814, LOC103344931, LOC105375734, LOC113230, LOC115110, LOC146880, LOC171391, LOC284454, LOC339803, LOC388692, LOC389332, LOC389602, LOC389765, LOC401010, LOC407835, LOC440311, LOC642236, LOC644656, LOC644936, LOC646762, LOC653160, LOC728554, LOC728730, LOC730101, LOC730102, LOC730202, LOC90784, LOH12CR1, LONRF1, LONRF2, LPA, LPCAT1, LPCAT3, LPCAT4, LPIN1, LPIN3, LPP, LPPR2, LPXN, LRBA, LRCH1, LRCH3, LRCH4, LRFN3, LRG1, LRIG1, LRIG2, LRIG3, LRP1, LRP10, LRP11, LRP3, LRP5, LRP6, LRPAP1, LRR1, LRRC16A, LRRC2, LRRC20, LRRC27, LRRC28, LRRC31, LRRC40, LRRC45, LRRC57, LRRC61, LRRC75A-AS1, LRRC75B, LRRC8A, LRRC8B, LRRC8D, LRRC8E, LRSAM1, LSM10, LSM2, LSM3, LSM6, LSMEM1, LSR, LSS, LTB, LTBP4, LTC4S, LTN1, LTV1, LUC7L, LUC7L2, LURAP1L, LVCAT8, LY6E, LY6G5B, LY96, LYAR, LYG1, LYN, LYNX1, LYPLA2, LYPLAL1, LYRM4, LYRM9, LYSMD2, LYSMD3, LYSMD4, LYST, LZIC, LZTR1, LZTS2, LZTS3, M6PR, MAATS1, MACF1, MACROD1, MAD1L1, MAF, MAF1, MAFG, MAFK, MAGED1, MAGEF1, MAGEH1, MAGI1, MAGI3, MAGIX, MALAT1, MALSU1, MALT1, MAMDC4, MAML3, MAMLD1, MAN1A1, MAN1B1-AS1, MAN2A1, MAN2A2, MAN2B1, MANBAL, MANEA, MANEAL, MANSC1, MAOB, MAP10, MAP1LC3A, MAP1LC3B, MAP1LC3B2, MAP2, MAP2K3, MAP2K4, MAP2K5, MAP2K7, MAP3K10, MAP3K11, MAP3K14, MAP3K2, MAP3K3, MAP3K4, MAP3K5, MAP3K6, MAP3K7CL, MAP3K9, MAP4, MAP4K2, MAP4K3, MAP4K4, MAP7, MAP7D1, MAP7D3, MAPK11, MAPK12, MAPK14, MAPK1IP1L, MAPK8IP3, MAPKAP1, MAPKAPK5-AS1, MAPRE3, MAPT, MARC1, MARCH2, MARCH6, MARCH8, MARCH9, MARCKS, MARCKSL1, MARS2, MARVELD1, MARVELD3, MASP1, MASP2, MAST2, MAST3, MASTL, MAT1A, MAT2A, MAT2B, MAVS, MB, MBD3, MBD4, MBD5, MBD6, MBL1P, MBL2, MBLAC1, MBLAC2, MBNL1, MBNL2, MBNL3, MBOAT1, MBP, MBTD1, MBTPS2, MCC, MCCC1, MCCC2, MCF2L, MCF2L-AS1, MCHR1, MCM3, MCM3AP, MOMS, MCM6, MCM9, MCMBP, MCPH1, MCRS1, MCU, MDFIC, MDK, MDM1, MDM2, MDN1, ME2, ME3, MED1, MED11, MED12, MED13L, MED14, MED17, MED19, MED21, MED23, MED24, MED25, MED27, MED30, MED6, MED8, MEF2A, MEGF6, MEGF9, MEIS1, MEIS2, MELK, MEMO1, MEN1, MEPCE, MERTK, MESDC1, MESP1, MET, METAP1D, METTL1, METTL13, METTL14, METTL15, METTL18, METTL20, METTL23, METTL25, METTL4, METTL5, METTL6, METTL7A, METTL7B, METTL8, METTL9, MEX3A, MEX3D, MFAP1, MFAP3L, MFGE8, MFHAS1, MFI2, MFSD10, MFSD2A, MFSD3, MFSDS, MFSD6, MFSD7, MGA, MGAM, MGAT2, MGAT4A, MGATS, MGC27345, MGC57346, MGLL, MGME1, MGMT, MGST1, MIA2, MIA3, MIB1, MIB2, MICA, MICAL1, MICAL2, MICAL3, MICALL2, MICB, MICU1, MICU3, MID1, MID1IP1, MID2, MIDN, MIER2, MIER3, MIF, MIIP, MINA, MINK1, MINOS1, MIOS, MIS12, MIS18BP1, MITF, MKL1, MKL2, MKLN1, MKNK2, MKRN2, MKRN9P, MKS1, MLEC, MLF2, MLH1, MLH3, MLIP, MLKL, MLLT10, MLLT3, MLLT6, MLPH, MLX, MLXIPL, MMM, MMAB, MMADHC, MMD, MME, MMP10, MMP14, MMP15, MMP7, MN1, MNAT1, MOB1B, MOB3A, MOCS1, MOGS, MOK, MON1A, MON2, MORC4, MORF4L2, MORN4, MOV10, MPDZ, MPG, MPHOSPH10, MPHOSPH6, MPP6, MPP7, MPRIP, MPST, MPZ, MPZL2, MR1, MRAS, MRFAP1L1, MRI1, MRM1, MROH1, MROH6, MRPL11, MRPL12, MRPL14, MRPL15, MRPL16, MRPL17, MRPL2, MRPL20, MRPL22, MRPL23, MRPL27, MRPL3, MRPL33, MRPL36, MRPL41, MRPL51, MRPL52, MRPL53, MRPL9, MRPS12, MRPS18B, MRPS18C, MRPS2, MRPS23, MRPS24, MRPS26, MRPS28, MRPS30, MRPS31, MRPS31P5, MRPS34, MRPS6, MRRF, MRTO4, MSANTD3, MSH2, MSH3, MSH5, MSH6, MSI2, MSL3P1, MSN, MSRA, MSRB1, MSRB3, MST1, MST1L, MST1P2, MST1R, MSTO1, MSX1, MT1A, MT1B, MT1DP, MT1E, MT1F, MT1G, MT1H, MT1HL1, MT1JP, MT1L, MT1M, MT1X, MT2A, MTA2, MTA3, MTCL1, MTCP1, MTERF1, MTERF2, MTERF3, MTFR2, MTHFD1, MTHFD1L, MTHFR, MTHFSD, MTIF2, MTM1, MTMR1, MTMR10, MTMR12, MTMR2, MTMR4, MTMR6, MTO1, MTOR, MTPAP, MTR, MTRF1, MTRNR2L8, MTRR, MTSS1, MTSS1L, MTTP, MTURN, MTUS1, MTX2, MTX3, MUC20, MUM1, MUM1L1, MUS81, MUTYH, MVB12B, MVK, MX1, MXD1, MXD4, MYADM, MYBBP1A, MYC, MYCBP, MYCBP2, MYCL, MYD88, MYDGF, MYEOV2, MYH10, MYH14, MYH3, MYH9, MYL12A, MYL5, MYL6, MYLK, MYNN, MYO15B, MYO18A, MYO1B, MYO1D, MYO1E, MYO5A, MYO5B, MYO5C, MYO7A, MYO9A, MYPOP, MYRF, MYRIP, MZT2A, MZT2B, N4BP2, N4BP2L1, N6AMT1, NAA10, NAA15, NAA16, NAA38, NAA40, NABP1, NACA2, NACC1, NACC2, NADSYN1, NAE1, NAGK, NAGS, NAIF1, NAMPT, NAP1L5, NAPEPLD, NAPSA, NARF, NARS2, NASP, NAT1, NAT10, NAT14, NAT2, NAT8, NATD1, NAV1, NAV2, NBAS, NBEAL1, NBEAL2, NBN, NBPF1, NBR1, NBR2, NCALD, NCAPD2, NCAPD3, NCAPH2, NCBP1, NCBP2-AS2, NCDN, NCEH1, NCK1, NCK2, NCKAP5, NCKAP5L, NCKIPSD, NCOA1, NCOA2, NCOA3, NCOA5, NCOA7, NCOR2, NCS1, NDC1, NDE1, NDRG3, NDRG4, NDST1, NDST2, NDUFA1, NDUFA11, NDUFA13, NDUFA2, NDUFA3, NDUFA4, NDUFA8, NDUFAB1, NDUFAF4, NDUFB1, NDUFB10, NDUFB11, NDUFB2, NDUFB3, NDUFB6, NDUFB7, NDUFB8, NDUFC2, NDUFC2-KCTD14, NDUFS1, NDUFS3, NDUFS4, NDUFS5, NDUFS6, NDUFS7, NDUFS8, NDUFV2, NEAT1, NEBL, NECAB3, NECAP1, NEDD1, NEDD4, NEDD4L, NEDD8, NEDD9, NEIL1, NEIL2, NEK1, NEK3, NEK6, NEK7, NEK8, NELFCD, NENF, NEO1, NET1, NEU1, NEU3, NEURL4, NEXN, NF1, NF2, NFAT5, NFATC1, NFATC2, NFATC3, NFE2L1, NRA, NAB, VIC, NFIL3, NFIX, NFKB1, NFKB2, NFKBIA, NFKBIE, NFKBIZ, NFRKB, NFU1, NFYA, NFYB, NGEF, NGFRAP1, NGLY1, NHLRC1, NHP2, NHSL1, NID1, NIF3L1, NIN, NINL, NIPAL1, NIPAL2, NIPAL3, NIPBL-AS1, NIPSNAP1, NKAP, NKG7, NKX3-1, NLE1, NLGN2, NLK, NLRC5, NLRX1, NME1, NME3, NME6, NMI, NNMT, NOB1, NOC2L, NOC3L, NOCT, NOD1, NOD2, NOL10, NOL11, NOL12, NOL4L, NOL6, NOL7, NOL8, NOL9, NOLC1, NONO, NOP10, NOP14, NOP14-AS1, NOP16, NOP2, NOP56, NOP9, NOS1AP, NOSIP, NOTCH1, NOTCH2, NOXA1, NPAT, NPC1, NPC1L1, NPDC1, NPFF, NPHP4, NPIPA1, NPM1, NPM2, NPM3, NPR2, NPRL3, NPTX2, NPW, NQO1, NROB2, NR1D1, NR1H3, NR1H4, NR1I2, NR2C1, NR2F1, NR2F2, NR2F6, NR3C1, NR3C2, NR4A1, NR5A2, NRBP1, NRDE2, NREP, NRG1, NRM, NRP1, NRP2, NRSN2, NRTN, NSD1, NSMAF, NSMCE1, NSMCE2, NSMCE4A, NSUN2, NSUN4, NSUN6, NT5C3A, NT5E, NTHL1, NTMT1, NTN4, NUBP1, NUBPL, NUCKS1, NUDC, NUDT1, NUDT12, NUDT14, NUDT16, NUDT18, NUDT19, NUDT2, NUDT22, NUDT3, NUDT4, NUDT7, NUDT8, NUFIP2, NUGGC, NUMA1, NUMB, NUMBL, NUP107, NUP133, NUP155, NUP160, NUP188, NUP205, NUP210, NUP214, NUP35, NUP37, NUP43, NUP50-AS1, NUP88, NUP93, NUP98, NUPL2, NUPR1, NUSAP1, NXF1, NXPH4, NXT1, NYNRIN, OAT, OBSL1, OCIAD2, OCRL, ODF2L, ODF3B, OGDH, OGDHL, OGFRL1, OGG1, OIP5-AS1, OLA1, OLFM2, OLMALINC, OMA1, ONECUT1, OPA1, OPA3, OR7E14P, ORM, ORC2, ORC3, ORC4, OROS, ORM1, ORM2, OSBP, OSBPL11, OSBPL1A, OSBPL3, OSBPL6, OSBPL8, OSBPL9, OSER1, OSER1-AS1, OSGEPL1, OST4, OTC, OTUB2, OTUD1, OTUD6B-AS1, OVCA2, OVGP1, OXLD1, OXNAD1, OXSR1, OXT, P2RX4, P2RX7, P2RY11, P2RY2, P3H1, PA2G4, PA2G4P4, PAAF1, PABPC1L, PACRGL, PACS1, PACSIN2, PACSIN3, PAF1, PAFAH1B1, PAFAH1B3, PAG1, PAH, PAIP2, PAIP2B, PAK1IP1, PALLD, PALM2, PALM2-AKAP2, PALM3, PAN3, PANK2, PANK3, PANX2, PAOX, PAPD5, PAPOLG, PAPSS1, PAPSS2, PAQR3, PAQR4, PAQR6, PAQR7, PAQR9, PARD3, PARD3-AS1, PARD3B, PARD6A, PARD6B, PARN, PARP10, PARP11, PARP12, PARP16, PARP2, PARP4, PARP8, PARP9, PARTICL, PATZ1, PAWR, PAX8, PAIP1, PAIP1-AS1, PAXIP1-AS2, PBDC1, PBRM1, PBX1, PBX2, PBX3, PC, PCBP1-AS1, PCBP4, PCCA, PCDH1, PCDH9, PCDHGC3, PCGF3, PCIF1, PCK2, PCM1, PCMTD1, PCNA, PCNT, PCNX, PCNXL3, PCOLCE, PCOLCE2, PCP4L1, PCSK5, PCSK6, PCSK7, PCYT1A, PCYT2, PDCD10, PDCD11, PDCD2L, PDCD4, PDCD5, PDCL, PDE11A, PDE12, PDE3B, PDE4A, PDE4D, PDE5A, PDE6D, PDE7A, PDE9A, PDGFA, PDGFC, PDGFRA, PDIA3, PDIA5, PDIA6, PDIK1L, PDK2, PDK3, PDLIM1, PDLIM2, PDLIM5, PDP1, PDP2, PDPR, PDS5A, PDS5B, PDSS2, PDXDC2P, PDXK, PDXP, PDZD8, PDZK1IP1, PEAK1, PEBP1, PEG10, PELI1, PELP1, PEMT, PEPD, PER3, PES1, PET100, PET117, PEX11A, PEX12, PEX14, PFAS, PFDN1, PFDN2, PFDN5, PFDN6, PFKFB1, PFKFB2, PFKM, PFKP, PFN1, PGAM5, PGAP1, PGAP2, PGBD1, PGBD2, PGBD4, PGBD5, PGD, PGLYRP2, PGM1, PGM2L1, PGP, PGRMC1, PGRMC2, PHACTR2, PHACTR4, PHAX, PHC2, PHC3, PHEX, PHF10, PHF14, PHF19, PHF2, PHF20L1, PHF21A, PHF23, PHF3, PHF6, PHF8, PHGDH, PHIP, PHKA2, PHKB, PHLDA2, PHLDB1, PHLDB2, PHLPP1, PHLPP2, PHPT1, PHRF1, PHTF1, PHTF2, PHYH, PHYHIPL, PI4KA, PIAS2, PIBF1, PICALM, PIDD1, PIEZO1, PIEZO2, PIGA, PIGC, PIGM, PIGN, PIGS, PIGU, PIGZ, PIK3AP1, PIK3C2A, PIK3C2B, PIK3C2G, PIK3C3, PIK3CA, PIK3CB, PIK3CD, PIK3IP1, PIK3R1, PIK3R2, PIK3R3, PIK3R4, PIKFYVE, PILRB, PIK PIM2, PIN4, PINK1, PIP4K2A, PIP5K1A, PIP5K1C, PIR, PITPNC1, PITPNM2, PITRM1, PJA1, PJA2, PKD1, PKD1L2, PKD1P1, PKD2, PKDCC, PKHD1, PKI55, PKIB, PKIG, PKLR, PKM, PKMYT1, PKN1, PKN2, PKP2, PKP4, PLA1A, PLA2G12B, PLA2G15, PLA2G2A, PLA2G4B, PLA2G4C, PLA2G6, PLM, PLAC8L1, PLBD2, PLCB1, PLCB3, PLCD1, PLCD3, PLCG1, PLCG2, PLD1, PLD2, PLD6, PLEC, PLEK2, PLEKHA2, PLEKHA3, PLEKHA4, PLEKHA5, PLEKHA7, PLEKHB2, PLEKHF1, PLEKHG1, PLEKHG2, PLEKHG5, PLEKHG6, PLEKHH1, PLEKHH3, PLEKHJ1, PLEKHM1, PLEKHM1P, PLEKHM3, PLEKHN1, PLEKHO1, PLEKHO2, PLG, PLIN3, PLIN5, PLK3, PLLP, PLOD2, PLRG1, PLS1, PLS3, PLSCR3, PLSCR4, PLXNA1, PLXNA2, PLXNA3, PLXNB1, PLXND1, PMEPA1, PML, PMM1, PMS1, PMS2, PMVK, PNMA1, PNP, PNPLA2, PNPLA3, PNPLA7, PNRC1, POC1A, POC1B, POC5, POGK, POLA1, POLA2, POLD1, POLD2, POLD3, POLD4, POLE, POLE2, POLE4, POLG, POLG2, POLH, POLI, POLK, POLL, POLM, POLR1A, POLR1B, POLR1E, POLR2A, POLR2B, POLR2F, POLR2H, POLR2I, POLR2J, POLR2L, POLR2M, POLR3B, POLR3E, POLR3F, POLR3G, POLR3H, POLR3K, POLRMT, POMGNT2, POMK, PON1, PON3, POP1, POPS, PORCN, POT1, POU5F1, PPAN, PPAP2A, PPAPDC1B, PPAPDC2, PPARA, PPARG, PPARGC1A, PPARGC1B, PPFIA1, PPFIBP1, PPFIBP2, PPID, PPIL2, PPIP5K2, PPM1B, PPM1D, PPM1H, PPM1J, PPM1K, PPME1, PPP1CC, PPP1R10, PPP1R13B, PPP1R14B, PPP1R15A, PPP1R15B, PPP1R16A, PPP1R18, PPP1R1A, PPP1R1C, PPP1R27, PPP1R32, PPP1R35, PPP1R3B, PPP1R8, PPP1R9B, PPP2R1B, PPP2R3A, PPP2R5B, PPP2R5D, PPP2R5E, PPP3CA, PPP3CB, PPP4R1, PPP4R3B, PPP5C, PPP6R3, PPRC1, PPT2, PPTC7, PPWD1, PQBP1, PQLC2, PRADC1, PRAP1, PRC1, PRDM15, PRDM4, PRDX5, PREB, PRELID1, PREP, PREX1, PRG2, PRICKLE2, PRICKLE4, PRIM1, PRIM2, PRIMPOL, PRKAA1, PRKAA2, PRKAB1, PRKAB2, PRKACA, PRKACB, PRKAR1B, PRKAR2A, PRKCA, PRKCD, PRKCDBP, PRKCE, PRKCI, PRKCQ-AS1, PRKCZ, PRKD3, PRKDC, PRKRA, PRKX, PRMT1, PRMT5, PRMT9, PRNP, PROCR, PRODH2, PROS1, PROSER1, PROSER2, PROSER3, PROX1, PROZ, PRPF38A, PRPF6, PRPF8, PRPSAP1, PRPSAP2, PRR12, PRR22, PRR34-AS1, PRR5, PRR7, PRRC1, PRRC2A, PRRC2C, PRSS12, PRSS3, PRSS8, PRUNE, PSAT1, PSD4, PSEN2, PSG4, PSIP1, PSMB10, PSMB3, PSMB6, PSMB8, PSMB9, PSMC4, PSMC6, PSMD1, PSMD14, PSMD2, PSMD3, PSMD5, PSME1, PSME2, PSME3, PSME4, PSMG1, PSMG3, PSMC4, PSPC1, PSPH, PSPN, PSRC1, PSTK, PSTPIP2, PTAR1, PTBP1, PTBP2, PTCD2, PTDSS2, PTGES3, PTGFR, PTGFRN, PTGR1, PTK2, PTK2B, PTMA, PTMS, PTP4A1, PTPMT1, PTPN1, PTPN11, PTPN14, PTPN2, PTPN21, PTPN23, PTPN3, PTPN4, PTPN6, PTPN9, PTPRD, PTPRF, PTPRG, PTPRH, PTPRJ, PTPRK, PTPRM, PTPRS, PTRF, PTRH1, PTRH2, PUM1, PUM2, PURA, PUS1, PUS10, PUS3, PUS7, PUS7L, PVT1, PWP1, PWWP2A, PWWP2B, PXDC1, PXK, PXMP2, PXMP4, PXN, PYCR1, PYCRL, PYROXD2, PZP, OKI, QPCT, ORICH1, ORICH2, QRSL1, QSER1, QSOX1, QTRTD1, R3HDM1, R3HDM2, RAB10, RAB11FIP2, RAB11FIP3, RAB11FIP4, RAB13, RAB14, RAB15, RAB1B, RAB20, RAB23, RAB24, RAB27B, RAB28, RAB30-AS1, RAB37, RAB3B, RAB3GAP1, RAB3GAP2, RAB40B, RAB43, RAB5C, RAB8A, RAB9A, RABAC1, RABEP2, RABEPK, RABGAP1, RABGAP1L, RABGGTA, RABIF, RABL3, RAC3, RACGAP1, RAD50, RAD51-AS1, RAD51B, RAD51C, RAD52, RAD54B, RAET1L, RAF1, RAI1, RAI14, RAI2, RALGAPA1, RALGAPA2, RALGAPB, RALGDS, RALGPS1, RALGPS2, RALY, RAMP1, RANBP1, RANBP10, RANBP2, RANBP6, RANBP9, RAP1GAP, RAP2B, RAPGEF1, RAPGEF2, RAPGEF5, RAPGEF6, RAPGEFL1, RAPH1, RAPSN, RARA, RARA-AS1, RARRES2, RARRES3, RASA1, RASA3, RASAL2, RASEF, RASGEF1B, RASL11A, RASSF5, RASSF7, RASSF8-AS1, RAVER1, RB1, RB1CC1, RBBP5, RBCK1, RBFA, RBFOX2, RBM10, RBM12, RBM12B, RBM14, RBM15, RBM18, RBM19, RBM24, RBM28, RBM3, RBM33, RBM34, RBM38, RBM4, RBM41, RBM43, RBM45, RBM47, RBM48, RBM5, RBM7, RBMS1, RBMS2, RBMX, RBMXL1, RBP1, RBP4, RBPJ, RBPMS, RBPMS2, RBSN, RC3H2, RCAN1, RCAN3, RCBTB1, RCBTB2, RCC1, RCE1, RCN2, RCN3, RCOR1, RCOR3, RDH10, RDH14, RDH16, RDH5, RECQL4, RECQL5, REEP4, REEP6, REL, RELB, RELL2, RELT, REP15, REPS1, REPS2, RERE, RETSAT, RFC3, RFNG, RFTN1, RFWD2, RFWD3, RFX1, RFX3, RFX5, RFXANK, RGAG4, RGL1, RGL3, RGP1, RGS12, RGS14, RGS19, RGS3, RGS9, RHBDD2, RHBDF1, RHBDF2, RHBDL1, RHNO1, RHOA, RHOBTB1, RHOBTB3, RHOD, RHOT1, RHOU, RHPN1, RHPN2, RIC1, RIC8A, RIC8B, RIF1, RILP, RILPL1, RIMKLB, RIN1, RIN3, RING1, RINL, RIOK1, RIOK2, RIOK3, RIPK1, RIPK2, RLF, RMI1, RMI2, RMND1, RMND5B, RNASE1, RNASE4, RNASEH1-AS1, RNASEH2A, RNASEH2B, RNASEL, RNF103, RNF111, RNF115, RNF121, RNF122, RNF123, RNF125, RNF14, RNF144B, RNF146, RNF152, RNF167, RNF169, RNF180, RNF181, RNF187, RNF19A, RNF2, RNF207, RNF213, RNF214, RNF216, RNF217, RNF220, RNF26, RNF34, RNF38, RNF41, RNF43, RNFT1, RNGTT, RNLS, RNPC3, ROBO1, ROCK1, ROCK2, ROGDI, ROM1, ROMO1, RORC, RP2, RP9, RPA3, RPAP2, RPAP3, RPF1, RPGR, RPH3AL, RPL10, RPL11, RPL13, RPL13A, RPL13AP20, RPL13P5, RPL21P44, RPL22L1, RPL23, RPL24, RPL26, RPL26L1, RPL27A, RPL28, RPL30, RPL31, RPL32, RPL34, RPL35, RPL35A, RPL36, RPL36AL, RPL37, RPL37A, RPL38, RPL39, RPL39L, RPL41, RPL8, RPLP0, RPLP1, RPLP2, RPP14, RPP21, RPP25, RPP25L, RPP38, RPP40, RPRD2, RPS10P7, RPS12, RPS14, RPS14P3, RPS16, RPS18, RPS19, RPS19BP1, RPS2, RPS20, RPS21, RPS24, RPS25, RPS26, RPS27A, RPS28, RPS29, RPS5, RPS6KA1, RPS6KA2, RPS6KA3, RPS6KB1, RPS6KL1, RPS7, RPS8, RPS9, RPTOR, RPUSD1, RPUSD3, RPUSD4, RRAGB, RRAGD, RRAS, RRAS2, RRBP1, RREB1, RRM2B, RRN3P1, RRNAD1, RRP15, RRP1B, RRP36, RRP7BP, RRP9, RRS1, RSAD1, RSBN1L, RSC1A1, RSL24D1, RSPH3, RSPRY1, RSRC1, RSRP1, RTCA, RTEL1, RTF1, RTKN, RTN2, RTN4IP1, RTN4R, RTN4RL1, RTN4RL2, RTP4, RUFY1, RUNX1, RUSC1, RUSC2, RUVBL1, RWDD1, RWDD2B, RXRA, S100A10, S100A11, S100A13, S100A14, S100A4, S100A9, S100P, S1PR1, S1PR2, SAA1, SAA2, SAA4, SAAL1, SAC3D1, SACS, SAMD1, SAMD10, SAMD12, SAMD4A, SAMD4B, SAMD5, SAMD9, SAMD9L, SAMHD1, SAP130, SAP30, SARDH, SARS, SARS2, SASH1, SASS6, SAT1, SAT2, SATB1, SATB2, SAYSD1, SBDSP1, SBF2, SBN02, SC5D, SCAF11, SCAF8, SCAMPI, SCAMP2, SCAMP4, SCAND1, SCAPER, SCARB1, SCARF1, SCD, SCFD2, SCG5, SCGN, SCHIP1, SCLT1, SCLY, SCMH1, SCML2, SCN8A, SCN9A, SCNM1, SCNN1D, SCYL2, SCYL3, SDC1, SDC4, SDCCAG8, SDF2L1, SDHAF4, SDHAP2, SDR42E1, SDSL, SEC11C, SEC14L1, SEC14L2, SEC16B, SEC22A, SEC23IP, SEC24A, SEC24D, SEC31A, SEC31B, SEC61B, SEC61G, SECISBP2L, SEH1L, SEL1L, SEL1L3, SELENBP1, SELK, SELO, SEMA4B, SEMA4C, SEMA4F, SEMA4G, SEMA5A, SEMA6C, SENP2, SENP7, SEPHS2, SEPN1, SEPP1, SEPSECS, SEPT4, SEPT5, SEPT6, SEPT8, SEPT9, SEPW1, SERAC1, SERF2, SERGEF, SERINC5, SERPINA1, SERPINA10, SERPINA11, SERPINA3, SERPINA4, SERPINA5, SERPINA6, SERPINA7, SERPINB6, SERPINC1, SERPIND1, SERPINE1, SERPINF1, SERPINF2, SERPING1, SERPINH1, SERPINI1, SERTAD1, SESN1, SESN2, SESTD1, SETBP1, SETD2, SETDB1, SETDB2, SETMAR, SETX, SF1, SF3A1, SF3B3, SF3B4, SF3B5, SFMBT1, SFPQ, SFR1, SFT2D2, SFXN2, SFXN3, SFXN4, SFXN5, SGK1, SGK223, SGK3, SGMS1, SGMS2, SGOL2, SGSM2, SH2B2, SH2D3A, SH2D4A, SH2D5, SH3BGR, SH3BGRL2, SH3BGRL3, SH3BP2, SH3D19, SH3D21, SH3GL1, SH3KBP1, SH3PXD2A, SH3PXD2B, SH3RF1, SH3RF2, SH3TC1, SHANK2, SHB, SHF, SHFM1, SHH, SHISA4, SHMT1, SHMT2, SHPK, SHPRH, SHQ1, SHROOM1, SHROOM2, SHROOM3, SHTN1, SIDT2, SIK1, SIK2, SIKE1, SIL1, SIMC1, SIN3A, SIPA1, SIPA1L1, SIPA1L2, SIPA1L3, SIRPA, SIRT1, SIRT4, SIVA1, SIX5, SKA2, SKAP1, SKAP2, SKIL, SKIV2L2, SKP2, SLAIN1, SLC10A1, SLC10A3, SLC10A5, SLC10A7, SLC11A2, SLC12A4, SLC12A6, SLC12A7, SLC12A8, SLC12A9, SLC13A3, SLC13A5, SLC15A1, SLC16A12, SLC16A2, SLC16A4, SLC16A7, SLC17A1, SLC17A3, SLC17A4, SLC17A9, SLC18B1, SLC19A1, SLC19A2, SLC19A3, SLC1A1, SLC1A4, SLC20A1, SLC20A2, SLC22A1, SLC22A10, SLC22A15, SLC22A23, SLC22A3, SLC22A5, SLC22A9, SLC23A2, SLC25A1, SLC25A10, SLC25A11, SLC25A13, SLC25A14, SLC25A15, SLC25A18, SLC25A19, SLC25A22, SLC25A25, SLC25A25-AS1, SLC25A27, SLC25A28, SLC25A29, SLC25A32, SLC25A33, SLC25A40, SLC25A45, SLC25A46, SLC26A1, SLC26A11, SLC26A2, SLC26A6, SLC27A1, SLC27A2, SLC27A3, SLC27A5, SLC28A1, SLC29A2, SLC29A3, SLC2A1, SLC2A10, SLC2A12, SLC2A13, SLC2A4RG, SLC2A6, SLC2A8, SLC2A9, SLC30A1, SLC30A6, SLC31A1, SLC33A1, SLC35A4, SLC35A5, SLC35B4, SLC35C1, SLC35D2, SLC35E4, SLC35F2, SLC35F6, SLC35G1, SLC35G2, SLC36A1, SLC37A1, SLC37A3, SLC38A1, SLC38A3, SLC38A4, SLC38A9, SLC39A10, SLC39A11, SLC39A14, SLC39A4, SLC39A5, SLC3A2, SLC40A1, SLC41A1, SLC41A2, SLC43A1, SLC43A2, SLC43A3, SLC44A2, SLC44A3, SLC45A3, SLC46A1, SLC46A3, SLC48A1, SLC4A11, SLC4A1AP, SLC4A2, SLC4A4, SLC4A7, SLC50A1, SLC51A, SLC52A2, SLC5A3, SLC5A6, SLC5A9, SLC6A12, SLC6A8, SLC6A9, SLC7A1, SLC7A2, SLC7A6, SLC8B1, SLC9A3R1, SLC9A3R2, SLCO1B1, SLCO1B3, SLFN12, SLFN5, SLIRP, SLK, SLPI, SLX4, SLX4IP, SMAD1, SMAD3, SMAD4, SMAD6, SMAD7, SMARCA1, SMARCA2, SMARCA4, SMARCA5, SMARCAD1, SMARCAL1, SMARCB1, SMARCC1, SMARCD3, SMARCE1, SMC1A, SMC2, SMC3, SMC4, SMC5, SMCHD1, SMCO2, SMCO3, SMCO4, SMG1, SMG6, SMG8, SMG9, SMIM11, SMIM12, SMIM3, SMIM4, SMIM8, SMKR1, SMLR1, SMO, SMOX, SMPD2, SMPDL3A, SMPDL3B, SMS, SMTN, SMURF2, SMYD3, SMYD4, SNAI3-AS1, SNAP23, SNAPC1, SNAPC2, SNAPC3, SNAPC4, SND1, SNED1, SNHG1, SNHG10, SNHG12, SNHG15, SNHG17, SNHG19, SNHG21, SNHG25, SNHG5, SNHG6, SNHG7, SNHG8, SNIP1, SNN, SNRK, SNRK-AS1, SNRNP200, SNRNP25, SNRNP27, SNRNP35, SNRNP40, SNRPA, SNRPB2, SNRPD1, SNRPD2, SNRPE, SNRPF, SNRPG, SNTB1, SNTB2, SNUPN, SNX10, SNX13, SNX15, SNX18, SNX2, SNX21, SNX24, SNX27, SNX29P2, SNX30, SNX33, SNX8, SOAT1, SOBP, SOCS2-AS1, SOCS3, SOCS4, SOCS6, SOD1, SOD2, SOGA1, SORCS2, SORT1, SOS1, SOS2, SOWAHB, SOWAHC, SOX12, SOX13, SOX4, SOX5, SOX6, SP100, SP140L, SPAG1, SPAG16, SPAST, SPATA13, SPATA21, SPATA25, SPATA41, SPATA5, SPATA7, SPATS2, SPATS2L, SPECC1, SPECC1L, SPEN, SPG11, SPG7, SPHK1, SPICE1, SPIDR, SPIN1, SPIN2B, SPIN3, SPIN4, SPINK1, SPINT1, SPIRE2, SPON2, SPOP, SPP2, SPRED1, SPRED2, SPRTN, SPRY4, SPSB1, SPSB3, SPTAN1, SPTBN1, SPTBN2, SPTLC3, SPTSSA, SPX, SQLE, SQRDL, SQSTM1, SRBD1, SRC, SRCAP, SRD5A1, SRD5A2, SREBF2, SREK1IP1, SRGAP1, SRGAP2, SRGAP2B, SRGN, SRI, SRP14-AS1, SRPK2, SRPX2, SRRD, SRRM2, SRRT, SRSF1, SRSF2, SRSF3, SRSF5, SRSF6, SRSF7, SRSF8, SRXN1, SSB, SSBP3, SSBP4, SSH1, SSH2, SSH3, SSR4, SSRP1, SSTR1, SSX2IP, ST14, ST20-AS1, ST3GAL2, ST3GAL3, ST3GAL4, ST3GAL5, ST5, ST6GAL1, ST6GALNAC2, ST6GALNAC4, ST7, STAC3, STAG1, STAG2, STAG3L5P-PVRIG2P-PILRB, STAM2, STAP2, STARD10, STARD13, STARD4, STARD5, STARD8, STAT1, STAT3, STAT4, STAT5A, STAT5B, STAT6, STAU2, STBD1, STC2, STEAP1, STEAP2, STEAP3, STIM1, STIP1, STK10, STK11IP, STK17A, STK3, STK36, STK38L, STMN1, STOML1, STON2, STPG1, STRADA, STRBP, STRIP1, STRN, STX17, STX18, STX1A, STX3, STX4, STX5, STX6, STX7, STX8, STXBP1, STXBP3, STXBP4, STXBP5, STYX, SUCLG2-AS1, SUDS3, SUFU, SUGT1, SULF1, SULF2, SUOX, SUPT16H, SUPT20H, SUPT3H, SUPT6H, SUPT7L, SURF2, SUSD6, SUV39H1, SUV420H1, SUV420H2, SUZ12, SUZ12P1, SVBP, SVIL, SVIP, SWSAP1, SWT1, SYDE1, SYDE2, SYNCRIP, SYNE1, SYNE3, SYNE4, SYNGR2, SYNJ2, SYNPO, SYNRG, SYPL2, SYT12, SYT17, SYT9, SZRD1, TAB1, TAB3, TACC1, TACC2, TACC3, TACO1, TACSTD2, TADA1, TADA2A, TAF11, TAF13, TAF1B, TAF2, TAF3, TAF4, TAF6L, TAF8, TAF9B, TAGLN, TAMM41, TANC1, TANGO6, TAOK1, TAOK2, TAOK3, TAP1, TAP2, TAPT1, TARBP1, TARS2, TASP1, TATDN3, TAX1BP1, TAX1BP3, TBC1D1, TBC1D10A, TBC1D12, TBC1D15, TBC1D16, TBC1D19, TBC1D2, TBC1D22A, TBC1D23, TBC1D24, TBC1D25, TBC1D31, TBC1D4, TBC1D5, TBC1D7, TBC1D8B, TBC1D9, TBC1D9B, TBCA, TBCC, TBCCD1, TBCD, TBCE, TBCEL, TBCK, TBL1X, TBL1XR1, TBPL1, TBX15, TBX19, TBX3, TCAF1, TCEA1, TCEA2, TCEAL1, TCEAL3, TCEAL8, TCEB2, TCERG1, TCF12, TCF3, TCF7, TCF7L1, TCF7L2, TCHP, TCIRG1, TCOF1, TCP11L2, TCTA, TCTEX1D2, TCTN3, TDP1, TDRD3, TDRD7, TDRKH, TEAD1, TEAD3, TEAD4, TECR, TEFM, TENM1, TEP1, TERF1, TESC, TET2, TEX10, TEX2, TEX30, TF, TFAP4, TFCP2, TFDP2, TFE3, TFEB, TFG, TFPI, TFR2, TFRC, TGFA, TGFB1, TGFB1I1, TGFB3, TGFBR1, TGFBR2, TGFBR3L, TGIF2-C20orf24, TGM1, TGM2, TGS1, THADA, THAP10, THAP2, THAP6, THAP7, THAP8, THAP9, THAP9-AS1, THEM6, THNSL1, THOC1, THOC6, THOP1, THPO, THRAP3, THRB, THSD1, THYN1, TIA1, TIAM1, TICAM1, TIFA, TIGAR, TIGD2, TIGD5, TIMELESS, TIMM10, TIMM8B, TIMM9, TIMP1, TIMP2, TINAGL1, TINF2, TIPIN, TIPRL, TIRAP, TJP1, TK1, TK2, TKT, TLCD1, TLCD2, TLDC1, TLE1, TLE4, TLK1, TLK2, TLN1, TLN2, TLR1, TLR2, TLR3, TLR4, TM4SF1, TM4SF4, TM4SF5, TM7SF2, TM9SF4, TMA7, TMC4, TMCC2, TMC03, TMC04, TMC06, TMED6, TMED7-TICAM2, TMED8, TMEM102, TMEM107, TMEM109, TMEM116, TMEM120B, TMEM121, TMEM128, TMEM129, TMEM131, TMEM132A, TMEM133, TMEM134, TMEM136, TMEM139, TMEM140, TMEM141, TMEM144, TMEM150C, TMEM154, TMEM156, TMEM158, TMEM159, TMEM160, TMEM161B, TMEM165, TMEM167B, TMEM168, TMEM170A, TMEM170B, TMEM176A, TMEM176B, TMEM177, TMEM184A, TMEM184C, TMEM185B, TMEM187, TMEM189, TMEM2, TMEM200B, TMEM201, TMEM220, TMEM223, TMEM233, TMEM238, TMEM243, TMEM246, TMEM256, TMEM256-PLSCR3, TMEM258, TMEM261, TMEM263, TMEM265, TMEM27, TMEM30B, TMEM38A, TMEM39B, TMEM41A, TMEM44, TMEM50A, TMEM52, TMEM53, TMEM54, TMEM56, TMEM57, TMEM63B, TMEM67, TMEM68, TMEM70, TMEM74B, TMEM79, TMEM82, TMEM87B, TMEM88, TMEM92, TMF1, TMLHE, TMOD1, TMOD2, TMPO, TMPRSS2, TMPRSS6, TMSB10, TMTC1, TMTC2, TMTC3, TMX4, TNFAIP8, TNFAIP8L1, TNFRSF10A, TNFRSF10B, TNFRSF10C, TNFRSF10D, TNFRSF14, TNFRSF19, TNFRSF1A, TNFRSF1B, TNFRSF25, TNFSF10, TNFSF12, TNFSF13B, TNFSF14, TNIK, TNIP1, TNIP2, TNK2, TNKS, TNKS1BP1, TNKS2, TNPO2, TNPO3, TNRC6B, TNRC6C, TNS1, TNS3, TOB1, TOB2, TOE1, TOM1L2, TOMM40, TOMM40L, TOMM7, TOP1MT, TOP2B, TOPBP1, TOR1B, TOR3A, TP53, TP53BP1, TP53BP2, TP53I13, TP53I3, TP53INP2, TP73-AS1, TPBG, TPCN1, TPCN2, TPD52, TPGS1, TPK1, TPM1, TPM2, TPM4, TPP2, TPR, TPRN, TPST1, TPT1, TPX2, TRAF1, TRAF3IP2, TRAF4, TRAFD1, TRAK1, TRAK2, TRAM1, TRAM2, TRANK1, TRAPPC10, TRAPPC11, TRAPPC12, TRAPPC13, TRAPPC2B, TRAPPC5, TRAPPC6A, TRAPPC6B, TRAPPC8, TRAPPC9, TREH, TRIAP1, TRIB1, TRIB2, TRIB3, TRIM13, TRIM15, TRIM16, TRIM16L, TRIM2, TRIM21, TRIM23, TRIM24, TRIM26, TRIM29, TRIM3, TRIM33, TRIM34, TRIM38, TRIM45, TRIM47, TRIMS, TRIM52, TRIM52-AS1, TRIM6, TRIM62, TRIM65, TRIM66, TRIM68, TRIM7, TRIMS, TRIO, TRIP11, TRIP12, TRIP13, TRIP4, TRIT1, TRMT10A, TRMT10B, TRMT11, TRMT112, TRMT12, TRMT13, TRMT1L, TRMT2A, TRMT61B, TRNP1, TRPM4, TRPT1, TRPV1, TRPV4, TRRAP, TSC22D1, TSC22D2, TSC22D4, TSEN15, TSEN2, TSEN54, TSHZ1, TSHZ2, TSLP, TSNARE1, TSPAN12, TSPAN13, TSPAN14, TSPAN15, TSPAN4, TSPAN6, TSPAN8, TSPAN9, TSPO, TSPO2, TSPYL1, TSPYL2, TSPYL4, TSR1, TSSC1, TSSC4, TSSK6, TSTD1, TSTD2, TSTD3, TTBK2, TTC21B, TTC23, TTC26, TTC27, TTC28, TTC31, TTC39B, TTC39C, TTC3P1, TTC5, TTC7B, TTC8, TTC9, TTF1, TTF2, TTI2, TTLL1, TTLL11, TTLL12, TTLL4, TTLL5, TTLL7, TTPA, TTPAL, TTR, TUBA1C, TUBB, TUBB2A, TUBB4B, TUBB6, TUBE1, TUBG2, TUBGCP3, TUBGCP4, TUBGCP5, TUBGCP6, TUG1, TULP3, TULP4, TUSC8, TWF2, TWISTNB, TWSG1, TXLNG, TXN, TXNDC11, TXNDC17, TXNDC9, TXNRD1, TXNRD3, TYK2, TYMP, TYMS, TYRO3, TYSND1, TYVV1, TYVV1B, U2AF2, U2SURP, UACA, UAP1, UBA1, UBA2, UBA3, UBA52, UBA6, UBA6-AS1, UBA7, UBAC2, UBALD2, UBAP1L, UBAP2, UBAP2L, UBASH3B, UBD, UBE2D1, UBE2D4, UBE2E2, UBE2G1, UBE2H, UBE2J1, UBE2K, UBE2MP1, UBE2R2, UBE2S, UBE2T, UBE3C, UBE3D, UBE4B, UBFD1, UBIAD1, UBL4A, UBL5, UBL7-AS1, UBN2, UBOX5, UBQLN1, UBQLN2, UBQLN4, UBR1, UBR2, UBR3, UBR5, UBR5-AS1, UBR7, UBTD1, UBTF, UBXN2A, UBXN2B, UCHL5, UCN, UFL1, UFSP1, UGDH, UGT1A1, UGT1A6, UGT2A3, UGT2B10, UGT2B15, UGT2B17, UGT2B28, UGT2B4, UGT2B7, UGT3A1, UHMK1, UHRF1BP1, UHRF1BP1L, UIMC1, ULBP3, ULK1, ULK4, UMAD1, UNC119, UNC13B, UNC13D, UNC5B, UNG, UNK, UPB1, UPP1, UPRT, UQCC3, UQCR10, UQCR11, UQCRB, UQCRH, UQCRHL, UQCRQ, URB1, URB1-AS1, URB2, URGCP, UROC1, USB1, USMG5, USP1, USP11, USP13, USP20, USP21, USP22, USP24, USP25, USP27X, USP28, USP30, USP32, USP33, USP34, USP35, USP43, USP46, USP47, USP49, USP5, USP53, USP54, USP6NL, USP8, USP9X, USPL1, UTP11L, UTP14A, UTP15, UTP18, UTP20, UTRN, UVRAG, UVSSA, VAC14, VAMP1, VAMP4, VAMPS, VANGL1, VARS, VASN, VASP, VAV2, VCL, VCP, VDAC1, VDR, VEGFA, VIL1, VIPAS39, VKORC1, VM01, VNN1, VNN2, VNN3, VPREB3, VPS13A, VPS13B, VPS13C, VPS13D, VPS18, VPS26B, VPS33A, VPS33B, VPS37A, VPS37D, VPS41, VPS50, VPS51, VPS52, VPS53, VPS54, VPS72, VPS8, VRK1, VRK2, VSIG10L, VSNL1, VTCN1, VTI1A, VTN, VWA1, VWA5A, VWA7, VWA8, WARS, WARS2, WASL, WBP1, WBP1L, WBSCR16, WBSCR27, WDFY2, WDFY3, WDR12, WDR18, WDR24, WDR25, WDR26, WDR35, WDR36, WDR37, WDR4, WDR44, WDR45, WDR48, WDR5, WDR55, WDR5B, WDR7, WDR70, WDR72, WDR75, WDR89, WDR91, WDR92, WEE1, WFDC2, WIBG, WIPF3, WIPI2, WIZ, WLS, WNK1, WNT3, WNT5A, WRN, WSB1, WSB2, WTAP, WWC1, WWC2, WWC3, WWOX, WWP1, WWP2, WWTR1, XBP1, XDH, XIST, XKR8, XPC, XPNPEP1, XPNPEP2, XPO4, XPO7, XPOT, XRCC4, XRCC5, XRCC6, XRCC6BP1, XRN1, XRRA1, XXYLT1, XYLT2, YAE1D1, YAF2, YAP1, YARS2, YBX1, YDJC, YEATS2, YEATS4, YJEFN3, YLPM1, YPEL3, YPEL5, YTHDC2, YTHDF3-AS1, YWHAG, ZADH2, ZAK, ZBED3, ZBED5, ZBED6, ZBED6CL, ZBED9, ZBTB14, ZBTB20, ZBTB21, ZBTB24, ZBTB26, ZBTB3, ZBTB33, ZBTB34, ZBTB38, ZBTB39, ZBTB40, ZBTB41, ZBTB42, ZBTB43, ZBTB44, ZBTB45, ZBTB7A, ZBTB7B, ZBTB8A, ZBTB9, ZC2HC1A, ZC3H10, ZC3H12A, ZC3H12C, ZC3H14, ZC3H3, ZC3H4, ZC3H7B, ZC3HC1, ZC4H2, ZCCHC10, ZCCHC11, ZCCHC2, ZCCHC24, ZCCHC4, ZDBF2, ZDHHC13, ZDHHC14, ZDHHC16, ZDHHC17, ZDHHC18, ZDHHC2, ZDHHC23, ZDHHC8, ZEB1-AS1, ZER1, ZFAND2A, ZFAND2B, ZFAND3, ZFAND4, ZFAS1, ZFAT, ZFHX3, ZFHX4, ZFP1, ZFP30, ZFP36, ZFP36L1, ZFP36L2, ZFP62, ZFP69, ZFP69B, ZFP90, ZFP91, ZFPM1, ZFYVE16, ZFYVE26, ZFYVE28, ZFYVE9, ZGLP1, ZHX2, ZHX3, ZIC1, ZKSCAN2, ZKSCAN3, ZKSCAN8, ZMAT1, ZMAT3, ZMAT5, ZMIZ1, ZMIZ2, ZMPSTE24, ZMYM1, ZMYM2, ZMYM3, ZMYM4, ZMYM6, ZMYND12, ZMYND15, ZNF10, ZNF100, ZNF106, ZNF107, ZNF112, ZNF117, ZNF121, ZNF131, ZNF133, ZNF134, ZNF141, ZNF142, ZNF146, ZNF148, ZNF155, ZNF165, ZNF17, ZNF175, ZNF18, ZNF180, ZNF181, ZNF182, ZNF184, ZNF189, ZNF195, ZNF20, ZNF205, ZNF211, ZNF212, ZNF217, ZNF222, ZNF224, ZNF23, ZNF232, ZNF235, ZNF236, ZNF239, ZNF248, ZNF25, ZNF253, ZNF254, ZNF260, ZNF267, ZNF274, ZNF275, ZNF280C, ZNF280D, ZNF281, ZNF282, ZNF284, ZNF286A, ZNF292, ZNF296, ZNF30, ZNF302, ZNF318, ZNF319, ZNF32, ZNF324, ZNF324B, ZNF326, ZNF334, ZNF335, ZNF337, ZNF33A, ZNF34, ZNF341, ZNF354A, ZNF362, ZNF367, ZNF37A, ZNF383, ZNF385A, ZNF385B, ZNF395, ZNF407, ZNF41, ZNF414, ZNF416, ZNF419, ZNF429, ZNF432, ZNF433, ZNF436, ZNF440, ZNF441, ZNF444, ZNF445, ZNF449, ZNF45, ZNF451, ZNF460, ZNF468, ZNF473, ZNF48, ZNF480, ZNF484, ZNF485, ZNF490, ZNF493, ZNF503, ZNF503-AS2, ZNF506, ZNF512, ZNF512B, ZNF516, ZNF518A, ZNF524, ZNF542P, ZNF550, ZNF552, ZNF559, ZNF561, ZNF563, ZNF569, ZNF579, ZNF580, ZNF581, ZNF582-AS1, ZNF583, ZNF585A, ZNF586, ZNF587B, ZNF593, ZNF596, ZNF608, ZNF609, ZNF614, ZNF615, ZNF618, ZNF619, ZNF620, ZNF621, ZNF627, ZNF628, ZNF638, ZNF639, ZNF641, ZNF652, ZNF654, ZNF662, ZNF668, ZNF672, ZNF675, ZNF680, ZNF684, ZNF688, ZNF689, ZNF69, ZNF691, ZNF692, ZNF697, ZNF700, ZNF703, ZNF704, ZNF707, ZNF708, ZNF710, ZNF74, ZNF746, ZNF747, ZNF764, ZNF77, ZNF770, ZNF771, ZNF777, ZNF778, ZNF780A, ZNF780B, ZNF784, ZNF785, ZNF786, ZNF787, ZNF789, ZNF79, ZNF81, ZNF821, ZNF839, ZNF84, ZNF841, ZNF844, ZNF852, ZNF862, ZNF92, ZNHIT2, ZNHIT6, ZNRD1, ZNRF1, ZNRF2, ZNRF3, ZP3, ZRSR2, ZSCAN12, ZSCAN16-AS1, ZSCAN18, ZSCAN20, ZSCAN26, ZSCAN29, ZSCAN31, ZSCAN9, ZSWIM1, ZSWIM3, ZSWIM4, ZSWIM6, ZSWIM8, ZW10, ZWILCH, ZWINT, ZXDC, ZYG11B, ZYX, ZZEF1, ZZZ3, CCDC109B, COL12A1, CXCL9, FUT8, GBP3, GHRL, GOLT1A, HMGA1, INSC, NINJ2, OXER1, TIMP3, TTC39A, AADAT, AASDH, ABCA2, ABCA3, ACAD10, ACCS, ACIN1, ACP2, ACP6, ACSL4, ACSS3, ADAM17, ADRM1, AHCTF1, AIFM2, AKAP8L, AKIRIN2, AKR7L, ALDOC, ALG6, ALKBH5, ALS2CL, AMMECR1L, ANKHD1-EIF4EBP3, ANKRD10, ANKRD6, AP5Z1, APH1B, APITD1, AQP11, ARF1, ARF4, ARHGAP21, ARHGAP33, ARHGEF10, ARHGEF37, ARHGEF6, ARHGEF9, ARID1A, ARID3B, ARPIN, ASB6, ASGR1, ATP6VOC, ATP6V1B2, ATP8B3, ATP8B4, ATXN1L, AUH, B3GNT5, B4GALT1, BAG1, BCKDHA, BEND7, BRD1, BRD2, BRPF1, BTN2A1, BUD31, C11orf80, C12orf49, C12orf66, C14orf132, C16orf72, C1orf52, C20orf24, C2orf76, C4orf33, C4orf36, CAMSAP3, CAPS2, CASP4, CASP8, CATSPER2, CBLL1, CBY1, CC2D2A, CCDC122, CCDC125, CCDC142, CCDC57, CCDC68, CCDC71, CCDC9, CCDC90B, CCDC93, CCL28, CD40, CD82, CDK10, CDK5RAP3, CDKN2AIP, CDNF, CDR2, CENPL, CENPN, CENPQ, CEP41, CFAP20, CHAF1B, CHEK1, CHKA, CHTF18, CLTA, CMC4, CNNM4, CNOT4, CNTFR, COX19, CPB2-AS1, CPN1, CPT1B, CROP, CREM, CRKL, CRTC2, CSNK1A1L, CSRNP1, CTNNAL1, CWC22, CWC25, CXorf38, CYP2B7P, CYP4V2, D2HGDH, DCP1B, DCTN5, DDIT3, DENND5A, DET1, DHFRL1, DHX32, DHX58, DLGAP1-AS1, DNAH5, DNAJB6, DNAJC16, DNAJC25, DNAL1, DNAL4, DNASE1L1, DOCK10, DOCK6, DPP4, DRAM1, DTX2, DUSP14, DUT, DVL2, DYNLL2, DYRK1A, E2F6, EAF1, ECHDC3, EFHC1, EFTUD1, EIF1, EIF1AD, EIF1B, EIF4A3, EIF5, ELL2, ELMO3, ELMOD2, ELP4, ENKUR, EPCAM, EPHX2, EPN2, ERMAP, ERV3-1, ETF1, EXD3, EXOC1, EZH1, F11, FAM104B, FAM120B, FAM73A, FAM76A, FBXO11, FBXO30, FBXO42, FERMT1, FERMT2, FGF14-AS2, FHL2, FLCN, FLT3LG, FNBP4, FOSL2, FOXK2, FOXO3, FOXP1, FRS2, FUT4, FXR2, FZD6, GABPA, GABPB1, GAMT, GAPDH, GAPVD1, GAS6-AS1, GAS8, GATS, GATSL3, GBAS, GBP1, GCA, GCC1, GCC2, GCSHP3, GDNF-AS1, GET4, GFER, GINS1, GK, GLUL, GMDS-AS1, GMEB1, GNMT, GOLGA1, GOLGA2P5, GOLGA8A, GPALPP1, GPATCH11, GPATCH3, GPBP1, GPI, GRAMD4, GRIA3, GRPEL1, GSR, GSTA4, GTF2E1, GTPBP10, GUSBP4, GZF1, HAUS5, HEIH, HEXDC, HFE, HIAT1, HIVEP2, HLA-C, HLA-F, HLA-J, HLA-L, HMGXB4, HNRNPUL2, HOMER1, HSF4, HSP90AB1, HSPA14, HSPE1, HYI, ICA1, IL18R1, INTS2, IP6K1, IST1, ITPKC, ITPRIP, JMJD6, JRKL, KATNAL1, KCMF1, KCTD6, KDM2A, KIAA0907, KIAA1143, KLHL11, KLHL2, KLHL5, KRCC1, KTI12, LATS2, LCLAT1, LEPR, LGALS3, LGALS8, LIMK2, LINC00174, LINC00341, LINC01128, LINC01503, LOC100499489, LOC100507547, LOC100507577, LOC101927630, LOC101927843, LOC103021295, LOC155060, LRRC23, LRRC39, LRRC58, LSM8, LUC7L3, LY75, LZTFL1, MAGOH, MAL2, MAML1, MAN2C1, MAPK6, MAPK7, MAPRE1, MCM2, MECP2, MED10, MED13, MED26, MEF2D, METTL3, MEX3C, MGAT1, MICALL1, MIEF1, MMP25-AS1, MOB3B, MOCOS, MOGAT3, MORC3, MORF4L1, MOSPD1, MPI, MPPS, MSL2, MTF1, MTHFS, MUT, MXI1, MYPN, NAA30, NAA60, NCOA6, NDEL1, NEMP1, NEU4, NHLRC3, NICN1, NIPBL, NME7, NMRK1, NNT-AS1, NOL3, NRF1, NRSN2-AS1, NT5DC3, NUP153, NUTM2B-AS1, OAF, ODC1, OSCP1, OSMR, P4HA2, PAN2, PAPD7, PARP3, PBLD, PCF11, PCGF2, PCMTD2, PDRG1, PDSS1, PELO, PEX1, PF4V1, PFKFB3, PFKL, PGK1, PHF1, PHF12, PHF13, PHYHD1, PI4K2A, PICK1, PIGB, PIM3, PIPDX, PITHD1, PITPNM1, PKN3, PKNOX1, PLA2G12A, PLAGL2, PLSCR1, PMEL, POM121, POM121C, POU2F1, PPAT, PPIC, PPDX, PPP1R13L, PPP1R3E, PPP2CA, PPP2CB, PPP2R2A, PPP3CB-AS1, PPP3R1, PPP4R3A, PRCC, PRDM10, PRDM2, PRKRIR, PRPF40B, PRR3, PRSS53, PSMD12, PSMD6-A52, PSMG3-AS1, PTPN12, PXN-AS1, QTRT1, R3HCC1L, RAB21, RAB38, RAB3IP, RAB7A, RAB8B, RABL2B, RAD9A, RASGRP3, RASSF1, RASSF4, RBAK, RBBP6, RBL2, RBM14-RBM4, RBM39, RC3H1, RGMB, RGN, RHEB, RIT1, RND3, RNF10, RNF114, RNF144A, RNF168, RNF19B, RNF215, RNF219, RNF24, RNF4, RRAGC, RSRC2, RYBP, S100PBP, SAFB2, SCAF4, SCAND2P, SCML1, SCRN3, SDC3, SDE2, SDF2, SDHAF1, SENP8, SEPT7P2, SERP1, SERP2, SERPINB8, SERTAD3, SETD1B, SETD8, SFI1, SGTB, SIAH2, SIRT3, SIRT7, SKI, SKIV2L, SLC15A3, SLC16A1, SLC22A7, SLC23A1, SLC29A4, SLC2A11, SLC30A4, SLC35E2, SLC35E2B, SLC3A1, SLMO2-ATP5E, SMC6, SMCR8, SNHG3, SNRNP48, SNX11, SNX16, SON, SORBS3, SPAG5, SPAG9, SPATA18, SPATA2, SPATA20, SPATA24, SPG20, SPPL2B, SPTBN5, SPTLC2, SPTY2D1, SRF, SS18, ST3GAL1, STARD7, STK32C, STK35, STRN4, STS, STX11, SUCO, SURF4, SVIL-AS1, SYBU, SYNGAP1, TADA2B, TBC1D20, TBC1D2B, TBKBP1, TCEA3, TCEB1, TCTN1, TDP2, TECPR2, TERF2IP, TESK1, TFB1M, TFDP1, THAP11, THAP3, THBS3, THSD4, TLE3, TMEM11, TMEM115, TMEM117, TMEM171, TMEM175, TMEM181, TMEM198B, TMEM231, TMEM260, TMEM47, TMEM80, TMEM81, TMEM91, TMPRSS9, TMTC4, TNFAIP1, TNFRSF11B, TNPO1, TOP1, TOR1AIP1, TP53TG1, TPI1P2, TPRA1, TPT1-AS1, TRAPPC2, TREX1, TRIM10, TRIM25, TRIM28, TRIM41, TRIOBP, TRIP10, TRMT5, TRUB1, TTC12, TTC13, TTC22, TTN-AS1, TUBA4B, TXNL1, TXNL4B, UBAP1, UBN1, UNKL, UPF1, USP12, USP30-AS1, USP36, USP38, USP7, UXS1, VCPIP1, VMAC, VPS37B, VPS9D1, WAC, WDR20, WDR27, WDR31, WDR45B, WDR47, WDR60, WDR78, WHAMM, WHSC1, XRCC1, YRDC, YTHDF1, YTHDF2, YTHDF3, YY1, YY1AP1, YY2, ZCCHC3, ZCCHC8, ZDHHC21, ZFAND5, ZFYVE21, ZMYM5, ZMYND19, ZNF143, ZNF160, ZNF207, ZNF252P, ZNF263, ZNF354B, ZNF37BP, ZNF382, ZNF397, ZNF410, ZNF420, ZNF438, ZNF44, ZNF514, ZNF528, ZNF544, ZNF558, ZNF561-AS1, ZNF567, ZNF574, ZNF622, ZNF623, ZNF800, ZNF823, ZNF83, ZNF846, ZSCAN16, and ZSCAN30 were classified as dynamic. Group 3 of dynamic genes was observed to include 71 disease-associated genes.

A1BG-AS1, A2M-AS1, A2ML1, A2MP1, A3GALT2, A4GNT, M06, AACSP1, AADACL2, AADACL2-AS1, AADACL3, AADACL4, AANAT, MRD, AATK, AATK-AS1, ABCA10, ABCA13, ABCA17P, ABCA4, ABCA9, ABCA9-AS1, ABCB5, ABCC11, ABCC12, ABCC13, ABCC5-AS1, ABCC8, ABCD2, ABCG4, ABHD1, ABHD11-AS1, ABHD12B, ABI3, ABI3BP, ABO, ABRA, ACAN, ACBD7, ACCSL, ACE, ACER1, ACKR1, ACKR4, ACOT11, ACOXL, ACPP, ACPT, ACR, ACRV1, ACSBG1, ACSBG2, ACSM4, ACSM6, ACTA2-AS1, ACTC1, ACTG1P17, ACTG1P4, ACTL6B, ACTL7A, ACTL7B, ACTL8, ACTL9, ACTN1-AS1, ACTN2, ACTN3, ACTR3BP2, ACTR3BP5, ACTRT1, ACTRT2, ACVR1C, ACVRL1, ADAD1, ADAD2, ADAM11, ADAM12, ADAM18, ADAM2, ADAM20, ADAM20P1, ADAM21, ADAM21P1, ADAM22, ADAM23, ADAM28, ADAM29, ADAM30, ADAM32, ADAM33, ADAM3A, ADAM5, ADAM6, ADAM7, ADAMDEC1, ADAMTS12, ADAMTS14, ADAMTS15, ADAMTS16, ADAMTS18, ADAMTS19, ADAMTS19-AS1, ADAMTS2, ADAMTS20, ADAMTS3, ADAMTS4, ADAMTS5, ADAMTS6, ADAMTS7P1, ADAMTS8, ADAMTS9-AS1, ADAMTS9-AS2, ADAMTSL1, ADAMTSL2, ADAP1, ADAP2, ADARB2, ADARB2-AS1, ADCY10, ADCY2, ADCY3, ADCY4, ADCY8, ADCYAP1, ADCYAP1R1, ADD2, ADD3-AS1, ADGB, ADGRA1, ADGRA1-AS1, ADGRA2, ADGRB1, ADGRB2, ADGRB3, ADGRE1, ADGRE2, ADGRE3, ADGRE4P, ADGRF1, ADGRF2, ADGRF5, ADGRG2, ADGRL3, ADGRG4, ADGRG5, ADGRL1, ADGRL3, ADGRL3-AS1, ADGRL4, ADGRV1, ADH7, ADIG, ADIPOQ, ADIPOQ-AS1, ADORA1, ADORA2B, ADORA3, ADPGK-AS1, ADRA1D, ADRA2B, ADRB1, ADRB3, ADRBK2, ADTRP, AEBP1, AFAP1-AS1, AFF2, AGAP11, AGAP2, AGAP2-AS1, AGAP4, AGAP7P, AGBL1, AGBL1-AS1, AGBL2, AGBL4, AGBL4-IT1, AGPAT4, AGPAT4-IT1, AGR2, AGR3, AGRP, AGTR2, AHRR, AHSP, AICDA, AIF1, AIF1L, AIFM3, AIM2, AIPL1, ARE, AIRN, AJAP1, AKS, AK7, AK8, AKAP14, AKAP4, AKAP5, AKNAD1, AKR1E2, AKR7A2P1, ALAS2, ALDH1L2, ALDH3B2, ALG1L2, ALG1L9P, ALK, ALKBH3-AS1, ALMS1-IT1, ALMS1P, ALOX12B, ALOX15, ALOX15B, ALOX15P1, ALOX5AP, ALPI, ALPP, ALPPL2, ALS2CR11, ALS2CR12, ALX1, ALX4, AMBN, AMELX, AMELY, AMER2, AMER3, AMICA1, AMPH, AMTN, AMY1B, AMY1C, AMY2A, AMZ1, ANAPC1P1, ANGPT1, ANGPT2, ANGPT4, ANGPTL5, ANHX, ANK1, ANK2, ANK3, ANKDD1A, ANKDD1B, ANKFN1, ANKK1, ANKLE1, ANKRD18B, ANKRD18DP, ANKRD19P, ANKRD20A11P, ANKRD20A12P, ANKRD20A19P, ANKRD20A3, ANKRD20A4, ANKRD20A5P, ANKRD20A8P, ANKRD20A9P, ANKRD26P1, ANKRD26P3, ANKRD30A, ANKRD30B, ANKRD3OBP2, ANKRD3OBP3, ANKRD31, ANKRD34A, ANKRD34B, ANKRD34C, ANKRD34C-AS1, ANKRD36B, ANKRD36BP2, ANKRD44-IT1, ANKRD45, ANKRD55, ANKRD60, ANKRD62, ANKRD62P1-PARP4P3, ANKRD63, ANKRD65, ANKRD66, ANKRD7, ANKS1B, ANKUB1, ANLN, ANO1-AS2, ANO2, ANO3, ANO4, ANO7, ANO9, ANOS1, ANP32A-IT1, ANP32D, ANTXRL, ANTXRLP1, ANXA8, AOAH, AOAH-IT1, AOC1, AP1B1P1, AP1M2, AP3B2, APBA2, APC2, APCDD1, APCDD1L, APCDD1L-AS1, APELA, APLNR, APOBEC1, APOBEC2, APOBEC3A, APOBEC3A_B, APOBEC3B-AS1, APOBEC3D, APOBEC4, APOBR, APOD, APOL5, APOOP5, AQP1, AQP10, AQP12A, AQP12B, AQP2, AQP4-AS1, AQP5, AQP6, AQP8, AREG, ARGFX, ARGFXP2, ARHGAP11A, ARHGAP11B, ARHGAP15, ARHGAP19-SLIT1, ARHGAP23, ARHGAP25, ARHGAP26-AS1, ARHGAP26-IT1, ARHGAP28, ARHGAP31, ARHGAP31-AS1, ARHGAP36, ARHGAP4, ARHGAP40, ARHGAP44, ARHGAP6, ARHGAP8, ARHGAP9, ARHGDIG, ARHGEF15, ARHGEF19, ARHGEF25, ARHGEF26-AS1, ARHGEF3-AS1, ARHGEF33, ARHGEF34P, ARHGEF38, ARHGEF38-IT1, ARHGEF4, ARHGEF7-AS1, ARHGEF7-AS2, ARHGEF9-IT1, ARL11, ARL13A, ARL14EPL, ARL17A, ARL17B, ARL2-SNX15, ARL5C, ARL9, ARMC2, ARMC2-AS1, ARMC3, ARMC4, ARMS2, ARNT2, ARNTL2-AS1, ARPP21, ARR3, ARRDC3-AS1, ARRDC5, ARSI, ART1, ART3, ARTN, ARX, ASAH2, ASAH2B, ASAP1-IT1, ASAP1-1T2, ASB10, ASB11, ASB12, ASB15, ASB16, ASB17, ASB18, ASB2, ASB4, ASB5, ASB9P1, ASCL2, ASCL3, ASCL4, ASCL5, ASIC1, ASIC2, ASIC4, ASIC5, ASMT, ASPHD1, ASPHD2, ASPM, ASPN, ASTL, ASTN1, ASTN2-AS1, ASXL3, ASZ1, ATAD3C, ATAD5, ATCAY, ATE1-AS1, ATG9B, ATL1, ATOH1, ATOH7, ATP10A, ATP10B, ATP11A-AS1, ATP11AUN, ATP12A, ATP13A4, ATP13A4-AS1, ATP13A5, ATP13A5-AS1, ATP1A2, ATP1A3, ATP1A4, ATP1B2, ATP1B4, ATP2A1-AS1, ATP2A3, ATP2B2-IT2, ATP2B3, ATP2C2, ATP4A, ATP4B, ATP5L2, ATP6V0A4, ATP6V0CP3, ATP6V0E2-AS1, ATP6V1B1, ATP6V1B1-AS1, ATP6V1C2, ATP6V1G2, ATP6V1G3, ATP8A1, ATP8A2, ATP8B5P, ATRNL1, ATXN3L, ATXN8OS, AUNIP, AURKB, AVPR1B, AVPR2, AWAT1, AWAT2, AXDND1, B3GALT1, B3GALT2, B3GALT4, B3GALT5, B3GALT5-AS1, B3GAT2, B3GNT4, B3GNT6, B3GNT7, B3GNT8, B4GALNT2, B4GALNT4, B4GALT4-AS1, BAALC, BAALC-AS1, BAALC-AS2, BACH1-IT2, BAGE3, BAGE4, BAGE5, BANCR, BANF2, BANK1, BARHL1, BARHL2, BARX1, BARX2, BASP1, BASP1P1, BAZ2B, BBOX1-AS1, BCAN, BCAT1, BCL11A, BCL11B, BCL2L14, BCL2L15, BCL6B, BCORP1, BCRP2, BCRP3, BDKRB1, BDNF, BEAN1, BECN2, BEGAIN, BEND2, BEND3P3, BEND4, BENDS, BEND6, BEST2, BEST3, BEST4, BFSP2, BGLT3, BGN, BHLHA9, BHLHE22, BHLHE23, BHLHE40-AS1, BHMG1, BIN2, BIN3-IT1, BIROS, BIRC6-AS2, BIRC7, BIRC8, BLACAT1, BLACE, BLID, BLK, BLM, BMP10, BMP15, BMP3, BMP5, BMP6, BMP7, BMP7-AS1, BMPER, BMPR1B, BMPR1B-AS1, BMS1P17, BMS1P18, BMS1P6, BMX, BNC1, BNC2, BNIPL, BOC, BOD1L2, BOK-AS1, BOLA2, BOLA3-AS1, BOLL, BPESC1, BPI, BPIFA1, BPIFA2, BPIFA3, BPIFA4P, BPIFB1, BPIFB2, BPIFB3, BPIFB4, BPIFB6, BPIFC, BPY2B, BPY2C, BRCA2, BRCAT107, BRCAT54, BRD7P3, BRDT, BRDTP1, BREA2, BRINP1, BRINP3, BRIP1, BRS3, BRSK1, BRSK2, BRWD1-AS1, BRWD1-IT2, BSN, BSN-AS2, BSND, BSPH1, BSX, BTBD17, BTBD18, BTF3P11, BTG4, BTK, BTLA, BTN1A1, BTNL10, BTNL2, BTNL3, BTNL8, BTNL9, BUB1, BVES, BVES-AS1, BZRAP1, BZRAP1-AS1, C10orf105, C10orf107, C10orf113, C10orf120, C10orf126, C10orf128, C10orf131, C10orf142, C10orf53, C10orf55, C10orf62, C10orf67, C10orf71, C10orf71-AS1, C10orf82, C10orf90, C10orf91, C10orf95, C10orf99, C11orf16, C11orf21, C11orf39, C11orf40, C11orf42, C11orf44, C11orf45, C11orf53, C11orf63, C11orf70, C11orf72, C11orf85, C11orf86, C11orf87, C11orf88, C11orf97, C12orf40, C12orf42, C12orf50, C12orf54, C12orf56, C12orf71, C12orf74, C12orf77, C12orf79, C12orf80, C14orf177, C14orf180, C14orf39, C15orf26, C15orf27, C15orf32, C15orf43, C15orf53, C15orf54, C15orf56, C15orf59, C15orf59-AS1, C16orf47, C16orf54, C16orf71, C16orf78, C16orf82, C16orf89, C16orf90, C16orf92, C16orf96, C16orf97, C17orf102, C17orf104, C17orf105, C17orf112, C17orf47, C17orf50, C17orf53, C17orf64, C17orf74, C17orf77, C17orf78, C17orf98, C17orf99, C18orf42, C18orf54, C18orf61, C18orf63, C19orf35, C19orf45, C19orf57, C19orf67, C19orf81, C19orf84, C1QA, C1QL2, C1QL4, C1QTNF2, C1QTNF7, C1QTNF8, C1QTNF9, C1QTNF9B, C1orf100, C1orf101, C1orf110, C1orf127, C1orf137, C1orf140, C1orf141, C1orf145, C1orf146, C1orf158, C1orf167, C1orf185, C1orf186, C1orf189, C1orf194, C1orf195, C1orf204, C1orf210, C1orf229, C1orf233, C1orf61, C1orf64, C1orf68, C1orf87, C1orf94, C1orf95, C20orf141, C20orf144, C20orf166-AS1, C20orf173, C20orf195, C20orf197, C20orf202, C20orf203, C20orf62, C20orf78, C20orf85, C21orf140, C21orf62, C21orf62-AS1, C21orf91-OT1, C22orf15, C22orf24, 022orf31, C22orf34, C22orf42, C2CD4B, C2CD4C, C2CD4D, C2orf27A, C2orf27B, C2orf40, C2orf48, C2orf50, C2orf57, C2orf61, C2orf66, C2orf70, C2orf71, C2orf73, C2orf78, C2orf80, C2orf81, C2orf83, C2orf91, C3AR1, C3orf20, C3orf22, C3orf30, C3orf36, C3orf49, C3orf56, C3orf67-AS1, C3orf70, C3orf79, C3orf80, C3orf84, C4orf17, C4orf22, C4orf45, C4orf51, C5AR1, C5orf17, C5orf34, C5orf38, C5orf47, C5orf49, C5orf58, C5orf60, C5orf64, C5orf66-AS1, C5orf66-AS2, C5orf67, C6orf10, C6orf118, C6orf132, C6orf15, C6orf163, C6orf165, C6orf222, C6orf223, C6orf229, C6orf58, C6orf7, C6orf99, C7, C7orf33, C7orf34, C7orf57, C7orf62, C7orf65, C7orf66, C7orf69, C7orf71, C7orf72, 07orf76, C7orf77, C8orf22, C8orf31, C8orf34, C8orf34-AS1, C8orf37-AS1, C8orf49, C8orf74, 08orf86, C8orf87, C8orf88, C9orf106, C9orf129, C9orf131, C9orf135, C9orf135-AS1, C9orf139, C9orf163, C9orf170, C9orf171, C9orf173, C9orf173-AS1, C9orf41-AS1, C9orf47, C9orf50, C9orf62, C9orf66, C9orf84, CA1, CA10, CA11, CA14, CA4, CASB, CA6, CA7, CA8, CABP1, CABP2, CABP4, CABPS, CABP7, CABS1, CACNA1A, CACNA1B, CACNA1C, CACNA1C-AS1, CACNA1C-AS2, CACNA1C-AS4, CACNA1C-IT2, CACNA1C-IT3, CACNA1E, CACNA1F, CACNA1G, CACNA1G-AS1, CACNA1I, CACNA1S, CACNA2D1, CACNA2D2, CACNA2D3, CACNA2D3-AS1, CACNA2D4, CACNB1, CACNB2, CACNB4, CACNG1, CACNG2, CACNG3, CACNG4, CACNG5, CACNG6, CACNG7, CACNG8, CACTIN-AS1, CADM2, CADM2-AS2, CADM3, CADM3-AS1, CADPS, CAGE1, CALB1, CALB2, CALCR, CALCRL, CALHM1, CALHM2, CALHM3, CALML3, CALML3-AS1, CALMLS, CALN1, CALR3, CALY, CAMK2A, CAMK4, CAMKK1, CAMKV, CAMP, CAND1.11, CAND2, CAPN11, CAPN14, CAPN6, CAPN9, CAPNS2, CAPSL, CAPZA3, CARD11, CARD14, CARD17, CARD18, CARD9, CARTPT, CASC1, CASC11, CASC15, CASC16, CASC17, CASC18, CASC2, CASC20, CASC21, CASC23, CASCS, CASC6, CASC8, CASC9, CASKIN1, CASP12, CASP14, CASP1P2, CASP5, CASQ1, CASQ2, CASR, CATIP, CATIP-AS2, CATSPER1, CATSPER2P1, CATSPER4, CATSPERB, CATSPERD, CAV3, CBLN1, CBLN2, CBLN4, CBWD3, CBWDS, CBX2, CBX3P2, CBY3, CC2D2B, CCAT2, CCBE1, CCDC102B, CCDC105, CCDC108, CCDC114, CCDC116, CCDC129, CCDC13-AS1, CCDC136, CCDC140, CCDC141, CCDC144A, CCDC144B, CCDC144CP, CCDC144NL, CCDC144NL-AS1, CCDC148, CCDC148-AS1, CCDC15, CCDC151, CCDC153, CCDC154, CCDC155, CCDC160, CCDC162P, CCDC166, CCDC168, CCDC169, CCDC169-SOHLH2, CCDC170, CCDC171, CCDC172, CCDC173, CCDC175, CCDC177, CCDC178, CCDC179, CCDC180, CCDC181, CCDC182, CCDC184, CCDC185, CCDC26, CCDC27, CCDC30, CCDC33, CCDC36, CCDC37, CCDC37-AS1, CCDC38, CCDC39, CCDC40, CCDC42, CCDC42B, CCDC54, CCDC60, CCDC63, CCDC64B, CCDC65, CCDC67, CCDC70, CCDC73, CCDC74A, CCDC74B, CCDC78, CCDC79, CCDC8, CCDC80, CCDC81, CCDC83, CCDC85A, CCDC87, CCDC88C, CCDC89, CCER1, CCER2, CCIN, CCKAR, CCKBR, CCL1, CCL11, CCL13, CCL17, CCL18, CCL19, CCL22, CCL23, CCL24, CCL25, CCL27, CCL3L3, CCL4L1, CCL4L2, CCM2L, CCNA1, CCNB2, CCNB3, CCND2, CCND2-AS1, CCNI2, CCNJL, CCNYL2, CCR1, CCR10, CCR2, CCR3, CCR4, CCR5, CCR6, CCR7, CCR8, CCR9, CCRL2, CCSER1, CCT6P3, CCT8L2, CD101, CD163L1, CD164L2, CD180, CD19, CD1A, CD1B, CD1C, CD1E, CD2, CD200, CD200R1, CD200R1L, CD207, CD209, CD22, CD226, CD244, CD247, CD248, CD27, CD28, CD300A, CD300C, CD300E, CD300LD, CD300LF, CD300LG, CD33, CD34, CD36, CD37, CD3E, CD3G, CD40LG, CD48, CD5, CD5L, CD6, CD69, CD7, CD70, CD72, CD79B, CD80, CD81-AS1, CD84, CD86, CD8A, CD8B, CD93, CD96, CDC14C, CDC20B, CDC25A, CDC42EP5, CDC42P3, CDCA3, CDCA7, CDCP1, CDCP2, CDH10, CDH11, CDH12, CDH13, CDH15, CDH16, CDH17, CDH18, CDH19, CDH2O, CDH22, CDH23, CDH26, CDH3, CDH4, CDH5, CDH7, CDH8, CDH9, CDHR1, CDHR4, CDIPT-AS1, CDK1, CDK15, CDK5R2, CDKL2, CDKL4, CDKN2A-AS1, CDKN2B-AS1, CDR1, CDRT1, CDRT15, CDRT15L2, CDRT15P1, CDRT15P2, CDRT7, CDRT8, CDS1, CDSN, CDT1, CDX1, CDX2, CDX4, CDY1, CDY1B, CDY2B, CEACAM18, CEACAM20, CEACAM21, CEACAM22P, CEACAM3, CEACAM4, CEACAM5, CEACAM7, CEACAM8, CEBPE, CECR1, CECR3, CECR6, CECR7, CEL, CELA1, CELA2A, CELA2B, CELA3A, CELA3B, CELF2, CELF2-AS1, CELF2-AS2, CELF3, CELF4, CELF5, CELP, CELSR3, CEMIP, CEND1, CENPE, CENPF, CENPI, CENPM, CENPP, CENPVP1, CENPVP2, CEP126, CEP128, CEP170P1, CEP295NL, CEP55, CEP83-AS1, CER1, CERKL, CERS1, CERS3, CERS3-AS1, CERS6-AS1, CES1P2, CES5A, CES5AP1, CETN1, CETP, CFAP43, CFAP44-AS1, CFAP45, CFAP46, CFAP47, CFAP52, CFAP54, CFAP58, CFAP58-AS1, CFAP61, CFAP74, CFAP99, CFC1B, CFD, CFL1P1, CFP, CFTR, CGA, CGB, CGB1, CGB2, CGB5, CGB8, CH17-408M7.1, CH25H, CHAT, CHD3, CHD5, CHEK2P2, CHGA, CHGB, CHIA, CHIAP2, CHIT1, CHKB-CPT1B, CHL1, CHL1-AS1, CHMP1B2P, CHODL, CHODL-AS1, CHP2, CHRDL1, CHRFAM7A, CHRM1, CHRM2, CHRM3-AS1, CHRM3-AS2, CHRM4, CHRM5, CHRNA1, CHRNA2, CHRNA3, CHRNA4, CHRNA5, CHRNA6, CHRNA7, CHRNA9, CHRNB2, CHRNB3, CHRNB4, CHRND, CHRNG, CHST10, CHST2, CHST3, CHST4, CHST5, CHST6, CHST8, CHSY3, CIB3, CIB4, CIDEA, CIITA, CILP, CILP2, CISTR, CIT, CKAP2L, CKLF-CMTM1, CKMT1A, CKMT1B, CKMT2, CLC, CLCA1, CLCA2, CLCA3P, CLCA4, CLCN1, CLCNKA, CLCNKB, CLDN10, CLDN10-AS1, CLDN11, CLDN17, CLDN18, CLDN19, CLDN20, CLDN22, CLDN24, CLDN25, CLDN34, CLDN5, CLDN8, CLEC10A, CLEC12A, CLEC12B, CLEC14A, CLEC17A, CLEC18A, CLEC18B, CLEC18C, CLEC19A, CLEC1A, CLEC1B, CLEC2A, CLEC2D, CLEC2L, CLEC3A, CLEC4C, CLEC4D, CLEC4E, CLEC4F, CLEC4G, CLEC4GP1, CLEC4M, CLEC5A, CLEC6A, CLEC7A, CLEC9A, CLECL1, CLHC1, CLIC2, CLIC3, CLIC5, CLIC6, CLLU1, CLLU1OS, CLMP, CLNK, CLPS, CLPSL1, CLPSL2, CLRN1, CLRN1-AS1, CLRN2, CLSPN, CLSTN2, CLSTN2-AS1, CLUL1, CLVS1, CLVS2, CLYBL-AS1, CLYBL-AS2, CMA1, CMAHP, CMKLR1, CMTM2, CMTM5, CNBD1, CNBD2, CNFN, CNGA2, CNGA3, CNGA4, CNGB1, CNGB3, CNIH2, CNIH3, CNKSR2, CNPY1, CNR1, CNR2, CNRIP1, CNTD1, CNTFR-AS1, CNTN1, CNTN2, CNTN4, CNTN4-AS1, CNTN4-AS2, CNTN5, CNTN6, CNTNAP3, CNTNAP3B, CNTNAP3P2, CNTNAP4, CNTNAP5, COCH, COL10A1, COL11A1, COL13A1, COL14A1, COL15A1, COL18A1-AS1, COL18A1-AS2, COL19A1, COL1A2, COL20A1, COL22A1, COL23A1, COL24A1, COL25A1, COL26A1, COL28A1, COL2A1, COL3A1, COL4A1, COL4A2, COL4A2-AS1, COL4A3, COL4A4, COL4A5, COL4A6, COL5A1, COL6A3, COL6A4P1, COL6A4P2, COL6A5, COL6A6, COL8A1, COL8A2, COL9A1, COL9A2, COLCA1, COLEC10, COLEC12, COLGALT2, COMP, CORIN, CORO2B, COR06, CORT, COX412, COX6B2, COX7B2, COX8C, CPA1, CPA2, CPA3, CPA5, CPA6, CPAMD8, CPB1, CPEB1, CPEB1-AS1, CPEB2-AS1, CPLX2, CPLX3, CPNE4, CPNE5, CPNE6, CPNE9, CPS1-IT1, CPSF4L, CPT1C, CPXCR1, CPXM1, CPXM2, CPZ, CR1, CR1L, CR2, CRABP1, CRACR2A, CRAT37, CRAT40, CRATE, CRB1, CRB2, CREB3L1, CREG2, CRH, CRHBP, CRHR1, CRHR2, CRISP1, CRISP2, CRISP3, CRISPLD1, CRISPLD2, CRLF1, CRLF2, CRNN, CROCCP3, CRTAC1, CRTAM, CRTC3-AS1, CRX, CRYBA1, CRYBA2, CRYBA4, CRYBB1, CRYGA, CRYGB, CRYGC, CRYGD, CRYGN, CRYM-AS1, CSAG1, CSAG3, CSAG4, CSF1R, CSF2RA, CSF2RB, CSF3R, CSGALNACT1, CSH1, CSH2, CSHL1, CSMD1, CSMD2, CSMD2-AS1, CSMD3, CSN1S1, CSN1S2AP, CSN1S2BP, CSN2, CSN3, CSNK1G2-AS1, CSPG4, CSPG4P1Y, CSRNP3, CSRP3, CST1, CST11, CST13P, CST2, CST4, CST5, CST7, CST8, CST9, CST9L, CSTF3-AS1, CSTL1, CT45A1, CT45A10, CT45A4, CT45A6, CT45A7, CT47A1, CT47A10, CT47A11, CT47A6, CT47A7, CT47B1, CT55, CT62, CT83, CTAG1A, CTAG1B, CTAGE1, CTAGE10P, CTAGE11P, CTAGE6, CTAGE7P, CTB-113P19.1, CTB-1202.1, CTB-178M22.2, CTB-7E3.1, CTC-436P18.1, CTCFL, CTD-2151A2.1, CTD-2194D22.4, CTD-2201E9.1, CTD-2201I18.1, CTD-2270F17.1, CTD-2297D10.2, CTD-2350J17.1, CTD-3080P12.3, CTF1, CTGLF12P, CTHRC1, CTLA4, CTNNA2, CTNNA3, CTNND2, CTRB1, CTRB2, CTRC, CTSE, CTSG, CTSL3P, CTSLP2, CTSLP8, CTSV, CTSW, CTTNBP2, CTXN2, CTXN3, CUBN, CUZD1, CWH43, CX3CR1, CXADRP2, CXADRP3, CXCL14, CXCL17, CXCR1, CXCR2, CXCR2P1, CXCR3, CXCR5, CXCR6, CXXC1P1, CXXC4, CXorf21, CXorf36, CXorf49B, CXorf51A, CXorf58, CXorf65, CXorf66, CXorf67, CYBB, CYLC1, CYLC2, CYMP, CYP11B1, CYP11B2, CYP19A1, CYP1B1-AS1, CYP26C1, CYP27C1, CYP2A13, CYP2F1, CYP2G1P, CYP2S1, CYP2W1, CYP46A1, CYP4B1, CYP4F24P, CYP4F29P, CYP4F30P, CYP4F35P, CYP4F62P, CYP4F8, CYP4X1, CYP4Z1, CYP4Z2P, CYP51A1-AS1, CYS1, CYSLTR1, CYSLTR2, CYTH4, CYTIP, CYTL1, CYYR1, D21S2088E, DAAM2, DAB1, DAB1-AS1, DACH1, DACH2, DACT1, DACT2, DACT3-AS1, DAND5, DADA, DADA-AS1, DAPL1, DAPP1, DAW1, DAZ1, DAZ2, DAZ3, DAZ4, DAZL, DBET, DBH, DBIL5P, DBIL5P2, DBX1, DBX2, DCAF12L1, DCAF12L2, DCAF13P3, DCAF4L2, DCAF8L1, DCAF8L2, DCANP1, DCC, DCD, DCDC1, DCDC2B, DCDC2C, DCDC5, DCHS1, DCHS2, DCLK1, DCLK2, DCLK3, DCN, DCST1, DCSTAMP, DCT, DCTN1-AS1, DCX, DDC-AS1, DDI1, DDR2, DDX11-AS1, DDX11L1, DDX11L10, DDX11L16, DDX11L2, DDX11L5, DDX11L9, DDX12P, DDX25, DDX26B, DDX26B-AS1, DDX3Y, DDX4, DDX43, DDX53, DEF6, DEFA1, DEFA10P, DEFA11P, DEFA1B, DEFA3, DEFA4, DEFA5, DEFA6, DEFA8P, DEFA9P, DEFB103A, DEFB104A, DEFB104B, DEFB105A, DEFB107A, DEFB108B, DEFB109P1B, DEFB110, DEFB112, DEFB113, DEFB114, DEFB115, DEFB116, DEFB118, DEFB119, DEFB121, DEFB122, DEFB123, DEFB124, DEFB125, DEFB126, DEFB127, DEFB128, DEFB129, DEFB130, DEFB131, DEFB132, DEFB133, DEFB134, DEFB136, DEFB4A, DEFB4B, DEFT1P2, DEGS2, DENND2A, DENND3, DENND5B-AS1, DEPDC1, DEPDC1-AS1, DEPDC1B, DES, DGAT2L6, DGCR10, DGCR9, DGKB, DGKI, DGKK, DHFR, DHH, DHRS7C, DIAPH2-AS1, DIAPH3-AS1, DIAPH3-AS2, DIO2, DIO2-AS1, DIO3, DIO3OS, DIRAS1, DIRAS2, DIRC1, DIRC3, DISCI, DISC1-IT1, DISC1FP1, DISC2, DKFZP434A062, DKFZP434H168, DKFZP434K028, DKFZP434L187, DKFZp434J0226, DKFZp434L192, DKFZp451B082, DKFZp686K1684, DKK1, DKK2, DKK4, DKKL1, DLEC1, DLEU1-AS1, DLEU2L, DLEU7, DLEU7-AS1, DLG1-AS1, DLG2, DLG3-AS1, DLG5-AS1, DLGAP1, DLGAP1-AS3, DLGAP1-AS4, DLGAP1-ASS, DLGAP2, DLGAP2-AS1, DLGAP3, DLGAP4-AS1, DLGAP5, DLK1, DLK2, DLL1, DLL3, DLL4, DLX1, DLX2, DLX2-AS1, DLX3, DLX4, DLX5, DLL6, DLX6-AS1, DMBT1, DMBT1P1, DMBX1, DMC1, DMP1, DMRT1, DMRT2, DMRT3, DMRTA2, DMRTB1, DMRTC1B, DMRTC2, DNAH10, DNAH12, DNAH17, DNAH17-AS1, DNAH2, DNAH3, DNAH6, DNAH7, DNAH8, DNAH9, DNAI1, DNAI2, DNAJA1P5, DNAJB13, DNAJB3, DNAJB7, DNAJB8, DNAJB8-AS1, DNAJC25-GNG10, DNAJC27-AS1, DNAJC5B, DNAJC5G, DNAJC9-AS1, DNASE1L2, DNASE2B, DNER, DNM1, DNM1P35, DNM1P41, DNM1P46, DNM3, DNM3-IT1, DNM30S, DNTT, DOC2A, DOC2B, DOCK11, DOCK2, DOCK3, DOCK4-AS1, DOCKS, DOCK9-AS1, DOK2, DOK5, DOK6, DPCR1, DPEP1, DPEP2, DPEP3, DPF1, DPH3P1, DPP10, DPP10-AS1, DPP10-AS3, DPP6, DPPA2, DPPA2P3, DPPA3, DPPA4, DPPA5, DPRX, DPRXP4, DPT, DPY19L2, DPY19L2P1, DPY19L2P2, DPY19L2P3, DPY19L2P4, DPYD-AS1, DPYD-AS2, DPYSL3, DPYSL5, DQX1, DRAIN, DRC7, DRD1, DRD2, DRD5, DRGX, DRICH1, DRP2, DSC1, DSCAM, DSCAM-AS1, DSCAM-IT1, DSCAML1, DSCAS, DSCR10, DSCR4, DSCR8, DSCR9, DSG1-AS1, DSG2-AS1, DSG3, DSG4, DSPP, DTHD1, DUOX1, DUOXA1, DUPD1, DUSP15, DUSP19, DUSP21, DUSP26, DUSP27, DUSP4, DUSP5P1, DUX4, DUXA, DUXAP10, DUXAP8, DYDC1, DYNAP, DYNC2H1, DYNLRB2, DYTN, DYX1C1, DYX1C1-CCPG1, DZIP1, DZIP1L, E2F2, E2F8, EBF1, EBF2, EBF3, EBLN1, ECEL1, ECEL1P2, ECRP, ECSCR, ECT2L, EDA, EDAR, EDDM3A, EDDM3B, EDIL3, EDN3, EDNRA, EDNRB, EDNRB-AS1, EDRF1-AS1, EEF1DP3, EFCAB1, EFCAB10, EFCAB14-AS1, EFCAB3, EFCAB5, EFCAB6, EFCAB6-AS1, EFCAB9, EFCC1, EFEMP1, EFHB, EFHC2, EFNA5, EFNB3, EFR3B, EFS, EFTUD1P1, EGF, EGFEM1P, EGFL6, EGFLAM, EGFLAM-AS2, EGFLAM-AS4, EGR3, EGR4, EHD3, EHD4-AS1, EHF, EHHADH-AS1, EHMT1-IT1, EIF1AY, EIF1B-AS1, EIF2B5-AS1, EIF3C, EIF3CL, EIF4E1B, ELANE, ELAVL2, ELAVL3, ELAVL4, ELDR, ELF5, ELFN1-AS1, ELFN2, ELK2AP, ELMO1-AS1, ELMOD1, ELN, ELOVL4, ELSPBP1, EMB, EMBP1, EMCN, EME1, EMID1, EMILIN1, EMILIN2, EMILIN3, EMX1, EMX2, EMX2OS, EN1, EN2, ENDOU, ENOX1, ENOX1-AS2, ENPP3, ENPP5, ENPP6, ENPP7P13, ENTHD1, ENTPD1-AS1, ENTPD3, EOMES, EPB41L3, EPB41L4A-AS2, EPB42, EPGN, EPHA1-AS1, EPHA10, EPHA3, EPHA5, EPHA5-AS1, EPHA6, EPHA7, EPHA8, EPHB1, EPHB3, EPHX4, EPN2-AS1, EPN2-IT1, EPN3, EPPIN, EPPIN-WFDC6, EPS8L1, EPS8L3, EPX, EPYC, EQTN, ERAS, ERBB4, ERC2, ERC2-IT1, ERCC6L, EREG, ERG, ERICH1-AS1, ERICH3-AS1, ERICH4, ERICH6, ERICH6B, ERMN, ERN2, ERP27, ERVFRD-1, ERVH48-1, ERVMER34-1, ERW-1, ERW-2, ESCO2, ESM1, ESPL1, ESPNL, ESPNP, ESR1, ESR2, ESRG, ESRP1, ESRRB, ESRRG, ESX1, ESYT3, ETV3L, EVA1C, EVADR, EVC2, EVI2A, EVI2B, EVPL, EVX1, EVX1-AS, EVX2, EWSAT1, EXD1, EXO1, EXOC3L1, EXOC3L2, EXTL1, EXTL3-AS1, EYA1, EYA2, EYA4, EYS, EZR-AS1, F10-AS1, F11-AS1, F13A1, F2RL3, F8A2, FA2H, FAAHP1, FABP2, FABP5, FABP5P3, FABP6, FABP7, FABP9, FAIM2, FALEC, FAM101B, FAM105A, FAM106A, FAM106B, FAM106CP, FAM107A, FAM109B, FAM110D, FAM124A, FAM124B, FAM129C, FAM131C, FAM132B, FAM133A, FAM133CP, FAM133DP, FAM135B, FAM138B, FAM138C, FAM138D, FAM138E, FAM138F, FAM150A, FAM153A, FAM153B, FAM153C, FAM155A, FAM155A-IT1, FAM156B, FAM157A, FAM157B, FAM157C, FAM159A, FAM159B, FAM162B, FAM163A, FAM163B, FAM166A, FAM167A, FAM167A-AS1, FAM167B, FAM170A, FAM170B, FAM170B-AS1, FAM171A2, FAM172BP, FAM177B, FAM178B, FAM180A, FAM180B, FAM181A, FAM181A-AS1, FAM181B, FAM182A, FAM182B, FAM183B, FAM183CP, FAM184B, FAM186A, FAM186B, FAM187B, FAM189A1, FAM196B, FAM197Y2, FAM197Y5, FAM198B, FAM19A1, FAM19A2, FAM19A3, FAM19A4, FAM19A5, FAM205A, FAM205BP, FAM205C, FAM212B-AS1, FAM215A, FAM216B, FAM217A, FAM218A, FAM21EP, FAM221B, FAM224A, FAM224B, FAM225A, FAM225B, FAM226A, FAM227A, FAM228A, FAM230B, FAM230C, FAM231B, FAM231C, FAM24A, FAM24B-CUZD1, FAM25BP, FAM25G, FAM26D, FAM26E, FAM27B, FAM27C, FAM27E2, FAM27E3, FAM27L, FAM35BP, FAM35DP, FAM3B, FAM3D, FAM41AY1, FAM41C, FAM43A, FAM46B, FAM46D, FAM47A, FAM47B, FAM47C, FAM49A, FAM53A, FAM53B-AS1, FAM57B, FAM64A, FAM65B, FAM65C, FAM66A, FAM66B, FAM66C, FAM66D, FAM66E, FAM69C, FAM71B, FAM71C, FAM71D, FAM71E2, FAM71F1, FAM71F2, FAM72A, FAM72B, FAM72C, FAM72D, FAM74A3, FAM74A4, FAM74A6, FAM74A7, FAM78A, FAM81A, FAM81B, FAM83A, FAM83B, FAM83E, FAM83H-AS1, FAM86B1, FAM86B2, FAM87A, FAM87B, FAM90A1, FAM90A10P, FAM90A25P, FAM90A27P, FAM90A2P, FAM90A7P, FAM92A1P2, FAM92B, FAM95A, FAM95B1, FAM99B, FAM9A, FAM9B, FAM9C, FANCB, FANCD2, FANCD2OS, FANK1, FANK1-AS1, FAP, FAR1, FAR2, FAR2P1, FAR2P2, FAS-AS1, FAT2, FAT3, FATE1, FAXC, FBLN1, FBLN2, FBLN5, FBN2, FBN3, FBP2, FBXL13, FBXL16, FBXL2, FBXL22, FBXO15, FBXO24, FBXO3-AS1, FBXO39, FBXO40, FBXO41, FBXO43, FBXO47, FBXW10, FBXW12, FCAR, FCER1A, FCER2, FCGBP, FCGR1A, FCGR1B, FCGR1C, FCGR2A, FCGR2B, FCGR2C, FCGR3A, FCGR3B, FCHO1, FCMR, FCN1, FCN2, FCN3, FCRL1, FCRL2, FCRL3, FCRL4, FCRL5, FCRLB, FDCSP, FDPSP2, FENDRR, FER1L5, FER1L6, FER1L6-AS1, FER1L6-AS2, FERD3L, FEV, FEZF1, FEZF1-AS1, FEZF2, FFAR1, FFAR2, FFAR3, FFAR4, FGD2, FGD3, FGD5P1, FGF1, FGF10, FGF10-AS1, FGF12, FGF12-AS1, FGF13-AS1, FGF14-AS1, FGF14-IT1, FGF16, FGF17, FGF18, FGF20, FGF22, FGF23, FGF3, FGF4, FGF5, FGF6, FGF7, FGF8, FGF9, FGFBP1, FGFBP2, FGL2, FHAD1, FHL5, FHOD3, FIBCD1, FIGF, FIGLA, FILIP1, FIRRE, FKBP10, FKBP1A-SDCBP2, FKBP6, FKBP9P1, FKSG29, FLG, FLG2, FLJ12825, FLJ13224, FLJ16171, FLJ16779, FLJ22447, FLJ23867, FLJ25758, FLJ26245, FLJ26850, FLJ27354, FLJ30679, FLJ31104, FLJ31356, FLJ31662, FLJ32255, FLJ33360, FLJ33534, FLJ33581, FLJ34503, FLJ35934, FLJ36000, FLJ36777, FLJ37035, FLJ37201, FLJ37505, FLJ38122, FLJ38576, FLJ40194, FLJ40288, FLJ41200, FLJ41278, FLJ41941, FLJ42102, FLJ42393, FLJ42627, FLJ42969, FLJ43315, FLJ43879, FLJ45079, FLJ45513, FLJ46066, FLJ46284, FLRT1, FLT1, FLT3, FLT4, FMN1, FMN2, FMNL1, FMNL3, FMO2, FMO6P, FMO9P, FMOD, FMR1-AS1, FMR1NB, FNDC1, FNDC7, FNDC8, FNDC9, FOLH1B, FOLR1, FOLR2, FOXB1, FOXB2, FOXC2, FOXC2-AS1, FOXCUT, FOXD1, FOXD2, FOXD3, FOXD3-AS1, FOXD4, FOXD4L1, FOXD4L3, FOXD4L4, FOXD4L5, FOXD4L6, FOXE1, FOXF1, FOXF2, FOXG1, FOXG1-AS1, FOXH1, FOXI1, FOXI2, FOXI3, FOXJ1, FOXL1, FOXL2, FOXL2NB, FOXM1, FOXN1, FOXN3-AS2, FOXN4, FOXO3B, FOXO6, FOXP1-AS1, FOXP3, FOXR1, FOXR2, FOXS1, FPGT-TNNI3K, FPR1, FPR2, FPR3, FRAS1, FREM1, FREM2, FREM3, FRG2, FRG2B, FRG2C, FRG2DP, FRG2EP, FRMD1, FRMD5, FRMD6-AS2, FRMD7, FRMD8P1, FRMPD1, FRMPD2, FRMPD2B, FRMPD3, FRMPD3-AS1, FRMPD4, FRRS1L, FRY-AS1, FSCB, FSCN2, FSCN3, FSD1, FSHB, FSHR, FSIP2, FSTL5, FTH1P18, FTHL17, FTLP10, FTMT, FTO-IT1, FUNDC2P2, FUT3, FUT5, FUT7, FUT8-AS1, FUT9, FXYD3, FXYD4, FXYD6, FXYD6-FXYD2, FXYD7, FYN, FZD10, FZD10-AS1, FZD2, FZD3, FZD9, G6PC2, GAB3, GAB4, GABARAPL3, GABBR1, GABBR2, GABRA1, GABRA2, GABRA3, GABRA4, GABRA5, GABRA6, GABRB1, GABRB2, GABRB3, GABRD, GABRG1, GABRG2, GABRG3, GABRG3-AS1, GABRP, GABRQ, GABRR1, GABRR3, GACAT1, GACAT2, GACAT3, GAD2, GADL1, GAGE1, GAGE10, GAGE12D, GAGE12E, GAGE12F, GAGE12H, GAGE12I, GAGE12J, GAGE2C, GAGE2D, GAGE2E, GAGE4, GAGES, GAGE6, GAGE7, GAGER, GAL3ST2, GAL3ST3, GAL3ST4, GALNT12, GALNT13, GALNT14, GALNT16, GALNT3, GALNT5, GALNT6, GALNT8, GALNT9, GALNTL5, GALNTL6, GALP, GALR1, GALR3, GAP43, GAPLINC, GAPT, GAREML, GAS1, GAS2L1P2, GAS2L2, GAS6-AS2, GAS7, GAS8-AS1, GATA1, GATA2, GATA2-AS1, GATA3-AS1, GATA5, GBAT2, GBGT1, GBP1P1, GBP6, GBX1, GBX2, GCG, GCK, GCM2, GCNT1, GCNT7, GCOM1, GCSAML, GCSAML-AS1, GDAP1L1, GDF10, GDF11, GDF2, GDF3, GDF5, GDF6, GDPD2, GDPD4, GFAP, GFI1, GFI1B, GFRA2, GFRA3, GFRA4, GFRAL, GFY, GGN, GGNBP1, GGT3P, GGT5, GGT6, GGT8P, GGTA1P, GGTLC1, GGTLC2, GH1, GH2, GHET1, GHRH, GHRHR, GHSR, GIF, GIMAP1, GIMAP1-GIMAP5, GIMAP4, GIMAP5, GIMAP6, GIMAP7, GIMAP8, GIMD1, GIP, GIPC3, GIPR, GJA1, GJA10, GJA3, GJA4, GJA5, GJA8, GJA9, GJB4, GJB5, GJB6, GJB7, GJC2, GJD2, GJD4, GK2, GK3P, GKN1, GKN2, GLB1L2, GLB1L3, GLDN, GLI2, GLI3, GLIPR1L1, GLIPR1L2, GLIS1, GLIS3-AS1, GLP1R, GLP2R, GLRA1, GLRA2, GLRA3, GLRA4, GLT6D1, GLYATL2, GLYATL3, GLYCAM1, GM140, GMCL1P1, GML, GMNC, GNA14-AS1, GNA15, GNAS-AS1, GNAT1, GNAT2, GNAT3, GNB3, GNB4, GNG11, GNG13, GNG2, GNG3, GNG4, GNGT1, GNGT2, GNRH2, GNRHR, GNRHR2, GOLGA2P2Y, GOLGA2P6, GOLGA2P7, GOLGA2P9, GOLGA6A, GOLGA6B, GOLGA6C, GOLGA6D, GOLGA6L1, GOLGA6L10, GOLGA6L17P, GOLGA6L2, GOLGA6L22, GOLGA6L3, GOLGA6L4, GOLGA6L5P, GOLGA6L6, GOLGA6L7P, GOLGA6L9, GOLGA8DP, GOLGA8EP, GOLGA8F, GOLGA8G, GOLGA8H, GOLGA8J, GOLGA8K, GOLGA8M, GOLGA8N, GOLGA80, GOLGA8R, GOLGA8S, GOLGA8T, GOT1L1, GP1BA, GP1BB, GP2, GP6, GP9, GPA33, GPAT2, GPC2, GPC4, GPC5, GPC5-AS1, GPC5-AS2, GPC6-AS2, GPHA2, GPHB5, GPIHBP1, GPM6A, GPM6B, GPR1, GPR1-AS, GPR101, GPR119, GPR12, GPR132, GPR137C, GPR139, GPR141, GPR142, GPR143, GPR148, GPR149, GPR15, GPR150, GPR151, GPR152, GPR156, GPR158, GPR158-AS1, GPR162, GPR171, GPR173, GPR174, GPR179, GPR18, GPR182, GPR19, GPR20, GPR21, GPR22, GPR25, GPR26, GPR27, GPR3, GPR31, GPR32, GPR33, GPR34, GPR4, GPR45, GPR50, GPR52, GPR55, GPR6, GPR61, GPR62, GPR63, GPR65, GPR68, GPR78, GPR82, GPR83, GPR84, GPR85, GPR88, GPR89B, GPRC5D, GPRC6A, GPRIN2, GPRIN3, GPSM1, GPSM2, GPSM3, GPX5, GPX6, GPX8, GRAMD1B, GRAMD2, GRAP, GRAP2, GRAPL, GRASP, GREB1, GRHL2, GRHL3, GRIA1, GRIA2, GRIA4, GRID1-AS1, GRID2, GRID2IP, GRIFIN, GRIK1, GRIK1-AS1, GRIK1-AS2, GRIK2, GRIK3, GRIK4, GRIK5, GRIN1, GRIN2A, GRIN2B, GRIN2C, GRIN2D, GRIN3A, GRIN3B, GRIP1, GRIP2, GRK1, GRK7, GRM1, GRM2, GRM3, GRM4, GRM5, GRM5-AS1, GRM6, GRM7, GRM7-AS1, GRM7-AS2, GRM7-AS3, GRP, GRXCR1, GRXCR2, GS1-24F4.2, GS1-279B7.1, GS1-594A7.3, GS1-600G8.3, GSC, GSC2, GSDMA, GSDMC, GSG1, GSG1L, GSC2, GSN-AS1, GSTA3, GSTM2P1, GSTM5, GSTT1, GSTT2B, GSTTP1, GSTTP2, GSX1, GTF2A1L, GTF2H2B, GTF2H2C, GTF2IP1, GTF2IP4, GTF2IRD1P1, GTF2IRD2, GTF3C2-AS1, GTSCR1, GTSE1-AS1, GTSF1, GTSF1L, GUCA1A, GUCA1C, GUCY1A2, GUCY1B2, GUCY2C, GUCY2D, GUCY2EP, GUCY2F, GUCY2GP, GULP1, GUSBP10, GUSBP2, GUSBP3, GUSBP5, GUSBP9, GVINP1, GXYLT1P3, GYG2P1, GYLTL1B, GYPA, GYPB, GYPE, GZMA, GZMB, GZMH, GZMK, H1FNT, H1F00, H1FX-AS1, H2AFB2, H2AFB3, H2BFM, H2BFWT, H2BFXP, H3F3A, HACD4, HAGLR, HAGLROS, HAND1, HAND2, HAND2-AS1, HAO2-IT1, HAP1, HAPLN1, HAPLN2, HAPLN4, HAR1A, HAR1B, HAS1, HAS2, HAS2-AS1, HAVCR1P1, HBBP1, HBD, HBE1, HBG1, HBG2, HBM, HBQ1, HBZ, HCAR1, HCAR2, HCCAT5, HCFC1-AS1, HCG11, HCG14, HCG2040054, HCG22, HCG23, HCK, HCLS1, HCN1, HCN2, HCRT, HCRTR1, HCRTR2, HDAC11-AS1, HDAC9, HDC, HDGFL1, HDGFRP3, HEATR4, HEATR9, HECTD2-AS1, HECW1, HECW2, HELT, HEMGN, HENMT1, HEPACAM, HEPACAM2, HEPH, HEPHL1, HEPN1, HERC2P4, HERC2P7, HES3, HEST, HEXA-AS1, HEYL, HFM1, HGC6.3, HGF, HHIP, HHIP-AS1, HHIPL1, HHIPL2, HHLA1, HHLA2, HID1-AS1, HIF1A-AS1, HIF3A, HIGD1B, HIGD1C, HIGD2B, HILS1, HIPK4, HIST1H1B, HIST1H1D, HIST1H2AA, HIST1H2AJ, HIST1H2APS1, HIST1H2BA, HIST1H2BB, HIST1H2BH, HIST1H2BI, HIST1H2BL, HIST1H2BM, HIST1H3C, HIST1H3F, HIST1H3G, HIST1H3I, HIST1H4A, HIST1H4F, HIST1H4G, HIST1H4I, HIST1H4J, HIST1H4K, HIST1H4L, HIST2H2AA4, HIST2H2AB, HIST2H2BA, HIST2H3A, HIST2H3D, HIST2H4A, HIST3H2BB, HIST3H3, HJURP, HK2, HK3, HLA-DOA, HLA-DQA1, HLA-DQA2, HLA-DQB1, HLA-DQB2, HLA-DRB3, HLA-DRB4, HLA-DRB5, HLA-DRB6, HLTF-AS1, HLX-AS1, HMCN1, HMCN2, HMGA1P7, HMGA2, HMGB3P1, HMGB4, HMGCLL1, HMHB1, HMMR-AS1, HMSD, HMX1, HMX2, HMX3, HNCAT21, HNRNPA1P10, HNRNPA3P1, HNRNPCL1, HNRNPCL2, HNRNPCL4, HNRNPKP3, HOPX, HORMAD1, HORMAD2, HOTAIR, HOTAIRM1, HOTS, HOTTIP, HOXA-AS2, HOXA-AS3, HOXA1, HOXA10, HOXA10-AS, HOXA10-HOXA9, HOXA11, HOXA11-AS, HOXA13, HOXA2, HOXA3, HOXA4, HOXA5, HOXA6, HOXA7, HOXA9, HOXB-AS1, HOXB-AS3, HOXB1, HOXB13, HOXB2, HOXB3, HOXB4, HOXB5, HOXB6, HOXB7, HOXB8, HOXB9, HOXC-AS1, HOXC-AS2, HOXC-AS3, HOXC10, HOXC11, HOXC12, HOXC13, HOXC13-AS, HOXC4, HOXC5, HOXC6, HOXC8, HOXC9, HOXD-AS2, HOXD1, HOXD10, HOXD11, HOXD12, HOXD13, HOXD3, HOXD4, HOXD8, HOXD9, HP09025, HP09053, HPCA, HPCAL4, HPGDS, HPSE, HPSE2, HPVC1, HPYR1, HR, HRASLS, HRASLS5, HRAT13, HRAT17, HRAT5, HRAT56, HRAT92, HRC, HRH1, HRH2, HRH3, HRH4, HRK, HRNR, HS3ST1, HS3ST3A1, HS3ST4, HS3ST5, HS6ST2, HS6ST2-AS1, HS6ST3, HSD3B1, HSD3B2, HSD3BP4, HSD52, HSF2BP, HSF5, HSFX2, HSFY1P1, HSFY2, HSH2D, HSP90AB4P, HSPA12B, HSPB2, HSPB2-C11orf52, HSPB3, HSPB6, HSPB7, HSPE1-MOB4, HTN1, HTN3, HTR1A, HTR1B, HTR1D, HTR1E, HTRIF, HTR2A, HTR2A-AS1, HTR2B, HTR2C, HTR3A, HTR3B, HTR3C, HTR3D, HTR3E, HTR4, HTR5A, HTR5A-AS1, HTR6, HTR7, HTRA3, HTRA4, HTT-AS, HUS1B, HVCN1, HYAL4, HYALP1, HYDIN, HYDIN2, HYMAI, HYPM, IBA57-AS1, IBSP, ICA1L, ICOS, ID4, IFIT1B, IFITM5, IFNA1, IFNA10, IFNA13, IFNA14, IFNA16, IFNA17, IFNA2, IFNA21, IFNA22P, IFNA4, IFNA5, IFNA6, IFNA7, IFNA8, IFNB1, IFNE, IFNG, IFNG-AS1, IFNK, IFNL1, IFNL2, IFNL3, IFNL4, IFNW1, IGDCC3, IGF1R, IGF2-AS, IGF2BP1, IGF2BP2, IGF2BP2-AS1, IGFBP5, IGFBP7, IGFBP7-AS1, IGFL2, IGFL3, IGFL4, IGFN1, IGLL1, IGLL3P, IGLL5, IGLON5, IGSF1, IGSF10, IGSF11, IGSF11-AS1, IGSF21, IGSF22, IGSF5, IGSF6, IGSF9B, IKZF1, IKZF2, IL10, IL10RA, IL12A-AS1, IL12B, IL12RB1, IL12RB2, IL13, IL13RA2, IL16, IL17A, IL17B, IL17D, IL17F, IL17RD, IL17REL, IL18RAP, IL19, IL1A, IL1F10, IL1RAPL1, IL1RAPL2, IL2, IL20, IL20RA, IL21, IL21-AS1, IL21R, IL21R-AS1, IL22, IL22RA2, IL23R, IL24, IL25, IL26, IL2RA, IL2RB, IL2RG, IL3, IL31, IL31RA, IL33, IL34, IL36A, IL36G, IL36RN, IL37, IL4, IL4I1, IL5, IL5RA, IL9, IL9R, ILDR1, IMPG1, IMPG2, INA, INE2, INGX, INHA, INHBA-AS1, INMT-FAM188B, INPP4B, INPP5J, INS, INSL3, INSL4, INSL6, INSM1, INSM2, INSRR, INTS4P2, IPCEF1, IPO9-AS1, IQCA1, IQCA1L, IQCF1, IQCF2, IQCF3, IQCF4, IQCF5, IQCF5-AS1, IQCF6, IQCH, IQCJ, IQCJ-SCHIP1-AS1, IQGAP3, IQSEC2, IQSEC3, IQUB, IRAIN, IRAK1BP1, IRAK3, IRG1, IRGC, IRGM, IRS4, IRX1, IRX2, IRX4, IRX5, IRX6, ISL1, ISL2, ISLR, ISLR2, ISM2, ISPD-AS1, ISX, ITFG1-AS1, ITGA10, ITGA11, ITGA2B, ITGA4, ITGA8, ITGAD, ITGAX, ITGB2-AS1, ITGB4, ITGB8, ITGBL1, ITIH5, ITIH6, ITK, ITLN1, ITM2A, ITPK1-AS1, ITPKB, ITPKB-IT1, ITPR1-AS1, ITPRIPL1, IZUMO1, IZUMO1R, IZUMO2, IZUMO3, JAK3, JAKMIP1, JAKMIP2, JAKMIP2-AS1, JAKMIP3, JAM2, JAM3, JARID2-AS1, JAZF1-AS1, JCHAIN, JMJD1C-AS1, JPH2, JPH3, JPH4, JRKL-AS1, KAAG1, KANTR, KATNAL2, KATNBL1P6, KAZALD1, KAZN, KBTBD12, KBTBD13, KC6, KCNA1, KCNA10, KCNA2, KCNA3, KCNA4, KCNA5, KCNA6, KCNA7, KCNAB1, KCNAB1-AS1, KCNAB1-AS2, KCNAB2, KCNB1, KCNB2, KCNC1, KCNC2, KCNC4-AS1, KCND3-AS1, KCND3-IT1, KCNE1, KCNE3, KCNE4, KCNF1, KCNG1, KCNC2, KCNG3, KCNG4, KCNH1, KCNH2, KCNH3, KCNH4, KCNH5, KCNH6, KCNH7, KCNH8, KCNIP1, KCNIP4, KCNIP4-IT1, KCNJ1, KCNJ10, KCNJ12, KCNJ13, KCNJ15, KCNJ16, KCNJ18, KCNJ2, KCNJ2-AS1, KCNJ3, KCNJ4, KCNJ5, KCNJ6, KCNJ9, KCNK10, KCNK12, KCNK13, KCNK15, KCNK16, KCNK17, KCNK18, KCNK2, KCNK3, KCNK4, KCNK6, KCNK7, KCNK9, KCNMA1-AS1, KCNMA1-AS2, KCNMA1-AS3, KCNMB1, KCNMB2, KCNMB4, KCNN1, KCNN3, KCNN4, KCNQ1, KCNQ1-AS1, KCNQ1DN, KCNQ1OT1, KCNQ2, KCNQ3, KCNQ4, KCNQ5, KCNQ5-AS1, KCNQ5-IT1, KCNRG, KCNS1, KCNS2, KCNT1, KCNU1, KCNV1, KCNV2, KCTD12, KCTD14, KCTD16, KCTD19, KCTD4, KCTD8, KDM4D, KDM4E, KDM5D, KDR, KEL, KERA, KGFLP1, KGFLP2, KHDC1, KHDC1L, KHDC3L, KHDRBS2, KIAA0087, KIAA0101, KIAA0125, KIAA0226L, KIAA0319, KIAA0408, KIAA0825, KIAA1024, KIAA1024L, KIAA1210, KIAA1211, KIAA1211L, KIAA1257, KIAA1324L, KIAA1328, KIAA1456, KIAA1549L, KIAA1614, KIAA1644, KIAA1656, KIAA1755, KIAA1875, KIAA2012, KIAA2022, KIF11, KIF14, KIF15, KIF17, KIF18A, KIF18B, KIF19, KIF1A, KIF20A, KIF24, KIF25, KIF25-AS1, KIF26A, KIF26B, KIF27, KIF2B, KIF2C, KIF4A, KIF4B, KIF5A, KIF5C, KIF6, KIF7, KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, KIR3DS1, KIR3DX1, KIRREL2, KIRREL3, KIRREL3-AS2, KIRREL3-AS3, KISS1R, KIT, KIZ-AS1, KLF1, KLF14, KLF17, KLF3-AS1, KLF8, KLHDC1, KLHDC7B, KLHDC8A, KLHL1, KLHL10, KLHL14, KLHL30, KLHL32, KLHL33, KLHL34, KLHL35, KLHL38, KLHL40, KLHL41, KLHL6, KLHL6-AS1, KLHL7-AS1, KLK1, KLK10, KLK11, KLK12, KLK13, KLK14, KLK15, KLK2, KLK3, KLK4, KLK5, KLK6, KLK7, KLK8, KLK9, KLKP1, KLRB1, KLRC1, KLRC2, KLRC3, KLRC4, KLRD1, KLRF1, KLRF2, KLRC2, KLRK1, KNCN, KPRP, KRBOX1-AS1, KREMEN2, KRT1, KRT12, KRT13, KRT14, KRT15, KRT16, KRT16P1, KRT16P2, KRT16P3, KRT17P5, KRT18P55, KRT19P2, KRT2, KRT20, KRT23, KRT24, KRT25, KRT26, KRT27, KRT28, KRT3, KRT31, KRT32, KRT33A, KRT33B, KRT34, KRT35, KRT36, KRT37, KRT38, KRT39, KRT4, KRT40, KRT42P, KRT5, KRT6A, KRT6B, KRT6C, KRT71, KRT72, KRT73, KRT73-AS1, KRT74, KRT75, KRT76, KRT77, KRT78, KRT79, KRT82, KRT83, KRT84, KRT85, KRT86, KRT8P41, KRT9, KRTAP1-1, KRTAP1-3, KRTAP1-4, KRTAP1-5, KRTAP10-1, KRTAP10-10, KRTAP10-11, KRTAP10-12, KRTAP10-2, KRTAP10-3, KRTAP10-4, KRTAP10-5, KRTAP10-6, KRTAP10-7, KRTAP10-8, KRTAP10-9, KRTAP11-1, KRTAP12-1, KRTAP12-2, KRTAP12-3, KRTAP12-4, KRTAP13-1, KRTAP13-2, KRTAP13-3, KRTAP13-4, KRTAP15-1, KRTAP16-1, KRTAP17-1, KRTAP19-1, KRTAP19-2, KRTAP19-3, KRTAP19-4, KRTAP19-5, KRTAP19-6, KRTAP19-7, KRTAP19-8, KRTAP2-1, KRTAP2-2, KRTAP2-4, KRTAP20-1, KRTAP20-2, KRTAP20-3, KRTAP20-4, KRTAP21-1, KRTAP21-3, KRTAP22-1, KRTAP22-2, KRTAP23-1, KRTAP24-1, KRTAP25-1, KRTAP26-1, KRTAP27-1, KRTAP29-1, KRTAP3-1, KRTAP3-2, KRTAP3-3, KRTAP4-1, KRTAP4-11, KRTAP4-12, KRTAP4-2, KRTAP4-3, KRTAP4-4, KRTAP4-5, KRTAP4-6, KRTAP4-7, KRTAP4-8, KRTAP4-9, KRTAP5-1, KRTAP5-10, KRTAP5-11, KRTAP5-2, KRTAP5-3, KRTAP5-4, KRTAP5-5, KRTAP5-6, KRTAP5-7, KRTAP5-9, KRTAP6-1, KRTAP6-2, KRTAP6-3, KRTAP7-1, KRTAP8-1, KRTAP9-1, KRTAP9-2, KRTAP9-3, KRTAP9-4, KRTAP9-6, KRTAP9-8, KRTAP9-9, KRTDAP, KU-MEL-3, KY, L1CAM, L1TD1, L3MBTL4-AS1, LACAT8, LACRT, LACTB2-AS1, LACTBL1, LAIR1, LAIR2, LALBA, LAMA1, LAMA2, LAMB4, LAMC3, LAMPS, LAMP5-AS1, LANCL1-AS1, LANCL3, LARGE-AS1, LARS2-AS1, LAX1, LAYN, LBX1, LBX1-AS1, LCA10, LCA5, LCA5L, LCE1A, LCE1B, LCE1C, LCE1D, LCE1E, LCE1F, LCE2A, LCE2B, LCE2C, LCE2D, LCE3A, LCE3B, LCE3C, LCE3E, LCE4A, LCE5A, LCE6A, LCK, LCMT1-AS2, LCN1, LCN10, LCN6, LCN8, LCN9, LCNL1, LCP2, LCT, LCTL, LDB2, LDB3, LDHAL6A, LDHAL6B, LDHC, LDLRAD1, LDLRAD2, LDLRAD4-AS1, LDOC1, LECT1, LEF1, LEF1-AS1, LEFTY2, LEKR1, LELP1, LEMD1, LEMD1-AS1, LENEP, LEP, LEUTX, LGALS12, LGALS13, LGALS14, LGALS16, LGALS17A, LGALS2, LGALS7, LGALS7B, LGALS9B, LGI1, LGI2, LGI3, LGI4, LGR6, LGSN, LHB, LHCGR, LHFPL1, LHFPL3, LHFPL3-AS1, LHFPL4, LHFPL5, LHX1, LHX2, LHX3, LHX4, LHX5, LHX5-AS1, LHX6, LHX8, LHX9, LIFR-AS1, LILRA1, LILRA2, LILRA3, LILRA4, LILRA5, LILRA6, LILRB1, LILRB2, LILRB3, LILRB4, LILRB5, LILRP2, LIM2, LIMD2, LIMS3-LOC440895, LIN28A, LIN28B, LINC-ROR, LINC00028, LINC00029, LINC00032, LINC00051, LINC00052, LINC00092, LINC00102, LINC00111, LINC00112, LINC00113, LINC00114, LINC00158, LINC00160, LINC00161, LINC00162, LINC00163, LINC00167, LINC00173, LINC00176, LINC00184, LINC00200, LINC00202-1, LINC00202-2, LINC00207, LINC00208, LINC00210, LINC00211, LINC00221, LINC00222, LINC00226, LINC00229, LINC00238, LINC00239, LINC00243, LINC00244, LINC00251, LINC00254, LINC00264, LINC00265, LINC00266-1, LINC00266-3, LINC00269, LINC00271, LINC00272, LINC00273, LINC00276, LINC00278, LINC00280, LINC00282, LINC00284, LINC00290, LINC00293, LINC00297, LINC00298, LINC00299, LINC00301, LINC00303, LINC00304, LINC00305, LINC00307, LINC00308, LINC00309, LINC00310, LINC00314, LINC00316, LINC00317, LINC00319, LINC00320, LINC00322, LINC00323, LINC00326, LINC00327, LINC00330, LINC00331, LINC00332, LINC00333, LINC00336, LINC00337, LINC00343, LINC00346, LINC00347, LINC00348, LINC00350, LINC00351, LINC00353, LINC00354, LINC00358, LINC00359, LINC00363, LINC00364, LINC00366, LINC00367, LINC00368, LINC00371, LINC00375, LINC00376, LINC00377, LINC00378, LINC00379, LINC00380, LINC00381, LINC00382, LINC00383, LINC00392, LINC00395, LINC00396, LINC00397, LINC00398, LINC00399, LINC00400, LINC00403, LINC00408, LINC00410, LINC00411, LINC00412, LINC00417, LINC00421, LINC00423, LINC00424, LINC00426, LINC00428, LINC00431, LINC00433, LINC00434, LINC00437, LINC00440, LINC00441, LINC00442, LINC00443, LINC00445, LINC00446, LINC00448, LINC00452, LINC00456, LINC00457, LINC00458, LINC00460, LINC00461, LINC00462, LINC00463, LINC00466, LINC00469, LINC00470, LINC00471, LINC00472, LINC00473, LINC00474, LINC00475, LINC00477, LINC00479, LINC00483, LINC00485, LINC00486, LINC00487, LINC00488, LINC00489, LINC00491, LINC00492, LINC00494, LINC00499, LINC00501, LINC00502, LINC00504, LINC00506, LINC00507, LINC00508, LINC00511, LINC00514, LINC00515, LINC00518, LINC00520, LINC00521, LINC00523, LINC00524, LINC00534, LINC00535, LINC00536, LINC00538, LINC00539, LINC00540, LINC00544, LINC00545, LINC00547, LINC00548, LINC00550, LINC00551, LINC00552, LINC00554, LINC00557, LINC00558, LINC00559, LINC00562, LINC00563, LINC00564, LINC00565, LINC00566, LINC00570, LINC00572, LINC00575, LINC00577, LINC00578, LINC00581, LINC00582, LINC00583, LINC00587, LINC00588, LINC00592, LINC00593, LINC00598, LINC00599, LINC00601, LINC00602, LINC00603, LINC00606, LINC00608, LINC00609, LINC00612, LINC00613, LINC00615, LINC00616, LINC00618, LINC00619, LINC00620, LINC00622, LINC00624, LINC00626, LINC00629, LINC00632, LINC00633, LINC00634, LINC00635, LINC00636, LINC00637, LINC00638, LINC00639, LINC00640, LINC00642, LINC00643, LINC00644, LINC00645, LINC00648, LINC00652, LINC00654, LINC00656, LINC00658, LINC00661, LINC00664, LINC00665, LINC00668, LINC00669, LINC00670, LINC00671, LINC00673, LINC00675, LINC00676, LINC00678, LINC00681, LINC00682, LINC00683, LINC00687, LINC00689, LINC00690, LINC00691, LINC00692, LINC00693, LINC00696, LINC00698, LINC00700, LINC00701, LINC00703, LINC00705, LINC00706, LINC00707, LINC00708, LINC00709, LINC00710, LINC00824, LINC00836, LINC00837, LINC00838, LINC00839, LINC00840, LINC00841, LINC00845, LINC00850, LINC00851, LINC00853, LINC00854, LINC00858, LINC00861, LINC00864, LINC00865, LINC00866, LINC00867, LINC00870, LINC00871, LINC00877, LINC00879, LINC00882, LINC00885, LINC00887, LINC00889, LINC00890, LINC00892, LINC00893, LINC00894, LINC00895, LINC00896, LINC00898, LINC00900, LINC00901, LINC00904, LINC00905, LINC00906, LINC00907, LINC00908, LINC00911, LINC00917, LINC00919, LINC00921, LINC00922, LINC00923, LINC00924, LINC00925, LINC00927, LINC00928, LINC00929, LINC00930, LINC00933, LINC00934, LINC00935, LINC00936, LINC00937, LINC00939, LINC00940, LINC00941, LINC00942, LINC00943, LINC00944, LINC00945, LINC00950, LINC00951, LINC00954, LINC00955, LINC00957, LINC00958, LINC00959, LINC00961, LINC00964, LINC00965, LINC00967, LINC00968, LINC00970, LINC00971, LINC00974, LINC00977, LINC00982, LINC00989, LINC00992, LINC00993, LINC00994, LINC00996, LINC01001, LINC01002, LINC01005, LINC01007, LINC01010, LINC01012, LINC01013, LINC01014, LINC01015, LINC01016, LINC01017, LINC01019, LINC01020, LINC01024, LINC01028, LINC01029, LINC01030, LINC01031, LINC01032, LINC01033, LINC01036, LINC01038, LINC01039, LINC01040, LINC01044, LINC01046, LINC01047, LINC01048, LINC01049, LINC01050, LINC01053, LINC01054, LINC01055, LINC01056, LINC01058, LINC01060, LINC01065, LINC01068, LINC01069, LINC01070, LINC01072, LINC01075, LINC01080, LINC01081, LINC01082, LINC01085, LINC01087, LINC01088, LINC01090, LINC01091, LINC01095, LINC01096, LINC01097, LINC01098, LINC01099, LINC01100, LINC01101, LINC01102, LINC01103, LINC01104, LINC01105, LINC01106, LINC01107, LINC01108, LINC01111, LINC01114, LINC01115, LINC01116, LINC01117, LINC01118, LINC01119, LINC01120, LINC01121, LINC01122, LINC01123, LINC01124, LINC01132, LINC01133, LINC01134, LINC01135, LINC01136, LINC01139, LINC01141, LINC01142, LINC01143, LINC01144, LINC01150, LINC01152, LINC01158, LINC01159, LINC01160, LINC01162, LINC01163, LINC01166, LINC01167, LINC01168, LINC01169, LINC01170, LINC01177, LINC01179, LINC01180, LINC01182, LINC01185, LINC01187, LINC01189, LINC01191, LINC01192, LINC01193, LINC01194, LINC01195, LINC01197, LINC01198, LINC01201, LINC01202, LINC01203, LINC01204, LINC01205, LINC01206, LINC01207, LINC01208, LINC01209, LINC01210, LINC01212, LINC01213, LINC01215, LINC01216, LINC01219, LINC01220, LINC01221, LINC01222, LINC01224, LINC01225, LINC01226, LINC01227, LINC01230, LINC01231, LINC01232, LINC01233, LINC01234, LINC01241, LINC01242, LINC01246, LINC01247, LINC01248, LINC01249, LINC01250, LINC01251, LINC01252, LINC01254, LINC01255, LINC01256, LINC01257, LINC01258, LINC01260, LINC01262, LINC01264, LINC01265, LINC01266, LINC01267, LINC01271, LINC01272, LINC01276, LINC01277, LINC01279, LINC01280, LINC01281, LINC01282, LINC01284, LINC01285, LINC01287, LINC01288, LINC01289, LINC01291, LINC01296, LINC01298, LINC01299, LINC01300, LINC01301, LINC01304, LINC01305, LINC01307, LINC01309, LINC01310, LINC01312, LINC01317, LINC01322, LINC01324, LINC01327, LINC01330, LINC01331, LINC01333, LINC01335, LINC01337, LINC01338, LINC01339, LINC01340, LINC01341, LINC01342, LINC01343, LINC01344, LINC01346, LINC01347, LINC01349, LINC01350, LINC01351, LINC01352, LINC01353, LINC01354, LINC01356, LINC01359, LINC01360, LINC01361, LINC01363, LINC01364, LINC01365, LINC01366, LINC01375, LINC01377, LINC01378, LINC01384, LINC01386, LINC01387, LINC01389, LINC01391, LINC01392, LINC01393, LINC01395, LINC01396, LINC01397, LINC01398, LINC01399, LINC01402, LINC01405, LINC01410, LINC01411, LINC01412, LINC01413, LINC01416, LINC01419, LINC01422, LINC01423, LINC01424, LINC01425, LINC01427, LINC01429, LINC01432, LINC01436, LINC01440, LINC01441, LINC01443, LINC01444, LINC01445, LINC01446, LINC01448, LINC01449, LINC01450, LINC01455, LINC01461, LINC01467, LINC01468, LINC01470, LINC01471, LINC01473, LINC01475, LINC01476, LINC01477, LINC01478, LINC01479, LINC01480, LINC01482, LINC01483, LINC01484, LINC01486, LINC01487, LINC01488, LINC01489, LINC01490, LINC01491, LINC01492, LINC01493, LINC01494, LINC01495, LINC01496, LINC01497, LINC01498, LINC01499, LINC01500, LINC01501, LINC01502, LINC01504, LINC01505, LINC01506, LINC01507, LINC01508, LINC01509, LINC01511, LINC01514, LINC01516, LINC01517, LINC01518, LINC01519, LINC01520, LINC01521, LINC01522, LINC01523, LINC01524, LINC01525, LINC01526, LINC01529, LINC01530, LINC01531, LINC01532, LINC01533, LINC01535, LINC01537, LINC01538, LINC01539, LINC01541, LINC01543, LINC01544, LINC01546, LINC01548, LINC01549, LINC01550, LINC01551, LINC01552, LINC01553, LINC01555, LINC01556, LINC01561, LINC01563, LINC01565, LINC01566, LINC01567, LINC01568, LINC01570, LINC01571, LINC01572, LINC01574, LINC01580, LINC01581, LINC01582, LINC01583, LINC01584, LINC01585, LINC01586, LINC01590, LINC01591, LINC01592, LINC01594, LINC01600, LINC01602, LINC01603, LINC01605, LINC01606, LINC01607, LINC01608, LINC01609, LINC01611, LINCMD1, LINCR-0003, LINGO1, LINGO1-AS1, LINGO1-AS2, LINGO2, LINGO3, LINGO4, LIPE, LIPE-AS1, LIPF, LIPI, LIPJ, LIPK, LIPM, LIPN, LIX1, LKAAEAR1, LL22NC01-81G9.3, LL22NC03-63E9.3, LL22NC03-75H12.2, LMAN1L, LMCD1-AS1, LMF1-AS1, LMLN-AS1, LMNTD1, LMO1, LMO2, LMO3, LMO7-AS1, LMO7DN, LMO7DN-IT1, LMOD1, LMOD2, LMOD3, LMTK3, LMX1A, LMX1B, LNX1-AS1, LNX1-AS2, LOC100101148, LOC100101478, LOC100128006, LOC100128076, LOC100128164, LOC100128176, LOC100128233, LOC100128239, LOC100128317, LOC100128361, LOC100128554, LOC100128568, LOC100128573, LOC100128593, LOC100128714, LOC100128770, LOC100128885, LOC100128993, LOC100129027, LOC100129055, LOC100129083, LOC100129138, LOC100129175, LOC100129203, LOC100129307, LOC100129316, LOC100129345, LOC100129434, LOC100129518, LOC100129520, LOC100129534, LOC100129603, LOC100129617, LOC100129620, LOC100129636, LOC100129697, LOC100129924, LOC100129931, LOC100129935, LOC100129940, LOC100129973, LOC100130238, LOC100130264, LOC100130298, LOC100130370, LOC100130417, LOC100130451, LOC100130452, LOC100130476, LOC100130587, LOC100130673, LOC100130691, LOC100130700, LOC100130849, LOC100130880, LOC100130899, LOC100130950, LOC100130964, LOC100130992, LOC100131047, LOC100131107, LOC100131257, LOC100131289, LOC100131303, LOC100131315, LOC100131347, LOC100131496, LOC100131626, LOC100131635, LOC100132057, LOC100132062, LOC100132077, LOC100132078, LOC100132146, LOC100132174, LOC100132215, LOC100132304, LOC100132735, LOC100132741, LOC100132781, LOC100132831, LOC100133050, LOC100133077, LOC100133286, LOC100133315, LOC100133331, LOC100133461, LOC100133669, LOC100133920, LOC100134317, LOC100134368, LOC100134391, LOC100134868, LOC100144595, LOC100147773, LOC100190940, LOC100190986, LOC100192426, LOC100240728, LOC100240734, LOC100240735, LOC100271832, LOC100272216, LOC100287010, LOC100287042, LOC100287072, LOC100287225, LOC100287592, LOC100287632, LOC100287728, LOC100287792, LOC100287834, LOC100287846, LOC100287944, LOC100288069, LOC100288162, LOC100288181, LOC100288254, LOC100288637, LOC100288748, LOC100288866, LOC100288911, LOC100288966, LOC100289333, LOC100289473, LOC100289495, LOC100289580, LOC100289650, LOC100289656, LOC100289673, LOC100294362, LOC100303749, LOC100335030, LOC100420587, LOC100421746, LOC100422212, LOC100422556, LOC100499194, LOC100499484-C9ORF174, LOC100500773, LOC100505478, LOC100505515, LOC100505622, LOC100505625, LOC100505635, LOC100505658, LOC100505715, LOC100505716, LOC100505736, LOC100505782, LOC100505795, LOC100505817, LOC100505841, LOC100505878, LOC100505887, LOC100505912, LOC100505915, LOC100505918, LOC100505920, LOC100505921, LOC100505978, LOC100506023, LOC100506071, LOC100506076, LOC100506083, LOC100506085, LOC100506122, LOC100506123, LOC100506124, LOC100506127, LOC100506136, LOC100506159, LOC100506175, LOC100506188, LOC100506207, LOC100506258, LOC100506271, LOC100506272, LOC100506274, LOC100506281, LOC100506321, LOC100506368, LOC100506371, LOC100506384, LOC100506388, LOC100506393, LOC100506403, LOC100506406, LOC100506422, LOC100506444, LOC100506457, LOC100506470, LOC100506472, LOC100506474, LOC100506497, LOC100506526, LOC100506551, LOC100506585, LOC100506679, LOC100506682, LOC100506688, LOC100506700, LOC100506725, LOC100506747, LOC100506790, LOC100506801, LOC100506834, LOC100506858, LOC100506869, LOC100506895, LOC100507006, LOC100507053, LOC100507065, LOC100507073, LOC100507091, LOC100507140, LOC100507144, LOC100507156, LOC100507175, LOC100507194, LOC100507201, LOC100507205, LOC100507250, LOC100507283, LOC100507334, LOC100507346, LOC100507351, LOC100507373, LOC100507377, LOC100507384, LOC100507387, LOC100507388, LOC100507391, LOC100507406, LOC100507412, LOC100507431, LOC100507437, LOC100507443, LOC100507462, LOC100507468, LOC100507477, LOC100507506, LOC100507534, LOC100507537, LOC100507564, LOC100507600, LOC100507661, LOC100508046, LOC100631378, LOC100652931, LOC100652999, LOC100996249, LOC100996251, LOC100996255, LOC100996263, LOC100996286, LOC100996291, LOC100996324, LOC100996325, LOC100996338, LOC100996342, LOC100996349, LOC100996351, LOC100996385, LOC100996455, LOC100996583, LOC100996634, LOC100996635, LOC100996654, LOC100996671, LOC100996679, LOC100996693, LOC100996694, LOC101054525, LOC101059948, LOC101060019, LOC101060385, LOC101060498, LOC101060542, LOC101060553, LOC101241902, LOC101243545, LOC101409256, LOC101448202, LOC101593348, LOC101805491, LOC101926889, LOC101926892, LOC101926897, LOC101926898, LOC101926905, LOC101926908, LOC101926911, LOC101926913, LOC101926915, LOC101926935, LOC101926940, LOC101926941, LOC101926942, LOC101926943, LOC101926955, LOC101926960, LOC101926962, LOC101926963, LOC101926964, LOC101926966, LOC101926975, LOC101927020, LOC101927023, LOC101927040, LOC101927043, LOC101927048, LOC101927051, LOC101927053, LOC101927055, LOC101927058, LOC101927062, LOC101927066, LOC101927069, LOC101927070, LOC101927078, LOC101927079, LOC101927081, LOC101927082, LOC101927087, LOC101927120, LOC101927123, LOC101927124, LOC101927131, LOC101927132, LOC101927139, LOC101927142, LOC101927143, LOC101927153, LOC101927156, LOC101927157, LOC101927159, LOC101927164, LOC101927166, LOC101927179, LOC101927181, LOC101927188, LOC101927189, LOC101927190, LOC101927196, LOC101927207, LOC101927211, LOC101927229, LOC101927230, LOC101927237, LOC101927239, LOC101927243, LOC101927244, LOC101927248, LOC101927257, LOC101927267, LOC101927272, LOC101927274, LOC101927282, LOC101927284, LOC101927285, LOC101927286, LOC101927292, LOC101927296, LOC101927305, LOC101927310, LOC101927314, LOC101927318, LOC101927334, LOC101927342, LOC101927348, LOC101927356, LOC101927358, LOC101927359, LOC101927374, LOC101927378, LOC101927379, LOC101927394, LOC101927406, LOC101927410, LOC101927411, LOC101927412, LOC101927415, LOC101927416, LOC101927419, LOC101927421, LOC101927431, LOC101927434, LOC101927437, LOC101927438, LOC101927446, LOC101927450, LOC101927460, LOC101927464, LOC101927468, LOC101927472, LOC101927476, LOC101927478, LOC101927482, LOC101927488, LOC101927495, LOC101927497, LOC101927502, LOC101927523, LOC101927526, LOC101927539, LOC101927543, LOC101927549, LOC101927550, LOC101927557, LOC101927560, LOC101927572, LOC101927575, LOC101927577, LOC101927580, LOC101927583, LOC101927587, LOC101927588, LOC101927592, LOC101927604, LOC101927606, LOC101927616, LOC101927619, LOC101927620, LOC101927623, LOC101927636, LOC101927637, LOC101927640, LOC101927641, LOC101927650, LOC101927651, LOC101927653, LOC101927657, LOC101927661, LOC101927666, LOC101927668, LOC101927683, LOC101927691, LOC101927692, LOC101927694, LOC101927697, LOC101927701, LOC101927708, LOC101927709, LOC101927730, LOC101927735, LOC101927740, LOC101927762, LOC101927766, LOC101927768, LOC101927769, LOC101927770, LOC101927787, LOC101927795, LOC101927797, LOC101927798, LOC101927811, LOC101927814, LOC101927815, LOC101927817, LOC101927822, LOC101927827, LOC101927829, LOC101927835, LOC101927839, LOC101927844, LOC101927845, LOC101927847, LOC101927849, LOC101927865, LOC101927869, LOC101927870, LOC101927876, LOC101927881, LOC101927884, LOC101927901, LOC101927905, LOC101927907, LOC101927911, LOC101927914, LOC101927915, LOC101927919, LOC101927924, LOC101927926, LOC101927932, LOC101927948, LOC101927950, LOC101927954, LOC101927964, LOC101927967, LOC101927969, LOC101927973, LOC101927987, LOC101928002, LOC101928008, LOC101928009, LOC101928012, LOC101928030, LOC101928034, LOC101928035, LOC101928043, LOC101928048, LOC101928052, LOC101928053, LOC101928063, LOC101928068, LOC101928075, LOC101928093, LOC101928100, LOC101928103, LOC101928105, LOC101928107, LOC101928118, LOC101928128, LOC101928131, LOC101928134, LOC101928135, LOC101928137, LOC101928140, LOC101928150, LOC101928161, LOC101928162, LOC101928163, LOC101928167, LOC101928168, LOC101928174, LOC101928191, LOC101928201, LOC101928203, LOC101928205, LOC101928211, LOC101928222, LOC101928226, LOC101928227, LOC101928231, LOC101928233, LOC101928241, LOC101928253, LOC101928254, LOC101928259, LOC101928266, LOC101928269, LOC101928270, LOC101928272, LOC101928273, LOC101928279, LOC101928280, LOC101928283, LOC101928295, LOC101928298, LOC101928304, LOC101928306, LOC101928307, LOC101928314, LOC101928322, LOC101928324, LOC101928327, LOC101928333, LOC101928335, LOC101928336, LOC101928358, LOC101928371, LOC101928372, LOC101928381, LOC101928386, LOC101928398, LOC101928401, LOC101928404, LOC101928414, LOC101928416, LOC101928417, LOC101928418, LOC101928435, LOC101928436, LOC101928437, LOC101928438, LOC101928441, LOC101928443, LOC101928446, LOC101928449, LOC101928453, LOC101928460, LOC101928461, LOC101928471, LOC101928476, LOC101928489, LOC101928491, LOC101928495, LOC101928509, LOC101928514, LOC101928516, LOC101928517, LOC101928519, LOC101928523, LOC101928535, LOC101928539, LOC101928540, LOC101928551, LOC101928565, LOC101928567, LOC101928569, LOC101928580, LOC101928590, LOC101928597, LOC101928600, LOC101928618, LOC101928622, LOC101928626, LOC101928650, LOC101928651, LOC101928661, LOC101928663, LOC101928673, LOC101928674, LOC101928682, LOC101928694, LOC101928696, LOC101928697, LOC101928700, LOC101928708, LOC101928710, LOC101928718, LOC101928730, LOC101928731, LOC101928737, LOC101928738, LOC101928739, LOC101928748, LOC101928751, LOC101928766, LOC101928769, LOC101928775, LOC101928778, LOC101928782, LOC101928786, LOC101928790, LOC101928796, LOC101928797, LOC101928804, LOC101928809, LOC101928812, LOC101928823, LOC101928834, LOC101928841, LOC101928844, LOC101928847, LOC101928851, LOC101928855, LOC101928858, LOC101928861, LOC101928865, LOC101928880, LOC101928882, LOC101928886, LOC101928891, LOC101928894, LOC101928896, LOC101928909, LOC101928911, LOC101928936, LOC101928937, LOC101928940, LOC101928942, LOC101928943, LOC101928944, LOC101928961, LOC101928973, LOC101928977, LOC101928978, LOC101928979, LOC101928988, LOC101928989, LOC101928992, LOC101928994, LOC101928995, LOC101929019, LOC101929023, LOC101929034, LOC101929057, LOC101929058, LOC101929064, LOC101929073, LOC101929076, LOC101929080, LOC101929084, LOC101929095, LOC101929099, LOC101929106, LOC101929116, LOC101929122, LOC101929123, LOC101929124, LOC101929125, LOC101929140, LOC101929144, LOC101929148, LOC101929153, LOC101929154, LOC101929162, LOC101929164, LOC101929172, LOC101929181, LOC101929194, LOC101929199, LOC101929207, LOC101929210, LOC101929217, LOC101929224, LOC101929234, LOC101929237, LOC101929239, LOC101929241, LOC101929259, LOC101929260, LOC101929268, LOC101929279, LOC101929282, LOC101929284, LOC101929294, LOC101929295, LOC101929297, LOC101929312, LOC101929315, LOC101929319, LOC101929331, LOC101929337, LOC101929340, LOC101929353, LOC101929371, LOC101929374, LOC101929378, LOC101929380, LOC101929384, LOC101929395, LOC101929406, LOC101929412, LOC101929413, LOC101929415, LOC101929420, LOC101929431, LOC101929439, LOC101929441, LOC101929450, LOC101929452, LOC101929454, LOC101929464, LOC101929468, LOC101929470, LOC101929473, LOC101929486, LOC101929488, LOC101929497, LOC101929504, LOC101929505, LOC101929512, LOC101929517, LOC101929526, LOC101929528, LOC101929529, LOC101929532, LOC101929549, LOC101929550, LOC101929551, LOC101929555, LOC101929563, LOC101929565, LOC101929567, LOC101929570, LOC101929577, LOC101929579, LOC101929584, LOC101929586, LOC101929592, LOC101929595, LOC101929596, LOC101929607, LOC101929608, LOC101929622, LOC101929625, LOC101929631, LOC101929645, LOC101929646, LOC101929653, LOC101929657, LOC101929660, LOC101929662, LOC101929679, LOC101929680, LOC101929681, LOC101929694, LOC101929696, LOC101929698, LOC101929705, LOC101929710, LOC101929717, LOC101929719, LOC101929721, LOC101929723, LOC101929733, LOC101929741, LOC101929753, LOC101929754, LOC101929762, LOC101929897, LOC101930010, LOC101930452, LOC102467079, LOC102467081, LOC102467147, LOC102467212, LOC102467213, LOC102467214, LOC102467216, LOC102467217, LOC102467222, LOC102467223, LOC102467224, LOC102467225, LOC102467226, LOC102467655, LOC102477328, LOC102503427, LOC102546226, LOC102546228, LOC102546229, LOC102546294, LOC102546299, LOC102577426, LOC102606466, LOC102723330, LOC102723344, LOC102723362, LOC102723373, LOC102723376, LOC102723377, LOC102723427, LOC102723439, LOC102723448, LOC102723505, LOC102723517, LOC102723544, LOC102723582, LOC102723604, LOC102723617, LOC102723649, LOC102723661, LOC102723701, LOC102723704, LOC102723709, LOC102723769, LOC102723778, LOC102723824, LOC102723828, LOC102723831, LOC102723833, LOC102723854, LOC102723883, LOC102723885, LOC102723895, LOC102723968, LOC102724000, LOC102724020, LOC102724034, LOC102724053, LOC102724058, LOC102724096, LOC102724163, LOC102724188, LOC102724190, LOC102724201, LOC102724238, LOC102724279, LOC102724297, LOC102724301, LOC102724321, LOC102724323, LOC102724421, LOC102724434, LOC102724467, LOC102724511, LOC102724555, LOC102724596, LOC102724604, LOC102724612, LOC102724623, LOC102724651, LOC102724659, LOC102724661, LOC102724663, LOC102724699, LOC102724710, LOC102724719, LOC102724776, LOC102724804, LOC102724849, LOC102724890, LOC102724913, LOC102724927, LOC102724933, LOC102724957, LOC102724958, LOC102725079, LOC102800447, LOC103091866, LOC103171574, LOC103191607, LOC103312105, LOC103352541, LOC103908605, LOC104054148, LOC104613533, LOC105274304, LOC105370306, LOC105372441, LOC105373300, LOC105376360, LOC105376671, LOC105378732, LOC105447645, LOC105447648, LOC105616981, LOC105667213, LOC105748977, LOC105755953, LOC143666, LOC145474, LOC145845, LOC146513, LOC148696, LOC148709, LOC149373, LOC149684, LOC149950, LOC150935, LOC151121, LOC151174, LOC151475, LOC151484, LOC152578, LOC153684, LOC154449, LOC154761, LOC157273, LOC158434, LOC158435, LOC1720, LOC200726, LOC200772, LOC202181, LOC221122, LOC221946, LOC253573, LOC254028, LOC254896, LOC255187, LOC255654, LOC256880, LOC283038, LOC283045, LOC283140, LOC283177, LOC283194, LOC283214, LOC283299, LOC283332, LOC283352, LOC283440, LOC283575, LOC283585, LOC283683, LOC283710, LOC283731, LOC283856, LOC284023, LOC284080, LOC284294, LOC284379, LOC284395, LOC284412, LOC284578, LOC284581, LOC284632, LOC284648, LOC284661, LOC284788, LOC284798, LOC284825, LOC284865, LOC284930, LOC284933, LOC284950, LOC285000, LOC285043, LOC285484, LOC285593, LOC285626, LOC285627, LOC285629, LOC285692, LOC285696, LOC285740, LOC285762, LOC285766, LOC285768, LOC285804, LOC285819, LOC285847, LOC285889, LOC286083, LOC286114, LOC286177, LOC286238, LOC286297, LOC286359, LOC286370, LOC338694, LOC338797, LOC338963, LOC339059, LOC339166, LOC339260, LOC339298, LOC339529, LOC339539, LOC339568, LOC339593, LOC339622, LOC339666, LOC339807, LOC339862, LOC339874, LOC339975, LOC340017, LOC340074, LOC340107, LOC340113, LOC340357, LOC340512, LOC343052, LOC344967, LOC349160, LOC374443, LOC375196, LOC387810, LOC388242, LOC388406, LOC388436, LOC388780, LOC388813, LOC388849, LOC388882, LOC388942, LOC389033, LOC389199, LOC389247, LOC389705, LOC389831, LOC389834, LOC389895, LOC390705, LOC391003, LOC391322, LOC392196, LOC392232, LOC392364, LOC392452, LOC399715, LOC399815, LOC399886, LOC400043, LOC400541, LOC400548, LOC400553, LOC400558, LOC400620, LOC400655, LOC400661, LOC400684, LOC400685, LOC400706, LOC400736, LOC400794, LOC400867, LOC400940, LOC400997, LOC401052, LOC401134, LOC401177, LOC401242, LOC401286, LOC401312, LOC401324, LOC401357, LOC401463, LOC401557, LOC401585, LOC403312, LOC403323, LOC414300, LOC439933, LOC439994, LOC440028, LOC440040, LOC440117, LOC440173, LOC440300, LOC440390, LOC440446, LOC440461, LOC440600, LOC440602, LOC440700, LOC440704, LOC440895, LOC440896, LOC440910, LOC440982, LOC441025, LOC441081, LOC441155, LOC441178, LOC441204, LOC441242, LOC441454, LOC441455, LOC441601, LOC441666, LOC442028, LOC442132, LOC442497, LOC494127, LOC494141, LOC51145, LOC541472, LOC541473, LOC550113, LOC55338, LOC554223, LOC554249, LOC574538, LOC606724, LOC613037, LOC613266, LOC63930, LOC641367, LOC641746, LOC642366, LOC642423, LOC642426, LOC642929, LOC642943, LOC643201, LOC643339, LOC643406, LOC643441, LOC643542, LOC643623, LOC643711, LOC643733, LOC643770, LOC643802, LOC643923, LOC644145, LOC644189, LOC644285, LOC644554, LOC644669, LOC644838, LOC644919, LOC645166, LOC645382, LOC645434, LOC645752, LOC645949, LOC646029, LOC646268, LOC646522, LOC646736, LOC646743, LOC646813, LOC646938, LOC647323, LOC647859, LOC649133, LOC649324, LOC649352, LOC650226, LOC650293, LOC653513, LOC653602, LOC653786, LOC654841, LOC727924, LOC728084, LOC728095, LOC728175, LOC728392, LOC728485, LOC728673, LOC728989, LOC729080, LOC729083, LOC729159, LOC729296, LOC729348, LOC729506, LOC729609, LOC729658, LOC729683, LOC729732, LOC729737, LOC729930, LOC729950, LOC729966, LOC729987, LOC730159, LOC730668, LOC731157, LOC731424, LOC79160, LOC79999, LOC90246, LOC90768, LOC91450, LOH12CR2, LOR, LOXHD1, LOXL1, LOXL1-AS1, LOXL3, LPAR1, LPAR3, LPAR4, LPAR5, LPCAT2, LPL, LPO, LPP-AS1, LPPR1, LPPR3, LPPR4, LPPR5, LRAT, LRCH2, LRFN1, LRFN2, LRFN5, LRGUK, LRIT1, LRIT2, LRMP, LRP1-AS, LRP1B, LRP2, LRP2BP, LRP4, LRP4-AS1, LRRC10, LRRC10B, LRRC14B, LRRC16B, LRRC17, LRRC18, LRRC19, LRRC2-AS1, LRRC26, LRRC3-AS1, LRRC30, LRRC34, LRRC36, LRRC37A11P, LRRC37A2, LRRC37A4P, LRRC37A5P, LRRC37A6P, LRRC37A8P, LRRC38, LRRC3B, LRRC4, LRRC43, LRRC46, LRRC48, LRRC4B, LRRC4C, LRRC52, LRRC55, LRRC6, LRRC63, LRRC66, LRRC7, LRRC70, LRRC71, LRRC72, LRRC73, LRRC74A, LRRC74B, LRRC75A, LRRC9, LRRD1, LRRIQ1, LRRIQ3, LRRIQ4, LRRK1, LRRN1, LRRN3, LRRN4, LRRN4CL, LRRTM1, LRRTM2, LRRTM3, LRRTM4, LRTM1, LRTM2, LSAMP, LSAMP-AS1, LSMEM2, LSP1P3, LST1, LTBP2, LTF, LTK, LUM, LUNAR1, LURAP1, LUZP2, LUZP4, LVCAT1, LVCAT5, LVRN, LXN, LY6D, LY6G6C, LY6G6E, LY6H, LY6K, LY86, LY86-AS1, LY9, LYG2, LYPD2, LYPD3, LYPD4, LYPD5, LYPD6, LYPD6B, LYPD8, LYVE1, LYZL1, LYZL2, LYZL4, LYZL6, LZTS1, LZTS1-AS1, M1AP, MAB21L1, MAB21L3, MACC1, MACC1-AS1, MACROD2, MACROD2-AS1, MACROD2-IT1, MADCAM1, MAEL, MAFA-AS1, MAFTRR, MAG, MAGEA1, MAGEA10, MAGEA11, MAGEA12, MAGEA2B, MAGEA3, MAGEA4, MAGEA5, MAGEA6, MAGEA8, MAGEA8-AS1, MAGEA9B, MAGEB1, MAGEB10, MAGEB16, MAGEB17, MAGEB18, MAGEB2, MAGEB3, MAGEB4, MAGEB5, MAGEB6, MAGEC1, MAGEC2, MAGEC3, MAGED4, MAGED4B, MAGEE2, MAGEL2, MAGI1-AS1, MAGI2, MAGI2-AS2, MAK, MAL, MALL, MALRD1, MAMDC2, MAMDC2-AS1, MAML2, MAMSTR, MAN1C1, MANSC4, MAP1A, MAP1B, MAP1LC3C, MAP3K12, MAP3K15, MAP3K19, MAP4K1, MAP6, MAP9, MAPK10, MAPK15, MAPT-AS1, MAPT-IT1, MARCH10, MARCH11, MARCH4, MARCO, MARK1, MARK2P9, MAS1, MAS1L, MAST1, MATK, MATN1, MATN3, MB21D2, MBD3L1, MBD3L2, MBD3L3, MBD3L4, MBD3L5, MBNL1-AS1, MBOAT2, MBOAT4, MC2R, MC3R, MC4R, MCCD1, MCEMP1, MCF2, MCF2L2, MCHR2, MCHR2-AS1, MCIDAS, MCM8-AS1, MCMDC2, MCOLN2, MCOLN3, MCPH1-AS1, MCTP1, MCTP2, MDGA2, MDH1B, MDS2, MEAT6, MECOM, MED12L, MED15P9, MEDAG, MEF2C, MEF2C-AS1, MEFV, MEG3, MEG8, MEG9, MEGF10, MEGF11, MEI1, MEI4, MEIG1, MEIKIN, MEIOB, MEIS1-AS2, MEIS1-AS3, MEIS3, MEIS3P1, MEOX1, MEOX2, MEOX2-AS1, MEP1A, MEP1B, MEPE, MESP2, MESTIT1, METTL11B, METTL21C, METTL21EP, METTL24, MFAP2, MFAP4, MFAP5, MFSD2B, MFSD4, MFSD6L, MGAT3, MGAT3-AS1, MGAT4C, MGAT4D, MGAT4EP, MGAT5B, MGC15885, MGC16025, MGC27382, MGC2889, MGC32805, MGC34796, MGC39584, MGC45922, MGC57346-CRHR1, MGP, MHRT, MIAT, MILR1, MIMT1, MINOS1P1, MIOX, MIP, MIPEPP3, MISP, MIXL1, MKI67, MKNK1-AS1, MKRN20S, MKRN3, MKRN7P, MKX-AS1, MLANA, MLC1, MLF1, MLIP-IT1, MLLT4-AS1, MLN, MLNR, MMD2, MMEL1, MMP13, MMP16, MMP17, MMP2, MMP20, MMP21, MMP23A, MMP23B, MMP26, MMP27, MMP28, MMP8, MMRN1, MMRN2, MMS22L, MND1, MNDA, MNS1, MNX1, MNX1-AS1, MOBP, MOG, MORC1, MORF4L2-AS1, MORN3, MORN5, MOS, MOV10L1, MOXD1, MOXD2P, MPEG1, MPL, MPO, MPP2, MPP4, MPPED2, MRAP2, MRC1, MRC2, MRGPRD, MRGPRE, MRGPRF, MRGPRF-AS1, MRGPRG, MRGPRG-AS1, MRGPRX1, MRGPRX2, MRGPRX3, MRGPRX4, MRLN, MRO, MROH2B, MROH5, MROH7, MROH7-TTC4, MROH9, MRPL23-AS1, MRS2P2, MRVI1, MRVI1-AS1, MS4A1, MS4A10, MS4A12, MS4A13, MS4A14, MS4A15, MS4A18, MS4A2, MS4A3, MS4A4A, MS4A5, MS4A6A, MS4A6E, MS4A7, MS4A8, MSGN1, MSI1, MSLN, MSMB, MSR1, MSTN, MSTO2P, MSX2, MSX2P1, MT3, MTHFD2P1, MTL5, MTMR8, MTMR9LP, MTNR1A, MTNR1B, MTOR-AS1, MTRNR2L4, MTRNR2L5, MTRNR2L7, MTUS2, MTUS2-AS1, MTVR2, MUC1, MUC12, MUC15, MUC16, MUC17, MUC19, MUC2, MUC21, MUC22, MUC4, MUC5AC, MUC5B, MUC6, MUC7, MUCL1, MUSK, MX2, MXRA5, MYADML, MYADML2, MYB, MYBL2, MYBPC1, MYBPC2, MYBPC3, MYBPH, MYBPHL, MYCBP2-AS1, MYCBPAP, MYCN, MYCNOS, MYCNUT, MYCT1, MYEF2, MYEOV, MYF5, MYF6, MYH1, MYH11, MYH13, MYH15, MYH2, MYH4, MYH6, MYH7, MYH7B, MYH8, MYHAS, MYL1, MYL10, MYL2, MYL3, MYL4, MYL7, MYLK-AS2, MYLK2, MYLK3, MYLK4, MYO15A, MYO16, MYO16-AS1, MYO18B, MYO1A, MYO1F, MYO1G, MYO1H, MYO3A, MYO3B, MYO7B, MYOC, MYOCD, MYOD1, MYOG, MYOM2, MYOM3, MYOT, MYOZ1, MYOZ2, MYOZ3, MYRFL, MYT1, MYT1L, MYT1L-AS1, MYZAP, MZB1, N4BP3, NAA11, NAALAD2, NAALADL1, NAALADL2, NAALADL2-AS1, NAALADL2-AS2, NAALADL2-AS3, NACAD, NACAP1, NAGPA-AS1, NAIP, NALCN, NALCN-AS1, NAMA, NANOG, NANOGNB, NANOS2, NANOS3, NAP1L3, NAP1L6, NAPSB, NARR, NAT16, NAT8L, NAV2-AS2, NAV2-AS5, NBAT1, NBEA, NBEAP1, NBPF11, NBPF14, NBPF20, NBPF22P, NBPF4, NBPF6, NBPF7, NCAM1, NCAM1-AS1, NCAM2, NCAN, NCAPG, NCF1, NCF1B, NCF1C, NCF4, NCKAP1L, NCMAP, NCOA7-AS1, NCOR1P1, NCR1, NCR2, NCR3, NCRNAOC250, NCRUPAR, NDC80, NDFIP2-AS1, NDN, NDNF, NDP, NDST3, NDST4, NDUFA6-AS1, NDUFAF4P1, NEBL-AS1, NECAB1, NEFH, NEFM, NEGR1, NEGR1-IT1, NEIL3, NEK10, NEK2, NEK5, NELL1, NELL2, NES, NET01, NEU2, NEURL1, NEURL1-AS1, NEUROD1, NEUROD2, NEUROD4, NEUROD6, NEUROG1, NEUROG2, NEUROG3, NF1P2, NFAM1, NFE2, NFIA-AS1, NFIA-AS2, NGB, NGF, NGFR, NHEG1, NHLH1, NHLRC4, NHS, NHS-AS1, NHSL2, NID2, NIM1K, NIPAL4, NIPSNAP3B, NKAIN2, NKAIN3, NKAIN4, NKAPL, NKAPP1, NKD2, NKILA, NKPD1, NKX1-1, NKX1-2, NKX2-1, NKX2-1-AS1, NKX2-2, NKX2-3, NKX2-4, NKX2-5, NKX2-6, NKX2-8, NKX3-2, NKX6-1, NKX6-2, NKX6-3, NLGN1, NLGN1-AS1, NLGN3, NLGN4X, NLGN4Y, NLGN4Y-AS1, NLRC3, NLRC4, NLRP1, NLRP10, NLRP11, NLRP12, NLRP13, NLRP14, NLRP2, NLRP3, NLRP4, NLRP5, NLRP6, NLRP7, NLRP8, NLRP9, NMBR, NME5, NME8, NME9, NMNAT2, NMRK2, NMS, NMU, NMUR1, NMUR2, NOBOX, NOG, NOL4, NOS1, NOS3, NOSTRIN, NOTCH4, NOTO, NOVA1, NOVA2, NOX1, NOX3, NOX4, NOX5, NOXRED1, NPAP1, NPAS1, NPAS3, NPAS4, NPBWR2, NPFFR1, NPFFR2, NPHP1, NPHP3-AS1, NPHS1, NPHS2, NPIPA3, NPIPA5, NPIPA7, NPIPA8, NPIPB11, NPIPB4, NPIPB5, NPIPB6, NPIPB8, NPIPB9, NPNT, NPS, NPSR1, NPSR1-AS1, NPVF, NPY, NPY1R, NPY2R, NPY4R, NPY5R, NPY6R, NROB1, NR2E1, NR2E3, NR2F1-AS1, NR2F2-AS1, NR5A1, NRADDP, NREP-AS1, NRG1-IT1, NRG1-IT3, NRG2, NRG3, NRG3-AS1, NRGN, NRIP2, NRIP3, NRK, NRL, NRN1, NRON, NRROS, NRSN1, NRXN1, NRXN2, NRXN3, NSFP1, NSG1, NSUN7, NT5C1A, NT5C1B, NT5C1B-RDH14, NTF4, NTM, NTM-IT, NTN3, NTN5, NTNG1, NTNG2, NTRK1, NTRK2, NTRK3, NTRK3-AS1, NTS, NTSR1, NTSR2, NUCB1-AS1, NUDT10, NUDT11, NUDT4P2, NUDT9P1, NUF2, NUP210L, NUP210P1, NUP62CL, NUPR1L, NUTM1, NUTM2A, NUTM2B, NUTM2D, NUTM2F, NUTM2G, NWD1, NWD2, NXF2B, NXF3, NXF4, NXF5, NXNL1, NXNL2, NXPE1, NXPE2, NXPE3, NXPE4, NXPH1, NXPH2, NXPH3, NYAP1, NYAP2, NYX, OACYLP, OBP2A, OBP2B, OBSCN, OC90, OCA2, OCM, OCM2, OCSTAMP, ODAM, ODF1, ODF3, ODF3L1, ODF3L2, ODF4, OGN, OLAH, OLFM1, OLFM3, OLFM4, OLFML1, OLFML2A, OLFML2B, OLFML3, 0LIG1, 0LIG2, 0LIG3, OLR1, OMD, OMG, ONECUT3, OOSP2, OPA1-AS1, OPALIN, OPCML, OPHN1, OPN1LW, OPN1MW2, OPN1SW, OPN4, OPN5, OPRD1, OPRK1, OPRL1, OPRM1, OPTC, OR10A2, OR10A3, OR10A4, OR10A5, OR10A6, OR10A7, OR10AD1, OR10AG1, OR10C1, OR10G2, OR10G3, OR10G4, OR10G7, OR10G8, OR10G9, OR10H1, OR10H2, OR10H3, OR10H4, OR10H5, OR10J1, OR10J3, OR10J5, OR10K1, OR10K2, OR10P1, OR10Q1, OR1OR2, OR10S1, OR10T2, OR10V1, OR10V2P, OR10W1, OR10X1, OR10Z1, OR11A1, OR11G2, OR11H1, OR11H12, OR11H2, OR11H4, OR11H6, OR11L1, OR12D2, OR12D3, OR13A1, OR13C2, OR13C3, OR13C4, OR13C5, OR13C8, OR13C9, OR13D1, OR13F1, OR13G1, OR13H1, OR13J1, OR14A16, OR14C36, OR14I1, OR14J1, OR1A1, OR1A2, OR1B1, OR1C1, OR1D2, OR1D4, OR1D5, OR1E1, OR1E2, OR1F1, OR1F2P, OR1G1, OR1I1, OR1J1, OR1J2, OR1J4, OR1K1, OR1L1, OR1L3, OR1L4, OR1L6, OR1L8, OR1M1, OR1N1, OR1N2, OR1Q1, OR1S1, OR1S2, OR2A1-AS1, OR2A12, OR2A14, OR2A2, OR2A20P, OR2A25, OR2A4, OR2A42, OR2A5, OR2A9P, OR2AE1, OR2AG1, OR2AG2, OR2AK2, OR2AP1, OR2AT4, OR2B11, OR2B2, OR2B3, OR2C1, OR2C3, OR2D2, OR2D3, OR2F1, OR2F2, OR2G2, OR2G3, OR2G6, OR2H1, OR2H2, OR2J2, OR2J3, OR2K2, OR2L13, OR2L1P, OR2L2, OR2L3, OR2L5, OR2L8, OR2M1P, OR2M2, OR2M3, OR2M4, OR2M5, OR2M7, OR2S2, OR2T1, OR2T10, OR2T11, OR2T12, OR2T2, OR2T27, OR2T29, OR2T3, OR2T33, OR2T34, OR2T35, OR2T4, OR2T5, OR2T6, OR2T8, OR2V1, OR2V2, OR2W1, OR2W3, OR2W5, OR2Y1, OR2Z1, OR3A1, OR3A2, OR3A3, OR3A4P, OR4A15, OR4A16, OR4A47, OR4A5, OR4B1, OR4C11, OR4C12, OR4C13, OR4C15, OR4C16, OR4C3, OR4C45, OR4C46, OR4C6, OR4D1, OR4D10, OR4D11, OR4D2, OR4D5, OR4D6, OR4D9, OR4E2, OR4F13P, OR4F15, OR4F17, OR4F21, OR4F4, OR4F5, OR4F6, OR4K1, OR4K13, OR4K14, OR4K15, OR4K17, OR4K2, OR4K5, OR4L1, OR4M1, OR4M2, OR4N2, OR4N3P, OR4N4, OR4N5, OR4P4, OR4Q3, OR4S1, OR4S2, OR4X1, OR4X2, OR51A2, OR51A4, OR51A7, OR51B2, OR51B4, OR51B5, OR51B6, OR51D1, OR51E1, OR51E2, OR51F1, OR51F2, OR51G1, OR51G2, OR51I1, OR51I2, OR51L1, OR51M1, OR51Q1, OR51S1, OR51T1, OR51V1, OR52A1, OR52A5, OR52B2, OR52B4, OR52B6, OR52D1, OR52E2, OR52E4, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR56A1, OR56A3, OR56A4, OR56A5, OR56B1, OR56B4, OR5A1, OR5A2, OR5AC2, OR5AK2, OR5AK4P, OR5AN1, OR5AP2, OR5AR1, OR5AS1, OR5B12, OR5B17, OR5B2, OR5B21, OR5B3, OR5C1, OR5D13, OR5D14, OR5D16, OR5D18, OR5E1P, OR5F1, OR5H1, OR5H14, OR5H15, OR5H2, OR5H6, OR5I1, OR5J2, OR5K1, OR5K2, OR5K3, OR5K4, OR5L1, OR5L2, OR5M1, OR5M10, OR5M11, OR5M3, OR5M8, OR5M9, OR5P2, OR5P3, OR5R1, OR5T1, OR5T2, OR5T3, OR5V1, OR5W2, OR6A2, OR6B1, OR6B2, OR6B3, OR6C1, OR6C2, OR6C3, OR6C4, OR6C6, OR6C65, OR6C68, OR6C70, OR6C74, OR6C75, OR6C76, OR6F1, OR6K2, OR6K3, OR6K6, OR6M1, OR6N1, OR6N2, OR6P1, OR6Q1, OR6S1, OR6T1, OR6V1, OR6W1P, OR6X1, OR6Y1, OR7A10, OR7A17, OR7A5, OR7C1, OR7C2, OR7D2, OR7D4, OR7E12P, OR7E156P, OR7E24, OR7E2P, OR7E37P, OR7E47P, OR7E5P, OR7E91P, OR7G1, OR7G2, OR7G3, OR8A1, OR8B12, OR8B2, OR8B3, OR8B4, OR8B8, OR8D1, OR8D2, OR8D4, OR8G1, OR8G2, OR8G5, OR8H1, OR8H2, OR8H3, OR8I2, OR8J1, OR8J3, OR8K1, OR8K3, OR8K5, OR8S1, OR8U1, OR8U8, OR9A2, OR9A4, OR9G1, OR9G4, OR9I1, OR9K2, OR9Q1, OR9Q2, ORAI2, ORC1, OSBP2, OSBPL10, OSBPL10-AS1, OSBPL5, OSBPL7, OSCAR, OSGEPL1-AS1, OSM, OSMR-AS1, OSR2, OSTN, OTOA, OTOF, OTOG, OTOGL, OTOL1, OTOP1, OTOP2, OTOP3, OTOR, OTOS, OTUD6A, OTUD7A, OTX1, OTX2, OTX2-AS1, OVAAL, OVCH1, OVCH1-AS1, OVCH2, OVOL1-AS1, OVOL2, OVOL3, OXCT1, OXCT1-AS1, OXCT2, OXGR1, P2RX1, P2RX2, P2RX3, P2RX5-TAX1BP3, P2RX6, P2RY1, P2RY10, P2RY12, P2RY13, P2RY14, P2RY4, P2RY8, P3H2-AS1, P3H3, PABPC1L2A, PABPC1L2B, PABPC1L2B-AS1, PABPC1P2, PABPC4L, PABPC5, PABPC5-AS1, PABPN1L, PACERR, PACRG, PACRG-AS1, PACSIN1, PADI2, PADI3, PADI4, PADI6, PAEP, PAGE1, PAGE2, PAGE2B, PAGE3, PAGE4, PAGE5, PAK7, PALD1, PALM, PAMR1, PANX3, PAPL, PAPOLB, PAPPA, PAPPA-AS1, PAPPA2, PAQR8, PARM1, PART1, PARVG, PASD1, PATE1, PATE2, PATE3, PATE4, PAUPAR, PAX1, PAX2, PAX3, PAX4, PAX5, PAX6, PAX7, PAX9, PBK, PBOV1, PBX4, PCA3, PCAT1, PCAT18, PCAT19, PCAT29, PCAT4, PCAT5, PCBP3, PCCA-AS1, PCDH10, PCDH11X, PCDH11Y, PCDH12, PCDH15, PCDH17, PCDH18, PCDH19, PCDH7, PCDH8, PCDH9-AS3, PCDH9-AS4, PCDHA10, PCDHA11, PCDHAl2, PCDHA13, PCDHA2, PCDHA3, PCDHA4, PCDHA5, PCDHA6, PCDHA7, PCDHA8, PCDHA9, PCDHB1, PCDHB10, PCDHB11, PCDHB12, PCDHB13, PCDHB14, PCDHB15, PCDHB16, PCDHB17P, PCDHB18P, PCDHB19P, PCDHB2, PCDHB3, PCDHB4, PCDHB5, PCDHB6, PCDHB7, PCDHB8, PCDHB9, PCDHGA1, PCDHGA11, PCDHGA12, PCDHGA2, PCDHGA3, PCDHGA4, PCDHGA5, PCDHGA6, PCDHGA7, PCDHGA8, PCDHGA9, PCDHGB1, PCDHGB2, PCDHGB3, PCDHGB7, PCDHGB8P, PCDHGC4, PCDHGC5, PCED1B-AS1, PCGEM1, POLO, PCNAP1, PCNXL2, PCOLCE-AS1, PCP4, PCSK1, PCSK1N, PCSK2, PCYT1B, PCYT1B-AS1, PDC, PDCD1, PDCD1LG2, PDCD6IPP2, PDCL2, PDE10A, PDE1A, PDE1C, PDE3A, PDE6A, PDE6B, PDE6C, PDE6G, PDE6H, PDE7B, PDE8B, PDGFD, PDHA2, PDIA2, PDILT, PDLIM4, PDPN, PDX1, PDX1-AS1, PDYN, PDZD2, PDZD3, PDZD4, PDZD7, PDZD9, PDZK1P1, PDZRN3, PDZRN3-AS1, PDZRN4, PEBP4, PECAM1, PEG3-AS1, PENK, PER4, PEX5L, PF4, PFN1P2, PFN3, PFN4, PCA3, PGA4, PGA5, PGAM1P5, PGAM4, PGBD3, PGC, PGCP1, PGK2, PGLYRP1, PGLYRP3, PGLYRP4, PGM5, PGM5-AS1, PGM5P2, PGM5P3-AS1, PGM5P4-AS1, PGPEP1L, PGR, PHACTR1, PHACTR3, PHC1, PHEX-AS1, PHF21B, PHF24, PHGR1, PHKA2-AS1, PHOSPHO1, PHOX2A, PHOX2B, PI15, PI16, PI4KAP1, PIANP, PIF1, PIFO, PIH1D3, PIK3CD-AS1, PIK3CG, PIK3R5, PIK3R6, PILRA, PIN1P1, PINLYP, PIP, PIP5K1B, PIP5K1P1, PIRT, PISRT1, PITPNM3, PITX1, PITX2, PITX3, PIWIL1, PIWIL3, PIWIL4, PKD1L1, PKD1L3, PKD2L2, PKDREJ, PKHD1L1, PKIA, PKIA-AS1, PKN2-AS1, PKNOX2, PKNOX2-AS1, PKP1, PKP3, PLA2G10, PLA2G1B, PLA2G2C, PLA2G2D, PLA2G2E, PLA2G2F, PLA2G3, PLA2G4A, PLA2G4D, PLA2G4E, PLA2G4E-AS1, PLA2G4F, PLA2G5, PLA2G7, PLA2R1, PLAC1, PLAC4, PLAC8, PLAC9, PLAG1, PLAGL1, PLAT, PLB1, PLCB2, PLCB4, PLCE1-AS1, PLCE1-AS2, PLCG1-AS1, PLCH1, PLCH2, PLCL1, PLCXD2-AS1, PLCXD3, PLCZ1, PLD4, PLD5, PLEK, PLEKHB1, PLEKHD1, PLEKHG4B, PLEKHG7, PLEKHH2, PLEKHS1, PLET1, PLGLB2, PLIN1, PLK4, PLK5, PLN, PLP1, PLSCR2, PLSCR5, PLTP, PLXDC1, PLXDC2, PLXNA4, PM20D1, PMCH, PMCHL1, PMCHL2, PMFBP1, PMP2, PMS2P5, PMS2P7, PMS2P9, PNCK, PNLIP, PNLIPRP1, PNLIPRP2, PNLIPRP3, PNMA2, PNMA3, PNMA5, PNMAL1, PNMAL2, PNMT, PNOC, PNPLA1, PODN, PODNL1, POLN, POLQ, POLR2J4, POM121L10P, POM121L12, POM121L1P, POM121L2, POM121L4P, POM121L8P, POM121L9P, POMC, POMZP3, POPDC3, POSTN, POT1-AS1, POTEA, POTEB, POTEB3, POTEC, POTED, POTEE, POTEF, POTEG, POTEH, POTEH-AS1, POTEI, POTEJ, POTEKP, POTEM, POU1F1, POU2AF1, POU2F2, POU2F3, POU3F2, POU3F3, POU3F4, POU4F1, POU4F2, POU4F3, POU5F1B, POU5F1P3, POU5F1P4, POU5F1P5, POU5F2, POU6F2, PP12613, PP14571, PP2D1, PPAP2C, PPAPDC1A, PPAPDC3, PPBP, PPBPP2, PPEF1, PPEF1-AS1, PPEF2, PPFIA2, PPIAL4A, PPIAL4C, PPIAL4E, PPIAL4F, PPIAL4G, PPIAP30, PPIL6, PPM1E, PPM1N, PPP1R14A, PPP1R14D, PPP1R16B, PPP1R17, PPP1R1B, PPP1R2P3, PPP1R2P9, PPP1R36, PPP1R3A, PPP1R3F, PPP1R42, PPP2R2B, PPP2R2B-IT1, PPP3R2, PPP4R1-AS1, PPP4R3CP, PPP5D1, PPY, PPY2P, PQLC2L, PRAC1, PRAC2, PRAM1, PRAME, PRAMEF1, PRAMEF10, PRAMEF11, PRAMEF12, PRAMEF14, PRAMEF15, PRAMEF17, PRAMEF19, PRAMEF2, PRAMEF20, PRAMEF22, PRAMEF26, PRAMEF4, PRAMEF5, PRAMEF6, PRAMEF7, PRAMEF8, PRB1, PRB2, PRB3, PRB4, PRC1-AS1, PRCAT47, PROD, PRDM1, PRDM11, PRDM12, PRDM13, PRDM14, PRDM16, PRDM5, PRDM6, PRDM7, PRDM8, PRDM9, PRELP, PREX2, PRF1, PRG1, PRG3, PRH1-PRR4, PRH2, PRICKLE1, PRICKLE2-AS1, PRICKLE2-AS2, PRICKLE2-AS3, PRIMA1, PRKACG, PRKAG3, PRKAR2A-AS1, PRKAR2B, PRKCA-AS1, PRKCB, PRKCG, PRKCQ, PRKD1, PRKG1, PRKG1-AS1, PRKG2, PRKX-AS1, PRKXP1, PRKY, PRL, PRLH, PRM1, PRM2, PRM3, PRMTS-AS1, PRMT8, PRNCR1, PRND, PRNT, PRO1804, PROB1, PROCA1, PROK1, PROK2, PROKR1, PROKR2, PROL1, PROM1, PROM2, PROP1, PRORY, PROSER2-AS1, PROX2, PRPH, PRPH2, PRPS1L1, PRR11, PRR15L, PRR16, PRR19, PRR20B, PRR21, PRR23A, PRR23B, PRR23C, PRR23D2, PRR25, PRR27, PRR29, PRR29-AS1, PRR30, PRR32, PRR34, PRR35, PRR36, PRR5-ARHGAP8, PRR5L, PRR9, PRRG3, PRRT1, PRRT4, PRRX1, PRRX2, PRRX2-AS1, PRSS1, PRSS16, PRSS21, PRSS22, PRSS27, PRSS30P, PRSS33, PRSS35, PRSS36, PRSS37, PRSS38, PRSS3P2, PRSS41, PRSS42, PRSS45, PRSS46, PRSS48, PRSS50, PRSS54, PRSS55, PRSS56, PRSS57, PRSS58, PRTFDC1, PRTG, PRTN3, PRUNE2, PSAPL1, PSCA, PSD, PSD2, PSG1, PSG10P, PSG11, PSG2, PSG3, PSG5, PSG6, PSG7, PSG8, PSKH2, PSMA8, PSMC1, PSORS1C1, PSORS1C2, PSTPIP1, PTCH2, PTCHD1, PTCHD1-AS, PTCHD2, PTCHD3, PTCHD4, PTCRA, PTCSC3, PTENP1-AS, PTF1A, PTGDR, PTGDR2, PTGDS, PTGER1, PTGER2, PTGER4P2-CDK2AP2P2, PTGES, PTGES2-AS1, PTGES3L, PTGIR, PTGIS, PTGS2, PTH, PTH1R, PTH2, PTH2R, PTK7, PTN, PTP4A3, PTPN13, PTPN20, PTPN22, PTPNS, PTPN7, PTPRB, PTPRC, PTPRD-AS1, PTPRD-AS2, PTPRE, PTPRG-AS1, PTPRN, PTPRN2, PTPRO, PTPRQ, PTPRR, PTPRT, PTPRVP, PTPRZ1, PTTG2, PTX3, PTX4, PURG, PVALB, PVRL3-AS1, PWAR1, PWAR4, PWRN1, PWRN2, PWRN3, PWRN4, PXDN, PXDNL, PXT1, PYDC1, PYDC2, PYGO1, PYHIN1, PYY, QRFP, QRFPR, R3HDML, RAB19, RAB25, RAB31, RAB33A, RAB36, RAB39A, RAB39B, RAB3C, RAB3D, RAB40A, RAB40AL, RAB41, RAB42, RAB44, RAB6B, RAB6C, RAB6C-AS1, RAB9B, RAB9BP1, RABL2A, RACGAP1P, RAD21-AS1, RAD21L1, RAD51, RAD51AP1, RAD51AP2, RAD54L, RADIL, RAET1E-AS1, RAET1K, RAG1, RAG2, RAI1-AS1, RALGAPA1P, RALYL, RAMP2, RAMP2-AS1, RAMP3, RANBP17, RANBP3L, RAPGEF3, RAPGEF4-AS1, RASA4, RASA4B, RASA4CP, RASAL1, RASAL2-AS1, RASAL3, RASD2, RASGEF1A, RASGEF1C, RASGRF1, RASGRF2-AS1, RASGRP1, RASGRP2, RASGRP4, RASIP1, RASL10A, RASL10B, RASL11B, RASL12, RASSF10, RASSF9, RAVER2, RAX, RAX2, RBAK-RBAKDN, RBBP8NL, RBFADN, RBFOX1, RBFOX3, RBM11, RBM20, RBM26-AS1, RBM44, RBM46, RBMS3, RBMS3-AS1, RBMS3-AS3, RBMXL2, RBMXL3, RBMY1A3P, RBMY1D, RBMY1E, RBMY1J, RBMY2EP, RBMY2FP, RBMY3AP, RBP2, RBP3, RBPJL, RCOR2, RCSD1, RCVRN, RD3, RD3L, RDH10-AS1, RDH12, RDH8, RDM1, REC114, REG1A, REG1B, REG1P, REG3A, REG3G, REG4, REM1, RENBP, REREP3, RERG, RERG-AS1, RERGL, RESP18, RETN, RETNLB, REXO1L2P, RFPL1, RFPL1S, RFPL2, RFPL3, RFPL4A, RFPL4AL1, RFPL4B, RFTN2, RFX2, RFX4, RFX6, RFX8, RGAG1, RGMA, RGMB-AS1, RGPD1, RGPD2, RGPD3, RGPD4, RGPD4-AS1, RGPD6, RGPD8, RGR, RGS1, RGS10, RGS11, RGS13, RGS17, RGS18, RGS21, RGS22, RGS4, RGS6, RGS7, RGS7BP, RGS8, RGS9BP, RGSL1, RHAG, RHBDL2, RHBDL3, RHBG, RHCE, RHD, RHEBL1, RHO, RHOH, RHOJ, RHOXF1, RHOXF1-AS1, RHOXF1P1, RHOXF2B, RHPN1-AS1, RIBC1, RIBC2, RIC3, RIIAD1, RIMBP2, RIMBP3, RIMBP3C, RIMS1, RIMS2, RIMS4, RIPPLY1, RIPPLY2, RIPPLY3, RIT2, RLBP1, RLN1, RLN2, RLN3, RLTPR, RMDN2-AS1, RMST, RNASE10, RNASE11, RNASE12, RNASE2, RNASE3, RNASE6, RNASE8, RNASE9, RNASEH2B-AS1, RNF112, RNF113B, RNF133, RNF138P1, RNF144A-AS1, RNF148, RNF150, RNF151, RNF157, RNF157-AS1, RNF17, RNF175, RNF182, RNF183, RNF212, RNF212B, RNF217-AS1, RNF219-AS1, RNF222, RNF223, RNF224, RNF39, RNFT2, RNU6-16P, RNU6-48P, RNU6-52P, RNU6-64P, RNU6-66P, RNU6-67P, RNU6-69P, RNU6-71P, RNU6-72P, RNU6-76P, RNU6-78P, RNU6-79P, RNU6-81P, RNY4, ROBO2, ROCK1P1, ROPN1, ROPN1B, ROPN1L, ROPN1L-AS1, ROR1, ROR2, RORA-AS1, RORA-AS2, RORB, RORB-AS1, RP1, RP11-87M18.2, RP1L1, RPE65, RPGRIP1, RPH3A, RPL1OL, RPL13AP17, RPL21, RPL21P28, RPL23AP32, RPL23AP87, RPL29P2, RPL34-AS1, RPL3L, RPL7, RPRM, RPRML, RPS16P5, RPS26P11, RPS2P32, RPS4Y1, RPS4Y2, RPS6KA2-AS1, RPS6KA2-IT1, RPS6KA5, RPS6KA6, RPS7P5, RPSAP52, RPSAP9, RPTN, RRH, RRN3P2, RRS1-AS1, RS1, RSPH1, RSPH10B2, RSPH14, RSPH4A, RSPH6A, RSPH9, RSPO1, RSPO3, RSPO4, RSU1P2, RTBDN, RTCA-AS1, RTEL1-TNFRSF6B, RTKN2, RTL1, RTN1, RTP1, RTP2, RTP5, RUFY4, RUNDC3A, RUNDC3A-AS1, RUNX1-IT1, RUNX1T1, RUNX2, RUNX3, RUSC1-AS1, RUVBL1-AS1, RXFP1, RXFP2, RXFP3, RXFP4, RXRG, RYR1, RYR2, RYR3, S100A12, S100A5, S100A7, S100A7A, S100A7L2, S100A8, S100B, S100G, S100Z, S1PR4, S1PR5, SACS-AS1, SAG, SAGE1, SALL2, SALL3, SALL4, SALRNA1, SALRNA2, SALRNA3, SAMD11, SAMD12-AS1, SAMD13, SAMD14, SAMD15, SAMD3, SAMD7, SAMSN1, SAMSN1-AS1, SAP3OL-AS1, SAPCD2, SASH3, SATB1-AS1, SATB2-AS1, SATL1, SA01, SAXO2, SBF1P1, SBK1, SBK2, SBK3, SBSN, SCAI, SCARA5, SCART1, SCD5, SCEL, SCEL-AS1, SCG2, SCG3, SCGB1A1, SCGB1C1, SCGB1C2, SCGB1D1, SCGB1D2, SCGB1D4, SCGB2A1, SCGB2A2, SCGB3A1, SCGB3A2, SCHLAP1, SCIMP, SCIN, SCML4, SCN10A, SCN11A, SCN1A, SCN2A, SCN2B, SCN3A, SCN3B, SCN4A, SCN4B, SCN5A, SCN7A, SCNN1B, SCNN1G, SCOL-AS1, SCP2D1, SCRG1, SCRN1, SCRT1, SCRT2, SCUBE2, SCUBE3, SDC4P, SDK1, SDK2, SDR16C5, SDR16C6P, SDR9C7, SEBOX, SEC14L3, SEC14L5, SEC14L6, SEC1P, SEC24B-AS1, SEL1L2, SELE, SELL, SELP, SELV, SEMA3A, SEMA3C, SEMA3D, SEMA3E, SEMA3G, SEMA4D, SEMA5B, SEMA6A, SEMA6B, SEMA6D, SEMG1, SEMG2, SENOR, SEPSECS-AS1, SEPT12, SEPT14, SEPT3, SEPT4-AS1, SEPT5-GP1BB, SEPT7P9, SERF1B, SERHL, SERHL2, SERPINA13P, SERPINA9, SERPINB10, SERPINB11, SERPINB12, SERPINB13, SERPINB3, SERPINB5, SERPINE3, SERPINI2, SERTAD4, SERTAD4-AS1, SERTM1, SEZ6, SEZ6L, SEZ6L2, SFMBT2, SFRP1, SFRP2, SFRP5, SFTA1P, SFTA2, SFTA3, SFTPA1, SFTPA2, SFTPB, SFTPC, SGCA, SGCD, SGCG, SGCZ, SGIP1, SGOL1, SGPP2, SGSM1, SH2D1A, SH2D1B, SH2D3C, SH2D4B, SH2D6, SH2D7, SH3BP1, SH3GL1P1, SH3GL1P2, SH3GL2, SH3GL3, SH3PXD2A-AS1, SH3RF3, SH3RF3-AS1, SH3TC2, SHANK1, SHANK2-AS1, SHANK2-A53, SHC3, SHC4, SHCBP1, SHCBP1L, SHD, SHE, SHISA2, SHISA3, SHISA6, SHISA7, SHISA8, SHISA9, SHOX, SHOX2, SHROOM4, SI, SIAH3, SIDT1, SIGLEC1, SIGLEC10, SIGLEC11, SIGLEC12, SIGLEC14, SIGLEC15, SIGLEC16, SIGLEC17P, SIGLEC5, SIGLEC6, SIGLEC7, SIGLEC8, SIGLEC9, SIGLECL1, SIM1, SIM2, SIRPB2, SIRPD, SIRPG, SIRPG-AS1, SIT1, SIX1, SIX2, SIX3, SIX3-AS1, SIX6, SKA1, SKA3, SKIDA1, SKINTL, SKOR1, SKOR2, SLA, SLA2, SLAMF1, SLAMF6, SLAMF8, SLAMF9, SLC10A2, SLC10A4, SLC10A6, SLC11A1, SLC12A1, SLC12A3, SLC12A5, SLC13A1, SLC13A2, SLC13A4, SLC14A1, SLC14A2, SLC14A2-AS1, SLC15A2, SLC15A5, SLC16A11, SLC16A12-AS1, SLC16A14, SLC16A6, SLC16A8, SLC16A9, SLC17A6, SLC17A7, SLC18A1, SLC18A3, SLC1A3, SLC1A6, SLC1A7, SLC22A11, SLC22A12, SLC22A13, SLC22A14, SLC22A16, SLC22A17, SLC22A20, SLC22A24, SLC22A31, SLC22A6, SLC22A8, SLC24A2, SLC24A3, SLC24A4, SLC24A5, SLC25A12, SLC25A2, SLC25A21, SLC25A31, SLC25A35, SLC25A36, SLC25A3P1, SLC25A41, SLC25A48, SLC25A5-AS1, SLC25A51P1, SLC25A52, SLC25A53, SLC26A10, SLC26A3, SLC26A4, SLC26A4-AS1, SLC26A5, SLC26A7, SLC26A8, SLC26A9, SLC27A6, SLC28A2, SLC28A3, SLC2A1-AS1, SLC2A14, SLC2A3, SLC2A4, SLC30A3, SLC30A8, SLC32A1, SLC34A1, SLC34A2, SLC34A3, SLC35D3, SLC35F1, SLC35F3, SLC35F4, SLC35G3, SLC35G4, SLC35G5, SLC35G6, SLC36A2, SLC36A3, SLC37A2, SLC38A11, SLC38A5, SLC38A8, SLC39A12, SLC39A12-AS1, SLC39A2, SLC44A4, SLC45A1, SLC45A4, SLC46A2, SLC47A2, SLC4A1, SLC4A10, SLC4A3, SLC4A5, SLC4A8, SLC4A9, SLC52A1, SLC52A3, SLC5A1, SLC5A12, SLC5A2, SLC5A4, SLC5A5, SLC5A7, SLC5A8, SLC6A1-AS1, SLC6A10P, SLC6A11, SLC6A14, SLC6A15, SLC6A16, SLC6A17, SLC6A18, SLC6A19, SLC6A2, SLC6A3, SLC6A4, SLC6A5, SLC6A7, SLC7A10, SLC7A11-AS1, SLC7A13, SLC7A14, SLC7A3, SLC7A4, SLC7A5P1, SLC7A5P2, SLC7A8, SLC8A1, SLC8A1-AS1, SLC8A2, SLC8A3, SLC9A2, SLC9A3, SLC9A4, SLC9A7P1, SLC9A9, SLC9A9-AS1, SLC9C1, SLC9C2, SLCO1A2, SLCO1B7, SLCO1C1, SLCO2A1, SLCO4A1, SLCO4A1-AS1, SLCO6A1, SLFN11, SLFN12L, SLFN13, SLFN14, SLIT1, SLIT1-AS1, SLIT2-IT1, SLIT3, SLITRK1, SLITRK2, SLITRK4, SLITRK5, SLITRK6, SLMO1, SLN, SLURP1, SLX1A, SLX1A-SULT1A3, SMA4, SMA5, SMAD1-AS1, SMAD1-AS2, SMAD9, SMARCA5-AS1, SMC1B, SMC2-AS1, SMC5-AS1, SMC01, SMCP, SMCR2, SMCR5, SMG1P1, SMG1P2, SMG1P3, SMG1P5, SMG7-AS1, SMIM10, SMIM10L2A, SMIM10L2B, SMIM17, SMIM2, SMIM2-IT1, SMIM21, SMIM22, SMIM23, SMIM24, SMIM5, SMIM9, SMN2, SMOC2, SMPD3, SMR3A, SMR3B, SMTNL1, SMTNL2, SMYD1, SNAI3, SNAP25, SNAP25-AS1, SNAP91, SNCA, SNCA-AS1, SNCAIP, SNOB, SND1-IT1, SNHG22, SNHG24, SNPH, SNRPD2P2, SNTG1, SNTG2, SNTN, SNX20, SNX29P1, SNX31, SNX32, S0D3, SOGA3, SOHLH1, SOHLH2, SORCS1, SORCS3, SORCS3-AS1, SOST, SOSTDC1, SOWAHD, SOX1, SOX10, SOX11, SOX14, SOX15, SOX17, SOX18, SOX2-OT, SOX21, SOX21-AS1, SOX3, SOX30, SOX8, SOX9-AS1, SP140, SP6, SP7, SP8, SP9, SPACA1, SPACA3, SPACA4, SPACA5B, SPACA6P, SPACA6P-AS, SPACA7, SPAG11A, SPAG11B, SPAG17, SPAG6, SPAG8, SPAM1, SPANXA2, SPANXA2-OT1, SPANXB1, SPANXC, SPANXD, SPANXN1, SPANXN2, SPANXN3, SPANXN4, SPANXN5, SPARC, SPARCL1, SPATA1, SPATA12, SPATA13-AS1, SPATA16, SPATA17, SPATA17-AS1, SPATA19, SPATA22, SPATA3, SPATA3-AS1, SPATA31A1, SPATA31A3, SPATA31A6, SPATA31A7, SPATA31C1, SPATA31C2, SPATA31D1, SPATA31D3, SPATA31D4, SPATA31D5P, SPATA31E1, SPATA32, SPATA4, SPATA42, SPATA45, SPATA6, SPATA8, SPATA8-AS1, SPATA9, SPATC1, SPATS1, SPC24, SPC25, SPDYA, SPDYC, SPDYE1, SPDYE2, SPDYE3, SPDYE4, SPDYE5, SPDYE6, SPDYE7P, SPDYE8P, SPEF1, SPEG, SPEM1, SPERT, SPG20-AS1, SPHAR, SPHKAP, SPI1, SPIB, SPIC, SPIN2A, SPINK13, SPINK14, SPINK2, SPINK4, SPINK5, SPINK6, SPINK7, SPINK8, SPINK9, SPINT3, SPINT4, SPN, SPNS3, SP011, SPOCK2, SPOCK3, SPON1, SPPL2C, SPRNP1, SPRR1B, SPRR2B, SPRR2C, SPRR2D, SPRR2E, SPRR2F, SPRR2G, SPRR4, SPRY3, SPRY4-IT1, SPSB4, SPTA1, SPTBN4, SPTSSB, SPTY2D1-AS1, SPZ1, SRCIN1, SRD5A1P1, SRGAP2-AS1, SRGAP2C, SRGAP2D, SRGAP3, SRGAP3-A53, SRL, SRMS, SRPK3, SRRM3, SRRM4, SRRM5, SRY, SSBP3-AS1, SSC5D, SSMEM1, SSPN, SSPO, SSR4P1, SST, SSTR3, SSTR4, SSTR5, SSTR5-AS1, SSX1, SSX2B, SSX3, SSX4B, SSX5, SSX6, SSX7, SSX8, SSX9, ST13P4, ST18, ST3GAL5-AS1, ST6GAL2, ST6GALNAC1, ST6GALNAC3, ST6GALNAC5, ST7-AS1, ST7-A52, ST7-OT3, ST7-OT4, ST8SIA1, ST8SIA2, ST8SIA3, ST8SIA4, ST8SIA5, ST8SIA6, ST8SIA6-AS1, STAB1, STAB2, STAC, STAC2, STAG3, STAG3L1, STAG3L3, STAM-AS1, STAP1, STAR, STARD6, STARD9, STATH, STAU2-AS1, STC1, STEAP1B, STEAP2-AS1, STEAP3-AS1, STEAP4, STH, STIL, STK31, STK32A, STK32B, STK33, STK4-AS1, STKLD1, STMN2, STMN3, STMND1, STOML3, STON1, STON1-GTF2A1L, STOX1, STPG2, STPG2-AS1, STRA6, STRA8, STRC, STX18-AS1, STX18-IT1, STX19, STXBP5-AS1, STXBP5L, STYK1, SUGT1P1, SUGT1P3, SULT1A4, SULT1C3, SULT1C4, SULT2B1, SULT4A1, SULT6B1, SUMO1P1, SUMO4, SUN3, SUNS, SUPT20HL1, SUPT20HL2, SUSD2, SUSD5, SV2A, SV2B, SV2C, SVEP1, SVILP1, SVOP, SVOPL, SYCE2, SYCN, SYCP1, SYCP2, SYCP2L, SYCP3, SYK, SYN1, SYN2, SYN3, SYNDIG1, SYNDIG1L, SYNE1-AS1, SYNGR4, SYNJ2-IT1, SYNPO2, SYNPO2L, SYNPR, SYNPR-AS1, SYP, SYP-AS1, SYT1, SYT10, SYT11, SYT13, SYT14, SYT14P1, SYT15, SYT16, SYT2, SYT3, SYT4, SYT5, SYT6, SYT8, SYTL1, SYTL3, T, TAAR2, TAAR5, TAAR6, TAAR8, TAAR9, TAC4, TACR1, TACR2, TACR3, TAF1L, TAF4B, TAF7L, TAGAP, TAGLN3, TALI, TAL2, TARID, TARM1, TARP, TAS1R1, TAS1R2, TAS1R3, TAS2R1, TAS2R10, TAS2R13, TAS2R16, TAS2R19, TAS2R3, TAS2R30, TAS2R31, TAS2R38, TAS2R39, TAS2R40, TAS2R41, TAS2R42, TAS2R43, TAS2R46, TAS2R50, TAS2R60, TAS2R7, TAS2R8, TAS2R9, TBATA, TBC1D10C, TBC1D21, TBC1D22A-AS1, TBC1D26, TBC1D28, TBC1D29, TBC1D3, TBC1D32, TBC1D3B, TBC1D3C, TBC1D3H, TBC1D3L, TBC1D3P1-DHX40P1, TBC1D3P2, TBC1D3P5, TBL1Y, TBPL2, TBX1, TBX10, TBX18, TBX2, TBX2-AS1, TBX20, TBX21, TBX22, TBX4, TBX5, TBX5-AS1, TBX6, TBXA2R, TBXAS1, TC2N, TCAF2P1, TCAM1P, TCEAL2, TCEAL5, TCEAL6, TCEAL7, TCEB3B, TCEB3CL2, TCERG1L, TCERG1L-AS1, TCF15, TCF21, TCF23, TCF24, TCF4, TCHH, TCHHL1, TCL1A, TCL1B, TCL6, TCN1, TCONS_00029157, TCP10, TCP10L2, TCP11, TCP11X2, TCTE1, TCTE3, TCTEX1D1, TCTEX1D4, TDGF1, TDGF1P3, TDH, TDRD1, TDRD10, TDRD12, TDRD15, TDRD5, TDRD6, TDRD9, TDRG1, TECRL, TECTA, TEDDM1, TEK, TEKT1, TEKT2, TEKT3, TEKT4, TENM2, TENM3, TENM4, TEPP, TERT, TESPA1, TET1, TET2-AS1, TEX11, TEX12, TEX13A, TEX13B, TEX15, TEX19, TEX21P, TEX22, TEX26, TEX26-AS1, TEX28, TEX33, TEX35, TEX36, TEX36-AS1, TEX37, TEX38, TEX40, TEX41, TEX43, TEX9, TFAMP1, TFAP2A, TFAP2A-AS1, TFAP2B, TFAP2C, TFAP2D, TFAP2E, TFCP2L1, TFDP3, TFEC, TFF1, TFF2, TG, TGFA-IT1, TGIF2LX, TGIF2LY, TGM3, TGM4, TGM5, TGM6, TGM7, TH, THBD, THBS2, THBS4, THCAT155, THEG, THEG5, THEGL, THEMIS, THEMIS2, THOC7-AS1, THRA1/BTR, THSD4-AS1, THSD4-AS2, THSD7A, THSD7B, THY1, TIAM2, TICRR, TIE1, TIFAB, TIGD4, TIGIT, TIMD4, TIMP4, TINAG, TISP43, TKTL1, TKTL2, TLDC2, TLL1, TLL2, TLR10, TLR5, TLR7, TLR8, TLR8-AS1, TLX1, TLX1NB, TLX2, TLX3, TM4SF18, TM4SF19, TM4SF19-AS1, TM4SF19-TCTEX1D2, TM4SF20, TM6SF1, TMC1, TMC2, TMC3, TMC3-AS1, TMC5, TMC6, TMC8, TMCO2, TMCO5A, TMCO5B, TMED11P, TMEFF1, TMEFF2, TMEM105, TMEM108, TMEM108-AS1, TMEM114, TMEM125, TMEM130, TMEM132B, TMEM132C, TMEM132D, TMEM132E, TMEM145, TMEM14E, TMEM150B, TMEM151B, TMEM155, TMEM163, TMEM169, TMEM173, TMEM174, TMEM178A, TMEM178B, TMEM179, TMEM190, TMEM191A, TMEM191B, TMEM191C, TMEM196, TMEM200A, TMEM200C, TMEM202, TMEM204, TMEM207, TMEM210, TMEM211, TMEM212, TMEM212-AS1, TMEM213, TMEM215, TMEM221, TMEM225, TMEM229A, TMEM229B, TMEM232, TMEM235, TMEM236, TMEM239, TMEM240, TMEM244, TMEM246-AS1, TMEM247, TMEM252, TMEM254-AS1, TMEM255A, TMEM255B, TMEM257, TMEM26, TMEM26-AS1, TMEM262, TMEM30C, TMEM31, TMEM35, TMEM5-AS1, TMEM51-AS1, TMEM52B, TMEM59L, TMEM63C, TMEM71, TMEM72, TMEM72-AS1, TMEM75, TMEM86A, TMEM88B, TMEM89, TMEM8C, TMEM95, TMIE, TMIGD1, TMIGD2, TMIGD3, TMOD4, TMPO-AS1, TMPRSS11A, TMPRSS11B, TMPRSS11BNL, TMPRSS11D, TMPRSS11E, TMPRSS11F, TMPRSS11GP, TMPRSS12, TMPRSS13, TMPRSS15, TMPRSS4, TMPRSS4-AS1, TMPRSS5, TMPRSS7, TMSB15A, TMSB15B, TMSB4Y, TNF, TNFAIP6, TNFAIP8L2, TNFRSF13B, TNFRSF13C, TNFRSF17, TNFRSF18, TNFRSF4, TNFRSF8, TNFSF12-TNFSF13, TNFSF15, TNFSF4, TNFSF8, TNIP3, TNMD, TNN, TNNC1, TNNC2, TNNI2, TNNI3, TNNI3K, TNNT1, TNNT3, TNP1, TNP2, TNR, TNRC18P1, TNRC6C-AS1, TNS4, TNXA, TNXB, TOB2P1, TOMM2OL, TOP1P2, TOPAZ1, TOR4A, TOX, TOX2, TP53TG3C, TP53TG3D, TP53TG5, TP73, TPBGL, TPD52L3, TPH1, TPH2, TPI1P3, TPO, TPPP2, TPPP3, TPRG1, TPRG1-AS2, TPRX1, TPRXL, TPSAB1, TPSB2, TPSD1, TPTE, TPTE2, TPTE2P1, TPTE2P3, TPTE2P5, TPTE2P6, TPTEP1, TRABD2B, TRAF3IP3, TRAPPC3L, TRAT1, TRDMT1, TRDN, TREM1, TREM2, TREML1, TREML2, TREML4, TRERNA1, TREX2, TRG-AS1, TRH, TRHDE, TRHR, TRIL, TRIM17, TRIM40, TRIM42, TRIM43, TRIM43B, TRIM46, TRIM48, TRIM49, TRIM49B, TRIM49C, TRIM49D2, TRIM50, TRIM51, TRIM51HP, TRIM53AP, TRIM58, TRIM59, TRIM6-TRIM34, TRIM60, TRIM61, TRIM63, TRIM64, TRIM64B, TRIM64C, TRIM67, TRIM71, TRIM72, TRIM74, TRIM77, TRIML1, TRIML2, TRPA1, TRPC1, TRPC2, TRPC3, TRPC4, TRPC5, TRPC50S, TRPC6, TRPC7, TRPM1, TRPM2-AS, TRPM3, TRPM5, TRPM6, TRPV2, TRPV3, TRPV5, TRPV6, TRY2P, TSC22D1-AS1, TSG1, TSGA10IP, TSGA13, TSHB, TSHR, TSHZ3, TSIX, TSKS, TSL, TSNAX-DISC1, TSPAN10, TSPAN11, TSPAN16, TSPAN18, TSPAN19, TSPAN2, TSPAN32, TSPEAR, TSPY1, TSPY2, TSPY26P, TSPY3, TSPY4, TSPY8, TSPYL6, TSSC2, TSSK1B, TSSK2, TTBK1, TTC16, TTC21B-AS1, TTC23L, TTC24, TTC25, TTC29, TTC30B, TTC34, TTC39A-AS1, TTC39C-AS1, TTC41P, TTC6, TTC9B, TTK, TTLL10, TTLL13P, TTLL6, TTLL9, TTN, TTTY10, TTTY11, TTTY12, TTTY13, TTTY14, TTTY15, TTTY16, TTTY17B, TTTY17C, TTTY18, TTTY19, TTTY1B, TTTY20, TTTY21, TTTY22, TTTY23, TTTY2B, TTTY3, TTTY3B, TTTY4, TTTY4B, TTTY5, TTTY6, TTTY6B, TTTY7B, TTTY8, TTTY8B, TTTY9A, TTTY9B, TTYH1, TTYH2, TUB, TUB-AS1, TUBA3C, TUBA3D, TUBA3E, TUBA3FP, TUBAL3, TUBB1, TUBB2B, TUBB4A, TUBB8, TUBBP5, TULP1, TULP2, TUNAR, TUSC3, TUSC5, TUSC7, TVP23A, TWIST1, TWIST2, TXLNB, TXLNGY, TXNDC12-AS1, TXNDC2, TXNDC8, TXNRD3NB, TYR, TYRP1, UAP1L1, UBASH3A, UBE2DNL, UBE2E1-AS1, UBE2NL, UBE2Q2L, UBE2U, UBL4B, UBOX5-AS1, UBQLN3, UBQLNL, UBTFL1, UBXN10, UBXN10-AS1, UCHL1-AS1, UCMA, UCN3, UCP1, UCP2, UCP3, UG0898H09, UGT1A10, UGT1A8, UGT2A1, UGT3A2, UGT8, UHRF1, ULK4P2, ULK4P3, UMOD, UMODL1, UMODL1-AS1, UNC13A, UNC13C, UNC45B, UNC5A, UNC5C, UNC5D, UNC79, UNC80, UNCX, UNQ6494, UOX, UPK1A-AS1, UPK1B, UPK3A, UPK3B, UQCRBP1, URAD, USH1C, USH1G, USH2A, USHBP1, USP12-AS1, USP17L1, USP17L10, USP17L11, USP17L12, USP17L13, USP17L15, USP17L17, USP17L19, USP17L2, USP17L20, USP17L21, USP17L25, USP17L3, USP17L4, USP17L6P, USP17L7, USP17L8, USP17L9P, USP2-AS1, USP26, USP27X-AS1, USP29, USP32P1, USP32P2, USP50, USP51, USP6, USP9Y, UST, UTAT33, UTF1, UTS2, UTS2R, UTY, VAC14-AS1, VANGL2, VASH1, VASH2, VAT1L, VAV1, VAV3, VAV3-AS1, VAX1, VAX2, VCAM1, VCAN, VCX, VCX2, VCX3A, VCX3B, VCY1B, VEGFC, VENTX, VENTXP1, VENTXP7, VEPH1, VGF, VGLL1, VGLL2, VGLL3, VHLL, VILL, VIP, VIPR1, VIPR1-AS1, VIPR2, VIT, VN1R1, VN1R1OP, VN1R2, VN1R4, VN1R5, VPREB1, VPS13A-AS1, VRTN, VSIG1, VSIG2, VSIG4, VSIG8, VSTM1, VSTM2A, VSTM2A-OT1, VSTM2B, VSTM5, VSX1, VSX2, VWA2, VWA3A, VWA3B, VWA5B1, VWA5B2, VWA8-AS1, VWC2, VWC2L, VWC2L-IT1, VWDE, VWF, WARS2-IT1, WASH3P, WASH5P, WASH7P, WASIR2, WBP11P1, WBP2NL, WBSCR17, WBSCR28, WDFY4, WDR11-AS1, WDR17, WDR38, WDR49, WDR62, WDR63, WDR64, WDR66, WDR86, WDR86-AS1, WDR87, WDR88, WDR93, WEE2, WFDC1, WFDC10A, WFDC10B, WFDC11, WFDC12, WFDC13, WFDC21P, WFDC3, WFDC5, WFDC6, WFDC8, WFDC9, WHAMMP2, WI2-237311.2, WIF1, WIPF1, WISP1, WISP2, WISP3, WNK2, WNK3, WNT1, WNT10A, WNT10B, WNT16, WNT2, WNT2B, WNT3A, WNT6, WNT7A, WNT8A, WNT8B, WNT9B, WSCD2, WSPAR, WT1, WT1-AS, WTAPP1, WTH3DI, WTIP, WWC2-AS1, WWC2-AS2, WWTR1-AS1, XACT, XAGE1B, XAGE1E, XAGE2, XAGE3, XAGE5, XCL1, XCL2, XCR1, XG, XGY2, XIRP1, XIRP2, XIRP2-AS1, XKR3, XKR4, XKR5, XKR6, XKR7, XKRX, XKRY, XLOC_007697, XLOC_008559, XLOC_009911, XRCC2, XRCC6P5, XXYLT1-AS1, XXYLT1-AS2, XYLT1, YBX2, YIPF7, YPEL4, YVVHAEP1, YWHAEP7, YY1P2, ZAN, ZAP70, ZAR1, ZAR1L, ZBBX, ZBED2, ZBED3-AS1, ZBP1, ZBTB20-AS1, ZBTB20-AS3, ZBTB20-AS4, ZBTB32, ZBTB46, ZBTB46-AS1, ZBTB7C, ZBTB8B, ZC2HC1B, ZC3H12B, ZCCHC12, ZCCHC13, ZCCHC16, ZCCHC18, ZCCHC5, ZCWPW2, ZDHHC11, ZDHHC15, ZDHHC19, ZDHHC22, ZDHHC8P1, ZEB2, ZEB2-AS1, ZFAT-AS1, ZFHX4-AS1, ZFP2, ZFP28, ZFP3, ZFP42, ZFP57, ZFP82, ZFP91-CNTF, ZFPM2, ZFPM2-AS1, ZFR2, ZFX-AS1, ZFY, ZG16B, ZGRF1, ZIC3, ZIC4, ZIC5, ZIK1, ZIM2, ZIM2-AS1, ZIM3, ZMAT4, ZNF114, ZNF132, ZNF135, ZNF137P, ZNF14, ZNF154, ZNF157, ZNF169, ZNF185, ZNF197-AS1, ZNF204P, ZNF208, ZNF214, ZNF233, ZNF252P-AS1, ZNF257, ZNF273, ZNF280A, ZNF280B, ZNF285, ZNF286B, ZNF287, ZNF30-AS1, ZNF300P1, ZNF311, ZNF32-AS3, ZNF320, ZNF321P, ZNF322, ZNF33BP1, ZNF347, ZNF350-AS1, ZNF365, ZNF366, ZNF385C, ZNF385D, ZNF385D-AS1, ZNF385D-AS2, ZNF391, ZNF396, ZNF404, ZNF415, ZNF418, ZNF43, ZNF430, ZNF431, ZNF439, ZNF454, ZNF462, ZNF469, ZNF471, ZNF474, ZNF479, ZNF486, ZNF488, ZNF491, ZNF492, ZNF501, ZNF503-AS1, ZNF519, ZNF521, ZNF525, ZNF530, ZNF532, ZNF534, ZNF536, ZNF540, ZNF541, ZNF546, ZNF549, ZNF555, ZNF556, ZNF559-ZNF177, ZNF560, ZNF571-AS1, ZNF572, ZNF575, ZNF578, ZNF582, ZNF599, ZNF607, ZNF610, ZNF611, ZNF624, ZNF625, ZNF625-ZNF20, ZNF630, ZNF630-AS1, ZNF645, ZNF648, ZNF649-AS1, ZNF658, ZNF658B, ZNF660, ZNF663P, ZNF664-FAM101A, ZNF665, ZNF667, ZNF670-ZNF695, ZNF676, ZNF677, ZNF679, ZNF681, ZNF683, ZNF695, ZNF70, ZNF701, ZNF702P, ZNF705A, ZNF705B, ZNF705D, ZNF705E, ZNF705G, ZNF709, ZNF711, ZNF713, ZNF714, ZNF716, ZNF724P, ZNF726, ZNF727, ZNF728, ZNF729, ZNF730, ZNF732, ZNF733P, ZNF735, ZNF737, ZNF738, ZNF750, ZNF763, ZNF772, ZNF781, ZNF788, ZNF790-AS1, ZNF793, ZNF793-AS1, ZNF80, ZNF804A, ZNF804B, ZNF806, ZNF812, ZNF813, ZNF814, ZNF815P, ZNF816-ZNF321P, ZNF826P, ZNF827, ZNF829, ZNF831, ZNF833P, ZNF835, ZNF847P, ZNF85, ZNF853, ZNF860, ZNF876P, ZNF878, ZNF879, ZNF883, ZNF888, ZNF890P, ZNF90, ZNF93, ZNF98, ZNF99, ZNRD1-AS1, ZNRF2P1, ZNRF2P2, ZNRF3-AS1, ZNRF4, ZP1, ZP2, ZP4, ZPBP, ZPBP2, ZPLD1, ZRANB2-AS2, ZSCAN1, ZSCAN10, ZSCAN23, ZSCAN4, ZSCAN5B, ZSWIM2, and ZSWIM5 were classified as non-expressed. Group 1 of non-expressed genes was observed to include 5 disease-associated genes.

AACS, AADACP1, AAGAB, MMP, AAR2, AARSD1, AATBC, ABALON, ABCA11P, ABCA12, ABCA5, ABCB10, ABCB6, ABCB7, ABCB8, ABCB9, ABCC6P2, ABCD4, ABCF1, ABCF3, ABCG2, ABHD12, ABHD14A, ABHD14A-ACY1, ABHD16A, ABHD16B, ABT1, ACAA1, ACAD8, ACADL, ACADM, ACADS, ACAP1, ACAT1, ACBD3, ACER3, AC01, ACO2, ACOT1, ACOT2, ACOT6, ACOT9, ACOX1, ACP1, ACP5, ACRBP, ACRO, ACSL6, ACSM1, ACTBL2, ACTG1P20, ACTG2, ACTL10, ACTL6A, ACTR1B, ACTR3B, ACTR3C, ACVR2B, ACVR2B-AS1, ACY1, ACY3, ACYP1, ACYP2, ADAL, ADAM15, ADAM9, ADAMTS13, ADAMTS7, ADAMTSL4, ADAR, ADAT1, ADCK4, ADCY1, ADCY10P1, ADCY5, ADCY7, ADD1, ADGRA3, ADGRD1, ADGRE5, ADGRF3, ADGRF4, ADGRG7, ADHS, ADIPOR1, ADMS, ADNP, ADNP-AS1, ADNP2, ADO, ADORA2A, ADORA2A-AS1, ADPRHL1, ADPRM, ADRA2A, ADRA2C, ADRBK1, AES, AFAP1L2, AFG3L2, AFTPH, AGA, AGAPS, AGAP6, AGAP9, AGBL3, AGBLS-AS1, AGGF1, AGMAT, AGO2, AGO3, AGPAT1, AGPATS, AHCTF1P1, AHCY, AHCYL1, AHNAK, AHNAK2, AHSA2, ADA, AIFM1, AIG1, AIMP1, AIP, AJUBA, AK3, AKAP10, AKAP17A, AKAP3, AKAP6, AKAP8, AKIP1, AKIRIN1, AKR1A1, AKR7A2, AKT2, AKTIP, ALB, ALDH1A2, ALDH1A3, ALDH1L1-AS1, ALDH1L1-AS2, ALDH3B1, ALDH7A1, ALDH9A1, ALG1, ALG10, ALG10B, ALG11, ALG13, ALG1L, ALG2, ALG3, ALG5, ALG8, ALG9, ALKBH1, ALKBH4, ALLC, ALOX12, ALOX12P2, ALOX5, ALX3, ALYREF, AMD1, AMDHD1, AMER1, AMFR, AMH, AMIGO2, AMIGO3, AMPD1, AMZ2P1, ANAPC10, ANAPC13, ANAPC15, ANAPC16, ANAPC2, ANAPC5, ANAPC7, ANGPTL1, ANGPTL2, ANGPTL6, ANGPTL7, ANKAR, ANKH, ANKIB1, ANKLE2, ANKRD12, ANKRD13C, ANKRD18A, ANKRD22, ANKRD23, ANKRD27, ANKRD33, ANKRD35, ANKRD36, ANKRD36BP1, ANKRD36C, ANKRD40, ANKRD46, ANKRD49, ANKRD53, ANKRD61, ANKS3, ANO8, ANP32B, ANP32C, ANTXR1, ANTXR2, ANXA11, ANXA2R, ANXA4, ANXA5, ANXA6, ANXA7, AOC4P, AOX2P, AP1AR, AP1M1, AP1S1, AP1S2, AP2B1, AP2M1, AP2S1, AP3D1, AP3M2, AP3S1, AP4B1-AS1, AP4M1, AP4S1, APBA3, APEH, APEX1, APH1A, API5, APIP, APITD1-CORT, APLF, APLN, APLP1, APLP2, APMAP, APOA1-AS, APOA1BP, APOBEC3B, APOBEC3F, APOBEC3H, APOC4-APOC2, APOL4, APOLD1, APOO, APOOL, APOPT1, APP, APPL1, APTR, APTX, AQP7P1, ARAF, ARAP1, ARAP3, ARC, ARCN1, ARFGAP1, ARFGAP2, ARFGEF3, ARFIP1, ARFIP2, ARFRP1, ARGLU1, ARHGAP1, ARHGAP17, ARHGAP19, ARHGAP20, ARHGAP22, ARHGAP30, ARHGAP5, ARHGEF3, ARHGEF35, ARHGEF39, ARHGEF7, ARID3A, ARID3C, ARID4B, ARIH2, ARIH2OS, ARL1, ARL16, ARL2BP, ARL3, ARL5A, ARL8A, ARL8B, ARMC10, ARMC12, ARMC6, ARMCX1, ARMCX3, ARMCX6, ARNT, ARPC1A, ARPC2, ARPC3, ARPC4, ARPC4-TTLL3, ARPC5, ARPC5L, ARRB1, ARRB2, ARRDC1-AS1, ARSD, ARSE, ARSF, ARSG, ARSH, ARV1, ASAH1, ASB1, ASB14, ASB3, ASB7, ASB8, ASCC2, ASF1B, ASH1L-AS1, ASIC3, ASIP, ASMTL, ASMTL-AS1, ASNA1, ASPRV1, ASPSCR1, ASS1, ASTE1, ATAD1, ATF1, ATF7, ATG101, ATG12, ATG16L1, ATG4A, ATG4B, ATG5, ATL3, ATP11B, ATP1A1-AS1, ATP2A1, ATP5A1, ATP5B, ATP5C1, ATP5F1, ATP5G1, ATP5G2, ATP5J, ATP5J2, ATP5J2-PTCD1, ATP50, ATP5S, ATP5SL, ATP6AP1, ATP6AP1L, ATP6AP2, ATP6V0A1, ATP6V0A2, ATP6V0B, ATP6V0D1, ATP6V0E1, ATP6V0E2, ATP6V1A, ATP6V1C1, ATP6V1D, ATP6V1E2, ATP6V1F, ATP6V1G1, ATP6V1G2-DDX39B, ATP8B2, ATPAF1, ATPAF2, ATRAID, ATXN2L, ATXN3, ATXN7L1, AUP1, AURKAPS1, AVIL, AVL9, AVP, AVPR1A, AXIN1, AXIN2, AZGP1P1, AZI2, AZIN1, AZIN1-AS1, AZIN2, AZU1, B3GALNT1, B3GALNT2, B3GLCT, B3GNT2, B4GALNT3, B4GALT1-AS1, B4GALT2, B4GALT3, B4GALT4, B4GALT7, B9D1, BABAM1, BACE1, BACE1-AS, BACE2, BAG4, BAG6, BAHD1, BAIAP2L2, BAIAP3, BAK1, BANF1, BAP1, BARD1, BATF3, BAX, BAZ2A, BBIP1, BBS12, BBS4, BCAP29, BCAP31, BCAR4, BCCIP, BCDIN3D, BCDIN3D-AS1, BCL10, BCL2L12, BCL2L13, BCL2L2, BCL2L2-PABPN1, BCLAF1, BCO1, BCS1L, BCYRN1, BDH2, BDNF-AS, BEAN1-AS1, BECN1, BEST1, BET1L, BEX1, BEX4, BFAR, BFSP1, BGLAP, BHMT2, BICD2, BID, BIN3, BIRC2, BLOC1S2, BLOC1S5, BLOC1S5-TXNDC5, BLOC1S6, BLVRA, BLVRB, BMI1, BMP8A, BMP8B, BMS1P21, BMS1P4, BMS1P5, BNIP2, BOD1L1, BOLA3, BORA, BPGM, BPHL, BPNT1, BRAP, BRCA1, BRCC3, BRD9, BRE-AS1, BRICD5, BRINP2, BRIX1, BRK1, BROX, BRWD1, BSDC1, BSG, BSPRY, BST2, BTBD1, BTBD10, BTBD16, BTBD6, BTBD7, BTBD8, BTC, BTF3, BTF3L4, BTN2A3P, BUB1B, BUB3, BUD13, BYSL, BZW2, C10orf111, C10orf2, C10orf25, C10orf32, C10orf88, C11orf1, C11orf31, C11orf52, C11orf57, C11orf58, C11orf65, C11orf68, C11orf71, C11orf73, C11orf94, C12orf29, C12orf43, C12orf65, C12orf73, C14orf1, C14orf119, C14orf142, C14orf166, C14orf178, C15orf38-AP3S2, C15orf40, C15orf61, C15orf62, C15orf65, C16orf13, C16orf58, C16orf70, C16orf74, C16orf93, C17orf107, C17orf49, C17orf67, C17orf82, C18orf65, C18orf8, C19orf18, C19orf25, C19orf33, C19orf38, C19orf43, C19orf44, C19orf47, C19orf52, C19orf53, C19orf66, C19orf71, C1D, C1GALT1C1, C1GALT1C1L, C1QB, C1QC, C1QL1, C1QL3, C1QTNF1, C1QTNF1-AS1, C1QTNF4, C1QTNF5, C1QTNF9B-AS1, C1RL, C1orf106, C1orf109, C1orf111, C1orf116, C1orf123, C1orf131, C1orf174, C1orf177, C1orf198, C1orf21, C1orf216, C1orf234, C1orf27, C1orf43, C1orf50, C1orf56, C1orf74, C2-AS1, C20orf196, C21orf33, C21orf58, C22orf23, C22orf29, C22orf39, C2CD4A, C2orf15, C2orf16, C2orf44, C2orf47, C2orf49, C2orf68, C2orf69, C2orf88, C3orf14, C3orf17, C3orf35, C3orf38, C3orf67, C4B, C4B_2, C4orf26, C4orf29, C4orf3, C4orf32, C4orf47, C5AR2, C5orf15, C5orf22, C5orf30, C5orf45, C5orf52, C5orf66, C6orf1, C6orf120, C6orf201, C6orf25, C6orf89, C7orf13, C7orf31, C7orf55, C7orf55-LUC7L2, C7orf60, C7orf61, C7orf73, C8orf33, C8orf44, C8orf48, C8orf76, C8orf89, C9orf114, C9orf116, C9orf117, C9orf152, C9orf153, C9orf156, C9orf24, C9orf40, C9orf43, C9orf57, C9orf69, C9orf78, C9orf9, C9orf92, CA13, CA3, CA3-AS1, CA5BP1, CA9, CAB39, CAB39L, CACFD1, CACNB3, CACTIN, CACUL1, CALCB, CALC00O2, CALM1, CALM3, CALML4, CALML6, CALR, CALU, CAMK1, CAMK1G, CAMK2N1, CAMK2N2, CAMKK2, CAMLG, CAMSAP1, CAMTA1, CAMTA2, CANX, CAP1, CAPN10, CAPN10-AS1, CAPN12, CAPN2, CAPS, CAPZA1, CAPZA2, CARD8-AS1, CARF, CARKD, CARM1, CARNS1, CARS, CARS2, CASC3, CASC4, CASP10, CASP2, CASP7, CASP8AP2, CASS4, CAST, CATIP-AS1, CATSPERG, CBFA2T3, CBFB, CBL, CBLN3, CBS, CBWD1, CBWD2, CBWD6, CBX1, CBX3, CBX8, CCAR1, CCBL2, CCDC103, CCDC110, CCDC115, CCDC121, CCDC13, CCDC130, CCDC134, CCDC150, CCDC152, CCDC157, CCDC158, CCDC159, CCDC163P, CCDC174, CCDC176, CCDC18, CCDC183, CCDC183-AS1, CCDC186, CCDC22, CCDC24, CCDC25, CCDC3, CCDC47, CCDC51, CCDC53, CCDC6, CCDC62, CCDC7, CCDC77, CCDC82, CCDC84, CCDC96, CCDC97, CCHE1, CCK, CCL14, CCL21, CCL3, CCL4, CCL7, CCL8, CCNA2, CCNC, CCND3, CCNDBP1, CCNE2, CCNH, CCNI, CCNK, CCNL1, CCNL2, CCNO, CCNT1, CCNT2, CCNT2-AS1, CCNYL1, CCSER2, CCT4, CCTS, CCT6A, CCT6B, CCT6P1, CCT7, CCT8, CCZ1, CCZ1B, CD151, CD160, CD163, CD164, CD27-AS1, CD274, CD276, CD2AP, CD2BP2, CD300LB, CD302, CD38, CD3D, CD3EAP, CD4, CD47, CD52, CD53, CD59, CD63, CD74, CD79A, CD83, CD9, CD99, CD99L2, CD99P1, CDADC1, CDC123, CDC14A, CDC16, CDC20, CDC25C, CDC27, CDC34, CDC42, CDC42BPB, CDC42BPG, CDC42SE1, CDC45, CDCA2, CDCA4, CDCA5, CDCA8, CDHR3, CDIPT, CDK11A, CDK11B, CDK16, CDK2, CDK3, CDK5R1, CDK7, CDK9, CDKL5, CDKN1B, CDKN1C, CDR2L, CDS2, CDV3, CDYL, CEACAM16, CEACAM6, CEBPB-AS1, CEBPZOS, CECR5, CECR5-AS1, CELF1, CELSR3-AS1, CEMP1, CENPA, CENPBD1P1, CENPJ, CENPK, CENPU, CEP104, CEP135, CEP170, CEP19, CEP44, CEP63, CEP68, CEP70, CEP76, CEP89, CEP95, CEPT1, CERCAM, CERK, CERS2, CERS5, CES1, CES2, CETN2, CETN4P, CFAP126, CFAP221, CFAP36, CFAP53, CFAP57, CFAP69, CFAP70, CFDP1, CFH, CFL1, CFL2, CFLAR, CFLAR-AS1, CGB7, CGREF1, CGRRF1, CHAC2, CHADL, CHCHD4, CHD1L, CHD7, CHD8, CHD9, CHERP, CHFR, CHID1, CHKB, CHM, CHML, CHMP1A, CHMP2A, CHMP2B, CHMP4A, CHMP4B, CHMPS, CHPF, CHPF2, CHPT1, CHRAC1, CHRM3, CHRNA10, CHRNE, CHST1, CHST11, CHST15, CHST7, CHSY1, CHTF8, CHTOP, CHUK, CHURC1, CHURC1-FNTB, CIAO1, CIAPIN1, CIART, CIB1, CIDECP, CINP, CIPC, CIR1, CIRBP-AS1, CISD1, CISD2, CITED1, CKM, CKMT2-AS1, CLASRP, CLCC1, CLCF1, CLCN3, CLCN7, CLDN15, CLDN6, CLDND1, CLEC11A, CLEC3B, CLEC4A, CLIC4, CLINT1, CLIP1-AS1, CLIP4, CLK2P1, CLK3, CLK4, CLMN, CLN3, CLNS, CLN6, CLN8, CLNS1A, CLP1, CLPP, CLPTM1L, CLTB, CLU, CLUH, CLYBL, CMPK1, CMTM1, CMTM4, CMTM6, CMYA5, CNBP, CNEP1R1, CNGA1, CNIH1, CNIH4, CNOT3, CNOT6, CNOT7, CNPPD1, CNPY2, CNPY3, CNPY4, CNST, CNTD2, CNTF, CNTNAP1, COA3, COA5, COASY, COG3, COG4, COG7, COGS, COIL, COL11A2, COL17A1, COL21A1, COL4A3BP, COL6A1, COL6A2, COL9A3, COLCA2, COLEC11, COLGALT1, COMMDS, COMMD7, COMMD8, COMMD9, COMT, COPA, COPB2, COPE, COPG1, COPRS, COPS2, COPS3, COPSS, COPS6, COPS7A, COPS7B, COPSE, COPZ1, COQ2, COQ3, COQ4, COQ6, COQ9, CORO1A, CORO1B, COR07-PAM16, COX10, COX10-AS1, COX11, COX15, COX16, COX18, COX20, COX7A2L, CPD, CPEB2, CPLX1, CPLX4, CPNE1, CPNE3, CPNE7, CPO, CPDX, CPQ, CPSF1, CPSF3L, CPSF6, CPSF7, CPVL, CRABP2, CRAMP1L, CRAT, CRB3, CREB3, CREB3L4, CREBL2, CRELD1, CRELD2, CRHR1-IT1, CRK, CRLF3, CRLS1, CRMP1, CROCCP2, CRSP8P, CRTAP, CRY1, CRYBB2, CRYL1, CRYZ, CRYZL1, CSAD, CSDE1, CSE1L-AS1, CSF2, CSF3, CSGALNACT2, CSK, CSNK1A1, CSNK1A1P1, CSNK1D, CSNK1G2, CSNK1G3, CSNK2A1, CSNK2A2, CSNK2A3, CSNK2B, CSTF3, CTAG2, CTAGE15, CTAGE5, CTAGE8, CTAGE9, CTBP1-AS, CTBS, CTDNEP1, CTDP1, CTDSPL, CTDSPL2, CTNNB1, CTNNBIP1, CTNND1, CTR9, CTRL, CTSA, CTSC, CTSD, CTSO, CTSZ, CTU2, CUL3, CUL4A, CUTA, CWC15, CWF19L1, CXADR, CXCL12, CXCL13, CXCL16, CXCL5, CXCR4, CXXC1, CXorf23, CXorf40B, CXorf56, CYB561, CYB5B, CYB5D2, CYB5R1, CYB5R2, CYB5R4, CYBA, CYBRD1, CYC1, CYCS, CYCSP52, CYFIP1, CYFIP2, CYHR1, CYLD, CYP17A1, CYP20A1, CYP24A1, CYP26A1, CYP26B1, CYP27B1, CYP2A7, CYP2J2, CYP2U1, CYP3A7-CYP3A51P, CYP4A22, CYP7A1, CYSRT1, CYSTM1, CYTH2, DACT3, DAD1, DAGLA, DAGLB, DALRD3, DAP, DAP3, DAPK3, DARS, DARS-AS1, DAXX, DAZAP2, DBF4, DBNDD2, DBNL, DCAF10, DCAF11, DCAF12, DCAF13, DCAF15, DCAF17, DCAF4, DCAF4L1, DCAF5, DCAF7, DCAF8, DCAKD, DCK, DCLRE1C, DCP1A, DCST2, DCTD, DCTN2, DCTN3, DCTN4, DCTN6, DCUN1D3, DCUN1D5, DDA1, DDB1, DDHD2, DDIAS, DDIT4L, DDN, DDOST, DDRGK1, DDTL, DDX1, DDX18, DDX19A, DDX19B, DDX21, DDX24, DDX27, DDX28, DDX31, DDX39A, DDX41, DDX46, DDX47, DDX49, DDX50, DDX51, DDX54, DDX55, DDX6OL, DEAF1, DEAR, DEC1, DECR1, DECR2, DEF8, DEGS1, DEK, DENND1C, DENND6B, DEPDC4, DERA, DERL1, DERL2, DERL3, DESI1, DEXI, DFFA, DFNB59, DGAT1, DGCR11, DGCR2, DGCR5, DGCR6, DGCR8, DGKG, DGKQ, DGKZ, DGUOK, DGUOK-AS1, DHDDS, DHDH, DHPS, DHRS1, DHRS11, DHRS12, DHRS4, DHRS4L1, DHRS7, DHRS7B, DHRSX, DHTKD1, DHX16, DHX34, DHX35, DHX38, DIABLO, DICER1, DICER1-AS1, DIDO01, DIP2A, DIP2A-IT1, DIRAS3, DIS3, DISP2, DKFZP434I0714, DLEU1, DLEU2, DLG3, DLG4, DLST, DLSTP1, DMAP1, DMTF1, DMWD, DNA2, DNAAF3, DNAH11, DNAJA3, DNAJA4, DNAJB11, DNAJB14, DNAJB2, DNAJB5-AS1, DNAJC1, DNAJC10, DNAJC13, DNAJC15, DNAJC18, DNAJC19, DNAJC2, DNAJC21, DNAJC24, DNAJC27, DNAJC3-AS1, DNAJC4, DNAJC5, DNAJC6, DNAJC7, DNAJC8, DNAJC9, DNASE1, DNASE1L3, DNASE2, DND1, DNMBP-AS1, DNMT3B, DNMT3L, DNPEP, DNTTIP1, DOC2GP, DOCK9-AS2, DOK1, DOK4, DOT1L, DPAGT1, DPF2, DPH3, DPH6-AS1, DPH7, DPM1, DPP7, DPP8, DPP9-AS1, DPY19L1P1, DPY19L3, DPY19L4, DPYSL4, DR1, DRAM2, DRC1, DRD3, DRD4, DRG2, DSC3, DSCR3, DSG1, DSG2, DSP, DSTNP2, DTD2, DTL, DTX2P1-UPK3BP1-PMS2P11, DTX3, DUS1L, DUS4L, DUSP13, DUSP2, DUSP22, DUSP28, DUSP3, DUSP9, DVL3, DXO, DYDC2, DYNC1I1, DYNC1I2, DYNC1LI2, DYNLRB1, DYNLT3, DYRK2, DZANK1, E2F1, E2F3, E2F4, E2F7, E4F1, EAPP, EBF4, EBI3, EBLN2, EBLN3, EBNA1BP2, ECHDC1, ECHS1, ECI2, ECM1, ECSIT, EDC3, EDEM2, EEF1A1, EEF1B2, EEF1E1, EEF1E1-BLOC1S5, EEF1G, EEF2, EEF2KMT, EFCAB12, EFCAB13, EFCAB14, EFCAB2, EFEMP2, EFHD2, EFNA3, EFNB2, EFR3A, EGFL7, EGFL8, EGFR-AS1, EGLN2, EGR2, EI24, EID1, EID2, EID3, EIF1AX, EIF1AX-AS1, EIF2A, EIF2AK1, EIF2AK3, EIF2B1, EIF2B2, EIF2D, EIF2S1, EIF2S3, EIF3A, EIF3B, EIF3D, EIF3E, EIF3F, EIF3G, EIF3H, EIF3I, EIF3J, EIF3K, EIF3L, EIF3M, EIF4E, EIF4E2, EIF4E3, EIF4EBP2, EIF4EBP3, EIF4ENIF1, EIF4G2, EIF5A, EIF5A2, EIF5AL1, EIF6, ELAC1, ELAVL1, ELF4, ELK4, ELL3, ELMO2, ELMOD3, ELOVL2-AS1, ELOVL3, ELOVL5, ELOVL7, ELP2, ELP5, EMC10, EMC3, EMC3-AS1, EMC4, EMC6, EMC7, EMC8, EMC9, EMD, EML1, EML2, EML5, EML6, EMP1, ENDOV, ENG, ENKD1, ENO1-AS1, ENO4, ENOPH1, ENOSF1, ENPP7, ENSA, ENTPD1, ENTPD2, ENTPD3-AS1, ENTPD6, ENY2, EOGT, EP400, EPB41L4B, EPDR1, EPHA1, EPHA4, EPHB2, EPHX3, EPM2A, EPN1, EPO, EPRS, EPS15, EPS8L2, EPT1, ERAP1, ERAP2, ERCC1, ERCC8, ERGIC2, ERGIC3, ERH, ERI1, ERI3-IT1, ERICH3, ERICH6-AS1, ERLEC1, ERLIN1, ERLIN2, ERMARD, ERO1A, ERP44, ERVK13-1, ESAM, ESD, ESF1, ESYT1, ESYT2, ETFA, ETNK1, ETS2, ETV2, EVPLL, EXOC3, EX005, EXOC8, EXOSC1, EXOSC7, EXT2, EYA3, EZR, F10, F11R, F8, FAAH, FAAP100, FABP12, FABP4, FADS6, FAF2, FAH, FAHD1, FAHD2A, FAIM, FAM103A1, FAM107B, FAM111B, FAM118B, FAM120A, FAM120AOS, FAM120C, FAM122B, FAM122C, FAM126B, FAM127A, FAM127B, FAM127C, FAM129A, FAM131A, FAM131B, FAM132A, FAM134A, FAM134C, FAM136A, FAM13A-AS1, FAM149A, FAM149B1, FAM150B, FAM151B, FAM155B, FAM160A1, FAM160B1, FAM161A, FAM161B, FAM166B, FAM169B, FAM171B, FAM173B, FAM174B, FAM175B, FAM177A1, FAM179A, FAM183A, FAM189A2, FAM192A, FAM193B, FAM196A, FAM200A, FAM200B, FAM204A, FAM206A, FAM209A, FAM209B, FAM20A, FAM210A, FAM210B, FAM213A, FAM216A, FAM217B, FAM220A, FAM222A-AS1, FAM227B, FAM228B, FAM229B, FAM231D, FAM32A, FAM35A, FAM3A, FAM3C, FAM43B, FAM46C, FAM47E, FAM47E-STBD1, FAM49B, FAM50B, FAM53B, FAM58A, FAM60A, FAM63B, FAM69A, FAM71A, FAM71E1, FAM76B, FAM83A-AS1, FAM83C, FAM83C-AS1, FAM83F, FAM84A, FAM86DP, FAM86FP, FAM86HP, FAM89A, FAM91A1, FAM96A, FAM98C, FANCA, FANCE, FANCG, FANCI, FANCM, FARP2, FAS, FASLG, FASTK, FASTKD3, FBLL1, FBLN7, FBN1, FBP1, FBXL12, FBXL21, FBXL4, FBXL8, FBXO10, FBXO22, FBXO22-AS1, FBXO27, FBXO28, FBXO3, FBXO31, FBXO45, FBXO46, FBXO48, FBXO5, FBXO7, FBXO8, FBXO9, FBXW2, FBXW4, FBXW4P1, FBXW5, FBXW7, FBXW9, FCF1, FCHO2, FCRL6, FCRLA, FDX1, FDX1L, FECH, FEM1A, FEM1B, FEM1C, FEN1, FER1L4, FERMT3, FES, FETUB, FEZ1, FEZ2, FGD5, FGD5-AS1, FGF11, FGF19, FGFBP3, FGFR1, FGFR1OP, FGFR10P2, FGGY, FGR, FH, FHDC1, FHL1, FIBIN, FIBP, FIGNL2, FITM1, FKBP11, FKBP1A, FKBP1B, FKBP3, FKBP7, FKBP8, FKBP9, FLII, FLJ10038, FLJ21408, FLJ22763, FLJ37453, FLOT1, FLOT2, FLRT2, FLVCR1, FLVCR1-AS1, FMO3, FN3KRP, FNDC4, FNTA, FNTB, FOCAD-AS1, FOLR3, FOPNL, FOXA2, FOXC1, FOXE3, FOXJ2, FOXJ3, FOXK1, FOXN3-AS1, FOXP4-AS1, FOXO1, FOXRED1, FPGS, FRMD3, FRMD6-AS1, FRS3, FRZB, FSBP, FSD1L, FSD2, FSIP1, FSTL4, FTH1P3, FTL, FTSJ2, FUBP3, FUCA2, FUNDC2, FUS, FUT1, FUT10, FUT2, FUT6, FUZ, FYB, FYTTD1, FZD1, FZD4, FZD5, FZD7, FZR1, G3BP2, GM, GABARAP, GABARAPL2, GABPB2, GABRR2, GAD1, GAK, GALE, GALNS, GALNT1, GALNT11, GALNT7, GALR2, GAN, GAPDHS, GAR1, GAS2L3, GAS5-AS1, GAS6, GAST, GATA3, GATAD1, GATAD2B, GATC, GATSL2, GBA2, GBAP1, GBP5, GCAT, GCDH, GCM1, GCSAM, GDAP1, GDE1, GDF9, GDI1, GDI2, GDPD3, GDPGP1, GEMIN8, GEMIN8P4, GFM1, GFM2, GFPT2, GGA1, GGACT, GGCT, GGH, GGNBP2, GHDC, GHITM, GHRLOS, GID4, GID8, GIGYF1, GIGYF2, GIN1, GINM1, GINS4, GJA9-MYCBP, GJC1, GJC3, GJD3, GK5, GLB1, GLE1, GLI1, GLI4, GLIDR, GLIS2, GLIS2-AS1, GLMP, GLO1, GLOD4, GLOD5, GLRX2, GLRX3, GLRX5, GLT8D1, GLT8D2, GLTSCR2, GLUD1, GLUD1P3, GLUD1P7, GLYR1, GM2A, GMEB2, GMFB, GMFG, GMPPA, GMPR2, GNA11, GNA13, GNAI2, GNAI3, GNAL, GNAS, GNAZ, GNB2, GNB2L1, GNB5, GNG10, GNG12-AS1, GNG5, GNG8, GNL1, GNL2, GNLY, GNPAT, GNPTG, GNRH1, GNS, GOLGA2, GOLGA2P10, GOLGA3, GOLGA4, GOLGA5, GOLGA7, GOLGA7B, GOLGA8B, GOLM1, GOLT1B, GORASP1, GOSR2, GOT1, GP5, GPAA1, GPANK1, GPATCH2L, GPATCH4, GPATCH8, GPBAR1, GPBP1L1, GPC3, GPC6-AS1, GPLD1, GPN1, GPN2, GPN3, GPR107, GPR108, GPR137, GPR146, GPR160, GPR161, GPR17, GPR183, GPR35, GPR37, GPR37L1, GPR75, GPR75-ASB3, GPR87, GPR89A, GPRASP1, GPRASP2, GPS1, GPS2, GPX3, GPX4, GRB2, GREM1, GREM2, GRHPR, GRINA, GRK4, GRK6, GRM8, GRN, GRPEL2, GRPR, GRSF1, GRTP1-AS1, GS1-124K5.11, GS1-259H13.2, GSDMD, GSE1, GSKIP, GSN, GSPT1, GSS, GSTA5, GSTA7P, GSTCD, GSTK1, GSTM1, GSTO1, GSTZ1, GSX2, GTF2A2, GTF2F1, GTF2H1, GTF2H3, GTF2H4, GTF2H5, GTF2IP20, GTF3A, GTF3C4, GTF3C6, GTPBP1, GTPBP4, GTPBP6, GTSE1, GUCA2A, GUCD1, GUF1, GUSBP1, GYS2, GZMM, H2AFY, H3F3AP4, HMO, HABP4, HACD3, HADH, HADHA, HADHB, HAGH, HAMP, HAPLN3, HAT1, HAUS2, HAUS6, HAVCR2, HBA1, HBA2, HBS1L, HCAR3, HCCS, HCG17, HCG25, HCG26, HCG27, HCG4, HCG4B, HCG9, HCN4, HOST, HDAC10, HDAC11, HDAC3, HDAC6, HDDC3, HDGF, HDHD2, HDLBP, HEBP1, HEBP2, HECTD3, HELB, HELQ, HERC2P10, HERC2P3, HERC2P9, HERC4, HESS, HESX1, HEXA, HEXB, HEY1, HEY2, HFE2, HGFAC, HGH1, HGS, HGSNAT, HHATL, HHLA3, HIATL1, HIATL2, HIBADH, HIBCH, HIC1, HIF1A-AS2, HINFP, HINT3, HIPK1, HIPK1-AS1, HIPK3, HIRA, HIST1H1A, HIST1H1T, HIST1H2AB, HIST1H2AD, HIST1H2AG, HIST1H2AH, HIST1H2A1, HIST1H2AK, HIST1H2AL, HIST1H2AM, HIST1H2BE, HIST1H2BF, HIST1H2BO, HIST1H3A, HIST1H3B, HIST1H3J, HIST1H4B, HIST1H4C, HIST1H4D, HIST2H2AC, HIST2H2BC, HIST2H2BF, HK1, HKR1, HLA-A, HLA-DMB, HLA-DOB, HLA-DPA1, HLA-DPB1, HLA-DPB2, HLA-DRB1, HLA-F-AS1, HLA-H, HLCS, HM13, HM13-AS1, HMBOX1, HMG20B, HMGB1, HMGB3, HMGCL, HMGN1, HMGN2, HMGN2P46, HMGN3-AS1, HMHA1, HMMR, HMP19, HN1L, HNF4A-AS1, HNRNPAO, HNRNPA3, HNRNPC, HNRNPF, HNRNPH1, HNRNPH2, HNRNPK, HNRNPUL1, HNRNPUL2-BSCL2, HOMER2, HOOK1, HORMAD2-AS1, HP1BP3, HPDL, HPN-AS1, HPRT1, HPS1, HPS3, HRASLS2, HS1BP3, HS3ST2, HS3ST6, HSBP1, HSBP1L1, HSCB, HSD11B1L, HSD17B1, HSD17B11, HSD17B12, HSD17B4, HSD17B8, HSDL1, HSF1, HSPA12A, HSPA1L, HSPA2, HSPA7, HSPA9, HSPB11, HSPBP1, HTATIP2, HTATSF1, HTATSF1P2, HTR7P1, HTRA2, HUNK, HUS1, HYKK, HYPK, IAH1, IAPP, IARS, IARS2, IBA57, IBTK, ICAM2, ICAM3, ICAM4, ICAM5, ICE1, ICT1, ID2-AS1, IDE, IDH3B, IDH3G, ID12, ID01, ID02, IDS, IER3IP1, IFF01, IFI44L, IFIT5, IFITM10, IFITM3, IFITM4P, IFNAR1, IFNGR1, IFNLR1, IFT22, IFT43, IFT52, IFT57, IFT74-AS1, IGBP1, IGBP1P1, IGDCC4, IGF2BP3, IGFBPL1, IGFL1, IGHMBP2, IGSF8, IHH, IK, IKBKB, IKZF3, IKZF4, IKZF5, IL1ORB, IL10RB-AS1, IL12A, IL13RA1, IL17C, IL17RC, IL18BP, IL1B, IL1RL2, IL22RA1, IL27, IL3RA, IL6, IL6ST, IL7R, ILF2, ILF3-AS1, ILK, ILKAP, ILVBL, IMMP1L, IMMT, IMP4, IMPA1, IMPA2, IMPACT, IMPAD1, INCA1, INE1, ING5, INMT, INO80B-WBP1, INO80D, INO80E, INPP5D, INPP5E, INPP5F, INPP5K, INSL5, INTS1, INTS10, INTS12, INTS9, INTU, IP6K2,1PMK, IPO5, IPO7, IPO9, IPPK, IQCC, IQCG, IQCH-AS1, IQGAP1, IRAK1, IRF2BP2, IRF4, IRGQ, IRX3, ISG20L2, ISOC1, ISPD, ISY1, ISY1-RAB43, ITCH, ITFG1, ITFG2, ITFG3, ITGA9, ITGA9-AS1, ITGAE, ITGAL, ITGB1BP1, ITGB1BP2, ITGB2, ITGB3BP, ITIH4-AS1, ITLN2, ITM2B, ITSN2, IVD, JAG2, JAGN1, JKAMP, JMJD7, JMJD7-PLA2G4B, JMJD8, JPH1, JRK, JSRP1, JTB, JUN, KANK3, KANSL1-AS1, KARS, KAT8, KATNA1, KATNB1, KBTBD11-OT1, KBTBD2, KBTBD3, KBTBD4, KCCAT198, KCCAT333, KCNC4, KCND1, KCND2, KCNE2, KCNE5, KCNIP2, KCNIP2-AS1, KCNIP3, KCNJ11, KCNJ14, KCNK5, KCNMB2-AS1, KCNMB3, KCP, KCTD18, KCTD2, KCTD21, KCTD21-AS1, KCTD3, KCTD9, KDELC1, KDELR1, KDELR2, KDELR3, KDM1B, KDM3B, KDM4A-AS1, KDM5A, KDM6B, KDSR, KHDRBS1, KHSRP, KIAA0141, KIAA0195, KIAA0226, KIAA0319L, KIAA0355, KIAA0368, KIAA0430, KIAA0753, KIAA0754, KIAA0930, KIAA1191, KIAA1217, KIAA1324, KIAA1429, KIAA1462, KIAA1522, KIAA1524, KIAA1683, KIAA1715, KIAA2013, KIAA2018, KIAA2026, KIDINS220, KIF23, KIF2A, KIF3C, KIF9, KIF9-AS1, KIFAP3, KIFC1, KIN, KIRREL, KITLG, KL, KLB, KLC1, KLC2, KLC3, KLC4, KLF11, KLF13, KLF3, KLHDC3, KLHL17, KLHL18, KLHL20, KLHL31, KLHL4, KLLN, KLRAP1, KLRG1, KMT2A, KMT2B, KMT2E, KPNA1, KPNA4, KPNAS, KPNA6, KRAS, KRBOX1, KRBOX4, KRI1, KRR1, KRT10, KRT17, KRTAP2-3, KRTAP21-2, KRTAPS-8, KRTAP9-7, KRTCAP2, KRTCAP3, KTN1, KTN1-AS1, KXD1, L2HGDH, L3MBTL1, LACTB2, LAG3, LAGE3, LAMA4, LAMA5-AS1, LAMB2P1, LAMP1, LAMP2, LAMP3, LAMTOR1, LAMTOR2, LAMTOR3, LAMTORS, LAMTORS-AS1, LAP3, LAPTM4A, LAPTM4B, LARP1, LARP1B, LARP4, LARP4B, LARS, LAT, LAT2, LBR, LCE3D, LCMT1-AS1, LCN15, LCOR, LDAH, LEFTY1, LEMD2, LENG1, LENG9, LEPROT, LEPROTL1, LETM2, LETMD1, LFNG, LGALS3BP, LGALS8-AS1, LGALS9C, LGMN, LGR5, LHFP, LHFPL3-AS2, LHPP, LIAS, LIFR, LIG1, LIG4, LIMD1, LIMD1-AS1, LIN37, LIN52, LIN9, LINC00106, LINC00115, LINC00159, LINC00189, LINC00235, LINC00240, LINC00242, LINC00260, LINC00294, LINC00311, LINC00324, LINC00365, LINC00476, LINC00528, LINC00571, LINC00574, LINC00589, LINC00595, LINC00597, LINC00605, LINC00607, LINC00623, LINC00628, LINC00659, LINC00662, LINC00667, LINC00672, LINC00674, LINC00685, LINC00702, LINC00847, LINC00852, LINC00856, LINC00869, LINC00881, LINC00883, LINC00886, LINC00909, LINC00910, LINC00920, LINC00960, LINC00963, LINC00987, LINC00997, LINC00998, LINC00999, LINC01006, LINC01011, LINC01057, LINC01061, LINC01063, LINC01094, LINC01126, LINC01127, LINC01138, LINC01140, LINC01164, LINC01184, LINC01186, LINC01229, LINC01237, LINC01239, LINC01269, LINC01278, LINC01358, LINC01420, LINC01426, LINC01428, LINC01430, LINC01431, LINC01433, LINC01435, LINC01447, LINC01465, LINC01481, LINC01510, LINC01512, LINC01515, LINC01545, LINC01558, LINC01559, LINC01560, LINC01569, LINC01589, LINC01599, LINCR-0001, LINCR-0002, LINS, LIPA, LIPT2, LITAF, LIX1L, LLGL1, LLPH, LLPH-AS1, LMAN2, LMAN2L, LMBR1L, LMBRD1, LMF1, LMNA, LNP1, LNX2, LOC100049716, LOC100126784, LOC100128288, LOC100128398, LOC100128494, LOC100128531, LOC100129034, LOC100129046, LOC100129148, LOC100129216, LOC100129461, LOC100129550, LOC100129917, LOC100130075, LOC100130093, LOC100130331, LOC100130357, LOC100130705, LOC100130987, LOC100131655, LOC100132356, LOC100133091, LOC100233156, LOC100270804, LOC100272217, LOC100286922, LOC100287015, LOC100287036, LOC100288123, LOC100288778, LOC100288842, LOC100288846, LOC100289230, LOC100289361, LOC100289511, LOC100289561, LOC100379224, LOC100505530, LOC100505549, LOC100505666, LOC100505938, LOC100505984, LOC100506082, LOC100506100, LOC100506125, LOC100506142, LOC100506178, LOC100506314, LOC100506606, LOC100506639, LOC100506730, LOC100506804, LOC100506985, LOC100507195, LOC100507291, LOC100507424, LOC100507472, LOC100507557, LOC100507634, LOC100507639, LOC100507642, LOC100630923, LOC100652768, LOC100996447, LOC100996579, LOC101060091, LOC101559451, LOC101926933, LOC101927018, LOC101927027, LOC101927056, LOC101927100, LOC101927151, LOC101927178, LOC101927204, LOC101927269, LOC101927322, LOC101927354, LOC101927365, LOC101927418, LOC101927501, LOC101927765, LOC101927780, LOC101927851, LOC101927854, LOC101927972, LOC101928020, LOC101928069, LOC101928323, LOC101928402, LOC101928445, LOC101928525, LOC101928530, LOC101928791, LOC101928794, LOC101928837, LOC101928885, LOC101929066, LOC101929089, LOC101929128, LOC101929229, LOC101929231, LOC101929372, LOC101929448, LOC101929516, LOC101929524, LOC101929541, LOC101929574, LOC101929626, LOC101929633, LOC101929709, LOC101929718, LOC101929767, LOC101929771, LOC101929829, LOC101930071, LOC102031319, LOC102546298, LOC102723354, LOC102723385, LOC102723703, LOC102723729, LOC102723809, LOC102723927, LOC102724009, LOC102724050, LOC102724084, LOC102724094, LOC102724153, LOC102724312, LOC102724484, LOC102724539, LOC102724552, LOC102724589, LOC102724601, LOC102724691, LOC102724784, LOC102724874, LOC102724919, LOC103611081, LOC104968399, LOC105372795, LOC105373383, LOC105377348, LOC105747689, LOC145783, LOC148413, LOC150776, LOC152225, LOC220729, LOC257396, LOC283335, LOC283788, LOC283922, LOC284009, LOC284344, LOC285074, LOC286059, LOC286437, LOC341056, LOC388282, LOC389641, LOC389906, LOC400927, LOC400927-CSNK1E, LOC400958, LOC401127, LOC401320, LOC440434, LOC553103, LOC554206, LOC642361, LOC642846, LOC642852, LOC643072, LOC643355, LOC643387, LOC644172, LOC644762, LOC645513, LOC646214, LOC646471, LOC646626, LOC646903, LOC648987, LOC652276, LOC653653, LOC653712, LOC654342, LOC727751, LOC727896, LOC728024, LOC728323, LOC728613, LOC728739, LOC728743, LOC728752, LOC729218, LOC729603, LOC729739, LOC729867, LOC729970, LOC730183, LOC81691, LOC93622, LONP1, LONP2, LONRF3, LOX, LOXL2, LPAR2, LPAR6, LPGAT1, LPP-AS2, LRFN4, LRIF1, LRIT3, LRP12, LRP5L, LRP8, LRPPRC, LRRC1, LRRC14, LRRC15, LRRC24, LRRC25, LRRC29, LRRC3, LRRC32, LRRC37A3, LRRC37B, LRRC37BP1, LRRC3C, LRRC41, LRRC42, LRRC47, LRRC49, LRRC56, LRRC59, LRRC69, LRRC8C, LRRCC1, LRRFIP1, LRRFIP2, LRRK2, LRTOMT, LRWD1, LSG1, LSM1, LSM11, LSM12, LSM14A, LSM14B, LSM4, LSM5, LSM7, LSP1, LTA, LTA4H, LTB4R, LTB4R2, LTBP3, LTBR, LURAP1L-AS1, LUZP1, LUZP6, LY6G5C, LY6G6D, LY6G6F, LY75-CD302, LYL1, LYPLA1, LYPLA2P2, LYRM1, LYRM2, LYRM5, LYRM7, LYSMD1, MAB2IL2, MAD2L1, MAD2L1BP, MAD2L2, MADD, MAEA, MAFA, MAFG-AS1, MAFIP, MAGED2, MAGEE1, MAGI2-AS3, MAGOH2P, MAGOHB, MAGT1, MAK16, MAN1A2, MAN1B1, MAN2B2, MANBA, MANEA-AS1, MAP1S, MAP2K1, MAP2K2, MAP2K4P1, MAP2K6, MAP3K1, MAP3K13, MAP3K14-AS1, MAP3K7, MAP4K5, MAP6D1, MAP7D2, MAPK1, MAPK13, MAPK3, MAPK4, MAPK8, MAPK8IP1, MAPK8IP2, MAPK9, MAPKAPK2, MAPKAPK3, MAPKAPK5, MAPKBP1, MAPRE2, MARC2, MARCH1, MARCH3, MARCH5, MARCH7, MARK2, MARK3, MARK4, MARS, MARVELD2, MAST4, MATN1-AS1, MATN4, MATR3, MAU2, MAX, MAZ, MB21D1, MBD1, MBD2, MBIP, MBOAT7, MBTPS1, MC1R, MC5R, MCAT, MCEE, MCFD2, MCL1, MCM10, MCM3AP-AS1, MCM4, MCM8, MCOLN1, MCTS1, MCTS2P, MCUR1, MDC1, MDR, MDGA1, MDH1, MDH2, MDM4, MDP1, ME1, MEA1, MEAF6, MECR, MED14OS, MED15, MED16, MED18, MED20, MED22, MED28, MED29, MED31, MED4, MED4-AS1, MED7, MED9, MEF2B, MEF2BNB, MEF2BNB-MEF2B, MEGF8, MESDC2, MEST, METAP1, METAP2, METRNL, METTL10, METTL12, METTL16, METTL17, METTL21A, METTL21B, METTL22, METTL2A, METTL2B, MEX3B, MFAP3, MFF, MFI2-AS1, MFN1, MFN2, MFNG, MFSD1, MFSD11, MFSD12, MFSD8, MFSD9, MGAM2, MGARP, MGAT4B, MGC16142, MGC16275, MGC70870, MGC72080, MGEA5, MGRN1, MGST2, MGST3, MIA, MIA-RAB4B, MIATNB, MICALCL, MICU2, MIDI IP1-AS1, MIEF2, MIEN1, MIER1, MIF-AS1, MIF4GD, MINOS1-NBL1, MINPP1, MIPEP, MIPOL1, MIS18A, MITD1, MKKS, MKLN1-AS, MKNK1, MKRN1, MKX, MLK7-AS1, MLLT1, MLLT10P1, MLLT4, MLST8, MLXIP, MLYCD, MMACHC, MMGT1, MMP1, MMP11, MMP19, MMP24-AS1, MMP25, MMP9, MMS19, MNT, MOAP1, MOB1A, MOB2, MOB3C, MOB4, M0CS2, M0CS3, MOGAT1, MON1B, MORC2, MORC2-AS1, MORN1, MORN2, MOSPD2, MOSPD3, MPC1, MPC1L, MPC2, MPDU1, MPHOSPH8, MPHOSPH9, MPLKIP, MPND, MPP1, MPP3, MPPE1, MPV17, MPV17L2, MPZL1, MPZL3, MRAP, MRE11A, MREG, MRFAP1, MRGBP, MROH2A, MROH8, MRPL1, MRPL10, MRPL13, MRPL18, MRPL19, MRPL21, MRPL24, MRPL28, MRPL30, MRPL32, MRPL34, MRPL35, MRPL37, MRPL38, MRPL39, MRPL4, MRPL40, MRPL42, MRPL42P5, MRPL43, MRPL44, MRPL45, MRPL45P2, MRPL46, MRPL47, MRPL48, MRPL49, MRPL50, MRPL54, MRPL55, MRPL57, MRPS10, MRPS11, MRPS14, MRPS15, MRPS16, MRPS17, MRPS18A, MRPS21, MRPS22, MRPS25, MRPS27, MRPS33, MRPS35, MRPS36, MRPS5, MRPS7, MRPS9, MRS2, MSANTD1, MSANTD2, MSANTD3-TMEFF1, MSANTD4, MSC-AS1, MSH4, MSL1, MSL3, MSMP, MSRB2, MSS51, MT1IP, MT4, MTA1, MTAP, MTBP, MTCH1, MTCH2, MTDH, MTERF4, MTF2, MTFMT, MTFP1, MTFR1, MTFR1L, MTG1, MTG2, MTHFD2, MTHFD2L, MTIF3, MTMR14, MTMR3, MTMR7, MTMR9, MTRF1L, MTRNR2L2, MTX1, MUC13, MUL1, MURC, MUSTN1, MVB12A, MVP, MXD3, MXRA7, MYBL1, MYL12B, MYL6B, MYLIP, MYLK-AS1, MYLPF, MYO10, MYO19, MYO1C, MYO6, MYO9B, MYSM1, MZF1, MZF1-AS1, MZT1, N4BP1, N4BP2L2, N4BP2L2-IT2, N6AMT2, NAA20, NAA25, NAA35, NAA50, NAAA, NAB1, NAB2, NABP2, NACA, NADK, NADK2, NAF1, NAGA, NAGLU, NAGPA, NANOS1, NANP, NANS, NAP1L1, NAP1L4, NAPA, NAPA-AS1, NAPB, NAPG, NAPRT, NARFL, NARS, NAT6, NAT8B, NAT9, NAV2-AS4, NAV3, NBL1, NBPF10, NBPF12, NBPF15, NBPF18P, NBPF25P, NBPF3, NBPF8, NBPF9, NCAPG2, NCAPH, NCBP2, NCCRP1, NCK1-AS1, NCKAP1, NCL, NCLN, NCOA4, NCOR1, NCSTN, NDFIP1, NDFIP2, NDNL2, NDOR1, NDRG2, NDUFA10, NDUFA12, NDUFA4L2, NDUFA5, NDUFA6, NDUFA7, NDUFA9, NDUFAF1, NDUFAF2, NDUFAF3, NDUFAF5, NDUFAF6, NDUFAF7, NDUFB2-AS1, NDUFB4, NDUFB5, NDUFB9, NDUFC1, NDUFS2, NDUFV1, NDUFV2-AS1, NDUFV3, NEB, NECAB2, NECAP2, NEDD8-MDP1, NEFL, NEK11, NEK4, NEK9, NELFA, NELFB, NELFE, NEMF, NEMP2, NETO2, NEURL1B, NEURL2, NEXN-AS1, NFATC2IP, NFE2L2, NFE2L3, NFKBIB, NFKBID, NFKBIL1, NFS1, NFX1, NFXL1, NFYC, NFYC-AS1, NGDN, NGRN, NHEJ1, NHLH2, NHLRC2, NIFK, NIFK-AS1, NINJ1, NIP7, NIPA1, NIPA2, NIPSNAP3A, NISCH, NIT1, NIT2, NKAIN1, NKD1, NKIRAS1, NKIRAS2, NKRF, NKTR, NLN, NMD3, NME1-NME2, NME2, NME4, NMNAT1, NMNAT3, NMRAL1, NMT1, NMT2, NNAT, NNT, NOA1, NOC4L, NODAL, NOM1, NOMO1, NOMO3, NOP58, NOS2, NOTCH2NL, NOTCH3, NOTUM, NOV, NOXO1, NPB, NPBWR1, NPC2, NPEPL1, NPEPPS, NPHP3, NPIPB15, NPIPB3, NPL, NPLOC4, NPPA, NPPA-AS1, NPPC, NPR1, NPR3, NPRL2, NPTN, NPTN-IT1, NPTX1, NPTXR, NQO2, NR1D2, NR1H2, NR1I3, NR2C2, NR2C2AP, NR4A2, NR4A3, NRAP, NRAS, NRBF2, NRCAM, NRD1, NRG4, NRIP1, NRIR, NRN1L, NSA2, NSDHL, NSF, NSFL1C, NSL1, NSMF, NSRP1, NSUN3, NSUN5, NSUN5P1, NSUN5P2, NT5C, NT5C2, NT5C3B, NT5DC1, NT5DC2, NT5M, NTAN1, NTF3, NTPCR, NUAK1, NUB1, NUBP2, NUCB1, NUDCD1, NUDCD2, NUDCD3, NUDT13, NUDT15, NUDT16L1, NUDT16P1, NUDT17, NUDT21, NUDT4P1, NUDT5, NUDT6, NUDT9, NUFIP1, NUP50, NUP54, NUP62, NUP85, NUPL1, NUS1, NUTF2, NUTM2A-AS1, NVL, NXN, NXT2, OARD1, OAZ1, OAZ2, OAZ3, OBFC1, OCEL1, OCIAD1, OCLM, OCLN, ODF2, OFD1, OGFOD1, OGFOD2, OGFOD3, OGFR, OGFR-AS1, OGFRP1, OGT, OIP5, OIT3, OMP, ONECUT2, OOEP, OOSP1, OPLAH, OPN3, OPTN, OR2A7, OR2B6, OR5AU1, ORAI3, ORAOV1, ORC6, ORMDL1, ORMDL2, ORMDL3, 0S9, OSBPL2, OSGEP, OSGIN2, OSR1, OSTC, OSTCP1, OSTF1, OSTM1, OTP, OTUB1, OTUD4, OTUD5, OTUD6B, OTUD7B, OTULIN, OVOL1, OXA1L, OXR1, OXSM, P2RX5, P2RX6P, P2RY6, P3H2, P3H4, P4HA2-AS1, P4HA3, P4HB, P4HTM, PABPC1, PABPC3, PABPC4, PABPN1, PACS2, PADI1, PAFAH1B2, PAFAH2, PAGR1, PAICS, PAIP1, PAK1, PAK2, PAK3, PAK4, PAK6, PALB2, PAM16, PAN3-AS1, PANDAR, PANK1, PANK4, PANO1, PANX1, PAPD4, PAPOLA, PAQR5, PAQR9-AS1, PARD6G, PARD6G-AS1, PARG, PARGP1, PARK2, PARK7, PARL, PARP1, PARP14, PARP15, PARP6, PARPBP, PARS2, PARVA, PARVB, PASK, PATL1, PATL2, PAX8-AS1, PAXBP1, PAXBP1-AS1, PCAT2, PCAT6, PCAT7, PCBD1, PCBD2, PCBP1, PCBP2, PCBP2-OT1, PCCB, PCDH20, PCDH9-A52, PCDHA1, PCDHAC1, PCDHAC2, PCDHGA10, PCDHGB4, PCDHGB5, PCDHGB6, PCED1A, PCED1B, PCGF1, PCGF5, PCGF6, PCID2, PCMT1, PCNA-AS1, PCNP, PCNXL4, PCP2, PCSK4, PCTP, PCYOX1, PCYOX1L, PDAP1, PDCD2, PDCD4-AS1, PDCD6, PDCD6IP, PDCD7, PDCL3, PDCL3P4, PDDC1, PDE1B, PDE2A, PDE4B, PDE4C, PDE4DIP, PDE8A, PDF, PDGFRL, PDHA1, PDHB, PDHX, PDIA3P1, PDLIM3, PDPK1, PDXDC1, PDZD11, PEA15, PEAR1, PECR, PEF1, PEG3, PELI2, PER2, PERM1, PERP, PEX10, PEX11B, PEX11G, PEX13, PEX16, PEX19, PEX2, PEX26, PEX3, PEX5, PEX6, PEX7, PFN2, PGAM1, PGAM2, PGAP3, PGF, PGGT1B, PGLS, PGM3, PGPEP1, PGS1, PHB, PHB2, PHF11, PHF20, PHF5A, PHF7, PHKA1, PHKG1, PHKG2, PHLDA3, PHLDB3, PHOSPHO2, PHOSPHO2-KLHL23, PHYHIP, PHYKPL, PI3, PI4K2B, PI4KAP2, PI4 KB, PIAS1, PIAS3, PIAS4, PIGBOS1, PIGF, PIGG, PIGH, PIGK, PIGL, PIGO, PIGP, PIGQ, PIGT, PIGV, PIGW, PIGX, PIH1D1, PIH1D2, PIK3CD-AS2, PIK3IP1-AS1, PIN1, PIN4P1, PINK1-AS, PINX1, PIP4K2B, PIP4K2C, PIP5KL1, PIPSL, PIR-FIGF, PTSD, PITPNA, PITPNA-AS1, PITPNB, PITRM1-AS1, PIWIL2, PKD1P6, PKD2L1, PLA2G16, PLAU, PLAUR, PLBD1, PLBD1-AS1, PLCD4, PLCE1, PLCL2, PLCXD2, PLD3, PLEKHA1, PLEKHA6, PLEKHA8, PLEKHA8P1, PLEKHF2, PLEKHG3, PLEKHG4, PLEKHM2, PLGLA, PLGRKT, PLK1, PLOD3, PLP2, PLS3-AS1, PLVAP, PLXNB2, PLXNB3, PLXNC1, PM20D2, PMAIP1, PMF1, PMF1-BGLAP, PMM2, PMP22, PMPCA, PMPCB, PMS2CL, PMS2P1, PMS2P2, PMS2P3, PMS2P4, PNISR, PNKD, PNKP, PNLDC1, PNMA6A, PNN, PN01, PNPLA4, PNPLA6, PNPLA8, PNPO, PNPT1, PNRC2, POC1B-GALNT4, PODXL, PODXL2, POF1B, POFUT1, POFUT2, POGLUT1, POGZ, POLB, POLDIP2, POLDIP3, POLE3, POLR1C, POLR1D, POLR2C, POLR2D, POLR2E, POLR2G, POLR2J2, POLR2J3, POLR2K, POLR3A, POLR3C, POLR3D, POLR3GL, POMGNT1, POMP, POMT1, POMT2, PON2, POP4, POP7, POPDC2, POU3F1, POU6F1, POU6F2-AS1, PP7080, PPA1, PPA2, PPAN-P2RY11, PPARD, PPCDC, PPCS, PPDPF, PPFIA3, PPFIA4, PPHLN1, PPIA, PPIB, PPIE, PPIEL, PPIF, PPIG, PPIH, PPIL1, PPIL3, PPIL4, PPIP5K1, PPM1A, PPM1F, PPM1G, PPM1M, PPP1CA, PPP1CB, PPP1R11, PPP1R12A, PPP1R12B, PPP1R12C, PPP1R14C, PPP1R2, PPP1R21, PPP1R26, PPP1R26-AS1, PPP1R37, PPP1R3D, PPP1R3G, PPP1R7, PPP1R9A, PPP2R1A, PPP2R2C, PPP2R2D, PPP2R3B, PPP2R3C, PPP2R4, PPP2R5A, PPP2R5C, PPP3CC, PPP4C, PPP4R1L, PPP4R2, PPP4R4, PPP6C, PPP6R1, PPP6R2, PPT1, PPT2- EGFL8, PQLC1, PQLC3, PRAF2, PROP, PRDX1, PRDX2, PRDX3, PRDX4, PRDX6, PRELID2, PREPL, PRH1, PRICKLE3, PRKAG1, PRKAG2, PRKAG2-AS1, PRKAR1A, PRKCH, PRKCSH, PRKD2, PRKRIP1, PRLHR, PRLR, PRMT2, PRMT3, PRMT6, PRMT7, PROC, PRODH, PRORSD1P, PROSC, PROX1-AS1, PRPF18, PRPF19, PRPF3, PRPF31, PRPF38B, PRPF39, PRPF4, PRPF40A, PRPF4B, PRPS1, PRPS2, PRR13, PRR14, PRR14L, PRR18, PRR26, PRR4, PRR7-AS1, PRRC2B, PRRG1, PRRG2, PRRG4, PRRT2, PRRT3, PRRT3-AS1, PRX, PSAP, PSEN1, PSENEN, PSG9, PSKH1, PSMA1, PSMA2, PSMA3, PSMA3-AS1, PSMA4, PSMA5, PSMA6, PSMA7, PSMB1, PSMB11, PSMB2, PSMB4, PSMB5, PSMB7, PSMB8-AS1, PSMC2, PSMC3, PSMC3IP, PSMC5, PSMD10, PSMD11, PSMD13, PSMD4, PSMD5-AS1, PSMD6, PSMD7, PSMD8, PSMD9, PSMF1, PSMC2, PTAFR, PTBP3, PTCD1, PTCD3, PTCH1, PTDSS1, PTEN, PTENP1, PTER, PTGER3, PTGER4, PTGES2, PTGES3L-AARSD1, PTGR2, PTGS1, PTHLH, PTK6, PTOV1, PTOV1-AS1, PTOV1-AS2, PTP4A2, PTPDC1, PTPN18, PTPRA, PTPRCAP, PTPRU, PTRHD1, PTS, PTTG1IP, PTTG3P, PUF60, PURB, PUSL1, PVR, PVRIG, PVRIG2P, PVRL1, PVRL2, PVRL3, PVRL4, PWP2, PXYLP1, PYCARD-AS1, PYCR2, PYGB, PYGL, PYGM, PYG02, PYROXD1, PYURF, PYY2, QARS, QDPR, QPCTL, QSOX2, R3HCC1, R3HDM4, RAB11A, RAB11B, RAB11B-AS1, RAB11FIP1, RAB11FIP5, RAB12, RAB18, RAB1A, RAB22A, RAB26, RAB27A, RAB29, RAB2A, RAB2B, RAB30, RAB32, RAB33B, RAB34, RAB35, RAB3A, RAB3IL1, RAB40C, RAB4A, RAB4B, RAB4B-EGLN2, RAB5A, RAB5B, RAB6A, RABEP1, RABGEF1, RABGGTB, RABL6, RAC1, RAC2, RAD1, RAD17, RAD18, RAD21, RAD23A, RAD23B, RAD51D, RAD51L3-RFFL, RAD54L2, RAD9B, RAE1, RAET1E, RAET1G, RALA, RALB, RALBP1, RALY-AS1, RAN, RANBP3, RANGAP1, RANGRF, RAP1A, RAP1B, RAP1GAP2, RAP1GDS1, RAP2A, RAP2C, RAP2C-AS1, RARG, RARRES1, RARS, RARS2, RASA2, RASGRF2, RASSF1-AS1, RASSF2, RASSF3, RASSF6, RASSF8, RBAKDN, RBBP4, RBBP7, RBBP8, RBBP9, RBKS, RBL1, RBM12B-AS1, RBM15B, RBM17, RBM22, RBM23, RBM25, RBM26, RBM27, RBM42, RBM4B, RBM5-AS1, RBM6, RBM8A, RBMX2, RBP7, RBPMS-AS1, RBX1, RCAN2, RCAN3AS, RCC2, RCCD1, RCHY1, RCN1, RDH11, RDH13, RDX, REC8, RECK, RECQL, REEP1, REEP2, REEP3, REEP5, RELA, RELL1, RELN, REM2, REN, REPIN1, RER1, REST, RET, REV1, REV3L, REXO1, REXO2, REXO4, RFC1, RFC2, RFC4, RFC5, RFESD, RFFL, RFK, RFPL3S, RFT1, RFX3-AS1, RFX7, RFXAP, RGL2, RGL4, RGS16, RGS20, RGS5, RHBDD1, RHBDD3, RHOBTB2, RHOC, RHOG, RHOQ, RHOT2, RHOV, RICTOR, RILPL2, RIMKLA, RIMS3, RIN2, RINT1, RIPK3, RIPK4, RITA1, RLIM, RMDN1, RMDN2, RMDN3, RMND5A, RNASE13, RNASE7, RNASEH1, RNASEH2C, RNASEK, RNASEK-C17orf49, RNASET2, RND2, RNF103-CHMP3, RNF11, RNF113A, RNF126, RNF126P1, RNF128, RNF13, RNF130, RNF135, RNF138, RNF139, RNF141, RNF145, RNF149, RNF165, RNF166, RNF170, RNF185, RNF186, RNF20, RNF208, RNF216-IT1, RNF216P1, RNF225, RNF25, RNF31, RNF32, RNF40, RNF44, RNF5, RNF5P1, RNF6, RNF7, RNF8, RNH1, RNMT, RNMTL1, RNPEP, RNPEPL1, RNPS1, RNU1-13P, RNU6-19P, RNU6-28P, RNU6-31P, RNU6-34P, RNU6-35P, RNU6-53P, RNU6-57P, RNU6-83P, RNU86, RNY5, ROBO3, ROBO4, ROR1-AS1, ROS1, RP9P, RPA1, RPA2, RPA4, RPAIN, RPAP1, RPARP-AS1, RPE, RPEL1, RPF2, RPGRIP1L, RPIA, RPL10A, RPL12, RPL13AP3, RPL13AP5, RPL13AP6, RPL14, RPL15, RPL17, RPL18, RPL19, RPL19P12, RPL22, RPL23A, RPL23AP53, RPL23AP64, RPL23AP7, RPL23AP82, RPL27, RPL29, RPL3, RPL31P11, RPL32P3, RPL36A, RPL36A-HNRNPH2, RPL4, RPL5, RPL6, RPL7A, RPL7L1, RPL9, RPLP0P2, RPN1, RPN2, RPP30, RPRD1A, RPRD1B, RPS10, RPS10-NUDT3, RPS11, RPS13, RPS15A, RPS15AP10, RPS17, RPS18P9, RPS23, RPS27, RPS27L, RPS3, RPS3A, RPS4X, RPS6, RPS6KA4, RPS6KB2, RPS6KC1, RPSA, RPUSD2, RQCD1, RRAGA, RRM1, RRM2, RRN3, RRN3P3, RRP1, RRP12, RRP7A, RRP8, RSBN1, RSF1, RSG1, RSL1D1, RSPO2, RSU1, RTCB, RTFDC1, RTN3, RTN4, RTTN, RUFY2, RUFY3, RUNDC1, RUNDC3B, RUVBL2, RWDD2A, RWDD3, RWDD4, RXRB, RYK, S100A1, S100A16, S1PR3, SAA2-SAA4, SACM1L, SAE1, SAFB, SAMD8, SAMM50, SAP18, SAP25, SAP30BP, SAP30L, SAPCD1, SAPCD1-AS1, SAR1A, SAR1B, SARAF, SARM1, SARNP, SART1, SART3, SAV1, SBDS, SBF1, SBF2-AS1, SBN01, SBSPON, SCAF1, SCAMP1-AS1, SCAMP3, SCAMP5, SOAP, SCARB2, SCARF2, SCCPDH, SCFD1, SCGB1B2P, SCGB2B2, SCGB2B3P, SCN1B, SCNN1A, SCO1, SCO2, SCOC, SCP2, SCRIB, SCRN2, SCT, SCTR, SCX, SCYL1, SDAD1, SDC2, SDCBP, SDCBP2-AS1, SDCCAG3, SDF4, SDHA, SDHAF2, SDHAF3, SDHAP1, SDHAP3, SDHB, SDHC, SDHD, SDPR, SDR39U1, SEC11A, SEC13, SEC14L1P1, SEC16A, SEC22B, SEC22C, SEC23A, SEC23B, SEC24B, SEC24C, SEC61A1, SEC61A2, SEC62, SEC63, SECISBP2, SECTM1, SELPLG, SELT, SEMA3B, SEMA3B-AS1, SEMA3F, SEMA4A, SEMA6A-AS1, SENP1, SENP3, SENP3-EIF4A1, SENP5, SENP6, SEP15, SEPHS1, SEPT1, SEPT10, SEPT11, SEPT2, SEPT7, SEPT7-AS1, SERBP1, SERINC1, SERINC2, SERINC3, SERINC4, SERPINA2, SERPINB1, SERPINB2, SERPINB4, SERPINB7, SERTAD2, SET, SETD1A, SETD3, SETD4, SETD5, SETD6, SETD7, SETD9, SETSIP, SF3A2, SF3A3, SF3B1, SF3B2, SF3B6, SFSWAP, SFT2D1, SFT2D3, SFTPD, SFXN1, SGCB, SGCE, SGK494, SGMS1-AS1, SGOL1-AS1, SGPL1, SGPP1, SGSH, SGSM3, SGTA, SH2B1, SH2B3, SH2D2A, SH3BGRL, SH3BP4, SH3BP5, SH3BP5-AS1, SH3BP5L, SH3GLB1, SH3GLB2, SH3YL1, SHANK3, SHARPIN, SHBG, SHC1, SHC2, SHISA5, SHKBP1, SHOC2, SIAE, SIAH1, SIGIRR, SIGMAR1, SIK3, SIN3B, SIRPB1, SIRT2, SIRT5, SIRT6, SIX4, SKP1, SLAIN2, SLAMF7, SLBP, SLC12A2, SLC15A4, SLC16A1-AS1, SLC16A10, SLC16A5, SLC17A5, SLC17A8, SLC18A2, SLC1A5, SLC22A18, SLC22A18AS, SLC22A2, SLC22A4, SLC23A3, SLC24A1, SLC25A16, SLC25A17, SLC25A21-AS1, SLC25A23, SLC25A24, SLC25A26, SLC25A3, SLC25A30-AS1, SLC25A34, SLC25A37, SLC25A38, SLC25A39, SLC25A4, SLC25A43, SLC25A44, SLC25A5, SLC25A51, SLC25A6, SLC27A4, SLC29A1, SLC2A5, SLC2A7, SLC30A10, SLC30A2, SLC30A5, SLC30A7, SLC30A9, SLC31A2, SLC35A1, SLC35A2, SLC35A3, SLC35B1, SLC35B2, SLC35B3, SLC3502, SLC35D1, SLC35E1, SLC35E3, SLC35F5, SLC36A4, SLC38A10, SLC38A6, SLC38A7, SLC39A1, SLC39A13, SLC39A3, SLC39A6, SLC39A7, SLC39A8, SLC39A9, SLC41A3, SLC44A1, SLC44A5, SLC45A2, SLC47A1, SLC5A10, SLC5A11, SLC6A13, SLC6A20, SLC7A60S, SLC7A7, SLC7A9, SLC9A1, SLC9A5, SLC9A6, SLC9A7, SLC9A8, SLC9B1, SLC9B2, SLCO3A1, SLCO4C1, SLCO5A1, SLED1, SLFNL1, SLFNL1-AS1, SLIT2, SLITRK3, SLMAP, SLMO2, SLTM, SLU7, SMAD2, SMAD5, SMAD5-AS1, SMAGP, SMAP1, SMARCC2, SMARCD1, SMARCD2, SMDT1, SMG1P7, SMG5, SMG7, SMIM10L1, SMIM13, SMIM14, SMIM15, SMIM18, SMIM19, SMIM2-AS1, SMIM20, SMIM6, SMIM7, SMNDC1, SMPD1, SMPD4, SMPX, SMU1, SMUG1, SMURF1, SMYD2, SMYD5, SNAI1, SNAP29, SNAP47, SNAPC5, SNAPIN, SNF8, SNHG11, SNHG16, SNHG18, SNHG20, SNHG4, SNRNP70, SNRPA1, SNRPB, SNRPC, SNRPD3, SNRPN, SNTA1, SNURF, SNW1, SNX1, SNX12, SNX14, SNX17, SNX19, SNX22, SNX25, SNX3, SNX4, SNX5, SNX6, SNX7, SNX9, SOAT2, SOCS1, SOCS5, SOCS7, SORD, SOWAHA, SOX2, SOX7, SP1, SP110, SP2, SP2-AS1, SP3, SP4, SP5, SPA17, SPAG5-AS1, SPAG7, SPATA2L, SPATA33, SPATA5L1, SPATA6L, SPATC1L, SPCS1, SPCS2, SPCS3, SPDEF, SPDL1, SPECC1L-ADORA2A, SPEF2, SPESP1, SPG21, SPHK2, SPINT2, SPIRE1, SPNS1, SPOCK1, SPOPL, SPPL2A, SPPL3, SPR, SPRED3, SPRN, SPRR2A, SPRYD3, SPRYD4, SPRYD7, SPSB2, SPTB, SPTLC1, SRA1, SRD5A3, SRD5A3-AS1, SREBF1, SREK1, SRFBP1, SRM, SRP14, SRP19, SRP54, SRP68, SRP72, SRP9, SRPK1, SRPR, SRPRB, SRPX, SRR, SRRM1, SRRM2-AS1, SRSF10, SRSF11, SRSF12, SRSF4, SRSF9, SS18L1, SS18L2, SSBP1, SSBP2, SSC4D, SSFA2, SSNA1, SSR1, SSR2, SSR3, SSSCA1, SSSCA1-AS1, SSTR2, SSU72, SSUH2, ST13, ST20, ST20-MTHFS, ST3GAL4-AS1, ST3GAL6, ST3GAL6-AS1, ST6GALNAC6, ST7L, STAG3L2, STAG3L4, STAG3L5P, STAM, STAMBP, STARD13-AS, STARD3, STARD3NL, STARD4-AS1, STARD7-AS1, STAT2, STAU1, STIM2, STK11, STK16, STK17B, STK19, STK24, STK25, STK26, STK38, STK4, STK40, STMN4, STOML2, STOX2, STRA13, STRADB, STRAP, STRIP2, STRN3, STT3A, STT3B, STUB1, STX10, STX12, STX16, STX17-AS1, STX1B, STX2, STXBP2, STXBP6, STYXL1, SUB1, SUCLA2, SUCLG1, SUCLG2, SUCNR1, SUGP1, SUGP2, SULT1A1, SULT1A2, SULT1B1, SULT1C2, SULT1C2P1, SULT1E1, SUMF1, SUMF2, SUMO1, SUMO1P3, SUMO2, SUMO3, SUN1, SUN2, SUPT4H1, SUPT5H, SUPV3L1, SURF1, SURF6, SUSD4, SUV39H2, SWAP70, SWI5, SYAP1, SYCE1, SYCE1L, SYCE3, SYF2, SYMPK, SYNC, SYNE2, SYNGR1, SYNGR3, SYNJ1, SYNJ2BP, SYNJ2BP-COX16, SYNM, SYPL1, SYS1, SYS1-DBNDD2, SYT7, SYVN1, SZT2, TAAR1, TAAR3, TAB2, TAC1, TAC3, TADA3, TAF1, TAF10, TAF12, TAF15, TAF1A, TAF1A-AS1, TAF1C, TAF1D, TAFS, TAFSL, TAF6, TAF7, TAF9, TAGLN2, TALDO1, TANC2, TANGO2, TANK, TAPBP, TAPBPL, TAPT1-AS1, TARBP2, TARDBP, TARS, TARSL2, TAS2R14, TAS2R20, TAS2R4, TAS2R5, TATDN1, TATDN2, TAZ, TBC1D10B, TBC1D13, TBC1D14, TBC1D17, TBC1D22B, TBC1D30, TBCB, TBK1, TBL2, TBL3, TBP, TBR1, TBRG1, TBRG4, TCAF2, TCAIM, TCAP, TCEAL4, TCEANC, TCEANC2, TCEB3, TCEB3-AS1, TCF19, TCF20, TCF25, TCFLS, TCN2, TCP1, TCP11L1, TCTN2, TDG, TDRP, TEC, TECPR1, TECTB, TEF, TEKT4P2, TEKT5, TELO2, TEN1, TEN1-CDK3, TERC, TERF2, TES, TESC-AS1, TESK2, TET3, TEX101, TEX14, TEX261, TEX264, TEX29, TFAM, TFB2M, TFF3, TFIP11, TFPI2, TFPT, TGDS, TGFB2-AS1, TGFB2-OT1, TGFBRAP1, TGIF1, TGIF2, TGOLN2, TH2LCRR, THAP1, THAP4, THAP5, THAP7-AS1, THCAT158, THEM4, THEMS, THG1L, THNSL2, THOC2, THOC3, THOCS, THOC7, THRA, THRB-AS1, THRIL, THTPA, THUMPD1, THUMPD2, THUMPD3, THUMPD3-AS1, TIAF1, TIAL1, TICAM2, TIGD1, TIGD3, TIGD6, TIGD7, TIMM10B, TIMM13, TIMM17A, TIMM17B, TIMM21, TIMM22, TIMM23, TIMM23B, TIMM44, TIMM50, TIMM8A, TIMMDC1, TINCR, TIPARP-AS1, TJAP1, TJP2, TJP3, TKFC, TLE2, TLE6, TLR6, TLR9, TM2D1, TM2D2, TM2D3, TM4SF1-AS1, TM6SF2, TM7SF3, TM9SF1, TM9SF2, TM9SF3, TMA16, TMBIM1, TMBIM4, TMBIM6, TMCC1-AS1, TMCC3, TMCO1, TMED1, TMED10, TMED10P1, TMED2, TMED3, TMED4, TMED5, TMED7, TMED9, TMEM100, TMEM101, TMEM104, TMEM106A, TMEM106B, TMEM106C, TMEM110, TMEM110-MUSTN1, TMEM119, TMEM120A, TMEM123, TMEM126A, TMEM126B, TMEM127, TMEM143, TMEM147, TMEM147-AS1, TMEM14A, TMEM14B, TMEM14C, TMEM150A, TMEM151A, TMEM161A, TMEM161B-AS1, TMEM167A, TMEM17, TMEM179B, TMEM18, TMEM180, TMEM182, TMEM183A, TMEM183B, TMEM184B, TMEM185A, TMEM186, TMEM189-UBE2V1, TMEM19, TMEM192, TMEM198, TMEM199, TMEM203, TMEM205, TMEM206, TMEM208, TMEM209, TMEM214, TMEM216, TMEM218, TMEM219, TMEM220-AS1, TMEM222, TMEM230, TMEM234, TMEM237, TMEM241, TMEM242, TMEM245, TMEM248, TMEM249, TMEM25, TMEM251, TMEM253, TMEM254, TMEM259, TMEM30A, TMEM33, TMEM38B, TMEM39A, TMEM40, TMEM41B, TMEM42, TMEM43, TMEM44-AS1, TMEM45B, TMEM5, TMEM50B, TMEM51, TMEM55A, TMEM55B, TMEM56-RWDD3, TMEM59, TMEM60, TMEM61, TMEM62, TMEM63A, TMEM64, TMEM65, TMEM69, TMEM74, TMEM86B, TMEM87A, TMEM8A, TMEM8B, TMEM9, TMEM92-AS1, TMEM98, TMEM99, TMEM9B, TMEM9B-AS1, TMOD3, TMPPE, TMPRSS3, TMUB1, TMUB2, TMX1, TMX2, TMX2-CTNND1, TMX3, TNFAIP8L2-SCNM1, TNFAIP8L3, TNFRSF11A, TNFRSF12A, TNFRSF6B, TNFRSF9, TNFSF13, TNFSF18, TNFSF9, TNK1, TNKS2-AS1, TNNI1, TNNT2, TNRC18, TNRC6A, TOB1-AS1, TOLLIP, TOLLIP-AS1, TOM1, TOM1L1, TOMM20, TOMM22, TOMM34, TOMM5, TOMM6, TOMM70A, TONSL, TONSL-AS1, TOP1P1, TOP2A, TOP3A, TOP3B, TOPORS, TOPORS-AS1, TOR1A, TOR1AIP2, TOR2A, TOX4, TP53AIP1, TP53I11, TP53RK, TP63, TPD52L1, TPD52L2, TPGS2, TPI1, TPM3, TPMT, TPP1, TPPP, TPRG1L, TPRKB, TPSG1, TPST2, TRA2A, TRA2B, TRABD, TRABD2A, TRADD, TRAF2, TRAF3, TRAF3IP1, TRAF3IP2-AS1, TRAF6, TRAF7, TRAP, TRAM1L1, TRAM2-AS1, TRAP1, TRAPPC1, TRAPPC2L, TRAPPC3, TRAPPC4, TREML5P, TRERF1, TRHDE-AS1, TRIM11, TRIM14, TRIM22, TRIM27, TRIM31-AS1, TRIM32, TRIM35, TRIM36, TRIM37, TRIM39, TRIM39-RPP21, TRIM4, TRIM44, TRIM54, TRIM56, TRIM69, TRIM9, TRIP6, TRIQK, TRMT1, TRMT10C, TRMT2B, TRMT44, TRMT6, TRMT61A, TRMU, TRNAU1AP, TRNT1, TRO, TROAP, TROVE2, TRPC4AP, TRPM2, TRPM7, TRPS1, TRUB2, TSACC, TSC1, TSC2, TSEN34, TSFM, TSG101, TSGA10, TSN, TSNAX, TSNAIP1, TSPAN1, TSPAN17, TSPAN3, TSPAN31, TSPAN33, TSPYL5, TSR2, TSR3, TSSK3, TSSK4, TST, TSTA3, TTC1, TTC14, TTC17, TTC19, TTC21A, TTC28-AS1, TTC3, TTC30A, TTC32, TTC33, TTC36, TTC37, TTC38, TTC4, TTC7A, TTC9C, TTI1, TTL, TTLL2, TTLL3, TUBA1B, TUBAS, TUBB3, TUBD1, TUBG1, TUBGCP2, TUFM, TUFT1, TUSC1, TUSC2, TUT1, TVP23B, TVP23C, TVP23C-CDRT4, TWF1, TXK, TXLNA, TXN2, TXNDC12, TXNDC15, TXNDC16, TXNDC5, TXNL4A, TXNRD2, TYMSOS, TYRO3P, TYROBP, TYW3, TYW5, U2AF1, U2AF1L4, UBA5, UBAC1, UBAC2-AS1, UBALD1, UBB, UBC, UBE2A, UBE2B, UBE2C, UBE2D2, UBE2D3, UBE2E1, UBE2E3, UBE2E4P, UBE2F, UBE2G2, UBE2I, UBE2J2, UBE2L3, UBE2L6, UBE2M, UBE2N, UBE20, UBE2Q1, UBE2Q1-AS1, UBE2Q2, UBE2Q2P1, UBE2Q2P2, UBE2QL1, UBE2V1, UBE2V2, UBE2W, UBE2Z, UBE3A, UBE3B, UBE4A, UBL3, UBL7, UBLCP1, UBP1, UBR4, UBTD2, UBXN1, UBXN4, UBXN6, UBXN7, UBXN8, UCA1, UCHL3, UCK1, UCK2, UCKL1, UCKL1-AS1, UCN2, UEVLD, UFC1, UFD1L, UFM1, UFSP2, UGDH-AS1, UGGT1, UGGT2, UGP2, UGT1A3, UGT1A4, UGT1A5, UGT1A7, UGT1A9, UGT2A2, UGT2B11, UHRF2, ULBP1, ULBP2, ULK2, ULK3, UMPS, UNC119B, UNC45A, UNC50, UNC5B-AS1, UNC93B1, UPF2, UPF3A, UPF3B, UPK1A, UPK2, UPK3BL, UPP2, UQCC1, UQCC2, UQCRC1, UQCRC2, UQCRFS1, URAHP, URGCP-MRPS24, URI1, URM1, UROD, UROS, USE1, USF1, USF2, USO1, USP10, USP12-AS2, USP14, USP15, USP16, USP19, USP3, USP3-AS1, USP31, USP37, USP39, USP4, USP40, USP42, USP44, USP45, USP46-AS1, USP48, UTP14C, UTP23, UTP3, UTP6, UTS2B, UXT, UXT-AS1, VAMP2, VAMP3, VAMPS, VAMP7, VAPA, VAPB, VARS2, VAT1, VBP1, VCPKMT, VDAC2, VDAC3, VEZF1, VEZT, VGLL4, VHL, VIM-AS1, VIMP, VKORC1L1, VLDLR-AS1, VMA21, VMP1, VOPP1, VPRBP, VPS11, VPS16, VPS25, VPS26A, VPS28, VPS29, VPS35, VPS36, VPS37C, VPS39, VPS45, VPS4A, VPS4B, VPS9D1-AS1, VRK3, VSIG10, VSTM2L, VSTM4, VTA1, VTI1B, VTRNA1-1, VTRNA1-2, VTRNA1-3, VTRNA2-1, VWA9, WAC-AS1, WAS, WASF1, WASF2, WASF3, WASH1, WASH2P, WBP11, WBP2, WBP4, WBP5, WBSCR22, WDFY1, WDFY3-AS2, WDHD1, WDPCP, WDR1, WDR11, WDR13, WDR19, WDR3, WDR33, WDR34, WDR41, WDR43, WDR46, WDR53, WDR59, WDR6, WDR61, WDR73, WDR74, WDR76, WDR77, WDR81, WDR82, WDR83, WDR830S, WDR90, WDSUB1, WDTC1, WDYHV1, WEE2-AS1, WFIKKN1, WFIKKN2, WFS1, WHAMMP1, WHAMMP3, WHSC1L1, WIPF2, WIPI1, WNK4, WNT4, WNT5B, WNT7B, WNT9A, WRAP53, WRAP73, WRB, WRNIP1, WSCD1, XAB2, XIAP, XK, XKR9, XPA, XPNPEP3, XPO1, XPO5, XPO6, XPR1, XRCC3, XRN2, XYLB, YARS, YBEY, YBX3, YBX3P1, YES1, YIF1A, YIF1B, YIPF1, YIPF2, YIPF3, YIPF4, YIPF5, YIPF6, YKT6, YME1L1, YOD1, YPEL1, YTHDC1, YWHAB, YWHAE, YWHAH, YWHAQ, YWHAZ, ZACN, ZASP, ZBED1, ZBED4, ZBED5-AS1, ZBED8, ZBTB1, ZBTB10, ZBTB11, ZBTB11-AS1, ZBTB12, ZBTB17, ZBTB18, ZBTB2, ZBTB22, ZBTB25, ZBTB4, ZBTB47, ZBTB48, ZBTB49, ZBTB5, ZBTB6, ZBTB80S, ZC2HC1C, ZC3H11A, ZC3H12D, ZC3H13, ZC3H15, ZC3H18, ZC3H6, ZC3H7A, ZC3H8, ZC3HAV1, ZCCHC14, ZCCHC17, ZCCHC6, ZCCHC7, ZCCHC9, ZCRB1, ZCWPW1, ZDHHC1, ZDHHC12, ZDHHC20, ZDHHC24, ZDHHC3, ZDHHC4, ZDHHC5, ZDHHC6, ZDHHC7, ZDHHC9, ZEB1, ZFAND1, ZFAND6, ZFC3H1, ZFHX2, ZFP14, ZFP37, ZFP41, ZFP64, ZFP92, ZFPL1, ZFR, ZFX, ZFYVE1, ZFYVE19, ZFYVE27, ZG16, ZGPAT, ZHX1, ZHX1-C8orf76, ZIC2, ZKSCAN1, ZKSCAN4, ZKSCAN5, ZKSCAN7, ZMAT2, ZMYM6NB, ZMYND10, ZMYND11, ZNF101, ZNF12, ZNF124, ZNF136, ZNF138, ZNF140, ZNF16, ZNF174, ZNF177, ZNF19, ZNF192P1, ZNF197, ZNF2, ZNF200, ZNF202, ZNF205-AS1, ZNF213, ZNF213-AS1, ZNF215, ZNF219, ZNF22, ZNF221, ZNF223, ZNF225, ZNF226, ZNF227, ZNF229, ZNF230, ZNF234, ZNF24, ZNF250, ZNF251, ZNF256, ZNF26, ZNF264, ZNF266, ZNF268, ZNF271P, ZNF276, ZNF277, ZNF28, ZNF283, ZNF295-AS1, ZNF3, ZNF300, ZNF304, ZNF316, ZNF317, ZNF32-AS1, ZNF32-AS2, ZNF329, ZNF330, ZNF331, ZNF333, ZNF337-AS1, ZNF33B, ZNF341-AS1, ZNF343, ZNF345, ZNF346, ZNF35, ZNF350, ZNF354C, ZNF358, ZNF384, ZNF394, ZNF398, ZNF408, ZNF417, ZNF423, ZNF425, ZNF426, ZNF428, ZNF436-AS1, ZNF442, ZNF443, ZNF446, ZNF461, ZNF467, ZNF470, ZNF483, ZNF487, ZNF496, ZNF497, ZNF500, ZNF502, ZNF507, ZNF510, ZNF511, ZNF513, ZNF517, ZNF518B, ZNF526, ZNF527, ZNF528-AS1, ZNF529, ZNF529-AS1, ZNF543, ZNF547, ZNF548, ZNF551, ZNF554, ZNF557, ZNF562, ZNF564, ZNF565, ZNF566, ZNF568, ZNF57, ZNF570, ZNF571, ZNF573, ZNF576, ZNF577, ZNF584, ZNF585B, ZNF587, ZNF589, ZNF592, ZNF594, ZNF595, ZNF597, ZNF598, ZNF600, ZNF605, ZNF606, ZNF613, ZNF616, ZNF626, ZNF629, ZNF644, ZNF646, ZNF649, ZNF653, ZNF655, ZNF664, ZNF667-AS1, ZNF669, ZNF670, ZNF671, ZNF674, ZNF674-AS1, ZNF678, ZNF682, ZNF687, ZNF696, ZNF699, ZNF7, ZNF706, ZNF71, ZNF717, ZNF718, ZNF720, ZNF721, ZNF736, ZNF740, ZNF749, ZNF75A, ZNF75D, ZNF76, ZNF761, ZNF765, ZNF766, ZNF767P, ZNF768, ZNF773, ZNF774, ZNF775, ZNF776, ZNF782, ZNF783, ZNF790, ZNF791, ZNF792, ZNF799, ZNF8, ZNF805, ZNF808, ZNF816, ZNF818P, ZNF830, ZNF836, ZNF843, ZNF845, ZNF850, ZNF865, ZNF880, ZNF891, ZNF91, ZNFX1, ZNHIT1, ZNHIT3, ZPR1, ZRANB1, ZRANB2, ZRANB2-AS1, ZRANB3, ZSCAN12P1, ZSCAN2, ZSCAN21, ZSCAN22, ZSCAN25, ZSCAN32, ZSCAN5A, ZSWIM7, ZSWIM8-AS1, ZUFSP, ZXDA, and ZYG11A were identified as static. Group 2 of static genes was observed to include 19 disease-associated genes.

The compounds of Table 69 are known to target at least one gene in one of the 63 canonical pathways in Table 68.

TABLE 69
Number of compounds known to target each pathway

| Pathway | # of compounds |
|---|---|
| 5-HT | 2 |
| ABL | 3 |
| Acetylcholine receptor | 5 |
| Adrenergic receptor | 2 |
| ALK | 1 |
| AMPK | 4 |
| Androgen receptor | 2 |
| Aryl hydrocarbon receptor | 3 |
| ATM/ATR | 2 |
| ATP channel | 1 |
| BCL2 | 3 |
| Calcium channel | 1 |
| Calmodulin | 2 |
| Cannabinoid receptor | 3 |
| c-KIT | 1 |
| c-MET | 6 |
| DNA Damage | 2 |
| Dopamine receptor | 2 |
| EGFR | 6 |
| ER | 1 |
| FAK | 1 |
| FGFR | 2 |
| FLT3 | 1 |
| FXR | 2 |
| GABAergic receptor | 2 |
| GR | 3 |
| GSK-3 | 1 |
| Hedgehog/Smoothened | 4 |
| HIF1 | 2 |
| Hippo | 1 |
| Histamine receptor | 2 |
| Hypoxia activated | 2 |
| IGF-1R/InsR | 5 |
| JAK/STAT | 6 |
| LHR-1 | 2 |
| LXR | 2 |
| MAPK | 6 |
| Mineralcorticoid receptor | 2 |
| mTOR | 3 |
| NF-kB | 1 |
| NOTCH | 1 |
| P53 | 7 |
| PAK | 1 |
| PDGFR | 4 |
| PDK-1 | 2 |
| PI3K/AKT | 8 |
| PIM | 2 |
| PKC | 4 |
| Potassium channel | 1 |
| PPAR | 1 |
| Protein Tyrosine Kinase/RTK | 1 |
| Proton pump | 1 |
| RAR | 3 |
| RAS | 1 |
| ROCK | 1 |
| RXR | 1 |
| S6K | 2 |
| Src | 1 |
| TGF-B | 14 |
| TNF-a | 2 |
| VEGFR | 3 |
| Vitamin D Receptor | 2 |
| WNT | 5 |

Thirty-two distinct signaling protein signatures were identified in the hepatocytes. Each of these signatures was analyzed per treatment to determine whether it was correlated with a significant change in gene expression. Significance was defined as FPKM≥1, q-value≤0.05, and log 2 fold change≥1.5. By these parameters, 11349 protein-coding genes were observed to have significantly changed expression in response to at lease one compound. Table 70 provides the number of genes having significantly changed expression for 59 of the compounds in Table 70 used to stimulate hepatocytes.

TABLE 70
Number of genes having significantly altered expression

| | | # of | | | | # of | |
|---|---|---|---|---|---|---|---|
| ID | Compound | genes | Associated pathway (target) | ID | Compound | genes | Associate pathway (target) |
| 116 | TCS 359 | 63 | Protein Tyrosine Kinase/RTK | 147 | GSK621 | 5 | AMPK (AMPK) |
| | | | (FLT3) | | | | |
| 117 | Cobalt chloride | 1275 | HIF1 (HIF1) | 148 | STA-21 | 6 | JAK/STAT (STAT3) |
| 118 | CH223191 | 198 | Aryl hydrocarbon receptor (AhR) | 149 | SMI-4a | 1436 | PIM (Pim1) |
| 119 | Echinomycin | 7161 | Hypoxia activated (HIF) | 150 | AMG337 | 3 | c-MET (c-Met) |
| 120 | PAF-C-16 | 7 | MAPK (MEK) | 151 | Wnt agonist 1 | 138 | WNT (Wnt) |
| 121 | Bexarotene | 267 | RXR(RXR) | 152 | PRI-724 | 88 | WNT (Wnt) |
| 122 | CD 2665 | 1102 | RAR (RAR) | 153 | ABT-263 | 1 | BCL2 (Pan-Bcl-2) |
| 123 | Pifithrin-μ | 5377 | P53 (p53) | 154 | Axitinib | 5 | VEGFR (Pan-VEGFR) |
| 124 | EB1089 | 68 | Vitamin D receptor (VDR) | 155 | Afatinib | 2010 | EGFR (Pan-ErbB) |
| 125 | BMP4 | 2218 | TGF-β (TGF-B) | 156 | Bosutinib | 2184 | Src (Src) |
| 126 | IWP-2 | 15 | WNT (Wnt) | 157 | Dasatinib | 2970 | ABL (Bcr-Abl) |
| 127 | RITA (NSC 652287) | 105 | P53 (p53) | 158 | Mastinib | 151 | c-KIT (c-Kit) |
| 128 | Calcitrol | 1950 | Vitamin D receptor (VDR) | 159 | Crizotinib | 3265 | c-MET (c-Met) |
| 129 | ACEA | 2 | Cannabinoid receptor (CB1) | 160 | PHA-665752 | 1767 | c-MET (c-Met) |
| 130 | Rimonabant | 1392 | Cannabinoid receptor (CB1) | 161 | GSK1904520A | 2 | IGF-1R/lnsR (IGF-1R/lnsR) |
| 131 | Otenabant | 36 | Cannabinoid receptor (CB1) | 162 | GDC-0879 | 274 | MAPK (Raf) |
| 132 | DLPC | 4 | LHR-1 | 163 | LY294002 | 981 | PI3K/AKT (Pan-PI3K) |
| 133 | LHR-1 antagonist | 204 | LHR-1 | 164 | OSU-03012 | 1786 | PDK-1 (PDK-1) |
| 134 | Wnt3a | 1142 | WNT (Frizzled receptor) | 165 | JNJ-38877605 | 64 | c-MET (c-Met) |
| 135 | Activin | 798 | TGF-β (TGF-beta) | 166 | BMS-754807 | 302 | IGF-1R/lnsR (IGF-1R/lnsR) |
| 136 | Nodal | 863 | TGF- β (TGF-beta) | 167 | TGX-221 | 1 | PI3K/AKT (p110b) |
| 137 | Anti-mullerian hormone | 912 | TGF- β (TGF-beta) | 168 | Regorafenib | 10 | VEGFR (Pan-VEGFR) |
| 138 | GDF2 (BMP2) | 2463 | TGF- β (TGF-beta) | 169 | Thalidomide | 3092 | TNF-α (TNF-α) |
| 139 | GDF10 (BMP3b) | 855 | TGF- β (TGF-beta) | 170 | Amuvatinib | 2835 | PDGFR (PDGFRA) |
| 140 | Oxoglaucine | 31 | PI3J/AKT (PI3K/Akt) | 171 | Etomidate | 2 | GABAergic receptor (GABA) |
| 141 | BMS 195614 | 46 | RAR (RAR) | 172 | Gilmepiride | 39 | Potassium channel |
| | | | | | | | (Potassium channel) |
| 142 | LDN193189 | 67 | TGF-β (ALK2/3) | 173 | Omeprazole | 61 | Proton pump |
| 143 | Varenicline tartrate | 7 | Acetylcholine receptor (AchR) | 174 | Tipifarnib | 34 | RAS (Ras) |
| 144 | Histamine | 20 | Histamine receptor (histamine) | 175 | SP600125 | 193 | MAPK (Jnk) |
| 145 | Chloroquine phosphate | 573 | ATM/ATR (ATM/ATR) | | | | |
| 146 | LJI308 | 1 | S6K (RSK1/2/3) | | | | |

Within the genes in Table 70, 87 of 95 liver disease genes in the NH Genetics Home Reference (GHR) database show significantly changed expression in response to at lease one compound. qRT-PCR validation of the results for simvastatin (1), prednisone (16), and rifampicin (19) showed a correlation of 0.6, 0.58, and 0.99 respectively. Table 71 provides the qRT-PCR validation results. (log 2fc=log 2 fold change in gene expression).

TABLE 71
qRT-PCR Validataion

| | | qRT-PCR | Cuffdiff | Cuffdiff | DESeq | DESeq2 |
|---|---|---|---|---|---|---|
| Gene | Cmpd | log2fc | log2fc | q-value | log2fc | q-value |
| ACAT2 | 1 | 1.52 | 1.22 | 4.50E−03 | 1.2 | 3.90E−114 |
| CYP7A1 | 1 | −0.24 | −1.42 | 4.50E−03 | −1.74 | 9.70E−08 |
| HMGCR | 1 | 2.01 | 1.26 | 4.50E−03 | 1.3 | 4.80E−260 |
| PCSK9 | 1 | 2.53 | 1.07 | 4.50E−03 | 0.88 | 4.80E−07 |
| TNFSF11 | 1 | 0.01 | 1.09 | 4.50E−03 | 1.24 | 6.40E−20 |
| AMACR | 16 | −0.35 | −1.4 | 6.10E−04 | −1.24 | 2.90E−39 |
| ARG1 | 16 | 1.53 | 1.48 | 6.10E−04 | 1.59 | 7.80E−73 |
| CA2 | 16 | −0.1 | −1.05 | 6.10E−04 | −0.95 | 5.50E−43 |
| CIDEC | 16 | 1.1 | 1.02 | 6.10E−04 | 1.19 | 6.40E−16 |
| CPS1 | 16 | 0.61 | 2.14 | 6.10E−04 | 2.17 | 4.40E−174 |
| CYP21A2 | 16 | −0.1 | −1.35 | 6.10E−04 | −1.32 | 6.50E−29 |
| CYP3A4 | 16 | 1.3 | 1.98 | 6.10E−04 | 2.08 | 0.00E+00 |
| ENO3 | 16 | −0.03 | −1.24 | 6.10E−04 | −1.03 | 2.50E−33 |
| G6PC | 16 | 0.76 | 1.02 | 6.10E−04 | 1.03 | 2.70E−61 |
| GYS2 | 16 | 0.71 | 2.21 | 6.10E−04 | 2.49 | 5.50E−53 |
| HGD | 16 | 0.5 | 1.23 | 6.10E−04 | 1.31 | 1.20E−235 |
| HOGA1 | 16 | 1.47 | 1 | 6.10E−04 | 1.04 | 1.50E−18 |
| HPGD | 16 | 0.36 | 2.07 | 6.10E−04 | 2.29 | 2.00E−112 |
| NCF2 | 16 | −0.4 | −1.71 | 6.10E−04 | −1.6 | 4.30E−24 |
| RMRP | 16 | −0.33 | −1.43 | 6.10E−04 | −0.78 | 9.40E−01 |
| SLC37A4 | 16 | 0.99 | 1.16 | 6.10E−04 | 1.18 | 5.90E−76 |
| TAT | 16 | 3.26 | 2.37 | 6.10E−04 | 2.48 | 3.90E−187 |
| TDO2 | 16 | 1.41 | 1.05 | 6.10E−04 | 1.21 | 7.90E−96 |
| AGTR1 | 19 | −0.37 | 1.1 | 1.10E−03 | 1.18 | 1.30E−44 |
| ALAS1 | 19 | 0.3 | 1.45 | 1.10E−03 | 1.47 | 5.30E−140 |
| CA12 | 19 | 0.53 | 1.53 | 1.10E−03 | 1.56 | 1.00E−242 |
| CYP2C19 | 19 | −0.37 | 1.75 | 1.10E−03 | 2.3 | 1.40E−74 |
| CYP3A4 | 19 | 1.5 | 3.21 | 1.10E−03 | 3.29 | 0.00E+00 |
| CYP7A1 | 19 | −1.65 | −1.27 | 1.10E−03 | −1.44 | 6.10E−07 |
| ENO3 | 19 | −0.06 | −1.13 | 1.10E−03 | −0.97 | 1.60E−29 |
| POR | 19 | 0.16 | 1.13 | 1.10E−03 | 1.17 | 2.00E−84 |
| SLC51B | 19 | 1.72 | 1.02 | 1.10E−03 | 1.19 | 2.20E−09 |
| SLC6A20 | 19 | −0.17 | 1.03 | 1.10E−03 | 1.06 | 2.20E−07 |
| SLC6A6 | 19 | −0.14 | 1.28 | 1.10E−03 | 1.39 | 5.10E−26 |
| VLDLR | 19 | 0.63 | 1.69 | 1.10E−03 | 1.71 | 7.70E−162 |

In summary, ODSCs were observed to cover about 50% of gene promoters, and 90% correspond to expressed genes in hepatocytes (35% static and 55% dynamic expression). Genes without an ODSC at the promoter agree with previously defined non-liver pathways, such as neurological or olfactory-associated pathways.

### Example 13. Augmentation of the ODSC Definition

The regions identified as ODSCs were further analyzed to determine whether the regions also contained a target motif to augment the definition developed above. One approach to augmenting the definition of an ODSC is to identify the composition of each ODSC identified above ranked by their dynamic response to the compounds in Table 70.

HiChIP was also performed as described in Example 1 to identify characteristics of gene looping that may be used to augment the present definition of an ODSC. Binning was set at either 5Kbp. The factors included in the analysis are shown in Table 72. (SC=occupancy-dependent signaling center; TSS=transcription start site; TP=tether point(s); and IN=insulated neighborhood)

TABLE 72
Analyzed factors

| Factors | SC | TSS | TP | IN |
|---|---|---|---|---|
| SC | SMC1/27ac | SMC1/27ac | SMC1/CTCF | X |
| TSS | . | Pol | SMC1/CTCF | X |
| TA | . | . | X | X |
| IN | . | . | | CTCF/HiC |

“Tether points” represent the boundaries of an IN including the binding site for CTCF. The priority of interactions determined from the different cell lines is shown in Table 73.

TABLE 73
Priority of interactions

| | SC | TSS | TP | IN |
|---|---|---|---|---|
| SC | High | High | Med. | X |
| TSS | . | Low | Med. | X |
| TP | . | . | X | X |
| IN | . | . | . | Low |

Table 74 provides the basic processing and quality control statistics for HiChIP results from hepatocytes and HepG2 cells stimulated by the compounds in Table 68.

TABLE 74
HiChIP Statistics

| | Valid contacts | Inter- | Intra- | | | Estimated Library |
|---|---|---|---|---|---|---|
| Sample | (% of total) | chromosomal | chromosomal | <20kb | >20kb | Complexity |
| HepG2 | 128M (70%) | 20M (11%) | 109M (60%) | 37M (20%) | 71M (39%) | 313M |
| Hepatocyte | 27M (33%) | 6M (7%) | 21M (26%) | 12M (15%) | 9M (11%) | 92M |

The previously observed “nesting” model was confirmed. For example, the combination of CTCF and cohesin bind the “tether points” of an insulated neighborhood spanning 1-3 Mb. Mediator molecules and additional cohesion result in further gene looping of regions 60-1000 Kb, within the insulated neighborhood.

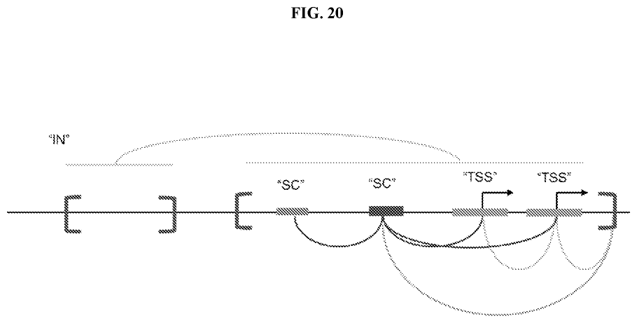

The results above are exemplified by the illustration in FIG. 20 of a single IN. Interactions indicated by the black lines from an ODSC to another ODSC or to a transcription start site (TSS) represent the highest priority of interactions. Interactions indicated by the medium gray lines from a SC (or ODSC) to the TP represent a medium priority interaction, while the interations indicated by the light gray lines from a TSS to another TSS or from a TSS to a TP are a lower priority.

### Example 14. Determining Genome Architecture in Hepatocytes

HI-ChIP was performed as described in Example 1 to decipher genome architecture. In some embodiments, ChIA-PET for SMC1 structural protein is used for the same purpose. These techniques identify portions of the chromatin that interact to form 3D structures, such as insulated neighborhood and gene loops.

Changes in expression of liver disease genes in response to perturbations of GSCs with small molecules as well as information on the 3D architecture of the genome is used to identify relationships (the “signaling code”) among components of GSCs, such as, signaling proteins and transcription factors, as well as genes under their control. This signaling code may be used to predict therapies for inherited diseases.

ENCODE standardized metrics were used to compare the technical quality of a 10 million subsample of the HiChIP data described herein to published data for GM12878 (PMID: 27643841) hepatocytes. The PCR bottlenecking coefficient (PBC1), normalized strand cross-correlation coefficient (NSC), and relative strand cross-correlation coefficient (RSC) were calculated from the initial read data. These values are shown in Table 75.

TABLE 75
Quality control metrics

| Metric | HepG2 | GM12878 |
|---|---|---|
| PBC1 | 0.97 | 0.76 |
| NSC | 1.36 | 1.31 |
| RSC | 4.85 | 2.32 |

The PBC1 for HepG2 cells indicated no bottlenecking, while the PBC1 for the published GM12878 data indicated moderate bottlenecking. The RSC was observed to be significantly higher for the HepG2 cells. Therefore, the HiChIP data for the HepG2 cells was observed to be of a higher quality than previously published data.

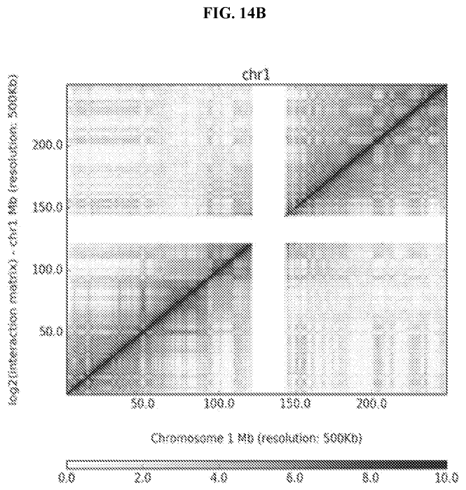

About 77 million paired end tags (PETs) were identified. Of these 77 million, about 55 million were observed to be intrachromosomal, and about 16 million were observed to be interchromosomal. FIG. 14A shows the size distribution of PETs.

Topologically-associated domains (TADs) were clearly observed in FIG. 14B. The boxes indicate insulated neighborhoods. The further the points are off the diagonal dark gray line are more distal of an interaction. The significance of peak-pairs was assessed using Mango and MICC programs. Naïve peaks (700k) in the HepG2 cells were observed to cover more than 80% of public SMC3/CTCF ChIP data from GM12878 (PMID: 27643841) hepatocytes. Similarity was observed between HepG2 data and the published data for GM12878 for PCSK9 gene body, and non-significant linkages were observed to demonstrate deconvolution of the “interaction hairball”.

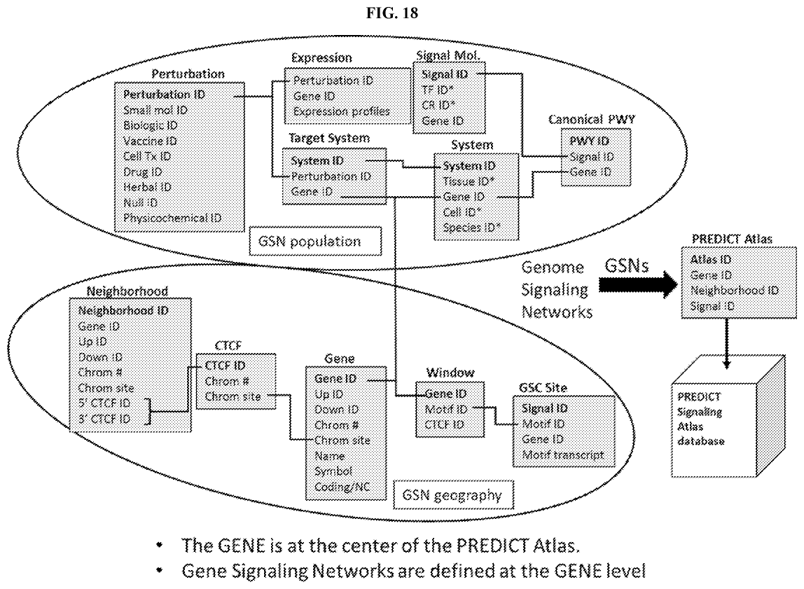

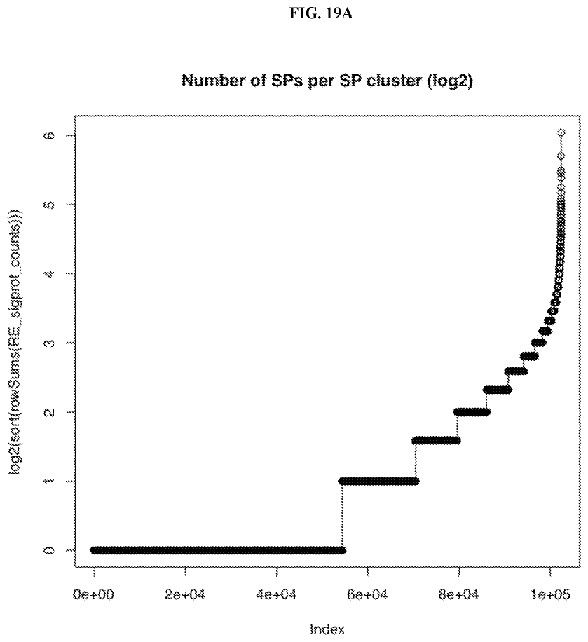

In an effort to build a more rigorous and useful tool for mapping of gene expression, where any particular gene of a primary IN, whether coding or noncoding defines a new signaling pathway, the present inventors have devised an architecture of genome signaling connectivities. Shown in FIG. 18 is one embodiment of the relational aspects of a gene signaling network (GSN).

### Example 15. Conditional Immortalization of Erythroid Cells

The 2D and 3D genomic interrogation of expanding CD34+ and the differentiated erythroblasts allowed identification and understanding of the genomic compositions of genomic signaling centers in the blood. Blood can be readily obtained from diseased patients allowing target validation and compound testing on actual diseased patient cells and models thereof. Deciphering the 2D and 3D maps of normal and diseased blood provides an understanding of the disease biology and allows for screening of new and existing chemical compounds to benefit patients with unmet medical needs.

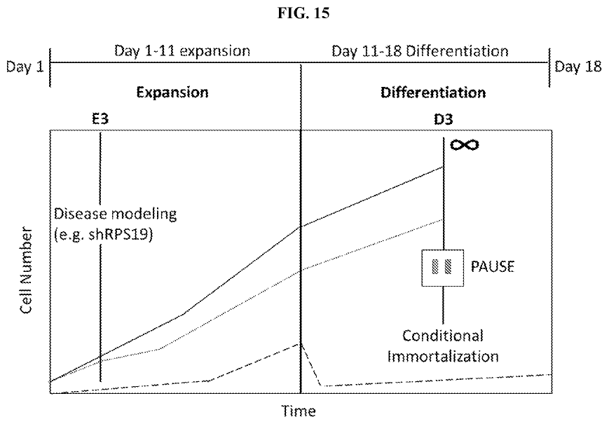

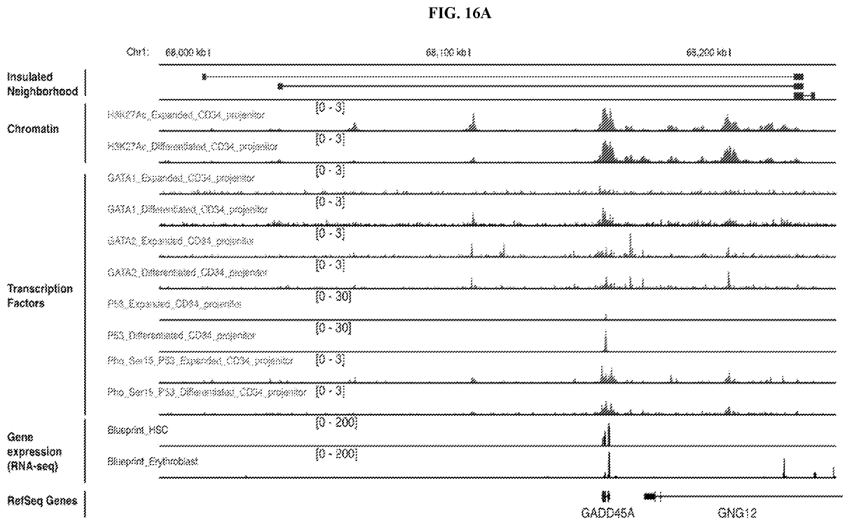

Conditional immortalization allowed for production of a large biomass, while retaining normal cell expression, size, and phenotype. Conditional immortalization was performed as described in Example 1 at day 3 of differentiation. Two samples of cord blood (CB) cells and peripheral blood (PB) CD34⁺ cells containing 1×10⁶ cells each were expanded for 11 days. FIG. 15 provides a summary of the timing of expansion, differentiation, and cell collection. The dash line represents DBA patient cells, and the dotted line represents the conditionally immortalized inducible DBA phenotype model. The solid line represents healthy patient cells.

Antibiotic selection was performed to select virally transformed cells. Cell collection at day 11 showed 100×10⁶ CB cells, and 25×10⁶. Cell collection at day 7 of differentiation showed 400×10⁶ CB cells, and 100×10⁶ peripheral blood cells. Cells conditionally immortalized at day 3 of differentiation were observed to have the capacity to provide a nearly infinite number of cord blood or peripheral blood erythroblast cells. This point in differentiation was chosen as differentiation status was synchronized at the basophilic erythroblast state and cells could be efficiently propagated and cryopreserved. Additionally, this differentiation state is relevant for studying red blood cell diseases.

A. Normal and Conditionally Immortalized Cells Possess Similar Globin Levels

RNA-seq was used to compare the levels of expression of hemoglobin-associated genes among normal and conditionally immortalized cord blood and peripheral blood cells. Table 76 shows the Fragments Per Kilobase of transcript per Million (FPKM) for genes HBB, HBA2, HBA1, HBG1, HBG2, and HBD.

TABLE 76
Levels of expression of hemoglobin-associated genes

| | HBB | HBA2 | HBA1 | HBG2 | HBG1 | HBD |
|---|---|---|---|---|---|---|
| CB_Diff_D 7 | 4838.8 | 5836.9 | 4752.4 | 7140.3 | 2959.8 | 142.5 |
| CB_Diff_D 7 | 8292.7 | 11808.6 | 9886.0 | 17258.2 | 5978.3 | 157.4 |
| PB_Diff_D 7 | 82477.9 | 24625.1 | 12582.8 | 4730.0 | 2874.4 | 3001.4 |
| PB_Diff_D 11 | 78937.4 | 23302.6 | 17447.6 | 10035.3 | 7628.8 | 4114.5 |
| CB_CI_DOX | 405.3 | 4495.7 | 3814.3 | 6710.9 | 2799.0 | 51.8 |
| CB_CI_offDOX | 12472.0 | 39725.3 | 29132.5 | 49890.2 | 23010.6 | 195.2 |
| PB_CI_DOX | 38093.6 | 8791.7 | 5896.0 | 3287.6 | 2028.6 | 1753.2 |
| PB_CI_offDOX | 233336 | 50895.4 | 35108.0 | 12421.1 | 7930.0 | 2188.9 |

These results show no significant differences in the level of expression between normal and conditionally immortalized cells.

### Example 16. Designing Disease Models

Conditionally immortalized erythroblast cells were genetically engineered via CRISPR and shRNA modulation to model DBA. This technique may be applied to model other diseases as desired. This system affords the ability to attain nearly unlimited amounts of primary cell material i.e., the biomass required for 2D and 3D genomic interrogation. As described above, DBA cell viability dramatically decreases during erythroid differentiation (days 11-18) leading to an insufficient number of DBA cells to perform 2D or 3D analysis.

To model the DBA phenotype, hematopoietic progenitor cells and erythroblasts were transduced with short hairpin RNAs (shRNAs) targeting RPS19, one of the key ribosomal proteins identified in DBA patients, and targeting p53 to rescue the DBA phenotype. shRPS19-GFPtrad (gene knock-down) was used to transduce both PB and CB conditionally immortalized erythroid cells with conditional shP53. This allowed expansion of DBA model with protection from overexpression of p53 representing an alternative model of DBA disease. GFPtrad expressing cells were magnetically enriched to avoid the need for additional cell sorting equipment. GFPtrad also allowed for rapid selection of cells when the transgene is toxic to the cells (e.g. shRPS19). The GFP reporter was observed to be inside and outside of the cells to allow multicolor epitope flow cytometry through an anti-GFP antigen presenting cell because only the GFP channel is used.

In certain embodiments, self-delivering RNA (sdRNA) is used in place of a shRNA. The hydrophobic properties of sdRNA allow it to enter cells without need of transfection reagents. Moreover, it is transient allowing more fine-tuned adjustment of knockdown through concentration of the sdRNA. sdRNA can be “washed off” to return RNA to normal levels more quickly than with Dox inducible shRNAs. sdRNA may also be used both in vivo and in vitro.

Alternatively, a Cas9 protein and mRNA sgRNA CRISPR system was used to disrupt a single RPS19 allele in both PB and CB conditionally immortalized erythroid cells with conditional shP53. The efficiency of the CRISPR (NHEJ) was assessed using the GeneArt® Genomic Cleavage Detection Kit (ThermoFisher Scientific) using the standard protocol for the kit.

The DBA phenotype was then conditionally rescued during expansion and differentiation of hematopoietic DBA stem/progenitor cells toward the erythroid lineage. Two different sets of knockdowns were performed, a doxycycline inducible knockdown (mir30 based hairpin design of shRNA) and a constitutive knockdown. Once an effective shRPS19 was identified, cells were treated with compounds to determine whether the disease phenotype was rescued.

DBA cells have 2-4-fold less RPS19 protein than healthy cells. (See, Gazda, H. T. and Sieff, C. A. (2006), British Journal of Haematology, 135: 149-157. doi:10.1111/j.1365-2141.2006.06268.x, which is hereby incorporated by reference in its entirety). shRNA mediated knockdown of RPS19 was compared in conditionally immortalized cord blood erythroblast and normal cells. shRNAs were identified that resulted in 50-70% expression in the conditionally immortalized erythroblast cells, then in normal cells. Alternatively, overexpression of RPS19 was used in DBA patient cells to rescue the DBA phenotype.

During days 1-11 of hematopoietic progenitors (CD34+) cell expansion, the number of DBA patient cells are significantly less than the number of normal patient cells as illustrated in FIG. 15. In fact, the number of DBA patient cells is about a third of the number of normal patient cells by day 11. Rescuing the DBA phenotype by inducible overexpression of RPS, RPL, or GATA1 in conditionally immortalized cells resulted in an increase in cell numbers during expansion and during differentiation. Inducible shRPS19 allowed differentiation of DBA-positive model cells to produce the sufficient number of cells for 2D/3D analysis over the 18 days of expansion and differentiation. The conditionally immortal DBA model may be paused indefinitely to produce from 10⁹ to a nearly infinite number of cells, while the inducible DBA model produced about 10⁶ to 10⁷. Both models result in a greater number of cells than from the primary DBA cells (about 10⁴ to 10⁵).

Examples below describe an immortalized cord-blood erythroblast cell line to produce a large biomass for analysis but that also retains normal cell expression, size, and phenotype.

A DBA model cell system is set up through shRNA-mediated knockdown of RPS19 or CRISPR removal of RPS19 in the conditionally immortalized cell system and in normal cells. Table 77 summarizes the cell models of DBA that were analyzed.

TABLE 77
Analyzed disease models

| Normal CD34+ cells | Immortalized CD34+ cells | Primary patient cells |
|---|---|---|
| Model 1: CD34+ cells with shRNA | Model 4: Conditionally immortalized (CI) | Model 7: DBA patient cells with Dox |
| targeting RPS19 (shRPS19) | erythroblasts with Dox inducible shP53, and | inducible overexpression of RPS19 |
| | constitutive shRPS19-GFP | |
| Model 2: CD34+ cells differentiated to | Model 5: CI erythroblasts with Dox inducible | Model 8: DBA patient cells with Dox |
| erythroblasts with doxycycline (Dox) | shP53 and CRISPR removal of the RPS19 gene | inducible shP53 |
| inducible shRPS19 | | |
| Model 3: CD34+ cells differentiated to | Model 6: CI erythroblasts with Dox inducible | Model 9: CI DBA patient with Dox |
| erythroblasts with Dox inducible shP53 | overexpression of RPS19 and CRISPR removal | inducible shP53 |
| and constitutive shRPS19 | of the RPS19 gene | |

shRNAs for RPS19 were analyzed in normal CD34⁺ cells to identify shRNAs whose administration resulted in about 50% expression of RPS19. The RPS19 knockdown cell systems were treated with compounds to rescue the DBA phenotype, and the dose of these compounds were titrated to optimize the concentration.

A. Optimizing Viral Titers

HEK cell lines 293FT, 293T, and 293XT were transfected to determine which of these cell lines resulted in enhanced viral production. GFP expression was observed in all transfected cells. Viruses from each cell line were collected and tested for infectiousness. GFP expression was observed in samples of the 293XT cell line each transfected with a virus produced in one of the cell lines. GFP expression was observed in only 293XT and 293T cells and not in 293FT. In fact, virus produced in 293XT cells appeared to be the most infectious. Virus from 293FT was determined not to be infectious. Therefore 293XT cells were selected for virus production onward.

### Example 17. Effects of Different shRNAs on Gene Expression in Normal CD34⁺ Cells

shRNAs targeting RPS19, p53, or RPL11 were delivered to normal cord blood erythroblast cells using either the pLKO (constitutive) and pTRIPZ (inducible) plasmids described in Example 1. The mRNA expression of the p53 pathway proteins; progenitor factor GATA2; and differentiation factors HBB and GATA1 were measured by qRT-PCR. Cells not transduced with a shRNA as well as cells transduced with a non-targeting shRNA served as controls.

A. Effects of Constitutive Knockdown

HBB is associated with differentiation of CD34⁺ cells, and p21 is associated with the p53 pathway. Table 78 shows the relative quantification of expression levels of HBB, p21, p53, RPL11, and RPS19 in cells transduced with plasmids pLKO-p53_sh4, pLKO_p53_sh5, pLKO RPS19_1, pLKO RPS19_2, pLKO RPS19_3, pLKO RPS19_4, and pLKO_RPS19_5 compared to levels in a control pLKO-sNT. GAPDH was used as an internal control.

TABLE 78
Relative quantification of expression levels
of HBB, p21, p53, RPL11, and RPS19

| Sample | Target | CT | RQ | min | max |
|---|---|---|---|---|---|
| RPS19_sh1 | GAPDH | 24.19 | | | |
| RPS19_sh1 | RPS19 | 25.55 | 0.59 | 0.58 | 0.6 |
| RPS19_sh1 | GAPDH | 24.51 | | | |
| RPS19_sh1 | P53 | 27.84 | 1.34 | 1.14 | 1.56 |
| RPS19_sh1 | GAPDH | 24.58 | | | |
| RPS19_sh1 | RPL11 | 27.57 | 0.74 | 0.68 | 0.81 |
| RPS19_sh1 | GAPDH | 24.79 | | | |
| RPS19_sh1 | HBB | 30.11 | 0.04 | 0.04 | 0.05 |
| RPS19_sh2 | GAPDH | 22.14 | | | |
| RPS19_sh2 | RPS19 | 23.84 | 0.46 | 0.45 | 0.48 |
| RPS19_sh2 | GAPDH | 22.46 | | | |
| RPS19_sh2 | P53 | 25.89 | 1.25 | 1.18 | 1.33 |
| RPS19_sh2 | GAPDH | 22.55 | | | |
| RPS19_sh2 | RPL11 | 25.27 | 0.9 | 0.77 | 1.04 |
| RPS19_sh2 | GAPDH | 22.39 | | | |
| RPS19_sh2 | HBB | 23.53 | 0.79 | 0.78 | 0.81 |
| RPS19_sh3 | GAPDH | 26.18 | | | |
| RPS19_sh3 | RPS19 | 26.92 | 0.91 | 0.81 | 1.02 |
| RPS19_sh3 | GAPDH | 26.5 | | | |
| RPS19_sh3 | P53 | 30.03 | 1.17 | 0.9 | 1.52 |
| RPS19_sh3 | GAPDH | 26.48 | | | |
| RPS19_sh3 | RPL11 | 29.38 | 0.79 | 0.69 | 0.9 |
| RPS19_sh3 | GAPDH | 26.52 | | | |
| RPS19_sh3 | HBB | 29.93 | 0.16 | 0.14 | 0.19 |
| RPS19_sh4 | GAPDH | 26.99 | | | |
| RPS19_sh4 | RPS19 | 26.97 | 1.54 | 1.5 | 1.59 |
| RPS19_sh4 | GAPDH | 27.53 | | | |
| RPS19_sh4 | P53 | 30.98 | 1.24 | 1.19 | 1.28 |
| RPS19_sh4 | GAPDH | 27.7 | | | |
| RPS19_sh4 | RPL11 | 30.23 | 1.02 | 0.91 | 1.14 |
| RPS19_sh4 | GAPDH | 27.64 | | | |
| RPS19_sh4 | HBB | 29.69 | 0.42 | 0.39 | 0.45 |
| RPS19_sh5 | GAPDH | 23.02 | | | |
| RPS19_sh5 | RPS19 | 24.24 | 0.65 | 0.59 | 0.72 |
| RPS19_sh5 | GAPDH | 23.48 | | | |
| RPS19_sh5 | P53 | 26.74 | 1.41 | 1.22 | 1.64 |
| RPS19_sh5 | GAPDH | 23.6 | | | |
| RPS19_sh5 | RPL11 | 26.04 | 1.09 | 1.04 | 1.14 |
| RPS19_sh5 | GAPDH | 23.41 | | | |
| RPS19_sh5 | HBB | 24.63 | 0.75 | 0.71 | 0.78 |
| shNT | GAPDH | 21 | | | |
| shNT | RPS19 | 21.6 | 1 | 0.94 | 1.07 |
| shNT | GAPDH | 21.64 | | | |
| shNT | P53 | 25.4 | 1 | 0.94 | 1.07 |
| shNT | GAPDH | 21.66 | | | |
| shNT | RPL11 | 24.22 | 1 | 0.97 | 1.04 |
| shNT | GAPDH | 21.35 | | | |
| shNT | HBB | 22.15 | 1 | 0.94 | 1.07 |
| p53_sh4 | GAPDH | 22.74 | | | |
| p53_sh4 | RPS19 | 22.9 | 1.36 | 1.34 | 1.38 |
| p53_sh4 | GAPDH | 23.06 | | | |
| p53_sh4 | P53 | 29.97 | 0.11 | 0.09 | 0.14 |
| p53_sh4 | GAPDH | 23.06 | | | |
| p53_sh4 | RPL11 | 25.73 | 0.92 | 0.89 | 0.96 |
| p53_sh4 | GAPDH | 22.82 | | | |
| p53_sh4 | HBB | 24.38 | 0.59 | 0.58 | 0.61 |
| p53_sh5 | GAPDH | 22.44 | | | |
| p53_sh5 | RPS19 | 22.91 | 1.09 | 1.04 | 1.15 |
| p53_sh5 | GAPDH | 22.46 | | | |
| p53_sh5 | P53 | 30.77 | 0.04 | 0.02 | 0.08 |
| p53_sh5 | GAPDH | 22.3 | | | |
| p53_sh5 | RPL11 | 24.72 | | | |
| p53_sh5 | GAPDH | 22.09 | | | |
| p53_sh5 | HBB | 20.98 | 3.75 | 3.66 | 3.84 |

RPS19 knockdown was observed to lead to consistent activation of the p53 pathway and decreased expression of the differentiation factors HBB and GATA1, as shown in Table 78. Cells transduced with pLKO_p53_sh4 or pLKO_p53_sh5 with an shRNA targeting p53 had a reduction in expression of p53 compared to the control. For cells transduced with pLKO_p53_sh3, expression of other the other genes analyzed was unchanged.

To determine the effect of p53 knockdown on expression of other genes, cells were also transduced with pLKO containing p53_sh4 or p53_sh5. To determine the effect of RPS19 knockdown on expression of other genes, cells were transduced with pLKO containing RPS19_sh1, RPS19_sh2, RPS19_sh3, RPS19_sh4, RPS19_sh5, and sh_NT as a control. The relative quantifications of expression of differentiation associated genes ALAS2 and HBB, immortalization factors BCLXL and MYC, and targets of interest p53 and RPS19 are shown in Table 79.

TABLE 79
Relative quantification of expression levels
of ALAS2, HBB, BCLXL, MYC, p53, and RPS19

| Sample | Target | CT | RQ | min | max |
|---|---|---|---|---|---|
| p53_sh4 | RPS19 | 22.48 | 0.48 | 0.40 | 0.59 |
| p53_sh4 | p53 | 29.65 | 0.14 | 0.11 | 0.17 |
| p53_sh4 | HBB | 25.19 | 0.36 | 0.33 | 0.40 |
| p53_sh4 | ALAS2 | 26.75 | 1.64 | 1.45 | 1.86 |
| p53_sh4 | MYC | 24.33 | 1.22 | 1.11 | 1.34 |
| p53_sh4 | BCLXL | 23.01 | 1.01 | 0.91 | 1.12 |
| p53_sh4 | p21 | 27.38 | 8.71 | 7.75 | 9.79 |
| p53_sh4 | B-ACTIN | 24.26 | | | |
| p53_sh5 | RPS19 | 21.45 | 0.44 | 0.39 | 0.49 |
| p53_sh5 | p53 | 29.82 | 0.05 | 0.04 | 0.08 |
| p53_sh5 | HBB | 20.07 | 5.56 | 4.56 | 6.77 |
| p53_sh5 | ALAS2 | 24.38 | 3.78 | 3.39 | 4.21 |
| p53_sh5 | MYC | 22.73 | 1.64 | 1.43 | 1.89 |
| p53_sh5 | BCLXL | 21.52 | 1.26 | 1.12 | 1.42 |
| p53_sh5 | p21 | 32.53 | 0.11 | 0.09 | 0.14 |
| p53_sh5 | B-ACTIN | 23.09 | | | |
| RPS19_sh1 | RPS19 | 21.13 | 0.47 | 0.37 | 0.60 |
| RPS19_sh1 | p53 | 26.04 | 0.65 | 0.52 | 0.81 |
| RPS19_sh1 | HBB | 30.86 | 0.00 | 0.00 | 0.00 |
| RPS19_sh1 | ALAS2 | 31.90 | 0.02 | 0.02 | 0.02 |
| RPS19_sh1 | MYC | 24.19 | 0.52 | 0.46 | 0.59 |
| RPS19_sh1 | BCLXL | 23.20 | 0.34 | 0.31 | 0.37 |
| RPS19_sh1 | p21 | 30.03 | 0.53 | 0.49 | 0.58 |
| RPS19_sh1 | B-ACTIN | 22.88 | | | |
| RPS19_sh2 | RPS19 | 20.80 | 0.52 | 0.44 | 0.61 |
| RPS19_sh2 | p53 | 24.99 | 1.18 | 1.05 | 1.32 |
| RPS19_sh2 | HBB | 25.39 | 0.11 | 0.09 | 0.12 |
| RPS19_sh2 | ALAS2 | 28.14 | 0.21 | 0.17 | 0.27 |
| RPS19_sh2 | MYC | 23.60 | 0.68 | 0.66 | 0.70 |
| RPS19_sh2 | BCLXL | 22.80 | 0.39 | 0.35 | 0.45 |
| RPS19_sh2 | p21 | 29.48 | 0.68 | 0.60 | 0.78 |
| RPS19_sh2 | B-ACTIN | 22.68 | | | |
| RPS19_sh3 | RPS19 | 22.50 | 0.42 | 0.34 | 0.52 |
| RPS19_sh3 | p53 | 26.66 | 0.97 | 0.54 | 1.73 |
| RPS19_sh3 | HBB | 32.13 | 0.00 | 0.00 | 0.00 |
| RPS19_sh3 | ALAS2 | 33.78 | 0.01 | 0.01 | 0.01 |
| RPS19_sh3 | MYC | 25.40 | 0.51 | 0.45 | 0.58 |
| RPS19_sh3 | BCLXL | 24.14 | 0.41 | 0.35 | 0.48 |
| RPS19_sh3 | p21 | 30.24 | 1.06 | 0.92 | 1.21 |
| RPS19_sh3 | B-ACTIN | 24.08 | | | |
| RPS19_sh4 | RPS19 | 27.49 | 0.43 | 0.33 | 0.56 |
| RPS19_sh4 | p53 | 30.85 | 1.74 | 1.23 | 2.47 |
| RPS19_sh4 | HBB | 33.64 | 0.03 | 0.02 | 0.04 |
| RPS19_sh4 | ALAS2 | 34.96 | 0.16 | 0.13 | 0.20 |
| RPS19_sh4 | MYC | 29.42 | 1.03 | 0.85 | 1.26 |
| RPS19_sh4 | BCLXL | 28.08 | 0.87 | 0.70 | 1.08 |
| RPS19_sh4 | p21 | 31.68 | 12.78 | 10.25 | 15.92 |
| RPS19_sh4 | B-ACTIN | 29.11 | | | |
| RPS19_sh5 | RPS19 | 23.80 | 0.15 | 0.12 | 0.18 |
| RPS19_sh5 | p53 | 26.84 | 0.74 | 0.63 | 0.88 |
| RPS19_sh5 | HBB | 26.09 | 0.15 | 0.12 | 0.17 |
| RPS19_sh5 | ALAS2 | 26.48 | 1.51 | 1.27 | 1.78 |
| RPS19_sh5 | MYC | 23.70 | 1.43 | 1.16 | 1.77 |
| RPS19_sh5 | BCLXL | 22.64 | 0.99 | 0.84 | 1.17 |
| RPS19_sh5 | p21 | 27.58 | 5.75 | 4.72 | 7.00 |
| RPS19_sh5 | B-ACTIN | 23.86 | | | |
| sh_NT | RPS19 | 19.98 | 1.00 | 0.77 | 1.29 |
| sh_NT | p53 | 25.35 | 1.00 | 0.51 | 1.96 |
| sh_NT | HBB | 22.27 | 1.00 | 0.93 | 1.07 |
| sh_NT | ALAS2 | 26.02 | 1.00 | 0.87 | 1.15 |
| sh_NT | MYC | 23.17 | 1.00 | 0.89 | 1.12 |
| sh_NT | BCLXL | 21.58 | 1.00 | 0.67 | 1.49 |
| sh_NT | p21 | 29.05 | 1.00 | 0.93 | 1.08 |
| sh_NT | B-ACTIN | 22.81 | | | |

RPS19_sh4 and RPS19_sh5 were observed to result in a 12.8 and 5.8-fold increase in expression of p21, respectively compared to the level of expression observed for the p53 knockdowns.

B. Effects of Inducible Knockdown

Hematopoietic progenitor CD34⁺ cells were transduced with the pTRIPZ inducible lentiviral plasmids containing p53_sh1, puro_shNT, RPL11_1, RPL11_2, RPS19_1, or RPS19_2. Table 80 shows the relative quantification of expression levels of HBB, p21, p53, RPL11, and RPS19 in cells transduced with inducible pTRIPZ.

TABLE 80
Relative quantification of expression levels of HBB, p21, p53, RPL11, and RPS19

| Sample | Target | CT | RQ | min | max | Sample | Target | CT | RQ | min | max |
|---|---|---|---|---|---|---|---|---|---|---|---|
| RPS19_2 | p53 | 25.24 | 1.12 | 0.96 | 1.31 | RPL11_1 | p53 | 24.70 | 2.31 | 1.91 | 2.80 |
| RPS19_2 | RPL11 | 20.55 | 1.46 | 1.09 | 1.96 | RPL11_1 | RPL11 | 23.75 | 0.23 | 0.15 | 0.35 |
| RPS19_2 | RPS19 | 20.73 | 0.77 | 0.66 | 0.89 | RPL11_1 | RPS19 | 19.66 | 2.30 | 1.76 | 3.00 |
| RPS19_2 | p21 | 26.82 | 1.73 | 1.37 | 2.19 | RPL11_1 | p21 | 23.66 | 22.15 | 17.89 | 27.43 |
| RPS19_2 | HBB | 25.88 | 0.55 | 0.45 | 0.67 | RPL11_1 | HBB | 26.44 | 0.53 | 0.45 | 0.63 |
| RPS19_2 | B-ACTIN | 21.22 | | | | RPL11_1 | B-ACTIN | 21.73 | | | |
| RPS19_1 | p53 | 25.12 | 1.66 | 1.27 | 2.17 | puro_shNT | p53 | 25.25 | 1.00 | 0.85 | 1.18 |
| RPS19_1 | RPL11 | 20.62 | 1.90 | 1.32 | 2.74 | puro_shNT | RPL11 | 20.94 | 1.00 | 0.80 | 1.25 |
| RPS19_1 | RPS19 | 21.89 | 0.47 | 0.41 | 0.54 | puro_shNT | RPS19 | 20.20 | 1.00 | 0.82 | 1.22 |
| RPS19_1 | p21 | 25.51 | 5.87 | 4.31 | 7.98 | puro_shNT | p21 | 27.46 | 1.00 | 0.74 | 1.36 |
| RPS19_1 | HBB | 26.47 | 0.50 | 0.40 | 0.63 | puro_shNT | HBB | 24.87 | 1.00 | 0.78 | 1.28 |
| RPS19_1 | B-ACTIN | 21.67 | | | | puro_shNT | B-ACTIN | 21.07 | | | |

| Sample | Target | CT | RQ | min | max |
|---|---|---|---|---|---|
| RPL11_2 | p53 | 25.52 | 2.35 | 1.91 | 2.88 |
| RPL11_2 | RPL11 | 23.93 | 0.36 | 0.27 | 0.47 |
| RPL11_2 | RPS19 | 19.86 | 3.58 | 3.22 | 3.99 |
| RPL11_2 | p21 | 24.62 | 20.43 | 15.77 | 26.46 |
| RPL11_2 | HBB | 26.79 | 0.75 | 0.67 | 0.84 |
| RPL11_2 | B-ACTIN | 22.57 | | | |
| p53_sh1 | p53 | 26.40 | 0.43 | 0.35 | 0.53 |
| p53_sh1 | RPL11 | 20.90 | 0.98 | 0.84 | 1.14 |
| p53_sh1 | RPS19 | 20.09 | 1.02 | 0.87 | 1.20 |
| p53_sh1 | p21 | 27.42 | 0.98 | 0.80 | 1.20 |
| p53_sh1 | HBB | 25.25 | 0.73 | 0.59 | 0.91 |
| p53_sh1 | B-ACTIN | 20.99 | | | |

Cells transduced with sh1 demonstrated lower levels of RPS19 mRNA compared to non-transduced cells. This showed that the pTRIPZ inducible shRNA was working efficiently. Expression of p21 was significantly increased in cells transduced with RPL11 RNA knockdown vectors pTRIPZ_RPL11_1 and pTRIPZ_RPL11_2, confirming the DBA-like phenotype.

The relative expression of GATA1, GATA2, MDM2, BAX, and BAG1 were also analyzed in cells transduced with a pTRIPZ dox inducible shRNA. Results are shown in Table 81. B-Actin was used as an internal control.

TABLE 81
Relative quantification of expression levels

| Sample | Target | CT | RQ | min | max | Sample | Target | CT | RQ | min | max |
|---|---|---|---|---|---|---|---|---|---|---|---|
| p53_sh1 | GATA1 | 24.14 | 0.91 | 0.76 | 1.08 | RPL11_2 | GATA1 | 26.09 | 0.71 | 0.66 | 0.76 |
| p53_sh1 | GATA2 | 24.51 | 0.97 | 0.85 | 1.11 | RPL11_2 | GATA2 | 25.80 | 1.21 | 1.01 | 1.46 |
| p53_sh1 | MDM2 | 25.47 | 0.84 | 0.76 | 0.93 | RPL11_2 | MDM2 | 24.92 | 3.75 | 3.48 | 4.03 |
| p53_sh1 | BAX | 23.46 | 0.91 | 0.70 | 1.18 | RPL11_2 | BAX | 23.67 | 2.40 | 1.98 | 2.90 |
| p53_sh1 | BAG1 | 25.11 | 1.04 | 0.92 | 1.18 | RPL11_2 | BAG1 | 25.97 | 1.75 | 1.61 | 1.90 |
| p53_sh1 | B-ACTIN | 20.93 | | | | RPL11_2 | B-ACTIN | 22.53 | | | |
| puro_shNT | GATA1 | 24.08 | 1.00 | 0.89 | 1.12 | RPS19_1 | GATA1 | 25.16 | 0.67 | 0.60 | 0.76 |
| puro_shNT | GATA2 | 24.56 | 1.00 | 0.83 | 1.20 | RPS19_1 | GATA2 | 24.46 | 1.53 | 1.24 | 1.89 |
| puro_shNT | MDM2 | 25.31 | 1.00 | 0.86 | 1.17 | RPS19_1 | MDM2 | 24.37 | 2.72 | 2.41 | 3.08 |
| puro_shNT | BAX | 23.41 | 1.00 | 0.86 | 1.16 | RPS19_1 | BAX | 23.27 | 1.57 | 1.32 | 1.87 |
| puro_shNT | BAG1 | 25.26 | 1.00 | 0.86 | 1.16 | RPS19_1 | BAG1 | 25.55 | 1.16 | 1.03 | 1.31 |
| puro_shNT | B-ACTIN | 21.01 | | | | RPS19_1 | B-ACTIN | 21.53 | | | |
| RPL11_1 | GATA1 | 25.49 | 0.58 | 0.51 | 0.66 | RPS19_2 | GATA1 | 24.43 | 0.87 | 0.77 | 0.99 |
| RPL11_1 | GATA2 | 25.01 | 1.12 | 0.96 | 1.31 | RPS19_2 | GATA2 | 24.42 | 1.23 | 0.99 | 1.51 |
| RPL11_1 | MDM2 | 24.32 | 3.04 | 2.64 | 3.50 | RPS19_2 | MDM2 | 25.00 | 1.38 | 1.17 | 1.62 |
| RPL11_1 | BAX | 23.37 | 1.58 | 1.41 | 1.77 | RPS19_2 | BAX | 23.20 | 1.29 | 1.18 | 1.41 |
| RPL11_1 | BAG1 | 25.63 | 1.18 | 1.01 | 1.39 | RPS19_2 | BAG1 | 25.31 | 1.08 | 0.93 | 1.25 |
| RPL11_1 | B-ACTIN | 21.63 | | | | RPS19_2 | B-ACTIN | 21.17 | | | |

Among the p53 pathway proteins analyzed, sh1 transduction lead to a higher expression of BAX, P21, P53, and MDM2 mRNA, compared to sh2 transduction. Additionally, the mRNA expression levels of the progenitor protein GATA2 were higher upon sh1 transduction compared to sh2 transduction. mRNA levels of the differentiation associated proteins HBB and GATA1 were higher following sh2 transduction compared to their levels upon sh1 transduction.

C. Reversion of Mortality

Early erythroblast progenitor cells derived from cord blood were transduced using lentiviral vectors harboring overexpression constructs of apoptosis-associated genes, BCLXL and MYC, under the control of a doxycycline inducible promoter. The mRNA expression of the differentiation markers ALAS2, HBB and the targets of interest, namely, p53 and RPS19 were measured using qRT-PCR, and results are shown in Table 82.

TABLE 82
Relative quantification of expression of RPS19, p53, HBB, ALAS2, MYC, BCLXL, and p21

| Sample | Target | CT | RQ | min | max | Sample | Target | CT | RQ | min | max |
|---|---|---|---|---|---|---|---|---|---|---|---|
| D2-dox+ | RPS19 | 25.67 | 1.00 | 0.88 | 1.13 | D4-Dox− | RPS19 | 23.43 | 0.28 | 0.22 | 0.36 |
| D2-dox+ | p53 | 29.60 | 1.00 | 0.87 | 1.15 | D4-Dox− | p53 | 27.72 | 0.22 | 0.16 | 0.30 |
| D2-dox+ | HBB | 26.81 | 1.00 | 0.83 | 1.20 | D4-Dox− | HBB | 20.46 | 4.89 | 3.94 | 6.07 |
| D2-dox+ | ALAS2 | 27.74 | 1.00 | 0.75 | 1.33 | D4-Dox− | ALAS2 | 23.77 | 0.94 | 0.72 | 1.22 |
| D2-dox+ | MYC | 25.80 | 1.00 | 0.82 | 1.21 | D4-Dox− | MYC | 25.81 | 0.06 | 0.05 | 0.07 |
| D2-dox+ | BCLXL | 24.91 | 1.00 | 0.85 | 1.17 | D4-Dox− | BCLXL | 24.72 | 0.07 | 0.05 | 0.09 |
| D2-dox+ | p21 | 30.93 | 1.00 | 0.86 | 1.16 | D4-Dox− | p21 | 25.85 | 2.03 | 1.59 | 2.59 |
| D2-dox+ | B-ACTIN | 27.17 | | | | D4-Dox− | B-ACTIN | 23.11 | | | |
| D2-Dox− | RPS19 | 27.10 | 0.28 | 0.26 | 0.30 | D5-Dox− | RPS19 | 22.88 | 0.27 | 0.22 | 0.33 |
| D2-Dox− | p53 | 30.50 | 0.40 | 0.29 | 0.56 | D5-Dox− | p53 | 27.25 | 0.20 | 0.16 | 0.25 |
| D2-Dox− | HBB | 23.42 | 7.81 | 7.24 | 8.44 | D5-Dox− | HBB | 21.03 | 2.13 | 1.76 | 2.58 |
| D2-Dox− | ALAS2 | 25.75 | 2.98 | 2.55 | 3.48 | D5-Dox− | ALAS2 | 24.38 | 0.40 | 0.30 | 0.53 |
| D2-Dox− | MYC | 29.96 | 0.04 | 0.04 | 0.04 | D5-Dox− | MYC | 25.23 | 0.06 | 0.05 | 0.07 |
| D2-Dox− | BCLXL | 26.29 | 0.29 | 0.24 | 0.35 | D5-Dox− | BCLXL | 24.83 | 0.04 | 0.03 | 0.05 |
| D2-Dox− | p21 | 29.30 | 2.31 | 1.85 | 2.88 | D5-Dox− | p21 | 25.56 | 1.61 | 1.25 | 2.07 |
| D2-Dox− | B-ACTIN | 26.75 | | | | D5-Dox− | B-ACTIN | 22.49 | | | |
| D3-Dox− | RPS19 | 23.47 | 0.21 | 0.16 | 0.28 | D6-dox− | RPS19 | 23.57 | 0.21 | 0.17 | 0.26 |
| D3-Dox− | p53 | 27.43 | 0.21 | 0.16 | 0.27 | D6-dox− | p53 | 27.91 | 0.16 | 0.14 | 0.19 |
| D3-Dox− | HBB | 19.89 | 5.57 | 4.33 | 7.16 | D6-dox− | HBB | 23.28 | 0.57 | 0.51 | 0.64 |
| D3-Dox− | ALAS2 | 22.77 | 1.44 | 1.06 | 1.97 | D6-dox− | ALAS2 | 25.99 | 0.17 | 0.15 | 0.19 |
| D3-Dox− | MYC | 26.68 | 0.03 | 0.02 | 0.03 | D6-dox− | MYC | 26.32 | 0.03 | 0.03 | 0.04 |
| D3-Dox− | BCLXL | 23.70 | 0.11 | 0.08 | 0.14 | D6-dox− | BCLXL | 25.77 | 0.03 | 0.02 | 0.03 |
| D3-Dox− | p21 | 26.42 | 1.05 | 0.78 | 1.43 | D6-dox− | p21 | 26.41 | 1.14 | 0.96 | 1.37 |
| D3-Dox− | B-ACTIN | 22.74 | | | | D6-dox− | B-ACTIN | 22.85 | | | |

The mRNA expression of MYC and BCLXL was highest upon doxycycline induction and decreased 2, 3, 4, 5, and 6 days after doxycycline was withdrawn. The mRNA expression of the differentiation markers ALAS2 and HBB increased immediately after doxycycline was withdrawn and gradually decreased up to day 6 of doxycycline withdrawal. The mRNA expression pattern of MYC and BCLXL sharply decreased 2 days after doxycycline was withdrawn as shown in Table 82. The decrease in the mRNA expression of the immortalization factors correlated with an increase in the mRNA expression of the differentiation factors. This confirms that immortality can quickly be reversed upon system shut off via retracting immortalization factors.

D. Comparing Cell Size after Removal of Dox in Cells Transduced with Model 2

Cell size is shown in Table 83 during doxycycline induction, and immediately following withdrawal of doxycycline. Conditionally immortalized cells maintained in the presence of doxycycline were observed to have a cell size between 12-13.5 μm. By day 5 off doxycycline cells dramatically decreased in size to approximately 9 μm, which demonstrates a reversion of mortality and differentiation to late erythroblast stage.

TABLE 83
Cell size

| Days on DOX | Cell size (μM) | Days on DOX | Cell size (μM) | Days off DOX | Cell size (μM) | Days off DOX | Cell size (μM) |
|---|---|---|---|---|---|---|---|
| 2 | 12.6 | 5 | 12.7 | 2 | 9.7 | 5 | 9.0 |
| 3 | 13.3 | 6 | 12.0 | 3 | 10.5 | 6 | 9.1 |
| 4 | 13.5 | | | 4 | 10.1 | | |

E. Effects of Knockdown of Both RPS19 and shP53

Further qRT-PCR analysis of the parental cord blood IRES cell line with Dox inducible p53 (CB-IRES-p53-GFPtrad) that were separated using turbo-GFP magnetic beads is shown in Table 84. GAPDH was used as an internal control.

TABLE 84
Percent of knockdown

| | NT(shP53) | shRPS19_1 + shP53 | shRPS19_2 + shP53 |
|---|---|---|---|
| p53 | 4 | 4 | 4 |
| RPS19 | 106 | 47 | 49 |

The cells administered the shRPS19 were observed to have an about 50% knockdown of RPS19, while p53 remained constant. p53 remains constant due to the NT control expressing the shRNA for p53. p53 is completely reduced in all three lines. The RPS19 shRNA constructs were normalized to the NT (shp53) control.

### Example 18. Effects of Different shRNAs on Gene Expression in Conditionally Immortalized Cells

qRT-PCR was used to analyze expression of p53 pathway-associated genes p21, RPS19, and RPL11; differentiation marker HBB; and internal control B-ACTIN in the cord blood conditionally immortalized CD34+ (CB-CI-IRES-CD34+) cell line transduced with the pTRIPZ plasmid containing NT (Model 9), RPL11_sh1 (Model 4) and RPL11_sh2 (Model 5) for a RPL11 knockdown, and RPS19_sh1 (Model 6) and RPS19_sh2 (Model 7) for a RPS19 knockdown. Results are shown in Table 85.

TABLE 85
Relative quantification of gene expression of RPS19, RPL11, p21, and HBB

| Sample | Target | CT | RQ | min | max | Sample | Target | CT | RQ | min | max |
|---|---|---|---|---|---|---|---|---|---|---|---|
| pTRIPZ NT | RPS19 | 20.23 | 1.00 | 0.90 | 1.12 | RPS19 sh1 | RPS19 | 22.51 | 0.30 | 0.21 | 0.41 |
| pTRIPZ_NT | RPL11 | 20.98 | 1.00 | 0.88 | 1.13 | RPS19_sh1 | RPL11 | 20.91 | 1.52 | 1.10 | 2.08 |
| pTRIPZ_NT | p21 | 25.78 | 1.00 | 0.83 | 1.20 | RPS19_sh1 | p21 | 25.87 | 1.35 | 0.95 | 1.94 |
| pTRIPZ NT | HBB | 25.78 | 1.00 | 0.79 | 1.27 | RPS19_sh1 | HBB | 25.11 | 2.28 | 1.64 | 3.17 |
| pTRIPZ_NT | B-ACTIN | 21.59 | | | | RPS19_sh1 | B-ACTIN | 22.12 | | | |
| RPL11_sh1 | RPS19 | 21.99 | 1.48 | 1.25 | 1.74 | RPS19_sh2 | RPS19 | 21.46 | 0.46 | 0.38 | 0.54 |
| RPL11_sh1 | RPL11 | 22.94 | 1.29 | 0.50 | 3.31 | RPS19_sh2 | RPL11 | 20.78 | 1.23 | 1.07 | 1.42 |
| RPL11_sh1 | p21 | 25.80 | 4.96 | 3.96 | 6.22 | RPS19_sh2 | p21 | 26.16 | 0.82 | 0.59 | 1.13 |
| RPL11_sh1 | HBB | 27.01 | 2.14 | 1.74 | 2.64 | RPS19_sh2 | HBB | 25.33 | 1.46 | 1.14 | 1.88 |
| RPL11_sh1 | B-ACTIN | 23.92 | | | | RPS19_sh2 | B-ACTIN | 21.69 | | | |
| RPL11_sh2 | RPS19 | 21.45 | 1.28 | 1.09 | 1.50 | | | | | | |
| RPL11_sh2 | RPL11 | 22.83 | 0.83 | 0.72 | 0.95 | | | | | | |
| RPL11_sh2 | p21 | 25.93 | 2.68 | 2.17 | 3.32 | | | | | | |
| RPL11_sh2 | HBB | 27.33 | 1.02 | 0.88 | 1.19 | | | | | | |
| RPL11_sh2 | B-ACTIN | 23.17 | | | | | | | | | |

A. Comparing Gene Expression Levels During Expansion and Differentiation

qRT-PCR was used to determine the levels of expression of ALAS2, GAPDH, GATA1, GATA2, HBB, and p53 in differentiated CB-CI-IRES cells using GAPDH as an internal control as shown in Table 86.

TABLE 86
Relative quantification of expression of GATA1, GATA2, HBB, ALAS2, and p53

| Sample | Target | CT | RQ | min | max | Sample | Target | CT | RQ | min | max |
|---|---|---|---|---|---|---|---|---|---|---|---|
| p53sh1-DIFF | GAPDH | 19.66 | | | | p53sh3-DIFF | GAPDH | 20.68 | | | |
| p53sh1-DIFF | GATA1 | 23.94 | 1.47 | 1.44 | 1.50 | p53sh3-DIFF | GATA1 | 23.88 | 3.09 | 2.87 | 3.32 |
| p53sh1-DIFF | GAPDH | 20.00 | | | | p53sh3-DIFF | GAPDH | 20.44 | | | |
| p53sh1-DIFF | GATA2 | 24.09 | 0.34 | 0.06 | 1.81 | p53sh3-DIFF | GATA2 | 25.03 | 0.24 | 0.23 | 0.25 |
| p53sh1-DIFF | GAPDH | 19.60 | | | | p53sh3-DIFF | GAPDH | 21.76 | | | |
| p53sh1-DIFF | HBB | 24.03 | 2.40 | 2.27 | 2.54 | p53sh3-DIFF | HBB | 24.91 | 5.83 | 5.42 | 6.27 |
| p53sh1-DIFF | GAPDH | 19.90 | | | | p53sh3-DIFF | GAPDH | 20.29 | | | |
| p53sh1-DIFF | ALAS2 | 22.52 | 4.83 | 2.38 | 9.83 | p53sh3-DIFF | ALAS2 | 22.28 | 7.52 | 6.99 | 8.09 |
| p53sh1-DIFF | GAPDH | 19.77 | | | | p53sh3-DIFF | GAPDH | 20.37 | | | |
| p53sh1-DIFF | p53 | 28.29 | 0.12 | 0.11 | 0.13 | p53sh3-DIFF | p53 | 28.93 | 0.12 | 0.10 | 0.14 |
| p53sh2-DIFF | GAPDH | 19.55 | | | | shNT-DIFF | GAPDH | 20.07 | | | |
| p53sh2-DIFF | GATA1 | 24.14 | 1.18 | 1.15 | 1.20 | shNT-DIFF | GATA1 | 24.90 | 1.00 | 0.97 | 1.03 |
| p53sh2-DIFF | GAPDH | 19.73 | | | | shNT-DIFF | GAPDH | 20.71 | | | |
| p53sh2-DIFF | GATA2 | 23.95 | 0.31 | 0.28 | 0.34 | shNT-DIFF | GATA2 | 23.23 | 1.00 | 0.79 | 1.27 |
| p53sh2-DIFF | GAPDH | 19.75 | | | | shNT-DIFF | GAPDH | 20.02 | | | |
| p53sh2-DIFF | HBB | 25.37 | 1.06 | 0.93 | 1.22 | shNT-DIFF | HBB | 25.72 | 1.00 | 0.87 | 1.15 |
| p53sh2-DIFF | GAPDH | 19.50 | | | | shNT-DIFF | GAPDH | 20.45 | | | |
| p53sh2-DIFF | ALAS2 | 22.76 | 3.11 | 2.68 | 3.61 | shNT-DIFF | ALAS2 | 25.35 | 1.00 | 0.94 | 1.06 |
| p53sh2-DIFF | GAPDH | 19.62 | | | | shNT-DIFF | GAPDH | 20.09 | | | |
| p53sh2-DIFF | p53 | 28.37 | 0.10 | 0.09 | 0.11 | shNT-DIFF | p53 | 25.54 | 1.00 | 0.92 | 1.08 |

Gene expression was compared for CB-CI-IRES cells during expansion and differentiation. Table 87 shows the differences in expression of differentiation markers GATA1, GATA2, and HBB and p53 in erythroid cells transduced with different lentivirus resulting in a p53 knockdown. GAPDH was used as an internal control.

TABLE 87
Relative quantification of expression levels of GATA1, GATA2, HBB, and p5

| Sample | Target | CT | RQ | min | max | Sample | Target | CT | RQ | min | max |
|---|---|---|---|---|---|---|---|---|---|---|---|
| p53sh1-Exp | GAPDH | 19.89 | | | | p53sh2-DIFF | GAPDH | 19.55 | | | |
| p53sh1-Exp | GATA1 | 24.09 | 1.55 | 1.42 | 1.69 | p53sh2-DIFF | GATA1 | 24.14 | 1.18 | 1.15 | 1.20 |
| p53sh1-Exp | GAPDH | 20.42 | | | | p53sh2-DIFF | GAPDH | 19.73 | | | |
| p53sh1-Exp | GATA2 | 23.55 | 0.66 | 0.38 | 1.14 | p53sh2-DIFF | GATA2 | 23.95 | 0.31 | 0.28 | 0.34 |
| p53sh1-Exp | GAPDH | 19.82 | | | | p53sh2-DIFF | GAPDH | 19.75 | | | |
| p53sh1-Exp | HBB | 20.44 | 33.75 | 31.73 | 35.91 | p53sh2-DIFF | HBB | 25.37 | 1.06 | 0.93 | 1.22 |
| p53sh1-Exp | GAPDH | 20.25 | | | | p53sh2-DIFF | GAPDH | 19.50 | | | |
| p53sh1-Exp | ALAS2 | 22.66 | 5.61 | 4.27 | 7.37 | p53sh2-DIFF | ALAS2 | 22.76 | 3.11 | 2.68 | 3.61 |
| p53sh1-Exp | GAPDH | 20.04 | | | | p53sh2-DIFF | GAPDH | 19.62 | | | |
| p53sh1-Exp | p53 | 28.06 | 0.17 | 0.14 | 0.20 | p53sh2-DIFF | p53 | 28.37 | 0.10 | 0.09 | 0.11 |
| shNT-Exp | GAPDH | 22.02 | | | | p53sh3-DIFF | GAPDH | 20.68 | | | |
| shNT-Exp | GATA1 | 26.80 | 1.04 | 0.97 | 1.10 | p53sh3-DIFF | GATA1 | 23.88 | 3.09 | 2.87 | 3.32 |
| shNT-Exp | GAPDH | 20.67 | | | | p53sh3-DIFF | GAPDH | 20.44 | | | |
| shNT-Exp | GATA2 | 23.61 | 0.75 | 0.54 | 1.03 | p53sh3-DIFF | GATA2 | 25.03 | 0.24 | 0.23 | 0.25 |
| shNT-Exp | GAPDH | 19.81 | | | | p53sh3-DIFF | GAPDH | 21.76 | | | |
| shNT-Exp | HBB | 22.45 | 8.34 | 8.20 | 8.48 | p53sh3-DIFF | HBB | 24.91 | 5.83 | 5.42 | 6.27 |
| shNT-Exp | GAPDH | 20.49 | | | | p53sh3-DIFF | GAPDH | 20.29 | | | |
| shNT-Exp | ALAS2 | 24.07 | 2.48 | 1.78 | 3.46 | p53sh3-DIFF | ALAS2 | 22.28 | 7.52 | 6.99 | 8.09 |
| shNT-Exp | GAPDH | 19.98 | | | | p53sh3-DIFF | GAPDH | 20.37 | | | |
| shNT-Exp | p53 | 25.89 | 0.73 | 0.59 | 0.89 | p53sh3-DIFF | p53 | 28.93 | 0.12 | 0.10 | 0.14 |

| Sample | Target | CT | RQ | min | max |
|---|---|---|---|---|---|
| p53sh1-DIFF | GAPDH | 19.66 | | | |
| p53sh1-DIFF | GATA1 | 23.94 | 1.47 | 1.44 | 1.50 |
| p53sh1-DIFF | GAPDH | 20.00 | | | |
| p53sh1-DIFF | GATA2 | 24.09 | 0.34 | 0.06 | 1.81 |
| p53sh1-DIFF | GAPDH | 19.60 | | | |
| p53sh1-DIFF | HBB | 24.03 | 2.40 | 2.27 | 2.54 |
| p53sh1-DIFF | GAPDH | 19.90 | | | |
| p53sh1-DIFF | ALAS2 | 22.52 | 4.83 | 2.38 | 9.83 |
| p53sh1-DIFF | GAPDH | 19.77 | | | |
| p53sh1-DIFF | p53 | 28.29 | 0.12 | 0.11 | 0.13 |
| shNT-DIFF | GAPDH | 20.07 | | | |
| shNT-DIFF | GATA1 | 24.90 | 1.00 | 0.97 | 1.03 |
| shNT-DIFF | GAPDH | 20.71 | | | |
| shNT-DIFF | GATA2 | 23.23 | 1.00 | 0.79 | 1.27 |
| shNT-DIFF | GAPDH | 20.02 | | | |
| shNT-DIFF | HBB | 25.72 | 1.00 | 0.87 | 1.15 |
| shNT-DIFF | GAPDH | 20.45 | | | |
| shNT-DIFF | ALAS2 | 25.35 | 1.00 | 0.94 | 1.06 |
| shNT-DIFF | GAPDH | 20.09 | | | |
| shNT-DIFF | p53 | 25.54 | 1.00 | 0.92 | 1.08 |

Mature erythroid specific genes HBB and ALAS2 were observed to be upregulated, which confirmed that the cells were differentiated.

B. Changes in Gene Expression Levels of GATA1 and GATA2 Through Day 15 of Differentiation

ChIP-seq results herein show that GATA1 and GATA2 binds genomic signaling centers in both expanding and differentiated cord blood cells. Changes in expression of GATA1 and GATA2 were further observed starting a day 10 (D10), then measured at days D1-D4, D7-D8, D10-D11, and D14-D15 of differentiation by qRT-PCR. Results are shown in Table 88.

TABLE 88
Relative quantification through cord blood cell differentiation

| Sample | Target | RQ | MAX | MIN | Target | RQ | MAX | MIN |
|---|---|---|---|---|---|---|---|---|
| D10-Exp | GATA1 | 1.00 | 0.92 | 1.09 | GATA2 | 1.00 | 0.58 | 1.72 |
| D1-Diff | GATA1 | 0.88 | 0.80 | 0.97 | GATA2 | 1.11 | 1.05 | 1.17 |
| D2-Diff | GATA1 | 1.05 | 1.02 | 1.09 | GATA2 | 0.70 | 0.53 | 0.93 |
| D3-Diff | GATA1 | 1.43 | 1.38 | 1.49 | GATA2 | 1.36 | 0.95 | 1.95 |
| D4-Diff | GATA1 | 1.87 | 1.74 | 2.02 | GATA2 | 0.67 | 0.64 | 0.70 |
| D7-Diff | GATA1 | 2.66 | 2.39 | 2.95 | GATA2 | 3.20 | 2.61 | 3.91 |
| D8-Diff | GATA1 | 5.05 | 4.77 | 5.35 | GATA2 | 0.82 | 0.77 | 0.87 |
| D8-Diff | GATA1 | 1.78 | 1.64 | 1.94 | GATA2 | 0.66 | 0.58 | 0.75 |
| D10-Diff | GATA1 | 2.35 | 2.31 | 2.39 | GATA2 | 0.34 | 0.32 | 0.37 |
| D11-Diff | GATA1 | 3.04 | 2.83 | 3.26 | GATA2 | 0.36 | 0.34 | 0.39 |
| D14-Diff | GATA1 | 3.41 | 3.27 | 3.55 | GATA2 | 0.30 | 0.27 | 0.34 |
| D15-Diff | GATA1 | 1.50 | 1.42 | 1.57 | GATA2 | 0.24 | 0.20 | 0.30 |

GATA1 was observed to increase throughout erythroid differentiation, and GATA2 was observed to decrease only at late stage erythroid differentiation.

C. Differences in Expression of Apoptotic Genes

Gene expression of differentiation marker ALAS2 and apoptotic markers BA and GADD45A were compared were compared between expansion Day 11 (Exp_D11) and Differentiation Day 7 (Diff_D7) using qRT-PCR. GAPDH was used as an internal control. Table 89 provides the qRT-PCR results.

TABLE 89
Relative quantification of ALAS2, BAX, GATA1,
GATA2, HBB, p21, p53, and GADD45A

| SAMPLE | TARGET | RQ | max | min | SAMPLE | TARGET | RQ | max | min |
|---|---|---|---|---|---|---|---|---|---|
| Diff_D7 | ALAS2 | 23.09 | 5.72 | 7.60 | Exp_D11 | ALAS2 | 1.00 | 0.21 | 0.27 |
| Diff_D7 | ALAS2 | 34.98 | 8.28 | 10.85 | Exp_D11 | ALAS2 | 1.19 | 0.05 | 0.05 |
| Diff_D7 | BAX | 1.83 | 0.14 | 0.15 | Exp_D11 | BAX | 1.00 | 0.03 | 0.03 |
| Diff_D7 | BAX | 1.54 | 0.10 | 0.10 | Exp_D11 | BAX | 1.01 | 0.01 | 0.01 |
| Diff_D7 | GADD45A | 5.16 | 0.26 | 0.27 | Exp_D11 | GADD45A | 1.00 | 0.10 | 0.11 |
| Diff_D7 | GADD45A | 3.40 | 0.38 | 0.43 | Exp_D11 | GADD45A | 0.89 | 0.17 | 0.21 |
| Diff_D7 | GATA1 | 1.49 | 0.08 | 0.08 | Exp_D11 | GATA1 | 1.00 | 0.08 | 0.08 |
| Diff_D7 | GATA1 | 2.26 | 0.26 | 0.30 | Exp_D11 | GATA1 | 1.09 | 0.14 | 0.16 |
| Diff_D7 | GATA2 | 2.89 | 1.41 | 2.75 | Exp_D11 | GATA2 | 1.00 | 0.41 | 0.70 |
| Diff_D7 | GATA2 | 1.05 | 0.18 | 0.21 | Exp_D11 | GATA2 | NA | NA | NA |
| Diff_D7 | HBB | 29.57 | 1.36 | 1.43 | Exp_D11 | HBB | 1.00 | 0.02 | 0.02 |
| Diff_D7 | HBB | 35.20 | 0.90 | 0.92 | Exp_D11 | HBB | 0.86 | 0.03 | 0.04 |
| Diff_D7 | P21 | 1.38 | 0.08 | 0.08 | Exp_D11 | P21 | 1.00 | 0.09 | 0.10 |
| Diff_D7 | P21 | 1.00 | 0.15 | 0.17 | Exp_D11 | P21 | 0.96 | 0.06 | 0.06 |
| Diff_D7 | P53 | 1.05 | 0.14 | 0.17 | Exp_D11 | P53 | 1.00 | 0.09 | 0.10 |
| Diff_D7 | P53 | 0.86 | 0.03 | 0.03 | Exp_D11 | P53 | 0.94 | 0.06 | 0.07 |

Table 89 confirms that GATA1 and GATA2 are expressed in both progenitor and differentiated cells. Further, pro-apoptotic markers, such as BA and GADD45a, were observed to be expressed at a higher level in differentiated cells compared to CD34+ progenitor cells.

D. Analyzing Knockdown Efficiency

The efficiency of different shRNAs were tested in a conditionally immortalized cord blood erythroblast cell system to mimic DBA. The pTRIPZ was used to transduce these cells with sh1 (dox inducible shRPS19) or sh2 (dox inducible shRPS19), and pLKO was used to transduce a group of cells with sh5 (shRPS19). qRT-PCR was used to determine the level of gene expression in RPS19 mRNA knockdown in each group of cells. The percent of knockdown is shown in Table 90 after 1 week (T1), 2 weeks (T2), and 3 weeks (T3) for each shRNA.

TABLE 90
Percent of RPS19 knockdown in CI cord blood CD34+ cells

| | pLKO sh5 | pTRIPZ sh1 | pTRIPZ sh2 |
|---|---|---|---|
| T1 | 35 | 55 | 25 |
| T2 | 86 | 72 | 54 |
| T3 | 4 | NA | NA |

shRNA against RPS19 possessed a 45-75% knockdown at T1. Cells were observed to experience an increase in the percent of knockdown at T2 in each of the groups. By T3, cells became negatively selected for the knockdown.

These results show that Models 2 and 5 would be the most effective for generating the volume of biomass needed to analyze using next generation sequencing techniques, such as ChIP-seq and RNA-seq. The estimated time to achieve enough biomass for ChIP was about one month for Model 2 and about 2.5 months for Model 5.

### Example 19. Normal CD34+ Progenitor Cells from Peripheral Blood

Human CD34⁺ cells are derived from the peripheral blood and grown in a liquid culture. The length of time for differentiation is increased compared to the length of time for differentiation of the CD34+ cord blood cells. Positive selection of erythroblasts using magnetic beads was used to provide a more homogenous population for genome-wide studies, such as ChIP-seq to identify genomic positions and the composition of genomic signaling centers.

A. Presence of Cell Surface Markers in Expanded Peripheral Blood CD34⁺ Cells

Mapping is initially performed at differentiation stages most affected in the DBA phenotype to build a reference gene expression pattern for human erythropoiesis, such as the BFU/CFU and early erythroblast stages. By day 7 of expansion, BFU/CFU cell markers were observed to be present, and by day 7 of differentiation, early erythroblast cell markers were observed to be present. BFU and CFU markers were present in the expanded CD34⁺ cells described above. BFU cells are known to express CD34 and CD71, and CFU cells are known to express high levels of CD36 and CD71. qRT-PCR was used to measure expression throughout the expansion of the CD34+ peripheral blood cells. Relative quantification of the level of expression of CD71 transferring receptor from day 4 of expansion (exp-D4) to day 1 of differentiation (Diff-D1) is shown in Table 91.

TABLE 91
Relative quantification (RQ) of expression of CD71

| Sample | RQ | Sample | RQ | Sample | RQ | Sample | RQ |
|---|---|---|---|---|---|---|---|
| Exp-D4 | 1.00 | Exp-D6 | 0.80 | Exp-D8 | 0.90 | Exp-D12 | 1.40 |
| Exp-D5 | 1.20 | Exp-D7 | 0.74 | Exp-D11-DMSO-1hr | 1.32 | Diff-D1 | 1.44 |

Relative quantification of the level of expression of CD36 from day 4 of expansion (exp-D4) to day 1 of differentiation (Diff-D1) are shown in Table 92.

TABLE 92
Relative quantification (RQ) of expression of CD36

| Sample | RQ | Sample | RQ | Sample | RQ | Sample | RQ |
|---|---|---|---|---|---|---|---|
| Exp-D4 | 1.00 | Exp-D6 | 0.80 | Exp-D8 | 2.04 | Exp-D12 | 2.90 |
| Exp-D5 | 1.40 | Exp-D7 | 0.74 | Exp-D11-DMSO-1hr | 2.61 | Diff-D1 | 2.41 |

Therefore, the expanded cells expressed markers known to be present in BFU and CFU cells and are the most effected by the DBA phenotype.

### Example 20. Determining the Time Point for Glycophorin a Positive (GYPA+) Selection

qRT-PCR was used to show changes in the levels of expression of GATA1 and GATA2 in both peripheral and cord blood cells to determine a time point for GYPA+ selection. GAPDH was used as an internal control. Table 93 provides the relative quantification of the expression of these genes.

TABLE 93
Relative quantification (RQ) of GATA1 and GATA2

| Cord Blood | | | Peripheral Blood | | |
|---|---|---|---|---|---|
| Sample | Gene | RQ | Sample | Gene | RQ |
| Exp_D 10 | GATA1 | 1.00 | Exp_D 12 | GATA1 | 1.09 |
| | GATA2 | 1.00 | | GATA2 | 0.91 |
| Diff_D 8 | GATA1 | 1.78 | Diff_D 1 | GATA1 | 1.26 |
| | GATA2 | 0.66 | | GATA2 | 0.48 |
| Diff_D 10 | GATA1 | 2.35 | Diff_D 2 | GATA1 | 1.35 |
| | GATA2 | 0.34 | | GATA2 | 0.35 |
| Diff_D 11 | GATA1 | 3.04 | Diff_D 3 | GATA1 | 1.01 |
| | GATA2 | 0.36 | | GATA2 | 0.30 |
| Diff_D 14 | GATA1 | 3.41 | Diff_D 7 | GATA1 | 1.33 |
| | GATA2 | 0.30 | | GATA2 | 0.25 |
| | | | Diff_D 11 | GATA1 | 2.36 |
| | | | | GATA2 | 0.34 |

While GATA1 levels gradually increased during erythroid differentiation in both cord blood and peripheral blood cells, GATA2 levels sharply decreased 60-70% during erythroid differentiation in peripheral cells. Cord blood cells only showed sharp decrease in GATA2 at day 10 of differentiation, while a sharp increase was observed in the peripheral blood cells on day 2 of differentiation.

A. Enriching Erythroid Cells Expressing GATA1

GYPA+ selection was used at day 12 to enrich peripheral erythroid cells expressing GATA1. GYPA+ selection was observed to increase the ratio of GATA1 to GATA2 over 3-fold, i.e. 40× more GATA1 at day 12, as shown in Table 94.

TABLE 94
Relative quantification (RQ) of GATA1
and GATA2 after GYPA+ selection

| | RQ | | | RQ | |
|---|---|---|---|---|---|
| Sample | GATA1 | GATA2 | Sample | GATA1 | GATA2 |
| Exp_D 1 | 1 | 1.08 | Diff_D 1 | 1.26 | 0.48 |
| Exp_D 2 | 1.23 | 1 | Diff_D 2 | 1.35 | 0.35 |
| Exp_D 3 | 1.5 | 0.98 | Diff_D 3 | 1.01 | 0.30 |
| Exp_D 4 | 1.7 | 1.01 | Diff_D 4 | 1.57 | 0.40 |
| Exp_D 5 | 1.24 | 1.18 | Diff_D 7 | 1.33 | 0.25 |
| Exp_D 6 | 1.89 | 1.16 | Diff_D 8 | 1.93 | 0.33 |
| Exp_D 11 | 2.16 | 0.75 | Diff_D 11 | 2.36 | 0.34 |
| Exp_D 12 | 1.09 | 0.91 | Diff_D 12 | 7.29 | 0.21 |

As shown above, GATA2 levels sharply decreases at day 1 (D1) of differentiation. GATA1 levels were observed to gradually increase during erythroid differentiation. Day 7 (D7) of differentiation was chosen as a time point for GYPA+ selection because it provided an early erythroid population, i.e., GATA2 levels were observed to be low. Seven days of differentiation also allowed for a sufficient increase in biomass for ChIP analysis. The erythroid cells were enriched at day 12 (D12) to determine the effectiveness late in differentiation.

### Example 21. Modeling Disease Using Self-Delivering RNA (sdRNA)

Dox removal initiates normal differentiation, and a window for recovery of levels of p53 after efficient knockdown (>95%) with a shP53 should be 3-7 days. As an alternative to using shRNAs, a DBA model was designed using self-delivering RNA (sdRNA) to knockdown p53. sdRNAs provide a transient p53 knockdown, and the level of knockdown can be more easily optimized. sdRNAs targeting additional disease targets, such as RPS19, are also designed and tested.

To validate the efficacy of sdRNA to temporarily alleviate cell death due to RPS19 knockdown, p53 knockdown efficiency and reversibility were assessed.

sdRNAs targeting p53 were introduced to CB-I RES cells. Gene expression of p53 was measured using qRT-PCR at either 48 or 72 hours. Results for the sdRNAs at different concentrations is shown in Table 95.

TABLE 95
Relative quantification (RQ) of p53 during titration of p53 sdRNAs

| Concentration (μM) | 48 hours | 72 hours |
|---|---|---|
| 0.0 (Control) | 1.00 | 1.00 |
| 0.1 | 1.23 | 1.00 |
| 0.5 | 0.81 | 0.84 |
| 1.0 | 0.69 | 0.63 |
| 2.0 | 0.64 | 0.50 |
| 4.0 | 0.61 | 0.43 |

For about 50% knockdown, the sdRNA administered at 2 μM for 72 hours was chosen for further analysis

To determine reversibility of the knockdown, CB-IRES cells were treated with sdRNAs for 48 hours, then washed with PBS p53 levels were monitored 5 days after initial wash. Full recovery knockdown was observed for both 4 μM and 2 μM of sdRNA.

### Example 22. Determining Genomic Position and Composition of Genomic Signaling Centers in Erythroid Cells

Changes in the composition and changes in genomic signaling centers with and without compound treatment were observed in engineered disease models and patient disease cells.

A. ChIP-Seg Targets

Table 96 provides the list of ChIP-seq targets analyzed herein.

TABLE 96
ChIP-seq targets for human erythroblasts

| Chromatin Markers | Transcription factors | | Signaling molecules | |
|---|---|---|---|---|
| H3K4me3 | MYB | MYBL2 | GR (glucocorticoid receptor) | STAT3 - JAK/STAT |
| H3K27Ac | GFIB | NFE2 | AR (androgen receptor) | p53 - p53, mTOR, AMPK |
| H3K4me1 | CEBPG | MLL | ERa (estrogen receptor) | TEAD 1/2 - Hippo |
| H3K27me3 | DDIT3 | FOG1 | LXRa (liver X receptor) | NF-κB (p65) - NF-κB |
| p300 | E2F2 | PTTG1 | FXR (farnesoid X receptor) | CREB1 - MAPK |
| BRD4 | BMI1 | FLI1 | AHR (aryl hydrocarbon receptor) | CREB2 - MAPK |
| | RUNX1 | TAL1 | SXR (steroid and xenobiotic receptor) | c-JUN - TLR, MAPK |
| | MYC | TFDP1 | HIF1a (hypoxia inducible factor) | c-FOS - TLR, MAPK |
| | GATA1 | ELK1 | TCF7L2 (TCF4) - WNT | ELK1 - MAPK |
| | GATA2 | TSC22D3 | CTNNB1 - WNT | ETV4 - ERK MAPK |
| | GFIB | ZNF165 | SMAD2/3 - TGFβ | RBPJK - NOTCH |
| | HES6 | ZNF23 | SMAD4 - TGFβ | SREBP1 - sterol |
| | KLF1 | PU.1 | SMAD1/5/8 - TGFβ | SREBP2 - sterol |
| | LMO2 | | STAT1 - JAK/STAT | |

B. Changes in GSCs Through Cell Differentiation

To produce 3D gene regulatory maps, the following steps are followed: optimization of tissue culture conditions for growth and expansion of CD34⁺ cells, identification of genomic positions and protein composition of GSCs in normal CD34⁺ cells in absence and presence of compounds, determination of genome architecture in these cells, and identification of genomic positions and protein composition of GSCs in a human model of a disease (e.g., erythroblast cells with DBA from shRNA knockdown of RPS19) or primary disease patient samples in the presence and absence of compounds, and demonstration of the capability of compounds to modulate expression of gene targets implicated in the development of a disease by leveraging GSCs. The map of the progenitor culture may also be used to predict whether a response would occur or not when a drug is administered.

To determine the protein composition and genomic position of GSCs in normal primary CD34+ and erythroblasts cells, ChIP-seq and RNA-seq were performed and results are shown for the INs containing GADD45A, RPS19, and CDKN1A. The presence of H3K27Ac, GATA1, GATA2, p53, and ser15 (p53 phosphorylated at serine 15) within these insulated neighborhoods is shown. Cells were collected at day 11 of expansion prior to erythroid differentiation and day 18 after 7 days of differentiation.

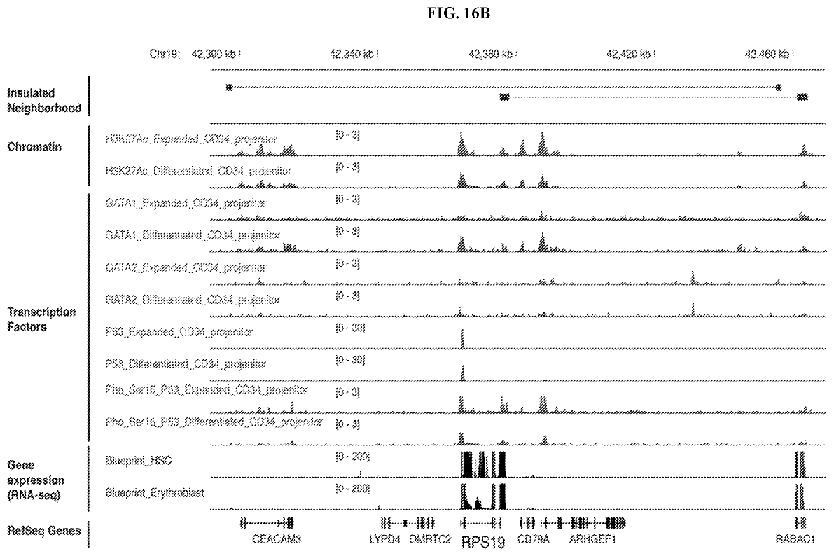

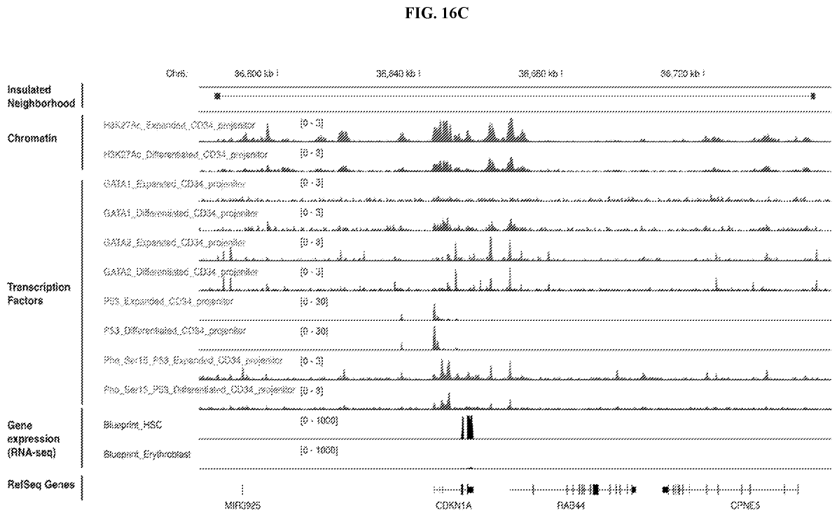

The binding profile in FIG. 16A revealed a significant increase in p53 accumulation at the GADD45A promotor during differentiation. This appears consistent to what has been observed at other apoptotic genes (e.g. BAX). p53 was also abundant at the RPS19 promotor, as shown in FIG. 16B, but levels were similar pre- and post-differentiation. In FIG. 16C, a decrease in the presence of GATA1 was observed, while a decrease in expression of RPS19 was also observed from RNA-seq results.

Therefore, FIG. 16A-16C provided evidence that GATA1/GATA2 is co-expressed at both E11 and D7 and p53 binds the RPS19 and GADD45a promoter. Since these data are from wildtype cells, results indicate a mechanism by which apoptotic genes are ‘poised’ to be triggered in this lineage. Since present results do not show a clear timepoint for the switch from GATA2 to GATA1, further differentiation may be needed.

C. Binding Profiles

Collating the results of FIG. 16A-16C showed that p53 was bound to more peaks in erythroid cells compared to hematopoietic cells (1485 vs. 743). A majority of p53 was also observed to bind non-promoter regions in hematopoietic stem cells. In fact, 3.6% of the p53 peaks are in exon regions, 23.6% are in promoter regions, 29.9% are in intron regions, and 42.9% are in intergenic regions.

For all the p53 binding sites, there is significant overlap between p53 binding in hematopoietic stem cells and erythroblasts. Five hundred seventy-eight of the p53 peaks were shared in both hematopoietic stem cells and erythroblasts. 3.6% of the p53 peaks are in exon regions, 20.8% are in promoter regions, 31.5% are in intron regions, and 44.1% are in intergenic regions. One hundred one of the 385 binding sites found in promoter regions were shared by hematopoietic stem cells and erythroblasts.

D. Gene Ontology (GO) Analysis of the p53 Binding Sites

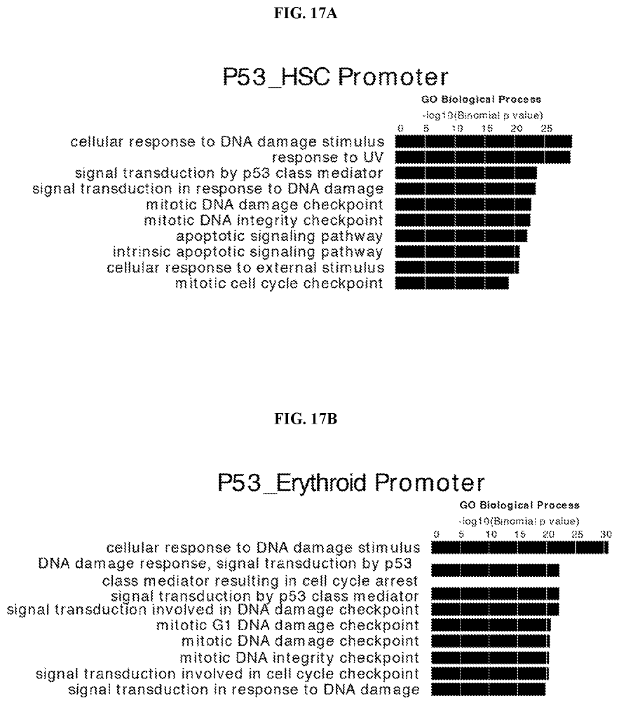

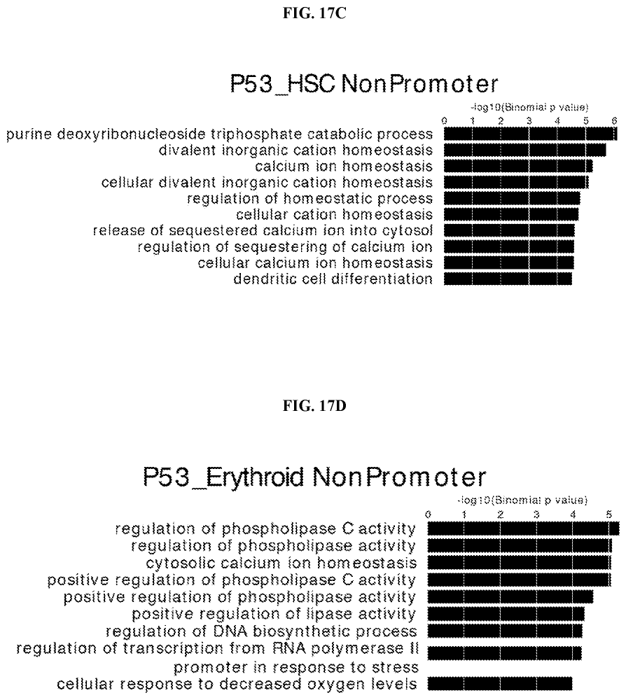

FIG. 17A-17D show the log 10 p-values for genomic annotation of the binding sites of p53 (log 10 p-value≤0.05 were considered significant). FIG. 17A shows that most of the binding sites in a hematopoietic stem cell promoter region are likely associated with cellular response to DNA damage stimulus and response to UV exposure. Alternatively, these binding sites may be associated with signal transduction by p53 class mediator, signal transduction in response to DNA damage, mitotic DNA damage checkpoint, mitotic DNA integrity checkpoint, apoptotic signaling pathway, intrinsic apoptotic signaling pathway, cellular response to external stimuli, and mitotic cell cycle checkpoint in descending frequency.

FIG. 17B shows that the p53 binding sites of erythroid cells are most likely located in promoter regions associated with cellular response to DNA damage stimulus. Alternatively, the binding sites are associated with cellular response to DNA damage stimulus, DNA damage response and signal transduction by p53, class mediator resulting in cell cycle arrest, signal transduction by p53 class mediator, signal transduction involved in DNA damage checkpoint, mitotic G1 DNA damage checkpoint, mitotic DNA damage checkpoint, mitotic DNA integrity checkpoint, signal transduction involved in cell cycle checkpoint, and signal transduction in response to DNA damage in descending frequency.

In contrast to p53 binding sites in promoter regions of both hematopoietic stem cells and erythroid cells, FIG. 17C shows that p53 binding sites in non-promoter regions of hematopoietic stem cells were observed in regions of the genome most likely associated with the purine deoxyribonucleoside triphosphate catabolic process. Alternatively, p53 binding sites were also in non-promoter regions of hematopoietic stem cells most likely associated with divalent inorganic cation homeostasis, calcium ion homeostasis, cellular divalent inorganic cation homeostasis, regulation of homeostatic process, cellular cation homeostasis, release of sequestered calcium ion into cytosol, regulation of sequestering calcium ion, cellular calcium ion homeostasis, and dendritic cell differentiation in descending frequency.

Further, FIG. 17D shows that p53 binding sites in non-promoter regions of erythroid cells were most frequent in regions most likely associated with regulation of phopholipase C activity. P53 binding sites in non-promoter regions of erythroid cells were also potentially associated with regulation of phospholipase activity, cytosolic calcium ion homeostasis, positive regulation of phospholipase C activity, positive regulation of phospholipase activity, positive regulation of lipase activity, regulation of DNA biosynthesis process, regulation of transcription from RNA polymerase II, promoter in response to stress, and cellular response to decrease oxygen levels in descending frequency.

Therefore, p53 was observed to bind the promoter regions of apoptotic genes in both hematopoietic stem cells and differentiated erythroid cells, and calcium ion homeostasis-related genes are enriched in non-promoter p53 peaks.

E. Overlap with Active Enhancers

Each identified p53 non-promoter binding site was extended 500 base pairs upstream and downstream, and the percentage of overlap of these regions with H3K27Ac, a histone modification associated with active enhancers, was determined. Of the 566 p53 binding sites observed in non-promoter regions of hematopoietic stem cells, 47.5% of them overlapped with H3K27Ac. Of the 1170 p53 binding sites observed in non-promoter regions of erythroid cells, 37.7% overlap with H3K27Ac. Further no enriched biological processes were observed through quantification of genomic annotation of the p53 binding sites overlapping with H3K27Ac.

### Example 23. Compound Treatment of Conditionally Immortalized and Normal CD34⁺ Cells

The conditionally immortalized CD34+ cord blood primary cells (CB-CI-IRES-CD34+) and TF1 cells were treated with compounds to rescue the phenotype of DBA in the disease model. Doses of the compounds were titrated to determine the concentration needed to stimulate a response in the cells.

A. Cell Toxicity

A cell viability ATP assay was performed with different concentrations of Nutlin 3, Nutlin 3a, pifithrin alpha (pifithrin-a), and cyclic pifithrin alpha to determine the IC50. Cells treated with DMSO served as a control. Amount of ATP for concentrations of 100, 50, 25, 12.5, 6.252, 3.125, 1.5, 0.7, 0.35, 0.175, and 0.08625 μM are shown in Table 97. Measurements were performed in triplicate.

TABLE 97
ATP cell viability assay for CD34+ cells

| | | | | | | | | | | | | |
|---|---|---|---|---|---|---|---|---|---|---|---|---|
| μM | 100 | 50 | 25 | 12.5 | 6.252 | 3.125 | 1.5 | 0.7 | 0.35 | 0.175 | 0.08625 | DMSO |

Nutlin 3

| | | | | | | | | | | | | |
|---|---|---|---|---|---|---|---|---|---|---|---|---|
| 1 | 292464 | 448385 | 488775 | 538130 | 554494 | 608248 | 612632 | 624991 | 458003 | 612502 | 330857 | 562181 |
| 2 | 300163 | 494399 | 563081 | 610101 | 645140 | 673589 | 669622 | 692198 | 738460 | 725724 | 645570 | 628762 |
| 3 | 301403 | 461970 | 517641 | 574004 | 567858 | 595367 | 618466 | 601983 | 618870 | 612658 | 672858 | 616130 |
| Avg. | 298010 | 468251 | 523166 | 574078 | 589164 | 625735 | 633573 | 639724 | 605111 | 650295 | 549762 | 602358 |

Pifithrin alpha

| | | | | | | | | | | | | |
|---|---|---|---|---|---|---|---|---|---|---|---|---|
| 1 | 523409 | 609905 | 632781 | 674137 | 680518 | 664676 | 640507 | 652343 | 661374 | 697888 | 672962 | 563212 |
| 2 | 528238 | 621558 | 628005 | 648468 | 671214 | 649825 | 648689 | 645401 | 678900 | 672793 | 670418 | 583909 |
| 3 | 518281 | 588738 | 624495 | 626948 | 622759 | 624860 | 622315 | 392544 | 618740 | 642882 | 584757 | 583622 |
| Avg. | 523309 | 606734 | 628427 | 649851 | 658164 | 646454 | 637170 | 563429 | 653005 | 671188 | 642712 | 576914 |

Cyclic pifithrin alpha

| | | | | | | | | | | | | |
|---|---|---|---|---|---|---|---|---|---|---|---|---|
| 1 | 555382 | 627013 | 657355 | 671488 | 681093 | 679344 | 658333 | 624442 | 631985 | 630354 | 633812 | 543911 |
| 2 | 642595 | 693777 | 728764 | 722696 | 740862 | 732131 | 695395 | 666894 | 695056 | 632494 | 560445 | 617187 |
| 3 | 725828 | 747791 | 779711 | 771607 | 742101 | 764339 | 737638 | 682685 | 671814 | 636279 | 675142 | 631594 |
| Avg. | 641268 | 689527 | 721943 | 721930 | 721352 | 725271 | 697122 | 658007 | 666285 | 633042 | 623133 | 597564 |

Nutlin 3a

| | | | | | | | | | | | | |
|---|---|---|---|---|---|---|---|---|---|---|---|---|
| 1 | 429802 | 463223 | 465507 | 496305 | 481101 | 539839 | 559728 | 603745 | 580086 | 617918 | 588229 | 616639 |
| 2 | 424673 | 463614 | 460965 | 442760 | 491124 | 515592 | 525315 | 579055 | 562298 | 572490 | 590199 | 571564 |
| 3 | 359384 | 421058 | 448776 | 432516 | 471758 | 513726 | 503547 | 550788 | 569228 | 567231 | 569267 | 576967 |
| Avg. | 404620 | 449298 | 458416 | 457194 | 481328 | 523052 | 529530 | 577863 | 570537 | 585880 | 582565 | 588390 |

A cell viability assay was also carried out for TF1 cells to determine the optimal concentration prior to cell toxicity for each compound. Results are shown in Table 98.

TABLE 98
ATP cell viability assay for TF1 cells

| | | | | | | | | | | | |
|---|---|---|---|---|---|---|---|---|---|---|---|
| μM | 100 | 50 | 25 | 12.5 | 6.252 | 3.125 | 1.5 | 0.7 | 0.35 | 0.175 | 0.08625 |

Pifithrin alpha

| | | | | | | | | | | | |
|---|---|---|---|---|---|---|---|---|---|---|---|
| 1 | 782582 | 812180 | 1041547 | 1244226 | 1261048 | 1337181 | 1366687 | 1144824 | 1128629 | 1327798 | 1384592 |
| 2 | 976310 | 788129 | 1058499 | 1209017 | 1251586 | 1328516 | 1336477 | 1238562 | 1381564 | 1366309 | 1395162 |
| 3 | 916449 | 831664 | 996093 | 1125928 | 1214015 | 1225669 | 1270378 | 1250647 | 1312856 | 1323453 | 1250816 |
| Avg. | 891780 | 810658 | 1032046 | 1193057 | 1242216 | 1297122 | 1324514 | 1211344 | 1274350 | 1339187 | 1343523 |

Cyclic Pifithrin alpha

| | | | | | | | | | | | |
|---|---|---|---|---|---|---|---|---|---|---|---|
| 1 | 1292289 | 1399978 | 1333723 | 1374544 | 1386589 | 1380129 | 1323936 | 1380181 | 1379437 | 1328647 | 1266411 |
| 2 | 1394301 | 1395554 | 1425739 | 1457698 | 1399051 | 1358414 | 1425034 | 1423050 | 1425399 | 1457633 | 1417713 |
| 3 | 1313065 | 1345794 | 1382178 | 1366440 | 1353389 | 1354916 | 1412128 | 1401192 | 1367940 | 1400082 | 1369050 |
| Avg. | 1333218 | 1380442 | 1380547 | 1399561 | 1379676 | 1364486 | 1387033 | 1401474 | 1390925 | 1395454 | 1351058 |

Nutlin 3a

| | | | | | | | | | | | |
|---|---|---|---|---|---|---|---|---|---|---|---|
| 1 | 23934 | 1246588 | 1518028 | 1340587 | 1460412 | 1326585 | 1501481 | 1465593 | 1501089 | 1387750 | 1477730 |
| 2 | 23542 | 1204019 | 1486252 | 1403123 | 1231790 | 1450508 | 1426000 | 1321104 | 1461456 | 1481279 | 1426195 |
| 3 | 27797 | 1331152 | 1420453 | 1389877 | 1364965 | 1385714 | 1398203 | 1325541 | 1375796 | 1406790 | 1238001 |
| Avg. | 25091 | 1260586 | 1474911 | 1377862 | 1352389 | 1387602 | 1441895 | 1370746 | 1446114 | 1425273 | 1380642 |

Nutlin 3

| | | | | | | | | | | | |
|---|---|---|---|---|---|---|---|---|---|---|---|
| | 27666 | 879100 | 1441451 | 1455179 | 1468908 | 1409570 | 1412845 | 1421014 | 1391417 | 1405054 | 1354903 |
| | 29245 | 933388 | 1512469 | 1541675 | 1560088 | 1515470 | 1497031 | 1519190 | 1520129 | 1512482 | 1472614 |
| | 23602 | 703030 | 1435866 | 1457750 | 1481632 | 1443604 | 1447193 | 1423442 | 1329299 | 1422972 | 1411853 |
| Avg. | 26837 | 838506 | 1463262 | 1484868 | 1503543 | 1456215 | 1452356 | 1454549 | 1413615 | 1446836 | 1413123 |

B. Dose Titration

Dose titration was performed as described in Example 1. Three doses of each compound that did not result in cell toxicity in the results above were chosen to determine the amount of the compound needed to elicit a response in RPS19 knockdown cells that resulted in a rescued DBA phenotype by reducing elevated p53 levels caused by the DBA phenotype. Phenotype rescue was determined by measuring p53 levels by qRT-PCR analysis. It was expected that the low dose (3.125 μM) would result in no change in p53, that the medium dose (6.25 μM) would result in some change in p53, and the maximal dose would result in a decrease in p53. Wildtype TF1 or conditionally immortalized erythroblast cord blood cells were transduced with either pSMART PGK GFP shNT (dox inducible), pSMART PGK GFP shRPS19_1 (dox inducible), pSMART PGK GFP shRPS19_2 (dox inducible), pSMART EF1alpha GFP shNT (dox inducible), pLKO RPS19_sh4 (constitutive), pLKO RPS19_sh5 (constitutive), pLKO shNT (constitutive), or pLKO RPS19_sh1 (constitutive). An aliquot of knockdown cells treated only with DMSO was used as a control. The 8 samples of knockdown cells are also compared to a wildtype TF1 cell control. Knockdown resulted in the cell densities shown in Table 99.

TABLE 99
Cell densities

| | Cell density | | Cell density |
|---|---|---|---|
| Plasmid | [cells/ml] | Plasmid | [cells/mll |
| pSMART PGK GFP | 517,000 | pLKO RPS19_sh4 | 124,000 |
| shNT | | | |
| pSMART PGK GFP | 274,000 | pLKO RPS19_sh5 | 962,000 |
| shRPS19_1 | | | |
| pSMART PGK GFP | 1,100,000 | pLKO shNT | 1,190,000 |
| shRPS19_2 | | | |
| pSMART EF1alpha | 1,410,000 | pLKO RPS19_sh1 | 28,000 (paren- |
| GFP shNT | | | tal flask) |

C. Changes in Biological Activity

Compound dilutions were performed as described in Example 1. Compound Nutlin 3, Pifithrin alpha, Nutlin 3a, and Cyclic-pifithrin alpha were administered in a 10 μM dose. In a 6-well format, there were 1 million cells/well=1 million cells/ml=1000 cells/pl. Eight ml of RPMI+GMCSF was added to each well. Table 100 shows changes in cell viability.

TABLE 100
Fold changes in cell viability

| | Nutlin | Pifithrin | Nutlin | Cyclic pifithrin | |
|---|---|---|---|---|---|
| | 3 | alpha | 3a | alpha | DMSO |
| TF1 | 1.07 | 1.23 | 1.08 | 1.12 | 1.00 |
| CD34+ | 0.78 | 1.08 | 0.81 | 1.17 | 1.00 |

TF1 cells did not significantly respond to p53 pathway activators Nutlin 3/Nutlin 3a or inhibitors Pifithrin alpha/cyclic. CD34+ cells treated with Nutlin 3 and Nutlin 3a had reduced biological activity, while Pifithrin had little or no effect.

### Example 24. Bioinformatics Platform

The following Example provides the process used in creating a bioinformatics platform for automating drug selection and development based on canonical pathways.

A. Compound Database

At least 150 compounds that manipulate signaling pathways were initially chosen to be included in the database. 8226 drugs, 2262 target proteins, 291 KEGG pathways, and 46 signaling pathways were considered for inclusion. Relevant considerations for selection of compounds includes: coverage of the signaling biology space, likelihood of providing information and new insights, cost efficiency, chance of finding a new molecule, chance of finding repurposing candidate compounds, chance of finding clinically relevant molecules, and coverage of the drug/chemical space.

Target proteins are those whose mRNA levels were expressed greater than 1 fragment per kilobase of transcript per million (FPKM) mapped reads. Target proteins expressed in the liver (mRNA FPKM>1) were associated with 3191 drugs, 1379 target proteins, 289 KEGG pathways, and 46 signaling pathways.

Fifty signaling pathways and nuclear receptor subfamilies were selected. An agonist and antagonist were identified for each pathway. The list of 50 pathways was compared with the ChIP-seq targets in Tables 41 and 96 to confirm that ChIP-seq information would be gathered for each pathway. Table 101 shows the number of agonists and antagonists identified for each canonical pathway.

TABLE 101
Number of agonists and antagonists for canonical KEGG pathways

| Signaling pathways | Agonists | Antagonists |
|---|---|---|
| Adipocytokine signaling pathway | 20 | 20 |
| Adrenergic signaling in cardiomyocytes | 58 | 155 |
| AGE-RAGE signaling pathway in diabetic | 2 | 45 |
| complications | | |
| AMPK signaling pathway | 56 | 71 |
| B cell receptor signaling pathway | 2 | 25 |
| Bile secretion | 5 | 46 |
| Calcium signaling pathway | 117 | 344 |
| cAMP signaling pathway | 118 | 270 |
| cGMP-PKG signaling pathway | 112 | 177 |
| Chemokine signaling pathway | 1 | 34 |
| Epithelial cell signaling pathway in | 0 | 31 |
| Helicobacter pylori infection | | |
| ErbB signaling pathway | 1 | 31 |
| Estrogen signaling pathway | 65 | 50 |
| Fc epsilon RI signaling pathway | 2 | 25 |
| FoxO signaling pathway | 11 | 35 |
| Glucagon signaling pathway | 12 | 16 |
| GnRH signaling pathway | 7 | 52 |
| Hedgehog signaling pathway | 2 | 4 |
| HIF-1 signaling pathway | 10 | 40 |
| Hippo signaling pathway | 0 | 9 |
| Insulin signaling pathway | 10 | 34 |
| Jak-STAT signaling pathway | 21 | 19 |
| MAPK signaling pathway | 4 | 81 |
| mTOR signaling pathway | 4 | 23 |
| Neuroactive ligand-receptor interaction | 300 | 380 |
| Neurotrophin signaling pathway | 4 | 36 |
| NF-kappa B signaling pathway | 1 | 73 |
| NOD-like receptor signaling pathway | 0 | 20 |
| Notch signaling pathway | 3 | 3 |
| Steroid and xenobiotic receptor pathway | 6 | 0 |
| (NR1I2) | | |
| Oxytocin signaling pathway | 14 | 111 |
| p53 signaling pathway | 0 | 12 |
| Pathways in cancer | 61 | 149 |
| Phosphatidylinositol signaling system | 1 | 13 |
| Phospholipase D signaling pathway | 17 | 50 |
| PI3K-Akt signaling pathway | 41 | 145 |
| PPAR signaling pathway | 29 | 7 |
| Prolactin signaling pathway | 26 | 26 |
| Rap1 signaling pathway | 33 | 123 |
| Ras signaling pathway | 13 | 100 |
| Retrograde endocannabinoid signaling | 21 | 109 |
| RIG-I-like receptor signaling pathway | 0 | 14 |
| Sphingolipid signaling pathway | 30 | 41 |
| T cell receptor signaling pathway | 2 | 30 |
| TGF-beta signaling pathway | 0 | 11 |
| Thyroid hormone signaling pathway | 36 | 36 |
| TNF signaling pathway | 2 | 71 |
| Toll-like receptpr signaling pathway | 8 | 26 |
| VEGF signaling pathway | 1 | 75 |
| Wnt signaling pathway | 3 | 8 |

Table 102 provides examples of pathways with an associated agonist and antagonist compounds.

TABLE 102
Examples of agonists and antagonists of signaling pathways

| Signaling Pathways | Compound | Type | Gene | Compound | Type | Gene |
|---|---|---|---|---|---|---|
| AMPK signaling | Aleglitazar | Agonist | PPARG | Simvastatin | Inhibitor | HMGCR |
| Neuroactive ligand-receptor interaction | Prednisone | Agonist | NR3C1 | Budesonide | Antagonist | NR3C1 |
| Wnt signaling | Bezafibrate | Agonist | PPARD | Bosutinib | Inhibitor | CAMK2G |
| NF-kappa B signaling | Rasagiline | Activator | BCLXL | Venetoclax | Inhibitor | BCLXL |

Compounds were also selected from those that have faded Phase II or Phase III trials for efficacy including the 45 compounds in Table 35 that faded a Phase III trial.

B. Genetic Disease Database

About 1100 diseases with causal genetic mutation information are identified, including diseased tissue, prevalence, inheritance pattern, genetic mutation, pathogenesis, diagnosis and therapy. One source of the genetic disease database was the Genetics Home Reference from the U.S. National Library of Medicine (https://ghr.nlm.nih.gov).

Approachable diseases in this platform including coding region mutated genes and GSC hyper-activation or hypo-activation diseases. Examples of approachable liver diseases include Factor V deficiency Leiden (thrombophilia), which is an autosomal dominant disease resulting from an F5 gain of function mutation. Familial hypercholesterolemia is also an autosomal dominant disease resulting from an LDLR haploinsufficiency in 95% cases, a PCSK9 gain of function mutation, and/or an APOB haploinsufficiency. Additional examples of approachable autosomal dominant diseases include acute intermittent porphyria resulting from HMBS haploinsufficiency, and fibronectin glomerulopathy resulting from an FN1 haploinsufficiency.

For coding region mutated genes, methods for elevating expression levels of these genes may be determined. Table 103 provides examples of approachable conditions that are the result of a type of disease inheritance pattern.

TABLE 103
Approachable disease characteristics

| Inheritance pattern | Occurrences | Approachable condition |
|---|---|---|
| Autosomal recessive | 441 | Partially functional protein |
| disease | | remained |
| X-linked recessive | 44 | Partially functional protein |
| disease in male | | remained |
| Autosomal dominant | 346 | Haploinsufficiency, the mutant |
| disease | | allele is not toxic |
| X-linked dominant | 28 | Partially functional protein |
| disease in male | | remained |
| X-linked dominant | | Haploinsufficiency, the mutant |
| disease in female | | allele is not toxic |

For GSC hyper-activation or hypo-activation diseases, diseases having the similar pathogenesis are identified. Therefore, a single pathway may be identified that affects multiple diseases.

C. Determining Predictability of Relationships

ChIP-seq targets that were validated in HepG2 cells as described above were compared to published data for quality control. ChIP-seq targets validated herein included: histone modification H3K27Ac and transcription factors CREB1, CTCF, ERa, HIF1A, HNF1, HNF3b, HNF4A, HNF6, NFKB, P53, SERBP1, SMAD23, STAT1, TCF4, and TEAD1.

Histone modifications H3K27ac, H3K27me3, H3K36me3, H3K4me1, H3K4me2, H3K4me3, H3K9me3, H3K79me2, and H3K9ac and transcription factors ATF3, HDAC2, SIN3A, BHLHE40, HNF4A, SP1, CEBPB, HNF4G, SREBF1, CHD2, HSF1, SRF, JUN, JUND, TAF1, MYC, MAFF, TBP, CTCF, MAFK, TCF12, ELF1, NRF1, TCF7L2, ESRRA, REST, NR2C2, FOSL2, EP300, USF1, FOXA1, PPARGC1A, USF2, FOXA2, POLR2A, ZBTB33, GABPA, RAD21, ZNF274, NR3C1, RFX5, H2AZ, and RXRA were identified from ENCODE (Encyclopedia of DNA Elements) Consortium (www.encodeproject.org) and DEEP Consortium (www.deutsches-epigenom-programm.de) as potential ChIP-seq targets.

These lists were observed to share three targets: CTCF, H3K27Ac, and HNF4A. A scatterplot of ChIP-seq signal per 10 kb bin (log 2) is used to determine a correlation value between ChIP-seq results for each of these targets and ChIP-seq results from published data. The correlation among ChIP-seq results from different published sources is also determined.

D. Application of Machine Learning

Machine learning is used to provide in silico prioritization of genome interactions, estimates of confidence for an interaction, and an iterative/systemic framework for identifying previously unidentified interactions.

### Example 25. Building Gene Signaling Networks (GSN)

A study (n=2) of gene expression changes in 56 and 60 genes in the canonical insulin signaling, nuclear receptor signaling and WNT signaling pathways, was conducted to evaluate the correlation of actual effect on expression (up or down) to the known canonical pathway association of each of the 56 or 60 genes. If expression went up or down to a significant degree, then the score was counted as positive. The results are shown in Table 104.

TABLE 104
OE Scores

| | OE SCORE (Occupancy-Expression Score) |
|---|---|
| Insulin | 26/56 (46%); 21/60 (35%) |
| Nuclear | 35/56 (63%); 26/60 (43%) |
| WNT | 24/56 (43%); 31/60 (52%) |

The data revealed that alteration in expression and concomitant assignment to a specific known canonical signaling pathway is not completely reliable. Most interesting were the findings associated with the nuclear receptor signaling pathway. Proteins in this pathway are, by definition, localized to a specific cellular location, i.e., the nucleus, and therefore found to be more highly correlated with a positive association with a change in expression. The data suggest that canonical pathway designation may not be the best predictor or basis of hypothesis testing for mapping gene signaling.

### EQUIVALENTS AND SCOPE

Those skilled in the art will recognize or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

LENGTHY TABLES
The patent application contains a lengthy table section. A copy of the table is available in electronic form from the USPTO web site (https://seqdata.uspto.gov/?pageRequest=docDetail&DocID=US20210254056A1). An electronic copy of the table will also be available from the USPTO upon request and payment of the fee set forth in 37 CFR 1.19(b)(3).

1. A method of altering signaling of a primary neighborhood gene encoded within an insulated neighborhood, wherein the primary neighborhood gene is selected from those in any of Tables 1-9, said method comprising one or more of the following:
a. disrupting a primary upstream or primary downstream boundary of the insulated neighborhood;
b. altering one or more regulatory sequence regions (RSRs) or portions thereof of the encoded primary neighborhood gene;
c. duplicating one or more RSRs or portions thereof of the encoded primary neighborhood gene;
d. inhibiting or reducing the expression and/or activity of one or more signaling molecules associated with the RSR of the primary neighborhood gene;
e. activating or increasing the expression and/or activity of one or more signaling molecules associated with the RSR of the primary neighborhood gene; and/or
f. altering one or more of the upstream or downstream neighborhood genes or its RSR of the insulated neighborhood.

2. The method of claim 1, wherein the insulated neighborhood is a minimal insulated neighborhood.

3. The method of claim 1, further comprising contacting a genomic system that includes the insulated neighborhood with a stimulus.

4. The method of claim 3, wherein the stimulus is at least one selected from group consisting of: a small molecule, a biologic, an antibody, and an environmental condition.

5. The method of claim 4, wherein the stimulus is selected from those described herein.

6. An isolated cell having at least one insulated neighborhood altered in any manner of any of (a)-(f) of claim 1.

7. A method of altering the penetrance of a gene comprising altering the structure of one or more insulated neighborhoods that encompass the gene.

8. A method of predicting one or more treatment liabilities of a therapeutic agent comprising determining the signaling signature of one or more primary neighborhood genes which are differentially expressed upon treatment with the therapeutic agent compared to an untreated control.

9. The method of claim 8, further comprising altering the signaling signature of said one or more primary neighborhood genes which are differentially expressed upon treatment with the therapeutic agent compared to an untreated control.

10. The method of claim 9, wherein the signaling signature is altered by a method selected from the group consisting of increasing the level of a signaling molecule, decreasing the level of a signaling molecule, editing one or more RSRs, altering an IN boundary, affecting a downstream target, and mutating a genomic signaling center.

11. The method of claim 10, wherein signaling molecules to be increased or decreased comprise one or more transcription factors selected from those listed in Table 22.

12. A method of reducing or eliminating one or more treatment liabilities of a therapeutic agent comprising altering the penetrance of a primary neighborhood gene or its RSRs.

13. The method of any one of claim 8 or claim 12, wherein the treatment liability is selected from toxicity, short half-life, and lack of efficacy.

14. A method of altering expression of gene selected from any of those listed in Tables 1-9 in a liver cell comprising contacting said liver cell with a compound selected from the group consisting of any of those taught herein.

15. A method of stratifying or selecting patients for treatment with a compound selected from the group consisting of any of the compounds described herein.

16. A method of screening cell for response to a stimulus by measuring differential gene expression between a group of the cells contacted with the stimulus and a group of the cells not contacted with the stimulus, wherein the stimulus comprises any of those selected from the group consisting of any compound described herein.

17. A method of altering the gene expression attendant to an insulated neighborhood comprising altering a genomic signaling center, the method comprising using a CRISPR/Cas9 system to change the genomic signaling center binding site.

18. The method of claim 17, wherein the change restores a mutation in the genomic signaling center binding site.

19. A method of creating a genomic signaling centers in a genome, the method comprising:
altering a CTCF site to disrupt an enhancer-promoter interaction of a first insulated neighborhood, wherein the enhancer is available for interaction with a different promoter to form a genomic signaling center.

20. The method of claim 19, wherein the CTCF site is altered using a CRISPR/Cas9 enzyme.

21. A method of modulating gene expression in a cell, the method comprising:
contacting the cell with a perturbation stimulus targeting at least one occupancy-dependent signaling center, wherein the occupancy-dependent signaling center comprises a region of the genome bound by at least 2 signaling proteins and comprises:
i) a H3K27 chemical modification, or
ii) independently at least one of a bromodomain-containing protein (Brd), a transcriptional coactivator, and at least two master transcription factors bound to the region, thereby modulating gene expression.

22. The method of claim 21, wherein the bromodomain-containing protein (Brd) is selected from the group consisting of Brd2, Brd3, and Brd4.

23. The method of claim 21, wherein the H3K27 chemical modification is acetylation or methylation.

24. The method of claim 21, wherein the transcriptional coactivator is p300.

25. The method of claim 21, wherein the occupancy-dependent signaling center comprises a nucleic acid sequence selected from SEQ ID NOs: 32,627-71,281.

26. The method of claim 21, wherein the perturbation stimulus is at least one selected from the group consisting of a CRISPR/Cas system, a zinc finger nuclease, a transcription activator-like effector nuclease (TALEN), and a hybridizing oligonucleotide.

27. The method of claim 21, wherein the perturbation stimulus binds the occupancy-dependent signaling center.

28. The method of claim 27, wherein the perturbation stimulus is antisense to the nucleic acid sequence of the occupancy-dependent signaling center.

29. The method of claim 21, further comprising mutating the nucleic acid sequence of the occupancy-dependent signaling center.

30. The method of claim 21, wherein the contacting afters the occupancy of the occupancy-dependent signaling center.

31. The method of claim 30, wherein the contacting alters genome architecture in the cell.

32. The method of claim 31, wherein the contacting alters gene looping.

33. The method of claim 21, wherein the perturbation stimulus is at least one stimulus described herein.

34. A method of altering the occupancy of a signaling center, the method comprising:
(a) contacting the cell with a perturbation stimulus targeting at least one occupancy-dependent signaling center comprising a nucleic acid sequence selected from SEQ ID NOs: 32,627-71,281.

35. The method of claim 34, wherein the perturbation stimulus is at least one selected from the group consisting of a CRISPR/Cas system, a zinc finger nuclease, a transcription activator-like effector nuclease (TALEN), and a hybridizing oligonucleotide.

36. The method of claim 34 or claim 35, wherein the perturbation stimulus binds at least a portion of the occupancy-dependent signaling center.

37. The method of claim 36, wherein the perturbation stimulus is antisense to the portion of the occupancy-dependent signaling center.

38. A composition comprising an oligonucleotide that binds to at least a portion of an occupancy-dependent signaling center comprising a nucleic acid sequence selected from SEQ ID NOs: 32,627-71,281.

39. The composition of claim 38, wherein the oligonucleotide is antisense to the nucleic acid sequence of the occupancy-dependent signaling center.

40. A pharmaceutical composition comprising the composition of claim 38 and a pharmaceutically acceptable excipient.

41. A method of treating a disease in a subject, the method comprising:
(a) administering to the subject the pharmaceutical composition of claim 40, wherein the occupancy-dependent signaling center controls expression of at least one gene associated with the disease.

42. The method of claim 41, wherein the gene is a protein-coding gene.

43. The method of claim 21, wherein the gene is a non-protein-coding gene.

44. A method of perturbing a signaling pathway of a cell, the method comprising:
(a) contacting the cell with a perturbation stimulus that alters the occupancy of an occupancy-dependent signaling center comprising a nucleic acid sequence selected from SEQ ID NOs: 32,627-71,281.
